(19) United States
(12) Patent Application Publication
(10) Pub. No.: US 2007/0020637 A1
(43) Pub. Date: Jan. 25, 2007

(54) Full-length cDNA

(75) Inventors: Takao Isogai, Ibaraki (JP); Tomoyasu Sugiyama, Tokyo (JP); Tetsuji Otsuki, Tokyo (JP); Al Wakamatsu, Chiba (JP); Hiroyuki Sato, Osaka (JP); Shizuko Ishii, Hokkaido (JP); Junichi Yamamoto, Chiba (JP); Yuko Isono, Chiba (JP); Keiichi Nagai, Tokyo (JP); Ryotaro Irie, Saitama (JP)

Correspondence Address: FOLEY AND LARDNER;SUITE 500, 3000 K STREET NW, WASHINGTON, DC, 20007, US

(73) Assignee: RESEARCH ASSOCIATION FOR BIOTECHNOLOGY

(21) Appl. No.: 10/760,320

(22) Filed: Jan. 21, 2004

Related U.S. Application Data
(60) Provisional application No. 60/476,227, filed on Jun. 6, 2003.
(60) Provisional application No. 60/447,287, filed on Feb. 14, 2003.

(30) Foreign Application Priority Data
Jan. 21, 2003 (JP) 2003-102206
May 9, 2003 (JP) 2003-131392

(51) Int. Cl. C12Q 1/68; C07H 21/04

(52) U.S. Cl. 435/6; 536/23.1

(57) ABSTRACT

Novel full-length cDNAs are provided. 2,495 cDNA derived from human have been isolated. The full-length nucleotide sequences of the cDNA and amino acid sequences encoded by the nucleotide sequences have been determined. Because the cDNA of the present invention are full-length and contain the translation start site, they provide information useful for analyzing the functions of the polypeptide.

### FIELD OF THE INVENTION

The present invention relates to polynucleotides encoding novel polypeptides, the polypeptides encoded by these polynucleotides, and new uses of these.

### BACKGROUND OF THE INVENTION

The genomic DNAs of various living organisms are currently being sequenced and analyzed all over the world. The entire genomic sequences of more than 40 species of prokaryotes, a lower eukaryote, yeast, a multicellular eukaryote, C. elegans, and a higher plant, arabidopsis, and such have already been determined. Analysis of the human genome, presumed to have three billion base pairs, was advanced under global cooperative organization, and a draft sequence was disclosed in 2001. In 2003 the complete structure had been elucidated and publically disclosed. A genome is a blueprint for highly complicated living organisms. The aim in determining a genomic sequence is to reveal the function and regulation of all genes, and to understand living organisms as a network of interactions between genes, proteins, cells or individuals. Understanding living organisms through the genomic information of various species is not only academically important, but also socially significant from the viewpoint of industrial application.

However, simply determining a genomic sequence will not reveal the function of all genes. For example, in the case of yeast, the function of only approximately half of the 6000 genes predicted on the basis of genomic sequence have been deduced. The human genome has been estimated to contain about 30,000 to 40,000 genes. Further, 100,000 or more types of mRNAs are said to exist when variants produced by alternative splicing are taken into consideration. Therefore, it is desirable to establish “a high throughput system for analysis of gene functions” which allows rapid and efficient identification of the functions of vast amounts of genes obtained by genomic sequencing.

Many genes in the eukaryotic genome are split by introns into multiple exons. Thus, it is difficult to correctly predict the structure of an encoded protein based solely on genomic information. In contrast, cDNA, which is produced from mRNA that lacks introns, encodes a protein as a single continuous amino acid sequence and allows easy identification of the protein's primary structure. In human cDNA research to date, more than three million ESTs (Expression Sequence Tags) are publicly available, which presumably covers no less than 80% of all human genes.

EST information is utilized in a variety of ways, for example in analyzing the structure of the human genome, or in predicting exon regions of genomic sequences or their expression profiles. However, most human ESTs have been derived from regions proximal to the cDNA 3′-end, and little information is available from around the mRNA 5′-end. In human cDNAs, the full-length protein sequence of approximately 15,000 corresponding mRNAs have been deduced.

It is possible to identify the mRNA transcription start site on the genomic sequence based on the 5′-end sequence of a full-length cDNA, and to analyze factors involved in the stability of mRNA contained in that sequence, or in the regulation of its expression at the translation stage. Also, since the atg codon, or translation start site, is contained in the 5′-region of a full-length cDNA, translation to proteins will occur in the correct frame. Therefore, it is possible to produce a large amount of the protein encoded by the cDNA, or to analyze the biological activity of the expressed protein by utilizing an appropriate expression system. Thus, analysis of full-length cDNA provides valuable information which complements genome sequencing information. Also, full-length cDNA clones that can be expressed are extremely valuable in empirical analysis of gene function and in industrial application.

Therefore, if a novel human full-length cDNA can be isolated, it can be used for developing medicines for diseases in which its gene is involved. A protein encoded by such a gene can be used as a drug by itself. Thus, obtaining full-length cDNAs encoding novel human proteins is of great significance.

In particular, human secretory proteins or membrane proteins would be useful used as medicines in the same manner as tissue plasminogen activator (TPA), or as target proteins for medicines like membrane receptors. In addition, genes for signal transduction-related proteins (protein kinases, etc.), glycoprotein-related proteins, transcription-related proteins, and such, are genes whose relationships to human diseases have been elucidated. Moreover, genes for disease-related proteins form a gene group rich in genes whose relationships to human diseases have been elucidated.

Isolating novel full-length human cDNA clones, only a few of which have been isolated, is of great significance. The isolation of novel cDNA clones encoding secretory proteins or membrane proteins is especially desired since such proteins would be useful in themselves as medicines, and also their clones would potentially include genes involved in disease. In addition, genes encoding proteins involved in signal transduction, glycoprotein, transcription, or disease, are expected to be useful as target molecules for therapy, or as medicines in themselves. These genes form a gene group predicted to be strongly involved in disease. Thus, identification of full-length cDNA clones encoding these proteins has great significance.

### SUMMARY OF THE INVENTION

An objective of the present invention is to provide polynucleotides encoding novel polypeptides, polypeptides encoded by the polynucleotides, and novel usages of these.

The inventors have developed a method for efficiently cloning, from a cDNA library having a very high fullness-ratio, human full-length cDNAs predicted to be full-length cDNA clones, where that cDNA library is synthesized by an improved method (WO 01/04286) of oligo-capping (K. Maruyama and S. Sugano, Gene, 138: 171-174 (1994); Y. Suzuki et al., Gene, 200: 149-156 (1997)). The nucleotide sequences of cDNA clones whose fullness ratio is high, obtained by this method, were determined mainly from their 5′-end, and, if required, from their 3′-end.

Among the clones obtained, representative clones estimated to be novel and full-length were analyzed for their full-length nucleotide sequence. The determined full-length nucleotide sequences were analyzed using a BLAST homology search of the databases shown below. Homology searches of the present invention were carried out based on full-length cDNA information, including the entire coding region, and thus homology to every part of a polypeptide could be analyzed. Therefore, in the present invention, the reliability of homology searches has been greatly improved.

[1] SwissProt (http://www.ebi.ac.uk/ebi_docsSwissProt_db/swisshome.html),

[2] GenBank (http://www.ncbi.nlm.nih.gov/web/GenBank),

[3] UniGene (Human) (http://www.ncbi.nlm.nih.gov/UniGene),

[4] nr (a protein database, which has been constructed by combining data of coding sequences (CDS) in nucleotide sequences deposited in GenBank, and data of SwissProt, PDB (http://www.rcsb.org/pdb/index.html), PIR (http://pir.georgetown.edu/pirwww/pirhome.shtml), and PRF (http://www.prf.or.jp/en/); overlapping sequences have been removed.), and

[5] RefSeq (http://www.ncbi.nlm.nih.gov/LocusLink/refseq.html).

The gene expression profiles of cDNA clones whose full-length nucleotide sequence had been determined were studied by analyzing the large-scale cDNA database constructed based on the 5′-end nucleotide sequences of cDNAs obtained. The present inventors revealed the usefulness of the genes of the present invention based on these analysis results.

In the present invention, gene functions were revealed by analysis of expression profiles in silico, based on full-length nucleotide sequence information. The expression profiles used in the expression frequency analysis were studied based on databases containing a sufficient amount of fragment sequence data. Expression frequency analysis was carried out by referring, for these expression profiles, to the full-length nucleotide sequences of the many cDNA clones obtained in the present invention. Thus, highly reliable analysis was achieved by referring to the full-length nucleotide sequences of a wide variety of genes in a sufficiently large population for analysis (expression profiles). Namely, the results of expression frequency analysis using the full-length sequences of the present invention more precisely reflect gene expression frequency in tissues and cells from which a certain cDNA library was derived. Thus, the full-length cDNA nucleotide sequence information of the present invention made it possible to achieve highly reliable expression frequency analysis.

The full-length cDNA clones of the present invention were obtained by a method comprising the steps of [1] preparing libraries containing cDNAs with a high fullness ratio using oligo-capping, and [2] assembling 5′-end sequences and selecting those with the highest probability of completeness in length in the cluster formed (there are many clones longer in the 5′-end direction). The use of primers designed based on the 5′- and 3′-end sequences of polynucleotides provided by the present invention enable full-length cDNAs to be readily obtained without using such special techniques. Primers, which are designed for use in obtaining cDNAs capable of being expressed, are not limited to the 5′- and 3′-end sequences of a polynucleotide.

Specifically, the present invention relates to polynucleotides and proteins encoded by the polypeptides as follows.

[1] A polynucleotide selected from the group consisting of:
(a) a polynucleotide comprising a protein-coding region of the nucleotide sequence according to any one of SEQ ID NOs: 1-2188 and SEQ ID NOs: 4377-4683;
(b) a polynucleotide comprising the nucleotide sequence encoding a polypeptide that comprises the amino acid sequence of any one of SEQ ID NOs: 2189-4376 and SEQ ID NOs: 4684-4990;
(c) a polynucleotide comprising a nucleotide sequence encoding a polypeptide, which comprises the amino acid sequence selected from SEQ ID NO: SEQ ID NOs: 2189-4376 and SEQ ID NOs: 4684-4990 wherein one or more amino acids have been substituted, deleted, inserted, and/or added, and which is functionally equivalent to the polypeptide comprising the selected amino acid sequence as described above;
(d) a polynucleotide which hybridizes to a polynucleotide comprising the nucleotide sequence selected from SEQ ID NOs: 1-2188 and SEQ ID NOs: 4377-4683, and which comprises the nucleotide sequence encoding a polypeptide functionally equivalent to a polypeptide encoded by the selected nucleotide sequence as described above;
(e) a polynucleotide comprising a nucleotide sequence encoding a partial amino acid sequence of a polypeptide encoded by the polynucleotides according to any one of (a)-(d);
(f) a polynucleotide comprising a nucleotide sequence having at least 70% identity to the nucleotide sequence of any one of SEQ ID NOs: 1-2188 and SEQ ID NOs: 4377-4683; and
(g) a polynucleotide comprising a nucleotide sequence having at least 90% identity to the nucleotide sequence of any one of SEQ ID NOs: 1-2188 and SEQ ID NOs: 4377-4683.

[2] A polypeptide encoded by the polynucleotide according to [1], or a partial peptide thereof.

[3] An antibody which binds to the polypeptide or the peptide according to [2].

[4] An immunoassay method for the polypeptide or the peptide according to [2], which comprises the steps of:
(a) contacting the polypeptide or the peptide according to [2] with the antibody according to [3]; and
(b) observing the binding between the two.

[5] A vector comprising the polynucleotide according to [1].

[6] A transformant comprising the polynucleotide according to [1] or the vector according to [5].

[7] A transformant which comprises the polynucleotide according to [1] or vector according to [5] in an expressible manner.

[8] A method for producing the polypeptide or the peptide according to [2], which comprises the steps of:
(a) culturing the transformant according to [7]; and
(b) recovering the expression product.

[9] An oligonucleotide comprising 15 or more nucleotides, which comprises the nucleotide sequence according to any one of SEQ ID NOs: 1-2188 and SEQ ID NOs: 4377-4683, or a nucleotide sequence complementary to the complementary strand thereof.

[10] A primer for synthesizing a polynucleotide, which comprises the oligonucleotide according to [9].

[11] A probe for detecting a polynucleotide, which comprises the oligonucleotide according to [9].

[12] A polynucleotide according to any one of:
(a) an antisense polynucleotide comprising a nucleotide sequence complementary to the transcript of the polynucleotide according to [1];
(b) a polynucleotide with the ribozyme activity for specifically cleaving the transcript of the polynucleotide according to [1]; and
(c) a polynucleotide which downregulates the expression of the polynucleotide of [1] due to RNAi activity in a host cell.

[13] A method for detecting the polynucleotide according to [1], which comprises the steps of:
(a) incubating a target polynucleotide with the oligonucleotide according to [9] under conditions ensuring hybridization; and
(b) detecting the hybridization between the target polynucleotide and the oligonucleotide according to [9].

[14] A database of polynucleotides and/or polypeptides, which comprises information on at least one of the nucleotide sequences of SEQ ID NOs: 1-2188 and SEQ ID NOs: 4377-4683 and/or on at least one of the amino acid sequences of SEQ ID NOs: 2189-4376 and SEQ ID NOs: 4684-4990.

Herein, “polynucleotide” is defined as a molecule, such as a DNA or RNA, in which multiple nucleotides are polymerized. There is no limitation as to the number of polymerized nucleotides. If a polymer contains a relatively low number of nucleotides, it is also described as an “oligonucleotide”, which is included in the “polynucleotide” of the present invention. The polynucleotides or oligonucleotides of the present invention can be natural or chemically synthesized. Alternatively, they can be synthesized using a template polynucleotide in an enzymatic reaction such as PCR. Furthermore, the polynucleotides of the present invention may be modified chemically. Single-stranded and double-stranded polynucleotides are included in the present invention. In this specification, especially in the claims, when the polynucleotides are described merely as “polynucleotide”, it means not only single-stranded polynucleotides but also double-stranded polynucleotides. When it means a double-stranded polynucleotide, the nucleotide sequence of only one chain is indicated. However, based on the nucleotide sequence of a sense chain, the nucleotide sequence of the corresponding complementary strand is essentially determined.

All the cDNAs provided by the present invention are full-length cDNAs. “Full-length cDNAs” herein means cDNAs containing the ATG codon, which is the start point of translation therein. Untranslated regions upstream and downstream of the protein-coding region are both naturally contained in natural mRNAs and are not essential. It is preferable that the full-length cDNAs of the present invention contain a stop codon.

### BRIEF DESCRIPTION OF THE DRAWINGS

FIG. 1 shows the restriction map of the vector pME18SFL3.

### DETAILED DESCRIPTION OF THE INVENTION

All of the clones (2495 clones) of the present invention are novel and encode full-length polypeptides. Further, all of the clones are cDNAs with a high fullness ratio, and which were obtained by oligo-capping method. None of the clones are identical to any known human mRNAs (namely, they are novel clones) selected by searching 5′-end sequences and mRNA sequences with the annotation of “complete cds” in the GenBank and UniGene databases using BLAST homology [S. F. Altschul, W. Gish, W. Miller, E. W. Myers & D. J. Lipman, J. Mol. Biol., 215: 403-410 (1990); W. Gish & D. J. States, Nature Genet., 3: 266-272 (1993)]. They are also clones that were assumed to have a higher fullness ratio among members in assembled clusters. Most of the clones with a high fullness ratio in a cluster had nucleotide sequences longer in the 5′-end direction.

All of the full-length cDNAs of the present invention can be synthesized by a method such as PCR (Current protocols in Molecular Biology edit. Ausubel et al. (1987) Publish. John Wiley & Sons Section 6.1-6.4) using primer sets designed based on 5′-end and 3′-end sequences, or using primer sets of primers designed based on 5′-end sequences and a primer of oligo dT sequence corresponding to poly A sequence. Table 1 contains the clone names of 2,495 full-length cDNA clones of the present invention, SEQ ID NOs of the full-length nucleotide sequences, CDS portions deduced from the full-length nucleotide, sequences, and SEQ ID NOs of the translated amino acids. The positions of CDS are shown according to the rules set out in “DDBJ/EMBL/GenBank Feature Table Definition” (http://www.ncbi.nlm.nih.gov/collab/FT/index.html). The start position number corresponds to the first letter of “ATG”, the nucleotide triplet encoding methionine; the termination position number corresponds to the third letter of the stop codon. These are indicated by flanking with the mark “..”. However, in clones without a stop codon, the termination position is indicated by the mark “>”, according to the above rules.

TABLE 1

| | | | |
|---|---|---|---|
| 3NB692002685 | 1 | 777 . . . 1496 | 2189 |
| 3NB692002806 | 2 | 33 . . . 674 | 2190 |
| 3NB692008729 | 3 | 455 . . . 1189 | 2191 |
| ACTVT2000380 | 4 | 593 . . . 919 | 2192 |
| ADIPS2000088 | 5 | 59 . . . 1486 | 2193 |
| ADRGL2000172 | 6 | 237 . . . 638 | 2194 |
| ADRGL2003329 | 7 | 640 . . . 1014 | 2195 |
| ADRGL2009146 | 8 | 313 . . . 732 | 2196 |
| ADRGL2009691 | 9 | 294 . . . 767 | 2197 |
| ADRGL2009755 | 10 | 87 . . . 1265 | 2198 |
| ADRGL2012038 | 11 | 342 . . . 686 | 2199 |
| ADRGL2012179 | 12 | 254 . . . 625 | 2200 |
| ASTRO1000009 | 13 | 458 . . . 1552 | 2201 |
| ASTRO2002842 | 14 | 150 . . . 1490 | 2202 |
| ASTRO2003960 | 15 | 422 . . . 895 | 2203 |
| ASTRO2014923 | 16 | 65 . . . 1357 | 2204 |
| ASTRO2018373 | 17 | 123 . . . 512 | 2205 |
| ASTRO3000172 | 18 | 2582 . . . 2890 | 2206 |
| ASTRO3000177 | 19 | 2311 . . . 3978 | 2207 |
| ASTRO3000301 | 20 | 125 . . . 3832 | 2208 |
| ASTRO3000482 | 21 | 90 . . . 557 | 2209 |
| BLADE1000176 | 22 | 1787 . . . 2101 | 2210 |
| BLADE2001371 | 23 | 1009 . . . 1323 | 2211 |
| BLADE2001987 | 24 | 186 . . . 548 | 2212 |
| BLADE2002073 | 25 | 481 . . . 801 | 2213 |
| BLADE2002782 | 26 | 1341 . . . 1667 | 2214 |
| BLADE2002947 | 27 | 7 . . . 336 | 2215 |
| BLADE2003474 | 28 | 967 . . . 1272 | 2216 |
| BLADE2004089 | 29 | 60 . . . >2486 | 2217 |
| BLADE2004462 | 30 | 96 . . . 512 | 2218 |
| BLADE2004670 | 31 | 2037 . . . >2574 | 2219 |
| BLADE2005036 | 32 | 1293 . . . 1838 | 2220 |
| BLADE2005459 | 33 | 306 . . . 977 | 2221 |
| BLADE2007666 | 34 | 2289 . . . 2612 | 2222 |
| BLADE2007958 | 35 | 1147 . . . 1530 | 2223 |
| BLADE2008281 | 36 | 26 . . . 370 | 2224 |
| BLADE2008398 | 37 | 2742 . . . 3422 | 2225 |
| BLADE2008539 | 38 | 2857 . . . 4245 | 2226 |
| BNGH42003570 | 39 | 98 . . . 802 | 2227 |
| BNGH42007788 | 40 | 472 . . . 1782 | 2228 |
| BRACE1000186 | 41 | 333 . . . 821 | 2229 |
| BRACE1000258 | 42 | 10 . . . 2538 | 2230 |
| BRACE1000533 | 43 | 1483 . . . 1908 | 2231 |
| BRACE1000572 | 44 | 16 . . . 3435 | 2232 |
| BRACE2003639 | 45 | 481 . . . 1065 | 2233 |
| BRACE2005457 | 46 | 128 . . . 1237 | 2234 |
| BRACE2006319 | 47 | 603 . . . 1517 | 2235 |
| BRACE2008594 | 48 | 1221 . . . 1871 | 2236 |
| BRACE2010489 | 49 | 262 . . . >1693 | 2237 |
| BRACE2011747 | 50 | 983 . . . 1438 | 2238 |
| BRACE2014306 | 51 | 32 . . . 1402 | 2239 |
| BRACE2014475 | 52 | 598 . . . 906 | 2240 |
| BRACE2014657 | 53 | 404 . . . 814 | 2241 |
| BRACE2015058 | 54 | 670 . . . 1116 | 2242 |
| BRACE2015314 | 55 | 203 . . . 1822 | 2243 |
| BRACE2016981 | 56 | 51 . . . 1355 | 2244 |
| BRACE2018762 | 57 | 654 . . . 1739 | 2245 |
| BRACE2024627 | 58 | 438 . . . 950 | 2246 |
| BRACE2026836 | 59 | 134 . . . 1087 | 2247 |
| BRACE2027258 | 60 | 164 . . . >1851 | 2248 |
| BRACE2027970 | 61 | 1538 . . . 1990 | 2249 |
| BRACE2028970 | 62 | 2257 . . . 2685 | 2250 |
| BRACE2029112 | 63 | 832 . . . 1191 | 2251 |
| BRACE2029849 | 64 | 1039 . . . 1341 | 2252 |
| BRACE2030326 | 65 | 344 . . . 688 | 2253 |
| BRACE2030341 | 66 | 1209 . . . 1694 | 2254 |
| BRACE2030884 | 67 | 1248 . . . 1601 | 2255 |
| BRACE2031154 | 68 | 2 . . . 367 | 2256 |
| BRACE2031389 | 69 | 690 . . . 1271 | 2257 |
| BRACE2031527 | 70 | 367 . . . 684 | 2258 |
| BRACE2031531 | 71 | 120 . . . 635 | 2259 |
| BRACE2031899 | 72 | 46 . . . 381 | 2260 |
| BRACE2032044 | 73 | 403 . . . 732 | 2261 |
| BRACE2032329 | 74 | 1330 . . . 1761 | 2262 |
| BRACE2032385 | 75 | 20 . . . 613 | 2263 |
| BRACE2032538 | 76 | 189 . . . 515 | 2264 |
| BRACE2032823 | 77 | 1910 . . . 2212 | 2265 |
| BRACE2033720 | 78 | 28 . . . 537 | 2266 |
| BRACE2035381 | 79 | 1310 . . . 2758 | 2267 |
| BRACE2035441 | 80 | 98 . . . 1660 | 2268 |
| BRACE2036005 | 81 | 1584 . . . 1913 | 2269 |
| BRACE2036096 | 82 | 1173 . . . 1484 | 2270 |
| BRACE2036830 | 83 | 391 . . . 702 | 2271 |
| BRACE2036834 | 84 | 1436 . . . 1894 | 2272 |
| BRACE2037847 | 85 | 122 . . . 616 | 2273 |
| BRACE2038114 | 86 | 171 . . . 536 | 2274 |
| BRACE2038329 | 87 | 335 . . . 928 | 2275 |
| BRACE2038551 | 88 | 1913 . . . 2239 | 2276 |
| BRACE2039249 | 89 | 1085 . . . 1726 | 2277 |
| BRACE2039327 | 90 | 361 . . . 1419 | 2278 |
| BRACE2039475 | 91 | 307 . . . 753 | 2279 |
| BRACE2039734 | 92 | 16 . . . >1730 | 2280 |
| BRACE2040138 | 93 | 797 . . . 1216 | 2281 |
| BRACE2040325 | 94 | 356 . . . 757 | 2282 |
| BRACE2041009 | 95 | 29 . . . 1390 | 2283 |
| BRACE2041200 | 96 | 304 . . . 945 | 2284 |
| BRACE2041264 | 97 | 530 . . . 1105 | 2285 |
| BRACE2042550 | 98 | 15 . . . 1007 | 2286 |
| BRACE2043142 | 99 | 156 . . . 908 | 2287 |
| BRACE2043248 | 100 | 1099 . . . 1581 | 2288 |
| BRACE2043349 | 101 | 202 . . . 531 | 2289 |
| BRACE2043665 | 102 | 858 . . . 1565 | 2290 |
| BRACE2044286 | 103 | 125 . . . 2011 | 2291 |
| BRACE2044816 | 104 | 188 . . . 670 | 2292 |
| BRACE2044949 | 105 | 24 . . . 725 | 2293 |
| BRACE2045300 | 106 | 1174 . . . 1863 | 2294 |
| BRACE2045428 | 107 | 209 . . . 625 | 2295 |
| BRACE2045596 | 108 | 906 . . . 1583 | 2296 |
| BRACE2045772 | 109 | 31 . . . 777 | 2297 |
| BRACE2045947 | 110 | 499 . . . 1164 | 2298 |
| BRACE2045954 | 111 | 1558 . . . 1863 | 2299 |
| BRACE2046251 | 112 | 527 . . . 1360 | 2300 |
| BRACE2046295 | 113 | 436 . . . 1428 | 2301 |
| BRACE2047011 | 114 | 1453 . . . 1794 | 2302 |
| BRACE2047350 | 115 | 2840 . . . 3487 | 2303 |
| BRACE2047377 | 116 | 133 . . . 456 | 2304 |
| BRACE2047385 | 117 | 75 . . . 458 | 2305 |
| BRACE3000071 | 118 | 1729 . . . 2124 | 2306 |
| BRACE3000697 | 119 | 131 . . . 703 | 2307 |
| BRACE3000787 | 120 | 2208 . . . 2750 | 2308 |
| BRACE3000840 | 121 | 564 . . . 3611 | 2309 |
| BRACE3000973 | 122 | 887 . . . 1666 | 2310 |
| BRACE3001002 | 123 | 292 . . . 624 | 2311 |
| BRACE3001217 | 124 | 2924 . . . 3241 | 2312 |
| BRACE3001391 | 125 | 1803 . . . 3506 | 2313 |
| BRACE3001595 | 126 | 8 . . . 952 | 2314 |
| BRACE3001754 | 127 | 586 . . . 1188 | 2315 |
| BRACE3002298 | 128 | 67 . . . 375 | 2316 |
| BRACE3002390 | 129 | 136 . . . 537 | 2317 |
| BRACE3002508 | 130 | 3430 . . . 4104 | 2318 |
| BRACE3003004 | 131 | 692 . . . 1027 | 2319 |
| BRACE3003192 | 132 | 1044 . . . 3245 | 2320 |
| BRACE3003595 | 133 | 1654 . . . 4179 | 2321 |
| BRACE3003698 | 134 | 506 . . . 847 | 2322 |
| BRACE3004058 | 135 | 2267 . . . 3040 | 2323 |
| BRACE3004113 | 136 | 1887 . . . 2189 | 2324 |
| BRACE3004150 | 137 | 1537 . . . 2886 | 2325 |
| BRACE3004358 | 138 | 222 . . . 551 | 2326 |
| BRACE3004435 | 139 | 2782 . . . 3240 | 2327 |
| BRACE3004772 | 140 | 888 . . . 1577 | 2328 |
| BRACE3004783 | 141 | 132 . . . 644 | 2329 |
| BRACE3004843 | 142 | 183 . . . 500 | 2330 |
| BRACE3004880 | 143 | 885 . . . 1655 | 2331 |
| BRACE3005145 | 144 | 717 . . . 1484 | 2332 |
| BRACE3005225 | 145 | 76 . . . 495 | 2333 |
| BRACE3005430 | 146 | 3120 . . . 3524 | 2334 |
| BRACE3005499 | 147 | 205 . . . 531 | 2335 |
| BRACE3006185 | 148 | 220 . . . 648 | 2336 |
| BRACE3006226 | 149 | 580 . . . 951 | 2337 |
| BRACE3006462 | 150 | 1851 . . . 2213 | 2338 |
| BRACE3006872 | 151 | 1136 . . . 1957 | 2339 |
| BRACE3007322 | 152 | 2918 . . . >3227 | 2340 |
| BRACE3007472 | 153 | 190 . . . 573 | 2341 |
| BRACE3007480 | 154 | 93 . . . 962 | 2342 |
| BRACE3007559 | 155 | 1236 . . . 1592 | 2343 |
| BRACE3007625 | 156 | 36 . . . 3053 | 2344 |
| BRACE3007642 | 157 | 1857 . . . 2294 | 2345 |
| BRACE3007767 | 158 | 237 . . . 758 | 2346 |
| BRACE3008036 | 159 | 162 . . . 512 | 2347 |
| BRACE3008092 | 160 | 3356 . . . 3724 | 2348 |
| BRACE3008137 | 161 | 208 . . . 3699 | 2349 |
| BRACE3008384 | 162 | 123 . . . 1043 | 2350 |
| BRACE3008720 | 163 | 3014 . . . 3550 | 2351 |
| BRACE3008772 | 164 | 3900 . . . 4331 | 2352 |
| BRACE3009090 | 165 | 70 . . . 585 | 2353 |
| BRACE3009237 | 166 | 406 . . . 714 | 2354 |
| BRACE3009297 | 167 | 2724 . . . 3107 | 2355 |
| BRACE3009377 | 168 | 1359 . . . 1790 | 2356 |
| BRACE3009574 | 169 | 180 . . . 527 | 2357 |
| BRACE3009701 | 170 | 1011 . . . 1550 | 2358 |
| BRACE3009708 | 171 | 105 . . . 2867 | 2359 |
| BRACE3009724 | 172 | 2401 . . . 3282 | 2360 |
| BRACE3009747 | 173 | 2650 . . . 3171 | 2361 |
| BRACE3010397 | 174 | 1848 . . . 2660 | 2362 |
| BRACE3010428 | 175 | 269 . . . 628 | 2363 |
| BRACE3011271 | 176 | 689 . . . 1417 | 2364 |
| BRACE3011421 | 177 | 372 . . . 3209 | 2365 |
| BRACE3011505 | 178 | 688 . . . 1035 | 2366 |
| BRACE3012364 | 179 | 1980 . . . 2633 | 2367 |
| BRACE3012930 | 180 | 1128 . . . 1718 | 2368 |
| BRACE3013119 | 181 | 66 . . . 395 | 2369 |
| BRACE3013576 | 182 | 2120 . . . 2971 | 2370 |
| BRACE3013740 | 183 | 118 . . . 447 | 2371 |
| BRACE3013780 | 184 | 6 . . . 1124 | 2372 |
| BRACE3014005 | 185 | 1460 . . . 2374 | 2373 |
| BRACE3014068 | 186 | 29 . . . 373 | 2374 |
| BRACE3014231 | 187 | 928 . . . 1347 | 2375 |
| BRACE3014317 | 188 | 44 . . . 550 | 2376 |
| BRACE3014807 | 189 | 144 . . . 1202 | 2377 |
| BRACE3015027 | 190 | 1 . . . 1656 | 2378 |
| BRACE3015121 | 191 | 3019 . . . 4404 | 2379 |
| BRACE3015262 | 192 | 27 . . . 2195 | 2380 |
| BRACE3015521 | 193 | 2117 . . . 2827 | 2381 |
| BRACE3015894 | 194 | 285 . . . 773 | 2382 |
| BRACE3016884 | 195 | 303 . . . 2633 | 2383 |
| BRACE3018308 | 196 | 106 . . . 612 | 2384 |
| BRACE3018963 | 197 | 332 . . . 676 | 2385 |
| BRACE3019055 | 198 | 1065 . . . 1940 | 2386 |
| BRACE3019084 | 199 | 1644 . . . 2369 | 2387 |
| BRACE3020194 | 200 | 1727 . . . 2161 | 2388 |
| BRACE3020286 | 201 | 85 . . . 426 | 2389 |
| BRACE3020594 | 202 | 250 . . . 624 | 2390 |
| BRACE3022769 | 203 | 299 . . . 790 | 2391 |
| BRACE3023912 | 204 | 12 . . . 539 | 2392 |
| BRACE3024073 | 205 | 86 . . . 655 | 2393 |
| BRACE3024659 | 206 | 14 . . . 586 | 2394 |
| BRACE3024662 | 207 | 401 . . . 925 | 2395 |
| BRACE3025153 | 208 | 98 . . . 523 | 2396 |
| BRACE3025457 | 209 | 1204 . . . 2541 | 2397 |
| BRACE3025531 | 210 | 1319 . . . 2338 | 2398 |
| BRACE3025630 | 211 | 1329 . . . 1919 | 2399 |
| BRACE3026008 | 212 | 250 . . . 558 | 2400 |
| BRACE3026075 | 213 | 130 . . . 483 | 2401 |
| BRACE3026735 | 214 | 324 . . . 635 | 2402 |
| BRACE3027242 | 215 | 273 . . . 791 | 2403 |
| BRACE3027326 | 216 | 237 . . . 2372 | 2404 |
| BRACE3027478 | 217 | 32 . . . 529 | 2405 |
| BRACE3030103 | 218 | 320 . . . 682 | 2406 |
| BRACE3031838 | 219 | 1661 . . . 1981 | 2407 |
| BRACE3032983 | 220 | 155 . . . 484 | 2408 |
| BRACE3040856 | 221 | 187 . . . 585 | 2409 |
| BRACE3045033 | 222 | 24 . . . 566 | 2410 |
| BRALZ2011796 | 223 | 132 . . . 1361 | 2411 |
| BRALZ2012183 | 224 | 2172 . . . 2741 | 2412 |
| BRALZ2012848 | 225 | 218 . . . 754 | 2413 |
| BRALZ2014484 | 226 | 80 . . . 1411 | 2414 |
| BRALZ2016085 | 227 | 217 . . . >1672 | 2415 |
| BRALZ2016498 | 228 | 402 . . . 893 | 2416 |
| BRALZ2017359 | 229 | 47 . . . 973 | 2417 |
| BRAMY2001473 | 230 | 112 . . . 1701 | 2418 |
| BRAMY2003008 | 231 | 236 . . . 961 | 2419 |
| BRAMY2004771 | 232 | 240 . . . 2108 | 2420 |
| BRAMY2005052 | 233 | 211 . . . 1434 | 2421 |
| BRAMY2017528 | 234 | 447 . . . 1076 | 2422 |
| BRAMY2019300 | 235 | 338 . . . 2110 | 2423 |
| BRAMY2019963 | 236 | 206 . . . 640 | 2424 |
| BRAMY2019985 | 237 | 271 . . . 573 | 2425 |
| BRAMY2020058 | 238 | 1537 . . . 1962 | 2426 |
| BRAMY2020270 | 239 | 98 . . . 976 | 2427 |
| BRAMY2021498 | 240 | 71 . . . 1870 | 2428 |
| BRAMY2028856 | 241 | 16 . . . 333 | 2429 |
| BRAMY2028914 | 242 | 34 . . . 534 | 2430 |
| BRAMY2029602 | 243 | 216 . . . 731 | 2431 |
| BRAMY2030098 | 244 | 960 . . . 1298 | 2432 |
| BRAMY2030109 | 245 | 271 . . . 1296 | 2433 |
| BRAMY2030702 | 246 | 1008 . . . 1316 | 2434 |
| BRAMY2030703 | 247 | 132 . . . 611 | 2435 |
| BRAMY2030799 | 248 | 85 . . . 480 | 2436 |
| BRAMY2031317 | 249 | 313 . . . 2334 | 2437 |
| BRAMY2031377 | 250 | 214 . . . 642 | 2438 |
| BRAMY2031442 | 251 | 1492 . . . 1854 | 2439 |
| BRAMY2032014 | 252 | 1227 . . . 2054 | 2440 |
| BRAMY2032242 | 253 | 404 . . . 1150 | 2441 |
| BRAMY2032317 | 254 | 1649 . . . 1987 | 2442 |
| BRAMY2033003 | 255 | 736 . . . 1068 | 2443 |
| BRAMY2033116 | 256 | 1008 . . . 1721 | 2444 |
| BRAMY2033267 | 257 | 609 . . . 923 | 2445 |
| BRAMY2033594 | 258 | 2121 . . . 2447 | 2446 |
| BRAMY2034185 | 259 | 47 . . . 400 | 2447 |
| BRAMY2034920 | 260 | 222 . . . 620 | 2448 |
| BRAMY2034993 | 261 | 1613 . . . 2041 | 2449 |
| BRAMY2036387 | 262 | 1858 . . . 2160 | 2450 |
| BRAMY2036396 | 263 | 837 . . . 1169 | 2451 |
| BRAMY2036567 | 264 | 859 . . . 1338 | 2452 |
| BRAMY2036699 | 265 | 60 . . . 374 | 2453 |
| BRAMY2036913 | 266 | 86 . . . 433 | 2454 |
| BRAMY2037823 | 267 | 2163 . . . 2549 | 2455 |
| BRAMY2038100 | 268 | 515 . . . 997 | 2456 |
| BRAMY2038484 | 269 | 95 . . . 709 | 2457 |
| BRAMY2038846 | 270 | 1709 . . . 2356 | 2458 |
| BRAMY2038904 | 271 | 118 . . . 2274 | 2459 |
| BRAMY2039872 | 272 | 663 . . . 1148 | 2460 |
| BRAMY2040478 | 273 | 1754 . . . 2083 | 2461 |
| BRAMY2040592 | 274 | 954 . . . 1943 | 2462 |
| BRAMY2041261 | 275 | 124 . . . 441 | 2463 |
| BRAMY2041378 | 276 | 199 . . . 831 | 2464 |
| BRAMY2041542 | 277 | 2098 . . . 2412 | 2465 |
| BRAMY2042612 | 278 | 1319 . . . 1621 | 2466 |
| BRAMY2042641 | 279 | 271 . . . 672 | 2467 |
| BRAMY2042760 | 280 | 1306 . . . 2115 | 2468 |
| BRAMY2042918 | 281 | 956 . . . 1354 | 2469 |
| BRAMY2044078 | 282 | 123 . . . 569 | 2470 |
| BRAMY2044246 | 283 | 83 . . . 436 | 2471 |
| BRAMY2045036 | 284 | 2746 . . . 3069 | 2472 |
| BRAMY2046478 | 285 | 51 . . . 359 | 2473 |
| BRAMY2046742 | 286 | 819 . . . 1148 | 2474 |
| BRAMY2046989 | 287 | 47 . . . 1594 | 2475 |
| BRAMY2047169 | 288 | 1622 . . . 2083 | 2476 |
| BRAMY2047420 | 289 | 573 . . . 1055 | 2477 |
| BRAMY2047676 | 290 | 30 . . . 677 | 2478 |
| BRAMY2047746 | 291 | 109 . . . 1566 | 2479 |
| BRAMY2047751 | 292 | 859 . . . 2040 | 2480 |
| BRAMY2047765 | 293 | 2247 . . . 2549 | 2481 |
| BRAMY2047884 | 294 | 873 . . . 1199 | 2482 |
| BRAMY3000206 | 295 | 1072 . . . 1689 | 2483 |
| BRAMY3000213 | 296 | 2109 . . . 2888 | 2484 |
| BRAMY3001401 | 297 | 109 . . . 594 | 2485 |
| BRAMY3001794 | 298 | 51 . . . 1322 | 2486 |
| BRAMY3002312 | 299 | 58 . . . 372 | 2487 |
| BRAMY3002620 | 300 | 113 . . . 2752 | 2488 |
| BRAMY3002803 | 301 | 913 . . . 2823 | 2489 |
| BRAMY3002805 | 302 | 2036 . . . 2371 | 2490 |
| BRAMY3004224 | 303 | 414 . . . 1526 | 2491 |
| BRAMY3004672 | 304 | 1926 . . . 2402 | 2492 |
| BRAMY3004900 | 305 | 165 . . . 635 | 2493 |
| BRAMY3004919 | 306 | 539 . . . 3298 | 2494 |
| BRAMY3005091 | 307 | 592 . . . 1311 | 2495 |
| BRAMY3005932 | 308 | 2455 . . . 3075 | 2496 |
| BRAMY3006297 | 309 | 76 . . . 456 | 2497 |
| BRAMY3007206 | 310 | 720 . . . 1901 | 2498 |
| BRAMY3007609 | 311 | 744 . . . 1745 | 2499 |
| BRAMY3008466 | 312 | 1458 . . . 2342 | 2500 |
| BRAMY3008505 | 313 | 2733 . . . 3125 | 2501 |
| BRAMY3008650 | 314 | 656 . . . 958 | 2502 |
| BRAMY3009811 | 315 | 1842 . . . 2321 | 2503 |
| BRAMY3010411 | 316 | 707 . . . 1066 | 2504 |
| BRAMY4000095 | 317 | 736 . . . 2487 | 2505 |
| BRAMY4000229 | 318 | 1214 . . . 1957 | 2506 |
| BRAMY4000277 | 319 | 1027 . . . 2064 | 2507 |
| BRASW1000053 | 320 | 1315 . . . 1632 | 2508 |
| BRASW1000125 | 321 | 1099 . . . 1443 | 2509 |
| BRAWH1000127 | 322 | 622 . . . 1353 | 2510 |
| BRAWH2001395 | 323 | 696 . . . 1130 | 2511 |
| BRAWH2001671 | 324 | 1559 . . . 2077 | 2512 |
| BRAWH2001940 | 325 | 172 . . . 2208 | 2513 |
| BRAWH2001973 | 326 | 133 . . . 498 | 2514 |
| BRAWH2002560 | 327 | 176 . . . 2476 | 2515 |
| BRAWH2002761 | 328 | 555 . . . 1079 | 2516 |
| BRAWH2005315 | 329 | 161 . . . 1615 | 2517 |
| BRAWH2007658 | 330 | 45 . . . 1148 | 2518 |
| BRAWH2010000 | 331 | 275 . . . 2155 | 2519 |
| BRAWH2010084 | 332 | 1682 . . . 2191 | 2520 |
| BRAWH2010536 | 333 | 448 . . . 1284 | 2521 |
| BRAWH2012162 | 334 | 512 . . . 2068 | 2522 |
| BRAWH2012326 | 335 | 115 . . . 426 | 2523 |
| BRAWH2013294 | 336 | 762 . . . 1148 | 2524 |
| BRAWH2013871 | 337 | 756 . . . 1151 | 2525 |
| BRAWH2014414 | 338 | 266 . . . 2794 | 2526 |
| BRAWH2014645 | 339 | 940 . . . 1632 | 2527 |
| BRAWH2014662 | 340 | 285 . . . 1706 | 2528 |
| BRAWH2014876 | 341 | 148 . . . 840 | 2529 |
| BRAWH2014954 | 342 | 377 . . . >2183 | 2530 |
| BRAWH2016221 | 343 | 1235 . . . 1891 | 2531 |
| BRAWH2016439 | 344 | 2332 . . . 2754 | 2532 |
| BRAWH2016702 | 345 | 1328 . . . 2347 | 2533 |
| BRAWH2016724 | 346 | 846 . . . 1292 | 2534 |
| BRAWH3000078 | 347 | 290 . . . 805 | 2535 |
| BRAWH3000100 | 348 | 3063 . . . 5708 | 2536 |
| BRAWH3000314 | 349 | 1322 . . . 3064 | 2537 |
| BRAWH3000345 | 350 | 1135 . . . 1452 | 2538 |
| BRAWH3000491 | 351 | 590 . . . 913 | 2539 |
| BRAWH3001326 | 352 | 73 . . . 987 | 2540 |
| BRAWH3001475 | 353 | 75 . . . 377 | 2541 |
| BRAWH3001891 | 354 | 11 . . . 1141 | 2542 |
| BRAWH3002574 | 355 | 1585 . . . 2217 | 2543 |
| BRAWH3002600 | 356 | 104 . . . 1795 | 2544 |
| BRAWH3002819 | 357 | 2589 . . . 3233 | 2545 |
| BRAWH3002821 | 358 | 383 . . . 3064 | 2546 |
| BRAWH3003522 | 359 | 69 . . . 554 | 2547 |
| BRAWH3003555 | 360 | 1048 . . . 1545 | 2548 |
| BRAWH3003727 | 361 | 1386 . . . 1694 | 2549 |
| BRAWH3003801 | 362 | 2985 . . . 3470 | 2550 |
| BRAWH3003992 | 363 | 2123 . . . 2677 | 2551 |
| BRAWH3004453 | 364 | 1891 . . . 2346 | 2552 |
| BRAWH3004666 | 365 | 1158 . . . 1562 | 2553 |
| BRAWH3005132 | 366 | 2935 . . . 3333 | 2554 |
| BRAWH3005422 | 367 | 14 . . . 535 | 2555 |
| BRAWH3005912 | 368 | 1384 . . . 3516 | 2556 |
| BRAWH3005981 | 369 | 160 . . . 2685 | 2557 |
| BRAWH3006548 | 370 | 2431 . . . 2760 | 2558 |
| BRAWH3006792 | 371 | 681 . . . 1313 | 2559 |
| BRAWH3007221 | 372 | 854 . . . 1345 | 2560 |
| BRAWH3007506 | 373 | 986 . . . 1459 | 2561 |
| BRAWH3007592 | 374 | 2933 . . . 3331 | 2562 |
| BRAWH3007726 | 375 | 352 . . . 780 | 2563 |
| BRAWH3007783 | 376 | 779 . . . 1435 | 2564 |
| BRAWH3008341 | 377 | 1608 . . . 2198 | 2565 |
| BRAWH3008634 | 378 | 1099 . . . 1515 | 2566 |
| BRAWH3008697 | 379 | 605 . . . 1294 | 2567 |
| BRAWH3008931 | 380 | 11 . . . 469 | 2568 |
| BRAWH3009297 | 381 | 1581 . . . 2141 | 2569 |
| BRCAN2002562 | 382 | 215 . . . 880 | 2570 |
| BRCAN2002856 | 383 | 380 . . . 1114 | 2571 |
| BRCAN2002944 | 384 | 22 . . . 609 | 2572 |
| BRCAN2002948 | 385 | 42 . . . 431 | 2573 |
| BRCAN2003703 | 386 | 579 . . . 1157 | 2574 |
| BRCAN2003746 | 387 | 1773 . . . 2543 | 2575 |
| BRCAN2003987 | 388 | 142 . . . 447 | 2576 |
| BRCAN2004355 | 389 | 722 . . . 1024 | 2577 |
| BRCAN2005436 | 390 | 45 . . . 764 | 2578 |
| BRCAN2006063 | 391 | 547 . . . 1278 | 2579 |
| BRCAN2006290 | 392 | 117 . . . 419 | 2580 |
| BRCAN2006297 | 393 | 490 . . . 999 | 2581 |
| BRCAN2006450 | 394 | 3 . . . 320 | 2582 |
| BRCAN2007144 | 395 | 94 . . . 636 | 2583 |
| BRCAN2007409 | 396 | 2280 . . . >2620 | 2584 |
| BRCAN2007426 | 397 | 62 . . . 601 | 2585 |
| BRCAN2008528 | 398 | 311 . . . 1231 | 2586 |
| BRCAN2009203 | 399 | 1077 . . . 2408 | 2587 |
| BRCAN2009432 | 400 | 95 . . . 646 | 2588 |
| BRCAN2010376 | 401 | 227 . . . 571 | 2589 |
| BRCAN2011254 | 402 | 252 . . . 557 | 2590 |
| BRCAN2011602 | 403 | 224 . . . 748 | 2591 |
| BRCAN2012355 | 404 | 467 . . . 1009 | 2592 |
| BRCAN2012481 | 405 | 240 . . . 566 | 2593 |
| BRCAN2013655 | 406 | 268 . . . 606 | 2594 |
| BRCAN2013660 | 407 | 216 . . . 641 | 2595 |
| BRCAN2014143 | 408 | 15 . . . 584 | 2596 |
| BRCAN2014602 | 409 | 94 . . . 870 | 2597 |
| BRCAN2014881 | 410 | 279 . . . 758 | 2598 |
| BRCAN2015371 | 411 | 49 . . . 432 | 2599 |
| BRCAN2015464 | 412 | 491 . . . 955 | 2600 |
| BRCAN2016433 | 413 | 1679 . . . 1993 | 2601 |
| BRCAN2016619 | 414 | 77 . . . 2743 | 2602 |
| BRCAN2017442 | 415 | 82 . . . 492 | 2603 |
| BRCAN2017717 | 416 | 63 . . . 377 | 2604 |
| BRCAN2017905 | 417 | 38 . . . 532 | 2605 |
| BRCAN2018935 | 418 | 156 . . . 719 | 2606 |
| BRCAN2019387 | 419 | 9 . . . 314 | 2607 |
| BRCAN2020710 | 420 | 1738 . . . 2043 | 2608 |
| BRCAN2021028 | 421 | 94 . . . 1734 | 2609 |
| BRCAN2024451 | 422 | 184 . . . 1227 | 2610 |
| BRCAN2024563 | 423 | 1 . . . 384 | 2611 |
| BRCAN2025712 | 424 | 780 . . . 1187 | 2612 |
| BRCAN2028355 | 425 | 78 . . . 1805 | 2613 |
| BRCOC2000670 | 426 | 326 . . . 628 | 2614 |
| BRCOC2001505 | 427 | 1470 . . . 1904 | 2615 |
| BRCOC2003213 | 428 | 1821 . . . 3143 | 2616 |
| BRCOC2007034 | 429 | 404 . . . 931 | 2617 |
| BRCOC2014033 | 430 | 359 . . . 709 | 2618 |
| BRCOC2016525 | 431 | 153 . . . 1997 | 2619 |
| BRCOC2019934 | 432 | 5 . . . 385 | 2620 |
| BRCOC2020142 | 433 | 741 . . . 1130 | 2621 |
| BRHIP2000691 | 434 | 1545 . . . 1910 | 2622 |
| BRHIP2000819 | 435 | 1030 . . . 1611 | 2623 |
| BRHIP2000826 | 436 | 137 . . . 442 | 2624 |
| BRHIP2000920 | 437 | 836 . . . 1567 | 2625 |
| BRHIP2001074 | 438 | 553 . . . 1581 | 2626 |
| BRHIP2001805 | 439 | 70 . . . 528 | 2627 |
| BRHIP2001927 | 440 | 2 . . . 340 | 2628 |
| BRHIP2002122 | 441 | 530 . . . 1153 | 2629 |
| BRHIP2002172 | 442 | 673 . . . 2031 | 2630 |
| BRHIP2002346 | 443 | 159 . . . 1097 | 2631 |
| BRHIP2003242 | 444 | 72 . . . 575 | 2632 |
| BRHIP2003786 | 445 | 199 . . . 2013 | 2633 |
| BRHIP2003917 | 446 | 1870 . . . 2316 | 2634 |
| BRHIP2004312 | 447 | 1748 . . . 2200 | 2635 |
| BRHIP2004359 | 448 | 663 . . . 2027 | 2636 |
| BRHIP2004814 | 449 | 642 . . . 1778 | 2637 |
| BRHIP2004883 | 450 | 221 . . . 526 | 2638 |
| BRHIP2005236 | 451 | 709 . . . 1860 | 2639 |
| BRHIP2005354 | 452 | 569 . . . 1057 | 2640 |
| BRHIP2005600 | 453 | 492 . . . 1187 | 2641 |
| BRHIP2005719 | 454 | 445 . . . 909 | 2642 |
| BRHIP2005752 | 455 | 876 . . . 1919 | 2643 |
| BRHIP2005932 | 456 | 212 . . . 652 | 2644 |
| BRHIP2006800 | 457 | 178 . . . 774 | 2645 |
| BRHIP2007616 | 458 | 136 . . . 1614 | 2646 |
| BRHIP2007741 | 459 | 811 . . . 1122 | 2647 |
| BRHIP2009340 | 460 | 72 . . . 374 | 2648 |
| BRHIP2009414 | 461 | 828 . . . 1541 | 2649 |
| BRHIP2009474 | 462 | 38 . . . 469 | 2650 |
| BRHIP2013699 | 463 | 816 . . . 1328 | 2651 |
| BRHIP2014228 | 464 | 444 . . . 869 | 2652 |
| BRHIP2021615 | 465 | 282 . . . 1310 | 2653 |
| BRHIP2022221 | 466 | 2101 . . . 2478 | 2654 |
| BRHIP2024146 | 467 | 177 . . . 539 | 2655 |
| BRHIP2024165 | 468 | 923 . . . 1294 | 2656 |
| BRHIP2026061 | 469 | 282 . . . 1022 | 2657 |
| BRHIP2026288 | 470 | 668 . . . 1681 | 2658 |
| BRHIP2029176 | 471 | 778 . . . 1224 | 2659 |
| BRHIP2029393 | 472 | 2401 . . . 2931 | 2660 |
| BRHIP3000339 | 473 | 2793 . . . 3227 | 2661 |
| BRHIP3000526 | 474 | 107 . . . 2830 | 2662 |
| BRHIP3001283 | 475 | 626 . . . 1645 | 2663 |
| BRHIP3006683 | 476 | 292 . . . 630 | 2664 |
| BRHIP3007483 | 477 | 2669 . . . 3082 | 2665 |
| BRHIP3007586 | 478 | 1409 . . . 2047 | 2666 |
| BRHIP3008183 | 479 | 607 . . . 3861 | 2667 |
| BRHIP3008313 | 480 | 1604 . . . 2353 | 2668 |
| BRHIP3008344 | 481 | 1878 . . . 3719 | 2669 |
| BRHIP3008405 | 482 | 2679 . . . 3668 | 2670 |
| BRHIP3008565 | 483 | 1696 . . . 2055 | 2671 |
| BRHIP3008598 | 484 | 612 . . . 1079 | 2672 |
| BRHIP3008997 | 485 | 1196 . . . 1579 | 2673 |
| BRHIP3009099 | 486 | 74 . . . 892 | 2674 |
| BRHIP3009448 | 487 | 2785 . . . 3354 | 2675 |
| BRHIP3011241 | 488 | 220 . . . 642 | 2676 |
| BRHIP3013765 | 489 | 91 . . . 486 | 2677 |
| BRHIP3013897 | 490 | 2340 . . . 2843 | 2678 |
| BRHIP3015751 | 491 | 2103 . . . 2516 | 2679 |
| BRHIP3016213 | 492 | 402 . . . 1202 | 2680 |
| BRHIP3018797 | 493 | 2687 . . . 3004 | 2681 |
| BRHIP3020182 | 494 | 1515 . . . 2087 | 2682 |
| BRHIP3024118 | 495 | 67 . . . 1131 | 2683 |
| BRHIP3024533 | 496 | 236 . . . >3741 | 2684 |
| BRHIP3024725 | 497 | 1840 . . . 4176 | 2685 |
| BRHIP3025161 | 498 | 225 . . . 4517 | 2686 |
| BRHIP3025702 | 499 | 3307 . . . 3609 | 2687 |
| BRHIP3026097 | 500 | 1387 . . . 1773 | 2688 |
| BRHIP3027137 | 501 | 261 . . . 1673 | 2689 |
| BRHIP3027854 | 502 | 2503 . . . 3213 | 2690 |
| BRSSN2000684 | 503 | 166 . . . >1937 | 2691 |
| BRSSN2003086 | 504 | 1 . . . 342 | 2692 |
| BRSSN2004496 | 505 | 303 . . . 1469 | 2693 |
| BRSSN2004719 | 506 | 62 . . . 784 | 2694 |
| BRSSN2006892 | 507 | 1899 . . . 2396 | 2695 |
| BRSSN2008549 | 508 | 563 . . . 1564 | 2696 |
| BRSSN2008797 | 509 | 57 . . . 1571 | 2697 |
| BRSSN2011262 | 510 | 326 . . . 694 | 2698 |
| BRSSN2011738 | 511 | 1587 . . . 1928 | 2699 |
| BRSSN2013874 | 512 | 1430 . . . 1894 | 2700 |
| BRSSN2014299 | 513 | 641 . . . 1855 | 2701 |
| BRSSN2014424 | 514 | 861 . . . 2201 | 2702 |
| BRSSN2014556 | 515 | 359 . . . 679 | 2703 |
| BRSSN2018581 | 516 | 1440 . . . 1757 | 2704 |
| BRSSN2018925 | 517 | 31 . . . 372 | 2705 |
| BRSTN2000872 | 518 | 537 . . . 1649 | 2706 |
| BRSTN2001067 | 519 | 698 . . . 1048 | 2707 |
| BRSTN2001613 | 520 | 98 . . . 754 | 2708 |
| BRSTN2002400 | 521 | 82 . . . 1170 | 2709 |
| BRSTN2003835 | 522 | 25 . . . 420 | 2710 |
| BRSTN2004863 | 523 | 438 . . . 2264 | 2711 |
| BRSTN2004987 | 524 | 11 . . . 1093 | 2712 |
| BRSTN2005721 | 525 | 415 . . . 1302 | 2713 |
| BRSTN2006865 | 526 | 693 . . . 1559 | 2714 |
| BRSTN2007000 | 527 | 115 . . . 492 | 2715 |
| BRSTN2007284 | 528 | 96 . . . 608 | 2716 |
| BRSTN2008052 | 529 | 133 . . . 483 | 2717 |
| BRSTN2008283 | 530 | 179 . . . 484 | 2718 |
| BRSTN2008418 | 531 | 468 . . . 1013 | 2719 |
| BRSTN2008457 | 532 | 942 . . . 1331 | 2720 |
| BRSTN2009899 | 533 | 707 . . . 1135 | 2721 |
| BRSTN2010363 | 534 | 1125 . . . 1901 | 2722 |
| BRSTN2010500 | 535 | 703 . . . 1134 | 2723 |
| BRSTN2010750 | 536 | 247 . . . 1290 | 2724 |
| BRSTN2012320 | 537 | 300 . . . 686 | 2725 |
| BRSTN2012380 | 538 | 1068 . . . 1439 | 2726 |
| BRSTN2013741 | 539 | 648 . . . 1274 | 2727 |
| BRSTN2015015 | 540 | 1290 . . . 1610 | 2728 |
| BRSTN2016470 | 541 | 479 . . . 814 | 2729 |
| BRSTN2016678 | 542 | 97 . . . 486 | 2730 |
| BRSTN2017084 | 543 | 53 . . . 397 | 2731 |
| BRSTN2017110 | 544 | 128 . . . 562 | 2732 |
| BRSTN2017237 | 545 | 42 . . . 1466 | 2733 |
| BRSTN2017771 | 546 | 1018 . . . 1332 | 2734 |
| BRSTN2018083 | 547 | 309 . . . 641 | 2735 |
| BRSTN2019129 | 548 | 1004 . . . 1459 | 2736 |
| BRTHA1000311 | 549 | 210 . . . 680 | 2737 |
| BRTHA2000855 | 550 | 149 . . . >2176 | 2738 |
| BRTHA2001462 | 551 | 710 . . . 1555 | 2739 |
| BRTHA2002115 | 552 | 947 . . . 1267 | 2740 |
| BRTHA2002281 | 553 | 856 . . . 1767 | 2741 |
| BRTHA2002376 | 554 | 124 . . . 513 | 2742 |
| BRTHA2002442 | 555 | 28 . . . 630 | 2743 |
| BRTHA2002493 | 556 | 400 . . . 735 | 2744 |
| BRTHA2002608 | 557 | 1100 . . . 1495 | 2745 |
| BRTHA2002808 | 558 | 1010 . . . 1456 | 2746 |
| BRTHA2003030 | 559 | 501 . . . 875 | 2747 |
| BRTHA2003110 | 560 | 1429 . . . 2010 | 2748 |
| BRTHA2003116 | 561 | 78 . . . 476 | 2749 |
| BRTHA2003461 | 562 | 2179 . . . 2709 | 2750 |
| BRTHA2004821 | 563 | 1764 . . . 2273 | 2751 |
| BRTHA2004978 | 564 | 125 . . . 937 | 2752 |
| BRTHA2005579 | 565 | 44 . . . 2488 | 2753 |
| BRTHA2005956 | 566 | 1466 . . . 1882 | 2754 |
| BRTHA2006075 | 567 | 591 . . . 995 | 2755 |
| BRTHA2006146 | 568 | 24 . . . 569 | 2756 |
| BRTHA2006194 | 569 | 129 . . . 506 | 2757 |
| BRTHA2007122 | 570 | 297 . . . 2267 | 2758 |
| BRTHA2007422 | 571 | 114 . . . 632 | 2759 |
| BRTHA2007603 | 572 | 534 . . . 962 | 2760 |
| BRTHA2008316 | 573 | 124 . . . 429 | 2761 |
| BRTHA2008335 | 574 | 1205 . . . 1747 | 2762 |
| BRTHA2008527 | 575 | 54 . . . 680 | 2763 |
| BRTHA2008535 | 576 | 2076 . . . 2597 | 2764 |
| BRTHA2008955 | 577 | 427 . . . 1395 | 2765 |
| BRTHA2009311 | 578 | 47 . . . 502 | 2766 |
| BRTHA2009846 | 579 | 492 . . . 881 | 2767 |
| BRTHA2009972 | 580 | 134 . . . 493 | 2768 |
| BRTHA2010073 | 581 | 1064 . . . 1399 | 2769 |
| BRTHA2010608 | 582 | 926 . . . 1354 | 2770 |
| BRTHA2010884 | 583 | 76 . . . 1305 | 2771 |
| BRTHA2010907 | 584 | 78 . . . 806 | 2772 |
| BRTHA2011194 | 585 | 2370 . . . 2720 | 2773 |
| BRTHA2011351 | 586 | 18 . . . 905 | 2774 |
| BRTHA2011500 | 587 | 123 . . . 476 | 2775 |
| BRTHA2011641 | 588 | 47 . . . 562 | 2776 |
| BRTHA2012392 | 589 | 1425 . . . 2021 | 2777 |
| BRTHA2012562 | 590 | 1649 . . . 1963 | 2778 |
| BRTHA2012980 | 591 | 610 . . . 1290 | 2779 |
| BRTHA2013262 | 592 | 138 . . . 794 | 2780 |
| BRTHA2013460 | 593 | 379 . . . 732 | 2781 |
| BRTHA2013707 | 594 | 244 . . . 657 | 2782 |
| BRTHA2014792 | 595 | 221 . . . 1663 | 2783 |
| BRTHA2014828 | 596 | 667 . . . 1158 | 2784 |
| BRTHA2015406 | 597 | 162 . . . 1583 | 2785 |
| BRTHA2015478 | 598 | 111 . . . 419 | 2786 |
| BRTHA2015696 | 599 | 1109 . . . 1657 | 2787 |
| BRTHA2015878 | 600 | 433 . . . 1902 | 2788 |
| BRTHA2016215 | 601 | 1831 . . . 2280 | 2789 |
| BRTHA2016496 | 602 | 76 . . . 1527 | 2790 |
| BRTHA2016543 | 603 | 310 . . . 636 | 2791 |
| BRTHA2017353 | 604 | 68 . . . 1027 | 2792 |
| BRTHA2017985 | 605 | 166 . . . 474 | 2793 |
| BRTHA2018165 | 606 | 1224 . . . 1550 | 2794 |
| BRTHA2018344 | 607 | 1064 . . . 1402 | 2795 |
| BRTHA2018591 | 608 | 515 . . . 1195 | 2796 |
| BRTHA2018624 | 609 | 1253 . . . 2293 | 2797 |
| BRTHA2018707 | 610 | 1333 . . . 1791 | 2798 |
| BRTHA2019014 | 611 | 134 . . . 451 | 2799 |
| BRTHA2019022 | 612 | 189 . . . 494 | 2800 |
| BRTHA2019048 | 613 | 52 . . . 549 | 2801 |
| BRTHA3000273 | 614 | 128 . . . 694 | 2802 |
| BRTHA3000297 | 615 | 1553 . . . 2083 | 2803 |
| BRTHA3000633 | 616 | 40 . . . 858 | 2804 |
| BRTHA3001721 | 617 | 382 . . . 963 | 2805 |
| BRTHA3002401 | 618 | 944 . . . 1396 | 2806 |
| BRTHA3002427 | 619 | 871 . . . 2589 | 2807 |
| BRTHA3002933 | 620 | 835 . . . 1908 | 2808 |
| BRTHA3003074 | 621 | 545 . . . 2050 | 2809 |
| BRTHA3003343 | 622 | 1938 . . . 2639 | 2810 |
| BRTHA3003449 | 623 | 1281 . . . 2612 | 2811 |
| BRTHA3003474 | 624 | 6 . . . 410 | 2812 |
| BRTHA3003490 | 625 | 22 . . . 3783 | 2813 |
| BRTHA3004475 | 626 | 1777 . . . 2196 | 2814 |
| BRTHA3005046 | 627 | 14 . . . 415 | 2815 |
| BRTHA3006856 | 628 | 241 . . . 624 | 2816 |
| BRTHA3007113 | 629 | 656 . . . 1657 | 2817 |
| BRTHA3007148 | 630 | 70 . . . 960 | 2818 |
| BRTHA3007319 | 631 | 1348 . . . 1995 | 2819 |
| BRTHA3007769 | 632 | 17 . . . 4579 | 2820 |
| BRTHA3008143 | 633 | 1649 . . . 1987 | 2821 |
| BRTHA3008310 | 634 | 1333 . . . 2673 | 2822 |
| BRTHA3008386 | 635 | 599 . . . 1921 | 2823 |
| BRTHA3008520 | 636 | 1670 . . . 2659 | 2824 |
| BRTHA3008778 | 637 | 1533 . . . 3353 | 2825 |
| BRTHA3009037 | 638 | 175 . . . 3441 | 2826 |
| BRTHA3009090 | 639 | 188 . . . 4141 | 2827 |
| BRTHA3009291 | 640 | 174 . . . 782 | 2828 |
| BRTHA3010366 | 641 | 1998 . . . 3029 | 2829 |
| BRTHA3013884 | 642 | 1085 . . . 3331 | 2830 |
| BRTHA3015815 | 643 | 1172 . . . 2122 | 2831 |
| BRTHA3015910 | 644 | 944 . . . 2899 | 2832 |
| BRTHA3016845 | 645 | 403 . . . 771 | 2833 |
| BRTHA3016917 | 646 | 1674 . . . 3179 | 2834 |
| BRTHA3017047 | 647 | 146 . . . 1060 | 2835 |
| BRTHA3017589 | 648 | 14 . . . 793 | 2836 |
| BRTHA3017848 | 649 | 218 . . . 1342 | 2837 |
| BRTHA3018514 | 650 | 416 . . . 1567 | 2838 |
| BRTHA3018617 | 651 | 28 . . . 522 | 2839 |
| BRTHA3018656 | 652 | 247 . . . 1338 | 2840 |
| BRTHA3019105 | 653 | 266 . . . 682 | 2841 |
| CERVX1000042 | 654 | 3 . . . 536 | 2842 |
| CERVX2002006 | 655 | 874 . . . 1257 | 2843 |
| COLON1000030 | 656 | 607 . . . 1170 | 2844 |
| COLON2000470 | 657 | 9 . . . 656 | 2845 |
| COLON2000568 | 658 | 73 . . . 1506 | 2846 |
| COLON2001721 | 659 | 1274 . . . 1981 | 2847 |
| COLON2002443 | 660 | 13 . . . 447 | 2848 |
| COLON2002520 | 661 | 1243 . . . >3130 | 2849 |
| COLON2003043 | 662 | 3 . . . 311 | 2850 |
| COLON2004478 | 663 | 81 . . . 1565 | 2851 |
| COLON2005126 | 664 | 244 . . . 747 | 2852 |
| COLON2005772 | 665 | 286 . . . 816 | 2853 |
| COLON2006282 | 666 | 200 . . . 505 | 2854 |
| COLON2009499 | 667 | 13 . . . 345 | 2855 |
| CORDB1000140 | 668 | 346 . . . 897 | 2856 |
| CORDB2000061 | 669 | 12 . . . 329 | 2857 |
| CORDB2000541 | 670 | 495 . . . 1277 | 2858 |
| CTONG1000087 | 671 | 194 . . . 2326 | 2859 |
| CTONG1000088 | 672 | 2902 . . . 3252 | 2860 |
| CTONG1000288 | 673 | 1315 . . . 1875 | 2861 |
| CTONG1000302 | 674 | 168 . . . 485 | 2862 |
| CTONG1000341 | 675 | 29 . . . 1702 | 2863 |
| CTONG1000467 | 676 | 90 . . . 2312 | 2864 |
| CTONG1000488 | 677 | 2227 . . . 2892 | 2865 |
| CTONG1000508 | 678 | 1182 . . . 2615 | 2866 |
| CTONG1000540 | 679 | 778 . . . 1428 | 2867 |
| CTONG2000042 | 680 | 75 . . . 2603 | 2868 |
| CTONG2001877 | 681 | 11 . . . 721 | 2869 |
| CTONG2004062 | 682 | 121 . . . 2268 | 2870 |
| CTONG2006798 | 683 | 423 . . . >2466 | 2871 |
| CTONG2008233 | 684 | 655 . . . 1893 | 2872 |
| CTONG2009423 | 685 | 1433 . . . 1774 | 2873 |
| CTONG2009531 | 686 | 49 . . . 543 | 2874 |
| CTONG2010803 | 687 | 1352 . . . 2464 | 2875 |
| CTONG2013178 | 688 | 50 . . . 1366 | 2876 |
| CTONG2017500 | 689 | 57 . . . 878 | 2877 |
| CTONG2019248 | 690 | 3528 . . . 3938 | 2878 |
| CTONG2019652 | 691 | 229 . . . 540 | 2879 |
| CTONG2019704 | 692 | 176 . . . 799 | 2880 |
| CTONG2019788 | 693 | 1985 . . . 2311 | 2881 |
| CTONG2019833 | 694 | 1026 . . . 1328 | 2882 |
| CTONG2020026 | 695 | 152 . . . 2845 | 2883 |
| CTONG2020127 | 696 | 1343 . . . 1774 | 2884 |
| CTONG2020522 | 697 | 191 . . . 586 | 2885 |
| CTONG2020638 | 698 | 159 . . . 986 | 2886 |
| CTONG2020806 | 699 | 436 . . . 933 | 2887 |
| CTONG2021132 | 700 | 1883 . . . 2233 | 2888 |
| CTONG2022153 | 701 | 341 . . . 787 | 2889 |
| CTONG2022601 | 702 | 1301 . . . 1621 | 2890 |
| CTONG2023021 | 703 | 2277 . . . 2840 | 2891 |
| CTONG2023512 | 704 | 311 . . . 1624 | 2892 |
| CTONG2024206 | 705 | 322 . . . 2499 | 2893 |
| CTONG2024749 | 706 | 129 . . . 2198 | 2894 |
| CTONG2025496 | 707 | 151 . . . 3015 | 2895 |
| CTONG2025516 | 708 | 459 . . . 773 | 2896 |
| CTONG2025900 | 709 | 135 . . . 2399 | 2897 |
| CTONG2026920 | 710 | 363 . . . 689 | 2898 |
| CTONG2027327 | 711 | 396 . . . 719 | 2899 |
| CTONG2028124 | 712 | 761 . . . 2683 | 2900 |
| CTONG2028687 | 713 | 654 . . . 1967 | 2901 |
| CTONG3000084 | 714 | 1 . . . 3111 | 2902 |
| CTONG3000657 | 715 | 31 . . . 732 | 2903 |
| CTONG3000686 | 716 | 2605 . . . 3294 | 2904 |
| CTONG3000707 | 717 | 2912 . . . 3265 | 2905 |
| CTONG3000896 | 718 | 3245 . . . 3676 | 2906 |
| CTONG3001123 | 719 | 285 . . . 3392 | 2907 |
| CTONG3001370 | 720 | 26 . . . 3922 | 2908 |
| CTONG3001420 | 721 | 536 . . . 850 | 2909 |
| CTONG3001560 | 722 | 19 . . . 450 | 2910 |
| CTONG3002020 | 723 | 242 . . . 673 | 2911 |
| CTONG3002127 | 724 | 1264 . . . 2604 | 2912 |
| CTONG3002412 | 725 | 1782 . . . >3812 | 2913 |
| CTONG3002674 | 726 | 258 . . . >4088 | 2914 |
| CTONG3003179 | 727 | 2650 . . . 3144 | 2915 |
| CTONG3003483 | 728 | 3006 . . . 3332 | 2916 |
| CTONG3003652 | 729 | 229 . . . 3372 | 2917 |
| CTONG3003654 | 730 | 1865 . . . 2263 | 2918 |
| CTONG3003737 | 731 | 87 . . . 1832 | 2919 |
| CTONG3003905 | 732 | 75 . . . 3833 | 2920 |
| CTONG3003972 | 733 | 203 . . . 2167 | 2921 |
| CTONG3004072 | 734 | 110 . . . 3784 | 2922 |
| CTONG3004712 | 735 | 3159 . . . 3890 | 2923 |
| CTONG3005325 | 736 | 210 . . . 3044 | 2924 |
| CTONG3005648 | 737 | 180 . . . 1061 | 2925 |
| CTONG3005713 | 738 | 42 . . . 902 | 2926 |
| CTONG3005813 | 739 | 173 . . . 991 | 2927 |
| CTONG3006067 | 740 | 194 . . . 583 | 2928 |
| CTONG3006186 | 741 | 208 . . . 1869 | 2929 |
| CTONG3006650 | 742 | 299 . . . 649 | 2930 |
| CTONG3007444 | 743 | 1638 . . . 2039 | 2931 |
| CTONG3007528 | 744 | 177 . . . 962 | 2932 |
| CTONG3007586 | 745 | 27 . . . 3089 | 2933 |
| CTONG3007870 | 746 | 1402 . . . 2064 | 2934 |
| CTONG3008252 | 747 | 216 . . . 572 | 2935 |
| CTONG3008258 | 748 | 194 . . . 2320 | 2936 |
| CTONG3008496 | 749 | 1412 . . . 1774 | 2937 |
| CTONG3008566 | 750 | 2577 . . . 3113 | 2938 |
| CTONG3008639 | 751 | 102 . . . >4092 | 2939 |
| CTONG3008831 | 752 | 198 . . . 3077 | 2940 |
| CTONG3008894 | 753 | 1230 . . . 2411 | 2941 |
| CTONG3008951 | 754 | 3010 . . . 3438 | 2942 |
| CTONG3009028 | 755 | 25 . . . 3954 | 2943 |
| CTONG3009227 | 756 | 1948 . . . 2337 | 2944 |
| CTONG3009239 | 757 | 2907 . . . 3275 | 2945 |
| CTONG3009328 | 758 | 101 . . . 1291 | 2946 |
| CTONG3009385 | 759 | 795 . . . 2390 | 2947 |
| D30ST2002182 | 760 | 79 . . . 1308 | 2948 |
| D30ST2002648 | 761 | 215 . . . 1225 | 2949 |
| D30ST3000169 | 762 | 83 . . . 1204 | 2950 |
| DFNES1000107 | 763 | 651 . . . 986 | 2951 |
| DFNES2000146 | 764 | 56 . . . 520 | 2952 |
| DFNES2001108 | 765 | 435 . . . 854 | 2953 |
| DFNES2005266 | 766 | 285 . . . 926 | 2954 |
| DFNES2010502 | 767 | 108 . . . 479 | 2955 |
| DFNES2011239 | 768 | 80 . . . 1081 | 2956 |
| DFNES2011499 | 769 | 206 . . . 595 | 2957 |
| ERLTF2000324 | 770 | 66 . . . 641 | 2958 |
| FCBBF1000297 | 771 | 1409 . . . >2811 | 2959 |
| FCBBF2001183 | 772 | 951 . . . 1559 | 2960 |
| FCBBF2007510 | 773 | 474 . . . 1082 | 2961 |
| FCBBF3001977 | 774 | 1167 . . . 1535 | 2962 |
| FCBBF3002163 | 775 | 86 . . . 2905 | 2963 |
| FCBBF3003435 | 776 | 618 . . . 926 | 2964 |
| FCBBF3004502 | 777 | 145 . . . 2565 | 2965 |
| FCBBF3004847 | 778 | 378 . . . 830 | 2966 |
| FCBBF3006171 | 779 | 599 . . . 913 | 2967 |
| FCBBF3007242 | 780 | 93 . . . 416 | 2968 |
| FCBBF3007540 | 781 | 307 . . . 1269 | 2969 |
| FCBBF3008944 | 782 | 772 . . . 1377 | 2970 |
| FCBBF3009888 | 783 | 103 . . . 597 | 2971 |
| FCBBF3012170 | 784 | 89 . . . 1054 | 2972 |
| FCBBF3012288 | 785 | 1881 . . . 2906 | 2973 |
| FCBBF3013307 | 786 | 300 . . . >2766 | 2974 |
| FCBBF3013846 | 787 | 2050 . . . 2673 | 2975 |
| FCBBF3021576 | 788 | 304 . . . 678 | 2976 |
| FCBBF3021940 | 789 | 353 . . . 772 | 2977 |
| FCBBF3023443 | 790 | 1109 . . . 1495 | 2978 |
| FCBBF3023895 | 791 | 702 . . . 1409 | 2979 |
| FCBBF3025730 | 792 | 333 . . . 941 | 2980 |
| FCBBF3027717 | 793 | 218 . . . >4477 | 2981 |
| FCBBF4000076 | 794 | 225 . . . 566 | 2982 |
| FEBRA1000030 | 795 | 735 . . . 1358 | 2983 |
| FEBRA2000253 | 796 | 1692 . . . 2822 | 2984 |
| FEBRA2006396 | 797 | 442 . . . 1521 | 2985 |
| FEBRA2007544 | 798 | 321 . . . 1874 | 2986 |
| FEBRA2007708 | 799 | 356 . . . 1981 | 2987 |
| FEBRA2007793 | 800 | 407 . . . 973 | 2988 |
| FEBRA2007801 | 801 | 362 . . . 2185 | 2989 |
| FEBRA2008287 | 802 | 1110 . . . 2531 | 2990 |
| FEBRA2008311 | 803 | 80 . . . 1339 | 2991 |
| FEBRA2008360 | 804 | 428 . . . 997 | 2992 |
| FEBRA2008468 | 805 | 291 . . . 1496 | 2993 |
| FEBRA2010719 | 806 | 348 . . . 995 | 2994 |
| FEBRA2014213 | 807 | 407 . . . 1483 | 2995 |
| FEBRA2015588 | 808 | 1131 . . . 1916 | 2996 |
| FEBRA2020484 | 809 | 1160 . . . 1615 | 2997 |
| FEBRA2020582 | 810 | 289 . . . 729 | 2998 |
| FEBRA2020668 | 811 | 787 . . . 1839 | 2999 |
| FEBRA2020886 | 812 | 1749 . . . 2342 | 3000 |
| FEBRA2021339 | 813 | 441 . . . 914 | 3001 |
| FEBRA2021571 | 814 | 566 . . . 1591 | 3002 |
| FEBRA2021908 | 815 | 181 . . . 588 | 3003 |
| FEBRA2021966 | 816 | 102 . . . 476 | 3004 |
| FEBRA2024136 | 817 | 2261 . . . 2638 | 3005 |
| FEBRA2024150 | 818 | 15 . . . 950 | 3006 |
| FEBRA2024343 | 819 | 2284 . . . 3036 | 3007 |
| FEBRA2024744 | 820 | 918 . . . 1781 | 3008 |
| FEBRA2025427 | 821 | 201 . . . 548 | 3009 |
| FEBRA2026984 | 822 | 910 . . . 2496 | 3010 |
| FEBRA2027082 | 823 | 174 . . . 509 | 3011 |
| FEBRA2027297 | 824 | 1040 . . . 1396 | 3012 |
| FEBRA2027352 | 825 | 604 . . . 1065 | 3013 |
| FEBRA2028366 | 826 | 5 . . . 994 | 3014 |
| FEBRA2028477 | 827 | 2176 . . . 2685 | 3015 |
| FEBRA2028618 | 828 | 587 . . . 1054 | 3016 |
| HCASM2001301 | 829 | 628 . . . 1050 | 3017 |
| HCASM2002502 | 830 | 112 . . . 942 | 3018 |
| HCASM2002918 | 831 | 292 . . . >1851 | 3019 |
| HCASM2003212 | 832 | 304 . . . 6 | 3020 |
| HCASM2003415 | 833 | 23 . . . 3 | 3021 |
| HCASM2007047 | 834 | 79 . . . 2250 | 3022 |
| HCASM2007737 | 835 | 54 . . . 548 | 3023 |
| HCHON2000028 | 836 | 32 . . . 2239 | 3024 |
| HCHON2000212 | 837 | 1713 . . . 2087 | 3025 |
| HCHON2000244 | 838 | 698 . . . 1528 | 3026 |
| HCHON2000418 | 839 | 1185 . . . 1487 | 3027 |
| HCHON2000626 | 840 | 1168 . . . 1578 | 3028 |
| HCHON2001084 | 841 | 164 . . . 2017 | 3029 |
| HCHON2001217 | 842 | 169 . . . 2856 | 3030 |
| HCHON2001548 | 843 | 1460 . . . 1942 | 3031 |
| HCHON2001577 | 844 | 14 . . . 1726 | 3032 |
| HCHON2001712 | 845 | 143 . . . 1624 | 3033 |
| HCHON2002676 | 846 | 42 . . . 2069 | 3034 |
| HCHON2003532 | 847 | 614 . . . 1618 | 3035 |
| HCHON2004007 | 848 | 181 . . . 1917 | 3036 |
| HCHON2004531 | 849 | 299 . . . 1528 | 3037 |
| HCHON2004776 | 850 | 158 . . . 1723 | 3038 |
| HCHON2005921 | 851 | 174 . . . 860 | 3039 |
| HCHON2006250 | 852 | 517 . . . 2151 | 3040 |
| HCHON2006714 | 853 | 51 . . . 371 | 3041 |
| HCHON2007881 | 854 | 77 . . . 1801 | 3042 |
| HCHON2008112 | 855 | 929 . . . 1462 | 3043 |
| HCHON2008444 | 856 | 1810 . . . 2112 | 3044 |
| HEART1000010 | 857 | 967 . . . 1296 | 3045 |
| HEART1000074 | 858 | 151 . . . 1626 | 3046 |
| HEART1000088 | 859 | 1567 . . . 2016 | 3047 |
| HEART1000139 | 860 | 1408 . . . 2106 | 3048 |
| HEART2001680 | 861 | 59 . . . 1549 | 3049 |
| HEART2001756 | 862 | 155 . . . 1795 | 3050 |
| HEART2006131 | 863 | 8 . . . 1717 | 3051 |
| HEART2006909 | 864 | 4 . . . 927 | 3052 |
| HEART2007031 | 865 | 106 . . . 1374 | 3053 |
| HEART2010391 | 866 | 619 . . . 1173 | 3054 |
| HEART2010492 | 867 | 91 . . . 1617 | 3055 |
| HEART2010495 | 868 | 148 . . . >2295 | 3056 |
| HHDPC1000118 | 869 | 258 . . . 2489 | 3057 |
| HHDPC2001337 | 870 | 210 . . . 566 | 3058 |
| HLUNG1000017 | 871 | 227 . . . 1054 | 3059 |
| HLUNG2000014 | 872 | 175 . . . 816 | 3060 |
| HLUNG2001996 | 873 | 990 . . . 1562 | 3061 |
| HLUNG2002465 | 874 | 72 . . . 1796 | 3062 |
| HLUNG2002958 | 875 | 123 . . . 2234 | 3063 |
| HLUNG2003003 | 876 | 459 . . . 848 | 3064 |
| HLUNG2003872 | 877 | 542 . . . 1366 | 3065 |
| HLUNG2010464 | 878 | 256 . . . 972 | 3066 |
| HLUNG2011041 | 879 | 721 . . . 1422 | 3067 |
| HLUNG2011298 | 880 | 1669 . . . 1983 | 3068 |
| HLUNG2012049 | 881 | 264 . . . 755 | 3069 |
| HLUNG2012287 | 882 | 789 . . . 1139 | 3070 |
| HLUNG2012727 | 883 | 198 . . . 1046 | 3071 |
| HLUNG2013204 | 884 | 1196 . . . 1513 | 3072 |
| HLUNG2013304 | 885 | 329 . . . 649 | 3073 |
| HLUNG2013622 | 886 | 123 . . . 440 | 3074 |
| HLUNG2013851 | 887 | 224 . . . 787 | 3075 |
| HLUNG2014262 | 888 | 189 . . . 1886 | 3076 |
| HLUNG2014288 | 889 | 192 . . . 1106 | 3077 |
| HLUNG2014449 | 890 | 2235 . . . >2713 | 3078 |
| HLUNG2015617 | 891 | 1630 . . . 1968 | 3079 |
| HLUNG2017350 | 892 | 879 . . . 1766 | 3080 |
| HLUNG2017546 | 893 | 1666 . . . 2070 | 3081 |
| HLUNG2017806 | 894 | 29 . . . 577 | 3082 |
| HLUNG2019058 | 895 | 239 . . . 568 | 3083 |
| HSYRA2004858 | 896 | 150 . . . 467 | 3084 |
| HSYRA2005456 | 897 | 61 . . . 2415 | 3085 |
| HSYRA2005496 | 898 | 36 . . . 1211 | 3086 |
| HSYRA2006873 | 899 | 590 . . . 910 | 3087 |
| HSYRA2007667 | 900 | 424 . . . 1281 | 3088 |
| HSYRA2008376 | 901 | 329 . . . 2422 | 3089 |
| HSYRA2008714 | 902 | 249 . . . 1487 | 3090 |
| HSYRA2009075 | 903 | 237 . . . 2204 | 3091 |
| HSYRA2009102 | 904 | 119 . . . 1357 | 3092 |
| IMR322000127 | 905 | 847 . . . 2271 | 3093 |
| IMR322000917 | 906 | 287 . . . 1000 | 3094 |
| IMR322001380 | 907 | 14 . . . 790 | 3095 |
| IMR322002035 | 908 | 2228 . . . 2620 | 3096 |
| IMR322002110 | 909 | 176 . . . 553 | 3097 |
| IMR322003675 | 910 | 202 . . . 978 | 3098 |
| IMR322006222 | 911 | 516 . . . 845 | 3099 |
| IMR322006495 | 912 | 63 . . . 749 | 3100 |
| IMR322006886 | 913 | 565 . . . 1224 | 3101 |
| IMR322007225 | 914 | 24 . . . 467 | 3102 |
| IMR322016146 | 915 | 112 . . . 426 | 3103 |
| IMR322018117 | 916 | 37 . . . 528 | 3104 |
| KIDNE1000064 | 917 | 302 . . . 1963 | 3105 |
| KIDNE2000665 | 918 | 379 . . . 735 | 3106 |
| KIDNE2000722 | 919 | 227 . . . 571 | 3107 |
| KIDNE2000832 | 920 | 103 . . . 1554 | 3108 |
| KIDNE2000846 | 921 | 23 . . . 700 | 3109 |
| KIDNE2001361 | 922 | 953 . . . 2056 | 3110 |
| KIDNE2001847 | 923 | 86 . . . 1240 | 3111 |
| KIDNE2002252 | 924 | 96 . . . 2543 | 3112 |
| KIDNE2002991 | 925 | 644 . . . 952 | 3113 |
| KIDNE2003837 | 926 | 1732 . . . 2073 | 3114 |
| KIDNE2005543 | 927 | 2011 . . . 2484 | 3115 |
| KIDNE2006580 | 928 | 40 . . . 1617 | 3116 |
| KIDNE2010264 | 929 | 640 . . . 1158 | 3117 |
| KIDNE2011314 | 930 | 796 . . . 1098 | 3118 |
| KIDNE2011532 | 931 | 1097 . . . 1636 | 3119 |
| KIDNE2011635 | 932 | 148 . . . 2376 | 3120 |
| KIDNE2012945 | 933 | 301 . . . 1407 | 3121 |
| KIDNE2013095 | 934 | 162 . . . 749 | 3122 |
| LIVER2007415 | 935 | 308 . . . 1144 | 3123 |
| LYMPB1000141 | 936 | 85 . . . 396 | 3124 |
| LYMPB2000083 | 937 | 35 . . . 1084 | 3125 |
| MESAN2001979 | 938 | 1199 . . . 1855 | 3126 |
| MESAN2006563 | 939 | 264 . . . 1670 | 3127 |
| MESAN2012054 | 940 | 24 . . . 1892 | 3128 |
| MESAN2014295 | 941 | 1349 . . . 1687 | 3129 |
| MESAN2015515 | 942 | 381 . . . 803 | 3130 |
| MESAN2018576 | 943 | 65 . . . 370 | 3131 |
| MESTC1000042 | 944 | 235 . . . 738 | 3132 |
| MESTC2000153 | 945 | 350 . . . 820 | 3133 |
| NB9N41000340 | 946 | 625 . . . 1032 | 3134 |
| NCRRP1000129 | 947 | 147 . . . 521 | 3135 |
| NESOP2000744 | 948 | 652 . . . 1020 | 3136 |
| NESOP2001433 | 949 | 81 . . . 1580 | 3137 |
| NESOP2001656 | 950 | 369 . . . 707 | 3138 |
| NESOP2001694 | 951 | 240 . . . 731 | 3139 |
| NESOP2001752 | 952 | 624 . . . 935 | 3140 |
| NESOP2002738 | 953 | 1342 . . . 1890 | 3141 |
| NHNPC2000606 | 954 | 1635 . . . 1949 | 3142 |
| NHNPC2000877 | 955 | 27 . . . 443 | 3143 |
| NHNPC2001223 | 956 | 2476 . . . 2790 | 3144 |
| NHNPC2001816 | 957 | 1053 . . . 1421 | 3145 |
| NHNPC2002565 | 958 | 463 . . . 1134 | 3146 |
| NHNPC2002749 | 959 | 81 . . . 605 | 3147 |
| NOVAR2000136 | 960 | 174 . . . 1481 | 3148 |
| NOVAR2000710 | 961 | 59 . . . 466 | 3149 |
| NOVAR2000962 | 962 | 25 . . . 393 | 3150 |
| NOVAR2001108 | 963 | 49 . . . 1482 | 3151 |
| NOVAR2001783 | 964 | 44 . . . 490 | 3152 |
| NT2NE2003252 | 965 | 889 . . . 1899 | 3153 |
| NT2NE2005890 | 966 | 435 . . . 740 | 3154 |
| NT2NE2006531 | 967 | 295 . . . 1812 | 3155 |
| NT2NE2006909 | 968 | 661 . . . 1647 | 3156 |
| NT2NE2008060 | 969 | 1027 . . . 1404 | 3157 |
| NT2RI2003993 | 970 | 600 . . . 1136 | 3158 |
| NT2RI2004618 | 971 | 495 . . . 1253 | 3159 |
| NT2RI2005166 | 972 | 60 . . . 1436 | 3160 |
| NT2RI2006686 | 973 | 14 . . . 1909 | 3161 |
| NT2RI2008724 | 974 | 289 . . . 1917 | 3162 |
| NT2RI2009855 | 975 | 1615 . . . 2283 | 3163 |
| NT2RI2011422 | 976 | 110 . . . 1543 | 3164 |
| NT2RI2011683 | 977 | 206 . . . 754 | 3165 |
| NT2RI2012659 | 978 | 100 . . . 528 | 3166 |
| NT2RI2012990 | 979 | 85 . . . 561 | 3167 |
| NT2RI2013357 | 980 | 287 . . . 877 | 3168 |
| NT2RI2014247 | 981 | 1608 . . . 1967 | 3169 |
| NT2RI2014551 | 982 | 52 . . . >2076 | 3170 |
| NT2RI2014733 | 983 | 638 . . . 1204 | 3171 |
| NT2RI2016128 | 984 | 173 . . . 589 | 3172 |
| NT2RI2018311 | 985 | 680 . . . 1345 | 3173 |
| NT2RI2018883 | 986 | 316 . . . 846 | 3174 |
| NT2RI2019751 | 987 | 357 . . . 743 | 3175 |
| NT2RI2023303 | 988 | 1041 . . . 1451 | 3176 |
| NT2RI2025909 | 989 | 208 . . . 1119 | 3177 |
| NT2RI2025957 | 990 | 348 . . . 2432 | 3178 |
| NT2RI2027081 | 991 | 1254 . . . 1586 | 3179 |
| NT2RI2027396 | 992 | 109 . . . 426 | 3180 |
| NT2RI3000622 | 993 | 305 . . . 964 | 3181 |
| NT2RI3001263 | 994 | 2825 . . . 3241 | 3182 |
| NT2RI3001515 | 995 | 857 . . . 3373 | 3183 |
| NT2RI3002303 | 996 | 2269 . . . 2613 | 3184 |
| NT2RI3002842 | 997 | 50 . . . 934 | 3185 |
| NT2RI3002892 | 998 | 3 . . . 311 | 3186 |
| NT2RI3003031 | 999 | 111 . . . 413 | 3187 |
| NT2RI3003095 | 1000 | 1032 . . . 1412 | 3188 |
| NT2RI3003162 | 1001 | 865 . . . 1353 | 3189 |
| NT2RI3003382 | 1002 | 600 . . . 2642 | 3190 |
| NT2RI3003409 | 1003 | 2744 . . . 3340 | 3191 |
| NT2RI3004381 | 1004 | 2637 . . . 3314 | 3192 |
| NT2RI3004510 | 1005 | 178 . . . 1950 | 3193 |
| NT2RI3005202 | 1006 | 132 . . . 659 | 3194 |
| NT2RI3005403 | 1007 | 366 . . . 1073 | 3195 |
| NT2RI3005724 | 1008 | 194 . . . 637 | 3196 |
| NT2RI3006132 | 1009 | 82 . . . 684 | 3197 |
| NT2RI3006171 | 1010 | 300 . . . 1211 | 3198 |
| NT2RI3006284 | 1011 | 154 . . . 1911 | 3199 |
| NT2RI3006340 | 1012 | 335 . . . 5392 | 3200 |
| NT2RI3006376 | 1013 | 152 . . . 4081 | 3201 |
| NT2RI3006673 | 1014 | 197 . . . 3997 | 3202 |
| NT2RI3006796 | 1015 | 1047 . . . 1427 | 3203 |
| NT2RI3007065 | 1016 | 1707 . . . 2096 | 3204 |
| NT2RI3007158 | 1017 | 656 . . . >4499 | 3205 |
| NT2RI3007291 | 1018 | 10 . . . 1614 | 3206 |
| NT2RI3007543 | 1019 | 54 . . . 4181 | 3207 |
| NT2RI3007757 | 1020 | 225 . . . >5460 | 3208 |
| NT2RI3007978 | 1021 | 745 . . . 2505 | 3209 |
| NT2RI3008055 | 1022 | 2452 . . . 2838 | 3210 |
| NT2RI3008162 | 1023 | 81 . . . 2354 | 3211 |
| NT2RI3008652 | 1024 | 179 . . . 2323 | 3212 |
| NT2RI3008697 | 1025 | 334 . . . 1515 | 3213 |
| NT2RI3008974 | 1026 | 949 . . . 1434 | 3214 |
| NT2RI3009158 | 1027 | 228 . . . 1286 | 3215 |
| NT2RP7000359 | 1028 | 48 . . . 3902 | 3216 |
| NT2RP7000466 | 1029 | 72 . . . 2987 | 3217 |
| NT2RP7004027 | 1030 | 507 . . . 1787 | 3218 |
| NT2RP7004123 | 1031 | 638 . . . 1516 | 3219 |
| NT2RP7005118 | 1032 | 108 . . . 3179 | 3220 |
| NT2RP7005529 | 1033 | 156 . . . 2531 | 3221 |
| NT2RP7005846 | 1034 | 1536 . . . 2180 | 3222 |
| NT2RP7009030 | 1035 | 732 . . . 1199 | 3223 |
| NT2RP7009147 | 1036 | 434 . . . >2686 | 3224 |
| NT2RP7009867 | 1037 | 880 . . . 1317 | 3225 |
| NT2RP7010128 | 1038 | 264 . . . 755 | 3226 |
| NT2RP7010599 | 1039 | 988 . . . 2160 | 3227 |
| NT2RP7011570 | 1040 | 1694 . . . 2017 | 3228 |
| NT2RP7013795 | 1041 | 284 . . . 1117 | 3229 |
| NT2RP7014005 | 1042 | 375 . . . 2135 | 3230 |
| NT2RP7015512 | 1043 | 2441 . . . 2863 | 3231 |
| NT2RP7017365 | 1044 | 5 . . . 349 | 3232 |
| NT2RP7017474 | 1045 | 234 . . . 1598 | 3233 |
| NT2RP7017546 | 1046 | 182 . . . 1018 | 3234 |
| NT2RP8000137 | 1047 | 1335 . . . 1673 | 3235 |
| NT2RP8000296 | 1048 | 911 . . . 2878 | 3236 |
| NT2RP8000483 | 1049 | 2903 . . . 4729 | 3237 |
| NTONG2000413 | 1050 | 331 . . . 1863 | 3238 |
| NTONG2003852 | 1051 | 341 . . . 1087 | 3239 |
| NTONG2005277 | 1052 | 293 . . . 2059 | 3240 |
| NTONG2005969 | 1053 | 1128 . . . 1436 | 3241 |
| NTONG2006354 | 1054 | 96 . . . 674 | 3242 |
| NTONG2007249 | 1055 | 439 . . . 1386 | 3243 |
| NTONG2007517 | 1056 | 34 . . . 930 | 3244 |
| NTONG2008088 | 1057 | 133 . . . 468 | 3245 |
| NTONG2008672 | 1058 | 37 . . . 2067 | 3246 |
| OCBBF1000254 | 1059 | 203 . . . 514 | 3247 |
| OCBBF2001794 | 1060 | 23 . . . 454 | 3248 |
| OCBBF2002124 | 1061 | 2496 . . . 2807 | 3249 |
| OCBBF2003819 | 1062 | 1788 . . . >2890 | 3250 |
| OCBBF2004826 | 1063 | 304 . . . >4404 | 3251 |
| OCBBF2004883 | 1064 | 1087 . . . 1803 | 3252 |
| OCBBF2005428 | 1065 | 1010 . . . 1369 | 3253 |
| OCBBF2006005 | 1066 | 107 . . . 2752 | 3254 |
| OCBBF2006058 | 1067 | 143 . . . 568 | 3255 |
| OCBBF2006151 | 1068 | 53 . . . 817 | 3256 |
| OCBBF2006567 | 1069 | 2568 . . . 2924 | 3257 |
| OCBBF2006764 | 1070 | 157 . . . 2571 | 3258 |
| OCBBF2007028 | 1071 | 1589 . . . 2572 | 3259 |
| OCBBF2007068 | 1072 | 108 . . . 3338 | 3260 |
| OCBBF2007114 | 1073 | 589 . . . 963 | 3261 |
| OCBBF2007428 | 1074 | 177 . . . 527 | 3262 |
| OCBBF2007478 | 1075 | 2014 . . . 2361 | 3263 |
| OCBBF2007610 | 1076 | 1229 . . . 1963 | 3264 |
| OCBBF2008770 | 1077 | 224 . . . 2677 | 3265 |
| OCBBF2009788 | 1078 | 18 . . . 371 | 3266 |
| OCBBF2009926 | 1079 | 734 . . . 1177 | 3267 |
| OCBBF2010140 | 1080 | 66 . . . 2459 | 3268 |
| OCBBF2010416 | 1081 | 1243 . . . >3531 | 3269 |
| OCBBF2017516 | 1082 | 459 . . . 860 | 3270 |
| OCBBF2019327 | 1083 | 221 . . . 553 | 3271 |
| OCBBF2019823 | 1084 | 798 . . . 1796 | 3272 |
| OCBBF2020343 | 1085 | 293 . . . 859 | 3273 |
| OCBBF2020453 | 1086 | 168 . . . 488 | 3274 |
| OCBBF2020639 | 1087 | 907 . . . 1248 | 3275 |
| OCBBF2020741 | 1088 | 962 . . . 1555 | 3276 |
| OCBBF2020801 | 1089 | 1544 . . . 2017 | 3277 |
| OCBBF2020838 | 1090 | 593 . . . 1819 | 3278 |
| OCBBF2021020 | 1091 | 1232 . . . 2206 | 3279 |
| OCBBF2021286 | 1092 | 86 . . . 787 | 3280 |
| OCBBF2021323 | 1093 | 8 . . . 538 | 3281 |
| OCBBF2021788 | 1094 | 19 . . . 2793 | 3282 |
| OCBBF2022351 | 1095 | 157 . . . 1443 | 3283 |
| OCBBF2022574 | 1096 | 362 . . . 769 | 3284 |
| OCBBF2023162 | 1097 | 222 . . . 536 | 3285 |
| OCBBF2023643 | 1098 | 1236 . . . 1658 | 3286 |
| OCBBF2024719 | 1099 | 658 . . . 999 | 3287 |
| OCBBF2024781 | 1100 | 358 . . . 987 | 3288 |
| OCBBF2024850 | 1101 | 329 . . . 652 | 3289 |
| OCBBF2025028 | 1102 | 431 . . . 1504 | 3290 |
| OCBBF2025458 | 1103 | 10 . . . 486 | 3291 |
| OCBBF2025527 | 1104 | 627 . . . 980 | 3292 |
| OCBBF2025730 | 1105 | 614 . . . 949 | 3293 |
| OCBBF2026645 | 1106 | 1905 . . . 2216 | 3294 |
| OCBBF2027423 | 1107 | 25 . . . 360 | 3295 |
| OCBBF2027478 | 1108 | 1362 . . . 1796 | 3296 |
| OCBBF2028173 | 1109 | 764 . . . 1723 | 3297 |
| OCBBF2028935 | 1110 | 76 . . . 432 | 3298 |
| OCBBF2029901 | 1111 | 357 . . . 836 | 3299 |
| OCBBF2030354 | 1112 | 261 . . . 1646 | 3300 |
| OCBBF2030517 | 1113 | 1378 . . . 1842 | 3301 |
| OCBBF2030574 | 1114 | 54 . . . 605 | 3302 |
| OCBBF2030708 | 1115 | 409 . . . 2361 | 3303 |
| OCBBF2031167 | 1116 | 31 . . . >2709 | 3304 |
| OCBBF2031366 | 1117 | 1696 . . . 2055 | 3305 |
| OCBBF2032590 | 1118 | 395 . . . 1021 | 3306 |
| OCBBF2032599 | 1119 | 30 . . . 371 | 3307 |
| OCBBF2032611 | 1120 | 1 . . . 354 | 3308 |
| OCBBF2032671 | 1121 | 960 . . . 1355 | 3309 |
| OCBBF2033869 | 1122 | 882 . . . 1355 | 3310 |
| OCBBF2035110 | 1123 | 1534 . . . 3426 | 3311 |
| OCBBF2035214 | 1124 | 342 . . . 644 | 3312 |
| OCBBF2035564 | 1125 | 321 . . . 2228 | 3313 |
| OCBBF2035885 | 1126 | 76 . . . 420 | 3314 |
| OCBBF2035916 | 1127 | 1859 . . . 2524 | 3315 |
| OCBBF2036476 | 1128 | 1103 . . . 1873 | 3316 |
| OCBBF2036743 | 1129 | 526 . . . 2445 | 3317 |
| OCBBF2037068 | 1130 | 318 . . . 1067 | 3318 |
| OCBBF2037340 | 1131 | 526 . . . >3404 | 3319 |
| OCBBF2037398 | 1132 | 286 . . . 651 | 3320 |
| OCBBF2037547 | 1133 | 54 . . . 3860 | 3321 |
| OCBBF2037598 | 1134 | 1515 . . . >3057 | 3322 |
| OCBBF2037638 | 1135 | 754 . . . 2043 | 3323 |
| OCBBF2038317 | 1136 | 183 . . . 3575 | 3324 |
| OCBBF3000296 | 1137 | 1505 . . . 2095 | 3325 |
| OCBBF3000483 | 1138 | 37 . . . 471 | 3326 |
| OCBBF3002553 | 1139 | 27 . . . 581 | 3327 |
| OCBBF3002600 | 1140 | 2661 . . . 3005 | 3328 |
| OCBBF3003320 | 1141 | 20 . . . 1000 | 3329 |
| OCBBF3003592 | 1142 | 86 . . . 2827 | 3330 |
| OCBBF3004314 | 1143 | 687 . . . 1187 | 3331 |
| OCBBF3006802 | 1144 | 236 . . . 625 | 3332 |
| OCBBF3007516 | 1145 | 1479 . . . 1829 | 3333 |
| OCBBF3008230 | 1146 | 2923 . . . >3428 | 3334 |
| OCBBF3009279 | 1147 | 34 . . . 1065 | 3335 |
| PEBLM2000170 | 1148 | 434 . . . 811 | 3336 |
| PEBLM2000338 | 1149 | 1419 . . . 1865 | 3337 |
| PEBLM2001465 | 1150 | 856 . . . 1374 | 3338 |
| PEBLM2001488 | 1151 | 325 . . . 732 | 3339 |
| PEBLM2002594 | 1152 | 688 . . . 2121 | 3340 |
| PEBLM2002749 | 1153 | 275 . . . 820 | 3341 |
| PEBLM2002887 | 1154 | 948 . . . 1301 | 3342 |
| PEBLM2004497 | 1155 | 502 . . . 888 | 3343 |
| PEBLM2004666 | 1156 | 139 . . . 2397 | 3344 |
| PEBLM2005183 | 1157 | 9 . . . 2426 | 3345 |
| PEBLM2005697 | 1158 | 408 . . . 734 | 3346 |
| PEBLM2006113 | 1159 | 29 . . . 421 | 3347 |
| PEBLM2007112 | 1160 | 243 . . . 704 | 3348 |
| PEBLM2007140 | 1161 | 267 . . . 629 | 3349 |
| PEBLM2007834 | 1162 | 177 . . . 539 | 3350 |
| PERIC1000147 | 1163 | 83 . . . >2669 | 3351 |
| PERIC2000889 | 1164 | 1483 . . . 2079 | 3352 |
| PERIC2000914 | 1165 | 1557 . . . 1901 | 3353 |
| PERIC2001227 | 1166 | 936 . . . 1253 | 3354 |
| PERIC2001228 | 1167 | 2 . . . 754 | 3355 |
| PERIC2002766 | 1168 | 64 . . . 384 | 3356 |
| PERIC2003090 | 1169 | 158 . . . 802 | 3357 |
| PERIC2003452 | 1170 | 1555 . . . 1890 | 3358 |
| PERIC2003699 | 1171 | 100 . . . 435 | 3359 |
| PERIC2003720 | 1172 | 640 . . . 1512 | 3360 |
| PERIC2003834 | 1173 | 103 . . . 531 | 3361 |
| PERIC2004028 | 1174 | 751 . . . 1212 | 3362 |
| PERIC2004259 | 1175 | 1686 . . . 2042 | 3363 |
| PERIC2004379 | 1176 | 55 . . . 483 | 3364 |
| PERIC2004429 | 1177 | 12 . . . 377 | 3365 |
| PERIC2004909 | 1178 | 32 . . . 334 | 3366 |
| PERIC2005347 | 1179 | 26 . . . 499 | 3367 |
| PERIC2005370 | 1180 | 13 . . . 1041 | 3368 |
| PERIC2006035 | 1181 | 1 . . . 1134 | 3369 |
| PERIC2007914 | 1182 | 1492 . . . 2073 | 3370 |
| PERIC2008385 | 1183 | 348 . . . 722 | 3371 |
| PERIC2009086 | 1184 | 202 . . . 1749 | 3372 |
| PLACE5000001 | 1185 | 2536 . . . 3036 | 3373 |
| PLACE5000171 | 1186 | 334 . . . 2940 | 3374 |
| PLACE5000260 | 1187 | 242 . . . 742 | 3375 |
| PLACE5000282 | 1188 | 60 . . . 1739 | 3376 |
| PLACE6001185 | 1189 | 765 . . . 1337 | 3377 |
| PLACE6009006 | 1190 | 1018 . . . 1518 | 3378 |
| PLACE6012574 | 1191 | 368 . . . 1984 | 3379 |
| PLACE6019385 | 1192 | 617 . . . 1360 | 3380 |
| PLACE6019932 | 1193 | 20 . . . 847 | 3381 |
| PLACE6020031 | 1194 | 228 . . . 1586 | 3382 |
| PLACE7000514 | 1195 | 17 . . . 1246 | 3383 |
| PLACE7001022 | 1196 | 1733 . . . 2197 | 3384 |
| PLACE7001936 | 1197 | 272 . . . 697 | 3385 |
| PLACE7002641 | 1198 | 1254 . . . 2264 | 3386 |
| PLACE7006051 | 1199 | 145 . . . >3409 | 3387 |
| PLACE7008431 | 1200 | 223 . . . 1281 | 3388 |
| PLACE7008623 | 1201 | 1304 . . . 1762 | 3389 |
| PROST1000184 | 1202 | 734 . . . 1123 | 3390 |
| PROST1000528 | 1203 | 428 . . . 838 | 3391 |
| PROST1000559 | 1204 | 149 . . . 856 | 3392 |
| PROST2003428 | 1205 | 1031 . . . 1387 | 3393 |
| PROST2008993 | 1206 | 362 . . . 2008 | 3394 |
| PROST2015243 | 1207 | 1401 . . . 1802 | 3395 |
| PROST2016462 | 1208 | 435 . . . 2081 | 3396 |
| PROST2017367 | 1209 | 72 . . . 428 | 3397 |
| PROST2017413 | 1210 | 1163 . . . 1510 | 3398 |
| PROST2017700 | 1211 | 1337 . . . 1645 | 3399 |
| PROST2018030 | 1212 | 884 . . . 1255 | 3400 |
| PROST2018090 | 1213 | 117 . . . 1448 | 3401 |
| PROST2018511 | 1214 | 136 . . . 1803 | 3402 |
| PROST2018902 | 1215 | 288 . . . 653 | 3403 |
| PROST2018922 | 1216 | 556 . . . 888 | 3404 |
| PROST2019296 | 1217 | 415 . . . 777 | 3405 |
| PROST2019781 | 1218 | 139 . . . 441 | 3406 |
| PUAEN2002489 | 1219 | 410 . . . 1186 | 3407 |
| PUAEN2002616 | 1220 | 1790 . . . 2389 | 3408 |
| PUAEN2003079 | 1221 | 438 . . . 812 | 3409 |
| PUAEN2005588 | 1222 | 134 . . . 580 | 3410 |
| PUAEN2005930 | 1223 | 1673 . . . 3256 | 3411 |
| PUAEN2006328 | 1224 | 166 . . . 2064 | 3412 |
| PUAEN2006701 | 1225 | 277 . . . 837 | 3413 |
| PUAEN2007044 | 1226 | 51 . . . 521 | 3414 |
| PUAEN2007785 | 1227 | 23 . . . 1144 | 3415 |
| PUAEN2009174 | 1228 | 4 . . . 2547 | 3416 |
| PUAEN2009655 | 1229 | 237 . . . 2198 | 3417 |
| PUAEN2009795 | 1230 | 121 . . . 1716 | 3418 |
| PUAEN2009852 | 1231 | 953 . . . 1291 | 3419 |
| RECTM2000433 | 1232 | 33 . . . 536 | 3420 |
| RECTM2001347 | 1233 | 638 . . . 1609 | 3421 |
| SKMUS2000757 | 1234 | 289 . . . 606 | 3422 |
| SKMUS2003074 | 1235 | 291 . . . 692 | 3423 |
| SKMUS2004047 | 1236 | 628 . . . 1047 | 3424 |
| SKMUS2006394 | 1237 | 111 . . . 1469 | 3425 |
| SKNMC1000124 | 1238 | 194 . . . 1390 | 3426 |
| SKNMC2002402 | 1239 | 107 . . . 466 | 3427 |
| SKNMC2004457 | 1240 | 1304 . . . 1750 | 3428 |
| SKNMC2004643 | 1241 | 26 . . . 697 | 3429 |
| SKNMC2005772 | 1242 | 1187 . . . 1585 | 3430 |
| SKNMC2006998 | 1243 | 97 . . . 558 | 3431 |
| SKNMC2007504 | 1244 | 27 . . . 1190 | 3432 |
| SKNMC2007961 | 1245 | 170 . . . 472 | 3433 |
| SKNMC2009450 | 1246 | 103 . . . 411 | 3434 |
| SKNSH2000482 | 1247 | 1184 . . . 1561 | 3435 |
| SKNSH2009991 | 1248 | 1073 . . . 1378 | 3436 |
| SKNSH2010015 | 1249 | 1308 . . . 1613 | 3437 |
| SMINT1000192 | 1250 | 49 . . . 810 | 3438 |
| SMINT2001818 | 1251 | 616 . . . 1158 | 3439 |
| SMINT2002743 | 1252 | 995 . . . 1375 | 3440 |
| SMINT2006641 | 1253 | 1820 . . . 2185 | 3441 |
| SMINT2007391 | 1254 | 229 . . . 1197 | 3442 |
| SMINT2009902 | 1255 | 98 . . . 901 | 3443 |
| SMINT2010076 | 1256 | 81 . . . 1574 | 3444 |
| SMINT2010897 | 1257 | 230 . . . 535 | 3445 |
| SMINT2011311 | 1258 | 594 . . . 1121 | 3446 |
| SMINT2011888 | 1259 | 80 . . . 1543 | 3447 |
| SMINT2015787 | 1260 | 48 . . . 551 | 3448 |
| SPLEN2001599 | 1261 | 20 . . . 1297 | 3449 |
| SPLEN2002147 | 1262 | 607 . . . 1068 | 3450 |
| SPLEN2002467 | 1263 | 256 . . . 1566 | 3451 |
| SPLEN2002707 | 1264 | 234 . . . 545 | 3452 |
| SPLEN2006122 | 1265 | 162 . . . 1121 | 3453 |
| SPLEN2009548 | 1266 | 349 . . . 723 | 3454 |
| SPLEN2010912 | 1267 | 1159 . . . 2286 | 3455 |
| SPLEN2011422 | 1268 | 79 . . . 1008 | 3456 |
| SPLEN2012624 | 1269 | 1077 . . . 1784 | 3457 |
| SPLEN2012889 | 1270 | 1647 . . . 2018 | 3458 |
| SPLEN2014946 | 1271 | 161 . . . 547 | 3459 |
| SPLEN2015158 | 1272 | 567 . . . 947 | 3460 |
| SPLEN2015267 | 1273 | 81 . . . 1646 | 3461 |
| SPLEN2015679 | 1274 | 517 . . . >2171 | 3462 |
| SPLEN2016554 | 1275 | 200 . . . 3343 | 3463 |
| SPLEN2016863 | 1276 | 276 . . . 1352 | 3464 |
| SPLEN2017104 | 1277 | 264 . . . 1274 | 3465 |
| SPLEN2021701 | 1278 | 23 . . . 910 | 3466 |
| SPLEN2023733 | 1279 | 158 . . . 1264 | 3467 |
| SPLEN2023791 | 1280 | 144 . . . 746 | 3468 |
| SPLEN2024127 | 1281 | 198 . . . 584 | 3469 |
| SPLEN2025491 | 1282 | 1056 . . . 1466 | 3470 |
| SPLEN2027268 | 1283 | 1655 . . . 2161 | 3471 |
| SPLEN2028844 | 1284 | 271 . . . 840 | 3472 |
| SPLEN2028914 | 1285 | 253 . . . 855 | 3473 |
| SPLEN2029051 | 1286 | 234 . . . 596 | 3474 |
| SPLEN2029176 | 1287 | 531 . . . 998 | 3475 |
| SPLEN2029522 | 1288 | 1430 . . . 1771 | 3476 |
| SPLEN2029683 | 1289 | 1308 . . . 1682 | 3477 |
| SPLEN2029727 | 1290 | 2754 . . . 3068 | 3478 |
| SPLEN2029912 | 1291 | 216 . . . 653 | 3479 |
| SPLEN2030335 | 1292 | 382 . . . 1104 | 3480 |
| SPLEN2030479 | 1293 | 567 . . . 1061 | 3481 |
| SPLEN2031125 | 1294 | 24 . . . 407 | 3482 |
| SPLEN2031424 | 1295 | 641 . . . 1138 | 3483 |
| SPLEN2031547 | 1296 | 428 . . . 1645 | 3484 |
| SPLEN2031724 | 1297 | 385 . . . 765 | 3485 |
| SPLEN2031780 | 1298 | 78 . . . 1067 | 3486 |
| SPLEN2032154 | 1299 | 244 . . . 735 | 3487 |
| SPLEN2032321 | 1300 | 1893 . . . 2222 | 3488 |
| SPLEN2032813 | 1301 | 799 . . . 1134 | 3489 |
| SPLEN2033098 | 1302 | 1452 . . . 2051 | 3490 |
| SPLEN2033153 | 1303 | 109 . . . 471 | 3491 |
| SPLEN2033539 | 1304 | 1359 . . . 1682 | 3492 |
| SPLEN2033921 | 1305 | 2125 . . . 2478 | 3493 |
| SPLEN2034021 | 1306 | 147 . . . 482 | 3494 |
| SPLEN2034081 | 1307 | 536 . . . 1042 | 3495 |
| SPLEN2034678 | 1308 | 61 . . . 495 | 3496 |
| SPLEN2034781 | 1309 | 629 . . . 1618 | 3497 |
| SPLEN2036103 | 1310 | 1088 . . . 1627 | 3498 |
| SPLEN2036326 | 1311 | 1062 . . . 1973 | 3499 |
| SPLEN2036712 | 1312 | 179 . . . 529 | 3500 |
| SPLEN2036821 | 1313 | 932 . . . 1276 | 3501 |
| SPLEN2036932 | 1314 | 1724 . . . 2170 | 3502 |
| SPLEN2037194 | 1315 | 185 . . . 1984 | 3503 |
| SPLEN2037580 | 1316 | 1852 . . . 2301 | 3504 |
| SPLEN2037630 | 1317 | 1219 . . . 1542 | 3505 |
| SPLEN2037722 | 1318 | 71 . . . 1066 | 3506 |
| SPLEN2038055 | 1319 | 1738 . . . 2220 | 3507 |
| SPLEN2038180 | 1320 | 8 . . . 496 | 3508 |
| SPLEN2038345 | 1321 | 356 . . . 793 | 3509 |
| SPLEN2038407 | 1322 | 185 . . . 2005 | 3510 |
| SPLEN2039697 | 1323 | 791 . . . 1105 | 3511 |
| SPLEN2039936 | 1324 | 71 . . . 382 | 3512 |
| SPLEN2040222 | 1325 | 106 . . . 840 | 3513 |
| SPLEN2041304 | 1326 | 72 . . . 398 | 3514 |
| SPLEN2041310 | 1327 | 1402 . . . 1734 | 3515 |
| SPLEN2041645 | 1328 | 1624 . . . 1998 | 3516 |
| SPLEN2041720 | 1329 | 57 . . . 494 | 3517 |
| SPLEN2041977 | 1330 | 72 . . . 374 | 3518 |
| SPLEN2042303 | 1331 | 480 . . . 791 | 3519 |
| SPLEN2042598 | 1332 | 70 . . . 387 | 3520 |
| STOMA1000189 | 1333 | 1050 . . . 1436 | 3521 |
| STOMA2003444 | 1334 | 1664 . . . 1975 | 3522 |
| STOMA2004294 | 1335 | 38 . . . 607 | 3523 |
| STOMA2004925 | 1336 | 854 . . . 1285 | 3524 |
| STOMA2008546 | 1337 | 119 . . . 1060 | 3525 |
| SYNOV1000374 | 1338 | 204 . . . 659 | 3526 |
| SYNOV2005216 | 1339 | 1201 . . . >2283 | 3527 |
| SYNOV2005448 | 1340 | 1261 . . . 1653 | 3528 |
| SYNOV2005817 | 1341 | 412 . . . 1395 | 3529 |
| SYNOV2006430 | 1342 | 232 . . . 780 | 3530 |
| SYNOV2007965 | 1343 | 83 . . . 1375 | 3531 |
| SYNOV2012326 | 1344 | 30 . . . 476 | 3532 |
| SYNOV2014400 | 1345 | 389 . . . 988 | 3533 |
| SYNOV2016124 | 1346 | 1174 . . . 1476 | 3534 |
| SYNOV2017055 | 1347 | 981 . . . 1511 | 3535 |
| SYNOV2018921 | 1348 | 126 . . . 737 | 3536 |
| SYNOV2021320 | 1349 | 182 . . . 1792 | 3537 |
| SYNOV3000231 | 1350 | 81 . . . 1499 | 3538 |
| SYNOV3000302 | 1351 | 80 . . . 1423 | 3539 |
| SYNOV4000472 | 1352 | 103 . . . 540 | 3540 |
| SYNOV4000706 | 1353 | 121 . . . 2538 | 3541 |
| SYNOV4001326 | 1354 | 196 . . . 1086 | 3542 |
| SYNOV4001395 | 1355 | 959 . . . 2092 | 3543 |
| SYNOV4002346 | 1356 | 150 . . . 3797 | 3544 |
| SYNOV4002392 | 1357 | 148 . . . 951 | 3545 |
| SYNOV4002883 | 1358 | 1686 . . . 2240 | 3546 |
| SYNOV4003322 | 1359 | 2461 . . . 2859 | 3547 |
| SYNOV4004184 | 1360 | 2542 . . . 2889 | 3548 |
| SYNOV4004741 | 1361 | 776 . . . 1237 | 3549 |
| SYNOV4004823 | 1362 | 3332 . . . 3715 | 3550 |
| SYNOV4004914 | 1363 | 2753 . . . 3106 | 3551 |
| SYNOV4006256 | 1364 | 182 . . . 493 | 3552 |
| SYNOV4007012 | 1365 | 546 . . . 1007 | 3553 |
| SYNOV4007215 | 1366 | 78 . . . >3375 | 3554 |
| SYNOV4007360 | 1367 | 567 . . . 1364 | 3555 |
| SYNOV4007430 | 1368 | 47 . . . 448 | 3556 |
| SYNOV4007521 | 1369 | 6 . . . 935 | 3557 |
| SYNOV4007553 | 1370 | 1405 . . . 3759 | 3558 |
| SYNOV4007671 | 1371 | 548 . . . 1417 | 3559 |
| SYNOV4008336 | 1372 | 825 . . . 1256 | 3560 |
| SYNOV4008440 | 1373 | 115 . . . 3804 | 3561 |
| T1ESE2000116 | 1374 | 1455 . . . 1769 | 3562 |
| TBAES2001171 | 1375 | 871 . . . 1845 | 3563 |
| TBAES2001220 | 1376 | 155 . . . 547 | 3564 |
| TBAES2001229 | 1377 | 1496 . . . 1861 | 3565 |
| TBAES2001258 | 1378 | 369 . . . 677 | 3566 |
| TBAES2001492 | 1379 | 630 . . . 1241 | 3567 |
| TBAES2001751 | 1380 | 185 . . . 592 | 3568 |
| TBAES2002197 | 1381 | 5 . . . 1609 | 3569 |
| TBAES2003550 | 1382 | 1130 . . . 1549 | 3570 |
| TBAES2004055 | 1383 | 898 . . . 1935 | 3571 |
| TBAES2005157 | 1384 | 119 . . . 1573 | 3572 |
| TBAES2005543 | 1385 | 1197 . . . 1694 | 3573 |
| TBAES2006568 | 1386 | 169 . . . 996 | 3574 |
| TBAES2007964 | 1387 | 72 . . . 449 | 3575 |
| TCERX2000613 | 1388 | 427 . . . 1410 | 3576 |
| TCOLN2002278 | 1389 | 1064 . . . 1432 | 3577 |
| TESOP1000127 | 1390 | 358 . . . 708 | 3578 |
| TESOP2000801 | 1391 | 707 . . . 1216 | 3579 |
| TESOP2001122 | 1392 | 207 . . . 998 | 3580 |
| TESOP2001166 | 1393 | 290 . . . 1612 | 3581 |
| TESOP2001345 | 1394 | 33 . . . 350 | 3582 |
| TESOP2001605 | 1395 | 746 . . . 1651 | 3583 |
| TESOP2001818 | 1396 | 491 . . . 1456 | 3584 |
| TESOP2001849 | 1397 | 17 . . . 853 | 3585 |
| TESOP2001865 | 1398 | 6 . . . 368 | 3586 |
| TESOP2001953 | 1399 | 372 . . . 1871 | 3587 |
| TESOP2002273 | 1400 | 163 . . . 558 | 3588 |
| TESOP2002451 | 1401 | 99 . . . 1157 | 3589 |
| TESOP2002489 | 1402 | 18 . . . 365 | 3590 |
| TESOP2002539 | 1403 | 76 . . . 459 | 3591 |
| TESOP2002950 | 1404 | 1262 . . . 1747 | 3592 |
| TESOP2003273 | 1405 | 691 . . . 1032 | 3593 |
| TESOP2003753 | 1406 | 97 . . . 399 | 3594 |
| TESOP2004114 | 1407 | 510 . . . 1766 | 3595 |
| TESOP2005285 | 1408 | 510 . . . 1181 | 3596 |
| TESOP2005485 | 1409 | 62 . . . 994 | 3597 |
| TESOP2005579 | 1410 | 755 . . . 1729 | 3598 |
| TESOP2006041 | 1411 | 315 . . . 1538 | 3599 |
| TESOP2006060 | 1412 | 606 . . . 1040 | 3600 |
| TESOP2006068 | 1413 | 930 . . . 1382 | 3601 |
| TESOP2006670 | 1414 | 744 . . . 1235 | 3602 |
| TESOP2006746 | 1415 | 1158 . . . 1655 | 3603 |
| TESOP2007052 | 1416 | 28 . . . 351 | 3604 |
| TESOP2007262 | 1417 | 870 . . . 2078 | 3605 |
| TESOP2007636 | 1418 | 1053 . . . 1403 | 3606 |
| TESOP2007688 | 1419 | 210 . . . 863 | 3607 |
| TESOP2009121 | 1420 | 39 . . . >2370 | 3608 |
| TESOP2009555 | 1421 | 741 . . . 1565 | 3609 |
| TESTI1000257 | 1422 | 377 . . . 1870 | 3610 |
| TESTI1000319 | 1423 | 107 . . . 2773 | 3611 |
| TESTI1000330 | 1424 | 271 . . . 576 | 3612 |
| TESTI1000348 | 1425 | 802 . . . 1251 | 3613 |
| TESTI1000390 | 1426 | 1642 . . . 3522 | 3614 |
| TESTI1000491 | 1427 | 33 . . . 428 | 3615 |
| TESTI1000545 | 1428 | 836 . . . 3625 | 3616 |
| TESTI2000443 | 1429 | 682 . . . 2265 | 3617 |
| TESTI2000644 | 1430 | 864 . . . 1211 | 3618 |
| TESTI2002036 | 1431 | 114 . . . 1700 | 3619 |
| TESTI2002618 | 1432 | 42 . . . 881 | 3620 |
| TESTI2002928 | 1433 | 272 . . . 757 | 3621 |
| TESTI2003347 | 1434 | 285 . . . 1829 | 3622 |
| TESTI2003573 | 1435 | 326 . . . 1363 | 3623 |
| TESTI2004215 | 1436 | 300 . . . 2456 | 3624 |
| TESTI2004700 | 1437 | 614 . . . 1273 | 3625 |
| TESTI2005376 | 1438 | 412 . . . 2103 | 3626 |
| TESTI2005610 | 1439 | 426 . . . 1964 | 3627 |
| TESTI2005739 | 1440 | 514 . . . 1848 | 3628 |
| TESTI2005986 | 1441 | 204 . . . 551 | 3629 |
| TESTI2006041 | 1442 | 697 . . . 1047 | 3630 |
| TESTI2006643 | 1443 | 812 . . . 1882 | 3631 |
| TESTI2006648 | 1444 | 204 . . . 1919 | 3632 |
| TESTI2009474 | 1445 | 283 . . . 1536 | 3633 |
| TESTI2009477 | 1446 | 1176 . . . 1649 | 3634 |
| TESTI2009511 | 1447 | 154 . . . 663 | 3635 |
| TESTI2009812 | 1448 | 910 . . . 1308 | 3636 |
| TESTI2010400 | 1449 | 1814 . . . 2224 | 3637 |
| TESTI2013381 | 1450 | 351 . . . 695 | 3638 |
| TESTI2013382 | 1451 | 1332 . . . 1826 | 3639 |
| TESTI2014716 | 1452 | 64 . . . 1443 | 3640 |
| TESTI2014843 | 1453 | 148 . . . 1866 | 3641 |
| TESTI2016046 | 1454 | 152 . . . 2173 | 3642 |
| TESTI2017727 | 1455 | 83 . . . 541 | 3643 |
| TESTI2018838 | 1456 | 647 . . . 1147 | 3644 |
| TESTI2019042 | 1457 | 130 . . . 1011 | 3645 |
| TESTI2019648 | 1458 | 1044 . . . 1463 | 3646 |
| TESTI2023254 | 1459 | 206 . . . 1972 | 3647 |
| TESTI2023599 | 1460 | 200 . . . 565 | 3648 |
| TESTI2024567 | 1461 | 1072 . . . >1878 | 3649 |
| TESTI2026505 | 1462 | 69 . . . 1691 | 3650 |
| TESTI2027019 | 1463 | 608 . . . 1342 | 3651 |
| TESTI2031529 | 1464 | 386 . . . 1819 | 3652 |
| TESTI2034520 | 1465 | 59 . . . 1519 | 3653 |
| TESTI2034749 | 1466 | 973 . . . 1503 | 3654 |
| TESTI2034767 | 1467 | 374 . . . 1786 | 3655 |
| TESTI2034953 | 1468 | 703 . . . 1113 | 3656 |
| TESTI2034997 | 1469 | 179 . . . 547 | 3657 |
| TESTI2035107 | 1470 | 126 . . . 533 | 3658 |
| TESTI2035997 | 1471 | 826 . . . 1140 | 3659 |
| TESTI2036513 | 1472 | 1569 . . . 1895 | 3660 |
| TESTI2036684 | 1473 | 297 . . . 839 | 3661 |
| TESTI2037643 | 1474 | 3 . . . 599 | 3662 |
| TESTI2040018 | 1475 | 441 . . . >1825 | 3663 |
| TESTI2042450 | 1476 | 212 . . . 544 | 3664 |
| TESTI2044796 | 1477 | 913 . . . 1692 | 3665 |
| TESTI2044833 | 1478 | 265 . . . 633 | 3666 |
| TESTI2045920 | 1479 | 15 . . . 758 | 3667 |
| TESTI2045983 | 1480 | 103 . . . 609 | 3668 |
| TESTI2046347 | 1481 | 465 . . . 1022 | 3669 |
| TESTI2047071 | 1482 | 596 . . . 1012 | 3670 |
| TESTI2048465 | 1483 | 755 . . . 1315 | 3671 |
| TESTI2048603 | 1484 | 122 . . . 490 | 3672 |
| TESTI2048898 | 1485 | 6 . . . 374 | 3673 |
| TESTI2049206 | 1486 | 677 . . . 1003 | 3674 |
| TESTI2049246 | 1487 | 296 . . . 658 | 3675 |
| TESTI2049277 | 1488 | 765 . . . 1130 | 3676 |
| TESTI2049422 | 1489 | 69 . . . 941 | 3677 |
| TESTI2049452 | 1490 | 28 . . . 381 | 3678 |
| TESTI2049469 | 1491 | 313 . . . 1341 | 3679 |
| TESTI2049576 | 1492 | 1030 . . . 1773 | 3680 |
| TESTI2049857 | 1493 | 816 . . . 2057 | 3681 |
| TESTI2050137 | 1494 | 6 . . . 1283 | 3682 |
| TESTI2050681 | 1495 | 186 . . . 605 | 3683 |
| TESTI2050987 | 1496 | 313 . . . 1104 | 3684 |
| TESTI2051279 | 1497 | 338 . . . 922 | 3685 |
| TESTI2051488 | 1498 | 665 . . . 1171 | 3686 |
| TESTI2051543 | 1499 | 925 . . . 1491 | 3687 |
| TESTI2051767 | 1500 | 568 . . . 945 | 3688 |
| TESTI2051806 | 1501 | 60 . . . 1091 | 3689 |
| TESTI2051867 | 1502 | 779 . . . 1780 | 3690 |
| TESTI2052211 | 1503 | 264 . . . 1358 | 3691 |
| TESTI2052693 | 1504 | 929 . . . 1954 | 3692 |
| TESTI2052698 | 1505 | 451 . . . 1011 | 3693 |
| TESTI2052822 | 1506 | 103 . . . 459 | 3694 |
| TESTI2053242 | 1507 | 86 . . . 574 | 3695 |
| TESTI2053399 | 1508 | 81 . . . 503 | 3696 |
| TESTI2053526 | 1509 | 116 . . . 502 | 3697 |
| TESTI2053621 | 1510 | 977 . . . 1582 | 3698 |
| TESTI4000014 | 1511 | 30 . . . 4214 | 3699 |
| TESTI4000068 | 1512 | 1820 . . . 2359 | 3700 |
| TESTI4000079 | 1513 | 1263 . . . 3458 | 3701 |
| TESTI4000209 | 1514 | 24 . . . 1049 | 3702 |
| TESTI4000215 | 1515 | 230 . . . 922 | 3703 |
| TESTI4000250 | 1516 | 56 . . . 496 | 3704 |
| TESTI4000288 | 1517 | 546 . . . 1082 | 3705 |
| TESTI4000349 | 1518 | 1259 . . . 3469 | 3706 |
| TESTI4000462 | 1519 | 886 . . . 1497 | 3707 |
| TESTI4000530 | 1520 | 346 . . . 879 | 3708 |
| TESTI4000724 | 1521 | 110 . . . 1639 | 3709 |
| TESTI4000970 | 1522 | 458 . . . 2476 | 3710 |
| TESTI4001100 | 1523 | 55 . . . 723 | 3711 |
| TESTI4001106 | 1524 | 453 . . . 2180 | 3712 |
| TESTI4001148 | 1525 | 94 . . . 1947 | 3713 |
| TESTI4001176 | 1526 | 636 . . . 1178 | 3714 |
| TESTI4001201 | 1527 | 106 . . . 441 | 3715 |
| TESTI4001206 | 1528 | 500 . . . 916 | 3716 |
| TESTI4001527 | 1529 | 710 . . . 1468 | 3717 |
| TESTI4001561 | 1530 | 2780 . . . >3955 | 3718 |
| TESTI4001665 | 1531 | 4092 . . . 4538 | 3719 |
| TESTI4001923 | 1532 | 200 . . . 658 | 3720 |
| TESTI4002290 | 1533 | 1054 . . . 1497 | 3721 |
| TESTI4002491 | 1534 | 1553 . . . 1873 | 3722 |
| TESTI4002552 | 1535 | 2112 . . . 3710 | 3723 |
| TESTI4002647 | 1536 | 190 . . . >3607 | 3724 |
| TESTI4002703 | 1537 | 1531 . . . 2484 | 3725 |
| TESTI4002754 | 1538 | 157 . . . 600 | 3726 |
| TESTI4002878 | 1539 | 150 . . . 650 | 3727 |
| TESTI4004200 | 1540 | 4 . . . 498 | 3728 |
| TESTI4005628 | 1541 | 158 . . . 487 | 3729 |
| TESTI4005805 | 1542 | 4 . . . 468 | 3730 |
| TESTI4005857 | 1543 | 3018 . . . 3860 | 3731 |
| TESTI4005961 | 1544 | 3600 . . . 3911 | 3732 |
| TESTI4006053 | 1545 | 163 . . . 480 | 3733 |
| TESTI4006079 | 1546 | 1652 . . . 3514 | 3734 |
| TESTI4006112 | 1547 | 799 . . . 1635 | 3735 |
| TESTI4006137 | 1548 | 235 . . . 798 | 3736 |
| TESTI4006148 | 1549 | 358 . . . 1035 | 3737 |
| TESTI4006219 | 1550 | 138 . . . 479 | 3738 |
| TESTI4006326 | 1551 | 1194 . . . 2537 | 3739 |
| TESTI4006393 | 1552 | 2691 . . . 3194 | 3740 |
| TESTI4006412 | 1553 | 83 . . . 427 | 3741 |
| TESTI4006420 | 1554 | 1850 . . . 2674 | 3742 |
| TESTI4006546 | 1555 | 286 . . . 3543 | 3743 |
| TESTI4006802 | 1556 | 95 . . . 3277 | 3744 |
| TESTI4006819 | 1557 | 1355 . . . 1681 | 3745 |
| TESTI4007064 | 1558 | 5 . . . 4678 | 3746 |
| TESTI4007163 | 1559 | 2933 . . . 3799 | 3747 |
| TESTI4007203 | 1560 | 79 . . . 420 | 3748 |
| TESTI4007239 | 1561 | 36 . . . 4373 | 3749 |
| TESTI4007373 | 1562 | 77 . . . 493 | 3750 |
| TESTI4007382 | 1563 | 32 . . . 2785 | 3751 |
| TESTI4007404 | 1564 | 82 . . . 4038 | 3752 |
| TESTI4007489 | 1565 | 3867 . . . 4589 | 3753 |
| TESTI4007775 | 1566 | 1493 . . . 1870 | 3754 |
| TESTI4007778 | 1567 | 665 . . . 3337 | 3755 |
| TESTI4007799 | 1568 | 1200 . . . 2039 | 3756 |
| TESTI4007810 | 1569 | 1972 . . . 2739 | 3757 |
| TESTI4008007 | 1570 | 1375 . . . 1983 | 3758 |
| TESTI4008018 | 1571 | 2012 . . . 2338 | 3759 |
| TESTI4008050 | 1572 | 968 . . . 2101 | 3760 |
| TESTI4008219 | 1573 | 2148 . . . 2618 | 3761 |
| TESTI4008401 | 1574 | 288 . . . 731 | 3762 |
| TESTI4008429 | 1575 | 2906 . . . 4009 | 3763 |
| TESTI4008573 | 1576 | 199 . . . 528 | 3764 |
| TESTI4008797 | 1577 | 2518 . . . 3342 | 3765 |
| TESTI4008816 | 1578 | 661 . . . 3057 | 3766 |
| TESTI4008935 | 1579 | 2086 . . . 3030 | 3767 |
| TESTI4008993 | 1580 | 224 . . . 1048 | 3768 |
| TESTI4009022 | 1581 | 2341 . . . 2706 | 3769 |
| TESTI4009034 | 1582 | 4067 . . . 4510 | 3770 |
| TESTI4009123 | 1583 | 33 . . . 374 | 3771 |
| TESTI4009160 | 1584 | 51 . . . 2603 | 3772 |
| TESTI4009215 | 1585 | 1159 . . . 1467 | 3773 |
| TESTI4009283 | 1586 | 1816 . . . 2547 | 3774 |
| TESTI4009286 | 1587 | 4041 . . . 4886 | 3775 |
| TESTI4009374 | 1588 | 180 . . . 1592 | 3776 |
| TESTI4009406 | 1589 | 2151 . . . 3299 | 3777 |
| TESTI4009457 | 1590 | 364 . . . 858 | 3778 |
| TESTI4009563 | 1591 | 2555 . . . 3313 | 3779 |
| TESTI4009608 | 1592 | 531 . . . 908 | 3780 |
| TESTI4009638 | 1593 | 85 . . . 624 | 3781 |
| TESTI4009881 | 1594 | 583 . . . 3669 | 3782 |
| TESTI4010211 | 1595 | 46 . . . 405 | 3783 |
| TESTI4010377 | 1596 | 1057 . . . 1476 | 3784 |
| TESTI4010713 | 1597 | 106 . . . 3780 | 3785 |
| TESTI4010789 | 1598 | 2218 . . . 2778 | 3786 |
| TESTI4010817 | 1599 | 1062 . . . >3380 | 3787 |
| TESTI4010831 | 1600 | 486 . . . 3395 | 3788 |
| TESTI4010851 | 1601 | 92 . . . >4555 | 3789 |
| TESTI4010928 | 1602 | 197 . . . 769 | 3790 |
| TESTI4011118 | 1603 | 4088 . . . 4399 | 3791 |
| TESTI4011161 | 1604 | 1397 . . . 3091 | 3792 |
| TESTI4011246 | 1605 | 1723 . . . 2106 | 3793 |
| TESTI4011484 | 1606 | 201 . . . 2336 | 3794 |
| TESTI4011505 | 1607 | 1357 . . . 1815 | 3795 |
| TESTI4011745 | 1608 | 2319 . . . >4990 | 3796 |
| TESTI4011956 | 1609 | 162 . . . 2786 | 3797 |
| TESTI4012086 | 1610 | 177 . . . 731 | 3798 |
| TESTI4012329 | 1611 | 524 . . . 907 | 3799 |
| TESTI4012406 | 1612 | 141 . . . 611 | 3800 |
| TESTI4012448 | 1613 | 1666 . . . 3051 | 3801 |
| TESTI4012505 | 1614 | 1119 . . . 3005 | 3802 |
| TESTI4012556 | 1615 | 1928 . . . 2431 | 3803 |
| TESTI4012679 | 1616 | 1037 . . . 2071 | 3804 |
| TESTI4012702 | 1617 | 72 . . . 1529 | 3805 |
| TESTI4013369 | 1618 | 3026 . . . 3454 | 3806 |
| TESTI4013667 | 1619 | 351 . . . 659 | 3807 |
| TESTI4013675 | 1620 | 287 . . . 592 | 3808 |
| TESTI4013685 | 1621 | 2650 . . . 3045 | 3809 |
| TESTI4013735 | 1622 | 2964 . . . 3386 | 3810 |
| TESTI4013817 | 1623 | 2764 . . . 3237 | 3811 |
| TESTI4013830 | 1624 | 163 . . . 4938 | 3812 |
| TESTI4013924 | 1625 | 168 . . . 2039 | 3813 |
| TESTI4014159 | 1626 | 2462 . . . 2929 | 3814 |
| TESTI4014175 | 1627 | 836 . . . 2569 | 3815 |
| TESTI4014306 | 1628 | 2600 . . . 3031 | 3816 |
| TESTI4014392 | 1629 | 2506 . . . 2877 | 3817 |
| TESTI4014445 | 1630 | 23 . . . 364 | 3818 |
| TESTI4014694 | 1631 | 65 . . . 424 | 3819 |
| TESTI4014818 | 1632 | 1881 . . . 2957 | 3820 |
| TESTI4014924 | 1633 | 131 . . . 3898 | 3821 |
| TESTI4015263 | 1634 | 246 . . . 746 | 3822 |
| TESTI4015293 | 1635 | 2214 . . . 4253 | 3823 |
| TESTI4015471 | 1636 | 3317 . . . 3808 | 3824 |
| TESTI4015600 | 1637 | 2050 . . . 2508 | 3825 |
| TESTI4015646 | 1638 | 1674 . . . 2030 | 3826 |
| TESTI4015681 | 1639 | 2551 . . . 3006 | 3827 |
| TESTI4015688 | 1640 | 37 . . . 432 | 3828 |
| TESTI4016110 | 1641 | 2559 . . . 3257 | 3829 |
| TESTI4016238 | 1642 | 12 . . . 371 | 3830 |
| TESTI4016551 | 1643 | 2731 . . . 3495 | 3831 |
| TESTI4016812 | 1644 | 53 . . . 3097 | 3832 |
| TESTI4016822 | 1645 | 203 . . . 688 | 3833 |
| TESTI4016882 | 1646 | 131 . . . 958 | 3834 |
| TESTI4016925 | 1647 | 49 . . . 4704 | 3835 |
| TESTI4017001 | 1648 | 1 . . . 1251 | 3836 |
| TESTI4017137 | 1649 | 1822 . . . 2391 | 3837 |
| TESTI4017254 | 1650 | 295 . . . 645 | 3838 |
| TESTI4017543 | 1651 | 30 . . . 3824 | 3839 |
| TESTI4017575 | 1652 | 32 . . . 3679 | 3840 |
| TESTI4017848 | 1653 | 170 . . . 544 | 3841 |
| TESTI4017901 | 1654 | 50 . . . 361 | 3842 |
| TESTI4017961 | 1655 | 2224 . . . 2679 | 3843 |
| TESTI4018152 | 1656 | 1279 . . . 2082 | 3844 |
| TESTI4018208 | 1657 | 114 . . . 461 | 3845 |
| TESTI4018382 | 1658 | 819 . . . 1208 | 3846 |
| TESTI4018555 | 1659 | 980 . . . 1633 | 3847 |
| TESTI4018806 | 1660 | 156 . . . 587 | 3848 |
| TESTI4018835 | 1661 | 1223 . . . 3052 | 3849 |
| TESTI4018881 | 1662 | 1303 . . . 2754 | 3850 |
| TESTI4018886 | 1663 | 1235 . . . 2332 | 3851 |
| TESTI4019140 | 1664 | 622 . . . 2322 | 3852 |
| TESTI4019299 | 1665 | 122 . . . 1195 | 3853 |
| TESTI4019417 | 1666 | 247 . . . 618 | 3854 |
| TESTI4019566 | 1667 | 35 . . . 2755 | 3855 |
| TESTI4019843 | 1668 | 165 . . . 2381 | 3856 |
| TESTI4020092 | 1669 | 1074 . . . 1418 | 3857 |
| TESTI4020102 | 1670 | 1262 . . . 1693 | 3858 |
| TESTI4020806 | 1671 | 367 . . . 753 | 3859 |
| TESTI4020920 | 1672 | 846 . . . 2930 | 3860 |
| TESTI4021294 | 1673 | 1 . . . 3249 | 3861 |
| TESTI4021456 | 1674 | 445 . . . 900 | 3862 |
| TESTI4021478 | 1675 | 116 . . . 2476 | 3863 |
| TESTI4021491 | 1676 | 1532 . . . 1972 | 3864 |
| TESTI4022716 | 1677 | 577 . . . 3141 | 3865 |
| TESTI4022873 | 1678 | 166 . . . 3783 | 3866 |
| TESTI4022936 | 1679 | 54 . . . 854 | 3867 |
| TESTI4023546 | 1680 | 150 . . . 2882 | 3868 |
| TESTI4023555 | 1681 | 1769 . . . 2470 | 3869 |
| TESTI4023722 | 1682 | 729 . . . 1112 | 3870 |
| TESTI4023762 | 1683 | 471 . . . 2999 | 3871 |
| TESTI4023942 | 1684 | 47 . . . 361 | 3872 |
| TESTI4024344 | 1685 | 3 . . . 548 | 3873 |
| TESTI4024420 | 1686 | 96 . . . 3140 | 3874 |
| TESTI4024874 | 1687 | 418 . . . 918 | 3875 |
| TESTI4024890 | 1688 | 2189 . . . 2623 | 3876 |
| TESTI4024907 | 1689 | 1280 . . . 1729 | 3877 |
| TESTI4025731 | 1690 | 266 . . . 610 | 3878 |
| TESTI4025797 | 1691 | 56 . . . 2869 | 3879 |
| TESTI4025920 | 1692 | 76 . . . 3117 | 3880 |
| TESTI4026079 | 1693 | 3 . . . 323 | 3881 |
| TESTI4026192 | 1694 | 1 . . . 390 | 3882 |
| TESTI4026295 | 1695 | 145 . . . 498 | 3883 |
| TESTI4026456 | 1696 | 117 . . . 443 | 3884 |
| TESTI4026510 | 1697 | 1733 . . . 3232 | 3885 |
| TESTI4026524 | 1698 | 744 . . . 2759 | 3886 |
| TESTI4026700 | 1699 | 242 . . . 2626 | 3887 |
| TESTI4026762 | 1700 | 392 . . . 4093 | 3888 |
| TESTI4026785 | 1701 | 107 . . . 508 | 3889 |
| TESTI4027516 | 1702 | 3747 . . . 4265 | 3890 |
| TESTI4027557 | 1703 | 1469 . . . 2440 | 3891 |
| TESTI4027821 | 1704 | 1226 . . . 1855 | 3892 |
| TESTI4028059 | 1705 | 1786 . . . 3183 | 3893 |
| TESTI4028062 | 1706 | 53 . . . 529 | 3894 |
| TESTI4028429 | 1707 | 1 . . . 306 | 3895 |
| TESTI4028612 | 1708 | 357 . . . 4304 | 3896 |
| TESTI4028809 | 1709 | 1672 . . . 2172 | 3897 |
| TESTI4028823 | 1710 | 119 . . . >2529 | 3898 |
| TESTI4028880 | 1711 | 758 . . . 2320 | 3899 |
| TESTI4028983 | 1712 | 283 . . . 924 | 3900 |
| TESTI4029370 | 1713 | 79 . . . 447 | 3901 |
| TESTI4029671 | 1714 | 59 . . . 1408 | 3902 |
| TESTI4029836 | 1715 | 76 . . . 2982 | 3903 |
| TESTI4030069 | 1716 | 219 . . . 650 | 3904 |
| TESTI4030159 | 1717 | 34 . . . 621 | 3905 |
| TESTI4030505 | 1718 | 142 . . . 3492 | 3906 |
| TESTI4030603 | 1719 | 37 . . . 1011 | 3907 |
| TESTI4030669 | 1720 | 3 . . . 3041 | 3908 |
| TESTI4032895 | 1721 | 212 . . . 2701 | 3909 |
| TESTI4033433 | 1722 | 875 . . . 1519 | 3910 |
| TESTI4033690 | 1723 | 206 . . . >3057 | 3911 |
| TESTI4034172 | 1724 | 97 . . . 435 | 3912 |
| TESTI4034212 | 1725 | 296 . . . 1093 | 3913 |
| TESTI4034432 | 1726 | 4035 . . . >4449 | 3914 |
| TESTI4034632 | 1727 | 1306 . . . 2199 | 3915 |
| TESTI4034912 | 1728 | 201 . . . 3137 | 3916 |
| TESTI4035063 | 1729 | 894 . . . 3518 | 3917 |
| TESTI4035065 | 1730 | 1418 . . . 1927 | 3918 |
| TESTI4035498 | 1731 | 39 . . . 704 | 3919 |
| TESTI4035602 | 1732 | 39 . . . 389 | 3920 |
| TESTI4035637 | 1733 | 1 . . . 2937 | 3921 |
| TESTI4035649 | 1734 | 19 . . . 441 | 3922 |
| TESTI4036042 | 1735 | 3085 . . . 3465 | 3923 |
| TESTI4036909 | 1736 | 130 . . . 2910 | 3924 |
| TESTI4037066 | 1737 | 1285 . . . 4461 | 3925 |
| TESTI4037156 | 1738 | 1119 . . . 2738 | 3926 |
| TESTI4037188 | 1739 | 142 . . . 2280 | 3927 |
| TESTI4037244 | 1740 | 52 . . . 354 | 3928 |
| TESTI4037727 | 1741 | 250 . . . 3051 | 3929 |
| TESTI4038156 | 1742 | 163 . . . 489 | 3930 |
| TESTI4038223 | 1743 | 112 . . . 654 | 3931 |
| TESTI4038258 | 1744 | 83 . . . 403 | 3932 |
| TESTI4038339 | 1745 | 458 . . . 2428 | 3933 |
| TESTI4038492 | 1746 | 415 . . . 960 | 3934 |
| TESTI4038818 | 1747 | 1053 . . . 1442 | 3935 |
| TESTI4039038 | 1748 | 1270 . . . 2871 | 3936 |
| TESTI4039086 | 1749 | 1902 . . . 2606 | 3937 |
| TESTI4039659 | 1750 | 237 . . . 935 | 3938 |
| TESTI4040363 | 1751 | 255 . . . 677 | 3939 |
| TESTI4040800 | 1752 | 59 . . . 1144 | 3940 |
| TESTI4040939 | 1753 | 23 . . . 823 | 3941 |
| TESTI4040956 | 1754 | 1316 . . . 1630 | 3942 |
| TESTI4041053 | 1755 | 89 . . . 3331 | 3943 |
| TESTI4041099 | 1756 | 3010 . . . 3369 | 3944 |
| TESTI4041143 | 1757 | 318 . . . 644 | 3945 |
| TESTI4041519 | 1758 | 4 . . . 345 | 3946 |
| TESTI4041624 | 1759 | 6 . . . 458 | 3947 |
| TESTI4041903 | 1760 | 69 . . . 458 | 3948 |
| TESTI4041954 | 1761 | 130 . . . 498 | 3949 |
| TESTI4042098 | 1762 | 1523 . . . 2296 | 3950 |
| TESTI4042444 | 1763 | 1808 . . . 2239 | 3951 |
| TESTI4042711 | 1764 | 220 . . . 561 | 3952 |
| TESTI4043129 | 1765 | 1129 . . . 1629 | 3953 |
| TESTI4043203 | 1766 | 84 . . . 437 | 3954 |
| TESTI4043551 | 1767 | 2185 . . . 2754 | 3955 |
| TESTI4043947 | 1768 | 14 . . . 2920 | 3956 |
| TESTI4044035 | 1769 | 140 . . . 682 | 3957 |
| TESTI4044084 | 1770 | 6 . . . 491 | 3958 |
| TESTI4044123 | 1771 | 227 . . . 637 | 3959 |
| TESTI4044186 | 1772 | 263 . . . 844 | 3960 |
| TESTI4044234 | 1773 | 184 . . . 987 | 3961 |
| TESTI4044296 | 1774 | 1968 . . . 2684 | 3962 |
| TESTI4044682 | 1775 | 36 . . . 491 | 3963 |
| TESTI4045312 | 1776 | 375 . . . 734 | 3964 |
| TESTI4046253 | 1777 | 151 . . . 456 | 3965 |
| TESTI4046282 | 1778 | 79 . . . 3672 | 3966 |
| TESTI4046487 | 1779 | 1351 . . . 2883 | 3967 |
| TESTI4046819 | 1780 | 299 . . . 3535 | 3968 |
| TESTI4046884 | 1781 | 99 . . . >3063 | 3969 |
| TESTI4047069 | 1782 | 1554 . . . 1895 | 3970 |
| THYMU1000496 | 1783 | 324 . . . 1295 | 3971 |
| THYMU1000600 | 1784 | 390 . . . 809 | 3972 |
| THYMU2000932 | 1785 | 1767 . . . 2492 | 3973 |
| THYMU2001053 | 1786 | 1669 . . . 2070 | 3974 |
| THYMU2001090 | 1787 | 1738 . . . 2067 | 3975 |
| THYMU2003397 | 1788 | 1100 . . . 1543 | 3976 |
| THYMU2003632 | 1789 | 1483 . . . 1812 | 3977 |
| THYMU2003760 | 1790 | 304 . . . 612 | 3978 |
| THYMU2004693 | 1791 | 1445 . . . 1792 | 3979 |
| THYMU2005003 | 1792 | 1245 . . . 1697 | 3980 |
| THYMU2005190 | 1793 | 133 . . . 531 | 3981 |
| THYMU2005303 | 1794 | 909 . . . 1367 | 3982 |
| THYMU2005321 | 1795 | 81 . . . 473 | 3983 |
| THYMU2006420 | 1796 | 335 . . . 1147 | 3984 |
| THYMU2007060 | 1797 | 786 . . . >2283 | 3985 |
| THYMU2007179 | 1798 | 310 . . . >1233 | 3986 |
| THYMU2007658 | 1799 | 452 . . . 913 | 3987 |
| THYMU2008282 | 1800 | 1301 . . . 1741 | 3988 |
| THYMU2008725 | 1801 | 47 . . . 1876 | 3989 |
| THYMU2009134 | 1802 | 147 . . . 674 | 3990 |
| THYMU2009157 | 1803 | 1691 . . . 2095 | 3991 |
| THYMU2009425 | 1804 | 4 . . . 525 | 3992 |
| THYMU2011548 | 1805 | 70 . . . 1044 | 3993 |
| THYMU2011736 | 1806 | 1019 . . . 1939 | 3994 |
| THYMU2013386 | 1807 | 421 . . . 1773 | 3995 |
| THYMU2014353 | 1808 | 765 . . . 1100 | 3996 |
| THYMU2016204 | 1809 | 880 . . . 1923 | 3997 |
| THYMU2016523 | 1810 | 2076 . . . 2411 | 3998 |
| THYMU2019210 | 1811 | 55 . . . 1359 | 3999 |
| THYMU2019587 | 1812 | 1196 . . . 1633 | 4000 |
| THYMU2023711 | 1813 | 80 . . . 1486 | 4001 |
| THYMU2023967 | 1814 | 1000 . . . 1479 | 4002 |
| THYMU2025707 | 1815 | 18 . . . 527 | 4003 |
| THYMU2027497 | 1816 | 84 . . . 884 | 4004 |
| THYMU2027695 | 1817 | 52 . . . 1611 | 4005 |
| THYMU2027734 | 1818 | 581 . . . 1141 | 4006 |
| THYMU2028978 | 1819 | 283 . . . 642 | 4007 |
| THYMU2029676 | 1820 | 1209 . . . 1868 | 4008 |
| THYMU2029688 | 1821 | 45 . . . 368 | 4009 |
| THYMU2030068 | 1822 | 91 . . . 477 | 4010 |
| THYMU2030226 | 1823 | 207 . . . 710 | 4011 |
| THYMU2030264 | 1824 | 152 . . . 1756 | 4012 |
| THYMU2030637 | 1825 | 147 . . . 1865 | 4013 |
| THYMU2030796 | 1826 | 328 . . . 642 | 4014 |
| THYMU2031046 | 1827 | 23 . . . 703 | 4015 |
| THYMU2031218 | 1828 | 563 . . . 925 | 4016 |
| THYMU2031258 | 1829 | 127 . . . 681 | 4017 |
| THYMU2031341 | 1830 | 2050 . . . 2376 | 4018 |
| THYMU2031368 | 1831 | 498 . . . 821 | 4019 |
| THYMU2031579 | 1832 | 258 . . . 578 | 4020 |
| THYMU2031847 | 1833 | 145 . . . 492 | 4021 |
| THYMU2031890 | 1834 | 4 . . . 348 | 4022 |
| THYMU2032014 | 1835 | 880 . . . 1371 | 4023 |
| THYMU2032035 | 1836 | 186 . . . 533 | 4024 |
| THYMU2032080 | 1837 | 366 . . . 674 | 4025 |
| THYMU2032358 | 1838 | 230 . . . 604 | 4026 |
| THYMU2032437 | 1839 | 665 . . . 1090 | 4027 |
| THYMU2032655 | 1840 | 360 . . . 701 | 4028 |
| THYMU2032696 | 1841 | 1133 . . . 2215 | 4029 |
| THYMU2032825 | 1842 | 189 . . . 818 | 4030 |
| THYMU2033070 | 1843 | 22 . . . 441 | 4031 |
| THYMU2033079 | 1844 | 1706 . . . 2023 | 4032 |
| THYMU2033104 | 1845 | 1963 . . . 2298 | 4033 |
| THYMU2033308 | 1846 | 1067 . . . 1369 | 4034 |
| THYMU2033787 | 1847 | 1808 . . . 2656 | 4035 |
| THYMU2033816 | 1848 | 1947 . . . 2336 | 4036 |
| THYMU2034314 | 1849 | 1836 . . . 2189 | 4037 |
| THYMU2034374 | 1850 | 1119 . . . 1637 | 4038 |
| THYMU2034647 | 1851 | 1557 . . . 2051 | 4039 |
| THYMU2035064 | 1852 | 24 . . . 428 | 4040 |
| THYMU2035101 | 1853 | 17 . . . 334 | 4041 |
| THYMU2035319 | 1854 | 641 . . . 2161 | 4042 |
| THYMU2035388 | 1855 | 37 . . . 408 | 4043 |
| THYMU2035400 | 1856 | 96 . . . 494 | 4044 |
| THYMU2035735 | 1857 | 99 . . . 1619 | 4045 |
| THYMU2036058 | 1858 | 1381 . . . 1785 | 4046 |
| THYMU2036085 | 1859 | 2311 . . . 2922 | 4047 |
| THYMU2036252 | 1860 | 22 . . . 459 | 4048 |
| THYMU2036265 | 1861 | 1260 . . . 1568 | 4049 |
| THYMU2036459 | 1862 | 347 . . . 3121 | 4050 |
| THYMU2036653 | 1863 | 422 . . . 754 | 4051 |
| THYMU2037081 | 1864 | 78 . . . 533 | 4052 |
| THYMU2037208 | 1865 | 1156 . . . 1608 | 4053 |
| THYMU2037226 | 1866 | 375 . . . 4157 | 4054 |
| THYMU2037233 | 1867 | 2777 . . . 3235 | 4055 |
| THYMU2037348 | 1868 | 115 . . . 423 | 4056 |
| THYMU2037965 | 1869 | 167 . . . 598 | 4057 |
| THYMU2038189 | 1870 | 84 . . . 452 | 4058 |
| THYMU2038301 | 1871 | 1019 . . . 1417 | 4059 |
| THYMU2038369 | 1872 | 11 . . . 826 | 4060 |
| THYMU2038615 | 1873 | 597 . . . 1100 | 4061 |
| THYMU2038636 | 1874 | 19 . . . 387 | 4062 |
| THYMU2038739 | 1875 | 1446 . . . 1874 | 4063 |
| THYMU2038772 | 1876 | 50 . . . 532 | 4064 |
| THYMU2038797 | 1877 | 75 . . . 1304 | 4065 |
| THYMU2039305 | 1878 | 1607 . . . 1969 | 4066 |
| THYMU2039315 | 1879 | 26 . . . 1549 | 4067 |
| THYMU2039350 | 1880 | 86 . . . 1891 | 4068 |
| THYMU2039411 | 1881 | 423 . . . 800 | 4069 |
| THYMU2039780 | 1882 | 292 . . . 831 | 4070 |
| THYMU2039989 | 1883 | 1651 . . . 2124 | 4071 |
| THYMU2040140 | 1884 | 72 . . . 437 | 4072 |
| THYMU2040412 | 1885 | 660 . . . 1049 | 4073 |
| THYMU2040824 | 1886 | 13 . . . 693 | 4074 |
| THYMU2040975 | 1887 | 174 . . . 1262 | 4075 |
| THYMU2041007 | 1888 | 54 . . . 401 | 4076 |
| THYMU2041015 | 1889 | 251 . . . 1798 | 4077 |
| THYMU2041252 | 1890 | 301 . . . 798 | 4078 |
| THYMU3000028 | 1891 | 883 . . . 1701 | 4079 |
| THYMU3000036 | 1892 | 480 . . . 881 | 4080 |
| THYMU3000133 | 1893 | 312 . . . 2012 | 4081 |
| THYMU3000655 | 1894 | 148 . . . 774 | 4082 |
| THYMU3000826 | 1895 | 225 . . . 779 | 4083 |
| THYMU3001083 | 1896 | 2429 . . . 2761 | 4084 |
| THYMU3001234 | 1897 | 1340 . . . 2329 | 4085 |
| THYMU3001379 | 1898 | 599 . . . 2251 | 4086 |
| THYMU3001472 | 1899 | 1556 . . . 1870 | 4087 |
| THYMU3001991 | 1900 | 2453 . . . 2782 | 4088 |
| THYMU3002452 | 1901 | 1314 . . . 1697 | 4089 |
| THYMU3002661 | 1902 | 818 . . . 1348 | 4090 |
| THYMU3003212 | 1903 | 1292 . . . 1726 | 4091 |
| THYMU3003309 | 1904 | 2336 . . . 2866 | 4092 |
| THYMU3003763 | 1905 | 1011 . . . 1991 | 4093 |
| THYMU3004157 | 1906 | 579 . . . 899 | 4094 |
| THYMU3004835 | 1907 | 77 . . . 1282 | 4095 |
| THYMU3004866 | 1908 | 703 . . . 2709 | 4096 |
| THYMU3005696 | 1909 | 2267 . . . 2569 | 4097 |
| THYMU3006118 | 1910 | 924 . . . 1331 | 4098 |
| THYMU3006132 | 1911 | 237 . . . 1703 | 4099 |
| THYMU3006168 | 1912 | 2025 . . . 2813 | 4100 |
| THYMU3006172 | 1913 | 240 . . . 1823 | 4101 |
| THYMU3006371 | 1914 | 1600 . . . 2007 | 4102 |
| THYMU3006485 | 1915 | 1386 . . . 1796 | 4103 |
| THYMU3006811 | 1916 | 27 . . . 395 | 4104 |
| THYMU3006963 | 1917 | 386 . . . 784 | 4105 |
| THYMU3007137 | 1918 | 404 . . . 2878 | 4106 |
| THYMU3007368 | 1919 | 2076 . . . 2516 | 4107 |
| THYMU3007845 | 1920 | 1862 . . . 2281 | 4108 |
| THYMU3008171 | 1921 | 1204 . . . 1773 | 4109 |
| THYMU3008436 | 1922 | 225 . . . 2780 | 4110 |
| THYMU3009255 | 1923 | 186 . . . 524 | 4111 |
| TKIDN2000701 | 1924 | 1716 . . . 2306 | 4112 |
| TKIDN2002424 | 1925 | 42 . . . 377 | 4113 |
| TKIDN2002632 | 1926 | 350 . . . 730 | 4114 |
| TKIDN2003044 | 1927 | 1520 . . . 1840 | 4115 |
| TKIDN2004386 | 1928 | 1489 . . . 2130 | 4116 |
| TKIDN2005934 | 1929 | 158 . . . 571 | 4117 |
| TKIDN2005947 | 1930 | 144 . . . 533 | 4118 |
| TKIDN2006525 | 1931 | 613 . . . 1590 | 4119 |
| TKIDN2006852 | 1932 | 665 . . . 1294 | 4120 |
| TKIDN2007667 | 1933 | 1640 . . . >2145 | 4121 |
| TKIDN2009092 | 1934 | 665 . . . 1021 | 4122 |
| TKIDN2009641 | 1935 | 538 . . . 852 | 4123 |
| TKIDN2009889 | 1936 | 164 . . . 580 | 4124 |
| TKIDN2010934 | 1937 | 516 . . . 1220 | 4125 |
| TKIDN2012824 | 1938 | 434 . . . 745 | 4126 |
| TKIDN2013287 | 1939 | 331 . . . 663 | 4127 |
| TKIDN2014757 | 1940 | 223 . . . 537 | 4128 |
| TKIDN2014771 | 1941 | 244 . . . 642 | 4129 |
| TKIDN2015263 | 1942 | 43 . . . 396 | 4130 |
| TKIDN2015788 | 1943 | 841 . . . 1272 | 4131 |
| TKIDN2016309 | 1944 | 212 . . . 634 | 4132 |
| TKIDN2019116 | 1945 | 14 . . . 640 | 4133 |
| TLIVE2000023 | 1946 | 110 . . . 589 | 4134 |
| TLIVE2001327 | 1947 | 90 . . . 2918 | 4135 |
| TLIVE2001828 | 1948 | 155 . . . 589 | 4136 |
| TLIVE2001927 | 1949 | 557 . . . 895 | 4137 |
| TLIVE2002336 | 1950 | 3 . . . 1598 | 4138 |
| TLIVE2002338 | 1951 | 1005 . . . 1817 | 4139 |
| TLIVE2002690 | 1952 | 210 . . . 1490 | 4140 |
| TLIVE2003197 | 1953 | 88 . . . 474 | 4141 |
| TLIVE2003225 | 1954 | 60 . . . 1220 | 4142 |
| TLIVE2003381 | 1955 | 603 . . . 1097 | 4143 |
| TLIVE2003970 | 1956 | 230 . . . 640 | 4144 |
| TLIVE2004110 | 1957 | 68 . . . 424 | 4145 |
| TLIVE2004320 | 1958 | 1436 . . . 2650 | 4146 |
| TLIVE2004601 | 1959 | 242 . . . 586 | 4147 |
| TLIVE2005180 | 1960 | 1180 . . . 1518 | 4148 |
| TLIVE2006236 | 1961 | 59 . . . 409 | 4149 |
| TLIVE2006529 | 1962 | 572 . . . 1492 | 4150 |
| TLIVE2007132 | 1963 | 70 . . . 444 | 4151 |
| TLIVE2007528 | 1964 | 715 . . . 1110 | 4152 |
| TLIVE2007816 | 1965 | 214 . . . 669 | 4153 |
| TLIVE2008083 | 1966 | 1670 . . . 2002 | 4154 |
| TLIVE2008229 | 1967 | 17 . . . 1786 | 4155 |
| TLIVE2009541 | 1968 | 343 . . . 1413 | 4156 |
| TOVAR2000649 | 1969 | 192 . . . 605 | 4157 |
| TOVAR2001281 | 1970 | 71 . . . 403 | 4158 |
| TOVAR2001730 | 1971 | 94 . . . 672 | 4159 |
| TOVAR2002247 | 1972 | 317 . . . 1375 | 4160 |
| TOVAR2002549 | 1973 | 1301 . . . 1657 | 4161 |
| TRACH1000205 | 1974 | 89 . . . 928 | 4162 |
| TRACH2001443 | 1975 | 284 . . . 949 | 4163 |
| TRACH2001549 | 1976 | 190 . . . 1566 | 4164 |
| TRACH2001684 | 1977 | 1190 . . . 1684 | 4165 |
| TRACH2003070 | 1978 | 358 . . . 855 | 4166 |
| TRACH2004170 | 1979 | 349 . . . 795 | 4167 |
| TRACH2005066 | 1980 | 136 . . . 597 | 4168 |
| TRACH2005811 | 1981 | 1125 . . . 2234 | 4169 |
| TRACH2006049 | 1982 | 927 . . . 1247 | 4170 |
| TRACH2006387 | 1983 | 245 . . . 1258 | 4171 |
| TRACH2007059 | 1984 | 244 . . . 2031 | 4172 |
| TRACH2007834 | 1985 | 3 . . . 311 | 4173 |
| TRACH2008300 | 1986 | 414 . . . 752 | 4174 |
| TRACH2009310 | 1987 | 275 . . . 2413 | 4175 |
| TRACH2019248 | 1988 | 42 . . . 395 | 4176 |
| TRACH2019473 | 1989 | 179 . . . 1552 | 4177 |
| TRACH2020525 | 1990 | 1340 . . . 1912 | 4178 |
| TRACH2021398 | 1991 | 225 . . . 872 | 4179 |
| TRACH2021964 | 1992 | 105 . . . >2454 | 4180 |
| TRACH2022042 | 1993 | 985 . . . 1332 | 4181 |
| TRACH2022425 | 1994 | 80 . . . 1573 | 4182 |
| TRACH2022553 | 1995 | 38 . . . 1744 | 4183 |
| TRACH2022649 | 1996 | 55 . . . 1491 | 4184 |
| TRACH2023299 | 1997 | 24 . . . 1670 | 4185 |
| TRACH2023306 | 1998 | 157 . . . 1071 | 4186 |
| TRACH2025344 | 1999 | 1616 . . . 2086 | 4187 |
| TRACH2025507 | 2000 | 1483 . . . 1848 | 4188 |
| TRACH2025535 | 2001 | 561 . . . 1253 | 4189 |
| TRACH2025749 | 2002 | 655 . . . 1017 | 4190 |
| TRACH2025911 | 2003 | 189 . . . 680 | 4191 |
| TRACH2025932 | 2004 | 201 . . . 530 | 4192 |
| TRACH3000014 | 2005 | 33 . . . 2963 | 4193 |
| TRACH3000342 | 2006 | 3259 . . . 3672 | 4194 |
| TRACH3000558 | 2007 | 3058 . . . >3576 | 4195 |
| TRACH3000586 | 2008 | 1117 . . . 1662 | 4196 |
| TRACH3000926 | 2009 | 127 . . . 4203 | 4197 |
| TRACH3001427 | 2010 | 26 . . . 1021 | 4198 |
| TRACH3002064 | 2011 | 1457 . . . 1777 | 4199 |
| TRACH3002168 | 2012 | 730 . . . 2313 | 4200 |
| TRACH3002192 | 2013 | 24 . . . 464 | 4201 |
| TRACH3002650 | 2014 | 3122 . . . 3475 | 4202 |
| TRACH3002866 | 2015 | 90 . . . 1199 | 4203 |
| TRACH3002871 | 2016 | 1866 . . . 2183 | 4204 |
| TRACH3003379 | 2017 | 84 . . . 1916 | 4205 |
| TRACH3004068 | 2018 | 787 . . . 1104 | 4206 |
| TRACH3004537 | 2019 | 1535 . . . 2935 | 4207 |
| TRACH3004721 | 2020 | 1009 . . . 2847 | 4208 |
| TRACH3004786 | 2021 | 2629 . . . 3258 | 4209 |
| TRACH3004840 | 2022 | 79 . . . 714 | 4210 |
| TRACH3005294 | 2023 | 3984 . . . 4352 | 4211 |
| TRACH3005479 | 2024 | 90 . . . 3338 | 4212 |
| TRACH3005549 | 2025 | 81 . . . 845 | 4213 |
| TRACH3006038 | 2026 | 2846 . . . 3157 | 4214 |
| TRACH3006149 | 2027 | 2186 . . . 2494 | 4215 |
| TRACH3006228 | 2028 | 1016 . . . 3304 | 4216 |
| TRACH3006412 | 2029 | 1681 . . . 2196 | 4217 |
| TRACH3006470 | 2030 | 157 . . . 1983 | 4218 |
| TRACH3006889 | 2031 | 2712 . . . 3470 | 4219 |
| TRACH3007391 | 2032 | 3 . . . 308 | 4220 |
| TRACH3007479 | 2033 | 571 . . . 1602 | 4221 |
| TRACH3008093 | 2034 | 184 . . . 813 | 4222 |
| TRACH3008535 | 2035 | 2148 . . . 2456 | 4223 |
| TRACH3008629 | 2036 | 80 . . . 2737 | 4224 |
| TRACH3008713 | 2037 | 2044 . . . 2364 | 4225 |
| TRACH3009455 | 2038 | 1507 . . . 2718 | 4226 |
| TRACH3034731 | 2039 | 124 . . . 1233 | 4227 |
| TRACH3034762 | 2040 | 1177 . . . 1527 | 4228 |
| TRACH3035199 | 2041 | 234 . . . 1211 | 4229 |
| TRACH3035235 | 2042 | 3 . . . 350 | 4230 |
| TRACH3035482 | 2043 | 1879 . . . 2346 | 4231 |
| TRACH3035526 | 2044 | 17 . . . 1489 | 4232 |
| TRACH3036193 | 2045 | 172 . . . 4803 | 4233 |
| TRACH3036207 | 2046 | 268 . . . 1368 | 4234 |
| TRACH3036309 | 2047 | 50 . . . 691 | 4235 |
| TRACH3036456 | 2048 | 1214 . . . 1591 | 4236 |
| TRACH3036609 | 2049 | 3159 . . . 4154 | 4237 |
| TSTOM1000135 | 2050 | 201 . . . 1097 | 4238 |
| TSTOM2000442 | 2051 | 80 . . . 1633 | 4239 |
| TSTOM2000553 | 2052 | 170 . . . 1450 | 4240 |
| TSTOM2002672 | 2053 | 1088 . . . 1432 | 4241 |
| TUTER1000122 | 2054 | 10 . . . 363 | 4242 |
| TUTER2000425 | 2055 | 216 . . . 572 | 4243 |
| TUTER2000904 | 2056 | 18 . . . 773 | 4244 |
| TUTER2000916 | 2057 | 149 . . . 568 | 4245 |
| TUTER2001387 | 2058 | 1525 . . . 1908 | 4246 |
| TUTER2002729 | 2059 | 20 . . . 868 | 4247 |
| UTERU1000024 | 2060 | 15 . . . 1190 | 4248 |
| UTERU1000031 | 2061 | 86 . . . 1609 | 4249 |
| UTERU1000148 | 2062 | 1553 . . . 2005 | 4250 |
| UTERU1000249 | 2063 | 349 . . . 2499 | 4251 |
| UTERU1000337 | 2064 | 1062 . . . 1922 | 4252 |
| UTERU1000339 | 2065 | 18 . . . 341 | 4253 |
| UTERU2000649 | 2066 | 213 . . . 902 | 4254 |
| UTERU2001409 | 2067 | 236 . . . 553 | 4255 |
| UTERU2002410 | 2068 | 2655 . . . 2987 | 4256 |
| UTERU2002841 | 2069 | 1185 . . . 2021 | 4257 |
| UTERU2004688 | 2070 | 1487 . . . 2470 | 4258 |
| UTERU2004929 | 2071 | 2106 . . . 2462 | 4259 |
| UTERU2005004 | 2072 | 1795 . . . 2388 | 4260 |
| UTERU2005621 | 2073 | 98 . . . 1483 | 4261 |
| UTERU2006115 | 2074 | 128 . . . 1018 | 4262 |
| UTERU2006137 | 2075 | 1721 . . . 2068 | 4263 |
| UTERU2006568 | 2076 | 535 . . . 1458 | 4264 |
| UTERU2007444 | 2077 | 1104 . . . 1502 | 4265 |
| UTERU2007520 | 2078 | 1644 . . . 2126 | 4266 |
| UTERU2007724 | 2079 | 53 . . . 865 | 4267 |
| UTERU2008347 | 2080 | 123 . . . 1355 | 4268 |
| UTERU2014678 | 2081 | 1232 . . . 1585 | 4269 |
| UTERU2017762 | 2082 | 427 . . . 2319 | 4270 |
| UTERU2019491 | 2083 | 379 . . . 849 | 4271 |
| UTERU2019681 | 2084 | 1424 . . . 1840 | 4272 |
| UTERU2019706 | 2085 | 88 . . . 1656 | 4273 |
| UTERU2019940 | 2086 | 653 . . . 1228 | 4274 |
| UTERU2020491 | 2087 | 2042 . . . 2437 | 4275 |
| UTERU2020718 | 2088 | 355 . . . 1428 | 4276 |
| UTERU2021163 | 2089 | 1279 . . . 1620 | 4277 |
| UTERU2021380 | 2090 | 358 . . . 666 | 4278 |
| UTERU2022020 | 2091 | 1448 . . . 1789 | 4279 |
| UTERU2022981 | 2092 | 263 . . . 571 | 4280 |
| UTERU2023039 | 2093 | 172 . . . 681 | 4281 |
| UTERU2023175 | 2094 | 1231 . . . 2088 | 4282 |
| UTERU2023651 | 2095 | 647 . . . 1012 | 4283 |
| UTERU2023712 | 2096 | 399 . . . 758 | 4284 |
| UTERU2024002 | 2097 | 517 . . . 1026 | 4285 |
| UTERU2024656 | 2098 | 28 . . . 534 | 4286 |
| UTERU2025025 | 2099 | 814 . . . 2301 | 4287 |
| UTERU2025645 | 2100 | 263 . . . 1021 | 4288 |
| UTERU2025891 | 2101 | 1446 . . . 1787 | 4289 |
| UTERU2026025 | 2102 | 172 . . . 540 | 4290 |
| UTERU2026090 | 2103 | 65 . . . 1183 | 4291 |
| UTERU2026203 | 2104 | 612 . . . 1304 | 4292 |
| UTERU2027591 | 2105 | 427 . . . 747 | 4293 |
| UTERU2029953 | 2106 | 1415 . . . 1870 | 4294 |
| UTERU2030213 | 2107 | 1261 . . . 1950 | 4295 |
| UTERU2030280 | 2108 | 1134 . . . 1511 | 4296 |
| UTERU2031084 | 2109 | 162 . . . 728 | 4297 |
| UTERU2031268 | 2110 | 429 . . . 1985 | 4298 |
| UTERU2031521 | 2111 | 209 . . . 700 | 4299 |
| UTERU2031703 | 2112 | 1190 . . . 1654 | 4300 |
| UTERU2031851 | 2113 | 1192 . . . 1659 | 4301 |
| UTERU2033375 | 2114 | 887 . . . 2218 | 4302 |
| UTERU2033382 | 2115 | 1144 . . . 1614 | 4303 |
| UTERU2035114 | 2116 | 96 . . . 614 | 4304 |
| UTERU2035323 | 2117 | 1015 . . . 1467 | 4305 |
| UTERU2035328 | 2118 | 28 . . . >2438 | 4306 |
| UTERU2035331 | 2119 | 656 . . . 1255 | 4307 |
| UTERU2035452 | 2120 | 1393 . . . 1899 | 4308 |
| UTERU2035469 | 2121 | 261 . . . 707 | 4309 |
| UTERU2035503 | 2122 | 1539 . . . 1859 | 4310 |
| UTERU2035745 | 2123 | 1964 . . . 2272 | 4311 |
| UTERU2036089 | 2124 | 1131 . . . 2297 | 4312 |
| UTERU2037361 | 2125 | 1427 . . . 1849 | 4313 |
| UTERU2037577 | 2126 | 384 . . . 725 | 4314 |
| UTERU2038251 | 2127 | 76 . . . 1215 | 4315 |
| UTERU3000226 | 2128 | 1353 . . . 1691 | 4316 |
| UTERU3000645 | 2129 | 2681 . . . 3310 | 4317 |
| UTERU3000665 | 2130 | 1945 . . . 3624 | 4318 |
| UTERU3000828 | 2131 | 98 . . . 2986 | 4319 |
| UTERU3000899 | 2132 | 26 . . . 871 | 4320 |
| UTERU3001059 | 2133 | 2772 . . . 4715 | 4321 |
| UTERU3001240 | 2134 | 762 . . . 2021 | 4322 |
| UTERU3001542 | 2135 | 2792 . . . 3097 | 4323 |
| UTERU3001571 | 2136 | 2792 . . . 3466 | 4324 |
| UTERU3001572 | 2137 | 412 . . . 3990 | 4325 |
| UTERU3001585 | 2138 | 40 . . . 1551 | 4326 |
| UTERU3001652 | 2139 | 115 . . . 1590 | 4327 |
| UTERU3001766 | 2140 | 2128 . . . 2478 | 4328 |
| UTERU3001988 | 2141 | 28 . . . 801 | 4329 |
| UTERU3002209 | 2142 | 2047 . . . 2406 | 4330 |
| UTERU3002218 | 2143 | 221 . . . 3025 | 4331 |
| UTERU3002383 | 2144 | 248 . . . 571 | 4332 |
| UTERU3002667 | 2145 | 3011 . . . 3322 | 4333 |
| UTERU3002731 | 2146 | 161 . . . 472 | 4334 |
| UTERU3002768 | 2147 | 98 . . . 445 | 4335 |
| UTERU3002786 | 2148 | 1292 . . . 2065 | 4336 |
| UTERU3002993 | 2149 | 4072 . . . 4425 | 4337 |
| UTERU3003116 | 2150 | 3428 . . . 4126 | 4338 |
| UTERU3003135 | 2151 | 3370 . . . 3795 | 4339 |
| UTERU3003178 | 2152 | 1019 . . . 2077 | 4340 |
| UTERU3003465 | 2153 | 2851 . . . 3189 | 4341 |
| UTERU3003523 | 2154 | 176 . . . 3937 | 4342 |
| UTERU3003776 | 2155 | 711 . . . 1025 | 4343 |
| UTERU3004523 | 2156 | 1109 . . . 3427 | 4344 |
| UTERU3004616 | 2157 | 2480 . . . 3187 | 4345 |
| UTERU3004709 | 2158 | 718 . . . 1095 | 4346 |
| UTERU3004992 | 2159 | 2565 . . . 3077 | 4347 |
| UTERU3005049 | 2160 | 1561 . . . 2505 | 4348 |
| UTERU3005205 | 2161 | 101 . . . 1342 | 4349 |
| UTERU3005230 | 2162 | 1038 . . . 1445 | 4350 |
| UTERU3005460 | 2163 | 1872 . . . 2489 | 4351 |
| UTERU3005585 | 2164 | 1145 . . . 2395 | 4352 |
| UTERU3005907 | 2165 | 819 . . . 1709 | 4353 |
| UTERU3005970 | 2166 | 1 . . . 393 | 4354 |
| UTERU3006008 | 2167 | 2595 . . . 3236 | 4355 |
| UTERU3006308 | 2168 | 1490 . . . 2491 | 4356 |
| UTERU3007134 | 2169 | 2990 . . . 3532 | 4357 |
| UTERU3007419 | 2170 | 124 . . . 3792 | 4358 |
| UTERU3007640 | 2171 | 2823 . . . 3248 | 4359 |
| UTERU3007913 | 2172 | 138 . . . 1775 | 4360 |
| UTERU3008660 | 2173 | 3524 . . . 4030 | 4361 |
| UTERU3008671 | 2174 | 1920 . . . 2246 | 4362 |
| UTERU3009259 | 2175 | 2426 . . . 2917 | 4363 |
| UTERU3009490 | 2176 | 3200 . . . 3556 | 4364 |
| UTERU3009517 | 2177 | 2118 . . . 2786 | 4365 |
| UTERU3009690 | 2178 | 2674 . . . 2991 | 4366 |
| UTERU3009871 | 2179 | 111 . . . 1877 | 4367 |
| UTERU3009979 | 2180 | 1630 . . . 3504 | 4368 |
| UTERU3011063 | 2181 | 28 . . . 1614 | 4369 |
| UTERU3015086 | 2182 | 3216 . . . 3713 | 4370 |
| UTERU3015500 | 2183 | 985 . . . 2088 | 4371 |
| UTERU3016789 | 2184 | 553 . . . 1956 | 4372 |
| UTERU3018081 | 2185 | 180 . . . 3194 | 4373 |
| UTERU3018154 | 2186 | 1534 . . . 3186 | 4374 |
| UTERU3018616 | 2187 | 267 . . . 710 | 4375 |
| UTERU3018711 | 2188 | 3597 . . . 4106 | 4376 |
| 3NB692004724 | 4377 | 8 . . . 1486 | 4684 |
| ADRGL2000042 | 4378 | 133 . . . 996 | 4685 |
| ADRGL2000056 | 4379 | 158 . . . 748 | 4686 |
| BLADE2000579 | 4380 | 816 . . . 1703 | 4687 |
| BLADE2006830 | 4381 | 88 . . . 1872 | 4688 |
| BRACE2002589 | 4382 | 972 . . . 1334 | 4689 |
| BRACE2003609 | 4383 | 510 . . . 1895 | 4690 |
| BRACE2009318 | 4384 | 874 . . . 1242 | 4691 |
| BRACE2011677 | 4385 | 79 . . . 450 | 4692 |
| BRACE2029396 | 4386 | 38 . . . 421 | 4693 |
| BRACE2037299 | 4387 | 1170 . . . 1754 | 4694 |
| BRACE2039823 | 4388 | 153 . . . 866 | 4695 |
| BRACE2039832 | 4389 | 125 . . . 460 | 4696 |
| BRACE2043105 | 4390 | 96 . . . 494 | 4697 |
| BRACE3001058 | 4391 | 915 . . . 2912 | 4698 |
| BRACE3001113 | 4392 | 236 . . . 3196 | 4699 |
| BRACE3003026 | 4393 | 128 . . . 1453 | 4700 |
| BRACE3003053 | 4394 | 44 . . . >4212 | 4701 |
| BRACE3005107 | 4395 | 54 . . . 458 | 4702 |
| BRACE3009127 | 4396 | 151 . . . 2382 | 4703 |
| BRACE3010076 | 4397 | 1095 . . . 2828 | 4704 |
| BRACE3015829 | 4398 | 220 . . . 585 | 4705 |
| BRACE3021148 | 4399 | 29 . . . 637 | 4706 |
| BRALZ2017844 | 4400 | 173 . . . 1192 | 4707 |
| BRAMY2019111 | 4401 | 989 . . . 1768 | 4708 |
| BRAMY2035070 | 4402 | 287 . . . 2455 | 4709 |
| BRAMY2035449 | 4403 | 150 . . . 1418 | 4710 |
| BRAMY2035718 | 4404 | 353 . . . 1693 | 4711 |
| BRAMY2038516 | 4405 | 369 . . . 1835 | 4712 |
| BRAMY2039341 | 4406 | 127 . . . 1083 | 4713 |
| BRAMY2040159 | 4407 | 209 . . . 2440 | 4714 |
| BRAMY2041434 | 4408 | 125 . . . 550 | 4715 |
| BRAMY2045471 | 4409 | 148 . . . 2295 | 4716 |
| BRAMY3004800 | 4410 | 181 . . . 2736 | 4717 |
| BRAWH1000369 | 4411 | 412 . . . 1350 | 4718 |
| BRAWH2006207 | 4412 | 88 . . . 459 | 4719 |
| BRAWH2006395 | 4413 | 70 . . . 975 | 4720 |
| BRAWH2008993 | 4414 | 756 . . . 1061 | 4721 |
| BRAWH2009393 | 4415 | 179 . . . 529 | 4722 |
| BRAWH2010552 | 4416 | 72 . . . 572 | 4723 |
| BRAWH3007441 | 4417 | 2034 . . . 2531 | 4724 |
| BRAWH3009017 | 4418 | 733 . . . 1158 | 4725 |
| BRCAN2002473 | 4419 | 80 . . . 1060 | 4726 |
| BRCAN2002854 | 4420 | 36 . . . 842 | 4727 |
| BRCAN2003070 | 4421 | 1937 . . . 2254 | 4728 |
| BRCAN2014229 | 4422 | 249 . . . 1259 | 4729 |
| BRCOC2019841 | 4423 | 275 . . . 1591 | 4730 |
| BRHIP2002722 | 4424 | 411 . . . 2234 | 4731 |
| BRHIP2003272 | 4425 | 18 . . . 464 | 4732 |
| BRHIP2005271 | 4426 | 108 . . . 1286 | 4733 |
| BRHIP2005724 | 4427 | 107 . . . 1180 | 4734 |
| BRHIP2006617 | 4428 | 1568 . . . 2641 | 4735 |
| BRHIP2008389 | 4429 | 128 . . . 781 | 4736 |
| BRHIP2012360 | 4430 | 74 . . . 2800 | 4737 |
| BRHIP2017553 | 4431 | 362 . . . 2275 | 4738 |
| BRHIP2026877 | 4432 | 430 . . . 942 | 4739 |
| BRHIP3000017 | 4433 | 37 . . . 1515 | 4740 |
| BRHIP3000240 | 4434 | 290 . . . 1537 | 4741 |
| BRHIP3008314 | 4435 | 1622 . . . 2005 | 4742 |
| BRHIP3026052 | 4436 | 10 . . . 1632 | 4743 |
| BRSTN2013354 | 4437 | 286 . . . 1371 | 4744 |
| BRTHA2002133 | 4438 | 1448 . . . 1957 | 4745 |
| BRTHA2002702 | 4439 | 269 . . . 817 | 4746 |
| BRTHA2007060 | 4440 | 106 . . . 2979 | 4747 |
| BRTHA2010033 | 4441 | 823 . . . 1356 | 4748 |
| BRTHA2011321 | 4442 | 32 . . . 373 | 4749 |
| BRTHA2013426 | 4443 | 980 . . . 1588 | 4750 |
| BRTHA2013610 | 4444 | 71 . . . 1609 | 4751 |
| BRTHA2016318 | 4445 | 748 . . . 1581 | 4752 |
| BRTHA2017364 | 4446 | 52 . . . 1638 | 4753 |
| BRTHA2017972 | 4447 | 92 . . . 808 | 4754 |
| BRTHA2018011 | 4448 | 6 . . . 929 | 4755 |
| BRTHA2018443 | 4449 | 23 . . . 1774 | 4756 |
| BRTHA3000296 | 4450 | 2006 . . . 2521 | 4757 |
| BRTHA3003000 | 4451 | 1170 . . . 2510 | 4758 |
| BRTHA3008826 | 4452 | 2082 . . . 2402 | 4759 |
| CERVX2002013 | 4453 | 978 . . . 1301 | 4760 |
| CTONG1000113 | 4454 | 538 . . . 2541 | 4761 |
| CTONG2003348 | 4455 | 363 . . . 1745 | 4762 |
| CTONG2004000 | 4456 | 538 . . . 1869 | 4763 |
| CTONG2008721 | 4457 | 797 . . . 1909 | 4764 |
| CTONG2015596 | 4458 | 351 . . . 1490 | 4765 |
| CTONG2015633 | 4459 | 838 . . . 1305 | 4766 |
| CTONG2016942 | 4460 | 122 . . . 1387 | 4767 |
| CTONG2019822 | 4461 | 194 . . . 1681 | 4768 |
| CTONG2020374 | 4462 | 191 . . . 3052 | 4769 |
| CTONG2020378 | 4463 | 1693 . . . 2688 | 4770 |
| CTONG2020411 | 4464 | 23 . . . 3322 | 4771 |
| CTONG2020974 | 4465 | 968 . . . 1711 | 4772 |
| CTONG2024031 | 4466 | 256 . . . 2373 | 4773 |
| CTONG2028758 | 4467 | 268 . . . >2988 | 4774 |
| CTONG3001501 | 4468 | 233 . . . 1471 | 4775 |
| CTONG3002552 | 4469 | 3007 . . . >3950 | 4776 |
| CTONG3003598 | 4470 | 2111 . . . 2905 | 4777 |
| CTONG3004550 | 4471 | 310 . . . 2868 | 4778 |
| CTONG3004726 | 4472 | 84 . . . 2681 | 4779 |
| CTONG3009287 | 4473 | 745 . . . 2388 | 4780 |
| DFNES2011192 | 4474 | 209 . . . 1540 | 4781 |
| FCBBF1000509 | 4475 | 407 . . . 2356 | 4782 |
| FCBBF3010361 | 4476 | 253 . . . 1266 | 4783 |
| FCBBF3027854 | 4477 | 435 . . . >2218 | 4784 |
| FEBRA2000790 | 4478 | 483 . . . 839 | 4785 |
| FEBRA2001990 | 4479 | 57 . . . 1532 | 4786 |
| FEBRA2006519 | 4480 | 1794 . . . 3041 | 4787 |
| FEBRA2008692 | 4481 | 170 . . . 3094 | 4788 |
| FEBRA2014122 | 4482 | 216 . . . 2600 | 4789 |
| FEBRA2027609 | 4483 | 91 . . . 2169 | 4790 |
| FEBRA2028256 | 4484 | 365 . . . 2983 | 4791 |
| FEBRA2028516 | 4485 | 62 . . . 3109 | 4792 |
| HCASM2002754 | 4486 | 171 . . . 1508 | 4793 |
| HCASM2003018 | 4487 | 115 . . . >2321 | 4794 |
| HCASM2003099 | 4488 | 383 . . . 2119 | 4795 |
| HCASM2003357 | 4489 | 165 . . . 491 | 4796 |
| HCASM2008536 | 4490 | 201 . . . 629 | 4797 |
| HCASM2009424 | 4491 | 121 . . . 582 | 4798 |
| HCHON2000508 | 4492 | 59 . . . 2422 | 4799 |
| HCHON2000743 | 4493 | 218 . . . 883 | 4800 |
| HCHON2004858 | 4494 | 154 . . . 3285 | 4801 |
| HEART2009680 | 4495 | 63 . . . 1331 | 4802 |
| HLUNG2013350 | 4496 | 367 . . . 1299 | 4803 |
| HLUNG2015418 | 4497 | 73 . . . 2691 | 4804 |
| HLUNG2015548 | 4498 | 6 . . . 1598 | 4805 |
| HLUNG2016862 | 4499 | 295 . . . 699 | 4806 |
| HSYRA2005628 | 4500 | 454 . . . 1971 | 4807 |
| IMR322001879 | 4501 | 147 . . . 467 | 4808 |
| IMR322007078 | 4502 | 747 . . . 1979 | 4809 |
| IMR322008651 | 4503 | 145 . . . 1167 | 4810 |
| IMR322013396 | 4504 | 142 . . . 2238 | 4811 |
| IMR322013731 | 4505 | 81 . . . >1713 | 4812 |
| LIVER2000247 | 4506 | 1187 . . . 2191 | 4813 |
| MESAN2001770 | 4507 | 415 . . . 1770 | 4814 |
| MESAN2005303 | 4508 | 278 . . . 2362 | 4815 |
| MESAN2014412 | 4509 | 1589 . . . 3538 | 4816 |
| MESAN2015501 | 4510 | 553 . . . 2979 | 4817 |
| NT2RI2005772 | 4511 | 227 . . . 2035 | 4818 |
| NT2RI2008952 | 4512 | 187 . . . 1683 | 4819 |
| NT2RI2009583 | 4513 | 1127 . . . 2587 | 4820 |
| NT2RI2018448 | 4514 | 125 . . . 1030 | 4821 |
| NT2RI2027157 | 4515 | 603 . . . 2483 | 4822 |
| NT2RI3000174 | 4516 | 18 . . . 2501 | 4823 |
| NT2RI3001132 | 4517 | 281 . . . 3265 | 4824 |
| NT2RI3002557 | 4518 | 3660 . . . >3975 | 4825 |
| NT2RI3005928 | 4519 | 1784 . . . 2194 | 4826 |
| NT2RI3007167 | 4520 | 205 . . . 957 | 4827 |
| NT2RI3007443 | 4521 | 1024 . . . 3270 | 4828 |
| NT2RP7008435 | 4522 | 719 . . . 2437 | 4829 |
| NT2RP8000521 | 4523 | 635 . . . 1039 | 4830 |
| NTONG2008093 | 4524 | 81 . . . 635 | 4831 |
| OCBBF2003327 | 4525 | 35 . . . 1516 | 4832 |
| OCBBF2005433 | 4526 | 154 . . . 2565 | 4833 |
| OCBBF2006987 | 4527 | 447 . . . 3125 | 4834 |
| OCBBF2008144 | 4528 | 375 . . . >3049 | 4835 |
| OCBBF2009583 | 4529 | 443 . . . 1567 | 4836 |
| OCBBF2011669 | 4530 | 656 . . . 3346 | 4837 |
| OCBBF2019684 | 4531 | 161 . . . 1555 | 4838 |
| OCBBF2020048 | 4532 | 109 . . . 1152 | 4839 |
| OCBBF2024284 | 4533 | 70 . . . 3063 | 4840 |
| OCBBF2030116 | 4534 | 445 . . . 2574 | 4841 |
| OCBBF2032274 | 4535 | 46 . . . 1053 | 4842 |
| OCBBF2034637 | 4536 | 380 . . . 1897 | 4843 |
| OCBBF3000167 | 4537 | 139 . . . 1365 | 4844 |
| OCBBF3002654 | 4538 | 494 . . . 2284 | 4845 |
| OCBBF3003761 | 4539 | 316 . . . 2025 | 4846 |
| OCBBF3004972 | 4540 | 2394 . . . 3302 | 4847 |
| PERIC2007068 | 4541 | 174 . . . 1523 | 4848 |
| PLACE7000333 | 4542 | 1736 . . . 2470 | 4849 |
| PLACE7000502 | 4543 | 2490 . . . 4577 | 4850 |
| PROST2000452 | 4544 | 1462 . . . 2097 | 4851 |
| PROST2009320 | 4545 | 1640 . . . 2221 | 4852 |
| PROST2019487 | 4546 | 745 . . . 1149 | 4853 |
| PUAEN2006335 | 4547 | 123 . . . 1829 | 4854 |
| SKMUS2003194 | 4548 | 70 . . . 1317 | 4855 |
| SPLEN2004611 | 4549 | 1058 . . . 1651 | 4856 |
| SPLEN2016135 | 4550 | 70 . . . 699 | 4857 |
| SPLEN2016781 | 4551 | 1665 . . . 2444 | 4858 |
| SPLEN2016932 | 4552 | 597 . . . 1079 | 4859 |
| SPLEN2030847 | 4553 | 101 . . . 931 | 4860 |
| SPLEN2033490 | 4554 | 1418 . . . 1897 | 4861 |
| SPLEN2036702 | 4555 | 122 . . . 2467 | 4862 |
| SPLEN2037319 | 4556 | 20 . . . 364 | 4863 |
| SPLEN2039311 | 4557 | 343 . . . 768 | 4864 |
| SPLEN2039379 | 4558 | 4 . . . 2199 | 4865 |
| STOMA2003158 | 4559 | 593 . . . 1459 | 4866 |
| STOMA2004893 | 4560 | 1137 . . . >1566 | 4867 |
| SYNOV1000256 | 4561 | 2082 . . . 2927 | 4868 |
| SYNOV2001660 | 4562 | 917 . . . 1510 | 4869 |
| SYNOV2006620 | 4563 | 1036 . . . 1692 | 4870 |
| SYNOV2013637 | 4564 | 36 . . . 1085 | 4871 |
| SYNOV2021953 | 4565 | 375 . . . 1640 | 4872 |
| SYNOV4002744 | 4566 | 469 . . . 1302 | 4873 |
| SYNOV4003981 | 4567 | 36 . . . 2837 | 4874 |
| SYNOV4005739 | 4568 | 1425 . . . 2075 | 4875 |
| SYNOV4005889 | 4569 | 79 . . . 2793 | 4876 |
| TBAES2000932 | 4570 | 1943 . . . 2437 | 4877 |
| TESOP2000390 | 4571 | 218 . . . 1651 | 4878 |
| TESOP2001796 | 4572 | 129 . . . 1589 | 4879 |
| TESOP2005199 | 4573 | 108 . . . 1586 | 4880 |
| TESOP2006398 | 4574 | 2557 . . . >3253 | 4881 |
| TESOP2006865 | 4575 | 256 . . . 732 | 4882 |
| TESOP2007384 | 4576 | 168 . . . 1058 | 4883 |
| TESTI1000266 | 4577 | 346 . . . 807 | 4884 |
| TESTI2008901 | 4578 | 432 . . . 2120 | 4885 |
| TESTI2015626 | 4579 | 448 . . . 1605 | 4886 |
| TESTI2025924 | 4580 | 163 . . . 2028 | 4887 |
| TESTI2026647 | 4581 | 753 . . . 1556 | 4888 |
| TESTI2029252 | 4582 | 191 . . . 2293 | 4889 |
| TESTI2032643 | 4583 | 1810 . . . 2334 | 4890 |
| TESTI2034251 | 4584 | 188 . . . >1827 | 4891 |
| TESTI2035981 | 4585 | 814 . . . 1155 | 4892 |
| TESTI2036288 | 4586 | 86 . . . 475 | 4893 |
| TESTI2037830 | 4587 | 911 . . . 1228 | 4894 |
| TESTI2039060 | 4588 | 140 . . . 2080 | 4895 |
| TESTI2049956 | 4589 | 438 . . . 1856 | 4896 |
| TESTI2050780 | 4590 | 70 . . . 1470 | 4897 |
| TESTI4000137 | 4591 | 396 . . . 2435 | 4898 |
| TESTI4000155 | 4592 | 72 . . . 3518 | 4899 |
| TESTI4000183 | 4593 | 258 . . . 1160 | 4900 |
| TESTI4000214 | 4594 | 60 . . . 3710 | 4901 |
| TESTI4000319 | 4595 | 1609 . . . 2916 | 4902 |
| TESTI4001984 | 4596 | 651 . . . 1166 | 4903 |
| TESTI4005317 | 4597 | 607 . . . 2406 | 4904 |
| TESTI4006473 | 4598 | 166 . . . 4188 | 4905 |
| TESTI4008058 | 4599 | 849 . . . 2573 | 4906 |
| TESTI4008302 | 4600 | 933 . . . 2537 | 4907 |
| TESTI4010382 | 4601 | 160 . . . 4062 | 4908 |
| TESTI4011070 | 4602 | 978 . . . >3811 | 4909 |
| TESTI4011072 | 4603 | 255 . . . >3737 | 4910 |
| TESTI4011829 | 4604 | 2254 . . . >4540 | 4911 |
| TESTI4013365 | 4605 | 858 . . . 2717 | 4912 |
| TESTI4013602 | 4606 | 3496 . . . 3990 | 4913 |
| TESTI4013894 | 4607 | 367 . . . 1077 | 4914 |
| TESTI4014801 | 4608 | 1383 . . . 2555 | 4915 |
| TESTI4015012 | 4609 | 1699 . . . 3558 | 4916 |
| TESTI4015442 | 4610 | 12 . . . 3323 | 4917 |
| TESTI4017714 | 4611 | 318 . . . 2501 | 4918 |
| TESTI4019657 | 4612 | 1789 . . . 2925 | 4919 |
| TESTI4021482 | 4613 | 1 . . . 507 | 4920 |
| TESTI4024387 | 4614 | 404 . . . 1063 | 4921 |
| TESTI4025268 | 4615 | 3 . . . 1262 | 4922 |
| TESTI4025494 | 4616 | 282 . . . 2381 | 4923 |
| TESTI4025547 | 4617 | 5 . . . 964 | 4924 |
| TESTI4025865 | 4618 | 45 . . . 1421 | 4925 |
| TESTI4026207 | 4619 | 96 . . . 2540 | 4926 |
| TESTI4028938 | 4620 | 200 . . . 1711 | 4927 |
| TESTI4028958 | 4621 | 1336 . . . 2502 | 4928 |
| TESTI4029348 | 4622 | 116 . . . 613 | 4929 |
| TESTI4029528 | 4623 | 46 . . . 2544 | 4930 |
| TESTI4029690 | 4624 | 165 . . . 917 | 4931 |
| TESTI4031745 | 4625 | 1636 . . . 4002 | 4932 |
| TESTI4032090 | 4626 | 198 . . . 998 | 4933 |
| TESTI4032112 | 4627 | 815 . . . 1654 | 4934 |
| TESTI4036767 | 4628 | 62 . . . 1039 | 4935 |
| TESTI4038721 | 4629 | 2156 . . . 3292 | 4936 |
| TESTI4041086 | 4630 | 1946 . . . 3313 | 4937 |
| TESTI4046240 | 4631 | 981 . . . 1469 | 4938 |
| THYMU2004139 | 4632 | 407 . . . >2108 | 4939 |
| THYMU2004284 | 4633 | 922 . . . 1344 | 4940 |
| THYMU2006001 | 4634 | 230 . . . 1663 | 4941 |
| THYMU2028739 | 4635 | 408 . . . 1925 | 4942 |
| THYMU2030462 | 4636 | 1324 . . . 1725 | 4943 |
| THYMU2031139 | 4637 | 382 . . . 1890 | 4944 |
| THYMU2031249 | 4638 | 643 . . . 1713 | 4945 |
| THYMU2032976 | 4639 | 102 . . . 482 | 4946 |
| THYMU2033401 | 4640 | 221 . . . 646 | 4947 |
| THYMU2034279 | 4641 | 1718 . . . 2176 | 4948 |
| THYMU2035078 | 4642 | 396 . . . 902 | 4949 |
| THYMU2035710 | 4643 | 988 . . . 1443 | 4950 |
| THYMU2040925 | 4644 | 179 . . . 727 | 4951 |
| THYMU3000269 | 4645 | 1966 . . . 2742 | 4952 |
| THYMU3000360 | 4646 | 792 . . . 1241 | 4953 |
| THYMU3001428 | 4647 | 486 . . . 2294 | 4954 |
| TKIDN2008778 | 4648 | 1512 . . . 1862 | 4955 |
| TKIDN2012771 | 4649 | 2185 . . . 3315 | 4956 |
| TKIDN2018926 | 4650 | 59 . . . 388 | 4957 |
| TLIVE2001684 | 4651 | 1046 . . . 2137 | 4958 |
| TLIVE2002046 | 4652 | 255 . . . 1334 | 4959 |
| TLIVE2007607 | 4653 | 220 . . . 1746 | 4960 |
| TRACH1000212 | 4654 | 32 . . . 3826 | 4961 |
| TRACH2000862 | 4655 | 259 . . . 2160 | 4962 |
| TRACH2007483 | 4656 | 756 . . . 3095 | 4963 |
| TRACH2019672 | 4657 | 289 . . . 1350 | 4964 |
| TRACH2024408 | 4658 | 392 . . . >2211 | 4965 |
| TRACH2024559 | 4659 | 1450 . . . 1905 | 4966 |
| TRACH3000134 | 4660 | 293 . . . 2488 | 4967 |
| TRACH3000420 | 4661 | 17 . . . 3577 | 4968 |
| TRACH3002561 | 4662 | 2181 . . . 2603 | 4969 |
| TRACH3003683 | 4663 | 1157 . . . 1690 | 4970 |
| TRACH3003832 | 4664 | 6 . . . 2798 | 4971 |
| TRACH3007866 | 4665 | 183 . . . 2450 | 4972 |
| TUTER2000057 | 4666 | 27 . . . 833 | 4973 |
| UTERU2004299 | 4667 | 452 . . . 934 | 4974 |
| UTERU2008040 | 4668 | 286 . . . 1521 | 4975 |
| UTERU2011220 | 4669 | 453 . . . 842 | 4976 |
| UTERU2019534 | 4670 | 551 . . . 1021 | 4977 |
| UTERU2021820 | 4671 | 1545 . . . 2096 | 4978 |
| UTERU2028734 | 4672 | 217 . . . 1956 | 4979 |
| UTERU2032279 | 4673 | 1252 . . . 2037 | 4980 |
| UTERU2033577 | 4674 | 164 . . . 1009 | 4981 |
| UTERU2035978 | 4675 | 56 . . . 436 | 4982 |
| UTERU3000402 | 4676 | 798 . . . 1598 | 4983 |
| UTERU3000738 | 4677 | 792 . . . 1547 | 4984 |
| UTERU3001053 | 4678 | 2485 . . . >3535 | 4985 |
| UTERU3014791 | 4679 | 2452 . . . 3027 | 4986 |
| UTERU3015069 | 4680 | 2538 . . . 3986 | 4987 |
| UTERU3015412 | 4681 | 67 . . . 1464 | 4988 |
| UTERU3017176 | 4682 | 3512 . . . >3913 | 4989 |
| TESTI4038779 | 4683 | 202 . . . 1971 | 4990 |

Primers used to synthesize polynucleotides can be designed based on the nucleotide sequences of polynucleotides of the present invention, shown in SEQ ID NOs in Table 1 above. When synthesizing full-length cDNAs, an oligo dT primer can be used as the 3′-end primer. The length of the primer is usually 15-100 bp, and favorably between 15-35 bp. In the case of LA PCR, described below, a primer length of 25-35 bp provides a good result.

Methods for designing a primer that enables specific amplification based on a target nucleotide sequence are known to those skilled in the art (Current Protocols in Molecular Biology, Ausubel et al. edit, (1987) John Wiley & Sons, Section 6.1-6.4). In principle, primers based on 5′-end sequences are designed such that amplification products will include the translation start site. Accordingly, for example, when the 5′-end primer is designed based on the nucleotide sequence of the 5′ untranslated region (5′UTR), any part of the 5′-end, which ensures specificity to the cDNA of interest, can be selected as the primer.

When synthesizing a full-length cDNA, the target nucleotide sequence to be amplified can extend to several thousand bp in some cDNA. Such long nucleotides can be amplified using methods such as LA PCR (Long and Accurate PCR). The use of LA PCR is advantageous when synthesizing long DNA. In LA PCR, in which a special DNA polymerase having 3′->5′ exonuclease activity is used, misincorporated nucleotides can be removed. Accordingly, accurate synthesis of the complementary strand can be achieved even with a long nucleotide sequence. By using LA PCR, amplification of nucleotides 20 kb or longer can be achieved under desirable conditions (Takeshi Hayashi (1996) Jikken-Igaku Bessatsu, “Advanced Technologies in PCR” Youdo-sha).

Template DNAs for synthesizing the full-length cDNAs of the present invention can be obtained by using cDNA libraries prepared by various methods. The full-length cDNA clones of the present invention are clones with a high probability of completeness in length, obtained by a method comprising the steps of [1] preparing libraries containing cDNAs with a very high fullness ratio using oligo-capping, and [2] assembling 5′-end sequences and selecting those with the highest probability of completeness in length in clusters formed (there are many clones longer in the 5′-end direction).

However, the use of primers designed based on the full-length nucleotide sequences provided by the present invention enable full-length cDNAs to be easily obtained without using such a special technique.

The problem with commercially available cDNA libraries or those prepared by known methods is that mRNA contained in these libraries has a very low fullness ratio. Thus, it is difficult to screen full-length cDNA clones directly from the library using ordinary cloning methods. The present invention has revealed nucleotide sequences of novel full-length cDNA. If such a full-length nucleotide sequence is provided, it is possible to synthesize a target full-length cDNA by using enzymatic reactions such as PCR. In particular, a full-length-enriched cDNA library, synthesized by methods such as oligo-capping, is desirable to more reliably synthesize a full-length cDNA.

The present invention provides isolated polynucleotides comprising the nucleotide sequences of SEQ ID NO: 1 as shown in Table 1, or homologs thereof. As used herein, an “isolated polynucleotide” is a polynucleotide whose structure is not identical to that of any naturally occurring polynucleotide or to that of any fragment of a naturally occurring genomic polynucleotide spanning more than three separate genes. The term therefore includes, for example, (a) a DNA which comprises the sequence of part of a naturally occurring genomic DNA molecule in the genome of the organism in which it naturally occurs; (b) a polynucleotide incorporated into a vector or into the genomic DNA of a prokaryote or eukaryote such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion polypeptide. Specifically excluded from this definition are polynucleotides of DNA molecules present in mixtures of different (i) DNA molecules, (ii) transfected cells, or (iii) cell clones; e.g., as these occur in DNA libraries such as cDNA or genomic DNA libraries.

The 5′-end sequence of the full-length cDNA clones of this invention can be used to isolate the regulatory elements of transcription, including the promoter on the genome. A rough draft of the human genome (an analysis of the human genomic sequence with lower accuracy), which covers 90% of the genome, has been reported (Nature, Vol. 409, 814-823, 2001), and by the year 2003, analysis of the entire human genomic sequence will be finished. However, using software to analyze transcription start sites on the human genome, in which long introns exist, is difficult. In contrast, it is easy to specify transcription start sites in the genomic sequence using nucleotide sequences which include the 5′-end of the full-length cDNA clones of the present invention, and thus it is easy to obtain genomic regions involved in transcription regulation, which include promoters contained upstream of the transcription start site.

The polypeptides encoded by the full-length cDNAs of the invention can be prepared as recombinant polypeptides or as natural polypeptides. For example, a recombinant polypeptide can be prepared by inserting a polynucleotide encoding a polypeptide of the present invention into a vector, introducing the vector into an appropriate host cell, and purifying the polypeptide expressed within that transformed host cell, as described below. In contrast, a natural polypeptide can be prepared, for example, by utilizing an affinity column to which is attached an antibody against a polypeptide of the present invention (Current Protocols in Molecular Biology (1987) Ausubel et al. edit, John Wiley & Sons, Section 16.1-16.19). The antibody used for affinity purification may be either a polyclonal antibody, or a monoclonal antibody. Alternatively, in vitro translation (see, for example, “On the fidelity of mRNA translation in the nuclease-treated rabbit reticulocyte lysate system.” Dasso M. C., and Jackson R. J. (1989) Nucleic Acids Res. 17: 3129-3144) may be used for preparing a polypeptide of the invention.

The present invention provides substantially pure proteins encoded by the full-length cDNAs of the present invention. The term “substantially pure” herein used in reference to a given protein or polypeptide means that the protein or polypeptide is substantially free from other biological macromolecules. For example, the substantially pure protein or polypeptide is at least 75%, 80%, 85%, 95%, or 99% pure by dry weight. Purity can be measured by any appropriate standard method known in the art, for example, by column chromatography, polyacrylamide gel electrophoresis, or HPLC analysis.

Polypeptides functionally equivalent to the polypeptides of the present invention can be prepared based on the activities of the polypeptides of the present invention, clarified in the above-mentioned manner. Whether or not a particular polypeptide is functionally equivalent to a polypeptide of the present invention can be verified by using the biological activity of the polypeptide of the present invention as an index to examine whether that polypeptide has the said activity.

Polypeptides functionally equivalent to polypeptides of the present invention can be prepared by those skilled in the art, for example, by using a method for introducing mutations into a polypeptide amino acid sequence (for example, site-directed mutagenesis (Current Protocols in Molecular Biology, edit, Ausubel et al., (1987) John Wiley & Sons, Section 8.1-8.5). Such polypeptides can also be generated by spontaneous mutations. The present invention also includes polypeptides comprising the amino acid sequences shown in Table-1 in which one or more amino acids are substituted, deleted, inserted, and/or added, as long as the polypeptides have a function equivalent to that of a polypeptide identified in the Examples of the present invention, described later.

There are no limitations as to the number and site of amino acid mutation, as long as polypeptide function is maintained. The number of mutations typically corresponds to 30% or less, or 20% or less, or 10% or less, preferably 5% or less or 3% or less of the total amino acids, more preferably 2% or less or 1% or less of the total amino acids. Alternatively, herein, substitution of one or more amino acids includes substitution of several amino acids. As used herein, the term “several amino acids” means, for example, five amino acids, preferably four or three amino acids, more preferably two amino acids, and further preferably one amino acid.

Herein “conservative amino acid substitution” refers to substitution of an amino acid residue belonging to a group comprising a chemically similar side chain, with another amino acid in the same group. Groups of amino acid residues having similar side chains have been defined in the art. These groups include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine) nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In addition, polypeptides functionally equivalent to the polypeptides of the present invention can be isolated by using techniques of hybridization or gene amplification known to those skilled in the art. Specifically, using hybridization (Current Protocols in Molecular Biology, edit, Ausubel et al., (1987) John Wiley & Sons, Section 6.3-6.4)), those skilled in the art can usually isolate a polynucleotide highly homologous to a polynucleotide encoding the polypeptide identified in the present Example, based on the identified nucleotide sequence (Table 1) or a portion thereof, and obtain a functionally equivalent polypeptide from the isolated polynucleotide. The present invention includes polypeptides encoded by polynucleotides which hybridize with the polynucleotides encoding the polypeptides identified in the present Example, as long as the polypeptides are functionally equivalent to those identified in the present Example. Organisms from which the functionally equivalent polypeptides are isolated include but are not limited to vertebrates such as humans, mice, rats, rabbits, pigs and cows.

Washing conditions for hybridization for polynucleotide isolation encoding functionally equivalent polypeptides are usually “1×SSC, 0.1% SDS, 37° C.”; more stringent conditions are “0.5×SSC, 0.1% SDS, 42° C.”; and still more stringent conditions are “0.1×SSC, 0.1% SDS, 65° C.”. Alternatively, the following conditions can be given as hybridization conditions of the present invention. Namely, conditions in which hybridization is performed at “6×SSC, 40% Formamide, 25° C.”, and washing at “1×SSC, 55° C.” can be given. More preferable conditions are those in which hybridization is performed at “6×SSC, 40% Formamide, 37° C.”, and washing at “0.2×SSC, 55° C.”. Even more preferable are those in which hybridization is performed at “6×SSC, 50% Formamide, 37° C.”, and washing at “0.1×SSC, 62° C.”. The more stringent the hybridization conditions, the more frequently polynucleotides highly homologous to the probe sequence are isolated. Therefore, it is preferable to conduct hybridization under stringent conditions. Examples of stringent conditions in the present invention are, washing conditions of “0.5×SSC, 0.1% SDS, 42° C.”, or alternatively, hybridization conditions of “6×SSC, 40% Formamide, 37° C.”, and washing at “0.2×SSC, 55° C.”.

One skilled in the art can suitably select various conditions, such as SSC dilution ratio, formamide concentration, and temperature, to accomplish a similar stringency.

The above-mentioned combinations of SSC, SDS, and temperature conditions are indicated just as examples. Those skilled in the art can select hybridization conditions with similar stringency to those mentioned above by properly combining the above-mentioned or other factors that determine hybridization stringency (for example, probe concentration, probe length, and duration of hybridization reaction).

The amino acid sequences of polypeptides isolated by using hybridization techniques usually have high identity to those of the polypeptides of the present invention, shown in Table 1. The present invention encompasses polynucleotides comprising a nucleotide sequence with high identity to the nucleotide sequence of claim 1 (a). Furthermore, the present invention encompasses peptides, or polypeptides comprising an amino acid sequence with high identity to the amino acid sequence encoded by the polynucleotide of claim 1 (b). The term “high identity” indicates sequence identity of at least 40% or more; preferably 60% or more; and more preferably 70% or more. Even more preferable is identity of 90% or more, 93% or more, or 95% or more. Further more preferable is 97% or more, or 99% or more. Identity can be determined using the BLAST search algorithm.

As used herein, “percent identity” of amino acid sequences or nucleic acids is determined using the BLAST algorithm of Karlin and Altschul (Proc. Natl. Acad. Sci. USA 90:5873-5877, 1993). Such an algorithm is incorporated into the BLASTN and BLASTX programs of Altschul et al. (J. Mol. Biol. 215:403-410, 1990). BLAST nucleotide searches are performed with the BLASTN program, using for example, score=100, wordlength=12. BLAST protein searches are performed with the BLASTX program, using for example, score=50, wordlength=3. When utilizing the BLAST and Gapped BLAST programs, the default parameters of each program are used. See http://www.ncbi.nlm.nih.gov.

Using the gene amplification technique (PCR) (Current Protocols in Molecular Biology, edit, Ausubel et al., (1987) John Wiley & Sons, Section 6.1-6.4)) and primers designed based on the nucleotide sequences (Table 1) or portions thereof as identified in the present Example, it is possible to isolate polynucleotide fragments highly homologous to the polynucleotide sequences or portions thereof, and to obtain polypeptides functionally equivalent to a particular polypeptide identified in the present Example, based on the isolated polynucleotide fragment.

The present invention also provides polynucleotides containing at least 15 nucleotides complementary to a polynucleotide comprising a nucleotide sequence of SEQ ID NOs shown in Table 1, or the complementary strand thereof. Herein, the term “complementary strand” is defined as the other strand to one strand of a double stranded DNA composed of A:T and G:C base pairs. In addition, “complementary” is not only defined as sequences completely matching a continuous region of at least 15 nucleotides, but is also defined to include sequences comprising identity of at least 70%, favorably 80% or higher, more favorably 90% or higher, and most favorably 95% or higher within that region. Identity may be determined using the algorithm described herein.

Such polynucleotides includes probes and primers used for the detection and amplification of polynucleotides encoding the inventive polypeptides. When used as a primer, such a polynucleotide usually comprises 15 to 100 bp, and preferably of 15 to 35 bp. When used as a probe, such a polynucleotide comprises the whole or a part of the sequence of a polynucleotide of the present invention, and comprises at least 15 bp. When used as a primer, such a polynucleotide is complementary at the 3′-end, and restriction enzyme recognition sequences or tags can be added to the 5′-end.

Furthermore, polynucleotides of the present invention include antisense polynucleotides for suppressing the expression of a polypeptide of the present invention, which comprises an amino acid sequence of SEQ ID NOs shown in Table 1. To exert an antisense effect, an antisense polynucleotide has at least 15 bp or more, for example 50 bp or more, preferably 100 bp or more, and more preferably 500 bp or more, and usually has 3000 bp or less, and preferably 2000 bp or less. Antisense polynucleotides can be used in gene therapy of diseases caused by abnormalities of the polypeptides of the invention (abnormal function or abnormal expression). An antisense polynucleotide can be prepared, for example, using the phosphorothioate method (“Physicochemical properties of phosphorothioate oligodeoxynucleotides.” Stein (1988) Nucleic Acids Res. 16: 3209-3221) based on the sequence information of polynucleotides encoding a polypeptide of this invention (for example, the nucleotide sequences of SEQ ID NOs: 1 to 2188 and SEQ ID NOs: 4377 to 4683).

The present invention also includes polynucleotides that can use ribozyme or RNA interference (RNAi) activity to downregulate expression of a polynucleotide of the present invention, where such a polynucleotide can be designed based on the nucleotide sequence of the polynucleotide of the present invention.

A ribozyme is a polynucleotide that comprises 1) an antisense sequence of a polynucleotide of the present invention, and 2) the nucleotide sequence of a catalytic unit required for catalytic action. The antisense sequence constituting the ribozyme can be appropriately selected to be compatible with the structure of the ribozyme's catalytic unit. The ribozome's catalytic unit is well known in the art. For example, the hammer-head ribozyme (Rossi et al. (1991) Pharmac. Ther. 50: 245-254) and hairpin ribozyme (Hampel et al. (1990) Nucl. Acids Res. 18: 299-304, and U.S. Pat. No. 5,254,678) are known to have nucleotide sequence-specific cleaving activity. These ribozymes use this catalytic activity to cleave, at a specific position, polynucleotides which hybridize to the antisense sequence.

For example, the autolytic domain of a hammer-head ribozyme cleaves on the 3′ side of C15 in the sequence G13U14C15. Base pairing between U14 and A9 plays an important role in this activity, and A15 or U15 can be cleaved instead of C15 (Koizumi M, et al: FEBS Lett 228: 228, 1988). A restriction enzyme-like RNA-cleaving ribozyme that recognizes the target RNA sequences UC, UU, or UA can be produced by designing the ribozyme such that the substrate binding site complements the RNA sequence near the target site (Koizumi, M. et al., FEBS Lett, 239:285, 1988; Koizumi, M. and Otsuka, E., Protein, Nucleic acid, and Enzyme, 35:2191, 1990; Koizumi, M. et al., Nucl Acids Res, 17:7059, 1989). For example, the polynucleotides of the present invention (SEQ ID NOs: 1-2188 and SEQ ID NOs: 4377-4683) contain a number of potential target sites. The polynucleotides of the present invention can be cleaved at desired positions with a ribozyme which contains an appropriately selected antisense sequence.

The ribozyme preferably comprises RNA and may be synthesized chemically or produced by enzymatic reaction. Methods of chemical synthesizing RNA are known in the art. Alternatively, the ribozyme can be produced by using RNA polymerase to transcribe a polynucleotide encoding the ribozyme. To produce a ribozyme by transcription, a polynucleotide encoding the ribozyme is arranged downstream of a promotor recognized by an RNA polymerase. Such RNA polymerases include T7 RNA polymerase and SP6 RNA polymerase. Alternatively, a ribozyme can be expressed in a host cell by inserting a polynucleotide encoding the ribozyme into an appropriate vector, and then introducing the vector into a host cell. The vector contains a promotor that can direct the expression of the gene in the host cell.

The present invention also provides siRNA (small interfering RNA) that downregulates the expression of a polynucleotide of the present invention. siRNA is a technique for controlling gene expression that inhibits protein synthesis from an mRNA by using a double-stranded RNA which comprises the same nucleotide sequence as that mRNA (Fire et al. (1998) Nature 391: 806-811). The effect of downregulating gene expression using double-stranded RNA is called “the RNAi effect”. siRNA has been reported to effectively control gene expression in mice (Zamore et al. (2000) Cell 101:25-33; Gura (2000) Nature 404: 804-808). Thus the introduction of such a double-stranded RNA into cells can result in selective downregulation of target gene expression.

There is no limitation as to the length of the siRNAs. The double-stranded RNAs introduced into cells are enzymatically digested inside these cells, starting from their original 3′ end and forming fragments of 21 bp to 23 bp. The enzyme that digests the double-stranded RNA is called ‘dicer’. The resulting double-stranded RNA fragments recognize and bind to target nucleotide sequences which comprise the same sequence. The nucleotide sequence is then cleaved by the activity of RNase III-like nuclease (Hammond et al. (2000) Nature, 404: 293-298; Zamore et al. (2000) Cell 101: 25-33).

siRNA is introduced into cells to downregulate gene expression using RNAi activity. siRNA can be introduced into cells using the same methods as for ribozymes. Specifically, chemically-synthesized, double-stranded RNA can be introduced into cells. When synthesized RNA, including antisense RNA and siRNA, is intended for introduction into cells, it can be pre-modified to prevent degradation by nuclease. For example, thiolated RNA is protected from nuclease degradation.

Alternatively, siRNA can be expressed in cells. For example, siRNA can be expressed in cells by inserting a sense sequence and its corresponding antisense sequence into a vector, and then transforming cells with that vector. When the two strands are adjacent, the expressed double-stranded RNA will have a hairpin-loop structure. When the two strands are expressed under the control of different promotors, the resulting double-stranded RNA will comprise two separate strands. Promotors generally used for the expression of siRNA include the U6 promotor.

The nucleotide sequence of an antisense polynucleotide, ribozyme, or siRNA of the present invention may be completely identical or complementary to any one of the nucleotide sequences of SEQ ID NOs: 1-2188 and SEQ ID NOs: 4377-4683, or may have high homology to these nucleotide sequences. The phrase “high homology” to an antisense polynucleotide, ribozyme, or siRNA nucleotide sequence typically means 90% or higher homology, preferably 95% or higher homology, more preferably 98% or higher homology, and still more preferably 99% or higher homology. The homology of a nucleotide sequence can be estimated, for example, by a method described herein.

One skilled in the art can design siRNA based on the nucleotide sequence of a gene whose expression is to be downregulated. The typical methods for designing siRNA include, for example, those described below. To begin with, it is advantageous to avoid using as target sequences: 1) 5′- or 3′-untranslated regions, and 2) regions adjacent to the start codon.

These regions often serve as binding regions for transcriptional regulatory proteins. In addition, these regions may also contain nucleotide sequences conserved among various mRNAs, and thus they may act to inhibit the expression of genes other than the gene of interest.

Thus, it may be advantageous to arrange the target sequence, for example, within the ORF downstream of the start codon. It is preferable to adjust the number of nucleotides between the start codon and the target sequence, for example, to 50 nucleotides or more. Typically, the nucleotide sequence of an siRNA is designed so that it starts from an aa sequence and comprises 19-21 consecutive nucleotides. A dinucleotide overhang is added to one end of siRNA. The nucleotide sequence of such an overhang may include doublets, dTdT or UU sequences. The GC content of a nucleotide sequence constituting siRNA is preferably about 50%. G and C nucleotide residues are preferably uniformly distributed throughout the siRNA.

The action of siRNA is based on sequence-specific mRNA hybridization. Thus, to achieve downregulation specific to a particular gene, it is essential to make the target nucleotide sequence as specific as possible to that gene. It is thus preferable to use homology searches to confirm that the proposed target nucleotide sequence exhibits negligible homology with other genes. Nucleotide sequence homology can be determined using established algorithms.

As long as an siRNA of the present invention down-regulates the expression of a polynucleotide of the present invention, it is not limited to nucleotide sequences obtained using the typical design method described above. For example, even if the target sequence is not specific to the nucleotide sequence of a particular gene, it can specifically downregulate the expression of a gene of interest in cells which do not express genes comprising homologous nucleotide sequences. Furthermore, double-stranded RNA having RNAi activity can be obtained without using the above-described methods typically used to select a target sequence.

The polynucleotides or antisense polynucleotides, ribozymes, and siRNAs of the present invention can be used in, for example, gene therapy. Preferable target diseases may be, for example, cancers or various inflammatory diseases. Such molecules can be used for gene therapy, for example, by administrating them to patients in vivo or ex vivo using viral vectors such as retroviral vectors, adenoviral vectors, and adeno-related viral vectors, or non-viral vectors such as liposomes.

The present invention also includes partial peptides of the polypeptides of the invention. Such a partial peptide comprises a polypeptide generated as a result of removing a signal peptide from a secretory protein. If a polypeptide of the present invention has activity as a receptor or ligand, the partial peptide may function as a competitive inhibitor of the polypeptide, and may bind to the receptor (or ligand). In addition, the present invention includes an antigen peptide for raising antibodies. For the peptides to be specific to a polypeptide of the present invention, the peptides comprise at least seven amino acids, preferably eight amino acids or more, more preferably nine amino acids or more, and even more preferably ten amino acids or more. The peptide can be used to prepare an antibody against or competitive inhibitor of a polypeptide of the present invention, and can also be used to screen for a receptor that binds to the polypeptide of this invention. The partial peptides of this invention can be produced, for example, by genetic engineering methods, known methods for synthesizing peptides, or by digesting a polypeptide of the invention with an appropriate peptidase.

The present invention also relates to a vector into which a polynucleotide of the invention is inserted. Vectors of the present invention are not limited as long as they can contain the inserted polynucleotide stably. For example, if E. coli is used as a host, vectors such as pBluescript vector (Stratagene) are preferred cloning vectors. To produce a polypeptide of the invention, expression vectors are especially useful. Any expression vector can be used as long as it is capable of expressing the polypeptide in vitro, in E. coli, in cultured cells, or in vivo. For example, pBEST vector (Promega) is preferable for in vitro expression, pET vector (Invitrogen) for E. coli, pME18S-FL3 vector (GenBank Accession No. AB009864) for cultured cells, and pME18S vector (Mol. Cell. Biol. (1988) 8: 466-472) for in vivo expression. To insert a polynucleotide of the present invention, ligation utilizing restriction sites can be performed according to standard methods (Current Protocols in Molecular Biology (1987) Ausubel et al. edit, John Wiley & Sons, Section 11.4-11.11).

The GATEWAY™ system (Invitrogen), which is an expression vector construction system for polypeptide expression, can also be used (Experimental Medicine, Vol. 18, No. 19 (December), p2716-2717, 2000). This system includes two types of site-specific recombinases (BP CLONASE™ and LR CLONASE™) derived from lambda phage and uses BP CLONASE™-specific recombination sites for the Entry Vector, and LR CLONASE™-specific recombination sites for the Destination Vector, which may comprise a tag useful for polypeptide purification. With this system, an expression vector can be obtained by using homologous recombination.

First, a polynucleotide fragment of interest is inserted into the entry vector using the first recombination. Then, a second recombination is allowed to take place between the entry vector, where the polynucleotide fragment of interest has been inserted, and the destination vector. Thus, the expression vector can be prepared rapidly and efficiently. Using the above-mentioned typical restriction enzyme/ligase reaction method, expression vector construction and expression of a polypeptide of interest takes about seven to ten days. However, using the GATEWAY™ system, the polypeptide of interest can be expressed and prepared in only three to four days. Thus, the system ensures a high-throughput functional analysis for expressed polypeptides (http://biotech.nikkeibp.co.jp/netlink/lto/gateway/).

The present invention also relates to a transformant carrying a vector of the present invention. Any cell can be used as a host into which a vector of this invention is inserted, and various kinds of host cells can be used depending on the purpose. For example, COS cells or CHO cells can be used for strong expression of the polypeptide in eukaryotic cells.

Introduction of such a vector into host cells can be performed, for example, by calcium phosphate precipitation, electroporation (Current Protocols in Molecular Biology (1987) Ausubel et al. edit, John Wiley & Sons, Section 9.1-9.9), lipofectamine method (GIBCO-BRL), or microinjection, etc.

Further, a polynucleotide containing at least 15 nucleotides comprising a nucleotide sequence of any one of the polynucleotides comprising the nucleotide sequences of SEQ ID NOs shown in Table 1, or the complementary strand thereof, can be used not only as a primer for synthesizing full-length cDNAs, but also for testing and diagnosing abnormalities of the polypeptide encoded by the full-length cDNA of the present invention. For example, by utilizing polymerase chain reaction (genomic DNA-PCR, or RT-PCR) using a polynucleotide of this invention as a primer, a polynucleotide encoding a polypeptide of the invention can be amplified. It is also possible to obtain the regulatory region of expression in the 5′-upstream by using PCR or hybridization, since the transcription start site within the genomic sequence can be easily specified based on the 5′-end sequence of the full-length cDNA. The obtained genomic region can be used for detection and/or diagnosis of sequence abnormality using RFLP analysis, SSCP, or sequencing. Where expression of an mRNA of the present invention varies according to a specific disease, analysis of the amount of mRNA expression using a polynucleotide of the present invention as a probe or a primer enables detection and diagnosis of that disease.

The present invention also relates to antibodies that bind to a polypeptide of the present invention. There are no limitations as to the form of the antibodies of this invention. They include polyclonal antibodies, monoclonal antibodies, or portions thereof that can bind to an antigen. They also include antibodies of all classes. Furthermore, special antibodies such as humanized antibodies and chimeric antibodies are also included.

A polyclonal antibody of this invention can be obtained according to the standard method of synthesizing an oligopeptide corresponding to an amino acid sequence, and immunizing rabbits with that peptide (Current Protocols in Molecular Biology (1987) Ausubel et al. edit, John Wiley & Sons, Section 11.12-11.13). A monoclonal antibody of the present invention can be obtained according to the standard method of purifying a polypeptide expressed in E. coli, immunizing mice with that polypeptide, and producing a hybridoma cell by fusing spleen cells and myeloma cells (Current Protocols in Molecular Biology (1987) Ausubel et al. edit, John Wiley & Sons, Section 11.4-11.11).

An antibody binding to a polypeptide of the present invention can be used for purification of the polypeptide of the invention, and also for detection and/or diagnosis of abnormalities of the expression and structure of that polypeptide. Specifically, polypeptides can be extracted, for example, from tissues, blood, or cells, and a polypeptide of this invention can then be detected for the above purpose using Western blotting, immunoprecipitation, or ELISA.

Furthermore, an antibody binding to a polypeptide of the present invention can be utilized for treating a disease associated with that polypeptide. If the antibody is used to treat patients, human antibodies, humanized antibodies, or chimeric antibodies are preferred due to their low antigenicity. Human antibodies can be prepared by immunizing a mouse whose immune system is replaced with that of a human (e.g., see “Functional transplant of megabase human immunoglobulin loci recapitulates human antibody response in mice” Mendez, M. J. et al. (1997) Nat. Genet. 15: 146-156). These humanized antibodies can be prepared by recombination of the hypervariable region of a monoclonal antibody (Methods in Enzymology (1991) 203: 99-121).

The cDNAs (clones) of the present invention include polypeptide sequences encoding proteins whose function can be predicted, such as, for example, secretory and/or membrane proteins, glycoprotein-related proteins, signal transduction-related proteins, transcription-related proteins, disease-related proteins, enzyme and/or metabolism-related proteins, cell division- and/or cell proliferation-related proteins, cytoskeleton-related proteins, nuclear protein and/or RNA synthesis-related proteins, protein synthesis and/or transport-related proteins, cellular defense-related proteins, development and/or differentiation-related proteins, DNA- and/or RNA-binding proteins, ATP- and/or GTP-binding proteins. The results of cDNA homology searches can be used to estimate whether a cDNA sequence comprises the function of an above-described protein. Specifically, the function of a polypeptide encoded by a cDNA of the present invention can be inferred by (a) searching for a known gene or protein which is homologous to the complete or partial nucleotide sequence of the full-length cDNA of the present invention, and (b) comparing the function of the gene and that of the protein encoded by the gene.

Alternatively, the function of a polypeptide encoded by a cDNA of the present invention can be predicted when a signal sequence, transmembrane domain, nuclear translocation signal, glycosylation signal, phosphorylation site, zinc-finger motif, SH3 domain, or such is found in the amino acid sequence. In particular, partial sequence structures such as motif and domain structures are commonly found in a number of proteins, and comprise a minimal functional protein structure. The Pfam database identifies a total of 4,832 types of motifs and domains, including both those whose functions have been clarified and those whose functions remain unclear (http://www.sanger.ac.uk/Software/Pfam/index.shtml) Version 7.7 (the latest version as of December 2002).

A specific example of motif/domain function is shown below. The ITAM motif (immunoreceptor tyrosine-based activation motif) is found in the intracellular region of the T cell receptor which is expressed on the cell membrane of T cells participating in an immune response (Flaswinkel, H. et al. Semin Immunol 1995 February, 7(1):21-7). The ITAM motif has a tandem YXXL structure (tyrosine-arbitrary amino acid-arbitrary amino acid-leucine). On extracellular stimulus by an antigen or antibody, the tyrosine in the motif is phosphorylated by an enzyme (LCK) with a kinase domain. Then, ZAP70 binds to the phosphorylated tyrosine via the SH2 domain, resulting in downstream signal transduction (Bu, J. Y. et al., Proc Natl Acad Sci USA 1995 May 23, 92(11):5106-10; Neumeister, E. N. et al., Mol Cell Biol 1995 June, 15(6):3171-8).

A similar phenomenon has been found in mast cells as well as in T cells (Chen, T. et al., J Biol Chem 1996 Oct. 11, 271(41):25308-15). Thus, at the molecular level, such a phenomenon is the first step in the activation of immune cells in immunologic diseases such as allergies, atopic dermatitis, and asthma.

Even in a simple exemplary scheme such as the one described above, there are three major motif/domain structures—ITAM, the SH2 domain, and the protein kinase domain—each of which play an important role. The mechanism of this scheme can be interpreted using these three structures. Thus, collecting, categorizing and elucidating the function of molecules that comprise common motif/domain structures is exceedingly important in understanding the molecular-based mechanisms of various cellular functions, including and in addition to the immune response described herein. Searching for motif/domain structures is highly important as the first step in elucidating the functions of unknown polypeptides. It is also understood that an entire polypeptide structure is comprised by minimal structures such as motifs and domains, thus providing the overall function of an entire polypeptide.

The overall function of a polypeptide in cells can be accurately predicted at the molecular level using data obtained by domain and motif structure analysis. In addition, a fusion polypeptide comprising a partial amino acid sequence and a GFP protein or the like may be prepared, and then introduced into cultured cells. For example, if a polypeptide is localized on the cell membrane, it may function as a receptor or ion channel. Alternatively, if a polypeptide is localized in the nucleus, it can be predicted to serve as a polynucleotide-binding protein or to participate in transcription. Thus, the function of a polypeptide can also be predicted by determining its localization.

The function of a full-length cDNA obtained in the present invention can be predicted by carrying out the above-described analysis using its entire nucleotide sequence and the amino acid sequence it encodes. Even when the full-length cDNA nucleotide sequence is not available, a partial sequence thereof (preferably 300 nucleotides or more) often enables function to be predicted. However, function predicted based on information yielded in a partial sequence homology search will not necessarily be the same as that based on a full-length sequence. Functional prediction based on a full-length nucleotide sequence is obviously preferable.

A more specific method for predicting function involves homology searches of databases such as GenBank, Swiss-Prot, UniGene, nr and RefSeq, using BLAST or FASTA. The functions of polypeptides encoded by the cDNAs of the present invention can be predicted based on hit genes and the function of polypeptides encoded by these genes. Polypeptide functions can be predicted from the amino acid sequences deduced from the structure of the full-length nucleotide sequences. In this way, signal sequences and transmembrane domains can be predicted from amino acid sequences using PSORT [K. Nakai & M. Kanehisa, Genomics, 14: 897-911 (1992)], SOSUI [T. Hirokawa et al., Bioinformatics, 14, 378-379 (1998)] (Mitsui Knowledge Industry Co., Ltd.), MEMSAT [D. T. Jones, W. R. Taylor & J. M. Thornton, Biochemistry, 33, 3038-3049 (1994)], and the like. Alternatively, motifs and domains can be predicted from amino acid sequences by carrying out searches using Pfam, PROSITE (http://www.expasy.ch/prosite/), or such. The above-described procedures facilitate more accurate prediction of polypeptide function.

The databases GenBank, Swiss-Prot, UniGene, nr and RefSeq were searched as described above for homology to the 2,495 full-length clone sequences of the present invention whose full-length nucleotide had been determined (see Example 4 and the results of homology searches). In addition, the amino acid sequences deduced from the full-length nucleotide sequences were analyzed by database searches for signal sequences and transmembrane domains using PSORT and SOSUI (see Example 5). The clones were categorized into the fourteen functional categories shown below, based on 1) the results of annotation-based functional prediction (by referring to keywords in the hit data of Swiss-Prot, or to Definitions and Reference information in the hit data of GenBank, UniGene, nr or RefSeq), 2) the results of PSORT searches for signal sequences using the deduced ORFs and 3) the results of SOSUI searches for transmembrane domains using the deduced ORFs. As a result, 1,229 clones were estimated to encode proteins belonging to the categories described below.

Secretory and/or membrane protein (741 clones)

Glycoprotein-related protein (130 clones)

Signal transduction-related protein (111 clones)

Transcription-related protein (102 clones)

Disease-related protein (426 clones)

Enzyme and/or metabolism-related protein (230 clones)

Cell division- and/or cell proliferation-related protein (52 clones)

Cytoskeleton-related protein (61 clones)

Nuclear protein and/or RNA synthesis-related protein (58 clones)

Protein synthesis- and/or transport-related protein (72 clones)

Cellular defense-related protein (seven clones)

Development and/or differentiation-related protein (14 clones)

DNA- and/or RNA-binding protein (129 clones)

ATP- and/or GTP-binding protein (92 clones)

The clones predicted to belong to the category of secretory protein and/or membrane protein are the following 659 clones.

ACTVT2000380, ADIPS2000088, ADRGL2000172, ADRGL2003329, ADRGL2009146, ASTRO2014923, ASTRO3000301, BLADE1000176, BLADE2002073, BLADE2002947, BLADE2004462, BLADE2004670, BLADE2005036, BLADE2008539, BNGH42003570, BRACE1000186, BRACE2005457, BRACE2014306, BRACE2016981, BRACE2029112, BRACE2030884, BRACE2031527, BRACE2031531, BRACE2031899, BRACE2032385, BRACE2036005, BRACE2039249, BRACE2039327, BRACE2040138, BRACE2041200, BRACE2043142, BRACE2043665, BRACE2046295, BRACE3000697, BRACE3001391, BRACE3002298, BRACE3003004, BRACE3003595, BRACE3004058, BRACE3004113, BRACE3004772, BRACE3004843, BRACE3006462, BRACE3008137, BRACE3008384, BRACE3009574, BRACE3009708, BRACE3010397, BRACE3011271, BRACE3011505, BRACE3013740, BRACE3014005, BRACE3014068, BRACE3014807, BRACE3016884, BRACE3018963, BRACE3019084, BRACE3020286, BRACE3020594, BRACE3024662, BRACE3025531, BRACE3025630, BRACE3026008, BRACE3026735, BRACE3027326, BRACE3031838, BRACE3040856, BRALZ2016085, BRAMY2001473, BRAMY2004771, BRAMY2005052, BRAMY2017528, BRAMY2019300, BRAMY2019963, BRAMY2021498, BRAMY2028856, BRAMY2033003, BRAMY2033116, BRAMY2033594, BRAMY2036396, BRAMY2039872, BRAMY2040592, BRAMY2041542, BRAMY2045036, BRAMY2047420, BRAMY2047751, BRAMY2047765, BRAMY3002312, BRAMY3004224, BRAMY3004919, BRAMY3007206, BRAMY3007609, BRAMY3008505, BRAMY4000095, BRASW1000125, BRAWH1000127, BRAWH2002560, BRAWH2002761, BRAWH2007658, BRAWH2014414, BRAWH2014954, BRAWH2016221, BRAWH2016439, BRAWH2016702, BRAWH3000078, BRAWH3000314, BRAWH3001475, BRAWH3001891, BRAWH3002600, BRAWH3003555, BRAWH3003727, BRAWH3003992, BRAWH3004453, BRAWH3004666, BRAWH3005132, BRAWH3005912, BRAWH3006548, BRAWH3007221, BRAWH3007506, BRAWH3007592, BRAWH3008634, BRCAN2002948, BRCAN2006063, BRCAN2009203, BRCAN2010376, BRCAN2012355, BRCAN2012481, BRCAN2013655, BRCAN2014143, BRCAN2016619, BRCAN2024451, BRCOC2007034, BRCOC2019934, BRHIP2000691, BRHIP2001805, BRHIP2002172, BRHIP2004814, BRHIP2004883, BRHIP2005236, BRHIP2005752, BRHIP2009414, BRHIP2013699, BRHIP2026288, BRHIP3000526, BRHIP3007483, BRHIP3007586, BRHIP3008598, BRHIP3009448, BRHIP3015751, BRHIP3024118, BRHIP3026097, BRSSN2003086, BRSSN2004496, BRSSN2008549, BRSSN2011738, BRSSN2014424, BRSSN2018925, BRSTN2000872, BRSTN2003835, BRSTN2007000, BRSTN2010363, BRSTN2012380, BRSTN2015015, BRSTN2016470, BRSTN2016678, BRSTN2017110, BRTHA2002376, BRTHA2002493, BRTHA2002608, BRTHA2002808, BRTHA2003110, BRTHA2003461, BRTHA2005579, BRTHA2006075, BRTHA2008527, BRTHA2011194, BRTHA2012980, BRTHA2013460, BRTHA2015696, BRTHA2015878, BRTHA2016215, BRTHA2016496, BRTHA2017985, BRTHA2018344, BRTHA2018624, BRTHA3000633, BRTHA3002427, BRTHA3003474, BRTHA3007148, BRTHA3008386, BRTHA3008778, BRTHA3009037, BRTHA3009090, BRTHA3009291, BRTHA3016845, BRTHA3017047, BRTHA3017589, BRTHA3017848, BRTHA3018656, CERVX2002006, COLON2000568, COLON2002443, COLON2004478, COLON2005126, COLON2005772, CTONG1000302, CTONG1000341, CTONG1000488, CTONG1000508, CTONG2000042, CTONG2004062, CTONG2008233, CTONG2009423, CTONG2009531, CTONG2010803, CTONG2013178, CTONG2019652, CTONG2019788, CTONG2020127, CTONG2020522, CTONG2020638, CTONG2022601, CTONG2023512, CTONG2024749, CTONG2025496, CTONG2026920, CTONG2027327, CTONG2028124, CTONG2028687, CTONG3000707, CTONG3001370, CTONG3001560, CTONG3002020, CTONG3003179, CTONG3003483, CTONG3003737, CTONG3005648, CTONG3008252, CTONG3008258, CTONG3008496, CTONG3008566, CTONG3008951, CTONG3009227, CTONG3009239, CTONG3009328, CTONG3009385, D3OST2002182, D3OST2002648, DFNES1000107, DFNES2000146, DFNES2005266, DFNES2010502, FCBBF2001183, FCBBF2007510, FCBBF3003435, FCBBF3004502, FCBBF3009888, FCBBF3012170, FCBBF3021576, FCBBF3023895, FCBBF4000076, FEBRA1000030, FEBRA2007708, FEBRA2008311, FEBRA2008468, FEBRA2020668, FEBRA2025427, FEBRA2027082, HCASM2002502, HCASM2003212, HCASM2007047, HCHON2000212, HCHON2001084, HCHON2001548, HCHON2001577, HCHON2001712, HCHON2002676, HCHON2004007, HCHON2004776, HCHON2005921, HEART1000010, HEART2001680, HEART2010492, HLUNG2000014, HLUNG2003872, HLUNG2010464, HLUNG2015617, HLUNG2017350, HSYRA2005496, HSYRA2006873, HSYRA2008714, HSYRA2009102, IMR322002110, IMR322006222, KIDNE1000064, KIDNE2000832, KIDNE2000846, KIDNE2006580, KIDNE2010264, KIDNE2011635, KIDNE2012945, KIDNE2013095, LIVER2007415, LYMPB2000083, MESAN2001979, MESAN2012054, MESTC1000042, NHNPC2000606, NHNPC2001223, NOVAR2000136, NOVAR2001108, NT2RI2008724, NT2RI2009855, NT2RI2025909, NT2RI3001263, NT2RI3003095, NT2RI3003382, NT2RI3003409, NT2RI3005403, NT2RI3006171, NT2RI3006673, NT2RI3007065, NT2RI3007543, NT2RI3007978, NT2RP7000359, NT2RP7000466, NT2RP7004027, NT2RP7009030, NT2RP7014005, NTONG2000413, OCBBF2006151, OCBBF2006567, OCBBF2006764, OCBBF2007114, OCBBF2007428, OCBBF2009926, OCBBF2010140, OCBBF2017516, OCBBF2021788, OCBBF2024719, OCBBF2025458, OCBBF2030517, OCBBF2030574, OCBBF2031167, OCBBF2032590, OCBBF2033869, OCBBF2037598, OCBBF2038317, OCBBF3000483, OCBBF3003320, OCBBF3004314, PEBLM2000170, PEBLM2000338, PEBLM2002594, PEBLM2006113, PEBLM2007834, PERIC2001227, PERIC2003452, PERIC2003720, PERIC2004909, PERIC2005347, PERIC2006035, PERIC2007914, PLACE5000171, PLACE5000260, PLACE5000282, PLACE6012574, PLACE6019932, PLACE6020031, PLACE7000514, PLACE7001022, PROST1000184, PROST1000528, PROST1000559, PROST2003428, PROST2018090, PROST2018902, PROST2018922, PUAEN2002489, PUAEN2005588, PUAEN2006701, PUAEN2009174, PUAEN2009795, RECTM2000433, RECTM2001347, SKMUS2000757, SKNMC2002402, SMINT2002743, SMINT2009902, SMINT2011888, SMINT2015787, SPLEN2001599, SPLEN2009548, SPLEN2012889, SPLEN2015158, SPLEN2015267, SPLEN2015679, SPLEN2021701, SPLEN2023733, SPLEN2023791, SPLEN2025491, SPLEN2029522, SPLEN2029683, SPLEN2030335, SPLEN2030479, SPLEN2031125, SPLEN2031424, SPLEN2031547, SPLEN2031724, SPLEN2031780, SPLEN2032813, SPLEN2033098, SPLEN2034021, SPLEN2034781, SPLEN2036326, SPLEN2036821, SPLEN2037722, SPLEN2038180, SPLEN2038345, SPLEN2038407, SPLEN2040222, SPLEN2041304, SPLEN2042598, STOMA2004294, STOMA2008546, SYNOV2005817, SYNOV2012326, SYNOV2014400, SYNOV2016124, SYNOV4002883, SYNOV4003322, SYNOV4004184, SYNOV4004741, SYNOV4004914, SYNOV4006256, SYNOV4007430, SYNOV4007553, SYNOV4007671, SYNOV4008336, SYNOV4008440, TBAES2001258, TCERX2000613, TESOP2001345, TESOP2001865, TESOP2002273, TESOP2002539, TESOP2004114, TESOP2005485, TESOP2005579, TESOP2006041, TESOP2007052, TESOP2007262, TESOP2007636, TESTI1000257, TESTI1000348, TESTI2000644, TESTI2002036, TESTI2002618, TESTI2002928, TESTI2003347, TESTI2005610, TESTI2006648, TESTI2013382, TESTI2024567, TESTI2027019, TESTI2034767, TESTI2034953, TESTI2034997, TESTI2035997, TESTI2036684, TESTI2042450, TESTI2047071, TESTI2048898, TESTI2051767, TESTI2052822, TESTI4000215, TESTI4000724, TESTI4001100, TESTI4001527, TESTI4001561, TESTI4001665, TESTI4001923, TESTI4002552, TESTI4002754, TESTI4005805, TESTI4005961, TESTI4006053, TESTI4006137, TESTI4007064, TESTI4007163, TESTI4007239, TESTI4007382, TESTI4008050, TESTI4008401, TESTI4008429, TESTI4008797, TESTI4009608, TESTI4012448, TESTI4013369, TESTI4013667, TESTI4013830, TESTI4014392, TESTI4016238, TESTI4017575, TESTI4017901, TESTI4018835, TESTI4019566, TESTI4020092, TESTI4020102, TESTI4021478, TESTI4023722, TESTI4024420, TESTI4024874, TESTI4024890, TESTI4025797, TESTI4026456, TESTI4026785, TESTI4027821, TESTI4028062, TESTI4028429, TESTI4028823, TESTI4028880, TESTI4029836, TESTI4030159, TESTI4030505, TESTI4034172, TESTI4035065, TESTI4035649, TESTI4037244, TESTI4041053, TESTI4042711, TESTI4046487, TESTI4046819, THYMU2001053, THYMU2003632, THYMU2003760, THYMU2005003, THYMU2005303, THYMU2005321, THYMU2007658, THYMU2008725, THYMU2009425, THYMU2011548, THYMU2013386, THYMU2014353, THYMU2019210, THYMU2023711, THYMU2027497, THYMU2027695, THYMU2029676, THYMU2030068, THYMU2032035, THYMU2032437, THYMU2032655, THYMU2033079, THYMU2033308, THYMU2033816, THYMU2034314, THYMU2035064, THYMU2036085, THYMU2036459, THYMU2037226, THYMU2037348, THYMU2038772, THYMU2038797, THYMU2039780, THYMU2040412, THYMU2041015, THYMU3000028, THYMU3000036, THYMU3004835, THYMU3005696, THYMU3006168, THYMU3006811, THYMU3007137, THYMU3007368, THYMU3007845, TKIDN2002424, TKIDN2002632, TKIDN2006525, TKIDN2009092, TKIDN2009889, TKIDN2014771, TKIDN2019116, TLIVE2000023, TLIVE2001828, TLIVE2001927, TLIVE2002336, TLIVE2002690, TLIVE2003381, TLIVE2004110, TLIVE2008229, TOVAR2001281, TRACH1000205, TRACH2001549, TRACH2001684, TRACH2006387, TRACH2007059, TRACH2007834, TRACH2008300, TRACH2020525, TRACH2021964, TRACH2022553, TRACH2025535, TRACH2025911, TRACH3000014, TRACH3002064, TRACH3002168, TRACH3002650, TRACH3004786, TRACH3005294, TRACH3005549, TRACH3006149, TRACH3007391, TRACH3008629, TRACH3035199, TRACH3035526, TRACH3036193, TSTOM2000442, TSTOM2000553, TUTER2000916, UTERU1000339, UTERU2004688, UTERU2004929, UTERU2006137, UTERU2006568, UTERU2007444, UTERU2017762, UTERU2020718, UTERU2022020, UTERU2025025, UTERU2025645, UTERU2025891, UTERU2026090, UTERU2026203, UTERU2027591, UTERU2029953, UTERU2031851, UTERU2035323, UTERU2035469, UTERU3000645, UTERU3000899, UTERU3001240, UTERU3001571, UTERU3001585, UTERU3001652, UTERU3001988, UTERU3002209, UTERU3002383, UTERU3002786, UTERU3003116, UTERU3003776, UTERU3006308, UTERU3008671, UTERU3009690, UTERU3009979, UTERU3011063, UTERU3015500, UTERU3016789

The following 82 clones are also predicted to belong to the category of secretory protein and/or membrane protein.

BLADE2006830, BRACE2002589, BRACE2009318, BRACE2011677, BRACE2029396, BRACE2039823, BRACE2039832, BRAMY2019111, BRAMY2038516, BRAMY2045471, BRAWH2006395, BRAWH2008993, BRCOC2019841, BRHIP2003272, BRHIP2005271, BRHIP2005724, BRHIP2008389, BRHIP2026877, BRHIP3000240, BRTHA2011321, BRTHA2018011, BRTHA2018443, BRTHA3008826, CTONG2015633, CTONG2016942, CTONG2019822, FEBRA2000790, FEBRA2006519, FEBRA2028256, FEBRA2028516, HCASM2002754, HEART2009680, HLUNG2013350, HLUNG2015418, IMR322013396, LIVER2000247, NT2RI2009583, NT2RI2027157, NT2RP7008435, OCBBF2003327, OCBBF2030116, PLACE7000502, PROST2000452, PROST2019487, SPLEN2016932, SPLEN2037319, SYNOV2001660, SYNOV2013637, SYNOV4003981, SYNOV4005889, TBAES2000932, TESTI2015626, TESTI2029252, TESTI2032643, TESTI2039060, TESTI2050780, TESTI4000137, TESTI4000155, TESTI4006473, TESTI4011070, TESTI4013365, TESTI4013894, TESTI4014801, TESTI4032090, TESTI4041086, THYMU2004284, THYMU2030462, THYMU2033401, THYMU2034279, THYMU2035710, THYMU2040925, TKIDN2008778, TKIDN2012771, TKIDN2018926, TLIVE2007607, TRACH2019672, TRACH3000420, TRACH3003683, UTERU2011220, UTERU2021820, UTERU2032279, UTERU3015069

The clones predicted to belong to the category of glycoprotein-related protein are the following 115 clones.

ADIPS2000088, BNGH42003570, BRACE2005457, BRACE2014306, BRACE2029112, BRACE2039249, BRACE2046295, BRACE3001391, BRACE3011271, BRACE3016884, BRAMY2005052, BRAMY3004919, BRAMY4000095, BRAMY4000277, BRAWH1000127, BRAWH2007658, BRAWH2014414, BRAWH2016221, BRAWH3002600, BRCAN2006063, BRSSN2004496, BRTHA2008527, BRTHA2012980, BRTHA2016496, BRTHA3002427, BRTHA3017848, COLON2000568, COLON2004478, COLON2005772, CTONG1000341, CTONG2000042, CTONG2009423, CTONG2023512, CTONG2024749, CTONG2025496, CTONG3001370, CTONG3003737, D3OST2002648, DFNES2000146, DFNES2005266, FCBBF3012170, FEBRA1000030, FEBRA2008311, FEBRA2008468, HCHON2001712, HEART1000010, HEART2001680, HSYRA2005496, KIDNE2012945, LYMPB2000083, NESOP2001433, NOVAR2000136, NOVAR2001108, NT2RI3006171, NT2RI3006673, NT2RP7004027, OCBBF2033869, PLACE5000171, PROST1000184, PUAEN2009795, SMINT2010076, SMINT2011888, SMINT2015787, SPLEN2015267, SPLEN2021701, SPLEN2030335, SYNOV2005817, SYNOV2014400, SYNOV3000231, SYNOV3000302, TESOP2004114, TESOP2005485, TESTI1000257, TESTI2002036, TESTI2002618, TESTI2024567, TESTI2027019, TESTI4001527, TESTI4007163, TESTI4012406, TESTI4013830, TESTI4020092, TESTI4023546, TESTI4028823, TESTI4028880, TESTI4046819, THYMU2005303, THYMU2008725, THYMU2009425, THYMU2011548, THYMU2019210, THYMU2023711, THYMU2027497, THYMU2027695, THYMU2038797, THYMU3004835, TLIVE2003381, TRACH2006387, TRACH2007059, TRACH2022425, TRACH2022553, TRACH2022649, TRACH3002168, TRACH3008629, TRACH3035526, TSTOM2000442, UTERU2008347, UTERU2025025, UTERU2035469, UTERU3000899, UTERU3001240, UTERU3003116, UTERU3006308, UTERU3008671, UTERU3015500.

The following 15 clones are also predicted to belong to the category of glycoprotein-related protein.

BRAMY2019111, BRHIP2026877, BRTHA2018011, FEBRA2028256, HEART2009680, HLUNG2015418, NT2RI2009583, NT2RP7008435, OCBBF2003327, TESTI2032643, TESTI2039060, TESTI4011070, THYMU2035710, TRACH3003683, UTERU2032279

The clones predicted to belong to the category of signal transduction-related protein are the following 80 clones.

BNGH42007788, BRACE2008594, BRACE2030341, BRACE2044286, BRACE3002508, BRACE3003595, BRACE3006872, BRACE3011421, BRACE3015027, BRACE3027326, BRAMY2036567, BRAMY2038904, BRAMY3000213, BRAMY3002803, BRAMY3005091, BRAMY3005932, BRAMY4000095, BRAMY4000229, BRCAN2003703, BRCAN2014602, BRCAN2016619, BRCAN2028355, BRHIP2000819, BRHIP3025161, BRSSN2004719, BRSTN2008418, BRTHA2002281, BRTHA2015406, CTONG2006798, CTONG3000084, CTONG3002412, D3OST3000169, FCBBF3007540, HCASM2001301, HCHON2006250, HCHON2008112, HLUNG2002465, KIDNE2001847, NESOP2001694, NT2NE2003252, NT2RI2005166, NT2RI3007757, NT2RI3008652, NT2RP7005529, NT2RP7009147, NT2RP7013795, NT2RP8000483, OCBBF2004826, OCBBF2007028, OCBBF2022351, OCBBF2030354, OCBBF2037547, PLACE6019385, PLACE7008431, PROST2016462, PROST2018511, PUAEN2009852, SPLEN2036932, SYNOV2021320, TESOP2000801, TESOP2001166, TESTI2005739, TESTI2026505, TESTI2050137, TESTI4011745, TESTI4012505, TESTI4018208, TESTI4028059, THYMU2007060, THYMU2031046, THYMU2032014, THYMU2039305, THYMU3008436, TLIVE2001327, TRACH2009310, TRACH2025535, TRACH3009455, UTERU2025025, UTERU2036089, UTERU3016789

The following 31 clones are also predicted to belong to the category of signal transduction-related protein.

BRAMY3004800, BRAWH3009017, BRHIP2026877, BRTHA2013610, BRTHA2017972, BRTHA3003000, CTONG2020974, FEBRA2001990, FEBRA2008692, NT2RI2005772, NT2RI3007443, NTONG2008093, OCBBF2005433, OCBBF2024284, OCBBF2034637, OCBBF3002654, SPLEN2036702, SPLEN2039379, TESOP2000390, TESTI2025924, TESTI2049956, TESTI4000319, TESTI4019657, TESTI4021482, TESTI4024387, TESTI4025268, TESTI4031745, THYMU2004139, THYMU2031249, UTERU2008040, UTERU3000738

The clones predicted to belong to the category of transcription-related protein are the following 38 clones.

BRACE2030326, BRACE3001002, BRACE3045033, BRHIP3025161, BRSSN2014299, BRTHA2014792, BRTHA3001721, CTONG2025516, FEBRA2007544, FEBRA2007801, HEART1000074, IMR322000127, IMR322000917, NT2NE2006531, NT2RI2006686, NT2RI3009158, OCBBF2020838, OCBBF2036743, PEBLM2002887, SKNMC2007504, SPLEN2012624, TESTI2026505, TESTI2040018, TESTI2044796, TESTI2050987, TESTI4001176, TESTI4007810, TESTI4014175, TESTI4017543, TESTI4026524, TESTI4036909, THYMU2006420, THYMU2037233, THYMU3004866, TRACH3000558, TUTER2000425, UTERU2035328, UTERU3009490

The following 64 clones are also predicted to belong to the category of transcription-related protein.

BRACE2003609, BRACE3001058, BRACE3001113, BRALZ2017844, BRAMY2035070, BRAMY2035449, BRAMY2035718, BRAMY2039341, BRAWH2006207, BRHIP2017553, CERVX2002013, CTONG1000113, CTONG2003348, CTONG2008721, CTONG2020378, CTONG2020411, CTONG2028758, CTONG3004726, DFNES2011192, FCBBF3010361, FEBRA2014122, FEBRA2027609, HCASM2003018, HCHON2004858, HSYRA2005628, MESAN2005303, MESAN2014412, MESAN2015501, NT2RI2008952, NT2RI2018448, NT2RI3001132, OCBBF2008144, OCBBF2009583, OCBBF2011669, OCBBF2019684, OCBBF2032274, OCBBF3000167, SPLEN2004611, SPLEN2016135, SPLEN2016781, SYNOV2021953, SYNOV4002744, TESOP2001796, TESOP2005199, TESOP2006398, TESOP2006865, TESTI2034251, TESTI4000183, TESTI4000214, TESTI4008302, TESTI4015442, TESTI4025494, TESTI4025547, TESTI4028938, TESTI4032112, THYMU2006001, THYMU2028739, TRACH2007483, TRACH3000134, TRACH3003832, TUTER2000057, UTERU2033577, UTERU3001053, TESTI4038779

The clones predicted to belong to the category of disease-related protein are the following 342 clones.

3NB692002806, ADIPS2000088, BLADE2005036, BRACE2005457, BRACE2008594, BRACE2014306, BRACE2016981, BRACE2018762, BRACE2035381, BRACE2038551, BRACE2039249, BRACE2045300, BRACE3000840, BRACE3001002, BRACE3001391, BRACE3001754, BRACE3002508, BRACE3003595, BRACE3004058, BRACE3004150, BRACE3004772, BRACE3008137, BRACE3008384, BRACE3009708, BRACE3010397, BRACE3011271, BRACE3011421, BRACE3014807, BRACE3015027, BRACE3015521, BRACE3018963, BRACE3020594, BRACE3027326, BRALZ2017359, BRAMY2005052, BRAMY2038904, BRAMY2047751, BRAMY3000213, BRAMY3005091, BRAMY3007609, BRAMY4000095, BRAMY4000229, BRAMY4000277, BRAWH2001395, BRAWH2002560, BRAWH2010000, BRAWH2010536, BRAWH2014414, BRAWH3000100, BRAWH3000491, BRAWH3001326, BRAWH3002574, BRAWH3005912, BRAWH3008341, BRCAN2002562, BRCAN2002856, BRCAN2002948, BRCAN2003746, BRCAN2006063, BRCAN2009203, BRCAN2014602, BRCAN2016619, BRCAN2017442, BRCAN2024451, BRCOC2001505, BRCOC2003213, BRHIP2000819, BRHIP2001805, BRHIP2009414, BRHIP2024165, BRHIP2026288, BRHIP3000339, BRHIP3008405, BRHIP3009448, BRHIP3027137, BRHIP3027854, BRSSN2000684, BRSSN2004719, BRSSN2014424, BRSTN2001613, BRSTN2004987, BRSTN2008418, BRTHA2002608, BRTHA2003110, BRTHA2007122, BRTHA2007603, BRTHA2008527, BRTHA2012980, BRTHA2014792, BRTHA3001721, BRTHA3002427, BRTHA3003074, BRTHA3003449, BRTHA3008778, BRTHA3009037, BRTHA3009090, BRTHA3015815, BRTHA3016917, BRTHA3017848, COLON2000568, COLON2002520, CTONG1000341, CTONG2000042, CTONG2009423, CTONG2010803, CTONG2017500, CTONG2023021, CTONG2025496, CTONG2025516, CTONG3000084, CTONG3002412, CTONG3008639, D3OST2002182, D3OST2002648, DFNES2001108, FCBBF3009888, FEBRA2007708, FEBRA2008468, FEBRA2024744, HCASM2001301, HCASM2007737, HCHON2001712, HCHON2002676, HCHON2003532, HCHON2004007, HCHON2004531, HCHON2008112, HCHON2008444, HEART1000010, HEART1000139, HEART2001680, HEART2010495, HLUNG2002465, HSYRA2005496, IMR322000127, IMR322001380, IMR322006495, KIDNE2001847, KIDNE2012945, NESOP2001694, NOVAR2001108, NT2NE2003252, NT2NE2006531, NT2NE2006909, NT2RI2006686, NT2RI2025909, NT2RI3001515, NT2RI3006171, NT2RI3006340, NT2RI3006673, NT2RI3007757, NT2RI3008652, NT2RP7000359, NT2RP7005118, NT2RP7005529, NT2RP7010599, NTONG2000413, OCBBF2006058, OCBBF2020801, OCBBF2021788, OCBBF2031167, OCBBF2033869, OCBBF2036743, OCBBF2037068, OCBBF2037340, OCBBF3003320, PEBLM2000170, PEBLM2002887, PERIC2003720, PERIC2007914, PERIC2008385, PERIC2009086, PLACE5000282, PLACE6019385, PROST1000184, PROST2003428, PROST2016462, PROST2017367, PROST2018090, PROST2018511, PUAEN2002489, PUAEN2009795, SKNMC2007504, SMINT2010076, SPLEN2002467, SPLEN2006122, SPLEN2011422, SPLEN2012624, SPLEN2021701, SPLEN2031547, SPLEN2033098, SPLEN2036326, SPLEN2036821, SPLEN2036932, SYNOV2005817, SYNOV2012326, SYNOV2014400, SYNOV2021320, SYNOV3000231, SYNOV3000302, SYNOV4002883, SYNOV4004741, SYNOV4007360, SYNOV4007521, SYNOV4007553, SYNOV4007671, SYNOV4008440, TBAES2001229, TBAES2001258, TESOP2004114, TESOP2005485, TESOP2009121, TESTI1000257, TESTI1000319, TESTI2000644, TESTI2002618, TESTI2005610, TESTI2024567, TESTI2026505, TESTI2050987, TESTI2051867, TESTI2053399, TESTI2053621, TESTI4000014, TESTI4000079, TESTI4000288, TESTI4000349, TESTI4000724, TESTI4001148, TESTI4001176, TESTI4001527, TESTI4001561, TESTI4002491, TESTI4006420, TESTI4006819, TESTI4007163, TESTI4007778, TESTI4007810, TESTI4008050, TESTI4008429, TESTI4009160, TESTI4009457, TESTI4009881, TESTI4010851, TESTI4011745, TESTI4011956, TESTI4012406, TESTI4012448, TESTI4012505, TESTI4012679, TESTI4013369, TESTI4013924, TESTI4014175, TESTI4016110, TESTI4016822, TESTI4016925, TESTI4017901, TESTI4018835, TESTI4018881, TESTI4018886, TESTI4020092, TESTI4021478, TESTI4022873, TESTI4023546, TESTI4026524, TESTI4027557, TESTI4028059, TESTI4028429, TESTI4028880, TESTI4030069, TESTI4034632, TESTI4034912, TESTI4035063, TESTI4035498, TESTI4036909, TESTI4037156, TESTI4040363, THYMU1000496, THYMU2005303, THYMU2008725, THYMU2019210, THYMU2027497, THYMU2027695, THYMU2027734, THYMU2031046, THYMU2033104, THYMU2035319, THYMU2037233, THYMU2041015, THYMU3001083, THYMU3001234, THYMU3001379, THYMU3003309, THYMU3004835, THYMU3006118, THYMU3007137, THYMU3008436, TKIDN2000701, TKIDN2006852, TLIVE2001327, TRACH2001549, TRACH2007059, TRACH2022425, TRACH2022649, TRACH3000558, TRACH3002168, TRACH3004721, TRACH3004786, TRACH3005549, TRACH3007479, TRACH3008629, TRACH3009455, TRACH3035526, TSTOM2000442, TUTER2000904, UTERU1000337, UTERU2005621, UTERU2007724, UTERU2017762, UTERU2019491, UTERU2019706, UTERU2025025, UTERU2026090, UTERU2027591, UTERU2035328, UTERU3000645, UTERU3000828, UTERU3000899, UTERU3001240, UTERU3001572, UTERU3001585, UTERU3001652, UTERU3003116, UTERU3003135, UTERU3005907, UTERU3007640, UTERU3008671, UTERU3009490, UTERU3009690, UTERU3009979, UTERU3015500, UTERU3016789

The following 84 clones are also predicted to belong to the category of disease-related protein.

BRACE3001113, BRACE3010076, BRAMY2039341, BRAMY3004800, BRAWH3009017, BRCAN2002473, BRCAN2002854, BRCAN2003070, BRHIP2005271, BRHIP2017553, BRHIP2026877, BRHIP3000240, BRHIP3008314, BRHIP3026052, BRSTN2013354, BRTHA2016318, BRTHA2017972, BRTHA3003000, CERVX2002013, CTONG1000113, CTONG2008721, CTONG2020411, CTONG3004550, FCBBF1000509, FEBRA2008692, HCASM2008536, HCHON2004858, HEART2009680, HLUNG2015548, HSYRA2005628, IMR322008651, IMR322013396, MESAN2001770, NT2RI2009583, NT2RI3007443, OCBBF2003327, OCBBF2009583, OCBBF2011669, OCBBF2024284, OCBBF2032274, OCBBF3000167, OCBBF3002654, PLACE7000502, PROST2000452, PROST2009320, SPLEN2004611, STOMA2003158, SYNOV1000256, SYNOV4002744, SYNOV4003981, TBAES2000932, TESOP2000390, TESOP2001796, TESOP2005199, TESTI2015626, TESTI2025924, TESTI2026647, TESTI2039060, TESTI4000183, TESTI4006473, TESTI4011070, TESTI4017714, TESTI4019657, TESTI4021482, TESTI4024387, TESTI4025494, TESTI4025547, TESTI4028938, TESTI4031745, TESTI4032112, THYMU2004284, THYMU2028739, THYMU2031139, THYMU2031249, THYMU2035710, THYMU3000269, TLIVE2001684, TLIVE2002046, TRACH2024408, TRACH3003683, UTERU2021820, UTERU2032279, UTERU2033577, UTERU3000738

In particular, hit data of the following 338 clones for Swiss-Prot, or GenBank, UniGene, nr or RefSeq corresponded to genes or proteins which had been deposited in the Online Mendelian Inheritance in Man (OMIM), which is the human gene and disease database (the OMIM Number is shown in the parenthesis after the Clone Name).

3NB692002806 (261630), ADIPS2000088 (147120), BLADE2005036 (114850), BRACE2005457 (274600; 603545; 600791), BRACE2008594 (601959), BRACE2014306 (193002), BRACE2016981 (602701), BRACE2018762 (604800), BRACE2035381 (606088), BRACE2038551 (601961), BRACE2039249 (602273), BRACE2045300 (601442), BRACE3000840 (600355), BRACE3001002 (300236), BRACE3001391 (601313; 173900), BRACE3001754 (185641), BRACE3002508 (606417), BRACE3003595 (602941), BRACE3004058 (250800), BRACE3004150 (601035), BRACE3004772 (603143), BRACE3008137 (602187), BRACE3008384 (603264), BRACE3009708 (182340), BRACE3010397 (602187), BRACE3011271 (602187), BRACE3011421 (602187), BRACE3014807 (605784), BRACE3015027 (602187), BRACE3015521 (605888), BRACE3018963 (605744), BRACE3020594 (400023), BRACE3027326 (602187), BRALZ2017359 (604331), BRAMY2005052 (602621), BRAMY2038904 (605671), BRAMY2047751 (602512), BRAMY3000213 (605448), BRAMY3005091 (600286), BRAMY3007609 (300315), BRAMY4000095 (602187), BRAMY4000229 (602159), BRAMY4000277 (602187), BRAWH2001395 (159430), BRAWH2002560 (602865), BRAWH2010000 (602581), BRAWH2010536 (604010), BRAWH2014414 (603006), BRAWH3000100 (601403), BRAWH3000491 (602187), BRAWH3001326 (602187), BRAWH3002574 (602187), BRAWH3005912 (602187), BRAWH3008341 (602187), BRCAN2002562 (602187), BRCAN2002856 (602712), BRCAN2002948 (603534), BRCAN2003746 (311870), BRCAN2006063 (603196; 601369), BRCAN2009203 (603143), BRCAN2014602 (601441), BRCAN2016619 (602187), BRCAN2017442 (604455), BRCAN2024451 (602513), BRCOC2001505 (159430), BRCOC2003213 (602187), BRHIP2000819 (605000), BRHIP2001805 (603219), BRHIP2009414 (602187), BRHIP2024165 (604402), BRHIP2026288 (602187), BRHIP3000339 (159430), BRHIP3008405 (602187), BRHIP3009448 (602187), BRHIP3027137 (600249), BRHIP3027854 (601060), BRSSN2000684 (603505), BRSSN2004719 (600560), BRSSN2014424 (606105), BRSTN2001613 (164020), BRSTN2004987 (604733), BRSTN2008418 (602187), BRTHA2002608 (600463), BRTHA2003110 (602187), BRTHA2007122 (106410), BRTHA2007603 (605846), BRTHA2008527 (152790; 176410), BRTHA2012980 (300119), BRTHA2014792 (601674), BRTHA3001721 (604902), BRTHA3002427 (602187), BRTHA3003074 (605367), BRTHA3003449 (160745), BRTHA3008778 (602187), BRTHA3009037 (602187), BRTHA3009090 (603197), BRTHA3015815 (600902), BRTHA3016917 (604137), BRTHA3017848 (603377; 212140), COLON2000568 (147000), COLON2002520 (602187), CTONG1000341 (188040), CTONG2000042 (103950), CTONG2009423 (182137), CTONG2010803 (602189), CTONG2023021 (602498), CTONG2025496 (103950), CTONG2025516 (601679), CTONG3000084 (600888), CTONG3002412 (601403), CTONG3008639 (601797), D3OST2002182 (603590), D3OST2002648 (603071), DFNES2001108 (603560), FCBBF3009888 (602470), FEBRA2007708 (126650; 214700), FEBRA2008468 (278000), HCASM2001301 (602399), HCASM2007737 (601504), HCHON2001712 (109190), HCHON2002676 (252800), HCHON2003532 (172490), HCHON2004007 (605866), HCHON2004531 (602187), HCHON2008112 (605837), HCHON2008444 (602187), HEART1000010 (602187), HEART1000139 (191045; 115195), HEART2001680 (146900), HEART2010495 (157132), HLUNG2002465 (605216), HSYRA2005496 (131195; 187300), IMR322000127 (604077), IMR322001380 (605652), IMR322006495 (605607), KIDNE2012945 (600270), NOVAR2001108 (147120), NT2NE2003252 (602913), NT2NE2006531 (602277), NT2NE2006909 (602187), NT2RI2006686 (602700), NT2RI2025909 (212138), NT2RI3001515 (300362), NT2RI3006171 (114890), NT2RI3006340 (602187), NT2RI3006673 (602187), NT2RI3007757 (605396), NT2RI3008652 (602654), NT2RP7000359 (603271), NT2RP7005118 (603379), NT2RP7005529 (600888), NT2RP7010599 (603684), NTONG2000413 (602262), OCBBF2006058 (604773), OCBBF2020801 (602187), OCBBF2021788 (602597), OCBBF2031167 (603709), OCBBF2033869 (600270), OCBBF2036743 (604075), OCBBF2037068 (602187), OCBBF2037340 (602187), OCBBF3003320 (605868), PEBLM2000170 (602187), PEBLM2002887 (602187), PERIC2003720 (600381), PERIC2007914 (400009), PERIC2008385 (604455), PERIC2009086 (600134; 605158), PLACE5000282 (130160), PLACE6019385 (602448), PROST1000184 (192321), PROST2003428 (602187), PROST2016462 (602187), PROST2017367 (600585), PROST2018090 (312610), PROST2018511 (602187), PUAEN2002489 (604658), PUAEN2009795 (601456), SKNMC2007504 (602187), SMINT2010076 (146900), SPLEN2002467 (605652), SPLEN2006122 (604739), SPLEN2011422 (114213), SPLEN2012624 (602187), SPLEN2021701 (142800), SPLEN2031547 (602187), SPLEN2033098 (602746), SPLEN2036326 (602101), SPLEN2036821 (212138), SPLEN2036932 (605577), SYNOV2005817 (123889), SYNOV2012326 (604336), SYNOV2014400 (135820), SYNOV2021320 (602104), SYNOV3000231 (147100), SYNOV3000302 (147100), SYNOV4002883 (602187), SYNOV4004741 (602187), SYNOV4007360 (602187), SYNOV4007521 (605830), SYNOV4007553 (603028), SYNOV4007671 (602187), SYNOV4008440 (602187), TBAES2001229 (602187), TBAES2001258 (142440), TESOP2004114 (601865), TESOP2005485 (147170), TESOP2009121 (117143), TESTI1000257 (138170), TESTI1000319 (602187), TESTI2000644 (601392), TESTI2002618 (601533), TESTI2005610 (601040), TESTI2024567 (601116), TESTI2026505 (305400), TESTI2050987 (605968), TESTI2051867 (180479), TESTI2053399 (605819), TESTI2053621 (600364; 602093), TESTI4000014 (602187), TESTI4000079 (603560), TESTI4000288 (602187), TESTI4000349 (604506), TESTI4000724 (603878), TESTI4001148 (602187), TESTI4001176 (601430), TESTI4001527 (602187), TESTI4001561 (602187), TESTI4002491 (602187), TESTI4006420 (605612), TESTI4006819 (602187), TESTI4007163 (602187), TESTI4007778 (602187), TESTI4007810 (600940), TESTI4008050 (602187), TESTI4008429 (602187), TESTI4009160 (602187), TESTI4009457 (606185), TESTI4009881 (602187), TESTI4010851 (602187), TESTI4011745 (602187), TESTI4011956 (602187), TESTI4012406 (602187), TESTI4012448 (185261), TESTI4012505 (602143), TESTI4012679 (601933), TESTI4013369 (602187), TESTI4013924 (602187), TESTI4014175 (602187), TESTI4016110 (602187), TESTI4016822 (601792), TESTI4016925 (602187), TESTI4017901 (104221), TESTI4018835 (602187), TESTI4018881 (605070), TESTI4018886 (602187), TESTI4020092 (156225), TESTI4021478 (605868), TESTI4022873 (602187), TESTI4023546 (602187), TESTI4026524 (603277), TESTI4027557 (602187), TESTI4028059 (232800; 171850), TESTI4028429 (602187), TESTI4028880 (138170), TESTI4030069 (604603), TESTI4034632 (606251), TESTI4034912 (602187), TESTI4035063 (602187), TESTI4035498 (602187), TESTI4036909 (602187), TESTI4037156 (606026), TESTI4040363 (185641), THYMU1000496 (603060), THYMU2005303 (186910), THYMU2008725 (176882), THYMU2019210 (142830), THYMU2027497 (182139), THYMU2027695 (147100), THYMU2027734 (145505), THYMU2031046 (604207), THYMU2033104 (605349), THYMU2035319 (604739), THYMU2037233 (605121), THYMU2041015 (602187), THYMU3001083 (602187), THYMU3001234 (602187), THYMU3001379 (602187), THYMU3003309 (300359), THYMU3004835 (602187), THYMU3006118 (603708), THYMU3007137 (602187), THYMU3008436 (602187), TKIDN2000701 (600465), TKIDN2006852 (603602), TLIVE2001327 (601403), TRACH2001549 (603197), TRACH2007059 (602187), TRACH2022425 (146900), TRACH2022649 (147100), TRACH3000558 (600140), TRACH3002168 (155735), TRACH3004721 (602187), TRACH3004786 (602187), TRACH3005549 (602187), TRACH3007479 (602308), TRACH3008629 (600976), TRACH3009455 (171833), TRACH3035526 (147000), TSTOM2000442 (147100), TUTER2000904 (602187), UTERU1000337 (602187), UTERU2005621 (603505), UTERU2007724 (602373), UTERU2017762 (601053), UTERU2019491 (603762), UTERU2019706 (600114), UTERU2025025 (191315; 164970; 256000), UTERU2026090 (605497), UTERU2027591 (600150), UTERU2035328 (605409), UTERU3000645 (602909), UTERU3000828 (602187), UTERU3000899 (603062), UTERU3001240 (602187), UTERU3001572 (602187), UTERU3001585 (602187), UTERU3001652 (602715), UTERU3003116 (602187), UTERU3003135 (602187), UTERU3005907 (190196), UTERU3007640 (603215), UTERU3008671 (182120), UTERU3009490 (604585), UTERU3009690 (104221), UTERU3009979 (600441), UTERU3015500 (606667), UTERU3016789 (602104)

Additionally, hit data of the following 84 clones for Swiss-Prot, nr or RefSeq corresponded to genes or proteins which had been deposited in the Online Mendelian Inheritance in Man (OMIM), which is the human gene and disease database (the OMIM Number is shown in the parenthesis after the Clone Name).

BRACE3001113 (603971), BRACE3010076 (142695), BRAMY2039341 (604077), BRAMY3004800 (602187), BRAWH3009017 (602187), BRCAN2002473 (602187), BRCAN2002854 (602895), BRCAN2003070 (605574), BRHIP2005271 (600267), BRHIP2017553 (602187), BRHIP2026877 (600341), BRHIP3000240 (601142), BRHIP3008314 (604480), BRHIP3026052 (601645), BRSTN2013354 (602187), BRTHA2016318 (605442), BRTHA2017972 (602932), BRTHA3003000 (605276), CERVX2002013 (602903), CTONG1000113 (602277), CTONG2008721 (605317), CTONG2020411 (601930), CTONG3004550 (605611), FCBBF1000509 (601933), FEBRA2008692 (604034), HCASM2008536 (194360), HCHON2004858 (602187), HEART2009680 (601970), HLUNG2015548 (146690), HSYRA2005628 (602187), IMR322008651 (179617), IMR322013396 (600053), MESAN2001770 (600495), NT2RI2009583 (605949), NT2RI3007443 (602448), OCBBF2003327 (605008), OCBBF2009583 (602277), OCBBF2011669 (602187), OCBBF2024284 (176981), OCBBF2032274 (603975), OCBBF3000167 (194558), OCBBF3002654 (601893), PLACE7000502 (164951), PROST2000452 (602060), PROST2009320 (605903), SPLEN2004611 (602228), STOMA2003158 (602244), SYNOV1000256 (606021), SYNOV4002744 (602187), SYNOV4003981 (604283), TBAES2000932 (606212), TESOP2000390 (602187), TESOP2001796 (602187), TESOP2005199 (194531), TESTI2015626 (601249), TESTI2025924 (600863), TESTI2026647 (601235), TESTI2039060 (154360), TESTI4000183 (601276), TESTI4006473 (602187), TESTI4011070 (602187), TESTI4017714 (602187), TESTI4019657 (602052), TESTI4021482 (164730), TESTI4024387 (602187), TESTI4025494 (602187), TESTI4025547 (605308), TESTI4028938 (603899), TESTI4031745 (602448), TESTI4032112 (603246), THYMU2004284 (314370), THYMU2028739 (604191), THYMU2031139 (605009), THYMU2031249 (311550), THYMU2035710 (601890), THYMU3000269 (600857), TLIVE2001684 (120700), TLIVE2002046 (125270), TRACH2024408 (106410), TRACH3003683 (150205), UTERU2021820 (126141), UTERU2032279 (600942), UTERU2033577 (603397), UTERU3000738 (602187)

The clones predicted to belong to the category of enzyme and/or metabolism-related protein are the following 171 clones.

3NB692002806, ASTRO2002842, BLADE2005036, BRACE2008594, BRACE2030341, BRACE2035381, BRACE2038551, BRACE2039249, BRACE2041200, BRACE2045772, BRACE3004058, BRACE3009708, BRACE3011421, BRACE3016884, BRACE3024073, BRACE3025630, BRAMY2033267, BRAMY2039872, BRAMY3002803, BRAMY3004919, BRAMY3005091, BRAMY3005932, BRAMY4000095, BRAWH3002574, BRAWH3008341, BRCAN2003703, BRCAN2003746, BRCAN2009432, BRCAN2014602, BRCAN2017442, BRCAN2028355, BRCOC2003213, BRHIP2024165, BRHIP3008405, BRHIP3027137, BRHIP3027854, BRSTN2000872, BRSTN2004863, BRSTN2004987, BRSTN2008418, BRTHA2002608, BRTHA2009311, BRTHA2015406, BRTHA2016496, BRTHA3008778, BRTHA3009090, BRTHA3015815, BRTHA3016917, CTONG2004062, CTONG2006798, CTONG2013178, CTONG2028124, CTONG3009028, D3OST2002182, DFNES2001108, DFNES2005266, FCBBF3013307, FCBBF3023895, FEBRA2008468, FEBRA2026984, HCASM2001301, HCHON2002676, HCHON2003532, HCHON2004007, HEART2006131, HEART2010492, HHDPC1000118, HLUNG2011298, HLUNG2013204, HSYRA2008714, KIDNE2001361, KIDNE2006580, NT2NE2003252, NT2NE2006909, NT2RI2004618, NT2RI2025909, NT2RI3006673, NT2RI3007978, NT2RI3008974, NT2RP7000359, NT2RP7004027, NT2RP7010599, NT2RP7014005, NTONG2000413, NTONG2008672, OCBBF2006005, OCBBF2006058, OCBBF2006151, OCBBF2019823, OCBBF2025527, OCBBF2030354, OCBBF2031167, OCBBF3003320, PEBLM2005183, PERIC2000889, PERIC2008385, PLACE6019385, PLACE7008431, PROST2017367, PUAEN2007044, PUAEN2009655, PUAEN2009852, SKNMC2006998, SKNMC2007504, SMINT1000192, SPLEN2010912, SYNOV2012326, SYNOV4002883, TBAES2001258, TESOP2000801, TESOP2004114, TESTI2005610, TESTI2005739, TESTI2016046, TESTI4000079, TESTI4000209, TESTI4000288, TESTI4000349, TESTI4001176, TESTI4001527, TESTI4001561, TESTI4002552, TESTI4006148, TESTI4006819, TESTI4007810, TESTI4008429, TESTI4010851, TESTI4012406, TESTI4012448, TESTI4013369, TESTI4013817, TESTI4014175, TESTI4016822, TESTI4018152, TESTI4018835, TESTI4019566, TESTI4021478, TESTI4022716, TESTI4023546, TESTI4026510, TESTI4026524, TESTI4028059, TESTI4029836, TESTI4034632, TESTI4036909, TESTI4046819, THYMU2008725, THYMU2027734, THYMU2031046, THYMU2031258, THYMU3001234, THYMU3003212, THYMU3004157, THYMU3004835, THYMU3006118, THYMU3008436, TKIDN2006852, TLIVE2002336, TRACH2001549, TRACH2009310, TRACH3007479, TRACH3036193, UTERU1000337, UTERU2019491, UTERU2025025, UTERU2026203, UTERU3000665, UTERU3001240, UTERU3001585, UTERU3003116, UTERU3005907

The following 59 clones are also predicted to belong to the category of enzyme and/or metabolism-related protein.

BRACE2039823, BRACE3010076, BRAMY2038516, BRAWH1000369, BRCAN2003070, BRHIP2005271, BRHIP2012360, BRHIP2026877, BRHIP3008314, BRTHA2013610, BRTHA2017364, BRTHA2017972, BRTHA2018011, BRTHA2018443, BRTHA3003000, CTONG2016942, FCBBF1000509, FEBRA2008692, HCASM2003099, HLUNG2015548, MESAN2005303, NT2RI3000174, NT2RI3007443, NT2RP7008435, NTONG2008093, OCBBF2003327, OCBBF2034637, OCBBF3002654, PROST2000452, SPLEN2039311, SPLEN2039379, STOMA2003158, TESOP2000390, TESTI2015626, TESTI2025924, TESTI2026647, TESTI2032643, TESTI2036288, TESTI2039060, TESTI4006473, TESTI4011070, TESTI4014801, TESTI4017714, TESTI4019657, TESTI4021482, TESTI4031745, THYMU2004139, THYMU2004284, THYMU2031139, THYMU2031249, THYMU2040925, THYMU3000269, TLIVE2002046, TLIVE2007607, TRACH2024559, TRACH3003683, TRACH3007866, UTERU2021820, UTERU3000738

The clones predicted to belong to the category of cell division and/or cell proliferation-related protein are the following 42 clones.

BLADE2002782, BRACE2042550, BRACE2043248, BRACE3000840, BRALZ2017359, BRAMY2038484, BRAMY2046989, BRAWH2010536, BRAWH2014954, BRAWH3000100, BRHIP2000819, BRHIP2001927, BRHIP2009414, BRSSN2000684, CTONG3002412, CTONG3008258, CTONG3008639, FCBBF3002163, HCASM2001301, IMR322006495, NT2RI2006686, OCBBF2021020, OCBBF2037068, OCBBF3004314, PLACE5000282, PLACE6019385, PLACE7002641, PUAEN2006328, SPLEN2033098, TESOP2009121, TESTI1000545, TESTI2003573, TESTI2005610, TESTI4007810, TESTI4017901, THYMU2034374, THYMU2039315, TLIVE2001327, TRACH2025507, UTERU2005621, UTERU3009690, UTERU3009979

The following ten clones are also predicted to belong to the category of cell division and/or cell proliferation-related protein.

BRCAN2003070, BRTHA3003000, NT2RI3007443, PLACE7000502, SPLEN2004611, STOMA2003158, SYNOV4003981, TESTI4031745, THYMU2004139, THYMU2035078

The clones predicted to belong to the category of cytoskeleton-related protein are the following 55 clones.

ASTRO1000009, BLADE2004089, BRACE2026836, BRACE2045300, BRACE3006872, BRAMY3008466, BRAWH2001395, BRAWH2005315, BRAWH3002600, BRCOC2001505, BRHIP2000819, BRHIP3000339, BRHIP3008405, BRTHA2007122, BRTHA3003449, COLON2002520, CORDB2000541, FCBBF3021940, HCHON2001577, HEART1000139, HEART2010495, NT2RI3006340, NT2RP7000359, NTONG2005277, OCBBF2007068, OCBBF3003592, PERIC2000889, PLACE5000282, PROST1000559, SKMUS2006394, SPLEN2011422, SPLEN2015679, TESTI2049857, TESTI4000288, TESTI4001148, TESTI4007778, TESTI4009160, TESTI4009881, TESTI4011956, TESTI4013924, TESTI4016925, TESTI4018886, TESTI4022873, TESTI4034912, TESTI4035063, TESTI4037727, THYMU1000496, THYMU2035735, THYMU3001083, THYMU3001234, TKIDN2000701, UTERU2007724, UTERU2008347, UTERU2035745, UTERU3003178

The following six clones are also predicted to belong to the category of cytoskeleton-related protein.

HLUNG2015418, SPLEN2030847, SPLEN2036702, TESTI4025268, TESTI4026207, TRACH2024408

The clones predicted to belong to the category of nuclear protein and/or RNA synthesis-related protein are the following 42 clones.

BLADE2007958, BRACE2010489, BRACE2045300, BRACE3004150, BRACE3005430, BRACE3011421, BRAMY2046989, BRAMY3005932, BRCAN2002562, BRHIP2021615, BRSTN2001613, BRSTN2004987, COLON2000470, CTONG3009028, FCBBF3013307, HCHON2004531, IMR322006495, OCBBF2020801, PEBLM2005183, PUAEN2007044, SKNMC1000124, SMINT1000192, SPLEN2006122, SPLEN2010912, TESOP2009121, TESTI4009374, TESTI4009457, TESTI4013830, TESTI4019566, TESTI4022716, THYMU2033104, THYMU2035319, THYMU2038301, THYMU2040975, THYMU3001379, TRACH3004721, TRACH3036609, UTERU2026025, UTERU3000828, UTERU3001572, UTERU3003135, UTERU3004992

The following 16 clones are also predicted to belong to the category of nuclear protein and/or RNA synthesis-related protein.

BRACE3010076, BRCAN2002854, BRHIP2006617, BRHIP2012360, BRHIP3026052, BRSTN2013354, BRTHA2017364, HCASM2003099, HCASM2008536, IMR322008651, NT2RI3000174, STOMA2003158, TESTI2026647, TESTI4006473, TESTI4021482, THYMU2035078

The clones predicted to belong to the category of protein synthesis and/or transport-related protein are the following 57 clones.

ASTRO2002842, BLADE2005036, BRACE3025630, BRAMY2033003, BRAMY3007609, BRAWH3000491, BRAWH3002574, BRAWH3008341, BRCAN2002856, BRCAN2002948, BRCOC2003213, BRSTN2004987, BRTHA2016496, BRTHA3013884, BRTHA3016917, CTONG2000042, CTONG2013178, CTONG2023512, CTONG2024749, CTONG2025496, CTONG3001370, DFNES2005266, FEBRA2026984, HCASM2007737, HCHON2008444, HEART1000010, KIDNE2000846, NT2NE2006909, NT2RI2011422, NT2RP7004027, NTONG2000413, OCBBF2031167, TBAES2001229, TBAES2001258, TESTI1000319, TESTI2005610, TESTI2051867, TESTI4000209, TESTI4000349, TESTI4001106, TESTI4002491, TESTI4008050, TESTI4010851, TESTI4012406, TESTI4012448, TESTI4013924, TESTI4028429, TESTI4034912, THYMU2009157, TLIVE2008229, TRACH3007479, TRACH3008713, TRACH3036193, UTERU2019940, UTERU3001988, UTERU3003116, UTERU3007419

The following 15 clones are also predicted to belong to the category of protein synthesis and/or transport-related protein.

BRTHA2007060, BRTHA2018011, CTONG2016942, MESAN2001770, MESAN2005303, NT2RP7008435, OCBBF2003327, PROST2000452, TESOP2001796, TESTI4017714, THYMU2004284, THYMU2031139, TRACH2024559, TRACH3007866, UTERU2021820

The clones predicted to belong to the category of cellular defense-related protein are the following three clones.

BRACE3005430, HCHON2004531, TESTI4007810

The following four clones are also predicted to belong to the category of cellular defense-related protein.

BRHIP2012360, FCBBF3027854, HCASM2008536, UTERU2032279

The clones predicted to belong to the category of development and/or differentiation-related protein are the following nine clones.

BRACE3009747, BRTHA2005579, BRTHA3003343, IMR322000917, PEBLM2000170, TESOP2001122, TESOP2001953, TESTI2040018, UTERU3006308

The following five clones are also predicted to belong to the category of development and/or differentiation-related protein.

BRALZ2017844, CTONG2020378, HCHON2004858, OCBBF2019684, THYMU2006001

The clones predicted to belong to the category of DNA-binding and/or RNA-binding protein are the following 55 clones.

3NB692002685, BLADE2007958, BRACE2030326, BRACE2045596, BRACE3001002, BRACE3004150, BRACE3009747, BRACE3045033, BRCAN2002562, BRHIP2021615, BRSSN2014299, BRSTN2001613, BRSTN2004987, BRTHA2014792, BRTHA3001721, BRTHA3003343, CTONG2025516, CTONG3008831, CTONG3009028, FCBBF3013307, FEBRA2007544, FEBRA2007801, HEART1000074, IMR322000127, IMR322000917, NT2NE2006531, NT2RI3009158, OCBBF2020838, OCBBF2036743, PEBLM2002887, PEBLM2005183, SKNMC2007504, SMINT1000192, SPLEN2006122, TBAES2001229, TESTI2014716, TESTI2040018, TESTI2044796, TESTI4009374, TESTI4012679, TESTI4014175, TESTI4017543, TESTI4026510, TESTI4026524, THYMU2006420, THYMU2035319, THYMU2037233, THYMU2040975, THYMU3004866, TLIVE2008229, TRACH3036609, TUTER2000425, UTERU2026025, UTERU2035328, UTERU3009490

The following 74 clones are also predicted to belong to the category of DNA-binding and/or RNA-binding protein.

BRACE2003609, BRACE3001058, BRACE3001113, BRACE3010076, BRALZ2017844, BRAMY2035070, BRAMY2035449, BRAMY2035718, BRAMY2039341, BRAWH1000369, BRAWH2006207, BRCAN2002854, BRHIP2012360, BRHIP2017553, BRSTN2013354, BRTHA2017364, CERVX2002013, CTONG1000113, CTONG2008721, CTONG2020378, CTONG2020411, CTONG2028758, CTONG3004726, DFNES2011192, FEBRA2014122, FEBRA2027609, HCASM2003018, HCASM2009424, HCHON2004858, HSYRA2005628, IMR322008651, MESAN2001770, MESAN2005303, MESAN2014412, MESAN2015501, NT2RI2008952, NT2RI2018448, NT2RI3000174, NT2RI3001132, OCBBF2008144, OCBBF2009583, OCBBF2011669, OCBBF2019684, OCBBF2032274, OCBBF3000167, SPLEN2004611, SPLEN2016135, SPLEN2016781, SYNOV2021953, SYNOV4002744, TESOP2005199, TESOP2006398, TESOP2006865, TESTI2026647, TESTI2034251, TESTI4000183, TESTI4000214, TESTI4006473, TESTI4008302, TESTI4015442, TESTI4025494, TESTI4025547, TESTI4028938, TESTI4032112, THYMU2006001, THYMU2035078, TRACH2007483, TRACH3000134, TRACH3002561, TRACH3003832, TUTER2000057, UTERU2033577, UTERU3001053, TESTI4038779

The clones predicted to belong to the category of ATP binding and/or GTP-binding protein are the following 68 clones.

BNGH42007788, BRACE2008594, BRACE2047377, BRACE3005430, BRACE3008720, BRACE3009708, BRACE3015521, BRACE3024073, BRAMY4000095, BRCAN2009432, BRCOC2003213, BRHIP3008405, BRSTN2013741, BRTHA3003449, BRTHA3015815, BRTHA3016917, COLON2002520, FEBRA2026984, HCASM2001301, HCHON2004007, HSYRA2008714, KIDNE2001361, KIDNE2001847, NESOP2001694, NT2RI2005166, NT2RP7013795, OCBBF3003320, OCBBF3003592, PEBLM2002594, PERIC2000889, PLACE6019385, SMINT1000192, SPLEN2037194, TESOP2000801, TESTI2006648, TESTI4000288, TESTI4001148, TESTI4001176, TESTI4002552, TESTI4007810, TESTI4008429, TESTI4009160, TESTI4009881, TESTI4011956, TESTI4013817, TESTI4014175, TESTI4016925, TESTI4018208, TESTI4018835, TESTI4019566, TESTI4021478, TESTI4022873, TESTI4026524, TESTI4029836, TESTI4035498, TESTI4036909, TESTI4037727, THYMU1000496, THYMU2033079, THYMU3001083, THYMU3001234, THYMU3001379, TRACH2009310, UTERU2019706, UTERU2025025, UTERU2035745, UTERU3000665, UTERU3000828

The following 24 clones are also predicted to belong to the category of ATP binding and/or GTP-binding protein.

BRHIP2026877, BRTHA2017364, BRTHA2018443, IMR322008651, IMR322013731, NT2RI3007443, NTONG2008093, OCBBF3002654, TESOP2000390, TESOP2007384, TESTI2025924, TESTI2026647, TESTI2049956, TESTI4005317, TESTI4006473, TESTI4021482, TESTI4026207, TESTI4031745, THYMU2004139, THYMU2031249, TRACH2000862, TRACH2024559, TRACH3000420, UTERU3000738

The 119 clones shown below are clones which were unassignable to any of the above-mentioned categories, but have been predicted to have some function based on homology search using their full-length nucleotide sequences. Clone Name and Definition in the result of homology search, demarcated by a double slash mark (//), are shown below.

ADRGL2009691//Mus musculus D11lgp1 mRNA, complete cds.

ADRGL2009755//Homo sapiens brain and reproductive organ-expressed protein (BRE) mRNA, complete cds.

ASTRO3000177//Drosophila melanogaster BcDNA.GH03694 (BcDNA.GH03694) mRNA, complete cds.

BLADE2008398//Homo sapiens LRR FLI-I interacting protein 2 (LRRFIP2) mRNA, complete cds.

BRACE2006319//Homo sapiens mRNA for Fln29, complete cds.

BRACE2027258//Homo sapiens E2a-Pbx1-associated protein (EB-1) mRNA, partial cds.

BRACE2038329//Rattus norvegicus CBL-B (Cbl-b) mRNA, partial cds.

BRACE2046251//Homo sapiens hucep-10 mRNA for cerebral protein-10, complete cds.

BRACE3003192//latent transforming growth factor beta binding protein 3 [Homo sapiens]

BRACE3007625//espin [Rattus norvegicus]

BRACE3009297//mdgl-1 [Mus musculus]

BRACE3015262//espin [Mus musculus]

BRACE3025457//testis-specific protein TSP-NY [Homo sapiens]

BRALZ2016498//Homo sapiens FKSG76 (FKSG76) mRNA, complete cds.

BRAMY2030109//Homo sapiens hucep-4 mRNA for cerebral protein-4, complete cds.

BRAMY2031317//Mus musculus semaphorin cytoplasmic domain-associated protein 3A (Semcap3) mRNA, complete cds.

BRAMY2047746//nasopharyngeal carcinoma susceptibility protein [Homo sapiens]

BRAMY3001794//Rattus norvegicus Circadian Oscillatory Protein (SCOP) (Scop)

BRAWH2001940//H. sapiens gene from PAC 1026E2, partial.

BRAWH2012162//KE03 protein [Homo sapiens]

BRAWH2016724//MAP2=HMW-MAP2 {alternatively spliced} [rats, brain, mRNA Partial, 267 nt].

BRAWH3002821//synaptotagmin-like 2 [Mus musculus]

BRCAN2002944//Mus musculus huntington yeast partner C (Hypc) mRNA, complete cds.

BRCAN2013660//Arabidopsis thaliana putative protein (F4F15.330) mRNA, complete cds.

BRHIP2002122//Homo sapiens B aggressive lymphoma long isoform (BAL) mRNA, complete cds.

BRHIP2003786//CCA3 [Rattus norvegicus]

BRHIP2004359//ELAC PROTEIN.

BRHIP2007616//plexin 2

BRHIP2029393//COBW-like protein [Homo sapiens]

BRHIP3008313//testis specific ankyrin-like protein 1 [Homo sapiens]

BRSSN2013874//TEMO [Rattus norvegicus]

BRSTN2017771//Homo sapiens putative BTK-binding protein mRNA, complete cds.

BRTHA2012392//Homo sapiens HCDI (HCDI) mRNA, complete cds.

BRTHA3002933//uroplakin 3 [Homo sapiens]

BRTHA3008310//Mus musculus mRNA for iroquois homeobox protein 6 (Irx6 gene).

BRTHA3008520//sporulation-induced transcript 4-associated protein; hypothetical protein FLJ11058 [Homo sapiens]

COLON2001721//GLUT4 vesicle protein [Mus musculus]

CTONG1000467//Mus musculus mRNA for Deltex3, complete cds.

CTONG2020026//Drosophila melanogaster BcDNA.GH09358 (BcDNA.GH09358) mRNA, complete cds.

CTONG3001123//Mus musculus Pax transcription activation domain interacting protein PTIP mRNA, complete cds.

CTONG3002127//granuphilin [Mus musculus]

CTONG3004072//GL002 protein [Homo sapiens]

CTONG3006186//syntaxin binding protein 4 [Mus musculus]

CTONG3008894//Mus musculus SH3-domain binding protein 5

FCBBF1000297//Human protein immuno-reactive with anti-PTH polyclonal antibodies mRNA, partial cds.

HCHON2000028//Homo sapiens 7h3 protein mRNA, partial cds.

HCHON2000626//X-linked protein STS1769.

HCHON2001217//Homo sapiens cullin CUL4B (CUL4B) mRNA, complete cds.

HEART2006909//Hemolysin C.

HLUNG2011041//basic proline-rich peptide IB-8a—human (fragments)

HLUNG2014288//Mus musculus RP42 mRNA, complete cds.

IMR322006886//Homo sapiens hepatocellular carcinoma-associated antigen 127 (HCA127) mRNA, complete cds.

KIDNE2002252//Drosophila melanogaster BcDNA.GH09358 (BcDNA.GH09358) mRNA, complete cds.

KIDNE2011532//similar to melanoma-associated chondroitin sulfate proteoglycan 4

NT2RI2012990//76.5 KDA PROTEIN C21ORF13.

NT2RI2025957//LU1 protein [Homo sapiens]

NT2RI3006284//Homo sapiens chorea-acanthocytosis (CHAC) mRNA, complete cds.

NT2RI3008697//erythroblast macrophage protein [Mus musculus]

NT2RP8000296//similar to Kelch proteins

NTONG2007517//RING CANAL PROTEIN (KELCH PROTEIN).

OCBBF2002124//p40 [Homo sapiens]

OCBBF2007610//PSD-95/SAP90-associated protein-4 [Rattus norvegicus].

OCBBF2021323//Mus musculus GTRGEO22 (Gtrgeo22) mRNA, complete cds.

OCBBF2028173//JM11 protein [Homo sapiens]

PEBLM2001465//diphthamide biosynthesis; Dph5p [Saccharomyces cerevisiae]

PERIC2004028//Mus musculus erythroblast macrophage protein EMP mRNA, complete cds.

PLACE7006051//cytoplasmic dynein heavy chain 2 [Rattus norvegicus]

PROST2008993//Mus musculus Pax transcription activation domain interacting protein PTIP mRNA, complete cds.

PUAEN2003079//nasopharyngeal carcinoma susceptibility protein [Homo sapiens]

SPLEN2002147//Halocynthia roretzi mRNA for HrPET-3, complete cds.

SPLEN2032154//NDRG1 PROTEIN (DIFFERENTIATION-RELATED GENE 1 PROTEIN) (DRG1) (REDUCING AGENTS AND TUNICAMYCIN-RESPONSIVE PROTEIN) (RTP) (NICKEL-SPECIFIC INDUCTION PROTEIN CAP43).

SYNOV2005216//Homo sapiens laryngeal carcinoma related protein 1 mRNA, complete cds.

SYNOV2007965//Homo sapiens mRNA for H-1(3)mbt-like protein, alternative variant a.

SYNOV4000706//B cell phosphoinositide 3-kinase adaptor [Mus musculus]

TBAES2004055//NY-REN-50 antigen

TESOP2001605//Homo sapiens laryngeal carcinoma related protein 1 mRNA, complete cds.

TESOP2005285//Homo sapiens partial mRNA for chr2 synaptotagmin (CHR2SYT gene).

TESTI2004215//Maackia amurensis early nodulin (ENOD2) mRNA, partial cds.

TESTI2009477//TRICHOHYALIN.

TESTI2034520//Rattus norvegicus SMC (segregation of mitotic chromosomes 1)-like 1 (yeast) (Smc1l1), mRNA

TESTI2052693//brk kinase substrate [Homo sapiens].

TESTI4006079//MUF1 protein; likely ortholog of mouse MUF1; elongin BC-interacting leucine-rich repeat protein [Homo sapiens]

TESTI4006393//neural specific sr protein NSSR 2 [Mus musculus]

TESTI4006546//colon cancer antigen NY-CO-45 [Homo sapiens].

TESTI4006802//mesothelin; megakaryocyte potentiating factor [Mus musculus]

TESTI4008018//DAZ associated protein 2; KIAA0058 gene product [Homo sapiens]

TESTI4009286//Homo sapiens HOTTL protein mRNA, complete cds

TESTI4009563//testis specific ankyrin-like protein 1 [Homo sapiens]

TESTI4010831//yeast Sec31p homolog; ABP125 [Homo sapiens]

TESTI4011484//Sec23-interacting protein p125 [Homo sapiens]

TESTI4014818//AD-012 protein [Homo sapiens]

TESTI4014924//selective hybridizing clone [Mus musculus]

TESTI4019140//Mi-2 histone deacetylase complex protein 66 [Xenopus laevis]

TESTI4019843//Rattus norvegicus huntingtin-associated protein interacting protein (duo) (Hapip), mRNA.

TESTI4023762//Trichohyalin.

TESTI4025920//B29 protein [Homo sapiens]

TESTI4039659//DnaJ homolog subfamily B member 8 (mDJ6).

TESTI4044186//leucine-rich, glioma inactivated 1 [Mus musculus]

THYMU2011736//latent transforming growth factor beta binding protein 3

THYMU2032825//Mus musculus mRNA for Drctnnb1a, complete cds.

THYMU2038369//Mus musculus GTRGEO22 (Gtrgeo22) mRNA, complete cds.

THYMU3001991//ART-4 protein [Homo sapiens]

THYMU3006172//membrane bound C2 domain containing protein [Rattus norvegicus]

TLIVE2003225//CUB and Sushi multiple domains 1 [Homo sapiens]

TLIVE2004320//Homo sapiens PC2-glutamine-rich-associated protein (PCQAP) mRNA, complete cds.

TOVAR2002247//Homo sapiens partial partial mRNA for NICE-4 protein, clone 3114f17.

TRACH2023299//growth factor receptor bound protein 2-associated protein 2 [Mus musculus]

TRACH3000926//cardiac morphogenesis [Mus musculus]

TRACH3001427//p47 [Homo sapiens]

TRACH3006412//Homo sapiens COP9 constitutive photomorphogenic homolog subunit 7B

TRACH3034731//Ras association (RalGDS/AF-6) domain family 2

TUTER2002729//D6MM5E protein [Mus musculus]

UTERU1000031//G. gallus mRNA for tom-1B protein.

UTERU2006115//ALPHA-ADAPTIN A (CLATHRIN ASSEMBLY PROTEIN COMPLEX 2 ALPHA-A LARGE CHAIN) (100 KDA COATED VESICLE PROTEIN A) (PLASMA MEMBRANE ADAPTOR HA2/AP2 ADAPTIN ALPHA A SUBUNIT).

UTERU2031268//NY-REN-25 antigen [Homo sapiens].

UTERU2035452//NG3 [Homo sapiens]

UTERU3001059//ABC1 protein homolog, mitochondrial precursor.

UTERU3005585//rhophilin-like protein [Homo sapiens]

UTERU3009871//feminization 1 homolog a (C. elegans)

The 14 clones shown below are clones which were unassignable to any of the above-mentioned categories, but have been predicted to have some function based on homology search using their full-length nucleotide sequences. Clone Name and Definition in the result of homology search, demarcated by a double slash mark (//), are shown below.

ADRGL2000042//Homo sapiens CTCL tumor antigen se20-4 mRNA, complete cds.

BRACE3009127//oxysterol binding protein 2; oxysterol binding protein-like 1 [Homo sapiens]

BRACE3021148//DC12 protein [Homo sapiens]

BRAMY2040159//Homo sapiens MRIP-1 mRNA, complete cds.

BRAWH3007441//CAT56 protein [Homo sapiens]

CTONG3001501//Mus musculus glucocorticoid-induced gene 1 mRNA, complete cds.

HCHON2000508//Homo sapiens prostate antigen PARIS-1 mRNA, complete cds.

OCBBF2020048//95 kDa retinoblastoma protein binding protein; KIAA0661 gene product

PERIC2007068//Mus musculus mRNA for 1A13 protein.

TESTI4010382//cytoplasmic-dynein heavy chain 2 [Rattus norvegicus]

TESTI4011072//tudor domain containing 1 [Mus musculus]

TESTI4046240//sirtuin 7

UTERU2019534//Golgi apparatus protein 1 [Homo sapiens]

UTERU2028734//Mus musculus slp2-a mRNA for synaptotagmin-like protein 2-a delta 2S-III, complete cds.

Further, a polypeptide will not always belong solely to a single category of the above-described functional categories, and therefore, a polypeptide may belong to any of the predicted functional categories. Further analyses may yield additional functions for clones classified into these functional categories.

Detailed descriptions concerning each domain or motif can be found in websites linked from the websites of Pfam, InterPro (http://www.ebi.ac.uk/interpro/), PROSITE (http://www.expasy.ch/cgi-bin/prosite-list.pl), or such. This information can be found based on domain/motif names, and accession numbers of hit data obtained through domain searches of Pfam (http://www.sanger.ac.uk/Software/Pfam/index.shtml) (see Example 5) for amino acid sequences deduced from the 2,495 full-length clones of the present invention whose full-length nucleotide sequences have been determined. PROSITE in particular enables comparison of unique functional categories. The functions of polypeptides encoded by the 914 clones with hit data in Pfam were predicted and classified into the 13 functional categories described below. As a result, 661 clones were estimated to encode proteins belonging to these categories.

Secretory and/or membrane protein (87 clones)

Glycoprotein-related protein (85 clones)

Signal transduction-related protein (154 clones)

Transcription-related protein (115 clones)

Enzyme and/or metabolism-related protein (265 clones)

Cell division- and/or cell proliferation-related protein (13 clones)

Cytoskeleton-related protein (40 clones)

Nuclear protein and/or RNA synthesis-related protein (31 clones)

Protein synthesis- and/or transport-related protein (46 clones)

Cellular defense-related protein (seven clones)

Development and/or differentiation-related protein (two clones)

DNA- and/or RNA-binding protein (179 clones)

ATP- and/or GTP-binding protein (36 clones)

The clones predicted to belong to the category of secretory protein and/or membrane protein are the following 64 clones.

ASTRO2014923, ASTRO3000301, BRACE2005457, BRACE2014306, BRACE3001391, BRACE3014005, BRALZ2016085, BRAMY2040592, BRAWH2014662, BRHIP2004814, BRHIP3024118, BRTHA3002427, BRTHA3017848, BRTHA3018656, CTONG2009423, CTONG2013178, D3OST2002648, FEBRA2007708, FEBRA2008311, HCHON2001084, HCHON2001712, HCHON2004531, HCHON2005921, HSYRA2009102, KIDNE1000064, KIDNE2000832, NT2RI3006376, OCBBF2031167, OCBBF2035110, OCBBF2038317, PEBLM2002594, PERIC1000147, PERIC2009086, PROST1000184, SPLEN2012624, SPLEN2031547, SPLEN2033098, SPLEN2036326, TESTI1000257, TESTI1000390, TESTI2000644, TESTI2002036, TESTI2002928, TESTI2006648, TESTI2024567, TESTI2034520, TESTI4000014, TESTI4000724, TESTI4007163, TESTI4009881, TESTI4028880, THYMU2009425, THYMU2011548, THYMU2033079, THYMU2041015, TLIVE2000023, TLIVE2003381, TLIVE2007132, TRACH2006387, TRACH2007059, TRACH3004786, UTERU3000645, UTERU3004616, UTERU3006308

The following 23 clones are also predicted to belong to the category of secretory protein and/or membrane protein.

BRACE2029396, BRACE3005107, BRACE3010076, BRAMY2019111, BRAMY3004800, BRHIP3000017, FCBBF1000509, HCHON2000508, HEART2009680, IMR322013396, NT2RI2009583, NT2RI3000174, NT2RP8000521, OCBBF2030116, TESTI2029252, TESTI4013894, TESTI4032112, TESTI4041086, THYMU2035710, TKIDN2012771, TRACH3000420, UTERU2004299, TESTI4038779

The clones predicted to belong to the category of glycoprotein-related protein are the following 77 clones.

ADIPS2000088, BRACE2043142, BRACE2046295, BRACE3014005, BRAMY2005052, BRAMY4000277, BRAWH2007658, BRCAN2006063, BRSTN2004863, BRTHA3017589, BRTHA3017848, COLON2000568, COLON2004478, CTONG2000042, CTONG2013178, CTONG2024206, CTONG2024749, CTONG2025496, CTONG3001370, CTONG3003737, D3OST2002182, FEBRA2007708, HCHON2001084, HCHON2002676, HCHON2004531, HEART2001680, HLUNG2014262, LYMPB2000083, NESOP2001433, NOVAR2001108, NT2RI3006171, NT2RI3006340, NT2RI3007978, NT2RP7014005, OCBBF2010140, OCBBF2037598, PLACE5000171, PLACE6012574, PLACE7006051, PUAEN2009174, SMINT2002743, SMINT2010076, SMINT2011888, SMINT2015787, SPLEN2001599, SPLEN2015267, SPLEN2021701, SPLEN2037722, STOMA2004294, SYNOV3000231, SYNOV3000302, SYNOV4007521, SYNOV4007671, TBAES2003550, TESOP2005485, TESTI2005610, TESTI4006326, TESTI4021294, THYMU2005303, THYMU2019210, THYMU2023711, THYMU2027695, TRACH2007059, TRACH2022425, TRACH2022553, TRACH2022649, TRACH3002168, TRACH3005479, TRACH3005549, TRACH3006470, TRACH3035526, TRACH3036609, TSTOM2000442, UTERU2026090, UTERU3004616, UTERU3004992, UTERU3006308

The following eight clones are also predicted to belong to the category of glycoprotein-related protein.

BRAWH2006395, BRHIP3000017, NT2RI3007443, OCBBF3002654, TESTI2039060, TESTI4013894, TESTI4031745, TLIVE2001684

The clones predicted to belong to the category of signal transduction-related protein are the following 116 clones.

BLADE2007958, BNGH42007788, BRACE1000258, BRACE2008594, BRACE2041009, BRACE3001391, BRACE3006872, BRACE3011421, BRACE3024073, BRACE3027326, BRALZ2014484, BRAMY2001473, BRAMY2036567, BRAMY2042760, BRAMY2047751, BRAMY3001794, BRAMY3002803, BRAMY3005091, BRAMY3008466, BRAMY4000095, BRAWH3001326, BRAWH3002821, BRAWH3005912, BRCAN2002856, BRCAN2009432, BRCAN2016619, BRCAN2024451, BRCAN2028355, BRHIP2000819, BRHIP2005932, BRHIP3008405, BRHIP3025161, BRSSN2000684, BRSSN2004719, BRSTN2008418, BRSTN2013741, BRTHA3009037, BRTHA3013884, COLON2001721, CTONG2006798, CTONG3000084, CTONG3000657, CTONG3002127, D3OST3000169, DFNES2001108, DFNES2011499, FCBBF3007540, HCASM2001301, HCHON2000028, HCHON2006250, HHDPC1000118, HLUNG2001996, HLUNG2002465, KIDNE2001847, MESAN2006563, NHNPC2001816, NT2NE2003252, NT2RI2005166, NT2RI3000622, NT2RI3006673, NT2RP7005118, NT2RP7005529, NT2RP7009147, NT2RP7013795, NT2RP8000483, NTONG2003852, OCBBF2004826, OCBBF2004883, OCBBF2007028, OCBBF2008770, OCBBF2022351, OCBBF2037340, OCBBF2037547, PEBLM2004666, PLACE7008431, PROST2016462, PROST2018511, PUAEN2002616, PUAEN2005930, PUAEN2006328, PUAEN2009852, SYNOV2021320, TESOP2000801, TESOP2001166, TESTI2006648, TESTI2026505, TESTI2050137, TESTI2052693, TESTI4000079, TESTI4010713, TESTI4010831, TESTI4011956, TESTI4016882, TESTI4019843, TESTI4028059, THYMU2032014, THYMU2037226, THYMU2038615, THYMU3001234, THYMU3006172, THYMU3008436, TLIVE2009541, TRACH2009310, TRACH2021398, TRACH2023299, TRACH2025535, TRACH3009455, TRACH3034731, TSTOM2000553, UTERU1000337, UTERU2005621, UTERU2025025, UTERU2036089, UTERU2038251, UTERU3003523, UTERU3007419

The following 38 clones are also predicted to belong to the category of signal transduction-related protein.

BLADE2000579, BRACE3001058, BRACE3003053, BRACE3009127, BRAMY2040159, BRAMY3004800, BRAWH3009017, BRCAN2014229, BRHIP2026877, BRTHA2013610, CTONG3004550, FEBRA2001990, FEBRA2008692, HCHON2000508, MESAN2001770, NT2RI2005772, NT2RI3007443, NTONG2008093, OCBBF2005433, OCBBF2024284, OCBBF2034637, OCBBF3002654, TESOP2000390, TESTI2025924, TESTI2049956, TESTI4000319, TESTI4005317, TESTI4021482, TESTI4025268, TESTI4031745, THYMU2004139, THYMU2031249, TRACH2024408, UTERU2008040, UTERU2028734, UTERU3000402, UTERU3000738, UTERU3015412

The clones predicted to belong to the category of transcription-related protein are the following 27 clones.

BRACE2006319, BRACE3013576, BRAMY203010.9, BRAWH3005912, BRHIP3025161, CORDB1000140, CTONG1000467, HEART2001756, IMR322000127, IMR322000917, KIDNE1000064, NOVAR2000136, NT2NE2006531, NT2RI3007158, NT2RP7000466, OCBBF2036743, OCBBF3009279, PLACE6019385, TESTI2026505, TESTI2044796, TESTI2050987, TESTI4017001, TESTI4019140, TESTI4034912, THYMU2035735, TRACH2025749, TRACH3004840

The following 88 clones are also predicted to belong to the category of transcription-related protein.

BRACE2003609, BRACE3001058, BRACE3001113, BRACE3003026, BRAMY2035070, BRAMY2035449, BRAMY2035718, BRAMY2039341, BRAMY2045471, BRAWH3007441, BRHIP2017553, BRSTN2013354, CERVX2002013, CTONG1000113, CTONG2003348, CTONG2020374, CTONG2020378, CTONG2020411, CTONG2024031, CTONG2028758, CTONG3001501, CTONG3004726, DFNES2011192, FCBBF3027854, FEBRA2014122, FEBRA2027609, HCASM2003018, HCASM2003099, HCHON2000508, HCHON2000743, HCHON2004858, HSYRA2005628, MESAN2014412, MESAN2015501, NT2RI2008952, NT2RI2018448, NT2RI3000174, NT2RI3001132, NT2RI3002557, NT2RI3007167, NT2RI3007443, OCBBF2008144, OCBBF2009583, OCBBF2011669, OCBBF2019684, OCBBF2020048, OCBBF2024284, OCBBF2032274, OCBBF3000167, OCBBF3003761, SPLEN2016135, SPLEN2016781, SPLEN2036702, SYNOV2021953, SYNOV4002744, TESOP2001796, TESOP2005199, TESOP2006398, TESTI2008901, TESTI2034251, TESTI2037830, TESTI4000183, TESTI4000214, TESTI4006473, TESTI4008058, TESTI4008302, TESTI4013365, TESTI4014801, TESTI4015442, TESTI4017714, TESTI4025494, TESTI4025547, TESTI4028938, TESTI4029348, TESTI4031745, TESTI4032090, THYMU2006001, THYMU2028739, THYMU2031139, THYMU3001428, TRACH2007483, TRACH3000134, TRACH3003832, TRACH3007866, UTERU3001053, UTERU3014791, UTERU3017176, TESTI4038779

The clones predicted to belong to the category of enzyme and/or metabolism-related protein are the following 176 clones.

3NB692002806, ASTRO1000009, BLADE2005036, BLADE2008539, BRACE2005457, BRACE2008594, BRACE2014475, BRACE2018762, BRACE2035381, BRACE2043142, BRACE2047011, BRACE3004058, BRACE3007625, BRACE3009708, BRACE3011421, BRACE3015262, BRACE3024073, BRACE3025630, BRACE3027478, BRAMY2047746, BRAMY2047751, BRAMY3002803, BRAMY3004919, BRAMY3005091, BRAMY4000095, BRAWH2010000, BRAWH2014414, BRAWH2014662, BRAWH2016702, BRAWH3002821, BRAWH3003727, BRCAN2021028, BRCAN2024451, BRCAN2028355, BRCOC2003213, BRHIP2004359, BRHIP2026288, BRHIP3008183, BRHIP3025161, BRHIP3027137, BRSSN2000684, BRSTN2000872, BRSTN2004863, BRSTN2004987, BRTHA2012980, BRTHA3002401, BRTHA3008778, BRTHA3009037, BRTHA3009090, BRTHA3015815, BRTHA3016917, BRTHA3017848, BRTHA3018656, COLON2001721, CTONG2004062, CTONG2006798, CTONG2013178, CTONG2028124, CTONG3002127, CTONG3005325, CTONG3005648, D3OST2002182, FCBBF3004502, FCBBF3013307, FEBRA2007708, FEBRA2008468, FEBRA2026984, HCASM2001301, HCASM2002918, HCHON2002676, HCHON2004007, HCHON2004531, HEART2006131, HHDPC1000118, HLUNG1000017, KIDNE2000832, KIDNE2006580, MESAN2012054, NOVAR2000136, NT2NE2003252, NT2NE2006909, NT2RI2004618, NT2RI3004510, NT2RI3006673, NT2RI3007978, NT2RI3008652, NT2RP7010599, NT2RP7014005, NT2RP7017474, NTONG2000413, OCBBF2004826, OCBBF2006058, OCBBF2019823, OCBBF2025527, OCBBF2031167, OCBBF2037340, OCBBF2037547, OCBBF2037638, PERIC2009086, PLACE7002641, PLACE7008431, PROST2017367, PUAEN2007044, PUAEN2009795, PUAEN2009852, SPLEN2010912, SPLEN2015679, SPLEN2030335, SYNOV4002392, SYNOV4002883, TBAES2003550, TESOP2000801, TESOP2004114, TESOP2009121, TESTI1000257, TESTI1000545, TESTI2002618, TESTI2006648, TESTI2040018, TESTI2049469, TESTI2053621, TESTI4000288, TESTI4000349, TESTI4001148, TESTI4001527, TESTI4001561, TESTI4002552, TESTI4006819, TESTI4007382, TESTI4007810, TESTI4008429, TESTI4010713, TESTI4010851, TESTI4012448, TESTI4012679, TESTI4013369, TESTI4016925, TESTI4018835, TESTI4020920, TESTI4021478, TESTI4022716, TESTI4026510, TESTI4028059, TESTI4029836, TESTI4032895, TESTI4034432, TESTI4036909, THYMU2006420, THYMU3000133, THYMU3001379, THYMU3004835, THYMU3006172, THYMU3008436, TLIVE2002336, TRACH2006387, TRACH2009310, TRACH2019473, TRACH2022425, TRACH2023299, TRACH3005479, TRACH3006470, TRACH3007479, TRACH3008093, TRACH3008629, TRACH3036193, TSTOM2000553, UTERU2005621, UTERU2017762, UTERU2025025, UTERU2033375, UTERU3000828, UTERU3001240, UTERU3001585, UTERU3003116, UTERU3005460, UTERU3005907

The following 89 clones are also predicted to belong to the category of enzyme and/or metabolism-related protein.

BLADE2000579, BRACE2039823, BRACE3003053, BRAMY2038516, BRAMY2040159, BRAWH1000369, BRCAN2003070, BRCAN2014229, BRCOC2019841, BRHIP2005724, BRHIP2008389, BRHIP2026877, BRHIP3000240, BRHIP3026052, BRTHA2002133, BRTHA2002702, BRTHA2007060, BRTHA2010033, BRTHA2013426, BRTHA2013610, BRTHA2017364, BRTHA2018011, BRTHA3000296, CTONG2004000, CTONG2016942, CTONG2020374, CTONG2024031, CTONG3002552, CTONG3003598, CTONG3004550, FCBBF1000509, FEBRA2008692, HCASM2002754, HCASM2003099, HCASM2003357, HLUNG2015418, HLUNG2015548, IMR322013731, MESAN2005303, NT2RI2005772, NT2RI2008952, NT2RI3000174, NT2RI3007443, NT2RP7008435, NTONG2008093, OCBBF2006987, OCBBF2034637, OCBBF3002654, PLACE7000333, PLACE7000502, PROST2000452, SPLEN2039311, STOMA2003158, SYNOV2013637, TESOP2000390, TESTI2015626, TESTI2025924, TESTI2026647, TESTI2035981, TESTI2036288, TESTI2039060, TESTI2049956, TESTI4000155, TESTI4001984, TESTI4006473, TESTI4010382, TESTI4011072, TESTI4014801, TESTI4017714, TESTI4021482, TESTI4025547, TESTI4025865, TESTI4026207, TESTI4028958, TESTI4029690, TESTI4031745, TESTI4032090, THYMU2004139, THYMU2031139, THYMU2031249, THYMU2040925, TKIDN2012771, TLIVE2002046, TLIVE2007607, TRACH3000420, TRACH3007866, UTERU2019534, UTERU2028734, UTERU3000738

The clones predicted to belong to the category of cell division and/or cell proliferation-related protein are the following ten clones.

BRAWH2001940, CTONG3001123, HCHON2001217, PROST2008993, TBAES2001171, TESTI4021294, TESTI4035498, UTERU1000024, UTERU3002993, UTERU3003523

The following three clones are also predicted to belong to the category of cell division and/or cell proliferation-related protein.

BRACE2029396, BRAWH2010552, TESTI4013365

The clones predicted to belong to the category of cytoskeleton-related protein are the following 36 clones.

BRACE2026836, BRACE2045300, BRAWH3000314, BRSTN2004863, BRTHA2004978, BRTHA3003449, BRTHA3005046, COLON2002520, CORDB2000541, CTONG3002674, FCBBF3012288, HCHON2001577, HLUNG2017350, HSYRA2005456, HSYRA2009075, NT2RI3006340, NT2RI3006673, NT2RI3007291, OCBBF2037598, PLACE5000282, TESTI2003347, TESTI2034767, TESTI4000288, TESTI4007778, TESTI4009160, TESTI4018886, TESTI4030603, TESTI4034632, TESTI4035063, THYMU1000496, THYMU2008725, TRACH2005811, TRACH2007059, UTERU2007724, UTERU2035745, UTERU3004616

The following four clones are also predicted to belong to the category of cytoskeleton-related protein.

NT2RI2005772, OCBBF2006987, SPLEN2030847, TESTI4026207

The clones predicted to belong to the category of nuclear protein and/or RNA synthesis-related protein are the following 20 clones.

BRACE3024073, BRAWH2001940, BRCOC2003213, BRSTN2004987, BRTHA3016917, CTONG3009028, FCBBF3013307, FEBRA2026984, SPLEN2010912, TBAES2001171, TESTI2040018, TESTI4019566, TESTI4022716, TESTI4026510, TESTI4036909, THYMU3000133, TRACH2023299, TRACH3036193, UTERU1000024, UTERU3002993

The following eleven clones are also predicted to belong to the category of nuclear protein and/or RNA synthesis-related protein.

BRACE3003053, BRCAN2002473, BRTHA2017364, NT2RI2008952, NT2RI3000174, TESTI2026647, TESTI2035981, TESTI4000155, TESTI4006473, TESTI4010382, TESTI4025547

The clones predicted to belong to the category of protein synthesis and/or transport-related protein are the following 29 clones.

BRACE2014306, BRACE3008720, BRAWH3000491, BRCAN2009432, BRHIP2000920, BRTHA3013884, CTONG2013178, HCHON2004531, HLUNG1000017, HLUNG2013851, HSYRA2005496, NT2NE2006909, NT2RI3006340, OCBBF2007068, OCBBF2031167, PUAEN2009795, TBAES2001229, TBAES2004055, TESTI2051867, TESTI4000014, TESTI4000349, TESTI4009608, TESTI4010851, TESTI4034632, TRACH3007479, TRACH3036193, UTERU2017762, UTERU2019940, UTERU2033375

The following 17 clones are also predicted to belong to the category of protein synthesis and/or transport-related protein.

BLADE2000579, BRACE3003053, BRCAN2003070, BRTHA2018011, BRTHA3000296, CTONG2016942, MESAN2005303, NT2RI3002557, NT2RP7008435, PERIC2007068, PLACE7000502, PROST2000452, TESTI4001984, TESTI4017714, THYMU2004284, TRACH3000420, TRACH3007866

The clones predicted to belong to the category of cellular defense-related protein are the following four clones.

BRTHA2015878, CTONG3000084, NT2RI3002842, PEBLM2004666

The following three clones are also predicted to belong to the category of cellular defense-related protein.

BRCAN2002473, NT2RI3007167, TRACH3002561

The clones predicted to belong to the category of development and/or differentiation-related protein are the following one clone.

TESTI4014924

The clones predicted to belong to the category of DNA-binding and/or RNA-binding protein are the following 67 clones.

BRACE2006319, BRACE2047011, BRACE3004150, BRACE3013576, BRACE3024073, BRAMY2030109, BRAWH3005912, BRCAN2002562, BRCOC2003213, BRHIP2021615, BRHIP3008183, BRHIP3025161, BRSTN2004987, BRTHA2018707, BRTHA3016917, CORDB1000140, CTONG1000467, CTONG3000084, CTONG3003972, CTONG3008831, CTONG3009028, FCBBF3013307, FEBRA2026984, HEART2001756, HLUNG2013851, IMR322000127, IMR322000917, KIDNE1000064, NT2NE2006531, NT2RI3003382, NT2RI3007158, NT2RP7000466, NT2RP7004123, OCBBF2036743, OCBBF3009279, PLACE6019385, SPLEN2006122, SPLEN2010912, TESOP2009121, TESTI1000390, TESTI2014716, TESTI2026505, TESTI2040018, TESTI2044796, TESTI2050987, TESTI4007810, TESTI4009374, TESTI4011745, TESTI4012679, TESTI4017001, TESTI4019140, TESTI4019566, TESTI4022716, TESTI4026510, TESTI4034432, TESTI4034912, TESTI4036909, THYMU2035319, THYMU2035735, THYMU3000133, TLIVE2002336, TRACH2023299, TRACH2025749, TRACH3004840, TRACH3036193, UTERU2026025, UTERU3009490

The following 112 clones are also predicted to belong to the category of DNA-binding and/or RNA-binding protein.

BLADE2006830, BRACE2003609, BRACE3001058, BRACE3001113, BRACE3003026, BRACE3003053, BRACE3010076, BRAMY2035070, BRAMY2035449, BRAMY2039341, BRAMY2045471, BRAWH1000369, BRAWH3007441, BRHIP2017553, BRSTN2013354, BRTHA2002133, BRTHA2002702, BRTHA2017364, BRTHA2017972, CERVX2002013, CTONG1000113, CTONG2003348, CTONG2015596, CTONG2020374, CTONG2020378, CTONG2020411, CTONG2024031, CTONG2028758, CTONG3001501, CTONG3004726, DFNES2011192, FCBBF1000509, FCBBF3027854, FEBRA2014122, FEBRA2027609, HCASM2003018, HCASM2003099, HCASM2009424, HCHON2000508, HCHON2000743, HCHON2004858, HSYRA2005628, IMR322013731, MESAN2014412, MESAN2015501, NT2RI2008952, NT2RI2018448, NT2RI2027157, NT2RI3000174, NT2RI3001132, NT2RI3002557, NT2RI3007167, NT2RI3007443, OCBBF2006987, OCBBF2008144, OCBBF2009583, OCBBF2011669, OCBBF2019684, OCBBF2020048, OCBBF2024284, OCBBF2032274, OCBBF2034637, OCBBF3000167, OCBBF3003761, PERIC2007068, SPLEN2016135, SPLEN2016781, SPLEN2036702, STOMA2003158, SYNOV2021953, SYNOV4002744, TESOP2001796, TESOP2005199, TESOP2006398, TESTI2008901, TESTI2026647, TESTI2034251, TESTI2035981, TESTI2037830, TESTI4000155, TESTI4000183, TESTI4000214, TESTI4006473, TESTI4008058, TESTI4008302, TESTI4010382, TESTI4013365, TESTI4014801, TESTI4015442, TESTI4017714, TESTI4025494, TESTI4025547, TESTI4026207, TESTI4028938, TESTI4028958, TESTI4029348, TESTI4031745, TESTI4032090, THYMU2006001, THYMU2028739, THYMU2031139, THYMU3001428, TKIDN2012771, TLIVE2007607, TRACH2007483, TRACH3000134, TRACH3003832, TRACH3007866, UTERU3001053, UTERU3014791, UTERU3017176, TESTI4038779

The clones predicted to belong to the category of ATP binding and/or GTP-binding protein are the following 28 clones.

BRACE3008720, BRACE3009708, BRAMY2047746, BRAMY3004919, BRAWH2014662, BRAWH2016702, BRCAN2009432, BRCAN2024451, BRSTN2013741, BRTHA3008778, BRTHA3009090, CTONG2004062, CTONG2028124, HCHON2004007, OCBBF2037340, SPLEN2030335, TESTI4000288, TESTI4001148, TESTI4002552, TESTI4008429, TESTI4018835, TESTI4021478, TESTI4029836, THYMU2036459, THYMU3001379, TRACH2001549, UTERU3000828, UTERU3001240

The following eight clones are also predicted to belong to the category of ATP binding and/or GTP-binding protein. BRCAN2014229, BRHIP2008389, CTONG3004550, FEBRA2001990, IMR322013396, IMR322013731, MESAN2001770, TESTI4000319

The following 208 clones have hit data in Pfam (see Example 5), and each has a functional domain or motif. It is currently unclear as to which of the above-described categories each of these clones belong. However, if data on polypeptides with a similar domain or motif can be accumulated, and their functions clarified in more detail, they may be classified into any of the above-described categories.

3NB692002685, 3NB692008729, ASTRO2003960, BNGH42003570, BRACE2010489, BRACE2015314, BRACE2016981, BRACE2027258, BRACE2030341, BRACE2035441, BRACE2038329, BRACE2042550, BRACE2044286, BRACE3000071, BRACE3000973, BRACE3001002, BRACE3003192, BRACE3604772, BRACE3004880, BRACE3008137, BRACE3008384, BRACE3009090, BRACE3010397, BRACE3015521, BRACE3016884, BRACE3019084, BRAMY2004771, BRAMY2019300, BRAMY2021498, BRAMY2031317, BRAMY2039872, BRAMY2046989, BRAMY3004224, BRAMY3005932, BRAWH1000127, BRAWH2001395, BRAWH2014954, BRAWH3000078, BRAWH3001891, BRAWH3002574, BRAWH3002600, BRAWH3008341, BRCAN2002948, BRCAN2009203, BRCAN2015464, BRCAN2017717, BRCOC2001505, BRCOC2016525, BRHIP2003786, BRHIP2005236, BRHIP2007616, BRHIP2009414, BRHIP3000339, BRHIP3008313, BRSTN2001067, BRTHA2000855, BRTHA2005579, BRTHA2007122, BRTHA2008527, BRTHA2009311, BRTHA2010884, BRTHA2013262, BRTHA2014792, BRTHA2015406, BRTHA2016496, BRTHA2018591, BRTHA2018624, BRTHA2019048, BRTHA3003074, BRTHA3008310, CTONG1000341, CTONG2001877, CTONG2008233, CTONG2017500, CTONG2020026, CTONG2028687, CTONG3000686, CTONG3004072, CTONG3006067, CTONG3006186, CTONG3009385, DFNES2000146, DFNES2005266, FCBBF3009888, FCBBF3012170, FEBRA2000253, FEBRA2007801, FEBRA2021571, FEBRA2024150, HCHON2004776, HEART1000139, HEART2006909, HEART2010495, HLUNG2000014, HLUNG2002958, HLUNG2011298, IMR322006495, KIDNE2000846, KIDNE2001361, KIDNE2011635, KIDNE2012945, NESOP2001656, NT2RI2008724, NT2RI2025909, NT2RI2025957, NT2RI3007543, NT2RP7000359, NT2RP7004027, NT2RP7011570, NT2RP8000296, NTONG2005277, NTONG2006354, NTONG2007517, OCBBF2006764, OCBBF2010416, OCBBF2020838, OCBBF2021323, OCBBF2033869, PERIC2001228, PERIC2003720, PLACE6020031, PLACE7000514, PROST2018090, RECTM2000433, SKMUS2006394, SMINT1000192, SPLEN2002147, SPLEN2002467, SPLEN2031780, SPLEN2034081, SPLEN2036821, SYNOV2005448, SYNOV2005817, SYNOV2006430, SYNOV2014400, SYNOV4007553, SYNOV4008440, TESOP2001953, TESTI2000443, TESTI2004700, TESTI2027019, TESTI4000462, TESTI4000970, TESTI4002491, TESTI4006546, TESTI4007064, TESTI4011484, TESTI4012406, TESTI4015471, TESTI4016110, TESTI4017137, TESTI4017575, TESTI4018152, TESTI4018555, TESTI4020092, TESTI4023555, TESTI4025920, TESTI4026192, TESTI4027557, TESTI4028429, TESTI4028612, TESTI4028983, TESTI4030505, TESTI4038492, TESTI4039659, TESTI4041053, TESTI4044084, TESTI4046487, TESTI4046819, THYMU2004693, THYMU2011736, THYMU2016204, THYMU2027734, THYMU2038369, THYMU2038797, THYMU3000028, THYMU3003212, THYMU3003763, THYMU3007137, THYMU3008171, TLIVE2002338, TLIVE2002690, TLIVE2003225, TLIVE2008229, TRACH2001443, TRACH3001427, TRACH3003379, TRACH3008713, TRACH3035235, TUTER2000425, UTERU1000031, UTERU2006115, UTERU2006568, UTERU2019706, UTERU2035328, UTERU2035331, UTERU2035452, UTERU3001652, UTERU3001766, UTERU3001988, UTERU3002667, UTERU3003178, UTERU3005585, UTERU3007640, UTERU3008660, UTERU3009871, UTERU3009979, UTERU3015500

Likewise, the following 45 clones also had hit data in Pfam (see Example 5), although it remains unclear as to which of the above-described categories each clone belongs. When data on polypeptides with a similar domain or motif are accumulated, and their functions are clarified in more detail, these clones may also be classified into any of the above-described categories.

3NB692004724//KRAB box//Integrase core domain

ADRGL2000042//Nucleosome assembly protein (NAP)

BRACE2037299//Integrase core domain

BRALZ2017844//Homeobox domain

BRAWH2006207//KRAB box

BRCAN2002854//SAP domain

BRHIP2006617//TPR Domain//TPR Domain

BRHIP2012360//XPG N-terminal domain//XPG I-region

BRHIP3008314//Sir2 family

BRTHA2016318//KE2 family protein

CTONG2019822//Hepatitis C virus core protein

FCBBF3010361//Fork head domain

FEBRA2006519//Thrombospondin type 1 domain//Thrombospondin type 1 domain

FEBRA2028256//EGF-like domain//EGF-like domain//EGF-like domain//EGF-like domain//EGF-like domain//TB domain//EGF-like domain//EGF-like domain//EGF-like domain//EGF-like domain//EB module//Squash family of serine protease inhibitors//EGF-like domain//EGF-like domain

FEBRA2028516//GRIP domain

HCASM2008536//XRCC1 N terminal domain

IMR322007078//UBA domain

IMR322008651//Helix-hairpin-helix motif.

LIVER2000247//Sodium

OCBBF2003327//Thrombospondin type 1 domain//Thrombospondin type 1 domain//Thrombospondin type 1 domain

PROST2009320//LIM domain containing proteins//LIM domain containing proteins

PUAEN2006335//Formin Homology 2 Domain

SKMUS2003194//SAP domain

SPLEN2039379//Transthyretin precursor (formerly prealbumin)

SYNOV1000256//Leucine Rich Repeat//BAH domain//Leucine Rich Repeat//Leucine Rich Repeat//Leucine Rich Repeat

SYNOV2006620//Nuclear transition protein 2

SYNOV4003981//Somatomedin B domain//WAP-type (Whey Acidic Protein) ‘four-disulfide core’//Hemopexin//Hemopexin

SYNOV4005889//Apolipoprotein A1/A4/E family

TESOP2006865//KRAB box

TESTI1000266//Integrase core domain

TESTI2050780//Kazal-type serine protease inhibitor domain

TESTI4000137//Domain of unknown function

TESTI4024387//GDP dissociation inhibitor

TESTI4029528//RanBP1 domain.

TESTI4038721//Squash family of serine protease inhibitors

TESTI4046240//Sir2 family

THYMU2035078//Domain of unknown function DUF27

THYMU3000269//FAD binding domain

THYMU3000360//Integrase core domain

TRACH1000212//TSC-22/dip/bun family

TRACH2000862//Guanylate-binding protein

TRACH2019672//CRAL/TRIO domain.

TRACH2024559//IQ calmodulin-binding motif//IQ calmodulin-binding motif

UTERU2032279//Serpins (serine protease inhibitors)

UTERU2033577//KRAB box

The function of a motif or domain may sometimes belong to more than one of the above-described functional categories, and there is also the possibility that such a motif or domain may be predicted to belong to every functional category. As new polypeptide data are accumulated and novel domains and motifs are found, a new functional domain or motif may be identified by re-analyzing deduced amino acid sequences in homology searches using updated databases. Thus in the future, the remaining clones, for which there are currently no hit data, may be classified into any of the above-described categories.

Since the polypeptides encoded by clones of the present invention contain full-length amino acid sequences, it is possible to analyze their biological activity and effect on cellular conditions such as cell proliferation and differentiation, by expressing the polypeptides as recombinant polypeptides using an appropriate expression system, injecting the recombinant into a cell, or raising a specific antibody against that polypeptide.

The biological activities of respective polypeptides can be analyzed by the methods as shown below.

Secretory Protein, Transmembrane Protein:

“Ion Channels” (Ed., R. H. Ashley, 1995) of “The Practical Approach Series” (IRL PRESS),
“Growth Factors” (Eds., I. McKay, I. Leigh, 1993),
“Extracellular Matrix” (Eds., M. A. Haralson, J. R. Hassell, 1995);
   Glycoprotein-Related Protein:
“Glycobiology” (Eds., M. Fukuda, A. Kobata, 1993) of “The Practical Approach Series” (IRL PRESS),
“Glycoprotein Analysis in Biomedicine” (Ed., Elizabeth F. Hounsell, 1993) of “Method in Molecular Biology” (Humana Press) series;
   Signal Transduction-Related Protein:
“Signal Transduction” (Ed., G. Milligan, 1992) of “The Practical Approach Series” (IRL PRESS),
“Protein Phosphorylation” (Ed., D. G. Hardie, 1993), or
“Signal Transduction Protocols” (Eds., David A. Kendall, Stephen J. Hill, 1995) of “Method in Molecular Biology” (Humana Press) series;
   Transcription-Related Protein:
“Gene Transcription” (Eds., B. D. Hames, S. J. Higgins, 1993) of “The Practical Approach Series” (IRL PRESS),
“Transcription Factors” (Ed., D. S. Latchman, 1993);
   Enzyme and/or Metabolism-Related Protein:
“Enzyme Assays” (Eds., ROBERT EISENTHAL and MICHAEL J. DANSON, 1992) of “The Practical Approach Series” (IRL PRESS);
   Cell Division and/or Cell Proliferation-Related Protein:
“Cell Growth, Differentiation and Senescence” (Ed., GEORGE STUDZINSKI, 2000) of “The Practical Approach Series” (IRL PRESS);
   Cytoskeleton-Related Protein:
“Cytoskeleton: Signalling and Cell Regulation” (Eds., KERMIT L. CARRAWAY and CAROLIE A. CAROTHERS CARRAWAY, 2000) of “The Practical Approach Series” (IRL PRESS),
“Cytoskeleton Methods and Protocols” (Ed., Gavin, Ray H., 2000) of “Method in Molecular Biology” (Humana Press) series;
   Nuclear Protein and/or RNA Synthesis-Related Protein:
“Nuclear Receptors” (Ed., DIDIER PICARD, 1999) of “The Practical Approach Series” (IRL PRESS),
“RNA Processing” (Eds., STEPHEN J. HIGGINS and B. DAVID HAMES, 1994);
   Protein Synthesis and/or Transport-Related Protein:
“Membrane Transport” (Ed., STEPHEN A. BALDWIN, 2000) of “The Practical Approach Series” (IRL PRESS),
“Protein Synthesis Methods and Protocols” (Eds., Martin, Robin, 1998) of “Method in Molecular Biology” (Humana Press) series;
   Cellular Defense-Related Protein:
“DNA Repair Protocols” (Henderson, Daryl S., 1999) of “Method in Molecular Biology” (Humana Press) series,
“Chaperonin Protocols” (Eds., Schneider, Christine, 2000);
   Development and/or Differentiation-Related Protein:
“Developmental Biology Protocols” (Eds., ROBERT EISENTHAL and MICHAEL J. DANSON, 1992) of “Method in Molecular Biology” (Humana Press) series;
   DNA- and/or RNA-Binding Protein:
“DNA-Protein Interactions Principles and Protocols” (Eds., Kneale, G. Geoff, 1994) of “Method in Molecular Biology” (Humana Press) series,
“RNA-Protein Interaction Protocols” (Eds., Haynes, Susan R., 1999);
   ATP- and/or GTP-Binding Protein:
“Signal Transduction Protocols” (Eds., David A. Kendall, Stephen J. Hill, 1995) of “Method in Molecular Biology” (Humana Press) series.

When other techniques are used, the activity of a polypeptide can be analyzed according to the description in Methods in Enzymology (Academic Press).

In the above-described categorization, a clone predicted to belong to the secretory and/or membrane protein category refers to a clone having hit data in a homology search with some annotation to suggest that the clone encodes a secretory and/or membrane protein, such as a growth factor, cytokine, hormone, signal, transmembrane, membrane, extracellular matrix, receptor, G-protein coupled receptor, ionic channel, voltage-gated channel, calcium channel, cell adhesion, collagen, and connective tissue protein; or a clone in which the results of PSORT and SOSUI analyses for deduced ORF suggest the presence of a nucleotide sequence encoding a signal sequence or transmembrane region; or a clone in which the results of a domain/motif search with Pfam suggest the presence of domains/motifs that suggest receptors, ion channels, hormones, or growth factors, for example, seven-transmembrane receptors, pancreatic hormone peptides, ion transport proteins, or fibroblast growth factors.

A clone predicted to belong to the glycoprotein-related protein category means a clone having hit data in a homology search with some annotation, such as glycoprotein, suggesting that the clone encodes a glycoprotein-related protein; or a clone in which the results of a domain/motif search with Pfam indicate the presence of domains and motifs such as a glycoprotein or glycosyltransferase that suggest the involvement of glycobiology, for example, immunoglobulin domain or glycosyl transferases group 1.

A clone predicted to belong to the signal transduction-related protein category means a clone having hit data in a homology search with some annotation, such as serine/threonine-protein kinase, tyrosine-protein kinase, SH3 domain, and SH2 domain, suggesting that the clone encodes a signal transduction-related protein; or a clone in which the results of a domain/motif search with Pfam suggest the presence of domains and motifs that suggest a protein kinase, dephosphoenzyme, SH2 domain, or small G protein, for example, eukaryotic protein kinase domain, protein phosphatase 2C, or Ras family.

A clone predicted to belong to the transcription-related protein category means a clone having hit data in a homology search with some annotation, such as transcription regulation, zinc finger, and homeobox, suggesting that the clone encodes a transcription-related protein; or a clone in which the results of a domain/motif search with Pfam suggest the presence of domains and motifs that suggest a transcription factor or transcription-controlling protein, for example, bZIP transcription factor, Zinc finger, or C2H2 type.

A clone predicted to belong to the category of disease-related protein means a clone having hit data in a homology search with some annotation, such as disease mutation and syndrome, suggesting that the clone encodes a disease-related protein; or a clone whose full-length nucleotide sequence has hit data in Swiss-Prot, GenBank, UniGene, nr or RefSeq, where that hit data corresponds to genes or polypeptides which have been deposited in the Online Mendelian Inheritance in Man (OMIM) (http://www.ncbi.nlm.nih.gov/Omim/), the human gene and disease database described later; or a clone in which the results of a domain/motif search with Pfam suggest the presence of domains and motifs, that suggest proteins with disease-specific expression or proteins involved in increasing or decreasing expression (depending on the disease), for example, Wilm's tumor protein or von Hippel-Lindau disease tumor suppressor protein.

A clone predicted to belong to the category of enzyme and/or metabolism-related protein means a clone having hit data in a homology search with some annotation, such as metabolism, oxidoreductase, and E. C. No. (Enzyme commission number), suggesting that the clone encodes an enzyme and/or metabolism-related protein; or a clone in which the results of a domain/motif search with Pfam suggests the presence of domains and motifs that suggest transferase, synthase, or hydrolase, for example, aldehyde dehydrogenase family, chitin synthase, or glucose-6-phosphate dehydrogenase.

A clone predicted to belong to the category of cell division and/or cell proliferation-related protein means a clone having hit data in a homology search with some annotation, such as cell division, cell cycle, mitosis, chromosomal protein, cell growth, and apoptosis, suggesting that the clone encodes a cell division and/or cell proliferation-related protein; or a clone in which the results of a domain/motif search with Pfam suggest the presence of domains and motifs that suggest cyclin or cell proliferation-controlling protein, for example, cyclin or cell division protein.

A clone predicted to belong to the category of cytoskeleton-related protein means a clone having hit data in a homology search with some annotation, such as structural protein, cytoskeleton, actin-binding, and microtubles, suggesting that the clone encodes a cytoskeleton-related protein; or a clone in which the results of a domain/motif search with Pfam suggest the presence of domains and motifs that suggest actin, kinesin, or fibronectin, for example, actin, fibronectin type I domain, or kinesin motor domain.

A clone predicted to belong to the category of nuclear protein and/or RNA synthesis-related protein means a clone having hit data in a homology search with some annotation, such as nuclear protein, RNA splicing, RNA processing, RNA helicase, and polyadenylation, suggesting that the clone encodes a nuclear protein and/or RNA synthesis-related protein; or a clone in which the results of a domain/motif search with Pfam suggest the presence of domains and motifs that suggest splicing factor, RNA synthase, or helicase, for example, hepatitis C virus RNA dependent RNA polymerase or DEAD/DEAH box helicase.

A clone predicted to belong to the category of protein synthesis and/or transport-related protein means a clone having hit data in a homology search with some annotation, such as translation regulation, protein biosynthesis, amino-acid biosynthesis, ribosomal protein, protein transport, and signal recognition particle, suggesting that the clone encodes a protein synthesis and/or transport-related protein; or a clone in which the results of a domain/motif search with Pfam suggest the presence of domains and motifs that suggest translation-relating protein, ubiquitin-relating protein, or ribosomal protein, for example, translation initiation factor SUI1, ubiquitin family, or ribosomal protein L16.

A clone predicted to belong to the category of cellular defense-related protein means a clone having hit data in a homology search with some annotation, such as heat shock, DNA repair, and DNA damage, suggesting that the clone encodes a cellular defense-related protein; or a clone in which the results of a domain/motif search with Pfam suggest the presence of domains and motifs that suggest chaperonin or DNA repair protein, for example, HSP90 protein or DNA mismatch repair protein.

A clone predicted to belong to the category of development and/or differentiation-related proteins means a clone having hit data in a homology search with some annotation, such as developmental protein, suggesting that the clone encodes a development and/or differentiation-related protein; or a clone in which the results of a domain/motif search with Pfam suggest the presence of domains and motifs that suggest organogenesis-relating protein, for example, floricaula/leafy protein.

A clone predicted to belong to the category of DNA- and/or RNA-binding protein means a clone having hit data in a homology search with some annotation, such as DNA-binding, RNA-binding, and such; or a clone in which the results of a domain/motif search with Pfam suggest the presence of domains and motifs that suggest DNA/RNA-relating enzyme group including transcription factor and DNA ligase or Zinc-finger relating protein, for example, transcription factor WhiB, B-box zinc finger, or tRNA synthetases class I (C).

A clone predicted to belong to the category of ATP- and/or GTP-binding protein means a clone having hit data in a homology search with some annotation, such as ATP-binding, GTP-binding, and such; or a clone in which the results of a domain/motif search with Pfam suggest the presence of domains and motifs that suggest ATP/GTP-relating enzyme group including ATPase or G protein, for example, E1-E2 ATPase or Ras family.

It is possible to perform functional analysis of a protein involved in a disease as described above. It is also possible to analyze correlation between a protein's expression or activity and a certain disease by using a specific antibody obtained by using the expressed protein. Alternatively, it is possible to utilize the OMIM database, which is a database of human genes and diseases, to analyze the protein. New information is constantly being deposited into this database. Therefore, it is possible that one skilled in the art will find a new relationship between a particular disease and a gene of the present invention by using the most up-to-date database. Proteins involved in diseases are useful for developing diagnostic markers or medicines for regulation of their expression and activity, or as gene therapy targets.

The proteins may have a variety of functions, including but not limited to the above 14 categories, such as secretory proteins, membrane proteins, signal transduction-related proteins, glycoprotein-related proteins, or transcription-related proteins. When searching OMIM using these keywords, the proteins are revealed to be involved in a great number of diseases (the results of the OMIM search for secretory and membrane proteins are shown below as an Example). Associations between proteins related to signal transduction or transcription and diseases are reported in “Transcription Factor Research-19991 (Fujii, Tamura, Morohashi, Kageyama, and Satake edit, (1999) Jikken-Igaku Zoukan, Vol. 17, No. 3), and “Gene Medicine” (1999) Vol. 3, No. 2). As another example and as described in “Biology of Cancer”, many proteins are involved in cancers, including enzymes and/or metabolism-related proteins, cytoskeleton-related proteins, cell division and/or cell proliferation-related proteins as well as secretory proteins, membrane proteins, signal transduction-related proteins, glycoprotein-related proteins, transcription-related proteins, (S. Matsubara, 1992) of Life Science series (Shokabo). As the above example clearly demonstrates, not only disease-related proteins but also secretory proteins, membrane proteins, signal transduction-related proteins, glycoprotein-related proteins, and transcription-related proteins are often involved in diseases, and thus such proteins can be useful targets in the field of medical industry.

The results of the OMIM search for secretory and membrane proteins are shown below. The keywords used were:

(1) secretion protein,

(2) membrane protein,

(3) channel, and

(4) extracellular matrix.

Only the OMIM accession numbers are shown in the search results. The first 50 accession numbers displayed in the search results are provided. Using this number, data showing the relationship between a disease and a gene or protein can be seen. OMIM data is renewed daily.

1) Secretion Protein

When searching under these keywords, 436 genes were registered as being associated with disease. The OMIM numbers for 50 of these genes are as follows.

*604667, *104760, *176860, *139320, *118910, *151675, *107400, *604029, #200100, *177061, *600946, *601693, *139250, *176880, *600998, *603850, *605083, *147572, *179513, *606055, *604028, *125950, *157147, *246700, *602926, *600560, *602421, *603215, 185860, *600174, *179512, *109270, *179511, *179510, *179509, *601146, *604710, *177020, *138120, *170280, *600626, *164160, *168470, *154545, *603831, *601652, *104311, *601489, *603062, *102720

2) Membrane Protein

When searching under these keywords, 1873 genes were registered as being associated with disease. The OMIM numbers for 50 of these genes are as follows.

*130500, *605704, *305360, *153330, *109270, *173610, *170995, *120920, *170993, *309060, *104776, *602333, *605703, *602690, *605943, *159430, *600897, *606867, *133090, *601178, *602413, *602003, *604405, *605940, *603237, *109280, *606958, *600378, *606959, *602173, *107776, *602334, *125305, *602335, *309845, *601134, *605731, *606795, *185881, *607178, *603177, *154045, *603214, *603718, *606909, *600594, *603241, *606629, *603657, *600182

3) Channel (Member of Membrane Proteins)

When searching under these keywords, 449 genes were registered as being associated with disease. The OMIM numbers for 50 of these genes are as follows.

*176266, *600724, *605427, *182390, *123825, *114208, *114206, *114205, *176267, *600053, *601784, *603749, *182392, *600937, *603415, *114204, *114209, *114207, *607370, *604528, *604527, *601011, *600760, *192500, *118425, *600228, *600359, *176261, *602235, *600761, *182389, *300008, *600877, *605692, *300338, *602232, *603537, *182391, *176263, *602343, *601328, *605874, *604385, *603939, *602208, *601534, *601958, *603220, *600504, *607368

4) Extracellular Matrix

When searching under these keywords, 267 genes were registered as being associated with disease. The OMIM numbers for 50 of these genes are as follows.

*605912, *602201, *603479, *604633, *601418, *601548, *115437, *154870, *120361, *602285, *600754, *602262, *134797, *602261, *603320, *603321, *604871, *604629, *601807, #154700, *128239, *600310, *605470, *185250, *178990, *603767, *120360, *185261, *116935, *607056, *253700, *190180, *600985, *188826, *193300, *276901, *308700, *120150, *602109, *120324, *600514, #177170, #247100, #116920, #200610, *605127, *601313, *601652, *120180, *154790

In addition to these, various keywords shown in the above-mentioned categorizations or others can be used in an OMIM search to reveal involvement in disease.

Further, the use of nucleotide sequences of cDNAs of the present invention enables the expression frequency of genes corresponding to those cDNAs to be analyzed. Gene function can be predicted based on information obtained by expression frequency analysis.

There are several methods for analyzing the expression level of genes involved in disease. Differences in gene expression levels between diseased and normal tissues can be studied by analytical methods using, for example, Northern blotting, RT-PCR, DNA microarrays, etc. (Experimental Medicine, Vol. 17, No. 8, 980-1056 (1999); Cell Engineering (additional volume) DNA Microarray and Advanced PCR Methods, Muramatsu & Nawa (eds.), Shujunsya (2000)). In addition to these analysis methods, computer analysis can be used to compare the nucleotide sequences of expressed genes, and hence to analyze expression frequency. For example, in the “BODYMAP” database, gene clones are randomly extracted from cDNA libraries of various tissues and/or cells, clones homologous to each other are assigned to a single cluster based on 3′-end nucleotide sequence homology information, genes are then classified into clusters, and the number of clones in each cluster is compared to gain information on expression frequency (http://bodymap.ims.u-tokyo.ac.jp/).

When these analytical methods result in observation of an explicit difference between gene expression levels in diseased tissues and normal tissues, it can be concluded that the gene is closely involved in the disease or disorder. When gene expression is explicitly different between normal cells and cells reproducing specific disease-associated features, even if they are not diseased tissues, it can be concluded that the gene is closely involved in a disease or disorder.

Of the 2,495 clones whose full-length nucleotide sequences were revealed, genes involved in a particular pathology or function were selected using the database shown below (see Example 7; “Expression frequency analysis in silico”). The database used in the analyses of the present invention contains the nucleotide sequences of 1,402,069 clones, a sufficiently large population for analysis. Sequence information in the database was obtained by randomly selecting cDNA clones from cDNA libraries derived from the various tissues and cells shown in Example 1, and determining the 5′-end sequences thereof.

The nucleotide sequence of each clone in this database was then categorized (clustered) based on nucleotide sequence homology determined with a search program. The number of clones belonging to each cluster of each library was determined and normalized; and the ratio of a certain gene in a cDNA library was determined. This analysis provided information on the expression frequency of a gene in the tissue or cell that was the source of the cDNA library.

In order to analyze the expression of genes corresponding to the nucleotide sequences of cDNAs of the present invention in tissues and cells, the libraries from the tissues or cells, which had been used in large-scale cDNA analyses, were taken as subjects for comparison of expression levels between different tissues or cells. Namely, expression frequency was analyzed by comparing the previously normalized values between tissues or cells from which were derived the 600 or more cDNA clones whose nucleotide sequences had been analyzed. This analysis showed that the cDNA clones corresponded to the genes involved in the pathologies and functions indicated below. Each value in Tables 2 to 24 indicated below represents a relative expression frequency; a higher value indicates a higher expression level. Genes included in these Tables do not indicate such a big difference between compared libraries, but when compared with other tissue- or gene-derived libraries based on Example 9, they indicate a significant difference. Thus, these genes are specific to a tissue or cell, and can be considered useful diagnostic markers for disease, as well as useful for analyzing molecular mechanisms.

Osteoporosis-Related Genes

Osteoporosis is a pathology in which bones are easily broken owing to an overall decrease in bone components. The onset involves the balance between the functions of osteoblast producing bone and osteoclast absorbing bone, namely bone metabolism. Genes involved in the increase of osteoclasts differentiating from precursor cells of monocyte/macrophage line (Molecular Medicine 38. 642-648. (2001)) are thus genes involved in osteoporosis relevant to bone metabolism.

Nucleotide sequence-based analysis was carried out to identify genes whose expression frequencies were higher or lower in CD34+ cells (cells expressing glycoprotein CD34) treated with osteoclast differentiation factor (Molecular Medicine 38. 642-648. (2001)), compared to untreated CD34+ cells, which are precursor cells in monocyte/macrophage lines. The result of comparative analysis of frequency between the cDNA libraries prepared from the RNA of CD34+ cells (CD34C), and from the RNA of CD34+ cells treated with the osteoclast differentiation factor (D30ST, D60ST or D90ST) (Table 2), showed that the genes whose expression levels differed between the two were 15 and two clones indicated below.

BRACE3013780, BRAMY2047420, BRSTN2016470, CTONG3008894, D3OST2002182, D3OST2002648, D3OST3000169, PEBLM2005183, PUAEN2009655, TESTI4000014, TESTI4010851, TRACH2023299, TRACH2025535, TRACH3001427, UTERU20.06137 HCHON2000508, TESTI2015626

These clones are involved in osteoporosis.

Genes Involved in Neural Cell Differentiation

Genes involved in neural cell differentiation are useful for treating neurological disease. Genes with varying expression levels in response to induction of cellular differentiation in neural cells are thought to be involved in neurological disease.

A survey was performed for genes whose expression levels varied in response to induction of differentiation (stimulation by retinoic acid (RA) or growth inhibitor treatment after RA stimulation) in cultured cells of a neural strain, NT2. The result of comparative analysis of cDNA libraries derived from undifferentiated NT2 cells (NT2RM) and cells subjected to differentiation treatment (NT2RP, NT2RI or NT2NE) (Table 3) showed that the genes whose expression levels differed between the two were 174 and 30 clones indicated below.

BNGH42007788, BRACE1000186, BRACE2006319, BRACE2014306, BRACE2015058, BRACE2044286, BRACE3010428, BRAMY2044078, BRAWH2014645, BRAWH2014662, BRAWH3002574, BRAWH3003992, BRAWH3005981, BRAWH3007592, BRCAN2009432, BRCAN2016619, BRCAN2028355, BRHIP2001074, BRHIP2007741, BRHIP2014228, BRHIP2024146, BRHIP3007586, BRHIP3018797, BRTHA2003461, BRTHA3000633, BRTHA3003490, COLON2001721, CTONG1000087, CTONG2008233, CTONG2020638, CTONG2028124, CTONG3003905, CTONG3008894, CTONG3009028, CTONG3009239, DFNES2011499, FCBBF3001977, FEBRA1000030, FEBRA2006396, FEBRA2007801, HCHON2000028, HCHON2000244, HCHON2001084, HCHON2001217, HCHON2001548, HCHON2006250, HEART1000074, HHDPC1000118, HSYRA2009075, IMR322000127, IMR322001380, KIDNE2000665, KIDNE2002252, MESAN2006563, MESAN2012054, MESAN2015515, NT2NE2003252, NT2NE2005890, NT2NE2006531, NT2NE2006909, NT2NE2008060, NT2RI2003993, NT2RI2004618, NT2RI2005166, NT2RI2006686, NT2RI2008724, NT2RI2009855, NT2RI2011422, NT2RI2011683, NT2RI2012659, NT2RI2012990, NT2RI2013357, NT2RI2014247, NT2RI2014551, NT2RI2014733, NT2RI2016128, NT2RI2018311, NT2RI2018883, NT2RI2019751, NT2RI2023303, NT2RI2025909, NT2RI2025957, NT2RI2027081, NT2RI2027396, NT2RI3000622, NT2RI3001263, NT2RI3001515, NT2RI3002303, NT2RI3002842, NT2RI3002892, NT2RI3003031, NT2RI3003095, NT2RI3003162, NT2RI3003382, NT2RI3003409, NT2RI3004381, NT2RI3004510, NT2RI3005202, NT2RI3005403, NT2RI3005724, NT2RI3006132, NT2RI3006171, NT2RI3006284, NT2RI3006340, NT2RI3006376, NT2RI3006673, NT2RI3006796, NT2RI3007065, NT2RI3007158, NT2RI3007291, NT2RI3007543, NT2RI3007757, NT2RI3007978, NT2RI3008055, NT2RI3008162, NT2RI3008652, NT2RI3008697, NT2RI3008974, NT2RI3009158, NT2RP7000359, NT2RP7000466, NT2RP7004027, NT2RP7004123, NT2RP7005118, NT2RP7005529, NT2RP7005846, NT2RP7009030, NT2RP7009147, NT2RP7009867, NT2RP7010128, NT2RP7010599, NT2RP7011570, NT2RP7013795, NT2RP7014005, NT2RP7015512, NT2RP7017365, NT2RP7017474, NT2RP7017546, NT2RP8000137, NT2RP8000296, NT2RP8000483, NTONG2005969, OCBBF2007028, OCBBF2037068, PLACE7000514, PUAEN2007044, SPLEN2002467, SPLEN2006122, SPLEN2028914, SPLEN2031547, SYNOV4002346, SYNOV4007671, SYNOV4008440, TESOP2002273, TESTI2003573, TESTI4000014, TESTI4009286, TESTI4010851, TESTI4012702, TESTI4029671, TESTI4037156, THYMU3000133, TRACH1000205, TRACH2005811, TRACH2007834, TRACH2025535, TRACH3001427, TRACH3002192, TRACH3004721, TRACH3008093, TRACH3008535, TRACH3008713, UTERU2002410, UTERU2023175

ADRGL2000042, BRACE2003609, BRACE3003026, BRHIP3000017, CTONG2020411, FCBBF1000509, FCBBF3027854, FEBRA2028516, HCHON2000508, IMR322001879, NT2RI2005772, NT2RI2008952, NT2RI2009583, NT2RI2018448, NT2RI2027157, NT2RI3000174, NT2RI3001132, NT2RI3002557, NT2RI3005928, NT2RI3007167, NT2RI3007443, NT2RP7008435, NT2RP8000521, OCBBF2006987, PERIC2007068, TESTI2015626, TESTI4015442, TLIVE2002046, TRACH3000134, TUTER2000057

These genes are neurological disease-related genes.

Genes Involved in Alzheimer's Disease

Alzheimer's disease is a cranial neurological disease characterized by memory loss. As the disease advances, patients can no longer support themselves and require nursing. Alzheimer's disease eventually leads to brain atrophication. Environmental factors such as stress, and vascular factors such as hypertension and cholesterolemia, are assumed but not confirmed to contribute to the onset of Alzheimer's disease. Genes whose expression levels differ between normal brain tissues and tissues affected with Alzheimer's disease are expected to be involved in Alzheimer's disease. Such genes can be used to elucidate the disease's onset mechanism and in genetic diagnosis. cDNA libraries derived from the cerebral cortex of Alzheimer patients (BRALZ and BRASW) and a library derived from the whole tissues of a normal brain (BRAWH) were analyzed and compared (Table 4). Genes whose expression levels differed between the two were the 250 clones and 41 clones listed below.

ASTRO1000009, BLADE2008398, BRACE1000186, BRACE1000258, BRACE1000533, BRACE2005457, BRACE2010489, BRACE2014657, BRACE2035381, BRACE2044286, BRACE2045954, BRACE3000787, BRACE3003192, BRACE3005499, BRACE3007480, BRACE3009237, BRACE3009724, BRACE3009747, BRACE3010428, BRACE3011271, BRACE3011421, BRACE3012364, BRACE3022769, BRACE3026735, BRACE3031838, BRALZ2011796, BRALZ2012183, BRALZ2012848, BRALZ2014484, BRALZ2016085, BRALZ2016498, BRALZ2017359, BRAMY2003008, BRAMY2005052, BRAMY2019300, BRAMY2019963, BRAMY2036567, BRAMY2037823, BRAMY2040592, BRAMY3002803, BRAMY3004224, BRAMY3005091, BRASW1000053, BRASW1000125, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCOC2003213, BRCOC2014033, BRCOC2020142, BRHIP2000920, BRHIP2005719, BRHIP2007741, BRHIP2014228, BRHIP2024146, BRHIP2026288, BRHIP3000339, BRHIP3006683, BRHIP3007586, BRHIP3008405, BRHIP3018797, BRSSN2000684, BRSSN2011738, BRSSN2014299, BRSTN2008052, BRSTN2015015, BRSTN2016470, BRTHA1000311, BRTHA2008335, BRTHA3002427, BRTHA3003490, BRTHA3008520, BRTHA3017848, COLON2001721, CTONG2017500, CTONG2028124, CTONG3000657, CTONG3001123, CTONG3009328, FCBBF2001183, FCBBF3001977, FEBRA2007544, FEBRA2007801, FEBRA2020886, FEBRA2028618, HCASM2007047, HCHON2000244, HCHON2000626, HCHON2001217, HCHON2002676, HCHON2006250, HEART1000074, HHDPC1000118, HLUNG2002465, IMR322000127, IMR322001380, IMR322002035, KIDNE2006580, MESAN2006563, MESAN2012054, MESTC1000042, NOVAR2001783, NT2NE2006909, NT2RI2008724, NT2RI2012659, NT2RI2014733, NT2RI3002892, NT2RI3006284, NT2RI3006673, NT2RI3007543, NT2RI3008055, NT2RP7005529, NT2RP7009147, NT2RP7014005, NT2RP7017474, NTONG2005969, OCBBF200179.4, OCBBF2006005, OCBBF2006764, OCBBF2007028, OCBBF2007114, OCBBF2010140, OCBBF2021286, OCBBF2023162, OCBBF2024850, OCBBF2028935, OCBBF2036743, OCBBF2038317, OCBBF3000483, OCBBF3008230, PEBLM2004666, PLACE6001185, PUAEN2005930, PUAEN2006701, PUAEN2007044, PUAEN2009655, SMINT2001818, SPLEN2028914, SPLEN2031424, SPLEN2031547, SPLEN2034781, SPLEN2036932, SYNOV2014400, SYNOV4002346, SYNOV4002883, SYNOV4007430, SYNOV4007671, SYNOV4008440, TESOP2002273, TESOP2002451, TESTI4000014, TESTI4000209, TESTI4001100, TESTI4006137, TESTI4008797, TESTI4009286, TESTI4010851, TESTI4013817, TESTI4014694, TESTI4021478, TESTI4022936, TESTI4024420, TESTI4027821, THYMU2001090, THYMU2033308, THYMU2035735, THYMU2039315, THYMU3001234, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3001427, TRACH3002192, TRACH3004068, TRACH3004721, TRACH3005294, TRACH3007479, TRACH3008093, TRACH3009455, UTERU2005621, UTERU2006115, UTERU2019706, UTERU2023039, UTERU2026203, UTERU3005230, UTERU3007640, UTERU3009871

ADRGL2000042, BLADE2006830, BRACE2003609, BRALZ2017844, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRHIP3026052, BRTHA2018443, BRTHA3003000, CTONG2020374, CTONG2020378, CTONG2024031, FCBBF1000509, FEBRA2001990, FEBRA2006519, FEBRA2028516, HCHON2000743, IMR322001879, NT2RI2009583, OCBBF2008144, PERIC2007068, PUAEN2006335, SPLEN2039379, TESTI4001984, TESTI4008058, TESTI4025268, TESTI4032090, THYMU3000360, TLIVE2002046, TRACH3000134, UTERU2021820, UTERU2028734

These genes are involved in Alzheimer's disease.

Genes Involved in Parkinson's Disease

Parkinson's disease is a cranial neurological disease characterized by impaired production of the neurotransmitter dopamine in the substantia nigra in the brain. This results in dyskinesia, such as hand tremors, and impaired body movement due to muscular rigidity. Normally, the number of brain neurons gradually decreases with age. However, compared to healthy people, patients with Parkinson's disease experience a rapid and marked decrease in the number of neurons in their substantia nigra. Genes whose expression levels differ between tissues of the whole brain and the nigra are expected to be involved in Parkinson's disease. These genes exhibit nigra-specific alterations in their expression levels, and can be used to elucidate the disease onset mechanism and in gene diagnosis. cDNA libraries derived from the substantia nigra (BRSSN) and a library derived from whole tissues of a normal brain (BRAWH) were analyzed and compared (Table 5). Genes whose expression levels differed between the two were the 250 clones and 40 clones listed below.

ASTRO1000009, BLADE2008398, BRACE1000186, BRACE1000258, BRACE1000533, BRACE2005457, BRACE2010489, BRACE2014657, BRACE2035381, BRACE2044286, BRACE2045954, BRACE3000787, BRACE3003192, BRACE3005499, BRACE3007480, BRACE3009237, BRACE3009724, BRACE3009747, BRACE3010428, BRACE3011271, BRACE3011421, BRACE3012364, BRACE3013780, BRACE3022769, BRACE3026735, BRACE3031838, BRALZ2011796, BRAMY2003008, BRAMY2005052, BRAMY2019300, BRAMY2019963, BRAMY2036567, BRAMY2037823, BRAMY2040592, BRAMY2047420, BRAMY3002803, BRAMY3004224, BRAMY3005091, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCOC2003213, BRCOC2014033, BRCOC2020142, BRHIP2000920, BRHIP2005719, BRHIP2007741, BRHIP2014228, BRHIP2024146, BRHIP3000339, BRHIP3006683, BRHIP3007586, BRHIP3008405, BRHIP3018797, BRSSN2000684, BRSSN2003086, BRSSN2004496, BRSSN2004719, BRSSN2006892, BRSSN2008549, BRSSN2008797, BRSSN2011262, BRSSN2011738, BRSSN2013874, BRSSN2014299, BRSSN2014424, BRSSN2014556, BRSSN2018581, BRSSN2018925, BRSTN2008052, BRSTN2015015, BRSTN2016470, BRTHA1000311, BRTHA2003461, BRTHA2008335, BRTHA3002427, BRTHA3003490, BRTHA3008520, BRTHA3017848, COLON2001721, CTONG2017500, CTONG2028124, CTONG3000657, CTONG3001123, CTONG3009328, FCBBF2001183, FCBBF3001977, FEBRA2007544, FEBRA2007801, FEBRA2020886, FEBRA2024136, FEBRA2025427, FEBRA2028618, HCASM2007047, HCHON2000244, HCHON2000626, HCHON2001217, HCHON2002676, HCHON2006250, HEART1000074, HHDPC1000118, HLUNG2002465, IMR322000127, IMR322002035, KIDNE2006580, MESAN2006563, MESAN2012054, MESTC1000042, NOVAR2001783, NT2NE2006909, NT2RI2008724, NT2RI2012659, NT2RI2014733, NT2RI3002892, NT2RI3006284, NT2RI3006673, NT2RI3007543, NT2RI3008055, NT2RP7005529, NT2RP7009147, NT2RP7014005, NT2RP7017474, OCBBF2001794, OCBBF2006005, OCBBF2006764, OCBBF2007028, OCBBF2010140, OCBBF2021286, OCBBF2024850, OCBBF2028935, OCBBF2036743, OCBBF2038317, OCBBF3000483, OCBBF3008230, PEBLM2004666, PLACE6001185, PUAEN2005930, PUAEN2006701, PUAEN2007044, PUAEN2009655, SPLEN2028914, SPLEN2031424, SPLEN2031547, SPLEN2034781, SPLEN2036932, SYNOV2014400, SYNOV4002346, SYNOV4002883, SYNOV4007430, SYNOV4008440, TESOP2002451, TESTI4000014, TESTI4000209, TESTI4001100, TESTI4006137, TESTI4008797, TESTI4009286, TESTI4010851, TESTI4013817, TESTI4014694, TESTI4021478, TESTI4022936, TESTI4024420, TESTI4027821, TESTI4037156, THYMU2001090, THYMU2033308, THYMU2035735, THYMU2039315, THYMU3001234, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3001427, TRACH3002192, TRACH3004721, TRACH3005294, TRACH3007479, TRACH3008093, TRACH3009455, UTERU2006115, UTERU2019706, UTERU2023039, UTERU2026203, UTERU3005230, UTERU3007640, UTERU3009871

ADRGL2000042, BLADE2006830, BRACE2003609, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRTHA2018443, BRTHA3003000, CTONG2020374, CTONG2020378, CTONG2024031, FCBBF1000509, FEBRA2001990, FEBRA2006519, FEBRA2028516, HCHON2000743, IMR322001879, NT2RI2009583, OCBBF2008144, PERIC2007068, PUAEN2006335, SPLEN2039379, TESTI2015626, TESTI4001984, TESTI4008058, TESTI4025268, TESTI4032090, THYMU3000360, TLIVE2002046, TRACH3000134, UTERU2021820, UTERU2028734

These genes are involved in Parkinson's disease.

Genes Involved in Short-Term Memory and Dementia

In the brain, the hippocampus is a highly important memory-related area. The hippocampus functions to establish a memory by judging whether acquired information is necessary, and then accumulating the memory in another area of the brain. According to clinical findings, patients can retain a new memory for only about five minutes with an abnormal, or at worst without a hippocampus. Some dementia patients are presumed to have hippocampus abnormalities. Thus, genes whose expression levels differ between tissues of the whole brain and the hippocampus are expected to be involved in memory or dementia. Such genes can be used to elucidate the mechanism underlying memory, and in gene diagnosis. cDNA libraries derived from the hippocampus (BRHIP) and from the whole tissues of a normal brain (BRAWH) were analyzed and compared (Table 6). Genes whose expression levels differed between the two were the 370 clones and 59 clones listed below.

ASTRO1000009, BLADE2001371, BLADE2008398, BNGH42007788, BRACE1000186, BRACE1000258, BRACE1000533, BRACE2005457, BRACE2010489, BRACE2014657, BRACE2015058, BRACE2018762, BRACE2030341, BRACE2035381, BRACE2044286, BRACE2045954, BRACE3000787, BRACE3003192, BRACE3005499, BRACE3007480, BRACE3009237, BRACE3009724, BRACE3009747, BRACE3010428, BRACE3011271, BRACE3011421, BRACE3012364, BRACE3018963, BRACE3022769, BRACE3026735, BRACE3031838, BRALZ2011796, BRAMY2003008, BRAMY2005052, BRAMY2019300, BRAMY2019963, BRAMY2031317, BRAMY2036567, BRAMY2037823, BRAMY2040592, BRAMY2044078, BRAMY3002803, BRAMY3004224, BRAMY3005091, BRAMY3009811, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2020710, BRCAN2028355, BRCOC2003213, BRCOC2014033, BRCOC2020142, BRHIP2000691, BRHIP2000819, BRHIP2000826, BRHIP2000920, BRHIP2001074, BRHIP2001805, BRHIP2001927, BRHIP2002122, BRHIP2002172, BRHIP2002346, BRHIP2003242, BRHIP2003786, BRHIP2003917, BRHIP2004312, BRHIP2004359, BRHIP2004814, BRHIP2004883, BRHIP2005236, BRHIP2005354, BRHIP2005600, BRHIP2005719, BRHIP2005752, BRHIP2005932, BRHIP2006800, BRHIP2007616, BRHIP2007741, BRHIP2009340, BRHIP2009414, BRHIP2009474, BRHIP2013699, BRHIP2014228, BRHIP2021615, BRHIP2022221, BRHIP2024146, BRHIP2024165, BRHIP2026061, BRHIP2026288, BRHIP2029176, BRHIP2029393, BRHIP3000339, BRHIP3000526, BRHIP3001283, BRHIP3006683, BRHIP3007483, BRHIP3007586, BRHIP3008183, BRHIP3008313, BRHIP3008344, BRHIP3008405, BRHIP3008565, BRHIP3008598, BRHIP3008997, BRHIP3009099, BRHIP3009448, BRHIP3011241, BRHIP3013765, BRHIP3013897, BRHIP3015751, BRHIP3016213, BRHIP3018797, BRHIP3020182, BRHIP3024118, BRHIP3024533, BRHIP3024725, BRHIP3025161, BRHIP3025702, BRHIP3026097, BRHIP3027137, BRHIP3027854, BRSSN2000684, BRSSN2004719, BRSSN2008549, BRSSN2011738, BRSSN2014299, BRSTN2008052, BRSTN2015015, BRSTN2016470, BRSTN2018083, BRTHA1000311, BRTHA2002442, BRTHA2008335, BRTHA3000297, BRTHA3001721, BRTHA3002427, BRTHA3003490, BRTHA3005046, BRTHA3008520, BRTHA3008778, BRTHA3009090, BRTHA3015910, BRTHA3017848, COLON2001721, CTONG1000087, CTONG1000088, CTONG1000467, CTONG2000042, CTONG2008233, CTONG2009423, CTONG2017500, CTONG2019788, CTONG2028124, CTONG3000657, CTONG3001123, CTONG3001370, CTONG3002412, CTONG3004072, CTONG3008894, CTONG3009239, CTONG3009328, DFNES2011499, FCBBF2001183, FCBBF3001977, FEBRA2000253, FEBRA2007544, FEBRA2007801, FEBRA2008287, FEBRA2010719, FEBRA2020886, FEBRA2028618, HCASM2007047, HCHON2000028, HCHON2000244, HCHON2000626, HCHON2001217, HCHON2002676, HCHON2005921, HCHON2006250, HEART1000074, HEART2007031, HHDPC1000118, HLUNG2002465, HLUNG2003003, IMR322000127, IMR322001380, IMR322002035, KIDNE2005543, KIDNE2006580, MESAN2006563, MESAN2012054, MESTC1000042, NOVAR2001783, NT2NE2006909, NT2RI2008724, NT2RI2012659, NT2RI2014733, NT2RI2018311, NT2RI3001515, NT2RI3002892, NT2RI3004510, NT2RI3005724, NT2RI3006284, NT2RI3006673, NT2RI3007291, NT2RI3007543, NT2RI3008055, NT2RP7005529, NT2RP7009147, NT2RP7014005, NT2RP7017474, OCBBF2001794, OCBBF2003819, OCBBF2006005, OCBBF2006151, OCBBF2006764, OCBBF2007028, OCBBF2007068, OCBBF2010140, OCBBF2020741, OCBBF2021286, OCBBF2024719, OCBBF2024850, OCBBF2028935, OCBBF2036743, OCBBF2038317, OCBBF3000296, OCBBF3000483, OCBBF3008230, PEBLM2004666, PLACE6001185, PUAEN2005930, PUAEN2006701, PUAEN2007044, PUAEN2009655, SPLEN2010912, SPLEN2012624, SPLEN2028914, SPLEN2031424, SPLEN2031547, SPLEN2034781, SPLEN2036932, SYNOV2014400, SYNOV4002346, SYNOV4002883, SYNOV4007430, SYNOV4008440, TESOP2002451, TESTI2049246, TESTI4000014, TESTI4000209, TESTI4001100, TESTI4006137, TESTI4008797, TESTI4009286, TESTI4010377, TESTI4010851, TESTI4010928, TESTI4011161, TESTI4013817, TESTI4014159, TESTI4014694, TESTI4014818, TESTI4021478, TESTI4022936, TESTI4024420, TESTI4027821, TESTI4037156, THYMU2001090, THYMU2023967, THYMU2025707, THYMU2031341, THYMU2033308, THYMU2035735, THYMU2037226, THYMU2039315, THYMU3001234, THYMU3001379, THYMU3004835, THYMU3007137, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3000014, TRACH3001427, TRACH3002192, TRACH3004721, TRACH3005294, TRACH3007479, TRACH3008093, TRACH3009455, TUTER1000122, TUTER2000904, UTERU2004929, UTERU2006115, UTERU2019706, UTERU2021163, UTERU2023039, UTERU2026203, UTERU2030213, UTERU3001572, UTERU3003135, UTERU3005230, UTERU3007640, UTERU3009259, UTERU3009871

ADRGL2000042, BLADE2006830, BRACE2003609, BRAMY3004800, BRAWH1000369, BRAWH2006207, BRAWH2006395, BRAWH2008993, BRAWH2009393, BRAWH2010552, BRAWH3007441, BRAWH3009017, BRHIP2002722, BRHIP2003272, BRHIP2005271, BRHIP2005724, BRHIP2006617, BRHIP2008389, BRHIP2012360, BRHIP2017553, BRHIP2026877, BRHIP3000017, BRHIP3000240, BRHIP3008314, BRHIP3026052, BRTHA2018443, BRTHA3003000, CTONG2020374, CTONG2020378, CTONG2024031, CTONG3004726, FCBBF1000509, FEBRA2001990, FEBRA2006519, FEBRA2028516, HCHON2000743, IMR322001879, NT2RI2009583, OCBBF2006987, OCBBF2008144, OCBBF2030116, PERIC2007068, PUAEN2006335, SPLEN2039379, TESTI2015626, TESTI4000214, TESTI4001984, TESTI4008058, TESTI4013894, TESTI4025268, TESTI4025547, TESTI4026207, TESTI4032090, THYMU3000360, TLIVE2002046, TRACH3000134, UTERU2008040, UTERU2021820, UTERU2028734

These genes are involved in memory and dementia.

Genes Involved in Equilibrium Sense and Movement Function

The cerebellum is the center of equilibrium sense, muscular movement, and motor learning. This area is thought to be involved in motor control, and smooth movements are achieved unconsciously due to cerebellum action. Recent studies have elucidated that the cerebellum participates in not only simple movements but also in establishing higher-order movements such as reading and writing. Thus, genes whose expression levels differ between tissues of the whole brain and the cerebellum are expected to be involved in equilibrium sense or motor function, which can be useful for elucidating the molecular mechanism controlled by the brain. cDNA libraries derived from the cerebellum (BRACE) and from the whole tissues of a normal brain (BRAWH) were analyzed and compared (Table 7). Genes whose expression levels differed between the two were the 488 clones and 66 clones listed below.

ADRGL2009146, ADRGL2012038, ASTRO1000009, ASTRO2003960, BLADE1000176, BLADE2004089, BLADE2008398, BRACE1000186, BRACE1000258, BRACE1000533, BRACE1000572, BRACE2003639, BRACE2005457, BRACE2006319, BRACE2008594, BRACE2010489, BRACE2011747, BRACE2014306, BRACE2014475, BRACE2014657, BRACE2015058, BRACE2015314, BRACE2016981, BRACE2018762, BRACE2024627, BRACE2026836, BRACE2027258, BRACE2027970, BRACE2028970, BRACE2029112, BRACE2029849, BRACE2030326, BRACE2030341, BRACE2030884, BRACE2031154, BRACE2031389, BRACE2031527, BRACE2031531, BRACE2031899, BRACE2032044, BRACE2032329, BRACE2032385, BRACE2032538, BRACE2032823, BRACE2033720, BRACE2035381, BRACE2035441, BRACE2036005, BRACE2036096, BRACE2036830, BRACE2036834, BRACE2037847, BRACE2038114, BRACE2038329, BRACE2038551, BRACE2039249, BRACE2039327, BRACE2039475, BRACE2039734, BRACE2040138, BRACE2040325, BRACE2041009, BRACE2041200, BRACE2041264, BRACE2042550, BRACE2043142, BRACE2043248, BRACE2043349, BRACE2043665, BRACE2044286, BRACE2044816, BRACE2044949, BRACE2045300, BRACE2045428, BRACE2045596, BRACE2045772, BRACE2045947, BRACE2045954, BRACE2046251, BRACE2046295, BRACE2047011, BRACE2047350, BRACE2047377, BRACE2047385, BRACE3000071, BRACE3000697, BRACE3000787, BRACE3000840, BRACE3000973, BRACE3001002, BRACE3001217, BRACE3001391, BRACE3001595, BRACE3001754, BRACE3002298, BRACE3002390, BRACE3002508, BRACE3003004, BRACE3003192, BRACE3003595, BRACE3003698, BRACE3004058, BRACE3004113, BRACE3004150, BRACE3004358, BRACE3004435, BRACE3004772, BRACE3004783, BRACE3004843, BRACE3004880, BRACE3005145, BRACE3005225, BRACE3005430, BRACE3005499, BRACE3006185, BRACE3006226, BRACE3006462, BRACE3006872, BRACE3007322, BRACE3007472, BRACE3007480, BRACE3007559, BRACE3007625, BRACE3007642, BRACE3007767, BRACE3008036, BRACE3008092, BRACE3008137, BRACE3008384, BRACE3008720, BRACE3008772, BRACE3009090, BRACE3009237, BRACE3009297, BRACE3009377, BRACE3009574, BRACE3009701, BRACE3009708, BRACE3009724, BRACE3009747, BRACE3010397, BRACE3010428, BRACE3011271, BRACE3011421, BRACE3011505, BRACE3012364, BRACE3012930, BRACE3013119, BRACE3013576, BRACE3013740, BRACE3013780, BRACE3014005, BRACE3014068, BRACE3014231, BRACE3014317, BRACE3014807, BRACE3015027, BRACE3015121, BRACE3015262, BRACE3015521, BRACE3015894, BRACE3016884, BRACE3018308, BRACE3018963, BRACE3019055, BRACE3019084, BRACE3020194, BRACE3020286, BRACE3020594, BRACE3022769, BRACE3023912, BRACE3024073, BRACE3024659, BRACE3024662, BRACE3025153, BRACE3025457, BRACE3025531, BRACE3025630, BRACE3026008, BRACE3026075, BRACE3026735, BRACE3027242, BRACE3027326, BRACE3027478, BRACE3030103, BRACE3031838, BRACE3032983, BRACE3040856, BRACE3045033, BRALZ2011796, BRAMY2003008, BRAMY2005052, BRAMY2019300, BRAMY2019963, BRAMY2020058, BRAMY2030098, BRAMY2031317, BRAMY2036567, BRAMY2037823, BRAMY2039872, BRAMY2040592, BRAMY2044078, BRAMY2047420, BRAMY3002620, BRAMY3002803, BRAMY3004224, BRAMY3005091, BRAMY3005932, BRAMY4000229, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2009432, BRCAN2010376, BRCAN2015371, BRCAN2020710, BRCOC2003213, BRCOC2007034, BRCOC2014033, BRCOC2020142, BRHIP2000920, BRHIP2004359, BRHIP2005719, BRHIP2005752, BRHIP2007741, BRHIP2013699, BRHIP2014228, BRHIP2024146, BRHIP3000339, BRHIP3006683, BRHIP3007586, BRHIP3008313, BRHIP3008405, BRHIP3018797, BRSSN2000684, BRSSN2006892, BRSSN2011262, BRSSN2011738, BRSSN2014299, BRSTN2008052, BRSTN2010750, BRSTN2015015, BRSTN2016470, BRTHA1000311, BRTHA2008335, BRTHA2008955, BRTHA2011194, BRTHA3001721, BRTHA3002427, BRTHA3003490, BRTHA3008520, BRTHA3009090, BRTHA3017848, COLON2001721, CTONG2008233, CTONG2017500, CTONG2028124, CTONG3000657, CTONG3001123, CTONG3005813, CTONG3008894, CTONG3009328, DFNES2011499, FCBBF2001183, FCBBF3001977, FEBRA2006396, FEBRA2007544, FEBRA2007708, FEBRA2007801, FEBRA2008287, FEBRA2020886, FEBRA2021966, FEBRA2026984, FEBRA2028618, HCASM2007047, HCHON2000244, HCHON2000626, HCHON2001217, HCHON2002676, HCHON2005921, HCHON2006250, HEART1000074, HHDPC1000118, HLUNG2002465, IMR322000127, IMR322001380, IMR322002035, KIDNE2000665, KIDNE2006580, MESAN2006563, MESAN2012054, MESTC1000042, NB9N41000340, NESOP2001752, NOVAR2001783, NT2NE2006909, NT2RI2005166, NT2RI2008724, NT2RI2012659, NT2RI2014733, NT2RI2019751, NT2RI3002892, NT2RI3003382, NT2RI3004510, NT2RI3005724, NT2RI3006284, NT2RI3006673, NT2RI3007291, NT2RI3007543, NT2RI3008055, NT2RP7004123, NT2RP7005529, NT2RP7009147, NT2RP7010599, NT2RP7014005, NT2RP7017474, NTONG2005969, OCBBF2001794, OCBBF2003819, OCBBF2006005, OCBBF2006151, OCBBF2006764, OCBBF2007028, OCBBF2010140, OCBBF2020343, OCBBF2020741, OCBBF2021286, OCBBF2022351, OCBBF2024850, OCBBF2025527, OCBBF2028935, OCBBF2036743, OCBBF2038317, OCBBF3000483, OCBBF3007516, OCBBF3008230, PEBLM2004666, PERIC2000889, PLACE6001185, PUAEN2002489, PUAEN2005930, PUAEN2006701, PUAEN2007044, PUAEN2009655, SPLEN2010912, SPLEN2012624, SPLEN2027268, SPLEN2028914, SPLEN2031424, SPLEN2031547, SPLEN2034781, SPLEN2036932, SPLEN2037194, SYNOV2014400, SYNOV4002346, SYNOV4002883, SYNOV4007430, SYNOV4007671, SYNOV4008440, TESOP2002273, TESOP2002451, TESOP2002950, TESTI1000330, TESTI4000014, TESTI4000209, TESTI4000349, TESTI4001100, TESTI4001561, TESTI4006137, TESTI4008797, TESTI4009286, TESTI4010851, TESTI4011161, TESTI4013675, TESTI4013817, TESTI4014159, TESTI4014306, TESTI4014694, TESTI4021478, TESTI4022936, TESTI4024420, TESTI4027821, TESTI4037156, TESTI4046819, THYMU2001090, THYMU2016523, THYMU2023967, THYMU2030264, THYMU2033308, THYMU2035735, THYMU2039315, THYMU2039780, THYMU3001083, THYMU3001234, THYMU3003309, THYMU3006485, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3001427, TRACH3002192, TRACH3004721, TRACH3005294, TRACH3006038, TRACH3006412, TRACH3007479, TRACH3008093, TRACH3009455, TUTER2000904, UTERU2002410, UTERU2006115, UTERU2007520, UTERU2019706, UTERU2023039, UTERU2026203, UTERU3000226, UTERU3001572, UTERU3005230, UTERU3005460, UTERU3005970, UTERU3006308, UTERU3007419, UTERU3007640, UTERU3007913, UTERU3009871

ADRGL2000042, BLADE2006830, BRACE2002589, BRACE2003609, BRACE2009318, BRACE2011677, BRACE2029396, BRACE2037299, BRACE2039823, BRACE2039832, BRACE2043105, BRACE3001058, BRACE3001113, BRACE3003026, BRACE3003053, BRACE3009127, BRACE3010076, BRACE3015829, BRACE3021148, BRAMY3004800, BRAWH1000369, BRAWH2006207, BRAWH2006395, BRAWH2008993, BRAWH2009393, BRAWH2010552, BRAWH3007441, BRAWH3009017, BRCOC2019841, BRHIP2005271, BRHIP3000017, BRHIP3000240, BRTHA2018443, BRTHA3003000, CTONG2020374, CTONG2020378, CTONG2024031, FCBBF1000509, FEBRA2001990, FEBRA2006519, FEBRA2028516, HCHON2000743, IMR322001879, NT2RI2009583, NT2RP8000521, OCBBF2008144, OCBBF2011669, PERIC2007068, PUAEN2006335, SPLEN2039379, SYNOV2021953, TESTI2015626, TESTI4001984, TESTI4008058, TESTI4013894, TESTI4025268, TESTI4032090, THYMU2004284, THYMU2040925, THYMU3000360, TLIVE2002046, TRACH3000134, UTERU2008040, UTERU2011220, UTERU2021820, UTERU2028734

These genes are involved in equilibrium sense or motor function.

Genes Involved in Signaling from Sensory Organs

The thalamus is an area which comprises many neurons strongly connected to the cerebrum, and which transmits sensory information from the spinal cord or such to the responsible area of the cerebrum. The thalamus also controls the direction of movement from the cerebrum. For example, the thalamus resolves vision into the elements of size, shape, and color, and resolves sound into volume and sweetness or harshness to the ear, and then transmits this information to the sensory area of the cerebral cortex. Thus, genes whose expression levels differ between tissues of the whole brain and the thalamus are expected to be involved in signaling from sensory organs. These genes can be used to elucidate the molecular mechanism underlying signaling controlled by the brain. cDNA libraries derived from the thalamus (BRTHA) and from whole tissues of a normal brain (BRAWH) were analyzed and compared (Table 8). Genes whose expression levels differed between the two were the 412 clones and 56 clones listed below.

ASTRO1000009, ASTRO3000482, BLADE2008398, BRACE1000186, BRACE1000258, BRACE1000533, BRACE2005457, BRACE2010489, BRACE2014306, BRACE2014657, BRACE2015058, BRACE2031154, BRACE2035381, BRACE2044286, BRACE2045954, BRACE3000787, BRACE3003192, BRACE3005499, BRACE3007480, BRACE3008384, BRACE3009237, BRACE3009724, BRACE3009747, BRACE3010397, BRACE3010428, BRACE3011271, BRACE3011421, BRACE3012364, BRACE3022769, BRACE3026735, BRACE3027478, BRACE3031838, BRALZ2011796, BRAMY2003008, BRAMY2005052, BRAMY2019300, BRAMY2019963, BRAMY2028914, BRAMY2031317, BRAMY2036567, BRAMY2037823, BRAMY2040592, BRAMY2044078, BRAMY3002803, BRAMY3004224, BRAMY3005091, BRAMY4000229, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2006297, BRCOC2003213, BRCOC2014033, BRCOC2020142, BRHIP2000819, BRHIP2000920, BRHIP2005719, BRHIP2007741, BRHIP2009474, BRHIP2013699, BRHIP2014228, BRHIP2022221, BRHIP2024146, BRHIP3000339, BRHIP3006683, BRHIP3007586, BRHIP3008405, BRHIP3018797, BRSSN2000684, BRSSN2008549, BRSSN2008797, BRSSN2011738, BRSSN2014299, BRSTN2004863, BRSTN2008052, BRSTN2015015, BRSTN2016470, BRTHA1000311, BRTHA2000855, BRTHA2001462, BRTHA2002115, BRTHA2002281, BRTHA2002376, BRTHA2002442, BRTHA2002493, BRTHA2002608, BRTHA2002808, BRTHA2003030, BRTHA2003110, BRTHA2003116, BRTHA2003461, BRTHA2004821, BRTHA2004978, BRTHA2005579, BRTHA2005956, BRTHA2006075, BRTHA2006146, BRTHA2006194, BRTHA2007122, BRTHA2007422, BRTHA2007603, BRTHA2008316, BRTHA2008335, BRTHA2008527, BRTHA2008535, BRTHA2008955, BRTHA2009311, BRTHA2009846, BRTHA2009972, BRTHA2010073, BRTHA2010608, BRTHA2010884, BRTHA2010907, BRTHA2011194, BRTHA2011351, BRTHA2011500, BRTHA2011641, BRTHA2012392, BRTHA2012562, BRTHA2012980, BRTHA2013262, BRTHA2013460, BRTHA2013707, BRTHA2014792, BRTHA2014828, BRTHA2015406, BRTHA2015478, BRTHA2015696, BRTHA2015878, BRTHA2016215, BRTHA2016496, BRTHA2016543, BRTHA2017353, BRTHA2017985, BRTHA2018165, BRTHA2018344, BRTHA2018591, BRTHA2018624, BRTHA2018707, BRTHA2019014, BRTHA2019022, BRTHA2019048, BRTHA3000273, BRTHA3000297, BRTHA3000633, BRTHA3001721, BRTHA3002401, BRTHA3002427, BRTHA3002933, BRTHA3003074, BRTHA3003343, BRTHA3003449, BRTHA3003474, BRTHA3003490, BRTHA3004475, BRTHA3005046, BRTHA3006856, BRTHA3007113, BRTHA3007148, BRTHA3007319, BRTHA3007769, BRTHA3008143, BRTHA3008310, BRTHA3008386, BRTHA3008520, BRTHA3008778, BRTHA3009037, BRTHA3009090, BRTHA3009291, BRTHA3010366, BRTHA3013884, BRTHA3015815, BRTHA3015910, BRTHA3016845, BRTHA3016917, BRTHA3017047, BRTHA3017589, BRTHA3017848, BRTHA3018514, BRTHA3018617, BRTHA3018656, BRTHA3019105, COLON2001721, CTONG1000087, CTONG2008233, CTONG2017500, CTONG2019788, CTONG2023021, CTONG2028124, CTONG3000657, CTONG3001123, CTONG3008894, CTONG3009028, CTONG3009239, CTONG3009328, FCBBF2001183, FCBBF3001977, FCBBF3021576, FEBRA2007544, FEBRA2007801, FEBRA2008287, FEBRA2008360, FEBRA2020886, FEBRA2028618, HCASM2007047, HCHON2000028, HCHON2000212, HCHON2000244, HCHON2000626, HCHON2001084, HCHON2001217, HCHON2002676, HCHON2005921, HCHON2006250, HEART1000074, HEART2007031, HHDPC1000118, HLUNG2001996, HLUNG2002465, IMR322000127, IMR322001380, IMR322002035, KIDNE2002252, KIDNE2005543, KIDNE2006580, KIDNE2011314, MESAN2006563, MESAN2012054, MESTC1000042, NOVAR2001783, NT2NE2006909, NT2RI2008724, NT2RI2012659, NT2RI2014733, NT2RI3002842, NT2RI3002892, NT2RI3005403, NT2RI3006284, NT2RI3006673, NT2RI3007543, NT2RI3008055, NT2RP7004123, NT2RP7005529, NT2RP7009147, NT2RP7014005, NT2RP7017474, NTONG2005969, NTONG2008088, OCBBF2001794, OCBBF2006005, OCBBF2006764, OCBBF2007028, OCBBF2010140, OCBBF2020639, OCBBF2021286, OCBBF2024719, OCBBF2024850, OCBBF2028935, OCBBF2036743, OCBBF2038317, OCBBF3000483, OCBBF3008230, PEBLM2004666, PLACE6001185, PUAEN2002489, PUAEN2005930, PUAEN2006701, PUAEN2007044, PUAEN2009655, RECTM2001347, SKMUS2000757, SPLEN2006122, SPLEN2010912, SPLEN2025491, SPLEN2028914, SPLEN2031424, SPLEN2031547, SPLEN2032154, SPLEN2034781, SPLEN2036821, SPLEN2036932, SYNOV1000374, SYNOV2014400, SYNOV4002346, SYNOV4002883, SYNOV4007430, SYNOV4007671, SYNOV4008440, TESOP2002451, TESTI2049246, TESTI4000014, TESTI4000209, TESTI4001100, TESTI4002290, TESTI4006137, TESTI4008797, TESTI4009286, TESTI4010851, TESTI4012702, TESTI4013817, TESTI4014159, TESTI4014694, TESTI4021478, TESTI4022936, TESTI4024420, TESTI4027821, TESTI4037156, THYMU2001090, THYMU2025707, THYMU2032825, THYMU2033308, THYMU2033787, THYMU2035735, THYMU2039315, THYMU2040975, THYMU3001234, THYMU3001379, THYMU3004835, THYMU3008171, TKIDN2009641, TKIDN2009889, TKIDN2015788, TLIVE2001327, TRACH1000205, TRACH2001549, TRACH2005811, TRACH2006049, TRACH2007834, TRACH2008300, TRACH2023299, TRACH2025535, TRACH3001427, TRACH3002192, TRACH3004068, TRACH3004721, TRACH3005294, TRACH3007479, TRACH3008093, TRACH3009455, TSTOM1000135, TUTER2000904, UTERU2002410, UTERU2006115, UTERU2019706, UTERU2019940, UTERU2023039, UTERU2023175, UTERU2026203, UTERU2030280, UTERU3000899, UTERU3001571, UTERU3001572, UTERU3004709, UTERU3005230, UTERU3005907, UTERU3007640, UTERU3009871

ADRGL2000042, BLADE2006830, BRACE2003609, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRTHA2002133, BRTHA2002702, BRTHA2007060, BRTHA2010033, BRTHA2011321, BRTHA2013426, BRTHA2013610, BRTHA2016318, BRTHA2017364, BRTHA2017972, BRTHA2018011, BRTHA2018443, BRTHA3000296, BRTHA3003000, BRTHA3008826, CTONG2008721, CTONG2020374, CTONG2020378, CTONG2024031, FCBBF1000509, FEBRA2001990, FEBRA2006519, FEBRA2028516, HCHON2000743, HSYRA2005628, IMR322001879, NT2RI2009583, OCBBF2008144, PERIC2007068, PUAEN2006335, SPLEN2016932, SPLEN2039379, SYNOV2006620, TESTI4001984, TESTI4008058, TESTI4025268, TESTI4032090, THYMU3000360, TLIVE2002046, TRACH3000134, UTERU2021820, UTERU2028734

These genes are involved in signaling from sensory organs.

Genes Involved in Emotional Reaction

The amygdala is the center of emotion in the brain. Information passing through the amygdala induces an emotional reaction, for example, panic or fear. When a strong fear reaction is produced due to the emotional evaluation of stimulus in the amygdala, the amygdala transmits an alert signal to each area of the brain. This results in various reactions such as sweating palms, palpitation, elevated blood pressure, and rapid secretion of adrenaline. In other words, the amygdala transmits signals which cause the body to be on the alert and is a tissue involved in a kind of defense instinct. Thus, genes whose expression levels differ between tissues of the whole brain and the amygdala are expected to be involved in emotional reaction. Such genes can be used to elucidate the molecular mechanism underlying emotional reaction, fear, or panic. cDNA libraries derived from the amygdale (BRAMY) and from whole tissues of a normal brain (BRAWH) were analyzed and compared (Table 9). Genes whose expression levels differed between the two were the 383 clones and 55 clones listed below.

ASTRO1000009, BLADE2008398, BRACE1000186, BRACE1000258, BRACE1000533, BRACE2005457, BRACE2006319, BRACE2010489, BRACE2014657, BRACE2015058, BRACE2027258, BRACE2030341, BRACE2031389, BRACE2035381, BRACE2044286, BRACE2045954, BRACE3000787, BRACE3000973, BRACE3003192, BRACE3005499, BRACE3007480, BRACE3008036, BRACE3009237, BRACE3009724, BRACE3009747, BRACE3010428, BRACE3011271, BRACE3011421, BRACE3012364, BRACE3013780, BRACE3022769, BRACE3026735, BRACE3027478, BRACE3031838, BRALZ2011796, BRAMY2001473, BRAMY2003008, BRAMY2004771, BRAMY2005052, BRAMY2017528, BRAMY2019300, BRAMY2019963, BRAMY2019985, BRAMY2020058, BRAMY2020270, BRAMY2021498, BRAMY2028856, BRAMY2028914, BRAMY2029602, BRAMY2030098, BRAMY2030109, BRAMY2030702, BRAMY2030703, BRAMY2030799, BRAMY2031317, BRAMY2031377, BRAMY2031442, BRAMY2032014, BRAMY2032242, BRAMY2032317, BRAMY2033003, BRAMY2033116, BRAMY2033267, BRAMY2033594, BRAMY2034185, BRAMY2034920, BRAMY2034993, BRAMY2036387, BRAMY2036396, BRAMY2036567, BRAMY2036699, BRAMY2036913, BRAMY2037823, BRAMY2038100, BRAMY2038484, BRAMY2038846, BRAMY2038904, BRAMY2039872, BRAMY2040478, BRAMY2040592, BRAMY2041261, BRAMY2041378, BRAMY2041542, BRAMY2042612, BRAMY2042641, BRAMY2042760, BRAMY2042918, BRAMY2044078, BRAMY2044246, BRAMY2045036, BRAMY2046478, BRAMY2046742, BRAMY2046989, BRAMY2047169, BRAMY2047420, BRAMY2047676, BRAMY2047746, BRAMY2047751, BRAMY2047765, BRAMY2047884, BRAMY3000206, BRAMY3000213, BRAMY3001401, BRAMY3001794, BRAMY3002312, BRAMY3002620, BRAMY3002803, BRAMY3002805, BRAMY3004224, BRAMY3004672, BRAMY3004900, BRAMY3004919, BRAMY3005091, BRAMY3005932, BRAMY3006297, BRAMY3007206, BRAMY3007609, BRAMY3008466, BRAMY3008505, BRAMY3008650, BRAMY3009811, BRAMY3010411, BRAMY4000095, BRAMY4000229, BRAMY4000277, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2014881, BRCAN2017717, BRCOC2000670, BRCOC2003213, BRCOC2014033, BRCOC2020142, BRHIP2000920, BRHIP2005719, BRHIP2007741, BRHIP2014228, BRHIP2024146, BRHIP2026061, BRHIP3000339, BRHIP3001283, BRHIP3006683, BRHIP3007586, BRHIP3008405, BRHIP3018797, BRSSN2000684, BRSSN2004496, BRSSN2011738, BRSSN2014299, BRSTN2008052, BRSTN2010750, BRSTN2015015, BRSTN2016470, BRTHA1000311, BRTHA2008335, BRTHA2011641, BRTHA3001721, BRTHA3002427, BRTHA3003490, BRTHA3004475, BRTHA3008520, BRTHA3009090, BRTHA3017848, COLON2001721, CTONG1000087, CTONG2008233, CTONG2017500, CTONG2028124, CTONG3000657, CTONG3001123, CTONG3008894, CTONG3009239, CTONG3009328, FCBBF2001183, FCBBF3001977, FEBRA2007544, FEBRA2007801, FEBRA2008287, FEBRA2010719, FEBRA2020886, FEBRA2025427, FEBRA2028618, HCASM2007047, HCHON2000244, HCHON2000626, HCHON2001217, HCHON2002676, HCHON2006250, HCHON2008112, HEART1000074, HHDPC1000118, HLUNG2002465, HSYRA2009075, IMR322000127, IMR322001380, IMR322002035, KIDNE2000665, KIDNE2006580, MESAN2006563, MESAN2012054, MESAN2015515, MESTC1000042, NOVAR2001783, NT2NE2005890, NT2NE2006909, NT2RI2008724, NT2RI2012659, NT2RI2014733, NT2RI3001515, NT2RI3002892, NT2RI3005724, NT2RI3006284, NT2RI3006673, NT2RI3007543, NT2RI3008055, NT2RP7005529, NT2RP7009147, NT2RP7014005, NT2RP7017474, NTONG2005969, OCBBF1000254, OCBBF2001794, OCBBF2006005, OCBBF2006764, OCBBF2007028, OCBBF2007114, OCBBF2010140, OCBBF2021286, OCBBF2023162, OCBBF2024850, OCBBF2028935, OCBBF2035214, OCBBF2036743, OCBBF2038317, OCBBF3000483, OCBBF3008230, PEBLM2004666, PERIC2000889, PERIC2003720, PLACE6001185, PUAEN2005930, PUAEN2006701, PUAEN2007044, PUAEN2009174, PUAEN2009655, SKNMC2002402, SKNSH2000482, SPLEN2001599, SPLEN2002467, SPLEN2028914, SPLEN2029912, SPLEN2031424, SPLEN2031547, SPLEN2034781, SPLEN2036932, SPLEN2038345, SYNOV2014400, SYNOV4002346, SYNOV4002883, SYNOV4007430, SYNOV4007671, SYNOV4008440, TESOP2002451, TESTI2009474, TESTI4000014, TESTI4000209, TESTI4001100, TESTI4006137, TESTI4008797, TESTI4009286, TESTI4010851, TESTI4013817, TESTI4014159, TESTI4014694, TESTI4021478, TESTI4022936, TESTI4024420, TESTI4027821, TESTI4029836, TESTI4037156, TESTI4037188, THYMU2001090, THYMU2014353, THYMU2033308, THYMU2035735, THYMU2037226, THYMU2039315, THYMU3001234, THYMU3001379, THYMU3004835, THYMU3008171, TKIDN2009641, TKIDN2009889, TKIDN2015788, TLIVE2004320, TRACH1000205, TRACH2001549, TRACH2001684, TRACH2005811, TRACH2006049, TRACH2007834, TRACH2008300, TRACH2025344, TRACH2025535, TRACH2025911, TRACH3001427, TRACH3002192, TRACH3004068, TRACH3004721, TRACH3005294, TRACH3007479, TRACH3008093, TRACH3009455, TUTER2000904, UTERU2002410, UTERU2004929, UTERU2006115, UTERU2007520, UTERU2019706, UTERU2023039, UTERU2026203, UTERU3001572, UTERU3001766, UTERU3005230, UTERU3007640, UTERU3009517, UTERU3009871

ADRGL2000042, BLADE2006830, BRACE2003609, BRACE2039823, BRAMY2019111, BRAMY2035070, BRAMY2035449, BRAMY2035718, BRAMY2038516, BRAMY2039341, BRAMY2040159, BRAMY2041434, BRAMY2045471, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRTHA2018443, BRTHA3003000, CTONG2020374, CTONG2020378, CTONG2024031, FCBBF1000509, FEBRA2001990, FEBRA2006519, FEBRA2028516, HCHON2000508, HCHON2000743, IMR322001879, NT2RI2009583, OCBBF2008144, PERIC2007068, PUAEN2006335, SPLEN2039379, TESTI2015626, TESTI2026647, TESTI4001984, TESTI4008058, TESTI4013894, TESTI4025268, TESTI4032090, THYMU3000360, TKIDN2018926, TLIVE2002046, TRACH3000134, UTERU2008040, UTERU2021820, UTERU2028734

These genes are involved in emotional reaction.

Cancer-Related Genes

Cancer tissues are assumed to express a distinct set of genes distinct from normal tissues, and thus expression of these genes can contribute to carcinogenesis in tissues and cells. Thus, genes whose expression patterns in cancer tissues differ from those in normal tissues are cancer-related genes. A search was carried out for genes whose expression levels in cancer tissues differed from those in normal tissues.

The result of comparative analysis of cDNA libraries derived from breast tumor (TBAES) and normal breast (BEAST) (Table 10) showed that the genes whose expression levels differed between the two were the 35 and four clones listed below.

ASTRO2002842, BRACE3016884, BRSSN2011262, BRTHA2008335, HCHON2000244, HCHON2006250, HEART1000010, MESAN2012054, NT2RP7000466, NT2RP7009147, OCBBF2021020, PEBLM2002749, PEBLM2004666, SPLEN2001599, SPLEN2031547, STOMA1000189, TBAES2001171, TBAES2001220, TBAES2001229, TBAES2001258, TBAES2001492, TBAES2001751, TBAES2002197, TBAES2003550, TBAES2004055, TBAES2005157, TBAES2005543, TBAES2006568, TBAES2007964, TESTI4000014, TESTI4037156, TRACH3002192, TRACH3004068, TSTOM2000553, UTERU2002410 BRAWH2006395, NT2RI2009583, STOMA2004893, TBAES2000932

The result of comparative analysis of cDNA libraries derived from cervical tumor (TCERX) and normal cervical duct (CERVX) (Table 11) showed that the genes whose expression levels differed between the two were twelve and two clones listed below.

BLADE2007666, BRAMY2047420, BRCAN2007409, BRSTN2016470, CERVX1000042, CERVX2002006, MESAN2006563, PROST2018090, TCERX2000613, TESTI4037156, THYMU2031341, UTERU2004688 CERVX2002013, NT2RI2009583

The result of comparative analysis of cDNA libraries derived from colon tumor (TCOLN) and normal colon (COLON) (Table 12) showed that the genes whose expression levels differed between the two were the 24 and four clones listed below.

BRACE3015027, BRAMY2040592, BRSTN2016470, COLON1000030, COLON2000470, COLON2000568, COLON2001721, COLON2002443, COLON2002520, COLON2003043, COLON2004478, COLON2005126, COLON2005772, COLON2006282, COLON2009499, OCBBF2028935, PLACE7000514, RECTM2000433, SYNOV4007671, TCOLN2002278, TESTI2052693, TESTI4037156, THYMU2031368, TRACH2025535 CTONG1000113, NT2RI2009583, NT2RI2018448, TESTI2015626

The result of comparative analysis of cDNA libraries derived from esophageal tumor (TESOP) and normal esophagus (NESOP) (Table 13) showed that the genes whose expression levels differed between the two were the 56 and ten clones listed below.

BRACE2030341, BRAMY2047420, BRHIP2003917, BRTHA2003461, CTONG2013178, D3OST3000169, FEBRA2025427, HCHON2000244, HHDPC1000118, NESOP2000744, NESOP2001433, NESOP2001656, NESOP2001694, NESOP2001752, NESOP2002738, NT2RI3006284, NT2RP7009147, PLACE6019932, SYNOV2005216, TESOP1000127, TESOP2000801, TESOP2001122, TESOP2001166, TESOP2001345, TESOP2001605, TESOP2001818, TESOP2001849, TESOP2001865, TESOP2001953, TESOP2002273, TESOP2002451, TESOP2002489, TESOP2002539, TESOP2002950, TESOP2003273, TESOP2003753, TESOP2004114, TESOP2005285, TESOP2005485, TESOP2005579, TESOP2006041, TESOP2006060, TESOP2006068, TESOP2006670, TESOP2006746, TESOP2007052, TESOP2007262, TESOP2007636, TESOP2007688, TESOP2009121, TESOP2009555, TESTI4009286, TESTI4010851, THYMU2040975, TRACH2005811, UTERU2023175 CTONG2016942, NT2RI2009583, TESOP2000390, TESOP2001796, TESOP2005199, TESOP2006398, TESOP2006865, TESOP2007384, TESTI2015626, TRACH2000862

The result of comparative analysis of cDNA libraries derived from kidney tumor (TKIDN) and normal kidney (KIDNE) (Table 14) showed that the genes whose expression levels differed between the two were the 96 and 13 clones listed below.

ASTRO2018373, BRACE1000186, BRACE2014306, BRACE2015058, BRACE2016981, BRACE2043665, BRACE3008036, BRACE3010428, BRACE3022769, BRAMY2019963, BRAMY2044078, BRAWH1000127, BRAWH2001395, BRAWH2001671, BRAWH2013294, BRAWH2014645, BRHIP2024146, BRHIP3000339, BRSSN2000684, BRSSN2004719, BRSSN2018581, BRSTN2016470, BRTHA1000311, BRTHA3002427, CTONG1000087, CTONG2028124, CTONG3000657, CTONG3008894, FCBBF2001183, FEBRA2008287, HCASM2001301, HCHON2000028, HCHON2000244, HEART1000074, HHDPC1000118, HSYRA2008376, KIDNE1000064, KIDNE2000665, KIDNE2000722, KIDNE2000832, KIDNE2000846, KIDNE2001361, KIDNE2001847, KIDNE2002252, KIDNE2002991, KIDNE2003837, KIDNE2005543, KIDNE2006580, KIDNE2010264, KIDNE2011314, KIDNE2011532, KIDNE2011635, KIDNE2012945, KIDNE2013095, NESOP2001656, NTONG2005969, PEBLM2004666, SKMUS2000757, STOMA1000189, SYNOV4007671, TBAES2001258, TESTI4000014, TESTI4001100, TESTI4012702, TESTI4046819, THYMU2032014, TKIDN2000701, TKIDN2002424, TKIDN2002632, TKIDN2003044, TKIDN2004386, TKIDN2005934, TKIDN2005947, TKIDN2006525, TKIDN2006852, TKIDN2007667, TKIDN2009092, TKIDN2009641, TKIDN2009889, TKIDN2010934, TKIDN2012824, TKIDN2013287, TKIDN2014757, TKIDN2014771, TKIDN2015263, TKIDN2015788, TKIDN2016309, TKIDN2019116, TRACH2001443, TRACH2001684, TRACH2007834, TRACH2008300, TRACH3001427, UTERU2002410, UTERU2023175, UTERU3001572

BLADE2006830, BRALZ2017844, CTONG2028758, FCBBF1000509, FEBRA2001990, FEBRA2028516, HCHON2000508, MESAN2005303, NT2RI2009583, TESTI2015626, TKIDN2008778, TKIDN2012771, TKIDN2018926

The result of comparative analysis of cDNA libraries derived from liver tumor (TLIVE) and normal liver (LIVER) (Table 15) showed that the genes whose expression levels differed between the two were the 35 and six clones listed below.

BRCAN2018935, BRSTN2016470, BRTHA2012980, BRTHA3002427, CTONG2028124, LIVER2007415, NT2RI2008724, SPLEN2012624, SPLEN2033098, TESOP2002451, TLIVE2000023, TLIVE2001327, TLIVE2001828, TLIVE2001927, TLIVE2002336, TLIVE2002338, TLIVE2002690, TLIVE2003197, TLIVE2003225, TLIVE2003381, TLIVE2003970, TLIVE2004110, TLIVE2004320, TLIVE2004601, TLIVE2005180, TLIVE2006236, TLIVE2006529, TLIVE2007132, TLIVE2007528, TLIVE2007816, TLIVE2008083, TLIVE2008229, TLIVE2009541, UTERU2002410, UTERU2005621

LIVER2000247, NT2RI2009583, TESTI2015626, TLIVE2001684, TLIVE2002046, TLIVE2007607

The result of comparative analysis of cDNA libraries derived from lung tumor (TLUNG) and normal lung (HLUNG) (Table 16) showed that the genes whose expression levels differed between the two were the 47 and nine clones listed below.

BRCAN2021028, BRHIP2000819, BRSTN2016470, CTONG1000087, CTONG2028124, HCHON2006250, HEART1000074, HLUNG1000017, HLUNG2000014, HLUNG2001996, HLUNG2002465, HLUNG2002958, HLUNG2003003, HLUNG2003872, HLUNG2010464, HLUNG2011041, HLUNG2011298, HLUNG2012049, HLUNG2012287, HLUNG2012727, HLUNG2013204, HLUNG2013304, HLUNG2013622, HLUNG2013851, HLUNG2014262, HLUNG2014288, HLUNG2014449, HLUNG2015617, HLUNG2017350, HLUNG2017546, HLUNG2017806, HLUNG2019058, HSYRA2008376, KIDNE2012945, NT2RI2003993, NT2RP7013795, OCBBF3000483, SPLEN2028914, SPLEN2031547, SYNOV4007671, TESOP1000127, TESTI2003573, TESTI4000014, TESTI4037156, TRACH2005811, TRACH3004068, UTERU2005621

FEBRA2028516, HCHON2000508, HLUNG2013350, HLUNG2015418, HLUNG2015548, HLUNG2016862, NT2RI2009583, TESTI2015626, TRACH2019672

The result of comparative analysis of cDNA libraries derived from ovary tumor (TOVER) and normal ovary (NOVER) (Table 17A) showed the genes whose expression levels differed between the two were the 16 clones listed below.

CTONG2019788, FEBRA2014213, HLUNG2017546, NOVAR2000136, NOVAR2000710, NOVAR2000962, NOVAR2001108, NOVAR2001783, OCBBF3007516, TESTI2052693, TOVAR2000649, TOVAR2001281, TOVAR2001730, TOVAR2002247, TOVAR2002549, TRACH3004068

The result of comparative analysis of cDNA libraries derived from ovary tumor (TOVER) and normal ovary (NOVER) (Table 17B) showed the gene whose expression level was different between the two was the one clone described below. There was no difference in expression levels of this gene between normal and diseased ovaries, however there was a significant difference in the expression level in both tumorous and normal ovaries when compared with in other tissues. Thus, this gene is an ovary-specific gene and can be used as a diagnostic marker due to its association with disease.

TESTI2015626

The result of comparative analysis of cDNA libraries derived from stomach tumor (TSTOM) and normal stomach (STOMA) (Table 18) showed that the genes whose expression levels differed between the two were the 31 and five clones listed below.

BRACE2024627, BRAWH2014645, BRCAN2028355, BRHIP2000819, BRSTN2016470, BRTHA3003490, COLON2002443, HEART1000010, HLUNG2002465, KIDNE2001847, NT2RP7000466, PUAEN2006328, SMINT2001818, STOMA1000189, STOMA2003444, STOMA2004294, STOMA2004925, STOMA2008546, SYNOV4007671, TESTI4000014, TESTI4010851, THYMU2035735, TRACH2001549, TRACH2005811, TRACH2025535, TSTOM1000135, TSTOM2000442, TSTOM2000553, TSTOM2002672, UTERU2006115, UTERU3001572

FEBRA2008692, NT2RI2009583, STOMA2003158, STOMA2004893, TESTI2015626

The result of comparative analysis of cDNA libraries derived from uterine tumor (TUTER) and normal uterus (UTERU) (Table 19) showed that the genes whose expression levels differed between the two were the 244 and 34 clones listed below.

BNGH42007788, BRACE1000186, BRACE2030341, BRACE3008772, BRACE3009747, BRACE3010428, BRACE3027478, BRALZ2017359, BRAWH2014645, BRAWH3000314, BRAWH3001326, BRAWH3002574, BRAWH3002821, BRAWH3003727, BRAWH3007592, BRCAN2009432, BRCAN2028355, BRHIP3007586, BRHIP3008344, BRHIP3008565, BRSSN2006892, BRSTN2001067, BRSTN2016470, BRTHA2010608, BRTHA3003074, CTONG1000087, CTONG1000467, CTONG2028124, CTONG3001123, CTONG3008894, CTONG3009028, CTONG3009239, FCBBF3004847, FEBRA2026984, FEBRA2028618, HCHON2000244, HCHON2000418, HCHON2000626, HCHON2001084, HCHON2001217, HCHON2005921, HCHON2006250, HCHON2008444, HLUNG2003003, HSYRA2008376, KIDNE2002252, MESAN2014295, NOVAR2000710, NT2RI2008724, NT2RI2014247, NT2RI2014733, NT2RI3002892, NT2RI3005724, NT2RI3006284, NT2RI3006340, NT2RI3006673, NT2RI3007291, NT2RI3007543, NT2RP7004123, NT2RP7005529, NT2RP7009147, NT2RP7017474, OCBBF2007028, OCBBF2020741, OCBBF2024850, OCBBF2036743, OCBBF3000483, PLACE6001185, PLACE7000514, PUAEN2007044, PUAEN2009655, SKNSH2000482, SPLEN2006122, SPLEN2016554, SPLEN2031547, SPLEN2036932, STOMA1000189, STOMA2004925, SYNOV2017055, SYNOV4001395, SYNOV4002346, SYNOV4008440, TCERX2000613, TESOP2002273, TESTI4000014, TESTI4008797, TESTI4009286, TESTI4012702, TESTI4013675, TESTI4014159, TESTI4018886, TESTI4029671, TESTI4037156, THYMU2008725, THYMU2031890, THYMU2033070, THYMU2035735, THYMU3001472, TRACH1000205, TRACH2001443, TRACH2001549, TRACH2005811, TRACH2007834, TRACH2008300, TRACH3002192, TRACH3003379, TRACH3004068, TRACH3004721, TRACH3007479, TUTER1000122, TUTER2000425, TUTER2000904, TUTER2000916, TUTER2001387, TUTER2002729, UTERU1000024, UTERU1000031, UTERU1000148, UTERU1000249, UTERU1000337, UTERU1000339, UTERU2000649, UTERU2001409, UTERU2002410, UTERU2002841, UTERU2004688, UTERU2004929, UTERU2005004, UTERU2005621, UTERU2006115, UTERU2006137, UTERU2006568, UTERU2007444, UTERU2007520, UTERU2007724, UTERU2008347, UTERU2014678, UTERU2017762, UTERU2019491, UTERU2019681, UTERU2019706, UTERU2019940, UTERU2020491, UTERU2020718, UTERU2021163, UTERU2021380, UTERU2022020, UTERU2022981, UTERU2023039, UTERU2023175, UTERU2023651, UTERU2023712, UTERU2024002, UTERU2024656, UTERU2025025, UTERU2025645, UTERU2025891, UTERU2026025, UTERU2026090, UTERU2026203, UTERU2027591, UTERU2029953, UTERU2030213, UTERU2030280, UTERU2031084, UTERU2031268, UTERU2031521, UTERU2031703, UTERU2031851, UTERU2033375, UTERU2033382, UTERU2035114, UTERU2035323, UTERU2035328, UTERU2035331, UTERU2035452, UTERU2035469, UTERU2035503, UTERU2035745, UTERU2036089, UTERU2037361, UTERU2037577, UTERU2038251, UTERU3000226, UTERU3000645, UTERU3000665, UTERU3000828, UTERU3000899, UTERU3001059, UTERU3001240, UTERU3001542, UTERU3001571, UTERU3001572, UTERU3001585, UTERU3001652, UTERU3001766, UTERU3001988, UTERU3002209, UTERU3002218, UTERU3002383, UTERU3002667, UTERU3002731, UTERU3002768, UTERU3002786, UTERU3002993, UTERU3003116, UTERU3003135, UTERU3003178, UTERU3003465, UTERU3003523, UTERU3003776, UTERU3004523, UTERU3004616, UTERU3004709, UTERU3004992, UTERU3005049, UTERU3005205, UTERU3005230, UTERU3005460, UTERU3005585, UTERU3005907, UTERU3005970, UTERU3006008, UTERU3006308, UTERU3007134, UTERU3007419, UTERU3007640, UTERU3007913, UTERU3008660, UTERU3008671, UTERU3009259, UTERU3009490, UTERU3009517, UTERU3009690, UTERU3009871, UTERU3009979, UTERU3011063, UTERU3015086, UTERU3015500, UTERU3016789, UTERU3018081, UTERU3018154, UTERU3018616, UTERU3018711

ADRGL2000042, BRHIP3000017, CTONG2003348, CTONG2019822, CTONG2020378, CTONG2020411, CTONG2024031, FEBRA2028516, HCASM2008536, HCHON2000743, IMR322001879, MESAN2005303, NT2RI2009583, OCBBF2008144, PERIC2007068, SPLEN2039379, TESTI2015626, TESTI4013894, TUTER2000057, UTERU2004299, UTERU2008040, UTERU2011220, UTERU2019534, UTERU2021820, UTERU2028734, UTERU2032279, UTERU2033577, UTERU2035978, UTERU3000402, UTERU3000738, UTERU3001053, UTERU3014791, UTERU3015412, UTERU3017176

The result of comparative analysis of cDNA libraries derived from tongue cancer (CTONG) and normal tongue (NTONG) (Table 20) showed that the genes whose expression levels differed between the two were the 166 and 31 clones listed below.

BNGH42007788, BRACE1000186, BRACE2006319, BRACE3010428, BRACE3012364, BRAMY2020058, BRAMY3002803, BRAWH2001671, BRAWH2014645, BRAWH3002574, BRCAN2009432, BRCAN2015371, BRCAN2020710, BRHIP2004814, BRHIP3018797, BRTHA2003461, BRTHA3003490, CTONG1000087, CTONG1000088, CTONG1000288, CTONG1000302, CTONG1000341, CTONG1000467, CTONG1000488, CTONG1000508, CTONG1000540, CTONG2000042, CTONG2001877, CTONG2004062, CTONG2006798, CTONG2008233, CTONG2009423, CTONG2009531, CTONG2010803, CTONG2013178, CTONG2017500, CTONG2019248, CTONG2019652, CTONG2019704, CTONG2019788, CTONG2019833, CTONG2020127, CTONG2020522, CTONG2020638, CTONG2020806, CTONG2021132, CTONG2022153, CTONG2022601, CTONG2023021, CTONG2023512, CTONG2024206, CTONG2024749, CTONG2025496, CTONG2025516, CTONG2025900, CTONG2026920, CTONG2027327, CTONG2028124, CTONG2028687, CTONG3000084, CTONG3000657, CTONG3000686, CTONG3000707, CTONG3000896, CTONG3001123, CTONG3001370, CTONG3001420, CTONG3001560, CTONG3002020, CTONG3002127, CTONG3002412, CTONG3002674, CTONG3003179, CTONG3003483, CTONG3003652, CTONG3003654, CTONG3003737, CTONG3003905, CTONG3003972, CTONG3004072, CTONG3004712, CTONG3005325, CTONG3005648, CTONG3005713, CTONG3005813, CTONG3006067, CTONG3006186, CTONG3006650, CTONG3007444, CTONG3007528, CTONG3007586, CTONG3007870, CTONG3008252, CTONG3008258, CTONG3008496, CTONG3008566, CTONG3008639, CTONG3008831, CTONG3008894, CTONG3008951, CTONG3009028, CTONG3009227, CTONG3009239, CTONG3009328, CTONG3009385, FEBRA2007544, FEBRA2007801, FEBRA2021966, FEBRA2025427, HCHON2000028, HCHON2001217, HHDPC1000118, HSYRA2008376, KIDNE2001847, KIDNE2002252, MESAN2006563, NT2RI2008724, NT2RI2018883, NT2RI3000622, NT2RI3006284, NT2RI3006673, NT2RI3007543, NT2RI3007757, NT2RP7004123, NT2RP7009147, NT2RP7014005, NTONG2000413, NTONG2003852, NTONG2005277, NTONG2005969, NTONG2006354, NTONG2007249, NTONG2007517, NTONG2008088, NTONG2008672, OCBBF2001794, OCBBF2006151, PEBLM2004666, PEBLM2005183, SPLEN2002467, SPLEN2029912, SPLEN2031547, SYNOV4007671, SYNOV4008440, TBAES2002197, TESOP2002273, TESTI2009474, TESTI4000014, TESTI4000209, TESTI4008018, TESTI4009286, TESTI4010851, TESTI4012702, TESTI4013675, THYMU2031847, THYMU2033308, TLIVE2002690, TRACH2005811, TRACH2007059, TRACH2025535, TRACH3001427, TSTOM2000553, UTERU2005621, UTERU2017762, UTERU2023175, UTERU3001572

BLADE2006830, BRHIP3000017, CTONG1000113, CTONG2003348, CTONG2004000, CTONG2008721, CTONG2015596, CTONG2015633, CTONG2016942, CTONG2019822, CTONG2020374, CTONG2020378, CTONG2020411, CTONG2020974, CTONG2024031, CTONG2028758, CTONG3001501, CTONG3002552, CTONG3003598, CTONG3004550, CTONG3004726, CTONG3009287, FEBRA2008692, FEBRA2028516, HCHON2000508, NT2RI2009583, NTONG2008093, PERIC2007068, TESOP2007384, TLIVE2002046, TRACH2000862

These genes are involved in cancer.

Expression frequency analysis is used when searching for genes involved in development and differentiation, where the expression levels of genes in developing and/or differentiating tissues and/or cells are compared with those in adult tissues and/or cells. Genes involved in tissue development and/or differentiation are genes which participate in tissue construction and function expression. These genes are thus useful genes available for medicine aiming at regeneration of injured tissues.

By using information on gene expression frequency gained from the database of the nucleotide sequences of the 1,402,069 clones as described above, genes whose expression frequencies differed between developing and/or differentiating tissues and/or cells, and adult tissues and/or cells, were analyzed.

The result of comparative analysis of cDNA libraries derived from fetal brain (FCBBF, FEBRA or OCBBF) and adult brain (BRACE, BRALZ, BRAMY, BRAWH, BRCAN, BRCOC, BRHIP, BRSSN, BRSTN or BRTHA) (Table 21) showed that the genes whose expression levels differed between the two were the 1,035 and 139 clones listed below.

ADRGL2009146, ADRGL2012038, ADRGL2012179, ASTRO1000009, ASTRO2003960, ASTRO3000482, BLADE1000176, BLADE2001371, BLADE2004089, BLADE2008398, BNGH42007788, BRACE1000186, BRACE1000258, BRACE1000533, BRACE1000572, BRACE2003639, BRACE2005457, BRACE2006319, BRACE2008594, BRACE2010489, BRACE2011747, BRACE2014306, BRACE2014475, BRACE2014657, BRACE2015058, BRACE2015314, BRACE2016981, BRACE2018762, BRACE2024627, BRACE2026836, BRACE2027258, BRACE2027970, BRACE2028970, BRACE2029112, BRACE2029849, BRACE2030326, BRACE2030341, BRACE2030884, BRACE2031154, BRACE2031389, BRACE2031527, BRACE2031531, BRACE2031899, BRACE2032044, BRACE2032329, BRACE2032385, BRACE2032538, BRACE2032823, BRACE2033720, BRACE2035381, BRACE2035441, BRACE2036005, BRACE2036096, BRACE2036830, BRACE2036834, BRACE2037847, BRACE2038114, BRACE2038329, BRACE2038551, BRACE2039249, BRACE2039327, BRACE2039475, BRACE2039734, BRACE2040138, BRACE2040325, BRACE2041009, BRACE2041200, BRACE2041264, BRACE2042550, BRACE2043142, BRACE2043248, BRACE2043349, BRACE2043665, BRACE2044286, BRACE2044816, BRACE2044949, BRACE2045300, BRACE2045428, BRACE2045596, BRACE2045772, BRACE2045947, BRACE2045954, BRACE2046251, BRACE2046295, BRACE2047011, BRACE2047350, BRACE2047377, BRACE2047385, BRACE3000071, BRACE3000697, BRACE3000787, BRACE3000840, BRACE3000973, BRACE3001002, BRACE3001217, BRACE3001391, BRACE3001595, BRACE3001754, BRACE3002298, BRACE3002390, BRACE3002508, BRACE3003004, BRACE3003192, BRACE3003595, BRACE3003698, BRACE3004058, BRACE3004113, BRACE3004150, BRACE3004358, BRACE3004435, BRACE3004772, BRACE3004783, BRACE3004843, BRACE3004880, BRACE3005145, BRACE3005225, BRACE3005430, BRACE3005499, BRACE3006185, BRACE3006226, BRACE3006462, BRACE3006872, BRACE3007322, BRACE3007472, BRACE3007480, BRACE3007559, BRACE3007625, BRACE3007642, BRACE3007767, BRACE3008036, BRACE3008092, BRACE3008137, BRACE3008384, BRACE3008720, BRACE3008772, BRACE3009090, BRACE3009237, BRACE3009297, BRACE3009377, BRACE3009574, BRACE3009701, BRACE3009708, BRACE3009724, BRACE3009747, BRACE3010397, BRACE3010428, BRACE3011271, BRACE3011421, BRACE3011505, BRACE3012364, BRACE3012930, BRACE3013119, BRACE3013576, BRACE3013740, BRACE3013780, BRACE3014005, BRACE3014068, BRACE3014231, BRACE3014317, BRACE3014807, BRACE3015027, BRACE3015121, BRACE3015262, BRACE3015521, BRACE3015894, BRACE3016884, BRACE3018308, BRACE3018963, BRACE3019055, BRACE3019084, BRACE3020194, BRACE3020286, BRACE3020594, BRACE3022769, BRACE3023912, BRACE3024073, BRACE3024659, BRACE3024662, BRACE3025153, BRACE3025457, BRACE3025531, BRACE3025630, BRACE3026008, BRACE3026075, BRACE3026735, BRACE3027242, BRACE3027326, BRACE3027478, BRACE3030103, BRACE3031838, BRACE3032983, BRACE3040856, BRACE3045033, BRALZ2011796, BRALZ2012183, BRALZ2012848, BRALZ2014484, BRALZ2016085, BRALZ2016498, BRALZ2017359, BRAMY2001473, BRAMY2003008, BRAMY2004771, BRAMY2005052, BRAMY2017528, BRAMY2019300, BRAMY2019963, BRAMY2019985, BRAMY2020058, BRAMY2020270, BRAMY2021498, BRAMY2028856, BRAMY2028914, BRAMY2029602, BRAMY2030098, BRAMY2030109, BRAMY2030702, BRAMY2030703, BRAMY2030799, BRAMY2031317, BRAMY2031377, BRAMY2031442, BRAMY2032014, BRAMY2032242, BRAMY2032317, BRAMY2033003, BRAMY2033116, BRAMY2033267, BRAMY2033594, BRAMY2034185, BRAMY2034920, BRAMY2034993, BRAMY2036387, BRAMY2036396, BRAMY2036567, BRAMY2036699, BRAMY2036913, BRAMY2037823, BRAMY2038100, BRAMY2038484, BRAMY2038846, BRAMY2038904, BRAMY2039872, BRAMY2040478, BRAMY2040592, BRAMY2041261, BRAMY2041378, BRAMY2041542, BRAMY2042612, BRAMY2042641, BRAMY2042760, BRAMY2042918, BRAMY2044078, BRAMY2044246, BRAMY2045036, BRAMY2046478, BRAMY2046742, BRAMY2046989, BRAMY2047169, BRAMY2047420, BRAMY2047676, BRAMY2047746, BRAMY2047751, BRAMY2047765, BRAMY2047884, BRAMY3000206, BRAMY3000213, BRAMY3001401, BRAMY3001794, BRAMY3002312, BRAMY3002620, BRAMY3002803, BRAMY3002805, BRAMY3004224, BRAMY3004672, BRAMY3004900, BRAMY3004919, BRAMY3005091, BRAMY3005932, BRAMY3006297, BRAMY3007206, BRAMY3007609, BRAMY3008466, BRAMY3008505, BRAMY3008650, BRAMY3009811, BRAMY3010411, BRAMY4000095, BRAMY4000229, BRAMY4000277, BRASW1000125, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2002562, BRCAN2002856, BRCAN2002944, BRCAN2002948, BRCAN2003703, BRCAN2003746, BRCAN2003987, BRCAN2004355, BRCAN2005436, BRCAN2006063, BRCAN2006290, BRCAN2006297, BRCAN2006450, BRCAN2007144, BRCAN2007409, BRCAN2007426, BRCAN2008528, BRCAN2009203, BRCAN2009432, BRCAN2010376, BRCAN2011254, BRCAN2011602, BRCAN2012355, BRCAN2012481, BRCAN2013655, BRCAN2013660, BRCAN2014143, BRCAN2014602, BRCAN2014881, BRCAN2015371, BRCAN2015464, BRCAN2016433, BRCAN2016619, BRCAN2017442, BRCAN2017717, BRCAN2017905, BRCAN2018935, BRCAN2019387, BRCAN2020710, BRCAN2021028, BRCAN2024451, BRCAN2024563, BRCAN2025712, BRCAN2028355, BRCOC2000670, BRCOC2003213, BRCOC2007034, BRCOC2014033, BRCOC2016525, BRCOC2019934, BRCOC2020142, BRHIP2000691, BRHIP2000819, BRHIP2000826, BRHIP2000920, BRHIP2001074, BRHIP2001805, BRHIP2001927, BRHIP2002122, BRHIP2002172, BRHIP2002346, BRHIP2003242, BRHIP2003786, BRHIP2003917, BRHIP2004312, BRHIP2004359, BRHIP2004814, BRHIP2004883, BRHIP2005236, BRHIP2005354, BRHIP2005600, BRHIP2005719, BRHIP2005752, BRHIP2005932, BRHIP2006800, BRHIP2007616, BRHIP2007741, BRHIP2009340, BRHIP2009414, BRHIP2009474, BRHIP2013699, BRHIP2014228, BRHIP2021615, BRHIP2022221, BRHIP2024146, BRHIP2024165, BRHIP2026061, BRHIP2026288, BRHIP2029176, BRHIP2029393, BRHIP3000339, BRHIP3000526, BRHIP3001283, BRHIP3006683, BRHIP3007483, BRHIP3007586, BRHIP3008183, BRHIP3008313, BRHIP3008344, BRHIP3008405, BRHIP3008565, BRHIP3008598, BRHIP3008997, BRHIP3009099, BRHIP3009448, BRHIP3011241, BRHIP3013765, BRHIP3013897, BRHIP3015751, BRHIP3016213, BRHIP3018797, BRHIP3020182, BRHIP3024118, BRHIP3024533, BRHIP3024725, BRHIP3025161, BRHIP3025702, BRHIP3026097, BRHIP3027137, BRHIP3027854, BRSSN2000684, BRSSN2003086, BRSSN2004496, BRSSN2004719, BRSSN2006892, BRSSN2008549, BRSSN2008797, BRSSN2011262, BRSSN2011738, BRSSN2013874, BRSSN2014299, BRSSN2014424, BRSSN2014556, BRSSN2018581, BRSSN2018925, BRSTN2000872, BRSTN2001067, BRSTN2001613, BRSTN2002400, BRSTN2003835, BRSTN2004863, BRSTN2004987, BRSTN2005721, BRSTN2006865, BRSTN2007000, BRSTN2007284, BRSTN2008052, BRSTN2008283, BRSTN2008418, BRSTN2008457, BRSTN2010363, BRSTN2010500, BRSTN2010750, BRSTN2012320, BRSTN2012380, BRSTN2015015, BRSTN2016470, BRSTN2016678, BRSTN2017237, BRSTN2017771, BRSTN2018083, BRSTN2019129, BRTHA1000311, BRTHA2000855, BRTHA2001462, BRTHA2002115, BRTHA2002281, BRTHA2002376, BRTHA2002442, BRTHA2002493, BRTHA2002608, BRTHA2002808, BRTHA2003030, BRTHA2003110, BRTHA2003116, BRTHA2003461, BRTHA2004821, BRTHA2004978, BRTHA2005579, BRTHA2005956, BRTHA2006075, BRTHA2006146, BRTHA2006194, BRTHA2007122, BRTHA2007422, BRTHA2007603, BRTHA2008316, BRTHA2008335, BRTHA2008527, BRTHA2008535, BRTHA2008955, BRTHA2009311, BRTHA2009846, BRTHA2009972, BRTHA2010073, BRTHA2010608, BRTHA2010884, BRTHA2010907, BRTHA2011194, BRTHA2011351, BRTHA2011500, BRTHA2011641, BRTHA2012392, BRTHA2012562, BRTHA2012980, BRTHA2013262, BRTHA2013460, BRTHA2013707, BRTHA2014792, BRTHA2014828, BRTHA2015406, BRTHA2015478, BRTHA2015696, BRTHA2015878, BRTHA2016215, BRTHA2016496, BRTHA2016543, BRTHA2017353, BRTHA2017985, BRTHA2018165, BRTHA2018344, BRTHA2018591, BRTHA2018624, BRTHA2018707, BRTHA2019014, BRTHA2019022, BRTHA2019048, BRTHA3000273, BRTHA3000297, BRTHA3000633, BRTHA3001721, BRTHA3002401, BRTHA3002427, BRTHA3002933, BRTHA3003074, BRTHA3003343, BRTHA3003449, BRTHA3003474, BRTHA3003490, BRTHA3004475, BRTHA3005046, BRTHA3006856, BRTHA3007113, BRTHA3007148, BRTHA3007319, BRTHA3007769, BRTHA3008143, BRTHA3008310, BRTHA3008386, BRTHA3008520, BRTHA3008778, BRTHA3009037, BRTHA3009090, BRTHA3009291, BRTHA3010366, BRTHA3013884, BRTHA3015815, BRTHA3015910, BRTHA3016845, BRTHA3016917, BRTHA3017047, BRTHA3017589, BRTHA3017848, BRTHA3018514, BRTHA3018617, BRTHA3018656, BRTHA3019105, COLON2001721, CTONG1000087, CTONG1000088, CTONG1000467, CTONG2000042, CTONG2008233, CTONG2009423, CTONG2017500, CTONG2019248, CTONG2019788, CTONG2020522, CTONG2023021, CTONG2028124, CTONG3000657, CTONG3001123, CTONG3001370, CTONG3002412, CTONG3004072, CTONG3005813, CTONG3008894, CTONG3009028, CTONG3009239, CTONG3009328, DFNES2000146, DFNES2011239, DFNES2011499, FCBBF1000297, FCBBF2001183, FCBBF3001977, FCBBF3002163, FCBBF3003435, FCBBF3004502, FCBBF3004847, FCBBF3006171, FCBBF3007242, FCBBF3007540, FCBBF3008944, FCBBF3009888, FCBBF3012170, FCBBF3012288, FCBBF3013307, FCBBF3013846, FCBBF3021576, FCBBF3021940, FCBBF3023443, FCBBF3023895, FCBBF3025730, FCBBF3027717, FCBBF4000076, FEBRA1000030, FEBRA2000253, FEBRA2006396, FEBRA2007544, FEBRA2007708, FEBRA2007793, FEBRA2007801, FEBRA2008287, FEBRA2008311, FEBRA2008360, FEBRA2008468, FEBRA2010719, FEBRA2014213, FEBRA2015588, FEBRA2020484, FEBRA2020582, FEBRA2020668, FEBRA2020886, FEBRA2021339, FEBRA2021571, FEBRA2021908, FEBRA2021966, FEBRA2024136, FEBRA2024150, FEBRA2024343, FEBRA2024744, FEBRA2025427, FEBRA2026984, FEBRA2027082, FEBRA2027297, FEBRA2027352, FEBRA2028366, FEBRA2028477, FEBRA2028618, HCASM2007047, HCHON2000028, HCHON2000212, HCHON2000244, HCHON2000626, HCHON2001084, HCHON2001217, HCHON2002676, HCHON2005921, HCHON2006250, HCHON2007881, HCHON2008112, HEART1000074, HEART2007031, HHDPC1000118, HLUNG2001996, HLUNG2002465, HLUNG2003003, HSYRA2009075, IMR322000127, IMR322000917, IMR322001380, IMR322002035, KIDNE2000665, KIDNE2002252, KIDNE2005543, KIDNE2006580, KIDNE2011314, MESAN2006563, MESAN2012054, MESAN2015515, MESTC1000042, NB9N41000340, NESOP2001752, NHNPC2001223, NOVAR2001783, NT2NE2005890, NT2NE2006909, NT2NE2008060, NT2RI2003993, NT2RI2005166, NT2RI2008724, NT2RI2012659, NT2RI2014733, NT2RI2018311, NT2RI2019751, NT2RI3000622, NT2RI3001515, NT2RI3002842, NT2RI3002892, NT2RI3003382, NT2RI3004510, NT2RI3005403, NT2RI3005724, NT2RI3006284, NT2RI3006673, NT2RI3007291, NT2RI3007543, NT2RI3008055, NT2RP7004123, NT2RP7005529, NT2RP7009030, NT2RP7009147, NT2RP7010599, NT2RP7014005, NT2RP7015512, NT2RP7017474, NTONG2000413, NTONG2005969, NTONG2008088, OCBBF1000254, OCBBF2001794, OCBBF2002124, OCBBF2003819, OCBBF2004826, OCBBF2004883, OCBBF2005428, OCBBF2006005, OCBBF2006058, OCBBF2006151, OCBBF2006567, OCBBF2006764, OCBBF2007028, OCBBF2007068, OCBBF2007114, OCBBF2007428, OCBBF2007478, OCBBF2007610, OCBBF2008770, OCBBF2009788, OCBBF2009926, OCBBF2010140, OCBBF2010416, OCBBF2017516, OCBBF2019327, OCBBF2019823, OCBBF2020343, OCBBF2020453, OCBBF2020639, OCBBF2020741, OCBBF2020801, OCBBF2020838, OCBBF2021020, OCBBF2021286, OCBBF2021323, OCBBF2021788, OCBBF2022351, OCBBF2022574, OCBBF2023162, OCBBF2023643, OCBBF2024719, OCBBF2024781, OCBBF2024850, OCBBF2025028, OCBBF2025458, OCBBF2025527, OCBBF2025730, OCBBF2026645, OCBBF2027423, OCBBF2027478, OCBBF2028173, OCBBF2028935, OCBBF2029901, OCBBF2030354, OCBBF2030517, OCBBF2030574, OCBBF2030708, OCBBF2031167, OCBBF2031366, OCBBF2032590, OCBBF2032599, OCBBF2032611, OCBBF2032671, OCBBF2033869, OCBBF2035110, OCBBF2035214, OCBBF2035564, OCBBF2035885, OCBBF2035916, OCBBF2036476, OCBBF2036743, OCBBF2037068, OCBBF2037340, OCBBF2037398, OCBBF2037547, OCBBF2037598, OCBBF2037638, OCBBF2038317, OCBBF3000296, OCBBF3000483, OCBBF3002553, OCBBF3002600, OCBBF3003320, OCBBF3003592, OCBBF3004314, OCBBF3006802, OCBBF3007516, OCBBF3008230, OCBBF3009279, PEBLM2004666, PERIC2000889, PERIC2002766, PERIC2003720, PLACE6001185, PLACE6019385, PUAEN2002489, PUAEN2005930, PUAEN2006701, PUAEN2007044, PUAEN2009174, PUAEN2009655, RECTM2001347, SKMUS2000757, SKNMC2002402, SKNSH2000482, SMINT2001818, SPLEN2001599, SPLEN2002467, SPLEN2006122, SPLEN2010912, SPLEN2012624, SPLEN2025491, SPLEN2027268, SPLEN2028914, SPLEN2029912, SPLEN2031424, SPLEN2031547, SPLEN2032154, SPLEN2034781, SPLEN2036821, SPLEN2036932, SPLEN2037194, SPLEN2038345, SPLEN2042303, SYNOV1000374, SYNOV2005216, SYNOV2014400, SYNOV4002346, SYNOV4002883, SYNOV4007430, SYNOV4007671, SYNOV4008440, TESOP2001605, TESOP2002273, TESOP2002451, TESOP2002950, TESTI1000330, TESTI2003573, TESTI2009474, TESTI2049246, TESTI4000014, TESTI4000209, TESTI4000349, TESTI4001100, TESTI4001561, TESTI4002290, TESTI4002647, TESTI4005857, TESTI4006137, TESTI4006326, TESTI4008797, TESTI4009286, TESTI4010377, TESTI4010851, TESTI4010928, TESTI4011161, TESTI4012702, TESTI4013675, TESTI4013817, TESTI4014159, TESTI4014175, TESTI4014306, TESTI4014694, TESTI4014818, TESTI4019843, TESTI4021478, TESTI4022936, TESTI4024420, TESTI4027821, TESTI4029836, TESTI4037156, TESTI4037188, TESTI4046819, THYMU2001090, THYMU2011736, THYMU2014353, THYMU2016204, THYMU2016523, THYMU2023967, THYMU2025707, THYMU2030264, THYMU2031341, THYMU2031890, THYMU2032696, THYMU2032825, THYMU2033308, THYMU2033787, THYMU2034374, THYMU2035735, THYMU2037226, THYMU2039315, THYMU2039780, THYMU2040975, THYMU3001083, THYMU3001234, THYMU3001379, THYMU3003309, THYMU3004835, THYMU3006485, THYMU3007137, THYMU3008171, TKIDN2009641, TKIDN2009889, TKIDN2010934, TKIDN2013287, TKIDN2015788, TLIVE2001327, TLIVE2004320, TRACH1000205, TRACH2001443, TRACH2001549, TRACH2001684, TRACH2005811, TRACH2006049, TRACH2007834, TRACH2008300, TRACH2023299, TRACH2025344, TRACH2025535, TRACH2025911, TRACH3000014, TRACH3001427, TRACH3002192, TRACH3004068, TRACH3004721, TRACH3005294, TRACH3006038, TRACH3006412, TRACH3007479, TRACH3008093, TRACH3009455, TSTOM1000135, TUTER1000122, TUTER2000904, UTERU2002410, UTERU2004929, UTERU2005621, UTERU2006115, UTERU2007520, UTERU2014678, UTERU2019706, UTERU2019940, UTERU2021163, UTERU2023039, UTERU2023175, UTERU2026203, UTERU2030213, UTERU2030280, UTERU3000226, UTERU3000899, UTERU3001571, UTERU3001572, UTERU3001766, UTERU3003135, UTERU3004709, UTERU3005230, UTERU3005460, UTERU3005907, UTERU3005970, UTERU3006308, UTERU3007419, UTERU3007640, UTERU3007913, UTERU3009259, UTERU3009517, UTERU3009871

ADRGL2000042, BLADE2006830, BRACE2002589, BRACE2003609, BRACE2009318, BRACE2011677, BRACE2029396, BRACE2037299, BRACE2039823, BRACE2039832, BRACE2043105, BRACE3001058, BRACE3001113, BRACE3003026, BRACE3003053, BRACE3009127, BRACE3010076, BRACE3015829, BRACE3021148, BRALZ2017844, BRAMY2019111, BRAMY2035070, BRAMY2035449, BRAMY2035718, BRAMY2038516, BRAMY2039341, BRAMY2040159, BRAMY2041434, BRAMY2045471, BRAMY3004800, BRAWH1000369, BRAWH2006207, BRAWH2006395, BRAWH2008993, BRAWH2009393, BRAWH2010552, BRAWH3007441, BRAWH3009017, BRCAN2002473, BRCAN2002854, BRCAN2003070, BRCAN2014229, BRCOC2019841, BRHIP2002722, BRHIP2003272, BRHIP2005271, BRHIP2005724, BRHIP2006617, BRHIP2008389, BRHIP2012360, BRHIP2017553, BRHIP2026877, BRHIP3000017, BRHIP3000240, BRHIP3008314, BRHIP3026052, BRSTN2013354, BRTHA2002133, BRTHA2002702, BRTHA2007060, BRTHA2010033, BRTHA2011321, BRTHA2013426, BRTHA2013610, BRTHA2016318, BRTHA2017364, BRTHA2017972, BRTHA2018011, BRTHA2018443, BRTHA3000296, BRTHA3003000, BRTHA3008826, CTONG2008721, CTONG2020374, CTONG2020378, CTONG2020411, CTONG2024031, CTONG3004726, FCBBF1000509, FCBBF3010361, FCBBF3027854, FEBRA2000790, FEBRA2001990, FEBRA2006519, FEBRA2008692, FEBRA2014122, FEBRA2027609, FEBRA2028516, HCASM2003018, HCHON2000508, HCHON2000743, HCHON2004858, HSYRA2005628, IMR322001879, NT2RI2009583, NT2RP8000521, OCBBF2003327, OCBBF2005433, OCBBF2006987, OCBBF2008144, OCBBF2009583, OCBBF2011669, OCBBF2019684, OCBBF2020048, OCBBF2030116, OCBBF2032274, OCBBF2034637, OCBBF3002654, OCBBF3003761, OCBBF3004972, PERIC2007068, PUAEN2006335, SPLEN2016932, SPLEN2039379, SYNOV2006620, SYNOV2021953, TESTI1000266, TESTI2015626, TESTI2026647, TESTI4000214, TESTI4001984, TESTI4008058, TESTI4013894, TESTI4015442, TESTI4017714, TESTI4025268, TESTI4025547, TESTI4026207, TESTI4032090, THYMU2004284, THYMU2040925, THYMU3000360, TKIDN2018926, TLIVE2002046, TRACH3000134, UTERU2008040, UTERU2011220, UTERU2021820, UTERU2028734

The result of comparative analysis of cDNA libraries derived from fetal heart (FEHRT) and adult heart (HEART) (Table 22) showed that the genes whose expression levels differed between the two were the 34 and two clones listed below.

BRAMY2040592, BRAWH2001671, BRSTN2016470, CTONG2017500, CTONG2028124, CTONG3000657, D3OST3000169, FEBRA2008287, HCHON2000244, HCHON2000626, HEART1000010, HEART1000074, HEART1000088, HEART1000139, HEART2001680, HEART2001756, HEART2006131, HEART2006909, HEART2007031, HEART2010391, HEART2010492, HEART2010495, KIDNE2000665, NB9N41000340, NT2RI2003993, NT2RI3002892, OCBBF2024850, SKMUS2006394, SMINT2001818, TESTI4000209, TKIDN2015788, TRACH3002192, TRACH3005294, TRACH3007479 HEART2009680, THYMU2004284

The result of comparative analysis of cDNA libraries derived from fetal kidney (FEKID) and adult kidney (KIDNE) (Table 23) showed that the genes whose expression levels differed between the two were the 40 and two clones listed below.

BRACE2043665, BRACE3010428, BRSTN2016470, CTONG1000087, CTONG2028124, CTONG3008894, HCASM2003415, HCHON2000244, HEART1000074, HHDPC1000118, KIDNE1000064, KIDNE2000665, KIDNE2000722, KIDNE2000832, KIDNE2000846, KIDNE2001361, KIDNE2001847, KIDNE2002252, KIDNE2002991, KIDNE2003837, KIDNE2005543, KIDNE2006580, KIDNE2010264, KIDNE2011314, KIDNE2011532, KIDNE2011635, KIDNE2012945, KIDNE2013095, PEBLM2004666, PLACE6019385, STOMA1000189, SYNOV4007671, TBAES2001258, TESOP2002451, TESTI4000014, TESTI4012702, THYMU2032014, TRACH2001684, TRACH2007834, UTERU2023175 NT2RI2009583, OCBBF2008144

The result of comparative analysis of cDNA libraries derived from fetal lung (FELNG) and adult lung (HLUNG) (Table 24) showed that the genes whose expression levels differed between the two were the 51 and eight clones listed below.

BRAWH3007592, BRCAN2021028, BRHIP2000819, BRSTN2016470, CTONG1000087, CTONG2028124, HCASM2007047, HEART1000074, HLUNG1000017, HLUNG2000014, HLUNG2001996, HLUNG2002465, HLUNG2002958, HLUNG2003003, HLUNG2003872, HLUNG2010464, HLUNG2011041, HLUNG2011298, HLUNG2012049, HLUNG2012287, HLUNG2012727, HLUNG2013204, HLUNG2013304, HLUNG2013622, HLUNG2013851, HLUNG2014262, HLUNG2014288, HLUNG2014449, HLUNG2015617, HLUNG2017350, HLUNG2017546, HLUNG2017806, HLUNG2019058, HSYRA2008376, KIDNE2012945, NT2RI2003993, NT2RI3007543, OCBBF3000483, SMINT1000192, SPLEN2028914, SPLEN2031547, STOMA1000189, SYNOV4007671, TESOP1000127, TESTI2003573, TESTI4000014, TESTI4037156, TRACH2005811, TRACH3004068, UTERU2005621, UTERU2023175

FEBRA2028516, HCHON2000508, HLUNG2013350, HLUNG2015418, HLUNG2015548, HLUNG2016862, TESTI2015626, TRACH2019672

These genes are involved in regeneration of tissues and/or cells.

For example, if a polypeptide encoded by a cDNA of the present invention is a regulatory factor for cellular conditions such as growth and differentiation, it can be used for developing medicines as follows. The polypeptide or antibody provided by the present invention is injected into cells using microinjection, and then low molecular weight compounds and such can be screened by using, as an index, change in cellular conditions (such as growth and differentiation), or activation/inhibition of a particular gene in the cell. The screening can be performed as follows.

A polypeptide of the present invention is first expressed and purified as a recombinant. This purified polypeptide is then microinjected into cells such as various cell lines, or primary culture cells, and cellular changes such as growth and differentiation are examined. Alternatively, induction of a gene whose expression is known to be involved in the change of a particular cellular condition may be detected using mRNA or polypeptide amounts. Alternatively, the amount of an intracellular molecule (low molecular weight compounds, etc.) may be detected, where that amount is changed by the function of a gene product (polypeptide) known to influence change in a particular cellular condition. The compounds to be screened (including both low and high molecular compounds) can be added to culture media and screened using, as an index, their ability to change a cellular condition.

Cell lines introduced with a gene of the present invention can be used for screening, even without microinjection. If the product of a gene of the present invention is revealed to be involved in a particular change in cellular conditions, the change in that product can be used as an index for screening. Once a compound which can activate or inhibit the function of a polypeptide of the present invention has been developed using this screening, such a compound can be practically applied in medicines.

If a polypeptide encoded by a cDNA of the present invention is a secretory or membrane protein, or a protein involved in signal transduction, glycoproteins, transcription, or disease, it can be used in functional assays for developing medicines as described below.

In case of a membrane protein, the polypeptide is very likely to function as a receptor or ligand on the cell surface. Therefore, it is possible to reveal new ligand-receptor relationships by screening membrane proteins of the present invention, based on binding activity with known or new ligands or receptors. Screening can be performed according to known methods.

For example, a ligand against a polypeptide of the present invention can be screened in the following manner. Namely, a ligand that binds to a specific polypeptide can be screened using a method comprising the steps of: (a) contacting a test sample with a polypeptide of the present invention, or partial peptide thereof, or cells expressing such, and (b) selecting a test sample that binds to said polypeptide, said partial peptide, or said cells.

Screening using cells expressing receptor polypeptides of the present invention can also be performed, for example, as follows. Receptors capable of binding to a specific polypeptide can be screened by (a) attaching sample cells to a polypeptide of the present invention or its partial peptide, and (b) selecting cells that can bind to the said polypeptide or its partial peptide.

For example, screening can be carried out as follows: a polypeptide of the present invention is first expressed, the recombinant polypeptide is purified and labeled, a binding assay is performed using various cell lines or primary cultured cells, and cells that express the receptor are selected (Growth and differentiation factors and their receptors, Shin-Seikagaku Jikken Kouza Vol. 7 (1991) Honjyo, Arai, Taniguchi, and Muramatsu edit, p203-236, Tokyo-Kagaku-Doujin). A polypeptide of the present invention can be labeled with RI such as ¹²⁵I, and enzyme (alkaline phosphatase etc.).

Alternatively, a polypeptide of the present invention may be used without labeling and then detected by using a labeled antibody against that polypeptide. Cells expressing a receptor polypeptide of the present invention, and selected using the above screening methods, can be applied as mentioned below to screen agonists or antagonists of that receptor.

Once a ligand binding to a polypeptide of the present invention, a receptor of that polypeptide, or cells expressing that receptor have been obtained by screening as described above, a compound binding to that ligand or receptor can be screened. It is also possible to screen a compound that can inhibit both bindings (agonists or antagonists of the receptor, for example) by using this binding activity as an index.

When a polypeptide of the present invention is a receptor, the screening method comprises the steps of (a) contacting the ligand with a polypeptide of the present invention, or cells expressing that polypeptide, in the presence of a test sample, (b) detecting binding activity between that polypeptide or cells expressing that polypeptide and the ligand, and (c) selecting a compound that can reduce that binding activity when compared to activity in the absence of the test sample. Furthermore, when a polypeptide of the present invention is a ligand, the screening method comprises the steps of (a) contacting the polypeptide of the present invention with its receptor or cells expressing that receptor, in the presence of a test sample, (b) detecting binding activity between the polypeptide and its receptor, or cells expressing that receptor, and (c) selecting a compound that can reduce that binding activity compared to activity in the absence of the test sample.

Examples of test samples to screen include, but are not limited to, cell extracts, expressed gene library products, synthesized low molecular compounds, synthesized peptides, and natural compounds. A compound that is isolated by the above screening can also be used as a test sample, using binding activity with a polypeptide of the present invention as an index.

A compound isolated using this screening may be a candidate agonist or antagonist of a polypeptide or polypeptide receptor of the present invention. By monitoring changes in intracellular signals, such as phosphorylation due to reduced binding between a polypeptide and its receptor, it is possible to identify whether the obtained compound is an agonist or antagonist of the polypeptide receptor of the present invention. Also, the screened compound may be a candidate for a compound that can inhibit interaction between a polypeptide and its associated molecules (including receptors) in vivo. The polypeptides of this invention, recepters that bind to those polypeptides or ligands, and compounds thereof, can be applied in the development of preventative, therapeutic or testing agents for diseases in which the polypeptides of the present invention are involved.

Secretory proteins may regulate cellular conditions such as growth and differentiation. A novel factor that regulates cellular conditions can be found by i) adding a secretory protein of the present invention to a certain kind of cell, and ii) screening using as an index cellular changes in growth or differentiation, or activation of a particular gene.

Screening can be performed, for example, as follows. First, a polypeptide of the present invention is expressed and purified in a recombinant form. Then, the purified polypeptide is added to various cell lines or primary cultured cells, and changes in cell growth and differentiation are monitored. The induction of a particular gene known to be involved in a certain cellular change is detected using mRNA and polypeptide amounts. Alternatively, detection can be carried out using the amount of an intracellular molecule (low-molecular-weight compounds, etc.) changed by the action of a gene product (polypeptide) which influences a certain cellular change.

If screening reveals that a polypeptide of the present invention can regulate cellular conditions or functions, it is possible to apply that polypeptide as a pharmaceutical and diagnostic medicine for related diseases, either directly or by altering a part of it into an appropriate composition.

As for membrane proteins as described above, a secretory protein provided by the present invention may be used to explore a novel ligand-receptor interaction by screening based on binding activity to a known or new ligand or receptor. A similar method can be used to identify an agonist or antagonist. Compounds obtained by these methods are candidate compounds for inhibiting the interaction between the polypeptide of the present invention and an interacting molecule (including receptors). These compounds may be applied as preventive, therapeutic, or testing agents for diseases in which the polypeptide plays a role.

Proteins involved in signal transduction or transcription may be factors that affect a certain polypeptide or gene in response to intra- or extra-cellular stimuli. A novel factor able to affect a polypeptide or gene can be found by expressing a polypeptide provided by the present invention in a certain type of cell, and screening using as an index the activation of a certain intracellular polypeptide or gene.

Screening may be performed as follows. First, a transformed cell line which expresses a polypeptide of the present invention is obtained. Then, changes in a certain gene between the transformed and the original untransformed cell lines are detected using mRNA or polypeptide amounts. Alternatively, detection may be carried out using the amount of an intracellular molecule (low molecular weight compounds, etc.) changed by the function of a certain gene product (polypeptide). Furthermore, change in expression of a certain gene can be detected by estimating the activity of a marker gene product (polypeptide), where the polypeptide of the present invention is expressed in a cell that has been introduced with a fusion gene comprising a regulatory region of the certain gene and a marker gene (luciferase, β-galactosidase, etc.).

If the polypeptide or gene influenced by a protein of the present invention is involved in disease, it is possible to screen a gene or compound that can regulate that polypeptide or gene's expression and/or activity, either directly or indirectly, by utilizing a polypeptide of the present invention.

For example, a polypeptide of the present invention is expressed and purified as a recombinant polypeptide, and a polypeptide or gene that interacts with that polypeptide is purified and screened based on binding. Alternatively, changes in binding activity can be monitored after adding a candidate inhibitor compound. In another method, the 5′-upstream transcription regulatory region of a gene that encodes a polypeptide of the present invention and that can regulate the expression of another gene, is obtained. The gene is fused with a marker gene and then introduced into a cell, compounds and the like are added, and a factor which can regulate expression of that gene can be discovered.

A compound obtained by this screening can be used for developing pharmaceutical medicines for a disease in which a polypeptide of the present invention is involved. Similarly, if a regulatory factor obtained by screening turns out to be a polypeptide, compounds that can newly affect the expression or activity of this polypeptide may be used as a medicine for diseases in which the polypeptide of the present invention is involved.

If a polypeptide of the present invention has enzymatic activity, regardless of whether it is a secretory protein, membrane protein, or a protein involved in signal transduction, glycoprotein, transcription, or disease, screening may be performed by adding a compound to the polypeptide of the present invention under suitable conditions, and monitoring that compound's change. This enzymatic activity may also be used as an index to screen a compound that inhibits the polypeptide activity.

In an example of this screening, a polypeptide of the present invention is expressed and the recombinant polypeptide is purified. Then, compounds are contacted with the purified polypeptide, and the amount of compound and reaction product is examined. Alternatively, inhibitor candidate compounds are added, then a compound (substrate) that reacts with the purified polypeptide is added, and change in the amount of substrate and reaction product is examined.

A compound obtained by screening may be used as a medicine for diseases in which a polypeptide of the present invention is involved. Also such a compound can be applied in tests that examine, for example, whether a polypeptide of the invention functions normally in vivo.

Whether a secretory protein, membrane protein, signal transduction-related protein, glycoprotein-related protein, or transcription-related protein of the present invention is a novel protein involved in disease or not is determined using different method to that described above. In this method, a specific antibody against a polypeptide of the present invention is obtained, and the relationship between the expression or activity of the polypeptide and a certain disease is examined. Alternatively, analysis can use the methods in “Molecular Diagnosis of Genetic Diseases” as a reference (Elles R. edit, (1996) in the series of “Method in Molecular Biology” (Humana Press).

Proteins involved in disease are very useful in developing drugs which regulate their expression and activity, and become targets of the above-mentioned screenings. They are also useful in the medicinal industry as diagnostic markers for their related disease, or as gene therapy targets.

A compound isolated as mentioned above can be administered to patients as is, or after being formulated into a pharmaceutical composition according to known methods. Specific examples of pharmaceutically acceptable carriers or vehicles include sterilized water, saline, plant oils, emulsifiers, suspending agents and the like, where they are mixed with the compound appropriately. The pharmaceutical compositions can be administered to patients by a method known to those skilled in the art, such as intraarterial, intravenous, or subcutaneous injection. Dosage may vary depending on the weight or age of a patient, or the method of administration, but those skilled in the art can properly choose an appropriate dosage. If a compound is encoded by a polynucleotide, the polynucleotide can be cloned into a gene therapy vector, and used for gene therapy. Those skilled in the art can properly choose the dosage of the polynucleotide and the method of its administration, and these may vary depending on the weight, age or symptoms of a patient.

The present invention further relates to databases comprising at least one sequence of a polynucleotide and/or polypeptide, or a medium recorded in such databases, selected from the sequence data of the nucleotides and/or amino acids indicated in Table 1. The term “database” means a set of accumulated, machine-searchable and readable nucleotide sequence information. The databases of the present invention comprise at least one of the novel nucleotide sequences of the polynucleotides provided by the present invention. The databases of the present invention can consist only of the sequence data of the novel polynucleotides provided by the present invention, or can comprise other information on known nucleotide sequences of full-length cDNAs or ESTs. The databases of the present invention can be comprised of not only information on nucleotide sequences, but also information on gene functions as revealed by the present invention. Additional information such as the names of DNA clones carrying the full-length cDNAs can be recorded or linked together with the sequence data in the databases.

The database of the present invention is useful for gaining complete gene sequence information from the partial sequence information of a gene of interest. The database of the present invention comprises nucleotide sequence information of full-length cDNAs. Consequently, by comparing information in this database with the nucleotide sequence of a partial gene fragment yielded by differential display method or subtraction method, information on the full-length nucleotide sequence of interest can be gained from the sequence of the partial fragment as a starting clue.

Sequence information of the full-length cDNAs constituting the database of the present invention contains information on not only complete sequences, but also on gene expression frequency and gene homology to known genes and polypeptides. This extra information facilitates rapid functional analyses of partial gene fragments. Further, information on human genes is accumulated in the database of the present invention, and therefore, the database is useful for isolating a human homologue of a gene originating from another species. The human homologue can be isolated based on the nucleotide sequence of the gene from the original species.

At present, information on a wide variety of gene fragments can be obtained by differential display method and subtraction method. In general, these gene fragments are utilized as tools for isolating the full-length sequences thereof. When the gene fragment corresponds to a known gene, the full-length sequence is easily obtained by comparing the partial sequence with the information in known databases. However, when there is no information corresponding to the partial sequence of interest in known databases, cDNA cloning should be carried out for the full-length cDNA. It is often difficult to obtain the full-length nucleotide sequence using partial sequence information as an initial clue. If the full-length gene is not available, the amino acid sequence of the polypeptide encoded by that gene remains unidentified. Thus the database of the present invention can contribute to the identification of full-length cDNAs corresponding to gene fragments, which cannot be revealed using databases of known genes.

The present invention has provided 2,495 polynucleotides. In humans, where isolation of full-length cDNA has not progressed, the provision of new full-length cDNAs has great significance. Secretory proteins, membrane proteins, signal transduction-related proteins, glycoprotein-related proteins, transcription-related proteins, and, so on are known to be involved in many diseases. The genes and proteins involved in diseases are useful for developing diagnostic markers or medicines for regulation of their expression and activity, or as a gene therapy target.

In particular, cDNA encoding secretory proteins of the present invention are extremely important to the industry since these proteins are expected to be useful as pharmaceutical agents, and many disease-related genes can be included with them. In addition, membrane proteins, signal transduction-related proteins, transcription-related proteins, disease-related proteins, and genes encoding these proteins can be used as disease indexes, etc. These cDNAs are also very important to the industry, and are expected to be effective in treating diseases and the like by regulating the activity or expression of the proteins they encode.

Human cDNAs involved in various diseases, morbid states, or functions were isolated in the present invention. The cDNAs of the present invention include, for example, genes that can be useful as diagnostic markers or therapeutic targets for the diseases or morbid states shown below. These diseases receive widespread attention, and require the development of new technology for treatment and diagnosis.
osteoporosis
neurologic diseases
Alzheimer's disease
Parkinson's disease
dementia
various cancers

In addition, human cDNAs involved in the functions shown below were isolated in the present invention. These genes can be used to elucidate the mechanisms of various functions, and in therapeutic methods which enhance or repress those functions. For example, genes participating in tissue generation and functional expression can be used in regenerative medicines.
emotional reaction
tissue generation and functional expression
motor function controlled by the brain, signaling function controlled by the brain
emotional reaction, fear response, and panic

Furthermore, information on the nucleotide sequence of the full-length cDNAs or their full-length amino acid sequences as provided by the present invention can be used to isolate human genes based on partial sequence information obtained by functional analysis of various genes, or from sequence information of genes from non-human organisms.

Any patents, patent applications, and publications cited herein are incorporated by reference.

The present invention is illustrated more specifically with reference to the following examples, but is not to be construed as being limited thereto.

### EXAMPLE 1

### Preparation of cDNA Library by Oligo-Capping

(1) Extraction and Purchase of mRNA

Total RNAs as mRNA sources were extracted from human tissues (shown below) by the method described in the reference (J. Sambrook, E. F. Fritsch & T. Maniatis, Molecular Cloning Second edition, Cold Spring harbor Laboratory Press, 1989). Further, by the method described in the reference (J. Sambrook, E. F. Fritsch & T. Maniatis, Molecular Cloning Second edition, Cold Spring harbor Laboratory Press, 1989), total RNAs as mRNA sources were extracted from human culture cells and human primary culture cells (shown below) cultivated by the methods described in the catalogs.

The library names and origins are indicated below in the order of -Library name: Origin”. When a library was prepared by the subtraction method, the item is followed by a description of how to prepare the subtracted library.

<Extraction of mRNA from Human Tissues>

NTONG: Normal tongue;

CTONG: Tongue cancer;

FCBBF: Fetal brain;

OCBBF: Fetal brain;

PLACE: Placenta;

SYNOV: Synovial membrane tissue (from rheumatioid arthritis);

CORDB: Cord blood.

<Extraction of mRNA from Culture Cells>

BNGH4: H4 cells (ATCC #HTB-148);

IMR32: IMR32 cells (ATCC #CCL-127);

SKNMC: SK-N-MC cells (ATCC #HTB-10);

3NB69: NB69 cells (RCB #RCB0480);

BGGI1: GI1 cells (RCB #RCB0763);

NB9N4: NB9 cells (RCB #RCB0477);

SKNSH: SK-N-SH cells (RCB #RCB0426);

AHMSC: Human mesenchymal cells (HMSC);

CHONS: Chondrocytes;

ERLTF: TF-1 cells (Erythroleukemia);

HELAC: HeLa cells;

JCMLC: Myelogenous leukemia cells;

MESTC: Mesenchyme stem cells;

N1ESE: Mesenchymal stem cells;

NCRRM: Embryonal carcinomas;

NCRRP: Embryonal carcinomas treated with retinoic acid (RA) to induce the differentiation;

T1ESE: Mesenchymal stem cells treated with trichostatin and 5-azacytidine to induce the differentiation;

NT2RM: NT2 cells (STARATAGENE #204101);

NT2RP: NT2 cells treated with RA for 5 weeks to induce the differentiation;

NT2RI: NT2 cells treated with RA for 5 weeks to induce the differentiation, followed by the treatment with the growth inhibitor for 2 weeks;

NT2NE: NT2 cells were treated with RA and the growth inhibitor for the neuronal differentiation, and the resultant neurons were concentrated and harvested (NT2 Neuron);

NTISM: NT2 cells (STARATAGENE #204101) were treated with RA for five weeks to induce the differentiation, and then treated with the growth inhibitor for two weeks; mRNA was prepared from the cells and a cDNA library was constructed from the mRNA; the cDNA libraries whose nucleotide sequences were shared by those of mRNAs from undifferentiated NT2 cells were subtracted by using a Subtract Kit (Invitrogen #K4320-01); the subtracted library (NT2RI-NT2RM) was provided by this procedure.

RCB indicates that the cell was provided by the Cell Bank, RIKEN GENE BANK, The Institute of Physical and Chemical Research; ATCC indicates that the cell was provided by American Type Culture Collection.

<Extraction of mRNA from Primary Culture Cells>

ASTRO: Normal human astrocyte NHA5732, Takara Shuzo #CC2565;

DFNES: Normal human dermal fibroblast (neonatal skin); NHDF-Neo NHDF2564, Takara Shuzo #CC2509;

MESAN: Normal human mesangial cell NHMC56046-2, Takara Shuzo #CC2559;

NHNPC: Normal human neural progenitor cell NHNP5958, Takara Shuzo #CC2599;

PEBLM: Normal human peripheral blood mononuclear cell HPBMC5939, Takara Shuzo #CC2702;

HSYRA: Human synoviocyte HS-RA (from rheumatoid arthritis), Toyobo #T404K-05;

PUAEN: Normal human pulmonary artery endothelial cells, Toyobo #T302K-05;

UMVEN: Normal human umbilical vein endothelial cell HUVEC, Toyobo #T200K-05;

HCASM: Normal human coronary artery smooth muscle cell HCASMC, Toyobo #T305K-05;

HCHON: Normal human chondrocyte HC, Toyobo #T402K-05;

HHDPC: Normal human dermal papilla cell HDPC, Toyobo #THPCK-001;

CD34C: CD34+ cells (AllCells, LLC #CB14435M);

D3OST: CD34+ cells treated with the osteoclast differentiation factor (ODF) for three days to induce the differentiation;

D6OST: CD34+ cells treated with ODF for six days to induce the differentiation;

D9OST: CD34+ cells treated with ODF for nine days to induce the differentiation;

ACTVT: Activated T-cells;

LYMPB: Lymphoblasts (EB virus transferred B cells);

NETRP: Neutrophils.

Total RNAs extracted from the following human tissues were then purchased and used as mRNA sources. Library names and the origins are indicated below in the order of “Library name: Origin”. When a library was prepared by the subtraction method, the item is followed by a description of how to prepare the subtracted library.

<Purchase of Total RNA Containing mRNA Extracted from Human Tissues>

ADRGL: Adrenal gland, CLONTECH #64016-1;

BRACE: Brain (cerebellum), CLONTECH #64035-1;

BRAWH: Whole brain, CLONTECH #64020-1;

FEBRA: Fetal brain, CLONTECH #64019-1;

FELIV: Fetal liver, CLONTECH #64018-1;

HEART: Heart, CLONTECH #64025-1;

HLUNG: Lung, CLONTECH #64023-1;

KIDNE: Kidney, CLONTECH #64030-1;

LIVER: Liver, CLONTECH #64022-1;

MAMGL: Mammary Gland, CLONTECH #64037-1;

PANCR: Pancreas, CLONTECH #64031-1;

PROST: Prostate, CLONTECH #64038-1;

SALGL: Salivary Gland, CLONTECH #64026-1;

SKMUS: Skeletal Muscle, CLONTECH #64033-1;

SMINT: Small Intestine, CLONTECH #64039-1;

SPLEN: Spleen, CLONTECH #64034-1;

STOMA: Stomach, CLONTECH #64090-1;

TBAES: Breast (Tumor), CLONTECH #64015-1;

TCERX: Cervix (Tumor), CLONTECH #64010-1;

TCOLN: Colon (Tumor), CLONTECH #64014-1;

TESTI: Testis, CLONTECH #64027-1;

THYMU: Thymus, CLONTECH #64028-1;

TLUNG: Lung (Tumor), CLONTECH #64013-1;

TOVAR: Ovary (Tumor), CLONTECH #64011-1;

TRACH: Trachea, CLONTECH #64091-1;

TUTER: Uterus (Tumor), CLONTECH #64008-1;

UTERU: Uterus, CLONTECH #64029-1;

ADIPS: Adipose, Invitrogen #D6005-01;

BLADE: Bladder, Invitrogen #D6020-01;

BRALZ: Cerebral cortex from an Alzheimer patient (Brain, cortex, Alzheimer), Invitrogen #D6830-01;

CERVX: Cervix, Invitrogen #D6047-01;

COLON: Colon, Invitrogen #D6050-0;

NESOP: Esophagus, Invitrogen #D6060-01;

PERIC: Pericardium, Invitrogen #D6105-01;

RECTM: Rectum, Invitrogen #D6110-01;

TESOP: Esophageal (Tumor), Invitrogen #D6860-01;

TKIDN: Kidney (Tumor), Invitrogen #D6870-01;

TLIVE: Liver (Tumor), Invitrogen #D6880-01;

TSTOM: Stomach (Tumor), Invitrogen #D6920-01;

BEAST: Adult breast, STARATAGENE #735044;

FEHRT: Fetal heart, STARATAGENE #738012;

FEKID: Fetal kidney, STARATAGENE #738014;

FELNG: Fetal lung, STARATAGENE #738020;

NOVAR: Adult ovary, STARATAGENE #735260;

BRASW: subtracted library (BRALZ-BRAWH). A cDNA library was constructed from mRNA prepared from tissues of cerebral cortex obtained from an Alzheimer patient [BRALZ: Cerebral cortex from an Alzheimer patient (Brain, cortex, Alzheimer), Invitrogen #D6830-01]; the cDNA libraries whose nucleotide sequences were shared by those of mRNAs from whole brain tissue [BRAWH: Whole brain, CLONTECH #64020-1] were subtracted using a Subtract Kit (Invitrogen #K4320-01).

Further, mRNAs extracted and purified as poly A(+) RNAs from the human tissues shown below were purchased. A cDNA library was prepared from an RNA mixture in which the poly A(+) RNA from each tissue was combined with poly A(−) RNA. The poly A(−) RNA was prepared by removing poly A(+) RNA from the total RNA of whole brain tissue (CLONTECH #64020-1) by using oligo dT cellulose. The library names and origins are indicated below in the order of “Library name: Origin”.

<Purchase of mRNAs of Human Tissues as Poly A(+) RNAs>

BRAMY: Brain (amygdala), CLONTECH #6574-1;

BRCAN: Brain (caudate nucleus), CLONTECH #6575-1;

BRCOC: Brain (corpus callosum), CLONTECH #6577-1;

BRHIP: Brain (hippocampus), CLONTECH #6578-1;

BRSSN: Brain (substantia nigra), CLONTECH #6580-1;

BRSTN: Brain (subthalamic nucleus), CLONTECH #6581-1;

BRTHA: Brain (thalamus), CLONTECH #6582-1.

(2) Preparation of cDNA Libraries

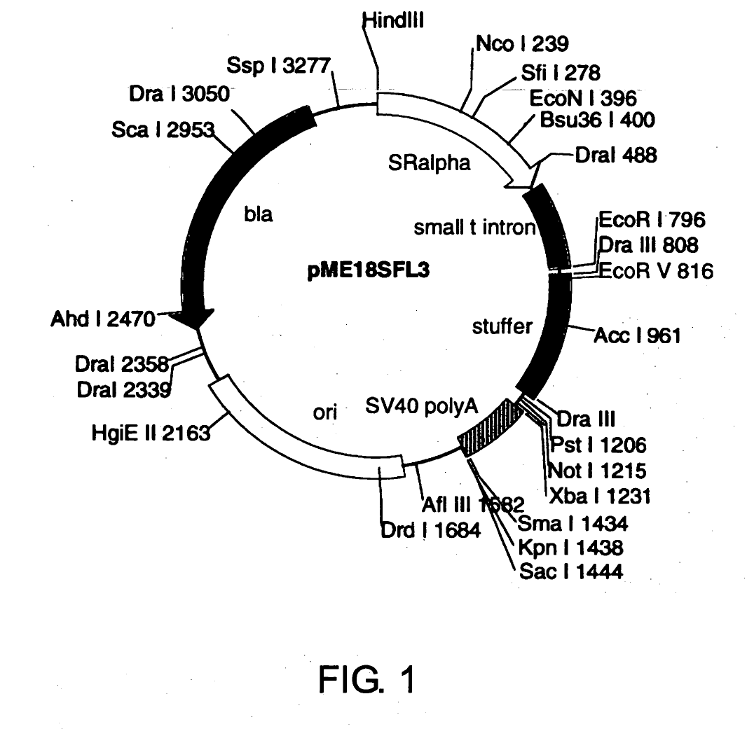

A cDNA library was prepared from each RNA using the improved method (WO 01/04286) of oligo capping [M. Maruyama and S. Sugano, Gene, 138: 171-174 (1994)]. A series of procedures, BAP (Bacterial Alkaline Phosphatase) treatment, TAP (Tobacco Acid Pyrophosphatase) treatment, RNA ligation, first strand cDNA synthesis and RNA removal, were carried out using the oligo-cap linker (agcaucgagu cggccuuguu ggccuacugg/SEQ ID NO: 4991) and oligo dT primer (gcggctgaag acggcctatg tggccttttt tttttttttt tt/SEQ ID NO: 4992), as described in WO 01/04286. The single-stranded cDNA was then converted to a double-stranded cDNA by PCR (polymerase chain reaction) using 5′ (agcatcgagt cggccttgtt g/SEQ ID NO: 4993) and 3′ (gcggctgaag acggcctatg t/SEQ ID NO: 4994) PCR primers, and then digested with SfiI. Then, a fraction of cDNA fragments, typically 2-kb or longer (3-kb or longer in some cases), was unidirectionally cloned into a DraIII-digested pME18SFL3 vector (FIG. 1) (GenBank AB009864, Expression vector); and the cDNA library was thus prepared.

Shown below are the names of cDNA libraries used in the analysis of full-length cDNA sequences, and their origins. The Library Name is provided with the Type, Origin, and such of the library source, demarcated by a slash mark (/) within parentheses.

3NB69 (culture cells/NB69 cells (RCB #RCB0480))

ACTVT (primary culture cells/Activated T-cells (Activated T-cell))

ADIPS (Tissues/Adipose (Invitrogen #D6005-01))

ADRGL (Tissues/Adrenal gland (CLONTECH #64016-1))

ASTRO (primary culture cells/Normal Human Astrocyte NHA5732 (Takara Shuzo #CC2565))

BLADE (Tissues/Bladder (Invitrogen #D6020-01))

BNGH4 (culture cells/H4 cells (ATCC #HTB-148))

BRACE (Tissues/Brain (cerebellum) (CLONTECH #64035-1))

BRALZ (Tissues/Cerebral cortex from an Alzheimer patient (Brain, cortex, Alzheimer) (Invitrogen #D6830-01))

BRAMY (Tissues/Brain (amygdala) (CLONTECH #6574-1))

BRASW (Tissues/subtracted library (BRALZ-BRAWH). The cDNAs from tissues of cerebral cortex obtained from an Alzheimer patient whose nucleotide sequences were shared by those of mRNAs from whole brain tissue were subtracted.

BRAWH (Tissues/Whole brain (CLONTECH #64020-1))

BRCAN (Tissues/Brain (caudate nucleus) (CLONTECH #6575-1))

BRCOC (Tissues/Brain (corpus callosum) (CLONTECH #6577-1))

BRHIP (Tissues/Brain (hippocampus) (CLONTECH #6578-1))

BRSSN (Tissues/Brain (substantia nigra) (CLONTECH #6580-1))

BRSTN (Tissues/Brain (subthalamic nucleus) (CLONTECH #6581-1))

BRTHA (Tissues/Brain (thalamus) (CLONTECH #6582-1))

CERVX (Tissues/Cervix (Invitrogen #D6047-01))

COLON (Tissues/Colon (Invitrogen #D6050-0))

CORDB (Tissues/Cord blood)

CTONG (Tissues/Tongue Cancer)

D3OST (primary culture cells/CD34+ cells (treated with ODF for three days to induce the differentiation))

DFNES (primary culture cells/Normal human dermal fibroblasts (neonatal skin); NHDF-Neo NHDF2564 (Takara Shuzo #CC2509))

ERLTF (culture cells/TF-1 cells (Erythroleukemia))

FCBBF (Tissues/Fetal brain)

FEBRA (Tissues/Fetal brain (CLONTECH #64019-1))

HCASM (primary culture cells/Normal human coronary artery smooth muscle cell HCASMC (Toyobo #T305K-05))

HCHON (primary culture cells/Normal human chondrocyte HC (Toyobo #T402K-05))

HEART (Tissues/Heart (CLONTECH #64025-1))

HHDPC (primary culture cells/Normal human dermal papilla cell HDPC (Toyobo #THPCK-001))

HLUNG (Tissues/Lung (CLONTECH #64023-1))

HSYRA (primary culture cells/Human synoviocyte HS-RA (from rheumatoid arthritis)(Toyobo #T404K-05))

IMR32 (culture cells/IMR32 cells (ATCC #CCL-127))

KIDNE (Tissues/Kidney (CLONTECH #64030-1))

LIVER (Tissues/Liver (CLONTECH #64022-1))

LYMPB (primary culture cells/Lymphoblasts (EB virus transferred B cells))

MESAN (primary culture cells/Normal human mesangial cell NHMC56046-2 (Takara Shuzo #CC2559))

MESTC (culture cells/Mesenchyme stem cells)

NB9N4 (culture cells/NB9 cells (RCB #RCB0477))

NCRRP (culture cells/Embryonal carcinomas treated with RA to induce the differentiation)

NESOP (Tissues/Esophagus (Invitrogen #D6060-01))

NHNPC (primary culture cells/Normal human neural progenitor cell NHNP5958 (Takara Shuzo #CC2599))

NOVAR (Tissues/Adult ovary (STARATAGENE #735260))

NT2NE (culture cells/NT2 cells were treated with RA and the growth inhibitor for the neuronal differentiation, and the resultant neurons were concentrated and harvested (NT2 Neuron))

NT2RI (culture cells/NT2 cells treated with RA for five weeks to induce the differentiation, followed by the treatment with the growth inhibitor for 2 weeks)

NT2RP (culture cells/NT2 cells treated with RA for five weeks to induce the differentiation)

NTONG (Tissues/Normal tongue)

OCBBF (Tissues/Fetal brain)

PEBLM (primary culture cells/Normal human peripheral blood mononuclear cell HPBMC5939 (Takara Shuzo #CC2702))

PERIC (Tissues/Pericardium (Invitrogen #D6105-01))

PLACE (Tissues/Placenta)

PROST (Tissues/Prostate (CLONTECH #64038-1))

PUAEN (primary culture cells/Normal human pulmonary artery endothelial cells (Toyobo #T302K-05))

RECTM (Tissues/Rectum (Invitrogen #D6110-01))

SKMUS (Tissues/Skeletal Muscle (CLONTECH #64033-1))

SKNMC (culture cells/SK-N-MC cells (ATCC #HTB-10))

SKNSH (culture cells/SK-N-SH cells (RCB #RCB0426))

SMINT (Tissues/Small Intestine (CLONTECH #64039-1))

SPLEN (Tissues/Spleen (CLONTECH #64034-1))

STOMA (Tissues/Stomach (CLONTECH #64090-1))

SYNOV (Tissues/Synovial membrane tissue (from rheumatoid arthritis))

T1ESE (culture cells/Mesenchymal stem cell (treated with trichostatin and 5-azacytidine to induce the differentiation))

TBAES (Tissues/Breast (Tumor) (CLONTECH #64015-1))

TCERX (Tissues/Cervix (Tumor) (CLONTECH #64010-1))

TCOLN (Tissues/Colon (Tumor) (CLONTECH #64014-1))

TESOP (Tissues/Esophageal (Tumor) (Invitrogen #D6860-01))

TESTI (Tissues/Testis (CLONTECH #64027-1))

THYMU (Tissues/Thymus (CLONTECH #64028-1))

TKIDN (Tissues/Kidney (Tumor) (Invitrogen #D6870-01))

TLIVE (Tissues/Liver (Tumor) (Invitrogen #D6880-01))

TOVAR (Tissues/Ovary (Tumor) (CLONTECH #64011-1))

TRACH (Tissues/Trachea (CLONTECH #64091-1))

TSTOM (Tissues/Stomach (Tumor) (Invitrogen #D6920-01))

TUTER (Tissues/Uterus (Tumor) (CLONTECH #64008-1))

UTERU (Tissues/Uterus (CLONTECH #64029-1))

cDNA libraries with a high fullness ratio (the fullness ratio of 5′-end was 90% on average, calculated for each cDNA library using the protein coding region found in known mRNA species as an index) prepared by the improved oligo-capping method were constructed using a eukaryotic expression vector pME18SFL3. The vector contained SRα promoter and SV40 small t intron upstream of the cloning site, and SV40 polyA added signal sequence site downstream. As the cloning site of pME18SFL3 has asymmetrical DraIII sites, and the ends of cDNA fragments contain SfiI sites complementary to the DraIII sites, the cloned cDNA fragments can be inserted downstream of the SRα promoter unidirectionally. Therefore, clones containing full-length cDNA can be expressed transiently by introducing the obtained plasmid directly into COS cells, etc. Thus, clones can be analyzed very easily using the clone gene product proteins, or those proteins' biological activities.

(3) Assessment of 5′-End Completeness of Clones Derived from a cDNA Library Prepared by Oligo-Capping

With respect to the plasmid DNAs of clones derived from the libraries, the nucleotide sequences of cDNA 5′-ends (3′-ends as well in some cases) were determined in a DNA sequencer (ABI PRISM 3700, PE Biosystems), after sequencing reaction were conducted using a DNA sequencing reagent (BigDye Terminator Cycle Sequencing FS Ready Reaction Kit, PE Biosystems) according to the manual. A database was constructed based on the obtained data.

The 5′-end completeness of about 1,110,000 clones derived from the human cDNA libraries prepared by the improved oligo-capping method was determined using the following method. The clones whose 5′-end sequences were consistent with those of known human mRNA in the public database were judged to be “full-length” if they had a longer 5′-end sequence than that of the known human mRNA. Even if the 5′-end sequence was shorter, clones containing the translation initiation codon were judged to comprise the “full-length” sequence. Clones which did not comprise the translation initiation codon were judged to be “not-full-length”. The fullness ratio ((the number of full-length clones)/(the number of full-length and not-full-length clones)) at the 5′-end of the cDNA clones was determined by comparison with known human mRNA. The fullness ratio of the 5′-ends was found to be 90%. The result indicates that the fullness ratio at the 5′-end sequence was extremely high in human cDNA clones obtained by the oligo-capping method.

### EXAMPLE 2

### Sequencing Analysis of cDNA Ends and Selection of Full-Length Clones

With respect to the plasmid DNAs of clones obtained from each cDNA library, the 5′-end nucleotide sequences of the cDNAs were determined in a DNA sequencer (ABI PRISM 3700, PE Biosystems), after sequencing reaction was conducted by using a DNA sequencing reagent (Dye Terminator Cycle Sequencing FS Ready Reaction Kit, dRhodamine Terminator Cycle Sequencing FS Ready Reaction Kit or BigDye Terminator Cycle Sequencing FS Ready Reaction Kit, PE Biosystems) according to the manual. A database was constructed using the data obtained.

For the analyzed 5′-end sequences of cDNA clones, the data with the annotation of “complete cds” in the GenBank and UniGene were searched by BLAST homology search. When identical to certain human mRNA sequences, such cDNA clones were excluded. Then, clustering was carried out. When the identity was 90% or higher, and the length of consensus sequence was 50 base pairs or longer, the cDNA clones were assumed to belong to an identical cluster, and thus clustered. cDNA clones longer in the 5′ direction were selected from the members belonging to a cluster; if required, the 3′-end sequences of the selected clones were determined by the same analysis method as used to determine the 5′-end sequences. The data of the end sequences obtained were analyzed, and then the clones forming a sequence contig at 5′- and 3′-ends were excluded. Further, as mentioned above, the data was analyzed again by BLAST homology search; when identical to certain human mRNA sequences (including sequences patented and applied for), the cDNA clones were excluded. Thus, the cDNAs clones to be analyzed for their nucleotide sequence were obtained.

### EXAMPLE 3

### Analysis of the Full-Length Nucleotide Sequences

The full-length nucleotide sequences of the selected clones were determined. The nucleotide sequence determination was mainly performed by primer walking method comprising the dideoxy terminator method using custom-made synthetic DNA primers. Namely, the nucleotide sequences of the DNAs were determined in a sequencer from PE Biosystems, after sequencing reaction was carried out with a DNA sequencing reagent from the same supplier using the custom-made synthetic DNA primers according to the manual. A part of the clones were analyzed with a DNA sequencer from Licor.

Further, the nucleotide sequences of a part of the clones were determined by the shotgun method where the plasmids containing the cDNAs were digested at random were used, instead of the use of custom-made primers, by the same method in the DNA sequencer. The full-length nucleotide sequences were finally determined by completely assembling the partial nucleotide sequences obtained by the above method.

Then, the regions translatable to proteins were deduced from the determined full-length nucleotide sequences, and thereby the amino acid sequences were determined. SEQ ID NOs corresponding to the respective sequences are shown in Table 1.

### EXAMPLE 4

### Functional Prediction by Homology Search

GenBank, SwissProt, UniGene, RefSeq, and nr were searched using BLAST for the determined nucleotide sequences (SEQ ID NOs: 1-2188) and the ORF amino acid sequences deduced to encode the polypeptides (SEQ ID NOs: 2189-4376). SwissProt, RefSeq, and nr were searched using BLAST for the nucleotide sequences of SEQ ID NOs: 4377-4683, and the amino acid sequences of SEQ ID NOs: 4684-4990. Of the hit data which met the criteria described below, representative hit data were selected. This representative data is hit data with a higher homology and that enables relatively easy functional prediction for nucleotide sequences and deduced amino acid sequences. The results of the homology search are shown at the end of this specification.

Hit data whose P value or E value is 10⁻⁴ or less

For analysis using an amino acid database, hit data whose P value or E value is 10⁻⁴ or less, where the length of consensus sequence×homology=30 or higher,

Thus, only representative data are indicated and molecules exhibiting homology to each clone are not limited thereto. For some clones, hit data that do not meet the above-described criteria in BLAST search are not shown.

### EXAMPLE 5

### Search for Signal Sequence, Transmembrane Domain and Other Functional Domains in the Deduced Amino Acid Sequences

With respect to the amino acid sequences deduced from the full-length nucleotide sequences, the prediction was made for the presence of signal sequence at the amino terminus, the presence of transmembrane domain, and the presence of functional protein domains (motifs). The signal sequence at the amino terminus was searched for by PSORT [K. Nakai & M. Kanehisa, Genomics, 14: 897-911 (1992)]; the transmembrane domain, by SOSUI [T. Hirokawa et al., Bioinformatics, 14: 378-379 (1998)] (Mitsui Knowledge Industry); the function domain, by Pfam (Version 5.5) (http://www.sanger.ac.uk/Software/Pfam/index.shtml). The amino acid sequence in which the signal sequence at the amino terminus or transmembrane domain had been predicted to be present by PSORT or SOSUI were assumed to be a secretory or membrane protein. Further, when the amino acid sequence hit a certain functional domain by the Pfam functional domain search, the protein function can be predicted based on the hit data, for example, by referring to the function categories on the PROSITE (http://www.expasy.ch/cgi-bin/prosite-list.pl). In addition, the functional domain search can also be carried out on the PROSITE.

The search results obtained with the respective programs are shown below.

The 161 clones whose deduced amino acid sequences were detected to have the signal sequences by PSORT are as follows.

ADIPS2000088, ADRGL2000172, ADRGL2009146, BNGH42003570, BRACE2030341, BRACE2031531, BRACE2039327, BRACE2041200, BRACE2043142, BRACE3004113, BRACE3004843, BRACE3010397, BRACE3011505, BRACE3026735, BRACE3040856, BRAMY2004771, BRAMY2005052, BRAMY2019300, BRAMY2019963, BRAMY3007206, BRAMY3007609, BRAMY3008505, BRAWH2002560, BRAWH3001475, BRAWH3003992, BRAWH3004666, BRAWH3006548, BRCAN2002948, BRCAN2010376, BRCAN2012481, BRHIP2005236, BRHIP2026288, BRHIP3008598, BRSSN2014424, BRSTN2007000, BRSTN2010363, BRSTN2016470, BRTHA2002608, BRTHA2005579, BRTHA2016496, BRTHA2018344, BRTHA3000633, BRTHA3017047, COLON2000568, COLON2002443, COLON2005126, CTONG1000302, CTONG1000488, CTONG1000508, CTONG2000042, CTONG2008233, CTONG3000707, CTONG3001560, CTONG3003179, CTONG3003483, CTONG3003737, CTONG3005648, CTONG3008258, CTONG3009385, D3OST2002182, D3OST2002648, FCBBF2001183, FCBBF2007510, FCBBF3009888, FCBBF4000076, FEBRA2025427, HCASM2002502, HCHON2001577, HCHON2002676, HEART2001680, HSYRA2009102, IMR322002110, KIDNE2000846, KIDNE2006580, KIDNE2013095, LYMPB2000083, NOVAR2000136, NOVAR2001108, NT2RI2008724, NT2RI3005403, NT2RI3007065, NT2RP7000359, NT2RP7000466, NTONG2000413, OCBBF2006764, PLACE5000171, PLACE5000282, PLACE6012574, PROST2018090, PUAEN2002489, PUAEN2009795, RECTM2000433, SKMUS2000757, SKNMC2002402, SMINT2015787, SPLEN2009548, SPLEN2012624, SPLEN2012889, SPLEN2021701, SPLEN2030479, SPLEN2031125, SPLEN2034021, SPLEN2034781, SPLEN2037722, SPLEN2040222, STOMA2004294, SYNOV4002883, SYNOV4008336, TESOP2002273, TESOP2005485, TESOP2007636, TESTI2002618, TESTI2036684, TESTI2048898, TESTI4001561, TESTI4001665, TESTI4008401, TESTI4008797, TESTI4014392, TESTI4020102, TESTI4025797, TESTI4028429, TESTI4035065, TESTI4035649, TESTI4042711, TESTI4046487, THYMU2001053, THYMU2003632, THYMU2005321, THYMU2013386, THYMU2014353, THYMU2019210, THYMU2027497, THYMU2027695, THYMU2029676, THYMU2039780, THYMU2040412, THYMU3004835, THYMU3005696, THYMU3007845, TKIDN2002424, TKIDN2019116, TLIVE2002690, TRACH2007834, TRACH3002650, TRACH3004786, TRACH3006149, TRACH3035199, TRACH3035526, UTERU1000339, UTERU2025645, UTERU2026090, UTERU3000645, UTERU3000899, UTERU3001585, UTERU3002209, UTERU3002383, UTERU3003776, UTERU3009690, UTERU3009979, UTERU3015500

Deduced amino acid sequences of following 20 clones were also detected to have the signal sequences by PSORT.

BRACE2002589, BRACE2009318, BRACE2039823, BRAWH2006395, BRAWH2008993, BRCOC2019841, BRHIP2005271, BRTHA2011321, FEBRA2028256, HCASM2003099, PROST2000452, PROST2019487, SPLEN2016932, STOMA2003158, SYNOV2001660, SYNOV4003981, TESTI2015626, TESTI4000319, TKIDN2018926, UTERU2032279

The 523 clones whose deduced amino acid sequences were detected to have the transmembrane domains by SOSUI are as follows. Numerals indicate the numbers of transmembrane domains detected in the deduced amino acid sequences. Of the search result, the Clone Name and the Number of transmembrane domains are demarcated by a double slash mark (//).

ACTVT2000380//1, ADRGL2003329//2, ASTRO2014923//6, ASTRO3000301//1, BLADE1000176//1, BLADE2002073//1, BLADE2002947//2, BLADE2004462//2, BLADE2004670//1, BLADE2008539//1, BNGH42003570//1, BRACE1000186//1, BRACE2005457//8, BRACE2014306//11, BRACE2016981//1, BRACE2030341//1, BRACE2030884//3, BRACE2031527//1, BRACE2031899//2, BRACE2032385//3,

BRACE2036005//1, BRACE2040138//2, BRACE2043142//4, BRACE2043665//1, BRACE3000697//4, BRACE3001391//3, BRACE3002298//1, BRACE3003004//1, BRACE3004113//1, BRACE3004843//1, BRACE3006462//3, BRACE3008384//6, BRACE3009574//1, BRACE3009708//6, BRACE3010397//2, BRACE3011505//2, BRACE3013740//3, BRACE3014005//8, BRACE3014068//3, BRACE3014807//3,

BRACE3020286//3, BRACE3020594//2, BRACE3024662//1, BRACE3025531//1, BRACE3026008//2, BRACE3031838//1, BRACE3040856//3, BRALZ2016085//10, BRAMY2004771//2, BRAMY2005052//2, BRAMY2017528//1, BRAMY2019300//2, BRAMY2019963//1, BRAMY2028856//2, BRAMY2033003//2, BRAMY2033116//2, BRAMY2033594//1, BRAMY2036396//2, BRAMY2039872//2, BRAMY2040592//2,

BRAMY2041542//1, BRAMY2045036//1, BRAMY2047420//1, BRAMY2047765//3, BRAMY3002312//1, BRAMY3004224//2, BRAMY3004919//5, BRAMY3008505//2, BRASW1000125//2, BRAWH2002560//1, BRAWH2002761//3, BRAWH2007658//2, BRAWH2014414//1, BRAWH2016439//1, BRAWH2016702//3, BRAWH3000078//2, BRAWH3000314//1, BRAWH3001891//6, BRAWH3002600//1, BRAWH3003555//2,

BRAWH3003727//1, BRAWH3004453//1, BRAWH3004666//2, BRAWH3005132//1, BRAWH3005912//1, BRAWH3006548//2, BRAWH3007221//2, BRAWH3007506//2, BRAWH3007592//1, BRAWH3008634//1, BRCAN2012355//5, BRCAN2012481//1, BRCAN2013655//3, BRCAN2014143//5, BRCAN2028355//4, BRCOC2007034//2, BRCOC2019934//2, BRHIP2000691//1, BRHIP2001805//1, BRHIP2002172//8,

BRHIP2004814//1, BRHIP2004883//2, BRHIP2005752//2, BRHIP2009414//7, BRHIP2013699//1, BRHIP2026288//2, BRHIP3000526//1, BRHIP3007483//2, BRHIP3007586//1, BRHIP3008598//3, BRHIP3015751//1, BRHIP3024118//7, BRHIP3026097//1, BRSSN2003086//1, BRSSN2008549//1, BRSSN2011738//2, BRSSN2014424//9, BRSSN2018925//2, BRSTN2003835//1, BRSTN2007000//1,

BRSTN2012380//1, BRSTN2015015//2, BRSTN2016678//3, BRSTN2017110//2, BRTHA2002281//2, BRTHA2002376//2, BRTHA2002493//1, BRTHA2002608//2, BRTHA2002808//1, BRTHA2003110//2, BRTHA2003461//3, BRTHA2006075//2, BRTHA2011194//1, BRTHA2012980//2, BRTHA2013460//2, BRTHA2015696//2, BRTHA2015878//2, BRTHA2016215//1, BRTHA2017985//1, BRTHA2018344//3,

BRTHA2018624//1, BRTHA3000633//2, BRTHA3002427//12, BRTHA3003474//2, BRTHA3007148//3, BRTHA3008386//4, BRTHA3008778//1, BRTHA3009090//1, BRTHA3009291//2, BRTHA3016845//2, BRTHA3017047//2, BRTHA3017589//2, BRTHA3017848//8, BRTHA3018656//9, CERVX2002006//1, COLON2002443//1, COLON2005126//2, CTONG1000302//1, CTONG1000341//1, CTONG2004062//4,

CTONG2008233//2, CTONG2009423//3, CTONG2009531//1, CTONG2013178//1, CTONG2019652//1, CTONG2019788//1, CTONG2020127//1, CTONG2020522//1, CTONG2020638//6, CTONG2022601//2, CTONG2023512//2, CTONG2026920//1, CTONG2027327//1, CTONG2028124//3, CTONG2028687//2, CTONG3001560//4, CTONG3002020//2, CTONG3002412//3, CTONG3003483//2, CTONG3003737//1,

CTONG3008252//1, CTONG3008496//2, CTONG3008566//1, CTONG3008951//2, CTONG3009227//1, CTONG3009239//3, CTONG3009328//5, CTONG3009385//4, D3OST2002648//6, DFNES1000107//1, DFNES2010502//3, FCBBF2001183//2, FCBBF2007510//2, FCBBF3003435//1, FCBBF3004502//10, FCBBF3009888//1, FCBBF3012170//1, FCBBF3021576//1, FCBBF3023895//1, FEBRA2007544//1,

FEBRA2007708//13, FEBRA2008311//7, FEBRA2020668//2, FEBRA2025427//1, FEBRA2027082//1, HCASM2003212//1, HCASM2007047//9, HCHON2000212//1, HCHON2001084//12, HCHON2001548//1, HCHON2001712//9, HCHON2004007//3, HCHON2005921//4, HLUNG2000014//1, HLUNG2003872//5, HLUNG2010464//4, HLUNG2015617//2, HLUNG2017350//4, HSYRA2005496//2, HSYRA2006873//1,

HSYRA2008714//6, HSYRA2009102//10, IMR322000127//2, IMR322002110//1, IMR322006222//1, KIDNE1000064//10, KIDNE2000832//10, KIDNE2000846//5, KIDNE2006580//1, KIDNE2010264//1, KIDNE2011635//12, KIDNE2012945//1, KIDNE2013095//3, LIVER2007415//1, LYMPB2000083//3, MESAN2001979//3, MESAN2012054//2, MESTC1000042//1, NHNPC2000606//1, NHNPC2001223//1,

NT2RI2008724//1, NT2RI2009855//3, NT2RI3001263//1, NT2RI3003095//3, NT2RI3003382//1, NT2RI3003409//1, NT2RI3005403//2, NT2RI3006673//1, NT2RI3007065//3, NT2RI3007543//2, NT2RI3007978//2, NT2RP7000466//1, NT2RP7009030//1, NT2RP7014005//2, NTONG2000413//1, OCBBF2006151//5, OCBBF2006567//1, OCBBF2006764//1, OCBBF2007114//1, OCBBF2007428//1,

OCBBF2009926//2, OCBBF2010140//13, OCBBF2017516//2, OCBBF2021788//1, OCBBF2024719//1, OCBBF2025458//2, OCBBF2030517//2, OCBBF2030574//3, OCBBF2031167//1, OCBBF2033869//2, OCBBF2038317//2, OCBBF3000483//1, OCBBF3003320//6, OCBBF3004314//1, PEBLM2000170//1, PEBLM2000338//2, PEBLM2002594//2, PEBLM2006113//1, PEBLM2007834//1, PERIC2001227//1,

PERIC2003452//3, PERIC2004909//2, PERIC2006035//7, PERIC2007914//3, PLACE5000171//1, PLACE5000260//2, PLACE6012574//2, PLACE7000514//1, PLACE7001022//1, PROST1000184//2, PROST1000528//2, PROST1000559//1, PROST2018902//1, PROST2018922//1, PUAEN2002489//4, PUAEN2005588//1, PUAEN2006701//3, PUAEN2009174//1, PUAEN2009852//1, RECTM2001347//2,

SMINT1000192//1, SMINT2002743//2, SMINT2009902//4, SMINT2015787//2, SPLEN2001599//1, SPLEN2009548//2, SPLEN2012889//3, SPLEN2015158//1, SPLEN2015679//1, SPLEN2021701//2, SPLEN2023733//7, SPLEN2023791//1, SPLEN2025491//1, SPLEN2029522//1, SPLEN2029683//2, SPLEN2030335//1, SPLEN2030479//1, SPLEN2031125//2, SPLEN2031424//2, SPLEN2031547//6,

SPLEN2031724//3, SPLEN2031780//2, SPLEN2032813//2, SPLEN2033098//1, SPLEN2036326//4, SPLEN2037722//2, SPLEN2038180//2, SPLEN2038345//1, SPLEN2040222//4, SPLEN2041304//1, SPLEN2042598//3, STOMA2008546//3, SYNOV2005817//2, SYNOV2012326//2, SYNOV2016124//1, SYNOV2021320//2, SYNOV4003322//3, SYNOV4004184//1, SYNOV4004741//3, SYNOV4004914//1,

SYNOV4006256//2, SYNOV4007430//1, SYNOV4007553//2, SYNOV4007671//1, SYNOV4008336//2, SYNOV4008440//4, TCERX2000613//1, TESOP2000801//1, TESOP2001345//2, TESOP2001865//2, TESOP2002273//2, TESOP2002539//3, TESOP2005579//1, TESOP2006041//1, TESOP2007052//1, TESOP2007262//1, TESOP2007636//2, TESTI1000257//11, TESTI1000348//3, TESTI2002036//6,

TESTI2002618//2, TESTI2002928//1, TESTI2003347//2, TESTI2005610//1, TESTI2006648//6, TESTI2013382//3, TESTI2024567//5, TESTI2034953//1, TESTI2034997//1, TESTI2035997//1, TESTI2042450//1, TESTI2047071//2, TESTI2048898//2, TESTI2051767//3, TESTI2052822//1, TESTI4000215//2, TESTI4000724//11, TESTI4001176//1, TESTI4001561//2, TESTI4001923//1,

TESTI4002552//4, TESTI4002754//3, TESTI4005805//1, TESTI4005961//1, TESTI4006053//1, TESTI4006137//2, TESTI4007064//3, TESTI4007163//3, TESTI4007239//1, TESTI4007382//1, TESTI4008401//1, TESTI4009608//1, TESTI4013369//3, TESTI4013667//2, TESTI4013830//3, TESTI4016238//2, TESTI4017575//2, TESTI4017901//2, TESTI4018835//2, TESTI4019566//2,

TESTI4020092//1, TESTI4020102//2, TESTI4021478//7, TESTI4023722//2, TESTI4024420//1, TESTI4024874//3, TESTI4024890//2, TESTI4026456//1, TESTI4026785//1, TESTI4027821//1, TESTI4028062//1, TESTI4028429//1, TESTI4028823//4, TESTI4028880//11, TESTI4029836//7, TESTI4030159//3, TESTI4030505//2, TESTI4034172//3, TESTI4035649//2, TESTI4037244//1,

TESTI4041053//2, TESTI4042711//2, TESTI4046487//1, THYMU2003632//4, THYMU2003760//1, THYMU2005003//2, THYMU2005303//1, THYMU2007658//2, THYMU2009425//3, THYMU2011548//6, THYMU2013386//2, THYMU2014353//2, THYMU2019210//2, THYMU2030068//3, THYMU2032035//2, THYMU2032437//1, THYMU2032655//1, THYMU2033308//1, THYMU2033816//4, THYMU2034314//1,

THYMU2035064//2, THYMU2036085//6, -THYMU2037226//3, THYMU2037233//1, THYMU2037348//2, THYMU2038772//1, THYMU2038797//1, THYMU2040412//1, THYMU2041015//12, THYMU3000028//1, THYMU3000036//2, THYMU3004835//1, THYMU3006168//8, THYMU3006811//2, THYMU3007368//1, TKIDN2002424//2, TKIDN2002632//1, TKIDN2006525//2, TKIDN2009092//1, TKIDN2009889//1,

TKIDN2014771//2, TKIDN2019116//4, TLIVE2000023//5, TLIVE2001828//2, TLIVE2001927//2, TLIVE2002336//1, TLIVE2002690//2, TLIVE2003381//4, TLIVE2004110//1, TOVAR2001281//1, TRACH1000205//6, TRACH2001549//1, TRACH2001684//2, TRACH2006387//6, TRACH2007059//1, TRACH2008300//1, TRACH2020525//4, TRACH2021964//2, TRACH2022553//2, TRACH2025535//1,

TRACH2025911//1, TRACH3000014//1, TRACH3002064//1, TRACH3002650//2, TRACH3004786//4, TRACH3005294//1, TRACH3006149//1, TRACH3007391//1, TRACH3008629//2, TRACH3035199//3, TRACH3036193//1, TSTOM2000442//2, TUTER2000916//1, UTERU2004688//1, UTERU2004929//1, UTERU2006137//1, UTERU2006568//1, UTERU2007444//1, UTERU2020718//2, UTERU2022020//1,

UTERU2025025//1, UTERU2025891//2, UTERU2026090//1, UTERU2026203//3, UTERU2027591//1, UTERU2029953//3, UTERU2031851//2, UTERU2035323//3, UTERU2035469//1, UTERU3000645//4, UTERU3000899//2, UTERU3001240//4, UTERU3001571//2, UTERU3001585//2, UTERU3002209//3, UTERU3002786//1, UTERU3003116//1, UTERU3003776//1, UTERU3006308//3, UTERU3008671//1,

UTERU3009690//1, UTERU3011063//10, UTERU3016789//2

Deduced amino acid sequences of following 70 clones were also detected to have the transmembrane domains by SOSUI.

BLADE2006830//8, BRACE2002589//1, BRACE2011677//2, BRACE2029396//2, BRACE2039823//3, BRACE2039832//1, BRAMY2019111//5, BRAMY2045471//2, BRAWH2008993//1, BRHIP2003272//1, BRHIP2005724//1, BRHIP2008389//3, BRTHA2011321//2, BRTHA2017972//1, BRTHA2018011//2, BRTHA2018443//6, BRTHA3008826//1, CTONG2003348//1, CTONG2015633//2, CTONG2016942//1,

CTONG2019822//9, CTONG2020974//1, FEBRA2000790//1, FEBRA2006519//1, FEBRA2008692//1, FEBRA2028516//2, HCASM2002754//4, HCASM2003099//3, HEART2009680//7, HLUNG2013350//1, HLUNG2015418//3, IMR322013396//2, LIVER2000247//4, NT2RI2009583//8, NT2RI2027157//6, OCBBF2030116//2, PLACE7000502//2, PROST2019487//2, SPLEN2016932//1, SPLEN2037319//2,

SYNOV2001660//1, SYNOV2013637//4, SYNOV4003981//1, SYNOV4005889//1, TBAES2000932//1, TESOP2001796//2, TESOP2006865//1, TESTI2029252//9, TESTI2032643//3, TESTI2050780//6, TESTI4000137//3, TESTI4000155//1, TESTI4006473//1, TESTI4013894//4, TESTI4014801//1, TESTI4032090//2, TESTI4041086//10, THYMU2004284//1, THYMU2030462//1, THYMU2033401//4,

THYMU2034279//1, THYMU2035710//1, THYMU2040925//3, TKIDN2008778//1, TKIDN2012771//4, TRACH3000420//7, UTERU2011220//1, UTERU2021820//2, UTERU2032279//2, UTERU3015069//2

The 664 clones whose deduced amino acid sequences were detected to have functional domains with Pfam are as follows. The search result is indicated as “Clone Name//Functional Domain Name”. When the clone has Multiple Hit Functional Domains, they are listed and demarcated by a double slash mark (//). When the clone has multiple hits of an identical functional domain, each is listed without abridgment.

3NB692002685//R3H domain

3NB692002806//short chain dehydrogenase

3NB692008729//Hr1 repeat motif

ADIPS2000088//Immunoglobulin domain//Immunoglobulin domain//Immunoglobulin domain//Immunoglobulin domain

ASTRO1000009//Delta-aminolevulinic acid dehydratase//FERM domain (Band 4.1 family)

ASTRO2003960//F-box domain.

ASTRO2014923//Ion transport protein

ASTRO3000301//Transmembrane 4 family//Adenovirus E3 region protein CR2

BLADE2005036//Zinc carboxypeptidase

BLADE2007958//WD domain, G-beta repeat//WD domain, G-beta repeat//WD domain, G-beta repeat

BLADE2008539//Carbohydrate phosphorylases

BNGH42003570//EB module//Furin-like cysteine rich region//Thrombospondin type 1 domain

BNGH42007788//WD domain, G-beta repeat//WD domain, G-beta repeat//WD domain, G-beta repeat//WD domain, G-beta repeat//WD domain, G-beta repeat//WD domain, G-beta repeat

BRACE1000258//PH domain//PH domain

BRACE2005457//Sulfate transporter family//Xanthine/uracil permeases family

BRACE2006319//TRAF-type zinc finger//Squash family of serine protease inhibitors//TRAF-type zinc finger

BRACE2008594//Eukaryotic protein kinase domain

BRACE2010489//LysM domain

BRACE2014306//Vesicular monoamine transporter//Ribosomal protein L23//Sugar (and other) transporter//LacY proton/sugar symporter

BRACE2014475//Amidase

BRACE2015314//Bacterial mutT protein

BRACE2016981//Fanconi anaemia group C protein//Bacterial flagellin N-terminus

BRACE2018762//WH1 domain//RanBP1 domain.//Streptomyces extracellular neutral protein//Formyl transferase//K-box region//Tropomyosins

BRACE2026836//Calponin homology (CH) domain

BRACE2027258//Ank repeat//Ank repeat//Ank repeat//Ank repeat//Ank repeat

BRACE2030341//Kinase associated domain 1

BRACE2035381//Lysophospholipase catalytic domain

BRACE2035441//Spectrin repeat//Spectrin repeat//Spectrin repeat

BRACE2038329//TS-N domain//UBA domain

BRACE2041009//TBC domain

BRACE2042550//Thrombospondin type 1 domain//Trypsin Inhibitor like cysteine rich domain//von Willebrand factor type C domain//Thrombospondin type 1 domain

BRACE2043142//Glucose-6-phosphate dehydrogenase

BRACE2044286//CRAL/TRIO domain.//Spectrin repeat

BRACE2045300//Cofilin/tropomyosin-type actin-binding proteins

BRACE2046295//Immunoglobulin domain//EGF-like domain

BRACE2047011//DNA polymerase family B

BRACE3000071//Ank repeat//Ank repeat//Ank repeat

BRACE3000973//Leucine Rich Repeat

BRACE3001002//Lipoprotein

BRACE3001391//Latrophilin/CL-1-like GPS domain//PLAT/LH2 domain//Regulator of G protein signaling domain

BRACE3003192//EGF-like domain//EGF-like domain//EGF-like domain//EGF-like domain//Metallothionein//Keratin, high sulfur B2 protein//EGF-like domain//EGF-like domain//EGF-like domain//EGF-like domain//TB domain//EGF-like domain//EGF-like domain//EGF-like domain//TB domain//EGF-like domain//EGF-like domain

BRACE3004058//FAD/NAD-binding Cytochrome reductase//Oxidoreductase FAD/NAD-binding domain

BRACE3004150//RNA recognition motif. (a.k.a. RRM, RBD, or RNP domain)//RNA recognition motif. (a.k.a. RRM, RBD, or RNP domain)//RNA recognition motif. (a.k.a. RRM, RBD, or RNP domain)

BRACE3004772//SAM domain (Sterile alpha motif)

BRACE3004880//GLTT repeat (12 copies)//GLTT repeat (12 copies)//GLTT repeat (12 copies)//Keratin, high sulfur B2 protein

BRACE3006872//WD domain, G-beta repeat//WD domain, G-beta repeat//WD domain, G-beta repeat

BRACE3007625//Ank repeat//Ank repeat//Ank repeat//Ank repeat//Ank repeat//Ank repeat//Ank repeat//Cytochrome P450

BRACE3008137//PDZ domain (Also known as DHR or GLGF).//PDZ domain (Also known as DHR or GLGF).//PDZ domain (Also known as DHR or GLGF).//PDZ domain (Also known as DHR or GLGF).

BRACE3008384//Rhomboid family

BRACE3008720//GTP1/OBG family//ADP-ribosylation factor family

BRACE3009090//Beige/BEACH domain

BRACE3009708//E1-E2 ATPase//Na+/K+ATPase C-terminus

BRACE3010397//SCP-like extracellular protein

BRACE3011421//Phorbol esters/diacylglycerol binding dom//Diacylglycerol kinase catalytic domain (presumed)//Diacylglycerol kinase accessory domain (presumed)//Ank repeat//Ank repeat

BRACE3013576//Hemagglutinin//SPRY domain//Zinc finger, C3HC4 type (RING finger)

BRACE3014005//Putative integral membrane protein

BRACE3015262//Cytochrome P450

BRACE3015521//EF hand

BRACE3016884//Keratin, high sulfur B2 protein//Flagellar L-ring protein

BRACE3019084//SAM domain (Sterile alpha motif)

BRACE3024073//4Fe-4S iron sulfur cluster binding protein//lactate/malate dehydrogenase//Viral (Superfamily 1) RNA helicase//Ras family

BRACE3025630//Subtilase family

BRACE3027326//LGN motif, putative GEF specific for G-alpha//Rap/ran-GAP

BRACE3027478//Permeases for cytosine/purines, uracil

BRALZ2014484//PH domain

BRALZ2016085//Presenilin//Sugar (and other) transporter//Monocarboxylate transporter

BRAMY2001473//Death domain//ZU5 domain

BRAMY2004771//Leucine Rich Repeat//Leucine rich repeat C-terminal domain//Leucine rich repeat N-terminal domain

BRAMY2005052//Immunoglobulin domain

BRAMY2019300//Leucine Rich Repeat//Leucine rich repeat C-terminal domain//Leucine rich repeat N-terminal domain

BRAMY2021498//Thrombospondin type 1 domain//DnaJ central domain (4 repeats)//Thrombospondin type 1 domain//Thrombospondin type 1 domain//Thrombospondin type 1 domain//Thrombospondin type 1 domain

BRAMY2030109//Phorbol esters/diacylglycerol binding domain (C1 domain)//PHD-finger

BRAMY2031317//PDZ domain (Also known as DHR or GLGF).

BRAMY2036567//SH3 domain

BRAMY2039872//Interferon alpha/beta domain

BRAMY2040592//Transient receptor

BRAMY2042760//PH domain//PH domain

BRAMY2046989//TPR Domain//TPR Domain//TPR Domain//TPR Domain//TPR Domain//TPR Domain

BRAMY2047746//Sodium and potassium ATPases//Ank repeat//Ank repeat//Ank repeat

BRAMY2047751//Regulator of G protein signaling domain//Raf-like Ras-binding domain//Raf-like Ras-binding domain

BRAMY3001794//Geminivirus coat protein//PH domain//Leucine Rich Repeat//Leucine Rich Repeat//Leucine Rich Repeat//Leucine Rich Repeat//Leucine Rich Repeat

BRAMY3002803//P21-Rho-binding domain//Eukaryotic protein kinase domain//Eukaryotic protein kinase domain

BRAMY3004224//Leucine rich repeat N-terminal domain//Leucine Rich Repeat//Leucine Rich Repeat//Leucine Rich Repeat//Leucine Rich Repeat//Leucine Rich Repeat//Leucine rich repeat C-terminal domain

BRAMY3004919//Copper/zinc superoxide dismutase (SODC)//Adenylate and Guanylate cyclase catalytic domain//Adenylate and Guanylate cyclase catalytic domain

BRAMY3005091//Phosphatidylinositol 3- and 4-kinases

BRAMY3005932//Ank repeat

BRAMY3008466//WD domain, G-beta repeat//WD domain, G-beta repeat//WD domain, G-beta repeat//WD domain, G-beta repeat//WD domain, G-beta repeat//WD domain, G-beta repeat

BRAMY4000095//Eukaryotic protein kinase domain

BRAMY4000277//Immunoglobulin domain//Immunoglobulin domain

BRAWH1000127//Plexin repeat//Thrombospondin type 1 domain

BRAWH2001395//Myelin basic protein

BRAWH2001940//NOL1/NOP2/sun family

BRAWH2007658//Immunoglobulin domain

BRAWH2010000//Xylose isomerase

BRAWH2014414//Cadherin domain//Cadherin domain//Cadherin domain//Fructose-bisphosphate aldolase class-//Cadherin domain//Cadherin domain//Cadherin cytoplasmic region

BRAWH2014662//K+ channel tetramerisation domain//ATP synthase Alpha chain, C terminal

BRAWH2014954//PDZ domain (Also known as DHR or GLGF).//PDZ domain (Also known as DHR or GLGF).

BRAWH2016702//AMP-binding enzyme

BRAWH3000078//Adaptin N terminal region//Activin types I and II receptor domain

BRAWH3000314//Fibronectin type III domain

BRAWH3000491//Ribosomal protein S12e

BRAWH3001326//Protein phosphatase 2C

BRAWH3001891//YCF9

BRAWH3002574//Calpain large subunit, domain III//EF hand

BRAWH3002600//Cadherin domain//Cadherin domain//Cadherin domain

BRAWH3002821//C2 domain//C2 domain

BRAWH3003727//ribonuclease.

BRAWH3005912//bZIP transcription factor//bZIP transcription factor//Troponin//TBC domain

BRAWH3008341//Pentaxin family

BRCAN2002562//RNA recognition motif. (a.k.a. RRM, RBD, or RNP domain)

BRCAN2002856//Phosphotyrosine interaction domain (PTB/PID).

BRCAN2002948//Adaptin N terminal region

BRCAN2006063//von Willebrand factor type A domain

BRCAN2009203//SAM domain (Sterile alpha motif)

BRCAN2009432//ADP-ribosylation factor family//Ras family

BRCAN2015464//Gag P30 core shell protein

BRCAN2016619//SH3 domain

BRCAN2017717//Squash family of serine protease inhibitors

BRCAN2021028//Aminopeptidase I zinc metalloprotease (M18)

BRCAN2024451//Raf-like Ras-binding domain//Leptin//Raf-like Ras-binding domain//LGN motif, putative GEF specific for G-alpha GTPase

BRCAN2028355//Eukaryotic protein kinase domain

BRCOC2001505//Myelin basic protein

BRCOC2003213//ATP synthase, Delta/Epsilon chain//tRNA synthetase class II core domain (G, H, P, S and T)

BRCOC2016525//Ank repeat//Ank repeat//Ank repeat//Ank repeat//Ank repeat//Ank repeat//Ank repeat

BRHIP2000819//WD domain, G-beta repeat

BRHIP2000920//Ribosomal protein S9/S16

BRHIP2003786//Ank repeat//Ank repeat//Ank repeat//BTB/POZ domain

BRHIP2004359//Metallo-beta-lactamase superfamily

BRHIP2004814//Phosphate transporter family

BRHIP2005236//Galactose binding lectin domain//Latrophilin Cytoplasmic C-terminal region

BRHIP2005932//PH domain

BRHIP2007616//Sema domain

BRHIP2009414//Uncharacterized protein family

BRHIP2021615//RNA recognition motif. (a.k.a. RRM, RBD, or RNP domain)//RNA recognition motif. (a.k.a. RRM, RBD, or RNP domain)

BRHIP2026288//Prolyl oligopeptidase family//alpha/beta hydrolase fold

BRHIP3000339//Myelin basic protein

BRHIP3008183//Adaptin N terminal region//tRNA (Guanine-1)-methyltransferase

BRHIP3008313//Ank repeat

BRHIP3008405//PH domain

BRHIP3024118//Sodium:galactoside symporter family//Monocarboxylate transporter

BRHIP3025161//Phosphotriesterase family//RhoGEF domain//PH domain//Thaumatin family//GATA zinc finger//FYVE zinc finger//PH domain

BRHIP3027137//Aldehyde dehydrogenase family

BRSSN2000684//Protein-tyrosine phosphatase//Dual specificity phosphatase, catalytic domain

BRSSN2004719//Src homology domain 2

BRSTN2000872//Thioredoxin//Thioredoxin

BRSTN2001067//Rifin/stevor family

BRSTN2004863//Chitin synthase//Glycosyl transferases//Similarity to lectin domain of ricin beta-chain, 3 copies.

BRSTN2004987//tRNA synthetases class I (W and Y)

BRSTN2008418//RhoGAP domain

BRSTN2013741//Ras family

BRTHA2000855//Extracellular link domain

BRTHA2004978//Collagen triple helix repeat (20 copies)

BRTHA2005579//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain

BRTHA2007122//Ank repeat//Ank repeat//Ank repeat//Ank repeat//SAM domain (Sterile alpha motif)

BRTHA2008527//Leucine Rich Repeat//Leucine Rich Repeat//Leucine Rich Repeat//Leucine Rich Repeat//Leucine Rich Repeat

BRTHA2009311//Vertebrate galactoside-binding lectins

BRTHA2010884//Thrombospondin type 1 domain//CUB domain

BRTHA2012980//Cytochrome P450

BRTHA2013262//Keratin, high sulfur B2 protein

BRTHA2014792//SET domain

BRTHA2015406//UBA domain

BRTHA2015878//Gram-negative pili assembly chaperone

BRTHA2016496//Peptidase C13 family

BRTHA2018591//GTPase of unknown function

BRTHA2018624//Galactose binding lectin domain//Activin types I and II receptor domain//Galactose binding lectin domain

BRTHA2018707//RNA recognition motif. (a.k.a. RRM, RBD, or RNP domain)

BRTHA2019048//Domain of unknown function DUF71

BRTHA3002401//Ornithine decarboxylase antizyme

BRTHA3002427//Sulfate transporter family//Sodium:neurotransmitter symporter family

BRTHA3003074//Fanconi anaemia group C protein

BRTHA3003449//Myosin head (motor domain)

BRTHA3005046//Collagen triple helix repeat (20 copies)

BRTHA3008310//Homeobox domain

BRTHA3008778//AMP-binding enzyme

BRTHA3009037//C2 domain//PDZ domain (Also known as DHR or GLGF).//Regulator of G protein signaling domain//Regulator of G protein signaling domain

BRTHA3009090//Cyclic nucleotide-binding domain//Cyclic nucleotide-binding domain//Cyclic nucleotide-binding domain//Glutathione S-transferases.//Uncharacterized protein family UPF0028

BRTHA3013884//Domain associated with PX domains//PX domain//60s Acidic ribosomal protein

BRTHA3015815//AIR synthase related protein

BRTHA3016917//tRNA synthetases class I (C)//tRNA synthetases class I (I, L, M and V)

BRTHA3017589//Immunoglobulin domain//Immunoglobulin domain//Hantavirus glycoprotein G2

BRTHA3017848//Glucose-6-phosphate dehydrogenase//Sugar (and other) transporter

BRTHA3018656//Divalent cation transporter//Divalent cation transporter

COLON2000568//Immunoglobulin domain//Cellulose binding domain//Immunoglobulin domain//Immunoglobulin domain//Immunoglobulin domain//Immunoglobulin domain

COLON2001721//C2 domain

COLON2002520//Myosin head (motor domain)//IQ calmodulin-binding motif

COLON2004478//Immunoglobulin domain//Immunoglobulin domain//Immunoglobulin domain//Immunoglobulin domain//Immunoglobulin domain

CORDB1000140//TRAF-type zinc finger

CORDB2000541//F-actin capping protein, beta subunit

CTONG1000341//EGF-like domain//EGF-like domain//Metallothionein//EGF-like domain//EB module//EGF-like domain//EGF-like domain//EGF-like domain

CTONG1000467//Zinc finger, C3HC4 type (RING finger)

CTONG2000042//Bacterial regulatory proteins, gntR family//Alpha-2-macroglobulin family N-terminal region//Alpha-2-macroglobulin family

CTONG2001877//MutT-like domain

CTONG2004062//E1-E2 ATPase

CTONG2006798//Eukaryotic protein kinase domain//Eukaryotic protein kinase domain

CTONG2008233//DnaJ domain

CTONG2009423//7 transmembrane receptor (rhodopsin family)

CTONG2013178//SEA domain//Trypsin

CTONG2017500//F-box domain.

CTONG2020026//Herpesvirus VP23 like capsid protein

CTONG2024206//Neuregulin family//von Willebrand factor type A domain//EGF-like domain//Response regulator receiver domain//von Willebrand factor type A domain//von Willebrand factor type A domain

CTONG2024749//Alpha-2-macroglobulin family

CTONG2025496//Alpha-2-macroglobulin family N-terminal region//Alpha-2-macroglobulin family

CTONG2028124//AMP-binding enzyme

CTONG2028687//TPR Domain//TPR Domain

CTONG3000084//DNA mismatch repair protein//RhoGEF domain//PH domain//SH3 domain

CTONG3000657//SH3 domain

CTONG3000686//TPR Domain//TPR Domain//TPR Domain//TPR Domain

CTONG3001123//BRCA1 C Terminus (BRCT) domain//BRCA1 C Terminus (BRCT) domain//BRCA1 C Terminus (BRCT) domain//BRCA1 C Terminus (BRCT) domain//BRCA1 C Terminus (BRCT) domain

CTONG3001370//Alpha-2-macroglobulin family N-terminal region//Alpha-2-macroglobulin family

CTONG3002127//C2 domain//C2 domain

CTONG3002674//Calponin homology (CH) domain

CTONG3003737//Leucine rich repeat N-terminal domain//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine rich repeat C-terminal domain//Fusion glycoprotein F0.

CTONG3003972//RNA recognition motif. (a.k.a. RRM, RBD, or RNP domain)

CTONG3004072//Beta type Zein//Keratin, high sulfur B2 protein

CTONG3005325//TS-N domain//UBA domain//Transposase

CTONG3005648//Putative undecaprenyl diphosphate synthase

CTONG3006067//DnaJ central domain (4 repeats)

CTONG3006186//PDZ domain (Also known as DHR or GLGF)//Apolipoprotein A1/A4/E family//WW domain

CTONG3008831//RNA recognition motif. (a.k.a. RRM, RBD, or RNP domain)

CTONG3009028//Helicases conserved C-terminal domain

CTONG3009385//TPR Domain//TPR Domain//TPR Domain//TPR Domain//TPR Domain//TPR Domain//TPR Domain//TPR Domain//TPR Domain//TPR Domain

D3OST2002182//Glycosyl transferase family 8

D3OST2002648//7 transmembrane receptor (rhodopsin family)//7 transmembrane receptor (rhodopsin family)

D3OST3000169//SH3 domain//SAM domain (Sterile alpha motif)

DFNES2000146//Plexin repeat//Thrombospondin type 1 domain

DFNES2001108//PH domain

DFNES2005266//Thrombospondin type 1 domain

DFNES2011499//WD domain, G-beta repeat

FCBBF3004502//Terpene synthase family//YCF9

FCBBF3007540//RhoGEF domain//PH domain

FCBBF3009888//Keratin, high sulfur B2 protein//u-PAR/Ly-6 domain

FCBBF3012170//Thrombospondin type 1 domain

FCBBF3012288//Fibronectin type III domain

FCBBF3013307//DEAD/DEAH box helicase//Helicases conserved C-terminal domain

FEBRA2000253//Flagellar L-ring protein

FEBRA2007708//Fusion glycoprotein F0.//Xanthine/uracil permeases family//Sulfate transporter family

FEBRA2007801//IBR domain

FEBRA2008311//7 transmembrane receptor (rhodopsin family)//7 transmembrane receptor (rhodopsin family)

FEBRA2008468//alpha/beta hydrolase fold

FEBRA2021571//von Willebrand factor type D domain

FEBRA2024150//DENN (AEX-3) domain

FEBRA2026984//tRNA synthetases class I (W and Y)//Putative tRNA binding domain

HCASM2001301//Eukaryotic protein kinase domain

HCASM2002918//ATP synthase Alpha chain, C terminal

HCHON2000028//RhoGAP domain

HCHON2001084//FecCD transport family//Sugar (and other) transporter

HCHON2001217//Cullin family

HCHON2001577//Collagen triple helix repeat (20 copies)//Heavy-metal-associated domain

HCHON2001712//Sodium:dicarboxylate symporter family

HCHON2002676//Glycosyl hydrolases family 39

HCHON2004007//E1-E2 ATPase

HCHON2004531//Ubiquitin family//UBA domain//Integrins, beta chain//UBA domain

HCHON2004776//Protein of unknown function DUF93

HCHON2005921//PMP-22/EMP/MP20/Claudin family

HCHON2006250//WD domain, G-beta repeat//WD domain, G-beta repeat//WD domain, G-beta repeat//WD domain, G-beta repeat

HEART1000139//Troponin

HEART2001680//Immunoglobulin domain//Immunoglobulin domain//Immunoglobulin domain//Immunoglobulin domain//Immunoglobulin domain

HEART2001756//Bacterial transcriptional regulator

HEART2006131//Thiamine pyrophosphate enzymes

HEART2006909//CBS domain//CBS domain

HEART2010495//Tau and MAP proteins, tubulin-binding

HHDPC1000118//Adenylate kinase//Shikimate kinase//Deoxynucleoside kinase//Pyridoxal-phosphate dependent enzyme

HLUNG1000017//Reprolysin family propeptide

HLUNG2000014//Lectin C-type domain

HLUNG2001996//SH3 domain

HLUNG2002465//PH domain//RhoGEF domain//SH3 domain

HLUNG2002958//EF hand

HLUNG2011298//Oxidoreductase FAD/NAD-binding domain

HLUNG2013851//Pumilio-family RNA binding domains (aka PUM-HD, Pumilio homology domain)//Pumilio-family RNA binding domains (aka PUM-HD, Pumilio homology domain)

HLUNG2014262//von Willebrand factor type A domain//von Willebrand factor type A domain

HLUNG2017350//Connexin

HSYRA2005456//Fibronectin type III domain

HSYRA2005496//emp24/gp25L/p24 family

HSYRA2009075//Fibronectin type III domain

HSYRA2009102//Integral membrane protein DUF6

IMR322000127//Zinc finger, C2H2 type

IMR322000917//Zinc finger, C2H2 type

IMR322006495//Tropomyosins

KIDNE1000064//Integral membrane protein DUF7//Sugar (and other) transporter//Transmembrane 4 family//Zn-finger in Ran binding protein and others.

KIDNE2000832//Amino acid permease//Transmembrane amino acid transporter protein//Sodium/hydrogen exchanger family

KIDNE2000846//Sodium:neurotransmitter symporter family

KIDNE2001361//Domain of unknown function DUF19

KIDNE2001847//RhoGAP domain//SH3 domain

KIDNE2006580//Cytochrome P450

KIDNE2011635//Sodium:solute symporter family

KIDNE2012945//CUB domain//Pentaxin family

LYMPB2000083//Class I Histocompatibility antigen, domains alpha 1 and 2//Class I Histocompatibility antigen, domains alpha 1 and 2//Immunoglobulin domain

MESAN2006563//PH domain

MESAN2012054//PQQ enzyme repeat//PQQ enzyme repeat

NESOP2001433//Immunoglobulin domain//Immunoglobulin domain//Immunoglobulin domain//Immunoglobulin domain//Immunoglobulin domain

NESOP2001656//Polyomavirus coat protein

NHNPC2001816//Regulator of G protein signaling domain

NOVAR2000136//Thioredoxin//CTF/NF-I family//Calsequestrin

NOVAR2001108//Immunoglobulin domain//Immunoglobulin domain//Immunoglobulin domain//Immunoglobulin domain

NT2NE2003252//Eukaryotic protein kinase domain

NT2NE2006531//KRAB box//PHD-finger//Transcription factor S-II (TFIIS)//Zinc finger, C2H2 type

NT2NE2006909//Influenza Matrix protein (M1)//metallopeptidase family M24

NT2RI2004618//Cytosolic long-chain acyl-CoA thioestease

NT2RI2005166//F-box domain.//WD domain, G-beta repeat

NT2RI2008724//GGL domain

NT2RI2025909//Mitochondrial carrier proteins//Mitochondrial carrier proteins//Mitochondrial carrier proteins

NT2RI2025957//PDZ domain (Also known as DHR or GLGF).

NT2RI3000622//TBC domain

NT2RI3002842//Hsp20/alpha crystallin family

NT2RI3003382//Rotavirus RNA-binding Protein 53 (NS53)

NT2RI3004510//Pyridine nucleotide-disulphide oxidoreductase//FAD binding domain//Flavin containing amine oxidase//Phytoene dehydrogenase related enzyme

NT2RI3006171//Immunoglobulin domain//Immunoglobulin domain//Immunoglobulin domain//Immunoglobulin domain

NT2RI3006340//Immunoglobulin domain//Immunoglobulin domain//Fibronectin type III domain//Fibronectin type III domain//Fibronectin type III domain//Fibronectin type III domain//Fibronectin type III domain//Immunoglobulin domain//Ribosomal protein S14p/S29e //Immunoglobulin domain//Immunoglobulin domain

NT2RI3006376//DENN (AEX-3) domain//PLAT/LH2 domain

NT2RI3006673//Fibronectin type III domain//Fibronectin type III domain//Fibronectin type III domain//Protein-tyrosine phosphatase//Dual specificity phosphatase, catalytic domain//Protein-tyrosine phosphatase

NT2RI3007158//FYVE zinc finger

NT2RI3007291//Collagen triple helix repeat (20 copies)

NT2RI3007543//DnaJ domain

NT2RI3007978//Glutamine amidotransferase class-I

NT2RI3008652//FERM domain (Band 4.1 family)//Uncharacterised protein family UPF0058//Biopterin-dependent aromatic amino acid hydroxylase

NT2RP7000359//FERM domain (Band 4.1 family)//Insulin-like growth factor binding proteins//PDZ domain (Also known as DHR or GLGF).

NT2RP7000466//CUB domain//CXXC zinc finger//EGF-like domain//Granulins//Keratin, high sulfur B2 protein//Trypsin Inhibitor like cysteine rich domain

NT2RP7004027//CUB domain//Sushi domain (SCR repeat)

NT2RP7004123//Hepatitis delta virus delta antigen

NT2RP7005118//GTPase-activator protein for Ras-like GTPase//IQ calmodulin-binding motif//WW domain

NT2RP7005529//PH domain//RhoGEF domain

NT2RP7009147//WD domain, G-beta repeat//WD domain, G-beta repeat//WD domain, G-beta repeat

NT2RP7010599//Lipase

NT2RP7011570//Gag P30 core shell protein

NT2RP7013795//WD domain, G-beta repeat//WD domain, G-beta repeat

NT2RP7014005//Glutamine amidotransferase class-I

NT2RP7017474//Phosphoglucose isomerase

NT2RP8000296//BTB/POZ domain//Kelch motif//Kelch motif//Kelch motif//Kelch motif//Kelch motif//Kelch motif

NT2RP8000483//RhoGAP domain

NTONG2000413//Astacin (Peptidase family M12A)//Hemopexin//Matrixin

NTONG2003852//Phosphotyrosine interaction domain (PTB/PID).

NTONG2005277//Ank repeat//Ank repeat//Ank repeat//Ank repeat//Ank repeat//Ank repeat//Ank repeat//Ank repeat

NTONG2006354//Ank repeat

NTONG2007517//BTB/POZ domain

OCBBF2004826//PH domain//Raf-like Ras-binding domain//Transaldolase//PDZ domain (Also known as DHR or GLGF).//RhoGEF domain

OCBBF2004883//WD domain, G-beta repeat//WD domain, G-beta repeat//WD domain, G-beta repeat//WD domain, G-beta repeat//WD domain, G-beta repeat

OCBBF2006058//Acyl-CoA dehydrogenase

OCBBF2006764//Sushi domain (SCR repeat)//CUB domain//Sushi domain (SCR repeat)//CUB domain//Sushi domain (SCR repeat)

OCBBF2007028//SH3 domain

OCBBF2007068//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ank repeat//Ribosomal protein L34//Ank repeat//Ank repeat//Ank repeat//Ank repeat

OCBBF2008770//TBC domain

OCBBF2010140//Alphavirus E1 glycoprotein

OCBBF2010416//Major intrinsic protein

OCBBF2019823//lactate/malate dehydrogenase

OCBBF2020838//Fork head domain

OCBBF2021323//Regulatory subunit of type II PKA R-subunit

OCBBF2022351//WD domain, G-beta repeat//WD domain, G-beta repeat//WD domain, G-beta repeat//WD domain, G-beta repeat//WD domain, G-beta repeat//WD domain, G-beta repeat

OCBBF2025527//NAD-dependent glycerol-3-phosphate dehydrogenase

OCBBF2031167//Reprolysin family propeptide//Pancreatic hormone peptides//Reprolysin (M12B) family zinc metalloprotease//Disintegrin//Beta defensin//Radical activating enzymes//EB module//EGF-like domain//Delta serrate ligand

OCBBF2033869//CUB domain

OCBBF2035110//PLAT/LH2 domain

OCBBF2036743//KRAB box//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//PHD-finger//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type

OCBBF2037340//Alpha crystallin A chain, N terminal//Archaeal ATPase//Dual specificity phosphatase, catalytic domain

OCBBF2037547//PH domain//Raf-like Ras-binding domain//Transaldolase//PDZ domain (Also known as DHR or GLGF).//RhoGEF domain//PH domain

OCBBF2037598//Immunoglobulin domain//Immunoglobulin domain//Immunoglobulin domain//Fibronectin type III domain//Fibronectin type III domain

OCBBF2037638//Pyridine nucleotide-disulphide oxidoreductase//Pyridine nucleotide-disulphide oxidoreductase

OCBBF2038317//Syndecan domain//BNR repeat//BNR repeat//BNR repeat//BNR repeat//BNR repeat//PKD domain

OCBBF3009279//KH domain//Zinc finger, C3HC4 type (RING finger)

PEBLM2002594//ABC transporter//Aldehyde oxidase and xanthine dehydrogenase, C terminus

PEBLM2004666//WD domain, G-beta repeat//Gram-negative pili assembly chaperone//WD domain, G-beta repeat//WD domain, G-beta repeat//WD domain, G-beta repeat//WD domain, G-beta repeat//WD domain, G-beta repeat

PERIC1000147//Syndecan domain

PERIC2001228//Leucine Rich Repeat//Leucine Rich Repeat//Leucine Rich Repeat//Leucine Rich Repeat//Leucine Rich Repeat//Leucine Rich Repeat//Leucine Rich Repeat

PERIC2003720//Ezrin/radixin/moesin family

PERIC2009086//7 transmembrane receptor (rhodopsin family)//Peroxidase

PLACE5000171//Chitin binding Peritrophin-A domain//HYR domain//Plant PEC family metallothionein//Sushi domain (SCR repeat)//von Willebrand factor type A domain

PLACE5000282//Collagen triple helix repeat (20 copies)//Heavy-metal-associated domain

PLACE6012574//ENV polyprotein (coat polyprotein)

PLACE6019385//REV protein (anti-repression trans-activator protein)

PLACE6020031//Ank repeat//Ank repeat

PLACE7000514//Filamin/ABP280 repeat.

PLACE7002641//LPP20 lipoprotein precursor//HRDC domain//Dihydrodipicolinate synthetase family

PLACE7006051//ENV polyprotein (coat polyprotein)

PLACE7008431//Phosphatidylinositol-4-phosphate 5-Kinase

PROST1000184//7 transmembrane receptor (Secretin family)

PROST2008993//BRCA1 C Terminus (BRCT) domain

PROST2016462//WW domain//PH domain//RhoGAP domain

PROST2017367//Transglutaminase family

PROST2018090//Sushi domain (SCR repeat)//Sushi domain (SCR repeat)//Chitin binding Peritrophin-A domain//HYR domain//Sushi domain (SCR repeat)

PROST2018511//Ras association (RalGDS/AF-6) domain//PH domain//Src homology domain 2

PUAEN2002616//Src homology domain 2

PUAEN2005930//Extracellular link domain//PH domain

PUAEN2006328//TBC domain

PUAEN2007044//TruB family pseudouridylate synthase (N terminal domain)

PUAEN2009174//L1 (late) protein//Alpha-2-macroglobulin family

PUAEN2009795//Ribosomal protein S3, C-terminal domain//EGF-like domain//C1q domain

PUAEN2009852//Eukaryotic protein kinase domain

RECTM2000433//Jacalin-like lectin domain

SKMUS2006394//Ank repeat//Ank repeat//Ank repeat//Ank repeat//Ank repeat//Ank repeat//Ank repeat

SMINT1000192//Small hydrophilic plant seed proteins

SMINT2002743//ENV polyprotein (coat polyprotein)

SMINT2010076//Immunoglobulin domain//Immunoglobulin domain//Immunoglobulin domain//Immunoglobulin domain//Immunoglobulin domain

SMINT2011888//Immunoglobulin domain//Cellulose binding domain//Immunoglobulin domain//Immunoglobulin domain//Immunoglobulin domain//Immunoglobulin domain

SMINT2015787//Serum amyloid A protein//Immunoglobulin domain

SPLEN2001599//Immunoglobulin domain

SPLEN2002147//Phosphatidylinositol transfer protein

SPLEN2002467//DB module//F-box domain.//Leucine Rich Repeat

SPLEN2006122//RNA recognition motif. (a.k.a. RRM, RBD, or RNP domain)//RNA recognition motif. (a.k.a. RRM, RBD, or RNP domain)

SPLEN2010912//DEAD/DEAH box helicase//Helicases conserved C-terminal domain

SPLEN2012624//Ank repeat//Ank repeat//Ank repeat//Sodium:neurotransmitter symporter family

SPLEN2015267//Immunoglobulin domain//Immunoglobulin domain//Immunoglobulin domain//Immunoglobulin domain

SPLEN2015679//ATP synthase delta (OSCP) subunit

SPLEN2021701//Class I Histocompatibility antigen, domains alpha 1 and 2//Class I Histocompatibility antigen, domains alpha 1 and 2//Immunoglobulin domain

SPLEN2030335//AMP-binding enzyme

SPLEN2031547//Integral membrane protein//Integral membrane protein

SPLEN2031780//Domain of unknown function DUF139//Domain of unknown function DUF139

SPLEN2033098//TNFR/NGFR cysteine-rich region

SPLEN2034081//Insulin-like growth factor binding proteins

SPLEN2036326//GPR1/FUN34/yaaH family//PMP-22/EMP/MP20/Claudin family

SPLEN2036821//Mitochondrial carrier proteins

SPLEN2037722//Immunoglobulin domain//Immunoglobulin domain

STOMA2004294//Immunoglobulin domain

SYNOV2005448//Apidaecin

SYNOV2005817//Domain of unknown function DUF19//Tissue factor

SYNOV2006430//Nitrogen regulatory protein P-II

SYNOV2014400//EGF-like domain//Granulins//Granulins//EGF-like domain

SYNOV2021320//Src homology domain 2

SYNOV3000231//Immunoglobulin domain//Immunoglobulin domain//Immunoglobulin domain//Immunoglobulin domain

SYNOV3000302//Immunoglobulin domain//Immunoglobulin domain//Immunoglobulin domain//Immunoglobulin domain

SYNOV4002392//lactate/malate dehydrogenase

SYNOV4002883//Adenosylmethionine decarboxylase

SYNOV4007521//Immunoglobulin domain//Immunoglobulin domain

SYNOV4007553//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine rich repeat C-terminal domain//TIR domain

SYNOV4007671//Syntaxin//Fusion glycoprotein F0.

SYNOV4008440//Adaptin N terminal region

TBAES2001171//NOL1/NOP2/sun family

TBAES2001229//Ribosomal protein L23

TBAES2003550//Glucose-6-phosphate dehydrogenase

TBAES2004055//Ribosomal protein S11

TESOP2000801//Eukaryotic protein kinase domain

TESOP2001166//Src homology domain 2

TESOP2001953//Leucine Rich Repeat//Leucine Rich Repeat//Leucine Rich Repeat//Leucine Rich Repeat//Leucine Rich Repeat//Leucine Rich Repeat//Leucine Rich Repeat

TESOP2004114//Lysyl hydrolase//Lysyl hydrolase

TESOP2005485//Immunoglobulin domain//Immunoglobulin domain

TESOP2009121//DNA polymerase (viral) C-terminal domain

TESTI1000257//GntP family permease//Sugar (and other) transporter

TESTI1000390//Bromodomain//Atrial natriuretic peptide

TESTI1000545//TPR Domain//TPR Domain//TPR Domain//LPP20 lipoprotein precursor//HRDC domain//Adaptin N terminal region//Dihydrodipicolinate synthetase family

TESTI2000443//Leucine Rich Repeat//Leucine Rich Repeat//Leucine Rich Repeat

TESTI2000644//Small cytokines (intecrine/chemokine), interleukin-8 like

TESTI2002036//Ion transport protein//Transmembrane region cyclic Nucleotide Gated Channel

TESTI2002618//Reprolysin (M12B) family zinc metalloprotease//Reprolysin family propeptide

TESTI2002928//Syndecan domain

TESTI2003347//Connexin//Cytochrome b559, alpha (gene psbE) and beta (gene psbF) subunits.

TESTI2004700//Leucine Rich Repeat//Leucine Rich Repeat//Leucine Rich Repeat//Leucine Rich Repeat

TESTI2005610//CD36 family

TESTI2006648//Ion transport protein//ABC transporter transmembrane region.//PEP-utilizing enzymes//Phosphoribulokinase//Elongation factor Tu family

TESTI2014716//RNA recognition motif. (a.k.a. RRM, RBD, or RNP domain)//RNA recognition motif. (a.k.a. RRM, RBD, or RNP domain)//RNA recognition motif. (a.k.a. RRM, RBD, or RNP domain)

TESTI2024567//7 transmembrane receptor (metabotropic glutamate family)

TESTI2026505//Domain of unknown function DUF123//FYVE zinc finger//PH domain//RhoGEF domain

TESTI2027019//Leucine Rich Repeat

TESTI2034520//ABC transporter

TESTI2034767//Collagen triple helix repeat (20 copies)//Collagen triple helix repeat (20 copies)//Collagen triple helix repeat (20 copies)//Collagen triple helix repeat (20 copies)//Collagen triple helix repeat (20 copies)//Collagen triple helix repeat (20 copies)//Collagen triple helix repeat (20 copies)

TESTI2040018//Hepatitis C virus RNA dependent RNA polymerase

TESTI2044796//Zinc finger, C3HC4 type (RING finger)

TESTI2049469//Chitinases class I

TESTI2050137//Phosphotyrosine interaction domain (PTB/PID)//Src homology domain 2

TESTI2050987//Zinc finger, C3HC4 type (RING finger)//SPRY domain

TESTI2051867//Ribosomal protein L4/L1 family

TESTI2052693//Src homology domain 2

TESTI2053621//EF hand//EF hand//Glutathione peroxidases//EF hand

TESTI4000014//PPR repeat//PPR repeat//PPR repeat//PPR repeat//PPR repeat//ENTH domain//PPR repeat//PPR repeat//PPR repeat//Ribosomal protein L22p/L17e//Interleukin 10//PPR repeat

TESTI4000079//Phosphopantetheine attachment site//PH domain

TESTI4000288//Dynamin GTPase effector domain

TESTI4000349//HECT-domain (ubiquitin-transferase).

TESTI4000462//Keratin, high sulfur B2 protein

TESTI4000724//Vesicular monoamine transporter//Sugar (and other) transporter//Monocarboxylate transporter

TESTI4000970//Ezrin/radixin/moesin family

TESTI4001148//Enol-ase//ATP synthase delta (OSCP) subunit

TESTI4001527//UDP-glucoronosyl and UDP-glucosyl transferase

TESTI4001561//Acyltransferase

TESTI4002491//NSF attachment protein

TESTI4002552//E1-E2 ATPase//Na+/K+ ATPase C-terminus

TESTI4006326//von Willebrand factor type A domain

TESTI4006546//Tudor domain//Tudor domain//Tudor domain

TESTI4006819//Enol-ase

TESTI4007064//DENN (AEX-3) domain//PPR repeat//LIM domain containing proteins

TESTI4007163//Sodium:neurotransmitter symporter family

TESTI4007382//Nickel-dependent hydrogenases

TESTI4007778//Calponin homology (CH) domain//Calponin homology (CH) domain//Spectrin repeat//Spectrin repeat//Spectrin repeat//Spectrin repeat//EF hand//EF hand

TESTI4007810//DNA ligase

TESTI4008429//E1-E2 ATPase//Domain of unknown function

TESTI4009160//Kinesin motor domain//Kinesin motor domain

TESTI4009374//RNA recognition motif. (a.k.a. RRM, RBD, or RNP domain)//Ham1 family//RNA recognition motif. (a.k.a. RRM, RBD, or RNP domain)//RNA recognition motif. (a.k.a. RRM, RBD, or RNP domain)//Double-stranded RNA binding motif

TESTI4009608//emp24/gp25L/p24 family

TESTI4009881//Bacterial type II secretion system protein

TESTI4010713//C2 domain

TESTI4010831//WD domain, G-beta repeat

TESTI4010851//Ubiquitin carboxyl-terminal hydrolases//Ubiquitin carboxyl-terminal hydrolase f//eIF4-gamma/eIF5/eIF2-epsilon//Rifin/stevor family

TESTI4011484//SAM domain (Sterile alpha motif)

TESTI4011745//Bromodomain

TESTI4011956//PH domain

TESTI4012406//Kringle domain

TESTI4012448//Matrixin//Hemopexin//Hemopexin//Hemopexin//Hemopexin

TESTI4012679//DNA photolyase

TESTI4013369//ATP synthase subunit C

TESTI4014924//Floricaula/Leafy protein

TESTI4015471//Tropomyosins

TESTI4016110//DnaJ domain

TESTI4016882//SH3 domain//SH3 domain

TESTI4016925//Aminotransferases class-III//Pyridoxal-phosphate dependent enzyme

TESTI4017001//bZIP transcription factor

TESTI4017137//Keratin, high sulfur B2 protein

TESTI4017575//MSP (Major sperm protein) domain

TESTI4018152//FERM domain (Band 4.1 family)

TESTI4018555//Granulins

TESTI4018835//E1-E2 ATPase//E1-E2 ATPase

TESTI4018886//Fibronectin type III domain//Fibronectin type III domain//Fibronectin type III domain

TESTI4019140//GATA zinc finger

TESTI4019566//Helicases conserved C-terminal domain//Tudor domain

TESTI4019843//SH3 domain//RhoGEF domain//PH domain

TESTI4020092//Laminin G domain

TESTI4020920//D-isomer specific 2-hydroxyacid dehydrogenase, catalytic domain

TESTI4021294//Cyclin//Immunoglobulin domain

TESTI4021478//E1-E2 ATPase//E1-E2 ATPase//E1-E2 ATPase

TESTI4022716//DEAD/DEAH box helicase//Helicases conserved C-terminal domain

TESTI4023555//Lectin C-type domain

TESTI4025920//Adaptin N terminal region

TESTI4026192//Domain of unknown function

TESTI4026510//DEAD/DEAH box helicase//Helicases conserved C-terminal domain

TESTI4027557//Vertebrate galactoside-binding lectins//Vertebrate galactoside-binding lectins

TESTI4028059//Phosphofructokinase//Phosphofructokinase

TESTI4028429//WAP-type (Whey Acidic Protein) ‘four-disulfide core’

TESTI4028612//Major intrinsic protein

TESTI4028880//Sugar (and other) transporter//Sodium:galactoside symporter family

TESTI4028983//Serum amyloid A protein TESTI4029836//E1-E2 ATPase//E1-E2 ATPase//Neuraxin and MAP1B proteins//E1-E2 ATPase//Cof family

TESTI4030505//Metallothionein family 5

TESTI4030603//Collagen triple helix repeat (20 copies)

TESTI4032895//ATP synthase, Delta/Epsilon chain//Tropomyosins//Protein of unknown function

TESTI4034432//Peptidyl-tRNA hydrolase domain

TESTI4034632//Ribosomal protein S3, C-terminal domain//Similarity to lectin domain of ricin beta-chain, 3 copies

TESTI4034912//Adhesin lipoprotein//Vesiculovirus phosphoprotein

TESTI4035063//Myosin tail//CAP-Gly domain

TESTI4035498//Cell division protein

TESTI4036909//Viral (Superfamily 1) RNA helicase//Heavy-metal-associated domain//Viral (Superfamily 1) RNA helicase

TESTI4038492//Serum amyloid A protein

TESTI4039659//DnaJ domain

TESTI4041053//Ank repeat//Ank repeat//Ank repeat//Ank repeat//Armadillo/beta-catenin-like repeats//Armadillo/beta-catenin-like repeats//Armadillo/beta-catenin-like repeats//Armadillo/beta-catenin-like repeats//Armadillo/beta-catenin-like repeats//Armadillo/beta-catenin-like repeats

TESTI4044084//Domain of unknown function

TESTI4046487//Hantavirus nucleocapsid protein

TESTI4046819//Metallothionein//PTS HPr component phosphorylation sites

THYMU1000496//Kinesin motor domain

THYMU2004693//CX module

THYMU2005303//Immunoglobulin domain

THYMU2006420//NAD(P) transhydrogenase beta subunit

THYMU2008725//Similarity to lectin domain of ricin beta-chain, 3 copies.//Fibronectin type III domain//Fibronectin type III domain//Fibronectin type III domain//Fibronectin type III domain

THYMU2009425//7 transmembrane receptor (rhodopsin family)

THYMU2011548//7 transmembrane receptor (rhodopsin family)

THYMU2011736//EGF-like domain//EGF-like domain//EB module//EGF-like domain//TB domain//EGF-like domain//EGF-like domain

THYMU2016204//Metallothionein

THYMU2019210//Class I Histocompatibility antigen, domains alpha 1 and 2//Class I Histocompatibility antigen, domains alpha 1 and 2//Immunoglobulin domain

THYMU2023711//Immunoglobulin domain//Immunoglobulin domain//Immunoglobulin domain//Immunoglobulin domain

THYMU2027695//Immunoglobulin domain//Immunoglobulin domain//Immunoglobulin domain//Immunoglobulin domain

THYMU2027734//Parvovirus coat protein VP2

THYMU2032014//SH3 domain

THYMU2033079//ABC transporter

THYMU2035319//RNA recognition motif. (a.k.a. RRM, RBD, or RNP domain)//RNA recognition motif. (a.k.a. RRM, RBD, or RNP domain)//RNA recognition motif. (a.k.a. RRM, RBD, or RNP domain)

THYMU2035735//FHA domain//SNAP-25 family//Borrelia ORF-A

THYMU2036459//GTP1/OBG family

THYMU2037226//SH3 domain//TPR Domain//TPR Domain//TPR Domain//TPR Domain

THYMU2038369//Regulatory subunit of type II PKA R-subunit

THYMU2038615//PH domain

THYMU2038797//Lectin C-type domain

THYMU2041015//Sodium:galactoside symporter family//LacY proton/sugar symporter//Domain of unknown function//Monocarboxylate transporter//Polysaccharide biosynthesis protein//Sugar (and other) transporter

THYMU3000028//Zona pellucida-like domain

THYMU3000133//Viral (Superfamily 1) RNA helicase

THYMU3001234//PH domain

THYMU3001379//3′5′-cyclic nucleotide phosphodiesterase//Elongation factor Tu family

THYMU3003212//Cytidine and deoxycytidylate deaminase zinc-binding region

THYMU3003763//Leucine rich repeat N-terminal domain//Polyomavirus coat protein

THYMU3004835//Galactosyltransferase

THYMU3006172//C2 domain//C2 domain

THYMU3007137//PDZ domain (Also known as DHR or GLGF).//PDZ domain (Also known as DHR or GLGF).

THYMU3008171//TPR Domain

THYMU3008436//Phosphofructokinase//Phosphofructokinase

TLIVE2000023//Integral membrane protein

TLIVE2002336//Metalloenzyme superfamily//Sulfatase//Type I phosphodiesterase/nucleotide pyrophosphatase

TLIVE2002338//Transforming growth factor beta like domain

TLIVE2002690//von Willebrand factor type D domain

TLIVE2003225//CUB domain//Sushi domain (SCR repeat)//CUB domain//Sushi domain (SCR repeat)

TLIVE2003381//7 transmembrane receptor (metabotropic glutamate family)

TLIVE2007132//Syndecan domain

TLIVE2008229//TPR Domain//TPR Domain

TLIVE2009541//TBC domain

TRACH2001443//TIR domain

TRACH2001549//Cyclic nucleotide-binding domain

TRACH2005811//Kinesin motor domain

TRACH2006387//NADH-ubiquinone oxidoreductase chain 4//7 transmembrane receptor (rhodopsin family)

TRACH2007059//DnaJ domain//Integrins, beta chain//PA domain

TRACH2009310//Armadillo/beta-catenin-like repeats//Eukaryotic protein kinase domain//RIO1/ZK632.3/MJ0444 family

TRACH2019473//Iron/manganese superoxide dismutases (SODM)

TRACH2021398//RhoGAP domain

TRACH2022425//Immunoglobulin domain//Subtilase family//Immunoglobulin domain//Immunoglobulin domain//Immunoglobulin domain//Immunoglobulin domain

TRACH2022553//Immunoglobulin domain//Immunoglobulin domain//Immunoglobulin domain//Immunoglobulin domain

TRACH2022649//Immunoglobulin domain//Immunoglobulin domain//Immunoglobulin domain//Immunoglobulin domain

TRACH2023299//Squalene and phytoene synthases//PH domain//tRNA synthetases class I (E and Q)

TRACH2025535//PH domain

TRACH2025749//Zinc finger, C3HC4 type (RING finger)

TRACH3001427//UBX domain

TRACH3002168//Immunoglobulin domain//Immunoglobulin domain//Immunoglobulin domain

TRACH3003379//Protein phosphatase 2A regulatory B subunit

TRACH3004786//PMP-22/EMP/MP20/Claudin family

TRACH3004840//Rop protein

TRACH3005479//Glycosyl transferases group 1

TRACH3005549//Immunoglobulin domain//Immunoglobulin domain

TRACH3006470//Glycosyl transferases group 1

TRACH3007479//WW domain//HECT-domain (ubiquitin-transferase).

TRACH3008093//Putative undecaprenyl diphosphate synthase

TRACH3008629//Cadherin domain//Cadherin domain//Cadherin domain//Cadherin domain//Cadherin domain//PQQ enzyme repeat

TRACH3008713//NSF attachment protein

TRACH3009455//Src homology domain 2//FERM domain (Band 4.1 family)//Src homology domain 2

TRACH3034731//Ras association (RalGDS/AF-6) domain

TRACH3035235//S-100/ICaBP type calcium binding domain

TRACH3035526//Immunoglobulin domain//Cellulose binding domain//Immunoglobulin domain//Immunoglobulin domain//Immunoglobulin domain//Immunoglobulin domain

TRACH3036193//picornavirus capsid protein//Thaumatin family//Picornavirus core protein 2A//Picornavirus 2B protein//Extracellular link domain//RNA helicase//3C cysteine protease (picornain 3C)//RNA dependent RNA polymerase

TRACH3036609//Immunoglobulin domain

TSTOM2000442//Immunoglobulin domain//Immunoglobulin domain//Immunoglobulin domain//Immunoglobulin domain

TSTOM2000553//C2 domain

TUTER2000425//KRAB box

UTERU1000024//NOL1/NOP2/sun family//NOL1/NOP2/sun family

UTERU1000031//ENTH domain//VHS domain

UTERU1000337//Protein phosphatase 2C

UTERU2005621//Protein-tyrosine phosphatase//Dual specificity phosphatase, catalytic domain

UTERU2006115//Adaptin N terminal region

UTERU2006568//IBR domain

UTERU2007724//Calponin homology (CH) domain//Calponin family//Calponin family//Calponin family

UTERU2017762//Ubiquitin family

UTERU2019706//TCP-1/cpn60 chaperonin family//TCP-1/cpn60 chaperonin family

UTERU2019940//Ribosomal protein L30p/L7e

UTERU2025025//Eukaryotic protein kinase domain

UTERU2026025//RNA recognition motif. (a.k.a. RRM, RBD, or RNP domain)

UTERU2026090//Lectin (probable mannose binding)

UTERU2033375//Ubiquitin carboxyl-terminal hydrolase family 2

UTERU2035328//WW domain//WW domain//WW domain//FF domain//FF domain//FF domain

UTERU2035331//Fibrillar collagen C-terminal domain

UTERU2035452//EGF-like domain//Metallothionein//EGF-like domain

UTERU2035745//Myosin head (motor domain)//Aldehyde oxidase and xanthine dehydrogenase, C terminus

UTERU2036089//RhoGAP domain

UTERU2038251//PH domain

UTERU3000645//PMP-22/EMP/MP20/Claudin family

UTERU3000828//315′-cyclic nucleotide phosphodiesterase//Elongation factor Tu family//Elongation factor G C-terminus

UTERU3001240//Copper/zinc superoxide dismutase (SODC)//Adenylate and Guanylate cyclase catalytic domain

UTERU3001585//Cytochrome P450

UTERU3001652//Wiskott Aldrich syndrome homology region 2

UTERU3001766//Apidaecin

UTERU3001988//TPR Domain

UTERU3002667//Polyomavirus coat protein

UTERU3002993//NOL1/NOP2/sun family

UTERU3003116//Urease//EGF-like domain

UTERU3003178//TPR Domain//TPR Domain//TPR Domain//TPR Domain//PPR repeat

UTERU3003523//PH domain//Fibroblast growth factor

UTERU3004616//Disintegrin

UTERU3004992//Immunoglobulin domain

UTERU3005460//Penicillin amidase//Bacterial regulatory proteins, lacI family

UTERU3005585//PDZ domain (Also known as DHR or GLGF).

UTERU3005907//Transglutaminase family

UTERU3006308//Integrins, beta chain//Plexin repeat//Immunoglobulin domain

UTERU3007419//PH domain

UTERU3007640//NSF attachment protein

UTERU3008660//TPR Domain//TPR Domain

UTERU3009490//Bromodomain

UTERU3009871//Ank repeat//Ank repeat//Ank repeat//Ank repeat//TPR Domain//Ank repeat//Ank repeat

UTERU3009979//EGF-like domain//EGF-like domain//EGF-like domain//Trypsin Inhibitor like cysteine rich domain//EGF-like domain//Laminin G domain//Thrombospondin N-terminal -like domains//Laminin G domain

UTERU3015500//Leucine rich repeat N-terminal domain//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat

Deduced amino acid sequences of following 250 clones were also detected to have functional domains with Pfam.

3NB692004724//KRAB box//Integrase core domain

ADRGL2000042//Nucleosome assembly protein (NAP)

BLADE2000579//Src homology domain 2//Peptidase family C9

BLADE2006830//HSF-type DNA-binding domain

BRACE2003609//Zinc finger, C2H2 type//Zinc finger, C2H2 type//Zinc finger, C2H2 type//Zinc finger, C2H2 type//PHD-finger//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type//Zinc finger, C2H2 type

BRACE2029396//Somatotropin hormone family

BRACE2037299//Integrase core domain

BRACE2039823//CDP-alcohol phosphatidyltransferase

BRACE3001058//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Putative zinc finger in N-recognin//Zinc finger, C2H2 type//Zinc finger, C2H2 type//Transcription factor S-II (TFIIS)//Src homology domain 2//Zinc finger, C2H2 type//Zinc finger, C2H2 type//Src homology domain 2//Zinc finger, C2H2 type

BRACE3001113//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Putative zinc finger in N-recognin//Zinc finger, C2H2 type//PHD-finger//Zinc finger, C2H2 type

BRACE3003026//Phorbol esters/diacylglycerol binding domain (C1 domain)//Zinc finger, C3HC4 type (RING finger)//PHD-finger

BRACE3003053//Influenza RNA-dependent RNA polymerase subunit//Reprolysin family propeptide//Leptin

BRACE3005107//Small cytokines (intecrine/chemokine), interleukin-8 like

BRACE3009127//PH domain//Oxysterol-binding protein

BRACE3010076//KH domain//KH domain//Domain of unknown function//KH domain//KH domain//KH domain//Small cytokines (intecrine/chemokine), interleukin-8 like//Fanconi anaemia group C protein//KH domain//KH domain

BRALZ2017844//Homeobox domain

BRAMY2019111//Ion transport protein

BRAMY2035070//Zinc finger, C2H2 type//Zinc finger, C2H2 type//PHD-finger//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

BRAMY2035449//KRAB box//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

BRAMY2035718//HMG (high mobility group) box//CTF/NF-I family

BRAMY2038516//Thioredoxin//Thioredoxin

BRAMY2039341//Zinc finger, C2H2 type//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type

BRAMY2040159//Chalcone and stilbene synthases//Adaptor complexes medium subunit family//PH domain//Putative GTP-ase activating protein for Arf//Ank repeat//Ank repeat

BRAMY2045471//DHHC zinc finger domain

BRAMY3004800//Synaptobrevin//RhoGEF domain

BRAWH1000369//DNA polymerase family A

BRAWH2006207//KRAB box

BRAWH2006395//Immunoglobulin domain//Thrombospondin type 1 domain

BRAWH2010552//Cyclin

BRAWH3007441//Zinc finger C-x8-C-x5-C-x3-H type (and similar)

BRAWH3009017//WD domain, G-beta repeat//WD domain, G-beta repeat

BRCAN2002473//Tropomyosins//Tropomyosins//UvrB/uvrC motif//Tropomyosins

BRCAN2002854//SAP domain

BRCAN2003070//Ubiquitin-conjugating enzyme

BRCAN2014229//SRP54-type protein//SRP54-type protein//Shikimate kinase//Adenylate kinase//ATPases associated with various cellular activities (AAA)

BRCOC2019841//Purple acid phosphatase

BRHIP2005724//alpha/beta hydrolase fold

BRHIP2006617//TPR Domain//TPR Domain

BRHIP2008389//Adenylate and Guanylate cyclase catalytic domain

BRHIP2012360//XPG N-terminal domain//XPG I-region

BRHIP2017553//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

BRHIP2026877//Eukaryotic protein kinase domain

BRHIP3000017//Integrins, beta chain//Uncharacterized protein family UPF0004

BRHIP3000240//Aldo/keto reductase family//Aldo/keto reductase family

BRHIP3008314//Sir2 family

BRHIP3026052//Protein phosphatase 2A regulatory B subunit (B56 family)

BRSTN2013354//Ets-domain

BRTHA2002133//Reverse transcriptase (RNA-dependent DNA polymerase)

BRTHA2002702//RNase H

BRTHA2007060//Transposase

BRTHA2010033//AP endonuclease family 1

BRTHA2013426//AP endonuclease family 1

BRTHA2013610//Deoxynucleoside kinase

BRTHA2016318//KE2 family protein

BRTHA2017364//DEAD/DEAH box helicase//Helicases conserved C-terminal domain

BRTHA2017972//Dwarfin

BRTHA2018011//Trypsin

BRTHA3000296//Peptidase family M20/M25/M40

CERVX2002013//Zinc finger, C2H2 type//Zinc finger, C2H2 type

CTONG1000113//KRAB box//Zinc finger, C2H2 type//Zinc finger, C2H2 type//Integrase core domain//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

CTONG2003348//bZIP transcription factor//Importin beta binding domain

CTONG2004000//Acyl-CoA dehydrogenase

CTONG2015596//Myb-like DNA-binding domain

CTONG2016942//Trypsin

CTONG2019822//Hepatitis C virus core protein

CTONG2020374//MORN motif//MORN motif//MORN motif//MORN motif//MORN motif//MORN motif//MORN motif//Penicillin amidase//Bacterial regulatory proteins, lacI family//Vacuolar sorting protein 9 (VPS9) domain

CTONG2020378//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

CTONG2020411//Zinc finger, C2H2 type//Zinc finger, C2H2 type//Zinc finger, C2H2 type//Zinc finger, C2H2 type//Zinc finger, C2H2 type//Zinc finger, C2H2 type

CTONG2024031//MORN motif//MORN motif//MORN motif//MORN motif//MORN motif//MORN motif//MORN motif//Penicillin amidase//Bacterial regulatory proteins, lacI family

CTONG2028758//Zinc finger, C2H2 type//Zinc finger, C2H2 type//Homeobox domain//Zinc finger, C2H2 type//Homeobox domain//Zinc finger, C2H2 type

CTONG3001501//Zinc finger, C2H2 type

CTONG3002552//AP endonuclease family 1

CTONG3003598//AP endonuclease family 1

CTONG3004550//Phosphoribosyl-ATP pyrophosphohydrolase//Death domain

CTONG3004726//KRAB box//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type//Zinc finger, C2H2 type

DFNES2011192//KRAB box//Zinc finger, C2H2 type//Zinc finger, C2H2 type//Zinc finger, C2H2 type//Zinc finger, C2H2 type//. Zinc finger, C2H2 type//Zinc finger, C2H2 type//Zinc finger, C2H2 type

FCBBF1000509//SAM domain (Sterile alpha motif)//SURF4 family//SAM domain (Sterile alpha motif)//DNA photolyase

FCBBF3010361//Fork head domain

FCBBF3027854//Zinc finger, C2H2 type

FEBRA2001990//RasGEF domain

FEBRA2006519//Thrombospondin type 1 domain//Thrombospondin type 1 domain

FEBRA2008692//PQQ enzyme repeat//wnt family of developmental signaling proteins//Eukaryotic protein kinase domain//Ribulose bisphosphate carboxylase, large chain

FEBRA2014122//Zinc finger, C2H2 type//Zinc finger, C2H2 type//Zinc finger, C2H2 type//Sodium//Zinc finger, C2H2 type

FEBRA2027609//Zinc finger, C2H2 type//Rubredoxin//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Putative zinc finger in N-recognin//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

FEBRA2028256//EGF-like domain//EGF-like domain//EGF-like domain//EGF-like domain//EGF-like domain//TB domain//EGF-like domain//EGF-like domain//EGF-like domain//EGF-like domain//EB module//Squash family of serine protease inhibitors//EGF-like domain//EGF-like domain

FEBRA2028516//GRIP domain

HCASM2002754//Sterol desaturase

HCASM2003018//PHD-finger//Zinc finger//Zinc finger//Zinc finger//Zinc finger//Zinc finger//Zinc finger//Zinc finger//Zinc finger//Zinc finger//Zinc finger//Zinc finger//Zinc finger

HCASM2003099//Histone deacetylase family//Zn-finger in ubiquitin-hydrolases and other proteins

HCASM2003357//Coproporphyrinogen III oxidase

HCASM2008536//XRCC1 N terminal domain

HCASM2009424//RFX DNA-binding domain

HCHON2000508//PH domain//bZIP transcription factor//bZIP transcription factor//Outer membrane efflux protein//Troponin

HCHON2000743//SCAN domain

HCHON2004858//SCAN domain//Myb-like DNA-binding domain//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Transcription factor S-II (TFIIS)//Zinc finger, C2H2 type

HEART2009680//Bacteriorhodopsin//7 transmembrane receptor (Secretin family)

HLUNG2015418//Cadherin domain//Cadherin domain//Cadherin domain//Cadherin domain//PQQ enzyme repeat

HLUNG2015548//IMP dehydrogenase/GMP reductase N terminus//CBS domain//CBS domain//Dihydroorotate dehydrogenase//Histidine biosynthesis protein//FMN-dependent dehydrogenase//Conserved region in glutamate synthase//IMP dehydrogenase/GMP reductase C terminus

HSYRA2005628//KRAB box//Zinc finger, C2H2 type//PHD-finger//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

IMR322007078//UBA domain

IMR322008651//Helix-hairpin-helix motif.

IMR322013396//Transmembrane region cyclic Nucleotide Gated Channel//Cyclic nucleotide-binding domain

IMR322013731//ATPases associated with various cellular activities (AAA)//Bromodomain

LIVER2000247//Sodium

MESAN2001770//Regulatory subunit of type II PKA R-subunit//eIF4-gamma/eIF5/eIF2-epsilon

MESAN2005303//Ank repeat//HECT-domain (ubiquitin-transferase).

MESAN2014412//Zinc finger, C2H2 type//Zinc finger, C2H2 type//Zinc finger, C2H2 type//Zinc finger, C2H2 type//Zinc finger, C2H2 type//Zinc finger, C2H2 type

MESAN2015501//Zinc finger, C2H2 type//Zinc finger, C2H2 type//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

NT2RI2005772//Guanylate kinase//I/LWEQ domain//PDZ domain (Also known as DHR or GLGF).//SH3 domain

NT2RI2008952//FYVE zinc finger//PHD-finger//Plant PEC family metallothionein//RNA polymerases M/15 Kd subunits//TRAF-type zinc finger//Transcription factor S-II (TFIIS)//Zinc finger, C2H2 type

NT2RI2009583//7 transmembrane receptor (metabotropic glutamate family)//GPR1/FUN34/yaaH family

NT2RI2018448//Zinc finger, C2H2 type//Zinc finger, C2H2 type//Zinc finger, C2H2 type

NT2RI2027157//Reeler domain//HSF-type DNA-binding domain

NT2RI3000174//SNF2 and others N-terminal domain//Small cytokines (intecrine/chemokine), interleukin-8 like//Helicases conserved C-terminal domain//Zn-finger in Ran binding protein and others.//HNH endonuclease

NT2RI3001132//Zinc finger, C3HC4 type (RING finger)//PHD-finger

NT2RI3002557//Ribosomal protein L36//bZIP transcription factor

NT2RI3007167//Cold-shock′ DNA-binding domain//Zinc knuckle//Zinc knuckle

NT2RI3007443//Alpha-2-macroglobulin family//Eukaryotic protein kinase domain//REV protein (anti-repression trans-activator protein)

NT2RP7008435//Anenome neurotoxin//CUB domain//Low-density lipoprotein receptor domain class A//Trypsin

NT2RP8000521//Small cytokines (intecrine/chemokine), interleukin-8 like

NTONG2008093//Adenylylsulfate kinase//6-phosphofructo-2-kinase

OCBBF2003327//Thrombospondin type 1 domain//Thrombospondin type 1 domain//Thrombospondin type 1 domain

OCBBF2005433//SH3 domain//WW domain//PH domain//RhoGAP domain

OCBBF2006987//Collagen triple helix repeat (20 copies)//Eukaryotic DNA topoisomerase I

OCBBF2008144//KRAB box//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type//PHD-finger//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

OCBBF2009583//KRAB box//Zinc finger, C2H2 type//Zinc finger, C2H2 type//Zinc finger, C2H2 type//Zinc finger, C2H2 type//Zinc finger, C2H2 type

OCBBF2011669//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Putative zinc finger in N-recognin//Zinc finger, C2H2 type//PHD-finger//Zinc finger, C2H2 type

OCBBF2019684//Zinc finger, C2H2 type//Zinc finger, C2H2 type//Transcription factor S-II (TFIIS)//Zinc finger, C2H2 type//Zinc finger, C2H2 type//PHD-finger//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

OCBBF2020048//Protein of unknown function DUF98//Zinc finger, C3HC4 type (RING finger)

OCBBF2024284//Hemagglutinin//PHD-finger//Zinc finger//Zinc finger//Zinc finger//Zinc finger//Zinc finger//Zinc finger//Zinc finger//Zinc finger//Zinc finger//Zinc finger//Zinc finger//Zinc finger//WD domain, G-beta repeat//WD domain, G-beta repeat//WD domain, G-beta repeat

OCBBF2030116//Hr1 repeat motif//Transthyretin precursor (formerly prealbumin)//Tau and MAP proteins, tubulin-binding//Transient receptor//Syntaxin

OCBBF2032274//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type

OCBBF2034637//Prokaryotic DNA topoisomerase//Protein of unknown function//Eukaryotic protein kinase domain

OCBBF3000167//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

OCBBF3002654//SH3 domain//Immunoglobulin domain//Eukaryotic protein kinase domain

OCBBF3003761//KH domain//KH domain//Zinc finger, C3HC4 type (RING finger)

PERIC2007068//ELM2 domain//Myb-like DNA-binding domain//Eukaryotic initiation factor 1A//Myb-like DNA-binding domain

PLACE7000333//AP endonuclease family 1

PLACE7000502//Ank repeat//Ank repeat//Ank repeat//Ank repeat//Ank repeat//Peptidase family C9

PROST2000452//Trypsin

PROST2009320//LIM domain containing proteins//LIM domain containing proteins

PUAEN2006335//Formin Homology 2 Domain

SKMUS2003194//SAP domain

SPLEN2016135//Zinc finger, C2H2 type//Zinc finger, C2H2 type//Phorbol esters/diacylglycerol binding domain (C1 domain)//Zinc finger, C2H2 type

SPLEN2016781//Transcription factor S-II (TFIIS)//Zinc finger, C2H2 type//Zinc finger, C2H2 type//Zinc finger, C2H2 type

SPLEN2030847//Kinesin motor domain//Kinesin motor domain//GGL domain

SPLEN2036702//REJ domain//Phorbol esters/diacylglycerol binding domain (C1 domain)//PHD-finger

SPLEN2039311//dUTPase

SPLEN2039379//Transthyretin precursor (formerly prealbumin)

STOMA2003158//Deoxyribonuclease I (DNase I)

SYNOV1000256//Leucine Rich Repeat//BAH domain//Leucine Rich Repeat//Leucine Rich Repeat//Leucine Rich Repeat

SYNOV2006620//Nuclear transition protein 2

SYNOV2013637//Chalcone and stilbene synthases

SYNOV2021953//Zinc finger, C2H2 type//Zinc finger, C2H2 type//Zinc finger, C2H2 type//Zinc finger, C2H2 type

SYNOV4002744//Zinc finger, C2H2 type//Zinc finger, C2H2 type//Zinc finger, C2H2 type//Zinc finger, C2H2 type

SYNOV4003981//Somatomedin B domain//WAP-type (Whey Acidic Protein) ‘four-disulfide core’//Hemopexin//Hemopexin

SYNOV4005889//Apolipoprotein A1/A4/E family

TESOP2000390//Eukaryotic protein kinase domain

TESOP2001796//Zinc finger, C3HC4 type (RING finger)//PHD-finger//IBR domain

TESOP2005199//KRAB box//Zinc finger, C2H2 type//Zinc finger, C2H2 type//Zinc finger, C2H2 type//Transcription factor S-II (TFIIS)//Zinc finger, C2H2 type//Zinc finger, C2H2 type//PHD-finger//Zinc finger, C2H2 type//Zinc finger, C2H2 type//Zinc finger, C2H2 type//Zinc finger, C2H2 type//TRAF-type zinc finger//Zinc finger, C2H2 type

TESOP2006398//Zinc finger, C2H2 type

TESOP2006865//KRAB box

TESTI1000266//Integrase core domain

TESTI2008901//Transcription factor WhiB

TESTI2015626//Phosphoribosyl transferase domain

TESTI2025924//Eukaryotic protein kinase domain

TESTI2026647//DEAD/DEAH box helicase//Helicases conserved C-terminal domain

TESTI2029252//Ion transport protein//Polysaccharide biosynthesis protein

TESTI2034251//Zinc finger, C2H2 type//Zinc finger, C2H2 type//Zinc finger, C2H2 type//Zinc finger, C2H2 type//Transcription factor S-II (TFIIS)//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

TESTI2035981//RNA polymerase alpha subunit

TESTI2036288//Aldo/keto reductase family

TESTI2037830//Zinc finger, C2H2 type//Zinc finger, C2H2 type

TESTI2039060//D-ala D-ala ligase//Glycosyl hydrolases family 31

TESTI2049956//WD domain, G-beta repeat//WD domain, G-beta repeat//PQQ enzyme repeat//WD domain, G-beta repeat//WD domain, G-beta repeat//WD domain, G-beta repeat//WD domain, G-beta repeat

TESTI2050780//Kazal-type serine protease inhibitor domain

TESTI4000137//Domain of unknown function

TESTI4000155//Viral RNA dependent RNA polymerase

TESTI4000183//KRAB box//Zinc finger, C2H2 type//Zinc finger, C2H2 type//Zinc finger, C2H2 type

TESTI4000214//Zinc finger, C3HC4 type (RING finger)//DENN (AEX-3) domain

TESTI4000319//RasGEF domain

TESTI4001984//Retroviral aspartyl protease//G-patch domain

TESTI4005317//Bacterial flagellin C-terminus//Phosphotyrosine interaction domain (PTB/PID)

TESTI4006473//Sigma-54 transcription factors//DEAD/DEAH box helicase//DEAD/DEAH box helicase//Ank repeat//Ank repeat//Helicases conserved C-terminal domain

TESTI4008058//Zn-finger in Ran binding protein and others.//Zinc finger, CCHC class

TESTI4008302//Zinc finger, C2H2 type//Zinc finger, C2H2 type//Zinc finger, C2H2 type//Zinc finger, C2H2 type//PHD-finger//Zinc finger, C2H2 type//Zinc finger, C2H2 type//Zinc finger, C2H2 type//Zinc finger, C2H2 type//Zinc finger, C2H2 type

TESTI4010382//Luteovirus (ORF3) RNA-directed RNA-polymerase//Ezrin/radixin/moesin family

TESTI4011072//Tudor domain//Tudor domain//Staphylococcal nuclease homologues//Tudor domain//Tudor domain

TESTI4013365//MYND finger

TESTI4013894//Synaptophysin/synaptoporin

TESTI4014801//Zinc finger, C2H2 type//N2,N2-dimethylguanosine tRNA methyltransferase

TESTI4015442//Homeobox domain//Zinc finger, C2H2 type//Homeobox domain//Zinc finger, C2H2 type//Zinc finger, C2H2 type//Zinc finger, C2H2 type

TESTI4017714//Transcriptional regulatory protein, C terminal//Prolyl oligopeptidase family

TESTI4021482//Eukaryotic protein kinase domain

TESTI4024387//GDP dissociation inhibitor

TESTI4025268//WD domain, G-beta repeat//WD domain, G-beta repeat

TESTI4025494//KRAB box//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//TRAF-type zinc finger//Zinc finger, C2H2 type//Zinc finger, C2H2 type//Zinc finger, C2H2 type//Zinc finger, C2H2 type//Zinc finger, C2H2 type

TESTI4025547//Type II intron maturase//Zinc finger, C2H2 type//Zinc finger, C2H2 type//Zinc finger, C2H2 type

TESTI4025865//Lipoate-protein ligase B//KE2 family protein

TESTI4026207//Kinesin motor domain//DNA gyrase/topoisomerase IV, subunit A

TESTI4028938//KRAB box//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//DM DNA binding domain//Zinc finger, C2H2 type//Zinc finger, C2H2 type

TESTI4028958//DNA gyrase/topoisomerase IV, subunit A//Apolipoprotein A1/A4/E family

TESTI4029348//Trans-activation protein X

TESTI4029528//RanBP1 domain.

TESTI4029690//Leucine Rich Repeat//Leucine Rich Repeat//Leucine Rich Repeat//Cytochrome c oxidase subunit VIa//IQ calmodulin-binding motif

TESTI4031745//Alpha-2-macroglobulin family//Eukaryotic protein kinase domain//REV protein (anti-repression trans-activator protein)

TESTI4032090//RNase H//Integrase Zinc binding domain//Integrase core domain

TESTI4032112//Syndecan domain

TESTI4038721//Squash family of serine protease inhibitors

TESTI4041086//Transmembrane amino acid transporter protein

TESTI4046240//Sir2 family

THYMU2004139//Eukaryotic protein kinase domain

THYMU2004284//Repeat in ubiquitin-activating (UBA) proteins

THYMU2006001//Zinc finger, C3HC4 type (RING finger)//CONSTANS family zinc finger//B-box zinc finger.//SPRY domain

THYMU2028739//SCAN domain//KRAB box//Myb-like DNA-binding domain

THYMU2031139//Reprolysin (M12B) family zinc metalloprotease//Thrombospondin type 1 domain//EB module//Plant PEC family metallothionein//A20-like zinc finger

THYMU2031249//C-type lysozyme/alpha-lactalbumin family//Eukaryotic protein kinase domain

THYMU2035078//Domain of unknown function DUF27

THYMU2035710//ATP1G1/PLM/MAT8 family

THYMU2040925//CDP-alcohol phosphatidyltransferase

THYMU3000269//FAD binding domain

THYMU3000360//Integrase core domain

THYMU3001428//Zinc finger, C3HC4 type (RING finger)//PHD-finger

TKIDN2012771//DNA polymerase (viral) C-terminal domain//MttB family UPF0032

TLIVE2001684//Alpha-2-macroglobulin family//Alpha-2-macroglobulin family

TLIVE2002046//HMG (high mobility group) box//Uroporphyrinogen decarboxylase (URO-D)//Delta-aminolevulinic acid dehydratase

TLIVE2007607//DNA polymerase (viral) C-terminal domain//Cytochrome P450

TRACH1000212//TSC-22/dip/bun family

TRACH2000862//Guanylate-binding protein

TRACH2007483//Zinc finger, C2H2 type//Zinc finger, C2H2 type//Zinc finger, C2H2 type//Zinc finger, C2H2 type//Zinc finger, C2H2 type

TRACH2019672//CRAL/TRIO domain.

TRACH2024408//Death domain

TRACH2024559//IQ calmodulin-binding motif//IQ calmodulin-binding motif

TRACH3000134//KRAB box//Zinc finger, C2H2 type//Zinc finger, C2H2 type//Zinc-finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type//PHD-finger//Zinc finger, C2H2 type//Zinc finger, C2H2 type//Zinc finger, C2H2 type//DnaJ central domain (4 repeats)//Zinc finger, C2H2 type//Zinc finger, C2H2 type//Zinc finger, C2H2 type//Zinc finger, C2H2 type//Zinc finger, C2H2 type//Zinc finger, C2H2 type//Zinc finger, C2H2 type

TRACH3000420//ABC transporter//Papain family cysteine protease//ABC transporter

TRACH3002561//‘Cold-shock’ DNA-binding domain

TRACH3003832//PHD-finger

TRACH3007866//Transcriptional regulatory protein, C terminal//PAC motif//Dipeptidyl peptidase IV (DPP IV) N-terminal region//Prolyl oligopeptidase family

UTERU2004299//ATP P2X receptor

UTERU2008040//Phorbol esters/diacylglycerol binding domain (C1 domain)//SH3 domain

UTERU2019534//Cysteine rich repeat//Cysteine rich repeat//Carboxylesterases

UTERU2028734//C2 domain//C2 domain

UTERU2032279//Serpins (serine protease inhibitors)

UTERU2033577//KRAB box

UTERU3000402//WD domain, G-beta repeat//WD domain, G-beta repeat

UTERU3000738//Eukaryotic protein kinase domain

UTERU3001053//Zinc finger, C2H2 type//Zinc finger, C2H2 type//Zinc finger, C2H2 type//Zinc finger, C2H2 type//Zinc finger, C2H2 type//Zinc finger, C2H2 type//Transcription factor S-II (TFIIS)//Zinc finger, C2H2 type//Zinc finger, C2H2 type

UTERU3014791//LIM domain containing proteins//PHD-finger//LIM domain containing proteins

UTERU3015412//WD domain, G-beta repeat//WD domain, G-beta repeat//WD domain, G-beta repeat//Keratin, high sulfur B2 protein

UTERU3017176//K-box region//bZIP transcription factor

TESTI4038779//K+channel tetramerisation domain//BTB/POZ domain//Zinc finger, C2H2 type//Zinc finger, C2H2 type//Rubredoxin//PHD-finger//Zinc finger, C2H2 type

### EXAMPLE 6

### Functional Categorization Based on Homology Search of the Full-Length Nucleotide Sequences and Deduced Amino Acid Sequences

The functional prediction and categorization of the proteins encoded by the clones were carried out based on the result of homology search of the databases of GenBank, Swiss-Prot, UniGene, nr, and RefSeq (see the Homology Search Result Data) for the full-length nucleotide sequences and deduced amino acid sequences.

A clone predicted to belong to the category of secretory protein/membrane protein means a clone having hit data with some annotation, such as growth factor, cytokine, hormone, signal, transmembrane, membrane, extracellular matrix, receptor, G-protein coupled receptor, ionic channel, voltage-gated channel, calcium channel, cell adhesion, collagen, and connective tissue, suggesting that it is a secretory or membrane protein, or means a clone in which the presence of nucleotide sequence encoding a signal sequence or transmembrane domain was suggested by the results of PSORT and SOSUI analyses for deduced ORF.

A clone predicted to belong to the category of glycoprotein-related protein means a clone having hit data with some annotation, such as glycoprotein, suggesting that the clone encodes a glycoprotein-related protein.

A clone predicted to belong to the category of signal transduction-related protein means a clone having hit data with some annotation, such as serine/threonine-protein kinase, tyrosine-protein kinase, SH3 domain, and SH2 domain, suggesting that the clone encodes a signal transduction-related protein.

A clone predicted to belong to the category of transcription-related protein means a clone having hit data with some annotation, such as transcription regulation, zinc finger, and homeobox, suggesting that the clone encodes a transcription-related protein.

A clone predicted to belong to the category of disease-related protein means a clone having hit data with some annotation, such as disease mutation and syndrome, suggesting that the clone encodes a disease-related protein, or means a clone whose full-length nucleotide sequence has hit data for Swiss-Prot, GenBank, or UniGene, where the hit data corresponds to genes or proteins which have been deposited in the Online Mendelian Inheritance in Man (OMIM) (http://www.ncbi.nlm.nih.gov/Omim/), which is the human gene and disease database.

A clone predicted to belong to the category of enzyme and/or metabolism-related protein means a clone having hit data with some annotation, such as metabolism, oxidoreductase, and E. C. No. (Enzyme commission number), suggesting that the clone encodes an enzyme and/or metabolism-related protein.

A clone predicted to belong to the category of cell division and/or cell proliferation-related protein means a clone having hit data with some annotation, such as cell division, cell cycle, mitosis, chromosomal protein, cell growth, and apoptosis, suggesting that the clone encodes a cell division and/or cell proliferation-related protein.

A clone predicted to belong to the category of cytoskeleton-related protein means a clone having hit data with some annotation, such as structural protein, cytoskeleton, actin-binding, and microtubles, suggesting that the clone encodes a cytoskeleton-related protein.

A clone which is predicted to belong to the category of nuclear protein and/or RNA synthesis-related protein means a clone having hit data with some annotation, such as nuclear protein, RNA splicing, RNA processing, RNA helicase, and polyadenylation, suggesting that the clone encodes a nuclear protein and/or RNA synthesis-related protein.

A clone predicted to belong to the category of protein synthesis and/or transport-related protein means a clone having hit data with some annotation, such as translation regulation, protein biosynthesis, amino-acid biosynthesis, ribosomal protein, protein transport, and signal recognition particle, suggesting that the clone encodes a protein synthesis and/or transport-related protein.

A clone predicted to belong to the category of cellular defense-related protein means a clone having hit data with some annotation, such as heat shock, DNA repair, and DNA damage, suggesting that the clone encodes a cellular defense-related protein.

A clone predicted to belong to the category of development and/or differentiation-related proteins means a clone having hit data with some annotation, such as developmental protein, suggesting that the clone encodes a development and/or differentiation-related protein.

A clone predicted to belong to the category of DNA-binding and/or RNA-binding protein means a clone having hit data with some annotation, such as DNA-binding, RNA-binding, etc.

A clone predicted to belong to the category of ATP-binding and/or GTP-binding protein means a clone having hit data with some annotation, such as ATP-binding, GTP-binding, etc.

In this functional categorization, when a single clone corresponded to multiple categories of those shown above, the clone was assigned to the multiple categories. However, the function of a protein is not restricted to the functional categories in this classification, and there is the possibility that other functions are newly assigned to the protein.

The clones predicted to belong to the category of secretory protein and/or membrane protein are the following 659 clones.

ACTVT2000380, ADIPS2000088, ADRGL2000172, ADRGL2003329, ADRGL2009146, ASTRO2014923, ASTRO3000301, BLADE1000176, BLADE2002073, BLADE2002947, BLADE2004462, BLADE2004670, BLADE2005036, BLADE2008539, BNGH42003570, BRACE1000186, BRACE2005457, BRACE2014306, BRACE2016981, BRACE2029112, BRACE2030884, BRACE2031527, BRACE2031531, BRACE2031899, BRACE2032385, BRACE2036005, BRACE2039249, BRACE2039327, BRACE2040138, BRACE2041200, BRACE2043142, BRACE2043665, BRACE2046295, BRACE3000697, BRACE3001391, BRACE3002298, BRACE3003004, BRACE3003595, BRACE3004058, BRACE3004113, BRACE3004772, BRACE3004843, BRACE3006462, BRACE3008137, BRACE3008384, BRACE3009574, BRACE3009708, BRACE3010397, BRACE3011271, BRACE3011505, BRACE3013740, BRACE3014005, BRACE3014068, BRACE3014807, BRACE3016884, BRACE3018963, BRACE3019084, BRACE3020286, BRACE3020594, BRACE3024662, BRACE3025531, BRACE3025630, BRACE3026008, BRACE3026735, BRACE3027326, BRACE3031838, BRACE3040856, BRALZ2016085, BRAMY2001473, BRAMY2004771, BRAMY2005052, BRAMY2017528, BRAMY2019300, BRAMY2019963, BRAMY2021498, BRAMY2028856, BRAMY2033003, BRAMY2033116, BRAMY2033594, BRAMY2036396, BRAMY2039872, BRAMY2040592, BRAMY2041542, BRAMY2045036, BRAMY2047420, BRAMY2047751, BRAMY2047765, BRAMY3002312, BRAMY3004224, BRAMY3004919, BRAMY3007206, BRAMY3007609, BRAMY3008505, BRAMY4000095, BRASW1000125, BRAWH1000127, BRAWH2002560, BRAWH2002761, BRAWH2007658, BRAWH2014414, BRAWH2014954, BRAWH2016221, BRAWH2016439, BRAWH2016702, BRAWH3000078, BRAWH3000314, BRAWH3001475, BRAWH3001891, BRAWH3002600, BRAWH3003555, BRAWH3003727, BRAWH3003992, BRAWH3004453, BRAWH3004666, BRAWH3005132, BRAWH3005912, BRAWH3006548, BRAWH3007221, BRAWH3007506, BRAWH3007592, BRAWH3008634, BRCAN2002948, BRCAN2006063, BRCAN2009203, BRCAN2010376, BRCAN2012355, BRCAN2012481, BRCAN2013655, BRCAN2014143, BRCAN2016619, BRCAN2024451, BRCOC2007034, BRCOC2019934, BRHIP2000691, BRHIP2001805, BRHIP2002172, BRHIP2004814, BRHIP2004883, BRHIP2005236, BRHIP2005752, BRHIP2009414, BRHIP2013699, BRHIP2026288, BRHIP3000526, BRHIP3007483, BRHIP3007586, BRHIP3008598, BRHIP3009448, BRHIP3015751, BRHIP3024118, BRHIP3026097, BRSSN2003086, BRSSN2004496, BRSSN2008549, BRSSN2011738, BRSSN2014424, BRSSN2018925, BRSTN2000872, BRSTN2003835, BRSTN2007000, BRSTN2010363, BRSTN2012380, BRSTN2015015, BRSTN2016470, BRSTN2016678, BRSTN2017110, BRTHA2002376, BRTHA2002493, BRTHA2002608, BRTHA2002808, BRTHA2003110, BRTHA2003461, BRTHA2005579, BRTHA2006075, BRTHA2008527, BRTHA2011194, BRTHA2012980, BRTHA2013460, BRTHA2015696, BRTHA2015878, BRTHA2016215, BRTHA2016496, BRTHA2017985, BRTHA2018344, BRTHA2018624, BRTHA3000633, BRTHA3002427, BRTHA3003474, BRTHA3007148, BRTHA3008386, BRTHA3008778, BRTHA3009037, BRTHA3009090, BRTHA3009291, BRTHA3016845, BRTHA3017047, BRTHA3017589, BRTHA3017848, BRTHA3018656, CERVX2002006, COLON2000568, COLON2002443, COLON2004478, COLON2005126, COLON2005772, CTONG1000302, CTONG1000341, CTONG1000488, CTONG1000508, CTONG2000042, CTONG2004062, CTONG2008233, CTONG2009423, CTONG2009531, CTONG2010803, CTONG2013178, CTONG2019652, CTONG2019788, CTONG2020127, CTONG2020522, CTONG2020638, CTONG2022601, CTONG2023512, CTONG2024749, CTONG2025496, CTONG2026920, CTONG2027327, CTONG2028124, CTONG2028687, CTONG3000707, CTONG3001370, CTONG3001560, CTONG3002020, CTONG3003179, CTONG3003483, CTONG3003737, CTONG3005648, CTONG3008252, CTONG3008258, CTONG3008496, CTONG3008566, CTONG3008951, CTONG3009227, CTONG3009239, CTONG3009328, CTONG3009385, D3OST2002182, D3OST2002648, DFNES1000107, DFNES2000146, DFNES2005266, DFNES2010502, FCBBF2001183, FCBBF2007510, FCBBF3003435, FCBBF3004502, FCBBF3009888, FCBBF3012170, FCBBF3021576, FCBBF3023895, FCBBF4000076, FEBRA1000030, FEBRA2007708, FEBRA2008311, FEBRA2008468, FEBRA2020668, FEBRA2025427, FEBRA2027082, HCASM2002502, HCASM2003212, HCASM2007047, HCHON2000212, HCHON2001084, HCHON2001548, HCHON2001577, HCHON2001712, HCHON2002676, HCHON2004007, HCHON2004776, HCHON2005921, HEART1000010, HEART2001680, HEART2010492, HLUNG2000014, HLUNG2003872, HLUNG2010464, HLUNG2015617, HLUNG2017350, HSYRA2005496, HSYRA2006873, HSYRA2008714, HSYRA2009102, IMR322002110, IMR322006222, KIDNE1000064, KIDNE2000832, KIDNE2000846, KIDNE2006580, KIDNE2010264, KIDNE2011635, KIDNE2012945, KIDNE2013095, LIVER2007415, LYMPB2000083, MESAN2001979, MESAN2012054, MESTC1000042, NHNPC2000606, NHNPC2001223, NOVAR2000136, NOVAR2001108, NT2RI2008724, NT2RI2009855, NT2RI2025909, NT2RI3001263, NT2RI3003095, NT2RI3003382, NT2RI3003409, NT2RI3005403, NT2RI3006171, NT2RI3006673, NT2RI3007065, NT2RI3007543, NT2RI3007978, NT2RP7000359, NT2RP7000466, NT2RP7004027, NT2RP7009030, NT2RP7014005, NTONG2000413, OCBBF2006151, OCBBF2006567, OCBBF2006764, OCBBF2007114, OCBBF2007428, OCBBF2009926, OCBBF2010140, OCBBF2017516, OCBBF2021788, OCBBF2024719, OCBBF2025458, OCBBF2030517, OCBBF2030574, OCBBF2031167, OCBBF2032590, OCBBF2033869, OCBBF2037598, OCBBF2038317, OCBBF3000483, OCBBF3003320, OCBBF3004314, PEBLM2000170, PEBLM2000338, PEBLM2002594, PEBLM2006113, PEBLM2007834, PERIC2001227, PERIC2003452, PERIC2003720, PERIC2004909, PERIC2005347, PERIC2006035, PERIC2007914, PLACE5000171, PLACE5000260, PLACE5000282, PLACE6012574, PLACE6019932, PLACE6020031, PLACE7000514, PLACE7001022, PROST1000184, PROST1000528, PROST1000559, PROST2003428, PROST2018090, PROST2018902, PROST2018922, PUAEN2002489, PUAEN2005588, PUAEN2006701, PUAEN2009174, PUAEN2009795, RECTM2000433, RECTM2001347, SKMUS2000757, SKNMC2002402, SMINT2002743, SMINT2009902, SMINT2011888, SMINT2015787, SPLEN2001599, SPLEN2009548, SPLEN2012889, SPLEN2015158, SPLEN2015267, SPLEN2015679, SPLEN2021701, SPLEN2023733, SPLEN2023791, SPLEN2025491, SPLEN2029522, SPLEN2029683, SPLEN2030335, SPLEN2030479, SPLEN2031125, SPLEN2031424, SPLEN2031547, SPLEN2031724, SPLEN2031780, SPLEN2032813, SPLEN2033098, SPLEN2034021, SPLEN2034781, SPLEN2036326, SPLEN2036821, SPLEN2037722, SPLEN2038180, SPLEN2038345, SPLEN2038407, SPLEN2040222, SPLEN2041304, SPLEN2042598, STOMA2004294, STOMA2008546, SYNOV2005817, SYNOV2012326, SYNOV2014400, SYNOV2016124, SYNOV4002883, SYNOV4003322, SYNOV4004184, SYNOV4004741, SYNOV4004914, SYNOV4006256, SYNOV4007430, SYNOV4007553, SYNOV4007671, SYNOV4008336, SYNOV4008440, TBAES2001258, TCERX2000613, TESOP2001345, TESOP2001865, TESOP2002273, TESOP2002539, TESOP2004114, TESOP2005485, TESOP2005579, TESOP2006041, TESOP2007052, TESOP2007262, TESOP2007636, TESTI1000257, TESTI1000348, TESTI2000644, TESTI2002036, TESTI2002618, TESTI2002928, TESTI2003347, TESTI2005610, TESTI2006648, TESTI2013382, TESTI2024567, TESTI2027019, TESTI2034767, TESTI2034953, TESTI2034997, TESTI2035997, TESTI2036684, TESTI2042450, TESTI2047071, TESTI2048898, TESTI2051767, TESTI2052822, TESTI4000215, TESTI4000724, TESTI4001100, TESTI4001527, TESTI4001561, TESTI4001665, TESTI4001923, TESTI4002552, TESTI4002754, TESTI4005805, TESTI4005961, TESTI4006053, TESTI4006137, TESTI4007064, TESTI4007163, TESTI4007239, TESTI4007382, TESTI4008050, TESTI4008401, TESTI4008429, TESTI4008797, TESTI4009608, TESTI4012448, TESTI4013369, TESTI4013667, TESTI4013830, TESTI4014392, TESTI4016238, TESTI4017575, TESTI4017901, TESTI4018835, TESTI4019566, TESTI4020092, TESTI4020102, TESTI4021478, TESTI4023722, TESTI4024420, TESTI4024874, TESTI4024890, TESTI4025797, TESTI4026456, TESTI4026785, TESTI4027821, TESTI4028062, TESTI4028429, TESTI4028823, TESTI4028880, TESTI4029836, TESTI4030159, TESTI4030505, TESTI4034172, TESTI4035065, TESTI4035649, TESTI4037244, TESTI4041053, TESTI4042711, TESTI4046487, TESTI4046819, THYMU2001053, THYMU2003632, THYMU2003760, THYMU2005003, THYMU2005303, THYMU2005321, THYMU2007658, THYMU2008725, THYMU2009425, THYMU2011548, THYMU2013386, THYMU2014353, THYMU2019210, THYMU2023711, THYMU2027497, THYMU2027695, THYMU2029676, THYMU2030068, THYMU2032035, THYMU2032437, THYMU2032655, THYMU2033079, THYMU2033308, THYMU2033816, THYMU2034314, THYMU2035064, THYMU2036085, THYMU2036459, THYMU2037226, THYMU2037348, THYMU2038772, THYMU2038797, THYMU2039780, THYMU2040412, THYMU2041015, THYMU3000028, THYMU3000036, THYMU3004835, THYMU3005696, THYMU3006168, THYMU3006811, THYMU3007137, THYMU3007368, THYMU3007845, TKIDN2002424, TKIDN2002632, TKIDN2006525, TKIDN2009092, TKIDN2009889, TKIDN2014771, TKIDN2019116, TLIVE2000023, TLIVE2001828, TLIVE2001927, TLIVE2002336, TLIVE2002690, TLIVE2003381, TLIVE2004110, TLIVE2008229, TOVAR2001281, TRACH1000205, TRACH2001549, TRACH2001684, TRACH2006387, TRACH2007059, TRACH2007834, TRACH2008300, TRACH2020525, TRACH2021964, TRACH2022553, TRACH2025535, TRACH2025911, TRACH3000014, TRACH3002064, TRACH3002168, TRACH3002650, TRACH3004786, TRACH3005294, TRACH3005549, TRACH3006149, TRACH3007391, TRACH3008629, TRACH3035199, TRACH3035526, TRACH3036193, TSTOM2000442, TSTOM2000553, TUTER2000916, UTERU1000339, UTERU2004688, UTERU2004929, UTERU2006137, UTERU2006568, UTERU2007444, UTERU2017762, UTERU2020718, UTERU2022020, UTERU2025025, UTERU2025645, UTERU2025891, UTERU2026090, UTERU2026203, UTERU2027591, UTERU2029953, UTERU2031851, UTERU2035323, UTERU2035469, UTERU3000645, UTERU3000899, UTERU3001240, UTERU3001571, UTERU3001585, UTERU3001652, UTERU3001988, UTERU3002209, UTERU3002383, UTERU3002786, UTERU3003116, UTERU3003776, UTERU3006308, UTERU3008671, UTERU3009690, UTERU3009979, UTERU3011063, UTERU3015500, UTERU3016789

The following 82 clones are also predicted to belong to the category of secretory protein and/or membrane protein.

BLADE2006830, BRACE2002589, BRACE2009318, BRACE2011677, BRACE2029396, BRACE2039823, BRACE2039832, BRAMY2019111, BRAMY2038516, BRAMY2045471, BRAWH2006395, BRAWH2008993, BRCOC2019841, BRHIP2003272, BRHIP2005271, BRHIP2005724, BRHIP2008389, BRHIP2026877, BRHIP3000240, BRTHA2011321, BRTHA2018011, BRTHA2018443, BRTHA3008826, CTONG2015633, CTONG2016942, CTONG2019822, FEBRA2000790, FEBRA2006519, FEBRA2028256, FEBRA2028516, HCASM2002754, HEART2009680, HLUNG2013350, HLUNG2015418, IMR322013396, LIVER2000247, NT2RI2009583, NT2RI2027157, NT2RP7008435, OCBBF2003327, OCBBF2030116, PLACE7000502, PROST2000452, PROST2019487, SPLEN2016932, SPLEN2037319, SYNOV2001660, SYNOV2013637, SYNOV4003981, SYNOV4005889, TBAES2000932, TESTI2015626, TESTI2029252, TESTI2032643, TESTI2039060, TESTI2050780, TESTI4000137, TESTI4000155, TESTI4006473, TESTI4011070, TESTI4013365, TESTI4013894, TESTI4014801, TESTI4032090, TESTI4041086, THYMU2004284, THYMU2030462, THYMU2033401, THYMU2034279, THYMU2035710, THYMU2040925, TKIDN2008778, TKIDN2012771, TKIDN2018926, TLIVE2007607, TRACH2019672, TRACH3000420, TRACH3003683, UTERU2011220, UTERU2021820, UTERU2032279, UTERU3015069

The clones predicted to belong to the category of glycoprotein-related protein are the following 115 clones.

ADIPS2000088, BNGH42003570, BRACE2005457, BRACE2014306, BRACE2029112, BRACE2039249, BRACE2046295, BRACE3001391, BRACE3011271, BRACE3016884, BRAMY2005052, BRAMY3004919, BRAMY4000095, BRAMY4000277, BRAWH1000127, BRAWH2007658, BRAWH2014414, BRAWH2016221, BRAWH3002600, BRCAN2006063, BRSSN2004496, BRTHA2008527, BRTHA2012980, BRTHA2016496, BRTHA3002427, BRTHA3017848, COLON2000568, COLON2004478, COLON2005772, CTONG1000341, CTONG2000042, CTONG2009423, CTONG2023512, CTONG2024749, CTONG2025496, CTONG3001370, CTONG3003737, D3OST2002648, DFNES2000146, DFNES2005266, FCBBF3012170, FEBRA1000030, FEBRA2008311, FEBRA2008468, HCHON2001712, HEART1000010, HEART2001680, HSYRA2005496, KIDNE2012945, LYMPB2000083, NESOP2001433, NOVAR2000136, NOVAR2001108, NT2RI3006171, NT2RI3006673, NT2RP7004027, OCBBF2033869, PLACE5000171, PROST1000184, PUAEN2009795, SMINT2010076, SMINT2011888, SMINT2015787, SPLEN2015267, SPLEN2021701, SPLEN2030335, SYNOV2005817, SYNOV2014400, SYNOV3000231, SYNOV3000302, TESOP2004114, TESOP2005485, TESTI1000257, TESTI2002036, TESTI2002618, TESTI2024567, TESTI2027019, TESTI4001527, TESTI4007163, TESTI4012406, TESTI4013830, TESTI4020092, TESTI4023546, TESTI4028823, TESTI4028880, TESTI4046819, THYMU2005303, THYMU2008725, THYMU2009425, THYMU2011548, THYMU2019210, THYMU2023711, THYMU2027497, THYMU2027695, THYMU2038797, THYMU3004835, TLIVE2003381, TRACH2006387, TRACH2007059, TRACH2022425, TRACH2022553, TRACH2022649, TRACH3002168, TRACH3008629, TRACH3035526, TSTOM2000442, UTERU2008347, UTERU2025025, UTERU2035469, UTERU3000899, UTERU3001240, UTERU3003116, UTERU3006308, UTERU3008671, UTERU3015500

The following 15 clones are also predicted to belong to the category of glycoprotein-related protein.

BRAMY2019111, BRHIP2026877, BRTHA2018011, FEBRA2028256, HEART2009680, HLUNG2015418, NT2RI2009583, NT2RP7008435, OCBBF2003327, TESTI2032643, TESTI2039060, TESTI4011070, THYMU2035710, TRACH3003683, UTERU2032279

The clones predicted to belong to the category of signal transduction-related protein are the following 80 clones.

BNGH42007788, BRACE2008594, BRACE2030341, BRACE2044286, BRACE3002508, BRACE3003595, BRACE3006872, BRACE3011421, BRACE3015027, BRACE3027326, BRAMY2036567, BRAMY2038904, BRAMY3000213, BRAMY3002803, BRAMY3005091, BRAMY3005932, BRAMY4000095, BRAMY4000229, BRCAN2003703, BRCAN2014602, BRCAN2016619, BRCAN2028355, BRHIP2000819, BRHIP3025161, BRSSN2004719, BRSTN2008418, BRTHA2002281, BRTHA2015406, CTONG2006798, CTONG3000084, CTONG3002412, D3OST3000169, FCBBF3007540, HCASM2001301, HCHON2006250, HCHON2008112, HLUNG2002465, KIDNE2001847, NESOP2001694, NT2NE2003252, NT2RI2005166, NT2RI3007757, NT2RI3008652, NT2RP7005529, NT2RP7009147, NT2RP7013795, NT2RP8000483, OCBBF2004826, OCBBF2007028, OCBBF2022351, OCBBF2030354, OCBBF2037547, PLACE6019385, PLACE7008431, PROST2016462, PROST2018511, PUAEN2009852, SPLEN2036932, SYNOV2021320, TESOP2000801, TESOP2001166, TESTI2005739, TESTI2026505, TESTI2050137, TESTI4011745, TESTI4012505, TESTI4018208, TESTI4028059, THYMU2007060, THYMU2031046, THYMU2032014, THYMU2039305, THYMU3008436, TLIVE2001327, TRACH2009310, TRACH2025535, TRACH3009455, UTERU2025025, UTERU2036089, UTERU3016789

The following 31 clones are also predicted to belong to the category of signal transduction-related protein.

BRAMY3004800, BRAWH3009017, BRHIP2026877, BRTHA2013610, BRTHA2017972, BRTHA3003000, CTONG2020974, FEBRA2001990, FEBRA2008692, NT2RI2005772, NT2RI3007443, NTONG2008093, OCBBF2005433, OCBBF2024284, OCBBF2034637, OCBBF3002654, SPLEN2036702, SPLEN2039379, TESOP2000390, TESTI2025924, TESTI2049956, TESTI4000319, TESTI4019657, TESTI4021482, TESTI4024387, TESTI4025268, TESTI4031745, THYMU2004139, THYMU2031249, UTERU2008040, UTERU3000738

The clones predicted to belong to the category of transcription-related protein are the following 38 clones.

BRACE2030326, BRACE3001002, BRACE3045033, BRHIP3025161, BRSSN2014299, BRTHA2014792, BRTHA3001721, CTONG2025516, FEBRA2007544, FEBRA2007801, HEART1000074, IMR322000127, IMR322000917, NT2NE2006531, NT2RI2006686, NT2RI3009158, OCBBF2020838, OCBBF2036743, PEBLM2002887, SKNMC2007504, SPLEN2012624, TESTI2026505, TESTI2040018, TESTI2044796, TESTI2050987, TESTI4001176, TESTI4007810, TESTI4014175, TESTI4017543, TESTI4026524, TESTI4036909, THYMU2006420, THYMU2037233, THYMU3004866, TRACH3000558, TUTER2000425, UTERU2035328, UTERU3009490

The following 64 clones are also predicted to belong to the category of transcription-related protein.

BRACE2003609, BRACE3001058, BRACE3001113, BRALZ2017844, BRAMY2035070, BRAMY2035449, BRAMY2035718, BRAMY2039341, BRAWH2006207, BRHIP2017553, CERVX2002013, CTONG1000113, CTONG2003348, CTONG2008721, CTONG2020378, CTONG2020411, CTONG2028758, CTONG3004726, DFNES2011192, FCBBF3010361, FEBRA2014122, FEBRA2027609, HCASM2003018, HCHON2004858, HSYRA2005628, MESAN2005303, MESAN2014412, MESAN2015501, NT2RI2008952, NT2RI2018448, NT2RI3001132, OCBBF2008144, OCBBF2009583, OCBBF2011669, OCBBF2019684, OCBBF2032274, OCBBF3000167, SPLEN2004611, SPLEN2016135, SPLEN2016781, SYNOV2021953, SYNOV4002744, TESOP2001796, TESOP2005199, TESOP2006398, TESOP2006865, TESTI2034251, TESTI4000183, TESTI4000214, TESTI4008302, TESTI4015442, TESTI4025494, TESTI4025547, TESTI4028938, TESTI4032112, THYMU2006001, THYMU2028739, TRACH2007483, TRACH3000134, TRACH3003832, TUTER2000057, UTERU2033577, UTERU3001053, TESTI4038779

The clones predicted to belong to the category of disease-related protein are the following 342 clones.

3NB692002806, ADIPS2000088, BLADE2005036, BRACE2005457, BRACE2008594, BRACE2014306, BRACE2016981, BRACE2018762, BRACE2035381, BRACE2038551, BRACE2039249, BRACE2045300, BRACE3000840, BRACE3001002, BRACE3001391, BRACE3001754, BRACE3002508, BRACE3003595, BRACE3004058, BRACE3004150, BRACE3004772, BRACE3008137, BRACE3008384, BRACE3009708, BRACE3010397, BRACE3011271, BRACE3011421, BRACE3014807, BRACE3015027, BRACE3015521, BRACE3018963, BRACE3020594, BRACE3027326, BRALZ2017359, BRAMY2005052, BRAMY2038904, BRAMY2047751, BRAMY3000213, BRAMY3005091, BRAMY3007609, BRAMY4000095, BRAMY4000229, BRAMY4000277, BRAWH2001395, BRAWH2002560, BRAWH2010000, BRAWH2010536, BRAWH2014414, BRAWH3000100, BRAWH3000491, BRAWH3001326, BRAWH3002574, BRAWH3005912, BRAWH3008341, BRCAN2002562, BRCAN2002856, BRCAN2002948, BRCAN2003746, BRCAN2006063, BRCAN2009203, BRCAN2014602, BRCAN2016619, BRCAN2017442, BRCAN2024451, BRCOC2001505, BRCOC2003213, BRHIP2000819, BRHIP2001805, BRHIP2009414, BRHIP2024165, BRHIP2026288, BRHIP3000339, BRHIP3008405, BRHIP3009448, BRHIP3027137, BRHIP3027854, BRSSN2000684, BRSSN2004719, BRSSN2014424, BRSTN2001613, BRSTN2004987, BRSTN2008418, BRTHA2002608, BRTHA2003110, BRTHA2007122, BRTHA2007603, BRTHA2008527, BRTHA2012980, BRTHA2014792, BRTHA3001721, BRTHA3002427, BRTHA3003074, BRTHA3003449, BRTHA3008778, BRTHA3009037, BRTHA3009090, BRTHA3015815, BRTHA3016917, BRTHA3017848, COLON2000568, COLON2002520, CTONG1000341, CTONG2000042, CTONG2009423, CTONG2010803, CTONG2017500, CTONG2023021, CTONG2025496, CTONG2025516, CTONG3000084, CTONG3002412, CTONG3008639, D3OST2002182, D3OST2002648, DFNES2001108, FCBBF3009888, FEBRA2007708, FEBRA2008468, FEBRA2024744, HCASM2001301, HCASM2007737, HCHON2001712, HCHON2002676, HCHON2003532, HCHON2004007, HCHON2004531, HCHON2008112, HCHON2008444, HEART1000010, HEART1000139, HEART2001680, HEART2010495, HLUNG2002465, HSYRA2005496, IMR322000127, IMR322001380, IMR322006495, KIDNE2001847, KIDNE2012945, NESOP2001694, NOVAR2001108, NT2NE2003252, NT2NE2006531, NT2NE2006909, NT2RI2006686, NT2RI2025909, NT2RI3001515, NT2RI3006171, NT2RI3006340, NT2RI3006673, NT2RI3007757, NT2RI3008652, NT2RP7000359, NT2RP7005118, NT2RP7005529, NT2RP7010599, NTONG2000413, OCBBF2006058, OCBBF2020801, OCBBF2021788, OCBBF2031167, OCBBF2033869, OCBBF2036743, OCBBF2037068, OCBBF2037340, OCBBF3003320, PEBLM2000170, PEBLM2002887, PERIC2003720, PERIC2007914, PERIC2008385, PERIC2009086, PLACE5000282, PLACE6019385, PROST1000184, PROST2003428, PROST2016462, PROST2017367, PROST2018090, PROST2018511, PUAEN2002489, PUAEN2009795, SKNMC2007504, SMINT2010076, SPLEN2002467, SPLEN2006122, SPLEN2011422, SPLEN2012624, SPLEN2021701, SPLEN2031547, SPLEN2033098, SPLEN2036326, SPLEN2036821, SPLEN2036932, SYNOV2005817, SYNOV2012326, SYNOV2014400, SYNOV2021320, SYNOV3000231, SYNOV3000302, SYNOV4002883, SYNOV4004741, SYNOV4007360, SYNOV4007521, SYNOV4007553, SYNOV4007671, SYNOV4008440, TBAES2001229, TBAES2001258, TESOP2004114, TESOP2005485, TESOP2009121, TESTI1000257, TESTI1000319, TESTI2000644, TESTI2002618, TESTI2005610, TESTI2024567, TESTI2026505, TESTI2050987, TESTI2051867, TESTI2053399, TESTI2053621, TESTI4000014, TESTI4000079, TESTI4000288, TESTI4000349, TESTI4000724, TESTI4001148, TESTI4001176, TESTI4001527, TESTI4001561, TESTI4002491, TESTI4006420, TESTI4006819, TESTI4007163, TESTI4007778, TESTI4007810, TESTI4008050, TESTI4008429, TESTI4009160, TESTI4009457, TESTI4009881, TESTI4010851, TESTI4011745, TESTI4011956, TESTI4012406, TESTI4012448, TESTI4012505, TESTI4012679, TESTI4013369, TESTI4013924, TESTI4014175, TESTI4016110, TESTI4016822, TESTI4016925, TESTI4017901, TESTI4018835, TESTI4018881, TESTI4018886, TESTI4020092, TESTI4021478, TESTI4022873, TESTI4023546, TESTI4026524, TESTI4027557, TESTI4028059, TESTI4028429, TESTI4028880, TESTI4030069, TESTI4034632, TESTI4034912, TESTI4035063, TESTI4035498, TESTI4036909, TESTI4037156, TESTI4040363, THYMU1000496, THYMU2005303, THYMU2008725, THYMU2019210, THYMU2027497, THYMU2027695, THYMU2027734, THYMU2031046, THYMU2033104, THYMU2035319, THYMU2037233, THYMU2041015, THYMU3001083, THYMU3001234, THYMU3001379, THYMU3003309, THYMU3004835, THYMU3006118, THYMU3007137, THYMU3008436, TKIDN2000701, TKIDN2006852, TLIVE2001327, TRACH2001549, TRACH2007059, TRACH2022425, TRACH2022649, TRACH3000558, TRACH3002168, TRACH3004721, TRACH3004786, TRACH3005549, TRACH3007479, TRACH3008629, TRACH3009455, TRACH3035526, TSTOM2000442, TUTER2000904, UTERU1000337, UTERU2005621, UTERU2007724, UTERU2017762, UTERU2019491, UTERU2019706, UTERU2025025, UTERU2026090, UTERU2027591, UTERU2035328, UTERU3000645, UTERU3000828, UTERU3000899, UTERU3001240, UTERU3001572, UTERU3001585, UTERU3001652, UTERU3003116, UTERU3003135, UTERU3005907, UTERU3007640, UTERU3008671, UTERU3009490, UTERU3009690, UTERU3009979, UTERU3015500, UTERU3016789

The following 84 clones are also predicted to belong to the category of disease-related protein.

BRACE3001113, BRACE3010076, BRAMY2039341, BRAMY3004800, BRAWH3009017, BRCAN2002473, BRCAN2002854, BRCAN2003070, BRHIP2005271, BRHIP2017553, BRHIP2026877, BRHIP3000240, BRHIP3008314, BRHIP3026052, BRSTN2013354, BRTHA2016318, BRTHA2017972, BRTHA3003000, CERVX2002013, CTONG1000113, CTONG2008721, CTONG2020411, CTONG3004550, FCBBF1000509, FEBRA2008692, HCASM2008536, HCHON2004858, HEART2009680, HLUNG2015548, HSYRA2005628, IMR322008651, IMR322013396, MESAN2001770, NT2RI2009583, NT2RI3007443, OCBBF2003327, OCBBF2009583, OCBBF2011669, OCBBF2024284, OCBBF2032274, OCBBF3000167, OCBBF3002654, PLACE7000502, PROST2000452, PROST2009320, SPLEN2004611, STOMA2003158, SYNOV1000256, SYNOV4002744, SYNOV4003981, TBAES2000932, TESOP2000390, TESOP2001796, TESOP2005199, TESTI2015626, TESTI2025924, TESTI2026647, TESTI2039060, TESTI4000183, TESTI4006473, TESTI4011070, TESTI4017714, TESTI4019657, TESTI4021482, TESTI4024387, TESTI4025494, TESTI4025547, TESTI4028938, TESTI4031745, TESTI4032112, THYMU2004284, THYMU2028739, THYMU2031139, THYMU2031249, THYMU2035710, THYMU3000269, TLIVE2001684, TLIVE2002046, TRACH2024408, TRACH3003683, UTERU2021820, UTERU2032279, UTERU2033577, UTERU3000738

In particular, hit data of the following 338 clones for Swiss-Prot, or GenBank, UniGene, nr, or RefSeq corresponded to genes or proteins which had been deposited in the Online Mendelian Inheritance in Man (OMIM), which is the human gene and disease database (the OMIM Number is shown in the parenthesis after the Clone Name).

3NB692002806 (261630), ADIPS2000088 (147120), BLADE2005036 (114850), BRACE2005457 (274600; 603545; 600791), BRACE2008594 (601959), BRACE2014306 (193002), BRACE2016981 (602701), BRACE2018762 (604800), BRACE2035381 (606088), BRACE2038551 (601961),

BRACE2039249 (602273), BRACE2045300 (601442), BRACE3000840 (600355), BRACE3001002 (300236), BRACE3001391 (601313; 173900), BRACE3001754 (185641), BRACE3002508 (606417), BRACE3003595 (602941), BRACE3004058 (250800), BRACE3004150 (601035), BRACE3004772 (603143), BRACE3008137 (602187), BRACE3008384 (603264), BRACE3009708 (182340), BRACE3010397 (602187), BRACE3011271 (602187), BRACE3011421 (602187), BRACE3014807 (605784), BRACE3015027 (602187), BRACE3015521 (605888), BRACE3018963 (605744), BRACE3020594 (400023), BRACE3027326 (602187), BRALZ2017359 (604331), BRAMY2005052 (602621), BRAMY2038904 (605671), BRAMY2047751 (602512), BRAMY3000213 (605448), BRAMY3005091 (600286), BRAMY3007609 (300315), BRAMY4000095 (602187), BRAMY4000229 (602159), BRAMY4000277 (602187), BRAWH2001395 (159430), BRAWH2002560 (602865), BRAWH2010000 (602581), BRAWH2010536 (604010), BRAWH2014414 (603006), BRAWH3000100 (601403), BRAWH3000491 (602187), BRAWH3001326 (602187), BRAWH3002574 (602187), BRAWH3005912 (602187), BRAWH3008341 (602187), BRCAN2002562 (602187), BRCAN2002856 (602712), BRCAN2002948 (603534), BRCAN2003746 (311870), BRCAN2006063 (603196; 601369), BRCAN2009203 (603143), BRCAN2014602 (601441), BRCAN2016619 (602187), BRCAN2017442 (604455), BRCAN2024451 (602513), BRCOC2001505 (159430), BRCOC2003213 (602187), BRHIP2000819 (605000), BRHIP2001805 (603219), BRHIP2009414 (602187), BRHIP2024165 (604402), BRHIP2026288 (602187), BRHIP3000339 (159430), BRHIP3008405 (602187), BRHIP3009448 (602187), BRHIP3027137 (600249), BRHIP3027854 (601060), BRSSN2000684 (603505), BRSSN2004719 (600560), BRSSN2014424 (606105), BRSTN2001613 (164020), BRSTN2004987 (604733), BRSTN2008418 (602187), BRTHA2002608 (600463), BRTHA2003110 (602187), BRTHA2007122 (106410), BRTHA2007603 (605846), BRTHA2008527 (152790; 176410), BRTHA2012980 (300119), BRTHA2014792 (601674), BRTHA3001721 (604902),

BRTHA3002427 (602187), BRTHA3003074 (605367), BRTHA3003449 (160745), BRTHA3008778 (602187), BRTHA3009037 (602187), BRTHA3009090 (603197), BRTHA3015815 (600902), BRTHA3016917 (604137), BRTHA3017848 (603377; 212140), COLON2000568 (147000), COLON2002520 (602187), CTONG1000341 (188040), CTONG2000042 (103950), CTONG2009423 (182137), CTONG2010803 (602189), CTONG2023021 (602498), CTONG2025496 (103950), CTONG2025516 (601679), CTONG3000084 (600888), CTONG3002412 (601403), CTONG3008639 (601797), D3OST2002182 (603590), D3OST2002648 (603071), DFNES2001108 (603560), FCBBF3009888 (602470), FEBRA2007708 (126650; 214700), FEBRA2008468 (278000), HCASM2001301 (602399), HCASM2007737 (601504), HCHON2001712 (109190),

HCHON2002676 (252800), HCHON2003532 (172490), HCHON2004007 (605866), HCHON2004531 (602187), HCHON2008112 (605837), HCHON2008444 (602187), HEART1000010 (602187), HEART1000139 (191045; 115195), HEART2001680 (146900), HEART2010495 (157132), HLUNG2002465 (605216), HSYRA2005496 (131195; 187300), IMR322000127 (604077), IMR322001380 (605652), IMR322006495 (605607), KIDNE2012945 (600270), NOVAR2001108 (147120), NT2NE2003252 (602913), NT2NE2006531 (602277), NT2NE2006909 (602187),

NT2RI2006686 (602700), NT2RI2025909 (212138), NT2RI3001515 (300362), NT2RI3006171 (114890), NT2RI3006340 (602187), NT2RI3006673 (602187), NT2RI3007757 (605396), NT2RI3008652 (602654), NT2RP7000359 (603271), NT2RP7005118 (603379), NT2RP7005529 (600888), NT2RP7010599 (603684), NTONG2000413 (602262), OCBBF2006058 (604773), OCBBF2020801 (602187), OCBBF2021788 (602597), OCBBF2031167 (603709), OCBBF2033869 (600270), OCBBF2036743 (604075), OCBBF2037068 (602187), OCBBF2037340 (602187), OCBBF3003320 (605868), PEBLM2000170 (602187), PEBLM2002887 (602187), PERIC2003720 (600381), PERIC2007914 (400009), PERIC2008385 (604455), PERIC2009086 (600134; 605158), PLACE5000282 (130160), PLACE6019385 (602448), PROST1000184 (192321), PROST2003428 (602187), PROST2016462 (602187), PROST2017367 (600585), PROST2018090 (312610), PROST2018511 (602187), PUAEN2002489 (604658), PUAEN2009795 (601456), SKNMC2007504 (602187), SMINT2010076 (146900), SPLEN2002467 (605652), SPLEN2006122 (604739), SPLEN2011422 (114213), SPLEN2012624 (602187), SPLEN2021701 (142800), SPLEN2031547 (602187), SPLEN2033098 (602746), SPLEN2036326 (602101), SPLEN2036821 (212138), SPLEN2036932 (605577), SYNOV2005817 (123889), SYNOV2012326 (604336), SYNOV2014400 (135820), SYNOV2021320 (602104), SYNOV3000231 (147100), SYNOV3000302 (147100), SYNOV4002883 (602187), SYNOV4004741 (602187), SYNOV4007360 (602187), SYNOV4007521 (605830), SYNOV4007553 (603028), SYNOV4007671 (602187), SYNOV4008440 (602187), TBAES2001229 (602187), TBAES2001258 (142440), TESOP2004114 (601865), TESOP2005485 (147170), TESOP2009121 (117143), TESTI1000257 (138170), TESTI1000319 (602187), TESTI2000644 (60-1392), TESTI2002618 (601533), TESTI2005610 (601040), TESTI2024567 (601116), TESTI2026505 (305400), TESTI2050987 (605968), TESTI2051867 (180479), TESTI2053399 (605819), TESTI2053621 (600364; 602093), TESTI4000014 (602187), TESTI4000079 (603560), TESTI4000288 (602187), TESTI4000349 (604506), TESTI4000724 (603878), TESTI4001148 (602187), TESTI4001176 (601430), TESTI4001527 (602187), TESTI4001561 (602187), TESTI4002491 (602187), TESTI4006420 (605612), TESTI4006819 (602187), TESTI4007163 (602187), TESTI4007778 (602187), TESTI4007810 (600940), TESTI4008050 (602187), TESTI4008429 (602187), TESTI4009160 (602187), TESTI4009457 (606185), TESTI4009881 (602187), TESTI4010851 (602187), TESTI4011745 (602187), TESTI4011956 (602187), TESTI4012406 (602187), TESTI4012448 (185261), TESTI4012505 (602143), TESTI4012679 (601933), TESTI4013369 (602187), TESTI4013924 (602187), TESTI4014175 (602187), TESTI4016110 (602187), TESTI4016822 (601792), TESTI4016925 (602187), TESTI4017901 (104221), TESTI4018835 (602187), TESTI4018881 (605070), TESTI4018886 (602187), TESTI4020092 (156225), TESTI4021478 (605868), TESTI4022873 (602187), TESTI4023546 (602187), TESTI4026524 (603277), TESTI4027557 (602187), TESTI4028059 (232800; 171850), TESTI4028429 (602187), TESTI4028880 (138170), TESTI4030069 (604603), TESTI4034632 (606251), TESTI4034912 (602187), TESTI4035063 (602187), TESTI4035498 (602187), TESTI4036909 (602187), TESTI4037156 (606026), TESTI4040363 (185641), THYMU1000496 (603060), THYMU2005303 (186910), THYMU2008725 (176882), THYMU2019210 (142830), THYMU2027497 (182139), THYMU2027695 (147100), THYMU2027734 (145505), THYMU2031046 (604207), THYMU2033104 (605349), THYMU2035319 (604739), THYMU2037233 (605121), THYMU2041015 (602187), THYMU3001083 (602187), THYMU3001234 (602187), THYMU3001379 (602187), THYMU3003309 (300359), THYMU3004835 (602187), THYMU3006118 (603708), THYMU3007137 (602187), THYMU3008436 (602187), TKIDN2000701 (600465), TKIDN2006852 (603602), TLIVE2001327 (601403), TRACH2001549 (603197), TRACH2007059 (602187), TRACH2022425 (146900), TRACH2022649 (147100), TRACH3000558 (600140), TRACH3002168 (155735), TRACH3004721 (602187), TRACH3004786 (602187), TRACH3005549 (602187), TRACH3007479 (602308), TRACH3008629 (600976), TRACH3009455 (171833), TRACH3035526 (147000), TSTOM2000442 (147100), TUTER2000904 (602187), UTERU1000337 (602187), UTERU2005621 (603505), UTERU2007724 (602373), UTERU2017762 (601053), UTERU2019491 (603762), UTERU2019706 (600114), UTERU2025025 (191315; 164970; 256000), UTERU2026090 (605497), UTERU2027591 (600150),

UTERU2035328 (605409), UTERU3000645 (602909), UTERU3000828 (602187), UTERU3000899 (603062), UTERU3001240 (602187), UTERU3001572 (602187), UTERU3001585 (602187), UTERU3001652 (602715), UTERU3003116 (602187), UTERU3003135 (602187), UTERU3005907 (190196), UTERU3007640 (603215), UTERU3008671 (182120), UTERU3009490 (604585), UTERU3009690 (104221), UTERU3009979 (600441), UTERU3015500 (606667), UTERU3016789 (602104)

Additionally, hit data of the following 84 clones for Swiss-Prot, or nr or RefSeq corresponded to genes or proteins which had been deposited in the Online Mendelian Inheritance in Man (OMIM), which is the human gene and disease database (the OMIM Number is shown in the parenthesis after the Clone Name).

BRACE3001113 (603971), BRACE3010076 (142695), BRAMY2039341 (604077), BRAMY3004800 (602187), BRAWH3009017 (602187), BRCAN2002473 (602187), BRCAN2002854 (602895), BRCAN2003070 (605574), BRHIP2005271 (600267), BRHIP2017553 (602187), BRHIP2026877 (600341), BRHIP3000240 (601142), BRHIP3008314 (604480), BRHIP3026052 (601645), BRSTN2013354 (602187), BRTHA2016318 (605442), BRTHA2017972 (602932), BRTHA3003000 (605276), CERVX2002013 (602903), CTONG1000113 (602277), CTONG2008721 (605317), CTONG2020411 (601930), CTONG3004550 (605611), FCBBF1000509 (601933), FEBRA2008692 (604034), HCASM2008536 (194360), HCHON2004858 (602187), HEART2009680 (601970), HLUNG2015548 (146690), HSYRA2005628 (602187), IMR322008651 (179617), IMR322013396 (600053), MESAN2001770 (600495), NT2RI2009583 (605949), NT2RI3007443 (602448), OCBBF2003327 (605008), OCBBF2009583 (602277), OCBBF2011669 (602187), OCBBF2024284 (176981), OCBBF2032274 (603975), OCBBF3000167 (194558), OCBBF3002654 (601893), PLACE7000502 (164951), PROST2000452 (602060), PROST2009320 (605903), SPLEN2004611 (602228), STOMA2003158 (602244), SYNOV1000256 (606021), SYNOV4002744 (602187), SYNOV4003981 (604283), TBAES2000932 (606212), TESOP2000390 (602187), TESOP2001796 (602187), TESOP2005199 (194531), TESTI2015626 (601249), TESTI2025924 (600863), TESTI2026647 (601235), TESTI2039060 (154360), TESTI4000183 (601276), TESTI4006473 (602187), TESTI4011070 (602187), TESTI4017714 (602187), TESTI4019657 (602052), TESTI4021482 (164730), TESTI4024387 (602187), TESTI4025494 (602187), TESTI4025547 (605308), TESTI4028938 (603899), TESTI4031745 (602448), TESTI4032112 (603246), THYMU2004284 (314370), THYMU2028739 (604191), THYMU2031139 (605009), THYMU2031249 (311550), THYMU2035710 (601890), THYMU3000269 (600857), TLIVE2001684 (120700), TLIVE2002046 (125270), TRACH2024408 (106410), TRACH3003683 (150205), UTERU2021820 (126141), UTERU2032279 (600942), UTERU2033577 (603397), UTERU3000738 (602187)

The clones predicted to belong to the category of enzyme and/or metabolism-related protein are the following 171 clones.

3NB692002806, ASTRO2002842, BLADE2005036, BRACE2008594, BRACE2030341, BRACE2035381, BRACE2038551, BRACE2039249, BRACE2041200, BRACE2045772, BRACE3004058, BRACE3009708, BRACE3011421, BRACE3016884, BRACE3024073, BRACE3025630, BRAMY2033267, BRAMY2039872, BRAMY3002803, BRAMY3004919, BRAMY3005091, BRAMY3005932, BRAMY4000095, BRAWH3002574, BRAWH3008341, BRCAN2003703, BRCAN2003746, BRCAN2009432, BRCAN2014602, BRCAN2017442, BRCAN2028355, BRCOC2003213, BRHIP2024165, BRHIP3008405, BRHIP3027137, BRHIP3027854, BRSTN2000872, BRSTN2004863, BRSTN2004987, BRSTN2008418, BRTHA2002608, BRTHA2009311, BRTHA2015406, BRTHA2016496, BRTHA3008778, BRTHA3009090, BRTHA3015815, BRTHA3016917, CTONG2004062, CTONG2006798, CTONG2013178, CTONG2028124, CTONG3009028, D3OST2002182, DFNES2001108, DFNES2005266, FCBBF3013307, FCBBF3023895, FEBRA2008468, FEBRA2026984, HCASM2001301, HCHON2002676, HCHON2003532, HCHON2004007, HEART2006131, HEART2010492, HHDPC1000118, HLUNG2011298, HLUNG2013204, HSYRA2008714, KIDNE2001361, KIDNE2006580, NT2NE2003252, NT2NE2006909, NT2RI2004618, NT2RI2025909, NT2RI3006673, NT2RI3007978, NT2RI3008974, NT2RP7000359, NT2RP7004027, NT2RP7010599, NT2RP7014005, NTONG2000413, NTONG2008672, OCBBF2006005, OCBBF2006058, OCBBF2006151, OCBBF2019823, OCBBF2025527, OCBBF2030354, OCBBF2031167, OCBBF3003320, PEBLM2005183, PERIC2000889, PERIC2008385, PLACE6019385, PLACE7008431, PROST2017367, PUAEN2007044, PUAEN2009655, PUAEN2009852, SKNMC2006998, SKNMC2007504, SMINT1000192, SPLEN2010912, SYNOV2012326, SYNOV4002883, TBAES2001258, TESOP2000801, TESOP2004114, TESTI2005610, TESTI2005739, TESTI2016046, TESTI4000079, TESTI4000209, TESTI4000288, TESTI4000349, TESTI4001176, TESTI4001527, TESTI4001561, TESTI4002552, TESTI4006148, TESTI4006819, TESTI4007810, TESTI4008429, TESTI4010851, TESTI4012406, TESTI4012448, TESTI4013369, TESTI4013817, TESTI4014175, TESTI4016822, TESTI4018152, TESTI4018835, TESTI4019566, TESTI4021478, TESTI4022716, TESTI4023546, TESTI4026510, TESTI4026524, TESTI4028059, TESTI4029836, TESTI4034632, TESTI4036909, TESTI4046819, THYMU2008725, THYMU2027734, THYMU2031046, THYMU2031258, THYMU3001234, THYMU3003212, THYMU3004157, THYMU3004835, THYMU3006118, THYMU3008436, TKIDN2006852, TLIVE2002336, TRACH2001549, TRACH2009310, TRACH3007479, TRACH3036193, UTERU1000337, UTERU2019491, UTERU2025025, UTERU2026203, UTERU3000665, UTERU3001240, UTERU3001585, UTERU3003116, UTERU3005907

The following 59 clones are also predicted to belong to the category of enzyme and/or metabolism-related protein.

BRACE2039823, BRACE3010076, BRAMY2038516, BRAWH1000369, BRCAN2003070, BRHIP2005271, BRHIP2012360, BRHIP2026877, BRHIP3008314, BRTHA2013610, BRTHA2017364, BRTHA2017972, BRTHA2018011, BRTHA2018443, BRTHA3003000, CTONG2016942, FCBBF1000509, FEBRA2008692, HCASM2003099, HLUNG2015548, MESAN2005303, NT2RI3000174, NT2RI3007443, NT2RP7008435, NTONG2008093, OCBBF2003327, OCBBF2034637, OCBBF3002654, PROST2000452, SPLEN2039311, SPLEN2039379, STOMA2003158, TESOP2000390, TESTI2015626, TESTI2025924, TESTI2026647, TESTI2032643, TESTI2036288, TESTI2039060, TESTI4006473, TESTI4011070, TESTI4014801, TESTI4017714, TESTI4019657, TESTI4021482, TESTI4031745, THYMU2004139, THYMU2004284, THYMU2031139, THYMU2031249, THYMU2040925, THYMU3000269, TLIVE2002046, TLIVE2007607, TRACH2024559, TRACH3003683, TRACH3007866, UTERU2021820, UTERU3000738

The clones predicted to belong to the category of cell division and/or cell proliferation-related protein are the following 42 clones.

BLADE2002782, BRACE2042550, BRACE2043248, BRACE3000840, BRALZ2017359, BRAMY2038484, BRAMY2046989, BRAWH2010536, BRAWH2014954, BRAWH3000100, BRHIP2000819, BRHIP2001927, BRHIP2009414, BRSSN2000684, CTONG3002412, CTONG3008258, CTONG3008639, FCBBF3002163, HCASM2001301, IMR322006495, NT2RI2006686, OCBBF2021020, OCBBF2037068, OCBBF3004314, PLACE5000282, PLACE6019385, PLACE7002641, PUAEN2006328, SPLEN2033098, TESOP2009121, TESTI1000545, TESTI2003573, TESTI2005610, TESTI4007810, TESTI4017901, THYMU2034374, THYMU2039315, TLIVE2001327, TRACH2025507, UTERU2005621, UTERU3009690, UTERU3009979

The following ten clones are also predicted to belong to the category of cell division and/or cell proliferation-related protein.

BRCAN2003070, BRTHA3003000, NT2RI3007443, PLACE7000502, SPLEN2004611, STOMA2003158, SYNOV4003981, TESTI4031745, THYMU2004139, THYMU2035078

The clones predicted to belong to the category of cytoskeleton-related protein are the following 55 clones.

ASTRO1000009, BLADE2004089, BRACE2026836, BRACE2045300, BRACE3006872, BRAMY3008466, BRAWH2001395, BRAWH2005315, BRAWH3002600, BRCOC2001505, BRHIP2000819, BRHIP3000339, BRHIP3008405, BRTHA2007122, BRTHA3003449, COLON2002520, CORDB2000541, FCBBF3021940, HCHON2001577, HEART1000139, HEART2010495, NT2RI3006340, NT2RP7000359, NTONG2005277, OCBBF2007068, OCBBF3003592, PERIC2000889, PLACE5000282, PROST1000559, SKMUS2006394, SPLEN2011422, SPLEN2015679, TESTI2049857, TESTI4000288, TESTI4001148, TESTI4007778, TESTI4009160, TESTI4009881, TESTI4011956, TESTI4013924, TESTI4016925, TESTI4018886, TESTI4022873, TESTI4034912, TESTI4035063, TESTI4037727, THYMU1000496, THYMU2035735, THYMU3001083, THYMU3001234, TKIDN2000701, UTERU2007724, UTERU2008347, UTERU2035745, UTERU3003178

The following six clones are also predicted to belong to the category of cytoskeleton-related protein.

HLUNG2015418, SPLEN2030847, SPLEN2036702, TESTI4025268, TESTI4026207, TRACH2024408

The clones predicted to belong to the category of nuclear protein and/or RNA synthesis-related protein are the following 42 clones.

BLADE2007958, BRACE2010489, BRACE2045300, BRACE3004150, BRACE3005430, BRACE3011421, BRAMY2046989, BRAMY3005932, BRCAN2002562, BRHIP2021615, BRSTN2001613, BRSTN2004987, COLON2000470, CTONG3009028, FCBBF3013307, HCHON2004531, IMR322006495, OCBBF2020801, PEBLM2005183, PUAEN2007044, SKNMC1000124, SMINT1000192, SPLEN2006122, SPLEN2010912, TESOP2009121, TESTI4009374, TESTI4009457, TESTI4013830, TESTI4019566, TESTI4022716, THYMU2033104, THYMU2035319, THYMU2038301, THYMU2040975, THYMU3001379, TRACH3004721, TRACH3036609, UTERU2026025, UTERU3000828, UTERU3001572, UTERU3003135, UTERU3004992

The following 16 clones are also predicted to belong to the category of nuclear protein and/or RNA synthesis-related protein.

BRACE3010076, BRCAN2002854, BRHIP2006617, BRHIP2012360, BRHIP3026052, BRSTN2013354, BRTHA2017364, HCASM2003099, HCASM2008536, IMR322008651, NT2RI3000174, STOMA2003158, TESTI2026647, TESTI4006473, TESTI4021482, THYMU2035078

The clones predicted to belong to the category of protein synthesis and/or transport-related protein are the following 57 clones.

ASTRO2002842, BLADE2005036, BRACE3025630, BRAMY2033003, BRAMY3007609, BRAWH3000491, BRAWH3002574, BRAWH3008341, BRCAN2002856, BRCAN2002948, BRCOC2003213, BRSTN2004987, BRTHA2016496, BRTHA3013884, BRTHA3016917, CTONG2000042, CTONG2013178, CTONG2023512, CTONG2024749, CTONG2025496, CTONG3001370, DFNES2005266, FEBRA2026984, HCASM2007737, HCHON2008444, HEART1000010, KIDNE2000846, NT2NE2006909, NT2RI2011422, NT2RP7004027, NTONG2000413, OCBBF2031167, TBAES2001229, TBAES2001258, TESTI1000319, TESTI2005610, TESTI2051867, TESTI4000209, TESTI4000349, TESTI4001106, TESTI4002491, TESTI4008050, TESTI4010851, TESTI4012406, TESTI4012448, TESTI4013924, TESTI4028429, TESTI4034912, THYMU2009157, TLIVE2008229, TRACH3007479, TRACH3008713, TRACH3036193, UTERU2019940, UTERU3001988, UTERU3003116, UTERU3007419

The following 15 clones are also predicted to belong to the category of protein synthesis and/or transport-related protein.

BRTHA2007060, BRTHA2018011, CTONG2016942, MESAN2001770, MESAN2005303, NT2RP7008435, OCBBF2003327, PROST2000452, TESOP2001796, TESTI4017714, THYMU2004284, THYMU2031139, TRACH2024559, TRACH3007866, UTERU2021820

The clones predicted to belong to the category of cellular defense-related protein are the following three clones.

BRACE3005430, HCHON2004531, TESTI4007810

The following four clones are also predicted to belong to the category of cellular defense-related protein.

BRHIP2012360, FCBBF3027854, HCASM2008536, UTERU2032279

The clones predicted to belong to the category of development and/or differentiation-related protein are the following nine clones.

BRACE3009747, BRTHA2005579, BRTHA3003343, IMR322000917, PEBLM2000170, TESOP2001122, TESOP2001953, TESTI2040018, UTERU3006308

The following five clones are also predicted to belong to the category of development and/or differentiation-related protein.

BRALZ2017844, CTONG2020378, HCHON2004858, OCBBF2019684, THYMU2006001

The clones predicted to belong to the category of DNA-binding and/or RNA-binding protein are the following 55 clones.

3NB692002685, BLADE2007958, BRACE2030326, BRACE2045596, BRACE3001002, BRACE3004150, BRACE3009747, BRACE3045033, BRCAN2002562, BRHIP2021615, BRSSN2014299, BRSTN2001613, BRSTN2004987, BRTHA2014792, BRTHA3001721, BRTHA3003343, CTONG2025516, CTONG3008831, CTONG3009028, FCBBF3013307, FEBRA2007544, FEBRA2007801, HEART1000074, IMR322000127, IMR322000917, NT2NE2006531, NT2RI3009158, OCBBF2020838, OCBBF2036743, PEBLM2002887, PEBLM2005183, SKNMC2007504, SMINT1000192, SPLEN2006122, TBAES2001229, TESTI2014716, TESTI2040018, TESTI2044796, TESTI4009.374, TESTI4012679, TESTI4014175, TESTI4017543, TESTI4026510, TESTI4026524, THYMU2006420, THYMU2035319, THYMU2037233, THYMU2040975, THYMU3004866, TLIVE2008229, TRACH3036609, TUTER2000425, UTERU2026025, UTERU2035328, UTERU3009490

The following 74 clones are also predicted to belong to the category of DNA-binding and/or RNA-binding protein.

BRACE2003609, BRACE3001058, BRACE3001113, BRACE3010076, BRALZ2017844, BRAMY2035070, BRAMY2035449, BRAMY2035718, BRAMY2039341, BRAWH1000369, BRAWH2006207, BRCAN2002854, BRHIP2012360, BRHIP2017553, BRSTN2013354, BRTHA2017364, CERVX2002013, CTONG1000113, CTONG2008721, CTONG2020378, CTONG2020411, CTONG2028758, CTONG3004726, DFNES2011192, FEBRA2014122, FEBRA2027609, HCASM2003018, HCASM2009424, HCHON2004858, HSYRA2005628, IMR322008651, MESAN2001770, MESAN2005303, MESAN2014412, MESAN2015501, NT2RI2008952, NT2RI2018448, NT2RI3000174, NT2RI3001132, OCBBF2008144, OCBBF2009583, OCBBF2011669, OCBBF2019684, OCBBF2032274, OCBBF3000167, SPLEN2004611, SPLEN2016135, SPLEN2016781, SYNOV2021953, SYNOV4002744, TESOP2005199, TESOP2006398, TESOP2006865, TESTI2026647, TESTI2034251, TESTI4000183, TESTI4000214, TESTI4006473, TESTI4008302, TESTI4015442, TESTI4025494, TESTI4025547, TESTI4028938, TESTI4032112, THYMU2006001, THYMU2035078, TRACH2007483, TRACH3000134, TRACH3002561, TRACH3003832, TUTER2000057, UTERU2033577, UTERU3001053, TESTI4038779

The clones predicted to belong to the category of ATP binding and/or GTP-binding protein are the following 68 clones.

BNGH42007788, BRACE2008594, BRACE2047377, BRACE3005430, BRACE3008720, BRACE3009708, BRACE3015521, BRACE3024073, BRAMY4000095, BRCAN2009432, BRCOC2003213, BRHIP3008405, BRSTN2013741, BRTHA3003449, BRTHA3015815, BRTHA3016917, COLON2002520, FEBRA2026984, HCASM2001301, HCHON2004007, HSYRA2008714, KIDNE2001361, KIDNE2001847, NESOP2001694, NT2RI2005166, NT2RP7013795, OCBBF3003320, OCBBF3003592, PEBLM2002594, PERIC2000889, PLACE6019385, SMINT1000192, SPLEN2037194, TESOP2000801, TESTI2006648, TESTI4000288, TESTI4001148, TESTI4001176, TESTI4002552, TESTI4007810, TESTI4008429, TESTI4009160, TESTI4009881, TESTI4011956, TESTI4013817, TESTI4014175, TESTI4016925, TESTI4018208, TESTI4018835, TESTI4019566, TESTI4021478, TESTI4022873, TESTI4026524, TESTI4029836, TESTI4035498, TESTI4036909, TESTI4037727, THYMU1000496, THYMU2033079, THYMU3001083, THYMU3001234, THYMU3001379, TRACH2009310, UTERU2019706, UTERU2025025, UTERU2035745, UTERU3000665, UTERU3000828

The following 24 clones are also predicted to belong to the category of ATP binding and/or GTP-binding protein.

BRHIP2026877, BRTHA2017364, BRTHA2018443, IMR322008651, IMR322013731, NT2RI3007443, NTONG2008093, OCBBF3002654, TESOP2000390, TESOP2007384, TESTI2025924, TESTI2026647, TESTI2049956, TESTI4005317, TESTI4006473, TESTI4021482, TESTI4026207, TESTI4031745, THYMU2004139, THYMU2031249, TRACH2000862, TRACH2024559, TRACH3000420, UTERU3000738

The 119 clones shown below are clones which were unassignable to any of the above-mentioned categories, but have been predicted to have some functions based on homology search using their full-length nucleotide sequences. Clone Name and Definition in the result of homology search, demarcated by a double slash mark (//), are shown below.

ADRGL2009691//Mus musculus D11lgp1 mRNA, complete cds.

ADRGL2009755//Homo sapiens brain and reproductive organ-expressed protein (BRE) mRNA, complete cds.

ASTRO3000177//Drosophila melanogaster BcDNA.GH03694 (BcDNA.GH03694) mRNA, complete cds.

BLADE2008398//Homo sapiens LRR FLI-I interacting protein 2 (LRRFIP2) mRNA, complete cds.

BRACE2006319//Homo sapiens mRNA for Fln29, complete cds.

BRACE2027258//Homo sapiens E2a-Pbx1-associated protein (EB-1) mRNA, partial cds.

BRACE2038329//Rattus norvegicus CBL-B (Cbl-b) mRNA, partial cds.

BRACE2046251//Homo sapiens hucep-10 mRNA for cerebral protein-10, complete cds.

BRACE3003192//latent transforming growth factor beta binding protein 3 [Homo sapiens]

BRACE3007625//espin [Rattus norvegicus]

BRACE3009297//mdgl-1 [Mus musculus]

BRACE3015262//espin [Mus musculus]

BRACE3025457//testis-specific protein TSP-NY [Homo sapiens]

BRALZ2016498//Homo sapiens FKSG76 (FKSG76) mRNA, complete cds.

BRAMY2030109//Homo sapiens hucep-4 mRNA for cerebral protein-4, complete cds.

BRAMY2031317//Mus musculus semaphorin cytoplasmic domain-associated protein 3A (Semcap3) mRNA, complete cds.

BRAMY2047746//nasopharyngeal carcinoma susceptibility protein [Homo sapiens]

BRAMY3001794//Rattus norvegicus Circadian Oscillatory Protein (SCOP) (Scop)

BRAWH2001940//H. sapiens gene from PAC 1026E2, partial.

BRAWH2012162//KE03 protein [Homo sapiens]

BRAWH2016724//MAP2=HMW−MAP2 {alternatively spliced} [rats, brain, mRNA Partial, 267 nt].

BRAWH3002821//synaptotagmin-like 2 [Mus musculus]

BRCAN2002944//Mus musculus huntington yeast partner C (Hypc) mRNA, complete cds.

BRCAN2013660//Arabidopsis thaliana putative protein (F4F15.330) mRNA, complete cds.

BRHIP2002122//Homo sapiens B aggressive lymphoma long isoform (BAL) mRNA, complete cds.

BRHIP2003786//CCA3 [Rattus norvegicus]

BRHIP2004359//ELAC PROTEIN.

BRHIP2007616//plexin 2

BRHIP2029393//COBW-like protein [Homo sapiens]

BRHIP3008313//testis specific ankyrin-like protein 1 [Homo sapiens]

BRSSN2013874//TEMO [Rattus norvegicus]

BRSTN2017771//Homo sapiens putative BTK-binding protein mRNA, complete cds.

BRTHA2012392//Homo sapiens HCDI (HCDI) mRNA, complete cds.

BRTHA3002933//uroplakin 3 [Homo sapiens]

BRTHA3008310//Mus musculus mRNA for iroquois homeobox protein 6 (Irx6 gene).

BRTHA3008520//sporulation-induced transcript 4-associated protein; hypothetical protein FLJ11058 [Homo sapiens]

COLON2001721//GLUT4 vesicle protein [Mus musculus]

CTONG1000467//Mus musculus mRNA for Deltex3, complete cds.

CTONG2020026//Drosophila melanogaster BcDNA.GH09358 (BcDNA.GH09358) mRNA, complete cds.

CTONG3001123//Mus musculus Pax transcription activation domain interacting protein PTIP mRNA, complete cds.

CTONG3002127//granuphilin [Mus musculus]

CTONG3004072//GL002 protein [Homo sapiens]

CTONG3006186//syntaxin binding protein 4 [Mus musculus]

CTONG3008894//Mus musculus SH3-domain binding protein 5

FCBBF1000297//Human protein immuno-reactive with anti-PTH polyclonal antibodies mRNA, partial cds.

HCHON2000028//Homo sapiens 7h3 protein mRNA, partial cds.

HCHON2000626//X-linked protein STS1769.

HCHON2001217//Homo sapiens cullin CUL4B (CUL4B) mRNA, complete cds.

HEART2006909//Hemolysin C.

HLUNG2011041//basic proline-rich peptide IB-8a—human (fragments)

HLUNG2014288//Mus musculus RP42 mRNA, complete cds.

IMR322006886//Homo sapiens hepatocellular carcinoma-associated antigen 127 (HCA127) mRNA, complete cds.

KIDNE2002252//Drosophila melanogaster BcDNA.GH09358 (BcDNA.GH09358) mRNA, complete cds.

KIDNE2011532//similar to melanoma-associated chondroitin sulfate proteoglycan 4

NT2RI2012990//76.5 KDA PROTEIN C21ORF13.

NT2RI2025957//LU1 protein [Homo sapiens]

NT2RI3006284//Homo sapiens chorea-acanthocytosis (CHAC) mRNA, complete cds.

NT2RI3008697//erythroblast macrophage protein [Mus musculus]

NT2RP8000296//similar to Kelch proteins

NTONG2007517//RING CANAL PROTEIN (KELCH PROTEIN).

OCBBF2002124//p40 [Homo sapiens]

OCBBF2007610//PSD-95/SAP90-associated protein-4 [Rattus norvegicus].

OCBBF2021323//Mus musculus GTRGEO22 (Gtrgeo22) mRNA, complete cds.

OCBBF2028173//JM11 protein [Homo sapiens]

PEBLM2001465//diphthamide biosynthesis; Dph5p [Saccharomyces cerevisiae]

PERIC2004028//Mus musculus erythroblast macrophage protein EMP mRNA, complete cds.

PLACE7006051//cytoplasmic dynein heavy chain 2 [Rattus norvegicus]

PROST2008993//Mus musculus Pax transcription activation domain interacting protein PTIP mRNA, complete cds.

PUAEN2003079//nasopharyngeal carcinoma susceptibility protein [Homo sapiens]

SPLEN2002147//Halocynthia roretzi mRNA for HrPET-3, complete cds.

SPLEN2032154//NDRG1 PROTEIN (DIFFERENTIATION-RELATED GENE 1 PROTEIN) (DRG1) (REDUCING AGENTS AND TUNICAMYCIN-RESPONSIVE PROTEIN) (RTP) (NICKEL-SPECIFIC INDUCTION PROTEIN CAP43).

SYNOV2005216//Homo sapiens laryngeal carcinoma related protein 1 mRNA, complete cds.

SYNOV2007965//Homo sapiens mRNA for H-1(3)mbt-like protein, alternative variant a.

SYNOV4000706//B cell phosphoinositide 3-kinase adaptor [Mus musculus]

TBAES2004055//NY-REN-50 antigen

TESOP2001605//Homo sapiens laryngeal carcinoma related protein 1 mRNA, complete cds.

TESOP2005285//Homo sapiens partial mRNA for chr2 synaptotagmin (CHR2SYT gene).

TESTI2004215//Maackia amurensis early nodulin (ENOD2) mRNA, partial cds.

TESTI2009477//TRICHOHYALIN.

TESTI2034520//Rattus norvegicus SMC (segregation of mitotic chromosomes 1)-like 1 (yeast) (Smc111), mRNA

TESTI2052693//brk kinase substrate [Homo sapiens].

TESTI4006079//MUF1 protein; likely ortholog of mouse MUF1; elongin BC-interacting leucine-rich repeat protein [Homo sapiens]

TESTI4006393//neural specific sr protein NSSR 2 [Mus musculus]

TESTI4006546//colon cancer antigen NY-CO-45 [Homo sapiens].

TESTI4006802//mesothelin; megakaryocyte potentiating factor [Mus musculus]

TESTI4008018//DAZ associated protein 2; KIAA0058 gene product [Homo sapiens]

TESTI4009286//Homo sapiens HOTTL protein mRNA, complete cds

TESTI4009563//testis specific ankyrin-like protein 1 [Homo sapiens]

TESTI4010831//yeast Sec31p homolog; ABP125 [Homo sapiens]

TESTI4011484//Sec23-interacting protein p125 [Homo sapiens]

TESTI4014818//AD-012 protein [Homo sapiens]

TESTI4014924//selective hybridizing clone [Mus musculus]

TESTI4019140//Mi-2 histone deacetylase complex protein 66 [Xenopus laevis]

TESTI4019843//Rattus norvegicus huntingtin-associated protein interacting protein (duo) (Hapip), mRNA.

TESTI4023762//Trichohyalin.

TESTI4025920//B29 protein [Homo sapiens]

TESTI4039659//DnaJ homolog subfamily B member 8 (mDJ6).

TESTI4044186//leucine-rich, glioma inactivated 1 [Mus musculus]

THYMU2011736//latent transforming growth factor beta binding protein 3

THYMU2032825//Mus musculus mRNA for Drctnnb1a, complete cds.

THYMU2038369//Mus musculus GTRGEO22 (Gtrgeo22) mRNA, complete cds.

THYMU3001991//ART-4 protein [Homo sapiens]

THYMU3006172//membrane bound C2 domain containing protein [Rattus norvegicus]

TLIVE2003225//CUB and Sushi multiple domains 1 [Homo sapiens]

TLIVE2004320//Homo sapiens PC2-glutamine-rich-associated protein (PCQAP) mRNA, complete cds.

TOVAR2002247//Homo sapiens partial partial mRNA for NICE-4 protein, clone 3114f17.

TRACH2023299//growth factor receptor bound protein 2-associated protein 2 [Mus musculus]

TRACH3000926//cardiac morphogenesis [Mus musculus]

TRACH3001427//p47 [Homo sapiens]

TRACH3006412//Homo sapiens COP9 constitutive photomorphogenic homolog subunit 7B

TRACH3034731//Ras association (RalGDS/AF-6) domain family 2

TUTER2002729//D6MM5E protein [Mus musculus]

UTERU1000031//G. gallus mRNA for tom-1B protein.

UTERU2006115//ALPHA-ADAPTIN A (CLATHRIN ASSEMBLY PROTEIN COMPLEX 2 ALPHA-A LARGE CHAIN) (100 KDA COATED VESICLE PROTEIN A). (PLASMA MEMBRANE ADAPTOR HA2/AP2 ADAPTIN ALPHA A SUBUNIT).

UTERU2031268//NY-REN-25 antigen [Homo sapiens].

UTERU2035452//NG3 [Homo sapiens]

UTERU3001059//ABC1 protein homolog, mitochondrial precursor.

UTERU3005585//rhophilin-like protein [Homo sapiens]

UTERU3009871//feminization 1 homolog a (C. elegans)

Similarly, the 14 clones shown below are clones which were unassignable to any of the above-mentioned categories, but have been predicted to have some functions based on homology search using their full-length nucleotide sequences. Clone Name and Definition in the result of homology search, demarcated by a double slash mark (//), are shown below.

ADRGL2000042//Homo sapiens CTCL tumor antigen se20-4 mRNA, complete cds.

BRACE3009127//oxysterol binding protein 2; oxysterol binding protein-like 1 [Homo sapiens]

BRACE3021148//DC12 protein [Homo sapiens]

BRAMY2040159//Homo sapiens MRIP-1 mRNA, complete cds.

BRAWH3007441//CAT56 protein [Homo sapiens]

CTONG3001501//Mus musculus glucocorticoid-induced gene 1 mRNA, complete cds.

HCHON2000508//Homo sapiens prostate antigen PARIS-1 mRNA, complete cds.

OCBBF2020048//95 kDa retinoblastoma protein binding protein; KIAA0661 gene product

PERIC2007068//Mus musculus mRNA for 1A13 protein.

TESTI4010382//cytoplasmic dynein heavy chain 2 [Rattus norvegicus]

TESTI4011072//tudor domain containing 1 [Mus musculus]

TESTI4046240//sirtuin 7

UTERU2019534//Golgi apparatus protein 1 [Homo sapiens]

UTERU2028734//Mus musculus slp2-a mRNA for synaptotagmin-like protein 2-a delta 2S-III, complete cds.

So far no information suggesting the function of the remaining 1,061 clones has been provided by the homology search. The functions of these clones may be clarified when an updated database becomes available in future. Clone names are shown below.

3NB692008729, ADRGL2012038, ADRGL2012179, ASTRO2003960, ASTRO2018373, ASTRO3000172, ASTRO3000482, BLADE2001371, BLADE2001987, BLADE2003474, BLADE2005459, BLADE2007666, BLADE2008281, BRACE1000258, BRACE1000533, BRACE1000572, BRACE2003639, BRACE2011747, BRACE2014475, BRACE2014657, BRACE2015058, BRACE2015314, BRACE2024627, BRACE2027970, BRACE2028970, BRACE2029849, BRACE2031154, BRACE2031389, BRACE2032044, BRACE2032329, BRACE2032538, BRACE2032823, BRACE2033720, BRACE2035441, BRACE2036096, BRACE2036830, BRACE2036834, BRACE2037847, BRACE2038114, BRACE2039475, BRACE2039734, BRACE2040325, BRACE2041009, BRACE2041264, BRACE2043349, BRACE2044816, BRACE2044949, BRACE2045428, BRACE2045947, BRACE2045954, BRACE2047011, BRACE2047350, BRACE2047385, BRACE3000071, BRACE3000787, BRACE3000973, BRACE3001217, BRACE3001595, BRACE3002390, BRACE3003698, BRACE3004358, BRACE3004435, BRACE3004783, BRACE3004880, BRACE3005145, BRACE3005225, BRACE3005499, BRACE3006185, BRACE3006226, BRACE3007322, BRACE3007472, BRACE3007480, BRACE3007559, BRACE3007642, BRACE3007767, BRACE3008036, BRACE3008092, BRACE3008772, BRACE3009090, BRACE3009237, BRACE3009377, BRACE3009701, BRACE3009724, BRACE3010428, BRACE3012364, BRACE3012930, BRACE3013119, BRACE3013576, BRACE3013780, BRACE3014231, BRACE3014317, BRACE3015121, BRACE3015894, BRACE3018308, BRACE3019055, BRACE3020194, BRACE3022769, BRACE3023912, BRACE3024659, BRACE3025153, BRACE3026075, BRACE3027242, BRACE3027478, BRACE3030103, BRACE3032983, BRALZ2011796, BRALZ2012183, BRALZ2012848, BRALZ2014484, BRAMY2003008, BRAMY2019985, BRAMY2020058, BRAMY2020270, BRAMY2028914, BRAMY2029602, BRAMY2030098, BRAMY2030702, BRAMY2030703, BRAMY2030799, BRAMY2031377, BRAMY2031442, BRAMY2032014, BRAMY2032242, BRAMY2032317, BRAMY2034185, BRAMY2034920, BRAMY2034993, BRAMY2036387, BRAMY2036699, BRAMY2036913, BRAMY2037823, BRAMY2038100, BRAMY2038846, BRAMY2040478, BRAMY2041261, BRAMY2041378, BRAMY2042612, BRAMY2042641, BRAMY2042760, BRAMY2042918, BRAMY2044078, BRAMY2044246, BRAMY2046478, BRAMY2046742, BRAMY2047169, BRAMY2047676, BRAMY2047884, BRAMY3000206, BRAMY3001401, BRAMY3002620, BRAMY3002805, BRAMY3004672, BRAMY3004900, BRAMY3006297, BRAMY3008650, BRAMY3009811, BRAMY3010411, BRASW1000053, BRAWH2001671, BRAWH2001973, BRAWH2010084, BRAWH2012326, BRAWH2013294, BRAWH2013871, BRAWH2014645, BRAWH2014662, BRAWH2014876, BRAWH3000345, BRAWH3002819, BRAWH3003522, BRAWH3003801, BRAWH3005422, BRAWH3005981, BRAWH3006792, BRAWH3007726, BRAWH3007783, BRAWH3008697, BRAWH3008931, BRAWH3009297, BRCAN2003987, BRCAN2004355, BRCAN2005436, BRCAN2006290, BRCAN2006297, BRCAN2006450, BRCAN2007144, BRCAN2007409, BRCAN2007426, BRCAN2008528, BRCAN2011254, BRCAN2011602, BRCAN2014881, BRCAN2015371, BRCAN2015464, BRCAN2016433, BRCAN2017717, BRCAN2017905, BRCAN2018935, BRCAN2019387, BRCAN2020710, BRCAN2021028, BRCAN2024563, BRCAN2025712, BRCOC2000670, BRCOC2014033, BRCOC2016525, BRCOC2020142, BRHIP2000826, BRHIP2000920, BRHIP2001074, BRHIP2002346, BRHIP2003242, BRHIP2003917, BRHIP2004312, BRHIP2005354, BRHIP2005600, BRHIP2005719, BRHIP2005932, BRHIP2006800, BRHIP2007741, BRHIP2009340, BRHIP2009474, BRHIP2014228, BRHIP2022221, BRHIP2024146, BRHIP2026061, BRHIP2029176, BRHIP3001283, BRHIP3006683, BRHIP3008183, BRHIP3008344, BRHIP3008565, BRHIP3008997, BRHIP3009099, BRHIP3011241, BRHIP3013765, BRHIP3013897, BRHIP3016213, BRHIP3018797, BRHIP3020182, BRHIP3024533, BRHIP3024725, BRHIP3025702, BRSSN2006892, BRSSN2008797, BRSSN2011262, BRSSN2014556, BRSSN2018581, BRSTN2001067, BRSTN2002400, BRSTN2005721, BRSTN2006865, BRSTN2007284, BRSTN2008052, BRSTN2008283, BRSTN2008457, BRSTN2009899, BRSTN2010500, BRSTN2010750, BRSTN2012320, BRSTN2017084, BRSTN2017237, BRSTN2018083, BRSTN2019129, BRTHA1000311, BRTHA2000855, BRTHA2001462, BRTHA2002115, BRTHA2002442, BRTHA2003030, BRTHA2003116, BRTHA2004821, BRTHA2004978, BRTHA2005956, BRTHA2006146, BRTHA2006194, BRTHA2007422, BRTHA2008316, BRTHA2008335, BRTHA2008535, BRTHA2008955, BRTHA2009846, BRTHA2009972, BRTHA2010073, BRTHA2010608, BRTHA2010884, BRTHA2010907, BRTHA2011351, BRTHA2011500, BRTHA2011641, BRTHA2012562, BRTHA2013262, BRTHA2013707, BRTHA2014828, BRTHA2015478, BRTHA2016543, BRTHA2017353, BRTHA2018165, BRTHA2018591, BRTHA2018707, BRTHA2019014, BRTHA2019022, BRTHA2019048, BRTHA3000273, BRTHA3000297, BRTHA3002401, BRTHA3003490, BRTHA3004475, BRTHA3005046, BRTHA3006856, BRTHA3007113, BRTHA3007319, BRTHA3007769, BRTHA3008143, BRTHA3010366, BRTHA3015910, BRTHA3018514, BRTHA3018617, BRTHA3019105, CERVX1000042, COLON1000030, COLON2003043, COLON2006282, COLON2009499, CORDB1000140, CORDB2000061, CTONG1000087, CTONG1000088, CTONG1000288, CTONG1000540, CTONG2001877, CTONG2019248, CTONG2019704, CTONG2019833, CTONG2020806, CTONG2021132, CTONG2022153, CTONG2024206, CTONG2025900, CTONG3000657, CTONG3000686, CTONG3000896, CTONG3001420, CTONG3002674, CTONG3003652, CTONG3003654, CTONG3003905, CTONG3003972, CTONG3004712, CTONG3005325, CTONG3005713, CTONG3005813, CTONG3006067, CTONG3006650, CTONG3007444, CTONG3007528, CTONG3007586, CTONG3007870, DFNES2011239, DFNES2011499, ERLTF2000324, FCBBF3001977, FCBBF3004847, FCBBF3006171, FCBBF3007242, FCBBF3008944, FCBBF3012288, FCBBF3013846, FCBBF3023443, FCBBF3025730, FCBBF3027717, FEBRA2000253, FEBRA2006396, FEBRA2007793, FEBRA2008287, FEBRA2008360, FEBRA2010719, FEBRA2014213, FEBRA2015588, FEBRA2020484, FEBRA2020582, FEBRA2020886, FEBRA2021339, FEBRA2021571, FEBRA2021908, FEBRA2021966, FEBRA2024136, FEBRA2024150, FEBRA2024343, FEBRA2027297, FEBRA2027352, FEBRA2028366, FEBRA2028477, FEBRA2028618, HCASM2002918, HCASM2003415, HCHON2000244, HCHON2000418, HCHON2006714, HCHON2007881, HEART1000088, HEART2001756, HEART2007031, HEART2010391, HHDPC2001337, HLUNG1000017, HLUNG2001996, HLUNG2002958, HLUNG2003003, HLUNG2012049, HLUNG2012287, HLUNG2012727, HLUNG2013304, HLUNG2013622, HLUNG2013851, HLUNG2014262, HLUNG2014449, HLUNG2017546, HLUNG2017806, HLUNG2019058, HSYRA2004858, HSYRA2005456, HSYRA2007667, HSYRA2008376, HSYRA2009075, IMR322002035, IMR322003675, IMR322007225, IMR322016146, IMR322018117, KIDNE2000665, KIDNE2000722, KIDNE2002991, KIDNE2003837, KIDNE2005543, KIDNE2011314, LYMPB1000141, MESAN2006563, MESAN2014295, MESAN2015515, MESAN2018576, MESTC2000153, NB9N41000340, NCRRP1000129, NESOP2000744, NESOP2001656, NESOP2001752, NESOP2002738, NHNPC2000877, NHNPC2001816, NHNPC2002565, NHNPC2002749, NOVAR2000710, NOVAR2000962, NOVAR2001783, NT2NE2005890, NT2NE2008060, NT2RI2003993, NT2RI2011683, NT2RI2012659, NT2RI2013357, NT2RI2014247, NT2RI2014551, NT2RI2014733, NT2RI2016128, NT2RI2018311, NT2RI2018883, NT2RI2019751, NT2RI2023303, NT2RI2027081, NT2RI2027396, NT2RI3000622, NT2RI3002303, NT2RI3002842, NT2RI3002892, NT2RI3003031, NT2RI3003162, NT2RI3004381, NT2RI3004510, NT2RI3005202, NT2RI3005724, NT2RI3006132, NT2RI3006376, NT2RI3006796, NT2RI3007158, NT2RI3007291, NT2RI3008055, NT2RI3008162, NT2RP7004123, NT2RP7005846, NT2RP7009867, NT2RP7010128, NT2RP7011570, NT2RP7015512, NT2RP7017365, NT2RP7017474, NT2RP7017546, NT2RP8000137, NTONG2003852, NTONG2005969, NTONG2006354, NTONG2007249, NTONG2008088, OCBBF1000254, OCBBF2001794, OCBBF2003819, OCBBF2004883, OCBBF2005428, OCBBF2007478, OCBBF2008770, OCBBF2009788, OCBBF2010416, OCBBF2019327, OCBBF2020343, OCBBF2020453, OCBBF2020639, OCBBF2020741, OCBBF2021286, OCBBF2022574, OCBBF2023162, OCBBF2023643, OCBBF2024781, OCBBF2024850, OCBBF2025028, OCBBF2025730, OCBBF2026645, OCBBF2027423, OCBBF2027478, OCBBF2028935, OCBBF2029901, OCBBF2030708, OCBBF2031366, OCBBF2032599, OCBBF2032611, OCBBF2032671, OCBBF2035110, OCBBF2035214, OCBBF2035564, OCBBF2035885, OCBBF2035916, OCBBF2036476, OCBBF2037398, OCBBF2037638, OCBBF3000296, OCBBF3002553, OCBBF3002600, OCBBF3006802, OCBBF3007516, OCBBF3008230, OCBBF3009279, PEBLM2001488, PEBLM2002749, PEBLM2004497, PEBLM2004666, PEBLM2005697, PEBLM2007112, PEBLM2007140, PERIC1000147, PERIC2000914, PERIC2001228, PERIC2002766, PERIC2003090, PERIC2003699, PERIC2003834, PERIC2004259, PERIC2004379, PERIC2004429, PERIC2005370, PLACE5000001, PLACE6001185, PLACE6009006, PLACE7001936, PLACE7008623, PROST2015243, PROST2017413, PROST2017700, PROST2018030, PROST2019296, PROST2019781, PUAEN2002616, PUAEN2005930, PUAEN2007785, SKMUS2003074, SKMUS2004047, SKNMC2004457, SKNMC2004643, SKNMC2005772, SKNMC2007961, SKNMC2009450, SKNSH2000482, SKNSH2009991, SKNSH2010015, SMINT2001818, SMINT2006641, SMINT2007391, SMINT2010897, SMINT2011311, SPLEN2002707, SPLEN2014946, SPLEN2016554, SPLEN2016863, SPLEN2017104, SPLEN2024127, SPLEN2027268, SPLEN2028844, SPLEN2028914, SPLEN2029051, SPLEN2029176, SPLEN2029727, SPLEN2029912, SPLEN2032321, SPLEN2033153, SPLEN2033539, SPLEN2033921, SPLEN2034081, SPLEN2034678, SPLEN2036103, SPLEN2036712, SPLEN2037580, SPLEN2037630, SPLEN2038055, SPLEN2039697, SPLEN2039936, SPLEN2041310, SPLEN2041645, SPLEN2041720, SPLEN2041977, SPLEN2042303, STOMA1000189, STOMA2003444, STOMA2004925, SYNOV1000374, SYNOV2005448, SYNOV2006430, SYNOV2017055, SYNOV2018921, SYNOV4000472, SYNOV4001326, SYNOV4001395, SYNOV4002346, SYNOV4002392, SYNOV4004823, SYNOV4007012, SYNOV4007215, T1ESE2000116, TBAES2001171, TBAES2001220, TBAES2001492, TBAES2001751, TBAES2002197, TBAES2003550, TBAES2005157, TBAES2005543, TBAES2006568, TBAES2007964, TCOLN2002278, TESOP1000127, TESOP2001818, TESOP2001849, TESOP2002451, TESOP2002489, TESOP2002950, TESOP2003273, TESOP2003753, TESOP2006060, TESOP2006068, TESOP2006670, TESOP2006746, TESOP2007688, TESOP2009555, TESTI1000330, TESTI1000390, TESTI1000491, TESTI2000443, TESTI2004700, TESTI2005376, TESTI2005986, TESTI2006041, TESTI2006643, TESTI2009474, TESTI2009511, TESTI2009812, TESTI2010400, TESTI2013381, TESTI2014843, TESTI2017727, TESTI2018838, TESTI2019042, TESTI2019648, TESTI2023254, TESTI2023599, TESTI2031529, TESTI2034749, TESTI2035107, TESTI2036513, TESTI2037643, TESTI2044833, TESTI2045920, TESTI2045983, TESTI2046347, TESTI2048465, TESTI2048603, TESTI2049206, TESTI2049246, TESTI2049277, TESTI2049422, TESTI2049452, TESTI2049469, TESTI2049576, TESTI2050681, TESTI2051279, TESTI2051488, TESTI2051543, TESTI2051806, TESTI2052211, TESTI2052698, TESTI2053242, TESTI2053526, TESTI4000068, TESTI4000250, TESTI4000462, TESTI4000530, TESTI4000970, TESTI4001201, TESTI4001206, TESTI4002290, TESTI4002647, TESTI4002703, TESTI4002878, TESTI4004200, TESTI4005628, TESTI4005857, TESTI4006112, TESTI4006219, TESTI4006326, TESTI4006412, TESTI4007203, TESTI4007373, TESTI4007404, TESTI4007489, TESTI4007775, TESTI4007799, TESTI4008007, TESTI4008219, TESTI4008573, TESTI4008816, TESTI4008935, TESTI4008993, TESTI4009022, TESTI4009034, TESTI4009123, TESTI4009215, TESTI4009283, TESTI4009406, TESTI4009638, TESTI4010211, TESTI4010377, TESTI4010713, TESTI4010789, TESTI4010817, TESTI4010928, TESTI4011118, TESTI4011161, TESTI4011246, TESTI4011505, TESTI4012086, TESTI4012329, TESTI4012556, TESTI4012702, TESTI4013675, TESTI4013685, TESTI4013735, TESTI4014159, TESTI4014306, TESTI4014445, TESTI4014694, TESTI4015263, TESTI4015293, TESTI4015471, TESTI4015600, TESTI4015646, TESTI4015681, TESTI4015688, TESTI4016551, TESTI4016812, TESTI4016882, TESTI4017001, TESTI4017137, TESTI4017254, TESTI4017848, TESTI4017961, TESTI4018382, TESTI4018555, TESTI4018806, TESTI4019299, TESTI4019417, TESTI4020806, TESTI4020920, TESTI4021294, TESTI4021456, TESTI4021491, TESTI4022936, TESTI4023555, TESTI4023942, TESTI4024344, TESTI4024907, TESTI4025731, TESTI4026079, TESTI4026192, TESTI4026295, TESTI4026700, TESTI4026762, TESTI4027516, TESTI4028612, TESTI4028809, TESTI4028983, TESTI4029370, TESTI4029671, TESTI4030603, TESTI4030669, TESTI4032895, TESTI4033433, TESTI4033690, TESTI4034212, TESTI4034432, TESTI4035602, TESTI4035637, TESTI4036042, TESTI4037066, TESTI4037188, TESTI4038156, TESTI4038223, TESTI4038258, TESTI4038339, TESTI4038492, TESTI4038818, TESTI4039038, TESTI4039086, TESTI4040800, TESTI4040939, TESTI4040956, TESTI4041099, TESTI4041143, TESTI4041519, TESTI4041624, TESTI4041903, TESTI4041954, TESTI4042098, TESTI4042444, TESTI4043129, TESTI4043203, TESTI4043551, TESTI4043947, TESTI4044035, TESTI4044084, TESTI4044123, TESTI4044234, TESTI4044296, TESTI4044682, TESTI4045312, TESTI4046253, TESTI4046282, TESTI4046884, TESTI4047069, THYMU1000600, THYMU2000932, THYMU2001090, THYMU2003397, THYMU2004693, THYMU2005190, THYMU2007179, THYMU2008282, THYMU2009134, THYMU2016204, THYMU2016523, THYMU2019587, THYMU2023967, THYMU2025707, THYMU2028978, THYMU2029688, THYMU2030226, THYMU2030264, THYMU2030637, THYMU2030796, THYMU2031218, THYMU2031341, THYMU2031368, THYMU2031579, THYMU2031847, THYMU2031890, THYMU2032080, THYMU2032358, THYMU2032696, THYMU2033070, THYMU2033787, THYMU2034647, THYMU2035101, THYMU2035388, THYMU2035400, THYMU2036058, THYMU2036252, THYMU2036265, THYMU2036653, THYMU2037081, THYMU2037208, THYMU2037965, THYMU2038189, THYMU2038615, THYMU2038636, THYMU2038739, THYMU2039350, THYMU2039411, THYMU2039989, THYMU2040140, THYMU2040824, THYMU2041007, THYMU2041252, THYMU3000133, THYMU3000655, THYMU3000826, THYMU3001472, THYMU3002452, THYMU3002661, THYMU3003763, THYMU3006132, THYMU3006371, THYMU3006485, THYMU3006963, THYMU3008171, THYMU3009255, TKIDN2003044, TKIDN2004386, TKIDN2005934, TKIDN2005947, TKIDN2007667, TKIDN2009641, TKIDN2010934, TKIDN2012824, TKIDN2013287, TKIDN2014757, TKIDN2015263, TKIDN2015788, TKIDN2016309, TLIVE2002338, TLIVE2003197, TLIVE2003970, TLIVE2004601, TLIVE2005180, TLIVE2006236, TLIVE2006529, TLIVE2007132, TLIVE2007528, TLIVE2007816, TLIVE2008083, TLIVE2009541, TOVAR2000649, TOVAR2001730, TOVAR2002549, TRACH2001443, TRACH2003070, TRACH2004170, TRACH2005066, TRACH2005811, TRACH2006049, TRACH2019248, TRACH2019473, TRACH2021398, TRACH2022042, TRACH2023306, TRACH2025344, TRACH2025749, TRACH2025932, TRACH3000342, TRACH3000586, TRACH3002192, TRACH3002866, TRACH3002871, TRACH3003379, TRACH3004068, TRACH3004537, TRACH3004840, TRACH3005479, TRACH3006038, TRACH3006228, TRACH3006470, TRACH3006889, TRACH3008093, TRACH3008535, TRACH3034762, TRACH3035235, TRACH3035482, TRACH3036207, TRACH3036309, TRACH3036456, TSTOM1000135, TSTOM2002672, TUTER1000122, TUTER2001387, UTERU1000024, UTERU1000148, UTERU1000249, UTERU2000649, UTERU2001409, UTERU2002410, UTERU2002841, UTERU2005004, UTERU2007520, UTERU2014678, UTERU2019681, UTERU2020491, UTERU2021163, UTERU2021380, UTERU2022981, UTERU2023039, UTERU2023175, UTERU2023651, UTERU2023712, UTERU2024002, UTERU2024656, UTERU2030213, UTERU2030280, UTERU2031084, UTERU2031521, UTERU2031703, UTERU2033375, UTERU2033382, UTERU2035114, UTERU2035331, UTERU2035503, UTERU2037361, UTERU2037577, UTERU2038251, UTERU3000226, UTERU3001542, UTERU3001766, UTERU3002218, UTERU3002667, UTERU3002731, UTERU3002768, UTERU3002993, UTERU3003465, UTERU3003523, UTERU3004523, UTERU3004616, UTERU3004709, UTERU3005049, UTERU3005205, UTERU3005230, UTERU3005460, UTERU3005970, UTERU3006008, UTERU3007134, UTERU3007913, UTERU3008660, UTERU3009259, UTERU3009517, UTERU3015086, UTERU3018081, UTERU3018154, UTERU3018616, UTERU3018711

Likewise, so far no information suggesting the function of the 72 clones shown below has been provided by the homology search. The functions of these clones may be clarified when an updated database becomes available in future. Clone names are shown below.

3NB692004724, ADRGL2000056, BLADE2000579, BRACE2037299, BRACE2043105, BRACE3003026, BRACE3003053, BRACE3005107, BRACE3015829, BRAMY2041434, BRAWH2009393, BRAWH2010552, BRCAN2014229, BRHIP2002722, BRHIP3000017, BRTHA2002133, BRTHA2002702, BRTHA2010033, BRTHA2013426, BRTHA3000296, CTONG2004000, CTONG2015596, CTONG2020374, CTONG2024031, CTONG3002552, CTONG3003598, CTONG3009287, HCASM2003357, HCHON2000743, HLUNG2016862, IMR322001879, IMR322007078, NT2RI3002557, NT2RI3005928, NT2RI3007167, NT2RP8000521, OCBBF2006987, OCBBF3003761, OCBBF3004972, PLACE7000333, PUAEN2006335, SKMUS2003194, SPLEN2033490, STOMA2004893, SYNOV2006620, SYNOV4005739, TESTI1000266, TESTI2008901, TESTI2035981, TESTI2037830, TESTI4001984, TESTI4008058, TESTI4011829, TESTI4013602, TESTI4015012, TESTI4025865, TESTI4028958, TESTI4029348, TESTI4029528, TESTI4029690, TESTI4036767, TESTI4038721, THYMU2032976, THYMU3000360, THYMU3001428, TRACH1000212, UTERU2004299, UTERU2035978, UTERU3000402, UTERU3014791, UTERU3015412, UTERU3017176

### EXAMPLE 7

### Functional Categorization Based on a Functional Domain Search for Deduced Amino Acid Sequences

Domains and motifs are minimal functional structures of polypeptides. The structure of a polypeptide is constituted by a collection of such minimal structures, and thus the overall function of a polypeptide is ensured by the resulting structure. Thus, the overall function of a polypeptide can be predicted relatively accurately using data obtained by analysis of domain and motif structures. Furthermore, classifying these results into functional categories in a database allows clones comprising specific functions to be easily selected. Thus, such databases are highly useful in the functional analysis of each clone.

Pfam was used to undertake a domain search for the amino acid sequences deduced from the full-length nucleotide sequences (see Example 5). Based on these results, the proteins encoded by clones 664 and 250 were categorized and their functions predicted. This was performed by referring to domain and motif names, accession numbers for hit data, and detailed descriptions in Pfam (http://www.sanger.ac.uk/Software/Pfam/index.shtml) as well as functional categorizations in PROSITE (http://www.expasy.ch/cgi-bin/prosite-list.pl).

A clone predicted to belong to the category of secretory and/or membrane protein means a clone having domains and motifs, for example, seven-transmembrane receptor, pancreatic hormone peptides, ion transport protein, or fibroblast growth factor, which suggest receptor, ion channel, hormone, or growth factor.

A clone predicted to belong to the category of glycoprotein-related protein means a clone having domains and motifs, for example, immunoglobulin domain or glycosyl transferases group 1, which suggest involvement in glycobiology, such as glycoprotein or glycosyltransferase.

A clone predicted to belong to the category of signal transduction-related protein means a clone having domains and motifs, for example, eukaryotic protein kinase domain, protein phosphatase 2C, or Ras family, which suggest protein kinase, dephosphoenzyme, SH2 domain, or small G protein.

A clone predicted to belong to the category of transcription-related protein means a clone having domains and motifs, for example, bZIP transcription factor, Zinc finger, or C2H2 type, which suggest transcription factor or transcription-controlling protein.

A clone predicted to belong to the category of disease-related protein means a clone having domains and motifs, for example, Wilm's tumor protein or von Hippel-Lindau disease tumor suppressor protein, which suggest proteins with disease-specific expression or that promote or suppress expression, depending on the disease.

A clone predicted to belong to the category of enzyme and/or metabolism-related protein means a clone having domains and motifs, for example, aldehyde dehydrogenase family, chitin synthase, or glucose-6-phosphate dehydrogenase, which suggest transferase, synthase, or hydrolase.

A clone predicted to belong to the category of cell division- and/or cell proliferation-related protein means a clone having domains and motifs, for example, cyclin or cell division protein, which suggest cyclin or cell proliferation-controlling protein.

A clone predicted to belong to the category of cytoskeleton-related protein means a clone having domains and motifs, for example, actin, fibronectin type I domain, or kinesin motor domain, which suggest actin, kinesin, or fibronectin.

A clone predicted to belong to the category of nuclear protein and/or RNA synthesis-related protein means a clone having domains and motifs, for example, hepatitis C virus RNA dependent RNA polymerase or DEAD/DEAH box helicase, which suggest splicing factor, RNA synthase, or helicase.

A clone predicted to belong to the category of protein synthesis and/or transport-related protein means a clone having domains and motifs, for example, translation initiation factor SUI1, ubiquitin family, or ribosomal protein L16, which suggest translation-related protein, ubiquitin-related protein, or ribosomal protein.

A clone predicted to belong to the category of cellular defense-related protein means a clone having domains and motifs, for example, HSP90 protein or DNA mismatch repair protein, which suggest chaperonin or DNA repair protein.

A clone predicted to belong to the category of development- and/or differentiation-related proteins means a clone having domains and motifs, for example, floricaula/leafy protein, which suggest organogenesis-related protein.

A clone predicted to belong to the category of DNA- and/or RNA-binding protein means a clone having domains and motifs, for example, transcription factor WhiB, B-box zinc finger, or tRNA synthetases class I (C), which suggest DNA/RNA-relating enzyme group including transcription factor and DNA ligase or Zinc-finger related protein.

A clone predicted to belong to the category of ATP- and/or GTP-binding protein means a clone having domains and motifs, for example, E1-E2 ATPase or Ras family, which suggest ATP/GTP-related enzyme group including ATPase or G protein.

During this functional categorization, if a clone met every criterion of multiple categories as described above, it was grouped into multiple categories. However, the function of a polypeptide is not limited to these functional categories.

The clones predicted to belong to the category of secretory protein and/or membrane protein are the following 64 clones.

ASTRO2014923, ASTRO3000301, BRACE2005457, BRACE2014306, BRACE3001391, BRACE3014005, BRALZ2016085, BRAMY2040592, BRAWH2014662, BRHIP2004814, BRHIP3024118, BRTHA3002427, BRTHA3017848, BRTHA3018656, CTONG2009423, CTONG2013178, D3OST2002648, FEBRA2007708, FEBRA2008311, HCHON2001084, HCHON2001712, HCHON2004531, HCHON2005921, HSYRA2009102, KIDNE1000064, KIDNE2000832, NT2RI3006376, OCBBF2031167, OCBBF2035110, OCBBF2038317, PEBLM2002594, PERIC1000147, PERIC2009086, PROST1000184, SPLEN2012624, SPLEN2031547, SPLEN2033098, SPLEN2036326, TESTI1000257, TESTI1000390, TESTI2000644, TESTI2002036, TESTI2002928, TESTI2006648, TESTI2024567, TESTI2034520, TESTI4000014, TESTI4000724, TESTI4007163, TESTI4009881, TESTI4028880, THYMU2009425, THYMU2011548, THYMU2033079, THYMU2041015, TLIVE2000023, TLIVE2003381, TLIVE2007132, TRACH2006387, TRACH2007059, TRACH3004786, UTERU3000645, UTERU3004616, UTERU3006308

The following 23 clones are also predicted to belong to the category of secretory protein and/or membrane protein.

BRACE2029396, BRACE3005107, BRACE3010076, BRAMY2019111, BRAMY3004800, BRHIP3000017, FCBBF1000509, HCHON2000508, HEART2009680, IMR322013396, NT2RI2009583, NT2RI3000174, NT2RP8000521, OCBBF2030116, TESTI2029252, TESTI4013894, TESTI4032112, TESTI4041086, THYMU2035710, TKIDN2012771, TRACH3000420, UTERU2004299, TESTI4038779

The clones predicted to belong to the category of glycoprotein-related protein are the following 77 clones.

ADIPS2000088, BRACE2043142, BRACE2046295, BRACE3014005, BRAMY2005052, BRAMY4000277, BRAWH2007658, BRCAN2006063, BRSTN2004863, BRTHA3017589, BRTHA3017848, COLON2000568, COLON2004478, CTONG2000042, CTONG2013178, CTONG2024206, CTONG2024749, CTONG2025496, CTONG3001370, CTONG3003737, D3OST2002182, FEBRA2007708, HCHON2001084, HCHON2002676, HCHON2004531, HEART2001680, HLUNG2014262, LYMPB2000083, NESOP2001433, NOVAR2001108, NT2RI3006171, NT2RI3006340, NT2RI3007978, NT2RP7014005, OCBBF2010140, OCBBF2037598, PLACE5000171, PLACE6012574, PLACE7006051, PUAEN2009174, SMINT2002743, SMINT2010076, SMINT2011888, SMINT2015787, SPLEN2001599, SPLEN2015267, SPLEN2021701, SPLEN2037722, STOMA2004294, SYNOV3000231, SYNOV3000302, SYNOV4007521, SYNOV4007671, TBAES2003550, TESOP2005485, TESTI2005610, TESTI4006326, TESTI4021294, THYMU2005303, THYMU2019210, THYMU2023711, THYMU2027695, TRACH2007059, TRACH2022425, TRACH2022553, TRACH2022649, TRACH3002168, TRACH3005479, TRACH3005549, TRACH3006470, TRACH3035526, TRACH3036609, TSTOM2000442, UTERU2026090, UTERU3004616, UTERU3004992, UTERU3006308

The following eight clones are also predicted to belong to the category of glycoprotein-related protein.

BRAWH2006395, BRHIP3000017, NT2RI3007443, OCBBF3002654, TESTI2039060, TESTI4013894, TESTI4031745, TLIVE2001684

The clones predicted to belong to the category of signal transduction-related protein are the following 116 clones.

BLADE2007958, BNGH42007788, BRACE1000258, BRACE2008594, BRACE2041009, BRACE3001391, BRACE3006872, BRACE3011421, BRACE3024073, BRACE3027326, BRALZ2014484, BRAMY2001473, BRAMY2036567, BRAMY2042760, BRAMY2047751, BRAMY3001794, BRAMY3002803, BRAMY3005091, BRAMY3008466, BRAMY4000095, BRAWH3001326, BRAWH3002821, BRAWH3005912, BRCAN2002856, BRCAN2009432, BRCAN2016619, BRCAN2024451, BRCAN2028355, BRHIP2000819, BRHIP2005932, BRHIP3008405, BRHIP3025161, BRSSN2000684, BRSSN2004719, BRSTN2008418, BRSTN2013741, BRTHA3009037, BRTHA3013884, COLON2001721, CTONG2006798, CTONG3000084, CTONG3000657, CTONG3002127, D3OST3000169, DFNES2001108, DFNES2011499, FCBBF3007540, HCASM2001301, HCHON2000028, HCHON2006250, HHDPC1000118, HLUNG2001996, HLUNG2002465, KIDNE2001847, MESAN2006563, NHNPC2001816, NT2NE2003252, NT2RI2005166, NT2RI3000622, NT2RI3006673, NT2RP7005118, NT2RP7005529, NT2RP7009147, NT2RP7013795, NT2RP8000483, NTONG2003852, OCBBF2004826, OCBBF2004883, OCBBF2007028, OCBBF2008770, OCBBF2022351, OCBBF2037340, OCBBF2037547, PEBLM2004666, PLACE7008431, PROST2016462, PROST2018511, PUAEN2002616, PUAEN2005930, PUAEN2006328, PUAEN2009852, SYNOV2021320, TESOP2000801, TESOP2001166, TESTI2006648, TESTI2026505, TESTI2050137, TESTI2052693, TESTI4000079, TESTI4010713, TESTI4010831, TESTI4011956, TESTI4016882, TESTI4019843, TESTI4028059, THYMU2032014, THYMU2037226, THYMU2038615, THYMU3001234, THYMU3006172, THYMU3008436, TLIVE2009541, TRACH2009310, TRACH2021398, TRACH2023299, TRACH2025535, TRACH3009455, TRACH3034731, TSTOM2000553, UTERU1000337, UTERU2005621, UTERU2025025, UTERU2036089, UTERU2038251, UTERU3003523, UTERU3007419

The following 38 clones are also predicted to belong to the category of signal transduction-related protein.

BLADE2000579, BRACE3001058, BRACE3003053, BRACE3009127, BRAMY2040159, BRAMY3004800, BRAWH3009017, BRCAN2014229, BRHIP2026877, BRTHA2013610, CTONG3004550, FEBRA2001990, FEBRA2008692, HCHON2000508, MESAN2001770, NT2RI2005772, NT2RI3007443, NTONG2008093, OCBBF2005433, OCBBF2024284, OCBBF2034637, OCBBF3002654, TESOP2000390, TESTI2025924, TESTI2049956, TESTI4000319, TESTI4005317, TESTI4021482, TESTI4025268, TESTI4031745, THYMU2004139, THYMU2031249, TRACH2024408, UTERU2008040, UTERU2028734, UTERU3000402, UTERU3000738, UTERU3015412

The clones predicted to belong to the category of transcription-related protein are the following 27 clones.

BRACE2006319, BRACE3013576, BRAMY2030109, BRAWH3005912, BRHIP3025161, CORDB1000140, CTONG1000467, HEART2001756, IMR322000127, IMR322000917, KIDNE1000064, NOVAR2000136, NT2NE2006531, NT2RI3007158, NT2RP7000466, OCBBF2036743, OCBBF3009279, PLACE6019385, TESTI2026505, TESTI2044796, TESTI2050987, TESTI4017001, TESTI4019140, TESTI4034912, THYMU2035735, TRACH2025749, TRACH3004840

The following 88 clones are also predicted to belong to the category of transcription-related protein.

BRACE2003609, BRACE3001058, BRACE3001113, BRACE3003026, BRAMY2035070, BRAMY2035449, BRAMY2035718, BRAMY2039341, BRAMY2045471, BRAWH3007441, BRHIP2017553, BRSTN2013354, CERVX2002013, CTONG1000113, CTONG2003348, CTONG2020374, CTONG2020378, CTONG2020411, CTONG2024031, CTONG2028758, CTONG3001501, CTONG3004726, DFNES2011192, FCBBF3027854, FEBRA2014122, FEBRA2027609, HCASM2003018, HCASM2003099, HCHON2000508, HCHON2000743, HCHON2004858, HSYRA2005628, MESAN2014412, MESAN2015501, NT2RI2008952, NT2RI2018448, NT2RI3000174, NT2RI3001132, NT2RI3002557, NT2RI3007167, NT2RI3007443, OCBBF2008144, OCBBF2009583, OCBBF2011669, OCBBF2019684, OCBBF2020048, OCBBF2024284, OCBBF2032274, OCBBF3000167, OCBBF3003761, SPLEN2016135, SPLEN2016781, SPLEN2036702, SYNOV2021953, SYNOV4002744, TESOP2001796, TESOP2005199, TESOP2006398, TESTI2008901, TESTI2034251, TESTI2037830, TESTI4000183, TESTI4000214, TESTI4006473, TESTI4008058, TESTI4008302, TESTI4013365, TESTI4014801, TESTI4015442, TESTI4017714, TESTI4025494, TESTI4025547, TESTI4028938, TESTI4029348, TESTI4031745, TESTI4032090, THYMU2006001, THYMU2028739, THYMU2031139, THYMU3001428, TRACH2007483, TRACH3000134, TRACH3003832, TRACH3007866, UTERU3001053, UTERU3014791, UTERU3017176, TESTI4038779

The clones predicted to belong to the category of enzyme and/or metabolism-related protein are the following 176 clones.

3NB692002806, ASTRO1000009, BLADE2005036, BLADE2008539, BRACE2005457, BRACE2008594, BRACE2014475, BRACE2018762, BRACE2035381, BRACE2043142, BRACE2047011, BRACE3004058, BRACE3007625, BRACE3009708, BRACE3011421, BRACE3015262, BRACE3024073, BRACE3025630, BRACE3027478, BRAMY2047746, BRAMY2047751, BRAMY3002803, BRAMY3004919, BRAMY3005091, BRAMY4000095, BRAWH2010000, BRAWH2014414, BRAWH2014662, BRAWH2016702, BRAWH3002821, BRAWH3003727, BRCAN2021028, BRCAN2024451, BRCAN2028355, BRCOC2003213, BRHIP2004359, BRHIP2026288, BRHIP3008183, BRHIP3025161, BRHIP3027137, BRSSN2000684, BRSTN2000872, BRSTN2004863, BRSTN2004987, BRTHA2012980, BRTHA3002401, BRTHA3008778, BRTHA3009037, BRTHA3009090, BRTHA3015815, BRTHA3016917, BRTHA3017848, BRTHA3018656, COLON2001721, CTONG2004062, CTONG2006798, CTONG2013178, CTONG2028124, CTONG3002127, CTONG3005325, CTONG3005648, D3OST2002182, FCBBF3004502, FCBBF3013307, FEBRA2007708, FEBRA2008468, FEBRA2026984, HCASM2001301, HCASM2002918, HCHON2002676, HCHON2004007, HCHON2004531, HEART2006131, HHDPC1000118, HLUNG1000017, KIDNE2000832, KIDNE2006580, MESAN2012054, NOVAR2000136, NT2NE2003252, NT2NE2006909, NT2RI2004618, NT2RI3004510, NT2RI3006673, NT2RI3007978, NT2RI3008652, NT2RP7010599, NT2RP7014005, NT2RP7017474, NTONG2000413, OCBBF2004826, OCBBF2006058, OCBBF2019823, OCBBF2025527, OCBBF2031167, OCBBF2037340, OCBBF2037547, OCBBF2037638, PERIC2009086, PLACE7002641, PLACE7008431, PROST2017367, PUAEN2007044, PUAEN2009795, PUAEN2009852, SPLEN2010912, SPLEN2015679, SPLEN2030335, SYNOV4002392, SYNOV4002883, TBAES2003550, TESOP2000801, TESOP2004114, TESOP2009121, TESTI1000257, TESTI1000545, TESTI2002618, TESTI2006648, TESTI2040018, TESTI2049469, TESTI2053621, TESTI4000288, TESTI4000349, TESTI4001148, TESTI4001527, TESTI4001561, TESTI4002552, TESTI4006819, TESTI4007382, TESTI4007810, TESTI4008429, TESTI4010713, TESTI4010851, TESTI4012448, TESTI4012679, TESTI4013369, TESTI4016925, TESTI4018835, TESTI4020920, TESTI4021478, TESTI4022716, TESTI4026510, TESTI4028059, TESTI4029836, TESTI4032895, TESTI4034432, TESTI4036909, THYMU2006420, THYMU3000133, THYMU3001379, THYMU3004835, THYMU3006172, THYMU3008436, TLIVE2002336, TRACH2006387, TRACH2009310, TRACH2019473, TRACH2022425, TRACH2023299, TRACH3005479, TRACH3006470, TRACH3007479, TRACH3008093, TRACH3008629, TRACH3036193, TSTOM2000553, UTERU2005621, UTERU2017762, UTERU2025025, UTERU2033375, UTERU3000828, UTERU3001240, UTERU3001585, UTERU3003116, UTERU3005460, UTERU3005907

The following 89 clones are also predicted to belong to the category of enzyme and/or metabolism-related protein.

BLADE2000579, BRACE2039823, BRACE3003053, BRAMY2038516, BRAMY2040159, BRAWH1000369, BRCAN2003070, BRCAN2014229, BRCOC2019841, BRHIP2005724, BRHIP2008389, BRHIP2026877, BRHIP3000240, BRHIP3026052, BRTHA2002133, BRTHA2002702, BRTHA2007060, BRTHA2010033, BRTHA2013426, BRTHA2013610, BRTHA2017364, BRTHA2018011, BRTHA3000296, CTONG2004000, CTONG2016942, CTONG2020374, CTONG2024031, CTONG3002552, CTONG3003598, CTONG3004550, FCBBF1000509, FEBRA2008692, HCASM2002754, HCASM2003099, HCASM2003357, HLUNG2015418, HLUNG2015548, IMR322013731, MESAN2005303, NT2RI2005772, NT2RI2008952, NT2RI3000174, NT2RI3007443, NT2RP7008435, NTONG2008093, OCBBF2006987, OCBBF2034637, OCBBF3002654, PLACE7000333, PLACE7000502, PROST2000452, SPLEN2039311, STOMA2003158, SYNOV2013637, TESOP2000390, TESTI2015626, TESTI2025924, TESTI2026647, TESTI2035981, TESTI2036288, TESTI2039060, TESTI2049956, TESTI4000155, TESTI4001984, TESTI4006473, TESTI4010382, TESTI4011072, TESTI4014801, TESTI4017714, TESTI4021482, TESTI4025547, TESTI4025865, TESTI4026207, TESTI4028958, TESTI4029690, TESTI4031745, TESTI4032090, THYMU2004139, THYMU2031139, THYMU2031249, THYMU2040925, TKIDN2012771, TLIVE2002046, TLIVE2007607, TRACH3000420, TRACH3007866, UTERU2019534, UTERU2028734, UTERU3000738

The clones predicted to belong to the category of cell division and/or cell proliferation-related protein are the following ten clones.

BRAWH2001940, CTONG3001123, HCHON2001217, PROST2008993, TBAES2001171, TESTI4021294, TESTI4035498, UTERU1000024, UTERU3002993, UTERU3003523

The following three clones are also predicted to belong to the category of cell division and/or cell proliferation-related protein.

BRACE2029396, BRAWH2010552, TESTI4013365

The clones predicted to belong to the category of cytoskeleton-related protein are the following 36 clones.

BRACE2026836, BRACE2045300, BRAWH3000314, BRSTN2004863, BRTHA2004978, BRTHA3003449, BRTHA3005046, COLON2002520, CORDB2000541, CTONG3002674, FCBBF3012288, HCHON2001577, HLUNG2017350, HSYRA2005456, HSYRA2009075, NT2RI3006340, NT2RI3006673, NT2RI3007291, OCBBF2037598, PLACE5000282, TESTI2003347, TESTI2034767, TESTI4000288, TESTI4007778, TESTI4009160, TESTI4018886, TESTI4030603, TESTI4034632, TESTI4035063, THYMU1000496, THYMU2008725, TRACH2005811, TRACH2007059, UTERU2007724, UTERU2035745, UTERU3004616

The following four clones are also predicted to belong to the category of cytoskeleton-related protein.

NT2RI2005772, OCBBF2006987, SPLEN2030847, TESTI4026207

The clones predicted to belong to the category of nuclear protein and/or RNA synthesis-related protein are the following 20 clones.

BRACE3024073, BRAWH2001940, BRCOC2003213, BRSTN2004987, BRTHA3016917, CTONG3009028, FCBBF3013307, FEBRA2026984, SPLEN2010912, TBAES2001171, TESTI2040018, TESTI4019566, TESTI4022716, TESTI4026510, TESTI4036909, THYMU3000133, TRACH2023299, TRACH3036193, UTERU1000024, UTERU3002993

The following eleven clones are also predicted to belong to the category of nuclear protein and/or RNA synthesis-related protein.

BRACE3003053, BRCAN2002473, BRTHA2017364, NT2RI2008952, NT2RI3000174, TESTI2026647, TESTI2035981, TESTI4000155, TESTI4006473, TESTI4010382, TESTI4025547

The clones predicted to belong to the category of protein synthesis and/or transport-related protein are the following 29 clones.

BRACE2014306, BRACE3008720, BRAWH3000491, BRCAN2009432, BRHIP2000920, BRTHA3013884, CTONG2013178, HCHON2004531, HLUNG1000017, HLUNG2013851, HSYRA2005496, NT2NE2006909, NT2RI3006340, OCBBF2007068, OCBBF2031167, PUAEN2009795, TBAES2001229, TBAES2004055, TESTI2051867, TESTI4000014, TESTI4000349, TESTI4009608, TESTI4010851, TESTI4034632, TRACH3007479, TRACH3036193, UTERU2017762, UTERU2019940, UTERU2033375

The following 17 clones are also predicted to belong to the category of protein synthesis and/or transport-related protein.

BLADE2000579, BRACE3003053, BRCAN2003070, BRTHA2018011, BRTHA3000296, CTONG2016942, MESAN2005303, NT2RI3002557, NT2RP7008435, PERIC2007068, PLACE7000502, PROST2000452, TESTI4001984, TESTI4017714, THYMU2004284, TRACH3000420, TRACH3007866

The clones predicted to belong to the category of cellular defense-related protein are the following four clones.

BRTHA2015878, CTONG3000084, NT2RI3002842, PEBLM2004666

The following three clones are also predicted to belong to the category of cellular defense-related protein.

BRCAN2002473, NT2RI3007167, TRACH3002561

The clone predicted to belong to the category of development and/or differentiation-related protein is the following one clone.

TESTI4014924

The clones predicted to belong to the category of DNA-binding and/or RNA-binding protein are the following 67 clones.

BRACE2006319, BRACE2047011, BRACE3004150, BRACE3013576, BRACE3024073, BRAMY2030109, BRAWH3005912, BRCAN2002562, BRCOC2003213, BRHIP2021615, BRHIP3008183, BRHIP3025161, BRSTN2004987, BRTHA2018707, BRTHA3016917, CORDB1000140, CTONG1000467, CTONG3000084, CTONG3003972, CTONG3008831, CTONG3009028, FCBBF3013307, FEBRA2026984, HEART2001756, HLUNG2013851, IMR322000127, IMR322000917, KIDNE1000064, NT2NE2006531, NT2RI3003382, NT2RI3007158, NT2RP7000466, NT2RP7004123, OCBBF2036743, OCBBF3009279, PLACE6019385, SPLEN2006122, SPLEN2010912, TESOP2009121, TESTI1000390, TESTI2014716, TESTI2026505, TESTI2040018, TESTI2044796, TESTI2050987, TESTI4007810, TESTI4009374, TESTI4011745, TESTI4012679, TESTI4017001, TESTI4019140, TESTI4019566, TESTI4022716, TESTI4026510, TESTI4034432, TESTI4034912, TESTI4036909, THYMU2035319, THYMU2035735, THYMU3000133, TLIVE2002336, TRACH2023299, TRACH2025749, TRACH3004840, TRACH3036193, UTERU2026025, UTERU3009490

The following 112 clones are also predicted to belong to the category of DNA-binding and/or RNA-binding protein.

BLADE2006830, BRACE2003609, BRACE3001058, BRACE3001113, BRACE3003026, BRACE3003053, BRACE3010076, BRAMY2035070, BRAMY2035449, BRAMY2039341, BRAMY2045471, BRAWH1000369, BRAWH3007441, BRHIP2017553, BRSTN2013354, BRTHA2002133, BRTHA2002702, BRTHA2017364, BRTHA2017972, CERVX2002013, CTONG1000113, CTONG2003348, CTONG2015596, CTONG2020374, CTONG2020378, CTONG2020411, CTONG2024031, CTONG2028758, CTONG3001501, CTONG3004726, DFNES2011192, FCBBF1000509, FCBBF3027854, FEBRA2014122, FEBRA2027609, HCASM2003018, HCASM2003099, HCASM2009424, HCHON2000508, HCHON2000743, HCHON2004858, HSYRA2005628, IMR322013731, MESAN2014412, MESAN2015501, NT2RI2008952, NT2RI2018448, NT2RI2027157, NT2RI3000174, NT2RI3001132, NT2RI3002557, NT2RI3007167, NT2RI3007443, OCBBF2006987, OCBBF2008144, OCBBF2009583, OCBBF2011669, OCBBF2019684, OCBBF2020048, OCBBF2024284, OCBBF2032274, OCBBF2034637, OCBBF3000167, OCBBF3003761, PERIC2007068, SPLEN2016135, SPLEN2016781, SPLEN2036702, STOMA2003158, SYNOV2021953, SYNOV4002744, TESOP2001796, TESOP2005199, TESOP2006398, TESTI2008901, TESTI2026647, TESTI2034251, TESTI2035981, TESTI2037830, TESTI4000155, TESTI4000183, TESTI4000214, TESTI4006473, TESTI4008058, TESTI4008302, TESTI4010382, TESTI4013365, TESTI4014801, TESTI4015442, TESTI4017714, TESTI4025494, TESTI4025547, TESTI4026207, TESTI4028938, TESTI4028958, TESTI4029348, TESTI4031745, TESTI4032090, THYMU2006001, THYMU2028739, THYMU2031139, THYMU3001428, TKIDN2012771, TLIVE2007607, TRACH2007483, TRACH3000134, TRACH3003832, TRACH3007866, UTERU3001053, UTERU3014791, UTERU3017176, TESTI4038779

The clones predicted to belong to the category of ATP binding and/or GTP-binding protein are the following 28 clones.

BRACE3008720, BRACE3009708, BRAMY2047746, BRAMY3004919, BRAWH2014662, BRAWH2016702, BRCAN2009432, BRCAN2024451, BRSTN2013741, BRTHA3008778, BRTHA3009090, CTONG2004062, CTONG2028124, HCHON2004007, OCBBF2037340, SPLEN2030335, TESTI4000288, TESTI4001148, TESTI4002552, TESTI4008429, TESTI4018835, TESTI4021478, TESTI4029836, THYMU2036459, THYMU3001379, TRACH2001549, UTERU3000828, UTERU3001240

The following eight clones are also predicted to belong to the category of ATP binding and/or GTP-binding protein.

BRCAN2014229, BRHIP2008389, CTONG3004550, FEBRA2001990, IMR322013396, IMR322013731, MESAN2001770, TESTI4000319

Although the 208 clones described below have hit data in Pfam, it remains unclear as to which of the above-described categories each of these clones belong. However, if data for proteins having a similar domain or motif are accumulated and their functions clarified in more detail, in the future these clones can be classified into any of the above-described categories. The Clone Name and Functional Domain Name are indicated as “Clone Name//Functional Domain Name”. When a clone had hit data in multiple functional domains, all data were represented, with each marked with a double slash (//).

In addition, even when a clone had multiple hit data in an identical functional domain, these data are fully represented without abridgment.

3NB692002685//R3H domain

3NB692008729//Hr1 repeat motif

ASTRO2003960//F-box domain.

BNGH42003570//EB module//Furin-like cysteine rich region//Thrombospondin type 1 domain

BRACE2010489//LysM domain

BRACE2015314//Bacterial mutT protein

BRACE2016981//Fanconi anaemia group C protein//Bacterial flagellin N-terminus

BRACE2027258//Ank repeat//Ank repeat//Ank repeat//Ank repeat//Ank repeat

BRACE2030341//Kinase associated domain 1

BRACE2035441//Spectrin repeat//Spectrin repeat//Spectrin repeat

BRACE2038329//TS-N domain//UBA domain

BRACE2042550//Thrombospondin type 1 domain//Trypsin Inhibitor like cysteine rich domain//von Willebrand factor type C domain//Thrombospondin type 1 domain

BRACE2044286//CRAL/TRIO domain.//Spectrin repeat

BRACE3000071//Ank repeat//Ank repeat//Ank repeat

BRACE3000973//Leucine Rich Repeat

BRACE3001002//Lipoprotein

BRACE3003192//EGF-like domain//EGF-like domain//EGF-like domain//EGF-like domain//Metallothionein//Keratin, high sulfur B2 protein//EGF-like domain//EGF-like domain//EGF-like domain//EGF-like domain//TB domain//EGF-like domain//EGF-like domain//EGF-like domain//TB domain//EGF-like domain//EGF-like domain

BRACE3004772//SAM domain (Sterile alpha motif)

BRACE3004880//GLTT repeat (12 copies)//GLTT repeat (12 copies)//GLTT repeat (12 copies)//Keratin, high sulfur B2 protein

BRACE3008137//PDZ domain (Also known as DHR or GLGF).//PDZ domain (Also known as DHR or GLGF).//PDZ domain (Also known as DHR or GLGF).//PDZ domain (Also known as DHR or GLGF).

BRACE3008384//Rhomboid family

BRACE3009090//Beige/BEACH domain

BRACE3010397//SCP-like extracellular protein

BRACE3015521//EF hand

BRACE3016884//Keratin, high sulfur B2 protein//Flagellar L-ring protein

BRACE3019084//SAM domain (Sterile alpha motif)

BRAMY2004771//Leucine Rich Repeat//Leucine rich repeat C-terminal domain//Leucine rich repeat N-terminal domain

BRAMY2019300//Leucine Rich Repeat//Leucine rich repeat C-terminal domain//Leucine rich repeat N-terminal domain BRAMY2021498//Thrombospondin type 1 domain//DnaJ central domain (4 repeats)//Thrombospondin type 1 domain//Thrombospondin type 1 domain//Thrombospondin type 1 domain//Thrombospondin type 1 domain

BRAMY2031317//PDZ domain (Also known as DHR or GLGF).

BRAMY2039872//Interferon alpha/beta domain

BRAMY2046989//TPR Domain//TPR Domain//TPR Domain//TPR Domain//TPR Domain//TPR Domain

BRAMY3004224//Leucine rich repeat N-terminal domain//Leucine Rich Repeat//Leucine Rich Repeat//Leucine Rich Repeat//Leucine Rich Repeat//Leucine Rich Repeat//Leucine rich repeat C-terminal domain

BRAMY3005932//Ank repeat

BRAWH1000127//Plexin repeat//Thrombospondin type 1 domain

BRAWH2001395//Myelin basic protein

BRAWH2014954//PDZ domain (Also known as DHR or GLGF).//PDZ domain (Also known as DHR or GLGF).

BRAWH3000078//Adaptin N terminal region//Activin types I and II receptor domain

BRAWH3001891//YCF9

BRAWH3002574//Calpain large subunit, domain III//EF hand

BRAWH3002600//Cadherin domain//Cadherin domain//Cadherin domain

BRAWH3008341//Pentaxin family

BRCAN2002948//Adaptin N terminal region

BRCAN2009203//SAM domain (Sterile alpha motif)

BRCAN2015464//Gag P30 core shell protein

BRCAN2017717//Squash family of serine protease inhibitors

BRCOC2001505//Myelin basic protein

BRCOC2016525//Ank repeat//Ank repeat//Ank repeat//Ank repeat//Ank repeat//Ank repeat//Ank repeat

BRHIP2003786//Ank repeat//Ank repeat//Ank repeat//BTB/POZ domain

BRHIP2005236//Galactose binding lectin domain//Latrophilin Cytoplasmic C-terminal region

BRHIP2007616//Sema domain

BRHIP2009414//Uncharacterized protein family

BRHIP3000339//Myelin basic protein

BRHIP3008313//Ank repeat

BRSTN2001067//Rifin/stevor family

BRTHA2000855//Extracellular link domain

BRTHA2005579//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain//von Willebrand factor type C domain

BRTHA2007122//Ank repeat//Ank repeat//Ank repeat//Ank repeat//SAM domain (Sterile alpha motif)

BRTHA2008527//Leucine Rich Repeat//Leucine Rich Repeat//Leucine Rich Repeat//Leucine Rich Repeat//Leucine Rich Repeat

BRTHA2009311//Vertebrate galactoside-binding lectins

BRTHA2010884//Thrombospondin type 1 domain//CUB domain

BRTHA2013262//Keratin, high sulfur B2 protein

BRTHA2014792//SET domain

BRTHA2015406//UBA domain

BRTHA2016496//Peptidase C13 family

BRTHA2018591//GTPase of unknown function

BRTHA2018624//Galactose binding lectin domain//Activin types I and II receptor domain//Galactose binding lectin domain

BRTHA2019048//Domain of unknown function DUF71

BRTHA3003074//Fanconi anaemia group C protein

BRTHA3008310//Homeobox domain

CTONG1000341//EGF-like domain//EGF-like domain//Metallothionein//EGF-like domain//EB module//EGF-like domain//EGF-like domain//EGF-like domain

CTONG2001877//MutT-like domain

CTONG2008233//DnaJ domain

CTONG2017500//F-box domain.

CTONG2020026//Herpesvirus VP23 like capsid protein

CTONG2028687//TPR Domain//TPR Domain

CTONG3000686//TPR Domain//TPR Domain//TPR Domain//TPR Domain

CTONG3004072//Beta type Zein//Keratin, high sulfur B2 protein

CTONG3006067//DnaJ central domain (4 repeats)

CTONG3006186//PDZ domain (Also known as DHR or GLGF)//Apolipoprotein A1/A4/E family//WW domain

CTONG3009385//TPR Domain//TPR Domain//TPR Domain//TPR Domain//TPR Domain//TPR Domain//TPR Domain//TPR Domain//TPR Domain//TPR Domain

DFNES2000146//Plexin repeat//Thrombospondin type 1 domain

DFNES2005266//Thrombospondin type 1 domain

FCBBF3009888//Keratin, high sulfur B2 protein//u-PAR/Ly-6 domain

FCBBF3012170//Thrombospondin type 1 domain

FEBRA2000253//Flagellar L-ring protein

FEBRA2007801//IBR domain

FEBRA2021571//von Willebrand factor type D domain

FEBRA2024150//DENN (AEX-3) domain

HCHON2004776//Protein of unknown function DUF93

HEART1000139//Troponin

HEART2006909//CBS domain//CBS domain

HEART2010495//Tau and MAP proteins, tubulin-binding

HLUNG2000014//Lectin C-type domain

HLUNG2002958//EF hand

HLUNG2011298//Oxidoreductase FAD/NAD-binding domain

IMR322006495//Tropomyosins

KIDNE2000846//Sodium:neurotransmitter symporter family

KIDNE2001361//Domain of unknown function DUF19

KIDNE2011635//Sodium:solute symporter family

KIDNE2012945//CUB domain//Pentaxin family

NESOP2001656//Polyomavirus coat protein

NT2RI2008724//GGL domain

NT2RI2025909//Mitochondrial carrier proteins//Mitochondrial carrier proteins//Mitochondrial carrier proteins

NT2RI2025957//PDZ domain (Also known as DHR or GLGF).

NT2RI3007543//DnaJ domain

NT2RP7000359//FERM domain (Band 4.1 family)//Insulin-like growth factor binding proteins//PDZ domain (Also known as DHR or GLGF).

NT2RP7004027//CUB domain//Sushi domain (SCR repeat)

NT2RP7011570//Gag P30 core shell protein

NT2RP8000296//BTB/POZ domain//Kelch motif//Kelch motif//Kelch motif//Kelch motif//Kelch motif//Kelch motif

NTONG2005277//Ank repeat//Ank repeat//Ank repeat//Ank repeat//Ank repeat//Ank repeat//Ank repeat//Ank repeat

NTONG2006354//Ank repeat

NTONG2007517//BTB/POZ domain

OCBBF2006764//Sushi domain (SCR repeat)//CUB domain//Sushi domain (SCR repeat)//CUB domain//Sushi domain (SCR repeat)

OCBBF2010416//Major intrinsic protein

OCBBF2020838//Fork head domain

OCBBF2021323//Regulatory subunit of type II PKA R-subunit

OCBBF2033869//CUB domain

PERIC2001228//Leucine Rich Repeat//Leucine Rich Repeat//Leucine Rich Repeat//Leucine Rich Repeat//Leucine Rich Repeat//Leucine Rich Repeat//Leucine Rich Repeat

PERIC2003720//Ezrin/radixin/moesin family

PLACE6020031//Ank repeat//Ank repeat

PLACE7000514//Filamin/ABP280 repeat.

PROST2018090//Sushi domain (SCR repeat)//Sushi domain (SCR repeat)//Chitin binding Peritrophin-A domain//HYR domain//Sushi domain (SCR repeat)

RECTM2000433//Jacalin-like lectin domain

SKMUS2006394//Ank repeat//Ank repeat//Ank repeat//Ank repeat//Ank repeat//Ank repeat//Ank repeat

SMINT1000192//Small hydrophilic plant seed proteins

SPLEN2002147//Phosphatidylinositol transfer protein

SPLEN2002467//DB module//F-box domain.//Leucine Rich Repeat

SPLEN2031780//Domain of unknown function DUF139//Domain of unknown function DUF139

SPLEN2034081//Insulin-like growth factor binding proteins

SPLEN2036821//Mitochondrial carrier proteins

SYNOV2005448//Apidaecin

SYNOV2005817//Domain of unknown function DUF19//Tissue factor

SYNOV2006430//Nitrogen regulatory protein P-II

SYNOV2014400//EGF-like domain//Granulins//Granulins//EGF-like domain

SYNOV4007553//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine rich repeat C-terminal domain//TIR domain

SYNOV4008440//Adaptin N terminal region

TESOP2001953//Leucine Rich Repeat//Leucine Rich Repeat//Leucine Rich Repeat//Leucine Rich Repeat//Leucine Rich Repeat//Leucine Rich Repeat//Leucine Rich Repeat

TESTI2000443//Leucine Rich Repeat//Leucine Rich Repeat//Leucine Rich Repeat

TESTI2004700//Leucine Rich Repeat//Leucine Rich Repeat//Leucine Rich Repeat//Leucine Rich Repeat

TESTI2027019//Leucine Rich Repeat

TESTI4000462//Keratin, high sulfur B2 protein

TESTI4000970//Ezrin/radixin/moesin family

TESTI4002491//NSF attachment protein

TESTI4006546//Tudor domain//Tudor domain//Tudor domain

TESTI4007064//DENN (AEX-3) domain//PPR repeat//LIM domain containing proteins

TESTI4011484//SAM domain (Sterile alpha motif)

TESTI4012406//Kringle domain

TESTI4015471//Tropomyosins

TESTI4016110//DnaJ domain

TESTI4017137//Keratin, high sulfur B2 protein

TESTI4017575//MSP (Major sperm protein) domain

TESTI4018152//FERM domain (Band 4.1 family)

TESTI4018555//Granulins

TESTI4020092//Laminin G domain

TESTI4023555//Lectin C-type domain

TESTI4025920//Adaptin N terminal region

TESTI4026192//Domain of unknown function

TESTI4027557//Vertebrate galactoside-binding lectins//Vertebrate galactoside-binding lectins

TESTI4028429//WAP-type (Whey Acidic Protein) ‘four-disulfide core’

TESTI4028612//Major intrinsic protein

TESTI4028983//Serum amyloid A protein

TESTI4030505//Metallothionein family 5

TESTI4038492//Serum amyloid A protein

TESTI4039659//DnaJ domain

TESTI4041053//Ank repeat//Ank repeat//Ank repeat//Ank repeat//Armadillo/beta-catenin-like repeats//Armadillo/beta-catenin-like repeats//Armadillo/beta-catenin-like repeats//Armadillo/beta-catenin-like repeats//Armadillo/beta-catenin-like repeats//Armadillo/beta-catenin-like repeats

TESTI4044084//Domain of unknown function

TESTI4046487//Hantavirus nucleocapsid protein

TESTI4046819//Metallothionein//PTS HPr component phosphorylation sites

THYMU2004693//CX module

THYMU2011736//EGF-like domain//EGF-like domain//EB module//EGF-like domain//TB domain//EGF-like domain//EGF-like domain

THYMU2016204//Metallothionein

THYMU2027734//Parvovirus coat protein VP2

THYMU2038369//Regulatory subunit of type II PKA R-subunit

THYMU2038797//Lectin C-type domain

THYMU3000028//Zona pellucida-like domain

THYMU3003212//Cytidine and deoxycytidylate deaminase zinc-binding region

THYMU3003763//Leucine rich repeat N-terminal domain//Polyomavirus coat protein

THYMU3007137//PDZ domain (Also known as DHR or GLGF).//PDZ domain (Also known as DHR or GLGF).

THYMU3008171//TPR Domain

TLIVE2002338//Transforming growth factor beta like domain

TLIVE2002690//von Willebrand factor type D domain

TLIVE2003225//CUB domain//Sushi domain (SCR repeat)//CUB domain//Sushi domain (SCR repeat)

TLIVE2008229//TPR Domain//TPR Domain

TRACH2001443//TIR domain

TRACH3001427//UBX domain

TRACH3003379//Protein phosphatase 2A regulatory B subunit

TRACH3008713//NSF attachment protein

TRACH3035235//S-100/ICaBP type calcium binding domain

TUTER2000425//KRAB box

UTERU1000031//ENTH domain//VHS domain

UTERU2006115//Adaptin N terminal region

UTERU2006568//IBR domain

UTERU2019706//TCP-1/cpn60 chaperonin family//TCP-1/cpn60 chaperonin family

UTERU2035328//WW domain//WW domain//WW domain//FF domain//FF domain//FF domain

UTERU2035331//Fibrillar collagen C-terminal domain

UTERU2035452//EGF-like domain//Metallothionein//EGF-like domain

UTERU3001652//Wiskott Aldrich syndrome homology region 2

UTERU3001766//Apidaecin

UTERU3001988//TPR Domain

UTERU3002667//Polyomavirus coat protein

UTERU3003178//TPR Domain//TPR Domain//TPR Domain//TPR Domain//PPR repeat

UTERU3005585//PDZ domain (Also known as DHR or GLGF).

UTERU3007640//NSF attachment protein

UTERU3008660//TPR Domain//TPR Domain

UTERU3009871//Ank repeat//Ank repeat//Ank repeat//Ank repeat//TPR Domain//Ank repeat//Ank repeat

UTERU3009979//EGF-like domain//EGF-like domain//EGF-like domain//Trypsin Inhibitor like cysteine rich domain//EGF-like domain//Laminin G domain//Thrombospondin N-terminal -like domains//Laminin G domain

UTERU3015500//Leucine rich repeat N-terminal domain//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat//Leucine Rich Repeat

Likewise, although the 45 clones described below have hit data in Pfam (see Example 5), it remains unclear as to which of the above-described categories each of the clones belong. However, if data for proteins comprising a similar domain or motif are accumulated and their functions are clarified in more detail, in the future these clones can be classified into any of the categories described above.

3NB692004724//KRAB box//Integrase core domain

ADRGL2000042//Nucleosome assembly protein (NAP)

BRACE2037299//Integrase core domain

BRALZ2017844//Homeobox domain

BRAWH2006207//KRAB box

BRCAN2002854//SAP domain

BRHIP2006617//TPR Domain//TPR Domain

BRHIP2012360//XPG N-terminal domain//XPG I-region

BRHIP3008314//Sir2 family

BRTHA2016318//KE2 family protein

CTONG2019822//Hepatitis C virus core protein

FCBBF3010361//Fork head domain

FEBRA2006519//Thrombospondin type 1 domain//Thrombospondin type 1 domain

FEBRA2028256//EGF-like domain//EGF-like domain//EGF-like domain//EGF-like domain//EGF-like domain//TB domain//EGF-like domain//EGF-like domain//EGF-like domain//EGF-like domain//EB module//Squash family of serine protease inhibitors//EGF-like domain//EGF-like domain

FEBRA2028516//GRIP domain

HCASM2008536//XRCC1 N terminal domain

IMR322007078//UBA domain

IMR322008651//Helix-hairpin-helix motif.

LIVER2000247//Sodium

OCBBF2003327//Thrombospondin type 1 domain//Thrombospondin type 1 domain//Thrombospondin type 1 domain

PROST2009320//LIM domain containing proteins//LIM domain containing proteins

PUAEN2006335//Formin Homology 2 Domain

SKMUS2003194//SAP domain

SPLEN2039379//Transthyretin precursor (formerly prealbumin)

SYNOV1000256//Leucine Rich Repeat//BAH domain//Leucine Rich Repeat//Leucine Rich Repeat//Leucine Rich Repeat

SYNOV2006620//Nuclear transition protein 2

SYNOV4003981//Somatomedin B domain//WAP-type (Whey Acidic Protein) ‘four-disulfide core’//Hemopexin//Hemopexin

SYNOV4005889//Apolipoprotein A1/A4/E family

TESOP2006865//KRAB box

TESTI1000266//Integrase core domain

TESTI2050780//Kazal-type serine protease inhibitor domain

TESTI4000137//Domain of unknown function

TESTI4024387//GDP dissociation inhibitor

TESTI4029528//RanBP1 domain.

TESTI4038721//Squash family of serine protease inhibitors

TESTI4046240//Sir2 family

THYMU2035078//Domain of unknown function DUF27

THYMU3000269//FAD binding domain

THYMU3000360//Integrase core domain

TRACH1000212//TSC-22/dip/bun family

TRACH2000862//Guanylate-binding protein

TRACH2019672//CRAL/TRIO domain.

TRACH2024559//IQ calmodulin-binding motif//IQ calmodulin-binding motif

UTERU2032279//Serpins (serine protease inhibitors)

UTERU2033577//KRAB box

In addition, when data for proteins are accumulated and novel domains and motifs are found, in the future the remaining clones, which had no hit data in the search with Pfam, can be classified into any of the above-described categories if a new functional domain or motif is identified by re-analyzing the deduced amino acid sequences of the clones using a homology search against an updated database.

### EXAMPLE 8

### Expression Frequency Analysis In Silico

The cDNA libraries derived from various tissues and cells as indicated in Example 1 were prepared, and cDNA clones were selected from each library at random. The 5′-end sequences were determined and the database was constructed based on the data. The database was constructed based on the nucleotide sequences of 1,402,069 clones, and thus the population of the database is large enough for the analysis.

Then, clones having a homologous sequence are categorized into a single cluster (clustering) by searching the nucleotide sequences of respective clones in this database with the program of nucleotide sequence homology search; the number of clones belonging to each cluster was determined and normalized for every library; thus, the ratio of a certain gene in each cDNA library was determined. This analysis gave the information of the expression frequency of genes in tissues and cells which were sources of the cDNA libraries.

Then, in order to analyze the expression of a gene containing the nucleotide sequence of the cDNA of the present invention in tissues and cells, the library derived from a tissue or a cell used in the large-scale cDNA analysis was subjected to the comparison of the expression levels between tissues or cells. Namely, the expression frequency was analyzed by comparing the previously normalized values between tissues and/or cells for which the nucleotide sequences of 600 or more cDNA clones had been analyzed. By this analysis, some of the genes were revealed to be involved in the pathology and functions indicated below. Each value in Tables 2 to 24 shown below represents a relative expression frequency; the higher the value, the higher the expression level. The genes which are included a part of the Tables indicate not so big difference between compared libraries, but when compared with other libraries from another tissue or cell based on Example 9, they indicate significant difference. Thus, the genes are specific in each tissue or cell, and can be considered to be useful as diagnosing markers for the disease as well as useful for analyzing molecular mechanisms.

Osteoporosis-Related Genes

Osteoporosis is a pathology in which bones are easily broken owing to overall decrease in components of bone. The onset involves the balance between the functions of osteoblast producing bone and osteoclast absorbing bone, namely bone metabolism. Thus, the genes involved in the increase of osteoclasts differentiating from precursor cells of monocyte/macrophage line (Molecular Medicine 38. 642-648. (2001)) are genes involved in osteoporosis relevant to bone metabolism.

A nucleotide sequence information-based analysis was carried out to identify the genes whose expression frequencies are higher or lower in CD34+ cell (cell expressing a glycoprotein CD34) treated with the osteoclast differentiation factor (Molecular Medicine 38. 642-648. (2001)) than in the untreated CD34+ cell, which is the precursor cell of monocyte/macrophage line. The result of comparative analysis for the frequency between the two cDNA libraries prepared from the RNA of CD34+ cells (CD34C) and from the RNA of CD34+ cells treated with the osteoclast differentiation factor (D30ST, D60ST or D90ST) showed that the genes whose expression levels were different between the two were the following 15 and 2 clones (Table 2).

BRACE3013780, BRAMY2047420, BRSTN2016470, CTONG3008894, D3OST2002182, D3OST2002648, D3OST3000169, PEBLM2005183, PUAEN2009655, TESTI4000014, TESTI4010851, TRACH2023299, TRACH2025535, TRACH3001427, UTERU2006137

HCHON2000508, TESTI2015626

These genes are involved in osteoporosis.

Genes Involved in Neural Cell Differentiation

Genes involved in neural cell differentiation are useful for treating neurological diseases. Genes with varying expression levels in response to induction of cellular differentiation in neural cells are thought to be involved in neurological diseases.

A survey was performed for genes whose expression levels are varied in response to induction of differentiation (stimulation by retinoic acid (RA) or growth inhibitor treatment after RA stimulation) in cultured cells of a neural strain, NT2. The result of comparative analysis of cDNA libraries derived from undifferentiated NT2 cells (NT2RM) and the cells subjected to the differentiation treatment (NT2RP, NT2RI or NT2NE) showed that the genes whose expression levels were different between the two were the following 174 and 30 clones (Table 3).

BNGH42007788, BRACE1000186, BRACE2006319, BRACE2014306, BRACE2015058, BRACE2044286, BRACE3010428, BRAMY2044078, BRAWH2014645, BRAWH2014662, BRAWH3002574, BRAWH3003992, BRAWH3005981, BRAWH3007592, BRCAN2009432, BRCAN2016619, BRCAN2028355, BRHIP2001074, BRHIP2007741, BRHIP2014228, BRHIP2024146, BRHIP3007586, BRHIP3018797, BRTHA2003461, BRTHA3000633, BRTHA3003490, COLON2001721, CTONG1000087, CTONG2008233, CTONG2020638, CTONG2028124, CTONG3003905, CTONG3008894, CTONG3009028, CTONG3009239, DFNES2011499, FCBBF3001977, FEBRA1000030, FEBRA2006396, FEBRA2007801, HCHON2000028, HCHON2000244, HCHON2001084, HCHON2001217, HCHON2001548, HCHON2006250, HEART1000074, HHDPC1000118, HSYRA2009075, IMR322000127, IMR322001380, KIDNE2000665, KIDNE2002252, MESAN2006563, MESAN2012054, MESAN2015515, NT2NE2003252, NT2NE2005890, NT2NE2006531, NT2NE2006909, NT2NE2008060, NT2RI2003993, NT2RI2004618, NT2RI2005166, NT2RI2006686, NT2RI2008724, NT2RI2009855, NT2RI2011422, NT2RI2011683, NT2RI2012659, NT2RI2012990, NT2RI2013357, NT2RI2014247, NT2RI2014551, NT2RI2014733, NT2RI2016128, NT2RI2018311, NT2RI2018883, NT2RI2019751, NT2RI2023303, NT2RI2025909, NT2RI2025957, NT2RI2027081, NT2RI2027396, NT2RI3000622, NT2RI3001263, NT2RI3001515, NT2RI3002303, NT2RI3002842, NT2RI3002892, NT2RI3003031, NT2RI3003095, NT2RI3003162, NT2RI3003382, NT2RI3003409, NT2RI3004381, NT2RI3004510, NT2RI3005202, NT2RI3005403, NT2RI3005724, NT2RI3006132, NT2RI3006171, NT2RI3006284, NT2RI3006340, NT2RI3006376, NT2RI3006673, NT2RI3006796, NT2RI3007065, NT2RI3007158, NT2RI3007291, NT2RI3007543, NT2RI3007757, NT2RI3007978, NT2RI3008055, NT2RI3008162, NT2RI3008652, NT2RI3008697, NT2RI3008974, NT2RI3009158, NT2RP7000359, NT2RP7000466, NT2RP7004027, NT2RP7004123, NT2RP7005118, NT2RP7005529, NT2RP7005846, NT2RP7009030, NT2RP7009147, NT2RP7009867, NT2RP7010128, NT2RP7010599, NT2RP7011570, NT2RP7013795, NT2RP7014005, NT2RP7015512, NT2RP7017365, NT2RP7017474, NT2RP7017546, NT2RP8000137, NT2RP8000296, NT2RP8000483, NTONG2005969, OCBBF2007028, OCBBF2037068, PLACE7000514, PUAEN2007044, SPLEN2002467, SPLEN2006122, SPLEN2028914, SPLEN2031547, SYNOV4002346, SYNOV4007671, SYNOV4008440, TESOP2002273, TESTI2003573, TESTI4000014, TESTI4009286, TESTI4010851, TESTI4012702, TESTI4029671, TESTI4037156, THYMU3000133, TRACH1000205, TRACH2005811, TRACH2007834, TRACH2025535, TRACH3001427, TRACH3002192, TRACH3004721, TRACH3008093, TRACH3008535, TRACH3008713, UTERU2002410, UTERU2023175

ADRGL2000042, BRACE2003609, BRACE3003026, BRHIP3000017, CTONG2020411, FCBBF1000509, FCBBF3027854, FEBRA2028516, HCHON2000508, IMR322001879, NT2RI2005772, NT2RI2008952, NT2RI2009583, NT2RI2018448, NT2RI2027157, NT2RI3000174, NT2RI3001132, NT2RI3002557, NT2RI3005928, NT2RI3007167, NT2RI3007443, NT2RP7008435, NT2RP8000521, OCBBF2006987, PERIC2007068, TESTI2015626, TESTI4015442, TLIVE2002046, TRACH3000134, TUTER2000057

These genes are neurological disease-related genes.

Genes Involved in Alzheimer's Disease

Alzheimer's disease is a cranial neurological disease that is characterized by memory loss. As the disease advances, patients can no longer support themselves and require nursing. Alzheimer's disease eventually leads to atrophication of the brain itself. Environmental factors such as stress, and vascular factors such as hypertension and cholesterolemia, are assumed, but not confirmed, to contribute to the onset of Alzheimer's disease. Genes whose expression levels differ between normal brain tissues and tissues affected with Alzheimer's disease are expected to be involved in Alzheimer's disease. Such genes can be used to elucidate the disease's onset mechanism and in genetic diagnosis. cDNA libraries derived from the cerebral cortex of Alzheimer patients (BRALZ and BRASW), and a library derived from whole tissues of a normal brain (BRAWH) were analyzed and compared (Table 4). The results showed that genes whose expression levels differed between the two are the following 250 and 41 clones listed below.

ASTRO1000009, BLADE2008398, BRACE1000186, BRACE1000258, BRACE1000533, BRACE2005457, BRACE2010489, BRACE2014657, BRACE2035381, BRACE2044286, BRACE2045954, BRACE3000787, BRACE3003192, BRACE3005499, BRACE3007480, BRACE3009237, BRACE3009724, BRACE3009747, BRACE3010428, BRACE3011271, BRACE3011421, BRACE3012364, BRACE3022769, BRACE3026735, BRACE3031838, BRALZ2011796, BRALZ2012183, BRALZ2012848, BRALZ2014484, BRALZ2016085, BRALZ2016498, BRALZ2017359, BRAMY2003008, BRAMY2005052, BRAMY2019300, BRAMY2019963, BRAMY2036567, BRAMY2037823, BRAMY2040592, BRAMY3002803, BRAMY3004224, BRAMY3005091, BRASW1000053, BRASW1000125, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCOC2003213, BRCOC2014033, BRCOC2020142, BRHIP2000920, BRHIP2005719, BRHIP2007741, BRHIP2014228, BRHIP2024146, BRHIP2026288, BRHIP3000339, BRHIP3006683, BRHIP3007586, BRHIP3008405, BRHIP3018797, BRSSN2000684, BRSSN2011738, BRSSN2014299, BRSTN2008052, BRSTN2015015, BRSTN2016470, BRTHA1000311, BRTHA2008335, BRTHA3002427, BRTHA3003490, BRTHA3008520, BRTHA3017848, COLON2001721, CTONG2017500, CTONG2028124, CTONG3000657, CTONG3001123, CTONG3009328, FCBBF2001183, FCBBF3001977, FEBRA2007544, FEBRA2007801, FEBRA2020886, FEBRA2028618, HCASM2007047, HCHON2000244, HCHON2000626, HCHON2001217, HCHON2002676, HCHON2006250, HEART1000074, HHDPC1000118, HLUNG2002465, IMR322000127, IMR322001380, IMR322002035, KIDNE2006580, MESAN2006563, MESAN2012054, MESTC1000042, NOVAR2001783, NT2NE2006909, NT2RI2008724, NT2RI2012659, NT2RI2014733, NT2RI3002892, NT2RI3006284, NT2RI3006673, NT2RI3007543, NT2RI3008055, NT2RP7005529, NT2RP7009147, NT2RP7014005, NT2RP7017474, NTONG2005969, OCBBF2001794, OCBBF2006005, OCBBF2006764, OCBBF2007028, OCBBF2007114, OCBBF2010140, OCBBF2021286, OCBBF2023162, OCBBF2024850, OCBBF2028935, OCBBF2036743, OCBBF2038317, OCBBF3000483, OCBBF3008230, PEBLM2004666, PLACE6001185, PUAEN2005930, PUAEN2006701, PUAEN2007044, PUAEN2009655, SMINT2001818, SPLEN2028914, SPLEN2031424, SPLEN2031547, SPLEN2034781, SPLEN2036932, SYNOV2014400, SYNOV4002346, SYNOV4002883, SYNOV4007430, SYNOV4007671, SYNOV4008440, TESOP2002273, TESOP2002451, TESTI4000014, TESTI4000209, TESTI4001100, TESTI4006137, TESTI4008797, TESTI4009286, TESTI4010851, TESTI4013817, TESTI4014694, TESTI4021478, TESTI4022936, TESTI4024420, TESTI4027821, THYMU2001090, THYMU2033308, THYMU2035735, THYMU2039315, THYMU3001234, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3001427, TRACH3002192, TRACH3004068, TRACH3004721, TRACH3005294, TRACH3007479, TRACH3008093, TRACH3009455, UTERU2005621, UTERU2006115, UTERU2019706, UTERU2023039, UTERU2026203, UTERU3005230, UTERU3007640, UTERU3009871

ADRGL2000042, BLADE2006830, BRACE2003609, BRALZ2017844, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRHIP3026052, BRTHA2018443, BRTHA3003000, CTONG2020374, CTONG2020378, CTONG2024031, FCBBF1000509, FEBRA2001990, FEBRA2006519, FEBRA2028516, HCHON2000743, IMR322001879, NT2RI2009583, OCBBF2008144, PERIC2007068, PUAEN2006335, SPLEN2039379, TESTI4001984, TESTI4008058, TESTI4025268, TESTI4032090, THYMU3000360, TLIVE2002046, TRACH3000134, UTERU2021820, UTERU2028734

These genes are involved in Alzheimer's disease.

Genes Involved in Parkinson's Disease

Parkinson's disease is a cranial neurological disease characterized by impaired production of the neurotransmitter dopamine in the substantia nigra in the brain. This results in dyskinesia, such as hand tremors, and impaired body movement due to muscular rigidity. Normally, the number of brain neurons gradually decreases with age. However, compared to healthy people, patients with Parkinson's disease experience a rapid and marked decrease in the number of neurons in their substantia nigra. Genes whose expression levels differ between tissues of the whole brain and the nigra are expected to be involved in Parkinson's disease. These genes exhibit nigra-specific alterations in their expression levels, and can be used to elucidate the disease onset mechanism and in gene diagnosis. cDNA libraries derived from the substantia nigra (BRSSN) and a library derived from whole tissues of a normal brain (BRAWH) were analyzed and compared (Table 5). Genes whose expression levels differed between the two were the 250 clones and 40 clones listed below.

ASTRO1000009, BLADE2008398, BRACE1000186, BRACE1000258, BRACE1000533, BRACE2005457, BRACE2010489, BRACE2014657, BRACE2035381, BRACE2044286, BRACE2045954, BRACE3000787, BRACE3003192, BRACE3005499, BRACE3007480, BRACE3009237, BRACE3009724, BRACE3009747, BRACE3010428, BRACE3011271, BRACE3011421, BRACE3012364, BRACE3013780, BRACE3022769, BRACE3026735, BRACE3031838, BRALZ2011796, BRAMY2003008, BRAMY2005052, BRAMY2019300, BRAMY2019963, BRAMY2036567, BRAMY2037823, BRAMY2040592, BRAMY2047420, BRAMY3002803, BRAMY3004224, BRAMY3005091, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCOC2003213, BRCOC2014033, BRCOC2020142, BRHIP2000920, BRHIP2005719, BRHIP2007741, BRHIP2014228, BRHIP2024146, BRHIP3000339, BRHIP3006683, BRHIP3007586, BRHIP3008405, BRHIP3018797, BRSSN2000684, BRSSN2003086, BRSSN2004496, BRSSN2004719, BRSSN2006892, BRSSN2008549, BRSSN2008797, BRSSN2011262, BRSSN2011738, BRSSN2013874, BRSSN2014299, BRSSN2014424, BRSSN2014556, BRSSN2018581, BRSSN2018925, BRSTN2008052, BRSTN2015015, BRSTN2016470, BRTHA1000311, BRTHA2003461, BRTHA2008335, BRTHA3002427, BRTHA3003490, BRTHA3008520, BRTHA3017848, COLON2001721, CTONG2017500, CTONG2028124, CTONG3000657, CTONG3001123, CTONG3009328, FCBBF2001183, FCBBF3001977, FEBRA2007544, FEBRA2007801, FEBRA2020886, FEBRA2024136, FEBRA2025427, FEBRA2028618, HCASM2007047, HCHON2000244, HCHON2000626, HCHON2001217, HCHON2002676, HCHON2006250, HEART1000074, HHDPC1000118, HLUNG2002465, IMR322000127, IMR322002035, KIDNE2006580, MESAN2006563, MESAN2012054, MESTC1000042, NOVAR2001783, NT2NE2006909, NT2RI2008724, NT2RI2012659, NT2RI2014733, NT2RI3002892, NT2RI3006284, NT2RI3006673, NT2RI3007543, NT2RI3008055, NT2RP7005529, NT2RP7009147, NT2RP7014005, NT2RP7017474, OCBBF2001794, OCBBF2006005, OCBBF2006764, OCBBF2007028, OCBBF2010140, OCBBF2021286, OCBBF2024850, OCBBF2028935, OCBBF2036743, OCBBF2038317, OCBBF3000483, OCBBF3008230, PEBLM2004666, PLACE6001185, PUAEN2005930, PUAEN2006701, PUAEN2007044, PUAEN2009655, SPLEN2028914, SPLEN2031424, SPLEN2031547, SPLEN2034781, SPLEN2036932, SYNOV2014400, SYNOV4002346, SYNOV4002883, SYNOV4007430, SYNOV4008440, TESOP2002451, TESTI4000014, TESTI4000209, TESTI4001100, TESTI4006137, TESTI4008797, TESTI4009286, TESTI4010851, TESTI4013817, TESTI4014694, TESTI4021478, TESTI4022936, TESTI4024420, TESTI4027821, TESTI4037156, THYMU2001090, THYMU2033308, THYMU2035735, THYMU2039315, THYMU3001234, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3001427, TRACH3002192, TRACH3004721, TRACH3005294, TRACH3007479, TRACH3008093, TRACH3009455, UTERU2006115, UTERU2019706, UTERU2023039, UTERU2026203, UTERU3005230, UTERU3007640, UTERU3009871

ADRGL2000042, BLADE2006830, BRACE2003609, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRTHA2018443, BRTHA3003000, CTONG2020374, CTONG2020378, CTONG2024031, FCBBF1000509, FEBRA2001990, FEBRA2006519, FEBRA2028516, HCHON2000743, IMR322001879, NT2RI2009583, OCBBF2008144, PERIC2007068, PUAEN2006335, SPLEN2039379, TESTI2015626, TESTI4001984, TESTI4008058, TESTI4025268, TESTI4032090, THYMU3000360, TLIVE2002046, TRACH3000134, UTERU2021820, UTERU2028734

These genes are involved in Parkinson's disease.

Genes Involved in Short-Term Memory and Dementia

In the brain, the hippocampus is a highly important memory-related area. The hippocampus functions to establish a memory by judging whether acquired information is necessary, and then accumulating the memory in another area of the brain. According to clinical findings, patients can retain a new memory for only about five minutes with an abnormal, or at the worst without a hippocampus. Some dementia patients are presumed to have hippocampus abnormalities. Thus, genes whose expression levels differ between tissues of the whole brain and the hippocampus are expected to be involved in memory or dementia. Such genes can be used to elucidate the mechanism underlying the memory and in gene diagnosis. cDNA libraries derived from the hippocampus (BRHIP) and from the whole tissues of a normal brain (BRAWH) were analyzed and compared (Table 6). Genes whose expression levels differed between the two were the 370 clones and 59 clones listed below.

ASTRO1000009, BLADE2001371, BLADE2008398, BNGH42007788, BRACE1000186, BRACE1000258, BRACE1000533, BRACE2005457, BRACE2010489, BRACE2014657, BRACE2015058, BRACE2018762, BRACE2030341, BRACE2035381, BRACE2044286, BRACE2045954, BRACE3000787, BRACE3003192, BRACE3005499, BRACE3007480, BRACE3009237, BRACE3009724, BRACE3009747, BRACE3010428, BRACE3011271, BRACE3011421, BRACE3012364, BRACE3018963, BRACE3022769, BRACE3026735, BRACE3031838, BRALZ2011796, BRAMY2003008, BRAMY2005052, BRAMY2019300, BRAMY2019963, BRAMY2031317, BRAMY2036567, BRAMY2037823, BRAMY2040592, BRAMY2044078, BRAMY3002803, BRAMY3004224, BRAMY3005091, BRAMY3009811, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2020710, BRCAN2028355, BRCOC2003213, BRCOC2014033, BRCOC2020142, BRHIP2000691, BRHIP2000819, BRHIP2000826, BRHIP2000920, BRHIP2001074, BRHIP2001805, BRHIP2001927, BRHIP2002122, BRHIP2002172, BRHIP2002346, BRHIP2003242, BRHIP2003786, BRHIP2003917, BRHIP2004312, BRHIP2004359, BRHIP2004814, BRHIP2004883, BRHIP2005236, BRHIP2005354, BRHIP2005600, BRHIP2005719, BRHIP2005752, BRHIP2005932, BRHIP2006800, BRHIP2007616, BRHIP2007741, BRHIP2009340, BRHIP2009414, BRHIP2009474, BRHIP2013699, BRHIP2014228, BRHIP2021615, BRHIP2022221, BRHIP2024146, BRHIP2024165, BRHIP2026061, BRHIP2026288, BRHIP2029176, BRHIP2029393, BRHIP3000339, BRHIP3000526, BRHIP3001283, BRHIP3006683, BRHIP3007483, BRHIP3007586, BRHIP3008183, BRHIP3008313, BRHIP3008344, BRHIP3008405, BRHIP3008565, BRHIP3008598, BRHIP3008997, BRHIP3009099, BRHIP3009448, BRHIP3011241, BRHIP3013765, BRHIP3013897, BRHIP3015751, BRHIP3016213, BRHIP3018797, BRHIP3020182, BRHIP3024118, BRHIP3024533, BRHIP3024725, BRHIP3025161, BRHIP3025702, BRHIP3026097, BRHIP3027137, BRHIP3027854, BRSSN2000684, BRSSN2004719, BRSSN2008549, BRSSN2011738, BRSSN2014299, BRSTN2008052, BRSTN2015015, BRSTN2016470, BRSTN2018083, BRTHA1000311, BRTHA2002442, BRTHA2008335, BRTHA3000297, BRTHA3001721, BRTHA3002427, BRTHA3003490, BRTHA3005046, BRTHA3008520, BRTHA3008778, BRTHA3009090, BRTHA3015910, BRTHA3017848, COLON2001721, CTONG1000087, CTONG1000088, CTONG1000467, CTONG2000042, CTONG2008233, CTONG2009423, CTONG2017500, CTONG2019788, CTONG2028124, CTONG3000657, CTONG3001123, CTONG3001370, CTONG3002412, CTONG3004072, CTONG3008894, CTONG3009239, CTONG3009328, DFNES2011499, FCBBF2001183, FCBBF3001977, FEBRA2000253, FEBRA2007544, FEBRA2007801, FEBRA2008287, FEBRA2010719, FEBRA2020886, FEBRA2028618, HCASM2007047, HCHON2000028, HCHON2000244, HCHON2000626, HCHON2001217, HCHON2002676, HCHON2005921, HCHON2006250, HEART1000074, HEART2007031, HHDPC1000118, HLUNG2002465, HLUNG2003003, IMR322000127, IMR322001380, IMR322002035, KIDNE2005543, KIDNE2006580, MESAN2006563, MESAN2012054, MESTC1000042, NOVAR2001783, NT2NE2006909, NT2RI2008724, NT2RI2012659, NT2RI2014733, NT2RI2018311, NT2RI3001515, NT2RI3002892, NT2RI3004510, NT2RI3005724, NT2RI3006284, NT2RI3006673, NT2RI3007291, NT2RI3007543, NT2RI3008055, NT2RP7005529, NT2RP7009147, NT2RP7014005, NT2RP7017474, OCBBF2001794, OCBBF2003819, OCBBF2006005, OCBBF2006151, OCBBF2006764, OCBBF2007028, OCBBF2007068, OCBBF2010140, OCBBF2020741, OCBBF2021286, OCBBF2024719, OCBBF2024850, OCBBF2028935, OCBBF2036743, OCBBF2038317, OCBBF3000296, OCBBF3000483, OCBBF3008230, PEBLM2004666, PLACE6001185, PUAEN2005930, PUAEN2006701, PUAEN2007044, PUAEN2009655, SPLEN2010912, SPLEN2012624, SPLEN2028914, SPLEN2031424, SPLEN2031547, SPLEN2034781, SPLEN2036932, SYNOV2014400, SYNOV4002346, SYNOV4002883, SYNOV4007430, SYNOV4008440, TESOP2002451, TESTI2049246, TESTI4000014, TESTI4000209, TESTI4001100, TESTI4006137, TESTI4008797, TESTI4009286, TESTI4010377, TESTI4010851, TESTI4010928, TESTI4011161, TESTI4013817, TESTI4014159, TESTI4014694, TESTI4014818, TESTI4021478, TESTI4022936, TESTI4024420, TESTI4027821, TESTI4037156, THYMU2001090, THYMU2023967, THYMU2025707, THYMU2031341, THYMU2033308, THYMU2035735, THYMU2037226, THYMU2039315, THYMU3001234, THYMU3001379, THYMU3004835, THYMU3007137, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3000014, TRACH3001427, TRACH3002192, TRACH3004721, TRACH3005294, TRACH3007479, TRACH3008093, TRACH3009455, TUTER1000122, TUTER2000904, UTERU2004929, UTERU2006115, UTERU2019706, UTERU2021163, UTERU2023039, UTERU2026203, UTERU2030213, UTERU3001572, UTERU3003135, UTERU3005230, UTERU3007640, UTERU3009259, UTERU3009871

ADRGL2000042, BLADE2006830, BRACE2003609, BRAMY3004800, BRAWH1000369, BRAWH2006207, BRAWH2006395, BRAWH2008993, BRAWH2009393, BRAWH2010552, BRAWH3007441, BRAWH3009017, BRHIP2002722, BRHIP2003272, BRHIP2005271, BRHIP2005724, BRHIP2006617, BRHIP2008389, BRHIP2012360, BRHIP2017553, BRHIP2026877, BRHIP3000017, BRHIP3000240, BRHIP3008314, BRHIP3026052, BRTHA2018443, BRTHA3003000, CTONG2020374, CTONG2020378, CTONG2024031, CTONG3004726, FCBBF1000509, FEBRA2001990, FEBRA2006519, FEBRA2028516, HCHON2000743, IMR322001879, NT2RI2009583, OCBBF2006987, OCBBF2008144, OCBBF2030116, PERIC2007068, PUAEN2006335, SPLEN2039379, TESTI2015626, TESTI4000214, TESTI4001984, TESTI4008058, TESTI4013894, TESTI4025268, TESTI4025547, TESTI4026207, TESTI4032090, THYMU3000360, TLIVE2002046, TRACH3000134, UTERU2008040, UTERU2021820, UTERU2028734

These genes are involved in memory and dementia.

Genes Involved in Equilibrium Sense and Movement Function

The cerebellum is the center of equilibrium sense, muscular movement, and motor learning. This area is thought to be involved in motor control, and smooth movements are achieved unconsciously due to cerebellum action. Recent studies have elucidated that the cerebellum participates in not only simple movements but also in establishing higher-order movements such as reading and writing. Thus, genes whose expression levels differ between tissues of the whole brain and the cerebellum are expected to be involved in equilibrium sense or motor function, which can be useful for elucidating the molecular mechanism controlled by the brain. cDNA libraries derived from the cerebellum (BRACE) and from the whole tissues of a normal brain (BRAWH) were analyzed and compared (Table 7). Genes whose expression levels differed between the two were the 488 clones and 66 clones listed below.

ADRGL2009146, ADRGL2012038, ASTRO1000009, ASTRO2003960, BLADE1000176, BLADE2004089, BLADE2008398, BRACE1000186, BRACE1000258, BRACE1000533, BRACE1000572, BRACE2003639, BRACE2005457, BRACE2006319, BRACE2008594, BRACE201.0489, BRACE2011747, BRACE2014306, BRACE2014475, BRACE2014657, BRACE2015058, BRACE2015314, BRACE2016981, BRACE2018762, BRACE2024627, BRACE2026836, BRACE2027258, BRACE2027970, BRACE2028970, BRACE2029112, BRACE2029849, BRACE2030326, BRACE2030341, BRACE2030884, BRACE2031154, BRACE2031389, BRACE2031527, BRACE2031531, BRACE2031899, BRACE2032044, BRACE2032329, BRACE2032385, BRACE2032538, BRACE2032823, BRACE2033720, BRACE2035381, BRACE2035441, BRACE2036005, BRACE2036096, BRACE2036830, BRACE2036834, BRACE2037847, BRACE2038114, BRACE2038329, BRACE2038551, BRACE2039249, BRACE2039327, BRACE2039475, BRACE2039734, BRACE2040138, BRACE2040325, BRACE2041009, BRACE2041200, BRACE2041264, BRACE2042550, BRACE2043142, BRACE2043248, BRACE2043349, BRACE2043665, BRACE2044286, BRACE2044816, BRACE2044949, BRACE2045300, BRACE2045428, BRACE2045596, BRACE2045772, BRACE2045947, BRACE2045954, BRACE2046251, BRACE2046295, BRACE2047011, BRACE2047350, BRACE2047377, BRACE2047385, BRACE3000071, BRACE3000697, BRACE3000787, BRACE3000840, BRACE3000973, BRACE3001002, BRACE3001217, BRACE3001391, BRACE3001595, BRACE3001754, BRACE3002298, BRACE3002390, BRACE3002508, BRACE3003004, BRACE3003192, BRACE3003595, BRACE3003698, BRACE3004058, BRACE3004113, BRACE3004150, BRACE3004358, BRACE3004435, BRACE3004772, BRACE3004783, BRACE3004843, BRACE3004880, BRACE3005145, BRACE3005225, BRACE3005430, BRACE3005499, BRACE3006185, BRACE3006226, BRACE3006462, BRACE3006872, BRACE3007322, BRACE3007472, BRACE3007480, BRACE3007559, BRACE3007625, BRACE3007642, BRACE3007767, BRACE3008036, BRACE3008092, BRACE3008137, BRACE3008384, BRACE3008720, BRACE3008772, BRACE3009090, BRACE3009237, BRACE3009297, BRACE3009377, BRACE3009574, BRACE3009701, BRACE3009708, BRACE3009724, BRACE3009747, BRACE3010397, BRACE3010428, BRACE3011271, BRACE3011421, BRACE3011505, BRACE3012364, BRACE3012930, BRACE3013119, BRACE3013576, BRACE3013740, BRACE3013780, BRACE3014005, BRACE3014068, BRACE3014231, BRACE3014317, BRACE3014807, BRACE3015027, BRACE3015121, BRACE3015262, BRACE3015521, BRACE3015894, BRACE3016884, BRACE3018308, BRACE3018963, BRACE3019055, BRACE3019084, BRACE3020194, BRACE3020286, BRACE3020594, BRACE3022769, BRACE3023912, BRACE3024073, BRACE3024659, BRACE3024662, BRACE3025153, BRACE3025457, BRACE3025531, BRACE3025630, BRACE3026008, BRACE3026075, BRACE3026735, BRACE3027242, BRACE3027326, BRACE3027478, BRACE3030103, BRACE3031838, BRACE3032983, BRACE3040856, BRACE3045033, BRALZ2011796, BRAMY2003008, BRAMY2005052, BRAMY2019300, BRAMY2019963, BRAMY2020058, BRAMY2030098, BRAMY2031317, BRAMY2036567, BRAMY2037823, BRAMY2039872, BRAMY2040592, BRAMY2044078, BRAMY2047420, BRAMY3002620, BRAMY3002803, BRAMY3004224, BRAMY3005091, BRAMY3005932, BRAMY4000229, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2009432, BRCAN2010376, BRCAN2015371, BRCAN2020710, BRCOC2003213, BRCOC2007034, BRCOC2014033, BRCOC2020142, BRHIP2000920, BRHIP2004359, BRHIP2005719, BRHIP2005752, BRHIP2007741, BRHIP2013699, BRHIP2014228, BRHIP2024146, BRHIP3000339, BRHIP3006683, BRHIP3007586, BRHIP3008313, BRHIP3008405, BRHIP3018797, BRSSN2000684, BRSSN2006892, BRSSN2011262, BRSSN2011738, BRSSN2014299, BRSTN2008052, BRSTN2010750, BRSTN2015015, BRSTN2016470, BRTHA1000311, BRTHA2008335, BRTHA2008955, BRTHA2011194, BRTHA3001721, BRTHA3002427, BRTHA3003490, BRTHA3008520, BRTHA3009090, BRTHA3017848, COLON2001721, CTONG2008233, CTONG2017500, CTONG2028124, CTONG3000657, CTONG3001123, CTONG3005813, CTONG3008894, CTONG3009328, DFNES2011499, FCBBF2001183, FCBBF3001977, FEBRA2006396, FEBRA2007544, FEBRA2007708, FEBRA2007801, FEBRA2008287, FEBRA2020886, FEBRA2021966, FEBRA2026984, FEBRA2028618, HCASM2007047, HCHON2000244, HCHON2000626, HCHON2001217, HCHON2002676, HCHON2005921, HCHON2006250, HEART1000074, HHDPC1000118, HLUNG2002465, IMR322000127, IMR322001380, IMR322002035, KIDNE2000665, KIDNE2006580, MESAN2006563, MESAN2012054, MESTC1000042, NB9N41000340, NESOP2001752, NOVAR2001783, NT2NE2006909, NT2RI2005166, NT2RI2008724, NT2RI2012659, NT2RI2014733, NT2RI2019751, NT2RI3002892, NT2RI3003382, NT2RI3004510, NT2RI3005724, NT2RI3006284, NT2RI3006673, NT2RI3007291, NT2RI3007543, NT2RI3008055, NT2RP7004123, NT2RP7005529, NT2RP7009147, NT2RP7010599, NT2RP7014005, NT2RP7017474, NTONG2005969, OCBBF2001794, OCBBF2003819, OCBBF2006005, OCBBF2006151, OCBBF2006764, OCBBF2007028, OCBBF2010140, OCBBF2020343, OCBBF2020741, OCBBF2021286, OCBBF2022351, OCBBF2024850, OCBBF2025527, OCBBF2028935, OCBBF2036743, OCBBF2038317, OCBBF3000483, OCBBF3007516, OCBBF3008230, PEBLM2004666, PERIC2000889, PLACE6001185, PUAEN2002489, PUAEN2005930, PUAEN2006701, PUAEN2007044, PUAEN2009655, SPLEN2010912, SPLEN2012624, SPLEN2027268, SPLEN2028914, SPLEN2031424, SPLEN2031547, SPLEN2034781, SPLEN2036932, SPLEN2037194, SYNOV2014400, SYNOV4002346, SYNOV4002883, SYNOV4007430, SYNOV4007671, SYNOV4008440, TESOP2002273, TESOP2002451, TESOP2002950, TESTI1000330, TESTI4000014, TESTI4000209, TESTI4000349, TESTI4001100, TESTI4001561, TESTI4006137, TESTI4008797, TESTI4009286, TESTI4010851, TESTI4011161, TESTI4013675, TESTI4013817, TESTI4014159, TESTI4014306, TESTI4014694, TESTI4021478, TESTI4022936, TESTI4024420, TESTI4027821, TESTI4037156, TESTI4046819, THYMU2001090, THYMU2016523, THYMU2023967, THYMU2030264, THYMU2033308, THYMU2035735, THYMU2039315, THYMU2039780, THYMU3001083, THYMU3001234, THYMU3003309, THYMU3006485, THYMU3008171, TKIDN2009641, TKIDN2009889, TKIDN2015788, TRACH1000205, TRACH2001549, TRACH2005811, TRACH2006049, TRACH2007834, TRACH2008300, TRACH2025535, TRACH3001427, TRACH3002192, TRACH3004721, TRACH3005294, TRACH3006038, TRACH3006412, TRACH3007479, TRACH3008093, TRACH3009455, TUTER2000904, UTERU2002410, UTERU2006115, UTERU2007520, UTERU2019706, UTERU2023039, UTERU2026203, UTERU3000226, UTERU3001572, UTERU3005230, UTERU3005460, UTERU3005970, UTERU3006308, UTERU3007419, UTERU3007640, UTERU3007913, UTERU3009871

ADRGL2000042, BLADE2006830, BRACE2002589, BRACE2003609, BRACE2009318, BRACE2011677, BRACE2029396, BRACE2037299, BRACE2039823, BRACE2039832, BRACE2043105, BRACE3001058, BRACE3001113, BRACE3003026, BRACE3003053, BRACE3009127, BRACE3010076, BRACE3015829, BRACE3021148, BRAMY3004800, BRAWH1000369, BRAWH2006207, BRAWH2006395, BRAWH2008993, BRAWH2009393, BRAWH2010552, BRAWH3007441, BRAWH3009017, BRCOC2019841, BRHIP2005271, BRHIP3000017, BRHIP3000240, BRTHA2018443, BRTHA3003000, CTONG2020374, CTONG2020378, CTONG2024031, FCBBF1000509, FEBRA2001990, FEBRA2006519, FEBRA2028516, HCHON2000743, IMR322001879, NT2RI2009583, NT2RP8000521, OCBBF2008144, OCBBF2011669, PERIC2007068, PUAEN2006335, SPLEN2039379, SYNOV2021953, TESTI2015626, TESTI4001984, TESTI4008058, TESTI4013894, TESTI4025268, TESTI4032090, THYMU2004284, THYMU2040925, THYMU3000360, TLIVE2002046, TRACH3000134, UTERU2008040, UTERU2011220, UTERU2021820, UTERU2028734

These genes are involved in equilibrium sense or motor function.

Genes Involved in Signaling from Sensory Organs

The thalamus is an area which comprises many neurons strongly connected to the cerebrum, and which transmits sensory information from the spinal cord or such to the responsible area of the cerebrum. The thalamus also controls the direction of movement from the cerebrum. For example, the thalamus resolves vision into the elements of size, shape, and color, and resolves sound into volume and sweetness or harshness to the ear, and then transmits this information to the sensory area of the cerebral cortex. Thus, genes whose expression levels differ between tissues of the whole brain and the thalamus are expected to be involved in signaling from sensory organs. These genes can be used to elucidate the molecular mechanism underlying signaling controlled by the brain. cDNA libraries derived from the thalamus (BRTHA) and from whole tissues of a normal brain (BRAWH) were analyzed and compared (Table 8). Genes whose expression levels differed between the two were the 412 clones and 56 clones listed below.

ASTRO1000009, ASTRO3000482, BLADE2008398, BRACE1000186, BRACE1000258, BRACE1000533, BRACE2005457, BRACE2010489, BRACE2014306, BRACE2014657, BRACE2015058, BRACE2031154, BRACE2035381, BRACE2044286, BRACE2045954, BRACE3000787, BRACE3003192, BRACE3005499, BRACE3007480, BRACE3008384, BRACE3009237, BRACE3009724, BRACE3009747, BRACE3010397, BRACE3010428, BRACE3011271, BRACE3011421, BRACE3012364, BRACE3022769, BRACE3026735, BRACE3027478, BRACE3031838, BRALZ2011796, BRAMY2003008, BRAMY2005052, BRAMY2019300, BRAMY2019963, BRAMY2028914, BRAMY2031317, BRAMY2036567, BRAMY2037823, BRAMY2040592, BRAMY2044078, BRAMY3002803, BRAMY3004224, BRAMY3005091, BRAMY4000229, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2006297, BRCOC2003213, BRCOC2014033, BRCOC2020142, BRHIP2000819, BRHIP2000920, BRHIP2005719, BRHIP2007741, BRHIP2009474, BRHIP2013699, BRHIP2014228, BRHIP2022221, BRHIP2024146, BRHIP3000339, BRHIP3006683, BRHIP3007586, BRHIP3008405, BRHIP3018797, BRSSN2000684, BRSSN2008549, BRSSN2008797, BRSSN2011738, BRSSN2014299, BRSTN2004863, BRSTN2008052, BRSTN2015015, BRSTN2016470, BRTHA1000311, BRTHA2000855, BRTHA2001462, BRTHA2002115, BRTHA2002281, BRTHA2002376, BRTHA2002442, BRTHA2002493, BRTHA2002608, BRTHA2002808, BRTHA2003030, BRTHA2003110, BRTHA2003116, BRTHA2003461, BRTHA2004821, BRTHA2004978, BRTHA2005579, BRTHA2005956, BRTHA2006075, BRTHA2006146, BRTHA2006194, BRTHA2007122, BRTHA2007422, BRTHA2007603, BRTHA2008316, BRTHA2008335, BRTHA2008527, BRTHA2008535, BRTHA2008955, BRTHA2009311, BRTHA2009846, BRTHA2009972, BRTHA2010073, BRTHA2010608, BRTHA2010884, BRTHA2010907, BRTHA2011194, BRTHA2011351, BRTHA2011500, BRTHA2011641, BRTHA2012392, BRTHA2012562, BRTHA2012980, BRTHA2013262, BRTHA2013460, BRTHA2013707, BRTHA2014792, BRTHA2014828, BRTHA2015406, BRTHA2015478, BRTHA2015696, BRTHA2015878, BRTHA2016215, BRTHA2016496, BRTHA2016543, BRTHA2017353, BRTHA2017985, BRTHA2018165, BRTHA2018344, BRTHA2018591, BRTHA2018624, BRTHA2018707, BRTHA2019014, BRTHA2019022, BRTHA2019048, BRTHA3000273, BRTHA3000297, BRTHA3000633, BRTHA3001721, BRTHA3002401, BRTHA3002427, BRTHA3002933, BRTHA3003074, BRTHA3003343, BRTHA3003449, BRTHA3003474, BRTHA3003490, BRTHA3004475, BRTHA3005046, BRTHA3006856, BRTHA3007113, BRTHA3007148, BRTHA3007319, BRTHA3007769, BRTHA3008143, BRTHA3008310, BRTHA3008386, BRTHA3008520, BRTHA3008778, BRTHA3009037, BRTHA3009090, BRTHA3009291, BRTHA3010366, BRTHA3013884, BRTHA3015815, BRTHA3015910, BRTHA3016845, BRTHA3016917, BRTHA3017047, BRTHA3017589, BRTHA3017848, BRTHA3018514, BRTHA3018617, BRTHA3018656, BRTHA3019105, COLON2001721, CTONG1000087, CTONG2008233, CTONG2017500, CTONG2019788, CTONG2023021, CTONG2028124, CTONG3000657, CTONG3001123, CTONG3008894, CTONG3009028, CTONG3009239, CTONG3009328, FCBBF2001183, FCBBF3001977, FCBBF3021576, FEBRA2007544, FEBRA2007801, FEBRA2008287, FEBRA2008360, FEBRA2020886, FEBRA2028618, HCASM2007047, HCHON2000028, HCHON2000212, HCHON2000244, HCHON2000626, HCHON2001084, HCHON2001217, HCHON2002676, HCHON2005921, HCHON2006250, HEART1000074, HEART2007031, HHDPC1000118, HLUNG2001996, HLUNG2002465, IMR322000127, IMR322001380, IMR322002035, KIDNE2002252, KIDNE2005543, KIDNE2006580, KIDNE2011314, MESAN2006563, MESAN2012054, MESTC1000042, NOVAR2001783, NT2NE2006909, NT2RI2008724, NT2RI2012659, NT2RI2014733, NT2RI3002842, NT2RI3002892, NT2RI3005403, NT2RI3006284, NT2RI3006673, NT2RI3007543, NT2RI3008055, NT2RP7004123, NT2RP7005529, NT2RP7009147, NT2RP7014005, NT2RP7017474, NTONG2005969, NTONG2008088, OCBBF2001794, OCBBF2006005, OCBBF2006764, OCBBF2007028, OCBBF2010140, OCBBF2020639, OCBBF2021286, OCBBF2024719, OCBBF2024850, OCBBF2028935, OCBBF2036743, OCBBF2038317, OCBBF3000483, OCBBF3008230, PEBLM2004666, PLACE6001185, PUAEN2002489, PUAEN2005930, PUAEN2006701, PUAEN2007044, PUAEN2009655, RECTM2001347, SKMUS2000757, SPLEN2006122, SPLEN2010912, SPLEN2025491, SPLEN2028914, SPLEN2031424, SPLEN2031547, SPLEN2032154, SPLEN2034781, SPLEN2036821, SPLEN2036932, SYNOV1000374, SYNOV2014400, SYNOV4002346, SYNOV4002883, SYNOV4007430, SYNOV4007671, SYNOV4008440, TESOP2002451, TESTI2049246, TESTI4000014, TESTI4000209, TESTI4001100, TESTI4002290, TESTI4006137, TESTI4008797, TESTI4009286, TESTI4010851, TESTI4012702, TESTI4013817, TESTI4014159, TESTI4014694, TESTI4021478, TESTI4022936, TESTI4024420, TESTI4027821, TESTI4037156, THYMU2001090, THYMU2025707, THYMU2032825, THYMU2033308, THYMU2033787, THYMU2035735, THYMU2039315, THYMU2040975, THYMU3001234, THYMU3001379, THYMU3004835, THYMU3008171, TKIDN2009641, TKIDN2009889, TKIDN2015788, TLIVE2001327, TRACH1000205, TRACH2001549, TRACH2005811, TRACH2006049, TRACH2007834, TRACH2008300, TRACH2023299, TRACH2025535, TRACH3001427, TRACH3002192, TRACH3004068, TRACH3004721, TRACH3005294, TRACH3007479, TRACH3008093, TRACH3009455, TSTOM1000135, TUTER2000904, UTERU2002410, UTERU2006115, UTERU2019706, UTERU2019940, UTERU2023039, UTERU2023175, UTERU2026203, UTERU2030280, UTERU3000899, UTERU3001571, UTERU3001572, UTERU3004709, UTERU3005230, UTERU3005907, UTERU3007640, UTERU3009871

ADRGL2000042, BLADE2006830, BRACE2003609, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRTHA2002133, BRTHA2002702, BRTHA2007060, BRTHA2010033, BRTHA2011321, BRTHA2013426, BRTHA2013610, BRTHA2016318, BRTHA2017364, BRTHA2017972, BRTHA2018011, BRTHA2018443, BRTHA3000296, BRTHA3003000, BRTHA3008826, CTONG2008721, CTONG2020374, CTONG2020378, CTONG2024031, FCBBF1000509, FEBRA2001990, FEBRA2006519, FEBRA2028516, HCHON2000743, HSYRA2005628, IMR322001879, NT2RI2009583, OCBBF2008144, PERIC2007068, PUAEN2006335, SPLEN2016932, SPLEN2039379, SYNOV2006620, TESTI4001984, TESTI4008058, TESTI4025268, TESTI4032090, THYMU3000360, TLIVE2002046, TRACH3000134, UTERU2021820, UTERU2028734

These genes are involved in signaling from sensory organs.

Genes Involved in Emotional Reaction

The amygdala is the center of emotion in the brain. Information passing through the amygdala induces an emotional reaction, for example, panic or fear. When a strong fear reaction is produced due to the emotional evaluation of stimulus in the amygdala, the amygdala transmits an alert signal to each area of the brain. This results in various reactions such as sweating palms, palpitation, elevated blood pressure, and rapid secretion of adrenaline. In other words, the amygdala transmits signals which cause the body to be on the alert and is a tissue involved in a kind of defense instinct. Thus, genes whose expression levels differ between tissues of the whole brain and the amygdala are expected to be involved in emotional reaction. Such genes can be used to elucidate the molecular mechanism underlying emotional reaction, fear, or panic. cDNA libraries derived from the amygdale (BRAMY) and from whole tissues of a normal brain (BRAWH) were analyzed and compared (Table 9). Genes whose expression levels differed between the two were the 383 clones and 55 clones listed below.

ASTRO1000009, BLADE2008398, BRACE1000186, BRACE1000258, BRACE1000533, BRACE2005457, BRACE2006319, BRACE2010489, BRACE2014657, BRACE2015058, BRACE2027258, BRACE2030341, BRACE2031389, BRACE2035381, BRACE2044286, BRACE2045954, BRACE3000787, BRACE3000973, BRACE3003192, BRACE3005499, BRACE3007480, BRACE3008036, BRACE3009237, BRACE3009724, BRACE3009747, BRACE3010428, BRACE3011271, BRACE3011421, BRACE3012364, BRACE3013780, BRACE3022769, BRACE3026735, BRACE3027478, BRACE3031838, BRALZ2011796, BRAMY2001473, BRAMY2003008, BRAMY2004771, BRAMY2005052, BRAMY2017528, BRAMY2019300, BRAMY2019963, BRAMY2019985, BRAMY2020058, BRAMY2020270, BRAMY2021498, BRAMY2028856, BRAMY2028914, BRAMY2029602, BRAMY2030098, BRAMY2030109, BRAMY2030702, BRAMY2030703, BRAMY2030799, BRAMY2031317, BRAMY2031377, BRAMY2031442, BRAMY2032014, BRAMY2032242, BRAMY2032317, BRAMY2033003, BRAMY2033116, BRAMY2033267, BRAMY2033594, BRAMY2034185, BRAMY2034920, BRAMY2034993, BRAMY2036387, BRAMY2036396, BRAMY2036567, BRAMY2036699, BRAMY2036913, BRAMY2037823, BRAMY2038100, BRAMY2038484, BRAMY2038846, BRAMY2038904, BRAMY2039872, BRAMY2040478, BRAMY2040592, BRAMY2041261, BRAMY2041378, BRAMY2041542, BRAMY2042612, BRAMY2042641, BRAMY2042760, BRAMY2042918, BRAMY2044078, BRAMY2044246, BRAMY2045036, BRAMY2046478, BRAMY2046742, BRAMY2046989, BRAMY2047169, BRAMY2047420, BRAMY2047676, BRAMY2047746, BRAMY2047751, BRAMY2047765, BRAMY2047884, BRAMY3000206, BRAMY3000213, BRAMY3001401, BRAMY3001794, BRAMY3002312, BRAMY3002620, BRAMY3002803, BRAMY3002805, BRAMY3004224, BRAMY3004672, BRAMY3004900, BRAMY3004919, BRAMY3005091, BRAMY3005932, BRAMY3006297, BRAMY3007206, BRAMY3007609, BRAMY3008466, BRAMY3008505, BRAMY3008650, BRAMY3009811, BRAMY3010411, BRAMY4000095, BRAMY4000229, BRAMY4000277, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2014881, BRCAN2017717, BRCOC2000670, BRCOC2003213, BRCOC2014033, BRCOC2020142, BRHIP2000920, BRHIP2005719, BRHIP2007741, BRHIP2014228, BRHIP2024146, BRHIP2026061, BRHIP3000339, BRHIP3001283, BRHIP3006683, BRHIP3007586, BRHIP3008405, BRHIP3018797, BRSSN2000684, BRSSN2004496, BRSSN2011738, BRSSN2014299, BRSTN2008052, BRSTN2010750, BRSTN2015015, BRSTN2016470, BRTHA1000311, BRTHA2008335, BRTHA2011641, BRTHA3001721, BRTHA3002427, BRTHA3003490, BRTHA3004475, BRTHA3008520, BRTHA3009090, BRTHA3017848, COLON2001721, CTONG1000087, CTONG2008233, CTONG2017500, CTONG2028124, CTONG3000657, CTONG3001123, CTONG3008894, CTONG3009239, CTONG3009328, FCBBF2001183, FCBBF3001977, FEBRA2007544, FEBRA2007801, FEBRA2008287, FEBRA2010719, FEBRA2020886, FEBRA2025427, FEBRA2028618, HCASM2007047, HCHON2000244, HCHON2000626, HCHON2001217, HCHON2002676, HCHON2006250, HCHON2008112, HEART1000074, HHDPC1000118, HLUNG2002465, HSYRA2009075, IMR322000127, IMR322001380, IMR322002035, KIDNE2000665, KIDNE2006580, MESAN2006563, MESAN2012054, MESAN2015515, MESTC1000042, NOVAR2001783, NT2NE2005890, NT2NE2006909, NT2RI2008724, NT2RI2012659, NT2RI2014733, NT2RI3001515, NT2RI3002892, NT2RI3005724, NT2RI3006284, NT2RI3006673, NT2RI3007543, NT2RI3008055, NT2RP7005529, NT2RP7009147, NT2RP7014005, NT2RP7017474, NTONG2005969, OCBBF1000254, OCBBF2001794, OCBBF2006005, OCBBF2006764, OCBBF2007028, OCBBF2007114, OCBBF2010140, OCBBF2021286, OCBBF2023162, OCBBF2024850, OCBBF2028935, OCBBF2035214, OCBBF2036743, OCBBF2038317, OCBBF3000483, OCBBF3008230, PEBLM2004666, PERIC2000889, PERIC2003720, PLACE6001185, PUAEN2005930, PUAEN2006701, PUAEN2007044, PUAEN2009174, PUAEN2009655, SKNMC2002402, SKNSH2000482, SPLEN2001599, SPLEN2002467, SPLEN2028914, SPLEN2029912, SPLEN2031424, SPLEN2031547, SPLEN2034781, SPLEN2036932, SPLEN2038345, SYNOV2014400, SYNOV4002346, SYNOV4002883, SYNOV4007430, SYNOV4007671, SYNOV4008440, TESOP2002451, TESTI2009474, TESTI4000014, TESTI4000209, TESTI4001100, TESTI4006137, TESTI4008797, TESTI4009286, TESTI4010851, TESTI4013817, TESTI4014159, TESTI4014694, TESTI4021478, TESTI4022936, TESTI4024420, TESTI4027821, TESTI4029836, TESTI4037156, TESTI4037188, THYMU2001090, THYMU2014353, THYMU2033308, THYMU2035735, THYMU2037226, THYMU2039315, THYMU3001234, THYMU3001379, THYMU3004835, THYMU3008171, TKIDN2009641, TKIDN2009889, TKIDN2015788, TLIVE2004320, TRACH1000205, TRACH2001549, TRACH2001684, TRACH2005811, TRACH2006049, TRACH2007834, TRACH2008300, TRACH2025344, TRACH2025535, TRACH2025911, TRACH3001427, TRACH3002192, TRACH3004068, TRACH3004721, TRACH3005294, TRACH3007479, TRACH3008093, TRACH3009455, TUTER2000904, UTERU2002410, UTERU2004929, UTERU2006115, UTERU2007520, UTERU2019706, UTERU2023039, UTERU2026203, UTERU3001572, UTERU3001766, UTERU3005230, UTERU3007640, UTERU3009517, UTERU3009871

ADRGL2000042, BLADE2006830, BRACE2003609, BRACE2039823, BRAMY2019111, BRAMY2035070, BRAMY2035449, BRAMY2035718, BRAMY2038516, BRAMY2039341, BRAMY2040159, BRAMY2041434, BRAMY2045471, BRAMY3004800, BRAWH1000369, BRAWH2006207, BRAWH2006395, BRAWH2008993, BRAWH2009393, BRAWH2010552, BRAWH3007441, BRAWH3009017, BRHIP2005271, BRHIP3000017, BRTHA2018443, BRTHA3003000, CTONG2020374, CTONG2020378, CTONG2024031, FCBBF1000509, FEBRA2001990, FEBRA2006519, FEBRA2028516, HCHON2000508, HCHON2000743, IMR322001879, NT2RI2009583, OCBBF2008144, PERIC2007068, PUAEN2006335, SPLEN2039379, TESTI2015626, TESTI2026647, TESTI4001984, TESTI4008058, TESTI4013894, TESTI4025268, TESTI4032090, THYMU3000360, TKIDN2018926, TLIVE2002046, TRACH3000134, UTERU2008040, UTERU2021820, UTERU2028734

These genes are involved in emotional reaction.

Cancer-Related Genes

Cancer tissues are assumed to express a distinct set of genes distinct from normal tissues, and thus expression of these genes can contribute to carcinogenesis in tissues and cells. Thus, genes whose expression patterns in cancer tissues differ from those in normal tissues are cancer-related genes. A search was carried out for genes whose expression levels in cancer tissues differed from those in normal tissues.

The result of comparative analysis of cDNA libraries derived from breast tumor (TBAES) and normal breast (BEAST) (Table 10) showed that the genes whose expression levels differed between the two were 35 and four clones as described below.

ASTRO2002842, BRACE3016884, BRSSN2011262, BRTHA2008335, HCHON2000244, HCHON2006250, HEART1000010, MESAN2012054, NT2RP7000466, NT2RP7009147, OCBBF2021020, PEBLM2002749, PEBLM2004666, SPLEN2001599, SPLEN2031547, STOMA1000189, TBAES2001171, TBAES2001220, TBAES2001229, TBAES2001258, TBAES2001492, TBAES2001751, TBAES2002197, TBAES2003550, TBAES2004055, TBAES2005157, TBAES2005543, TBAES2006568, TBAES2007964, TESTI4000014, TESTI4037156, TRACH3002192, TRACH3004068, TSTOM2000553, UTERU2002410

BRAWH2006395, NT2RI2009583, STOMA2004893, TBAES2000932

The result of comparative analysis of cDNA libraries derived from cervical tumor (TCERX) and normal cervical duct (CERVX) (Table 11) showed that the genes whose expression levels differed between the two were twelve and two clones as described below.

BLADE2007666, BRAMY2047420, BRCAN2007409, BRSTN2016470, CERVX1000042, CERVX2002006, MESAN2006563, PROST2018090, TCERX2000613, TESTI4037156, THYMU2031341, UTERU2004688 CERVX2002013, NT2RI2009583

The result of comparative analysis of cDNA libraries derived from colon tumor (TCOLN) and normal colon (COLON) (Table 12) showed that the genes whose expression levels were different between the two were 24 and four clones as described below.

BRACE3015027, BRAMY2040592, BRSTN2016470, COLON1000030, COLON2000470, COLON2000568, COLON2001721, COLON2002443, COLON2002520, COLON2003043, COLON2004478, COLON2005126, COLON2005772, COLON2006282, COLON2009499, OCBBF2028935, PLACE7000514, RECTM2000433, SYNOV4007671, TCOLN2002278, TESTI2052693, TESTI4037156, THYMU2031368, TRACH2025535 CTONG1000113, NT2RI2009583, NT2RI2018448, TESTI2015626

The result of comparative analysis of cDNA libraries derived from esophageal tumor (TESOP) and normal esophagus (NESOP) (Table 13) showed that the genes whose expression levels were different between the two were 56 and ten clones as described below.

BRACE2030341, BRAMY2047420, BRHIP2003917, BRTHA2003461, CTONG2013178, D3OST3000169, FEBRA2025427, HCHON2000244, HHDPC1000118, NESOP2000744, NESOP2001433, NESOP2001656, NESOP2001694, NESOP2001752, NESOP2002738, NT2RI3006284, NT2RP7009147, PLACE6019932, SYNOV2005216, TESOP1000127, TESOP2000801, TESOP2001122, TESOP2001166, TESOP2001345, TESOP2001605, TESOP2001818, TESOP2001849, TESOP2001865, TESOP2001953, TESOP2002273, TESOP2002451, TESOP2002489, TESOP2002539, TESOP2002950, TESOP2003273, TESOP2003753, TESOP2004114, TESOP2005285, TESOP2005485, TESOP2005579, TESOP2006041, TESOP2006060, TESOP2006068, TESOP2006670, TESOP2006746, TESOP2007052, TESOP2007262, TESOP2007636, TESOP2007688, TESOP2009121, TESOP2009555, TESTI4009286, TESTI4010851, THYMU2040975, TRACH2005811, UTERU2023175 CTONG2016942, NT2RI2009583, TESOP2000390, TESOP2001796, TESOP2005199, TESOP2006398, TESOP2006865, TESOP2007384, TESTI2015626, TRACH2000862

The result of comparative analysis of cDNA libraries derived from kidney tumor (TKIDN) and normal kidney (KIDNE) (Table 14) showed that the genes whose expression levels were different between the two were 96 and 13 clones as described below.

ASTRO2018373, BRACE1000186, BRACE2014306, BRACE2015058, BRACE2016981, BRACE2043665, BRACE3008036, BRACE3010428, BRACE3022769, BRAMY2019963, BRAMY2044078, BRAWH1000127, BRAWH2001395, BRAWH2001671, BRAWH2013294, BRAWH2014645, BRHIP2024146, BRHIP3000339, BRSSN2000684, BRSSN2004719, BRSSN2018581, BRSTN2016470, BRTHA1000311, BRTHA3002427, CTONG1000087, CTONG2028124, CTONG3000657, CTONG3008894, FCBBF2001183, FEBRA2008287, HCASM2001301, HCHON2000028, HCHON2000244, HEART1000074, HHDPC1000118, HSYRA2008376, KIDNE1000064, KIDNE2000665, KIDNE2000722, KIDNE2000832, KIDNE2000846, KIDNE2001361, KIDNE2001847, KIDNE2002252, KIDNE2002991, KIDNE2003837, KIDNE2005543, KIDNE2006580, KIDNE2010264, KIDNE2011314, KIDNE2011532, KIDNE2011635, KIDNE2012945, KIDNE2013095, NESOP2001656, NTONG2005969, PEBLM2004666, SKMUS2000757, STOMA1000189, SYNOV4007671, TBAES2001258, TESTI4000014, TESTI4001100, TESTI4012702, TESTI4046819, THYMU2032014, TKIDN2000701, TKIDN2002424, TKIDN2002632, TKIDN2003044, TKIDN2004386, TKIDN2005934, TKIDN2005947, TKIDN2006525, TKIDN2006852, TKIDN2007667, TKIDN2009092, TKIDN2009641, TKIDN2009889, TKIDN2010934, TKIDN2012824, TKIDN2013287, TKIDN2014757, TKIDN2014771, TKIDN2015263, TKIDN2015788, TKIDN2016309, TKIDN2019116, TRACH2001443, TRACH2001684, TRACH2007834, TRACH2008300, TRACH3001427, UTERU2002410, UTERU2023175, UTERU3001572

BLADE2006830, BRALZ2017844, CTONG2028758, FCBBF1000509, FEBRA2001990, FEBRA2028516, HCHON2000508, MESAN2005303, NT2RI2009583, TESTI2015626, TKIDN2008778, TKIDN2012771, TKIDN2018926

The result of comparative analysis of cDNA libraries derived from liver tumor (TLIVE) and normal liver (LIVER) (Table 15) showed that the genes whose expression levels were different between the two were 35 and six clones as described below.

BRCAN2018935, BRSTN2016470, BRTHA2012980, BRTHA3002427, CTONG2028124, LIVER2007415, NT2RI2008724, SPLEN2012624, SPLEN2033098, TESOP2002451, TLIVE2000023, TLIVE2001327, TLIVE2001828, TLIVE2001927, TLIVE2002336, TLIVE2002338, TLIVE2002690, TLIVE2003197, TLIVE2003225, TLIVE2003381, TLIVE2003970, TLIVE2004110, TLIVE2004320, TLIVE2004601, TLIVE2005180, TLIVE2006236, TLIVE2006529, TLIVE2007132, TLIVE2007528, TLIVE2007816, TLIVE2008083, TLIVE2008229, TLIVE2009541, UTERU2002410, UTERU2005621

LIVER2000247, NT2RI2009583, TESTI2015626, TLIVE2001684, TLIVE2002046, TLIVE2007607

The result of comparative analysis of cDNA libraries derived from lung tumor (TLUNG) and normal lung (HLUNG) (Table 16) showed that the genes whose expression levels were different between the two were 47 and nine clones as described below.

BRCAN2021028, BRHIP2000819, BRSTN2016470, CTONG1000087, CTONG2028124, HCHON2006250, HEART1000074, HLUNG1000017, HLUNG2000014, HLUNG2001996, HLUNG2002465, HLUNG2002958, HLUNG2003003, HLUNG2003872, HLUNG2010464, HLUNG2011041, HLUNG2011298, HLUNG2012049, HLUNG2012287, HLUNG2012727, HLUNG2013204, HLUNG2013304, HLUNG2013622, HLUNG2013851, HLUNG2014262, HLUNG2014288, HLUNG2014449, HLUNG2015617, HLUNG2017350, HLUNG2017546, HLUNG2017806, HLUNG2019058, HSYRA2008376, KIDNE2012945, NT2RI2003993, NT2RP7013795, OCBBF3000483, SPLEN2028914, SPLEN2031547, SYNOV4007671, TESOP1000127, TESTI2003573, TESTI4000014, TESTI4037156, TRACH2005811, TRACH3004068, UTERU2005621

FEBRA2028516, HCHON2000508, HLUNG2013350, HLUNG2015418, HLUNG2015548, HLUNG2016862, NT2RI2009583, TESTI2015626, TRACH2019672

The result of comparative analysis of cDNA libraries derived from ovary tumor (TOVER) and normal ovary (NOVER) (Table 17A) showed the genes whose expression levels were different between the two were 16 clones as described below.

CTONG2019788, FEBRA2014213, HLUNG2017546, NOVAR2000136, NOVAR2000710, NOVAR2000962, NOVAR2001108, NOVAR2001783, OCBBF3007516, TESTI2052693, TOVAR2000649, TOVAR2001281, TOVAR2001730, TOVAR2002247, TOVAR2002549, TRACH3004068

The result of comparative analysis of cDNA libraries derived from ovary tumor (TOVER) and normal ovary (NOVER) (Table 17B) showed the gene whose expression levels were different between the two was one clone as described below. The gene has no different expression levels between normal and diseased ovary. However, the gene showed significantly different expression level in both ovary tumor and normal ovary, compared with other tissues. Thus, the gene are ovary-specific gene and can be used as diagnostic marker because its association with the disease.

TESTI2015626

The result of comparative analysis of cDNA libraries derived from stomach tumor (TSTOM) and normal stomach (STOMA) (Table 18) showed that the genes whose expression levels were different between the two were 31 and five clones as described below.

BRACE2024627, BRAWH2014645, BRCAN2028355, BRHIP2000819, BRSTN2016470, BRTHA3003490, COLON2002443, HEART1000010, HLUNG2002465, KIDNE2001847, NT2RP7000466, PUAEN2006328, SMINT2001818, STOMA1000189, STOMA2003444, STOMA2004294, STOMA2004925, STOMA2008546, SYNOV4007671, TESTI4000014, TESTI4010851, THYMU2035735, TRACH2001549, TRACH2005811, TRACH2025535, TSTOM1000135, TSTOM2000442, TSTOM2000553, TSTOM2002672, UTERU2006115, UTERU3001572

The following five clones also had different expression levels between the two.

FEBRA2008692, NT2RI2009583, STOMA2003158, STOMA2004893, TESTI2015626

The result of comparative analysis of cDNA libraries derived from uterine tumor (TUTER) and normal uterus (UTERU) (Table 19) showed that the genes whose expression levels were different between the two were 244 and 34 clones as described below.

BNGH42007788, BRACE1000186, BRACE2030341, BRACE3008772, BRACE3009747, BRACE3010428, BRACE3027478, BRALZ2017359, BRAWH2014645, BRAWH3000314, BRAWH3001326, BRAWH3002574, BRAWH3002821, BRAWH3003727, BRAWH3007592, BRCAN2009432, BRCAN2028355, BRHIP3007586, BRHIP3008344, BRHIP3008565, BRSSN2006892, BRSTN2001067, BRSTN2016470, BRTHA2010608, BRTHA3003074, CTONG1000087, CTONG1000467, CTONG2028124, CTONG3001123, CTONG3008894, CTONG3009028, CTONG3009239, FCBBF3004847, FEBRA2026984, FEBRA2028618, HCHON2000244, HCHON2000418, HCHON2000626, HCHON2001084, HCHON2001217, HCHON2005921, HCHON2006250, HCHON2008444, HLUNG2003003, HSYRA2008376, KIDNE2002252, MESAN2014295, NOVAR2000710, NT2RI2008724, NT2RI2014247, NT2RI2014733, NT2RI3002892, NT2RI3005724, NT2RI3006284, NT2RI3006340, NT2RI3006673, NT2RI3007291, NT2RI3007543, NT2RP7004123, NT2RP7005529, NT2RP7009147, NT2RP7017474, OCBBF2007028, OCBBF2020741, OCBBF2024850, OCBBF2036743, OCBBF3000483, PLACE6001185, PLACE7000514, PUAEN2007044, PUAEN2009655, SKNSH2000482, SPLEN2006122, SPLEN2016554, SPLEN2031547, SPLEN2036932, STOMA1000189, STOMA2004925, SYNOV2017055, SYNOV4001395, SYNOV4002346, SYNOV4008440, TCERX2000613, TESOP2002273, TESTI4000014, TESTI4008797, TESTI4009286, TESTI4012702, TESTI4013675, TESTI4014159, TESTI4018886, TESTI4029671, TESTI4037156, THYMU2008725, THYMU2031890, THYMU2033070, THYMU2035735, THYMU3001472, TRACH1000205, TRACH2001443, TRACH2001549, TRACH2005811, TRACH2007834, TRACH2008300, TRACH3002192, TRACH3003379, TRACH3004068, TRACH3004721, TRACH3007479, TUTER1000122, TUTER2000425, TUTER2000904, TUTER2000916, TUTER2001387, TUTER2002729, UTERU1000024, UTERU1000031, UTERU1000148, UTERU1000249, UTERU1000337, UTERU1000339, UTERU2000649, UTERU2001409, UTERU2002410, UTERU2002841, UTERU2004688, UTERU2004929, UTERU2005004, UTERU2005621, UTERU2006115, UTERU2006137, UTERU2006568, UTERU2007444, UTERU2007520, UTERU2007724, UTERU2008347, UTERU2014678, UTERU2017762, UTERU2019491, UTERU2019681, UTERU2019706, UTERU2019940, UTERU2020491, UTERU2020718, UTERU2021163, UTERU2021380, UTERU2022020, UTERU2022981, UTERU2023039, UTERU2023175, UTERU2023651, UTERU2023712, UTERU2024002, UTERU2024656, UTERU2025025, UTERU2025645, UTERU2025891, UTERU2026025, UTERU2026090, UTERU2026203, UTERU2027591, UTERU2029953, UTERU2030213, UTERU2030280, UTERU2031084, UTERU2031268, UTERU2031521, UTERU2031703, UTERU2031851, UTERU2033375, UTERU2033382, UTERU2035114, UTERU2035323, UTERU2035328, UTERU2035331, UTERU2035452, UTERU2035469, UTERU2035503, UTERU2035745, UTERU2036089, UTERU2037361, UTERU2037577, UTERU2038251, UTERU3000226, UTERU3000645, UTERU3000665, UTERU3000828, UTERU3000899, UTERU3001059, UTERU3001240, UTERU3001542, UTERU3001571, UTERU3001572, UTERU3001585, UTERU3001652, UTERU3001766, UTERU3001988, UTERU3002209, UTERU3002218, UTERU3002383, UTERU3002667, UTERU3002731, UTERU3002768, UTERU3002786, UTERU3002993, UTERU3003116, UTERU3003135, UTERU3003178, UTERU3003465, UTERU3003523, UTERU3003776, UTERU3004523, UTERU3004616, UTERU3004709, UTERU3004992, UTERU3005049, UTERU3005205, UTERU3005230, UTERU3005460, UTERU3005585, UTERU3005907, UTERU3005970, UTERU3006008, UTERU3006308, UTERU3007134, UTERU3007419, UTERU3007640, UTERU3007913, UTERU3008660, UTERU3008671, UTERU3009259, UTERU3009490, UTERU3009517, UTERU3009690, UTERU3009871, UTERU3009979, UTERU3011063, UTERU3015086, UTERU3015500, UTERU3016789, UTERU3018081, UTERU3018154, UTERU3018616, UTERU3018711

ADRGL2000042, BRHIP3000017, CTONG2003348, CTONG2019822, CTONG2020378, CTONG2020411, CTONG2024031, FEBRA2028516, HCASM2008536, HCHON2000743, IMR322001879, MESAN2005303, NT2RI2009583, OCBBF2008144, PERIC2007068, SPLEN2039379, TESTI2015626, TESTI4013894, TUTER2000057, UTERU2004299, UTERU2008040, UTERU2011220, UTERU2019534, UTERU2021820, UTERU2028734, UTERU2032279, UTERU2033577, UTERU2035978, UTERU3000402, UTERU3000738, UTERU3001053, UTERU3014791, UTERU3015412, UTERU3017176

The result of comparative analysis of cDNA libraries derived from tongue cancer (CTONG) and normal tongue (NTONG) (Table 20) showed that the genes whose expression levels were different between the two were 166 and 31 clones as described below.

BNGH42007788, BRACE1000186, BRACE2006319, BRACE3010428, BRACE3012364, BRAMY2020058, BRAMY3002803, BRAWH2001671, BRAWH2014645, BRAWH3002574, BRCAN2009432, BRCAN2015371, BRCAN2020710, BRHIP2004814, BRHIP3018797, BRTHA2003461, BRTHA3003490, CTONG1000087, CTONG1000088, CTONG1000288, CTONG1000302, CTONG1000341, CTONG1000467, CTONG1000488, CTONG1000508, CTONG1000540, CTONG2000042, CTONG2001877, CTONG2004062, CTONG2006798, CTONG2008233, CTONG2009423, CTONG2009531, CTONG2010803, CTONG2013178, CTONG2017500, CTONG2019248, CTONG2019652, CTONG2019704, CTONG2019788, CTONG2019833, CTONG2020127, CTONG2020522, CTONG2020638, CTONG2020806, CTONG2021132, CTONG2022153, CTONG2022601, CTONG2023021, CTONG2023512, CTONG2024206, CTONG2024749, CTONG2025496, CTONG2025516, CTONG2025900, CTONG2026920, CTONG2027327, CTONG2028124, CTONG2028687, CTONG3000084, CTONG3000657, CTONG3000686, CTONG3000707, CTONG3000896, CTONG3001123, CTONG3001370, CTONG3001420, CTONG3001560, CTONG3002020, CTONG3002127, CTONG3002412, CTONG3002674, CTONG3003179, CTONG3003483, CTONG3003652, CTONG3003654, CTONG3003737, CTONG3003905, CTONG3003972, CTONG3004072, CTONG3004712, CTONG3005325, CTONG3005648, CTONG3005713, CTONG3005813, CTONG3006067, CTONG3006186, CTONG3006650, CTONG3007444, CTONG3007528, CTONG3007586, CTONG3007870, CTONG3008252, CTONG3008258, CTONG3008496, CTONG3008566, CTONG3008639, CTONG3008831, CTONG3008894, CTONG3008951, CTONG3009028, CTONG3009227, CTONG3009239, CTONG3009328, CTONG3009385, FEBRA2007544, FEBRA2007801, FEBRA2021966, FEBRA2025427, HCHON2000028, HCHON2001217, HHDPC1000118, HSYRA2008376, KIDNE2001847, KIDNE2002252, MESAN2006563, NT2RI2008724, NT2RI2018883, NT2RI3000622, NT2RI3006284, NT2RI3006673, NT2RI3007543, NT2RI3007757, NT2RP7004123, NT2RP7009147, NT2RP7014005, NTONG2000413, NTONG2003852, NTONG2005277, NTONG2005969, NTONG2006354, NTONG2007249, NTONG2007517, NTONG2008088, NTONG2008672, OCBBF2001794, OCBBF2006151, PEBLM2004666, PEBLM2005183, SPLEN2002467, SPLEN2029912, SPLEN2031547, SYNOV4007671, SYNOV4008440, TBAES2002197, TESOP2002273, TESTI2009474, TESTI4000014, TESTI4000209, TESTI4008018, TESTI4009286, TESTI4010851, TESTI4012702, TESTI4013675, THYMU2031847, THYMU2033308, TLIVE2002690, TRACH2005811, TRACH2007059, TRACH2025535, TRACH3001427, TSTOM2000553, UTERU2005621, UTERU2017762, UTERU2023175, UTERU3001572

BLADE2006830, BRHIP3000017, CTONG1000113, CTONG2003348, CTONG2004000, CTONG2008721, CTONG2015596, CTONG2015633, CTONG2016942, CTONG2019822, CTONG2020374, CTONG2020378, CTONG2020411, CTONG2020974, CTONG2024031, CTONG2028758, CTONG3001501, CTONG3002552, CTONG3003598, CTONG3004550, CTONG3004726, CTONG3009287, FEBRA2008692, FEBRA2028516, HCHON2000508, NT2RI2009583, NTONG2008093, PERIC2007068, TESOP2007384, TLIVE2002046, TRACH2000862

These genes are involved in cancer.

Further, there is a method to search for genes involved in development and differentiation, which is the expression frequency analysis in which the expression levels of genes are compared between developing and/or differentiating tissues and/or cells and adult tissues and/or cells. The genes involved in tissue development and/or differentiation are genes participating in tissue construction and expression of function, and thus are useful genes, which are available for regenerative medicine aiming at convenient regeneration of injured tissues.

By using the information of gene expression frequency gained from the database of nucleotide sequences of 1,402,069 clones as described above, genes whose expression frequencies were different between developing and/or differentiating tissues and/or cells and adult tissues and/or cells were analyzed.

The result of comparative analysis of cDNA libraries derived from fetal brain (FCBBF, FEBRA or OCBBF) and adult brain (BRACE, BRALZ, BRAMY, BRAWH, BRCAN, BRCOC, BRHIP, BRSSN, BRSTN or BRTHA) (Table 21) showed that the genes whose expression levels were different between the two were 1,035 and 139 clones as described below.

ADRGL2009146, ADRGL2012038, ADRGL2012179, ASTRO1000009, ASTRO2003960, ASTRO3000482, BLADE1000176, BLADE2001371, BLADE2004089, BLADE2008398, BNGH42007788, BRACE1000186, BRACE1000258, BRACE1000533, BRACE1000572, BRACE2003639, BRACE2005457, BRACE2006319, BRACE2008594, BRACE2010489, BRACE2011747, BRACE2014306, BRACE2014475, BRACE2014657, BRACE2015058, BRACE2015314, BRACE2016981, BRACE2018762, BRACE2024627, BRACE2026836, BRACE2027258, BRACE2027970, BRACE2028970, BRACE2029112, BRACE2029849, BRACE2030326, BRACE2030341, BRACE2030884, BRACE2031154, BRACE2031389, BRACE2031527, BRACE2031531, BRACE2031899, BRACE2032044, BRACE2032329, BRACE2032385, BRACE2032538, BRACE2032823, BRACE2033720, BRACE2035381, BRACE2035441, BRACE2036005, BRACE2036096, BRACE2036830, BRACE2036834, BRACE2037847, BRACE2038114, BRACE2038329, BRACE2038551, BRACE2039249, BRACE2039327, BRACE2039475, BRACE2039734, BRACE2040138, BRACE2040325, BRACE2041009, BRACE2041200, BRACE2041264, BRACE2042550, BRACE2043142, BRACE2043248, BRACE2043349, BRACE2043665, BRACE2044286, BRACE2044816, BRACE2044949, BRACE2045300, BRACE2045428, BRACE2045596, BRACE2045772, BRACE2045947, BRACE2045954, BRACE2046251, BRACE2046295, BRACE2047011, BRACE2047350, BRACE2047377, BRACE2047385, BRACE3000071, BRACE3000697, BRACE3000787, BRACE3000840, BRACE3000973, BRACE3001002, BRACE3001217, BRACE3001391, BRACE3001595, BRACE3001754, BRACE3002298, BRACE3002390, BRACE3002508, BRACE3003004, BRACE3003192, BRACE3003595, BRACE3003698, BRACE3004058, BRACE3004113, BRACE3004150, BRACE3004358, BRACE3004435, BRACE3004772, BRACE3004783, BRACE3004843, BRACE3004880, BRACE3005145, BRACE3005225, BRACE3005430, BRACE3005499, BRACE3006185, BRACE3006226, BRACE3006462, BRACE3006872, BRACE3007322, BRACE3007472, BRACE3007480, BRACE3007559, BRACE3007625, BRACE3007642, BRACE3007767, BRACE3008036, BRACE3008092, BRACE3008137, BRACE3008384, BRACE3008720, BRACE3008772, BRACE3009090, BRACE3009237, BRACE3009297, BRACE3009377, BRACE3009574, BRACE3009701, BRACE3009708, BRACE3009724, BRACE3009747, BRACE3010397, BRACE3010428, BRACE3011271, BRACE3011421, BRACE3011505, BRACE3012364, BRACE3012930, BRACE3013119, BRACE3013576, BRACE3013740, BRACE3013780, BRACE3014005, BRACE3014068, BRACE3014231, BRACE3014317, BRACE3014807, BRACE3015027, BRACE3015121, BRACE3015262, BRACE3015521, BRACE3015894, BRACE3016884, BRACE3018308, BRACE3018963, BRACE3019055, BRACE3019084, BRACE3020194, BRACE3020286, BRACE3020594, BRACE3022769, BRACE3023912, BRACE3024073, BRACE3024659, BRACE3024662, BRACE3025153, BRACE3025457, BRACE3025531, BRACE3025630, BRACE3026008, BRACE3026075, BRACE3026735, BRACE3027242, BRACE3027326, BRACE3027478, BRACE3030103, BRACE3031838, BRACE3032983, BRACE3040856, BRACE3045033, BRALZ2011796, BRALZ2012183, BRALZ2012848, BRALZ2014484, BRALZ2016085, BRALZ2016498, BRALZ2017359, BRAMY2001473, BRAMY2003008, BRAMY2004771, BRAMY2005052, BRAMY2017528, BRAMY2019300, BRAMY2019963, BRAMY2019985, BRAMY2020058, BRAMY2020270, BRAMY2021498, BRAMY2028856, BRAMY2028914, BRAMY2029602, BRAMY2030098, BRAMY2030109, BRAMY2030702, BRAMY2030703, BRAMY2030799, BRAMY2031317, BRAMY2031377, BRAMY2031442, BRAMY2032014, BRAMY2032242, BRAMY2032317, BRAMY2033003, BRAMY2033116, BRAMY2033267, BRAMY2033594, BRAMY2034185, BRAMY2034920, BRAMY2034993, BRAMY2036387, BRAMY2036396, BRAMY2036567, BRAMY2036699, BRAMY2036913, BRAMY2037823, BRAMY2038100, BRAMY2038484, BRAMY2038846, BRAMY2038904, BRAMY2039872, BRAMY2040478, BRAMY2040592, BRAMY2041261, BRAMY2041378, BRAMY2041542, BRAMY2042612, BRAMY2042641, BRAMY2042760, BRAMY2042918, BRAMY2044078, BRAMY2044246, BRAMY2045036, BRAMY2046478, BRAMY2046742, BRAMY2046989, BRAMY2047169, BRAMY2047420, BRAMY2047676, BRAMY2047746, BRAMY2047751, BRAMY2047765, BRAMY2047884, BRAMY3000206, BRAMY3000213, BRAMY3001401, BRAMY3001794, BRAMY3002312, BRAMY3002620, BRAMY3002803, BRAMY3002805, BRAMY3004224, BRAMY3004672, BRAMY3004900, BRAMY3004919, BRAMY3005091, BRAMY3005932, BRAMY3006297, BRAMY3007206, BRAMY3007609, BRAMY3008466, BRAMY3008505, BRAMY3008650, BRAMY3009811, BRAMY3010411, BRAMY4000095, BRAMY4000229, BRAMY4000277, BRASW1000125, BRAWH1000127, BRAWH2001395, BRAWH2001671, BRAWH2001940, BRAWH2001973, BRAWH2002560, BRAWH2002761, BRAWH2005315, BRAWH2007658, BRAWH2010000, BRAWH2010084, BRAWH2010536, BRAWH2012162, BRAWH2012326, BRAWH2013294, BRAWH2013871, BRAWH2014414, BRAWH2014645, BRAWH2014662, BRAWH2014876, BRAWH2014954, BRAWH2016221, BRAWH2016439, BRAWH2016702, BRAWH2016724, BRAWH3000078, BRAWH3000100, BRAWH3000314, BRAWH3000491, BRAWH3001326, BRAWH3001475, BRAWH3001891, BRAWH3002574, BRAWH3002600, BRAWH3002819, BRAWH3002821, BRAWH3003522, BRAWH3003555, BRAWH3003727, BRAWH3003801, BRAWH3003992, BRAWH3004453, BRAWH3004666, BRAWH3005132, BRAWH3005422, BRAWH3005912, BRAWH3005981, BRAWH3006548, BRAWH3006792, BRAWH3007221, BRAWH3007506, BRAWH3007592, BRAWH3007726, BRAWH3007783, BRAWH3008341, BRAWH3008697, BRAWH3008931, BRAWH3009297, BRCAN2002562, BRCAN2002856, BRCAN2002944, BRCAN2002948, BRCAN2003703, BRCAN2003746, BRCAN2003987, BRCAN2004355, BRCAN2005436, BRCAN2006063, BRCAN2006290, BRCAN2006297, BRCAN2006450, BRCAN2007144, BRCAN2007409, BRCAN2007426, BRCAN2008528, BRCAN2009203, BRCAN2009432, BRCAN2010376, BRCAN2011254, BRCAN2011602, BRCAN2012355, BRCAN2012481, BRCAN2013655, BRCAN2013660, BRCAN2014143, BRCAN2014602, BRCAN2014881, BRCAN2015371, BRCAN2015464, BRCAN2016433, BRCAN2016619, BRCAN2017442, BRCAN2017717, BRCAN2017905, BRCAN2018935, BRCAN2019387, BRCAN2020710, BRCAN2021028, BRCAN2024451, BRCAN2024563, BRCAN2025712, BRCAN2028355, BRCOC2000670, BRCOC2003213, BRCOC2007034, BRCOC2014033, BRCOC2016525, BRCOC2019934, BRCOC2020142, BRHIP2000691, BRHIP2000819, BRHIP2000826, BRHIP2000920, BRHIP2001074, BRHIP2001805, BRHIP2001927, BRHIP2002122, BRHIP2002172, BRHIP2002346, BRHIP2003242, BRHIP2003786, BRHIP2003917, BRHIP2004312, BRHIP2004359, BRHIP2004814, BRHIP2004883, BRHIP2005236, BRHIP2005354, BRHIP2005600, BRHIP2005719, BRHIP2005752, BRHIP2005932, BRHIP2006800, BRHIP2007616, BRHIP2007741, BRHIP2009340, BRHIP2009414, BRHIP2009474, BRHIP2013699, BRHIP2014228, BRHIP2021615, BRHIP2022221, BRHIP2024146, BRHIP2024165, BRHIP2026061, BRHIP2026288, BRHIP2029176, BRHIP2029393, BRHIP3000339, BRHIP3000526, BRHIP3001283, BRHIP3006683, BRHIP3007483, BRHIP3007586, BRHIP3008183, BRHIP3008313, BRHIP3008344, BRHIP3008405, BRHIP3008565, BRHIP3008598, BRHIP3008997, BRHIP3009099, BRHIP3009448, BRHIP3011241, BRHIP3013765, BRHIP3013897, BRHIP3015751, BRHIP3016213, BRHIP3018797, BRHIP3020182, BRHIP3024118, BRHIP3024533, BRHIP3024725, BRHIP3025161, BRHIP3025702, BRHIP3026097, BRHIP3027137, BRHIP3027854, BRSSN2000684, BRSSN2003086, BRSSN2004496, BRSSN2004719, BRSSN2006892, BRSSN2008549, BRSSN2008797, BRSSN2011262, BRSSN2011738, BRSSN2013874, BRSSN2014299, BRSSN2014424, BRSSN2014556, BRSSN2018581, BRSSN2018925, BRSTN2000872, BRSTN2001067, BRSTN2001613, BRSTN2002400, BRSTN2003835, BRSTN2004863, BRSTN2004987, BRSTN2005721, BRSTN2006865, BRSTN2007000, BRSTN2007284, BRSTN2008052, BRSTN2008283, BRSTN2008418, BRSTN2008457, BRSTN2010363, BRSTN2010500, BRSTN2010750, BRSTN2012320, BRSTN2012380, BRSTN2015015, BRSTN2016470, BRSTN2016678, BRSTN2017237, BRSTN2017771, BRSTN2018083, BRSTN2019129, BRTHA1000311, BRTHA2000855, BRTHA2001462, BRTHA2002115, BRTHA2002281, BRTHA2002376, BRTHA2002442, BRTHA2002493, BRTHA2002608, BRTHA2002808, BRTHA2003030, BRTHA2003110, BRTHA2003116, BRTHA2003461, BRTHA2004821, BRTHA2004978, BRTHA2005579, BRTHA2005956, BRTHA2006075, BRTHA2006146, BRTHA2006194, BRTHA2007122, BRTHA2007422, BRTHA2007603, BRTHA2008316, BRTHA2008335, BRTHA2008527, BRTHA2008535, BRTHA2008955, BRTHA2009311, BRTHA2009846, BRTHA2009972, BRTHA2010073, BRTHA2010608, BRTHA2010884, BRTHA2010907, BRTHA2011194, BRTHA2011351, BRTHA2011500, BRTHA2011641, BRTHA2012392, BRTHA2012562, BRTHA2012980, BRTHA2013262, BRTHA2013460, BRTHA2013707, BRTHA2014792, BRTHA2014828, BRTHA2015406, BRTHA2015478, BRTHA2015696, BRTHA2015878, BRTHA2016215, BRTHA2016496, BRTHA2016543, BRTHA2017353, BRTHA2017985, BRTHA2018165, BRTHA2018344, BRTHA2018591, BRTHA2018624, BRTHA2018707, BRTHA2019014, BRTHA2019022, BRTHA2019048, BRTHA3000273, BRTHA3000297, BRTHA3000633, BRTHA3001721, BRTHA3002401, BRTHA3002427, BRTHA3002933, BRTHA3003074, BRTHA3003343, BRTHA3003449, BRTHA3003474, BRTHA3003490, BRTHA3004475, BRTHA3005046, BRTHA3006856, BRTHA3007113, BRTHA3007148, BRTHA3007319, BRTHA3007769, BRTHA3008143, BRTHA3008310, BRTHA3008386, BRTHA3008520, BRTHA3008778, BRTHA3009037, BRTHA3009090, BRTHA3009291, BRTHA3010366, BRTHA3013884, BRTHA3015815, BRTHA3015910, BRTHA3016845, BRTHA3016917, BRTHA3017047, BRTHA3017589, BRTHA3017848, BRTHA3018514, BRTHA3018617, BRTHA3018656, BRTHA3019105, COLON2001721, CTONG1000087, CTONG1000088, CTONG1000467, CTONG2000042, CTONG2008233, CTONG2009423, CTONG2017500, CTONG2019248, CTONG2019788, CTONG2020522, CTONG2023021, CTONG2028124, CTONG3000657, CTONG3001123, CTONG3001370, CTONG3002412, CTONG3004072, CTONG3005813, CTONG3008894, CTONG3009028, CTONG3009239, CTONG3009328, DFNES2000146, DFNES2011239, DFNES2011499, FCBBF1000297, FCBBF2001183, FCBBF3001977, FCBBF3002163, FCBBF3003435, FCBBF3004502, FCBBF3004847, FCBBF3006171, FCBBF3007242, FCBBF3007540, FCBBF3008944, FCBBF3009888, FCBBF3012170, FCBBF3012288, FCBBF3013307, FCBBF3013846, FCBBF3021576, FCBBF3021940, FCBBF3023443, FCBBF3023895, FCBBF3025730, FCBBF3027717, FCBBF4000076, FEBRA1000030, FEBRA2000253, FEBRA2006396, FEBRA2007544, FEBRA2007708, FEBRA2007793, FEBRA2007801, FEBRA2008287, FEBRA2008311, FEBRA2008360, FEBRA2008468, FEBRA2010719, FEBRA2014213, FEBRA2015588, FEBRA2020484, FEBRA2020582, FEBRA2020668, FEBRA2020886, FEBRA2021339, FEBRA2021571, FEBRA2021908, FEBRA2021966, FEBRA2024136, FEBRA2024150, FEBRA2024343, FEBRA2024744, FEBRA2025427, FEBRA2026984, FEBRA2027082, FEBRA2027297, FEBRA2027352, FEBRA2028366, FEBRA2028477, FEBRA2028618, HCASM2007047, HCHON2000028, HCHON2000212, HCHON2000244, HCHON2000626, HCHON2001084, HCHON2001217, HCHON2002676, HCHON2005921, HCHON2006250, HCHON2007881, HCHON2008112, HEART1000074, HEART2007031, HHDPC1000118, HLUNG2001996, HLUNG2002465, HLUNG2003003, HSYRA2009075, IMR322000127, IMR322000917, IMR322001380, IMR322002035, KIDNE2000665, KIDNE2002252, KIDNE2005543, KIDNE2006580, KIDNE2011314, MESAN2006563, MESAN2012054, MESAN2015515, MESTC1000042, NB9N41000340, NESOP2001752, NHNPC2001223, NOVAR2001783, NT2NE2005890, NT2NE2006909, NT2NE2008060, NT2RI2003993, NT2RI2005166, NT2RI2008724, NT2RI2012659, NT2RI2014733, NT2RI2018311, NT2RI2019751, NT2RI3000622, NT2RI3001515, NT2RI3002842, NT2RI3002892, NT2RI3003382, NT2RI3004510, NT2RI3005403, NT2RI3005724, NT2RI3006284, NT2RI3006673, NT2RI3007291, NT2RI3007543, NT2RI3008055, NT2RP7004123, NT2RP7005529, NT2RP7009030, NT2RP7009147, NT2RP7010599, NT2RP7014005, NT2RP7015512, NT2RP7017474, NTONG2000413, NTONG2005969, NTONG2008088, OCBBF1000254, OCBBF2001794, OCBBF2002124, OCBBF2003819, OCBBF2004826, OCBBF2004883, OCBBF2005428, OCBBF2006005, OCBBF2006058, OCBBF2006151, OCBBF2006567, OCBBF2006764, OCBBF2007028, OCBBF2007068, OCBBF2007114, OCBBF2007428, OCBBF2007478, OCBBF2007610, OCBBF2008770, OCBBF2009788, OCBBF2009926, OCBBF2010140, OCBBF2010416, OCBBF2017516, OCBBF2019327, OCBBF2019823, OCBBF2020343, OCBBF2020453, OCBBF2020639, OCBBF2020741, OCBBF2020801, OCBBF2020838, OCBBF2021020, OCBBF2021286, OCBBF2021323, OCBBF2021788, OCBBF2022351, OCBBF2022574, OCBBF2023162, OCBBF2023643, OCBBF2024719, OCBBF2024781, OCBBF2024850, OCBBF2025028, OCBBF2025458, OCBBF2025527, OCBBF2025730, OCBBF2026645, OCBBF2027423, OCBBF2027478, OCBBF2028173, OCBBF2028935, OCBBF2029901, OCBBF2030354, OCBBF2030517, OCBBF2030574, OCBBF2030708, OCBBF2031167, OCBBF2031366, OCBBF2032590, OCBBF2032599, OCBBF2032611, OCBBF2032671, OCBBF2033869, OCBBF2035110, OCBBF2035214, OCBBF2035564, OCBBF2035885, OCBBF2035916, OCBBF2036476, OCBBF2036743, OCBBF2037068, OCBBF2037340, OCBBF2037398, OCBBF2037547, OCBBF2037598, OCBBF2037638, OCBBF2038317, OCBBF3000296, OCBBF3000483, OCBBF3002553, OCBBF3002600, OCBBF3003320, OCBBF3003592, OCBBF3004314, OCBBF3006802, OCBBF3007516, OCBBF3008230, OCBBF3009279, PEBLM2004666, PERIC2000889, PERIC2002766, PERIC2003720, PLACE6001185, PLACE6019385, PUAEN2002489, PUAEN2005930, PUAEN2006701, PUAEN2007044, PUAEN2009174, PUAEN2009655, RECTM2001347, SKMUS2000757, SKNMC2002402, SKNSH2000482, SMINT2001818, SPLEN2001599, SPLEN2002467, SPLEN2006122, SPLEN2010912, SPLEN2012624, SPLEN2025491, SPLEN2027268, SPLEN2028914, SPLEN2029912, SPLEN2031424, SPLEN2031547, SPLEN2032154, SPLEN2034781, SPLEN2036821, SPLEN2036932, SPLEN2037194, SPLEN2038345, SPLEN2042303, SYNOV1000374, SYNOV2005216, SYNOV2014400, SYNOV4002346, SYNOV4002883, SYNOV4007430, SYNOV4007671, SYNOV4008440, TESOP2001605, TESOP2002273, TESOP2002451, TESOP2002950, TESTI1000330, TESTI2003573, TESTI2009474, TESTI2049246, TESTI4000014, TESTI4000209, TESTI4000349, TESTI4001100, TESTI4001561, TESTI4002290, TESTI4002647, TESTI4005857, TESTI4006137, TESTI4006326, TESTI4008797, TESTI4009286, TESTI4010377, TESTI4010851, TESTI4010928, TESTI4011161, TESTI4012702, TESTI4013675, TESTI4013817, TESTI4014159, TESTI4014175, TESTI4014306, TESTI4014694, TESTI4014818, TESTI4019843, TESTI4021478, TESTI4022936, TESTI4024420, TESTI4027821, TESTI4029836, TESTI4037156, TESTI4037188, TESTI4046819, THYMU2001090, THYMU2011736, THYMU2014353, THYMU2016204, THYMU2016523, THYMU2023967, THYMU2025707, THYMU2030264, THYMU2031341, THYMU2031890, THYMU2032696, THYMU2032825, THYMU2033308, THYMU2033787, THYMU2034374, THYMU2035735, THYMU2037226, THYMU2039315, THYMU2039780, THYMU2040975, THYMU3001083, THYMU3001234, THYMU3001379, THYMU3003309, THYMU3004835, THYMU3006485, THYMU3007137, THYMU3008171, TKIDN2009641, TKIDN2009889, TKIDN2010934, TKIDN2013287, TKIDN2015788, TLIVE2001327, TLIVE2004320, TRACH1000205, TRACH2001443, TRACH2001549, TRACH2001684, TRACH2005811, TRACH2006049, TRACH2007834, TRACH2008300, TRACH2023299, TRACH2025344, TRACH2025535, TRACH2025911, TRACH3000014, TRACH3001427, TRACH3002192, TRACH3004068, TRACH3004721, TRACH3005294, TRACH3006038, TRACH3006412, TRACH3007479, TRACH3008093, TRACH3009455, TSTOM1000135, TUTER1000122, TUTER2000904, UTERU2002410, UTERU2004929, UTERU2005621, UTERU2006115, UTERU2007520, UTERU2014678, UTERU2019706, UTERU2019940, UTERU2021163, UTERU2023039, UTERU2023175, UTERU2026203, UTERU2030213, UTERU2030280, UTERU3000226, UTERU3000899, UTERU3001571, UTERU3001572, UTERU3001766, UTERU3003135, UTERU3004709, UTERU3005230, UTERU3005460, UTERU3005907, UTERU3005970, UTERU3006308, UTERU3007419, UTERU3007640, UTERU3007913, UTERU3009259, UTERU3009517, UTERU3009871

ADRGL2000042, BLADE2006830, BRACE2002589, BRACE2003609, BRACE2009318, BRACE2011677, BRACE2029396, BRACE2037299, BRACE2039823, BRACE2039832, BRACE2043105, BRACE3001058, BRACE3001113, BRACE3003026, BRACE3003053, BRACE3009127, BRACE3010076, BRACE3015829, BRACE3021148, BRALZ2017844, BRAMY2019111, BRAMY2035070, BRAMY2035449, BRAMY2035718, BRAMY2038516, BRAMY2039341, BRAMY2040159, BRAMY2041434, BRAMY2045471, BRAMY3004800, BRAWH1000369, BRAWH2006207, BRAWH2006395, BRAWH2008993, BRAWH2009393, BRAWH2010552, BRAWH3007441, BRAWH3009017, BRCAN2002473, BRCAN2002854, BRCAN2003070, BRCAN2014229, BRCOC2019841, BRHIP2002722, BRHIP2003272, BRHIP2005271, BRHIP2005724, BRHIP2006617, BRHIP2008389, BRHIP2012360, BRHIP2017553, BRHIP2026877, BRHIP3000017, BRHIP3000240, BRHIP3008314, BRHIP3026052, BRSTN2013354, BRTHA2002133, BRTHA2002702, BRTHA2007060, BRTHA2010033, BRTHA2011321, BRTHA2013426, BRTHA2013610, BRTHA2016318, BRTHA2017364, BRTHA2017972, BRTHA2018011, BRTHA2018443, BRTHA3000296, BRTHA3003000, BRTHA3008826, CTONG2008721, CTONG2020374, CTONG2020378, CTONG2020411, CTONG2024031, CTONG3004726, FCBBF1000509, FCBBF3010361, FCBBF3027854, FEBRA2000790, FEBRA2001990, FEBRA2006519, FEBRA2008692, FEBRA2014122, FEBRA2027609, FEBRA2028516, HCASM2003018, HCHON2000508, HCHON2000743, HCHON2004858, HSYRA2005628, IMR322001879, NT2RI2009583, NT2RP8000521, OCBBF2003327, OCBBF2005433, OCBBF2006987, OCBBF2008144, OCBBF2009583, OCBBF2011669, OCBBF2019684, OCBBF2020048, OCBBF2030116, OCBBF2032274, OCBBF2034637, OCBBF3002654, OCBBF3003761, OCBBF3004972, PERIC2007068, PUAEN2006335, SPLEN2016932, SPLEN2039379, SYNOV2006620, SYNOV2021953, TESTI1000266, TESTI2015626, TESTI2026647, TESTI4000214, TESTI4001984, TESTI4008058, TESTI4013894, TESTI4015442, TESTI4017714, TESTI4025268, TESTI4025547, TESTI4026207, TESTI4032090, THYMU2004284, THYMU2040925, THYMU3000360, TKIDN2018926, TLIVE2002046, TRACH3000134, UTERU2008040, UTERU2011220, UTERU2021820, UTERU2028734

The result of comparative analysis of cDNA libraries derived from fetal heart (FEHRT) and adult heart (HEART) (Table 22) showed that the genes whose expression levels were different between the two were 34 and two clones as described below.

BRAMY2040592, BRAWH2001671, BRSTN2016470, CTONG2017500, CTONG2028124, CTONG3000657, D3OST3000169, FEBRA2008287, HCHON2000244, HCHON2000626, HEART1000010, HEART1000074, HEART1000088, HEART1000139, HEART2001680, HEART2001756, HEART2006131, HEART2006909, HEART2007031, HEART2010391, HEART2010492, HEART2010495, KIDNE2000665, NB9N41000340, NT2RI2003993, NT2RI3002892, OCBBF2024850, SKMUS2006394, SMINT2001818, TESTI4000209, TKIDN2015788, TRACH3002192, TRACH3005294, TRACH3007479 HEART2009680, THYMU2004284

The result of comparative analysis of cDNA libraries derived from fetal kidney (FEKID) and adult kidney (KIDNE) (Table 23) showed that the genes whose expression levels were different between the two were 40 and two clones as described below.

BRACE2043665, BRACE3010428, BRSTN2016470, CTONG1000087, CTONG2028124, CTONG3008894, HCASM2003415, HCHON2000244, HEART1000074, HHDPC1000118, KIDNE1000064, KIDNE2000665, KIDNE2000722, KIDNE2000832, KIDNE2000846, KIDNE2001361, KIDNE2001847, KIDNE2002252, KIDNE2002991, KIDNE2003837, KIDNE2005543, KIDNE2006580, KIDNE2010264, KIDNE2011314, KIDNE2011532, KIDNE2011635, KIDNE2012945, KIDNE2013095, PEBLM2004666, PLACE6019385, STOMA1000189, SYNOV4007671, TBAES2001258, TESOP2002451, TESTI4000014, TESTI4012702, THYMU2032014, TRACH2001684, TRACH2007834, UTERU2023175 NT2RI2009583, OCBBF2008144

The result of comparative analysis of cDNA libraries derived from fetal lung (FELNG) and adult lung (HLUNG) (Table 24) showed that the genes whose expression levels were different between the two were 51 and eight clones as described below.

BRAWH3007592, BRCAN2021028, BRHIP2000819, BRSTN2016470, CTONG1000087, CTONG2028124, HCASM2007047, HEART1000074, HLUNG1000017, HLUNG2000014, HLUNG2001996, HLUNG2002465, HLUNG2002958, HLUNG2003003, HLUNG2003872, HLUNG2010464, HLUNG2011041, HLUNG2011298, HLUNG2012049, HLUNG2012287, HLUNG2012727, HLUNG2013204, HLUNG2013304, HLUNG2013622, HLUNG2013851, HLUNG2014262, HLUNG2014288, HLUNG2014449, HLUNG2015617, HLUNG2017350, HLUNG2017546, HLUNG2017806, HLUNG2019058, HSYRA2008376, KIDNE2012945, NT2RI2003993, NT2RI3007543, OCBBF3000483, SMINT1000192, SPLEN2028914, SPLEN2031547, STOMA1000189, SYNOV4007671, TESOP1000127, TESTI2003573, TESTI4000014, TESTI4037156, TRACH2005811, TRACH3004068, UTERU2005621, UTERU2023175

FEBRA2028516, HCHON2000508, HLUNG2013350, HLUNG2015418, HLUNG2015548, HLUNG2016862, TESTI2015626, TRACH2019672

These genes are involved in regeneration of tissues and/or cells.

A nucleotide sequence information-based analysis was carried out to identify the genes whose expression frequencies are higher or lower in CD34+ cell (cell expressing a glycoprotein CD34) treated with the osteoclast differentiation factor (Molecular Medicine 38. 642-648. (2001)) than in the untreated CD34+ cell, which is the precursor cell of monocyte/macrophage line. The result of comparative analysis for the frequency between the two cDNA libraries prepared from the RNA of CD34+ cells (CD34C) and from the RNA of CD34+ cells treated with the osteoclast differentiation factor (D30ST, D60ST or D90ST) showed following genes whose expression levels were different between the two.

TABLE 2

| Clone ID | CD34C | D30ST | D60ST | D90ST |
|---|---|---|---|---|
| BRACE3013780 | 0.000 | 55.996 | 0.000 | 0.000 |
| BRAMY2047420 | 42.545 | 0.000 | 0.000 | 0.000 |
| BRSTN2016470 | 0.000 | 2.555 | 0.000 | 0.000 |
| CTONG3008894 | 0.000 | 8.487 | 0.000 | 0.000 |
| D30ST2002182 | 0.000 | 86.773 | 0.000 | 0.000 |
| D30ST2002648 | 0.000 | 17.515 | 0.000 | 0.000 |
| D30ST3000169 | 20.553 | 28.566 | 0.000 | 19.796 |
| PEBLM2005183 | 0.000 | 0.000 | 0.000 | 50.747 |
| PUAEN2009655 | 0.000 | 0.000 | 0.000 | 49.285 |
| TESTI4000014 | 7.500 | 0.000 | 0.000 | 0.000 |
| TESTI4010851 | 0.000 | 0.000 | 0.000 | 7.372 |
| TRACH2023299 | 0.000 | 74.521 | 0.000 | 0.000 |
| TRACH2025535 | 0.000 | 6.778 | 0.000 | 0.000 |
| TRACH3001427 | 0.000 | 0.000 | 0.000 | 12.519 |
| UTERU2006137 | 0.000 | 90.731 | 0.000 | 0.000 |
| HCHON2000508 | 0.000 | 4.360 | 50.138 | 0.000 |
| TESTI2015626 | 0.000 | 0.000 | 0.000 | 4.435 |

A survey was performed for genes whose expression levels are varied in response to induction of differentiation (stimulation by retinoic acid (RA) or growth inhibitor treatment after RA stimulation) in cultured cells of a neural strain, NT2. The result of comparative analysis of cDNA libraries derived from undifferentiated NT2 cells (NT2RM) and the cells subjected to the differentiation treatment (NT2RP, NT2R1 or NT2NE) showed following genes whose expression levels were different between the two.

TABLE 3

| Clone ID | NT2RM | NT2RP | NT2RI | NT2NE |
|---|---|---|---|---|
| BNGH42007788 | 0.000 | 7.419 | 0.000 | 0.000 |
| BRACE1000186 | 0.000 | 5.211 | 0.000 | 7.836 |
| BRACE2006319 | 0.000 | 8.450 | 0.000 | 0.000 |
| BRACE2014306 | 0.000 | 0.000 | 0.000 | 17.640 |
| BRACE2015058 | 0.000 | 10.567 | 0.000 | 0.000 |
| BRACE2044286 | 0.000 | 0.000 | 33.923 | 0.000 |
| BRACE3010428 | 0.000 | 0.000 | 2.080 | 0.000 |
| BRAMY2044078 | 0.000 | 10.567 | 0.000 | 0.000 |
| BRAWH2014645 | 0.000 | 7.643 | 1.921 | 0.000 |
| BRAWH2014662 | 0.000 | 0.000 | 0.000 | 56.250 |
| BRAWH3002574 | 0.000 | 0.000 | 12.014 | 0.000 |
| BRAWH3003992 | 0.000 | 34.956 | 0.000 | 0.000 |
| BRAWH3005981 | 0.000 | 70.676 | 0.000 | 0.000 |
| BRAWH3007592 | 0.000 | 8.644 | 3.259 | 0.000 |
| BRCAN2009432 | 0.000 | 3.173 | 0.000 | 0.000 |
| BRCAN2016619 | 0.000 | 0.000 | 23.940 | 0.000 |
| BRCAN2028355 | 0.000 | 0.000 | 0.000 | 5.354 |
| BRHIP2001074 | 0.000 | 47.218 | 0.000 | 0.000 |
| BRHIP2007741 | 0.000 | 0.000 | 6.309 | 6.291 |
| BRHIP2014228 | 0.000 | 35.475 | 0.000 | 0.000 |
| BRHIP2024146 | 0.000 | 0.000 | 1.106 | 0.000 |
| BRHIP3007586 | 0.000 | 0.000 | 0.000 | 12.507 |
| BRHIP3018797 | 0.000 | 0.000 | 4.869 | 0.000 |
| BRTHA2003461 | 0.000 | 0.000 | 3.989 | 0.000 |
| BRTHA3000633 | 0.000 | 0.000 | 62.102 | 0.000 |
| BRTHA3003490 | 0.000 | 3.913 | 0.000 | 0.000 |
| COLON2001721 | 0.000 | 0.000 | 10.053 | 0.000 |
| CTONG1000087 | 0.000 | 5.041 | 5.701 | 3.790 |
| CTONG2008233 | 0.000 | 8.275 | 2.496 | 7.466 |
| CTONG2020638 | 0.000 | 0.000 | 22.499 | 0.000 |
| CTONG2028124 | 0.000 | 1.211 | 0.913 | 0.000 |
| CTONG3003905 | 0.000 | 47.197 | 0.000 | 0.000 |
| CTONG3008894 | 0.000 | 7.008 | 9.247 | 2.634 |
| CTONG3009028 | 0.000 | 0.000 | 2.924 | 0.000 |
| CTONG3009239 | 0.000 | 0.000 | 2.624 | 0.000 |
| DFNES2011499 | 0.000 | 0.000 | 22.548 | 0.000 |
| FCBBF3001977 | 0.000 | 17.952 | 13.536 | 0.000 |
| FEBRA1000030 | 0.000 | 0.000 | 0.000 | 59.247 |
| FEBRA2006396 | 0.000 | 0.000 | 14.606 | 0.000 |
| FEBRA2007801 | 0.000 | 5.950 | 0.000 | 0.000 |
| HCHON2000028 | 0.000 | 0.000 | 5.766 | 0.000 |
| HCHON2000244 | 0.000 | 3.171 | 1.195 | 0.000 |
| HCHON2001084 | 0.000 | 0.000 | 4.173 | 0.000 |
| HCHON2001217 | 0.000 | 2.509 | 5.674 | 0.000 |
| HCHON2001548 | 0.000 | 27.583 | 0.000 | 0.000 |
| HCHON2006250 | 0.000 | 3.771 | 0.000 | 0.000 |
| HEART1000074 | 0.000 | 1.830 | 0.000 | 0.000 |
| HHDPC1000118 | 0.000 | 10.102 | 3.809 | 15.191 |
| HSYRA2009075 | 0.000 | 2.899 | 0.000 | 0.000 |
| IMR322000127 | 0.000 | 3.733 | 0.000 | 0.000 |
| IMR322001380 | 0.000 | 0.000 | 3.602 | 7.184 |
| KIDNE2000665 | 0.000 | 0.000 | 0.000 | 7.016 |
| KIDNE2002252 | 0.000 | 0.000 | 3.961 | 0.000 |
| MESAN2006563 | 0.000 | 1.664 | 2.510 | 0.000 |
| MESAN2012054 | 0.000 | 0.000 | 4.636 | 0.000 |
| MESAN2015515 | 0.000 | 6.402 | 0.000 | 0.000 |
| NT2NE2003252 | 0.000 | 0.000 | 0.000 | 100.000 |
| NT2NE2005890 | 0.000 | 0.000 | 0.000 | 64.265 |
| NT2NE2006531 | 0.000 | 0.000 | 0.000 | 100.000 |
| NT2NE2006909 | 0.000 | 0.000 | 0.000 | 1.034 |
| NT2NE2008060 | 0.000 | 0.000 | 0.000 | 74.472 |
| NT2RI2003993 | 0.000 | 0.000 | 6.899 | 0.000 |
| NT2RI2004618 | 0.000 | 0.000 | 100.000 | 0.000 |
| NT2RI2005166 | 0.000 | 0.000 | 47.393 | 0.000 |
| NT2RI2006686 | 0.000 | 0.000 | 21.246 | 0.000 |
| NT2RI2008724 | 0.000 | 0.000 | 3.337 | 0.000 |
| NT2RI2009855 | 0.000 | 0.000 | 100.000 | 0.000 |
| NT2RI2011422 | 0.000 | 0.000 | 100.000 | 0.000 |
| NT2RI2011683 | 0.000 | 0.000 | 100.000 | 0.000 |
| NT2RI2012659 | 0.000 | 0.000 | 24.645 | 0.000 |
| NT2RI2012990 | 0.000 | 0.000 | 24.360 | 0.000 |
| NT2RI2013357 | 0.000 | 0.000 | 73.545 | 0.000 |
| NT2RI2014247 | 0.000 | 0.000 | 27.718 | 0.000 |
| NT2RI2014551 | 0.000 | 0.000 | 100.000 | 0.000 |
| NT2RI2014733 | 0.000 | 0.000 | 45.319 | 0.000 |
| NT2RI2016128 | 0.000 | 0.000 | 100.000 | 0.000 |
| NT2RI2018311 | 0.000 | 0.000 | 78.082 | 0.000 |
| NT2RI2018883 | 0.000 | 0.000 | 49.384 | 0.000 |
| NT2RI2019751 | 0.000 | 0.000 | 71.913 | 0.000 |
| NT2RI2023303 | 0.000 | 0.000 | 100.000 | 0.000 |
| NT2RI2025909 | 0.000 | 0.000 | 100.000 | 0.000 |
| NT2RI2025957 | 0.000 | 0.000 | 100.000 | 0.000 |
| NT2RI2027081 | 0.000 | 0.000 | 100.000 | 0.000 |
| NT2RI2027396 | 0.000 | 0.000 | 100.000 | 0.000 |
| NT2RI3000622 | 0.000 | 0.000 | 15.558 | 0.000 |
| NT2RI3001263 | 0.000 | 0.000 | 100.000 | 0.000 |
| NT2RI3001515 | 0.000 | 0.000 | 19.682 | 0.000 |
| NT2RI3002303 | 0.000 | 0.000 | 100.000 | 0.000 |
| NT2RI3002842 | 0.000 | 45.164 | 34.054 | 0.000 |
| NT2RI3002892 | 0.000 | 4.241 | 3.198 | 0.000 |
| NT2RI3003031 | 0.000 | 0.000 | 100.000 | 0.000 |
| NT2RI3003095 | 0.000 | 0.000 | 100.000 | 0.000 |
| NT2RI3003162 | 0.000 | 0.000 | 100.000 | 0.000 |
| NT2RI3003382 | 0.000 | 0.000 | 71.913 | 0.000 |
| NT2RI3003409 | 0.000 | 0.000 | 100.000 | 0.000 |
| NT2RI3004381 | 0.000 | 0.000 | 100.000 | 0.000 |
| NT2RI3004510 | 0.000 | 0.000 | 51.230 | 0.000 |
| NT2RI3005202 | 0.000 | 0.000 | 100.000 | 0.000 |
| NT2RI3005403 | 0.000 | 0.000 | 62.102 | 0.000 |
| NT2RI3005724 | 0.000 | 14.284 | 10.770 | 0.000 |
| NT2RI3006132 | 0.000 | 0.000 | 100.000 | 0.000 |
| NT2RI3006171 | 0.000 | 0.000 | 100.000 | 0.000 |
| NT2RI3006284 | 0.000 | 0.000 | 3.760 | 0.000 |
| NT2RI3006340 | 0.000 | 0.000 | 14.839 | 0.000 |
| NT2RI3006376 | 0.000 | 0.000 | 100.000 | 0.000 |
| NT2RI3006673 | 0.000 | 0.000 | 18.135 | 0.000 |
| NT2RI3006796 | 0.000 | 0.000 | 100.000 | 0.000 |
| NT2RI3007065 | 0.000 | 0.000 | 100.000 | 0.000 |
| NT2RI3007158 | 0.000 | 0.000 | 100.000 | 0.000 |
| NT2RI3007291 | 0.000 | 0.000 | 22.746 | 0.000 |
| NT2RI3007543 | 0.000 | 0.000 | 1.542 | 6.150 |
| NT2RI3007757 | 0.000 | 30.480 | 45.964 | 0.000 |
| NT2RI3007978 | 0.000 | 0.000 | 100.000 | 0.000 |
| NT2RI3008055 | 0.000 | 0.000 | 40.142 | 0.000 |
| NT2RI3008162 | 0.000 | 0.000 | 100.000 | 0.000 |
| NT2RI3008652 | 0.000 | 0.000 | 100.000 | 0.000 |
| NT2RI3008697 | 0.000 | 0.000 | 100.000 | 0.000 |
| NT2RI3008974 | 0.000 | 0.000 | 100.000 | 0.000 |
| NT2RI3009158 | 0.000 | 0.000 | 45.727 | 0.000 |
| NT2RP7000359 | 0.000 | 100.000 | 0.000 | 0.000 |
| NT2RP7000466 | 0.000 | 2.098 | 3.164 | 0.000 |
| NT2RP7004027 | 0.000 | 100.000 | 0.000 | 0.000 |
| NT2RP7004123 | 0.000 | 4.625 | 0.000 | 0.000 |
| NT2RP7005118 | 0.000 | 100.000 | 0.000 | 0.000 |
| NT2RP7005529 | 0.000 | 35.588 | 0.000 | 0.000 |
| NT2RP7005846 | 0.000 | 100.000 | 0.000 | 0.000 |
| NT2RP7009030 | 0.000 | 46.373 | 0.000 | 0.000 |
| NT2RP7009147 | 0.000 | 7.679 | 2.895 | 0.000 |
| NT2RP7009867 | 0.000 | 100.000 | 0.000 | 0.000 |
| NT2RP7010128 | 0.000 | 100.000 | 0.000 | 0.000 |
| NT2RP7010599 | 0.000 | 77.250 | 0.000 | 0.000 |
| NT2RP7011570 | 0.000 | 100.000 | 0.000 | 0.000 |
| NT2RP7013795 | 0.000 | 10.432 | 0.000 | 0.000 |
| NT2RP7014005 | 0.000 | 14.022 | 21.145 | 0.000 |
| NT2RP7015512 | 0.000 | 31.156 | 0.000 | 0.000 |
| NT2RP7017365 | 0.000 | 100.000 | 0.000 | 0.000 |
| NT2RP7017474 | 0.000 | 45.366 | 0.000 | 0.000 |
| NT2RP7017546 | 0.000 | 100.000 | 0.000 | 0.000 |
| NT2RP8000137 | 0.000 | 100.000 | 0.000 | 0.000 |
| NT2RP8000296 | 0.000 | 100.000 | 0.000 | 0.000 |
| NT2RP8000483 | 0.000 | 100.000 | 0.000 | 0.000 |
| NTONG2005969 | 0.000 | 15.484 | 0.000 | 0.000 |
| OCBBF2007028 | 0.000 | 0.000 | 2.509 | 0.000 |
| OCBBF2037068 | 0.000 | 0.000 | 27.189 | 54.224 |
| PLACE7000514 | 0.000 | 0.000 | 8.644 | 0.000 |
| PUAEN2007044 | 0.000 | 3.455 | 13.024 | 0.000 |
| SPLEN2002467 | 0.000 | 7.852 | 0.000 | 0.000 |
| SPLEN2006122 | 0.000 | 0.000 | 1.530 | 0.000 |
| SPLEN2028914 | 0.000 | 6.730 | 10.149 | 0.000 |
| SPLEN2031547 | 0.000 | 0.000 | 2.861 | 0.000 |
| SYNOV4002346 | 0.000 | 0.000 | 10.899 | 0.000 |
| SYNOV4007671 | 42.189 | 0.000 | 0.000 | 2.657 |
| SYNOV4008440 | 0.000 | 0.000 | 2.681 | 0.000 |
| TESOP2002273 | 0.000 | 9.782 | 0.000 | 0.000 |
| TESTI2003573 | 0.000 | 0.000 | 13.573 | 0.000 |
| TESTI4000014 | 0.000 | 3.443 | 0.974 | 0.000 |
| TESTI4009286 | 0.000 | 1.747 | 0.000 | 0.000 |
| TESTI4010851 | 0.000 | 5.270 | 1.987 | 0.000 |
| TESTI4012702 | 0.000 | 0.000 | 3.961 | 0.000 |
| TESTI4029671 | 0.000 | 44.826 | 0.000 | 0.000 |
| TESTI4037156 | 0.000 | 4.669 | 1.509 | 2.006 |
| THYMU3000133 | 0.000 | 34.767 | 8.738 | 0.000 |
| TRACH1000205 | 0.000 | 0.000 | 7.152 | 0.000 |
| TRACH2005811 | 0.000 | 0.000 | 0.000 | 3.822 |
| TRACH2007834 | 0.000 | 3.647 | 1.833 | 0.000 |
| TRACH2025535 | 0.000 | 1.399 | 3.165 | 4.208 |
| TRACH3001427 | 0.000 | 8.951 | 1.687 | 3.365 |
| TRACH3002192 | 0.000 | 0.000 | 2.267 | 4.520 |
| TRACH3004721 | 0.000 | 14.013 | 5.283 | 10.536 |
| TRACH3008093 | 0.000 | 0.000 | 8.902 | 0.000 |
| TRACH3008535 | 0.000 | 68.270 | 0.000 | 0.000 |
| TRACH3008713 | 0.000 | 68.270 | 0.000 | 0.000 |
| UTERU2002410 | 0.000 | 0.000 | 1.067 | 0.000 |
| UTERU2023175 | 0.000 | 7.176 | 0.000 | 5.396 |
| ADRGL2000042 | 0.000 | 0.000 | 0.000 | 9.204 |
| BRACE2003609 | 0.000 | 21.907 | 0.000 | 32.942 |
| BRACE3003026 | 0.000 | 0.000 | 59.349 | 0.000 |
| BRHIP3000017 | 0.000 | 0.000 | 8.013 | 0.000 |
| CTONG2020411 | 0.000 | 16.593 | 25.022 | 0.000 |
| FCBBF1000509 | 0.000 | 0.000 | 0.000 | 6.762 |
| FCBBF3027854 | 0.000 | 0.000 | 28.447 | 0.000 |
| FEBRA2028516 | 0.000 | 11.027 | 6.236 | 0.000 |
| HCHON2000508 | 0.000 | 0.900 | 0.000 | 0.000 |
| IMR322001879 | 0.000 | 0.000 | 35.028 | 0.000 |
| NT2RI2005772 | 0.000 | 0.000 | 100.000 | 0.000 |
| NT2RI2008952 | 0.000 | 0.000 | 100.000 | 0.000 |
| NT2RI2009583 | 0.000 | 0.000 | 0.813 | 0.811 |
| NT2RI2018448 | 0.000 | 15.176 | 11.442 | 0.000 |
| NT2RI2027157 | 0.000 | 0.000 | 100.000 | 0.000 |
| NT2RI3000174 | 0.000 | 0.000 | 61.866 | 0.000 |
| NT2RI3001132 | 0.000 | 0.000 | 100.000 | 0.000 |
| NT2RI3002557 | 0.000 | 0.000 | 100.000 | 0.000 |
| NT2RI3005928 | 0.000 | 0.000 | 100.000 | 0.000 |
| NT2RI3007167 | 0.000 | 0.000 | 100.000 | 0.000 |
| NT2RI3007443 | 0.000 | 0.000 | 100.000 | 0.000 |
| NT2RP7008435 | 0.000 | 100.000 | 0.000 | 0.000 |
| NT2RP8000521 | 0.000 | 62.933 | 0.000 | 0.000 |
| OCBBF2006987 | 0.000 | 62.306 | 0.000 | 0.000 |
| PERIC2007068 | 0.000 | 3.719 | 2.804 | 0.000 |
| TESTI2015626 | 9.463 | 0.000 | 0.000 | 2.384 |
| TESTI4015442 | 0.000 | 48.593 | 0.000 | 0.000 |
| TLIVE2002046 | 0.000 | 0.000 | 3.298 | 0.000 |
| TRACH3000134 | 0.000 | 43.581 | 0.000 | 0.000 |
| TUTER2000057 | 0.000 | 0.000 | 7.539 | 0.000 |

The result of comparative analysis of cDNA libraries derived from the cerebral cortex of Alzheimer patients (BRALZ and BRASW), and from whole tissues of a normal brain (BRAWH) showed the following genes whose expression levels differed between the two.

TABLE 4

| Clone ID | BRAWH | BRALZ | BRASW |
|---|---|---|---|
| ASTRO1000009 | 2.611 | 0.000 | 0.000 |
| BLADE2008398 | 12.401 | 0.000 | 0.000 |
| BRACE1000186 | 4.324 | 0.000 | 0.000 |
| BRACE1000258 | 31.956 | 0.000 | 0.000 |
| BRACE1000533 | 11.795 | 0.000 | 0.000 |
| BRACE2005457 | 58.488 | 0.000 | 0.000 |
| BRACE2010489 | 63.510 | 0.000 | 0.000 |
| BRACE2014657 | 15.451 | 0.000 | 0.000 |
| BRACE2035381 | 10.177 | 0.000 | 0.000 |
| BRACE2044286 | 18.667 | 0.000 | 0.000 |
| BRACE2045954 | 27.309 | 0.000 | 0.000 |
| BRACE3000787 | 32.844 | 0.000 | 0.000 |
| BRACE3003192 | 58.488 | 0.000 | 0.000 |
| BRACE3005499 | 31.276 | 0.000 | 0.000 |
| BRACE3007480 | 19.471 | 0.000 | 0.000 |
| BRACE3009237 | 18.139 | 0.000 | 0.000 |
| BRACE3009724 | 58.488 | 0.000 | 0.000 |
| BRACE3009747 | 2.237 | 0.000 | 0.000 |
| BRACE3010428 | 6.868 | 0.000 | 0.000 |
| BRACE3011271 | 11.036 | 0.000 | 0.000 |
| BRACE3011421 | 28.251 | 0.000 | 0.000 |
| BRACE3012364 | 8.506 | 0.000 | 0.000 |
| BRACE3022769 | 4.285 | 0.000 | 0.000 |
| BRACE3026735 | 24.173 | 0.000 | 0.000 |
| BRACE3031838 | 58.488 | 0.000 | 0.000 |
| BRALZ2011796 | 5.511 | 39.830 | 0.000 |
| BRALZ2012183 | 0.000 | 100.000 | 0.000 |
| BRALZ2012848 | 0.000 | 100.000 | 0.000 |
| BRALZ2014484 | 0.000 | 100.000 | 0.000 |
| BRALZ2016085 | 0.000 | 100.000 | 0.000 |
| BRALZ2016498 | 0.000 | 100.000 | 0.000 |
| BRALZ2017359 | 0.000 | 75.184 | 0.000 |
| BRAMY2003008 | 26.445 | 0.000 | 0.000 |
| BRAMY2005052 | 11.612 | 0.000 | 0.000 |
| BRAMY2019300 | 49.811 | 0.000 | 0.000 |
| BRAMY2019963 | 20.428 | 0.000 | 0.000 |
| BRAMY2036567 | 7.474 | 0.000 | 0.000 |
| BRAMY2037823 | 29.664 | 0.000 | 0.000 |
| BRAMY2040592 | 3.482 | 12.582 | 0.000 |
| BRAMY3002803 | 14.428 | 0.000 | 0.000 |
| BRAMY3004224 | 33.027 | 0.000 | 0.000 |
| BRAMY3005091 | 19.193 | 0.000 | 0.000 |
| BRASW1000053 | 0.000 | 0.000 | 100.000 |
| BRASW1000125 | 0.000 | 0.000 | 99.054 |
| BRAWH1000127 | 15.983 | 0.000 | 0.000 |
| BRAWH2001395 | 14.290 | 3.037 | 0.000 |
| BRAWH2001671 | 7.605 | 0.000 | 0.000 |
| BRAWH2001940 | 37.398 | 0.000 | 0.000 |
| BRAWH2001973 | 37.398 | 0.000 | 0.000 |
| BRAWH2002560 | 6.454 | 0.000 | 0.000 |
| BRAWH2002761 | 100.000 | 0.000 | 0.000 |
| BRAWH2005315 | 100.000 | 0.000 | 0.000 |
| BRAWH2007658 | 58.101 | 0.000 | 0.000 |
| BRAWH2010000 | 18.745 | 0.000 | 0.000 |
| BRAWH2010084 | 100.000 | 0.000 | 0.000 |
| BRAWH2010536 | 14.718 | 0.000 | 0.000 |
| BRAWH2012162 | 36.060 | 0.000 | 0.000 |
| BRAWH2012326 | 100.000 | 0.000 | 0.000 |
| BRAWH2013294 | 39.442 | 0.000 | 0.000 |
| BRAWH2013871 | 37.485 | 0.000 | 0.000 |
| BRAWH2014414 | 17.865 | 0.000 | 0.000 |
| BRAWH2014645 | 4.228 | 0.000 | 0.000 |
| BRAWH2014662 | 15.521 | 0.000 | 0.000 |
| BRAWH2014876 | 10.473 | 0.000 | 0.000 |
| BRAWH2014954 | 58.488 | 0.000 | 0.000 |
| BRAWH2016221 | 47.417 | 0.000 | 0.000 |
| BRAWH2016439 | 100.000 | 0.000 | 0.000 |
| BRAWH2016702 | 73.807 | 0.000 | 0.000 |
| BRAWH2016724 | 35.119 | 0.000 | 0.000 |
| BRAWH3000078 | 100.000 | 0.000 | 0.000 |
| BRAWH3000100 | 100.000 | 0.000 | 0.000 |
| BRAWH3000314 | 71.553 | 0.000 | 0.000 |
| BRAWH3000491 | 100.000 | 0.000 | 0.000 |
| BRAWH3001326 | 45.606 | 0.000 | 0.000 |
| BRAWH3001475 | 100.000 | 0.000 | 0.000 |
| BRAWH3001891 | 34.539 | 0.000 | 0.000 |
| BRAWH3002574 | 13.222 | 0.000 | 0.000 |
| BRAWH3002600 | 36.800 | 0.000 | 0.000 |
| BRAWH3002819 | 100.000 | 0.000 | 0.000 |
| BRAWH3002821 | 21.953 | 0.000 | 0.000 |
| BRAWH3003522 | 100.000 | 0.000 | 0.000 |
| BRAWH3003555 | 15.229 | 0.000 | 0.000 |
| BRAWH3003727 | 10.055 | 0.000 | 0.000 |
| BRAWH3003801 | 100.000 | 0.000 | 0.000 |
| BRAWH3003992 | 29.008 | 0.000 | 0.000 |
| BRAWH3004453 | 100.000 | 0.000 | 0.000 |
| BRAWH3004666 | 49.499 | 0.000 | 0.000 |
| BRAWH3005132 | 49.811 | 0.000 | 0.000 |
| BRAWH3005422 | 100.000 | 0.000 | 0.000 |
| BRAWH3005912 | 100.000 | 0.000 | 0.000 |
| BRAWH3005981 | 29.324 | 0.000 | 0.000 |
| BRAWH3006548 | 71.018 | 0.000 | 0.000 |
| BRAWH3006792 | 49.499 | 0.000 | 0.000 |
| BRAWH3007221 | 100.000 | 0.000 | 0.000 |
| BRAWH3007506 | 100.000 | 0.000 | 0.000 |
| BRAWH3007592 | 8.966 | 0.000 | 0.000 |
| BRAWH3007726 | 54.530 | 0.000 | 0.000 |
| BRAWH3007783 | 100.000 | 0.000 | 0.000 |
| BRAWH3008341 | 100.000 | 0.000 | 0.000 |
| BRAWH3008697 | 100.000 | 0.000 | 0.000 |
| BRAWH3008931 | 3.463 | 0.000 | 0.000 |
| BRAWH3009297 | 58.488 | 0.000 | 0.000 |
| BRCOC2003213 | 10.381 | 0.000 | 0.000 |
| BRCOC2014033 | 15.633 | 0.000 | 0.000 |
| BRCOC2020142 | 22.014 | 0.000 | 0.000 |
| BRHIP2000920 | 36.630 | 0.000 | 0.000 |
| BRHIP2005719 | 49.499 | 0.000 | 0.000 |
| BRHIP2007741 | 6.943 | 0.000 | 0.000 |
| BRHIP2014228 | 29.439 | 0.000 | 0.000 |
| BRHIP2024146 | 3.042 | 12.091 | 0.000 |
| BRHIP2026288 | 0.000 | 77.982 | 0.000 |
| BRHIP3000339 | 14.290 | 3.037 | 0.000 |
| BRHIP3006683 | 24.100 | 0.000 | 0.000 |
| BRHIP3007586 | 17.255 | 0.000 | 0.000 |
| BRHIP3008405 | 35.187 | 0.000 | 0.000 |
| BRHIP3018797 | 30.810 | 4.840 | 0.000 |
| BRSSN2000684 | 23.433 | 0.000 | 0.000 |
| BRSSN2011738 | 31.553 | 0.000 | 0.000 |
| BRSSN2014299 | 3.695 | 0.000 | 0.000 |
| BRSTN2008052 | 32.844 | 0.000 | 0.000 |
| BRSTN2015015 | 14.017 | 0.000 | 0.000 |
| BRSTN2016470 | 0.438 | 7.909 | 0.000 |
| BRTHA1000311 | 11.803 | 0.000 | 0.000 |
| BRTHA2008335 | 16.281 | 0.000 | 0.000 |
| BRTHA3002427 | 8.577 | 0.000 | 0.000 |
| BRTHA3003490 | 1.623 | 0.000 | 0.000 |
| BRTHA3008520 | 47.417 | 0.000 | 0.000 |
| BRTHA3017848 | 47.417 | 0.000 | 0.000 |
| COLON2001721 | 11.065 | 0.000 | 0.000 |
| CTONG2017500 | 2.649 | 0.000 | 0.000 |
| CTONG2028124 | 0.503 | 0.000 | 0.000 |
| CTONG3000657 | 3.880 | 0.000 | 0.000 |
| CTONG3001123 | 7.847 | 0.000 | 0.000 |
| CTONG3009328 | 11.993 | 43.334 | 0.000 |
| FCBBF2001183 | 16.537 | 5.975 | 0.000 |
| FCBBF3001977 | 7.448 | 0.000 | 0.000 |
| FEBRA2007544 | 14.689 | 0.000 | 0.000 |
| FEBRA2007801 | 4.937 | 0.000 | 0.000 |
| FEBRA2020886 | 12.124 | 0.000 | 0.000 |
| FEBRA2028618 | 5.082 | 0.000 | 0.000 |
| HCASM2007047 | 3.431 | 0.000 | 0.000 |
| HCHON2000244 | 0.658 | 0.000 | 0.000 |
| HCHON2000626 | 2.351 | 0.000 | 0.000 |
| HCHON2001217 | 3.123 | 0.000 | 0.000 |
| HCHON2002676 | 13.647 | 0.000 | 0.000 |
| HCHON2006250 | 1.565 | 0.000 | 0.000 |
| HEART1000074 | 0.759 | 0.000 | 0.000 |
| HHDPC1000118 | 2.096 | 0.000 | 0.000 |
| HLUNG2002465 | 1.209 | 0.000 | 0.000 |
| IMR322000127 | 3.098 | 5.597 | 0.000 |
| IMR322001380 | 0.000 | 7.163 | 0.000 |
| IMR322002035 | 36.176 | 0.000 | 0.000 |
| KIDNE2006580 | 7.013 | 0.000 | 0.000 |
| MESAN2006563 | 0.691 | 0.000 | 0.000 |
| MESAN2012054 | 12.754 | 0.000 | 0.000 |
| MESTC1000042 | 2.245 | 0.000 | 0.000 |
| NOVAR2001783 | 4.027 | 0.000 | 0.000 |
| NT2NE2006909 | 0.285 | 0.000 | 0.000 |
| NT2RI2008724 | 1.836 | 0.000 | 0.000 |
| NT2RI2012659 | 13.562 | 0.000 | 0.000 |
| NT2RI2014733 | 24.938 | 0.000 | 0.000 |
| NT2RI3002892 | 8.799 | 0.000 | 0.000 |
| NT2RI3006284 | 4.138 | 0.000 | 0.000 |
| NT2RI3006673 | 19.959 | 0.000 | 0.000 |
| NT2RI3007543 | 1.697 | 0.000 | 0.000 |
| NT2RI3008055 | 44.179 | 0.000 | 0.000 |
| NT2RP7005529 | 14.766 | 0.000 | 0.000 |
| NT2RP7009147 | 14.337 | 0.000 | 0.000 |
| NT2RP7014005 | 5.818 | 0.000 | 0.000 |
| NT2RP7017474 | 18.823 | 0.000 | 0.000 |
| NTONG2005969 | 0.000 | 11.607 | 0.000 |
| OCBBF2001794 | 4.728 | 0.000 | 0.000 |
| OCBBF2006005 | 9.535 | 0.000 | 0.000 |
| OCBBF2006764 | 15.345 | 0.000 | 0.000 |
| OCBBF2007028 | 9.665 | 0.000 | 0.000 |
| OCBBF2007114 | 0.000 | 38.623 | 0.000 |
| OCBBF2010140 | 32.508 | 0.000 | 0.000 |
| OCBBF2021286 | 18.456 | 0.000 | 0.000 |
| OCBBF2023162 | 0.000 | 37.152 | 0.000 |
| OCBBF2024850 | 4.445 | 0.000 | 0.000 |
| OCBBF2028935 | 5.789 | 4.183 | 0.000 |
| OCBBF2036743 | 11.053 | 0.000 | 0.000 |
| OCBBF2038317 | 19.713 | 0.000 | 0.000 |
| OCBBF3000483 | 11.973 | 0.000 | 0.000 |
| OCBBF3008230 | 29.840 | 0.000 | 0.000 |
| PEBLM2004666 | 3.715 | 0.000 | 0.000 |
| PLACE6001185 | 21.358 | 0.000 | 0.000 |
| PUAEN2005930 | 18.362 | 0.000 | 0.000 |
| PUAEN2006701 | 2.249 | 8.128 | 0.000 |
| PUAEN2007044 | 8.600 | 0.000 | 0.000 |
| PUAEN2009655 | 18.275 | 0.000 | 0.000 |
| SMINT2001818 | 0.000 | 3.387 | 0.000 |
| SPLEN2028914 | 2.792 | 0.000 | 0.000 |
| SPLEN2031424 | 15.229 | 0.000 | 0.000 |
| SPLEN2031547 | 1.574 | 5.689 | 0.000 |
| SPLEN2034781 | 27.984 | 0.000 | 0.000 |
| SPLEN2036932 | 2.932 | 0.000 | 0.000 |
| SYNOV2014400 | 12.977 | 0.000 | 0.000 |
| SYNOV4002346 | 5.997 | 0.000 | 0.000 |
| SYNOV4002883 | 23.940 | 0.000 | 0.000 |
| SYNOV4007430 | 31.677 | 0.000 | 0.000 |
| SYNOV4007671 | 0.000 | 2.649 | 0.000 |
| SYNOV4008440 | 1.475 | 0.000 | 0.000 |
| TESOP2002273 | 0.000 | 14.666 | 0.000 |
| TESOP2002451 | 2.375 | 0.000 | 0.000 |
| TESTI4000014 | 1.964 | 0.000 | 0.000 |
| TESTI4000209 | 2.649 | 0.000 | 0.000 |
| TESTI4001100 | 4.098 | 0.000 | 0.000 |
| TESTI4006137 | 25.755 | 0.000 | 0.000 |
| TESTI4008797 | 12.429 | 0.000 | 0.000 |
| TESTI4009286 | 1.450 | 0.000 | 0.000 |
| TESTI4010851 | 3.280 | 0.000 | 0.000 |
| TESTI4013817 | 27.163 | 0.000 | 0.000 |
| TESTI4014694 | 2.229 | 0.000 | 0.000 |
| TESTI4021478 | 22.098 | 0.000 | 0.000 |
| TESTI4022936 | 26.445 | 0.000 | 0.000 |
| TESTI4024420 | 37.398 | 0.000 | 0.000 |
| TESTI4027821 | 60.471 | 0.000 | 0.000 |
| THYMU2001090 | 21.252 | 0.000 | 0.000 |
| THYMU2033308 | 13.964 | 0.000 | 0.000 |
| THYMU2035735 | 1.319 | 0.000 | 0.000 |
| THYMU2039315 | 54.530 | 0.000 | 0.000 |
| THYMU3001234 | 11.085 | 0.000 | 0.000 |
| THYMU3008171 | 20.170 | 0.000 | 0.000 |
| TKIDN2009641 | 5.782 | 0.000 | 0.000 |
| TKIDN2009889 | 35.077 | 0.000 | 0.000 |
| TKIDN2015788 | 5.261 | 9.505 | 0.000 |
| TRACH1000205 | 19.677 | 0.000 | 0.000 |
| TRACH2001549 | 8.457 | 0.000 | 0.000 |
| TRACH2005811 | 2.109 | 0.000 | 0.000 |
| TRACH2006049 | 47.167 | 0.000 | 0.000 |
| TRACH2007834 | 0.504 | 0.000 | 0.000 |
| TRACH2008300 | 10.186 | 6.135 | 0.000 |
| TRACH2025535 | 5.806 | 0.000 | 0.000 |
| TRACH3001427 | 5.571 | 3.355 | 0.000 |
| TRACH3002192 | 4.989 | 2.253 | 0.000 |
| TRACH3004068 | 0.000 | 5.150 | 0.000 |
| TRACH3004721 | 8.721 | 0.000 | 0.000 |
| TRACH3005294 | 7.428 | 0.000 | 0.000 |
| TRACH3007479 | 1.075 | 0.000 | 0.000 |
| TRACH3008093 | 2.449 | 0.000 | 0.000 |
| TRACH3009455 | 47.167 | 0.000 | 0.000 |
| UTERU2005621 | 0.000 | 8.145 | 0.000 |
| UTERU2006115 | 7.837 | 0.000 | 0.000 |
| UTERU2019706 | 45.606 | 0.000 | 0.000 |
| UTERU2023039 | 45.606 | 0.000 | 0.000 |
| UTERU2026203 | 45.606 | 0.000 | 0.000 |
| UTERU3005230 | 24.419 | 0.000 | 0.000 |
| UTERU3007640 | 45.606 | 0.000 | 0.000 |
| UTERU3009871 | 36.230 | 0.000 | 0.000 |
| ADRGL2000042 | 2.540 | 0.000 | 0.000 |
| BLADE2006830 | 1.681 | 0.000 | 0.000 |
| BRACE2003609 | 9.090 | 0.000 | 0.000 |
| BRALZ2017844 | 0.000 | 49.396 | 0.000 |
| BRAMY3004800 | 38.061 | 0.000 | 0.000 |
| BRAWH1000369 | 100.000 | 0.000 | 0.000 |
| BRAWH2006207 | 12.943 | 0.000 | 0.000 |
| BRAWH2006395 | 12.446 | 0.000 | 0.000 |
| BRAWH2008993 | 49.811 | 0.000 | 0.000 |
| BRAWH2009393 | 100.000 | 0.000 | 0.000 |
| BRAWH2010552 | 58.488 | 0.000 | 0.000 |
| BRAWH3007441 | 100.000 | 0.000 | 0.000 |
| BRAWH3009017 | 100.000 | 0.000 | 0.000 |
| BRHIP2005271 | 7.083 | 0.000 | 0.000 |
| BRHIP3000017 | 8.819 | 0.000 | 0.000 |
| BRHIP3026052 | 0.000 | 54.140 | 0.000 |
| BRTHA2018443 | 22.098 | 0.000 | 0.000 |
| BRTHA3003000 | 17.150 | 0.000 | 0.000 |
| CTONG2020374 | 31.081 | 0.000 | 0.000 |
| CTONG2020378 | 16.140 | 0.000 | 0.000 |
| CTONG2024031 | 2.584 | 0.000 | 0.000 |
| FCBBF1000509 | 3.732 | 0.000 | 0.000 |
| FEBRA2001990 | 18.144 | 0.000 | 0.000 |
| FEBRA2006519 | 11.891 | 0.000 | 0.000 |
| FEBRA2028516 | 8.007 | 0.000 | 0.000 |
| HCHON2000743 | 6.105 | 0.000 | 0.000 |
| IMR322001879 | 9.638 | 0.000 | 0.000 |
| NT2RI2009583 | 0.224 | 0.808 | 0.000 |
| OCBBF2008144 | 5.768 | 0.000 | 0.000 |
| PERIC2007068 | 3.086 | 0.000 | 0.000 |
| PUAEN2006335 | 12.682 | 0.000 | 0.000 |
| SPLEN2039379 | 5.792 | 0.000 | 0.000 |
| TESTI4001984 | 60.471 | 0.000 | 0.000 |
| TESTI4008058 | 8.814 | 0.000 | 0.000 |
| TESTI4025268 | 60.471 | 0.000 | 0.000 |
| TESTI4032090 | 60.471 | 0.000 | 0.000 |
| THYMU3000360 | 39.314 | 0.000 | 0.000 |
| TLIVE2002046 | 5.445 | 0.000 | 0.000 |
| TRACH3000134 | 36.165 | 0.000 | 0.000 |
| UTERU2021820 | 24.929 | 0.000 | 0.000 |
| UTERU2028734 | 21.953 | 0.000 | 0.000 |

The result of comparative analysis of cDNA libraries derived from the substantia nigra (BRSSN), and from whole tissues of a normal brain (BRAWH) showed following genes whose expression levels differed between the two.

TABLE 5

| Clone ID | BRAWH | BRSSN |
|---|---|---|
| ASTRO1000009 | 2.611 | 0.000 |
| BLADE2008398 | 12.401 | 0.000 |
| BRACE1000186 | 4.324 | 0.000 |
| BRACE1000258 | 31.956 | 0.000 |
| BRACE1000533 | 11.795 | 8.780 |
| BRACE2005457 | 58.488 | 0.000 |
| BRACE2010489 | 63.510 | 0.000 |
| BRACE2014657 | 15.451 | 0.000 |
| BRACE2035381 | 10.177 | 0.000 |
| BRACE2044286 | 18.667 | 0.000 |
| BRACE2045954 | 27.309 | 0.000 |
| BRACE3000787 | 32.844 | 0.000 |
| BRACE3003192 | 58.488 | 0.000 |
| BRACE3005499 | 31.276 | 0.000 |
| BRACE3007480 | 19.471 | 0.000 |
| BRACE3009237 | 18.139 | 0.000 |
| BRACE3009724 | 58.488 | 0.000 |
| BRACE3009747 | 2.237 | 8.327 |
| BRACE3010428 | 6.868 | 4.261 |
| BRACE3011271 | 11.036 | 0.000 |
| BRACE3011421 | 28.251 | 0.000 |
| BRACE3012364 | 8.506 | 0.000 |
| BRACE3013780 | 0.000 | 17.852 |
| BRACE3022769 | 4.285 | 5.316 |
| BRACE3026735 | 24.173 | 0.000 |
| BRACE3031838 | 58.488 | 0.000 |
| BRALZ2011796 | 5.511 | 20.514 |
| BRAMY2003008 | 26.445 | 0.000 |
| BRAMY2005052 | 11.612 | 0.000 |
| BRAMY2019300 | 49.811 | 0.000 |
| BRAMY2019963 | 20.428 | 0.000 |
| BRAMY2036567 | 7.474 | 0.000 |
| BRAMY2037823 | 29.664 | 0.000 |
| BRAMY2040592 | 3.482 | 0.000 |
| BRAMY2047420 | 0.000 | 3.770 |
| BRAMY3002803 | 14.428 | 0.000 |
| BRAMY3004224 | 33.027 | 0.000 |
| BRAMY3005091 | 19.193 | 0.000 |
| BRAWH1000127 | 15.983 | 35.693 |
| BRAWH2001395 | 14.290 | 12.514 |
| BRAWH2001671 | 7.605 | 0.000 |
| BRAWH2001940 | 37.398 | 0.000 |
| BRAWH2001973 | 37.398 | 0.000 |
| BRAWH2002560 | 6.454 | 12.010 |
| BRAWH2002761 | 100.000 | 0.000 |
| BRAWH2005315 | 100.000 | 0.000 |
| BRAWH2007658 | 58.101 | 0.000 |
| BRAWH2010000 | 18.745 | 0.000 |
| BRAWH2010084 | 100.000 | 0.000 |
| BRAWH2010536 | 14.718 | 0.000 |
| BRAWH2012162 | 36.060 | 0.000 |
| BRAWH2012326 | 100.000 | 0.000 |
| BRAWH2013294 | 39.442 | 0.000 |
| BRAWH2013871 | 37.485 | 0.000 |
| BRAWH2014414 | 17.865 | 0.000 |
| BRAWH2014645 | 4.228 | 0.000 |
| BRAWH2014662 | 15.521 | 0.000 |
| BRAWH2014876 | 10.473 | 0.000 |
| BRAWH2014954 | 58.488 | 0.000 |
| BRAWH2016221 | 47.417 | 0.000 |
| BRAWH2016439 | 100.000 | 0.000 |
| BRAWH2016702 | 73.807 | 0.000 |
| BRAWH2016724 | 35.119 | 0.000 |
| BRAWH3000078 | 100.000 | 0.000 |
| BRAWH3000100 | 100.000 | 0.000 |
| BRAWH3000314 | 71.553 | 0.000 |
| BRAWH3000491 | 100.000 | 0.000 |
| BRAWH3001326 | 45.606 | 0.000 |
| BRAWH3001475 | 100.000 | 0.000 |
| BRAWH3001891 | 34.539 | 0.000 |
| BRAWH3002574 | 13.222 | 0.000 |
| BRAWH3002600 | 36.800 | 0.000 |
| BRAWH3002819 | 100.000 | 0.000 |
| BRAWH3002821 | 21.953 | 0.000 |
| BRAWH3003522 | 100.000 | 0.000 |
| BRAWH3003555 | 15.229 | 0.000 |
| BRAWH3003727 | 10.055 | 0.000 |
| BRAWH3003801 | 100.000 | 0.000 |
| BRAWH3003992 | 29.008 | 0.000 |
| BRAWH3004453 | 100.000 | 0.000 |
| BRAWH3004666 | 49.499 | 0.000 |
| BRAWH3005132 | 49.811 | 0.000 |
| BRAWH3005422 | 100.000 | 0.000 |
| BRAWH3005912 | 100.000 | 0.000 |
| BRAWH3005981 | 29.324 | 0.000 |
| BRAWH3006548 | 71.018 | 0.000 |
| BRAWH3006792 | 49.499 | 0.000 |
| BRAWH3007221 | 100.000 | 0.000 |
| BRAWH3007506 | 100.000 | 0.000 |
| BRAWH3007592 | 8.966 | 0.000 |
| BRAWH3007726 | 54.530 | 0.000 |
| BRAWH3007783 | 100.000 | 0.000 |
| BRAWH3008341 | 100.000 | 0.000 |
| BRAWH3008697 | 100.000 | 0.000 |
| BRAWH3008931 | 3.463 | 12.891 |
| BRAWH3009297 | 58.488 | 0.000 |
| BRCOC2003213 | 10.381 | 0.000 |
| BRCOC2014033 | 15.633 | 0.000 |
| BRCOC2020142 | 22.014 | 0.000 |
| BRHIP2000920 | 36.630 | 0.000 |
| BRHIP2005719 | 49.499 | 0.000 |
| BRHIP2007741 | 6.943 | 0.000 |
| BRHIP2014228 | 29.439 | 0.000 |
| BRHIP2024146 | 3.042 | 10.190 |
| BRHIP3000339 | 14.290 | 12.514 |
| BRHIP3006683 | 24.100 | 0.000 |
| BRHIP3007586 | 17.255 | 0.000 |
| BRHIP3008405 | 35.187 | 0.000 |
| BRHIP3018797 | 30.810 | 0.000 |
| BRSSN2000684 | 23.433 | 17.444 |
| BRSSN2003086 | 0.000 | 100.000 |
| BRSSN2004496 | 0.000 | 78.696 |
| BRSSN2004719 | 0.000 | 39.002 |
| BRSSN2006892 | 0.000 | 57.631 |
| BRSSN2008549 | 0.000 | 63.611 |
| BRSSN2008797 | 0.000 | 77.045 |
| BRSSN2011262 | 0.000 | 10.489 |
| BRSSN2011738 | 31.553 | 39.146 |
| BRSSN2013874 | 0.000 | 100.000 |
| BRSSN2014299 | 3.695 | 13.753 |
| BRSSN2014424 | 0.000 | 61.866 |
| BRSSN2014556 | 0.000 | 100.000 |
| BRSSN2018581 | 0.000 | 50.137 |
| BRSSN2018925 | 0.000 | 100.000 |
| BRSTN2008052 | 32.844 | 0.000 |
| BRSTN2015015 | 14.017 | 0.000 |
| BRSTN2016470 | 0.438 | 0.000 |
| BRTHA1000311 | 11.803 | 10.982 |
| BRTHA2003461 | 0.000 | 8.169 |
| BRTHA2008335 | 16.281 | 0.000 |
| BRTHA3002427 | 8.577 | 10.641 |
| BRTHA3003490 | 1.623 | 0.000 |
| BRTHA3008520 | 47.417 | 0.000 |
| BRTHA3017848 | 47.417 | 0.000 |
| COLON2001721 | 11.065 | 0.000 |
| CTONG2017500 | 2.649 | 0.000 |
| CTONG2028124 | 0.503 | 0.000 |
| CTONG3000657 | 3.880 | 0.000 |
| CTONG3001123 | 7.847 | 0.000 |
| CTONG3009328 | 11.993 | 0.000 |
| FCBBF2001183 | 16.537 | 6.155 |
| FCBBF3001977 | 7.448 | 0.000 |
| FEBRA2007544 | 14.689 | 0.000 |
| FEBRA2007801 | 4.937 | 0.000 |
| FEBRA2020886 | 12.124 | 0.000 |
| FEBRA2024136 | 0.000 | 42.701 |
| FEBRA2025427 | 0.000 | 9.226 |
| FEBRA2028618 | 5.082 | 0.000 |
| HCASM2007047 | 3.431 | 0.000 |
| HCHON2000244 | 0.658 | 0.000 |
| HCHON2000626 | 2.351 | 4.375 |
| HCHON2001217 | 3.123 | 0.000 |
| HCHON2002676 | 13.647 | 0.000 |
| HCHON2006250 | 1.565 | 0.000 |
| HEART1000074 | 0.759 | 0.000 |
| HHDPC1000118 | 2.096 | 0.000 |
| HLUNG2002465 | 1.209 | 0.000 |
| IMR322000127 | 3.098 | 0.000 |
| IMR322002035 | 36.176 | 0.000 |
| KIDNE2006580 | 7.013 | 0.000 |
| MESAN2006563 | 0.691 | 2.570 |
| MESAN2012054 | 12.754 | 0.000 |
| MESTC1000042 | 2.245 | 0.000 |
| NOVAR2001783 | 4.027 | 0.000 |
| NT2NE2006909 | 0.285 | 0.000 |
| NT2RI2008724 | 1.836 | 0.000 |
| NT2RI2012659 | 13.562 | 0.000 |
| NT2RI2014733 | 24.938 | 0.000 |
| NT2RI3002892 | 8.799 | 0.000 |
| NT2RI3006284 | 4.138 | 0.000 |
| NT2RI3006673 | 19.959 | 0.000 |
| NT2RI3007543 | 1.697 | 0.000 |
| NT2RI3008055 | 44.179 | 0.000 |
| NT2RP7005529 | 14.766 | 0.000 |
| NT2RP7009147 | 14.337 | 0.000 |
| NT2RP7014005 | 5.818 | 0.000 |
| NT2RP7017474 | 18.823 | 0.000 |
| OCBBF2001794 | 4.728 | 0.000 |
| OCBBF2006005 | 9.535 | 0.000 |
| OCBBF2006764 | 15.345 | 0.000 |
| OCBBF2007028 | 9.665 | 0.000 |
| OCBBF2010140 | 32.508 | 0.000 |
| OCBBF2021286 | 18.456 | 0.000 |
| OCBBF2024850 | 4.445 | 0.000 |
| OCBBF2028935 | 5.789 | 8.618 |
| OCBBF2036743 | 11.053 | 0.000 |
| OCBBF2038317 | 19.713 | 0.000 |
| OCBBF3000483 | 11.973 | 0.000 |
| OCBBF3008230 | 29.840 | 0.000 |
| PEBLM2004666 | 3.715 | 0.000 |
| PLACE6001185 | 21.358 | 0.000 |
| PUAEN2005930 | 18.362 | 0.000 |
| PUAEN2006701 | 2.249 | 0.000 |
| PUAEN2007044 | 8.600 | 0.000 |
| PUAEN2009655 | 18.275 | 0.000 |
| SPLEN2028914 | 2.792 | 0.000 |
| SPLEN2031424 | 15.229 | 0.000 |
| SPLEN2031547 | 1.574 | 0.000 |
| SPLEN2034781 | 27.984 | 0.000 |
| SPLEN2036932 | 2.932 | 0.000 |
| SYNOV2014400 | 12.977 | 0.000 |
| SYNOV4002346 | 5.997 | 0.000 |
| SYNOV4002883 | 23.940 | 0.000 |
| SYNOV4007430 | 31.677 | 0.000 |
| SYNOV4008440 | 1.475 | 0.000 |
| TESOP2002451 | 2.375 | 0.000 |
| TESTI4000014 | 1.964 | 0.665 |
| TESTI4000209 | 2.649 | 0.000 |
| TESTI4001100 | 4.098 | 0.000 |
| TESTI4006137 | 25.755 | 0.000 |
| TESTI4008797 | 12.429 | 0.000 |
| TESTI4009286 | 1.450 | 0.000 |
| TESTI4010851 | 3.280 | 2.035 |
| TESTI4013817 | 27.163 | 0.000 |
| TESTI4014694 | 2.229 | 0.000 |
| TESTI4021478 | 22.098 | 0.000 |
| TESTI4022936 | 26.445 | 0.000 |
| TESTI4024420 | 37.398 | 0.000 |
| TESTI4027821 | 60.471 | 0.000 |
| TESTI4037156 | 0.000 | 2.060 |
| THYMU2001090 | 21.252 | 0.000 |
| THYMU2033308 | 13.964 | 0.000 |
| THYMU2035735 | 1.319 | 0.000 |
| THYMU2039315 | 54.530 | 0.000 |
| THYMU3001234 | 11.085 | 0.000 |
| THYMU3008171 | 20.170 | 0.000 |
| TKIDN2009641 | 5.782 | 21.519 |
| TKIDN2009889 | 35.077 | 0.000 |
| TKIDN2015788 | 5.261 | 0.000 |
| TRACH1000205 | 19.677 | 0.000 |
| TRACH2001549 | 8.457 | 0.000 |
| TRACH2005811 | 2.109 | 0.000 |
| TRACH2006049 | 47.167 | 0.000 |
| TRACH2007834 | 0.504 | 1.877 |
| TRACH2008300 | 10.186 | 0.000 |
| TRACH2025535 | 5.806 | 0.000 |
| TRACH3001427 | 5.571 | 0.000 |
| TRACH3002192 | 4.989 | 2.321 |
| TRACH3004721 | 8.721 | 0.000 |
| TRACH3005294 | 7.428 | 0.000 |
| TRACH3007479 | 1.075 | 0.000 |
| TRACH3008093 | 2.449 | 0.000 |
| TRACH3009455 | 47.167 | 0.000 |
| UTERU2006115 | 7.837 | 0.000 |
| UTERU2019706 | 45.606 | 0.000 |
| UTERU2023039 | 45.606 | 0.000 |
| UTERU2026203 | 45.606 | 0.000 |
| UTERU3005230 | 24.419 | 0.000 |
| UTERU3007640 | 45.606 | 0.000 |
| UTERU3009871 | 36.230 | 0.000 |
| ADRGL2000042 | 2.540 | 18.905 |
| BLADE2006830 | 1.681 | 0.000 |
| BRACE2003609 | 9.090 | 0.000 |
| BRAMY3004800 | 38.061 | 0.000 |
| BRAWH1000369 | 100.000 | 0.000 |
| BRAWH2006207 | 12.943 | 48.175 |
| BRAWH2006395 | 12.446 | 0.000 |
| BRAWH2008993 | 49.811 | 0.000 |
| BRAWH2009393 | 100.000 | 0.000 |
| BRAWH2010552 | 58.488 | 0.000 |
| BRAWH3007441 | 100.000 | 0.000 |
| BRAWH3009017 | 100.000 | 0.000 |
| BRHIP2005271 | 7.083 | 0.000 |
| BRHIP3000017 | 8.819 | 0.000 |
| BRTHA2018443 | 22.098 | 0.000 |
| BRTHA3003000 | 17.150 | 63.832 |
| CTONG2020374 | 31.081 | 0.000 |
| CTONG2020378 | 16.140 | 0.000 |
| CTONG2024031 | 2.584 | 0.000 |
| FCBBF1000509 | 3.732 | 6.945 |
| FEBRA2001990 | 18.144 | 0.000 |
| FEBRA2006519 | 11.891 | 0.000 |
| FEBRA2028516 | 8.007 | 0.000 |
| HCHON2000743 | 6.105 | 0.000 |
| IMR322001879 | 9.638 | 0.000 |
| NT2RI2009583 | 0.224 | 1.665 |
| OCBBF2008144 | 5.768 | 0.000 |
| PERIC2007068 | 3.086 | 0.000 |
| PUAEN2006335 | 12.682 | 0.000 |
| SPLEN2039379 | 5.792 | 0.000 |
| TESTI2015626 | 0.000 | 1.224 |
| TESTI4001984 | 60.471 | 0.000 |
| TESTI400805$$ | 8.814 | 0.000 |
| TESTI4025268 | 60.471 | 0.000 |
| TESTI4032090 | 60.471 | 0.000 |
| THYMU3000360 | 39.314 | 0.000 |
| TLIVE2002046 | 5.445 | 0.000 |
| TRACH3000134 | 36.165 | 0.000 |
| UTERU2021820 | 24.929 | 0.000 |
| UTERU2028734 | 21.953 | 0.000 |

The result of comparative analysis of cDNA libraries derived from the hippocampus (BRHIP), and from whole tissues of a normal brain (BRAWH) showed following genes whose expression levels differed between the two.

TABLE 6

| Clone ID | BRAWH | BRHIP |
|---|---|---|
| ASTRO1000009 | 2.611 | 0.000 |
| BLADE2001371 | 0.000 | 12.691 |
| BLADE2008398 | 12.401 | 18.978 |
| BNGH42007788 | 0.000 | 3.141 |
| BRACE1000186 | 4.324 | 2.206 |
| BRACE1000258 | 31.956 | 0.000 |
| BRACE1000533 | 11.795 | 9.627 |
| BRACE2005457 | 58.488 | 0.000 |
| BRACE2010489 | 63.510 | 10.799 |
| BRACE2014657 | 15.451 | 0.000 |
| BRACE2015058 | 0.000 | 8.947 |
| BRACE2018762 | 0.000 | 58.973 |
| BRACE2030341 | 0.000 | 7.057 |
| BRACE2035381 | 10.177 | 20.766 |
| BRACE2044286 | 18.667 | 0.000 |
| BRACE2045954 | 27.309 | 0.000 |
| BRACE3000787 | 32.844 | 0.000 |
| BRACE3003192 | 58.488 | 0.000 |
| BRACE3005499 | 31.276 | 0.000 |
| BRACE3007480 | 19.471 | 52.973 |
| BRACE3009237 | 18.139 | 37.013 |
| BRACE3009724 | 58.488 | 0.000 |
| BRACE3009747 | 2.237 | 2.283 |
| BRACE3010428 | 6.868 | 2.336 |
| BRACE3011271 | 11.036 | 11.259 |
| BRACE3011421 | 28.251 | 0.000 |
| BRACE3012364 | 8.506 | 4.339 |
| BRACE3018963 | 0.000 | 58.973 |
| BRACE3022769 | 4.285 | 4.372 |
| BRACE3026735 | 24.173 | 0.000 |
| BRACE3031838 | 58.488 | 0.000 |
| BRALZ2011796 | 5.511 | 0.000 |
| BRAMY2003008 | 26.445 | 0.000 |
| BRAMY2005052 | 11.612 | 0.000 |
| BRAMY2019300 | 49.811 | 0.000 |
| BRAMY2019963 | 20.428 | 6.947 |
| BRAMY2031317 | 0.000 | 16.004 |
| BRAMY2036567 | 7.474 | 7.626 |
| BRAMY2037823 | 29.664 | 0.000 |
| BRAMY2040592 | 3.482 | 7.105 |
| BRAMY2044078 | 0.000 | 8.947 |
| BRAMY3002803 | 14.428 | 44.161 |
| BRAMY3004224 | 33.027 | 33.695 |
| BRAMY3005091 | 19.193 | 0.000 |
| BRAMY3009811 | 0.000 | 66.943 |
| BRAWH1000127 | 15.983 | 3.261 |
| BRAWH2001395 | 14.290 | 22.297 |
| BRAWH2001671 | 7.605 | 11.638 |
| BRAWH2001940 | 37.398 | 38.155 |
| BRAWH2001973 | 37.398 | 38.155 |
| BRAWH2002560 | 6.454 | 3.292 |
| BRAWH2002761 | 100.000 | 0.000 |
| BRAWH2005315 | 100.000 | 0.000 |
| BRAWH2007658 | 58.101 | 0.000 |
| BRAWH2010000 | 1$$.745 | 3$$.249 |
| BRAWH2010084 | 100.000 | 0.000 |
| BRAWH2010536 | 14.718 | 0.000 |
| BRAWH2012162 | 36.060 | 0.000 |
| BRAWH2012326 | 100.000 | 0.000 |
| BRAWH2013294 | 39.442 | 10.060 |
| BRAWH2013871 | 37.485 | 0.000 |
| BRAWH2014414 | 17.865 | 18.227 |
| BRAWH2014645 | 4.228 | 1.078 |
| BRAWH2014662 | 15.521 | 0.000 |
| BRAWH2014876 | 10.473 | 0.000 |
| BRAWH2014954 | 58.488 | 0.000 |
| BRAWH2016221 | 47.417 | 0.000 |
| BRAWH2016439 | 100.000 | 0.000 |
| BRAWH2016702 | 73.807 | 0.000 |
| BRAWH2016724 | 35.119 | 0.000 |
| BRAWH3000078 | 100.000 | 0.000 |
| BRAWH3000100 | 100.000 | 0.000 |
| BRAWH3000314 | 71.553 | 0.000 |
| BRAWH3000491 | 100.000 | 0.000 |
| BRAWH3001326 | 45.606 | 0.000 |
| BRAWH3001475 | 100.000 | 0.000 |
| BRAWH3001891 | 34.539 | 0.000 |
| BRAWH3002574 | 13.222 | 0.000 |
| BRAWH3002600 | 36.800 | 0.000 |
| BRAWH3002819 | 100.000 | 0.000 |
| BRAWH3002821 | 21.953 | 0.000 |
| BRAWH3003522 | 100.000 | 0.000 |
| BRAWH3003555 | 15.229 | 0.000 |
| BRAWH3003727 | 10.055 | 10.259 |
| BRAWH3003801 | 100.000 | 0.000 |
| BRAWH3003992 | 29.008 | 0.000 |
| BRAWH3004453 | 100.000 | 0.000 |
| BRAWH3004666 | 49.499 | 50.501 |
| BRAWH3005132 | 49.811 | 0.000 |
| BRAWH3005422 | 100.000 | 0.000 |
| BRAWH3005912 | 100.000 | 0.000 |
| BRAWH3005981 | 29.324 | 0.000 |
| BRAWH3006548 | 71.018 | 28.982 |
| BRAWH3006792 | 49.499 | 50.501 |
| BRAWH3007221 | 100.000 | 0.000 |
| BRAWH3007506 | 100.000 | 0.000 |
| BRAWH3007592 | 8.966 | 3.659 |
| BRAWH3007726 | 54.530 | 0.000 |
| BRAWH3007783 | 100.000 | 0.000 |
| BRAWH3008341 | 100.000 | 0.000 |
| BRAWH3008697 | 100.000 | 0.000 |
| BRAWH3008931 | 3.463 | 10.601 |
| BRAWH3009297 | 58.488 | 0.000 |
| BRCAN2020710 | 0.000 | 22.176 |
| BRCAN2028355 | 0.000 | 1.507 |
| BRCOC2003213 | 10.381 | 0.000 |
| BRCOC2014033 | 15.633 | 15.950 |
| BRCOC2020142 | 22.014 | 0.000 |
| BRHIP2000691 | 0.000 | 100.000 |
| BRHIP2000819 | 0.000 | 2.204 |
| BRHIP2000826 | 0.000 | 100.000 |
| BRHIP2000920 | 36.630 | 37.371 |
| BRHIP2001074 | 0.000 | 39.976 |
| BRHIP2001805 | 0.000 | 14.757 |
| BRHIP2001927 | 0.000 | 100.000 |
| BRHIP2002122 | 0.000 | 100.000 |
| BRHIP2002172 | 0.000 | 100.000 |
| BRHIP2002346 | 0.000 | 100.000 |
| BRHIP2003242 | 0.000 | 100.000 |
| BRHIP2003786 | 0.000 | 100.000 |
| BRHIP2003917 | 0.000 | 11.981 |
| BRHIP2004312 | 0.000 | 100.000 |
| BRHIP2004359 | 0.000 | 24.839 |
| BRHIP2004814 | 0.000 | 35.391 |
| BRHIP2004883 | 0.000 | 100.000 |
| BRHIP2005236 | 0.000 | 100.000 |
| BRHIP2005354 | 0.000 | 100.000 |
| BRHIP2005600 | 0.000 | 100.000 |
| BRHIP2005719 | 49.499 | 50.501 |
| BRHIP2005752 | 0.000 | 58.973 |
| BRHIP2005932 | 0.000 | 100.000 |
| BRHIP2006800 | 0.000 | 100.000 |
| BRHIP2007616 | 0.000 | 100.000 |
| BRHIP2007741 | 6.943 | 3.542 |
| BRHIP2009340 | 0.000 | 100.000 |
| BRHIP2009414 | 0.000 | 100.000 |
| BRHIP2009474 | 0.000 | 47.917 |
| BRHIP2013699 | 0.000 | 21.770 |
| BRHIP2014228 | 29.439 | 15.017 |
| BRHIP2021615 | 0.000 | 100.000 |
| BRHIP2022221 | 0.000 | 47.917 |
| BRHIP2024146 | 3.042 | 5.897 |
| BRHIP2024165 | 0.000 | 100.000 |
| BRHIP2026061 | 0.000 | 31.199 |
| BRHIP2026288 | 0.000 | 22.018 |
| BRHIP2029176 | 0.000 | 100.000 |
| BRHIP2029393 | 0.000 | 100.000 |
| BRHIP3000339 | 14.290 | 22.297 |
| BRHIP3000526 | 0.000 | 100.000 |
| BRHIP3001283 | 0.000 | 50.312 |
| BRHIP3006683 | 24.100 | 49.175 |
| BRHIP3007483 | 0.000 | 100.000 |
| BRHIP3007586 | 17.255 | 24.645 |
| BRHIP3008183 | 0.000 | 100.000 |
| BRHIP3008313 | 0.000 | 29.118 |
| BRHIP3008344 | 0.000 | 46.104 |
| BRHIP3008405 | 35.187 | 17.949 |
| BRHIP3008565 | 0.000 | 46.104 |
| BRHIP3008598 | 0.000 | 100.000 |
| BRHIP3008997 | 0.000 | 100.000 |
| BRHIP3009099 | 0.000 | 35.577 |
| BRHIP3009448 | 0.000 | 100.000 |
| BRHIP3011241 | 0.000 | 100.000 |
| BRHIP3013765 | 0.000 | 100.000 |
| BRHIP3013897 | 0.000 | 100.000 |
| BRHIP3015751 | 0.000 | 100.000 |
| BRHIP3016213 | 0.000 | 100.000 |
| BRHIP3018797 | 30.810 | 9.567 |
| BRHIP3020182 | 0.000 | 100.000 |
| BRHIP3024118 | 0.000 | 100.000 |
| BRHIP3024533 | 0.000 | 100.000 |
| BRHIP3024725 | 0.000 | 100.000 |
| BRHIP3025161 | 0.000 | 100.000 |
| BRHIP3025702 | 0.000 | 100.000 |
| BRHIP3026097 | 0.000 | 100.000 |
| BRHIP3027137 | 0.000 | 100.000 |
| BRHIP3027854 | 0.000 | 100.000 |
| BRSSN2000684 | 23.433 | 19.126 |
| BRSSN2004719 | 0.000 | 10.691 |
| BRSSN2008549 | 0.000 | 17.436 |
| BRSSN2011738 | 31.553 | 0.000 |
| BRSSN2014299 | 3.695 | 3.770 |
| BRSTN2008052 | 32.844 | 0.000 |
| BRSTN2015015 | 14.017 | 0.000 |
| BRSTN2016470 | 0.438 | 0.447 |
| BRSTN2018083 | 0.000 | 22.055 |
| BRTHA1000311 | 11.803 | 6.021 |
| BRTHA2002442 | 0.000 | 31.507 |
| BRTHA2008335 | 16.281 | 8.305 |
| BRTHA3000297 | 0.000 | 47.917 |
| BRTHA3001721 | 0.000 | 12.017 |
| BRTHA3002427 | 8.577 | 2.917 |
| BRTHA3003490 | 1.623 | 0.000 |
| BRTHA3005046 | 0.000 | 47.917 |
| BRTHA3008520 | 47.417 | 0.000 |
| BRTHA3008778 | 0.000 | 25.346 |
| BRTHA3009090 | 0.000 | 12.008 |
| BRTHA3015910 | 0.000 | 15.221 |
| BRTHA3017848 | 47.417 | 0.000 |
| COLON2001721 | 11.065 | 5.644 |
| CTONG1000087 | 0.000 | 1.067 |
| CTONG1000088 | 0.000 | 2.981 |
| CTONG1000467 | 0.000 | 8.766 |
| CTONG2000042 | 0.000 | 3.574 |
| CTONG2008233 | 0.000 | 0.701 |
| CTONG2009423 | 0.000 | 35.391 |
| CTONG2017500 | 2.649 | 0.000 |
| CTONG2019788 | 0.000 | 3.120 |
| CTONG2028124 | 0.503 | 1.026 |
| CTONG3000657 | 3.880 | 11.875 |
| CTONG3001123 | 7.847 | 0.000 |
| CTONG3001370 | 0.000 | 3.574 |
| CTONG3002412 | 0.000 | 10.918 |
| CTONG3004072 | 0.000 | 19.915 |
| CTONG3008894 | 0.000 | 2.966 |
| CTONG3009239 | 0.000 | 2.946 |
| CTONG3009328 | 11.993 | 0.000 |
| DFNES2011499 | 0.000 | 6.330 |
| FCBBF2001183 | 16.537 | 11.810 |
| FCBBF3001977 | 7.448 | 7.599 |
| FEBRA2000253 | 0.000 | 14.934 |
| FEBRA2007544 | 14.689 | 4.995 |
| FEBRA2007801 | 4.937 | 7.556 |
| FEBRA2008287 | 0.000 | 3.007 |
| FEBRA2010719 | 0.000 | 17.703 |
| FEBRA2020886 | 12.124 | 12.369 |
| FEBRA2028618 | 5.082 | 0.000 |
| HCASM2007047 | 3.431 | 0.000 |
| HCHON2000028 | 0.000 | 1.619 |
| HCHON2000244 | 0.658 | 0.671 |
| HCHON2000626 | 2.351 | 0.000 |
| HCHON2001217 | 3.123 | 2.124 |
| HCHON2002676 | 13.647 | 0.000 |
| HCHON2005921 | 0.000 | 12.694 |
| HCHON2006250 | 1.565 | 0.000 |
| HEART1000074 | 0.759 | 0.000 |
| HEART2007031 | 0.000 | 11.593 |
| HHDPC1000118 | 2.096 | 6.415 |
| HLUNG2002465 | 1.209 | 3.702 |
| HLUNG2003003 | 0.000 | 16.306 |
| IMR322000127 | 3.098 | 3.161 |
| IMR322001380 | 0.000 | 2.022 |
| IMR322002035 | 36.176 | 0.000 |
| KIDNE2005543 | 0.000 | 18.168 |
| KIDNE2006580 | 7.013 | 14.310 |
| MESAN2006563 | 0.691 | 0.000 |
| MESAN2012054 | 12.754 | 15.615 |
| MESTC1000042 | 2.245 | 0.000 |
| NOVAR2001783 | 4.027 | 0.000 |
| NT2NE2006909 | 0.285 | 0.000 |
| NT2RI2008724 | 1.836 | 1.874 |
| NT2RI2012659 | 13.562 | 0.000 |
| NT2RI2014733 | 24.938 | 0.000 |
| NT2RI2018311 | 0.000 | 21.918 |
| NT2RI3001515 | 0.000 | 11.050 |
| NT2RI3002892 | 8.799 | 16.159 |
| NT2RI3004510 | 0.000 | 28.761 |
| NT2RI3005724 | 0.000 | 12.093 |
| NT2RI3006284 | 4.138 | 0.000 |
| NT2RI3006673 | 19.959 | 10.182 |
| NT2RI3007291 | 0.000 | 12.770 |
| NT2RI3007543 | 1.697 | 0.866 |
| NT2RI3008055 | 44.179 | 0.000 |
| NT2RP7005529 | 14.766 | 15.065 |
| NT2RP7009147 | 14.337 | 1.625 |
| NT2RP7014005 | 5.818 | 0.000 |
| NT2RP7017474 | 18.823 | 0.000 |
| OCBBF2001794 | 4.728 | 0.000 |
| OCBBF2003819 | 0.000 | 21.496 |
| OCBBF2006005 | 9.535 | 9.728 |
| OCBBF2006151 | 0.000 | 11.764 |
| OCBBF2006764 | 15.345 | 0.000 |
| OCBBF2007028 | 9.665 | 11.269 |
| OCBBF2007068 | 0.000 | 45.093 |
| OCBBF2010140 | 32.508 | 0.000 |
| OCBBF2020741 | 0.000 | 28.789 |
| OCBBF2021286 | 18.456 | 18.829 |
| OCBBF2024719 | 0.000 | 30.261 |
| OCBBF2024850 | 4.445 | 4.535 |
| OCBBF2028935 | 5.789 | 7.087 |
| OCBBF2036743 | 11.053 | 0.000 |
| OCBBF2038317 | 19.713 | 0.000 |
| OCBBF3000296 | 0.000 | 19.328 |
| OCBBF3000483 | 11.973 | 8.143 |
| OCBBF3008230 | 29.840 | 0.000 |
| PEBLM2004666 | 3.715 | 0.000 |
| PLACE6001185 | 21.358 | 0.000 |
| PUAEN2005930 | 18.362 | 9.367 |
| PUAEN2006701 | 2.249 | 2.295 |
| PUAEN2007044 | 8.600 | 0.000 |
| PUAEN2009655 | 18.275 | 7.458 |
| SPLEN2010912 | 0.000 | 12.636 |
| SPLEN2012624 | 0.000 | 10.317 |
| SPLEN2028914 | 2.792 | 0.000 |
| SPLEN2031424 | 15.229 | 0.000 |
| SPLEN2031547 | 1.574 | 11.244 |
| SPLEN2034781 | 27.984 | 0.000 |
| SPLEN2036932 | 2.932 | 5.982 |
| SYNOV2014400 | 12.977 | 13.240 |
| SYNOV4002346 | 5.997 | 0.000 |
| SYNOV4002883 | 23.940 | 24.425 |
| SYNOV4007430 | 31.677 | 0.000 |
| SYNOV4008440 | 1.475 | 3.010 |
| TESOP2002451 | 2.375 | 0.000 |
| TESTI2049246 | 0.000 | 53.653 |
| TESTI4000014 | 1.964 | 2.551 |
| TESTI4000209 | 2.649 | 0.000 |
| TESTI4001100 | 4.098 | 4.180 |
| TESTI4006137 | 25.755 | 0.000 |
| TESTI4008797 | 12.429 | 12.681 |
| TESTI4009286 | 1.450 | 1.479 |
| TESTI4010377 | 0.000 | 60.949 |
| TESTI4010851 | 3.280 | 3.904 |
| TESTI4010928 | 0.000 | 34.221 |
| TESTI4011161 | 0.000 | 21.496 |
| TESTI4013817 | 27.163 | 27.712 |
| TESTI4014159 | 0.000 | 23.916 |
| TESTI4014694 | 2.229 | 0.000 |
| TESTI4014818 | 0.000 | 60.949 |
| TESTI4021478 | 22.098 | 0.000 |
| TESTI4022936 | 26.445 | 0.000 |
| TESTI4024420 | 37.398 | 38.155 |
| TESTI4027821 | 60.471 | 0.000 |
| TESTI4037156 | 0.000 | 0.282 |
| THYMU2001090 | 21.252 | 0.000 |
| THYMU2023967 | 0.000 | 31.165 |
| THYMU2025707 | 0.000 | 9.440 |
| THYMU2031341 | 0.000 | 7.040 |
| THYMU2033308 | 13.964 | 0.000 |
| THYMU2035735 | 1.319 | 2.692 |
| THYMU2037226 | 0.000 | 34.964 |
| THYMU2039315 | 54.530 | 0.000 |
| THYMU3001234 | 11.085 | 0.000 |
| THYMU3001379 | 0.000 | 25.695 |
| THYMU3004835 | 0.000 | 20.041 |
| THYMU3007137 | 0.000 | 37.956 |
| THYMU3008171 | 20.170 | 20.579 |
| TKIDN2009641 | 5.782 | 0.000 |
| TKIDN2009889 | 35.077 | 0.000 |
| TKIDN2015788 | 5.261 | 2.684 |
| TRACH1000205 | 19.677 | 4.015 |
| TRACH2001549 | 8.457 | 21.569 |
| TRACH2005811 | 2.109 | 2.152 |
| TRACH2006049 | 47.167 | 0.000 |
| TRACH2007834 | 0.504 | 0.515 |
| TRACH2008300 | 10.186 | 13.857 |
| TRACH2025535 | 5.806 | 2.369 |
| TRACH3000014 | 0.000 | 18.547 |
| TRACH3001427 | 5.571 | 2.842 |
| TRACH3002192 | 4.989 | 6.999 |
| TRACH3004721 | 8.721 | 2.966 |
| TRACH3005294 | 7.428 | 0.000 |
| TRACH3007479 | 1.075 | 0.000 |
| TRACH3008093 | 2.449 | 0.000 |
| TRACH3009455 | 47.167 | 0.000 |
| TUTER1000122 | 0.000 | 3.330 |
| TUTER2000904 | 0.000 | 5.697 |
| UTERU2004929 | 0.000 | 11.843 |
| UTERU2006115 | 7.837 | 0.000 |
| UTERU2019706 | 45.606 | 0.000 |
| UTERU2021163 | 0.000 | 46.104 |
| UTERU2023039 | 45.606 | 0.000 |
| UTERU2026203 | 45.606 | 0.000 |
| UTERU2030213 | 0.000 | 25.133 |
| UTERU3001572 | 0.000 | 1.862 |
| UTERU3003135 | 0.000 | 33.486 |
| UTERU3005230 | 24.419 | 0.000 |
| UTERU3007640 | 45.606 | 0.000 |
| UTERU3009259 | 0.000 | 46.104 |
| UTERU3009871 | 36.230 | 18.482 |
| ADRGL2000042 | 2.540 | 0.000 |
| BLADE2006830 | 1.681 | 0.000 |
| BRACE2003609 | 9.090 | 0.000 |
| BRAMY3004800 | 38.061 | 46.598 |
| BRAWH1000369 | 100.000 | 0.000 |
| BRAWH2006207 | 12.943 | 0.000 |
| BRAWH2006395 | 12.446 | 0.000 |
| BRAWH2008993 | 49.811 | 0.000 |
| BRAWH2009393 | 100.000 | 0.000 |
| BRAWH2010552 | 58.488 | 0.000 |
| BRAWH3007441 | 100.000 | 0.000 |
| BRAWH3009017 | 100.000 | 0.000 |
| BRHIP2002722 | 0.000 | 100.000 |
| BRHIP2003272 | 0.000 | 100.000 |
| BRHIP2005271 | 7.083 | 14.453 |
| BRHIP2005724 | 0.000 | 100.000 |
| BRHIP2006617 | 0.000 | 100.000 |
| BRHIP2008389 | 0.000 | 100.000 |
| BRHIP2012360 | 0.000 | 100.000 |
| BRHIP2017553 | 0.000 | 100.000 |
| BRHIP2026877 | 0.000 | 30.781 |
| BRHIP3000017 | 8.819 | 4.499 |
| BRHIP3000240 | 0.000 | 32.393 |
| BRHIP3008314 | 0.000 | 100.000 |
| BRHIP3026052 | 0.000 | 45.860 |
| BRTHA2018443 | 22.098 | 0.000 |
| BRTHA3003000 | 17.150 | 0.000 |
| CTONG2020374 | 31.081 | 0.000 |
| CTONG2020378 | 16.140 | 16.467 |
| CTONG2024031 | 2.584 | 0.000 |
| CTONG3004726 | 0.000 | 17.278 |
| FCBBF1000509 | 3.732 | 5.711 |
| FEBRA2001990 | 18.144 | 7.404 |
| FEBRA2006519 | 11.891 | 0.000 |
| FEBRA2028516 | 8.007 | 4.668 |
| HCHON2000743 | 6.105 | 0.000 |
| IMR322001879 | 9.638 | 9.833 |
| NT2RI2009583 | 0.224 | 0.228 |
| OCBBF2006987 | 0.000 | 13.187 |
| OCBBF2008144 | 5.768 | 2.942 |
| OCBBF2030116 | 0.000 | 45.093 |
| PERIC2007068 | 3.086 | 4.723 |
| PUAEN2006335 | 12.682 | 0.000 |
| SPLEN2039379 | 5.792 | 8.864 |
| TESTI2015626 | 0.000 | 0.336 |
| TESTI4000214 | 0.000 | 7.979 |
| TESTI4001984 | 60.471 | 0.000 |
| TESTI4008058 | 8.814 | 0.000 |
| TESTI4013894 | 0.000 | 14.289 |
| TESTI4025268 | 60.471 | 0.000 |
| TESTI4025547 | 0.000 | 60.949 |
| TESTI4026207 | 0.000 | 60.949 |
| TESTI4032090 | 60.471 | 0.000 |
| THYMU3000360 | 39.314 | 0.000 |
| TLIVE2002046 | 5.445 | 1.852 |
| TRACH3000134 | 36.165 | 0.000 |
| UTERU2008040 | 0.000 | 24.014 |
| UTERU2021820 | 24.929 | 0.000 |
| UTERU2028734 | 21.953 | 0.000 |

The result of comparative analysis of cDNA libraries derived from the cerebellum (BRACE), and from whole tissues of a normal brain (BRAWH) showed following genes whose expression levels differed between the two.

TABLE 7

| Clone ID | BRAWH | BRACE |
|---|---|---|
| ADRGL2009146 | 0.000 | 10.913 |
| ADRGL2012038 | 0.000 | 1.459 |
| ASTRO1000009 | 2.611 | 0.000 |
| ASTRO2003960 | 0.000 | 17.003 |
| BLADE1000176 | 0.000 | 16.822 |
| BLADE2004089 | 0.000 | 7.034 |
| BLADE2008398 | 12.401 | 4.401 |
| BRACE1000186 | 4.324 | 1.535 |
| BRACE1000258 | 31.956 | 68.044 |
| BRACE1000533 | 11.795 | 11.720 |
| BRACE1000572 | 0.000 | 100.000 |
| BRACE2003639 | 0.000 | 100.000 |
| BRACE2005457 | 58.488 | 41.512 |
| BRACE2006319 | 0.000 | 4.977 |
| BRACE2008594 | 0.000 | 100.000 |
| BRACE2010489 | 63.510 | 15.026 |
| BRACE2011747 | 0.000 | 100.000 |
| BRACE2014306 | 0.000 | 6.909 |
| BRACE2014475 | 0.000 | 100.000 |
| BRACE2014657 | 15.451 | 76.765 |
| BRACE2015058 | 0.000 | 6.224 |
| BRACE2015314 | 0.000 | 100.000 |
| BRACE2016981 | 0.000 | 16.089 |
| BRACE2018762 | 0.000 | 41.027 |
| BRACE2024627 | 0.000 | 8.689 |
| BRACE2026836 | 0.000 | 4.898 |
| BRACE2027258 | 0.000 | 23.981 |
| BRACE2027970 | 0.000 | 45.981 |
| BRACE2028970 | 0.000 | 100.000 |
| BRACE2029112 | 0.000 | 100.000 |
| BRACE2029849 | 0.000 | 100.000 |
| BRACE2030326 | 0.000 | 100.000 |
| BRACE2030341 | 0.000 | 4.909 |
| BRACE2030884 | 0.000 | 100.000 |
| BRACE2031154 | 0.000 | 39.025 |
| BRACE2031389 | 0.000 | 7.220 |
| BRACE2031527 | 0.000 | 100.000 |
| BRACE2031531 | 0.000 | 100.000 |
| BRACE2031899 | 0.000 | 100.000 |
| BRACE2032044 | 0.000 | 100.000 |
| BRACE2032329 | 0.000 | 100.000 |
| BRACE2032385 | 0.000 | 100.000 |
| BRACE2032538 | 0.000 | 100.000 |
| BRACE2032823 | 0.000 | 100.000 |
| BRACE2033720 | 0.000 | 100.000 |
| BRACE2035381 | 10.177 | 36.117 |
| BRACE2035441 | 0.000 | 100.000 |
| BRACE2036005 | 0.000 | 100.000 |
| BRACE2036096 | 0.000 | 100.000 |
| BRACE2036830 | 0.000 | 100.000 |
| BRACE2036834 | 0.000 | 100.000 |
| BRACE2037847 | 0.000 | 100.000 |
| BRACE2038114 | 0.000 | 100.000 |
| BRACE2038329 | 0.000 | 100.000 |
| BRACE2038551 | 0.000 | 100.000 |
| BRACE2039249 | 0.000 | 100.000 |
| BRACE2039327 | 0.000 | 100.000 |
| BRACE2039475 | 0.000 | 100.000 |
| BRACE2039734 | 0.000 | 100.000 |
| BRACE2040138 | 0.000 | 100.000 |
| BRACE2040325 | 0.000 | 100.000 |
| BRACE2041009 | 0.000 | 100.000 |
| BRACE2041200 | 0.000 | 38.787 |
| BRACE2041264 | 0.000 | 100.000 |
| BRACE2042550 | 0.000 | 100.000 |
| BRACE2043142 | 0.000 | 52.057 |
| BRACE2043248 | 0.000 | 100.000 |
| BRACE2043349 | 0.000 | 100.000 |
| BRACE2043665 | 0.000 | 16.915 |
| BRACE2044286 | 18.667 | 26.499 |
| BRACE2044816 | 0.000 | 100.000 |
| BRACE2044949 | 0.000 | 2.766 |
| BRACE2045300 | 0.000 | 100.000 |
| BRACE2045428 | 0.000 | 100.000 |
| BRACE2045596 | 0.000 | 100.000 |
| BRACE2045772 | 0.000 | 2.055 |
| BRACE2045947 | 0.000 | 100.000 |
| BRACE2045954 | 27.309 | 38.766 |
| BRACE2046251 | 0.000 | 100.000 |
| BRACE2046295 | 0.000 | 100.000 |
| BRACE2047011 | 0.000 | 100.000 |
| BRACE2047350 | 0.000 | 45.981 |
| BRACE2047377 | 0.000 | 100.000 |
| BRACE2047385 | 0.000 | 100.000 |
| BRACE3000071 | 0.000 | 9.532 |
| BRACE3000697 | 0.000 | 100.000 |
| BRACE3000787 | 32.844 | 15.541 |
| BRACE3000840 | 0.000 | 100.000 |
| BRACE3000973 | 0.000 | 28.699 |
| BRACE3001002 | 0.000 | 100.000 |
| BRACE3001217 | 0.000 | 100.000 |
| BRACE3001391 | 0.000 | 100.000 |
| BRACE3001595 | 0.000 | 100.000 |
| BRACE3001754 | 0.000 | 38.787 |
| BRACE3002298 | 0.000 | 100.000 |
| BRACE3002390 | 0.000 | 100.000 |
| BRACE3002508 | 0.000 | 100.000 |
| BRACE3003004 | 0.000 | 100.000 |
| BRACE3003192 | 58.488 | 41.512 |
| BRACE3003595 | 0.000 | 100.000 |
| BRACE3003698 | 0.000 | 45.981 |
| BRACE3004058 | 0.000 | 100.000 |
| BRACE3004113 | 0.000 | 100.000 |
| BRACE3004150 | 0.000 | 2.757 |
| BRACE3004358 | 0.000 | 100.000 |
| BRACE3004435 | 0.000 | 100.000 |
| BRACE3004772 | 0.000 | 100.000 |
| BRACE3004783 | 0.000 | 100.000 |
| BRACE3004843 | 0.000 | 22.581 |
| BRACE3004880 | 0.000 | 100.000 |
| BRACE3005145 | 0.000 | 100.000 |
| BRACE3005225 | 0.000 | 100.000 |
| BRACE3005430 | 0.000 | 100.000 |
| BRACE3005499 | 31.276 | 22.199 |
| BRACE3006185 | 0.000 | 100.000 |
| BRACE3006226 | 0.000 | 100.000 |
| BRACE3006462 | 0.000 | 100.000 |
| BRACE3006872 | 0.000 | 100.000 |
| BRACE3007322 | 0.000 | 100.000 |
| BRACE3007472 | 0.000 | 100.000 |
| BRACE3007480 | 19.471 | 13.820 |
| BRACE3007559 | 0.000 | 100.000 |
| BRACE3007625 | 0.000 | 100.000 |
| BRACE3007642 | 0.000 | 100.000 |
| BRACE3007767 | 0.000 | 100.000 |
| BRACE3008036 | 0.000 | 13.098 |
| BRACE3008092 | 0.000 | 100.000 |
| BRACE3008137 | 0.000 | 100.000 |
| BRACE3008384 | 0.000 | 56.141 |
| BRACE3008720 | 0.000 | 100.000 |
| BRACE3008772 | 0.000 | 37.308 |
| BRACE3009090 | 0.000 | 100.000 |
| BRACE3009237 | 18.139 | 12.875 |
| BRACE3009297 | 0.000 | 100.000 |
| BRACE3009377 | 0.000 | 100.000 |
| BRACE3009574 | 0.000 | 100.000 |
| BRACE3009701 | 0.000 | 100.000 |
| BRACE3009708 | 0.000 | 100.000 |
| BRACE3009724 | 58.488 | 41.512 |
| BRACE3009747 | 2.237 | 1.588 |
| BRACE3010397 | 0.000 | 24.243 |
| BRACE3010428 | 6.868 | 3.250 |
| BRACE3011271 | 11.036 | 39.165 |
| BRACE3011421 | 28.251 | 40.103 |
| BRACE3011505 | 0.000 | 100.000 |
| BRACE3012364 | 8.506 | 6.038 |
| BRACE3012930 | 0.000 | 38.787 |
| BRACE3013119 | 0.000 | 100.000 |
| BRACE3013576 | 0.000 | 100.000 |
| BRACE3013740 | 0.000 | 100.000 |
| BRACE3013780 | 0.000 | 3.404 |
| BRACE3014005 | 0.000 | 100.000 |
| BRACE3014068 | 0.000 | 100.000 |
| BRACE3014231 | 0.000 | 100.000 |
| BRACE3014317 | 0.000 | 100.000 |
| BRACE3014807 | 0.000 | 100.000 |
| BRACE3015027 | 0.000 | 9.209 |
| BRACE3015121 | 0.000 | 100.000 |
| BRACE3015262 | 0.000 | 100.000 |
| BRACE3015521 | 0.000 | 100.000 |
| BRACE3015894 | 0.000 | 100.000 |
| BRACE3016884 | 0.000 | 9.165 |
| BRACE3018308 | 0.000 | 100.000 |
| BRACE3018963 | 0.000 | 41.027 |
| BRACE3019055 | 0.000 | 100.000 |
| BRACE3019084 | 0.000 | 100.000 |
| BRACE3020194 | 0.000 | 100.000 |
| BRACE3020286 | 0.000 | 100.000 |
| BRACE3020594 | 0.000 | 100.000 |
| BRACE3022769 | 4.285 | 9.124 |
| BRACE3023912 | 0.000 | 100.000 |
| BRACE3024073 | 0.000 | 100.000 |
| BRACE3024659 | 0.000 | 100.000 |
| BRACE3024662 | 0.000 | 100.000 |
| BRACE3025153 | 0.000 | 100.000 |
| BRACE3025457 | 0.000 | 100.000 |
| BRACE3025531 | 0.000 | 100.000 |
| BRACE3025630 | 0.000 | 100.000 |
| BRACE3026008 | 0.000 | 100.000 |
| BRACE3026075 | 0.000 | 100.000 |
| BRACE3026735 | 24.173 | 51.471 |
| BRACE3027242 | 0.000 | 100.000 |
| BRACE3027326 | 0.000 | 100.000 |
| BRACE3027478 | 0.000 | 11.360 |
| BRACE3030103 | 0.000 | 100.000 |
| BRACE3031838 | 58.488 | 41.512 |
| BRACE3032983 | 0.000 | 100.000 |
| BRACE3040856 | 0.000 | 100.000 |
| BRACE3045033 | 0.000 | 100.000 |
| BRALZ2011796 | 5.511 | 3.912 |
| BRAMY2003008 | 26.445 | 0.000 |
| BRAMY2005052 | 11.612 | 8.242 |
| BRAMY2019300 | 49.811 | 0.000 |
| BRAMY2019963 | 20.428 | 0.000 |
| BRAMY2020058 | 0.000 | 19.826 |
| BRAMY2030098 | 0.000 | 29.935 |
| BRAMY2031317 | 0.000 | 33.400 |
| BRAMY2036567 | 7.474 | 26.525 |
| BRAMY2037823 | 29.664 | 21.055 |
| BRAMY2039872 | 0.000 | 14.071 |
| BRAMY2040592 | 3.482 | 0.000 |
| BRAMY2044078 | 0.000 | 6.224 |
| BRAMY2047420 | 0.000 | 0.719 |
| BRAMY3002620 | 0.000 | 41.329 |
| BRAMY3002803 | 14.428 | 0.000 |
| BRAMY3004224 | 33.027 | 0.000 |
| BRAMY3005091 | 19.193 | 13.622 |
| BRAMY3005932 | 0.000 | 41.329 |
| BRAMY4000229 | 0.000 | 25.113 |
| BRAWH1000127 | 15.983 | 4.538 |
| BRAWH2001395 | 14.290 | 3.580 |
| BRAWH2001671 | 7.605 | 8.097 |
| BRAWH2001940 | 37.398 | 0.000 |
| BRAWH2001973 | 37.398 | 0.000 |
| BRAWH2002560 | 6.454 | 0.000 |
| BRAWH2002761 | 100.000 | 0.000 |
| BRAWH2005315 | 100.000 | 0.000 |
| BRAWH2007658 | 58.101 | 0.000 |
| BRAWH2010000 | 18.745 | 0.000 |
| BRAWH2010084 | 100.000 | 0.000 |
| BRAWH2010536 | 14.718 | 0.000 |
| BRAWH2012162 | 36.060 | 0.000 |
| BRAWH2012326 | 100.000 | 0.000 |
| BRAWH2013294 | 39.442 | 13.997 |
| BRAWH2013871 | 37.485 | 0.000 |
| BRAWH2014414 | 17.865 | 0.000 |
| BRAWH2014645 | 4.228 | 3.751 |
| BRAWH2014662 | 15.521 | 11.016 |
| BRAWH2014876 | 10.473 | 7.433 |
| BRAWH2014954 | 58.488 | 41.512 |
| BRAWH2016221 | 47.417 | 0.000 |
| BRAWH2016439 | 100.000 | 0.000 |
| BRAWH2016702 | 73.807 | 26.193 |
| BRAWH2016724 | 35.119 | 0.000 |
| BRAWH3000078 | 100.000 | 0.000 |
| BRAWH3000100 | 100.000 | 0.000 |
| BRAWH3000314 | 71.553 | 0.000 |
| BRAWH3000491 | 100.000 | 0.000 |
| BRAWH3001326 | 45.606 | 0.000 |
| BRAWH3001475 | 100.000 | 0.000 |
| BRAWH3001891 | 34.539 | 36.772 |
| BRAWH3002574 | 13.222 | 0.000 |
| BRAWH3002600 | 36.800 | 26.120 |
| BRAWH3002819 | 100.000 | 0.000 |
| BRAWH3002821 | 21.953 | 0.000 |
| BRAWH3003522 | 100.000 | 0.000 |
| BRAWH3003555 | 15.229 | 5.405 |
| BRAWH3003727 | 10.055 | 21.411 |
| BRAWH3003801 | 100.000 | 0.000 |
| BRAWH3003992 | 29.008 | 0.000 |
| BRAWH3004453 | 100.000 | 0.000 |
| BRAWH3004666 | 49.499 | 0.000 |
| BRAWH3005132 | 49.811 | 0.000 |
| BRAWH3005422 | 100.000 | 0.000 |
| BRAWH3005912 | 100.000 | 0.000 |
| BRAWH3005981 | 29.324 | 0.000 |
| BRAWH3006548 | 71.018 | 0.000 |
| BRAWH3006792 | 49.499 | 0.000 |
| BRAWH3007221 | 100.000 | 0.000 |
| BRAWH3007506 | 100.000 | 0.000 |
| BRAWH3007592 | 8.966 | 6.364 |
| BRAWH3007726 | 54.530 | 0.000 |
| BRAWH3007783 | 100.000 | 0.000 |
| BRAWH3008341 | 100.000 | 0.000 |
| BRAWH3008697 | 100.000 | 0.000 |
| BRAWH3008931 | 3.463 | 4.916 |
| BRAWH3009297 | 58.488 | 41.512 |
| BRCAN2009432 | 0.000 | 1.869 |
| BRCAN2010376 | 0.000 | 23.627 |
| BRCAN2015371 | 0.000 | 15.560 |
| BRCAN2020710 | 0.000 | 7.714 |
| BRCOC2003213 | 10.381 | 7.368 |
| BRCOC2007034 | 0.000 | 16.691 |
| BRCOC2014033 | 15.633 | 0.000 |
| BRCOC2020142 | 22.014 | 0.000 |
| BRHIP2000920 | 36.630 | 25.999 |
| BRHIP2004359 | 0.000 | 34.560 |
| BRHIP2005719 | 49.499 | 0.000 |
| BRHIP2005752 | 0.000 | 41.027 |
| BRHIP2007741 | 6.943 | 3.696 |
| BRHIP2013699 | 0.000 | 15.145 |
| BRHIP2014228 | 29.439 | 10.447 |
| BRHIP2024146 | 3.042 | 0.864 |
| BRHIP3000339 | 14.290 | 3.580 |
| BRHIP3006683 | 24.100 | 0.000 |
| BRHIP3007586 | 17.255 | 7.348 |
| BRHIP3008313 | 0.000 | 20.257 |
| BRHIP3008405 | 35.187 | 12.487 |
| BRHIP3018797 | 30.810 | 3.803 |
| BRSSN2000684 | 23.433 | 0.000 |
| BRSSN2006892 | 0.000 | 10.990 |
| BRSSN2011262 | 0.000 | 2.000 |
| BRSSN2011738 | 31.553 | 7.465 |
| BRSSN2014299 | 3.695 | 2.623 |
| BRSTN2008052 | 32.844 | 15.541 |
| BRSTN2010750 | 0.000 | 7.220 |
| BRSTN2015015 | 14.017 | 19.897 |
| BRSTN2016470 | 0.438 | 7.146 |
| BRTHA1000311 | 11.803 | 10.472 |
| BRTHA2008335 | 16.281 | 0.000 |
| BRTHA2008955 | 0.000 | 39.025 |
| BRTHA2011194 | 0.000 | 39.025 |
| BRTHA3001721 | 0.000 | 8.360 |
| BRTHA3002427 | 8.577 | 2.029 |
| BRTHA3003490 | 1.623 | 2.304 |
| BRTHA3008520 | 47.417 | 0.000 |
| BRTHA3009090 | 0.000 | 8.354 |
| BRTHA3017848 | 47.417 | 0.000 |
| COLON2001721 | 11.065 | 19.633 |
| CTONG2008233 | 0.000 | 3.412 |
| CTONG2017500 | 2.649 | 0.000 |
| CTONG2028124 | 0.503 | 2.854 |
| CTONG3000657 | 3.880 | 5.508 |
| CTONG3001123 | 7.847 | 0.000 |
| CTONG3005813 | 0.000 | 27.592 |
| CTONG3008894 | 0.000 | 1.032 |
| CTONG3009328 | 11.993 | 0.000 |
| DFNES2011499 | 0.000 | 4.403 |
| FCBBF2001183 | 16.537 | 5.869 |
| FCBBF3001977 | 7.448 | 10.573 |
| FEBRA2006396 | 0.000 | 5.705 |
| FEBRA2007544 | 14.689 | 41.703 |
| FEBRA2007708 | 0.000 | 6.066 |
| FEBRA2007801 | 4.937 | 1.752 |
| FEBRA2008287 | 0.000 | 4.184 |
| FEBRA2020886 | 12.124 | 0.000 |
| FEBRA2021966 | 0.000 | 3.062 |
| FEBRA2026984 | 0.000 | 12.446 |
| FEBRA2028618 | 5.082 | 10.821 |
| HCASM2007047 | 3.431 | 0.000 |
| HCHON2000244 | 0.658 | 0.934 |
| HCHON2000626 | 2.351 | 0.834 |
| HCHON2001217 | 3.123 | 2.216 |
| HCHON2002676 | 13.647 | 0.000 |
| HCHON2005921 | 0.000 | 2.944 |
| HCHON2006250 | 1.565 | 0.000 |
| HEART1000074 | 0.759 | 1.078 |
| HHDPC1000118 | 2.096 | 0.000 |
| HLUNG2002465 | 1.209 | 0.000 |
| IMR322000127 | 3.098 | 4.398 |
| IMR322001380 | 0.000 | 2.814 |
| IMR322002035 | 36.176 | 0.000 |
| KIDNE2000665 | 0.000 | 1.374 |
| KIDNE2006580 | 7.013 | 14.933 |
| MESAN2006563 | 0.691 | 1.470 |
| MESAN2012054 | 12.754 | 5.432 |
| MESTC1000042 | 2.245 | 0.797 |
| NB9N41000340 | 0.000 | 2.059 |
| NESOP2001752 | 0.000 | 3.228 |
| NOVAR2001783 | 4.027 | 0.000 |
| NT2NE2006909 | 0.285 | 0.000 |
| NT2RI2005166 | 0.000 | 18.511 |
| NT2RI2008724 | 1.836 | 0.000 |
| NT2RI2012659 | 13.562 | 48.128 |
| NT2RI2014733 | 24.938 | 0.000 |
| NT2RI2019751 | 0.000 | 28.087 |
| NT2RI3002892 | 8.799 | 9.992 |
| NT2RI3003382 | 0.000 | 28.087 |
| NT2RI3004510 | 0.000 | 20.009 |
| NT2RI3005724 | 0.000 | 42.065 |
| NT2RI3006284 | 4.138 | 1.469 |
| NT2RI3006673 | 19.959 | 0.000 |
| NT2RI3007291 | 0.000 | 35.535 |
| NT2RI3007543 | 1.697 | 1.204 |
| NT2RI3008055 | 44.179 | 15.678 |
| NT2RP7004123 | 0.000 | 1.362 |
| NT2RP7005529 | 14.766 | 5.240 |
| NT2RP7009147 | 14.337 | 0.000 |
| NT2RP7010599 | 0.000 | 22.750 |
| NT2RP7014005 | 5.818 | 0.000 |
| NT2RP7017474 | 18.823 | 13.360 |
| NTONG2005969 | 0.000 | 4.560 |
| OCBBF2001794 | 4.728 | 0.000 |
| OCBBF2003819 | 0.000 | 14.955 |
| OCBBF2006005 | 9.535 | 0.000 |
| OCBBF2006151 | 0.000 | 4.092 |
| OCBBF2006764 | 15.345 | 21.782 |
| OCBBF2007028 | 9.665 | 8.820 |
| OCBBF2010140 | 32.508 | 0.000 |
| OCBBF2020343 | 0.000 | 36.360 |
| OCBBF2020741 | 0.000 | 20.028 |
| OCBBF2021286 | 18.456 | 6.550 |
| OCBBF2022351 | 0.000 | 36.360 |
| OCBBF2024850 | 4.445 | 1.577 |
| OCBBF2025527 | 0.000 | 36.360 |
| OCBBF2028935 | 5.789 | 1.643 |
| OCBBF2036743 | 11.053 | 15.690 |
| OCBBF2038317 | 19.713 | 0.000 |
| OCBBF3000483 | 11.973 | 14.163 |
| OCBBF3007516 | 0.000 | 2.684 |
| OCBBF3008230 | 29.840 | 0.000 |
| PEBLM2004666 | 3.715 | 0.000 |
| PERIC2000889 | 0.000 | 6.314 |
| PLACE6001185 | 21.358 | 15.159 |
| PUAEN2002489 | 0.000 | 9.262 |
| PUAEN2005930 | 18.362 | 0.000 |
| PUAEN2006701 | 2.249 | 0.000 |
| PUAEN2007044 | 8.600 | 5.087 |
| PUAEN2009655 | 18.275 | 0.000 |
| SPLEN2010912 | 0.000 | 26.371 |
| SPLEN2012624 | 0.000 | 7.178 |
| SPLEN2027268 | 0.000 | 14.361 |
| SPLEN2028914 | 2.792 | 0.000 |
| SPLEN2031424 | 15.229 | 5.405 |
| SPLEN2031547 | 1.574 | 5.587 |
| SPLEN2034781 | 27.984 | 0.000 |
| SPLEN2036932 | 2.932 | 2.081 |
| SPLEN2037194 | 0.000 | 28.977 |
| SYNOV2014400 | 12.977 | 0.000 |
| SYNOV4002346 | 5.997 | 0.000 |
| SYNOV4002883 | 23.940 | 0.000 |
| SYNOV4007430 | 31.677 | 0.000 |
| SYNOV4007671 | 0.000 | 0.520 |
| SYNOV4008440 | 1.475 | 2.094 |
| TESOP2002273 | 0.000 | 2.881 |
| TESOP2002451 | 2.375 | 3.372 |
| TESOP2002950 | 0.000 | 9.399 |
| TESTI1000330 | 0.000 | 52.057 |
| TESTI4000014 | 1.964 | 2.028 |
| TESTI4000209 | 2.649 | 0.000 |
| TESTI4000349 | 0.000 | 36.899 |
| TESTI4001100 | 4.098 | 8.725 |
| TESTI4001561 | 0.000 | 28.578 |
| TESTI4006137 | 25.755 | 0.000 |
| TESTI4008797 | 12.429 | 0.000 |
| TESTI4009286 | 1.450 | 0.000 |
| TESTI4010851 | 3.280 | 2.716 |
| TESTI4011161 | 0.000 | 14.955 |
| TESTI4013675 | 0.000 | 14.015 |
| TESTI4013817 | 27.163 | 0.000 |
| TESTI4014159 | 0.000 | 16.638 |
| TESTI4014306 | 0.000 | 52.057 |
| TESTI4014694 | 2.229 | 0.791 |
| TESTI4021478 | 22.098 | 0.000 |
| TESTI4022936 | 26.445 | 0.000 |
| TESTI4024420 | 37.398 | 0.000 |
| TESTI4027821 | 60.471 | 0.000 |
| TESTI4037156 | 0.000 | 0.982 |
| TESTI4046819 | 0.000 | 14.013 |
| THYMU2001090 | 21.252 | 0.000 |
| THYMU2016523 | 0.000 | 23.416 |
| THYMU2023967 | 0.000 | 43.363 |
| THYMU2030264 | 0.000 | 45.981 |
| THYMU2033308 | 13.964 | 0.000 |
| THYMU2035735 | 1.319 | 0.936 |
| THYMU2039315 | 54.530 | 0.000 |
| THYMU2039780 | 0.000 | 45.981 |
| THYMU3001083 | 0.000 | 45.981 |
| THYMU3001234 | 11.085 | 7.868 |
| THYMU3003309 | 0.000 | 20.635 |
| THYMU3006485 | 0.000 | 45.981 |
| THYMU3008171 | 20.170 | 14.316 |
| TKIDN2009641 | 5.782 | 4.104 |
| TKIDN2009889 | 35.077 | 0.000 |
| TKIDN2015788 | 5.261 | 0.000 |
| TRACH1000205 | 19.677 | 8.380 |
| TRACH2001549 | 8.457 | 0.000 |
| TRACH2005811 | 2.109 | 0.000 |
| TRACH2006049 | 47.167 | 0.000 |
| TRACH2007834 | 0.504 | 0.000 |
| TRACH2008300 | 10.186 | 3.615 |
| TRACH2025535 | 5.806 | 2.472 |
| TRACH3001427 | 5.571 | 3.295 |
| TRACH3002192 | 4.989 | 3.541 |
| TRACH3004721 | 8.721 | 4.127 |
| TRACH3005294 | 7.428 | 0.000 |
| TRACH3006038 | 0.000 | 38.787 |
| TRACH3006412 | 0.000 | 38.787 |
| TRACH3007479 | 1.075 | 0.000 |
| TRACH3008093 | 2.449 | 3.477 |
| TRACH3009455 | 47.167 | 0.000 |
| TUTER2000904 | 0.000 | 5.944 |
| UTERU2002410 | 0.000 | 0.417 |
| UTERU2006115 | 7.837 | 5.563 |
| UTERU2007520 | 0.000 | 4.761 |
| UTERU2019706 | 45.606 | 0.000 |
| UTERU2023039 | 45.606 | 0.000 |
| UTERU2026203 | 45.606 | 0.000 |
| UTERU3000226 | 0.000 | 37.308 |
| UTERU3001572 | 0.000 | 2.591 |
| UTERU3005230 | 24.419 | 17.332 |
| UTERU3005460 | 0.000 | 37.308 |
| UTERU3005970 | 0.000 | 37.308 |
| UTERU3006308 | 0.000 | 37.308 |
| UTERU3007419 | 0.000 | 37.308 |
| UTERU3007640 | 45.606 | 0.000 |
| UTERU3007913 | 0.000 | 12.951 |
| UTERU3009871 | 36.230 | 0.000 |
| ADRGL2000042 | 2.540 | 7.210 |
| BLADE2006830 | 1.681 | 2.386 |
| BRACE2002589 | 0.000 | 100.000 |
| BRACE2003609 | 9.090 | 6.452 |
| BRACE2009318 | 0.000 | 100.000 |
| BRACE2011677 | 0.000 | 100.000 |
| BRACE2029396 | 0.000 | 100.000 |
| BRACE2037299 | 0.000 | 100.000 |
| BRACE2039823 | 0.000 | 41.329 |
| BRACE2039832 | 0.000 | 100.000 |
| BRACE2043105 | 0.000 | 100.000 |
| BRACE3001058 | 0.000 | 100.000 |
| BRACE3001113 | 0.000 | 18.680 |
| BRACE3003026 | 0.000 | 11.590 |
| BRACE3003053 | 0.000 | 36.360 |
| BRACE3009127 | 0.000 | 100.000 |
| BRACE3010076 | 0.000 | 100.000 |
| BRACE3015829 | 0.000 | 100.000 |
| BRACE3021148 | 0.000 | 100.000 |
| BRAMY3004800 | 38.061 | 0.000 |
| BRAWH1000369 | 100.000 | 0.000 |
| BRAWH2006207 | 12.943 | 9.187 |
| BRAWH2006395 | 12.446 | 0.000 |
| BRAWH2008993 | 49.811 | 0.000 |
| BRAWH2009393 | 100.000 | 0.000 |
| BRAWH2010552 | 58.488 | 41.512 |
| BRAWH3007441 | 100.000 | 0.000 |
| BRAWH3009017 | 100.000 | 0.000 |
| BRCOC2019841 | 0.000 | 16.691 |
| BRHIP2005271 | 7.083 | 0.000 |
| BRHIP3000017 | 8.819 | 0.000 |
| BRHIP3000240 | 0.000 | 67.607 |
| BRTHA2018443 | 22.098 | 0.000 |
| BRTHA3003000 | 17.150 | 0.000 |
| CTONG2020374 | 31.081 | 11.030 |
| CTONG2020378 | 16.140 | 0.000 |
| CTONG2024031 | 2.584 | 3.669 |
| FCBBF1000509 | 3.732 | 0.000 |
| FEBRA2001990 | 18.144 | 15.453 |
| FEBRA2006519 | 11.891 | 25.320 |
| FEBRA2028516 | 8.007 | 1.624 |
| HCHON2000743 | 6.105 | 8.667 |
| IMR322001879 | 9.638 | 0.000 |
| NT2RI2009583 | 0.224 | 5.399 |
| NT2RP8000521 | 0.000 | 37.067 |
| OCBBF2008144 | 5.768 | 4.094 |
| OCBBF2011669 | 0.000 | 36.360 |
| PERIC2007068 | 3.086 | 8.761 |
| PUAEN2006335 | 12.682 | 0.000 |
| SPLEN2039379 | 5.792 | 10.278 |
| SYNOV2021953 | 0.000 | 6.793 |
| TESTI2015626 | 0.000 | 0.467 |
| TESTI4001984 | 60.471 | 0.000 |
| TESTI4008058 | 8.814 | 0.000 |
| TESTI4013894 | 0.000 | 19.881 |
| TESTI4025268 | 60.471 | 0.000 |
| TESTI4032090 | 60.471 | 0.000 |
| THYMU2004284 | 0.000 | 4.768 |
| THYMU2040925 | 0.000 | 45.981 |
| THYMU3000360 | 39.314 | 27.904 |
| TLIVE2002046 | 5.445 | 7.729 |
| TRACH3000134 | 36.165 | 0.000 |
| UTERU2008040 | 0.000 | 8.353 |
| UTERU2011220 | 0.000 | 1.389 |
| UTERU2021820 | 24.929 | 17.694 |
| UTERU2028734 | 21.953 | 0.000 |

The result of comparative analysis of cDNA libraries derived from the thalamus (BRTHA), and from whole tissues of a normal brain (BRAWH) showed following genes whose expression levels differed between the two.

TABLE 8

| Clone ID | BRAWH | BRTHA |
|---|---|---|
| ASTRO1000009 | 2.611 | 0.000 |
| ASTRO3000482 | 0.000 | 24.247 |
| BLADE2008398 | 12.401 | 13.752 |
| BRACE1000186 | 4.324 | 0.000 |
| BRACE1000258 | 31.956 | 0.000 |
| BRACE1000533 | 11.795 | 5.232 |
| BRACE2005457 | 58.488 | 0.000 |
| BRACE2010489 | 63.510 | 0.000 |
| BRACE2014306 | 0.000 | 5.398 |
| BRACE2014657 | 15.451 | 0.000 |
| BRACE2015058 | 0.000 | 19.449 |
| BRACE2031154 | 0.000 | 60.975 |
| BRACE2035381 | 10.177 | 11.286 |
| BRACE2044286 | 18.667 | 0.000 |
| BRACE2045954 | 27.309 | 0.000 |
| BRACE3000787 | 32.844 | 12.141 |
| BRACE3003192 | 58.488 | 0.000 |
| BRACE3005499 | 31.276 | 0.000 |
| BRACE3007480 | 19.471 | 7.197 |
| BRACE3008384 | 0.000 | 43.859 |
| BRACE3009237 | 18.139 | 20.116 |
| BRACE3009724 | 58.488 | 0.000 |
| BRACE3009747 | 2.237 | 2.481 |
| BRACE3010397 | 0.000 | 75.757 |
| BRACE3010428 | 6.868 | 2.539 |
| BRACE3011271 | 11.036 | 0.000 |
| BRACE3011421 | 28.251 | 0.000 |
| BRACE3012364 | 8.506 | 9.433 |
| BRACE3022769 | 4.285 | 4.752 |
| BRACE3026735 | 24.173 | 0.000 |
| BRACE3027478 | 0.000 | 35.497 |
| BRACE3031838 | 58.488 | 0.000 |
| BRALZ2011796 | 5.511 | 6.112 |
| BRAMY2003008 | 26.445 | 0.000 |
| BRAMY2005052 | 11.612 | 25.755 |
| BRAMY2019300 | 49.811 | 0.000 |
| BRAMY2019963 | 20.428 | 7.551 |
| BRAMY2028914 | 0.000 | 19.645 |
| BRAMY2031317 | 0.000 | 34.791 |
| BRAMY2036567 | 7.474 | 0.000 |
| BRAMY2037823 | 29.664 | 0.000 |
| BRAMY2040592 | 3.482 | 0.000 |
| BRAMY2044078 | 0.000 | 19.449 |
| BRAMY3002803 | 14.428 | 0.000 |
| BRAMY3004224 | 33.027 | 0.000 |
| BRAMY3005091 | 19.193 | 0.000 |
| BRAMY4000229 | 0.000 | 39.237 |
| BRAWH1000127 | 15.983 | 7.090 |
| BRAWH2001395 | 14.290 | 11.652 |
| BRAWH2001671 | 7.605 | 12.650 |
| BRAWH2001940 | 37.398 | 0.000 |
| BRAWH2001973 | 37.398 | 0.000 |
| BRAWH2002560 | 6.454 | 0.000 |
| BRAWH2002761 | 100.000 | 0.000 |
| BRAWH2005315 | 100.000 | 0.000 |
| BRAWH2007658 | 58.101 | 0.000 |
| BRAWH2010000 | 18.745 | 0.000 |
| BRAWH2010084 | 100.000 | 0.000 |
| BRAWH2010536 | 14.718 | 0.000 |
| BRAWH2012162 | 36.060 | 0.000 |
| BRAWH2012326 | 100.000 | 0.000 |
| BRAWH2013294 | 39.442 | 0.000 |
| BRAWH2013871 | 37.485 | 0.000 |
| BRAWH2014414 | 17.865 | 9.906 |
| BRAWH2014645 | 4.228 | 1.172 |
| BRAWH2014662 | 15.521 | 17.212 |
| BRAWH2014876 | 10.473 | 58.068 |
| BRAWH2014954 | 58.488 | 0.000 |
| BRAWH2016221 | 47.417 | 52.583 |
| BRAWH2016439 | 100.000 | 0.000 |
| BRAWH2016702 | 73.807 | 0.000 |
| BRAWH2016724 | 35.119 | 0.000 |
| BRAWH3000078 | 100.000 | 0.000 |
| BRAWH3000100 | 100.000 | 0.000 |
| BRAWH3000314 | 71.553 | 0.000 |
| BRAWH3000491 | 100.000 | 0.000 |
| BRAWH3001326 | 45.606 | 0.000 |
| BRAWH3001475 | 100.000 | 0.000 |
| BRAWH3001891 | 34.539 | 0.000 |
| BRAWH3002574 | 13.222 | 0.000 |
| BRAWH3002600 | 36.800 | 0.000 |
| BRAWH3002819 | 100.000 | 0.000 |
| BRAWH3002821 | 21.953 | 0.000 |
| BRAWH3003522 | 100.000 | 0.000 |
| BRAWH3003555 | 15.229 | 8.444 |
| BRAWH3003727 | 10.055 | 0.000 |
| BRAWH3003801 | 100.000 | 0.000 |
| BRAWH3003992 | 29.008 | 0.000 |
| BRAWH3004453 | 100.000 | 0.000 |
| BRAWH3004666 | 49.499 | 0.000 |
| BRAWH3005132 | 49.811 | 0.000 |
| BRAWH3005422 | 100.000 | 0.000 |
| BRAWH3005912 | 100.000 | 0.000 |
| BRAWH3005981 | 29.324 | 0.000 |
| BRAWH3006548 | 71.018 | 0.000 |
| BRAWH3006792 | 49.499 | 0.000 |
| BRAWH3007221 | 100.000 | 0.000 |
| BRAWH3007506 | 100.000 | 0.000 |
| BRAWH3007592 | 8.966 | 0.000 |
| BRAWH3007726 | 54.530 | 0.000 |
| BRAWH3007783 | 100.000 | 0.000 |
| BRAWH3008341 | 100.000 | 0.000 |
| BRAWH3008697 | 100.000 | 0.000 |
| BRAWH3008931 | 3.463 | 0.000 |
| BRAWH3009297 | 58.488 | 0.000 |
| BRCAN2006297 | 0.000 | 15.966 |
| BRCOC2003213 | 10.381 | 0.000 |
| BRCOC2014033 | 15.633 | 0.000 |
| BRCOC2020142 | 22.014 | 0.000 |
| BRHIP2000819 | 0.000 | 4.791 |
| BRHIP2000920 | 36.630 | 0.000 |
| BRHIP2005719 | 49.499 | 0.000 |
| BRHIP2007741 | 6.943 | 5.775 |
| BRHIP2009474 | 0.000 | 52.083 |
| BRHIP2013699 | 0.000 | 23.663 |
| BRHIP2014228 | 29.439 | 0.000 |
| BRHIP2022221 | 0.000 | 52.083 |
| BRHIP2024146 | 3.042 | 7.759 |
| BRHIP3000339 | 14.290 | 11.652 |
| BRHIP3006683 | 24.100 | 26.726 |
| BRHIP3007586 | 17.255 | 11.481 |
| BRHIP3008405 | 35.187 | 0.000 |
| BRHIP3018797 | 30.810 | 10.399 |
| BRSSN2000684 | 23.433 | 10.395 |
| BRSSN2008549 | 0.000 | 18.953 |
| BRSSN2008797 | 0.000 | 22.955 |
| BRSSN2011738 | 31.553 | 0.000 |
| BRSSN2014299 | 3.695 | 0.000 |
| BRSTN2004863 | 0.000 | 11.049 |
| BRSTN2008052 | 32.844 | 12.141 |
| BRSTN2015015 | 14.017 | 15.544 |
| BRSTN2016470 | 0.438 | 0.000 |
| BRTHA1000311 | 11.803 | 9.817 |
| BRTHA2000855 | 0.000 | 100.000 |
| BRTHA2001462 | 0.000 | 100.000 |
| BRTHA2002115 | 0.000 | 100.000 |
| BRTHA2002281 | 0.000 | 100.000 |
| BRTHA2002376 | 0.000 | 100.000 |
| BRTHA2002442 | 0.000 | 68.493 |
| BRTHA2002493 | 0.000 | 100.000 |
| BRTHA2002608 | 0.000 | 100.000 |
| BRTHA2002808 | 0.000 | 12.857 |
| BRTHA2003030 | 0.000 | 100.000 |
| BRTHA2003110 | 0.000 | 100.000 |
| BRTHA2003116 | 0.000 | 100.000 |
| BRTHA2003461 | 0.000 | 2.434 |
| BRTHA2004821 | 0.000 | 100.000 |
| BRTHA2004978 | 0.000 | 100.000 |
| BRTHA2005579 | 0.000 | 100.000 |
| BRTHA2005956 | 0.000 | 100.000 |
| BRTHA2006075 | 0.000 | 100.000 |
| BRTHA2006146 | 0.000 | 100.000 |
| BRTHA2006194 | 0.000 | 100.000 |
| BRTHA2007122 | 0.000 | 100.000 |
| BRTHA2007422 | 0.000 | 100.000 |
| BRTHA2007603 | 0.000 | 100.000 |
| BRTHA2008316 | 0.000 | 100.000 |
| BRTHA2008335 | 16.281 | 9.028 |
| BRTHA2008527 | 0.000 | 100.000 |
| BRTHA2008535 | 0.000 | 100.000 |
| BRTHA2008955 | 0.000 | 60.975 |
| BRTHA2009311 | 0.000 | 100.000 |
| BRTHA2009846 | 0.000 | 100.000 |
| BRTHA2009972 | 0.000 | 100.000 |
| BRTHA2010073 | 0.000 | 100.000 |
| BRTHA2010608 | 0.000 | 48.181 |
| BRTHA2010884 | 0.000 | 100.000 |
| BRTHA2010907 | 0.000 | 100.000 |
| BRTHA2011194 | 0.000 | 60.975 |
| BRTHA2011351 | 0.000 | 100.000 |
| BRTHA2011500 | 0.000 | 100.000 |
| BRTHA2011641 | 0.000 | 52.395 |
| BRTHA2012392 | 0.000 | 38.390 |
| BRTHA2012562 | 0.000 | 100.000 |
| BRTHA2012980 | 0.000 | 13.890 |
| BRTHA2013262 | 0.000 | 100.000 |
| BRTHA2013460 | 0.000 | 100.000 |
| BRTHA2013707 | 0.000 | 100.000 |
| BRTHA2014792 | 0.000 | 100.000 |
| BRTHA2014828 | 0.000 | 100.000 |
| BRTHA2015406 | 0.000 | 100.000 |
| BRTHA2015478 | 0.000 | 100.000 |
| BRTHA2015696 | 0.000 | 100.000 |
| BRTHA2015878 | 0.000 | 100.000 |
| BRTHA2016215 | 0.000 | 38.930 |
| BRTHA2016496 | 0.000 | 100.000 |
| BRTHA2016543 | 0.000 | 100.000 |
| BRTHA2017353 | 0.000 | 100.000 |
| BRTHA2017985 | 0.000 | 49.749 |
| BRTHA2018165 | 0.000 | 100.000 |
| BRTHA2018344 | 0.000 | 100.000 |
| BRTHA2018591 | 0.000 | 100.000 |
| BRTHA2018624 | 0.000 | 100.000 |
| BRTHA2018707 | 0.000 | 57.080 |
| BRTHA2019014 | 0.000 | 100.000 |
| BRTHA2019022 | 0.000 | 100.000 |
| BRTHA2019048 | 0.000 | 100.000 |
| BRTHA3000273 | 0.000 | 57.080 |
| BRTHA3000297 | 0.000 | 52.083 |
| BRTHA3000633 | 0.000 | 37.898 |
| BRTHA3001721 | 0.000 | 13.062 |
| BRTHA3002401 | 0.000 | 100.000 |
| BRTHA3002427 | 8.577 | 15.852 |
| BRTHA3002933 | 0.000 | 100.000 |
| BRTHA3003074 | 0.000 | 37.459 |
| BRTHA3003343 | 0.000 | 100.000 |
| BRTHA3003449 | 0.000 | 100.000 |
| BRTHA3003474 | 0.000 | 100.000 |
| BRTHA3003490 | 1.623 | 1.800 |
| BRTHA3004475 | 0.000 | 52.395 |
| BRTHA3005046 | 0.000 | 52.083 |
| BRTHA3006856 | 0.000 | 100.000 |
| BRTHA3007113 | 0.000 | 100.000 |
| BRTHA3007148 | 0.000 | 100.000 |
| BRTHA3007319 | 0.000 | 100.000 |
| BRTHA3007769 | 0.000 | 100.000 |
| BRTHA3008143 | 0.000 | 100.000 |
| BRTHA3008310 | 0.000 | 100.000 |
| BRTHA3008386 | 0.000 | 100.000 |
| BRTHA3008520 | 47.417 | 52.583 |
| BRTHA3008778 | 0.000 | 27.551 |
| BRTHA3009037 | 0.000 | 100.000 |
| BRTHA3009090 | 0.000 | 13.052 |
| BRTHA3009291 | 0.000 | 100.000 |
| BRTHA3010366 | 0.000 | 100.000 |
| BRTHA3013884 | 0.000 | 100.000 |
| BRTHA3015815 | 0.000 | 100.000 |
| BRTHA3015910 | 0.000 | 33.089 |
| BRTHA3016845 | 0.000 | 100.000 |
| BRTHA3016917 | 0.000 | 100.000 |
| BRTHA3017047 | 0.000 | 100.000 |
| BRTHA3017589 | 0.000 | 100.000 |
| BRTHA3017848 | 47.417 | 52.583 |
| BRTHA3018514 | 0.000 | 100.000 |
| BRTHA3018617 | 0.000 | 100.000 |
| BRTHA3018656 | 0.000 | 100.000 |
| BRTHA3019105 | 0.000 | 100.000 |
| COLON2001721 | 11.065 | 0.000 |
| CTONG1000087 | 0.000 | 3.479 |
| CTONG2008233 | 0.000 | 3.046 |
| CTONG2017500 | 2.649 | 2.938 |
| CTONG2019788 | 0.000 | 3.391 |
| CTONG2023021 | 0.000 | 37.320 |
| CTONG2028124 | 0.503 | 1.672 |
| CTONG3000657 | 3.880 | 8.605 |
| CTONG3001123 | 7.847 | 0.000 |
| CTONG3008894 | 0.000 | 2.418 |
| CTONG3009028 | 0.000 | 1.785 |
| CTONG3009239 | 0.000 | 1.601 |
| CTONG3009328 | 11.993 | 0.000 |
| FCBBF2001183 | 16.537 | 9.169 |
| FCBBF3001977 | 7.448 | 0.000 |
| FCBBF3021576 | 0.000 | 19.062 |
| FEBRA2007544 | 14.689 | 2.715 |
| FEBRA2007801 | 4.937 | 5.475 |
| FEBRA2008287 | 0.000 | 6.537 |
| FEBRA2008360 | 0.000 | 11.338 |
| FEBRA2020886 | 12.124 | 0.000 |
| FEBRA2028618 | 5.082 | 0.000 |
| HCASM2007047 | 3.431 | 0.000 |
| HCHON2000028 | 0.000 | 5.278 |
| HCHON2000212 | 0.000 | 19.062 |
| HCHON2000244 | 0.658 | 0.000 |
| HCHON2000626 | 2.351 | 1.304 |
| HCHON2001084 | 0.000 | 2.546 |
| HCHON2001217 | 3.123 | 5.771 |
| HCHON2002676 | 13.647 | 0.000 |
| HCHON2005921 | 0.000 | 9.198 |
| HCHON2006250 | 1.565 | 1.735 |
| HEART1000074 | 0.759 | 0.000 |
| HEART2007031 | 0.000 | 12.601 |
| HHDPC1000118 | 2.096 | 0.000 |
| HLUNG2001996 | 0.000 | 23.216 |
| HLUNG2002465 | 1.209 | 1.341 |
| IMR322000127 | 3.098 | 0.000 |
| IMR322001380 | 0.000 | 2.198 |
| IMR322002035 | 36.176 | 0.000 |
| KIDNE2002252 | 0.000 | 2.417 |
| KIDNE2005543 | 0.000 | 19.748 |
| KIDNE2006580 | 7.013 | 7.777 |
| KIDNE2011314 | 0.000 | 24.133 |
| MESAN2006563 | 0.691 | 1.532 |
| MESAN2012054 | 12.754 | 0.000 |
| MESTC1000042 | 2.245 | 0.000 |
| NOVAR2001783 | 4.027 | 0.000 |
| NT2NE2006909 | 0.285 | 0.000 |
| NT2RI2008724 | 1.836 | 4.073 |
| NT2RI2012659 | 13.562 | 0.000 |
| NT2RI2014733 | 24.938 | 0.000 |
| NT2RI3002842 | 0.000 | 20.781 |
| NT2RI3002892 | 8.799 | 13.661 |
| NT2RI3005403 | 0.000 | 37.898 |
| NT2RI3006284 | 4.138 | 0.000 |
| NT2RI3006673 | 19.959 | 0.000 |
| NT2RI3007543 | 1.697 | 0.000 |
| NT2RI3008055 | 44.179 | 0.000 |
| NT2RP7004123 | 0.000 | 2.128 |
| NT2RP7005529 | 14.766 | 0.000 |
| NT2RP7009147 | 14.337 | 3.533 |
| NT2RP7014005 | 5.818 | 0.000 |
| NT2RP7017474 | 18.823 | 0.000 |
| NTONG2005969 | 0.000 | 3.562 |
| NTONG2008088 | 0.000 | 11.332 |
| OCBBF2001794 | 4.728 | 0.000 |
| OCBBF2006005 | 9.535 | 21.147 |
| OCBBF2006764 | 15.345 | 0.000 |
| OCBBF2007028 | 9.665 | 18.374 |
| OCBBF2010140 | 32.508 | 0.000 |
| OCBBF2020639 | 0.000 | 25.048 |
| OCBBF2021286 | 18.456 | 10.233 |
| OCBBF2024719 | 0.000 | 32.892 |
| OCBBF2024850 | 4.445 | 7.393 |
| OCBBF2028935 | 5.789 | 2.568 |
| OCBBF2036743 | 11.053 | 12.257 |
| OCBBF2038317 | 19.713 | 10.930 |
| OCBBF3000483 | 11.973 | 0.000 |
| OCBBF3008230 | 29.840 | 33.091 |
| PEBLM2004666 | 3.715 | 0.000 |
| PLACE6001185 | 21.358 | 0.000 |
| PUAEN2002489 | 0.000 | 21.707 |
| PUAEN2005930 | 18.362 | 10.181 |
| PUAEN2006701 | 2.249 | 0.000 |
| PUAEN2007044 | 8.600 | 1.590 |
| PUAEN2009655 | 18.275 | 0.000 |
| RECTM2001347 | 0.000 | 4.600 |
| SKMUS2000757 | 0.000 | 5.480 |
| SPLEN2006122 | 0.000 | 0.934 |
| SPLEN2010912 | 0.000 | 13.734 |
| SPLEN2025491 | 0.000 | 38.930 |
| SPLEN2028914 | 2.792 | 0.000 |
| SPLEN2031424 | 15.229 | 8.444 |
| SPLEN2031547 | 1.574 | 1.746 |
| SPLEN2032154 | 0.000 | 38.930 |
| SPLEN2034781 | 27.984 | 0.000 |
| SPLEN2036821 | 0.000 | 38.930 |
| SPLEN2036932 | 2.932 | 0.000 |
| SYNOV1000374 | 0.000 | 22.005 |
| SYNOV2014400 | 12.977 | 0.000 |
| SYNOV4002346 | 5.997 | 0.000 |
| SYNOV4002883 | 23.940 | 0.000 |
| SYNOV4007430 | 31.677 | 0.000 |
| SYNOV4007671 | 0.000 | 0.813 |
| SYNOV4008440 | 1.475 | 3.272 |
| TESOP2002451 | 2.375 | 0.000 |
| TESTI2049246 | 0.000 | 29.159 |
| TESTI4000014 | 1.964 | 1.188 |
| TESTI4000209 | 2.649 | 2.938 |
| TESTI4001100 | 4.098 | 4.544 |
| TESTI4002290 | 0.000 | 62.915 |
| TESTI4006137 | 25.755 | 28.561 |
| TESTI4008797 | 12.429 | 27.567 |
| TESTI4009286 | 1.450 | 0.804 |
| TESTI4010851 | 3.280 | 3.031 |
| TESTI4012702 | 0.000 | 2.417 |
| TESTI4013817 | 27.163 | 0.000 |
| TESTI4014159 | 0.000 | 25.996 |
| TESTI4014694 | 2.229 | 0.000 |
| TESTI4021478 | 22.098 | 49.012 |
| TESTI4022936 | 26.445 | 0.000 |
| TESTI4024420 | 37.398 | 0.000 |
| TESTI4027821 | 60.471 | 0.000 |
| TESTI4037156 | 0.000 | 0.307 |
| THYMU2001090 | 21.252 | 47.135 |
| THYMU2025707 | 0.000 | 20.521 |
| THYMU2032825 | 0.000 | 20.620 |
| THYMU2033308 | 13.964 | 0.000 |
| THYMU2033787 | 0.000 | 57.080 |
| THYMU2035735 | 1.319 | 0.000 |
| THYMU2039315 | 54.530 | 0.000 |
| THYMU2040975 | 0.000 | 1.948 |
| THYMU3001234 | 11.085 | 12.293 |
| THYMU3001379 | 0.000 | 27.929 |
| THYMU3004835 | 0.000 | 21.784 |
| THYMU3008171 | 20.170 | 0.000 |
| TKIDN2009641 | 5.782 | 12.823 |
| TKIDN2009889 | 35.077 | 0.000 |
| TKIDN2015788 | 5.261 | 2.917 |
| TLIVE2001327 | 0.000 | 5.201 |
| TRACH1000205 | 19.677 | 0.000 |
| TRACH2001549 | 8.457 | 4.689 |
| TRACH2005811 | 2.109 | 3.508 |
| TRACH2006049 | 47.167 | 0.000 |
| TRACH2007834 | 0.504 | 0.559 |
| TRACH2008300 | 10.1 [symbol] 6 | 11.296 |
| TRACH2023299 | 0.000 | 14.157 |
| TRACH2025535 | 5.806 | 1.288 |
| TRACH3001427 | 5.571 | 1.030 |
| TRACH3002192 | 4.989 | 2.766 |
| TRACH3004068 | 0.000 | 1.581 |
| TRACH3004721 | 8.721 | 3.224 |
| TRACH3005294 | 7.428 | 0.000 |
| TRACH3007479 | 1.075 | 0.000 |
| TRACH3008093 | 2.449 | 2.716 |
| TRACH3009455 | 47.167 | 0.000 |
| TSTOM1000135 | 0.000 | 9.361 |
| TUTER2000904 | 0.000 | 3.096 |
| UTERU2002410 | 0.000 | 1.302 |
| UTERU2006115 | 7.837 | 17.383 |
| UTERU2019706 | 45.606 | 0.000 |
| UTERU2019940 | 0.000 | 48.181 |
| UTERU2023039 | 45.606 | 0.000 |
| UTERU2023175 | 0.000 | 1.651 |
| UTERU2026203 | 45.606 | 0.000 |
| UTERU2030280 | 0.000 | 48.181 |
| UTERU3000899 | 0.000 | 31.292 |
| UTERU3001571 | 0.000 | 48.181 |
| UTERU3001572 | 0.000 | 6.073 |
| UTERU3004709 | 0.000 | 48.181 |
| UTERU3005230 | 24.419 | 0.000 |
| UTERU3005907 | 0.000 | 14.514 |
| UTERU3007640 | 45.606 | 0.000 |
| UTERU3009871 | 36.230 | 0.000 |
| ADRGL2000042 | 2.540 | 0.000 |
| BLADE2006830 | 1.681 | 0.000 |
| BRACE2003609 | 9.090 | 0.000 |
| BRAMY3004800 | 38.061 | 0.000 |
| BRAWH1000369 | 100.000 | 0.000 |
| BRAWH2006207 | 12.943 | 0.000 |
| BRAWH2006395 | 12.446 | 0.000 |
| BRAWH2008993 | 49.811 | 0.000 |
| BRAWH2009393 | 100.000 | 0.000 |
| BRAWH2010552 | 58.488 | 0.000 |
| BRAWH3007441 | 100.000 | 0.000 |
| BRAWH3009017 | 100.000 | 0.000 |
| BRHIP2005271 | 7.083 | 7.855 |
| BRHIP3000017 | 8.819 | 0.000 |
| BRTHA2002133 | 0.000 | 100.000 |
| BRTHA2002702 | 0.000 | 100.000 |
| BRTHA2007060 | 0.000 | 100.000 |
| BRTHA2010033 | 0.000 | 100.000 |
| BRTHA2011321 | 0.000 | 100.000 |
| BRTHA2013426 | 0.000 | 100.000 |
| BRTHA2013610 | 0.000 | 100.000 |
| BRTHA2016318 | 0.000 | 100.000 |
| BRTHA2017364 | 0.000 | 100.000 |
| BRTHA2017972 | 0.000 | 37.427 |
| BRTHA2018011 | 0.000 | 100.000 |
| BRTHA2018443 | 22.098 | 49.012 |
| BRTHA3000296 | 0.000 | 100.000 |
| BRTHA3003000 | 17.150 | 19.019 |
| BRTHA3008826 | 0.000 | 100.000 |
| CTONG2008721 | 0.000 | 37.320 |
| CTONG2020374 | 31.081 | 0.000 |
| CTONG2020378 | 16.140 | 0.000 |
| CTONG2024031 | 2.584 | 0.000 |
| FCBBF1000509 | 3.732 | 6.208 |
| FEBRA2001990 | 18.144 | 8.048 |
| FEBRA2006519 | 11.891 | 0.000 |
| FEBRA2028516 | 8.007 | 1.268 |
| HCHON2000743 | 6.105 | 6.771 |
| HSYRA2005628 | 0.000 | 7.437 |
| IMR322001879 | 9.638 | 0.000 |
| NT2RI2009583 | 0.224 | 1.489 |
| OCBBF2008144 | 5.768 | 3.198 |
| PERIC2007068 | 3.086 | 1.711 |
| PUAEN2006335 | 12.682 | 0.000 |
| SPLEN2016932 | 0.000 | 38.930 |
| SPLEN2039379 | 5.792 | 3.212 |
| SYNOV2006620 | 0.000 | 33.957 |
| TESTI4001984 | 60.471 | 0.000 |
| TESTI4008058 | 8.814 | 0.000 |
| TESTI4025268 | 60.471 | 0.000 |
| TESTI4032090 | 60.471 | 0.000 |
| THYMU3000360 | 39.314 | 0.000 |
| TLIVE2002046 | 5.445 | 12.076 |
| TRACH3000134 | 36.165 | 0.000 |
| UTERU2021820 | 24.929 | 27.645 |
| UTERU2028734 | 21.953 | 0.000 |

The result of comparative analysis of cDNA libraries derived from the amygdale (BRAMY), and from whole tissues of a normal brain (BRAWH) showed following genes whose expression levels differed between the two.

TABLE 9

| Clone ID | BRAWH | BRAMY |
|---|---|---|
| ASTRO1000009 | 2.611 | 0.000 |
| BLADE2008398 | 12.401 | 0.000 |
| BRACE1000186 | 4.324 | 0.000 |
| BRACE1000258 | 31.956 | 0.000 |
| BRACE1000533 | 11.795 | 4.754 |
| BRACE2005457 | 58.488 | 0.000 |
| BRACE2006319 | 0.000 | 3.532 |
| BRACE2010489 | 63.510 | 10.665 |
| BRACE2014657 | 15.451 | 7.784 |
| BRACE2015058 | 0.000 | 8.836 |
| BRACE2027258 | 0.000 | 34.044 |
| BRACE2030341 | 0.000 | 6.969 |
| BRACE2031389 | 0.000 | 30.747 |
| BRACE20353$$1 | 10.177 | 10.254 |
| BRACE2044286 | 18.667 | 0.000 |
| BRACE2045954 | 27.309 | 0.000 |
| BRACE3000787 | 32.844 | 0.000 |
| BRACE3000973 | 0.000 | 13.581 |
| BRACE3003192 | 58.488 | 0.000 |
| BRACE3005499 | 31.276 | 0.000 |
| BRACE3007480 | 19.471 | 6.540 |
| BRACE3008036 | 0.000 | 18.594 |
| BRACE3009237 | 18.139 | 0.000 |
| BRACE3009724 | 58.488 | 0.000 |
| BRACE3009747 | 2.237 | 0.000 |
| BRACE3010428 | 6.868 | 3.460 |
| BRACE3011271 | 11.036 | 11.120 |
| BRACE3011421 | 28.251 | 0.000 |
| BRACE3012364 | 8.506 | 0.000 |
| BRACE3013780 | 0.000 | 4.833 |
| BRACE3022769 | 4.285 | 4.318 |
| BRACE3026735 | 24.173 | 24.356 |
| BRACE3027478 | 0.000 | 16.126 |
| BRACE3031838 | 58.488 | 0.000 |
| BRALZ2011796 | 5.511 | 0.000 |
| BRAMY2001473 | 0.000 | 100.000 |
| BRAMY2003008 | 26.445 | 26.646 |
| BRAMY2004771 | 0.000 | 100.000 |
| BRAMY2005052 | 11.612 | 46.801 |
| BRAMY2017528 | 0.000 | 100.000 |
| BRAMY2019300 | 49.811 | 50.189 |
| BRAMY2019963 | 20.428 | 6.861 |
| BRAMY2019985 | 0.000 | 100.000 |
| BRAMY2020058 | 0.000 | 28.146 |
| BRAMY2020270 | 0.000 | 100.000 |
| BRAMY2021498 | 0.000 | 100.000 |
| BRAMY2028856 | 0.000 | 100.000 |
| BRAMY2028914 | 0.000 | 17.849 |
| BRAMY2029602 | 0.000 | 100.000 |
| BRAMY2030098 | 0.000 | 42.496 |
| BRAMY2030109 | 0.000 | 100.000 |
| BRAMY2030702 | 0.000 | 100.000 |
| BRAMY2030703 | 0.000 | 100.000 |
| BRAMY2030799 | 0.000 | 100.000 |
| BRAMY2031317 | 0.000 | 15.805 |
| BRAMY2031377 | 0.000 | 100.000 |
| BRAMY2031442 | 0.000 | 100.000 |
| BRAMY2032014 | 0.000 | 100.000 |
| BRAMY2032242 | 0.000 | 100.000 |
| BRAMY2032317 | 0.000 | 60.652 |
| BRAMY2033003 | 0.000 | 100.000 |
| BRAMY2033116 | 0.000 | 100.000 |
| BRAMY2033267 | 0.000 | 100.000 |
| BRAMY2033594 | 0.000 | 100.000 |
| BRAMY2034185 | 0.000 | 100.000 |
| BRAMY2034920 | 0.000 | 100.000 |
| BRAMY2034993 | 0.000 | 100.000 |
| BRAMY2036387 | 0.000 | 13.490 |
| BRAMY2036396 | 0.000 | 100.000 |
| BRAMY2036567 | 7.474 | 22.594 |
| BRAMY2036699 | 0.000 | 100.000 |
| BRAMY2036913 | 0.000 | 100.000 |
| BRAMY2037823 | 29.664 | 29.890 |
| BRAMY2038100 | 0.000 | 100.000 |
| BRAMY2038484 | 0.000 | 100.000 |
| BRAMY2038846 | 0.000 | 47.355 |
| BRAMY2038904 | 0.000 | 60.652 |
| BRAMY2039872 | 0.000 | 19.976 |
| BRAMY2040478 | 0.000 | 100.000 |
| BRAMY2040592 | 3.482 | 7.017 |
| BRAMY2041261 | 0.000 | 100.000 |
| BRAMY2041378 | 0.000 | 100.000 |
| BRAMY2041542 | 0.000 | 100.000 |
| BRAMY2042612 | 0.000 | 100.000 |
| BRAMY2042641 | 0.000 | 100.000 |
| BRAMY2042760 | 0.000 | 100.000 |
| BRAMY2042918 | 0.000 | 100.000 |
| BRAMY2044078 | 0.000 | 8.836 |
| BRAMY2044246 | 0.000 | 100.000 |
| BRAMY2045036 | 0.000 | 100.000 |
| BRAMY2046478 | 0.000 | 100.000 |
| BRAMY2046742 | 0.000 | 100.000 |
| BRAMY2046989 | 0.000 | 100.000 |
| BRAMY2047169 | 0.000 | 100.000 |
| BRAMY2047420 | 0.000 | 2.041 |
| BRAMY2047676 | 0.000 | 100.000 |
| BRAMY2047746 | 0.000 | 100.000 |
| BRAMY2047751 | 0.000 | 100.000 |
| BRAMY2047765 | 0.000 | 100.000 |
| BRAMY2047884 | 0.000 | 100.000 |
| BRAMY3000206 | 0.000 | 100.000 |
| BRAMY3000213 | 0.000 | 100.000 |
| BRAMY3001401 | 0.000 | 100.000 |
| BRAMY3001794 | 0.000 | 100.000 |
| BRAMY3002312 | 0.000 | 100.000 |
| BRAMY3002620 | 0.000 | 58.671 |
| BRAMY3002803 | 14.428 | 14.538 |
| BRAMY3002805 | 0.000 | 100.000 |
| BRAMY3004224 | 33.027 | 33.278 |
| BRAMY3004672 | 0.000 | 100.000 |
| BRAMY3004900 | 0.000 | 100.000 |
| BRAMY3004919 | 0.000 | 100.000 |
| BRAMY3005091 | 19.193 | 19.339 |
| BRAMY3005932 | 0.000 | 58.671 |
| BRAMY3006297 | 0.000 | 100.000 |
| BRAMY3007206 | 0.000 | 100.000 |
| BRAMY3007609 | 0.000 | 100.000 |
| BRAMY3008466 | 0.000 | 100.000 |
| BRAMY3008505 | 0.000 | 100.000 |
| BRAMY3008650 | 0.000 | 100.000 |
| BRAMY3009811 | 0.000 | 33.057 |
| BRAMY3010411 | 0.000 | 100.000 |
| BRAMY4000095 | 0.000 | 14.721 |
| BRAMY4000229 | 0.000 | 35.650 |
| BRAMY4000277 | 0.000 | 100.000 |
| BRAWH1000127 | 15.983 | 9.663 |
| BRAWH2001395 | 14.290 | 2.541 |
| BRAWH2001671 | 7.605 | 0.000 |
| BRAWH2001940 | 37.398 | 0.000 |
| BRAWH2001973 | 37.398 | 0.000 |
| BRAWH2002560 | 6.454 | 6.503 |
| BRAWH2002761 | 100.000 | 0.000 |
| BRAWH2005315 | 100.000 | 0.000 |
| BRAWH2007658 | 58.101 | 0.000 |
| BRAWH2010000 | 18.745 | 0.000 |
| BRAWH2010084 | 100.000 | 0.000 |
| BRAWH2010536 | 14.718 | 0.000 |
| BRAWH2012162 | 36.060 | 0.000 |
| BRAWH2012326 | 100.000 | 0.000 |
| BRAWH2013294 | 39.442 | 0.000 |
| BRAWH2013871 | 37.485 | 0.000 |
| BRAWH2014414 | 17.865 | 54.002 |
| BRAWH2014645 | 4.228 | 2.130 |
| BRAWH2014662 | 15.521 | 0.000 |
| BRAWH2014876 | 10.473 | 0.000 |
| BRAWH2014954 | 58.488 | 0.000 |
| BRAWH2016221 | 47.417 | 0.000 |
| BRAWH2016439 | 100.000 | 0.000 |
| BRAWH2016702 | 73.807 | 0.000 |
| BRAWH2016724 | 35.119 | 0.000 |
| BRAWH3000078 | 100.000 | 0.000 |
| BRAWH3000100 | 100.000 | 0.000 |
| BRAWH3000314 | 71.553 | 0.000 |
| BRAWH3000491 | 100.000 | 0.000 |
| BRAWH3001326 | 45.606 | 0.000 |
| BRAWH3001475 | 100.000 | 0.000 |
| BRAWH3001891 | 34.539 | 17.401 |
| BRAWH3002574 | 13.222 | 0.000 |
| BRAWH3002600 | 36.800 | 37.080 |
| BRAWH3002819 | 100.000 | 0.000 |
| BRAWH3002821 | 21.953 | 0.000 |
| BRAWH3003522 | 100.000 | 0.000 |
| BRAWH3003555 | 15.229 | 0.000 |
| BRAWH3003727 | 10.055 | 0.000 |
| BRAWH3003801 | 100.000 | 0.000 |
| BRAWH3003992 | 29.008 | 0.000 |
| BRAWH3004453 | 100.000 | 0.000 |
| BRAWH3004666 | 49.499 | 0.000 |
| BRAWH3005132 | 49.811 | 50.189 |
| BRAWH3005422 | 100.000 | 0.000 |
| BRAWH3005912 | 100.000 | 0.000 |
| BRAWH3005981 | 29.324 | 0.000 |
| BRAWH3006548 | 71.018 | 0.000 |
| BRAWH3006792 | 49.499 | 0.000 |
| BRAWH3007221 | 100.000 | 0.000 |
| BRAWH3007506 | 100.000 | 0.000 |
| BRAWH3007592 | 8.966 | 1.807 |
| BRAWH3007726 | 54.530 | 0.000 |
| BRAWH3007783 | 100.000 | 0.000 |
| BRAWH3008341 | 100.000 | 0.000 |
| BRAWH3008697 | 100.000 | 0.000 |
| BRAWH3008931 | 3.463 | 6.980 |
| BRAWH3009297 | 58.488 | 0.000 |
| BRCAN2014881 | 0.000 | 30.516 |
| BRCAN2017717 | 0.000 | 10.719 |
| BRCOC2000670 | 0.000 | 22.144 |
| BRCOC2003213 | 10.381 | 0.000 |
| BRCOC2014033 | 15.633 | 0.000 |
| BRCOC2020142 | 22.014 | 0.000 |
| BRHIP2000920 | 36.630 | 0.000 |
| BRHIP2005719 | 49.499 | 0.000 |
| BRHIP2007741 | 6.943 | 8.745 |
| BRHIP2014228 | 29.439 | 0.000 |
| BRHIP2024146 | 3.042 | 2.759 |
| BRHIP2026061 | 0.000 | 30.812 |
| BRHIP3000339 | 14.290 | 2.541 |
| BRHIP3001283 | 0.000 | 49.688 |
| BRHIP3006683 | 24.100 | 0.000 |
| BRHIP3007586 | 17.255 | 3.477 |
| BRHIP3008405 | 35.187 | 0.000 |
| BRHIP3018797 | 30.810 | 9.448 |
| BRSSN2000684 | 23.433 | 0.000 |
| BRSSN2004496 | 0.000 | 21.304 |
| BRSSN2011738 | 31.553 | 0.000 |
| BRSSN2014299 | 3.695 | 0.000 |
| BRSTN2008052 | 32.844 | 0.000 |
| BRSTN2010750 | 0.000 | 30.747 |
| BRSTN2015015 | 14.017 | 0.000 |
| BRSTN2016470 | 0.438 | 0.441 |
| BRTHA1000311 | 11.803 | 14.866 |
| BRTHA2008335 | 16.281 | 0.000 |
| BRTHA2011641 | 0.000 | 47.605 |
| BRTHA3001721 | 0.000 | 23.737 |
| BRTHA3002427 | 8.577 | 5.761 |
| BRTHA3003490 | 1.623 | 0.000 |
| BRTHA3004475 | 0.000 | 47.605 |
| BRTHA3008520 | 47.417 | 0.000 |
| BRTHA3009090 | 0.000 | 11.859 |
| BRTHA3017848 | 47.417 | 0.000 |
| COLON2001721 | 11.065 | 11.149 |
| CTONG1000087 | 0.000 | 3.161 |
| CTONG2008233 | 0.000 | 0.692 |
| CTONG2017500 | 2.649 | 2.669 |
| CTONG2028124 | 0.503 | 0.506 |
| CTONG3000657 | 3.880 | 0.000 |
| CTONG3001123 | 7.847 | 0.000 |
| CTONG3008894 | 0.000 | 4.395 |
| CTONG3009239 | 0.000 | 4.364 |
| CTONG3009328 | 11.993 | 0.000 |
| FCBBF20011$$3 | 16.537 | 9.998 |
| FCBBF3001977 | 7.448 | 15.010 |
| FEBRA2007544 | 14.689 | 0.000 |
| FEBRA2007801 | 4.937 | 0.000 |
| FEBRA2008287 | 0.000 | 2.970 |
| FEBRA2010719 | 0.000 | 17.484 |
| FEBRA2020886 | 12.124 | 0.000 |
| FEBRA2025427 | 0.000 | 4.995 |
| FEBRA2028618 | 5.082 | 5.121 |
| HCASM2007047 | 3.431 | 0.000 |
| HCHON2000244 | 0.658 | 0.663 |
| HCHON2000626 | 2.351 | 0.000 |
| HCHON2001217 | 3.123 | 0.000 |
| HCHON2002676 | 13.647 | 0.000 |
| HCHON2006250 | 1.565 | 0.000 |
| HCHON2008112 | 0.000 | 13.736 |
| HEART1000074 | 0.759 | 0.000 |
| HHDPC1000118 | 2.096 | 0.000 |
| HLUNG2002465 | 1.209 | 4.875 |
| HSYRA2009075 | 0.000 | 1.212 |
| IMR322000127 | 3.098 | 10.926 |
| IMR322001380 | 0.000 | 11.984 |
| IMR322002035 | 36.176 | 0.000 |
| KIDNE2000665 | 0.000 | 1.951 |
| KIDNE2006580 | 7.013 | 7.066 |
| MESAN2006563 | 0.691 | 0.000 |
| MESAN2012054 | 12.754 | 0.000 |
| MESAN2015515 | 0.000 | 2.677 |
| MESTC1000042 | 2.245 | 0.000 |
| NOVAR2001783 | 4.027 | 0.000 |
| NT2NE2005890 | 0.000 | 35.735 |
| NT2NE2006909 | 0.285 | 0.287 |
| NT2RI2008724 | 1.836 | 1.850 |
| NT2RI2012659 | 13.562 | 13.665 |
| NT2RI2014733 | 24.938 | 0.000 |
| NT2RI3001515 | 0.000 | 21.825 |
| NT2RI3002$$92 | 8.799 | 1.773 |
| NT2RI3005724 | 0.000 | 5.972 |
| NT2RI3006284 | 4.138 | 0.000 |
| NT2RI3006673 | 19.959 | 10.055 |
| NT2RI3007543 | 1.697 | 0.855 |
| NT2RI3008055 | 44.179 | 0.000 |
| NT2RP7005529 | 14.766 | 7.439 |
| NT2RP7009147 | 14.337 | 0.000 |
| NT2RP7014005 | 5.818 | 0.000 |
| NT2RP7017474 | 18.823 | 0.000 |
| NTONG2005969 | 0.000 | 6.473 |
| OCBBF1000254 | 0.000 | 44.784 |
| OCBBF2001794 | 4.728 | 4.764 |
| OCBBF2006005 | 9.535 | 0.000 |
| OCBBF2006764 | 15.345 | 15.461 |
| OCBBF2007028 | 9.665 | 6.956 |
| OCBBF2007114 | 0.000 | 21.540 |
| OCBBF2010140 | 32.508 | 0.000 |
| OCBBF2021286 | 18.456 | 0.000 |
| OCBBF2023162 | 0.000 | 31.080 |
| OCBBF2024850 | 4.445 | 2.239 |
| OCBBF2028935 | 5.789 | 1.167 |
| OCBBF2035214 | 0.000 | 44.784 |
| OCBBF2036743 | 11.053 | 11.137 |
| OCBBF2038317 | 19.713 | 0.000 |
| OCBBF3000483 | 11.973 | 4.021 |
| OCBBF3008230 | 29.840 | 0.000 |
| PEBLM2004666 | 3.715 | 3.744 |
| PERIC2000889 | 0.000 | 8.963 |
| PERIC2003720 | 0.000 | 13.012 |
| PLACE6001185 | 21.358 | 0.000 |
| PUAEN2005930 | 18.362 | 0.000 |
| PUAEN2006701 | 2.249 | 0.000 |
| PUAEN2007044 | 8.600 | 1.444 |
| PUAEN2009174 | 0.000 | 2.856 |
| PUAEN2009655 | 18.275 | 0.000 |
| SKNMC2002402 | 0.000 | 7.428 |
| SKNSH2000482 | 0.000 | 9.821 |
| SPLEN2001599 | 0.000 | 7.190 |
| SPLEN2002467 | 0.000 | 6.565 |
| SPLEN2028914 | 2.792 | 0.000 |
| SPLEN2029912 | 0.000 | 1.646 |
| SPLEN2031424 | 15.229 | 0.000 |
| SPLEN2031547 | 1.574 | 4.759 |
| SPLEN2034781 | 27.984 | 0.000 |
| SPLEN2036932 | 2.932 | 0.000 |
| SPLEN2038345 | 0.000 | 29.627 |
| SYNOV2014400 | 12.977 | 0.000 |
| SYNOV4002346 | 5.997 | 0.000 |
| SYNOV4002883 | 23.940 | 0.000 |
| SYNOV4007430 | 31.677 | 0.000 |
| SYNOV4007671 | 0.000 | 0.739 |
| SYNOV4008440 | 1.475 | 0.000 |
| TESOP2002451 | 2.375 | 0.000 |
| TESTI2009474 | 0.000 | 2.036 |
| TESTI4000014 | 1.964 | 1.799 |
| TESTI4000209 | 2.649 | 2.669 |
| TESTI4001100 | 4.098 | 4.129 |
| TESTI4006137 | 25.755 | 0.000 |
| TESTI4008797 | 12.429 | 0.000 |
| TESTI4009286 | 1.450 | 0.000 |
| TESTI4010851 | 3.280 | 2.203 |
| TESTI4013817 | 27.163 | 27.369 |
| TESTI4014159 | 0.000 | 11.810 |
| TESTI4014694 | 2.229 | 0.000 |
| TESTI4021478 | 22.098 | 0.000 |
| TESTI4022936 | 26.445 | 26.646 |
| TESTI4024420 | 37.398 | 0.000 |
| TESTI4027821 | 60.471 | 0.000 |
| TESTI4029836 | 0.000 | 60.652 |
| TESTI4037156 | 0.000 | 1.394 |
| TESTI4037188 | 0.000 | 60.652 |
| THYMU2001090 | 21.252 | 0.000 |
| THYMU2014353 | 0.000 | 37.663 |
| THYMU2033308 | 13.964 | 0.000 |
| THYMU2035735 | 1.319 | 0.000 |
| THYMU2037226 | 0.000 | 17.265 |
| THYMU2039315 | 54.530 | 0.000 |
| THYMU3001234 | 11.085 | 0.000 |
| THYMU3001379 | 0.000 | 25.376 |
| THYMU3004835 | 0.000 | 19.793 |
| THYMU3008171 | 20.170 | 0.000 |
| TKIDN2009641 | 5.782 | 5.826 |
| TKIDN2009889 | 35.077 | 0.000 |
| TKIDN2015788 | 5.261 | 5.301 |
| TLIVE2004320 | 0.000 | 12.783 |
| TRACH1000205 | 19.677 | 3.965 |
| TRACH2001549 | 8.457 | 4.260 |
| TRACH2001684 | 0.000 | 17.948 |
| TRACH2005811 | 2.109 | 0.000 |
| TRACH2006049 | 47.167 | 0.000 |
| TRACH2007834 | 0.504 | 1.016 |
| TRACH2008300 | 10.186 | 11.974 |
| TRACH2025344 | 0.000 | 19.846 |
| TRACH2025535 | 5.806 | 0.000 |
| TRACH2025911 | 0.000 | 47.355 |
| TRACH3001427 | 5.571 | 3.742 |
| TRACH3002192 | 4.989 | 1.885 |
| TRACH3004068 | 0.000 | 1.436 |
| TRACH3004721 | 8.721 | 5.858 |
| TRACH3005294 | 7.428 | 0.000 |
| TRACH3007479 | 1.075 | 1.084 |
| TRACH3008093 | 2.449 | 0.000 |
| TRACH3009455 | 47.167 | 0.000 |
| TUTER2000904 | 0.000 | 2.813 |
| UTERU2002410 | 0.000 | 0.592 |
| UTERU2004929 | 0.000 | 11.697 |
| UTERU2006115 | 7.837 | 0.000 |
| UTERU2007520 | 0.000 | 6.758 |
| UTERU2019706 | 45.606 | 0.000 |
| UTERU2023039 | 45.606 | 0.000 |
| UTERU2026203 | 45.606 | 0.000 |
| UTERU3001572 | 0.000 | 1.839 |
| UTERU3001766 | 0.000 | 45.794 |
| UTERU3005230 | 24.419 | 0.000 |
| UTERU3007640 | 45.606 | 0.000 |
| UTERU3009517 | 0.000 | 45.794 |
| UTERU3009871 | 36.230 | 0.000 |
| ADRGL2000042 | 2.540 | 5.118 |
| BLADE2006830 | 1.681 | 0.000 |
| BRACE2003609 | 9.090 | 18.317 |
| BRACE2039823 | 0.000 | 58.671 |
| BRAMY2019111 | 0.000 | 30.516 |
| BRAMY2035070 | 0.000 | 54.718 |
| BRAMY2035449 | 0.000 | 100.000 |
| BRAMY2035718 | 0.000 | 100.000 |
| BRAMY2038516 | 0.000 | 12.770 |
| BRAMY2039341 | 0.000 | 100.000 |
| BRAMY2040159 | 0.000 | 100.000 |
| BRAMY2041434 | 0.000 | 100.000 |
| BRAMY2045471 | 0.000 | 100.000 |
| BRAMY3004800 | 38.061 | 15.340 |
| BRAWH1000369 | 100.000 | 0.000 |
| BRAWH2006207 | 12.943 | 0.000 |
| BRAWH2006395 | 12.446 | 0.000 |
| BRAWH2008993 | 49.811 | 50.189 |
| BRAWH2009393 | 100.000 | 0.000 |
| BRAWH2010552 | 58.488 | 0.000 |
| BRAWH3007441 | 100.000 | 0.000 |
| BRAWH3009017 | 100.000 | 0.000 |
| BRHIP2005271 | 7.083 | 7.137 |
| BRHIP3000017 | 8.819 | 0.000 |
| BRTHA2018443 | 22.098 | 0.000 |
| BRTHA3003000 | 17.150 | 0.000 |
| CTONG2020374 | 31.081 | 0.000 |
| CTONG2020378 | 16.140 | 0.000 |
| CTONG2024031 | 2.584 | 0.000 |
| FCBBF1000509 | 3.732 | 13.160 |
| FEBRA2001990 | 18.144 | 7.313 |
| FEBRA2006519 | 11.891 | 11.982 |
| FEBRA2028516 | 8.007 | 2.305 |
| HCHON2000508 | 0.000 | 0.376 |
| HCHON2000743 | 6.105 | 0.000 |
| IMR322001879 | 9.638 | 0.000 |
| NT2RI2009583 | 0.224 | 0.902 |
| OCBBF2008144 | 5.768 | 2.906 |
| PERIC2007068 | 3.086 | 0.000 |
| PUAEN2006335 | 12.682 | 0.000 |
| SPLEN2039379 | 5.792 | 11.672 |
| TESTI2015626 | 0.000 | 0.331 |
| TESTI2026647 | 0.000 | 23.811 |
| TESTI4001984 | 60.471 | 0.000 |
| TESTI4008058 | 8.814 | 0.000 |
| TESTI4013894 | 0.000 | 14.112 |
| TESTI4025268 | 60.471 | 0.000 |
| TESTI4032090 | 60.471 | 0.000 |
| THYMU3000360 | 39.314 | 0.000 |
| TKIDN2018926 | 0.000 | 21.396 |
| TLIVE2002046 | 5.445 | 10.972 |
| TRACH3000134 | 36.165 | 0.000 |
| UTERU2008040 | 0.000 | 11.858 |
| UTERU2021820 | 24.929 | 0.000 |
| UTERU2028734 | 21.953 | 0.000 |

The result of comparative analysis of cDNA libraries derived from breast tumor (TBAES), and normal breast (BEAST) showed the following genes whose expression levels differed between the two.

TABLE 10

| Clone ID | BEAST | TBAES |
|---|---|---|
| ASTRO2002842 | 0.000 | 67.001 |
| BRACE3016884 | 0.000 | 90.835 |
| BRSSN2011262 | 61.476 | 0.000 |
| BRTHA2008335 | 0.000 | 57.267 |
| HCHON2000244 | 0.000 | 4.627 |
| HCHON2006250 | 0.000 | 11.007 |
| HEART1000010 | 0.000 | 24.034 |
| MESAN2012054 | 0.000 | 35.889 |
| NT2RP7000466 | 0.000 | 85.728 |
| NT2RP7009147 | 0.000 | 11.206 |
| OCBBF2021020 | 0.000 | 18.756 |
| PEBLM2002749 | 0.000 | 48.344 |
| PEBLM2004666 | 0.000 | 26.137 |
| SPLEN2001599 | 0.000 | 50.196 |
| SPLEN2031547 | 0.000 | 11.076 |
| STOMA1000189 | 0.000 | 22.002 |
| TBAES2001171 | 0.000 | 100.000 |
| TBAES2001220 | 0.000 | 100.000 |
| TBAES2001229 | 0.000 | 100.000 |
| TBAES2001258 | 0.000 | 66.863 |
| TBAES2001492 | 0.000 | 100.000 |
| TBAES2001751 | 0.000 | 100.000 |
| TBAES2002197 | 0.000 | 54.528 |
| TBAES2003550 | 0.000 | 100.000 |
| TBAES2004055 | 0.000 | 100.000 |
| TBAES2005157 | 0.000 | 100.000 |
| TBAES2005543 | 0.000 | 100.000 |
| TBAES2006568 | 0.000 | 100.000 |
| TBAES2007964 | 0.000 | 100.000 |
| TESTI4000014 | 0.000 | 1.256 |
| TESTI4037156 | 0.000 | 3.893 |
| TRACH3002192 | 0.000 | 4.387 |
| TRACH3004068 | 0.000 | 10.026 |
| TSTOM2000553 | 0.000 | 11.859 |
| UTERU2002410 | 0.000 | 8.261 |
| BRAWH2006395 | 0.000 | 87.554 |
| NT2RI2009583 | 4.881 | 0.000 |
| STOMA2004893 | 0.000 | 50.829 |
| TBAES2000932 | 0.000 | 100.000 |

The result of comparative analysis of cDNA libraries derived cervical tumor (TCERX), and normal cervical duct (CERVX) showed the following genes whose expression levels differed between the two.

TABLE 11

| Clone ID | CERVX | TCERX |
|---|---|---|
| BLADE2007666 | 70.405 | 0.000 |
| BRAMY2047420 | 0.000 | 21.378 |
| BRCAN2007409 | 0.000 | 63.318 |
| BRSTN2016470 | 0.000 | 9.238 |
| CERVX1000042 | 100.000 | 0.000 |
| CERVX2002006 | 100.000 | 0.000 |
| MESAN2006563 | 14.270 | 0.000 |
| PROST2018090 | 44.955 | 0.000 |
| TCERX2000613 | 0.000 | 85.503 |
| TESTI4037156 | 0.000 | 5.840 |
| THYMU2031341 | 71.304 | 0.000 |
| UTERU2004688 | 22.977 | 70.393 |
| CERVX2002013 | 100.000 | 0.000 |
| NT2RI2009583 | 0.000 | 9.443 |

The result of comparative analysis of cDNA libraries derived from colon tumor (TCOLN), and normal colon (COLON) showed the following genes whose expression levels differed between the two.

TABLE 12

| Clone ID | COLON | TCOLN |
|---|---|---|
| BRACE3015027 | 0.000 | 55.019 |
| BRAMY2040592 | 24.447 | 0.000 |
| BRSTN2016470 | 6.147 | 9.281 |
| COLON1000030 | 100.000 | 0.000 |
| COLON2000470 | 50.006 | 0.000 |
| COLON2000568 | 100.000 | 0.000 |
| COLON2001721 | 38.840 | 0.000 |
| COLON2002443 | 31.658 | 0.000 |
| COLON2002520 | 100.000 | 0.000 |
| COLON2003043 | 100.000 | 0.000 |
| COLON2004478 | 100.000 | 0.000 |
| COLON2005126 | 100.000 | 0.000 |
| COLON2005772 | 100.000 | 0.000 |
| COLON2006282 | 100.000 | 0.000 |
| COLON2009499 | 100.000 | 0.000 |
| OCBBF2028935 | 8.128 | 0.000 |
| PLACE7000514 | 33.393 | 0.000 |
| RECTM2000433 | 24.395 | 0.000 |
| SYNOV4007671 | 5.146 | 0.000 |
| TCOLN2002278 | 0.000 | 100.000 |
| TESTI2052693 | 23.507 | 35.495 |
| TESTI4037156 | 1.943 | 0.000 |
| THYMU2031368 | 0.000 | 96.216 |
| TRACH2025535 | 16.304 | 0.000 |
| CTONG1000113 | 79.033 | 0.000 |
| NT2RI2009583 | 0.000 | 4.744 |
| NT2RI2018448 | 44.206 | 0.000 |
| TESTI2015626 | 2.309 | 0.000 |

The result of comparative analysis of cDNA libraries derived from esophageal tumor (TESOP), and normal esophagus (NESOP) showed the following genes whose expression levels differed between the two.

TABLE 13

| Clone ID | NESOP | TESOP |
|---|---|---|
| BRACE2030341 | 0.000 | 47.323 |
| BRAMY2047420 | 21.553 | 0.000 |
| BRHIP2003917 | 0.000 | 80.343 |
| BRTHA2003461 | 0.000 | 15.017 |
| CTONG2013178 | 0.000 | 64.748 |
| D3OST3000169 | 0.000 | 6.696 |
| FEBRA2025427 | 0.000 | 16.960 |
| HCHON2000244 | 0.000 | 4.501 |
| HHDPC1000118 | 0.000 | 14.339 |
| NESOP2000744 | 100.000 | 0.000 |
| NESOP2001433 | 100.000 | 0.000 |
| NESOP2001656 | 85.181 | 0.000 |
| NESOP2001694 | 100.000 | 0.000 |
| NESOP2001752 | 96.772 | 0.000 |
| NESOP2002738 | 100.000 | 0.000 |
| NT2RI3006284 | 0.000 | 14.156 |
| NT2RP7009147 | 33.894 | 0.000 |
| PLACE6019932 | 92.281 | 0.000 |
| SYNOV2005216 | 0.000 | 66.809 |
| TESOP1000127 | 0.000 | 65.101 |
| TESOP2000801 | 0.000 | 100.000 |
| TESOP2001122 | 0.000 | 100.000 |
| TESOP2001166 | 0.000 | 100.000 |
| TESOP2001345 | 0.000 | 100.000 |
| TESOP2001605 | 0.000 | 66.809 |
| TESOP2001818 | 0.000 | 100.000 |
| TESOP2001849 | 0.000 | 100.000 |
| TESOP2001865 | 0.000 | 100.000 |
| TESOP2001953 | 0.000 | 89.137 |
| TESOP2002273 | 0.000 | 27.770 |
| TESOP2002451 | 0.000 | 16.251 |
| TESOP2002489 | 0.000 | 100.000 |
| TESOP2002539 | 0.000 | 100.000 |
| TESOP2002950 | 0.000 | 90.601 |
| TESOP2003273 | 0.000 | 100.000 |
| TESOP2003753 | 0.000 | 100.000 |
| TESOP2004114 | 0.000 | 100.000 |
| TESOP2005285 | 0.000 | 100.000 |
| TESOP2005485 | 0.000 | 100.000 |
| TESOP2005579 | 0.000 | 100.000 |
| TESOP2006041 | 0.000 | 100.000 |
| TESOP2006060 | 0.000 | 100.000 |
| TESOP2006068 | 0.000 | 100.000 |
| TESOP2006670 | 0.000 | 79.728 |
| TESOP2006746 | 0.000 | 100.000 |
| TESOP2007052 | 0.000 | 100.000 |
| TESOP2007262 | 0.000 | 100.000 |
| TESOP2007636 | 0.000 | 100.000 |
| TESOP2007688 | 0.000 | 100.000 |
| TESOP2009121 | 0.000 | 100.000 |
| TESOP2009555 | 0.000 | 100.000 |
| TESTI4009286 | 15.424 | 9.920 |
| TESTI4010851 | 0.000 | 3.740 |
| THYMU2040975 | 0.000 | 12.019 |
| TRACH2005811 | 0.000 | 14.431 |
| UTERU2023175 | 0.000 | 10.186 |
| CTONG2016942 | 0.000 | 78.602 |
| NT2RI2009583 | 0.000 | 1.531 |
| TESOP2000390 | 0.000 | 100.000 |
| TESOP2001796 | 0.000 | 100.000 |
| TESOP2005199 | 0.000 | 100.000 |
| TESOP2006398 | 0.000 | 100.000 |
| TESOP2006865 | 0.000 | 100.000 |
| TESOP2007384 | 0.000 | 13.734 |
| TESTI2015626 | 0.000 | 2.250 |
| TRACH2000862 | 0.000 | 39.606 |

The result of comparative analysis of cDNA libraries derived from kidney tumor (TKIDN), and normal kidney (KIDNE) showed the following genes whose expression levels differed between the two.

TABLE 14

| Clone ID | KIDNE | TKIDN |
|---|---|---|
| ASTRO2018373 | 0.000 | 51.654 |
| BRACE1000186 | 0.000 | 16.007 |
| BRACE2014306 | 0.000 | 18.017 |
| BRACE2015058 | 0.000 | 16.230 |
| BRACE2016981 | 0.000 | 83.911 |
| BRACE2043665 | 83.085 | 0.000 |
| BRACE3008036 | 0.000 | 68.309 |
| BRACE3010428 | 3.991 | 0.000 |
| BRACE3022769 | 0.000 | 5.287 |
| BRAMY2019963 | 0.000 | 25.206 |
| BRAMY2044078 | 0.000 | 16.230 |
| BRAWH1000127 | 0.000 | 11.833 |
| BRAWH2001395 | 0.000 | 9.335 |
| BRAWH2001671 | 0.000 | 14.075 |
| BRAWH2013294 | 0.000 | 36.500 |
| BRAWH2014645 | 0.000 | 3.913 |
| BRHIP2024146 | 0.000 | 7.882 |
| BRHIP3000339 | 0.000 | 9.335 |
| BRSSN2000684 | 0.000 | 17.348 |
| BRSSN2004719 | 0.000 | 19.395 |
| BRSSN2018581 | 0.000 | 49.863 |
| BRSTN2016470 | 0.763 | 0.810 |
| BRTHA1000311 | 0.000 | 10.923 |
| BRTHA3002427 | 0.000 | 10.582 |
| CTONG1000087 | 3.646 | 0.000 |
| CTONG2028124 | 3.504 | 0.000 |
| CTONG3000657 | 0.000 | 14.362 |
| CTONG3008894 | 2.534 | 0.000 |
| FCBBF2001183 | 0.000 | 12.243 |
| FEBRA2008287 | 0.000 | 10.911 |
| HCASM2001301 | 0.000 | 35.796 |
| HCHON2000028 | 0.000 | 5.873 |
| HCHON2000244 | 4.587 | 0.000 |
| HEART1000074 | 2.646 | 0.000 |
| HHDPC1000118 | 7.307 | 0.000 |
| HSYRA2008376 | 0.000 | 5.788 |
| KIDNE1000064 | 100.000 | 0.000 |
| KIDNE2000665 | 13.498 | 0.000 |
| KIDNE2000722 | 100.000 | 0.000 |
| KIDNE2000832 | 32.592 | 0.000 |
| KIDNE2000846 | 100.000 | 0.000 |
| KIDNE2001361 | 80.699 | 0.000 |
| KIDNE2001847 | 12.687 | 0.000 |
| KIDNE2002252 | 7.600 | 0.000 |
| KIDNE2002991 | 100.000 | 0.000 |
| KIDNE2003837 | 100.000 | 0.000 |
| KIDNE2005543 | 62.084 | 0.000 |
| KIDNE2006580 | 48.900 | 0.000 |
| KIDNE2010264 | 100.000 | 0.000 |
| KIDNE2011314 | 75.867 | 0.000 |
| KIDNE2011532 | 100.000 | 0.000 |
| KIDNE2011635 | 48.502 | 51.498 |
| KIDNE2012945 | 48.732 | 0.000 |
| KIDNE2013095 | 100.000 | 0.000 |
| NESOP2001656 | 0.000 | 14.819 |
| NTONG2005969 | 0.000 | 11.891 |
| PEBLM2004666 | 12.953 | 0.000 |
| SKMUS2000757 | 0.000 | 18.292 |
| STOMA1000189 | 16.356 | 0.000 |
| SYNOV4007671 | 2.556 | 5.427 |
| TBAES2001258 | 33.137 | 0.000 |
| TESTI4000014 | 1.245 | 0.000 |
| TESTI4001100 | 0.000 | 15.168 |
| TESTI4012702 | 7.600 | 0.000 |
| TESTI4046819 | 0.000 | 73.082 |
| THYMU2032014 | 57.532 | 0.000 |
| TKIDN2000701 | 0.000 | 84.991 |
| TKIDN2002424 | 0.000 | 100.000 |
| TKIDN2002632 | 0.000 | 100.000 |
| TKIDN2003044 | 0.000 | 100.000 |
| TKIDN2004386 | 0.000 | 100.000 |
| TKIDN2005934 | 0.000 | 100.000 |
| TKIDN2005947 | 0.000 | 100.000 |
| TKIDN2006525 | 0.000 | 100.000 |
| TKIDN2006852 | 0.000 | 100.000 |
| TKIDN2007667 | 0.000 | 100.000 |
| TKIDN2009092 | 0.000 | 100.000 |
| TKIDN2009641 | 0.000 | 21.402 |
| TKIDN2009889 | 0.000 | 64.923 |
| TKIDN2010934 | 0.000 | 74.873 |
| TKIDN2012824 | 0.000 | 100.000 |
| TKIDN2013287 | 0.000 | 64.067 |
| TKIDN2014757 | 0.000 | 100.000 |
| TKIDN2014771 | 0.000 | 100.000 |
| TKIDN2015263 | 0.000 | 100.000 |
| TKIDN2015788 | 0.000 | 9.737 |
| TKIDN2016309 | 0.000 | 100.000 |
| TKIDN2019116 | 0.000 | 100.000 |
| TRACH2001443 | 0.000 | 34.396 |
| TRACH2001684 | 62.100 | 0.000 |
| TRACH2007834 | 1.758 | 3.734 |
| TRACH2008300 | 0.000 | 6.284 |
| TRACH3001427 | 0.000 | 3.437 |
| UTERU2002410 | 0.000 | 2.173 |
| UTERU2023175 | 5.190 | 0.000 |
| UTERU3001572 | 0.000 | 6.758 |
| BLADE2006830 | 0.000 | 6.222 |
| BRALZ2017844 | 0.000 | 50.604 |
| CTONG2028758 | 0.000 | 59.532 |
| FCBBF1000509 | 0.000 | 6.907 |
| FEBRA2001990 | 0.000 | 13.433 |
| FEBRA2028516 | 0.000 | 4.234 |
| HCHON2000508 | 0.000 | 1.382 |
| MESAN2005303 | 0.000 | 29.326 |
| NT2RI2009583 | 10.920 | 0.828 |
| TESTI2015626 | 0.000 | 4.869 |
| TKIDN2008778 | 0.000 | 100.000 |
| TKIDN2012771 | 0.000 | 100.000 |
| TKIDN2018926 | 0.000 | 78.604 |

The result of comparative analysis of cDNA libraries derived from liver tumor (TLIVE), and normal liver (LIVER) showed the following genes whose expression levels differed between the two.

TABLE 15

| Clone ID | LIVER | TLIVE |
|---|---|---|
| BRCAN2018935 | 79.072 | 0.000 |
| BRSTN2016470 | 1.897 | 0.000 |
| BRTHA2012980 | 0.000 | 86.110 |
| BRTHA3002427 | 0.000 | 19.655 |
| CTONG2028124 | 0.000 | 3.455 |
| LIVER2007415 | 100.000 | 0.000 |
| NT2RI2008724 | 0.000 | 12.626 |
| SPLEN2012624 | 43.831 | 0.000 |
| SPLEN2033098 | 0.000 | 34.211 |
| TESOP2002451 | 0.000 | 16.330 |
| TLIVE2000023 | 0.000 | 100.000 |
| TLIVE2001327 | 0.000 | 64.491 |
| TLIVE2001828 | 0.000 | 89.183 |
| TLIVE2001927 | 0.000 | 100.000 |
| TLIVE2002336 | 0.000 | 100.000 |
| TLIVE2002338 | 0.000 | 100.000 |
| TLIVE2002690 | 0.000 | 41.431 |
| TLIVE2003197 | 0.000 | 100.000 |
| TLIVE2003225 | 0.000 | 100.000 |
| TLIVE2003381 | 0.000 | 100.000 |
| TLIVE2003970 | 0.000 | 15.901 |
| TLIVE2004110 | 0.000 | 70.982 |
| TLIVE2004320 | 0.000 | 87.217 |
| TLIVE2004601 | 0.000 | 100.000 |
| TLIVE2005180 | 0.000 | 100.000 |
| TLIVE2006236 | 0.000 | 100.000 |
| TLIVE2006529 | 0.000 | 100.000 |
| TLIVE2007132 | 0.000 | 100.000 |
| TLIVE2007528 | 0.000 | 100.000 |
| TLIVE2007816 | 0.000 | 100.000 |
| TLIVE2008083 | 0.000 | 100.000 |
| TLIVE2008229 | 0.000 | 91.317 |
| TLIVE2009541 | 0.000 | 100.000 |
| UTERU2002410 | 0.000 | 4.037 |
| UTERU2005621 | 19.540 | 0.000 |
| LIVER2000247 | 100.000 | 0.000 |
| NT2RI2009583 | 1.939 | 4.615 |
| TESTI2015626 | 0.000 | 2.261 |
| TLIVE2001684 | 0.000 | 100.000 |
| TLIVE2002046 | 0.000 | 12.478 |
| TLIVE2007607 | 0.000 | 100.000 |

The result of comparative analysis of cDNA libraries derived from lung tumor (TLUNG), and normal lung (HLUNG) showed the following genes whose expression levels differed between the two.

TABLE 16

| Clone ID | HLUNG | TLUNG |
|---|---|---|
| BRCAN2021028 | 38.589 | 0.000 |
| BRHIP2000819 | 7.923 | 0.000 |
| BRSTN2016470 | 0.803 | 0.000 |
| CTONG1000087 | 3.835 | 0.000 |
| CTONG2028124 | 9.217 | 0.000 |
| HCHON2006250 | 0.000 | 32.381 |
| HEART1000074 | 2.784 | 0.000 |
| HLUNG1000017 | 100.000 | 0.000 |
| HLUNG2000014 | 100.000 | 0.000 |
| HLUNG2001996 | 76.784 | 0.000 |
| HLUNG2002465 | 4.436 | 0.000 |
| HLUNG2002958 | 100.000 | 0.000 |
| HLUNG2003003 | 29.311 | 0.000 |
| HLUNG2003872 | 100.000 | 0.000 |
| HLUNG2010464 | 100.000 | 0.000 |
| HLUNG2011041 | 100.000 | 0.000 |
| HLUNG2011298 | 35.254 | 0.000 |
| HLUNG2012049 | 100.000 | 0.000 |
| HLUNG2012287 | 100.000 | 0.000 |
| HLUNG2012727 | 100.000 | 0.000 |
| HLUNG2013204 | 100.000 | 0.000 |
| HLUNG2013304 | 100.000 | 0.000 |
| HLUNG2013622 | 100.000 | 0.000 |
| HLUNG2013851 | 100.000 | 0.000 |
| HLUNG2014262 | 100.000 | 0.000 |
| HLUNG2014288 | 100.000 | 0.000 |
| HLUNG2014449 | 100.000 | 0.000 |
| HLUNG2015617 | 100.000 | 0.000 |
| HLUNG2017350 | 100.000 | 0.000 |
| HLUNG2017546 | 12.944 | 0.000 |
| HLUNG2017806 | 100.000 | 0.000 |
| HLUNG2019058 | 100.000 | 0.000 |
| HSYRA2008376 | 11.470 | 0.000 |
| KIDNE2012945 | 51.268 | 0.000 |
| NT2RI2003993 | 13.924 | 0.000 |
| NT2RP7013795 | 0.000 | 89.568 |
| OCBBF3000483 | 14.638 | 0.000 |
| SPLEN2028914 | 10.242 | 0.000 |
| SPLEN2031547 | 5.775 | 0.000 |
| SYNOV4007671 | 2.689 | 0.000 |
| TESOP1000127 | 34.899 | 0.000 |
| TESTI2003573 | 27.394 | 0.000 |
| TESTI4000014 | 0.655 | 0.000 |
| TESTI4037156 | 2.030 | 0.000 |
| TRACH2005811 | 3.868 | 0.000 |
| TRACH3004068 | 5.227 | 0.000 |
| UTERU2005621 | 8.268 | 0.000 |
| FEBRA2028516 | 4.195 | 0.000 |
| HCHON2000508 | 1.370 | 0.000 |
| HLUNG2013350 | 100.000 | 0.000 |
| HLUNG2015418 | 76.605 | 0.000 |
| HLUNG2015548 | 100.000 | 0.000 |
| HLUNG2016862 | 100.000 | 0.000 |
| NT2RI2009583 | 0.000 | 13.890 |
| TESTI2015626 | 2.412 | 0.000 |
| TRACH2019672 | 76.605 | 0.000 |

The result of comparative analysis of cDNA libraries derived from ovary tumor (TOVER), and normal ovary (NOVER) showed the following genes whose expression levels differed between the two.

TABLE 17A

| Clone ID | NOVAR | TOVAR |
|---|---|---|
| CTONG2019788 | 72.878 | 0.000 |
| FEBRA2014213 | 0.000 | 85.773 |
| HLUNG2017546 | 84.114 | 0.000 |
| NOVAR2000136 | 100.000 | 0.000 |
| NOVAR2000710 | 83.961 | 0.000 |
| NOVAR2000962 | 100.000 | 0.000 |
| NOVAR2001108 | 100.000 | 0.000 |
| NOVAR2001783 | 95.973 | 0.000 |
| OCBBF3007516 | 90.145 | 0.000 |
| TESTI2052693 | 39.903 | 0.000 |
| TOVAR2000649 | 0.000 | 100.000 |
| TOVAR2001281 | 0.000 | 100.000 |
| TOVAR2001730 | 0.000 | 100.000 |
| TOVAR2002247 | 0.000 | 100.000 |
| TOVAR2002549 | 0.000 | 100.000 |
| TRACH3004068 | 0.000 | 31.044 |

The result of comparative analysis of cDNA libraries derived from ovary tumor (TOVER), and normal ovary (NOVER) showed the following genes whose expression levels differed between the two.

TABLE 17B

| Clone ID | NOVAR | TOVAR |
|---|---|---|
| TESTI2015626 | 7.838 | 7.163 |

The gene has no different expression levels between normal and diseased ovary. However, the gene showed significantly different expression level in both ovary tumor and normal ovary, compared with other tissues (as in Example 9). Thus, the gene are ovary-specific gene and can be used as diagnostic marker because its association with the disease.

The result of comparative analysis of cDNA libraries derived from stomach tumor (TSTOM) and normal stomach (STOMA) showed the following genes whose expression levels differed between the two.

TABLE 18

| Clone ID | STOMA | TSTOM |
|---|---|---|
| BRACE2024627 | 83.309 | 0.000 |
| BRAWH2014645 | 0.000 | 22.702 |
| BRCAN2028355 | 0.000 | 31.728 |
| BRHIP2000819 | 14.701 | 46.392 |
| BRSTN2016470 | 1.490 | 0.000 |
| BRTHA3003490 | 0.000 | 34.864 |
| COLON2002443 | 30.687 | 0.000 |
| HEART1000010 | 23.250 | 0.000 |
| HLUNG2002465 | 8.231 | 0.000 |
| KIDNE2001847 | 0.000 | 78.156 |
| NT2RP7000466 | 5.924 | 0.000 |
| PUAEN2006328 | 0.000 | 79.193 |
| SMINT2001818 | 12.758 | 0.000 |
| STOMA1000189 | 10.642 | 0.000 |
| STOMA2003444 | 91.236 | 0.000 |
| STOMA2004294 | 100.000 | 0.000 |
| STOMA2004925 | 85.088 | 0.000 |
| STOMA2008546 | 100.000 | 0.000 |
| SYNOV4007671 | 4.989 | 0.000 |
| TESTI4000014 | 0.000 | 3.835 |
| TESTI4010851 | 3.720 | 0.000 |
| THYMU2035735 | 0.000 | 28.332 |
| TRACH2001549 | 28.775 | 0.000 |
| TRACH2005811 | 0.000 | 22.648 |
| TRACH2025535 | 0.000 | 12.468 |
| TSTOM1000135 | 0.000 | 90.639 |
| TSTOM2000442 | 0.000 | 100.000 |
| TSTOM2000553 | 0.000 | 36.203 |
| TSTOM2002672 | 0.000 | 100.000 |
| UTERU2006115 | 53.335 | 0.000 |
| UTERU3001572 | 12.423 | 0.000 |
| FEBRA2008692 | 0.000 | 52.692 |
| NT2RI2009583 | 7.613 | 0.000 |
| STOMA2003158 | 41.655 | 0.000 |
| STOMA2004893 | 49.171 | 0.000 |
| TESTI2015626 | 2.238 | 0.000 |

The result of comparative analysis of cDNA libraries derived from uterine tumor (TUTER) and normal uterus (UTERU) showed the following genes whose expression levels differed between the two.

TABLE 19

| Clone ID | UTERU | TUTER |
|---|---|---|
| BNGH42007788 | 3.672 | 0.000 |
| BRACE1000186 | 2.579 | 0.000 |
| BRACE2030341 | 16.499 | 0.000 |
| BRACE3008772 | 62.692 | 0.000 |
| BRACE3009747 | 2.668 | 0.000 |
| BRACE3010428 | 5.461 | 0.000 |
| BRACE3027478 | 19.089 | 0.000 |
| BRALZ2017359 | 24.816 | 0.000 |
| BRAWH2014645 | 2.522 | 0.000 |
| BRAWH3000314 | 28.447 | 0.000 |
| BRAWH3001326 | 54.394 | 0.000 |
| BRAWH3002574 | 7.885 | 0.000 |
| BRAWH3002821 | 26.183 | 0.000 |
| BRAWH3003727 | 11.993 | 0.000 |
| BRAWH3007592 | 4.277 | 0.000 |
| BRCAN2009432 | 3.141 | 29.345 |
| BRCAN2028355 | 1.762 | 0.000 |
| BRHIP3007586 | 4.116 | 0.000 |
| BRHIP3008344 | 53.896 | 0.000 |
| BRHIP3008565 | 53.896 | 0.000 |
| BRSSN2006892 | 18.468 | 0.000 |
| BRSTN2001067 | 7.384 | 0.000 |
| BRSTN2016470 | 0.522 | 0.000 |
| BRTHA2010608 | 51.819 | 0.000 |
| BRTHA3003074 | 13.429 | 0.000 |
| CTONG1000087 | 2.494 | 0.000 |
| CTONG1000467 | 10.248 | 0.000 |
| CTONG2028124 | 2.997 | 0.000 |
| CTONG3001123 | 9.359 | 0.000 |
| CTONG3008894 | 0.867 | 0.000 |
| CTONG3009028 | 3.839 | 0.000 |
| CTONG3009239 | 1.722 | 0.000 |
| FCBBF3004847 | 39.231 | 0.000 |
| FEBRA2026984 | 20.914 | 0.000 |
| FEBRA2028618 | 6.061 | 0.000 |
| HCHON2000244 | 1.569 | 0.000 |
| HCHON2000418 | 15.860 | 0.000 |
| HCHON2000626 | 5.608 | 0.000 |
| HCHON2001084 | 2.739 | 0.000 |
| HCHON2001217 | 4.966 | 0.000 |
| HCHON2005921 | 24.732 | 0.000 |
| HCHON2006250 | 0.000 | 34.872 |
| HCHON2008444 | 15.860 | 0.000 |
| HLUNG2003003 | 28.594 | 0.000 |
| HSYRA2008376 | 1.865 | 0.000 |
| KIDNE2002252 | 2.600 | 0.000 |
| MESAN2014295 | 24.337 | 0.000 |
| NOVAR2000710 | 4.201 | 0.000 |
| NT2RI2008724 | 2.190 | 0.000 |
| NT2RI2014247 | 18.191 | 0.000 |
| NT2RI2014733 | 29.743 | 0.000 |
| NT2RI3002892 | 18.890 | 0.000 |
| NT2RI3005724 | 7.069 | 0.000 |
| NT2RI3006284 | 0.000 | 46.111 |
| NT2RI3006340 | 19.479 | 0.000 |
| NT2RI3006673 | 11.902 | 0.000 |
| NT2RI3007291 | 14.928 | 0.000 |
| NT2RI3007543 | 1.012 | 0.000 |
| NT2RP7004123 | 2.289 | 0.000 |
| NT2RP7005529 | 8. [symbol] 06 | 0.000 |
| NT2RP7009147 | 1.900 | 0.000 |
| NT2RP7017474 | 22.450 | 0.000 |
| OCBBF2007028 | 8.234 | 0.000 |
| OCBBF2020741 | 33.655 | 0.000 |
| OCBBF2024850 | 26.505 | 0.000 |
| OCBBF2036743 | 13.183 | 0.000 |
| OCBBF3000483 | 9.520 | 0.000 |
| PLACE6001185 | 25.473 | 0.000 |
| PLACE7000514 | 5.673 | 0.000 |
| PUAEN2007044 | 1.710 | 0.000 |
| PUAEN2009655 | 4.359 | 0.000 |
| SKNSH2000482 | 23.251 | 0.000 |
| SPLEN2006122 | 0.000 | 18.769 |
| SPLEN2016554 | 22.228 | 0.000 |
| SPLEN2031547 | 5.633 | 0.000 |
| SPLEN2036932 | 3.497 | 0.000 |
| STOMA1000189 | 1.865 | 0.000 |
| STOMA2004925 | 14.912 | 0.000 |
| SYNOV2017055 | 20.136 | 0.000 |
| SYNOV4001395 | 21.660 | 0.000 |
| SYNOV4002346 | 7.153 | 0.000 |
| SYNOV4008440 | 1.759 | 0.000 |
| TCERX2000613 | 14.497 | 0.000 |
| TESOP2002273 | 4.841 | 0.000 |
| TESTI4000014 | 0.639 | 0.000 |
| TESTI4008797 | 14.824 | 0.000 |
| TESTI4009286 | 2.594 | 0.000 |
| TESTI4012702 | 2.600 | 0.000 |
| TESTI4013675 | 35.326 | 0.000 |
| TESTI4014159 | 13.979 | 0.000 |
| TESTI4018886 | 64.596 | 0.000 |
| TESTI4029671 | 22.183 | 0.000 |
| TESTI4037156 | 1.320 | 0.000 |
| THYMU2008725 | 15.552 | 0.000 |
| THYMU2031890 | 21.176 | 0.000 |
| THYMU2033070 | 58.853 | 0.000 |
| THYMU2035735 | 11.014 | 0.000 |
| THYMU3001472 | 20.097 | 0.000 |
| TRACH1000205 | 4.694 | 0.000 |
| TRACH2001443 | 11.083 | 0.000 |
| TRACH2001549 | 5.043 | 0.000 |
| TRACH2005811 | 1.258 | 0.000 |
| TRACH2007834 | 0.602 | 0.000 |
| TRACH2008300 | 2.025 | 0.000 |
| TRACH3002192 | 4.463 | 0.000 |
| TRACH3003379 | 29.185 | 0.000 |
| TRACH3004068 | 1.700 | 0.000 |
| TRACH3004721 | 3.467 | 0.000 |
| TRACH3007479 | 3.848 | 0.000 |
| TUTER1000122 | 0.000 | 72.738 |
| TUTER2000425 | 0.000 | 100.000 |
| TUTER2000904 | 3.330 | 62.217 |
| TUTER2000916 | 0.000 | 100.000 |
| TUTER2001387 | 0.000 | 100.000 |
| TUTER2002729 | 0.000 | 100.000 |
| UTERU1000024 | 100.000 | 0.000 |
| UTERU1000031 | 100.000 | 0.000 |
| UTERU1000148 | 100.000 | 0.000 |
| UTERU1000249 | 100.000 | 0.000 |
| UTERU1000337 | 100.000 | 0.000 |
| UTERU1000339 | 100.000 | 0.000 |
| UTERU2000649 | 100.000 | 0.000 |
| UTERU2001409 | 100.000 | 0.000 |
| UTERU2002410 | 0.700 | 13.085 |
| UTERU2002841 | 64.596 | 0.000 |
| UTERU2004688 | 6.630 | 0.000 |
| UTERU2004929 | 13.845 | 0.000 |
| UTERU2005004 | 41.697 | 0.000 |
| UTERU2005621 | 5.377 | 0.000 |
| UTERU2006115 | 9.347 | 0.000 |
| UTERU2006137 | 9.269 | 0.000 |
| UTERU2006568 | 100.000 | 0.000 |
| UTERU2007444 | 64.596 | 0.000 |
| UTERU2007520 | 15.999 | 0.000 |
| UTERU2007724 | 15.672 | 0.000 |
| UTERU2008347 | 100.000 | 0.000 |
| UTERU2014678 | 48.981 | 0.000 |
| UTERU2017762 | 39.037 | 0.000 |
| UTERU2019491 | 100.000 | 0.000 |
| UTERU2019681 | 100.000 | 0.000 |
| UTERU2019706 | 54.394 | 0.000 |
| UTERU2019940 | 51.819 | 0.000 |
| UTERU2020491 | 100.000 | 0.000 |
| UTERU2020718 | 100.000 | 0.000 |
| UTERU2021163 | 53.896 | 0.000 |
| UTERU2021380 | 100.000 | 0.000 |
| UTERU2022020 | 100.000 | 0.000 |
| UTERU2022981 | 100.000 | 0.000 |
| UTERU2023039 | 54.394 | 0.000 |
| UTERU2023175 | 7.103 | 0.000 |
| UTERU2023651 | 29.963 | 0.000 |
| UTERU2023712 | 100.000 | 0.000 |
| UTERU2024002 | 100.000 | 0.000 |
| UTERU2024656 | 51.568 | 0.000 |
| UTERU2025025 | 100.000 | 0.000 |
| UTERU2025645 | 100.000 | 0.000 |
| UTERU2025891 | 100.000 | 0.000 |
| UTERU2026025 | 100.000 | 0.000 |
| UTERU2026090 | 100.000 | 0.000 |
| UTERU2026203 | 54.394 | 0.000 |
| UTERU2027591 | 100.000 | 0.000 |
| UTERU2029953 | 100.000 | 0.000 |
| UTERU2030213 | 58.763 | 0.000 |
| UTERU2030280 | 51.819 | 0.000 |
| UTERU2031084 | 25.928 | 0.000 |
| UTERU2031268 | 100.000 | 0.000 |
| UTERU2031521 | 100.000 | 0.000 |
| UTERU2031703 | 100.000 | 0.000 |
| UTERU2031851 | 100.000 | 0.000 |
| UTERU2033375 | 1.385 | 0.000 |
| UTERU2033382 | 100.000 | 0.000 |
| UTERU2035114 | 51.568 | 0.000 |
| UTERU2035323 | 100.000 | 0.000 |
| UTERU2035328 | 100.000 | 0.000 |
| UTERU2035331 | 100.000 | 0.000 |
| UTERU2035452 | 100.000 | 0.000 |
| UTERU2035469 | 100.000 | 0.000 |
| UTERU2035503 | 100.000 | 0.000 |
| UTERU2035745 | 100.000 | 0.000 |
| UTERU2036089 | 100.000 | 0.000 |
| UTERU2037361 | 100.000 | 0.000 |
| UTERU2037577 | 100.000 | 0.000 |
| UTERU2038251 | 100.000 | 0.000 |
| UTERU3000226 | 62.692 | 0.000 |
| UTERU3000645 | 34.742 | 0.000 |
| UTERU3000665 | 100.000 | 0.000 |
| UTERU3000828 | 100.000 | 0.000 |
| UTERU3000899 | 33.654 | 0.000 |
| UTERU3001059 | 100.000 | 0.000 |
| UTERU3001240 | 100.000 | 0.000 |
| UTERU3001542 | 58.853 | 0.000 |
| UTERU3001571 | 51.819 | 0.000 |
| UTERU3001572 | 19.596 | 0.000 |
| UTERU3001585 | 100.000 | 0.000 |
| UTERU3001652 | 100.000 | 0.000 |
| UTERU3001766 | 54.206 | 0.000 |
| UTERU3001988 | 100.000 | 0.000 |
| UTERU3002209 | 100.000 | 0.000 |
| UTERU3002218 | 51.568 | 0.000 |
| UTERU3002383 | 100.000 | 0.000 |
| UTERU3002667 | 100.000 | 0.000 |
| UTERU3002731 | 100.000 | 0.000 |
| UTERU3002768 | 100.000 | 0.000 |
| UTERU3002786 | 51.568 | 0.000 |
| UTERU3002993 | 100.000 | 0.000 |
| UTERU3003116 | 100.000 | 0.000 |
| UTERU3003135 | 39.146 | 0.000 |
| UTERU3003178 | 100.000 | 0.000 |
| UTERU3003465 | 100.000 | 0.000 |
| UTERU3003523 | 100.000 | 0.000 |
| UTERU3003776 | 100.000 | 0.000 |
| UTERU3004523 | 100.000 | 0.000 |
| UTERU3004616 | 100.000 | 0.000 |
| UTERU3004709 | 51.819 | 0.000 |
| UTERU3004992 | 100.000 | 0.000 |
| UTERU3005049 | 58.853 | 0.000 |
| UTERU3005205 | 100.000 | 0.000 |
| UTERU3005230 | 58.249 | 0.000 |
| UTERU3005460 | 62.692 | 0.000 |
| UTERU3005585 | 100.000 | 0.000 |
| UTERU3005907 | 15.610 | 0.000 |
| UTERU3005970 | 62.692 | 0.000 |
| UTERU3006008 | 100.000 | 0.000 |
| UTERU3006308 | 62.692 | 0.000 |
| UTERU3007134 | 100.000 | 0.000 |
| UTERU3007419 | 62.692 | 0.000 |
| UTERU3007640 | 54.394 | 0.000 |
| UTERU3007913 | 87.049 | 0.000 |
| UTERU3008660 | 100.000 | 0.000 |
| UTERU3008671 | 100.000 | 0.000 |
| UTERU3009259 | 53.896 | 0.000 |
| UTERU3009490 | 100.000 | 0.000 |
| UTERU3009517 | 54.206 | 0.000 |
| UTERU3009690 | 100.000 | 0.000 |
| UTERU3009871 | 21.605 | 0.000 |
| UTERU3009979 | 100.000 | 0.000 |
| UTERU3011063 | 100.000 | 0.000 |
| UTERU3015086 | 100.000 | 0.000 |
| UTERU3015500 | 100.000 | 0.000 |
| UTERU3016789 | 100.000 | 0.000 |
| UTERU3018081 | 40.205 | 0.000 |
| UTERU3018154 | 100.000 | 0.000 |
| UTERU3018616 | 25.167 | 0.000 |
| UTERU3018711 | 86.466 | 0.000 |
| ADRGL2000042 | 3.029 | 0.000 |
| BRHIP3000017 | 15.777 | 0.000 |
| CTONG2003348 | 39.037 | 0.000 |
| CTONG2019822 | 7.755 | 0.000 |
| CTONG2020378 | 19.250 | 0.000 |
| CTONG2020411 | 32.844 | 0.000 |
| CTONG2024031 | 6.165 | 0.000 |
| FEBRA2028516 | 1.364 | 0.000 |
| HCASM2008536 | 15.228 | 0.000 |
| HCHON2000743 | 7.282 | 0.000 |
| IMR322001879 | 11.495 | 0.000 |
| MESAN2005303 | 9.449 | 0.000 |
| NT2RI2009583 | 0.267 | 0.000 |
| OCBBF2008144 | 3.440 | 0.000 |
| PERIC2007068 | 5.521 | 0.000 |
| SPLEN2039379 | 10.362 | 0.000 |
| TESTI2015626 | 0.784 | 7.329 |
| TESTI4013894 | 33.408 | 0.000 |
| TUTER2000057 | 0.000 | 92.461 |
| UTERU2004299 | 100.000 | 0.000 |
| UTERU2008040 | 14.037 | 0.000 |
| UTERU2011220 | 4.669 | 0.000 |
| UTERU2019534 | 100.000 | 0.000 |
| UTERU2021820 | 29.732 | 0.000 |
| UTERU2028734 | 26.183 | 0.000 |
| UTERU2032279 | 100.000 | 0.000 |
| UTERU2033577 | 100.000 | 0.000 |
| UTERU2035978 | 100.000 | 0.000 |
| UTERU3000402 | 100.000 | 0.000 |
| UTERU3000738 | 41.697 | 0.000 |
| UTERU3001053 | 100.000 | 0.000 |
| UTERU3014791 | 100.000 | 0.000 |
| UTERU3015412 | 100.000 | 0.000 |
| UTERU3017176 | 100.000 | 0.000 |

The result of comparative analysis of cDNA libraries derived from tongue cancer (CTONG) and normal tongue (NTONG) showed the following genes whose expression levels differed between the two.

TABLE 20

| Clone ID | NTONG | CTONG |
|---|---|---|
| BNGH42007788 | 0.000 | 5.734 |
| BRACE1000186 | 15.935 | 0.000 |
| BRACE2006319 | 0.000 | 6.530 |
| BRACE3010428 | 0.000 | 6.396 |
| BRACE3012364 | 0.000 | 7.922 |
| BRAMY2020058 | 0.000 | 52.028 |
| BRAMY3002803 | 0.000 | 26.873 |
| BRAWH2001671 | 0.000 | 7.082 |
| BRAWH2014645 | 0.000 | 1.969 |
| BRAWH3002574 | 0.000 | 12.314 |
| BRCAN2009432 | 0.000 | 4.905 |
| BRCAN2015371 | 0.000 | 20.417 |
| BRCAN2020710 | 0.000 | 20.242 |
| BRHIP2004814 | 0.000 | 64.609 |
| BRHIP3018797 | 0.000 | 2.495 |
| BRTHA2003461 | 0.000 | 4.088 |
| BRTHA3003490 | 11.964 | 0.000 |
| CTONG1000087 | 0.000 | 3.895 |
| CTONG1000088 | 0.000 | 5.442 |
| CTONG1000288 | 11.209 | 84.986 |
| CTONG1000302 | 0.000 | 100.000 |
| CTONG1000341 | 0.000 | 51.706 |
| CTONG1000467 | 0.000 | 16.004 |
| CTONG1000488 | 0.000 | 100.000 |
| CTONG1000508 | 0.000 | 100.000 |
| CTONG1000540 | 0.000 | 100.000 |
| CTONG2000042 | 0.000 | 65.252 |
| CTONG2001877 | 0.000 | 100.000 |
| CTONG2004062 | 0.000 | 100.000 |
| CTONG2006798 | 0.000 | 17.972 |
| CTONG2008233 | 0.000 | 8.953 |
| CTONG2009423 | 0.000 | 64.609 |
| CTONG2009531 | 0.000 | 100.000 |
| CTONG2010803 | 0.000 | 20.971 |
| CTONG2013178 | 0.000 | 35.252 |
| CTONG2017500 | 0.000 | 4.934 |
| CTONG2019248 | 0.000 | 28.179 |
| CTONG2019652 | 0.000 | 100.000 |
| CTONG2019704 | 0.000 | 62.446 |
| CTONG2019788 | 0.000 | 11.390 |
| CTONG2019833 | 0.000 | 100.000 |
| CTONG2020127 | 0.000 | 100.000 |
| CTONG2020522 | 0.000 | 42.683 |
| CTONG2020638 | 0.000 | 23.060 |
| CTONG2020806 | 0.000 | 100.000 |
| CTONG2021132 | 0.000 | 100.000 |
| CTONG2022153 | 0.000 | 100.000 |
| CTONG2022601 | 0.000 | 100.000 |
| CTONG2023021 | 0.000 | 62.680 |
| CTONG2023512 | 0.000 | 100.000 |
| CTONG2024206 | 0.000 | 100.000 |
| CTONG2024749 | 0.000 | 100.000 |
| CTONG2025496 | 0.000 | 100.000 |
| CTONG2025516 | 0.000 | 100.000 |
| CTONG2025900 | 0.000 | 100.000 |
| CTONG2026920 | 0.000 | 100.000 |
| CTONG2027327 | 0.000 | 52.760 |
| CTONG2028124 | 3.704 | 0.936 |
| CTONG2028687 | 0.000 | 100.000 |
| CTONG30000 [symbol] 4 | 0.000 | 51.585 |
| CTONG3000657 | 0.000 | 7.226 |
| CTONG3000686 | 0.000 | 100.000 |
| CTONG3000707 | 0.000 | 100.000 |
| CTONG3000896 | 0.000 | 100.000 |
| CTONG3001123 | 0.000 | 14.616 |
| CTONG3001370 | 0.000 | 65.252 |
| CTONG3001420 | 0.000 | 51.138 |
| CTONG3001560 | 0.000 | 100.000 |
| CTONG3002020 | 0.000 | 100.000 |
| CTONG3002127 | 0.000 | 62.446 |
| CTONG3002412 | 0.000 | 19.932 |
| CTONG3002674 | 0.000 | 41.611 |
| CTONG3003179 | 0.000 | 100.000 |
| CTONG3003483 | 0.000 | 100.000 |
| CTONG3003652 | 0.000 | 100.000 |
| CTONG3003654 | 0.000 | 100.000 |
| CTONG3003737 | 0.000 | 100.000 |
| CTONG3003905 | 0.000 | 36.474 |
| CTONG3003972 | 0.000 | 51.706 |
| CTONG3004072 | 0.000 | 36.356 |
| CTONG3004712 | 0.000 | 100.000 |
| CTONG3005325 | 0.000 | 100.000 |
| CTONG3005648 | 0.000 | 100.000 |
| CTONG3005713 | 0.000 | 100.000 |
| CTONG3005813 | 0.000 | 72.408 |
| CTONG3006067 | 0.000 | 74.021 |
| CTONG3006186 | 0.000 | 100.000 |
| CTONG3006650 | 0.000 | 100.000 |
| CTONG3007444 | 0.000 | 100.000 |
| CTONG3007528 | 0.000 | 100.000 |
| CTONG3007586 | 0.000 | 100.000 |
| CTONG3007870 | 0.000 | 100.000 |
| CTONG3008252 | 0.000 | 100.000 |
| CTONG3008258 | 0.000 | 100.000 |
| CTONG3008496 | 0.000 | 100.000 |
| CTONG3008566 | 0.000 | 100.000 |
| CTONG3008639 | 0.000 | 100.000 |
| CTONG3008831 | 0.000 | 100.000 |
| CTONG3008894 | 0.000 | 2.708 |
| CTONG3008951 | 0.000 | 100.000 |
| CTONG3009028 | 0.000 | 5.995 |
| CTONG3009227 | 0.000 | 100.000 |
| CTONG3009239 | 0.000 | 5.378 |
| CTONG3009328 | 0.000 | 44.674 |
| CTONG3009385 | 0.000 | 100.000 |
| FEBRA2007544 | 0.000 | 4.560 |
| FEBRA2007801 | 0.000 | 4.598 |
| FEBRA2021966 | 31.791 | 0.000 |
| FEBRA2025427 | 0.000 | 9.234 |
| HCHON2000028 | 0.000 | 2.955 |
| HCHON2001217 | 0.000 | 3.877 |
| HHDPC1000118 | 0.000 | 3.904 |
| HSYRA2008376 | 11.524 | 0.000 |
| KIDNE2001847 | 0.000 | 6.778 |
| KIDNE2002252 | 0.000 | 4.060 |
| MESAN2006563 | 0.000 | 2.572 |
| NT2RI2008724 | 0.000 | 3.421 |
| NT2RI2018883 | 0.000 | 50.616 |
| NT2RI3000622 | 63.099 | 0.000 |
| NT2RI3006284 | 0.000 | 3.854 |
| NT2RI3006673 | 0.000 | 18.588 |
| NT2RI3007543 | 6.253 | 7.901 |
| NT2RI3007757 | 0.000 | 23.555 |
| NT2RP7004123 | 0.000 | 3.574 |
| NT2RP7009147 | 0.000 | 2.967 |
| NT2RP7014005 | 0.000 | 10.836 |
| NTONG2000413 | 79.538 | 0.000 |
| NTONG2003852 | 49.507 | 0.000 |
| NTONG2005277 | 100.000 | 0.000 |
| NTONG2005969 | 23.675 | 0.000 |
| NTONG2006354 | 100.000 | 0.000 |
| NTONG2007249 | 100.000 | 0.000 |
| NTONG2007517 | 100.000 | 0.000 |
| NTONG2008088 | 75.309 | 0.000 |
| NTONG2008672 | 100.000 | 0.000 |
| OCBBF2001794 | 34.843 | 0.000 |
| OCBBF2006151 | 0.000 | 10.738 |
| PEBLM2004666 | 0.000 | 6.920 |
| PEBLM2005183 | 0.000 | 7.010 |
| SPLEN2002467 | 0.000 | 6.068 |
| SPLEN2029912 | 0.000 | 6.085 |
| SPLEN2031547 | 0.000 | 2.932 |
| SYNOV4007671 | 0.000 | 1.365 |
| SYNOV4008440 | 0.000 | 2.748 |
| TBAES2002197 | 0.000 | 28.875 |
| TESOP2002273 | 0.000 | 7.560 |
| TESTI2009474 | 0.000 | 3.763 |
| TESTI4000014 | 2.632 | 0.665 |
| TESTI4000209 | 0.000 | 4.934 |
| TESTI4008018 | 0.000 | 74.021 |
| TESTI4009286 | 0.000 | 1.350 |
| TESTI4010851 | 0.000 | 1.018 |
| TESTI4012702 | 0.000 | 4.060 |
| TESTI4013675 | 0.000 | 18.389 |
| THYMU2031847 | 0.000 | 69.076 |
| THYMU2033308 | 0.000 | 26.010 |
| TLIVE2002690 | 44.414 | 0.000 |
| TRACH2005811 | 0.000 | 3.928 |
| TRACH2007059 | 0.000 | 62.446 |
| TRACH2025535 | 0.000 | 2.163 |
| TRACH3001427 | 0.000 | 1.729 |
| TSTOM2000553 | 0.000 | 12.559 |
| UTERU2005621 | 0.000 | 8.397 |
| UTERU2017762 | 0.000 | 60.963 |
| UTERU2023175 | 0.000 | 2.773 |
| UTERU3001572 | 0.000 | 6.800 |
| BLADE2006830 | 24.778 | 0.000 |
| BRHIP3000017 | 0.000 | 8.213 |
| CTONG1000113 | 0.000 | 20.967 |
| CTONG2003348 | 0.000 | 60.963 |
| CTONG2004000 | 0.000 | 100.000 |
| CTONG2008721 | 0.000 | 62.680 |
| CTONG2015596 | 0.000 | 100.000 |
| CTONG2015633 | 0.000 | 100.000 |
| CTONG2016942 | 0.000 | 21.398 |
| CTONG2019822 | 0.000 | 12.111 |
| CTONG2020374 | 0.000 | 57.889 |
| CTONG2020378 | 0.000 | 30.063 |
| CTONG2020411 | 0.000 | 12.823 |
| CTONG2020974 | 0.000 | 74.021 |
| CTONG2024031 | 0.000 | 19.255 |
| CTONG2028758 | 0.000 | 29.955 |
| CTONG3001501 | 0.000 | 100.000 |
| CTONG3002552 | 0.000 | 100.000 |
| CTONG3003598 | 0.000 | 100.000 |
| CTONG3004550 | 0.000 | 100.000 |
| CTONG3004726 | 0.000 | 31.543 |
| CTONG3009287 | 0.000 | 100.000 |
| FEBRA2008692 | 0.000 | 13.709 |
| FEBRA2028516 | 0.000 | 4.261 |
| HCHON2000508 | 2.752 | 9.042 |
| NT2RI2009583 | 4.947 | 0.833 |
| NTONG2008093 | 100.000 | 0.000 |
| PERIC2007068 | 0.000 | 2.874 |
| TESOP2007384 | 44.382 | 37.388 |
| TLIVE2002046 | 0.000 | 3.380 |
| TRACH2000862 | 0.000 | 53.910 |

The result of comparative analysis of cDNA libraries derived from fetal brain (FCBBF, FEBRA, or OCBBF) and adult brain (BRACE, BRALZ, BRAMY, BRAWH, BRCAN, BRCOC, BRHIP, BRSSN, BRSTN, or BRTHA) showed the following genes whose expression levels differed between the two.

TABLE 21

| Clone ID | FCBBF | FEBRA | OCBBF | BRACE | BRALZ | BRAMY | BRAWH | BRCAN | BRCOC | BRHIP | BRSSN | BRSTN | BRTHA |
|---|---|---|---|---|---|---|---|---|---|---|---|---|---|
| ADRGL2009146 | 0.000 | 0.000 | 0.000 | 10.913 | 0.000 | 0.000 | 0.000 | 0.000 | 0.000 | 0.000 | 0.000 | 0.000 | 0.000 |
| ADRGL2012038 | 0.000 | 0.000 | 0.000 | 1.459 | 0.000 | 0.000 | 0.000 | 0.000 | 0.000 | 0.000 | 0.000 | 0.000 | 0.000 |
| ADRGL2012179 | 0.000 | 0.000 | 17.655 | 0.000 | 0.000 | 0.000 | 0.000 | 0.000 | 0.000 | 0.000 | 0.000 | 0.000 | 0.000 |
| ASTRO1000009 | 0.000 | 0.000 | 0.000 | 0.000 | 0.000 | 0.000 | 2.611 | 0.000 | 0.000 | 0.000 | 0.000 | 0.000 | 0.000 |
| ASTRO2003960 | 0.000 | 0.000 | 0.000 | 17.003 | 0.000 | 0.000 | 0.000 | 0.000 | 0.000 | 0.000 | 0.000 | 0.000 | 0.000 |
| ASTRO3000482 | 0.000 | 0.000 | 0.000 | 0.000 | 0.000 | 0.000 | 0.000 | 0.000 | 0.000 | 0.000 | 0.000 | 0.000 | 24.247 |
| BLADE1000176 | 0.000 | 0.000 | 0.000 | 16.822 | 0.000 | 0.000 | 0.000 | 0.000 | 0.000 | 0.000 | 0.000 | 0.000 | 0.000 |
| BLADE2001371 | 0.000 | 0.000 | 0.000 | 0.000 | 0.000 | 0.000 | 0.000 | 0.000 | 0.000 | 12.691 | 0.000 | 0.000 | 0.000 |
| BLADE2004089 | 0.000 | 0.000 | 12.311 | 7.034 | 0.000 | 0.000 | 0.000 | 0.000 | 0.000 | 0.000 | 0.000 | 0.000 | 0.000 |
| BLADE2008398 | 0.000 | 0.000 | 0.000 | 4.401 | 0.000 | 0.000 | 12.401 | 0.000 | 0.000 | 18.978 | 0.000 | 0.000 | 13.752 |
| BNGH42007788 | 5.687 | 0.000 | 0.000 | 0.000 | 0.000 | 0.000 | 0.000 | 0.000 | 0.000 | 3.141 | 0.000 | 0.000 | 0.000 |
| BRACE1000186 | 0.000 | 0.000 | 0.000 | 1.535 | 0.000 | 0.000 | 4.324 | 0.000 | 0.000 | 2.206 | 0.000 | 7.796 | 0.000 |
| BRACE1000258 | 0.000 | 0.000 | 0.000 | 68.044 | 0.000 | 0.000 | 31.956 | 0.000 | 0.000 | 0.000 | 0.000 | 0.000 | 0.000 |
| BRACE1000533 | 8.716 | 11.762 | 11.722 | 11.720 | 0.000 | 4.754 | 11.795 | 0.000 | 0.000 | 9.627 | 8.780 | 0.000 | 5.232 |
| BRACE1000572 | 0.000 | 0.000 | 0.000 | 100.000 | 0.000 | 0.000 | 0.000 | 0.000 | 0.000 | 0.000 | 0.000 | 0.000 | 0.000 |
| BRACE2003639 | 0.000 | 0.000 | 0.000 | 100.000 | 0.000 | 0.000 | 0.000 | 0.000 | 0.000 | 0.000 | 0.000 | 0.000 | 0.000 |
| BRACE2005457 | 0.000 | 0.000 | 0.000 | 41.512 | 0.000 | 0.000 | 58.488 | 0.000 | 0.000 | 0.000 | 0.000 | 0.000 | 0.000 |
| BRACE2006319 | 6.477 | 17.479 | 8.711 | 4.977 | 0.000 | 3.532 | 0.000 | 0.000 | 0.000 | 0.000 | 0.000 | 0.000 | 0.000 |
| BRACE2008594 | 0.000 | 0.000 | 0.000 | 100.000 | 0.000 | 0.000 | 0.000 | 0.000 | 0.000 | 0.000 | 0.000 | 0.000 | 0.000 |
| BRACE2010489 | 0.000 | 0.000 | 0.000 | 15.026 | 0.000 | 10.665 | 63.510 | 0.000 | 0.000 | 10.799 | 0.000 | 0.000 | 0.000 |
| BRACE2011747 | 0.000 | 0.000 | 0.000 | 100.000 | 0.000 | 0.000 | 0.000 | 0.000 | 0.000 | 0.000 | 0.000 | 0.000 | 0.000 |
| BRACE2014306 | 0.000 | 0.000 | 0.000 | 6.909 | 0.000 | 0.000 | 0.000 | 0.000 | 34.486 | 0.000 | 0.000 | 17.550 | 5.398 |
| BRACE2014475 | 0.000 | 0.000 | 0.000 | 100.000 | 0.000 | 0.000 | 0.000 | 0.000 | 0.000 | 0.000 | 0.000 | 0.000 | 0.000 |
| BRACE2014657 | 0.000 | 0.000 | 0.000 | 76.765 | 0.000 | 7.784 | 15.451 | 0.000 | 0.000 | 0.000 | 0.000 | 0.000 | 0.000 |
| BRACE2015058 | 24.301 | 0.000 | 5.447 | 6.224 | 0.000 | 8.836 | 0.000 | 0.000 | 0.000 | 8.947 | 0.000 | 0.000 | 19.449 |
| BRACE2015314 | 0.000 | 0.000 | 0.000 | 100.000 | 0.000 | 0.000 | 0.000 | 0.000 | 0.000 | 0.000 | 0.000 | 0.000 | 0.000 |
| BRACE2016981 | 0.000 | 0.000 | 0.000 | 16.089 | 0.000 | 0.000 | 0.000 | 0.000 | 0.000 | 0.000 | 0.000 | 0.000 | 0.000 |
| BRACE2018762 | 0.000 | 0.000 | 0.000 | 41.027 | 0.000 | 0.000 | 0.000 | 0.000 | 0.000 | 58.973 | 0.000 | 0.000 | 0.000 |
| BRACE2024627 | 0.000 | 0.000 | 0.000 | 8.689 | 0.000 | 0.000 | 0.000 | 0.000 | 0.000 | 0.000 | 0.000 | 0.000 | 0.000 |
| BRACE2026836 | 0.000 | 0.000 | 0.000 | 4.898 | 0.000 | 0.000 | 0.000 | 0.000 | 0.000 | 0.000 | 0.000 | 0.000 | 0.000 |
| BRACE2027258 | 0.000 | 0.000 | 41.974 | 23.981 | 0.000 | 34.044 | 0.000 | 0.000 | 0.000 | 0.000 | 0.000 | 0.000 | 0.000 |
| BRACE2027970 | 0.000 | 0.000 | 0.000 | 45.981 | 0.000 | 0.000 | 0.000 | 0.000 | 0.000 | 0.000 | 0.000 | 0.000 | 0.000 |
| BRACE2028970 | 0.000 | 0.000 | 0.000 | 100.000 | 0.000 | 0.000 | 0.000 | 0.000 | 0.000 | 0.000 | 0.000 | 0.000 | 0.000 |
| BRACE2029112 | 0.000 | 0.000 | 0.000 | 100.000 | 0.000 | 0.000 | 0.000 | 0.000 | 0.000 | 0.000 | 0.000 | 0.000 | 0.000 |
| BRACE2029849 | 0.000 | 0.000 | 0.000 | 100.000 | 0.000 | 0.000 | 0.000 | 0.000 | 0.000 | 0.000 | 0.000 | 0.000 | 0.000 |
| BRACE2030326 | 0.000 | 0.000 | 0.000 | 100.000 | 0.000 | 0.000 | 0.000 | 0.000 | 0.000 | 0.000 | 0.000 | 0.000 | 0.000 |
| BRACE2030341 | 0.000 | 17.242 | 0.000 | 4.909 | 0.000 | 6.969 | 0.000 | 0.000 | 0.000 | 7.057 | 0.000 | 0.000 | 0.000 |
| BRACE2030884 | 0.000 | 0.000 | 0.000 | 100.000 | 0.000 | 0.000 | 0.000 | 0.000 | 0.000 | 0.000 | 0.000 | 0.000 | 0.000 |
| BRACE2031154 | 0.000 | 0.000 | 0.000 | 39.025 | 0.000 | 0.000 | 0.000 | 0.000 | 0.000 | 0.000 | 0.000 | 0.000 | 60.975 |
| BRACE2031389 | 0.000 | 25.357 | 0.000 | 7.220 | 0.000 | 30.747 | 0.000 | 0.000 | 0.000 | 0.000 | 0.000 | 36.677 | 0.000 |
| BRACE2031527 | 0.000 | 0.000 | 0.000 | 100.000 | 0.000 | 0.000 | 0.000 | 0.000 | 0.000 | 0.000 | 0.000 | 0.000 | 0.000 |
| BRACE2031531 | 0.000 | 0.000 | 0.000 | 100.000 | 0.000 | 0.000 | 0.000 | 0.000 | 0.000 | 0.000 | 0.000 | 0.000 | 0.000 |
| BRACE2031899 | 0.000 | 0.000 | 0.000 | 100.000 | 0.000 | 0.000 | 0.000 | 0.000 | 0.000 | 0.000 | 0.000 | 0.000 | 0.000 |
| BRACE2032044 | 0.000 | 0.000 | 0.000 | 100.000 | 0.000 | 0.000 | 0.000 | 0.000 | 0.000 | 0.000 | 0.000 | 0.000 | 0.000 |
| BRACE2032329 | 0.000 | 0.000 | 0.000 | 100.000 | 0.000 | 0.000 | 0.000 | 0.000 | 0.000 | 0.000 | 0.000 | 0.000 | 0.000 |
| BRACE2032385 | 0.000 | 0.000 | 0.000 | 100.000 | 0.000 | 0.000 | 0.000 | 0.000 | 0.000 | 0.000 | 0.000 | 0.000 | 0.000 |
| BRACE2032538 | 0.000 | 0.000 | 0.000 | 100.000 | 0.000 | 0.000 | 0.000 | 0.000 | 0.000 | 0.000 | 0.000 | 0.000 | 0.000 |
| BRACE2032823 | 0.000 | 0.000 | 0.000 | 100.000 | 0.000 | 0.000 | 0.000 | 0.000 | 0.000 | 0.000 | 0.000 | 0.000 | 0.000 |
| BRACE2033720 | 0.000 | 0.000 | 0.000 | 100.000 | 0.000 | 0.000 | 0.000 | 0.000 | 0.000 | 0.000 | 0.000 | 0.000 | 0.000 |
| BRACE2035381 | 0.000 | 0.000 | 0.000 | 36.117 | 0.000 | 10.254 | 10.177 | 0.000 | 0.000 | 20.766 | 0.000 | 0.000 | 11.286 |
| BRACE2035441 | 0.000 | 0.000 | 0.000 | 100.000 | 0.000 | 0.000 | 0.000 | 0.000 | 0.000 | 0.000 | 0.000 | 0.000 | 0.000 |
| BRACE2036005 | 0.000 | 0.000 | 0.000 | 100.000 | 0.000 | 0.000 | 0.000 | 0.000 | 0.000 | 0.000 | 0.000 | 0.000 | 0.000 |
| BRACE2036096 | 0.000 | 0.000 | 0.000 | 100.000 | 0.000 | 0.000 | 0.000 | 0.000 | 0.000 | 0.000 | 0.000 | 0.000 | 0.000 |
| BRACE2036830 | 0.000 | 0.000 | 0.000 | 100.000 | 0.000 | 0.000 | 0.000 | 0.000 | 0.000 | 0.000 | 0.000 | 0.000 | 0.000 |
| BRACE2036834 | 0.000 | 0.000 | 0.000 | 100.000 | 0.000 | 0.000 | 0.000 | 0.000 | 0.000 | 0.000 | 0.000 | 0.000 | 0.000 |
| BRACE2037847 | 0.000 | 0.000 | 0.000 | 100.000 | 0.000 | 0.000 | 0.000 | 0.000 | 0.000 | 0.000 | 0.000 | 0.000 | 0.000 |
| BRACE2038114 | 0.000 | 0.000 | 0.000 | 100.000 | 0.000 | 0.000 | 0.000 | 0.000 | 0.000 | 0.000 | 0.000 | 0.000 | 0.000 |
| BRACE2038329 | 0.000 | 0.000 | 0.000 | 100.000 | 0.000 | 0.000 | 0.000 | 0.000 | 0.000 | 0.000 | 0.000 | 0.000 | 0.000 |
| BRACE2038551 | 0.000 | 0.000 | 0.000 | 100.000 | 0.000 | 0.000 | 0.000 | 0.000 | 0.000 | 0.000 | 0.000 | 0.000 | 0.000 |
| BRACE2039249 | 0.000 | 0.000 | 0.000 | 100.000 | 0.000 | 0.000 | 0.000 | 0.000 | 0.000 | 0.000 | 0.000 | 0.000 | 0.000 |
| BRACE2039327 | 0.000 | 0.000 | 0.000 | 100.000 | 0.000 | 0.000 | 0.000 | 0.000 | 0.000 | 0.000 | 0.000 | 0.000 | 0.000 |
| BRACE2039475 | 0.000 | 0.000 | 0.000 | 100.000 | 0.000 | 0.000 | 0.000 | 0.000 | 0.000 | 0.000 | 0.000 | 0.000 | 0.000 |
| BRACE2039734 | 0.000 | 0.000 | 0.000 | 100.000 | 0.000 | 0.000 | 0.000 | 0.000 | 0.000 | 0.000 | 0.000 | 0.000 | 0.000 |
| BRACE2040138 | 0.000 | 0.000 | 0.000 | 100.000 | 0.000 | 0.000 | 0.000 | 0.000 | 0.000 | 0.000 | 0.000 | 0.000 | 0.000 |
| BRACE2040325 | 0.000 | 0.000 | 0.000 | 100.000 | 0.000 | 0.000 | 0.000 | 0.000 | 0.000 | 0.000 | 0.000 | 0.000 | 0.000 |
| BRACE2041009 | 0.000 | 0.000 | 0.000 | 100.000 | 0.000 | 0.000 | 0.000 | 0.000 | 0.000 | 0.000 | 0.000 | 0.000 | 0.000 |
| BRACE2041200 | 0.000 | 0.000 | 0.000 | 38.787 | 0.000 | 0.000 | 0.000 | 0.000 | 0.000 | 0.000 | 0.000 | 0.000 | 0.000 |
| BRACE2041264 | 0.000 | 0.000 | 0.000 | 100.000 | 0.000 | 0.000 | 0.000 | 0.000 | 0.000 | 0.000 | 0.000 | 0.000 | 0.000 |
| BRACE2042550 | 0.000 | 0.000 | 0.000 | 100.000 | 0.000 | 0.000 | 0.000 | 0.000 | 0.000 | 0.000 | 0.000 | 0.000 | 0.000 |
| BRACE2043142 | 0.000 | 0.000 | 0.000 | 52.057 | 0.000 | 0.000 | 0.000 | 0.000 | 0.000 | 0.000 | 0.000 | 0.000 | 0.000 |
| BRACE2043248 | 0.000 | 0.000 | 0.000 | 100.000 | 0.000 | 0.000 | 0.000 | 0.000 | 0.000 | 0.000 | 0.000 | 0.000 | 0.000 |
| BRACE2043349 | 0.000 | 0.000 | 0.000 | 100.000 | 0.000 | 0.000 | 0.000 | 0.000 | 0.000 | 0.000 | 0.000 | 0.000 | 0.000 |
| BRACE2043665 | 0.000 | 0.000 | 0.000 | 16.915 | 0.000 | 0.000 | 0.000 | 0.000 | 0.000 | 0.000 | 0.000 | 0.000 | 0.000 |
| BRACE2044286 | 0.000 | 0.000 | 0.000 | 26.499 | 0.000 | 0.000 | 18.667 | 0.000 | 0.000 | 0.000 | 0.000 | 0.000 | 0.000 |
| BRACE2044816 | 0.000 | 0.000 | 0.000 | 100.000 | 0.000 | 0.000 | 0.000 | 0.000 | 0.000 | 0.000 | 0.000 | 0.000 | 0.000 |
| BRACE2044949 | 0.000 | 0.000 | 0.000 | 2.766 | 0.000 | 0.000 | 0.000 | 0.000 | 0.000 | 0.000 | 0.000 | 0.000 | 0.000 |
| BRACE2045300 | 0.000 | 0.000 | 0.000 | 100.000 | 0.000 | 0.000 | 0.000 | 0.000 | 0.000 | 0.000 | 0.000 | 0.000 | 0.000 |
| BRACE2045428 | 0.000 | 0.000 | 0.000 | 100.000 | 0.000 | 0.000 | 0.000 | 0.000 | 0.000 | 0.000 | 0.000 | 0.000 | 0.000 |
| BRACE2045596 | 0.000 | 0.000 | 0.000 | 100.000 | 0.000 | 0.000 | 0.000 | 0.000 | 0.000 | 0.000 | 0.000 | 0.000 | 0.000 |
| BRACE2045772 | 0.000 | 0.000 | 0.000 | 2.055 | 0.000 | 0.000 | 0.000 | 0.000 | 0.000 | 0.000 | 0.000 | 0.000 | 0.000 |
| BRACE2045947 | 0.000 | 0.000 | 0.000 | 100.000 | 0.000 | 0.000 | 0.000 | 0.000 | 0.000 | 0.000 | 0.000 | 0.000 | 0.000 |
| BRACE2045954 | 0.000 | 0.000 | 33.925 | 38.766 | 0.000 | 0.000 | 27.309 | 0.000 | 0.000 | 0.000 | 0.000 | 0.000 | 0.000 |
| BRACE2046251 | 0.000 | 0.000 | 0.000 | 100.000 | 0.000 | 0.000 | 0.000 | 0.000 | 0.000 | 0.000 | 0.000 | 0.000 | 0.000 |
| BRACE2046295 | 0.000 | 0.000 | 0.000 | 100.000 | 0.000 | 0.000 | 0.000 | 0.000 | 0.000 | 0.000 | 0.000 | 0.000 | 0.000 |
| BRACE2047011 | 0.000 | 0.000 | 0.000 | 100.000 | 0.000 | 0.000 | 0.000 | 0.000 | 0.000 | 0.000 | 0.000 | 0.000 | 0.000 |
| BRACE2047350 | 0.000 | 0.000 | 0.000 | 45.981 | 0.000 | 0.000 | 0.000 | 0.000 | 0.000 | 0.000 | 0.000 | 0.000 | 0.000 |
| BRACE2047377 | 0.000 | 0.000 | 0.000 | 100.000 | 0.000 | 0.000 | 0.000 | 0.000 | 0.000 | 0.000 | 0.000 | 0.000 | 0.000 |
| BRACE2047385 | 0.000 | 0.000 | 0.000 | 100.000 | 0.000 | 0.000 | 0.000 | 0.000 | 0.000 | 0.000 | 0.000 | 0.000 | 0.000 |
| BRACE3000071 | 0.000 | 0.000 | 0.000 | 9.532 | 0.000 | 0.000 | 0.000 | 0.000 | 0.000 | 0.000 | 0.000 | 0.000 | 0.000 |
| BRACE3000697 | 0.000 | 0.000 | 0.000 | 100.000 | 0.000 | 0.000 | 0.000 | 0.000 | 0.000 | 0.000 | 0.000 | 0.000 | 0.000 |
| BRACE3000787 | 0.000 | 0.000 | 0.000 | 15.541 | 0.000 | 0.000 | 32.844 | 0.000 | 0.000 | 0.000 | 0.000 | 39.475 | 12.141 |
| BRACE3000840 | 0.000 | 0.000 | 0.000 | 100.000 | 0.000 | 0.000 | 0.000 | 0.000 | 0.000 | 0.000 | 0.000 | 0.000 | 0.000 |
| BRACE3000973 | 0.000 | 0.000 | 0.000 | 28.699 | 0.000 | 13.581 | 0.000 | 0.000 | 0.000 | 0.000 | 0.000 | 0.000 | 0.000 |
| BRACE3001002 | 0.000 | 0.000 | 0.000 | 100.000 | 0.000 | 0.000 | 0.000 | 0.000 | 0.000 | 0.000 | 0.000 | 0.000 | 0.000 |
| BRACE3001217 | 0.000 | 0.000 | 0.000 | 100.000 | 0.000 | 0.000 | 0.000 | 0.000 | 0.000 | 0.000 | 0.000 | 0.000 | 0.000 |
| BRACE3001391 | 0.000 | 0.000 | 0.000 | 100.000 | 0.000 | 0.000 | 0.000 | 0.000 | 0.000 | 0.000 | 0.000 | 0.000 | 0.000 |
| BRACE3001595 | 0.000 | 0.000 | 0.000 | 100.000 | 0.000 | 0.000 | 0.000 | 0.000 | 0.000 | 0.000 | 0.000 | 0.000 | 0.000 |
| BRACE3001754 | 0.000 | 0.000 | 0.000 | 38.787 | 0.000 | 0.000 | 0.000 | 0.000 | 0.000 | 0.000 | 0.000 | 0.000 | 0.000 |
| BRACE3002298 | 0.000 | 0.000 | 0.000 | 100.000 | 0.000 | 0.000 | 0.000 | 0.000 | 0.000 | 0.000 | 0.000 | 0.000 | 0.000 |
| BRACE3002390 | 0.000 | 0.000 | 0.000 | 100.000 | 0.000 | 0.000 | 0.000 | 0.000 | 0.000 | 0.000 | 0.000 | 0.000 | 0.000 |
| BRACE3002508 | 0.000 | 0.000 | 0.000 | 100.000 | 0.000 | 0.000 | 0.000 | 0.000 | 0.000 | 0.000 | 0.000 | 0.000 | 0.000 |
| BRACE3003004 | 0.000 | 0.000 | 0.000 | 100.000 | 0.000 | 0.000 | 0.000 | 0.000 | 0.000 | 0.000 | 0.000 | 0.000 | 0.000 |
| BRACE3003192 | 0.000 | 0.000 | 0.000 | 41.512 | 0.000 | 0.000 | 58.488 | 0.000 | 0.000 | 0.000 | 0.000 | 0.000 | 0.000 |
| BRACE3003595 | 0.000 | 0.000 | 0.000 | 100.000 | 0.000 | 0.000 | 0.000 | 0.000 | 0.000 | 0.000 | 0.000 | 0.000 | 0.000 |
| BRACE3003698 | 0.000 | 0.000 | 0.000 | 45.981 | 0.000 | 0.000 | 0.000 | 0.000 | 0.000 | 0.000 | 0.000 | 0.000 | 0.000 |
| BRACE3004058 | 0.000 | 0.000 | 0.000 | 100.000 | 0.000 | 0.000 | 0.000 | 0.000 | 0.000 | 0.000 | 0.000 | 0.000 | 0.000 |
| BRACE3004113 | 0.000 | 0.000 | 0.000 | 100.000 | 0.000 | 0.000 | 0.000 | 0.000 | 0.000 | 0.000 | 0.000 | 0.000 | 0.000 |
| BRACE3004150 | 7.175 | 0.000 | 0.000 | 2.757 | 0.000 | 0.000 | 0.000 | 0.000 | 0.000 | 0.000 | 0.000 | 0.000 | 0.000 |
| BRACE3004358 | 0.000 | 0.000 | 0.000 | 100.000 | 0.000 | 0.000 | 0.000 | 0.000 | 0.000 | 0.000 | 0.000 | 0.000 | 0.000 |
| BRACE3004435 | 0.000 | 0.000 | 0.000 | 100.000 | 0.000 | 0.000 | 0.000 | 0.000 | 0.000 | 0.000 | 0.000 | 0.000 | 0.000 |
| BRACE3004772 | 0.000 | 0.000 | 0.000 | 100.000 | 0.000 | 0.000 | 0.000 | 0.000 | 0.000 | 0.000 | 0.000 | 0.000 | 0.000 |
| BRACE3004783 | 0.000 | 0.000 | 0.000 | 100.000 | 0.000 | 0.000 | 0.000 | 0.000 | 0.000 | 0.000 | 0.000 | 0.000 | 0.000 |
| BRACE3004843 | 0.000 | 0.000 | 0.000 | 22.581 | 0.000 | 0.000 | 0.000 | 0.000 | 0.000 | 0.000 | 0.000 | 0.000 | 0.000 |
| BRACE3004880 | 0.000 | 0.000 | 0.000 | 100.000 | 0.000 | 0.000 | 0.000 | 0.000 | 0.000 | 0.000 | 0.000 | 0.000 | 0.000 |
| BRACE3005145 | 0.000 | 0.000 | 0.000 | 100.000 | 0.000 | 0.000 | 0.000 | 0.000 | 0.000 | 0.000 | 0.000 | 0.000 | 0.000 |
| BRACE3005225 | 0.000 | 0.000 | 0.000 | 100.000 | 0.000 | 0.000 | 0.000 | 0.000 | 0.000 | 0.000 | 0.000 | 0.000 | 0.000 |
| BRACE3005430 | 0.000 | 0.000 | 0.000 | 100.000 | 0.000 | 0.000 | 0.000 | 0.000 | 0.000 | 0.000 | 0.000 | 0.000 | 0.000 |
| BRACE3005499 | 0.000 | 0.000 | 0.000 | 22.199 | 0.000 | 0.000 | 31.276 | 0.000 | 0.000 | 0.000 | 0.000 | 0.000 | 0.000 |
| BRACE3006185 | 0.000 | 0.000 | 0.000 | 100.000 | 0.000 | 0.000 | 0.000 | 0.000 | 0.000 | 0.000 | 0.000 | 0.000 | 0.000 |
| BRACE3006226 | 0.000 | 0.000 | 0.000 | 100.000 | 0.000 | 0.000 | 0.000 | 0.000 | 0.000 | 0.000 | 0.000 | 0.000 | 0.000 |
| BRACE3006462 | 0.000 | 0.000 | 0.000 | 100.000 | 0.000 | 0.000 | 0.000 | 0.000 | 0.000 | 0.000 | 0.000 | 0.000 | 0.000 |
| BRACE3006872 | 0.000 | 0.000 | 0.000 | 100.000 | 0.000 | 0.000 | 0.000 | 0.000 | 0.000 | 0.000 | 0.000 | 0.000 | 0.000 |
| BRACE3007322 | 0.000 | 0.000 | 0.000 | 100.000 | 0.000 | 0.000 | 0.000 | 0.000 | 0.000 | 0.000 | 0.000 | 0.000 | 0.000 |
| BRACE3007472 | 0.000 | 0.000 | 0.000 | 100.000 | 0.000 | 0.000 | 0.000 | 0.000 | 0.000 | 0.000 | 0.000 | 0.000 | 0.000 |
| BRACE3007480 | 0.000 | 0.000 | 0.000 | 13.820 | 0.000 | 6.540 | 19.471 | 0.000 | 0.000 | 52.973 | 0.000 | 0.000 | 7.197 |
| BRACE3007559 | 0.000 | 0.000 | 0.000 | 100.000 | 0.000 | 0.000 | 0.000 | 0.000 | 0.000 | 0.000 | 0.000 | 0.000 | 0.000 |
| BRACE3007625 | 0.000 | 0.000 | 0.000 | 100.000 | 0.000 | 0.000 | 0.000 | 0.000 | 0.000 | 0.000 | 0.000 | 0.000 | 0.000 |
| BRACE3007642 | 0.000 | 0.000 | 0.000 | 100.000 | 0.000 | 0.000 | 0.000 | 0.000 | 0.000 | 0.000 | 0.000 | 0.000 | 0.000 |
| BRACE3007767 | 0.000 | 0.000 | 0.000 | 100.000 | 0.000 | 0.000 | 0.000 | 0.000 | 0.000 | 0.000 | 0.000 | 0.000 | 0.000 |
| BRACE3008036 | 0.000 | 0.000 | 0.000 | 13.098 | 0.000 | 18.594 | 0.000 | 0.000 | 0.000 | 0.000 | 0.000 | 0.000 | 0.000 |
| BRACE3008092 | 0.000 | 0.000 | 0.000 | 100.000 | 0.000 | 0.000 | 0.000 | 0.000 | 0.000 | 0.000 | 0.000 | 0.000 | 0.000 |
| BRACE3008137 | 0.000 | 0.000 | 0.000 | 100.000 | 0.000 | 0.000 | 0.000 | 0.000 | 0.000 | 0.000 | 0.000 | 0.000 | 0.000 |
| BRACE3008384 | 0.000 | 0.000 | 0.000 | 56.141 | 0.000 | 0.000 | 0.000 | 0.000 | 0.000 | 0.000 | 0.000 | 0.000 | 43.859 |
| BRACE3008720 | 0.000 | 0.000 | 0.000 | 100.000 | 0.000 | 0.000 | 0.000 | 0.000 | 0.000 | 0.000 | 0.000 | 0.000 | 0.000 |
| BRACE3008772 | 0.000 | 0.000 | 0.000 | 37.308 | 0.000 | 0.000 | 0.000 | 0.000 | 0.000 | 0.000 | 0.000 | 0.000 | 0.000 |
| BRACE3009090 | 0.000 | 0.000 | 0.000 | 100.000 | 0.000 | 0.000 | 0.000 | 0.000 | 0.000 | 0.000 | 0.000 | 0.000 | 0.000 |
| BRACE3009237 | 0.000 | 0.000 | 0.000 | 12.875 | 0.000 | 0.000 | 18.139 | 0.000 | 0.000 | 37.013 | 0.000 | 0.000 | 20.116 |
| BRACE3009297 | 0.000 | 0.000 | 0.000 | 100.000 | 0.000 | 0.000 | 0.000 | 0.000 | 0.000 | 0.000 | 0.000 | 0.000 | 0.000 |
| BRACE3009377 | 0.000 | 0.000 | 0.000 | 100.000 | 0.000 | 0.000 | 0.000 | 0.000 | 0.000 | 0.000 | 0.000 | 0.000 | 0.000 |
| BRACE3009574 | 0.000 | 0.000 | 0.000 | 100.000 | 0.000 | 0.000 | 0.000 | 0.000 | 0.000 | 0.000 | 0.000 | 0.000 | 0.000 |
| BRACE3009701 | 0.000 | 0.000 | 0.000 | 100.000 | 0.000 | 0.000 | 0.000 | 0.000 | 0.000 | 0.000 | 0.000 | 0.000 | 0.000 |
| BRACE3009708 | 0.000 | 0.000 | 0.000 | 100.000 | 0.000 | 0.000 | 0.000 | 0.000 | 0.000 | 0.000 | 0.000 | 0.000 | 0.000 |
| BRACE3009724 | 0.000 | 0.000 | 0.000 | 41.512 | 0.000 | 0.000 | 58.488 | 0.000 | 0.000 | 0.000 | 0.000 | 0.000 | 0.000 |
| BRACE3009747 | 4.133 | 0.000 | 2.779 | 1.588 | 0.000 | 0.000 | 2.237 | 0.000 | 0.000 | 2.283 | 8.327 | 0.000 | 2.481 |
| BRACE3010397 | 0.000 | 0.000 | 0.000 | 24.243 | 0.000 | 0.000 | 0.000 | 0.000 | 0.000 | 0.000 | 0.000 | 0.000 | 75.757 |
| BRACE3010428 | 6.344 | 2.854 | 4.266 | 3.250 | 0.000 | 3.460 | 6.868 | 0.000 | 0.000 | 2.336 | 4.261 | 0.000 | 2.539 |
| BRACE3011271 | 0.000 | 0.000 | 27.420 | 39.165 | 0.000 | 11.120 | 11.036 | 0.000 | 0.000 | 11.259 | 0.000 | 0.000 | 0.000 |
| BRACE3011421 | 0.000 | 0.000 | 0.000 | 40.103 | 0.000 | 0.000 | 28.251 | 0.000 | 0.000 | 0.000 | 0.000 | 0.000 | 0.000 |
| BRACE3011505 | 0.000 | 0.000 | 0.000 | 100.000 | 0.000 | 0.000 | 0.000 | 0.000 | 0.000 | 0.000 | 0.000 | 0.000 | 0.000 |
| BRACE3012364 | 0.000 | 10.603 | 0.000 | 6.038 | 0.000 | 0.000 | 8.506 | 0.000 | 15.067 | 4.339 | 0.000 | 0.000 | 9.433 |
| BRACE3012930 | 0.000 | 0.000 | 0.000 | 38.787 | 0.000 | 0.000 | 0.000 | 0.000 | 0.000 | 0.000 | 0.000 | 0.000 | 0.000 |
| BRACE3013119 | 0.000 | 0.000 | 0.000 | 100.000 | 0.000 | 0.000 | 0.000 | 0.000 | 0.000 | 0.000 | 0.000 | 0.000 | 0.000 |
| BRACE3013576 | 0.000 | 0.000 | 0.000 | 100.000 | 0.000 | 0.000 | 0.000 | 0.000 | 0.000 | 0.000 | 0.000 | 0.000 | 0.000 |
| BRACE3013740 | 0.000 | 0.000 | 0.000 | 100.000 | 0.000 | 0.000 | 0.000 | 0.000 | 0.000 | 0.000 | 0.000 | 0.000 | 0.000 |
| BRACE3013780 | 0.000 | 11.957 | 5.958 | 3.404 | 0.000 | 4.833 | 0.000 | 0.000 | 0.000 | 0.000 | 17.852 | 0.000 | 0.000 |
| BRACE3014005 | 0.000 | 0.000 | 0.000 | 100.000 | 0.000 | 0.000 | 0.000 | 0.000 | 0.000 | 0.000 | 0.000 | 0.000 | 0.000 |
| BRACE3014068 | 0.000 | 0.000 | 0.000 | 100.000 | 0.000 | 0.000 | 0.000 | 0.000 | 0.000 | 0.000 | 0.000 | 0.000 | 0.000 |
| BRACE3014231 | 0.000 | 0.000 | 0.000 | 100.000 | 0.000 | 0.000 | 0.000 | 0.000 | 0.000 | 0.000 | 0.000 | 0.000 | 0.000 |
| BRACE3014317 | 0.000 | 0.000 | 0.000 | 100.000 | 0.000 | 0.000 | 0.000 | 0.000 | 0.000 | 0.000 | 0.000 | 0.000 | 0.000 |
| BRACE3014807 | 0.000 | 0.000 | 0.000 | 100.000 | 0.000 | 0.000 | 0.000 | 0.000 | 0.000 | 0.000 | 0.000 | 0.000 | 0.000 |
| BRACE3015027 | 4.794 | 0.000 | 6.448 | 9.209 | 0.000 | 0.000 | 0.000 | 0.000 | 0.000 | 0.000 | 0.000 | 0.000 | 0.000 |
| BRACE3015121 | 0.000 | 0.000 | 0.000 | 100.000 | 0.000 | 0.000 | 0.000 | 0.000 | 0.000 | 0.000 | 0.000 | 0.000 | 0.000 |
| BRACE3015262 | 0.000 | 0.000 | 0.000 | 100.000 | 0.000 | 0.000 | 0.000 | 0.000 | 0.000 | 0.000 | 0.000 | 0.000 | 0.000 |
| BRACE3015521 | 0.000 | 0.000 | 0.000 | 100.000 | 0.000 | 0.000 | 0.000 | 0.000 | 0.000 | 0.000 | 0.000 | 0.000 | 0.000 |
| BRACE3015894 | 0.000 | 0.000 | 0.000 | 100.000 | 0.000 | 0.000 | 0.000 | 0.000 | 0.000 | 0.000 | 0.000 | 0.000 | 0.000 |
| BRACE3016884 | 0.000 | 0.000 | 0.000 | 9.165 | 0.000 | 0.000 | 0.000 | 0.000 | 0.000 | 0.000 | 0.000 | 0.000 | 0.000 |
| BRACE3018308 | 0.000 | 0.000 | 0.000 | 100.000 | 0.000 | 0.000 | 0.000 | 0.000 | 0.000 | 0.000 | 0.000 | 0.000 | 0.000 |
| BRACE3018963 | 0.000 | 0.000 | 0.000 | 41.027 | 0.000 | 0.000 | 0.000 | 0.000 | 0.000 | 58.973 | 0.000 | 0.000 | 0.000 |
| BRACE3019055 | 0.000 | 0.000 | 0.000 | 100.000 | 0.000 | 0.000 | 0.000 | 0.000 | 0.000 | 0.000 | 0.000 | 0.000 | 0.000 |
| BRACE3019084 | 0.000 | 0.000 | 0.000 | 100.000 | 0.000 | 0.000 | 0.000 | 0.000 | 0.000 | 0.000 | 0.000 | 0.000 | 0.000 |
| BRACE3020194 | 0.000 | 0.000 | 0.000 | 100.000 | 0.000 | 0.000 | 0.000 | 0.000 | 0.000 | 0.000 | 0.000 | 0.000 | 0.000 |
| BRACE3020286 | 0.000 | 0.000 | 0.000 | 100.000 | 0.000 | 0.000 | 0.000 | 0.000 | 0.000 | 0.000 | 0.000 | 0.000 | 0.000 |
| BRACE3020594 | 0.000 | 0.000 | 0.000 | 100.000 | 0.000 | 0.000 | 0.000 | 0.000 | 0.000 | 0.000 | 0.000 | 0.000 | 0.000 |
| BRACE3022769 | 2.639 | 24.924 | 10.646 | 9.124 | 0.000 | 4.318 | 4.285 | 3.277 | 0.000 | 4.372 | 5.316 | 0.000 | 4.752 |
| BRACE3023912 | 0.000 | 0.000 | 0.000 | 100.000 | 0.000 | 0.000 | 0.000 | 0.000 | 0.000 | 0.000 | 0.000 | 0.000 | 0.000 |
| BRACE3024073 | 0.000 | 0.000 | 0.000 | 100.000 | 0.000 | 0.000 | 0.000 | 0.000 | 0.000 | 0.000 | 0.000 | 0.000 | 0.000 |
| BRACE3024659 | 0.000 | 0.000 | 0.000 | 100.000 | 0.000 | 0.000 | 0.000 | 0.000 | 0.000 | 0.000 | 0.000 | 0.000 | 0.000 |
| BRACE3024662 | 0.000 | 0.000 | 0.000 | 100.000 | 0.000 | 0.000 | 0.000 | 0.000 | 0.000 | 0.000 | 0.000 | 0.000 | 0.000 |
| BRACE3025153 | 0.000 | 0.000 | 0.000 | 100.000 | 0.000 | 0.000 | 0.000 | 0.000 | 0.000 | 0.000 | 0.000 | 0.000 | 0.000 |
| BRACE3025457 | 0.000 | 0.000 | 0.000 | 100.000 | 0.000 | 0.000 | 0.000 | 0.000 | 0.000 | 0.000 | 0.000 | 0.000 | 0.000 |
| BRACE3025531 | 0.000 | 0.000 | 0.000 | 100.000 | 0.000 | 0.000 | 0.000 | 0.000 | 0.000 | 0.000 | 0.000 | 0.000 | 0.000 |
| BRACE3025630 | 0.000 | 0.000 | 0.000 | 100.000 | 0.000 | 0.000 | 0.000 | 0.000 | 0.000 | 0.000 | 0.000 | 0.000 | 0.000 |
| BRACE3026008 | 0.000 | 0.000 | 0.000 | 100.000 | 0.000 | 0.000 | 0.000 | 0.000 | 0.000 | 0.000 | 0.000 | 0.000 | 0.000 |
| BRACE3026075 | 0.000 | 0.000 | 0.000 | 100.000 | 0.000 | 0.000 | 0.000 | 0.000 | 0.000 | 0.000 | 0.000 | 0.000 | 0.000 |
| BRACE3026735 | 0.000 | 0.000 | 0.000 | 51.471 | 0.000 | 24.356 | 24.173 | 0.000 | 0.000 | 0.000 | 0.000 | 0.000 | 0.000 |
| BRACE3027242 | 0.000 | 0.000 | 0.000 | 100.000 | 0.000 | 0.000 | 0.000 | 0.000 | 0.000 | 0.000 | 0.000 | 0.000 | 0.000 |
| BRACE3027326 | 0.000 | 0.000 | 0.000 | 100.000 | 0.000 | 0.000 | 0.000 | 0.000 | 0.000 | 0.000 | 0.000 | 0.000 | 0.000 |
| BRACE3027478 | 0.000 | 0.000 | 0.000 | 11.360 | 0.000 | 16.126 | 0.000 | 0.000 | 0.000 | 0.000 | 0.000 | 0.000 | 35.497 |
| BRACE3030103 | 0.000 | 0.000 | 0.000 | 100.000 | 0.000 | 0.000 | 0.000 | 0.000 | 0.000 | 0.000 | 0.000 | 0.000 | 0.000 |
| BRACE3031838 | 0.000 | 0.000 | 0.000 | 41.512 | 0.000 | 0.000 | 58.488 | 0.000 | 0.000 | 0.000 | 0.000 | 0.000 | 0.000 |
| BRACE3032983 | 0.000 | 0.000 | 0.000 | 100.000 | 0.000 | 0.000 | 0.000 | 0.000 | 0.000 | 0.000 | 0.000 | 0.000 | 0.000 |
| BRACE3040856 | 0.000 | 0.000 | 0.000 | 100.000 | 0.000 | 0.000 | 0.000 | 0.000 | 0.000 | 0.000 | 0.000 | 0.000 | 0.000 |
| BRACE3045033 | 0.000 | 0.000 | 0.000 | 100.000 | 0.000 | 0.000 | 0.000 | 0.000 | 0.000 | 0.000 | 0.000 | 0.000 | 0.000 |
| BRALZ2011796 | 0.000 | 0.000 | 0.000 | 3.912 | 39.830 | 0.000 | 5.511 | 0.000 | 19.525 | 0.000 | 20.514 | 0.000 | 6.112 |
| BRALZ2012183 | 0.000 | 0.000 | 0.000 | 0.000 | 100.000 | 0.000 | 0.000 | 0.000 | 0.000 | 0.000 | 0.000 | 0.000 | 0.000 |
| BRALZ2012848 | 0.000 | 0.000 | 0.000 | 0.000 | 100.000 | 0.000 | 0.000 | 0.000 | 0.000 | 0.000 | 0.000 | 0.000 | 0.000 |
| BRALZ2014484 | 0.000 | 0.000 | 0.000 | 0.000 | 100.000 | 0.000 | 0.000 | 0.000 | 0.000 | 0.000 | 0.000 | 0.000 | 0.000 |
| BRALZ2016085 | 0.000 | 0.000 | 0.000 | 0.000 | 100.000 | 0.000 | 0.000 | 0.000 | 0.000 | 0.000 | 0.000 | 0.000 | 0.000 |
| BRALZ2016498 | 0.000 | 0.000 | 0.000 | 0.000 | 100.000 | 0.000 | 0.000 | 0.000 | 0.000 | 0.000 | 0.000 | 0.000 | 0.000 |
| BRALZ2017359 | 0.000 | 0.000 | 0.000 | 0.000 | 75.184 | 0.000 | 0.000 | 0.000 | 0.000 | 0.000 | 0.000 | 0.000 | 0.000 |
| BRAMY2001473 | 0.000 | 0.000 | 0.000 | 0.000 | 0.000 | 100.000 | 0.000 | 0.000 | 0.000 | 0.000 | 0.000 | 0.000 | 0.000 |
| BRAMY2003008 | 0.000 | 0.000 | 0.000 | 0.000 | 0.000 | 26.646 | 26.445 | 0.000 | 0.000 | 0.000 | 0.000 | 0.000 | 0.000 |
| BRAMY2004771 | 0.000 | 0.000 | 0.000 | 0.000 | 0.000 | 100.000 | 0.000 | 0.000 | 0.000 | 0.000 | 0.000 | 0.000 | 0.000 |
| BRAMY2005052 | 0.000 | 0.000 | 0.000 | 8.242 | 0.000 | 46.801 | 11.612 | 0.000 | 0.000 | 0.000 | 0.000 | 0.000 | 25.755 |
| BRAMY2017528 | 0.000 | 0.000 | 0.000 | 0.000 | 0.000 | 100.000 | 0.000 | 0.000 | 0.000 | 0.000 | 0.000 | 0.000 | 0.000 |
| BRAMY2019300 | 0.000 | 0.000 | 0.000 | 0.000 | 0.000 | 50.189 | 49.811 | 0.000 | 0.000 | 0.000 | 0.000 | 0.000 | 0.000 |
| BRAMY2019963 | 0.000 | 0.000 | 25.378 | 0.000 | 0.000 | 6.861 | 20.428 | 0.000 | 0.000 | 6.947 | 0.000 | 0.000 | 7.551 |
| BRAMY2019985 | 0.000 | 0.000 | 0.000 | 0.000 | 0.000 | 100.000 | 0.000 | 0.000 | 0.000 | 0.000 | 0.000 | 0.000 | 0.000 |
| BRAMY2020058 | 0.000 | 0.000 | 0.000 | 19.826 | 0.000 | 28.146 | 0.000 | 0.000 | 0.000 | 0.000 | 0.000 | 0.000 | 0.000 |
| BRAMY2020270 | 0.000 | 0.000 | 0.000 | 0.000 | 0.000 | 100.000 | 0.000 | 0.000 | 0.000 | 0.000 | 0.000 | 0.000 | 0.000 |
| BRAMY2021498 | 0.000 | 0.000 | 0.000 | 0.000 | 0.000 | 100.000 | 0.000 | 0.000 | 0.000 | 0.000 | 0.000 | 0.000 | 0.000 |
| BRAMY2028856 | 0.000 | 0.000 | 0.000 | 0.000 | 0.000 | 100.000 | 0.000 | 0.000 | 0.000 | 0.000 | 0.000 | 0.000 | 0.000 |
| BRAMY2028914 | 0.000 | 0.000 | 0.000 | 0.000 | 0.000 | 17.849 | 0.000 | 0.000 | 0.000 | 0.000 | 0.000 | 0.000 | 19.645 |
| BRAMY2029602 | 0.000 | 0.000 | 0.000 | 0.000 | 0.000 | 100.000 | 0.000 | 0.000 | 0.000 | 0.000 | 0.000 | 0.000 | 0.000 |
| BRAMY2030098 | 0.000 | 0.000 | 0.000 | 29.935 | 0.000 | 42.496 | 0.000 | 0.000 | 0.000 | 0.000 | 0.000 | 0.000 | 0.000 |
| BRAMY2030109 | 0.000 | 0.000 | 0.000 | 0.000 | 0.000 | 100.000 | 0.000 | 0.000 | 0.000 | 0.000 | 0.000 | 0.000 | 0.000 |
| BRAMY2030702 | 0.000 | 0.000 | 0.000 | 0.000 | 0.000 | 100.000 | 0.000 | 0.000 | 0.000 | 0.000 | 0.000 | 0.000 | 0.000 |
| BRAMY2030703 | 0.000 | 0.000 | 0.000 | 0.000 | 0.000 | 100.000 | 0.000 | 0.000 | 0.000 | 0.000 | 0.000 | 0.000 | 0.000 |
| BRAMY2030799 | 0.000 | 0.000 | 0.000 | 0.000 | 0.000 | 100.000 | 0.000 | 0.000 | 0.000 | 0.000 | 0.000 | 0.000 | 0.000 |
| BRAMY2031317 | 0.000 | 0.000 | 0.000 | 33.400 | 0.000 | 15.805 | 0.000 | 0.000 | 0.000 | 16.004 | 0.000 | 0.000 | 34.791 |
| BRAMY2031377 | 0.000 | 0.000 | 0.000 | 0.000 | 0.000 | 100.000 | 0.000 | 0.000 | 0.000 | 0.000 | 0.000 | 0.000 | 0.000 |
| BRAMY2031442 | 0.000 | 0.000 | 0.000 | 0.000 | 0.000 | 100.000 | 0.000 | 0.000 | 0.000 | 0.000 | 0.000 | 0.000 | 0.000 |
| BRAMY2032014 | 0.000 | 0.000 | 0.000 | 0.000 | 0.000 | 100.000 | 0.000 | 0.000 | 0.000 | 0.000 | 0.000 | 0.000 | 0.000 |
| BRAMY2032242 | 0.000 | 0.000 | 0.000 | 0.000 | 0.000 | 100.000 | 0.000 | 0.000 | 0.000 | 0.000 | 0.000 | 0.000 | 0.000 |
| BRAMY2032317 | 0.000 | 0.000 | 0.000 | 0.000 | 0.000 | 60.652 | 0.000 | 0.000 | 0.000 | 0.000 | 0.000 | 0.000 | 0.000 |
| BRAMY2033003 | 0.000 | 0.000 | 0.000 | 0.000 | 0.000 | 100.000 | 0.000 | 0.000 | 0.000 | 0.000 | 0.000 | 0.000 | 0.000 |
| BRAMY2033116 | 0.000 | 0.000 | 0.000 | 0.000 | 0.000 | 100.000 | 0.000 | 0.000 | 0.000 | 0.000 | 0.000 | 0.000 | 0.000 |
| BRAMY2033267 | 0.000 | 0.000 | 0.000 | 0.000 | 0.000 | 100.000 | 0.000 | 0.000 | 0.000 | 0.000 | 0.000 | 0.000 | 0.000 |
| BRAMY2033594 | 0.000 | 0.000 | 0.000 | 0.000 | 0.000 | 100.000 | 0.000 | 0.000 | 0.000 | 0.000 | 0.000 | 0.000 | 0.000 |
| BRAMY2034185 | 0.000 | 0.000 | 0.000 | 0.000 | 0.000 | 100.000 | 0.000 | 0.000 | 0.000 | 0.000 | 0.000 | 0.000 | 0.000 |
| BRAMY2034920 | 0.000 | 0.000 | 0.000 | 0.000 | 0.000 | 100.000 | 0.000 | 0.000 | 0.000 | 0.000 | 0.000 | 0.000 | 0.000 |
| BRAMY2034993 | 0.000 | 0.000 | 0.000 | 0.000 | 0.000 | 100.000 | 0.000 | 0.000 | 0.000 | 0.000 | 0.000 | 0.000 | 0.000 |
| BRAMY2036387 | 0.000 | 0.000 | 0.000 | 0.000 | 0.000 | 13.490 | 0.000 | 0.000 | 0.000 | 0.000 | 0.000 | 0.000 | 0.000 |
| BRAMY2036396 | 0.000 | 0.000 | 0.000 | 0.000 | 0.000 | 100.000 | 0.000 | 0.000 | 0.000 | 0.000 | 0.000 | 0.000 | 0.000 |
| BRAMY2036567 | 0.000 | 18.633 | 0.000 | 26.525 | 0.000 | 22.594 | 7.474 | 17.148 | 0.000 | 7.626 | 0.000 | 0.000 | 0.000 |
| BRAMY2036699 | 0.000 | 0.000 | 0.000 | 0.000 | 0.000 | 100.000 | 0.000 | 0.000 | 0.000 | 0.000 | 0.000 | 0.000 | 0.000 |
| BRAMY2036913 | 0.000 | 0.000 | 0.000 | 0.000 | 0.000 | 100.000 | 0.000 | 0.000 | 0.000 | 0.000 | 0.000 | 0.000 | 0.000 |
| BRAMY2037823 | 0.000 | 0.000 | 0.000 | 21.055 | 0.000 | 29.890 | 29.664 | 0.000 | 0.000 | 0.000 | 0.000 | 0.000 | 0.000 |
| BRAMY2038100 | 0.000 | 0.000 | 0.000 | 0.000 | 0.000 | 100.000 | 0.000 | 0.000 | 0.000 | 0.000 | 0.000 | 0.000 | 0.000 |
| BRAMY2038484 | 0.000 | 0.000 | 0.000 | 0.000 | 0.000 | 100.000 | 0.000 | 0.000 | 0.000 | 0.000 | 0.000 | 0.000 | 0.000 |
| BRAMY2038846 | 0.000 | 0.000 | 0.000 | 0.000 | 0.000 | 47.355 | 0.000 | 0.000 | 0.000 | 0.000 | 0.000 | 0.000 | 0.000 |
| BRAMY2038904 | 0.000 | 0.000 | 0.000 | 0.000 | 0.000 | 60.652 | 0.000 | 0.000 | 0.000 | 0.000 | 0.000 | 0.000 | 0.000 |
| BRAMY2039872 | 0.000 | 49.422 | 0.000 | 14.071 | 0.000 | 19.976 | 0.000 | 0.000 | 0.000 | 0.000 | 0.000 | 0.000 | 0.000 |
| BRAMY2040478 | 0.000 | 0.000 | 0.000 | 0.000 | 0.000 | 100.000 | 0.000 | 0.000 | 0.000 | 0.000 | 0.000 | 0.000 | 0.000 |
| BRAMY2040592 | 0.000 | 0.000 | 0.000 | 0.000 | 12.582 | 7.017 | 3.482 | 0.000 | 0.000 | 7.105 | 0.000 | 0.000 | 0.000 |
| BRAMY2041261 | 0.000 | 0.000 | 0.000 | 0.000 | 0.000 | 100.000 | 0.000 | 0.000 | 0.000 | 0.000 | 0.000 | 0.000 | 0.000 |
| BRAMY2041378 | 0.000 | 0.000 | 0.000 | 0.000 | 0.000 | 100.000 | 0.000 | 0.000 | 0.000 | 0.000 | 0.000 | 0.000 | 0.000 |
| BRAMY2041542 | 0.000 | 0.000 | 0.000 | 0.000 | 0.000 | 100.000 | 0.000 | 0.000 | 0.000 | 0.000 | 0.000 | 0.000 | 0.000 |
| BRAMY2042612 | 0.000 | 0.000 | 0.000 | 0.000 | 0.000 | 100.000 | 0.000 | 0.000 | 0.000 | 0.000 | 0.000 | 0.000 | 0.000 |
| BRAMY2042641 | 0.000 | 0.000 | 0.000 | 0.000 | 0.000 | 100.000 | 0.000 | 0.000 | 0.000 | 0.000 | 0.000 | 0.000 | 0.000 |
| BRAMY2042760 | 0.000 | 0.000 | 0.000 | 0.000 | 0.000 | 100.000 | 0.000 | 0.000 | 0.000 | 0.000 | 0.000 | 0.000 | 0.000 |
| BRAMY2042918 | 0.000 | 0.000 | 0.000 | 0.000 | 0.000 | 100.000 | 0.000 | 0.000 | 0.000 | 0.000 | 0.000 | 0.000 | 0.000 |
| BRAMY2044078 | 24.301 | 0.000 | 5.447 | 6.224 | 0.000 | 8.836 | 0.000 | 0.000 | 0.000 | 8.947 | 0.000 | 0.000 | 19.449 |
| BRAMY2044246 | 0.000 | 0.000 | 0.000 | 0.000 | 0.000 | 100.000 | 0.000 | 0.000 | 0.000 | 0.000 | 0.000 | 0.000 | 0.000 |
| BRAMY2045036 | 0.000 | 0.000 | 0.000 | 0.000 | 0.000 | 100.000 | 0.000 | 0.000 | 0.000 | 0.000 | 0.000 | 0.000 | 0.000 |
| BRAMY2046478 | 0.000 | 0.000 | 0.000 | 0.000 | 0.000 | 100.000 | 0.000 | 0.000 | 0.000 | 0.000 | 0.000 | 0.000 | 0.000 |
| BRAMY2046742 | 0.000 | 0.000 | 0.000 | 0.000 | 0.000 | 100.000 | 0.000 | 0.000 | 0.000 | 0.000 | 0.000 | 0.000 | 0.000 |
| BRAMY2046989 | 0.000 | 0.000 | 0.000 | 0.000 | 0.000 | 100.000 | 0.000 | 0.000 | 0.000 | 0.000 | 0.000 | 0.000 | 0.000 |
| BRAMY2047169 | 0.000 | 0.000 | 0.000 | 0.000 | 0.000 | 100.000 | 0.000 | 0.000 | 0.000 | 0.000 | 0.000 | 0.000 | 0.000 |
| BRAMY2047420 | 0.000 | 0.000 | 0.000 | 0.719 | 0.000 | 2.041 | 0.000 | 0.000 | 0.000 | 0.000 | 3.770 | 0.000 | 0.000 |
| BRAMY2047676 | 0.000 | 0.000 | 0.000 | 0.000 | 0.000 | 100.000 | 0.000 | 0.000 | 0.000 | 0.000 | 0.000 | 0.000 | 0.000 |
| BRAMY2047746 | 0.000 | 0.000 | 0.000 | 0.000 | 0.000 | 100.000 | 0.000 | 0.000 | 0.000 | 0.000 | 0.000 | 0.000 | 0.000 |
| BRAMY2047751 | 0.000 | 0.000 | 0.000 | 0.000 | 0.000 | 100.000 | 0.000 | 0.000 | 0.000 | 0.000 | 0.000 | 0.000 | 0.000 |
| BRAMY2047765 | 0.000 | 0.000 | 0.000 | 0.000 | 0.000 | 100.000 | 0.000 | 0.000 | 0.000 | 0.000 | 0.000 | 0.000 | 0.000 |
| BRAMY2047884 | 0.000 | 0.000 | 0.000 | 0.000 | 0.000 | 100.000 | 0.000 | 0.000 | 0.000 | 0.000 | 0.000 | 0.000 | 0.000 |
| BRAMY3000206 | 0.000 | 0.000 | 0.000 | 0.000 | 0.000 | 100.000 | 0.000 | 0.000 | 0.000 | 0.000 | 0.000 | 0.000 | 0.000 |
| BRAMY3000213 | 0.000 | 0.000 | 0.000 | 0.000 | 0.000 | 100.000 | 0.000 | 0.000 | 0.000 | 0.000 | 0.000 | 0.000 | 0.000 |
| BRAMY3001401 | 0.000 | 0.000 | 0.000 | 0.000 | 0.000 | 100.000 | 0.000 | 0.000 | 0.000 | 0.000 | 0.000 | 0.000 | 0.000 |
| BRAMY3001794 | 0.000 | 0.000 | 0.000 | 0.000 | 0.000 | 100.000 | 0.000 | 0.000 | 0.000 | 0.000 | 0.000 | 0.000 | 0.000 |
| BRAMY3002312 | 0.000 | 0.000 | 0.000 | 0.000 | 0.000 | 100.000 | 0.000 | 0.000 | 0.000 | 0.000 | 0.000 | 0.000 | 0.000 |
| BRAMY3002620 | 0.000 | 0.000 | 0.000 | 41.329 | 0.000 | 58.671 | 0.000 | 0.000 | 0.000 | 0.000 | 0.000 | 0.000 | 0.000 |
| BRAMY3002803 | 0.000 | 0.000 | 0.000 | 0.000 | 0.000 | 14.538 | 14.428 | 0.000 | 0.000 | 44.161 | 0.000 | 0.000 | 0.000 |
| BRAMY3002805 | 0.000 | 0.000 | 0.000 | 0.000 | 0.000 | 100.000 | 0.000 | 0.000 | 0.000 | 0.000 | 0.000 | 0.000 | 0.000 |
| BRAMY3004224 | 0.000 | 0.000 | 0.000 | 0.000 | 0.000 | 33.278 | 33.027 | 0.000 | 0.000 | 33.695 | 0.000 | 0.000 | 0.000 |
| BRAMY3004672 | 0.000 | 0.000 | 0.000 | 0.000 | 0.000 | 100.000 | 0.000 | 0.000 | 0.000 | 0.000 | 0.000 | 0.000 | 0.000 |
| BRAMY3004900 | 0.000 | 0.000 | 0.000 | 0.000 | 0.000 | 100.000 | 0.000 | 0.000 | 0.000 | 0.000 | 0.000 | 0.000 | 0.000 |
| BRAMY3004919 | 0.000 | 0.000 | 0.000 | 0.000 | 0.000 | 100.000 | 0.000 | 0.000 | 0.000 | 0.000 | 0.000 | 0.000 | 0.000 |
| BRAMY3005091 | 0.000 | 47.846 | 0.000 | 13.622 | 0.000 | 19.339 | 19.193 | 0.000 | 0.000 | 0.000 | 0.000 | 0.000 | 0.000 |
| BRAMY3005932 | 0.000 | 0.000 | 0.000 | 41.329 | 0.000 | 58.671 | 0.000 | 0.000 | 0.000 | 0.000 | 0.000 | 0.000 | 0.000 |
| BRAMY3006297 | 0.000 | 0.000 | 0.000 | 0.000 | 0.000 | 100.000 | 0.000 | 0.000 | 0.000 | 0.000 | 0.000 | 0.000 | 0.000 |
| BRAMY3007206 | 0.000 | 0.000 | 0.000 | 0.000 | 0.000 | 100.000 | 0.000 | 0.000 | 0.000 | 0.000 | 0.000 | 0.000 | 0.000 |
| BRAMY3007609 | 0.000 | 0.000 | 0.000 | 0.000 | 0.000 | 100.000 | 0.000 | 0.000 | 0.000 | 0.000 | 0.000 | 0.000 | 0.000 |
| BRAMY3008466 | 0.000 | 0.000 | 0.000 | 0.000 | 0.000 | 100.000 | 0.000 | 0.000 | 0.000 | 0.000 | 0.000 | 0.000 | 0.000 |
| BRAMY3008505 | 0.000 | 0.000 | 0.000 | 0.000 | 0.000 | 100.000 | 0.000 | 0.000 | 0.000 | 0.000 | 0.000 | 0.000 | 0.000 |
| BRAMY3008650 | 0.000 | 0.000 | 0.000 | 0.000 | 0.000 | 100.000 | 0.000 | 0.000 | 0.000 | 0.000 | 0.000 | 0.000 | 0.000 |
| BRAMY3009811 | 0.000 | 0.000 | 0.000 | 0.000 | 0.000 | 33.057 | 0.000 | 0.000 | 0.000 | 66.943 | 0.000 | 0.000 | 0.000 |
| BRAMY3010411 | 0.000 | 0.000 | 0.000 | 0.000 | 0.000 | 100.000 | 0.000 | 0.000 | 0.000 | 0.000 | 0.000 | 0.000 | 0.000 |
| BRAMY4000095 | 0.000 | 0.000 | 0.000 | 0.000 | 0.000 | 14.721 | 0.000 | 33.520 | 51.759 | 0.000 | 0.000 | 0.000 | 0.000 |
| BRAMY4000229 | 0.000 | 0.000 | 0.000 | 25.113 | 0.000 | 35.650 | 0.000 | 0.000 | 0.000 | 0.000 | 0.000 | 0.000 | 39.237 |
| BRAMY4000277 | 0.000 | 0.000 | 0.000 | 0.000 | 0.000 | 100.000 | 0.000 | 0.000 | 0.000 | 0.000 | 0.000 | 0.000 | 0.000 |
| BRASW1000125 | 0.000 | 0.000 | 0.000 | 0.000 | 0.000 | 0.000 | 0.000 | 0.000 | 0.000 | 0.000 | 0.000 | 0.946 | 0.000 |
| BRAWH1000127 | 0.000 | 7.969 | 3.971 | 4.538 | 0.000 | 9.663 | 15.983 | 0.000 | 0.000 | 3.261 | 35.693 | 0.000 | 7.090 |
| BRAWH2001395 | 0.000 | 0.000 | 0.000 | 3.580 | 3.037 | 2.541 | 14.290 | 5.785 | 10.422 | 22.297 | 12.514 | 4.546 | 11.652 |
| BRAWH2001671 | 0.000 | 0.000 | 0.000 | 8.097 | 0.000 | 0.000 | 7.605 | 0.000 | 0.000 | 11.638 | 0.000 | 0.000 | 12.650 |
| BRAWH2001940 | 0.000 | 0.000 | 0.000 | 0.000 | 0.000 | 0.000 | 37.398 | 0.000 | 0.000 | 38.155 | 0.000 | 0.000 | 0.000 |
| BRAWH2001973 | 0.000 | 0.000 | 0.000 | 0.000 | 0.000 | 0.000 | 37.398 | 0.000 | 0.000 | 38.155 | 0.000 | 0.000 | 0.000 |
| BRAWH2002560 | 11.923 | 24.132 | 24.052 | 0.000 | 0.000 | 6.503 | 6.454 | 0.000 | 0.000 | 3.292 | 12.010 | 11.635 | 0.000 |
| BRAWH2002761 | 0.000 | 0.000 | 0.000 | 0.000 | 0.000 | 0.000 | 100.000 | 0.000 | 0.000 | 0.000 | 0.000 | 0.000 | 0.000 |
| BRAWH2005315 | 0.000 | 0.000 | 0.000 | 0.000 | 0.000 | 0.000 | 100.000 | 0.000 | 0.000 | 0.000 | 0.000 | 0.000 | 0.000 |
| BRAWH2007658 | 0.000 | 0.000 | 0.000 | 0.000 | 0.000 | 0.000 | 58.101 | 0.000 | 0.000 | 0.000 | 0.000 | 41.899 | 0.000 |
| BRAWH2010000 | 0.000 | 0.000 | 0.000 | 0.000 | 0.000 | 0.000 | 18.745 | 43.006 | 0.000 | 38.249 | 0.000 | 0.000 | 0.000 |
| BRAWH2010084 | 0.000 | 0.000 | 0.000 | 0.000 | 0.000 | 0.000 | 100.000 | 0.000 | 0.000 | 0.000 | 0.000 | 0.000 | 0.000 |
| BRAWH2010536 | 0.000 | 0.000 | 0.000 | 0.000 | 0.000 | 0.000 | 14.718 | 0.000 | 0.000 | 0.000 | 0.000 | 0.000 | 0.000 |
| BRAWH2012162 | 0.000 | 0.000 | 0.000 | 0.000 | 0.000 | 0.000 | 36.060 | 0.000 | 0.000 | 0.000 | 0.000 | 0.000 | 0.000 |
| BRAWH2012326 | 0.000 | 0.000 | 0.000 | 0.000 | 0.000 | 0.000 | 100.000 | 0.000 | 0.000 | 0.000 | 0.000 | 0.000 | 0.000 |
| BRAWH2013294 | 0.000 | 0.000 | 0.000 | 13.997 | 0.000 | 0.000 | 39.442 | 0.000 | 0.000 | 10.060 | 0.000 | 0.000 | 0.000 |
| BRAWH2013871 | 0.000 | 0.000 | 0.000 | 0.000 | 0.000 | 0.000 | 37.485 | 0.000 | 0.000 | 0.000 | 0.000 | 0.000 | 0.000 |
| BRAWH2014414 | 0.000 | 0.000 | 0.000 | 0.000 | 0.000 | 54.002 | 17.865 | 0.000 | 0.000 | 18.227 | 0.000 | 0.000 | 9.906 |
| BRAWH2014645 | 0.000 | 2.635 | 6.566 | 3.751 | 0.000 | 2.130 | 4.228 | 0.000 | 3.745 | 1.078 | 0.000 | 0.000 | 1.172 |
| BRAWH2014662 | 0.000 | 0.000 | 0.000 | 11.016 | 0.000 | 0.000 | 15.521 | 0.000 | 0.000 | 0.000 | 0.000 | 0.000 | 17.212 |
| BRAWH2014876 | 0.000 | 0.000 | 0.000 | 7.433 | 0.000 | 0.000 | 10.473 | 24.026 | 0.000 | 0.000 | 0.000 | 0.000 | 58.068 |
| BRAWH2014954 | 0.000 | 0.000 | 0.000 | 41.512 | 0.000 | 0.000 | 58.488 | 0.000 | 0.000 | 0.000 | 0.000 | 0.000 | 0.000 |
| BRAWH2016221 | 0.000 | 0.000 | 0.000 | 0.000 | 0.000 | 0.000 | 47.417 | 0.000 | 0.000 | 0.000 | 0.000 | 0.000 | 52.583 |
| BRAWH2016439 | 0.000 | 0.000 | 0.000 | 0.000 | 0.000 | 0.000 | 100.000 | 0.000 | 0.000 | 0.000 | 0.000 | 0.000 | 0.000 |
| BRAWH2016702 | 0.000 | 0.000 | 0.000 | 26.193 | 0.000 | 0.000 | 73.807 | 0.000 | 0.000 | 0.000 | 0.000 | 0.000 | 0.000 |
| BRAWH2016724 | 64.881 | 0.000 | 0.000 | 0.000 | 0.000 | 0.000 | 35.119 | 0.000 | 0.000 | 0.000 | 0.000 | 0.000 | 0.000 |
| BRAWH3000078 | 0.000 | 0.000 | 0.000 | 0.000 | 0.000 | 0.000 | 100.000 | 0.000 | 0.000 | 0.000 | 0.000 | 0.000 | 0.000 |
| BRAWH3000100 | 0.000 | 0.000 | 0.000 | 0.000 | 0.000 | 0.000 | 100.000 | 0.000 | 0.000 | 0.000 | 0.000 | 0.000 | 0.000 |
| BRAWH3000314 | 0.000 | 0.000 | 0.000 | 0.000 | 0.000 | 0.000 | 71.553 | 0.000 | 0.000 | 0.000 | 0.000 | 0.000 | 0.000 |
| BRAWH3000491 | 0.000 | 0.000 | 0.000 | 0.000 | 0.000 | 0.000 | 100.000 | 0.000 | 0.000 | 0.000 | 0.000 | 0.000 | 0.000 |
| BRAWH3001326 | 0.000 | 0.000 | 0.000 | 0.000 | 0.000 | 0.000 | 45.606 | 0.000 | 0.000 | 0.000 | 0.000 | 0.000 | 0.000 |
| BRAWH3001475 | 0.000 | 0.000 | 0.000 | 0.000 | 0.000 | 0.000 | 100.000 | 0.000 | 0.000 | 0.000 | 0.000 | 0.000 | 0.000 |
| BRAWH3001891 | 0.000 | 0.000 | 0.000 | 36.772 | 0.000 | 17.401 | 34.539 | 0.000 | 0.000 | 0.000 | 0.000 | 0.000 | 0.000 |
| BRAWH3002574 | 0.000 | 0.000 | 8.213 | 0.000 | 0.000 | 0.000 | 13.222 | 0.000 | 0.000 | 0.000 | 0.000 | 0.000 | 0.000 |
| BRAWH3002600 | 0.000 | 0.000 | 0.000 | 26.120 | 0.000 | 37.080 | 36.800 | 0.000 | 0.000 | 0.000 | 0.000 | 0.000 | 0.000 |
| BRAWH3002819 | 0.000 | 0.000 | 0.000 | 0.000 | 0.000 | 0.000 | 100.000 | 0.000 | 0.000 | 0.000 | 0.000 | 0.000 | 0.000 |
| BRAWH3002821 | 0.000 | 0.000 | 27.272 | 0.000 | 0.000 | 0.000 | 21.953 | 0.000 | 0.000 | 0.000 | 0.000 | 0.000 | 0.000 |
| BRAWH3003522 | 0.000 | 0.000 | 0.000 | 0.000 | 0.000 | 0.000 | 100.000 | 0.000 | 0.000 | 0.000 | 0.000 | 0.000 | 0.000 |
| BRAWH3003555 | 0.000 | 0.000 | 0.000 | 5.405 | 0.000 | 0.000 | 15.229 | 0.000 | 0.000 | 0.000 | 0.000 | 0.000 | 8.444 |
| BRAWH3003727 | 0.000 | 0.000 | 12.492 | 21.411 | 0.000 | 0.000 | 10.055 | 0.000 | 0.000 | 10.259 | 0.000 | 0.000 | 0.000 |
| BRAWH3003801 | 0.000 | 0.000 | 0.000 | 0.000 | 0.000 | 0.000 | 100.000 | 0.000 | 0.000 | 0.000 | 0.000 | 0.000 | 0.000 |
| BRAWH3003992 | 0.000 | 0.000 | 36.036 | 0.000 | 0.000 | 0.000 | 29.008 | 0.000 | 0.000 | 0.000 | 0.000 | 0.000 | 0.000 |
| BRAWH3004453 | 0.000 | 0.000 | 0.000 | 0.000 | 0.000 | 0.000 | 100.000 | 0.000 | 0.000 | 0.000 | 0.000 | 0.000 | 0.000 |
| BRAWH3004666 | 0.000 | 0.000 | 0.000 | 0.000 | 0.000 | 0.000 | 49.499 | 0.000 | 0.000 | 50.501 | 0.000 | 0.000 | 0.000 |
| BRAWH3005132 | 0.000 | 0.000 | 0.000 | 0.000 | 0.000 | 50.189 | 49.811 | 0.000 | 0.000 | 0.000 | 0.000 | 0.000 | 0.000 |
| BRAWH3005422 | 0.000 | 0.000 | 0.000 | 0.000 | 0.000 | 0.000 | 100.000 | 0.000 | 0.000 | 0.000 | 0.000 | 0.000 | 0.000 |
| BRAWH3005912 | 0.000 | 0.000 | 0.000 | 0.000 | 0.000 | 0.000 | 100.000 | 0.000 | 0.000 | 0.000 | 0.000 | 0.000 | 0.000 |
| BRAWH3005981 | 0.000 | 0.000 | 0.000 | 0.000 | 0.000 | 0.000 | 29.324 | 0.000 | 0.000 | 0.000 | 0.000 | 0.000 | 0.000 |
| BRAWH3006548 | 0.000 | 0.000 | 0.000 | 0.000 | 0.000 | 0.000 | 71.018 | 0.000 | 0.000 | 28.982 | 0.000 | 0.000 | 0.000 |
| BRAWH3006792 | 0.000 | 0.000 | 0.000 | 0.000 | 0.000 | 0.000 | 49.499 | 0.000 | 0.000 | 50.501 | 0.000 | 0.000 | 0.000 |
| BRAWH3007221 | 0.000 | 0.000 | 0.000 | 0.000 | 0.000 | 0.000 | 100.000 | 0.000 | 0.000 | 0.000 | 0.000 | 0.000 | 0.000 |
| BRAWH3007506 | 0.000 | 0.000 | 0.000 | 0.000 | 0.000 | 0.000 | 100.000 | 0.000 | 0.000 | 0.000 | 0.000 | 0.000 | 0.000 |
| BRAWH3007592 | 0.000 | 0.000 | 6.683 | 6.364 | 0.000 | 1.807 | 8.966 | 0.000 | 0.000 | 3.659 | 0.000 | 0.000 | 0.000 |
| BRAWH3007726 | 0.000 | 0.000 | 0.000 | 0.000 | 0.000 | 0.000 | 54.530 | 0.000 | 0.000 | 0.000 | 0.000 | 0.000 | 0.000 |
| BRAWH3007783 | 0.000 | 0.000 | 0.000 | 0.000 | 0.000 | 0.000 | 100.000 | 0.000 | 0.000 | 0.000 | 0.000 | 0.000 | 0.000 |
| BRAWH3008341 | 0.000 | 0.000 | 0.000 | 0.000 | 0.000 | 0.000 | 100.000 | 0.000 | 0.000 | 0.000 | 0.000 | 0.000 | 0.000 |
| BRAWH3008697 | 0.000 | 0.000 | 0.000 | 0.000 | 0.000 | 0.000 | 100.000 | 0.000 | 0.000 | 0.000 | 0.000 | 0.000 | 0.000 |
| BRAWH3008931 | 0.000 | 0.000 | 4.303 | 4.916 | 0.000 | 6.980 | 3.463 | 15.892 | 0.000 | 10.601 | 12.891 | 0.000 | 0.000 |
| BRAWH3009297 | 0.000 | 0.000 | 0.000 | 41.512 | 0.000 | 0.000 | 58.488 | 0.000 | 0.000 | 0.000 | 0.000 | 0.000 | 0.000 |
| BRCAN2002562 | 0.000 | 0.000 | 0.000 | 0.000 | 0.000 | 0.000 | 0.000 | 100.000 | 0.000 | 0.000 | 0.000 | 0.000 | 0.000 |
| BRCAN2002856 | 0.000 | 0.000 | 0.000 | 0.000 | 0.000 | 0.000 | 0.000 | 39.306 | 60.694 | 0.000 | 0.000 | 0.000 | 0.000 |
| BRCAN2002944 | 0.000 | 0.000 | 0.000 | 0.000 | 0.000 | 0.000 | 0.000 | 73.343 | 0.000 | 0.000 | 0.000 | 0.000 | 0.000 |
| BRCAN2002948 | 0.000 | 0.000 | 0.000 | 0.000 | 0.000 | 0.000 | 0.000 | 100.000 | 0.000 | 0.000 | 0.000 | 0.000 | 0.000 |
| BRCAN2003703 | 0.000 | 0.000 | 0.000 | 0.000 | 0.000 | 0.000 | 0.000 | 100.000 | 0.000 | 0.000 | 0.000 | 0.000 | 0.000 |
| BRCAN2003746 | 0.000 | 0.000 | 0.000 | 0.000 | 0.000 | 0.000 | 0.000 | 100.000 | 0.000 | 0.000 | 0.000 | 0.000 | 0.000 |
| BRCAN2003987 | 0.000 | 0.000 | 0.000 | 0.000 | 0.000 | 0.000 | 0.000 | 100.000 | 0.000 | 0.000 | 0.000 | 0.000 | 0.000 |
| BRCAN2004355 | 0.000 | 0.000 | 0.000 | 0.000 | 0.000 | 0.000 | 0.000 | 100.000 | 0.000 | 0.000 | 0.000 | 0.000 | 0.000 |
| BRCAN2005436 | 0.000 | 0.000 | 0.000 | 0.000 | 0.000 | 0.000 | 0.000 | 100.000 | 0.000 | 0.000 | 0.000 | 0.000 | 0.000 |
| BRCAN2006063 | 0.000 | 0.000 | 0.000 | 0.000 | 0.000 | 0.000 | 0.000 | 100.000 | 0.000 | 0.000 | 0.000 | 0.000 | 0.000 |
| BRCAN2006290 | 0.000 | 0.000 | 0.000 | 0.000 | 0.000 | 0.000 | 0.000 | 100.000 | 0.000 | 0.000 | 0.000 | 0.000 | 0.000 |
| BRCAN2006297 | 0.000 | 0.000 | 0.000 | 0.000 | 0.000 | 0.000 | 0.000 | 33.031 | 51.004 | 0.000 | 0.000 | 0.000 | 15.966 |
| BRCAN2006450 | 0.000 | 0.000 | 0.000 | 0.000 | 0.000 | 0.000 | 0.000 | 100.000 | 0.000 | 0.000 | 0.000 | 0.000 | 0.000 |
| BRCAN2007144 | 0.000 | 0.000 | 0.000 | 0.000 | 0.000 | 0.000 | 0.000 | 100.000 | 0.000 | 0.000 | 0.000 | 0.000 | 0.000 |
| BRCAN2007409 | 0.000 | 0.000 | 3.727 | 0.000 | 0.000 | 0.000 | 0.000 | 6.883 | 10.629 | 0.000 | 0.000 | 0.000 | 0.000 |
| BRCAN2007426 | 0.000 | 0.000 | 0.000 | 0.000 | 0.000 | 0.000 | 0.000 | 100.000 | 0.000 | 0.000 | 0.000 | 0.000 | 0.000 |
| BRCAN2008528 | 0.000 | 0.000 | 0.000 | 0.000 | 0.000 | 0.000 | 0.000 | 100.000 | 0.000 | 0.000 | 0.000 | 0.000 | 0.000 |
| BRCAN2009203 | 0.000 | 0.000 | 0.000 | 0.000 | 0.000 | 0.000 | 0.000 | 100.000 | 0.000 | 0.000 | 0.000 | 0.000 | 0.000 |
| BRCAN2009432 | 0.000 | 0.000 | 0.000 | 1.869 | 0.000 | 0.000 | 0.000 | 3.021 | 0.000 | 0.000 | 0.000 | 0.000 | 0.000 |
| BRCAN2010376 | 0.000 | 0.000 | 0.000 | 23.627 | 0.000 | 0.000 | 0.000 | 76.373 | 0.000 | 0.000 | 0.000 | 0.000 | 0.000 |
| BRCAN2011254 | 0.000 | 0.000 | 0.000 | 0.000 | 0.000 | 0.000 | 0.000 | 100.000 | 0.000 | 0.000 | 0.000 | 0.000 | 0.000 |
| BRCAN2011602 | 0.000 | 0.000 | 0.000 | 0.000 | 0.000 | 0.000 | 0.000 | 100.000 | 0.000 | 0.000 | 0.000 | 0.000 | 0.000 |
| BRCAN2012355 | 0.000 | 0.000 | 0.000 | 0.000 | 0.000 | 0.000 | 0.000 | 100.000 | 0.000 | 0.000 | 0.000 | 0.000 | 0.000 |
| BRCAN2012481 | 0.000 | 0.000 | 0.000 | 0.000 | 0.000 | 0.000 | 0.000 | 100.000 | 0.000 | 0.000 | 0.000 | 0.000 | 0.000 |
| BRCAN2013655 | 0.000 | 0.000 | 0.000 | 0.000 | 0.000 | 0.000 | 0.000 | 100.000 | 0.000 | 0.000 | 0.000 | 0.000 | 0.000 |
| BRCAN2013660 | 0.000 | 0.000 | 0.000 | 0.000 | 0.000 | 0.000 | 0.000 | 100.000 | 0.000 | 0.000 | 0.000 | 0.000 | 0.000 |
| BRCAN2014143 | 0.000 | 0.000 | 0.000 | 0.000 | 0.000 | 0.000 | 0.000 | 100.000 | 0.000 | 0.000 | 0.000 | 0.000 | 0.000 |
| BRCAN2014602 | 0.000 | 0.000 | 0.000 | 0.000 | 0.000 | 0.000 | 0.000 | 100.000 | 0.000 | 0.000 | 0.000 | 0.000 | 0.000 |
| BRCAN2014881 | 0.000 | 0.000 | 0.000 | 0.000 | 0.000 | 30.516 | 0.000 | 69.484 | 0.000 | 0.000 | 0.000 | 0.000 | 0.000 |
| BRCAN2015371 | 0.000 | 0.000 | 0.000 | 15.560 | 0.000 | 0.000 | 0.000 | 25.149 | 0.000 | 0.000 | 0.000 | 0.000 | 0.000 |
| BRCAN2015464 | 0.000 | 0.000 | 0.000 | 0.000 | 0.000 | 0.000 | 0.000 | 100.000 | 0.000 | 0.000 | 0.000 | 0.000 | 0.000 |
| BRCAN2016433 | 0.000 | 0.000 | 0.000 | 0.000 | 0.000 | 0.000 | 0.000 | 100.000 | 0.000 | 0.000 | 0.000 | 0.000 | 0.000 |
| BRCAN2016619 | 0.000 | 0.000 | 0.000 | 0.000 | 0.000 | 0.000 | 0.000 | 30.224 | 0.000 | 0.000 | 0.000 | 0.000 | 0.000 |
| BRCAN2017442 | 0.000 | 0.000 | 0.000 | 0.000 | 0.000 | 0.000 | 0.000 | 100.000 | 0.000 | 0.000 | 0.000 | 0.000 | 0.000 |
| BRCAN2017717 | 0.000 | 26.519 | 0.000 | 0.000 | 0.000 | 10.719 | 0.000 | 24.406 | 0.000 | 0.000 | 0.000 | 38.357 | 0.000 |
| BRCAN2017905 | 0.000 | 0.000 | 0.000 | 0.000 | 0.000 | 0.000 | 0.000 | 100.000 | 0.000 | 0.000 | 0.000 | 0.000 | 0.000 |
| BRCAN2018935 | 0.000 | 0.000 | 0.000 | 0.000 | 0.000 | 0.000 | 0.000 | 20.928 | 0.000 | 0.000 | 0.000 | 0.000 | 0.000 |
| BRCAN2019387 | 0.000 | 0.000 | 0.000 | 0.000 | 0.000 | 0.000 | 0.000 | 100.000 | 0.000 | 0.000 | 0.000 | 0.000 | 0.000 |
| BRCAN2020710 | 0.000 | 0.000 | 0.000 | 7.714 | 0.000 | 0.000 | 0.000 | 49.868 | 0.000 | 22.176 | 0.000 | 0.000 | 0.000 |
| BRCAN2021028 | 0.000 | 0.000 | 0.000 | 0.000 | 0.000 | 0.000 | 0.000 | 24.138 | 37.272 | 0.000 | 0.000 | 0.000 | 0.000 |
| BRCAN2024451 | 0.000 | 0.000 | 0.000 | 0.000 | 0.000 | 0.000 | 0.000 | 100.000 | 0.000 | 0.000 | 0.000 | 0.000 | 0.000 |
| BRCAN2024563 | 0.000 | 0.000 | 0.000 | 0.000 | 0.000 | 0.000 | 0.000 | 100.000 | 0.000 | 0.000 | 0.000 | 0.000 | 0.000 |
| BRCAN2025712 | 0.000 | 0.000 | 0.000 | 0.000 | 0.000 | 0.000 | 0.000 | 100.000 | 0.000 | 0.000 | 0.000 | 0.000 | 0.000 |
| BRCAN2028355 | 0.000 | 0.000 | 0.000 | 0.000 | 0.000 | 0.000 | 0.000 | 3.389 | 0.000 | 1.507 | 0.000 | 0.000 | 0.000 |
| BRCOC2000670 | 0.000 | 0.000 | 0.000 | 0.000 | 0.000 | 22.144 | 0.000 | 0.000 | 77.856 | 0.000 | 0.000 | 0.000 | 0.000 |
| BRCOC2003213 | 0.000 | 0.000 | 0.000 | 7.368 | 0.000 | 0.000 | 10.381 | 0.000 | 36.777 | 0.000 | 0.000 | 0.000 | 0.000 |
| BRCOC2007034 | 0.000 | 0.000 | 0.000 | 16.691 | 0.000 | 0.000 | 0.000 | 0.000 | 83.309 | 0.000 | 0.000 | 0.000 | 0.000 |
| BRCOC2014033 | 0.000 | 0.000 | 0.000 | 0.000 | 0.000 | 0.000 | 15.633 | 0.000 | 55.382 | 15.950 | 0.000 | 0.000 | 0.000 |
| BRCOC2016525 | 0.000 | 0.000 | 0.000 | 0.000 | 0.000 | 0.000 | 0.000 | 0.000 | 100.000 | 0.000 | 0.000 | 0.000 | 0.000 |
| BRCOC2019934 | 0.000 | 0.000 | 0.000 | 0.000 | 0.000 | 0.000 | 0.000 | 0.000 | 100.000 | 0.000 | 0.000 | 0.000 | 0.000 |
| BRCOC2020142 | 0.000 | 0.000 | 0.000 | 0.000 | 0.000 | 0.000 | 22.014 | 0.000 | 77.986 | 0.000 | 0.000 | 0.000 | 0.000 |
| BRHIP2000691 | 0.000 | 0.000 | 0.000 | 0.000 | 0.000 | 0.000 | 0.000 | 0.000 | 0.000 | 100.000 | 0.000 | 0.000 | 0.000 |
| BRHIP2000819 | 0.000 | 0.000 | 0.000 | 0.000 | 0.000 | 0.000 | 0.000 | 0.000 | 0.000 | 2.204 | 0.000 | 0.000 | 4.791 |
| BRHIP2000826 | 0.000 | 0.000 | 0.000 | 0.000 | 0.000 | 0.000 | 0.000 | 0.000 | 0.000 | 100.000 | 0.000 | 0.000 | 0.000 |
| BRHIP2000920 | 0.000 | 0.000 | 0.000 | 25.999 | 0.000 | 0.000 | 36.630 | 0.000 | 0.000 | 37.371 | 0.000 | 0.000 | 0.000 |
| BRHIP2001074 | 0.000 | 0.000 | 0.000 | 0.000 | 0.000 | 0.000 | 0.000 | 0.000 | 0.000 | 39.976 | 0.000 | 0.000 | 0.000 |
| BRHIP2001805 | 0.000 | 0.000 | 0.000 | 0.000 | 0.000 | 0.000 | 0.000 | 0.000 | 0.000 | 14.757 | 0.000 | 0.000 | 0.000 |
| BRHIP2001927 | 0.000 | 0.000 | 0.000 | 0.000 | 0.000 | 0.000 | 0.000 | 0.000 | 0.000 | 100.000 | 0.000 | 0.000 | 0.000 |
| BRHIP2002122 | 0.000 | 0.000 | 0.000 | 0.000 | 0.000 | 0.000 | 0.000 | 0.000 | 0.000 | 100.000 | 0.000 | 0.000 | 0.000 |
| BRHIP2002172 | 0.000 | 0.000 | 0.000 | 0.000 | 0.000 | 0.000 | 0.000 | 0.000 | 0.000 | 100.000 | 0.000 | 0.000 | 0.000 |
| BRHIP2002346 | 0.000 | 0.000 | 0.000 | 0.000 | 0.000 | 0.000 | 0.000 | 0.000 | 0.000 | 100.000 | 0.000 | 0.000 | 0.000 |
| BRHIP2003242 | 0.000 | 0.000 | 0.000 | 0.000 | 0.000 | 0.000 | 0.000 | 0.000 | 0.000 | 100.000 | 0.000 | 0.000 | 0.000 |
| BRHIP2003786 | 0.000 | 0.000 | 0.000 | 0.000 | 0.000 | 0.000 | 0.000 | 0.000 | 0.000 | 100.000 | 0.000 | 0.000 | 0.000 |
| BRHIP2003917 | 0.000 | 0.000 | 0.000 | 0.000 | 0.000 | 0.000 | 0.000 | 0.000 | 0.000 | 11.981 | 0.000 | 0.000 | 0.000 |
| BRHIP2004312 | 0.000 | 0.000 | 0.000 | 0.000 | 0.000 | 0.000 | 0.000 | 0.000 | 0.000 | 100.000 | 0.000 | 0.000 | 0.000 |
| BRHIP2004359 | 0.000 | 0.000 | 0.000 | 34.560 | 0.000 | 0.000 | 0.000 | 0.000 | 0.000 | 24.839 | 0.000 | 0.000 | 0.000 |
| BRHIP2004814 | 0.000 | 0.000 | 0.000 | 0.000 | 0.000 | 0.000 | 0.000 | 0.000 | 0.000 | 35.391 | 0.000 | 0.000 | 0.000 |
| BRHIP2004883 | 0.000 | 0.000 | 0.000 | 0.000 | 0.000 | 0.000 | 0.000 | 0.000 | 0.000 | 100.000 | 0.000 | 0.000 | 0.000 |
| BRHIP2005236 | 0.000 | 0.000 | 0.000 | 0.000 | 0.000 | 0.000 | 0.000 | 0.000 | 0.000 | 100.000 | 0.000 | 0.000 | 0.000 |
| BRHIP2005354 | 0.000 | 0.000 | 0.000 | 0.000 | 0.000 | 0.000 | 0.000 | 0.000 | 0.000 | 100.000 | 0.000 | 0.000 | 0.000 |
| BRHIP2005600 | 0.000 | 0.000 | 0.000 | 0.000 | 0.000 | 0.000 | 0.000 | 0.000 | 0.000 | 100.000 | 0.000 | 0.000 | 0.000 |
| BRHIP2005719 | 0.000 | 0.000 | 0.000 | 0.000 | 0.000 | 0.000 | 49.499 | 0.000 | 0.000 | 50.501 | 0.000 | 0.000 | 0.000 |
| BRHIP2005752 | 0.000 | 0.000 | 0.000 | 41.027 | 0.000 | 0.000 | 0.000 | 0.000 | 0.000 | 58.973 | 0.000 | 0.000 | 0.000 |
| BRHIP2005932 | 0.000 | 0.000 | 0.000 | 0.000 | 0.000 | 0.000 | 0.000 | 0.000 | 0.000 | 100.000 | 0.000 | 0.000 | 0.000 |
| BRHIP2006800 | 0.000 | 0.000 | 0.000 | 0.000 | 0.000 | 0.000 | 0.000 | 0.000 | 0.000 | 100.000 | 0.000 | 0.000 | 0.000 |
| BRHIP2007616 | 0.000 | 0.000 | 0.000 | 0.000 | 0.000 | 0.000 | 0.000 | 0.000 | 0.000 | 100.000 | 0.000 | 0.000 | 0.000 |
| BRHIP2007741 | 3.207 | 4.327 | 4.313 | 3.696 | 0.000 | 8.745 | 6.943 | 0.000 | 0.000 | 3.542 | 0.000 | 0.000 | 5.775 |
| BRHIP2009340 | 0.000 | 0.000 | 0.000 | 0.000 | 0.000 | 0.000 | 0.000 | 0.000 | 0.000 | 100.000 | 0.000 | 0.000 | 0.000 |
| BRHIP2009414 | 0.000 | 0.000 | 0.000 | 0.000 | 0.000 | 0.000 | 0.000 | 0.000 | 0.000 | 100.000 | 0.000 | 0.000 | 0.000 |
| BRHIP2009474 | 0.000 | 0.000 | 0.000 | 0.000 | 0.000 | 0.000 | 0.000 | 0.000 | 0.000 | 47.917 | 0.000 | 0.000 | 52.083 |
| BRHIP2013699 | 39.421 | 0.000 | 0.000 | 15.145 | 0.000 | 0.000 | 0.000 | 0.000 | 0.000 | 21.770 | 0.000 | 0.000 | 23.663 |
| BRHIP2014228 | 0.000 | 0.000 | 0.000 | 10.447 | 0.000 | 0.000 | 29.439 | 0.000 | 0.000 | 15.017 | 0.000 | 0.000 | 0.000 |
| BRHIP2021615 | 0.000 | 0.000 | 0.000 | 0.000 | 0.000 | 0.000 | 0.000 | 0.000 | 0.000 | 100.000 | 0.000 | 0.000 | 0.000 |
| BRHIP2022221 | 0.000 | 0.000 | 0.000 | 0.000 | 0.000 | 0.000 | 0.000 | 0.000 | 0.000 | 47.917 | 0.000 | 0.000 | 52.083 |
| BRHIP2024146 | 0.000 | 0.000 | 0.000 | 0.864 | 12.091 | 2.759 | 3.042 | 11.166 | 16.165 | 5.897 | 10.190 | 6.581 | 7.759 |
| BRHIP2024165 | 0.000 | 0.000 | 0.000 | 0.000 | 0.000 | 0.000 | 0.000 | 0.000 | 0.000 | 100.000 | 0.000 | 0.000 | 0.000 |
| BRHIP2026061 | 0.000 | 0.000 | 37.989 | 0.000 | 0.000 | 30.812 | 0.000 | 0.000 | 0.000 | 31.199 | 0.000 | 0.000 | 0.000 |
| BRHIP2026288 | 0.000 | 0.000 | 0.000 | 0.000 | 77.982 | 0.000 | 0.000 | 0.000 | 0.000 | 22.018 | 0.000 | 0.000 | 0.000 |
| BRHIP2029176 | 0.000 | 0.000 | 0.000 | 0.000 | 0.000 | 0.000 | 0.000 | 0.000 | 0.000 | 100.000 | 0.000 | 0.000 | 0.000 |
| BRHIP2029393 | 0.000 | 0.000 | 0.000 | 0.000 | 0.000 | 0.000 | 0.000 | 0.000 | 0.000 | 100.000 | 0.000 | 0.000 | 0.000 |
| BRHIP3000339 | 0.000 | 0.000 | 0.000 | 3.580 | 3.037 | 2.541 | 14.290 | 5.785 | 10.422 | 22.297 | 12.514 | 4.546 | 11.652 |
| BRHIP3000526 | 0.000 | 0.000 | 0.000 | 0.000 | 0.000 | 0.000 | 0.000 | 0.000 | 0.000 | 100.000 | 0.000 | 0.000 | 0.000 |
| BRHIP3001283 | 0.000 | 0.000 | 0.000 | 0.000 | 0.000 | 49.688 | 0.000 | 0.000 | 0.000 | 50.312 | 0.000 | 0.000 | 0.000 |
| BRHIP3006683 | 0.000 | 0.000 | 0.000 | 0.000 | 0.000 | 0.000 | 24.100 | 0.000 | 0.000 | 49.175 | 0.000 | 0.000 | 26.726 |
| BRHIP3007483 | 0.000 | 0.000 | 0.000 | 0.000 | 0.000 | 0.000 | 0.000 | 0.000 | 0.000 | 100.000 | 0.000 | 0.000 | 0.000 |
| BRHIP3007586 | 6.375 | 0.000 | 0.000 | 7.348 | 0.000 | 3.477 | 17.255 | 0.000 | 0.000 | 24.645 | 0.000 | 0.000 | 11.481 |
| BRHIP3008183 | 0.000 | 0.000 | 0.000 | 0.000 | 0.000 | 0.000 | 0.000 | 0.000 | 0.000 | 100.000 | 0.000 | 0.000 | 0.000 |
| BRHIP3008313 | 0.000 | 0.000 | 0.000 | 20.257 | 0.000 | 0.000 | 0.000 | 0.000 | 0.000 | 29.118 | 0.000 | 0.000 | 0.000 |
| BRHIP3008344 | 0.000 | 0.000 | 0.000 | 0.000 | 0.000 | 0.000 | 0.000 | 0.000 | 0.000 | 46.104 | 0.000 | 0.000 | 0.000 |
| BRHIP3008405 | 0.000 | 0.000 | 0.000 | 12.487 | 0.000 | 0.000 | 35.187 | 0.000 | 0.000 | 17.949 | 0.000 | 0.000 | 0.000 |
| BRHIP3008565 | 0.000 | 0.000 | 0.000 | 0.000 | 0.000 | 0.000 | 0.000 | 0.000 | 0.000 | 46.104 | 0.000 | 0.000 | 0.000 |
| BRHIP3008598 | 0.000 | 0.000 | 0.000 | 0.000 | 0.000 | 0.000 | 0.000 | 0.000 | 0.000 | 100.000 | 0.000 | 0.000 | 0.000 |
| BRHIP3008997 | 0.000 | 0.000 | 0.000 | 0.000 | 0.000 | 0.000 | 0.000 | 0.000 | 0.000 | 100.000 | 0.000 | 0.000 | 0.000 |
| BRHIP3009099 | 64.423 | 0.000 | 0.000 | 0.000 | 0.000 | 0.000 | 0.000 | 0.000 | 0.000 | 35.577 | 0.000 | 0.000 | 0.000 |
| BRHIP3009448 | 0.000 | 0.000 | 0.000 | 0.000 | 0.000 | 0.000 | 0.000 | 0.000 | 0.000 | 100.000 | 0.000 | 0.000 | 0.000 |
| BRHIP3011241 | 0.000 | 0.000 | 0.000 | 0.000 | 0.000 | 0.000 | 0.000 | 0.000 | 0.000 | 100.000 | 0.000 | 0.000 | 0.000 |
| BRHIP3013765 | 0.000 | 0.000 | 0.000 | 0.000 | 0.000 | 0.000 | 0.000 | 0.000 | 0.000 | 100.000 | 0.000 | 0.000 | 0.000 |
| BRHIP3013897 | 0.000 | 0.000 | 0.000 | 0.000 | 0.000 | 0.000 | 0.000 | 0.000 | 0.000 | 100.000 | 0.000 | 0.000 | 0.000 |
| BRHIP3015751 | 0.000 | 0.000 | 0.000 | 0.000 | 0.000 | 0.000 | 0.000 | 0.000 | 0.000 | 100.000 | 0.000 | 0.000 | 0.000 |
| BRHIP3016213 | 0.000 | 0.000 | 0.000 | 0.000 | 0.000 | 0.000 | 0.000 | 0.000 | 0.000 | 100.000 | 0.000 | 0.000 | 0.000 |
| BRHIP3018797 | 4.950 | 3.339 | 8.321 | 3.803 | 4.840 | 9.448 | 30.810 | 0.000 | 0.000 | 9.567 | 0.000 | 4.830 | 10.399 |
| BRHIP3020182 | 0.000 | 0.000 | 0.000 | 0.000 | 0.000 | 0.000 | 0.000 | 0.000 | 0.000 | 100.000 | 0.000 | 0.000 | 0.000 |
| BRHIP3024118 | 0.000 | 0.000 | 0.000 | 0.000 | 0.000 | 0.000 | 0.000 | 0.000 | 0.000 | 100.000 | 0.000 | 0.000 | 0.000 |
| BRHIP3024533 | 0.000 | 0.000 | 0.000 | 0.000 | 0.000 | 0.000 | 0.000 | 0.000 | 0.000 | 100.000 | 0.000 | 0.000 | 0.000 |
| BRHIP3024725 | 0.000 | 0.000 | 0.000 | 0.000 | 0.000 | 0.000 | 0.000 | 0.000 | 0.000 | 100.000 | 0.000 | 0.000 | 0.000 |
| BRHIP3025161 | 0.000 | 0.000 | 0.000 | 0.000 | 0.000 | 0.000 | 0.000 | 0.000 | 0.000 | 100.000 | 0.000 | 0.000 | 0.000 |
| BRHIP3025702 | 0.000 | 0.000 | 0.000 | 0.000 | 0.000 | 0.000 | 0.000 | 0.000 | 0.000 | 100.000 | 0.000 | 0.000 | 0.000 |
| BRHIP3026097 | 0.000 | 0.000 | 0.000 | 0.000 | 0.000 | 0.000 | 0.000 | 0.000 | 0.000 | 100.000 | 0.000 | 0.000 | 0.000 |
| BRHIP3027137 | 0.000 | 0.000 | 0.000 | 0.000 | 0.000 | 0.000 | 0.000 | 0.000 | 0.000 | 100.000 | 0.000 | 0.000 | 0.000 |
| BRHIP3027854 | 0.000 | 0.000 | 0.000 | 0.000 | 0.000 | 0.000 | 0.000 | 0.000 | 0.000 | 100.000 | 0.000 | 0.000 | 0.000 |
| BRSSN2000684 | 0.000 | 0.000 | 0.000 | 0.000 | 0.000 | 0.000 | 23.433 | 0.000 | 0.000 | 19.126 | 17.444 | 0.000 | 10.395 |
| BRSSN2003086 | 0.000 | 0.000 | 0.000 | 0.000 | 0.000 | 0.000 | 0.000 | 0.000 | 0.000 | 0.000 | 100.000 | 0.000 | 0.000 |
| BRSSN2004496 | 0.000 | 0.000 | 0.000 | 0.000 | 0.000 | 21.304 | 0.000 | 0.000 | 0.000 | 0.000 | 78.696 | 0.000 | 0.000 |
| BRSSN2004719 | 0.000 | 0.000 | 0.000 | 0.000 | 0.000 | 0.000 | 0.000 | 12.020 | 0.000 | 10.691 | 39.002 | 18.892 | 0.000 |
| BRSSN2006892 | 0.000 | 0.000 | 0.000 | 10.990 | 0.000 | 0.000 | 0.000 | 0.000 | 0.000 | 0.000 | 57.631 | 0.000 | 0.000 |
| BRSSN2008549 | 0.000 | 0.000 | 0.000 | 0.000 | 0.000 | 0.000 | 0.000 | 0.000 | 0.000 | 17.436 | 63.611 | 0.000 | 18.953 |
| BRSSN2008797 | 0.000 | 0.000 | 0.000 | 0.000 | 0.000 | 0.000 | 0.000 | 0.000 | 0.000 | 0.000 | 77.045 | 0.000 | 22.955 |
| BRSSN2011262 | 0.000 | 0.000 | 0.000 | 2.000 | 0.000 | 0.000 | 0.000 | 0.000 | 0.000 | 0.000 | 10.489 | 0.000 | 0.000 |
| BRSSN2011738 | 0.000 | 0.000 | 13.066 | 7.465 | 0.000 | 0.000 | 31.553 | 0.000 | 0.000 | 0.000 | 39.146 | 0.000 | 0.000 |
| BRSSN2013874 | 0.000 | 0.000 | 0.000 | 0.000 | 0.000 | 0.000 | 0.000 | 0.000 | 0.000 | 0.000 | 100.000 | 0.000 | 0.000 |
| BRSSN2014299 | 0.000 | 0.000 | 0.000 | 2.623 | 0.000 | 0.000 | 3.695 | 0.000 | 0.000 | 3.770 | 13.753 | 0.000 | 0.000 |
| BRSSN2014424 | 0.000 | 0.000 | 0.000 | 0.000 | 0.000 | 0.000 | 0.000 | 38.134 | 0.000 | 0.000 | 61.866 | 0.000 | 0.000 |
| BRSSN2014556 | 0.000 | 0.000 | 0.000 | 0.000 | 0.000 | 0.000 | 0.000 | 0.000 | 0.000 | 0.000 | 100.000 | 0.000 | 0.000 |
| BRSSN2018581 | 0.000 | 0.000 | 0.000 | 0.000 | 0.000 | 0.000 | 0.000 | 0.000 | 0.000 | 0.000 | 50.137 | 0.000 | 0.000 |
| BRSSN2018925 | 0.000 | 0.000 | 0.000 | 0.000 | 0.000 | 0.000 | 0.000 | 0.000 | 0.000 | 0.000 | 100.000 | 0.000 | 0.000 |
| BRSTN2000872 | 0.000 | 0.000 | 0.000 | 0.000 | 0.000 | 0.000 | 0.000 | 0.000 | 0.000 | 0.000 | 0.000 | 100.000 | 0.000 |
| BRSTN2001067 | 0.000 | 0.000 | 0.000 | 0.000 | 0.000 | 0.000 | 0.000 | 0.000 | 0.000 | 0.000 | 0.000 | 44.646 | 0.000 |
| BRSTN2001613 | 0.000 | 0.000 | 0.000 | 0.000 | 0.000 | 0.000 | 0.000 | 0.000 | 0.000 | 0.000 | 0.000 | 100.000 | 0.000 |
| BRSTN2002400 | 0.000 | 0.000 | 0.000 | 0.000 | 0.000 | 0.000 | 0.000 | 0.000 | 0.000 | 0.000 | 0.000 | 100.000 | 0.000 |
| BRSTN2003835 | 0.000 | 0.000 | 0.000 | 0.000 | 0.000 | 0.000 | 0.000 | 0.000 | 0.000 | 0.000 | 0.000 | 100.000 | 0.000 |
| BRSTN2004863 | 0.000 | 0.000 | 0.000 | 0.000 | 0.000 | 0.000 | 0.000 | 0.000 | 35.295 | 0.000 | 0.000 | 35.924 | 11.049 |
| BRSTN2004987 | 0.000 | 0.000 | 0.000 | 0.000 | 0.000 | 0.000 | 0.000 | 0.000 | 0.000 | 0.000 | 0.000 | 100.000 | 0.000 |
| BRSTN2005721 | 0.000 | 0.000 | 0.000 | 0.000 | 0.000 | 0.000 | 0.000 | 0.000 | 0.000 | 0.000 | 0.000 | 100.000 | 0.000 |
| BRSTN2006865 | 0.000 | 0.000 | 0.000 | 0.000 | 0.000 | 0.000 | 0.000 | 0.000 | 0.000 | 0.000 | 0.000 | 100.000 | 0.000 |
| BRSTN2007000 | 0.000 | 0.000 | 0.000 | 0.000 | 0.000 | 0.000 | 0.000 | 0.000 | 0.000 | 0.000 | 0.000 | 100.000 | 0.000 |
| BRSTN2007284 | 0.000 | 0.000 | 0.000 | 0.000 | 0.000 | 0.000 | 0.000 | 38.885 | 0.000 | 0.000 | 0.000 | 61.115 | 0.000 |
| BRSTN2008052 | 0.000 | 0.000 | 0.000 | 15.541 | 0.000 | 0.000 | 32.844 | 0.000 | 0.000 | 0.000 | 0.000 | 39.475 | 12.141 |
| BRSTN2008283 | 0.000 | 0.000 | 0.000 | 0.000 | 0.000 | 0.000 | 0.000 | 0.000 | 0.000 | 0.000 | 0.000 | 100.000 | 0.000 |
| BRSTN2008418 | 0.000 | 0.000 | 0.000 | 0.000 | 0.000 | 0.000 | 0.000 | 0.000 | 0.000 | 0.000 | 0.000 | 100.000 | 0.000 |
| BRSTN2008457 | 0.000 | 0.000 | 0.000 | 0.000 | 0.000 | 0.000 | 0.000 | 0.000 | 0.000 | 0.000 | 0.000 | 100.000 | 0.000 |
| BRSTN2010363 | 0.000 | 0.000 | 0.000 | 0.000 | 0.000 | 0.000 | 0.000 | 0.000 | 0.000 | 0.000 | 0.000 | 100.000 | 0.000 |
| BRSTN2010500 | 0.000 | 0.000 | 0.000 | 0.000 | 0.000 | 0.000 | 0.000 | 0.000 | 0.000 | 0.000 | 0.000 | 100.000 | 0.000 |
| BRSTN2010750 | 0.000 | 25.357 | 0.000 | 7.220 | 0.000 | 30.747 | 0.000 | 0.000 | 0.000 | 0.000 | 0.000 | 36.677 | 0.000 |
| BRSTN2012320 | 0.000 | 0.000 | 0.000 | 0.000 | 0.000 | 0.000 | 0.000 | 0.000 | 0.000 | 0.000 | 0.000 | 100.000 | 0.000 |
| BRSTN2012380 | 0.000 | 0.000 | 0.000 | 0.000 | 0.000 | 0.000 | 0.000 | 0.000 | 0.000 | 0.000 | 0.000 | 100.000 | 0.000 |
| BRSTN2015015 | 0.000 | 0.000 | 0.000 | 19.897 | 0.000 | 0.000 | 14.017 | 0.000 | 0.000 | 0.000 | 0.000 | 50.541 | 15.544 |
| BRSTN2016470 | 0.000 | 0.546 | 0.000 | 7.146 | 7.909 | 0.441 | 0.438 | 1.004 | 0.000 | 0.447 | 0.000 | 5.524 | 0.000 |
| BRSTN2016678 | 0.000 | 0.000 | 0.000 | 0.000 | 0.000 | 0.000 | 0.000 | 0.000 | 0.000 | 0.000 | 0.000 | 33.084 | 0.000 |
| BRSTN2017237 | 0.000 | 0.000 | 0.000 | 0.000 | 0.000 | 0.000 | 0.000 | 0.000 | 0.000 | 0.000 | 0.000 | 100.000 | 0.000 |
| BRSTN2017771 | 0.000 | 0.000 | 0.000 | 0.000 | 0.000 | 0.000 | 0.000 | 0.000 | 0.000 | 0.000 | 0.000 | 100.000 | 0.000 |
| BRSTN2018083 | 0.000 | 0.000 | 0.000 | 0.000 | 0.000 | 0.000 | 0.000 | 0.000 | 0.000 | 22.055 | 0.000 | 77.945 | 0.000 |
| BRSTN2019129 | 0.000 | 0.000 | 0.000 | 0.000 | 0.000 | 0.000 | 0.000 | 0.000 | 0.000 | 0.000 | 0.000 | 100.000 | 0.000 |
| BRTHA1000311 | 0.000 | 0.000 | 7.331 | 10.472 | 0.000 | 14.866 | 11.803 | 6.770 | 0.000 | 6.021 | 10.982 | 0.000 | 9.817 |
| BRTHA2000855 | 0.000 | 0.000 | 0.000 | 0.000 | 0.000 | 0.000 | 0.000 | 0.000 | 0.000 | 0.000 | 0.000 | 0.000 | 100.000 |
| BRTHA2001462 | 0.000 | 0.000 | 0.000 | 0.000 | 0.000 | 0.000 | 0.000 | 0.000 | 0.000 | 0.000 | 0.000 | 0.000 | 100.000 |
| BRTHA2002115 | 0.000 | 0.000 | 0.000 | 0.000 | 0.000 | 0.000 | 0.000 | 0.000 | 0.000 | 0.000 | 0.000 | 0.000 | 100.000 |
| BRTHA2002281 | 0.000 | 0.000 | 0.000 | 0.000 | 0.000 | 0.000 | 0.000 | 0.000 | 0.000 | 0.000 | 0.000 | 0.000 | 100.000 |
| BRTHA2002376 | 0.000 | 0.000 | 0.000 | 0.000 | 0.000 | 0.000 | 0.000 | 0.000 | 0.000 | 0.000 | 0.000 | 0.000 | 100.000 |
| BRTHA2002442 | 0.000 | 0.000 | 0.000 | 0.000 | 0.000 | 0.000 | 0.000 | 0.000 | 0.000 | 31.507 | 0.000 | 0.000 | 68.493 |
| BRTHA2002493 | 0.000 | 0.000 | 0.000 | 0.000 | 0.000 | 0.000 | 0.000 | 0.000 | 0.000 | 0.000 | 0.000 | 0.000 | 100.000 |
| BRTHA2002608 | 0.000 | 0.000 | 0.000 | 0.000 | 0.000 | 0.000 | 0.000 | 0.000 | 0.000 | 0.000 | 0.000 | 0.000 | 100.000 |
| BRTHA2002808 | 0.000 | 0.000 | 0.000 | 0.000 | 0.000 | 0.000 | 0.000 | 0.000 | 0.000 | 0.000 | 0.000 | 0.000 | 12.857 |
| BRTHA2003030 | 0.000 | 0.000 | 0.000 | 0.000 | 0.000 | 0.000 | 0.000 | 0.000 | 0.000 | 0.000 | 0.000 | 0.000 | 100.000 |
| BRTHA2003110 | 0.000 | 0.000 | 0.000 | 0.000 | 0.000 | 0.000 | 0.000 | 0.000 | 0.000 | 0.000 | 0.000 | 0.000 | 100.000 |
| BRTHA2003116 | 0.000 | 0.000 | 0.000 | 0.000 | 0.000 | 0.000 | 0.000 | 0.000 | 0.000 | 0.000 | 0.000 | 0.000 | 100.000 |
| BRTHA2003461 | 4.055 | 0.000 | 0.000 | 0.000 | 0.000 | 0.000 | 0.000 | 0.000 | 7.775 | 0.000 | 8.169 | 0.000 | 2.434 |
| BRTHA2004821 | 0.000 | 0.000 | 0.000 | 0.000 | 0.000 | 0.000 | 0.000 | 0.000 | 0.000 | 0.000 | 0.000 | 0.000 | 100.000 |
| BRTHA2004978 | 0.000 | 0.000 | 0.000 | 0.000 | 0.000 | 0.000 | 0.000 | 0.000 | 0.000 | 0.000 | 0.000 | 0.000 | 100.000 |
| BRTHA2005579 | 0.000 | 0.000 | 0.000 | 0.000 | 0.000 | 0.000 | 0.000 | 0.000 | 0.000 | 0.000 | 0.000 | 0.000 | 100.000 |
| BRTHA2005956 | 0.000 | 0.000 | 0.000 | 0.000 | 0.000 | 0.000 | 0.000 | 0.000 | 0.000 | 0.000 | 0.000 | 0.000 | 100.000 |
| BRTHA2006075 | 0.000 | 0.000 | 0.000 | 0.000 | 0.000 | 0.000 | 0.000 | 0.000 | 0.000 | 0.000 | 0.000 | 0.000 | 100.000 |
| BRTHA2006146 | 0.000 | 0.000 | 0.000 | 0.000 | 0.000 | 0.000 | 0.000 | 0.000 | 0.000 | 0.000 | 0.000 | 0.000 | 100.000 |
| BRTHA2006194 | 0.000 | 0.000 | 0.000 | 0.000 | 0.000 | 0.000 | 0.000 | 0.000 | 0.000 | 0.000 | 0.000 | 0.000 | 100.000 |
| BRTHA2007122 | 0.000 | 0.000 | 0.000 | 0.000 | 0.000 | 0.000 | 0.000 | 0.000 | 0.000 | 0.000 | 0.000 | 0.000 | 100.000 |
| BRTHA2007422 | 0.000 | 0.000 | 0.000 | 0.000 | 0.000 | 0.000 | 0.000 | 0.000 | 0.000 | 0.000 | 0.000 | 0.000 | 100.000 |
| BRTHA2007603 | 0.000 | 0.000 | 0.000 | 0.000 | 0.000 | 0.000 | 0.000 | 0.000 | 0.000 | 0.000 | 0.000 | 0.000 | 100.000 |
| BRTHA2008316 | 0.000 | 0.000 | 0.000 | 0.000 | 0.000 | 0.000 | 0.000 | 0.000 | 0.000 | 0.000 | 0.000 | 0.000 | 100.000 |
| BRTHA2008335 | 0.000 | 0.000 | 0.000 | 0.000 | 0.000 | 0.000 | 16.281 | 0.000 | 0.000 | 8.305 | 0.000 | 0.000 | 9.028 |
| BRTHA2008527 | 0.000 | 0.000 | 0.000 | 0.000 | 0.000 | 0.000 | 0.000 | 0.000 | 0.000 | 0.000 | 0.000 | 0.000 | 100.000 |
| BRTHA2008535 | 0.000 | 0.000 | 0.000 | 0.000 | 0.000 | 0.000 | 0.000 | 0.000 | 0.000 | 0.000 | 0.000 | 0.000 | 100.000 |
| BRTHA2008955 | 0.000 | 0.000 | 0.000 | 39.025 | 0.000 | 0.000 | 0.000 | 0.000 | 0.000 | 0.000 | 0.000 | 0.000 | 60.975 |
| BRTHA2009311 | 0.000 | 0.000 | 0.000 | 0.000 | 0.000 | 0.000 | 0.000 | 0.000 | 0.000 | 0.000 | 0.000 | 0.000 | 100.000 |
| BRTHA2009846 | 0.000 | 0.000 | 0.000 | 0.000 | 0.000 | 0.000 | 0.000 | 0.000 | 0.000 | 0.000 | 0.000 | 0.000 | 100.000 |
| BRTHA2009972 | 0.000 | 0.000 | 0.000 | 0.000 | 0.000 | 0.000 | 0.000 | 0.000 | 0.000 | 0.000 | 0.000 | 0.000 | 100.000 |
| BRTHA2010073 | 0.000 | 0.000 | 0.000 | 0.000 | 0.000 | 0.000 | 0.000 | 0.000 | 0.000 | 0.000 | 0.000 | 0.000 | 100.000 |
| BRTHA2010608 | 0.000 | 0.000 | 0.000 | 0.000 | 0.000 | 0.000 | 0.000 | 0.000 | 0.000 | 0.000 | 0.000 | 0.000 | 48.181 |
| BRTHA2010884 | 0.000 | 0.000 | 0.000 | 0.000 | 0.000 | 0.000 | 0.000 | 0.000 | 0.000 | 0.000 | 0.000 | 0.000 | 100.000 |
| BRTHA2010907 | 0.000 | 0.000 | 0.000 | 0.000 | 0.000 | 0.000 | 0.000 | 0.000 | 0.000 | 0.000 | 0.000 | 0.000 | 100.000 |
| BRTHA2011194 | 0.000 | 0.000 | 0.000 | 39.025 | 0.000 | 0.000 | 0.000 | 0.000 | 0.000 | 0.000 | 0.000 | 0.000 | 60.975 |
| BRTHA2011351 | 0.000 | 0.000 | 0.000 | 0.000 | 0.000 | 0.000 | 0.000 | 0.000 | 0.000 | 0.000 | 0.000 | 0.000 | 100.000 |
| BRTHA2011500 | 0.000 | 0.000 | 0.000 | 0.000 | 0.000 | 0.000 | 0.000 | 0.000 | 0.000 | 0.000 | 0.000 | 0.000 | 100.000 |
| BRTHA2011641 | 0.000 | 0.000 | 0.000 | 0.000 | 0.000 | 47.605 | 0.000 | 0.000 | 0.000 | 0.000 | 0.000 | 0.000 | 52.395 |
| BRTHA2012392 | 0.000 | 0.000 | 0.000 | 0.000 | 0.000 | 0.000 | 0.000 | 0.000 | 0.000 | 0.000 | 0.000 | 0.000 | 38.390 |
| BRTHA2012562 | 0.000 | 0.000 | 0.000 | 0.000 | 0.000 | 0.000 | 0.000 | 0.000 | 0.000 | 0.000 | 0.000 | 0.000 | 100.000 |
| BRTHA2012980 | 0.000 | 0.000 | 0.000 | 0.000 | 0.000 | 0.000 | 0.000 | 0.000 | 0.000 | 0.000 | 0.000 | 0.000 | 13.890 |
| BRTHA2013262 | 0.000 | 0.000 | 0.000 | 0.000 | 0.000 | 0.000 | 0.000 | 0.000 | 0.000 | 0.000 | 0.000 | 0.000 | 100.000 |
| BRTHA2013460 | 0.000 | 0.000 | 0.000 | 0.000 | 0.000 | 0.000 | 0.000 | 0.000 | 0.000 | 0.000 | 0.000 | 0.000 | 100.000 |
| BRTHA2013707 | 0.000 | 0.000 | 0.000 | 0.000 | 0.000 | 0.000 | 0.000 | 0.000 | 0.000 | 0.000 | 0.000 | 0.000 | 100.000 |
| BRTHA2014792 | 0.000 | 0.000 | 0.000 | 0.000 | 0.000 | 0.000 | 0.000 | 0.000 | 0.000 | 0.000 | 0.000 | 0.000 | 100.000 |
| BRTHA2014828 | 0.000 | 0.000 | 0.000 | 0.000 | 0.000 | 0.000 | 0.000 | 0.000 | 0.000 | 0.000 | 0.000 | 0.000 | 100.000 |
| BRTHA2015406 | 0.000 | 0.000 | 0.000 | 0.000 | 0.000 | 0.000 | 0.000 | 0.000 | 0.000 | 0.000 | 0.000 | 0.000 | 100.000 |
| BRTHA2015478 | 0.000 | 0.000 | 0.000 | 0.000 | 0.000 | 0.000 | 0.000 | 0.000 | 0.000 | 0.000 | 0.000 | 0.000 | 100.000 |
| BRTHA2015696 | 0.000 | 0.000 | 0.000 | 0.000 | 0.000 | 0.000 | 0.000 | 0.000 | 0.000 | 0.000 | 0.000 | 0.000 | 100.000 |
| BRTHA2015878 | 0.000 | 0.000 | 0.000 | 0.000 | 0.000 | 0.000 | 0.000 | 0.000 | 0.000 | 0.000 | 0.000 | 0.000 | 100.000 |
| BRTHA2016215 | 0.000 | 0.000 | 0.000 | 0.000 | 0.000 | 0.000 | 0.000 | 0.000 | 0.000 | 0.000 | 0.000 | 0.000 | 38.930 |
| BRTHA2016496 | 0.000 | 0.000 | 0.000 | 0.000 | 0.000 | 0.000 | 0.000 | 0.000 | 0.000 | 0.000 | 0.000 | 0.000 | 100.000 |
| BRTHA2016543 | 0.000 | 0.000 | 0.000 | 0.000 | 0.000 | 0.000 | 0.000 | 0.000 | 0.000 | 0.000 | 0.000 | 0.000 | 100.000 |
| BRTHA2017353 | 0.000 | 0.000 | 0.000 | 0.000 | 0.000 | 0.000 | 0.000 | 0.000 | 0.000 | 0.000 | 0.000 | 0.000 | 100.000 |
| BRTHA2017985 | 0.000 | 0.000 | 0.000 | 0.000 | 0.000 | 0.000 | 0.000 | 0.000 | 0.000 | 0.000 | 0.000 | 0.000 | 49.749 |
| BRTHA2018165 | 0.000 | 0.000 | 0.000 | 0.000 | 0.000 | 0.000 | 0.000 | 0.000 | 0.000 | 0.000 | 0.000 | 0.000 | 100.000 |
| BRTHA2018344 | 0.000 | 0.000 | 0.000 | 0.000 | 0.000 | 0.000 | 0.000 | 0.000 | 0.000 | 0.000 | 0.000 | 0.000 | 100.000 |
| BRTHA2018591 | 0.000 | 0.000 | 0.000 | 0.000 | 0.000 | 0.000 | 0.000 | 0.000 | 0.000 | 0.000 | 0.000 | 0.000 | 100.000 |
| BRTHA2018624 | 0.000 | 0.000 | 0.000 | 0.000 | 0.000 | 0.000 | 0.000 | 0.000 | 0.000 | 0.000 | 0.000 | 0.000 | 100.000 |
| BRTHA2018707 | 0.000 | 0.000 | 0.000 | 0.000 | 0.000 | 0.000 | 0.000 | 0.000 | 0.000 | 0.000 | 0.000 | 0.000 | 57.080 |
| BRTHA2019014 | 0.000 | 0.000 | 0.000 | 0.000 | 0.000 | 0.000 | 0.000 | 0.000 | 0.000 | 0.000 | 0.000 | 0.000 | 100.000 |
| BRTHA2019022 | 0.000 | 0.000 | 0.000 | 0.000 | 0.000 | 0.000 | 0.000 | 0.000 | 0.000 | 0.000 | 0.000 | 0.000 | 100.000 |
| BRTHA2019048 | 0.000 | 0.000 | 0.000 | 0.000 | 0.000 | 0.000 | 0.000 | 0.000 | 0.000 | 0.000 | 0.000 | 0.000 | 100.000 |
| BRTHA3000273 | 0.000 | 0.000 | 0.000 | 0.000 | 0.000 | 0.000 | 0.000 | 0.000 | 0.000 | 0.000 | 0.000 | 0.000 | 57.080 |
| BRTHA3000297 | 0.000 | 0.000 | 0.000 | 0.000 | 0.000 | 0.000 | 0.000 | 0.000 | 0.000 | 47.917 | 0.000 | 0.000 | 52.083 |
| BRTHA3000633 | 0.000 | 0.000 | 0.000 | 0.000 | 0.000 | 0.000 | 0.000 | 0.000 | 0.000 | 0.000 | 0.000 | 0.000 | 37.898 |
| BRTHA3001721 | 0.000 | 0.000 | 14.633 | 8.360 | 0.000 | 23.737 | 0.000 | 0.000 | 0.000 | 12.017 | 0.000 | 0.000 | 13.062 |
| BRTHA3002401 | 0.000 | 0.000 | 0.000 | 0.000 | 0.000 | 0.000 | 0.000 | 0.000 | 0.000 | 0.000 | 0.000 | 0.000 | 100.000 |
| BRTHA3002427 | 0.000 | 0.000 | 3.551 | 2.029 | 0.000 | 5.761 | 8.577 | 0.000 | 10.128 | 2.917 | 10.641 | 10.308 | 15.852 |
| BRTHA3002933 | 0.000 | 0.000 | 0.000 | 0.000 | 0.000 | 0.000 | 0.000 | 0.000 | 0.000 | 0.000 | 0.000 | 0.000 | 100.000 |
| BRTHA3003074 | 0.000 | 0.000 | 0.000 | 0.000 | 0.000 | 0.000 | 0.000 | 0.000 | 0.000 | 0.000 | 0.000 | 0.000 | 37.459 |
| BRTHA3003343 | 0.000 | 0.000 | 0.000 | 0.000 | 0.000 | 0.000 | 0.000 | 0.000 | 0.000 | 0.000 | 0.000 | 0.000 | 100.000 |
| BRTHA3003449 | 0.000 | 0.000 | 0.000 | 0.000 | 0.000 | 0.000 | 0.000 | 0.000 | 0.000 | 0.000 | 0.000 | 0.000 | 100.000 |
| BRTHA3003474 | 0.000 | 0.000 | 0.000 | 0.000 | 0.000 | 0.000 | 0.000 | 0.000 | 0.000 | 0.000 | 0.000 | 0.000 | 100.000 |
| BRTHA3003490 | 0.000 | 0.000 | 8.067 | 2.304 | 0.000 | 0.000 | 1.623 | 3.724 | 0.000 | 0.000 | 0.000 | 0.000 | 1.800 |
| BRTHA3004475 | 0.000 | 0.000 | 0.000 | 0.000 | 0.000 | 47.605 | 0.000 | 0.000 | 0.000 | 0.000 | 0.000 | 0.000 | 52.395 |
| 8RTHA3005046 | 0.000 | 0.000 | 0.000 | 0.000 | 0.000 | 0.000 | 0.000 | 0.000 | 0.000 | 47.917 | 0.000 | 0.000 | 52.083 |
| BRTHA3006856 | 0.000 | 0.000 | 0.000 | 0.000 | 0.000 | 0.000 | 0.000 | 0.000 | 0.000 | 0.000 | 0.000 | 0.000 | 100.000 |
| BRTHA3007113 | 0.000 | 0.000 | 0.000 | 0.000 | 0.000 | 0.000 | 0.000 | 0.000 | 0.000 | 0.000 | 0.000 | 0.000 | 100.000 |
| BRTHA3007148 | 0.000 | 0.000 | 0.000 | 0.000 | 0.000 | 0.000 | 0.000 | 0.000 | 0.000 | 0.000 | 0.000 | 0.000 | 100.000 |
| BRTHA3007319 | 0.000 | 0.000 | 0.000 | 0.000 | 0.000 | 0.000 | 0.000 | 0.000 | 0.000 | 0.000 | 0.000 | 0.000 | 100.000 |
| BRTHA3007769 | 0.000 | 0.000 | 0.000 | 0.000 | 0.000 | 0.000 | 0.000 | 0.000 | 0.000 | 0.000 | 0.000 | 0.000 | 100.000 |
| BRTHA3008143 | 0.000 | 0.000 | 0.000 | 0.000 | 0.000 | 0.000 | 0.000 | 0.000 | 0.000 | 0.000 | 0.000 | 0.000 | 100.000 |
| BRTHA3008310 | 0.000 | 0.000 | 0.000 | 0.000 | 0.000 | 0.000 | 0.000 | 0.000 | 0.000 | 0.000 | 0.000 | 0.000 | 100.000 |
| BRTHA3008386 | 0.000 | 0.000 | 0.000 | 0.000 | 0.000 | 0.000 | 0.000 | 0.000 | 0.000 | 0.000 | 0.000 | 0.000 | 100.000 |
| BRTHA3008520 | 0.000 | 0.000 | 0.000 | 0.000 | 0.000 | 0.000 | 47.417 | 0.000 | 0.000 | 0.000 | 0.000 | 0.000 | 52.583 |
| BRTHA3008778 | 0.000 | 0.000 | 30.863 | 0.000 | 0.000 | 0.000 | 0.000 | 0.000 | 0.000 | 25.346 | 0.000 | 0.000 | 27.551 |
| BRTHA3009037 | 0.000 | 0.000 | 0.000 | 0.000 | 0.000 | 0.000 | 0.000 | 0.000 | 0.000 | 0.000 | 0.000 | 0.000 | 100.000 |
| BRTHA3009090 | 0.000 | 29.341 | 0.000 | 8.354 | 0.000 | 11.859 | 0.000 | 0.000 | 0.000 | 12.008 | 0.000 | 0.000 | 13.052 |
| BRTHA3009291 | 0.000 | 0.000 | 0.000 | 0.000 | 0.000 | 0.000 | 0.000 | 0.000 | 0.000 | 0.000 | 0.000 | 0.000 | 100.000 |
| BRTHA3010366 | 0.000 | 0.000 | 0.000 | 0.000 | 0.000 | 0.000 | 0.000 | 0.000 | 0.000 | 0.000 | 0.000 | 0.000 | 100.000 |
| BRTHA3013884 | 0.000 | 0.000 | 0.000 | 0.000 | 0.000 | 0.000 | 0.000 | 0.000 | 0.000 | 0.000 | 0.000 | 0.000 | 100.000 |
| BRTHA3015815 | 0.000 | 0.000 | 0.000 | 0.000 | 0.000 | 0.000 | 0.000 | 0.000 | 0.000 | 0.000 | 0.000 | 0.000 | 100.000 |
| BRTHA3015910 | 0.000 | 0.000 | 0.000 | 0.000 | 0.000 | 0.000 | 0.000 | 0.000 | 0.000 | 15.221 | 0.000 | 0.000 | 33.089 |
| BRTHA3016845 | 0.000 | 0.000 | 0.000 | 0.000 | 0.000 | 0.000 | 0.000 | 0.000 | 0.000 | 0.000 | 0.000 | 0.000 | 100.000 |
| BRTHA3016917 | 0.000 | 0.000 | 0.000 | 0.000 | 0.000 | 0.000 | 0.000 | 0.000 | 0.000 | 0.000 | 0.000 | 0.000 | 100.000 |
| BRTHA3017047 | 0.000 | 0.000 | 0.000 | 0.000 | 0.000 | 0.000 | 0.000 | 0.000 | 0.000 | 0.000 | 0.000 | 0.000 | 100.000 |
| BRTHA3017589 | 0.000 | 0.000 | 0.000 | 0.000 | 0.000 | 0.000 | 0.000 | 0.000 | 0.000 | 0.000 | 0.000 | 0.000 | 100.000 |
| BRTHA3017848 | 0.000 | 0.000 | 0.000 | 0.000 | 0.000 | 0.000 | 47.417 | 0.000 | 0.000 | 0.000 | 0.000 | 0.000 | 52.583 |
| BRTHA3018514 | 0.000 | 0.000 | 0.000 | 0.000 | 0.000 | 0.000 | 0.000 | 0.000 | 0.000 | 0.000 | 0.000 | 0.000 | 100.000 |
| BRTHA3018617 | 0.000 | 0.000 | 0.000 | 0.000 | 0.000 | 0.000 | 0.000 | 0.000 | 0.000 | 0.000 | 0.000 | 0.000 | 100.000 |
| BRTHA3018656 | 0.000 | 0.000 | 0.000 | 0.000 | 0.000 | 0.000 | 0.000 | 0.000 | 0.000 | 0.000 | 0.000 | 0.000 | 100.000 |
| BRTHA3019105 | 0.000 | 0.000 | 0.000 | 0.000 | 0.000 | 0.000 | 0.000 | 0.000 | 0.000 | 0.000 | 0.000 | 0.000 | 100.000 |
| COLON2001721 | 0.000 | 0.000 | 0.000 | 19.633 | 0.000 | 11.149 | 11.065 | 0.000 | 0.000 | 5.644 | 0.000 | 0.000 | 0.000 |
| CTONG1000087 | 1.932 | 2.607 | 1.299 | 0.000 | 0.000 | 3.161 | 0.000 | 2.399 | 0.000 | 1.067 | 0.000 | 0.000 | 3.479 |
| CTONG1000088 | 0.000 | 0.000 | 0.000 | 0.000 | 0.000 | 0.000 | 0.000 | 0.000 | 0.000 | 2.981 | 0.000 | 0.000 | 0.000 |
| CTONG1000467 | 0.000 | 0.000 | 0.000 | 0.000 | 0.000 | 0.000 | 0.000 | 0.000 | 30.440 | 8.766 | 0.000 | 0.000 | 0.000 |
| CTONG2000042 | 0.000 | 0.000 | 0.000 | 0.000 | 0.000 | 0.000 | 0.000 | 0.000 | 0.000 | 3.574 | 0.000 | 0.000 | 0.000 |
| CTONG2008233 | 1.269 | 1.712 | 2.559 | 3.412 | 0.000 | 0.692 | 0.000 | 1.575 | 2.433 | 0.701 | 0.000 | 0.000 | 3.046 |
| CTONG2009423 | 0.000 | 0.000 | 0.000 | 0.000 | 0.000 | 0.000 | 0.000 | 0.000 | 0.000 | 35.391 | 0.000 | 0.000 | 0.000 |
| CTONG2017500 | 0.000 | 0.000 | 3.291 | 0.000 | 0.000 | 2.669 | 2.649 | 0.000 | 0.000 | 0.000 | 0.000 | 0.000 | 2.938 |
| CTONG2019248 | 27.950 | 0.000 | 0.000 | 0.000 | 0.000 | 0.000 | 0.000 | 0.000 | 0.000 | 0.000 | 0.000 | 0.000 | 0.000 |
| CTONG2019788 | 0.000 | 0.000 | 3.798 | 0.000 | 0.000 | 0.000 | 0.000 | 0.000 | 0.000 | 3.120 | 0.000 | 0.000 | 3.391 |
| CTONG2020522 | 42.337 | 0.000 | 0.000 | 0.000 | 0.000 | 0.000 | 0.000 | 0.000 | 0.000 | 0.000 | 0.000 | 0.000 | 0.000 |
| CTONG2023021 | 0.000 | 0.000 | 0.000 | 0.000 | 0.000 | 0.000 | 0.000 | 0.000 | 0.000 | 0.000 | 0.000 | 0.000 | 37.320 |
| CTONG2028124 | 1.857 | 2.506 | 1.249 | 2.854 | 0.000 | 0.506 | 0.503 | 2.306 | 1.780 | 1.026 | 0.000 | 0.000 | 1.672 |
| CTONG3000657 | 0.000 | 0.000 | 0.000 | 5.508 | 0.000 | 0.000 | 3.880 | 8.901 | 0.000 | 11.875 | 0.000 | 0.000 | 8.605 |
| CTONG3001123 | 0.000 | 0.000 | 0.000 | 0.000 | 0.000 | 0.000 | 7.847 | 0.000 | 0.000 | 0.000 | 0.000 | 0.000 | 0.000 |
| CTONG3001370 | 0.000 | 0.000 | 0.000 | 0.000 | 0.000 | 0.000 | 0.000 | 0.000 | 0.000 | 3.574 | 0.000 | 0.000 | 0.000 |
| CTONG3002412 | 0.000 | 0.000 | 0.000 | 0.000 | 0.000 | 0.000 | 0.000 | 0.000 | 0.000 | 10.918 | 0.000 | 0.000 | 0.000 |
| CTONG3004072 | 0.000 | 0.000 | 0.000 | 0.000 | 0.000 | 0.000 | 0.000 | 0.000 | 0.000 | 19.915 | 0.000 | 0.000 | 0.000 |
| CTONG3005813 | 0.000 | 0.000 | 0.000 | 27.592 | 0.000 | 0.000 | 0.000 | 0.000 | 0.000 | 0.000 | 0.000 | 0.000 | 0.000 |
| CTONG3008894 | 2.686 | 0.000 | 12.642 | 1.032 | 0.000 | 4.395 | 0.000 | 0.000 | 0.000 | 2.966 | 0.000 | 0.000 | 2.418 |
| CTONG3009028 | 0.000 | 0.000 | 0.000 | 0.000 | 0.000 | 0.000 | 0.000 | 0.000 | 0.000 | 0.000 | 0.000 | 0.000 | 1.785 |
| CTONG3009239 | 8.002 | 0.000 | 0.000 | 0.000 | 0.000 | 4.364 | 0.000 | 0.000 | 5.115 | 2.946 | 0.000 | 0.000 | 1.601 |
| CTONG3009328 | 0.000 | 0.000 | 0.000 | 0.000 | 43.334 | 0.000 | 11.993 | 0.000 | 0.000 | 0.000 | 0.000 | 0.000 | 0.000 |
| DFNES2000146 | 20.566 | 0.000 | 13.829 | 0.000 | 0.000 | 0.000 | 0.000 | 0.000 | 0.000 | 0.000 | 0.000 | 0.000 | 0.000 |
| DFNES2011239 | 13.804 | 0.000 | 0.000 | 0.000 | 0.000 | 0.000 | 0.000 | 0.000 | 0.000 | 0.000 | 0.000 | 0.000 | 0.000 |
| DFNES2011499 | 0.000 | 0.000 | 0.000 | 4.403 | 0.000 | 0.000 | 0.000 | 0.000 | 0.000 | 6.330 | 0.000 | 0.000 | 0.000 |
| FCBBF1000297 | 100.000 | 0.000 | 0.000 | 0.000 | 0.000 | 0.000 | 0.000 | 0.000 | 0.000 | 0.000 | 0.000 | 0.000 | 0.000 |
| FCBBF2001183 | 6.110 | 4.122 | 8.217 | 5.869 | 5.975 | 9.998 | 16.537 | 3.794 | 0.000 | 11.810 | 6.155 | 0.000 | 9.169 |
| FCBBF3001977 | 13.761 | 0.000 | 9.253 | 10.573 | 0.000 | 15.010 | 7.448 | 0.000 | 0.000 | 7.599 | 0.000 | 0.000 | 0.000 |
| FCBBF3002163 | 100.000 | 0.000 | 0.000 | 0.000 | 0.000 | 0.000 | 0.000 | 0.000 | 0.000 | 0.000 | 0.000 | 0.000 | 0.000 |
| FCBBF3003435 | 33.081 | 0.000 | 0.000 | 0.000 | 0.000 | 0.000 | 0.000 | 0.000 | 0.000 | 0.000 | 0.000 | 0.000 | 0.000 |
| FCBBF3004502 | 100.000 | 0.000 | 0.000 | 0.000 | 0.000 | 0.000 | 0.000 | 0.000 | 0.000 | 0.000 | 0.000 | 0.000 | 0.000 |
| FCBBF3004847 | 60.769 | 0.000 | 0.000 | 0.000 | 0.000 | 0.000 | 0.000 | 0.000 | 0.000 | 0.000 | 0.000 | 0.000 | 0.000 |
| FCBBF3006171 | 100.000 | 0.000 | 0.000 | 0.000 | 0.000 | 0.000 | 0.000 | 0.000 | 0.000 | 0.000 | 0.000 | 0.000 | 0.000 |
| FCBBF3007242 | 100.000 | 0.000 | 0.000 | 0.000 | 0.000 | 0.000 | 0.000 | 0.000 | 0.000 | 0.000 | 0.000 | 0.000 | 0.000 |
| FCBBF3007540 | 100.000 | 0.000 | 0.000 | 0.000 | 0.000 | 0.000 | 0.000 | 0.000 | 0.000 | 0.000 | 0.000 | 0.000 | 0.000 |
| FCBBF3008944 | 100.000 | 0.000 | 0.000 | 0.000 | 0.000 | 0.000 | 0.000 | 0.000 | 0.000 | 0.000 | 0.000 | 0.000 | 0.000 |
| FCBBF3009888 | 100.000 | 0.000 | 0.000 | 0.000 | 0.000 | 0.000 | 0.000 | 0.000 | 0.000 | 0.000 | 0.000 | 0.000 | 0.000 |
| FCBBF3012170 | 100.000 | 0.000 | 0.000 | 0.000 | 0.000 | 0.000 | 0.000 | 0.000 | 0.000 | 0.000 | 0.000 | 0.000 | 0.000 |
| FCBBF3012288 | 73.865 | 0.000 | 0.000 | 0.000 | 0.000 | 0.000 | 0.000 | 0.000 | 0.000 | 0.000 | 0.000 | 0.000 | 0.000 |
| FCBBF3013307 | 100.000 | 0.000 | 0.000 | 0.000 | 0.000 | 0.000 | 0.000 | 0.000 | 0.000 | 0.000 | 0.000 | 0.000 | 0.000 |
| FCBBF3013846 | 100.000 | 0.000 | 0.000 | 0.000 | 0.000 | 0.000 | 0.000 | 0.000 | 0.000 | 0.000 | 0.000 | 0.000 | 0.000 |
| FCBBF3021576 | 15.878 | 0.000 | 10.677 | 0.000 | 0.000 | 0.000 | 0.000 | 0.000 | 0.000 | 0.000 | 0.000 | 0.000 | 19.062 |
| FCBBF3021940 | 46.137 | 0.000 | 0.000 | 0.000 | 0.000 | 0.000 | 0.000 | 0.000 | 0.000 | 0.000 | 0.000 | 0.000 | 0.000 |
| FCBBF3023443 | 100.000 | 0.000 | 0.000 | 0.000 | 0.000 | 0.000 | 0.000 | 0.000 | 0.000 | 0.000 | 0.000 | 0.000 | 0.000 |
| FCBBF3023895 | 100.000 | 0.000 | 0.000 | 0.000 | 0.000 | 0.000 | 0.000 | 0.000 | 0.000 | 0.000 | 0.000 | 0.000 | 0.000 |
| FCBBF3025730 | 36.577 | 0.000 | 24.596 | 0.000 | 0.000 | 0.000 | 0.000 | 0.000 | 0.000 | 0.000 | 0.000 | 0.000 | 0.000 |
| FCBBF3027717 | 100.000 | 0.000 | 0.000 | 0.000 | 0.000 | 0.000 | 0.000 | 0.000 | 0.000 | 0.000 | 0.000 | 0.000 | 0.000 |
| FCBBF4000076 | 100.000 | 0.000 | 0.000 | 0.000 | 0.000 | 0.000 | 0.000 | 0.000 | 0.000 | 0.000 | 0.000 | 0.000 | 0.000 |
| FEBRA1000030 | 0.000 | 40.753 | 0.000 | 0.000 | 0.000 | 0.000 | 0.000 | 0.000 | 0.000 | 0.000 | 0.000 | 0.000 | 0.000 |
| FEBRA2000253 | 0.000 | 36.491 | 36.369 | 0.000 | 0.000 | 0.000 | 0.000 | 0.000 | 0.000 | 14.934 | 0.000 | 0.000 | 0.000 |
| FEBRA2006396 | 29.698 | 20.037 | 29.955 | 5.705 | 0.000 | 0.000 | 0.000 | 0.000 | 0.000 | 0.000 | 0.000 | 0.000 | 0.000 |
| FEBRA2007544 | 4.523 | 12.206 | 9.124 | 41.703 | 0.000 | 0.000 | 14.689 | 0.000 | 0.000 | 4.995 | 0.000 | 0.000 | 2.715 |
| FEBRA2007708 | 0.000 | 21.306 | 0.000 | 6.066 | 0.000 | 0.000 | 0.000 | 0.000 | 0.000 | 0.000 | 0.000 | 0.000 | 0.000 |
| FEBRA2007793 | 0.000 | 50.083 | 49.917 | 0.000 | 0.000 | 0.000 | 0.000 | 0.000 | 0.000 | 0.000 | 0.000 | 0.000 | 0.000 |
| FEBRA2007801 | 4.561 | 6.154 | 0.000 | 1.752 | 0.000 | 0.000 | 4.937 | 0.000 | 8.745 | 7.556 | 0.000 | 0.000 | 5.475 |
| FEBRA2008287 | 0.000 | 7.348 | 3.662 | 4.184 | 0.000 | 2.970 | 0.000 | 0.000 | 0.000 | 3.007 | 0.000 | 0.000 | 6.537 |
| FEBRA2008311 | 0.000 | 100.000 | 0.000 | 0.000 | 0.000 | 0.000 | 0.000 | 0.000 | 0.000 | 0.000 | 0.000 | 0.000 | 0.000 |
| FEBRA2008360 | 37.775 | 25.486 | 25.401 | 0.000 | 0.000 | 0.000 | 0.000 | 0.000 | 0.000 | 0.000 | 0.000 | 0.000 | 11.338 |
| FEBRA2008468 | 0.000 | 100.000 | 0.000 | 0.000 | 0.000 | 0.000 | 0.000 | 0.000 | 0.000 | 0.000 | 0.000 | 0.000 | 0.000 |
| FEBRA2010719 | 0.000 | 43.257 | 21.556 | 0.000 | 0.000 | 17.484 | 0.000 | 0.000 | 0.000 | 17.703 | 0.000 | 0.000 | 0.000 |
| FEBRA2014213 | 0.000 | 9.817 | 0.000 | 0.000 | 0.000 | 0.000 | 0.000 | 0.000 | 0.000 | 0.000 | 0.000 | 0.000 | 0.000 |
| FEBRA2015588 | 0.000 | 100.000 | 0.000 | 0.000 | 0.000 | 0.000 | 0.000 | 0.000 | 0.000 | 0.000 | 0.000 | 0.000 | 0.000 |
| FEBRA2020484 | 13.507 | 63.788 | 22.706 | 0.000 | 0.000 | 0.000 | 0.000 | 0.000 | 0.000 | 0.000 | 0.000 | 0.000 | 0.000 |
| FEBRA2020582 | 42.565 | 57.435 | 0.000 | 0.000 | 0.000 | 0.000 | 0.000 | 0.000 | 0.000 | 0.000 | 0.000 | 0.000 | 0.000 |
| FEBRA2020668 | 0.000 | 100.000 | 0.000 | 0.000 | 0.000 | 0.000 | 0.000 | 0.000 | 0.000 | 0.000 | 0.000 | 0.000 | 0.000 |
| FEBRA2020886 | 0.000 | 60.446 | 15.061 | 0.000 | 0.000 | 0.000 | 12.124 | 0.000 | 0.000 | 12.369 | 0.000 | 0.000 | 0.000 |
| FEBRA2021339 | 0.000 | 79.225 | 0.000 | 0.000 | 0.000 | 0.000 | 0.000 | 0.000 | 0.000 | 0.000 | 0.000 | 0.000 | 0.000 |
| FEBRA2021571 | 0.000 | 100.000 | 0.000 | 0.000 | 0.000 | 0.000 | 0.000 | 0.000 | 0.000 | 0.000 | 0.000 | 0.000 | 0.000 |
| FEBRA2021908 | 0.000 | 100.000 | 0.000 | 0.000 | 0.000 | 0.000 | 0.000 | 0.000 | 0.000 | 0.000 | 0.000 | 0.000 | 0.000 |
| FEBRA2021966 | 7.969 | 10.753 | 0.000 | 3.062 | 0.000 | 0.000 | 0.000 | 0.000 | 0.000 | 0.000 | 0.000 | 0.000 | 0.000 |
| FEBRA2024136 | 0.000 | 28.600 | 0.000 | 0.000 | 0.000 | 0.000 | 0.000 | 0.000 | 0.000 | 0.000 | 42.701 | 0.000 | 0.000 |
| FEBRA2024150 | 0.000 | 55.970 | 0.000 | 0.000 | 0.000 | 0.000 | 0.000 | 0.000 | 0.000 | 0.000 | 0.000 | 0.000 | 0.000 |
| FEBRA2024343 | 0.000 | 100.000 | 0.000 | 0.000 | 0.000 | 0.000 | 0.000 | 0.000 | 0.000 | 0.000 | 0.000 | 0.000 | 0.000 |
| FEBRA2024744 | 0.000 | 100.000 | 0.000 | 0.000 | 0.000 | 0.000 | 0.000 | 0.000 | 0.000 | 0.000 | 0.000 | 0.000 | 0.000 |
| FEBRA2025427 | 4.579 | 24.717 | 3.079 | 0.000 | 0.000 | 4.995 | 0.000 | 0.000 | 0.000 | 0.000 | 9.226 | 0.000 | 0.000 |
| FEBRA2026984 | 0.000 | 43.714 | 0.000 | 12.446 | 0.000 | 0.000 | 0.000 | 0.000 | 0.000 | 0.000 | 0.000 | 0.000 | 0.000 |
| FEBRA2027082 | 0.000 | 100.000 | 0.000 | 0.000 | 0.000 | 0.000 | 0.000 | 0.000 | 0.000 | 0.000 | 0.000 | 0.000 | 0.000 |
| FEBRA2027297 | 0.000 | 100.000 | 0.000 | 0.000 | 0.000 | 0.000 | 0.000 | 0.000 | 0.000 | 0.000 | 0.000 | 0.000 | 0.000 |
| FEBRA2027352 | 0.000 | 100.000 | 0.000 | 0.000 | 0.000 | 0.000 | 0.000 | 0.000 | 0.000 | 0.000 | 0.000 | 0.000 | 0.000 |
| FEBRA2028366 | 0.000 | 100.000 | 0.000 | 0.000 | 0.000 | 0.000 | 0.000 | 0.000 | 0.000 | 0.000 | 0.000 | 0.000 | 0.000 |
| FEBRA2028477 | 0.000 | 100.000 | 0.000 | 0.000 | 0.000 | 0.000 | 0.000 | 0.000 | 0.000 | 0.000 | 0.000 | 0.000 | 0.000 |
| FEBRA2028618 | 9.389 | 12.669 | 6.313 | 10.821 | 0.000 | 5.121 | 5.082 | 0.000 | 0.000 | 0.000 | 0.000 | 0.000 | 0.000 |
| HCASM2007047 | 0.000 | 0.000 | 0.000 | 0.000 | 0.000 | 0.000 | 3.431 | 0.000 | 0.000 | 0.000 | 0.000 | 0.000 | 0.000 |
| HCHON2000028 | 0.000 | 0.000 | 0.000 | 0.000 | 0.000 | 0.000 | 0.000 | 0.000 | 5.620 | 1.619 | 0.000 | 0.000 | 5.278 |
| HCHON2000212 | 15.878 | 0.000 | 10.677 | 0.000 | 0.000 | 0.000 | 0.000 | 0.000 | 0.000 | 0.000 | 0.000 | 0.000 | 19.062 |
| HCHON2000244 | 1.215 | 4.920 | 0.000 | 0.934 | 0.000 | 0.663 | 0.658 | 0.000 | 0.000 | 0.671 | 0.000 | 0.000 | 0.000 |
| HCHON2000626 | 0.000 | 0.000 | 0.000 | 0.834 | 0.000 | 0.000 | 2.351 | 8.091 | 4.164 | 0.000 | 4.375 | 0.000 | 1.304 |
| HCHON2001084 | 0.000 | 0.000 | 0.000 | 0.000 | 0.000 | 0.000 | 0.000 | 5.268 | 0.000 | 0.000 | 0.000 | 0.000 | 2.546 |
| HCHON2001217 | 3.846 | 2.595 | 2.586 | 2.216 | 0.000 | 0.000 | 3.123 | 0.000 | 0.000 | 2.124 | 0.000 | 0.000 | 5.771 |
| HCHON2002676 | 0.000 | 0.000 | 0.000 | 0.000 | 0.000 | 0.000 | 13.647 | 0.000 | 0.000 | 0.000 | 0.000 | 0.000 | 0.000 |
| HCHON2005921 | 0.000 | 0.000 | 5.152 | 2.944 | 0.000 | 0.000 | 0.000 | 0.000 | 0.000 | 12.694 | 0.000 | 0.000 | 9.198 |
| HCHON2006250 | 0.000 | 0.000 | 0.000 | 0.000 | 0.000 | 0.000 | 1.565 | 0.000 | 0.000 | 0.000 | 0.000 | 0.000 | 1.735 |
| HCHON2007881 | 0.000 | 0.000 | 16.411 | 0.000 | 0.000 | 0.000 | 0.000 | 0.000 | 0.000 | 0.000 | 0.000 | 0.000 | 0.000 |
| HCHON2008112 | 0.000 | 0.000 | 0.000 | 0.000 | 0.000 | 13.736 | 0.000 | 0.000 | 0.000 | 0.000 | 0.000 | 0.000 | 0.000 |
| HEART1000074 | 0.000 | 0.000 | 2.829 | 1.078 | 0.000 | 0.000 | 0.759 | 1.742 | 0.000 | 0.000 | 0.000 | 0.000 | 0.000 |
| HEART2007031 | 0.000 | 0.000 | 0.000 | 0.000 | 0.000 | 0.000 | 0.000 | 0.000 | 0.000 | 11.593 | 0.000 | 0.000 | 12.601 |
| HHDPC1000118 | 15.487 | 5.225 | 0.000 | 0.000 | 0.000 | 0.000 | 2.096 | 0.000 | 0.000 | 6.415 | 0.000 | 0.000 | 0.000 |
| HLUNG2001996 | 0.000 | 0.000 | 0.000 | 0.000 | 0.000 | 0.000 | 0.000 | 0.000 | 0.000 | 0.000 | 0.000 | 0.000 | 23.216 |
| HLUNG2002465 | 0.000 | 0.000 | 0.000 | 0.000 | 0.000 | 4.875 | 1.209 | 2.775 | 4.285 | 3.702 | 0.000 | 0.000 | 1.341 |
| HLUNG2003003 | 0.000 | 0.000 | 0.000 | 0.000 | 0.000 | 0.000 | 0.000 | 0.000 | 0.000 | 16.306 | 0.000 | 0.000 | 0.000 |
| HSYRA2009075 | 0.000 | 0.000 | 0.000 | 0.000 | 0.000 | 1.212 | 0.000 | 0.000 | 0.000 | 0.000 | 0.000 | 0.000 | 0.000 |
| IMR322000127 | 8.585 | 11.585 | 1.924 | 4.398 | 5.597 | 10.926 | 3.098 | 0.000 | 5.488 | 3.161 | 0.000 | 0.000 | 0.000 |
| IMR322000917 | 43.645 | 0.000 | 14.674 | 0.000 | 0.000 | 0.000 | 0.000 | 0.000 | 0.000 | 0.000 | 0.000 | 0.000 | 0.000 |
| IMR322001380 | 7.324 | 9.883 | 0.000 | 2.814 | 7.163 | 11.984 | 0.000 | 9.096 | 0.000 | 2.022 | 0.000 | 21.443 | 2.198 |
| IMR322002035 | 0.000 | 0.000 | 0.000 | 0.000 | 0.000 | 0.000 | 36.176 | 0.000 | 0.000 | 0.000 | 0.000 | 0.000 | 0.000 |
| KIDNE2000665 | 3.576 | 0.000 | 2.405 | 1.374 | 0.000 | 1.951 | 0.000 | 0.000 | 0.000 | 0.000 | 0.000 | 0.000 | 0.000 |
| KIDNE2002252 | 0.000 | 0.000 | 2.708 | 0.000 | 0.000 | 0.000 | 0.000 | 0.000 | 0.000 | 0.000 | 0.000 | 0.000 | 2.417 |
| KIDNE2005543 | 0.000 | 0.000 | 0.000 | 0.000 | 0.000 | 0.000 | 0.000 | 0.000 | 0.000 | 18.168 | 0.000 | 0.000 | 19.748 |
| KIDNE2006580 | 0.000 | 0.000 | 0.000 | 14.933 | 0.000 | 7.066 | 7.013 | 0.000 | 0.000 | 14.310 | 0.000 | 0.000 | 7.777 |
| KIDNE2011314 | 0.000 | 0.000 | 0.000 | 0.000 | 0.000 | 0.000 | 0.000 | 0.000 | 0.000 | 0.000 | 0.000 | 0.000 | 24.133 |
| MESAN2006563 | 3.827 | 0.000 | 2.573 | 1.470 | 0.000 | 0.000 | 0.691 | 0.000 | 0.000 | 0.000 | 2.570 | 0.000 | 1.532 |
| MESAN2012054 | 4.713 | 0.000 | 3.169 | 5.432 | 0.000 | 0.000 | 12.754 | 0.000 | 0.000 | 15.615 | 0.000 | 0.000 | 0.000 |
| MESAN2015515 | 0.000 | 0.000 | 0.000 | 0.000 | 0.000 | 2.677 | 0.000 | 0.000 | 0.000 | 0.000 | 0.000 | 0.000 | 0.000 |
| MESTC1000042 | 0.000 | 0.000 | 0.000 | 0.797 | 0.000 | 0.000 | 2.245 | 0.000 | 0.000 | 0.000 | 0.000 | 0.000 | 0.000 |
| NB9N41000340 | 0.000 | 0.000 | 0.000 | 2.059 | 0.000 | 0.000 | 0.000 | 0.000 | 0.000 | 0.000 | 0.000 | 5.229 | 0.000 |
| NESOP2001752 | 0.000 | 0.000 | 0.000 | 3.228 | 0.000 | 0.000 | 0.000 | 0.000 | 0.000 | 0.000 | 0.000 | 0.000 | 0.000 |
| NHNPC2001223 | 6.894 | 0.000 | 0.000 | 0.000 | 0.000 | 0.000 | 0.000 | 0.000 | 0.000 | 0.000 | 0.000 | 0.000 | 0.000 |
| NOVAR2001783 | 0.000 | 0.000 | 0.000 | 0.000 | 0.000 | 0.000 | 4.027 | 0.000 | 0.000 | 0.000 | 0.000 | 0.000 | 0.000 |
| NT2NE2005890 | 0.000 | 0.000 | 0.000 | 0.000 | 0.000 | 35.735 | 0.000 | 0.000 | 0.000 | 0.000 | 0.000 | 0.000 | 0.000 |
| NT2NE2006909 | 0.527 | 0.711 | 0.000 | 0.000 | 0.000 | 0.287 | 0.285 | 1.964 | 0.000 | 0.000 | 0.000 | 0.000 | 0.000 |
| NT2NE2008060 | 0.000 | 0.000 | 25.528 | 0.000 | 0.000 | 0.000 | 0.000 | 0.000 | 0.000 | 0.000 | 0.000 | 0.000 | 0.000 |
| NT2RI2003993 | 0.000 | 9.464 | 0.000 | 0.000 | 0.000 | 0.000 | 0.000 | 0.000 | 0.000 | 0.000 | 0.000 | 0.000 | 0.000 |
| NT2RI2005166 | 0.000 | 0.000 | 0.000 | 18.511 | 0.000 | 0.000 | 0.000 | 0.000 | 0.000 | 0.000 | 0.000 | 0.000 | 0.000 |
| NT2RI2008724 | 3.393 | 0.000 | 0.000 | 0.000 | 0.000 | 1.850 | 1.836 | 0.000 | 0.000 | 1.874 | 0.000 | 0.000 | 4.073 |
| NT2RI2012659 | 0.000 | 0.000 | 0.000 | 48.128 | 0.000 | 13.665 | 13.562 | 0.000 | 0.000 | 0.000 | 0.000 | 0.000 | 0.000 |
| NT2RI2014733 | 0.000 | 0.000 | 0.000 | 0.000 | 0.000 | 0.000 | 24.938 | 0.000 | 0.000 | 0.000 | 0.000 | 0.000 | 0.000 |
| NT2RI2018311 | 0.000 | 0.000 | 0.000 | 0.000 | 0.000 | 0.000 | 0.000 | 0.000 | 0.000 | 21.918 | 0.000 | 0.000 | 0.000 |
| NT2RI2019751 | 0.000 | 0.000 | 0.000 | 28.087 | 0.000 | 0.000 | 0.000 | 0.000 | 0.000 | 0.000 | 0.000 | 0.000 | 0.000 |
| NT2RI3000622 | 0.000 | 21.343 | 0.000 | 0.000 | 0.000 | 0.000 | 0.000 | 0.000 | 0.000 | 0.000 | 0.000 | 0.000 | 0.000 |
| NT2RI3001515 | 0.000 | 0.000 | 40.364 | 0.000 | 0.000 | 21.825 | 0.000 | 0.000 | 0.000 | 11.050 | 0.000 | 0.000 | 0.000 |
| NT2RI3002842 | 0.000 | 0.000 | 0.000 | 0.000 | 0.000 | 0.000 | 0.000 | 0.000 | 0.000 | 0.000 | 0.000 | 0.000 | 20.781 |
| NT2RI3002892 | 0.000 | 4.387 | 0.000 | 9.992 | 0.000 | 1.773 | 8.799 | 4.037 | 0.000 | 16.159 | 0.000 | 0.000 | 13.661 |
| NT2RI3003382 | 0.000 | 0.000 | 0.000 | 28.087 | 0.000 | 0.000 | 0.000 | 0.000 | 0.000 | 0.000 | 0.000 | 0.000 | 0.000 |
| NT2RI3004510 | 0.000 | 0.000 | 0.000 | 20.009 | 0.000 | 0.000 | 0.000 | 0.000 | 0.000 | 28.761 | 0.000 | 0.000 | 0.000 |
| NT2RI3005403 | 0.000 | 0.000 | 0.000 | 0.000 | 0.000 | 0.000 | 0.000 | 0.000 | 0.000 | 0.000 | 0.000 | 0.000 | 37.898 |
| NT2RI3005724 | 0.000 | 0.000 | 0.000 | 42.065 | 0.000 | 5.972 | 0.000 | 0.000 | 0.000 | 12.093 | 0.000 | 0.000 | 0.000 |
| NT2RI3006284 | 0.000 | 0.000 | 0.000 | 1.469 | 0.000 | 0.000 | 4.138 | 0.000 | 0.000 | 0.000 | 0.000 | 0.000 | 0.000 |
| NT2RI3006673 | 0.000 | 0.000 | 0.000 | 0.000 | 0.000 | 10.055 | 19.959 | 0.000 | 0.000 | 10.182 | 0.000 | 0.000 | 0.000 |
| NT2RI3007291 | 0.000 | 0.000 | 0.000 | 35.535 | 0.000 | 0.000 | 0.000 | 0.000 | 0.000 | 12.770 | 0.000 | 0.000 | 0.000 |
| NT2RI3007543 | 0.000 | 0.000 | 3.162 | 1.204 | 0.000 | 0.855 | 1.697 | 0.000 | 0.000 | 0.866 | 0.000 | 3.059 | 0.000 |
| NT2RI3008055 | 0.000 | 0.000 | 0.000 | 15.678 | 0.000 | 0.000 | 44.179 | 0.000 | 0.000 | 0.000 | 0.000 | 0.000 | 0.000 |
| NT2RP7004123 | 7.090 | 0.000 | 0.000 | 1.362 | 0.000 | 0.000 | 0.000 | 4.403 | 0.000 | 0.000 | 0.000 | 0.000 | 2.128 |
| NT2RP7005529 | 0.000 | 0.000 | 0.000 | 5.240 | 0.000 | 7.439 | 14.766 | 0.000 | 0.000 | 15.065 | 0.000 | 0.000 | 0.000 |
| NT2RP7009030 | 17.773 | 0.000 | 35.854 | 0.000 | 0.000 | 0.000 | 0.000 | 0.000 | 0.000 | 0.000 | 0.000 | 0.000 | 0.000 |
| NT2RP7009147 | 0.000 | 0.000 | 1.979 | 0.000 | 0.000 | 0.000 | 14.337 | 0.000 | 5.643 | 1.625 | 0.000 | 0.000 | 3.533 |
| NT2RP7010599 | 0.000 | 0.000 | 0.000 | 22.750 | 0.000 | 0.000 | 0.000 | 0.000 | 0.000 | 0.000 | 0.000 | 0.000 | 0.000 |
| NT2RP7014005 | 0.000 | 0.000 | 0.000 | 0.000 | 0.000 | 0.000 | 5.818 | 0.000 | 0.000 | 0.000 | 0.000 | 0.000 | 0.000 |
| NT2RP7015512 | 11.941 | 0.000 | 0.000 | 0.000 | 0.000 | 0.000 | 0.000 | 0.000 | 0.000 | 0.000 | 0.000 | 0.000 | 0.000 |
| NT2RP7017474 | 0.000 | 0.000 | 0.000 | 13.360 | 0.000 | 0.000 | 18.823 | 0.000 | 0.000 | 0.000 | 0.000 | 0.000 | 0.000 |
| NTONG2000413 | 0.000 | 0.000 | 13.407 | 0.000 | 0.000 | 0.000 | 0.000 | 0.000 | 0.000 | 0.000 | 0.000 | 0.000 | 0.000 |
| NTONG2005969 | 0.000 | 8.008 | 0.000 | 4.560 | 11.607 | 6.473 | 0.000 | 14.740 | 0.000 | 0.000 | 0.000 | 0.000 | 3.562 |
| NTONG2008088 | 0.000 | 0.000 | 0.000 | 0.000 | 0.000 | 0.000 | 0.000 | 0.000 | 0.000 | 0.000 | 0.000 | 0.000 | 11.332 |
| OCBBF1000254 | 0.000 | 0.000 | 55.216 | 0.000 | 0.000 | 44.784 | 0.000 | 0.000 | 0.000 | 0.000 | 0.000 | 0.000 | 0.000 |
| OCBBF2001794 | 0.000 | 11.785 | 11.746 | 0.000 | 0.000 | 4.764 | 4.728 | 0.000 | 0.000 | 0.000 | 0.000 | 0.000 | 0.000 |
| OCBBF2002124 | 0.000 | 0.000 | 100.000 | 0.000 | 0.000 | 0.000 | 0.000 | 0.000 | 0.000 | 0.000 | 0.000 | 0.000 | 0.000 |
| OCBBF2003819 | 0.000 | 0.000 | 26.175 | 14.955 | 0.000 | 0.000 | 0.000 | 0.000 | 0.000 | 21.496 | 0.000 | 0.000 | 0.000 |
| OCBBF2004826 | 0.000 | 0.000 | 100.000 | 0.000 | 0.000 | 0.000 | 0.000 | 0.000 | 0.000 | 0.000 | 0.000 | 0.000 | 0.000 |
| OCBBF2004883 | 0.000 | 0.000 | 100.000 | 0.000 | 0.000 | 0.000 | 0.000 | 0.000 | 0.000 | 0.000 | 0.000 | 0.000 | 0.000 |
| OCBBF2005428 | 6.090 | 0.000 | 4.095 | 0.000 | 0.000 | 0.000 | 0.000 | 7.563 | 0.000 | 0.000 | 0.000 | 0.000 | 0.000 |
| OCBBF2006005 | 0.000 | 0.000 | 5.922 | 0.000 | 0.000 | 0.000 | 9.535 | 0.000 | 0.000 | 9.728 | 0.000 | 0.000 | 21.147 |
| OCBBF2006058 | 0.000 | 0.000 | 100.000 | 0.000 | 0.000 | 0.000 | 0.000 | 0.000 | 0.000 | 0.000 | 0.000 | 0.000 | 0.000 |
| OCBBF2006151 | 0.000 | 0.000 | 7.162 | 4.092 | 0.000 | 0.000 | 0.000 | 0.000 | 20.423 | 11.764 | 0.000 | 0.000 | 0.000 |
| OCBBF2006567 | 0.000 | 0.000 | 100.000 | 0.000 | 0.000 | 0.000 | 0.000 | 0.000 | 0.000 | 0.000 | 0.000 | 0.000 | 0.000 |
| OCBBF2006764 | 28.349 | 0.000 | 19.063 | 21.782 | 0.000 | 15.461 | 15.345 | 0.000 | 0.000 | 0.000 | 0.000 | 0.000 | 0.000 |
| OCBBF2007028 | 5.102 | 3.442 | 15.437 | 8.820 | 0.000 | 6.956 | 9.665 | 0.000 | 0.000 | 11.269 | 0.000 | 0.000 | 18.374 |
| OCBBF2007068 | 0.000 | 0.000 | 54.907 | 0.000 | 0.000 | 0.000 | 0.000 | 0.000 | 0.000 | 45.093 | 0.000 | 0.000 | 0.000 |
| OCBBF2007114 | 0.000 | 0.000 | 39.836 | 0.000 | 38.623 | 21.540 | 0.000 | 0.000 | 0.000 | 0.000 | 0.000 | 0.000 | 0.000 |
| OCBBF2007428 | 0.000 | 0.000 | 100.000 | 0.000 | 0.000 | 0.000 | 0.000 | 0.000 | 0.000 | 0.000 | 0.000 | 0.000 | 0.000 |
| OCBBF2007478 | 0.000 | 0.000 | 100.000 | 0.000 | 0.000 | 0.000 | 0.000 | 0.000 | 0.000 | 0.000 | 0.000 | 0.000 | 0.000 |
| OCBBF2007610 | 0.000 | 0.000 | 100.000 | 0.000 | 0.000 | 0.000 | 0.000 | 0.000 | 0.000 | 0.000 | 0.000 | 0.000 | 0.000 |
| OCBBF2008770 | 24.703 | 33.334 | 33.223 | 0.000 | 0.000 | 0.000 | 0.000 | 0.000 | 0.000 | 0.000 | 0.000 | 0.000 | 0.000 |
| OCBBF2009788 | 0.000 | 0.000 | 100.000 | 0.000 | 0.000 | 0.000 | 0.000 | 0.000 | 0.000 | 0.000 | 0.000 | 0.000 | 0.000 |
| OCBBF2009926 | 0.000 | 0.000 | 100.000 | 0.000 | 0.000 | 0.000 | 0.000 | 0.000 | 0.000 | 0.000 | 0.000 | 0.000 | 0.000 |
| OCBBF2010140 | 0.000 | 0.000 | 40.385 | 0.000 | 0.000 | 0.000 | 32.508 | 0.000 | 0.000 | 0.000 | 0.000 | 0.000 | 0.000 |
| OCBBF2010416 | 0.000 | 0.000 | 100.000 | 0.000 | 0.000 | 0.000 | 0.000 | 0.000 | 0.000 | 0.000 | 0.000 | 0.000 | 0.000 |
| OCBBF2017516 | 0.000 | 0.000 | 100.000 | 0.000 | 0.000 | 0.000 | 0.000 | 0.000 | 0.000 | 0.000 | 0.000 | 0.000 | 0.000 |
| OCBBF2019327 | 0.000 | 0.000 | 100.000 | 0.000 | 0.000 | 0.000 | 0.000 | 0.000 | 0.000 | 0.000 | 0.000 | 0.000 | 0.000 |
| OCBBF2019823 | 0.000 | 0.000 | 100.000 | 0.000 | 0.000 | 0.000 | 0.000 | 0.000 | 0.000 | 0.000 | 0.000 | 0.000 | 0.000 |
| OCBBF2020343 | 0.000 | 0.000 | 63.640 | 36.360 | 0.000 | 0.000 | 0.000 | 0.000 | 0.000 | 0.000 | 0.000 | 0.000 | 0.000 |
| OCBBF2020453 | 0.000 | 0.000 | 100.000 | 0.000 | 0.000 | 0.000 | 0.000 | 0.000 | 0.000 | 0.000 | 0.000 | 0.000 | 0.000 |
| OCBBF2020639 | 0.000 | 0.000 | 56.118 | 0.000 | 0.000 | 0.000 | 0.000 | 0.000 | 0.000 | 0.000 | 0.000 | 0.000 | 25.048 |
| OCBBF2020741 | 0.000 | 0.000 | 17.527 | 20.028 | 0.000 | 0.000 | 0.000 | 0.000 | 0.000 | 28.789 | 0.000 | 0.000 | 0.000 |
| OCBBF2020801 | 0.000 | 0.000 | 59.836 | 0.000 | 0.000 | 0.000 | 0.000 | 0.000 | 0.000 | 0.000 | 0.000 | 0.000 | 0.000 |
| OCBBF2020838 | 0.000 | 0.000 | 100.000 | 0.000 | 0.000 | 0.000 | 0.000 | 0.000 | 0.000 | 0.000 | 0.000 | 0.000 | 0.000 |
| OCBBF2021020 | 0.000 | 0.000 | 3.312 | 0.000 | 0.000 | 0.000 | 0.000 | 0.000 | 0.000 | 0.000 | 0.000 | 0.000 | 0.000 |
| OCBBF2021286 | 0.000 | 23.004 | 22.928 | 6.550 | 0.000 | 0.000 | 18.456 | 0.000 | 0.000 | 18.829 | 0.000 | 0.000 | 10.233 |
| OCBBF2021323 | 0.000 | 0.000 | 100.000 | 0.000 | 0.000 | 0.000 | 0.000 | 0.000 | 0.000 | 0.000 | 0.000 | 0.000 | 0.000 |
| OCBBF2021788 | 0.000 | 0.000 | 100.000 | 0.000 | 0.000 | 0.000 | 0.000 | 0.000 | 0.000 | 0.000 | 0.000 | 0.000 | 0.000 |
| OCBBF2022351 | 0.000 | 0.000 | 63.640 | 36.360 | 0.000 | 0.000 | 0.000 | 0.000 | 0.000 | 0.000 | 0.000 | 0.000 | 0.000 |
| OCBBF2022574 | 0.000 | 0.000 | 100.000 | 0.000 | 0.000 | 0.000 | 0.000 | 0.000 | 0.000 | 0.000 | 0.000 | 0.000 | 0.000 |
| OCBBF2023162 | 18.995 | 0.000 | 12.773 | 0.000 | 37.152 | 31.080 | 0.000 | 0.000 | 0.000 | 0.000 | 0.000 | 0.000 | 0.000 |
| OCBBF2023643 | 0.000 | 0.000 | 100.000 | 0.000 | 0.000 | 0.000 | 0.000 | 0.000 | 0.000 | 0.000 | 0.000 | 0.000 | 0.000 |
| OCBBF2024719 | 0.000 | 0.000 | 36.847 | 0.000 | 0.000 | 0.000 | 0.000 | 0.000 | 0.000 | 30.261 | 0.000 | 0.000 | 32.892 |
| OCBBF2024781 | 0.000 | 0.000 | 100.000 | 0.000 | 0.000 | 0.000 | 0.000 | 0.000 | 0.000 | 0.000 | 0.000 | 0.000 | 0.000 |
| OCBBF2024850 | 0.000 | 0.000 | 5.522 | 1.577 | 0.000 | 2.239 | 4.445 | 0.000 | 0.000 | 4.535 | 0.000 | 8.013 | 7.393 |
| OCBBF2025028 | 0.000 | 0.000 | 100.000 | 0.000 | 0.000 | 0.000 | 0.000 | 0.000 | 0.000 | 0.000 | 0.000 | 0.000 | 0.000 |
| OCBBF2025458 | 0.000 | 0.000 | 100.000 | 0.000 | 0.000 | 0.000 | 0.000 | 0.000 | 0.000 | 0.000 | 0.000 | 0.000 | 0.000 |
| OCBBF2025527 | 0.000 | 0.000 | 63.640 | 36.360 | 0.000 | 0.000 | 0.000 | 0.000 | 0.000 | 0.000 | 0.000 | 0.000 | 0.000 |
| OCBBF2025730 | 0.000 | 0.000 | 100.000 | 0.000 | 0.000 | 0.000 | 0.000 | 0.000 | 0.000 | 0.000 | 0.000 | 0.000 | 0.000 |
| OCBBF2026645 | 0.000 | 0.000 | 100.000 | 0.000 | 0.000 | 0.000 | 0.000 | 0.000 | 0.000 | 0.000 | 0.000 | 0.000 | 0.000 |
| OCBBF2027423 | 0.000 | 0.000 | 100.000 | 0.000 | 0.000 | 0.000 | 0.000 | 0.000 | 0.000 | 0.000 | 0.000 | 0.000 | 0.000 |
| OCBBF2027478 | 0.000 | 0.000 | 100.000 | 0.000 | 0.000 | 0.000 | 0.000 | 0.000 | 0.000 | 0.000 | 0.000 | 0.000 | 0.000 |
| OCBBF2028173 | 0.000 | 0.000 | 100.000 | 0.000 | 0.000 | 0.000 | 0.000 | 0.000 | 0.000 | 0.000 | 0.000 | 0.000 | 0.000 |
| OCBBF2028935 | 0.000 | 2.886 | 1.438 | 1.643 | 4.183 | 1.167 | 5.789 | 0.000 | 0.000 | 7.087 | 8.618 | 8.349 | 2.568 |
| OCBBF2029901 | 0.000 | 0.000 | 100.000 | 0.000 | 0.000 | 0.000 | 0.000 | 0.000 | 0.000 | 0.000 | 0.000 | 0.000 | 0.000 |
| OCBBF2030354 | 0.000 | 0.000 | 100.000 | 0.000 | 0.000 | 0.000 | 0.000 | 0.000 | 0.000 | 0.000 | 0.000 | 0.000 | 0.000 |
| OCBBF2030517 | 0.000 | 0.000 | 100.000 | 0.000 | 0.000 | 0.000 | 0.000 | 0.000 | 0.000 | 0.000 | 0.000 | 0.000 | 0.000 |
| OCBBF2030574 | 0.000 | 0.000 | 100.000 | 0.000 | 0.000 | 0.000 | 0.000 | 0.000 | 0.000 | 0.000 | 0.000 | 0.000 | 0.000 |
| OCBBF2030708 | 0.000 | 0.000 | 100.000 | 0.000 | 0.000 | 0.000 | 0.000 | 0.000 | 0.000 | 0.000 | 0.000 | 0.000 | 0.000 |
| OCBBF2031167 | 0.000 | 0.000 | 100.000 | 0.000 | 0.000 | 0.000 | 0.000 | 0.000 | 0.000 | 0.000 | 0.000 | 0.000 | 0.000 |
| OCBBF2031366 | 0.000 | 0.000 | 100.000 | 0.000 | 0.000 | 0.000 | 0.000 | 0.000 | 0.000 | 0.000 | 0.000 | 0.000 | 0.000 |
| OCBBF2032590 | 0.000 | 0.000 | 100.000 | 0.000 | 0.000 | 0.000 | 0.000 | 0.000 | 0.000 | 0.000 | 0.000 | 0.000 | 0.000 |
| OCBBF2032599 | 0.000 | 0.000 | 100.000 | 0.000 | 0.000 | 0.000 | 0.000 | 0.000 | 0.000 | 0.000 | 0.000 | 0.000 | 0.000 |
| OCBBF2032611 | 0.000 | 0.000 | 100.000 | 0.000 | 0.000 | 0.000 | 0.000 | 0.000 | 0.000 | 0.000 | 0.000 | 0.000 | 0.000 |
| OCBBF2032671 | 0.000 | 0.000 | 100.000 | 0.000 | 0.000 | 0.000 | 0.000 | 0.000 | 0.000 | 0.000 | 0.000 | 0.000 | 0.000 |
| OCBBF2033869 | 0.000 | 0.000 | 100.000 | 0.000 | 0.000 | 0.000 | 0.000 | 0.000 | 0.000 | 0.000 | 0.000 | 0.000 | 0.000 |
| OCBBF2035110 | 0.000 | 0.000 | 100.000 | 0.000 | 0.000 | 0.000 | 0.000 | 0.000 | 0.000 | 0.000 | 0.000 | 0.000 | 0.000 |
| OCBBF2035214 | 0.000 | 0.000 | 55.216 | 0.000 | 0.000 | 44.784 | 0.000 | 0.000 | 0.000 | 0.000 | 0.000 | 0.000 | 0.000 |
| OCBBF2035564 | 0.000 | 0.000 | 100.000 | 0.000 | 0.000 | 0.000 | 0.000 | 0.000 | 0.000 | 0.000 | 0.000 | 0.000 | 0.000 |
| OCBBF2035885 | 0.000 | 0.000 | 100.000 | 0.000 | 0.000 | 0.000 | 0.000 | 0.000 | 0.000 | 0.000 | 0.000 | 0.000 | 0.000 |
| OCBBF2035916 | 0.000 | 0.000 | 100.000 | 0.000 | 0.000 | 0.000 | 0.000 | 0.000 | 0.000 | 0.000 | 0.000 | 0.000 | 0.000 |
| OCBBF2036476 | 0.000 | 0.000 | 100.000 | 0.000 | 0.000 | 0.000 | 0.000 | 0.000 | 0.000 | 0.000 | 0.000 | 0.000 | 0.000 |
| OCBBF2036743 | 0.000 | 0.000 | 27.462 | 15.690 | 0.000 | 11.137 | 11.053 | 0.000 | 0.000 | 0.000 | 0.000 | 0.000 | 12.257 |
| OCBBF2037068 | 0.000 | 0.000 | 18.587 | 0.000 | 0.000 | 0.000 | 0.000 | 0.000 | 0.000 | 0.000 | 0.000 | 0.000 | 0.000 |
| OCBBF2037340 | 0.000 | 0.000 | 100.000 | 0.000 | 0.000 | 0.000 | 0.000 | 0.000 | 0.000 | 0.000 | 0.000 | 0.000 | 0.000 |
| OCBBF2037398 | 0.000 | 0.000 | 100.000 | 0.000 | 0.000 | 0.000 | 0.000 | 0.000 | 0.000 | 0.000 | 0.000 | 0.000 | 0.000 |
| OCBBF2037547 | 0.000 | 0.000 | 100.000 | 0.000 | 0.000 | 0.000 | 0.000 | 0.000 | 0.000 | 0.000 | 0.000 | 0.000 | 0.000 |
| OCBBF2037598 | 0.000 | 0.000 | 100.000 | 0.000 | 0.000 | 0.000 | 0.000 | 0.000 | 0.000 | 0.000 | 0.000 | 0.000 | 0.000 |
| OCBBF2037638 | 0.000 | 0.000 | 100.000 | 0.000 | 0.000 | 0.000 | 0.000 | 0.000 | 0.000 | 0.000 | 0.000 | 0.000 | 0.000 |
| OCBBF2038317 | 0.000 | 0.000 | 12.245 | 0.000 | 0.000 | 0.000 | 19.713 | 0.000 | 0.000 | 0.000 | 0.000 | 0.000 | 10.930 |
| OCBBF3000296 | 0.000 | 0.000 | 47.069 | 0.000 | 0.000 | 0.000 | 0.000 | 0.000 | 0.000 | 19.328 | 0.000 | 0.000 | 0.000 |
| OCBBF3000483 | 7.373 | 0.000 | 9.916 | 14.163 | 0.000 | 4.021 | 11.973 | 0.000 | 0.000 | 8.143 | 0.000 | 0.000 | 0.000 |
| OCBBF3002553 | 0.000 | 0.000 | 100.000 | 0.000 | 0.000 | 0.000 | 0.000 | 0.000 | 0.000 | 0.000 | 0.000 | 0.000 | 0.000 |
| OCBBF3002600 | 0.000 | 0.000 | 100.000 | 0.000 | 0.000 | 0.000 | 0.000 | 0.000 | 0.000 | 0.000 | 0.000 | 0.000 | 0.000 |
| OCBBF3003320 | 59.793 | 0.000 | 40.207 | 0.000 | 0.000 | 0.000 | 0.000 | 0.000 | 0.000 | 0.000 | 0.000 | 0.000 | 0.000 |
| OCBBF3003592 | 0.000 | 0.000 | 100.000 | 0.000 | 0.000 | 0.000 | 0.000 | 0.000 | 0.000 | 0.000 | 0.000 | 0.000 | 0.000 |
| OCBBF3004314 | 0.000 | 0.000 | 100.000 | 0.000 | 0.000 | 0.000 | 0.000 | 0.000 | 0.000 | 0.000 | 0.000 | 0.000 | 0.000 |
| OCBBF3006802 | 0.000 | 0.000 | 100.000 | 0.000 | 0.000 | 0.000 | 0.000 | 0.000 | 0.000 | 0.000 | 0.000 | 0.000 | 0.000 |
| OCBBF3007516 | 0.000 | 0.000 | 4.698 | 2.684 | 0.000 | 0.000 | 0.000 | 0.000 | 0.000 | 0.000 | 0.000 | 0.000 | 0.000 |
| OCBBF3008230 | 0.000 | 0.000 | 37.069 | 0.000 | 0.000 | 0.000 | 29.840 | 0.000 | 0.000 | 0.000 | 0.000 | 0.000 | 33.091 |
| OCBBF3009279 | 0.000 | 0.000 | 100.000 | 0.000 | 0.000 | 0.000 | 0.000 | 0.000 | 0.000 | 0.000 | 0.000 | 0.000 | 0.000 |
| PEBLM2004666 | 0.000 | 0.000 | 0.000 | 0.000 | 0.000 | 3.744 | 3.715 | 0.000 | 0.000 | 0.000 | 0.000 | 0.000 | 0.000 |
| PERIC2000889 | 0.000 | 0.000 | 0.000 | 6.314 | 0.000 | 8.963 | 0.000 | 0.000 | 0.000 | 0.000 | 0.000 | 0.000 | 0.000 |
| PERIC2002766 | 0.000 | 0.000 | 15.571 | 0.000 | 0.000 | 0.000 | 0.000 | 0.000 | 0.000 | 0.000 | 0.000 | 0.000 | 0.000 |
| PERIC2003720 | 0.000 | 0.000 | 0.000 | 0.000 | 0.000 | 13.012 | 0.000 | 0.000 | 0.000 | 0.000 | 0.000 | 0.000 | 0.000 |
| PLACE6001185 | 0.000 | 0.000 | 0.000 | 15.159 | 0.000 | 0.000 | 21.358 | 0.000 | 0.000 | 0.000 | 0.000 | 0.000 | 0.000 |
| PLACE6019385 | 0.000 | 0.000 | 5.039 | 0.000 | 0.000 | 0.000 | 0.000 | 0.000 | 0.000 | 0.000 | 0.000 | 0.000 | 0.000 |
| PUAEN2002489 | 24.108 | 0.000 | 8.106 | 9.262 | 0.000 | 0.000 | 0.000 | 0.000 | 0.000 | 0.000 | 0.000 | 0.000 | 21.707 |
| PUAEN2005930 | 0.000 | 0.000 | 0.000 | 0.000 | 0.000 | 0.000 | 18.362 | 0.000 | 0.000 | 9.367 | 0.000 | 0.000 | 10.181 |
| PUAEN2006701 | 0.000 | 0.000 | 0.000 | 0.000 | 8.128 | 0.000 | 2.249 | 0.000 | 15.938 | 2.295 | 0.000 | 0.000 | 0.000 |
| PUAEN2007044 | 0.000 | 0.000 | 3.561 | 5.087 | 0.000 | 1.444 | 8.600 | 0.000 | 0.000 | 0.000 | 0.000 | 0.000 | 1.590 |
| PUAEN2009174 | 0.000 | 0.000 | 0.000 | 0.000 | 0.000 | 2.856 | 0.000 | 0.000 | 0.000 | 0.000 | 0.000 | 0.000 | 0.000 |
| PUAEN2009655 | 0.000 | 0.000 | 0.000 | 0.000 | 0.000 | 0.000 | 18.275 | 0.000 | 0.000 | 7.458 | 0.000 | 0.000 | 0.000 |
| RECTM2001347 | 0.000 | 0.000 | 5.153 | 0.000 | 0.000 | 0.000 | 0.000 | 0.000 | 0.000 | 0.000 | 0.000 | 0.000 | 4.600 |
| SKMUS2000757 | 0.000 | 0.000 | 0.000 | 0.000 | 0.000 | 0.000 | 0.000 | 0.000 | 0.000 | 0.000 | 0.000 | 0.000 | 5.480 |
| SKNMC2002402 | 0.000 | 0.000 | 9.158 | 0.000 | 0.000 | 7.428 | 0.000 | 0.000 | 0.000 | 0.000 | 0.000 | 0.000 | 0.000 |
| SKNSH2000482 | 0.000 | 0.000 | 0.000 | 0.000 | 0.000 | 9.821 | 0.000 | 0.000 | 0.000 | 0.000 | 0.000 | 0.000 | 0.000 |
| SMINT2001818 | 0.000 | 0.000 | 0.000 | 0.000 | 3.387 | 0.000 | 0.000 | 0.000 | 0.000 | 0.000 | 0.000 | 0.000 | 0.000 |
| SPLEN2001599 | 0.000 | 17.788 | 0.000 | 0.000 | 0.000 | 7.190 | 0.000 | 0.000 | 0.000 | 0.000 | 0.000 | 0.000 | 0.000 |
| SPLEN2002467 | 6.019 | 0.000 | 4.047 | 0.000 | 0.000 | 6.565 | 0.000 | 0.000 | 0.000 | 0.000 | 0.000 | 0.000 | 0.000 |
| SPLEN2006122 | 0.000 | 2.099 | 0.000 | 0.000 | 0.000 | 0.000 | 0.000 | 0.000 | 0.000 | 0.000 | 0.000 | 0.000 | 0.934 |
| SPLEN2010912 | 0.000 | 0.000 | 15.386 | 26.371 | 0.000 | 0.000 | 0.000 | 0.000 | 0.000 | 12.636 | 0.000 | 0.000 | 13.734 |
| SPLEN2012624 | 0.000 | 0.000 | 0.000 | 7.178 | 0.000 | 0.000 | 0.000 | 0.000 | 0.000 | 10.317 | 0.000 | 0.000 | 0.000 |
| SPLEN2025491 | 0.000 | 0.000 | 0.000 | 0.000 | 0.000 | 0.000 | 0.000 | 0.000 | 0.000 | 0.000 | 0.000 | 0.000 | 38.930 |
| SPLEN2027268 | 0.000 | 50.440 | 0.000 | 14.361 | 0.000 | 0.000 | 0.000 | 0.000 | 0.000 | 0.000 | 0.000 | 0.000 | 0.000 |
| SPLEN2028914 | 0.000 | 0.000 | 3.469 | 0.000 | 0.000 | 0.000 | 2.792 | 0.000 | 0.000 | 0.000 | 0.000 | 0.000 | 0.000 |
| SPLEN2029912 | 0.000 | 0.000 | 0.000 | 0.000 | 0.000 | 1.646 | 0.000 | 0.000 | 0.000 | 0.000 | 0.000 | 0.000 | 0.000 |
| SPLEN2031424 | 0.000 | 0.000 | 0.000 | 5.405 | 0.000 | 0.000 | 15.229 | 0.000 | 0.000 | 0.000 | 0.000 | 0.000 | 8.444 |
| SPLEN2031547 | 0.000 | 0.000 | 11.735 | 5.587 | 5.689 | 4.759 | 1.574 | 0.000 | 0.000 | 11.244 | 0.000 | 0.000 | 1.746 |
| SPLEN2032154 | 0.000 | 0.000 | 0.000 | 0.000 | 0.000 | 0.000 | 0.000 | 0.000 | 0.000 | 0.000 | 0.000 | 0.000 | 38.930 |
| SPLEN2034781 | 0.000 | 0.000 | 0.000 | 0.000 | 0.000 | 0.000 | 27.984 | 0.000 | 0.000 | 0.000 | 0.000 | 0.000 | 0.000 |
| SPLEN2036821 | 0.000 | 0.000 | 0.000 | 0.000 | 0.000 | 0.000 | 0.000 | 0.000 | 0.000 | 0.000 | 0.000 | 0.000 | 38.930 |
| SPLEN2036932 | 0.000 | 21.926 | 0.000 | 2.081 | 0.000 | 0.000 | 2.932 | 6.726 | 31.158 | 5.982 | 0.000 | 0.000 | 0.000 |
| SPLEN2037194 | 0.000 | 0.000 | 0.000 | 28.977 | 0.000 | 0.000 | 0.000 | 0.000 | 0.000 | 0.000 | 0.000 | 0.000 | 0.000 |
| SPLEN2038345 | 0.000 | 0.000 | 0.000 | 0.000 | 0.000 | 29.627 | 0.000 | 0.000 | 0.000 | 0.000 | 0.000 | 0.000 | 0.000 |
| SPLEN2042303 | 0.000 | 0.000 | 32.557 | 0.000 | 0.000 | 0.000 | 0.000 | 0.000 | 0.000 | 0.000 | 0.000 | 0.000 | 0.000 |
| SYNOV1000374 | 0.000 | 0.000 | 0.000 | 0.000 | 0.000 | 0.000 | 0.000 | 0.000 | 0.000 | 0.000 | 0.000 | 0.000 | 22.005 |
| SYNOV2005216 | 0.000 | 0.000 | 12.130 | 0.000 | 0.000 | 0.000 | 0.000 | 0.000 | 0.000 | 0.000 | 0.000 | 0.000 | 0.000 |
| SYNOV2014400 | 0.000 | 0.000 | 0.000 | 0.000 | 0.000 | 0.000 | 12.977 | 0.000 | 0.000 | 13.240 | 0.000 | 0.000 | 0.000 |
| SYNOV4002346 | 0.000 | 0.000 | 0.000 | 0.000 | 0.000 | 0.000 | 5.997 | 0.000 | 0.000 | 0.000 | 0.000 | 0.000 | 0.000 |
| SYNOV4002883 | 0.000 | 0.000 | 0.000 | 0.000 | 0.000 | 0.000 | 23.940 | 0.000 | 0.000 | 24.425 | 0.000 | 0.000 | 0.000 |
| SYNOV4007430 | 0.000 | 0.000 | 0.000 | 0.000 | 0.000 | 0.000 | 31.677 | 0.000 | 0.000 | 0.000 | 0.000 | 0.000 | 0.000 |
| SYNOV4007671 | 1.354 | 1.827 | 0.000 | 0.520 | 2.649 | 0.739 | 0.000 | 0.000 | 0.000 | 0.000 | 0.000 | 0.000 | 0.813 |
| SYNOV4008440 | 0.000 | 7.355 | 1.833 | 2.094 | 0.000 | 0.000 | 1.475 | 3.384 | 0.000 | 3.010 | 0.000 | 0.000 | 3.272 |
| TESOP2001605 | 0.000 | 0.000 | 12.130 | 0.000 | 0.000 | 0.000 | 0.000 | 0.000 | 0.000 | 0.000 | 0.000 | 0.000 | 0.000 |
| TESOP2002273 | 7.498 | 0.000 | 15.126 | 2.881 | 14.666 | 0.000 | 0.000 | 0.000 | 0.000 | 0.000 | 0.000 | 0.000 | 0.000 |
| TESOP2002451 | 4.388 | 0.000 | 5.902 | 3.372 | 0.000 | 0.000 | 2.375 | 0.000 | 0.000 | 0.000 | 0.000 | 0.000 | 0.000 |
| TESOP2002950 | 0.000 | 0.000 | 0.000 | 9.399 | 0.000 | 0.000 | 0.000 | 0.000 | 0.000 | 0.000 | 0.000 | 0.000 | 0.000 |
| TESTI1000330 | 0.000 | 0.000 | 0.000 | 52.057 | 0.000 | 0.000 | 0.000 | 0.000 | 0.000 | 0.000 | 0.000 | 0.000 | 0.000 |
| TESTI2003573 | 0.000 | 0.000 | 0.000 | 0.000 | 0.000 | 0.000 | 0.000 | 0.000 | 0.000 | 0.000 | 0.000 | 26.931 | 0.000 |
| TESTI2009474 | 0.000 | 0.000 | 7.530 | 0.000 | 0.000 | 2.036 | 0.000 | 0.000 | 0.000 | 0.000 | 0.000 | 0.000 | 0.000 |
| TESTI2049246 | 0.000 | 0.000 | 0.000 | 0.000 | 0.000 | 0.000 | 0.000 | 0.000 | 0.000 | 53.653 | 0.000 | 0.000 | 29.159 |
| TESTI4000014 | 1.980 | 0.890 | 2.440 | 2.028 | 0.000 | 1.799 | 1.964 | 0.000 | 0.000 | 2.551 | 0.665 | 0.644 | 1.188 |
| TESTI4000209 | 0.000 | 0.000 | 3.291 | 0.000 | 0.000 | 2.669 | 2.649 | 0.000 | 0.000 | 0.000 | 0.000 | 0.000 | 2.938 |
| TESTI4000349 | 0.000 | 0.000 | 0.000 | 36.899 | 0.000 | 0.000 | 0.000 | 0.000 | 0.000 | 0.000 | 0.000 | 0.000 | 0.000 |
| TESTI4001100 | 7.570 | 10.215 | 10.181 | 8.725 | 0.000 | 4.129 | 4.098 | 0.000 | 0.000 | 4.180 | 0.000 | 0.000 | 4.544 |
| TESTI4001561 | 0.000 | 0.000 | 0.000 | 28.578 | 0.000 | 0.000 | 0.000 | 0.000 | 0.000 | 0.000 | 0.000 | 0.000 | 0.000 |
| TESTI4002290 | 0.000 | 0.000 | 0.000 | 0.000 | 0.000 | 0.000 | 0.000 | 0.000 | 0.000 | 0.000 | 0.000 | 0.000 | 62.915 |
| TESTI4002647 | 0.000 | 0.000 | 65.522 | 0.000 | 0.000 | 0.000 | 0.000 | 0.000 | 0.000 | 0.000 | 0.000 | 0.000 | 0.000 |
| TESTI4005857 | 0.000 | 79.225 | 0.000 | 0.000 | 0.000 | 0.000 | 0.000 | 0.000 | 0.000 | 0.000 | 0.000 | 0.000 | 0.000 |
| TESTI4006137 | 0.000 | 0.000 | 0.000 | 0.000 | 0.000 | 0.000 | 25.755 | 0.000 | 0.000 | 0.000 | 0.000 | 0.000 | 28.561 |
| TESTI4006326 | 0.000 | 0.000 | 48.724 | 0.000 | 0.000 | 0.000 | 0.000 | 0.000 | 0.000 | 0.000 | 0.000 | 0.000 | 0.000 |
| TESTI4008797 | 0.000 | 0.000 | 0.000 | 0.000 | 0.000 | 0.000 | 12.429 | 0.000 | 0.000 | 12.681 | 0.000 | 0.000 | 27.567 |
| TESTI4009286 | 1.339 | 0.000 | 0.000 | 0.000 | 0.000 | 0.000 | 1.450 | 0.000 | 0.000 | 1.479 | 0.000 | 0.000 | 0.804 |
| TESTI4010377 | 0.000 | 0.000 | 0.000 | 0.000 | 0.000 | 0.000 | 0.000 | 0.000 | 0.000 | 60.949 | 0.000 | 0.000 | 0.000 |
| TESTI4010851 | 5.050 | 6.814 | 4.075 | 2.716 | 0.000 | 2.203 | 3.280 | 0.000 | 0.000 | 3.904 | 2.035 | 0.000 | 3.031 |
| TESTI4010928 | 0.000 | 0.000 | 0.000 | 0.000 | 0.000 | 0.000 | 0.000 | 0.000 | 0.000 | 34.221 | 0.000 | 0.000 | 0.000 |
| TESTI4011161 | 0.000 | 0.000 | 26.175 | 14.955 | 0.000 | 0.000 | 0.000 | 0.000 | 0.000 | 21.496 | 0.000 | 0.000 | 0.000 |
| TESTI4012702 | 0.000 | 0.000 | 2.708 | 0.000 | 0.000 | 0.000 | 0.000 | 0.000 | 0.000 | 0.000 | 0.000 | 0.000 | 2.417 |
| TESTI4013675 | 0.000 | 0.000 | 0.000 | 14.015 | 0.000 | 0.000 | 0.000 | 0.000 | 0.000 | 0.000 | 0.000 | 0.000 | 0.000 |
| TESTI4013817 | 0.000 | 0.000 | 0.000 | 0.000 | 0.000 | 27.369 | 27.163 | 0.000 | 0.000 | 27.712 | 0.000 | 0.000 | 0.000 |
| TESTI4014159 | 0.000 | 0.000 | 0.000 | 16.638 | 0.000 | 11.810 | 0.000 | 0.000 | 0.000 | 23.916 | 0.000 | 0.000 | 25.996 |
| TESTI4014175 | 49.352 | 0.000 | 33.186 | 0.000 | 0.000 | 0.000 | 0.000 | 0.000 | 0.000 | 0.000 | 0.000 | 0.000 | 0.000 |
| TESTI4014306 | 0.000 | 0.000 | 0.000 | 52.057 | 0.000 | 0.000 | 0.000 | 0.000 | 0.000 | 0.000 | 0.000 | 0.000 | 0.000 |
| TESTI4014694 | 0.000 | 0.000 | 0.000 | 0.791 | 0.000 | 0.000 | 2.229 | 0.000 | 0.000 | 0.000 | 0.000 | 0.000 | 0.000 |
| TESTI4014818 | 0.000 | 0.000 | 0.000 | 0.000 | 0.000 | 0.000 | 0.000 | 0.000 | 0.000 | 60.949 | 0.000 | 0.000 | 0.000 |
| TESTI4019843 | 73.865 | 0.000 | 0.000 | 0.000 | 0.000 | 0.000 | 0.000 | 0.000 | 0.000 | 0.000 | 0.000 | 0.000 | 0.000 |
| TESTI4021478 | 0.000 | 0.000 | 0.000 | 0.000 | 0.000 | 0.000 | 22.098 | 0.000 | 0.000 | 0.000 | 0.000 | 0.000 | 49.012 |
| TESTI4022936 | 0.000 | 0.000 | 0.000 | 0.000 | 0.000 | 26.646 | 26.445 | 0.000 | 0.000 | 0.000 | 0.000 | 0.000 | 0.000 |
| TESTI4024420 | 0.000 | 0.000 | 0.000 | 0.000 | 0.000 | 0.000 | 37.398 | 0.000 | 0.000 | 38.155 | 0.000 | 0.000 | 0.000 |
| TESTI4027821 | 0.000 | 0.000 | 0.000 | 0.000 | 0.000 | 0.000 | 60.471 | 0.000 | 0.000 | 0.000 | 0.000 | 0.000 | 0.000 |
| TESTI4029836 | 0.000 | 0.000 | 0.000 | 0.000 | 0.000 | 60.652 | 0.000 | 0.000 | 0.000 | 0.000 | 0.000 | 0.000 | 0.000 |
| TESTI4037156 | 2.556 | 1.380 | 0.688 | 0.982 | 0.000 | 1.394 | 0.000 | 0.000 | 0.980 | 0.282 | 2.060 | 0.998 | 0.307 |
| TESTI4037188 | 0.000 | 0.000 | 0.000 | 0.000 | 0.000 | 60.652 | 0.000 | 0.000 | 0.000 | 0.000 | 0.000 | 0.000 | 0.000 |
| TESTI4046819 | 0.000 | 0.000 | 0.000 | 14.013 | 0.000 | 0.000 | 0.000 | 0.000 | 0.000 | 0.000 | 0.000 | 0.000 | 0.000 |
| THYMU2001090 | 0.000 | 0.000 | 0.000 | 0.000 | 0.000 | 0.000 | 21.252 | 0.000 | 0.000 | 0.000 | 0.000 | 0.000 | 47.135 |
| THYMU2011736 | 0.000 | 74.935 | 0.000 | 0.000 | 0.000 | 0.000 | 0.000 | 0.000 | 0.000 | 0.000 | 0.000 | 0.000 | 0.000 |
| THYMU2014353 | 0.000 | 0.000 | 0.000 | 0.000 | 0.000 | 37.663 | 0.000 | 0.000 | 0.000 | 0.000 | 0.000 | 0.000 | 0.000 |
| THYMU2016204 | 0.000 | 0.000 | 59.836 | 0.000 | 0.000 | 0.000 | 0.000 | 0.000 | 0.000 | 0.000 | 0.000 | 0.000 | 0.000 |
| THYMU2016523 | 0.000 | 0.000 | 0.000 | 23.416 | 0.000 | 0.000 | 0.000 | 0.000 | 0.000 | 0.000 | 0.000 | 0.000 | 0.000 |
| THYMU2023967 | 0.000 | 0.000 | 0.000 | 43.363 | 0.000 | 0.000 | 0.000 | 0.000 | 0.000 | 31.165 | 0.000 | 0.000 | 0.000 |
| THYMU2025707 | 0.000 | 0.000 | 0.000 | 0.000 | 0.000 | 0.000 | 0.000 | 0.000 | 0.000 | 9.440 | 0.000 | 0.000 | 20.521 |
| THYMU2030264 | 0.000 | 0.000 | 0.000 | 45.981 | 0.000 | 0.000 | 0.000 | 0.000 | 0.000 | 0.000 | 0.000 | 0.000 | 0.000 |
| THYMU2031341 | 0.000 | 0.000 | 4.286 | 0.000 | 0.000 | 0.000 | 0.000 | 0.000 | 0.000 | 7.040 | 0.000 | 0.000 | 0.000 |
| THYMU2031890 | 0.000 | 0.000 | 0.000 | 0.000 | 0.000 | 0.000 | 0.000 | 0.000 | 0.000 | 0.000 | 0.000 | 64.019 | 0.000 |
| THYMU2032696 | 0.000 | 0.000 | 0.000 | 0.000 | 0.000 | 0.000 | 0.000 | 2.411 | 0.000 | 0.000 | 0.000 | 3.789 | 0.000 |
| THYMU2032825 | 0.000 | 0.000 | 11.550 | 0.000 | 0.000 | 0.000 | 0.000 | 0.000 | 0.000 | 0.000 | 0.000 | 0.000 | 20.620 |
| THYMU2033308 | 0.000 | 0.000 | 0.000 | 0.000 | 0.000 | 0.000 | 13.964 | 0.000 | 0.000 | 0.000 | 0.000 | 0.000 | 0.000 |
| THYMU2033787 | 0.000 | 0.000 | 0.000 | 0.000 | 0.000 | 0.000 | 0.000 | 0.000 | 0.000 | 0.000 | 0.000 | 0.000 | 57.080 |
| THYMU2034374 | 23.239 | 0.000 | 0.000 | 0.000 | 0.000 | 0.000 | 0.000 | 0.000 | 0.000 | 0.000 | 0.000 | 0.000 | 0.000 |
| THYMU2035735 | 0.000 | 0.000 | 0.000 | 0.936 | 0.000 | 0.000 | 1.319 | 3.027 | 0.000 | 2.692 | 0.000 | 0.000 | 0.000 |
| THYMU2037226 | 0.000 | 0.000 | 0.000 | 0.000 | 0.000 | 17.265 | 0.000 | 0.000 | 0.000 | 34.964 | 0.000 | 0.000 | 0.000 |
| THYMU2039315 | 0.000 | 0.000 | 0.000 | 0.000 | 0.000 | 0.000 | 54.530 | 0.000 | 0.000 | 0.000 | 0.000 | 0.000 | 0.000 |
| THYMU2039780 | 0.000 | 0.000 | 0.000 | 45.981 | 0.000 | 0.000 | 0.000 | 0.000 | 0.000 | 0.000 | 0.000 | 0.000 | 0.000 |
| THYMU2040975 | 0.000 | 0.000 | 2.182 | 0.000 | 0.000 | 0.000 | 0.000 | 0.000 | 0.000 | 0.000 | 0.000 | 0.000 | 1.948 |
| THYMU3001083 | 0.000 | 0.000 | 0.000 | 45.981 | 0.000 | 0.000 | 0.000 | 0.000 | 0.000 | 0.000 | 0.000 | 0.000 | 0.000 |
| THYMU3001234 | 0.000 | 0.000 | 0.000 | 7.868 | 0.000 | 0.000 | 11.085 | 25.432 | 0.000 | 0.000 | 0.000 | 0.000 | 12.293 |
| THYMU3001379 | 0.000 | 0.000 | 0.000 | 0.000 | 0.000 | 25.376 | 0.000 | 0.000 | 0.000 | 25.695 | 0.000 | 0.000 | 27.929 |
| THYMU3003309 | 0.000 | 0.000 | 36.117 | 20.635 | 0.000 | 0.000 | 0.000 | 0.000 | 0.000 | 0.000 | 0.000 | 0.000 | 0.000 |
| THYMU3004835 | 0.000 | 0.000 | 0.000 | 0.000 | 0.000 | 19.793 | 0.000 | 0.000 | 0.000 | 20.041 | 0.000 | 0.000 | 21.784 |
| THYMU3006485 | 0.000 | 0.000 | 0.000 | 45.981 | 0.000 | 0.000 | 0.000 | 0.000 | 0.000 | 0.000 | 0.000 | 0.000 | 0.000 |
| THYMU3007137 | 0.000 | 0.000 | 0.000 | 0.000 | 0.000 | 0.000 | 0.000 | 0.000 | 0.000 | 37.956 | 0.000 | 0.000 | 0.000 |
| THYMU3008171 | 0.000 | 0.000 | 0.000 | 14.316 | 0.000 | 0.000 | 20.170 | 0.000 | 0.000 | 20.579 | 0.000 | 0.000 | 0.000 |
| TKIDN2009641 | 21.363 | 0.000 | 7.182 | 4.104 | 0.000 | 5.826 | 5.782 | 0.000 | 0.000 | 0.000 | 21.519 | 0.000 | 12.823 |
| TKIDN2009889 | 0.000 | 0.000 | 0.000 | 0.000 | 0.000 | 0.000 | 35.077 | 0.000 | 0.000 | 0.000 | 0.000 | 0.000 | 0.000 |
| TKIDN2010934 | 0.000 | 0.000 | 25.127 | 0.000 | 0.000 | 0.000 | 0.000 | 0.000 | 0.000 | 0.000 | 0.000 | 0.000 | 0.000 |
| TKIDN2013287 | 0.000 | 0.000 | 21.501 | 0.000 | 0.000 | 0.000 | 0.000 | 0.000 | 0.000 | 0.000 | 0.000 | 0.000 | 0.000 |
| TKIDN2015788 | 0.000 | 0.000 | 0.000 | 0.000 | 9.505 | 5.301 | 5.261 | 0.000 | 0.000 | 2.684 | 0.000 | 0.000 | 2.917 |
| TLIVE2001327 | 0.000 | 0.000 | 0.000 | 0.000 | 0.000 | 0.000 | 0.000 | 0.000 | 0.000 | 0.000 | 0.000 | 0.000 | 5.201 |
| TLIVE2004320 | 0.000 | 0.000 | 0.000 | 0.000 | 0.000 | 12.783 | 0.000 | 0.000 | 0.000 | 0.000 | 0.000 | 0.000 | 0.000 |
| TRACH1000205 | 0.000 | 0.000 | 4.889 | 8.380 | 0.000 | 3.965 | 19.677 | 0.000 | 0.000 | 4.015 | 0.000 | 0.000 | 0.000 |
| TRACH2001443 | 17.167 | 0.000 | 0.000 | 0.000 | 0.000 | 0.000 | 0.000 | 0.000 | 0.000 | 0.000 | 0.000 | 0.000 | 0.000 |
| TRACH2001549 | 0.000 | 0.000 | 0.000 | 0.000 | 0.000 | 4.260 | 8.457 | 0.000 | 0.000 | 21.569 | 0.000 | 0.000 | 4.689 |
| TRACH2001684 | 0.000 | 0.000 | 0.000 | 0.000 | 0.000 | 17.948 | 0.000 | 0.000 | 0.000 | 0.000 | 0.000 | 0.000 | 0.000 |
| TRACH2005811 | 1.948 | 7.887 | 0.000 | 0.000 | 0.000 | 0.000 | 2.109 | 2.419 | 0.000 | 2.152 | 0.000 | 0.000 | 3.508 |
| TRACH2006049 | 0.000 | 0.000 | 0.000 | 0.000 | 0.000 | 0.000 | 47.167 | 0.000 | 0.000 | 0.000 | 0.000 | 0.000 | 0.000 |
| TRACH2007834 | 0.000 | 1.257 | 0.627 | 0.000 | 0.000 | 1.016 | 0.504 | 0.000 | 0.000 | 0.515 | 1.877 | 0.000 | 0.559 |
| TRACH2008300 | 3.136 | 0.000 | 6.327 | 3.615 | 6.135 | 11.974 | 10.186 | 3.895 | 6.014 | 13.857 | 0.000 | 12.243 | 11.296 |
| TRACH2023299 | 0.000 | 0.000 | 0.000 | 0.000 | 0.000 | 0.000 | 0.000 | 0.000 | 0.000 | 0.000 | 0.000 | 0.000 | 14.157 |
| TRACH2025344 | 0.000 | 0.000 | 0.000 | 0.000 | 0.000 | 19.846 | 0.000 | 0.000 | 0.000 | 0.000 | 0.000 | 0.000 | 0.000 |
| TRACH2025535 | 3.218 | 0.000 | 0.000 | 2.472 | 0.000 | 0.000 | 5.806 | 1.332 | 0.000 | 2.369 | 0.000 | 0.000 | 1.288 |
| TRACH2025911 | 0.000 | 0.000 | 0.000 | 0.000 | 0.000 | 47.355 | 0.000 | 0.000 | 0.000 | 0.000 | 0.000 | 0.000 | 0.000 |
| TRACH3000014 | 0.000 | 0.000 | 0.000 | 0.000 | 0.000 | 0.000 | 0.000 | 0.000 | 0.000 | 18.547 | 0.000 | 0.000 | 0.000 |
| TRACH3001427 | 8.576 | 4.629 | 11.534 | 3.295 | 3.355 | 3.742 | 5.571 | 2.130 | 3.289 | 2.842 | 0.000 | 3.348 | 1.030 |
| TRACH3002192 | 2.304 | 3.109 | 8.522 | 3.541 | 2.253 | 1.885 | 4.989 | 2.861 | 0.000 | 6.999 | 2.321 | 0.000 | 2.766 |
| TRACH3004068 | 0.000 | 3.553 | 0.000 | 0.000 | 5.150 | 1.436 | 0.000 | 0.000 | 0.000 | 0.000 | 0.000 | 5.139 | 1.581 |
| TRACH3004721 | 0.000 | 0.000 | 3.611 | 4.127 | 0.000 | 5.858 | 8.721 | 6.669 | 0.000 | 2.966 | 0.000 | 0.000 | 3.224 |
| TRACH3005294 | 0.000 | 0.000 | 4.614 | 0.000 | 0.000 | 0.000 | 7.428 | 0.000 | 0.000 | 0.000 | 0.000 | 0.000 | 0.000 |
| TRACH3006038 | 0.000 | 0.000 | 0.000 | 38.787 | 0.000 | 0.000 | 0.000 | 0.000 | 0.000 | 0.000 | 0.000 | 0.000 | 0.000 |
| TRACH3006412 | 0.000 | 0.000 | 0.000 | 38.787 | 0.000 | 0.000 | 0.000 | 0.000 | 0.000 | 0.000 | 0.000 | 0.000 | 0.000 |
| TRACH3007479 | 0.000 | 0.000 | 0.000 | 0.000 | 0.000 | 1.084 | 1.075 | 0.000 | 0.000 | 0.000 | 0.000 | 0.000 | 0.000 |
| TRACH3008093 | 0.000 | 0.000 | 9.128 | 3.477 | 0.000 | 0.000 | 2.449 | 5.619 | 0.000 | 0.000 | 0.000 | 0.000 | 2.716 |
| TRACH3009455 | 0.000 | 0.000 | 0.000 | 0.000 | 0.000 | 0.000 | 47.167 | 0.000 | 0.000 | 0.000 | 0.000 | 0.000 | 0.000 |
| TSTOM1000135 | 0.000 | 0.000 | 0.000 | 0.000 | 0.000 | 0.000 | 0.000 | 0.000 | 0.000 | 0.000 | 0.000 | 0.000 | 9.361 |
| TUTER1000122 | 0.000 | 0.000 | 8.109 | 0.000 | 0.000 | 0.000 | 0.000 | 0.000 | 0.000 | 3.330 | 0.000 | 0.000 | 0.000 |
| TUTER2000904 | 5.158 | 0.000 | 0.000 | 5.944 | 0.000 | 2.813 | 0.000 | 0.000 | 0.000 | 5.697 | 0.000 | 0.000 | 3.096 |
| UTERU2002410 | 0.000 | 0.000 | 0.000 | 0.417 | 0.000 | 0.592 | 0.000 | 2.694 | 2.080 | 0.000 | 0.000 | 2.117 | 1.302 |
| UTERU2004929 | 21.446 | 0.000 | 0.000 | 0.000 | 0.000 | 11.697 | 0.000 | 0.000 | 0.000 | 11.843 | 0.000 | 0.000 | 0.000 |
| UTERU2005621 | 0.000 | 5.619 | 0.000 | 0.000 | 8.145 | 0.000 | 0.000 | 0.000 | 15.971 | 0.000 | 0.000 | 8.128 | 0.000 |
| UTERU2006115 | 0.000 | 0.000 | 0.000 | 5.563 | 0.000 | 0.000 | 7.837 | 0.000 | 0.000 | 0.000 | 0.000 | 0.000 | 17.383 |
| UTERU2007520 | 0.000 | 16.720 | 8.332 | 4.761 | 0.000 | 6.758 | 0.000 | 0.000 | 23.761 | 0.000 | 0.000 | 0.000 | 0.000 |
| UTERU2014678 | 0.000 | 0.000 | 51.019 | 0.000 | 0.000 | 0.000 | 0.000 | 0.000 | 0.000 | 0.000 | 0.000 | 0.000 | 0.000 |
| UTERU2019706 | 0.000 | 0.000 | 0.000 | 0.000 | 0.000 | 0.000 | 45.606 | 0.000 | 0.000 | 0.000 | 0.000 | 0.000 | 0.000 |
| UTERU2019940 | 0.000 | 0.000 | 0.000 | 0.000 | 0.000 | 0.000 | 0.000 | 0.000 | 0.000 | 0.000 | 0.000 | 0.000 | 48.181 |
| UTERU2021163 | 0.000 | 0.000 | 0.000 | 0.000 | 0.000 | 0.000 | 0.000 | 0.000 | 0.000 | 46.104 | 0.000 | 0.000 | 0.000 |
| UTERU2023039 | 0.000 | 0.000 | 0.000 | 0.000 | 0.000 | 0.000 | 45.606 | 0.000 | 0.000 | 0.000 | 0.000 | 0.000 | 0.000 |
| UTERU2023175 | 8.252 | 0.000 | 1.850 | 0.000 | 0.000 | 0.000 | 0.000 | 0.000 | 0.000 | 0.000 | 0.000 | 5.368 | 1.651 |
| UTERU2026203 | 0.000 | 0.000 | 0.000 | 0.000 | 0.000 | 0.000 | 45.606 | 0.000 | 0.000 | 0.000 | 0.000 | 0.000 | 0.000 |
| UTERU2030213 | 0.000 | 0.000 | 0.000 | 0.000 | 0.000 | 0.000 | 0.000 | 0.000 | 0.000 | 25.133 | 0.000 | 0.000 | 0.000 |
| UTERU2030280 | 0.000 | 0.000 | 0.000 | 0.000 | 0.000 | 0.000 | 0.000 | 0.000 | 0.000 | 0.000 | 0.000 | 0.000 | 48.181 |
| UTERU3000226 | 0.000 | 0.000 | 0.000 | 37.308 | 0.000 | 0.000 | 0.000 | 0.000 | 0.000 | 0.000 | 0.000 | 0.000 | 0.000 |
| UTERU3000899 | 0.000 | 0.000 | 35.054 | 0.000 | 0.000 | 0.000 | 0.000 | 0.000 | 0.000 | 0.000 | 0.000 | 0.000 | 31.292 |
| UTERU3001571 | 0.000 | 0.000 | 0.000 | 0.000 | 0.000 | 0.000 | 0.000 | 0.000 | 0.000 | 0.000 | 0.000 | 0.000 | 48.181 |
| UTERU3001572 | 0.000 | 9.102 | 2.268 | 2.591 | 0.000 | 1.839 | 0.000 | 0.000 | 12.934 | 1.862 | 0.000 | 0.000 | 6.073 |
| UTERU3001766 | 0.000 | 0.000 | 0.000 | 0.000 | 0.000 | 45.794 | 0.000 | 0.000 | 0.000 | 0.000 | 0.000 | 0.000 | 0.000 |
| UTERU3003135 | 0.000 | 0.000 | 0.000 | 0.000 | 0.000 | 0.000 | 0.000 | 0.000 | 0.000 | 33.486 | 0.000 | 0.000 | 0.000 |
| UTERU3004709 | 0.000 | 0.000 | 0.000 | 0.000 | 0.000 | 0.000 | 0.000 | 0.000 | 0.000 | 0.000 | 0.000 | 0.000 | 48.181 |
| UTERU3005230 | 0.000 | 0.000 | 0.000 | 17.332 | 0.000 | 0.000 | 24.419 | 0.000 | 0.000 | 0.000 | 0.000 | 0.000 | 0.000 |
| UTERU3005460 | 0.000 | 0.000 | 0.000 | 37.308 | 0.000 | 0.000 | 0.000 | 0.000 | 0.000 | 0.000 | 0.000 | 0.000 | 0.000 |
| UTERU3005907 | 0.000 | 0.000 | 0.000 | 0.000 | 0.000 | 0.000 | 0.000 | 0.000 | 0.000 | 0.000 | 0.000 | 0.000 | 14.514 |
| UTERU3005970 | 0.000 | 0.000 | 0.000 | 37.308 | 0.000 | 0.000 | 0.000 | 0.000 | 0.000 | 0.000 | 0.000 | 0.000 | 0.000 |
| UTERU3006308 | 0.000 | 0.000 | 0.000 | 37.308 | 0.000 | 0.000 | 0.000 | 0.000 | 0.000 | 0.000 | 0.000 | 0.000 | 0.000 |
| UTERU3007419 | 0.000 | 0.000 | 0.000 | 37.308 | 0.000 | 0.000 | 0.000 | 0.000 | 0.000 | 0.000 | 0.000 | 0.000 | 0.000 |
| UTERU3007640 | 0.000 | 0.000 | 0.000 | 0.000 | 0.000 | 0.000 | 45.606 | 0.000 | 0.000 | 0.000 | 0.000 | 0.000 | 0.000 |
| UTERU3007913 | 0.000 | 0.000 | 0.000 | 12.951 | 0.000 | 0.000 | 0.000 | 0.000 | 0.000 | 0.000 | 0.000 | 0.000 | 0.000 |
| UTERU3009259 | 0.000 | 0.000 | 0.000 | 0.000 | 0.000 | 0.000 | 0.000 | 0.000 | 0.000 | 46.104 | 0.000 | 0.000 | 0.000 |
| UTERU3009517 | 0.000 | 0.000 | 0.000 | 0.000 | 0.000 | 45.794 | 0.000 | 0.000 | 0.000 | 0.000 | 0.000 | 0.000 | 0.000 |
| UTERU3009871 | 0.000 | 0.000 | 0.000 | 0.000 | 0.000 | 0.000 | 36.230 | 0.000 | 0.000 | 18.482 | 0.000 | 0.000 | 0.000 |
| ADRGL2000042 | 0.000 | 0.000 | 0.000 | 7.210 | 0.000 | 5.118 | 2.540 | 0.000 | 0.000 | 0.000 | 18.905 | 0.000 | 0.000 |
| BLADE2006830 | 0.000 | 0.000 | 0.000 | 2.386 | 0.000 | 0.000 | 1.681 | 0.000 | 0.000 | 0.000 | 0.000 | 0.000 | 0.000 |
| BRACE2002589 | 0.000 | 0.000 | 0.000 | 100.000 | 0.000 | 0.000 | 0.000 | 0.000 | 0.000 | 0.000 | 0.000 | 0.000 | 0.000 |
| BRACE2003609 | 0.000 | 0.000 | 11.292 | 6.452 | 0.000 | 18.317 | 9.090 | 0.000 | 0.000 | 0.000 | 0.000 | 0.000 | 0.000 |
| BRACE2009318 | 0.000 | 0.000 | 0.000 | 100.000 | 0.000 | 0.000 | 0.000 | 0.000 | 0.000 | 0.000 | 0.000 | 0.000 | 0.000 |
| BRACE2011677 | 0.000 | 0.000 | 0.000 | 100.000 | 0.000 | 0.000 | 0.000 | 0.000 | 0.000 | 0.000 | 0.000 | 0.000 | 0.000 |
| BRACE2029396 | 0.000 | 0.000 | 0.000 | 100.000 | 0.000 | 0.000 | 0.000 | 0.000 | 0.000 | 0.000 | 0.000 | 0.000 | 0.000 |
| BRACE2037299 | 0.000 | 0.000 | 0.000 | 100.000 | 0.000 | 0.000 | 0.000 | 0.000 | 0.000 | 0.000 | 0.000 | 0.000 | 0.000 |
| BRACE2039823 | 0.000 | 0.000 | 0.000 | 41.329 | 0.000 | 58.671 | 0.000 | 0.000 | 0.000 | 0.000 | 0.000 | 0.000 | 0.000 |
| BRACE2039832 | 0.000 | 0.000 | 0.000 | 100.000 | 0.000 | 0.000 | 0.000 | 0.000 | 0.000 | 0.000 | 0.000 | 0.000 | 0.000 |
| BRACE2043105 | 0.000 | 0.000 | 0.000 | 100.000 | 0.000 | 0.000 | 0.000 | 0.000 | 0.000 | 0.000 | 0.000 | 0.000 | 0.000 |
| BRACE3001058 | 0.000 | 0.000 | 0.000 | 100.000 | 0.000 | 0.000 | 0.000 | 0.000 | 0.000 | 0.000 | 0.000 | 0.000 | 0.000 |
| BRACE3001113 | 48.624 | 0.000 | 32.696 | 18.680 | 0.000 | 0.000 | 0.000 | 0.000 | 0.000 | 0.000 | 0.000 | 0.000 | 0.000 |
| BRACE3003026 | 0.000 | 0.000 | 0.000 | 11.590 | 0.000 | 0.000 | 0.000 | 0.000 | 0.000 | 0.000 | 0.000 | 0.000 | 0.000 |
| BRACE3003053 | 0.000 | 0.000 | 63.640 | 36.360 | 0.000 | 0.000 | 0.000 | 0.000 | 0.000 | 0.000 | 0.000 | 0.000 | 0.000 |
| BRACE3009127 | 0.000 | 0.000 | 0.000 | 100.000 | 0.000 | 0.000 | 0.000 | 0.000 | 0.000 | 0.000 | 0.000 | 0.000 | 0.000 |
| BRACE3010076 | 0.000 | 0.000 | 0.000 | 100.000 | 0.000 | 0.000 | 0.000 | 0.000 | 0.000 | 0.000 | 0.000 | 0.000 | 0.000 |
| BRACE3015829 | 0.000 | 0.000 | 0.000 | 100.000 | 0.000 | 0.000 | 0.000 | 0.000 | 0.000 | 0.000 | 0.000 | 0.000 | 0.000 |
| BRACE3021148 | 0.000 | 0.000 | 0.000 | 100.000 | 0.000 | 0.000 | 0.000 | 0.000 | 0.000 | 0.000 | 0.000 | 0.000 | 0.000 |
| BRALZ2017844 | 0.000 | 0.000 | 0.000 | 0.000 | 49.396 | 0.000 | 0.000 | 0.000 | 0.000 | 0.000 | 0.000 | 0.000 | 0.000 |
| BRAMY2019111 | 0.000 | 0.000 | 0.000 | 0.000 | 0.000 | 30.516 | 0.000 | 69.484 | 0.000 | 0.000 | 0.000 | 0.000 | 0.000 |
| BRAMY2035070 | 0.000 | 0.000 | 0.000 | 0.000 | 0.000 | 54.718 | 0.000 | 0.000 | 0.000 | 0.000 | 0.000 | 0.000 | 0.000 |
| BRAMY2035449 | 0.000 | 0.000 | 0.000 | 0.000 | 0.000 | 100.000 | 0.000 | 0.000 | 0.000 | 0.000 | 0.000 | 0.000 | 0.000 |
| BRAMY2035718 | 0.000 | 0.000 | 0.000 | 0.000 | 0.000 | 100.000 | 0.000 | 0.000 | 0.000 | 0.000 | 0.000 | 0.000 | 0.000 |
| BRAMY2038516 | 0.000 | 0.000 | 0.000 | 0.000 | 0.000 | 12.770 | 0.000 | 87.230 | 0.000 | 0.000 | 0.000 | 0.000 | 0.000 |
| BRAMY2039341 | 0.000 | 0.000 | 0.000 | 0.000 | 0.000 | 100.000 | 0.000 | 0.000 | 0.000 | 0.000 | 0.000 | 0.000 | 0.000 |
| BRAMY2040159 | 0.000 | 0.000 | 0.000 | 0.000 | 0.000 | 100.000 | 0.000 | 0.000 | 0.000 | 0.000 | 0.000 | 0.000 | 0.000 |
| BRAMY2041434 | 0.000 | 0.000 | 0.000 | 0.000 | 0.000 | 100.000 | 0.000 | 0.000 | 0.000 | 0.000 | 0.000 | 0.000 | 0.000 |
| BRAMY2045471 | 0.000 | 0.000 | 0.000 | 0.000 | 0.000 | 100.000 | 0.000 | 0.000 | 0.000 | 0.000 | 0.000 | 0.000 | 0.000 |
| BRAMY3004800 | 0.000 | 0.000 | 0.000 | 0.000 | 0.000 | 15.340 | 38.061 | 0.000 | 0.000 | 46.598 | 0.000 | 0.000 | 0.000 |
| BRAWH1000369 | 0.000 | 0.000 | 0.000 | 0.000 | 0.000 | 0.000 | 100.000 | 0.000 | 0.000 | 0.000 | 0.000 | 0.000 | 0.000 |
| BRAWH2006207 | 0.000 | 0.000 | 0.000 | 9.187 | 0.000 | 0.000 | 12.943 | 29.695 | 0.000 | 0.000 | 48.175 | 0.000 | 0.000 |
| BRAWH2006395 | 0.000 | 0.000 | 0.000 | 0.000 | 0.000 | 0.000 | 12.446 | 0.000 | 0.000 | 0.000 | 0.000 | 0.000 | 0.000 |
| BRAWH2008993 | 0.000 | 0.000 | 0.000 | 0.000 | 0.000 | 50.189 | 49.811 | 0.000 | 0.000 | 0.000 | 0.000 | 0.000 | 0.000 |
| BRAWH2009393 | 0.000 | 0.000 | 0.000 | 0.000 | 0.000 | 0.000 | 100.000 | 0.000 | 0.000 | 0.000 | 0.000 | 0.000 | 0.000 |
| BRAWH2010552 | 0.000 | 0.000 | 0.000 | 41.512 | 0.000 | 0.000 | 58.488 | 0.000 | 0.000 | 0.000 | 0.000 | 0.000 | 0.000 |
| BRAWH3007441 | 0.000 | 0.000 | 0.000 | 0.000 | 0.000 | 0.000 | 100.000 | 0.000 | 0.000 | 0.000 | 0.000 | 0.000 | 0.000 |
| BRAWH3009017 | 0.000 | 0.000 | 0.000 | 0.000 | 0.000 | 0.000 | 100.000 | 0.000 | 0.000 | 0.000 | 0.000 | 0.000 | 0.000 |
| BRCAN2002473 | 0.000 | 0.000 | 0.000 | 0.000 | 0.000 | 0.000 | 0.000 | 100.000 | 0.000 | 0.000 | 0.000 | 0.000 | 0.000 |
| BRCAN2002854 | 0.000 | 0.000 | 0.000 | 0.000 | 0.000 | 0.000 | 0.000 | 100.000 | 0.000 | 0.000 | 0.000 | 0.000 | 0.000 |
| BRCAN2003070 | 0.000 | 0.000 | 0.000 | 0.000 | 0.000 | 0.000 | 0.000 | 100.000 | 0.000 | 0.000 | 0.000 | 0.000 | 0.000 |
| BRCAN2014229 | 0.000 | 0.000 | 0.000 | 0.000 | 0.000 | 0.000 | 0.000 | 100.000 | 0.000 | 0.000 | 0.000 | 0.000 | 0.000 |
| BRCOC2019841 | 0.000 | 0.000 | 0.000 | 16.691 | 0.000 | 0.000 | 0.000 | 0.000 | 83.309 | 0.000 | 0.000 | 0.000 | 0.000 |
| BRHIP2002722 | 0.000 | 0.000 | 0.000 | 0.000 | 0.000 | 0.000 | 0.000 | 0.000 | 0.000 | 100.000 | 0.000 | 0.000 | 0.000 |
| BRHIP2003272 | 0.000 | 0.000 | 0.000 | 0.000 | 0.000 | 0.000 | 0.000 | 0.000 | 0.000 | 100.000 | 0.000 | 0.000 | 0.000 |
| BRHIP2005271 | 0.000 | 0.000 | 0.000 | 0.000 | 0.000 | 7.137 | 7.083 | 0.000 | 0.000 | 14.453 | 0.000 | 0.000 | 7.855 |
| BRHIP2005724 | 0.000 | 0.000 | 0.000 | 0.000 | 0.000 | 0.000 | 0.000 | 0.000 | 0.000 | 100.000 | 0.000 | 0.000 | 0.000 |
| BRHIP2006617 | 0.000 | 0.000 | 0.000 | 0.000 | 0.000 | 0.000 | 0.000 | 0.000 | 0.000 | 100.000 | 0.000 | 0.000 | 0.000 |
| BRHIP2008389 | 0.000 | 0.000 | 0.000 | 0.000 | 0.000 | 0.000 | 0.000 | 0.000 | 0.000 | 100.000 | 0.000 | 0.000 | 0.000 |
| BRHIP2012360 | 0.000 | 0.000 | 0.000 | 0.000 | 0.000 | 0.000 | 0.000 | 0.000 | 0.000 | 100.000 | 0.000 | 0.000 | 0.000 |
| BRHIP2017553 | 0.000 | 0.000 | 0.000 | 0.000 | 0.000 | 0.000 | 0.000 | 0.000 | 0.000 | 100.000 | 0.000 | 0.000 | 0.000 |
| BRHIP2026877 | 0.000 | 0.000 | 0.000 | 0.000 | 0.000 | 0.000 | 0.000 | 69.219 | 0.000 | 30.781 | 0.000 | 0.000 | 0.000 |
| BRHIP3000017 | 8.146 | 0.000 | 5.478 | 0.000 | 0.000 | 0.000 | 8.819 | 0.000 | 15.620 | 4.499 | 0.000 | 0.000 | 0.000 |
| BRHIP3000240 | 0.000 | 0.000 | 0.000 | 67.607 | 0.000 | 0.000 | 0.000 | 0.000 | 0.000 | 32.393 | 0.000 | 0.000 | 0.000 |
| BRHIP3008314 | 0.000 | 0.000 | 0.000 | 0.000 | 0.000 | 0.000 | 0.000 | 0.000 | 0.000 | 100.000 | 0.000 | 0.000 | 0.000 |
| BRHIP3026052 | 0.000 | 0.000 | 0.000 | 0.000 | 54.140 | 0.000 | 0.000 | 0.000 | 0.000 | 45.860 | 0.000 | 0.000 | 0.000 |
| BRSTN2013354 | 0.000 | 0.000 | 0.000 | 0.000 | 0.000 | 0.000 | 0.000 | 0.000 | 0.000 | 0.000 | 0.000 | 100.000 | 0.000 |
| BRTHA2002133 | 0.000 | 0.000 | 0.000 | 0.000 | 0.000 | 0.000 | 0.000 | 0.000 | 0.000 | 0.000 | 0.000 | 0.000 | 100.000 |
| BRTHA2002702 | 0.000 | 0.000 | 0.000 | 0.000 | 0.000 | 0.000 | 0.000 | 0.000 | 0.000 | 0.000 | 0.000 | 0.000 | 100.000 |
| BRTHA2007060 | 0.000 | 0.000 | 0.000 | 0.000 | 0.000 | 0.000 | 0.000 | 0.000 | 0.000 | 0.000 | 0.000 | 0.000 | 100.000 |
| BRTHA2010033 | 0.000 | 0.000 | 0.000 | 0.000 | 0.000 | 0.000 | 0.000 | 0.000 | 0.000 | 0.000 | 0.000 | 0.000 | 100.000 |
| BRTHA2011321 | 0.000 | 0.000 | 0.000 | 0.000 | 0.000 | 0.000 | 0.000 | 0.000 | 0.000 | 0.000 | 0.000 | 0.000 | 100.000 |
| BRTHA2013426 | 0.000 | 0.000 | 0.000 | 0.000 | 0.000 | 0.000 | 0.000 | 0.000 | 0.000 | 0.000 | 0.000 | 0.000 | 100.000 |
| BRTHA2013610 | 0.000 | 0.000 | 0.000 | 0.000 | 0.000 | 0.000 | 0.000 | 0.000 | 0.000 | 0.000 | 0.000 | 0.000 | 100.000 |
| BRTHA2016318 | 0.000 | 0.000 | 0.000 | 0.000 | 0.000 | 0.000 | 0.000 | 0.000 | 0.000 | 0.000 | 0.000 | 0.000 | 100.000 |
| BRTHA2017364 | 0.000 | 0.000 | 0.000 | 0.000 | 0.000 | 0.000 | 0.000 | 0.000 | 0.000 | 0.000 | 0.000 | 0.000 | 100.000 |
| BRTHA2017972 | 0.000 | 0.000 | 0.000 | 0.000 | 0.000 | 0.000 | 0.000 | 0.000 | 0.000 | 0.000 | 0.000 | 0.000 | 37.427 |
| BRTHA2018011 | 0.000 | 0.000 | 0.000 | 0.000 | 0.000 | 0.000 | 0.000 | 0.000 | 0.000 | 0.000 | 0.000 | 0.000 | 100.000 |
| BRTHA2018443 | 0.000 | 0.000 | 0.000 | 0.000 | 0.000 | 0.000 | 22.098 | 0.000 | 0.000 | 0.000 | 0.000 | 0.000 | 49.012 |
| BRTHA3000296 | 0.000 | 0.000 | 0.000 | 0.000 | 0.000 | 0.000 | 0.000 | 0.000 | 0.000 | 0.000 | 0.000 | 0.000 | 100.000 |
| BRTHA3003000 | 0.000 | 0.000 | 0.000 | 0.000 | 0.000 | 0.000 | 17.150 | 0.000 | 0.000 | 0.000 | 63.832 | 0.000 | 19.019 |
| BRTHA3008826 | 0.000 | 0.000 | 0.000 | 0.000 | 0.000 | 0.000 | 0.000 | 0.000 | 0.000 | 0.000 | 0.000 | 0.000 | 100.000 |
| CTONG2008721 | 0.000 | 0.000 | 0.000 | 0.000 | 0.000 | 0.000 | 0.000 | 0.000 | 0.000 | 0.000 | 0.000 | 0.000 | 37.320 |
| CTONG2020374 | 0.000 | 0.000 | 0.000 | 11.030 | 0.000 | 0.000 | 31.081 | 0.000 | 0.000 | 0.000 | 0.000 | 0.000 | 0.000 |
| CTONG2020378 | 0.000 | 0.000 | 0.000 | 0.000 | 0.000 | 0.000 | 16.140 | 0.000 | 0.000 | 16.467 | 0.000 | 0.000 | 0.000 |
| CTONG2020411 | 12.719 | 0.000 | 0.000 | 0.000 | 0.000 | 0.000 | 0.000 | 0.000 | 0.000 | 0.000 | 0.000 | 0.000 | 0.000 |
| CTONG2024031 | 0.000 | 0.000 | 0.000 | 3.669 | 0.000 | 0.000 | 2.584 | 0.000 | 0.000 | 0.000 | 0.000 | 0.000 | 0.000 |
| CTONG3004726 | 0.000 | 0.000 | 21.039 | 0.000 | 0.000 | 0.000 | 0.000 | 0.000 | 0.000 | 17.278 | 0.000 | 0.000 | 0.000 |
| FCBBF1000509 | 6.894 | 0.000 | 0.000 | 0.000 | 0.000 | 13.160 | 3.732 | 17.123 | 19.830 | 5.711 | 6.945 | 6.728 | 6.208 |
| FCBBF3010361 | 100.000 | 0.000 | 0.000 | 0.000 | 0.000 | 0.000 | 0.000 | 0.000 | 0.000 | 0.000 | 0.000 | 0.000 | 0.000 |
| FCBBF3027854 | 14.460 | 0.000 | 0.000 | 0.000 | 0.000 | 0.000 | 0.000 | 0.000 | 0.000 | 0.000 | 0.000 | 0.000 | 0.000 |
| FEBRA2000790 | 0.000 | 100.000 | 0.000 | 0.000 | 0.000 | 0.000 | 0.000 | 0.000 | 0.000 | 0.000 | 0.000 | 0.000 | 0.000 |
| FEBRA2001990 | 0.000 | 9.046 | 4.508 | 15.453 | 0.000 | 7.313 | 18.144 | 16.650 | 0.000 | 7.404 | 0.000 | 0.000 | 8.048 |
| FEBRA2006519 | 0.000 | 29.644 | 0.000 | 25.320 | 0.000 | 11.982 | 11.891 | 0.000 | 0.000 | 0.000 | 0.000 | 0.000 | 0.000 |
| FEBRA2008692 | 0.000 | 6.116 | 0.000 | 0.000 | 0.000 | 0.000 | 0.000 | 0.000 | 0.000 | 0.000 | 0.000 | 0.000 | 0.000 |
| FEBRA2014122 | 0.000 | 66.741 | 33.259 | 0.000 | 0.000 | 0.000 | 0.000 | 0.000 | 0.000 | 0.000 | 0.000 | 0.000 | 0.000 |
| FEBRA2027609 | 0.000 | 100.000 | 0.000 | 0.000 | 0.000 | 0.000 | 0.000 | 0.000 | 0.000 | 0.000 | 0.000 | 0.000 | 0.000 |
| FEBRA2028516 | 0.000 | 2.851 | 2.842 | 1.624 | 0.000 | 2.305 | 8.007 | 0.000 | 0.000 | 4.668 | 0.000 | 0.000 | 1.268 |
| HCASM2003018 | 0.000 | 0.000 | 10.964 | 0.000 | 0.000 | 0.000 | 0.000 | 0.000 | 0.000 | 0.000 | 0.000 | 0.000 | 0.000 |
| HCHON2000508 | 0.000 | 0.000 | 0.464 | 0.000 | 0.000 | 0.376 | 0.000 | 0.000 | 0.000 | 0.000 | 0.000 | 0.000 | 0.000 |
| HCHON2000743 | 11.280 | 0.000 | 7.585 | 8.667 | 0.000 | 0.000 | 6.105 | 0.000 | 0.000 | 0.000 | 0.000 | 0.000 | 6.771 |
| HCHON2004858 | 0.000 | 0.000 | 16.411 | 0.000 | 0.000 | 0.000 | 0.000 | 0.000 | 0.000 | 0.000 | 0.000 | 0.000 | 0.000 |
| HSYRA2005628 | 0.000 | 0.000 | 0.000 | 0.000 | 0.000 | 0.000 | 0.000 | 0.000 | 0.000 | 0.000 | 0.000 | 0.000 | 7.437 |
| IMR322001879 | 0.000 | 0.000 | 0.000 | 0.000 | 0.000 | 0.000 | 9.638 | 0.000 | 0.000 | 9.833 | 0.000 | 0.000 | 0.000 |
| NT2RI2009583 | 0.000 | 0.000 | 0.000 | 5.399 | 0.808 | 0.902 | 0.224 | 1.540 | 0.793 | 0.228 | 1.665 | 1.613 | 1.489 |
| NT2RP8000521 | 0.000 | 0.000 | 0.000 | 37.067 | 0.000 | 0.000 | 0.000 | 0.000 | 0.000 | 0.000 | 0.000 | 0.000 | 0.000 |
| OCBBF2003327 | 0.000 | 0.000 | 100.000 | 0.000 | 0.000 | 0.000 | 0.000 | 0.000 | 0.000 | 0.000 | 0.000 | 0.000 | 0.000 |
| OCBBF2005433 | 0.000 | 0.000 | 100.000 | 0.000 | 0.000 | 0.000 | 0.000 | 0.000 | 0.000 | 0.000 | 0.000 | 0.000 | 0.000 |
| OCBBF2006987 | 0.000 | 0.000 | 16.058 | 0.000 | 0.000 | 0.000 | 0.000 | 0.000 | 0.000 | 13.187 | 0.000 | 0.000 | 0.000 |
| OCBBF2008144 | 0.000 | 0.000 | 7.165 | 4.094 | 0.000 | 2.906 | 5.768 | 0.000 | 0.000 | 2.942 | 0.000 | 0.000 | 3.198 |
| OCBBF2009583 | 0.000 | 0.000 | 100.000 | 0.000 | 0.000 | 0.000 | 0.000 | 0.000 | 0.000 | 0.000 | 0.000 | 0.000 | 0.000 |
| OCBBF2011669 | 0.000 | 0.000 | 63.640 | 36.360 | 0.000 | 0.000 | 0.000 | 0.000 | 0.000 | 0.000 | 0.000 | 0.000 | 0.000 |
| OCBBF2019684 | 0.000 | 0.000 | 100.000 | 0.000 | 0.000 | 0.000 | 0.000 | 0.000 | 0.000 | 0.000 | 0.000 | 0.000 | 0.000 |
| OCBBF2020048 | 0.000 | 0.000 | 65.522 | 0.000 | 0.000 | 0.000 | 0.000 | 0.000 | 0.000 | 0.000 | 0.000 | 0.000 | 0.000 |
| OCBBF2030116 | 0.000 | 0.000 | 54.907 | 0.000 | 0.000 | 0.000 | 0.000 | 0.000 | 0.000 | 45.093 | 0.000 | 0.000 | 0.000 |
| OCBBF2032274 | 0.000 | 0.000 | 100.000 | 0.000 | 0.000 | 0.000 | 0.000 | 0.000 | 0.000 | 0.000 | 0.000 | 0.000 | 0.000 |
| OCBBF2034637 | 0.000 | 0.000 | 100.000 | 0.000 | 0.000 | 0.000 | 0.000 | 0.000 | 0.000 | 0.000 | 0.000 | 0.000 | 0.000 |
| OCBBF3002654 | 0.000 | 0.000 | 100.000 | 0.000 | 0.000 | 0.000 | 0.000 | 0.000 | 0.000 | 0.000 | 0.000 | 0.000 | 0.000 |
| OCBBF3003761 | 0.000 | 0.000 | 100.000 | 0.000 | 0.000 | 0.000 | 0.000 | 0.000 | 0.000 | 0.000 | 0.000 | 0.000 | 0.000 |
| OCBBF3004972 | 0.000 | 0.000 | 100.000 | 0.000 | 0.000 | 0.000 | 0.000 | 0.000 | 0.000 | 0.000 | 0.000 | 0.000 | 0.000 |
| PERIC2007068 | 0.000 | 0.000 | 3.834 | 8.761 | 0.000 | 0.000 | 3.086 | 7.080 | 0.000 | 4.723 | 0.000 | 0.000 | 1.711 |
| PUAEN2006335 | 0.000 | 0.000 | 15.755 | 0.000 | 0.000 | 0.000 | 12.682 | 0.000 | 0.000 | 0.000 | 0.000 | 0.000 | 0.000 |
| SPLEN2016932 | 0.000 | 0.000 | 0.000 | 0.000 | 0.000 | 0.000 | 0.000 | 0.000 | 0.000 | 0.000 | 0.000 | 0.000 | 38.930 |
| SPLEN2039379 | 0.000 | 7.220 | 10.793 | 10.278 | 0.000 | 11.672 | 5.792 | 0.000 | 0.000 | 8.864 | 0.000 | 0.000 | 3.212 |
| SYNOV2006620 | 0.000 | 0.000 | 0.000 | 0.000 | 0.000 | 0.000 | 0.000 | 0.000 | 0.000 | 0.000 | 0.000 | 0.000 | 33.957 |
| SYNOV2021953 | 0.000 | 0.000 | 0.000 | 6.793 | 0.000 | 0.000 | 0.000 | 0.000 | 0.000 | 0.000 | 0.000 | 0.000 | 0.000 |
| TESTI1000266 | 0.000 | 0.000 | 65.522 | 0.000 | 0.000 | 0.000 | 0.000 | 0.000 | 0.000 | 0.000 | 0.000 | 0.000 | 0.000 |
| TESTI2015626 | 0.608 | 0.000 | 0.409 | 0.467 | 0.000 | 0.331 | 0.000 | 0.754 | 1.165 | 0.336 | 1.224 | 2.372 | 0.000 |
| TESTI2026647 | 0.000 | 0.000 | 0.000 | 0.000 | 0.000 | 23.811 | 0.000 | 0.000 | 0.000 | 0.000 | 0.000 | 0.000 | 0.000 |
| TESTI4000214 | 0.000 | 0.000 | 0.000 | 0.000 | 0.000 | 0.000 | 0.000 | 0.000 | 0.000 | 7.979 | 0.000 | 0.000 | 0.000 |
| TESTI4001984 | 0.000 | 0.000 | 0.000 | 0.000 | 0.000 | 0.000 | 60.471 | 0.000 | 0.000 | 0.000 | 0.000 | 0.000 | 0.000 |
| TESTI4008058 | 0.000 | 0.000 | 21.899 | 0.000 | 0.000 | 0.000 | 8.814 | 20.221 | 0.000 | 0.000 | 0.000 | 31.781 | 0.000 |
| TESTI4013894 | 0.000 | 0.000 | 0.000 | 19.881 | 0.000 | 14.112 | 0.000 | 0.000 | 0.000 | 14.289 | 0.000 | 0.000 | 0.000 |
| TESTI4015442 | 0.000 | 0.000 | 25.047 | 0.000 | 0.000 | 0.000 | 0.000 | 0.000 | 0.000 | 0.000 | 0.000 | 0.000 | 0.000 |
| TESTI4017714 | 0.000 | 0.000 | 48.724 | 0.000 | 0.000 | 0.000 | 0.000 | 0.000 | 0.000 | 0.000 | 0.000 | 0.000 | 0.000 |
| TESTI4025268 | 0.000 | 0.000 | 0.000 | 0.000 | 0.000 | 0.000 | 60.471 | 0.000 | 0.000 | 0.000 | 0.000 | 0.000 | 0.000 |
| TESTI4025547 | 0.000 | 0.000 | 0.000 | 0.000 | 0.000 | 0.000 | 0.000 | 0.000 | 0.000 | 60.949 | 0.000 | 0.000 | 0.000 |
| TESTI4026207 | 0.000 | 0.000 | 0.000 | 0.000 | 0.000 | 0.000 | 0.000 | 0.000 | 0.000 | 60.949 | 0.000 | 0.000 | 0.000 |
| TESTI4032090 | 0.000 | 0.000 | 0.000 | 0.000 | 0.000 | 0.000 | 60.471 | 0.000 | 0.000 | 0.000 | 0.000 | 0.000 | 0.000 |
| THYMU2004284 | 0.000 | 0.000 | 0.000 | 4.768 | 0.000 | 0.000 | 0.000 | 0.000 | 0.000 | 0.000 | 0.000 | 0.000 | 0.000 |
| THYMU2040925 | 0.000 | 0.000 | 0.000 | 45.981 | 0.000 | 0.000 | 0.000 | 0.000 | 0.000 | 0.000 | 0.000 | 0.000 | 0.000 |
| THYMU3000360 | 0.000 | 0.000 | 0.000 | 27.904 | 0.000 | 0.000 | 39.314 | 0.000 | 0.000 | 0.000 | 0.000 | 0.000 | 0.000 |
| TKIDN2018926 | 0.000 | 0.000 | 0.000 | 0.000 | 0.000 | 21.396 | 0.000 | 0.000 | 0.000 | 0.000 | 0.000 | 0.000 | 0.000 |
| TLIVE2002046 | 3.353 | 4.524 | 2.255 | 7.729 | 0.000 | 10.972 | 5.445 | 4.164 | 0.000 | 1.852 | 0.000 | 0.000 | 12.076 |
| TRACH3000134 | 0.000 | 0.000 | 0.000 | 0.000 | 0.000 | 0.000 | 36.165 | 0.000 | 0.000 | 0.000 | 0.000 | 0.000 | 0.000 |
| UTERU2008040 | 0.000 | 0.000 | 0.000 | 8.353 | 0.000 | 11.858 | 0.000 | 0.000 | 0.000 | 24.014 | 0.000 | 0.000 | 0.000 |
| UTERU2011220 | 0.000 | 0.000 | 0.000 | 1.389 | 0.000 | 0.000 | 0.000 | 0.000 | 0.000 | 0.000 | 0.000 | 0.000 | 0.000 |
| UTERU2021820 | 0.000 | 0.000 | 0.000 | 17.694 | 0.000 | 0.000 | 24.929 | 0.000 | 0.000 | 0.000 | 0.000 | 0.000 | 27.645 |
| UTERU2028734 | 0.000 | 0.000 | 27.272 | 0.000 | 0.000 | 0.000 | 21.953 | 0.000 | 0.000 | 0.000 | 0.000 | 0.000 | 0.000 |

The result of comparative analysis of cDNA libraries derived from fetal heart (FEHRT) and adult heart (HEART) showed the following genes whose expression levels differed between the two.

TABLE 22

| Clone ID | FEHRT | HEART |
|---|---|---|
| BRAMY2040592 | 0.000 | 23.231 |
| BRAWH2001671 | 0.000 | 25.367 |
| BRSTN2016470 | 0.000 | 1.460 |
| CTONG2017500 | 55.163 | 0.000 |
| CTONG2028124 | 0.000 | 3.353 |
| CTONG3000657 | 0.000 | 25.884 |
| D3OST3000169 | 10.191 | 0.000 |
| FEBRA2008287 | 61.381 | 0.000 |
| HCHON2000244 | 13.698 | 0.000 |
| HCHON2000626 | 0.000 | 7.842 |
| HEART1000010 | 0.000 | 22.793 |
| HEART1000074 | 0.000 | 5.064 |
| HEART1000088 | 0.000 | 49.759 |
| HEART1000139 | 0.000 | 66.452 |
| HEART2001680 | 0.000 | 100.000 |
| HEART2001756 | 0.000 | 100.000 |
| HEART2006131 | 0.000 | 100.000 |
| HEART2006909 | 0.000 | 100.000 |
| HEART2007031 | 0.000 | 75.806 |
| HEART2010391 | 0.000 | 100.000 |
| HEART2010492 | 0.000 | 100.000 |
| HEART2010495 | 0.000 | 49.759 |
| KIDNE2000665 | 0.000 | 12.914 |
| NB9N41000340 | 0.000 | 9.675 |
| NT2RI2003993 | 0.000 | 25.326 |
| NT2RI3002892 | 0.000 | 11.740 |
| OCBBF2024850 | 0.000 | 14.826 |
| SKMUS2006394 | 0.000 | 32.130 |
| SMINT2001818 | 0.000 | 6.254 |
| TESTI4000209 | 55.163 | 0.000 |
| TKIDN2015788 | 54.776 | 0.000 |
| TRACH3002192 | 12.987 | 0.000 |
| TRACH3005294 | 77.338 | 0.000 |
| TRACH3007479 | 0.000 | 7.175 |
| HEART2009680 | 100.000 | 0.000 |
| THYMU2004284 | 89.631 | 0.000 |

The result of comparative analysis of cDNA libraries derived from fetal kidney (FEKID) and adult kidney (KIDNE) showed the following genes whose expression levels differed between the two.

TABLE 23

| Clone ID | FEKID | KIDNE |
|---|---|---|
| BRACE2043665 | 0.000 | 83.085 |
| BRACE3010428 | 0.000 | 3.991 |
| BRSTN2016470 | 0.000 | 0.763 |
| CTONG1000087 | 0.000 | 3.646 |
| CTONG2028124 | 0.000 | 3.504 |
| CTONG3008894 | 0.000 | 2.534 |
| HCASM2003415 | 36.285 | 0.000 |
| HCHON2000244 | 0.000 | 4.587 |
| HEART1000074 | 0.000 | 2.646 |
| HHDPC1000118 | 0.000 | 7.307 |
| KIDNE1000064 | 0.000 | 100.000 |
| KIDNE2000665 | 41.875 | 13.498 |
| KIDNE2000722 | 0.000 | 100.000 |
| KIDNE2000832 | 67.408 | 32.592 |
| KIDNE2000846 | 0.000 | 100.000 |
| KIDNE2001361 | 0.000 | 80.699 |
| KIDNE2001847 | 0.000 | 12.687 |
| KIDNE2002252 | 0.000 | 7.600 |
| KIDNE2002991 | 0.000 | 100.000 |
| KIDNE2003837 | 0.000 | 100.000 |
| KIDNE2005543 | 0.000 | 62.084 |
| KIDNE2006580 | 0.000 | 48.900 |
| KIDNE2010264 | 0.000 | 100.000 |
| KIDNE2011314 | 0.000 | 75.867 |
| KIDNE2011532 | 0.000 | 100.000 |
| KIDNE2011635 | 0.000 | 48.502 |
| KIDNE2012945 | 0.000 | 48.732 |
| KIDNE2013095 | 0.000 | 100.000 |
| PEBLM2004666 | 0.000 | 12.953 |
| PLACE6019385 | 87.742 | 0.000 |
| STOMA1000189 | 0.000 | 16.356 |
| SYNOV4007671 | 0.000 | 2.556 |
| TBAES2001258 | 0.000 | 33.137 |
| TESOP2002451 | 51.382 | 0.000 |
| TESTI4000014 | 0.000 | 1.245 |
| TESTI4012702 | 0.000 | 7.600 |
| THYMU2032014 | 0.000 | 57.532 |
| TRACH2001684 | 0.000 | 62.100 |
| TRACH2007834 | 0.000 | 1.758 |
| UTERU2023175 | 0.000 | 5.190 |
| NT2RI2009583 | 10.920 | 4.840 |
| OCBBF2008144 | 0.000 | 62.383 |

The result of comparative analysis of cDNA libraries derived from fetal lung (FELNG) and adult lung (HLUNG) showed the following genes whose expression levels differed between the two.

TABLE 24

| Clone ID | FELNG | HLUNG |
|---|---|---|
| BRAWH3007592 | 38.566 | 0.000 |
| BRCAN2021028 | 0.000 | 38.589 |
| BRHIP2000819 | 0.000 | 7.923 |
| BRSTN2016470 | 4.707 | 0.803 |
| CTONG1000087 | 0.000 | 3.835 |
| CTONG2028124 | 10.809 | 9.217 |
| HCASM2007047 | 73.789 | 0.000 |
| HEART1000074 | 32.652 | 2.784 |
| HLUNG1000017 | 0.000 | 100.000 |
| HLUNG2000014 | 0.000 | 100.000 |
| HLUNG2001996 | 0.000 | 76.784 |
| HLUNG2002465 | 26.011 | 4.436 |
| HLUNG2002958 | 0.000 | 100.000 |
| HLUNG2003003 | 0.000 | 29.311 |
| HLUNG2003872 | 0.000 | 100.000 |
| HLUNG2010464 | 0.000 | 100.000 |
| HLUNG2011041 | 0.000 | 100.000 |
| HLUNG2011298 | 0.000 | 35.254 |
| HLUNG2012049 | 0.000 | 100.000 |
| HLUNG2012287 | 0.000 | 100.000 |
| HLUNG2012727 | 0.000 | 100.000 |
| HLUNG2013204 | 0.000 | 100.000 |
| HLUNG2013304 | 0.000 | 100.000 |
| HLUNG2013622 | 0.000 | 100.000 |
| HLUNG2013851 | 0.000 | 100.000 |
| HLUNG2014262 | 0.000 | 100.000 |
| HLUNG2014288 | 0.000 | 100.000 |
| HLUNG2014449 | 0.000 | 100.000 |
| HLUNG2015617 | 0.000 | 100.000 |
| HLUNG2017350 | 0.000 | 100.000 |
| HLUNG2017546 | 0.000 | 12.944 |
| HLUNG2017806 | 0.000 | 100.000 |
| HLUNG2019058 | 0.000 | 100.000 |
| HSYRA2008376 | 0.000 | 11.470 |
| KIDNE2012945 | 0.000 | 51.268 |
| NT2RI2003993 | 0.000 | 13.924 |
| NT2RI3007543 | 18.247 | 0.000 |
| OCBBF3000483 | 0.000 | 14.638 |
| SMINT1000192 | 64.044 | 0.000 |
| SPLEN2028914 | 0.000 | 10.242 |
| SPLEN2031547 | 0.000 | 5.775 |
| STOMA1000189 | 33.633 | 0.000 |
| SYNOV4007671 | 0.000 | 2.689 |
| TESOP1000127 | 0.000 | 34.899 |
| TESTI2003573 | 0.000 | 27.394 |
| TESTI4000014 | 0.000 | 0.655 |
| TESTI4037156 | 0.000 | 2.030 |
| TRACH2005811 | 0.000 | 3.868 |
| TRACH3004068 | 0.000 | 5.227 |
| UTERU2005621 | 0.000 | 8.268 |
| UTERU2023175 | 32.020 | 0.000 |
| FEBRA2028516 | 4.195 | 0.000 |
| HCHON2000508 | 1.370 | 0.000 |
| HLUNG2013350 | 100.000 | 0.000 |
| HLUNG2015418 | 76.605 | 0.000 |
| HLUNG2015548 | 100.000 | 0.000 |
| HLUNG2016862 | 100.000 | 0.000 |
| TESTI2015626 | 2.412 | 0.000 |
| TRACH2019672 | 76.605 | 0.000 |

### EXAMPLE 9

### In-Silico Expression Frequency Analysis Based on Large-Scale Analysis Data for 5′-End Sequences

Analyzing the expression level of a gene in various organs, tissues or cells is an exceedingly important step in clarifying the function of that organ, tissue, or cell, as well as in clarifying gene networks and in studying a causative gene of a morbid state. For example, if the expression level of a gene differs between a normal tissue and a malignant tissue, there is a possibility that that gene is involved in cancer. If tissue canceration can be suppressed by downregulating the expression of that gene, a compound comprising such downregulating activity can be used as an anticancer drug.

Various methods for analyzing gene expression frequency have been developed. For example, wet-type experiment-based methods include Northern blotting and RT-PCR, and of the use of gene chips and microarrays, where target samples synthesized from tissue or cell-derived RNA are hybridized to polynucleotides that comprise partial gene sequences synthesized on a base, or cDNA clones attached as plasmids directly to a base, and then signals are detected (Experiment Medicine, Vol. 17, No. 8, 980-1056 (1999), Eds., Muramatsu and Nawa, Cell Technology, Suppl. “DNA Microarray and New PCR Methods” (Shujunsha, 2000)). A method called “ATAC-PCR” is also available (Kato. K (1997) Nucleic Acids Res. 25, 4694-6), which comprises the steps of cleaving cDNA synthesized from tissue or cell-derived RNA, attaching adapters of different length depending on the type of tissue or cell, carrying out competitive PCR using a primer which contains a fluorescent dye and a sequence complementary to the adapter, and a primer specific to the gene, and then analyzing the expression level of the gene. In addition, an in-silico analysis-based method using sequence data is available. A database called BODYMAP (http://bodymap.ims.u-tokyo.ac.jp/) has been constructed by randomly extracting gene clones from cDNA libraries of various tissues and cells, combining clones homologous to one another as a cluster, classifying the genes in each cluster unit based on homology information on the nucleotide sequences of cDNA 3′ ends, and then obtaining information on gene expression frequency by comparing the number of clones in respective clusters.

Both experiment-based and in-silico analysis-based methods are widely accepted methods of gene expression analysis. These methods, and almost all other established methods for analyzing the frequency of gene expression, are based on analysis of 3′-end gene sequences. In human genes the 3′-untranslated regions (3′-UTR) are usually long, and thus there is controversy as to whether the results of such methods directly correspond to gene expression frequency and polypeptide expression pattern. However, the analysis method for gene expression frequency of the present invention is based on 5′-end gene sequences, and thus the expression pattern of each gene is a more accurate and faithful reproduction of the original in vivo pattern.

cDNA libraries in which the probability of cDNA completeness is exceedingly high were prepared from various tissues and cells as described in Example 1. cDNA clones were randomly selected from each library, the cDNA 5′-end sequences were determined, and a sequence database was constructed using this data. This database contains the nucleotide sequences of 1,402,069 clones, a sufficiently large population for analysis. The libraries used in the analysis, the number of cDNA clones whose 5′-end sequences were determined, and the weighted value for a single sequence in each library (100 divided by the number of sequences analyzed in each library) are described below.

TABLE 25

| | Number of clones | Weighted values |
|---|---|---|
| Library | whose 5′ ends | for single sequences |
| Names | were analyzed | (100/Number of sequences) |
| 3NB69 | 8182 | 0.012221951 |
| ACTVT | 684 | 0.14619883 |
| ADIPS | 614 | 0.16286645 |
| ADRGL | 10300 | 0.009708738 |
| AHMSC | 671 | 0.149031297 |
| ASTRO | 17226 | 0.005805178 |
| BEAST | 2736 | 0.036549708 |
| BGGI1 | 1904 | 0.052521008 |
| BLADE | 8503 | 0.011760555 |
| BNGH4 | 7739 | 0.012921566 |
| BRACE | 84087 | 0.001189244 |
| BRALZ | 16517 | 0.006054368 |
| BRAMY | 59232 | 0.001688277 |
| BRASW | 158 | 0.632911392 |
| BRAWH | 59682 | 0.001675547 |
| BRCAN | 26014 | 0.003844084 |
| BRCOC | 16847 | 0.005935775 |
| BRHIP | 58498 | 0.00170946 |
| BRSSN | 16035 | 0.006236358 |
| BRSTN | 16552 | 0.006041566 |
| BRTHA | 53818 | 0.001858114 |
| CD34C | 1421 | 0.070372977 |
| CERVX | 2888 | 0.034626039 |
| CHONS | 2694 | 0.037119525 |
| COLON | 8501 | 0.011763322 |
| CORDB | 711 | 0.140646976 |
| CTONG | 32043 | 0.003120806 |
| D3OST | 5112 | 0.019561815 |
| D6OST | 889 | 0.112485939 |
| D9OST | 4426 | 0.022593764 |
| DFNES | 10126 | 0.009874593 |
| ERLTF | 2178 | 0.045913682 |
| FCBBF | 32305 | 0.003095496 |
| FEBRA | 23941 | 0.004176935 |
| FEHRT | 2866 | 0.034891835 |
| FEKID | 2759 | 0.036245016 |
| FELIV | 186 | 0.537634409 |
| FELNG | 2775 | 0.036036036 |
| HCASM | 8989 | 0.011124708 |
| HCHON | 9432 | 0.010602205 |
| HEART | 8946 | 0.01117818 |
| HELAC | 680 | 0.147058824 |
| HHDPC | 8476 | 0.011798018 |
| HLUNG | 16272 | 0.006145526 |
| HSYRA | 7985 | 0.012523482 |
| IMR32 | 16914 | 0.005912262 |
| JCMLC | 2171 | 0.046061723 |
| KIDNE | 17119 | 0.005841463 |
| LIVER | 6885 | 0.014524328 |
| LYMPB | 2630 | 0.038022814 |
| MAMGL | 184 | 0.543478261 |
| MESAN | 16095 | 0.00621311 |
| MESTC | 691 | 0.1447178 |
| N1ESE | 2628 | 0.03805175 |
| NB9N4 | 1764 | 0.056689342 |
| NCRRM | 703 | 0.142247511 |
| NCRRP | 699 | 0.143061516 |
| NESOP | 2805 | 0.035650624 |
| NETRP | 9236 | 0.010827198 |
| NHNPC | 2392 | 0.04180602 |
| NOVAR | 2504 | 0.039936102 |
| NT2NE | 16468 | 0.006072383 |
| NT2RI | 32842 | 0.003044882 |
| NT2RM | 2074 | 0.048216008 |
| NT2RP | 24763 | 0.004038283 |
| NTISM | 181 | 0.552486188 |
| NTONG | 8098 | 0.012348728 |
| OCBBF | 48042 | 0.002081512 |
| PANCR | 182 | 0.549450549 |
| PEBLM | 7940 | 0.012594458 |
| PERIC | 8860 | 0.011286682 |
| PLACE | 33535 | 0.002981959 |
| PROST | 16829 | 0.005942124 |
| PUAEN | 10577 | 0.009454477 |
| RECTM | 2743 | 0.036456435 |
| SALGL | 185 | 0.540540541 |
| SKMUS | 8470 | 0.011806375 |
| SKNMC | 7656 | 0.013061651 |
| SKNSH | 8692 | 0.011504832 |
| SMINT | 16913 | 0.005912612 |
| SPLEN | 34307 | 0.002914857 |
| STOMA | 8770 | 0.011402509 |
| SYNOV | 27671 | 0.003613892 |
| T1ESE | 2687 | 0.037216226 |
| TBAES | 8484 | 0.011786893 |
| TCERX | 2828 | 0.035360679 |
| TCOLN | 2815 | 0.035523979 |
| TESOP | 8723 | 0.011463946 |
| TESTI | 91301 | 0.001095278 |
| THYMU | 71574 | 0.001397155 |
| TKIDN | 16123 | 0.00620232 |
| TLIVE | 8681 | 0.01151941 |
| TLUNG | 2884 | 0.034674064 |
| TOVAR | 2740 | 0.03649635 |
| TRACH | 53281 | 0.001876842 |
| TSTOM | 2779 | 0.035984167 |
| TUTER | 2678 | 0.037341299 |
| UMVEN | 633 | 0.157977883 |
| UTERU | 50040 | 0.00199 [symbol] 401 |

Using a nucleotide sequence homology search program, the nucleotide sequences of respective clones in this database were categorized (clustered) into groups, each of which contained homologous sequences. Cluster members (the number of clones whose 5′ ends were analyzed) were totalized and normalized for each library, resulting in an abundance ratio of genes in the cDNA library. In addition, 17,547 genes, including full-length cDNAs of the present invention, were assigned to a cluster using a nucleotide sequence homology search program.

In addition to the abundance ratio of the assigned genes in cDNA libraries, the sums of the weighted values for single sequences in each library were also calculated. For example, the expression of the clone BRACE2000753 was detectable in the BRACE, SPLEN, and THYMU libraries, based on expression analysis results. The single-sequence weighted values for these libraries are 0.001189244, 0.002914857, and 0.001397155 respectively, as seen in Table 1. Thus, their sum is 0.005501257. This parameter serves as an index for gene expression frequency in entire libraries. The higher this value, the greater the gene expression level. Listed below are the clone names of the 17,176 genes assigned to clusters, the number of cDNA clones whose 5′ end sequences were determined, and the sum of the weighted values for single sequences, cluster names, and the number of clones whose 5′ ends were analyzed.

LENGTHY TABLE REFERENCED HERE
US20070020637A1-20070125-T00001
Please refer to the end of the specification for access instructions.

This analysis yielded information on the expression frequencies of the 17,176 genes that include the cDNAs of the present invention, in all tissues and cells used as sources of the respective cDNA libraries and described in Example 1.

Of the 17,176 genes that include the cDNAs of the present invention, genes whose expression levels were high enough to detect in each of the 3NB69, ACTVT, ADIPS, ADRGL, AHMSC, ASTRO, and BEAST libraries were analyzed for expression frequency. The analysis results are described below. Each is a relative value, where the expression level over all tissues is taken as 100. The higher this value, the greater the level of expression.

| Clone Name | 3NB69 | ACTVT | ADIPS | ADRGL | AHMSC | ASTRO | BEAST |
|---|---|---|---|---|---|---|---|
| 3NB691000018 | 67.798 | 0 | 0 | 0 | 0 | 32.202 | 0 |
| 3NB692000281 | 1.606 | 0 | 0 | 0 | 19.579 | 0.763 | 0 |
| 3NB692000330 | 6.435 | 0 | 0 | 0 | 39.233 | 1.528 | 0 |
| 3NB692000374 | 15.821 | 0 | 0 | 0 | 0 | 3.757 | 0 |
| 3NB692000912 | 3.427 | 0 | 0 | 5.445 | 0 | 0 | 0 |
| 3NB692001022 | 18.151 | 0 | 0 | 0 | 0 | 4.311 | 0 |
| 3NB692001538 | 28.982 | 0 | 0 | 11.511 | 0 | 0 | 0 |
| ACTVT2000380 | 0 | 100 | 0 | 0 | 0 | 0 | 0 |
| ADIPS1000064 | 0 | 0 | 100 | 0 | 0 | 0 | 0 |
| ADIPS2000069 | 0 | 0 | 100 | 0 | 0 | 0 | 0 |
| ADIPS2000088 | 0 | 0 | 100 | 0 | 0 | 0 | 0 |
| ADIPS2000245 | 0 | 0 | 100 | 0 | 0 | 0 | 0 |
| ADIPS2000425 | 0 | 0 | 75.455 | 0 | 0 | 0 | 0 |
| ADRGL1000002 | 0 | 0 | 0 | 9.541 | 0 | 0 | 0 |
| ADRGL1000018 | 0 | 0 | 0 | 28.021 | 0 | 0 | 0 |
| ADRGL1000033 | 27.851 | 0 | 0 | 22.124 | 0 | 0 | 0 |
| ADRGL1000038 | 0 | 0 | 0 | 100 | 0 | 0 | 0 |
| ADRGL1000065 | 0 | 0 | 0 | 32.676 | 0 | 0 | 0 |
| ADRGL1000067 | 0 | 0 | 0 | 100 | 0 | 0 | 0 |
| ADRGL1000111 | 0 | 0 | 0 | 87.420 | 0 | 0 | 0 |
| ADRGL1000144 | 0 | 0 | 0 | 19.366 | 0 | 0 | 0 |
| ADRGL1000147 | 0 | 0 | 0 | 100 | 0 | 0 | 0 |
| ADRGL1000160 | 0 | 0 | 0 | 100 | 0 | 0 | 0 |
| ADRGL1000165 | 0 | 0 | 0 | 100 | 0 | 0 | 0 |
| ADRGL1000182 | 0 | 0 | 0 | 100 | 0 | 0 | 0 |
| ADRGL2000006 | 0 | 0 | 0 | 6.024 | 0 | 3.602 | 0 |
| ADRGL2000042 | 0 | 0 | 0 | 29.430 | 0 | 0 | 0 |
| ADRGL2000056 | 0 | 0 | 0 | 87.420 | 0 | 0 | 0 |
| ADRGL2000064 | 0 | 0 | 0 | 100 | 0 | 0 | 0 |
| ADRGL2000074 | 0 | 0 | 0 | 85.187 | 0 | 0 | 0 |
| ADRGL2000085 | 0 | 0 | 0 | 100 | 0 | 0 | 0 |
| ADRGL2000097 | 0 | 0 | 0 | 12.882 | 0 | 0 | 0 |
| ADRGL2000117 | 0 | 0 | 0 | 49.455 | 0 | 0 | 0 |
| ADRGL2000142 | 0 | 0 | 0 | 100 | 0 | 0 | 0 |
| ADRGL2000172 | 0 | 0 | 0 | 100 | 0 | 0 | 0 |
| ADRGL2000248 | 0 | 0 | 0 | 100 | 0 | 0 | 0 |
| ADRGL2000261 | 0 | 0 | 0 | 100 | 0 | 0 | 0 |
| ADRGL2000323 | 0 | 0 | 0 | 76.909 | 0 | 0 | 0 |
| ADRGL2000328 | 0 | 0 | 0 | 78.014 | 0 | 0 | 0 |
| ADRGL2000353 | 0 | 0 | 0 | 100 | 0 | 0 | 0 |
| ADRGL2000384 | 0 | 0 | 0 | 100 | 0 | 0 | 0 |
| ADRGL2000428 | 0 | 0 | 0 | 100 | 0 | 0 | 0 |
| ADRGL2000636 | 0 | 0 | 0 | 100 | 0 | 0 | 0 |
| ADRGL2000644 | 0 | 0 | 0 | 61.592 | 0 | 0 | 0 |
| ADRGL2000968 | 0 | 0 | 0 | 100 | 0 | 0 | 0 |
| ADRGL2001119 | 0 | 0 | 0 | 100 | 0 | 0 | 0 |
| ADRGL2001229 | 0 | 0 | 0 | 100 | 0 | 0 | 0 |
| ADRGL2001287 | 0 | 0 | 0 | 100 | 0 | 0 | 0 |
| ADRGL2001301 | 0 | 0 | 0 | 100 | 0 | 0 | 0 |
| ADRGL2001354 | 0 | 0 | 0 | 100 | 0 | 0 | 0 |
| ADRGL2001554 | 0 | 0 | 0 | 100 | 0 | 0 | 0 |
| ADRGL2001651 | 0 | 0 | 0 | 100 | 0 | 0 | 0 |
| ADRGL2001756 | 0 | 0 | 0 | 100 | 0 | 0 | 0 |
| ADRGL2001830 | 0 | 0 | 0 | 69.255 | 0 | 0 | 0 |
| ADRGL2001836 | 0 | 0 | 0 | 100 | 0 | 0 | 0 |
| ADRGL2001854 | 0 | 0 | 0 | 100 | 0 | 0 | 0 |
| ADRGL2002013 | 0 | 0 | 0 | 52.125 | 0 | 0 | 0 |
| ADRGL2002029 | 0 | 0 | 0 | 100 | 0 | 0 | 0 |
| ADRGL2002191 | 0 | 0 | 0 | 74.076 | 0 | 0 | 0 |
| ADRGL2002260 | 0 | 0 | 0 | 87.420 | 0 | 0 | 0 |
| ADRGL2002392 | 0 | 0 | 0 | 100 | 0 | 0 | 0 |
| ADRGL2002477 | 0 | 0 | 0 | 9.857 | 0 | 0 | 0 |
| ADRGL2002679 | 0 | 0 | 0 | 72.099 | 0 | 0 | 0 |
| ADRGL2002753 | 0 | 0 | 0 | 100 | 0 | 0 | 0 |
| ADRGL2002857 | 0 | 0 | 0 | 100 | 0 | 0 | 0 |
| ADRGL2003017 | 0 | 0 | 0 | 100 | 0 | 0 | 0 |
| ADRGL2003329 | 0 | 0 | 0 | 100 | 0 | 0 | 0 |
| ADRGL2003552 | 0 | 0 | 0 | 100 | 0 | 0 | 0 |
| ADRGL2003585 | 0 | 0 | 0 | 100 | 0 | 0 | 0 |
| ADRGL2003638 | 0 | 0 | 0 | 100 | 0 | 0 | 0 |
| ADRGL2003684 | 0 | 0 | 0 | 100 | 0 | 0 | 0 |
| ADRGL2003773 | 0 | 0 | 0 | 100 | 0 | 0 | 0 |
| ADRGL2003785 | 0 | 0 | 0 | 85.029 | 0 | 0 | 0 |
| ADRGL2004031 | 0 | 0 | 0 | 100 | 0 | 0 | 0 |
| ADRGL2004077 | 0 | 0 | 0 | 100 | 0 | 0 | 0 |
| ADRGL2004451 | 0 | 0 | 0 | 100 | 0 | 0 | 0 |
| ADRGL2004459 | 0 | 0 | 0 | 100 | 0 | 0 | 0 |
| ADRGL2004676 | 0 | 0 | 0 | 50.515 | 0 | 0 | 0 |
| ADRGL2004708 | 0 | 0 | 0 | 100 | 0 | 0 | 0 |
| ADRGL2004724 | 0 | 0 | 0 | 100 | 0 | 0 | 0 |
| ADRGL2004749 | 0 | 0 | 0 | 100 | 0 | 0 | 0 |
| ADRGL2004770 | 0 | 0 | 0 | 100 | 0 | 0 | 0 |
| ADRGL2004777 | 0 | 0 | 0 | 100 | 0 | 0 | 0 |
| ADRGL2004833 | 0 | 0 | 0 | 100 | 0 | 0 | 0 |
| ADRGL2005336 | 0 | 0 | 0 | 100 | 0 | 0 | 0 |
| ADRGL2005564 | 0 | 0 | 0 | 100 | 0 | 0 | 0 |
| ADRGL2005756 | 0 | 0 | 0 | 100 | 0 | 0 | 0 |
| ADRGL2005838 | 0 | 0 | 0 | 61.237 | 0 | 0 | 0 |
| ADRGL2005926 | 0 | 0 | 0 | 100 | 0 | 0 | 0 |
| ADRGL2005961 | 0 | 0 | 0 | 100 | 0 | 0 | 0 |
| ADRGL2006034 | 0 | 0 | 0 | 100 | 0 | 0 | 0 |
| ADRGL2006124 | 0 | 0 | 0 | 100 | 0 | 0 | 0 |
| ADRGL2006177 | 0 | 0 | 0 | 100 | 0 | 0 | 0 |
| ADRGL2006193 | 0 | 0 | 0 | 100 | 0 | 0 | 0 |
| ADRGL2006377 | 0 | 0 | 0 | 100 | 0 | 0 | 0 |
| ADRGL2006677 | 0 | 0 | 0 | 100 | 0 | 0 | 0 |
| ADRGL2006732 | 0 | 0 | 0 | 100 | 0 | 0 | 0 |
| ADRGL2006767 | 0 | 0 | 0 | 100 | 0 | 0 | 0 |
| ADRGL2006817 | 0 | 0 | 0 | 100 | 0 | 0 | 0 |
| ADRGL2006846 | 0 | 0 | 0 | 100 | 0 | 0 | 0 |
| ADRGL2006886 | 0 | 0 | 0 | 100 | 0 | 0 | 0 |
| ADRGL2007080 | 0 | 0 | 0 | 100 | 0 | 0 | 0 |
| ADRGL2007125 | 0 | 0 | 0 | 100 | 0 | 0 | 0 |
| ADRGL2007283 | 0 | 0 | 0 | 100 | 0 | 0 | 0 |
| ADRGL2007313 | 0 | 0 | 0 | 56.775 | 0 | 0 | 0 |
| ADRGL2007357 | 0 | 0 | 0 | 100 | 0 | 0 | 0 |
| ADRGL2007636 | 0 | 0 | 0 | 100 | 0 | 0 | 0 |
| ADRGL2007651 | 0 | 0 | 0 | 100 | 0 | 0 | 0 |
| ADRGL2007810 | 0 | 0 | 0 | 100 | 0 | 0 | 0 |
| ADRGL2007877 | 0 | 0 | 0 | 100 | 0 | 0 | 0 |
| ADRGL2007906 | 0 | 0 | 0 | 52.219 | 0 | 0 | 0 |
| ADRGL2007974 | 0 | 0 | 0 | 85.187 | 0 | 0 | 0 |
| ADRGL2008023 | 0 | 0 | 0 | 100 | 0 | 0 | 0 |
| ADRGL2008331 | 0 | 0 | 0 | 100 | 0 | 0 | 0 |
| ADRGL2008337 | 0 | 0 | 0 | 100 | 0 | 0 | 0 |
| ADRGL2008535 | 0 | 0 | 0 | 87.420 | 0 | 0 | 0 |
| ADRGL2008966 | 0 | 0 | 0 | 85.187 | 0 | 0 | 0 |
| ADRGL2009146 | 0 | 0 | 0 | 89.087 | 0 | 0 | 0 |
| ADRGL2009273 | 0 | 0 | 0 | 8.049 | 0 | 0 | 0 |
| ADRGL2009324 | 0 | 0 | 0 | 100 | 0 | 0 | 0 |
| ADRGL2009489 | 0 | 0 | 0 | 100 | 0 | 0 | 0 |
| ADRGL2009533 | 0 | 0 | 0 | 100 | 0 | 0 | 0 |
| ADRGL2009691 | 0 | 0 | 0 | 100 | 0 | 0 | 0 |
| ADRGL2009755 | 0 | 0 | 0 | 100 | 0 | 0 | 0 |
| ADRGL2010152 | 0 | 0 | 0 | 33.564 | 0 | 0 | 0 |
| ADRGL2010315 | 0 | 0 | 0 | 100 | 0 | 0 | 0 |
| ADRGL2010594 | 0 | 0 | 0 | 100 | 0 | 0 | 0 |
| ADRGL2010860 | 0 | 0 | 0 | 100 | 0 | 0 | 0 |
| ADRGL2010974 | 0 | 0 | 0 | 100 | 0 | 0 | 0 |
| ADRGL2011190 | 0 | 0 | 0 | 100 | 0 | 0 | 0 |
| ADRGL2011330 | 0 | 0 | 0 | 87.420 | 0 | 0 | 0 |
| ADRGL2012038 | 0 | 0 | 0 | 11.911 | 0 | 0 | 0 |
| ADRGL2012179 | 0 | 0 | 0 | 82.345 | 0 | 0 | 0 |
| AHMSC1000138 | 0 | 0 | 0 | 0 | 100 | 0 | 0 |
| ASTRO1000009 | 0 | 0 | 0 | 0 | 0 | 9.047 | 0 |
| ASTRO1000018 | 0 | 0 | 0 | 0 | 0 | 100 | 0 |
| ASTRO1000029 | 0 | 0 | 0 | 0 | 0 | 100 | 0 |
| ASTRO1000096 | 0 | 0 | 0 | 0 | 0 | 34.903 | 0 |
| ASTRO1000165 | 0 | 0 | 0 | 0 | 0 | 7.679 | 0 |
| ASTRO2000011 | 0 | 0 | 0 | 0 | 0 | 100 | 0 |
| ASTRO2000014 | 0 | 0 | 0 | 0 | 0 | 1.423 | 0 |
| ASTRO2000046 | 0 | 0 | 0 | 0 | 0 | 12.724 | 0 |
| ASTRO2000095 | 0 | 0 | 0 | 0 | 0 | 100 | 0 |
| ASTRO2000141 | 0 | 0 | 0 | 0 | 0 | 5.262 | 0 |
| ASTRO2000191 | 0 | 0 | 0 | 0 | 0 | 100 | 0 |
| ASTRO2000278 | 0 | 0 | 0 | 0 | 0 | 67.506 | 0 |
| ASTRO2000372 | 0 | 0 | 0 | 0 | 0 | 77.252 | 0 |
| ASTRO2000381 | 0 | 0 | 0 | 0 | 0 | 21.758 | 0 |
| ASTRO2000403 | 0 | 0 | 0 | 0 | 0 | 100 | 0 |
| ASTRO2000417 | 0 | 0 | 0 | 0 | 0 | 100 | 0 |
| ASTRO2000480 | 0 | 0 | 0 | 0 | 0 | 9.809 | 0 |
| ASTRO2000482 | 0 | 0 | 0 | 0 | 0 | 77.602 | 0 |
| ASTRO2000533 | 0 | 0 | 0 | 0 | 0 | 100 | 0 |
| ASTRO2000653 | 0 | 0 | 0 | 0 | 0 | 100 | 0 |
| ASTRO2000662 | 0 | 0 | 0 | 0 | 0 | 27.944 | 0 |
| ASTRO2000725 | 0 | 0 | 0 | 0 | 0 | 100 | 0 |
| ASTRO2000801 | 0 | 0 | 0 | 0 | 0 | 3.534 | 0 |
| ASTRO2000914 | 0 | 0 | 0 | 0 | 0 | 6.744 | 0 |
| ASTRO2001001 | 0 | 0 | 0 | 0 | 0 | 100 | 0 |
| ASTRO2001008 | 0 | 0 | 0 | 0 | 0 | 100 | 0 |
| ASTRO2001029 | 0 | 0 | 0 | 0 | 0 | 4.345 | 0 |
| ASTRO2001076 | 0 | 0 | 0 | 0 | 0 | 4.924 | 0 |
| ASTRO2001088 | 0 | 0 | 0 | 0 | 0 | 100 | 0 |
| ASTRO2001107 | 0 | 0 | 0 | 0 | 0 | 25.307 | 0 |
| ASTRO2001122 | 0 | 0 | 0 | 0 | 0 | 100 | 0 |
| ASTRO2001227 | 0 | 0 | 0 | 0 | 0 | 100 | 0 |
| ASTRO2001249 | 0 | 0 | 0 | 0 | 0 | 32.962 | 0 |
| ASTRO2001480 | 0 | 0 | 0 | 0 | 0 | 100 | 0 |
| ASTRO2001806 | 0 | 0 | 0 | 0 | 0 | 19.827 | 0 |
| ASTRO2001823 | 0 | 0 | 0 | 0 | 0 | 5.029 | 0 |
| ASTRO2002024 | 0 | 0 | 0 | 0 | 0 | 45.423 | 0 |
| ASTRO2002035 | 0 | 0 | 0 | 0 | 0 | 37.605 | 0 |
| ASTRO2002064 | 0 | 0 | 0 | 0 | 0 | 48.346 | 0 |
| ASTRO2002202 | 0 | 0 | 0 | 0 | 0 | 23.952 | 0 |
| ASTRO2002459 | 0 | 0 | 0 | 5.072 | 77.860 | 3.033 | 0 |
| ASTRO2002571 | 0 | 0 | 0 | 0 | 0 | 100 | 0 |
| ASTRO2002632 | 0 | 0 | 0 | 0 | 0 | 100 | 0 |
| ASTRO2002659 | 0 | 0 | 0 | 0 | 0 | 100 | 0 |
| ASTRO2002693 | 0 | 0 | 0 | 0 | 0 | 24.559 | 0 |
| ASTRO2002720 | 0 | 0 | 0 | 0 | 0 | 84.127 | 0 |
| ASTRO2002733 | 0 | 0 | 0 | 0 | 0 | 53.302 | 0 |
| ASTRO2002743 | 0 | 0 | 0 | 0 | 0 | 100 | 0 |
| ASTRO2002842 | 0 | 0 | 0 | 0 | 0 | 32.999 | 0 |
| ASTRO2003024 | 0 | 0 | 0 | 0 | 0 | 6.568 | 0 |
| ASTRO2003212 | 0 | 0 | 0 | 0 | 0 | 51.901 | 0 |
| ASTRO2003316 | 0 | 0 | 0 | 0 | 0 | 30.661 | 0 |
| ASTRO2003461 | 0 | 0 | 0 | 0 | 0 | 41.276 | 0 |
| ASTRO2003574 | 0 | 0 | 0 | 0 | 0 | 51.689 | 0 |
| ASTRO2003581 | 0 | 0 | 0 | 0 | 0 | 100 | 0 |
| ASTRO2003632 | 0 | 0 | 0 | 2.407 | 0 | 8.636 | 0 |
| ASTRO2003740 | 0 | 0 | 0 | 0 | 0 | 60.162 | 0 |
| ASTRO2003840 | 0 | 0 | 0 | 0 | 0 | 21.461 | 0 |
| ASTRO2003925 | 0 | 0 | 0 | 0 | 0 | 74.391 | 0 |
| ASTRO2003960 | 0 | 0 | 0 | 0 | 0 | 82.997 | 0 |
| ASTRO2004032 | 0 | 0 | 0 | 0 | 0 | 21.115 | 0 |
| ASTRO2004051 | 0 | 0 | 0 | 0 | 0 | 100 | 0 |
| ASTRO2004177 | 0 | 0 | 0 | 0 | 0 | 82.997 | 0 |
| ASTRO2004217 | 0 | 0 | 0 | 0 | 0 | 100 | 0 |
| ASTRO2004297 | 0 | 0 | 0 | 0 | 0 | 100 | 0 |
| ASTRO2004584 | 0 | 0 | 0 | 0 | 0 | 100 | 0 |
| ASTRO2004628 | 0 | 0 | 0 | 0 | 0 | 30.838 | 0 |
| ASTRO2004751 | 0 | 0 | 0 | 0 | 0 | 100 | 0 |
| ASTRO2004877 | 0 | 0 | 0 | 0 | 0 | 100 | 0 |
| ASTRO2004974 | 0 | 0 | 0 | 0 | 0 | 6.732 | 0 |
| ASTRO2005008 | 0 | 0 | 0 | 0 | 0 | 100 | 0 |
| ASTRO2005081 | 0 | 0 | 0 | 0 | 0 | 100 | 0 |
| ASTRO2005096 | 0 | 0 | 0 | 0 | 0 | 100 | 0 |
| ASTRO2005242 | 0 | 0 | 0 | 0 | 0 | 31.985 | 0 |
| ASTRO2005273 | 0 | 0 | 0 | 0 | 0 | 100 | 0 |
| ASTRO2005343 | 0 | 0 | 0 | 0 | 0 | 100 | 0 |
| ASTRO2005360 | 0 | 0 | 0 | 0 | 0 | 20.669 | 0 |
| ASTRO2005413 | 0 | 0 | 0 | 0 | 0 | 100 | 0 |
| ASTRO2005423 | 0 | 0 | 0 | 0 | 0 | 11.930 | 0 |
| ASTRO2005553 | 0 | 0 | 0 | 0 | 0 | 8.978 | 0 |
| ASTRO2005557 | 0 | 0 | 0 | 0 | 0 | 100 | 0 |
| ASTRO2005575 | 0 | 0 | 0 | 0 | 0 | 100 | 0 |
| ASTRO2005593 | 0 | 0 | 0 | 0 | 0 | 48.346 | 0 |
| ASTRO2005687 | 0 | 0 | 0 | 0 | 0 | 11.585 | 0 |
| ASTRO2005726 | 0 | 0 | 0 | 0 | 0 | 100 | 0 |
| ASTRO2005863 | 0 | 0 | 0 | 0 | 0 | 26.342 | 0 |
| ASTRO2005896 | 0 | 0 | 0 | 0 | 0 | 30.769 | 0 |
| ASTRO2005902 | 0 | 0 | 0 | 0 | 0 | 100 | 0 |
| ASTRO2006356 | 0 | 0 | 0 | 0 | 0 | 100 | 0 |
| ASTRO2006475 | 0 | 0 | 0 | 0 | 0 | 25.805 | 0 |
| ASTRO2006732 | 0 | 0 | 0 | 0 | 0 | 53.140 | 0 |
| ASTRO2006747 | 0 | 0 | 0 | 0 | 0 | 100 | 0 |
| ASTRO2006920 | 0 | 0 | 0 | 0 | 0 | 20.786 | 0 |
| ASTRO2006952 | 0 | 0 | 0 | 0 | 0 | 100 | 0 |
| ASTRO2007047 | 0 | 0 | 0 | 0 | 0 | 7.241 | 0 |
| ASTRO2007221 | 0 | 0 | 0 | 0 | 0 | 65.037 | 0 |
| ASTRO2007377 | 0 | 0 | 0 | 0 | 0 | 100 | 0 |
| ASTRO2007515 | 0 | 0 | 0 | 0 | 0 | 100 | 0 |
| ASTRO2007578 | 0 | 0 | 0 | 0 | 0 | 100 | 0 |
| ASTRO2007643 | 0 | 0 | 0 | 0 | 0 | 100 | 0 |
| ASTRO2007666 | 0 | 0 | 0 | 0 | 0 | 100 | 0 |
| ASTRO2007948 | 0 | 0 | 0 | 0 | 0 | 8.677 | 0 |
| ASTRO2007956 | 0 | 0 | 0 | 0 | 0 | 100 | 0 |
| ASTRO2007962 | 0 | 0 | 0 | 0 | 0 | 100 | 0 |
| ASTRO2008040 | 0 | 0 | 0 | 0 | 0 | 41.505 | 0 |
| ASTRO2008216 | 0 | 0 | 0 | 0 | 0 | 11.056 | 0 |
| ASTRO2008239 | 0 | 0 | 0 | 0 | 0 | 100 | 0 |
| ASTRO2008409 | 0 | 0 | 0 | 0 | 0 | 45.042 | 0 |
| ASTRO2008425 | 0 | 0 | 0 | 0 | 0 | 100 | 0 |
| ASTRO2008508 | 0 | 0 | 0 | 0 | 0 | 31.159 | 0 |
| ASTRO2008777 | 0 | 0 | 0 | 0 | 0 | 100 | 0 |
| ASTRO2008895 | 0 | 0 | 0 | 0 | 0 | 100 | 0 |
| ASTRO2008960 | 0 | 0 | 0 | 0 | 0 | 100 | 0 |
| ASTRO2008972 | 0 | 0 | 0 | 0 | 0 | 53.958 | 0 |
| ASTRO2009068 | 0 | 0 | 0 | 0 | 0 | 19.605 | 0 |
| ASTRO2009118 | 0 | 0 | 0 | 0 | 0 | 54.118 | 0 |
| ASTRO2009177 | 0 | 0 | 0 | 0 | 0 | 100 | 0 |
| ASTRO2009333 | 0 | 0 | 0 | 0 | 0 | 100 | 0 |
| ASTRO2009458 | 0 | 0 | 0 | 0 | 0 | 100 | 0 |
| ASTRO2009526 | 0 | 0 | 0 | 0 | 0 | 100 | 0 |
| ASTRO2009879 | 0 | 0 | 0 | 0 | 0 | 100 | 0 |
| ASTRO2010072 | 0 | 0 | 0 | 0 | 0 | 60.162 | 0 |
| ASTRO2010582 | 0 | 0 | 0 | 0 | 0 | 59.968 | 0 |
| ASTRO2010615 | 0 | 0 | 0 | 0 | 0 | 100 | 0 |
| ASTRO2010799 | 0 | 0 | 0 | 0 | 0 | 49.444 | 0 |
| ASTRO2010819 | 0 | 0 | 0 | 3.899 | 0 | 2.331 | 0 |
| ASTRO2010962 | 0 | 0 | 0 | 0 | 0 | 100 | 0 |
| ASTRO2010996 | 0 | 0 | 0 | 0 | 0 | 65.595 | 0 |
| ASTRO2011149 | 0 | 0 | 0 | 0 | 0 | 100 | 0 |
| ASTRO2011422 | 0 | 0 | 0 | 0 | 0 | 25.135 | 0 |
| ASTRO2011426 | 0 | 0 | 0 | 0 | 0 | 100 | 0 |
| ASTRO2011437 | 0 | 0 | 0 | 0 | 0 | 100 | 0 |
| ASTRO2011461 | 0 | 0 | 0 | 0 | 0 | 100 | 0 |
| ASTRO2011834 | 0 | 0 | 0 | 0 | 0 | 100 | 0 |
| ASTRO2011893 | 0 | 0 | 0 | 0 | 0 | 100 | 0 |
| ASTRO2012552 | 0 | 0 | 0 | 0 | 0 | 10.242 | 0 |
| ASTRO2013124 | 0 | 0 | 0 | 0 | 0 | 100 | 0 |
| ASTRO2013585 | 7.112 | 0 | 0 | 0 | 0 | 1.689 | 0 |
| ASTRO2013671 | 0 | 0 | 0 | 0 | 0 | 80.601 | 0 |
| ASTRO2013802 | 0 | 0 | 0 | 0 | 0 | 100 | 0 |
| ASTRO2014135 | 0 | 0 | 0 | 0 | 0 | 12.035 | 0 |
| ASTRO2014174 | 0 | 0 | 0 | 0 | 0 | 77.252 | 0 |
| ASTRO2014211 | 0 | 0 | 0 | 0 | 0 | 100 | 0 |
| ASTRO2014363 | 0 | 0 | 0 | 0 | 0 | 100 | 0 |
| ASTRO2014392 | 0 | 0 | 0 | 0 | 0 | 51.203 | 0 |
| ASTRO2014498 | 0 | 0 | 0 | 0 | 0 | 61.936 | 0 |
| ASTRO2014561 | 0 | 0 | 0 | 0 | 0 | 30.533 | 0 |
| ASTRO2014576 | 0 | 0 | 0 | 0 | 0 | 39.158 | 0 |
| ASTRO2014863 | 0 | 0 | 0 | 0 | 0 | 11.705 | 0 |
| ASTRO2014923 | 0 | 0 | 0 | 0 | 0 | 100 | 0 |
| ASTRO2014961 | 0 | 0 | 0 | 0 | 0 | 100 | 0 |
| ASTRO2015162 | 0 | 0 | 0 | 0 | 0 | 100 | 0 |
| ASTRO2015185 | 0 | 0 | 0 | 0 | 0 | 100 | 0 |
| ASTRO2015328 | 0 | 0 | 0 | 0 | 0 | 39.556 | 0 |
| ASTRO2015529 | 0 | 0 | 0 | 0 | 0 | 6.419 | 0 |
| ASTRO2015987 | 0 | 0 | 0 | 0 | 0 | 9.099 | 0 |
| ASTRO2016044 | 0 | 0 | 0 | 0 | 0 | 100 | 0 |
| ASTRO2016114 | 0 | 0 | 0 | 0 | 0 | 50.227 | 0 |
| ASTRO2016148 | 0 | 0 | 0 | 0 | 0 | 73.607 | 0 |
| ASTRO2016491 | 0 | 0 | 0 | 2.918 | 0 | 1.745 | 0 |
| ASTRO2016681 | 0 | 0 | 0 | 0 | 0 | 100 | 0 |
| ASTRO2016819 | 0 | 0 | 0 | 0 | 0 | 22.357 | 0 |
| ASTRO2016847 | 0 | 0 | 0 | 0 | 0 | 100 | 0 |
| ASTRO2016892 | 0 | 0 | 0 | 0 | 0 | 100 | 0 |
| ASTRO2017348 | 0 | 0 | 0 | 0 | 0 | 100 | 0 |
| ASTRO2017627 | 0 | 0 | 0 | 0 | 0 | 100 | 0 |
| ASTRO2018169 | 0 | 0 | 0 | 0 | 0 | 3.272 | 0 |
| ASTRO2018373 | 0 | 0 | 0 | 0 | 0 | 48.346 | 0 |
| ASTRO2018540 | 0 | 0 | 0 | 0 | 0 | 5.388 | 0 |
| ASTRO2019039 | 0 | 0 | 0 | 0 | 0 | 100 | 0 |
| ASTRO2019171 | 0 | 0 | 0 | 0 | 0 | 61.936 | 0 |
| ASTRO3000154 | 0 | 0 | 0 | 0 | 0 | 100 | 0 |
| ASTRO3000172 | 0 | 0 | 0 | 0 | 0 | 100 | 0 |
| ASTRO3000177 | 0 | 0 | 0 | 0 | 0 | 100 | 0 |
| ASTRO3000301 | 0 | 0 | 0 | 0 | 0 | 100 | 0 |
| ASTRO3000474 | 0 | 0 | 0 | 0 | 0 | 100 | 0 |
| ASTRO3000482 | 0 | 0 | 0 | 0 | 0 | 75.753 | 0 |
| BEAST2000454 | 0 | 0 | 0 | 0 | 0 | 0 | 100 |
| BGGI12000054 | 0 | 0 | 0 | 2.729 | 0 | 0.816 | 5.137 |
| BGGI12000616 | 0 | 0 | 0 | 5.181 | 0 | 0 | 0 |
| BGGI12001097 | 0 | 0 | 0 | 6.512 | 0 | 0 | 0 |
| BLADE2006830 | 0 | 0 | 0 | 0 | 0 | 5.824 | 0 |
| BNGH41000190 | 0 | 0 | 0 | 0 | 0 | 8.767 | 0 |
| BNGH41000198 | 0 | 0 | 0 | 24.076 | 0 | 0 | 0 |
| BNGH42000360 | 1.147 | 0 | 0 | 0.911 | 0 | 0 | 10.293 |
| BNGH42007460 | 0 | 0 | 0 | 5.839 | 0 | 0 | 0 |
| BNGH42007709 | 1.679 | 0 | 0 | 0 | 0 | 0.797 | 0 |
| BNGH42007788 | 0 | 0 | 0 | 0 | 0 | 10.666 | 0 |
| BRACE1000132 | 0 | 0 | 0 | 0 | 0 | 49.767 | 0 |
| BRACE2000148 | 0 | 0 | 0 | 20.686 | 0 | 0 | 0 |
| BRACE2000307 | 0 | 0 | 0 | 0 | 0 | 4.081 | 0 |
| BRACE2001143 | 0 | 0 | 0 | 8.607 | 0 | 0 | 0 |
| BRACE2001151 | 0 | 0 | 0 | 37.402 | 0 | 0 | 0 |
| BRACE2001692 | 0 | 0 | 58.856 | 0 | 0 | 0 | 0 |
| BRACE2002392 | 0 | 0 | 0 | 0 | 0 | 5.972 | 0 |
| BRACE2002441 | 0 | 0 | 0 | 0 | 0 | 11.706 | 0 |
| BRACE2002752 | 0 | 0 | 0 | 89.087 | 0 | 0 | 0 |
| BRACE2002860 | 0 | 0 | 0 | 14.588 | 0 | 0 | 0 |
| BRACE2003217 | 0 | 0 | 0 | 0 | 0 | 14.934 | 0 |
| BRACE2003254 | 0 | 0 | 0 | 0 | 0 | 3.137 | 0 |
| BRACE2003628 | 0 | 0 | 0 | 39.531 | 0 | 0 | 0 |
| BRACE2003785 | 0 | 0 | 0 | 0 | 0 | 3.367 | 0 |
| BRACE2003827 | 0 | 0 | 0 | 0 | 0 | 3.035 | 0 |
| BRACE2005151 | 0 | 0 | 0 | 12.175 | 0 | 0 | 0 |
| BRACE2005434 | 5.029 | 60.155 | 0 | 3.995 | 0 | 0 | 0 |
| BRACE2006537 | 0 | 0 | 0 | 6.236 | 0 | 0 | 0 |
| BRACE2006871 | 0 | 87.244 | 0 | 0 | 0 | 0 | 0 |
| BRACE2007089 | 0 | 0 | 0 | 4.906 | 0 | 0 | 0 |
| BRACE2007191 | 0 | 0 | 0 | 0 | 61.094 | 0 | 0 |
| BRACE2007671 | 0 | 0 | 0 | 0 | 0 | 5.357 | 0 |
| BRACE2007708 | 0 | 0 | 0 | 0 | 0 | 7.362 | 0 |
| BRACE2008097 | 0 | 0 | 0 | 27.819 | 0 | 0 | 0 |
| BRACE2008399 | 0 | 0 | 0 | 32.070 | 0 | 0 | 0 |
| BRACE2008402 | 0 | 0 | 0 | 24.298 | 0 | 0 | 0 |
| BRACE2008653 | 0 | 0 | 0 | 3.056 | 0 | 0 | 0 |
| BRACE2008706 | 0 | 0 | 0 | 19.039 | 0 | 0 | 0 |
| BRACE2008881 | 0 | 0 | 0 | 80.322 | 0 | 0 | 0 |
| BRACE2008968 | 4.241 | 0 | 0 | 3.369 | 0 | 0 | 0 |
| BRACE2009291 | 0 | 0 | 0 | 7.665 | 0 | 4.583 | 0 |
| BRACE2009517 | 0 | 0 | 0 | 0 | 0 | 9.397 | 0 |
| BRACE2009721 | 0 | 0 | 0 | 0 | 0 | 82.997 | 0 |
| BRACE2010642 | 0 | 0 | 0 | 89.087 | 0 | 0 | 0 |
| BRACE2013911 | 0 | 0 | 0 | 43.556 | 0 | 0 | 0 |
| BRACE2015412 | 0 | 0 | 0 | 70.543 | 0 | 0 | 0 |
| BRACE2017934 | 0 | 0 | 0 | 53.554 | 0 | 0 | 0 |
| BRACE2022638 | 5.437 | 0 | 0 | 0 | 0 | 2.582 | 0 |
| BRACE2028171 | 0 | 0 | 0 | 89.087 | 0 | 0 | 0 |
| BRACE2039624 | 0 | 0 | 0 | 5.606 | 0 | 6.704 | 0 |
| BRACE3010438 | 0 | 0 | 0 | 0 | 0 | 6.204 | 0 |
| BRALZ2009482 | 0 | 0 | 0 | 0 | 0 | 2.575 | 0 |
| BRALZ2014054 | 0 | 0 | 0 | 61.592 | 0 | 0 | 0 |
| BRAMY2000893 | 0 | 0 | 0 | 0 | 0 | 5.272 | 0 |
| BRAMY2000987 | 0 | 0 | 0 | 0 | 0 | 58.805 | 0 |
| BRAMY2004363 | 0 | 0 | 0 | 0 | 0 | 7.083 | 0 |
| BRAMY2004521 | 0 | 0 | 0 | 0 | 0 | 8.212 | 0 |
| BRAMY2012536 | 0 | 50.363 | 0 | 0 | 0 | 0 | 0 |
| BRAMY2016706 | 0 | 0 | 0 | 0 | 0 | 34.345 | 0 |
| BRAMY2019554 | 0 | 0 | 0 | 4.624 | 0 | 0 | 0 |
| BRAMY2022786 | 0 | 0 | 0 | 7.230 | 0 | 8.647 | 0 |
| BRAMY2028740 | 0 | 0 | 0 | 0 | 0 | 6.451 | 0 |
| BRAMY2030545 | 0 | 0 | 0 | 0 | 0 | 77.470 | 0 |
| BRAMY2032087 | 0 | 0 | 0 | 0 | 0 | 4.196 | 0 |
| BRAMY2040095 | 0 | 0 | 0 | 1.981 | 0 | 1.184 | 0 |
| BRAMY3002991 | 0 | 0 | 0 | 22.719 | 0 | 0 | 0 |
| BRAMY3010008 | 0 | 0 | 0 | 0 | 0 | 2.016 | 0 |
| BRAMY4000089 | 0 | 0 | 0 | 29.487 | 0 | 0 | 0 |
| BRAWH1000040 | 0 | 0 | 0 | 0 | 0 | 4.768 | 0 |
| BRAWH1000083 | 0 | 0 | 0 | 0 | 0 | 15.698 | 0 |
| BRAWH2000460 | 0 | 0 | 0 | 7.073 | 0 | 2.115 | 0 |
| BRAWH2000588 | 0 | 0 | 0 | 7.269 | 0 | 8.693 | 0 |
| BRAWH2000697 | 0 | 0 | 0 | 0 | 0 | 3.505 | 0 |
| BRAWH2000839 | 0 | 0 | 0 | 0 | 0 | 1.423 | 0 |
| BRAWH2000924 | 0 | 0 | 0 | 1.663 | 0 | 0.994 | 0 |
| BRAWH2001092 | 0 | 0 | 0 | 5.092 | 0 | 0.609 | 0 |
| BRAWH2004078 | 0 | 0 | 0 | 61.066 | 0 | 0 | 0 |
| BRAWH2005661 | 0 | 0 | 0 | 9.263 | 0 | 0 | 0 |
| BRAWH2009227 | 0 | 0 | 0 | 0 | 0 | 54.758 | 0 |
| BRAWH2010536 | 0 | 0 | 0 | 85.282 | 0 | 0 | 0 |
| BRAWH2017379 | 0 | 0 | 0 | 0 | 0 | 52.645 | 0 |
| BRAWH2017685 | 0 | 0 | 0 | 0 | 0 | 3.517 | 0 |
| BRAWH2018526 | 4.489 | 0 | 0 | 0 | 0 | 3.199 | 0 |
| BRAWH3000088 | 0 | 0 | 0 | 0 | 0 | 4.008 | 0 |
| BRAWH3002574 | 0 | 0 | 0 | 0 | 0 | 22.906 | 0 |
| BRAWH3002853 | 0 | 0 | 0 | 0 | 0 | 2.563 | 0 |
| BRAWH3008931 | 0 | 0 | 0 | 20.069 | 0 | 0 | 0 |
| BRCAN2003944 | 0 | 0 | 0 | 0 | 25.048 | 2.927 | 0 |
| BRCAN2011946 | 0 | 0 | 0 | 36.641 | 0 | 0 | 0 |
| BRCAN2016174 | 0 | 0 | 0 | 62.999 | 0 | 0 | 0 |
| BRCAN2026197 | 0 | 0 | 0 | 0 | 0 | 5.409 | 0 |
| BRCAN2028036 | 0 | 0 | 0 | 13.990 | 0 | 8.365 | 0 |
| BRHIP1000104 | 0 | 0 | 0 | 0 | 0 | 2.265 | 0 |
| BRHIP2000534 | 0 | 0 | 0 | 0 | 0 | 1.186 | 0 |
| BRHIP2000553 | 0 | 0 | 0 | 0 | 0 | 8.903 | 0 |
| BRHIP2007741 | 0 | 0 | 0 | 0 | 0 | 6.014 | 0 |
| BRHIP2023272 | 0 | 0 | 0 | 0 | 0 | 11.694 | 0 |
| BRHIP2023888 | 0 | 0 | 0 | 2.132 | 0 | 3.824 | 0 |
| BRHIP2027077 | 0 | 0 | 0 | 0 | 0 | 6.998 | 0 |
| BRHIP3000111 | 0 | 0 | 0 | 5.932 | 0 | 0 | 0 |
| BRHIP3020046 | 0 | 0 | 0 | 0 | 0 | 3.509 | 0 |
| BRSSN2001869 | 0 | 0 | 0 | 0 | 0 | 10.458 | 0 |
| BRSSN2011262 | 0 | 0 | 0 | 0 | 0 | 0 | 61.476 |
| BRSSN2014299 | 0 | 0 | 0 | 64.234 | 0 | 0 | 0 |
| BRSTN2001067 | 0 | 0 | 0 | 35.873 | 0 | 0 | 0 |
| BRSTN2011961 | 0 | 0 | 0 | 0 | 0 | 0 | 6.317 |
| BRSTN2012069 | 3.312 | 0.825 | 0.919 | 1.699 | 0 | 0.459 | 0 |
| BRSTN2012451 | 0 | 0 | 0 | 0 | 73.463 | 0 | 0 |
| BRSTN2013171 | 0 | 0 | 0 | 40.158 | 0 | 0 | 0 |
| BRSTN2013931 | 0 | 84.303 | 0 | 0 | 0 | 0 | 0 |
| BRSTN2016470 | 0 | 19.098 | 0 | 0 | 0 | 0 | 0 |
| BRTHA2005864 | 0 | 0 | 0 | 0 | 0 | 5.622 | 0 |
| BRTHA3003023 | 0 | 0 | 0 | 0 | 0 | 7.002 | 0 |
| BRTHA3003490 | 0 | 0 | 0 | 0 | 0 | 11.249 | 0 |
| BRTHA3003736 | 0 | 0 | 0 | 0 | 0 | 7.453 | 0 |
| BRTHA3007472 | 0 | 0 | 0 | 0 | 0 | 13.519 | 0 |
| CD34C3000252 | 0 | 0 | 0 | 0 | 0 | 2.844 | 0 |
| CTONG1000087 | 0 | 0 | 0 | 6.059 | 0 | 0 | 0 |
| CTONG1000094 | 2.841 | 0 | 0 | 0 | 0 | 1.349 | 0 |
| CTONG2000469 | 0 | 0 | 0 | 16.920 | 0 | 0 | 0 |
| CTONG2000561 | 0 | 0 | 0 | 0 | 0 | 12.572 | 0 |
| CTONG2002903 | 0 | 0 | 0 | 0 | 0 | 10.672 | 0 |
| CTONG2003094 | 0 | 0 | 0 | 0 | 0 | 12.696 | 0 |
| CTONG2003298 | 0 | 0 | 0 | 0 | 0 | 7.543 | 0 |
| CTONG2003764 | 0 | 0 | 0 | 75.675 | 0 | 0 | 0 |
| CTONG2004115 | 0 | 0 | 0 | 69.255 | 0 | 0 | 0 |
| CTONG2005278 | 0 | 0 | 0 | 0 | 0 | 19.970 | 0 |
| CTONG2005553 | 0 | 0 | 0 | 0 | 0 | 2.481 | 0 |
| CTONG2006932 | 0 | 0 | 0 | 0 | 0 | 3.813 | 0 |
| CTONG2008269 | 0 | 0 | 0 | 39.422 | 0 | 0 | 0 |
| CTONG2008506 | 0 | 0 | 0 | 0 | 0 | 3.260 | 0 |
| CTONG2010348 | 0 | 0 | 0 | 10.260 | 0 | 0 | 0 |
| CTONG2010649 | 0 | 0 | 0 | 0 | 0 | 5.418 | 0 |
| CTONG2011801 | 0 | 0 | 0 | 17.157 | 0 | 0 | 0 |
| CTONG2012452 | 2.597 | 0 | 0 | 2.063 | 0 | 2.467 | 0 |
| CTONG2012453 | 0 | 0 | 0 | 43.638 | 0 | 0 | 0 |
| CTONG2014705 | 0 | 0 | 0 | 0 | 0 | 65.037 | 0 |
| CTONG2015091 | 0 | 0 | 0 | 4.706 | 0 | 0 | 0 |
| CTONG2016185 | 0 | 0 | 0 | 0 | 0 | 9.007 | 0 |
| CTONG2016575 | 3.627 | 0 | 0 | 0 | 0 | 3.445 | 0 |
| CTONG2018383 | 0 | 0 | 0 | 0 | 0 | 15.662 | 0 |
| CTONG2018739 | 0 | 0 | 0 | 0 | 0 | 13.427 | 0 |
| CTONG2018898 | 0 | 0 | 0 | 0 | 0 | 1.669 | 0 |
| CTONG2020484 | 0 | 0 | 0 | 0 | 0 | 2.037 | 0 |
| CTONG2020560 | 0 | 0 | 0 | 0 | 0 | 10.395 | 0 |
| CTONG3009239 | 0 | 0 | 0 | 0 | 0 | 5.002 | 0 |
| D30ST2001670 | 0 | 0 | 0 | 21.620 | 0 | 0 | 0 |
| DFNES2000153 | 0 | 0 | 0 | 18.552 | 0 | 0 | 0 |
| DFNES2000292 | 0 | 0 | 0 | 0 | 0 | 13.003 | 0 |
| DFNES2000443 | 0 | 0 | 0 | 0 | 0 | 2.352 | 0 |
| DFNES2000526 | 0 | 0 | 0 | 0 | 0 | 28.724 | 0 |
| DFNES2001091 | 0 | 0 | 0 | 0 | 0 | 29.734 | 0 |
| DFNES2004371 | 0 | 0 | 0 | 0 | 0 | 5.466 | 0 |
| DFNES2007641 | 0 | 0 | 0 | 0 | 0 | 37.023 | 0 |
| FCBBF1000023 | 0 | 0 | 0 | 0 | 0 | 24.372 | 0 |
| FCBBF1000025 | 0 | 0 | 0 | 0 | 0 | 11.087 | 0 |
| FCBBF1000063 | 0 | 0 | 0 | 0 | 0 | 20.721 | 0 |
| FCBBF1000367 | 0 | 0 | 0 | 8.803 | 0 | 5.263 | 0 |
| FCBBF1000466 | 0 | 0 | 0 | 0 | 0 | 2.206 | 0 |
| FCBBF1000760 | 0 | 0 | 0 | 0 | 58.246 | 0 | 0 |
| FCBBF2000087 | 0 | 0 | 0 | 0 | 0 | 9.995 | 0 |
| FCBBF2000502 | 0 | 0 | 0 | 38.225 | 0 | 0 | 0 |
| FCBBF2000591 | 0 | 0 | 0 | 0 | 0 | 0.993 | 0 |
| FCBBF2000885 | 0 | 0 | 0 | 0 | 0 | 5.318 | 0 |
| FCBBF2001105 | 0 | 0 | 0 | 17.589 | 0 | 0 | 0 |
| FCBBF3001632 | 0 | 0 | 0 | 0 | 0 | 5.348 | 0 |
| FCBBF3003800 | 2.873 | 0 | 0 | 0 | 0 | 1.365 | 0 |
| FCBBF3004473 | 0 | 0 | 0 | 0 | 0 | 3.980 | 0 |
| FCBBF3005320 | 0 | 0 | 0 | 0 | 0 | 9.451 | 0 |
| FCBBF3005497 | 0 | 0 | 0 | 0 | 0 | 2.724 | 0 |
| FCBBF3016928 | 0 | 0 | 0 | 0 | 0 | 3.036 | 0 |
| FCBBF3019839 | 0 | 0 | 0 | 0 | 0 | 12.975 | 0 |
| FCBBF3023667 | 3.875 | 11.587 | 0 | 1.539 | 11.811 | 0 | 0 |
| FCBBF4000446 | 0 | 0 | 0 | 0 | 0 | 5.205 | 0 |
| FEBRA1000057 | 0 | 0 | 0 | 0 | 0 | 6.015 | 0 |
| FEBRA2000129 | 0 | 0 | 0 | 15.063 | 0 | 0 | 0 |
| FEBRA2000438 | 0 | 0 | 0 | 51.194 | 0 | 0 | 0 |
| FEBRA2000733 | 1.229 | 0 | 0 | 1.953 | 0 | 0.584 | 0 |
| FEBRA2000762 | 0 | 0 | 0 | 36.045 | 0 | 0 | 0 |
| FEBRA2000772 | 0 | 0 | 0 | 49.565 | 0 | 0 | 0 |
| FEBRA2000881 | 0 | 0 | 0 | 18.341 | 0 | 0 | 0 |
| FEBRA2001245 | 0 | 0 | 0 | 0 | 0 | 8.739 | 0 |
| FEBRA2001294 | 0 | 0 | 0 | 0 | 0 | 8.373 | 0 |
| FEBRA2001828 | 0 | 0 | 0 | 31.524 | 0 | 0 | 0 |
| FEBRA2002352 | 0 | 0 | 0 | 0 | 0 | 12.027 | 0 |
| FEBRA2002707 | 0 | 0 | 0 | 20.879 | 0 | 0 | 0 |
| FEBRA2002933 | 0 | 0 | 0 | 2.144 | 0 | 1.282 | 0 |
| FEBRA2003100 | 0 | 0 | 0 | 0 | 0 | 2.282 | 0 |
| FEBRA2003726 | 0 | 0 | 0 | 0 | 0 | 3.450 | 0 |
| FEBRA2003750 | 0 | 0 | 0 | 0 | 0 | 6.325 | 0 |
| FEBRA2003926 | 0 | 0 | 0 | 4.045 | 0 | 2.419 | 0 |
| FEBRA2004224 | 0 | 0 | 0 | 13.416 | 0 | 4.011 | 0 |
| FEBRA2004325 | 0 | 0 | 0 | 0 | 0 | 12.203 | 0 |
| FEBRA2004628 | 0 | 0 | 0 | 16.528 | 0 | 0 | 0 |
| FEBRA2005416 | 0 | 0 | 0 | 0 | 0 | 3.721 | 0 |
| FEBRA2005824 | 0 | 0 | 0 | 2.161 | 0 | 0 | 0 |
| FEBRA2006092 | 0 | 0 | 0 | 0 | 55.306 | 0 | 0 |
| FEBRA2006372 | 0 | 0 | 0 | 0 | 0 | 4.653 | 0 |
| FEBRA2006401 | 0 | 0 | 0 | 0 | 0 | 0.870 | 0 |
| FEBRA2006485 | 0 | 0 | 0 | 0 | 0 | 10.539 | 0 |
| FEBRA2006873 | 0 | 0 | 0 | 0 | 0 | 31.115 | 0 |
| FEBRA2007818 | 40.789 | 0 | 0 | 32.401 | 0 | 0 | 0 |
| FEBRA2008081 | 0 | 0 | 0 | 0 | 0 | 2.453 | 0 |
| FEBRA2008087 | 0 | 0 | 0 | 2.077 | 0 | 0.621 | 0 |
| FEBRA2008302 | 0 | 0 | 0 | 0 | 0 | 3.687 | 0 |
| FEBRA2008341 | 0 | 0 | 0 | 38.062 | 0 | 0 | 0 |
| FEBRA2008583 | 0.964 | 0 | 0 | 0 | 0 | 1.832 | 0 |
| FEBRA2014417 | 0 | 0 | 0 | 3.129 | 0 | 1.871 | 0 |
| FEBRA2018476 | 0 | 0 | 0 | 6.331 | 0 | 0 | 0 |
| FEBRA2022504 | 0 | 0 | 0 | 0 | 0 | 5.690 | 0 |
| FEBRA2025838 | 0 | 0 | 0 | 0 | 0 | 2.846 | 0 |
| FEBRA2028516 | 0 | 0 | 0 | 6.628 | 0 | 0 | 0 |
| FELIV1000153 | 1.203 | 0 | 0 | 0 | 0 | 0.571 | 0 |
| HCASM2005388 | 0 | 0 | 0 | 0 | 0 | 12.254 | 0 |
| HCASM2009588 | 0 | 0 | 0 | 0 | 93.054 | 0 | 0 |
| HCHON1000030 | 0 | 0 | 0 | 2.102 | 0 | 0.628 | 0 |
| HCHON1000131 | 0 | 0 | 0 | 23.773 | 0 | 0 | 0 |
| HCHON1000191 | 0 | 0 | 0 | 0 | 0 | 10.041 | 0 |
| HCHON2000056 | 0 | 0 | 0 | 3.287 | 0 | 0.983 | 0 |
| HCHON2000062 | 0 | 0 | 0 | 24.285 | 0 | 0 | 0 |
| HCHON2000295 | 0 | 0 | 0 | 0 | 0 | 3.035 | 0 |
| HCHON2000323 | 0 | 0 | 0 | 0 | 0 | 6.418 | 0 |
| HCHON2000373 | 0 | 0 | 0 | 0 | 0 | 35.114 | 0 |
| HCHON2000676 | 0 | 0 | 0 | 0 | 0 | 10.330 | 0 |
| HCHON2000698 | 0 | 0 | 0 | 0 | 0 | 9.931 | 0 |
| HCHON2000826 | 0 | 0 | 0 | 0 | 0 | 4.959 | 0 |
| HCHON2001039 | 0 | 0 | 0 | 0 | 0 | 3.866 | 0 |
| HCHON2001050 | 0 | 0 | 0 | 8.398 | 0 | 0 | 0 |
| HCHON2001110 | 0 | 0 | 0 | 47.801 | 0 | 0 | 0 |
| HCHON2001200 | 0 | 0 | 0 | 0 | 0 | 2.997 | 0 |
| HCHON2001217 | 0 | 0 | 0 | 0 | 0 | 3.606 | 0 |
| HCHON2001407 | 0 | 0 | 0 | 0 | 0 | 2.736 | 0 |
| HCHON2001434 | 0 | 0 | 0 | 0 | 0 | 5.293 | 0 |
| HEART1000074 | 11.074 | 0 | 0 | 4.399 | 0 | 0 | 0 |
| HEART1000149 | 0 | 0 | 0 | 5.441 | 0 | 0 | 0 |
| HHDPC1000083 | 0 | 0 | 0 | 0 | 0 | 6.782 | 0 |
| HHDPC1000163 | 0 | 0 | 0 | 0 | 0 | 8.714 | 0 |
| HHDPC2000102 | 0 | 0 | 0 | 0 | 0 | 16.450 | 0 |
| HHDPC2000115 | 0 | 0 | 0 | 15.158 | 0 | 0 | 0 |
| HHDPC2000455 | 0 | 0 | 0 | 0 | 0 | 1.904 | 0 |
| HHDPC2000456 | 0 | 0 | 0 | 0 | 0 | 5.379 | 0 |
| HHDPC2000656 | 0 | 0 | 0 | 0 | 0 | 29.616 | 0 |
| HHDPC2003439 | 1.855 | 0 | 0 | 0 | 0 | 3.525 | 0 |
| HHDPC2003983 | 2.031 | 0 | 0 | 0 | 0 | 2.894 | 0 |
| HHDPC2006862 | 0 | 0 | 0 | 0 | 0 | 25.931 | 0 |
| HLUNG1000055 | 0 | 0 | 0 | 0 | 0 | 1.875 | 0 |
| HLUNG1000076 | 0 | 0 | 0 | 4.875 | 0 | 0 | 0 |
| HLUNG1000105 | 0 | 0 | 0 | 61.237 | 0 | 0 | 0 |
| HLUNG2001439 | 0 | 0 | 0 | 0 | 0 | 3.055 | 0 |
| HLUNG2001507 | 0 | 0 | 0 | 0 | 0 | 52.047 | 0 |
| HLUNG2001518 | 7.976 | 0 | 0 | 6.336 | 0 | 0 | 0 |
| HLUNG2002648 | 0 | 0 | 0 | 20.943 | 0 | 0 | 0 |
| HLUNG2002916 | 0 | 0 | 0 | 49.989 | 0 | 0 | 0 |
| HSYRA1000062 | 0 | 0 | 0 | 0 | 0 | 1.484 | 0 |
| HSYRA1000119 | 0 | 0 | 0 | 5.328 | 0 | 1.593 | 0 |
| HSYRA1000148 | 0 | 0 | 0 | 38.210 | 0 | 0 | 0 |
| HSYRA2000135 | 0 | 0 | 0 | 0 | 0 | 13.703 | 0 |
| HSYRA2000159 | 0.879 | 0 | 0 | 0 | 0 | 2.089 | 0 |
| HSYRA2000232 | 0 | 0 | 0 | 8.569 | 0 | 0 | 0 |
| HSYRA2000248 | 0 | 0 | 0 | 3.938 | 0 | 2.355 | 0 |
| HSYRA2000828 | 0 | 0 | 0 | 0 | 0 | 4.936 | 0 |
| HSYRA2000925 | 0 | 0 | 0 | 1.284 | 0 | 0 | 0 |
| HSYRA2001003 | 0 | 0 | 0 | 0 | 0 | 29.743 | 0 |
| HSYRA2001138 | 0 | 0 | 0 | 0 | 22.675 | 4.416 | 0 |
| HSYRA2001153 | 0 | 0 | 0 | 0 | 0 | 31.673 | 0 |
| HSYRA2001232 | 0 | 0 | 0 | 0 | 0 | 13.403 | 0 |
| HSYRA2001476 | 0 | 0 | 0 | 0 | 0 | 1.673 | 0 |
| HSYRA2001595 | 0 | 0 | 0 | 0 | 0 | 1.974 | 0 |
| HSYRA2001631 | 0 | 0 | 0 | 0 | 0 | 20.202 | 0 |
| HSYRA2001638 | 0 | 0 | 0 | 0 | 0 | 7.836 | 0 |
| IMR321000158 | 5.552 | 0 | 0 | 0 | 0 | 2.637 | 0 |
| IMR321000219 | 2.047 | 0 | 0 | 0 | 0 | 2.917 | 0 |
| IMR321000266 | 0 | 0 | 0 | 0 | 0 | 19.609 | 0 |
| IMR322000730 | 2.000 | 0 | 0 | 1.588 | 0 | 0 | 0 |
| IMR322000791 | 0 | 0 | 0 | 30.062 | 0 | 0 | 0 |
| IMR322000973 | 0 | 0 | 0 | 0 | 0 | 8.381 | 0 |
| IMR322001435 | 0 | 0 | 0 | 0 | 0 | 3.255 | 0 |
| IMR322001600 | 0 | 0 | 0 | 0 | 0 | 1.116 | 0 |
| IMR322012529 | 0 | 0 | 0 | 0 | 0 | 16.315 | 0 |
| KIDNE2000085 | 0 | 0 | 0 | 5.958 | 0 | 0 | 0 |
| KIDNE2000574 | 0 | 0 | 0 | 0 | 0 | 2.593 | 0 |
| KIDNE2000665 | 0 | 0 | 0 | 11.217 | 0 | 0 | 0 |
| KIDNE2000909 | 0 | 0 | 0 | 0 | 0 | 7.113 | 0 |
| KIDNE2000947 | 0 | 0 | 0 | 0 | 0 | 8.756 | 0 |
| KIDNE2001467 | 0 | 0 | 0 | 0 | 0 | 3.612 | 0 |
| KIDNE2002839 | 0 | 0 | 0 | 0 | 0 | 25.434 | 0 |
| KIDNE2003941 | 0 | 0 | 0 | 0 | 0 | 1.748 | 0 |
| KIDNE2004054 | 0 | 0 | 0 | 0 | 0 | 45.226 | 0 |
| KIDNE2004534 | 0 | 0 | 0 | 0 | 0 | 12.932 | 0 |
| KIDNE2005676 | 0 | 0 | 0 | 0 | 0 | 4.874 | 0 |
| KIDNE2006030 | 0 | 0 | 0 | 0 | 0 | 14.787 | 0 |
| KIDNE2007352 | 0 | 0 | 0 | 2.409 | 0 | 1.440 | 0 |
| KIDNE2007811 | 0 | 0 | 0 | 0 | 0 | 29.682 | 0 |
| KIDNE2008048 | 0 | 0 | 0 | 0 | 0 | 1.859 | 0 |
| KIDNE2012440 | 0 | 0 | 0 | 0 | 0 | 6.133 | 0 |
| KIDNE2013845 | 0 | 0 | 0 | 0 | 0 | 3.872 | 0 |
| KIDNE2015710 | 0 | 0 | 0 | 0 | 0 | 0 | 40.832 |
| KIDNE2018352 | 0 | 0 | 0 | 0 | 0 | 12.355 | 0 |
| LIVER1000017 | 0 | 0 | 0 | 0 | 0 | 0 | 25.940 |
| LIVER1000067 | 0 | 0 | 0 | 0 | 0 | 0 | 30.284 |
| LIVER1000079 | 0 | 0 | 0 | 10.153 | 0 | 0 | 0 |
| LIVER1000104 | 0 | 0 | 0 | 0 | 0 | 0 | 26.973 |
| LIVER1000111 | 0 | 0 | 0 | 0 | 0 | 21.880 | 0 |
| LIVER1000132 | 0 | 0 | 0 | 0 | 0 | 5.201 | 0 |
| LIVER1000542 | 0 | 0 | 0 | 0 | 0 | 0 | 64.596 |
| LIVER2000037 | 0 | 0 | 0 | 0 | 0 | 0 | 78.666 |
| LIVER2000158 | 0 | 0 | 0 | 40.064 | 0 | 0 | 0 |
| LIVER2001608 | 0 | 0 | 0 | 0 | 0 | 4.550 | 0 |
| LIVER2002842 | 0 | 0 | 0 | 11.062 | 0 | 0 | 0 |
| LIVER2003800 | 0 | 0 | 0 | 0 | 0 | 0 | 90.963 |
| LIVER2006006 | 0 | 0 | 0 | 0 | 0 | 4.928 | 0 |
| MAMGL1000032 | 0 | 0 | 0 | 0 | 0 | 0.558 | 0 |
| MAMGL1000035 | 0 | 0 | 0 | 0 | 0 | 0.698 | 0 |
| MAMGL1000100 | 0 | 0 | 0 | 1.711 | 0 | 0 | 0 |
| MAMGL1000173 | 0 | 0 | 0 | 3.072 | 0 | 0 | 0 |
| MESAN1000050 | 0 | 0 | 0 | 0 | 0 | 11.124 | 0 |
| MESAN1000080 | 0 | 0 | 0 | 0 | 0 | 9.893 | 0 |
| MESAN2000149 | 0 | 0 | 0 | 0 | 0 | 8.511 | 0 |
| MESAN2000267 | 5.974 | 0 | 0 | 0 | 0 | 4.256 | 0 |
| MESAN2000572 | 7.457 | 0 | 0 | 0 | 0 | 10.626 | 0 |
| MESAN2000608 | 0 | 0 | 0 | 0 | 0 | 2.724 | 0 |
| MESAN2002086 | 0 | 0 | 0 | 2.412 | 0 | 5.769 | 0 |
| MESAN2002940 | 0 | 0 | 0 | 0 | 0 | 11.019 | 0 |
| MESAN2003444 | 0 | 0 | 0 | 0 | 0 | 12.906 | 0 |
| MESAN2003622 | 0 | 0 | 0 | 6.964 | 0 | 0 | 0 |
| MESAN2005766 | 0 | 0 | 0 | 0 | 0 | 3.417 | 0 |
| MESAN2006743 | 0 | 0 | 0 | 0 | 0 | 13.355 | 0 |
| MESAN2006953 | 0 | 0 | 0 | 0 | 0 | 47.513 | 0 |
| MESAN2008334 | 0 | 0 | 0 | 47.634 | 0 | 0 | 0 |
| MESAN2009580 | 0 | 0 | 0 | 0 | 0 | 3.848 | 0 |
| MESAN2013284 | 0 | 0 | 0 | 0 | 0 | 4.255 | 0 |
| MESAN2013845 | 0 | 0 | 0 | 0 | 0 | 19.332 | 0 |
| MESAN2018576 | 0 | 0 | 0 | 60.977 | 0 | 0 | 0 |
| NB9N41000340 | 0 | 0 | 0 | 8.403 | 0 | 0 | 0 |
| NHNPC2000062 | 0 | 0 | 0 | 12.143 | 0 | 0 | 0 |
| NHNPC2000187 | 3.301 | 0 | 0 | 0 | 0 | 1.568 | 0 |
| NHNPC2000206 | 0 | 0 | 0 | 0 | 0 | 4.758 | 0 |
| NHNPC2001931 | 0 | 0 | 0 | 0 | 0 | 3.307 | 0 |
| NOVAR1000091 | 0 | 0 | 0 | 0 | 0 | 6.709 | 0 |
| NOVAR2000038 | 0 | 0 | 0 | 3.599 | 0 | 0 | 0 |
| NT2NE1000163 | 0 | 0 | 39.372 | 0 | 0 | 2.807 | 0 |
| NT2NE2000056 | 0 | 0 | 0 | 0 | 0 | 2.722 | 0 |
| NT2NE2000174 | 2.783 | 0 | 0 | 0 | 33.939 | 0 | 0 |
| NT2NE2000259 | 0 | 0 | 0 | 16.299 | 0 | 9.746 | 0 |
| NT2NE2000260 | 0 | 0 | 0 | 0 | 0 | 0.419 | 0 |
| NT2NE2000383 | 0 | 0 | 0 | 0 | 0 | 5.265 | 0 |
| NT2NE2000384 | 0 | 0 | 0 | 0 | 0 | 15.948 | 0 |
| NT2NE2000575 | 5.261 | 0 | 0 | 2.786 | 0 | 0 | 0 |
| NT2NE2000636 | 0 | 0 | 0 | 0 | 0 | 25.020 | 0 |
| NT2NE2000707 | 0 | 0 | 0 | 0 | 0 | 2.319 | 0 |
| NT2NE2000809 | 0 | 0 | 0 | 19.640 | 0 | 0 | 0 |
| NT2NE2000963 | 0 | 0 | 0 | 0 | 0 | 0.795 | 0 |
| NT2NE2001000 | 0 | 0 | 0 | 4.987 | 0 | 0 | 0 |
| NT2NE2001048 | 0 | 0 | 0 | 8.673 | 0 | 0 | 0 |
| NT2NE2005688 | 0 | 0 | 0 | 0 | 0 | 2.587 | 0 |
| NT2NE2006458 | 0 | 0 | 0 | 0 | 0 | 4.897 | 0 |
| NT2NE2010842 | 0 | 0 | 0 | 0 | 0 | 3.267 | 0 |
| NT2NE2014104 | 0 | 0 | 0 | 0 | 0 | 5.815 | 0 |
| NT2RI1000035 | 0 | 0 | 0 | 0 | 0 | 65.595 | 0 |
| NT2RI1000048 | 0 | 0 | 0 | 0 | 0 | 2.942 | 0 |
| NT2RI2000064 | 0 | 0 | 0 | 6.238 | 0 | 0 | 0 |
| NT2RI2000282 | 4.618 | 0 | 0 | 0 | 0 | 3.290 | 0 |
| NT2RI2000284 | 0 | 39.777 | 0 | 5.283 | 0 | 0 | 0 |
| NT2RI2000578 | 2.439 | 0 | 0 | 1.938 | 0 | 0 | 0 |
| NT2RI2000671 | 0 | 0 | 0 | 6.095 | 0 | 0 | 0 |
| NT2RI2000727 | 0 | 0 | 0 | 0 | 0 | 0.896 | 0 |
| NT2RI2000974 | 0 | 0 | 0 | 0 | 0 | 7.473 | 0 |
| NT2RI2001054 | 0 | 0 | 0 | 0 | 52.921 | 0 | 0 |
| NT2RI2001342 | 0 | 0 | 0 | 0 | 0 | 5.469 | 0 |
| NT2RI2001385 | 0 | 0 | 0 | 22.829 | 0 | 0 | 0 |
| NT2RI2001540 | 2.076 | 0 | 5.533 | 2.639 | 0 | 0.394 | 0 |
| NT2RI2002091 | 0.978 | 1.170 | 0 | 1.554 | 4.772 | 2.091 | 0 |
| NT2RI2002117 | 0 | 0 | 0 | 27.298 | 0 | 0 | 0 |
| NT2RI2002152 | 0 | 0 | 0 | 0 | 0 | 4.357 | 0 |
| NT2RI2002316 | 0 | 0 | 0 | 0 | 0 | 2.778 | 0 |
| NT2RI2002654 | 0 | 0 | 0 | 0 | 0 | 2.035 | 0 |
| NT2RI2002822 | 0 | 0 | 0 | 0 | 0 | 34.766 | 0 |
| NT2RI2002958 | 0 | 0 | 0 | 0 | 0 | 3.336 | 0 |
| NT2RI2003051 | 0 | 0 | 0 | 5.932 | 0 | 3.547 | 0 |
| NT2RI2003304 | 0 | 0 | 0 | 0 | 0 | 1.818 | 0 |
| NT2RI2003338 | 0 | 0 | 0 | 0 | 0 | 3.508 | 0 |
| NT2RI2003407 | 0 | 0 | 0 | 0 | 0 | 4.193 | 0 |
| NT2RI2003420 | 0 | 0 | 0 | 0 | 0 | 5.335 | 0 |
| NT2RI2003556 | 0 | 33.196 | 0 | 2.204 | 0 | 1.318 | 0 |
| NT2RI2003678 | 0 | 0 | 0 | 0 | 0 | 6.597 | 0 |
| NT2RI2003738 | 2.552 | 15.264 | 0 | 1.014 | 0 | 0.606 | 0 |
| NT2RI2003751 | 0 | 0 | 0 | 0 | 0 | 4.800 | 0 |
| NT2RI2003993 | 0 | 0 | 0 | 21.997 | 0 | 13.153 | 0 |
| NT2RI2004230 | 0 | 0 | 0 | 0 | 0 | 3.975 | 0 |
| NT2RI2004535 | 0 | 0 | 0 | 0 | 0 | 13.264 | 0 |
| NT2RI2004884 | 0 | 0 | 0 | 0 | 0 | 2.677 | 0 |
| NT2RI2005335 | 0 | 0 | 0 | 0 | 0 | 2.682 | 0 |
| NT2RI2005353 | 0 | 0 | 0 | 0 | 0 | 3.991 | 0 |
| NT2RI2005405 | 0 | 0 | 0 | 0 | 0 | 3.890 | 0 |
| NT2RI2005710 | 0 | 0 | 0 | 0 | 0 | 18.085 | 0 |
| NT2RI2005723 | 0 | 0 | 0 | 0 | 0 | 1.739 | 0 |
| NT2RI2005814 | 0 | 0 | 0 | 0 | 0 | 4.512 | 0 |
| NT2RI2005818 | 0 | 0 | 0 | 0 | 0 | 8.824 | 0 |
| NT2RI2006072 | 0 | 0 | 0 | 0 | 0 | 2.651 | 0 |
| NT2RI2007054 | 0 | 0 | 0 | 0 | 0 | 0 | 28.320 |
| NT2RI2007084 | 0 | 0 | 0 | 6.304 | 0 | 0 | 0 |
| NT2RI2007384 | 0 | 0 | 0 | 0 | 0 | 4.501 | 0 |
| NT2RI2007445 | 0 | 0 | 0 | 0 | 0 | 6.212 | 0 |
| NT2RI2007572 | 0 | 0 | 0 | 0 | 0 | 2.589 | 0 |
| NT2RI2007593 | 0 | 0 | 0 | 0 | 0 | 22.830 | 0 |
| NT2RI2007629 | 0 | 0 | 0 | 11.680 | 0 | 6.984 | 0 |
| NT2RI2007879 | 0 | 0 | 0 | 0 | 0 | 5.499 | 0 |
| NT2RI2008045 | 5.664 | 0 | 0 | 0 | 0 | 5.380 | 0 |
| NT2RI2008141 | 0 | 0 | 0 | 0 | 0 | 7.408 | 0 |
| NT2RI2008233 | 0 | 0 | 0 | 0 | 0 | 65.595 | 0 |
| NT2RI2008396 | 0 | 0 | 0 | 2.554 | 0 | 4.582 | 0 |
| NT2RI2008526 | 0 | 0 | 0 | 0 | 0 | 5.313 | 0 |
| NT2RI2008598 | 0 | 0 | 0 | 0 | 0 | 29.063 | 0 |
| NT2RI2008656 | 0 | 0 | 0 | 0 | 0 | 9.409 | 0 |
| NT2RI2008724 | 0 | 0 | 0 | 0 | 0 | 6.363 | 0 |
| NT2RI2008749 | 0 | 0 | 0 | 3.276 | 0 | 0 | 0 |
| NT2RI2008791 | 0 | 0 | 0 | 20.281 | 0 | 0 | 0 |
| NT2RI2009194 | 0 | 0 | 0 | 0 | 0 | 3.405 | 0 |
| NT2RI2009233 | 0 | 0 | 0 | 0 | 0 | 7.815 | 0 |
| NT2RI2009269 | 0 | 0 | 0 | 0 | 0 | 3.424 | 0 |
| NT2RI2009583 | 0 | 0 | 0 | 0 | 0 | 0 | 4.881 |
| NT2RI2012350 | 0 | 0 | 0 | 2.319 | 0 | 0 | 0 |
| NT2RI3007543 | 0 | 0 | 0 | 0 | 0 | 2.940 | 0 |
| NT2RP6000123 | 5.711 | 0 | 0 | 0 | 0 | 1.356 | 0 |
| NT2RP7000906 | 0 | 0 | 0 | 0 | 0 | 4.520 | 0 |
| NT2RP7002028 | 0 | 0 | 0 | 0 | 0 | 6.962 | 0 |
| NT2RP7003319 | 0 | 0 | 0 | 0 | 0 | 3.161 | 0 |
| NT2RP7003632 | 0 | 0 | 0 | 0 | 0 | 0 | 35.519 |
| NT2RP7003960 | 0 | 0 | 0 | 0 | 0 | 5.740 | 0 |
| NT2RP7004123 | 0 | 0 | 0 | 0 | 0 | 6.649 | 0 |
| NT2RP7004173 | 5.571 | 0 | 0 | 4.425 | 0 | 0 | 0 |
| NT2RP7004204 | 0 | 0 | 0 | 0 | 0 | 5.908 | 0 |
| NT2RP7004373 | 0 | 0 | 0 | 6.820 | 0 | 0 | 0 |
| NT2RP7004428 | 0 | 0 | 0 | 0 | 0 | 11.684 | 0 |
| NT2RP7004728 | 0 | 0 | 0 | 9.107 | 0 | 0 | 0 |
| NT2RP7004751 | 0 | 0 | 0 | 0 | 0 | 1.622 | 0 |
| NT2RP7004790 | 0 | 0 | 0 | 0 | 0 | 27.039 | 0 |
| NT2RP7004946 | 0 | 0 | 0 | 0 | 0 | 5.965 | 0 |
| NT2RP7006075 | 0 | 0 | 0 | 15.181 | 0 | 0 | 0 |
| NT2RP7006141 | 0 | 0 | 0 | 0 | 0 | 0 | 16.109 |
| NT2RP7006263 | 0 | 0 | 0 | 0 | 0 | 13.951 | 0 |
| NT2RP7006619 | 0 | 0 | 0 | 25.579 | 0 | 0 | 0 |
| NT2RP7006717 | 0 | 0 | 0 | 0 | 0 | 4.559 | 0 |
| NT2RP7006986 | 0 | 0 | 0 | 0 | 0 | 13.015 | 0 |
| NT2RP7007154 | 0 | 0 | 0 | 0 | 50.720 | 1.976 | 0 |
| NT2RP7007177 | 0 | 0 | 0 | 0 | 0 | 4.718 | 0 |
| NT2RP7007387 | 0 | 0 | 0 | 0 | 0 | 7.111 | 0 |
| NT2RP7008190 | 0 | 0 | 0 | 0 | 0 | 4.596 | 0 |
| NT2RP7009097 | 0 | 0 | 0 | 0 | 0 | 6.229 | 0 |
| NT2RP7009259 | 0 | 0 | 0 | 3.255 | 0 | 3.893 | 0 |
| NT2RP7009481 | 0 | 0 | 0 | 23.730 | 0 | 0 | 0 |
| NT2RP7014910 | 8.721 | 0 | 0 | 0 | 0 | 4.142 | 0 |
| NT2RP7015512 | 0 | 0 | 0 | 0 | 0 | 22.394 | 0 |
| NT2RP7019835 | 0 | 0 | 0 | 0 | 92.689 | 0 | 0 |
| NT2RP8001604 | 0 | 0 | 0 | 0 | 0 | 24.040 | 0 |
| NTONG1000047 | 0 | 0 | 0 | 34.814 | 0 | 0 | 0 |
| NTONG1000098 | 0 | 0 | 0 | 0 | 0 | 4.415 | 0 |
| NTONG2000499 | 0 | 0 | 0 | 0 | 0 | 31.978 | 0 |
| NTONG2000876 | 10.157 | 0 | 0 | 0 | 0 | 4.824 | 0 |
| NTONG2000977 | 0 | 0 | 0 | 0 | 0 | 3.406 | 0 |
| NTONG2001550 | 0 | 0 | 0 | 0 | 0 | 24.117 | 0 |
| NTONG2001567 | 0 | 0 | 0 | 0 | 0 | 1.062 | 0 |
| NTONG2003839 | 0 | 0 | 0 | 0 | 0 | 18.168 | 0 |
| NTONG2004614 | 0 | 0 | 0 | 34.313 | 0 | 0 | 0 |
| NTONG2008483 | 0 | 0 | 0 | 0 | 0 | 5.536 | 0 |
| OCBBF1000114 | 0 | 0 | 0 | 14.771 | 0 | 0 | 0 |
| OCBBF2000013 | 0 | 0 | 0 | 0 | 0 | 6.742 | 0 |
| OCBBF2000522 | 0 | 0 | 0 | 0 | 0 | 8.761 | 0 |
| OCBBF2000846 | 0 | 0 | 0 | 0 | 0 | 2.535 | 0 |
| OCBBF2001492 | 0 | 0 | 0 | 0 | 0 | 2.377 | 0 |
| OCBBF2004418 | 0 | 0 | 0 | 82.345 | 0 | 0 | 0 |
| OCBBF2005476 | 0 | 0 | 0 | 54.176 | 0 | 0 | 0 |
| OCBBF2007354 | 0 | 0 | 0 | 0 | 0 | 3.481 | 0 |
| OCBBF2007829 | 0 | 0 | 0 | 3.181 | 0 | 0 | 0 |
| OCBBF2007892 | 0 | 0 | 0 | 0 | 0 | 14.759 | 0 |
| OCBBF2009301 | 0 | 0 | 0 | 0 | 0 | 7.182 | 0 |
| OCBBF2009772 | 0 | 0 | 0 | 0 | 0 | 4.080 | 0 |
| OCBBF2011914 | 0 | 0 | 0 | 21.128 | 0 | 12.633 | 0 |
| OCBBF2014064 | 0 | 0 | 0 | 15.828 | 0 | 0 | 0 |
| OCBBF2014292 | 0 | 0 | 0 | 0 | 0 | 7.177 | 0 |
| OCBBF2016729 | 0 | 0 | 0 | 0 | 0 | 31.583 | 0 |
| OCBBF2018707 | 0 | 0 | 0 | 0 | 0 | 7.792 | 0 |
| OCBBF2018956 | 0 | 0 | 0 | 0 | 0 | 5.864 | 0 |
| OCBBF2035226 | 0 | 51.258 | 0 | 0 | 0 | 0 | 0 |
| OCBBF2038317 | 0 | 0 | 0 | 57.112 | 0 | 0 | 0 |
| PEBLM2000147 | 0 | 0 | 0 | 0 | 0 | 8.840 | 0 |
| PEBLM2000308 | 0 | 0 | 0 | 0 | 0 | 13.737 | 0 |
| PEBLM2000395 | 0 | 0 | 0 | 0 | 0 | 3.153 | 0 |
| PEBLM2007774 | 0 | 0 | 0 | 1.745 | 0 | 2.087 | 0 |
| PERIC2007068 | 0 | 0 | 0 | 0 | 0 | 5.346 | 0 |
| PLACE6000145 | 0 | 0 | 0 | 6.005 | 0 | 3.591 | 0 |
| PLACE6000263 | 0 | 0 | 0 | 26.429 | 0 | 5.268 | 0 |
| PLACE6000348 | 0 | 0 | 0 | 0 | 0 | 49.326 | 0 |
| PLACE6000379 | 0 | 0 | 0 | 0 | 0 | 2.448 | 0 |
| PLACE6001064 | 0 | 0 | 0 | 0 | 0 | 1.988 | 0 |
| PLACE6001923 | 0 | 0 | 0 | 0 | 0 | 21.777 | 0 |
| PLACE6002084 | 0 | 0 | 0 | 10.700 | 0 | 5.118 | 0 |
| PLACE6002668 | 0 | 0 | 0 | 62.541 | 0 | 0 | 0 |
| PLACE6004005 | 0 | 0 | 0 | 5.721 | 0 | 0 | 0 |
| PLACE6004578 | 0 | 0 | 0 | 50.277 | 0 | 0 | 0 |
| PLACE6004993 | 0 | 0 | 0 | 0 | 0 | 66.064 | 0 |
| PLACE6006042 | 0 | 0 | 0 | 0 | 0 | 18.861 | 0 |
| PLACE6006549 | 0 | 0 | 0 | 0 | 0 | 2.767 | 0 |
| PLACE6007925 | 0 | 0 | 0 | 58.717 | 0 | 0 | 0 |
| PLACE6008793 | 0 | 0 | 0 | 9.187 | 0 | 0 | 0 |
| PLACE6010077 | 0 | 0 | 0 | 0 | 0 | 9.050 | 0 |
| PLACE6018487 | 0 | 0 | 64.866 | 0 | 0 | 0 | 0 |
| PLACE6020145 | 0 | 0 | 0 | 0 | 0 | 29.076 | 0 |
| PLACE7004103 | 0 | 0 | 0 | 0 | 0 | 4.481 | 0 |
| PROST1000085 | 0 | 0 | 0 | 0 | 0 | 23.969 | 0 |
| PROST1000120 | 0 | 0 | 0 | 0 | 0 | 11.425 | 0 |
| PROST1000526 | 0 | 0 | 0 | 0 | 0 | 17.507 | 0 |
| PROST1000564 | 0 | 0 | 0 | 0 | 0 | 9.315 | 0 |
| PROST2000138 | 0 | 0 | 0 | 0 | 0 | 0.300 | 0 |
| PROST2000176 | 0 | 0 | 0 | 0 | 0 | 0 | 55.365 |
| PROST2000760 | 0 | 0 | 0 | 0 | 0 | 9.719 | 0 |
| PROST2001739 | 0 | 0 | 0 | 76.613 | 0 | 0 | 0 |
| PROST2002927 | 0 | 0 | 0 | 62.033 | 0 | 0 | 0 |
| PROST2003232 | 0 | 59.533 | 0 | 7.907 | 0 | 0 | 0 |
| PROST2003517 | 0 | 0 | 0 | 15.255 | 0 | 9.121 | 0 |
| PROST2004095 | 0 | 0 | 0 | 0 | 0 | 3.243 | 0 |
| PROST2006536 | 0 | 0 | 0 | 0 | 0 | 7.688 | 0 |
| PROST2006737 | 0 | 0 | 0 | 14.044 | 0 | 0 | 0 |
| PROST2007612 | 23.293 | 0 | 0 | 0 | 0 | 11.064 | 0 |
| PROST2008489 | 0 | 0 | 0 | 3.753 | 0 | 4.488 | 0 |
| PROST2008770 | 0 | 0 | 0 | 4.520 | 0 | 0 | 0 |
| PROST2012007 | 0 | 0 | 0 | 0 | 0 | 7.864 | 0 |
| PROST2015710 | 0 | 0 | 0 | 0 | 0 | 42.668 | 0 |
| PROST2017203 | 0 | 0 | 0 | 7.524 | 0 | 0 | 0 |
| PROST2017692 | 0 | 0 | 0 | 0 | 0 | 2.553 | 0 |
| PUAEN1000057 | 0 | 0 | 0 | 11.896 | 0 | 0 | 0 |
| PUAEN1000065 | 0 | 0 | 0 | 11.643 | 0 | 0 | 0 |
| PUAEN1000161 | 0 | 0 | 0 | 0 | 0 | 19.985 | 0 |
| PUAEN1000322 | 9.325 | 0 | 0 | 0 | 0 | 4.429 | 0 |
| PUAEN2000374 | 2.335 | 0 | 0 | 0 | 0 | 1.109 | 0 |
| PUAEN2000497 | 0 | 0 | 0 | 0 | 0 | 8.932 | 0 |
| PUAEN2000535 | 0 | 0 | 0 | 0 | 0 | 2.786 | 0 |
| PUAEN2001882 | 0 | 0 | 0 | 0 | 0 | 38.043 | 0 |
| PUAEN2007044 | 0 | 0 | 0 | 8.305 | 0 | 0 | 0 |
| SALGL1000157 | 0 | 0 | 0 | 1.507 | 0 | 0 | 0 |
| SKMUS1000138 | 0 | 0 | 56.007 | 0 | 0 | 0 | 0 |
| SKMUS2000020 | 0 | 0 | 0 | 21.341 | 0 | 0 | 0 |
| SKMUS2000780 | 0 | 0 | 0 | 0 | 0 | 32.962 | 0 |
| SKMUS2000931 | 0 | 0 | 0 | 0 | 0 | 3.771 | 0 |
| SKMUS2000933 | 0 | 0 | 0 | 35.951 | 0 | 0 | 0 |
| SKMUS2001454 | 0 | 29.449 | 0 | 0 | 0 | 1.169 | 0 |
| SKNMC1000137 | 0 | 0 | 0 | 24.569 | 0 | 0 | 0 |
| SKNMC2000097 | 6.502 | 38.890 | 0 | 2.583 | 0 | 3.088 | 0 |
| SKNMC2000635 | 0 | 0 | 0 | 2.079 | 0 | 5.593 | 0 |
| SKNMC2000649 | 0 | 0 | 0 | 0 | 0 | 6.322 | 0 |
| SKNMC2000698 | 0 | 0 | 0 | 0 | 0 | 7.156 | 0 |
| SKNMC2000877 | 0 | 0 | 0 | 0 | 0 | 8.211 | 0 |
| SKNMC2001324 | 0 | 0 | 0 | 37.417 | 0 | 0 | 0 |
| SKNMC2001503 | 0 | 0 | 0 | 0 | 0 | 10.744 | 0 |
| SKNSH1000101 | 0 | 56.047 | 0 | 0 | 0 | 0 | 0 |
| SKNSH2000319 | 0 | 0 | 0 | 0 | 0 | 24.846 | 0 |
| SKNSH2001875 | 0 | 0 | 0 | 37.625 | 0 | 0 | 0 |
| SKNSH2002325 | 0 | 0 | 0 | 0 | 0 | 13.226 | 0 |
| SKNSH2002768 | 0 | 0 | 0 | 13.719 | 0 | 8.203 | 0 |
| SKNSH2008940 | 0 | 0 | 0 | 0 | 0 | 6.644 | 0 |
| SMINT1000117 | 0 | 0 | 0 | 7.621 | 0 | 0 | 0 |
| SMINT2000441 | 0 | 0 | 0 | 2.615 | 0 | 0 | 0 |
| SMINT2000468 | 0 | 0 | 0 | 0 | 0 | 15.854 | 0 |
| SMINT2000602 | 0 | 0 | 53.428 | 0 | 0 | 0 | 0 |
| SMINT2000659 | 0 | 0 | 0 | 1.610 | 0 | 0 | 0 |
| SMINT2002976 | 0 | 0 | 0 | 30.224 | 0 | 0 | 0 |
| SMINT2003074 | 9.999 | 0 | 0 | 7.943 | 0 | 0 | 0 |
| SMINT2003169 | 0 | 0 | 0 | 8.176 | 0 | 1.630 | 0 |
| SMINT2003340 | 0 | 0 | 0 | 17.024 | 0 | 0 | 0 |
| SMINT2004023 | 0 | 0 | 0 | 8.837 | 0 | 0 | 0 |
| SMINT2004144 | 0 | 0 | 0 | 0 | 0 | 0 | 9.901 |
| SMINT2004872 | 0 | 0 | 0 | 24.508 | 0 | 0 | 0 |
| SMINT2005368 | 0 | 0 | 0 | 6.805 | 0 | 4.069 | 0 |
| SMINT2005688 | 0 | 0 | 0 | 28.923 | 0 | 0 | 0 |
| SMINT2008960 | 4.990 | 0 | 0 | 3.964 | 0 | 0 | 0 |
| SMINT2011567 | 0 | 0 | 0 | 0 | 0 | 32.872 | 0 |
| SPLEN1000106 | 0 | 0 | 0 | 0 | 0 | 8.064 | 0 |
| SPLEN1000116 | 0 | 0 | 0 | 5.735 | 0 | 1.715 | 0 |
| SPLEN2000210 | 0 | 0 | 0 | 76.909 | 0 | 0 | 0 |
| SPLEN2000255 | 0 | 0 | 0 | 8.899 | 0 | 0 | 0 |
| SPLEN2000310 | 0 | 0 | 0 | 76.909 | 0 | 0 | 0 |
| SPLEN2000348 | 0 | 0 | 0 | 16.209 | 0 | 0 | 0 |
| SPLEN2000414 | 0 | 0 | 0 | 76.909 | 0 | 0 | 0 |
| SPLEN2000505 | 0 | 0 | 0 | 2.442 | 0 | 0 | 0 |
| SPLEN2000874 | 0 | 0 | 47.377 | 0 | 0 | 0 | 10.632 |
| SPLEN2002477 | 0 | 0 | 0 | 9.415 | 0 | 0 | 0 |
| SPLEN2004555 | 0 | 0 | 0 | 0 | 0 | 3.688 | 0 |
| SPLEN2006122 | 0 | 0 | 0 | 0 | 0 | 2.918 | 0 |
| SPLEN2006133 | 0 | 0 | 0 | 5.497 | 0 | 0 | 0 |
| SPLEN2006374 | 0 | 0 | 0 | 17.614 | 0 | 0 | 0 |
| SPLEN2008496 | 0 | 0 | 0 | 0 | 0 | 26.824 | 0 |
| SPLEN2009512 | 0 | 0 | 0 | 0 | 0 | 57.379 | 0 |
| SPLEN2010195 | 0 | 0 | 0 | 0 | 34.507 | 0 | 0 |
| SPLEN2015310 | 4.181 | 50.011 | 0 | 0 | 0 | 0 | 0 |
| SPLEN2017620 | 0 | 0 | 0 | 0 | 0 | 53.276 | 0 |
| SPLEN2017918 | 0 | 0 | 0 | 0 | 0 | 3.159 | 0 |
| SPLEN2019405 | 0 | 0 | 0 | 8.298 | 0 | 0 | 0 |
| SPLEN2019446 | 0 | 0 | 0 | 60.294 | 0 | 0 | 0 |
| SPLEN2019774 | 0 | 0 | 0 | 51.478 | 0 | 0 | 0 |
| SPLEN2031424 | 0 | 0 | 0 | 44.123 | 0 | 0 | 0 |
| SPLEN2031547 | 0 | 0 | 0 | 0 | 0 | 5.455 | 0 |
| SPLEN2032157 | 0 | 0 | 0 | 33.110 | 0 | 0 | 0 |
| SPLEN2032356 | 0 | 0 | 0 | 0 | 0 | 66.573 | 0 |
| SPLEN2039379 | 0 | 0 | 0 | 16.781 | 0 | 0 | 0 |
| SPLEN2042535 | 0 | 0 | 0 | 0 | 0 | 24.667 | 0 |
| STOMA1000047 | 0 | 0 | 0 | 18.360 | 0 | 0 | 0 |
| STOMA2006780 | 0 | 0 | 0 | 0 | 0 | 30.095 | 0 |
| SYNOV2001144 | 0 | 0 | 0 | 0 | 0 | 4.900 | 0 |
| SYNOV2001239 | 0 | 0 | 0 | 5.454 | 0 | 0 | 0 |
| SYNOV2001374 | 0 | 0 | 0 | 10.613 | 0 | 0 | 0 |
| SYNOV2009172 | 0 | 45.405 | 0 | 3.015 | 0 | 0 | 0 |
| TBAES2009387 | 0 | 0 | 0 | 3.097 | 0 | 0 | 0 |
| TESTI1000101 | 0 | 0 | 0 | 0 | 0 | 29.680 | 0 |
| TESTI2001099 | 0 | 0 | 0 | 0 | 0 | 1.698 | 0 |
| TESTI2001153 | 0 | 0 | 0 | 2.307 | 0 | 0 | 0 |
| TESTI2001269 | 0 | 0 | 0 | 0 | 0 | 5.674 | 0 |
| TESTI2001829 | 0 | 0 | 0 | 4.920 | 0 | 0 | 0 |
| TESTI2002149 | 0 | 0 | 0 | 0 | 0 | 5.795 | 0 |
| TESTI2003071 | 0 | 0 | 0 | 0 | 0 | 12.088 | 0 |
| TESTI2004031 | 2.924 | 0 | 0 | 2.323 | 0 | 2.778 | 0 |
| TESTI2004737 | 0 | 0 | 0 | 10.264 | 0 | 0 | 0 |
| TESTI2006051 | 0 | 0 | 41.159 | 0 | 0 | 0 | 0 |
| TESTI2006588 | 0 | 0 | 0 | 6.274 | 0 | 0 | 0 |
| TESTI2006872 | 0 | 0 | 0 | 0 | 0 | 19.038 | 0 |
| TESTI2007311 | 0 | 0 | 0 | 0 | 0 | 13.329 | 0 |
| TESTI2007998 | 0 | 0 | 0 | 0 | 0 | 5.478 | 0 |
| TESTI2008240 | 1.824 | 0 | 0 | 5.796 | 0 | 1.733 | 0 |
| TESTI2009577 | 0 | 0 | 0 | 0 | 0 | 6.123 | 0 |
| TESTI2009987 | 0 | 0 | 0 | 0 | 0 | 17.673 | 0 |
| TESTI2012778 | 2.696 | 0 | 0 | 0 | 0 | 1.920 | 0 |
| TESTI2013295 | 0 | 0 | 0 | 0 | 0 | 9.228 | 0 |
| TESTI2015213 | 3.744 | 0 | 0 | 0 | 0 | 1.778 | 0 |
| TESTI2017954 | 0 | 0 | 0 | 0 | 0 | 2.295 | 0 |
| TESTI2025422 | 0 | 0 | 0 | 28.764 | 0 | 0 | 0 |
| TESTI2026525 | 0 | 0 | 0 | 0 | 0 | 15.074 | 0 |
| TESTI2026647 | 0 | 0 | 0 | 68.465 | 0 | 0 | 0 |
| TESTI2027271 | 0 | 0 | 0 | 30.596 | 0 | 0 | 0 |
| TESTI2033441 | 0 | 0 | 0 | 6.358 | 0 | 0 | 0 |
| TESTI2046456 | 0 | 0 | 0 | 0 | 0 | 3.207 | 0 |
| TESTI4002799 | 0 | 0 | 0 | 0 | 0 | 1.530 | 0 |
| TESTI4013441 | 0 | 0 | 0 | 0.909 | 0 | 2.174 | 0 |
| TESTI4037156 | 0 | 0 | 0 | 0 | 0 | 1.917 | 0 |
| TESTI4043140 | 0 | 0 | 0 | 16.066 | 0 | 19.213 | 0 |
| THYMU1000002 | 0 | 0 | 0 | 0 | 0 | 2.051 | 0 |
| THYMU1000016 | 0 | 0 | 0 | 0 | 0 | 1.339 | 0 |
| THYMU1000374 | 0 | 0 | 0 | 8.679 | 0 | 0 | 0 |
| THYMU1000428 | 0 | 49.596 | 0 | 6.587 | 0 | 0 | 0 |
| THYMU2000382 | 0 | 0 | 0 | 0 | 0 | 4.200 | 0 |
| THYMU2000436 | 0 | 0 | 0 | 5.339 | 0 | 0 | 0 |
| THYMU2002023 | 0 | 0 | 0 | 77.651 | 0 | 0 | 0 |
| THYMU2002583 | 0 | 0 | 0 | 0 | 0 | 1.796 | 0 |
| THYMU2003419 | 0 | 0 | 0 | 2.910 | 0 | 0 | 0 |
| THYMU2003440 | 0 | 0 | 0 | 0 | 0 | 80.601 | 0 |
| THYMU2003542 | 0 | 0 | 0 | 12.522 | 0 | 0 | 0 |
| THYMU2003891 | 0 | 25.462 | 0 | 0 | 0 | 0 | 0 |
| THYMU2004344 | 0 | 0 | 0 | 7.402 | 0 | 0 | 0 |
| THYMU2005546 | 0 | 0 | 0 | 0 | 48.649 | 1.895 | 0 |
| THYMU2009658 | 2.555 | 30.564 | 0 | 4.059 | 0 | 1.214 | 0 |
| THYMU2010161 | 0 | 0 | 0 | 87.420 | 0 | 0 | 0 |
| THYMU2013136 | 0 | 0 | 0 | 0 | 0 | 2.834 | 0 |
| THYMU2013916 | 0 | 0 | 0 | 0 | 0 | 3.843 | 0 |
| THYMU2014067 | 0 | 0 | 0 | 87.420 | 0 | 0 | 0 |
| THYMU2015762 | 7.231 | 0 | 0 | 0 | 0 | 0 | 43.251 |
| THYMU2018126 | 0 | 63.479 | 0 | 0 | 0 | 0 | 0 |
| THYMU2033308 | 0 | 0 | 0 | 0 | 0 | 48.382 | 0 |
| THYMU2035735 | 0 | 0 | 0 | 0 | 0 | 4.571 | 0 |
| THYMU2039334 | 0 | 99.053 | 0 | 0 | 0 | 0 | 0 |
| THYMU3034671 | 0 | 0 | 0 | 13.819 | 0 | 0 | 0 |
| TKIDN1000001 | 0 | 0 | 0 | 0 | 0 | 38.560 | 0 |
| TKIDN2002738 | 0 | 0 | 40.764 | 0 | 0 | 0 | 0 |
| TKIDN2015423 | 0 | 0 | 0 | 0 | 0 | 2.206 | 0 |
| TLIVE2002046 | 0 | 0 | 0 | 10.517 | 0 | 0 | 0 |
| TRACH1000063 | 0 | 0 | 0 | 12.462 | 0 | 0 | 0 |
| TRACH1000181 | 0 | 0 | 0 | 0 | 0 | 16.414 | 0 |
| TRACH1000205 | 0 | 0 | 0 | 0 | 0 | 13.635 | 0 |
| TRACH2000079 | 3.951 | 0 | 0 | 3.139 | 0 | 9.384 | 0 |
| TRACH2000248 | 0 | 0 | 0 | 8.085 | 0 | 0 | 0 |
| TRACH2000321 | 0 | 0 | 0 | 5.694 | 0 | 1.702 | 0 |
| TRACH2000472 | 0 | 0 | 0 | 4.948 | 0 | 0 | 0 |
| TRACH2000972 | 0 | 0 | 0 | 10.302 | 0 | 0 | 0 |
| TRACH2001289 | 0 | 0 | 0 | 1.969 | 0 | 1.178 | 0 |
| TRACH2001432 | 0 | 0 | 0 | 0 | 0 | 11.562 | 0 |
| TRACH2001463 | 0 | 0 | 0 | 0 | 0 | 10.608 | 0 |
| TRACH2001523 | 0 | 0 | 0 | 9.851 | 0 | 5.890 | 0 |
| TRACH2001607 | 0 | 0 | 0 | 0 | 0 | 3.480 | 0 |
| TRACH2002092 | 0 | 0 | 0 | 16.265 | 0 | 0 | 0 |
| TRACH2002333 | 0 | 0 | 0 | 0 | 0 | 50.930 | 0 |
| TRACH2002537 | 0 | 0 | 0 | 0 | 0 | 3.213 | 0 |
| TRACH2003323 | 0 | 0 | 0 | 10.312 | 0 | 0 | 0 |
| TRACH2004499 | 0 | 0 | 0 | 0 | 0 | 10.181 | 0 |
| TRACH2007399 | 0 | 0 | 0 | 2.729 | 41.897 | 1.632 | 0 |
| TRACH2007969 | 0 | 0 | 0 | 29.409 | 0 | 0 | 0 |
| TRACH2012497 | 0 | 0 | 0 | 75.015 | 0 | 0 | 0 |
| TRACH2014077 | 0 | 0 | 0 | 0 | 0 | 11.785 | 0 |
| TRACH2014442 | 0 | 0 | 0 | 0 | 0 | 15.600 | 0 |
| TRACH2015823 | 0 | 0 | 0 | 0 | 0 | 7.579 | 0 |
| TRACH2015824 | 0 | 0 | 0 | 26.932 | 0 | 0 | 0 |
| TRACH2016835 | 0 | 0 | 0 | 0 | 0 | 63.939 | 0 |
| TRACH2018317 | 0 | 0 | 0 | 0 | 0 | 20.300 | 0 |
| TRACH2018446 | 0 | 0 | 0 | 0 | 0 | 14.489 | 0 |
| TRACH2025535 | 0 | 0 | 0 | 3.364 | 0 | 0 | 0 |
| TRACH3000692 | 0 | 0 | 0 | 0 | 0 | 16.238 | 0 |
| TRACH3004721 | 0 | 0 | 0 | 0 | 0 | 20.144 | 0 |
| TRACH3007625 | 8.574 | 0 | 0 | 6.811 | 0 | 4.073 | 0 |
| UMVEN2000133 | 0 | 0 | 0 | 0 | 0 | 2.031 | 0 |
| UMVEN2000354 | 0 | 0 | 0 | 0 | 0 | 5.708 | 0 |
| UMVEN2000453 | 0 | 0 | 0 | 3.025 | 0 | 0 | 0 |
| UTERU2000095 | 0 | 0 | 0 | 0 | 36.813 | 0 | 0 |
| UTERU2000263 | 0 | 0 | 0 | 0 | 0 | 3.550 | 0 |
| UTERU2000424 | 0 | 0 | 0 | 4.921 | 0 | 2.943 | 0 |
| UTERU2000541 | 0 | 0 | 0 | 36.544 | 0 | 0 | 0 |
| UTERU2001607 | 0 | 0 | 0 | 0 | 0 | 5.111 | 0 |
| UTERU2002662 | 0 | 0 | 0 | 0 | 0 | 6.164 | 0 |
| UTERU2004037 | 15.993 | 0 | 0 | 12.704 | 0 | 0 | 0 |
| UTERU2004197 | 0 | 0 | 0 | 0 | 0 | 6.967 | 0 |
| UTERU2005593 | 0 | 0 | 0 | 0 | 0 | 7.777 | 0 |
| UTERU2005621 | 0 | 0 | 0 | 0 | 0 | 7.810 | 0 |
| UTERU2007724 | 0 | 0 | 0 | 38.068 | 0 | 0 | 0 |
| UTERU2008085 | 1.976 | 0 | 0 | 1.570 | 0 | 0.313 | 1.970 |
| UTERU2010417 | 0 | 27.509 | 0 | 0 | 0 | 6.554 | 0 |
| UTERU2012252 | 0 | 0 | 0 | 0 | 0 | 74.391 | 0 |
| UTERU2013586 | 0 | 0 | 0 | 0 | 0 | 22.827 | 0 |
| UTERU2015062 | 0 | 0 | 0 | 82.930 | 0 | 0 | 0 |
| UTERU2015108 | 0 | 0 | 0 | 0 | 0 | 21.862 | 0 |
| UTERU2015653 | 0 | 0 | 0 | 0 | 51.890 | 2.021 | 0 |
| UTERU2024969 | 0 | 0 | 0 | 0 | 0 | 11.126 | 0 |

Genes whose expression levels were negligibly low in each of the above-described libraries are shown with their clone names below.

3NB691000085, 3NB691000113, 3NB691000116, 3NB691000129, 3NB691000173, 3NB691000191, 3NB692000012, 3NB692000029, 3NB692000102, 3NB692000154, 3NB692000276, 3NB692000305, 3NB692000429, 3NB692000484, 3NB692000529, 3NB692000545, 3NB692000973, 3NB692001002, 3NB692001034, 3NB692001040, 3NB692001123, 3NB692001267, 3NB692001288, 3NB692001334, 3NB692001339, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001520, 3NB692001528, 3NB692001548, 3NB692001557, 3NB692001637, 3NB692001719, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, BGGI11000123, BGGI11000193, BGGI11000285, BGGI12000022, BGGI12000067, BGGI12000161, BGGI12000533, BGGI12000544, BGGI12000684, BGGI12000693, BGGI12000839, BGGI12001002, BGGI12001075, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000137, BNGH41000153, BNGH41000169, BNGH41000177, BNGH41000216, BNGH41000235, BNGH42000130, BNGH42000132, BNGH42000405, BNGH42000532, BNGH42000675, BNGH42000739, BNGH42000791, BNGH42000815, BNGH42000994, BNGH42001247, BNGH42001406, BNGH42001576, BNGH42001724, BNGH42002168, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42005968, BNGH42006135, BNGH42006226, BNGH42006234, BNGH42007037, BNGH42007366, BNGH42007594, BNGH42007798, BNGH42008199, BNGH42008510, BNGH42008603, BNGH42008649, BNGH42008743, BNGH42008832, BNGH42008850, BNGH42008943, BNGH42009021, BNGH42009025, BRACE1000020, BRACE1000042, BRACE1000047, BRACE1000051, BRACE1000065, BRACE1000070, BRACE1000073, BRACE1000084, BRACE1000087, BRACE1000092, BRACE1000093, BRACE1000101, BRACE1000114, BRACE1000115, BRACE1000124, BRACE1000131, BRACE1000159, BRACE1000166, BRACE1000169, BRACE1000186, BRACE1000187, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000183, BRACE2000229, BRACE2000232, BRACE2000245, BRACE2000256, BRACE2000266, BRACE2000267, BRACE2000280, BRACE2000300, BRACE2000331, BRACE2000332, BRACE2000347, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000753, BRACE2000815, BRACE2000864, BRACE2000885, BRACE2000893, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001117, BRACE2001154, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001497, BRACE2001508, BRACE2001534, BRACE2001543, BRACE2001564, BRACE2001588, BRACE2001607, BRACE2001626, BRACE2001673, BRACE2001677, BRACE2001705, BRACE2001709, BRACE2001711, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001892, BRACE2001898, BRACE2001923, BRACE2001942, BRACE2001944, BRACE2001954, BRACE2001971, BRACE2001972, BRACE2001973, BRACE2002026, BRACE2002034, BRACE2002050, BRACE2002081, BRACE2002091, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002661, BRACE2002676, BRACE2002677, BRACE2002682, BRACE2002685, BRACE2002695, BRACE2002707, BRACE2002736, BRACE2002755, BRACE2002762, BRACE2002772, BRACE2002792, BRACE2002796, BRACE2002803, BRACE2002807, BRACE2002812, BRACE2002834, BRACE2002849, BRACE2002857, BRACE2002861, BRACE2002884, BRACE2002896, BRACE2002948, BRACE2003037, BRACE2003078, BRACE2003097, BRACE2003110, BRACE2003168, BRACE2003172, BRACE2003199, BRACE2003210, BRACE2003269, BRACE2003285, BRACE2003304, BRACE2003319, BRACE2003398, BRACE2003420, BRACE2003428, BRACE2003431, BRACE2003512, BRACE2003516, BRACE2003527, BRACE2003539, BRACE2003540, BRACE2003594, BRACE2003598, BRACE2003609, BRACE2003614, BRACE2003639, BRACE2003700, BRACE2003713, BRACE2003766, BRACE2003788, BRACE2003800, BRACE2003802, BRACE2003825, BRACE2003845, BRACE2003846, BRACE2003847, BRACE2003848, BRACE2003873, BRACE2003885, BRACE2003887, BRACE2003892, BRACE2003904, BRACE2003905, BRACE2003928, BRACE2003944, BRACE2003954, BRACE2003982, BRACE2003987, BRACE2005034, BRACE2005047, BRACE2005063, BRACE2005083, BRACE2005087, BRACE2005090, BRACE2005099, BRACE2005118, BRACE2005124, BRACE2005138, BRACE2005160, BRACE2005169, BRACE2005189, BRACE2005193, BRACE2005196, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005433, BRACE2005448, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006055, BRACE2006072, BRACE2006075, BRACE2006084, BRACE2006089, BRACE2006102, BRACE2006105, BRACE2006122, BRACE2006137, BRACE2006143, BRACE2006162, BRACE2006167, BRACE2006174, BRACE2006240, BRACE2006245, BRACE2006249, BRACE2006258, BRACE2006264, BRACE2006274, BRACE2006281, BRACE2006298, BRACE2006319, BRACE2006344, BRACE2006354, BRACE2006363, BRACE2006378, BRACE2006380, BRACE2006393, BRACE2006397, BRACE2006413, BRACE2006417, BRACE2006453, BRACE2006459, BRACE2006488, BRACE2006514, BRACE2006534, BRACE2006540, BRACE2006547, BRACE2006564, BRACE2006587, BRACE2006598, BRACE2006636, BRACE2006685, BRACE2006703, BRACE2006743, BRACE2006753, BRACE2006783, BRACE2006821, BRACE2006833, BRACE2006859, BRACE2006900, BRACE2006909, BRACE2006911, BRACE2006924, BRACE2006935, BRACE2006937, BRACE2006944, BRACE2006951, BRACE2006982, BRACE2006986, BRACE2007013, BRACE2007064, BRACE2007068, BRACE2007087, BRACE2007109, BRACE2007138, BRACE2007174, BRACE2007197, BRACE2007201, BRACE2007203, BRACE2007232, BRACE2007254, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007518, BRACE2007522, BRACE2007527, BRACE2007538, BRACE2007563, BRACE2007567, BRACE2007577, BRACE2007621, BRACE2007640, BRACE2007641, BRACE2007646, BRACE2007663, BRACE2007682, BRACE2007685, BRACE2007727, BRACE2007749, BRACE2007760, BRACE2007761, BRACE2007764, BRACE2007767, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008358, BRACE2008361, BRACE2008380, BRACE2008385, BRACE2008401, BRACE2008410, BRACE2008420, BRACE2008443, BRACE2008473, BRACE2008480, BRACE2008481, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008708, BRACE2008710, BRACE2008754, BRACE2008797, BRACE2008847, BRACE2008857, BRACE2008861, BRACE2008867, BRACE2008883, BRACE2008917, BRACE2008919, BRACE2008941, BRACE2008948, BRACE2008960, BRACE2008999, BRACE2009014, BRACE2009016, BRACE2009031, BRACE2009037, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009533, BRACE2009558, BRACE2009566, BRACE2009591, BRACE2009620, BRACE2009654, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317, BRACE2012510, BRACE2012528, BRACE2012625, BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2014108, BRACE2014161, BRACE2014232, BRACE2014257, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015436, BRACE2015741, BRACE2015756, BRACE2015969, BRACE2015995, BRACE2016058, BRACE2016194, BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017397, BRACE2017410, BRACE2017438, BRACE2017574, BRACE2017580, BRACE2017587, BRACE2017720, BRACE2017844, BRACE2017872, BRACE2017929, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021014, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004150, BRACE3004205, BRACE3004358, BRACE3004371, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005938, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010428, BRACE3010435, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000354, BRAMY2000388, BRAMY2000411, BRAMY2000508, BRAMY2000562, BRAMY2000585, BRAMY2000653, BRAMY2000814, BRAMY2000946, BRAMY2000981, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004387, BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016325, BRAMY2016327, BRAMY2016386, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019600, BRAMY2019643, BRAMY2019710, BRAMY2019963, BRAMY2019985, BRAMY2019989, BRAMY2020050, BRAMY2020058, BRAMY2020106, BRAMY2020270, BRAMY2020354, BRAMY2020355, BRAMY2020412, BRAMY2020427, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2023939, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027739, BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028783, BRAMY2028796, BRAMY2028856, BRAMY2028914, BRAMY2029204, BRAMY2029602, BRAMY2029988, BRAMY2030098, BRAMY2030109, BRAMY2030206, BRAMY2030477, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3003026, BRAMY3003109, BRAMY3003205, BRAMY3003310, BRAMY3003566, BRAMY3003723, BRAMY3004126, BRAMY3004224, BRAMY3004364, BRAMY3004377, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010492, BRAMY3010603, BRAMY3010654, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000007, BRAWH1000037, BRAWH1000045, BRAWH1000093, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417, BRAWH2000443, BRAWH2000471, BRAWH2000476, BRAWH2000482, BRAWH2000488, BRAWH2000494, BRAWH2000500, BRAWH2000503, BRAWH2000522, BRAWH2000554, BRAWH2000595, BRAWH2000633, BRAWH2000636, BRAWH2000643, BRAWH2000651, BRAWH2000682, BRAWH2000686, BRAWH2000752, BRAWH2000859, BRAWH2000866, BRAWH2000892, BRAWH2000901, BRAWH2000926, BRAWH2000944, BRAWH2000984, BRAWH2001019, BRAWH2001037, BRAWH2001052, BRAWH2001103, BRAWH2001129, BRAWH2001137, BRAWH2001141, BRAWH2001159, BRAWH2001166, BRAWH2001171, BRAWH2001186, BRAWH2001203, BRAWH2001238, BRAWH2001239, BRAWH2001255, BRAWH2001321, BRAWH2001355, BRAWH2001378, BRAWH2001395, BRAWH2001406, BRAWH2001412, BRAWH2001418, BRAWH2001424, BRAWH2001436, BRAWH2001438, BRAWH2001439, BRAWH2001459, BRAWH2001461, BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003025, BRAWH2003135, BRAWH2003240, BRAWH2003307, BRAWH2003355, BRAWH2003366, BRAWH2003662, BRAWH2003689, BRAWH2003693, BRAWH2003818, BRAWH2003832, BRAWH2003948, BRAWH2003964, BRAWH2004044, BRAWH2004068, BRAWH2004095, BRAWH2004136, BRAWH2004217, BRAWH2004580, BRAWH2004731, BRAWH2004779, BRAWH2004842, BRAWH2004884, BRAWH2004923, BRAWH2005008, BRAWH2005068, BRAWH2005074, BRAWH2005225, BRAWH2005315, BRAWH2005517, BRAWH2005524, BRAWH2005533, BRAWH2005578, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2006996, BRAWH2007069, BRAWH2007308, BRAWH2007406, BRAWH2007550, BRAWH2007570, BRAWH2007591, BRAWH2007605, BRAWH2007658, BRAWH2007664, BRAWH2007800, BRAWH2007808, BRAWH2007825, BRAWH2007862, BRAWH2007890, BRAWH2008058, BRAWH2008128, BRAWH2008219, BRAWH2008255, BRAWH2008292, BRAWH2008297, BRAWH2008366, BRAWH2008380, BRAWH2008540, BRAWH2008577, BRAWH2008706, BRAWH2008790, BRAWH2008903, BRAWH2008956, BRAWH2008993, BRAWH2009112, BRAWH2009238, BRAWH2009261, BRAWH2009263, BRAWH2009298, BRAWH2009304, BRAWH2009307, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014188, BRAWH2014234, BRAWH2014379, BRAWH2014414, BRAWH2014473, BRAWH2014511, BRAWH2014645, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017407, BRAWH2017433, BRAWH2017523, BRAWH2017534, BRAWH2017542, BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018729, BRAWH2018745, BRAWH2018875, BRAWH2019053, BRAWH2019055, BRAWH2019198, BRAWH3000078, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002600, BRAWH3002819, BRAWH3003019, BRAWH3003136, BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021545, BRAWH3021574, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN1000168, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2009432, BRCAN2010374, BRCAN2010376, BRCAN2010547, BRCAN2010581, BRCAN2010665, BRCAN2011254, BRCAN2011316, BRCAN2011602, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028040, BRCAN2028319, BRCAN2028338, BRCAN2028355, BRCAN2028378, BRCAN2028460, BRCAN2028545, BRCAN2028634, BRCAN2028702, BRCOC1000040, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003187, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010123, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019910, BRCOC2019934, BRCOC2020122, BRCOC2020142, BRHIP1000058, BRHIP1000072, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000621, BRHIP2000691, BRHIP2000819, BRHIP2000826, BRHIP2000920, BRHIP2001074, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007305, BRHIP2007307, BRHIP2007616, BRHIP2007690, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011491, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP201438.6, BRHIP2014387, BRHIP2014954, BRHIP2015153, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020799, BRHIP2020842, BRHIP2020859, BRHIP2020930, BRHIP2021102, BRHIP2021289, BRHIP2021495, BRHIP2021615, BRHIP2021744, BRHIP2021762, BRHIP2021858, BRHIP2021870, BRHIP2021929, BRHIP2022006, BRHIP2022054, BRHIP2022221, BRHIP2022228, BRHIP2022298, BRHIP2022326, BRHIP2022467, BRHIP2022708, BRHIP2022986, BRHIP2023071, BRHIP2023229, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024398, BRHIP2024549, BRHIP2024742, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000017, BRHIP3000060, BRHIP3000070, BRHIP3000081, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007223, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3018784, BRHIP3019643, BRHIP3019824, BRHIP3019880, BRHIP3019956, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000498, BRSSN2000518, BRSSN2000561, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009518, BRSSN2009627, BRSSN2009630, BRSSN2009702, BRSSN2010019, BRSSN2010110, BRSSN2010587, BRSSN2010596, BRSSN2010830, BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014424, BRSSN2014513, BRSSN2014556, BRSSN2014573, BRSSN2014610, BRSSN2014636, BRSSN2014685, BRSSN2015199, BRSSN2015238, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000083, BRSTN1000097, BRSTN2000058, BRSTN2000070, BRSTN2000220, BRSTN2000312, BRSTN2000367, BRSTN2000536, BRSTN2000872, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012174, BRSTN2012284, BRSTN2012320, BRSTN2012380, BRSTN2013354, BRSTN2013502, BRSTN2014490, BRSTN2014633, BRSTN2014751, BRSTN2014770, BRSTN2015015, BRSTN2015889, BRSTN2015978, BRSTN2016066, BRSTN2016335, BRSTN2016678, BRSTN2016892, BRSTN2016918, BRSTN2016992, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2007998, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562, BRTHA2012714, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016577, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027229, BRTHA2027250, BRTHA2027546, BRTHA2028297, BRTHA2029969, BRTHA20.30036, BRTHA2030213, BRTHA2031517, BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003074, BRTHA3003225, BRTHA3003339, BRTHA3003343, BRTHA3003417, BRTHA3003449, BRTHA3003474, BRTHA3003704, BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011194, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3021971, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, CHONS2002829, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000009, CTONG1000010, CTONG1000020, CTONG1000022, CTONG1000040, CTONG1000052, CTONG1000056, CTONG1000062, CTONG1000086, CTONG1000088, CTONG1000093, CTONG1000097, CTONG1000113, CTONG1000137, CTONG1000165, CTONG1000175, CTONG1000180, CTONG1000181, CTONG1000218, CTONG1000220, CTONG1000241, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042, CTONG2000063, CTONG2000201, CTONG2000218, CTONG2000279, CTONG2000303, CTONG2000330, CTONG2000395, CTONG2000411, CTONG2000452, CTONG2000465, CTONG2000480, CTONG2000508, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001366, CTONG2001413, CTONG2001428, CTONG2001513, CTONG2001524, CTONG2001533, CTONG2001598, CTONG2001681, CTONG2001724, CTONG2001748, CTONG2001749, CTONG2001800, CTONG2001820, CTONG2001858, CTONG2001877, CTONG2001911, CTONG2001932, CTONG2001955, CTONG2002066, CTONG2002073, CTONG2002095, CTONG2002143, CTONG2002270, CTONG2002395, CTONG2002417, CTONG2002418, CTONG2002453, CTONG2002520, CTONG2002558, CTONG2002590, CTONG2002709, CTONG2002721, CTONG2002744, CTONG2002766, CTONG2002803, CTONG2002816, CTONG2002820, CTONG2002832, CTONG2002841, CTONG2002965, CTONG2002999, CTONG2003028, CTONG2003100, CTONG2003115, CTONG2003189, CTONG2003245, CTONG2003293, CTONG2003348, CTONG2003350, CTONG2003361, CTONG2003372, CTONG2003375, CTONG2003427, CTONG2003517, CTONG2003524, CTONG2003599, CTONG2003680, CTONG2003699, CTONG2003782, CTONG2003889, CTONG2003937, CTONG2003945, CTONG2004000, CTONG2004062, CTONG2004126, CTONG2004264, CTONG2004305, CTONG2004397, CTONG2004423, CTONG2004454, CTONG2004487, CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005290, CTONG2005374, CTONG2005399, CTONG2005468, CTONG2005567, CTONG2005585, CTONG2005615, CTONG2005635, CTONG2005775, CTONG2005795, CTONG2005890, CTONG2005904, CTONG2005913, CTONG2006004, CTONG2006129, CTONG2006223, CTONG2006235, CTONG2006273, CTONG2006310, CTONG2006377, CTONG2006393, CTONG2006412, CTONG2006449, CTONG2006515, CTONG2006524, CTONG2006562, CTONG2006568, CTONG2006611, CTONG2006620, CTONG2006666, CTONG2006691, CTONG2006709, CTONG2006798, CTONG2006836, CTONG2006942, CTONG2006964, CTONG2007007, CTONG2007009, CTONG2007072, CTONG2007078, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007500, CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008321, CTONG2008343, CTONG2008398, CTONG2008402, CTONG2008405, CTONG2008466, CTONG2008518, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010408, CTONG2010508, CTONG2010566, CTONG2010623, CTONG2010652, CTONG2010803, CTONG2010821, CTONG2011188, CTONG2011429, CTONG2011474, CTONG2011676, CTONG2011716, CTONG2011765, CTONG2011779, CTONG2011815, CTONG2011822, CTONG2011825, CTONG2011920, CTONG2011985, CTONG2012001, CTONG2012029, CTONG2012050, CTONG2012077, CTONG2012101, CTONG2012123, CTONG2012158, CTONG2012230, CTONG2012401, CTONG2012422, CTONG2012425, CTONG2012447, CTONG2012473, CTONG2012476, CTONG2012533, CTONG2012554, CTONG2012564, CTONG2012607, CTONG2012661, CTONG2012724, CTONG2012738, CTONG2012745, CTONG2012758, CTONG2012843, CTONG2012847, CTONG2012879, CTONG2012901, CTONG2012996, CTONG2013128, CTONG2013149, CTONG2013156, CTONG2013178, CTONG2013222, CTONG2013284, CTONG2013339, CTONG2013348, CTONG2013352, CTONG2013381, CTONG2013393, CTONG2013511, CTONG2013630, CTONG2013803, CTONG2013907, CTONG2013934, CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014369, CTONG2014389, CTONG2014491, CTONG2014630, CTONG2014635, CTONG2014646, CTONG2014898, CTONG2014946, CTONG2014954, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016056, CTONG2016217, CTONG2016355, CTONG2016408, CTONG2016499, CTONG2016505, CTONG2016559, CTONG2016658, CTONG2016775, CTONG2016797, CTONG2016824, CTONG2016846, CTONG2016869, CTONG2016904, CTONG2016931, CTONG2016942, CTONG2016953, CTONG2017021, CTONG2017094, CTONG2017292, CTONG2017409, CTONG2017429, CTONG2017444, CTONG2017458, CTONG2017500, CTONG2017604, CTONG2017650, CTONG2017798, CTONG2017804, CTONG2017814, CTONG2017910, CTONG2017939, CTONG2017989, CTONG2017998, CTONG2018062, CTONG2018069, CTONG2018147, CTONG2018211, CTONG2018217, CTONG2018279, CTONG2018334, CTONG2018343, CTONG2018379, CTONG2018413, CTONG2018483, CTONG2018629, CTONG2018632, CTONG2018637, CTONG2018652, CTONG2018653, CTONG2018655, CTONG2018735, CTONG2018808, CTONG2018843, CTONG2018900, CTONG2018976, CTONG2019029, CTONG2019063, CTONG2019144, CTONG2019232, CTONG2019248, CTONG2019400, CTONG2019590, CTONG2019652, CTONG2019704, CTONG2019788, CTONG2019822, CTONG2019833, CTONG2020031, CTONG2020108, CTONG2020127, CTONG2020171, CTONG2020374, CTONG2020378, CTONG2020411, CTONG2020445, CTONG2020522, CTONG2020582, CTONG2020638, CTONG2020806, CTONG2020831, CTONG2020974, CTONG2021024, CTONG2021132, CTONG2021168, CTONG2021289, CTONG2022153, CTONG2022601, CTONG2022835, CTONG2023021, CTONG2023449, CTONG2023512, CTONG2024031, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028124, CTONG2028208, CTONG2028309, CTONG2028486, CTONG2028518, CTONG2028687, CTONG2028758, CTONG3000657, CTONG3000686, CTONG3000707, CTONG3000896, CTONG3001123, CTONG3001420, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002674, CTONG3002728, CTONG3002835, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008894, CTONG3008951, CTONG3008959, CTONG3009028, CTONG3009227, CTONG3009287, CTONG3009328, CTONG3009385, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000358, D6OST2000464, D6OST2000507, D9OST2000031, D9OST2000278, D9OST2000440, D9OST2000869, D9OST2001319, D9OST2001433, D9OST2002397, D9OST2002608, D9OST2002673, D9OST2003106, D9OST2003108, D9OST2003137, D9OST2003397, D9OST2003580, D9OST2003594, D9OST2003762, D9OST2003791, D9OST2003989, D9OST2004018, D9OST2004073, D9OST2004417, DFNES1000003, DFNES1000083, DFNES1000107, DFNES1000150, DFNES1000185, DFNES2000011, DFNES2000143, DFNES2000146, DFNES2000212, DFNES2000268, DFNES2000335, DFNES2000426, DFNES2000432, DFNES2000457, DFNES2000471, DFNES2000913, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2002966, DFNES2003081, DFNES2003192, DFNES2003255, DFNES2003742, DFNES2004346, DFNES2004354, DFNES2004383, DFNES2004608, DFNES2004684, DFNES2005266, DFNES2005527, DFNES2005540, DFNES2005690, DFNES2005766, DFNES2005779, DFNES2005875, DFNES2006346, DFNES2006944, DFNES2007113, DFNES2007299, DFNES2007332, DFNES2007634, DFNES2007757, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000024, FCBBF1000027, FCBBF1000038, FCBBF1000053, FCBBF1000061, FCBBF1000069, FCBBF1000077, FCBBF1000115, FCBBF1000117, FCBBF1000118, FCBBF1000121, FCBBF1000125, FCBBF1000131, FCBBF1000139, FCBBF1000155, FCBBF1000157, FCBBF1000171, FCBBF1000174, FCBBF1000182, FCBBF1000197, FCBBF1000198, FCBBF1000206, FCBBF1000220, FCBBF1000232, FCBBF1000233, FCBBF1000243, FCBBF1000270, FCBBF1000272, FCBBF1000280, FCBBF1000294, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000403, FCBBF1000412, FCBBF1000425, FCBBF1000437, FCBBF1000449, FCBBF1000476, FCBBF1000506, FCBBF1000509, FCBBF1000546, FCBBF1000550, FCBBF1000556, FCBBF1000563, FCBBF1000574, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF2000038, FCBBF2000094, FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473, FCBBF2000566, FCBBF2000576, FCBBF2000677, FCBBF2000678, FCBBF2000685, FCBBF2000733, FCBBF2000782, FCBBF2000808, FCBBF2000815, FCBBF2000825, FCBBF2000940, FCBBF2000951, FCBBF2001001, FCBBF2001088, FCBBF2001116, FCBBF2001183, FCBBF2001188, FCBBF2001211, FCBBF2001238, FCBBF2001243, FCBBF2001291, FCBBF2001299, FCBBF2001309, FCBBF2001400, FCBBF2001427, FCBBF2001480, FCBBF2001529, FCBBF2001538, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002349, FCBBF2002370, FCBBF2002541, FCBBF2002626, FCBBF2002801, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005909, FCBBF2005966, FCBBF2006131, FCBBF2006380, FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000110, FCBBF3000115, FCBBF3000120, FCBBF3000168, FCBBF3000175, FCBBF3000227, FCBBF3000228, FCBBF3000229, FCBBF3000233, FCBBF3000269, FCBBF3000361, FCBBF3000367, FCBBF3000388, FCBBF3000434, FCBBF3000462, FCBBF3000473, FCBBF3000501, FCBBF3000518, FCBBF3000536, FCBBF3000550, FCBBF3000847, FCBBF3001018, FCBBF3001020, FCBBF3001081, FCBBF3001235, FCBBF3001281, FCBBF3001302, FCBBF3001377, FCBBF3001470, FCBBF3001594, FCBBF3001657, FCBBF3001818, FCBBF3001855, FCBBF3001890, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004261, FCBBF3004323, FCBBF3004390, FCBBF3004409, FCBBF3004502, FCBBF3004715, FCBBF3004842, FCBBF3004847, FCBBF3004955, FCBBF3005160, FCBBF3005218, FCBBF3005330, FCBBF3005444, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008153, FCBBF3008159, FCBBF3008251, FCBBF3008311, FCBBF3008362, FCBBF3008382, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009541, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016987, FCBBF3017024, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018796, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019714, FCBBF3019716, FCBBF3019784, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078, FCBBF3023368, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028244, FCBBF3028472, FCBBF3028528, FCBBF3028582, FCBBF3028593, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000500, FCBBF4000529, FCBBF4000548, FCBBF5000041, FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000022, FEBRA1000030, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000105, FEBRA2000126, FEBRA2000210, FEBRA2000220, FEBRA2000228, FEBRA2000253, FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000452, FEBRA2000454, FEBRA2000462, FEBRA2000468, FEBRA2000491, FEBRA2000504, FEBRA2000510, FEBRA2000532, FEBRA2000536, FEBRA2000538, FEBRA2000575, FEBRA2000581, FEBRA2000656, FEBRA2000659, FEBRA2000680, FEBRA2000690, FEBRA2000727, FEBRA2000734, FEBRA2000740, FEBRA2000757, FEBRA2000771, FEBRA2000782, FEBRA2000787, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001267, FEBRA2001334, FEBRA2001351, FEBRA2001383, FEBRA2001438, FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001752, FEBRA2001772, FEBRA2001790, FEBRA2001814, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002399, FEBRA2002410, FEBRA2002429, FEBRA2002442, FEBRA2002456, FEBRA2002508, FEBRA2002525, FEBRA2002527, FEBRA2002552, FEBRA2002574, FEBRA2002601, FEBRA2002611, FEBRA2002628, FEBRA2002662, FEBRA2002682, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436, FEBRA2003454, FEBRA2003468, FEBRA2003470, FEBRA2003520, FEBRA2003524, FEBRA2003541, FEBRA2003675, FEBRA2003707, FEBRA2003711, FEBRA2003822, FEBRA2003833, FEBRA2003897, FEBRA2003907, FEBRA2003930, FEBRA2004023, FEBRA2004026, FEBRA2004029, FEBRA2004042, FEBRA2004053, FEBRA2004056, FEBRA2004091, FEBRA2004110, FEBRA2004191, FEBRA2004202, FEBRA2004219, FEBRA2004237, FEBRA2004268, FEBRA2004293, FEBRA2004329, FEBRA2004331, FEBRA2004412, FEBRA2004443, FEBRA2004485, FEBRA2004490, FEBRA2004538, FEBRA2004592, FEBRA2004620, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005427, FEBRA2005458, FEBRA2005476, FEBRA2005572, FEBRA2005701, FEBRA2005726, FEBRA2005734, FEBRA2005752, FEBRA2005778, FEBRA2005787, FEBRA2005788, FEBRA2005995, FEBRA2005998, FEBRA2006055, FEBRA2006061, FEBRA2006112, FEBRA2006150, FEBRA2006165, FEBRA2006255, FEBRA2006270, FEBRA2006315, FEBRA2006346, FEBRA2006354, FEBRA2006357, FEBRA2006368, FEBRA2006370, FEBRA2006379, FEBRA2006396, FEBRA2006409, FEBRA2006418, FEBRA2006427, FEBRA2006476, FEBRA2006491, FEBRA2006519, FEBRA2006521, FEBRA2006553, FEBRA2006627, FEBRA2006664, FEBRA2006667, FEBRA2006726, FEBRA2006736, FEBRA2006793, FEBRA2006808, FEBRA2006817, FEBRA2006890, FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007620, FEBRA2007622, FEBRA2007708, FEBRA2007714, FEBRA2007793, FEBRA2007801, FEBRA2007823, FEBRA2007880, FEBRA2007900, FEBRA2007901, FEBRA2007931, FEBRA2007957, FEBRA2008009, FEBRA2008036, FEBRA2008086, FEBRA2008134, FEBRA2008135, FEBRA2008159, FEBRA2008165, FEBRA2008172, FEBRA2008201, FEBRA2008210, FEBRA2008255, FEBRA2008265, FEBRA2008266, FEBRA2008282, FEBRA2008287, FEBRA2008311, FEBRA2008360, FEBRA2008459, FEBRA2008468, FEBRA2008475, FEBRA2008481, FEBRA2008542, FEBRA2008660, FEBRA2008662, FEBRA2008681, FEBRA2008692, FEBRA2008741, FEBRA2008755, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009419, FEBRA2009437, FEBRA2009478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019711, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022055, FEBRA2022148, FEBRA2022204, FEBRA2022255, FEBRA2022322, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023377, FEBRA2023399, FEBRA2023498, FEBRA2023550, FEBRA2023721, FEBRA2023927, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025427, FEBRA2025463, FEBRA2025477, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEHRT2000325, FEHRT2000364, FEKID1000018, FELIV1000101, FELIV1000137, FELIV1000164, FELNG2000241, HCASM1000021, HCASM1000050, HCASM1000061, HCASM1000115, HCASM1000130, HCASM2000016, HCASM2000138, HCASM2000156, HCASM2000202, HCASM2000214, HCASM2000266, HCASM2000307, HCASM2000363, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCHON1000015, HCHON1000142, HCHON1000166, HCHON1000176, HCHON2000028, HCHON2000038, HCHON2000087, HCHON2000100, HCHON2000156, HCHON2000160, HCHON2000199, HCHON2000212, HCHON2000226, HCHON2000244, HCHON2000265, HCHON2000271, HCHON2000315, HCHON2000322, HCHON2000344, HCHON2000364, HCHON2000395, HCHON2000417, HCHON2000418, HCHON2000473, HCHON2000475, HCHON2000503, HCHON2000508, HCHON2000626, HCHON2000660, HCHON2000663, HCHON2000673, HCHON2000699, HCHON2000705, HCHON2000738, HCHON2000743, HCHON2000751, HCHON2000815, HCHON2000817, HCHON2000818, HCHON2000819, HCHON2000832, HCHON2000851, HCHON2000898, HCHON2000935, HCHON2000942, HCHON2000956, HCHON2000994, HCHON2001084, HCHON2001099, HCHON2001108, HCHON2001116, HCHON2001161, HCHON2001214, HCHON2001233, HCHON2001269, HCHON2001359, HCHON2001450, HCHON2001453, HCHON2001497, HCHON2001505, HCHON2001519, HCHON2001523, HCHON2001535, HCHON2001546, HCHON2001548, HCHON2001577, HCHON2001598, HCHON2001604, HCHON2001607, HCHON2001646, HCHON2001665, HCHON2001712, HCHON2001768, HCHON2001801, HCHON2001853, HCHON2002123, HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004002, HCHON2004007, HCHON2004279, HCHON2004359, HCHON2004531, HCHON2004776, HCHON2004858, HCHON2005048, HCHON2005118, HCHON2005166, HCHON2005802, HCHON2005921, HCHON2005987, HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HCHON2010543, HEART1000010, HEART1000088, HEART1000099, HEART1000102, HEART1000118, HEART1000132, HEART1000139, HEART1000142, HEART1000151, HEART1000173, HEART1000185, HEART2000024, HEART2000035, HEART2000099, HEART2000298, HEART2000306, HEART2000309, HEART2000411, HEART2000418, HEART2000444, HEART2000448, HEART2000492, HEART2000506, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006521, HEART2006557, HEART2006787, HEART2006789, HEART2006909, HEART2007031, HEART2007231, HEART2007443, HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC1000114, HHDPC1000118, HHDPC2000055, HHDPC2000070, HHDPC2000104, HHDPC2000149, HHDPC2000258, HHDPC2000315, HHDPC2000462, HHDPC2000572, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006927, HHDPC2007267, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030, HLUNG1000034, HLUNG1000037, HLUNG1000053, HLUNG1000064, HLUNG1000084, HLUNG1000091, HLUNG1000099, HLUNG1000104, HLUNG1000110, HLUNG1000151, HLUNG1000169, HLUNG2000004, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363, HLUNG2000412, HLUNG2000431, HLUNG2000480, HLUNG2000489, HLUNG2000501, HLUNG2000549, HLUNG2000557, HLUNG2000570, HLUNG2000751, HLUNG2000761, HLUNG2000777, HLUNG2000846, HLUNG2000870, HLUNG2000884, HLUNG2000926, HLUNG2000950, HLUNG2000955, HLUNG2000970, HLUNG2001013, HLUNG2001126, HLUNG2001144, HLUNG2001146, HLUNG2001214, HLUNG2001459, HLUNG2001633, HLUNG2001677, HLUNG2001712, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002465, HLUNG2002562, HLUNG2002659, HLUNG2002707, HLUNG2002811, HLUNG2002942, HLUNG2002949, HLUNG2002958, HLUNG2002982, HLUNG2003003, HLUNG2003042, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004037, HLUNG2004067, HLUNG2004154, HLUNG2004159, HLUNG2004170, HLUNG2004217, HLUNG2004273, HLUNG2004388, HLUNG2004416, HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006812, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008139, HLUNG2008153, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008637, HLUNG2008833, HLUNG2008875, HLUNG2008885, HLUNG2008958, HLUNG2008959, HLUNG2009067, HLUNG2009215, HLUNG2009225, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017262, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000137, HSYRA1000152, HSYRA1000178, HSYRA2000044, HSYRA2000137, HSYRA2000190, HSYRA2000221, HSYRA2000224, HSYRA2000237, HSYRA2000253, HSYRA2000255, HSYRA2000332, HSYRA2000371, HSYRA2000424, HSYRA2000507, HSYRA2000510, HSYRA2000605, HSYRA2000629, HSYRA2000640, HSYRA2000706, HSYRA2000743, HSYRA2000760, HSYRA2000787, HSYRA2000792, HSYRA2000832, HSYRA2000927, HSYRA2001103, HSYRA2001105, HSYRA2001142, HSYRA2001225, HSYRA2001255, HSYRA2001332, HSYRA2001353, HSYRA2001396, HSYRA2001420, HSYRA2001484, HSYRA2001552, HSYRA2001567, HSYRA2001574, HSYRA2001580, HSYRA2001615, HSYRA2001621, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000161, IMR321000165, IMR321000193, IMR321000207, IMR321000210, IMR321000230, IMR321000242, IMR321000253, IMR322000010, IMR322000072, IMR322000107, IMR322000121, IMR322000127, IMR322000144, IMR322000152, IMR322000164, IMR322000178, IMR322000223, IMR322000243, IMR322000302, IMR322000316, IMR322000374, IMR322000379, IMR322000383, IMR322000418, IMR322000518, IMR322000555, IMR322000604, IMR322000609, IMR322000656, IMR322000672, IMR322000742, IMR322000775, IMR322000811, IMR322000819, IMR322000838, IMR322000859, IMR322000863, IMR322000917, IMR322000918, IMR322000919, IMR322000935, IMR322000953, IMR322000984, IMR322001018, IMR322001049, IMR322001167, IMR322001185, IMR322001218, IMR322001245, IMR322001317, IMR322001318, IMR322001322, IMR322001332, IMR322001380, IMR322001438, IMR322001452, IMR322001491, IMR322001534, IMR322001541, IMR322001584, IMR322001665, IMR322001670, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322013053, IMR322013396, IMR322013731, IMR322013805, IMR322015523, IMR322016146, IMR322017049, IMR322018117, IMR322018192, IMR322019070, JCMLC2000273, KIDNE1000008, KIDNE1000012, KIDNE1000015, KIDNE1000028, KIDNE1000036, KIDNE1000064, KIDNE1000104, KIDNE1000121, KIDNE1000143, KIDNE1000145, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000252, KIDNE2000266, KIDNE2000287, KIDNE2000315, KIDNE2000317, KIDNE2000328, KIDNE2000330, KIDNE2000335, KIDNE2000349, KIDNE2000375, KIDNE2000383, KIDNE2000394, KIDNE2000403, KIDNE2000422, KIDNE2000493, KIDNE2000497, KIDNE2000510, KIDNE2000513, KIDNE2000517, KIDNE2000519, KIDNE2000555, KIDNE2000624, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2001117, KIDNE2001140, KIDNE2001162, KIDNE2001269, KIDNE2001361, KIDNE2001373, KIDNE2001636, KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002252, KIDNE2002262, KIDNE2002265, KIDNE2002438, KIDNE2002483, KIDNE2002725, KIDNE2002768, KIDNE2002795, KIDNE2002798, KIDNE2002845, KIDNE2002872, KIDNE2002882, KIDNE2002883, KIDNE2002980, KIDNE2002991, KIDNE2003185, KIDNE2003188, KIDNE2003233, KIDNE2003305, KIDNE2003316, KIDNE2003335, KIDNE2003357, KIDNE2003373, KIDNE2003377, KIDNE2003507, KIDNE2003752, KIDNE2003764, KIDNE2003837, KIDNE2003994, KIDNE2004034, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411, KIDNE2004475, KIDNE2004519, KIDNE2004520, KIDNE2004531, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006014, KIDNE2006039, KIDNE2006053, KIDNE2006062, KIDNE2006092, KIDNE2006145, KIDNE2006149, KIDNE2006210, KIDNE2006248, KIDNE2006299, KIDNE2006334, KIDNE2006353, KIDNE2006376, KIDNE2006378, KIDNE2006465, KIDNE2006580, KIDNE2006652, KIDNE2006687, KIDNE2006733, KIDNE2006760, KIDNE2006775, KIDNE2006811, KIDNE2006820, KIDNE2006880, KIDNE2007005, KIDNE2007040, KIDNE2007077, KIDNE2007186, KIDNE2007222, KIDNE2007328, KIDNE2007356, KIDNE2007422, KIDNE2007569, KIDNE2007810, KIDNE2007910, KIDNE2007944, KIDNE2007954, KIDNE2008022, KIDNE2008069, KIDNE2008072, KIDNE2008116, KIDNE2008117, KIDNE2008218, KIDNE2008315, KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009367, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014290, KIDNE2014298, KIDNE2014320, KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539, KIDNE2016570, KIDNE2016918, KIDNE2017007, KIDNE2017040, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018462, KIDNE2018493, KIDNE2018617, KIDNE2018678, KIDNE2018727, KIDNE2018863, KIDNE2018891, KIDNE2018989, KIDNE2018996, KIDNE2019074, KIDNE2019187, LIVER1000050, LIVER1000055, LIVER1000058, LIVER1000063, LIVER1000073, LIVER1000082, LIVER1000099, LIVER1000126, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER2000003, LIVER2000033, LIVER2000216, LIVER2000237, LIVER2000247, LIVER2000360, LIVER2000416, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2001835, LIVER2002638, LIVER2002644, LIVER2002660, LIVER2002702, LIVER2003008, LIVER2003065, LIVER2003234, LIVER2003511, LIVER2003524, LIVER2003568, LIVER2003581, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006251, LIVER2006410, LIVER2006469, LIVER2006644, LIVER2006764, LIVER2007415, LIVER2007548, LIVER2007568, LIVER2007721, LIVER2007783, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000096, MAMGL1000132, MAMGL1000151, MAMGL1000178, MAMGL1000182, MAMGL1000184, MESAN1000032, MESAN1000035, MESAN1000079, MESAN1000101, MESAN1000121, MESAN1000126, MESAN1000147, MESAN1000180, MESAN2000023, MESAN2000026, MESAN2000067, MESAN2000092, MESAN2000167, MESAN2000264, MESAN2000291, MESAN2000337, MESAN2000434, MESAN2000457, MESAN2000462, MESAN2000501, MESAN2000620, MESAN2000711, MESAN2000815, MESAN2000894, MESAN2000909, MESAN2001154, MESAN2001450, MESAN2001627, MESAN2001770, MESAN2001979, MESAN2001980, MESAN2002113, MESAN2002122, MESAN2002147, MESAN2002186, MESAN2002424, MESAN2002519, MESAN2002687, MESAN2002709, MESAN2002724, MESAN2002790, MESAN2002844, MESAN2002978, MESAN2003035, MESAN2003037, MESAN2003039, MESAN2003058, MESAN2003101, MESAN2003190, MESAN2003322, MESAN2003490, MESAN2003529, MESAN2003646, MESAN2003662, MESAN2003709, MESAN2003851, MESAN2004138, MESAN2004288, MESAN2004575, MESAN2005284, MESAN2005303, MESAN2005371, MESAN2005633, MESAN2005689, MESAN2005724, MESAN2005808, MESAN2005811, MESAN2005957, MESAN2005958, MESAN2006022, MESAN2006043, MESAN2006328, MESAN2006401, MESAN2006563, MESAN2006580, MESAN2006599, MESAN2006971, MESAN2007032, MESAN2007440, MESAN2007597, MESAN2007755, MESAN2008222, MESAN2008415, MESAN2008460, MESAN2008536, MESAN2008679, MESAN2008729, MESAN2008821, MESAN2008926, MESAN2008936, MESAN2009019, MESAN2009081, MESAN2009105, MESAN2009311, MESAN2009418, MESAN2009503, MESAN2009522, MESAN2010031, MESAN2010114, MESAN2010312, MESAN2010321, MESAN2010664, MESAN2010681, MESAN2011545, MESAN2011597, MESAN2011627, MESAN2011632, MESAN2011977, MESAN2011984, MESAN2012054, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016552, MESAN2016965, MESAN2017133, MESAN2017152, MESAN2017373, MESAN2017417, MESAN2018209, MESAN2018670, MESAN2018699, MESTC1000042, MESTC1000147, MESTC2000150, MESTC2000153, MESTC2000170, N1ESE2000698, NB9N41000011, NB9N41000047, NB9N41000121, NB9N41000135, NB9N41000142, NB9N41000146, NB9N42000104, NB9N42000122, NB9N42000196, NB9N42000281, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000102, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000004, NT2NE1000014, NT2NE1000018, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000195, NT2NE2000222, NT2NE2000284, NT2NE2000299, NT2NE2000327, NT2NE2000353, NT2NE2000356, NT2NE2000369, NT2NE2000374, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000586, NT2NE2000612, NT2NE2000658, NT2NE2000706, NT2NE2000763, NT2NE2000802, NT2NE2000808, NT2NE2000864, NT2NE2000909, NT2NE2000913, NT2NE2000950, NT2NE2000951, NT2NE2000980, NT2NE2001005, NT2NE2001021, NT2NE2001040, NT2NE2001049, NT2NE2001142, NT2NE2001156, NT2NE2001176, NT2NE2001180, NT2NE2001181, NT2NE2001188, NT2NE2001191, NT2NE2001247, NT2NE2001252, NT2NE2001324, NT2NE2001326, NT2NE2001337, NT2NE2001364, NT2NE2001372, NT2NE2001397, NT2NE2001403, NT2NE2001428, NT2NE2001432, NT2NE2001435, NT2NE2001442, NT2NE2001524, NT2NE2001530, NT2NE2001545, NT2NE2001598, NT2NE2001617, NT2NE2001623, NT2NE2001626, NT2NE2001634, NT2NE2001648, NT2NE2001655, NT2NE2001660, NT2NE2001666, NT2NE2001677, NT2NE2001697, NT2NE2001698, NT2NE2001723, NT2NE2001725, NT2NE2001793, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003185, NT2NE2003252, NT2NE2003280, NT2NE2003308, NT2NE2003309, NT2NE2003315, NT2NE2003393, NT2NE2003408, NT2NE2003457, NT2NE2003485, NT2NE2003540, NT2NE2003569, NT2NE2003571, NT2NE2003705, NT2NE2003752, NT2NE2003887, NT2NE2003921, NT2NE2004255, NT2NE2004378, NT2NE2004490, NT2NE2004519, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005419, NT2NE2005441, NT2NE2005517, NT2NE2005537, NT2NE2005555, NT2NE2005588, NT2NE2005676, NT2NE2005720, NT2NE2005784, NT2NE2005804, NT2NE2005821, NT2NE2005876, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006478, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010854, NT2NE2011033, NT2NE2011036, NT2NE2011107, NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018594, NT2NE2018739, NT2NE2018916, NT2NE2019056, NT2NE2019076, NT2NE2019091, NT2NE2019099, NT2RI1000016, NT2RI1000027, NT2RI1000127, NT2RI1000164, NT2RI1000172, NT2RI2000004, NT2RI2000007, NT2RI2000107, NT2RI2000116, NT2RI2000117, NT2RI2000128, NT2RI2000133, NT2RI2000167, NT2RI2000173, NT2RI2000187, NT2RI2000255, NT2RI2000263, NT2RI2000270, NT2RI2000276, NT2RI2000294, NT2RI2000313, NT2RI2000341, NT2RI2000344, NT2RI2000348, NT2RI2000412, NT2RI2000421, NT2RI2000480, NT2RI2000575, NT2RI2000588, NT2RI2000597, NT2RI2000669, NT2RI2000685, NT2RI2000688, NT2RI2000689, NT2RI2000704, NT2RI2000738, NT2RI2000775, NT2RI2000822, NT2RI2000829, NT2RI2000841, NT2RI2000865, NT2RI2000932, NT2RI2000987, NT2RI2001010, NT2RI2001017, NT2RI2001030, NT2RI2001083, NT2RI2001091, NT2RI2001167, NT2RI2001178, NT2RI2001230, NT2RI2001235, NT2RI2001244, NT2RI2001307, NT2RI2001409, NT2RI2001410, NT2RI2001422, NT2RI2001425, NT2RI2001449, NT2RI2001450, NT2RI2001474, NT2RI2001519, NT2RI2001595, NT2RI2001621, NT2RI2001624, NT2RI2001657, NT2RI2001726, NT2RI2001731, NT2RI2001769, NT2RI2001782, NT2RI2001836, NT2RI2001846, NT2RI2001859, NT2RI2001866, NT2RI2001910, NT2RI2001952, NT2RI2002022, NT2RI2002041, NT2RI2002120, NT2RI2002153, NT2RI2002179, NT2RI2002243, NT2RI2002252, NT2RI2002260, NT2RI2002266, NT2RI2002270, NT2RI2002334, NT2RI2002359, NT2RI2002391, NT2RI2002447, NT2RI2002481, NT2RI2002507, NT2RI2002517, NT2RI2002530, NT2RI2002540, NT2RI2002549, NT2RI2002554, NT2RI2002564, NT2RI2002585, NT2RI2002592, NT2RI2002602, NT2RI2002699, NT2RI2002729, NT2RI2002802, NT2RI2002819, NT2RI2002847, NT2RI2002852, NT2RI2002865, NT2RI2002923, NT2RI2002926, NT2RI2002970, NT2RI2003011, NT2RI2003019, NT2RI2003067, NT2RI2003184, NT2RI2003205, NT2RI2003222, NT2RI2003301, NT2RI2003317, NT2RI2003344, NT2RI2003360, NT2RI2003361, NT2RI2003383, NT2RI2003392, NT2RI2003399, NT2RI2003419, NT2RI2003481, NT2RI2003487, NT2RI2003655, NT2RI2003667, NT2RI2003695, NT2RI2003884, NT2RI2003921, NT2RI2003973, NT2RI2004059, NT2RI2004078, NT2RI2004079, NT2RI2004093, NT2RI2004136, NT2RI2004157, NT2RI2004188, NT2RI2004190, NT2RI2004209, NT2RI2004284, NT2RI2004290, NT2RI2004304, NT2RI2004312, NT2RI2004381, NT2RI2004398, NT2RI2004410, NT2RI2004442, NT2RI2004468, NT2RI2004606, NT2RI2004608, NT2RI2004618, NT2RI2004783, NT2RI2004818, NT2RI2004840, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005116, NT2RI2005130, NT2RI2005150, NT2RI2005166, NT2RI2005239, NT2RI2005246, NT2RI2005315, NT2RI2005358, NT2RI2005368, NT2RI2005382, NT2RI2005473, NT2RI2005520, NT2RI2005564, NT2RI2005579, NT2RI2005621, NT2RI2005628, NT2RI2005647, NT2RI2005670, NT2RI2005713, NT2RI2005772, NT2RI2005811, NT2RI2005816, NT2RI2005851, NT2RI2005966, NT2RI2006070, NT2RI2006071, NT2RI2006081, NT2RI2006126, NT2RI2006127, NT2RI2006183, NT2RI2006257, NT2RI2006271, NT2RI2006294, NT2RI2006345, NT2RI2006403, NT2RI2006412, NT2RI2006445, NT2RI2006487, NT2RI2006506, NT2RI2006553, NT2RI2006565, NT2RI2006640, NT2RI2006647, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006825, NT2RI2006838, NT2RI2006853, NT2RI2006855, NT2RI2006856, NT2RI2006943, NT2RI2006973, NT2RI2007046, NT2RI2007048, NT2RI2007096, NT2RI2007116, NT2RI2007133, NT2RI2007148, NT2RI2007214, NT2RI2007277, NT2RI2007303, NT2RI2007334, NT2RI2007377, NT2RI2007386, NT2RI2007406, NT2RI2007439, NT2RI2007468, NT2RI2007469, NT2RI2007498, NT2RI2007507, NT2RI2007589, NT2RI2007723, NT2RI2007729, NT2RI2007751, NT2RI2007754, NT2RI2007827, NT2RI2007853, NT2RI2007877, NT2RI2007884, NT2RI2007891, NT2RI2007956, NT2RI2007965, NT2RI2007987, NT2RI2008007, NT2RI2008050, NT2RI2008171, NT2RI2008188, NT2RI2008204, NT2RI2008221, NT2RI2008336, NT2RI2008455, NT2RI2008481, NT2RI2008502, NT2RI2008566, NT2RI2008622, NT2RI2008714, NT2RI2008801, NT2RI2008808, NT2RI2008812, NT2RI2008841, NT2RI2008942, NT2RI2008952, NT2RI2009005, NT2RI2009037, NT2RI2009065, NT2RI2009066, NT2RI2009083, NT2RI2009101, NT2RI2009103, NT2RI2009144, NT2RI2009151, NT2RI2009173, NT2RI2009215, NT2RI2009239, NT2RI2009259, NT2RI2009281, NT2RI2009289, NT2RI2009301, NT2RI2009402, NT2RI2009406, NT2RI2009517, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012659, NT2RI2012726, NT2RI2012990, NT2RI2013345, NT2RI2013357, NT2RI2013373, NT2RI2014247, NT2RI2014551, NT2RI2014683, NT2RI2014733, NT2RI2015239, NT2RI2015342, NT2RI2015533, NT2RI2016128, NT2RI2016134, NT2RI2017529, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006284, NT2RI3006340, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000005, NT2RP6000008, NT2RP6000017, NT2RP6000032, NT2RP6000033, NT2RP6000039, NT2RP6000043, NT2RP6000059, NT2RP6000060, NT2RP6000072, NT2RP6000077, NT2RP6000078, NT2RP6000085, NT2RP6000086, NT2RP6000100, NT2RP6000109, NT2RP6000110, NT2RP6000119, NT2RP6000127, NT2RP6000130, NT2RP6000140, NT2RP6000150, NT2RP7000041, NT2RP7000069, NT2RP7000076, NT2RP7000086, NT2RP7000109, NT2RP7000112, NT2RP7000173, NT2RP7000238, NT2RP7000259, NT2RP7000271, NT2RP7000311, NT2RP7000359, NT2RP7000364, NT2RP7000380, NT2RP7000391, NT2RP7000425, NT2RP7000477, NT2RP7000579, NT2RP7000586, NT2RP7000600, NT2RP7000624, NT2RP7000812, NT2RP7000926, NT2RP7001074, NT2RP7001080, NT2RP7001156, NT2RP7001166, NT2RP7001231, NT2RP7001283, NT2RP7001306, NT2RP7001319, NT2RP7001335, NT2RP7001364, NT2RP7001475, NT2RP7001532, NT2RP7001591, NT2RP7001602, NT2RP7001678, NT2RP7001856, NT2RP7001962, NT2RP7002064, NT2RP7002151, NT2RP7002163, NT2RP7002243, NT2RP7002282, NT2RP7002376, NT2RP7002379, NT2RP7002449, NT2RP7002450, NT2RP7002478, NT2RP7002554, NT2RP7002594, NT2RP7002603, NT2RP7002619, NT2RP7002650, NT2RP7002738, NT2RP7002779, NT2RP7002802, NT2RP7002829, NT2RP7002841, NT2RP7002875, NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003203, NT2RP7003261, NT2RP7003439, NT2RP7003511, NT2RP7003629, NT2RP7003647, NT2RP7003680, NT2RP7003688, NT2RP7003724, NT2RP7004027, NT2RP7004080, NT2RP7004114, NT2RP7004196, NT2RP7004233, NT2RP7004260, NT2RP7004348, NT2RP7004352, NT2RP7004358, NT2RP7004396, NT2RP7004481, NT2RP7004541, NT2RP7004559, NT2RP7004641, NT2RP7004656, NT2RP7004687, NT2RP7004722, NT2RP7004766, NT2RP7004884, NT2RP7004911, NT2RP7004915, NT2RP7004925, NT2RP7004961, NT2RP7004964, NT2RP7004975, NT2RP7005118, NT2RP7005205, NT2RP7005219, NT2RP7005323, NT2RP7005468, NT2RP7005493, NT2RP7005513, NT2RP7005529, NT2RP7005629, NT2RP7005631, NT2RP7005669, NT2RP7005675, NT2RP7005750, NT2RP7005846, NT2RP7006033, NT2RP7006160, NT2RP7006162, NT2RP7006188, NT2RP7006223, NT2RP7006296, NT2RP7006304, NT2RP7006374, NT2RP7006395, NT2RP7006408, NT2RP7006457, NT2RP7006490, NT2RP7006539, NT2RP7006547, NT2RP7006601, NT2RP7006621, NT2RP7006636, NT2RP7006701, NT2RP7006853, NT2RP7006886, NT2RP7006887, NT2RP7006980, NT2RP7006995, NT2RP7007114, NT2RP7007221, NT2RP7007226, NT2RP7007252, NT2RP7007269, NT2RP7007310, NT2RP7007359, NT2RP7007381, NT2RP7007406, NT2RP7007480, NT2RP7007504, NT2RP7007530, NT2RP7007537, NT2RP7007580, NT2RP7007594, NT2RP7007610, NT2RP7007617, NT2RP7007643, NT2RP7007766, NT2RP7007842, NT2RP7007925, NT2RP7007930, NT2RP7007975, NT2RP7008013, NT2RP7008015, NT2RP7008133, NT2RP7008137, NT2RP7008142, NT2RP7008144, NT2RP7008161, NT2RP7008167, NT2RP7008315, NT2RP7008360, NT2RP7008396, NT2RP7008406, NT2RP7008435, NT2RP7008441, NT2RP7008454, NT2RP7008487, NT2RP7008543, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009087, NT2RP7009108, NT2RP7009147, NT2RP7009149, NT2RP7009168, NT2RP7009215, NT2RP7009225, NT2RP7009236, NT2RP7009253, NT2RP7009267, NT2RP7009322, NT2RP7009363, NT2RP7009370, NT2RP7009373, NT2RP7009394, NT2RP7009397, NT2RP7009429, NT2RP7009439, NT2RP7009466, NT2RP7009482, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015503, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000033, NTONG1000052, NTONG1000075, NTONG1000087, NTONG1000123, NTONG1000130, NTONG1000161, NTONG1000172, NTONG1000182, NTONG1000213, NTONG1000214, NTONG1000246, NTONG1000247, NTONG1000257, NTONG1000264, NTONG2000040, NTONG2000107, NTONG2000231, NTONG2000265, NTONG2000334, NTONG2000363, NTONG2000413, NTONG2000492, NTONG2000531, NTONG2000583, NTONG2000800, NTONG2000858, NTONG2000878, NTONG2000966, NTONG2000985, NTONG2001014, NTONG2001079, NTONG2001137, NTONG2001157, NTONG2001173, NTONG2001220, NTONG2001222, NTONG2001362, NTONG2001428, NTONG2001532, NTONG2001587, NTONG2001612, NTONG2001619, NTONG2001642, NTONG2001657, NTONG2001676, NTONG2001762, NTONG2002278, NTONG2002582, NTONG2002807, NTONG2002948, NTONG2002970, NTONG2002985, NTONG2003158, NTONG2003210, NTONG2003316, NTONG2003409, NTONG2003454, NTONG2003515, NTONG2003805, NTONG2003852, NTONG2003897, NTONG2003928, NTONG2004095, NTONG2004308, NTONG2004521, NTONG2004690, NTONG2004806, NTONG2004829, NTONG2004844, NTONG2004918, NTONG2004991, NTONG2005062, NTONG2005086, NTONG2005137, NTONG2005153, NTONG2005265, NTONG2005277, NTONG2005363, NTONG2005373, NTONG2005391, NTONG2005480, NTONG2005497, NTONG2005520, NTONG2005577, NTONG2005657, NTONG2005801, NTONG2005822, NTONG2005897, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008522, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030, OCBBF1000042, OCBBF1000049, OCBBF1000054, OCBBF1000067, OCBBF1000080, OCBBF1000085, OCBBF1000086, OCBBF1000091, OCBBF1000104, OCBBF1000118, OCBBF1000119, OCBBF1000122, OCBBF1000130, OCBBF1000138, OCBBF1000145, OCBBF1000146, OCBBF1000153, OCBBF1000175, OCBBF1000185, OCBBF1000188, OCBBF1000192, OCBBF1000254, OCBBF1000315, OCBBF2000015, OCBBF2000126, OCBBF2000178, OCBBF2000231, OCBBF2000277, OCBBF2000287, OCBBF2000323, OCBBF2000452, OCBBF2000467, OCBBF2000677, OCBBF2000703, OCBBF2000712, OCBBF2000719, OCBBF2000824, OCBBF2000831, OCBBF2000904, OCBBF2000982, OCBBF2000986, OCBBF2000998, OCBBF2001075, OCBBF2001101, OCBBF2001124, OCBBF2001140, OCBBF2001166, OCBBF2001176, OCBBF2001186, OCBBF2001196, OCBBF2001210, OCBBF2001252, OCBBF2001307, OCBBF2001323, OCBBF2001389, OCBBF2001402, OCBBF2001408, OCBBF2001416, OCBBF2001473, OCBBF2001494, OCBBF2001527, OCBBF2001528, OCBBF2001586, OCBBF2001639, OCBBF2001681, OCBBF2001687, OCBBF2001706, OCBBF2001749, OCBBF2001794, OCBBF2001910, OCBBF2001931, OCBBF2001961, OCBBF2001983, OCBBF2002015, OCBBF2002083, OCBBF2002086, OCBBF2002124, OCBBF2002161, OCBBF2002290, OCBBF2002357, OCBBF2002376, OCBBF2002615, OCBBF2002626, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003518, OCBBF2003543, OCBBF2003593, OCBBF2003744, OCBBF2003819, OCBBF2003925, OCBBF2004038, OCBBF2004104, OCBBF2004147, OCBBF2004168, OCBBF2004235, OCBBF2004259, OCBBF2004273, OCBBF2004385, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005546, OCBBF2005843, OCBBF2005901, OCBBF2005956, OCBBF2006005, OCBBF2006030, OCBBF2006058, OCBBF2006113, OCBBF2006148, OCBBF2006151, OCBBF2006154, OCBBF2006172, OCBBF2006214, OCBBF2006241, OCBBF2006313, OCBBF2006388, OCBBF2006438, OCBBF2006567, OCBBF2006624, OCBBF2006639, OCBBF2006660, OCBBF2006764, OCBBF2006849, OCBBF2006859, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007414, OCBBF2007415, OCBBF2007428, OCBBF2007478, OCBBF2007485, OCBBF2007520, OCBBF2007555, OCBBF2007610, OCBBF2007622, OCBBF2007756, OCBBF2007762, OCBBF2007931, OCBBF2007946, OCBBF2008005, OCBBF2008041, OCBBF2008116, OCBBF2008138, OCBBF2008144, OCBBF2008283, OCBBF2008330, OCBBF2008340, OCBBF2008366, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770, OCBBF2008775, OCBBF2008790, OCBBF2008814, OCBBF2008822, OCBBF2009095, OCBBF2009115, OCBBF2009242, OCBBF2009352, OCBBF2009391, OCBBF2009424, OCBBF2009536, OCBBF2009571, OCBBF2009583, OCBBF2009603, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010830, OCBBF2010841, OCBBF2010843, OCBBF2010858, OCBBF2010863, OCBBF2010871, OCBBF2010931, OCBBF2010945, OCBBF2010978, OCBBF2010989, OCBBF2011021, OCBBF2011073, OCBBF2011137, OCBBF2011160, OCBBF2011177, OCBBF2011228, OCBBF2011232, OCBBF2011283, OCBBF2011311, OCBBF2011536, OCBBF2011625, OCBBF2011669, OCBBF2011685, OCBBF2011706, OCBBF2011722, OCBBF2011759, OCBBF2011767, OCBBF2011855, OCBBF2011872, OCBBF2011887, OCBBF2011897, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012553, OCBBF2012678, OCBBF2012704, OCBBF2012714, OCBBF2012755, OCBBF2012812, OCBBF2012821, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789, OCBBF2013843, OCBBF2013883, OCBBF2013926, OCBBF2014052, OCBBF2014089, OCBBF2014322, OCBBF2014386, OCBBF2014541, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016841, OCBBF2016928, OCBBF2017035, OCBBF2017055, OCBBF2017069, OCBBF2017306, OCBBF2017325, OCBBF2017398, OCBBF2017458, OCBBF2017489, OCBBF2017516, OCBBF2017665, OCBBF2017754, OCBBF2017791, OCBBF2017815, OCBBF2017882, OCBBF2017888, OCBBF2017893, OCBBF2017920, OCBBF2018012, OCBBF2018014, OCBBF2018084, OCBBF2018167, OCBBF2018206, OCBBF2018229, OCBBF2018234, OCBBF2018362, OCBBF2018380, OCBBF2018563, OCBBF2018581, OCBBF2018618, OCBBF2018663, OCBBF2018687, OCBBF2018827, OCBBF2018828, OCBBF2018873, OCBBF2018934, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF3000097, OCBBF3000213, OCBBF3000296, OCBBF3000323, OCBBF3000372, OCBBF3000380, OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004487, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000174, PEBLM1000180, PEBLM2000030, PEBLM2000112, PEBLM2000126, PEBLM2000170, PEBLM2000180, PEBLM2000213, PEBLM2000222, PEBLM2000248, PEBLM2000267, PEBLM2000270, PEBLM2000321, PEBLM2000326, PEBLM2000333, PEBLM2000338, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004452, PEBLM2004497, PEBLM2004666, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006366, PEBLM2006709, PEBLM2006912, PEBLM2007112, PEBLM2007140, PEBLM2007188, PEBLM2007296, PEBLM2007437, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005-370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000066, PLACE5000067, PLACE5000068, PLACE5000070, PLACE5000080, PLACE5000105, PLACE5000113, PLACE5000115, PLACE5000116, PLACE5000129, PLACE5000139, PLACE5000153, PLACE5000159, PLACE5000170, PLACE5000171, PLACE5000172, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000296, PLACE6000314, PLACE6000325, PLACE6000371, PLACE6000414, PLACE6000424, PLACE6000426, PLACE6000429, PLACE6000450, PLACE6000463, PLACE6000523, PLACE6000550, PLACE6000552, PLACE6000555, PLACE6000619, PLACE6000630, PLACE6000953, PLACE6001118, PLACE6001153, PLACE6001185, PLACE6001262, PLACE6001281, PLACE6001294, PLACE6001443, PLACE6001536, PLACE6001557, PLACE6001712, PLACE6001740, PLACE6001823, PLACE6001886, PLACE6001925, PLACE6001933, PLACE6002011, PLACE6002016, PLACE6002056, PLACE6002102, PLACE6002157, PLACE6002312, PLACE6002323, PLACE6002345, PLACE6002419, PLACE6002462, PLACE6002647, PLACE6002692, PLACE6002699, PLACE6002710, PLACE6002871, PLACE6002949, PLACE6002960, PLACE6003004, PLACE6003038, PLACE6003077, PLACE6003094, PLACE6003109, PLACE6003204, PLACE6003218, PLACE6003372, PLACE6003383, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004101, PLACE6004219, PLACE6004312, PLACE6004336, PLACE6004368, PLACE6004380, PLACE6004396, PLACE6004397, PLACE6004454, PLACE6004464, PLACE6004491, PLACE6004663, PLACE6004687, PLACE6004738, PLACE6004931, PLACE6004966, PLACE6005007, PLACE6005029, PLACE6005108, PLACE6005231, PLACE6005234, PLACE6005283, PLACE6005294, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006077, PLACE6006137, PLACE6006158, PLACE6006186, PLACE6006217, PLACE6006222, PLACE6006266, PLACE6006287, PLACE6006394, PLACE6006418, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007436, PLACE6007482, PLACE6007687, PLACE6007787, PLACE6007798, PLACE6007807, PLACE6008036, PLACE6008094, PLACE6008126, PLACE6008285, PLACE6008444, PLACE6008453, PLACE6008640, PLACE6008750, PLACE6008768, PLACE6008775, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010484, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011057, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016254, PLACE6016383, PLACE6016942, PLACE6016984, PLACE6017002, PLACE6017431, PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018497, PLACE6018769, PLACE6018834, PLACE6018843, PLACE6018863, PLACE6018938, PLACE6019195, PLACE6019385, PLACE6019542, PLACE6019600, PLACE6019674, PLACE6019676, PLACE6019701, PLACE6019932, PLACE6020031, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000097, PROST1000110, PROST1000123, PROST1000136, PROST1000144, PROST1000145, PROST1000152, PROST1000167, PROST1000184, PROST1000199, PROST1000215, PROST1000217, PROST1000220, PROST1000226, PROST1000246, PROST1000262, PROST1000272, PROST1000298, PROST1000322, PROST1000334, PROST1000343, PROST1000362, PROST1000377, PROST1000383, PROST1000412, PROST1000419, PROST1000451, PROST1000480, PROST1000485, PROST1000493, PROST1000512, PROST1000527, PROST1000528, PROST1000536, PROST1000559, PROST1000575, PROST2000036, PROST2000053, PROST2000067, PROST2000079, PROST2000105, PROST2000143, PROST2000162, PROST2000165, PROST2000203, PROST2000206, PROST2000212, PROST2000241, PROST2000267, PROST2000273, PROST2000274, PROST2000277, PROST2000284, PROST2000325, PROST2000337, PROST2000432, PROST2000452, PROST2000463, PROST2000470, PROST2000505, PROST2000567, PROST2000572, PROST2000717, PROST2000761, PROST2000835, PROST2000893, PROST2000961, PROST2001002, PROST2001116, PROST2001138, PROST2001180, PROST2001213, PROST2001283, PROST2001414, PROST2001415, PROST2001465, PROST2001521, PROST2001540, PROST2001552, PROST2001599, PROST2001611, PROST2001636, PROST2001796, PROST2001805, PROST2001823, PROST2001883, PROST2001899, PROST2001997, PROST2001998, PROST2002078, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002960, PROST2003102, PROST2003117, PROST2003210, PROST2003302, PROST2003303, PROST2003324, PROST2003338, PROST2003340, PROST2003396, PROST2003428, PROST2003472, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004258, PROST2004270, PROST2004332, PROST2004416, PROST2004481, PROST2004526, PROST2004570, PROST2004727, PROST2004739, PROST2004742, PROST2004744, PROST2004817, PROST2005039, PROST2005067, PROST2005076, PROST2005093, PROST2005121, PROST2005131, PROST2005143, PROST2005228, PROST2005272, PROST2005285, PROST2005426, PROST2005466, PROST2005604, PROST2005630, PROST2005683, PROST2005788, PROST2005793, PROST2005880, PROST2005886, PROST2005904, PROST2005919, PROST2005943, PROST2006008, PROST2006020, PROST2006030, PROST2006060, PROST2006196, PROST2006201, PROST2006260, PROST2006282, PROST2006343, PROST2006450, PROST2006491, PROST2006510, PROST2006579, PROST2006688, PROST2006766, PROST2006782, PROST2006933, PROST2006988, PROST2007122, PROST2007200, PROST2007237, PROST2007248, PROST2007289, PROST2007317, PROST2007328, PROST2007382, PROST2007389, PROST2007416, PROST2007528, PROST2007818, PROST2007871, PROST2007950, PROST2007956, PROST2007974, PROST2007988, PROST2008079, PROST2008088, PROST2008243, PROST2008245, PROST2008268, PROST2008271, PROST2008360, PROST2008447, PROST2008468, PROST2008472, PROST2008516, PROST2008724, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010579, PROST2010606, PROST2010782, PROST2010885, PROST2011012, PROST2011038, PROST2011105, PROST2011297, PROST2011410, PROST2011439, PROST2011482, PROST2011577, PROST2011631, PROST2011660, PROST2012005, PROST2012016, PROST2012088, PROST2012140, PROST2012157, PROST2012190, PROST2012249, PROST2012353, PROST2012400, PROST2012448, PROST2012504, PROST2012542, PROST2012550, PROST2012740, PROST2012745, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015137, PROST2015198, PROST2015243, PROST2015246, PROST2015251, PROST2015287, PROST2015332, PROST2015457, PROST2015464, PROST2015537, PROST2015545, PROST2015551, PROST2015636, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2016980, PROST2017098, PROST2017128, PROST2017367, PROST2017413, PROST2017441, PROST2017529, PROST2017578, PROST2017612, PROST2017617, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019164, PROST2019253, PROST2019296, PROST2019398, PROST2019487, PROST2019781, PUAEN1000039, PUAEN1000081, PUAEN1000085, PUAEN1000087, PUAEN1000121, PUAEN1000147, PUAEN1000164, PUAEN1000175, PUAEN1000239, PUAEN1000275, PUAEN1000276, PUAEN1000288, PUAEN2000080, PUAEN2000152, PUAEN2000175, PUAEN2000247, PUAEN2000312, PUAEN2000394, PUAEN2000594, PUAEN2000684, PUAEN2000942, PUAEN2001260, PUAEN2001345, PUAEN2001526, PUAEN2001586, PUAEN2002473, PUAEN2002489, PUAEN2002552, PUAEN2002568, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007350, PUAEN2007785, PUAEN2007898, PUAEN2008123, PUAEN2008228, PUAEN2008314, PUAEN2008515, PUAEN2008815, PUAEN2008939, PUAEN2008980, PUAEN2008985, PUAEN2009174, PUAEN2009348, PUAEN2009534, PUAEN2009655, PUAEN2009795, PUAEN2009852, PUAEN2010346, PUAEN2010824, RECTM1000051, RECTM1000141, RECTM2000220, RECTM2000349, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000047, SALGL1000065, SALGL1000107, SALGL1000171, SKMUS1000014, SKMUS1000022, SKMUS1000030, SKMUS1000064, SKMUS1000067, SKMUS1000084, SKMUS1000102, SKMUS1000104, SKMUS1000110, SKMUS1000118, SKMUS1000124, SKMUS1000129, SKMUS1000176, SKMUS1000177, SKMUS1000186, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000361, SKMUS2000365, SKMUS2000380, SKMUS2000390, SKMUS2000416, SKMUS2000458, SKMUS2000467, SKMUS2000468, SKMUS2000484, SKMUS2000515, SKMUS2000679, SKMUS2000690, SKMUS2000701, SKMUS2000705, SKMUS2000710, SKMUS2000724, SKMUS2000726, SKMUS2000757, SKMUS2000774, SKMUS2000821, SKMUS2000847, SKMUS2000863, SKMUS2000873, SKMUS2000894, SKMUS2000898, SKMUS2000902, SKMUS2000945, SKMUS2000954, SKMUS2000961, SKMUS2000973, SKMUS2000980, SKMUS2001008, SKMUS2001014, SKMUS2001129, SKMUS2001147, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001501, SKMUS2001528, SKMUS2001543, SKMUS2001580, SKMUS2001585, SKMUS2001608, SKMUS2001631, SKMUS2001634, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000110, SKNMC1000123, SKNMC1000124, SKNMC1000159, SKNMC1000168, SKNMC1000229, SKNMC1000251, SKNMC1000264, SKNMC2000065, SKNMC2000224, SKNMC2000256, SKNMC2000305, SKNMC2000322, SKNMC2000356, SKNMC2000374, SKNMC2000583, SKNMC2000593, SKNMC2000612, SKNMC2000622, SKNMC2000966, SKNMC2001113, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000002, SKNSH1000086, SKNSH1000174, SKNSH1000301, SKNSH1000308, SKNSH1000416, SKNSH2000051, SKNSH2000101, SKNSH2000151, SKNSH2000163, SKNSH2000250, SKNSH2000347, SKNSH2000482, SKNSH2000516, SKNSH2000550, SKNSH2000716, SKNSH2000819, SKNSH2000971, SKNSH2001222, SKNSH2001931, SKNSH2002054, SKNSH2002866, SKNSH2003064, SKNSH2003174, SKNSH2003422, SKNSH2003466, SKNSH2003483, SKNSH2003528, SKNSH2003633, SKNSH2004039, SKNSH2005061, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008043, SKNSH2008969, SKNSH2009197, SKNSH2009357, SKNSH2009435, SKNSH2009991, SKNSH2010015, SMINT1000012, SMINT1000016, SMINT1000039, SMINT1000042, SMINT1000048, SMINT1000051, SMINT1000054, SMINT1000057, SMINT1000071, SMINT1000075, SMINT1000078, SMINT1000091, SMINT1000100, SMINT1000103, SMINT1000118, SMINT1000131, SMINT1000137, SMINT1000192, SMINT2000007, SMINT2000018, SMINT2000032, SMINT2000040, SMINT2000060, SMINT2000073, SMINT2000084, SMINT2000145, SMINT2000159, SMINT2000176, SMINT2000185, SMINT2000227, SMINT2000229, SMINT2000232, SMINT2000239, SMINT2000267, SMINT2000277, SMINT2000396, SMINT2000400, SMINT2000454, SMINT2000518, SMINT2000529, SMINT2000530, SMINT2000537, SMINT2000538, SMINT2000541, SMINT2000545, SMINT2000558, SMINT2000562, SMINT2000568, SMINT2000609, SMINT2000629, SMINT2000694, SMINT2000747, SMINT2000811, SMINT2000824, SMINT2000933, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001818, SMINT2001950, SMINT2002126, SMINT2002159, SMINT2002202, SMINT2002210, SMINT2002224, SMINT2002281, SMINT2002311, SMINT2002314, SMINT2002328, SMINT2002414, SMINT2002457, SMINT2002620, SMINT2002689, SMINT2002743, SMINT2002778, SMINT2002880, SMINT2002882, SMINT2002884, SMINT2002899, SMINT2002974, SMINT2003003, SMINT2003128, SMINT2003150, SMINT2003317, SMINT2003386, SMINT2003423, SMINT2003505, SMINT2003551, SMINT2003569, SMINT2003641, SMINT2003644, SMINT2003866, SMINT2003905, SMINT2004037, SMINT2004047, SMINT2004086, SMINT2004280, SMINT2004299, SMINT2004339, SMINT2004350, SMINT2004414, SMINT2004473, SMINT2004547, SMINT2004583, SMINT2004589, SMINT2004729, SMINT2004781, SMINT2004891, SMINT2004909, SMINT2004972, SMINT2004992, SMINT2005075, SMINT2005161, SMINT2005174, SMINT2005213, SMINT2005330, SMINT2005387, SMINT2005402, SMINT2005405, SMINT2005621, SMINT2005623, SMINT2005624, SMINT2005800, SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2008921, SMINT2009119, SMINT2009121, SMINT2009212, SMINT2009216, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010901, SMINT2010959, SMINT2010997, SMINT2011033, SMINT2011066, SMINT2011273, SMINT2011311, SMINT2011406, SMINT2011509, SMINT2011588, SMINT2011656, SMINT2011888, SMINT2011958, SMINT2012040, SMINT2012122, SMINT2012179, SMINT2012195, SMINT2012285, SMINT2012291, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000134, SPLEN1000139, SPLEN1000143, SPLEN1000144, SPLEN1000145, SPLEN1000156, SPLEN1000163, SPLEN1000165, SPLEN1000166, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000222, SPLEN2000242, SPLEN2000243, SPLEN2000247, SPLEN2000258, SPLEN2000267, SPLEN2000270, SPLEN2000307, SPLEN2000341, SPLEN2000350, SPLEN2000357, SPLEN2000364, SPLEN2000402, SPLEN2000420, SPLEN2000443, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695, SPLEN2000698, SPLEN2000839, SPLEN2000882, SPLEN2001135, SPLEN2001141, SPLEN2001157, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001689, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002335, SPLEN2002343, SPLEN2002354, SPLEN2002385, SPLEN2002419, SPLEN2002451, SPLEN2002462, SPLEN2002463, SPLEN2002467, SPLEN2002493, SPLEN2002662, SPLEN2002695, SPLEN2002707, SPLEN2002744, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004220, SPLEN2004343, SPLEN2004346, SPLEN2004368, SPLEN2004611, SPLEN2004662, SPLEN2004773, SPLEN2004844, SPLEN2004880, SPLEN2004984, SPLEN2005009, SPLEN2005227, SPLEN2005272, SPLEN2005342, SPLEN2005416, SPLEN2005429, SPLEN2005450, SPLEN2005474, SPLEN2005560, SPLEN2005727, SPLEN2005783, SPLEN2005790, SPLEN2005806, SPLEN2005818, SPLEN2005838, SPLEN2005869, SPLEN2005927, SPLEN2005939, SPLEN2006143, SPLEN2006232, SPLEN2006268, SPLEN2006283, SPLEN2006305, SPLEN2006325, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007498, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008591, SPLEN2008786, SPLEN2009081, SPLEN2009088, SPLEN2009163, SPLEN2009315, SPLEN2009340, SPLEN2009513, SPLEN2009541, SPLEN2009548, SPLEN2009555, SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011419, SPLEN2011422, SPLEN2011672, SPLEN2011678, SPLEN2011737, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012523, SPLEN2012524, SPLEN2012525, SPLEN2012571, SPLEN2012606, SPLEN2012611, SPLEN2012619, SPLEN2012624, SPLEN2012725, SPLEN2012743, SPLEN2012756, SPLEN2012800, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014080, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014911, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016268, SPLEN2016304, SPLEN2016356, SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016773, SPLEN2016781, SPLEN2016840, SPLEN2016863, SPLEN2016922, SPLEN2016932, SPLEN2016972, SPLEN2016973, SPLEN2017043, SPLEN2017104, SPLEN2017121, SPLEN2017147, SPLEN2017187, SPLEN2017189, SPLEN2017212, SPLEN2017318, SPLEN2017351, SPLEN2017426, SPLEN2017452, SPLEN2017538, SPLEN2017592, SPLEN2017613, SPLEN2017643, SPLEN2017687, SPLEN2017740, SPLEN2017781, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019169, SPLEN2019257, SPLEN2019311, SPLEN2019323, SPLEN2019349, SPLEN2019375, SPLEN2019379, SPLEN2019471, SPLEN2019480, SPLEN2019505, SPLEN2019571, SPLEN2019575, SPLEN2019709, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021194, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027113, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028466, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031674, SPLEN2031724, SPLEN2031780, SPLEN2032036, SPLEN2032112, SPLEN2032154, SPLEN2032321, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042598, SPLEN2042714, SPLEN2042920, STOMA1000013, STOMA1000052, STOMA1000074, STOMA1000091, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000121, STOMA2000148, STOMA2000168, STOMA2000183, STOMA2000188, STOMA2000257, STOMA2000279, STOMA2000289, STOMA2000383, STOMA2000386, STOMA2000395, STOMA2000396, STOMA2000478, STOMA2000482, STOMA2000529, STOMA2000539, STOMA2000567, STOMA2000640, STOMA2000678, STOMA2000686, STOMA2000888, STOMA2001025, STOMA2001389, STOMA2002052, STOMA2002141, STOMA2002166, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049, SYNOV1000118, SYNOV1000124, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001033, SYNOV2001035, SYNOV2001088, SYNOV2001111, SYNOV2001212, SYNOV2001251, SYNOV2001262, SYNOV2001300, SYNOV2001356, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001153, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007671, SYNOV4007711, SYNOV4008245, SYNOV4008336, SYNOV4009129, SYNOV4009139, SYNOV4009298, SYNOV4009575, T1ESE2000116, T1ESE2000609, T1ESE2000904, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002273, TESOP2002451, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000023, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000109, TESTI1000125, TESTI1000127, TESTI1000128, TESTI1000131, TESTI1000138, TESTI1000142, TESTI1000157, TESTI1000163, TESTI1000168, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000172, TESTI2000177, TESTI2000179, TESTI2000184, TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082, TESTI2001120, TESTI2001134, TESTI2001141, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001862, TESTI2001869, TESTI2001879, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002365, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002707, TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003061, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003413, TESTI2003418, TESTI2003419, TESTI2003432, TESTI2003454, TESTI2003475, TESTI2003476, TESTI2003498, TESTI2003512, TESTI2003533, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004080, TESTI2004085, TESTI2004089, TESTI2004111, TESTI2004119, TESTI2004122, TESTI2004153, TESTI2004163, TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994, TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005408, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005743, TESTI2005759, TESTI2005767, TESTI2005775, TESTI2005784, TESTI2005788, TESTI2005827, TESTI2005835, TESTI2005860, TESTI2005892, TESTI2005908, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006453, TESTI2006457, TESTI2006460, TESTI2006465, TESTI2006483, TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006748, TESTI2006774, TESTI2006853, TESTI2006866, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007320, TESTI2007346, TESTI2007358, TESTI2007402, TESTI2007405, TESTI2007407, TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009474, TESTI2009477, TESTI2009497, TESTI2009502, TESTI2009511, TESTI2009520, TESTI2009544, TESTI2009545, TESTI2009551, TESTI2009585, TESTI2009588, TESTI2009683, TESTI2009727, TESTI2009739, TESTI2009785, TESTI2009812, TESTI2009835, TESTI2009853, TESTI2009935, TESTI2009977, TESTI2010009, TESTI2010154, TESTI2010180, TESTI2010239, TESTI2010280, TESTI2010354, TESTI2010396, TESTI2010400, TESTI2010409, TESTI2010513, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012155, TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257, TESTI2013264, TESTI2013268, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988, TESTI2015042, TESTI2015092, TESTI2015105, TESTI2015180, TESTI2015246, TESTI2015249, TESTI2015327, TESTI2015331, TESTI2015335, TESTI2015350, TESTI2015375, TESTI2015437, TESTI2015590, TESTI2015610, TESTI2015626, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018014, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018462, TESTI2018475, TESTI2018482, TESTI2018521, TESTI2018565, TESTI2018581, TESTI2018611, TESTI2018687, TESTI2018697, TESTI2018838, TESTI2018867, TESTI2018941, TESTI2018957, TESTI2018974, TESTI2018975, TESTI2018997, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021124, TESTI2021138, TESTI2021297, TESTI2021315, TESTI2021358, TESTI2021425, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025448, TESTI2025454, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026534, TESTI2026537, TESTI2026589, TESTI2026597, TESTI2026605, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034940, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723, TESTI3000002, TESTI3000005, TESTI4000014, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002072, TESTI4002141, TESTI4002195, TESTI4002202, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009286, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010851, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013474, TESTI4013602, TESTI4013667, TESTI4013675, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029.676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000017, THYMU1000025, THYMU1000029, THYMU1000032, THYMU1000033, THYMU1000041, THYMU1000060, THYMU1000074, THYMU1000083, THYMU1000098, THYMU1000103, THYMU1000105, THYMU1000109, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000329, THYMU1000331, THYMU1000336, THYMU1000359, THYMU1000366, THYMU1000382, THYMU1000393, THYMU1000394, THYMU1000399, THYMU1000426, THYMU1000440, THYMU1000459, THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000440, THYMU2000489, THYMU2000492, THYMU2000519, THYMU2000570, THYMU2000602, THYMU2000660, THYMU2000671, THYMU2000672, THYMU2000684, THYMU2000702, THYMU2000767, THYMU2000775, THYMU2000800, THYMU2000879, THYMU2000932, THYMU2000946, THYMU2000950, THYMU2000971, THYMU2000982, THYMU2000990, THYMU2001007, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001839, THYMU2001868, THYMU2001898, THYMU2001900, THYMU2001916, THYMU2001926, THYMU2001989, THYMU2002037, THYMU2002080, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002815, THYMU2002841, THYMU2002852, THYMU2002910, THYMU2002950, THYMU2002983, THYMU2003012, THYMU2003046, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003446, THYMU2003470, THYMU2003479, THYMU2003499, THYMU2003632, THYMU2003650, THYMU2003700, THYMU2003760, THYMU2003803, THYMU2003855, THYMU2003907, THYMU2003932, THYMU2003981, THYMU2004003, THYMU2004008, THYMU2004042, THYMU2004108, THYMU2004111, THYMU2004123, THYMU2004139, THYMU2004152, THYMU2004169, THYMU2004284, THYMU2004320, THYMU2004336, THYMU2004356, THYMU2004410, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006505, THYMU2006610, THYMU2006666, THYMU2006712, THYMU2006813, THYMU2006913, THYMU2006946, THYMU2006965, THYMU2007025, THYMU2007036, THYMU2007060, THYMU2007065, THYMU2007095, THYMU2007112, THYMU2007146, THYMU2007179, THYMU2007258, THYMU2007307, THYMU2007308, THYMU2007339, THYMU2007350, THYMU2007384, THYMU2007415, THYMU2007467, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007886, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010094, THYMU2010192, THYMU2010448, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012024, THYMU2012073, THYMU2012104, THYMU2012113, THYMU2012273, THYMU2012401, THYMU2012492, THYMU2012507, THYMU2012565, THYMU2012574, THYMU2012625, THYMU2012631, THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027125, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013386, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015759, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032798, THYMU3032867, THYMU3033402, THYMU3033626, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000044, TKIDN1000062, TKIDN1000066, TKIDN1000164, TKIDN1000171, TKIDN1000192, TKIDN2000127, TKIDN2000240, TKIDN2000246, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2003044, TKIDN2003059, TKIDN2003062, TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005487, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000030, TRACH1000038, TRACH1000057, TRACH1000074, TRACH1000100, TRACH1000106, TRACH1000125, TRACH1000140, TRACH1000144, TRACH1000193, TRACH1000212, TRACH1000240, TRACH2000002, TRACH2000015, TRACH2000024, TRACH2000032, TRACH2000045, TRACH2000047, TRACH2000081, TRACH2000083, TRACH2000148, TRACH2000185, TRACH2000196, TRACH2000235, TRACH2000237, TRACH2000243, TRACH2000250, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000420, TRACH2000461, TRACH2000496, TRACH2000497, TRACH2000502, TRACH2000522, TRACH2000540, TRACH2000559, TRACH2000660, TRACH2000665, TRACH2000675, TRACH2000677, TRACH2000702, TRACH2000703, TRACH2000767, TRACH2000775, TRACH2000780, TRACH2000807, TRACH2000862, TRACH2000873, TRACH2000894, TRACH2000898, TRACH2000920, TRACH2000923, TRACH2000926, TRACH2000944, TRACH2000959, TRACH2000981, TRACH2000986, TRACH2001021, TRACH2001037, TRACH2001060, TRACH2001085, TRACH2001101, TRACH2001109, TRACH2001121, TRACH2001147, TRACH2001154, TRACH2001188, TRACH2001192, TRACH2001249, TRACH2001275, TRACH2001314, TRACH2001335, TRACH2001339, TRACH2001341, TRACH2001347, TRACH2001395, TRACH2001400, TRACH2001403, TRACH2001416, TRACH2001428, TRACH2001443, TRACH2001474, TRACH2001478, TRACH2001525, TRACH2001532, TRACH2001549, TRACH2001592, TRACH2001596, TRACH2001612, TRACH2001621, TRACH2001646, TRACH2001651, TRACH2001684, TRACH2001810, TRACH2001898, TRACH2001933, TRACH2001968, TRACH2001996, TRACH2002043, TRACH2002054, TRACH2002100, TRACH2002138, TRACH2002664, TRACH2002784, TRACH2002803, TRACH2002840, TRACH2002847, TRACH2002853, TRACH2002862, TRACH2002954, TRACH2002960, TRACH2002988, TRACH2003070, TRACH2003272, TRACH2003347, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005800, TRACH2005811, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007754, TRACH2007816, TRACH2007894, TRACH2007988, TRACH2008081, TRACH2008112, TRACH2008113, TRACH2008127, TRACH2008130, TRACH2008278, TRACH2008300, TRACH2008472, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011113, TRACH2011290, TRACH2011293, TRACH2011302, TRACH2011500, TRACH2011574, TRACH2011894, TRACH2011956, TRACH2012000, TRACH2012138, TRACH2012242, TRACH2012298, TRACH2012370, TRACH2012376, TRACH2012387, TRACH2012536, TRACH2012562, TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014124, TRACH2014268, TRACH2014284, TRACH2014337, TRACH2014356, TRACH2014371, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016980, TRACH2017086, TRACH2017368, TRACH2017498, TRACH2017609, TRACH2017949, TRACH2017965, TRACH2017980, TRACH2018034, TRACH2018084, TRACH2018262, TRACH2018273, TRACH2018278, TRACH2018345, TRACH2018380, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002192, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003547, TRACH3003586, TRACH3003683, TRACH3003780, TRACH3003805, TRACH3003832, TRACH3003872, TRACH3004068, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007627, TRACH3007689, TRACH3007766, TRACH3007866, TRACH3007995, TRACH3008042, TRACH3008061, TRACH3008093, TRACH3008201, TRACH3008508, TRACH3008535, TRACH3008629, TRACH3008632, TRACH3008688, TRACH3008697, TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037067, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN1000186, UMVEN2000046, UMVEN2000069, UMVEN2000121, UMVEN2000152, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000182, UTERU1000183, UTERU1000187, UTERU1000192, UTERU1000249, UTERU1000337, UTERU1000339, UTERU1000384, UTERU2000023, UTERU2000047, UTERU2000074, UTERU2000099, UTERU2000154, UTERU2000197, UTERU2000218, UTERU2000238, UTERU2000243, UTERU2000260, UTERU2000300, UTERU2000329, UTERU2000332, UTERU2000338, UTERU2000349, UTERU2000377, UTERU2000393, UTERU2000418, UTERU2000465, UTERU2000485, UTERU2000517, UTERU2000524, UTERU2000537, UTERU2000539, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002198, UTERU2002294, UTERU2002332, UTERU2002410, UTERU2002473, UTERU2002547, UTERU2002693, UTERU2002733, UTERU2002736, UTERU2002737, UTERU2002826, UTERU2002841, UTERU2002964, UTERU2002993, UTERU2003035, UTERU2003057, UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004039, UTERU2004061, UTERU2004073, UTERU2004163, UTERU2004299, UTERU2004461, UTERU2004520, UTERU2004564, UTERU2004664, UTERU2004688, UTERU2004698, UTERU2004807, UTERU2004861, UTERU2004929, UTERU2005004, UTERU2005050, UTERU2005069, UTERU2005074, UTERU2005179, UTERU2005292, UTERU2005346, UTERU2005354, UTERU2005446, UTERU2005449, UTERU2005450, UTERU2005533, UTERU2005601, UTERU2005664, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547, UTERU2006568, UTERU2006593, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639, UTERU2007924, UTERU2007942, UTERU2008018, UTERU2008019, UTERU2008027, UTERU2008040, UTERU2008077, UTERU2008130, UTERU2008302, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008938, UTERU2008939, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009510, UTERU2009538, UTERU2009540, UTERU2009776, UTERU2009904, UTERU2009951, UTERU2009972, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012230, UTERU2012286, UTERU2012333, UTERU2012407, UTERU2012526, UTERU2012581, UTERU2012610, UTERU2012615, UTERU2012688, UTERU2012703, UTERU2012715, UTERU2012741, UTERU2012767, UTERU2012786, UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013926, UTERU2013976, UTERU2014001, UTERU2014024, UTERU2014167, UTERU2014223, UTERU2014398, UTERU2014464, UTERU2014548, UTERU2014601, UTERU2014631, UTERU2014668, UTERU2014678, UTERU2014728, UTERU2014898, UTERU2014998, UTERU2015087, UTERU2015190, UTERU2015198, UTERU2015202, UTERU2015405, UTERU2015640, UTERU2015830, UTERU2015880, UTERU2016147, UTERU2016157, UTERU2016426, UTERU2016464, UTERU2016669, UTERU2016757, UTERU2016761, UTERU2016799, UTERU2016822, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019005, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023175, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the BGGI1, BLADE, BNGH4, BRACE, BRALZ, BRAMY, and BRASW libraries were analyzed for expression frequency. The analysis results are described below. Each is a relative value, where the expression level over all tissues is taken as 100. The larger this value, the greater the level of expression.

TABLE 27

| Clone Name | BGGI1 | BLADE | BNGH4 | BRACE | BRALZ | BRAMY | BRASW |
|---|---|---|---|---|---|---|---|
| 3NB691000085 | 0 | 0 | 0 | 6.145 | 0 | 0 | 0 |
| 3NB691000116 | 0 | 0 | 0 | 0 | 0 | 1.639 | 0 |
| 3NB691000173 | 0 | 5.542 | 0 | 0 | 5.706 | 0 | 0 |
| 3NB692000281 | 0 | 0 | 0 | 0 | 0 | 0.444 | 0 |
| 3NB692000330 | 0 | 0 | 0 | 0.626 | 0 | 0 | 0 |
| 3NB692000374 | 0 | 0 | 0 | 2.309 | 3.919 | 2.185 | 0 |
| 3NB692000545 | 0 | 0 | 0 | 2.002 | 0 | 0 | 0 |
| 3NB692000912 | 0 | 0 | 0 | 1.667 | 0 | 0.947 | 0 |
| 3NB692001022 | 0 | 8.733 | 0 | 2.649 | 4.496 | 0 | 0 |
| 3NB692001288 | 0 | 0 | 0 | 2.127 | 10.827 | 3.019 | 0 |
| 3NB692001334 | 0 | 0 | 0 | 1.334 | 9.699 | 0.270 | 0 |
| 3NB692001442 | 0 | 0 | 0 | 0.857 | 0 | 0 | 0 |
| 3NB692001496 | 0 | 0 | 0 | 10.926 | 0 | 0 | 0 |
| 3NB692001719 | 0 | 0 | 0 | 9.777 | 0 | 0 | 0 |
| 3NB692001853 | 0 | 0 | 0 | 1.859 | 0 | 0 | 0 |
| 3NB692002051 | 0 | 0 | 0 | 6.063 | 0 | 0 | 0 |
| ADRGL1000002 | 0 | 0 | 0 | 3.506 | 0 | 1.659 | 0 |
| ADRGL1000018 | 0 | 0 | 0 | 17.162 | 0 | 0 | 0 |
| ADRGL1000033 | 0 | 0 | 29.445 | 0 | 0 | 0 | 0 |
| ADRGL1000065 | 0 | 0 | 0 | 4.003 | 0 | 5.682 | 0 |
| ADRGL2000006 | 0 | 0 | 0 | 4.427 | 0 | 0 | 0 |
| ADRGL2000042 | 0 | 0 | 19.585 | 7.210 | 0 | 5.118 | 0 |
| ADRGL2000074 | 0 | 0 | 0 | 0 | 0 | 14.813 | 0 |
| ADRGL2000097 | 0 | 0 | 0 | 0 | 0 | 2.240 | 0 |
| ADRGL2000328 | 0 | 0 | 0 | 9.556 | 0 | 0 | 0 |
| ADRGL2000644 | 0 | 0 | 0 | 0 | 38.408 | 0 | 0 |
| ADRGL2001830 | 0 | 0 | 0 | 8.483 | 0 | 0 | 0 |
| ADRGL2002013 | 0 | 0 | 0 | 19.155 | 0 | 0 | 0 |
| ADRGL2002191 | 0 | 0 | 0 | 0 | 0 | 12.881 | 0 |
| ADRGL2002477 | 0 | 0 | 0 | 0 | 0 | 1.714 | 0 |
| ADRGL2007313 | 0 | 0 | 0 | 6.955 | 0 | 0 | 0 |
| ADRGL2007906 | 0 | 0 | 0 | 0 | 0 | 9.081 | 0 |
| ADRGL2007974 | 0 | 0 | 0 | 0 | 0 | 14.813 | 0 |
| ADRGL2008966 | 0 | 0 | 0 | 0 | 0 | 14.813 | 0 |
| ADRGL2009146 | 0 | 0 | 0 | 10.913 | 0 | 0 | 0 |
| ADRGL2009273 | 0 | 0 | 0 | 1.972 | 0 | 0 | 0 |
| ADRGL2010152 | 0 | 0 | 0 | 8.223 | 0 | 0 | 0 |
| ADRGL2012038 | 0 | 0 | 0 | 1.459 | 0 | 0 | 0 |
| ASTRO2000014 | 0 | 0 | 0 | 0.291 | 0 | 0.828 | 0 |
| ASTRO2000046 | 0 | 0 | 0 | 0.579 | 0 | 3.289 | 0 |
| ASTRO2000141 | 0 | 0 | 0 | 0 | 0 | 3.060 | 0 |
| ASTRO2000381 | 0 | 0 | 48.430 | 0 | 0 | 0 | 0 |
| ASTRO2000480 | 0 | 0 | 0 | 8.038 | 0 | 0 | 0 |
| ASTRO2001029 | 0 | 0 | 0 | 0.890 | 0 | 2.527 | 0 |
| ASTRO2001107 | 0 | 0 | 0 | 5.184 | 0 | 14.720 | 0 |
| ASTRO2001806 | 0 | 20.083 | 0 | 2.031 | 0 | 0 | 0 |
| ASTRO2001823 | 0 | 0 | 0 | 2.061 | 0 | 2.925 | 0 |
| ASTRO2002024 | 0 | 0 | 0 | 0 | 0 | 13.210 | 0 |
| ASTRO2002459 | 0 | 0 | 0 | 0.621 | 0 | 0.882 | 0 |
| ASTRO2002693 | 0 | 49.753 | 0 | 0 | 0 | 0 | 0 |
| ASTRO2002733 | 0 | 0 | 0 | 0 | 0 | 31.003 | 0 |
| ASTRO2003024 | 0 | 0 | 0 | 0.673 | 3.425 | 2.865 | 0 |
| ASTRO2003212 | 0 | 0 | 0 | 10.632 | 0 | 0 | 0 |
| ASTRO2003316 | 0 | 0 | 0 | 2.094 | 0 | 0 | 0 |
| ASTRO2003461 | 0 | 0 | 0 | 4.228 | 0 | 0 | 0 |
| ASTRO2003840 | 0 | 0 | 47.769 | 4.396 | 0 | 6.241 | 0 |
| ASTRO2003960 | 0 | 0 | 0 | 17.003 | 0 | 0 | 0 |
| ASTRO2004032 | 0 | 0 | 0 | 4.326 | 0 | 0 | 0 |
| ASTRO2004177 | 0 | 0 | 0 | 17.003 | 0 | 0 | 0 |
| ASTRO2004974 | 0 | 0 | 0 | 2.758 | 0 | 1.958 | 0 |
| ASTRO2005423 | 0 | 0 | 0 | 4.888 | 0 | 6.939 | 0 |
| ASTRO2005553 | 0 | 0 | 0 | 0 | 0 | 2.611 | 0 |
| ASTRO2006475 | 0 | 0 | 0 | 0 | 0 | 7.505 | 0 |
| ASTRO2007047 | 0 | 0 | 0 | 4.450 | 0 | 2.106 | 0 |
| ASTRO2007948 | 0 | 0 | 0 | 3.555 | 0 | 2.523 | 0 |
| ASTRO2008216 | 0 | 0 | 0 | 0 | 0 | 3.215 | 0 |
| ASTRO2010819 | 0 | 0 | 0 | 6.686 | 0 | 3.390 | 0 |
| ASTRO2011422 | 0 | 0 | 0 | 5.149 | 0 | 0 | 0 |
| ASTRO2012552 | 0 | 0 | 0 | 1.049 | 0 | 0 | 0 |
| ASTRO2013585 | 0 | 3.422 | 3.759 | 0.346 | 0 | 0.491 | 0 |
| ASTRO2014392 | 0 | 0 | 0 | 0 | 0 | 14.891 | 0 |
| ASTRO2014498 | 0 | 0 | 0 | 38.064 | 0 | 0 | 0 |
| ASTRO2014561 | 0 | 0 | 0 | 0 | 0 | 8.880 | 0 |
| ASTRO2014576 | 0 | 0 | 0 | 0 | 0 | 11.388 | 0 |
| ASTRO2014863 | 0 | 23.712 | 0 | 2.398 | 0 | 0 | 0 |
| ASTRO2015328 | 0 | 0 | 0 | 2.701 | 0 | 3.835 | 0 |
| ASTRO2016491 | 0 | 0 | 0 | 0.357 | 0 | 2.030 | 0 |
| ASTRO2016819 | 0 | 0 | 0 | 4.580 | 0 | 0 | 0 |
| ASTRO2018169 | 0 | 0 | 0 | 0 | 0 | 0.952 | 0 |
| ASTRO2018540 | 0 | 0 | 0 | 0 | 0 | 1.567 | 0 |
| ASTRO2019171 | 0 | 0 | 0 | 38.064 | 0 | 0 | 0 |
| BGGI11000123 | 21.967 | 0 | 0 | 1.492 | 0 | 0.706 | 0 |
| BGGI11000193 | 93.562 | 0 | 0 | 0 | 0 | 0 | 0 |
| BGGI11000285 | 100 | 0 | 0 | 0 | 0 | 0 | 0 |
| BGGI12000022 | 100 | 0 | 0 | 0 | 0 | 0 | 0 |
| BGGI12000054 | 7.382 | 0 | 0 | 0.167 | 2.553 | 1.187 | 0 |
| BGGI12000067 | 100 | 0 | 0 | 0 | 0 | 0 | 0 |
| BGGI12000161 | 100 | 0 | 0 | 0 | 0 | 0 | 0 |
| BGGI12000533 | 70.764 | 0 | 0 | 0 | 0 | 0 | 0 |
| BGGI12000544 | 100 | 0 | 0 | 0 | 0 | 0 | 0 |
| BGGI12000616 | 28.025 | 0 | 0 | 1.269 | 0 | 0.901 | 0 |
| BGGI12000684 | 77.334 | 0 | 0 | 0 | 0 | 0 | 0 |
| BGGI12000693 | 59.168 | 0 | 0 | 0 | 0 | 0 | 0 |
| BGGI12000839 | 23.115 | 0 | 0 | 0 | 7.994 | 0 | 0 |
| BGGI12001002 | 55.645 | 0 | 0 | 0 | 6.414 | 0 | 0 |
| BGGI12001075 | 62.049 | 0 | 0 | 0 | 0 | 0 | 0 |
| BGGI12001097 | 35.225 | 0 | 17.333 | 0 | 0 | 1.132 | 0 |
| BGGI12001241 | 100 | 0 | 0 | 0 | 0 | 0 | 0 |
| BGGI12001247 | 100 | 0 | 0 | 0 | 0 | 0 | 0 |
| BGGI12001682 | 32.371 | 0 | 0 | 3.665 | 3.732 | 3.122 | 0 |
| BGGI12001714 | 79.939 | 0 | 0 | 0 | 0 | 0 | 0 |
| BLADE1000176 | 0 | 83.178 | 0 | 16.822 | 0 | 0 | 0 |
| BLADE2000181 | 0 | 100 | 0 | 0 | 0 | 0 | 0 |
| BLADE2000256 | 0 | 80.138 | 0 | 0 | 0 | 0 | 0 |
| BLADE2000340 | 0 | 100 | 0 | 0 | 0 | 0 | 0 |
| BLADE2000389 | 0 | 100 | 0 | 0 | 0 | 0 | 0 |
| BLADE2000463 | 0 | 100 | 0 | 0 | 0 | 0 | 0 |
| BLADE2000492 | 0 | 24.632 | 0 | 0 | 0 | 0 | 0 |
| BLADE2000579 | 0 | 100 | 0 | 0 | 0 | 0 | 0 |
| BLADE2000640 | 0 | 100 | 0 | 0 | 0 | 0 | 0 |
| BLADE2000866 | 0 | 100 | 0 | 0 | 0 | 0 | 0 |
| BLADE2000938 | 0 | 100 | 0 | 0 | 0 | 0 | 0 |
| BLADE2001031 | 0 | 100 | 0 | 0 | 0 | 0 | 0 |
| BLADE2001133 | 0 | 100 | 0 | 0 | 0 | 0 | 0 |
| BLADE2001138 | 0 | 100 | 0 | 0 | 0 | 0 | 0 |
| BLADE2001141 | 0 | 100 | 0 | 0 | 0 | 0 | 0 |
| BLADE2001371 | 0 | 87.309 | 0 | 0 | 0 | 0 | 0 |
| BLADE2001458 | 0 | 100 | 0 | 0 | 0 | 0 | 0 |
| BLADE2001575 | 0 | 66.545 | 0 | 0 | 0 | 0 | 0 |
| BLADE2001753 | 0 | 100 | 0 | 0 | 0 | 0 | 0 |
| BLADE2001935 | 0 | 100 | 0 | 0 | 0 | 0 | 0 |
| BLADE2001940 | 0 | 100 | 0 | 0 | 0 | 0 | 0 |
| BLADE2001987 | 0 | 100 | 0 | 0 | 0 | 0 | 0 |
| BLADE2002073 | 0 | 100 | 0 | 0 | 0 | 0 | 0 |
| BLADE2002310 | 0 | 100 | 0 | 0 | 0 | 0 | 0 |
| BLADE2002730 | 0 | 91.480 | 0 | 0 | 0 | 0 | 0 |
| BLADE2002738 | 0 | 86.356 | 0 | 0 | 0 | 0 | 0 |
| BLADE2002744 | 0 | 17.131 | 0 | 0 | 0 | 0 | 0 |
| BLADE2002782 | 0 | 100 | 0 | 0 | 0 | 0 | 0 |
| BLADE2002947 | 0 | 100 | 0 | 0 | 0 | 0 | 0 |
| BLADE2002972 | 0 | 100 | 0 | 0 | 0 | 0 | 0 |
| BLADE2003474 | 0 | 100 | 0 | 0 | 0 | 0 | 0 |
| BLADE2003916 | 0 | 100 | 0 | 0 | 0 | 0 | 0 |
| BLADE2004089 | 0 | 69.556 | 0 | 7.034 | 0 | 0 | 0 |
| BLADE2004345 | 0 | 100 | 0 | 0 | 0 | 0 | 0 |
| BLADE2004389 | 0 | 100 | 0 | 0 | 0 | 0 | 0 |
| BLADE2004462 | 0 | 100 | 0 | 0 | 0 | 0 | 0 |
| BLADE2004628 | 0 | 100 | 0 | 0 | 0 | 0 | 0 |
| BLADE2004670 | 0 | 100 | 0 | 0 | 0 | 0 | 0 |
| BLADE2004722 | 0 | 70.250 | 0 | 0 | 0 | 0 | 0 |
| BLADE2004849 | 0 | 52.024 | 0 | 0 | 0 | 0 | 0 |
| BLADE2005036 | 0 | 100 | 0 | 0 | 0 | 0 | 0 |
| BLADE2005038 | 0 | 100 | 0 | 0 | 0 | 0 | 0 |
| BLADE2005229 | 0 | 100 | 0 | 0 | 0 | 0 | 0 |
| BLADE2005459 | 0 | 100 | 0 | 0 | 0 | 0 | 0 |
| BLADE2005792 | 0 | 100 | 0 | 0 | 0 | 0 | 0 |
| BLADE2006043 | 0 | 100 | 0 | 0 | 0 | 0 | 0 |
| BLADE2006412 | 0 | 42.615 | 0 | 4.309 | 0 | 0 | 0 |
| BLADE2006415 | 0 | 100 | 0 | 0 | 0 | 0 | 0 |
| BLADE2006607 | 0 | 100 | 0 | 0 | 0 | 0 | 0 |
| BLADE2006826 | 0 | 100 | 0 | 0 | 0 | 0 | 0 |
| BLADE2006830 | 0 | 11.799 | 0 | 2.386 | 0 | 0 | 0 |
| BLADE2006849 | 0 | 100 | 0 | 0 | 0 | 0 | 0 |
| BLADE2006947 | 0 | 100 | 0 | 0 | 0 | 0 | 0 |
| BLADE2007033 | 0 | 100 | 0 | 0 | 0 | 0 | 0 |
| BLADE2007043 | 0 | 87.530 | 0 | 0 | 0 | 0 | 0 |
| BLADE2007589 | 0 | 34.697 | 0 | 0 | 0 | 0 | 0 |
| BLADE2007666 | 0 | 23.913 | 0 | 0 | 0 | 0 | 0 |
| BLADE2007722 | 0 | 100 | 0 | 0 | 0 | 0 | 0 |
| BLADE2007735 | 0 | 100 | 0 | 0 | 0 | 0 | 0 |
| BLADE2007744 | 0 | 100 | 0 | 0 | 0 | 0 | 0 |
| BLADE2007799 | 0 | 100 | 0 | 0 | 0 | 0 | 0 |
| BLADE2007923 | 0 | 100 | 0 | 0 | 0 | 0 | 0 |
| BLADE2007935 | 0 | 87.309 | 0 | 0 | 0 | 0 | 0 |
| BLADE2007958 | 0 | 100 | 0 | 0 | 0 | 0 | 0 |
| BLADE2008281 | 0 | 100 | 0 | 0 | 0 | 0 | 0 |
| BLADE2008398 | 0 | 43.522 | 0 | 4.401 | 0 | 0 | 0 |
| BLADE2008454 | 0 | 100 | 0 | 0 | 0 | 0 | 0 |
| BLADE2008461 | 0 | 17.399 | 0 | 0 | 0 | 2.498 | 0 |
| BLADE2008479 | 0 | 100 | 0 | 0 | 0 | 0 | 0 |
| BLADE2008539 | 0 | 100 | 0 | 0 | 0 | 0 | 0 |
| BLADE2008809 | 0 | 100 | 0 | 0 | 0 | 0 | 0 |
| BLADE2008995 | 0 | 100 | 0 | 0 | 0 | 0 | 0 |
| BLADE2009452 | 0 | 100 | 0 | 0 | 0 | 0 | 0 |
| BLADE2009479 | 0 | 100 | 0 | 0 | 0 | 0 | 0 |
| BLADE2009593 | 0 | 100 | 0 | 0 | 0 | 0 | 0 |
| BNGH41000098 | 0 | 0 | 19.548 | 0 | 0 | 2.554 | 0 |
| BNGH41000104 | 0 | 0 | 57.747 | 0 | 0 | 0 | 0 |
| BNGH41000118 | 0 | 0 | 100 | 0 | 0 | 0 | 0 |
| BNGH41000137 | 0 | 0 | 33.459 | 3.079 | 0 | 8.743 | 0 |
| BNGH41000153 | 0 | 0 | 36.238 | 0 | 0 | 4.735 | 0 |
| BNGH41000169 | 0 | 0 | 100 | 0 | 0 | 0 | 0 |
| BNGH41000177 | 0 | 0 | 53.617 | 0 | 0 | 0 | 0 |
| BNGH41000190 | 0 | 0 | 19.514 | 3.592 | 0 | 0 | 0 |
| BNGH41000198 | 0 | 0 | 16.022 | 0 | 0 | 2.093 | 0 |
| BNGH41000216 | 0 | 0 | 100 | 0 | 0 | 0 | 0 |
| BNGH41000235 | 0 | 0 | 50.263 | 0 | 0 | 0 | 0 |
| BNGH42000130 | 0 | 0 | 8.077 | 0 | 0 | 0 | 0 |
| BNGH42000132 | 0 | 0 | 100 | 0 | 0 | 0 | 0 |
| BNGH42000360 | 0 | 0 | 2.426 | 0.223 | 2.273 | 0.158 | 0 |
| BNGH42000405 | 0 | 0 | 100 | 0 | 0 | 0 | 0 |
| BNGH42000532 | 0 | 0 | 72.929 | 0 | 0 | 0 | 0 |
| BNGH42000675 | 0 | 0 | 100 | 0 | 0 | 0 | 0 |
| BNGH42000739 | 0 | 0 | 4.798 | 10.598 | 2.248 | 6.896 | 0 |
| BNGH42000791 | 0 | 0 | 100 | 0 | 0 | 0 | 0 |
| BNGH42000815 | 0 | 0 | 67.530 | 0 | 0 | 0 | 0 |
| BNGH42000994 | 0 | 0 | 100 | 0 | 0 | 0 | 0 |
| BNGH42001247 | 0 | 0 | 40.570 | 3.734 | 0 | 0 | 0 |
| BNGH42001406 | 0 | 0 | 52.989 | 0 | 0 | 0 | 0 |
| BNGH42001576 | 0 | 0 | 37.628 | 0 | 0 | 0 | 0 |
| BNGH42001724 | 0 | 0 | 100 | 0 | 0 | 0 | 0 |
| BNGH42002168 | 0 | 9.339 | 10.261 | 0.944 | 0 | 1.341 | 0 |
| BNGH42002244 | 0 | 0 | 100 | 0 | 0 | 0 | 0 |
| BNGH42002387 | 0 | 0 | 81.396 | 0 | 0 | 0 | 0 |
| BNGH42002487 | 0 | 0 | 72.180 | 0 | 0 | 0 | 0 |
| BNGH42002489 | 0 | 0 | 100 | 0 | 0 | 0 | 0 |
| BNGH42003529 | 0 | 0 | 100 | 0 | 0 | 0 | 0 |
| BNGH42003570 | 0 | 0 | 100 | 0 | 0 | 0 | 0 |
| BNGH42003641 | 0 | 0 | 100 | 0 | 0 | 0 | 0 |
| BNGH42004076 | 0 | 0 | 100 | 0 | 0 | 0 | 0 |
| BNGH42004291 | 0 | 0 | 100 | 0 | 0 | 0 | 0 |
| BNGH42004538 | 0 | 0 | 100 | 0 | 0 | 0 | 0 |
| BNGH42004679 | 0 | 0 | 92.186 | 0 | 0 | 0 | 0 |
| BNGH42005017 | 0 | 0 | 67.530 | 0 | 0 | 0 | 0 |
| BNGH42005235 | 0 | 0 | 100 | 0 | 0 | 0 | 0 |
| BNGH42005504 | 0 | 0 | 100 | 0 | 0 | 0 | 0 |
| BNGH42005968 | 0 | 0 | 17.713 | 0 | 0 | 0 | 0 |
| BNGH42006135 | 0 | 0 | 5.091 | 3.749 | 0 | 2.661 | 0 |
| BNGH42006226 | 0 | 0 | 88.444 | 0 | 0 | 11.556 | 0 |
| BNGH42006234 | 0 | 0 | 100 | 0 | 0 | 0 | 0 |
| BNGH42007037 | 0 | 0 | 95.934 | 0 | 0 | 0 | 0 |
| BNGH42007366 | 0 | 0 | 49.317 | 0 | 0 | 0 | 0 |
| BNGH42007460 | 31.586 | 0 | 7.771 | 2.861 | 0 | 3.046 | 0 |
| BNGH42007594 | 0 | 0 | 100 | 0 | 0 | 0 | 0 |
| BNGH42007709 | 7.215 | 1.616 | 1.775 | 0 | 3.327 | 0.464 | 0 |
| BNGH42007788 | 0 | 0 | 23.740 | 0 | 0 | 0 | 0 |
| BNGH42007798 | 0 | 0 | 75.734 | 0 | 0 | 0 | 0 |
| BNGH42008199 | 0 | 0 | 100 | 0 | 0 | 0 | 0 |
| BNGH42008510 | 0 | 0 | 100 | 0 | 0 | 0 | 0 |
| BNGH42008603 | 0 | 0 | 11.154 | 3.080 | 0 | 5.829 | 0 |
| BNGH42008649 | 0 | 0 | 100 | 0 | 0 | 0 | 0 |
| BNGH42008743 | 0 | 0 | 11.872 | 2.185 | 0 | 0 | 0 |
| BNGH42008832 | 0 | 0 | 100 | 0 | 0 | 0 | 0 |
| BNGH42008850 | 0 | 0 | 81.250 | 0 | 0 | 0 | 0 |
| BNGH42008943 | 0 | 0 | 64.079 | 5.898 | 30.024 | 0 | 0 |
| BNGH42009021 | 0 | 0 | 100 | 0 | 0 | 0 | 0 |
| BNGH42009025 | 0 | 0 | 10.751 | 3.958 | 0 | 1.405 | 0 |
| BRACE1000020 | 0 | 0 | 0 | 7.989 | 0 | 0 | 0 |
| BRACE1000042 | 0 | 0 | 0 | 5.402 | 0 | 6.135 | 0 |
| BRACE1000047 | 0 | 0 | 0 | 100 | 0 | 0 | 0 |
| BRACE1000051 | 0 | 0 | 0 | 50.891 | 0 | 0 | 0 |
| BRACE1000065 | 0 | 0 | 0 | 56.141 | 0 | 0 | 0 |
| BRACE1000070 | 0 | 0 | 0 | 1.269 | 0 | 0 | 0 |
| BRACE1000073 | 0 | 0 | 0 | 5.985 | 15.235 | 0 | 0 |
| BRACE1000084 | 0 | 0 | 0 | 100 | 0 | 0 | 0 |
| BRACE1000087 | 0 | 0 | 0 | 10.415 | 0 | 0 | 0 |
| BRACE1000092 | 0 | 0 | 0 | 100 | 0 | 0 | 0 |
| BRACE1000093 | 0 | 0 | 0 | 27.755 | 0 | 0 | 0 |
| BRACE1000101 | 0 | 0 | 0 | 7.942 | 40.434 | 11.275 | 0 |
| BRACE1000114 | 0 | 0 | 0 | 100 | 0 | 0 | 0 |
| BRACE1000115 | 0 | 0 | 0 | 2.790 | 6.764 | 0.754 | 70.707 |
| BRACE1000124 | 0 | 0 | 0 | 100 | 0 | 0 | 0 |
| BRACE1000131 | 0 | 0 | 0 | 1.479 | 0 | 0 | 0 |
| BRACE1000132 | 0 | 0 | 0 | 10.195 | 0 | 14.473 | 0 |
| BRACE1000159 | 0 | 0 | 0 | 5.242 | 13.343 | 0 | 0 |
| BRACE1000166 | 0 | 0 | 0 | 45.981 | 0 | 0 | 0 |
| BRACE1000169 | 0 | 0 | 0 | 28.778 | 0 | 0 | 0 |
| BRACE1000186 | 0 | 0 | 0 | 1.535 | 0 | 0 | 0 |
| BRACE1000187 | 0 | 0 | 0 | 9.578 | 9.752 | 0 | 0 |
| BRACE1000239 | 0 | 0 | 0 | 28.128 | 0 | 0 | 0 |
| BRACE1000258 | 0 | 0 | 0 | 68.044 | 0 | 0 | 0 |
| BRACE1000451 | 0 | 0 | 0 | 100 | 0 | 0 | 0 |
| BRACE1000475 | 0 | 0 | 0 | 100 | 0 | 0 | 0 |
| BRACE1000572 | 0 | 0 | 0 | 100 | 0 | 0 | 0 |
| BRACE2000061 | 0 | 0 | 0 | 27.821 | 0 | 39.495 | 0 |
| BRACE2000077 | 0 | 0 | 0 | 68.470 | 0 | 0 | 0 |
| BRACE2000078 | 0 | 0 | 0 | 3.177 | 0 | 0 | 0 |
| BRACE2000100 | 0 | 0 | 0 | 100 | 0 | 0 | 0 |
| BRACE2000109 | 0 | 0 | 0 | 39.025 | 0 | 0 | 0 |
| BRACE2000141 | 0 | 0 | 0 | 12.327 | 0 | 17.500 | 0 |
| BRACE2000148 | 0 | 0 | 0 | 2.534 | 0 | 0 | 0 |
| BRACE2000183 | 0 | 0 | 0 | 3.254 | 0 | 0 | 0 |
| BRACE2000229 | 0 | 0 | 0 | 9.165 | 0 | 0 | 0 |
| BRACE2000232 | 0 | 0 | 0 | 100 | 0 | 0 | 0 |
| BRACE2000245 | 0 | 0 | 0 | 6.277 | 0 | 8.911 | 0 |
| BRACE2000256 | 0 | 0 | 0 | 100 | 0 | 0 | 0 |
| BRACE2000266 | 0 | 0 | 0 | 100 | 0 | 0 | 0 |
| BRACE2000267 | 0 | 0 | 0 | 28.458 | 48.292 | 0 | 0 |
| BRACE2000280 | 0 | 0 | 0 | 13.302 | 0 | 0 | 0 |
| BRACE2000300 | 0 | 0 | 0 | 3.773 | 0 | 5.356 | 0 |
| BRACE2000307 | 0 | 0 | 0 | 20.064 | 0 | 0 | 0 |
| BRACE2000331 | 0 | 0 | 0 | 41.512 | 0 | 0 | 0 |
| BRACE2000332 | 0 | 19.052 | 0 | 3.853 | 0 | 0 | 0 |
| BRACE2000347 | 0 | 0 | 0 | 3.670 | 0 | 2.605 | 0 |
| BRACE2000351 | 0 | 0 | 0 | 100 | 0 | 0 | 0 |
| BRACE2000368 | 0 | 0 | 0 | 100 | 0 | 0 | 0 |
| BRACE2000392 | 0 | 0 | 0 | 100 | 0 | 0 | 0 |
| BRACE2000441 | 0 | 0 | 0 | 100 | 0 | 0 | 0 |
| BRACE2000487 | 0 | 57.595 | 0 | 5.824 | 0 | 0 | 0 |
| BRACE2000489 | 0 | 0 | 0 | 100 | 0 | 0 | 0 |
| BRACE2000505 | 0 | 0 | 0 | 8.673 | 0 | 0 | 0 |
| BRACE2000525 | 0 | 0 | 0 | 8.072 | 5.871 | 1.637 | 0 |
| BRACE2000526 | 0 | 0 | 0 | 100 | 0 | 0 | 0 |
| BRACE2000531 | 0 | 0 | 0 | 100 | 0 | 0 | 0 |
| BRACE2000545 | 0 | 0 | 0 | 100 | 0 | 0 | 0 |
| BRACE2000553 | 0 | 0 | 0 | 100 | 0 | 0 | 0 |
| BRACE2000565 | 0 | 0 | 0 | 15.271 | 0 | 7.226 | 0 |
| BRACE2000577 | 0 | 0 | 0 | 52.057 | 0 | 0 | 0 |
| BRACE2000640 | 0 | 0 | 0 | 100 | 0 | 0 | 0 |
| BRACE2000650 | 0 | 0 | 0 | 100 | 0 | 0 | 0 |
| BRACE2000677 | 0 | 0 | 0 | 100 | 0 | 0 | 0 |
| BRACE2000698 | 0 | 0 | 0 | 6.107 | 0 | 0 | 0 |
| BRACE2000718 | 0 | 0 | 0 | 100 | 0 | 0 | 0 |
| BRACE2000733 | 0 | 0 | 0 | 12.509 | 0 | 0 | 0 |
| BRACE2000743 | 0 | 0 | 0 | 100 | 0 | 0 | 0 |
| BRACE2000750 | 0 | 0 | 0 | 14.608 | 0 | 0 | 0 |
| BRACE2000753 | 0 | 0 | 0 | 21.618 | 0 | 0 | 0 |
| BRACE2000815 | 0 | 0 | 0 | 100 | 0 | 0 | 0 |
| BRACE2000864 | 0 | 0 | 0 | 2.997 | 0 | 4.254 | 0 |
| BRACE2000885 | 0 | 0 | 0 | 52.057 | 0 | 0 | 0 |
| BRACE2000893 | 0 | 0 | 0 | 35.283 | 0 | 0 | 0 |
| BRACE2000905 | 0 | 0 | 0 | 100 | 0 | 0 | 0 |
| BRACE2000936 | 0 | 0 | 0 | 100 | 0 | 0 | 0 |
| BRACE2000947 | 0 | 0 | 0 | 100 | 0 | 0 | 0 |
| BRACE2000965 | 0 | 0 | 0 | 100 | 0 | 0 | 0 |
| BRACE2000969 | 0 | 0 | 0 | 16.214 | 0 | 0 | 0 |
| BRACE2000975 | 0 | 0 | 0 | 15.004 | 0 | 7.100 | 0 |
| BRACE2000988 | 0 | 0 | 0 | 100 | 0 | 0 | 0 |
| BRACE2001017 | 0 | 0 | 0 | 100 | 0 | 0 | 0 |
| BRACE2001065 | 0 | 0 | 0 | 4.713 | 47.986 | 0 | 0 |
| BRACE2001070 | 0 | 0 | 0 | 16.015 | 0 | 0 | 0 |
| BRACE2001094 | 0 | 0 | 0 | 100 | 0 | 0 | 0 |
| BRACE2001107 | 0 | 0 | 0 | 10.447 | 53.184 | 0 | 0 |
| BRACE2001117 | 0 | 0 | 0 | 3.904 | 9.938 | 0 | 0 |
| BRACE2001143 | 0 | 0 | 0 | 3.163 | 0 | 2.994 | 0 |
| BRACE2001151 | 0 | 0 | 0 | 4.581 | 0 | 0 | 0 |
| BRACE2001154 | 0 | 0 | 0 | 7.541 | 0 | 0 | 0 |
| BRACE2001188 | 0 | 19.813 | 0 | 2.004 | 10.200 | 0 | 0 |
| BRACE2001191 | 0 | 0 | 0 | 100 | 0 | 0 | 0 |
| BRACE2001197 | 0 | 0 | 0 | 41.027 | 0 | 0 | 0 |
| BRACE2001236 | 0 | 0 | 0 | 12.771 | 0 | 0 | 0 |
| BRACE2001239 | 0 | 0 | 0 | 100 | 0 | 0 | 0 |
| BRACE2001269 | 0 | 0 | 0 | 100 | 0 | 0 | 0 |
| BRACE2001312 | 0 | 0 | 0 | 100 | 0 | 0 | 0 |
| BRACE2001313 | 0 | 0 | 0 | 21.618 | 0 | 0 | 0 |
| BRACE2001330 | 0 | 0 | 0 | 100 | 0 | 0 | 0 |
| BRACE2001340 | 0 | 0 | 0 | 100 | 0 | 0 | 0 |
| BRACE2001352 | 0 | 0 | 0 | 100 | 0 | 0 | 0 |
| BRACE2001353 | 0 | 0 | 0 | 100 | 0 | 0 | 0 |
| BRACE2001374 | 0 | 0 | 0 | 41.329 | 0 | 58.671 | 0 |
| BRACE2001375 | 0 | 0 | 0 | 100 | 0 | 0 | 0 |
| BRACE2001423 | 0 | 0 | 0 | 100 | 0 | 0 | 0 |
| BRACE2001424 | 0 | 0 | 0 | 100 | 0 | 0 | 0 |
| BRACE2001445 | 0 | 0 | 0 | 100 | 0 | 0 | 0 |
| BRACE2001453 | 0 | 0 | 0 | 100 | 0 | 0 | 0 |
| BRACE2001455 | 0 | 0 | 0 | 100 | 0 | 0 | 0 |
| BRACE2001477 | 0 | 0 | 0 | 4.882 | 0 | 6.931 | 0 |
| BRACE2001492 | 0 | 0 | 0 | 100 | 0 | 0 | 0 |
| BRACE2001496 | 0 | 0 | 0 | 6.887 | 17.530 | 4.888 | 0 |
| BRACE2001497 | 0 | 0 | 0 | 2.645 | 0 | 3.755 | 0 |
| BRACE2001508 | 0 | 0 | 0 | 12.511 | 0 | 0 | 0 |
| BRACE2001534 | 0 | 0 | 0 | 4.379 | 0 | 0 | 0 |
| BRACE2001543 | 0 | 0 | 0 | 100 | 0 | 0 | 0 |
| BRACE2001564 | 0 | 0 | 0 | 68.470 | 0 | 0 | 0 |
| BRACE2001588 | 0 | 0 | 0 | 100 | 0 | 0 | 0 |
| BRACE2001607 | 0 | 0 | 0 | 100 | 0 | 0 | 0 |
| BRACE2001626 | 0 | 0 | 0 | 100 | 0 | 0 | 0 |
| BRACE2001673 | 0 | 0 | 0 | 1.498 | 2.541 | 0.236 | 88.558 |
| BRACE2001677 | 0 | 0 | 0 | 100 | 0 | 0 | 0 |
| BRACE2001692 | 0 | 0 | 0 | 0.860 | 0 | 0 | 0 |
| BRACE2001705 | 0 | 0 | 0 | 100 | 0 | 0 | 0 |
| BRACE2001709 | 0 | 0 | 0 | 4.607 | 11.727 | 0 | 0 |
| BRACE2001711 | 0 | 0 | 0 | 8.283 | 0 | 0 | 0 |
| BRACE2001726 | 0 | 0 | 0 | 25.113 | 0 | 35.650 | 0 |
| BRACE2001733 | 0 | 0 | 0 | 100 | 0 | 0 | 0 |
| BRACE2001737 | 0 | 0 | 0 | 100 | 0 | 0 | 0 |
| BRACE2001742 | 0 | 0 | 0 | 100 | 0 | 0 | 0 |
| BRACE2001777 | 0 | 0 | 0 | 100 | 0 | 0 | 0 |
| BRACE2001779 | 0 | 0 | 0 | 100 | 0 | 0 | 0 |
| BRACE2001834 | 0 | 0 | 0 | 10.775 | 0 | 7.648 | 0 |
| BRACE2001855 | 0 | 0 | 0 | 0.817 | 0 | 0 | 0 |
| BRACE2001859 | 0 | 0 | 0 | 9.658 | 0 | 0 | 0 |
| BRACE2001865 | 0 | 0 | 0 | 100 | 0 | 0 | 0 |
| BRACE2001872 | 0 | 0 | 0 | 100 | 0 | 0 | 0 |
| BRACE2001881 | 0 | 0 | 0 | 16.377 | 0 | 0 | 0 |
| BRACE2001892 | 0 | 0 | 0 | 7.525 | 1.095 | 8.546 | 0 |
| BRACE2001898 | 0 | 0 | 0 | 100 | 0 | 0 | 0 |
| BRACE2001923 | 0 | 0 | 0 | 100 | 0 | 0 | 0 |
| BRACE2001942 | 0 | 0 | 0 | 45.981 | 0 | 0 | 0 |
| BRACE2001944 | 0 | 0 | 0 | 8.905 | 22.667 | 0 | 0 |
| BRACE2001954 | 0 | 0 | 0 | 100 | 0 | 0 | 0 |
| BRACE2001971 | 0 | 0 | 17.297 | 4.776 | 8.105 | 0 | 0 |
| BRACE2001972 | 0 | 0 | 0 | 100 | 0 | 0 | 0 |
| BRACE2001973 | 0 | 0 | 0 | 100 | 0 | 0 | 0 |
| BRACE2002026 | 0 | 0 | 0 | 100 | 0 | 0 | 0 |
| BRACE2002034 | 0 | 0 | 0 | 100 | 0 | 0 | 0 |
| BRACE2002050 | 0 | 0 | 0 | 14.407 | 0 | 14.609 | 0 |
| BRACE2002081 | 0 | 0 | 0 | 13.619 | 0 | 0 | 0 |
| BRACE2002091 | 0 | 0 | 0 | 1.499 | 0 | 2.128 | 0 |
| BRACE2002139 | 0 | 0 | 0 | 100 | 0 | 0 | 0 |
| BRACE2002142 | 0 | 0 | 0 | 100 | 0 | 0 | 0 |
| BRACE2002151 | 0 | 0 | 0 | 100 | 0 | 0 | 0 |
| BRACE2002157 | 0 | 0 | 0 | 100 | 0 | 0 | 0 |
| BRACE2002176 | 0 | 0 | 0 | 37.308 | 0 | 0 | 0 |
| BRACE2002202 | 0 | 0 | 0 | 76.511 | 0 | 0 | 0 |
| BRACE2002206 | 0 | 0 | 0 | 100 | 0 | 0 | 0 |
| BRACE2002227 | 0 | 0 | 0 | 100 | 0 | 0 | 0 |
| BRACE2002260 | 0 | 0 | 0 | 100 | 0 | 0 | 0 |
| BRACE2002297 | 0 | 0 | 0 | 100 | 0 | 0 | 0 |
| BRACE2002304 | 0 | 0 | 0 | 100 | 0 | 0 | 0 |
| BRACE2002356 | 0 | 0 | 0 | 25.793 | 0 | 0 | 0 |
| BRACE2002392 | 0 | 0 | 0 | 3.670 | 18.684 | 5.210 | 0 |
| BRACE2002394 | 0 | 0 | 0 | 100 | 0 | 0 | 0 |
| BRACE2002409 | 0 | 0 | 0 | 37.308 | 0 | 0 | 0 |
| BRACE2002431 | 0 | 0 | 0 | 100 | 0 | 0 | 0 |
| BRACE2002441 | 0 | 0 | 0 | 0.799 | 0 | 4.539 | 0 |
| BRACE2002444 | 0 | 0 | 0 | 100 | 0 | 0 | 0 |
| BRACE2002458 | 0 | 0 | 0 | 100 | 0 | 0 | 0 |
| BRACE2002467 | 0 | 0 | 0 | 4.639 | 0 | 0 | 0 |
| BRACE2002468 | 0 | 0 | 0 | 100 | 0 | 0 | 0 |
| BRACE2002478 | 0 | 0 | 0 | 41.149 | 0 | 0 | 0 |
| BRACE2002495 | 0 | 0 | 0 | 16.447 | 0 | 0 | 0 |
| BRACE2002582 | 0 | 0 | 0 | 9.274 | 0 | 0 | 0 |
| BRACE2002585 | 0 | 0 | 0 | 17.693 | 0 | 25.117 | 0 |
| BRACE2002589 | 0 | 0 | 0 | 100 | 0 | 0 | 0 |
| BRACE2002590 | 0 | 0 | 0 | 100 | 0 | 0 | 0 |
| BRACE2002606 | 0 | 0 | 0 | 3.804 | 0 | 0 | 0 |
| BRACE2002613 | 0 | 0 | 0 | 100 | 0 | 0 | 0 |
| BRACE2002614 | 0 | 0 | 0 | 100 | 0 | 0 | 0 |
| BRACE2002617 | 0 | 0 | 0 | 100 | 0 | 0 | 0 |
| BRACE2002623 | 0 | 0 | 0 | 100 | 0 | 0 | 0 |
| BRACE2002635 | 0 | 0 | 0 | 41.329 | 0 | 58.671 | 0 |
| BRACE2002649 | 0 | 0 | 0 | 15.030 | 0 | 0 | 0 |
| BRACE2002661 | 0 | 0 | 0 | 2.044 | 0 | 0 | 0 |
| BRACE2002676 | 0 | 0 | 0 | 100 | 0 | 0 | 0 |
| BRACE2002677 | 0 | 0 | 0 | 1.370 | 0 | 0 | 0 |
| BRACE2002682 | 0 | 0 | 0 | 13.590 | 0 | 0 | 0 |
| BRACE2002685 | 0 | 0 | 0 | 2.433 | 0 | 0 | 0 |
| BRACE2002695 | 0 | 0 | 0 | 100 | 0 | 0 | 0 |
| BRACE2002707 | 0 | 0 | 0 | 100 | 0 | 0 | 0 |
| BRACE2002736 | 0 | 0 | 0 | 100 | 0 | 0 | 0 |
| BRACE2002752 | 0 | 0 | 0 | 10.913 | 0 | 0 | 0 |
| BRACE2002755 | 0 | 0 | 0 | 38.787 | 0 | 0 | 0 |
| BRACE2002762 | 0 | 0 | 0 | 100 | 0 | 0 | 0 |
| BRACE2002772 | 0 | 0 | 0 | 100 | 0 | 0 | 0 |
| BRACE2002792 | 0 | 0 | 0 | 100 | 0 | 0 | 0 |
| BRACE2002796 | 0 | 0 | 0 | 36.360 | 0 | 0 | 0 |
| BRACE2002803 | 0 | 0 | 0 | 38.798 | 0 | 18.360 | 0 |
| BRACE2002807 | 0 | 0 | 0 | 22.873 | 0 | 0 | 0 |
| BRACE2002812 | 0 | 0 | 0 | 25.026 | 0 | 5.921 | 0 |
| BRACE2002834 | 0 | 0 | 0 | 100 | 0 | 0 | 0 |
| BRACE2002849 | 0 | 0 | 0 | 100 | 0 | 0 | 0 |
| BRACE2002857 | 0 | 0 | 0 | 100 | 0 | 0 | 0 |
| BRACE2002860 | 0 | 0 | 0 | 7.148 | 0 | 0 | 0 |
| BRACE2002861 | 0 | 18.215 | 0 | 9.209 | 0 | 0 | 0 |
| BRACE2002884 | 0 | 0 | 0 | 100 | 0 | 0 | 0 |
| BRACE2002896 | 0 | 0 | 0 | 31.914 | 0 | 0 | 0 |
| BRACE2002948 | 0 | 0 | 0 | 100 | 0 | 0 | 0 |
| BRACE2003037 | 0 | 0 | 0 | 3.065 | 23.406 | 10.878 | 0 |
| BRACE2003078 | 0 | 0 | 0 | 11.488 | 0 | 0 | 0 |
| BRACE2003097 | 0 | 0 | 0 | 41.027 | 0 | 0 | 0 |
| BRACE2003110 | 0 | 0 | 0 | 9.561 | 0 | 0 | 0 |
| BRACE2003168 | 0 | 0 | 0 | 100 | 0 | 0 | 0 |
| BRACE2003172 | 0 | 0 | 0 | 5.536 | 2.349 | 1.637 | 0 |
| BRACE2003199 | 0 | 0 | 0 | 100 | 0 | 0 | 0 |
| BRACE2003210 | 0 | 0 | 0 | 100 | 0 | 0 | 0 |
| BRACE2003217 | 0 | 0 | 0 | 6.119 | 0 | 8.686 | 0 |
| BRACE2003254 | 0 | 6.355 | 0 | 1.928 | 0 | 2.737 | 0 |
| BRACE2003269 | 0 | 0 | 0 | 100 | 0 | 0 | 0 |
| BRACE2003285 | 0 | 0 | 0 | 100 | 0 | 0 | 0 |
| BRACE2003304 | 0 | 0 | 0 | 3.103 | 0 | 0 | 0 |
| BRACE2003319 | 0 | 0 | 0 | 1.626 | 0 | 0 | 0 |
| BRACE2003398 | 0 | 0 | 0 | 16.418 | 83.582 | 0 | 0 |
| BRACE2003420 | 0 | 0 | 0 | 100 | 0 | 0 | 0 |
| BRACE2003428 | 0 | 0 | 0 | 100 | 0 | 0 | 0 |
| BRACE2003431 | 0 | 0 | 0 | 100 | 0 | 0 | 0 |
| BRACE2003512 | 0 | 0 | 0 | 22.932 | 0 | 0 | 0 |
| BRACE2003516 | 0 | 0 | 0 | 37.308 | 0 | 0 | 0 |
| BRACE2003527 | 0 | 0 | 0 | 3.659 | 0 | 0 | 0 |
| BRACE2003539 | 0 | 0 | 0 | 39.025 | 0 | 0 | 0 |
| BRACE2003540 | 0 | 0 | 0 | 100 | 0 | 0 | 0 |
| BRACE2003594 | 0 | 0 | 0 | 8.288 | 0 | 0 | 0 |
| BRACE2003598 | 0 | 0 | 0 | 39.025 | 0 | 0 | 0 |
| BRACE2003609 | 0 | 0 | 0 | 6.452 | 0 | 18.317 | 0 |
| BRACE2003614 | 0 | 0 | 0 | 100 | 0 | 0 | 0 |
| BRACE2003628 | 0 | 0 | 0 | 9.684 | 0 | 0 | 0 |
| BRACE2003639 | 0 | 0 | 0 | 100 | 0 | 0 | 0 |
| BRACE2003700 | 0 | 0 | 0 | 29.243 | 0 | 41.515 | 0 |
| BRACE2003713 | 0 | 0 | 0 | 16.418 | 83.582 | 0 | 0 |
| BRACE2003766 | 0 | 0 | 0 | 100 | 0 | 0 | 0 |
| BRACE2003785 | 0 | 0 | 0 | 6.207 | 0 | 10.770 | 0 |
| BRACE2003788 | 0 | 0 | 0 | 27.684 | 0 | 0 | 0 |
| BRACE2003800 | 0 | 0 | 0 | 38.906 | 0 | 0 | 0 |
| BRACE2003802 | 0 | 0 | 0 | 100 | 0 | 0 | 0 |
| BRACE2003825 | 0 | 0 | 0 | 9.658 | 0 | 0 | 0 |
| BRACE2003827 | 0 | 0 | 0 | 5.597 | 3.166 | 0 | 0 |
| BRACE2003845 | 0 | 0 | 0 | 6.468 | 0 | 0 | 0 |
| BRACE2003846 | 0 | 0 | 0 | 1.850 | 0 | 0.657 | 0 |
| BRACE2003847 | 0 | 0 | 0 | 100 | 0 | 0 | 0 |
| BRACE2003848 | 0 | 0 | 0 | 38.787 | 0 | 0 | 0 |
| BRACE2003873 | 0 | 0 | 0 | 100 | 0 | 0 | 0 |
| BRACE2003885 | 0 | 0 | 0 | 100 | 0 | 0 | 0 |
| BRACE2003887 | 0 | 0 | 0 | 100 | 0 | 0 | 0 |
| BRACE2003892 | 0 | 0 | 0 | 6.196 | 0 | 6.597 | 0 |
| BRACE2003904 | 0 | 0 | 0 | 3.680 | 9.367 | 0 | 0 |
| BRACE2003905 | 0 | 0 | 0 | 44.896 | 0 | 0 | 0 |
| BRACE2003928 | 0 | 0 | 0 | 100 | 0 | 0 | 0 |
| BRACE2003944 | 0 | 0 | 0 | 0.367 | 0 | 2.086 | 0 |
| BRACE2003954 | 0 | 0 | 0 | 16.746 | 0 | 0 | 0 |
| BRACE2003982 | 0 | 0 | 0 | 2.921 | 14.870 | 8.293 | 0 |
| BRACE2003987 | 0 | 0 | 0 | 100 | 0 | 0 | 0 |
| BRACE2005034 | 0 | 0 | 0 | 100 | 0 | 0 | 0 |
| BRACE2005047 | 0 | 0 | 0 | 100 | 0 | 0 | 0 |
| BRACE2005063 | 0 | 0 | 0 | 100 | 0 | 0 | 0 |
| BRACE2005083 | 0 | 0 | 0 | 51.220 | 0 | 24.238 | 0 |
| BRACE2005087 | 0 | 0 | 0 | 6.626 | 0 | 3.136 | 0 |
| BRACE2005090 | 0 | 0 | 0 | 52.057 | 0 | 0 | 0 |
| BRACE2005099 | 0 | 0 | 0 | 14.152 | 0 | 1.339 | 0 |
| BRACE2005118 | 0 | 0 | 0 | 100 | 0 | 0 | 0 |
| BRACE2005124 | 0 | 0 | 0 | 100 | 0 | 0 | 0 |
| BRACE2005138 | 0 | 0 | 0 | 100 | 0 | 0 | 0 |
| BRACE2005151 | 0 | 0 | 0 | 7.457 | 0 | 0 | 0 |
| BRACE2005160 | 0 | 0 | 0 | 100 | 0 | 0 | 0 |
| BRACE2005169 | 0 | 0 | 0 | 100 | 0 | 0 | 0 |
| BRACE2005189 | 0 | 0 | 0 | 53.953 | 0 | 0 | 0 |
| BRACE2005193 | 0 | 0 | 0 | 41.027 | 0 | 0 | 0 |
| BRACE2005196 | 0 | 0 | 0 | 22.570 | 0 | 0 | 0 |
| BRACE2005198 | 0 | 0 | 0 | 100 | 0 | 0 | 0 |
| BRACE2005216 | 0 | 0 | 0 | 100 | 0 | 0 | 0 |
| BRACE2005231 | 0 | 22.408 | 0 | 9.064 | 11.536 | 9.650 | 0 |
| BRACE2005243 | 0 | 0 | 0 | 100 | 0 | 0 | 0 |
| BRACE2005251 | 0 | 0 | 0 | 52.513 | 0 | 0 | 0 |
| BRACE2005253 | 0 | 0 | 0 | 39.025 | 0 | 0 | 0 |
| BRACE2005290 | 0 | 0 | 0 | 18.059 | 0 | 25.637 | 0 |
| BRACE2005298 | 0 | 0 | 0 | 21.618 | 0 | 0 | 0 |
| BRACE2005328 | 0 | 0 | 0 | 100 | 0 | 0 | 0 |
| BRACE2005329 | 0 | 0 | 0 | 100 | 0 | 0 | 0 |
| BRACE2005337 | 0 | 0 | 0 | 100 | 0 | 0 | 0 |
| BRACE2005348 | 0 | 0 | 0 | 100 | 0 | 0 | 0 |
| BRACE2005363 | 0 | 0 | 0 | 10.086 | 0 | 0 | 0 |
| BRACE2005395 | 0 | 0 | 0 | 20.076 | 0 | 0 | 0 |
| BRACE2005408 | 0 | 0 | 0 | 6.751 | 0 | 0 | 0 |
| BRACE2005433 | 0 | 0 | 0 | 8.197 | 0 | 0 | 0 |
| BRACE2005434 | 0 | 0 | 0 | 1.468 | 2.491 | 0 | 0 |
| BRACE2005448 | 0 | 0 | 0 | 9.653 | 0 | 0 | 0 |
| BRACE2005450 | 0 | 0 | 0 | 100 | 0 | 0 | 0 |
| BRACE2005453 | 0 | 0 | 0 | 9.182 | 0 | 0 | 0 |
| BRACE2005457 | 0 | 0 | 0 | 41.512 | 0 | 0 | 0 |
| BRACE2005460 | 0 | 0 | 0 | 100 | 0 | 0 | 0 |
| BRACE2005489 | 0 | 0 | 0 | 100 | 0 | 0 | 0 |
| BRACE2005518 | 0 | 0 | 0 | 100 | 0 | 0 | 0 |
| BRACE2005556 | 0 | 0 | 0 | 100 | 0 | 0 | 0 |
| BRACE2005562 | 0 | 0 | 0 | 100 | 0 | 0 | 0 |
| BRACE2005624 | 0 | 0 | 0 | 100 | 0 | 0 | 0 |
| BRACE2005642 | 0 | 0 | 0 | 25.930 | 0 | 7.362 | 0 |
| BRACE2005661 | 0 | 0 | 0 | 100 | 0 | 0 | 0 |
| BRACE2005667 | 0 | 0 | 0 | 100 | 0 | 0 | 0 |
| BRACE2005681 | 0 | 0 | 0 | 14.879 | 0 | 13.076 | 0 |
| BRACE2005719 | 0 | 0 | 0 | 6.324 | 32.194 | 5.985 | 0 |
| BRACE2005731 | 0 | 0 | 0 | 41.329 | 0 | 58.671 | 0 |
| BRACE2005742 | 0 | 0 | 0 | 100 | 0 | 0 | 0 |
| BRACE2005762 | 0 | 0 | 0 | 100 | 0 | 0 | 0 |
| BRACE2005773 | 0 | 0 | 0 | 100 | 0 | 0 | 0 |
| BRACE2005787 | 0 | 0 | 0 | 100 | 0 | 0 | 0 |
| BRACE2005795 | 0 | 0 | 0 | 100 | 0 | 0 | 0 |
| BRACE2005800 | 0 | 0 | 0 | 100 | 0 | 0 | 0 |
| BRACE2005858 | 0 | 0 | 0 | 14.668 | 0 | 0 | 0 |
| BRACE2005869 | 0 | 0 | 0 | 100 | 0 | 0 | 0 |
| BRACE2005875 | 0 | 0 | 0 | 3.056 | 0 | 0 | 0 |
| BRACE2005881 | 0 | 0 | 0 | 100 | 0 | 0 | 0 |
| BRACE2005898 | 0 | 0 | 0 | 100 | 0 | 0 | 0 |
| BRACE2005904 | 0 | 0 | 0 | 100 | 0 | 0 | 0 |
| BRACE2005906 | 0 | 0 | 0 | 100 | 0 | 0 | 0 |
| BRACE2005907 | 0 | 0 | 0 | 100 | 0 | 0 | 0 |
| BRACE2005911 | 0 | 0 | 0 | 100 | 0 | 0 | 0 |
| BRACE2005937 | 0 | 0 | 0 | 8.574 | 0 | 0 | 0 |
| BRACE2005949 | 0 | 0 | 0 | 100 | 0 | 0 | 0 |
| BRACE2005981 | 0 | 0 | 0 | 100 | 0 | 0 | 0 |
| BRACE2005991 | 0 | 0 | 0 | 100 | 0 | 0 | 0 |
| BRACE2006055 | 0 | 0 | 0 | 4.259 | 0 | 2.016 | 0 |
| BRACE2006072 | 0 | 0 | 0 | 13.990 | 0 | 0 | 0 |
| BRACE2006075 | 0 | 0 | 0 | 11.517 | 0 | 0 | 0 |
| BRACE2006084 | 0 | 0 | 0 | 2.179 | 0 | 3.093 | 0 |
| BRACE2006089 | 0 | 0 | 0 | 54.983 | 0 | 0 | 0 |
| BRACE2006102 | 0 | 0 | 0 | 29.854 | 0 | 0 | 0 |
| BRACE2006105 | 0 | 0 | 0 | 15.058 | 0 | 0 | 0 |
| BRACE2006122 | 0 | 0 | 0 | 100 | 0 | 0 | 0 |
| BRACE2006137 | 0 | 0 | 0 | 41.329 | 0 | 58.671 | 0 |
| BRACE2006143 | 0 | 0 | 0 | 100 | 0 | 0 | 0 |
| BRACE2006162 | 0 | 0 | 0 | 1.098 | 0 | 0 | 0 |
| BRACE2006167 | 0 | 0 | 0 | 10.473 | 15.234 | 14.868 | 0 |
| BRACE2006174 | 0 | 0 | 0 | 100 | 0 | 0 | 0 |
| BRACE2006240 | 0 | 0 | 0 | 100 | 0 | 0 | 0 |
| BRACE2006245 | 0 | 0 | 0 | 100 | 0 | 0 | 0 |
| BRACE2006249 | 0 | 0 | 0 | 45.981 | 0 | 0 | 0 |
| BRACE2006258 | 0 | 0 | 0 | 1.744 | 2.960 | 0 | 0 |
| BRACE2006264 | 0 | 0 | 0 | 5.990 | 0 | 0 | 0 |
| BRACE2006274 | 0 | 0 | 0 | 41.329 | 0 | 58.671 | 0 |
| BRACE2006281 | 0 | 0 | 0 | 2.145 | 0 | 0 | 0 |
| BRACE2006298 | 0 | 0 | 0 | 100 | 0 | 0 | 0 |
| BRACE2006319 | 0 | 0 | 0 | 4.977 | 0 | 3.532 | 0 |
| BRACE2006344 | 0 | 0 | 0 | 2.537 | 0 | 0 | 0 |
| BRACE2006354 | 0 | 0 | 0 | 4.417 | 22.485 | 0 | 0 |
| BRACE2006363 | 0 | 0 | 0 | 26.312 | 0 | 9.338 | 0 |
| BRACE2006378 | 0 | 0 | 0 | 38.787 | 0 | 0 | 0 |
| BRACE2006380 | 0 | 0 | 0 | 58.183 | 0 | 0 | 0 |
| BRACE2006393 | 0 | 0 | 0 | 100 | 0 | 0 | 0 |
| BRACE2006397 | 0 | 4.144 | 0 | 0.838 | 5.333 | 0.297 | 0 |
| BRACE2006413 | 0 | 0 | 0 | 100 | 0 | 0 | 0 |
| BRACE2006417 | 0 | 0 | 0 | 100 | 0 | 0 | 0 |
| BRACE2006453 | 0 | 0 | 0 | 100 | 0 | 0 | 0 |
| BRACE2006459 | 0 | 0 | 0 | 28.977 | 0 | 0 | 0 |
| BRACE2006488 | 0 | 0 | 0 | 100 | 0 | 0 | 0 |
| BRACE2006514 | 0 | 0 | 0 | 3.700 | 0 | 3.501 | 0 |
| BRACE2006534 | 0 | 0 | 0 | 100 | 0 | 0 | 0 |
| BRACE2006537 | 0 | 0 | 0 | 1.528 | 0 | 2.169 | 0 |
| BRACE2006540 | 0 | 0 | 0 | 100 | 0 | 0 | 0 |
| BRACE2006547 | 0 | 0 | 0 | 10.619 | 0 | 0 | 0 |
| BRACE2006564 | 0 | 0 | 0 | 29.854 | 0 | 0 | 0 |
| BRACE2006587 | 0 | 0 | 0 | 100 | 0 | 0 | 0 |
| BRACE2006598 | 0 | 0 | 0 | 20.531 | 0 | 29.147 | 0 |
| BRACE2006636 | 0 | 0 | 0 | 9.731 | 0 | 0 | 0 |
| BRACE2006685 | 0 | 0 | 0 | 100 | 0 | 0 | 0 |
| BRACE2006703 | 0 | 0 | 0 | 100 | 0 | 0 | 0 |
| BRACE2006743 | 0 | 0 | 0 | 15.779 | 0 | 4.480 | 0 |
| BRACE2006753 | 0 | 0 | 0 | 4.364 | 0 | 0 | 0 |
| BRACE2006783 | 0 | 0 | 0 | 100 | 0 | 0 | 0 |
| BRACE2006821 | 0 | 0 | 0 | 1.311 | 20.016 | 0 | 0 |
| BRACE2006833 | 0 | 0 | 0 | 53.329 | 0 | 0 | 0 |
| BRACE2006859 | 0 | 0 | 0 | 7.211 | 0 | 0 | 0 |
| BRACE2006871 | 0 | 0 | 0 | 0.710 | 0 | 3.022 | 0 |
| BRACE2006900 | 0 | 0 | 0 | 2.191 | 0 | 0 | 0 |
| BRACE2006909 | 0 | 0 | 0 | 100 | 0 | 0 | 0 |
| BRACE2006911 | 0 | 0 | 0 | 13.180 | 0 | 18.711 | 0 |
| BRACE2006924 | 0 | 0 | 0 | 100 | 0 | 0 | 0 |
| BRACE2006935 | 0 | 0 | 0 | 16.418 | 83.582 | 0 | 0 |
| BRACE2006937 | 0 | 0 | 0 | 4.750 | 4.836 | 0 | 0 |
| BRACE2006944 | 0 | 0 | 0 | 8.278 | 0 | 0 | 0 |
| BRACE2006951 | 0 | 0 | 0 | 100 | 0 | 0 | 0 |
| BRACE2006982 | 0 | 0 | 0 | 3.683 | 51.560 | 0 | 0 |
| BRACE2006986 | 0 | 0 | 0 | 100 | 0 | 0 | 0 |
| BRACE2007013 | 0 | 0 | 0 | 21.832 | 0 | 20.662 | 0 |
| BRACE2007064 | 0 | 11.405 | 0 | 3.460 | 0 | 3.274 | 0 |
| BRACE2007068 | 0 | 0 | 0 | 35.550 | 0 | 0 | 0 |
| BRACE2007087 | 0 | 0 | 0 | 100 | 0 | 0 | 0 |
| BRACE2007089 | 0 | 0 | 0 | 0.601 | 0 | 0.853 | 0 |
| BRACE2007109 | 0 | 0 | 0 | 25.067 | 3.449 | 5.771 | 0 |
| BRACE2007138 | 0 | 0 | 0 | 100 | 0 | 0 | 0 |
| BRACE2007174 | 0 | 0 | 0 | 11.088 | 0 | 0 | 0 |
| BRACE2007191 | 0 | 0 | 0 | 2.438 | 0 | 0.692 | 0 |
| BRACE2007197 | 0 | 0 | 0 | 100 | 0 | 0 | 0 |
| BRACE2007201 | 0 | 0 | 0 | 41.027 | 0 | 0 | 0 |
| BRACE2007203 | 0 | 0 | 0 | 100 | 0 | 0 | 0 |
| BRACE2007232 | 0 | 0 | 0 | 100 | 0 | 0 | 0 |
| BRACE2007254 | 0 | 0 | 0 | 5.283 | 0 | 0 | 0 |
| BRACE2007262 | 0 | 0 | 0 | 100 | 0 | 0 | 0 |
| BRACE2007274 | 0 | 0 | 0 | 100 | 0 | 0 | 0 |
| BRACE2007281 | 0 | 0 | 0 | 100 | 0 | 0 | 0 |
| BRACE2007295 | 0 | 0 | 0 | 26.697 | 0 | 0 | 0 |
| BRACE2007358 | 0 | 0 | 0 | 100 | 0 | 0 | 0 |
| BRACE2007396 | 0 | 0 | 0 | 100 | 0 | 0 | 0 |
| BRACE2007398 | 0 | 0 | 0 | 100 | 0 | 0 | 0 |
| BRACE2007401 | 0 | 0 | 0 | 7.783 | 6.604 | 7.366 | 0 |
| BRACE2007411 | 0 | 0 | 0 | 100 | 0 | 0 | 0 |
| BRACE2007447 | 0 | 0 | 0 | 100 | 0 | 0 | 0 |
| BRACE2007477 | 0 | 0 | 0 | 100 | 0 | 0 | 0 |
| BRACE2007502 | 0 | 0 | 0 | 100 | 0 | 0 | 0 |
| BRACE2007503 | 0 | 0 | 0 | 100 | 0 | 0 | 0 |
| BRACE2007518 | 0 | 0 | 0 | 12.887 | 0 | 9.147 | 0 |
| BRACE2007522 | 0 | 0 | 0 | 41.027 | 0 | 0 | 0 |
| BRACE2007527 | 0 | 0 | 0 | 100 | 0 | 0 | 0 |
| BRACE2007538 | 0 | 0 | 0 | 100 | 0 | 0 | 0 |
| BRACE2007563 | 0 | 0 | 0 | 100 | 0 | 0 | 0 |
| BRACE2007567 | 0 | 0 | 0 | 1.970 | 0 | 2.796 | 0 |
| BRACE2007577 | 0 | 0 | 0 | 2.085 | 0 | 0 | 0 |
| BRACE2007621 | 0 | 0 | 0 | 100 | 0 | 0 | 0 |
| BRACE2007640 | 0 | 0 | 0 | 100 | 0 | 0 | 0 |
| BRACE2007641 | 0 | 0 | 0 | 10.014 | 5.664 | 3.159 | 0 |
| BRACE2007646 | 0 | 0 | 0 | 41.329 | 0 | 58.671 | 0 |
| BRACE2007663 | 0 | 0 | 0 | 67.607 | 0 | 0 | 0 |
| BRACE2007671 | 0 | 0 | 0 | 1.097 | 0 | 0 | 0 |
| BRACE2007682 | 0 | 0 | 0 | 100 | 0 | 0 | 0 |
| BRACE2007685 | 0 | 0 | 0 | 100 | 0 | 0 | 0 |
| BRACE2007708 | 0 | 0 | 0 | 3.017 | 0 | 2.141 | 0 |
| BRACE2007727 | 0 | 0 | 0 | 100 | 0 | 0 | 0 |
| BRACE2007749 | 0 | 0 | 0 | 100 | 0 | 0 | 0 |
| BRACE2007760 | 0 | 0 | 0 | 83.719 | 0 | 0 | 0 |
| BRACE2007761 | 0 | 0 | 0 | 100 | 0 | 0 | 0 |
| BRACE2007764 | 0 | 0 | 0 | 41.329 | 0 | 58.671 | 0 |
| BRACE2007767 | 0 | 0 | 0 | 2.210 | 0 | 0 | 0 |
| BRACE2007768 | 0 | 0 | 0 | 6.791 | 0 | 0 | 0 |
| BRACE2007769 | 0 | 0 | 0 | 100 | 0 | 0 | 0 |
| BRACE2007784 | 0 | 0 | 0 | 100 | 0 | 0 | 0 |
| BRACE2007786 | 0 | 0 | 0 | 100 | 0 | 0 | 0 |
| BRACE2007798 | 0 | 0 | 0 | 2.937 | 7.475 | 4.169 | 0 |
| BRACE2007799 | 0 | 0 | 0 | 100 | 0 | 0 | 0 |
| BRACE2007801 | 0 | 0 | 0 | 52.057 | 0 | 0 | 0 |
| BRACE2007832 | 0 | 0 | 0 | 17.815 | 0 | 25.291 | 0 |
| BRACE2007836 | 0 | 0 | 0 | 100 | 0 | 0 | 0 |
| BRACE2007868 | 0 | 0 | 0 | 100 | 0 | 0 | 0 |
| BRACE2007882 | 0 | 0 | 0 | 32.721 | 0 | 6.636 | 0 |
| BRACE2007888 | 0 | 0 | 0 | 100 | 0 | 0 | 0 |
| BRACE2007902 | 0 | 0 | 0 | 22.750 | 0 | 0 | 0 |
| BRACE2007920 | 0 | 0 | 0 | 100 | 0 | 0 | 0 |
| BRACE2007937 | 0 | 0 | 0 | 5.408 | 0 | 7.677 | 0 |
| BRACE2007944 | 0 | 0 | 0 | 100 | 0 | 0 | 0 |
| BRACE2007952 | 0 | 0 | 0 | 8.604 | 8.761 | 9.772 | 0 |
| BRACE2008008 | 0 | 0 | 0 | 100 | 0 | 0 | 0 |
| BRACE2008015 | 0 | 0 | 0 | 100 | 0 | 0 | 0 |
| BRACE2008021 | 0 | 0 | 0 | 100 | 0 | 0 | 0 |
| BRACE2008097 | 0 | 0 | 0 | 3.408 | 0 | 0 | 0 |
| BRACE2008102 | 0 | 0 | 0 | 16.858 | 0 | 23.932 | 0 |
| BRACE2008114 | 0 | 0 | 0 | 100 | 0 | 0 | 0 |
| BRACE2008144 | 0 | 0 | 0 | 100 | 0 | 0 | 0 |
| BRACE2008172 | 0 | 0 | 0 | 100 | 0 | 0 | 0 |
| BRACE2008216 | 0 | 0 | 0 | 9.397 | 0 | 0 | 0 |
| BRACE2008218 | 0 | 0 | 0 | 100 | 0 | 0 | 0 |
| BRACE2008251 | 0 | 0 | 0 | 19.231 | 0 | 0 | 0 |
| BRACE2008276 | 0 | 0 | 0 | 100 | 0 | 0 | 0 |
| BRACE2008295 | 0 | 0 | 0 | 100 | 0 | 0 | 0 |
| BRACE2008308 | 0 | 0 | 0 | 100 | 0 | 0 | 0 |
| BRACE2008336 | 0 | 0 | 0 | 100 | 0 | 0 | 0 |
| BRACE2008358 | 0 | 0 | 0 | 3.217 | 0 | 6.850 | 0 |
| BRACE2008361 | 0 | 0 | 0 | 100 | 0 | 0 | 0 |
| BRACE2008380 | 0 | 0 | 0 | 1.387 | 14.119 | 5.906 | 0 |
| BRACE2008385 | 0 | 0 | 0 | 2.965 | 0 | 0 | 0 |
| BRACE2008399 | 0 | 0 | 0 | 3.928 | 0 | 0 | 0 |
| BRACE2008401 | 0 | 0 | 0 | 100 | 0 | 0 | 0 |
| BRACE2008402 | 0 | 0 | 0 | 5.953 | 0 | 0 | 0 |
| BRACE2008410 | 0 | 0 | 0 | 100 | 0 | 0 | 0 |
| BRACE2008420 | 0 | 0 | 0 | 100 | 0 | 0 | 0 |
| BRACE2008443 | 0 | 0 | 0 | 100 | 0 | 0 | 0 |
| BRACE2008473 | 0 | 0 | 0 | 100 | 0 | 0 | 0 |
| BRACE2008480 | 0 | 0 | 0 | 100 | 0 | 0 | 0 |
| BRACE2008481 | 0 | 0 | 0 | 3.963 | 10.087 | 2.813 | 0 |
| BRACE2008488 | 0 | 0 | 0 | 100 | 0 | 0 | 0 |
| BRACE2008495 | 0 | 0 | 0 | 16.214 | 0 | 0 | 0 |
| BRACE2008500 | 0 | 0 | 0 | 100 | 0 | 0 | 0 |
| BRACE2008553 | 0 | 0 | 0 | 2.952 | 0 | 4.191 | 0 |
| BRACE2008594 | 0 | 0 | 0 | 100 | 0 | 0 | 0 |
| BRACE2008604 | 0 | 0 | 0 | 100 | 0 | 0 | 0 |
| BRACE2008615 | 0 | 0 | 0 | 100 | 0 | 0 | 0 |
| BRACE2008622 | 0 | 0 | 0 | 100 | 0 | 0 | 0 |
| BRACE2008629 | 0 | 0 | 0 | 100 | 0 | 0 | 0 |
| BRACE2008653 | 0 | 0 | 4.067 | 8.610 | 0 | 2.126 | 0 |
| BRACE2008655 | 0 | 0 | 0 | 100 | 0 | 0 | 0 |
| BRACE2008656 | 0 | 0 | 0 | 100 | 0 | 0 | 0 |
| BRACE2008706 | 0 | 0 | 0 | 11.660 | 0 | 0 | 0 |
| BRACE2008708 | 0 | 0 | 0 | 49.602 | 0 | 0 | 0 |
| BRACE2008710 | 0 | 0 | 0 | 100 | 0 | 0 | 0 |
| BRACE2008754 | 0 | 0 | 0 | 100 | 0 | 0 | 0 |
| BRACE2008797 | 0 | 0 | 0 | 44.047 | 0 | 25.012 | 0 |
| BRACE2008847 | 0 | 0 | 0 | 100 | 0 | 0 | 0 |
| BRACE2008857 | 0 | 0 | 0 | 41.512 | 0 | 0 | 0 |
| BRACE2008861 | 0 | 0 | 0 | 100 | 0 | 0 | 0 |
| BRACE2008867 | 0 | 0 | 0 | 100 | 0 | 0 | 0 |
| BRACE2008881 | 0 | 0 | 0 | 19.678 | 0 | 0 | 0 |
| BRACE2008883 | 0 | 0 | 0 | 100 | 0 | 0 | 0 |
| BRACE2008917 | 0 | 0 | 0 | 100 | 0 | 0 | 0 |
| BRACE2008919 | 0 | 0 | 0 | 100 | 0 | 0 | 0 |
| BRACE2008941 | 0 | 0 | 0 | 14.187 | 0 | 0 | 0 |
| BRACE2008948 | 0 | 0 | 0 | 100 | 0 | 0 | 0 |
| BRACE2008960 | 0 | 0 | 0 | 100 | 0 | 0 | 0 |
| BRACE2008968 | 18.226 | 0 | 0 | 2.476 | 0 | 0.586 | 0 |
| BRACE2008999 | 0 | 0 | 0 | 24.977 | 25.431 | 0 | 0 |
| BRACE2009014 | 0 | 0 | 0 | 100 | 0 | 0 | 0 |
| BRACE2009016 | 0 | 0 | 0 | 100 | 0 | 0 | 0 |
| BRACE2009031 | 0 | 0 | 0 | 100 | 0 | 0 | 0 |
| BRACE2009037 | 0 | 0 | 2.252 | 0.829 | 0 | 0.883 | 0 |
| BRACE2009044 | 0 | 0 | 0 | 7.121 | 0 | 0 | 0 |
| BRACE2009045 | 0 | 0 | 0 | 8.547 | 0 | 6.067 | 0 |
| BRACE2009053 | 0 | 0 | 0 | 100 | 0 | 0 | 0 |
| BRACE2009122 | 0 | 0 | 0 | 100 | 0 | 0 | 0 |
| BRACE2009131 | 0 | 0 | 0 | 100 | 0 | 0 | 0 |
| BRACE2009185 | 0 | 0 | 0 | 100 | 0 | 0 | 0 |
| BRACE2009188 | 0 | 0 | 0 | 100 | 0 | 0 | 0 |
| BRACE2009212 | 0 | 0 | 0 | 100 | 0 | 0 | 0 |
| BRACE2009221 | 0 | 0 | 0 | 100 | 0 | 0 | 0 |
| BRACE2009235 | 0 | 0 | 0 | 100 | 0 | 0 | 0 |
| BRACE2009238 | 0 | 0 | 0 | 100 | 0 | 0 | 0 |
| BRACE2009255 | 0 | 0 | 0 | 16.858 | 0 | 23.932 | 0 |
| BRACE2009270 | 0 | 0 | 0 | 100 | 0 | 0 | 0 |
| BRACE2009272 | 0 | 0 | 0 | 100 | 0 | 0 | 0 |
| BRACE2009274 | 0 | 0 | 0 | 100 | 0 | 0 | 0 |
| BRACE2009275 | 0 | 0 | 0 | 100 | 0 | 0 | 0 |
| BRACE2009291 | 0 | 0 | 0 | 1.878 | 0 | 0 | 0 |
| BRACE2009293 | 0 | 0 | 0 | 100 | 0 | 0 | 0 |
| BRACE2009307 | 0 | 0 | 0 | 21.024 | 0 | 0 | 0 |
| BRACE2009311 | 0 | 0 | 0 | 41.329 | 0 | 58.671 | 0 |
| BRACE2009318 | 0 | 0 | 0 | 100 | 0 | 0 | 0 |
| BRACE2009323 | 0 | 0 | 0 | 100 | 0 | 0 | 0 |
| BRACE2009366 | 0 | 0 | 0 | 2.564 | 0 | 0 | 0 |
| BRACE2009421 | 0 | 0 | 0 | 100 | 0 | 0 | 0 |
| BRACE2009428 | 0 | 0 | 0 | 100 | 0 | 0 | 0 |
| BRACE2009434 | 0 | 0 | 0 | 100 | 0 | 0 | 0 |
| BRACE2009437 | 0 | 0 | 0 | 100 | 0 | 0 | 0 |
| BRACE2009483 | 0 | 61.671 | 0 | 12.473 | 0 | 0 | 0 |
| BRACE2009517 | 0 | 0 | 0 | 3.850 | 0 | 2.733 | 0 |
| BRACE2009533 | 0 | 0 | 0 | 100 | 0 | 0 | 0 |
| BRACE2009558 | 0 | 0 | 0 | 4.082 | 0 | 2.897 | 0 |
| BRACE2009566 | 0 | 0 | 0 | 13.461 | 0 | 0 | 0 |
| BRACE2009591 | 0 | 0 | 0 | 100 | 0 | 0 | 0 |
| BRACE2009620 | 0 | 0 | 0 | 13.619 | 0 | 0 | 0 |
| BRACE2009654 | 0 | 0 | 0 | 100 | 0 | 0 | 0 |
| BRACE2009721 | 0 | 0 | 0 | 17.003 | 0 | 0 | 0 |
| BRACE2009732 | 0 | 0 | 0 | 100 | 0 | 0 | 0 |
| BRACE2009754 | 0 | 0 | 0 | 100 | 0 | 0 | 0 |
| BRACE2009828 | 0 | 0 | 0 | 26.047 | 0 | 73.953 | 0 |
| BRACE2009886 | 0 | 0 | 0 | 100 | 0 | 0 | 0 |
| BRACE2009907 | 0 | 0 | 0 | 100 | 0 | 0 | 0 |
| BRACE2009957 | 0 | 0 | 0 | 27.755 | 0 | 0 | 0 |
| BRACE2010170 | 0 | 0 | 0 | 100 | 0 | 0 | 0 |
| BRACE2010171 | 0 | 0 | 0 | 100 | 0 | 0 | 0 |
| BRACE2010203 | 0 | 0 | 0 | 100 | 0 | 0 | 0 |
| BRACE2010208 | 0 | 0 | 0 | 100 | 0 | 0 | 0 |
| BRACE2010336 | 0 | 0 | 0 | 9.658 | 0 | 0 | 0 |
| BRACE2010348 | 0 | 0 | 0 | 100 | 0 | 0 | 0 |
| BRACE2010440 | 0 | 0 | 0 | 100 | 0 | 0 | 0 |
| BRACE2010444 | 0 | 0 | 0 | 100 | 0 | 0 | 0 |
| BRACE2010477 | 0 | 0 | 0 | 100 | 0 | 0 | 0 |
| BRACE2010489 | 0 | 0 | 0 | 15.026 | 0 | 10.665 | 0 |
| BRACE2010535 | 0 | 0 | 0 | 100 | 0 | 0 | 0 |
| BRACE2010550 | 0 | 0 | 0 | 100 | 0 | 0 | 0 |
| BRACE2010598 | 0 | 0 | 0 | 100 | 0 | 0 | 0 |
| BRACE2010632 | 0 | 0 | 0 | 100 | 0 | 0 | 0 |
| BRACE2010642 | 0 | 0 | 0 | 10.913 | 0 | 0 | 0 |
| BRACE2010651 | 0 | 0 | 0 | 13.116 | 0 | 0 | 0 |
| BRACE2010669 | 0 | 0 | 0 | 9.049 | 0 | 0 | 0 |
| BRACE2010684 | 0 | 0 | 0 | 38.787 | 0 | 0 | 0 |
| BRACE2010753 | 0 | 0 | 0 | 13.239 | 0 | 0 | 0 |
| BRACE2010813 | 0 | 0 | 0 | 100 | 0 | 0 | 0 |
| BRACE2010828 | 0 | 0 | 0 | 27.755 | 0 | 0 | 0 |
| BRACE2010837 | 0 | 0 | 0 | 100 | 0 | 0 | 0 |
| BRACE2010888 | 0 | 0 | 0 | 100 | 0 | 0 | 0 |
| BRACE2010937 | 0 | 0 | 0 | 100 | 0 | 0 | 0 |
| BRACE2010983 | 0 | 0 | 0 | 100 | 0 | 0 | 0 |
| BRACE2011007 | 0 | 0 | 0 | 100 | 0 | 0 | 0 |
| BRACE2011025 | 0 | 0 | 0 | 100 | 0 | 0 | 0 |
| BRACE2011183 | 0 | 0 | 0 | 22.812 | 0 | 6.477 | 0 |
| BRACE2011199 | 0 | 0 | 0 | 100 | 0 | 0 | 0 |
| BRACE2011265 | 0 | 0 | 0 | 2.633 | 0 | 0 | 0 |
| BRACE2011389 | 0 | 0 | 0 | 100 | 0 | 0 | 0 |
| BRACE2011478 | 0 | 0 | 0 | 100 | 0 | 0 | 0 |
| BRACE2011545 | 0 | 0 | 0 | 100 | 0 | 0 | 0 |
| BRACE2011592 | 0 | 0 | 0 | 100 | 0 | 0 | 0 |
| BRACE2011611 | 0 | 0 | 0 | 100 | 0 | 0 | 0 |
| BRACE2011646 | 0 | 0 | 0 | 100 | 0 | 0 | 0 |
| BRACE2011677 | 0 | 0 | 0 | 100 | 0 | 0 | 0 |
| BRACE2011703 | 0 | 0 | 0 | 28.977 | 0 | 0 | 0 |
| BRACE2011747 | 0 | 0 | 0 | 100 | 0 | 0 | 0 |
| BRACE2011838 | 0 | 0 | 0 | 100 | 0 | 0 | 0 |
| BRACE2011892 | 0 | 0 | 0 | 100 | 0 | 0 | 0 |
| BRACE2011904 | 0 | 0 | 0 | 100 | 0 | 0 | 0 |
| BRACE2011947 | 0 | 0 | 0 | 100 | 0 | 0 | 0 |
| BRACE2012061 | 0 | 0 | 0 | 11.958 | 0 | 0 | 0 |
| BRACE2012062 | 0 | 0 | 0 | 100 | 0 | 0 | 0 |
| BRACE2012185 | 0 | 0 | 0 | 100 | 0 | 0 | 0 |
| BRACE2012222 | 0 | 0 | 0 | 60.579 | 0 | 0 | 0 |
| BRACE2012232 | 0 | 0 | 0 | 41.329 | 0 | 58.671 | 0 |
| BRACE2012291 | 0 | 0 | 0 | 27.755 | 0 | 0 | 0 |
| BRACE2012317 | 0 | 0 | 0 | 13.150 | 0 | 13.828 | 0 |
| BRACE2012510 | 0 | 0 | 0 | 100 | 0 | 0 | 0 |
| BRACE2012528 | 0 | 0 | 0 | 15.820 | 1.714 | 10.512 | 0 |
| BRACE2012625 | 0 | 0 | 0 | 100 | 0 | 0 | 0 |
| BRACE2012653 | 0 | 0 | 0 | 41.329 | 0 | 58.671 | 0 |
| BRACE2012814 | 0 | 0 | 0 | 8.707 | 0 | 1.545 | 0 |
| BRACE2012833 | 0 | 0 | 0 | 100 | 0 | 0 | 0 |
| BRACE2012838 | 0 | 0 | 0 | 100 | 0 | 0 | 0 |
| BRACE2012936 | 0 | 0 | 0 | 100 | 0 | 0 | 0 |
| BRACE2012947 | 0 | 0 | 0 | 100 | 0 | 0 | 0 |
| BRACE2013009 | 0 | 0 | 0 | 100 | 0 | 0 | 0 |
| BRACE2013010 | 0 | 0 | 0 | 100 | 0 | 0 | 0 |
| BRACE2013126 | 0 | 0 | 0 | 100 | 0 | 0 | 0 |
| BRACE2013132 | 0 | 0 | 0 | 100 | 0 | 0 | 0 |
| BRACE2013149 | 0 | 0 | 0 | 100 | 0 | 0 | 0 |
| BRACE2013369 | 0 | 0 | 0 | 22.162 | 0 | 0 | 0 |
| BRACE2013806 | 0 | 0 | 0 | 100 | 0 | 0 | 0 |
| BRACE2013855 | 0 | 0 | 0 | 11.386 | 0 | 0 | 0 |
| BRACE2013911 | 0 | 0 | 0 | 5.335 | 0 | 7.574 | 0 |
| BRACE2014108 | 0 | 0 | 0 | 100 | 0 | 0 | 0 |
| BRACE2014161 | 0 | 0 | 0 | 100 | 0 | 0 | 0 |
| BRACE2014232 | 0 | 0 | 0 | 100 | 0 | 0 | 0 |
| BRACE2014257 | 0 | 0 | 0 | 44.008 | 0 | 0 | 0 |
| BRACE2014268 | 0 | 0 | 0 | 100 | 0 | 0 | 0 |
| BRACE2014306 | 0 | 0 | 0 | 6.909 | 0 | 0 | 0 |
| BRACE2014475 | 0 | 0 | 0 | 100 | 0 | 0 | 0 |
| BRACE2014657 | 0 | 0 | 0 | 76.765 | 0 | 7.784 | 0 |
| BRACE2014746 | 0 | 0 | 0 | 9.906 | 25.214 | 7.031 | 0 |
| BRACE2014780 | 0 | 0 | 0 | 100 | 0 | 0 | 0 |
| BRACE2014818 | 0 | 0 | 0 | 28.977 | 0 | 0 | 0 |
| BRACE2014821 | 0 | 0 | 0 | 100 | 0 | 0 | 0 |
| BRACE2014824 | 0 | 0 | 0 | 100 | 0 | 0 | 0 |
| BRACE2014850 | 0 | 52.642 | 0 | 15.970 | 0 | 0 | 0 |
| BRACE2014854 | 0 | 0 | 0 | 100 | 0 | 0 | 0 |
| BRACE2015031 | 0 | 0 | 0 | 100 | 0 | 0 | 0 |
| BRACE2015058 | 0 | 0 | 0 | 6.224 | 0 | 8.836 | 0 |
| BRACE2015132 | 0 | 0 | 0 | 11.694 | 0 | 0 | 0 |
| BRACE2015262 | 0 | 0 | 0 | 100 | 0 | 0 | 0 |
| BRACE2015270 | 0 | 0 | 0 | 100 | 0 | 0 | 0 |
| BRACE2015287 | 0 | 0 | 0 | 1.630 | 0 | 0 | 0 |
| BRACE2015314 | 0 | 0 | 0 | 100 | 0 | 0 | 0 |
| BRACE2015352 | 0 | 0 | 0 | 100 | 0 | 0 | 0 |
| BRACE2015368 | 0 | 0 | 0 | 41.512 | 0 | 0 | 0 |
| BRACE2015412 | 0 | 0 | 0 | 17.282 | 0 | 0 | 0 |
| BRACE2015436 | 0 | 0 | 0 | 100 | 0 | 0 | 0 |
| BRACE2015741 | 0 | 0 | 0 | 100 | 0 | 0 | 0 |
| BRACE2015756 | 0 | 0 | 0 | 100 | 0 | 0 | 0 |
| BRACE2015969 | 0 | 0 | 0 | 28.977 | 0 | 0 | 0 |
| BRACE2015995 | 0 | 0 | 0 | 100 | 0 | 0 | 0 |
| BRACE2016058 | 0 | 0 | 0 | 100 | 0 | 0 | 0 |
| BRACE2016194 | 0 | 0 | 0 | 23.627 | 0 | 0 | 0 |
| BRACE2016315 | 0 | 0 | 0 | 100 | 0 | 0 | 0 |
| BRACE2016325 | 0 | 0 | 0 | 100 | 0 | 0 | 0 |
| BRACE2016335 | 0 | 0 | 0 | 9.897 | 0 | 0 | 0 |
| BRACE2016362 | 0 | 0 | 0 | 32.824 | 0 | 15.533 | 0 |
| BRACE2016366 | 0 | 0 | 0 | 12.062 | 30.702 | 0 | 0 |
| BRACE2016465 | 0 | 0 | 0 | 100 | 0 | 0 | 0 |
| BRACE2016516 | 0 | 0 | 0 | 100 | 0 | 0 | 0 |
| BRACE2016583 | 0 | 0 | 0 | 100 | 0 | 0 | 0 |
| BRACE2016708 | 0 | 0 | 0 | 100 | 0 | 0 | 0 |
| BRACE2016821 | 0 | 0 | 0 | 100 | 0 | 0 | 0 |
| BRACE2016896 | 0 | 0 | 0 | 100 | 0 | 0 | 0 |
| BRACE2016981 | 0 | 0 | 0 | 16.089 | 0 | 0 | 0 |
| BRACE2017108 | 0 | 0 | 0 | 100 | 0 | 0 | 0 |
| BRACE2017124 | 0 | 0 | 0 | 100 | 0 | 0 | 0 |
| BRACE2017298 | 0 | 0 | 0 | 100 | 0 | 0 | 0 |
| BRACE2017359 | 0 | 0 | 0 | 58.183 | 0 | 0 | 0 |
| BRACE2017397 | 0 | 0 | 0 | 3.348 | 0 | 2.376 | 0 |
| BRACE2017410 | 0 | 0 | 0 | 100 | 0 | 0 | 0 |
| BRACE2017438 | 0 | 0 | 0 | 58.486 | 0 | 41.514 | 0 |
| BRACE2017574 | 0 | 0 | 0 | 7.273 | 0 | 0 | 0 |
| BRACE2017580 | 0 | 0 | 0 | 100 | 0 | 0 | 0 |
| BRACE2017587 | 0 | 0 | 0 | 100 | 0 | 0 | 0 |
| BRACE2017720 | 0 | 0 | 0 | 100 | 0 | 0 | 0 |
| BRACE2017844 | 0 | 0 | 0 | 29.179 | 0 | 15.534 | 0 |
| BRACE2017872 | 0 | 0 | 0 | 100 | 0 | 0 | 0 |
| BRACE2017929 | 0 | 0 | 0 | 100 | 0 | 0 | 0 |
| BRACE2017934 | 0 | 0 | 0 | 13.120 | 0 | 0 | 0 |
| BRACE2017985 | 0 | 0 | 0 | 38.787 | 0 | 0 | 0 |
| BRACE2017992 | 0 | 0 | 0 | 100 | 0 | 0 | 0 |
| BRACE2018337 | 0 | 0 | 0 | 1.691 | 0 | 0 | 0 |
| BRACE2018443 | 0 | 0 | 0 | 45.981 | 0 | 0 | 0 |
| BRACE2018448 | 0 | 0 | 0 | 100 | 0 | 0 | 0 |
| BRACE2018568 | 0 | 0 | 0 | 100 | 0 | 0 | 0 |
| BRACE2018700 | 0 | 0 | 0 | 100 | 0 | 0 | 0 |
| BRACE2018727 | 0 | 0 | 0 | 100 | 0 | 0 | 0 |
| BRACE2018736 | 0 | 0 | 0 | 28.707 | 0 | 0 | 0 |
| BRACE2018759 | 0 | 0 | 0 | 100 | 0 | 0 | 0 |
| BRACE2018762 | 0 | 0 | 0 | 41.027 | 0 | 0 | 0 |
| BRACE2018782 | 0 | 0 | 0 | 100 | 0 | 0 | 0 |
| BRACE2018847 | 0 | 0 | 0 | 13.846 | 0 | 0 | 0 |
| BRACE2019004 | 0 | 0 | 0 | 39.025 | 0 | 0 | 0 |
| BRACE2019044 | 0 | 0 | 0 | 100 | 0 | 0 | 0 |
| BRACE2019147 | 0 | 0 | 0 | 1.520 | 0 | 0 | 0 |
| BRACE2019244 | 0 | 0 | 0 | 28.977 | 0 | 0 | 0 |
| BRACE2019258 | 0 | 0 | 0 | 100 | 0 | 0 | 0 |
| BRACE2019327 | 0 | 0 | 0 | 6.360 | 0 | 0 | 0 |
| BRACE2019348 | 0 | 0 | 0 | 74.228 | 0 | 0 | 0 |
| BRACE2019371 | 0 | 0 | 0 | 21.225 | 0 | 0 | 0 |
| BRACE2019467 | 0 | 0 | 0 | 100 | 0 | 0 | 0 |
| BRACE2019510 | 0 | 0 | 0 | 100 | 0 | 0 | 0 |
| BRACE2019519 | 0 | 0 | 0 | 13.211 | 0 | 0 | 0 |
| BRACE2019528 | 0 | 0 | 0 | 100 | 0 | 0 | 0 |
| BRACE2019618 | 0 | 0 | 0 | 100 | 0 | 0 | 0 |
| BRACE2019696 | 0 | 0 | 0 | 100 | 0 | 0 | 0 |
| BRACE2019703 | 0 | 0 | 0 | 100 | 0 | 0 | 0 |
| BRACE2019843 | 0 | 0 | 0 | 100 | 0 | 0 | 0 |
| BRACE2019865 | 0 | 0 | 0 | 100 | 0 | 0 | 0 |
| BRACE2019872 | 0 | 0 | 0 | 100 | 0 | 0 | 0 |
| BRACE2019877 | 0 | 0 | 0 | 8.622 | 0 | 6.120 | 0 |
| BRACE2020028 | 0 | 0 | 0 | 100 | 0 | 0 | 0 |
| BRACE2020077 | 0 | 0 | 0 | 100 | 0 | 0 | 0 |
| BRACE2020097 | 0 | 0 | 0 | 100 | 0 | 0 | 0 |
| BRACE2020157 | 0 | 0 | 0 | 100 | 0 | 0 | 0 |
| BRACE2020278 | 0 | 0 | 0 | 100 | 0 | 0 | 0 |
| BRACE2020358 | 0 | 0 | 0 | 100 | 0 | 0 | 0 |
| BRACE2020467 | 0 | 0 | 0 | 100 | 0 | 0 | 0 |
| BRACE2020584 | 0 | 0 | 0 | 37.308 | 0 | 0 | 0 |
| BRACE2020652 | 0 | 0 | 0 | 100 | 0 | 0 | 0 |
| BRACE2020709 | 0 | 0 | 0 | 100 | 0 | 0 | 0 |
| BRACE2020742 | 0 | 0 | 0 | 100 | 0 | 0 | 0 |
| BRACE2020756 | 0 | 0 | 0 | 100 | 0 | 0 | 0 |
| BRACE2020863 | 0 | 0 | 0 | 100 | 0 | 0 | 0 |
| BRACE2020982 | 0 | 0 | 0 | 100 | 0 | 0 | 0 |
| BRACE2021014 | 0 | 0 | 0 | 4.205 | 0 | 0 | 0 |
| BRACE2021143 | 0 | 0 | 0 | 11.233 | 0 | 0 | 0 |
| BRACE2021148 | 0 | 0 | 0 | 100 | 0 | 0 | 0 |
| BRACE2021245 | 0 | 0 | 0 | 100 | 0 | 0 | 0 |
| BRACE2021254 | 0 | 0 | 0 | 100 | 0 | 0 | 0 |
| BRACE2021264 | 0 | 0 | 0 | 100 | 0 | 0 | 0 |
| BRACE2021344 | 0 | 0 | 0 | 22.162 | 0 | 0 | 0 |
| BRACE2021433 | 0 | 0 | 0 | 100 | 0 | 0 | 0 |
| BRACE2021478 | 0 | 0 | 0 | 9.188 | 0 | 0 | 0 |
| BRACE2021541 | 0 | 0 | 0 | 11.299 | 0 | 32.080 | 0 |
| BRACE2021670 | 0 | 0 | 0 | 100 | 0 | 0 | 0 |
| BRACE2021695 | 0 | 0 | 0 | 100 | 0 | 0 | 0 |
| BRACE2021708 | 0 | 0 | 0 | 21.015 | 0 | 0 | 0 |
| BRACE2021721 | 0 | 0 | 0 | 100 | 0 | 0 | 0 |
| BRACE2021932 | 0 | 0 | 0 | 41.027 | 0 | 0 | 0 |
| BRACE2021936 | 0 | 0 | 0 | 100 | 0 | 0 | 0 |
| BRACE2022030 | 0 | 0 | 0 | 100 | 0 | 0 | 0 |
| BRACE2022158 | 0 | 0 | 0 | 100 | 0 | 0 | 0 |
| BRACE2022196 | 0 | 0 | 0 | 100 | 0 | 0 | 0 |
| BRACE2022270 | 0 | 0 | 0 | 100 | 0 | 0 | 0 |
| BRACE2022328 | 0 | 0 | 0 | 100 | 0 | 0 | 0 |
| BRACE2022333 | 0 | 0 | 0 | 12.602 | 0 | 0 | 0 |
| BRACE2022389 | 0 | 0 | 0 | 7.542 | 0 | 0 | 0 |
| BRACE2022394 | 0 | 0 | 0 | 100 | 0 | 0 | 0 |
| BRACE2022435 | 0 | 0 | 0 | 100 | 0 | 0 | 0 |
| BRACE2022448 | 0 | 0 | 0 | 100 | 0 | 0 | 0 |
| BRACE2022450 | 0 | 0 | 0 | 100 | 0 | 0 | 0 |
| BRACE2022549 | 0 | 0 | 0 | 36.360 | 0 | 0 | 0 |
| BRACE2022633 | 0 | 0 | 0 | 100 | 0 | 0 | 0 |
| BRACE2022638 | 23.363 | 0 | 0 | 4.761 | 0 | 0.751 | 0 |
| BRACE2022693 | 0 | 0 | 0 | 100 | 0 | 0 | 0 |
| BRACE2022848 | 0 | 0 | 0 | 100 | 0 | 0 | 0 |
| BRACE2022928 | 0 | 0 | 0 | 100 | 0 | 0 | 0 |
| BRACE2022990 | 0 | 0 | 0 | 41.329 | 0 | 58.671 | 0 |
| BRACE2023069 | 0 | 0 | 0 | 100 | 0 | 0 | 0 |
| BRACE2023070 | 0 | 0 | 0 | 100 | 0 | 0 | 0 |
| BRACE2023223 | 0 | 0 | 0 | 25.927 | 0 | 36.806 | 0 |
| BRACE2023268 | 0 | 0 | 0 | 100 | 0 | 0 | 0 |
| BRACE2023284 | 0 | 0 | 0 | 100 | 0 | 0 | 0 |
| BRACE2023404 | 0 | 0 | 0 | 100 | 0 | 0 | 0 |
| BRACE2023451 | 0 | 0 | 0 | 100 | 0 | 0 | 0 |
| BRACE2023540 | 0 | 0 | 0 | 100 | 0 | 0 | 0 |
| BRACE2023633 | 0 | 0 | 0 | 100 | 0 | 0 | 0 |
| BRACE2023718 | 0 | 0 | 0 | 11.391 | 0 | 0 | 0 |
| BRACE2023727 | 0 | 0 | 0 | 14.013 | 0 | 0 | 0 |
| BRACE2023744 | 0 | 0 | 0 | 100 | 0 | 0 | 0 |
| BRACE2023800 | 0 | 0 | 0 | 100 | 0 | 0 | 0 |
| BRACE2023801 | 0 | 0 | 0 | 26.645 | 0 | 0 | 0 |
| BRACE2024074 | 0 | 0 | 0 | 45.981 | 0 | 0 | 0 |
| BRACE2024222 | 0 | 0 | 0 | 100 | 0 | 0 | 0 |
| BRACE2024610 | 0 | 0 | 0 | 100 | 0 | 0 | 0 |
| BRACE2024627 | 0 | 0 | 0 | 8.689 | 0 | 0 | 0 |
| BRACE2024781 | 0 | 0 | 0 | 41.512 | 0 | 0 | 0 |
| BRACE2024826 | 0 | 0 | 0 | 100 | 0 | 0 | 0 |
| BRACE2025018 | 0 | 0 | 0 | 100 | 0 | 0 | 0 |
| BRACE2025316 | 0 | 0 | 0 | 100 | 0 | 0 | 0 |
| BRACE2025333 | 0 | 0 | 0 | 100 | 0 | 0 | 0 |
| BRACE2025437 | 0 | 0 | 0 | 2.167 | 0 | 0 | 0 |
| BRACE2025452 | 0 | 0 | 0 | 100 | 0 | 0 | 0 |
| BRACE2025492 | 0 | 0 | 0 | 18.680 | 0 | 0 | 0 |
| BRACE2025710 | 0 | 0 | 0 | 100 | 0 | 0 | 0 |
| BRACE2025796 | 0 | 0 | 0 | 22.162 | 0 | 0 | 0 |
| BRACE2025992 | 0 | 0 | 0 | 100 | 0 | 0 | 0 |
| BRACE2026054 | 0 | 0 | 0 | 16.225 | 0 | 0 | 0 |
| BRACE2026131 | 0 | 0 | 0 | 52.057 | 0 | 0 | 0 |
| BRACE2026141 | 0 | 0 | 0 | 27.755 | 0 | 0 | 0 |
| BRACE2026142 | 0 | 0 | 0 | 16.015 | 0 | 0 | 0 |
| BRACE2026293 | 0 | 0 | 0 | 100 | 0 | 0 | 0 |
| BRACE2026294 | 0 | 0 | 0 | 100 | 0 | 0 | 0 |
| BRACE2026404 | 0 | 0 | 0 | 100 | 0 | 0 | 0 |
| BRACE2026675 | 0 | 0 | 0 | 100 | 0 | 0 | 0 |
| BRACE2026725 | 0 | 0 | 0 | 13.014 | 0 | 0 | 0 |
| BRACE2026836 | 0 | 0 | 0 | 4.898 | 0 | 0 | 0 |
| BRACE2026920 | 0 | 0 | 0 | 100 | 0 | 0 | 0 |
| BRACE2026971 | 0 | 0 | 0 | 100 | 0 | 0 | 0 |
| BRACE2027018 | 0 | 0 | 0 | 16.691 | 0 | 0 | 0 |
| BRACE2027221 | 0 | 0 | 0 | 100 | 0 | 0 | 0 |
| BRACE2027258 | 0 | 0 | 0 | 23.981 | 0 | 34.044 | 0 |
| BRACE2027312 | 0 | 0 | 0 | 100 | 0 | 0 | 0 |
| BRACE2027382 | 0 | 0 | 0 | 100 | 0 | 0 | 0 |
| BRACE2027389 | 0 | 0 | 0 | 100 | 0 | 0 | 0 |
| BRACE2027408 | 0 | 0 | 0 | 100 | 0 | 0 | 0 |
| BRACE2027643 | 0 | 0 | 0 | 10.223 | 0 | 0 | 0 |
| BRACE2027767 | 0 | 0 | 0 | 41.512 | 0 | 0 | 0 |
| BRACE2027770 | 0 | 0 | 0 | 100 | 0 | 0 | 0 |
| BRACE2027879 | 0 | 0 | 0 | 100 | 0 | 0 | 0 |
| BRACE2027896 | 0 | 0 | 0 | 18.152 | 0 | 25.769 | 0 |
| BRACE2027970 | 0 | 0 | 0 | 45.981 | 0 | 0 | 0 |
| BRACE2028171 | 0 | 0 | 0 | 10.913 | 0 | 0 | 0 |
| BRACE2028392 | 0 | 0 | 0 | 7.236 | 0 | 0 | 0 |
| BRACE2028410 | 0 | 0 | 0 | 100 | 0 | 0 | 0 |
| BRACE2028636 | 0 | 0 | 0 | 37.993 | 48.354 | 0 | 0 |
| BRACE2028741 | 0 | 0 | 0 | 100 | 0 | 0 | 0 |
| BRACE2028956 | 0 | 0 | 0 | 100 | 0 | 0 | 0 |
| BRACE2028970 | 0 | 0 | 0 | 100 | 0 | 0 | 0 |
| BRACE2029112 | 0 | 0 | 0 | 100 | 0 | 0 | 0 |
| BRACE2029356 | 0 | 0 | 0 | 45.981 | 0 | 0 | 0 |
| BRACE2029396 | 0 | 0 | 0 | 100 | 0 | 0 | 0 |
| BRACE2029581 | 0 | 0 | 0 | 100 | 0 | 0 | 0 |
| BRACE2029644 | 0 | 0 | 0 | 45.981 | 0 | 0 | 0 |
| BRACE2029849 | 0 | 0 | 0 | 100 | 0 | 0 | 0 |
| BRACE2030039 | 0 | 0 | 0 | 100 | 0 | 0 | 0 |
| BRACE2030096 | 0 | 0 | 0 | 100 | 0 | 0 | 0 |
| BRACE2030323 | 0 | 0 | 0 | 33.350 | 0 | 31.563 | 0 |
| BRACE2030326 | 0 | 0 | 0 | 100 | 0 | 0 | 0 |
| BRACE2030341 | 0 | 0 | 0 | 4.909 | 0 | 6.969 | 0 |
| BRACE2030345 | 0 | 0 | 0 | 45.981 | 0 | 0 | 0 |
| BRACE2030736 | 0 | 0 | 0 | 100 | 0 | 0 | 0 |
| BRACE2030883 | 0 | 0 | 0 | 100 | 0 | 0 | 0 |
| BRACE2030884 | 0 | 0 | 0 | 100 | 0 | 0 | 0 |
| BRACE2031154 | 0 | 0 | 0 | 39.025 | 0 | 0 | 0 |
| BRACE2031232 | 0 | 0 | 0 | 100 | 0 | 0 | 0 |
| BRACE2031527 | 0 | 0 | 0 | 100 | 0 | 0 | 0 |
| BRACE2031531 | 0 | 0 | 0 | 100 | 0 | 0 | 0 |
| BRACE2031553 | 0 | 0 | 0 | 100 | 0 | 0 | 0 |
| BRACE2031899 | 0 | 0 | 0 | 100 | 0 | 0 | 0 |
| BRACE2032044 | 0 | 0 | 0 | 100 | 0 | 0 | 0 |
| BRACE2032090 | 0 | 0 | 0 | 100 | 0 | 0 | 0 |
| BRACE2032134 | 0 | 0 | 0 | 100 | 0 | 0 | 0 |
| BRACE2032148 | 0 | 0 | 0 | 100 | 0 | 0 | 0 |
| BRACE2032182 | 0 | 0 | 0 | 100 | 0 | 0 | 0 |
| BRACE2032329 | 0 | 0 | 0 | 100 | 0 | 0 | 0 |
| BRACE2032385 | 0 | 0 | 0 | 100 | 0 | 0 | 0 |
| BRACE2032436 | 0 | 0 | 0 | 100 | 0 | 0 | 0 |
| BRACE2032477 | 0 | 0 | 0 | 7.910 | 0 | 0 | 0 |
| BRACE2032502 | 0 | 0 | 0 | 39.025 | 0 | 0 | 0 |
| BRACE2032538 | 0 | 0 | 0 | 100 | 0 | 0 | 0 |
| BRACE2032584 | 0 | 0 | 0 | 100 | 0 | 0 | 0 |
| BRACE2032823 | 0 | 0 | 0 | 100 | 0 | 0 | 0 |
| BRACE2033128 | 0 | 0 | 0 | 100 | 0 | 0 | 0 |
| BRACE2033394 | 0 | 0 | 0 | 100 | 0 | 0 | 0 |
| BRACE2033478 | 0 | 0 | 0 | 100 | 0 | 0 | 0 |
| BRACE2033720 | 0 | 0 | 0 | 100 | 0 | 0 | 0 |
| BRACE2033786 | 0 | 0 | 0 | 100 | 0 | 0 | 0 |
| BRACE2034434 | 0 | 0 | 0 | 100 | 0 | 0 | 0 |
| BRACE2034523 | 0 | 0 | 0 | 100 | 0 | 0 | 0 |
| BRACE2034616 | 0 | 0 | 0 | 100 | 0 | 0 | 0 |
| BRACE2034622 | 0 | 0 | 0 | 100 | 0 | 0 | 0 |
| BRACE2034870 | 0 | 0 | 0 | 100 | 0 | 0 | 0 |
| BRACE2035003 | 0 | 0 | 0 | 100 | 0 | 0 | 0 |
| BRACE2035120 | 0 | 0 | 0 | 100 | 0 | 0 | 0 |
| BRACE2035124 | 0 | 0 | 0 | 100 | 0 | 0 | 0 |
| BRACE2035191 | 0 | 0 | 0 | 100 | 0 | 0 | 0 |
| BRACE2035278 | 0 | 0 | 0 | 53.329 | 0 | 0 | 0 |
| BRACE2035381 | 0 | 0 | 0 | 36.117 | 0 | 10.254 | 0 |
| BRACE2035441 | 0 | 0 | 0 | 100 | 0 | 0 | 0 |
| BRACE2035485 | 0 | 0 | 0 | 100 | 0 | 0 | 0 |
| BRACE2036005 | 0 | 0 | 0 | 100 | 0 | 0 | 0 |
| BRACE2036096 | 0 | 0 | 0 | 100 | 0 | 0 | 0 |
| BRACE2036743 | 0 | 0 | 0 | 100 | 0 | 0 | 0 |
| BRACE2036830 | 0 | 0 | 0 | 100 | 0 | 0 | 0 |
| BRACE2036834 | 0 | 0 | 0 | 100 | 0 | 0 | 0 |
| BRACE2036900 | 0 | 0 | 0 | 100 | 0 | 0 | 0 |
| BRACE2037132 | 0 | 0 | 0 | 41.512 | 0 | 0 | 0 |
| BRACE2037294 | 0 | 0 | 0 | 100 | 0 | 0 | 0 |
| BRACE2037295 | 0 | 0 | 0 | 2.328 | 0 | 0 | 0 |
| BRACE2037299 | 0 | 0 | 0 | 100 | 0 | 0 | 0 |
| BRACE2037310 | 0 | 0 | 0 | 27.851 | 0 | 0 | 0 |
| BRACE2037431 | 0 | 0 | 0 | 100 | 0 | 0 | 0 |
| BRACE2037692 | 0 | 0 | 0 | 100 | 0 | 0 | 0 |
| BRACE2037847 | 0 | 0 | 0 | 100 | 0 | 0 | 0 |
| BRACE2038114 | 0 | 0 | 0 | 100 | 0 | 0 | 0 |
| BRACE2038269 | 0 | 0 | 0 | 52.057 | 0 | 0 | 0 |
| BRACE2038271 | 0 | 0 | 0 | 100 | 0 | 0 | 0 |
| BRACE2038329 | 0 | 0 | 0 | 100 | 0 | 0 | 0 |
| BRACE2038492 | 0 | 0 | 0 | 100 | 0 | 0 | 0 |
| BRACE2038551 | 0 | 0 | 0 | 100 | 0 | 0 | 0 |
| BRACE2038618 | 0 | 0 | 0 | 100 | 0 | 0 | 0 |
| BRACE2039009 | 0 | 0 | 0 | 100 | 0 | 0 | 0 |
| BRACE2039128 | 0 | 0 | 0 | 100 | 0 | 0 | 0 |
| BRACE2039170 | 0 | 0 | 0 | 100 | 0 | 0 | 0 |
| BRACE2039249 | 0 | 0 | 0 | 100 | 0 | 0 | 0 |
| BRACE2039286 | 0 | 0 | 0 | 100 | 0 | 0 | 0 |
| BRACE2039297 | 0 | 0 | 0 | 100 | 0 | 0 | 0 |
| BRACE2039327 | 0 | 0 | 0 | 100 | 0 | 0 | 0 |
| BRACE2039362 | 0 | 0 | 0 | 100 | 0 | 0 | 0 |
| BRACE2039475 | 0 | 0 | 0 | 100 | 0 | 0 | 0 |
| BRACE2039600 | 0 | 0 | 0 | 100 | 0 | 0 | 0 |
| BRACE2039607 | 0 | 0 | 0 | 100 | 0 | 0 | 0 |
| BRACE2039624 | 0 | 0 | 0 | 0.687 | 0 | 1.950 | 0 |
| BRACE2039734 | 0 | 0 | 0 | 100 | 0 | 0 | 0 |
| BRACE2039823 | 0 | 0 | 0 | 41.329 | 0 | 58.671 | 0 |
| BRACE2039832 | 0 | 0 | 0 | 100 | 0 | 0 | 0 |
| BRACE2039893 | 0 | 0 | 0 | 100 | 0 | 0 | 0 |
| BRACE2039986 | 0 | 0 | 81.787 | 7.527 | 0 | 10.686 | 0 |
| BRACE2040138 | 0 | 0 | 0 | 100 | 0 | 0 | 0 |
| BRACE2040325 | 0 | 0 | 0 | 100 | 0 | 0 | 0 |
| BRACE2040514 | 0 | 0 | 0 | 100 | 0 | 0 | 0 |
| BRACE2040640 | 0 | 0 | 0 | 100 | 0 | 0 | 0 |
| BRACE2040987 | 0 | 0 | 0 | 100 | 0 | 0 | 0 |
| BRACE2041009 | 0 | 0 | 0 | 100 | 0 | 0 | 0 |
| BRACE2041095 | 0 | 0 | 0 | 100 | 0 | 0 | 0 |
| BRACE2041200 | 0 | 0 | 0 | 38.787 | 0 | 0 | 0 |
| BRACE2041264 | 0 | 0 | 0 | 100 | 0 | 0 | 0 |
| BRACE2041890 | 0 | 0 | 0 | 100 | 0 | 0 | 0 |
| BRACE2041898 | 0 | 0 | 0 | 100 | 0 | 0 | 0 |
| BRACE2042394 | 0 | 0 | 0 | 100 | 0 | 0 | 0 |
| BRACE2042398 | 0 | 0 | 0 | 100 | 0 | 0 | 0 |
| BRACE2042550 | 0 | 0 | 0 | 100 | 0 | 0 | 0 |
| BRACE2042620 | 0 | 0 | 0 | 100 | 0 | 0 | 0 |
| BRACE2042628 | 0 | 0 | 0 | 100 | 0 | 0 | 0 |
| BRACE2042873 | 0 | 0 | 0 | 100 | 0 | 0 | 0 |
| BRACE2043036 | 0 | 0 | 0 | 100 | 0 | 0 | 0 |
| BRACE2043105 | 0 | 0 | 0 | 100 | 0 | 0 | 0 |
| BRACE2043142 | 0 | 0 | 0 | 52.057 | 0 | 0 | 0 |
| BRACE2043248 | 0 | 0 | 0 | 100 | 0 | 0 | 0 |
| BRACE2043349 | 0 | 0 | 0 | 100 | 0 | 0 | 0 |
| BRACE2043381 | 0 | 0 | 0 | 7.639 | 0 | 0 | 0 |
| BRACE2043774 | 0 | 0 | 0 | 100 | 0 | 0 | 0 |
| BRACE2043942 | 0 | 0 | 0 | 100 | 0 | 0 | 0 |
| BRACE2044057 | 0 | 0 | 0 | 100 | 0 | 0 | 0 |
| BRACE2044105 | 0 | 0 | 0 | 14.283 | 0 | 0 | 0 |
| BRACE2044263 | 0 | 0 | 0 | 100 | 0 | 0 | 0 |
| BRACE2044286 | 0 | 0 | 0 | 26.499 | 0 | 0 | 0 |
| BRACE2044304 | 0 | 0 | 0 | 100 | 0 | 0 | 0 |
| BRACE2044473 | 0 | 0 | 0 | 100 | 0 | 0 | 0 |
| BRACE2044510 | 0 | 0 | 0 | 100 | 0 | 0 | 0 |
| BRACE2044640 | 0 | 0 | 0 | 100 | 0 | 0 | 0 |
| BRACE2044816 | 0 | 0 | 0 | 100 | 0 | 0 | 0 |
| BRACE2044946 | 0 | 0 | 0 | 100 | 0 | 0 | 0 |
| BRACE2044949 | 0 | 0 | 0 | 2.766 | 0 | 0 | 0 |
| BRACE2045300 | 0 | 0 | 0 | 100 | 0 | 0 | 0 |
| BRACE2045428 | 0 | 0 | 0 | 100 | 0 | 0 | 0 |
| BRACE2045445 | 0 | 0 | 0 | 37.308 | 0 | 0 | 0 |
| BRACE2045596 | 0 | 0 | 0 | 100 | 0 | 0 | 0 |
| BRACE2045772 | 0 | 0 | 0 | 2.055 | 0 | 0 | 0 |
| BRACE2045947 | 0 | 0 | 0 | 100 | 0 | 0 | 0 |
| BRACE2045954 | 0 | 0 | 0 | 38.766 | 0 | 0 | 0 |
| BRACE2046251 | 0 | 0 | 0 | 100 | 0 | 0 | 0 |
| BRACE2046295 | 0 | 0 | 0 | 100 | 0 | 0 | 0 |
| BRACE2046976 | 0 | 0 | 0 | 100 | 0 | 0 | 0 |
| BRACE2047011 | 0 | 0 | 0 | 100 | 0 | 0 | 0 |
| BRACE2047232 | 0 | 0 | 0 | 100 | 0 | 0 | 0 |
| BRACE2047350 | 0 | 0 | 0 | 45.981 | 0 | 0 | 0 |
| BRACE2047377 | 0 | 0 | 0 | 100 | 0 | 0 | 0 |
| BRACE2047385 | 0 | 0 | 0 | 100 | 0 | 0 | 0 |
| BRACE2047492 | 0 | 0 | 0 | 41.329 | 0 | 58.671 | 0 |
| BRACE2047558 | 0 | 0 | 0 | 100 | 0 | 0 | 0 |
| BRACE2047573 | 0 | 0 | 0 | 100 | 0 | 0 | 0 |
| BRACE2047835 | 0 | 0 | 0 | 100 | 0 | 0 | 0 |
| BRACE2047893 | 0 | 0 | 0 | 100 | 0 | 0 | 0 |
| BRACE2047975 | 0 | 0 | 0 | 100 | 0 | 0 | 0 |
| BRACE3000071 | 0 | 0 | 0 | 9.532 | 0 | 0 | 0 |
| BRACE3000404 | 0 | 0 | 0 | 100 | 0 | 0 | 0 |
| BRACE3000520 | 0 | 0 | 0 | 100 | 0 | 0 | 0 |
| BRACE3000697 | 0 | 0 | 0 | 100 | 0 | 0 | 0 |
| BRACE3000840 | 0 | 0 | 0 | 100 | 0 | 0 | 0 |
| BRACE3000973 | 0 | 0 | 0 | 28.699 | 0 | 13.581 | 0 |
| BRACE3001002 | 0 | 0 | 0 | 100 | 0 | 0 | 0 |
| BRACE3001058 | 0 | 0 | 0 | 100 | 0 | 0 | 0 |
| BRACE3001066 | 0 | 0 | 0 | 27.592 | 0 | 0 | 0 |
| BRACE3001113 | 0 | 0 | 0 | 18.680 | 0 | 0 | 0 |
| BRACE3001217 | 0 | 0 | 0 | 100 | 0 | 0 | 0 |
| BRACE3001299 | 0 | 0 | 0 | 100 | 0 | 0 | 0 |
| BRACE3001384 | 0 | 0 | 0 | 100 | 0 | 0 | 0 |
| BRACE3001391 | 0 | 0 | 0 | 100 | 0 | 0 | 0 |
| BRACE3001403 | 0 | 0 | 0 | 100 | 0 | 0 | 0 |
| BRACE3001595 | 0 | 0 | 0 | 100 | 0 | 0 | 0 |
| BRACE3001754 | 0 | 0 | 0 | 38.787 | 0 | 0 | 0 |
| BRACE3001948 | 0 | 0 | 0 | 100 | 0 | 0 | 0 |
| BRACE3001973 | 0 | 0 | 0 | 36.360 | 0 | 0 | 0 |
| BRACE3002184 | 0 | 0 | 0 | 19.020 | 0 | 0 | 0 |
| BRACE3002298 | 0 | 0 | 0 | 100 | 0 | 0 | 0 |
| BRACE3002344 | 0 | 0 | 0 | 100 | 0 | 0 | 0 |
| BRACE3002390 | 0 | 0 | 0 | 100 | 0 | 0 | 0 |
| BRACE3002420 | 0 | 0 | 0 | 100 | 0 | 0 | 0 |
| BRACE3002508 | 0 | 0 | 0 | 100 | 0 | 0 | 0 |
| BRACE3002541 | 0 | 0 | 0 | 100 | 0 | 0 | 0 |
| BRACE3002756 | 0 | 0 | 0 | 100 | 0 | 0 | 0 |
| BRACE3003004 | 0 | 0 | 0 | 100 | 0 | 0 | 0 |
| BRACE3003026 | 0 | 0 | 0 | 11.590 | 0 | 0 | 0 |
| BRACE3003053 | 0 | 0 | 0 | 36.360 | 0 | 0 | 0 |
| BRACE3003192 | 0 | 0 | 0 | 41.512 | 0 | 0 | 0 |
| BRACE3003413 | 0 | 0 | 0 | 45.981 | 0 | 0 | 0 |
| BRACE3003595 | 0 | 0 | 0 | 100 | 0 | 0 | 0 |
| BRACE3003698 | 0 | 0 | 0 | 45.981 | 0 | 0 | 0 |
| BRACE3003866 | 0 | 0 | 0 | 100 | 0 | 0 | 0 |
| BRACE3003920 | 0 | 0 | 0 | 100 | 0 | 0 | 0 |
| BRACE3004046 | 0 | 0 | 0 | 41.027 | 0 | 0 | 0 |
| BRACE3004058 | 0 | 0 | 0 | 100 | 0 | 0 | 0 |
| BRACE3004113 | 0 | 0 | 0 | 100 | 0 | 0 | 0 |
| BRACE3004150 | 0 | 0 | 0 | 2.757 | 0 | 0 | 0 |
| BRACE3004205 | 0 | 0 | 0 | 68.470 | 0 | 0 | 0 |
| BRACE3004358 | 0 | 0 | 0 | 100 | 0 | 0 | 0 |
| BRACE3004371 | 0 | 0 | 0 | 10.666 | 0 | 7.571 | 0 |
| BRACE3004435 | 0 | 0 | 0 | 100 | 0 | 0 | 0 |
| BRACE3004477 | 0 | 0 | 0 | 100 | 0 | 0 | 0 |
| BRACE3004767 | 0 | 0 | 0 | 41.512 | 0 | 0 | 0 |
| BRACE3004772 | 0 | 0 | 0 | 100 | 0 | 0 | 0 |
| BRACE3004783 | 0 | 0 | 0 | 100 | 0 | 0 | 0 |
| BRACE3004843 | 0 | 0 | 0 | 22.581 | 0 | 0 | 0 |
| BRACE3004862 | 0 | 0 | 0 | 38.787 | 0 | 0 | 0 |
| BRACE3004880 | 0 | 0 | 0 | 100 | 0 | 0 | 0 |
| BRACE3004887 | 0 | 0 | 0 | 100 | 0 | 0 | 0 |
| BRACE3004981 | 0 | 0 | 0 | 100 | 0 | 0 | 0 |
| BRACE3004996 | 0 | 0 | 0 | 100 | 0 | 0 | 0 |
| BRACE3004998 | 0 | 0 | 0 | 100 | 0 | 0 | 0 |
| BRACE3005103 | 0 | 0 | 0 | 100 | 0 | 0 | 0 |
| BRACE3005127 | 0 | 0 | 0 | 100 | 0 | 0 | 0 |
| BRACE3005145 | 0 | 0 | 0 | 100 | 0 | 0 | 0 |
| BRACE3005217 | 0 | 0 | 0 | 100 | 0 | 0 | 0 |
| BRACE3005225 | 0 | 0 | 0 | 100 | 0 | 0 | 0 |
| BRACE3005430 | 0 | 0 | 0 | 100 | 0 | 0 | 0 |
| BRACE3005499 | 0 | 0 | 0 | 22.199 | 0 | 0 | 0 |
| BRACE3005760 | 0 | 0 | 0 | 31.973 | 0 | 0 | 0 |
| BRACE3005870 | 0 | 0 | 0 | 100 | 0 | 0 | 0 |
| BRACE3005903 | 0 | 0 | 0 | 100 | 0 | 0 | 0 |
| BRACE3005938 | 0 | 0 | 0 | 3.908 | 0 | 1.110 | 0 |
| BRACE3005989 | 0 | 0 | 0 | 0.498 | 8.447 | 0 | 88.305 |
| BRACE3006113 | 0 | 0 | 0 | 100 | 0 | 0 | 0 |
| BRACE3006185 | 0 | 0 | 0 | 100 | 0 | 0 | 0 |
| BRACE3006226 | 0 | 0 | 0 | 100 | 0 | 0 | 0 |
| BRACE3006462 | 0 | 0 | 0 | 100 | 0 | 0 | 0 |
| BRACE3006463 | 0 | 0 | 0 | 100 | 0 | 0 | 0 |
| BRACE3006553 | 0 | 0 | 0 | 18.271 | 0 | 0 | 0 |
| BRACE3006872 | 0 | 0 | 0 | 100 | 0 | 0 | 0 |
| BRACE3006917 | 0 | 0 | 0 | 100 | 0 | 0 | 0 |
| BRACE3007058 | 0 | 0 | 0 | 52.057 | 0 | 0 | 0 |
| BRACE3007084 | 0 | 0 | 0 | 100 | 0 | 0 | 0 |
| BRACE3007085 | 0 | 0 | 0 | 100 | 0 | 0 | 0 |
| BRACE3007258 | 0 | 0 | 0 | 18.473 | 0 | 0 | 0 |
| BRACE3007322 | 0 | 0 | 0 | 100 | 0 | 0 | 0 |
| BRACE3007472 | 0 | 0 | 0 | 100 | 0 | 0 | 0 |
| BRACE3007480 | 0 | 0 | 0 | 13.820 | 0 | 6.540 | 0 |
| BRACE3007559 | 0 | 0 | 0 | 100 | 0 | 0 | 0 |
| BRACE3007625 | 0 | 0 | 0 | 100 | 0 | 0 | 0 |
| BRACE3007642 | 0 | 0 | 0 | 100 | 0 | 0 | 0 |
| BRACE3007649 | 0 | 0 | 0 | 100 | 0 | 0 | 0 |
| BRACE3007767 | 0 | 0 | 0 | 100 | 0 | 0 | 0 |
| BRACE3007869 | 0 | 0 | 0 | 100 | 0 | 0 | 0 |
| BRACE3008036 | 0 | 0 | 0 | 13.098 | 0 | 18.594 | 0 |
| BRACE3008066 | 0 | 0 | 0 | 100 | 0 | 0 | 0 |
| BRACE3008092 | 0 | 0 | 0 | 100 | 0 | 0 | 0 |
| BRACE3008095 | 0 | 0 | 0 | 45.981 | 0 | 0 | 0 |
| BRACE3008137 | 0 | 0 | 0 | 100 | 0 | 0 | 0 |
| BRACE3008255 | 0 | 0 | 0 | 100 | 0 | 0 | 0 |
| BRACE3008337 | 0 | 0 | 0 | 100 | 0 | 0 | 0 |
| BRACE3008384 | 0 | 0 | 0 | 56.141 | 0 | 0 | 0 |
| BRACE3008720 | 0 | 0 | 0 | 100 | 0 | 0 | 0 |
| BRACE3008772 | 0 | 0 | 0 | 37.308 | 0 | 0 | 0 |
| BRACE3009044 | 0 | 0 | 0 | 100 | 0 | 0 | 0 |
| BRACE3009075 | 0 | 0 | 0 | 100 | 0 | 0 | 0 |
| BRACE3009090 | 0 | 0 | 0 | 100 | 0 | 0 | 0 |
| BRACE3009127 | 0 | 0 | 0 | 100 | 0 | 0 | 0 |
| BRACE3009197 | 0 | 0 | 0 | 100 | 0 | 0 | 0 |
| BRACE3009237 | 0 | 0 | 0 | 12.875 | 0 | 0 | 0 |
| BRACE3009265 | 0 | 0 | 0 | 100 | 0 | 0 | 0 |
| BRACE3009297 | 0 | 0 | 0 | 100 | 0 | 0 | 0 |
| BRACE3009377 | 0 | 0 | 0 | 100 | 0 | 0 | 0 |
| BRACE3009392 | 0 | 0 | 0 | 100 | 0 | 0 | 0 |
| BRACE3009416 | 0 | 0 | 0 | 20.257 | 0 | 0 | 0 |
| BRACE3009539 | 0 | 0 | 0 | 100 | 0 | 0 | 0 |
| BRACE3009574 | 0 | 0 | 0 | 100 | 0 | 0 | 0 |
| BRACE3009701 | 0 | 0 | 0 | 100 | 0 | 0 | 0 |
| BRACE3009708 | 0 | 0 | 0 | 100 | 0 | 0 | 0 |
| BRACE3009724 | 0 | 0 | 0 | 41.512 | 0 | 0 | 0 |
| BRACE3009993 | 0 | 0 | 0 | 100 | 0 | 0 | 0 |
| BRACE3010076 | 0 | 0 | 0 | 100 | 0 | 0 | 0 |
| BRACE3010079 | 0 | 0 | 0 | 100 | 0 | 0 | 0 |
| BRACE3010086 | 0 | 0 | 0 | 100 | 0 | 0 | 0 |
| BRACE3010221 | 0 | 0 | 0 | 100 | 0 | 0 | 0 |
| BRACE3010283 | 0 | 0 | 0 | 38.787 | 0 | 0 | 0 |
| BRACE3010315 | 0 | 0 | 0 | 100 | 0 | 0 | 0 |
| BRACE3010397 | 0 | 0 | 0 | 24.243 | 0 | 0 | 0 |
| BRACE3010416 | 0 | 0 | 0 | 100 | 0 | 0 | 0 |
| BRACE3010428 | 0 | 0 | 0 | 3.250 | 0 | 3.460 | 0 |
| BRACE3010435 | 0 | 0 | 0 | 100 | 0 | 0 | 0 |
| BRACE3010438 | 0 | 0 | 0 | 5.084 | 0 | 1.804 | 0 |
| BRACE3010702 | 0 | 0 | 0 | 100 | 0 | 0 | 0 |
| BRACE3010862 | 0 | 0 | 0 | 100 | 0 | 0 | 0 |
| BRACE3011271 | 0 | 0 | 0 | 39.165 | 0 | 11.120 | 0 |
| BRACE3011421 | 0 | 0 | 0 | 40.103 | 0 | 0 | 0 |
| BRACE3011447 | 0 | 0 | 0 | 100 | 0 | 0 | 0 |
| BRACE3011505 | 0 | 0 | 0 | 100 | 0 | 0 | 0 |
| BRACE3011634 | 0 | 0 | 0 | 100 | 0 | 0 | 0 |
| BRACE3011774 | 0 | 0 | 0 | 100 | 0 | 0 | 0 |
| BRACE3012357 | 0 | 0 | 0 | 100 | 0 | 0 | 0 |
| BRACE3012574 | 0 | 0 | 0 | 100 | 0 | 0 | 0 |
| BRACE3012806 | 0 | 0 | 0 | 45.981 | 0 | 0 | 0 |
| BRACE3012930 | 0 | 0 | 0 | 38.787 | 0 | 0 | 0 |
| BRACE3013119 | 0 | 0 | 0 | 100 | 0 | 0 | 0 |
| BRACE3013418 | 0 | 0 | 0 | 100 | 0 | 0 | 0 |
| BRACE3013576 | 0 | 0 | 0 | 100 | 0 | 0 | 0 |
| BRACE3013740 | 0 | 0 | 0 | 100 | 0 | 0 | 0 |
| BRACE3013780 | 0 | 0 | 0 | 3.404 | 0 | 4.833 | 0 |
| BRACE3013874 | 0 | 0 | 0 | 100 | 0 | 0 | 0 |
| BRACE3013936 | 0 | 0 | 0 | 43.251 | 0 | 0 | 0 |
| BRACE3013986 | 0 | 0 | 0 | 100 | 0 | 0 | 0 |
| BRACE3014005 | 0 | 0 | 0 | 100 | 0 | 0 | 0 |
| BRACE3014068 | 0 | 0 | 0 | 100 | 0 | 0 | 0 |
| BRACE3014231 | 0 | 0 | 0 | 100 | 0 | 0 | 0 |
| BRACE3014290 | 0 | 0 | 0 | 100 | 0 | 0 | 0 |
| BRACE3014317 | 0 | 0 | 0 | 100 | 0 | 0 | 0 |
| BRACE3014523 | 0 | 0 | 0 | 100 | 0 | 0 | 0 |
| BRACE3014714 | 0 | 0 | 0 | 100 | 0 | 0 | 0 |
| BRACE3014807 | 0 | 0 | 0 | 100 | 0 | 0 | 0 |
| BRACE3014942 | 0 | 0 | 0 | 100 | 0 | 0 | 0 |
| BRACE3015090 | 0 | 0 | 0 | 100 | 0 | 0 | 0 |
| BRACE3015121 | 0 | 0 | 0 | 100 | 0 | 0 | 0 |
| BRACE3015262 | 0 | 0 | 0 | 100 | 0 | 0 | 0 |
| BRACE3015521 | 0 | 0 | 0 | 100 | 0 | 0 | 0 |
| BRACE3015808 | 0 | 0 | 0 | 100 | 0 | 0 | 0 |
| BRACE3015829 | 0 | 0 | 0 | 100 | 0 | 0 | 0 |
| BRACE3015894 | 0 | 0 | 0 | 100 | 0 | 0 | 0 |
| BRACE3015898 | 0 | 0 | 0 | 100 | 0 | 0 | 0 |
| BRACE3016020 | 0 | 0 | 0 | 100 | 0 | 0 | 0 |
| BRACE3016122 | 0 | 0 | 0 | 41.512 | 0 | 0 | 0 |
| BRACE3016167 | 0 | 0 | 0 | 100 | 0 | 0 | 0 |
| BRACE3016170 | 0 | 0 | 0 | 100 | 0 | 0 | 0 |
| BRACE3016580 | 0 | 0 | 0 | 100 | 0 | 0 | 0 |
| BRACE3016788 | 0 | 0 | 0 | 100 | 0 | 0 | 0 |
| BRACE3016810 | 0 | 0 | 0 | 100 | 0 | 0 | 0 |
| BRACE3016862 | 0 | 0 | 0 | 100 | 0 | 0 | 0 |
| BRACE3016884 | 0 | 0 | 0 | 9.165 | 0 | 0 | 0 |
| BRACE3017083 | 0 | 0 | 0 | 100 | 0 | 0 | 0 |
| BRACE3017253 | 0 | 0 | 0 | 55.894 | 0 | 0 | 0 |
| BRACE3018083 | 0 | 0 | 0 | 100 | 0 | 0 | 0 |
| BRACE3018308 | 0 | 0 | 0 | 100 | 0 | 0 | 0 |
| BRACE3018963 | 0 | 0 | 0 | 41.027 | 0 | 0 | 0 |
| BRACE3019055 | 0 | 0 | 0 | 100 | 0 | 0 | 0 |
| BRACE3019071 | 0 | 0 | 0 | 40.231 | 0 | 0 | 0 |
| BRACE3019084 | 0 | 0 | 0 | 100 | 0 | 0 | 0 |
| BRACE3019570 | 0 | 0 | 0 | 100 | 0 | 0 | 0 |
| BRACE3019611 | 0 | 0 | 0 | 100 | 0 | 0 | 0 |
| BRACE3019817 | 0 | 0 | 0 | 100 | 0 | 0 | 0 |
| BRACE3019828 | 0 | 0 | 0 | 100 | 0 | 0 | 0 |
| BRACE3019941 | 0 | 0 | 0 | 2.074 | 0 | 0 | 0 |
| BRACE3020151 | 0 | 0 | 0 | 100 | 0 | 0 | 0 |
| BRACE3020194 | 0 | 0 | 0 | 100 | 0 | 0 | 0 |
| BRACE3020286 | 0 | 0 | 0 | 100 | 0 | 0 | 0 |
| BRACE3020356 | 0 | 0 | 0 | 100 | 0 | 0 | 0 |
| BRACE3020594 | 0 | 0 | 0 | 100 | 0 | 0 | 0 |
| BRACE3020669 | 0 | 0 | 0 | 38.787 | 0 | 0 | 0 |
| BRACE3020671 | 0 | 0 | 0 | 100 | 0 | 0 | 0 |
| BRACE3020890 | 0 | 0 | 0 | 100 | 0 | 0 | 0 |
| BRACE3021148 | 0 | 0 | 0 | 100 | 0 | 0 | 0 |
| BRACE3021430 | 0 | 83.734 | 0 | 8.467 | 0 | 0 | 0 |
| BRACE3021517 | 0 | 0 | 0 | 100 | 0 | 0 | 0 |
| BRACE3021805 | 0 | 0 | 0 | 100 | 0 | 0 | 0 |
| BRACE3022051 | 0 | 0 | 0 | 100 | 0 | 0 | 0 |
| BRACE3022303 | 0 | 0 | 0 | 100 | 0 | 0 | 0 |
| BRACE3022312 | 0 | 0 | 0 | 24.151 | 0 | 0 | 0 |
| BRACE3022340 | 0 | 0 | 0 | 41.512 | 0 | 0 | 0 |
| BRACE3022847 | 0 | 0 | 0 | 100 | 0 | 0 | 0 |
| BRACE3023604 | 0 | 0 | 0 | 100 | 0 | 0 | 0 |
| BRACE3023912 | 0 | 0 | 0 | 100 | 0 | 0 | 0 |
| BRACE3024073 | 0 | 0 | 0 | 100 | 0 | 0 | 0 |
| BRACE3024359 | 0 | 0 | 0 | 100 | 0 | 0 | 0 |
| BRACE3024379 | 0 | 0 | 0 | 100 | 0 | 0 | 0 |
| BRACE3024444 | 0 | 0 | 0 | 100 | 0 | 0 | 0 |
| BRACE3024497 | 0 | 0 | 0 | 100 | 0 | 0 | 0 |
| BRACE3024537 | 0 | 0 | 0 | 100 | 0 | 0 | 0 |
| BRACE3024659 | 0 | 0 | 0 | 100 | 0 | 0 | 0 |
| BRACE3024662 | 0 | 0 | 0 | 100 | 0 | 0 | 0 |
| BRACE3024879 | 0 | 0 | 0 | 100 | 0 | 0 | 0 |
| BRACE3025153 | 0 | 0 | 0 | 100 | 0 | 0 | 0 |
| BRACE3025302 | 0 | 0 | 0 | 100 | 0 | 0 | 0 |
| BRACE3025457 | 0 | 0 | 0 | 100 | 0 | 0 | 0 |
| BRACE3025531 | 0 | 0 | 0 | 100 | 0 | 0 | 0 |
| BRACE3025627 | 0 | 0 | 0 | 100 | 0 | 0 | 0 |
| BRACE3025630 | 0 | 0 | 0 | 100 | 0 | 0 | 0 |
| BRACE3025719 | 0 | 0 | 0 | 12.784 | 0 | 0 | 0 |
| BRACE3026008 | 0 | 0 | 0 | 100 | 0 | 0 | 0 |
| BRACE3026075 | 0 | 0 | 0 | 100 | 0 | 0 | 0 |
| BRACE3026161 | 0 | 0 | 0 | 100 | 0 | 0 | 0 |
| BRACE3026290 | 0 | 0 | 0 | 100 | 0 | 0 | 0 |
| BRACE3026345 | 0 | 0 | 0 | 100 | 0 | 0 | 0 |
| BRACE3026456 | 0 | 0 | 0 | 100 | 0 | 0 | 0 |
| BRACE3026735 | 0 | 0 | 0 | 51.471 | 0 | 24.356 | 0 |
| BRACE3026802 | 0 | 0 | 0 | 36.390 | 0 | 0 | 0 |
| BRACE3026844 | 0 | 0 | 0 | 100 | 0 | 0 | 0 |
| BRACE3026947 | 0 | 0 | 0 | 100 | 0 | 0 | 0 |
| BRACE3026993 | 0 | 0 | 0 | 100 | 0 | 0 | 0 |
| BRACE3027242 | 0 | 0 | 0 | 100 | 0 | 0 | 0 |
| BRACE3027256 | 0 | 0 | 0 | 100 | 0 | 0 | 0 |
| BRACE3027326 | 0 | 0 | 0 | 100 | 0 | 0 | 0 |
| BRACE3027478 | 0 | 0 | 0 | 11.360 | 0 | 16.126 | 0 |
| BRACE3027480 | 0 | 0 | 0 | 100 | 0 | 0 | 0 |
| BRACE3027931 | 0 | 0 | 0 | 23.879 | 0 | 0 | 0 |
| BRACE3027976 | 0 | 0 | 0 | 45.981 | 0 | 0 | 0 |
| BRACE3028214 | 0 | 0 | 0 | 100 | 0 | 0 | 0 |
| BRACE3028241 | 0 | 0 | 0 | 100 | 0 | 0 | 0 |
| BRACE3028360 | 0 | 0 | 0 | 100 | 0 | 0 | 0 |
| BRACE3028895 | 0 | 0 | 0 | 32.302 | 0 | 0 | 0 |
| BRACE3028998 | 0 | 0 | 0 | 100 | 0 | 0 | 0 |
| BRACE3029005 | 0 | 0 | 0 | 100 | 0 | 0 | 0 |
| BRACE3029021 | 0 | 0 | 0 | 25.807 | 0 | 0 | 0 |
| BRACE3029205 | 0 | 0 | 0 | 100 | 0 | 0 | 0 |
| BRACE3029447 | 0 | 0 | 0 | 100 | 0 | 0 | 0 |
| BRACE3030057 | 0 | 0 | 0 | 100 | 0 | 0 | 0 |
| BRACE3030103 | 0 | 0 | 0 | 100 | 0 | 0 | 0 |
| BRACE3030538 | 0 | 0 | 0 | 36.360 | 0 | 0 | 0 |
| BRACE3030748 | 0 | 0 | 0 | 100 | 0 | 0 | 0 |
| BRACE3030866 | 0 | 0 | 0 | 41.512 | 0 | 0 | 0 |
| BRACE3031161 | 0 | 0 | 0 | 100 | 0 | 0 | 0 |
| BRACE3031184 | 0 | 0 | 0 | 100 | 0 | 0 | 0 |
| BRACE3031185 | 0 | 0 | 0 | 41.512 | 0 | 0 | 0 |
| BRACE3031315 | 0 | 0 | 0 | 41.329 | 0 | 58.671 | 0 |
| BRACE3031372 | 0 | 0 | 0 | 100 | 0 | 0 | 0 |
| BRACE3031579 | 0 | 0 | 0 | 100 | 0 | 0 | 0 |
| BRACE3031728 | 0 | 0 | 0 | 100 | 0 | 0 | 0 |
| BRACE3031838 | 0 | 0 | 0 | 41.512 | 0 | 0 | 0 |
| BRACE3031843 | 0 | 0 | 0 | 100 | 0 | 0 | 0 |
| BRACE3032427 | 0 | 0 | 0 | 41.512 | 0 | 0 | 0 |
| BRACE3032455 | 0 | 0 | 0 | 100 | 0 | 0 | 0 |
| BRACE3032537 | 0 | 0 | 0 | 100 | 0 | 0 | 0 |
| BRACE3032538 | 0 | 0 | 0 | 100 | 0 | 0 | 0 |
| BRACE3032631 | 0 | 0 | 0 | 8.951 | 0 | 0 | 0 |
| BRACE3032771 | 0 | 0 | 0 | 100 | 0 | 0 | 0 |
| BRACE3032980 | 0 | 0 | 0 | 100 | 0 | 0 | 0 |
| BRACE3032983 | 0 | 0 | 0 | 100 | 0 | 0 | 0 |
| BRACE3033525 | 0 | 0 | 0 | 100 | 0 | 0 | 0 |
| BRACE3034159 | 0 | 0 | 0 | 100 | 0 | 0 | 0 |
| BRACE3034183 | 0 | 0 | 0 | 100 | 0 | 0 | 0 |
| BRACE3034389 | 0 | 0 | 0 | 100 | 0 | 0 | 0 |
| BRACE3034964 | 0 | 0 | 0 | 100 | 0 | 0 | 0 |
| BRACE3034993 | 0 | 0 | 0 | 100 | 0 | 0 | 0 |
| BRACE3035168 | 0 | 0 | 0 | 29.854 | 0 | 0 | 0 |
| BRACE3035227 | 0 | 0 | 0 | 100 | 0 | 0 | 0 |
| BRACE3036156 | 0 | 0 | 0 | 31.069 | 0 | 0 | 0 |
| BRACE3036271 | 0 | 0 | 0 | 100 | 0 | 0 | 0 |
| BRACE3037612 | 0 | 0 | 0 | 100 | 0 | 0 | 0 |
| BRACE3037637 | 0 | 0 | 0 | 100 | 0 | 0 | 0 |
| BRACE3037803 | 0 | 0 | 0 | 100 | 0 | 0 | 0 |
| BRACE3038012 | 0 | 0 | 0 | 100 | 0 | 0 | 0 |
| BRACE3038030 | 0 | 0 | 0 | 100 | 0 | 0 | 0 |
| BRACE3038569 | 0 | 0 | 0 | 100 | 0 | 0 | 0 |
| BRACE3038570 | 0 | 0 | 0 | 100 | 0 | 0 | 0 |
| BRACE3038687 | 0 | 0 | 0 | 100 | 0 | 0 | 0 |
| BRACE3038760 | 0 | 0 | 0 | 100 | 0 | 0 | 0 |
| BRACE3039288 | 0 | 0 | 0 | 100 | 0 | 0 | 0 |
| BRACE3039358 | 0 | 0 | 0 | 14.668 | 0 | 0 | 0 |
| BRACE3039378 | 0 | 0 | 0 | 100 | 0 | 0 | 0 |
| BRACE3039454 | 0 | 0 | 0 | 100 | 0 | 0 | 0 |
| BRACE3040012 | 0 | 0 | 0 | 100 | 0 | 0 | 0 |
| BRACE3040239 | 0 | 0 | 0 | 100 | 0 | 0 | 0 |
| BRACE3040504 | 0 | 0 | 0 | 100 | 0 | 0 | 0 |
| BRACE3040644 | 0 | 0 | 0 | 100 | 0 | 0 | 0 |
| BRACE3040856 | 0 | 0 | 0 | 100 | 0 | 0 | 0 |
| BRACE3040863 | 0 | 0 | 0 | 12.696 | 0 | 0 | 0 |
| BRACE3041059 | 0 | 0 | 0 | 100 | 0 | 0 | 0 |
| BRACE3041162 | 0 | 0 | 0 | 100 | 0 | 0 | 0 |
| BRACE3041827 | 0 | 0 | 0 | 100 | 0 | 0 | 0 |
| BRACE3042046 | 0 | 0 | 0 | 100 | 0 | 0 | 0 |
| BRACE3042210 | 0 | 0 | 0 | 100 | 0 | 0 | 0 |
| BRACE3042326 | 0 | 0 | 0 | 24.903 | 0 | 0 | 0 |
| BRACE3042409 | 0 | 0 | 0 | 100 | 0 | 0 | 0 |
| BRACE3042432 | 0 | 0 | 0 | 41.512 | 0 | 0 | 0 |
| BRACE3042594 | 0 | 0 | 0 | 100 | 0 | 0 | 0 |
| BRACE3043597 | 0 | 0 | 0 | 100 | 0 | 0 | 0 |
| BRACE3044090 | 0 | 0 | 0 | 100 | 0 | 0 | 0 |
| BRACE3044172 | 0 | 0 | 0 | 100 | 0 | 0 | 0 |
| BRACE3044247 | 0 | 0 | 0 | 100 | 0 | 0 | 0 |
| BRACE3044377 | 0 | 0 | 0 | 100 | 0 | 0 | 0 |
| BRACE3044389 | 0 | 0 | 0 | 100 | 0 | 0 | 0 |
| BRACE3044495 | 0 | 0 | 0 | 100 | 0 | 0 | 0 |
| BRACE3045011 | 0 | 0 | 0 | 100 | 0 | 0 | 0 |
| BRACE3045033 | 0 | 0 | 0 | 100 | 0 | 0 | 0 |
| BRACE3045078 | 0 | 0 | 0 | 58.183 | 0 | 0 | 0 |
| BRACE3045145 | 0 | 0 | 0 | 100 | 0 | 0 | 0 |
| BRACE3045424 | 0 | 0 | 0 | 100 | 0 | 0 | 0 |
| BRACE3045708 | 0 | 0 | 0 | 100 | 0 | 0 | 0 |
| BRACE3045981 | 0 | 0 | 0 | 56.286 | 0 | 0 | 0 |
| BRACE3046049 | 0 | 0 | 0 | 100 | 0 | 0 | 0 |
| BRACE3046152 | 0 | 0 | 0 | 100 | 0 | 0 | 0 |
| BRACE3046294 | 0 | 0 | 0 | 100 | 0 | 0 | 0 |
| BRACE3046450 | 0 | 0 | 0 | 78.016 | 0 | 0 | 0 |
| BRACE3046466 | 0 | 0 | 0 | 100 | 0 | 0 | 0 |
| BRACE3046491 | 0 | 0 | 0 | 100 | 0 | 0 | 0 |
| BRACE3046609 | 0 | 0 | 0 | 100 | 0 | 0 | 0 |
| BRACE3046762 | 0 | 0 | 0 | 100 | 0 | 0 | 0 |
| BRACE3046837 | 0 | 0 | 0 | 100 | 0 | 0 | 0 |
| BRACE3046855 | 0 | 0 | 0 | 100 | 0 | 0 | 0 |
| BRACE3046966 | 0 | 0 | 0 | 100 | 0 | 0 | 0 |
| BRACE3047018 | 0 | 0 | 0 | 100 | 0 | 0 | 0 |
| BRACE3047482 | 0 | 0 | 0 | 100 | 0 | 0 | 0 |
| BRACE3047801 | 0 | 0 | 0 | 100 | 0 | 0 | 0 |
| BRALZ1000047 | 0 | 0 | 0 | 0 | 100 | 0 | 0 |
| BRALZ1000103 | 0 | 0 | 0 | 0 | 100 | 0 | 0 |
| BRALZ2000189 | 0 | 0 | 0 | 0 | 100 | 0 | 0 |
| BRALZ2000263 | 0 | 0 | 0 | 16.418 | 83.582 | 0 | 0 |
| BRALZ2000988 | 0 | 0 | 0 | 25.787 | 18.754 | 0 | 0 |
| BRALZ2001038 | 0 | 0 | 0 | 23.070 | 58.725 | 0 | 0 |
| BRALZ2001279 | 0 | 0 | 0 | 0 | 100 | 0 | 0 |
| BRALZ2001350 | 0 | 0 | 0 | 0 | 100 | 0 | 0 |
| BRALZ2001445 | 0 | 0 | 0 | 0 | 100 | 0 | 0 |
| BRALZ2001559 | 0 | 0 | 0 | 0 | 100 | 0 | 0 |
| BRALZ2001743 | 0 | 0 | 0 | 0 | 100 | 0 | 0 |
| BRALZ2001834 | 0 | 0 | 0 | 0 | 76.517 | 0 | 0 |
| BRALZ2002174 | 0 | 0 | 0 | 0 | 100 | 0 | 0 |
| BRALZ2002418 | 0 | 0 | 0 | 0 | 78.195 | 21.805 | 0 |
| BRALZ2002477 | 0 | 0 | 0 | 0 | 100 | 0 | 0 |
| BRALZ2003119 | 0 | 0 | 0 | 0 | 100 | 0 | 0 |
| BRALZ2003330 | 0 | 0 | 0 | 0 | 100 | 0 | 0 |
| BRALZ2003453 | 0 | 0 | 0 | 0 | 100 | 0 | 0 |
| BRALZ2003525 | 0 | 0 | 0 | 0 | 100 | 0 | 0 |
| BRALZ2004397 | 0 | 0 | 0 | 0 | 100 | 0 | 0 |
| BRALZ2005467 | 0 | 0 | 0 | 0 | 100 | 0 | 0 |
| BRALZ2005471 | 0 | 0 | 0 | 0 | 100 | 0 | 0 |
| BRALZ2005888 | 0 | 0 | 0 | 0 | 100 | 0 | 0 |
| BRALZ2005950 | 0 | 0 | 0 | 0 | 100 | 0 | 0 |
| BRALZ2005992 | 0 | 0 | 0 | 0 | 100 | 0 | 0 |
| BRALZ2006288 | 0 | 0 | 0 | 0 | 39.404 | 10.988 | 0 |
| BRALZ2006474 | 0 | 0 | 0 | 0 | 100 | 0 | 0 |
| BRALZ2006560 | 0 | 0 | 0 | 0 | 100 | 0 | 0 |
| BRALZ2006734 | 0 | 0 | 0 | 0 | 100 | 0 | 0 |
| BRALZ2006800 | 0 | 0 | 0 | 0 | 100 | 0 | 0 |
| BRALZ2006976 | 0 | 0 | 0 | 0 | 100 | 0 | 0 |
| BRALZ2007376 | 0 | 0 | 0 | 0 | 100 | 0 | 0 |
| BRALZ2007545 | 0 | 0 | 0 | 0 | 100 | 0 | 0 |
| BRALZ2007576 | 0 | 0 | 0 | 0 | 100 | 0 | 0 |
| BRALZ2007661 | 0 | 0 | 0 | 0 | 100 | 0 | 0 |
| BRALZ2007790 | 0 | 0 | 0 | 0 | 100 | 0 | 0 |
| BRALZ2007793 | 0 | 0 | 0 | 0 | 100 | 0 | 0 |
| BRALZ2007952 | 0 | 0 | 0 | 0 | 100 | 0 | 0 |
| BRALZ2008031 | 0 | 0 | 0 | 0 | 100 | 0 | 0 |
| BRALZ2008166 | 0 | 0 | 0 | 0 | 66.169 | 0 | 0 |
| BRALZ2008462 | 0 | 0 | 0 | 0 | 100 | 0 | 0 |
| BRALZ2008617 | 0 | 56.870 | 0 | 5.751 | 29.277 | 0 | 0 |
| BRALZ2008696 | 0 | 0 | 0 | 0 | 78.324 | 0 | 0 |
| BRALZ2008763 | 0 | 0 | 0 | 0 | 100 | 0 | 0 |
| BRALZ2008869 | 0 | 0 | 0 | 0 | 100 | 0 | 0 |
| BRALZ2008930 | 0 | 0 | 0 | 0 | 100 | 0 | 0 |
| BRALZ2009446 | 0 | 32.727 | 0 | 0 | 16.848 | 0 | 0 |
| BRALZ2009482 | 0 | 2.608 | 0 | 0.528 | 1.343 | 1.123 | 0 |
| BRALZ2009486 | 0 | 0 | 0 | 0 | 75.903 | 0 | 0 |
| BRALZ2010186 | 0 | 0 | 0 | 0 | 100 | 0 | 0 |
| BRALZ2010842 | 0 | 0 | 0 | 0 | 100 | 0 | 0 |
| BRALZ2011337 | 0 | 0 | 0 | 0 | 100 | 0 | 0 |
| BRALZ2011796 | 0 | 0 | 0 | 3.912 | 39.830 | 0 | 0 |
| BRALZ2011880 | 0 | 21.400 | 0 | 2.164 | 22.034 | 3.072 | 0 |
| BRALZ2012050 | 0 | 0 | 0 | 0 | 100 | 0 | 0 |
| BRALZ2012183 | 0 | 0 | 0 | 0 | 100 | 0 | 0 |
| BRALZ2012848 | 0 | 0 | 0 | 0 | 100 | 0 | 0 |
| BRALZ2013621 | 0 | 0 | 0 | 0 | 100 | 0 | 0 |
| BRALZ2013690 | 0 | 0 | 0 | 0 | 100 | 0 | 0 |
| BRALZ2014054 | 0 | 0 | 0 | 0 | 38.408 | 0 | 0 |
| BRALZ2014316 | 0 | 0 | 0 | 0 | 100 | 0 | 0 |
| BRALZ2014484 | 0 | 0 | 0 | 0 | 100 | 0 | 0 |
| BRALZ2014951 | 0 | 0 | 0 | 0 | 100 | 0 | 0 |
| BRALZ2015168 | 0 | 0 | 0 | 0 | 100 | 0 | 0 |
| BRALZ2015811 | 0 | 0 | 0 | 0 | 100 | 0 | 0 |
| BRALZ2016085 | 0 | 0 | 0 | 0 | 100 | 0 | 0 |
| BRALZ2016498 | 0 | 0 | 0 | 0 | 100 | 0 | 0 |
| BRALZ2017359 | 0 | 0 | 0 | 0 | 75.184 | 0 | 0 |
| BRALZ2017531 | 0 | 0 | 0 | 0 | 100 | 0 | 0 |
| BRALZ2017607 | 0 | 0 | 0 | 0 | 100 | 0 | 0 |
| BRALZ2017620 | 0 | 0 | 0 | 0 | 100 | 0 | 0 |
| BRALZ2017844 | 0 | 0 | 0 | 0 | 49.396 | 0 | 0 |
| BRALZ2018342 | 0 | 0 | 0 | 0 | 100 | 0 | 0 |
| BRALZ2018492 | 0 | 0 | 0 | 16.418 | 83.582 | 0 | 0 |
| BRALZ2018834 | 0 | 0 | 0 | 0 | 22.547 | 6.287 | 0 |
| BRALZ2019047 | 0 | 0 | 0 | 0 | 76.517 | 0 | 0 |
| BRAMY1000095 | 0 | 0 | 0 | 0 | 0 | 100 | 0 |
| BRAMY1000098 | 0 | 0 | 0 | 0 | 0 | 100 | 0 |
| BRAMY1000130 | 0 | 0 | 0 | 0 | 0 | 12.137 | 0 |
| BRAMY1000157 | 0 | 0 | 0 | 0 | 0 | 100 | 0 |
| BRAMY1000173 | 0 | 0 | 0 | 0 | 0 | 21.311 | 0 |
| BRAMY2000021 | 0 | 0 | 0 | 0 | 0 | 100 | 0 |
| BRAMY2000025 | 0 | 0 | 0 | 0 | 0 | 47.699 | 0 |
| BRAMY2000052 | 0 | 0 | 0 | 0 | 39.130 | 21.823 | 0 |
| BRAMY2000076 | 0 | 0 | 0 | 0 | 0 | 100 | 0 |
| BRAMY2000086 | 0 | 0 | 0 | 0 | 0 | 100 | 0 |
| BRAMY2000151 | 0 | 0 | 0 | 0 | 0 | 22.213 | 0 |
| BRAMY2000181 | 0 | 0 | 0 | 0 | 0 | 35.292 | 0 |
| BRAMY2000196 | 0 | 0 | 0 | 0 | 0 | 100 | 0 |
| BRAMY2000231 | 0 | 0 | 0 | 0 | 0 | 36.677 | 0 |
| BRAMY2000277 | 0 | 0 | 0 | 0 | 0 | 100 | 0 |
| BRAMY2000354 | 0 | 0 | 0 | 1.951 | 0 | 2.769 | 0 |
| BRAMY2000388 | 0 | 0 | 0 | 8.685 | 0 | 12.329 | 0 |
| BRAMY2000411 | 0 | 0 | 0 | 4.399 | 0 | 18.735 | 0 |
| BRAMY2000508 | 0 | 0 | 0 | 0 | 0 | 35.461 | 0 |
| BRAMY2000562 | 0 | 0 | 0 | 1.364 | 6.944 | 9.681 | 0 |
| BRAMY2000585 | 0 | 0 | 0 | 0 | 0 | 2.475 | 0 |
| BRAMY2000653 | 0 | 0 | 0 | 0 | 0 | 100 | 0 |
| BRAMY2000814 | 0 | 0 | 0 | 0 | 0 | 21.396 | 0 |
| BRAMY2000893 | 0 | 10.681 | 0 | 0 | 0 | 1.533 | 0 |
| BRAMY2000946 | 0 | 0 | 0 | 0 | 0 | 100 | 0 |
| BRAMY2000981 | 0 | 0 | 0 | 0 | 0 | 100 | 0 |
| BRAMY2000987 | 0 | 0 | 0 | 24.093 | 0 | 17.102 | 0 |
| BRAMY2001068 | 0 | 0 | 0 | 0 | 0 | 100 | 0 |
| BRAMY2001114 | 0 | 0 | 0 | 0 | 0 | 100 | 0 |
| BRAMY2001203 | 0 | 0 | 0 | 0 | 0 | 100 | 0 |
| BRAMY2001282 | 0 | 0 | 0 | 0 | 0 | 100 | 0 |
| BRAMY2001367 | 0 | 0 | 0 | 0 | 0 | 100 | 0 |
| BRAMY2001427 | 0 | 0 | 0 | 0 | 0 | 100 | 0 |
| BRAMY2001473 | 0 | 0 | 0 | 0 | 0 | 100 | 0 |
| BRAMY2001678 | 0 | 0 | 0 | 0 | 0 | 37.663 | 0 |
| BRAMY2002005 | 0 | 0 | 0 | 0 | 0 | 100 | 0 |
| BRAMY2002044 | 0 | 0 | 0 | 0 | 0 | 100 | 0 |
| BRAMY2002072 | 0 | 0 | 0 | 0 | 0 | 100 | 0 |
| BRAMY2002158 | 0 | 0 | 0 | 0 | 0 | 100 | 0 |
| BRAMY2002311 | 0 | 0 | 0 | 0 | 0 | 100 | 0 |
| BRAMY2002339 | 0 | 0 | 0 | 0 | 0 | 100 | 0 |
| BRAMY2002364 | 0 | 0 | 0 | 0 | 0 | 100 | 0 |
| BRAMY2002369 | 0 | 0 | 0 | 0 | 0 | 8.220 | 0 |
| BRAMY2002479 | 0 | 0 | 0 | 0 | 0 | 21.841 | 0 |
| BRAMY2002493 | 0 | 0 | 0 | 0 | 0 | 100 | 0 |
| BRAMY2002525 | 0 | 0 | 0 | 41.329 | 0 | 58.671 | 0 |
| BRAMY2002584 | 0 | 0 | 0 | 0 | 0 | 100 | 0 |
| BRAMY2002604 | 0 | 0 | 0 | 0 | 0 | 100 | 0 |
| BRAMY2002621 | 0 | 0 | 0 | 25.113 | 0 | 35.650 | 0 |
| BRAMY2002638 | 0 | 0 | 0 | 0 | 0 | 100 | 0 |
| BRAMY2002739 | 0 | 0 | 0 | 0 | 0 | 100 | 0 |
| BRAMY2002799 | 0 | 0 | 0 | 0 | 0 | 50.189 | 0 |
| BRAMY2002853 | 0 | 0 | 0 | 0 | 0 | 100 | 0 |
| BRAMY2002862 | 0 | 0 | 0 | 0 | 0 | 100 | 0 |
| BRAMY2002956 | 0 | 0 | 0 | 0 | 0 | 100 | 0 |
| BRAMY2003008 | 0 | 0 | 0 | 0 | 0 | 26.646 | 0 |
| BRAMY2003037 | 0 | 0 | 0 | 0 | 0 | 100 | 0 |
| BRAMY2003117 | 0 | 0 | 0 | 0 | 0 | 100 | 0 |
| BRAMY2003287 | 0 | 0 | 0 | 0 | 0 | 100 | 0 |
| BRAMY2003325 | 0 | 0 | 0 | 0 | 0 | 12.511 | 0 |
| BRAMY2003529 | 0 | 0 | 0 | 0 | 0 | 100 | 0 |
| BRAMY2003538 | 0 | 0 | 0 | 26.047 | 0 | 73.953 | 0 |
| BRAMY2003585 | 0 | 0 | 0 | 0 | 0 | 100 | 0 |
| BRAMY2003634 | 0 | 0 | 0 | 0 | 0 | 100 | 0 |
| BRAMY2003653 | 0 | 0 | 0 | 0 | 0 | 100 | 0 |
| BRAMY2003681 | 0 | 0 | 0 | 0 | 0 | 100 | 0 |
| BRAMY2003893 | 0 | 0 | 0 | 0 | 0 | 100 | 0 |
| BRAMY2003897 | 0 | 0 | 0 | 0 | 0 | 100 | 0 |
| BRAMY2003898 | 0 | 0 | 0 | 0 | 0 | 100 | 0 |
| BRAMY2003904 | 0 | 0 | 0 | 0 | 0 | 100 | 0 |
| BRAMY2003926 | 0 | 0 | 0 | 0 | 0 | 100 | 0 |
| BRAMY2003929 | 0 | 0 | 0 | 0 | 0 | 21.427 | 0 |
| BRAMY2004031 | 0 | 0 | 0 | 0 | 0 | 12.812 | 0 |
| BRAMY2004058 | 0 | 0 | 0 | 0 | 0 | 100 | 0 |
| BRAMY2004183 | 0 | 0 | 0 | 0 | 0 | 100 | 0 |
| BRAMY2004335 | 0 | 0 | 0 | 0 | 0 | 100 | 0 |
| BRAMY2004351 | 0 | 0 | 0 | 0 | 0 | 100 | 0 |
| BRAMY2004352 | 0 | 0 | 0 | 0 | 0 | 100 | 0 |
| BRAMY2004363 | 0 | 0 | 0 | 1.451 | 0 | 4.120 | 0 |
| BRAMY2004387 | 0 | 0 | 0 | 0 | 0 | 30.516 | 0 |
| BRAMY2004492 | 0 | 0 | 0 | 0 | 0 | 100 | 0 |
| BRAMY2004521 | 0 | 0 | 0 | 1.682 | 0 | 7.165 | 0 |
| BRAMY2004524 | 0 | 0 | 0 | 0 | 0 | 21.841 | 0 |
| BRAMY2004537 | 0 | 0 | 0 | 0 | 0 | 100 | 0 |
| BRAMY2004542 | 0 | 0 | 0 | 0 | 0 | 100 | 0 |
| BRAMY2004756 | 0 | 0 | 0 | 0 | 0 | 100 | 0 |
| BRAMY2004771 | 0 | 0 | 0 | 0 | 0 | 100 | 0 |
| BRAMY2005052 | 0 | 0 | 0 | 8.242 | 0 | 46.801 | 0 |
| BRAMY2005064 | 0 | 0 | 0 | 0 | 0 | 100 | 0 |
| BRAMY2005094 | 0 | 0 | 0 | 0 | 0 | 100 | 0 |
| BRAMY2005151 | 0 | 0 | 0 | 0 | 0 | 100 | 0 |
| BRAMY2005182 | 0 | 0 | 0 | 0 | 0 | 100 | 0 |
| BRAMY2005244 | 0 | 0 | 0 | 0 | 0 | 100 | 0 |
| BRAMY2005391 | 0 | 0 | 0 | 0 | 0 | 100 | 0 |
| BRAMY2005488 | 0 | 0 | 0 | 8.088 | 0 | 22.964 | 0 |
| BRAMY2005576 | 0 | 0 | 0 | 0 | 0 | 21.396 | 0 |
| BRAMY2005648 | 0 | 0 | 0 | 0 | 0 | 100 | 0 |
| BRAMY2005662 | 0 | 0 | 0 | 0 | 0 | 100 | 0 |
| BRAMY2005708 | 0 | 0 | 0 | 0 | 0 | 100 | 0 |
| BRAMY2005711 | 0 | 0 | 0 | 0 | 0 | 100 | 0 |
| BRAMY2006092 | 0 | 0 | 0 | 0 | 0 | 100 | 0 |
| BRAMY2006118 | 0 | 0 | 0 | 0 | 0 | 100 | 0 |
| BRAMY2006149 | 0 | 0 | 0 | 0 | 0 | 100 | 0 |
| BRAMY2006366 | 0 | 0 | 0 | 0 | 0 | 9.282 | 0 |
| BRAMY2006375 | 0 | 0 | 0 | 16.823 | 0 | 23.883 | 0 |
| BRAMY2006397 | 0 | 0 | 0 | 0 | 0 | 2.830 | 0 |
| BRAMY2006411 | 0 | 0 | 0 | 0 | 0 | 100 | 0 |
| BRAMY2006459 | 0 | 0 | 0 | 0 | 0 | 100 | 0 |
| BRAMY2006466 | 0 | 0 | 0 | 0 | 0 | 100 | 0 |
| BRAMY2006659 | 0 | 0 | 0 | 0 | 0 | 100 | 0 |
| BRAMY2006779 | 0 | 0 | 0 | 0 | 0 | 100 | 0 |
| BRAMY2006809 | 0 | 0 | 0 | 0 | 0 | 100 | 0 |
| BRAMY2006879 | 0 | 0 | 0 | 0 | 0 | 100 | 0 |
| BRAMY2007043 | 0 | 0 | 0 | 0 | 0 | 100 | 0 |
| BRAMY2007078 | 0 | 0 | 0 | 0 | 0 | 3.832 | 0 |
| BRAMY2007110 | 0 | 0 | 0 | 0 | 0 | 100 | 0 |
| BRAMY2007185 | 0 | 0 | 0 | 0 | 0 | 100 | 0 |
| BRAMY2007244 | 0 | 0 | 0 | 8.319 | 0 | 23.618 | 0 |
| BRAMY2007249 | 0 | 0 | 0 | 0 | 0 | 100 | 0 |
| BRAMY2007255 | 0 | 0 | 0 | 0 | 0 | 59.700 | 0 |
| BRAMY2007287 | 0 | 0 | 0 | 0 | 0 | 10.796 | 0 |
| BRAMY2007308 | 0 | 0 | 0 | 0 | 0 | 100 | 0 |
| BRAMY2007411 | 0 | 0 | 0 | 0 | 0 | 100 | 0 |
| BRAMY2007458 | 0 | 0 | 0 | 0 | 0 | 100 | 0 |
| BRAMY2007486 | 0 | 0 | 0 | 0 | 0 | 100 | 0 |
| BRAMY2007546 | 0 | 0 | 0 | 0 | 0 | 100 | 0 |
| BRAMY2007567 | 0 | 0 | 0 | 16.522 | 0 | 23.456 | 0 |
| BRAMY2007594 | 0 | 0 | 0 | 0 | 0 | 31.238 | 0 |
| BRAMY2007610 | 0 | 0 | 0 | 0 | 0 | 100 | 0 |
| BRAMY2007613 | 0 | 0 | 0 | 3.803 | 4.840 | 9.448 | 0 |
| BRAMY2007653 | 0 | 0 | 0 | 0 | 0 | 100 | 0 |
| BRAMY2007753 | 0 | 0 | 0 | 0 | 0 | 100 | 0 |
| BRAMY2007859 | 0 | 0 | 0 | 0 | 0 | 100 | 0 |
| BRAMY2008333 | 0 | 0 | 0 | 0 | 0 | 100 | 0 |
| BRAMY2008382 | 0 | 0 | 0 | 0 | 0 | 15.624 | 0 |
| BRAMY2008563 | 0 | 0 | 0 | 0 | 0 | 49.688 | 0 |
| BRAMY2008583 | 0 | 0 | 0 | 0 | 0 | 100 | 0 |
| BRAMY2008977 | 0 | 0 | 0 | 0 | 0 | 5.095 | 0 |
| BRAMY2008979 | 0 | 0 | 0 | 0 | 0 | 100 | 0 |
| BRAMY2009013 | 0 | 0 | 0 | 0 | 0 | 6.137 | 0 |
| BRAMY2009023 | 0 | 0 | 0 | 0 | 0 | 13.358 | 0 |
| BRAMY2009034 | 0 | 0 | 0 | 0 | 0 | 100 | 0 |
| BRAMY2009105 | 0 | 0 | 0 | 0 | 0 | 100 | 0 |
| BRAMY2009123 | 0 | 0 | 0 | 0 | 0 | 45.794 | 0 |
| BRAMY2009192 | 0 | 0 | 0 | 0 | 0 | 100 | 0 |
| BRAMY2009344 | 0 | 0 | 0 | 0 | 0 | 100 | 0 |
| BRAMY2009349 | 0 | 0 | 0 | 0 | 0 | 100 | 0 |
| BRAMY2009394 | 0 | 0 | 0 | 0 | 0 | 100 | 0 |
| BRAMY2009489 | 0 | 0 | 0 | 25.513 | 0 | 36.219 | 0 |
| BRAMY2009508 | 0 | 0 | 0 | 31.931 | 0 | 22.665 | 0 |
| BRAMY2009528 | 0 | 0 | 0 | 0 | 0 | 100 | 0 |
| BRAMY2009557 | 0 | 0 | 0 | 0 | 0 | 49.688 | 0 |
| BRAMY2009612 | 0 | 0 | 0 | 0 | 0 | 13.638 | 0 |
| BRAMY2009693 | 0 | 0 | 0 | 0 | 0 | 100 | 0 |
| BRAMY2009934 | 0 | 0 | 0 | 0 | 0 | 5.956 | 0 |
| BRAMY2010068 | 0 | 0 | 0 | 0 | 0 | 100 | 0 |
| BRAMY2010135 | 0 | 0 | 0 | 0 | 0 | 100 | 0 |
| BRAMY2010145 | 0 | 0 | 0 | 0 | 0 | 100 | 0 |
| BRAMY2010171 | 0 | 0 | 0 | 0 | 0 | 100 | 0 |
| BRAMY2010208 | 0 | 0 | 0 | 0 | 0 | 36.630 | 0 |
| BRAMY2010239 | 0 | 0 | 0 | 0 | 0 | 100 | 0 |
| BRAMY2010272 | 0 | 0 | 0 | 0 | 0 | 50.189 | 0 |
| BRAMY2010290 | 0 | 0 | 0 | 0 | 0 | 100 | 0 |
| BRAMY2010357 | 0 | 0 | 0 | 21.196 | 0 | 30.090 | 0 |
| BRAMY2010366 | 0 | 0 | 0 | 0 | 0 | 100 | 0 |
| BRAMY2010395 | 0 | 0 | 0 | 0 | 0 | 100 | 0 |
| BRAMY2010441 | 0 | 0 | 0 | 0 | 0 | 100 | 0 |
| BRAMY2010442 | 0 | 0 | 0 | 0 | 0 | 47.605 | 0 |
| BRAMY2010464 | 0 | 0 | 0 | 15.639 | 0 | 33.302 | 0 |
| BRAMY2010581 | 0 | 0 | 0 | 0 | 0 | 100 | 0 |
| BRAMY2010798 | 0 | 0 | 0 | 0 | 0 | 100 | 0 |
| BRAMY2010808 | 0 | 0 | 0 | 0 | 0 | 100 | 0 |
| BRAMY2010919 | 0 | 0 | 0 | 0 | 0 | 100 | 0 |
| BRAMY2011064 | 0 | 0 | 0 | 0 | 0 | 100 | 0 |
| BRAMY2011105 | 0 | 0 | 0 | 0 | 0 | 3.052 | 0 |
| BRAMY2011178 | 0 | 0 | 0 | 0 | 0 | 100 | 0 |
| BRAMY2011196 | 0 | 0 | 0 | 0 | 0 | 100 | 0 |
| BRAMY2011216 | 0 | 0 | 0 | 0 | 0 | 100 | 0 |
| BRAMY2011253 | 0 | 0 | 0 | 0 | 0 | 100 | 0 |
| BRAMY2011446 | 0 | 0 | 0 | 25.113 | 0 | 35.650 | 0 |
| BRAMY2011480 | 0 | 0 | 0 | 0 | 0 | 100 | 0 |
| BRAMY2011639 | 0 | 0 | 0 | 0 | 0 | 100 | 0 |
| BRAMY2011679 | 0 | 0 | 0 | 0 | 0 | 100 | 0 |
| BRAMY2011811 | 0 | 0 | 0 | 0 | 0 | 100 | 0 |
| BRAMY2011841 | 0 | 0 | 0 | 0 | 0 | 11.118 | 0 |
| BRAMY2011846 | 0 | 0 | 0 | 0 | 0 | 100 | 0 |
| BRAMY2011849 | 0 | 0 | 0 | 0 | 0 | 49.688 | 0 |
| BRAMY2011872 | 0 | 0 | 0 | 0 | 0 | 100 | 0 |
| BRAMY2011961 | 0 | 0 | 0 | 0 | 0 | 100 | 0 |
| BRAMY2012017 | 0 | 0 | 0 | 0 | 0 | 100 | 0 |
| BRAMY2012091 | 0 | 0 | 0 | 0 | 0 | 5.983 | 0 |
| BRAMY2012119 | 0 | 0 | 0 | 0 | 0 | 100 | 0 |
| BRAMY2012162 | 0 | 0 | 0 | 0 | 0 | 100 | 0 |
| BRAMY2012163 | 0 | 0 | 0 | 0 | 0 | 7.967 | 0 |
| BRAMY2012277 | 0 | 0 | 0 | 0 | 0 | 100 | 0 |
| BRAMY2012340 | 0 | 0 | 0 | 0 | 0 | 49.688 | 0 |
| BRAMY2012426 | 0 | 0 | 47.668 | 0 | 0 | 6.228 | 0 |
| BRAMY2012497 | 0 | 0 | 0 | 0 | 0 | 100 | 0 |
| BRAMY2012517 | 0 | 0 | 0 | 0 | 0 | 100 | 0 |
| BRAMY2012536 | 0 | 0 | 0 | 0.410 | 0 | 2.326 | 0 |
| BRAMY2012555 | 0 | 0 | 0 | 0 | 0 | 100 | 0 |
| BRAMY2012568 | 0 | 0 | 0 | 0 | 0 | 100 | 0 |
| BRAMY2012581 | 0 | 0 | 0 | 0 | 0 | 46.034 | 0 |
| BRAMY2012691 | 0 | 0 | 0 | 0 | 0 | 100 | 0 |
| BRAMY2012721 | 0 | 0 | 0 | 0 | 0 | 100 | 0 |
| BRAMY2012731 | 0 | 0 | 0 | 0 | 0 | 100 | 0 |
| BRAMY2012776 | 0 | 0 | 0 | 0 | 0 | 100 | 0 |
| BRAMY2012880 | 0 | 0 | 0 | 0 | 0 | 100 | 0 |
| BRAMY2012881 | 0 | 0 | 0 | 0 | 0 | 47.605 | 0 |
| BRAMY2013169 | 0 | 0 | 0 | 8.071 | 0 | 11.457 | 0 |
| BRAMY2013200 | 0 | 0 | 0 | 0 | 0 | 100 | 0 |
| BRAMY2013216 | 0 | 0 | 0 | 0 | 0 | 100 | 0 |
| BRAMY2013405 | 0 | 0 | 0 | 0 | 0 | 100 | 0 |
| BRAMY2013414 | 0 | 0 | 0 | 0 | 0 | 100 | 0 |
| BRAMY2013572 | 0 | 0 | 0 | 0 | 0 | 100 | 0 |
| BRAMY2013590 | 0 | 0 | 0 | 0 | 0 | 43.537 | 0 |
| BRAMY2013621 | 0 | 0 | 0 | 0 | 0 | 29.482 | 0 |
| BRAMY2013659 | 0 | 0 | 0 | 0 | 0 | 29.269 | 0 |
| BRAMY2013722 | 0 | 0 | 0 | 0 | 0 | 100 | 0 |
| BRAMY2013736 | 0 | 0 | 0 | 0 | 0 | 47.605 | 0 |
| BRAMY2013756 | 0 | 0 | 0 | 0 | 0 | 19.026 | 0 |
| BRAMY2013944 | 0 | 0 | 0 | 0 | 0 | 100 | 0 |
| BRAMY2013975 | 0 | 0 | 0 | 0 | 0 | 100 | 0 |
| BRAMY2014019 | 0 | 0 | 0 | 0 | 0 | 100 | 0 |
| BRAMY2014205 | 0 | 0 | 0 | 0 | 0 | 28.873 | 0 |
| BRAMY2014387 | 0 | 0 | 0 | 0 | 0 | 100 | 0 |
| BRAMY2014462 | 0 | 0 | 0 | 0 | 0 | 49.688 | 0 |
| BRAMY2014469 | 0 | 0 | 0 | 0 | 0 | 100 | 0 |
| BRAMY2014541 | 0 | 0 | 0 | 0 | 0 | 100 | 0 |
| BRAMY2014754 | 0 | 0 | 0 | 5.965 | 5.061 | 4.234 | 0 |
| BRAMY2014813 | 0 | 0 | 0 | 0 | 0 | 19.441 | 0 |
| BRAMY2015211 | 0 | 0 | 0 | 0 | 0 | 13.088 | 0 |
| BRAMY2015251 | 0 | 0 | 0 | 0 | 0 | 15.426 | 0 |
| BRAMY2015311 | 0 | 0 | 0 | 0 | 0 | 100 | 0 |
| BRAMY2015420 | 0 | 0 | 0 | 15.046 | 0 | 21.359 | 0 |
| BRAMY2015503 | 0 | 0 | 0 | 0 | 0 | 100 | 0 |
| BRAMY2015516 | 0 | 0 | 0 | 0 | 0 | 100 | 0 |
| BRAMY2015550 | 0 | 0 | 0 | 0 | 0 | 34.151 | 0 |
| BRAMY2015551 | 0 | 0 | 0 | 0 | 0 | 100 | 0 |
| BRAMY2015559 | 0 | 0 | 0 | 0 | 0 | 100 | 0 |
| BRAMY2015688 | 0 | 0 | 0 | 0 | 0 | 100 | 0 |
| BRAMY2015705 | 0 | 0 | 0 | 0 | 0 | 29.482 | 0 |
| BRAMY2015782 | 0 | 0 | 0 | 0 | 0 | 7.891 | 0 |
| BRAMY2015833 | 0 | 0 | 0 | 9.815 | 0 | 4.645 | 0 |
| BRAMY2015855 | 0 | 0 | 0 | 41.329 | 0 | 58.671 | 0 |
| BRAMY2015890 | 0 | 0 | 0 | 0 | 0 | 100 | 0 |
| BRAMY2015911 | 0 | 0 | 0 | 0 | 0 | 15.012 | 0 |
| BRAMY2015925 | 0 | 0 | 0 | 0 | 0 | 100 | 0 |
| BRAMY2016002 | 0 | 0 | 0 | 0 | 0 | 100 | 0 |
| BRAMY2016009 | 0 | 0 | 0 | 3.712 | 0 | 10.538 | 0 |
| BRAMY2016020 | 0 | 0 | 0 | 0 | 0 | 100 | 0 |
| BRAMY2016070 | 0 | 0 | 0 | 0 | 0 | 100 | 0 |
| BRAMY2016251 | 0 | 0 | 0 | 5.104 | 25.986 | 43.477 | 0 |
| BRAMY2016325 | 0 | 0 | 0 | 0 | 0 | 3.796 | 0 |
| BRAMY2016327 | 0 | 0 | 0 | 0 | 0 | 100 | 0 |
| BRAMY2016386 | 0 | 0 | 0 | 0 | 0 | 54.718 | 0 |
| BRAMY2016706 | 0 | 0 | 0 | 0 | 0 | 4.994 | 0 |
| BRAMY2016771 | 0 | 0 | 0 | 0 | 0 | 100 | 0 |
| BRAMY2016892 | 0 | 0 | 0 | 10.306 | 0 | 14.630 | 0 |
| BRAMY2016938 | 0 | 0 | 0 | 0 | 0 | 100 | 0 |
| BRAMY2016953 | 0 | 0 | 0 | 0 | 0 | 100 | 0 |
| BRAMY2017014 | 0 | 0 | 0 | 0 | 0 | 100 | 0 |
| BRAMY2017249 | 0 | 0 | 0 | 0 | 0 | 100 | 0 |
| BRAMY2017348 | 0 | 0 | 0 | 0 | 0 | 100 | 0 |
| BRAMY2017450 | 0 | 0 | 0 | 0 | 0 | 100 | 0 |
| BRAMY2017455 | 0 | 0 | 0 | 0 | 0 | 100 | 0 |
| BRAMY2017528 | 0 | 0 | 0 | 0 | 0 | 100 | 0 |
| BRAMY2017864 | 0 | 0 | 0 | 0 | 0 | 13.922 | 0 |
| BRAMY2018027 | 0 | 0 | 0 | 0 | 0 | 100 | 0 |
| BRAMY2018106 | 0 | 0 | 0 | 0 | 0 | 100 | 0 |
| BRAMY2018122 | 0 | 0 | 0 | 0 | 0 | 12.783 | 0 |
| BRAMY2018126 | 0 | 0 | 0 | 0 | 0 | 100 | 0 |
| BRAMY2018272 | 0 | 0 | 0 | 0 | 0 | 100 | 0 |
| BRAMY2018273 | 0 | 0 | 0 | 0 | 0 | 100 | 0 |
| BRAMY2018279 | 0 | 0 | 0 | 0 | 0 | 100 | 0 |
| BRAMY2018295 | 0 | 0 | 0 | 0 | 0 | 18.088 | 0 |
| BRAMY2018308 | 0 | 0 | 0 | 0 | 0 | 100 | 0 |
| BRAMY2018396 | 0 | 0 | 0 | 0 | 0 | 100 | 0 |
| BRAMY2018467 | 0 | 0 | 0 | 0 | 0 | 100 | 0 |
| BRAMY2018635 | 0 | 0 | 0 | 0 | 0 | 27.369 | 0 |
| BRAMY2018785 | 0 | 0 | 0 | 0 | 0 | 27.408 | 0 |
| BRAMY2019055 | 0 | 0 | 0 | 0 | 0 | 100 | 0 |
| BRAMY2019111 | 0 | 0 | 0 | 0 | 0 | 30.516 | 0 |
| BRAMY2019277 | 0 | 0 | 0 | 0 | 0 | 100 | 0 |
| BRAMY2019300 | 0 | 0 | 0 | 0 | 0 | 50.189 | 0 |
| BRAMY2019468 | 0 | 0 | 0 | 13.480 | 0 | 11.482 | 0 |
| BRAMY2019509 | 0 | 0 | 0 | 0 | 0 | 100 | 0 |
| BRAMY2019554 | 0 | 0 | 0 | 1.133 | 0 | 4.020 | 0 |
| BRAMY2019600 | 0 | 0 | 0 | 0 | 0 | 8.135 | 0 |
| BRAMY2019643 | 0 | 0 | 0 | 0 | 0 | 60.652 | 0 |
| BRAMY2019710 | 0 | 0 | 0 | 0 | 0 | 47.605 | 0 |
| BRAMY2019963 | 0 | 0 | 0 | 0 | 0 | 6.861 | 0 |
| BRAMY2019985 | 0 | 0 | 0 | 0 | 0 | 100 | 0 |
| BRAMY2019989 | 0 | 0 | 0 | 12.319 | 0 | 5.829 | 0 |
| BRAMY2020050 | 0 | 0 | 0 | 0 | 0 | 100 | 0 |
| BRAMY2020058 | 0 | 0 | 0 | 19.826 | 0 | 28.146 | 0 |
| BRAMY2020106 | 0 | 0 | 0 | 0 | 0 | 11.264 | 0 |
| BRAMY2020270 | 0 | 0 | 0 | 0 | 0 | 100 | 0 |
| BRAMY2020354 | 0 | 0 | 0 | 0 | 0 | 60.652 | 0 |
| BRAMY2020355 | 0 | 0 | 0 | 0 | 0 | 100 | 0 |
| BRAMY2020412 | 0 | 0 | 0 | 0 | 0 | 10.816 | 0 |
| BRAMY2020427 | 0 | 0 | 0 | 1.261 | 2.140 | 1.790 | 0 |
| BRAMY2020445 | 0 | 0 | 0 | 0 | 0 | 100 | 0 |
| BRAMY2020466 | 0 | 0 | 0 | 0 | 0 | 100 | 0 |
| BRAMY2020520 | 0 | 0 | 0 | 0 | 0 | 100 | 0 |
| BRAMY2020574 | 0 | 0 | 0 | 0 | 0 | 100 | 0 |
| BRAMY2020634 | 0 | 0 | 0 | 0 | 0 | 100 | 0 |
| BRAMY2020713 | 0 | 0 | 0 | 18.320 | 0 | 26.008 | 0 |
| BRAMY2021040 | 0 | 0 | 0 | 0 | 0 | 100 | 0 |
| BRAMY2021063 | 0 | 0 | 0 | 0 | 0 | 100 | 0 |
| BRAMY2021098 | 0 | 0 | 0 | 0 | 0 | 100 | 0 |
| BRAMY2021139 | 0 | 0 | 0 | 0 | 0 | 25.787 | 0 |
| BRAMY2021142 | 0 | 0 | 0 | 38.011 | 0 | 26.981 | 0 |
| BRAMY2021276 | 0 | 0 | 0 | 0 | 0 | 47.605 | 0 |
| BRAMY2021310 | 0 | 0 | 0 | 0 | 0 | 12.518 | 0 |
| BRAMY2021388 | 0 | 0 | 0 | 0 | 0 | 100 | 0 |
| BRAMY2021498 | 0 | 0 | 0 | 0 | 0 | 100 | 0 |
| BRAMY2021523 | 0 | 0 | 0 | 0 | 0 | 21.367 | 0 |
| BRAMY2021710 | 0 | 52.794 | 0 | 0 | 0 | 7.579 | 0 |
| BRAMY2021732 | 0 | 0 | 0 | 0 | 0 | 100 | 0 |
| BRAMY2021746 | 0 | 0 | 0 | 0 | 0 | 100 | 0 |
| BRAMY2021825 | 0 | 0 | 0 | 0 | 0 | 66.389 | 0 |
| BRAMY2021867 | 0 | 0 | 0 | 25.113 | 0 | 35.650 | 0 |
| BRAMY2021962 | 0 | 0 | 0 | 0 | 0 | 100 | 0 |
| BRAMY2021982 | 0 | 0 | 0 | 0 | 0 | 100 | 0 |
| BRAMY2022052 | 0 | 0 | 0 | 0 | 0 | 100 | 0 |
| BRAMY2022160 | 0 | 0 | 0 | 0 | 0 | 38.082 | 0 |
| BRAMY2022168 | 0 | 0 | 0 | 0 | 0 | 13.630 | 0 |
| BRAMY2022263 | 0 | 0 | 0 | 0 | 0 | 100 | 0 |
| BRAMY2022301 | 0 | 0 | 0 | 0 | 0 | 100 | 0 |
| BRAMY2022320 | 0 | 0 | 0 | 0 | 0 | 100 | 0 |
| BRAMY2022383 | 0 | 0 | 0 | 26.047 | 0 | 73.953 | 0 |
| BRAMY2022445 | 0 | 0 | 0 | 0 | 0 | 47.355 | 0 |
| BRAMY2022454 | 0 | 0 | 0 | 0 | 0 | 100 | 0 |
| BRAMY2022493 | 0 | 0 | 0 | 1.883 | 0 | 5.346 | 0 |
| BRAMY2022525 | 0 | 0 | 0 | 15.652 | 0 | 22.220 | 0 |
| BRAMY2022532 | 0 | 0 | 0 | 0 | 0 | 100 | 0 |
| BRAMY2022723 | 0 | 0 | 0 | 25.113 | 0 | 35.650 | 0 |
| BRAMY2022786 | 0 | 0 | 0 | 0.886 | 4.509 | 13.830 | 0 |
| BRAMY2022796 | 0 | 0 | 0 | 0 | 0 | 100 | 0 |
| BRAMY2022929 | 0 | 0 | 0 | 0 | 0 | 100 | 0 |
| BRAMY2022980 | 0 | 0 | 0 | 0 | 0 | 15.383 | 0 |
| BRAMY2022984 | 0 | 0 | 0 | 0 | 0 | 100 | 0 |
| BRAMY2023060 | 0 | 0 | 0 | 0 | 0 | 100 | 0 |
| BRAMY2023110 | 0 | 0 | 0 | 0 | 0 | 100 | 0 |
| BRAMY2023115 | 0 | 0 | 0 | 0 | 0 | 49.688 | 0 |
| BRAMY2023161 | 0 | 0 | 0 | 0 | 0 | 100 | 0 |
| BRAMY2023172 | 0 | 0 | 0 | 0 | 0 | 100 | 0 |
| BRAMY2023238 | 0 | 0 | 0 | 0 | 0 | 100 | 0 |
| BRAMY2023327 | 0 | 0 | 0 | 0 | 0 | 100 | 0 |
| BRAMY2023358 | 0 | 0 | 0 | 0 | 0 | 100 | 0 |
| BRAMY2023406 | 0 | 0 | 0 | 0 | 0 | 6.652 | 0 |
| BRAMY2023482 | 0 | 0 | 0 | 0 | 0 | 100 | 0 |
| BRAMY2023514 | 0 | 0 | 0 | 0 | 0 | 100 | 0 |
| BRAMY2023719 | 0 | 0 | 0 | 0 | 0 | 6.024 | 0 |
| BRAMY2023729 | 0 | 0 | 0 | 0 | 0 | 47.355 | 0 |
| BRAMY2023786 | 0 | 0 | 0 | 0 | 0 | 100 | 0 |
| BRAMY2023852 | 0 | 0 | 0 | 0 | 0 | 100 | 0 |
| BRAMY2023863 | 0 | 0 | 0 | 0 | 0 | 35.669 | 0 |
| BRAMY2023939 | 0 | 0 | 0 | 0 | 0 | 6.159 | 0 |
| BRAMY2024004 | 0 | 0 | 0 | 1.963 | 0 | 2.787 | 0 |
| BRAMY2024073 | 0 | 0 | 0 | 0 | 0 | 100 | 0 |
| BRAMY2024312 | 0 | 0 | 0 | 0 | 0 | 49.688 | 0 |
| BRAMY2024374 | 0 | 0 | 0 | 0 | 0 | 100 | 0 |
| BRAMY2024390 | 0 | 0 | 0 | 0 | 0 | 35.210 | 0 |
| BRAMY2024416 | 0 | 0 | 0 | 3.212 | 0 | 22.796 | 0 |
| BRAMY2024449 | 0 | 0 | 0 | 0 | 0 | 100 | 0 |
| BRAMY2024489 | 0 | 0 | 0 | 0 | 0 | 100 | 0 |
| BRAMY2024497 | 0 | 0 | 0 | 0 | 0 | 100 | 0 |
| BRAMY2024514 | 0 | 0 | 0 | 0 | 0 | 100 | 0 |
| BRAMY2024530 | 0 | 0 | 0 | 0 | 0 | 100 | 0 |
| BRAMY2024535 | 0 | 0 | 0 | 0 | 0 | 100 | 0 |
| BRAMY2024545 | 0 | 0 | 0 | 0 | 0 | 100 | 0 |
| BRAMY2024576 | 0 | 0 | 0 | 0 | 0 | 100 | 0 |
| BRAMY2024593 | 0 | 0 | 0 | 0 | 0 | 25.978 | 0 |
| BRAMY2024711 | 0 | 0 | 0 | 0 | 0 | 12.354 | 0 |
| BRAMY2024715 | 0 | 0 | 0 | 0 | 0 | 12.836 | 0 |
| BRAMY2024728 | 0 | 0 | 0 | 0 | 0 | 100 | 0 |
| BRAMY2024827 | 0 | 0 | 0 | 0 | 0 | 4.020 | 0 |
| BRAMY2024849 | 0 | 0 | 0 | 0 | 0 | 100 | 0 |
| BRAMY2025024 | 0 | 0 | 0 | 0 | 0 | 100 | 0 |
| BRAMY2025032 | 0 | 0 | 0 | 0 | 0 | 100 | 0 |
| BRAMY2025121 | 0 | 0 | 0 | 11.752 | 0 | 33.367 | 0 |
| BRAMY2025175 | 0 | 0 | 0 | 0 | 0 | 100 | 0 |
| BRAMY2025218 | 0 | 0 | 0 | 0 | 0 | 100 | 0 |
| BRAMY2025230 | 0 | 0 | 0 | 6.959 | 0 | 19.758 | 0 |
| BRAMY2025272 | 0 | 0 | 0 | 0 | 0 | 47.605 | 0 |
| BRAMY2025277 | 0 | 0 | 0 | 0 | 0 | 100 | 0 |
| BRAMY2025495 | 0 | 0 | 0 | 0 | 0 | 100 | 0 |
| BRAMY2025812 | 0 | 0 | 0 | 0 | 0 | 100 | 0 |
| BRAMY2025996 | 0 | 0 | 0 | 0 | 0 | 100 | 0 |
| BRAMY2026009 | 0 | 0 | 0 | 0 | 0 | 44.952 | 0 |
| BRAMY2026091 | 0 | 0 | 0 | 0 | 0 | 100 | 0 |
| BRAMY2026168 | 0 | 0 | 0 | 0 | 0 | 32.330 | 0 |
| BRAMY2026300 | 0 | 0 | 0 | 0 | 0 | 100 | 0 |
| BRAMY2026347 | 0 | 0 | 0 | 0 | 0 | 100 | 0 |
| BRAMY2026395 | 0 | 0 | 0 | 0 | 0 | 100 | 0 |
| BRAMY2026405 | 0 | 0 | 0 | 0 | 0 | 100 | 0 |
| BRAMY2026533 | 0 | 0 | 0 | 0 | 0 | 18.861 | 0 |
| BRAMY2026538 | 0 | 0 | 0 | 0 | 0 | 100 | 0 |
| BRAMY2026685 | 0 | 34.791 | 0 | 0 | 0 | 2.497 | 0 |
| BRAMY2026713 | 0 | 0 | 0 | 0 | 0 | 100 | 0 |
| BRAMY2026778 | 0 | 34.006 | 0 | 0 | 0 | 4.882 | 0 |
| BRAMY2026824 | 0 | 0 | 0 | 0 | 0 | 100 | 0 |
| BRAMY2026899 | 0 | 0 | 0 | 0 | 0 | 100 | 0 |
| BRAMY2026902 | 0 | 0 | 0 | 0 | 0 | 100 | 0 |
| BRAMY2026904 | 0 | 0 | 0 | 0 | 0 | 100 | 0 |
| BRAMY2026976 | 0 | 0 | 0 | 0 | 0 | 100 | 0 |
| BRAMY2027073 | 0 | 0 | 0 | 0 | 0 | 100 | 0 |
| BRAMY2027114 | 0 | 0 | 0 | 0 | 0 | 17.318 | 0 |
| BRAMY2027140 | 0 | 0 | 0 | 0 | 0 | 51.968 | 0 |
| BRAMY2027242 | 0 | 0 | 0 | 0 | 0 | 100 | 0 |
| BRAMY2027396 | 0 | 0 | 0 | 0 | 0 | 100 | 0 |
| BRAMY2027451 | 0 | 0 | 0 | 0 | 0 | 100 | 0 |
| BRAMY2027461 | 0 | 0 | 0 | 0 | 0 | 100 | 0 |
| BRAMY2027467 | 0 | 0 | 0 | 0 | 0 | 100 | 0 |
| BRAMY2027587 | 0 | 0 | 0 | 9.477 | 0 | 26.909 | 0 |
| BRAMY2027714 | 0 | 0 | 0 | 0 | 0 | 100 | 0 |
| BRAMY2027717 | 0 | 0 | 0 | 0 | 0 | 16.593 | 0 |
| BRAMY2027739 | 0 | 0 | 0 | 0 | 0 | 1.356 | 0 |
| BRAMY2027752 | 0 | 0 | 0 | 0 | 0 | 100 | 0 |
| BRAMY2027782 | 0 | 0 | 0 | 0 | 0 | 100 | 0 |
| BRAMY2028072 | 0 | 0 | 0 | 0 | 0 | 100 | 0 |
| BRAMY2028183 | 0 | 0 | 0 | 13.334 | 0 | 18.929 | 0 |
| BRAMY2028188 | 0 | 0 | 0 | 0 | 0 | 100 | 0 |
| BRAMY2028269 | 0 | 0 | 0 | 0 | 0 | 32.892 | 0 |
| BRAMY2028282 | 0 | 0 | 0 | 0 | 0 | 24.592 | 0 |
| BRAMY2028472 | 0 | 0 | 0 | 0 | 0 | 100 | 0 |
| BRAMY2028491 | 0 | 0 | 0 | 0 | 0 | 36.150 | 0 |
| BRAMY2028516 | 0 | 0 | 0 | 0 | 0 | 3.974 | 0 |
| BRAMY2028565 | 0 | 0 | 0 | 0 | 0 | 100 | 0 |
| BRAMY2028593 | 0 | 0 | 0 | 0 | 0 | 100 | 0 |
| BRAMY2028682 | 0 | 0 | 0 | 0 | 0 | 21.396 | 0 |
| BRAMY2028740 | 0 | 0 | 0 | 1.322 | 10.092 | 2.814 | 0 |
| BRAMY2028783 | 0 | 0 | 0 | 0 | 0 | 100 | 0 |
| BRAMY2028796 | 0 | 10.433 | 0 | 0 | 0 | 4.493 | 0 |
| BRAMY2028856 | 0 | 0 | 0 | 0 | 0 | 100 | 0 |
| BRAMY2028914 | 0 | 0 | 0 | 0 | 0 | 17.849 | 0 |
| BRAMY2029204 | 0 | 0 | 0 | 0 | 0 | 100 | 0 |
| BRAMY2029602 | 0 | 0 | 0 | 0 | 0 | 100 | 0 |
| BRAMY2029988 | 0 | 0 | 0 | 8.360 | 0 | 23.737 | 0 |
| BRAMY2030098 | 0 | 0 | 0 | 29.935 | 0 | 42.496 | 0 |
| BRAMY2030109 | 0 | 0 | 0 | 0 | 0 | 100 | 0 |
| BRAMY2030206 | 0 | 0 | 0 | 0 | 0 | 100 | 0 |
| BRAMY2030477 | 0 | 0 | 0 | 0 | 0 | 100 | 0 |
| BRAMY2030545 | 0 | 0 | 0 | 0 | 0 | 22.530 | 0 |
| BRAMY2030607 | 0 | 0 | 0 | 0 | 0 | 100 | 0 |
| BRAMY2030702 | 0 | 0 | 0 | 0 | 0 | 100 | 0 |
| BRAMY2030703 | 0 | 0 | 0 | 0 | 0 | 100 | 0 |
| BRAMY2030768 | 0 | 0 | 0 | 0 | 0 | 100 | 0 |
| BRAMY2030799 | 0 | 0 | 0 | 0 | 0 | 100 | 0 |
| BRAMY2030859 | 0 | 0 | 0 | 0 | 0 | 100 | 0 |
| BRAMY2030915 | 0 | 0 | 0 | 0 | 0 | 100 | 0 |
| BRAMY2031317 | 0 | 0 | 0 | 33.400 | 0 | 15.805 | 0 |
| BRAMY2031377 | 0 | 0 | 0 | 0 | 0 | 100 | 0 |
| BRAMY2031442 | 0 | 0 | 0 | 0 | 0 | 100 | 0 |
| BRAMY2031516 | 0 | 0 | 0 | 0 | 0 | 7.442 | 0 |
| BRAMY2032014 | 0 | 0 | 0 | 0 | 0 | 100 | 0 |
| BRAMY2032087 | 0 | 17.000 | 0 | 0 | 0 | 2.440 | 0 |
| BRAMY2032231 | 0 | 0 | 0 | 0 | 0 | 100 | 0 |
| BRAMY2032242 | 0 | 0 | 0 | 0 | 0 | 100 | 0 |
| BRAMY2032290 | 0 | 0 | 0 | 0 | 0 | 100 | 0 |
| BRAMY2032311 | 0 | 0 | 0 | 0 | 0 | 100 | 0 |
| BRAMY2032317 | 0 | 0 | 0 | 0 | 0 | 60.652 | 0 |
| BRAMY2032344 | 0 | 0 | 0 | 0 | 0 | 100 | 0 |
| BRAMY2032589 | 0 | 0 | 0 | 0 | 0 | 100 | 0 |
| BRAMY2032678 | 0 | 0 | 0 | 0 | 0 | 100 | 0 |
| BRAMY2032696 | 0 | 0 | 0 | 0 | 0 | 21.396 | 0 |
| BRAMY2032777 | 0 | 0 | 0 | 0 | 0 | 100 | 0 |
| BRAMY2032994 | 0 | 0 | 0 | 0 | 0 | 100 | 0 |
| BRAMY2033003 | 0 | 0 | 0 | 0 | 0 | 100 | 0 |
| BRAMY2033116 | 0 | 0 | 0 | 0 | 0 | 100 | 0 |
| BRAMY2033240 | 0 | 0 | 0 | 0 | 0 | 100 | 0 |
| BRAMY2033267 | 0 | 0 | 0 | 0 | 0 | 100 | 0 |
| BRAMY2033316 | 0 | 0 | 0 | 0 | 0 | 54.718 | 0 |
| BRAMY2033332 | 0 | 0 | 0 | 0 | 0 | 100 | 0 |
| BRAMY2033506 | 0 | 0 | 0 | 0 | 0 | 100 | 0 |
| BRAMY2033594 | 0 | 0 | 0 | 0 | 0 | 100 | 0 |
| BRAMY2033895 | 0 | 0 | 0 | 0 | 0 | 100 | 0 |
| BRAMY2033914 | 0 | 0 | 0 | 0 | 0 | 50.189 | 0 |
| BRAMY2034072 | 0 | 0 | 0 | 0 | 0 | 100 | 0 |
| BRAMY2034185 | 0 | 0 | 0 | 0 | 0 | 100 | 0 |
| BRAMY2034187 | 0 | 0 | 0 | 0 | 0 | 12.529 | 0 |
| BRAMY2034305 | 0 | 0 | 0 | 0 | 0 | 100 | 0 |
| BRAMY2034561 | 0 | 0 | 0 | 0 | 0 | 100 | 0 |
| BRAMY2034920 | 0 | 0 | 0 | 0 | 0 | 100 | 0 |
| BRAMY2034993 | 0 | 0 | 0 | 0 | 0 | 100 | 0 |
| BRAMY2035070 | 0 | 0 | 0 | 0 | 0 | 54.718 | 0 |
| BRAMY2035449 | 0 | 0 | 0 | 0 | 0 | 100 | 0 |
| BRAMY2035545 | 0 | 0 | 0 | 41.329 | 0 | 58.671 | 0 |
| BRAMY2035606 | 0 | 0 | 0 | 0 | 0 | 100 | 0 |
| BRAMY2035718 | 0 | 0 | 0 | 0 | 0 | 100 | 0 |
| BRAMY2035719 | 0 | 0 | 0 | 0 | 0 | 15.304 | 0 |
| BRAMY2035801 | 0 | 0 | 0 | 0 | 0 | 100 | 0 |
| BRAMY2035869 | 0 | 0 | 0 | 0 | 0 | 60.652 | 0 |
| BRAMY2035923 | 0 | 0 | 0 | 0 | 0 | 100 | 0 |
| BRAMY2036076 | 0 | 0 | 0 | 15.354 | 0 | 21.797 | 0 |
| BRAMY2036079 | 0 | 0 | 0 | 0 | 0 | 100 | 0 |
| BRAMY2036254 | 0 | 0 | 0 | 0 | 0 | 100 | 0 |
| BRAMY2036266 | 0 | 0 | 0 | 0 | 0 | 100 | 0 |
| BRAMY2036387 | 0 | 0 | 0 | 0 | 0 | 13.490 | 0 |
| BRAMY2036396 | 0 | 0 | 0 | 0 | 0 | 100 | 0 |
| BRAMY2036459 | 0 | 0 | 0 | 0 | 0 | 3.798 | 0 |
| BRAMY2036567 | 0 | 0 | 0 | 26.525 | 0 | 22.594 | 0 |
| BRAMY2036615 | 0 | 0 | 0 | 0 | 0 | 100 | 0 |
| BRAMY2036699 | 0 | 0 | 0 | 0 | 0 | 100 | 0 |
| BRAMY2036913 | 0 | 0 | 0 | 0 | 0 | 100 | 0 |
| BRAMY2036918 | 0 | 0 | 0 | 0 | 0 | 100 | 0 |
| BRAMY2036968 | 0 | 0 | 0 | 0 | 0 | 100 | 0 |
| BRAMY2037147 | 0 | 0 | 0 | 0 | 0 | 36.150 | 0 |
| BRAMY2037328 | 0 | 0 | 0 | 0 | 0 | 100 | 0 |
| BRAMY2037609 | 0 | 0 | 0 | 0 | 0 | 100 | 0 |
| BRAMY2037629 | 0 | 0 | 0 | 0 | 0 | 100 | 0 |
| BRAMY2037800 | 0 | 0 | 0 | 0 | 0 | 100 | 0 |
| BRAMY2037823 | 0 | 0 | 0 | 21.055 | 0 | 29.890 | 0 |
| BRAMY2037971 | 0 | 0 | 0 | 0 | 0 | 100 | 0 |
| BRAMY2038100 | 0 | 0 | 0 | 0 | 0 | 100 | 0 |
| BRAMY2038128 | 0 | 0 | 0 | 0 | 0 | 100 | 0 |
| BRAMY2038317 | 0 | 0 | 0 | 0 | 0 | 100 | 0 |
| BRAMY2038475 | 0 | 0 | 0 | 0 | 0 | 100 | 0 |
| BRAMY2038484 | 0 | 0 | 0 | 0 | 0 | 100 | 0 |
| BRAMY2038516 | 0 | 0 | 0 | 0 | 0 | 12.770 | 0 |
| BRAMY2038832 | 0 | 0 | 0 | 0 | 0 | 12.282 | 0 |
| BRAMY2038846 | 0 | 0 | 0 | 0 | 0 | 47.355 | 0 |
| BRAMY2038887 | 0 | 0 | 0 | 0 | 0 | 100 | 0 |
| BRAMY2038904 | 0 | 0 | 0 | 0 | 0 | 60.652 | 0 |
| BRAMY2039287 | 0 | 0 | 0 | 0 | 0 | 100 | 0 |
| BRAMY2039300 | 0 | 0 | 0 | 0 | 0 | 100 | 0 |
| BRAMY2039314 | 0 | 0 | 0 | 0 | 0 | 100 | 0 |
| BRAMY2039341 | 0 | 0 | 0 | 0 | 0 | 100 | 0 |
| BRAMY2039630 | 0 | 0 | 0 | 0 | 0 | 8.744 | 0 |
| BRAMY2039695 | 0 | 0 | 0 | 0 | 0 | 100 | 0 |
| BRAMY2039728 | 0 | 0 | 0 | 25.927 | 0 | 36.806 | 0 |
| BRAMY2039872 | 0 | 0 | 0 | 14.071 | 0 | 19.976 | 0 |
| BRAMY2040051 | 0 | 0 | 0 | 0 | 0 | 100 | 0 |
| BRAMY2040095 | 0 | 0 | 10.544 | 3.154 | 0 | 2.755 | 0 |
| BRAMY2040159 | 0 | 0 | 0 | 0 | 0 | 100 | 0 |
| BRAMY2040214 | 0 | 0 | 0 | 10.294 | 0 | 29.228 | 0 |
| BRAMY2040478 | 0 | 0 | 0 | 0 | 0 | 100 | 0 |
| BRAMY2040592 | 0 | 0 | 0 | 0 | 12.582 | 7.017 | 0 |
| BRAMY2040915 | 0 | 0 | 0 | 0 | 0 | 100 | 0 |
| BRAMY2041261 | 0 | 0 | 0 | 0 | 0 | 100 | 0 |
| BRAMY2041347 | 0 | 0 | 0 | 0 | 0 | 100 | 0 |
| BRAMY2041378 | 0 | 0 | 0 | 0 | 0 | 100 | 0 |
| BRAMY2041384 | 0 | 0 | 0 | 0 | 0 | 100 | 0 |
| BRAMY2041434 | 0 | 0 | 0 | 0 | 0 | 100 | 0 |
| BRAMY2041507 | 0 | 0 | 0 | 0 | 0 | 100 | 0 |
| BRAMY2041542 | 0 | 0 | 0 | 0 | 0 | 100 | 0 |
| BRAMY2041738 | 0 | 0 | 0 | 0 | 0 | 100 | 0 |
| BRAMY2042122 | 0 | 0 | 0 | 0 | 0 | 100 | 0 |
| BRAMY2042131 | 0 | 0 | 0 | 0 | 0 | 100 | 0 |
| BRAMY2042178 | 0 | 0 | 0 | 0 | 0 | 100 | 0 |
| BRAMY2042195 | 0 | 0 | 0 | 0 | 0 | 30.516 | 0 |
| BRAMY2042244 | 0 | 0 | 0 | 0 | 0 | 100 | 0 |
| BRAMY2042549 | 0 | 0 | 0 | 0 | 0 | 100 | 0 |
| BRAMY2042596 | 0 | 0 | 0 | 0 | 0 | 100 | 0 |
| BRAMY2042612 | 0 | 0 | 0 | 0 | 0 | 100 | 0 |
| BRAMY2042641 | 0 | 0 | 0 | 0 | 0 | 100 | 0 |
| BRAMY2042711 | 0 | 0 | 0 | 0 | 0 | 100 | 0 |
| BRAMY2042760 | 0 | 0 | 0 | 0 | 0 | 100 | 0 |
| BRAMY2042804 | 0 | 0 | 0 | 41.329 | 0 | 58.671 | 0 |
| BRAMY2042899 | 0 | 0 | 0 | 0 | 0 | 100 | 0 |
| BRAMY2042918 | 0 | 0 | 0 | 0 | 0 | 100 | 0 |
| BRAMY2043069 | 0 | 0 | 0 | 0 | 0 | 21.396 | 0 |
| BRAMY2043103 | 0 | 0 | 0 | 0 | 0 | 100 | 0 |
| BRAMY2043314 | 0 | 0 | 0 | 0 | 0 | 100 | 0 |
| BRAMY2043345 | 0 | 0 | 0 | 0 | 0 | 100 | 0 |
| BRAMY2044246 | 0 | 0 | 0 | 0 | 0 | 100 | 0 |
| BRAMY2044317 | 0 | 0 | 0 | 0 | 0 | 100 | 0 |
| BRAMY2044349 | 0 | 0 | 0 | 0 | 0 | 100 | 0 |
| BRAMY2044686 | 0 | 0 | 0 | 0 | 0 | 100 | 0 |
| BRAMY2044838 | 0 | 0 | 0 | 0 | 0 | 31.238 | 0 |
| BRAMY2045036 | 0 | 0 | 0 | 0 | 0 | 100 | 0 |
| BRAMY2045299 | 0 | 0 | 0 | 0 | 0 | 100 | 0 |
| BRAMY2045471 | 0 | 0 | 0 | 0 | 0 | 100 | 0 |
| BRAMY2045530 | 0 | 66.216 | 0 | 0 | 0 | 9.506 | 0 |
| BRAMY2046109 | 0 | 0 | 0 | 0 | 0 | 100 | 0 |
| BRAMY2046478 | 0 | 0 | 0 | 0 | 0 | 100 | 0 |
| BRAMY2046489 | 0 | 0 | 0 | 0 | 0 | 100 | 0 |
| BRAMY2046537 | 0 | 0 | 0 | 0 | 0 | 100 | 0 |
| BRAMY2046667 | 0 | 0 | 0 | 0 | 0 | 100 | 0 |
| BRAMY2046742 | 0 | 0 | 0 | 0 | 0 | 100 | 0 |
| BRAMY2046871 | 0 | 0 | 0 | 0 | 0 | 66.835 | 0 |
| BRAMY2046984 | 0 | 0 | 0 | 0 | 0 | 100 | 0 |
| BRAMY2046989 | 0 | 0 | 0 | 0 | 0 | 100 | 0 |
| BRAMY2047169 | 0 | 0 | 0 | 0 | 0 | 100 | 0 |
| BRAMY2047280 | 0 | 0 | 0 | 0 | 0 | 100 | 0 |
| BRAMY2047420 | 0 | 0 | 0 | 0.719 | 0 | 2.041 | 0 |
| BRAMY2047676 | 0 | 0 | 0 | 0 | 0 | 100 | 0 |
| BRAMY2047692 | 0 | 0 | 0 | 0 | 0 | 100 | 0 |
| BRAMY2047746 | 0 | 0 | 0 | 0 | 0 | 100 | 0 |
| BRAMY2047751 | 0 | 0 | 0 | 0 | 0 | 100 | 0 |
| BRAMY2047765 | 0 | 0 | 0 | 0 | 0 | 100 | 0 |
| BRAMY2047884 | 0 | 0 | 0 | 0 | 0 | 100 | 0 |
| BRAMY2047948 | 0 | 0 | 0 | 0 | 0 | 100 | 0 |
| BRAMY3000206 | 0 | 0 | 0 | 0 | 0 | 100 | 0 |
| BRAMY3000210 | 0 | 0 | 0 | 0 | 0 | 100 | 0 |
| BRAMY3000213 | 0 | 0 | 0 | 0 | 0 | 100 | 0 |
| BRAMY3000224 | 0 | 0 | 0 | 0 | 0 | 28.785 | 0 |
| BRAMY3000254 | 0 | 0 | 0 | 0 | 0 | 100 | 0 |
| BRAMY3000307 | 0 | 0 | 0 | 0 | 0 | 100 | 0 |
| BRAMY3000692 | 0 | 0 | 0 | 0 | 0 | 100 | 0 |
| BRAMY3001356 | 0 | 0 | 0 | 0 | 0 | 100 | 0 |
| BRAMY3001401 | 0 | 0 | 0 | 0 | 0 | 100 | 0 |
| BRAMY3001409 | 0 | 0 | 0 | 0 | 0 | 100 | 0 |
| BRAMY3001794 | 0 | 0 | 0 | 0 | 0 | 100 | 0 |
| BRAMY3002120 | 0 | 0 | 0 | 0 | 0 | 54.718 | 0 |
| BRAMY3002312 | 0 | 0 | 0 | 0 | 0 | 100 | 0 |
| BRAMY3002329 | 0 | 0 | 0 | 9.616 | 0 | 20.478 | 0 |
| BRAMY3002458 | 0 | 0 | 0 | 0 | 0 | 12.509 | 0 |
| BRAMY3002508 | 0 | 0 | 0 | 0 | 0 | 100 | 0 |
| BRAMY3002620 | 0 | 0 | 0 | 41.329 | 0 | 58.671 | 0 |
| BRAMY3002803 | 0 | 0 | 0 | 0 | 0 | 14.538 | 0 |
| BRAMY3002805 | 0 | 0 | 0 | 0 | 0 | 100 | 0 |
| BRAMY3002886 | 0 | 0 | 0 | 0 | 0 | 100 | 0 |
| BRAMY3002991 | 0 | 0 | 0 | 0 | 0 | 3.951 | 0 |
| BRAMY3003026 | 0 | 0 | 0 | 0 | 0 | 44.784 | 0 |
| BRAMY3003109 | 0 | 0 | 0 | 0 | 0 | 100 | 0 |
| BRAMY3003205 | 0 | 0 | 0 | 0 | 0 | 48.484 | 0 |
| BRAMY3003310 | 0 | 0 | 0 | 0 | 0 | 54.718 | 0 |
| BRAMY3003566 | 0 | 0 | 0 | 0 | 0 | 100 | 0 |
| BRAMY3003723 | 0 | 0 | 0 | 0 | 0 | 100 | 0 |
| BRAMY3004126 | 0 | 0 | 0 | 0 | 0 | 50.189 | 0 |
| BRAMY3004224 | 0 | 0 | 0 | 0 | 0 | 33.278 | 0 |
| BRAMY3004364 | 0 | 0 | 0 | 4.821 | 0 | 13.689 | 0 |
| BRAMY3004377 | 0 | 0 | 0 | 1.766 | 0 | 3.343 | 0 |
| BRAMY3004672 | 0 | 0 | 0 | 0 | 0 | 100 | 0 |
| BRAMY3004800 | 0 | 0 | 0 | 0 | 0 | 15.340 | 0 |
| BRAMY3004900 | 0 | 0 | 0 | 0 | 0 | 100 | 0 |
| BRAMY3004919 | 0 | 0 | 0 | 0 | 0 | 100 | 0 |
| BRAMY3005091 | 0 | 0 | 0 | 13.622 | 0 | 19.339 | 0 |
| BRAMY3005184 | 0 | 0 | 0 | 0 | 0 | 22.578 | 0 |
| BRAMY3005656 | 0 | 0 | 0 | 0 | 0 | 100 | 0 |
| BRAMY3005912 | 0 | 0 | 0 | 30.401 | 0 | 21.579 | 0 |
| BRAMY3005932 | 0 | 0 | 0 | 41.329 | 0 | 58.671 | 0 |
| BRAMY3006032 | 0 | 0 | 0 | 0 | 0 | 100 | 0 |
| BRAMY3006297 | 0 | 0 | 0 | 0 | 0 | 100 | 0 |
| BRAMY3006761 | 0 | 0 | 0 | 0 | 0 | 11.062 | 0 |
| BRAMY3007078 | 0 | 0 | 0 | 0 | 0 | 49.688 | 0 |
| BRAMY3007206 | 0 | 0 | 0 | 0 | 0 | 100 | 0 |
| BRAMY3007311 | 0 | 0 | 0 | 5.576 | 0 | 23.748 | 0 |
| BRAMY3007350 | 0 | 0 | 0 | 0 | 0 | 100 | 0 |
| BRAMY3007449 | 0 | 0 | 0 | 0 | 0 | 100 | 0 |
| BRAMY3007471 | 0 | 0 | 0 | 0 | 0 | 100 | 0 |
| BRAMY3007609 | 0 | 0 | 0 | 0 | 0 | 100 | 0 |
| BRAMY3007723 | 0 | 0 | 0 | 0 | 0 | 100 | 0 |
| BRAMY3007968 | 0 | 0 | 0 | 0 | 0 | 100 | 0 |
| BRAMY3008044 | 0 | 0 | 0 | 0 | 0 | 16.267 | 0 |
| BRAMY3008088 | 0 | 0 | 0 | 0 | 0 | 100 | 0 |
| BRAMY3008096 | 0 | 0 | 0 | 41.329 | 0 | 58.671 | 0 |
| BRAMY3008335 | 0 | 0 | 0 | 37.822 | 0 | 17.898 | 0 |
| BRAMY3008466 | 0 | 0 | 0 | 0 | 0 | 100 | 0 |
| BRAMY3008505 | 0 | 0 | 0 | 0 | 0 | 100 | 0 |
| BRAMY3008650 | 0 | 0 | 0 | 0 | 0 | 100 | 0 |
| BRAMY3008937 | 0 | 0 | 0 | 0 | 0 | 100 | 0 |
| BRAMY3009158 | 0 | 0 | 0 | 0 | 0 | 100 | 0 |
| BRAMY3009207 | 0 | 0 | 0 | 0 | 0 | 100 | 0 |
| BRAMY3009491 | 0 | 0 | 0 | 22.146 | 0 | 31.439 | 0 |
| BRAMY3009556 | 0 | 0 | 0 | 0 | 0 | 47.605 | 0 |
| BRAMY3009782 | 0 | 0 | 0 | 0 | 0 | 47.605 | 0 |
| BRAMY3009811 | 0 | 0 | 0 | 0 | 0 | 33.057 | 0 |
| BRAMY3009904 | 0 | 0 | 0 | 0 | 0 | 100 | 0 |
| BRAMY3010008 | 0 | 0 | 0 | 0 | 0 | 1.173 | 0 |
| BRAMY3010264 | 0 | 0 | 0 | 0 | 0 | 50.189 | 0 |
| BRAMY3010321 | 0 | 0 | 0 | 0 | 0 | 100 | 0 |
| BRAMY3010411 | 0 | 0 | 0 | 0 | 0 | 100 | 0 |
| BRAMY3010492 | 0 | 0 | 0 | 0 | 0 | 5.803 | 0 |
| BRAMY3010603 | 0 | 0 | 0 | 0 | 0 | 100 | 0 |
| BRAMY3010654 | 0 | 0 | 0 | 0 | 0 | 47.605 | 0 |
| BRAMY4000089 | 0 | 0 | 0 | 3.612 | 0 | 10.255 | 0 |
| BRAMY4000095 | 0 | 0 | 0 | 0 | 0 | 14.721 | 0 |
| BRAMY4000229 | 0 | 0 | 0 | 25.113 | 0 | 35.650 | 0 |
| BRAMY4000277 | 0 | 0 | 0 | 0 | 0 | 100 | 0 |
| BRAMY4000915 | 0 | 0 | 0 | 0 | 0 | 49.688 | 0 |
| BRAMY4000962 | 0 | 0 | 0 | 0 | 0 | 100 | 0 |
| BRAMY4001173 | 0 | 0 | 0 | 0 | 0 | 100 | 0 |
| BRAMY4001200 | 0 | 0 | 0 | 0 | 0 | 100 | 0 |
| BRAMY4001234 | 0 | 0 | 0 | 0 | 0 | 54.718 | 0 |
| BRAMY4001449 | 0 | 0 | 0 | 0 | 0 | 100 | 0 |
| BRAMY4001652 | 0 | 0 | 0 | 0 | 0 | 100 | 0 |
| BRAMY4001863 | 0 | 0 | 0 | 0 | 0 | 15.094 | 0 |
| BRAMY4001913 | 0 | 0 | 0 | 0 | 0 | 100 | 0 |
| BRAMY4002575 | 0 | 0 | 0 | 0 | 0 | 100 | 0 |
| BRAMY4002628 | 0 | 0 | 0 | 0 | 0 | 7.157 | 0 |
| BRASW1000053 | 0 | 0 | 0 | 0 | 0 | 0 | 100 |
| BRASW1000125 | 0 | 0 | 0 | 0 | 0 | 0 | 99.054 |
| BRAWH1000001 | 0 | 0 | 0 | 4.724 | 12.024 | 0 | 0 |
| BRAWH1000037 | 0 | 0 | 0 | 2.588 | 0 | 1.837 | 0 |
| BRAWH1000040 | 0 | 0 | 0 | 0 | 0 | 2.773 | 0 |
| BRAWH1000045 | 0 | 0 | 0 | 0.710 | 0 | 16.138 | 0 |
| BRAWH1000083 | 0 | 31.801 | 0 | 3.216 | 0 | 4.565 | 0 |
| BRAWH1000093 | 0 | 0 | 0 | 0.655 | 0 | 0 | 0 |
| BRAWH1000094 | 0 | 0 | 0 | 11.953 | 0 | 4.242 | 0 |
| BRAWH1000127 | 0 | 0 | 0 | 4.538 | 0 | 9.663 | 0 |
| BRAWH1000162 | 0 | 0 | 0 | 5.181 | 6.594 | 9.194 | 0 |
| BRAWH1000174 | 0 | 0 | 0 | 0 | 0 | 8.660 | 0 |
| BRAWH2000082 | 0 | 0 | 0 | 1.634 | 5.544 | 12.368 | 0 |
| BRAWH2000131 | 0 | 0 | 0 | 0 | 78.324 | 0 | 0 |
| BRAWH2000177 | 0 | 0 | 0 | 11.350 | 0 | 0 | 0 |
| BRAWH2000274 | 0 | 0 | 0 | 30.824 | 0 | 9.945 | 0 |
| BRAWH2000353 | 0 | 0 | 0 | 41.512 | 0 | 0 | 0 |
| BRAWH2000417 | 0 | 0 | 0 | 0 | 20.093 | 28.015 | 0 |
| BRAWH2000443 | 0 | 0 | 0 | 0 | 0 | 22.835 | 0 |
| BRAWH2000460 | 0 | 0 | 0 | 9.097 | 0 | 1.230 | 0 |
| BRAWH2000482 | 0 | 0 | 0 | 0 | 0 | 4.588 | 0 |
| BRAWH2000494 | 0 | 0 | 0 | 6.452 | 0 | 0 | 0 |
| BRAWH2000500 | 0 | 0 | 0 | 41.512 | 0 | 0 | 0 |
| BRAWH2000522 | 0 | 0 | 0 | 0 | 6.549 | 3.653 | 0 |
| BRAWH2000595 | 0 | 87.530 | 0 | 0 | 0 | 0 | 0 |
| BRAWH2000633 | 41.554 | 0 | 10.223 | 0 | 0 | 1.336 | 0 |
| BRAWH2000636 | 0 | 0 | 0 | 1.125 | 3.819 | 8.519 | 0 |
| BRAWH2000686 | 0 | 0 | 0 | 10.411 | 4.417 | 4.927 | 0 |
| BRAWH2000697 | 0 | 0 | 0 | 4.308 | 7.310 | 1.019 | 0 |
| BRAWH2000839 | 0 | 2.883 | 0 | 1.749 | 1.484 | 8.277 | 0 |
| BRAWH2000924 | 0 | 0 | 0 | 1.222 | 8.296 | 4.048 | 0 |
| BRAWH2001019 | 0 | 0 | 0 | 0 | 0 | 11.490 | 0 |
| BRAWH2001037 | 0 | 0 | 0 | 0 | 0 | 4.041 | 0 |
| BRAWH2001092 | 0 | 0 | 2.711 | 1.247 | 0 | 0.531 | 0 |
| BRAWH2001141 | 0 | 0 | 0 | 10.821 | 0 | 5.121 | 0 |
| BRAWH2001166 | 0 | 0 | 24.344 | 0 | 0 | 0 | 0 |
| BRAWH2001171 | 0 | 0 | 4.763 | 9.644 | 0 | 9.334 | 0 |
| BRAWH2001186 | 0 | 0 | 0 | 0 | 0 | 2.871 | 0 |
| BRAWH2001238 | 0 | 0 | 0 | 42.908 | 0 | 0 | 0 |
| BRAWH2001321 | 0 | 0 | 0 | 3.656 | 0 | 15.569 | 0 |
| BRAWH2001395 | 0 | 0 | 0 | 3.580 | 3.037 | 2.541 | 0 |
| BRAWH2001418 | 0 | 0 | 0 | 3.661 | 9.319 | 2.599 | 0 |
| BRAWH2001439 | 0 | 0 | 0 | 2.921 | 0 | 0 | 0 |
| BRAWH2001459 | 0 | 0 | 0 | 5.208 | 0 | 0 | 0 |
| BRAWH2001461 | 0 | 0 | 0 | 0 | 0 | 3.582 | 0 |
| BRAWH2001535 | 0 | 0 | 0 | 0 | 5.352 | 6.965 | 0 |
| BRAWH2001662 | 0 | 0 | 0 | 3.263 | 0 | 0 | 0 |
| BRAWH2001671 | 0 | 0 | 0 | 8.097 | 0 | 0 | 0 |
| BRAWH2002047 | 0 | 0 | 0 | 41.512 | 0 | 0 | 0 |
| BRAWH2002060 | 0 | 0 | 0 | 41.512 | 0 | 0 | 0 |
| BRAWH2002318 | 0 | 0 | 0 | 0 | 0 | 26.889 | 0 |
| BRAWH2002549 | 0 | 0 | 0 | 0 | 0 | 18.154 | 0 |
| BRAWH2002560 | 0 | 0 | 0 | 0 | 0 | 6.503 | 0 |
| BRAWH2002601 | 0 | 0 | 0 | 3.530 | 0 | 0 | 0 |
| BRAWH2002811 | 0 | 0 | 0 | 0 | 31.162 | 17.379 | 0 |
| BRAWH2002911 | 0 | 0 | 0 | 16.889 | 0 | 0 | 0 |
| BRAWH2002963 | 0 | 0 | 0 | 8.469 | 0 | 0 | 0 |
| BRAWH2003000 | 0 | 0 | 0 | 41.512 | 0 | 0 | 0 |
| BRAWH2003025 | 0 | 0 | 0 | 0 | 10.956 | 3.055 | 0 |
| BRAWH2003355 | 0 | 0 | 0 | 0 | 78.324 | 0 | 0 |
| BRAWH2003662 | 0 | 0 | 0 | 8.798 | 0 | 18.734 | 0 |
| BRAWH2004078 | 0 | 0 | 0 | 3.740 | 0 | 0 | 0 |
| BRAWH2004095 | 0 | 0 | 0 | 9.229 | 4.271 | 5.956 | 0 |
| BRAWH2004779 | 0 | 0 | 0 | 0 | 0 | 50.189 | 0 |
| BRAWH2004842 | 0 | 0 | 0 | 11.316 | 0 | 8.032 | 0 |
| BRAWH2005008 | 0 | 0 | 0 | 11.719 | 0 | 0 | 0 |
| BRAWH2005068 | 12.608 | 0 | 0 | 0.571 | 1.453 | 0.405 | 0 |
| BRAWH2005661 | 0 | 11.221 | 0 | 2.269 | 0 | 0 | 0 |
| BRAWH2006044 | 0 | 0 | 0 | 12.487 | 0 | 0 | 0 |
| BRAWH2006207 | 0 | 0 | 0 | 9.187 | 0 | 0 | 0 |
| BRAWH2006301 | 0 | 0 | 0 | 41.512 | 0 | 0 | 0 |
| BRAWH2006493 | 0 | 0 | 0 | 3.610 | 0 | 10.250 | 0 |
| BRAWH2006543 | 0 | 0 | 0 | 9.340 | 0 | 4.420 | 0 |
| BRAWH2006622 | 0 | 0 | 0 | 23.488 | 0 | 0 | 0 |
| BRAWH2006989 | 0 | 0 | 0 | 17.726 | 0 | 0 | 0 |
| BRAWH2006996 | 0 | 0 | 0 | 7.243 | 0 | 0 | 0 |
| BRAWH2007591 | 0 | 0 | 0 | 22.274 | 0 | 6.324 | 0 |
| BRAWH2007605 | 0 | 0 | 15.818 | 11.647 | 0 | 0 | 0 |
| BRAWH2007664 | 0 | 0 | 0 | 0 | 0 | 10.310 | 0 |
| BRAWH2007825 | 0 | 0 | 0 | 41.512 | 0 | 0 | 0 |
| BRAWH2008255 | 0 | 0 | 0 | 0 | 0 | 4.257 | 0 |
| BRAWH2008706 | 0 | 0 | 0 | 0 | 0 | 5.795 | 0 |
| BRAWH2008903 | 0 | 0 | 0 | 0 | 0 | 4.260 | 0 |
| BRAWH2008956 | 0 | 0 | 0 | 22.103 | 0 | 0 | 0 |
| BRAWH2008993 | 0 | 0 | 0 | 0 | 0 | 50.189 | 0 |
| BRAWH2009238 | 0 | 0 | 0 | 7.400 | 0 | 5.253 | 0 |
| BRAWH2009307 | 0 | 0 | 0 | 4.240 | 0 | 0 | 0 |
| BRAWH2009695 | 0 | 0 | 0 | 18.990 | 0 | 26.958 | 0 |
| BRAWH2010136 | 0 | 0 | 0 | 0 | 0 | 26.605 | 0 |
| BRAWH2010329 | 0 | 0 | 0 | 21.311 | 0 | 10.085 | 0 |
| BRAWH2010354 | 0 | 0 | 0 | 9.211 | 0 | 0 | 0 |
| BRAWH2010364 | 0 | 0 | 0 | 36.692 | 0 | 0 | 0 |
| BRAWH2010552 | 0 | 0 | 0 | 41.512 | 0 | 0 | 0 |
| BRAWH2010754 | 0 | 0 | 0 | 7.940 | 0 | 0 | 0 |
| BRAWH2011204 | 0 | 0 | 0 | 0 | 0 | 50.189 | 0 |
| BRAWH2011343 | 0 | 0 | 0 | 9.958 | 0 | 0 | 0 |
| BRAWH2011812 | 0 | 0 | 0 | 0 | 0 | 13.646 | 0 |
| BRAWH2011920 | 0 | 0 | 0 | 0 | 0 | 38.183 | 0 |
| BRAWH2012164 | 0 | 0 | 0 | 14.788 | 0 | 0 | 0 |
| BRAWH2012538 | 0 | 35.605 | 0 | 0 | 0 | 0 | 0 |
| BRAWH2012698 | 0 | 0 | 0 | 0 | 0 | 57.958 | 0 |
| BRAWH2012827 | 0 | 0 | 0 | 0 | 0 | 9.061 | 0 |
| BRAWH2012866 | 0 | 0 | 0 | 3.017 | 0 | 14.993 | 0 |
| BRAWH2013047 | 0 | 0 | 0 | 13.334 | 67.880 | 0 | 0 |
| BRAWH2013219 | 0 | 0 | 0 | 0 | 35.004 | 9.761 | 0 |
| BRAWH2013294 | 0 | 0 | 0 | 13.997 | 0 | 0 | 0 |
| BRAWH2013748 | 0 | 0 | 0 | 0 | 0 | 8.068 | 0 |
| BRAWH2014053 | 0 | 0 | 0 | 16.482 | 0 | 0 | 0 |
| BRAWH2014188 | 0 | 0 | 0 | 0 | 0 | 2.164 | 0 |
| BRAWH2014414 | 0 | 0 | 0 | 0 | 0 | 54.002 | 0 |
| BRAWH2014511 | 0 | 0 | 0 | 3.050 | 0 | 12.988 | 0 |
| BRAWH2014645 | 0 | 7.420 | 0 | 3.751 | 0 | 2.130 | 0 |
| BRAWH2014662 | 0 | 0 | 0 | 11.016 | 0 | 0 | 0 |
| BRAWH2014876 | 0 | 0 | 0 | 7.433 | 0 | 0 | 0 |
| BRAWH2014954 | 0 | 0 | 0 | 41.512 | 0 | 0 | 0 |
| BRAWH2015247 | 0 | 0 | 0 | 7.410 | 37.723 | 0 | 0 |
| BRAWH2015385 | 0 | 0 | 0 | 41.512 | 0 | 0 | 0 |
| BRAWH2015728 | 0 | 0 | 0 | 0 | 0 | 50.189 | 0 |
| BRAWH2016028 | 0 | 0 | 0 | 0 | 0 | 50.189 | 0 |
| BRAWH2016166 | 0 | 0 | 0 | 5.368 | 0 | 7.620 | 0 |
| BRAWH2016209 | 0 | 0 | 0 | 11.791 | 0 | 0 | 0 |
| BRAWH2016269 | 0 | 0 | 0 | 0.936 | 0 | 1.328 | 0 |
| BRAWH2016679 | 0 | 0 | 0 | 6.839 | 0 | 0 | 0 |
| BRAWH2016702 | 0 | 0 | 0 | 26.193 | 0 | 0 | 0 |
| BRAWH2017002 | 0 | 0 | 0 | 1.766 | 0 | 15.044 | 0 |
| BRAWH2017304 | 0 | 0 | 0 | 23.571 | 0 | 0 | 0 |
| BRAWH2017379 | 0 | 0 | 0 | 0 | 0 | 15.310 | 0 |
| BRAWH2017685 | 0 | 0 | 3.914 | 1.081 | 0 | 1.534 | 0 |
| BRAWH2018267 | 0 | 0 | 0 | 14.668 | 0 | 0 | 0 |
| BRAWH2018526 | 0 | 2.160 | 2.373 | 0.218 | 1.112 | 1.550 | 0 |
| BRAWH2018875 | 0 | 0 | 0 | 18.805 | 0 | 0 | 0 |
| BRAWH3000088 | 0 | 0 | 0 | 0 | 0 | 15.152 | 0 |
| BRAWH3001319 | 0 | 0 | 0 | 19.373 | 0 | 0 | 0 |
| BRAWH3001891 | 0 | 0 | 0 | 36.772 | 0 | 17.401 | 0 |
| BRAWH3001918 | 0 | 0 | 0 | 39.423 | 0 | 0 | 0 |
| BRAWH3002600 | 0 | 0 | 0 | 26.120 | 0 | 37.080 | 0 |
| BRAWH3002853 | 0 | 0 | 5.704 | 2.100 | 0 | 2.236 | 0 |
| BRAWH3003573 | 0 | 0 | 0 | 14.310 | 0 | 0 | 0 |
| BRAWH3003727 | 0 | 0 | 0 | 21.411 | 0 | 0 | 0 |
| BRAWH3004530 | 0 | 0 | 0 | 25.041 | 0 | 0 | 0 |
| BRAWH3005132 | 0 | 0 | 0 | 0 | 0 | 50.189 | 0 |
| BRAWH3005146 | 0 | 0 | 0 | 3.594 | 0 | 0 | 0 |
| BRAWH3006761 | 0 | 0 | 0 | 0 | 0 | 21.430 | 0 |
| BRAWH3007129 | 0 | 0 | 0 | 13.969 | 35.557 | 0 | 0 |
| BRAWH3008931 | 0 | 0 | 0 | 4.916 | 0 | 6.980 | 0 |
| BRAWH3009285 | 0 | 0 | 0 | 26.193 | 0 | 0 | 0 |
| BRAWH3009297 | 0 | 0 | 0 | 41.512 | 0 | 0 | 0 |
| BRAWH3010461 | 0 | 0 | 0 | 10.207 | 0 | 14.490 | 0 |
| BRAWH3016271 | 0 | 0 | 0 | 0 | 0 | 5.435 | 0 |
| BRAWH3017259 | 0 | 0 | 0 | 4.967 | 0 | 0 | 0 |
| BRAWH3017980 | 0 | 0 | 0 | 0 | 0 | 50.189 | 0 |
| BRAWH3018063 | 0 | 0 | 0 | 0 | 0 | 7.978 | 0 |
| BRAWH3021545 | 0 | 0 | 0 | 2.589 | 0 | 0 | 0 |
| BRAWH3021574 | 0 | 0 | 0 | 3.336 | 0 | 0 | 0 |
| BRAWH3027440 | 0 | 0 | 0 | 41.512 | 0 | 0 | 0 |
| BRAWH3029556 | 0 | 0 | 0 | 0 | 0 | 19.615 | 0 |
| BRAWH3029806 | 0 | 0 | 0 | 0 | 0 | 8.979 | 0 |
| BRAWH3030810 | 0 | 0 | 0 | 67.989 | 0 | 10.724 | 0 |
| BRAWH3034743 | 0 | 0 | 0 | 5.913 | 0 | 12.592 | 0 |
| BRAWH3035403 | 0 | 0 | 0 | 25.081 | 0 | 0 | 0 |
| BRCAN1000076 | 0 | 0 | 90.793 | 0 | 0 | 0 | 0 |
| BRCAN1000168 | 0 | 0 | 0 | 1.001 | 0 | 11.371 | 0 |
| BRCAN2001223 | 0 | 0 | 0 | 0 | 0 | 23.313 | 0 |
| BRCAN2002173 | 0 | 0 | 0 | 2.823 | 7.185 | 2.004 | 0 |
| BRCAN2003944 | 0 | 1.977 | 0 | 0 | 5.088 | 0.851 | 0 |
| BRCAN2006019 | 0 | 0 | 0 | 2.666 | 13.575 | 0 | 0 |
| BRCAN2006117 | 0 | 0 | 0 | 17.693 | 0 | 25.117 | 0 |
| BRCAN2006401 | 0 | 0 | 0 | 0 | 0 | 22.844 | 0 |
| BRCAN2007700 | 0 | 0 | 0 | 8.632 | 0 | 0 | 0 |
| BRCAN2008894 | 0 | 0 | 0 | 0 | 82.533 | 0 | 0 |
| BRCAN2009432 | 0 | 0 | 0 | 1.869 | 0 | 0 | 0 |
| BRCAN2010374 | 0 | 0 | 0 | 0 | 0 | 30.516 | 0 |
| BRCAN2010376 | 0 | 0 | 0 | 23.627 | 0 | 0 | 0 |
| BRCAN2010665 | 0 | 0 | 0 | 0 | 0 | 9.879 | 0 |
| BRCAN2014881 | 0 | 0 | 0 | 0 | 0 | 30.516 | 0 |
| BRCAN2015371 | 0 | 0 | 0 | 15.560 | 0 | 0 | 0 |
| BRCAN2015719 | 0 | 0 | 0 | 12.858 | 0 | 0 | 0 |
| BRCAN2016046 | 0 | 0 | 0 | 0 | 0 | 30.516 | 0 |
| BRCAN2019653 | 0 | 0 | 0 | 0 | 22.535 | 0 | 0 |
| BRCAN2020710 | 0 | 0 | 0 | 7.714 | 0 | 0 | 0 |
| BRCAN2022126 | 0 | 0 | 0 | 3.446 | 0 | 14.677 | 0 |
| BRCAN2024572 | 0 | 0 | 0 | 0 | 0 | 5.504 | 0 |
| BRCAN2025368 | 0 | 0 | 0 | 0 | 61.165 | 0 | 0 |
| BRCAN2026197 | 0 | 0 | 0 | 5.541 | 0 | 4.719 | 0 |
| BRCAN2026340 | 0 | 0 | 0 | 10.726 | 54.604 | 0 | 0 |
| BRCAN2027334 | 0 | 0 | 0 | 0 | 0 | 30.516 | 0 |
| BRCAN2027364 | 0 | 0 | 0 | 0 | 10.731 | 5.985 | 0 |
| BRCAN2028036 | 0 | 0 | 0 | 0 | 8.724 | 4.865 | 0 |
| BRCAN2028355 | 46.308 | 0 | 0 | 0 | 0 | 0 | 0 |
| BRCOC1000040 | 0 | 0 | 0 | 0 | 0 | 5.943 | 0 |
| BRCOC1000094 | 0 | 0 | 0 | 0.676 | 17.202 | 3.838 | 0 |
| BRCOC2000004 | 0 | 0 | 0 | 0 | 0 | 9.069 | 0 |
| BRCOC2000047 | 0 | 0 | 0 | 0 | 50.495 | 0 | 0 |
| BRCOC2000186 | 0 | 0 | 0 | 4.222 | 0 | 0 | 0 |
| BRCOC2000333 | 0 | 0 | 0 | 0 | 43.281 | 0 | 0 |
| BRCOC2000670 | 0 | 0 | 0 | 0 | 0 | 22.144 | 0 |
| BRCOC2003100 | 0 | 0 | 0 | 16.691 | 0 | 0 | 0 |
| BRCOC2003187 | 0 | 0 | 0 | 0.558 | 0 | 0 | 0 |
| BRCOC2003213 | 0 | 0 | 0 | 7.368 | 0 | 0 | 0 |
| BRCOC2004036 | 0 | 0 | 0 | 0 | 50.495 | 0 | 0 |
| BRCOC2005542 | 0 | 0 | 0 | 0 | 0 | 17.805 | 0 |
| BRCOC2005903 | 0 | 0 | 0 | 0 | 0 | 8.845 | 0 |
| BRCOC2006189 | 0 | 0 | 0 | 0 | 11.994 | 6.689 | 0 |
| BRCOC2006270 | 0 | 0 | 0 | 0 | 50.495 | 0 | 0 |
| BRCOC2007034 | 0 | 0 | 0 | 16.691 | 0 | 0 | 0 |
| BRCOC2009937 | 0 | 0 | 0 | 0 | 0 | 16.189 | 0 |
| BRCOC2011506 | 0 | 0 | 0 | 0 | 0 | 0.959 | 0 |
| BRCOC2012172 | 0 | 0 | 0 | 1.996 | 0 | 0 | 0 |
| BRCOC2012551 | 0 | 0 | 0 | 5.119 | 0 | 7.267 | 0 |
| BRCOC2013780 | 0 | 0 | 0 | 0 | 50.495 | 0 | 0 |
| BRCOC2019841 | 0 | 0 | 0 | 16.691 | 0 | 0 | 0 |
| BRCOC2019896 | 0 | 0 | 0 | 0 | 0 | 22.144 | 0 |
| BRCOC2019910 | 0 | 0 | 0 | 15.968 | 2.258 | 8.816 | 0 |
| BRHIP1000104 | 0 | 0 | 5.041 | 2.320 | 9.447 | 5.928 | 0 |
| BRHIP1000108 | 0 | 0 | 0 | 0 | 15.572 | 4.342 | 0 |
| BRHIP1000129 | 0 | 0 | 0 | 0 | 0 | 49.688 | 0 |
| BRHIP1000174 | 0 | 0 | 0 | 3.598 | 0 | 5.108 | 0 |
| BRHIP2000087 | 0 | 0 | 0 | 0 | 23.781 | 6.632 | 0 |
| BRHIP2000210 | 0 | 0 | 0 | 41.027 | 0 | 0 | 0 |
| BRHIP2000312 | 0 | 0 | 0 | 9.205 | 0 | 6.534 | 0 |
| BRHIP2000534 | 0 | 2.402 | 0 | 0 | 0 | 0 | 0 |
| BRHIP2000553 | 0 | 0 | 0 | 1.824 | 0 | 2.589 | 0 |
| BRHIP2000920 | 0 | 0 | 0 | 25.999 | 0 | 0 | 0 |
| BRHIP2001099 | 0 | 0 | 0 | 4.920 | 0 | 0 | 0 |
| BRHIP2001886 | 0 | 0 | 0 | 0 | 0 | 49.688 | 0 |
| BRHIP2002143 | 0 | 0 | 0 | 41.027 | 0 | 0 | 0 |
| BRHIP2002339 | 0 | 0 | 0 | 0 | 0 | 15.815 | 0 |
| BRHIP2003062 | 0 | 0 | 0 | 6.742 | 0 | 0 | 0 |
| BRHIP2004359 | 0 | 0 | 0 | 34.560 | 0 | 0 | 0 |
| BRHIP2005010 | 0 | 0 | 0 | 11.189 | 56.963 | 0 | 0 |
| BRHIP2005271 | 0 | 0 | 0 | 0 | 0 | 7.137 | 0 |
| BRHIP2005752 | 0 | 0 | 0 | 41.027 | 0 | 0 | 0 |
| BRHIP2007305 | 0 | 7.946 | 0 | 0 | 0 | 3.422 | 0 |
| BRHIP2007741 | 0 | 0 | 0 | 3.696 | 0 | 8.745 | 0 |
| BRHIP2008607 | 0 | 0 | 0 | 25.490 | 0 | 0 | 0 |
| BRHIP2009019 | 0 | 0 | 0 | 41.027 | 0 | 0 | 0 |
| BRHIP2010217 | 0 | 0 | 0 | 0 | 0 | 5.627 | 0 |
| BRHIP2010487 | 0 | 0 | 0 | 0 | 0 | 4.144 | 0 |
| BRHIP2010571 | 0 | 0 | 0 | 21.859 | 0 | 15.515 | 0 |
| BRHIP2010723 | 0 | 0 | 0 | 0 | 0 | 49.688 | 0 |
| BRHIP2011120 | 0 | 0 | 0 | 32.686 | 0 | 7.734 | 0 |
| BRHIP2011491 | 0 | 0 | 0 | 0 | 8.960 | 0 | 0 |
| BRHIP2011576 | 0 | 0 | 0 | 13.607 | 0 | 0 | 0 |
| BRHIP2013699 | 0 | 0 | 0 | 15.145 | 0 | 0 | 0 |
| BRHIP2014228 | 0 | 0 | 0 | 10.447 | 0 | 0 | 0 |
| BRHIP2015153 | 0 | 0 | 0 | 14.222 | 0 | 10.095 | 0 |
| BRHIP2015224 | 0 | 0 | 0 | 0 | 0 | 9.167 | 0 |
| BRHIP2015245 | 0 | 0 | 0 | 5.055 | 0 | 2.392 | 0 |
| BRHIP2015679 | 0 | 0 | 0 | 3.561 | 0 | 0 | 0 |
| BRHIP2017642 | 0 | 0 | 0 | 0 | 0 | 13.013 | 0 |
| BRHIP2017945 | 0 | 22.622 | 0 | 0 | 0 | 0 | 0 |
| BRHIP2018712 | 0 | 0 | 0 | 0 | 0 | 6.122 | 0 |
| BRHIP2018719 | 0 | 0 | 0 | 0 | 0 | 49.688 | 0 |
| BRHIP2019186 | 0 | 0 | 0 | 0 | 0 | 3.977 | 0 |
| BRHIP2019494 | 0 | 0 | 0 | 25.807 | 0 | 0 | 0 |
| BRHIP2019819 | 0 | 0 | 0 | 30.770 | 0 | 0 | 0 |
| BRHIP2020573 | 0 | 0 | 0 | 6.423 | 0 | 0 | 0 |
| BRHIP2020622 | 0 | 0 | 0 | 0 | 0 | 23.313 | 0 |
| BRHIP2020799 | 0 | 0 | 0 | 3.592 | 0 | 15.298 | 0 |
| BRHIP2021495 | 0 | 0 | 0 | 4.552 | 46.351 | 0 | 0 |
| BRHIP2021762 | 0 | 19.554 | 0 | 7.910 | 0 | 0 | 0 |
| BRHIP2021858 | 0 | 0 | 0 | 4.750 | 0 | 3.372 | 0 |
| BRHIP2021870 | 0 | 0 | 0 | 9.454 | 0 | 0 | 0 |
| BRHIP2022054 | 0 | 0 | 0 | 1.748 | 0 | 0 | 0 |
| BRHIP2022326 | 0 | 0 | 0 | 1.811 | 0 | 1.714 | 0 |
| BRHIP2022986 | 0 | 0 | 0 | 4.039 | 0 | 0 | 0 |
| BRHIP2023071 | 0 | 0 | 0 | 0 | 0 | 49.688 | 0 |
| BRHIP2023309 | 0 | 0 | 0 | 8.280 | 0 | 0 | 0 |
| BRHIP2023762 | 0 | 0 | 0 | 0 | 8.623 | 7.214 | 0 |
| BRHIP2023888 | 0 | 2.583 | 2.838 | 2.089 | 0 | 1.854 | 0 |
| BRHIP2024398 | 0 | 0 | 0 | 11.952 | 0 | 0 | 0 |
| BRHIP2024742 | 0 | 8.754 | 0 | 1.770 | 0 | 1.257 | 0 |
| BRHIP2025448 | 0 | 0 | 0 | 0 | 0 | 30.812 | 0 |
| BRHIP2025679 | 0 | 0 | 0 | 41.027 | 0 | 0 | 0 |
| BRHIP2026061 | 0 | 0 | 0 | 0 | 0 | 30.812 | 0 |
| BRHIP2026288 | 0 | 0 | 0 | 0 | 77.982 | 0 | 0 |
| BRHIP2027077 | 0 | 0 | 0 | 0 | 0 | 2.035 | 0 |
| BRHIP2027685 | 0 | 0 | 0 | 0 | 0 | 49.688 | 0 |
| BRHIP2028187 | 0 | 0 | 0 | 25.807 | 0 | 0 | 0 |
| BRHIP2028583 | 0 | 87.309 | 0 | 0 | 0 | 0 | 0 |
| BRHIP2029643 | 0 | 0 | 0 | 10.603 | 0 | 0 | 0 |
| BRHIP3000111 | 0 | 0 | 0 | 0.727 | 3.699 | 2.063 | 0 |
| BRHIP3000131 | 0 | 0 | 0 | 5.213 | 0 | 0 | 0 |
| BRHIP3000240 | 0 | 0 | 0 | 67.607 | 0 | 0 | 0 |
| BRHIP3000377 | 0 | 80.864 | 0 | 0 | 0 | 0 | 0 |
| BRHIP3000457 | 0 | 0 | 0 | 38.553 | 0 | 0 | 0 |
| BRHIP3001283 | 0 | 0 | 0 | 0 | 0 | 49.688 | 0 |
| BRHIP3001360 | 0 | 0 | 0 | 41.027 | 0 | 0 | 0 |
| BRHIP3002114 | 0 | 0 | 0 | 27.684 | 0 | 0 | 0 |
| BRHIP3002691 | 0 | 0 | 0 | 0 | 0 | 8.357 | 0 |
| BRHIP3003063 | 0 | 0 | 0 | 0 | 0 | 33.130 | 0 |
| BRHIP3003126 | 0 | 0 | 0 | 25.807 | 0 | 0 | 0 |
| BRHIP3003961 | 0 | 0 | 0 | 13.462 | 0 | 0 | 0 |
| BRHIP3003984 | 0 | 0 | 0 | 29.254 | 0 | 0 | 0 |
| BRHIP3004215 | 0 | 0 | 0 | 9.636 | 0 | 13.679 | 0 |
| BRHIP3004968 | 0 | 0 | 0 | 23.879 | 0 | 0 | 0 |
| BRHIP3005574 | 0 | 0 | 0 | 10 | 0 | 0 | 0 |
| BRHIP3005673 | 0 | 0 | 0 | 0 | 0 | 25.956 | 0 |
| BRHIP3005801 | 0 | 0 | 0 | 13.302 | 0 | 0 | 0 |
| BRHIP3006717 | 0 | 0 | 0 | 0 | 6.150 | 5.145 | 0 |
| BRHIP3007223 | 0 | 0 | 0 | 1.431 | 0 | 0 | 0 |
| BRHIP3007586 | 0 | 0 | 0 | 7.348 | 0 | 3.477 | 0 |
| BRHIP3007640 | 0 | 0 | 0 | 31.684 | 0 | 0 | 0 |
| BRHIP3008405 | 0 | 0 | 0 | 12.487 | 0 | 0 | 0 |
| BRHIP3012736 | 0 | 0 | 0 | 0 | 0 | 66.389 | 0 |
| BRHIP3014675 | 0 | 0 | 0 | 26.918 | 0 | 0 | 0 |
| BRHIP3017146 | 0 | 0 | 0 | 0 | 0 | 33.057 | 0 |
| BRHIP3017855 | 0 | 0 | 0 | 13.588 | 0 | 0 | 0 |
| BRHIP3018278 | 0 | 0 | 0 | 27.684 | 0 | 0 | 0 |
| BRHIP3020046 | 0 | 0 | 0 | 0 | 0 | 1.021 | 0 |
| BRHIP3021019 | 0 | 0 | 0 | 0 | 0 | 7.803 | 0 |
| BRHIP3026052 | 0 | 0 | 0 | 0 | 54.140 | 0 | 0 |
| BRHIP3027594 | 0 | 0 | 0 | 0 | 0 | 39.173 | 0 |
| BRSSN2000097 | 0 | 48.599 | 0 | 0 | 0 | 0 | 0 |
| BRSSN2000293 | 0 | 0 | 0 | 2.328 | 0 | 3.304 | 0 |
| BRSSN2000295 | 0 | 0 | 0 | 9.831 | 0 | 0 | 0 |
| BRSSN2000312 | 0 | 0 | 0 | 7.636 | 0 | 21.682 | 0 |
| BRSSN2000498 | 0 | 7.680 | 0 | 1.553 | 3.954 | 4.410 | 0 |
| BRSSN2000561 | 0 | 0 | 0 | 0 | 0 | 12.636 | 0 |
| BRSSN2000566 | 0 | 0 | 0 | 6.905 | 17.576 | 9.802 | 0 |
| BRSSN2000832 | 0 | 0 | 0 | 8.545 | 0 | 0 | 0 |
| BRSSN2001758 | 0 | 0 | 0 | 0 | 33.969 | 9.472 | 0 |
| BRSSN2001869 | 0 | 0 | 0 | 2.142 | 0 | 0 | 0 |
| BRSSN2003012 | 0 | 0 | 0 | 0 | 15.631 | 0 | 0 |
| BRSSN2004496 | 0 | 0 | 0 | 0 | 0 | 21.304 | 0 |
| BRSSN2006611 | 0 | 0 | 0 | 0 | 0 | 21.304 | 0 |
| BRSSN2006892 | 0 | 0 | 0 | 10.990 | 0 | 0 | 0 |
| BRSSN2007338 | 0 | 0 | 0 | 0 | 26.707 | 0 | 0 |
| BRSSN2009518 | 0 | 0 | 0 | 0 | 0 | 1.736 | 0 |
| BRSSN2010587 | 0 | 0 | 0 | 16.015 | 0 | 0 | 0 |
| BRSSN2010596 | 0 | 0 | 0 | 0 | 43.310 | 12.077 | 0 |
| BRSSN2011262 | 0 | 0 | 0 | 2.000 | 0 | 0 | 0 |
| BRSSN2011738 | 0 | 0 | 0 | 7.465 | 0 | 0 | 0 |
| BRSSN2011843 | 0 | 0 | 0 | 8.495 | 0 | 0 | 0 |
| BRSSN2013696 | 0 | 0 | 0 | 12.675 | 0 | 0 | 0 |
| BRSSN2013733 | 0 | 0 | 0 | 0 | 0 | 21.304 | 0 |
| BRSSN2014294 | 0 | 65.348 | 0 | 0 | 0 | 0 | 0 |
| BRSSN2014299 | 0 | 0 | 0 | 2.623 | 0 | 0 | 0 |
| BRSSN2014610 | 0 | 0 | 0 | 5.068 | 0 | 3.597 | 0 |
| BRSSN2014685 | 0 | 0 | 0 | 0 | 0 | 14.308 | 0 |
| BRSSN2015199 | 0 | 0 | 0 | 16.015 | 0 | 0 | 0 |
| BRSSN2015238 | 0 | 0 | 0 | 4.859 | 0 | 0 | 0 |
| BRSSN2015493 | 0 | 0 | 0 | 7.783 | 0 | 0 | 0 |
| BRSSN2018447 | 0 | 0 | 0 | 4.638 | 0 | 6.585 | 0 |
| BRSTN1000083 | 0 | 0 | 0 | 1.360 | 0 | 1.930 | 0 |
| BRSTN2000058 | 0 | 0 | 0 | 3.424 | 0 | 2.431 | 0 |
| BRSTN2000220 | 0 | 0 | 0 | 0 | 0 | 20.742 | 0 |
| BRSTN2006580 | 0 | 0 | 0 | 16.447 | 0 | 0 | 0 |
| BRSTN2008052 | 0 | 0 | 0 | 15.541 | 0 | 0 | 0 |
| BRSTN2010089 | 0 | 0 | 0 | 4.683 | 0 | 0 | 0 |
| BRSTN2010107 | 0 | 0 | 0 | 0.950 | 6.044 | 6.067 | 0 |
| BRSTN2010750 | 0 | 0 | 0 | 7.220 | 0 | 30.747 | 0 |
| BRSTN2011597 | 0 | 0 | 0 | 16.447 | 0 | 0 | 0 |
| BRSTN2011961 | 0 | 0 | 2.233 | 3.083 | 0 | 0 | 0 |
| BRSTN2012069 | 1.482 | 2.124 | 0.073 | 0.517 | 0.718 | 0.029 | 7.145 |
| BRSTN2012089 | 0 | 0 | 0 | 4.117 | 20.959 | 5.845 | 0 |
| BRSTN2012174 | 0 | 2.839 | 0 | 1.292 | 0.731 | 0.611 | 0 |
| BRSTN2012451 | 0 | 0 | 0 | 0 | 0 | 0.832 | 0 |
| BRSTN2013502 | 0 | 0 | 0 | 6.900 | 0 | 0 | 0 |
| BRSTN2013931 | 0 | 0 | 0 | 0.686 | 0 | 1.947 | 0 |
| BRSTN2015015 | 0 | 0 | 0 | 19.897 | 0 | 0 | 0 |
| BRSTN2015889 | 0 | 27.188 | 0 | 5.499 | 0 | 7.806 | 0 |
| BRSTN2015978 | 0 | 0 | 0 | 0.991 | 5.372 | 3.057 | 22.920 |
| BRSTN2016470 | 0 | 0 | 3.376 | 7.146 | 7.909 | 0.441 | 0 |
| BRSTN2016992 | 0 | 3.454 | 0 | 5.588 | 40.898 | 4.958 | 0 |
| BRTHA1000021 | 0 | 0 | 0 | 24.379 | 0 | 0 | 0 |
| BRTHA1000311 | 0 | 0 | 0 | 10.472 | 0 | 14.866 | 0 |
| BRTHA2001812 | 0 | 59.819 | 0 | 0 | 0 | 0 | 0 |
| BRTHA2005013 | 0 | 78.321 | 0 | 0 | 0 | 0 | 0 |
| BRTHA2005864 | 0 | 0 | 0 | 3.455 | 0 | 0 | 0 |
| BRTHA2007998 | 0 | 0 | 0 | 0 | 0 | 1.738 | 0 |
| BRTHA2008955 | 0 | 0 | 0 | 39.025 | 0 | 0 | 0 |
| BRTHA2009486 | 0 | 76.725 | 0 | 0 | 0 | 0 | 0 |
| BRTHA2011194 | 0 | 0 | 0 | 39.025 | 0 | 0 | 0 |
| BRTHA2011641 | 0 | 0 | 0 | 0 | 0 | 47.605 | 0 |
| BRTHA2013054 | 0 | 0 | 0 | 4.264 | 0 | 6.053 | 0 |
| BRTHA2013540 | 0 | 0 | 0 | 0 | 0 | 34.151 | 0 |
| BRTHA2016577 | 0 | 0 | 0 | 0 | 0 | 4.755 | 0 |
| BRTHA2026071 | 0 | 0 | 0 | 0 | 0 | 12.368 | 0 |
| BRTHA2033155 | 0 | 0 | 0 | 9.056 | 0 | 4.285 | 0 |
| BRTHA2035743 | 0 | 0 | 0 | 4.816 | 0 | 0 | 0 |
| BRTHA3000789 | 0 | 0 | 0 | 25.001 | 0 | 0 | 0 |
| BRTHA3002427 | 0 | 0 | 0 | 2.029 | 0 | 5.761 | 0 |
| BRTHA3003023 | 0 | 0 | 0 | 0 | 0 | 4.073 | 0 |
| BRTHA3003490 | 0 | 0 | 0 | 2.304 | 0 | 0 | 0 |
| BRTHA3003704 | 0 | 0 | 0 | 39.025 | 0 | 0 | 0 |
| BRTHA3003736 | 0 | 0 | 0 | 1.527 | 0 | 0 | 0 |
| BRTHA3004307 | 0 | 0 | 0 | 10.691 | 0 | 6.071 | 0 |
| BRTHA3004432 | 0 | 0 | 0 | 0 | 0 | 32.330 | 0 |
| BRTHA3004475 | 0 | 0 | 0 | 0 | 0 | 47.605 | 0 |
| BRTHA3007685 | 0 | 0 | 0 | 39.025 | 0 | 0 | 0 |
| BRTHA3009090 | 0 | 0 | 0 | 8.354 | 0 | 11.859 | 0 |
| BRTHA3009858 | 0 | 0 | 0 | 16.462 | 0 | 0 | 0 |
| BRTHA3010530 | 0 | 0 | 0 | 19.846 | 0 | 0 | 0 |
| BRTHA3021708 | 0 | 0 | 0 | 39.025 | 0 | 0 | 0 |
| BRTHA3021971 | 0 | 0 | 0 | 0 | 0 | 1.956 | 0 |
| BRTHA3023403 | 0 | 0 | 0 | 0 | 0 | 7.485 | 0 |
| BRTHA3026507 | 0 | 0 | 0 | 4.960 | 0 | 0 | 0 |
| BRTHA3026916 | 0 | 0 | 0 | 0 | 0 | 47.605 | 0 |
| CD34C3000252 | 0 | 5.761 | 0 | 0.583 | 0 | 1.654 | 0 |
| CHONS2000172 | 0 | 0 | 0 | 0 | 13.499 | 0 | 0 |
| CHONS2002829 | 0 | 0 | 0 | 1.160 | 0 | 1.646 | 0 |
| COLON2000470 | 0 | 49.994 | 0 | 0 | 0 | 0 | 0 |
| COLON2001721 | 0 | 0 | 0 | 19.633 | 0 | 11.149 | 0 |
| CTONG1000010 | 0 | 0 | 0 | 0 | 0 | 6.532 | 0 |
| CTONG1000052 | 0 | 0 | 0 | 0.858 | 0 | 1.218 | 0 |
| CTONG1000087 | 0 | 0 | 0 | 0 | 0 | 3.161 | 0 |
| CTONG1000088 | 91.578 | 0 | 0 | 0 | 0 | 0 | 0 |
| CTONG1000094 | 0 | 0 | 0 | 1.106 | 1.407 | 2.747 | 0 |
| CTONG1000175 | 0 | 11.817 | 0 | 0 | 0 | 0 | 0 |
| CTONG1000218 | 0 | 0 | 0 | 1.025 | 1.740 | 0.970 | 0 |
| CTONG1000241 | 0 | 0 | 0 | 0 | 0 | 4.275 | 0 |
| CTONG2000063 | 0 | 0 | 0 | 1.519 | 0 | 4.314 | 0 |
| CTONG2000201 | 0 | 27.210 | 0 | 0 | 0 | 0 | 0 |
| CTONG2000508 | 0 | 11.086 | 0 | 1.682 | 0 | 3.183 | 0 |
| CTONG2001598 | 0 | 0 | 0 | 27.592 | 0 | 0 | 0 |
| CTONG2001748 | 0 | 0 | 0 | 1.484 | 0 | 6.319 | 0 |
| CTONG2002270 | 0 | 0 | 0 | 5.647 | 0 | 1.603 | 0 |
| CTONG2002520 | 0 | 0 | 0 | 0 | 0 | 8.898 | 0 |
| CTONG2002709 | 0 | 0 | 16.709 | 1.538 | 0 | 0 | 0 |
| CTONG2002744 | 0 | 0 | 0 | 0.836 | 0 | 1.187 | 0 |
| CTONG2002766 | 0 | 0 | 0 | 16.772 | 0 | 11.905 | 0 |
| CTONG2002803 | 0 | 0 | 0 | 2.090 | 0 | 2.967 | 0 |
| CTONG2002841 | 0 | 0 | 0 | 15.036 | 0 | 0 | 0 |
| CTONG2003094 | 0 | 0 | 0 | 1.300 | 0 | 3.692 | 0 |
| CTONG2003298 | 0 | 0 | 0 | 0.515 | 0 | 2.194 | 0 |
| CTONG2003524 | 0 | 0 | 0 | 3.431 | 0 | 3.247 | 0 |
| CTONG2003699 | 0 | 0 | 0 | 0 | 0 | 35.106 | 0 |
| CTONG2004115 | 0 | 0 | 0 | 8.483 | 0 | 0 | 0 |
| CTONG2004423 | 0 | 0 | 0 | 8.156 | 0 | 0 | 0 |
| CTONG2004487 | 0 | 0 | 0 | 6.714 | 0 | 4.765 | 0 |
| CTONG2005028 | 0 | 0 | 0 | 14.575 | 0 | 0 | 0 |
| CTONG2005553 | 0 | 0 | 0 | 0.508 | 0 | 0.722 | 0 |
| CTONG2005615 | 0 | 0 | 0 | 3.375 | 0 | 0 | 0 |
| CTONG2006004 | 0 | 7.408 | 0 | 0.749 | 0 | 0 | 0 |
| CTONG2006235 | 0 | 0 | 0 | 1.163 | 5.919 | 4.952 | 0 |
| CTONG2006562 | 0 | 0 | 0 | 4.969 | 0 | 1.411 | 0 |
| CTONG2006611 | 0 | 0 | 0 | 0 | 0 | 7.276 | 0 |
| CTONG2006709 | 0 | 0 | 0 | 1.835 | 0 | 0 | 0 |
| CTONG2006932 | 0 | 0 | 0 | 0.781 | 0 | 1.109 | 0 |
| CTONG2007007 | 0 | 0 | 0 | 0 | 3.364 | 0 | 0 |
| CTONG2007072 | 0 | 0 | 0 | 0 | 7.773 | 0 | 0 |
| CTONG2007078 | 0 | 0 | 0 | 5.442 | 0 | 1.288 | 0 |
| CTONG2007168 | 0 | 0 | 0 | 12.872 | 0 | 0 | 0 |
| CTONG2007400 | 0 | 0 | 0 | 0 | 0 | 11.594 | 0 |
| CTONG2007474 | 0 | 0 | 0 | 7.539 | 0 | 0 | 0 |
| CTONG2007500 | 0 | 5.278 | 0 | 0 | 0 | 5.304 | 0 |
| CTONG2007681 | 0 | 0 | 0 | 26.233 | 0 | 0 | 0 |
| CTONG2007776 | 0 | 0 | 0 | 6.038 | 0 | 0 | 0 |
| CTONG2008398 | 0 | 5.366 | 0 | 0.543 | 0 | 2.311 | 0 |
| CTONG2008405 | 0 | 0 | 0 | 0 | 0 | 22.174 | 0 |
| CTONG2008506 | 0 | 0 | 0 | 3.339 | 0 | 2.844 | 0 |
| CTONG2008689 | 0 | 0 | 0 | 14.832 | 0 | 0 | 0 |
| CTONG2008720 | 0 | 0 | 0 | 0 | 0 | 27.203 | 0 |
| CTONG2009534 | 0 | 0 | 0 | 16.375 | 0 | 0 | 0 |
| CTONG2010348 | 0 | 0 | 0 | 7.541 | 0 | 1.784 | 0 |
| CTONG2010408 | 0 | 0 | 0 | 12.163 | 0 | 0 | 0 |
| CTONG2010803 | 0 | 79.029 | 0 | 0 | 0 | 0 | 0 |
| CTONG2010821 | 0 | 0 | 0 | 3.550 | 0 | 1.440 | 0 |
| CTONG2011825 | 0 | 0 | 0 | 6.093 | 0 | 0 | 0 |
| CTONG2012001 | 0 | 0 | 0 | 0 | 0 | 14.410 | 0 |
| CTONG2012101 | 0 | 0 | 0 | 0 | 0 | 15.907 | 0 |
| CTONG2012401 | 0 | 0 | 0 | 0 | 0 | 4.239 | 0 |
| CTONG2012452 | 0 | 2.499 | 2.746 | 1.516 | 3.860 | 1.435 | 0 |
| CTONG2012453 | 0 | 0 | 0 | 5.345 | 0 | 0 | 0 |
| CTONG2012745 | 0 | 38.932 | 0 | 0 | 0 | 0 | 0 |
| CTONG2012847 | 0 | 0 | 0 | 1.222 | 0 | 0 | 0 |
| CTONG2012879 | 0 | 11.934 | 0 | 0 | 0 | 0 | 0 |
| CTONG2013348 | 0 | 0 | 0 | 0 | 0 | 5.543 | 0 |
| CTONG2013907 | 0 | 0 | 0 | 4.591 | 0 | 2.172 | 0 |
| CTONG2013934 | 0 | 0 | 0 | 24.615 | 0 | 0 | 0 |
| CTONG2014280 | 0 | 0 | 0 | 0 | 0 | 25.331 | 0 |
| CTONG2014369 | 0 | 0 | 0 | 2.954 | 0 | 0 | 0 |
| CTONG2014898 | 0 | 0 | 0 | 4.094 | 0 | 0 | 0 |
| CTONG2015091 | 0 | 0 | 0 | 0.576 | 0 | 0 | 0 |
| CTONG2015815 | 0 | 0 | 0 | 6.244 | 0 | 0 | 0 |
| CTONG2016185 | 0 | 0 | 0 | 3.690 | 0 | 2.619 | 0 |
| CTONG2016575 | 0 | 0 | 0 | 1.059 | 0 | 3.507 | 0 |
| CTONG2016824 | 0 | 0 | 0 | 3.021 | 0 | 0 | 0 |
| CTONG2016869 | 0 | 0 | 0 | 5.624 | 0 | 2.661 | 0 |
| CTONG2016931 | 0 | 0 | 0 | 2.166 | 0 | 0 | 0 |
| CTONG2017094 | 0 | 0 | 0 | 9.521 | 0 | 0 | 0 |
| CTONG2017500 | 0 | 0 | 0 | 0 | 0 | 2.669 | 0 |
| CTONG2017998 | 0 | 35.166 | 0 | 3.556 | 0 | 0 | 0 |
| CTONG2018211 | 0 | 0 | 0 | 14.524 | 0 | 0 | 0 |
| CTONG2018343 | 0 | 0 | 0 | 0 | 0 | 15.348 | 0 |
| CTONG2018632 | 0 | 0 | 0 | 5.677 | 0 | 0 | 0 |
| CTONG2018637 | 0 | 0 | 0 | 13.969 | 0 | 0 | 0 |
| CTONG2018739 | 0 | 0 | 0 | 24.755 | 0 | 3.905 | 0 |
| CTONG2018843 | 0 | 0 | 0 | 0 | 0 | 11.186 | 0 |
| CTONG2018898 | 0 | 0 | 0 | 0.684 | 0 | 5.338 | 0 |
| CTONG2019029 | 0 | 0 | 0 | 0 | 0 | 8.154 | 0 |
| CTONG2020374 | 0 | 0 | 0 | 11.030 | 0 | 0 | 0 |
| CTONG2020484 | 0 | 0 | 0 | 0 | 2.124 | 5.923 | 0 |
| CTONG2020582 | 0 | 0 | 0 | 1.362 | 0 | 3.867 | 0 |
| CTONG2021289 | 0 | 0 | 0 | 0 | 0 | 10.367 | 0 |
| CTONG2022835 | 0 | 0 | 0 | 1.218 | 0 | 0 | 0 |
| CTONG2024031 | 0 | 0 | 0 | 3.669 | 0 | 0 | 0 |
| CTONG2027263 | 0 | 0 | 0 | 11.688 | 0 | 0 | 0 |
| CTONG2027959 | 0 | 0 | 0 | 27.592 | 0 | 0 | 0 |
| CTONG2028124 | 0 | 0 | 0 | 2.854 | 0 | 0.506 | 0 |
| CTONG2028208 | 0 | 0 | 0 | 0 | 12.795 | 0 | 0 |
| CTONG2028309 | 0 | 0 | 0 | 10.419 | 0 | 0 | 0 |
| CTONG3000657 | 0 | 0 | 0 | 5.508 | 0 | 0 | 0 |
| CTONG3001746 | 0 | 0 | 0 | 0 | 0 | 14.414 | 0 |
| CTONG3002835 | 0 | 7.711 | 0 | 1.560 | 0 | 1.107 | 0 |
| CTONG3005813 | 0 | 0 | 0 | 27.592 | 0 | 0 | 0 |
| CTONG3008894 | 0 | 0 | 0 | 1.032 | 0 | 4.395 | 0 |
| CTONG3009239 | 0 | 0 | 0 | 0 | 0 | 4.364 | 0 |
| CTONG3009328 | 0 | 0 | 0 | 0 | 43.334 | 0 | 0 |
| D30ST1000270 | 0 | 0 | 0 | 0 | 0 | 7.810 | 0 |
| D60ST2000358 | 0 | 0 | 0 | 0 | 0 | 1.432 | 0 |
| D60ST2000464 | 0 | 0 | 0 | 0 | 0 | 2.208 | 0 |
| D90ST2002608 | 0 | 0 | 0 | 4.744 | 0 | 0 | 0 |
| D90ST2003108 | 0 | 34.233 | 0 | 0 | 0 | 0 | 0 |
| D90ST2003580 | 0 | 0 | 0 | 0 | 0 | 6.953 | 0 |
| DFNES1000083 | 0 | 0 | 0 | 0 | 8.128 | 0 | 0 |
| DFNES2000212 | 0 | 0 | 0 | 0 | 0 | 14.601 | 0 |
| DFNES2000268 | 0 | 7.750 | 0 | 0 | 0 | 0 | 0 |
| DFNES2000335 | 0 | 0 | 0 | 3.363 | 0 | 0 | 0 |
| DFNES2000457 | 0 | 0 | 0 | 6.304 | 0 | 0 | 0 |
| DFNES2000526 | 0 | 0 | 0 | 1.961 | 0 | 2.785 | 0 |
| DFNES2001096 | 0 | 0 | 0 | 0 | 27.690 | 0 | 0 |
| DFNES2001829 | 0 | 0 | 0 | 6.550 | 0 | 0 | 0 |
| DFNES2003192 | 0 | 0 | 0 | 0 | 0 | 3.018 | 0 |
| DFNES2004371 | 0 | 0 | 0 | 1.120 | 0 | 1.590 | 0 |
| DFNES2005540 | 0 | 0 | 23.312 | 0 | 0 | 0 | 0 |
| DFNES2005690 | 0 | 0 | 0 | 3.603 | 0 | 10.231 | 0 |
| DFNES2007634 | 0 | 0 | 0 | 2.523 | 0 | 6.269 | 0 |
| DFNES2008088 | 0 | 0 | 20.274 | 0 | 0 | 0 | 0 |
| DFNES2008160 | 0 | 0 | 0 | 19.078 | 0 | 4.514 | 0 |
| DFNES2009482 | 0 | 0 | 0 | 1.007 | 0 | 1.429 | 0 |
| DFNES2011380 | 0 | 0 | 0 | 0 | 0 | 11.778 | 0 |
| DFNES2011499 | 0 | 0 | 0 | 4.403 | 0 | 0 | 0 |
| FCBBF1000024 | 0 | 0 | 0 | 0 | 0 | 6.637 | 0 |
| FCBBF1000027 | 0 | 0 | 0 | 0.520 | 1.324 | 1.845 | 0 |
| FCBBF1000053 | 0 | 0 | 0 | 0 | 0 | 2.727 | 0 |
| FCBBF1000063 | 0 | 0 | 0 | 0 | 0 | 6.026 | 0 |
| FCBBF1000077 | 0 | 0 | 0 | 0 | 0 | 0.816 | 0 |
| FCBBF1000115 | 0 | 0 | 0 | 14.427 | 0 | 0 | 0 |
| FCBBF1000121 | 0 | 0 | 0 | 0 | 0 | 10.010 | 0 |
| FCBBF1000125 | 0 | 0 | 0 | 11.056 | 0 | 31.390 | 0 |
| FCBBF1000139 | 0 | 0 | 0 | 3.722 | 0 | 1.761 | 0 |
| FCBBF1000197 | 0 | 0 | 0 | 3.033 | 0 | 4.305 | 0 |
| FCBBF1000280 | 0 | 0 | 43.560 | 0 | 0 | 0 | 0 |
| FCBBF1000294 | 0 | 0 | 8.303 | 5.349 | 0 | 1.085 | 0 |
| FCBBF1000367 | 0 | 0 | 0 | 0 | 0 | 1.531 | 0 |
| FCBBF1000403 | 0 | 0 | 0 | 0 | 0 | 8.451 | 0 |
| FCBBF1000466 | 0 | 0 | 0 | 3.164 | 0 | 0.642 | 0 |
| FCBBF1000476 | 0 | 0 | 0 | 6.164 | 0 | 0 | 0 |
| FCBBF1000509 | 0 | 0 | 0 | 0 | 0 | 13.160 | 0 |
| FCBBF1000556 | 0 | 0 | 0 | 8.323 | 3.852 | 4.296 | 0 |
| FCBBF1000563 | 0 | 0 | 0 | 2.132 | 0 | 4.540 | 0 |
| FCBBF1000574 | 0 | 0 | 0 | 2.947 | 0 | 10.458 | 0 |
| FCBBF1000671 | 0 | 0 | 0 | 9.717 | 0 | 0 | 0 |
| FCBBF1000675 | 0 | 0 | 0 | 9.013 | 0 | 12.795 | 0 |
| FCBBF1000687 | 0 | 0 | 0 | 18.394 | 0 | 8.704 | 0 |
| FCBBF1000732 | 0 | 19.654 | 0 | 1.987 | 0 | 0 | 0 |
| FCBBF1000760 | 0 | 4.596 | 0 | 0.465 | 0 | 0 | 0 |
| FCBBF2000038 | 0 | 0 | 0 | 0.953 | 9.707 | 2.707 | 0 |
| FCBBF2000199 | 0 | 0 | 0 | 3.971 | 0 | 2.819 | 0 |
| FCBBF2000362 | 0 | 0 | 0 | 11.882 | 0 | 16.868 | 0 |
| FCBBF2000566 | 0 | 0 | 0 | 0 | 0 | 3.745 | 0 |
| FCBBF2000733 | 0 | 0 | 0 | 0 | 0 | 7.986 | 0 |
| FCBBF2000885 | 0 | 0 | 0 | 0 | 0 | 3.093 | 0 |
| FCBBF2001183 | 0 | 0 | 0 | 5.869 | 5.975 | 9.998 | 0 |
| FCBBF2001211 | 0 | 0 | 0 | 0 | 0 | 6.457 | 0 |
| FCBBF2001291 | 0 | 0 | 0 | 2.701 | 0 | 0 | 0 |
| FCBBF2001299 | 0 | 0 | 0 | 16.114 | 0 | 0 | 0 |
| FCBBF2001538 | 0 | 0 | 0 | 4.326 | 0 | 4.606 | 0 |
| FCBBF2001594 | 0 | 0 | 0 | 21.593 | 0 | 7.664 | 0 |
| FCBBF2002349 | 0 | 0 | 0 | 2.802 | 0 | 0 | 0 |
| FCBBF2002626 | 0 | 0 | 0 | 27.755 | 0 | 0 | 0 |
| FCBBF2002801 | 0 | 0 | 0 | 1.842 | 0 | 2.615 | 0 |
| FCBBF2003567 | 0 | 0 | 0 | 0 | 23.554 | 13.136 | 0 |
| FCBBF2004066 | 0 | 0 | 0 | 5.723 | 21.853 | 6.094 | 0 |
| FCBBF2005428 | 0 | 0 | 0 | 27.755 | 0 | 0 | 0 |
| FCBBF2005909 | 0 | 0 | 0 | 4.430 | 0 | 0 | 0 |
| FCBBF2005966 | 0 | 0 | 0 | 13.444 | 0 | 0 | 0 |
| FCBBF2006781 | 0 | 44.415 | 0 | 0 | 0 | 6.376 | 0 |
| FCBBF2007556 | 0 | 64.144 | 0 | 0 | 0 | 0 | 0 |
| FCBBF2007840 | 0 | 0 | 0 | 8.840 | 0 | 0 | 0 |
| FCBBF3000115 | 0 | 0 | 0 | 14.163 | 0 | 4.021 | 0 |
| FCBBF3000168 | 0 | 0 | 0 | 5.779 | 0 | 0 | 0 |
| FCBBF3000175 | 0 | 0 | 0 | 0 | 15.172 | 12.693 | 0 |
| FCBBF3000228 | 0 | 0 | 0 | 6.361 | 0 | 0 | 0 |
| FCBBF3000269 | 0 | 0 | 0 | 43.451 | 0 | 0 | 0 |
| FCBBF3000388 | 0 | 0 | 0 | 0 | 0 | 2.489 | 0 |
| FCBBF3000434 | 0 | 0 | 0 | 14.454 | 6.690 | 3.731 | 0 |
| FCBBF3001081 | 0 | 0 | 0 | 1.864 | 9.488 | 2.646 | 0 |
| FCBBF3001470 | 0 | 9.528 | 0 | 0 | 0 | 0 | 0 |
| FCBBF3001632 | 0 | 0 | 0 | 1.096 | 0 | 0 | 0 |
| FCBBF3001657 | 0 | 0 | 0 | 11.720 | 0 | 4.754 | 0 |
| FCBBF3001818 | 0 | 0 | 0 | 6.465 | 0 | 0 | 0 |
| FCBBF3001855 | 0 | 0 | 0 | 1.988 | 0 | 0 | 0 |
| FCBBF3001977 | 0 | 0 | 0 | 10.573 | 0 | 15.010 | 0 |
| FCBBF3003800 | 0 | 0 | 0 | 5.871 | 0 | 1.985 | 0 |
| FCBBF3004323 | 0 | 0 | 0 | 0 | 0 | 5.551 | 0 |
| FCBBF3004473 | 0 | 0 | 0 | 0 | 0 | 1.158 | 0 |
| FCBBF3005330 | 0 | 0 | 0 | 2.458 | 0 | 0 | 0 |
| FCBBF3006821 | 0 | 0 | 0 | 8.115 | 0 | 5.760 | 0 |
| FCBBF3007631 | 0 | 0 | 0 | 15.506 | 0 | 0 | 0 |
| FCBBF3007859 | 0 | 0 | 0 | 13.832 | 0 | 13.091 | 0 |
| FCBBF3007860 | 0 | 0 | 0 | 0 | 0 | 20.391 | 0 |
| FCBBF3008100 | 0 | 0 | 0 | 0 | 53.906 | 0 | 0 |
| FCBBF3008153 | 0 | 0 | 0 | 7.766 | 0 | 5.513 | 0 |
| FCBBF3008689 | 0 | 65.513 | 0 | 0 | 0 | 0 | 0 |
| FCBBF3009069 | 0 | 0 | 0 | 6.357 | 0 | 22.561 | 0 |
| FCBBF3009526 | 0 | 0 | 0 | 0 | 0 | 23.808 | 0 |
| FCBBF3009541 | 0 | 2.893 | 0 | 0.293 | 0 | 0.415 | 0 |
| FCBBF3010012 | 0 | 0 | 0 | 4.893 | 8.303 | 0 | 0 |
| FCBBF3010142 | 0 | 0 | 0 | 10.244 | 0 | 0 | 0 |
| FCBBF3010508 | 0 | 79.163 | 0 | 0 | 0 | 0 | 0 |
| FCBBF3010695 | 0 | 0 | 0 | 1.760 | 0 | 0 | 0 |
| FCBBF3012182 | 0 | 0 | 0 | 0 | 0 | 0 | 99.513 |
| FCBBF3012667 | 0 | 0 | 0 | 0 | 0 | 24.592 | 0 |
| FCBBF3013506 | 0 | 0 | 0 | 16.855 | 0 | 0 | 0 |
| FCBBF3014260 | 0 | 0 | 0 | 0 | 0 | 6.825 | 0 |
| FCBBF3015100 | 0 | 0 | 0 | 14.166 | 0 | 5.028 | 0 |
| FCBBF3016928 | 0 | 0 | 0 | 1.866 | 3.166 | 1.766 | 0 |
| FCBBF3017396 | 0 | 0 | 0 | 4.347 | 0 | 2.057 | 0 |
| FCBBF3017531 | 0 | 22.507 | 0 | 0 | 5.793 | 0 | 0 |
| FCBBF3017918 | 0 | 0 | 0 | 4.647 | 0 | 0 | 0 |
| FCBBF3018453 | 0 | 0 | 0 | 3.279 | 0 | 0 | 0 |
| FCBBF3018796 | 0 | 0 | 0 | 2.203 | 0 | 0 | 0 |
| FCBBF3018826 | 0 | 0 | 0 | 0 | 0 | 9.829 | 0 |
| FCBBF3019320 | 0 | 0 | 0 | 14.030 | 0 | 0 | 0 |
| FCBBF3019569 | 0 | 0 | 0 | 27.755 | 0 | 0 | 0 |
| FCBBF3019714 | 0 | 0 | 0 | 0 | 7.401 | 0 | 0 |
| FCBBF3019839 | 0 | 0 | 0 | 5.316 | 0 | 3.773 | 0 |
| FCBBF3021807 | 0 | 0 | 0 | 0 | 31.333 | 0 | 0 |
| FCBBF3022291 | 0 | 0 | 0 | 27.755 | 0 | 0 | 0 |
| FCBBF3022311 | 0 | 0 | 0 | 16.114 | 0 | 0 | 0 |
| FCBBF3022593 | 0 | 0 | 0 | 0 | 0 | 6.981 | 0 |
| FCBBF3022765 | 0 | 0 | 0 | 0 | 0 | 35.292 | 0 |
| FCBBF3023667 | 0 | 0 | 7.169 | 0.189 | 0 | 1.338 | 0 |
| FCBBF3024002 | 0 | 0 | 0 | 16.112 | 0 | 5.718 | 0 |
| FCBBF3024266 | 0 | 0 | 0 | 0 | 0 | 19.712 | 0 |
| FCBBF3024793 | 0 | 0 | 0 | 11.340 | 0 | 0 | 0 |
| FCBBF3025417 | 0 | 0 | 0 | 0 | 0 | 17.298 | 0 |
| FCBBF3025905 | 0 | 0 | 0 | 11.354 | 0 | 0 | 0 |
| FCBBF3026651 | 0 | 0 | 0 | 5.179 | 0 | 0 | 0 |
| FCBBF3026692 | 0 | 0 | 0 | 11.517 | 0 | 0 | 0 |
| FCBBF3028244 | 0 | 0 | 0 | 3.295 | 3.355 | 3.742 | 0 |
| FCBBF4000017 | 0 | 0 | 0 | 0 | 0 | 8.949 | 0 |
| FCBBF4000142 | 0 | 0 | 0 | 18.164 | 0 | 11.051 | 0 |
| FCBBF4000446 | 0 | 0 | 5.793 | 0 | 0 | 3.027 | 0 |
| FCBBF4000548 | 0 | 0 | 0 | 10.374 | 0 | 14.727 | 0 |
| FCBBF5000165 | 0 | 0 | 0 | 11.682 | 0 | 16.583 | 0 |
| FCBBF5000325 | 0 | 0 | 0 | 0 | 0 | 10.186 | 0 |
| FCBBF5000353 | 0 | 0 | 0 | 0 | 0 | 35.292 | 0 |
| FEBRA1000022 | 0 | 0 | 0 | 11.207 | 0 | 0 | 0 |
| FEBRA1000057 | 0 | 0 | 0 | 4.929 | 6.273 | 0 | 0 |
| FEBRA1000088 | 0 | 0 | 0 | 0 | 0 | 4.300 | 0 |
| FEBRA1000188 | 0 | 0 | 0 | 0 | 0 | 2.109 | 0 |
| FEBRA1000189 | 0 | 0 | 0 | 28.778 | 0 | 0 | 0 |
| FEBRA2000007 | 0 | 0 | 0 | 16.808 | 0 | 0 | 0 |
| FEBRA2000105 | 0 | 0 | 0 | 0.509 | 0 | 0.723 | 0 |
| FEBRA2000129 | 0 | 0 | 0 | 0 | 9.393 | 0 | 0 |
| FEBRA2000210 | 0 | 4.962 | 0 | 0 | 0 | 0 | 0 |
| FEBRA2000377 | 0 | 0 | 0 | 7.100 | 0 | 10.080 | 0 |
| FEBRA2000402 | 0 | 0 | 0 | 0 | 0 | 5.585 | 0 |
| FEBRA2000404 | 0 | 0 | 0 | 7.141 | 0 | 0 | 0 |
| FEBRA2000438 | 0 | 0 | 0 | 6.271 | 0 | 0 | 0 |
| FEBRA2000452 | 0 | 0 | 0 | 4.271 | 0 | 2.021 | 0 |
| FEBRA2000462 | 0 | 0 | 0 | 28.049 | 0 | 0 | 0 |
| FEBRA2000536 | 0 | 0 | 0 | 13.414 | 0 | 15.869 | 0 |
| FEBRA2000581 | 0 | 0 | 0 | 1.694 | 0 | 13.626 | 0 |
| FEBRA2000680 | 0 | 0 | 0 | 33.354 | 0 | 0 | 0 |
| FEBRA2000733 | 5.282 | 2.366 | 0 | 1.316 | 1.218 | 0.340 | 0 |
| FEBRA2000740 | 0 | 7.938 | 0 | 2.408 | 0 | 1.140 | 0 |
| FEBRA2000757 | 0 | 0 | 0 | 0 | 0 | 9.257 | 0 |
| FEBRA2000771 | 0 | 0 | 0 | 0 | 0 | 21.859 | 0 |
| FEBRA2000782 | 0 | 0 | 0 | 36.283 | 0 | 0 | 0 |
| FEBRA2000787 | 0 | 0 | 0 | 2.804 | 0 | 5.971 | 0 |
| FEBRA2000793 | 0 | 0 | 0 | 2.722 | 0 | 0 | 0 |
| FEBRA2000815 | 0 | 0 | 0 | 5.132 | 0 | 3.643 | 0 |
| FEBRA2000877 | 0 | 0 | 0 | 20.195 | 0 | 14.335 | 0 |
| FEBRA2000880 | 0 | 0 | 0 | 7.521 | 0 | 10.677 | 0 |
| FEBRA2000881 | 0 | 0 | 0 | 2.247 | 0 | 0 | 0 |
| FEBRA2000972 | 0 | 0 | 0 | 10.751 | 0 | 0 | 0 |
| FEBRA2001004 | 0 | 0 | 0 | 0 | 0 | 22.288 | 0 |
| FEBRA2001146 | 0 | 0 | 0 | 15.874 | 0 | 0 | 0 |
| FEBRA2001197 | 0 | 0 | 0 | 0 | 0 | 14.203 | 0 |
| FEBRA2001245 | 0 | 0 | 0 | 3.581 | 0 | 10.166 | 0 |
| FEBRA2001294 | 0 | 0 | 0 | 0 | 0 | 2.435 | 0 |
| FEBRA2001334 | 0 | 0 | 0 | 24.490 | 20.779 | 0 | 0 |
| FEBRA2001383 | 0 | 0 | 0 | 7.404 | 0 | 10.511 | 0 |
| FEBRA2001492 | 0 | 0 | 0 | 6.892 | 0 | 3.261 | 0 |
| FEBRA2001571 | 0 | 0 | 0 | 4.465 | 0 | 4.226 | 0 |
| FEBRA2001669 | 0 | 0 | 0 | 2.281 | 0 | 6.476 | 0 |
| FEBRA2001715 | 0 | 0 | 0 | 9.908 | 0 | 4.019 | 0 |
| FEBRA2001745 | 0 | 0 | 0 | 4.447 | 0 | 0 | 0 |
| FEBRA2001752 | 0 | 0 | 0 | 11.179 | 0 | 0 | 0 |
| FEBRA2001814 | 0 | 0 | 0 | 7.512 | 14.341 | 14.663 | 0 |
| FEBRA2001828 | 0 | 0 | 0 | 3.861 | 0 | 0 | 0 |
| FEBRA2001974 | 0 | 0 | 0 | 0 | 0 | 11.740 | 0 |
| FEBRA2001990 | 0 | 0 | 0 | 15.453 | 0 | 7.313 | 0 |
| FEBRA2002027 | 0 | 0 | 0 | 1.798 | 0 | 17.869 | 0 |
| FEBRA2002191 | 0 | 0 | 0 | 0 | 0 | 5.919 | 0 |
| FEBRA2002352 | 0 | 0 | 0 | 4.928 | 0 | 3.498 | 0 |
| FEBRA2002456 | 0 | 0 | 0 | 3.871 | 3.941 | 7.693 | 0 |
| FEBRA2002527 | 0 | 0 | 0 | 0 | 0 | 2.886 | 0 |
| FEBRA2002781 | 0 | 0 | 0 | 43.707 | 0 | 6.894 | 0 |
| FEBRA2002792 | 0 | 0 | 0 | 7.600 | 4.836 | 6.743 | 0 |
| FEBRA2002931 | 0 | 0 | 0 | 1.553 | 0 | 9.924 | 0 |
| FEBRA2002933 | 0 | 0 | 2.854 | 0.263 | 1.337 | 0 | 0 |
| FEBRA2003100 | 0 | 0 | 0 | 0.935 | 0 | 0.664 | 0 |
| FEBRA2003128 | 0 | 0 | 0 | 6.194 | 0 | 2.931 | 0 |
| FEBRA2003258 | 0 | 0 | 0 | 2.008 | 10.223 | 0 | 0 |
| FEBRA2003436 | 0 | 0 | 0 | 0 | 0 | 5.601 | 0 |
| FEBRA2003470 | 0 | 0 | 0 | 0 | 0 | 14.706 | 0 |
| FEBRA2003541 | 0 | 0 | 0 | 0 | 0 | 3.075 | 0 |
| FEBRA2003726 | 0 | 0 | 0 | 0.707 | 3.598 | 1.003 | 0 |
| FEBRA2003750 | 0 | 0 | 0 | 2.591 | 0 | 1.839 | 0 |
| FEBRA2003833 | 0 | 0 | 0 | 7.364 | 0 | 1.742 | 0 |
| FEBRA2003907 | 0 | 0 | 0 | 22.162 | 0 | 0 | 0 |
| FEBRA2003926 | 0 | 0 | 0 | 0.495 | 0 | 1.407 | 0 |
| FEBRA2004042 | 0 | 0 | 0 | 1.506 | 0 | 2.137 | 0 |
| FEBRA2004091 | 0 | 0 | 0 | 15.001 | 0 | 10.648 | 0 |
| FEBRA2004224 | 0 | 0 | 0 | 1.643 | 4.183 | 1.167 | 0 |
| FEBRA2004237 | 0 | 0 | 0 | 53.246 | 0 | 0 | 0 |
| FEBRA2004268 | 0 | 0 | 0 | 12.462 | 0 | 0 | 0 |
| FEBRA2004293 | 0 | 0 | 0 | 1.887 | 0 | 2.679 | 0 |
| FEBRA2004412 | 0 | 0 | 0 | 2.084 | 0 | 0 | 0 |
| FEBRA2004485 | 0 | 0 | 0 | 0 | 25.077 | 6.993 | 0 |
| FEBRA2004620 | 0 | 0 | 75.571 | 0 | 0 | 0 | 0 |
| FEBRA2004628 | 0 | 0 | 0 | 6.074 | 0 | 2.874 | 0 |
| FEBRA2004767 | 0 | 0 | 0 | 36.283 | 0 | 0 | 0 |
| FEBRA2004818 | 0 | 0 | 0 | 6.765 | 4.920 | 10.976 | 0 |
| FEBRA2005079 | 0 | 0 | 0 | 0 | 0 | 16.812 | 0 |
| FEBRA2005114 | 0 | 0 | 0 | 9.525 | 0 | 0 | 0 |
| FEBRA2005361 | 0 | 0 | 0 | 0.497 | 7.592 | 13.408 | 0 |
| FEBRA2005416 | 33.661 | 0 | 0 | 0 | 0 | 0 | 0 |
| FEBRA2005476 | 0 | 0 | 0 | 0 | 0 | 2.624 | 0 |
| FEBRA2005572 | 0 | 0 | 0 | 2.529 | 0 | 0 | 0 |
| FEBRA2005734 | 0 | 0 | 0 | 1.798 | 0 | 0 | 0 |
| FEBRA2005752 | 0 | 0 | 0 | 2.954 | 15.040 | 4.194 | 0 |
| FEBRA2005788 | 0 | 0 | 0 | 0 | 0 | 4.973 | 0 |
| FEBRA2005824 | 0 | 0 | 0 | 1.588 | 16.173 | 4.886 | 0 |
| FEBRA2006092 | 0 | 0 | 0 | 2.648 | 2.247 | 1.880 | 0 |
| FEBRA2006112 | 0 | 0 | 0 | 4.685 | 0 | 13.301 | 0 |
| FEBRA2006255 | 0 | 0 | 0 | 0 | 0 | 5.252 | 0 |
| FEBRA2006270 | 0 | 0 | 0 | 0.830 | 0 | 1.178 | 0 |
| FEBRA2006346 | 0 | 0 | 0 | 10.544 | 0 | 0 | 0 |
| FEBRA2006357 | 0 | 0 | 0 | 13.633 | 0 | 12.680 | 0 |
| FEBRA2006368 | 0 | 0 | 0 | 4.411 | 11.228 | 6.262 | 0 |
| FEBRA2006372 | 0 | 0 | 0 | 0.953 | 0 | 1.353 | 0 |
| FEBRA2006396 | 0 | 0 | 0 | 5.705 | 0 | 0 | 0 |
| FEBRA2006401 | 0 | 0 | 0 | 0 | 0 | 0.759 | 0 |
| FEBRA2006485 | 0 | 0 | 0 | 0 | 0 | 3.065 | 0 |
| FEBRA2006519 | 0 | 0 | 0 | 25.320 | 0 | 11.982 | 0 |
| FEBRA2006521 | 0 | 0 | 0 | 10.857 | 0 | 7.706 | 0 |
| FEBRA2006553 | 0 | 0 | 0 | 2.808 | 0 | 0 | 0 |
| FEBRA2006736 | 0 | 0 | 0 | 10.544 | 0 | 0 | 0 |
| FEBRA2006808 | 0 | 0 | 0 | 0 | 0 | 8.272 | 0 |
| FEBRA2006873 | 0 | 0 | 34.629 | 0 | 0 | 0 | 0 |
| FEBRA2007017 | 0 | 0 | 0 | 0 | 59.175 | 0 | 0 |
| FEBRA2007212 | 0 | 0 | 0 | 11.144 | 0 | 0 | 0 |
| FEBRA2007534 | 0 | 0 | 0 | 0 | 0 | 18.185 | 0 |
| FEBRA2007544 | 0 | 0 | 0 | 41.703 | 0 | 0 | 0 |
| FEBRA2007589 | 0 | 0 | 0 | 19.411 | 0 | 13.778 | 0 |
| FEBRA2007613 | 0 | 0 | 0 | 4.711 | 0 | 3.344 | 0 |
| FEBRA2007622 | 0 | 0 | 0 | 0 | 0 | 1.040 | 0 |
| FEBRA2007708 | 0 | 0 | 0 | 6.066 | 0 | 0 | 0 |
| FEBRA2007714 | 0 | 0 | 0 | 5.922 | 0 | 16.815 | 0 |
| FEBRA2007801 | 0 | 0 | 0 | 1.752 | 0 | 0 | 0 |
| FEBRA2007880 | 0 | 0 | 0 | 0 | 0 | 3.792 | 0 |
| FEBRA2007900 | 0 | 0 | 0 | 11.003 | 0 | 0 | 0 |
| FEBRA2007901 | 0 | 0 | 0 | 0 | 0 | 3.734 | 0 |
| FEBRA2008036 | 0 | 0 | 0 | 9.124 | 0 | 4.318 | 0 |
| FEBRA2008081 | 0 | 0 | 0 | 1.508 | 0 | 2.141 | 0 |
| FEBRA2008087 | 0 | 1.258 | 1.382 | 0.382 | 0 | 0.181 | 0 |
| FEBRA2008134 | 0 | 0 | 0 | 0 | 0 | 28.785 | 0 |
| FEBRA2008159 | 0 | 0 | 0 | 22.162 | 0 | 0 | 0 |
| FEBRA2008201 | 0 | 16.832 | 0 | 0 | 0 | 0 | 0 |
| FEBRA2008210 | 0 | 0 | 0 | 4.761 | 0 | 6.758 | 0 |
| FEBRA2008255 | 0 | 0 | 0 | 7.183 | 18.284 | 5.099 | 0 |
| FEBRA2008265 | 0 | 0 | 0 | 0 | 59.175 | 0 | 0 |
| FEBRA2008287 | 0 | 0 | 0 | 4.184 | 0 | 2.970 | 0 |
| FEBRA2008302 | 0 | 7.470 | 0 | 0 | 3.845 | 3.217 | 0 |
| FEBRA2008459 | 0 | 0 | 0 | 0 | 0 | 28.785 | 0 |
| FEBRA2008583 | 0 | 0 | 0 | 1.313 | 3.821 | 1.332 | 0 |
| FEBRA2008662 | 0 | 0 | 0 | 5.633 | 0 | 6.397 | 0 |
| FEBRA2008859 | 0 | 0 | 0 | 22.162 | 0 | 0 | 0 |
| FEBRA2008861 | 0 | 0 | 0 | 4.147 | 0 | 3.925 | 0 |
| FEBRA2008928 | 0 | 0 | 0 | 22.162 | 0 | 0 | 0 |
| FEBRA2009022 | 0 | 0 | 0 | 7.111 | 0 | 3.365 | 0 |
| FEBRA2009185 | 0 | 0 | 0 | 12.897 | 0 | 26.548 | 0 |
| FEBRA2009289 | 0 | 0 | 0 | 0 | 50.703 | 0 | 0 |
| FEBRA2009328 | 0 | 0 | 0 | 3.517 | 0 | 0 | 0 |
| FEBRA2009419 | 0 | 0 | 0 | 3.751 | 0 | 3.994 | 0 |
| FEBRA2009514 | 0 | 0 | 0 | 0 | 0 | 3.338 | 0 |
| FEBRA2010719 | 0 | 0 | 0 | 0 | 0 | 17.484 | 0 |
| FEBRA2010746 | 0 | 0 | 0 | 6.089 | 0 | 0 | 0 |
| FEBRA2012181 | 0 | 0 | 0 | 11.719 | 0 | 0 | 0 |
| FEBRA2012507 | 0 | 0 | 0 | 0 | 26.964 | 0 | 0 |
| FEBRA2013699 | 0 | 0 | 0 | 0 | 0 | 7.211 | 0 |
| FEBRA2014297 | 0 | 0 | 0 | 0 | 7.723 | 4.307 | 0 |
| FEBRA2014417 | 0 | 0 | 0 | 0.766 | 0 | 3.264 | 0 |
| FEBRA2014939 | 0 | 0 | 0 | 11.858 | 0 | 0 | 0 |
| FEBRA2015085 | 0 | 0 | 0 | 2.164 | 0 | 7.681 | 0 |
| FEBRA2017024 | 0 | 0 | 0 | 6.848 | 0 | 0 | 0 |
| FEBRA2017533 | 0 | 0 | 0 | 15.488 | 0 | 5.497 | 0 |
| FEBRA2017680 | 0 | 0 | 0 | 0 | 0 | 9.581 | 0 |
| FEBRA2018203 | 0 | 0 | 0 | 0 | 0 | 15.289 | 0 |
| FEBRA2018433 | 0 | 0 | 0 | 22.162 | 0 | 0 | 0 |
| FEBRA2018476 | 0 | 7.669 | 0 | 0 | 0 | 1.101 | 0 |
| FEBRA2018746 | 0 | 0 | 0 | 5.308 | 0 | 0 | 0 |
| FEBRA2019242 | 0 | 0 | 0 | 5.758 | 0 | 0 | 0 |
| FEBRA2019389 | 0 | 0 | 0 | 0 | 59.175 | 0 | 0 |
| FEBRA2021032 | 0 | 0 | 0 | 0 | 0 | 5.402 | 0 |
| FEBRA2021171 | 0 | 0 | 0 | 0 | 0 | 10.719 | 0 |
| FEBRA2021550 | 0 | 0 | 0 | 22.162 | 0 | 0 | 0 |
| FEBRA2021636 | 0 | 0 | 0 | 14.361 | 0 | 0 | 0 |
| FEBRA2021864 | 0 | 0 | 0 | 22.162 | 0 | 0 | 0 |
| FEBRA2021966 | 0 | 0 | 0 | 3.062 | 0 | 0 | 0 |
| FEBRA2022055 | 0 | 0 | 0 | 0.378 | 0 | 0 | 0 |
| FEBRA2022601 | 0 | 0 | 0 | 22.162 | 0 | 0 | 0 |
| FEBRA2023377 | 0 | 0 | 0 | 0 | 28.030 | 0 | 0 |
| FEBRA2023399 | 0 | 0 | 0 | 6.230 | 0 | 0 | 0 |
| FEBRA2023550 | 0 | 0 | 0 | 0 | 59.175 | 0 | 0 |
| FEBRA2023927 | 0 | 0 | 0 | 8.672 | 0 | 0 | 0 |
| FEBRA2024019 | 0 | 0 | 0 | 0 | 0 | 8.107 | 0 |
| FEBRA2024987 | 0 | 69.047 | 0 | 0 | 0 | 0 | 0 |
| FEBRA2025427 | 0 | 0 | 0 | 0 | 0 | 4.995 | 0 |
| FEBRA2025838 | 0 | 0 | 0 | 0 | 0 | 1.655 | 0 |
| FEBRA2026984 | 0 | 0 | 0 | 12.446 | 0 | 0 | 0 |
| FEBRA2028503 | 0 | 0 | 0 | 0 | 0 | 14.089 | 0 |
| FEBRA2028516 | 0 | 0 | 0 | 1.624 | 0 | 2.305 | 0 |
| FELIV1000137 | 0 | 0 | 0 | 0.220 | 0 | 0 | 0 |
| FELIV1000153 | 5.170 | 0 | 0 | 0.234 | 0 | 0.166 | 0 |
| HCASM1000115 | 0 | 0 | 0 | 1.471 | 0 | 2.089 | 0 |
| HCASM2000016 | 0 | 15.716 | 0 | 0 | 0 | 1.504 | 0 |
| HCASM2000138 | 0 | 0 | 0 | 8.993 | 0 | 4.256 | 0 |
| HCASM2000156 | 0 | 0 | 0 | 0 | 0 | 13.176 | 0 |
| HCASM2000214 | 0 | 0 | 0 | 0 | 0 | 9.530 | 0 |
| HCASM2003415 | 52.578 | 0 | 0 | 0 | 0 | 0 | 0 |
| HCASM2005388 | 0 | 0 | 0 | 2.510 | 0 | 0 | 0 |
| HCHON1000015 | 0 | 0 | 0 | 1.106 | 0 | 1.571 | 0 |
| HCHON1000030 | 0 | 0 | 2.798 | 0.772 | 0 | 1.828 | 0 |
| HCHON1000166 | 0 | 0 | 0 | 0 | 0 | 3.031 | 0 |
| HCHON1000191 | 0 | 0 | 3.725 | 0 | 0 | 0 | 0 |
| HCHON2000056 | 0 | 1.991 | 2.187 | 1.007 | 0 | 1.143 | 0 |
| HCHON2000062 | 0 | 29.418 | 0 | 2.975 | 0 | 0 | 0 |
| HCHON2000160 | 0 | 0 | 0 | 1.429 | 3.637 | 1.014 | 0 |
| HCHON2000226 | 0 | 0 | 0 | 1.431 | 0 | 0 | 0 |
| HCHON2000244 | 0 | 0 | 0 | 0.934 | 0 | 0.663 | 0 |
| HCHON2000265 | 0 | 0 | 0 | 2.872 | 0 | 0 | 0 |
| HCHON2000295 | 0 | 0 | 0 | 0.311 | 0 | 0.883 | 0 |
| HCHON2000323 | 0 | 13.002 | 0 | 0 | 0 | 0 | 0 |
| HCHON2000473 | 0 | 0 | 0 | 8.309 | 0 | 0 | 0 |
| HCHON2000475 | 0 | 0 | 0 | 0 | 0 | 2.682 | 0 |
| HCHON2000508 | 0 | 0 | 0 | 0 | 0 | 0.376 | 0 |
| HCHON2000626 | 0 | 0 | 0 | 0.834 | 0 | 0 | 0 |
| HCHON2000676 | 0 | 0 | 0 | 2.116 | 0 | 0 | 0 |
| HCHON2000698 | 0 | 0 | 0 | 0 | 0 | 5.776 | 0 |
| HCHON2000743 | 0 | 0 | 0 | 8.667 | 0 | 0 | 0 |
| HCHON2000815 | 0 | 0 | 0 | 0 | 0 | 2.814 | 0 |
| HCHON2000819 | 0 | 0 | 0 | 0 | 26.473 | 0 | 0 |
| HCHON2000826 | 0 | 0 | 0 | 1.524 | 0 | 0 | 0 |
| HCHON2001039 | 0 | 0 | 0 | 2.376 | 0 | 0 | 0 |
| HCHON2001050 | 45.433 | 0 | 0 | 0 | 0 | 0 | 0 |
| HCHON2001108 | 0 | 0 | 0 | 1.516 | 0 | 0 | 0 |
| HCHON2001161 | 0 | 0 | 0 | 7.642 | 0 | 10.849 | 0 |
| HCHON2001200 | 0 | 0 | 0 | 0.307 | 0 | 0.872 | 0 |
| HCHON2001217 | 0 | 0 | 0 | 2.216 | 0 | 0 | 0 |
| HCHON2001359 | 0 | 5.976 | 6.566 | 1.813 | 0 | 0.858 | 0 |
| HCHON2001407 | 24.752 | 0 | 0 | 0 | 0 | 0.796 | 0 |
| HCHON2001434 | 0 | 0 | 0 | 4.337 | 5.520 | 4.618 | 0 |
| HCHON2001497 | 0 | 52.590 | 0 | 0 | 0 | 0 | 0 |
| HCHON2001505 | 0 | 0 | 0 | 3.189 | 0 | 0 | 0 |
| HCHON2001665 | 0 | 0 | 0 | 2.191 | 0 | 9.329 | 0 |
| HCHON2001853 | 0 | 0 | 0 | 7.950 | 0 | 0 | 0 |
| HCHON2002247 | 0 | 19.705 | 0 | 1.993 | 10.144 | 0 | 0 |
| HCHON2002354 | 0 | 0 | 0 | 9.017 | 0 | 0 | 0 |
| HCHON2004002 | 0 | 0 | 0 | 0 | 0 | 1.582 | 0 |
| HCHON2005921 | 0 | 0 | 0 | 2.944 | 0 | 0 | 0 |
| HCHON2008112 | 0 | 0 | 0 | 0 | 0 | 13.736 | 0 |
| HCHON2010543 | 0 | 0 | 0 | 6.367 | 0 | 0 | 0 |
| HEART1000010 | 0 | 23.980 | 0 | 0 | 0 | 0 | 0 |
| HEART1000074 | 0 | 5.328 | 5.854 | 1.078 | 0 | 0 | 0 |
| HEART1000149 | 0 | 0 | 0 | 1.333 | 0 | 0 | 0 |
| HEART1000173 | 0 | 0 | 0 | 0 | 0 | 2.645 | 0 |
| HEART2000306 | 0 | 0 | 0 | 9.616 | 0 | 0 | 0 |
| HEART2000506 | 0 | 0 | 0 | 0 | 0 | 1.767 | 0 |
| HEART2000520 | 0 | 0 | 0 | 0 | 0 | 1.453 | 0 |
| HEART2000959 | 0 | 0 | 0 | 8.360 | 0 | 0 | 0 |
| HEART2003852 | 0 | 0 | 0 | 0 | 14.078 | 3.926 | 0 |
| HEART2005310 | 0 | 48.933 | 0 | 0 | 0 | 0 | 0 |
| HEART2006487 | 0 | 0 | 0 | 0 | 35.133 | 0 | 0 |
| HEART2006521 | 0 | 0 | 0 | 3.395 | 0 | 2.410 | 0 |
| HEART2007231 | 0 | 0 | 0 | 0 | 0 | 4.881 | 0 |
| HEART2009000 | 0 | 0 | 0 | 7.336 | 0 | 0 | 0 |
| HHDPC1000083 | 0 | 0 | 0 | 1.389 | 0 | 1.972 | 0 |
| HHDPC1000114 | 0 | 0 | 0 | 1.547 | 0 | 0.732 | 0 |
| HHDPC1000163 | 0 | 0 | 0 | 0 | 0 | 1.267 | 0 |
| HHDPC2000055 | 0 | 0 | 0 | 0 | 0 | 1.842 | 0 |
| HHDPC2000104 | 0 | 0 | 0 | 1.313 | 0 | 0 | 0 |
| HHDPC2000115 | 0 | 0 | 0 | 0 | 0 | 2.636 | 0 |
| HHDPC2000149 | 0 | 0 | 0 | 3.171 | 0 | 0 | 0 |
| HHDPC2000455 | 0 | 0 | 0 | 0.390 | 0 | 0.554 | 0 |
| HHDPC2000456 | 0 | 0 | 0 | 0 | 0 | 1.564 | 0 |
| HHDPC2003439 | 0 | 1.785 | 1.961 | 1.625 | 0 | 0.513 | 0 |
| HHDPC2003983 | 0 | 0 | 2.147 | 0.791 | 1.006 | 0.842 | 0 |
| HHDPC2005794 | 0 | 0 | 0 | 0 | 0 | 8.015 | 0 |
| HHDPC2006862 | 0 | 0 | 0 | 0 | 0 | 7.541 | 0 |
| HHDPC2007267 | 0 | 0 | 0 | 0 | 0 | 0.492 | 0 |
| HHDPC2009528 | 0 | 0 | 0 | 4.613 | 0 | 0 | 0 |
| HLUNG1000055 | 0 | 3.799 | 0 | 0 | 0 | 0 | 0 |
| HLUNG1000064 | 0 | 0 | 0 | 4.833 | 0 | 0 | 0 |
| HLUNG2000232 | 0 | 0 | 0 | 17.890 | 0 | 0 | 0 |
| HLUNG2000314 | 0 | 0 | 0 | 0 | 0 | 2.472 | 0 |
| HLUNG2000412 | 0 | 0 | 0 | 7.602 | 0 | 2.698 | 0 |
| HLUNG2000501 | 0 | 0 | 0 | 0 | 6.199 | 0 | 0 |
| HLUNG2000846 | 0 | 0 | 0 | 0 | 0 | 4.552 | 0 |
| HLUNG2001146 | 0 | 0 | 0 | 3.721 | 0 | 0 | 0 |
| HLUNG2001507 | 0 | 0 | 0 | 0 | 0 | 5.046 | 0 |
| HLUNG2001518 | 0 | 0 | 0 | 5.433 | 0 | 1.102 | 0 |
| HLUNG2001712 | 0 | 0 | 0 | 0 | 35.740 | 0 | 0 |
| HLUNG2002465 | 0 | 0 | 0 | 0 | 0 | 4.875 | 0 |
| HLUNG2002648 | 0 | 0 | 0 | 0 | 0 | 3.642 | 0 |
| HLUNG2002811 | 0 | 0 | 0 | 6.918 | 0 | 4.910 | 0 |
| HLUNG2003378 | 0 | 0 | 0 | 6.571 | 0 | 0 | 0 |
| HLUNG2003716 | 0 | 0 | 0 | 16.214 | 0 | 0 | 0 |
| HLUNG2004067 | 0 | 0 | 0 | 7.815 | 0 | 0 | 0 |
| HLUNG2004159 | 0 | 0 | 0 | 0 | 0 | 1.827 | 0 |
| HLUNG2004170 | 0 | 0 | 0 | 0 | 0 | 6.209 | 0 |
| HLUNG2005656 | 0 | 0 | 0 | 16.214 | 0 | 0 | 0 |
| HLUNG2006812 | 0 | 0 | 0 | 2.099 | 0 | 0 | 0 |
| HLUNG2007163 | 0 | 0 | 0 | 0 | 0 | 27.149 | 0 |
| HLUNG2008139 | 0 | 0 | 0 | 8.134 | 0 | 0 | 0 |
| HLUNG2008153 | 0 | 0 | 0 | 0 | 0 | 20.576 | 0 |
| HLUNG2008348 | 0 | 0 | 0 | 9.019 | 0 | 12.803 | 0 |
| HLUNG2008637 | 0 | 11.856 | 0 | 0 | 0 | 0 | 0 |
| HLUNG2008885 | 0 | 0 | 0 | 7.662 | 0 | 0 | 0 |
| HLUNG2009225 | 0 | 0 | 0 | 4.282 | 0 | 3.039 | 0 |
| HLUNG2017262 | 0 | 0 | 0 | 4.502 | 0 | 0 | 0 |
| HSYRA1000062 | 0 | 0 | 16.516 | 0.608 | 0 | 0.432 | 0 |
| HSYRA1000119 | 0 | 0 | 3.546 | 1.305 | 1.661 | 0.463 | 0 |
| HSYRA1000137 | 0 | 0 | 0 | 0 | 0 | 3.755 | 0 |
| HSYRA1000152 | 0 | 0 | 0 | 0 | 0 | 4.807 | 0 |
| HSYRA1000178 | 0 | 0 | 0 | 1.780 | 0 | 5.053 | 0 |
| HSYRA2000137 | 0 | 0 | 0 | 0 | 8.190 | 0 | 0 |
| HSYRA2000159 | 0 | 0.846 | 1.860 | 2.653 | 1.743 | 6.803 | 0 |
| HSYRA2000190 | 0 | 16.670 | 0 | 1.686 | 0 | 0 | 0 |
| HSYRA2000224 | 0 | 0 | 0 | 0 | 0 | 3.624 | 0 |
| HSYRA2000232 | 0 | 0 | 0 | 3.149 | 2.672 | 3.725 | 0 |
| HSYRA2000248 | 0 | 0 | 5.241 | 0.965 | 0 | 2.054 | 0 |
| HSYRA2000255 | 0 | 0 | 0 | 0 | 0 | 0.452 | 0 |
| HSYRA2000629 | 0 | 0 | 0 | 2.369 | 0 | 0 | 0 |
| HSYRA2000640 | 0 | 0 | 0 | 0 | 0 | 6.009 | 0 |
| HSYRA2000706 | 0 | 0 | 0 | 8.673 | 0 | 0 | 0 |
| HSYRA2000743 | 0 | 0 | 5.968 | 1.648 | 0 | 1.560 | 0 |
| HSYRA2000787 | 0 | 0 | 0 | 2.208 | 0 | 0 | 0 |
| HSYRA2000828 | 0 | 9.999 | 0 | 0 | 0 | 4.306 | 0 |
| HSYRA2000925 | 0 | 0 | 0 | 0.472 | 0.801 | 0 | 83.696 |
| HSYRA2001003 | 0 | 0 | 0 | 6.093 | 0 | 0 | 0 |
| HSYRA2001105 | 0 | 0 | 0 | 5.867 | 0 | 2.776 | 0 |
| HSYRA2001138 | 0 | 0 | 0 | 1.267 | 0 | 3.082 | 0 |
| HSYRA2001225 | 0 | 0 | 0 | 0.525 | 0 | 0 | 0 |
| HSYRA2001396 | 0 | 0 | 0 | 1.078 | 0 | 1.531 | 0 |
| HSYRA2001476 | 0 | 0 | 0 | 0 | 0 | 1.946 | 0 |
| HSYRA2001567 | 0 | 0 | 0 | 1.591 | 0 | 0 | 0 |
| HSYRA2001574 | 0 | 5.281 | 0 | 0 | 0 | 0.758 | 0 |
| HSYRA2001595 | 0 | 0 | 0 | 0.809 | 2.058 | 1.722 | 0 |
| HSYRA2001615 | 0 | 0 | 0 | 7.360 | 0 | 8.359 | 0 |
| HSYRA2001621 | 0 | 0 | 0 | 0 | 3.315 | 1.294 | 0 |
| HSYRA2001983 | 0 | 0 | 0 | 6.851 | 0 | 0 | 0 |
| IMR321000158 | 0 | 0 | 0 | 0 | 0 | 0.767 | 0 |
| IMR321000193 | 0 | 0 | 0 | 0 | 0 | 4.422 | 0 |
| IMR321000210 | 0 | 0 | 0 | 2.158 | 0 | 0 | 0 |
| IMR321000219 | 0 | 0 | 0 | 1.594 | 0 | 3.959 | 0 |
| IMR321000242 | 0 | 0 | 0 | 3.991 | 0 | 0 | 0 |
| IMR321000266 | 0 | 0 | 0 | 2.009 | 0 | 2.851 | 0 |
| IMR322000072 | 25.151 | 0 | 0 | 0 | 0 | 0 | 0 |
| IMR322000121 | 0 | 0 | 0 | 0.791 | 0 | 4.493 | 0 |
| IMR322000127 | 0 | 10.873 | 0 | 4.398 | 5.597 | 10.926 | 0 |
| IMR322000223 | 0 | 0 | 0 | 0.966 | 2.460 | 0 | 0 |
| IMR322000243 | 0 | 0 | 0 | 1.218 | 0 | 1.729 | 0 |
| IMR322000316 | 0 | 0 | 0 | 3.534 | 4.498 | 6.272 | 0 |
| IMR322000518 | 0 | 0 | 0 | 6.540 | 11.097 | 0 | 0 |
| IMR322000555 | 0 | 0 | 0 | 4.234 | 0 | 0 | 0 |
| IMR322000730 | 4.296 | 0.962 | 0 | 0 | 1.486 | 0 | 0 |
| IMR322000742 | 0 | 0 | 0 | 2.351 | 5.983 | 1.668 | 0 |
| IMR322000775 | 0 | 0 | 0 | 2.943 | 0 | 0 | 0 |
| IMR322000819 | 0 | 0 | 0 | 0 | 0 | 4.295 | 0 |
| IMR322000859 | 0 | 0 | 0 | 28.688 | 0 | 0 | 0 |
| IMR322000863 | 0 | 0 | 0 | 4.788 | 0 | 0 | 0 |
| IMR322000918 | 0 | 0 | 0 | 4.161 | 0 | 2.953 | 0 |
| IMR322000973 | 25.275 | 0 | 6.218 | 0 | 0 | 0 | 0 |
| IMR322001049 | 0 | 0 | 0 | 0 | 20.931 | 0 | 0 |
| IMR322001218 | 0 | 0 | 0 | 0.795 | 0 | 0 | 0 |
| IMR322001317 | 0 | 0 | 0 | 2.802 | 5.707 | 0 | 0 |
| IMR322001318 | 0 | 0 | 0 | 0 | 0 | 22.213 | 0 |
| IMR322001322 | 0 | 0 | 0 | 0 | 0 | 22.213 | 0 |
| IMR322001332 | 0 | 0 | 0 | 3.779 | 0 | 0 | 0 |
| IMR322001380 | 0 | 0 | 0 | 2.814 | 7.163 | 11.984 | 0 |
| IMR322001435 | 0 | 0 | 0 | 1.334 | 0 | 0.947 | 0 |
| IMR322001438 | 0 | 0 | 0 | 5.898 | 0 | 0 | 0 |
| IMR322001534 | 0 | 0 | 0 | 5.865 | 0 | 0 | 0 |
| IMR322001541 | 0 | 0 | 0 | 2.401 | 0 | 0 | 0 |
| IMR322001584 | 0 | 0 | 0 | 1.772 | 0 | 2.516 | 0 |
| IMR322001600 | 0 | 0 | 0 | 0 | 0 | 0.487 | 0 |
| IMR322001670 | 0 | 0 | 0 | 9.714 | 0 | 18.387 | 0 |
| IMR322009710 | 0 | 0 | 0 | 16.746 | 0 | 0 | 0 |
| IMR322019070 | 0 | 0 | 0 | 0 | 19.335 | 0 | 0 |
| KIDNE1000104 | 0 | 0 | 0 | 11.771 | 0 | 0 | 0 |
| KIDNE1000145 | 0 | 19.410 | 0 | 0 | 0 | 0 | 0 |
| KIDNE2000085 | 0 | 0 | 7.930 | 0.730 | 0 | 0 | 0 |
| KIDNE2000252 | 0 | 0 | 6.591 | 1.213 | 0 | 3.444 | 0 |
| KIDNE2000266 | 0 | 0 | 0 | 5.977 | 0 | 4.242 | 0 |
| KIDNE2000349 | 0 | 0 | 14.632 | 0 | 0 | 0 | 0 |
| KIDNE2000383 | 0 | 0 | 0 | 0 | 0 | 4.962 | 0 |
| KIDNE2000574 | 0 | 5.254 | 0 | 1.063 | 2.705 | 1.508 | 0 |
| KIDNE2000665 | 0 | 0 | 0 | 1.374 | 0 | 1.951 | 0 |
| KIDNE2000909 | 0 | 0 | 0 | 1.457 | 0 | 4.137 | 0 |
| KIDNE2000947 | 0 | 0 | 19.491 | 0 | 9.132 | 0 | 0 |
| KIDNE2001162 | 0 | 0 | 10.798 | 0.994 | 0 | 0.941 | 0 |
| KIDNE2001467 | 0 | 0 | 0 | 0.740 | 0 | 2.364 | 0 |
| KIDNE2002191 | 0 | 0 | 0 | 15.177 | 0 | 0 | 0 |
| KIDNE2002265 | 0 | 0 | 0 | 0 | 0 | 1.833 | 0 |
| KIDNE2002725 | 0 | 0 | 0 | 7.303 | 0 | 0 | 0 |
| KIDNE2003233 | 0 | 0 | 0 | 16.915 | 0 | 0 | 0 |
| KIDNE2003305 | 0 | 17.723 | 0 | 0.597 | 0 | 0 | 0 |
| KIDNE2003764 | 0 | 0 | 0 | 22.937 | 0 | 0 | 0 |
| KIDNE2003941 | 15.810 | 0 | 0 | 0 | 0 | 1.016 | 0 |
| KIDNE2004054 | 0 | 0 | 0 | 9.265 | 0 | 0 | 0 |
| KIDNE2004115 | 0 | 0 | 0 | 16.915 | 0 | 0 | 0 |
| KIDNE2004475 | 0 | 0 | 0 | 1.827 | 0 | 0 | 0 |
| KIDNE2005676 | 0 | 0 | 0 | 5.991 | 0 | 0 | 0 |
| KIDNE2006248 | 0 | 2.182 | 0 | 0 | 0 | 0.940 | 0 |
| KIDNE2006465 | 0 | 0 | 0 | 10.981 | 0 | 3.118 | 0 |
| KIDNE2006580 | 0 | 0 | 0 | 14.933 | 0 | 7.066 | 0 |
| KIDNE2006760 | 0 | 0 | 0 | 0 | 24.375 | 0 | 0 |
| KIDNE2006775 | 0 | 0 | 70.644 | 1.300 | 0 | 0 | 0 |
| KIDNE2006880 | 0 | 0 | 0 | 0 | 0 | 3.235 | 0 |
| KIDNE2007352 | 0 | 2.918 | 0 | 2.950 | 0 | 0.838 | 0 |
| KIDNE2007954 | 0 | 0 | 0 | 7.481 | 0 | 0 | 0 |
| KIDNE2008048 | 0 | 0 | 0 | 1.904 | 0 | 1.081 | 0 |
| KIDNE2008069 | 0 | 0 | 0 | 3.314 | 12.653 | 9.409 | 0 |
| KIDNE2008362 | 0 | 0 | 0 | 0 | 16.499 | 0 | 0 |
| KIDNE2008403 | 0 | 0 | 0 | 9.653 | 0 | 19.184 | 0 |
| KIDNE2009367 | 0 | 0 | 0 | 7.489 | 2.723 | 6.075 | 0 |
| KIDNE2011752 | 0 | 0 | 0 | 16.915 | 0 | 0 | 0 |
| KIDNE2012009 | 0 | 66.814 | 0 | 0 | 0 | 0 | 0 |
| KIDNE2012291 | 0 | 0 | 0 | 16.915 | 0 | 0 | 0 |
| KIDNE2012440 | 0 | 0 | 0 | 2.513 | 0 | 0 | 0 |
| KIDNE2013845 | 0 | 0 | 0 | 0.793 | 0 | 0 | 0 |
| KIDNE2014290 | 0 | 0 | 0 | 0 | 0 | 0.706 | 0 |
| KIDNE2014978 | 0 | 0 | 0 | 0 | 0 | 22.421 | 0 |
| KIDNE2015710 | 0 | 13.138 | 0 | 0 | 0 | 0 | 0 |
| KIDNE2016101 | 0 | 0 | 0 | 16.915 | 0 | 0 | 0 |
| KIDNE2016371 | 0 | 0 | 0 | 0 | 0 | 9.078 | 0 |
| KIDNE2017040 | 0 | 0 | 0 | 2.387 | 0 | 0 | 0 |
| KIDNE2017153 | 0 | 0 | 0 | 16.915 | 0 | 0 | 0 |
| KIDNE2018071 | 0 | 0 | 0 | 0 | 50.895 | 0 | 0 |
| KIDNE2018217 | 0 | 0 | 0 | 4.307 | 0 | 0 | 0 |
| KIDNE2018493 | 0 | 0 | 0 | 0 | 0 | 3.590 | 0 |
| KIDNE2018891 | 0 | 0 | 0 | 0 | 22.035 | 0 | 0 |
| LIVER1000079 | 0 | 0 | 0 | 2.487 | 0 | 5.297 | 0 |
| LIVER1000104 | 0 | 0 | 0 | 0 | 0 | 0.208 | 0 |
| LIVER1000132 | 0 | 3.512 | 0 | 0 | 0 | 0 | 0 |
| LIVER1000230 | 0 | 0 | 0 | 8.232 | 0 | 0 | 0 |
| LIVER2000033 | 0 | 0 | 0 | 0 | 0 | 1.394 | 0 |
| LIVER2000037 | 0 | 0 | 0 | 0 | 0.229 | 0 | 0 |
| LIVER2001265 | 0 | 0 | 0 | 0 | 0 | 10.413 | 0 |
| LIVER2002842 | 0 | 0 | 0 | 0 | 0 | 1.924 | 0 |
| LIVER2003008 | 0 | 0 | 0 | 0 | 0 | 10.413 | 0 |
| LIVER2006006 | 0 | 0 | 5.484 | 0 | 0 | 0 | 0 |
| LIVER2007783 | 0 | 0 | 0 | 1.456 | 0 | 2.067 | 0 |
| LIVER2008465 | 0 | 0 | 0 | 4.697 | 0 | 0 | 0 |
| MAMGL1000032 | 0 | 2.262 | 0 | 0.114 | 0.582 | 0.487 | 0 |
| MAMGL1000035 | 0 | 0 | 0 | 0.143 | 0 | 0.203 | 0 |
| MAMGL1000083 | 0 | 0 | 0 | 0.206 | 0 | 0 | 0 |
| MAMGL1000100 | 0 | 0 | 0 | 0.210 | 0 | 0 | 0 |
| MAMGL1000173 | 0 | 1.860 | 0 | 0 | 0 | 0 | 0 |
| MESAN1000050 | 0 | 0 | 0 | 0 | 0 | 3.235 | 0 |
| MESAN1000080 | 0 | 0 | 0 | 0 | 0 | 1.439 | 0 |
| MESAN1000101 | 0 | 0 | 0 | 4.097 | 0 | 0 | 0 |
| MESAN1000126 | 0 | 0 | 0 | 7.605 | 0 | 0 | 0 |
| MESAN2000267 | 0 | 0 | 0 | 1.744 | 1.480 | 2.888 | 0 |
| MESAN2000501 | 0 | 0 | 0 | 0 | 0 | 3.023 | 0 |
| MESAN2000572 | 0 | 0 | 0 | 0 | 0 | 1.030 | 0 |
| MESAN2000608 | 0 | 0 | 6.064 | 1.116 | 0 | 0.792 | 0 |
| MESAN2001154 | 0 | 0 | 0 | 4.728 | 0 | 0 | 0 |
| MESAN2001450 | 0 | 0 | 0 | 6.141 | 0 | 0 | 0 |
| MESAN2002086 | 0 | 2.922 | 0 | 0 | 1.504 | 0 | 0 |
| MESAN2002122 | 0 | 0 | 0 | 6.296 | 0 | 0 | 0 |
| MESAN2002186 | 0 | 0 | 16.905 | 0 | 0 | 0 | 0 |
| MESAN2002519 | 0 | 0 | 0 | 0 | 0 | 4.673 | 0 |
| MESAN2002724 | 0 | 0 | 0 | 0 | 0 | 1.858 | 0 |
| MESAN2003622 | 18.837 | 4.218 | 0 | 1.280 | 0 | 0 | 0 |
| MESAN2003709 | 0 | 0 | 0 | 0 | 29.970 | 0 | 0 |
| MESAN2005689 | 0 | 0 | 0 | 0 | 11.210 | 3.126 | 0 |
| MESAN2005766 | 0 | 0 | 0 | 2.100 | 0 | 4.968 | 0 |
| MESAN2005957 | 0 | 0 | 0 | 7.009 | 0 | 0 | 0 |
| MESAN2005958 | 0 | 0 | 0 | 3.823 | 9.731 | 0 | 0 |
| MESAN2006401 | 0 | 0 | 0 | 0 | 0 | 10.937 | 0 |
| MESAN2006563 | 0 | 0 | 0 | 1.470 | 0 | 0 | 0 |
| MESAN2008334 | 0 | 0 | 0 | 11.670 | 0 | 0 | 0 |
| MESAN2008729 | 0 | 0 | 0 | 16.066 | 0 | 0 | 0 |
| MESAN2008936 | 0 | 0 | 0 | 5.647 | 0 | 12.025 | 0 |
| MESAN2010664 | 0 | 0 | 0 | 6.160 | 0 | 0 | 0 |
| MESAN2011984 | 0 | 0 | 0 | 0 | 0 | 1.719 | 0 |
| MESAN2012054 | 0 | 0 | 0 | 5.432 | 0 | 0 | 0 |
| MESAN2013284 | 0 | 0 | 0 | 2.615 | 0 | 6.187 | 0 |
| MESAN2015365 | 0 | 0 | 0 | 16.066 | 0 | 0 | 0 |
| MESAN2015515 | 0 | 0 | 20.486 | 0 | 0 | 2.677 | 0 |
| MESAN2017133 | 0 | 0 | 0 | 0 | 31.703 | 0 | 0 |
| MESAN2017373 | 0 | 0 | 0 | 0 | 0 | 3.529 | 0 |
| MESAN2018699 | 0 | 0 | 0 | 0 | 0 | 15.512 | 0 |
| MESTC1000042 | 0 | 0 | 0 | 0.797 | 0 | 0 | 0 |
| NB9N41000011 | 0 | 0 | 0 | 4.532 | 0 | 0 | 0 |
| NB9N41000121 | 0 | 0 | 5.791 | 0 | 0 | 0 | 0 |
| NB9N41000135 | 0 | 0 | 0 | 1.574 | 0 | 0 | 0 |
| NB9N41000340 | 0 | 0 | 11.184 | 2.059 | 0 | 0 | 0 |
| NB9N42000196 | 31.658 | 0 | 0 | 0 | 0 | 0 | 0 |
| NESOP2001752 | 0 | 0 | 0 | 3.228 | 0 | 0 | 0 |
| NHNPC1000034 | 0 | 0 | 0 | 0 | 0 | 3.882 | 0 |
| NHNPC2000062 | 0 | 0 | 0 | 0 | 0 | 2.112 | 0 |
| NHNPC2000187 | 0 | 0 | 0 | 0.321 | 0 | 1.824 | 0 |
| NHNPC2000206 | 0 | 0 | 0 | 3.412 | 0 | 0.692 | 0 |
| NHNPC2001931 | 0 | 3.350 | 0 | 0 | 0 | 0.481 | 0 |
| NOVAR2000038 | 0 | 0 | 0 | 3.968 | 15.711 | 0.626 | 0 |
| NOVAR2000783 | 0 | 0 | 0 | 2.684 | 0 | 0 | 0 |
| NT2NE1000014 | 0 | 0 | 0 | 6.170 | 0 | 0 | 0 |
| NT2NE1000018 | 0 | 0 | 19.177 | 1.765 | 0 | 10.022 | 0 |
| NT2NE1000163 | 0 | 0 | 0 | 4.025 | 1.464 | 1.224 | 0 |
| NT2NE2000056 | 0 | 2.757 | 3.029 | 1.115 | 0 | 5.541 | 0 |
| NT2NE2000174 | 0 | 0 | 5.885 | 0.271 | 0 | 0.384 | 0 |
| NT2NE2000195 | 0 | 0 | 0 | 2.182 | 5.554 | 1.549 | 0 |
| NT2NE2000259 | 0 | 0 | 0 | 1.996 | 0 | 2.834 | 0 |
| NT2NE2000260 | 0 | 4.239 | 0 | 0 | 2.182 | 0.852 | 0 |
| NT2NE2000327 | 0 | 0 | 0 | 5.357 | 0 | 0 | 0 |
| NT2NE2000383 | 0 | 0 | 0 | 3.236 | 5.491 | 1.531 | 0 |
| NT2NE2000384 | 0 | 0 | 0 | 1.634 | 0 | 2.319 | 0 |
| NT2NE2000392 | 0 | 0 | 0 | 3.168 | 0 | 3.598 | 0 |
| NT2NE2000517 | 0 | 0 | 0 | 0 | 0 | 14.309 | 0 |
| NT2NE2000575 | 0 | 1.687 | 0 | 1.706 | 0.869 | 1.453 | 0 |
| NT2NE2000707 | 0 | 4.698 | 0 | 1.188 | 0 | 2.697 | 0 |
| NT2NE2000763 | 0 | 0 | 0 | 0 | 0 | 2.418 | 0 |
| NT2NE2000864 | 0 | 0 | 0 | 16.377 | 0 | 0 | 0 |
| NT2NE2000909 | 0 | 0 | 0 | 0 | 11.828 | 13.193 | 0 |
| NT2NE2000913 | 0 | 0 | 0 | 5.235 | 0 | 0 | 0 |
| NT2NE2000963 | 0 | 3.220 | 0 | 0.814 | 3.316 | 0 | 0 |
| NT2NE2001000 | 26.976 | 0 | 0 | 1.222 | 0 | 0.867 | 0 |
| NT2NE2001040 | 0 | 0 | 0 | 11.003 | 0 | 15.621 | 0 |
| NT2NE2001048 | 0 | 10.506 | 0 | 2.125 | 10.817 | 1.508 | 0 |
| NT2NE2001156 | 0 | 0 | 0 | 0 | 20.308 | 0 | 0 |
| NT2NE2001176 | 0 | 0 | 0 | 1.621 | 4.127 | 6.905 | 0 |
| NT2NE2001324 | 0 | 0 | 0 | 16.377 | 0 | 0 | 0 |
| NT2NE2001337 | 0 | 0 | 0 | 6.451 | 0 | 0 | 0 |
| NT2NE2001364 | 0 | 0 | 0 | 0 | 0 | 10.772 | 0 |
| NT2NE2001397 | 0 | 0 | 0 | 0 | 0 | 13.252 | 0 |
| NT2NE2001403 | 0 | 0 | 0 | 3.682 | 0 | 0 | 0 |
| NT2NE2001435 | 0 | 0 | 0 | 6.037 | 3.841 | 4.285 | 0 |
| NT2NE2001442 | 0 | 35.075 | 0 | 0 | 0 | 0 | 0 |
| NT2NE2001545 | 0 | 0 | 0 | 0 | 0 | 3.611 | 0 |
| NT2NE2001655 | 0 | 0 | 0 | 0 | 5.059 | 1.411 | 0 |
| NT2NE2001697 | 0 | 0 | 0 | 0 | 0 | 5.501 | 0 |
| NT2NE2001793 | 0 | 0 | 0 | 2.945 | 0 | 4.180 | 0 |
| NT2NE2003185 | 0 | 0 | 0 | 2.690 | 13.697 | 0 | 0 |
| NT2NE2003485 | 0 | 0 | 0 | 1.652 | 0 | 0 | 0 |
| NT2NE2003569 | 0 | 0 | 0 | 1.232 | 0 | 1.749 | 0 |
| NT2NE2003921 | 0 | 0 | 0 | 4.330 | 0 | 0 | 0 |
| NT2NE2004519 | 0 | 0 | 0 | 0 | 0 | 1.806 | 0 |
| NT2NE2005317 | 0 | 0 | 0 | 9.025 | 0 | 0 | 0 |
| NT2NE2005419 | 0 | 0 | 0 | 0 | 0 | 11.667 | 0 |
| NT2NE2005688 | 0 | 0 | 0 | 0.530 | 0 | 0 | 0 |
| NT2NE2005876 | 0 | 0 | 0 | 5.235 | 0 | 2.973 | 0 |
| NT2NE2005890 | 0 | 0 | 0 | 0 | 0 | 35.735 | 0 |
| NT2NE2006458 | 0 | 0 | 0 | 2.006 | 0 | 1.424 | 0 |
| NT2NE2006659 | 0 | 0 | 0 | 12.843 | 0 | 0 | 0 |
| NT2NE2006909 | 0 | 0 | 0 | 0 | 0 | 0.287 | 0 |
| NT2NE2008803 | 0 | 0 | 0 | 16.377 | 0 | 0 | 0 |
| NT2NE2009523 | 0 | 0 | 0 | 0 | 0 | 15.659 | 0 |
| NT2NE2010056 | 0 | 0 | 0 | 17.062 | 0 | 16.148 | 0 |
| NT2NE2010842 | 0 | 0 | 0 | 0.669 | 0 | 3.800 | 0 |
| NT2NE2011119 | 0 | 0 | 0 | 0 | 0 | 4.665 | 0 |
| NT2NE2012199 | 0 | 0 | 0 | 0 | 0 | 12.205 | 0 |
| NT2NE2012505 | 0 | 0 | 0 | 13.014 | 0 | 0 | 0 |
| NT2NE2012928 | 0 | 0 | 0 | 6.179 | 0 | 0 | 0 |
| NT2NE2014104 | 0 | 23.562 | 0 | 0 | 0 | 1.691 | 0 |
| NT2NE2015362 | 0 | 0 | 0 | 13.014 | 0 | 0 | 0 |
| NT2NE2015974 | 0 | 0 | 0 | 21.687 | 0 | 7.697 | 0 |
| NT2NE2017792 | 0 | 0 | 0 | 13.288 | 0 | 18.864 | 0 |
| NT2NE2018180 | 0 | 0 | 0 | 2.669 | 0 | 0 | 0 |
| NT2NE2019091 | 0 | 0 | 0 | 0 | 0 | 1.907 | 0 |
| NT2RI1000027 | 0 | 0 | 0 | 1.435 | 0 | 1.019 | 0 |
| NT2RI1000127 | 0 | 0 | 0 | 1.288 | 0 | 0 | 0 |
| NT2RI2000064 | 0 | 3.778 | 8.302 | 0.382 | 0 | 0.542 | 0 |
| NT2RI2000107 | 0 | 0 | 0 | 0 | 0 | 2.334 | 0 |
| NT2RI2000116 | 0 | 0 | 0 | 1.238 | 0 | 2.344 | 0 |
| NT2RI2000133 | 0 | 0 | 0 | 1.819 | 0 | 0 | 0 |
| NT2RI2000167 | 0 | 0 | 0 | 0 | 0 | 11.641 | 0 |
| NT2RI2000255 | 0 | 0 | 9.231 | 0 | 0 | 2.412 | 0 |
| NT2RI2000263 | 0 | 0 | 0 | 1.214 | 0 | 0 | 0 |
| NT2RI2000282 | 9.921 | 0 | 9.764 | 0.449 | 0 | 0 | 0 |
| NT2RI2000284 | 0 | 0 | 0 | 0.324 | 1.647 | 0.919 | 0 |
| NT2RI2000294 | 0 | 0 | 0 | 1.343 | 0 | 3.814 | 0 |
| NT2RI2000344 | 0 | 0 | 0 | 1.149 | 0 | 0.816 | 0 |
| NT2RI2000412 | 0 | 0 | 0 | 0 | 0 | 23.298 | 0 |
| NT2RI2000578 | 0 | 0 | 0 | 0 | 0 | 0.674 | 0 |
| NT2RI2000671 | 0 | 0 | 0 | 0 | 0 | 1.060 | 0 |
| NT2RI2000685 | 0 | 0 | 0 | 2.813 | 0 | 2.995 | 0 |
| NT2RI2000727 | 0 | 1.816 | 3.990 | 0.184 | 0 | 2.607 | 0 |
| NT2RI2000829 | 0 | 0 | 0 | 8.133 | 13.801 | 0 | 0 |
| NT2RI2001054 | 0 | 0 | 4.588 | 0.845 | 0 | 1.199 | 0 |
| NT2RI2001091 | 0 | 24.677 | 0 | 2.495 | 0 | 0 | 0 |
| NT2RI2001230 | 0 | 5.414 | 0 | 0 | 0 | 0.777 | 0 |
| NT2RI2001342 | 0 | 11.079 | 0 | 0 | 0 | 0 | 0 |
| NT2RI2001385 | 0 | 0 | 0 | 0 | 14.236 | 0 | 0 |
| NT2RI2001422 | 0 | 0 | 0 | 5.277 | 0 | 0 | 0 |
| NT2RI2001450 | 0 | 0 | 0 | 2.515 | 0 | 16.068 | 0 |
| NT2RI2001540 | 0 | 1.998 | 0.439 | 1.333 | 0.617 | 0.229 | 0 |
| NT2RI2001621 | 0 | 0 | 0 | 2.173 | 0 | 0 | 0 |
| NT2RI2001731 | 0 | 0 | 0 | 1.344 | 0 | 0 | 0 |
| NT2RI2001769 | 0 | 0 | 17.143 | 0 | 0 | 0 | 0 |
| NT2RI2001859 | 0 | 0 | 0 | 0.689 | 0 | 1.956 | 0 |
| NT2RI2001866 | 0 | 0 | 0 | 10.300 | 0 | 0 | 0 |
| NT2RI2002091 | 1.261 | 0.659 | 2.586 | 0.048 | 0.097 | 0.176 | 0 |
| NT2RI2002152 | 0 | 0 | 0 | 0.893 | 0 | 2.534 | 0 |
| NT2RI2002243 | 0 | 0 | 0 | 0 | 0 | 3.314 | 0 |
| NT2RI2002266 | 0 | 0 | 0 | 0 | 0 | 2.525 | 0 |
| NT2RI2002316 | 0 | 0 | 0 | 0.569 | 0 | 0 | 0 |
| NT2RI2002391 | 0 | 0 | 0 | 0 | 0 | 4.086 | 0 |
| NT2RI2002540 | 0 | 0 | 0 | 1.227 | 0 | 6.967 | 0 |
| NT2RI2002554 | 0 | 12.727 | 0 | 0 | 0 | 1.827 | 0 |
| NT2RI2002585 | 0 | 0 | 0 | 9.228 | 0 | 0 | 0 |
| NT2RI2002654 | 0 | 0 | 0 | 1.668 | 0 | 1.184 | 0 |
| NT2RI2002699 | 0 | 0 | 0 | 0 | 0 | 11.520 | 0 |
| NT2RI2002802 | 0 | 0 | 0 | 0 | 0 | 28.966 | 0 |
| NT2RI2002819 | 0 | 0 | 0 | 0 | 0 | 3.751 | 0 |
| NT2RI2002847 | 0 | 0 | 0 | 0 | 0 | 1.265 | 0 |
| NT2RI2002958 | 0 | 0 | 0 | 0 | 0 | 2.910 | 0 |
| NT2RI2003011 | 0 | 0 | 0 | 2.451 | 0 | 3.479 | 0 |
| NT2RI2003051 | 0 | 0 | 0 | 3.633 | 0 | 2.063 | 0 |
| NT2RI2003205 | 0 | 0 | 0 | 0 | 0 | 3.244 | 0 |
| NT2RI2003222 | 0 | 0 | 0 | 0 | 0 | 25.614 | 0 |
| NT2RI2003301 | 0 | 0 | 0 | 0 | 0 | 5.719 | 0 |
| NT2RI2003304 | 0 | 0 | 2.024 | 0 | 0 | 0.529 | 0 |
| NT2RI2003317 | 0 | 11.064 | 0 | 0 | 0 | 4.765 | 0 |
| NT2RI2003338 | 0 | 0 | 0 | 0 | 0 | 3.061 | 0 |
| NT2RI2003344 | 0 | 0 | 0 | 9.675 | 0 | 1.962 | 0 |
| NT2RI2003361 | 0 | 0 | 0 | 3.505 | 0 | 0 | 0 |
| NT2RI2003407 | 0 | 0 | 0 | 0.859 | 0 | 1.220 | 0 |
| NT2RI2003420 | 0 | 0 | 0 | 2.186 | 0 | 1.552 | 0 |
| NT2RI2003481 | 0 | 0 | 0 | 8.223 | 13.955 | 5.837 | 0 |
| NT2RI2003556 | 0 | 0 | 0 | 0.270 | 1.375 | 0.767 | 0 |
| NT2RI2003655 | 0 | 0 | 0 | 0 | 0 | 19.683 | 0 |
| NT2RI2003678 | 0 | 0 | 0 | 0 | 0 | 3.837 | 0 |
| NT2RI2003695 | 0 | 0 | 12.145 | 0 | 0 | 5.554 | 0 |
| NT2RI2003738 | 0 | 0 | 0 | 0.124 | 0.632 | 0.529 | 0 |
| NT2RI2003921 | 0 | 0 | 0 | 0 | 0 | 1.911 | 0 |
| NT2RI2004059 | 0 | 0 | 0 | 0 | 24.622 | 0 | 0 |
| NT2RI2004284 | 0 | 0 | 0 | 1.141 | 0 | 3.239 | 0 |
| NT2RI2004290 | 0 | 0 | 0 | 0 | 0 | 21.496 | 0 |
| NT2RI2004304 | 0 | 0 | 0 | 5.911 | 0 | 4.196 | 0 |
| NT2RI2004398 | 0 | 34.966 | 0 | 3.536 | 0 | 5.020 | 0 |
| NT2RI2004410 | 0 | 0 | 0 | 5.311 | 18.024 | 5.026 | 0 |
| NT2RI2004535 | 0 | 0 | 0 | 2.717 | 0 | 3.857 | 0 |
| NT2RI2004608 | 0 | 25.968 | 0 | 0 | 0 | 0 | 0 |
| NT2RI2005115 | 0 | 0 | 0 | 4.337 | 0 | 6.157 | 0 |
| NT2RI2005116 | 0 | 8.972 | 0 | 1.815 | 0 | 1.288 | 0 |
| NT2RI2005150 | 0 | 0 | 0 | 0.489 | 0 | 4.860 | 0 |
| NT2RI2005166 | 0 | 0 | 0 | 18.511 | 0 | 0 | 0 |
| NT2RI2005239 | 0 | 0 | 0 | 0 | 0 | 7.947 | 0 |
| NT2RI2005246 | 0 | 0 | 71.596 | 0 | 0 | 0 | 0 |
| NT2RI2005335 | 12.134 | 0 | 0 | 5.495 | 0 | 2.730 | 0 |
| NT2RI2005353 | 0 | 0 | 0 | 0.818 | 0 | 2.321 | 0 |
| NT2RI2005405 | 0 | 0 | 0 | 0.199 | 0 | 0 | 0 |
| NT2RI2005473 | 0 | 22.924 | 0 | 0 | 0 | 0 | 0 |
| NT2RI2005564 | 0 | 0 | 0 | 6.323 | 0 | 0 | 0 |
| NT2RI2005713 | 0 | 0 | 0 | 0 | 5.344 | 2.980 | 0 |
| NT2RI2005723 | 0 | 0 | 0 | 1.781 | 0 | 1.011 | 0 |
| NT2RI2005811 | 0 | 10.753 | 0 | 0 | 0 | 3.087 | 0 |
| NT2RI2005816 | 0 | 0 | 0 | 0 | 0 | 6.440 | 0 |
| NT2RI2005851 | 0 | 0 | 0 | 5.290 | 0 | 11.265 | 0 |
| NT2RI2006070 | 0 | 0 | 0 | 0 | 0 | 2.071 | 0 |
| NT2RI2006071 | 0 | 0 | 0 | 64.115 | 0 | 0 | 0 |
| NT2RI2006072 | 0 | 0 | 0 | 0 | 0 | 0.771 | 0 |
| NT2RI2006271 | 0 | 0 | 0 | 1.982 | 0 | 0 | 0 |
| NT2RI2006412 | 0 | 0 | 0 | 4.654 | 0 | 18.171 | 0 |
| NT2RI2006640 | 0 | 0 | 0 | 0 | 0 | 12.665 | 0 |
| NT2RI2006647 | 0 | 0 | 0 | 3.255 | 0 | 0 | 0 |
| NT2RI2006682 | 0 | 0 | 0 | 0 | 0 | 2.479 | 0 |
| NT2RI2006825 | 0 | 0 | 0 | 0 | 0 | 2.762 | 0 |
| NT2RI2006856 | 0 | 0 | 3.612 | 0.665 | 1.692 | 0.472 | 0 |
| NT2RI2006943 | 0 | 0 | 0 | 1.726 | 0 | 4.902 | 0 |
| NT2RI2007054 | 0 | 0 | 0 | 2.764 | 0 | 0 | 0 |
| NT2RI2007084 | 0 | 0 | 0 | 0.772 | 0 | 1.096 | 0 |
| NT2RI2007096 | 0 | 0 | 0 | 0 | 18.731 | 0 | 0 |
| NT2RI2007148 | 0 | 0 | 0 | 0.670 | 0 | 0.952 | 0 |
| NT2RI2007377 | 0 | 0 | 0 | 2.494 | 0 | 11.801 | 0 |
| NT2RI2007384 | 0 | 0 | 0 | 0 | 0 | 1.745 | 0 |
| NT2RI2007445 | 0 | 0 | 0 | 1.273 | 0 | 0 | 0 |
| NT2RI2007468 | 0 | 0 | 0 | 2.966 | 0 | 0 | 0 |
| NT2RI2007498 | 0 | 0 | 3.145 | 0 | 0 | 0 | 0 |
| NT2RI2007572 | 0 | 0 | 0 | 1.061 | 0 | 0 | 0 |
| NT2RI2007589 | 0 | 0 | 0 | 1.516 | 0 | 1.076 | 0 |
| NT2RI2007593 | 0 | 0 | 0 | 2.338 | 11.905 | 0 | 0 |
| NT2RI2007629 | 0 | 0 | 0 | 1.431 | 0 | 4.062 | 0 |
| NT2RI2007723 | 0 | 0 | 27.282 | 0 | 0 | 3.565 | 0 |
| NT2RI2007879 | 49.750 | 0 | 12.240 | 0 | 0 | 0 | 0 |
| NT2RI2007884 | 0 | 0 | 0 | 1.802 | 0 | 0 | 0 |
| NT2RI2007987 | 0 | 0 | 0 | 1.278 | 0 | 3.628 | 0 |
| NT2RI2008045 | 0 | 0 | 0 | 1.102 | 0 | 0 | 0 |
| NT2RI2008141 | 0 | 0 | 0 | 0 | 0 | 2.155 | 0 |
| NT2RI2008171 | 0 | 0 | 0 | 3.152 | 0 | 4.474 | 0 |
| NT2RI2008188 | 0 | 0 | 0 | 0 | 0 | 28.966 | 0 |
| NT2RI2008336 | 0 | 2.812 | 0 | 0.853 | 0 | 0.404 | 0 |
| NT2RI2008396 | 0 | 0 | 0 | 0.313 | 0 | 1.777 | 0 |
| NT2RI2008481 | 0 | 0 | 0 | 0 | 0 | 0.745 | 0 |
| NT2RI2008526 | 0 | 0 | 5.913 | 0 | 0 | 0 | 0 |
| NT2RI2008566 | 0 | 0 | 0 | 0 | 0 | 9.477 | 0 |
| NT2RI2008714 | 0 | 0 | 0 | 0 | 0 | 29.569 | 0 |
| NT2RI2008724 | 0 | 0 | 0 | 0 | 0 | 1.850 | 0 |
| NT2RI2008749 | 0 | 0 | 0 | 0.401 | 0 | 0.570 | 0 |
| NT2RI2008791 | 0 | 0 | 0 | 0.828 | 0 | 0 | 0 |
| NT2RI2008808 | 0 | 0 | 0 | 3.218 | 0 | 4.568 | 0 |
| NT2RI2008812 | 0 | 0 | 0 | 0 | 0 | 7.295 | 0 |
| NT2RI2008942 | 0 | 0 | 0 | 1.489 | 0 | 1.057 | 0 |
| NT2RI2009037 | 0 | 5.917 | 6.501 | 1.795 | 0 | 1.699 | 0 |
| NT2RI2009065 | 0 | 0 | 0 | 0.994 | 3.375 | 0.941 | 0 |
| NT2RI2009066 | 0 | 0 | 0 | 1.945 | 0 | 0 | 0 |
| NT2RI2009083 | 0 | 0 | 0 | 0 | 0 | 10.331 | 0 |
| NT2RI2009173 | 0 | 0 | 0 | 0 | 0 | 1.124 | 0 |
| NT2RI2009194 | 0 | 0 | 0 | 0 | 0 | 0.990 | 0 |
| NT2RI2009233 | 0 | 0 | 0 | 1.601 | 0 | 4.545 | 0 |
| NT2RI2009239 | 0 | 0 | 0 | 2.976 | 0 | 0 | 0 |
| NT2RI2009269 | 0 | 0 | 0 | 2.104 | 7.141 | 4.978 | 0 |
| NT2RI2009281 | 0 | 0 | 0 | 0.999 | 0 | 4.254 | 0 |
| NT2RI2009402 | 0 | 0 | 0 | 2.226 | 11.333 | 6.320 | 0 |
| NT2RI2009406 | 0 | 0 | 0 | 1.997 | 0 | 0 | 0 |
| NT2RI2009583 | 0 | 0 | 3.451 | 5.399 | 0.808 | 0.902 | 0 |
| NT2RI2010795 | 0 | 0 | 0 | 0 | 0 | 16.619 | 0 |
| NT2RI2012350 | 0 | 2.809 | 0 | 0 | 0 | 0 | 0 |
| NT2RI2012659 | 0 | 0 | 0 | 48.128 | 0 | 13.665 | 0 |
| NT2RI2012726 | 0 | 0 | 0 | 3.293 | 0 | 0 | 0 |
| NT2RI2015342 | 0 | 0 | 0 | 2.135 | 0 | 0 | 0 |
| NT2RI2015533 | 0 | 0 | 0 | 3.309 | 5.616 | 12.527 | 0 |
| NT2RI2019751 | 0 | 0 | 0 | 28.087 | 0 | 0 | 0 |
| NT2RI2025075 | 0 | 0 | 0 | 12.033 | 0 | 0 | 0 |
| NT2RI2027975 | 0 | 0 | 0 | 28.087 | 0 | 0 | 0 |
| NT2RI3001348 | 0 | 0 | 0 | 7.418 | 0 | 0 | 0 |
| NT2RI3001445 | 0 | 0 | 0 | 4.495 | 0 | 0 | 0 |
| NT2RI3001515 | 0 | 0 | 0 | 0 | 0 | 21.825 | 0 |
| NT2RI3003382 | 0 | 0 | 0 | 28.087 | 0 | 0 | 0 |
| NT2RI3004510 | 0 | 0 | 0 | 20.009 | 0 | 0 | 0 |
| NT2RI3005724 | 0 | 0 | 0 | 42.065 | 0 | 5.972 | 0 |
| NT2RI3006284 | 0 | 0 | 0 | 1.469 | 0 | 0 | 0 |
| NT2RI3006673 | 0 | 0 | 0 | 0 | 0 | 10.055 | 0 |
| NT2RI3007213 | 0 | 0 | 0 | 28.087 | 0 | 0 | 0 |
| NT2RI3007291 | 0 | 0 | 0 | 35.535 | 0 | 0 | 0 |
| NT2RI3007543 | 0 | 0 | 0 | 1.204 | 0 | 0.855 | 0 |
| NT2RI3008055 | 0 | 0 | 0 | 15.678 | 0 | 0 | 0 |
| NT2RP6000005 | 0 | 0 | 0 | 1.561 | 0 | 0 | 0 |
| NT2RP6000008 | 0 | 0 | 0 | 22.750 | 0 | 0 | 0 |
| NT2RP6000017 | 0 | 0 | 0 | 3.186 | 0 | 1.508 | 0 |
| NT2RP6000032 | 0 | 0 | 0 | 0 | 0 | 7.902 | 0 |
| NT2RP6000039 | 0 | 0 | 0 | 6.364 | 0 | 1.807 | 0 |
| NT2RP6000059 | 0 | 0 | 0 | 0 | 0 | 21.855 | 0 |
| NT2RP6000072 | 0 | 0 | 12.461 | 0 | 0 | 0 | 0 |
| NT2RP6000078 | 0 | 0 | 0 | 0.255 | 0 | 1.449 | 0 |
| NT2RP6000085 | 0 | 0 | 0 | 3.283 | 0 | 4.660 | 0 |
| NT2RP6000086 | 0 | 0 | 0 | 22.750 | 0 | 0 | 0 |
| NT2RP6000109 | 0 | 0 | 0 | 1.356 | 0 | 3.849 | 0 |
| NT2RP6000110 | 0 | 0 | 0 | 1.944 | 0 | 13.796 | 0 |
| NT2RP6000123 | 0 | 0 | 0 | 0.278 | 2.829 | 0.394 | 0 |
| NT2RP6000130 | 0 | 56.184 | 0 | 0 | 0 | 0 | 0 |
| NT2RP7000069 | 0 | 0 | 0 | 9.563 | 0 | 0 | 0 |
| NT2RP7000076 | 0 | 0 | 0 | 0 | 0 | 1.228 | 0 |
| NT2RP7000086 | 0 | 0 | 0 | 18.153 | 0 | 12.885 | 0 |
| NT2RP7000173 | 0 | 0 | 0 | 1.973 | 0 | 0.560 | 0 |
| NT2RP7000238 | 0 | 0 | 0 | 5.513 | 0 | 1.304 | 0 |
| NT2RP7000271 | 0 | 0 | 0 | 0 | 0 | 3.056 | 0 |
| NT2RP7000477 | 0 | 0 | 0 | 1.404 | 0 | 0 | 0 |
| NT2RP7000579 | 0 | 0 | 0 | 12.835 | 0 | 0 | 0 |
| NT2RP7000586 | 0 | 0 | 0 | 0 | 0 | 11.071 | 0 |
| NT2RP7000600 | 0 | 0 | 0 | 6.291 | 0 | 8.931 | 0 |
| NT2RP7000624 | 0 | 0 | 0 | 21.677 | 0 | 0 | 0 |
| NT2RP7000812 | 0 | 0 | 61.537 | 0 | 0 | 0 | 0 |
| NT2RP7000906 | 0 | 9.156 | 0 | 2.778 | 0 | 5.258 | 0 |
| NT2RP7001166 | 0 | 0 | 11.535 | 1.062 | 0 | 0.754 | 0 |
| NT2RP7001231 | 0 | 0 | 0 | 0 | 0 | 4.161 | 0 |
| NT2RP7001283 | 0 | 0 | 4.341 | 0.400 | 0 | 0 | 0 |
| NT2RP7001364 | 0 | 0 | 0 | 0 | 0 | 29.482 | 0 |
| NT2RP7001475 | 0 | 0 | 0 | 24.938 | 0 | 0 | 0 |
| NT2RP7001962 | 0 | 18.645 | 0 | 1.885 | 0 | 5.353 | 0 |
| NT2RP7002379 | 0 | 0 | 0 | 2.144 | 0 | 3.043 | 0 |
| NT2RP7002449 | 0 | 0 | 0 | 5.735 | 0 | 6.107 | 0 |
| NT2RP7002594 | 0 | 0 | 0 | 0 | 0 | 13.764 | 0 |
| NT2RP7002619 | 0 | 0 | 0 | 0.773 | 0 | 1.098 | 0 |
| NT2RP7002738 | 0 | 0 | 0 | 4.086 | 0 | 0 | 0 |
| NT2RP7002802 | 0 | 0 | 0 | 2.594 | 0 | 0 | 0 |
| NT2RP7002829 | 0 | 0 | 0 | 6.206 | 0 | 0 | 0 |
| NT2RP7002875 | 0 | 0 | 0 | 3.930 | 3.334 | 6.508 | 0 |
| NT2RP7002906 | 0 | 0 | 0 | 3.980 | 0 | 8.475 | 0 |
| NT2RP7003055 | 0 | 0 | 0 | 0 | 0 | 10.632 | 0 |
| NT2RP7003203 | 0 | 0 | 0 | 2.622 | 0 | 0 | 0 |
| NT2RP7003319 | 0 | 0 | 0 | 1.943 | 3.297 | 0.919 | 0 |
| NT2RP7003511 | 0 | 0 | 25.483 | 0 | 0 | 0 | 0 |
| NT2RP7003632 | 0 | 0 | 0 | 3.467 | 0 | 4.922 | 0 |
| NT2RP7003647 | 0 | 0 | 0 | 8.353 | 0 | 4.743 | 0 |
| NT2RP7003688 | 0 | 0 | 0 | 4.193 | 0 | 6.696 | 0 |
| NT2RP7003724 | 0 | 0 | 0 | 26.217 | 0 | 0 | 0 |
| NT2RP7003960 | 0 | 0 | 0 | 2.352 | 0 | 0 | 0 |
| NT2RP7004114 | 0 | 0 | 0 | 7.915 | 0 | 0 | 0 |
| NT2RP7004123 | 0 | 13.469 | 0 | 1.362 | 0 | 0 | 0 |
| NT2RP7004173 | 0 | 0 | 0 | 2.710 | 2.760 | 3.078 | 0 |
| NT2RP7004196 | 0 | 0 | 0 | 3.720 | 4.734 | 5.281 | 0 |
| NT2RP7004352 | 0 | 0 | 0 | 4.822 | 0 | 1.711 | 0 |
| NT2RP7004373 | 0 | 0 | 0 | 1.671 | 0 | 3.558 | 0 |
| NT2RP7004428 | 0 | 0 | 0 | 7.181 | 0 | 6.796 | 0 |
| NT2RP7004481 | 0 | 0 | 0 | 3.817 | 0 | 0 | 0 |
| NT2RP7004728 | 0 | 0 | 0 | 2.231 | 0 | 4.751 | 0 |
| NT2RP7004751 | 0 | 0 | 0 | 0 | 1.691 | 0.943 | 0 |
| NT2RP7004790 | 0 | 0 | 0 | 11.078 | 0 | 0 | 0 |
| NT2RP7004884 | 0 | 0 | 0 | 11.691 | 0 | 2.766 | 0 |
| NT2RP7004911 | 0 | 0 | 0 | 1.251 | 0 | 0 | 0 |
| NT2RP7004915 | 0 | 0 | 0 | 10.538 | 0 | 7.480 | 0 |
| NT2RP7004925 | 0 | 0 | 0 | 4.481 | 0 | 1.272 | 0 |
| NT2RP7004946 | 0 | 0 | 0 | 3.666 | 0 | 0 | 0 |
| NT2RP7005219 | 0 | 0 | 0 | 37.067 | 0 | 0 | 0 |
| NT2RP7005493 | 0 | 0 | 0 | 0 | 43.725 | 0 | 0 |
| NT2RP7005513 | 0 | 0 | 0 | 0 | 0 | 1.703 | 0 |
| NT2RP7005529 | 0 | 0 | 0 | 5.240 | 0 | 7.439 | 0 |
| NT2RP7005675 | 0 | 0 | 0 | 8.256 | 0 | 5.861 | 0 |
| NT2RP7005750 | 0 | 0 | 0 | 5.079 | 0 | 0 | 0 |
| NT2RP7006162 | 0 | 0 | 0 | 22.750 | 0 | 0 | 0 |
| NT2RP7006188 | 0 | 0 | 0 | 0 | 0 | 2.623 | 0 |
| NT2RP7006223 | 0 | 0 | 0 | 17.994 | 0 | 0 | 0 |
| NT2RP7006263 | 0 | 0 | 31.053 | 8.574 | 0 | 0 | 0 |
| NT2RP7006490 | 0 | 0 | 0 | 1.523 | 0 | 0 | 0 |
| NT2RP7006619 | 0 | 0 | 0 | 1.567 | 7.976 | 0 | 0 |
| NT2RP7006701 | 0 | 0 | 38.773 | 7.137 | 0 | 5.066 | 0 |
| NT2RP7006717 | 0 | 0 | 10.147 | 1.401 | 0 | 0 | 0 |
| NT2RP7007154 | 0 | 0 | 0 | 0.809 | 0 | 0 | 0 |
| NT2RP7007177 | 0 | 0 | 0 | 0 | 0 | 2.744 | 0 |
| NT2RP7007252 | 0 | 0 | 0 | 9.748 | 0 | 6.919 | 0 |
| NT2RP7007530 | 0 | 0 | 0 | 1.556 | 0 | 2.209 | 0 |
| NT2RP7007537 | 0 | 8.631 | 0 | 5.236 | 0 | 1.239 | 0 |
| NT2RP7007610 | 0 | 0 | 0 | 6.162 | 0 | 0 | 0 |
| NT2RP7007842 | 0 | 0 | 0 | 4.084 | 6.930 | 0 | 0 |
| NT2RP7007975 | 0 | 0 | 0 | 21.309 | 0 | 1.375 | 0 |
| NT2RP7008161 | 0 | 0 | 0 | 4.456 | 0 | 0 | 0 |
| NT2RP7008167 | 0 | 0 | 0 | 1.288 | 0 | 0 | 0 |
| NT2RP7008190 | 0 | 9.311 | 0 | 0.941 | 0 | 0 | 0 |
| NT2RP7008396 | 0 | 0 | 0 | 0 | 0 | 12.403 | 0 |
| NT2RP7008441 | 0 | 0 | 0 | 14.289 | 0 | 0 | 0 |
| NT2RP7008543 | 0 | 19.310 | 0 | 0 | 0 | 2.772 | 0 |
| NT2RP7008714 | 0 | 0 | 0 | 8.258 | 0 | 23.445 | 0 |
| NT2RP7009012 | 0 | 0 | 0 | 8.047 | 0 | 0 | 0 |
| NT2RP7009087 | 0 | 0 | 0 | 1.724 | 0 | 2.448 | 0 |
| NT2RP7009097 | 0 | 0 | 0 | 0 | 6.496 | 0 | 0 |
| NT2RP7009108 | 0 | 0 | 0 | 0.631 | 12.846 | 8.060 | 0 |
| NT2RP7009149 | 0 | 0 | 0 | 0 | 4.323 | 1.205 | 0 |
| NT2RP7009259 | 0 | 0 | 4.333 | 2.393 | 0 | 4.529 | 0 |
| NT2RP7009373 | 0 | 0 | 0 | 8.554 | 0 | 0 | 0 |
| NT2RP7009394 | 0 | 0 | 0 | 0.858 | 4.369 | 1.218 | 0 |
| NT2RP7009481 | 0 | 0 | 0 | 2.907 | 0 | 4.127 | 0 |
| NT2RP7009482 | 0 | 0 | 0 | 2.631 | 0 | 0.467 | 0 |
| NT2RP7009498 | 0 | 0 | 0 | 0 | 0 | 1.015 | 0 |
| NT2RP7010109 | 0 | 0 | 0 | 11.153 | 0 | 0 | 0 |
| NT2RP7010599 | 0 | 0 | 0 | 22.750 | 0 | 0 | 0 |
| NT2RP7013573 | 0 | 0 | 0 | 5.114 | 0 | 0 | 0 |
| NT2RP7014910 | 0 | 0 | 0 | 0.849 | 0 | 1.205 | 0 |
| NT2RP7015503 | 0 | 0 | 0 | 2.247 | 0 | 0 | 0 |
| NT2RP7016574 | 0 | 0 | 0 | 0 | 0 | 22.259 | 0 |
| NT2RP7017474 | 0 | 0 | 0 | 13.360 | 0 | 0 | 0 |
| NT2RP7019543 | 0 | 0 | 0 | 0 | 38.221 | 0 | 0 |
| NT2RP8000521 | 0 | 0 | 0 | 37.067 | 0 | 0 | 0 |
| NT2RP8007715 | 0 | 0 | 0 | 44.589 | 0 | 0 | 0 |
| NT2RP8007920 | 0 | 0 | 0 | 1.404 | 0 | 0 | 0 |
| NTONG1000033 | 0 | 0 | 0 | 3.478 | 0 | 0 | 0 |
| NTONG1000098 | 0 | 0 | 0 | 4.070 | 0 | 1.284 | 0 |
| NTONG1000182 | 0 | 0 | 0 | 0 | 0 | 1.247 | 0 |
| NTONG1000246 | 0 | 0 | 12.058 | 0 | 0 | 0 | 0 |
| NTONG1000264 | 0 | 0 | 0 | 0 | 0 | 6.874 | 0 |
| NTONG2000107 | 0 | 0 | 0 | 0 | 0 | 10.745 | 0 |
| NTONG2000265 | 0 | 0 | 6.574 | 6.656 | 0 | 1.718 | 0 |
| NTONG2000334 | 0 | 0 | 0 | 1.451 | 7.388 | 0 | 0 |
| NTONG2000876 | 0 | 0 | 21.476 | 0 | 0 | 1.403 | 0 |
| NTONG2001428 | 0 | 0 | 0 | 0 | 0 | 10.621 | 0 |
| NTONG2001550 | 0 | 0 | 0 | 2.470 | 0 | 0 | 0 |
| NTONG2001612 | 0 | 7.879 | 0 | 0.797 | 0 | 0 | 0 |
| NTONG2001657 | 0 | 0 | 51.133 | 0 | 0 | 0 | 0 |
| NTONG2001676 | 0 | 0 | 0 | 5.063 | 0 | 0 | 0 |
| NTONG2001762 | 0 | 0 | 0 | 2.560 | 0 | 9.085 | 0 |
| NTONG2003454 | 0 | 0 | 0 | 0 | 0 | 3.004 | 0 |
| NTONG2004844 | 0 | 11.375 | 12.498 | 1.150 | 0 | 1.633 | 0 |
| NTONG2005391 | 0 | 0 | 0 | 1.655 | 0 | 4.698 | 0 |
| NTONG2005520 | 0 | 14.017 | 0 | 1.890 | 0 | 1.341 | 0 |
| NTONG2005801 | 0 | 0 | 0 | 3.793 | 0 | 1.795 | 0 |
| NTONG2005969 | 0 | 0 | 0 | 4.560 | 11.607 | 6.473 | 0 |
| NTONG2006484 | 0 | 0 | 0 | 3.068 | 0 | 0 | 0 |
| NTONG2006783 | 0 | 0 | 0 | 0 | 0 | 2.853 | 0 |
| NTONG2007658 | 0 | 0 | 0 | 4.261 | 0 | 0 | 0 |
| NTONG2007756 | 0 | 0 | 0 | 4.791 | 0 | 0 | 0 |
| NTONG2009468 | 0 | 0 | 0 | 13.482 | 0 | 0 | 0 |
| NTONG2009576 | 0 | 0 | 0 | 10.086 | 0 | 7.159 | 0 |
| OCBBF1000049 | 0 | 0 | 0 | 0 | 0 | 5.007 | 0 |
| OCBBF1000086 | 0 | 0 | 0 | 2.173 | 0 | 0 | 0 |
| OCBBF1000114 | 0 | 0 | 0 | 0 | 0 | 2.569 | 0 |
| OCBBF1000146 | 0 | 0 | 0 | 0 | 0 | 0.869 | 0 |
| OCBBF1000175 | 0 | 0 | 0 | 5.112 | 0 | 7.258 | 0 |
| OCBBF1000188 | 32.641 | 0 | 0 | 0.739 | 0 | 1.049 | 0 |
| OCBBF1000254 | 0 | 0 | 0 | 0 | 0 | 44.784 | 0 |
| OCBBF2000015 | 0 | 0 | 0 | 5.615 | 0 | 3.986 | 0 |
| OCBBF2000126 | 0 | 0 | 0 | 2.759 | 0 | 3.916 | 0 |
| OCBBF2000178 | 0 | 0 | 0 | 3.095 | 3.940 | 6.591 | 0 |
| OCBBF2000231 | 0 | 0 | 0 | 0 | 0 | 3.947 | 0 |
| OCBBF2000277 | 0 | 0 | 0 | 10.429 | 0 | 7.403 | 0 |
| OCBBF2000323 | 0 | 0 | 0 | 0 | 0 | 15.289 | 0 |
| OCBBF2000467 | 0 | 14.157 | 0 | 0 | 0 | 2.032 | 0 |
| OCBBF2000522 | 0 | 0 | 0 | 0 | 0 | 5.096 | 0 |
| OCBBF2000677 | 0 | 0 | 0 | 0 | 0 | 16.413 | 0 |
| OCBBF2000712 | 0 | 0 | 0 | 0.671 | 0 | 0 | 0 |
| OCBBF2000824 | 0 | 0 | 0 | 0 | 0 | 6.055 | 0 |
| OCBBF2000831 | 0 | 0 | 0 | 0 | 0 | 9.702 | 0 |
| OCBBF2000846 | 0 | 0 | 0 | 1.039 | 0 | 0.737 | 0 |
| OCBBF2000982 | 0 | 0 | 0 | 1.238 | 0 | 0.879 | 0 |
| OCBBF2000986 | 0 | 0 | 0 | 0.684 | 0 | 0.971 | 0 |
| OCBBF2001140 | 0 | 0 | 0 | 0 | 0 | 11.393 | 0 |
| OCBBF2001166 | 0 | 0 | 0 | 15.560 | 0 | 0 | 0 |
| OCBBF2001186 | 0 | 0 | 0 | 0 | 0 | 17.837 | 0 |
| OCBBF2001196 | 0 | 0 | 0 | 11.240 | 0 | 0 | 0 |
| OCBBF2001210 | 0 | 0 | 0 | 0 | 0 | 14.613 | 0 |
| OCBBF2001307 | 0 | 0 | 0 | 2.361 | 0 | 10.055 | 0 |
| OCBBF2001492 | 0 | 0 | 0 | 4.383 | 0 | 3.457 | 0 |
| OCBBF2001639 | 0 | 0 | 0 | 6.675 | 0 | 0 | 0 |
| OCBBF2001687 | 0 | 0 | 0 | 0 | 0 | 1.938 | 0 |
| OCBBF2001794 | 0 | 0 | 0 | 0 | 0 | 4.764 | 0 |
| OCBBF2001931 | 0 | 0 | 0 | 0 | 0 | 16.872 | 0 |
| OCBBF2002015 | 0 | 0 | 0 | 9.817 | 0 | 0 | 0 |
| OCBBF2002086 | 0 | 0 | 0 | 4.573 | 0 | 0 | 0 |
| OCBBF2002290 | 0 | 0 | 0 | 0 | 0 | 20.324 | 0 |
| OCBBF2002376 | 0 | 0 | 0 | 0 | 0 | 7.671 | 0 |
| OCBBF2002626 | 0 | 0 | 0 | 2.398 | 0 | 1.702 | 0 |
| OCBBF2002656 | 0 | 11.610 | 0 | 7.044 | 0 | 3.333 | 0 |
| OCBBF2003052 | 0 | 0 | 0 | 0 | 0 | 7.654 | 0 |
| OCBBF2003246 | 0 | 0 | 0 | 0 | 0 | 9.861 | 0 |
| OCBBF2003518 | 0 | 0 | 0 | 0 | 0 | 1.039 | 0 |
| OCBBF2003819 | 0 | 0 | 0 | 14.955 | 0 | 0 | 0 |
| OCBBF2003925 | 0 | 0 | 0 | 0 | 3.011 | 1.679 | 0 |
| OCBBF2004104 | 0 | 0 | 0 | 0 | 4.949 | 4.140 | 0 |
| OCBBF2004235 | 0 | 0 | 0 | 0 | 43.096 | 0 | 0 |
| OCBBF2004273 | 0 | 0 | 0 | 1.909 | 0 | 0 | 0 |
| OCBBF2004757 | 0 | 0 | 0 | 3.518 | 8.955 | 2.497 | 0 |
| OCBBF2005476 | 0 | 0 | 0 | 0 | 0 | 4.710 | 0 |
| OCBBF2005956 | 0 | 0 | 0 | 6.045 | 10.258 | 5.721 | 0 |
| OCBBF2006113 | 0 | 0 | 0 | 18.680 | 0 | 0 | 0 |
| OCBBF2006151 | 0 | 0 | 0 | 4.092 | 0 | 0 | 0 |
| OCBBF2006172 | 0 | 0 | 0 | 0 | 0 | 5.283 | 0 |
| OCBBF2006313 | 0 | 0 | 0 | 8.105 | 0 | 0 | 0 |
| OCBBF2006764 | 0 | 0 | 0 | 21.782 | 0 | 15.461 | 0 |
| OCBBF2007028 | 0 | 0 | 0 | 8.820 | 0 | 6.956 | 0 |
| OCBBF2007114 | 0 | 0 | 0 | 0 | 38.623 | 21.540 | 0 |
| OCBBF2007224 | 0 | 0 | 0 | 6.702 | 0 | 0 | 0 |
| OCBBF2007354 | 31.490 | 0 | 0 | 0.713 | 0 | 1.012 | 0 |
| OCBBF2007415 | 0 | 0 | 0 | 3.410 | 0 | 4.841 | 0 |
| OCBBF2007622 | 0 | 0 | 0 | 2.606 | 0 | 0 | 0 |
| OCBBF2007762 | 0 | 0 | 0 | 0 | 0 | 15.843 | 0 |
| OCBBF2007829 | 0 | 0 | 0 | 5.846 | 0 | 1.106 | 0 |
| OCBBF2008005 | 0 | 11.145 | 0 | 6.762 | 0 | 6.399 | 0 |
| OCBBF2008144 | 0 | 0 | 0 | 4.094 | 0 | 2.906 | 0 |
| OCBBF2008340 | 0 | 0 | 0 | 0 | 0 | 7.778 | 0 |
| OCBBF2008511 | 0 | 0 | 0 | 7.836 | 39.893 | 11.124 | 0 |
| OCBBF2008701 | 0 | 0 | 0 | 0 | 0 | 10.920 | 0 |
| OCBBF2008775 | 0 | 0 | 0 | 0 | 0 | 44.784 | 0 |
| OCBBF2009095 | 0 | 0 | 0 | 4.665 | 23.748 | 6.622 | 0 |
| OCBBF2009242 | 0 | 0 | 0 | 6.029 | 10.231 | 0 | 0 |
| OCBBF2009301 | 0 | 0 | 0 | 0 | 0 | 4.177 | 0 |
| OCBBF2009352 | 0 | 0 | 0 | 0 | 40.679 | 28.358 | 0 |
| OCBBF2009772 | 0 | 0 | 0 | 6.686 | 0 | 7.119 | 0 |
| OCBBF2009836 | 0 | 0 | 0 | 8.725 | 0 | 4.129 | 0 |
| OCBBF2010313 | 0 | 0 | 0 | 0 | 49.562 | 0 | 0 |
| OCBBF2010557 | 0 | 0 | 0 | 13.005 | 0 | 0 | 0 |
| OCBBF2010819 | 0 | 0 | 0 | 8.903 | 0 | 0 | 0 |
| OCBBF2010858 | 0 | 0 | 0 | 4.728 | 0 | 0 | 0 |
| OCBBF2010863 | 0 | 0 | 0 | 0 | 0 | 44.784 | 0 |
| OCBBF2010871 | 0 | 0 | 0 | 3.244 | 0 | 13.817 | 0 |
| OCBBF2011021 | 0 | 0 | 0 | 6.155 | 0 | 8.738 | 0 |
| OCBBF2011073 | 0 | 0 | 0 | 26.641 | 0 | 0 | 0 |
| OCBBF2011232 | 0 | 0 | 0 | 5.687 | 0 | 8.073 | 0 |
| OCBBF2011536 | 0 | 0 | 0 | 2.832 | 0 | 2.010 | 0 |
| OCBBF2011669 | 0 | 0 | 0 | 36.360 | 0 | 0 | 0 |
| OCBBF2011759 | 0 | 0 | 0 | 8.623 | 0 | 2.825 | 0 |
| OCBBF2011887 | 0 | 0 | 0 | 6.676 | 0 | 0 | 0 |
| OCBBF2011897 | 0 | 0 | 0 | 0 | 0 | 17.395 | 0 |
| OCBBF2011914 | 0 | 0 | 0 | 2.588 | 13.175 | 3.674 | 0 |
| OCBBF2012039 | 0 | 0 | 0 | 3.266 | 0 | 7.727 | 0 |
| OCBBF2012525 | 0 | 0 | 0 | 15.998 | 0 | 0 | 0 |
| OCBBF2012553 | 0 | 0 | 0 | 0 | 4.238 | 1.182 | 0 |
| OCBBF2012678 | 0 | 0 | 0 | 29.374 | 0 | 0 | 0 |
| OCBBF2012704 | 0 | 0 | 0 | 14.819 | 0 | 0 | 0 |
| OCBBF2012714 | 0 | 0 | 0 | 4.120 | 0 | 0 | 0 |
| OCBBF2012812 | 0 | 0 | 0 | 0 | 74.416 | 0 | 0 |
| OCBBF2012821 | 0 | 0 | 0 | 0 | 0 | 2.521 | 0 |
| OCBBF2012881 | 0 | 0 | 0 | 0 | 0 | 44.784 | 0 |
| OCBBF2012908 | 0 | 0 | 0 | 0 | 0 | 9.905 | 0 |
| OCBBF2013011 | 0 | 0 | 0 | 0 | 0 | 22.823 | 0 |
| OCBBF2013149 | 0 | 10.886 | 0 | 3.303 | 5.604 | 14.065 | 0 |
| OCBBF2013285 | 0 | 0 | 0 | 36.360 | 0 | 0 | 0 |
| OCBBF2013324 | 0 | 0 | 0 | 53.329 | 0 | 0 | 0 |
| OCBBF2013366 | 0 | 0 | 0 | 1.916 | 0 | 0 | 0 |
| OCBBF2013481 | 0 | 0 | 0 | 3.880 | 0 | 5.509 | 0 |
| OCBBF2013843 | 0 | 0 | 0 | 3.413 | 0 | 2.154 | 0 |
| OCBBF2014064 | 0 | 0 | 0 | 0 | 0 | 1.376 | 0 |
| OCBBF2014089 | 0 | 0 | 0 | 0 | 0 | 19.885 | 0 |
| OCBBF2014292 | 0 | 0 | 0 | 0 | 0 | 1.044 | 0 |
| OCBBF2014576 | 0 | 0 | 0 | 2.593 | 0 | 0 | 0 |
| OCBBF2014745 | 0 | 0 | 0 | 36.360 | 0 | 0 | 0 |
| OCBBF2015115 | 0 | 0 | 0 | 12.664 | 0 | 0 | 0 |
| OCBBF2015233 | 0 | 0 | 0 | 22.219 | 0 | 0 | 0 |
| OCBBF2015751 | 0 | 0 | 0 | 0 | 0 | 8.566 | 0 |
| OCBBF2016689 | 0 | 0 | 0 | 22.219 | 0 | 0 | 0 |
| OCBBF2016729 | 0 | 0 | 0 | 0 | 0 | 27.555 | 0 |
| OCBBF2017035 | 0 | 17.018 | 0 | 2.581 | 13.142 | 1.222 | 0 |
| OCBBF2017055 | 0 | 0 | 0 | 12.735 | 0 | 18.079 | 0 |
| OCBBF2017398 | 0 | 9.029 | 0 | 1.826 | 0 | 3.888 | 0 |
| OCBBF2017458 | 0 | 0 | 0 | 7.903 | 0 | 5.609 | 0 |
| OCBBF2017754 | 0 | 0 | 0 | 7.995 | 0 | 0 | 0 |
| OCBBF2017791 | 0 | 0 | 0 | 10 | 0 | 3.872 | 0 |
| OCBBF2017882 | 0 | 0 | 0 | 0 | 0 | 7.189 | 0 |
| OCBBF2017893 | 0 | 12.175 | 0 | 0 | 0 | 0 | 0 |
| OCBBF2018012 | 0 | 0 | 0 | 0 | 0 | 2.828 | 0 |
| OCBBF2018084 | 0 | 30.950 | 0 | 9.389 | 0 | 4.443 | 0 |
| OCBBF2018234 | 0 | 0 | 0 | 9.194 | 0 | 19.579 | 0 |
| OCBBF2018581 | 0 | 30.197 | 0 | 0 | 0 | 8.670 | 0 |
| OCBBF2018618 | 0 | 0 | 0 | 0 | 0 | 16.341 | 0 |
| OCBBF2018707 | 0 | 0 | 0 | 1.596 | 16.252 | 2.266 | 0 |
| OCBBF2018827 | 0 | 0 | 0 | 3.483 | 0 | 4.944 | 0 |
| OCBBF2018956 | 0 | 0 | 0 | 3.604 | 0 | 1.705 | 0 |
| OCBBF2020343 | 0 | 0 | 0 | 36.360 | 0 | 0 | 0 |
| OCBBF2020741 | 0 | 0 | 0 | 20.028 | 0 | 0 | 0 |
| OCBBF2021286 | 0 | 0 | 0 | 6.550 | 0 | 0 | 0 |
| OCBBF2022351 | 0 | 0 | 0 | 36.360 | 0 | 0 | 0 |
| OCBBF2023162 | 0 | 0 | 0 | 0 | 37.152 | 31.080 | 0 |
| OCBBF2024850 | 0 | 0 | 0 | 1.577 | 0 | 2.239 | 0 |
| OCBBF2025527 | 0 | 0 | 0 | 36.360 | 0 | 0 | 0 |
| OCBBF2026368 | 0 | 0 | 0 | 5.865 | 0 | 8.326 | 0 |
| OCBBF2026649 | 0 | 0 | 0 | 36.360 | 0 | 0 | 0 |
| OCBBF2027197 | 0 | 0 | 0 | 36.360 | 0 | 0 | 0 |
| OCBBF2027661 | 0 | 0 | 0 | 53.329 | 0 | 0 | 0 |
| OCBBF2030329 | 0 | 0 | 0 | 0 | 0 | 44.784 | 0 |
| OCBBF2030927 | 0 | 0 | 0 | 0 | 0 | 44.784 | 0 |
| OCBBF2033295 | 0 | 0 | 0 | 7.464 | 0 | 0 | 0 |
| OCBBF2035214 | 0 | 0 | 0 | 0 | 0 | 44.784 | 0 |
| OCBBF2035226 | 0 | 0 | 0 | 0.417 | 0 | 0 | 0 |
| OCBBF2036743 | 0 | 0 | 0 | 15.690 | 0 | 11.137 | 0 |
| OCBBF3000323 | 0 | 0 | 0 | 7.082 | 0 | 3.351 | 0 |
| OCBBF3001333 | 0 | 0 | 0 | 4.561 | 0 | 6.475 | 0 |
| OCBBF3004487 | 0 | 0 | 0 | 6.432 | 0 | 4.565 | 0 |
| OCBBF3009244 | 0 | 0 | 0 | 0 | 0 | 34.702 | 0 |
| PANCR1000091 | 0 | 0 | 0 | 0.214 | 0 | 0 | 0 |
| PEBLM1000174 | 0 | 0 | 0 | 3.394 | 0 | 0 | 0 |
| PEBLM2000147 | 0 | 0 | 0 | 1.811 | 0 | 10.283 | 0 |
| PEBLM2000222 | 0 | 0 | 0 | 0 | 0 | 0.585 | 0 |
| PEBLM2000267 | 0 | 0 | 0 | 1.996 | 25.404 | 2.834 | 0 |
| PEBLM2000326 | 0 | 0 | 0 | 7.705 | 0 | 0 | 0 |
| PEBLM2001072 | 0 | 0 | 0 | 5.148 | 0 | 1.827 | 0 |
| PEBLM2003914 | 0 | 14.913 | 0 | 3.016 | 7.677 | 4.282 | 0 |
| PEBLM2004452 | 0 | 0 | 0 | 1.916 | 0 | 2.720 | 0 |
| PEBLM2004666 | 0 | 0 | 0 | 0 | 0 | 3.744 | 0 |
| PEBLM2005282 | 0 | 0 | 0 | 0 | 0 | 11.820 | 0 |
| PEBLM2007437 | 0 | 0 | 0 | 0 | 21.543 | 0 | 0 |
| PEBLM2007774 | 0 | 0 | 0 | 0.214 | 0 | 0 | 0 |
| PERIC2000478 | 0 | 0 | 0 | 7.727 | 0 | 0 | 0 |
| PERIC2000889 | 0 | 0 | 0 | 6.314 | 0 | 8.963 | 0 |
| PERIC2003720 | 0 | 0 | 0 | 0 | 0 | 13.012 | 0 |
| PERIC2005117 | 0 | 0 | 0 | 1.525 | 0 | 2.164 | 0 |
| PERIC2006443 | 0 | 34.088 | 0 | 0 | 0 | 0 | 0 |
| PERIC2007068 | 0 | 0 | 0 | 8.761 | 0 | 0 | 0 |
| PLACE5000066 | 0 | 0 | 0 | 0 | 0 | 18.438 | 0 |
| PLACE5000113 | 0 | 0 | 0 | 6.106 | 0 | 1.734 | 0 |
| PLACE5000159 | 0 | 0 | 0 | 0 | 0 | 2.290 | 0 |
| PLACE5000170 | 0 | 0 | 0 | 0 | 0 | 3.503 | 0 |
| PLACE5000172 | 0 | 0 | 0 | 4.613 | 0 | 2.619 | 0 |
| PLACE6000145 | 0 | 7.274 | 0 | 0 | 0 | 0 | 0 |
| PLACE6000263 | 0 | 0 | 11.725 | 2.158 | 0 | 4.596 | 0 |
| PLACE6000379 | 0 | 2.479 | 1.362 | 1.003 | 0.638 | 0.712 | 0 |
| PLACE6000414 | 0 | 0 | 9.882 | 0 | 0 | 0 | 0 |
| PLACE6000619 | 0 | 0 | 0 | 0 | 0 | 36.150 | 0 |
| PLACE6001064 | 0 | 0 | 0 | 4.479 | 2.073 | 1.156 | 0 |
| PLACE6001185 | 0 | 0 | 0 | 15.159 | 0 | 0 | 0 |
| PLACE6001262 | 0 | 0 | 0 | 6.202 | 0 | 0 | 0 |
| PLACE6001281 | 0 | 0 | 0 | 2.443 | 0 | 0 | 0 |
| PLACE6001443 | 0 | 0 | 15.148 | 0 | 0 | 0 | 0 |
| PLACE6001823 | 0 | 0 | 0 | 20.223 | 0 | 0 | 0 |
| PLACE6002084 | 0 | 0 | 2.848 | 0.262 | 0 | 1.116 | 0 |
| PLACE6002102 | 0 | 0 | 0 | 2.005 | 0 | 1.423 | 0 |
| PLACE6002419 | 0 | 0 | 0 | 0 | 0 | 10.504 | 0 |
| PLACE6002692 | 0 | 0 | 0 | 0 | 0 | 36.150 | 0 |
| PLACE6003038 | 0 | 0 | 0 | 2.561 | 0 | 0 | 0 |
| PLACE6003621 | 0 | 0 | 0 | 28.511 | 0 | 0 | 0 |
| PLACE6004005 | 0 | 0 | 0 | 7.708 | 3.568 | 3.979 | 0 |
| PLACE6004312 | 0 | 0 | 0 | 0.856 | 0 | 1.823 | 0 |
| PLACE6004397 | 0 | 0 | 0 | 1.305 | 13.283 | 1.852 | 0 |
| PLACE6004578 | 0 | 0 | 0 | 1.760 | 0 | 1.249 | 0 |
| PLACE6005231 | 0 | 0 | 0 | 31.356 | 0 | 0 | 0 |
| PLACE6005482 | 0 | 0 | 0 | 0 | 0 | 2.664 | 0 |
| PLACE6005691 | 0 | 0 | 0 | 0 | 0 | 36.150 | 0 |
| PLACE6006137 | 0 | 0 | 0 | 2.424 | 12.342 | 6.883 | 0 |
| PLACE6006549 | 0 | 5.605 | 0 | 0.567 | 0 | 0 | 0 |
| PLACE6007180 | 0 | 0 | 0 | 15.213 | 0 | 32.395 | 0 |
| PLACE6007373 | 89.702 | 0 | 0 | 2.031 | 0 | 0 | 0 |
| PLACE6007436 | 0 | 0 | 0 | 5.100 | 0 | 7.240 | 0 |
| PLACE6008444 | 0 | 0 | 0 | 0 | 0 | 36.150 | 0 |
| PLACE6008775 | 0 | 0 | 0 | 5.233 | 0 | 14.857 | 0 |
| PLACE6009338 | 0 | 0 | 0 | 0 | 0 | 25.317 | 0 |
| PLACE6010484 | 0 | 10.849 | 0 | 1.097 | 0 | 3.115 | 0 |
| PLACE6010936 | 0 | 0 | 0 | 0 | 0 | 12.399 | 0 |
| PLACE6011041 | 0 | 0 | 0 | 1.931 | 0 | 0 | 0 |
| PLACE6011057 | 0 | 0 | 0 | 1.600 | 8.144 | 0 | 0 |
| PLACE6012028 | 0 | 0 | 0 | 0 | 0 | 12.650 | 0 |
| PLACE6013672 | 0 | 0 | 0 | 0 | 0 | 27.825 | 0 |
| PLACE6014064 | 0 | 21.482 | 0 | 0 | 0 | 3.084 | 0 |
| PLACE6015051 | 0 | 32.356 | 0 | 0 | 0 | 0 | 0 |
| PLACE6015445 | 0 | 0 | 0 | 0 | 40.154 | 0 | 0 |
| PLACE6015729 | 0 | 0 | 0 | 0 | 0 | 36.150 | 0 |
| PLACE6016942 | 0 | 0 | 0 | 0 | 0 | 3.942 | 0 |
| PLACE6018031 | 0 | 0 | 0 | 4.772 | 0 | 0 | 0 |
| PLACE6019195 | 0 | 0 | 0 | 6.023 | 0 | 0 | 0 |
| PLACE7004103 | 0 | 0 | 0 | 0.918 | 0 | 1.303 | 0 |
| PLACE7008261 | 0 | 0 | 0 | 20.223 | 0 | 0 | 0 |
| PROST1000097 | 0 | 4.127 | 0 | 3.339 | 2.125 | 1.185 | 0 |
| PROST1000199 | 0 | 5.128 | 0 | 0.519 | 0 | 0 | 0 |
| PROST1000215 | 0 | 0 | 0 | 3.630 | 0 | 5.154 | 0 |
| PROST1000220 | 0 | 0 | 0 | 0.482 | 0 | 0 | 0 |
| PROST1000226 | 0 | 0 | 27.595 | 0 | 0 | 0 | 0 |
| PROST1000272 | 0 | 0 | 0 | 0 | 0 | 3.179 | 0 |
| PROST1000526 | 0 | 0 | 0 | 1.793 | 0 | 2.546 | 0 |
| PROST1000564 | 0 | 0 | 0 | 0.954 | 0 | 0 | 0 |
| PROST2000138 | 0 | 1.825 | 0 | 0.185 | 0 | 0.611 | 0 |
| PROST2000176 | 0 | 17.815 | 0 | 0 | 0 | 0 | 0 |
| PROST2000284 | 0 | 0 | 0 | 4.971 | 12.653 | 0 | 0 |
| PROST2000567 | 0 | 0 | 0 | 0 | 0 | 14.432 | 0 |
| PROST2000761 | 0 | 66.434 | 0 | 0 | 0 | 0 | 0 |
| PROST2001002 | 0 | 0 | 0 | 13.944 | 0 | 0 | 0 |
| PROST2001180 | 0 | 0 | 0 | 7.049 | 0 | 2.502 | 0 |
| PROST2001283 | 0 | 0 | 0 | 0 | 7.688 | 2.144 | 0 |
| PROST2001521 | 0 | 8.528 | 0 | 0 | 0 | 0 | 0 |
| PROST2001883 | 0 | 0 | 0 | 0.634 | 0 | 0 | 0 |
| PROST2001997 | 0 | 0 | 0 | 12.414 | 0 | 0 | 0 |
| PROST2002162 | 0 | 0 | 0 | 0 | 0 | 3.901 | 0 |
| PROST2002227 | 0 | 17.841 | 0 | 0 | 0 | 0 | 0 |
| PROST2002425 | 0 | 0 | 0 | 0 | 0 | 16.010 | 0 |
| PROST2002651 | 0 | 0 | 0 | 0 | 10.806 | 0 | 0 |
| PROST2003232 | 0 | 0 | 0 | 4.843 | 0 | 2.750 | 0 |
| PROST2003338 | 0 | 7.856 | 0 | 3.972 | 4.044 | 1.128 | 0 |
| PROST2003396 | 0 | 0 | 0 | 4.240 | 0 | 6.020 | 0 |
| PROST2004095 | 0 | 0 | 0 | 8.969 | 0 | 2.830 | 0 |
| PROST2004270 | 0 | 0 | 0 | 0.835 | 0 | 2.370 | 0 |
| PROST2004416 | 21.314 | 0 | 0 | 0 | 0 | 0.685 | 0 |
| PROST2004570 | 0 | 0 | 0 | 5.320 | 27.085 | 0 | 0 |
| PROST2004739 | 0 | 0 | 0 | 10.556 | 0 | 0 | 0 |
| PROST2004744 | 0 | 37.106 | 0 | 0 | 0 | 0 | 0 |
| PROST2005067 | 0 | 0 | 0 | 11.450 | 0 | 0 | 0 |
| PROST2005093 | 0 | 0 | 0 | 16.676 | 0 | 0 | 0 |
| PROST2005143 | 0 | 0 | 0 | 4.815 | 0 | 0 | 0 |
| PROST2005285 | 0 | 0 | 0 | 5.298 | 0 | 0 | 0 |
| PROST2005466 | 0 | 0 | 0 | 16.676 | 0 | 0 | 0 |
| PROST2005604 | 0 | 0 | 0 | 2.208 | 0 | 0 | 0 |
| PROST2005683 | 0 | 0 | 0 | 6.457 | 0 | 0 | 0 |
| PROST2005793 | 0 | 27.525 | 0 | 0 | 0 | 0 | 0 |
| PROST2006030 | 0 | 0 | 0 | 13.428 | 0 | 0 | 0 |
| PROST2006060 | 0 | 0 | 0 | 9.478 | 0 | 0 | 0 |
| PROST2006260 | 0 | 0 | 0 | 5.780 | 0 | 0 | 0 |
| PROST2006737 | 0 | 0 | 0 | 0 | 8.758 | 4.884 | 0 |
| PROST2006988 | 0 | 0 | 0 | 0 | 0 | 14.713 | 0 |
| PROST2007248 | 0 | 0 | 0 | 13.229 | 0 | 0 | 0 |
| PROST2008271 | 0 | 0 | 0 | 11.126 | 0 | 3.949 | 0 |
| PROST2008360 | 0 | 0 | 0 | 2.555 | 0 | 0 | 0 |
| PROST2008489 | 0 | 0 | 4.994 | 0 | 0 | 0.653 | 0 |
| PROST2008770 | 0 | 0 | 0 | 7.197 | 0 | 3.930 | 0 |
| PROST2009347 | 0 | 0 | 0 | 13.452 | 0 | 0 | 0 |
| PROST2010382 | 0 | 0 | 0 | 0 | 0 | 3.153 | 0 |
| PROST2011105 | 0 | 0 | 0 | 25.260 | 0 | 0 | 0 |
| PROST2011410 | 0 | 0 | 0 | 0 | 0 | 3.995 | 0 |
| PROST2011577 | 0 | 0 | 0 | 5.843 | 0 | 0 | 0 |
| PROST2011660 | 0 | 0 | 0 | 16.676 | 0 | 0 | 0 |
| PROST2012007 | 0 | 0 | 0 | 0.806 | 4.101 | 0 | 0 |
| PROST2012016 | 0 | 0 | 0 | 0 | 0 | 22.126 | 0 |
| PROST2012745 | 0 | 0 | 0 | 0 | 0 | 7.793 | 0 |
| PROST2013121 | 0 | 0 | 0 | 7.634 | 0 | 0 | 0 |
| PROST2014862 | 0 | 0 | 0 | 0 | 0 | 16.010 | 0 |
| PROST2015332 | 0 | 0 | 0 | 27.441 | 0 | 2.597 | 0 |
| PROST2015900 | 0 | 0 | 0 | 16.676 | 0 | 0 | 0 |
| PROST2016351 | 0 | 0 | 0 | 0 | 0 | 8.925 | 0 |
| PROST2016918 | 0 | 0 | 0 | 4.511 | 22.963 | 0 | 0 |
| PROST2017128 | 0 | 0 | 0 | 0 | 0 | 8.119 | 0 |
| PROST2017203 | 0 | 0 | 0 | 9.217 | 0 | 0 | 0 |
| PROST2017529 | 0 | 0 | 0 | 1.802 | 0 | 0 | 0 |
| PROST2017692 | 23.099 | 0 | 0 | 0 | 0 | 0.742 | 0 |
| PROST2017910 | 0 | 0 | 0 | 0 | 37.758 | 0 | 0 |
| PROST2019164 | 0 | 25.881 | 0 | 0 | 0 | 0 | 0 |
| PROST2019253 | 0 | 0 | 0 | 0 | 0 | 1.693 | 0 |
| PROST2019296 | 0 | 79.832 | 0 | 0 | 0 | 0 | 0 |
| PUAEN1000057 | 0 | 0 | 0 | 1.457 | 0 | 0 | 0 |
| PUAEN1000065 | 0 | 0 | 0 | 2.377 | 2.420 | 0.675 | 0 |
| PUAEN1000147 | 0 | 0 | 0 | 0 | 0 | 9.395 | 0 |
| PUAEN1000164 | 0 | 0 | 3.070 | 0.848 | 0 | 0.401 | 0 |
| PUAEN1000322 | 0 | 0 | 0 | 0 | 9.239 | 0 | 0 |
| PUAEN2000152 | 0 | 0 | 0 | 0 | 0 | 5.980 | 0 |
| PUAEN2000374 | 0 | 0 | 0 | 0.909 | 1.156 | 0.322 | 0 |
| PUAEN2001586 | 0 | 0 | 0 | 2.027 | 0 | 0 | 0 |
| PUAEN2002489 | 0 | 0 | 0 | 9.262 | 0 | 0 | 0 |
| PUAEN2004400 | 0 | 0 | 0 | 0 | 0 | 7.259 | 0 |
| PUAEN2007044 | 0 | 0 | 0 | 5.087 | 0 | 1.444 | 0 |
| PUAEN2007898 | 0 | 0 | 0 | 0 | 39.038 | 0 | 0 |
| PUAEN2008515 | 0 | 0 | 0 | 13.781 | 0 | 0 | 0 |
| PUAEN2009174 | 0 | 0 | 0 | 0 | 0 | 2.856 | 0 |
| PUAEN2010824 | 0 | 0 | 0 | 11.155 | 0 | 26.393 | 0 |
| SALGL1000047 | 0 | 0 | 0 | 0.421 | 0 | 0.298 | 0 |
| SALGL1000157 | 0 | 0 | 0 | 0.185 | 0.940 | 0.655 | 0 |
| SKMUS1000022 | 0 | 0 | 0 | 0.886 | 0 | 0.839 | 0 |
| SKMUS1000084 | 0 | 0 | 0 | 2.186 | 0 | 1.552 | 0 |
| SKMUS1000110 | 0 | 0 | 0 | 5.229 | 0 | 7.423 | 0 |
| SKMUS1000177 | 0 | 0 | 0 | 0 | 0 | 2.379 | 0 |
| SKMUS1000186 | 0 | 0 | 0 | 0.855 | 0 | 2.022 | 0 |
| SKMUS2000020 | 0 | 25.852 | 0 | 0 | 0 | 0 | 0 |
| SKMUS2000074 | 0 | 0 | 0 | 0 | 18.480 | 0 | 0 |
| SKMUS2000117 | 0 | 0 | 0 | 0 | 30.703 | 0 | 0 |
| SKMUS2000243 | 0 | 0 | 0 | 7.160 | 0 | 0 | 0 |
| SKMUS2000271 | 0 | 0 | 0 | 9.055 | 0 | 0 | 0 |
| SKMUS2000343 | 0 | 0 | 0 | 10.937 | 0 | 7.763 | 0 |
| SKMUS2000361 | 0 | 7.506 | 0 | 0 | 0 | 1.077 | 0 |
| SKMUS2000380 | 0 | 0 | 0 | 1.539 | 0 | 0 | 0 |
| SKMUS2000484 | 0 | 0 | 0 | 13.710 | 6.345 | 3.539 | 0 |
| SKMUS2000873 | 0 | 0 | 0 | 0.390 | 0 | 0 | 0 |
| SKMUS2000902 | 0 | 0 | 0 | 0 | 0 | 5.450 | 0 |
| SKMUS2000931 | 0 | 0 | 0 | 1.931 | 0 | 0.548 | 0 |
| SKMUS2000954 | 0 | 0 | 0 | 1.206 | 0 | 3.425 | 0 |
| SKMUS2001008 | 0 | 0 | 0 | 0.336 | 0 | 0 | 0 |
| SKMUS2001129 | 0 | 0 | 0 | 3.609 | 0 | 5.124 | 0 |
| SKMUS2001201 | 0 | 0 | 0 | 0 | 0 | 1.795 | 0 |
| SKMUS2001254 | 0 | 0 | 0 | 0 | 0 | 12.511 | 0 |
| SKMUS2001402 | 0 | 0 | 0 | 2.992 | 0 | 0 | 0 |
| SKMUS2001412 | 0 | 0 | 0 | 0 | 0 | 12.511 | 0 |
| SKMUS2001454 | 0 | 0 | 0 | 0.958 | 1.220 | 2.380 | 0 |
| SKMUS2001501 | 0 | 0 | 0 | 3.094 | 0 | 0 | 0 |
| SKMUS2001528 | 0 | 0 | 0 | 0.891 | 13.606 | 0 | 0 |
| SKMUS2001543 | 0 | 0 | 0 | 23.206 | 0 | 0 | 0 |
| SKMUS2001634 | 0 | 0 | 0 | 0.929 | 4.731 | 1.319 | 0 |
| SKMUS2004908 | 0 | 0 | 0 | 9.325 | 0 | 6.619 | 0 |
| SKNMC1000168 | 0 | 0 | 6.458 | 0 | 0 | 0.844 | 0 |
| SKNMC1000251 | 0 | 0 | 0 | 4.978 | 0 | 0 | 0 |
| SKNMC1000264 | 0 | 36.814 | 0 | 0 | 0 | 5.285 | 0 |
| SKNMC2000065 | 0 | 0 | 0 | 3.831 | 0 | 1.088 | 0 |
| SKNMC2000097 | 0 | 0 | 6.874 | 1.898 | 0 | 0 | 0 |
| SKNMC2000224 | 0 | 0 | 0 | 0 | 0 | 6.311 | 0 |
| SKNMC2000305 | 0 | 0 | 0 | 2.500 | 0 | 10.647 | 0 |
| SKNMC2000322 | 0 | 0 | 0 | 2.005 | 0 | 0 | 0 |
| SKNMC2000635 | 5.623 | 1.259 | 2.767 | 0.127 | 0.648 | 0.181 | 0 |
| SKNMC2000649 | 0 | 0 | 0 | 1.295 | 3.297 | 0 | 0 |
| SKNMC2000698 | 0 | 0 | 0 | 0 | 0 | 2.081 | 0 |
| SKNMC2000877 | 0 | 0 | 0 | 0 | 0 | 3.582 | 0 |
| SKNMC2000966 | 0 | 0 | 0 | 0 | 0 | 8.159 | 0 |
| SKNMC2001113 | 0 | 0 | 0 | 6.928 | 0 | 0 | 0 |
| SKNMC2001503 | 0 | 0 | 0 | 0 | 0 | 6.249 | 0 |
| SKNMC2002402 | 0 | 0 | 0 | 0 | 0 | 7.428 | 0 |
| SKNSH1000002 | 0 | 10.089 | 0 | 1.020 | 0 | 1.448 | 0 |
| SKNSH1000101 | 0 | 0 | 0 | 0.912 | 2.321 | 0.647 | 0 |
| SKNSH2000101 | 0 | 0 | 0 | 4.677 | 0 | 6.640 | 0 |
| SKNSH2000151 | 0 | 0 | 0 | 15.445 | 0 | 5.482 | 0 |
| SKNSH2000163 | 0 | 0 | 0 | 7.532 | 0 | 0 | 0 |
| SKNSH2000250 | 0 | 0 | 0 | 8.000 | 0 | 8.517 | 0 |
| SKNSH2000319 | 0 | 0 | 0 | 0 | 25.913 | 0 | 0 |
| SKNSH2000347 | 0 | 0 | 0 | 0 | 0 | 2.261 | 0 |
| SKNSH2000482 | 0 | 0 | 0 | 0 | 0 | 9.821 | 0 |
| SKNSH2000819 | 0 | 0 | 0 | 1.767 | 8.994 | 12.539 | 0 |
| SKNSH2002325 | 0 | 0 | 0 | 5.419 | 0 | 0 | 0 |
| SKNSH2002768 | 0 | 0 | 0 | 1.680 | 0 | 0 | 0 |
| SKNSH2003064 | 0 | 0 | 0 | 0 | 28.287 | 0 | 0 |
| SKNSH2003528 | 0 | 0 | 0 | 0 | 0 | 3.059 | 0 |
| SKNSH2003633 | 0 | 0 | 0 | 0 | 0 | 6.759 | 0 |
| SKNSH2005061 | 0 | 0 | 0 | 3.610 | 0 | 1.708 | 0 |
| SKNSH2006234 | 0 | 0 | 0 | 0 | 0 | 3.527 | 0 |
| SMINT1000100 | 0 | 0 | 0 | 3.952 | 0 | 2.805 | 0 |
| SMINT1000117 | 0 | 0 | 10.143 | 1.867 | 0 | 1.325 | 0 |
| SMINT1000131 | 0 | 6.428 | 0 | 0 | 0 | 0 | 0 |
| SMINT1000192 | 0 | 0 | 22.965 | 0 | 0 | 0 | 0 |
| SMINT2000018 | 0 | 0 | 0 | 0 | 0 | 4.242 | 0 |
| SMINT2000073 | 0 | 0 | 0 | 6.157 | 0 | 0 | 0 |
| SMINT2000232 | 0 | 0 | 0 | 20.208 | 0 | 0 | 0 |
| SMINT2000277 | 0 | 12.725 | 0 | 0 | 0 | 0 | 0 |
| SMINT2000441 | 0 | 0 | 6.962 | 1.922 | 0 | 2.274 | 0 |
| SMINT2000454 | 0 | 0 | 0 | 6.343 | 0 | 0 | 0 |
| SMINT2000468 | 0 | 0 | 0 | 0 | 0 | 4.611 | 0 |
| SMINT2000602 | 0 | 0 | 0 | 0 | 0 | 1.108 | 0 |
| SMINT2000629 | 0 | 18.850 | 0 | 1.906 | 9.704 | 8.118 | 0 |
| SMINT2000659 | 0 | 0 | 0 | 0.197 | 0 | 0 | 0 |
| SMINT2000694 | 0 | 8.343 | 0 | 0.844 | 4.295 | 0 | 0 |
| SMINT2000933 | 0 | 7.153 | 0 | 0.723 | 0 | 0 | 0 |
| SMINT2001183 | 0 | 0 | 0 | 38.669 | 0 | 9.149 | 0 |
| SMINT2001818 | 0 | 0 | 0 | 0 | 3.387 | 0 | 0 |
| SMINT2002159 | 0 | 0 | 0 | 4.866 | 0 | 0 | 0 |
| SMINT2002689 | 0 | 0 | 0 | 10.012 | 0 | 1.184 | 0 |
| SMINT2003003 | 0 | 0 | 0 | 13.993 | 0 | 0 | 0 |
| SMINT2003150 | 0 | 0 | 0 | 0 | 0 | 22.212 | 0 |
| SMINT2003169 | 29.487 | 6.603 | 7.255 | 0 | 0 | 0.474 | 0 |
| SMINT2003340 | 0 | 0 | 0 | 0 | 0 | 2.960 | 0 |
| SMINT2004299 | 0 | 0 | 0 | 6.406 | 0 | 0 | 0 |
| SMINT2004350 | 0 | 0 | 0 | 0 | 0 | 26.332 | 0 |
| SMINT2004414 | 0 | 0 | 0 | 16.746 | 0 | 0 | 0 |
| SMINT2004583 | 0 | 0 | 0 | 0 | 0 | 3.903 | 0 |
| SMINT2004589 | 0 | 0 | 0 | 0 | 0 | 4.038 | 0 |
| SMINT2004872 | 0 | 0 | 0 | 6.004 | 0 | 0 | 0 |
| SMINT2004972 | 0 | 0 | 0 | 5.622 | 0 | 0 | 0 |
| SMINT2005368 | 0 | 0 | 0 | 1.667 | 4.243 | 0 | 0 |
| SMINT2005402 | 0 | 0 | 0 | 16.746 | 0 | 0 | 0 |
| SMINT2005956 | 0 | 18.474 | 0 | 0 | 0 | 0 | 0 |
| SMINT2007187 | 0 | 0 | 0 | 14.294 | 0 | 0 | 0 |
| SMINT2007647 | 0 | 0 | 0 | 0 | 0 | 22.212 | 0 |
| SMINT2007729 | 0 | 40.363 | 0 | 0 | 0 | 0 | 0 |
| SMINT2007792 | 0 | 0 | 0 | 5.932 | 0 | 0 | 0 |
| SMINT2008133 | 0 | 0 | 0 | 16.746 | 0 | 0 | 0 |
| SMINT2008672 | 0 | 0 | 0 | 19.291 | 0 | 0 | 0 |
| SMINT2008921 | 0 | 0 | 0 | 1.575 | 0 | 0 | 0 |
| SMINT2008960 | 0 | 4.802 | 0 | 0.971 | 0 | 3.447 | 0 |
| SMINT2009233 | 0 | 0 | 0 | 16.746 | 0 | 0 | 0 |
| SMINT2010084 | 0 | 0 | 0 | 4.878 | 0 | 0 | 0 |
| SMINT2010639 | 0 | 0 | 0 | 9.613 | 0 | 0 | 0 |
| SMINT2011656 | 0 | 0 | 0 | 16.746 | 0 | 0 | 0 |
| SMINT2012195 | 0 | 0 | 0 | 16.746 | 0 | 0 | 0 |
| SMINT2012291 | 0 | 0 | 0 | 6.546 | 0 | 0 | 0 |
| SMINT2012501 | 0 | 55.814 | 0 | 0 | 0 | 8.012 | 0 |
| SMINT2013032 | 0 | 0 | 0 | 0 | 0 | 12.051 | 0 |
| SMINT2013181 | 0 | 0 | 0 | 0 | 0 | 22.212 | 0 |
| SMINT2017884 | 0 | 0 | 0 | 16.746 | 0 | 0 | 0 |
| SPLEN1000106 | 0 | 0 | 0 | 4.956 | 0 | 0 | 0 |
| SPLEN1000116 | 0 | 3.474 | 0 | 1.054 | 0 | 0 | 0 |
| SPLEN1000165 | 0 | 0 | 0 | 0 | 0 | 10.057 | 0 |
| SPLEN1000166 | 0 | 0 | 0 | 0.802 | 0 | 2.278 | 0 |
| SPLEN2000098 | 0 | 0 | 0 | 17.098 | 0 | 36.410 | 0 |
| SPLEN2000255 | 0 | 0 | 0 | 2.180 | 5.549 | 0 | 0 |
| SPLEN2000267 | 0 | 0 | 0 | 0 | 0 | 5.882 | 0 |
| SPLEN2000341 | 0 | 0 | 0 | 0 | 0 | 13.644 | 0 |
| SPLEN2000348 | 0 | 0 | 0 | 0 | 0 | 2.819 | 0 |
| SPLEN2000443 | 0 | 0 | 0 | 0 | 0 | 22.258 | 0 |
| SPLEN2000505 | 0 | 0 | 0 | 0.598 | 0 | 4.671 | 0 |
| SPLEN2000698 | 0 | 0 | 0 | 0 | 0 | 2.137 | 0 |
| SPLEN2000874 | 15.278 | 0 | 0 | 0 | 1.761 | 0 | 0 |
| SPLEN2001141 | 0 | 0 | 0 | 0.670 | 0 | 0 | 0 |
| SPLEN2001157 | 0 | 0 | 0 | 1.952 | 0 | 2.771 | 0 |
| SPLEN2001599 | 0 | 0 | 0 | 0 | 0 | 7.190 | 0 |
| SPLEN2001761 | 0 | 0 | 0 | 0 | 0 | 21.443 | 0 |
| SPLEN2002007 | 0 | 0 | 0 | 0 | 0 | 19.229 | 0 |
| SPLEN2002335 | 0 | 7.107 | 7.809 | 1.437 | 0 | 3.061 | 0 |
| SPLEN2002463 | 0 | 0 | 0 | 16.124 | 0 | 0 | 0 |
| SPLEN2002467 | 0 | 0 | 0 | 0 | 0 | 6.565 | 0 |
| SPLEN2002493 | 0 | 0 | 0 | 22.112 | 0 | 5.232 | 0 |
| SPLEN2004343 | 0 | 0 | 0 | 12.769 | 0 | 0 | 0 |
| SPLEN2004555 | 0 | 0 | 0 | 0 | 3.846 | 1.072 | 0 |
| SPLEN2004662 | 0 | 0 | 0 | 13.369 | 0 | 0 | 0 |
| SPLEN2004773 | 0 | 0 | 0 | 0 | 7.715 | 3.585 | 0 |
| SPLEN2004844 | 0 | 80.138 | 0 | 0 | 0 | 0 | 0 |
| SPLEN2005429 | 0 | 0 | 0 | 5.201 | 0 | 0 | 0 |
| SPLEN2005783 | 0 | 0 | 0 | 9.254 | 0 | 0 | 0 |
| SPLEN2005806 | 0 | 0 | 0 | 0 | 0 | 0.616 | 0 |
| SPLEN2006133 | 0 | 6.658 | 0 | 1.347 | 3.428 | 0.956 | 0 |
| SPLEN2006143 | 0 | 0 | 0 | 1.588 | 0 | 0 | 0 |
| SPLEN2006305 | 0 | 0 | 4.871 | 1.345 | 0 | 0.636 | 0 |
| SPLEN2006374 | 0 | 0 | 0 | 7.552 | 0 | 1.532 | 0 |
| SPLEN2006671 | 0 | 0 | 0 | 0 | 67.502 | 0 | 0 |
| SPLEN2007498 | 0 | 0 | 0 | 1.857 | 9.456 | 2.637 | 0 |
| SPLEN2007739 | 0 | 0 | 0 | 4.310 | 0 | 0 | 0 |
| SPLEN2008496 | 0 | 0 | 59.707 | 0 | 0 | 0 | 0 |
| SPLEN2009513 | 0 | 0 | 0 | 4.400 | 0 | 0 | 0 |
| SPLEN2010015 | 0 | 0 | 0 | 11.034 | 0 | 0 | 0 |
| SPLEN2010195 | 0 | 0 | 0 | 0.826 | 1.402 | 1.564 | 0 |
| SPLEN2010847 | 0 | 0 | 0 | 11.707 | 59.599 | 0 | 0 |
| SPLEN2010912 | 0 | 0 | 0 | 26.371 | 0 | 0 | 0 |
| SPLEN2010986 | 0 | 0 | 0 | 14.293 | 0 | 0 | 0 |
| SPLEN2011419 | 0 | 0 | 18.428 | 0 | 0 | 2.408 | 0 |
| SPLEN2011737 | 0 | 0 | 0 | 5.548 | 0 | 0 | 0 |
| SPLEN2011758 | 0 | 0 | 40.503 | 0 | 0 | 0 | 0 |
| SPLEN2012453 | 0 | 0 | 0 | 28.977 | 0 | 0 | 0 |
| SPLEN2012571 | 0 | 0 | 0 | 0 | 0 | 3.203 | 0 |
| SPLEN2012624 | 0 | 0 | 0 | 7.178 | 0 | 0 | 0 |
| SPLEN2013195 | 0 | 0 | 0 | 28.977 | 0 | 0 | 0 |
| SPLEN2013670 | 0 | 0 | 0 | 16.644 | 0 | 0 | 0 |
| SPLEN2014080 | 0 | 0 | 0 | 3.363 | 0 | 0 | 0 |
| SPLEN2014119 | 0 | 0 | 0 | 11.845 | 0 | 0 | 0 |
| SPLEN2014911 | 0 | 0 | 0 | 0.749 | 0 | 0 | 0 |
| SPLEN2015310 | 0 | 0 | 0 | 0.814 | 0 | 1.733 | 0 |
| SPLEN2015788 | 0 | 0 | 0 | 3.436 | 0 | 0 | 0 |
| SPLEN2015890 | 0 | 0 | 0 | 28.977 | 0 | 0 | 0 |
| SPLEN2016139 | 0 | 8.993 | 0 | 0.909 | 0 | 0 | 0 |
| SPLEN2016268 | 0 | 3.032 | 0 | 0.307 | 0 | 0.435 | 0 |
| SPLEN2016773 | 0 | 0 | 0 | 28.977 | 0 | 0 | 0 |
| SPLEN2016972 | 0 | 0 | 0 | 2.878 | 0 | 0 | 0 |
| SPLEN2017121 | 0 | 0 | 0 | 0 | 0 | 15.624 | 0 |
| SPLEN2017318 | 0 | 0 | 0 | 3.601 | 0 | 0 | 0 |
| SPLEN2017351 | 0 | 0 | 0 | 2.135 | 0 | 0 | 0 |
| SPLEN2017592 | 0 | 0 | 0 | 28.977 | 0 | 0 | 0 |
| SPLEN2017781 | 0 | 0 | 0 | 28.977 | 0 | 0 | 0 |
| SPLEN2017918 | 0 | 6.399 | 7.031 | 0 | 0 | 0 | 0 |
| SPLEN2019169 | 0 | 0 | 0 | 24.109 | 0 | 0 | 0 |
| SPLEN2019257 | 0 | 0 | 0 | 28.977 | 0 | 0 | 0 |
| SPLEN2019379 | 0 | 80.138 | 0 | 0 | 0 | 0 | 0 |
| SPLEN2019405 | 0 | 10.051 | 0 | 1.016 | 0 | 7.215 | 0 |
| SPLEN2019956 | 0 | 0 | 0 | 21.618 | 0 | 0 | 0 |
| SPLEN2020007 | 0 | 0 | 0 | 0 | 67.502 | 0 | 0 |
| SPLEN2020417 | 0 | 0 | 0 | 9.907 | 0 | 0 | 0 |
| SPLEN2024489 | 0 | 0 | 0 | 7.755 | 0 | 0 | 0 |
| SPLEN2025039 | 0 | 0 | 0 | 28.977 | 0 | 0 | 0 |
| SPLEN2027113 | 0 | 0 | 0 | 4.646 | 0 | 0 | 0 |
| SPLEN2028466 | 0 | 0 | 3.190 | 1.174 | 0 | 5.002 | 0 |
| SPLEN2029912 | 0 | 0 | 0 | 0 | 0 | 1.646 | 0 |
| SPLEN2030763 | 0 | 0 | 0 | 7.475 | 0 | 0 | 0 |
| SPLEN2031424 | 0 | 0 | 0 | 5.405 | 0 | 0 | 0 |
| SPLEN2031547 | 0 | 0 | 0 | 5.587 | 5.689 | 4.759 | 0 |
| SPLEN2034448 | 0 | 0 | 0 | 0 | 0 | 5.224 | 0 |
| SPLEN2036932 | 0 | 0 | 0 | 2.081 | 0 | 0 | 0 |
| SPLEN2037194 | 0 | 0 | 0 | 28.977 | 0 | 0 | 0 |
| SPLEN2038345 | 0 | 0 | 0 | 0 | 0 | 29.627 | 0 |
| SPLEN2039379 | 0 | 0 | 0 | 10.278 | 0 | 11.672 | 0 |
| SPLEN2040237 | 0 | 0 | 0 | 28.977 | 0 | 0 | 0 |
| SPLEN2042535 | 0 | 0 | 0 | 0 | 0 | 7.174 | 0 |
| STOMA2000289 | 0 | 0 | 0 | 5.801 | 9.844 | 5.490 | 0 |
| STOMA2000395 | 0 | 14.983 | 0 | 3.030 | 0 | 4.302 | 0 |
| STOMA2000396 | 0 | 0 | 0 | 3.658 | 0 | 0 | 0 |
| STOMA2000482 | 0 | 0 | 0 | 2.736 | 0 | 3.884 | 0 |
| STOMA2002052 | 0 | 0 | 0 | 3.948 | 0 | 5.604 | 0 |
| STOMA2002166 | 0 | 0 | 0 | 0.415 | 0 | 0.589 | 0 |
| STOMA2009289 | 0 | 0 | 0 | 4.072 | 0 | 0 | 0 |
| SYNOV1000124 | 0 | 0 | 0 | 1.184 | 4.020 | 11.209 | 0 |
| SYNOV2000820 | 0 | 0 | 0 | 24.760 | 0 | 0 | 0 |
| SYNOV2001033 | 0 | 0 | 0 | 0.426 | 2.166 | 0.604 | 0 |
| SYNOV2001144 | 0 | 0 | 0 | 0 | 0 | 1.425 | 0 |
| SYNOV2001239 | 0 | 2.202 | 0 | 0.445 | 3.401 | 0.316 | 0 |
| SYNOV2001262 | 0 | 14.644 | 0 | 1.481 | 0 | 2.102 | 0 |
| SYNOV2009172 | 0 | 0 | 0 | 1.108 | 1.880 | 0 | 0 |
| SYNOV2021953 | 0 | 0 | 0 | 6.793 | 0 | 0 | 0 |
| SYNOV4001153 | 0 | 0 | 0 | 14.816 | 0 | 2.337 | 0 |
| SYNOV4007671 | 0 | 0 | 0 | 0.520 | 2.649 | 0.739 | 0 |
| SYNOV4008245 | 0 | 0 | 0 | 6.557 | 0 | 0 | 0 |
| SYNOV4009575 | 0 | 0 | 0 | 0 | 0 | 4.828 | 0 |
| TBAES2001296 | 0 | 0 | 0 | 0 | 0 | 11.352 | 0 |
| TBAES2009387 | 0 | 0 | 0 | 1.518 | 0 | 0 | 0 |
| TCERX2000431 | 0 | 0 | 0 | 0 | 0 | 2.117 | 0 |
| TCERX2002644 | 0 | 0 | 0 | 0 | 0 | 3.457 | 0 |
| TCOLN1000180 | 0 | 17.184 | 0 | 1.738 | 8.846 | 4.934 | 0 |
| TESOP2000312 | 0 | 33.903 | 0 | 0 | 0 | 0 | 0 |
| TESOP2002273 | 0 | 0 | 0 | 2.881 | 14.666 | 0 | 0 |
| TESOP2002451 | 0 | 0 | 0 | 3.372 | 0 | 0 | 0 |
| TESOP2002950 | 0 | 0 | 0 | 9.399 | 0 | 0 | 0 |
| TESTI1000023 | 0 | 0 | 0 | 0.595 | 0 | 6.760 | 0 |
| TESTI1000070 | 0 | 0 | 0 | 0 | 0 | 3.638 | 0 |
| TESTI1000096 | 0 | 0 | 0 | 12.416 | 0 | 0 | 0 |
| TESTI1000168 | 0 | 0 | 0 | 0 | 0 | 6.315 | 0 |
| TESTI1000330 | 0 | 0 | 0 | 52.057 | 0 | 0 | 0 |
| TESTI2000100 | 0 | 0 | 0 | 0 | 0 | 39.041 | 0 |
| TESTI2000184 | 0 | 0 | 0 | 4.012 | 0 | 1.139 | 0 |
| TESTI2000272 | 0 | 0 | 0 | 6.872 | 0 | 0 | 0 |
| TESTI2000356 | 0 | 0 | 0 | 12.446 | 0 | 0 | 0 |
| TESTI2000598 | 0 | 0 | 0 | 3.565 | 0 | 5.061 | 0 |
| TESTI2000627 | 0 | 0 | 0 | 14.955 | 0 | 0 | 0 |
| TESTI2000909 | 0 | 91.480 | 0 | 0 | 0 | 0 | 0 |
| TESTI2000970 | 0 | 0 | 0 | 0 | 17.565 | 4.898 | 0 |
| TESTI2001099 | 0 | 0 | 3.779 | 0.696 | 0 | 0.987 | 0 |
| TESTI2001153 | 0 | 0 | 0 | 0 | 0 | 0.802 | 0 |
| TESTI2001269 | 0 | 0 | 0 | 1.162 | 1.972 | 1.100 | 0 |
| TESTI2001511 | 0 | 0 | 0 | 0 | 0 | 1.763 | 0 |
| TESTI2001613 | 0 | 0 | 0 | 9.696 | 0 | 0 | 0 |
| TESTI2001829 | 0 | 0 | 0 | 0.603 | 0 | 0 | 0 |
| TESTI2001862 | 0 | 0 | 0 | 3.753 | 0 | 0 | 0 |
| TESTI2001985 | 0 | 0 | 0 | 4.166 | 0 | 0 | 0 |
| TESTI2002365 | 0 | 0 | 0 | 0 | 0 | 1.100 | 0 |
| TESTI2002707 | 0 | 0 | 0 | 3.528 | 5.987 | 1.669 | 0 |
| TESTI2003005 | 0 | 0 | 0 | 2.156 | 0 | 0 | 0 |
| TESTI2003071 | 0 | 0 | 0 | 2.476 | 0 | 0 | 0 |
| TESTI2003127 | 0 | 0 | 0 | 0 | 0 | 10.759 | 0 |
| TESTI2003141 | 0 | 0 | 0 | 0 | 0 | 11.370 | 0 |
| TESTI2003280 | 0 | 0 | 0 | 8.734 | 29.644 | 0 | 0 |
| TESTI2003325 | 0 | 0 | 0 | 6.008 | 0 | 0 | 0 |
| TESTI2003533 | 0 | 0 | 0 | 0.615 | 0 | 0.874 | 0 |
| TESTI2003541 | 0 | 0 | 0 | 0 | 0 | 29.428 | 0 |
| TESTI2003625 | 0 | 0 | 0 | 0 | 0 | 7.712 | 0 |
| TESTI2004031 | 0 | 2.814 | 0 | 0.285 | 0 | 0 | 0 |
| TESTI2004080 | 0 | 34.254 | 0 | 0 | 0 | 0 | 0 |
| TESTI2004122 | 0 | 0 | 0 | 2.167 | 0 | 0 | 0 |
| TESTI2004153 | 0 | 0 | 0 | 6.158 | 0 | 0 | 0 |
| TESTI2004295 | 0 | 0 | 0 | 0 | 0 | 8.251 | 0 |
| TESTI2004737 | 0 | 0 | 13.660 | 2.514 | 0 | 0 | 0 |
| TESTI2004929 | 0 | 0 | 0 | 0 | 0 | 2.861 | 0 |
| TESTI2005072 | 0 | 0 | 0 | 3.282 | 0 | 0 | 0 |
| TESTI2005408 | 0 | 0 | 0 | 0 | 0 | 1.785 | 0 |
| TESTI2005742 | 0 | 0 | 0 | 0 | 0 | 20.548 | 0 |
| TESTI2005743 | 0 | 0 | 0 | 4.677 | 0 | 0 | 0 |
| TESTI2005759 | 58.629 | 0 | 0 | 0 | 0 | 0 | 0 |
| TESTI2005767 | 0 | 0 | 0 | 1.941 | 0 | 0 | 0 |
| TESTI2005784 | 0 | 0 | 0 | 0 | 0 | 2.997 | 0 |
| TESTI2005788 | 0 | 0 | 0 | 0 | 0 | 4.660 | 0 |
| TESTI2005908 | 0 | 0 | 0 | 1.118 | 17.069 | 1.587 | 0 |
| TESTI2005981 | 0 | 0 | 0 | 22.105 | 0 | 0 | 0 |
| TESTI2006051 | 0 | 0 | 0 | 0.301 | 0 | 0.427 | 0 |
| TESTI2006097 | 0 | 0 | 0 | 7.478 | 0 | 0 | 0 |
| TESTI2006111 | 0 | 0 | 0 | 14.633 | 0 | 0 | 0 |
| TESTI2006120 | 0 | 0 | 0 | 0 | 0 | 4.890 | 0 |
| TESTI2006210 | 0 | 0 | 66.287 | 0 | 0 | 0 | 0 |
| TESTI2006341 | 0 | 0 | 0 | 14.508 | 0 | 0 | 0 |
| TESTI2006383 | 0 | 0 | 0 | 0 | 0 | 4.084 | 0 |
| TESTI2006453 | 0 | 0 | 0 | 0.597 | 0 | 4.240 | 0 |
| TESTI2006588 | 0 | 0 | 0 | 2.305 | 3.912 | 0.545 | 0 |
| TESTI2006615 | 0 | 0 | 0 | 3.276 | 0 | 0 | 0 |
| TESTI2006748 | 0 | 0 | 0 | 4.883 | 0 | 0 | 0 |
| TESTI2006774 | 0 | 0 | 0 | 52.057 | 0 | 0 | 0 |
| TESTI2007163 | 0 | 0 | 0 | 52.057 | 0 | 0 | 0 |
| TESTI2007211 | 0 | 23.183 | 0 | 2.344 | 11.934 | 0 | 0 |
| TESTI2007311 | 0 | 0 | 0 | 2.731 | 0 | 3.876 | 0 |
| TESTI2007407 | 0 | 0 | 0 | 6.536 | 11.091 | 0 | 0 |
| TESTI2007998 | 0 | 0 | 0 | 0 | 0 | 1.593 | 0 |
| TESTI2008240 | 0 | 1.755 | 0 | 5.502 | 0 | 3.779 | 0 |
| TESTI2008847 | 0 | 0 | 0 | 0 | 0 | 143.525 | 0 |
| TESTI2009239 | 0 | 0 | 0 | 52.057 | 0 | 0 | 0 |
| TESTI2009388 | 0 | 0 | 0 | 14.231 | 0 | 0 | 0 |
| TESTI2009402 | 0 | 0 | 0 | 0 | 0 | 9.237 | 0 |
| TESTI2009474 | 0 | 0 | 0 | 0 | 0 | 2.036 | 0 |
| TESTI2009520 | 0 | 0 | 0 | 0.532 | 2.710 | 0.756 | 0 |
| TESTI2009683 | 0 | 0 | 0 | 0 | 0 | 60.652 | 0 |
| TESTI2009785 | 0 | 0 | 0 | 1.018 | 0 | 1.446 | 0 |
| TESTI2009987 | 0 | 0 | 0 | 10.862 | 0 | 10.280 | 0 |
| TESTI2010513 | 0 | 0 | 0 | 12.510 | 12.839 | 1.979 | 0 |
| TESTI2010617 | 0 | 0 | 0 | 0 | 0 | 7.017 | 0 |
| TESTI2011549 | 0 | 0 | 0 | 0 | 0 | 60.652 | 0 |
| TESTI2011605 | 0 | 50.596 | 0 | 0 | 0 | 0 | 0 |
| TESTI2012104 | 0 | 0 | 0 | 6.910 | 0 | 0 | 0 |
| TESTI2012289 | 0 | 0 | 0 | 6.126 | 0 | 0 | 0 |
| TESTI2012511 | 0 | 0 | 0 | 0 | 0 | 4.326 | 0 |
| TESTI2012778 | 5.792 | 0 | 4.275 | 0 | 0 | 0.186 | 0 |
| TESTI2013231 | 0 | 0 | 0 | 1.412 | 0 | 0 | 0 |
| TESTI2013295 | 0 | 0 | 0 | 1.890 | 0 | 0 | 0 |
| TESTI2014318 | 0 | 0 | 0 | 0 | 0 | 28.966 | 0 |
| TESTI2014838 | 0 | 0 | 0 | 0 | 0 | 1.335 | 0 |
| TESTI2015092 | 0 | 0 | 0 | 0 | 11.971 | 0 | 0 |
| TESTI2015213 | 0 | 0 | 3.958 | 0.729 | 0 | 0.517 | 0 |
| TESTI2015246 | 0 | 0 | 0 | 8.403 | 0 | 0 | 0 |
| TESTI2015626 | 0 | 4.617 | 2.536 | 0.467 | 0 | 0.331 | 0 |
| TESTI2015962 | 0 | 0 | 0 | 14.078 | 0 | 15.988 | 0 |
| TESTI2016583 | 0 | 0 | 0 | 12.766 | 0 | 0 | 0 |
| TESTI2016701 | 0 | 0 | 0 | 52.057 | 0 | 0 | 0 |
| TESTI2017035 | 0 | 0 | 0 | 29.776 | 0 | 0 | 0 |
| TESTI2017107 | 0 | 0 | 0 | 0 | 0 | 60.652 | 0 |
| TESTI2017954 | 0 | 0 | 0 | 0.470 | 0 | 0 | 0 |
| TESTI2018014 | 0 | 0 | 0 | 10.943 | 0 | 0 | 0 |
| TESTI2018462 | 0 | 0 | 0 | 0 | 0 | 5.695 | 0 |
| TESTI2018867 | 0 | 0 | 0 | 4.042 | 0 | 0 | 0 |
| TESTI2018997 | 0 | 0 | 0 | 0 | 0 | 2.099 | 0 |
| TESTI2019760 | 0 | 0 | 0 | 32.302 | 0 | 0 | 0 |
| TESTI2020840 | 0 | 0 | 0 | 11.599 | 0 | 0 | 0 |
| TESTI2021038 | 0 | 0 | 0 | 30.031 | 0 | 0 | 0 |
| TESTI2021124 | 0 | 0 | 0 | 0 | 0 | 5.759 | 0 |
| TESTI2021425 | 0 | 0 | 0 | 1.222 | 0 | 0 | 0 |
| TESTI2021531 | 0 | 0 | 0 | 11.875 | 0 | 16.859 | 0 |
| TESTI2021654 | 0 | 0 | 0 | 0 | 0 | 9.847 | 0 |
| TESTI2021851 | 0 | 0 | 0 | 0 | 0 | 19.064 | 0 |
| TESTI2023414 | 0 | 0 | 0 | 2.889 | 0 | 0 | 0 |
| TESTI2025422 | 0 | 0 | 0 | 1.762 | 0 | 0 | 0 |
| TESTI2025846 | 0 | 0 | 0 | 43.347 | 0 | 0 | 0 |
| TESTI2026116 | 0 | 0 | 0 | 10.586 | 0 | 0 | 0 |
| TESTI2026525 | 0 | 0 | 0 | 3.088 | 0 | 0 | 0 |
| TESTI2026647 | 0 | 0 | 0 | 0 | 0 | 23.811 | 0 |
| TESTI2026936 | 0 | 0 | 0 | 0 | 0 | 60.652 | 0 |
| TESTI2028296 | 0 | 0 | 0 | 1.750 | 17.820 | 0 | 0 |
| TESTI2028659 | 0 | 0 | 0 | 21.395 | 0 | 0 | 0 |
| TESTI2029227 | 0 | 0 | 0 | 0 | 0 | 11.962 | 0 |
| TESTI2033441 | 0 | 0 | 8.462 | 0 | 0 | 0 | 0 |
| TESTI2034916 | 0 | 0 | 0 | 10.791 | 0 | 0 | 0 |
| TESTI2034940 | 0 | 0 | 0 | 2.589 | 1.883 | 0 | 0 |
| TESTI2035003 | 0 | 0 | 0 | 52.057 | 0 | 0 | 0 |
| TESTI2035236 | 0 | 0 | 0 | 0 | 0 | 25.974 | 0 |
| TESTI2036650 | 0 | 0 | 0 | 0 | 0 | 60.652 | 0 |
| TESTI2036969 | 0 | 0 | 0 | 2.550 | 0 | 0 | 0 |
| TESTI2039177 | 0 | 0 | 0 | 5.883 | 0 | 0 | 0 |
| TESTI2043275 | 0 | 0 | 0 | 0 | 31.972 | 0 | 0 |
| TESTI2045155 | 0 | 0 | 0 | 0 | 0 | 11.606 | 0 |
| TESTI2046456 | 0 | 0 | 0 | 0.657 | 1.673 | 0.466 | 0 |
| TESTI2053723 | 0 | 0 | 0 | 6.426 | 0 | 4.561 | 0 |
| TESTI4000014 | 0 | 0 | 0 | 2.028 | 0 | 1.799 | 0 |
| TESTI4000349 | 0 | 0 | 0 | 36.899 | 0 | 0 | 0 |
| TESTI4000434 | 0 | 0 | 0 | 9.280 | 0 | 0 | 0 |
| TESTI4001467 | 0 | 38.859 | 0 | 0 | 0 | 0 | 0 |
| TESTI4001561 | 0 | 0 | 0 | 28.578 | 0 | 0 | 0 |
| TESTI4002072 | 0 | 0 | 0 | 11.850 | 0 | 3.364 | 0 |
| TESTI4002195 | 0 | 0 | 51.594 | 0 | 0 | 0 | 0 |
| TESTI4002202 | 0 | 0 | 0 | 7.059 | 0 | 0 | 0 |
| TESTI4002799 | 0 | 0 | 0 | 0.627 | 0 | 0 | 0 |
| TESTI4002988 | 0 | 0 | 0 | 15.392 | 0 | 21.851 | 0 |
| TESTI4003150 | 0 | 0 | 0 | 10.540 | 0 | 0 | 0 |
| TESTI4003602 | 0 | 0 | 0 | 18.431 | 0 | 0 | 0 |
| TESTI4003703 | 0 | 0 | 0 | 6.281 | 0 | 4.458 | 0 |
| TESTI4004626 | 0 | 0 | 0 | 11.685 | 0 | 0 | 0 |
| TESTI4005133 | 0 | 0 | 0 | 16.927 | 0 | 12.015 | 0 |
| TESTI4005399 | 0 | 0 | 0 | 11.068 | 0 | 15.713 | 0 |
| TESTI4007369 | 0 | 91.480 | 0 | 0 | 0 | 0 | 0 |
| TESTI4009454 | 0 | 0 | 0 | 0 | 29.045 | 8.099 | 0 |
| TESTI4010851 | 0 | 0 | 0 | 2.716 | 0 | 2.203 | 0 |
| TESTI4013441 | 4.916 | 1.101 | 1.210 | 0.223 | 0 | 0.316 | 0 |
| TESTI4013474 | 0 | 0 | 0 | 0 | 3.733 | 0 | 0 |
| TESTI4013675 | 0 | 0 | 0 | 14.015 | 0 | 0 | 0 |
| TESTI4013817 | 0 | 0 | 0 | 0 | 0 | 27.369 | 0 |
| TESTI4013894 | 0 | 0 | 0 | 19.881 | 0 | 14.112 | 0 |
| TESTI4014159 | 0 | 0 | 0 | 16.638 | 0 | 11.810 | 0 |
| TESTI4014306 | 0 | 0 | 0 | 52.057 | 0 | 0 | 0 |
| TESTI4014553 | 0 | 0 | 0 | 68.470 | 0 | 0 | 0 |
| TESTI4014694 | 0 | 0 | 0 | 0.791 | 0 | 0 | 0 |
| TESTI4017984 | 0 | 0 | 0 | 35.187 | 0 | 0 | 0 |
| TESTI4022158 | 0 | 0 | 0 | 9.473 | 0 | 0 | 0 |
| TESTI4024740 | 0 | 0 | 0 | 0 | 0 | 18.126 | 0 |
| TESTI4026390 | 0 | 0 | 0 | 52.057 | 0 | 0 | 0 |
| TESTI4032913 | 0 | 0 | 0 | 5.753 | 0 | 8.167 | 0 |
| TESTI4037156 | 0 | 0 | 12.804 | 0.982 | 0 | 1.394 | 0 |
| TESTI4037188 | 0 | 0 | 0 | 0 | 0 | 60.652 | 0 |
| TESTI4038099 | 0 | 0 | 0 | 15.656 | 0 | 0 | 0 |
| TESTI4038284 | 0 | 0 | 0 | 7.843 | 0 | 0 | 0 |
| TESTI4043140 | 0 | 0 | 0 | 0 | 10.019 | 0 | 0 |
| TESTI4043223 | 0 | 0 | 0 | 19.643 | 0 | 27.886 | 0 |
| TESTI4044704 | 0 | 0 | 0 | 7.235 | 0 | 0 | 0 |
| TESTI4046073 | 0 | 0 | 0 | 0 | 0 | 19.130 | 0 |
| TESTI4046819 | 0 | 0 | 0 | 14.013 | 0 | 0 | 0 |
| THYMU1000002 | 0 | 0 | 0 | 0 | 0 | 0.596 | 0 |
| THYMU1000016 | 0 | 2.712 | 0 | 0 | 0 | 0 | 0 |
| THYMU1000032 | 0 | 5.479 | 0 | 0 | 0 | 0 | 0 |
| THYMU1000041 | 0 | 0 | 0 | 3.375 | 0 | 2.396 | 0 |
| THYMU1000083 | 0 | 0 | 0 | 0 | 0 | 3.021 | 0 |
| THYMU1000103 | 0 | 0 | 0 | 0 | 0 | 7.106 | 0 |
| THYMU1000109 | 0 | 0 | 0 | 1.676 | 8.532 | 0 | 0 |
| THYMU1000144 | 0 | 0 | 0 | 6.426 | 0 | 3.909 | 0 |
| THYMU1000198 | 0 | 0 | 0 | 0 | 0 | 54.718 | 0 |
| THYMU1000331 | 0 | 0 | 0 | 32.302 | 0 | 0 | 0 |
| THYMU1000359 | 0 | 0 | 0 | 0 | 0 | 0.540 | 0 |
| THYMU1000374 | 0 | 0 | 0 | 2.126 | 0 | 0 | 0 |
| THYMU2000162 | 0 | 0 | 0 | 45.981 | 0 | 0 | 0 |
| THYMU2000382 | 0 | 4.254 | 0 | 0.430 | 0 | 0 | 0 |
| THYMU2000436 | 0 | 6.467 | 0 | 1.962 | 0 | 0 | 0 |
| THYMU2000489 | 0 | 0 | 0 | 0 | 0 | 54.718 | 0 |
| THYMU2000684 | 0 | 0 | 0 | 0 | 0 | 4.195 | 0 |
| THYMU2000946 | 0 | 10.155 | 0 | 1.027 | 0 | 1.458 | 0 |
| THYMU2001007 | 0 | 0 | 0 | 0 | 0 | 2.733 | 0 |
| THYMU2001839 | 0 | 0 | 0 | 0 | 0 | 15.764 | 0 |
| THYMU2002162 | 0 | 0 | 0 | 0 | 0 | 10.985 | 0 |
| THYMU2002583 | 0 | 0 | 3.997 | 2.575 | 0 | 0.522 | 0 |
| THYMU2002841 | 0 | 20.370 | 0 | 0 | 0 | 2.924 | 0 |
| THYMU2002983 | 0 | 21.005 | 0 | 0 | 0 | 0 | 0 |
| THYMU2003012 | 0 | 0 | 0 | 0.622 | 3.167 | 0.883 | 0 |
| THYMU2003046 | 0 | 0 | 0 | 1.364 | 0 | 0 | 0 |
| THYMU2003133 | 0 | 24.353 | 0 | 0 | 0 | 0 | 0 |
| THYMU2003419 | 0 | 0 | 0 | 2.495 | 0 | 2.024 | 0 |
| THYMU2003446 | 0 | 0 | 0 | 3.386 | 0 | 4.806 | 0 |
| THYMU2003542 | 0 | 15.168 | 0 | 0 | 0 | 0 | 0 |
| THYMU2003891 | 0 | 1.024 | 0 | 0.104 | 0 | 0 | 0 |
| THYMU2004152 | 0 | 6.969 | 0 | 0 | 3.588 | 0 | 0 |
| THYMU2004284 | 0 | 0 | 0 | 4.768 | 0 | 0 | 0 |
| THYMU2004344 | 0 | 0 | 0 | 0 | 0 | 2.574 | 0 |
| THYMU2004410 | 0 | 0 | 0 | 5.440 | 0 | 0 | 0 |
| THYMU2004508 | 0 | 0 | 0 | 45.981 | 0 | 0 | 0 |
| THYMU2004733 | 0 | 0 | 0 | 3.975 | 0 | 0 | 0 |
| THYMU2005574 | 0 | 0 | 0 | 45.981 | 0 | 0 | 0 |
| THYMU2005807 | 0 | 0 | 0 | 45.981 | 0 | 0 | 0 |
| THYMU2006505 | 0 | 9.582 | 0 | 1.938 | 0 | 4.126 | 0 |
| THYMU2006813 | 0 | 22.213 | 0 | 4.492 | 5.718 | 0 | 0 |
| THYMU2007036 | 0 | 0 | 0 | 0 | 0 | 7.780 | 0 |
| THYMU2007065 | 0 | 0 | 0 | 2.878 | 0 | 4.086 | 0 |
| THYMU2007415 | 0 | 16.985 | 0 | 1.718 | 0 | 0 | 0 |
| THYMU2007467 | 0 | 0 | 0 | 5.734 | 0 | 0 | 0 |
| THYMU2007532 | 0 | 0 | 0 | 0 | 56.448 | 0 | 0 |
| THYMU2007694 | 0 | 0 | 0 | 0 | 0 | 20.890 | 0 |
| THYMU2007729 | 0 | 0 | 0 | 3.469 | 0 | 0 | 0 |
| THYMU2007886 | 0 | 0 | 0 | 0 | 5.003 | 0 | 0 |
| THYMU2008036 | 0 | 0 | 0 | 0 | 0 | 54.718 | 0 |
| THYMU2008321 | 0 | 0 | 0 | 2.052 | 0 | 5.826 | 0 |
| THYMU2009592 | 0 | 0 | 0 | 19.608 | 0 | 0 | 0 |
| THYMU2009658 | 0 | 2.459 | 0 | 0.746 | 0 | 0.353 | 0 |
| THYMU2010041 | 0 | 0 | 0 | 0 | 0 | 18.787 | 0 |
| THYMU2010448 | 0 | 0 | 0 | 0 | 0 | 5.335 | 0 |
| THYMU2010779 | 0 | 0 | 0 | 29.854 | 0 | 0 | 0 |
| THYMU2010816 | 0 | 0 | 0 | 0 | 0 | 26.271 | 0 |
| THYMU2011063 | 0 | 0 | 81.157 | 0 | 0 | 5.302 | 0 |
| THYMU2011482 | 0 | 0 | 0 | 0 | 22.183 | 0 | 0 |
| THYMU2011939 | 0 | 0 | 0 | 45.981 | 0 | 0 | 0 |
| THYMU2012024 | 0 | 0 | 0 | 2.115 | 0 | 0 | 0 |
| THYMU2012690 | 0 | 0 | 0 | 0 | 0 | 3.915 | 0 |
| THYMU2012826 | 0 | 0 | 0 | 0 | 0 | 22.315 | 0 |
| THYMU2013136 | 0 | 0 | 0 | 0.581 | 0 | 0.824 | 0 |
| THYMU2013916 | 0 | 0 | 0 | 1.574 | 0 | 2.235 | 0 |
| THYMU2014323 | 0 | 0 | 0 | 0 | 0 | 54.718 | 0 |
| THYMU2014353 | 0 | 0 | 0 | 0 | 0 | 37.663 | 0 |
| THYMU2015441 | 0 | 0 | 0 | 0 | 81.250 | 0 | 0 |
| THYMU2016523 | 0 | 0 | 0 | 23.416 | 0 | 0 | 0 |
| THYMU2016840 | 0 | 0 | 0 | 0 | 81.250 | 0 | 0 |
| THYMU2017601 | 0 | 0 | 0 | 5.012 | 0 | 0 | 0 |
| THYMU2017878 | 0 | 0 | 0 | 45.981 | 0 | 0 | 0 |
| THYMU2018126 | 0 | 0 | 0 | 0 | 0 | 0.733 | 0 |
| THYMU2019021 | 0 | 0 | 0 | 0 | 0 | 4.897 | 0 |
| THYMU2019364 | 0 | 0 | 0 | 29.854 | 0 | 0 | 0 |
| THYMU2020959 | 0 | 0 | 0 | 7.868 | 0 | 0 | 0 |
| THYMU2023264 | 0 | 0 | 0 | 0 | 0 | 54.718 | 0 |
| THYMU2023967 | 0 | 0 | 0 | 43.363 | 0 | 0 | 0 |
| THYMU2024071 | 0 | 0 | 0 | 0 | 0 | 33.209 | 0 |
| THYMU2025325 | 0 | 0 | 0 | 0 | 0 | 13.123 | 0 |
| THYMU2027125 | 5.611 | 0 | 0 | 2.287 | 2.587 | 0.902 | 0 |
| THYMU2029578 | 0 | 48.262 | 0 | 4.880 | 0 | 0 | 0 |
| THYMU2030264 | 0 | 0 | 0 | 45.981 | 0 | 0 | 0 |
| THYMU2034917 | 0 | 0 | 0 | 13.791 | 0 | 0 | 0 |
| THYMU2035735 | 0 | 0 | 0 | 0.936 | 0 | 0 | 0 |
| THYMU2037226 | 0 | 0 | 0 | 0 | 0 | 17.265 | 0 |
| THYMU2038220 | 0 | 0 | 0 | 0 | 0 | 20.354 | 0 |
| THYMU2039780 | 0 | 0 | 0 | 45.981 | 0 | 0 | 0 |
| THYMU2040427 | 0 | 0 | 0 | 45.981 | 0 | 0 | 0 |
| THYMU2040925 | 0 | 0 | 0 | 45.981 | 0 | 0 | 0 |
| THYMU3000360 | 0 | 0 | 0 | 27.904 | 0 | 0 | 0 |
| THYMU3000420 | 0 | 0 | 0 | 27.904 | 0 | 0 | 0 |
| THYMU3000776 | 0 | 0 | 0 | 22.150 | 0 | 0 | 0 |
| THYMU3001083 | 0 | 0 | 0 | 45.981 | 0 | 0 | 0 |
| THYMU3001379 | 0 | 0 | 0 | 0 | 0 | 25.376 | 0 |
| THYMU3003309 | 0 | 0 | 0 | 20.635 | 0 | 0 | 0 |
| THYMU3004835 | 0 | 0 | 0 | 0 | 0 | 19.793 | 0 |
| THYMU3005629 | 0 | 0 | 0 | 0 | 0 | 15.225 | 0 |
| THYMU3006485 | 0 | 0 | 0 | 45.981 | 0 | 0 | 0 |
| THYMU3007308 | 0 | 0 | 0 | 27.684 | 0 | 0 | 0 |
| THYMU3007423 | 0 | 0 | 0 | 13.957 | 0 | 0 | 0 |
| THYMU3008105 | 0 | 0 | 0 | 0 | 0 | 20.406 | 0 |
| THYMU3008171 | 0 | 0 | 0 | 14.316 | 0 | 0 | 0 |
| THYMU3013386 | 0 | 0 | 0 | 1.063 | 2.705 | 0 | 0 |
| THYMU3015571 | 0 | 73.725 | 0 | 0 | 0 | 0 | 0 |
| THYMU3015759 | 0 | 0 | 8.683 | 0.799 | 0 | 0 | 0 |
| THYMU3019476 | 0 | 0 | 0 | 0 | 0 | 54.718 | 0 |
| THYMU3021586 | 0 | 0 | 0 | 0 | 0 | 4.162 | 0 |
| THYMU3026350 | 0 | 0 | 0 | 0 | 0 | 14.400 | 0 |
| THYMU3032798 | 0 | 0 | 0 | 10.979 | 0 | 1.948 | 0 |
| THYMU3032867 | 0 | 0 | 0 | 9.135 | 0 | 0 | 0 |
| THYMU3033626 | 0 | 0 | 0 | 7.979 | 0 | 0 | 0 |
| THYMU3034671 | 0 | 0 | 0 | 5.078 | 0 | 0 | 0 |
| THYMU3038214 | 0 | 0 | 0 | 27.904 | 0 | 0 | 0 |
| THYMU3041428 | 0 | 0 | 0 | 0 | 0 | 0 | 96.263 |
| THYMU3046350 | 0 | 0 | 0 | 0 | 0 | 6.960 | 0 |
| TKIDN1000001 | 0 | 0 | 0 | 7.899 | 0 | 0 | 0 |
| TKIDN1000164 | 0 | 0 | 0 | 4.388 | 0 | 6.230 | 0 |
| TKIDN1000192 | 0 | 0 | 0 | 0 | 0 | 5.567 | 0 |
| TKIDN2000246 | 0 | 0 | 0 | 0.864 | 12.091 | 2.759 | 0 |
| TKIDN2002318 | 0 | 0 | 0 | 0 | 19.380 | 16.212 | 0 |
| TKIDN2002738 | 0 | 0 | 0 | 0.298 | 0 | 0.845 | 0 |
| TKIDN2003601 | 0 | 0 | 0 | 13.531 | 0 | 0 | 0 |
| TKIDN2005487 | 0 | 0 | 0 | 0.510 | 0 | 0 | 0 |
| TKIDN2006761 | 0 | 0 | 0 | 0 | 0 | 21.396 | 0 |
| TKIDN2007605 | 0 | 0 | 0 | 8.866 | 0 | 0 | 0 |
| TKIDN2007828 | 0 | 0 | 0 | 0 | 0 | 6.723 | 0 |
| TKIDN2008176 | 0 | 0 | 0 | 0 | 0 | 21.396 | 0 |
| TKIDN2009641 | 0 | 0 | 0 | 4.104 | 0 | 5.826 | 0 |
| TKIDN2011289 | 0 | 0 | 0 | 0 | 0 | 6.023 | 0 |
| TKIDN2015071 | 0 | 0 | 0 | 0 | 0 | 13.070 | 0 |
| TKIDN2015285 | 0 | 0 | 0 | 0 | 0 | 10.003 | 0 |
| TKIDN2015423 | 0 | 0 | 0 | 13.555 | 0 | 16.678 | 0 |
| TKIDN2015788 | 0 | 0 | 0 | 0 | 9.505 | 5.301 | 0 |
| TKIDN2016846 | 0 | 0 | 0 | 16.089 | 0 | 0 | 0 |
| TKIDN2018926 | 0 | 0 | 0 | 0 | 0 | 21.396 | 0 |
| TLIVE2002046 | 0 | 0 | 0 | 7.729 | 0 | 10.972 | 0 |
| TLIVE2004320 | 0 | 0 | 0 | 0 | 0 | 12.783 | 0 |
| TLIVE2007736 | 0 | 0 | 0 | 9.358 | 0 | 0 | 0 |
| TLUNG2000179 | 0 | 0 | 0 | 3.316 | 0 | 0 | 0 |
| TRACH1000011 | 0 | 0 | 0 | 5.224 | 0 | 0 | 0 |
| TRACH1000030 | 0 | 0 | 0 | 8.324 | 0 | 1.390 | 0 |
| TRACH1000181 | 0 | 0 | 0 | 0 | 0 | 4.774 | 0 |
| TRACH1000193 | 0 | 0 | 0 | 15.417 | 0 | 0 | 0 |
| TRACH1000205 | 0 | 0 | 0 | 8.380 | 0 | 3.965 | 0 |
| TRACH2000045 | 0 | 0 | 0 | 19.517 | 0 | 8.312 | 0 |
| TRACH2000047 | 0 | 0 | 0 | 26.609 | 0 | 0 | 0 |
| TRACH2000079 | 0 | 0 | 0 | 0.769 | 0 | 3.275 | 0 |
| TRACH2000248 | 0 | 0 | 0 | 2.971 | 0 | 0.703 | 0 |
| TRACH2000321 | 0 | 0 | 0 | 1.744 | 1.775 | 0.495 | 0 |
| TRACH2000393 | 0 | 0 | 0 | 24.151 | 0 | 0 | 0 |
| TRACH2000420 | 0 | 0 | 0 | 0 | 0 | 3.379 | 0 |
| TRACH2000461 | 0 | 0 | 0 | 2.434 | 0 | 6.910 | 0 |
| TRACH2000472 | 0 | 0 | 0 | 0 | 0 | 0.860 | 0 |
| TRACH2000660 | 0 | 0 | 0 | 27.005 | 0 | 0 | 0 |
| TRACH2000677 | 0 | 0 | 0 | 40.018 | 0 | 28.405 | 0 |
| TRACH2000702 | 0 | 0 | 0 | 5.573 | 0 | 0 | 0 |
| TRACH2000780 | 0 | 0 | 0 | 0.620 | 0 | 2.642 | 0 |
| TRACH2000873 | 0 | 0 | 0 | 0 | 0 | 47.355 | 0 |
| TRACH2000920 | 0 | 0 | 0 | 4.706 | 0 | 0 | 0 |
| TRACH2000926 | 0 | 0 | 0 | 1.980 | 0 | 0 | 0 |
| TRACH2000959 | 0 | 0 | 0 | 0.347 | 0 | 0.492 | 92.224 |
| TRACH2000972 | 0 | 4.160 | 0 | 0.421 | 8.566 | 0.597 | 0 |
| TRACH2001021 | 0 | 5.878 | 0 | 0 | 1.513 | 0 | 0 |
| TRACH2001109 | 0 | 0 | 0 | 2.080 | 6.050 | 13.074 | 0 |
| TRACH2001249 | 0 | 0 | 0 | 0 | 0 | 9.240 | 0 |
| TRACH2001289 | 0 | 0 | 0 | 0.241 | 1.228 | 0.685 | 0 |
| TRACH2001314 | 0 | 0 | 0 | 3.892 | 0 | 1.842 | 0 |
| TRACH2001341 | 0 | 44.736 | 0 | 0 | 0 | 0 | 0 |
| TRACH2001432 | 0 | 7.808 | 0 | 0 | 8.039 | 1.121 | 0 |
| TRACH2001478 | 0 | 0 | 0 | 1.648 | 2.098 | 2.340 | 0 |
| TRACH2001523 | 0 | 0 | 0 | 2.413 | 0 | 0 | 0 |
| TRACH2001549 | 0 | 0 | 0 | 0 | 0 | 4.260 | 0 |
| TRACH2001607 | 0 | 0 | 23.236 | 3.564 | 0 | 2.024 | 0 |
| TRACH2001612 | 0 | 28.037 | 0 | 2.835 | 0 | 0 | 0 |
| TRACH2001646 | 0 | 0 | 0 | 0 | 0 | 22.786 | 0 |
| TRACH2001684 | 0 | 0 | 0 | 0 | 0 | 17.948 | 0 |
| TRACH2001810 | 0 | 0 | 0 | 1.102 | 2.806 | 0.782 | 0 |
| TRACH2002537 | 0 | 0 | 0 | 1.975 | 3.351 | 0 | 0 |
| TRACH2002664 | 0 | 42.726 | 0 | 1.080 | 0 | 0 | 0 |
| TRACH2002803 | 0 | 0 | 0 | 2.958 | 0 | 4.199 | 0 |
| TRACH2003272 | 0 | 0 | 0 | 2.630 | 0 | 11.200 | 0 |
| TRACH2003323 | 0 | 0 | 0 | 0 | 0 | 7.173 | 0 |
| TRACH2003347 | 0 | 0 | 0 | 0 | 0 | 1.601 | 0 |
| TRACH2003484 | 0 | 0 | 0 | 38.787 | 0 | 0 | 0 |
| TRACH2004399 | 0 | 0 | 0 | 42.079 | 0 | 0 | 0 |
| TRACH2004458 | 0 | 0 | 0 | 0 | 76.336 | 0 | 0 |
| TRACH2005800 | 0 | 0 | 0 | 0 | 0 | 1.212 | 0 |
| TRACH2007399 | 0 | 0 | 0 | 0.334 | 0 | 0 | 0 |
| TRACH2008113 | 0 | 0 | 0 | 13.211 | 0 | 0 | 0 |
| TRACH2008300 | 0 | 0 | 0 | 3.615 | 6.135 | 11.974 | 0 |
| TRACH2008472 | 0 | 0 | 0 | 1.114 | 0 | 0 | 0 |
| TRACH2008716 | 0 | 0 | 0 | 0 | 0 | 5.111 | 0 |
| TRACH2008891 | 0 | 0 | 0 | 15.398 | 0 | 0 | 0 |
| TRACH2009006 | 0 | 0 | 0 | 0 | 0 | 3.536 | 0 |
| TRACH2010824 | 0 | 0 | 0 | 0 | 64.903 | 0 | 0 |
| TRACH2011113 | 0 | 0 | 0 | 3.351 | 0 | 0 | 0 |
| TRACH2011894 | 0 | 0 | 0 | 0 | 0 | 8.960 | 0 |
| TRACH2012376 | 0 | 18.838 | 0 | 0 | 0 | 0 | 0 |
| TRACH2012811 | 0 | 0 | 0 | 5.533 | 28.170 | 0 | 0 |
| TRACH2013552 | 0 | 0 | 0 | 7.374 | 0 | 0 | 0 |
| TRACH2014077 | 0 | 0 | 0 | 2.414 | 0 | 0 | 0 |
| TRACH2014124 | 0 | 0 | 0 | 1.465 | 0 | 4.160 | 0 |
| TRACH2014997 | 0 | 0 | 0 | 12.555 | 0 | 0 | 0 |
| TRACH2015823 | 0 | 0 | 0 | 3.105 | 0 | 0 | 0 |
| TRACH2016533 | 0 | 0 | 0 | 10.173 | 0 | 0 | 0 |
| TRACH2018950 | 0 | 0 | 0 | 5.331 | 0 | 0 | 0 |
| TRACH2025344 | 0 | 69.123 | 0 | 0 | 0 | 19.846 | 0 |
| TRACH2025535 | 0 | 0 | 4.477 | 2.472 | 0 | 0 | 0 |
| TRACH2025911 | 0 | 0 | 0 | 0 | 0 | 47.355 | 0 |
| TRACH3002192 | 0 | 0 | 0 | 3.541 | 2.253 | 1.885 | 0 |
| TRACH3002752 | 0 | 35.850 | 0 | 3.625 | 0 | 0 | 0 |
| TRACH3003547 | 0 | 0 | 0 | 3.002 | 15.280 | 0 | 0 |
| TRACH3003872 | 0 | 0 | 0 | 0 | 0 | 2.765 | 0 |
| TRACH3004068 | 0 | 0 | 0 | 0 | 5.150 | 1.436 | 0 |
| TRACH3004721 | 0 | 0 | 0 | 4.127 | 0 | 5.858 | 0 |
| TRACH3006038 | 0 | 0 | 0 | 38.787 | 0 | 0 | 0 |
| TRACH3006412 | 0 | 0 | 0 | 38.787 | 0 | 0 | 0 |
| TRACH3006717 | 0 | 0 | 0 | 10.418 | 0 | 3.697 | 0 |
| TRACH3007479 | 0 | 0 | 0 | 0 | 0 | 1.084 | 0 |
| TRACH3007625 | 0 | 0 | 0 | 2.503 | 0 | 3.553 | 0 |
| TRACH3007995 | 0 | 0 | 0 | 38.787 | 0 | 0 | 0 |
| TRACH3008093 | 0 | 0 | 0 | 3.477 | 0 | 0 | 0 |
| TRACH3008632 | 0 | 0 | 0 | 11.995 | 0 | 0 | 0 |
| TRACH3009001 | 0 | 0 | 0 | 0 | 0 | 23.608 | 0 |
| TRACH3009956 | 0 | 0 | 0 | 55.894 | 0 | 0 | 0 |
| TRACH3013558 | 0 | 0 | 0 | 38.787 | 0 | 0 | 0 |
| TRACH3016805 | 0 | 0 | 0 | 3.196 | 0 | 0 | 0 |
| TRACH3019290 | 0 | 0 | 0 | 0 | 0 | 27.871 | 0 |
| TRACH3036932 | 0 | 0 | 0 | 10.405 | 0 | 0 | 0 |
| TRACH3036942 | 0 | 0 | 0 | 0 | 0 | 0 | 92.165 |
| TRACH3037067 | 0 | 0 | 0 | 0 | 0 | 1.620 | 0 |
| TRACH3038399 | 0 | 0 | 0 | 8.826 | 0 | 0 | 0 |
| TSTOM2000553 | 0 | 11.832 | 0 | 0 | 0 | 0 | 0 |
| TUTER2000904 | 0 | 0 | 0 | 5.944 | 0 | 2.813 | 0 |
| TUTER2002323 | 0 | 0 | 0 | 2.312 | 0 | 0 | 0 |
| UMVEN1000122 | 0 | 6.860 | 0 | 0 | 0 | 0 | 0 |
| UMVEN2000133 | 0 | 0 | 0 | 0 | 0 | 0.591 | 0 |
| UMVEN2000152 | 0 | 0 | 0 | 0.559 | 0 | 0 | 0 |
| UMVEN2000453 | 0 | 0 | 0 | 0.371 | 0 | 1.052 | 0 |
| UTERU1000182 | 0 | 0 | 0 | 0.576 | 2.931 | 0.817 | 0 |
| UTERU1000384 | 0 | 0 | 0 | 5.059 | 0 | 0 | 0 |
| UTERU2000023 | 0 | 0 | 0 | 29.063 | 0 | 10.315 | 0 |
| UTERU2000074 | 0 | 0 | 0 | 37.308 | 0 | 0 | 0 |
| UTERU2000095 | 0 | 2.905 | 3.192 | 0.881 | 0 | 2.919 | 0 |
| UTERU2000263 | 0 | 0 | 0 | 0 | 1.851 | 0 | 0 |
| UTERU2000424 | 0 | 5.961 | 0 | 3.014 | 3.069 | 0.856 | 0 |
| UTERU2001607 | 0 | 0 | 0 | 2.094 | 0 | 0 | 0 |
| UTERU2002410 | 0 | 0 | 0 | 0.417 | 0 | 0.592 | 0 |
| UTERU2002662 | 0 | 0 | 0 | 0 | 0 | 1.793 | 0 |
| UTERU2002826 | 0 | 0 | 0 | 0 | 13.337 | 0 | 0 |
| UTERU2003057 | 0 | 0 | 0 | 1.836 | 0 | 0 | 0 |
| UTERU2003456 | 0 | 0 | 0 | 0 | 0 | 10.897 | 0 |
| UTERU2003704 | 0 | 0 | 0 | 12.217 | 0 | 17.343 | 0 |
| UTERU2004197 | 0 | 0 | 0 | 1.427 | 0 | 4.052 | 0 |
| UTERU2004664 | 0 | 0 | 26.172 | 0 | 0 | 1.710 | 0 |
| UTERU2004861 | 0 | 0 | 0 | 0 | 0 | 23.838 | 0 |
| UTERU2004929 | 0 | 0 | 0 | 0 | 0 | 11.697 | 0 |
| UTERU2005354 | 0 | 0 | 0 | 4.658 | 0 | 0 | 0 |
| UTERU2005446 | 0 | 0 | 0 | 0 | 75.184 | 0 | 0 |
| UTERU2005449 | 0 | 0 | 0 | 0 | 0 | 16.496 | 0 |
| UTERU2005593 | 0 | 0 | 0 | 1.593 | 0 | 3.393 | 0 |
| UTERU2005621 | 0 | 0 | 0 | 0 | 8.145 | 0 | 0 |
| UTERU2006115 | 0 | 0 | 0 | 5.563 | 0 | 0 | 0 |
| UTERU2006593 | 0 | 0 | 0 | 1.048 | 0 | 5.951 | 0 |
| UTERU2007924 | 0 | 0 | 0 | 0 | 9.664 | 0 | 0 |
| UTERU2008040 | 0 | 0 | 0 | 8.353 | 0 | 11.858 | 0 |
| UTERU2008085 | 2.831 | 0 | 0.696 | 0.961 | 0.979 | 0.819 | 0 |
| UTERU2008302 | 0 | 0 | 0 | 0 | 0 | 4.038 | 0 |
| UTERU2008938 | 0 | 0 | 0 | 0 | 0 | 4.959 | 0 |
| UTERU2009335 | 0 | 0 | 0 | 0 | 0 | 3.056 | 0 |
| UTERU2009435 | 0 | 0 | 0 | 0 | 0 | 5.388 | 0 |
| UTERU2009510 | 0 | 0 | 0 | 0 | 54.308 | 0 | 0 |
| UTERU2010226 | 0 | 0 | 0 | 5.599 | 0 | 0 | 0 |
| UTERU2010417 | 0 | 2.213 | 0 | 0.224 | 0 | 1.271 | 0 |
| UTERU2010747 | 0 | 0 | 0 | 37.308 | 0 | 0 | 0 |
| UTERU2011220 | 0 | 0 | 0 | 1.389 | 0 | 0 | 0 |
| UTERU2011621 | 0 | 0 | 0 | 28.191 | 0 | 0 | 0 |
| UTERU2011906 | 0 | 0 | 0 | 0 | 0 | 13.742 | 0 |
| UTERU2012230 | 0 | 0 | 0 | 9.992 | 0 | 1.773 | 0 |
| UTERU2012286 | 0 | 0 | 0 | 1.798 | 0 | 0 | 0 |
| UTERU2012407 | 0 | 0 | 0 | 0 | 0 | 24.800 | 0 |
| UTERU2012526 | 0 | 0 | 0 | 0 | 66.732 | 0 | 0 |
| UTERU2012856 | 0 | 0 | 0 | 2.504 | 0 | 7.110 | 0 |
| UTERU2014024 | 0 | 0 | 0 | 9.198 | 0 | 0 | 0 |
| UTERU2014167 | 0 | 0 | 0 | 0 | 0 | 0.918 | 0 |
| UTERU2014601 | 0 | 0 | 0 | 4.916 | 0 | 13.956 | 0 |
| UTERU2014898 | 0 | 0 | 0 | 2.160 | 0 | 0 | 0 |
| UTERU2016799 | 0 | 20.409 | 0 | 8.255 | 0 | 0 | 0 |
| UTERU2016822 | 0 | 0 | 0 | 0 | 0 | 4.050 | 0 |
| UTERU2017761 | 0 | 0 | 0 | 37.308 | 0 | 0 | 0 |
| UTERU2018200 | 0 | 0 | 0 | 17.226 | 0 | 0 | 0 |
| UTERU2018566 | 0 | 0 | 0 | 12.651 | 0 | 0 | 0 |
| UTERU2018712 | 0 | 0 | 0 | 0 | 0 | 45.794 | 0 |
| UTERU2018884 | 0 | 0 | 0 | 1.100 | 0 | 1.561 | 0 |
| UTERU2019005 | 0 | 0 | 0 | 4.584 | 0 | 6.508 | 0 |
| UTERU2019096 | 0 | 0 | 0 | 4.905 | 8.323 | 2.321 | 0 |
| UTERU2021820 | 0 | 0 | 0 | 17.694 | 0 | 0 | 0 |
| UTERU2024969 | 0 | 22.540 | 0 | 0 | 0 | 0 | 0 |
| UTERU2025301 | 0 | 0 | 0 | 3.085 | 0 | 0 | 0 |
| UTERU3000226 | 0 | 0 | 0 | 37.308 | 0 | 0 | 0 |
| UTERU3000298 | 0 | 0 | 0 | 13.135 | 0 | 13.985 | 0 |
| UTERU3000670 | 0 | 0 | 0 | 16.553 | 0 | 0 | 0 |
| UTERU3000727 | 0 | 0 | 0 | 32.047 | 0 | 0 | 0 |
| UTERU3001158 | 0 | 0 | 0 | 23.569 | 0 | 0 | 0 |
| UTERU3001766 | 0 | 0 | 0 | 0 | 0 | 45.794 | 0 |
| UTERU3003660 | 0 | 0 | 0 | 5.226 | 0 | 14.837 | 0 |
| UTERU3005230 | 0 | 0 | 0 | 17.332 | 0 | 0 | 0 |
| UTERU3005460 | 0 | 0 | 0 | 37.308 | 0 | 0 | 0 |
| UTERU3005536 | 0 | 0 | 0 | 37.308 | 0 | 0 | 0 |
| UTERU3005767 | 0 | 8.354 | 0 | 0 | 0 | 2.398 | 0 |
| UTERU3005970 | 0 | 0 | 0 | 37.308 | 0 | 0 | 0 |
| UTERU3006308 | 0 | 0 | 0 | 37.308 | 0 | 0 | 0 |
| UTERU3007419 | 0 | 0 | 0 | 37.308 | 0 | 0 | 0 |
| UTERU3007913 | 0 | 0 | 0 | 12.951 | 0 | 0 | 0 |
| UTERU3009517 | 0 | 0 | 0 | 0 | 0 | 45.794 | 0 |
| UTERU3010409 | 0 | 0 | 0 | 17.196 | 0 | 0 | 0 |
| UTERU3015011 | 0 | 0 | 0 | 5.009 | 0 | 0 | 0 |

Genes whose expression levels were negligibly low in each of the above-described libraries are shown with their clone names below.

3NB691000018, 3NB691000113, 3NB691000129, 3NB691000191, 3NB692000012, 3NB692000029, 3NB692000102, 3NB692000154, 3NB692000276, 3NB692000305, 3NB692000429, 3NB692000484, 3NB692000529, 3NB692000973, 3NB692001002, 3NB692001034, 3NB692001040, 3NB692001123, 3NB692001267, 3NB692001339, 3NB692001349, 3NB692001366, 3NB692001408, 3NB692001433, 3NB692001459, 3NB692001471, 3NB692001501, 3NB692001507, 3NB692001511, 3NB692001519, 3NB692001520, 3NB692001528, 3NB692001538, 3NB692001548, 3NB692001557, 3NB692001637, 3NB692001861, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064, ADIPS2000069, ADIPS2000088, ADIPS2000245, ADIPS2000425, ADRGL1000038, ADRGL1000067, ADRGL1000111, ADRGL1000144, ADRGL1000147, ADRGL1000160, ADRGL1000165, ADRGL1000182, ADRGL2000056, ADRGL2000064, ADRGL2000085, ADRGL2000117, ADRGL2000142, ADRGL2000172, ADRGL2000248, ADRGL2000261, ADRGL2000323, ADRGL2000353, ADRGL2000384, ADRGL2000428, ADRGL2000636, ADRGL2000968, ADRGL2001119, ADRGL2001229, ADRGL2001287, ADRGL2001301, ADRGL2001354, ADRGL2001554, ADRGL2001651, ADRGL2001756, ADRGL2001836, ADRGL2001854, ADRGL2002029, ADRGL2002260, ADRGL2002392, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007357, ADRGL2007636, ADRGL2007651, ADRGL2007810, ADRGL2007877, ADRGL2008023, ADRGL2008331, ADRGL2008337, ADRGL2008535, ADRGL2009324, ADRGL2009489, ADRGL2009533, ADRGL2009691, ADRGL2009755, ADRGL2010315, ADRGL2010594, ADRGL2010860, ADRGL2010974, ADRGL2011190, ADRGL2011330, ADRGL2012179, AHMSC1000138, ASTRO1000009, ASTRO1000018, ASTRO1000029, ASTRO1000096, ASTRO1000165, ASTRO2000011, ASTRO2000095, ASTRO2000191, ASTRO2000278, ASTRO2000372, ASTRO2000403, ASTRO2000417, ASTRO2000482, ASTRO2000533, ASTRO2000653, ASTRO2000662, ASTRO2000725, ASTRO2000801, ASTRO2000914, ASTRO2001001, ASTRO2001008, ASTRO2001076, ASTRO2001088, ASTRO2001122, ASTRO2001227, ASTRO2001249, ASTRO2001480, ASTRO2002035, ASTRO2002064, ASTRO2002202, ASTRO2002571, ASTRO2002632, ASTRO2002659, ASTRO2002720, ASTRO2002743, ASTRO2002842, ASTRO2003574, ASTRO2003581, ASTRO2003632, ASTRO2003740, ASTRO2003925, ASTRO2004051, ASTRO2004217, ASTRO2004297, ASTRO2004584, ASTRO2004628, ASTRO2004751, ASTRO2004877, ASTRO2005008, ASTRO2005081, ASTRO2005096, ASTRO2005242, ASTRO2005273, ASTRO2005343, ASTRO2005360, ASTRO2005413, ASTRO2005557, ASTRO2005575, ASTRO2005593, ASTRO2005687, ASTRO2005726, ASTRO2005863, ASTRO2005896, ASTRO2005902, ASTRO2006356, ASTRO2006732, ASTRO2006747, ASTRO2006920, ASTRO2006952, ASTRO2007221, ASTRO2007377, ASTRO2007515, ASTRO2007578, ASTRO2007643, ASTRO2007666, ASTRO2007956, ASTRO2007962, ASTRO2008040, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014923, ASTRO2014961, ASTRO2015162, ASTRO2015185, ASTRO2015529, ASTRO2015987, ASTRO2016044, ASTRO2016114, ASTRO2016148, ASTRO2016681, ASTRO2016847, ASTRO2016892, ASTRO2017348, ASTRO2017627, ASTRO2018373, ASTRO2019039, ASTRO3000154, ASTRO3000172, ASTRO3000177, ASTRO3000301, ASTRO3000474, ASTRO3000482, BEAST2000454, BRAWH1000002, BRAWH1000007, BRAWH1000116, BRAWH1000130, BRAWH1000157, BRAWH1000164, BRAWH1000168, BRAWH1000180, BRAWH1000369, BRAWH2000034, BRAWH2000048, BRAWH2000071, BRAWH2000093, BRAWH2000109, BRAWH2000142, BRAWH2000169, BRAWH2000218, BRAWH2000232, BRAWH2000248, BRAWH2000256, BRAWH2000323, BRAWH2000370, BRAWH2000409, BRAWH2000471, BRAWH2000476, BRAWH2000488, BRAWH2000503, BRAWH2000554, BRAWH2000588, BRAWH2000643, BRAWH2000651, BRAWH2000682, BRAWH2000752, BRAWH2000859, BRAWH2000866, BRAWH2000892, BRAWH2000901, BRAWH2000926, BRAWH2000944, BRAWH2000984, BRAWH2001052, BRAWH2001103, BRAWH2001129, BRAWH2001137, BRAWH2001159, BRAWH2001203, BRAWH2001239, BRAWH2001255, BRAWH2001355, BRAWH2001378, BRAWH2001406, BRAWH2001412, BRAWH2001424, BRAWH2001436, BRAWH2001438, BRAWH2001484, BRAWH2001492, BRAWH2001503, BRAWH2001519, BRAWH2001530, BRAWH2001589, BRAWH2001666, BRAWH2001679, BRAWH2001686, BRAWH2001701, BRAWH2001842, BRAWH2001862, BRAWH2001873, BRAWH2001940, BRAWH2001973, BRAWH2002062, BRAWH2002125, BRAWH2002191, BRAWH2002333, BRAWH2002352, BRAWH2002383, BRAWH2002623, BRAWH2002630, BRAWH2002635, BRAWH2002662, BRAWH2002679, BRAWH2002725, BRAWH2002761, BRAWH2002861, BRAWH2002967, BRAWH2002976, BRAWH2003135, BRAWH2003240, BRAWH2003307, BRAWH2003366, BRAWH2003689, BRAWH2003693, BRAWH2003818, BRAWH2003832, BRAWH2003948, BRAWH2003964, BRAWH2004044, BRAWH2004068, BRAWH2004136, BRAWH2004217, BRAWH2004580, BRAWH2004731, BRAWH2004884, BRAWH2004923, BRAWH2005074, BRAWH2005225, BRAWH2005315, BRAWH2005517, BRAWH2005524, BRAWH2005533, BRAWH2005578, BRAWH2005812, BRAWH2005974, BRAWH2005998, BRAWH2006083, BRAWH2006395, BRAWH2006405, BRAWH2006450, BRAWH2006526, BRAWH2006760, BRAWH2006960, BRAWH2007069, BRAWH2007308, BRAWH2007406, BRAWH2007550, BRAWH2007570, BRAWH2007658, BRAWH2007800, BRAWH2007808, BRAWH2007862, BRAWH2007890, BRAWH2008058, BRAWH2008128, BRAWH2008219, BRAWH2008292, BRAWH2008297, BRAWH2008366, BRAWH2008380, BRAWH2008540, BRAWH2008577, BRAWH2008790, BRAWH2009112, BRAWH2009227, BRAWH2009261, BRAWH2009263, BRAWH2009298, BRAWH2009304, BRAWH2009360, BRAWH2009393, BRAWH2009485, BRAWH2009490, BRAWH2009558, BRAWH2009590, BRAWH2009626, BRAWH2009678, BRAWH2009907, BRAWH2010000, BRAWH2010069, BRAWH2010084, BRAWH2010318, BRAWH2010536, BRAWH2010584, BRAWH2010618, BRAWH2010619, BRAWH2010980, BRAWH2011066, BRAWH2011079, BRAWH2011096, BRAWH2011155, BRAWH2011166, BRAWH2011286, BRAWH2011294, BRAWH2011400, BRAWH2011762, BRAWH2011795, BRAWH2011796, BRAWH2011823, BRAWH2011860, BRAWH2011958, BRAWH2012054, BRAWH2012077, BRAWH2012161, BRAWH2012162, BRAWH2012258, BRAWH2012277, BRAWH2012326, BRAWH2012540, BRAWH2012619, BRAWH2012749, BRAWH2012963, BRAWH2013866, BRAWH2013871, BRAWH2013914, BRAWH2013941, BRAWH2013955, BRAWH2014234, BRAWH2014379, BRAWH2014473, BRAWH2014717, BRAWH2014729, BRAWH2014934, BRAWH2015349, BRAWH2015375, BRAWH2015595, BRAWH2015853, BRAWH2015866, BRAWH2016106, BRAWH2016160, BRAWH2016221, BRAWH2016223, BRAWH2016305, BRAWH2016439, BRAWH2016446, BRAWH2016514, BRAWH2016562, BRAWH2016655, BRAWH2016724, BRAWH2016785, BRAWH2016792, BRAWH2016800, BRAWH2017103, BRAWH2017250, BRAWH2017294, BRAWH2017305, BRAWH2017407, BRAWH2017433, BRAWH2017523, BRAWH2017534, BRAWH2017542, BRAWH2017635, BRAWH2017913, BRAWH2018206, BRAWH2018282, BRAWH2018317, BRAWH2018506, BRAWH2018514, BRAWH2018527, BRAWH2018601, BRAWH2018729, BRAWH2018745, BRAWH2019053, BRAWH2019055, BRAWH2019198, BRAWH3000078, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001326, BRAWH3001475, BRAWH3001636, BRAWH3001638, BRAWH3001712, BRAWH3001760, BRAWH3001783, BRAWH3001833, BRAWH3001879, BRAWH3002433, BRAWH3002467, BRAWH3002513, BRAWH3002574, BRAWH3002819, BRAWH3003019, BRAWH3003136, BRAWH3003244, BRAWH3003343, BRAWH3003411, BRAWH3003522, BRAWH3003598, BRAWH3003801, BRAWH3003975, BRAWH3004222, BRAWH3004335, BRAWH3004350, BRAWH3004453, BRAWH3004666, BRAWH3005037, BRAWH3005047, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006792, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3009017, BRAWH3009546, BRAWH3009961, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016715, BRAWH3017260, BRAWH3017477, BRAWH3017537, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3030613, BRAWH3030772, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034775, BRAWH3034890, BRAWH3035154, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000105, BRCAN1000149, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001866, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006051, BRCAN2006063, BRCAN2006174, BRCAN2006290, BRCAN2006297, BRCAN2006323, BRCAN2006411, BRCAN2006450, BRCAN2006955, BRCAN2007119, BRCAN2007144, BRCAN2007409, BRCAN2007426, BRCAN2007525, BRCAN2008494, BRCAN2008528, BRCAN2008701, BRCAN2009156, BRCAN2009168, BRCAN2009203, BRCAN2010547, BRCAN2010581, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014952, BRCAN2015402, BRCAN2015464, BRCAN2015757, BRCAN2015886, BRCAN2016025, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019773, BRCAN2019907, BRCAN2020234, BRCAN2020331, BRCAN2020412, BRCAN2020467, BRCAN2020880, BRCAN2021024, BRCAN2021028, BRCAN2021325, BRCAN2021452, BRCAN2021669, BRCAN2021718, BRCAN2022191, BRCAN2022472, BRCAN2022474, BRCAN2023347, BRCAN2023724, BRCAN2024451, BRCAN2024563, BRCAN2025009, BRCAN2025093, BRCAN2025349, BRCAN2025712, BRCAN2025982, BRCAN2026117, BRCAN2026711, BRCAN2027150, BRCAN2027310, BRCAN2027355, BRCAN2027513, BRCAN2027593, BRCAN2027970, BRCAN2028021, BRCAN2028040, BRCAN2028319, BRCAN2028338, BRCAN2028378, BRCAN2028460, BRCAN2028545, BRCAN2028634, BRCAN2028702, BRCOC1000087, BRCOC2000152, BRCOC2000184, BRCOC2000360, BRCOC2000404, BRCOC2000487, BRCOC2000637, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003125, BRCOC2003513, BRCOC2003732, BRCOC2003740, BRCOC2004175, BRCOC2004354, BRCOC2004455, BRCOC2004950, BRCOC2005951, BRCOC2006164, BRCOC2006243, BRCOC2006639, BRCOC2006942, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2010115, BRCOC2010123, BRCOC2010510, BRCOC2010730, BRCOC2010980, BRCOC2010994, BRCOC2011010, BRCOC2011398, BRCOC2011418, BRCOC2011769, BRCOC2011996, BRCOC2012229, BRCOC2012386, BRCOC2012813, BRCOC2013448, BRCOC2013573, BRCOC2013675, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019903, BRCOC2019934, BRCOC2020122, BRCOC2020142, BRHIP1000058, BRHIP1000072, BRHIP1000258, BRHIP2000021, BRHIP2000079, BRHIP2000163, BRHIP2000239, BRHIP2000245, BRHIP2000359, BRHIP2000445, BRHIP2000506, BRHIP2000532, BRHIP2000621, BRHIP2000691, BRHIP2000819, BRHIP2000826, BRHIP2001074, BRHIP2001686, BRHIP2001782, BRHIP2001802, BRHIP2001805, BRHIP2001927, BRHIP2002122, BRHIP2002172, BRHIP2002346, BRHIP2002510, BRHIP2002664, BRHIP2002722, BRHIP2002929, BRHIP2003048, BRHIP2003242, BRHIP2003272, BRHIP2003748, BRHIP2003786, BRHIP2003828, BRHIP2003917, BRHIP2004312, BRHIP2004355, BRHIP2004814, BRHIP2004883, BRHIP2004902, BRHIP2005236, BRHIP2005354, BRHIP2005600, BRHIP2005719, BRHIP2005724, BRHIP2005932, BRHIP2006617, BRHIP2006782, BRHIP2006800, BRHIP2006819, BRHIP2006921, BRHIP2007307, BRHIP2007616, BRHIP2007690, BRHIP2007928, BRHIP2007969, BRHIP2008269, BRHIP2008389, BRHIP2008756, BRHIP2008909, BRHIP2009177, BRHIP2009340, BRHIP2009351, BRHIP2009414, BRHIP2009474, BRHIP2009617, BRHIP2009685, BRHIP2010309, BRHIP2010444, BRHIP2010593, BRHIP2010610, BRHIP2010744, BRHIP2011080, BRHIP2011199, BRHIP2011508, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013723, BRHIP2013958, BRHIP2013972, BRHIP2014063, BRHIP2014285, BRHIP2014291, BRHIP2014373, BRHIP2014386, BRHIP2014387, BRHIP2014954, BRHIP2015356, BRHIP2015360, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017714, BRHIP2017780, BRHIP2018014, BRHIP2018369, BRHIP2018612, BRHIP2018635, BRHIP2018650, BRHIP2018805, BRHIP2018998, BRHIP2019007, BRHIP2019047, BRHIP2019149, BRHIP2019177, BRHIP2019291, BRHIP2019589, BRHIP2019641, BRHIP2019884, BRHIP2019972, BRHIP2020182, BRHIP2020509, BRHIP2020695, BRHIP2020743, BRHIP2020842, BRHIP2020859, BRHIP2020930, BRHIP2021102, BRHIP2021289, BRHIP2021615, BRHIP2021744, BRHIP2021929, BRHIP2022006, BRHIP2022221, BRHIP2022228, BRHIP2022298, BRHIP2022467, BRHIP2022708, BRHIP2023229, BRHIP2023272, BRHIP2023292, BRHIP2023369, BRHIP2023438, BRHIP2023695, BRHIP2023735, BRHIP2023773, BRHIP2023860, BRHIP2023869, BRHIP2024046, BRHIP2024165, BRHIP2024201, BRHIP2024347, BRHIP2024549, BRHIP2024789, BRHIP2024911, BRHIP2024941, BRHIP2025245, BRHIP2025366, BRHIP2025739, BRHIP2025797, BRHIP2026021, BRHIP2026155, BRHIP2026214, BRHIP2026346, BRHIP2026557, BRHIP2026723, BRHIP2026877, BRHIP2027017, BRHIP2027054, BRHIP2027563, BRHIP2027762, BRHIP2028303, BRHIP2028330, BRHIP2028480, BRHIP2028593, BRHIP2029176, BRHIP2029283, BRHIP2029393, BRHIP2029529, BRHIP2029663, BRHIP3000017, BRHIP3000060, BRHIP3000070, BRHIP3000081, BRHIP3000475, BRHIP3000488, BRHIP3000526, BRHIP3000626, BRHIP3000859, BRHIP3000907, BRHIP3001076, BRHIP3001079, BRHIP3001141, BRHIP3001338, BRHIP3001481, BRHIP3001878, BRHIP3001964, BRHIP3002124, BRHIP3002141, BRHIP3002363, BRHIP3002920, BRHIP3002931, BRHIP3003306, BRHIP3003340, BRHIP3003395, BRHIP3003484, BRHIP3003644, BRHIP3003688, BRHIP3003795, BRHIP3004193, BRHIP3004416, BRHIP3004710, BRHIP3004725, BRHIP3004774, BRHIP3004786, BRHIP3004863, BRHIP3005037, BRHIP3005137, BRHIP3005142, BRHIP3005231, BRHIP3005307, BRHIP3005768, BRHIP3005944, BRHIP3006279, BRHIP3006294, BRHIP3006390, BRHIP3006449, BRHIP3006683, BRHIP3006786, BRHIP3006950, BRHIP3007172, BRHIP3007183, BRHIP3007195, BRHIP3007239, BRHIP3007291, BRHIP3007409, BRHIP3007424, BRHIP3007483, BRHIP3007609, BRHIP3007960, BRHIP3008082, BRHIP3008183, BRHIP3008314, BRHIP3008320, BRHIP3008344, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3015620, BRHIP3015751, BRHIP3015854, BRHIP3016032, BRHIP3016213, BRHIP3016302, BRHIP3016421, BRHIP3017109, BRHIP3017256, BRHIP3017325, BRHIP3017558, BRHIP3017637, BRHIP3018784, BRHIP3019643, BRHIP3019824, BRHIP3019880, BRHIP3019956, BRHIP3020155, BRHIP3020182, BRHIP3020733, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026097, BRHIP3026231, BRHIP3026335, BRHIP3026651, BRHIP3027105, BRHIP3027137, BRHIP3027160, BRHIP3027191, BRHIP3027501, BRHIP3027651, BRHIP3027854, BRHIP3027947, BRHIP3028570, BRHIP3028742, BRSSN1000056, BRSSN1000059, BRSSN1000092, BRSSN1000153, BRSSN2000175, BRSSN2000197, BRSSN2000244, BRSSN2000518, BRSSN2000684, BRSSN2000715, BRSSN2001213, BRSSN2001275, BRSSN2001342, BRSSN2001426, BRSSN2001503, BRSSN2001579, BRSSN2001810, BRSSN2002857, BRSSN2002859, BRSSN2003086, BRSSN2003093, BRSSN2003820, BRSSN2003841, BRSSN2003937, BRSSN2004142, BRSSN2004303, BRSSN2004304, BRSSN2004657, BRSSN2004679, BRSSN2004686, BRSSN2004710, BRSSN2004719, BRSSN2005847, BRSSN2006133, BRSSN2006390, BRSSN2006575, BRSSN2006644, BRSSN2007076, BRSSN2007168, BRSSN2007202, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009627, BRSSN2009630, BRSSN2009702, BRSSN2010019, BRSSN2010110, BRSSN2010830, BRSSN2011653, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013702, BRSSN2013753, BRSSN2013874, BRSSN2014112, BRSSN2014424, BRSSN2014513, BRSSN2014556, BRSSN2014573, BRSSN2014636, BRSSN2015369, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018581, BRSSN2018731, BRSSN2018771, BRSSN2018925, BRSTN1000097, BRSTN2000070, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006583, BRSTN2006638, BRSTN2006865, BRSTN2007000, BRSTN2007118, BRSTN2007284, BRSTN2007765, BRSTN2008283, BRSTN2008367, BRSTN2008418, BRSTN2008457, BRSTN2008475, BRSTN2008930, BRSTN2009247, BRSTN2010363, BRSTN2010416, BRSTN2010500, BRSTN2010569, BRSTN2010923, BRSTN2011211, BRSTN2011688, BRSTN2011799, BRSTN2012284, BRSTN2012320, BRSTN2012380, BRSTN2013171, BRSTN2013354, BRSTN2014490, BRSTN2014633, BRSTN2014751, BRSTN2014770, BRSTN2016066, BRSTN2016335, BRSTN2016678, BRSTN2016892, BRSTN2016918, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000063, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005448, BRTHA2005579, BRTHA2005831, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2008316, BRTHA2008335, BRTHA2008502, BRTHA2008513, BRTHA2008527, BRTHA2008535, BRTHA2008598, BRTHA2008669, BRTHA2009311, BRTHA2009349, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011321, BRTHA2011351, BRTHA2011500, BRTHA2011691, BRTHA2012129, BRTHA2012183, BRTHA2012189, BRTHA2012351, BRTHA2012392, BRTHA2012466, BRTHA2012555, BRTHA2012562, BRTHA2012714, BRTHA2012980, BRTHA2013262, BRTHA2013275, BRTHA2013426, BRTHA2013460, BRTHA2013515, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026290, BRTHA2026311, BRTHA2027227, BRTHA2027229, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033264, BRTHA2033320, BRTHA2033469, BRTHA2033683, BRTHA2034281, BRTHA2034576, BRTHA2034874, BRTHA2035448, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3001221, BRTHA3001623, BRTHA3002265, BRTHA3002401, BRTHA3002411, BRTHA3002864, BRTHA3002933, BRTHA3002955, BRTHA3003000, BRTHA3003074, BRTHA3003225, BRTHA3003339, BRTHA3003343, BRTHA3003417, BRTHA3003449, BRTHA3003474, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009291, BRTHA3010135, BRTHA3010366, BRTHA3010469, BRTHA3010540, BRTHA3010717, BRTHA3011149, BRTHA3011187, BRTHA3011194, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021786, BRTHA3022641, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026556, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000797, CHONS2001287, CHONS2001382, CHONS2001797, CHONS2001834, CHONS2002419, COLON1000030, COLON1000084, COLON1000135, COLON2000557, COLON2000568, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON20.07733, COLON2008105, COLON2009337, COLON2009499, CORDB1000140, CORDB2000061, CORDB2000127, CORDB2000541, CTONG1000009, CTONG1000020, CTONG1000022, CTONG1000040, CTONG1000056, CTONG1000062, CTONG1000086, CTONG1000093, CTONG1000097, CTONG1000113, CTONG1000137, CTONG1000165, CTONG1000180, CTONG1000181, CTONG1000220, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042, CTONG2000218, CTONG2000279, CTONG2000303, CTONG2000330, CTONG2000395, CTONG2000411, CTONG2000452, CTONG2000465, CTONG2000469, CTONG2000480, CTONG2000561, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001366, CTONG2001413, CTONG2001428, CTONG2001513, CTONG2001524, CTONG2001533, CTONG2001681, CTONG2001724, CTONG2001749, CTONG2001800, CTONG2001820, CTONG2001858, CTONG2001877, CTONG2001911, CTONG2001932, CTONG2001955, CTONG2002066, CTONG2002073, CTONG2002095, CTONG2002143, CTONG2002395, CTONG2002417, CTONG2002418, CTONG2002453, CTONG2002558, CTONG2002590, CTONG2002721, CTONG2002816, CTONG2002820, CTONG2002832, CTONG2002903, CTONG2002965, CTONG2002999, CTONG2003028, CTONG2003100, CTONG2003115, CTONG2003189, CTONG2003245, CTONG2003293, CTONG2003348, CTONG2003350, CTONG2003361, CTONG2003372, CTONG2003375, CTONG2003427, CTONG2003517, CTONG2003599, CTONG2003680, CTONG2003764, CTONG2003782, CTONG2003889, CTONG2003937, CTONG2003945, CTONG2004000, CTONG2004062, CTONG2004126, CTONG2004264, CTONG2004305, CTONG2004397, CTONG2004454, CTONG2004550, CTONG2004669, CTONG2004716, CTONG2004912, CTONG2004941, CTONG2004948, CTONG2005049, CTONG2005110, CTONG2005145, CTONG2005265, CTONG2005278, CTONG2005290, CTONG2005374, CTONG2005399, CTONG2005468, CTONG2005567, CTONG2005585, CTONG2005635, CTONG2005775, CTONG2005795, CTONG2005890, CTONG2005904, CTONG2005913, CTONG2006129, CTONG2006223, CTONG2006273, CTONG2006310, CTONG2006377, CTONG2006393, CTONG2006412, CTONG2006449, CTONG2006515, CTONG2006524, CTONG2006568, CTONG2006620, CTONG2006666, CTONG2006691, CTONG2006798, CTONG2006836, CTONG2006942, CTONG2006964, CTONG2007009, CTONG2007091, CTONG2007104, CTONG2007175, CTONG2007259, CTONG2007272, CTONG2007293, CTONG2007297, CTONG2007399, CTONG2007417, CTONG2007441, CTONG2007586, CTONG2007613, CTONG2007623, CTONG2007779, CTONG2007898, CTONG2007959, CTONG2007970, CTONG2008014, CTONG2008184, CTONG2008262, CTONG2008269, CTONG2008321, CTONG2008343, CTONG2008402, CTONG2008466, CTONG2008518, CTONG2008521, CTONG2008562, CTONG2008577, CTONG2008595, CTONG2008621, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010508, CTONG2010566, CTONG2010623, CTONG2010649, CTONG2010652, CTONG2011188, CTONG2011429, CTONG2011474, CTONG2011676, CTONG2011716, CTONG2011765, CTONG2011779, CTONG2011801, CTONG2011815, CTONG2011822, CTONG2011920, CTONG2011985, CTONG2012029, CTONG2012050, CTONG2012077, CTONG2012123, CTONG2012158, CTONG2012230, CTONG2012422, CTONG2012425, CTONG2012447, CTONG2012473, CTONG2012476, CTONG2012533, CTONG2012554, CTONG2012564, CTONG2012607, CTONG2012661, CTONG2012724, CTONG2012738, CTONG2012758, CTONG2012843, CTONG2012901, CTONG2012996, CTONG2013128, CTONG2013149, CTONG2013156, CTONG2013178, CTONG2013222, CTONG2013284, CTONG2013339, CTONG2013352, CTONG2013381, CTONG2013393, CTONG2013511, CTONG2013630, CTONG2013803, CTONG2013985, CTONG2013986, CTONG2014032, CTONG2014058, CTONG2014119, CTONG2014165, CTONG2014191, CTONG2014206, CTONG2014234, CTONG2014264, CTONG2014389, CTONG2014491, CTONG2014630, CTONG2014635, CTONG2014646, CTONG2014705, CTONG2014946, CTONG2014954, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015866, CTONG2015914, CTONG2015953, CTONG2016056, CTONG2016217, CTONG2016355, CTONG2016408, CTONG2016499, CTONG2016505, CTONG2016559, CTONG2016658, CTONG2016775, CTONG2016797, CTONG2016846, CTONG2016904, CTONG2016942, CTONG2016953, CTONG2017021, CTONG2017292, CTONG2017409, CTONG2017429, CTONG2017444, CTONG2017458, CTONG2017604, CTONG2017650, CTONG2017798, CTONG2017804, CTONG2017814, CTONG2017910, CTONG2017939, CTONG2017989, CTONG2018062, CTONG2018069, CTONG2018147, CTONG2018217, CTONG2018279, CTONG2018334, CTONG2018379, CTONG2018383, CTONG2018413, CTONG2018483, CTONG2018629, CTONG2018652, CTONG2018653, CTONG2018655, CTONG2018735, CTONG2018808, CTONG2018900, CTONG2018976, CTONG2019063, CTONG2019144, CTONG2019232, CTONG2019248, CTONG2019400, CTONG2019590, CTONG2019652, CTONG2019704, CTONG2019788, CTONG2019822, CTONG2019833, CTONG2020031, CTONG2020108, CTONG2020127, CTONG2020171, CTONG2020378, CTONG2020411, CTONG2020445, CTONG2020522, CTONG2020560, CTONG2020638, CTONG2020806, CTONG2020831, CTONG2020974, CTONG2021024, CTONG2021132, CTONG2021168, CTONG2022153, CTONG2022601, CTONG2023021, CTONG2023449, CTONG2023512, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027327, CTONG2027361, CTONG2027591, CTONG2027736, CTONG2027783, CTONG2027828, CTONG2028097, CTONG2028486, CTONG2028518, CTONG2028687, CTONG2028758, CTONG3000686, CTONG3000707, CTONG3000896, CTONG3001123, CTONG3001420, CTONG3001501, CTONG3001560, CTONG3001605, CTONG3002020, CTONG3002127, CTONG3002370, CTONG3002412, CTONG3002433, CTONG3002518, CTONG3002552, CTONG3002588, CTONG3002674, CTONG3002728, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009028, CTONG3009227, CTONG3009287, CTONG3009385, D3OST1000066, D3OST1000109, D3OST1000238, D3OST1000267, D3OST2000184, D3OST2000618, D3OST2000654, D3OST2000734, D3OST2001328, D3OST2001669, D3OST2001670, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000507, D9OST2000031, D9OST2000278, D9OST2000440, D9OST2000869, D9OST2001319, D9OST2001433, D9OST2002397, D9OST2002673, D9OST2003106, D9OST2003137, D9OST2003397, D9OST2003594, D9OST2003762, D9OST2003791, D9OST2003989, D9OST2004018, D9OST2004073, D9OST2004417, DFNES1000003, DFNES1000107, DFNES1000150, DFNES1000185, DFNES2000011, DFNES2000143, DFNES2000146, DFNES2000153, DFNES2000292, DFNES2000426, DFNES2000432, DFNES2000443, DFNES2000471, DFNES2000913, DFNES2001091, DFNES2001108, DFNES2001177, DFNES2001404, DFNES2001564, DFNES2001565, DFNES2001647, DFNES2001800, DFNES2002220, DFNES2002509, DFNES2002550, DFNES2002588, DFNES2002817, DFNES2002966, DFNES2003081, DFNES2003255, DFNES2003742, DFNES2004346, DFNES2004354, DFNES2004383, DFNES2004608, DFNES2004684, DFNES2005266, DFNES2005527, DFNES2005766, DFNES2005779, DFNES2005875, DFNES2006346, DFNES2006944, DFNES2007113, DFNES2007299, DFNES2007332, DFNES2007641, DFNES2007757, DFNES2008280, DFNES2008881, DFNES2010502, DFNES2011192, DFNES2011221, DFNES2011239, DFNES2011245, ERLTF1000021, ERLTF2000324, ERLTF2000465, FCBBF1000023, FCBBF1000025, FCBBF1000038, FCBBF1000061, FCBBF1000069, FCBBF1000117, FCBBF1000118, FCBBF1000131, FCBBF1000155, FCBBF1000157, FCBBF1000171, FCBBF1000174, FCBBF1000182, FCBBF1000198, FCBBF1000206, FCBBF1000220, FCBBF1000232, FCBBF1000233, FCBBF1000243, FCBBF1000270, FCBBF1000272, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000412, FCBBF1000425, FCBBF1000437, FCBBF1000449, FCBBF1000506, FCBBF1000546, FCBBF1000550, FCBBF1000581, FCBBF1000582, FCBBF1000598, FCBBF1000604, FCBBF1000618, FCBBF1000627, FCBBF1000684, FCBBF1000686, FCBBF1000691, FCBBF1000748, FCBBF2000087, FCBBF2000094, FCBBF2000105, FCBBF2000123, FCBBF2000195, FCBBF2000214, FCBBF2000232, FCBBF2000259, FCBBF2000276, FCBBF2000291, FCBBF2000379, FCBBF2000473, FCBBF2000502, FCBBF2000576, FCBBF2000591, FCBBF2000677, FCBBF2000678, FCBBF2000685, FCBBF2000782, FCBBF2000808, FCBBF2000815, FCBBF2000825, FCBBF2000940, FCBBF2000951, FCBBF2001001, FCBBF2001088, FCBBF2001105, FCBBF2001116, FCBBF2001188, FCBBF2001238, FCBBF2001243, FCBBF2001309, FCBBF2001400, FCBBF2001427, FCBBF2001480, FCBBF2001529, FCBBF2001672, FCBBF2001675, FCBBF2001718, FCBBF2001720, FCBBF2001817, FCBBF2001868, FCBBF2002044, FCBBF2002111, FCBBF2002281, FCBBF2002370, FCBBF2002541, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003636, FCBBF2003823, FCBBF2003895, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005439, FCBBF2005538, FCBBF2005637, FCBBF2005645, FCBBF2005658, FCBBF2005733, FCBBF2006131, FCBBF2006380, FCBBF2006418, FCBBF2006452, FCBBF2006634, FCBBF2007012, FCBBF2007186, FCBBF2007265, FCBBF2007302, FCBBF2007610, FCBBF2007633, FCBBF3000102, FCBBF3000110, FCBBF3000120, FCBBF3000227, FCBBF3000229, FCBBF3000233, FCBBF3000361, FCBBF3000367, FCBBF3000462, FCBBF3000473, FCBBF3000501, FCBBF3000518, FCBBF3000536, FCBBF3000550, FCBBF3000847, FCBBF3001018, FCBBF3001020, FCBBF3001235, FCBBF3001281, FCBBF3001302, FCBBF3001377, FCBBF3001594, FCBBF3001890, FCBBF3001912, FCBBF3001914, FCBBF3001918, FCBBF3001924, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004261, FCBBF3004390, FCBBF3004409, FCBBF3004502, FCBBF3004715, FCBBF3004842, FCBBF3004847, FCBBF3004955, FCBBF3005160, FCBBF3005218, FCBBF3005320, FCBBF3005444, FCBBF3005497, FCBBF3005698, FCBBF3005729, FCBBF3006171, FCBBF3006249, FCBBF3006288, FCBBF3006399, FCBBF3006628, FCBBF3007077, FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007829, FCBBF3007855, FCBBF3008073, FCBBF3008159, FCBBF3008251, FCBBF3008311, FCBBF3008362, FCBBF3008382, FCBBF3008475, FCBBF3008556, FCBBF3008644, FCBBF3008748, FCBBF3008870, FCBBF3008938, FCBBF3008944, FCBBF3009101, FCBBF3009152, FCBBF3009256, FCBBF3009317, FCBBF3009428, FCBBF3009464, FCBBF3009479, FCBBF3009703, FCBBF3009717, FCBBF3009888, FCBBF3009949, FCBBF3009978, FCBBF3010008, FCBBF3010124, FCBBF3010130, FCBBF3010149, FCBBF3010211, FCBBF3010361, FCBBF3010544, FCBBF3010586, FCBBF3010601, FCBBF3010665, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012288, FCBBF3012332, FCBBF3012347, FCBBF3012443, FCBBF3012588, FCBBF3012842, FCBBF3012901, FCBBF3013041, FCBBF3013058, FCBBF3013205, FCBBF3013266, FCBBF3013307, FCBBF3013341, FCBBF3013589, FCBBF3013623, FCBBF3013800, FCBBF3013846, FCBBF3014229, FCBBF3014355, FCBBF3014567, FCBBF3014770, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016987, FCBBF3017024, FCBBF3017059, FCBBF3017071, FCBBF3017123, FCBBF3017233, FCBBF3017255, FCBBF3017288, FCBBF3017535, FCBBF3017681, FCBBF3017729, FCBBF3017873, FCBBF3017974, FCBBF3018067, FCBBF3018173, FCBBF3018591, FCBBF3018753, FCBBF3018949, FCBBF3019085, FCBBF3019437, FCBBF3019455, FCBBF3019564, FCBBF3019570, FCBBF3019663, FCBBF3019716, FCBBF3019784, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021940, FCBBF3021985, FCBBF3022005, FCBBF3022504, FCBBF3022566, FCBBF3022767, FCBBF3022894, FCBBF3023061, FCBBF3023078, FCBBF3023368, FCBBF3023372, FCBBF3023443, FCBBF3023599, FCBBF3023704, FCBBF3023887, FCBBF3023895, FCBBF3024096, FCBBF3024225, FCBBF3024364, FCBBF3024623, FCBBF3024911, FCBBF3025098, FCBBF3025252, FCBBF3025280, FCBBF3025285, FCBBF3025528, FCBBF3025568, FCBBF3025650, FCBBF3025674, FCBBF3025730, FCBBF3025737, FCBBF3026021, FCBBF3026048, FCBBF3026251, FCBBF3026308, FCBBF3026678, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028472, FCBBF3028528, FCBBF3028582, FCBBF3028593, FCBBF3028671, FCBBF3028794, FCBBF4000061, FCBBF4000076, FCBBF4000173, FCBBF4000192, FCBBF4000193, FCBBF4000268, FCBBF4000282, FCBBF4000399, FCBBF4000415, FCBBF4000500, FCBBF4000529, FCBBF5000041, FCBBF5000061, FCBBF5000261, FCBBF5000384, FCBBF5000483, FCBBF5000495, FEBRA1000005, FEBRA1000008, FEBRA1000030, FEBRA1000138, FEBRA1000148, FEBRA1000149, FEBRA1000183, FEBRA1000190, FEBRA2000021, FEBRA2000035, FEBRA2000053, FEBRA2000126, FEBRA2000220, FEBRA2000228, FEBRA2000253, FEBRA2000282, FEBRA2000286, FEBRA2000297, FEBRA2000311, FEBRA2000321, FEBRA2000333, FEBRA2000378, FEBRA2000391, FEBRA2000394, FEBRA2000397, FEBRA2000399, FEBRA2000409, FEBRA2000415, FEBRA2000436, FEBRA2000454, FEBRA2000468, FEBRA2000491, FEBRA2000504, FEBRA2000510, FEBRA2000532, FEBRA2000538, FEBRA2000575, FEBRA2000656, FEBRA2000659, FEBRA2000690, FEBRA2000727, FEBRA2000734, FEBRA2000762, FEBRA2000772, FEBRA2000790, FEBRA2000803, FEBRA2000805, FEBRA2000809, FEBRA2000856, FEBRA2000862, FEBRA2000901, FEBRA2000909, FEBRA2000917, FEBRA2000928, FEBRA2000936, FEBRA2000937, FEBRA2000959, FEBRA2001012, FEBRA2001020, FEBRA2001093, FEBRA2001124, FEBRA2001133, FEBRA2001161, FEBRA2001175, FEBRA2001217, FEBRA2001227, FEBRA2001267, FEBRA2001351, FEBRA2001438, FEBRA2001440, FEBRA2001487, FEBRA2001523, FEBRA2001561, FEBRA2001581, FEBRA2001584, FEBRA2001590, FEBRA2001591, FEBRA2001610, FEBRA2001620, FEBRA2001659, FEBRA2001698, FEBRA2001705, FEBRA2001706, FEBRA2001772, FEBRA2001790, FEBRA2001867, FEBRA2001914, FEBRA2001989, FEBRA2002009, FEBRA2002086, FEBRA2002109, FEBRA2002260, FEBRA2002399, FEBRA2002410, FEBRA2002429, FEBRA2002442, FEBRA2002508, FEBRA2002525, FEBRA2002552, FEBRA2002574, FEBRA2002601, FEBRA2002611, FEBRA2002628, FEBRA2002662, FEBRA2002682, FEBRA2002707, FEBRA2002727, FEBRA2002783, FEBRA2002818, FEBRA2002858, FEBRA2002882, FEBRA2002897, FEBRA2002908, FEBRA2002962, FEBRA2002986, FEBRA2003054, FEBRA2003115, FEBRA2003155, FEBRA2003163, FEBRA2003181, FEBRA2003198, FEBRA2003253, FEBRA2003275, FEBRA2003308, FEBRA2003327, FEBRA2003429, FEBRA2003454, FEBRA2003468, FEBRA2003520, FEBRA2003524, FEBRA2003675, FEBRA2003707, FEBRA2003711, FEBRA2003822, FEBRA2003897, FEBRA2003930, FEBRA2004023, FEBRA2004026, FEBRA2004029, FEBRA2004053, FEBRA2004056, FEBRA2004110, FEBRA2004191, FEBRA2004202, FEBRA2004219, FEBRA2004325, FEBRA2004329, FEBRA2004331, FEBRA2004443, FEBRA2004490, FEBRA2004538, FEBRA2004592, FEBRA2004651, FEBRA2004813, FEBRA2004840, FEBRA2004852, FEBRA2004867, FEBRA2004914, FEBRA2004933, FEBRA2004987, FEBRA2005014, FEBRA2005036, FEBRA2005216, FEBRA2005291, FEBRA2005377, FEBRA2005380, FEBRA2005427, FEBRA2005458, FEBRA2005701, FEBRA2005726, FEBRA2005778, FEBRA2005787, FEBRA2005995, FEBRA2005998, FEBRA2006055, FEBRA2006061, FEBRA2006150, FEBRA2006165, FEBRA2006315, FEBRA2006354, FEBRA2006370, FEBRA2006379, FEBRA2006409, FEBRA2006418, FEBRA2006427, FEBRA2006476, FEBRA2006491, FEBRA2006627, FEBRA2006664, FEBRA2006667, FEBRA2006726, FEBRA2006793, FEBRA2006817, FEBRA2006890, FEBRA2006942, FEBRA2006977, FEBRA2007176, FEBRA2007200, FEBRA2007247, FEBRA2007280, FEBRA2007414, FEBRA2007436, FEBRA2007449, FEBRA2007458, FEBRA2007551, FEBRA2007566, FEBRA2007620, FEBRA2007793, FEBRA2007818, FEBRA2007823, FEBRA2007931, FEBRA2007957, FEBRA2008009, FEBRA2008086, FEBRA2008135, FEBRA2008165, FEBRA2008172, FEBRA2008266, FEBRA2008282, FEBRA2008311, FEBRA2008341, FEBRA2008360, FEBRA2008468, FEBRA2008475, FEBRA2008481, FEBRA2008542, FEBRA2008660, FEBRA2008681, FEBRA2008692, FEBRA2008741, FEBRA2008755, FEBRA2008836, FEBRA2008866, FEBRA2008881, FEBRA2008983, FEBRA2009016, FEBRA2009029, FEBRA2009074, FEBRA2009117, FEBRA2009120, FEBRA2009162, FEBRA2009240, FEBRA2009265, FEBRA2009276, FEBRA2009327, FEBRA2009352, FEBRA2009362, FEBRA2009385, FEBRA2009416, FEBRA2009437, FEBRA2009478, FEBRA2009507, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012195, FEBRA2012397, FEBRA2012418, FEBRA2012625, FEBRA2013019, FEBRA2013069, FEBRA2013274, FEBRA2013465, FEBRA2013570, FEBRA2013905, FEBRA2013951, FEBRA2014010, FEBRA2014122, FEBRA2014198, FEBRA2014213, FEBRA2014443, FEBRA2014578, FEBRA2015076, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017098, FEBRA2017138, FEBRA2017154, FEBRA2017200, FEBRA2017223, FEBRA2017333, FEBRA2017502, FEBRA2017736, FEBRA2017780, FEBRA2017811, FEBRA2017850, FEBRA2018051, FEBRA2018407, FEBRA2019172, FEBRA2019298, FEBRA2019457, FEBRA2019519, FEBRA2019582, FEBRA2019637, FEBRA2019663, FEBRA2019690, FEBRA2019711, FEBRA2019867, FEBRA2020406, FEBRA2020566, FEBRA2020582, FEBRA2020668, FEBRA2020801, FEBRA2020845, FEBRA2020886, FEBRA2020977, FEBRA2021134, FEBRA2021339, FEBRA2021431, FEBRA2021497, FEBRA2021571, FEBRA2021892, FEBRA2021908, FEBRA2022013, FEBRA2022148, FEBRA2022204, FEBRA2022255, FEBRA2022322, FEBRA2022504, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023498, FEBRA2023721, FEBRA2023989, FEBRA2023990, FEBRA2024136, FEBRA2024150, FEBRA2024343, FEBRA2024570, FEBRA2024693, FEBRA2024727, FEBRA2024744, FEBRA2024797, FEBRA2025249, FEBRA2025463, FEBRA2025477, FEBRA2026474, FEBRA2026582, FEBRA2026629, FEBRA2026769, FEBRA2026879, FEBRA2026890, FEBRA2026977, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEHRT2000325, FEHRT2000364, FEKID1000018, FELIV1000101, FELIV1000164, FELNG2000241, HCASM1000021, HCASM1000050, HCASM1000061, HCASM1000130, HCASM2000202, HCASM2000266, HCASM2000307, HCASM2000363, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000131, HCHON1000142, HCHON1000176, HCHON2000028, HCHON2000038, HCHON2000087, HCHON2000100, HCHON2000156, HCHON2000199, HCHON2000212, HCHON2000271, HCHON2000315, HCHON2000322, HCHON2000344, HCHON2000364, HCHON2000373, HCHON2000395, HCHON2000417, HCHON2000418, HCHON2000503, HCHON2000660, HCHON2000663, HCHON2000673, HCHON2000699, HCHON2000705, HCHON2000738, HCHON2000751, HCHON2000817, HCHON2000818, HCHON2000832, HCHON2000851, HCHON2000898, HCHON2000935, HCHON2000942, HCHON2000956, HCHON2000994, HCHON2001084, HCHON2001099, HCHON2001110, HCHON2001116, HCHON2001214, HCHON2001233, HCHON2001269, HCHON2001450, HCHON2001453, HCHON2001519, HCHON2001523, HCHON2001535, HCHON2001546, HCHON2001548, HCHON2001577, HCHON2001598, HCHON2001604, HCHON2001607, HCHON2001646, HCHON2001712, HCHON2001768, HCHON2001801, HCHON2002123, HCHON2002194, HCHON2002241, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005987, HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HEART1000088, HEART1000099, HEART1000102, HEART1000118, HEART1000132, HEART1000139, HEART1000142, HEART1000151, HEART1000185, HEART2000024, HEART2000035, HEART2000099, HEART2000298, HEART2000309, HEART2000411, HEART2000418, HEART2000444, HEART2000448, HEART2000492, HEART2000541, HEART2000568, HEART2000611, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2004570, HEART2004764, HEART2004931, HEART2004940, HEART2004941, HEART2004980, HEART2005244, HEART2005581, HEART2005610, HEART2006131, HEART2006195, HEART2006310, HEART2006334, HEART2006557, HEART2006787, HEART2006789, HEART2006909, HEART2007031, HEART2007443, HEART2007696, HEART2007767, HEART2008108, HEART2008111, HEART2008257, HEART2008509, HEART2008994, HEART2009599, HEART2009680, HEART2010391, HEART2010492, HEART2010495, HELAC2000158, HELAC2000301, HHDPC1000001, HHDPC1000065, HHDPC1000118, HHDPC2000070, HHDPC2000102, HHDPC2000258, HHDPC2000315, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2006460, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HLUNG1000017, HLUNG1000024, HLUNG1000030, HLUNG1000034, HLUNG1000037, HLUNG1000053, HLUNG1000076, HLUNG1000084, HLUNG1000091, HLUNG1000099, HLUNG1000104, HLUNG1000105, HLUNG1000110, HLUNG1000151, HLUNG1000169, HLUNG2000004, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000255, HLUNG2000281, HLUNG2000315, HLUNG2000340, HLUNG2000362, HLUNG2000363, HLUNG2000431, HLUNG2000480, HLUNG2000489, HLUNG2000549, HLUNG2000557, HLUNG2000570, HLUNG2000751, HLUNG2000761, HLUNG2000777, HLUNG2000870, HLUNG2000884, HLUNG2000926, HLUNG2000950, HLUNG2000955, HLUNG2000970, HLUNG2001013, HLUNG2001126, HLUNG2001144, HLUNG2001214, HLUNG2001439, HLUNG2001459, HLUNG2001633, HLUNG2001677, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002562, HLUNG2002659, HLUNG2002707, HLUNG2002916, HLUNG2002942, HLUNG2002949, HLUNG2002958, HLUNG2002982, HLUNG2003003, HLUNG2003042, HLUNG2003049, HLUNG2003061, HLUNG2003162, HLUNG2003246, HLUNG2003306, HLUNG2003331, HLUNG2003335, HLUNG2003497, HLUNG2003691, HLUNG2003710, HLUNG2003714, HLUNG2003778, HLUNG2003833, HLUNG2003872, HLUNG2003918, HLUNG2003991, HLUNG2004014, HLUNG2004037, HLUNG2004154, HLUNG2004217, HLUNG2004273, HLUNG2004388, HLUNG2004416, HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006843, HLUNG2006852, HLUNG2006935, HLUNG2007041, HLUNG2007210, HLUNG2007219, HLUNG2007245, HLUNG2007433, HLUNG2007931, HLUNG2008066, HLUNG2008225, HLUNG2008235, HLUNG2008247, HLUNG2008333, HLUNG2008384, HLUNG2008396, HLUNG2008439, HLUNG2008479, HLUNG2008480, HLUNG2008521, HLUNG2008833, HLUNG2008875, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000148, HSYRA2000044, HSYRA2000135, HSYRA2000221, HSYRA2000237, HSYRA2000253, HSYRA2000332, HSYRA2000371, HSYRA2000424, HSYRA2000507, HSYRA2000510, HSYRA2000605, HSYRA2000760, HSYRA2000792, HSYRA2000832, HSYRA2000927, HSYRA2001103, HSYRA2001142, HSYRA2001153, HSYRA2001232, HSYRA2001255, HSYRA2001332, HSYRA2001353, HSYRA2001420, HSYRA2001484, HSYRA2001552, HSYRA2001580, HSYRA2001631, HSYRA2001638, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000161, IMR321000165, IMR321000207, IMR321000230, IMR321000253, IMR322000010, IMR322000107, IMR322000144, IMR322000152, IMR322000164, IMR322000178, IMR322000302, IMR322000374, IMR322000379, IMR322000383, IMR322000418, IMR322000604, IMR322000609, IMR322000656, IMR322000672, IMR322000791, IMR322000811, IMR322000838, IMR322000917, IMR322000919, IMR322000935, IMR322000953, IMR322000984, IMR322001018, IMR322001167, IMR322001185, IMR322001245, IMR322001452, IMR322001491, IMR322001665, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, JCMLC2000273, KIDNE1000008, KIDNE1000012, KIDNE1000015, KIDNE1000028, KIDNE1000036, KIDNE1000064, KIDNE1000121, KIDNE1000143, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000287, KIDNE2000315, KIDNE2000317, KIDNE2000328, KIDNE2000330, KIDNE20.00335, KIDNE2000375, KIDNE2000394, KIDNE2000403, KIDNE2000422, KIDNE2000493, KIDNE2000497, KIDNE2000510, KIDNE2000513, KIDNE2000517, KIDNE2000519, KIDNE2000555, KIDNE2000624, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2001117, KIDNE2001140, KIDNE2001269, KIDNE2001361, KIDNE2001373, KIDNE2001636, KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002198, KIDNE2002252, KIDNE2002262, KIDNE2002438, KIDNE2002483, KIDNE2002768, KIDNE2002795, KIDNE2002798, KIDNE2002839, KIDNE2002845, KIDNE2002872, KIDNE2002882, KIDNE2002883, KIDNE2002980, KIDNE2002991, KIDNE2003185, KIDNE2003188, KIDNE2003316, KIDNE2003335, KIDNE2003357, KIDNE2003373, KIDNE2003377, KIDNE2003507, KIDNE2003752, KIDNE2003837, KIDNE2003994, KIDNE2004034, KIDNE2004084, KIDNE2004095, KIDNE2004233, KIDNE2004262, KIDNE2004279, KIDNE2004294, KIDNE2004295, KIDNE2004305, KIDNE2004344, KIDNE2004394, KIDNE2004411, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006014, KIDNE2006030, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006652, KIDNE2006687, KIDNE2006733, KIDNE2006811, KIDNE2006820, KIDNE2007005, KIDNE2007040, KIDNE2007077, KIDNE2007186, KIDNE2007222, KIDNE2007328, KIDNE2007356, KIDNE2007422, KIDNE2007569, KIDNE2007810, KIDNE2007811, KIDNE2007910, KIDNE2007944, KIDNE2008022, KIDNE2008072, KIDNE2008116, KIDNE2008117, KIDNE2008218, KIDNE2008315, KIDNE2008378, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011782, KIDNE2011858, KIDNE2012025, KIDNE2012188, KIDNE2012316, KIDNE2012361, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014325, KIDNE2014415, KIDNE2014489, KIDNE2014496, KIDNE2014661, KIDNE2014717, KIDNE2014789, KIDNE2014808, KIDNE2014890, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015987, KIDNE2016000, KIDNE2016036, KIDNE2016096, KIDNE2016142, KIDNE2016188, KIDNE2016327, KIDNE2016388, KIDNE2016464, KIDNE2016539, KIDNE2016570, KIDNE2016918, KIDNE2017007, KIDNE2017052, KIDNE2017151, KIDNE2017315, KIDNE2017332, KIDNE2017343, KIDNE2017454, KIDNE2017603, KIDNE2017956, KIDNE2018166, KIDNE2018167, KIDNE2018254, KIDNE2018268, KIDNE2018269, KIDNE2018287, KIDNE2018294, KIDNE2018352, KIDNE2018462, KIDNE2018617, KIDNE2018678, KIDNE2018727, KIDNE2018863, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000082, LIVER1000099, LIVER1000111, LIVER1000126, LIVER1000139, LIVER1000155, LIVER1000165, LIVER1000175, LIVER1000224, LIVER1000278, LIVER1000303, LIVER1000397, LIVER1000421, LIVER1000433, LIVER1000479, LIVER1000482, LIVER1000542, LIVER2000003, LIVER2000158, LIVER2000216, LIVER2000237, LIVER2000247, LIVER2000360, LIVER2000416, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001329, LIVER2001389, LIVER2001461, LIVER2001539, LIVER2001608, LIVER2001835, LIVER2002638, LIVER2002644, LIVER2002660, LIVER2002702, LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006251, LIVER2006410, LIVER2006469, LIVER2006644, LIVER2006764, LIVER2007415, LIVER2007548, LIVER2007568, LIVER2007721, LIVER2008053, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000096, MAMGL1000132, MAMGL1000151, MAMGL1000178, MAMGL1000182, MAMGL1000184, MESAN1000032, MESAN1000035, MESAN1000079, MESAN1000121, MESAN1000147, MESAN1000180, MESAN2000023, MESAN2000026, MESAN2000067, MESAN2000092, MESAN2000149, MESAN2000167, MESAN2000264, MESAN2000291, MESAN2000337, MESAN2000434, MESAN2000457, MESAN2000462, MESAN2000620, MESAN2000711, MESAN2000815, MESAN2000894, MESAN2000909, MESAN2001627, MESAN2001770, MESAN2001979, MESAN2001980, MESAN2002113, MESAN2002147, MESAN2002424, MESAN2002687, MESAN2002709, MESAN2002790, MESAN2002844, MESAN2002940, MESAN2002978, MESAN2003035, MESAN2003037, MESAN2003039, MESAN2003058, MESAN2003101, MESAN2003190, MESAN2003322, MESAN2003444, MESAN2003490, MESAN2003529, MESAN2003646, MESAN2003662, MESAN2003851, MESAN2004138, MESAN2004288, MESAN2004575, MESAN2005284, MESAN2005303, MESAN2005371, MESAN2005633, MESAN2005724, MESAN2005808, MESAN2005811, MESAN2006022, MESAN2006043, MESAN2006328, MESAN2006580, MESAN2006599, MESAN2006743, MESAN2006953, MESAN2006971, MESAN2007032, MESAN2007440, MESAN2007597, MESAN2007755, MESAN2008222, MESAN2008415, MESAN2008460, MESAN2008536, MESAN2008679, MESAN2008821, MESAN2008926, MESAN2009019, MESAN2009081, MESAN2009105, MESAN2009311, MESAN2009418, MESAN2009503, MESAN2009522, MESAN2009580, MESAN2010031, MESAN2010114, MESAN2010312, MESAN2010321, MESAN2010681, MESAN2011545, MESAN2011597, MESAN2011627, MESAN2011632, MESAN2011977, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015391, MESAN2015401, MESAN2015501, MESAN2015708, MESAN2016159, MESAN2016304, MESAN2016409, MESAN2016552, MESAN2016965, MESAN2017152, MESAN2017417, MESAN2018209, MESAN2018576, MESAN2018670, MESTC1000147, MESTC2000150, MESTC2000153, MESTC2000170, N1ESE2000698, NB9N41000047, NB9N41000142, NB9N41000146, NB9N42000104, NB9N42000122, NB9N42000281, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2002352, NESOP2002418, NESOP2002487, NESOP2002738, NESOP2002780, NETRP1000034, NETRP1000082, NETRP2000086, NETRP2000182, NETRP2000439, NHNPC1000037, NHNPC1000084, NHNPC1000101, NHNPC1000124, NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000091, NOVAR1000102, NOVAR2000136, NOVAR2000352, NOVAR2000412, NOVAR2000710, NOVAR2000962, NOVAR2001108, NOVAR2001620, NOVAR2001783, NOVAR2001876, NOVAR2002039, NT2NE1000004, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000222, NT2NE2000284, NT2NE2000299, NT2NE2000353, NT2NE2000356, NT2NE2000369, NT2NE2000374, NT2NE2000386, NT2NE2000390, NT2NE2000455, NT2NE2000470, NT2NE2000488, NT2NE2000536, NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000809, NT2NE2000950, NT2NE2000951, NT2NE2000980, NT2NE2001005, NT2NE2001021, NT2NE2001049, NT2NE2001142, NT2NE2001180, NT2NE2001181, NT2NE2001188, NT2NE2001191, NT2NE2001247, NT2NE2001252, NT2NE2001326, NT2NE2001372, NT2NE2001428, NT2NE2001432, NT2NE2001524, NT2NE2001530, NT2NE2001598, NT2NE2001617, NT2NE2001623, NT2NE2001626, NT2NE2001634, NT2NE2001648, NT2NE2001660, NT2NE2001666, NT2NE2001677, NT2NE2001698, NT2NE2001723, NT2NE2001725, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003252, NT2NE2003280, NT2NE2003308, NT2NE2003309, NT2NE2003315, NT2NE2003393, NT2NE2003408, NT2NE2003457, NT2NE2003540, NT2NE2003571, NT2NE2003705, NT2NE2003752, NT2NE2003887, NT2NE2004255, NT2NE2004378, NT2NE2004490, NT2NE2004606, NT2NE2004716, NT2NE2004740, NT2NE2004787, NT2NE2004916, NT2NE2005035, NT2NE2005223, NT2NE2005276, NT2NE2005323, NT2NE2005358, NT2NE2005371, NT2NE2005395, NT2NE2005441, NT2NE2005517, NT2NE2005537, NT2NE2005555, NT2NE2005588, NT2NE2005676, NT2NE2005720, NT2NE2005784, NT2NE2005804, NT2NE2005821, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006478, NT2NE2006531, NT2NE2006784, NT2NE2006813, NT2NE2006894, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008867, NT2NE2008961, NT2NE2008997, NT2NE2009138, NT2NE2009295, NT2NE2009426, NT2NE2009608, NT2NE2009691, NT2NE2010105, NT2NE2010150, NT2NE2010400, NT2NE2010632, NT2NE2010633, NT2NE2010654, NT2NE2010688, NT2NE2010781, NT2NE2010854, NT2NE2011033, NT2NE2011036, NT2NE2011107, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012243, NT2NE2012361, NT2NE2012448, NT2NE2012457, NT2NE2012493, NT2NE2012533, NT2NE2012603, NT2NE2012747, NT2NE2012790, NT2NE2012847, NT2NE2013019, NT2NE2013081, NT2NE2013189, NT2NE2013217, NT2NE2013547, NT2NE2014013, NT2NE2014028, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015480, NT2NE2015511, NT2NE2015565, NT2NE2015626, NT2NE2015747, NT2NE2015860, NT2NE2015885, NT2NE2015904, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017982, NT2NE2018165, NT2NE2018176, NT2NE2018273, NT2NE2018376, NT2NE2018472, NT2NE2018490, NT2NE2018594, NT2NE2018739, NT2NE2018916, NT2NE2019056, NT2NE2019076, NT2NE2019099, NT2RI1000016, NT2RI1000035, NT2RI1000048, NT2RI1000164, NT2RI1000172, NT2RI2000004, NT2RI2000007, NT2RI2000117, NT2RI2000128, NT2RI2000173, NT2RI2000187, NT2RI2000270, NT2RI2000276, NT2RI2000313, NT2RI2000341, NT2RI2000348, NT2RI2000421, NT2RI2000480, NT2RI2000575, NT2RI2000588, NT2RI2000597, NT2RI2000669, NT2RI2000688, NT2RI2000689, NT2RI2000704, NT2RI2000738, NT2RI2000775, NT2RI2000822, NT2RI2000841, NT2RI2000865, NT2RI2000932, NT2RI2000974, NT2RI2000987, NT2RI2001010, NT2RI2001017, NT2RI2001030, NT2RI2001083, NT2RI2001167, NT2RI2001178, NT2RI2001235, NT2RI2001244, NT2RI2001307, NT2RI2001409, NT2RI2001410, NT2RI2001425, NT2RI2001449, NT2RI2001474, NT2RI2001519, NT2RI2001595, NT2RI2001624, NT2RI2001657, NT2RI2001726, NT2RI2001782, NT2RI2001836, NT2RI2001846, NT2RI2001910, NT2RI2001952, NT2RI2002022, NT2RI2002041, NT2RI2002117, NT2RI2002120, NT2RI2002153, NT2RI2002179, NT2RI2002252, NT2RI2002260, NT2RI2002270, NT2RI2002334, NT2RI2002359, NT2RI2002447, NT2RI2002481, NT2RI2002507, NT2RI2002517, NT2RI2002530, NT2RI2002549, NT2RI2002564, NT2RI2002592, NT2RI2002602, NT2RI2002729, NT2RI2002822, NT2RI2002852, NT2RI2002865, NT2RI2002923, NT2RI2002926, NT2RI2002970, NT2RI2003019, NT2RI2003067, NT2RI2003184, NT2RI2003360, NT2RI2003383, NT2RI2003392, NT2RI2003399, NT2RI2003419, NT2RI2003487, NT2RI2003667, NT2RI2003751, NT2RI2003884, NT2RI2003973, NT2RI2003993, NT2RI2004078, NT2RI2004079, NT2RI2004093, NT2RI2004136, NT2RI2004157, NT2RI2004188, NT2RI2004190, NT2RI2004209, NT2RI2004230, NT2RI2004312, NT2RI2004381, NT2RI2004442, NT2RI2004468, NT2RI2004606, NT2RI2004618, NT2RI2004783, NT2RI2004818, NT2RI2004840, NT2RI2004884, NT2RI2004916, NT2RI2004962, NT2RI2004984, NT2RI2004985, NT2RI2005048, NT2RI2005061, NT2RI2005069, NT2RI2005087, NT2RI2005096, NT2RI2005130, NT2RI2005315, NT2RI2005358, NT2RI2005368, NT2RI2005382, NT2RI2005520, NT2RI2005579, NT2RI2005621, NT2RI2005628, NT2RI2005647, NT2RI2005670, NT2RI2005710, NT2RI2005772, NT2RI2005814, NT2RI2005818, NT2RI2005966, NT2RI2006081, NT2RI2006126, NT2RI2006127, NT2RI2006183, NT2RI2006257, NT2RI2006294, NT2RI2006345, NT2RI2006403, NT2RI2006445, NT2RI2006487, NT2RI2006506, NT2RI2006553, NT2RI2006565, NT2RI2006659, NT2RI2006662, NT2RI2006667, NT2RI2006679, NT2RI2006686, NT2RI2006703, NT2RI2006716, NT2RI2006735, NT2RI2006762, NT2RI2006788, NT2RI2006838, NT2RI2006853, NT2RI2006855, NT2RI2006973, NT2RI2007046, NT2RI2007048, NT2RI2007116, NT2RI2007133, NT2RI2007214, NT2RI2007277, NT2RI2007303, NT2RI2007334, NT2RI2007386, NT2RI2007406, NT2RI2007439, NT2RI2007469, NT2RI2007507, NT2RI2007729, NT2RI2007751, NT2RI2007754, NT2RI2007827, NT2RI2007853, NT2RI2007877, NT2RI2007891, NT2RI2007956, NT2RI2007965, NT2RI2008007, NT2RI2008050, NT2RI2008204, NT2RI2008221, NT2RI2008233, NT2RI2008455, NT2RI2008502, NT2RI2008598, NT2RI2008622, NT2RI2008656, NT2RI2008801, NT2RI2008841, NT2RI2008952, NT2RI2009005, NT2RI2009101, NT2RI2009103, NT2RI2009144, NT2RI2009151, NT2RI2009215, NT2RI2009259, NT2RI2009289, NT2RI2009301, NT2RI2009517, NT2RI2009585, NT2RI2009595, NT2RI2009855, NT2RI2010283, NT2RI2010508, NT2RI2011422, NT2RI2011450, NT2RI2011683, NT2RI2011803, NT2RI2012990, NT2RI2013345, NT2RI2013357, NT2RI2013373, NT2RI2014247, NT2RI2014551, NT2RI2014683, NT2RI2014733, NT2RI2015239, NT2RI2016128, NT2RI2016134, NT2RI2017529, NT2RI2018311, NT2RI2018363, NT2RI2018448, NT2RI2018883, NT2RI2018950, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001458, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003409, NT2RI3003738, NT2RI3003925, NT2RI3004133, NT2RI3004381, NT2RI3005202, NT2RI3005403, NT2RI3005416, NT2RI3005861, NT2RI3005923, NT2RI3005928, NT2RI3006132, NT2RI3006171, NT2RI3006340, NT2RI3006376, NT2RI3006666, NT2RI3006796, NT2RI3007065, NT2RI3007095, NT2RI3007158, NT2RI3007167, NT2RI3007443, NT2RI3007684, NT2RI3007757, NT2RI3007878, NT2RI3007978, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000033, NT2RP6000043, NT2RP6000060, NT2RP6000077, NT2RP6000100, NT2RP6000119, NT2RP6000127, NT2RP6000140, NT2RP6000150, NT2RP7000041, NT2RP7000109, NT2RP7000112, NT2RP7000259, NT2RP7000311, NT2RP7000359, NT2RP7000364, NT2RP7000380, NT2RP7000391, NT2RP7000425, NT2RP7000926, NT2RP7001074, NT2RP7001080, NT2RP7001156, NT2RP7001306, NT2RP7001319, NT2RP7001335, NT2RP7001532, NT2RP7001591, NT2RP7001602, NT2RP7001678, NT2RP7001856, NT2RP7002028, NT2RP7002064, NT2RP7002151, NT2RP7002163, NT2RP7002243, NT2RP7002282, NT2RP7002376, NT2RP7002450, NT2RP7002478, NT2RP7002554, NT2RP7002603, NT2RP7002650, NT2RP7002779, NT2RP7002841, NT2RP7002978, NT2RP7002982, NT2RP7003025, NT2RP7003050, NT2RP7003084, NT2RP7003091, NT2RP7003107, NT2RP7003134, NT2RP7003148, NT2RP7003261, NT2RP7003439, NT2RP7003629, NT2RP7003680, NT2RP7004027, NT2RP7004080, NT2RP7004204, NT2RP7004233, NT2RP7004260, NT2RP7004348, NT2RP7004358, NT2RP7004396, NT2RP7004541, NT2RP7004559, NT2RP7004641, NT2RP7004656, NT2RP7004687, NT2RP7004722, NT2RP7004766, NT2RP7004961, NT2RP7004964, NT2RP7004975, NT2RP7005118, NT2RP7005205, NT2RP7005323, NT2RP7005468, NT2RP7005629, NT2RP7005631, NT2RP7005669, NT2RP7005846, NT2RP7006033, NT2RP7006075, NT2RP7006141, NT2RP7006160, NT2RP7006296, NT2RP7006304, NT2RP7006374, NT2RP7006395, NT2RP7006408, NT2RP7006457, NT2RP7006539, NT2RP7006547, NT2RP7006601, NT2RP7006621, NT2RP7006636, NT2RP7006853, NT2RP7006886, NT2RP7006887, NT2RP7006980, NT2RP7006986, NT2RP7006995, NT2RP7007114, NT2RP7007221, NT2RP7007226, NT2RP7007269, NT2RP7007310, NT2RP7007359, NT2RP7007381, NT2RP7007387, NT2RP7007406, NT2RP7007480, NT2RP7007504, NT2RP7007580, NT2RP7007594, NT2RP7007617, NT2RP7007643, NT2RP7007766, NT2RP7007925, NT2RP7007930, NT2RP7008013, NT2RP7008015, NT2RP7008133, NT2RP7008137, NT2RP7008142, NT2RP7008144, NT2RP7008315, NT2RP7008360, NT2RP7008406, NT2RP7008435, NT2RP7008454, NT2RP7008487, NT2RP7008544, NT2RP7008550, NT2RP7008557, NT2RP7008623, NT2RP7008657, NT2RP7008720, NT2RP7008855, NT2RP7008863, NT2RP7009019, NT2RP7009030, NT2RP7009147, NT2RP7009168, NT2RP7009215, NT2RP7009225, NT2RP7009236, NT2RP7009253, NT2RP7009267, NT2RP7009322, NT2RP7009363, NT2RP7009370, NT2RP7009397, NT2RP7009429, NT2RP7009439, NT2RP7009466, NT2RP7009502, NT2RP7009507, NT2RP7009577, NT2RP7009743, NT2RP7009867, NT2RP7010128, NT2RP7010235, NT2RP7010275, NT2RP7010521, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015512, NT2RP7015676, NT2RP7015789, NT2RP7015996, NT2RP7016508, NT2RP7016622, NT2RP7016911, NT2RP7017139, NT2RP7017284, NT2RP7017365, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019682, NT2RP7019835, NT2RP7020112, NT2RP7020123, NT2RP7020343, NT2RP7020379, NT2RP8000137, NT2RP8000296, NT2RP8000435, NT2RP8000483, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8008057, NT2RP8009248, NTONG1000006, NTONG1000047, NTONG1000052, NTONG1000075, NTONG1000087, NTONG1000123, NTONG1000130, NTONG1000161, NTONG1000172, NTONG1000213, NTONG1000214, NTONG1000247, NTONG1000257, NTONG2000040, NTONG2000231, NTONG2000363, NTONG2000413, NTONG2000492, NTONG2000499, NTONG2000531, NTONG2000583, NTONG2000800, NTONG2000858, NTONG2000878, NTONG2000966, NTONG2000977, NTONG2000985, NTONG2001014, NTONG2001079, NTONG2001137, NTONG2001157, NTONG2001173, NTONG2001220, NTONG2001222, NTONG2001362, NTONG2001532, NTONG2001567, NTONG2001587, NTONG2001619, NTONG2001642, NTONG2002278, NTONG2002582, NTONG2002807, NTONG2002948, NTONG2002970, NTONG2002985, NTONG2003158, NTONG2003210, NTONG2003316, NTONG2003409, NTONG2003515, NTONG2003805, NTONG2003839, NTONG2003852, NTONG2003897, NTONG2003928, NTONG2004095, NTONG2004308, NTONG2004521, NTONG2004614, NTONG2004690, NTONG2004806, NTONG2004829, NTONG2004918, NTONG2004991, NTONG2005062, NTONG2005086, NTONG2005137, NTONG2005153, NTONG2005265, NTONG2005277, NTONG2005363, NTONG2005373, NTONG2005480, NTONG2005497, NTONG2005577, NTONG2005657, NTONG2005822, NTONG2005897, NTONG2006099, NTONG2006187, NTONG2006301, NTONG2006324, NTONG2006354, NTONG2006440, NTONG2006501, NTONG2006646, NTONG2006709, NTONG2007020, NTONG2007028, NTONG2007034, NTONG2007249, NTONG2007517, NTONG2007522, NTONG2007693, NTONG2008088, NTONG2008093, NTONG2008143, NTONG2008365, NTONG2008483, NTONG2008522, NTONG2008672, NTONG2008862, NTONG2008939, NTONG2008944, NTONG2009060, NTONG2009068, NTONG2009229, NTONG2009233, OCBBF1000016, OCBBF1000030, OCBBF1000042, OCBBF1000054, OCBBF1000067, OCBBF1000080, OCBBF1000085, OCBBF1000091, OCBBF1000104, OCBBF1000118, OCBBF1000119, OCBBF1000122, OCBBF1000130, OCBBF1000138, OCBBF1000145, OCBBF1000153, OCBBF1000185, OCBBF1000192, OCBBF1000315, OCBBF2000013, OCBBF2000287, OCBBF2000452, OCBBF2000703, OCBBF2000719, OCBBF2000904, OCBBF2000998, OCBBF2001075, OCBBF2001101, OCBBF2001124, OCBBF2001176, OCBBF2001252, OCBBF2001323, OCBBF2001389, OCBBF2001402, OCBBF2001408, OCBBF2001416, OCBBF2001473, OCBBF2001494, OCBBF2001527, OCBBF2001528, OCBBF2001586, OCBBF2001681, OCBBF2001706, OCBBF2001749, OCBBF2001910, OCBBF2001961, OCBBF2001983, OCBBF2002083, OCBBF2002124, OCBBF2002161, OCBBF2002357, OCBBF2002615, OCBBF2002663, OCBBF2002805, OCBBF2002865, OCBBF2002920, OCBBF2002980, OCBBF2003028, OCBBF2003091, OCBBF2003327, OCBBF2003543, OCBBF2003593, OCBBF2003744, OCBBF2004038, OCBBF2004147, OCBBF2004168, OCBBF2004259, OCBBF2004385, OCBBF2004418, OCBBF2004478, OCBBF2004482, OCBBF2004533, OCBBF2004567, OCBBF2004612, OCBBF2004647, OCBBF2004669, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005546, OCBBF2005843, OCBBF2005901, OCBBF2006005, OCBBF2006030, OCBBF2006058, OCBBF2006148, OCBBF2006154, OCBBF2006214, OCBBF2006241, OCBBF2006388, OCBBF2006438, OCBBF2006567, OCBBF2006624, OCBBF2006639, OCBBF2006660, OCBBF2006849, OCBBF2006859, OCBBF2006972, OCBBF2006987, OCBBF2007039, OCBBF2007051, OCBBF2007068, OCBBF2007121, OCBBF2007184, OCBBF2007194, OCBBF2007196, OCBBF2007238, OCBBF2007414, OCBBF2007428, OCBBF2007478, OCBBF2007485, OCBBF2007520, OCBBF2007555, OCBBF2007610, OCBBF2007756, OCBBF2007892, OCBBF2007931, OCBBF2007946, OCBBF2008041, OCBBF2008116, OCBBF2008138, OCBBF2008283, OCBBF2008330, OCBBF2008366, OCBBF2008466, OCBBF2008483, OCBBF2008520, OCBBF2008569, OCBBF2008586, OCBBF2008640, OCBBF2008691, OCBBF2008724, OCBBF2008760, OCBBF2008768, OCBBF2008770, OCBBF2008790, OCBBF2008814, OCBBF2008822, OCBBF2009115, OCBBF2009391, OCBBF2009424, OCBBF2009536, OCBBF2009571, OCBBF2009583, OCBBF2009603, OCBBF2009788, OCBBF2009920, OCBBF2009926, OCBBF2010040, OCBBF2010140, OCBBF2010281, OCBBF2010404, OCBBF2010416, OCBBF2010420, OCBBF2010604, OCBBF2010709, OCBBF2010792, OCBBF2010830, OCBBF2010841, OCBBF2010843, OCBBF2010931, OCBBF2010945, OCBBF2010978, OCBBF2010989, OCBBF2011137, OCBBF2011160, OCBBF2011177, OCBBF2011228, OCBBF2011283, OCBBF2011311, OCBBF2011625, OCBBF2011685, OCBBF2011706, OCBBF2011722, OCBBF2011767, OCBBF2011855, OCBBF2011872, OCBBF2011960, OCBBF2011981, OCBBF2012001, OCBBF2012028, OCBBF2012095, OCBBF2012139, OCBBF2012191, OCBBF2012262, OCBBF2012320, OCBBF2012436, OCBBF2012755, OCBBF2012936, OCBBF2012990, OCBBF2013049, OCBBF2013091, OCBBF2013123, OCBBF2013127, OCBBF2013208, OCBBF2013319, OCBBF2013507, OCBBF2013721, OCBBF2013789, OCBBF2013883, OCBBF2013926, OCBBF2014052, OCBBF2014322, OCBBF2014386, OCBBF2014541, OCBBF2014707, OCBBF2014828, OCBBF2014873, OCBBF2014880, OCBBF2014928, OCBBF2015031, OCBBF2015081, OCBBF2015232, OCBBF2015285, OCBBF2015334, OCBBF2015335, OCBBF2015503, OCBBF2015506, OCBBF2015599, OCBBF2015645, OCBBF2015659, OCBBF2015797, OCBBF2015931, OCBBF2015980, OCBBF2016038, OCBBF2016405, OCBBF2016467, OCBBF2016590, OCBBF2016591, OCBBF2016612, OCBBF2016690, OCBBF2016841, OCBBF2016928, OCBBF2017069, OCBBF2017306, OCBBF2017325, OCBBF2017489, OCBBF2017516, OCBBF2017665, OCBBF2017815, OCBBF2017888, OCBBF2017920, OCBBF2018014, OCBBF2018167, OCBBF2018206, OCBBF2018229, OCBBF2018362, OCBBF2018380, OCBBF2018563, OCBBF2018663, OCBBF2018687, OCBBF2018828, OCBBF2018873, OCBBF2018934, OCBBF2019108, OCBBF2019195, OCBBF2019327, OCBBF2019684, OCBBF2019761, OCBBF2019823, OCBBF2019950, OCBBF2020048, OCBBF2020318, OCBBF2020453, OCBBF2020801, OCBBF2020825, OCBBF2020838, OCBBF2021020, OCBBF2021214, OCBBF2021323, OCBBF2021518, OCBBF2021788, OCBBF2021833, OCBBF2022573, OCBBF2022574, OCBBF2023598, OCBBF2023643, OCBBF2024463, OCBBF2024589, OCBBF2024719, OCBBF2024779, OCBBF2024781, OCBBF2025028, OCBBF2025115, OCBBF2025198, OCBBF2025451, OCBBF2025458, OCBBF2025631, OCBBF2025730, OCBBF2026025, OCBBF2026035, OCBBF2026144, OCBBF2026645, OCBBF2026690, OCBBF2026981, OCBBF2027148, OCBBF2027354, OCBBF2027423, OCBBF2027478, OCBBF2027481, OCBBF2027511, OCBBF2027728, OCBBF2027782, OCBBF2027868, OCBBF2028055, OCBBF2028173, OCBBF2028421, OCBBF2029471, OCBBF2029901, OCBBF2030116, OCBBF2030354, OCBBF2030517, OCBBF2030574, OCBBF2030708, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033413, OCBBF2033869, OCBBF2033948, OCBBF2034333, OCBBF2034529, OCBBF2034637, OCBBF2034906, OCBBF2035110, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009279, PANCR1000012, PANCR1000021, PANCR1000081, PANCR1000086, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000180, PEBLM2000030, PEBLM2000112, PEBLM2000126, PEBLM2000170, PEBLM2000180, PEBLM2000213, PEBLM2000248, PEBLM2000270, PEBLM2000308, PEBLM2000321, PEBLM2000333, PEBLM2000338, PEBLM2000395, PEBLM2000479, PEBLM2000502, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003935, PEBLM2004015, PEBLM2004216, PEBLM2004290, PEBLM2004497, PEBLM2004733, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006366, PEBLM2006709, PEBLM2006912, PEBLM2007112, PEBLM2007140, PEBLM2007188, PEBLM2007296, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005305, PERIC2005347, PERIC2005370, PERIC2005936, PERIC2006035, PERIC2006121, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000067, PLACE5000068, PLACE5000070, PLACE5000080, PLACE5000105, PLACE5000115, PLACE5000116, PLACE5000129, PLACE5000139, PLACE5000153, PLACE5000171, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000296, PLACE6000314, PLACE6000325, PLACE6000348, PLACE6000371, PLACE6000424, PLACE6000426, PLACE6000429, PLACE6000450, PLACE6000463, PLACE6000523, PLACE6000550, PLACE6000552, PLACE6000555, PLACE6000630, PLACE6000953, PLACE6001118, PLACE6001153, PLACE6001294, PLACE6001536, PLACE6001557, PLACE6001712, PLACE6001740, PLACE6001886, PLACE6001923, PLACE6001925, PLACE6001933, PLACE6002011, PLACE6002016, PLACE6002056, PLACE6002157, PLACE6002312, PLACE6002323, PLACE6002345, PLACE6002462, PLACE6002647, PLACE6002668, PLACE6002699, PLACE6002710, PLACE6002871, PLACE6002949, PLACE6002960, PLACE6003004, PLACE6003077, PLACE6003094, PLACE6003109, PLACE6003204, PLACE6003218, PLACE6003372, PLACE6003383, PLACE6003393, PLACE6003399, PLACE6003539, PLACE6003705, PLACE6003740, PLACE6003745, PLACE6003771, PLACE6003796, PLACE6003839, PLACE6003850, PLACE6004101, PLACE6004219, PLACE6004336, PLACE6004368, PLACE6004380, PLACE6004396, PLACE6004454, PLACE6004464, PLACE6004491, PLACE6004663, PLACE6004687, PLACE6004738, PLACE6004931, PLACE6004966, PLACE6004993, PLACE6005007, PLACE6005029, PLACE6005108, PLACE6005234, PLACE6005283, PLACE6005294, PLACE6005328, PLACE6005351, PLACE6005423, PLACE6005487, PLACE6005535, PLACE6005546, PLACE6005559, PLACE6005579, PLACE6005786, PLACE6006042, PLACE6006077, PLACE6006158, PLACE6006186, PLACE6006217, PLACE6006222, PLACE6006266, PLACE6006287, PLACE6006394, PLACE6006418, PLACE6006474, PLACE6006566, PLACE6006697, PLACE6006822, PLACE6006871, PLACE6006905, PLACE6006988, PLACE6007050, PLACE6007239, PLACE6007242, PLACE6007309, PLACE6007380, PLACE6007433, PLACE6007482, PLACE6007687, PLACE6007787, PLACE6007798, PLACE6007807, PLACE6007925, PLACE6008036, PLACE6008094, PLACE6008126, PLACE6008285, PLACE6008453, PLACE6008640, PLACE6008750, PLACE6008768, PLACE6008793, PLACE6008824, PLACE6008989, PLACE6009006, PLACE6009008, PLACE6009228, PLACE6009237, PLACE6009419, PLACE6009430, PLACE6009482, PLACE6009524, PLACE6009527, PLACE6009560, PLACE6009590, PLACE6009821, PLACE6009835, PLACE6009853, PLACE6010077, PLACE6010463, PLACE6010485, PLACE6010568, PLACE6010701, PLACE6010704, PLACE6010732, PLACE6010765, PLACE6010848, PLACE6010925, PLACE6010991, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012108, PLACE6012168, PLACE6012297, PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013883, PLACE6013884, PLACE6013885, PLACE6014043, PLACE6014516, PLACE6014882, PLACE6015211, PLACE6015322, PLACE6015460, PLACE6015513, PLACE6015591, PLACE6015731, PLACE6015939, PLACE6016030, PLACE6016093, PLACE6016105, PLACE6016160, PLACE6016210, PLACE6016254, PLACE6016383, PLACE6016984, PLACE6017002, PLACE6017431, PLACE6017564, PLACE6017578, PLACE6017626, PLACE6017701, PLACE6017714, PLACE6017788, PLACE6017791, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6018938, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008431, PLACE7008623, PLACE7008766, PLACE7009563, PROST1000033, PROST1000039, PROST1000065, PROST1000083, PROST1000085, PROST1000110, PROST1000120, PROST1000123, PROST1000136, PROST1000144, PROST1000145, PROST1000152, PROST1000167, PROST1000184, PROST1000217, PROST1000246, PROST1000262, PROST1000298, PROST1000322, PROST1000334, PROST1000343, PROST1000362, PROST1000377, PROST1000383, PROST1000412, PROST1000419, PROST1000451, PROST1000480, PROST1000485, PROST1000493, PROST1000512, PROST1000527, PROST1000528, PROST1000536, PROST1000559, PROST1000575, PROST2000036, PROST2000053, PROST2000067, PROST2000079, PROST2000105, PROST2000143, PROST2000162, PROST2000165, PROST2000203, PROST2000206, PROST2000212, PROST2000241, PROST2000267, PROST2000273, PROST2000274, PROST2000277, PROST2000325, PROST2000337, PROST2000432, PROST2000452, PROST2000463, PROST2000470, PROST2000505, PROST2000572, PROST2000717, PROST2000760, PROST2000835, PROST2000893, PROST2000961, PROST2001116, PROST2001138, PROST2001213, PROST2001414, PROST2001415, PROST2001465, PROST2001540, PROST2001552, PROST2001599, PROST2001611, PROST2001636, PROST2001739, PROST2001796, PROST2001805, PROST2001823, PROST2001899, PROST2001998, PROST2002078, PROST2002101, PROST2002212, PROST2002338, PROST2002488, PROST2002489, PROST2002527, PROST2002591, PROST2002602, PROST2002633, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003302, PROST2003303, PROST2003324, PROST2003340, PROST2003428, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004258, PROST2004332, PROST2004481, PROST2004526, PROST2004727, PROST2004742, PROST2004817, PROST2005039, PROST2005076, PROST2005121, PROST2005131, PROST2005228, PROST2005272, PROST2005426, PROST2005630, PROST2005788, PROST2005880, PROST2005886, PROST2005904, PROST2005919, PROST2005943, PROST2006008, PROST2006020, PROST2006196, PROST2006201, PROST2006282, PROST2006343, PROST2006450, PROST2006491, PROST2006510, PROST2006536, PROST2006579, PROST2006688, PROST2006766, PROST2006782, PROST2006933, PROST2007122, PROST2007200, PROST2007237, PROST2007289, PROST2007317, PROST2007328, PROST2007382, PROST2007389, PROST2007416, PROST2007528, PROST2007612, PROST2007818, PROST2007871, PROST2007950, PROST2007956, PROST2007974, PROST2007988, PROST2008079, PROST2008088, PROST2008243, PROST2008245, PROST2008268, PROST2008447, PROST2008468, PROST2008472, PROST2008516, PROST2008724, PROST2008835, PROST2008993, PROST2009022, PROST2009208, PROST2009222, PROST2009254, PROST2009273, PROST2009320, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010400, PROST2010545, PROST2010579, PROST2010606, PROST2010782, PROST2010885, PROST2011012, PROST2011038, PROST2011297, PROST2011439, PROST2011482, PROST2011631, PROST2012005, PROST2012088, PROST2012140, PROST2012157, PROST2012190, PROST2012249, PROST2012353, PROST2012400, PROST2012448, PROST2012504, PROST2012542, PROST2012550, PROST2012740, PROST2012780, PROST2012888, PROST2012890, PROST2013032, PROST2013053, PROST2013260, PROST2013327, PROST2013531, PROST2013605, PROST2013627, PROST2013873, PROST2013880, PROST2014422, PROST2014585, PROST2014601, PROST2014659, PROST2014916, PROST2014925, PROST2015124, PROST2015137, PROST2015198, PROST2015243, PROST2015246, PROST2015251, PROST2015287, PROST2015457, PROST2015464, PROST2015537, PROST2015545, PROST2015551, PROST2015636, PROST2015710, PROST2015756, PROST2015877, PROST2015924, PROST2015932, PROST2016195, PROST2016246, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016980, PROST2017098, PROST2017367, PROST2017413, PROST2017441, PROST2017578, PROST2017612, PROST2017617, PROST2017700, PROST2017701, PROST2017729, PROST2017836, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019398, PROST2019487, PROST2019781, PUAEN1000039, PUAEN1000081, PUAEN1000085, PUAEN1000087, PUAEN1000121, PUAEN1000161, PUAEN1000175, PUAEN1000239, PUAEN1000275, PUAEN1000276, PUAEN1000288, PUAEN2000080, PUAEN2000175, PUAEN2000247, PUAEN2000312, PUAEN2000394, PUAEN2000497, PUAEN2000535, PUAEN2000594, PUAEN2000684, PUAEN2000942, PUAEN2001260, PUAEN2001345, PUAEN2001526, PUAEN2001882, PUAEN2002473, PUAEN2002552, PUAEN2002568, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007350, PUAEN2007785, PUAEN2008123, PUAEN2008228, PUAEN2008314, PUAEN2008815, PUAEN2008939, PUAEN2008980, PUAEN2008985, PUAEN2009348, PUAEN2009534, PUAEN2009655, PUAEN2009795, PUAEN2009852, PUAEN2010346, RECTM1000051, RECTM1000141, RECTM2000220, RECTM2000349, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000065, SALGL1000107, SALGL1000171, SKMUS1000014, SKMUS1000030, SKMUS1000064, SKMUS1000067, SKMUS1000102, SKMUS1000104, SKMUS1000118, SKMUS1000124, SKMUS1000129, SKMUS1000138, SKMUS1000176, SKMUS2000061, SKMUS2000078, SKMUS2000137, SKMUS2000198, SKMUS2000287, SKMUS2000317, SKMUS2000330, SKMUS2000339, SKMUS2000365, SKMUS2000390, SKMUS2000416, SKMUS2000458, SKMUS2000467, SKMUS2000468, SKMUS2000515, SKMUS2000679, SKMUS2000690, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000894, SKMUS2000898, SKMUS2000933, SKMUS2000945, SKMUS2000961, SKMUS2000973, SKMUS2000980, SKMUS2001014, SKMUS2001147, SKMUS2001157, SKMUS2001164, SKMUS2001199, SKMUS2001215, SKMUS2001247, SKMUS2001271, SKMUS2001287, SKMUS2001323, SKMUS2001364, SKMUS2001398, SKMUS2001492, SKMUS2001580, SKMUS2001585, SKMUS2001608, SKMUS2001631, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000110, SKNMC1000123, SKNMC1000124, SKNMC1000137, SKNMC1000159, SKNMC1000229, SKNMC2000256, SKNMC2000356, SKNMC2000374, SKNMC2000583, SKNMC2000593, SKNMC2000612, SKNMC2000622, SKNMC2001324, SKNMC2001555, SKNMC2001596, SKNMC2001623, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000086, SKNSH1000174, SKNSH1000301, SKNSH1000308, SKNSH1000416, SKNSH2000051, SKNSH2000516, SKNSH2000550, SKNSH2000716, SKNSH2000971, SKNSH2001222, SKNSH2001875, SKNSH2001931, SKNSH2002054, SKNSH2002866, SKNSH2003174, SKNSH2003422, SKNSH2003466, SKNSH2003483, SKNSH2004039, SKNSH2005120, SKNSH2005194, SKNSH2005240, SKNSH2005792, SKNSH2005816, SKNSH2005981, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009357, SKNSH2009435, SKNSH2009991, SKNSH2010015, SMINT1000012, SMINT1000016, SMINT1000039, SMINT1000042, SMINT1000048, SMINT1000051, SMINT1000054, SMINT1000057, SMINT1000071, SMINT1000075, SMINT1000078, SMINT1000091, SMINT1000103, SMINT1000118, SMINT1000137, SMINT2000007, SMINT2000032, SMINT2000040, SMINT2000060, SMINT2000084, SMINT2000145, SMINT2000159, SMINT2000176, SMINT2000185, SMINT2000227, SMINT2000229, SMINT2000239, SMINT2000267, SMINT2000396, SMINT2000400, SMINT2000518, SMINT2000529, SMINT2000530, SMINT2000537, SMINT2000538, SMINT2000541, SMINT2000545, SMINT2000558, SMINT2000562, SMINT2000568, SMINT2000609, SMINT2000747, SMINT2000811, SMINT2000824, SMINT2000984, SMINT2001114, SMINT2001158, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001950, SMINT2002126, SMINT2002202, SMINT2002210, SMINT2002224, SMINT2002281, SMINT2002311, SMINT2002314, SMINT2002328, SMINT2002414, SMINT2002457, SMINT2002620, SMINT2002743, SMINT2002778, SMINT2002880, SMINT2002882, SMINT2002884, SMINT2002899, SMINT2002974, SMINT2002976, SMINT2003074, SMINT2003128, SMINT2003317, SMINT2003386, SMINT2003423, SMINT2003505, SMINT2003551, SMINT2003569, SMINT2003641, SMINT2003644, SMINT2003866, SMINT2003905, SMINT2004023, SMINT2004037, SMINT2004047, SMINT2004086, SMINT2004144, SMINT2004280, SMINT2004339, SMINT2004473, SMINT2004547, SMINT2004729, SMINT2004781, SMINT2004891, SMINT2004909, SMINT2004992, SMINT2005075, SMINT2005161, SMINT2005174, SMINT2005213, SMINT2005330, SMINT2005387, SMINT2005405, SMINT2005621, SMINT2005623, SMINT2005624, SMINT2005688, SMINT2005800, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007219, SMINT2007294, SMINT2007365, SMINT2007391, SMINT2007433, SMINT2007508, SMINT2007526, SMINT2007579, SMINT2007790, SMINT2007796, SMINT2007867, SMINT2007932, SMINT2008042, SMINT2008054, SMINT2008277, SMINT2008329, SMINT2008455, SMINT2008491, SMINT2008531, SMINT2008545, SMINT2008558, SMINT2008590, SMINT2008625, SMINT2008690, SMINT2008715, SMINT2008720, SMINT2008817, SMINT2008844, SMINT2008869, SMINT2008917, SMINT2009119, SMINT2009121, SMINT2009212, SMINT2009216, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010144, SMINT2010200, SMINT2010278, SMINT2010324, SMINT2010326, SMINT2010369, SMINT2010426, SMINT2010500, SMINT2010533, SMINT2010629, SMINT2010672, SMINT2010753, SMINT2010853, SMINT2010897, SMINT2010901, SMINT2010959, SMINT2010997, SMINT2011033, SMINT2011066, SMINT2011273, SMINT2011311, SMINT2011406, SMINT2011509, SMINT2011567, SMINT2011588, SMINT2011888, SMINT2011958, SMINT2012040, SMINT2012122, SMINT2012179, SMINT2012285, SMINT2012734, SMINT2012793, SMINT2013031, SMINT2013129, SMINT2013170, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000134, SPLEN1000139, SPLEN1000143, SPLEN1000144, SPLEN1000145, SPLEN1000156, SPLEN1000163, SPLEN1000178, SPLEN2000020, SPLEN2000047, SPLEN2000064, SPLEN2000072, SPLEN2000126, SPLEN2000134, SPLEN2000143, SPLEN2000189, SPLEN2000197, SPLEN2000205, SPLEN2000210, SPLEN2000222, SPLEN2000242, SPLEN2000243, SPLEN2000247, SPLEN2000258, SPLEN2000270, SPLEN2000307, SPLEN2000310, SPLEN2000350, SPLEN2000357, SPLEN2000364, SPLEN2000402, SPLEN2000414, SPLEN2000420, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695, SPLEN2000839, SPLEN2000882, SPLEN2001135, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001626, SPLEN2001650, SPLEN2001689, SPLEN2001710, SPLEN2001781, SPLEN2001881, SPLEN2001912, SPLEN2001945, SPLEN2002053, SPLEN2002133, SPLEN2002147, SPLEN2002166, SPLEN2002175, SPLEN2002223, SPLEN2002272, SPLEN2002314, SPLEN2002343, SPLEN2002354, SPLEN2002385, SPLEN2002419, SPLEN2002451, SPLEN2002462, SPLEN2002477, SPLEN2002662, SPLEN2002695, SPLEN2002707, SPLEN2002744, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004220, SPLEN2004346, SPLEN2004368, SPLEN2004611, SPLEN2004880, SPLEN2004984, SPLEN2005009, SPLEN2005227, SPLEN2005272, SPLEN2005342, SPLEN2005416, SPLEN2005450, SPLEN2005474, SPLEN2005560, SPLEN2005727, SPLEN2005790, SPLEN2005818, SPLEN2005838, SPLEN2005869, SPLEN2005927, SPLEN2005939, SPLEN2006122, SPLEN2006232, SPLEN2006268, SPLEN2006283, SPLEN2006325, SPLEN2006389, SPLEN2006425, SPLEN2006701, SPLEN2006785, SPLEN2006875, SPLEN2007182, SPLEN2007276, SPLEN2007314, SPLEN2007326, SPLEN2007350, SPLEN2007388, SPLEN2007619, SPLEN2007653, SPLEN2007689, SPLEN2007695, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008591, SPLEN2008786, SPLEN2009081, SPLEN2009088, SPLEN2009163, SPLEN2009315, SPLEN2009340, SPLEN2009512, SPLEN2009541, SPLEN2009548, SPLEN2009555, SPLEN2009581, SPLEN2009733, SPLEN2009803, SPLEN2009884, SPLEN2009918, SPLEN2009970, SPLEN2010004, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010862, SPLEN2010978, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011678, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012523, SPLEN2012524, SPLEN2012525, SPLEN2012606, SPLEN2012611, SPLEN2012619, SPLEN2012725, SPLEN2012743, SPLEN2012756, SPLEN2012800, SPLEN2012846, SPLEN2012889, SPLEN2012961, SPLEN2013108, SPLEN2013503, SPLEN2013644, SPLEN2013648, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015815, SPLEN2015891, SPLEN2015899, SPLEN2016045, SPLEN2016053, SPLEN2016069, SPLEN2016098, SPLEN2016135, SPLEN2016241, SPLEN2016250, SPLEN2016304, SPLEN2016356, SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016781, SPLEN2016840, SPLEN2016863, SPLEN2016922, SPLEN2016932, SPLEN2016973, SPLEN2017043, SPLEN2017104, SPLEN2017147, SPLEN2017187, SPLEN2017189, SPLEN2017212, SPLEN2017426, SPLEN2017452, SPLEN2017538, SPLEN2017613, SPLEN2017620, SPLEN2017643, SPLEN2017687, SPLEN2017740, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019311, SPLEN2019323, SPLEN2019349, SPLEN2019375, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019985, SPLEN2020034, SPLEN2020153, SPLEN2020154, SPLEN2020166, SPLEN2020183, SPLEN2020223, SPLEN2020359, SPLEN2020427, SPLEN2020467, SPLEN2020512, SPLEN2020608, SPLEN2020777, SPLEN2020805, SPLEN2021057, SPLEN2021122, SPLEN2021157, SPLEN2021194, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025219, SPLEN2025491, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2030250, SPLEN2030335, SPLEN2030371, SPLEN2030397, SPLEN2030479, SPLEN2030495, SPLEN2030562, SPLEN2030847, SPLEN2031004, SPLEN2031125, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036953, SPLEN2037077, SPLEN2037319, SPLEN2037444, SPLEN2037580, SPLEN2037630, SPLEN2037678, SPLEN2037722, SPLEN2037891, SPLEN2038055, SPLEN2038180, SPLEN2038407, SPLEN2038473, SPLEN2038834, SPLEN2039311, SPLEN2039672, SPLEN2039697, SPLEN2039845, SPLEN2039936, SPLEN2040222, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042598, SPLEN2042714, SPLEN2042920, STOMA1000013, STOMA1000047, STOMA1000052, STOMA1000074, STOMA1000091, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000121, STOMA2000148, STOMA2000168, STOMA2000183, STOMA2000188, STOMA2000257, STOMA2000279, STOMA2000383, STOMA2000386, STOMA2000478, STOMA2000529, STOMA2000539, STOMA2000567, STOMA2000640, STOMA2000678, STOMA2000686, STOMA2000888, STOMA2001025, STOMA2001389, STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, SYNOV1000045, SYNOV1000049, SYNOV1000118, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000824, SYNOV2000923, SYNOV2001014, SYNOV2001035, SYNOV2001088, SYNOV2001111, SYNOV2001212, SYNOV2001251, SYNOV2001300, SYNOV2001356, SYNOV2001374, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV3000184, SYNOV3000231, SYNOV3000302, SYNOV3000345, SYNOV4000249, SYNOV4000472, SYNOV4000598, SYNOV4000706, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007711, SYNOV4008336, SYNOV4009129, SYNOV4009139, SYNOV4009298, T1ESE2000116, T1ESE2000609, T1ESE2000904, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000613, TCERX2000998, TCOLN1000037, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002489, TESOP2002539, TESOP2002545, TESOP2002804, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000051, TESTI1000055, TESTI1000064, TESTI1000085, TESTI1000088, TESTI1000093, TESTI1000094, TESTI1000101, TESTI1000109, TESTI1000125, TESTI1000127, TESTI1000128, TESTI1000131, TESTI1000138, TESTI1000142, TESTI1000157, TESTI1000163, TESTI1000169, TESTI1000179, TESTI1000183, TESTI1000191, TESTI1000257, TESTI1000266, TESTI1000319, TESTI1000348, TESTI1000390, TESTI1000391, TESTI1000459, TESTI1000491, TESTI1000545, TESTI2000006, TESTI2000018, TESTI2000044, TESTI2000117, TESTI2000120, TESTI2000147, TESTI2000154, TESTI2000172, TESTI2000177, TESTI2000179, TESTI2000207, TESTI2000208, TESTI2000221, TESTI2000238, TESTI2000253, TESTI2000278, TESTI2000349, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000600, TESTI2000616, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000951, TESTI2000981, TESTI2001008, TESTI2001049, TESTI2001082, TESTI2001120, TESTI2001134, TESTI2001141, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001512, TESTI2001518, TESTI2001556, TESTI2001593, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001869, TESTI2001879, TESTI2001894, TESTI2001898, TESTI2001915, TESTI2001950, TESTI2001959, TESTI2001968, TESTI2001991, TESTI2002002, TESTI2002012, TESTI2002036, TESTI2002048, TESTI2002057, TESTI2002081, TESTI2002090, TESTI2002105, TESTI2002149, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003020, TESTI2003031, TESTI2003037, TESTI2003044, TESTI2003059, TESTI2003061, TESTI2003074, TESTI2003089, TESTI2003104, TESTI2003109, TESTI2003117, TESTI2003130, TESTI2003131, TESTI2003139, TESTI2003152, TESTI2003181, TESTI2003193, TESTI2003196, TESTI2003228, TESTI2003236, TESTI2003255, TESTI2003258, TESTI2003277, TESTI2003299, TESTI2003311, TESTI2003321, TESTI2003327, TESTI2003330, TESTI2003347, TESTI2003354, TESTI2003356, TESTI2003372, TESTI2003376, TESTI2003413, TESTI2003418, TESTI2003419, TESTI2003432, TESTI2003454, TESTI2003475, TESTI2003476, TESTI2003498, TESTI2003512, TESTI2003539, TESTI2003545, TESTI2003551, TESTI2003574, TESTI2003579, TESTI2003580, TESTI2003589, TESTI2003596, TESTI2003598, TESTI2003615, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004085, TESTI2004089, TESTI2004111, TESTI2004119, TESTI2004163, TESTI2004169, TESTI2004207, TESTI2004215, TESTI2004227, TESTI2004229, TESTI2004243, TESTI2004255, TESTI2004287, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004783, TESTI2004791, TESTI2004793, TESTI2004860, TESTI2004906, TESTI2004920, TESTI2004936, TESTI2004941, TESTI2004971, TESTI2004982, TESTI2004993, TESTI2004994, TESTI2004999, TESTI2005017, TESTI2005025, TESTI2005040, TESTI2005060, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005775, TESTI2005827, TESTI2005835, TESTI2005860, TESTI2005892, TESTI2005911, TESTI2005933, TESTI2005937, TESTI2005948, TESTI2005979, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006109, TESTI2006125, TESTI2006179, TESTI2006209, TESTI2006212, TESTI2006218, TESTI2006238, TESTI2006255, TESTI2006258, TESTI2006278, TESTI2006333, TESTI2006360, TESTI2006409, TESTI2006425, TESTI2006437, TESTI2006457, TESTI2006460, TESTI2006465, TESTI2006483, TESTI2006499, TESTI2006523, TESTI2006543, TESTI2006565, TESTI2006572, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007183, TESTI2007220, TESTI2007234, TESTI2007320, TESTI2007346, TESTI2007358, TESTI2007402, TESTI2007405, TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009325, TESTI2009390, TESTI2009400, TESTI2009412, TESTI2009423, TESTI2009447, TESTI2009462, TESTI2009477, TESTI2009497, TESTI2009502, TESTI2009511, TESTI2009544, TESTI2009545, TESTI2009551, TESTI2009577, TESTI2009585, TESTI2009588, TESTI2009727, TESTI2009739, TESTI2009812, TESTI2009835, TESTI2009853, TESTI2009935, TESTI2009977, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011575, TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012152, TESTI2012155, TESTI2012171, TESTI2012207, TESTI2012231, TESTI2012244, TESTI2012308, TESTI2012356, TESTI2012387, TESTI2012394, TESTI2012444, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013257, TESTI2013264, TESTI2013268, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014843, TESTI2014979, TESTI2014988, TESTI2015042, TESTI2015105, TESTI2015180, TESTI2015249, TESTI2015327, TESTI2015331, TESTI2015335, TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016599, TESTI2016629, TESTI2016663, TESTI2016667, TESTI2016688, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017069, TESTI2017089, TESTI2017102, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018941, TESTI2018957, TESTI2018974, TESTI2018975, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020871, TESTI2020905, TESTI2020906, TESTI2020946, TESTI2020956, TESTI2020981, TESTI2020999, TESTI2021003, TESTI2021057, TESTI2021077, TESTI2021112, TESTI2021114, TESTI2021116, TESTI2021118, TESTI2021122, TESTI2021138, TESTI2021297, TESTI2021315, TESTI2021358, TESTI2021447, TESTI2021463, TESTI2021599, TESTI2021637, TESTI2021667, TESTI2021736, TESTI2021785, TESTI2021819, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025448, TESTI2025454, TESTI2025486, TESTI2025546, TESTI2025582, TESTI2025609, TESTI2025656, TESTI2025757, TESTI2025791, TESTI2025872, TESTI2025879, TESTI2025911, TESTI2025920, TESTI2025924, TESTI2026014, TESTI2026024, TESTI2026064, TESTI2026077, TESTI2026104, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026534, TESTI2026537, TESTI2026589, TESTI2026597, TESTI2026605, TESTI2026674, TESTI2026794, TESTI2026824, TESTI2026854, TESTI2026925, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028384, TESTI2028426, TESTI2028488, TESTI2028523, TESTI2028583, TESTI2028613, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034931, TESTI2034953, TESTI2034997, TESTI2035078, TESTI2035084, TESTI2035107, TESTI2035121, TESTI2035124, TESTI2035183, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036684, TESTI2036691, TESTI2036736, TESTI2036822, TESTI2036833, TESTI2036913, TESTI2036922, TESTI2036951, TESTI2036965, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000370, TESTI4000394, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001473, TESTI4001517, TESTI4001527, TESTI4001569, TESTI4001604, TESTI4001665, TESTI4001679, TESTI4001795, TESTI4001923, TESTI4001925, TESTI4001984, TESTI4002003, TESTI4002141, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4003179, TESTI4003279, TESTI4003319, TESTI4003404, TESTI4003565, TESTI4003574, TESTI4003579, TESTI4003733, TESTI4003796, TESTI4003944, TESTI4004003, TESTI4004031, TESTI4004200, TESTI4004210, TESTI4004539, TESTI4004653, TESTI4004695, TESTI4004722, TESTI4004887, TESTI4004917, TESTI4005015, TESTI4005039, TESTI4005158, TESTI4005317, TESTI4005322, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009286, TESTI4009306, TESTI4009374, TESTI4009406, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013602, TESTI4013667, TESTI4013685, TESTI4013735, TESTI4013742, TESTI4013774, TESTI4013830, TESTI4013852, TESTI4013924, TESTI4013960, TESTI4013962, TESTI4014175, TESTI4014265, TESTI4014276, TESTI4014392, TESTI4014415, TESTI4014445, TESTI4014661, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037228, TESTI4037244, TESTI4037727, TESTI4037949, TESTI4038047, TESTI4038156, TESTI4038223, TESTI4038258, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044770, TESTI4045168, TESTI4045312, TESTI4045330, TESTI4045470, TESTI4045701, TESTI4045908, TESTI4045955, TESTI4045982, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000017, THYMU1000025, THYMU1000029, THYMU1000033, THYMU1000060, THYMU1000074, THYMU1000098, THYMU1000105, THYMU1000136, THYMU1000142, THYMU1000158, THYMU1000176, THYMU1000179, THYMU1000191, THYMU1000217, THYMU1000240, THYMU1000242, THYMU1000291, THYMU1000316, THYMU1000318, THYMU1000329, THYMU1000336, THYMU1000366, THYMU1000382, THYMU1000393, THYMU1000394, THYMU1000399, THYMU1000426, THYMU1000428, THYMU1000440, THYMU1000459, THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000440, THYMU2000492, THYMU2000519, THYMU2000570, THYMU2000602, THYMU2000660, THYMU2000671, THYMU2000672, THYMU2000702, THYMU2000767, THYMU2000775, THYMU2000800, THYMU2000879, THYMU2000932, THYMU2000950, THYMU2000971, THYMU2000982, THYMU2000990, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002815, THYMU2002852, THYMU2002910, THYMU2002950, THYMU2003069, THYMU2003107, THYMU2003124, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003470, THYMU2003479, THYMU2003499, THYMU2003632, THYMU2003650, THYMU2003700, THYMU2003760, THYMU2003803, THYMU2003855, THYMU2003907, THYMU2003932, THYMU2003981, THYMU2004003, THYMU2004008, THYMU2004042, THYMU2004108, THYMU2004111, THYMU2004123, THYMU2004139, THYMU2004169, THYMU2004320, THYMU2004336, THYMU2004356, THYMU2004452, THYMU2004512, THYMU2004635, THYMU2004639, THYMU2004668, THYMU2004677, THYMU2004688, THYMU2004693, THYMU2004835, THYMU2004843, THYMU2004906, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005546, THYMU2005576, THYMU2005748, THYMU2005759, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006913, THYMU2006946, THYMU2006965, THYMU2007025, THYMU2007060, THYMU2007095, THYMU2007112, THYMU2007146, THYMU2007179, THYMU2007258, THYMU2007307, THYMU2007308, THYMU2007339, THYMU2007350, THYMU2007384, THYMU2007493, THYMU2007521, THYMU2007528, THYMU2007530, THYMU2007561, THYMU2007571, THYMU2007635, THYMU2007658, THYMU2007667, THYMU2007725, THYMU2007802, THYMU2007824, THYMU2007854, THYMU2007952, THYMU2007969, THYMU2008035, THYMU2008049, THYMU2008072, THYMU2008111, THYMU2008145, THYMU2008149, THYMU2008185, THYMU2008191, THYMU2008207, THYMU2008226, THYMU2008282, THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009596, THYMU2009792, THYMU2009835, THYMU2009906, THYMU2009948, THYMU2010030, THYMU2010046, THYMU2010059, THYMU2010082, THYMU2010094, THYMU2010161, THYMU2010192, THYMU2010519, THYMU2010637, THYMU2010671, THYMU2010699, THYMU2010705, THYMU2010831, THYMU2010848, THYMU2011053, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2012002, THYMU2012073, THYMU2012104, THYMU2012113, THYMU2012273, THYMU2012401, THYMU2012492, THYMU2012507, THYMU2012565, THYMU2012574, THYMU2012625, THYMU2012631, THYMU2012665, THYMU2012701, THYMU2012807, THYMU2012882, THYMU2012891, THYMU2012902, THYMU2012919, THYMU2013023, THYMU2013047, THYMU2013051, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014167, THYMU2014183, THYMU2014204, THYMU2014297, THYMU2014327, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015479, THYMU2015762, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017675, THYMU2017707, THYMU2017831, THYMU2017844, THYMU2017948, THYMU2018028, THYMU2018071, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019054, THYMU2019197, THYMU2019210, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023576, THYMU2023711, THYMU2023900, THYMU2023943, THYMU2024121, THYMU2024185, THYMU2024207, THYMU2024616, THYMU2024684, THYMU2024748, THYMU2025042, THYMU2025189, THYMU2025319, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029676, THYMU2029688, THYMU2029788, THYMU2030068, THYMU2030226, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2035064, THYMU2035078, THYMU2035101, THYMU2035319, THYMU2035321, THYMU2035388, THYMU2035400, THYMU2035554, THYMU2035710, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037233, THYMU2037234, THYMU2037348, THYMU2037406, THYMU2037965, THYMU2038058, THYMU2038189, THYMU2038199, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039788, THYMU2039989, THYMU2040029, THYMU2040110, THYMU2040114, THYMU2040140, THYMU2040412, THYMU2040824, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000390, THYMU3000490, THYMU3000504, THYMU3000542, THYMU3000655, THYMU3000826, THYMU3000841, THYMU3001077, THYMU3001082, THYMU3001133, THYMU3001151, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003350, THYMU3003403, THYMU3003565, THYMU3003763, THYMU3003865, THYMU3003958, THYMU3003965, THYMU3004157, THYMU3004628, THYMU3004632, THYMU3004866, THYMU3005050, THYMU3005378, THYMU3005407, THYMU3005415, THYMU3005427, THYMU3005482, THYMU3005491, THYMU3005696, THYMU3006118, THYMU3006132, THYMU3006168, THYMU3006172, THYMU3006371, THYMU3006640, THYMU3006767, THYMU3006811, THYMU3006963, THYMU3007137, THYMU3007368, THYMU3007559, THYMU3007845, THYMU3008060, THYMU3008136, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019605, THYMU3019916, THYMU3020221, THYMU3020595, THYMU3020713, THYMU3020856, THYMU3020869, THYMU3020970, THYMU3021404, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3033402, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046360, THYMU3046856, THYMU3047115, THYMU3047144, THYMU3047156, THYMU3047366, THYMU3047513, THYMU3047542, THYMU3047760, THYMU3047891, TKIDN1000044, TKIDN1000062, TKIDN1000066, TKIDN1000171, TKIDN2000127, TKIDN2000240, TKIDN2000319, TKIDN2000464, TKIDN2000521, TKIDN2000701, TKIDN2001529, TKIDN2001662, TKIDN2002329, TKIDN2002424, TKIDN2002632, TKIDN2003044, TKIDN2003059, TKIDN2003062, TKIDN2003078, TKIDN2003366, TKIDN2003396, TKIDN2003413, TKIDN2003597, TKIDN2004185, TKIDN2004386, TKIDN2004458, TKIDN2004748, TKIDN2005721, TKIDN2005934, TKIDN2005947, TKIDN2005956, TKIDN2006525, TKIDN2006852, TKIDN2007667, TKIDN2008778, TKIDN2009092, TKIDN2009330, TKIDN2009481, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015161, TKIDN2015263, TKIDN2016309, TKIDN2016399, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004110, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000018, TRACH1000038, TRACH1000057, TRACH1000063, TRACH1000074, TRACH1000100, TRACH1000106, TRACH1000125, TRACH1000140, TRACH1000144, TRACH1000212, TRACH1000240, TRACH2000002, TRACH2000015, TRACH2000024, TRACH2000032, TRACH2000081, TRACH2000083, TRACH2000148, TRACH2000185, TRACH2000196, TRACH2000235, TRACH2000237, TRACH2000243, TRACH2000250, TRACH2000259, TRACH2000272, TRACH2000282, TRACH2000287, TRACH2000289, TRACH2000306, TRACH2000312, TRACH2000336, TRACH2000359, TRACH2000411, TRACH2000496, TRACH2000497, TRACH2000502, TRACH2000522, TRACH2000540, TRACH2000559, TRACH2000665, TRACH2000675, TRACH2000703, TRACH2000767, TRACH2000775, TRACH2000807, TRACH2000862, TRACH2000894, TRACH2000898, TRACH2000923, TRACH2000944, TRACH2000981, TRACH2000986, TRACH2001037, TRACH2001060, TRACH2001085, TRACH2001101, TRACH2001121, TRACH2001147, TRACH2001154, TRACH2001188, TRACH2001192, TRACH2001275, TRACH2001335, TRACH2001339, TRACH2001347, TRACH2001395, TRACH2001400, TRACH2001403, TRACH2001416, TRACH2001428, TRACH2001443, TRACH2001463, TRACH2001474, TRACH2001525, TRACH2001532, TRACH2001592, TRACH2001596, TRACH2001621, TRACH2001651, TRACH2001898, TRACH2001933, TRACH2001968, TRACH2001996, TRACH2002043, TRACH2002054, TRACH2002092, TRACH2002100, TRACH2002138, TRACH2002333, TRACH2002784, TRACH2002840, TRACH2002847, TRACH2002853, TRACH2002862, TRACH2002954, TRACH2002960, TRACH2002988, TRACH2003070, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004428, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005811, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007754, TRACH2007816, TRACH2007894, TRACH2007969, TRACH2007988, TRACH2008081, TRACH2008112, TRACH2008127, TRACH2008130, TRACH2008278, TRACH2008478, TRACH2008540, TRACH2008583, TRACH2008597, TRACH2008820, TRACH2008921, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010883, TRACH2010965, TRACH2010974, TRACH2011057, TRACH2011093, TRACH2011290, TRACH2011293, TRACH2011302, TRACH2011500, TRACH2011574, TRACH2011956, TRACH2012000, TRACH2012138, TRACH2012242, TRACH2012298, TRACH2012370, TRACH2012387, TRACH2012497, TRACH2012536, TRACH2012562, TRACH2012742, TRACH2012823, TRACH2012918, TRACH2013081, TRACH2013123, TRACH2013265, TRACH2013369, TRACH2013450, TRACH2013495, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2015180, TRACH2015243, TRACH2015316, TRACH2015381, TRACH2015486, TRACH2015654, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084, TRACH2018262, TRACH2018273, TRACH2018278, TRACH2018317, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025379, TRACH2025507, TRACH2025569, TRACH2025705, TRACH2025749, TRACH2025753, TRACH2025810, TRACH2025853, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002866, TRACH3002871, TRACH3002876, TRACH3002890, TRACH3003009, TRACH3003037, TRACH3003357, TRACH3003379, TRACH3003586, TRACH3003683, TRACH3003780, TRACH3003805, TRACH3003832, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005699, TRACH3005702, TRACH3005808, TRACH3005852, TRACH3005896, TRACH3006149, TRACH3006228, TRACH3006264, TRACH3006336, TRACH3006379, TRACH3006392, TRACH3006395, TRACH3006397, TRACH3006470, TRACH3006589, TRACH3006626, TRACH3006699, TRACH3006800, TRACH3006889, TRACH3006985, TRACH3007097, TRACH3007268, TRACH3007274, TRACH3007277, TRACH3007391, TRACH3007541, TRACH3007595, TRACH3007627, TRACH3007689, TRACH3007766, TRACH3007866, TRACH3008042, TRACH3008061, TRACH3008201, TRACH3008508, TRACH3008535, TRACH3008629, TRACH3008688, TRACH3008697, TRACH3008713, TRACH3008887, TRACH3008902, TRACH3008990, TRACH3009008, TRACH3009059, TRACH3009061, TRACH3009148, TRACH3009455, TRACH3009701, TRACH3009715, TRACH3009894, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036997, TRACH3037063, TRACH3037267, TRACH3037288, TRACH3037505, TRACH3037573, TRACH3037696, TRACH3037897, TRACH3038086, TSTOM1000135, TSTOM1000186, TSTOM2000139, TSTOM2000235, TSTOM2000315, TSTOM2000442, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000916, TUTER2001286, TUTER2001341, TUTER2001387, TUTER2001433, TUTER2001461, TUTER2002028, TUTER2002074, TUTER2002158, TUTER2002228, TUTER2002356, TUTER2002729, UMVEN1000143, UMVEN1000156, UMVEN1000186, UMVEN2000046, UMVEN2000069, UMVEN2000121, UMVEN2000354, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000183, UTERU1000187, UTERU1000192, UTERU1000249, UTERU1000337, UTERU1000339, UTERU2000047, UTERU2000099, UTERU2000154, UTERU2000197, UTERU2000218, UTERU2000238, UTERU2000243, UTERU2000260, UTERU2000300, UTERU2000329, UTERU2000332, UTERU2000338, UTERU2000349, UTERU2000377, UTERU2000393, UTERU2000418, UTERU2000465, UTERU2000485, UTERU2000517, UTERU2000524, UTERU2000537, UTERU2000539, UTERU2000541, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002198, UTERU2002294, UTERU2002332, UTERU2002473, UTERU2002547, UTERU2002693, UTERU2002733, UTERU2002736, UTERU2002737, UTERU2002841, UTERU2002964, UTERU2002993, UTERU2003035, UTERU2003126, UTERU2003135, UTERU2003321, UTERU2003399, UTERU2003411, UTERU2003577, UTERU2003926, UTERU2003973, UTERU2004015, UTERU2004037, UTERU2004039, UTERU2004061, UTERU2004073, UTERU2004163, UTERU2004299, UTERU2004461, UTERU2004520, UTERU2004564, UTERU2004688, UTERU2004698, UTERU2004807, UTERU2005004, UTERU2005050, UTERU2005069, UTERU2005074, UTERU2005179, UTERU2005292, UTERU2005346, UTERU2005450, UTERU2005533, UTERU2005601, UTERU2005664, UTERU2005822, UTERU2005903, UTERU2005905, UTERU2006103, UTERU2006137, UTERU2006182, UTERU2006400, UTERU2006412, UTERU2006429, UTERU2006486, UTERU2006524, UTERU2006547, UTERU2006568, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639, UTERU2007724, UTERU2007942, UTERU2008018, UTERU2008019, UTERU2008027, UTERU2008077, UTERU2008130, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008939, UTERU2008962, UTERU2009094, UTERU2009120, UTERU2009131, UTERU2009147, UTERU2009206, UTERU2009283, UTERU2009414, UTERU2009483, UTERU2009538, UTERU2009540, UTERU2009776, UTERU2009904, UTERU2009951, UTERU2009972, UTERU2010115, UTERU2010124, UTERU2010164, UTERU2010231, UTERU2010304, UTERU2010320, UTERU2010431, UTERU2010525, UTERU2010651, UTERU2010724, UTERU2011195, UTERU2011199, UTERU2011261, UTERU2011287, UTERU2011410, UTERU2011574, UTERU2011657, UTERU2011741, UTERU2011806, UTERU2011811, UTERU2011897, UTERU2011962, UTERU2011968, UTERU2012031, UTERU2012101, UTERU2012114, UTERU2012252, UTERU2012333, UTERU2012581, UTERU2012610, UTERU2012615, UTERU2012688, UTERU2012703, UTERU2012715, UTERU2012741, UTERU2012767, UTERU2012786, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001, UTERU2014223, UTERU2014398, UTERU2014464, UTERU2014548, UTERU2014631, UTERU2014668, UTERU2014678, UTERU2014728, UTERU2014998, UTERU2015062, UTERU2015087, UTERU2015108, UTERU2015190, UTERU2015198, UTERU2015202, UTERU2015405, UTERU2015640, UTERU2015653, UTERU2015830, UTERU2015880, UTERU2016147, UTERU2016157, UTERU2016426, UTERU2016464, UTERU2016669, UTERU2016757, UTERU2016761, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017762, UTERU2017810, UTERU2017988, UTERU2018127, UTERU2018180, UTERU2018333, UTERU2018364, UTERU2018514, UTERU2018522, UTERU2018523, UTERU2018544, UTERU2018609, UTERU2018674, UTERU2018784, UTERU2018789, UTERU2018811, UTERU2018867, UTERU2018881, UTERU2018955, UTERU2019038, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2022020, UTERU2022773, UTERU2022955, UTERU2022981, UTERU2023039, UTERU2023045, UTERU2023175, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2025025, UTERU2025041, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000402, UTERU3000645, UTERU3000665, UTERU3000738, UTERU3000828, UTERU3000844, UTERU3000899, UTERU3000959, UTERU3001029, UTERU3001053, UTERU3001059, UTERU3001240, UTERU3001394; UTERU3001542, UTERU3001558, UTERU3001571, UTERU3001585, UTERU3001632, UTERU3001652, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005264, UTERU3005422, UTERU3005585, UTERU3005907, UTERU3006008, UTERU3006228, UTERU3006538, UTERU3006640, UTERU3006687, UTERU3006720, UTERU3006798, UTERU3006884, UTERU3007021, UTERU3007104, UTERU3007108, UTERU3007134, UTERU3007640, UTERU3007731, UTERU3008280, UTERU3008463, UTERU3008660, UTERU3008671, UTERU3008722, UTERU3008917, UTERU3009259, UTERU3009490, UTERU3009690, UTERU3009775, UTERU3009871, UTERU3009979, UTERU3009987, UTERU3010029, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the BRAWH, BRCAN, BRCOC, BRHIP, BRSSN, BRSTN, and BRTHA libraries were analyzed for expression frequency. The analysis results are described below. Each is a relative value, where the expression level over all tissues is taken as 100. The larger this value, the greater the level of expression.

TABLE 28

| Clone Name | BRAWH | BRCAN | BRCOC | BRHIP | BRSSN | BRSTN | BRTHA |
|---|---|---|---|---|---|---|---|
| 3NB691000116 | 1.626 | 3.731 | 0 | 0 | 0 | 0 | 1.803 |
| 3NB691000173 | 0 | 0 | 0 | 0.806 | 0 | 2.847 | 0 |
| 3NB691000191 | 6.122 | 0 | 0 | 6.246 | 0 | 0 | 0 |
| 3NB692000281 | 0 | 1.010 | 0 | 0.225 | 0 | 0 | 0.244 |
| 3NB692000330 | 0 | 0 | 0 | 0 | 1.642 | 1.590 | 0.978 |
| 3NB692000374 | 2.169 | 0 | 0 | 3.319 | 8.073 | 0 | 2.405 |
| 3NB692000529 | 0 | 0 | 0 | 11.376 | 0 | 0 | 0 |
| 3NB692000912 | 0 | 1.078 | 0 | 0 | 1.749 | 0 | 0 |
| 3NB692001022 | 0 | 0 | 0 | 1.269 | 4.631 | 0 | 0 |
| 3NB692001288 | 0 | 13.749 | 10.615 | 0 | 5.576 | 0 | 1.661 |
| 3NB692001334 | 0.268 | 0.616 | 2.853 | 0 | 1.998 | 8.711 | 0.595 |
| 3NB692001433 | 0 | 0 | 0 | 0 | 0 | 0 | 3.815 |
| 3NB692001442 | 0 | 5.543 | 0 | 0 | 0 | 0 | 1.340 |
| 3NB692001528 | 2.352 | 0 | 0 | 0 | 0 | 0 | 0 |
| 3NB692001557 | 0 | 0 | 0 | 6.189 | 0 | 0 | 0 |
| 3NB692001719 | 0 | 0 | 24.400 | 7.027 | 0 | 0 | 0 |
| ADIPS2000425 | 1.553 | 0 | 0 | 1.584 | 0 | 0 | 0 |
| ADRGL1000002 | 1.647 | 0 | 0 | 6.720 | 0 | 0 | 5.478 |
| ADRGL1000018 | 9.672 | 11.095 | 0 | 14.801 | 0 | 0 | 0 |
| ADRGL1000033 | 0 | 0 | 13.526 | 0 | 0 | 0 | 0 |
| ADRGL1000065 | 0 | 0 | 0 | 5.753 | 0 | 0 | 6.254 |
| ADRGL1000144 | 0 | 0 | 0 | 3.410 | 0 | 12.051 | 0 |
| ADRGL2000006 | 4.158 | 0 | 0 | 3.182 | 0 | 0 | 2.306 |
| ADRGL2000042 | 2.540 | 0 | 0 | 0 | 18.905 | 0 | 0 |
| ADRGL2000097 | 0 | 0 | 0 | 0 | 0 | 8.016 | 0 |
| ADRGL2002013 | 17.992 | 0 | 0 | 0 | 0 | 0 | 0 |
| ADRGL2002191 | 0 | 0 | 0 | 13.043 | 0 | 0 | 0 |
| ADRGL2002477 | 3.402 | 3.903 | 0 | 3.471 | 0 | 12.268 | 3.773 |
| ADRGL2002679 | 12.443 | 0 | 0 | 0 | 0 | 0 | 0 |
| ADRGL2003785 | 0 | 0 | 0 | 14.971 | 0 | 0 | 0 |
| ADRGL2007906 | 18.024 | 20.676 | 0 | 0 | 0 | 0 | 0 |
| ADRGL2009273 | 2.778 | 0 | 0 | 7.086 | 5.170 | 0 | 4.621 |
| ADRGL2010152 | 0 | 0 | 0 | 0 | 21.560 | 0 | 0 |
| ASTRO1000009 | 2.611 | 0 | 0 | 0 | 0 | 0 | 0 |
| ASTRO2000014 | 1.232 | 0 | 0 | 0.419 | 0 | 1.481 | 0.911 |
| ASTRO2000046 | 5.305 | 0 | 0 | 11.240 | 0 | 0 | 4.525 |
| ASTRO2000372 | 0 | 0 | 0 | 22.748 | 0 | 0 | 0 |
| ASTRO2000381 | 0 | 0 | 0 | 6.407 | 0 | 0 | 0 |
| ASTRO2000480 | 0 | 0 | 0 | 2.888 | 0 | 0 | 0 |
| ASTRO2000482 | 22.398 | 0 | 0 | 0 | 0 | 0 | 0 |
| ASTRO2000662 | 0 | 18.504 | 0 | 0 | 0 | 0 | 0 |
| ASTRO2000914 | 1.947 | 0 | 0 | 0 | 0 | 0 | 0 |
| ASTRO2001029 | 0 | 2.877 | 0 | 0 | 0 | 0 | 4.172 |
| ASTRO2001076 | 0 | 0 | 5.035 | 2.900 | 0 | 0 | 1.576 |
| ASTRO2001107 | 14.609 | 0 | 0 | 14.905 | 0 | 0 | 16.201 |
| ASTRO2001823 | 0 | 0 | 0 | 1.481 | 0 | 0 | 0 |
| ASTRO2002035 | 0 | 0 | 0 | 22.147 | 0 | 0 | 0 |
| ASTRO2002202 | 2.304 | 0 | 0 | 0 | 0 | 0 | 0 |
| ASTRO2002733 | 0 | 0 | 0 | 15.696 | 0 | 0 | 0 |
| ASTRO2003024 | 0.948 | 4.349 | 3.358 | 0 | 0 | 0 | 2.102 |
| ASTRO2003316 | 2.950 | 0 | 0 | 0 | 0 | 0 | 0 |
| ASTRO2003461 | 5.957 | 0 | 0 | 0 | 0 | 0 | 0 |
| ASTRO2003574 | 0 | 0 | 0 | 15.221 | 0 | 0 | 33.089 |
| ASTRO2003740 | 0 | 39.838 | 0 | 0 | 0 | 0 | 0 |
| ASTRO2003840 | 0 | 0 | 0 | 0 | 0 | 0 | 6.869 |
| ASTRO2004628 | 26.702 | 0 | 0 | 0 | 0 | 0 | 0 |
| ASTRO2004974 | 1.943 | 0 | 0 | 1.982 | 7.232 | 0 | 0 |
| ASTRO2005360 | 5.966 | 0 | 0 | 0 | 0 | 0 | 6.616 |
| ASTRO2005423 | 6.886 | 0 | 0 | 17.565 | 0 | 0 | 11.455 |
| ASTRO2005863 | 7.603 | 0 | 0 | 0 | 0 | 0 | 0 |
| ASTRO2006475 | 0 | 0 | 0 | 0 | 0 | 0 | 16.519 |
| ASTRO2007948 | 5.009 | 0 | 0 | 2.555 | 0 | 0 | 0 |
| ASTRO2008040 | 23.959 | 0 | 0 | 12.222 | 0 | 0 | 0 |
| ASTRO2008216 | 0 | 0 | 0 | 3.256 | 0 | 0 | 0 |
| ASTRO2008972 | 0 | 0 | 0 | 0 | 0 | 0 | 17.271 |
| ASTRO2009068 | 5.658 | 0 | 0 | 0 | 21.061 | 0 | 0 |
| ASTRO2010072 | 0 | 39.838 | 0 | 0 | 0 | 0 | 0 |
| ASTRO2010799 | 0 | 0 | 50.556 | 0 | 0 | 0 | 0 |
| ASTRO2010819 | 4.037 | 0 | 2.384 | 4.119 | 0 | 0 | 0 |
| ASTRO2011422 | 0 | 0 | 0 | 7.401 | 0 | 0 | 0 |
| ASTRO2012552 | 0 | 0 | 0 | 0 | 0 | 0 | 1.639 |
| ASTRO2013585 | 1.462 | 0 | 0 | 0.995 | 0 | 0 | 0 |
| ASTRO2014135 | 0 | 0 | 12.305 | 0 | 0 | 0 | 0 |
| ASTRO2014174 | 0 | 0 | 0 | 22.748 | 0 | 0 | 0 |
| ASTRO2014392 | 0 | 33.906 | 0 | 0 | 0 | 0 | 0 |
| ASTRO2014576 | 0 | 0 | 0 | 0 | 42.066 | 0 | 0 |
| ASTRO2014863 | 0 | 0 | 11.968 | 0 | 12.574 | 0 | 0 |
| ASTRO2015328 | 7.611 | 0 | 0 | 3.883 | 0 | 0 | 4.220 |
| ASTRO2016114 | 14.497 | 0 | 0 | 0 | 0 | 0 | 0 |
| ASTRO2016491 | 2.014 | 1.155 | 10.704 | 4.110 | 5.623 | 3.632 | 1.117 |
| ASTRO2018169 | 0 | 2.167 | 0 | 1.927 | 0 | 0 | 0 |
| ASTRO2018540 | 0 | 0 | 0 | 1.587 | 0 | 0 | 0 |
| ASTRO3000482 | 0 | 0 | 0 | 0 | 0 | 0 | 24.247 |
| BGGI11000123 | 0.701 | 0 | 0 | 0.715 | 0 | 0 | 0.777 |
| BGGI12000054 | 0.236 | 3.782 | 3.337 | 0.240 | 0.877 | 0 | 1.045 |
| BGGI12000616 | 0 | 2.051 | 0 | 0 | 0 | 3.224 | 0.991 |
| BGGI12000839 | 0.737 | 0 | 0 | 0.752 | 0 | 0 | 0 |
| BGGI12001002 | 0 | 0 | 0 | 1.811 | 6.607 | 0 | 0 |
| BGGI12001075 | 0 | 0 | 0 | 2.020 | 0 | 0 | 0 |
| BGGI12001682 | 2.065 | 16.585 | 7.317 | 0 | 3.844 | 7.447 | 2.290 |
| BGGI12001714 | 2.550 | 0 | 0 | 0 | 0 | 0 | 0 |
| BLADE2001371 | 0 | 0 | 0 | 12.691 | 0 | 0 | 0 |
| BLADE2002738 | 0 | 0 | 0 | 0 | 0 | 0 | 13.644 |
| BLADE2004849 | 0 | 0 | 0 | 0 | 0 | 0 | 8.220 |
| BLADE2006412 | 12.143 | 0 | 0 | 6.194 | 0 | 0 | 0 |
| BLADE2006830 | 1.681 | 0 | 0 | 0 | 0 | 0 | 0 |
| BLADE2007043 | 12.470 | 0 | 0 | 0 | 0 | 0 | 0 |
| BLADE2007589 | 0 | 0 | 0 | 0 | 0 | 0 | 5.482 |
| BLADE2007935 | 0 | 0 | 0 | 12.691 | 0 | 0 | 0 |
| BLADE2008398 | 12.401 | 0 | 0 | 18.978 | 0 | 0 | 13.752 |
| BNGH41000098 | 7.604 | 0 | 8.980 | 5.172 | 0 | 0 | 16.866 |
| BNGH41000137 | 13.016 | 0 | 0 | 0 | 0 | 0 | 9.623 |
| BNGH41000190 | 0 | 0 | 0 | 0 | 0 | 0 | 2.806 |
| BNGH41000198 | 0 | 0 | 0 | 2.120 | 0 | 14.982 | 2.304 |
| BNGH42000360 | 0 | 0 | 0 | 0 | 0 | 1.134 | 0 |
| BNGH42000532 | 9.457 | 0 | 0 | 0 | 0 | 0 | 0 |
| BNGH42000739 | 12.444 | 2.855 | 6.612 | 26.026 | 0 | 0 | 5.520 |
| BNGH42001247 | 0 | 0 | 0 | 0 | 0 | 18.969 | 0 |
| BNGH42002168 | 0 | 0 | 0 | 1.357 | 0 | 0 | 1.475 |
| BNGH42002387 | 0 | 0 | 0 | 0 | 0 | 0 | 11.705 |
| BNGH42005968 | 0 | 0 | 0 | 0 | 0 | 0 | 2.547 |
| BNGH42006135 | 6.602 | 0 | 0 | 8.083 | 0 | 0 | 4.393 |
| BNGH42007460 | 4.031 | 0 | 3.570 | 1.028 | 0 | 0 | 0 |
| BNGH42007709 | 0.230 | 2.112 | 3.262 | 0.470 | 0.857 | 4.150 | 0 |
| BNGH42007788 | 0 | 0 | 0 | 3.141 | 0 | 0 | 0 |
| BNGH42008603 | 13.017 | 6.637 | 0 | 5.903 | 5.383 | 0 | 1.604 |
| BNGH42008743 | 0 | 0 | 0 | 3.141 | 0 | 0 | 0 |
| BNGH42009025 | 0 | 0 | 0 | 0 | 10.378 | 0 | 0 |
| BRACE1000020 | 0 | 0 | 0 | 11.484 | 0 | 0 | 0 |
| BRACE1000042 | 0 | 0 | 5.393 | 6.212 | 5.666 | 0 | 1.688 |
| BRACE1000051 | 17.925 | 0 | 0 | 0 | 0 | 0 | 0 |
| BRACE1000065 | 0 | 0 | 0 | 0 | 0 | 0 | 43.859 |
| BRACE1000070 | 7.151 | 4.101 | 0 | 0 | 0 | 0 | 1.982 |
| BRACE1000073 | 0 | 0 | 44.809 | 0 | 0 | 0 | 0 |
| BRACE1000101 | 0 | 0 | 0 | 0 | 0 | 40.349 | 0 |
| BRACE1000115 | 0.562 | 1.718 | 0.663 | 0 | 2.787 | 1.350 | 0 |
| BRACE1000131 | 0 | 0 | 3.690 | 1.063 | 3.877 | 0 | 2.310 |
| BRACE1000159 | 0 | 8.472 | 0 | 0 | 0 | 0 | 2.048 |
| BRACE1000169 | 0 | 0 | 0 | 20.683 | 0 | 0 | 0 |
| BRACE1000186 | 4.324 | 0 | 0 | 2.206 | 0 | 7.796 | 0 |
| BRACE1000187 | 10.796 | 0 | 0 | 8.261 | 0 | 0 | 17.958 |
| BRACE1000258 | 31.956 | 0 | 0 | 0 | 0 | 0 | 0 |
| BRACE2000109 | 0 | 0 | 0 | 0 | 0 | 0 | 60.975 |
| BRACE2000141 | 34.735 | 0 | 0 | 35.438 | 0 | 0 | 0 |
| BRACE2000245 | 0 | 0 | 0 | 0 | 0 | 0 | 39.230 |
| BRACE2000280 | 0 | 0 | 0 | 19.121 | 0 | 67.577 | 0 |
| BRACE2000300 | 15.947 | 0 | 18.831 | 10.846 | 0 | 0 | 5.895 |
| BRACE2000307 | 1.178 | 2.702 | 0 | 2.403 | 0 | 0 | 1.306 |
| BRACE2000331 | 58.488 | 0 | 0 | 0 | 0 | 0 | 0 |
| BRACE2000332 | 0 | 0 | 0 | 0 | 0 | 0 | 6.020 |
| BRACE2000347 | 0 | 5.931 | 0 | 0 | 9.622 | 0 | 0 |
| BRACE2000525 | 0 | 7.455 | 5.756 | 3.315 | 0 | 0 | 3.604 |
| BRACE2000565 | 0 | 16.454 | 0 | 0 | 0 | 0 | 7.953 |
| BRACE2000698 | 0 | 0 | 30.481 | 0 | 0 | 0 | 0 |
| BRACE2000733 | 0 | 0 | 0 | 0 | 65.597 | 0 | 0 |
| BRAGE2000750 | 0 | 0 | 0 | 0 | 0 | 0 | 22.823 |
| BRACE2000864 | 1.055 | 2.422 | 3.739 | 0.538 | 1.964 | 9.515 | 0 |
| BRACE2000893 | 4.519 | 0 | 0 | 0 | 0 | 0 | 5.012 |
| BRACE2000975 | 13.452 | 1.470 | 2.269 | 11.110 | 2.384 | 4.619 | |
| 12.787 | | | | | | | |
| BRACE2001065 | 0 | 0 | 0 | 0 | 0 | 0 | 7.364 |
| BRACE2001070 | 0 | 0 | 0 | 0 | 83.985 | 0 | 0 |
| BRACE2001143 | 0 | 0 | 0 | 0 | 0 | 5.356 | 0 |
| BRACE2001154 | 0 | 0 | 0 | 10.840 | 0 | 0 | 0 |
| BRACE2001188 | 0 | 0 | 0 | 0 | 0 | 0 | 3.130 |
| BRACE2001197 | 0 | 0 | 0 | 58.973 | 0 | 0 | 0 |
| BRACE2001236 | 0 | 0 | 0 | 0 | 0 | 64.877 | 0 |
| BRACE2001477 | 6.878 | 0 | 0 | 10.526 | 0 | 0 | 11.442 |
| BRACE2001496 | 0 | 0 | 17.187 | 0 | 18.057 | 17.493 | 0 |
| BRACE2001497 | 3.727 | 0 | 0 | 7.604 | 0 | 0 | 6.199 |
| BRACE2001534 | 0 | 7.077 | 0 | 3.147 | 0 | 0 | 0 |
| BRACE2001673 | 0 | 1.076 | 0 | 0 | 0 | 2.536 | 0.260 |
| BRACE2001692 | 1.817 | 0 | 0 | 0 | 0 | 0 | 0 |
| BRACE2001709 | 0 | 7.445 | 0 | 0 | 12.079 | 23.403 | 3.599 |
| BRACE2001711 | 3.890 | 0 | 0 | 0 | 0 | 0 | 0 |
| BRACE2001726 | 0 | 0 | 0 | 0 | 0 | 0 | 39.237 |
| BRACE2001834 | 7.591 | 0 | 26.891 | 19.361 | 0 | 13.685 | 0 |
| BRACE2001892 | 7.573 | 4.865 | 2.146 | 12.053 | 4.510 | 2.184 | 9.742 |
| BRACE2001944 | 6.273 | 0 | 0 | 0 | 23.348 | 0 | 0 |
| BRACE2001971 | 2.243 | 10.292 | 15.892 | 0 | 0 | 0 | 2.487 |
| BRACE2002050 | 5.800 | 6.653 | 10.273 | 5.917 | 0 | 0 | 6.432 |
| BRACE2002091 | 2.112 | 0 | 0 | 2.154 | 0 | 0 | 0 |
| BRACE2002356 | 0 | 0 | 0 | 18.538 | 0 | 0 | 0 |
| BRACE2002392 | 0 | 15.817 | 12.212 | 5.275 | 0 | 6.215 | 1.911 |
| BRACE2002478 | 0 | 0 | 0 | 14.787 | 0 | 0 | 0 |
| BRACE2002495 | 0 | 0 | 0 | 0 | 0 | 83.553 | 0 |
| BRACE2002582 | 6.533 | 0 | 0 | 13.331 | 0 | 47.115 | 0 |
| BRACE2002585 | 0 | 57.190 | 0 | 0 | 0 | 0 | 0 |
| BRACE2002606 | 0 | 0 | 0 | 5.468 | 0 | 0 | 0 |
| BRACE2002677 | 0 | 0 | 0 | 0 | 0 | 6.961 | 0 |
| BRACE2002682 | 19.147 | 0 | 0 | 19.534 | 0 | 0 | 0 |
| BRACE2002803 | 0 | 0 | 0 | 0 | 0 | 0 | 20.206 |
| BRACE2002812 | 5.877 | 0 | 0 | 0 | 21.873 | 0 | 19.551 |
| BRACE2002860 | 0 | 0 | 0 | 0 | 0 | 0 | 2.792 |
| BRACE2002896 | 14.988 | 0 | 53.097 | 0 | 0 | 0 | 0 |
| BRACE2003037 | 4.318 | 9.907 | 15.298 | 6.609 | 0 | 0 | 2.394 |
| BRACE2003078 | 0 | 0 | 0 | 0 | 0 | 58.364 | 0 |
| BRACE2003097 | 0 | 0 | 0 | 58.973 | 0 | 0 | 0 |
| BRACE2003172 | 0 | 0.746 | 2.303 | 0.663 | 0 | 1.172 | 1.442 |
| BRACE2003217 | 0 | 0 | 15.270 | 0 | 0 | 0 | 0 |
| BRACE2003254 | 0 | 0 | 0 | 0 | 0 | 3.264 | 4.016 |
| BRACE2003527 | 0 | 5.914 | 0 | 0 | 9.594 | 0 | 0 |
| BRACE2003539 | 0 | 0 | 0 | 0 | 0 | 0 | 60.975 |
| BRACE2003598 | 0 | 0 | 0 | 0 | 0 | 0 | 60.975 |
| BRACE2003609 | 9.090 | 0 | 0 | 0 | 0 | 0 | 0 |
| BRACE2003700 | 0 | 0 | 0 | 14.012 | 0 | 0 | 15.230 |
| BRACE2003785 | 0.972 | 6.688 | 0 | 6.940 | 0 | 0 | 0 |
| BRACE2003788 | 0 | 0 | 0 | 39.793 | 0 | 0 | 0 |
| BRACE2003800 | 0 | 0 | 0 | 0 | 0 | 0 | 30.394 |
| BRACE2003827 | 0 | 0 | 0 | 0.894 | 3.261 | 0 | 0 |
| BRACE2003845 | 0 | 0 | 0 | 4.648 | 0 | 0 | 0 |
| BRACE2003846 | 3.259 | 0 | 0 | 0.665 | 2.426 | 0 | 0 |
| BRACE2003892 | 2.182 | 0 | 0 | 2.227 | 0 | 0 | 2.420 |
| BRACE2003904 | 0 | 0 | 0 | 0 | 0 | 9.348 | 0 |
| BRACE2003905 | 15.814 | 0 | 0 | 0 | 0 | 0 | 0 |
| BRACE2003944 | 0.518 | 1.188 | 1.834 | 0 | 1.927 | 1.867 | 0 |
| BRACE2003982 | 0 | 9.441 | 14.578 | 4.198 | 0 | 0 | 0 |
| BRACE2005083 | 0 | 0 | 0 | 24.542 | 0 | 0 | 0 |
| BRACE2005087 | 2.075 | 0 | 0 | 6.350 | 0 | 0 | 0 |
| BRACE2005099 | 2.659 | 12.199 | 9.418 | 2.712 | 9.895 | 19.172 | 4.422 |
| BRACE2005151 | 2.101 | 0 | 0 | 0 | 0 | 0 | 4.660 |
| BRACE2005193 | 0 | 0 | 0 | 58.973 | 0 | 0 | 0 |
| BRACE2005231 | 6.385 | 0 | 0 | 9.771 | 0 | 0 | 3.540 |
| BRACE2005251 | 21.139 | 0 | 0 | 0 | 0 | 0 | 0 |
| BRACE2005253 | 0 | 0 | 0 | 0 | 0 | 0 | 60.975 |
| BRACE2005290 | 0 | 0 | 0 | 25.958 | 0 | 0 | 0 |
| BRACE2005395 | 0 | 0 | 0 | 0 | 0 | 0 | 10.456 |
| BRACE2005408 | 0 | 21.821 | 0 | 9.704 | 0 | 0 | 0 |
| BRACE2005434 | 0 | 0 | 0 | 0 | 0 | 4.972 | 0 |
| BRACE2005448 | 6.800 | 0 | 0 | 0 | 0 | 0 | 0 |
| BRACE2005457 | 58.488 | 0 | 0 | 0 | 0 | 0 | 0 |
| BRACE2005642 | 7.307 | 0 | 0 | 0 | 0 | 0 | 8.103 |
| BRACE2005681 | 16.971 | 4.581 | 0 | 20.370 | 7.431 | 7.199 | 3.321 |
| BRACE2005719 | 0 | 6.814 | 0 | 3.030 | 11.054 | 0 | 0 |
| BRACE2005858 | 41.331 | 0 | 0 | 21.084 | 0 | 0 | 22.917 |
| BRACE2006055 | 4.001 | 0 | 0 | 0 | 0 | 0 | 2.218 |
| BRACE2006075 | 0 | 0 | 0 | 0 | 0 | 58.507 | 0 |
| BRACE2006084 | 0 | 14.084 | 0 | 0 | 0 | 11.067 | 0 |
| BRACE2006089 | 16.309 | 0 | 0 | 0 | 0 | 0 | 0 |
| BRACE2006167 | 8.432 | 9.673 | 22.404 | 4.301 | 0 | 7.601 | 7.013 |
| BRACE2006258 | 0.819 | 3.758 | 5.804 | 0 | 6.097 | 0 | 0 |
| BRACE2006264 | 0 | 0 | 29.900 | 0 | 0 | 0 | 9.360 |
| BRACE2006281 | 0 | 0 | 0 | 0 | 0 | 10.896 | 0 |
| BRACE2006344 | 0 | 8.201 | 0 | 0 | 0 | 0 | 0 |
| BRACE2006354 | 0 | 14.276 | 0 | 12.697 | 0 | 0 | 0 |
| BRACE2006363 | 0 | 21.263 | 0 | 0 | 0 | 0 | 0 |
| BRACE2006380 | 0 | 0 | 0 | 41.817 | 0 | 0 | 0 |
| BRACE2006397 | 0.295 | 0.677 | 0 | 0 | 0 | 1.064 | 0.327 |
| BRACE2006514 | 13.031 | 0 | 0 | 5.318 | 3.233 | 3.132 | 5.780 |
| BRACE2006537 | 0 | 7.407 | 3.813 | 1.098 | 4.006 | 3.881 | 2.387 |
| BRACE2006636 | 13.710 | 0 | 0 | 27.974 | 0 | 0 | 15.203 |
| BRACE2006743 | 8.892 | 0 | 0 | 4.536 | 33.098 | 0 | 0 |
| BRACE2006753 | 0 | 14.107 | 0 | 12.547 | 0 | 0 | 0 |
| BRACE2006821 | 0 | 72.016 | 0 | 0 | 0 | 6.658 | 0 |
| BRACE2006859 | 0 | 0 | 0 | 0 | 0 | 0 | 11.267 |
| BRACE2006871 | 0 | 0 | 3.542 | 0 | 0 | 0 | 0 |
| BRACE2006937 | 4.015 | 3.070 | 0 | 1.365 | 4.981 | 4.826 | 1.484 |
| BRACE2006982 | 0 | 11.904 | 9.191 | 0 | 4.828 | 14.032 | 0 |
| BRACE2007013 | 0 | 47.046 | 0 | 10.461 | 0 | 0 | 0 |
| BRACE2007064 | 6.500 | 3.728 | 0 | 4.973 | 0 | 5.859 | 5.406 |
| BRACE2007089 | 0 | 1.943 | 0 | 0 | 0 | 0 | 0 |
| BRACE2007109 | 1.909 | 19.709 | 0 | 0.974 | 3.553 | 3.442 | 0 |
| BRACE2007191 | 0.687 | 1.576 | 0 | 0.701 | 0 | 0 | 3.809 |
| BRACE2007201 | 0 | 0 | 0 | 58.973 | 0 | 0 | 0 |
| BRACE2007254 | 0 | 17.078 | 0 | 0 | 0 | 0 | 0 |
| BRACE2007401 | 9.138 | 4.193 | 6.474 | 1.865 | 0 | 0 | 0 |
| BRACE2007518 | 9.078 | 0 | 0 | 2.315 | 0 | 0 | 10.067 |
| BRACE2007522 | 0 | 0 | 0 | 58.973 | 0 | 0 | 0 |
| BRACE2007567 | 0 | 0 | 0 | 2.831 | 0 | 5.003 | 0 |
| BRACE2007577 | 0.979 | 0 | 0 | 0.999 | 0 | 0 | 1.086 |
| BRACE2007641 | 3.135 | 3.596 | 0 | 4.798 | 5.835 | 0 | 3.477 |
| BRACE2007663 | 0 | 0 | 0 | 32.393 | 0 | 0 | 0 |
| BRACE2007671 | 0 | 0 | 16.431 | 0 | 11.509 | 5.575 | 0 |
| BRACE2007708 | 6.375 | 9.751 | 15.056 | 0 | 0 | 0 | 2.357 |
| BRACE2007767 | 0 | 0 | 0 | 0 | 0 | 0 | 3.452 |
| BRACE2007768 | 0 | 0 | 0 | 9.762 | 0 | 0 | 0 |
| BRACE2007798 | 2.069 | 9.492 | 7.329 | 2.111 | 19.249 | 18.648 | 0 |
| BRACE2007832 | 0 | 0 | 0 | 25.608 | 0 | 0 | 0 |
| BRACE2007882 | 13.172 | 0 | 0 | 6.719 | 0 | 0 | 0 |
| BRACE2007937 | 12.699 | 0 | 0 | 2.591 | 0 | 9.158 | 2.817 |
| BRACE2007952 | 14.547 | 0 | 0 | 19.789 | 18.048 | 0 | 5.378 |
| BRACE2008097 | 0 | 0 | 17.008 | 0 | 0 | 0 | 0 |
| BRACE2008216 | 0 | 30.375 | 0 | 13.508 | 0 | 0 | 0 |
| BRACE2008251 | 0 | 31.081 | 0 | 0 | 0 | 0 | 0 |
| BRACE2008358 | 0 | 0 | 0 | 2.312 | 0 | 0 | 0 |
| BRACE2008380 | 0 | 8.965 | 13.843 | 0 | 14.544 | 21.134 | 0 |
| BRACE2008385 | 0 | 0 | 7.399 | 0 | 0 | 7.531 | 0 |
| BRACE2008399 | 0 | 0 | 0 | 5.647 | 0 | 0 | 0 |
| BRACE2008402 | 0 | 0 | 0 | 4.278 | 0 | 0 | 0 |
| BRACE2008553 | 0 | 0 | 0 | 0 | 0 | 14.996 | 0 |
| BRACE2008653 | 9.493 | 2.420 | 0 | 5.381 | 0 | 0 | 5.849 |
| BRACE2008706 | 0 | 0 | 0 | 0 | 0 | 0 | 3.644 |
| BRACE2008708 | 0 | 0 | 0 | 0 | 0 | 50.398 | 0 |
| BRACE2008857 | 58.488 | 0 | 0 | 0 | 0 | 0 | 0 |
| BRACE2008941 | 19.988 | 0 | 0 | 0 | 0 | 0 | 0 |
| BRACE2008968 | 0 | 0 | 0 | 1.186 | 0 | 2.097 | 0.645 |
| BRACE2009037 | 0.584 | 2.680 | 0 | 0.894 | 0 | 0 | 1.295 |
| BRACE2009045 | 0 | 0 | 0 | 12.286 | 0 | 0 | 13.354 |
| BRACE2009291 | 1.323 | 3.035 | 0 | 0 | 0 | 0 | 0 |
| BRACE2009307 | 0 | 0 | 0 | 15.110 | 0 | 0 | 16.424 |
| BRACE2009366 | 0 | 0 | 12.799 | 3.686 | 0 | 0 | 0 |
| BRACE2009483 | 8.786 | 0 | 0 | 0 | 0 | 0 | 9.744 |
| BRACE2009517 | 0 | 0 | 0 | 2.767 | 0 | 0 | 0 |
| BRACE2009566 | 0 | 0 | 67.189 | 19.350 | 0 | 0 | 0 |
| BRACE2010489 | 63.510 | 0 | 0 | 10.799 | 0 | 0 | 0 |
| BRACE2010651 | 18.479 | 0 | 0 | 0 | 0 | 0 | 0 |
| BRACE2010669 | 19.124 | 0 | 0 | 6.504 | 0 | 0 | 7.069 |
| BRACE2010753 | 0 | 0 | 66.077 | 0 | 0 | 0 | 20.684 |
| BRACE2011183 | 0 | 0 | 0 | 13.116 | 0 | 0 | 14.257 |
| BRACE2012317 | 23.331 | 0 | 0 | 34.304 | 0 | 0 | 3.805 |
| BRACE2012528 | 3.794 | 7.616 | 0 | 4.354 | 0 | 6.840 | 13.673 |
| BRACE2012814 | 3.067 | 0 | 0 | 0 | 0 | 0 | 1.701 |
| BRACE2013855 | 0 | 0 | 28.415 | 0 | 0 | 0 | 0 |
| BRACE2013911 | 7.517 | 0 | 0 | 0 | 0 | 0 | 16.672 |
| BRACE2014257 | 12.401 | 0 | 0 | 0 | 0 | 0 | 0 |
| BRACE2014306 | 0 | 0 | 34.486 | 0 | 0 | 17.550 | 5.398 |
| BRACE2014657 | 15.451 | 0 | 0 | 0 | 0 | 0 | 0 |
| BRACE2014746 | 0 | 32.019 | 0 | 0 | 0 | 0 | 0 |
| BRACE2014850 | 7.500 | 0 | 0 | 0 | 0 | 0 | 8.317 |
| BRACE2015058 | 0 | 0 | 0 | 8.947 | 0 | 0 | 19.449 |
| BRACE2015132 | 0 | 0 | 58.366 | 0 | 0 | 0 | 0 |
| BRACE2015287 | 0 | 0 | 8.135 | 0 | 0 | 0 | 2.547 |
| BRACE2015368 | 58.488 | 0 | 0 | 0 | 0 | 0 | 0 |
| BRACE2015412 | 12.174 | 0 | 0 | 0 | 0 | 0 | 0 |
| BRACE2016194 | 0 | 76.373 | 0 | 0 | 0 | 0 | 0 |
| BRACE2017359 | 0 | 0 | 0 | 41.817 | 0 | 0 | 0 |
| BRACE2017397 | 0 | 0 | 0 | 4.813 | 0 | 0 | 0 |
| BRACE2017574 | 0 | 0 | 0 | 0 | 0 | 0 | 5.682 |
| BRACE2017844 | 0 | 11.790 | 0 | 5.243 | 38.254 | 0 | 0 |
| BRACE2017934 | 0 | 0 | 0 | 0 | 0 | 33.326 | 0 |
| BRACE2018337 | 9.527 | 0 | 0 | 2.430 | 8.865 | 25.764 | 0 |
| BRACE2018736 | 0 | 0 | 0 | 0 | 0 | 0 | 44.853 |
| BRACE2018762 | 0 | 0 | 0 | 58.973 | 0 | 0 | 0 |
| BRACE2018847 | 39.016 | 0 | 0 | 0 | 0 | 0 | 21.634 |
| BRACE2019004 | 0 | 0 | 0 | 0 | 0 | 0 | 60.975 |
| BRACE2019147 | 0 | 0 | 0 | 2.185 | 7.971 | 0 | 0 |
| BRACE2019327 | 0 | 20.559 | 0 | 0 | 0 | 0 | 9.938 |
| BRACE2019348 | 0 | 0 | 0 | 0 | 0 | 0 | 25.772 |
| BRACE2019519 | 0 | 0 | 65.939 | 0 | 0 | 0 | 0 |
| BRACE2019877 | 0 | 13.934 | 0 | 0 | 0 | 21.900 | 6.735 |
| BRACE2021014 | 17.772 | 0 | 0 | 18.132 | 0 | 0 | 0 |
| BRACE2021708 | 29.608 | 0 | 0 | 0 | 0 | 0 | 0 |
| BRACE2021932 | 0 | 0 | 0 | 58.973 | 0 | 0 | 0 |
| BRACE2022333 | 17.756 | 0 | 0 | 18.115 | 0 | 0 | 19.690 |
| BRACE2022638 | 1.491 | 1.710 | 0 | 0.760 | 0 | 2.687 | 0.827 |
| BRACE2023223 | 0 | 0 | 0 | 37.268 | 0 | 0 | 0 |
| BRACE2023718 | 32.097 | 0 | 0 | 16.373 | 0 | 0 | 0 |
| BRACE2024781 | 58.488 | 0 | 0 | 0 | 0 | 0 | 0 |
| BRACE2025437 | 0 | 0 | 0 | 0 | 0 | 11.008 | 0 |
| BRACE2026142 | 0 | 0 | 0 | 0 | 83.985 | 0 | 0 |
| BRACE2027018 | 0 | 0 | 83.309 | 0 | 0 | 0 | 0 |
| BRACE2027643 | 0 | 33.045 | 0 | 0 | 0 | 0 | 0 |
| BRACE2027767 | 58.488 | 0 | 0 | 0 | 0 | 0 | 0 |
| BRACE2027896 | 25.575 | 0 | 0 | 0 | 0 | 0 | 0 |
| BRACE2028392 | 0 | 0 | 18.058 | 0 | 37.945 | 36.760 | 0 |
| BRACE2028636 | 0 | 0 | 0 | 13.653 | 0 | 0 | 0 |
| BRACE2030341 | 0 | 0 | 0 | 7.057 | 0 | 0 | 0 |
| BRACE2031154 | 0 | 0 | 0 | 0 | 0 | 0 | 60.975 |
| BRACE2032477 | 0 | 0 | 0 | 5.685 | 0 | 0 | 0 |
| BRACE2032502 | 0 | 0 | 0 | 0 | 0 | 0 | 60.975 |
| BRACE2035381 | 10.177 | 0 | 0 | 20.766 | 0 | 0 | 11.286 |
| BRACE2037132 | 58.488 | 0 | 0 | 0 | 0 | 0 | 0 |
| BRACE2037295 | 3.280 | 0 | 0 | 0 | 12.209 | 0 | 0 |
| BRACE2039624 | 0.967 | 0 | 0 | 0.987 | 3.601 | 3.488 | 1.073 |
| BRACE2044105 | 0 | 0 | 0 | 0 | 0 | 72.562 | 0 |
| BRACE2044286 | 18.667 | 0 | 0 | 0 | 0 | 0 | 0 |
| BRACE2045954 | 27.309 | 0 | 0 | 0 | 0 | 0 | 0 |
| BRACE3003192 | 58.488 | 0 | 0 | 0 | 0 | 0 | 0 |
| BRACE3004046 | 0 | 0 | 0 | 58.973 | 0 | 0 | 0 |
| BRACE3004371 | 11.270 | 0 | 0 | 7.666 | 0 | 0 | 4.166 |
| BRACE3004767 | 58.488 | 0 | 0 | 0 | 0 | 0 | 0 |
| BRACE3005499 | 31.276 | 0 | 0 | 0 | 0 | 0 | 0 |
| BRACE3005760 | 45.047 | 0 | 0 | 22.980 | 0 | 0 | 0 |
| BRACE3005938 | 1.101 | 0 | 3.901 | 4.494 | 4.099 | 0 | 4.885 |
| BRACE3005989 | 0 | 0.536 | 0 | 0.239 | 0.870 | 0.843 | 0 |
| BRACE3007258 | 13.014 | 0 | 0 | 13.277 | 0 | 0 | 28.863 |
| BRACE3007480 | 19.471 | 0 | 0 | 52.973 | 0 | 0 | 7.197 |
| BRACE3008384 | 0 | 0 | 0 | 0 | 0 | 0 | 43.859 |
| BRACE3009237 | 18.139 | 0 | 0 | 37.013 | 0 | 0 | 20.116 |
| BRACE3009416 | 0 | 0 | 0 | 29.118 | 0 | 0 | 0 |
| BRACE3009724 | 58.488 | 0 | 0 | 0 | 0 | 0 | 0 |
| BRACE3010397 | 0 | 0 | 0 | 0 | 0 | 0 | 75.757 |
| BRACE3010428 | 6.868 | 0 | 0 | 2.336 | 4.261 | 0 | 2.539 |
| BRACE3010438 | 5.372 | 0 | 0 | 0 | 0 | 0 | 3.971 |
| BRACE3011271 | 11.036 | 0 | 0 | 11.259 | 0 | 0 | 0 |
| BRACE3011421 | 28.251 | 0 | 0 | 0 | 0 | 0 | 0 |
| BRACE3013780 | 0 | 0 | 0 | 0 | 17.852 | 0 | 0 |
| BRACE3016122 | 58.488 | 0 | 0 | 0 | 0 | 0 | 0 |
| BRACE3018963 | 0 | 0 | 0 | 58.973 | 0 | 0 | 0 |
| BRACE3019071 | 28.341 | 0 | 0 | 0 | 0 | 0 | 31.429 |
| BRACE3019941 | 0 | 0 | 0 | 0 | 0 | 0 | 6.480 |
| BRACE3022312 | 0 | 0 | 0 | 0 | 0 | 0 | 37.734 |
| BRACE3022340 | 58.488 | 0 | 0 | 0 | 0 | 0 | 0 |
| BRACE3025719 | 0 | 0 | 0 | 0 | 67.040 | 0 | 0 |
| BRACE3026735 | 24.173 | 0 | 0 | 0 | 0 | 0 | 0 |
| BRACE3026802 | 0 | 0 | 0 | 0 | 63.610 | 0 | 0 |
| BRACE3027478 | 0 | 0 | 0 | 0 | 0 | 0 | 35.497 |
| BRACE3027931 | 0 | 0 | 0 | 34.325 | 0 | 0 | 0 |
| BRACE3029021 | 0 | 0 | 0 | 74.193 | 0 | 0 | 0 |
| BRACE3030866 | 58.488 | 0 | 0 | 0 | 0 | 0 | 0 |
| BRACE3031185 | 58.488 | 0 | 0 | 0 | 0 | 0 | 0 |
| BRACE3031838 | 58.488 | 0 | 0 | 0 | 0 | 0 | 0 |
| BRACE3032427 | 58.488 | 0 | 0 | 0 | 0 | 0 | 0 |
| BRACE3032631 | 12.612 | 0 | 0 | 0 | 0 | 45.475 | 0 |
| BRACE3036156 | 0 | 0 | 0 | 44.660 | 0 | 0 | 24.272 |
| BRACE3039358 | 41.331 | 0 | 0 | 21.084 | 0 | 0 | 22.917 |
| BRACE3040863 | 17.888 | 0 | 0 | 18.250 | 0 | 0 | 0 |
| BRACE3042326 | 0 | 0 | 0 | 35.796 | 0 | 0 | 0 |
| BRACE3042432 | 58.488 | 0 | 0 | 0 | 0 | 0 | 0 |
| BRACE3045078 | 0 | 0 | 0 | 41.817 | 0 | 0 | 0 |
| BRACE3045981 | 26.434 | 0 | 0 | 0 | 0 | 0 | 0 |
| BRACE3046450 | 21.984 | 0 | 0 | 0 | 0 | 0 | 0 |
| BRALZ2000988 | 15.571 | 0 | 0 | 10.591 | 0 | 0 | 5.756 |
| BRALZ2001834 | 0 | 0 | 0 | 0 | 0 | 0 | 23.483 |
| BRALZ2006288 | 0 | 0 | 0 | 11.126 | 0 | 0 | 0 |
| BRALZ2008617 | 8.102 | 0 | /0 | 0 | 0 | 0 | 0 |
| BRALZ2008696 | 21.676 | 0 | 0 | 0 | 0 | 0 | 0 |
| BRALZ2009446 | 0 | 10.697 | 0 | 0 | 17.354 | 16.812 | 0 |
| BRALZ2009482 | 0 | 0 | 1.316 | 0 | 0 | 0 | 3.297 |
| BRALZ2009486 | 0 | 24.097 | 0 | /0 | 0 | 0 | 0 |
| BRALZ2011796 | 5.511 | 0 | 19.525 | 0 | 20.514 | 0 | 6.112 |
| BRALZ2011880 | 3.049 | 0 | 0 | 0 | 0 | 0 | 0 |
| BRALZ2018834 | 0 | 0 | 22.106 | 6.366 | 0 | 0 | 0 |
| BRALZ2019047 | 0 | 0 | 0 | 0 | 0 | 0 | 23.483 |
| BRAMY1000173 | 21.150 | 0 | 0 | 0 | 0 | 0 | 11.727 |
| BRAMY2000025 | 0 | 36.202 | 0 | 16.099 | 0 | 0 | 0 |
| BRAMY2000052 | 0 | 0 | 0 | 0 | 0 | 39.047 | 0 |
| BRAMY2000354 | 0 | 0 | 0 | 1.402 | 0 | 0 | 0 |
| BRAMY2000388 | 6.118 | 0 | 0 | 6.242 | 0 | 0 | 0 |
| BRAMY2000411 | 0 | 0 | 0 | 12.647 | 23.069 | 0 | 0 |
| BRAMY2000508 | 35.193 | 0 | 0 | 0 | 0 | 0 | 0 |
| BRAMY2000562 | 3.843 | 0 | 0 | 9.803 | 0 | 0 | 6.393 |
| BRAMY2000585 | 0 | 0 | 0 | 0 | 0 | 0 | 2.724 |
| BRAMY2000893 | 0 | 0 | 0 | 0 | 5.664 | 0 | 0 |
| BRAMY2002369 | 24.474 | 0 | 0 | 24.970 | 0 | 0 | 27.141 |
| BRAMY2002479 | 0 | 0 | 0 | 0 | 0 | 78.159 | 0 |
| BRAMY2002621 | 0 | 0 | 0 | 0 | 0 | 0 | 39.237 |
| BRAMY2002799 | 49.811 | 0 | 0 | 0 | 0 | 0 | 0 |
| BRAMY2003008 | 26.445 | 0 | 0 | 0 | 0 | 0 | 0 |
| BRAMY2004031 | 0 | 29.171 | 45.044 | 12.972 | 0 | 0 | 0 |
| BRAMY2004363 | 12.266 | 0 | 0 | 6.257 | 0 | 0 | 2.267 |
| BRAMY2004387 | 0 | 69.484 | 0 | 0 | 0 | 0 | 0 |
| BRAMY2004521 | 2.370 | 0 | 0 | 2.418 | 0 | 0 | 5.257 |
| BRAMY2004524 | 0 | 0 | 0 | 0 | 0 | 78.159 | 0 |
| BRAMY2005052 | 11.612 | 0 | 0 | 0 | 0 | 0 | 25.755 |
| BRAMY2005488 | 0 | 0 | 40.370 | 11.626 | 0 | 0 | 0 |
| BRAMY2006397 | 0 | 6.443 | 0 | 2.865 | 10.452 | 0 | 0 |
| BRAMY2007078 | 0 | 0 | 0 | 0 | 0 | 0 | 8.434 |
| BRAMY2007244 | 0 | 26.889 | 0 | 11.957 | 0 | 0 | 0 |
| BRAMY2007255 | 0 | 0 | 0 | 40.300 | 0 | 0 | 0 |
| BRAMY2007287 | 10.715 | 0 | 0 | 0 | 0 | 0 | 0 |
| BRAMY2007567 | 31.038 | 0 | 0 | 0 | 0 | 0 | 0 |
| BRAMY2007594 | 0 | 0 | 0 | 0 | 0 | 0 | 68.762 |
| BRAMY2007613 | 30.810 | 0 | 0 | 9.567 | 0 | 4.830 | 10.399 |
| BRAMY2008563 | 0 | 0 | 0 | 50.312 | 0 | 0 | 0 |
| BRAMY2008977 | 5.056 | 0 | 0 | 5.158 | 0 | 0 | 5.607 |
| BRAMY2009013 | 6.091 | 0 | 0 | 0 | 0 | 0 | 0 |
| BRAMY2009023 | 26.514 | 0 | 0 | 0 | 0 | 0 | 0 |
| BRAMY2009489 | 0 | 0 | 0 | 18.337 | 0 | 0 | 19.931 |
| BRAMY2009508 | 7.498 | 0 | 0 | 7.650 | 0 | 0 | 16.630 |
| BRAMY2009557 | 0 | 0 | 0 | 50.312 | 0 | 0 | 0 |
| BRAMY2009612 | 0 | 0 | 0 | 27.617 | 0 | 0 | 0 |
| BRAMY2009934 | 0 | 0 | 0 | 6.031 | 0 | 0 | 0 |
| BRAMY2010272 | 49.811 | 0 | 0 | 0 | 0 | 0 | 0 |
| BRAMY2010357 | 14.931 | 0 | 0 | 15.234 | 0 | 0 | 0 |
| BRAMY2010442 | 0 | 0 | 0 | 0 | 0 | 0 | 52.395 |
| BRAMY2010464 | 11.017 | 0 | 0 | 0 | 0 | 0 | 0 |
| BRAMY2011105 | 0 | 6.950 | 0 | 0 | 11.275 | 0 | 0 |
| BRAMY2011446 | 0 | 0 | 0 | 0 | 0 | 0 | 39.237 |
| BRAMY2011841 | 0 | 0 | 0 | 11.258 | 0 | 0 | 0 |
| BRAMY2011849 | 0 | 0 | 0 | 50.312 | 0 | 0 | 0 |
| BRAMY2012163 | 0 | 0 | 28.009 | 0 | 58.856 | 0 | 0 |
| BRAMY2012340 | 0 | 0 | 0 | 50.312 | 0 | 0 | 0 |
| BRAMY2012536 | 0 | 1.324 | 6.134 | 0.589 | 0 | 0 | 0 |
| BRAMY2012881 | 0 | 0 | 0 | 0 | 0 | 0 | 52.395 |
| BRAMY2013169 | 0 | 0 | 0 | 0 | 0 | 41.000 | 12.610 |
| BRAMY2013736 | 0 | 0 | 0 | 0 | 0 | 0 | 52.395 |
| BRAMY2014205 | 0 | 0 | 0 | 0 | 53.328 | 0 | 0 |
| BRAMY2014462 | 0 | 0 | 0 | 50.312 | 0 | 0 | 0 |
| BRAMY2014754 | 5.603 | 12.854 | 9.924 | 27.152 | 10.427 | 0 | 12.427 |
| BRAMY2015251 | 3.062 | 21.075 | 21.695 | 3.124 | 0 | 11.041 | 0 |
| BRAMY2015420 | 63.595 | 0 | 0 | 0 | 0 | 0 | 0 |
| BRAMY2015550 | 0 | 0 | 0 | 0 | 0 | 0 | 37.587 |
| BRAMY2015833 | 4.610 | 0 | 0 | 4.703 | 0 | 16.621 | 5.112 |
| BRAMY2015911 | 0 | 0 | 0 | 0 | 0 | 0 | 33.045 |
| BRAMY2016009 | 10.459 | 11.998 | 0 | 16.006 | 0 | 0 | 5.799 |
| BRAMY2016251 | 0 | 16.499 | 0 | 0 | 0 | 0 | 0 |
| BRAMY2016325 | 11.304 | 0 | 0 | 3.844 | 0 | 0 | 4.178 |
| BRAMY2016892 | 14.520 | 0 | 0 | 44.442 | 0 | 0 | 16.102 |
| BRAMY2017864 | 0 | 0 | 0 | 14.097 | 51.427 | 0 | 0 |
| BRAMY2018295 | 0 | 0 | 63.596 | 18.315 | 0 | 0 | 0 |
| BRAMY2018635 | 27.163 | 0 | 0 | 27.712 | 0 | 0 | 0 |
| BRAMY2018785 | 0 | 0 | 24.091 | 6.938 | 0 | 0 | 0 |
| BRAMY2019111 | 0 | 69.484 | 0 | 0 | 0 | 0 | 0 |
| BRAMY2019300 | 49.811 | 0 | 0 | 0 | 0 | 0 | 0 |
| BRAMY2019468 | 34.185 | 0 | 0 | 23.251 | 0 | 0 | 12.637 |
| BRAMY2019554 | 1.596 | 0 | 0 | 4.885 | 2.970 | 0 | 0.885 |
| BRAMY2019600 | 16.148 | 0 | 0 | 0 | 0 | 0 | 8.954 |
| BRAMY2019710 | 0 | 0 | 0 | 0 | 0 | 0 | 52.395 |
| BRAMY2019963 | 20.428 | 0 | 0 | 6.947 | 0 | 0 | 7.551 |
| BRAMY2019989 | 11.571 | 0 | 0 | 10.821 | 0 | 0 | 4.277 |
| BRAMY2020106 | 0 | 0 | 0 | 0 | 0 | 40.308 | 0 |
| BRAMY2020427 | 0.592 | 1.358 | 0 | 1.812 | 0 | 4.270 | 1.313 |
| BRAMY2021276 | 0 | 0 | 0 | 0 | 0 | 0 | 52.395 |
| BRAMY2021825 | 0 | 0 | 0 | 33.611 | 0 | 0 | 0 |
| BRAMY2021867 | 0 | 0 | 0 | 0 | 0 | 0 | 39.237 |
| BRAMY2022160 | 0 | 0 | 0 | 51.413 | 0 | 0 | 0 |
| BRAMY2022168 | 0 | 0 | 0 | 13.801 | 0 | 0 | 30.003 |
| BRAMY2022493 | 2.653 | 0 | 0 | 2.707 | 0 | 0 | 0 |
| BRAMY2022525 | 22.053 | 0 | 0 | 0 | 0 | 0 | 0 |
| BRAMY2022723 | 0 | 0 | 0 | 0 | 0 | 0 | 39.237 |
| BRAMY2022786 | 3.743 | 2.863 | 4.421 | 1.273 | 4.644 | 8.999 | 0 |
| BRAMY2022980 | 30.533 | 0 | 54.084 | 0 | 0 | 0 | 0 |
| BRAMY2023115 | 0 | 0 | 0 | 50.312 | 0 | 0 | 0 |
| BRAMY2023406 | 0 | 0 | 23.388 | 0 | 24.572 | 0 | 0 |
| BRAMY2024004 | 0 | 6.346 | 9.799 | 0 | 0 | 0 | 3.067 |
| BRAMY2024312 | 0 | 0 | 0 | 50.312 | 0 | 0 | 0 |
| BRAMY2024390 | 0 | 0 | 0 | 35.652 | 0 | 0 | 0 |
| BRAMY2024416 | 4.525 | 0 | 0 | 9.233 | 0 | 0 | 35.125 |
| BRAMY2024593 | 12.891 | 0 | 0 | 13.152 | 47.980 | 0 | 0 |
| BRAMY2024711 | 0 | 0 | 43.436 | 0 | 0 | 44.210 | 0 |
| BRAMY2024827 | 0 | 0 | 0 | 4.071 | 0 | 0 | 0 |
| BRAMY2025121 | 0 | 37.987 | 0 | 16.893 | 0 | 0 | 0 |
| BRAMY2025272 | 0 | 0 | 0 | 0 | 0 | 0 | 52.395 |
| BRAMY2026168 | 32.087 | 0 | 0 | 0 | 0 | 0 | 35.583 |
| BRAMY2026533 | 0 | 42.945 | 0 | 38.195 | 0 | 0 | 0 |
| BRAMY2027114 | 0 | 0 | 0 | 0 | 0 | 0 | 19.060 |
| BRAMY2027587 | 0 | 0 | 0 | 13.623 | 0 | 0 | 0 |
| BRAMY2027717 | 20.585 | 0 | 0 | 12.601 | 0 | 0 | 50.221 |
| BRAMY2027739 | 0 | 0 | 0 | 0 | 0 | 4.852 | 0 |
| BRAMY2028183 | 0 | 0 | 0 | 0 | 0 | 67.737 | 0 |
| BRAMY2028269 | 0 | 37.447 | 0 | 0 | 0 | 0 | 0 |
| BRAMY2028516 | 0 | 0 | 0 | 0 | 14.680 | 0 | 0 |
| BRAMY2028740 | 4.655 | 2.136 | 9.895 | 4.749 | 0 | 0 | 5.162 |
| BRAMY2028796 | 0 | 0 | 0 | 1.517 | 0 | 0 | 0 |
| BRAMY2028914 | 0 | 0 | 0 | 0 | 0 | 0 | 19.645 |
| BRAMY2029988 | 0 | 0 | 0 | 12.017 | 0 | 0 | 13.062 |
| BRAMY2031317 | 0 | 0 | 0 | 16.004 | 0 | 0 | 34.791 |
| BRAMY2031516 | 14.772 | 0 | 0 | 7.536 | 0 | 0 | 0 |
| BRAMY2032087 | 2.422 | 0 | 0 | 1.236 | 0 | 0 | 2.686 |
| BRAMY2033914 | 49.811 | 0 | 0 | 0 | 0 | 0 | 0 |
| BRAMY2035719 | 0 | 0 | 0 | 15.497 | 56.534 | 0 | 0 |
| BRAMY2036076 | 10.816 | 0 | 0 | 0 | 0 | 0 | 11.995 |
| BRAMY2036459 | 1.885 | 0 | 0 | 1.923 | 0 | 0 | 0 |
| BRAMY2036567 | 7.474 | 17.148 | 0 | 7.626 | 0 | 0 | 0 |
| BRAMY2037823 | 29.664 | 0 | 0 | 0 | 0 | 0 | 0 |
| BRAMY2038516 | 0 | 87.230 | 0 | 0 | 0 | 0 | 0 |
| BRAMY2039630 | 0 | 0 | 30.744 | 0 | 0 | 0 | 0 |
| BRAMY2039728 | 0 | 0 | 0 | 37.268 | 0 | 0 | 0 |
| BRAMY2040095 | 1.709 | 0.784 | 1.211 | 2.092 | 0 | 0 | 1.516 |
| BRAMY2040214 | 0 | 0 | 0 | 44.393 | 0 | 0 | 16.084 |
| BRAMY2040592 | 3.482 | 0 | 0 | 7.105 | 0 | 0 | 0 |
| BRAMY2042195 | 0 | 69.484 | 0 | 0 | 0 | 0 | 0 |
| BRAMY2044838 | 0 | 0 | 0 | 0 | 0 | 0 | 68.762 |
| BRAMY2046871 | 33.165 | 0 | 0 | 0 | 0 | 0 | 0 |
| BRAMY2047420 | 0 | 0 | 0 | 0 | 3.770 | 0 | 0 |
| BRAMY3002329 | 20.323 | 0 | 0 | 34.558 | 0 | 0 | 15.025 |
| BRAMY3002458 | 0 | 0 | 0 | 50.663 | 0 | 0 | 0 |
| BRAMY3002803 | 14.428 | 0 | 0 | 44.161 | 0 | 0 | 0 |
| BRAMY3002991 | 0 | 8.995 | 0 | 0 | 0 | 0 | 4.348 |
| BRAMY3004126 | 49.811 | 0 | 0 | 0 | 0 | 0 | 0 |
| BRAMY3004224 | 33.027 | 0 | 0 | 33.695 | 0 | 0 | 0 |
| BRAMY3004377 | 3.317 | 0 | 0 | 0.846 | 0 | 2.990 | 0.920 |
| BRAMY3004800 | 38.061 | 0 | 0 | 46.598 | 0 | 0 | 0 |
| BRAMY3005091 | 19.193 | 0 | 0 | 0 | 0 | 0 | 0 |
| BRAMY3005184 | 0 | 0 | 0 | 22.861 | 0 | 0 | 0 |
| BRAMY3005912 | 21.416 | 0 | 0 | 0 | 0 | 0 | 0 |
| BRAMY3006761 | 32.935 | 0 | 0 | 56.003 | 0 | 0 | 0 |
| BRAMY3007078 | 0 | 0 | 0 | 50.312 | 0 | 0 | 0 |
| BRAMY3007311 | 23.569 | 18.024 | 0 | 0 | 0 | 0 | 0 |
| BRAMY3008044 | 7.175 | 8.231 | 6.355 | 3.660 | 0 | 0 | 3.979 |
| BRAMY3009556 | 0 | 0 | 0 | 0 | 0 | 0 | 52.395 |
| BRAMY3009782 | 0 | 0 | 0 | 0 | 0 | 0 | 52.395 |
| BRAMY3009811 | 0 | 0 | 0 | 66.943 | 0 | 0 | 0 |
| BRAMY3010008 | 0 | 0 | 2.062 | 0 | 0 | 0 | 0 |
| BRAMY3010264 | 49.811 | 0 | 0 | 0 | 0 | 0 | 0 |
| BRAMY3010492 | 5.759 | 0 | 0 | 0 | 21.436 | 0 | 6.387 |
| BRAMY3010654 | 0 | 0 | 0 | 0 | 0 | 0 | 52.395 |
| BRAMY4000089 | 5.089 | 0 | 0 | 15.576 | 0 | 0 | 0 |
| BRAMY4000095 | 0 | 33.520 | 51.759 | 0 | 0 | 0 | 0 |
| BRAMY4000229 | 0 | 0 | 0 | 0 | 0 | 0 | 39.237 |
| BRAMY4000915 | 0 | 0 | 0 | 50.312 | 0 | 0 | 0 |
| BRAMY4001863 | 0 | 0 | 0 | 0 | 0 | 0 | 16.612 |
| BRASW1000125 | 0 | 0 | 0 | 0 | 0 | 0.946 | 0 |
| BRAWH1000001 | 16.638 | 7.634 | 11.788 | 13.580 | 12.385 | 0 | 18.451 |
| BRAWH1000002 | 100 | 0 | 0 | 0 | 0 | 0 | 0 |
| BRAWH1000007 | 8.986 | 0 | 0 | 0 | 0 | 0 | 0 |
| BRAWH1000037 | 5.470 | 12.550 | 6.460 | 1.860 | 13.574 | 0 | 6.066 |
| BRAWH1000040 | 1.376 | 3.157 | 0 | 2.106 | 0 | 0 | 0.763 |
| BRAWH1000045 | 13.013 | 2.297 | 14.184 | 10.213 | 0 | 7.219 | 2.220 |
| BRAWH1000083 | 4.531 | 0 | 0 | 0 | 0 | 0 | 0 |
| BRAWH1000093 | 0.461 | 0 | 0 | 0 | 1.717 | 0 | 0.511 |
| BRAWH1000094 | 8.420 | 0 | 0 | 0 | 31.340 | 15.181 | 14.007 |
| BRAWH1000116 | 100 | 0 | 0 | 0 | 0 | 0 | 0 |
| BRAWH1000127 | 15.983 | 0 | 0 | 3.261 | 35.693 | 0 | 7.090 |
| BRAWH1000130 | 100 | 0 | 0 | 0 | 0 | 0 | 0 |
| BRAWH1000157 | 100 | 0 | 0 | 0 | 0 | 0 | 0 |
| BRAWH1000162 | 5.475 | 0 | 6.465 | 1.862 | 20.378 | 0 | 12.143 |
| BRAWH1000164 | 100 | 0 | 0 | 0 | 0 | 0 | 0 |
| BRAWH1000168 | 100 | 0 | 0 | 0 | 0 | 0 | 0 |
| BRAWH1000174 | 17.190 | 19.718 | 10.149 | 14.615 | 0 | 0 | 19.063 |
| BRAWH1000180 | 100 | 0 | 0 | 0 | 0 | 0 | 0 |
| BRAWH1000369 | 100 | 0 | 0 | 0 | 0 | 0 | 0 |
| BRAWH2000034 | 100 | 0 | 0 | 0 | 0 | 0 | 0 |
| BRAWH2000048 | 100 | 0 | 0 | 0 | 0 | 0 | 0 |
| BRAWH2000071 | 100 | 0 | 0 | 0 | 0 | 0 | 0 |
| BRAWH2000082 | 9.973 | 3.520 | 13.589 | 9.001 | 8.566 | 4.149 | 9.784 |
| BRAWH2000093 | 100 | 0 | 0 | 0 | 0 | 0 | 0 |
| BRAWH2000109 | 100 | 0 | 0 | 0 | 0 | 0 | 0 |
| BRAWH2000131 | 21.676 | 0 | 0 | 0 | 0 | 0 | 0 |
| BRAWH2000142 | 30.475 | 0 | 53.980 | 15.546 | 0 | 0 | 0 |
| BRAWH2000169 | 10.541 | 0 | 0 | 0 | 0 | 0 | 0 |
| BRAWH2000177 | 15.991 | 0 | 0 | 8.157 | 0 | 0 | 0 |
| BRAWH2000218 | 47.417 | 0 | 0 | 0 | 0 | 0 | 52.583 |
| BRAWH2000232 | 100 | 0 | 0 | 0 | 0 | 0 | 0 |
| BRAWH2000248 | 100 | 0 | 0 | 0 | 0 | 0 | 0 |
| BRAWH2000256 | 100 | 0 | 0 | 0 | 0 | 0 | 0 |
| BRAWH2000274 | 7.896 | 4.529 | 6.993 | 2.014 | 0 | 0 | 2.189 |
| BRAWH2000323 | 100 | 0 | 0 | 0 | 0 | 0 | 0 |
| BRAWH2000353 | 58.488 | 0 | 0 | 0 | 0 | 0 | 0 |
| BRAWH2000370 | 100 | 0 | 0 | 0 | 0 | 0 | 0 |
| BRAWH2000409 | 100 | 0 | 0 | 0 | 0 | 0 | 0 |
| BRAWH2000417 | 7.414 | 11.340 | 4.378 | 2.521 | 4.599 | 0 | 0 |
| BRAWH2000443 | 5.666 | 12.999 | 20.071 | 17.341 | 21.088 | 0 | 0 |
| BRAWH2000460 | 1.831 | 1.400 | 2.162 | 1.245 | 4.543 | 0 | 4.061 |
| BRAWH2000471 | 100 | 0 | 0 | 0 | 0 | 0 | 0 |
| BRAWH2000476 | 24.100 | 0 | 0 | 49.175 | 0 | 0 | 26.726 |
| BRAWH2000482 | 1.518 | 38.304 | 48.392 | 1.549 | 5.649 | 0 | 0 |
| BRAWH2000488 | 10.082 | 0 | 0 | 0 | 0 | 0 | 0 |
| BRAWH2000494 | 18.182 | 41.714 | 0 | 0 | 0 | 0 | 0 |
| BRAWH2000500 | 58.488 | 0 | 0 | 0 | 0 | 0 | 0 |
| BRAWH2000503 | 20.746 | 0 | 0 | 42.332 | 0 | 0 | 0 |
| BRAWH2000522 | 1.813 | 4.158 | 0 | 3.698 | 6.746 | 0 | 0 |
| BRAWH2000554 | 100 | 0 | 0 | 0 | 0 | 0 | 0 |
| BRAWH2000588 | 6.273 | 0 | 0 | 2.560 | 0 | 0 | 2.783 |
| BRAWH2000595 | 12.470 | 0 | 0 | 0 | 0 | 0 | 0 |
| BRAWH2000633 | 1.326 | 0 | 0 | 2.705 | 0 | 0 | 0 |
| BRAWH2000636 | 13.739 | 0 | 1.872 | 4.852 | 1.967 | 0 | 14.650 |
| BRAWH2000643 | 100 | 0 | 0 | 0 | 0 | 0 | 0 |
| BRAWH2000651 | 100 | 0 | 0 | 0 | 0 | 0 | 0 |
| BRAWH2000682 | 100 | 0 | 0 | 0 | 0 | 0 | 0 |
| BRAWH2000686 | 1.222 | 5.609 | 8.661 | 1.247 | 4.550 | 4.407 | 6.778 |
| BRAWH2000697 | 3.035 | 2.321 | 10.750 | 4.128 | 7.530 | 7.295 | 5.609 |
| BRAWH2000752 | 100 | 0 | 0 | 0 | 0 | 0 | 0 |
| BRAWH2000839 | 6.982 | 3.769 | 2.910 | 4.609 | 3.057 | 1.481 | 5.465 |
| BRAWH2000859 | 47.167 | 0 | 0 | 0 | 0 | 0 | 0 |
| BRAWH2000866 | 100 | 0 | 0 | 0 | 0 | 0 | 0 |
| BRAWH2000892 | 100 | 0 | 0 | 0 | 0 | 0 | 0 |
| BRAWH2000901 | 100 | 0 | 0 | 0 | 0 | 0 | 0 |
| BRAWH2000924 | 8.897 | 4.609 | 9.150 | 7.320 | 10.682 | 10.348 | 6.047 |
| BRAWH2000926 | 4.218 | 0 | 0 | 0 | 0 | 0 | 0 |
| BRAWH2000944 | 5.217 | 0 | 0 | 0 | 0 | 0 | 0 |
| BRAWH2000984 | 100 | 0 | 0 | 0 | 0 | 0 | 0 |
| BRAWH2001019 | 2.281 | 15.697 | 16.159 | 9.307 | 16.977 | 0 | 2.529 |
| BRAWH2001037 | 4.010 | 0 | 0 | 0 | 0 | 0 | 0 |
| BRAWH2001052 | 100 | 0 | 0 | 0 | 0 | 0 | 0 |
| BRAWH2001092 | 2.109 | 0.403 | 0.623 | 1.434 | 0 | 0 | 0.975 |
| BRAWH2001103 | 13.143 | 0 | 0 | 6.704 | 0 | 0 | 0 |
| BRAWH2001129 | 47.167 | 0 | 0 | 0 | 0 | 0 | 0 |
| BRAWH2001137 | 100 | 0 | 0 | 0 | 0 | 0 | 0 |
| BRAWH2001141 | 5.082 | 0 | 0 | 0 | 0 | 0 | 0 |
| BRAWH2001159 | 100 | 0 | 0 | 0 | 0 | 0 | 0 |
| BRAWH2001166 | 3.157 | 0 | 0 | 0 | 0 | 0 | 3.501 |
| BRAWH2001171 | 6.176 | 1.417 | 0 | 1.890 | 2.299 | 0 | 4.794 |
| BRAWH2001186 | 2.849 | 0 | 0 | 0 | 0 | 0 | 0 |
| BRAWH2001203 | 100 | 0 | 0 | 0 | 0 | 0 | 0 |
| BRAWH2001238 | 12.091 | 0 | 0 | 0 | 45.001 | 0 | 0 |
| BRAWH2001239 | 7.296 | 0 | 0 | 0 | 27.156 | 0 | 8.091 |
| BRAWH2001255 | 100 | 0 | 0 | 0 | 0 | 0 | 0 |
| BRAWH2001321 | 6.868 | 1.969 | 3.041 | 10.510 | 3.195 | 3.095 | 6.664 |
| BRAWH2001355 | 100 | 0 | 0 | 0 | 0 | 0 | 0 |
| BRAWH2001378 | 100 | 0 | 0 | 0 | 0 | 0 | 0 |
| BRAWH2001395 | 14.290 | 5.785 | 10.422 | 22.297 | 12.514 | 4.546 | 11.652 |
| BRAWH2001406 | 3.385 | 0 | 0 | 3.454 | 12.600 | 0 | 0 |
| BRAWH2001412 | 15.036 | 0 | 0 | 0 | 0 | 0 | 0 |
| BRAWH2001418 | 5.158 | 17.751 | 18.274 | 2.631 | 9.600 | 18.599 | 2.860 |
| BRAWH2001424 | 5.091 | 0 | 0 | 0 | 0 | 0 | 5.646 |
| BRAWH2001436 | 100 | 0 | 0 | 0 | 0 | 0 | 0 |
| BRAWH2001438 | 100 | 0 | 0 | 0 | 0 | 0 | 0 |
| BRAWH2001439 | 4.116 | 0 | 7.290 | 9.448 | 0 | 0 | 3.423 |
| BRAWH2001459 | 4.891 | 0 | 8.664 | 2.495 | 9.103 | 0 | 51.532 |
| BRAWH2001461 | 3.555 | 0 | 0 | 0 | 0 | 0 | 3.942 |
| BRAWH2001484 | 10.625 | 0 | 37.639 | 0 | 0 | 0 | 0 |
| BRAWH2001492 | 100 | 0 | 0 | 0 | 0 | 0 | 0 |
| BRAWH2001503 | 100 | 0 | 0 | 0 | 0 | 0 | 0 |
| BRAWH2001519 | 100 | 0 | 0 | 0 | 0 | 0 | 0 |
| BRAWH2001530 | 7.633 | 17.512 | 0 | 0 | 56.821 | 0 | 0 |
| BRAWH2001535 | 1.975 | 15.859 | 5.247 | 10.075 | 16.539 | 14.243 | 10.951 |
| BRAWH2001589 | 100 | 0 | 0 | 0 | 0 | 0 | 0 |
| BRAWH2001662 | 4.598 | 0 | 16.288 | 0 | 0 | 0 | 5.099 |
| BRAWH2001666 | 100 | 0 | 0 | 0 | 0 | 0 | 0 |
| BRAWH2001671 | 7.605 | 0 | 0 | 11.638 | 0 | 0 | 12.650 |
| BRAWH2001679 | 100 | 0 | 0 | 0 | 0 | 0 | 0 |
| BRAWH2001686 | 100 | 0 | 0 | 0 | 0 | 0 | 0 |
| BRAWH2001701 | 100 | 0 | 0 | 0 | 0 | 0 | 0 |
| BRAWH2001842 | 100 | 0 | 0 | 0 | 0 | 0 | 0 |
| BRAWH2001862 | 3.358 | 0 | 0 | 0 | 0 | 0 | 0 |
| BRAWH2001873 | 100 | 0 | 0 | 0 | 0 | 0 | 0 |
| BRAWH2001940 | 37.398 | 0 | 0 | 38.155 | 0 | 0 | 0 |
| BRAWH2001973 | 37.398 | 0 | 0 | 38.155 | 0 | 0 | 0 |
| BRAWH2002047 | 58.488 | 0 | 0 | 0 | 0 | 0 | 0 |
| BRAWH2002060 | 58.488 | 0 | 0 | 0 | 0 | 0 | 0 |
| BRAWH2002062 | 54.530 | 0 | 0 | 0 | 0 | 0 | 0 |
| BRAWH2002125 | 100 | 0 | 0 | 0 | 0 | 0 | 0 |
| BRAWH2002191 | 100 | 0 | 0 | 0 | 0 | 0 | 0 |
| BRAWH2002318 | 26.686 | 0 | 0 | 0 | 0 | 0 | 0 |
| BRAWH2002333 | 100 | 0 | 0 | 0 | 0 | 0 | 0 |
| BRAWH2002352 | 100 | 0 | 0 | 0 | 0 | 0 | 0 |
| BRAWH2002383 | 100 | 0 | 0 | 0 | 0 | 0 | 0 |
| BRAWH2002549 | 18.017 | 0 | 63.828 | 0 | 0 | 0 | 0 |
| BRAWH2002560 | 6.454 | 0 | 0 | 3.292 | 12.010 | 11.635 | 0 |
| BRAWH2002601 | 4.974 | 11.411 | 17.620 | 5.074 | 0 | 17.934 | 0 |
| BRAWH2002623 | 100 | 0 | 0 | 0 | 0 | 0 | 0 |
| BRAWH2002630 | 100 | 0 | 0 | 0 | 0 | 0 | 0 |
| BRAWH2002635 | 100 | 0 | 0 | 0 | 0 | 0 | 0 |
| BRAWH2002662 | 100 | 0 | 0 | 0 | 0 | 0 | 0 |
| BRAWH2002679 | 9.467 | 21.720 | 0 | 0 | 35.237 | 0 | 0 |
| BRAWH2002725 | 100 | 0 | 0 | 0 | 0 | 0 | 0 |
| BRAWH2002761 | 100 | 0 | 0 | 0 | 0 | 0 | 0 |
| BRAWH2002811 | 8.624 | 0 | 0 | 17.598 | 0 | 0 | 9.564 |
| BRAWH2002861 | 100 | 0 | 0 | 0 | 0 | 0 | 0 |
| BRAWH2002911 | 23.795 | 0 | 0 | 0 | 0 | 0 | 0 |
| BRAWH2002963 | 11.932 | 0 | 0 | 0 | 0 | 0 | 0 |
| BRAWH2002967 | 44.597 | 0 | 0 | 0 | 0 | 0 | 0 |
| BRAWH2002976 | 100 | 0 | 0 | 0 | 0 | 0 | 0 |
| BRAWH2003000 | 58.488 | 0 | 0 | 0 | 0 | 0 | 0 |
| BRAWH2003025 | 6.064 | 0 | 0 | 0 | 11.285 | 0 | 0 |
| BRAWH2003135 | 34.934 | 0 | 0 | 0 | 0 | 0 | 0 |
| BRAWH2003240 | 7.848 | 0 | 0 | 8.007 | 0 | 0 | 0 |
| BRAWH2003307 | 100 | 0 | 0 | 0 | 0 | 0 | 0 |
| BRAWH2003355 | 21.676 | 0 | 0 | 0 | 0 | 0 | 0 |
| BRAWH2003366 | 100 | 0 | 0 | 0 | 0 | 0 | 0 |
| BRAWH2003662 | 12.395 | 0 | 21.955 | 18.969 | 0 | 0 | 0 |
| BRAWH2003689 | 47.167 | 0 | 0 | 0 | 0 | 0 | 0 |
| BRAWH2003693 | 13.685 | 0 | 0 | 6.981 | 12.734 | 0 | 3.794 |
| BRAWH2003818 | 3.027 | 0 | 0 | 0 | 0 | 0 | 0 |
| BRAWH2003832 | 100 | 0 | 0 | 0 | 0 | 0 | 0 |
| BRAWH2003948 | 100 | 0 | 0 | 0 | 0 | 0 | 0 |
| BRAWH2003964 | 49.499 | 0 | 0 | 50.501 | 0 | 0 | 0 |
| BRAWH2004044 | 100 | 0 | 0 | 0 | 0 | 0 | 0 |
| BRAWH2004068 | 100 | 0 | 0 | 0 | 0 | 0 | 0 |
| BRAWH2004078 | 10.539 | 0 | 0 | 0 | 0 | 0 | 0 |
| BRAWH2004095 | 11.821 | 0 | 0 | 4.824 | 0 | 0 | 5.244 |
| BRAWH2004136 | 15.911 | 36.504 | 0 | 0 | 0 | 0 | 0 |
| BRAWH2004217 | 54.530 | 0 | 0 | 0 | 0 | 0 | 0 |
| BRAWH2004580 | 18.183 | 0 | 0 | 18.551 | 0 | 0 | 0 |
| BRAWH2004731 | 100 | 0 | 0 | 0 | 0 | 0 | 0 |
| BRAWH2004779 | 49.811 | 0 | 0 | 0 | 0 | 0 | 0 |
| BRAWH2004842 | 43.845 | 9.145 | 0 | 4.067 | 0 | 0 | 8.840 |
| BRAWH2004884 | 100 | 0 | 0 | 0 | 0 | 0 | 0 |
| BRAWH2004923 | 100 | 0 | 0 | 0 | 0 | 0 | 0 |
| BRAWH2005008 | 16.511 | 0 | 0 | 16.845 | 0 | 0 | 0 |
| BRAWH2005068 | 0.402 | 0 | 0 | 0 | 0 | 4.351 | 0 |
| BRAWH2005074 | 35.119 | 0 | 0 | 0 | 0 | 0 | 0 |
| BRAWH2005225 | 5.797 | 0 | 0 | 0 | 0 | 0 | 0 |
| BRAWH2005315 | 100 | 0 | 0 | 0 | 0 | 0 | 0 |
| BRAWH2005517 | 44.597 | 0 | 0 | 0 | 0 | 0 | 0 |
| BRAWH2005524 | 100 | 0 | 0 | 0 | 0 | 0 | 0 |
| BRAWH2005533 | 100 | 0 | 0 | 0 | 0 | 0 | 0 |
| BRAWH2005578 | 26.802 | 0 | 0 | 27.344 | 0 | 0 | 14.861 |
| BRAWH2005661 | 3.197 | 3.668 | 0 | 6.524 | 0 | 0 | 3.546 |
| BRAWH2005812 | 100 | 0 | 0 | 0 | 0 | 0 | 0 |
| BRAWH2005974 | 100 | 0 | 0 | 0 | 0 | 0 | 0 |
| BRAWH2005998 | 30.356 | 69.644 | 0 | 0 | 0 | 0 | 0 |
| BRAWH2006044 | 17.594 | 0 | 0 | 0 | 0 | 0 | 0 |
| BRAWH2006083 | 100 | 0 | 0 | 0 | 0 | 0 | 0 |
| BRAWH2006207 | 12.943 | 29.695 | 0 | 0 | 48.175 | 0 | 0 |
| BRAWH2006301 | 58.488 | 0 | 0 | 0 | 0 | 0 | 0 |
| BRAWH2006395 | 12.446 | 0 | 0 | 0 | 0 | 0 | 0 |
| BRAWH2006405 | 12.977 | 0 | 0 | 13.240 | 0 | 0 | 0 |
| BRAWH2006450 | 20.085 | 11.520 | 0 | 0 | 0 | 0 | 22.274 |
| BRAWH2006493 | 5.086 | 11.669 | 0 | 0 | 18.931 | 0 | 22.562 |
| BRAWH2006526 | 54.530 | 0 | 0 | 0 | 0 | 0 | 0 |
| BRAWH2006543 | 30.705 | 10.063 | 0 | 0 | 0 | 0 | 24.322 |
| BRAWH2006622 | 16.546 | 0 | 0 | 0 | 0 | 0 | 0 |
| BRAWH2006760 | 100 | 0 | 0 | 0 | 0 | 0 | 0 |
| BRAWH2006960 | 100 | 0 | 0 | 0 | 0 | 0 | 0 |
| BRAWH2006989 | 24.975 | 57.298 | 0 | 0 | 0 | 0 | 0 |
| BRAWH2006996 | 10.204 | 0 | 0 | 0 | 0 | 0 | 3.772 |
| BRAWH2007069 | 100 | 0 | 0 | 0 | 0 | 0 | 0 |
| BRAWH2007308 | 100 | 0 | 0 | 0 | 0 | 0 | 0 |
| BRAWH2007406 | 100 | 0 | 0 | 0 | 0 | 0 | 0 |
| BRAWH2007550 | 100 | 0 | 0 | 0 | 0 | 0 | 0 |
| BRAWH2007570 | 100 | 0 | 0 | 0 | 0 | 0 | 0 |
| BRAWH2007591 | 40.796 | 0 | 11.117 | 16.008 | 0 | 0 | 3.480 |
| BRAWH2007605 | 6.154 | 0 | 0 | 6.278 | 0 | 0 | 9.099 |
| BRAWH2007658 | 58.101 | 0 | 0 | 0 | 0 | 41.899 | 0 |
| BRAWH2007664 | 10.232 | 23.474 | 0 | 0 | 0 | 0 | 0 |
| BRAWH2007800 | 2.349 | 0 | 0 | 0 | 0 | 0 | 2.605 |
| BRAWH2007808 | 37.485 | 0 | 0 | 0 | 0 | 0 | 0 |
| BRAWH2007825 | 58.488 | 0 | 0 | 0 | 0 | 0 | 0 |
| BRAWH2007862 | 100 | 0 | 0 | 0 | 0 | 0 | 0 |
| BRAWH2007890 | 100 | 0 | 0 | 0 | 0 | 0 | 0 |
| BRAWH2008058 | 23.426 | 0 | 0 | 0 | 0 | 0 | 0 |
| BRAWH2008128 | 22.081 | 0 | 0 | 0 | 0 | 0 | 0 |
| BRAWH2008219 | 14.410 | 0 | 0 | 0 | 0 | 0 | 0 |
| BRAWH2008255 | 4.225 | 0 | 0 | 0 | 0 | 0 | 0 |
| BRAWH2008292 | 36.501 | 0 | 0 | 0 | 0 | 0 | 0 |
| BRAWH2008297 | 100 | 0 | 0 | 0 | 0 | 0 | 0 |
| BRAWH2008366 | 12.752 | 0 | 0 | 0 | 0 | 0 | 0 |
| BRAWH2008380 | 100 | 0 | 0 | 0 | 0 | 0 | 0 |
| BRAWH2008540 | 54.530 | 0 | 0 | 0 | 0 | 0 | 0 |
| BRAWH2008577 | 3.555 | 0 | 0 | 0 | 0 | 0 | 0 |
| BRAWH2008706 | 28.755 | 0 | 0 | 14.668 | 0 | 0 | 3.189 |
| BRAWH2008790 | 100 | 0 | 0 | 0 | 0 | 0 | 0 |
| BRAWH2008903 | 16.913 | 9.401 | 0 | 2.941 | 0 | 0 | 28.133 |
| BRAWH2008956 | 15.570 | 0 | 0 | 15.885 | 0 | 0 | 0 |
| BRAWH2008993 | 49.811 | 0 | 0 | 0 | 0 | 0 | 0 |
| BRAWH2009112 | 100 | 0 | 0 | 0 | 0 | 0 | 0 |
| BRAWH2009227 | 15.805 | 0 | 0 | 0 | 0 | 0 | 0 |
| BRAWH2009238 | 26.065 | 0 | 0 | 21.274 | 0 | 0 | 17.343 |
| BRAWH2009261 | 100 | 0 | 0 | 0 | 0 | 0 | 0 |
| BRAWH2009263 | 100 | 0 | 0 | 0 | 0 | 0 | 0 |
| BRAWH2009298 | 100 | 0 | 0 | 0 | 0 | 0 | 0 |
| BRAWH2009304 | 3.922 | 0 | 13.895 | 0 | 0 | 0 | 8.699 |
| BRAWH2009307 | 3.982 | 0 | 0 | 2.031 | 0 | 0 | 4.416 |
| BRAWH2009360 | 8.653 | 0 | 30.656 | 0 | 0 | 0 | 0 |
| BRAWH2009393 | 100 | 0 | 0 | 0 | 0 | 0 | 0 |
| BRAWH2009485 | 100 | 0 | 0 | 0 | 0 | 0 | 0 |
| BRAWH2009490 | 3.383 | 0 | 0 | 0 | 0 | 12.199 | 0 |
| BRAWH2009558 | 100 | 0 | 0 | 0 | 0 | 0 | 0 |
| BRAWH2009590 | 100 | 0 | 0 | 0 | 0 | 0 | 0 |
| BRAWH2009626 | 44.597 | 0 | 0 | 0 | 0 | 0 | 0 |
| BRAWH2009678 | 21.269 | 0 | 0 | 0 | 0 | 0 | 0 |
| BRAWH2009695 | 26.755 | 0 | 0 | 27.297 | 0 | 0 | 0 |
| BRAWH2009907 | 100 | 0 | 0 | 0 | 0 | 0 | 0 |
| BRAWH2010000 | 18.745 | 43.006 | 0 | 38.249 | 0 | 0 | 0 |
| BRAWH2010069 | 100 | 0 | 0 | 0 | 0 | 0 | 0 |
| BRAWH2010084 | 100 | 0 | 0 | 0 | 0 | 0 | 0 |
| BRAWH2010136 | 26.404 | 0 | 0 | 0 | 0 | 0 | 0 |
| BRAWH2010318 | 100 | 0 | 0 | 0 | 0 | 0 | 0 |
| BRAWH2010329 | 20.017 | 0 | 0 | 15.317 | 0 | 0 | 11.099 |
| BRAWH2010354 | 12.977 | 29.773 | 0 | 0 | 0 | 0 | 0 |
| BRAWH2010364 | 34.464 | 0 | 0 | 17.581 | 0 | 0 | 0 |
| BRAWH2010536 | 14.718 | 0 | 0 | 0 | 0 | 0 | 0 |
| BRAWH2010552 | 58.488 | 0 | 0 | 0 | 0 | 0 | 0 |
| BRAWH2010584 | 30.356 | 69.644 | 0 | 0 | 0 | 0 | 0 |
| BRAWH2010618 | 100 | 0 | 0 | 0 | 0 | 0 | 0 |
| BRAWH2010619 | 100 | 0 | 0 | 0 | 0 | 0 | 0 |
| BRAWH2010754 | 11.186 | 0 | 0 | 0 | 0 | 40.334 | 0 |
| BRAWH2010980 | 7.066 | 0 | 0 | 0 | 0 | 0 | 0 |
| BRAWH2011066 | 21.178 | 0 | 0 | 0 | 78.822 | 0 | 0 |
| BRAWH2011079 | 100 | 0 | 0 | 0 | 0 | 0 | 0 |
| BRAWH2011096 | 54.530 | 0 | 0 | 0 | 0 | 0 | 0 |
| BRAWH2011155 | 100 | 0 | 0 | 0 | 0 | 0 | 0 |
| BRAWH2011166 | 100 | 0 | 0 | 0 | 0 | 0 | 0 |
| BRAWH2011204 | 49.811 | 0 | 0 | 0 | 0 | 0 | 0 |
| BRAWH2011286 | 8.924 | 0 | 0 | 0 | 0 | 0 | 0 |
| BRAWH2011294 | 100 | 0 | 0 | 0 | 0 | 0 | 0 |
| BRAWH2011343 | 7.015 | 0 | 0 | 7.157 | 0 | 0 | 7.779 |
| BRAWH2011400 | 100 | 0 | 0 | 0 | 0 | 0 | 0 |
| BRAWH2011762 | 100 | 0 | 0 | 0 | 0 | 0 | 0 |
| BRAWH2011795 | 100 | 0 | 0 | 0 | 0 | 0 | 0 |
| BRAWH2011796 | 100 | 0 | 0 | 0 | 0 | 0 | 0 |
| BRAWH2011812 | 13.544 | 0 | 0 | 0 | 0 | 0 | 0 |
| BRAWH2011823 | 21.626 | 0 | 0 | 0 | 0 | 0 | 0 |
| BRAWH2011860 | 100 | 0 | 0 | 0 | 0 | 0 | 0 |
| BRAWH2011920 | 18.948 | 0 | 0 | 19.331 | 0 | 0 | 0 |
| BRAWH2011958 | 100 | 0 | 0 | 0 | 0 | 0 | 0 |
| BRAWH2012054 | 100 | 0 | 0 | 0 | 0 | 0 | 0 |
| BRAWH2012077 | 12.529 | 0 | 0 | 0 | 0 | 0 | 0 |
| BRAWH2012161 | 3.850 | 0 | 0 | 0 | 0 | 0 | 0 |
| BRAWH2012162 | 36.060 | 0 | 0 | 0 | 0 | 0 | 0 |
| BRAWH2012164 | 20.835 | 0 | 0 | 0 | 0 | 0 | 0 |
| BRAWH2012258 | 100 | 0 | 0 | 0 | 0 | 0 | 0 |
| BRAWH2012277 | 28.630 | 0 | 0 | 0 | 0 | 0 | 0 |
| BRAWH2012326 | 100 | 0 | 0 | 0 | 0 | 0 | 0 |
| BRAWH2012538 | 10.145 | 0 | 0 | 0 | 18.880 | 0 | 0 |
| BRAWH2012540 | 100 | 0 | 0 | 0 | 0 | 0 | 0 |
| BRAWH2012619 | 100 | 0 | 0 | 0 | 0 | 0 | 0 |
| BRAWH2012698 | 19.174 | 0 | 0 | 0 | 0 | 0 | 0 |
| BRAWH2012749 | 100 | 0 | 0 | 0 | 0 | 0 | 0 |
| BRAWH2012827 | 8.993 | 0 | 31.859 | 0 | 33.472 | 0 | 0 |
| BRAWH2012866 | 8.503 | 0 | 0 | 4.337 | 0 | 7.665 | 11.786 |
| BRAWH2012963 | 100 | 0 | 0 | 0 | 0 | 0 | 0 |
| BRAWH2013047 | 18.786 | 0 | 0 | 0 | 0 | 0 | 0 |
| BRAWH2013219 | 19.375 | 0 | 0 | 0 | 0 | 0 | 0 |
| BRAWH2013294 | 39.442 | 0 | 0 | 10.060 | 0 | 0 | 0 |
| BRAWH2013748 | 2.669 | 6.123 | 9.455 | 2.723 | 9.934 | 0 | 2.960 |
| BRAWH2013866 | 100 | 0 | 0 | 0 | 0 | 0 | 0 |
| BRAWH2013871 | 37.485 | 0 | 0 | 0 | 0 | 0 | 0 |
| BRAWH2013914 | 47.417 | 0 | 0 | 0 | 0 | 0 | 52.583 |
| BRAWH2013941 | 100 | 0 | 0 | 0 | 0 | 0 | 0 |
| BRAWH2013955 | 100 | 0 | 0 | 0 | 0 | 0 | 0 |
| BRAWH2014053 | 23.222 | 0 | 0 | 0 | 0 | 0 | 25.752 |
| BRAWH2014188 | 2.148 | 0 | 0 | 3.652 | 0 | 0 | 0.794 |
| BRAWH2014234 | 100 | 0 | 0 | 0 | 0 | 0 | 0 |
| BRAWH2014379 | 62.276 | 0 | 0 | 0 | 0 | 0 | 0 |
| BRAWH2014414 | 17.865 | 0 | 0 | 18.227 | 0 | 0 | 9.906 |
| BRAWH2014473 | 45.606 | 0 | 0 | 0 | 0 | 0 | 0 |
| BRAWH2014511 | 4.297 | 9.858 | 30.443 | 0 | 0 | 15.493 | 0 |
| BRAWH2014645 | 4.228 | 0 | 3.745 | 1.078 | 0 | 0 | 1.172 |
| BRAWH2014662 | 15.521 | 0 | 0 | 0 | 0 | 0 | 17.212 |
| BRAWH2014717 | 100 | 0 | 0 | 0 | 0 | 0 | 0 |
| BRAWH2014729 | 100 | 0 | 0 | 0 | 0 | 0 | 0 |
| BRAWH2014876 | 10.473 | 24.026 | 0 | 0 | 0 | 0 | 58.068 |
| BRAWH2014934 | 35.975 | 0 | 0 | 0 | 0 | 0 | 0 |
| BRAWH2014954 | 58.488 | 0 | 0 | 0 | 0 | 0 | 0 |
| BRAWH2015247 | 10.440 | 0 | 0 | 0 | 0 | 0 | 11.577 |
| BRAWH2015349 | 21.997 | 0 | 0 | 0 | 0 | 39.658 | 0 |
| BRAWH2015375 | 100 | 0 | 0 | 0 | 0 | 0 | 0 |
| BRAWH2015385 | 58.488 | 0 | 0 | 0 | 0 | 0 | 0 |
| BRAWH2015595 | 100 | 0 | 0 | 0 | 0 | 0 | 0 |
| BRAWH2015728 | 49.811 | 0 | 0 | 0 | 0 | 0 | 0 |
| BRAWH2015853 | 100 | 0 | 0 | 0 | 0 | 0 | 0 |
| BRAWH2015866 | 100 | 0 | 0 | 0 | 0 | 0 | 0 |
| BRAWH2016028 | 49.811 | 0 | 0 | 0 | 0 | 0 | 0 |
| BRAWH2016106 | 36.501 | 0 | 0 | 0 | 0 | 0 | 0 |
| BRAWH2016160 | 100 | 0 | 0 | 0 | 0 | 0 | 0 |
| BRAWH2016166 | 22.688 | 0 | 0 | 7.716 | 0 | 0 | 0 |
| BRAWH2016209 | 8.306 | 0 | 0 | 0 | 0 | 0 | 0 |
| BRAWH2016221 | 47.417 | 0 | 0 | 0 | 0 | 0 | 52.583 |
| BRAWH2016223 | 100 | 0 | 0 | 0 | 0 | 0 | 0 |
| BRAWH2016269 | 1.318 | 3.025 | 4.670 | 0 | 4.907 | 0 | 0 |
| BRAWH2016305 | 4.308 | 0 | 0 | 0 | 0 | 0 | 0 |
| BRAWH2016439 | 100 | 0 | 0 | 0 | 0 | 0 | 0 |
| BRAWH2016446 | 8.700 | 0 | 0 | 0 | 0 | 0 | 0 |
| BRAWH2016514 | 35.037 | 0 | 0 | 35.747 | 0 | 0 | 0 |
| BRAWH2016562 | 49.499 | 0 | 0 | 50.501 | 0 | 0 | 0 |
| BRAWH2016655 | 100 | 0 | 0 | 0 | 0 | 0 | 0 |
| BRAWH2016679 | 9.636 | 0 | 0 | 0 | 0 | 0 | 0 |
| BRAWH2016702 | 73.807 | 0 | 0 | 0 | 0 | 0 | 0 |
| BRAWH2016724 | 35.119 | 0 | 0 | 0 | 0 | 0 | 0 |
| BRAWH2016785 | 100 | 0 | 0 | 0 | 0 | 0 | 0 |
| BRAWH2016792 | 100 | 0 | 0 | 0 | 0 | 0 | 0 |
| BRAWH2016800 | 100 | 0 | 0 | 0 | 0 | 0 | 0 |
| BRAWH2017002 | 2.488 | 0 | 0 | 2.539 | 0 | 8.973 | 0 |
| BRAWH2017103 | 100 | 0 | 0 | 0 | 0 | 0 | 0 |
| BRAWH2017250 | 100 | 0 | 0 | 0 | 0 | 0 | 0 |
| BRAWH2017294 | 47.417 | 0 | 0 | 0 | 0 | 0 | 52.583 |
| BRAWH2017304 | 16.605 | 0 | 0 | 0 | 0 | 0 | 0 |
| BRAWH2017305 | 100 | 0 | 0 | 0 | 0 | 0 | 0 |
| BRAWH2017379 | 15.195 | 0 | 0 | 0 | 0 | 0 | 16.850 |
| BRAWH2017407 | 100 | 0 | 0 | 0 | 0 | 0 | 0 |
| BRAWH2017433 | 100 | 0 | 0 | 0 | 0 | 0 | 0 |
| BRAWH2017523 | 100 | 0 | 0 | 0 | 0 | 0 | 0 |
| BRAWH2017534 | 100 | 0 | 0 | 0 | 0 | 0 | 0 |
| BRAWH2017542 | 35.119 | 0 | 0 | 0 | 0 | 0 | 0 |
| BRAWH2017635 | 100 | 0 | 0 | 0 | 0 | 0 | 0 |
| BRAWH2017685 | 1.522 | 1.164 | 0 | 0.518 | 0 | 0 | 1.126 |
| BRAWH2017913 | 100 | 0 | 0 | 0 | 0 | 0 | 0 |
| BRAWH2018206 | 11.051 | 0 | 0 | 11.275 | 0 | 0 | 36.766 |
| BRAWH2018267 | 41.331 | 0 | 0 | 21.084 | 0 | 0 | 22.917 |
| BRAWH2018282 | 100 | 0 | 0 | 0 | 0 | 0 | 0 |
| BRAWH2018317 | 100 | 0 | 0 | 0 | 0 | 0 | 0 |
| BRAWH2018506 | 100 | 0 | 0 | 0 | 0 | 0 | 0 |
| BRAWH2018514 | 100 | 0 | 0 | 0 | 0 | 0 | 0 |
| BRAWH2018526 | 0.615 | 1.412 | 2.180 | 0.628 | 0 | 0 | 0.341 |
| BRAWH2018527 | 8.512 | 0 | 0 | 0 | 0 | 0 | 0 |
| BRAWH2018601 | 100 | 0 | 0 | 0 | 0 | 0 | 0 |
| BRAWH2018729 | 2.980 | 0 | 0 | 0 | 0 | 0 | 0 |
| BRAWH2018745 | 45.606 | 0 | 0 | 0 | 0 | 0 | 0 |
| BRAWH2018875 | 52.989 | 0 | 0 | 13.515 | 0 | 0 | 14.691 |
| BRAWH2019053 | 100 | 0 | 0 | 0 | 0 | 0 | 0 |
| BRAWH2019055 | 100 | 0 | 0 | 0 | 0 | 0 | 0 |
| BRAWH2019198 | 100 | 0 | 0 | 0 | 0 | 0 | 0 |
| BRAWH3000078 | 100 | 0 | 0 | 0 | 0 | 0 | 0 |
| BRAWH3000088 | 5.784 | 0 | 4.098 | 4.721 | 4.306 | 4.171 | 10.263 |
| BRAWH3000100 | 100 | 0 | 0 | 0 | 0 | 0 | 0 |
| BRAWH3000166 | 100 | 0 | 0 | 0 | 0 | 0 | 0 |
| BRAWH3000314 | 71.553 | 0 | 0 | 0 | 0 | 0 | 0 |
| BRAWH3000329 | 100 | 0 | 0 | 0 | 0 | 0 | 0 |
| BRAWH3000410 | 100 | 0 | 0 | 0 | 0 | 0 | 0 |
| BRAWH3000446 | 100 | 0 | 0 | 0 | 0 | 0 | 0 |
| BRAWH3000491 | 100 | 0 | 0 | 0 | 0 | 0 | 0 |
| BRAWH3000604 | 54.530 | 0 | 0 | 0 | 0 | 0 | 0 |
| BRAWH3000884 | 100 | 0 | 0 | 0 | 0 | 0 | 0 |
| BRAWH3001053 | 100 | 0 | 0 | 0 | 0 | 0 | 0 |
| BRAWH3001319 | 27.294 | 0 | 0 | 0 | 0 | 0 | 0 |
| BRAWH3001326 | 45.606 | 0 | 0 | 0 | 0 | 0 | 0 |
| BRAWH3001475 | 100 | 0 | 0 | 0 | 0 | 0 | 0 |
| BRAWH3001636 | 100 | 0 | 0 | 0 | 0 | 0 | 0 |
| BRAWH3001638 | 100 | 0 | 0 | 0 | 0 | 0 | 0 |
| BRAWH3001712 | 49.499 | 0 | 0 | 50.501 | 0 | 0 | 0 |
| BRAWH3001760 | 100 | 0 | 0 | 0 | 0 | 0 | 0 |
| BRAWH3001783 | 100 | 0 | 0 | 0 | 0 | 0 | 0 |
| BRAWH3001833 | 100 | 0 | 0 | 0 | 0 | 0 | 0 |
| BRAWH3001879 | 100 | 0 | 0 | 0 | 0 | 0 | 0 |
| BRAWH3001891 | 34.539 | 0 | 0 | 0 | 0 | 0 | 0 |
| BRAWH3001918 | 27.772 | 0 | 0 | 0 | 0 | 0 | 0 |
| BRAWH3002433 | 47.417 | 0 | 0 | 0 | 0 | 0 | 52.583 |
| BRAWH3002467 | 36.501 | 0 | 0 | 0 | 0 | 0 | 0 |
| BRAWH3002513 | 100 | 0 | 0 | 0 | 0 | 0 | 0 |
| BRAWH3002574 | 13.222 | 0 | 0 | 0 | 0 | 0 | 0 |
| BRAWH3002600 | 36.800 | 0 | 0 | 0 | 0 | 0 | 0 |
| BRAWH3002819 | 100 | 0 | 0 | 0 | 0 | 0 | 0 |
| BRAWH3002853 | 2.959 | 0 | 2.620 | 1.509 | 2.753 | 0 | 4.922 |
| BRAWH3003019 | 100 | 0 | 0 | 0 | 0 | 0 | 0 |
| BRAWH3003136 | 100 | 0 | 0 | 0 | 0 | 0 | 0 |
| BRAWH3003244 | 100 | 0 | 0 | 0 | 0 | 0 | 0 |
| BRAWH3003343 | 100 | 0 | 0 | 0 | 0 | 0 | 0 |
| BRAWH3003411 | 46.281 | 0 | 0 | 0 | 0 | 0 | 0 |
| BRAWH3003522 | 100 | 0 | 0 | 0 | 0 | 0 | 0 |
| BRAWH3003573 | 20.162 | 0 | 0 | 0 | 0 | 0 | 22.358 |
| BRAWH3003598 | 100 | 0 | 0 | 0 | 0 | 0 | 0 |
| BRAWH3003727 | 10.055 | 0 | 0 | 10.259 | 0 | 0 | 0 |
| BRAWH3003801 | 100 | 0 | 0 | 0 | 0 | 0 | 0 |
| BRAWH3003975 | 100 | 0 | 0 | 0 | 0 | 0 | 0 |
| BRAWH3004222 | 44.597 | 0 | 0 | 0 | 0 | 0 | 0 |
| BRAWH3004335 | 100 | 0 | 0 | 0 | 0 | 0 | 0 |
| BRAWH3004350 | 16.580 | 0 | 0 | 0 | 0 | 0 | 18.386 |
| BRAWH3004453 | 100 | 0 | 0 | 0 | 0 | 0 | 0 |
| BRAWH3004530 | 17.640 | 0 | 0 | 17.997 | 0 | 0 | 19.562 |
| BRAWH3004666 | 49.499 | 0 | 0 | 50.501 | 0 | 0 | 0 |
| BRAWH3005037 | 100 | 0 | 0 | 0 | 0 | 0 | 0 |
| BRAWH3005047 | 100 | 0 | 0 | 0 | 0 | 0 | 0 |
| BRAWH3005132 | 49.811 | 0 | 0 | 0 | 0 | 0 | 0 |
| BRAWH3005146 | 10.127 | 0 | 0 | 10.331 | 0 | 0 | 5.615 |
| BRAWH3005292 | 100 | 0 | 0 | 0 | 0 | 0 | 0 |
| BRAWH3005300 | 47.167 | 0 | 0 | 0 | 0 | 0 | 0 |
| BRAWH3005422 | 100 | 0 | 0 | 0 | 0 | 0 | 0 |
| BRAWH3005534 | 100 | 0 | 0 | 0 | 0 | 0 | 0 |
| BRAWH3005886 | 100 | 0 | 0 | 0 | 0 | 0 | 0 |
| BRAWH3005892 | 100 | 0 | 0 | 0 | 0 | 0 | 0 |
| BRAWH3005896 | 100 | 0 | 0 | 0 | 0 | 0 | 0 |
| BRAWH3005912 | 100 | 0 | 0 | 0 | 0 | 0 | 0 |
| BRAWH3005981 | 29.324 | 0 | 0 | 0 | 0 | 0 | 0 |
| BRAWH3006547 | 100 | 0 | 0 | 0 | 0 | 0 | 0 |
| BRAWH3006548 | 71.018 | 0 | 0 | 28.982 | 0 | 0 | 0 |
| BRAWH3006761 | 21.269 | 0 | 0 | 43.398 | 0 | 0 | 0 |
| BRAWH3006792 | 49.499 | 0 | 0 | 50.501 | 0 | 0 | 0 |
| BRAWH3007129 | 29.521 | 0 | 0 | 10.040 | 0 | 0 | 10.913 |
| BRAWH3007221 | 100 | 0 | 0 | 0 | 0 | 0 | 0 |
| BRAWH3007441 | 100 | 0 | 0 | 0 | 0 | 0 | 0 |
| BRAWH3007455 | 100 | 0 | 0 | 0 | 0 | 0 | 0 |
| BRAWH3007506 | 100 | 0 | 0 | 0 | 0 | 0 | 0 |
| BRAWH3007726 | 54.530 | 0 | 0 | 0 | 0 | 0 | 0 |
| BRAWH3007783 | 100 | 0 | 0 | 0 | 0 | 0 | 0 |
| BRAWH3008167 | 100 | 0 | 0 | 0 | 0 | 0 | 0 |
| BRAWH3008341 | 100 | 0 | 0 | 0 | 0 | 0 | 0 |
| BRAWH3008559 | 100 | 0 | 0 | 0 | 0 | 0 | 0 |
| BRAWH3008697 | 100 | 0 | 0 | 0 | 0 | 0 | 0 |
| BRAWH3008867 | 100 | 0 | 0 | 0 | 0 | 0 | 0 |
| BRAWH3008931 | 3.463 | 15.892 | 0 | 10.601 | 12.891 | 0 | 0 |
| BRAWH3009017 | 100 | 0 | 0 | 0 | 0 | 0 | 0 |
| BRAWH3009285 | 73.807 | 0 | 0 | 0 | 0 | 0 | 0 |
| BRAWH3009297 | 58.488 | 0 | 0 | 0 | 0 | 0 | 0 |
| BRAWH3009546 | 70.717 | 0 | 0 | 0 | 0 | 0 | 0 |
| BRAWH3009961 | 47.417 | 0 | 0 | 0 | 0 | 0 | 52.583 |
| BRAWH3010461 | 16.435 | 0 | 7.278 | 8.384 | 7.646 | 0 | 11.391 |
| BRAWH3010560 | 100 | 0 | 0 | 0 | 0 | 0 | 0 |
| BRAWH3010602 | 100 | 0 | 0 | 0 | 0 | 0 | 0 |
| BRAWH3010657 | 100 | 0 | 0 | 0 | 0 | 0 | 0 |
| BRAWH3010726 | 100 | 0 | 0 | 0 | 0 | 0 | 0 |
| BRAWH3010833 | 100 | 0 | 0 | 0 | 0 | 0 | 0 |
| BRAWH3011101 | 100 | 0 | 0 | 0 | 0 | 0 | 0 |
| BRAWH3011331 | 100 | 0 | 0 | 0 | 0 | 0 | 0 |
| BRAWH3011402 | 100 | 0 | 0 | 0 | 0 | 0 | 0 |
| BRAWH3011577 | 100 | 0 | 0 | 0 | 0 | 0 | 0 |
| BRAWH3011623 | 100 | 0 | 0 | 0 | 0 | 0 | 0 |
| BRAWH3011637 | 30.862 | 0 | 0 | 0 | 0 | 0 | 0 |
| BRAWH3011685 | 100 | 0 | 0 | 0 | 0 | 0 | 0 |
| BRAWH3011907 | 47.417 | 0 | 0 | 0 | 0 | 0 | 52.583 |
| BRAWH3011929 | 100 | 0 | 0 | 0 | 0 | 0 | 0 |
| BRAWH3012005 | 100 | 0 | 0 | 0 | 0 | 0 | 0 |
| BRAWH3012662 | 33.972 | 0 | 0 | 11.553 | 0 | 0 | 12.558 |
| BRAWH3012779 | 100 | 0 | 0 | 0 | 0 | 0 | 0 |
| BRAWH3013009 | 100 | 0 | 0 | 0 | 0 | 0 | 0 |
| BRAWH3013049 | 100 | 0 | 0 | 0 | 0 | 0 | 0 |
| BRAWH3013197 | 100 | 0 | 0 | 0 | 0 | 0 | 0 |
| BRAWH3013264 | 100 | 0 | 0 | 0 | 0 | 0 | 0 |
| BRAWH3013508 | 100 | 0 | 0 | 0 | 0 | 0 | 0 |
| BRAWH3014609 | 44.597 | 0 | 0 | 0 | 0 | 0 | 0 |
| BRAWH3014639 | 100 | 0 | 0 | 0 | 0 | 0 | 0 |
| BRAWH3015017 | 100 | 0 | 0 | 0 | 0 | 0 | 0 |
| BRAWH3015175 | 100 | 0 | 0 | 0 | 0 | 0 | 0 |
| BRAWH3015260 | 100 | 0 | 0 | 0 | 0 | 0 | 0 |
| BRAWH3015610 | 100 | 0 | 0 | 0 | 0 | 0 | 0 |
| BRAWH3015825 | 100 | 0 | 0 | 0 | 0 | 0 | 0 |
| BRAWH3016123 | 100 | 0 | 0 | 0 | 0 | 0 | 0 |
| BRAWH3016271 | 5.394 | 12.374 | 0 | 0 | 0 | 0 | 0 |
| BRAWH3016715 | 100 | 0 | 0 | 0 | 0 | 0 | 0 |
| BRAWH3017259 | 13.996 | 0 | 0 | 0 | 0 | 0 | 0 |
| BRAWH3017260 | 100 | 0 | 0 | 0 | 0 | 0 | 0 |
| BRAWH3017477 | 100 | 0 | 0 | 0 | 0 | 0 | 0 |
| BRAWH3017537 | 100 | 0 | 0 | 0 | 0 | 0 | 0 |
| BRAWH3017980 | 49.811 | 0 | 0 | 0 | 0 | 0 | 0 |
| BRAWH3018063 | 15.836 | 0 | 0 | 0 | 0 | 0 | 0 |
| BRAWH3018369 | 100 | 0 | 0 | 0 | 0 | 0 | 0 |
| BRAWH3018548 | 100 | 0 | 0 | 0 | 0 | 0 | 0 |
| BRAWH3018969 | 100 | 0 | 0 | 0 | 0 | 0 | 0 |
| BRAWH3019026 | 100 | 0 | 0 | 0 | 0 | 0 | 0 |
| BRAWH3019529 | 100 | 0 | 0 | 0 | 0 | 0 | 0 |
| BRAWH3019594 | 100 | 0 | 0 | 0 | 0 | 0 | 0 |
| BRAWH3019629 | 100 | 0 | 0 | 0 | 0 | 0 | 0 |
| BRAWH3019820 | 100 | 0 | 0 | 0 | 0 | 0 | 0 |
| BRAWH3020200 | 100 | 0 | 0 | 0 | 0 | 0 | 0 |
| BRAWH3020318 | 100 | 0 | 0 | 0 | 0 | 0 | 0 |
| BRAWH3020486 | 100 | 0 | 0 | 0 | 0 | 0 | 0 |
| BRAWH3020884 | 100 | 0 | 0 | 0 | 0 | 0 | 0 |
| BRAWH3020928 | 49.499 | 0 | 0 | 50.501 | 0 | 0 | 0 |
| BRAWH3021012 | 100 | 0 | 0 | 0 | 0 | 0 | 0 |
| BRAWH3021545 | 7.296 | 0 | 0 | 3.722 | 0 | 0 | 0 |
| BRAWH3021574 | 9.400 | 0 | 0 | 0 | 17.492 | 0 | 0 |
| BRAWH3021580 | 100 | 0 | 0 | 0 | 0 | 0 | 0 |
| BRAWH3021641 | 100 | 0 | 0 | 0 | 0 | 0 | 0 |
| BRAWH3021643 | 100 | 0 | 0 | 0 | 0 | 0 | 0 |
| BRAWH3021724 | 100 | 0 | 0 | 0 | 0 | 0 | 0 |
| BRAWH3022347 | 100 | 0 | 0 | 0 | 0 | 0 | 0 |
| BRAWH3022431 | 100 | 0 | 0 | 0 | 0 | 0 | 0 |
| BRAWH3022459 | 100 | 0 | 0 | 0 | 0 | 0 | 0 |
| BRAWH3022542 | 100 | 0 | 0 | 0 | 0 | 0 | 0 |
| BRAWH3022719 | 100 | 0 | 0 | 0 | 0 | 0 | 0 |
| BRAWH3022866 | 100 | 0 | 0 | 0 | 0 | 0 | 0 |
| BRAWH3022900 | 100 | 0 | 0 | 0 | 0 | 0 | 0 |
| BRAWH3023156 | 100 | 0 | 0 | 0 | 0 | 0 | 0 |
| BRAWH3023168 | 100 | 0 | 0 | 0 | 0 | 0 | 0 |
| BRAWH3023172 | 0.916 | 2.101 | 0 | 1.869 | 0 | 3.302 | 2.031 |
| BRAWH3023274 | 100 | 0 | 0 | 0 | 0 | 0 | 0 |
| BRAWH3023421 | 100 | 0 | 0 | 0 | 0 | 0 | 0 |
| BRAWH3024231 | 100 | 0 | 0 | 0 | 0 | 0 | 0 |
| BRAWH3024242 | 31.843 | 0 | 0 | 32.488 | 0 | 0 | 0 |
| BRAWH3024469 | 100 | 0 | 0 | 0 | 0 | 0 | 0 |
| BRAWH3024506 | 100 | 0 | 0 | 0 | 0 | 0 | 0 |
| BRAWH3024613 | 100 | 0 | 0 | 0 | 0 | 0 | 0 |
| BRAWH3024989 | 2.369 | 0 | 0 | 4.834 | 0 | 0 | 0 |
| BRAWH3026349 | 100 | 0 | 0 | 0 | 0 | 0 | 0 |
| BRAWH3026394 | 100 | 0 | 0 | 0 | 0 | 0 | 0 |
| BRAWH3026529 | 45.606 | 0 | 0 | 0 | 0 | 0 | 0 |
| BRAWH3027420 | 100 | 0 | 0 | 0 | 0 | 0 | 0 |
| BRAWH3027440 | 58.488 | 0 | 0 | 0 | 0 | 0 | 0 |
| BRAWH3027533 | 11.008 | 0 | 0 | 0 | 0 | 0 | 0 |
| BRAWH3027574 | 47.417 | 0 | 0 | 0 | 0 | 0 | 52.583 |
| BRAWH3027607 | 100 | 0 | 0 | 0 | 0 | 0 | 0 |
| BRAWH3027616 | 100 | 0 | 0 | 0 | 0 | 0 | 0 |
| BRAWH3027675 | 100 | 0 | 0 | 0 | 0 | 0 | 0 |
| BRAWH3027806 | 100 | 0 | 0 | 0 | 0 | 0 | 0 |
| BRAWH3027880 | 100 | 0 | 0 | 0 | 0 | 0 | 0 |
| BRAWH3027927 | 100 | 0 | 0 | 0 | 0 | 0 | 0 |
| BRAWH3028202 | 100 | 0 | 0 | 0 | 0 | 0 | 0 |
| BRAWH3028223 | 100 | 0 | 0 | 0 | 0 | 0 | 0 |
| BRAWH3028306 | 100 | 0 | 0 | 0 | 0 | 0 | 0 |
| BRAWH3028461 | 100 | 0 | 0 | 0 | 0 | 0 | 0 |
| BRAWH3028645 | 100 | 0 | 0 | 0 | 0 | 0 | 0 |
| BRAWH3028754 | 100 | 0 | 0 | 0 | 0 | 0 | 0 |
| BRAWH3028796 | 100 | 0 | 0 | 0 | 0 | 0 | 0 |
| BRAWH3029254 | 100 | 0 | 0 | 0 | 0 | 0 | 0 |
| BRAWH3029313 | 100 | 0 | 0 | 0 | 0 | 0 | 0 |
| BRAWH3029538 | 100 | 0 | 0 | 0 | 0 | 0 | 0 |
| BRAWH3029556 | 38.935 | 0 | 0 | 19.861 | 0 | 0 | 21.589 |
| BRAWH3029806 | 26.735 | 0 | 0 | 0 | 0 | 0 | 9.883 |
| BRAWH3030613 | 100 | 0 | 0 | 0 | 0 | 0 | 0 |
| BRAWH3030772 | 100 | 0 | 0 | 0 | 0 | 0 | 0 |
| BRAWH3030810 | 21.287 | 0 | 0 | 0 | 0 | 0 | 0 |
| BRAWH3030892 | 100 | 0 | 0 | 0 | 0 | 0 | 0 |
| BRAWH3030910 | 100 | 0 | 0 | 0 | 0 | 0 | 0 |
| BRAWH3030950 | 100 | 0 | 0 | 0 | 0 | 0 | 0 |
| BRAWH3031054 | 100 | 0 | 0 | 0 | 0 | 0 | 0 |
| BRAWH3031710 | 100 | 0 | 0 | 0 | 0 | 0 | 0 |
| BRAWH3031902 | 100 | 0 | 0 | 0 | 0 | 0 | 0 |
| BRAWH3032298 | 100 | 0 | 0 | 0 | 0 | 0 | 0 |
| BRAWH3032340 | 100 | 0 | 0 | 0 | 0 | 0 | 0 |
| BRAWH3032571 | 100 | 0 | 0 | 0 | 0 | 0 | 0 |
| BRAWH3032620 | 100 | 0 | 0 | 0 | 0 | 0 | 0 |
| BRAWH3032930 | 100 | 0 | 0 | 0 | 0 | 0 | 0 |
| BRAWH3033117 | 100 | 0 | 0 | 0 | 0 | 0 | 0 |
| BRAWH3033293 | 100 | 0 | 0 | 0 | 0 | 0 | 0 |
| BRAWH3033448 | 100 | 0 | 0 | 0 | 0 | 0 | 0 |
| BRAWH3033513 | 100 | 0 | 0 | 0 | 0 | 0 | 0 |
| BRAWH3034097 | 100 | 0 | 0 | 0 | 0 | 0 | 0 |
| BRAWH3034114 | 100 | 0 | 0 | 0 | 0 | 0 | 0 |
| BRAWH3034134 | 100 | 0 | 0 | 0 | 0 | 0 | 0 |
| BRAWH3034668 | 100 | 0 | 0 | 0 | 0 | 0 | 0 |
| BRAWH3034743 | 16.662 | 0 | 14.757 | 21.249 | 0 | 0 | 18.478 |
| BRAWH3034775 | 100 | 0 | 0 | 0 | 0 | 0 | 0 |
| BRAWH3034890 | 100 | 0 | 0 | 0 | 0 | 0 | 0 |
| BRAWH3035154 | 100 | 0 | 0 | 0 | 0 | 0 | 0 |
| BRAWH3035403 | 35.337 | 0 | 0 | 0 | 0 | 0 | 0 |
| BRAWH3035904 | 100 | 0 | 0 | 0 | 0 | 0 | 0 |
| BRAWH3035914 | 100 | 0 | 0 | 0 | 0 | 0 | 0 |
| BRAWH3035936 | 70.575 | 0 | 0 | 0 | 0 | 0 | 0 |
| BRAWH3036077 | 100 | 0 | 0 | 0 | 0 | 0 | 0 |
| BRAWH3036247 | 54.530 | 0 | 0 | 0 | 0 | 0 | 0 |
| BRAWH3036270 | 100 | 0 | 0 | 0 | 0 | 0 | 0 |
| BRAWH3036561 | 100 | 0 | 0 | 0 | 0 | 0 | 0 |
| BRAWH3036738 | 100 | 0 | 0 | 0 | 0 | 0 | 0 |
| BRAWH3036951 | 100 | 0 | 0 | 0 | 0 | 0 | 0 |
| BRAWH3037265 | 44.597 | 0 | 0 | 0 | 0 | 0 | 0 |
| BRAWH3037394 | 100 | 0 | 0 | 0 | 0 | 0 | 0 |
| BRAWH3037533 | 100 | 0 | 0 | 0 | 0 | 0 | 0 |
| BRAWH3037979 | 100 | 0 | 0 | 0 | 0 | 0 | 0 |
| BRAWH3038055 | 100 | 0 | 0 | 0 | 0 | 0 | 0 |
| BRAWH3038230 | 100 | 0 | 0 | 0 | 0 | 0 | 0 |
| BRAWH3038252 | 100 | 0 | 0 | 0 | 0 | 0 | 0 |
| BRAWH3038324 | 100 | 0 | 0 | 0 | 0 | 0 | 0 |
| BRCAN1000065 | 0 | 100 | 0 | 0 | 0 | 0 | 0 |
| BRCAN1000076 | 0 | 6.753 | 0 | 0 | 0 | 0 | 0 |
| BRCAN1000105 | 0 | 100 | 0 | 0 | 0 | 0 | 0 |
| BRCAN1000149 | 0 | 56.874 | 0 | 0 | 0 | 0 | 0 |
| BRCAN1000168 | 5.643 | 9.709 | 0 | 17.271 | 5.250 | 10.173 | 7.822 |
| BRCAN2000148 | 0 | 100 | 0 | 0 | 0 | 0 | 0 |
| BRCAN2000149 | 0 | 100 | 0 | 0 | 0 | 0 | 0 |
| BRCAN2000209 | 0 | 38.263 | 0 | 0 | 0 | 0 | 0 |
| BRCAN2000239 | 0 | 100 | 0 | 0 | 0 | 0 | 0 |
| BRCAN2000346 | 0 | 100 | 0 | 0 | 0 | 0 | 0 |
| BRCAN2000364 | 0 | 100 | 0 | 0 | 0 | 0 | 0 |
| BRCAN2000383 | 0 | 100 | 0 | 0 | 0 | 0 | 0 |
| BRCAN2000418 | 0 | 100 | 0 | 0 | 0 | 0 | 0 |
| BRCAN2000523 | 0 | 100 | 0 | 0 | 0 | 0 | 0 |
| BRCAN2000541 | 0 | 100 | 0 | 0 | 0 | 0 | 0 |
| BRCAN2000563 | 13.150 | 30.170 | 0 | 0 | 0 | 0 | 0 |
| BRCAN2000577 | 0 | 100 | 0 | 0 | 0 | 0 | 0 |
| BRCAN2000620 | 0 | 100 | 0 | 0 | 0 | 0 | 0 |
| BRCAN2000639 | 0 | 38.481 | 0 | 0 | 0 | 0 | 0 |
| BRCAN2000763 | 0 | 100 | 0 | 0 | 0 | 0 | 0 |
| BRCAN2000832 | 0 | 100 | 0 | 0 | 0 | 0 | 0 |
| BRCAN2000923 | 0 | 2.660 | 0 | 0 | 0 | 0 | 0 |
| BRCAN2001223 | 0 | 53.082 | 0 | 23.605 | 0 | 0 | 0 |
| BRCAN2001866 | 0 | 100 | 0 | 0 | 0 | 0 | 0 |
| BRCAN2002173 | 0 | 9.124 | 7.044 | 0 | 7.401 | 0 | 0 |
| BRCAN2002416 | 0 | 100 | 0 | 0 | 0 | 0 | 0 |
| BRCAN2002473 | 0 | 100 | 0 | 0 | 0 | 0 | 0 |
| BRCAN2002562 | 0 | 100 | 0 | 0 | 0 | 0 | 0 |
| BRCAN2002662 | 0 | 100 | 0 | 0 | 0 | 0 | 0 |
| BRCAN2002826 | 0 | 100 | 0 | 0 | 0 | 0 | 0 |
| BRCAN2002854 | 0 | 100 | 0 | 0 | 0 | 0 | 0 |
| BRCAN2002856 | 0 | 39.306 | 60.694 | 0 | 0 | 0 | 0 |
| BRCAN2002892 | 0 | 67.414 | 0 | 0 | 0 | 0 | 32.586 |
| BRCAN2002944 | 0 | 73.343 | 0 | 0 | 0 | 0 | 0 |
| BRCAN2002948 | 0 | 100 | 0 | 0 | 0 | 0 | 0 |
| BRCAN2003070 | 0 | 100 | 0 | 0 | 0 | 0 | 0 |
| BRCAN2003269 | 0 | 100 | 0 | 0 | 0 | 0 | 0 |
| BRCAN2003389 | 0 | 6.976 | 0 | 0 | 0 | 0 | 0 |
| BRCAN2003628 | 0 | 100 | 0 | 0 | 0 | 0 | 0 |
| BRCAN2003703 | 0 | 100 | 0 | 0 | 0 | 0 | 0 |
| BRCAN2003746 | 0 | 100 | 0 | 0 | 0 | 0 | 0 |
| BRCAN2003814 | 0 | 9.924 | 0 | 0 | 0 | 0 | 0 |
| BRCAN2003944 | 0.282 | 3.230 | 0.998 | 0 | 1.048 | 2.031 | 0.625 |
| BRCAN2003987 | 0 | 100 | 0 | 0 | 0 | 0 | 0 |
| BRCAN2004173 | 0 | 100 | 0 | 0 | 0 | 0 | 0 |
| BRCAN2004355 | 0 | 100 | 0 | 0 | 0 | 0 | 0 |
| BRCAN2004371 | 0 | 100 | 0 | 0 | 0 | 0 | 0 |
| BRCAN2004653 | 0 | 77.826 | 0 | 0 | 0 | 0 | 0 |
| BRCAN2004699 | 0 | 100 | 0 | 0 | 0 | 0 | 0 |
| BRCAN2005436 | 0 | 100 | 0 | 0 | 0 | 0 | 0 |
| BRCAN2005449 | 0 | 100 | 0 | 0 | 0 | 0 | 0 |
| BRCAN2006019 | 0 | 25.857 | 26.617 | 3.833 | 0 | 13.546 | 0 |
| BRCAN2006051 | 0 | 100 | 0 | 0 | 0 | 0 | 0 |
| BRCAN2006063 | 0 | 100 | 0 | 0 | 0 | 0 | 0 |
| BRCAN2006117 | 0 | 57.190 | 0 | 0 | 0 | 0 | 0 |
| BRCAN2006174 | 0 | 100 | 0 | 0 | 0 | 0 | 0 |
| BRCAN2006290 | 0 | 100 | 0 | 0 | 0 | 0 | 0 |
| BRCAN2006297 | 0 | 33.031 | 51.004 | 0 | 0 | 0 | 15.966 |
| BRCAN2006323 | 0 | 100 | 0 | 0 | 0 | 0 | 0 |
| BRCAN2006401 | 0 | 52.014 | 0 | 0 | 0 | 0 | 25.142 |
| BRCAN2006411 | 0 | 100 | 0 | 0 | 0 | 0 | 0 |
| BRCAN2006450 | 0 | 100 | 0 | 0 | 0 | 0 | 0 |
| BRCAN2006955 | 0 | 100 | 0 | 0 | 0 | 0 | 0 |
| BRCAN2007119 | 0 | 38.885 | 0 | 0 | 0 | 61.115 | 0 |
| BRCAN2007144 | 0 | 100 | 0 | 0 | 0 | 0 | 0 |
| BRCAN2007409 | 0 | 6.883 | 10.629 | 0 | 0 | 0 | 0 |
| BRCAN2007426 | 0 | 100 | 0 | 0 | 0 | 0 | 0 |
| BRCAN2007525 | 0 | 100 | 0 | 0 | 0 | 0 | 0 |
| BRCAN2007700 | 0 | 27.900 | 0 | 0 | 0 | 0 | 0 |
| BRCAN2008494 | 0 | 100 | 0 | 0 | 0 | 0 | 0 |
| BRCAN2008528 | 0 | 100 | 0 | 0 | 0 | 0 | 0 |
| BRCAN2008701 | 0 | 100 | 0 | 0 | 0 | 0 | 0 |
| BRCAN2008894 | 0 | 17.467 | 0 | 0 | 0 | 0 | 0 |
| BRCAN2009156 | 0 | 100 | 0 | 0 | 0 | 0 | 0 |
| BRCAN2009168 | 0 | 100 | 0 | 0 | 0 | 0 | 0 |
| BRCAN2009203 | 0 | 100 | 0 | 0 | 0 | 0 | 0 |
| BRCAN2009432 | 0 | 3.021 | 0 | 0 | 0 | 0 | 0 |
| BRCAN2010374 | 0 | 69.484 | 0 | 0 | 0 | 0 | 0 |
| BRCAN2010376 | 0 | 76.373 | 0 | 0 | 0 | 0 | 0 |
| BRCAN2010547 | 0 | 100 | 0 | 0 | 0 | 0 | 0 |
| BRCAN2010581 | 0 | 100 | 0 | 0 | 0 | 0 | 0 |
| BRCAN2010665 | 19.610 | 29.993 | 0 | 13.338 | 0 | 11.785 | 7.249 |
| BRCAN2011254 | 0 | 100 | 0 | 0 | 0 | 0 | 0 |
| BRCAN2011316 | 0 | 100 | 0 | 0 | 0 | 0 | 0 |
| BRCAN2011602 | 0 | 100 | 0 | 0 | 0 | 0 | 0 |
| BRCAN2011946 | 0 | 14.508 | 0 | 0 | 23.536 | 0 | 0 |
| BRCAN2012110 | 0 | 100 | 0 | 0 | 0 | 0 | 0 |
| BRCAN2012355 | 0 | 100 | 0 | 0 | 0 | 0 | 0 |
| BRCAN2012402 | 0 | 100 | 0 | 0 | 0 | 0 | 0 |
| BRCAN2012408 | 0 | 100 | 0 | 0 | 0 | 0 | 0 |
| BRCAN2012481 | 0 | 100 | 0 | 0 | 0 | 0 | 0 |
| BRCAN2012613 | 0 | 100 | 0 | 0 | 0 | 0 | 0 |
| BRCAN2012713 | 0 | 100 | 0 | 0 | 0 | 0 | 0 |
| BRCAN2012929 | 0 | 55.996 | 0 | 0 | 0 | 44.004 | 0 |
| BRCAN2013655 | 0 | 100 | 0 | 0 | 0 | 0 | 0 |
| BRCAN2013660 | 0 | 100 | 0 | 0 | 0 | 0 | 0 |
| BRCAN2014143 | 0 | 100 | 0 | 0 | 0 | 0 | 0 |
| BRCAN2014229 | 0 | 100 | 0 | 0 | 0 | 0 | 0 |
| BRCAN2014370 | 0 | 100 | 0 | 0 | 0 | 0 | 0 |
| BRCAN2014602 | 0 | 100 | 0 | 0 | 0 | 0 | 0 |
| BRCAN2014652 | 0 | 100 | 0 | 0 | 0 | 0 | 0 |
| BRCAN2014788 | 0 | 100 | 0 | 0 | 0 | 0 | 0 |
| BRCAN2014881 | 0 | 69.484 | 0 | 0 | 0 | 0 | 0 |
| BRCAN2014952 | 0 | 100 | 0 | 0 | 0 | 0 | 0 |
| BRCAN2015371 | 0 | 25.149 | 0 | 0 | 0 | 0 | 0 |
| BRCAN2015402 | 0 | 100 | 0 | 0 | 0 | 0 | 0 |
| BRCAN2015464 | 0 | 100 | 0 | 0 | 0 | 0 | 0 |
| BRCAN2015719 | 3.019 | 6.927 | 0 | 0 | 0 | 0 | 3.348 |
| BRCAN2015757 | 0 | 100 | 0 | 0 | 0 | 0 | 0 |
| BRCAN2015886 | 0 | 100 | 0 | 0 | 0 | 0 | 0 |
| BRCAN2016025 | 0 | 100 | 0 | 0 | 0 | 0 | 0 |
| BRCAN2016046 | 0 | 69.484 | 0 | 0 | 0 | 0 | 0 |
| BRCAN2016174 | 0 | 24.944 | 0 | 0 | 0 | 0 | 12.057 |
| BRCAN2016433 | 0 | 100 | 0 | 0 | 0 | 0 | 0 |
| BRCAN2016619 | 0 | 30.224 | 0 | 0 | 0 | 0 | 0 |
| BRCAN2016762 | 0 | 100 | 0 | 0 | 0 | 0 | 0 |
| BRCAN2016814 | 0 | 67.414 | 0 | 0 | 0 | 0 | 32.586 |
| BRCAN2017442 | 0 | 100 | 0 | 0 | 0 | 0 | 0 |
| BRCAN2017905 | 0 | 100 | 0 | 0 | 0 | 0 | 0 |
| BRCAN2018240 | 0 | 100 | 0 | 0 | 0 | 0 | 0 |
| BRCAN2018269 | 0 | 100 | 0 | 0 | 0 | 0 | 0 |
| BRCAN2018490 | 0 | 100 | 0 | 0 | 0 | 0 | 0 |
| BRCAN2018566 | 0 | 56.874 | 0 | 0 | 0 | 0 | 0 |
| BRCAN2018667 | 0 | 100 | 0 | 0 | 0 | 0 | 0 |
| BRCAN2018748 | 0 | 100 | 0 | 0 | 0 | 0 | 0 |
| BRCAN2018935 | 0 | 20.928 | 0 | 0 | 0 | 0 | 0 |
| BRCAN2019002 | 0 | 100 | 0 | 0 | 0 | 0 | 0 |
| BRCAN2019387 | 0 | 100 | 0 | 0 | 0 | 0 | 0 |
| BRCAN2019653 | 0 | 57.233 | 0 | 0 | 0 | 0 | 0 |
| BRCAN2019773 | 0 | 100 | 0 | 0 | 0 | 0 | 0 |
| BRCAN2019907 | 0 | 100 | 0 | 0 | 0 | 0 | 0 |
| BRCAN2020234 | 0 | 56.552 | 0 | 0 | 0 | 0 | 27.335 |
| BRCAN2020331 | 0 | 100 | 0 | 0 | 0 | 0 | 0 |
| BRCAN2020412 | 0 | 25.021 | 0 | 0 | 0 | 0 | 0 |
| BRCAN2020467 | 0 | 100 | 0 | 0 | 0 | 0 | 0 |
| BRCAN2020710 | 0 | 49.868 | 0 | 22.176 | 0 | 0 | 0 |
| BRCAN2020880 | 0 | 100 | 0 | 0 | 0 | 0 | 0 |
| BRCAN2021024 | 10.255 | 23.527 | 0 | 10.462 | 0 | 0 | 22.744 |
| BRCAN2021028 | 0 | 24.138 | 37.272 | 0 | 0 | 0 | 0 |
| BRCAN2021325 | 0 | 100 | 0 | 0 | 0 | 0 | 0 |
| BRCAN2021452 | 0 | 100 | 0 | 0 | 0 | 0 | 0 |
| BRCAN2021669 | 0 | 100 | 0 | 0 | 0 | 0 | 0 |
| BRCAN2021718 | 0 | 100 | 0 | 0 | 0 | 0 | 0 |
| BRCAN2022126 | 4.855 | 22.279 | 17.201 | 4.954 | 0 | 0 | 0 |
| BRCAN2022191 | 0 | 100 | 0 | 0 | 0 | 0 | 0 |
| BRCAN2022472 | 0 | 100 | 0 | 0 | 0 | 0 | 0 |
| BRCAN2022474 | 0 | 100 | 0 | 0 | 0 | 0 | 0 |
| BRCAN2023347 | 0 | 100 | 0 | 0 | 0 | 0 | 0 |
| BRCAN2023724 | 0 | 100 | 0 | 0 | 0 | 0 | 0 |
| BRCAN2024451 | 0 | 100 | 0 | 0 | 0 | 0 | 0 |
| BRCAN2024563 | 0 | 100 | 0 | 0 | 0 | 0 | 0 |
| BRCAN2024572 | 0 | 6.266 | 0 | 2.786 | 10.165 | 0 | 6.057 |
| BRCAN2025009 | 0 | 100 | 0 | 0 | 0 | 0 | 0 |
| BRCAN2025093 | 21.732 | 49.857 | 0 | 0 | 0 | 0 | 0 |
| BRCAN2025349 | 0 | 100 | 0 | 0 | 0 | 0 | 0 |
| BRCAN2025368 | 0 | 38.835 | 0 | 0 | 0 | 0 | 0 |
| BRCAN2025712 | 0 | 100 | 0 | 0 | 0 | 0 | 0 |
| BRCAN2025982 | 0 | 69.219 | 0 | 30.781 | 0 | 0 | 0 |
| BRCAN2026117 | 0 | 100 | 0 | 0 | 0 | 0 | 0 |
| BRCAN2026197 | 6.245 | 3.582 | 0 | 6.371 | 0 | 5.629 | 8.657 |
| BRCAN2026340 | 0 | 34.670 | 0 | 0 | 0 | 0 | 0 |
| BRCAN2026711 | 0 | 100 | 0 | 0 | 0 | 0 | 0 |
| BRCAN2027150 | 0 | 23.385 | 0 | 0 | 0 | 0 | 0 |
| BRCAN2027310 | 0 | 100 | 0 | 0 | 0 | 0 | 0 |
| BRCAN2027334 | 0 | 69.484 | 0 | 0 | 0 | 0 | 0 |
| BRCAN2027355 | 0 | 100 | 0 | 0 | 0 | 0 | 0 |
| BRCAN2027364 | 2.970 | 20.440 | 21.042 | 0 | 11.054 | 0 | 13.174 |
| BRCAN2027513 | 0 | 100 | 0 | 0 | 0 | 0 | 0 |
| BRCAN2027593 | 0 | 100 | 0 | 0 | 0 | 0 | 0 |
| BRCAN2027970 | 0 | 100 | 0 | 0 | 0 | 0 | 0 |
| BRCAN2028021 | 0 | 100 | 0 | 0 | 0 | 0 | 0 |
| BRCAN2028036 | 0 | 5.539 | 0 | 2.463 | 0 | 0 | 0 |
| BRCAN2028040 | 0 | 60.666 | 0 | 0 | 0 | 0 | 0 |
| BRCAN2028319 | 0 | 29.858 | 0 | 0 | 48.439 | 0 | 0 |
| BRCAN2028338 | 0 | 100 | 0 | 0 | 0 | 0 | 0 |
| BRCAN2028355 | 0 | 3.389 | 0 | 1.507 | 0 | 0 | 0 |
| BRCAN2028378 | 0 | 100 | 0 | 0 | 0 | 0 | 0 |
| BRCAN2028460 | 0 | 100 | 0 | 0 | 0 | 0 | 0 |
| BRCAN2028545 | 0 | 38.885 | 0 | 0 | 0 | 61.115 | 0 |
| BRCAN2028634 | 0 | 67.414 | 0 | 0 | 0 | 0 | 32.586 |
| BRCAN2028702 | 0 | 100 | 0 | 0 | 0 | 0 | 0 |
| BRCOC1000040 | 0 | 0 | 10.447 | 9.026 | 0 | 0 | 6.541 |
| BRCOC1000087 | 0 | 0 | 100 | 0 | 0 | 0 | 0 |
| BRCOC1000094 | 0.952 | 8.738 | 16.865 | 4.857 | 21.263 | 3.433 | 5.280 |
| BRCOC2000004 | 1.800 | 20.649 | 12.754 | 5.509 | 20.099 | 12.981 | 5.989 |
| BRCOC2000047 | 0 | 0 | 49.505 | 0 | 0 | 0 | 0 |
| BRCOC2000152 | 0 | 0 | 74.037 | 0 | 0 | 0 | 0 |
| BRCOC2000184 | 0 | 0 | 29.688 | 8.550 | 0 | 0 | 0 |
| BRCOC2000186 | 0 | 0 | 10.537 | 0 | 0 | 0 | 0 |
| BRCOC2000333 | 0 | 0 | 42.433 | 0 | 0 | 0 | 0 |
| BRCOC2000360 | 17.976 | 0 | 63.683 | 18.340 | 0 | 0 | 0 |
| BRCOC2000404 | 0 | 0 | 100 | 0 | 0 | 0 | 0 |
| BRCOC2000487 | 0 | 0.665 | 1.026 | 0 | 0 | 0 | 0.643 |
| BRCOC2000637 | 0 | 0 | 100 | 0 | 0 | 0 | 0 |
| BRCOC2000670 | 0 | 0 | 77.856 | 0 | 0 | 0 | 0 |
| BRCOC2000816 | 22.014 | 0 | 77.986 | 0 | 0 | 0 | 0 |
| BRCOC2000850 | 0 | 0 | 100 | 0 | 0 | 0 | 0 |
| BRCOC2001355 | 0 | 0 | 100 | 0 | 0 | 0 | 0 |
| BRCOC2002085 | 0 | 0 | 100 | 0 | 0 | 0 | 0 |
| BRCOC2002155 | 0 | 0 | 75.977 | 0 | 0 | 0 | 0 |
| BRCOC2002323 | 0 | 0 | 100 | 0 | 0 | 0 | 0 |
| BRCOC2002659 | 0 | 0 | 100 | 0 | 0 | 0 | 0 |
| BRCOC2002664 | 0 | 0 | 100 | 0 | 0 | 0 | 0 |
| BRCOC2002686 | 0 | 0 | 100 | 0 | 0 | 0 | 0 |
| BRCOC2002751 | 0 | 0 | 100 | 0 | 0 | 0 | 0 |
| BRCOC2002756 | 0 | 0 | 100 | 0 | 0 | 0 | 0 |
| BRCOC2002777 | 0 | 0 | 100 | 0 | 0 | 0 | 0 |
| BRCOC2002835 | 0 | 0 | 100 | 0 | 0 | 0 | 0 |
| BRCOC2003100 | 0 | 0 | 83.309 | 0 | 0 | 0 | 0 |
| BRCOC2003125 | 0 | 0 | 20.131 | 0 | 0 | 0 | 0 |
| BRCOC2003187 | 0.786 | 1.804 | 2.786 | 0 | 5.854 | 2.836 | 0 |
| BRCOC2003213 | 10.381 | 0 | 36.777 | 0 | 0 | 0 | 0 |
| BRCOC2003513 | 0 | 0 | 100 | 0 | 0 | 0 | 0 |
| BRCOC2003732 | 0 | 0 | 75.977 | 0 | 0 | 0 | 0 |
| BRCOC2003740 | 0 | 0 | 100 | 0 | 0 | 0 | 0 |
| BRCOC2004036 | 0 | 0 | 49.505 | 0 | 0 | 0 | 0 |
| BRCOC2004175 | 0 | 0 | 34.465 | 0 | 0 | 0 | 0 |
| BRCOC2004354 | 0 | 0 | 100 | 0 | 0 | 0 | 0 |
| BRCOC2004455 | 0 | 0 | 100 | 0 | 0 | 0 | 0 |
| BRCOC2004950 | 0 | 0 | 100 | 0 | 0 | 0 | 0 |
| BRCOC2005542 | 0 | 0 | 62.599 | 0 | 0 | 0 | 19.596 |
| BRCOC2005903 | 0 | 0 | 31.097 | 0 | 0 | 0 | 0 |
| BRCOC2005951 | 0 | 0 | 100 | 0 | 0 | 0 | 0 |
| BRCOC2006164 | 0 | 0 | 51.486 | 0 | 0 | 0 | 32.234 |
| BRCOC2006189 | 6.639 | 15.231 | 23.518 | 3.387 | 0 | 11.969 | 18.405 |
| BRCOC2006243 | 0 | 0 | 100 | 0 | 0 | 0 | 0 |
| BRCOC2006270 | 0 | 0 | 49.505 | 0 | 0 | 0 | 0 |
| BRCOC2006639 | 0 | 0 | 100 | 0 | 0 | 0 | 0 |
| BRCOC2006942 | 0 | 0 | 100 | 0 | 0 | 0 | 0 |
| BRCOC2007034 | 0 | 0 | 83.309 | 0 | 0 | 0 | 0 |
| BRCOC2007593 | 0 | 0 | 100 | 0 | 0 | 0 | 0 |
| BRCOC2007733 | 0 | 0 | 100 | 0 | 0 | 0 | 0 |
| BRCOC2007769 | 0 | 0 | 100 | 0 | 0 | 0 | 0 |
| BRCOC2008064 | 0 | 0 | 100 | 0 | 0 | 0 | 0 |
| BRCOC2008225 | 0 | 0 | 100 | 0 | 0 | 0 | 0 |
| BRCOC2008642 | 0 | 0 | 100 | 0 | 0 | 0 | 0 |
| BRCOC2008993 | 0 | 0 | 100 | 0 | 0 | 0 | 0 |
| BRCOC2009052 | 0 | 0 | 100 | 0 | 0 | 0 | 0 |
| BRCOC2009196 | 0 | 0 | 100 | 0 | 0 | 0 | 0 |
| BRCOC2009380 | 0 | 0 | 100 | 0 | 0 | 0 | 0 |
| BRCOC2009638 | 0 | 0 | 100 | 0 | 0 | 0 | 0 |
| BRCOC2009937 | 0 | 0 | 56.917 | 16.392 | 0 | 0 | 0 |
| BRCOC2010115 | 0 | 0 | 100 | 0 | 0 | 0 | 0 |
| BRCOC2010123 | 0 | 2.513 | 3.881 | 0 | 0 | 0 | 0 |
| BRCOC2010510 | 0 | 0 | 85.082 | 0 | 0 | 0 | 0 |
| BRCOC2010730 | 0 | 0 | 38.292 | 0 | 0 | 0 | 11.987 |
| BRCOC2010980 | 0 | 0 | 100 | 0 | 0 | 0 | 0 |
| BRCOC2010994 | 0 | 0 | 100 | 0 | 0 | 0 | 0 |
| BRCOC2011010 | 0 | 0 | 100 | 0 | 0 | 0 | 0 |
| BRCOC2011398 | 0 | 0 | 100 | 0 | 0 | 0 | 0 |
| BRCOC2011418 | 0 | 0 | 100 | 0 | 0 | 0 | 0 |
| BRCOC2011506 | 3.806 | 2.183 | 3.371 | 1.942 | 0 | 0 | 4.221 |
| BRCOC2011769 | 0 | 0 | 100 | 0 | 0 | 0 | 0 |
| BRCOC2011996 | 0 | 0 | 100 | 0 | 0 | 0 | 0 |
| BRCOC2012172 | 0 | 0 | 9.965 | 2.870 | 0 | 0 | 3.119 |
| BRCOC2012229 | 0 | 0 | 100 | 0 | 0 | 0 | 0 |
| BRCOC2012386 | 11.403 | 0 | 40.395 | 0 | 0 | 0 | 0 |
| BRCOC2012551 | 7.213 | 0 | 25.551 | 0 | 0 | 0 | 15.997 |
| BRCOC2012813 | 0 | 39.306 | 60.694 | 0 | 0 | 0 | 0 |
| BRCOC2013448 | 0 | 0 | 100 | 0 | 0 | 0 | 0 |
| BRCOC2013573 | 0 | 0 | 100 | 0 | 0 | 0 | 0 |
| BRCOC2013675 | 0 | 0 | 100 | 0 | 0 | 0 | 0 |
| BRCOC2013780 | 0 | 0 | 49.505 | 0 | 0 | 0 | 0 |
| BRCOC2014013 | 0 | 0 | 19.212 | 0 | 0 | 0 | 0 |
| BRCOC2014033 | 15.633 | 0 | 55.382 | 15.950 | 0 | 0 | 0 |
| BRCOC2014400 | 0 | 0 | 59.512 | 0 | 0 | 0 | 0 |
| BRCOC2014748 | 0 | 0 | 100 | 0 | 0 | 0 | 0 |
| BRCOC2014833 | 0 | 0 | 100 | 0 | 0 | 0 | 0 |
| BRCOC2015597 | 0 | 0 | 74.813 | 0 | 0 | 0 | 0 |
| BRCOC2015824 | 0 | 0 | 100 | 0 | 0 | 0 | 0 |
| BRCOC2015908 | 0 | 0 | 67.066 | 0 | 0 | 0 | 0 |
| BRCOC2015944 | 0 | 0 | 100 | 0 | 0 | 0 | 0 |
| BRCOC2016525 | 0 | 0 | 100 | 0 | 0 | 0 | 0 |
| BRCOC2016661 | 0 | 0 | 100 | 0 | 0 | 0 | 0 |
| BRCOC2017061 | 0 | 0 | 100 | 0 | 0 | 0 | 0 |
| BRCOC2017652 | 0 | 0 | 48.902 | 0 | 0 | 0 | 0 |
| BRCOC2017856 | 0 | 0 | 100 | 0 | 0 | 0 | 0 |
| BRCOC2018775 | 0 | 0 | 35.892 | 0 | 0 | 0 | 0 |
| BRCOC2018877 | 0 | 0 | 67.066 | 0 | 0 | 0 | 0 |
| BRCOC2019255 | 0 | 39.250 | 30.303 | 0 | 0 | 0 | 0 |
| BRCOC2019549 | 0 | 0 | 100 | 0 | 0 | 0 | 0 |
| BRCOC2019841 | 0 | 0 | 83.309 | 0 | 0 | 0 | 0 |
| BRCOC2019896 | 0 | 0 | 77.856 | 0 | 0 | 0 | 0 |
| BRCOC2019903 | 0 | 0 | 56.781 | 0 | 0 | 0 | 0 |
| BRCOC2019910 | 36.872 | 0 | 4.428 | 3.188 | 4.652 | 4.507 | 6.930 |
| BRCOC2019934 | 0 | 0 | 100 | 0 | 0 | 0 | 0 |
| BRCOC2020122 | 0 | 0 | 100 | 0 | 0 | 0 | 0 |
| BRCOC2020142 | 22.014 | 0 | 77.986 | 0 | 0 | 0 | 0 |
| BRHIP1000058 | 0 | 0 | 0 | 100 | 0 | 0 | 0 |
| BRHIP1000072 | 0 | 0 | 0 | 100 | 0 | 0 | 0 |
| BRHIP1000104 | 3.922 | 14.996 | 13.894 | 4.001 | 7.299 | 4.714 | 5.074 |
| BRHIP1000108 | 12.929 | 0 | 0 | 17.587 | 0 | 0 | 9.558 |
| BRHIP1000129 | 0 | 0 | 0 | 50.312 | 0 | 0 | 0 |
| BRHIP1000174 | 0 | 0 | 17.961 | 5.173 | 0 | 18.281 | 5.622 |
| BRHIP1000258 | 25.855 | 0 | 0 | 26.379 | 0 | 0 | 0 |
| BRHIP2000021 | 0 | 0 | 0 | 11.573 | 0 | 0 | 0 |
| BRHIP2000079 | 0 | 0 | 0 | 100 | 0 | 0 | 0 |
| BRHIP2000087 | 0 | 0 | 0 | 13.429 | 24.496 | 0 | 7.299 |
| BRHIP2000163 | 37.398 | 0 | 0 | 38.155 | 0 | 0 | 0 |
| BRHIP2000210 | 0 | 0 | 0 | 58.973 | 0 | 0 | 0 |
| BRHIP2000239 | 0 | 0 | 0 | 47.666 | 0 | 0 | 0 |
| BRHIP2000245 | 0 | 0 | 0 | 55.026 | 0 | 0 | 0 |
| BRHIP2000312 | 32.424 | 0 | 0 | 13.232 | 0 | 0 | 14.383 |
| BRHIP2000359 | 0 | 0 | 0 | 100 | 0 | 0 | 0 |
| BRHIP2000445 | 0 | 0 | 0 | 100 | 0 | 0 | 0 |
| BRHIP2000506 | 0 | 0 | 0 | 100 | 0 | 0 | 0 |
| BRHIP2000532 | 0 | 0 | 0 | 100 | 0 | 0 | 0 |
| BRHIP2000534 | 0.342 | 0 | 0 | 0.349 | 0 | 0 | 0 |
| BRHIP2000553 | 2.570 | 0 | 0 | 5.243 | 0 | 0 | 2.850 |
| BRHIP2000621 | 0 | 0 | 0 | 100 | 0 | 0 | 0 |
| BRHIP2000691 | 0 | 0 | 0 | 100 | 0 | 0 | 0 |
| BRHIP2000819 | 0 | 0 | 0 | 2.204 | 0 | 0 | 4.791 |
| BRHIP2000826 | 0 | 0 | 0 | 100 | 0 | 0 | 0 |
| BRHIP2000920 | 36.630 | 0 | 0 | 37.371 | 0 | 0 | 0 |
| BRHIP2001074 | 0 | 0 | 0 | 39.976 | 0 | 0 | 0 |
| BRHIP2001099 | 0 | 0 | 0 | 7.072 | 0 | 0 | 0 |
| BRHIP2001686 | 0 | 0 | 0 | 100 | 0 | 0 | 0 |
| BRHIP2001782 | 0 | 0 | 0 | 100 | 0 | 0 | 0 |
| BRHIP2001802 | 0 | 0 | 0 | 100 | 0 | 0 | 0 |
| BRHIP2001805 | 0 | 0 | 0 | 14.757 | 0 | 0 | 0 |
| BRHIP2001886 | 0 | 0 | 0 | 50.312 | 0 | 0 | 0 |
| BRHIP2001927 | 0 | 0 | 0 | 100 | 0 | 0 | 0 |
| BRHIP2002122 | 0 | 0 | 0 | 100 | 0 | 0 | 0 |
| BRHIP2002143 | 0 | 0 | 0 | 58.973 | 0 | 0 | 0 |
| BRHIP2002172 | 0 | 0 | 0 | 100 | 0 | 0 | 0 |
| BRHIP2002339 | 7.848 | 0 | 0 | 24.020 | 0 | 0 | 8.703 |
| BRHIP2002346 | 0 | 0 | 0 | 100 | 0 | 0 | 0 |
| BRHIP2002510 | 0 | 0 | 0 | 100 | 0 | 0 | 0 |
| BRHIP2002664 | 0 | 0 | 0 | 100 | 0 | 0 | 0 |
| BRHIP2002722 | 0 | 0 | 0 | 100 | 0 | 0 | 0 |
| BRHIP2002929 | 0 | 0 | 0 | 100 | 0 | 0 | 0 |
| BRHIP2003048 | 0 | 0 | 0 | 100 | 0 | 0 | 0 |
| BRHIP2003062 | 18.998 | 0 | 0 | 9.691 | 0 | 0 | 0 |
| BRHIP2003242 | 0 | 0 | 0 | 100 | 0 | 0 | 0 |
| BRHIP2003272 | 0 | 0 | 0 | 100 | 0 | 0 | 0 |
| BRHIP2003748 | 0 | 0 | 0 | 100 | 0 | 0 | 0 |
| BRHIP2003786 | 0 | 0 | 0 | 100 | 0 | 0 | 0 |
| BRHIP2003828 | 0 | 0 | 0 | 100 | 0 | 0 | 0 |
| BRHIP2003917 | 0 | 0 | 0 | 11.981 | 0 | 0 | 0 |
| BRHIP2004312 | 0 | 0 | 0 | 100 | 0 | 0 | 0 |
| BRHIP2004355 | 0 | 0 | 0 | 100 | 0 | 0 | 0 |
| BRHIP2004359 | 0 | 0 | 0 | 24.839 | 0 | 0 | 0 |
| BRHIP2004814 | 0 | 0 | 0 | 35.391 | 0 | 0 | 0 |
| BRHIP2004883 | 0 | 0 | 0 | 100 | 0 | 0 | 0 |
| BRHIP2004902 | 0 | 0 | 0 | 100 | 0 | 0 | 0 |
| BRHIP2005010 | 15.764 | 0 | 0 | 16.084 | 0 | 0 | 0 |
| BRHIP2005236 | 0 | 0 | 0 | 100 | 0 | 0 | 0 |
| BRHIP2005271 | 7.083 | 0 | 0 | 14.453 | 0 | 0 | 7.855 |
| BRHIP2005354 | 0 | 0 | 0 | 100 | 0 | 0 | 0 |
| BRHIP2005600 | 0 | 0 | 0 | 100 | 0 | 0 | 0 |
| BRHIP2005719 | 49.499 | 0 | 0 | 50.501 | 0 | 0 | 0 |
| BRHIP2005724 | 0 | 0 | 0 | 100 | 0 | 0 | 0 |
| BRHIP2005752 | 0 | 0 | 0 | 58.973 | 0 | 0 | 0 |
| BRHIP2005932 | 0 | 0 | 0 | 100 | 0 | 0 | 0 |
| BRHIP2006617 | 0 | 0 | 0 | 100 | 0 | 0 | 0 |
| BRHIP2006782 | 0 | 0 | 0 | 100 | 0 | 0 | 0 |
| BRHIP2006800 | 0 | 0 | 0 | 100 | 0 | 0 | 0 |
| BRHIP2006819 | 0 | 0 | 0 | 100 | 0 | 0 | 0 |
| BRHIP2006921 | 0 | 0 | 0 | 100 | 0 | 0 | 0 |
| BRHIP2007305 | 1.132 | 0 | 0 | 1.155 | 0 | 0 | 0 |
| BRHIP2007307 | 0 | 0 | 0 | 29.041 | 0 | 0 | 0 |
| BRHIP2007616 | 0 | 0 | 0 | 100 | 0 | 0 | 0 |
| BRHIP2007690 | 0 | 0 | 0 | 100 | 0 | 0 | 0 |
| BRHIP2007741 | 6.943 | 0 | 0 | 3.542 | 0 | 0 | 5.775 |
| BRHIP2007928 | 0 | 0 | 0 | 100 | 0 | 0 | 0 |
| BRHIP2007969 | 0 | 0 | 0 | 100 | 0 | 0 | 0 |
| BRHIP2008269 | 0 | 0 | 0 | 28.389 | 0 | 0 | 0 |
| BRHIP2008389 | 0 | 0 | 0 | 100 | 0 | 0 | 0 |
| BRHIP2008607 | 17.957 | 0 | 0 | 36.640 | 0 | 0 | 19.913 |
| BRHIP2008756 | 0 | 0 | 0 | 100 | 0 | 0 | 0 |
| BRHIP2008909 | 0 | 0 | 0 | 100 | 0 | 0 | 0 |
| BRHIP2009019 | 0 | 0 | 0 | 58.973 | 0 | 0 | 0 |
| BRHIP2009177 | 0 | 0 | 0 | 100 | 0 | 0 | 0 |
| BRHIP2009340 | 0 | 0 | 0 | 100 | 0 | 0 | 0 |
| BRHIP2009351 | 0 | 0 | 0 | 100 | 0 | 0 | 0 |
| BRHIP2009414 | 0 | 0 | 0 | 100 | 0 | 0 | 0 |
| BRHIP2009474 | 0 | 0 | 0 | 47.917 | 0 | 0 | 52.083 |
| BRHIP2009617 | 0 | 0 | 0 | 100 | 0 | 0 | 0 |
| BRHIP2009685 | 7.124 | 0 | 0 | 7.269 | 0 | 0 | 7.901 |
| BRHIP2010217 | 0 | 0 | 0 | 11.396 | 0 | 0 | 18.580 |
| BRHIP2010309 | 0 | 0 | 0 | 100 | 0 | 0 | 0 |
| BRHIP2010444 | 0 | 0 | 0 | 47.917 | 0 | 0 | 52.083 |
| BRHIP2010487 | 0 | 0 | 0 | 4.196 | 0 | 0 | 0 |
| BRHIP2010571 | 0 | 0 | 0 | 31.420 | 0 | 0 | 0 |
| BRHIP2010593 | 0 | 0 | 0 | 3.573 | 0 | 0 | 0 |
| BRHIP2010610 | 0 | 0 | 0 | 100 | 0 | 0 | 0 |
| BRHIP2010723 | 0 | 0 | 0 | 50.312 | 0 | 0 | 0 |
| BRHIP2010744 | 0 | 0 | 0 | 100 | 0 | 0 | 0 |
| BRHIP2011080 | 0 | 0 | 0 | 35.577 | 0 | 0 | 0 |
| BRHIP2011120 | 15.351 | 0 | 0 | 15.661 | 28.568 | 0 | 0 |
| BRHIP2011199 | 0 | 0 | 0 | 55.026 | 0 | 0 | 0 |
| BRHIP2011491 | 0 | 0 | 0 | 0.843 | 0 | 8.941 | 0 |
| BRHIP2011508 | 0 | 0 | 0 | 22.032 | 0 | 0 | 23.948 |
| BRHIP2011576 | 0 | 0 | 0 | 19.559 | 0 | 0 | 0 |
| BRHIP2011616 | 0 | 0 | 0 | 46.104 | 0 | 0 | 0 |
| BRHIP2011783 | 0 | 0 | 0 | 100 | 0 | 0 | 0 |
| BRHIP2011838 | 0 | 0 | 0 | 100 | 0 | 0 | 0 |
| BRHIP2011891 | 0 | 0 | 0 | 60.949 | 0 | 0 | 0 |
| BRHIP2011933 | 0 | 0 | 0 | 100 | 0 | 0 | 0 |
| BRHIP2012054 | 0 | 0 | 0 | 29.041 | 0 | 0 | 0 |
| BRHIP2012059 | 0 | 0 | 0 | 100 | 0 | 0 | 0 |
| BRHIP2012141 | 0 | 0 | 0 | 47.917 | 0 | 0 | 52.083 |
| BRHIP2012314 | 0 | 0 | 0 | 100 | 0 | 0 | 0 |
| BRHIP2012360 | 0 | 0 | 0 | 100 | 0 | 0 | 0 |
| BRHIP2012545 | 0 | 0 | 0 | 100 | 0 | 0 | 0 |
| BRHIP2012580 | 0 | 0 | 0 | 100 | 0 | 0 | 0 |
| BRHIP2012627 | 0 | 0 | 0 | 16.026 | 0 | 0 | 0 |
| BRHIP2012642 | 0 | 0 | 0 | 100 | 0 | 0 | 0 |
| BRHIP2012750 | 0 | 0 | 0 | 100 | 0 | 0 | 0 |
| BRHIP2012922 | 0 | 0 | 0 | 100 | 0 | 0 | 0 |
| BRHIP2012923 | 0 | 69.219 | 0 | 30.781 | 0 | 0 | 0 |
| BRHIP2012972 | 0 | 0 | 0 | 47.917 | 0 | 0 | 52.083 |
| BRHIP2013286 | 0 | 0 | 0 | 17.468 | 0 | 0 | 0 |
| BRHIP2013447 | 0 | 0 | 0 | 100 | 0 | 0 | 0 |
| BRHIP2013510 | 0 | 0 | 0 | 100 | 0 | 0 | 0 |
| BRHIP2013699 | 0 | 0 | 0 | 21.770 | 0 | 0 | 23.663 |
| BRHIP2013723 | 0 | 0 | 0 | 100 | 0 | 0 | 0 |
| BRHIP2013958 | 0 | 0 | 0 | 36.661 | 0 | 0 | 39.849 |
| BRHIP2013972 | 0 | 0 | 0 | 100 | 0 | 0 | 0 |
| BRHIP2014063 | 0 | 0 | 0 | 36.967 | 0 | 0 | 0 |
| BRHIP2014228 | 29.439 | 0 | 0 | 15.017 | 0 | 0 | 0 |
| BRHIP2014285 | 0 | 0 | 0 | 100 | 0 | 0 | 0 |
| BRHIP2014291 | 0 | 0 | 0 | 100 | 0 | 0 | 0 |
| BRHIP2014373 | 0 | 0 | 0 | 34.432 | 0 | 0 | 37.426 |
| BRHIP2014386 | 0 | 0 | 0 | 55.026 | 0 | 0 | 0 |
| BRHIP2014387 | 0 | 0 | 0 | 100 | 0 | 0 | 0 |
| BRHIP2014954 | 0 | 0 | 0 | 100 | 0 | 0 | 0 |
| BRHIP2015153 | 20.038 | 0 | 0 | 20.443 | 0 | 0 | 0 |
| BRHIP2015224 | 22.746 | 0 | 0 | 13.924 | 0 | 0 | 20.179 |
| BRHIP2015245 | 14.244 | 0 | 0 | 19.377 | 0 | 0 | 13.164 |
| BRHIP2015356 | 0 | 0 | 0 | 100 | 0 | 0 | 0 |
| BRHIP2015360 | 0 | 0 | 0 | 100 | 0 | 0 | 0 |
| BRHIP2015679 | 2.509 | 0 | 0 | 5.119 | 0 | 0 | 0 |
| BRHIP2016005 | 0 | 0 | 0 | 100 | 0 | 0 | 0 |
| BRHIP2016125 | 0 | 0 | 0 | 100 | 0 | 0 | 0 |
| BRHIP2016788 | 0 | 0 | 0 | 100 | 0 | 0 | 0 |
| BRHIP2016968 | 0 | 0 | 0 | 100 | 0 | 0 | 0 |
| BRHIP2016990 | 0 | 0 | 0 | 100 | 0 | 0 | 0 |
| BRHIP2017010 | 0 | 0 | 0 | 14.025 | 0 | 0 | 0 |
| BRHIP2017315 | 0 | 0 | 0 | 100 | 0 | 0 | 0 |
| BRHIP2017385 | 0 | 0 | 0 | 100 | 0 | 0 | 0 |
| BRHIP2017404 | 0 | 0 | 0 | 100 | 0 | 0 | 0 |
| BRHIP2017542 | 0 | 0 | 0 | 100 | 0 | 0 | 0 |
| BRHIP2017553 | 0 | 0 | 0 | 100 | 0 | 0 | 0 |
| BRHIP2017642 | 12.915 | 0 | 0 | 26.353 | 0 | 0 | 0 |
| BRHIP2017714 | 0 | 0 | 0 | 100 | 0 | 0 | 0 |
| BRHIP2017780 | 0 | 0 | 0 | 100 | 0 | 0 | 0 |
| BRHIP2017945 | 0 | 0 | 0 | 3.288 | 0 | 0 | 0 |
| BRHIP2018014 | 0 | 0 | 0 | 100 | 0 | 0 | 0 |
| BRHIP2018369 | 0 | 0 | 0 | 100 | 0 | 0 | 0 |
| BRHIP2018612 | 0 | 0 | 0 | 100 | 0 | 0 | 0 |
| BRHIP2018635 | 0 | 0 | 0 | 60.949 | 0 | 0 | 0 |
| BRHIP2018650 | 0 | 0 | 0 | 100 | 0 | 0 | 0 |
| BRHIP2018712 | 4.050 | 0 | 0 | 4.132 | 0 | 0 | 0 |
| BRHIP2018719 | 0 | 0 | 0 | 50.312 | 0 | 0 | 0 |
| BRHIP2018805 | 0 | 0 | 0 | 100 | 0 | 0 | 0 |
| BRHIP2018998 | 0 | 0 | 0 | 100 | 0 | 0 | 0 |
| BRHIP2019007 | 0 | 0 | 0 | 100 | 0 | 0 | 0 |
| BRHIP2019047 | 0 | 0 | 0 | 100 | 0 | 0 | 0 |
| BRHIP2019149 | 0 | 0 | 0 | 11.267 | 0 | 0 | 0 |
| BRHIP2019177 | 0 | 0 | 0 | 100 | 0 | 0 | 0 |
| BRHIP2019186 | 0 | 0 | 0 | 4.027 | 0 | 0 | 0 |
| BRHIP2019291 | 0 | 0 | 0 | 100 | 0 | 0 | 0 |
| BRHIP2019494 | 0 | 0 | 0 | 74.193 | 0 | 0 | 0 |
| BRHIP2019589 | 0 | 0 | 0 | 100 | 0 | 0 | 0 |
| BRHIP2019641 | 0 | 0 | 0 | 100 | 0 | 0 | 0 |
| BRHIP2019819 | 8.671 | 0 | 0 | 17.692 | 0 | 0 | 0 |
| BRHIP2019884 | 0 | 0 | 0 | 100 | 0 | 0 | 0 |
| BRHIP2019972 | 0 | 0 | 0 | 100 | 0 | 0 | 0 |
| BRHIP2020182 | 0 | 0 | 0 | 100 | 0 | 0 | 0 |
| BRHIP2020509 | 0 | 0 | 0 | 19.104 | 0 | 0 | 0 |
| BRHIP2020573 | 9.050 | 0 | 0 | 18.466 | 0 | 0 | 0 |
| BRHIP2020622 | 0 | 53.082 | 0 | 23.605 | 0 | 0 | 0 |
| BRHIP2020695 | 49.499 | 0 | 0 | 50.501 | 0 | 0 | 0 |
| BRHIP2020743 | 0 | 0 | 0 | 100 | 0 | 0 | 0 |
| BRHIP2020799 | 5.061 | 0 | 0 | 36.143 | 0 | 0 | 5.612 |
| BRHIP2020842 | 0 | 0 | 0 | 100 | 0 | 0 | 0 |
| BRHIP2020859 | 0 | 0 | 0 | 100 | 0 | 0 | 0 |
| BRHIP2020930 | 0 | 0 | 0 | 100 | 0 | 0 | 0 |
| BRHIP2021102 | 0 | 0 | 0 | 100 | 0 | 0 | 0 |
| BRHIP2021289 | 0 | 0 | 0 | 100 | 0 | 0 | 0 |
| BRHIP2021495 | 0 | 14.715 | 0 | 6.544 | 0 | 0 | 0 |
| BRHIP2021615 | 0 | 0 | 0 | 100 | 0 | 0 | 0 |
| BRHIP2021744 | 0 | 0 | 0 | 100 | 0 | 0 | 0 |
| BRHIP2021762 | 8.358 | 0 | 0 | 11.369 | 0 | 0 | 3.090 |
| BRHIP2021858 | 3.346 | 0 | 0 | 3.414 | 0 | 0 | 0 |
| BRHIP2021870 | 0 | 10.186 | 0 | 4.530 | 0 | 0 | 4.924 |
| BRHIP2021929 | 0 | 0 | 0 | 100 | 0 | 0 | 0 |
| BRHIP2022006 | 0 | 0 | 0 | 100 | 0 | 0 | 0 |
| BRHIP2022054 | 0 | 16.949 | 17.448 | 2.512 | 0 | 0 | 5.462 |
| BRHIP2022221 | 0 | 0 | 0 | 47.917 | 0 | 0 | 52.083 |
| BRHIP2022228 | 9.138 | 20.964 | 0 | 9.323 | 0 | 0 | 0 |
| BRHIP2022298 | 0 | 0 | 0 | 100 | 0 | 0 | 0 |
| BRHIP2022326 | 1.701 | 1.952 | 0 | 3.472 | 0 | 0 | 0 |
| BRHIP2022467 | 0 | 32.605 | 0 | 14.499 | 52.896 | 0 | 0 |
| BRHIP2022708 | 0 | 0 | 0 | 100 | 0 | 0 | 0 |
| BRHIP2022986 | 2.845 | 0 | 0 | 5.806 | 0 | 0 | 0 |
| BRHIP2023071 | 0 | 0 | 0 | 50.312 | 0 | 0 | 0 |
| BRHIP2023229 | 0 | 0 | 0 | 15.312 | 0 | 0 | 0 |
| BRHIP2023272 | 0 | 0 | 0 | 3.443 | 0 | 0 | 0 |
| BRHIP2023292 | 0 | 0 | 0 | 35.577 | 0 | 0 | 0 |
| BRHIP2023309 | 11.666 | 0 | 0 | 23.804 | 0 | 0 | 0 |
| BRHIP2023369 | 17.477 | 0 | 0 | 17.830 | 0 | 0 | 0 |
| BRHIP2023438 | 0 | 0 | 0 | 26.026 | 0 | 0 | 0 |
| BRHIP2023695 | 0 | 0 | 0 | 36.438 | 0 | 0 | 0 |
| BRHIP2023735 | 0 | 0 | 0 | 100 | 0 | 0 | 0 |
| BRHIP2023762 | 2.386 | 0 | 0 | 2.435 | 0 | 0 | 2.646 |
| BRHIP2023773 | 0 | 0 | 0 | 29.041 | 0 | 0 | 0 |
| BRHIP2023860 | 0 | 0 | 0 | 100 | 0 | 0 | 0 |
| BRHIP2023869 | 0 | 0 | 0 | 36.661 | 0 | 0 | 39.849 |
| BRHIP2023888 | 2.208 | 1.688 | 1.303 | 0.751 | 1.369 | 2.653 | 4.488 |
| BRHIP2024046 | 0 | 0 | 0 | 31.291 | 0 | 0 | 0 |
| BRHIP2024165 | 0 | 0 | 0 | 100 | 0 | 0 | 0 |
| BRHIP2024201 | 0 | 0 | 0 | 100 | 0 | 0 | 0 |
| BRHIP2024347 | 0 | 0 | 0 | 100 | 0 | 0 | 0 |
| BRHIP2024398 | 0 | 0 | 0 | 8.590 | 0 | 0 | 0 |
| BRHIP2024549 | 0 | 0 | 0 | 100 | 0 | 0 | 0 |
| BRHIP2024742 | 8.730 | 0 | 4.418 | 7.635 | 0 | 0 | 8.298 |
| BRHIP2024789 | 0 | 0 | 0 | 100 | 0 | 0 | 0 |
| BRHIP2024911 | 0 | 0 | 0 | 9.456 | 0 | 0 | 0 |
| BRHIP2024941 | 0 | 0 | 0 | 100 | 0 | 0 | 0 |
| BRHIP2025245 | 0 | 0 | 0 | 47.666 | 0 | 0 | 0 |
| BRHIP2025366 | 0 | 0 | 0 | 100 | 0 | 0 | 0 |
| BRHIP2025448 | 0 | 0 | 0 | 31.199 | 0 | 0 | 0 |
| BRHIP2025679 | 0 | 0 | 0 | 58.973 | 0 | 0 | 0 |
| BRHIP2025739 | 0 | 0 | 0 | 100 | 0 | 0 | 0 |
| BRHIP2025797 | 0 | 0 | 0 | 100 | 0 | 0 | 0 |
| BRHIP2026021 | 0 | 0 | 0 | 100 | 0 | 0 | 0 |
| BRHIP2026061 | 0 | 0 | 0 | 31.199 | 0 | 0 | 0 |
| BRHIP2026155 | 0 | 0 | 0 | 100 | 0 | 0 | 0 |
| BRHIP2026214 | 0 | 0 | 0 | 35.577 | 0 | 0 | 0 |
| BRHIP2026288 | 0 | 0 | 0 | 22.018 | 0 | 0 | 0 |
| BRHIP2026346 | 0 | 0 | 0 | 100 | 0 | 0 | 0 |
| BRHIP2026557 | 0 | 0 | 0 | 100 | 0 | 0 | 0 |
| BRHIP2026723 | 0 | 0 | 0 | 100 | 0 | 0 | 0 |
| BRHIP2026877 | 0 | 69.219 | 0 | 30.781 | 0 | 0 | 0 |
| BRHIP2027017 | 0 | 26.410 | 0 | 11.745 | 0 | 0 | 0 |
| BRHIP2027054 | 0 | 0 | 0 | 100 | 0 | 0 | 0 |
| BRHIP2027077 | 2.020 | 0 | 0 | 2.061 | 0 | 0 | 0 |
| BRHIP2027563 | 0 | 0 | 0 | 100 | 0 | 0 | 0 |
| BRHIP2027685 | 0 | 0 | 0 | 50.312 | 0 | 0 | 0 |
| BRHIP2027762 | 0 | 0 | 0 | 100 | 0 | 0 | 0 |
| BRHIP2028187 | 0 | 0 | 0 | 74.193 | 0 | 0 | 0 |
| BRHIP2028303 | 0 | 0 | 0 | 100 | 0 | 0 | 0 |
| BRHIP2028330 | 0 | 0 | 0 | 100 | 0 | 0 | 0 |
| BRHIP2028480 | 0 | 0 | 0 | 100 | 0 | 0 | 0 |
| BRHIP2028583 | 0 | 0 | 0 | 12.691 | 0 | 0 | 0 |
| BRHIP2028593 | 0 | 0 | 0 | 100 | 0 | 0 | 0 |
| BRHIP2029176 | 0 | 0 | 0 | 100 | 0 | 0 | 0 |
| BRHIP2029283 | 0 | 0 | 0 | 100 | 0 | 0 | 0 |
| BRHIP2029393 | 0 | 0 | 0 | 100 | 0 | 0 | 0 |
| BRHIP2029529 | 0 | 0 | 0 | 100 | 0 | 0 | 0 |
| BRHIP2029643 | 0 | 0 | 0 | 15.240 | 55.599 | 0 | 0 |
| BRHIP2029663 | 0 | 0 | 0 | 45.093 | 0 | 0 | 0 |
| BRHIP3000017 | 8.819 | 0 | 15.620 | 4.499 | 0 | 0 | 0 |
| BRHIP3000060 | 0 | 0 | 0 | 47.917 | 0 | 0 | 52.083 |
| BRHIP3000070 | 0 | 0 | 0 | 100 | 0 | 0 | 0 |
| BRHIP3000081 | 0 | 0 | 0 | 100 | 0 | 0 | 0 |
| BRHIP3000111 | 0 | 2.349 | 3.627 | 3.133 | 3.810 | 3.691 | 3.406 |
| BRHIP3000131 | 7.345 | 0 | 13.009 | 14.986 | 0 | 0 | 12.217 |
| BRHIP3000240 | 0 | 0 | 0 | 32.393 | 0 | 0 | 0 |
| BRHIP3000377 | 0 | 0 | 0 | 5.877 | 0 | 0 | 6.388 |
| BRHIP3000457 | 0 | 0 | 0 | 27.708 | 0 | 0 | 0 |
| BRHIP3000475 | 0 | 0 | 0 | 22.059 | 0 | 0 | 0 |
| BRHIP3000488 | 0 | 0 | 0 | 100 | 0 | 0 | 0 |
| BRHIP3000526 | 0 | 0 | 0 | 100 | 0 | 0 | 0 |
| BRHIP3000626 | 0 | 0 | 0 | 100 | 0 | 0 | 0 |
| BRHIP3000859 | 0 | 0 | 0 | 100 | 0 | 0 | 0 |
| BRHIP3000907 | 0 | 0 | 0 | 100 | 0 | 0 | 0 |
| BRHIP3001076 | 0 | 0 | 0 | 46.104 | 0 | 0 | 0 |
| BRHIP3001079 | 0 | 0 | 0 | 100 | 0 | 0 | 0 |
| BRHIP3001141 | 0 | 0 | 0 | 100 | 0 | 0 | 0 |
| BRHIP3001283 | 0 | 0 | 0 | 50.312 | 0 | 0 | 0 |
| BRHIP3001338 | 0 | 0 | 0 | 100 | 0 | 0 | 0 |
| BRHIP3001360 | 0 | 0 | 0 | 58.973 | 0 | 0 | 0 |
| BRHIP3001481 | 0 | 0 | 0 | 38.435 | 0 | 0 | 0 |
| BRHIP3001878 | 0 | 0 | 0 | 100 | 0 | 0 | 0 |
| BRHIP3001964 | 0 | 0 | 0 | 100 | 0 | 0 | 0 |
| BRHIP3002114 | 0 | 0 | 0 | 39.793 | 0 | 0 | 0 |
| BRHIP3002124 | 0 | 0 | 0 | 100 | 0 | 0 | 0 |
| BRHIP3002141 | 0 | 0 | 0 | 16.008 | 0 | 0 | 0 |
| BRHIP3002363 | 0 | 0 | 0 | 100 | 0 | 0 | 0 |
| BRHIP3002691 | 0 | 0 | 0 | 16.923 | 0 | 0 | 18.395 |
| BRHIP3002920 | 0 | 0 | 0 | 64.789 | 0 | 0 | 35.211 |
| BRHIP3002931 | 0 | 0 | 0 | 100 | 0 | 0 | 0 |
| BRHIP3003063 | 10.960 | 0 | 0 | 55.910 | 0 | 0 | 0 |
| BRHIP3003126 | 0 | 0 | 0 | 74.193 | 0 | 0 | 0 |
| BRHIP3003306 | 0 | 0 | 0 | 60.949 | 0 | 0 | 0 |
| BRHIP3003340 | 0 | 0 | 0 | 100 | 0 | 0 | 0 |
| BRHIP3003395 | 0 | 0 | 0 | 100 | 0 | 0 | 0 |
| BRHIP3003484 | 0 | 0 | 0 | 100 | 0 | 0 | 0 |
| BRHIP3003644 | 0 | 0 | 0 | 100 | 0 | 0 | 0 |
| BRHIP3003688 | 0 | 0 | 0 | 100 | 0 | 0 | 0 |
| BRHIP3003795 | 0 | 0 | 0 | 45.093 | 0 | 0 | 0 |
| BRHIP3003961 | 0 | 0 | 0 | 19.351 | 0 | 0 | 21.034 |
| BRHIP3003984 | 27.477 | 0 | 0 | 28.033 | 0 | 0 | 15.236 |
| BRHIP3004193 | 0 | 0 | 0 | 100 | 0 | 0 | 0 |
| BRHIP3004215 | 0 | 0 | 0 | 13.851 | 0 | 0 | 15.055 |
| BRHIP3004416 | 0 | 0 | 0 | 100 | 0 | 0 | 0 |
| BRHIP3004710 | 0 | 0 | 0 | 100 | 0 | 0 | 0 |
| BRHIP3004725 | 0 | 0 | 0 | 100 | 0 | 0 | 0 |
| BRHIP3004774 | 39.520 | 0 | 0 | 60.480 | 0 | 0 | 0 |
| BRHIP3004786 | 0 | 0 | 0 | 100 | 0 | 0 | 0 |
| BRHIP3004863 | 0 | 0 | 0 | 100 | 0 | 0 | 0 |
| BRHIP3004968 | 0 | 0 | 0 | 34.325 | 0 | 0 | 0 |
| BRHIP3005037 | 0 | 0 | 0 | 82.401 | 0 | 0 | 0 |
| BRHIP3005137 | 0 | 0 | 0 | 100 | 0 | 0 | 0 |
| BRHIP3005142 | 0 | 0 | 0 | 60.949 | 0 | 0 | 0 |
| BRHIP3005231 | 0 | 0 | 0 | 100 | 0 | 0 | 0 |
| BRHIP3005307 | 0 | 0 | 0 | 46.104 | 0 | 0 | 0 |
| BRHIP3005574 | 0 | 0 | 0 | 43.125 | 0 | 0 | 46.875 |
| BRHIP3005673 | 0 | 0 | 0 | 52.564 | 0 | 0 | 0 |
| BRHIP3005768 | 0 | 0 | 0 | 47.917 | 0 | 0 | 52.083 |
| BRHIP3005801 | 0 | 0 | 0 | 19.121 | 0 | 67.577 | 0 |
| BRHIP3005944 | 0 | 0 | 0 | 100 | 0 | 0 | 0 |
| BRHIP3006279 | 0 | 0 | 0 | 100 | 0 | 0 | 0 |
| BRHIP3006294 | 0 | 0 | 0 | 40.683 | 0 | 0 | 0 |
| BRHIP3006390 | 49.499 | 0 | 0 | 50.501 | 0 | 0 | 0 |
| BRHIP3006449 | 0 | 0 | 0 | 100 | 0 | 0 | 0 |
| BRHIP3006683 | 24.100 | 0 | 0 | 49.175 | 0 | 0 | 26.726 |
| BRHIP3006717 | 15.318 | 0 | 0 | 10.419 | 6.335 | 0 | 5.662 |
| BRHIP3006786 | 0 | 0 | 0 | 100 | 0 | 0 | 0 |
| BRHIP3006950 | 0 | 0 | 0 | 100 | 0 | 0 | 0 |
| BRHIP3007172 | 0 | 0 | 0 | 100 | 0 | 0 | 0 |
| BRHIP3007183 | 0 | 31.449 | 0 | 13.985 | 0 | 0 | 45.605 |
| BRHIP3007195 | 0 | 0 | 0 | 75.737 | 0 | 0 | 0 |
| BRHIP3007223 | 2.016 | 0 | 0 | 2.057 | 0 | 0 | 4.472 |
| BRHIP3007239 | 0 | 0 | 0 | 100 | 0 | 0 | 0 |
| BRHIP3007291 | 0 | 0 | 0 | 100 | 0 | 0 | 0 |
| BRHIP3007409 | 31.957 | 0 | 0 | 32.604 | 0 | 0 | 35.439 |
| BRHIP3007424 | 0 | 0 | 0 | 100 | 0 | 0 | 0 |
| BRHIP3007483 | 0 | 0 | 0 | 100 | 0 | 0 | 0 |
| BRHIP3007586 | 17.255 | 0 | 0 | 24.645 | 0 | 0 | 11.481 |
| BRHIP3007609 | 0 | 0 | 0 | 100 | 0 | 0 | 0 |
| BRHIP3007640 | 0 | 0 | 0 | 68.316 | 0 | 0 | 0 |
| BRHIP3007960 | 0 | 0 | 0 | 100 | 0 | 0 | 0 |
| BRHIP3008082 | 0 | 0 | 0 | 45.093 | 0 | 0 | 0 |
| BRHIP3008183 | 0 | 0 | 0 | 100 | 0 | 0 | 0 |
| BRHIP3008314 | 0 | 0 | 0 | 100 | 0 | 0 | 0 |
| BRHIP3008320 | 23.349 | 0 | 0 | 47.644 | 0 | 0 | 0 |
| BRHIP3008344 | 0 | 0 | 0 | 46.104 | 0 | 0 | 0 |
| BRHIP3008405 | 35.187 | 0 | 0 | 17.949 | 0 | 0 | 0 |
| BRHIP3008565 | 0 | 0 | 0 | 46.104 | 0 | 0 | 0 |
| BRHIP3008598 | 0 | 0 | 0 | 100 | 0 | 0 | 0 |
| BRHIP3008606 | 0 | 0 | 0 | 100 | 0 | 0 | 0 |
| BRHIP3008691 | 49.499 | 0 | 0 | 50.501 | 0 | 0 | 0 |
| BRHIP3008714 | 0 | 0 | 0 | 100 | 0 | 0 | 0 |
| BRHIP3008997 | 0 | 0 | 0 | 100 | 0 | 0 | 0 |
| BRHIP3009099 | 0 | 0 | 0 | 35.577 | 0 | 0 | 0 |
| BRHIP3009140 | 0 | 0 | 0 | 100 | 0 | 0 | 0 |
| BRHIP3009181 | 0 | 0 | 0 | 100 | 0 | 0 | 0 |
| BRHIP3009318 | 0 | 0 | 0 | 100 | 0 | 0 | 0 |
| BRHIP3009427 | 0 | 0 | 0 | 100 | 0 | 0 | 0 |
| BRHIP3009448 | 0 | 0 | 0 | 100 | 0 | 0 | 0 |
| BRHIP3009672 | 0 | 0 | 0 | 100 | 0 | 0 | 0 |
| BRHIP3009753 | 0 | 0 | 0 | 1.187 | 0 | 0 | 0 |
| BRHIP3010135 | 0 | 0 | 0 | 100 | 0 | 0 | 0 |
| BRHIP3010289 | 0 | 0 | 0 | 100 | 0 | 0 | 0 |
| BRHIP3010529 | 0 | 0 | 0 | 100 | 0 | 0 | 0 |
| BRHIP3011082 | 0 | 0 | 0 | 100 | 0 | 0 | 0 |
| BRHIP3011241 | 0 | 0 | 0 | 100 | 0 | 0 | 0 |
| BRHIP3011269 | 0 | 0 | 0 | 100 | 0 | 0 | 0 |
| BRHIP3011460 | 0 | 0 | 0 | 100 | 0 | 0 | 0 |
| BRHIP3011567 | 0 | 0 | 0 | 100 | 0 | 0 | 0 |
| BRHIP3011831 | 0 | 0 | 0 | 100 | 0 | 0 | 0 |
| BRHIP3012185 | 0 | 0 | 0 | 100 | 0 | 0 | 0 |
| BRHIP3012289 | 0 | 0 | 0 | 100 | 0 | 0 | 0 |
| BRHIP3012357 | 0 | 0 | 0 | 100 | 0 | 0 | 0 |
| BRHIP3012736 | 0 | 0 | 0 | 33.611 | 0 | 0 | 0 |
| BRHIP3012745 | 0 | 0 | 0 | 100 | 0 | 0 | 0 |
| BRHIP3012868 | 0 | 0 | 0 | 100 | 0 | 0 | 0 |
| BRHIP3012997 | 0 | 0 | 0 | 2.837 | 0 | 0 | 0 |
| BRHIP3013078 | 0 | 0 | 0 | 100 | 0 | 0 | 0 |
| BRHIP3013271 | 0 | 0 | 0 | 100 | 0 | 0 | 0 |
| BRHIP3013588 | 0 | 0 | 0 | 60.949 | 0 | 0 | 0 |
| BRHIP3013698 | 0 | 0 | 0 | 100 | 0 | 0 | 0 |
| BRHIP3013765 | 0 | 0 | 0 | 100 | 0 | 0 | 0 |
| BRHIP3013897 | 0 | 0 | 0 | 100 | 0 | 0 | 0 |
| BRHIP3014237 | 0 | 0 | 0 | 100 | 0 | 0 | 0 |
| BRHIP3014675 | 37.925 | 0 | 0 | 19.346 | 0 | 0 | 0 |
| BRHIP3015620 | 0 | 0 | 0 | 100 | 0 | 0 | 0 |
| BRHIP3015751 | 0 | 0 | 0 | 100 | 0 | 0 | 0 |
| BRHIP3015854 | 0 | 0 | 0 | 100 | 0 | 0 | 0 |
| BRHIP3016032 | 0 | 0 | 0 | 100 | 0 | 0 | 0 |
| BRHIP3016213 | 0 | 0 | 0 | 100 | 0 | 0 | 0 |
| BRHIP3016302 | 0 | 0 | 0 | 100 | 0 | 0 | 0 |
| BRHIP3016421 | 0 | 0 | 0 | 100 | 0 | 0 | 0 |
| BRHIP3017109 | 0 | 0 | 0 | 100 | 0 | 0 | 0 |
| BRHIP3017146 | 0 | 0 | 0 | 66.943 | 0 | 0 | 0 |
| BRHIP3017256 | 0 | 0 | 0 | 100 | 0 | 0 | 0 |
| BRHIP3017325 | 0 | 0 | 0 | 100 | 0 | 0 | 0 |
| BRHIP3017558 | 0 | 0 | 0 | 100 | 0 | 0 | 0 |
| BRHIP3017637 | 0 | 0 | 0 | 100 | 0 | 0 | 0 |
| BRHIP3017855 | 15.316 | 0 | 0 | 11.720 | 0 | 0 | 4.246 |
| BRHIP3018278 | 0 | 0 | 0 | 39.793 | 0 | 0 | 0 |
| BRHIP3018784 | 6.930 | 0 | 12.276 | 3.535 | 0 | 0 | 0 |
| BRHIP3019643 | 0 | 0 | 0 | 100 | 0 | 0 | 0 |
| BRHIP3019824 | 0 | 0 | 0 | 100 | 0 | 0 | 0 |
| BRHIP3019880 | 0 | 0 | 0 | 100 | 0 | 0 | 0 |
| BRHIP3019956 | 0 | 0 | 0 | 100 | 0 | 0 | 0 |
| BRHIP3020046 | 0 | 0 | 0 | 2.067 | 3.770 | 0 | 1.123 |
| BRHIP3020155 | 0 | 0 | 0 | 100 | 0 | 0 | 0 |
| BRHIP3020182 | 0 | 0 | 0 | 100 | 0 | 0 | 0 |
| BRHIP3020733 | 0 | 0 | 0 | 55.026 | 0 | 0 | 0 |
| BRHIP3021019 | 7.745 | 0 | 0 | 7.901 | 0 | 0 | 0 |
| BRHIP3021499 | 0 | 0 | 0 | 100 | 0 | 0 | 0 |
| BRHIP3021534 | 0 | 0 | 0 | 100 | 0 | 0 | 0 |
| BRHIP3021778 | 0 | 0 | 0 | 100 | 0 | 0 | 0 |
| BRHIP3021987 | 0 | 0 | 0 | 36.438 | 0 | 0 | 0 |
| BRHIP3022656 | 0 | 0 | 0 | 100 | 0 | 0 | 0 |
| BRHIP3023922 | 0 | 0 | 0 | 100 | 0 | 0 | 0 |
| BRHIP3024118 | 0 | 0 | 0 | 100 | 0 | 0 | 0 |
| BRHIP3024533 | 0 | 0 | 0 | 100 | 0 | 0 | 0 |
| BRHIP3024703 | 0 | 0 | 0 | 100 | 0 | 0 | 0 |
| BRHIP3024725 | 0 | 0 | 0 | 100 | 0 | 0 | 0 |
| BRHIP3024820 | 0 | 0 | 0 | 100 | 0 | 0 | 0 |
| BRHIP3025161 | 0 | 0 | 0 | 100 | 0 | 0 | 0 |
| BRHIP3025702 | 0 | 0 | 0 | 100 | 0 | 0 | 0 |
| BRHIP3025795 | 0 | 0 | 0 | 100 | 0 | 0 | 0 |
| BRHIP3025844 | 0 | 0 | 0 | 100 | 0 | 0 | 0 |
| BRHIP3026052 | 0 | 0 | 0 | 45.860 | 0 | 0 | 0 |
| BRHIP3026097 | 0 | 0 | 0 | 100 | 0 | 0 | 0 |
| BRHIP3026231 | 0 | 0 | 0 | 100 | 0 | 0 | 0 |
| BRHIP3026335 | 0 | 0 | 0 | 100 | 0 | 0 | 0 |
| BRHIP3026651 | 0 | 0 | 0 | 100 | 0 | 0 | 0 |
| BRHIP3027105 | 49.499 | 0 | 0 | 50.501 | 0 | 0 | 0 |
| BRHIP3027137 | 0 | 0 | 0 | 100 | 0 | 0 | 0 |
| BRHIP3027160 | 0 | 0 | 0 | 100 | 0 | 0 | 0 |
| BRHIP3027191 | 0 | 0 | 0 | 100 | 0 | 0 | 0 |
| BRHIP3027501 | 0 | 0 | 0 | 100 | 0 | 0 | 0 |
| BRHIP3027594 | 19.439 | 0 | 0 | 19.832 | 0 | 0 | 21.557 |
| BRHIP3027651 | 0 | 0 | 0 | 100 | 0 | 0 | 0 |
| BRHIP3027854 | 0 | 0 | 0 | 100 | 0 | 0 | 0 |
| BRHIP3027947 | 0 | 0 | 0 | 100 | 0 | 0 | 0 |
| BRHIP3028570 | 31.957 | 0 | 0 | 32.604 | 0 | 0 | 35.439 |
| BRHIP3028742 | 0 | 0 | 0 | 100 | 0 | 0 | 0 |
| BRSSN1000056 | 0 | 0 | 0 | 0 | 22.550 | 0 | 6.719 |
| BRSSN1000059 | 0 | 0 | 0 | 0 | 50.093 | 0 | 0 |
| BRSSN1000092 | 0 | 0 | 0 | 0 | 100 | 0 | 0 |
| BRSSN1000153 | 0 | 0 | 0 | 0 | 100 | 0 | 0 |
| BRSSN2000097 | 0 | 0 | 0 | 0 | 25.771 | 0 | 0 |
| BRSSN2000175 | 0 | 0 | 0 | 0 | 100 | 0 | 0 |
| BRSSN2000197 | 0 | 0 | 0 | 0 | 81.697 | 0 | 0 |
| BRSSN2000244 | 0 | 0 | 0 | 0 | 50.137 | 0 | 0 |
| BRSSN2000293 | 0 | 7.523 | 23.234 | 3.346 | 36.616 | 23.648 | 0 |
| BRSSN2000295 | 6.926 | 15.889 | 0 | 0 | 25.777 | 0 | 7.680 |
| BRSSN2000312 | 10.759 | 0 | 0 | 0 | 40.045 | 0 | 0 |
| BRSSN2000498 | 4.377 | 0 | 3.876 | 1.116 | 12.218 | 0 | 1.213 |
| BRSSN2000518 | 0 | 0 | 0 | 0 | 100 | 0 | 0 |
| BRSSN2000561 | 0 | 0 | 0 | 0 | 23.338 | 0 | 13.907 |
| BRSSN2000566 | 4.864 | 0 | 0 | 9.925 | 18.104 | 0 | 5.394 |
| BRSSN2000684 | 23.433 | 0 | 0 | 19.126 | 17.444 | 0 | 10.395 |
| BRSSN2000715 | 0 | 0 | 0 | 0 | 100 | 0 | 0 |
| BRSSN2000832 | 12.039 | 0 | 0 | 24.566 | 44.810 | 0 | 0 |
| BRSSN2001213 | 0 | 0 | 0 | 0 | 75.732 | 0 | 0 |
| BRSSN2001275 | 0 | 38.134 | 0 | 0 | 61.866 | 0 | 0 |
| BRSSN2001342 | 0 | 0 | 0 | 0 | 100 | 0 | 0 |
| BRSSN2001426 | 0 | 0 | 0 | 0 | 100 | 0 | 0 |
| BRSSN2001503 | 0 | 0 | 0 | 0 | 100 | 0 | 0 |
| BRSSN2001579 | 0 | 0 | 0 | 0 | 50.137 | 0 | 0 |
| BRSSN2001758 | 0 | 21.568 | 0 | 0 | 34.990 | 0 | 0 |
| BRSSN2001810 | 0 | 0 | 0 | 0 | 100 | 0 | 0 |
| BRSSN2001869 | 0 | 6.925 | 0 | 0 | 11.235 | 0 | 6.695 |
| BRSSN2002857 | 0 | 0 | 0 | 0 | 100 | 0 | 0 |
| BRSSN2002859 | 0 | 0 | 0 | 0 | 100 | 0 | 0 |
| BRSSN2003012 | 0 | 0 | 0 | 0 | 16.101 | 0 | 0 |
| BRSSN2003086 | 0 | 0 | 0 | 0 | 100 | 0 | 0 |
| BRSSN2003093 | 0 | 0 | 0 | 0 | 100 | 0 | 0 |
| BRSSN2003820 | 0 | 0 | 0 | 0 | 100 | 0 | 0 |
| BRSSN2003841 | 0 | 0 | 0 | 0 | 100 | 0 | 0 |
| BRSSN2003937 | 0 | 0 | 0 | 0 | 100 | 0 | 0 |
| BRSSN2004142 | 0 | 0 | 0 | 0 | 100 | 0 | 0 |
| BRSSN2004303 | 0 | 0 | 0 | 0 | 100 | 0 | 0 |
| BRSSN2004304 | 0 | 0 | 0 | 0 | 100 | 0 | 0 |
| BRSSN2004496 | 0 | 0 | 0 | 0 | 78.696 | 0 | 0 |
| BRSSN2004657 | 0 | 0 | 0 | 0 | 85.061 | 0 | 0 |
| BRSSN2004679 | 0 | 0 | 0 | 0 | 56.646 | 0 | 16.878 |
| BRSSN2004686 | 0 | 0 | 0 | 0 | 100 | 0 | 0 |
| BRSSN2004710 | 0 | 0 | 0 | 0 | 100 | 0 | 0 |
| BRSSN2004719 | 0 | 12.020 | 0 | 10.691 | 39.002 | 18.892 | 0 |
| BRSSN2005847 | 0 | 0 | 0 | 0 | 81.697 | 0 | 0 |
| BRSSN2006133 | 0 | 0 | 0 | 0 | 100 | 0 | 0 |
| BRSSN2006390 | 0 | 0 | 0 | 0 | 100 | 0 | 0 |
| BRSSN2006575 | 0 | 0 | 0 | 0 | 100 | 0 | 0 |
| BRSSN2006611 | 0 | 0 | 0 | 0 | 78.696 | 0 | 0 |
| BRSSN2006644 | 0 | 0 | 0 | 0 | 100 | 0 | 0 |
| BRSSN2006892 | 0 | 0 | 0 | 0 | 57.631 | 0 | 0 |
| BRSSN2007076 | 0 | 0 | 0 | 0 | 100 | 0 | 0 |
| BRSSN2007168 | 0 | 0 | 0 | 0 | 100 | 0 | 0 |
| BRSSN2007202 | 0 | 38.134 | 0 | 0 | 61.866 | 0 | 0 |
| BRSSN2007338 | 0 | 0 | 0 | 0 | 27.509 | 0 | 0 |
| BRSSN2007464 | 0 | 0 | 0 | 0 | 15.231 | 0 | 0 |
| BRSSN2007504 | 0 | 0 | 0 | 0 | 100 | 0 | 0 |
| BRSSN2007819 | 0 | 0 | 0 | 0 | 100 | 0 | 0 |
| BRSSN2008125 | 0 | 0 | 0 | 0 | 100 | 0 | 0 |
| BRSSN2008419 | 0 | 0 | 0 | 0 | 100 | 0 | 0 |
| BRSSN2008464 | 0 | 0 | 0 | 0 | 100 | 0 | 0 |
| BRSSN2008549 | 0 | 0 | 0 | 17.436 | 63.611 | 0 | 18.953 |
| BRSSN2008797 | 0 | 0 | 0 | 0 | 77.045 | 0 | 22.955 |
| BRSSN2009119 | 0 | 0 | 0 | 0 | 68.148 | 0 | 0 |
| BRSSN2009378 | 0 | 0 | 0 | 0 | 100 | 0 | 0 |
| BRSSN2009389 | 0 | 0 | 0 | 0 | 100 | 0 | 0 |
| BRSSN2009395 | 0 | 0 | 0 | 0 | 100 | 0 | 0 |
| BRSSN2009518 | 1.722 | 0 | 6.102 | 1.757 | 6.411 | 0 | 0 |
| BRSSN2009627 | 0 | 0 | 0 | 0 | 100 | 0 | 0 |
| BRSSN2009630 | 0 | 0 | 0 | 0 | 100 | 0 | 0 |
| BRSSN2009702 | 0 | 0 | 0 | 0 | 100 | 0 | 0 |
| BRSSN2010019 | 0 | 0 | 0 | 0 | 43.197 | 0 | 0 |
| BRSSN2010110 | 0 | 0 | 0 | 0 | 100 | 0 | 0 |
| BRSSN2010587 | 0 | 0 | 0 | 0 | 83.985 | 0 | 0 |
| BRSSN2010596 | 0 | 0 | 0 | 0 | 44.612 | 0 | 0 |
| BRSSN2010830 | 0 | 0 | 0 | 0 | 100 | 0 | 0 |
| BRSSN2011262 | 0 | 0 | 0 | 0 | 10.489 | 0 | 0 |
| BRSSN2011653 | 0 | 0 | 0 | 0 | 18.443 | 0 | 0 |
| BRSSN2011738 | 31.553 | 0 | 0 | 0 | 39.146 | 0 | 0 |
| BRSSN2011843 | 0 | 0 | 0 | 0 | 44.546 | 0 | 0 |
| BRSSN2012081 | 0 | 0 | 0 | 0 | 100 | 0 | 0 |
| BRSSN2012103 | 0 | 0 | 0 | 0 | 100 | 0 | 0 |
| BRSSN2012157 | 0 | 0 | 0 | 0 | 100 | 0 | 0 |
| BRSSN2012198 | 0 | 0 | 0 | 0 | 100 | 0 | 0 |
| BRSSN2012254 | 0 | 0 | 0 | 0 | 100 | 0 | 0 |
| BRSSN2012439 | 0 | 0 | 0 | 0 | 81.697 | 0 | 0 |
| BRSSN2012622 | 0 | 0 | 0 | 0 | 100 | 0 | 0 |
| BRSSN2012691 | 0 | 0 | 0 | 0 | 100 | 0 | 0 |
| BRSSN2012838 | 0 | 0 | 0 | 0 | 100 | 0 | 0 |
| BRSSN2013095 | 0 | 0 | 0 | 21.514 | 78.486 | 0 | 0 |
| BRSSN2013544 | 0 | 0 | 0 | 0 | 100 | 0 | 0 |
| BRSSN2013696 | 0 | 0 | 0 | 0 | 13.293 | 0 | 0 |
| BRSSN2013702 | 0 | 0 | 33.606 | 0 | 35.308 | 0 | 0 |
| BRSSN2013733 | 0 | 0 | 0 | 0 | 78.696 | 0 | 0 |
| BRSSN2013753 | 0 | 0 | 0 | 0 | 54.884 | 0 | 0 |
| BRSSN2013874 | 0 | 0 | 0 | 0 | 100 | 0 | 0 |
| BRSSN2014112 | 0 | 0 | 0 | 0 | 100 | 0 | 0 |
| BRSSN2014294 | 0 | 0 | 0 | 0 | 34.652 | 0 | 0 |
| BRSSN2014299 | 3.695 | 0 | 0 | 3.770 | 13.753 | 0 | 0 |
| BRSSN2014424 | 0 | 38.134 | 0 | 0 | 61.866 | 0 | 0 |
| BRSSN2014513 | 0 | 0 | 0 | 0 | 100 | 0 | 0 |
| BRSSN2014556 | 0 | 0 | 0 | 0 | 100 | 0 | 0 |
| BRSSN2014573 | 0 | 0 | 0 | 0 | 100 | 0 | 0 |
| BRSSN2014610 | 0 | 0 | 0 | 10.927 | 13.288 | 0 | 11.878 |
| BRSSN2014636 | 0 | 0 | 0 | 0 | 100 | 0 | 0 |
| BRSSN2014685 | 0 | 0 | 0 | 0 | 52.851 | 0 | 0 |
| BRSSN2015199 | 0 | 0 | 0 | 0 | 83.985 | 0 | 0 |
| BRSSN2015238 | 10.270 | 0 | 0 | 6.985 | 12.742 | 0 | 0 |
| BRSSN2015369 | 0 | 0 | 0 | 0 | 100 | 0 | 0 |
| BRSSN2015493 | 0 | 0 | 0 | 0 | 13.605 | 0 | 0 |
| BRSSN2015497 | 0 | 0 | 0 | 0 | 26.619 | 0 | 7.931 |
| BRSSN2015554 | 0 | 0 | 48.765 | 0 | 51.235 | 0 | 0 |
| BRSSN2015707 | 0 | 0 | 0 | 0 | 100 | 0 | 0 |
| BRSSN2015773 | 0 | 0 | 0 | 0 | 100 | 0 | 0 |
| BRSSN2015907 | 0 | 0 | 0 | 0 | 100 | 0 | 0 |
| BRSSN2015982 | 0 | 0 | 0 | 0 | 60.467 | 0 | 0 |
| BRSSN2016589 | 0 | 0 | 0 | 0 | 100 | 0 | 0 |
| BRSSN2016768 | 0 | 0 | 0 | 0 | 100 | 0 | 0 |
| BRSSN2016905 | 0 | 0 | 0 | 0 | 100 | 0 | 0 |
| BRSSN2017297 | 0 | 0 | 0 | 0 | 100 | 0 | 0 |
| BRSSN2017422 | 0 | 0 | 0 | 29.338 | 53.514 | 0 | 0 |
| BRSSN2017530 | 0 | 0 | 0 | 0 | 100 | 0 | 0 |
| BRSSN2017682 | 0 | 0 | 0 | 0 | 100 | 0 | 0 |
| BRSSN2017757 | 0 | 0 | 0 | 0 | 100 | 0 | 0 |
| BRSSN2018218 | 0 | 0 | 0 | 0 | 100 | 0 | 0 |
| BRSSN2018440 | 0 | 0 | 0 | 0 | 74.975 | 0 | 0 |
| BRSSN2018447 | 19.606 | 0 | 0 | 13.335 | 24.324 | 0 | 0 |
| BRSSN2018581 | 0 | 0 | 0 | 0 | 50.137 | 0 | 0 |
| BRSSN2018731 | 0 | 0 | 0 | 0 | 100 | 0 | 0 |
| BRSSN2018771 | 0 | 0 | 0 | 0 | 100 | 0 | 0 |
| BRSSN2018925 | 0 | 0 | 0 | 0 | 100 | 0 | 0 |
| BRSTN1000083 | 0.638 | 0 | 2.262 | 1.303 | 0 | 2.302 | 0 |
| BRSTN1000097 | 0 | 0 | 0 | 0 | 0 | 100 | 0 |
| BRSTN2000058 | 0 | 33.205 | 25.637 | 0 | 8.978 | 17.396 | 0 |
| BRSTN2000070 | 0 | 0 | 0 | 22.055 | 0 | 77.945 | 0 |
| BRSTN2000220 | 0 | 7.871 | 0 | 10.501 | 0 | 37.113 | 15.219 |
| BRSTN2000312 | 0 | 0 | 0 | 0 | 0 | 100 | 0 |
| BRSTN2000367 | 0 | 0 | 0 | 0 | 0 | 84.653 | 0 |
| BRSTN2000536 | 0 | 0 | 0 | 22.055 | 0 | 77.945 | 0 |
| BRSTN2000872 | 0 | 0 | 0 | 0 | 0 | 100 | 0 |
| BRSTN2001067 | 0 | 0 | 0 | 0 | 0 | 44.646 | 0 |
| BRSTN2001613 | 0 | 0 | 0 | 0 | 0 | 100 | 0 |
| BRSTN2001801 | 0 | 0 | 0 | 0 | 0 | 100 | 0 |
| BRSTN2002105 | 0 | 0 | 0 | 0 | 0 | 100 | 0 |
| BRSTN2002400 | 0 | 0 | 0 | 0 | 0 | 100 | 0 |
| BRSTN2002532 | 0 | 0 | 0 | 0 | 0 | 100 | 0 |
| BRSTN2002691 | 0 | 0 | 0 | 0 | 0 | 100 | 0 |
| BRSTN2003590 | 0 | 0 | 0 | 0 | 0 | 100 | 0 |
| BRSTN2003835 | 0 | 0 | 0 | 0 | 0 | 100 | 0 |
| BRSTN2004863 | 0 | 0 | 35.295 | 0 | 0 | 35.924 | 11.049 |
| BRSTN2004869 | 0 | 0 | 0 | 0 | 0 | 76.298 | 0 |
| BRSTN2004987 | 0 | 0 | 0 | 0 | 0 | 100 | 0 |
| BRSTN2005721 | 0 | 0 | 0 | 0 | 0 | 100 | 0 |
| BRSTN2006306 | 0 | 0 | 0 | 0 | 0 | 100 | 0 |
| BRSTN2006466 | 0 | 0 | 0 | 0 | 0 | 100 | 0 |
| BRSTN2006580 | 0 | 0 | 0 | 0 | 0 | 83.553 | 0 |
| BRSTN2006583 | 0 | 0 | 0 | 0 | 0 | 76.479 | 23.521 |
| BRSTN2006638 | 0 | 0 | 0 | 0 | 0 | 100 | 0 |
| BRSTN2006865 | 0 | 0 | 0 | 0 | 0 | 100 | 0 |
| BRSTN2007000 | 0 | 0 | 0 | 0 | 0 | 100 | 0 |
| BRSTN2007118 | 0 | 0 | 0 | 0 | 0 | 100 | 0 |
| BRSTN2007284 | 0 | 38.885 | 0 | 0 | 0 | 61.115 | 0 |
| BRSTN2007765 | 0 | 0 | 0 | 0 | 0 | 100 | 0 |
| BRSTN2008052 | 32.844 | 0 | 0 | 0 | 0 | 39.475 | 12.141 |
| BRSTN2008283 | 0 | 0 | 0 | 0 | 0 | 100 | 0 |
| BRSTN2008367 | 0 | 0 | 0 | 0 | 0 | 100 | 0 |
| BRSTN2008418 | 0 | 0 | 0 | 0 | 0 | 100 | 0 |
| BRSTN2008457 | 0 | 0 | 0 | 0 | 0 | 100 | 0 |
| BRSTN2008475 | 0 | 0 | 0 | 0 | 0 | 100 | 0 |
| BRSTN2008930 | 0 | 0 | 0 | 0 | 0 | 100 | 0 |
| BRSTN2009247 | 0 | 0 | 0 | 0 | 0 | 100 | 0 |
| BRSTN2010089 | 6.598 | 0 | 23.372 | 0 | 0 | 23.789 | 0 |
| BRSTN2010107 | 1.338 | 13.047 | 16.591 | 5.802 | 11.205 | 6.031 | 8.16 |
| BRSTN2010363 | 0 | 0 | 0 | 0 | 0 | 100 | 0 |
| BRSTN2010416 | 0 | 0 | 0 | 0 | 0 | 100 | 0 |
| BRSTN2010500 | 0 | 0 | 0 | 0 | 0 | 100 | 0 |
| BRSTN2010569 | 0 | 0 | 0 | 0 | 0 | 100 | 0 |
| BRSTN2010750 | 0 | 0 | 0 | 0 | 0 | 36.677 | 0 |
| BRSTN2010923 | 0 | 0 | 0 | 0 | 0 | 100 | 0 |
| BRSTN2011211 | 0 | 0 | 0 | 0 | 0 | 100 | 0 |
| BRSTN2011597 | 0 | 0 | 0 | 0 | 0 | 83.553 | 0 |
| BRSTN2011688 | 0 | 0 | 0 | 0 | 0 | 100 | 0 |
| BRSTN2011799 | 0 | 0 | 0 | 0 | 0 | 100 | 0 |
| BRSTN2011961 | 0 | 0 | 1.026 | 0 | 1.078 | 1.044 | 1.285 |
| BRSTN2012069 | 0.047 | 0.391 | 0.101 | 0 | 0 | 0.853 | 0 |
| BRSTN2012089 | 5.800 | 0 | 0 | 0 | 0 | 20.915 | 6.432 |
| BRSTN2012174 | 0.202 | 0.928 | 0.717 | 0 | 1.506 | 3.646 | 0.224 |
| BRSTN2012284 | 0 | 0 | 0 | 0 | 0 | 100 | 0 |
| BRSTN2012320 | 0 | 0 | 0 | 0 | 0 | 100 | 0 |
| BRSTN2012380 | 0 | 0 | 0 | 0 | 0 | 100 | 0 |
| BRSTN2012451 | 0 | 0 | 0 | 0.843 | 0 | 5.956 | 0.916 |
| BRSTN2013171 | 0 | 15.900 | 0 | 0 | 0 | 12.495 | 0 |
| BRSTN2013354 | 0 | 0 | 0 | 0 | 0 | 100 | 0 |
| BRSTN2013502 | 0 | 0 | 0 | 0 | 0 | 17.527 | 2.695 |
| BRSTN2013931 | 2.899 | 0 | 0 | 0 | 0 | 3.484 | 2.143 |
| BRSTN2014490 | 0 | 0 | 0 | 0 | 0 | 33.221 | 0 |
| BRSTN2014633 | 0 | 0 | 0 | 0 | 0 | 100 | 0 |
| BRSTN2014751 | 0 | 0 | 0 | 0 | 0 | 100 | 0 |
| BRSTN2014770 | 0 | 0 | 0 | 0 | 0 | 100 | 0 |
| BRSTN2015015 | 14.017 | 0 | 0 | 0 | 0 | 50.541 | 15.544 |
| BRSTN2015889 | 0 | 0 | 0 | 3.952 | 0 | 13.967 | 8.591 |
| BRSTN2015978 | 1.881 | 14.547 | 14.724 | 3.002 | 19.309 | 3.719 | 0.370 |
| BRSTN2016066 | 0 | 0 | 0 | 0 | 0 | 100 | 0 |
| BRSTN2016335 | 0 | 0 | 0 | 0 | 0 | 100 | 0 |
| BRSTN2016470 | 0.438 | 1.004 | 0 | 0.447 | 0 | 5.524 | 0 |
| BRSTN2016678 | 0 | 0 | 0 | 0 | 0 | 33.084 | 0 |
| BRSTN2016892 | 0 | 0 | 0 | 0 | 0 | 10.759 | 0 |
| BRSTN2016918 | 0 | 0 | 0 | 0 | 0 | 100 | 0 |
| BRSTN2016992 | 1.476 | 3.387 | 1.743 | 2.510 | 5.495 | 15.970 | 1.091 |
| BRSTN2017104 | 0 | 8.946 | 0 | 0 | 7.257 | 7.030 | 0 |
| BRSTN2017151 | 0 | 0 | 0 | 0 | 0 | 100 | 0 |
| BRSTN2017237 | 0 | 0 | 0 | 0 | 0 | 100 | 0 |
| BRSTN2017379 | 0 | 0 | 0 | 0 | 0 | 100 | 0 |
| BRSTN2017761 | 0 | 0 | 0 | 0 | 0 | 76.298 | 0 |
| BRSTN2017771 | 0 | 0 | 0 | 0 | 0 | 100 | 0 |
| BRSTN2017995 | 0 | 0 | 0 | 0 | 0 | 100 | 0 |
| BRSTN2018077 | 0 | 38.885 | 0 | 0 | 0 | 61.115 | 0 |
| BRSTN2018083 | 0 | 0 | 0 | 22.055 | 0 | 77.945 | 0 |
| BRSTN2018596 | 0 | 0 | 0 | 0 | 0 | 100 | 0 |
| BRSTN2018692 | 0 | 0 | 0 | 0 | 0 | 100 | 0 |
| BRSTN2018712 | 0 | 0 | 0 | 0 | 0 | 100 | 0 |
| BRSTN2019079 | 0 | 0 | 0 | 0 | 0 | 100 | 0 |
| BRSTN2019129 | 0 | 0 | 0 | 0 | 0 | 100 | 0 |
| BRTHA1000021 | 17.174 | 39.401 | 0 | 0 | 0 | 0 | 19.045 |
| BRTHA1000063 | 0 | 0 | 0 | 0 | 0 | 0 | 100 |
| BRTHA1000311 | 11.803 | 6.770 | 0 | 6.021 | 10.982 | 0 | 9.817 |
| BRTHA2000057 | 0 | 0 | 0 | 0 | 0 | 0 | 48.181 |
| BRTHA2000474 | 0 | 0 | 0 | 0 | 0 | 0 | 100 |
| BRTHA2000514 | 0 | 0 | 0 | 0 | 0 | 0 | 100 |
| BRTHA2000574 | 0 | 0 | 0 | 0 | 0 | 0 | 100 |
| BRTHA2000855 | 0 | 0 | 0 | 0 | 0 | 0 | 100 |
| BRTHA2000969 | 0 | 0 | 0 | 0 | 0 | 0 | 100 |
| BRTHA2000972 | 0 | 0 | 0 | 0 | 0 | 0 | 100 |
| BRTHA2001304 | 0 | 0 | 0 | 0 | 0 | 0 | 100 |
| BRTHA2001370 | 0 | 0 | 0 | 0 | 0 | 0 | 100 |
| BRTHA2001462 | 0 | 0 | 0 | 0 | 0 | 0 | 100 |
| BRTHA2001717 | 0 | 0 | 0 | 0 | 0 | 0 | 100 |
| BRTHA2001812 | 0 | 0 | 0 | 0 | 0 | 30.730 | 9.451 |
| BRTHA2002091 | 0 | 0 | 0 | 0 | 0 | 0 | 100 |
| BRTHA2002115 | 0 | 0 | 0 | 0 | 0 | 0 | 100 |
| BRTHA2002133 | 0 | 0 | 0 | 0 | 0 | 0 | 100 |
| BRTHA2002281 | 0 | 0 | 0 | 0 | 0 | 0 | 100 |
| BRTHA2002376 | 0 | 0 | 0 | 0 | 0 | 0 | 100 |
| BRTHA2002442 | 0 | 0 | 0 | 31.507 | 0 | 0 | 68.493 |
| BRTHA2002493 | 0 | 0 | 0 | 0 | 0 | 0 | 100 |
| BRTHA2002565 | 0 | 0 | 0 | 0 | 0 | 0 | 100 |
| BRTHA2002608 | 0 | 0 | 0 | 0 | 0 | 0 | 100 |
| BRTHA2002702 | 0 | 0 | 0 | 0 | 0 | 0 | 100 |
| BRTHA2002721 | 0 | 0 | 0 | 0 | 0 | 0 | 100 |
| BRTHA2002726 | 0 | 0 | 0 | 0 | 0 | 0 | 23.821 |
| BRTHA2002741 | 0 | 0 | 0 | 0 | 0 | 0 | 100 |
| BRTHA2002808 | 0 | 0 | 0 | 0 | 0 | 0 | 12.857 |
| BRTHA2002896 | 0 | 0 | 0 | 0 | 0 | 0 | 100 |
| BRTHA2002903 | 0 | 0 | 0 | 0 | 0 | 0 | 100 |
| BRTHA2003030 | 0 | 0 | 0 | 0 | 0 | 0 | 100 |
| BRTHA2003110 | 0 | 0 | 0 | 0 | 0 | 0 | 100 |
| BRTHA2003116 | 0 | 0 | 0 | 0 | 0 | 0 | 100 |
| BRTHA2003436 | 0 | 0 | 0 | 0 | 0 | 0 | 100 |
| BRTHA2003759 | 0 | 0 | 0 | 0 | 0 | 0 | 100 |
| BRTHA2003794 | 0 | 0 | 0 | 0 | 0 | 0 | 100 |
| BRTHA2004361 | 0 | 0 | 0 | 0 | 0 | 0 | 48.181 |
| BRTHA2004371 | 0 | 0 | 0 | 0 | 0 | 0 | 100 |
| BRTHA2004582 | 0 | 0 | 0 | 0 | 0 | 0 | 100 |
| BRTHA2004629 | 0 | 0 | 0 | 0 | 0 | 0 | 100 |
| BRTHA2004639 | 0 | 0 | 0 | 0 | 0 | 0 | 100 |
| BRTHA2004642 | 0 | 0 | 0 | 0 | 0 | 0 | 100 |
| BRTHA2004812 | 0 | 0 | 0 | 0 | 0 | 0 | 100 |
| BRTHA2004821 | 0 | 0 | 0 | 0 | 0 | 0 | 100 |
| BRTHA2004978 | 0 | 0 | 0 | 0 | 0 | 0 | 100 |
| BRTHA2004992 | 0 | 0 | 0 | 0 | 0 | 0 | 100 |
| BRTHA2005005 | 0 | 0 | 0 | 0 | 0 | 0 | 100 |
| BRTHA2005013 | 0 | 0 | 0 | 0 | 0 | 0 | 12.374 |
| BRTHA2005448 | 0 | 0 | 0 | 0 | 0 | 0 | 100 |
| BRTHA2005579 | 0 | 0 | 0 | 0 | 0 | 0 | 100 |
| BRTHA2005831 | 0 | 0 | 0 | 0 | 0 | 0 | 48.181 |
| BRTHA2005864 | 1.623 | 0 | 0 | 0 | 0 | 0 | 3.599 |
| BRTHA2005942 | 0 | 0 | 0 | 0 | 0 | 0 | 100 |
| BRTHA2005956 | 0 | 0 | 0 | 0 | 0 | 0 | 100 |
| BRTHA2006075 | 0 | 0 | 0 | 0 | 0 | 0 | 100 |
| BRTHA2006146 | 0 | 0 | 0 | 0 | 0 | 0 | 100 |
| BRTHA2006194 | 0 | 0 | 0 | 0 | 0 | 0 | 100 |
| BRTHA2006232 | 0 | 0 | 0 | 0 | 0 | 0 | 100 |
| BRTHA2006720 | 0 | 0 | 0 | 0 | 0 | 0 | 100 |
| BRTHA2006735 | 0 | 0 | 0 | 0 | 0 | 0 | 100 |
| BRTHA2006974 | 0 | 0 | 0 | 0 | 0 | 0 | 100 |
| BRTHA2006975 | 0 | 0 | 0 | 0 | 0 | 0 | 2.521 |
| BRTHA2007060 | 0 | 0 | 0 | 0 | 0 | 0 | 100 |
| BRTHA2007122 | 0 | 0 | 0 | 0 | 0 | 0 | 100 |
| BRTHA2007422 | 0 | 0 | 0 | 0 | 0 | 0 | 100 |
| BRTHA2007603 | 0 | 0 | 0 | 0 | 0 | 0 | 100 |
| BRTHA2007665 | 0 | 0 | 0 | 0 | 0 | 0 | 100 |
| BRTHA2007998 | 0 | 1.978 | 0 | 0.880 | 3.210 | 6.219 | 1.913 |
| BRTHA2008316 | 0 | 0 | 0 | 0 | 0 | 0 | 100 |
| BRTHA2008335 | 16.281 | 0 | 0 | 8.305 | 0 | 0 | 9.028 |
| BRTHA2008502 | 0 | 0 | 0 | 22.073 | 0 | 0 | 23.993 |
| BRTHA2008513 | 0 | 0 | 0 | 47.917 | 0 | 0 | 52.083 |
| BRTHA2008527 | 0 | 0 | 0 | 0 | 0 | 0 | 100 |
| BRTHA2008535 | 0 | 0 | 0 | 0 | 0 | 0 | 100 |
| BRTHA2008598 | 0 | 0 | 0 | 0 | 0 | 0 | 100 |
| BRTHA2008669 | 0 | 0 | 0 | 0 | 0 | 0 | 100 |
| BRTHA2008955 | 0 | 0 | 0 | 0 | 0 | 0 | 60.975 |
| BRTHA2009311 | 0 | 0 | 0 | 0 | 0 | 0 | 100 |
| BRTHA2009349 | 0 | 0 | 0 | 0 | 0 | 0 | 100 |
| BRTHA2009486 | 0 | 0 | 0 | 11.152 | 0 | 0 | 12.122 |
| BRTHA2009803 | 0 | 0 | 0 | 0 | 0 | 0 | 100 |
| BRTHA2009846 | 0 | 0 | 0 | 0 | 0 | 0 | 100 |
| BRTHA2009886 | 0 | 0 | 0 | 0 | 0 | 0 | 23.052 |
| BRTHA2009972 | 0 | 0 | 0 | 0 | 0 | 0 | 100 |
| BRTHA2010008 | 0 | 0 | 0 | 0 | 0 | 0 | 100 |
| BRTHA2010033 | 0 | 0 | 0 | 0 | 0 | 0 | 100 |
| BRTHA2010073 | 0 | 0 | 0 | 0 | 0 | 0 | 100 |
| BRTHA2010097 | 0 | 0 | 0 | 0 | 0 | 0 | 4.656 |
| BRTHA2010191 | 0 | 0 | 0 | 0 | 0 | 0 | 100 |
| BRTHA2010397 | 0 | 0 | 0 | 0 | 0 | 0 | 100 |
| BRTHA2010608 | 0 | 0 | 0 | 0 | 0 | 0 | 48.181 |
| BRTHA2010672 | 0 | 0 | 0 | 0 | 0 | 0 | 100 |
| BRTHA2010884 | 0 | 0 | 0 | 0 | 0 | 0 | 100 |
| BRTHA2010907 | 0 | 0 | 0 | 0 | 0 | 0 | 100 |
| BRTHA2011062 | 0 | 0 | 0 | 0 | 0 | 0 | 100 |
| BRTHA2011194 | 0 | 0 | 0 | 0 | 0 | 0 | 60.975 |
| BRTHA2011321 | 0 | 0 | 0 | 0 | 0 | 0 | 100 |
| BRTHA2011351 | 0 | 0 | 0 | 0 | 0 | 0 | 100 |
| BRTHA2011500 | 0 | 0 | 0 | 0 | 0 | 0 | 100 |
| BRTHA2011641 | 0 | 0 | 0 | 0 | 0 | 0 | 52.395 |
| BRTHA2011691 | 0 | 0 | 0 | 0 | 0 | 0 | 57.080 |
| BRTHA2012129 | 0 | 0 | 0 | 0 | 0 | 0 | 100 |
| BRTHA2012183 | 0 | 0 | 0 | 0 | 0 | 0 | 48.181 |
| BRTHA2012189 | 0 | 0 | 0 | 0 | 0 | 0 | 100 |
| BRTHA2012351 | 0 | 0 | 0 | 0 | 0 | 0 | 100 |
| BRTHA2012392 | 0 | 0 | 0 | 0 | 0 | 0 | 38.390 |
| BRTHA2012466 | 0 | 0 | 0 | 0 | 0 | 0 | 100 |
| BRTHA2012555 | 0 | 0 | 0 | 0 | 0 | 0 | 100 |
| BRTHA2012562 | 0 | 0 | 0 | 0 | 0 | 0 | 100 |
| BRTHA2012714 | 1.253 | 0 | 0 | 0 | 0 | 0 | 11.120 |
| BRTHA2012980 | 0 | 0 | 0 | 0 | 0 | 0 | 13.890 |
| BRTHA2013054 | 24.028 | 0 | 0 | 0 | 0 | 0 | 13.323 |
| BRTHA2013262 | 0 | 0 | 0 | 0 | 0 | 0 | 100 |
| BRTHA2013275 | 0 | 0 | 0 | 0 | 0 | 0 | 49.749 |
| BRTHA2013426 | 0 | 0 | 0 | 0 | 0 | 0 | 100 |
| BRTHA2013460 | 0 | 0 | 0 | 0 | 0 | 0 | 100 |
| BRTHA2013515 | 47.417 | 0 | 0 | 0 | 0 | 0 | 52.583 |
| BRTHA2013540 | 0 | 0 | 0 | 0 | 0 | 0 | 37.587 |
| BRTHA2013595 | 0 | 0 | 0 | 0 | 0 | 0 | 100 |
| BRTHA2013610 | 0 | 0 | 0 | 0 | 0 | 0 | 100 |
| BRTHA2013707 | 0 | 0 | 0 | 0 | 0 | 0 | 100 |
| BRTHA2013942 | 0 | 0 | 0 | 0 | 0 | 0 | 100 |
| BRTHA2014287 | 0 | 0 | 0 | 0 | 0 | 0 | 100 |
| BRTHA2014334 | 0 | 0 | 0 | 0 | 0 | 0 | 100 |
| BRTHA2014381 | 0 | 0 | 0 | 0 | 0 | 0 | 100 |
| BRTHA2014578 | 0 | 0 | 0 | 0 | 0 | 0 | 100 |
| BRTHA2014647 | 0 | 0 | 0 | 0 | 0 | 0 | 100 |
| BRTHA2014663 | 0 | 0 | 0 | 0 | 0 | 0 | 19.647 |
| BRTHA2014792 | 0 | 0 | 0 | 0 | 0 | 0 | 100 |
| BRTHA2014828 | 0 | 0 | 0 | 0 | 0 | 0 | 100 |
| BRTHA2014909 | 0 | 0 | 0 | 0 | 0 | 0 | 100 |
| BRTHA2014975 | 0 | 0 | 0 | 0 | 0 | 0 | 100 |
| BRTHA2014997 | 0 | 0 | 0 | 0 | 0 | 0 | 57.080 |
| BRTHA2015406 | 0 | 0 | 0 | 0 | 0 | 0 | 100 |
| BRTHA2015478 | 0 | 0 | 0 | 0 | 0 | 0 | 100 |
| BRTHA2015696 | 0 | 0 | 0 | 0 | 0 | 0 | 100 |
| BRTHA2015700 | 0 | 0 | 0 | 0 | 0 | 0 | 100 |
| BRTHA2015853 | 0 | 0 | 0 | 0 | 0 | 0 | 100 |
| BRTHA2015878 | 0 | 0 | 0 | 0 | 0 | 0 | 100 |
| BRTHA2016179 | 0 | 0 | 0 | 0 | 0 | 0 | 100 |
| BRTHA2016215 | 0 | 0 | 0 | 0 | 0 | 0 | 38.930 |
| BRTHA2016267 | 0 | 0 | 0 | 0 | 0 | 0 | 100 |
| BRTHA2016318 | 0 | 0 | 0 | 0 | 0 | 0 | 100 |
| BRTHA2016496 | 0 | 0 | 0 | 0 | 0 | 0 | 100 |
| BRTHA2016543 | 0 | 0 | 0 | 0 | 0 | 0 | 100 |
| BRTHA2016552 | 0 | 0 | 0 | 0 | 0 | 0 | 100 |
| BRTHA2016577 | 0 | 0 | 0 | 0 | 0 | 0 | 2.617 |
| BRTHA2016765 | 0 | 0 | 0 | 0 | 0 | 0 | 13.048 |
| BRTHA2016918 | 0 | 0 | 0 | 0 | 0 | 0 | 32.610 |
| BRTHA2017178 | 0 | 0 | 0 | 0 | 0 | 0 | 48.181 |
| BRTHA2017353 | 0 | 0 | 0 | 0 | 0 | 0 | 100 |
| BRTHA2017364 | 0 | 0 | 0 | 0 | 0 | 0 | 100 |
| BRTHA2017972 | 0 | 0 | 0 | 0 | 0 | 0 | 37.427 |
| BRTHA2017985 | 0 | 0 | 0 | 0 | 0 | 0 | 49.749 |
| BRTHA2018011 | 0 | 0 | 0 | 0 | 0 | 0 | 100 |
| BRTHA2018129 | 0 | 0 | 0 | 0 | 0 | 0 | 100 |
| BRTHA2018165 | 0 | 0 | 0 | 0 | 0 | 0 | 100 |
| BRTHA2018304 | 0 | 0 | 0 | 0 | 0 | 0 | 100 |
| BRTHA2018344 | 0 | 0 | 0 | 0 | 0 | 0 | 100 |
| BRTHA2018420 | 0 | 0 | 0 | 0 | 0 | 0 | 100 |
| BRTHA2018443 | 22.098 | 0 | 0 | 0 | 0 | 0 | 49.012 |
| BRTHA2018504 | 33.981 | 0 | 0 | 0 | 0 | 0 | 37.684 |
| BRTHA2018558 | 0 | 0 | 0 | 0 | 0 | 0 | 100 |
| BRTHA2018591 | 0 | 0 | 0 | 0 | 0 | 0 | 100 |
| BRTHA2018624 | 0 | 0 | 0 | 0 | 0 | 0 | 100 |
| BRTHA2018706 | 0 | 0 | 0 | 0 | 0 | 0 | 100 |
| BRTHA2018707 | 0 | 0 | 0 | 0 | 0 | 0 | 57.080 |
| BRTHA2019014 | 0 | 0 | 0 | 0 | 0 | 0 | 100 |
| BRTHA2019022 | 0 | 0 | 0 | 0 | 0 | 0 | 100 |
| BRTHA2019048 | 0 | 0 | 0 | 0 | 0 | 0 | 100 |
| BRTHA2019073 | 0 | 0 | 0 | 0 | 0 | 0 | 57.080 |
| BRTHA2019726 | 0 | 0 | 0 | 0 | 0 | 0 | 100 |
| BRTHA2019743 | 0 | 0 | 0 | 0 | 0 | 0 | 100 |
| BRTHA2020400 | 0 | 0 | 0 | 0 | 0 | 0 | 100 |
| BRTHA2020566 | 0 | 0 | 0 | 0 | 0 | 0 | 100 |
| BRTHA2020642 | 0 | 0 | 0 | 0 | 0 | 0 | 100 |
| BRTHA2020695 | 0 | 0 | 0 | 0 | 0 | 0 | 100 |
| BRTHA2020721 | 0 | 0 | 0 | 0 | 0 | 0 | 100 |
| BRTHA2020781 | 0 | 0 | 0 | 0 | 0 | 0 | 100 |
| BRTHA2020811 | 0 | 0 | 0 | 0 | 0 | 0 | 100 |
| BRTHA2020910 | 0 | 0 | 0 | 0 | 0 | 0 | 100 |
| BRTHA2021212 | 0 | 0 | 0 | 0 | 0 | 0 | 100 |
| BRTHA2021440 | 0 | 0 | 0 | 0 | 0 | 0 | 57.080 |
| BRTHA2021450 | 0 | 0 | 0 | 0 | 0 | 0 | 100 |
| BRTHA2022074 | 0 | 0 | 0 | 0 | 0 | 0 | 100 |
| BRTHA2022914 | 0 | 0 | 0 | 0 | 0 | 0 | 100 |
| BRTHA2022968 | 0 | 0 | 0 | 0 | 0 | 0 | 100 |
| BRTHA2023402 | 0 | 0 | 0 | 0 | 0 | 0 | 100 |
| BRTHA2023437 | 0 | 0 | 0 | 0 | 0 | 0 | 100 |
| BRTHA2024177 | 0 | 0 | 0 | 0 | 0 | 0 | 100 |
| BRTHA2024354 | 0 | 0 | 0 | 0 | 0 | 0 | 100 |
| BRTHA2024712 | 0 | 0 | 0 | 0 | 0 | 0 | 100 |
| BRTHA2025869 | 0 | 0 | 0 | 0 | 0 | 0 | 100 |
| BRTHA2026071 | 0 | 0 | 0 | 0 | 0 | 0 | 13.612 |
| BRTHA2026290 | 0 | 0 | 0 | 0 | 0 | 0 | 100 |
| BRTHA2026311 | 0 | 0 | 0 | 0 | 0 | 0 | 100 |
| BRTHA2027227 | 0 | 0 | 0 | 0 | 0 | 0 | 100 |
| BRTHA2027229 | 0 | 0 | 0 | 0 | 0 | 0 | 2.030 |
| BRTHA2027250 | 0 | 0 | 0 | 0 | 0 | 0 | 100 |
| BRTHA2027546 | 0 | 0 | 0 | 0 | 0 | 0 | 100 |
| BRTHA2028297 | 0 | 0 | 0 | 0 | 0 | 0 | 100 |
| BRTHA2029969 | 0 | 0 | 0 | 0 | 0 | 0 | 100 |
| BRTHA2030036 | 0 | 0 | 0 | 0 | 0 | 0 | 100 |
| BRTHA2030213 | 0 | 0 | 0 | 0 | 0 | 0 | 100 |
| BRTHA2031517 | 0 | 0 | 0 | 47.917 | 0 | 0 | 52.083 |
| BRTHA2031800 | 0 | 0 | 0 | 0 | 0 | 0 | 100 |
| BRTHA2031917 | 0 | 0 | 0 | 0 | 0 | 0 | 100 |
| BRTHA2032763 | 0 | 0 | 0 | 0 | 0 | 0 | 100 |
| BRTHA2032845 | 0 | 0 | 0 | 0 | 0 | 0 | 100 |
| BRTHA2033122 | 0 | 0 | 0 | 0 | 0 | 0 | 100 |
| BRTHA2033155 | 4.253 | 9.757 | 0 | 0 | 15.830 | 0 | 9.433 |
| BRTHA2033264 | 0 | 67.414 | 0 | 0 | 0 | 0 | 32.586 |
| BRTHA2033320 | 0 | 0 | 0 | 0 | 0 | 0 | 100 |
| BRTHA2033469 | 0 | 0 | 0 | 0 | 0 | 0 | 38.930 |
| BRTHA2033683 | 0 | 0 | 0 | 0 | 0 | 0 | 100 |
| BRTHA2034281 | 0 | 0 | 0 | 0 | 0 | 0 | 100 |
| BRTHA2034576 | 0 | 0 | 0 | 0 | 0 | 0 | 100 |
| BRTHA2034874 | 0 | 0 | 0 | 0 | 0 | 0 | 100 |
| BRTHA2035448 | 0 | 0 | 0 | 0 | 0 | 0 | 100 |
| BRTHA2035743 | 0 | 0 | 0 | 34.617 | 0 | 0 | 22.576 |
| BRTHA2036055 | 0 | 0 | 0 | 0 | 0 | 0 | 100 |
| BRTHA2036295 | 0 | 0 | 0 | 0 | 0 | 0 | 100 |
| BRTHA2036660 | 0 | 0 | 0 | 0 | 0 | 0 | 100 |
| BRTHA2037247 | 0 | 0 | 0 | 0 | 0 | 0 | 100 |
| BRTHA2038279 | 0 | 0 | 0 | 0 | 0 | 0 | 100 |
| BRTHA2038345 | 0 | 0 | 0 | 0 | 0 | 0 | 100 |
| BRTHA2038353 | 0 | 0 | 0 | 0 | 0 | 0 | 100 |
| BRTHA2038396 | 0 | 0 | 0 | 0 | 0 | 0 | 100 |
| BRTHA3000252 | 0 | 0 | 0 | 0 | 0 | 0 | 100 |
| BRTHA3000273 | 0 | 0 | 0 | 0 | 0 | 0 | 57.080 |
| BRTHA3000296 | 0 | 0 | 0 | 0 | 0 | 0 | 100 |
| BRTHA3000297 | 0 | 0 | 0 | 47.917 | 0 | 0 | 52.083 |
| BRTHA3000325 | 0 | 0 | 0 | 0 | 0 | 0 | 100 |
| BRTHA3000456 | 0 | 0 | 0 | 0 | 0 | 0 | 62.915 |
| BRTHA3000633 | 0 | 0 | 0 | 0 | 0 | 0 | 37.898 |
| BRTHA3000789 | 0 | 0 | 0 | 35.937 | 0 | 0 | 39.062 |
| BRTHA3001221 | 0 | 0 | 0 | 0 | 0 | 0 | 100 |
| BRTHA3001623 | 0 | 0 | 0 | 0 | 0 | 0 | 100 |
| BRTHA3002265 | 0 | 0 | 0 | 0 | 0 | 0 | 100 |
| BRTHA3002401 | 0 | 0 | 0 | 0 | 0 | 0 | 100 |
| BRTHA3002411 | 0 | 0 | 0 | 0 | 0 | 0 | 100 |
| BRTHA3002427 | 8.577 | 0 | 10.128 | 2.917 | 10.641 | 10.308 | 15.852 |
| BRTHA3002864 | 0 | 0 | 0 | 0 | 0 | 0 | 57.080 |
| BRTHA3002933 | 0 | 0 | 0 | 0 | 0 | 0 | 100 |
| BRTHA3002955 | 0 | 0 | 0 | 0 | 0 | 0 | 100 |
| BRTHA3003000 | 17.150 | 0 | 0 | 0 | 63.832 | 0 | 19.019 |
| BRTHA3003023 | 0 | 4.637 | 7.160 | 2.062 | 0 | 7.287 | 4.483 |
| BRTHA3003074 | 0 | 0 | 0 | 0 | 0 | 0 | 37.459 |
| BRTHA3003225 | 0 | 0 | 0 | 0 | 0 | 0 | 47.165 |
| BRTHA3003339 | 0 | 0 | 0 | 0 | 0 | 0 | 100 |
| BRTHA3003343 | 0 | 0 | 0 | 0 | 0 | 0 | 100 |
| BRTHA3003417 | 0 | 0 | 0 | 31.398 | 0 | 0 | 34.129 |
| BRTHA3003449 | 0 | 0 | 0 | 0 | 0 | 0 | 100 |
| BRTHA3003474 | 0 | 0 | 0 | 0 | 0 | 0 | 100 |
| BRTHA3003490 | 1.623 | 3.724 | 0 | 0 | 0 | 0 | 1.800 |
| BRTHA3003704 | 0 | 0 | 0 | 0 | 0 | 0 | 60.975 |
| BRTHA3003736 | 4.302 | 0 | 0 | 6.584 | 0 | 0 | 2.385 |
| BRTHA3004307 | 9.037 | 0 | 0 | 24.587 | 0 | 0 | 6.681 |
| BRTHA3004432 | 32.087 | 0 | 0 | 0 | 0 | 0 | 35.583 |
| BRTHA3004475 | 0 | 0 | 0 | 0 | 0 | 0 | 52.395 |
| BRTHA3004502 | 0 | 0 | 0 | 0 | 0 | 0 | 100 |
| BRTHA3004610 | 0 | 0 | 0 | 0 | 0 | 0 | 100 |
| BRTHA3005046 | 0 | 0 | 0 | 47.917 | 0 | 0 | 52.083 |
| BRTHA3005735 | 0 | 0 | 0 | 0 | 0 | 0 | 100 |
| BRTHA3005988 | 31.076 | 0 | 0 | 0 | 0 | 0 | 68.924 |
| BRTHA3006318 | 0 | 0 | 0 | 0 | 0 | 0 | 100 |
| BRTHA3006593 | 0 | 0 | 0 | 0 | 0 | 0 | 100 |
| BRTHA3006856 | 0 | 0 | 0 | 0 | 0 | 0 | 100 |
| BRTHA3007113 | 0 | 0 | 0 | 0 | 0 | 0 | 100 |
| BRTHA3007130 | 0 | 0 | 0 | 0 | 0 | 0 | 100 |
| BRTHA3007148 | 0 | 0 | 0 | 0 | 0 | 0 | 100 |
| BRTHA3007319 | 0 | 0 | 0 | 0 | 0 | 0 | 100 |
| BRTHA3007469 | 0 | 0 | 0 | 0 | 0 | 0 | 100 |
| BRTHA3007472 | 0 | 0 | 0 | 7.962 | 0 | 0 | 4.327 |
| BRTHA3007532 | 0 | 0 | 0 | 0 | 0 | 0 | 100 |
| BRTHA3007662 | 0 | 0 | 0 | 64.789 | 0 | 0 | 35.211 |
| BRTHA3007685 | 0 | 0 | 0 | 0 | 0 | 0 | 60.975 |
| BRTHA3007769 | 0 | 0 | 0 | 0 | 0 | 0 | 100 |
| BRTHA3007922 | 0 | 0 | 0 | 0 | 0 | 0 | 34.613 |
| BRTHA3008143 | 0 | 0 | 0 | 0 | 0 | 0 | 100 |
| BRTHA3008293 | 0 | 0 | 0 | 0 | 0 | 0 | 100 |
| BRTHA3008310 | 0 | 0 | 0 | 0 | 0 | 0 | 100 |
| BRTHA3008386 | 0 | 0 | 0 | 0 | 0 | 0 | 100 |
| BRTHA3008520 | 47.417 | 0 | 0 | 0 | 0 | 0 | 52.583 |
| BRTHA3008608 | 0 | 0 | 0 | 0 | 0 | 0 | 100 |
| BRTHA3008694 | 0 | 0 | 0 | 0 | 0 | 0 | 100 |
| BRTHA3008778 | 0 | 0 | 0 | 25.346 | 0 | 0 | 27.551 |
| BRTHA3008826 | 0 | 0 | 0 | 0 | 0 | 0 | 100 |
| BRTHA3009037 | 0 | 0 | 0 | 0 | 0 | 0 | 100 |
| BRTHA3009090 | 0 | 0 | 0 | 12.008 | 0 | 0 | 13.052 |
| BRTHA3009291 | 0 | 0 | 0 | 0 | 0 | 0 | 100 |
| BRTHA3009858 | 0 | 0 | 0 | 0 | 0 | 0 | 25.720 |
| BRTHA3010135 | 20.010 | 0 | 0 | 0 | 0 | 0 | 44.380 |
| BRTHA3010366 | 0 | 0 | 0 | 0 | 0 | 0 | 100 |
| BRTHA3010469 | 0 | 0 | 0 | 0 | 0 | 0 | 100 |
| BRTHA3010530 | 0 | 0 | 0 | 0 | 0 | 0 | 31.009 |
| BRTHA3010540 | 0 | 0 | 0 | 0 | 0 | 0 | 7.466 |
| BRTHA3010717 | 0 | 0 | 0 | 0 | 0 | 0 | 100 |
| BRTHA3011149 | 0 | 0 | 0 | 0 | 0 | 0 | 100 |
| BRTHA3011187 | 0 | 0 | 0 | 0 | 0 | 0 | 100 |
| BRTHA3011194 | 3.534 | 0 | 0 | 10.818 | 0 | 0 | 3.920 |
| BRTHA3011229 | 0 | 0 | 0 | 0 | 0 | 0 | 100 |
| BRTHA3011265 | 25.596 | 0 | 0 | 0 | 0 | 0 | 18.923 |
| BRTHA3011361 | 0 | 0 | 0 | 0 | 0 | 0 | 62.915 |
| BRTHA3011510 | 0 | 0 | 0 | 0 | 0 | 0 | 100 |
| BRTHA3011892 | 0 | 0 | 0 | 0 | 0 | 0 | 100 |
| BRTHA3012265 | 0 | 0 | 0 | 64.789 | 0 | 0 | 35.211 |
| BRTHA3013860 | 0 | 0 | 0 | 0 | 0 | 0 | 100 |
| BRTHA3013882 | 0 | 0 | 0 | 0 | 0 | 0 | 100 |
| BRTHA3013884 | 0 | 0 | 0 | 0 | 0 | 0 | 100 |
| BRTHA3013981 | 0 | 0 | 0 | 0 | 0 | 0 | 100 |
| BRTHA3014000 | 0 | 0 | 0 | 0 | 0 | 0 | 100 |
| BRTHA3014105 | 0 | 0 | 0 | 0 | 0 | 0 | 100 |
| BRTHA3014507 | 0 | 0 | 0 | 0 | 0 | 0 | 100 |
| BRTHA3014547 | 0 | 0 | 0 | 0 | 0 | 0 | 100 |
| BRTHA3014691 | 0 | 0 | 0 | 0 | 0 | 0 | 100 |
| BRTHA3014835 | 0 | 0 | 0 | 0 | 0 | 0 | 100 |
| BRTHA3014854 | 0 | 0 | 0 | 0 | 0 | 0 | 100 |
| BRTHA3014908 | 0 | 0 | 0 | 0 | 0 | 0 | 100 |
| BRTHA3014920 | 0 | 0 | 0 | 0 | 0 | 0 | 100 |
| BRTHA3015815 | 0 | 0 | 0 | 0 | 0 | 0 | 100 |
| BRTHA3016616 | 0 | 0 | 0 | 0 | 0 | 0 | 100 |
| BRTHA3016835 | 0 | 0 | 0 | 0 | 0 | 0 | 100 |
| BRTHA3016845 | 0 | 0 | 0 | 0 | 0 | 0 | 100 |
| BRTHA3016917 | 0 | 0 | 0 | 0 | 0 | 0 | 100 |
| BRTHA3017047 | 0 | 0 | 0 | 0 | 0 | 0 | 100 |
| BRTHA3017589 | 0 | 0 | 0 | 0 | 0 | 0 | 100 |
| BRTHA3017848 | 47.417 | 0 | 0 | 0 | 0 | 0 | 52.583 |
| BRTHA3018409 | 0 | 0 | 0 | 0 | 0 | 0 | 100 |
| BRTHA3018514 | 0 | 0 | 0 | 0 | 0 | 0 | 100 |
| BRTHA3018617 | 0 | 0 | 0 | 0 | 0 | 0 | 100 |
| BRTHA3018623 | 0 | 0 | 0 | 0 | 0 | 0 | 100 |
| BRTHA3018656 | 0 | 0 | 0 | 0 | 0 | 0 | 100 |
| BRTHA3019048 | 0 | 0 | 0 | 0 | 0 | 0 | 100 |
| BRTHA3019105 | 0 | 0 | 0 | 0 | 0 | 0 | 100 |
| BRTHA3019183 | 0 | 0 | 0 | 0 | 0 | 0 | 100 |
| BRTHA3020000 | 0 | 0 | 0 | 0 | 0 | 0 | 100 |
| BRTHA3020314 | 10.182 | 0 | 0 | 0 | 0 | 0 | 22.584 |
| BRTHA3020369 | 0 | 0 | 0 | 0 | 0 | 0 | 100 |
| BRTHA3021569 | 0 | 0 | 0 | 0 | 0 | 0 | 100 |
| BRTHA3021708 | 0 | 0 | 0 | 0 | 0 | 0 | 60.975 |
| BRTHA3021786 | 0 | 0 | 0 | 0 | 0 | 0 | 100 |
| BRTHA3021971 | 1.941 | 0 | 6.876 | 1.980 | 0 | 0 | 4.305 |
| BRTHA3022641 | 0 | 0 | 0 | 0 | 0 | 0 | 19.618 |
| BRTHA3023403 | 14.857 | 0 | 0 | 15.157 | 0 | 0 | 8.238 |
| BRTHA3023523 | 0 | 0 | 0 | 0 | 0 | 0 | 100 |
| BRTHA3023590 | 0 | 0 | 0 | 0 | 0 | 0 | 100 |
| BRTHA3023929 | 0 | 0 | 0 | 0 | 0 | 0 | 100 |
| BRTHA3024233 | 0 | 0 | 0 | 0 | 0 | 0 | 100 |
| BRTHA3024600 | 0 | 0 | 0 | 0 | 0 | 0 | 100 |
| BRTHA3025073 | 0 | 0 | 0 | 0 | 0 | 0 | 100 |
| BRTHA3025789 | 0 | 0 | 0 | 0 | 0 | 0 | 100 |
| BRTHA3025826 | 0 | 0 | 0 | 0 | 0 | 0 | 100 |
| BRTHA3026161 | 0 | 0 | 0 | 0 | 0 | 0 | 100 |
| BRTHA3026180 | 0 | 0 | 0 | 0 | 0 | 0 | 100 |
| BRTHA3026507 | 10.482 | 0 | 12.377 | 7.129 | 13.004 | 0 | 38.746 |
| BRTHA3026556 | 0 | 0 | 0 | 0 | 0 | 0 | 100 |
| BRTHA3026916 | 0 | 0 | 0 | 0 | 0 | 0 | 52.395 |
| BRTHA3027171 | 0 | 0 | 0 | 0 | 0 | 0 | 100 |
| BRTHA3027318 | 0 | 0 | 0 | 0 | 0 | 0 | 100 |
| BRTHA3027638 | 0 | 0 | 0 | 0 | 0 | 0 | 100 |
| BRTHA3027820 | 0 | 0 | 0 | 0 | 0 | 0 | 100 |
| BRTHA3027879 | 0 | 0 | 0 | 0 | 0 | 0 | 48.181 |
| BRTHA3027957 | 0 | 0 | 0 | 0 | 0 | 0 | 100 |
| BRTHA3028339 | 0 | 0 | 0 | 0 | 0 | 0 | 100 |
| BRTHA3028505 | 0 | 0 | 0 | 0 | 0 | 0 | 100 |
| BRTHA3028782 | 0 | 0 | 0 | 0 | 0 | 0 | 100 |
| CD34C3000125 | 0 | 0 | 0 | 2.372 | 0 | 0 | 0 |
| CD34C3000252 | 0 | 0 | 0 | 0.837 | 0 | 0 | 0.910 |
| CD34C3000424 | 0 | 0 | 0 | 2.336 | 0 | 0 | 0 |
| CHONS2000172 | 3.736 | 0 | 0 | 0 | 0 | 0 | 0 |
| CHONS2002829 | 0 | 0 | 0 | 11.668 | 0 | 0 | 1.812 |
| COLON2001721 | 11.065 | 0 | 0 | 5.644 | 0 | 0 | 0 |
| COLON2007733 | 12.468 | 0 | 0 | 0 | 0 | 0 | 0 |
| CTONG1000010 | 0 | 0 | 0 | 0 | 0 | 0 | 7.190 |
| CTONG1000052 | 4.834 | 0 | 0 | 1.849 | 2.249 | 4.357 | 1.340 |
| CTONG1000087 | 0 | 2.399 | 0 | 1.067 | 0 | 0 | 3.479 |
| CTONG1000088 | 0 | 0 | 0 | 2.981 | 0 | 0 | 0 |
| CTONG1000094 | 0 | 1.787 | 4.139 | 2.781 | 1.450 | 0 | 0.864 |
| CTONG1000180 | 0 | 0 | 0 | 0 | 0 | 0 | 26.631 |
| CTONG1000218 | 0 | 1.105 | 0 | 0.491 | 1.792 | 0 | 0.534 |
| CTONG1000241 | 4.243 | 0 | 0 | 8.657 | 0 | 0 | 4.705 |
| CTONG1000467 | 0 | 0 | 30.440 | 8.766 | 0 | 0 | 0 |
| CTONG2000031 | 34.934 | 0 | 0 | 0 | 0 | 0 | 0 |
| CTONG2000042 | 0 | 0 | 0 | 3.574 | 0 | 0 | 0 |
| CTONG2000063 | 2.141 | 0 | 0 | 0 | 0 | 0 | 4.748 |
| CTONG2000411 | 5.755 | 0 | 0 | 0 | 0 | 0 | 6.382 |
| CTONG2000452 | 0 | 0 | 0 | 2.971 | 0 | 0 | 0 |
| CTONG2000469 | 0 | 0 | 10.345 | 0 | 0 | 0 | 0 |
| CTONG2000508 | 7.107 | 1.812 | 2.798 | 1.611 | 0 | 0 | 1.751 |
| CTONG2000827 | 0 | 0 | 0 | 0 | 0 | 0 | 37.320 |
| CTONG2001226 | 0 | 0 | 0 | 7.744 | 0 | 0 | 0 |
| CTONG2001320 | 0 | 0 | 0 | 0 | 19.622 | 0 | 0 |
| CTONG2001533 | 0 | 0 | 0 | 35.391 | 0 | 0 | 0 |
| CTONG2001748 | 2.090 | 0 | 0 | 0 | 0 | 0 | 0 |
| CTONG2001800 | 0 | 0 | 0 | 28.849 | 0 | 0 | 0 |
| CTONG2001911 | 0 | 0 | 0 | 15.416 | 0 | 0 | 0 |
| CTONG2002073 | 0 | 6.721 | 5.189 | 0 | 0 | 0 | 0 |
| CTONG2002095 | 7.003 | 0 | 0 | 7.145 | 0 | 0 | 0 |
| CTONG2002270 | 14.322 | 0 | 0 | 14.611 | 0 | 0 | 8.235 |
| CTONG2002744 | 4.711 | 2.702 | 0 | 2.403 | 4.384 | 6.370 | 5.225 |
| CTONG2002766 | 0 | 0 | 0 | 12.054 | 0 | 0 | 0 |
| CTONG2002803 | 10.307 | 3.378 | 0 | 15.022 | 0 | 0 | 3.266 |
| CTONG2002832 | 0 | 0 | 0 | 0 | 0 | 0 | 12.432 |
| CTONG2002841 | 21.185 | 0 | 0 | 10.807 | 0 | 0 | 0 |
| CTONG2003094 | 5.497 | 0 | 6.491 | 5.608 | 0 | 6.606 | 10.159 |
| CTONG2003298 | 1.451 | 1.665 | 2.571 | 2.221 | 0 | 0 | 0.805 |
| CTONG2003524 | 6.445 | 0 | 0 | 1.644 | 0 | 0 | 0 |
| CTONG2004305 | 34.934 | 0 | 0 | 0 | 0 | 0 | 0 |
| CTONG2004423 | 22.982 | 0 | 0 | 0 | 0 | 0 | 0 |
| CTONG2004487 | 10.405 | 0 | 3.351 | 14.475 | 0 | 0 | 3.147 |
| CTONG2005028 | 6.845 | 0 | 0 | 0 | 0 | 0 | 7.591 |
| CTONG2005145 | 0 | 0 | 0 | 25.034 | 0 | 0 | 0 |
| CTONG2005278 | 0 | 0 | 0 | 2.940 | 0 | 0 | 0 |
| CTONG2005553 | 1.432 | 0 | 0 | 3.653 | 0 | 0 | 1.588 |
| CTONG2005795 | 0 | 0 | 0 | 35.391 | 0 | 0 | 0 |
| CTONG2006004 | 1.055 | 0 | 0 | 0 | 0 | 0 | 1.170 |
| CTONG2006235 | 9.828 | 0 | 11.606 | 5.014 | 6.097 | 0 | 5.450 |
| CTONG2006377 | 11.740 | 0 | 0 | 0 | 0 | 0 | 0 |
| CTONG2006393 | 34.934 | 0 | 0 | 0 | 0 | 0 | 0 |
| CTONG2006412 | 6.207 | 0 | 0 | 6.333 | 23.104 | 0 | 6.884 |
| CTONG2006449 | 0 | 0 | 31.141 | 8.969 | 0 | 0 | 0 |
| CTONG2006562 | 4.200 | 0 | 0 | 1.428 | 0 | 0 | 1.553 |
| CTONG2006709 | 15.516 | 0 | 0 | 7.915 | 9.625 | 0 | 0 |
| CTONG2007072 | 0 | 4.936 | 0 | 0 | 8.007 | 0 | 0 |
| CTONG2007078 | 2.556 | 0 | 0 | 2.608 | 0 | 0 | 1.417 |
| CTONG2007104 | 0 | 14.269 | 0 | 0 | 46.299 | 0 | 0 |
| CTONG2007168 | 24.181 | 0 | 0 | 24.670 | 0 | 0 | 6.704 |
| CTONG2007293 | 0 | 0 | 0 | 0 | 0 | 0 | 37.320 |
| CTONG2007399 | 16.311 | 0 | 0 | 0 | 0 | 0 | 0 |
| CTONG2007400 | 0 | 0 | 0 | 11.740 | 0 | 0 | 0 |
| CTONG2007417 | 0 | 0 | 0 | 0 | 0 | 0 | 23.904 |
| CTONG2007500 | 1.504 | 1.725 | 0 | 0.767 | 0 | 2.712 | 2.502 |
| CTONG2007586 | 0 | 0 | 21.292 | 0 | 0 | 0 | 0 |
| CTONG2007613 | 0 | 0 | 0 | 0 | 0 | 0 | 9.707 |
| CTONG2007681 | 0 | 0 | 0 | 18.854 | 0 | 0 | 20.493 |
| CTONG2007776 | 8.506 | 0 | 15.067 | 4.339 | 0 | 0 | 9.433 |
| CTONG2007779 | 0 | 0 | 0 | 0 | 0 | 0 | 37.320 |
| CTONG2008014 | 0 | 0 | 0 | 15.447 | 56.353 | 0 | 0 |
| CTONG2008321 | 0 | 0 | 53.293 | 0 | 0 | 0 | 0 |
| CTONG2008398 | 2.294 | 0 | 2.708 | 1.560 | 0 | 0 | 1.696 |
| CTONG2008405 | 0 | 0 | 0 | 22.452 | 0 | 0 | 0 |
| CTONG2008506 | 3.764 | 0 | 0 | 3.840 | 0 | 0 | 2.087 |
| CTONG2008518 | 7.703 | 0 | 0 | 3.929 | 0 | 0 | 4.271 |
| CTONG2008521 | 0 | 0 | 0 | 15.511 | 0 | 0 | 0 |
| CTONG2008689 | 0 | 0 | 0 | 21.321 | 0 | 0 | 0 |
| CTONG2008721 | 0 | 0 | 0 | 0 | 0 | 0 | 37.320 |
| CTONG2009033 | 25.755 | 0 | 0 | 26.276 | 0 | 0 | 0 |
| CTONG2009108 | 0 | 0 | 0 | 0 | 0 | 0 | 26.317 |
| CTONG2009423 | 0 | 0 | 0 | 35.391 | 0 | 0 | 0 |
| CTONG2009529 | 0 | 14.506 | 0 | 0 | 0 | 0 | 0 |
| CTONG2009534 | 0 | 0 | 0 | 23.537 | 28.623 | 0 | 8.528 |
| CTONG2009938 | 0 | 0 | 0 | 0 | 0 | 0 | 22.188 |
| CTONG2010348 | 3.542 | 4.063 | 0 | 1.807 | 6.591 | 0 | 3.927 |
| CTONG2010408 | 17.136 | 0 | 0 | 0 | 0 | 0 | 0 |
| CTONG2010652 | 0 | 0 | 0 | 0 | 0 | 0 | 35.648 |
| CTONG2010821 | 1.429 | 0 | 0 | 2.187 | 0 | 0 | 3.170 |
| CTONG2011188 | 0 | 0 | 0 | 21.500 | 0 | 0 | 0 |
| CTONG2011801 | 0 | 0 | 0 | 0 | 11.021 | 0 | 3.284 |
| CTONG2012001 | 0 | 0 | 0 | 7.295 | 0 | 0 | 0 |
| CTONG2012050 | 7.104 | 0 | 0 | 0 | 0 | 0 | 0 |
| CTONG2012452 | 0.712 | 4.085 | 1.261 | 0 | 0 | 0 | 0.790 |
| CTONG2012607 | 0 | 0 | 0 | 0 | 0 | 0 | 5.754 |
| CTONG2012745 | 0 | 0 | 0 | 5.659 | 0 | 0 | 0 |
| CTONG2012847 | 3.444 | 0 | 0 | 0 | 0 | 0 | 1.910 |
| CTONG2012879 | 0 | 0 | 0 | 0 | 0 | 0 | 1.886 |
| CTONG2012996 | 2.920 | 0 | 0 | 0 | 0 | 0 | 6.476 |
| CTONG2013348 | 5.501 | 0 | 0 | 0 | 0 | 0 | 12.201 |
| CTONG2013381 | 6.274 | 7.197 | 0 | 0 | 11.676 | 0 | 0 |
| CTONG2013907 | 2.156 | 0 | 0 | 0 | 0 | 0 | 2.391 |
| CTONG2013934 | 6.936 | 0 | 0 | 7.077 | 0 | 0 | 7.692 |
| CTONG2014119 | 0 | 0 | 0 | 0 | 0 | 0 | 8.988 |
| CTONG2014165 | 0 | 0 | 0 | 0 | 0 | 0 | 5.339 |
| CTONG2014369 | 0 | 0 | 0 | 4.246 | 0 | 0 | 0 |
| CTONG2014389 | 0 | 0 | 0 | 2.695 | 0 | 0 | 2.929 |
| CTONG2014898 | 5.768 | 13.233 | 0 | 0 | 0 | 0 | 6.396 |
| CTONG2015091 | 0 | 0 | 0 | 0 | 0 | 0 | 1.801 |
| CTONG2015204 | 0 | 0 | 0 | 15.475 | 0 | 0 | 0 |
| CTONG2015330 | 28.439 | 0 | 0 | 0 | 0 | 0 | 0 |
| CTONG2015345 | 0 | 0 | 0 | 0 | 31.774 | 0 | 0 |
| CTONG2015518 | 26.559 | 0 | 0 | 0 | 0 | 0 | 0 |
| CTONG2015815 | 8.797 | 0 | 0 | 0 | 0 | 0 | 0 |
| CTONG2016056 | 3.977 | 0 | 0 | 0 | 0 | 0 | 0 |
| CTONG2016408 | 0 | 0 | 0 | 3.777 | 0 | 0 | 4.106 |
| CTONG2016499 | 34.934 | 0 | 0 | 0 | 0 | 0 | 0 |
| CTONG2016575 | 1.989 | 1.141 | 0 | 3.551 | 0 | 0 | 0.551 |
| CTONG2016869 | 2.641 | 0 | 0 | 0 | 0 | 0 | 0 |
| CTONG2016931 | 3.052 | 7.001 | 0 | 12.454 | 0 | 0 | 3.384 |
| CTONG2017094 | 11.178 | 0 | 0 | 0 | 0 | 0 | 0 |
| CTONG2017409 | 0 | 0 | 0 | 0 | 0 | 0 | 37.320 |
| CTONG2017500 | 2.649 | 0 | 0 | 0 | 0 | 0 | 2.938 |
| CTONG2017998 | 2.505 | 0 | 0 | 5.112 | 9.324 | 0 | 0 |
| CTONG2018211 | 13.642 | 0 | 0 | 13.918 | 0 | 0 | 3.782 |
| CTONG2018383 | 0 | 0 | 0 | 0 | 0 | 0 | 5.013 |
| CTONG2018413 | 25.179 | 0 | 0 | 0 | 0 | 0 | 27.923 |
| CTONG2018637 | 0 | 0 | 0 | 6.693 | 0 | 0 | 0 |
| CTONG2018739 | 3.875 | 0 | 0 | 0 | 0 | 0 | 0 |
| CTONG2018898 | 7.706 | 0 | 1.706 | 6.388 | 0 | 5.210 | 5.341 |
| CTONG2019029 | 8.092 | 0 | 0 | 0 | 0 | 0 | 0 |
| CTONG2019063 | 21.231 | 0 | 0 | 0 | 0 | 0 | 0 |
| CTONG2019590 | 0 | 0 | 0 | 0 | 0 | 0 | 11.933 |
| CTONG2019788 | 0 | 0 | 0 | 3.120 | 0 | 0 | 3.391 |
| CTONG2020374 | 31.081 | 0 | 0 | 0 | 0 | 0 | 0 |
| CTONG2020378 | 16.140 | 0 | 0 | 16.467 | 0 | 0 | 0 |
| CTONG2020445 | 0 | 0 | 0 | 0 | 0 | 0 | 23.341 |
| CTONG2020484 | 0 | 0 | 0 | 0.600 | 0 | 2.120 | 0.652 |
| CTONG2020560 | 18.002 | 0 | 0 | 4.591 | 0 | 0 | 3.327 |
| CTONG2020582 | 9.594 | 0 | 0 | 1.958 | 0 | 0 | 4.256 |
| CTONG2020831 | 0 | 0 | 0 | 0 | 0 | 0 | 37.320 |
| CTONG2023021 | 0 | 0 | 0 | 0 | 0 | 0 | 37.320 |
| CTONG2024031 | 2.584 | 0 | 0 | 0 | 0 | 0 | 0 |
| CTONG2027263 | 16.468 | 0 | 0 | 0 | 0 | 0 | 0 |
| CTONG2028124 | 0.503 | 2.306 | 1.780 | 1.026 | 0 | 0 | 1.672 |
| CTONG2028208 | 0 | 0 | 0 | 0 | 0 | 0 | 3.927 |
| CTONG2028309 | 0 | 0 | 0 | 0 | 0 | 0 | 8.140 |
| CTONG2028486 | 0 | 0 | 0 | 35.391 | 0 | 0 | 0 |
| CTONG3000657 | 3.880 | 8.901 | 0 | 11.875 | 0 | 0 | 8.605 |
| CTONG3001123 | 7.847 | 0 | 0 | 0 | 0 | 0 | 0 |
| CTONG3001746 | 42.917 | 0 | 0 | 0 | 0 | 0 | 0 |
| CTONG3002412 | 0 | 0 | 0 | 10.918 | 0 | 0 | 0 |
| CTONG3002835 | 0 | 2.521 | 0 | 4.483 | 0 | 0 | 0 |
| CTONG3004072 | 0 | 0 | 0 | 19.915 | 0 | 0 | 0 |
| CTONG3004726 | 0 | 0 | 0 | 17.278 | 0 | 0 | 0 |
| CTONG3008894 | 0 | 0 | 0 | 2.966 | 0 | 0 | 2.418 |
| CTONG3009028 | 0 | 0 | 0 | 0 | 0 | 0 | 1.785 |
| CTONG3009239 | 0 | 0 | 5.115 | 2.946 | 0 | 0 | 1.601 |
| CTONG3009328 | 11.993 | 0 | 0 | 0 | 0 | 0 | 0 |
| D3OST1000267 | 0 | 9.907 | 0 | 0 | 0 | 15.570 | 0 |
| D3OST1000270 | 0 | 0 | 9.153 | 0 | 0 | 0 | 0 |
| D6OST2000358 | 0 | 0 | 0 | 0 | 0 | 0 | 1.576 |
| D6OST2000464 | 0 | 0 | 0 | 1.118 | 4.078 | 0 | 0 |
| D9OST2000031 | 0 | 0 | 16.736 | 0 | 0 | 0 | 0 |
| D9OST2002608 | 6.684 | 0 | 0 | 4.546 | 0 | 0 | 2.471 |
| D9OST2003106 | 6.904 | 0 | 0 | 0 | 0 | 0 | 0 |
| D9OST2003762 | 0 | 0 | 0 | 0 | 8.112 | 0 | 0 |
| DFNES1000083 | 2.249 | 0 | 15.938 | 2.295 | 0 | 0 | 0 |
| DFNES1000150 | 0 | 0 | 0 | 1.139 | 0 | 0 | 1.238 |
| DFNES2000011 | 0 | 1.255 | 0 | 0 | 2.035 | 3.943 | 0 |
| DFNES2000153 | 0 | 0 | 0 | 0 | 0 | 0 | 3.551 |
| DFNES2000268 | 0 | 2.533 | 0 | 1.127 | 0 | 0 | 0 |
| DFNES2000292 | 0 | 8.610 | 0 | 0 | 0 | 0 | 0 |
| DFNES2000335 | 0 | 0 | 16.783 | 4.833 | 0 | 0 | 0 |
| DFNES2000443 | 0 | 0 | 2.405 | 0 | 0 | 0 | 0.753 |
| DFNES2000457 | 0 | 0 | 0 | 0 | 0 | 0 | 9.850 |
| DFNES2000526 | 0 | 0 | 0 | 0 | 0 | 0 | 3.065 |
| DFNES2001091 | 0 | 19.689 | 0 | 0 | 0 | 0 | 0 |
| DFNES2001096 | 0 | 0 | 27.148 | 0 | 0 | 0 | 0 |
| DFNES2001829 | 9.228 | 0 | 0 | 18.830 | 0 | 0 | 0 |
| DFNES2004371 | 4.733 | 0 | 0 | 0 | 0 | 0 | 1.750 |
| DFNES2005690 | 5.077 | 0 | 0 | 15.539 | 0 | 0 | 11.260 |
| DFNES2007113 | 0 | 0 | 0 | 0 | 0 | 0 | 7.411 |
| DFNES2007634 | 0 | 6.118 | 6.298 | 0.907 | 3.308 | 0 | 4.928 |
| DFNES2008088 | 0 | 6.031 | 0 | 2.682 | 0 | 0 | 2.915 |
| DFNES2008160 | 4.480 | 0 | 0 | 4.571 | 0 | 16.153 | 9.936 |
| DFNES2009482 | 1.418 | 0 | 0 | 0 | 0 | 0 | 1.573 |
| DFNES2011245 | 0 | 0 | 0 | 14.757 | 0 | 0 | 0 |
| DFNES2011380 | 11.690 | 0 | 0 | 0 | 0 | 0 | 0 |
| DFNES2011499 | 0 | 0 | 0 | 6.330 | 0 | 0 | 0 |
| FCBBF1000024 | 6.587 | 0 | 0 | 6.720 | 0 | 7.916 | 0 |
| FCBBF1000025 | 0 | 7.342 | 0 | 6.530 | 11.911 | 0 | 0 |
| FCBBF1000027 | 0.733 | 2.521 | 2.595 | 1.495 | 2.727 | 3.962 | 1.625 |
| FCBBF1000061 | 7.056 | 5.396 | 0 | 2.399 | 0 | 0 | 7.824 |
| FCBBF1000063 | 11.962 | 0 | 0 | 6.102 | 0 | 0 | 0 |
| FCBBF1000077 | 0 | 0 | 0 | 0 | 0 | 0 | 0.898 |
| FCBBF1000118 | 0 | 0 | 0 | 15.471 | 0 | 0 | 0 |
| FCBBF1000121 | 0 | 0 | 35.196 | 0 | 0 | 0 | 0 |
| FCBBF1000139 | 3.496 | 0 | 0 | 1.784 | 0 | 0 | 0 |
| FCBBF1000197 | 4.273 | 0 | 3.784 | 7.629 | 0 | 0 | 3.554 |
| FCBBF1000272 | 0 | 0 | 0 | 35.577 | 0 | 0 | 0 |
| FCBBF1000294 | 0 | 0 | 0 | 2.197 | 4.007 | 0 | 1.194 |
| FCBBF1000367 | 0 | 0 | 0 | 1.550 | 0 | 0 | 0 |
| FCBBF1000376 | 21.300 | 0 | 0 | 0 | 0 | 0 | 0 |
| FCBBF1000403 | 0 | 0 | 0 | 0 | 31.217 | 0 | 0 |
| FCBBF1000412 | 5.806 | 0 | 0 | 0 | 0 | 0 | 2.146 |
| FCBBF1000449 | 13.371 | 0 | 0 | 0 | 0 | 0 | 7.414 |
| FCBBF1000466 | 1.911 | 0 | 0 | 0.650 | 0 | 0 | 5.650 |
| FCBBF1000476 | 26.054 | 0 | 0 | 8.860 | 0 | 0 | 0 |
| FCBBF1000509 | 3.732 | 17.123 | 19.830 | 5.711 | 6.945 | 6.728 | 6.208 |
| FCBBF1000556 | 9.594 | 9.783 | 15.106 | 9.788 | 3.968 | 7.687 | 16.550 |
| FCBBF1000563 | 0 | 0 | 0 | 1.532 | 11.180 | 0 | 6.662 |
| FCBBF1000574 | 8.303 | 0 | 7.354 | 6.353 | 0 | 0 | 4.604 |
| FCBBF1000671 | 0 | 0 | 0 | 13.968 | 0 | 0 | 0 |
| FCBBF1000684 | 0 | 0 | 0 | 0 | 66.829 | 0 | 0 |
| FCBBF1000748 | 0 | 0 | 0 | 0 | 0 | 0 | 37.510 |
| FCBBF1000760 | 0.655 | 0 | 0 | 0 | 0 | 0 | 1.452 |
| FCBBF2000038 | 0 | 3.082 | 0 | 0 | 0 | 4.843 | 0 |
| FCBBF2000087 | 0 | 0 | 0 | 0 | 0 | 10.402 | 0 |
| FCBBF2000199 | 11.190 | 0 | 0 | 2.854 | 0 | 0 | 6.205 |
| FCBBF2000362 | 8.370 | 0 | 0 | 17.080 | 0 | 0 | 0 |
| FCBBF2000566 | 0 | 8.527 | 0 | 0 | 0 | 0 | 0 |
| FCBBF2000591 | 0.287 | 0 | 0 | 0.292 | 0 | 0 | 0.318 |
| FCBBF2000685 | 0 | 15.608 | 0 | 0 | 25.321 | 0 | 0 |
| FCBBF2000733 | 31.702 | 0 | 0 | 16.172 | 29.499 | 0 | 0 |
| FCBBF2000885 | 9.210 | 0 | 0 | 6.264 | 0 | 0 | 1.702 |
| FCBBF2000940 | 0 | 0 | 0 | 0 | 0 | 0 | 37.510 |
| FCBBF2001001 | 19.115 | 0 | 0 | 0 | 0 | 34.461 | 0 |
| FCBBF2001105 | 3.035 | 0 | 10.753 | 0 | 0 | 0 | 0 |
| FCBBF2001183 | 16.537 | 3.794 | 0 | 11.810 | 6.155 | 0 | 9.169 |
| FCBBF2001211 | 25.634 | 14.703 | 0 | 0 | 0 | 0 | 7.107 |
| FCBBF2001291 | 1.902 | 0 | 0 | 1.941 | 0 | 0 | 0 |
| FCBBF2001400 | 0 | 0 | 0 | 0 | 0 | 0 | 22.584 |
| FCBBF2001480 | 0 | 55.394 | 0 | 0 | 0 | 0 | 0 |
| FCBBF2001538 | 0.762 | 1.748 | 0 | 2.332 | 5.672 | 0 | 3.380 |
| FCBBF2001594 | 7.606 | 0 | 0 | 11.640 | 0 | 0 | 21.086 |
| FCBBF2001817 | 0 | 55.394 | 0 | 0 | 0 | 0 | 0 |
| FCBBF2002349 | 0 | 0 | 13.985 | 4.028 | 0 | 0 | 0 |
| FCBBF2002801 | 5.191 | 0 | 9.194 | 2.648 | 0 | 0 | 0 |
| FCBBF2002928 | 0 | 0 | 0 | 3.174 | 0 | 0 | 0 |
| FCBBF2003293 | 0 | 0 | 0 | 0 | 0 | 66.122 | 0 |
| FCBBF2003543 | 35.119 | 0 | 0 | 0 | 0 | 0 | 0 |
| FCBBF2003567 | 0 | 0 | 0 | 19.952 | 0 | 0 | 7.229 |
| FCBBF2004066 | 4.032 | 0 | 7.142 | 6.170 | 0 | 29.076 | 11.178 |
| FCBBF2005909 | 6.241 | 0 | 0 | 19.102 | 0 | 0 | 6.921 |
| FCBBF2006634 | 0 | 10.829 | 0 | 0 | 0 | 0 | 0 |
| FCBBF2006781 | 0 | 0 | 0 | 0 | 0 | 0 | 7.017 |
| FCBBF3000115 | 11.973 | 0 | 0 | 8.143 | 0 | 0 | 0 |
| FCBBF3000168 | 8.142 | 18.679 | 0 | 8.306 | 0 | 0 | 9.029 |
| FCBBF3000228 | 0 | 0 | 0 | 9.143 | 0 | 0 | 0 |
| FCBBF3000229 | 0 | 22.152 | 0 | 0 | 35.938 | 0 | 0 |
| FCBBF3000388 | 9.880 | 11.334 | 0 | 0 | 0 | 17.812 | 0 |
| FCBBF3000434 | 1.851 | 8.495 | 0 | 11.333 | 6.891 | 0 | 0 |
| FCBBF3000536 | 0 | 0 | 9.443 | 0 | 0 | 0 | 0 |
| FCBBF3001081 | 0 | 0 | 0 | 0 | 0 | 0 | 5.824 |
| FCBBF3001377 | 10.297 | 0 | 0 | 0 | 0 | 0 | 0 |
| FCBBF3001470 | 0 | 0 | 0 | 0 | 0 | 0 | 3.011 |
| FCBBF3001632 | 0 | 3.542 | 0 | 0 | 0 | 0 | 0 |
| FCBBF3001657 | 11.795 | 0 | 0 | 9.627 | 8.780 | 0 | 5.232 |
| FCBBF3001890 | 0 | 0 | 19.489 | 0 | 0 | 0 | 3.050 |
| FCBBF3001977 | 7.448 | 0 | 0 | 7.599 | 0 | 0 | 0 |
| FCBBF3002818 | 35.119 | 0 | 0 | 0 | 0 | 0 | 0 |
| FCBBF3003557 | 35.119 | 0 | 0 | 0 | 0 | 0 | 0 |
| FCBBF3003787 | 9.787 | 0 | 0 | 0 | 0 | 0 | 0 |
| FCBBF3003800 | 2.363 | 1.807 | 1.395 | 2.813 | 2.932 | 1.420 | 1.310 |
| FCBBF3004323 | 5.510 | 0 | 0 | 0 | 0 | 0 | 0 |
| FCBBF3004473 | 2.298 | 0 | 0 | 1.172 | 0 | 0 | 2.548 |
| FCBBF3004715 | 3.745 | 0 | 0 | 0 | 0 | 0 | 0 |
| FCBBF3004955 | 0 | 0 | 0 | 23.589 | 0 | 0 | 0 |
| FCBBF3005320 | 0 | 0 | 0 | 5.566 | 0 | 0 | 0 |
| FCBBF3005330 | 2.771 | 0 | 2.454 | 2.827 | 0 | 0 | 1.536 |
| FCBBF3006821 | 5.716 | 0 | 0 | 5.832 | 0 | 0 | 0 |
| FCBBF3007462 | 0 | 0 | 0 | 35.577 | 0 | 0 | 0 |
| FCBBF3007631 | 21.846 | 0 | 0 | 22.288 | 0 | 0 | 0 |
| FCBBF3007859 | 12.992 | 0 | 0 | 13.255 | 0 | 0 | 7.204 |
| FCBBF3007860 | 50.592 | 0 | 0 | 10.323 | 0 | 0 | 0 |
| FCBBF3008153 | 10.942 | 0 | 0 | 0 | 0 | 0 | 6.067 |
| FCBBF3008159 | 0 | 0 | 0 | 21.128 | 0 | 0 | 0 |
| FCBBF3008644 | 8.616 | 0 | 0 | 0 | 0 | 0 | 0 |
| FCBBF3009069 | 17.913 | 0 | 0 | 4.569 | 0 | 0 | 34.763 |
| FCBBF3009101 | 35.119 | 0 | 0 | 0 | 0 | 0 | 0 |
| FCBBF3009152 | 19.020 | 0 | 0 | 0 | 0 | 0 | 0 |
| FCBBF3009479 | 0 | 0 | 0 | 0 | 0 | 0 | 10.970 |
| FCBBF3009526 | 11.814 | 0 | 0 | 0 | 0 | 0 | 13.101 |
| FCBBF3009541 | 0 | 0 | 0 | 1.262 | 0 | 0 | 0.914 |
| FCBBF3009978 | 0 | 0 | 65.725 | 0 | 0 | 0 | 0 |
| FCBBF3010012 | 6.894 | 5.272 | 0 | 2.344 | 0 | 8.285 | 0 |
| FCBBF3010142 | 14.433 | 0 | 0 | 14.725 | 0 | 0 | 16.005 |
| FCBBF3010695 | 4.960 | 0 | 0 | 0 | 0 | 0 | 2.750 |
| FCBBF3012332 | 21.300 | 0 | 0 | 0 | 0 | 0 | 0 |
| FCBBF3012347 | 0 | 0 | 7.775 | 0 | 8.169 | 0 | 2.434 |
| FCBBF3013266 | 0 | 0 | 0 | 0 | 0 | 0 | 23.444 |
| FCBBF3013506 | 23.748 | 0 | 0 | 0 | 0 | 0 | 0 |
| FCBBF3013800 | 0 | 0 | 0 | 35.577 | 0 | 0 | 0 |
| FCBBF3014260 | 2.258 | 0 | 7.999 | 11.518 | 0 | 0 | 0 |
| FCBBF3015100 | 4.990 | 0 | 17.676 | 0 | 0 | 0 | 5.533 |
| FCBBF3016622 | 35.119 | 0 | 0 | 0 | 0 | 0 | 0 |
| FCBBF3016667 | 0 | 0 | 0 | 13.060 | 0 | 0 | 0 |
| FCBBF3016928 | 2.629 | 0 | 0 | 1.788 | 0 | 0 | 3.887 |
| FCBBF3017255 | 0 | 0 | 0 | 35.577 | 0 | 0 | 0 |
| FCBBF3017396 | 0 | 0 | 0 | 6.248 | 0 | 0 | 0 |
| FCBBF3017531 | 3.207 | 3.678 | 0 | 0 | 0 | 0 | 3.556 |
| FCBBF3017918 | 6.547 | 0 | 0 | 0 | 0 | 0 | 0 |
| FCBBF3018067 | 0 | 0 | 0 | 0 | 0 | 66.122 | 0 |
| FCBBF3018173 | 0 | 0 | 0 | 35.577 | 0 | 0 | 0 |
| FCBBF3018796 | 0 | 7.122 | 0 | 0 | 0 | 0 | 6.886 |
| FCBBF3018949 | 0 | 0 | 0 | 14.438 | 0 | 0 | 15.693 |
| FCBBF3019839 | 14.980 | 0 | 0 | 11.462 | 0 | 0 | 4.153 |
| FCBBF3021026 | 0 | 55.394 | 0 | 0 | 0 | 0 | 0 |
| FCBBF3021807 | 0 | 0 | 0 | 0 | 0 | 15.634 | 0 |
| FCBBF3022566 | 35.119 | 0 | 0 | 0 | 0 | 0 | 0 |
| FCBBF3022593 | 0 | 0 | 0 | 0 | 0 | 0 | 7.683 |
| FCBBF3022767 | 0 | 0 | 0 | 0 | 0 | 0 | 22.584 |
| FCBBF3023368 | 7.361 | 0 | 0 | 2.503 | 0 | 0 | 2.721 |
| FCBBF3023667 | 0 | 0.305 | 0 | 0 | 0 | 0 | 0.736 |
| FCBBF3024002 | 5.675 | 0 | 0 | 0 | 0 | 0 | 0 |
| FCBBF3024266 | 9.782 | 0 | 0 | 9.980 | 0 | 0 | 0 |
| FCBBF3024623 | 0 | 0 | 0 | 0 | 0 | 0 | 12.426 |
| FCBBF3025285 | 0 | 55.394 | 0 | 0 | 0 | 0 | 0 |
| FCBBF3025417 | 0 | 0 | 0 | 0 | 31.948 | 0 | 19.038 |
| FCBBF3025528 | 0 | 0 | 0 | 4.926 | 0 | 0 | 0 |
| FCBBF3025568 | 16.446 | 0 | 0 | 33.557 | 0 | 0 | 0 |
| FCBBF3025650 | 0 | 0 | 0 | 25.656 | 0 | 0 | 27.887 |
| FCBBF3025737 | 0 | 0 | 0 | 0 | 0 | 11.719 | 3.604 |
| FCBBF3026308 | 0 | 29.322 | 0 | 0 | 0 | 0 | 14.174 |
| FCBBF3026651 | 14.594 | 0 | 0 | 22.334 | 0 | 0 | 8.092 |
| FCBBF3026692 | 0 | 0 | 0 | 0 | 0 | 58.507 | 0 |
| FCBBF3027559 | 35.119 | 0 | 0 | 0 | 0 | 0 | 0 |
| FCBBF3028244 | 5.571 | 2.130 | 3.289 | 2.842 | 0 | 3.348 | 1.030 |
| FCBBF3028593 | 0 | 0 | 0 | 0 | 0 | 0 | 8.955 |
| FCBBF4000142 | 3.656 | 0 | 0 | 0 | 0 | 0 | 4.054 |
| FCBBF4000268 | 0 | 0 | 0 | 35.577 | 0 | 0 | 0 |
| FCBBF4000446 | 2.253 | 1.723 | 0 | 0.766 | 0 | 2.708 | 0 |
| FCBBF5000061 | 0 | 0 | 0 | 0 | 0 | 0 | 23.231 |
| FCBBF5000165 | 9.875 | 0 | 0 | 23.508 | 0 | 0 | 0 |
| FCBBF5000261 | 0 | 0 | 0 | 0 | 0 | 0 | 11.516 |
| FCBBF5000325 | 0 | 23.194 | 0 | 10.314 | 37.628 | 0 | 0 |
| FEBRA1000022 | 0 | 0 | 0 | 10.739 | 0 | 0 | 0 |
| FEBRA1000057 | 1.736 | 7.966 | 6.150 | 3.542 | 6.461 | 0 | 1.925 |
| FEBRA1000088 | 0 | 9.791 | 0 | 0 | 0 | 0 | 0 |
| FEBRA1000188 | 0 | 0 | 0 | 2.135 | 0 | 0 | 0 |
| FEBRA1000189 | 0 | 0 | 0 | 20.683 | 0 | 0 | 0 |
| FEBRA1000190 | 0 | 18.233 | 0 | 0 | 0 | 0 | 0 |
| FEBRA2000007 | 0 | 0 | 0 | 24.160 | 0 | 0 | 0 |
| FEBRA2000053 | 21.156 | 0 | 0 | 7.195 | 0 | 0 | 23.461 |
| FEBRA2000105 | 0.717 | 1.645 | 0 | 0 | 0 | 0 | 4.772 |
| FEBRA2000129 | 5.199 | 5.964 | 0 | 0 | 0 | 0 | 2.883 |
| FEBRA2000210 | 0.707 | 0 | 0 | 0.721 | 0 | 0 | 0 |
| FEBRA2000253 | 0 | 0 | 0 | 14.934 | 0 | 0 | 0 |
| FEBRA2000297 | 0 | 18.194 | 0 | 0 | 0 | 0 | 8.794 |
| FEBRA2000333 | 0 | 0 | 0 | 0 | 0 | 0 | 30.789 |
| FEBRA2000402 | 0 | 0 | 0 | 5.655 | 0 | 0 | 0 |
| FEBRA2000452 | 4.012 | 0 | 0 | 2.046 | 0 | 0 | 3.337 |
| FEBRA2000462 | 31.051 | 0 | 0 | 8.640 | 0 | 0 | 6.261 |
| FEBRA2000536 | 0 | 0 | 0 | 3.214 | 0 | 0 | 3.493 |
| FEBRA2000538 | 0 | 0 | 0 | 0 | 59.888 | 0 | 0 |
| FEBRA2000581 | 21.479 | 0 | 0 | 15.421 | 0 | 0 | 7.940 |
| FEBRA2000680 | 0 | 0 | 0 | 9.589 | 0 | 0 | 0 |
| FEBRA2000733 | 0.506 | 0.387 | 1.194 | 0.860 | 1.254 | 0 | 0.934 |
| FEBRA2000740 | 2.262 | 0 | 0 | 1.154 | 0 | 0 | 1.254 |
| FEBRA2000757 | 12.249 | 0 | 0 | 21.870 | 0 | 0 | 6.792 |
| FEBRA2000762 | 0 | 0 | 0 | 0 | 0 | 22.430 | 0 |
| FEBRA2000771 | 0 | 0 | 0 | 0 | 0 | 0 | 24.058 |
| FEBRA2000772 | 0 | 19.625 | 0 | 0 | 0 | 0 | 9.486 |
| FEBRA2000787 | 0 | 0 | 6.997 | 0 | 0 | 0 | 2.190 |
| FEBRA2000793 | 0 | 0 | 0 | 0 | 0 | 0 | 4.253 |
| FEBRA2000815 | 0 | 0 | 12.807 | 0 | 0 | 0 | 0 |
| FEBRA2000877 | 14.227 | 0 | 0 | 0 | 0 | 0 | 15.777 |
| FEBRA2000880 | 0 | 24.311 | 0 | 0 | 0 | 0 | 0 |
| FEBRA2000901 | 0 | 0 | 0 | 0 | 0 | 37.454 | 0 |
| FEBRA2001004 | 0 | 0 | 0 | 22.568 | 0 | 0 | 0 |
| FEBRA2001146 | 11.183 | 0 | 0 | 0 | 0 | 0 | 0 |
| FEBRA2001197 | 0 | 0 | 0 | 0 | 0 | 0 | 15.632 |
| FEBRA2001227 | 3.697 | 0 | 13.098 | 11.316 | 0 | 0 | 12.300 |
| FEBRA2001245 | 12.612 | 0 | 8.936 | 10.293 | 0 | 0 | 8.391 |
| FEBRA2001294 | 0 | 0 | 8.561 | 0 | 0 | 0 | 2.680 |
| FEBRA2001334 | 11.501 | 0 | 0 | 5.867 | 0 | 0 | 0 |
| FEBRA2001440 | 0 | 0 | 0 | 0 | 0 | 41.969 | 0 |
| FEBRA2001492 | 22.656 | 0 | 0 | 6.604 | 0 | 0 | 14.357 |
| FEBRA2001571 | 0 | 0 | 0 | 2.140 | 0 | 0 | 4.651 |
| FEBRA2001581 | 0 | 9.407 | 0 | 0 | 15.261 | 0 | 0 |
| FEBRA2001591 | 0 | 0 | 0 | 0 | 0 | 0 | 11.328 |
| FEBRA2001669 | 17.140 | 0 | 0 | 20.766 | 0 | 0 | 4.752 |
| FEBRA2001698 | 12.173 | 0 | 0 | 12.420 | 0 | 0 | 0 |
| FEBRA2001706 | 28.630 | 0 | 0 | 0 | 0 | 0 | 0 |
| FEBRA2001715 | 3.989 | 0 | 0 | 25.433 | 0 | 0 | 6.635 |
| FEBRA2001745 | 0 | 0 | 4.440 | 0 | 0 | 0 | 0 |
| FEBRA2001814 | 3.969 | 3.035 | 0 | 2.700 | 0 | 0 | 1.467 |
| FEBRA2001867 | 0 | 0 | 13.129 | 0 | 0 | 0 | 12.330 |
| FEBRA2001974 | 0 | 26.731 | 0 | 0 | 0 | 0 | 0 |
| FEBRA2001990 | 18.144 | 16.650 | 0 | 7.404 | 0 | 0 | 8.048 |
| FEBRA2002027 | 2.533 | 0 | 0 | 2.585 | 9.430 | 9.135 | 0 |
| FEBRA2002352 | 0 | 0 | 12.297 | 3.542 | 0 | 0 | 0 |
| FEBRA2002442 | 0 | 47.925 | 0 | 0 | 0 | 0 | 0 |
| FEBRA2002456 | 13.088 | 0 | 0 | 8.902 | 0 | 0 | 14.515 |
| FEBRA2002611 | 21.730 | 0 | 0 | 0 | 0 | 0 | 24.098 |
| FEBRA2002707 | 3.603 | 0 | 0 | 0 | 0 | 0 | 0 |
| FEBRA2002781 | 6.842 | 0 | 0 | 0 | 0 | 0 | 0 |
| FEBRA2002792 | 8.031 | 9.212 | 0 | 10.925 | 9.964 | 4.826 | 16.328 |
| FEBRA2002931 | 18.604 | 0 | 3.877 | 16.748 | 0 | 0 | 15.777 |
| FEBRA2002933 | 0 | 0 | 0 | 0.755 | 2.755 | 0 | 0.821 |
| FEBRA2003100 | 0.659 | 0 | 0 | 2.016 | 0 | 0 | 4.382 |
| FEBRA2003115 | 21.678 | 0 | 0 | 0 | 0 | 0 | 0 |
| FEBRA2003128 | 31.997 | 0 | 0 | 14.839 | 0 | 0 | 6.452 |
| FEBRA2003258 | 0 | 0 | 0 | 2.886 | 0 | 10.201 | 6.275 |
| FEBRA2003436 | 0 | 0 | 39.387 | 0 | 0 | 0 | 0 |
| FEBRA2003470 | 14.595 | 0 | 0 | 0 | 0 | 0 | 16.185 |
| FEBRA2003707 | 0 | 47.925 | 0 | 0 | 0 | 0 | 0 |
| FEBRA2003726 | 0 | 2.285 | 0 | 0 | 0 | 0 | 1.104 |
| FEBRA2003750 | 0 | 0 | 12.934 | 1.862 | 0 | 0 | 6.073 |
| FEBRA2003833 | 5.187 | 0 | 6.126 | 7.057 | 0 | 0 | 1.918 |
| FEBRA2003926 | 0 | 0 | 2.473 | 0.712 | 0 | 2.517 | 0.774 |
| FEBRA2003930 | 0 | 0 | 0 | 0 | 6.689 | 0 | 1.993 |
| FEBRA2004042 | 0 | 0 | 0 | 2.164 | 0 | 0 | 0 |
| FEBRA2004091 | 10.568 | 0 | 37.438 | 0 | 0 | 0 | 0 |
| FEBRA2004219 | 0 | 0 | 0 | 29.041 | 0 | 0 | 0 |
| FEBRA2004224 | 5.789 | 0 | 0 | 7.087 | 8.618 | 8.349 | 2.568 |
| FEBRA2004293 | 0 | 6.099 | 0 | 0 | 0 | 19.171 | 2.948 |
| FEBRA2004325 | 0 | 0 | 0 | 0 | 0 | 0 | 7.812 |
| FEBRA2004485 | 0 | 0 | 0 | 0 | 0 | 0 | 7.696 |
| FEBRA2004490 | 14.095 | 0 | 0 | 0 | 0 | 0 | 15.631 |
| FEBRA2004592 | 0 | 0 | 0 | 0 | 59.888 | 0 | 0 |
| FEBRA2004628 | 0 | 0 | 0 | 0 | 31.851 | 0 | 0 |
| FEBRA2004818 | 0 | 6.248 | 0 | 0 | 5.068 | 0 | 0 |
| FEBRA2005291 | 0 | 0 | 0 | 0 | 0 | 13.076 | 0 |
| FEBRA2005361 | 8.405 | 19.282 | 12.406 | 2.858 | 5.214 | 0 | 0.777 |
| FEBRA2005416 | 2.148 | 0 | 3.804 | 1.096 | 3.997 | 0 | 2.382 |
| FEBRA2005427 | 28.630 | 0 | 0 | 0 | 0 | 0 | 0 |
| FEBRA2005476 | 0 | 0 | 0 | 0 | 0 | 0 | 2.888 |
| FEBRA2005572 | 1.782 | 0 | 0 | 0 | 0 | 0 | 5.928 |
| FEBRA2005752 | 0 | 0 | 0 | 0 | 0 | 15.008 | 0 |
| FEBRA2005788 | 24.677 | 0 | 0 | 5.035 | 0 | 0 | 0 |
| FEBRA2005824 | 3.730 | 1.711 | 9.249 | 12.558 | 13.883 | 4.035 | 8.686 |
| FEBRA2005995 | 2.804 | 0 | 4.966 | 0 | 0 | 0 | 6.218 |
| FEBRA2006055 | 0 | 6.060 | 0 | 0 | 0 | 0 | 0 |
| FEBRA2006092 | 3.109 | 1.427 | 0 | 0.634 | 0 | 0 | 1.379 |
| FEBRA2006255 | 14.333 | 0 | 4.616 | 9.306 | 4.850 | 4.698 | 10.115 |
| FEBRA2006270 | 0 | 0 | 0 | 0 | 0 | 0 | 1.296 |
| FEBRA2006357 | 11.922 | 0 | 0 | 34.464 | 2.465 | 0 | 4.407 |
| FEBRA2006368 | 3.107 | 14.258 | 0 | 3.170 | 0 | 0 | 3.446 |
| FEBRA2006370 | 0 | 0 | 0 | 14.576 | 0 | 0 | 0 |
| FEBRA2006372 | 0 | 0 | 0 | 1.370 | 0 | 0 | 1.489 |
| FEBRA2006401 | 0.502 | 0 | 0 | 0 | 0 | 0 | 0.557 |
| FEBRA2006485 | 3.042 | 0 | 0 | 0 | 0 | 10.968 | 0 |
| FEBRA2006519 | 11.891 | 0 | 0 | 0 | 0 | 0 | 0 |
| FEBRA2006521 | 7.648 | 0 | 0 | 0 | 0 | 0 | 16.963 |
| FEBRA2006553 | 3.956 | 0 | 0 | 0 | 0 | 0 | 0 |
| FEBRA2006627 | 0 | 0 | 0 | 10.526 | 0 | 0 | 0 |
| FEBRA2006808 | 0 | 0 | 14.542 | 8.376 | 0 | 0 | 0 |
| FEBRA2006817 | 0 | 0 | 0 | 0 | 0 | 59.124 | 0 |
| FEBRA2006890 | 44.515 | 0 | 0 | 0 | 0 | 0 | 0 |
| FEBRA2007176 | 28.630 | 0 | 0 | 0 | 0 | 0 | 0 |
| FEBRA2007212 | 0 | 0 | 0 | 16.019 | 0 | 0 | 4.353 |
| FEBRA2007534 | 0 | 0 | 0 | 36.825 | 0 | 0 | 0 |
| FEBRA2007544 | 14.689 | 0 | 0 | 4.995 | 0 | 0 | 2.715 |
| FEBRA2007551 | 0 | 0 | 0 | 0 | 0 | 0 | 30.789 |
| FEBRA2007589 | 13.674 | 0 | 0 | 20.927 | 0 | 0 | 15.164 |
| FEBRA2007613 | 3.318 | 0 | 11.756 | 6.771 | 12.351 | 11.965 | 0 |
| FEBRA2007622 | 0 | 0 | 0 | 1.053 | 3.840 | 0 | 0 |
| FEBRA2007801 | 4.937 | 0 | 8.745 | 7.556 | 0 | 0 | 5.475 |
| FEBRA2007818 | 0 | 0 | 0 | 0 | 0 | 0 | 6.201 |
| FEBRA2007823 | 28.630 | 0 | 0 | 0 | 0 | 0 | 0 |
| FEBRA2007880 | 0 | 0 | 0 | 3.840 | 0 | 0 | 4.174 |
| FEBRA2007900 | 7.751 | 0 | 0 | 0 | 0 | 0 | 8.595 |
| FEBRA2007901 | 0 | 8.503 | 0 | 7.562 | 0 | 0 | 0 |
| FEBRA2007957 | 54.616 | 0 | 0 | 0 | 0 | 0 | 0 |
| FEBRA2008036 | 4.285 | 3.277 | 0 | 4.372 | 5.316 | 0 | 4.752 |
| FEBRA2008081 | 2.124 | 3.249 | 0 | 0.722 | 2.636 | 0 | 0 |
| FEBRA2008086 | 0 | 0 | 0 | 0 | 0 | 0 | 30.789 |
| FEBRA2008087 | 0.358 | 0.822 | 0.635 | 0 | 0.667 | 0 | 0.596 |
| FEBRA2008201 | 2.398 | 0 | 0 | 0 | 0 | 0 | 2.659 |
| FEBRA2008210 | 0 | 0 | 23.761 | 0 | 0 | 0 | 0 |
| FEBRA2008255 | 0 | 23.219 | 0 | 0 | 0 | 0 | 0 |
| FEBRA2008266 | 0 | 0 | 0 | 0 | 0 | 0 | 10.077 |
| FEBRA2008287 | 0 | 0 | 0 | 3.007 | 0 | 0 | 6.537 |
| FEBRA2008302 | 0 | 7.325 | 0 | 1.086 | 0 | 11.512 | 3.540 |
| FEBRA2008360 | 0 | 0 | 0 | 0 | 0 | 0 | 11.338 |
| FEBRA2008583 | 0.925 | 0.606 | 0.936 | 1.483 | 0.984 | 1.430 | 3.371 |
| FEBRA2008662 | 11.110 | 0 | 0 | 3.239 | 0 | 11.446 | 1.760 |
| FEBRA2008755 | 0 | 0 | 5.819 | 0 | 0 | 0 | 0 |
| FEBRA2008861 | 21.424 | 0 | 0 | 3.974 | 0 | 7.023 | 4.320 |
| FEBRA2009022 | 0 | 0 | 0 | 0 | 12.430 | 0 | 0 |
| FEBRA2009185 | 4.543 | 6.253 | 3.219 | 8.342 | 10.145 | 3.276 | 0 |
| FEBRA2009289 | 0 | 0 | 0 | 14.316 | 0 | 0 | 0 |
| FEBRA2009328 | 4.955 | 11.368 | 0 | 0 | 0 | 0 | 0 |
| FEBRA2009352 | 0 | 0 | 0 | 29.041 | 0 | 0 | 0 |
| FEBRA2009362 | 7.614 | 0 | 26.973 | 0 | 0 | 27.453 | 0 |
| FEBRA2009419 | 5.285 | 3.031 | 0 | 2.696 | 0 | 0 | 4.396 |
| FEBRA2009514 | 0 | 0 | 0 | 3.379 | 0 | 0 | 0 |
| FEBRA2009588 | 19.382 | 0 | 0 | 0 | 0 | 0 | 5.373 |
| FEBRA2009846 | 0 | 0 | 0 | 0 | 0 | 0 | 22.893 |
| FEBRA2009986 | 6.427 | 0 | 0 | 19.673 | 0 | 0 | 7.128 |
| FEBRA2010719 | 0 | 0 | 0 | 17.703 | 0 | 0 | 0 |
| FEBRA2010746 | 8.579 | 0 | 0 | 0 | 0 | 0 | 0 |
| FEBRA2010768 | 0 | 0 | 0 | 0 | 0 | 59.124 | 0 |
| FEBRA2012181 | 16.511 | 0 | 0 | 16.845 | 0 | 0 | 0 |
| FEBRA2013699 | 7.157 | 0 | 0 | 7.301 | 0 | 0 | 0 |
| FEBRA2013951 | 0 | 0 | 0 | 0 | 0 | 59.124 | 0 |
| FEBRA2014297 | 0 | 14.710 | 7.571 | 2.180 | 0 | 7.706 | 0 |
| FEBRA2014417 | 7.559 | 6.194 | 3.826 | 3.856 | 4.019 | 5.841 | 1.198 |
| FEBRA2015085 | 1.525 | 0 | 0 | 1.556 | 0 | 5.498 | 5.072 |
| FEBRA2016398 | 17.280 | 39.644 | 0 | 0 | 0 | 0 | 0 |
| FEBRA2016572 | 0 | 0 | 0 | 0 | 0 | 0 | 21.790 |
| FEBRA2016583 | 0 | 0 | 0 | 29.041 | 0 | 0 | 0 |
| FEBRA2017024 | 0 | 0 | 0 | 0 | 0 | 0 | 21.399 |
| FEBRA2017533 | 5.455 | 0 | 0 | 5.566 | 0 | 0 | 0 |
| FEBRA2017680 | 0 | 21.815 | 0 | 9.701 | 0 | 0 | 0 |
| FEBRA2019242 | 0 | 0 | 0 | 8.277 | 0 | 0 | 0 |
| FEBRA2019711 | 0 | 4.851 | 0 | 0 | 0 | 0 | 2.345 |
| FEBRA2019867 | 0 | 0 | 0 | 0 | 0 | 0 | 30.789 |
| FEBRA2020845 | 28.630 | 0 | 0 | 0 | 0 | 0 | 0 |
| FEBRA2020886 | 12.124 | 0 | 0 | 12.369 | 0 | 0 | 0 |
| FEBRA2020977 | 0 | 0 | 0 | 5.376 | 19.613 | 0 | 0 |
| FEBRA2021171 | 0 | 24.406 | 0 | 0 | 0 | 38.357 | 0 |
| FEBRA2022013 | 0 | 0 | 0 | 0 | 0 | 0 | 10.426 |
| FEBRA2022055 | 0 | 1.221 | 0 | 0 | 0 | 0 | 0 |
| FEBRA2022504 | 0 | 3.768 | 11.636 | 0 | 0 | 0 | 0 |
| FEBRA2022696 | 0 | 47.925 | 0 | 0 | 0 | 0 | 0 |
| FEBRA2023227 | 0 | 0 | 0 | 0 | 0 | 0 | 5.680 |
| FEBRA2023377 | 15.515 | 0 | 27.481 | 0 | 0 | 0 | 0 |
| FEBRA2023927 | 4.073 | 0 | 0 | 8.311 | 0 | 0 | 9.033 |
| FEBRA2024019 | 5.364 | 6.153 | 4.751 | 4.105 | 14.974 | 9.671 | 2.974 |
| FEBRA2024136 | 0 | 0 | 0 | 0 | 42.701 | 0 | 0 |
| FEBRA2024570 | 0 | 0 | 0 | 0 | 74.913 | 0 | 0 |
| FEBRA2025427 | 0 | 0 | 0 | 0 | 9.226 | 0 | 0 |
| FEBRA2025838 | 0.821 | 1.885 | 0 | 2.514 | 0 | 2.962 | 0.911 |
| FEBRA2028503 | 0 | 0 | 0 | 0 | 52.044 | 0 | 7.753 |
| FEBRA2028516 | 8.007 | 0 | 0 | 4.668 | 0 | 0 | 1.268 |
| FELIV1000153 | 0 | 0.378 | 0 | 0.168 | 0 | 0 | 0.183 |
| HCASM1000115 | 2.073 | 0 | 0 | 5.287 | 0 | 0 | 0 |
| HCASM2000016 | 1.493 | 3.425 | 0 | 2.284 | 0 | 0 | 1.655 |
| HCASM2000138 | 12.671 | 0 | 0 | 4.309 | 0 | 0 | 4.684 |
| HCASM2000214 | 0 | 0 | 0 | 0 | 0 | 0 | 10.488 |
| HCASM2000363 | 0 | 1.560 | 0 | 0 | 0 | 2.452 | 3.017 |
| HCASM2003076 | 0 | 0 | 0 | 0 | 0 | 0 | 6.314 |
| HCASM2005388 | 3.537 | 0 | 0 | 3.609 | 0 | 0 | 0 |
| HCASM2005484 | 0 | 0 | 0 | 0 | 0 | 0 | 14.312 |
| HCASM2007047 | 3.431 | 0 | 0 | 0 | 0 | 0 | 0 |
| HCHON1000015 | 0 | 0 | 0 | 1.590 | 0 | 0 | 0 |
| HCHON1000030 | 0.726 | 0.832 | 0.643 | 0.555 | 2.025 | 0.654 | 0.603 |
| HCHON1000131 | 4.103 | 0 | 0 | 4.186 | 0 | 0 | 0 |
| HCHON1000166 | 0 | 0 | 10.657 | 0 | 0 | 0 | 3.336 |
| HCHON1000191 | 0 | 0 | 0 | 0 | 0 | 0 | 1.071 |
| HCHON2000028 | 0 | 0 | 5.620 | 1.619 | 0 | 0 | 5.278 |
| HCHON2000056 | 0.567 | 1.301 | 0 | 0 | 0 | 3.068 | 2.202 |
| HCHON2000062 | 0 | 0 | 0 | 0 | 0 | 0 | 4.648 |
| HCHON2000087 | 0.619 | 0 | 0 | 0 | 0 | 0 | 0.686 |
| HCHON2000160 | 0 | 6.928 | 0 | 2.054 | 0 | 0 | 2.233 |
| HCHON2000212 | 0 | 0 | 0 | 0 | 0 | 0 | 19.062 |
| HCHON2000226 | 0.403 | 0.925 | 2.857 | 0.411 | 0 | 1.454 | 0 |
| HCHON2000244 | 0.658 | 0 | 0 | 0.671 | 0 | 0 | 0 |
| HCHON2000295 | 0 | 1.005 | 0 | 0.447 | 0 | 0 | 0 |
| HCHON2000322 | 10.980 | 0 | 0 | 0 | 0 | 0 | 0 |
| HCHON2000364 | 0 | 0 | 0 | 0 | 0 | 0 | 0.493 |
| HCHON2000473 | 0 | 0 | 0 | 5.972 | 0 | 0 | 6.491 |
| HCHON2000475 | 0 | 3.054 | 0 | 0 | 0 | 0 | 1.476 |
| HCHON2000626 | 2.351 | 8.091 | 4.164 | 0 | 4.375 | 0 | 1.304 |
| HCHON2000663 | 0 | 0 | 0 | 8.703 | 0 | 0 | 18.920 |
| HCHON2000698 | 0 | 13.152 | 0 | 2.924 | 0 | 0 | 3.179 |
| HCHON2000738 | 0 | 0 | 0 | 0 | 11.082 | 0 | 0 |
| HCHON2000743 | 6.105 | 0 | 0 | 0 | 0 | 0 | 6.771 |
| HCHON2000751 | 3.570 | 0 | 0 | 0 | 0 | 0 | 3.959 |
| HCHON2000815 | 2.793 | 0 | 0 | 0 | 10.394 | 0 | 0 |
| HCHON2000826 | 3.578 | 0 | 0 | 3.651 | 2.664 | 0 | 3.968 |
| HCHON2001039 | 1.116 | 0 | 0 | 2.277 | 4.153 | 0 | 1.237 |
| HCHON2001084 | 0 | 5.268 | 0 | 0 | 0 | 0 | 2.546 |
| HCHON2001099 | 0 | 0 | 0 | 2.783 | 0 | 0 | 3.025 |
| HCHON2001108 | 0 | 4.899 | 0 | 0 | 0 | 0 | 0 |
| HCHON2001200 | 0.433 | 0 | 0 | 1.324 | 0 | 0 | 1.919 |
| HCHON2001217 | 3.123 | 0 | 0 | 2.124 | 0 | 0 | 5.771 |
| HCHON2001359 | 1.703 | 0 | 3.016 | 2.606 | 0 | 0 | 0.944 |
| HCHON2001407 | 0 | 1.812 | 0 | 0 | 2.939 | 0 | 1.751 |
| HCHON2001434 | 13.748 | 3.505 | 5.412 | 1.558 | 0 | 5.508 | 5.082 |
| HCHON2001535 | 18.444 | 0 | 0 | 0 | 0 | 0 | 0 |
| HCHON2001665 | 0 | 0 | 0 | 6.297 | 0 | 0 | 0 |
| HCHON2001801 | 0 | 0 | 35.892 | 0 | 0 | 0 | 0 |
| HCHON2001853 | 0 | 0 | 0 | 11.428 | 0 | 0 | 0 |
| HCHON2002247 | 5.615 | 0 | 9.945 | 2.864 | 0 | 0 | 9.340 |
| HCHON2002676 | 13.647 | 0 | 0 | 0 | 0 | 0 | 0 |
| HCHON2004002 | 0 | 0 | 0 | 0 | 0 | 0 | 1.741 |
| HCHON2005921 | 0 | 0 | 0 | 12.694 | 0 | 0 | 9.198 |
| HCHON2006250 | 1.565 | 0 | 0 | 0 | 0 | 0 | 1.735 |
| HCHON2006722 | 0 | 0 | 35.892 | 0 | 0 | 0 | 0 |
| HCHON2006770 | 2.822 | 0 | 0 | 0 | 0 | 0 | 0 |
| HCHON2006786 | 0 | 0 | 0 | 13.885 | 0 | 0 | 0 |
| HCHON2010543 | 0 | 0 | 0 | 9.152 | 0 | 0 | 0 |
| HEART1000074 | 0.759 | 1.742 | 0 | 0 | 0 | 0 | 0 |
| HEART1000118 | 0 | 0 | 0 | 0 | 0 | 0 | 12.291 |
| HEART1000142 | 0 | 0 | 0 | 1.819 | 0 | 0 | 0 |
| HEART1000149 | 0 | 0 | 0 | 0.958 | 0 | 0 | 1.041 |
| HEART1000185 | 0.791 | 0 | 0 | 0 | 0 | 0 | 0 |
| HEART2000444 | 0 | 0 | 0 | 8.592 | 0 | 0 | 0 |
| HEART2000541 | 0 | 3.070 | 4.740 | 0 | 0 | 0 | 0 |
| HEART2000959 | 0 | 0 | 0 | 0 | 0 | 0 | 13.062 |
| HEART2003168 | 0 | 0 | 0 | 0 | 0 | 0 | 14.253 |
| HEART2003852 | 0 | 0 | 0 | 0 | 14.501 | 14.048 | 0 |
| HEART2006195 | 0 | 0 | 0 | 9.669 | 0 | 0 | 0 |
| HEART2006521 | 0 | 5.487 | 0 | 0 | 0 | 0 | 2.652 |
| HEART2007031 | 0 | 0 | 0 | 11.593 | 0 | 0 | 12.601 |
| HEART2007231 | 0 | 0 | 0 | 4.942 | 0 | 0 | 0 |
| HEART2007696 | 0 | 0 | 0 | 0 | 0 | 17.959 | 0 |
| HEART2008509 | 0 | 0 | 0 | 0 | 0 | 21.275 | 0 |
| HEART2009000 | 0 | 23.712 | 0 | 0 | 0 | 0 | 0 |
| HHDPC1000065 | 0 | 0 | 0 | 27.988 | 0 | 0 | 0 |
| HHDPC1000083 | 0 | 2.246 | 0 | 0 | 3.643 | 0 | 2.171 |
| HHDPC1000114 | 2.906 | 0 | 0 | 1.483 | 2.704 | 0 | 3.223 |
| HHDPC1000118 | 2.096 | 0 | 0 | 6.415 | 0 | 0 | 0 |
| HHDPC1000163 | 0 | 0 | 4.455 | 0 | 0 | 4.534 | 0 |
| HHDPC2000055 | 0 | 0 | 6.478 | 1.866 | 0 | 0 | 0 |
| HHDPC2000104 | 0 | 0 | 0 | 0 | 0 | 0 | 2.052 |
| HHDPC2000115 | 0 | 6.002 | 0 | 0 | 0 | 0 | 2.901 |
| HHDPC2000149 | 0 | 0 | 0 | 0 | 0 | 0 | 4.955 |
| HHDPC2000258 | 0 | 0 | 33.472 | 0 | 0 | 0 | 0 |
| HHDPC2000455 | 0 | 0 | 0 | 1.121 | 0 | 0 | 0 |
| HHDPC2000456 | 0 | 0 | 0 | 1.584 | 0 | 5.598 | 0 |
| HHDPC2003439 | 1.780 | 0 | 0 | 1.816 | 0.947 | 0 | 0.846 |
| HHDPC2003983 | 1.671 | 0 | 0.986 | 0.852 | 0 | 0 | 1.544 |
| HHDPC2005794 | 0 | 0 | 0 | 8.116 | 0 | 0 | 8.821 |
| HHDPC2007267 | 0 | 0 | 0 | 0 | 0 | 0 | 1.083 |
| HHDPC2007775 | 0 | 0 | 0 | 11.025 | 0 | 0 | 0 |
| HHDPC2008816 | 0 | 0 | 0 | 0 | 0 | 0 | 13.606 |
| HHDPC2009528 | 0 | 0 | 0 | 6.631 | 0 | 0 | 14.415 |
| HLUNG1000055 | 0 | 0 | 1.918 | 1.105 | 4.029 | 1.952 | 1.201 |
| HLUNG2000063 | 2.466 | 0 | 0 | 2.516 | 0 | 0 | 2.735 |
| HLUNG2000232 | 12.603 | 0 | 0 | 0 | 0 | 0 | 0 |
| HLUNG2000314 | 0 | 0 | 0 | 5.006 | 0 | 8.846 | 0 |
| HLUNG2000315 | 0 | 0 | 0 | 0 | 0 | 0 | 16.914 |
| HLUNG2000412 | 0 | 0 | 0 | 0 | 0 | 0 | 2.969 |
| HLUNG2000501 | 0.858 | 5.904 | 0 | 1.750 | 3.193 | 3.093 | 0 |
| HLUNG2000751 | 17.277 | 0 | 0 | 0 | 0 | 0 | 0 |
| HLUNG2000846 | 22.589 | 0 | 0 | 32.264 | 0 | 0 | 5.010 |
| HLUNG2000884 | 0 | 0 | 15.166 | 2.184 | 0 | 7.718 | 0 |
| HLUNG2001146 | 5.242 | 12.026 | 0 | 0 | 0 | 0 | 5.813 |
| HLUNG2001439 | 0 | 0 | 0 | 0 | 0 | 0 | 2.934 |
| HLUNG2001459 | 0 | 15.036 | 0 | 0 | 0 | 0 | 0 |
| HLUNG2001518 | 4.374 | 0 | 0 | 5.578 | 8.140 | 0 | 3.638 |
| HLUNG2001996 | 0 | 0 | 0 | 0 | 0 | 0 | 23.216 |
| HLUNG2002465 | 1.209 | 2.775 | 4.285 | 3.702 | 0 | 0 | 1.341 |
| HLUNG2002648 | 0 | 8.292 | 0 | 0 | 0 | 0 | 8.016 |
| HLUNG2002811 | 7.310 | 0 | 0 | 2.486 | 0 | 0 | 2.702 |
| HLUNG2002916 | 0 | 0 | 0 | 8.802 | 0 | 0 | 9.567 |
| HLUNG2003003 | 0 | 0 | 0 | 16.306 | 0 | 0 | 0 |
| HLUNG2003042 | 0 | 0 | 0 | 2.429 | 0 | 0 | 0 |
| HLUNG2004037 | 0 | 0 | 0 | 15.575 | 0 | 0 | 0 |
| HLUNG2004067 | 0 | 25.262 | 0 | 0 | 0 | 0 | 0 |
| HLUNG2004159 | 0 | 4.159 | 0 | 0 | 0 | 0 | 2.011 |
| HLUNG2004170 | 12.324 | 0 | 0 | 0 | 0 | 0 | 0 |
| HLUNG2004534 | 0 | 0 | 0 | 2.959 | 0 | 0 | 0 |
| HLUNG2005230 | 0 | 0 | 0 | 0 | 0 | 0 | 7.605 |
| HLUNG2006570 | 0 | 0 | 0 | 0 | 0 | 0 | 7.167 |
| HLUNG2006812 | 0 | 0 | 0 | 0 | 0 | 0 | 6.558 |
| HLUNG2008139 | 11.461 | 0 | 0 | 23.386 | 0 | 0 | 0 |
| HLUNG2008153 | 0 | 0 | 24.114 | 0 | 0 | 0 | 0 |
| HLUNG2009067 | 0 | 0 | 0 | 0 | 0 | 0 | 23.216 |
| HLUNG2009225 | 6.033 | 0 | 0 | 1.539 | 11.226 | 0 | 5.017 |
| HLUNG2011461 | 21.423 | 0 | 0 | 0 | 0 | 0 | 0 |
| HLUNG2015184 | 0 | 0 | 0 | 0 | 0 | 0 | 14.521 |
| HLUNG2017262 | 1.586 | 0 | 0 | 6.472 | 0 | 0 | 5.276 |
| HSYRA1000062 | 0.857 | 0 | 0 | 0 | 3.188 | 0 | 0.950 |
| HSYRA1000119 | 1.839 | 2.110 | 1.629 | 2.815 | 3.423 | 0 | 0 |
| HSYRA1000152 | 1.193 | 0 | 0 | 1.217 | 0 | 0 | 1.323 |
| HSYRA1000178 | 0 | 0 | 0 | 2.558 | 0 | 0 | 2.781 |
| HSYRA2000137 | 0 | 0 | 0 | 0 | 16.873 | 16.346 | 0 |
| HSYRA2000159 | 9.163 | 2.489 | 1.281 | 7.011 | 1.346 | 1.304 | 3.343 |
| HSYRA2000190 | 0 | 0 | 0 | 0 | 0 | 8.564 | 0 |
| HSYRA2000224 | 5.394 | 0 | 0 | 3.669 | 0 | 0 | 0 |
| HSYRA2000232 | 2.958 | 1.696 | 5.239 | 0 | 0 | 0 | 0 |
| HSYRA2000248 | 0.680 | 0 | 0 | 1.387 | 0 | 0 | 3.768 |
| HSYRA2000255 | 0 | 0 | 0 | 0.458 | 0 | 0 | 0 |
| HSYRA2000332 | 0 | 0 | 0 | 0.796 | 0 | 0 | 0 |
| HSYRA2000629 | 0 | 7.657 | 0 | 0 | 0 | 0 | 0 |
| HSYRA2000640 | 0 | 0 | 0 | 2.607 | 0 | 0 | 3.779 |
| HSYRA2000743 | 4.643 | 0 | 0 | 5.527 | 2.880 | 0 | 4.291 |
| HSYRA2000787 | 0 | 7.136 | 0 | 0 | 11.577 | 11.215 | 3.449 |
| HSYRA2000828 | 1.425 | 3.268 | 0 | 1.453 | 0 | 5.137 | 0 |
| HSYRA2000925 | 0.443 | 0.508 | 0 | 0.226 | 0 | 0 | 0.737 |
| HSYRA2001105 | 16.531 | 0 | 0 | 0 | 10.255 | 0 | 3.055 |
| HSYRA2001138 | 1.020 | 1.755 | 1.806 | 1.040 | 5.693 | 0.919 | 2.262 |
| HSYRA2001232 | 0 | 0 | 0 | 0 | 0 | 0 | 4.290 |
| HSYRA2001353 | 0 | 0 | 0 | 0 | 18.885 | 0 | 5.627 |
| HSYRA2001396 | 1.519 | 0 | 5.382 | 1.550 | 0 | 5.478 | 3.370 |
| HSYRA2001476 | 0 | 0 | 1.710 | 0 | 0 | 0 | 0.535 |
| HSYRA2001484 | 0 | 0 | 0 | 0 | 0 | 32.543 | 0 |
| HSYRA2001574 | 0.752 | 0 | 2.666 | 0.768 | 2.800 | 0 | 0 |
| HSYRA2001595 | 0.570 | 1.307 | 0 | 0 | 0 | 2.054 | 0 |
| HSYRA2001615 | 4.148 | 0 | 0 | 4.232 | 0 | 0 | 2.300 |
| HSYRA2001621 | 0 | 4.210 | 0.650 | 0 | 2.049 | 1.323 | 0.203 |
| HSYRA2001638 | 0 | 0 | 0 | 2.307 | 0 | 8.155 | 0 |
| HSYRA2001983 | 0 | 0 | 0 | 0 | 0 | 0 | 5.352 |
| HSYRA2005628 | 0 | 0 | 0 | 0 | 0 | 0 | 7.437 |
| IMR321000158 | 0 | 3.493 | 0 | 0.777 | 2.833 | 2.745 | 1.688 |
| IMR321000210 | 1.014 | 4.651 | 0 | 1.034 | 0 | 0 | 1.124 |
| IMR321000219 | 5.894 | 0 | 0 | 8.877 | 2.089 | 0 | 3.113 |
| IMR321000242 | 0 | 0 | 0 | 0 | 20.930 | 0 | 0 |
| IMR321000253 | 0 | 0 | 3.608 | 0 | 0 | 0 | 1.129 |
| IMR321000266 | 5.660 | 6.492 | 0 | 0 | 0 | 0 | 0 |
| IMR322000072 | 0.802 | 0 | 0 | 0 | 0 | 2.893 | 0 |
| IMR322000121 | 0 | 0 | 0 | 1.137 | 4.149 | 0 | 0 |
| IMR322000127 | 3.098 | 0 | 5.488 | 3.161 | 0 | 0 | 0 |
| IMR322000144 | 0 | 0 | 0 | 0 | 6.071 | 5.881 | 0 |
| IMR322000152 | 0 | 0 | 0 | 8.265 | 0 | 0 | 0 |
| IMR322000223 | 0.681 | 0 | 0 | 0 | 0 | 2.455 | 0 |
| IMR322000243 | 0 | 0 | 0 | 2.626 | 0 | 0 | 0.951 |
| IMR322000302 | 0 | 0 | 0 | 6.053 | 0 | 0 | 0 |
| IMR322000316 | 5.394 | 0.952 | 0 | 5.080 | 1.544 | 8.977 | 4.602 |
| IMR322000518 | 6.142 | 0 | 0 | 0 | 0 | 22.148 | 0 |
| IMR322000555 | 5.966 | 0 | 0 | 0 | 0 | 0 | 6.616 |
| IMR322000604 | 22.082 | 0 | 0 | 0 | 0 | 0 | 0 |
| IMR322000609 | 22.082 | 0 | 0 | 0 | 0 | 0 | 0 |
| IMR322000730 | 0 | 0.943 | 0 | 0.140 | 0 | 0.494 | 0.456 |
| IMR322000775 | 4.146 | 0 | 0 | 0 | 0 | 0 | 18.391 |
| IMR322000819 | 0 | 0 | 0 | 0 | 15.864 | 0 | 0 |
| IMR322000838 | 0 | 0 | 0 | 0 | 0 | 0 | 23.913 |
| IMR322000918 | 0 | 0 | 10.384 | 2.991 | 10.910 | 0 | 0 |
| IMR322000935 | 0 | 0 | 0 | 4.582 | 16.717 | 0 | 0 |
| IMR322001185 | 0 | 0 | 0 | 0 | 0 | 50.541 | 0 |
| IMR322001218 | 0 | 0 | 0 | 0 | 0 | 4.037 | 0 |
| IMR322001317 | 0.790 | 1.812 | 0 | 0 | 0 | 11.389 | 0 |
| IMR322001332 | 0 | 0 | 0 | 0 | 0 | 9.600 | 0 |
| IMR322001380 | 0 | 9.096 | 0 | 2.022 | 0 | 21.443 | 2.198 |
| IMR322001435 | 0.939 | 2.155 | 0 | 0.958 | 0 | 0 | 3.126 |
| IMR322001541 | 1.692 | 0 | 0 | 0 | 0 | 6.100 | 0 |
| IMR322001584 | 2.497 | 0 | 0 | 1.274 | 0 | 0 | 0 |
| IMR322001600 | 0 | 0 | 0 | 0.329 | 0 | 0.581 | 0 |
| IMR322001710 | 1.939 | 4.450 | 0 | 0 | 0 | 0 | 0 |
| IMR322001879 | 9.638 | 0 | 0 | 9.833 | 0 | 0 | 0 |
| IMR322002035 | 36.176 | 0 | 0 | 0 | 0 | 0 | 0 |
| IMR322002993 | 15.565 | 0 | 0 | 0 | 0 | 0 | 0 |
| IMR322013805 | 13.508 | 0 | 0 | 27.563 | 0 | 0 | 0 |
| IMR322019070 | 0 | 0 | 0 | 8.189 | 0 | 0 | 2.967 |
| JCMLC2000273 | 0 | 7.090 | 0 | 0 | 0 | 0 | 0 |
| KIDNE1000104 | 16.584 | 0 | 0 | 0 | 0 | 0 | 0 |
| KIDNE1000145 | 2.765 | 0 | 0 | 0 | 0 | 0 | 0 |
| KIDNE2000085 | 1.028 | 0 | 0 | 0 | 3.827 | 0 | 0 |
| KIDNE2000252 | 6.837 | 0 | 6.055 | 3.488 | 0 | 3.082 | 2.843 |
| KIDNE2000266 | 4.210 | 0 | 0 | 4.296 | 0 | 0 | 0 |
| KIDNE2000315 | 6.829 | 0 | 0 | 0 | 0 | 0 | 0 |
| KIDNE2000317 | 0 | 0 | 50.400 | 0 | 0 | 0 | 0 |
| KIDNE2000330 | 0 | 0 | 0 | 0 | 0 | 0 | 3.786 |
| KIDNE2000383 | 0 | 0 | 17.446 | 5.024 | 0 | 0 | 5.461 |
| KIDNE2000403 | 0.826 | 0 | 0 | 0 | 0 | 0 | 0 |
| KIDNE2000422 | 22.290 | 0 | 0 | 0 | 0 | 0 | 0 |
| KIDNE2000497 | 0 | 0 | 0 | 0 | 0 | 0 | 3.477 |
| KIDNE2000517 | 0 | 7.186 | 16.644 | 4.793 | 0 | 11.294 | 0 |
| KIDNE2000574 | 0.749 | 0 | 2.652 | 2.291 | 0 | 8.097 | 0 |
| KIDNE2000909 | 0 | 4.710 | 0 | 0 | 0 | 0 | 2.277 |
| KIDNE2000947 | 2.527 | 0 | 0 | 0 | 0 | 0 | 2.803 |
| KIDNE2001117 | 22.290 | 0 | 0 | 0 | 0 | 0 | 0 |
| KIDNE2001162 | 1.400 | 2.142 | 0 | 0 | 1.737 | 1.683 | 0 |
| KIDNE2001467 | 1.825 | 1.196 | 0 | 2.659 | 0.970 | 0.940 | 3.179 |
| KIDNE2001636 | 0 | 0 | 0 | 0 | 0 | 0 | 5.240 |
| KIDNE2002191 | 0 | 0 | 0 | 0 | 0 | 0 | 7.905 |
| KIDNE2002252 | 0 | 0 | 0 | 0 | 0 | 0 | 2.417 |
| KIDNE2002725 | 10.289 | 23.605 | 0 | 0 | 0 | 0 | 11.410 |
| KIDNE2002798 | 0 | 0 | 0 | 0 | 0 | 0 | 13.421 |
| KIDNE2002883 | 0 | 5.203 | 0 | 0 | 8.442 | 8.178 | 0 |
| KIDNE2003305 | 0 | 0 | 0 | 0 | 0 | 0 | 0.933 |
| KIDNE2003941 | 0.504 | 0 | 0 | 0.515 | 1.877 | 0 | 0.559 |
| KIDNE2004475 | 0 | 0 | 0 | 2.626 | 0 | 9.281 | 0 |
| KIDNE2005042 | 8.051 | 0 | 28.522 | 16.428 | 0 | 0 | 8.928 |
| KIDNE2005543 | 0 | 0 | 0 | 18.168 | 0 | 0 | 19.748 |
| KIDNE2005676 | 9.848 | 0 | 0 | 1.435 | 0 | 0 | 1.560 |
| KIDNE2005937 | 0 | 0 | 0 | 5.529 | 0 | 0 | 0 |
| KIDNE2006009 | 0 | 0 | 0 | 0 | 0 | 13.293 | 0 |
| KIDNE2006014 | 0 | 0 | 0 | 3.029 | 0 | 0 | 0 |
| KIDNE2006030 | 8.536 | 4.896 | 15.120 | 6.532 | 0 | 0 | 9.466 |
| KIDNE2006039 | 0 | 0 | 0 | 0 | 0 | 0 | 17.506 |
| KIDNE2006248 | 0.311 | 0.713 | 1.101 | 0 | 0 | 2.242 | 1.034 |
| KIDNE2006465 | 3.094 | 0 | 0 | 0 | 0 | 0 | 3.431 |
| KIDNE2006580 | 7.013 | 0 | 0 | 14.310 | 0 | 0 | 7.777 |
| KIDNE2006760 | 0 | 15.476 | 0 | 0 | 0 | 0 | 7.481 |
| KIDNE2007328 | 0.885 | 0 | 0 | 4.513 | 0 | 0 | 0.981 |
| KIDNE2007352 | 5.404 | 0.954 | 1.473 | 2.121 | 0 | 0 | 0.922 |
| KIDNE2007954 | 6.324 | 0 | 0 | 0 | 0 | 7.601 | 4.675 |
| KIDNE2008048 | 2.682 | 0 | 1.900 | 4.378 | 0 | 0 | 2.380 |
| KIDNE2008069 | 2.334 | 5.356 | 24.810 | 3.573 | 26.066 | 8.417 | 0 |
| KIDNE2008362 | 4.566 | 0 | 0 | 13.976 | 0 | 32.928 | 0 |
| KIDNE2008403 | 23.120 | 3.120 | 0 | 22.200 | 0 | 0 | 1.508 |
| KIDNE2008404 | 0 | 0 | 0 | 0 | 0 | 0 | 5.142 |
| KIDNE2008666 | 22.290 | 0 | 0 | 0 | 0 | 0 | 0 |
| KIDNE2008758 | 0 | 0 | 0 | 0 | 0 | 0 | 24.133 |
| KIDNE2008914 | 0 | 0 | 0 | 17.747 | 0 | 0 | 0 |
| KIDNE2008987 | 0 | 6.609 | 0 | 0 | 0 | 0 | 0 |
| KIDNE2009367 | 12.812 | 1.729 | 0 | 19.992 | 2.805 | 0 | 10.030 |
| KIDNE2009605 | 0 | 0 | 0 | 0 | 0 | 0 | 16.747 |
| KIDNE2010049 | 3.438 | 0 | 0 | 0 | 0 | 0 | 0 |
| KIDNE2010265 | 2.363 | 0 | 0 | 0 | 0 | 0 | 0 |
| KIDNE2010430 | 0 | 21.878 | 0 | 0 | 0 | 0 | 10.575 |
| KIDNE2010750 | 0 | 0 | 0 | 0 | 0 | 0 | 11.811 |
| KIDNE2011314 | 0 | 0 | 0 | 0 | 0 | 0 | 24.133 |
| KIDNE2012440 | 0 | 0 | 0 | 3.612 | 6.588 | 0 | 3.926 |
| KIDNE2012710 | 0 | 0 | 0 | 0 | 0 | 0 | 10.501 |
| KIDNE2013845 | 2.235 | 0 | 0 | 1.140 | 4.160 | 0 | 2.479 |
| KIDNE2014290 | 0 | 0 | 0 | 0 | 0 | 0 | 0.777 |
| KIDNE2014320 | 0 | 0 | 0 | 0 | 0 | 0 | 24.133 |
| KIDNE2015433 | 26.508 | 0 | 0 | 0 | 32.888 | 0 | 9.799 |
| KIDNE2015445 | 0 | 0 | 12.171 | 3.505 | 0 | 0 | 0 |
| KIDNE2016327 | 0 | 0 | 0 | 0 | 0 | 0 | 19.403 |
| KIDNE2016371 | 0 | 0 | 0 | 9.192 | 0 | 0 | 9.991 |
| KIDNE2017040 | 0 | 0 | 0 | 6.861 | 0 | 0 | 0 |
| KIDNE2017343 | 0 | 0 | 0 | 0 | 0 | 0 | 7.325 |
| KIDNE2018352 | 0 | 0 | 0 | 3.638 | 0 | 0 | 11.864 |
| KIDNE2018493 | 0 | 0 | 0 | 0 | 13.262 | 0 | 0 |
| LIVER1000017 | 0 | 0 | 0.527 | 0 | 0 | 0 | 0 |
| LIVER1000079 | 0 | 0 | 0 | 0 | 6.522 | 0 | 0 |
| LIVER1000104 | 0 | 0 | 0.730 | 0 | 0 | 0 | 0.457 |
| LIVER1000126 | 0 | 0 | 0 | 0 | 28.534 | 0 | 0 |
| LIVER1000132 | 0 | 0 | 0 | 0 | 1.862 | 0 | 3.330 |
| LIVER1000230 | 0 | 0 | 20.544 | 0 | 0 | 0 | 0 |
| LIVER2000033 | 0 | 0 | 4.901 | 0 | 5.149 | 0 | 0 |
| LIVER2000416 | 2.263 | 0 | 0 | 2.308 | 0 | 8.158 | 0 |
| LIVER2001608 | 0 | 0 | 0 | 0 | 0 | 0 | 2.913 |
| LIVER2001835 | 0 | 0 | 29.011 | 0 | 0 | 0 | 0 |
| LIVER2002638 | 0 | 0 | 0 | 0 | 0 | 29.377 | 0 |
| LIVER2002842 | 0 | 4.380 | 0 | 0 | 0 | 0 | 6.351 |
| LIVER2005028 | 0 | 0 | 0 | 7.709 | 0 | 0 | 0 |
| LIVER2005789 | 0 | 0 | 0 | 0 | 0 | 0 | 4.992 |
| LIVER2005973 | 0 | 0 | 0 | 10.530 | 0 | 0 | 0 |
| LIVER2006410 | 0 | 0 | 0 | 0 | 0 | 0 | 11.342 |
| LIVER2008465 | 0 | 0 | 0 | 6.752 | 0 | 0 | 7.339 |
| LIVER2008751 | 0 | 0 | 0 | 0 | 0 | 0 | 9.629 |
| MAMGL1000017 | 0 | 0 | 0 | 0 | 1.132 | 0 | 0 |
| MAMGL1000032 | 0 | 0.370 | 0.571 | 0.164 | 0 | 0.581 | 0.179 |
| MAMGL1000035 | 0.201 | 0 | 0 | 0 | 0 | 0 | 0 |
| MAMGL1000083 | 0 | 0 | 0 | 0.296 | 0 | 0 | 0.321 |
| MAMGL1000100 | 0 | 0 | 0 | 0 | 0 | 2.129 | 0 |
| MESAN1000032 | 0 | 0 | 0 | 0 | 0 | 0 | 23.021 |
| MESAN1000050 | 4.816 | 0 | 0 | 0 | 0 | 0 | 3.560 |
| MESAN1000079 | 0 | 0 | 0 | 14.664 | 0 | 0 | 15.939 |
| MESAN1000080 | 0 | 0 | 0 | 0 | 0 | 0 | 1.583 |
| MESAN1000101 | 0 | 0 | 0 | 0 | 0 | 0 | 6.401 |
| MESAN1000126 | 1.786 | 0 | 0 | 3.644 | 0 | 0 | 1.980 |
| MESAN2000092 | 8.849 | 0 | 0 | 0 | 0 | 0 | 9.813 |
| MESAN2000149 | 0 | 0 | 17.405 | 0 | 9.143 | 0 | 5.448 |
| MESAN2000264 | 0 | 4.521 | 0 | 0 | 0 | 0 | 0 |
| MESAN2000267 | 1.638 | 0.939 | 0 | 2.089 | 0 | 0 | 2.725 |
| MESAN2000501 | 0 | 0 | 0 | 0 | 0 | 0 | 3.327 |
| MESAN2000572 | 0 | 2.346 | 0 | 1.043 | 0 | 3.686 | 3.401 |
| MESAN2000608 | 0.786 | 0 | 0 | 0.802 | 2.927 | 0 | 0.872 |
| MESAN2001154 | 1.332 | 0 | 0 | 0 | 9.918 | 0 | 0 |
| MESAN2001627 | 13.080 | 0 | 0 | 0 | 0 | 0 | 0 |
| MESAN2002086 | 0 | 0 | 0 | 0.849 | 0 | 0 | 1.847 |
| MESAN2002122 | 8.870 | 0 | 0 | 9.050 | 0 | 0 | 9.836 |
| MESAN2002186 | 0 | 0 | 0 | 0 | 0 | 7.904 | 0 |
| MESAN2002519 | 0 | 0 | 0 | 4.732 | 0 | 0 | 5.143 |
| MESAN2002724 | 0.922 | 0 | 0 | 1.882 | 0 | 0 | 0 |
| MESAN2002844 | 0 | 0 | 0 | 7.393 | 0 | 0 | 8.036 |
| MESAN2002940 | 3.180 | 0 | 0 | 0 | 0 | 0 | 0 |
| MESAN2003190 | 0 | 0 | 0 | 0 | 0 | 0 | 19.624 |
| MESAN2003444 | 3.725 | 0 | 0 | 3.801 | 0 | 13.432 | 0 |
| MESAN2003622 | 0 | 0 | 0 | 0 | 0 | 0 | 1.999 |
| MESAN2003646 | 0 | 0 | 0 | 0 | 0 | 0 | 23.021 |
| MESAN2003709 | 0 | 0 | 29.383 | 0 | 0 | 0 | 0 |
| MESAN2005284 | 0 | 0 | 0 | 0 | 0 | 0 | 10.755 |
| MESAN2005689 | 0 | 0 | 0 | 3.165 | 0 | 0 | 10.321 |
| MESAN2005766 | 0 | 2.262 | 0 | 0 | 0 | 0 | 5.468 |
| MESAN2005957 | 0 | 0 | 0 | 10.074 | 0 | 0 | 0 |
| MESAN2005958 | 5.386 | 0 | 0 | 2.748 | 10.023 | 0 | 2.986 |
| MESAN2006043 | 0 | 0 | 0 | 8.126 | 0 | 0 | 8.833 |
| MESAN2006401 | 3.618 | 12.451 | 6.409 | 5.537 | 13.467 | 6.523 | 4.012 |
| MESAN2006563 | 0.691 | 0 | 0 | 0 | 2.570 | 0 | 1.532 |
| MESAN2006743 | 0 | 0 | 0 | 0 | 0 | 0 | 8.549 |
| MESAN2006953 | 0 | 0 | 0 | 6.996 | 0 | 0 | 7.604 |
| MESAN2007755 | 0 | 0 | 0 | 9.333 | 0 | 0 | 0 |
| MESAN2008926 | 3.192 | 0 | 0 | 0 | 0 | 0 | 0 |
| MESAN2008936 | 3.978 | 0 | 0 | 4.059 | 0 | 0 | 0 |
| MESAN2009081 | 0 | 10.799 | 0 | 9.604 | 17.519 | 0 | 0 |
| MESAN2009503 | 0 | 38.222 | 0 | 0 | 0 | 0 | 0 |
| MESAN2009580 | 0 | 2.548 | 0 | 1.133 | 4.133 | 0 | 1.232 |
| MESAN2010664 | 0 | 19.910 | 0 | 0 | 0 | 0 | 9.624 |
| MESAN2011632 | 0 | 0 | 0 | 21.577 | 0 | 0 | 0 |
| MESAN2011984 | 0 | 0 | 6.044 | 1.741 | 6.350 | 0 | 1.892 |
| MESAN2012054 | 12.754 | 0 | 0 | 15.615 | 0 | 0 | 0 |
| MESAN2012113 | 0 | 0 | 0 | 6.019 | 0 | 0 | 6.543 |
| MESAN2013283 | 0 | 38.222 | 0 | 0 | 0 | 0 | 0 |
| MESAN2013284 | 2.456 | 0 | 0 | 5.012 | 0 | 0 | 1.362 |
| MESAN2016409 | 0 | 38.222 | 0 | 0 | 0 | 0 | 0 |
| MESAN2017133 | 17.548 | 0 | 0 | 0 | 0 | 0 | 0 |
| MESTC1000042 | 2.245 | 0 | 0 | 0 | 0 | 0 | 0 |
| NB9N41000011 | 2.128 | 0 | 0 | 0 | 0 | 0 | 2.360 |
| NB9N41000047 | 0 | 0 | 0 | 0 | 0 | 0 | 2.915 |
| NB9N41000142 | 0 | 0 | 0 | 0 | 0 | 0 | 2.969 |
| NB9N41000340 | 0 | 0 | 0 | 0 | 0 | 5.229 | 0 |
| NB9N42000281 | 1.912 | 4.387 | 0 | 0 | 0 | 6.894 | 0 |
| NB9N42000342 | 0 | 1.654 | 0 | 0.736 | 0 | 0 | 3.198 |
| NB9N42000430 | 0 | 6.350 | 0 | 0 | 0 | 0 | 0 |
| NB9N42001300 | 5.414 | 0 | 0 | 0 | 0 | 0 | 3.002 |
| NHNPC2000062 | 0 | 0 | 0 | 0 | 0 | 0 | 2.324 |
| NHNPC2000206 | 0 | 1.575 | 2.433 | 0.701 | 0 | 0 | 3.046 |
| NHNPC2001931 | 0.477 | 0 | 0 | 0 | 0 | 0 | 0.529 |
| NOVAR1000015 | 0 | 0 | 10.757 | 0 | 0 | 0 | 0 |
| NOVAR2000038 | 1.242 | 4.275 | 6.602 | 0 | 9.248 | 17.918 | 0 |
| NOVAR2001783 | 4.027 | 0 | 0 | 0 | 0 | 0 | 0 |
| NT2NE1000004 | 0 | 0 | 0 | 0 | 0 | 0 | 9.158 |
| NT2NE1000018 | 0 | 5.705 | 0 | 5.074 | 0 | 0 | 2.758 |
| NT2NE1000073 | 7.963 | 0 | 0 | 0 | 0 | 0 | 0 |
| NT2NE1000163 | 8.911 | 0.929 | 0 | 10.745 | 0 | 0 | 2.246 |
| NT2NE1000181 | 0 | 0 | 0 | 18.624 | 0 | 0 | 0 |
| NT2NE2000056 | 6.285 | 0.901 | 0 | 7.615 | 4.386 | 1.416 | 3.921 |
| NT2NE2000174 | 0 | 0.875 | 1.352 | 0 | 0 | 0 | 0.423 |
| NT2NE2000195 | 0 | 0 | 0 | 1.568 | 5.721 | 0 | 0 |
| NT2NE2000260 | 0 | 0 | 1.284 | 0.246 | 0.450 | 0.871 | 0.804 |
| NT2NE2000327 | 2.516 | 0 | 0 | 0 | 0 | 0 | 2.790 |
| NT2NE2000383 | 0 | 0 | 5.383 | 3.101 | 0 | 5.479 | 6.740 |
| NT2NE2000384 | 6.905 | 0 | 0 | 4.696 | 0 | 0 | 10.210 |
| NT2NE2000386 | 15.452 | 0 | 0 | 0 | 0 | 0 | 0 |
| NT2NE2000392 | 0.893 | 2.048 | 0 | 6.376 | 3.323 | 3.219 | 3.960 |
| NT2NE2000548 | 0 | 0 | 0 | 0 | 0 | 0 | 23.430 |
| NT2NE2000575 | 0.721 | 2.206 | 0.852 | 0.736 | 0 | 1.734 | 2.133 |
| NT2NE2000636 | 7.222 | 0 | 0 | 11.052 | 0 | 0 | 0 |
| NT2NE2000707 | 2.342 | 1.535 | 4.742 | 3.073 | 4.982 | 4.826 | 0.371 |
| NT2NE2000763 | 1.200 | 0 | 4.251 | 2.449 | 0 | 4.327 | 3.993 |
| NT2NE2000809 | 0 | 0 | 0 | 0 | 12.616 | 0 | 0 |
| NT2NE2000909 | 0 | 0 | 0 | 6.679 | 0 | 0 | 0 |
| NT2NE2000963 | 0 | 2.631 | 1.625 | 0 | 0.854 | 1.654 | 1.018 |
| NT2NE2000980 | 0 | 0 | 0 | 0 | 0 | 0 | 16.930 |
| NT2NE2001000 | 0.861 | 1.974 | 6.098 | 1.756 | 6.406 | 0 | 0 |
| NT2NE2001040 | 0 | 0 | 0 | 0 | 0 | 0 | 17.192 |
| NT2NE2001048 | 1.497 | 0 | 0 | 4.581 | 5.571 | 0 | 3.320 |
| NT2NE2001049 | 0 | 0 | 0 | 0 | 0 | 31.686 | 0 |
| NT2NE2001156 | 0 | 0 | 0 | 5.734 | 0 | 0 | 0 |
| NT2NE2001176 | 7.995 | 0 | 4.046 | 2.331 | 0 | 12.355 | 12.533 |
| NT2NE2001180 | 0 | 0 | 49.431 | 0 | 0 | 0 | 0 |
| NT2NE2001337 | 4.544 | 0 | 0 | 0 | 0 | 0 | 0 |
| NT2NE2001364 | 10.691 | 0 | 0 | 0 | 39.792 | 0 | 0 |
| NT2NE2001397 | 0 | 0 | 0 | 0 | 0 | 0 | 14.585 |
| NT2NE2001403 | 10.376 | 0 | 0 | 0 | 19.310 | 0 | 5.753 |
| NT2NE2001435 | 5.316 | 0 | 0 | 11.931 | 0 | 0 | 2.358 |
| NT2NE2001442 | 4.997 | 0 | 0 | 15.295 | 0 | 0 | 11.083 |
| NT2NE2001545 | 12.542 | 0 | 0 | 5.484 | 0 | 0 | 1.987 |
| NT2NE2001598 | 0 | 0 | 0 | 0 | 0 | 45.737 | 0 |
| NT2NE2001648 | 0 | 0 | 30.746 | 0 | 0 | 0 | 0 |
| NT2NE2001655 | 0 | 9.636 | 4.960 | 2.857 | 10.422 | 5.048 | 0 |
| NT2NE2001660 | 7.832 | 0 | 0 | 7.991 | 0 | 0 | 8.686 |
| NT2NE2001793 | 11.409 | 0 | 0 | 7.408 | 0 | 0 | 2.300 |
| NT2NE2002162 | 0 | 0 | 0 | 0 | 45.501 | 0 | 0 |
| NT2NE2003185 | 0 | 17.393 | 0 | 0 | 0 | 0 | 4.204 |
| NT2NE2003280 | 7.277 | 0 | 25.781 | 0 | 0 | 0 | 0 |
| NT2NE2003485 | 2.328 | 0 | 0 | 0 | 0 | 0 | 5.163 |
| NT2NE2003569 | 3.471 | 0 | 0 | 0 | 0 | 0 | 7.698 |
| NT2NE2003887 | 6.241 | 0 | 0 | 0 | 0 | 0 | 0 |
| NT2NE2003921 | 0 | 0 | 0 | 0 | 0 | 0 | 6.765 |
| NT2NE2004255 | 0 | 0 | 0 | 0 | 0 | 0 | 6.605 |
| NT2NE2004519 | 0 | 0 | 0 | 0 | 6.669 | 0 | 3.974 |
| NT2NE2005419 | 11.579 | 0 | 0 | 0 | 0 | 0 | 2.840 |
| NT2NE2005588 | 0 | 38.765 | 0 | 0 | 0 | 0 | 0 |
| NT2NE2005688 | 1.493 | 0 | 0 | 0 | 0 | 0 | 0.828 |
| NT2NE2005876 | 1.475 | 3.384 | 0 | 1.505 | 0 | 0 | 1.636 |
| NT2NE2006075 | 0 | 0 | 0 | 0 | 0 | 28.404 | 0 |
| NT2NE2006478 | 0 | 0 | 0 | 10.962 | 0 | 0 | 11.916 |
| NT2NE2006909 | 0.285 | 1.964 | 0 | 0 | 0 | 0 | 0 |
| NT2NE2008213 | 0 | 19.092 | 0 | 0 | 0 | 30.005 | 0 |
| NT2NE2009295 | 0 | 0 | 0 | 2.035 | 0 | 7.194 | 2.212 |
| NT2NE2009523 | 0 | 0 | 0 | 0 | 0 | 28.018 | 0 |
| NT2NE2010056 | 0 | 0 | 0 | 0 | 0 | 0 | 17.773 |
| NT2NE2010688 | 21.626 | 0 | 0 | 0 | 0 | 0 | 0 |
| NT2NE2010842 | 3.771 | 2.163 | 3.340 | 1.924 | 0 | 0 | 1.046 |
| NT2NE2011119 | 0 | 10.623 | 0 | 0 | 0 | 0 | 0 |
| NT2NE2011221 | 0 | 0 | 0 | 0 | 0 | 0 | 47.862 |
| NT2NE2011485 | 18.947 | 0 | 0 | 0 | 0 | 0 | 0 |
| NT2NE2012243 | 0 | 0 | 0 | 0 | 0 | 0 | 23.430 |
| NT2NE2012928 | 0 | 0 | 0 | 0 | 0 | 31.392 | 0 |
| NT2NE2014104 | 3.357 | 3.851 | 0 | 0 | 0 | 0 | 0 |
| NT2NE2015904 | 0 | 0 | 0 | 21.967 | 0 | 0 | 0 |
| NT2NE2015974 | 0 | 0 | 0 | 0 | 0 | 0 | 8.471 |
| NT2NE2016387 | 21.626 | 0 | 0 | 0 | 0 | 0 | 0 |
| NT2NE2017492 | 0 | 0 | 0 | 0 | 0 | 0 | 18.714 |
| NT2NE2018594 | 0 | 38.765 | 0 | 0 | 0 | 0 | 0 |
| NT2NE2019091 | 5.679 | 0 | 13.413 | 0 | 7.046 | 0 | 0 |
| NT2RI1000016 | 0 | 0 | 0 | 25.779 | 0 | 0 | 0 |
| NT2RI1000048 | 0 | 0 | 0 | 0 | 0 | 0 | 1.883 |
| NT2RI1000127 | 5.445 | 0 | 0 | 0 | 6.755 | 0 | 0 |
| NT2RI2000064 | 0.538 | 0 | 0 | 0.549 | 0 | 0 | 0.597 |
| NT2RI2000107 | 0 | 5.314 | 0 | 0 | 0 | 0 | 0 |
| NT2RI2000116 | 4.071 | 0 | 0 | 3.560 | 0 | 0 | 1.935 |
| NT2RI2000167 | 0 | 0 | 0 | 11.787 | 0 | 0 | 12.812 |
| NT2RI2000255 | 0 | 2.746 | 0 | 0 | 0 | 0 | 1.327 |
| NT2RI2000263 | 3.421 | 0 | 0 | 3.490 | 0 | 0 | 0 |
| NT2RI2000284 | 2.279 | 1.046 | 0 | 0 | 0 | 1.644 | 0 |
| NT2RI2000294 | 1.893 | 0 | 0 | 1.931 | 0 | 0 | 2.099 |
| NT2RI2000344 | 0 | 0 | 0 | 0 | 3.013 | 0 | 1.796 |
| NT2RI2000578 | 0 | 0 | 0 | 0.341 | 0 | 0 | 0 |
| NT2RI2000671 | 0 | 0 | 0 | 0 | 3.915 | 0 | 0 |
| NT2RI2000685 | 0 | 2.273 | 0 | 0 | 3.688 | 0 | 0 |
| NT2RI2000727 | 1.552 | 1.187 | 0 | 1.848 | 1.926 | 1.866 | 0.861 |
| NT2RI2000775 | 0 | 0 | 0 | 0 | 0 | 0 | 18.913 |
| NT2RI2000829 | 3.819 | 0 | 0 | 3.897 | 0 | 0 | 0 |
| NT2RI2000974 | 2.157 | 4.949 | 0 | 2.201 | 0 | 0 | 4.784 |
| NT2RI2001054 | 0.595 | 1.365 | 2.108 | 0 | 2.215 | 0 | 0.660 |
| NT2RI2001230 | 0 | 0 | 0 | 0.787 | 0 | 0 | 0.855 |
| NT2RI2001422 | 0 | 8.529 | 0 | 0 | 0 | 0 | 0 |
| NT2RI2001450 | 3.544 | 12.195 | 6.277 | 10.846 | 0 | 0 | 5.895 |
| NT2RI2001540 | 0 | 0.522 | 0.403 | 0 | 1.271 | 0.616 | 0.063 |
| NT2RI2001621 | 3.061 | 0 | 0 | 0 | 11.393 | 0 | 0 |
| NT2RI2001657 | 0 | 0 | 1.895 | 0 | 0 | 0 | 1.780 |
| NT2RI2001726 | 10.559 | 0 | 0 | 10.773 | 0 | 0 | 0 |
| NT2RI2001731 | 0 | 0 | 0 | 1.932 | 7.049 | 0 | 0 |
| NT2RI2001846 | 9.856 | 0 | 0 | 0 | 0 | 0 | 0 |
| NT2RI2001859 | 2.911 | 0 | 0 | 1.980 | 0 | 0 | 1.076 |
| NT2RI2001910 | 0 | 0 | 0 | 0 | 0 | 26.931 | 0 |
| NT2RI2001952 | 0 | 0 | 0 | 0 | 0 | 0 | 23.231 |
| NT2RI2002091 | 0.040 | 0.369 | 0.475 | 0.055 | 0.150 | 0.097 | 0.476 |
| NT2RI2002152 | 0 | 0 | 0 | 1.283 | 4.681 | 0 | 0 |
| NT2RI2002243 | 0 | 0 | 0 | 0 | 0 | 0 | 3.648 |
| NT2RI2002266 | 0 | 0 | 0 | 2.557 | 0 | 0 | 0 |
| NT2RI2002316 | 0 | 0 | 2.840 | 0.818 | 0 | 0 | 0.889 |
| NT2RI2002359 | 8.230 | 0 | 0 | 0 | 0 | 0 | 0 |
| NT2RI2002391 | 0 | 1.860 | 0 | 0 | 0 | 0 | 1.799 |
| NT2RI2002530 | 0 | 13.848 | 0 | 0 | 0 | 0 | 0 |
| NT2RI2002540 | 6.915 | 7.932 | 0 | 5.291 | 6.434 | 24.933 | 1.917 |
| NT2RI2002549 | 0 | 0 | 0 | 0 | 0 | 0 | 7.641 |
| NT2RI2002592 | 0 | 0 | 0 | 0 | 0 | 66.490 | 0 |
| NT2RI2002602 | 0 | 0 | 0 | 0 | 29.147 | 0 | 0 |
| NT2RI2002654 | 0.587 | 0 | 0 | 1.199 | 2.186 | 2.118 | 1.303 |
| NT2RI2002699 | 0 | 26.231 | 0 | 0 | 0 | 0 | 0 |
| NT2RI2002819 | 7.446 | 0 | 13.189 | 0 | 13.857 | 0 | 4.129 |
| NT2RI2002822 | 5.017 | 0 | 0 | 10.238 | 0 | 0 | 22.256 |
| NT2RI2002847 | 0 | 0 | 0 | 1.281 | 0 | 0 | 2.784 |
| NT2RI2002926 | 0 | 0 | 0 | 0 | 0 | 28.456 | 0 |
| NT2RI2002958 | 3.851 | 0 | 3.411 | 2.947 | 3.583 | 3.471 | 10.677 |
| NT2RI2003011 | 0 | 0 | 0 | 0 | 0 | 0 | 3.829 |
| NT2RI2003051 | 9.214 | 0 | 3.627 | 4.178 | 0 | 0 | 3.406 |
| NT2RI2003205 | 0 | 0 | 0 | 3.284 | 0 | 0 | 0 |
| NT2RI2003222 | 0 | 0 | 0 | 0 | 0 | 0 | 28.191 |
| NT2RI2003301 | 17.027 | 0 | 0 | 0 | 0 | 0 | 0 |
| NT2RI2003304 | 0.525 | 0 | 0 | 1.606 | 0.977 | 0 | 0.873 |
| NT2RI2003317 | 9.458 | 3.616 | 0 | 3.216 | 0 | 0 | 1.748 |
| NT2RI2003338 | 6.075 | 0 | 0 | 3.099 | 0 | 0 | 3.369 |
| NT2RI2003344 | 11.684 | 0 | 0 | 19.868 | 0 | 0 | 8.638 |
| NT2RI2003361 | 4.939 | 22.661 | 0 | 0 | 0 | 17.808 | 0 |
| NT2RI2003420 | 9.240 | 0 | 0 | 4.713 | 0 | 0 | 1.708 |
| NT2RI2003481 | 3.862 | 0 | 0 | 5.910 | 0 | 0 | 0 |
| NT2RI2003556 | 0 | 0 | 0 | 0 | 0 | 0 | 0.422 |
| NT2RI2003655 | 0 | 44.817 | 0 | 0 | 0 | 0 | 0 |
| NT2RI2003695 | 1.575 | 5.420 | 0 | 0 | 0 | 0 | 2.620 |
| NT2RI2003738 | 0.175 | 0 | 0 | 0 | 0.651 | 0.631 | 0 |
| NT2RI2003751 | 1.385 | 3.178 | 0 | 0 | 5.156 | 0 | 0 |
| NT2RI2003921 | 0.474 | 0 | 0 | 0.484 | 0 | 0 | 0 |
| NT2RI2004078 | 35.496 | 0 | 0 | 0 | 0 | 0 | 0 |
| NT2RI2004188 | 0 | 0 | 0 | 0 | 0 | 10.353 | 0 |
| NT2RI2004230 | 0 | 7.897 | 0 | 0 | 0 | 0 | 0 |
| NT2RI2004304 | 8.329 | 0 | 0 | 4.249 | 0 | 0 | 9.236 |
| NT2RI2004398 | 4.982 | 0 | 0 | 0 | 0 | 0 | 5.525 |
| NT2RI2004410 | 4.988 | 0 | 0 | 7.634 | 0 | 8.993 | 2.766 |
| NT2RI2004535 | 7.657 | 0 | 0 | 0 | 0 | 0 | 10.614 |
| NT2RI2004606 | 35.496 | 0 | 0 | 0 | 0 | 0 | 0 |
| NT2RI2004608 | 0 | 8.488 | 0 | 0 | 0 | 0 | 0 |
| NT2RI2004783 | 21.577 | 0 | 0 | 0 | 0 | 0 | 0 |
| NT2RI2004884 | 0 | 0 | 0 | 0.788 | 2.876 | 0 | 0 |
| NT2RI2005116 | 0 | 0 | 4.528 | 0 | 0 | 0 | 1.418 |
| NT2RI2005150 | 2.756 | 0 | 0 | 4.218 | 2.565 | 0 | 0 |
| NT2RI2005239 | 0 | 9.048 | 0 | 12.070 | 0 | 0 | 4.373 |
| NT2RI2005335 | 3.871 | 0 | 0 | 1.975 | 0 | 0 | 3.005 |
| NT2RI2005353 | 0 | 0 | 0 | 5.876 | 0 | 4.153 | 1.277 |
| NT2RI2005368 | 0 | 0 | 7.158 | 4.123 | 0 | 0 | 0 |
| NT2RI2005382 | 0 | 0 | 0 | 20.181 | 0 | 0 | 43.872 |
| NT2RI2005405 | 0.561 | 0 | 0 | 0.573 | 3.134 | 0 | 1.245 |
| NT2RI2005564 | 8.908 | 0 | 0 | 13.633 | 0 | 0 | 0 |
| NT2RI2005579 | 0 | 0 | 0 | 19.149 | 0 | 0 | 0 |
| NT2RI2005713 | 2.958 | 6.786 | 0 | 1.509 | 0 | 0 | 3.280 |
| NT2RI2005723 | 1.505 | 3.454 | 0 | 1.024 | 1.868 | 3.619 | 1.113 |
| NT2RI2005811 | 0 | 0 | 5.427 | 0 | 0 | 0 | 0 |
| NT2RI2005814 | 0 | 0 | 0 | 0 | 0 | 4.696 | 0 |
| NT2RI2005816 | 0 | 0 | 0 | 0 | 0 | 0 | 7.088 |
| NT2RI2005818 | 0 | 0 | 0 | 2.598 | 0 | 0 | 0 |
| NT2RI2005851 | 3.727 | 0 | 0 | 3.802 | 0 | 0 | 0 |
| NT2RI2006071 | 16.937 | 0 | 0 | 0 | 0 | 0 | 0 |
| NT2RI2006072 | 0.765 | 3.511 | 0 | 0.781 | 0 | 2.759 | 0.848 |
| NT2RI2006271 | 2.793 | 0 | 0 | 0 | 0 | 0 | 0 |
| NT2RI2006412 | 6.558 | 9.403 | 5.808 | 4.181 | 0 | 2.956 | 0 |
| NT2RI2006553 | 0 | 8.485 | 0 | 0 | 0 | 0 | 0 |
| NT2RI2006640 | 6.285 | 0 | 0 | 12.824 | 0 | 0 | 6.970 |
| NT2RI2006647 | 0 | 10.520 | 0 | 0 | 0 | 0 | 0 |
| NT2RI2006703 | 0 | 0 | 0 | 0 | 0 | 0 | 17.973 |
| NT2RI2006735 | 15.739 | 0 | 0 | 16.057 | 0 | 0 | 0 |
| NT2RI2006825 | 2.742 | 0 | 0 | 0 | 0 | 0 | 3.040 |
| NT2RI2006856 | 0 | 1.075 | 0 | 0.478 | 3.487 | 0 | 0.519 |
| NT2RI2006943 | 0 | 2.790 | 0 | 1.241 | 0 | 13.156 | 0 |
| NT2RI2007054 | 1.298 | 0 | 0 | 0 | 0 | 0 | 1.440 |
| NT2RI2007084 | 0 | 0 | 3.854 | 0 | 0 | 0 | 3.619 |
| NT2RI2007096 | 10.368 | 0 | 0 | 10.578 | 0 | 0 | 5.749 |
| NT2RI2007133 | 0.392 | 0 | 0 | 0 | 0 | 0 | 0 |
| NT2RI2007148 | 0 | 0 | 0 | 0 | 0 | 0 | 1.047 |
| NT2RI2007277 | 0 | 0 | 0 | 3.001 | 0 | 0 | 5.436 |
| NT2RI2007377 | 3.514 | 5.374 | 0 | 1.195 | 0 | 4.223 | 0 |
| NT2RI2007384 | 0 | 2.981 | 0 | 0 | 1.612 | 0 | 0 |
| NT2RI2007445 | 0 | 0 | 0 | 0 | 6.674 | 6.465 | 3.977 |
| NT2RI2007468 | 0 | 0 | 0 | 0 | 0 | 0 | 4.634 |
| NT2RI2007498 | 0 | 0 | 0 | 0 | 0 | 0 | 0.452 |
| NT2RI2007572 | 1.494 | 3.428 | 0 | 1.525 | 0 | 2.694 | 0.829 |
| NT2RI2007589 | 3.204 | 4.900 | 3.783 | 0 | 7.950 | 0 | 0 |
| NT2RI2007593 | 0 | 0 | 0 | 0 | 0 | 0 | 3.654 |
| NT2RI2007884 | 0 | 5.824 | 8.994 | 0 | 0 | 9.154 | 5.631 |
| NT2RI2007987 | 1.801 | 0 | 0 | 1.837 | 0 | 0 | 1.997 |
| NT2RI2008045 | 2.329 | 0 | 2.751 | 0 | 0 | 0 | 0 |
| NT2RI2008141 | 0 | 0 | 0 | 0 | 0 | 0 | 2.371 |
| NT2RI2008171 | 0 | 10.187 | 0 | 0 | 0 | 0 | 0 |
| NT2RI2008336 | 0 | 0.919 | 0 | 1.226 | 0 | 0 | 0.889 |
| NT2RI2008396 | 0.441 | 1.011 | 0 | 0.450 | 0 | 0 | 2.444 |
| NT2RI2008481 | 0 | 0 | 0 | 0.755 | 0 | 0 | 1.640 |
| NT2RI2008526 | 0 | 0 | 0 | 0.782 | 0 | 0 | 0 |
| NT2RI2008566 | 0 | 0 | 0 | 9.596 | 0 | 0 | 20.861 |
| NT2RI2008622 | 0 | 0 | 0 | 0 | 0 | 0 | 6.999 |
| NT2RI2008656 | 2.716 | 0 | 0 | 0 | 0 | 0 | 3.012 |
| NT2RI2008724 | 1.836 | 0 | 0 | 1.874 | 0 | 0 | 4.073 |
| NT2RI2008749 | 0.565 | 1.297 | 0 | 0 | 2.104 | 0 | 1.254 |
| NT2RI2008791 | 0 | 5.353 | 0 | 1.190 | 0 | 0 | 1.294 |
| NT2RI2008812 | 0 | 0 | 0 | 0 | 0 | 26.104 | 0 |
| NT2RI2008942 | 0 | 0 | 0 | 3.210 | 0 | 0 | 0 |
| NT2RI2009037 | 0.843 | 0 | 0 | 1.720 | 0 | 0 | 0 |
| NT2RI2009065 | 1.401 | 0 | 1.655 | 0 | 1.738 | 0 | 0.518 |
| NT2RI2009066 | 0 | 6.288 | 0 | 8.389 | 0 | 0 | 0 |
| NT2RI2009173 | 0 | 0 | 0 | 1.138 | 0 | 0 | 1.237 |
| NT2RI2009194 | 1.965 | 0 | 0 | 1.003 | 0 | 0 | 0 |
| NT2RI2009233 | 6.767 | 5.175 | 7.991 | 0 | 0 | 8.133 | 2.501 |
| NT2RI2009239 | 1.398 | 0 | 0 | 0 | 0 | 0 | 0 |
| NT2RI2009269 | 1.976 | 0 | 0 | 1.008 | 0 | 7.126 | 1.096 |
| NT2RI2009281 | 1.407 | 6.458 | 0 | 1.436 | 0 | 0 | 0 |
| NT2RI2009402 | 6.273 | 7.196 | 0 | 3.200 | 11.674 | 0 | 3.478 |
| NT2RI2009406 | 0 | 0 | 0 | 2.870 | 0 | 10.144 | 0 |
| NT2RI2009583 | 0.224 | 1.540 | 0.793 | 0.228 | 1.665 | 1.613 | 1.489 |
| NT2RI2010795 | 0 | 0 | 0 | 16.827 | 0 | 0 | 36.581 |
| NT2RI2011450 | 25.398 | 0 | 0 | 0 | 0 | 0 | 0 |
| NT2RI2012659 | 13.562 | 0 | 0 | 0 | 0 | 0 | 0 |
| NT2RI2012726 | 23.195 | 0 | 0 | 0 | 0 | 0 | 10.289 |
| NT2RI2013345 | 9.458 | 0 | 0 | 9.649 | 0 | 0 | 0 |
| NT2RI2014733 | 24.938 | 0 | 0 | 0 | 0 | 0 | 0 |
| NT2RI2015342 | 1.504 | 0 | 0 | 1.535 | 11.198 | 0 | 5.005 |
| NT2RI2015533 | 6.216 | 7.131 | 0 | 1.586 | 0 | 5.604 | 12.064 |
| NT2RI2018311 | 0 | 0 | 0 | 21.918 | 0 | 0 | 0 |
| NT2RI2021625 | 0 | 0 | 0 | 19.442 | 0 | 0 | 0 |
| NT2RI2024313 | 0 | 0 | 0 | 14.948 | 0 | 0 | 0 |
| NT2RI2025075 | 0 | 0 | 0 | 17.297 | 0 | 0 | 18.801 |
| NT2RI2025255 | 0 | 0 | 0 | 0 | 0 | 0 | 37.898 |
| NT2RI3001445 | 0 | 0 | 0 | 19.385 | 0 | 22.837 | 21.071 |
| NT2RI3001515 | 0 | 0 | 0 | 11.050 | 0 | 0 | 0 |
| NT2RI3002842 | 0 | 0 | 0 | 0 | 0 | 0 | 20.781 |
| NT2RI3003738 | 18.137 | 0 | 0 | 0 | 0 | 0 | 0 |
| NT2RI3003925 | 0 | 0 | 0 | 35.956 | 0 | 0 | 0 |
| NT2RI3004510 | 0 | 0 | 0 | 28.761 | 0 | 0 | 0 |
| NT2RI3005403 | 0 | 0 | 0 | 0 | 0 | 0 | 37.898 |
| NT2RI3005724 | 0 | 0 | 0 | 12.093 | 0 | 0 | 0 |
| NT2RI3005923 | 19.493 | 0 | 0 | 0 | 0 | 0 | 0 |
| NT2RI3006284 | 4.138 | 0 | 0 | 0 | 0 | 0 | 0 |
| NT2RI3006673 | 19.959 | 0 | 0 | 10.182 | 0 | 0 | 0 |
| NT2RI3007291 | 0 | 0 | 0 | 12.770 | 0 | 0 | 0 |
| NT2RI3007543 | 1.697 | 0 | 0 | 0.866 | 0 | 3.059 | 0 |
| NT2RI3008055 | 44.179 | 0 | 0 | 0 | 0 | 0 | 0 |
| NT2RI3009524 | 35.496 | 0 | 0 | 0 | 0 | 0 | 0 |
| NT2RP6000017 | 2.992 | 0 | 0 | 1.526 | 0 | 0 | 0 |
| NT2RP6000039 | 8.966 | 0 | 0 | 3.659 | 0 | 0 | 0 |
| NT2RP6000043 | 3.798 | 0 | 13.455 | 3.875 | 0 | 13.695 | 4.212 |
| NT2RP6000072 | 0 | 0 | 5.724 | 0 | 0 | 0 | 0 |
| NT2RP6000077 | 0 | 0 | 0 | 0 | 0 | 0 | 7.128 |
| NT2RP6000078 | 1.079 | 0 | 0 | 0.734 | 0 | 0 | 0.399 |
| NT2RP6000085 | 4.625 | 0 | 0 | 0 | 0 | 0 | 5.129 |
| NT2RP6000109 | 0 | 0 | 0 | 0 | 0 | 0 | 2.118 |
| NT2RP6000110 | 2.738 | 0 | 0 | 0 | 0 | 0 | 18.220 |
| NT2RP6000123 | 0 | 2.694 | 1.387 | 0.399 | 0 | 0 | 0 |
| NT2RP7000069 | 10.105 | 0 | 0 | 3.437 | 0 | 0 | 0 |
| NT2RP7000076 | 0.609 | 1.398 | 4.318 | 0 | 0 | 0 | 1.352 |
| NT2RP7000173 | 2.224 | 0 | 1.969 | 2.836 | 0 | 0 | 0.617 |
| NT2RP7000238 | 0 | 0 | 0 | 1.321 | 0 | 0 | 1.436 |
| NT2RP7000259 | 13.372 | 30.679 | 0 | 0 | 0 | 0 | 0 |
| NT2RP7000271 | 6.066 | 0 | 10.744 | 6.188 | 0 | 10.936 | 0 |
| NT2RP7000477 | 7.914 | 4.539 | 0 | 6.055 | 0 | 0 | 4.388 |
| NT2RP7000586 | 10.987 | 0 | 0 | 11.209 | 0 | 0 | 0 |
| NT2RP7000600 | 6.648 | 0 | 0 | 13.565 | 0 | 0 | 7.372 |
| NT2RP7000624 | 8.726 | 0 | 0 | 0 | 0 | 15.732 | 0 |
| NT2RP7000906 | 10.436 | 0 | 0 | 2.662 | 0 | 4.704 | 7.233 |
| NT2RP7001080 | 3.347 | 0 | 0 | 0 | 0 | 0 | 0 |
| NT2RP7001166 | 2.992 | 1.716 | 0 | 1.526 | 0 | 0 | 3.318 |
| NT2RP7001231 | 4.130 | 0 | 0 | 4.213 | 0 | 0 | 13.739 |
| NT2RP7001283 | 0 | 0 | 1.994 | 1.149 | 0 | 4.059 | 0.624 |
| NT2RP7001306 | 0 | 0 | 25.768 | 0 | 0 | 0 | 0 |
| NT2RP7001335 | 0 | 19.172 | 14.802 | 10.657 | 7.776 | 0 | 6.950 |
| NT2RP7001962 | 5.313 | 0 | 0 | 0 | 0 | 0 | 2.946 |
| NT2RP7002064 | 22.128 | 0 | 0 | 0 | 0 | 0 | 24.539 |
| NT2RP7002376 | 0 | 0 | 0 | 0 | 0 | 0 | 13.455 |
| NT2RP7002379 | 2.014 | 0 | 0 | 2.054 | 1.874 | 0 | 3.908 |
| NT2RP7002449 | 6.061 | 0 | 0 | 6.183 | 0 | 0 | 4.481 |
| NT2RP7002450 | 0 | 0 | 0 | 5.446 | 0 | 0 | 0 |
| NT2RP7002478 | 0 | 0 | 0 | 29.741 | 0 | 0 | 0 |
| NT2RP7002594 | 0 | 0 | 0 | 27.872 | 0 | 0 | 0 |
| NT2RP7002738 | 5.756 | 0 | 0 | 5.873 | 0 | 0 | 6.384 |
| NT2RP7002802 | 0 | 0 | 0 | 3.728 | 0 | 0 | 0 |
| NT2RP7002875 | 9.228 | 0 | 0 | 0.941 | 3.434 | 0 | 6.140 |
| NT2RP7002906 | 5.607 | 6.432 | 0 | 5.721 | 10.435 | 0 | 6.218 |
| NT2RP7003203 | 5.541 | 0 | 0 | 3.769 | 0 | 6.660 | 4.097 |
| NT2RP7003319 | 0 | 2.093 | 3.232 | 4.654 | 0 | 0 | 1.012 |
| NT2RP7003629 | 0 | 0 | 0 | 23.088 | 0 | 0 | 0 |
| NT2RP7003632 | 0 | 0 | 0 | 3.323 | 0 | 0 | 0 |
| NT2RP7003647 | 4.708 | 0 | 4.169 | 15.609 | 0 | 0 | 2.610 |
| NT2RP7003680 | 22.379 | 0 | 0 | 0 | 0 | 0 | 0 |
| NT2RP7003688 | 3.692 | 0 | 0 | 0 | 0 | 0 | 4.913 |
| NT2RP7003960 | 0 | 0 | 0 | 1.690 | 0 | 0 | 1.837 |
| NT2RP7004123 | 0 | 4.403 | 0 | 0 | 0 | 0 | 2.128 |
| NT2RP7004173 | 2.291 | 3.504 | 0 | 1.558 | 0 | 5.507 | 0.847 |
| NT2RP7004196 | 2.620 | 9.018 | 4.641 | 2.673 | 0 | 0 | 0 |
| NT2RP7004204 | 0 | 3.912 | 0 | 5.219 | 6.347 | 0 | 5.673 |
| NT2RP7004233 | 0 | 27.433 | 0 | 0 | 0 | 0 | 13.260 |
| NT2RP7004348 | 0 | 0 | 8.006 | 0 | 0 | 0 | 0 |
| NT2RP7004352 | 3.397 | 0 | 0 | 0 | 0 | 0 | 7.534 |
| NT2RP7004373 | 0 | 0 | 0 | 3.602 | 4.381 | 0 | 5.221 |
| NT2RP7004428 | 16.862 | 0 | 0 | 6.881 | 0 | 0 | 0 |
| NT2RP7004481 | 5.378 | 0 | 0 | 5.487 | 0 | 0 | 0 |
| NT2RP7004559 | 0 | 0 | 0 | 0 | 0 | 0 | 9.452 |
| NT2RP7004728 | 4.715 | 0 | 0 | 4.811 | 0 | 5.667 | 1.743 |
| NT2RP7004751 | 0 | 0 | 0 | 0 | 0 | 1.688 | 0 |
| NT2RP7004790 | 15.609 | 0 | 0 | 0 | 0 | 0 | 8.655 |
| NT2RP7004884 | 13.727 | 0 | 0 | 5.602 | 0 | 0 | 0 |
| NT2RP7004911 | 5.288 | 0 | 0 | 7.193 | 0 | 0 | 5.864 |
| NT2RP7004915 | 7.424 | 0 | 0 | 7.574 | 0 | 0 | 0 |
| NT2RP7004925 | 5.051 | 0 | 0 | 2.576 | 0 | 0 | 0 |
| NT2RP7004946 | 6.026 | 1.975 | 0 | 2.635 | 0 | 0 | 0.955 |
| NT2RP7005513 | 0 | 0 | 0 | 1.724 | 0 | 0 | 0 |
| NT2RP7005529 | 14.766 | 0 | 0 | 15.065 | 0 | 0 | 0 |
| NT2RP7005675 | 11.633 | 0 | 0 | 8.901 | 0 | 0 | 3.225 |
| NT2RP7006033 | 0 | 17.637 | 0 | 0 | 0 | 0 | 17.050 |
| NT2RP7006075 | 0 | 0 | 0 | 0 | 0 | 0 | 5.811 |
| NT2RP7006141 | 0 | 0 | 0 | 0.753 | 0 | 0 | 4.095 |
| NT2RP7006188 | 5.207 | 0 | 0 | 0 | 0 | 0 | 5.774 |
| NT2RP7006223 | 4.225 | 0 | 0 | 8.622 | 15.726 | 0 | 0 |
| NT2RP7006263 | 0 | 0 | 0 | 0 | 0 | 0 | 4.465 |
| NT2RP7006395 | 0 | 0 | 0 | 17.468 | 0 | 0 | 0 |
| NT2RP7006490 | 2.146 | 0 | 0 | 0 | 0 | 7.740 | 2.380 |
| NT2RP7006539 | 0 | 13.349 | 0 | 0 | 10.828 | 0 | 0 |
| NT2RP7006619 | 0 | 0 | 7.819 | 2.252 | 0 | 7.959 | 4.895 |
| NT2RP7006701 | 0 | 0 | 0 | 5.129 | 0 | 0 | 0 |
| NT2RP7006717 | 1.974 | 0 | 0 | 0.671 | 2.449 | 0 | 0.730 |
| NT2RP7006886 | 0 | 0 | 0 | 9.704 | 0 | 0 | 0 |
| NT2RP7006986 | 0 | 0 | 0 | 11.498 | 0 | 0 | 0 |
| NT2RP7007154 | 0 | 1.308 | 0 | 1.164 | 2.122 | 2.056 | 0.632 |
| NT2RP7007177 | 0 | 0 | 0 | 1.389 | 0 | 0 | 0 |
| NT2RP7007252 | 6.867 | 0 | 0 | 7.006 | 0 | 0 | 0 |
| NT2RP7007381 | 15.682 | 0 | 0 | 0 | 0 | 0 | 0 |
| NT2RP7007387 | 6.158 | 4.709 | 0 | 2.094 | 0 | 0 | 2.276 |
| NT2RP7007530 | 0 | 0 | 0 | 2.237 | 0 | 0 | 0 |
| NT2RP7007537 | 1.230 | 0 | 0 | 2.509 | 0 | 0 | 2.727 |
| NT2RP7007610 | 5.788 | 6.640 | 0 | 8.858 | 0 | 0 | 3.209 |
| NT2RP7007617 | 0 | 0 | 0 | 16.050 | 0 | 0 | 17.446 |
| NT2RP7007842 | 0 | 0 | 0 | 1.957 | 7.139 | 0 | 2.127 |
| NT2RP7007975 | 2.729 | 0 | 0 | 1.392 | 0 | 0 | 0 |
| NT2RP7008161 | 0 | 0 | 0 | 6.405 | 0 | 0 | 0 |
| NT2RP7008167 | 2.723 | 0 | 0 | 0.926 | 0 | 0 | 2.013 |
| NT2RP7008190 | 0 | 0 | 0 | 0 | 0 | 0 | 1.471 |
| NT2RP7008315 | 0 | 0 | 0 | 7.656 | 0 | 0 | 0 |
| NT2RP7008396 | 0 | 0 | 0 | 0 | 0 | 0 | 13.651 |
| NT2RP7008487 | 0 | 0 | 0 | 15.611 | 0 | 0 | 0 |
| NT2RP7008543 | 0 | 0 | 0 | 0 | 0 | 0 | 3.051 |
| NT2RP7008714 | 0 | 0 | 0 | 0 | 0 | 0 | 4.301 |
| NT2RP7009012 | 11.338 | 0 | 0 | 11.567 | 0 | 0 | 0 |
| NT2RP7009087 | 2.429 | 0 | 0 | 0 | 0 | 0 | 0 |
| NT2RP7009108 | 4.444 | 8.156 | 0 | 2.720 | 6.616 | 9.614 | 2.957 |
| NT2RP7009147 | 14.337 | 0 | 5.643 | 1.625 | 0 | 0 | 3.533 |
| NT2RP7009149 | 2.393 | 0 | 0 | 6.102 | 0 | 4.313 | 1.327 |
| NT2RP7009225 | 2.813 | 12.906 | 19.929 | 8.609 | 0 | 0 | 0 |
| NT2RP7009259 | 6.180 | 0 | 0 | 5.159 | 0 | 0 | 2.492 |
| NT2RP7009373 | 8.034 | 0 | 0 | 4.098 | 0 | 0 | 17.820 |
| NT2RP7009394 | 13.301 | 0 | 8.567 | 2.467 | 4.501 | 0 | 8.046 |
| NT2RP7009481 | 0 | 0 | 0 | 4.178 | 0 | 0 | 0 |
| NT2RP7009482 | 5.098 | 0 | 0 | 6.619 | 0 | 0 | 3.598 |
| NT2RP7009498 | 0 | 0 | 0 | 0 | 0 | 0 | 2.235 |
| NT2RP7009507 | 29.324 | 0 | 0 | 0 | 0 | 0 | 0 |
| NT2RP7010612 | 0 | 0 | 0 | 22.069 | 0 | 0 | 0 |
| NT2RP7012776 | 0 | 0 | 0 | 0 | 0 | 0 | 9.320 |
| NT2RP7013573 | 0 | 0 | 25.527 | 7.352 | 0 | 0 | 7.991 |
| NT2RP7013764 | 0 | 0 | 0 | 11.583 | 0 | 0 | 0 |
| NT2RP7014005 | 5.818 | 0 | 0 | 0 | 0 | 0 | 0 |
| NT2RP7014910 | 5.978 | 0 | 0 | 6.099 | 0 | 0 | 3.978 |
| NT2RP7015503 | 0 | 0 | 0 | 3.231 | 0 | 11.418 | 0 |
| NT2RP7016574 | 0 | 0 | 0 | 0 | 0 | 0 | 24.498 |
| NT2RP7017474 | 18.823 | 0 | 0 | 0 | 0 | 0 | 0 |
| NT2RP7019135 | 29.324 | 0 | 0 | 0 | 0 | 0 | 0 |
| NT2RP7019401 | 0 | 0 | 0 | 10.593 | 0 | 0 | 0 |
| NT2RP7019543 | 0 | 0 | 0 | 10.792 | 0 | 0 | 0 |
| NT2RP7019835 | 1.042 | 0 | 0 | 0 | 0 | 3.758 | 0 |
| NT2RP8001605 | 29.324 | 0 | 0 | 0 | 0 | 0 | 0 |
| NT2RP8007715 | 25.129 | 0 | 0 | 0 | 0 | 0 | 0 |
| NT2RP8007920 | 1.978 | 0 | 0 | 0 | 7.363 | 0 | 0 |
| NTONG1000033 | 4.901 | 0 | 0 | 7.500 | 0 | 0 | 2.717 |
| NTONG1000098 | 3.823 | 0 | 0 | 3.900 | 0 | 0 | 4.239 |
| NTONG1000182 | 1.238 | 0 | 8.771 | 3.789 | 4.608 | 4.464 | 1.373 |
| NTONG1000264 | 3.899 | 0 | 0 | 4.972 | 0 | 0 | 2.162 |
| NTONG2000107 | 10.664 | 0 | 0 | 0 | 0 | 0 | 0 |
| NTONG2000265 | 7.672 | 1.956 | 3.020 | 13.916 | 0 | 0 | 2.836 |
| NTONG2000876 | 0 | 0 | 4.933 | 0 | 0 | 5.021 | 0 |
| NTONG2000878 | 0 | 0 | 4.183 | 1.205 | 0 | 0 | 2.619 |
| NTONG2000977 | 0 | 2.255 | 3.482 | 0 | 0 | 0 | 0 |
| NTONG2001137 | 1.144 | 2.626 | 0 | 0 | 0 | 0 | 0 |
| NTONG2001428 | 0 | 0 | 0 | 0 | 0 | 0 | 11.690 |
| NTONG2001550 | 3.481 | 0 | 0 | 3.551 | 0 | 0 | 0 |
| NTONG2001567 | 0 | 0 | 0 | 0 | 0 | 0 | 0.340 |
| NTONG2001612 | 1.123 | 2.575 | 3.977 | 0 | 0 | 8.095 | 0 |
| NTONG2001762 | 1.803 | 0 | 6.388 | 9.199 | 6.712 | 0 | 2.000 |
| NTONG2002807 | 0 | 0 | 0 | 0 | 0 | 0 | 13.079 |
| NTONG2002985 | 11.947 | 0 | 0 | 0 | 0 | 0 | 0 |
| NTONG2003454 | 2.981 | 0 | 0 | 18.249 | 0 | 0 | 0 |
| NTONG2003515 | 0 | 0 | 0 | 0 | 0 | 0 | 10.852 |
| NTONG2004095 | 0 | 0 | 0 | 9.367 | 0 | 0 | 0 |
| NTONG2004614 | 0 | 0 | 0 | 0 | 0 | 0 | 6.567 |
| NTONG2005373 | 0 | 0 | 0 | 0 | 0 | 0 | 6.535 |
| NTONG2005391 | 4.662 | 0 | 0 | 14.270 | 0 | 0 | 2.585 |
| NTONG2005520 | 2.663 | 0 | 4.716 | 1.358 | 0 | 0 | 2.953 |
| NTONG2005801 | 14.252 | 0 | 0 | 1.818 | 0 | 0 | 3.951 |
| NTONG2005897 | 0 | 0 | 0 | 7.003 | 0 | 0 | 0 |
| NTONG2005969 | 0 | 14.740 | 0 | 0 | 0 | 0 | 3.562 |
| NTONG2007028 | 11.947 | 0 | 0 | 0 | 0 | 0 | 0 |
| NTONG2007658 | 0 | 0 | 0 | 0 | 0 | 7.216 | 0 |
| NTONG2008088 | 0 | 0 | 0 | 0 | 0 | 0 | 11.332 |
| NTONG2008143 | 10.865 | 0 | 0 | 0 | 0 | 0 | 0 |
| NTONG2008483 | 2.397 | 1.833 | 5.661 | 0.815 | 0 | 5.761 | 1.772 |
| NTONG2008944 | 5.840 | 0 | 0 | 2.979 | 0 | 0 | 3.238 |
| NTONG2009576 | 7.105 | 0 | 0 | 0 | 0 | 0 | 0 |
| OCBBF1000030 | 0 | 0 | 0 | 45.093 | 0 | 0 | 0 |
| OCBBF1000049 | 9.939 | 0 | 0 | 5.070 | 0 | 0 | 0 |
| OCBBF1000080 | 0 | 0 | 0 | 19.507 | 0 | 0 | 0 |
| OCBBF1000086 | 2.296 | 3.512 | 5.423 | 1.562 | 0 | 0 | 0.849 |
| OCBBF1000114 | 0 | 0 | 9.031 | 2.601 | 0 | 0 | 0 |
| OCBBF1000145 | 0 | 0 | 0 | 0 | 0 | 0 | 47.165 |
| OCBBF1000146 | 0.863 | 1.979 | 0 | 0 | 0 | 0 | 1.913 |
| OCBBF1000185 | 4.210 | 0 | 0 | 0 | 0 | 0 | 0 |
| OCBBF1000188 | 1.041 | 0 | 7.378 | 3.187 | 3.876 | 0 | 0 |
| OCBBF1000315 | 0 | 0 | 0 | 8.926 | 0 | 0 | 0 |
| OCBBF2000015 | 9.494 | 0 | 0 | 7.264 | 0 | 0 | 4.387 |
| OCBBF2000126 | 3.887 | 8.917 | 13.769 | 0 | 14.466 | 14.014 | 0 |
| OCBBF2000178 | 4.361 | 0 | 0 | 3.337 | 4.058 | 7.862 | 3.627 |
| OCBBF2000231 | 3.917 | 17.974 | 0 | 7.993 | 0 | 0 | 13.032 |
| OCBBF2000277 | 7.347 | 0 | 0 | 0 | 0 | 0 | 16.294 |
| OCBBF2000522 | 0 | 0 | 0 | 0 | 0 | 0 | 2.804 |
| OCBBF2000712 | 0 | 0 | 0 | 0.964 | 0 | 0 | 1.048 |
| OCBBF2000824 | 0 | 0 | 0 | 6.131 | 0 | 0 | 6.664 |
| OCBBF2000831 | 0 | 0 | 0 | 9.824 | 0 | 0 | 0 |
| OCBBF2000846 | 0 | 3.358 | 5.185 | 0.747 | 2.724 | 0 | 0.812 |
| OCBBF2000904 | 0 | 0 | 14.319 | 4.124 | 0 | 0 | 0 |
| OCBBF2000982 | 0.872 | 0 | 3.089 | 1.779 | 0 | 0 | 1.934 |
| OCBBF2000986 | 1.927 | 2.211 | 0 | 0 | 0 | 0 | 0 |
| OCBBF2001140 | 16.961 | 0 | 0 | 23.073 | 0 | 0 | 12.540 |
| OCBBF2001166 | 16.442 | 0 | 0 | 5.592 | 0 | 0 | 0 |
| OCBBF2001210 | 7.252 | 0 | 0 | 22.195 | 0 | 0 | 0 |
| OCBBF2001307 | 11.643 | 0 | 11.784 | 27.151 | 0 | 0 | 25.823 |
| OCBBF2001408 | 0 | 0 | 0 | 39.401 | 0 | 0 | 0 |
| OCBBF2001416 | 20.868 | 0 | 0 | 0 | 0 | 0 | 0 |
| OCBBF2001492 | 4.117 | 1.574 | 2.431 | 0 | 0 | 2.474 | 6.087 |
| OCBBF2001586 | 3.586 | 0 | 0 | 3.658 | 0 | 0 | 3.976 |
| OCBBF2001639 | 0 | 10.788 | 0 | 4.798 | 0 | 16.956 | 5.215 |
| OCBBF2001687 | 3.847 | 0 | 6.814 | 1.962 | 0 | 0 | 0 |
| OCBBF2001794 | 4.728 | 0 | 0 | 0 | 0 | 0 | 0 |
| OCBBF2001931 | 0 | 0 | 0 | 0 | 62.325 | 0 | 0 |
| OCBBF2002015 | 0 | 0 | 0 | 0 | 0 | 0 | 15.338 |
| OCBBF2002083 | 0 | 25.211 | 0 | 0 | 0 | 0 | 0 |
| OCBBF2002086 | 3.222 | 0 | 0 | 0 | 0 | 0 | 0 |
| OCBBF2002290 | 0 | 0 | 0 | 0 | 0 | 0 | 11.184 |
| OCBBF2002626 | 1.689 | 0 | 0 | 2.585 | 0 | 0 | 0 |
| OCBBF2002656 | 1.654 | 0 | 0 | 1.688 | 6.157 | 0 | 0 |
| OCBBF2002980 | 0 | 0 | 0 | 45.093 | 0 | 0 | 0 |
| OCBBF2003052 | 11.395 | 0 | 0 | 7.750 | 0 | 0 | 0 |
| OCBBF2003246 | 9.786 | 0 | 0 | 19.969 | 0 | 0 | 0 |
| OCBBF2003518 | 2.062 | 0 | 0 | 0 | 0 | 0 | 0 |
| OCBBF2003593 | 44.597 | 0 | 0 | 0 | 0 | 0 | 0 |
| OCBBF2003819 | 0 | 0 | 0 | 21.496 | 0 | 0 | 0 |
| OCBBF2003925 | 0 | 0 | 0 | 0 | 0 | 3.004 | 0 |
| OCBBF2004104 | 5.478 | 3.142 | 0 | 0 | 5.098 | 0 | 3.038 |
| OCBBF2004259 | 0 | 0 | 0 | 0 | 0 | 0 | 47.165 |
| OCBBF2004273 | 0 | 0 | 0 | 3.658 | 0 | 0 | 0.994 |
| OCBBF2004533 | 0 | 0 | 0 | 0 | 74.975 | 0 | 0 |
| OCBBF2004669 | 0 | 0 | 0 | 45.093 | 0 | 0 | 0 |
| OCBBF2004757 | 0 | 5.686 | 0 | 0 | 0 | 0 | 0 |
| OCBBF2004889 | 44.597 | 0 | 0 | 0 | 0 | 0 | 0 |
| OCBBF2004930 | 0 | 23.811 | 0 | 0 | 0 | 37.423 | 0 |
| OCBBF2004984 | 0 | 0 | 0 | 0 | 0 | 0 | 26.412 |
| OCBBF2005161 | 0 | 0 | 0 | 45.093 | 0 | 0 | 0 |
| OCBBF2005420 | 21.156 | 0 | 0 | 0 | 0 | 0 | 0 |
| OCBBF2005428 | 0 | 7.563 | 0 | 0 | 0 | 0 | 0 |
| OCBBF2005546 | 0 | 0 | 0 | 45.093 | 0 | 0 | 0 |
| OCBBF2005956 | 0 | 6.513 | 0 | 0 | 10.566 | 0 | 3.148 |
| OCBBF2006005 | 9.535 | 0 | 0 | 9.728 | 0 | 0 | 21.147 |
| OCBBF2006148 | 0 | 0 | 0 | 0 | 0 | 0 | 7.510 |
| OCBBF2006151 | 0 | 0 | 20.423 | 11.764 | 0 | 0 | 0 |
| OCBBF2006172 | 0 | 12.029 | 18.574 | 0 | 19.515 | 0 | 0 |
| OCBBF2006313 | 0 | 13.100 | 0 | 0 | 0 | 0 | 0 |
| OCBBF2006660 | 0 | 0 | 0 | 15.400 | 0 | 0 | 0 |
| OCBBF2006764 | 15.345 | 0 | 0 | 0 | 0 | 0 | 0 |
| OCBBF2006972 | 44.597 | 0 | 0 | 0 | 0 | 0 | 0 |
| OCBBF2006987 | 0 | 0 | 0 | 13.187 | 0 | 0 | 0 |
| OCBBF2007028 | 9.665 | 0 | 0 | 11.269 | 0 | 0 | 18.374 |
| OCBBF2007051 | 0 | 0 | 0 | 45.093 | 0 | 0 | 0 |
| OCBBF2007068 | 0 | 0 | 0 | 45.093 | 0 | 0 | 0 |
| OCBBF2007224 | 9.442 | 0 | 0 | 0 | 0 | 0 | 0 |
| OCBBF2007354 | 3.014 | 2.305 | 0 | 0 | 0 | 0 | 1.114 |
| OCBBF2007415 | 4.004 | 9.185 | 0 | 7.353 | 2.980 | 0 | 10.656 |
| OCBBF2007762 | 0 | 0 | 0 | 16.042 | 0 | 0 | 0 |
| OCBBF2007829 | 10.981 | 0 | 1.945 | 8.963 | 2.044 | 0 | 3.653 |
| OCBBF2007892 | 0 | 0 | 0 | 0 | 0 | 15.360 | 0 |
| OCBBF2008005 | 0 | 0 | 0 | 1.620 | 0 | 0 | 0 |
| OCBBF2008041 | 4.180 | 0 | 0 | 0 | 0 | 0 | 0 |
| OCBBF2008144 | 5.768 | 0 | 0 | 2.942 | 0 | 0 | 3.198 |
| OCBBF2008340 | 0 | 0 | 0 | 15.751 | 0 | 0 | 0 |
| OCBBF2008466 | 0 | 0 | 0 | 0 | 31.840 | 0 | 0 |
| OCBBF2008483 | 0 | 0 | 0 | 0 | 0 | 31.388 | 0 |
| OCBBF2008701 | 21.675 | 0 | 0 | 0 | 0 | 0 | 0 |
| OCBBF2009095 | 19.717 | 0 | 0 | 0 | 0 | 0 | 0 |
| OCBBF2009242 | 2.831 | 0 | 0 | 8.666 | 10.539 | 0 | 12.560 |
| OCBBF2009301 | 0 | 0 | 0 | 2.115 | 0 | 0 | 0 |
| OCBBF2009352 | 0 | 0 | 0 | 11.486 | 0 | 0 | 12.484 |
| OCBBF2009536 | 0 | 0 | 0 | 0 | 0 | 0 | 47.165 |
| OCBBF2009772 | 9.420 | 2.702 | 4.172 | 12.014 | 0 | 4.246 | 1.306 |
| OCBBF2009836 | 4.098 | 0 | 0 | 4.180 | 0 | 0 | 4.544 |
| OCBBF2009920 | 0 | 0 | 0 | 45.093 | 0 | 0 | 0 |
| OCBBF2010140 | 32.508 | 0 | 0 | 0 | 0 | 0 | 0 |
| OCBBF2010819 | 0 | 0 | 0 | 0 | 0 | 0 | 13.910 |
| OCBBF2010830 | 0 | 0 | 0 | 0.898 | 0 | 0 | 0.976 |
| OCBBF2010841 | 6.216 | 0 | 0 | 6.342 | 0 | 0 | 6.893 |
| OCBBF2010858 | 13.322 | 0 | 0 | 20.388 | 0 | 0 | 0 |
| OCBBF2010871 | 9.142 | 0 | 0 | 9.327 | 0 | 0 | 5.069 |
| OCBBF2011021 | 27.462 | 0 | 0 | 19.170 | 0 | 0 | 1.603 |
| OCBBF2011073 | 6.824 | 0 | 0 | 3.481 | 0 | 0 | 3.784 |
| OCBBF2011160 | 17.456 | 0 | 0 | 0 | 0 | 0 | 0 |
| OCBBF2011228 | 21.380 | 0 | 0 | 0 | 0 | 0 | 0 |
| OCBBF2011232 | 8.012 | 0 | 0 | 0 | 0 | 0 | 8.885 |
| OCBBF2011283 | 5.923 | 0 | 0 | 0 | 0 | 0 | 13.137 |
| OCBBF2011536 | 1.995 | 4.577 | 0 | 0 | 0 | 0 | 0 |
| OCBBF2011759 | 11.214 | 0 | 0 | 24.789 | 0 | 0 | 19.690 |
| OCBBF2011767 | 29.740 | 0 | 0 | 0 | 0 | 0 | 0 |
| OCBBF2011887 | 0 | 0 | 0 | 4.798 | 17.504 | 0 | 5.215 |
| OCBBF2011897 | 0 | 0 | 61.158 | 0 | 0 | 0 | 0 |
| OCBBF2011914 | 3.646 | 0 | 0 | 0 | 0 | 0 | 0 |
| OCBBF2012001 | 24.900 | 0 | 0 | 0 | 0 | 0 | 0 |
| OCBBF2012039 | 26.074 | 0 | 0 | 31.296 | 0 | 0 | 22.111 |
| OCBBF2012095 | 0 | 0 | 0 | 15.612 | 0 | 0 | 0 |
| OCBBF2012262 | 29.008 | 0 | 0 | 0 | 0 | 0 | 0 |
| OCBBF2012553 | 0 | 2.691 | 0 | 2.393 | 4.365 | 4.229 | 0 |
| OCBBF2012678 | 6.898 | 0 | 0 | 0 | 0 | 0 | 0 |
| OCBBF2012704 | 0 | 0 | 0 | 21.301 | 0 | 0 | 0 |
| OCBBF2012908 | 3.277 | 0 | 0 | 6.686 | 12.196 | 11.816 | 0 |
| OCBBF2012936 | 44.597 | 0 | 0 | 0 | 0 | 0 | 0 |
| OCBBF2013011 | 0 | 0 | 0 | 7.703 | 0 | 0 | 0 |
| OCBBF2013123 | 22.817 | 0 | 0 | 23.279 | 0 | 0 | 0 |
| OCBBF2013149 | 3.102 | 0 | 0 | 6.330 | 0 | 5.593 | 5.160 |
| OCBBF2013366 | 0 | 0 | 0 | 2.754 | 0 | 0 | 2.993 |
| OCBBF2013481 | 21.868 | 0 | 0 | 11.155 | 40.696 | 0 | 0 |
| OCBBF2013789 | 0 | 34.623 | 0 | 0 | 0 | 0 | 0 |
| OCBBF2013843 | 5.343 | 1.226 | 0 | 5.451 | 1.989 | 0 | 6.518 |
| OCBBF2014064 | 0 | 0 | 0 | 4.180 | 10.167 | 4.925 | 0 |
| OCBBF2014089 | 19.735 | 0 | 0 | 0 | 0 | 0 | 0 |
| OCBBF2014292 | 0 | 0 | 0 | 0 | 0 | 0 | 2.297 |
| OCBBF2014322 | 0 | 0 | 0 | 0 | 0 | 0 | 22.893 |
| OCBBF2014541 | 3.921 | 0 | 0 | 0 | 0 | 0 | 0 |
| OCBBF2014576 | 0 | 0 | 0 | 3.728 | 0 | 0 | 4.052 |
| OCBBF2014873 | 28.366 | 0 | 0 | 0 | 0 | 0 | 0 |
| OCBBF2015503 | 0 | 0 | 0 | 0 | 0 | 0 | 8.322 |
| OCBBF2015931 | 0 | 0 | 0 | 0 | 74.975 | 0 | 0 |
| OCBBF2016729 | 9.116 | 0 | 0 | 0 | 0 | 0 | 0 |
| OCBBF2017035 | 0 | 2.781 | 0 | 3.711 | 4.512 | 4.371 | 5.378 |
| OCBBF2017306 | 44.597 | 0 | 0 | 0 | 0 | 0 | 0 |
| OCBBF2017398 | 9.005 | 0 | 4.557 | 6.562 | 4.788 | 0 | 4.280 |
| OCBBF2017754 | 0 | 0 | 0 | 7.661 | 0 | 0 | 0 |
| OCBBF2017791 | 1.281 | 2.939 | 0 | 5.227 | 0 | 0 | 5.682 |
| OCBBF2017882 | 7.135 | 0 | 0 | 7.279 | 53.111 | 0 | 7.912 |
| OCBBF2017888 | 17.037 | 0 | 0 | 0 | 0 | 0 | 0 |
| OCBBF2017893 | 0 | 0 | 0 | 0 | 12.912 | 0 | 0 |
| OCBBF2017920 | 0 | 0 | 0 | 0 | 0 | 0 | 28.558 |
| OCBBF2018084 | 4.410 | 0 | 0 | 0 | 0 | 0 | 0 |
| OCBBF2018206 | 0 | 0 | 0 | 45.093 | 0 | 0 | 0 |
| OCBBF2018234 | 19.431 | 0 | 0 | 0 | 0 | 0 | 0 |
| OCBBF2018581 | 0 | 0 | 0 | 0 | 16.013 | 0 | 0 |
| OCBBF2018618 | 16.218 | 0 | 0 | 16.546 | 0 | 0 | 0 |
| OCBBF2018707 | 0 | 0 | 0 | 0 | 0 | 16.218 | 0 |
| OCBBF2018827 | 9.813 | 0 | 0 | 15.018 | 0 | 0 | 5.441 |
| OCBBF2018873 | 1.386 | 0 | 0 | 2.828 | 0 | 0 | 4.610 |
| OCBBF2018956 | 0 | 0 | 0 | 0 | 0 | 0 | 1.877 |
| OCBBF2019950 | 0 | 0 | 0 | 45.093 | 0 | 0 | 0 |
| OCBBF2020741 | 0 | 0 | 0 | 28.789 | 0 | 0 | 0 |
| OCBBF2021286 | 18.456 | 0 | 0 | 18.829 | 0 | 0 | 10.233 |
| OCBBF2021833 | 14.503 | 0 | 0 | 0 | 0 | 0 | 32.166 |
| OCBBF2024719 | 0 | 0 | 0 | 30.261 | 0 | 0 | 32.892 |
| OCBBF2024850 | 4.445 | 0 | 0 | 4.535 | 0 | 8.013 | 7.393 |
| OCBBF2028055 | 44.597 | 0 | 0 | 0 | 0 | 0 | 0 |
| OCBBF2030116 | 0 | 0 | 0 | 45.093 | 0 | 0 | 0 |
| OCBBF2031561 | 0 | 0 | 0 | 0 | 0 | 0 | 34.817 |
| OCBBF2033295 | 10.516 | 0 | 0 | 10.729 | 0 | 0 | 0 |
| OCBBF2035226 | 0 | 0 | 0 | 0.599 | 0 | 4.236 | 0 |
| OCBBF2036743 | 11.053 | 0 | 0 | 0 | 0 | 0 | 12.257 |
| OCBBF2038317 | 19.713 | 0 | 0 | 0 | 0 | 0 | 10.930 |
| OCBBF3000296 | 0 | 0 | 0 | 19.328 | 0 | 0 | 0 |
| OCBBF3000323 | 0 | 0 | 0 | 3.393 | 0 | 0 | 0 |
| OCBBF3001333 | 9.639 | 0 | 0 | 0 | 0 | 0 | 0 |
| OCBBF3003103 | 44.597 | 0 | 0 | 0 | 0 | 0 | 0 |
| OCBBF3004487 | 4.531 | 0 | 0 | 4.622 | 0 | 0 | 0 |
| OCBBF3008230 | 29.840 | 0 | 0 | 0 | 0 | 0 | 33.091 |
| OCBBF3008835 | 0 | 0 | 0 | 0 | 0 | 0 | 47.165 |
| PEBLM1000144 | 5.997 | 0 | 0 | 0 | 0 | 0 | 0 |
| PEBLM2000030 | 0 | 0 | 0 | 0 | 0 | 0 | 4.081 |
| PEBLM2000147 | 0 | 2.927 | 0 | 1.302 | 0 | 0 | 1.415 |
| PEBLM2000222 | 0 | 1.333 | 2.058 | 0 | 0 | 0 | 0.644 |
| PEBLM2000267 | 2.812 | 0 | 0 | 1.435 | 0 | 20.280 | 1.559 |
| PEBLM2000308 | 1.982 | 9.096 | 0 | 0 | 0 | 0 | 0 |
| PEBLM2000326 | 10.856 | 0 | 0 | 0 | 0 | 0 | 0 |
| PEBLM2000395 | 0.910 | 0 | 0 | 0 | 0 | 0 | 1.009 |
| PEBLM2001072 | 10.881 | 4.160 | 0 | 1.850 | 0 | 0 | 2.011 |
| PEBLM2002455 | 0 | 0 | 0 | 0 | 33.118 | 0 | 0 |
| PEBLM2003914 | 2.125 | 0 | 7.527 | 2.168 | 0 | 7.661 | 2.356 |
| PEBLM2004452 | 10.797 | 0 | 0 | 12.393 | 0 | 0 | 5.987 |
| PEBLM2004666 | 3.715 | 0 | 0 | 0 | 0 | 0 | 0 |
| PEBLM2004733 | 0 | 21.924 | 0 | 0 | 0 | 0 | 0 |
| PEBLM2006283 | 0 | 0 | 0 | 11.951 | 0 | 0 | 0 |
| PEBLM2006366 | 0 | 6.350 | 0 | 0 | 10.301 | 0 | 0 |
| PEBLM2007774 | 0.301 | 0.691 | 0 | 0.307 | 1.121 | 0 | 0 |
| PEBLM2008826 | 0 | 0 | 0 | 0 | 0 | 0 | 10.285 |
| PERIC2000473 | 6.277 | 0 | 44.476 | 0 | 0 | 0 | 6.961 |
| PERIC2007068 | 3.086 | 7.080 | 0 | 4.723 | 0 | 0 | 1.711 |
| PLACE5000013 | 0 | 0 | 0 | 0 | 0 | 0 | 1.760 |
| PLACE5000058 | 0 | 0 | 0 | 0 | 0 | 0 | 38.390 |
| PLACE5000066 | 0 | 0 | 0 | 0 | 0 | 32.991 | 0 |
| PLACE5000113 | 6.882 | 0 | 0 | 3.511 | 0 | 0 | 1.908 |
| PLACE5000159 | 6.818 | 0 | 0 | 13.912 | 0 | 0 | 2.520 |
| PLACE5000170 | 0 | 0 | 0 | 3.547 | 0 | 0 | 7.711 |
| PLACE5000172 | 6.499 | 0 | 0 | 11.935 | 0 | 0 | 7.207 |
| PLACE5000178 | 35.975 | 0 | 0 | 0 | 0 | 0 | 0 |
| PLACE5000297 | 0 | 0 | 0 | 36.438 | 0 | 0 | 0 |
| PLACE5000492 | 0 | 56.315 | 0 | 0 | 0 | 0 | 0 |
| PLACE6000263 | 1.520 | 0 | 5.386 | 9.307 | 0 | 0 | 3.372 |
| PLACE6000314 | 0 | 0 | 0 | 0 | 0 | 0 | 16.914 |
| PLACE6000325 | 0 | 18.585 | 0 | 0 | 0 | 0 | 8.984 |
| PLACE6000379 | 0.177 | 0.405 | 1.251 | 0.901 | 0 | 1.274 | 0.783 |
| PLACE6000414 | 0 | 0 | 0 | 0 | 0 | 0 | 1.421 |
| PLACE6000463 | 0 | 17.901 | 0 | 0 | 0 | 0 | 0 |
| PLACE6001064 | 4.590 | 0 | 0 | 7.609 | 0 | 2.069 | 3.181 |
| PLACE6001185 | 21.358 | 0 | 0 | 0 | 0 | 0 | 0 |
| PLACE6001281 | 3.443 | 0 | 0 | 7.024 | 0 | 0 | 3.818 |
| PLACE6001443 | 0 | 0 | 0 | 2.004 | 0 | 0 | 2.178 |
| PLACE6001823 | 0 | 0 | 0 | 29.069 | 0 | 0 | 0 |
| PLACE6002084 | 0 | 0.847 | 0 | 1.130 | 0 | 0 | 0 |
| PLACE6002102 | 4.237 | 3.240 | 0 | 12.968 | 0 | 0 | 7.831 |
| PLACE6002668 | 0 | 0 | 0 | 0 | 0 | 0 | 5.985 |
| PLACE6003004 | 0 | 0 | 0 | 0 | 0 | 0 | 38.390 |
| PLACE6003204 | 21.933 | 0 | 0 | 0 | 0 | 0 | 0 |
| PLACE6003218 | 14.551 | 0 | 0 | 0 | 0 | 0 | 0 |
| PLACE6003383 | 1.194 | 0 | 0 | 0 | 0 | 0 | 0 |
| PLACE6003745 | 0 | 0 | 0 | 5.361 | 0 | 0 | 5.827 |
| PLACE6003850 | 0 | 0 | 0 | 0 | 0 | 40.449 | 0 |
| PLACE6004005 | 2.962 | 0 | 0 | 2.015 | 0 | 0 | 1.095 |
| PLACE6004312 | 1.206 | 0 | 0 | 1.231 | 0 | 2.175 | 0 |
| PLACE6004397 | 1.838 | 8.434 | 13.023 | 0 | 6.841 | 0 | 2.038 |
| PLACE6004578 | 0 | 0 | 0 | 0 | 4.614 | 0 | 0 |
| PLACE6005231 | 0 | 25.339 | 0 | 0 | 0 | 0 | 0 |
| PLACE6005294 | 0 | 0 | 42.656 | 0 | 0 | 0 | 0 |
| PLACE6005482 | 2.644 | 6.066 | 0 | 5.395 | 9.842 | 0 | 2.932 |
| PLACE6006137 | 10.247 | 7.836 | 0 | 17.423 | 0 | 0 | 7.575 |
| PLACE6006186 | 0 | 0 | 0 | 7.379 | 0 | 0 | 0 |
| PLACE6006222 | 8.355 | 0 | 29.599 | 17.049 | 0 | 30.127 | 0 |
| PLACE6006418 | 0 | 0 | 0 | 0 | 0 | 0 | 6.149 |
| PLACE6007180 | 21.434 | 0 | 0 | 0 | 0 | 0 | 11.885 |
| PLACE6007309 | 0 | 0 | 0 | 0 | 67.652 | 0 | 0 |
| PLACE6007373 | 0 | 0 | 0 | 0 | 0 | 0 | 3.174 |
| PLACE6007436 | 0 | 0 | 12.727 | 3.665 | 0 | 0 | 0 |
| PLACE6007925 | 0 | 23.248 | 0 | 0 | 0 | 0 | 0 |
| PLACE6008775 | 3.686 | 0 | 6.529 | 16.924 | 0 | 0 | 8.176 |
| PLACE6009821 | 0 | 0 | 0 | 0 | 0 | 55.521 | 17.076 |
| PLACE6010077 | 0 | 0 | 0 | 0 | 0 | 0 | 0.966 |
| PLACE6010484 | 0 | 0 | 0 | 0 | 0 | 5.573 | 0 |
| PLACE6010704 | 35.975 | 0 | 0 | 0 | 0 | 0 | 0 |
| PLACE6010732 | 0 | 56.315 | 0 | 0 | 0 | 0 | 0 |
| PLACE6011057 | 4.508 | 0 | 0 | 2.299 | 8.389 | 0 | 0 |
| PLACE6011156 | 0 | 29.874 | 0 | 0 | 0 | 46.952 | 0 |
| PLACE6012028 | 0 | 0 | 0 | 0 | 0 | 0 | 6.961 |
| PLACE6013883 | 0 | 0 | 0 | 0 | 0 | 0 | 38.390 |
| PLACE6014064 | 0 | 0 | 0 | 0 | 0 | 0 | 3.394 |
| PLACE6015051 | 0 | 0 | 0 | 0 | 17.157 | 0 | 0 |
| PLACE6015445 | 0 | 0 | 0 | 0 | 0 | 40.069 | 0 |
| PLACE6015731 | 0 | 0 | 54.376 | 0 | 0 | 0 | 0 |
| PLACE6016942 | 3.912 | 8.975 | 13.858 | 0 | 14.560 | 0 | 4.338 |
| PLACE6016984 | 0 | 0 | 0 | 18.846 | 0 | 0 | 0 |
| PLACE6017701 | 0 | 56.315 | 0 | 0 | 0 | 0 | 0 |
| PLACE6018031 | 0 | 0 | 0 | 0 | 0 | 0 | 7.456 |
| PLACE6019195 | 8.485 | 0 | 0 | 8.657 | 31.582 | 0 | 0 |
| PLACE7000410 | 0 | 0 | 0 | 0 | 0 | 0 | 38.390 |
| PLACE7004103 | 3.880 | 0 | 0 | 5.278 | 0 | 0 | 4.302 |
| PLACE7006240 | 25.174 | 0 | 0 | 0 | 0 | 0 | 0 |
| PLACE7008261 | 0 | 0 | 0 | 29.069 | 0 | 0 | 0 |
| PROST1000085 | 0 | 0 | 0 | 0 | 51.498 | 0 | 0 |
| PROST1000097 | 5.292 | 0 | 0 | 6.599 | 4.377 | 0 | 1.956 |
| PROST1000144 | 1.568 | 0 | 0 | 0 | 0 | 0 | 0 |
| PROST1000199 | 0 | 0 | 0 | 0.745 | 0 | 0 | 1.621 |
| PROST1000383 | 0 | 0 | 0 | 0 | 0 | 0 | 23.821 |
| PROST1000526 | 5.053 | 5.796 | 0 | 0 | 0 | 0 | 2.802 |
| PROST1000564 | 2.688 | 0 | 0 | 0 | 0 | 0 | 0 |
| PROST2000138 | 0.087 | 0 | 0.614 | 0.177 | 0.323 | 0 | 0.096 |
| PROST2000206 | 0 | 0 | 0 | 22.341 | 0 | 0 | 0 |
| PROST2000463 | 0 | 39.281 | 0 | 0 | 0 | 0 | 0 |
| PROST2000760 | 0 | 0 | 0 | 0 | 0 | 0 | 6.222 |
| PROST2001180 | 9.932 | 5.696 | 0 | 2.533 | 9.241 | 0 | 2.753 |
| PROST2001283 | 0 | 0 | 0 | 0 | 0 | 7.672 | 0 |
| PROST2001997 | 5.830 | 13.376 | 0 | 0 | 0 | 0 | 0 |
| PROST2002651 | 0 | 0 | 0 | 0 | 11.130 | 0 | 0 |
| PROST2003232 | 2.047 | 0 | 2.417 | 2.088 | 0 | 0 | 1.513 |
| PROST2003324 | 17.682 | 0 | 0 | 0 | 0 | 0 | 19.609 |
| PROST2003338 | 2.239 | 7.704 | 0 | 1.142 | 0 | 0 | 2.483 |
| PROST2003396 | 5.974 | 0 | 0 | 0 | 0 | 0 | 0 |
| PROST2003517 | 2.633 | 0 | 0 | 0 | 0 | 0 | 0 |
| PROST2004095 | 9.361 | 2.148 | 0 | 5.253 | 1.742 | 1.688 | 3.114 |
| PROST2004258 | 0 | 0 | 13.516 | 0 | 0 | 0 | 0 |
| PROST2004270 | 0.588 | 2.698 | 0 | 1.200 | 0 | 0 | 1.304 |
| PROST2004416 | 0 | 0 | 0 | 0 | 0 | 0 | 2.262 |
| PROST2004739 | 0 | 0 | 0 | 7.587 | 0 | 0 | 0 |
| PROST2005067 | 0 | 0 | 0 | 0 | 0 | 0 | 17.889 |
| PROST2005076 | 0 | 0 | 0 | 0 | 0 | 25.962 | 0 |
| PROST2005272 | 8.212 | 0 | 0 | 0 | 0 | 0 | 0 |
| PROST2005604 | 6.221 | 0 | 0 | 3.173 | 0 | 0 | 0 |
| PROST2005793 | 0 | 0 | 0 | 0 | 0 | 0 | 4.349 |
| PROST2006030 | 15.135 | 0 | 0 | 0 | 0 | 0 | 4.196 |
| PROST2006260 | 4.072 | 0 | 0 | 0 | 0 | 0 | 4.516 |
| PROST2006988 | 0 | 33.501 | 0 | 0 | 0 | 0 | 0 |
| PROST2007248 | 0 | 0 | 0 | 0 | 0 | 0 | 20.670 |
| PROST2007612 | 3.193 | 0 | 0 | 0 | 0 | 0 | 3.541 |
| PROST2008271 | 0 | 0 | 0 | 7.997 | 0 | 0 | 0 |
| PROST2008360 | 1.800 | 0 | 0 | 1.836 | 0 | 0 | 3.992 |
| PROST2008489 | 0 | 0 | 2.294 | 0 | 0 | 0 | 0.718 |
| PROST2008770 | 4.680 | 0 | 0 | 6.367 | 2.903 | 2.813 | 3.460 |
| PROST2009347 | 0 | 0 | 0 | 19.336 | 0 | 0 | 0 |
| PROST2009731 | 0 | 2.488 | 0 | 0 | 0 | 0 | 0 |
| PROST2010782 | 0.243 | 0 | 0 | 0 | 0 | 0 | 0 |
| PROST2011038 | 0 | 27.530 | 0 | 0 | 0 | 0 | 0 |
| PROST2011577 | 4.116 | 2.361 | 0 | 1.050 | 3.830 | 0 | 0 |
| PROST2012007 | 1.135 | 5.208 | 0 | 0 | 0 | 0 | 0 |
| PROST2012353 | 0 | 0 | 0 | 0 | 0 | 33.893 | 0 |
| PROST2012448 | 16.318 | 0 | 0 | 0 | 0 | 0 | 0 |
| PROST2012745 | 0 | 0 | 13.700 | 0 | 0 | 0 | 0 |
| PROST2015251 | 0 | 0 | 0 | 0 | 0 | 0 | 23.821 |
| PROST2015332 | 10.310 | 0 | 18.262 | 5.259 | 0 | 0 | 0 |
| PROST2015464 | 0 | 0 | 0 | 0 | 0 | 0 | 5.706 |
| PROST2015710 | 0 | 0 | 0 | 0 | 0 | 0 | 13.657 |
| PROST2016195 | 0 | 0 | 0 | 0 | 0 | 21.086 | 0 |
| PROST2016918 | 6.355 | 0 | 0 | 6.484 | 0 | 0 | 14.095 |
| PROST2017529 | 0 | 0 | 0 | 0 | 0 | 0 | 2.815 |
| PROST2017692 | 0 | 0 | 0 | 0.752 | 0 | 2.657 | 0 |
| PROST2017972 | 12.094 | 0 | 0 | 0 | 45.015 | 0 | 0 |
| PROST2018583 | 0 | 0 | 0 | 0 | 0 | 33.325 | 0 |
| PROST2018588 | 0 | 30.107 | 0 | 0 | 0 | 0 | 0 |
| PROST2018977 | 0 | 0 | 0 | 0 | 0 | 0 | 14.411 |
| PROST2019164 | 3.687 | 0 | 0 | 0 | 0 | 0 | 0 |
| PROST2019253 | 0 | 0 | 0 | 0 | 0 | 0 | 1.863 |
| PUAEN1000057 | 2.053 | 4.710 | 0 | 0 | 0 | 7.403 | 11.384 |
| PUAEN1000065 | 0.670 | 6.146 | 2.373 | 0 | 2.493 | 0 | 0 |
| PUAEN1000147 | 9.325 | 0 | 0 | 0 | 0 | 0 | 0 |
| PUAEN1000161 | 5.768 | 0 | 0 | 0 | 0 | 0 | 0 |
| PUAEN1000164 | 0.398 | 0 | 0 | 0 | 0 | 0 | 1.324 |
| PUAEN1000239 | 0 | 0 | 0 | 3.163 | 0 | 0 | 0 |
| PUAEN1000322 | 0 | 0 | 4.529 | 0 | 0 | 0 | 0 |
| PUAEN2000152 | 0 | 0 | 0 | 6.055 | 0 | 0 | 6.582 |
| PUAEN2000312 | 0 | 0 | 0 | 0 | 0 | 0 | 9.818 |
| PUAEN2000374 | 2.240 | 0 | 0 | 0.653 | 0 | 0 | 1.775 |
| PUAEN2000535 | 0.804 | 0 | 0 | 0 | 0 | 0 | 0 |
| PUAEN2001345 | 0 | 0 | 0 | 0 | 0 | 0 | 3.247 |
| PUAEN2001586 | 0 | 0 | 0 | 2.913 | 0 | 0 | 0 |
| PUAEN2002489 | 0 | 0 | 0 | 0 | 0 | 0 | 21.707 |
| PUAEN2003018 | 0 | 0 | 0 | 0 | 0 | 12.086 | 3.717 |
| PUAEN2003408 | 0 | 6.909 | 0 | 0 | 0 | 0 | 0 |
| PUAEN2005930 | 18.362 | 0 | 0 | 9.367 | 0 | 0 | 10.181 |
| PUAEN2006335 | 12.682 | 0 | 0 | 0 | 0 | 0 | 0 |
| PUAEN2007044 | 8.600 | 0 | 0 | 0 | 0 | 0 | 1.590 |
| PUAEN2008515 | 0 | 0 | 0 | 9.905 | 0 | 0 | 21.533 |
| PUAEN2008985 | 4.437 | 0 | 0 | 4.527 | 0 | 0 | 0 |
| PUAEN2009655 | 18.275 | 0 | 0 | 7.458 | 0 | 0 | 0 |
| PUAEN2010346 | 0 | 0 | 35.358 | 0 | 0 | 0 | 0 |
| PUAEN2010824 | 5.239 | 0 | 0 | 16.034 | 0 | 0 | 11.619 |
| RECTM2001347 | 0 | 0 | 0 | 0 | 0 | 0 | 4.600 |
| SALGL1000005 | 0 | 0 | 0 | 0 | 0 | 1.093 | 0 |
| SALGL1000047 | 0.296 | 0 | 0 | 0.302 | 0 | 0 | 0 |
| SALGL1000157 | 0 | 0 | 0.461 | 0.265 | 0.968 | 0 | 0.577 |
| SKMUS1000014 | 7.356 | 0 | 0 | 0 | 0 | 0 | 0 |
| SKMUS1000022 | 0.832 | 1.910 | 2.949 | 0 | 0 | 0 | 0 |
| SKMUS1000084 | 1.540 | 3.533 | 0 | 3.142 | 5.732 | 0 | 0 |
| SKMUS1000102 | 9.671 | 22.187 | 0 | 0 | 0 | 0 | 0 |
| SKMUS1000110 | 0 | 0 | 0 | 0 | 0 | 0 | 8.170 |
| SKMUS1000176 | 0 | 0 | 0 | 0 | 3.211 | 0 | 0 |
| SKMUS1000177 | 2.361 | 0 | 0 | 2.409 | 0 | 0 | 0 |
| SKMUS1000186 | 0.803 | 1.842 | 1.422 | 0.819 | 1.494 | 1.447 | 0 |
| SKMUS2000020 | 0 | 0 | 0 | 0 | 0 | 13.280 | 0 |
| SKMUS2000117 | 0 | 0 | 0 | 0 | 0 | 0 | 9.423 |
| SKMUS2000271 | 0 | 0 | 0 | 0 | 0 | 45.999 | 0 |
| SKMUS2000361 | 0 | 2.453 | 0 | 0 | 0 | 0 | 0 |
| SKMUS2000380 | 0 | 4.975 | 0 | 0 | 0 | 0 | 0 |
| SKMUS2000484 | 0 | 0 | 0 | 1.792 | 0 | 0 | 1.947 |
| SKMUS2000757 | 0 | 0 | 0 | 0 | 0 | 0 | 5.480 |
| SKMUS2000873 | 1.100 | 0 | 0 | 0.561 | 0 | 0 | 0 |
| SKMUS2000931 | 1.632 | 1.248 | 0 | 0.555 | 0 | 0 | 4.827 |
| SKMUS2000954 | 0 | 3.899 | 0 | 0 | 0 | 0 | 0 |
| SKMUS2000961 | 0 | 0 | 0 | 0 | 0 | 0 | 7.301 |
| SKMUS2001147 | 2.772 | 0 | 0 | 2.828 | 0 | 0 | 3.074 |
| SKMUS2001454 | 2.025 | 0.774 | 0 | 0.689 | 2.512 | 1.217 | 2.620 |
| SKMUS2001528 | 0 | 0 | 0 | 0 | 9.344 | 0 | 0 |
| SKMUS2001634 | 1.309 | 0 | 0 | 0 | 0 | 0 | 1.452 |
| SKMUS2004908 | 6.569 | 0 | 0 | 3.351 | 0 | 0 | 7.285 |
| SKNMC1000110 | 0 | 0 | 0 | 0 | 0 | 0 | 4.015 |
| SKNMC1000159 | 0 | 0 | 0 | 10.774 | 0 | 0 | 0 |
| SKNMC1000168 | 1.675 | 0 | 0 | 0 | 0 | 0 | 0 |
| SKNMC2000065 | 3.239 | 0 | 0 | 1.101 | 4.018 | 0 | 10.774 |
| SKNMC2000097 | 0 | 0 | 0 | 0.455 | 0 | 0 | 0.494 |
| SKNMC2000305 | 0 | 0 | 0 | 14.374 | 0 | 0 | 7.812 |
| SKNMC2000322 | 1.412 | 0 | 0 | 0 | 0 | 0 | 0 |
| SKNMC2000635 | 0 | 0.412 | 0 | 0 | 0 | 0 | 0.796 |
| SKNMC2000649 | 0 | 0 | 0 | 0.931 | 0 | 0 | 0 |
| SKNMC2000877 | 1.185 | 0 | 0 | 2.418 | 0 | 8.545 | 0 |
| SKNMC2001503 | 0 | 0 | 0 | 0 | 11.542 | 11.182 | 3.439 |
| SKNMC2006173 | 0 | 0 | 0 | 7.851 | 0 | 0 | 0 |
| SKNSH1000002 | 1.437 | 0 | 0 | 0 | 0 | 0 | 0 |
| SKNSH1000101 | 1.285 | 1.474 | 0 | 1.966 | 2.391 | 0 | 0 |
| SKNSH2000101 | 13.179 | 0 | 0 | 0 | 0 | 0 | 7.308 |
| SKNSH2000250 | 14.088 | 9.697 | 0 | 15.811 | 15.731 | 5.080 | 4.687 |
| SKNSH2000347 | 4.487 | 0 | 0 | 4.578 | 0 | 0 | 4.976 |
| SKNSH2000716 | 0 | 3.976 | 0 | 1.768 | 6.451 | 0 | 0 |
| SKNSH2000819 | 2.489 | 0 | 0 | 0 | 0 | 0 | 8.280 |
| SKNSH2001222 | 0 | 0 | 0 | 0 | 24.304 | 23.545 | 0 |
| SKNSH2001875 | 6.493 | 0 | 0 | 0 | 0 | 0 | 0 |
| SKNSH2002325 | 3.817 | 8.758 | 0 | 0 | 0 | 0 | 0 |
| SKNSH2003064 | 0 | 17.960 | 0 | 0 | 0 | 0 | 0 |
| SKNSH2005061 | 1.695 | 0 | 0 | 5.189 | 0 | 0 | 1.880 |
| SKNSH2009357 | 0 | 0 | 0 | 0 | 0 | 0 | 10.594 |
| SMINT1000048 | 7.876 | 0 | 0 | 8.036 | 0 | 0 | 0 |
| SMINT1000100 | 0 | 6.387 | 0 | 0 | 0 | 0 | 0 |
| SMINT1000117 | 7.892 | 0 | 0 | 4.026 | 0 | 0 | 2.917 |
| SMINT1000131 | 0 | 0 | 0 | 0 | 0 | 0 | 1.016 |
| SMINT2000073 | 0 | 0 | 0 | 0 | 0 | 0 | 9.621 |
| SMINT2000239 | 0 | 0 | 0 | 0 | 0 | 30.243 | 0 |
| SMINT2000267 | 0 | 0 | 0 | 0 | 0 | 0 | 23.912 |
| SMINT2000396 | 0 | 0 | 0 | 0 | 0 | 0 | 1.357 |
| SMINT2000400 | 6.689 | 0 | 0 | 0 | 0 | 0 | 0 |
| SMINT2000441 | 0.451 | 1.036 | 1.599 | 1.381 | 1.680 | 0 | 3.504 |
| SMINT2000454 | 0 | 20.504 | 0 | 0 | 0 | 0 | 9.911 |
| SMINT2000468 | 9.152 | 0 | 0 | 9.337 | 0 | 0 | 5.074 |
| SMINT2000545 | 2.371 | 0 | 0 | 2.419 | 0 | 0 | 0 |
| SMINT2000629 | 2.686 | 0 | 0 | 5.480 | 9.996 | 9.683 | 0 |
| SMINT2000659 | 0 | 0.637 | 0 | 0 | 0 | 0 | 0.616 |
| SMINT2000694 | 0 | 2.727 | 0 | 0 | 0 | 0 | 0 |
| SMINT2000933 | 0 | 0 | 0 | 0 | 0 | 3.675 | 1.130 |
| SMINT2001183 | 0 | 0 | 0 | 0 | 0 | 0 | 20.139 |
| SMINT2001348 | 0 | 0 | 32.864 | 0 | 0 | 0 | 0 |
| SMINT2001950 | 0 | 0 | 0 | 0 | 0 | 0 | 11.849 |
| SMINT2002159 | 0 | 0 | 0 | 6.995 | 0 | 0 | 0 |
| SMINT2002224 | 0 | 29.044 | 0 | 0 | 0 | 0 | 0 |
| SMINT2002620 | 0 | 0 | 8.026 | 9.246 | 0 | 0 | 2.512 |
| SMINT2002689 | 4.702 | 0 | 0 | 0 | 0 | 0 | 1.304 |
| SMINT2002976 | 10.432 | 0 | 0 | 5.322 | 0 | 0 | 0 |
| SMINT2003169 | 0.470 | 0 | 0 | 0.480 | 0 | 0 | 0 |
| SMINT2003423 | 0 | 0 | 0 | 0 | 40.436 | 0 | 0 |
| SMINT2003905 | 0.774 | 1.776 | 0 | 0 | 0 | 0 | 0.859 |
| SMINT2004023 | 0 | 0 | 5.403 | 0 | 0 | 0 | 0 |
| SMINT2004350 | 13.067 | 0 | 0 | 0 | 0 | 0 | 14.491 |
| SMINT2004589 | 0 | 0 | 0 | 4.089 | 0 | 0 | 0 |
| SMINT2004872 | 0 | 4.852 | 7.492 | 0 | 0 | 0 | 0 |
| SMINT2005330 | 0 | 0 | 0 | 0 | 51.332 | 0 | 0 |
| SMINT2005368 | 0 | 0 | 0 | 2.396 | 4.371 | 4.234 | 1.302 |
| SMINT2005956 | 0 | 0 | 0 | 2.685 | 0 | 0 | 17.513 |
| SMINT2007101 | 0 | 0 | 0 | 0 | 0 | 0 | 23.912 |
| SMINT2007792 | 0 | 0 | 29.610 | 0 | 0 | 0 | 9.269 |
| SMINT2008590 | 0 | 0 | 0 | 9.766 | 0 | 0 | 10.615 |
| SMINT2008869 | 0 | 0 | 50.098 | 0 | 0 | 0 | 0 |
| SMINT2008921 | 1.110 | 0 | 0 | 1.132 | 8.259 | 0 | 1.230 |
| SMINT2008960 | 1.368 | 4.709 | 2.424 | 0.698 | 5.093 | 12.334 | 0.759 |
| SMINT2009292 | 0 | 8.138 | 12.565 | 0 | 13.202 | 0 | 3.933 |
| SMINT2009415 | 0 | 0 | 0 | 0 | 0 | 0 | 23.912 |
| SMINT2010084 | 6.872 | 7.883 | 0 | 7.011 | 12.789 | 12.389 | 0 |
| SMINT2010144 | 0 | 0 | 0 | 0 | 0 | 0 | 23.912 |
| SMINT2010324 | 0 | 0 | 0 | 0 | 0 | 0 | 23.912 |
| SMINT2010639 | 2.709 | 18.644 | 0 | 0 | 0 | 29.302 | 0 |
| SMINT2010901 | 0 | 0 | 0 | 0 | 13.071 | 0 | 3.895 |
| SMINT2012122 | 0 | 0 | 12.384 | 0 | 0 | 0 | 3.877 |
| SMINT2012179 | 0 | 0 | 0 | 18.954 | 0 | 0 | 0 |
| SMINT2012501 | 0 | 0 | 0 | 8.113 | 0 | 0 | 0 |
| SMINT2013031 | 0 | 0 | 0 | 3.166 | 0 | 0 | 0 |
| SMINT2014480 | 0 | 0 | 5.628 | 0 | 5.913 | 0 | 0 |
| SMINT2016146 | 0 | 39.399 | 0 | 0 | 0 | 0 | 0 |
| SMINT2016396 | 0 | 0 | 50.098 | 0 | 0 | 0 | 0 |
| SMINT2017134 | 0 | 0 | 0 | 0 | 0 | 0 | 23.912 |
| SPLEN1000013 | 0 | 0 | 0 | 9.928 | 0 | 0 | 0 |
| SPLEN1000049 | 21.800 | 0 | 0 | 0 | 0 | 0 | 0 |
| SPLEN1000106 | 2.328 | 0 | 0 | 0 | 0 | 0 | 0 |
| SPLEN1000116 | 0.990 | 0 | 0 | 0 | 0 | 0 | 0 |
| SPLEN1000144 | 0 | 0 | 0 | 5.333 | 0 | 0 | 0 |
| SPLEN1000165 | 4.990 | 0 | 0 | 0 | 0 | 0 | 0 |
| SPLEN1000166 | 3.391 | 0 | 0 | 3.460 | 0 | 0 | 2.507 |
| SPLEN2000098 | 12.045 | 0 | 0 | 0 | 0 | 0 | 0 |
| SPLEN2000134 | 0 | 12.019 | 0 | 0 | 0 | 0 | 5.810 |
| SPLEN2000197 | 0 | 0 | 0 | 0 | 0 | 0 | 38.930 |
| SPLEN2000222 | 12.214 | 0 | 0 | 0 | 0 | 0 | 0 |
| SPLEN2000255 | 3.071 | 0 | 5.441 | 0 | 5.716 | 0 | 0 |
| SPLEN2000258 | 0 | 0 | 0 | 0 | 0 | 0 | 9.710 |
| SPLEN2000267 | 17.514 | 0 | 0 | 5.956 | 0 | 0 | 6.474 |
| SPLEN2000341 | 0 | 0 | 0 | 0 | 0 | 0 | 15.016 |
| SPLEN2000348 | 2.797 | 12.836 | 0 | 0 | 0 | 0 | 0 |
| SPLEN2000505 | 0.421 | 0 | 1.493 | 1.290 | 1.569 | 1.520 | 2.337 |
| SPLEN2000537 | 36.501 | 0 | 0 | 0 | 0 | 0 | 0 |
| SPLEN2000698 | 0 | 0 | 0 | 2.163 | 7.892 | 0 | 2.351 |
| SPLEN2000874 | 0.487 | 0 | 0 | 0 | 0 | 0 | 0 |
| SPLEN2000882 | 0 | 0 | 0 | 0 | 0 | 0 | 1.948 |
| SPLEN2001141 | 0 | 0 | 0 | 0.963 | 0 | 0 | 2.094 |
| SPLEN2001157 | 2.751 | 0 | 0 | 0 | 0 | 0 | 3.050 |
| SPLEN2001626 | 0 | 0 | 0 | 0 | 0 | 67.455 | 0 |
| SPLEN2002053 | 36.501 | 0 | 0 | 0 | 0 | 0 | 0 |
| SPLEN2002272 | 0 | 0 | 0 | 0 | 0 | 67.455 | 0 |
| SPLEN2002335 | 2.025 | 0 | 0 | 3.099 | 0 | 0 | 3.369 |
| SPLEN2002463 | 7.573 | 0 | 0 | 30.903 | 0 | 0 | 8.398 |
| SPLEN2002493 | 13.846 | 0 | 0 | 3.532 | 0 | 0 | 19.194 |
| SPLEN2002744 | 2.230 | 0 | 7.901 | 0 | 0 | 0 | 0 |
| SPLEN2002931 | 4.078 | 0 | 0 | 0 | 0 | 0 | 0 |
| SPLEN2003396 | 0 | 0 | 0 | 0 | 0 | 67.455 | 0 |
| SPLEN2003678 | 0 | 0 | 0 | 0 | 0 | 0 | 38.930 |
| SPLEN2004343 | 35.982 | 0 | 0 | 0 | 0 | 0 | 19.951 |
| SPLEN2004555 | 4.258 | 0 | 0 | 1.086 | 0 | 0 | 3.541 |
| SPLEN2004773 | 2.847 | 0 | 0 | 2.178 | 10.596 | 0 | 2.368 |
| SPLEN2004880 | 0 | 0.676 | 0 | 0 | 0 | 0 | 0 |
| SPLEN2004984 | 0 | 0 | 9.305 | 0 | 0 | 0 | 2.913 |
| SPLEN2005009 | 8.597 | 0 | 0 | 0 | 0 | 0 | 19.067 |
| SPLEN2005342 | 0 | 0 | 0 | 5.443 | 0 | 0 | 0 |
| SPLEN2005429 | 0 | 0 | 0 | 3.738 | 0 | 0 | 4.063 |
| SPLEN2005560 | 0 | 0 | 3.729 | 0 | 0 | 0 | 0 |
| SPLEN2005783 | 19.557 | 14.956 | 0 | 0 | 0 | 0 | 7.229 |
| SPLEN2005806 | 0 | 0 | 0 | 0 | 0 | 4.410 | 0 |
| SPLEN2005838 | 0 | 0 | 0 | 36.967 | 0 | 0 | 0 |
| SPLEN2005927 | 0 | 9.879 | 0 | 0 | 0 | 0 | 0 |
| SPLEN2006122 | 0 | 0 | 0 | 0 | 0 | 0 | 0.934 |
| SPLEN2006133 | 1.897 | 0 | 0 | 2.904 | 0 | 0 | 0 |
| SPLEN2006143 | 2.237 | 0 | 0 | 2.283 | 8.327 | 0 | 2.481 |
| SPLEN2006305 | 0.632 | 1.449 | 2.238 | 0 | 0 | 0 | 0.700 |
| SPLEN2006374 | 6.080 | 0 | 0 | 0 | 5.657 | 10.961 | 3.371 |
| SPLEN2007182 | 0 | 0 | 0 | 0 | 47.571 | 0 | 0 |
| SPLEN2007276 | 0 | 6.084 | 9.395 | 0 | 9.871 | 0 | 0 |
| SPLEN2007498 | 5.234 | 0 | 0 | 10.680 | 0 | 0 | 2.902 |
| SPLEN2007739 | 12.144 | 0 | 0 | 0 | 0 | 0 | 26.935 |
| SPLEN2008460 | 0 | 0 | 0 | 2.673 | 0 | 0 | 0 |
| SPLEN2009081 | 0 | 0 | 22.928 | 0 | 0 | 0 | 0 |
| SPLEN2009555 | 0 | 0 | 0 | 0 | 0 | 0 | 9.419 |
| SPLEN2009970 | 0 | 0 | 0 | 5.203 | 0 | 0 | 0 |
| SPLEN2010015 | 0 | 0 | 0 | 31.720 | 0 | 0 | 17.239 |
| SPLEN2010195 | 6.207 | 0 | 0 | 3.562 | 0 | 0 | 3.012 |
| SPLEN2010469 | 0 | 56.874 | 0 | 0 | 0 | 0 | 0 |
| SPLEN2010912 | 0 | 0 | 0 | 12.636 | 0 | 0 | 13.734 |
| SPLEN2011419 | 0 | 0 | 0 | 2.438 | 0 | 8.616 | 0 |
| SPLEN2011678 | 0 | 0 | 0 | 0 | 0 | 0 | 1.264 |
| SPLEN2011737 | 5.211 | 5.977 | 0 | 10.632 | 0 | 9.394 | 0 |
| SPLEN2012523 | 0 | 0 | 0 | 0 | 0 | 0 | 1.184 |
| SPLEN2012571 | 0 | 0 | 11.262 | 0 | 0 | 0 | 0 |
| SPLEN2012619 | 36.501 | 0 | 0 | 0 | 0 | 0 | 0 |
| SPLEN2012624 | 0 | 0 | 0 | 10.317 | 0 | 0 | 0 |
| SPLEN2012800 | 0 | 0.784 | 0 | 0 | 0 | 0 | 0.379 |
| SPLEN2013503 | 0 | 5.758 | 0 | 0 | 0 | 0 | 0 |
| SPLEN2013857 | 36.501 | 0 | 0 | 0 | 0 | 0 | 0 |
| SPLEN2013936 | 0 | 0 | 0 | 0 | 0 | 0 | 7.399 |
| SPLEN2014119 | 0 | 0 | 59.122 | 0 | 0 | 0 | 0 |
| SPLEN2014572 | 0 | 0 | 22.047 | 0 | 0 | 0 | 6.902 |
| SPLEN2014645 | 0 | 0 | 0 | 0 | 68.148 | 0 | 0 |
| SPLEN2014711 | 2.652 | 0 | 0 | 0 | 0 | 0 | 0 |
| SPLEN2014911 | 0 | 0 | 0 | 0 | 0 | 0 | 2.340 |
| SPLEN2015129 | 36.501 | 0 | 0 | 0 | 0 | 0 | 0 |
| SPLEN2015310 | 1.146 | 2.630 | 2.030 | 0 | 0 | 0 | 1.271 |
| SPLEN2015788 | 0 | 0 | 0 | 0 | 0 | 0 | 5.368 |
| SPLEN2016139 | 1.281 | 0 | 0 | 0 | 0 | 0 | 1.421 |
| SPLEN2016268 | 0 | 0 | 0 | 0 | 0 | 1.558 | 0.479 |
| SPLEN2016421 | 0 | 0 | 0 | 0 | 0 | 0 | 9.189 |
| SPLEN2016533 | 0 | 0 | 0 | 0 | 0 | 0 | 38.930 |
| SPLEN2016743 | 5.733 | 0 | 0 | 0 | 0 | 0 | 0 |
| SPLEN2016932 | 0 | 0 | 0 | 0 | 0 | 0 | 38.930 |
| SPLEN2016972 | 0 | 0 | 0 | 2.069 | 0 | 0 | 0 |
| SPLEN2017121 | 7.753 | 0 | 0 | 0 | 0 | 0 | 8.598 |
| SPLEN2017318 | 5.074 | 0 | 0 | 0 | 0 | 0 | 11.254 |
| SPLEN2017351 | 9.023 | 0 | 0 | 0 | 11.195 | 0 | 6.671 |
| SPLEN2017918 | 0 | 0 | 3.230 | 0 | 0 | 0 | 0 |
| SPLEN2018157 | 0 | 0 | 0 | 2.461 | 0 | 0 | 2.675 |
| SPLEN2018159 | 0 | 0 | 0 | 0 | 0 | 0 | 10.502 |
| SPLEN2018299 | 41.154 | 0 | 0 | 0 | 0 | 0 | 0 |
| SPLEN2018302 | 0 | 0 | 0 | 6.204 | 0 | 0 | 0 |
| SPLEN2018643 | 36.501 | 0 | 0 | 0 | 0 | 0 | 0 |
| SPLEN2019169 | 5.661 | 0 | 0 | 0 | 0 | 0 | 0 |
| SPLEN2019349 | 0 | 0 | 0 | 36.967 | 0 | 0 | 0 |
| SPLEN2019405 | 1.432 | 6.571 | 5.073 | 1.461 | 0 | 0 | 1.588 |
| SPLEN2019774 | 0 | 0 | 0 | 0 | 33.067 | 0 | 0 |
| SPLEN2020034 | 0 | 56.874 | 0 | 0 | 0 | 0 | 0 |
| SPLEN2020417 | 0 | 0 | 0 | 0 | 0 | 50.331 | 15.479 |
| SPLEN2021122 | 0 | 0 | 0 | 33.279 | 0 | 0 | 18.087 |
| SPLEN2021194 | 3.373 | 0 | 0 | 0 | 0 | 0 | 0 |
| SPLEN2021273 | 14.747 | 0 | 0 | 0 | 0 | 0 | 16.354 |
| SPLEN2021458 | 0 | 0 | 0 | 0 | 0 | 0 | 38.930 |
| SPLEN2023118 | 0 | 0 | 39.433 | 0 | 0 | 0 | 0 |
| SPLEN2023160 | 0 | 0 | 0 | 40.108 | 0 | 0 | 0 |
| SPLEN2024489 | 0 | 0 | 38.706 | 22.294 | 0 | 0 | 0 |
| SPLEN2024922 | 0 | 0 | 0 | 26.295 | 0 | 0 | 0 |
| SPLEN2025491 | 0 | 0 | 0 | 0 | 0 | 0 | 38.930 |
| SPLEN2027113 | 1.636 | 0 | 0 | 0 | 0 | 0 | 1.815 |
| SPLEN2028466 | 0.414 | 0 | 4.396 | 4.642 | 6.159 | 5.966 | 3.211 |
| SPLEN2028914 | 2.792 | 0 | 0 | 0 | 0 | 0 | 0 |
| SPLEN2031424 | 15.229 | 0 | 0 | 0 | 0 | 0 | 8.444 |
| SPLEN2031547 | 1.574 | 0 | 0 | 11.244 | 0 | 0 | 1.746 |
| SPLEN2031674 | 0 | 0 | 0 | 0 | 0 | 0 | 24.555 |
| SPLEN2032154 | 0 | 0 | 0 | 0 | 0 | 0 | 38.930 |
| SPLEN2034448 | 5.185 | 0 | 0 | 5.290 | 0 | 0 | 0 |
| SPLEN2034781 | 27.984 | 0 | 0 | 0 | 0 | 0 | 0 |
| SPLEN2036076 | 0 | 0 | 0 | 1.676 | 0 | 0 | 0 |
| SPLEN2036821 | 0 | 0 | 0 | 0 | 0 | 0 | 38.930 |
| SPLEN2036932 | 2.932 | 6.726 | 31.158 | 5.982 | 0 | 0 | 0 |
| SPLEN2039379 | 5.792 | 0 | 0 | 8.864 | 0 | 0 | 3.212 |
| SPLEN2040824 | 41.249 | 0 | 0 | 0 | 0 | 0 | 22.872 |
| SPLEN2042535 | 0 | 0 | 0 | 7.264 | 0 | 0 | 0 |
| STOMA1000091 | 0 | 0 | 0 | 0 | 0 | 0 | 7.714 |
| STOMA2000257 | 0 | 0 | 0 | 0 | 7.347 | 0 | 0 |
| STOMA2000289 | 0 | 6.250 | 9.651 | 0 | 10.140 | 0 | 0 |
| STOMA2000396 | 0 | 0 | 0 | 5.258 | 0 | 0 | 5.715 |
| STOMA2000482 | 0 | 0 | 0 | 0 | 0 | 0 | 4.275 |
| STOMA2002052 | 5.562 | 0 | 0 | 5.674 | 0 | 0 | 0 |
| STOMA2002166 | 0 | 0 | 0 | 0 | 0 | 2.109 | 0 |
| STOMA2005120 | 0 | 0 | 0 | 0 | 0 | 0 | 14.012 |
| STOMA2006229 | 6.012 | 0 | 0 | 0 | 0 | 0 | 0 |
| STOMA2009289 | 5.737 | 0 | 0 | 0 | 0 | 0 | 0 |
| SYNOV1000124 | 5.006 | 1.276 | 0 | 6.810 | 6.211 | 0 | 0.617 |
| SYNOV1000128 | 0 | 0 | 0 | 0 | 0 | 0 | 16.279 |
| SYNOV1000374 | 0 | 0 | 0 | 0 | 0 | 0 | 22.005 |
| SYNOV2000291 | 0 | 0 | 0 | 32.112 | 0 | 0 | 0 |
| SYNOV2000397 | 0 | 0 | 0 | 48.614 | 0 | 0 | 0 |
| SYNOV2001033 | 0 | 0 | 0 | 0.612 | 0 | 2.162 | 0 |
| SYNOV2001144 | 0 | 3.245 | 0 | 0 | 5.264 | 0 | 3.137 |
| SYNOV2001239 | 0 | 0 | 1.112 | 0 | 0 | 0 | 1.044 |
| SYNOV2001262 | 2.086 | 4.787 | 0 | 0 | 0 | 7.523 | 4.627 |
| SYNOV2001581 | 0 | 6.042 | 0 | 0 | 0 | 0 | 0 |
| SYNOV2001648 | 0 | 0 | 0 | 0 | 0 | 0 | 1.202 |
| SYNOV2006620 | 0 | 0 | 0 | 0 | 0 | 0 | 33.957 |
| SYNOV2007758 | 19.566 | 0 | 0 | 0 | 0 | 0 | 0 |
| SYNOV4000249 | 0 | 0 | 0 | 48.614 | 0 | 0 | 0 |
| SYNOV4001153 | 2.319 | 0 | 0 | 26.029 | 0 | 0 | 7.716 |
| SYNOV4002883 | 23.940 | 0 | 0 | 24.425 | 0 | 0 | 0 |
| SYNOV4005989 | 0 | 0 | 0 | 21.059 | 0 | 0 | 0 |
| SYNOV4006542 | 0 | 0 | 0 | 3.782 | 0 | 0 | 4.111 |
| SYNOV4007430 | 31.677 | 0 | 0 | 0 | 0 | 0 | 0 |
| SYNOV4007671 | 0 | 0 | 0 | 0 | 0 | 0 | 0.813 |
| SYNOV4008245 | 9.239 | 0 | 0 | 28.277 | 0 | 0 | 10.245 |
| SYNOV4009129 | 7.763 | 17.811 | 0 | 0 | 0 | 0 | 0 |
| SYNOV4009575 | 0 | 0 | 0 | 0 | 0 | 17.276 | 0 |
| T1ESE2000904 | 0 | 0 | 0 | 0 | 8.593 | 0 | 0 |
| TBAES2007428 | 5.371 | 0 | 19.027 | 0 | 0 | 0 | 0 |
| TBAES2009387 | 0 | 0 | 1.894 | 0 | 0 | 0 | 0.593 |
| TCOLN1000180 | 0 | 0 | 0 | 0 | 0 | 8.828 | 0 |
| TESOP1000035 | 0 | 10.147 | 0 | 0 | 0 | 15.948 | 4.905 |
| TESOP2000527 | 0 | 0 | 0 | 0 | 0 | 0 | 11.444 |
| TESOP2002451 | 2.375 | 0 | 0 | 0 | 0 | 0 | 0 |
| TESOP2006893 | 0 | 0 | 0 | 0 | 0 | 30.976 | 0 |
| TESTI1000023 | 15.934 | 1.924 | 0 | 7.700 | 0 | 3.024 | 3.720 |
| TESTI1000051 | 0 | 0 | 0 | 10.627 | 0 | 0 | 0 |
| TESTI1000070 | 0 | 0 | 0 | 3.684 | 0 | 0 | 0 |
| TESTI1000094 | 0 | 22.629 | 0 | 0 | 0 | 0 | 0 |
| TESTI1000096 | 0 | 0 | 0 | 0 | 0 | 0 | 19.400 |
| TESTI1000101 | 0 | 9.827 | 0 | 8.740 | 0 | 0 | 0 |
| TESTI1000163 | 4.120 | 0 | 0 | 0 | 0 | 0 | 0 |
| TESTI1000168 | 0 | 0 | 11.102 | 0 | 0 | 0 | 0 |
| TESTI2000006 | 0 | 0 | 0 | 0 | 0 | 0 | 10.966 |
| TESTI2000172 | 0 | 0 | 0 | 0 | 0 | 0 | 9.487 |
| TESTI2000177 | 0 | 0 | 0 | 24.875 | 0 | 0 | 0 |
| TESTI2000184 | 4.522 | 0 | 0 | 3.460 | 0 | 4.076 | 3.761 |
| TESTI2000435 | 19.231 | 0 | 0 | 0 | 0 | 0 | 0 |
| TESTI2000489 | 0 | 0 | 0 | 0 | 0 | 57.966 | 0 |
| TESTI2000520 | 0 | 0 | 0 | 0 | 0 | 13.565 | 0 |
| TESTI2000600 | 0 | 0 | 0 | 75.737 | 0 | 0 | 0 |
| TESTI2000627 | 0 | 0 | 0 | 21.496 | 0 | 0 | 0 |
| TESTI2000784 | 2.273 | 0 | 0 | 2.319 | 0 | 0 | 0 |
| TESTI2000849 | 0 | 0 | 0 | 0 | 0 | 0 | 29.781 |
| TESTI2000874 | 0 | 0 | 0 | 0 | 46.526 | 0 | 0 |
| TESTI2000883 | 7.291 | 0 | 25.830 | 0 | 0 | 52.580 | 0 |
| TESTI2000970 | 0 | 0 | 17.221 | 0 | 18.093 | 0 | 0 |
| TESTI2001099 | 1.470 | 1.124 | 0 | 1.500 | 0 | 0 | 1.087 |
| TESTI2001120 | 0 | 0 | 0 | 0.986 | 0 | 0 | 0 |
| TESTI2001141 | 0 | 0 | 0 | 9.634 | 0 | 0 | 0 |
| TESTI2001153 | 0.398 | 0 | 1.411 | 0 | 0 | 0 | 0 |
| TESTI2001269 | 2.183 | 0 | 0 | 2.785 | 0 | 0 | 2.421 |
| TESTI2001613 | 13.661 | 0 | 0 | 41.812 | 0 | 0 | 0 |
| TESTI2001829 | 0 | 1.948 | 0 | 0 | 0 | 0 | 0.942 |
| TESTI2001862 | 0 | 6.066 | 0 | 0 | 0 | 9.534 | 2.932 |
| TESTI2001869 | 0 | 54.752 | 0 | 0 | 0 | 0 | 0 |
| TESTI2001898 | 0 | 0 | 0 | 60.949 | 0 | 0 | 0 |
| TESTI2001985 | 5.870 | 0 | 0 | 0 | 0 | 0 | 0 |
| TESTI2002256 | 0 | 0 | 0 | 0 | 0 | 0 | 38.468 |
| TESTI2002365 | 0 | 2.505 | 0 | 0 | 0 | 0 | 2.422 |
| TESTI2002467 | 0 | 0 | 0 | 0 | 0 | 84.653 | 0 |
| TESTI2002632 | 0 | 0 | 0 | 0 | 40.298 | 0 | 0 |
| TESTI2002707 | 13.255 | 7.603 | 17.609 | 11.833 | 6.167 | 0 | 7.350 |
| TESTI2003141 | 0 | 0 | 0 | 0 | 0 | 40.686 | 0 |
| TESTI2003280 | 0 | 18.822 | 0 | 0 | 0 | 0 | 0 |
| TESTI2003413 | 2.604 | 0 | 9.224 | 0 | 0 | 0 | 2.887 |
| TESTI2003454 | 0 | 0 | 0 | 5.418 | 0 | 0 | 0 |
| TESTI2003533 | 0.867 | 1.989 | 0 | 0 | 0 | 0 | 0.961 |
| TESTI2003541 | 0 | 0 | 0 | 0 | 0 | 0 | 32.389 |
| TESTI2003596 | 0 | 0 | 14.759 | 0 | 0 | 0 | 0 |
| TESTI2003625 | 0 | 0 | 0 | 0 | 28.488 | 0 | 0 |
| TESTI2003692 | 0 | 0 | 0 | 60.949 | 0 | 0 | 0 |
| TESTI2003756 | 0 | 0 | 0 | 0 | 0 | 0 | 11.634 |
| TESTI2004031 | 0 | 0 | 0 | 0 | 1.492 | 0 | 0 |
| TESTI2004080 | 0 | 0 | 0 | 0 | 0 | 0 | 10.824 |
| TESTI2004153 | 0 | 0 | 0 | 4.426 | 0 | 15.643 | 0 |
| TESTI2004295 | 8.188 | 0 | 0 | 8.354 | 0 | 0 | 0 |
| TESTI2004431 | 8.365 | 0 | 0 | 0 | 0 | 0 | 0 |
| TESTI2004734 | 0 | 0 | 0 | 34.985 | 0 | 0 | 0 |
| TESTI2004737 | 1.771 | 0 | 6.275 | 3.614 | 0 | 0 | 1.964 |
| TESTI2004929 | 0 | 6.514 | 0 | 0 | 0 | 0 | 0 |
| TESTI2005112 | 19.455 | 0 | 0 | 0 | 0 | 0 | 0 |
| TESTI2005408 | 0 | 4.065 | 6.276 | 3.615 | 6.594 | 0 | 0 |
| TESTI2005564 | 0 | 8.402 | 0 | 3.736 | 0 | 0 | 0 |
| TESTI2005731 | 0 | 0 | 0 | 3.371 | 0 | 0 | 0 |
| TESTI2005742 | 40.787 | 0 | 0 | 0 | 0 | 0 | 0 |
| TESTI2005743 | 0 | 5.040 | 0 | 0 | 0 | 0 | 0 |
| TESTI2005759 | 0 | 0 | 3.313 | 0 | 0 | 0 | 0 |
| TESTI2005784 | 5.949 | 6.824 | 0 | 21.241 | 0 | 0 | 9.895 |
| TESTI2005788 | 0 | 0 | 0 | 4.718 | 0 | 0 | 0 |
| TESTI2005835 | 0 | 0 | 0 | 0 | 0 | 0 | 13.362 |
| TESTI2005908 | 0 | 3.613 | 5.578 | 0 | 0 | 0 | 0 |
| TESTI2006051 | 0 | 0 | 0 | 0 | 1.576 | 1.527 | 0 |
| TESTI2006111 | 0 | 47.300 | 0 | 0 | 0 | 0 | 0 |
| TESTI2006258 | 10.254 | 0 | 36.326 | 0 | 38.166 | 0 | 0 |
| TESTI2006383 | 0 | 0 | 0 | 4.135 | 0 | 0 | 0 |
| TESTI2006409 | 0 | 0 | 0 | 60.949 | 0 | 0 | 0 |
| TESTI2006453 | 1.683 | 3.861 | 0 | 0.859 | 0 | 0 | 2.800 |
| TESTI2006588 | 1.083 | 1.242 | 1.918 | 1.105 | 0 | 1.952 | 1.201 |
| TESTI2006748 | 0 | 7.892 | 0 | 0 | 0 | 0 | 0 |
| TESTI2007211 | 0 | 0 | 0 | 0 | 0 | 0 | 3.663 |
| TESTI2007311 | 0 | 0 | 0 | 3.925 | 0 | 41.615 | 4.266 |
| TESTI2007407 | 0 | 0 | 0 | 0 | 0 | 0 | 3.404 |
| TESTI2007998 | 1.581 | 10.883 | 0 | 1.613 | 0 | 0 | 1.753 |
| TESTI2008020 | 3.835 | 0 | 0 | 0 | 0 | 0 | 0 |
| TESTI2008240 | 1.500 | 0.574 | 0 | 1.531 | 0.931 | 6.312 | 0 |
| TESTI2008621 | 0 | 0 | 35.791 | 0 | 0 | 0 | 0 |
| TESTI2009520 | 4.500 | 0 | 2.657 | 3.826 | 5.583 | 5.408 | 0.832 |
| TESTI2009577 | 0 | 0 | 0 | 1.803 | 0 | 0 | 1.960 |
| TESTI2009785 | 1.435 | 0 | 5.083 | 1.464 | 0 | 0 | 0 |
| TESTI2009987 | 10.202 | 0 | 0 | 5.204 | 0 | 0 | 0 |
| TESTI2010180 | 0 | 0 | 0 | 0 | 0 | 0 | 38.468 |
| TESTI2010513 | 7.465 | 0.901 | 0 | 8.017 | 0 | 0 | 2.178 |
| TESTI2010617 | 6.964 | 0 | 0 | 21.314 | 0 | 0 | 0 |
| TESTI2011605 | 0 | 0 | 0 | 0 | 0 | 0 | 3.997 |
| TESTI2011680 | 7.481 | 0 | 0 | 0 | 0 | 0 | 0 |
| TESTI2012155 | 6.408 | 0 | 0 | 6.538 | 0 | 0 | 7.106 |
| TESTI2012571 | 0 | 0 | 36.286 | 0 | 0 | 36.932 | 0 |
| TESTI2013231 | 1.990 | 0 | 0 | 0 | 0 | 0 | 0 |
| TESTI2013295 | 2.664 | 0 | 0 | 0 | 0 | 0 | 0 |
| TESTI2014780 | 0 | 0 | 0 | 28.973 | 0 | 0 | 0 |
| TESTI2014838 | 3.975 | 0 | 0 | 0 | 0 | 0 | 1.469 |
| TESTI2015042 | 0 | 0 | 0 | 43.832 | 0 | 0 | 0 |
| TESTI2015092 | 0 | 0 | 11.736 | 0 | 12.331 | 0 | 7.348 |
| TESTI2015213 | 0 | 0 | 1.818 | 0.524 | 0 | 0 | 0 |
| TESTI2015626 | 0 | 0.754 | 1.165 | 0.336 | 1.224 | 2.372 | 0 |
| TESTI2015962 | 11.900 | 9.101 | 0 | 8.094 | 14.764 | 0 | 8.798 |
| TESTI2016278 | 0 | 0 | 0 | 0 | 0 | 0 | 62.915 |
| TESTI2016583 | 0 | 0 | 63.719 | 0 | 0 | 0 | 0 |
| TESTI2016896 | 0 | 0 | 0 | 43.832 | 0 | 0 | 0 |
| TESTI2017035 | 0 | 0 | 0 | 42.801 | 0 | 0 | 0 |
| TESTI2017645 | 0 | 0 | 0 | 0 | 0 | 0 | 36.905 |
| TESTI2017954 | 0.662 | 0 | 0 | 0 | 0 | 0 | 0.735 |
| TESTI2018014 | 7.709 | 0 | 0 | 0 | 0 | 0 | 0 |
| TESTI2018421 | 0 | 0 | 0 | 0 | 0 | 84.653 | 0 |
| TESTI2018462 | 2.826 | 0 | 0 | 0 | 0 | 0 | 9.401 |
| TESTI2018611 | 0 | 2.410 | 0 | 0 | 0 | 0 | 0 |
| TESTI2018997 | 0 | 0 | 7.378 | 0 | 0 | 0 | 0 |
| TESTI2020012 | 0 | 0 | 0 | 0 | 85.061 | 0 | 0 |
| TESTI2020956 | 0 | 0 | 0 | 0 | 0 | 0 | 4.772 |
| TESTI2021038 | 42.311 | 0 | 0 | 0 | 0 | 0 | 0 |
| TESTI2021124 | 17.146 | 0 | 0 | 11.662 | 0 | 0 | 0 |
| TESTI2021297 | 0 | 0 | 0 | 0 | 0 | 0 | 7.613 |
| TESTI2021358 | 0 | 0 | 0 | 0 | 0 | 0 | 37.524 |
| TESTI2021425 | 1.722 | 0 | 0 | 0 | 0 | 0 | 1.909 |
| TESTI2021531 | 0 | 0 | 0 | 0 | 0 | 60.329 | 0 |
| TESTI2021654 | 9.773 | 0 | 0 | 0 | 0 | 0 | 10.838 |
| TESTI2022179 | 0 | 24.096 | 0 | 0 | 0 | 0 | 0 |
| TESTI2023033 | 0 | 0 | 0 | 0 | 0 | 0 | 15.227 |
| TESTI2024598 | 0 | 0 | 0 | 60.949 | 0 | 0 | 0 |
| TESTI2025013 | 0 | 0 | 0 | 0 | 0 | 12.834 | 0 |
| TESTI2025454 | 0 | 0 | 0 | 0 | 0 | 0 | 12.490 |
| TESTI2025757 | 33.912 | 0 | 0 | 0 | 0 | 0 | 0 |
| TESTI2025846 | 0 | 0 | 0 | 20.770 | 0 | 0 | 22.576 |
| TESTI2026116 | 0 | 34.218 | 0 | 0 | 0 | 0 | 0 |
| TESTI2026215 | 6.927 | 0 | 0 | 7.067 | 0 | 0 | 0 |
| TESTI2026525 | 0 | 0 | 0 | 0 | 16.193 | 0 | 0 |
| TESTI2026534 | 0 | 0 | 0 | 60.949 | 0 | 0 | 0 |
| TESTI2027496 | 0 | 0 | 33.283 | 9.585 | 0 | 0 | 10.419 |
| TESTI2028296 | 0 | 0 | 0 | 2.516 | 0 | 8.891 | 0 |
| TESTI2029227 | 0 | 0 | 0 | 6.056 | 0 | 21.404 | 6.583 |
| TESTI2029259 | 6.619 | 0 | 0 | 3.377 | 0 | 0 | 3.670 |
| TESTI2033031 | 0 | 0 | 0 | 0 | 0 | 0 | 34.120 |
| TESTI2033441 | 0 | 0 | 0 | 0 | 0 | 0 | 2.434 |
| TESTI2034916 | 7.602 | 0 | 0 | 0 | 0 | 0 | 0 |
| TESTI2034940 | 0 | 2.391 | 0 | 0 | 0 | 0.939 | 0 |
| TESTI2035236 | 0 | 0 | 0 | 0 | 0 | 0 | 57.175 |
| TESTI2035309 | 0 | 0 | 0 | 0 | 0 | 73.390 | 0 |
| TESTI2036691 | 0 | 0 | 0 | 0 | 0 | 84.653 | 0 |
| TESTI2036969 | 7.186 | 0 | 0 | 3.666 | 0 | 0 | 3.984 |
| TESTI2039177 | 16.578 | 0 | 0 | 21.141 | 0 | 0 | 18.384 |
| TESTI2039342 | 0 | 0 | 0 | 0 | 0 | 0 | 77.236 |
| TESTI2044276 | 0 | 10.879 | 0 | 0 | 0 | 0 | 10.517 |
| TESTI2045562 | 0 | 0 | 0 | 0 | 0 | 84.653 | 0 |
| TESTI2046456 | 0 | 1.062 | 0 | 0.944 | 0 | 1.669 | 1.540 |
| TESTI2049246 | 0 | 0 | 0 | 53.653 | 0 | 0 | 29.159 |
| TESTI2053723 | 0 | 0 | 0 | 4.619 | 0 | 0 | 10.040 |
| TESTI4000014 | 1.964 | 0 | 0 | 2.551 | 0.665 | 0.644 | 1.188 |
| TESTI4000214 | 0 | 0 | 0 | 7.979 | 0 | 0 | 0 |
| TESTI4001467 | 0 | 0 | 0 | 0 | 0 | 0 | 6.140 |
| TESTI4001569 | 0 | 0 | 0 | 43.832 | 0 | 0 | 0 |
| TESTI4001604 | 0 | 0 | 0 | 29.688 | 0 | 0 | 32.269 |
| TESTI4001984 | 60.471 | 0 | 0 | 0 | 0 | 0 | 0 |
| TESTI4002072 | 16.696 | 0 | 0 | 20.440 | 0 | 0 | 11.109 |
| TESTI4002195 | 0 | 0 | 23.701 | 0 | 0 | 0 | 0 |
| TESTI4002202 | 9.946 | 0 | 11.744 | 0 | 0 | 0 | 0 |
| TESTI4002290 | 0 | 0 | 0 | 0 | 0 | 0 | 62.915 |
| TESTI4002799 | 1.767 | 0 | 0 | 2.703 | 0 | 1.592 | 1.469 |
| TESTI4003150 | 14.850 | 0 | 0 | 15.151 | 0 | 0 | 0 |
| TESTI4003602 | 25.967 | 0 | 0 | 0 | 0 | 0 | 0 |
| TESTI4003703 | 0 | 0 | 0 | 4.514 | 0 | 0 | 4.907 |
| TESTI4003944 | 0 | 0 | 0 | 35.590 | 0 | 0 | 0 |
| TESTI4004210 | 36.051 | 0 | 0 | 0 | 0 | 0 | 0 |
| TESTI4004626 | 0 | 0 | 0 | 0 | 0 | 0 | 36.513 |
| TESTI4004695 | 0 | 0 | 0 | 0 | 0 | 0 | 36.122 |
| TESTI4005133 | 11.925 | 0 | 0 | 0 | 0 | 0 | 13.224 |
| TESTI4005399 | 7.797 | 0 | 0 | 15.910 | 0 | 0 | 0 |
| TESTI4006137 | 25.755 | 0 | 0 | 0 | 0 | 0 | 28.561 |
| TESTI4006407 | 0 | 0 | 0 | 0 | 0 | 0 | 62.915 |
| TESTI4006441 | 60.471 | 0 | 0 | 0 | 0 | 0 | 0 |
| TESTI4007965 | 0 | 0 | 0 | 0 | 0 | 0 | 20.014 |
| TESTI4008058 | 8.814 | 20.221 | 0 | 0 | 0 | 31.781 | 0 |
| TESTI4008097 | 60.471 | 0 | 0 | 0 | 0 | 0 | 0 |
| TESTI4008797 | 12.429 | 0 | 0 | 12.681 | 0 | 0 | 27.567 |
| TESTI4009286 | 1.450 | 0 | 0 | 1.479 | 0 | 0 | 0.804 |
| TESTI4009454 | 8.038 | 0 | 0 | 0 | 0 | 0 | 8.914 |
| TESTI4010377 | 0 | 0 | 0 | 60.949 | 0 | 0 | 0 |
| TESTI4010851 | 3.280 | 0 | 0 | 3.904 | 2.035 | 0 | 3.031 |
| TESTI4010928 | 0 | 0 | 0 | 34.221 | 0 | 0 | 0 |
| TESTI4010979 | 0 | 0 | 0 | 0 | 0 | 0 | 21.790 |
| TESTI4011084 | 0 | 0 | 0 | 60.949 | 0 | 0 | 0 |
| TESTI4012451 | 0 | 0 | 0 | 0 | 0 | 0 | 62.915 |
| TESTI4012646 | 60.471 | 0 | 0 | 0 | 0 | 0 | 0 |
| TESTI4013441 | 0 | 0 | 0 | 0.160 | 1.168 | 0.566 | 0.174 |
| TESTI4013474 | 0 | 0 | 0 | 0 | 0 | 0 | 2.292 |
| TESTI4013817 | 27.163 | 0 | 0 | 27.712 | 0 | 0 | 0 |
| TESTI4013894 | 0 | 0 | 0 | 14.289 | 0 | 0 | 0 |
| TESTI4014159 | 0 | 0 | 0 | 23.916 | 0 | 0 | 25.996 |
| TESTI4014694 | 2.229 | 0 | 0 | 0 | 0 | 0 | 0 |
| TESTI4014818 | 0 | 0 | 0 | 60.949 | 0 | 0 | 0 |
| TESTI4014908 | 36.051 | 0 | 0 | 0 | 0 | 0 | 0 |
| TESTI4018938 | 0 | 0 | 0 | 35.331 | 0 | 0 | 0 |
| TESTI4022158 | 53.386 | 0 | 0 | 13.617 | 0 | 0 | 14.801 |
| TESTI4024420 | 37.398 | 0 | 0 | 38.155 | 0 | 0 | 0 |
| TESTI4025268 | 60.471 | 0 | 0 | 0 | 0 | 0 | 0 |
| TESTI4025547 | 0 | 0 | 0 | 60.949 | 0 | 0 | 0 |
| TESTI4026207 | 0 | 0 | 0 | 60.949 | 0 | 0 | 0 |
| TESTI4027821 | 60.471 | 0 | 0 | 0 | 0 | 0 | 0 |
| TESTI4032090 | 60.471 | 0 | 0 | 0 | 0 | 0 | 0 |
| TESTI4032913 | 0 | 0 | 0 | 0 | 0 | 0 | 8.989 |
| TESTI4037156 | 0 | 0 | 0.980 | 0.282 | 2.060 | 0.998 | 0.307 |
| TESTI4038284 | 7.367 | 0 | 0 | 5.637 | 0 | 0 | 0 |
| TESTI4043140 | 0 | 0 | 0 | 2.829 | 10.320 | 0 | 0 |
| TESTI4044704 | 10.194 | 0 | 0 | 0 | 0 | 0 | 0 |
| TESTI4046073 | 0 | 0 | 0 | 0 | 0 | 68.459 | 0 |
| TESTI4046502 | 43.339 | 0 | 0 | 0 | 0 | 0 | 0 |
| THYMU1000002 | 0.592 | 0 | 0 | 1.812 | 0 | 0 | 0 |
| THYMU1000016 | 1.159 | 0 | 4.107 | 0.394 | 0 | 0 | 0 |
| THYMU1000029 | 0 | 0 | 0 | 55.026 | 0 | 0 | 0 |
| THYMU1000041 | 9.511 | 0 | 0 | 2.426 | 0 | 0 | 7.911 |
| THYMU1000083 | 0 | 0 | 0 | 6.119 | 0 | 0 | 0 |
| THYMU1000103 | 10.578 | 20.224 | 6.246 | 12.591 | 0 | 6.357 | 0 |
| THYMU1000105 | 0 | 0 | 0 | 15.308 | 0 | 0 | 16.639 |
| THYMU1000109 | 0 | 0 | 0 | 0 | 0 | 5.676 | 0 |
| THYMU1000144 | 9.053 | 2.967 | 0 | 1.319 | 0 | 0 | 1.434 |
| THYMU1000329 | 0 | 0 | 0 | 0 | 19.836 | 0 | 0 |
| THYMU1000359 | 0 | 0 | 0 | 0 | 0 | 1.933 | 0 |
| THYMU1000374 | 1.498 | 6.872 | 0 | 0 | 0 | 5.401 | 1.661 |
| THYMU1000490 | 5.734 | 0 | 0 | 5.850 | 0 | 0 | 0 |
| THYMU2000057 | 0 | 0 | 0 | 0 | 0 | 0 | 1.504 |
| THYMU2000138 | 0 | 0 | 0 | 0 | 0 | 0 | 46.995 |
| THYMU2000155 | 0 | 0 | 0 | 0 | 0 | 0 | 57.080 |
| THYMU2000236 | 0 | 0 | 0 | 0 | 81.697 | 0 | 0 |
| THYMU2000382 | 0 | 1.391 | 0 | 0.618 | 0 | 0 | 2.016 |
| THYMU2000436 | 0 | 4.228 | 0 | 0 | 3.429 | 6.645 | 0 |
| THYMU2000684 | 0 | 0 | 0 | 4.248 | 0 | 0 | 9.234 |
| THYMU2000775 | 0 | 0 | 0 | 0 | 0 | 33.759 | 0 |
| THYMU2000946 | 4.340 | 0 | 5.125 | 4.428 | 5.385 | 0 | 3.209 |
| THYMU2000971 | 0 | 0 | 0 | 0 | 0 | 0 | 57.080 |
| THYMU2001007 | 0 | 6.224 | 0 | 0 | 0 | 9.782 | 9.025 |
| THYMU2001018 | 9.032 | 0 | 0 | 0 | 0 | 0 | 0 |
| THYMU2001090 | 21.252 | 0 | 0 | 0 | 0 | 0 | 47.135 |
| THYMU2001422 | 0 | 28.575 | 0 | 0 | 0 | 0 | 0 |
| THYMU2001515 | 0 | 0 | 0 | 14.878 | 0 | 0 | 0 |
| THYMU2001868 | 0 | 73.343 | 0 | 0 | 0 | 0 | 0 |
| THYMU2002080 | 0 | 0 | 0 | 0 | 0 | 0 | 33.007 |
| THYMU2002583 | 3.628 | 0 | 0 | 0 | 1.929 | 0 | 2.299 |
| THYMU2002815 | 33.041 | 0 | 0 | 0 | 0 | 0 | 0 |
| THYMU2003012 | 2.629 | 4.021 | 3.105 | 6.259 | 9.785 | 0 | 3.887 |
| THYMU2003046 | 0 | 0 | 0 | 1.961 | 0 | 0 | 2.132 |
| THYMU2003133 | 0 | 0 | 0 | 0 | 0 | 0 | 3.848 |
| THYMU2003419 | 1.507 | 1.152 | 0 | 0 | 0 | 0 | 0 |
| THYMU2003446 | 14.310 | 5.472 | 0 | 9.733 | 0 | 0 | 18.514 |
| THYMU2003542 | 0 | 0 | 7.656 | 0 | 8.043 | 0 | 2.397 |
| THYMU2003891 | 0 | 0.335 | 0.517 | 0 | 0 | 0.526 | 0.324 |
| THYMU2003907 | 0 | 0 | 0 | 0 | 0 | 0 | 12.576 |
| THYMU2003932 | 0 | 0 | 0 | 0 | 0 | 0 | 21.450 |
| THYMU2003981 | 0.522 | 0 | 1.850 | 2.664 | 0 | 0 | 0.579 |
| THYMU2004152 | 0.993 | 0 | 3.517 | 0 | 0 | 0 | 1.101 |
| THYMU2004344 | 3.832 | 0 | 4.526 | 5.213 | 4.755 | 0 | 7.083 |
| THYMU2004410 | 3.832 | 4.396 | 0 | 0 | 0 | 0 | 4.250 |
| THYMU2004452 | 23.112 | 0 | 0 | 0 | 0 | 0 | 0 |
| THYMU2004635 | 0 | 0 | 0 | 55.026 | 0 | 0 | 0 |
| THYMU2004733 | 0 | 0 | 0 | 0 | 0 | 0 | 6.211 |
| THYMU2004906 | 0 | 0 | 44.401 | 0 | 0 | 0 | 0 |
| THYMU2005546 | 0 | 0 | 0 | 0.558 | 0 | 3.944 | 0.607 |
| THYMU2005997 | 16.102 | 0 | 57.044 | 0 | 0 | 0 | 0 |
| THYMU2006505 | 5.460 | 3.132 | 0 | 0 | 5.081 | 4.922 | 0 |
| THYMU2006610 | 0 | 0 | 0 | 0 | 0 | 0 | 30.115 |
| THYMU2006813 | 3.165 | 0 | 0 | 1.614 | 5.890 | 0 | 7.019 |
| THYMU2007036 | 0 | 0 | 0 | 0 | 0 | 0 | 8.562 |
| THYMU2007112 | 4.573 | 0 | 0 | 0 | 0 | 0 | 0 |
| THYMU2007467 | 4.039 | 0 | 0 | 0 | 0 | 0 | 0 |
| THYMU2007729 | 0 | 0 | 0 | 0 | 18.191 | 17.622 | 5.420 |
| THYMU2007886 | 0 | 3.177 | 4.905 | 0 | 0 | 0 | 3.071 |
| THYMU2008035 | 54.530 | 0 | 0 | 0 | 0 | 0 | 0 |
| THYMU2008049 | 0 | 0 | 0 | 0 | 0 | 0 | 25.048 |
| THYMU2008149 | 0 | 0 | 0 | 0 | 0 | 0 | 6.532 |
| THYMU2008321 | 2.891 | 13.264 | 10.241 | 2.949 | 0 | 0 | 9.617 |
| THYMU2008686 | 0 | 0 | 0 | 0 | 0 | 43.941 | 0 |
| THYMU2009338 | 0 | 0 | 0 | 0 | 0 | 0 | 57.080 |
| THYMU2009658 | 0.701 | 1.607 | 0 | 0 | 1.304 | 1.263 | 0 |
| THYMU2010041 | 0 | 0 | 0 | 19.023 | 0 | 0 | 0 |
| THYMU2010192 | 0 | 0 | 0 | 0 | 0 | 0 | 57.080 |
| THYMU2010831 | 54.530 | 0 | 0 | 0 | 0 | 0 | 0 |
| THYMU2011482 | 3.070 | 0 | 0 | 0 | 0 | 11.068 | 6.808 |
| THYMU2012024 | 2.980 | 0 | 0 | 0 | 0 | 0 | 0 |
| THYMU2012104 | 27.053 | 0 | 0 | 0 | 0 | 0 | 0 |
| THYMU2012625 | 0 | 2.383 | 0 | 0 | 3.866 | 0 | 0 |
| THYMU2012826 | 22.147 | 0 | 0 | 22.595 | 0 | 0 | 0 |
| THYMU2013051 | 0 | 0 | 0 | 0 | 0 | 81.218 | 0 |
| THYMU2013136 | 0.818 | 0 | 0 | 0 | 0 | 0 | 0 |
| THYMU2013916 | 1.109 | 2.545 | 3.929 | 3.395 | 0 | 3.999 | 3.690 |
| THYMU2014167 | 0 | 0 | 0 | 0 | 0 | 19.814 | 6.094 |
| THYMU2014183 | 0 | 0 | 0 | 0 | 0 | 0 | 10.774 |
| THYMU2015762 | 0 | 2.274 | 0 | 1.011 | 0 | 3.575 | 0 |
| THYMU2015918 | 0 | 0 | 0 | 55.026 | 0 | 0 | 0 |
| THYMU2016968 | 0 | 0 | 0 | 7.541 | 0 | 0 | 0 |
| THYMU2019021 | 14.579 | 0 | 17.216 | 4.958 | 0 | 0 | 0 |
| THYMU2020883 | 0 | 0 | 0 | 0 | 0 | 0 | 57.080 |
| THYMU2020959 | 11.085 | 25.432 | 0 | 0 | 0 | 0 | 12.293 |
| THYMU2023576 | 15.940 | 0 | 0 | 0 | 0 | 57.476 | 0 |
| THYMU2023943 | 54.530 | 0 | 0 | 0 | 0 | 0 | 0 |
| THYMU2023967 | 0 | 0 | 0 | 31.165 | 0 | 0 | 0 |
| THYMU2025319 | 33.041 | 0 | 0 | 0 | 0 | 0 | 0 |
| THYMU2025325 | 0 | 29.879 | 46.138 | 0 | 0 | 0 | 0 |
| THYMU2025707 | 0 | 0 | 0 | 9.440 | 0 | 0 | 20.521 |
| THYMU2026835 | 0 | 0 | 0 | 55.026 | 0 | 0 | 0 |
| THYMU2027125 | 0.179 | 0.411 | 0 | 0 | 0 | 4.518 | 0.198 |
| THYMU2028629 | 0 | 0 | 0 | 0 | 0 | 0 | 20.303 |
| THYMU2031341 | 0 | 0 | 0 | 7.040 | 0 | 0 | 0 |
| THYMU2031890 | 0 | 0 | 0 | 0 | 0 | 64.019 | 0 |
| THYMU2032696 | 0 | 2.411 | 0 | 0 | 0 | 3.789 | 0 |
| THYMU2032825 | 0 | 0 | 0 | 0 | 0 | 0 | 20.620 |
| THYMU2033263 | 0 | 0 | 0 | 0 | 0 | 0 | 12.337 |
| THYMU2033308 | 13.964 | 0 | 0 | 0 | 0 | 0 | 0 |
| THYMU2033787 | 0 | 0 | 0 | 0 | 0 | 0 | 57.080 |
| THYMU2035735 | 1.319 | 3.027 | 0 | 2.692 | 0 | 0 | 0 |
| THYMU2036215 | 0 | 0 | 0 | 23.470 | 0 | 0 | 0 |
| THYMU2036461 | 0 | 0 | 0 | 15.474 | 0 | 0 | 0 |
| THYMU2037226 | 0 | 0 | 0 | 34.964 | 0 | 0 | 0 |
| THYMU2038220 | 40.401 | 0 | 0 | 0 | 0 | 0 | 22.401 |
| THYMU2039315 | 54.530 | 0 | 0 | 0 | 0 | 0 | 0 |
| THYMU2039634 | 54.530 | 0 | 0 | 0 | 0 | 0 | 0 |
| THYMU2040114 | 0 | 73.343 | 0 | 0 | 0 | 0 | 0 |
| THYMU3000360 | 39.314 | 0 | 0 | 0 | 0 | 0 | 0 |
| THYMU3000420 | 39.314 | 0 | 0 | 0 | 0 | 0 | 0 |
| THYMU3000776 | 15.604 | 0 | 0 | 0 | 0 | 0 | 0 |
| THYMU3001379 | 0 | 0 | 0 | 25.695 | 0 | 0 | 27.929 |
| THYMU3003007 | 0 | 0 | 0 | 19.391 | 0 | 0 | 0 |
| THYMU3003350 | 54.530 | 0 | 0 | 0 | 0 | 0 | 0 |
| THYMU3003965 | 0 | 0 | 0 | 55.026 | 0 | 0 | 0 |
| THYMU3004835 | 0 | 0 | 0 | 20.041 | 0 | 0 | 21.784 |
| THYMU3005427 | 0 | 54.147 | 0 | 0 | 0 | 0 | 26.173 |
| THYMU3005491 | 0 | 0 | 0 | 0 | 0 | 0 | 57.080 |
| THYMU3005629 | 0 | 0 | 0 | 0 | 0 | 0 | 16.756 |
| THYMU3007137 | 0 | 0 | 0 | 37.956 | 0 | 0 | 0 |
| THYMU3007308 | 0 | 0 | 0 | 39.793 | 0 | 0 | 0 |
| THYMU3007423 | 29.497 | 0 | 0 | 10.031 | 0 | 0 | 10.904 |
| THYMU3008105 | 0 | 0 | 0 | 20.662 | 0 | 0 | 0 |
| THYMU3008171 | 20.170 | 0 | 0 | 20.579 | 0 | 0 | 0 |
| THYMU3009006 | 0 | 0 | 0 | 0 | 0 | 0 | 20.574 |
| THYMU3011827 | 0 | 0 | 0 | 0 | 0 | 0 | 25.758 |
| THYMU3013386 | 0.749 | 3.435 | 0 | 0.764 | 2.786 | 2.699 | 1.660 |
| THYMU3015759 | 1.126 | 0 | 0 | 1.149 | 0 | 0 | 0 |
| THYMU3021586 | 20.651 | 0 | 14.632 | 16.855 | 0 | 0 | 4.580 |
| THYMU3026000 | 49.066 | 0 | 0 | 0 | 0 | 0 | 0 |
| THYMU3026350 | 14.291 | 0 | 0 | 14.581 | 0 | 0 | 15.849 |
| THYMU3032798 | 9.668 | 0 | 0 | 0 | 0 | 0 | 4.289 |
| THYMU3032867 | 12.871 | 0 | 0 | 0 | 0 | 0 | 0 |
| THYMU3034671 | 0 | 0 | 0 | 4.866 | 0 | 0 | 0 |
| THYMU3037827 | 54.530 | 0 | 0 | 0 | 0 | 0 | 0 |
| THYMU3038214 | 39.314 | 0 | 0 | 0 | 0 | 0 | 0 |
| THYMU3041428 | 0 | 0 | 0.903 | 0 | 0 | 0 | 0 |
| THYMU3046350 | 0 | 0 | 24.471 | 0 | 0 | 0 | 15.320 |
| TKIDN1000001 | 0 | 0 | 0 | 0 | 0 | 0 | 12.342 |
| TKIDN1000044 | 0 | 0 | 0 | 9.554 | 0 | 33.765 | 10.385 |
| TKIDN1000164 | 0 | 0 | 0 | 6.308 | 0 | 0 | 13.713 |
| TKIDN1000192 | 5.525 | 0 | 19.571 | 0 | 0 | 0 | 6.127 |
| TKIDN2000246 | 3.042 | 11.166 | 16.165 | 5.897 | 10.190 | 6.581 | 7.759 |
| TKIDN2000319 | 0 | 0 | 0 | 21.607 | 0 | 0 | 0 |
| TKIDN2000521 | 29.753 | 0 | 0 | 15.178 | 0 | 0 | 0 |
| TKIDN2001662 | 10.906 | 0 | 0 | 11.127 | 0 | 0 | 0 |
| TKIDN2002318 | 5.363 | 0 | 0 | 0 | 0 | 19.339 | 0 |
| TKIDN2002738 | 0.419 | 0 | 0 | 0 | 0 | 1.512 | 0 |
| TKIDN2003059 | 0 | 0 | 0 | 21.607 | 0 | 0 | 0 |
| TKIDN2003078 | 0 | 0 | 0 | 0 | 0 | 0 | 15.358 |
| TKIDN2003597 | 0 | 0 | 0 | 39.852 | 0 | 0 | 17.327 |
| TKIDN2007605 | 0 | 0 | 0 | 12.745 | 0 | 0 | 0 |
| TKIDN2007828 | 0 | 0 | 23.636 | 0 | 0 | 0 | 0 |
| TKIDN2009330 | 0 | 0 | 0 | 0 | 0 | 0 | 23.052 |
| TKIDN2009641 | 5.782 | 0 | 0 | 0 | 21.519 | 0 | 12.823 |
| TKIDN2009794 | 14.428 | 0 | 0 | 0 | 0 | 0 | 16.001 |
| TKIDN2009889 | 35.077 | 0 | 0 | 0 | 0 | 0 | 0 |
| TKIDN2010812 | 0 | 0 | 0 | 0 | 0 | 0 | 47.291 |
| TKIDN2014570 | 21.269 | 0 | 0 | 0 | 0 | 0 | 0 |
| TKIDN2014964 | 0 | 0 | 0 | 0 | 0 | 39.516 | 0 |
| TKIDN2015071 | 38.914 | 0 | 0 | 0 | 0 | 0 | 0 |
| TKIDN2015423 | 18.462 | 1.461 | 0 | 9.742 | 2.369 | 0 | 9.884 |
| TKIDN2015788 | 5.261 | 0 | 0 | 2.684 | 0 | 0 | 2.917 |
| TLIVE2001327 | 0 | 0 | 0 | 0 | 0 | 0 | 5.201 |
| TLIVE2002046 | 5.445 | 4.164 | 0 | 1.852 | 0 | 0 | 12.076 |
| TLIVE2003200 | 0 | 0 | 0 | 12.922 | 0 | 0 | 0 |
| TOVAR2000904 | 0 | 0 | 0 | 4.474 | 0 | 0 | 0 |
| TRACH1000011 | 0 | 0 | 26.076 | 7.510 | 0 | 26.541 | 0 |
| TRACH1000018 | 0 | 0 | 0 | 0 | 76.867 | 0 | 0 |
| TRACH1000030 | 2.759 | 0 | 0 | 2.815 | 5.135 | 0 | 11.476 |
| TRACH1000181 | 4.738 | 0 | 0 | 0 | 0 | 0 | 10.507 |
| TRACH1000193 | 21.722 | 0 | 0 | 0 | 0 | 0 | 0 |
| TRACH1000205 | 19.677 | 0 | 0 | 4.015 | 0 | 0 | 0 |
| TRACH2000045 | 8.249 | 0 | 19.483 | 5.611 | 0 | 0 | 18.297 |
| TRACH2000047 | 0 | 0 | 0 | 0 | 0 | 0 | 11.879 |
| TRACH2000079 | 1.625 | 1.243 | 1.919 | 0.553 | 2.016 | 0 | 1.802 |
| TRACH2000237 | 0 | 0 | 0 | 5.485 | 0 | 0 | 5.962 |
| TRACH2000248 | 2.791 | 1.601 | 0 | 2.135 | 0 | 0 | 0.774 |
| TRACH2000321 | 0.983 | 0 | 0 | 1.003 | 0 | 0 | 0 |
| TRACH2000359 | 0 | 0 | 0 | 3.042 | 0 | 0 | 0 |
| TRACH2000393 | 0 | 0 | 0 | 0 | 0 | 0 | 37.734 |
| TRACH2000420 | 0 | 0 | 0 | 3.421 | 0 | 0 | 0 |
| TRACH2000461 | 12.002 | 0 | 6.074 | 8.746 | 0 | 0 | 3.803 |
| TRACH2000472 | 1.708 | 0 | 0 | 0.871 | 0 | 0 | 0.947 |
| TRACH2000502 | 47.167 | 0 | 0 | 0 | 0 | 0 | 0 |
| TRACH2000540 | 0 | 0 | 0 | 0 | 0 | 0 | 3.160 |
| TRACH2000780 | 6.118 | 0 | 3.096 | 0.892 | 0 | 0 | 0.969 |
| TRACH2000920 | 0 | 0 | 0 | 0 | 12.339 | 11.954 | 3.676 |
| TRACH2000926 | 0 | 0 | 0 | 2.847 | 10.384 | 0 | 0 |
| TRACH2000959 | 0 | 0.560 | 0 | 0 | 0 | 0 | 0.271 |
| TRACH2000972 | 0 | 2.719 | 2.099 | 0 | 0 | 0 | 1.314 |
| TRACH2001021 | 0 | 0 | 1.483 | 0 | 0 | 0 | 1.393 |
| TRACH2001085 | 0 | 0 | 0 | 0 | 76.867 | 0 | 0 |
| TRACH2001109 | 29.509 | 3.361 | 2.224 | 22.633 | 2.337 | 0 | 5.802 |
| TRACH2001289 | 1.360 | 0.780 | 0 | 0.694 | 2.530 | 1.226 | 0.377 |
| TRACH2001314 | 16.450 | 0 | 0 | 14.918 | 0 | 0 | 4.054 |
| TRACH2001339 | 0 | 67.193 | 0 | 0 | 0 | 0 | 0 |
| TRACH2001341 | 6.374 | 0 | 0 | 6.503 | 0 | 0 | 0 |
| TRACH2001428 | 2.524 | 0 | 0 | 0 | 0 | 0 | 0 |
| TRACH2001432 | 1.112 | 2.552 | 0 | 0 | 0 | 4.011 | 3.701 |
| TRACH2001463 | 3.062 | 0 | 0 | 3.124 | 0 | 0 | 0 |
| TRACH2001478 | 2.902 | 0 | 2.056 | 1.184 | 0 | 0 | 0.644 |
| TRACH2001549 | 8.457 | 0 | 0 | 21.569 | 0 | 0 | 4.689 |
| TRACH2001607 | 2.009 | 6.913 | 0 | 2.049 | 3.738 | 3.621 | 1.114 |
| TRACH2001612 | 0 | 0 | 0 | 4.075 | 0 | 0 | 4.430 |
| TRACH2001646 | 0 | 51.883 | 0 | 0 | 0 | 0 | 0 |
| TRACH2001810 | 1.553 | 0 | 2.751 | 2.377 | 2.890 | 5.600 | 2.584 |
| TRACH2001898 | 0 | 2.339 | 0 | 0 | 0 | 0 | 1.131 |
| TRACH2001996 | 0 | 67.193 | 0 | 0 | 0 | 0 | 0 |
| TRACH2002100 | 0 | 0 | 0 | 0 | 0 | 0 | 38.037 |
| TRACH2002333 | 0 | 0 | 0 | 0 | 0 | 0 | 32.604 |
| TRACH2002537 | 0.927 | 2.128 | 0 | 0.946 | 3.452 | 0 | 6.171 |
| TRACH2002664 | 0 | 0 | 0 | 0 | 0 | 0 | 1.688 |
| TRACH2002803 | 0 | 0 | 0 | 0 | 23.268 | 7.514 | 0 |
| TRACH2003272 | 3.705 | 8.500 | 13.125 | 3.780 | 0 | 0 | 4.109 |
| TRACH2003323 | 7.119 | 0 | 0 | 9.079 | 0 | 0 | 3.947 |
| TRACH2003347 | 0 | 0 | 0 | 0 | 0 | 5.728 | 1.762 |
| TRACH2005541 | 0 | 0 | 0 | 9.921 | 0 | 0 | 10.783 |
| TRACH2005796 | 0 | 0 | 0 | 0 | 0 | 0 | 49.749 |
| TRACH2005811 | 2.109 | 2.419 | 0 | 2.152 | 0 | 0 | 3.508 |
| TRACH2006049 | 47.167 | 0 | 0 | 0 | 0 | 0 | 0 |
| TRACH2006378 | 8.938 | 0 | 0 | 0 | 0 | 0 | 0 |
| TRACH2007399 | 0 | 0 | 0 | 0.481 | 0 | 1.698 | 1.045 |
| TRACH2007969 | 2.538 | 0 | 0 | 0 | 0 | 0 | 0 |
| TRACH2008113 | 0 | 0 | 65.939 | 0 | 0 | 0 | 0 |
| TRACH2008300 | 10.186 | 3.895 | 6.014 | 13.857 | 0 | 12.243 | 11.296 |
| TRACH2008472 | 0 | 0 | 5.558 | 0 | 0 | 0 | 8.700 |
| TRACH2008478 | 17.859 | 0 | 0 | 0 | 0 | 0 | 0 |
| TRACH2008716 | 0 | 11.638 | 0 | 0 | 0 | 0 | 0 |
| TRACH2008891 | 21.694 | 0 | 0 | 0 | 0 | 0 | 0 |
| TRACH2009006 | 0 | 0 | 0 | 0 | 0 | 0 | 3.892 |
| TRACH2011113 | 0 | 0 | 0 | 4.817 | 0 | 0 | 5.236 |
| TRACH2011500 | 13.502 | 0 | 0 | 0 | 0 | 0 | 0 |
| TRACH2011894 | 0 | 0 | 0 | 0 | 0 | 32.063 | 0 |
| TRACH2012298 | 0 | 3.256 | 0 | 0 | 0 | 0 | 0 |
| TRACH2012376 | 0 | 6.157 | 0 | 0 | 0 | 9.677 | 2.976 |
| TRACH2012497 | 6.473 | 0 | 0 | 0 | 0 | 0 | 0 |
| TRACH2012811 | 0 | 17.886 | 0 | 0 | 0 | 0 | 34.582 |
| TRACH2013552 | 10.389 | 0 | 0 | 31.797 | 0 | 0 | 0 |
| TRACH2013585 | 23.471 | 0 | 0 | 23.946 | 0 | 0 | 0 |
| TRACH2014077 | 3.402 | 0 | 0 | 6.941 | 0 | 0 | 7.545 |
| TRACH2014124 | 2.064 | 0 | 3.657 | 0 | 0 | 0 | 3.434 |
| TRACH2014997 | 14.151 | 0 | 0 | 7.219 | 0 | 0 | 0 |
| TRACH2015823 | 2.188 | 0 | 0 | 4.464 | 0 | 0 | 2.426 |
| TRACH2015939 | 0 | 12.887 | 0 | 5.731 | 0 | 0 | 6.229 |
| TRACH2016131 | 0 | 67.193 | 0 | 0 | 0 | 0 | 0 |
| TRACH2018034 | 0 | 0 | 0 | 0 | 0 | 0 | 49.749 |
| TRACH2018317 | 2.930 | 0 | 0 | 0 | 0 | 0 | 0 |
| TRACH2018446 | 0 | 0 | 0 | 0 | 0 | 0 | 9.275 |
| TRACH2018950 | 0 | 17.231 | 0 | 0 | 0 | 0 | 0 |
| TRACH2023299 | 0 | 0 | 0 | 0 | 0 | 0 | 14.157 |
| TRACH2025535 | 5.806 | 1.332 | 0 | 2.369 | 0 | 0 | 1.288 |
| TRACH3000014 | 0 | 0 | 0 | 18.547 | 0 | 0 | 0 |
| TRACH3000134 | 36.165 | 0 | 0 | 0 | 0 | 0 | 0 |
| TRACH3000780 | 0 | 0 | 0 | 47.666 | 0 | 0 | 0 |
| TRACH3001550 | 0 | 0 | 0 | 47.666 | 0 | 0 | 0 |
| TRACH3002192 | 4.989 | 2.861 | 0 | 6.999 | 2.321 | 0 | 2.766 |
| TRACH3002605 | 0 | 0 | 0 | 47.666 | 0 | 0 | 0 |
| TRACH3002752 | 10.215 | 0 | 0 | 10.422 | 19.010 | 0 | 0 |
| TRACH3003037 | 64.100 | 0 | 0 | 0 | 0 | 0 | 0 |
| TRACH3003547 | 4.229 | 0 | 0 | 0 | 0 | 0 | 0 |
| TRACH3003872 | 2.745 | 0 | 0 | 5.600 | 10.216 | 0 | 0 |
| TRACH3004068 | 0 | 0 | 0 | 0 | 0 | 5.139 | 1.581 |
| TRACH3004424 | 17.006 | 0 | 0 | 0 | 0 | 0 | 18.859 |
| TRACH3004721 | 8.721 | 6.669 | 0 | 2.966 | 0 | 0 | 3.224 |
| TRACH3005294 | 7.428 | 0 | 0 | 0 | 0 | 0 | 0 |
| TRACH3006717 | 18.347 | 0 | 0 | 11.231 | 0 | 0 | 0 |
| TRACH3007479 | 1.075 | 0 | 0 | 0 | 0 | 0 | 0 |
| TRACH3007625 | 1.175 | 5.394 | 8.329 | 10.794 | 0 | 0 | 3.911 |
| TRACH3007689 | 47.167 | 0 | 0 | 0 | 0 | 0 | 0 |
| TRACH3008093 | 2.449 | 5.619 | 0 | 0 | 0 | 0 | 2.716 |
| TRACH3008201 | 47.167 | 0 | 0 | 0 | 0 | 0 | 0 |
| TRACH3008508 | 0 | 0 | 0 | 0 | 0 | 0 | 21.245 |
| TRACH3008632 | 25.351 | 0 | 0 | 8.621 | 0 | 0 | 9.371 |
| TRACH3009001 | 0 | 0 | 0 | 23.904 | 0 | 0 | 0 |
| TRACH3009008 | 24.495 | 0 | 0 | 0 | 0 | 0 | 0 |
| TRACH3009148 | 17.939 | 0 | 0 | 0 | 0 | 0 | 0 |
| TRACH3009455 | 47.167 | 0 | 0 | 0 | 0 | 0 | 0 |
| TRACH3010079 | 48.305 | 0 | 0 | 24.641 | 0 | 0 | 0 |
| TRACH3016805 | 4.502 | 0 | 0 | 0 | 0 | 0 | 0 |
| TRACH3022198 | 0 | 0 | 0 | 8.480 | 30.935 | 0 | 0 |
| TRACH3024342 | 47.167 | 0 | 0 | 0 | 0 | 0 | 0 |
| TRACH3024671 | 47.167 | 0 | 0 | 0 | 0 | 0 | 0 |
| TRACH3025316 | 0 | 0 | 0 | 31.291 | 0 | 0 | 0 |
| TRACH3026303 | 5.605 | 0 | 0 | 11.436 | 0 | 0 | 0 |
| TRACH3026676 | 11.631 | 26.685 | 0 | 0 | 0 | 0 | 12.899 |
| TRACH3028855 | 22.309 | 0 | 0 | 0 | 0 | 0 | 0 |
| TRACH3032570 | 47.167 | 0 | 0 | 0 | 0 | 0 | 0 |
| TRACH3036932 | 14.659 | 0 | 0 | 14.956 | 0 | 0 | 16.256 |
| TRACH3036942 | 0.244 | 0 | 0.864 | 0 | 0 | 0 | 0 |
| TRACH3037067 | 0 | 3.688 | 0 | 0 | 0 | 0 | 0 |
| TRACH3038399 | 12.435 | 0 | 0 | 0 | 0 | 0 | 13.790 |
| TSTOM1000135 | 0 | 0 | 0 | 0 | 0 | 0 | 9.361 |
| TSTOM2000235 | 0 | 0 | 0 | 4.535 | 0 | 0 | 0 |
| TUTER1000122 | 0 | 0 | 0 | 3.330 | 0 | 0 | 0 |
| TUTER1000137 | 4.294 | 0 | 0 | 0 | 0 | 0 | 0 |
| TUTER2000904 | 0 | 0 | 0 | 5.697 | 0 | 0 | 3.096 |
| UMVEN1000122 | 0 | 0 | 0 | 0.997 | 0 | 0 | 0 |
| UMVEN1000143 | 0 | 0 | 2.909 | 0 | 0 | 0 | 0 |
| UMVEN1000186 | 0 | 0.606 | 0 | 0 | 0 | 0 | 0 |
| UMVEN2000121 | 0 | 0 | 0 | 0 | 0 | 0 | 1.149 |
| UMVEN2000133 | 1.758 | 0 | 0 | 0.598 | 0 | 0 | 0.650 |
| UMVEN2000152 | 0 | 1.807 | 0 | 0 | 0 | 0 | 0.873 |
| UMVEN2000354 | 0 | 1.890 | 2.918 | 0 | 0 | 2.970 | 0.913 |
| UMVEN2000453 | 0.522 | 2.396 | 0 | 0.533 | 0 | 1.883 | 0 |
| UTERU1000096 | 0 | 0 | 0 | 0 | 75.732 | 0 | 0 |
| UTERU1000106 | 0 | 13.221 | 0 | 0 | 0 | 0 | 0 |
| UTERU1000384 | 7.127 | 0 | 0 | 0 | 0 | 0 | 7.904 |
| UTERU2000023 | 0 | 0 | 0 | 10.444 | 0 | 0 | 0 |
| UTERU2000095 | 2.069 | 0 | 1.466 | 0 | 1.540 | 0 | 1.836 |
| UTERU2000260 | 0 | 0 | 0 | 0 | 0 | 0 | 23.088 |
| UTERU2000329 | 0 | 0 | 0 | 0 | 0 | 0 | 31.736 |
| UTERU2000332 | 0 | 0 | 0 | 0 | 0 | 0 | 5.244 |
| UTERU2000418 | 0 | 65.795 | 0 | 0 | 0 | 0 | 0 |
| UTERU2000424 | 1.699 | 3.897 | 3.009 | 0 | 0 | 3.062 | 0 |
| UTERU2000541 | 31.534 | 0 | 0 | 6.434 | 0 | 0 | 6.994 |
| UTERU2000925 | 0 | 0 | 0 | 10.625 | 38.760 | 0 | 0 |
| UTERU2001110 | 0 | 0 | 0 | 0 | 75.732 | 0 | 0 |
| UTERU2001607 | 1.475 | 3.384 | 0 | 3.010 | 0 | 0 | 3.272 |
| UTERU2001658 | 0 | 0 | 0 | 9.037 | 0 | 0 | 0 |
| UTERU2002294 | 1.815 | 0 | 0 | 0 | 0 | 0 | 4.026 |
| UTERU2002410 | 0 | 2.694 | 2.080 | 0 | 0 | 2.117 | 1.302 |
| UTERU2002662 | 1.779 | 0 | 0 | 0 | 0 | 0 | 3.946 |
| UTERU2002964 | 0 | 0 | 74.813 | 0 | 0 | 0 | 0 |
| UTERU2003057 | 0 | 0 | 0 | 0 | 0 | 9.326 | 0 |
| UTERU2004037 | 0 | 0 | 0 | 0 | 0 | 0 | 2.431 |
| UTERU2004664 | 1.697 | 0 | 0 | 0 | 0 | 0 | 1.882 |
| UTERU2004807 | 0 | 2.305 | 0 | 1.025 | 0 | 0 | 0 |
| UTERU2004929 | 0 | 0 | 0 | 11.843 | 0 | 0 | 0 |
| UTERU2005074 | 0 | 0 | 0 | 0 | 0 | 0 | 12.593 |
| UTERU2005354 | 4.375 | 0 | 7.749 | 0 | 0 | 0 | 4.852 |
| UTERU2005593 | 4.490 | 0 | 0 | 0 | 4.178 | 4.047 | 4.979 |
| UTERU2005601 | 0 | 5.848 | 0 | 2.601 | 0 | 0 | 0 |
| UTERU2005621 | 0 | 0 | 15.971 | 0 | 0 | 8.128 | 0 |
| UTERU2005664 | 0 | 0 | 0 | 11.401 | 0 | 0 | 0 |
| UTERU2006115 | 7.837 | 0 | 0 | 0 | 0 | 0 | 17.383 |
| UTERU2006486 | 0 | 0 | 0 | 0 | 0 | 0 | 48.181 |
| UTERU2006524 | 0 | 0 | 0 | 46.104 | 0 | 0 | 0 |
| UTERU2006547 | 0 | 0 | 0 | 0 | 75.732 | 0 | 0 |
| UTERU2006593 | 2.953 | 0 | 0 | 1.506 | 0 | 0 | 3.275 |
| UTERU2006643 | 0 | 0 | 30.082 | 0 | 0 | 0 | 0 |
| UTERU2007004 | 0 | 0 | 0 | 0 | 0 | 75.144 | 0 |
| UTERU2007253 | 0 | 0 | 0 | 0 | 0 | 0 | 11.427 |
| UTERU2007924 | 0 | 0 | 0 | 0 | 9.955 | 9.644 | 0 |
| UTERU2008018 | 0 | 0 | 0 | 0 | 0 | 75.144 | 0 |
| UTERU2008040 | 0 | 0 | 0 | 24.014 | 0 | 0 | 0 |
| UTERU2008085 | 0.271 | 1.243 | 0.960 | 0.276 | 1.345 | 2.605 | 0.300 |
| UTERU2008130 | 0 | 0 | 0 | 46.104 | 0 | 0 | 0 |
| UTERU2008302 | 2.672 | 3.065 | 4.733 | 1.363 | 4.972 | 4.817 | 1.482 |
| UTERU2008426 | 0 | 0 | 0 | 0 | 0 | 0 | 24.416 |
| UTERU2008747 | 0 | 0 | 0 | 0 | 32.895 | 0 | 19.602 |
| UTERU2008938 | 4.922 | 0 | 22.598 | 9.160 | 0 | 5.458 | |
| UTERU2008939 | 14.854 | 0 | 0 | 15.154 | 0 | 0 | 0 |
| UTERU2009335 | 0 | 0 | 0 | 3.094 | 0 | 21.872 | 0 |
| UTERU2009435 | 26.737 | 0 | 0 | 16.367 | 0 | 0 | 5.930 |
| UTERU2009972 | 3.543 | 0 | 0 | 3.614 | 0 | 0 | 0 |
| UTERU2010320 | 0 | 0 | 74.813 | 0 | 0 | 0 | 0 |
| UTERU2010417 | 0.315 | 0.723 | 0 | 0 | 0 | 1.137 | 0.699 |
| UTERU2011287 | 0 | 0 | 0 | 33.486 | 0 | 0 | 0 |
| UTERU2011621 | 9.930 | 0 | 0 | 10.131 | 0 | 11.011 | |
| UTERU2011806 | 0 | 0 | 0 | 0 | 0 | 50.193 | 0 |
| UTERU2011906 | 13.639 | 0 | 0 | 0 | 0 | 0 | 0 |
| UTERU2012031 | 0 | 0 | 0 | 46.104 | 0 | 0 | 0 |
| UTERU2012230 | 8.799 | 4.037 | 0 | 16.159 | 0 | 0 | 13.661 |
| UTERU2012286 | 0 | 0 | 0 | 0 | 0 | 0 | 2.810 |
| UTERU2012715 | 0 | 65.795 | 0 | 0 | 0 | 0 | 0 |
| UTERU2013078 | 0 | 0 | 0 | 0 | 0 | 49.453 | 0 |
| UTERU2013586 | 0 | 0 | 23.341 | 13.444 | 0 | 0 | 0 |
| UTERU2014024 | 0 | 0 | 0 | 13.222 | 0 | 0 | 0 |
| UTERU2014167 | 0 | 0 | 0 | 0 | 0 | 0 | 1.011 |
| UTERU2014464 | 0 | 0 | 0 | 0 | 50.246 | 0 | 0 |
| UTERU2014601 | 0 | 15.889 | 0 | 0 | 0 | 0 | 0 |
| UTERU2014898 | 0 | 0 | 0 | 1.552 | 0 | 0 | 0 |
| UTERU2015108 | 0 | 0 | 0 | 6.438 | 0 | 0 | 0 |
| UTERU2015653 | 0 | 0 | 0 | 0 | 0 | 0 | 0.647 |
| UTERU2016426 | 12.709 | 0 | 0 | 0 | 0 | 0 | 14.093 |
| UTERU2016757 | 45.606 | 0 | 0 | 0 | 0 | 0 | 0 |
| UTERU2016799 | 0 | 0 | 0 | 2.967 | 0 | 0 | 6.449 |
| UTERU2016822 | 10.050 | 0 | 0 | 6.152 | 0 | 0 | 0 |
| UTERU2018200 | 0 | 0 | 0 | 0 | 0 | 0 | 53.828 |
| UTERU2018364 | 0 | 0 | 0 | 46.104 | 0 | 0 | 0 |
| UTERU2019005 | 0 | 0 | 0 | 0 | 0 | 0 | 7.162 |
| UTERU2019096 | 13.821 | 0 | 8.160 | 2.350 | 0 | 0 | 5.109 |
| UTERU2019706 | 45.606 | 0 | 0 | 0 | 0 | 0 | 0 |
| UTERU2019940 | 0 | 0 | 0 | 0 | 0 | 0 | 48.181 |
| UTERU2021163 | 0 | 0 | 0 | 46.104 | 0 | 0 | 0 |
| UTERU2021820 | 24.929 | 0 | 0 | 0 | 0 | 0 | 27.645 |
| UTERU2023039 | 45.606 | 0 | 0 | 0 | 0 | 0 | 0 |
| UTERU2023175 | 0 | 0 | 0 | 0 | 0 | 5.368 | 1.651 |
| UTERU2026203 | 45.606 | 0 | 0 | 0 | 0 | 0 | 0 |
| UTERU2028734 | 21.953 | 0 | 0 | 0 | 0 | 0 | 0 |
| UTERU2030213 | 0 | 0 | 0 | 25.133 | 0 | 0 | 0 |
| UTERU2030280 | 0 | 0 | 0 | 0 | 0 | 0 | 48.181 |
| UTERU3000298 | 4.627 | 0 | 0 | 18.881 | 0 | 0 | 5.131 |
| UTERU3000899 | 0 | 0 | 0 | 0 | 0 | 0 | 31.292 |
| UTERU3001158 | 0 | 0 | 0 | 0 | 0 | 0 | 36.825 |
| UTERU3001571 | 0 | 0 | 0 | 0 | 0 | 0 | 48.181 |
| UTERU3003135 | 0 | 0 | 0 | 33.486 | 0 | 0 | 0 |
| UTERU3003660 | 7.363 | 0 | 0 | 7.512 | 0 | 0 | 16.330 |
| UTERU3004384 | 0 | 0 | 0 | 46.104 | 0 | 0 | 0 |
| UTERU3004418 | 29.112 | 0 | 0 | 0 | 0 | 0 | 0 |
| UTERU3004709 | 0 | 0 | 0 | 0 | 0 | 0 | 48.181 |
| UTERU3005159 | 0 | 0 | 0 | 35.590 | 0 | 0 | 0 |
| UTERU3005230 | 24.419 | 0 | 0 | 0 | 0 | 0 | 0 |
| UTERU3005422 | 0 | 0 | 0 | 21.641 | 0 | 0 | 0 |
| UTERU3005767 | 1.190 | 0 | 0 | 0 | 4.430 | 0 | 2.640 |
| UTERU3005907 | 0 | 0 | 0 | 0 | 0 | 0 | 14.514 |
| UTERU3007640 | 45.606 | 0 | 0 | 0 | 0 | 0 | 0 |
| UTERU3008280 | 31.124 | 0 | 0 | 31.754 | 0 | 0 | 0 |
| UTERU3009259 | 0 | 0 | 0 | 46.104 | 0 | 0 | 0 |
| UTERU3009871 | 36.230 | 0 | 0 | 18.482 | 0 | 0 | 0 |
| UTERU3010409 | 3.461 | 0 | 0 | 7.062 | 0 | 0 | 0 |
| UTERU3012414 | 0 | 0 | 0 | 0 | 0 | 0 | 48.181 |
| UTERU3013167 | 45.606 | 0 | 0 | 0 | 0 | 0 | 0 |
| UTERU3016273 | 0 | 0 | 0 | 46.104 | 0 | 0 | 0 |
| UTERU3017995 | 0 | 0 | 0 | 0 | 0 | 0 | 12.128 |
| UTERU3018172 | 0 | 0 | 0 | 0 | 0 | 0 | 35.368 |

Genes whose expression levels were negligibly low in each of the above-described libraries are shown with their clone names below.

3NB691000018, 3NB691000085, 3NB691000113, 3NB691000129, 3NB692000012, 3NB692000029, 3NB692000102, 3NB692000154, 3NB692000276, 3NB692000305, 3NB692000429, 3NB692000484, 3NB692000545, 3NB692000973, 3NB692001002, 3NB692001034, 3NB692001040, 3NB692001123, 3NB692001267, 3NB692001339, 3NB692001349, 3NB692001366, 3NB692001408, 3NB692001459, 3NB692001471, 3NB692001496, 3NB692001501, 3NB692001507, 3NB692001511, 3NB692001519, 3NB692001520, 3NB692001538, 3NB692001548, 3NB692001637, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064, ADIPS2000069, ADIPS2000088, ADIPS2000245, ADRGL1000038, ADRGL1000067, ADRGL1000111, ADRGL1000147, ADRGL1000160, ADRGL1000165, ADRGL1000182, ADRGL2000056, ADRGL2000064, ADRGL2000074, ADRGL2000085, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002029, ADRGL2002260, ADRGL2002392, ADRGL2002753, ADRGL2002857, ADRGL2003017, ADRGL2003329, ADRGL2003552, ADRGL2003585, ADRGL2003638, ADRGL2003684, ADRGL2003773, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007974, ADRGL2008023, ADRGL2008331, ADRGL2008337, ADRGL2008535, ADRGL2008966, ADRGL2009146, ADRGL2009324, ADRGL2009489, ADRGL2009533, ADRGL2009691, ADRGL2009755, ADRGL2010315, ADRGL2010594, ADRGL2010860, ADRGL2010974, ADRGL2011190, ADRGL2011330, ADRGL2012038, ADRGL2012179, AHMSC1000138, ASTRO1000018, ASTRO1000029, ASTRO1000096, ASTRO1000165, ASTRO2000011, ASTRO2000095, ASTRO2000141, ASTRO2000191, ASTRO2000278, ASTRO2000403, ASTRO2000417, ASTRO2000533, ASTRO2000653, ASTRO2000725, ASTRO2000801, ASTRO2001001, ASTRO2001008, ASTRO2001088, ASTRO2001122, ASTRO2001227, ASTRO2001249, ASTRO2001480, ASTRO2001806, ASTRO2002024, ASTRO2002064, ASTRO2002459, ASTRO2002571, ASTRO2002632, ASTRO2002659, ASTRO2002693, ASTRO2002720, ASTRO2002743, ASTRO2002842, ASTRO2003212, ASTRO2003581, ASTRO2003632, ASTRO2003925, ASTRO2003960, ASTRO2004032, ASTRO2004051, ASTRO2004177, ASTRO2004217, ASTRO2004297, ASTRO2004584, ASTRO2004751, ASTRO2004877, ASTRO2005008, ASTRO2005081, ASTRO2005096, ASTRO2005242, ASTRO2005273, ASTRO2005343, ASTRO2005413, ASTRO2005553, ASTRO2005557, ASTRO2005575, ASTRO2005593, ASTRO2005687, ASTRO2005726, ASTRO2005896, ASTRO2005902, ASTRO2006356, ASTRO2006732, ASTRO2006747, ASTRO2006920, ASTRO2006952, ASTRO2007047, ASTRO2007221, ASTRO2007377, ASTRO2007515, ASTRO2007578, ASTRO2007643, ASTRO2007666, ASTRO2007956, ASTRO2007962, ASTRO2008239, ASTRO2008409, ASTRO2008425, ASTRO2008508, ASTRO2008777, ASTRO2008895, ASTRO2008960, ASTRO2009118, ASTRO2009177, ASTRO2009333, ASTRO2009458, ASTRO2009526, ASTRO2009879, ASTRO2010582, ASTRO2010615, ASTRO2010962, ASTRO2010996, ASTRO2011149, ASTRO2011426, ASTRO2011437, ASTRO2011461, ASTRO2011834, ASTRO2011893, ASTRO2013124, ASTRO2013671, ASTRO2013802, ASTRO2014211, ASTRO2014363, ASTRO2014498, ASTRO2014561, ASTRO2014923, ASTRO2014961, ASTRO2015162, ASTRO2015185, ASTRO2015529, ASTRO2015987, ASTRO2016044, ASTRO2016148, ASTRO2016681, ASTRO2016819, ASTRO2016847, ASTRO2016892, ASTRO2017348, ASTRO2017627, ASTRO2018373, ASTRO2019039, ASTRO2019171, ASTRO3000154, ASTRO3000172, ASTRO3000177, ASTRO3000301, ASTRO3000474, BEAST2000454, BGGI11000193, BGGI11000285, BGGI12000022, BGGI12000067, BGGI12000161, BGGI12000533, BGGI12000544, BGGI12000684, BGGI12000693, BGGI12001097, BGGI12001241, BGGI12001247, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001458, BLADE2001575, BLADE2001753, BLADE2001935, BLADE2001940, BLADE2001987, BLADE2002073, BLADE2002310, BLADE2002730, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2005036, BLADE2005038, BLADE2005229, BLADE2005459, BLADE2005792, BLADE2006043, BLADE2006415, BLADE2006607, BLADE2006826, BLADE2006849, BLADE2006947, BLADE2007033, BLADE2007666, BLADE2007722, BLADE2007735, BLADE2007744, BLADE2007799, BLADE2007923, BLADE2007958, BLADE2008281, BLADE2008454, BLADE2008461, BLADE2008479, BLADE2008539, BLADE2008809, BLADE2008995, BLADE2009452, BLADE2009479, BLADE2009593, BNGH41000104, BNGH41000118, BNGH41000153, BNGH41000169, BNGH41000177, BNGH41000216, BNGH41000235, BNGH42000130, BNGH42000132, BNGH42000405, BNGH42000675, BNGH42000791, BNGH42000815, BNGH42000994, BNGH42001406, BNGH42001576, BNGH42001724, BNGH42002244, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42006226, BNGH42006234, BNGH42007037, BNGH42007366, BNGH42007594, BNGH42007798, BNGH42008199, BNGH42008510, BNGH42008649, BNGH42008832, BNGH42008850, BNGH42008943, BNGH42009021, BRACE1000047, BRACE1000084, BRACE1000087, BRACE1000092, BRACE1000093, BRACE1000114, BRACE1000124, BRACE1000132, BRACE1000166, BRACE1000239, BRACE1000451, BRACE1000475, BRACE1000572, BRACE2000061, BRACE2000077, BRACE2000078, BRACE2000100, BRACE2000148, BRACE2000183, BRACE2000229, BRACE2000232, BRACE2000256, BRACE2000266, BRACE2000267, BRACE2000351, BRACE2000368, BRACE2000392, BRACE2000441, BRACE2000487, BRACE2000489, BRACE2000505, BRACE2000526, BRACE2000531, BRACE2000545, BRACE2000553, BRACE2000577, BRACE2000640, BRACE2000650, BRACE2000677, BRACE2000718, BRACE2000743, BRACE2000753, BRACE2000815, BRACE2000885, BRACE2000905, BRACE2000936, BRACE2000947, BRACE2000965, BRACE2000969, BRACE2000988, BRACE2001017, BRACE2001094, BRACE2001107, BRACE2001117, BRACE2001151, BRACE2001191, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001492, BRACE2001508, BRACE2001543, BRACE2001564, BRACE2001588, BRACE2001607, BRACE2001626, BRACE2001677, BRACE2001705, BRACE2001733, BRACE2001737, BRACE2001742, BRACE2001777, BRACE2001779, BRACE2001855, BRACE2001859, BRACE2001865, BRACE2001872, BRACE2001881, BRACE2001898, BRACE2001923, BRACE2001942, BRACE2001954, BRACE2001972, BRACE2001973, BRACE2002026, BRACE2002034, BRACE2002081, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002394, BRACE2002409, BRACE2002431, BRACE2002441, BRACE2002444, BRACE2002458, BRACE2002467, BRACE2002468, BRACE2002589, BRACE2002590, BRACE2002613, BRACE2002614, BRACE2002617, BRACE2002623, BRACE2002635, BRACE2002649, BRACE2002661, BRACE2002676, BRACE2002685, BRACE2002695, BRACE2002707, BRACE2002736, BRACE2002752, BRACE2002755, BRACE2002762, BRACE2002772, BRACE2002792, BRACE2002796, BRACE2002807, BRACE2002834, BRACE2002849, BRACE2002857, BRACE2002861, BRACE2002884, BRACE2002948, BRACE2003110, BRACE2003168, BRACE2003199, BRACE2003210, BRACE2003269, BRACE2003285, BRACE2003304, BRACE2003319, BRACE2003398, BRACE2003420, BRACE2003428, BRACE2003431, BRACE2003512, BRACE2003516, BRACE2003540, BRACE2003594, BRACE2003614, BRACE2003628, BRACE2003639, BRACE2003713, BRACE2003766, BRACE2003802, BRACE2003825, BRACE2003847, BRACE2003848, BRACE2003873, BRACE2003885, BRACE2003887, BRACE2003928, BRACE2003954, BRACE2003987, BRACE2005034, BRACE2005047, BRACE2005063, BRACE2005090, BRACE2005118, BRACE2005124, BRACE2005138, BRACE2005160, BRACE2005169, BRACE2005189, BRACE2005196, BRACE2005198, BRACE2005216, BRACE2005243, BRACE2005298, BRACE2005328, BRACE2005329, BRACE2005337, BRACE2005348, BRACE2005363, BRACE2005433, BRACE2005450, BRACE2005453, BRACE2005460, BRACE2005489, BRACE2005518, BRACE2005556, BRACE2005562, BRACE2005624, BRACE2005661, BRACE2005667, BRACE2005731, BRACE2005742, BRACE2005762, BRACE2005773, BRACE2005787, BRACE2005795, BRACE2005800, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006072, BRACE2006102, BRACE2006105, BRACE2006122, BRACE2006137, BRACE2006143, BRACE2006162, BRACE2006174, BRACE2006240, BRACE2006245, BRACE2006249, BRACE2006274, BRACE2006298, BRACE2006319, BRACE2006378, BRACE2006393, BRACE2006413, BRACE2006417, BRACE2006453, BRACE2006459, BRACE2006488, BRACE2006534, BRACE2006540, BRACE2006547, BRACE2006564, BRACE2006587, BRACE2006598, BRACE2006685, BRACE2006703, BRACE2006783, BRACE2006833, BRACE2006900, BRACE2006909, BRACE2006911, BRACE2006924, BRACE2006935, BRACE2006944, BRACE2006951, BRACE2006986, BRACE2007068, BRACE2007087, BRACE2007138, BRACE2007174, BRACE2007197, BRACE2007203, BRACE2007232, BRACE2007262, BRACE2007274, BRACE2007281, BRACE2007295, BRACE2007358, BRACE2007396, BRACE2007398, BRACE2007411, BRACE2007447, BRACE2007477, BRACE2007502, BRACE2007503, BRACE2007527, BRACE2007538, BRACE2007563, BRACE2007621, BRACE2007640, BRACE2007646, BRACE2007682, BRACE2007685, BRACE2007727, BRACE2007749, BRACE2007760, BRACE2007761, BRACE2007764, BRACE2007769, BRACE2007784, BRACE2007786, BRACE2007799, BRACE2007801, BRACE2007836, BRACE2007868, BRACE2007888, BRACE2007902, BRACE2007920, BRACE2007944, BRACE2008008, BRACE2008015, BRACE2008021, BRACE2008102, BRACE2008114, BRACE2008144, BRACE2008172, BRACE2008218, BRACE2008276, BRACE2008295, BRACE2008308, BRACE2008336, BRACE2008361, BRACE2008401, BRACE2008410, BRACE2008420, BRACE2008443, BRACE2008473, BRACE2008480, BRACE2008481, BRACE2008488, BRACE2008495, BRACE2008500, BRACE2008594, BRACE2008604, BRACE2008615, BRACE2008622, BRACE2008629, BRACE2008655, BRACE2008656, BRACE2008710, BRACE2008754, BRACE2008797, BRACE2008847, BRACE2008861, BRACE2008867, BRACE2008881, BRACE2008883, BRACE2008917, BRACE2008919, BRACE2008948, BRACE2008960, BRACE2008999, BRACE2009014, BRACE2009016, BRACE2009031, BRACE2009044, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009293, BRACE2009311, BRACE2009318, BRACE2009323, BRACE2.009421, BRACE2009428, BRACE2009434, BRACE2009437, BRACE2009533, BRACE2009558, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010535, BRACE2010550, BRACE2010598, BRACE2010632, BRACE2010642, BRACE2010684, BRACE2010813, BRACE2010828, BRACE2010837, BRACE2010888, BRACE2010937, BRACE2010983, BRACE2011007, BRACE2011025, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012510, BRACE2012625, BRACE2012653, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2014108, BRACE2014161, BRACE2014232, BRACE2014268, BRACE2014475, BRACE2014780, BRACE2014818, BRACE2014821, BRACE2014824, BRACE2014854, BRACE2015031, BRACE2015262, BRACE2015270, BRACE2015314, BRACE2015352, BRACE2015436, BRACE2015741, BRACE2015756, BRACE2015969, BRACE2015995, BRACE2016058, BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017410, BRACE2017438, BRACE2017580, BRACE2017587, BRACE2017720, BRACE2017872, BRACE2017929, BRACE2017985, BRACE2017992, BRACE2018443, BRACE2018448, BRACE2018568, BRACE2018700, BRACE2018727, BRACE2018759, BRACE2018782, BRACE2019044, BRACE2019244, BRACE2019258, BRACE2019371, BRACE2019467, BRACE2019510, BRACE2019528, BRACE2019618, BRACE2019696, BRACE2019703, BRACE2019843, BRACE2019865, BRACE2019872, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021721, BRACE2021936, BRACE2022030, BRACE2022158, BRACE2022196, BRACE2022270, BRACE2022328, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023268, BRACE2023284, BRACE2023404, BRACE2023451, BRACE2023540, BRACE2023633, BRACE2023727, BRACE2023744, BRACE2023800, BRACE2023801, BRACE2024074, BRACE2024222, BRACE2024610, BRACE2024627, BRACE2024826, BRACE2025018, BRACE2025316, BRACE2025333, BRACE2025452, BRACE2025492, BRACE2025710, BRACE2025796, BRACE2025992, BRACE2026054, BRACE2026131, BRACE2026141, BRACE2026293, BRACE2026294, BRACE2026404, BRACE2026675, BRACE2026725, BRACE2026836, BRACE2026920, BRACE2026971, BRACE2027221, BRACE2027258, BRACE2027312, BRACE2027382, BRACE2027389, BRACE2027408, BRACE2027770, BRACE2027879, BRACE2027970, BRACE2028171, BRACE2028410, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030345, BRACE2030736, BRACE2030883, BRACE2030884, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035441, BRACE2035485, BRACE2036005, BRACE2036096, BRACE2036743, BRACE2036830, BRACE2036834, BRACE2036900, BRACE2037294, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044263, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003413, BRACE3003595, BRACE3003698, BRACE3003866, BRACE3003920, BRACE3004058, BRACE3004113, BRACE3004150, BRACE3004205, BRACE3004358, BRACE3004435, BRACE3004477, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005870, BRACE3005903, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007322, BRACE3007472, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008720, BRACE3008772, BRACE3009044, BRACE3009075, BRACE3009090, BRACE3009127, BRACE3009197, BRACE3009265, BRACE3009297, BRACE3009377, BRACE3009392, BRACE3009539, BRACE3009574, BRACE3009701, BRACE3009708, BRACE3009993, BRACE3010076, BRACE3010079, BRACE3010086, BRACE3010221, BRACE3010283, BRACE3010315, BRACE3010416, BRACE3010435, BRACE3010702, BRACE3010862, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3019055, BRACE3019084, BRACE3019570, BRACE3019611, BRACE3019817; BRACE3019828, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3026008, BRACE3026075, BRACE3026161, BRACE3026290, BRACE3026345, BRACE3026456, BRACE3026844, BRACE3026947, BRACE3026993, BRACE3027242, BRACE3027256, BRACE3027326, BRACE3027480, BRACE3027976, BRACE3028214, BRACE3028241, BRACE3028360, BRACE3028895, BRACE3028998, BRACE3029005, BRACE3029205, BRACE3029447, BRACE3030057, BRACE3030103, BRACE3030538, BRACE3030748, BRACE3031161, BRACE3031184, BRACE3031315, BRACE3031372, BRACE3031579, BRACE3031728, BRACE3031843, BRACE3032455, BRACE3032537, BRACE3032538, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039378, BRACE3039454, BRACE3040012, BRACE3040239, BRACE3040504, BRACE3040644, BRACE3040856, BRACE3041059, BRACE3041162, BRACE3041827, BRACE3042046, BRACE3042210, BRACE3042409, BRACE3042594, BRACE3043597, BRACE3044090, BRACE3044172, BRACE3044247, BRACE3044377, BRACE3044389, BRACE3044495, BRACE3045011, BRACE3045033, BRACE3045145, BRACE3045424, BRACE3045708, BRACE3046049, BRACE3046152, BRACE3046294, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2001038, BRALZ2001279, BRALZ2001350, BRALZ2001445, BRALZ2001559, BRALZ2001743, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008763, BRALZ2008869, BRALZ2008930, BRALZ2010186, BRALZ2010842, BRALZ2011337, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRAMY1000095, BRAMY1000098, BRAMY1000130, BRAMY1000157, BRAMY2000021, BRAMY2000076, BRAMY2000086, BRAMY2000151, BRAMY2000181, BRAMY2000196, BRAMY2000231, BRAMY2000277, BRAMY2000653, BRAMY2000814, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002493, BRAMY2002525, BRAMY2002584, BRAMY2002604, BRAMY2002638, BRAMY2002739, BRAMY2002853, BRAMY2002862, BRAMY2002956, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004058, BRAMY2004183, BRAMY2004335, BRAMY2004351, BRAMY2004352, BRAMY2004492, BRAMY2004537, BRAMY2004542, BRAMY2004756, BRAMY2004771, BRAMY2005064, BRAMY2005094, BRAMY2005151, BRAMY2005182, BRAMY2005244, BRAMY2005391, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006411, BRAMY2006459, BRAMY2006466, BRAMY2006659, BRAMY2006779, BRAMY2006809, BRAMY2006879, BRAMY2007043, BRAMY2007110, BRAMY2007185, BRAMY2007249, BRAMY2007308, BRAMY2007411, BRAMY2007458, BRAMY2007486, BRAMY2007546, BRAMY2007610, BRAMY2007653, BRAMY2007753, BRAMY2007859, BRAMY2008333, BRAMY2008382, BRAMY2008583, BRAMY2008979, BRAMY2009034, BRAMY2009105, BRAMY2009123, BRAMY2009192, BRAMY2009344, BRAMY2009349, BRAMY2009394, BRAMY2009528, BRAMY2009693, BRAMY2010068, BRAMY2010135, BRAMY2010145, BRAMY2010171, BRAMY2010208, BRAMY2010239, BRAMY2010290, BRAMY2010366, BRAMY2010395, BRAMY2010441, BRAMY2010581, BRAMY2010798, BRAMY2010808, BRAMY2010919, BRAMY2011064, BRAMY2011178, BRAMY2011196, BRAMY2011216, BRAMY2011253, BRAMY2011480, BRAMY2011639, BRAMY2011679, BRAMY2011811, BRAMY2011846, BRAMY2011872, BRAMY2011961, BRAMY2012017, BRAMY2012091, BRAMY2012119, BRAMY2012162, BRAMY2012277, BRAMY2012426, BRAMY2012497, BRAMY2012517, BRAMY2012555, BRAMY2012568, BRAMY2012581, BRAMY2012691, BRAMY2012721, BRAMY2012731, BRAMY2012776, BRAMY2012880, BRAMY2013200, BRAMY2013216, BRAMY2013405, BRAMY2013414, BRAMY2013572, BRAMY2013590, BRAMY2013621, BRAMY2013659, BRAMY2013722, BRAMY2013756, BRAMY2013944, BRAMY2013975, BRAMY2014019, BRAMY2014387, BRAMY2014469, BRAMY2014541, BRAMY2014813, BRAMY2015211, BRAMY2015311, BRAMY2015503, BRAMY2015516, BRAMY2015551, BRAMY2015559, BRAMY2015688, BRAMY2015705, BRAMY2015782, BRAMY2015855, BRAMY2015890, BRAMY2015925, BRAMY2016002, BRAMY2016020, BRAMY2016070, BRAMY2016327, BRAMY2016386, BRAMY2016706, BRAMY2016771, BRAMY2016938, BRAMY2016953, BRAMY2017014, BRAMY2017249, BRAMY2017348, BRAMY2017450, BRAMY2017455, BRAMY2017528, BRAMY2018027, BRAMY2018106, BRAMY2018122, BRAMY2018126, BRAMY2018272, BRAMY2018273, BRAMY2018279, BRAMY2018308, BRAMY2018396, BRAMY2018467, BRAMY2019055, BRAMY2019277, BRAMY2019509, BRAMY2019643, BRAMY2019985, BRAMY2020050, BRAMY2020058, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021310, BRAMY2021388, BRAMY2021498, BRAMY2021523, BRAMY2021710, BRAMY2021732, BRAMY2021746, BRAMY2021962, BRAMY2021982, BRAMY2022052, BRAMY2022263, BRAMY2022301, BRAMY2022320, BRAMY2022383, BRAMY2022445, BRAMY2022454, BRAMY2022532, BRAMY2022796, BRAMY2022929, BRAMY2022984, BRAMY2023060, BRAMY2023110, BRAMY2023161, BRAMY2023172, BRAMY2023238, BRAMY2023327, BRAMY2023358, BRAMY2023482, BRAMY2023514, BRAMY2023719, BRAMY2023729, BRAMY2023786, BRAMY2023852, BRAMY2023863, BRAMY2023939, BRAMY2024073, BRAMY2024374, BRAMY2024449, BRAMY2024489, BRAMY2024497, BRAMY2024514, BRAMY2024530, BRAMY2024535, BRAMY2024545, BRAMY2024576, BRAMY2024715, BRAMY2024728, BRAMY2024849, BRAMY2025024, BRAMY2025032, BRAMY2025175, BRAMY2025218, BRAMY2025230, BRAMY2025277, BRAMY2025495, BRAMY2025812, BRAMY2025996, BRAMY2026009, BRAMY2026091, BRAMY2026300, BRAMY2026347, BRAMY2026395, BRAMY2026405, BRAMY2026538, BRAMY2026685, BRAMY2026713, BRAMY2026778, BRAMY2026824, BRAMY2026899, BRAMY2026902, BRAMY2026904, BRAMY2026976, BRAMY2027073, BRAMY2027140, BRAMY2027242, BRAMY2027396, BRAMY2027451, BRAMY2027461, BRAMY2027467, BRAMY2027714, BRAMY2027752, BRAMY2027782, BRAMY2028072, BRAMY2028188, BRAMY2028282, BRAMY2028472, BRAMY2028491, BRAMY2028565, BRAMY2028593, BRAMY2028682, BRAMY2028783, BRAMY2028856, BRAMY2029204, BRAMY2029602, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031377, BRAMY2031442, BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035801, BRAMY2035869, BRAMY2035923, BRAMY2036079, BRAMY2036254, BRAMY2036266, BRAMY2036387, BRAMY2036396, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037971, BRAMY2038100, BRAMY2038128, BRAMY2038317, BRAMY2038475, BRAMY2038484, BRAMY2038832, BRAMY2038846, BRAMY2038887, BRAMY2038904, BRAMY2039287, BRAMY2039300, BRAMY2039314, BRAMY2039341, BRAMY2039695, BRAMY2039872, BRAMY2040051, BRAMY2040159, BRAMY2040478, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046984, BRAMY2046989, BRAMY2047169, BRAMY2047280, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002508, BRAMY3002620, BRAMY3002805, BRAMY3002886, BRAMY3003026, BRAMY3003109, BRAMY3003205, BRAMY3003310, BRAMY3003566, BRAMY3003723, BRAMY3004364, BRAMY3004672, BRAMY3004900, BRAMY3004919, BRAMY3005656, BRAMY3005932, BRAMY3006032, BRAMY3006297, BRAMY3007206, BRAMY3007350, BRAMY3007449, BRAMY3007471, BRAMY3007609, BRAMY3007723, BRAMY3007968, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009904, BRAMY3010321, BRAMY3010411, BRAMY3010603, BRAMY4000277, BRAMY4000962, BRAMY4001173, BRAMY4001200, BRAMY4001234, BRAMY4001449, BRAMY4001652, BRAMY4001913, BRAMY4002575, BRAMY4002628, BRASW1000053, CD34C2000051, CD34C2000152, CD34C2000175, CD34C2000443, CD34C3000314, CD34C3000494, CERVX1000042, CERVX2000812, CERVX2000968, CERVX2002006, CERVX2002013, CERVX2002430, CERVX2002770, CHONS1000058, CHONS2000797, CHONS2001287, CHONS2001382, CHONS2001797, CHONS2001834, CHONS2002419, COLON1000030, COLON1000084, COLON1000135, COLON2000470, COLON2000557, COLON2000568, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2008105, COLON2009337, COLON2009499, CORDB1000140, CORDB2000061, CORDB2000127, CORDB2000541, CTONG1000009, CTONG1000020, CTONG1000022, CTONG1000040, CTONG1000056, CTONG1000062, CTONG1000086, CTONG1000093, CTONG1000097, CTONG1000113, CTONG1000137, CTONG1000165, CTONG1000175, CTONG1000181, CTONG1000220, CTONG1000277, CTONG1000283, CTONG1000288, CTONG1000302, CTONG1000341, CTONG1000476, CTONG1000488, CTONG1000508, CTONG1000540, CTONG2000034, CTONG2000201, CTONG2000218, CTONG2000279, CTONG2000303, CTONG2000330, CTONG2000395, CTONG2000465, CTONG2000480, CTONG2000561, CTONG2000589, CTONG2000766, CTONG2000771, CTONG2000819, CTONG2000846, CTONG2000855, CTONG2000977, CTONG2001139, CTONG2001366, CTONG2001413, CTONG2001428, CTONG2001513, CTONG2001524, CTONG2001598, CTONG2001681, CTONG2001724, CTONG2001749, CTONG2001820, CTONG2001858, CTONG2001877, CTONG2001932, CTONG2001955, CTONG2002066, CTONG2002143, CTONG2002395, CTONG2002417, CTONG2002418, CTONG2002453, CTONG2002520, CTONG2002558, CTONG2002590, CTONG2002709, CTONG2002721, CTONG2002816, CTONG2002820, CTONG2002903, CTONG2002965, CTONG2002999, CTONG2003028, CTONG2003100, CTONG2003115, CTONG2003189, CTONG2003245, CTONG2003293, CTONG2003348, CTONG2003350, CTONG2003361, CTONG2003372, CTONG2003375, CTONG2003427, CTONG2003517, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004397, CTONG2004454, CTONG2004550, CTONG2004669, CTONG2004716, CTONG2004912, CTONG2004941, CTONG2004948, CTONG2005049, CTONG2005110, CTONG2005265, CTONG2005290, CTONG2005374, CTONG2005399, CTONG2005468, CTONG2005567, CTONG2005585, CTONG2005615, CTONG2005635, CTONG2005775, CTONG2005890, CTONG2005904, CTONG2005913, CTONG2006129, CTONG2006223, CTONG2006273, CTONG2006310, CTONG2006515, CTONG2006524, CTONG2006568, CTONG2006611, CTONG2006620, CTONG2006666, CTONG2006691, CTONG2006798, CTONG2006836, CTONG2006932, CTONG2006942, CTONG2006964, CTONG2007007, CTONG2007009, CTONG2007091, CTONG2007175, CTONG2007259, CTONG2007272, CTONG2007297, CTONG2007441, CTONG2007474, CTONG2007623, CTONG2007898, CTONG2007959, CTONG2007970, CTONG2008184, CTONG2008262, CTONG2008269, CTONG2008343, CTONG2008402, CTONG2008466, CTONG2008562, CTONG2008577, CTONG2008595, CTONG2008621, CTONG2008720, CTONG2008773, CTONG2008792, CTONG2008989, CTONG2009132, CTONG2009156, CTONG2009181, CTONG2009257, CTONG2009258, CTONG2009268, CTONG2009270, CTONG2009395, CTONG2009440, CTONG2009527, CTONG2009531, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009955, CTONG2009958, CTONG2009963, CTONG2010018, CTONG2010024, CTONG2010116, CTONG2010133, CTONG2010148, CTONG2010276, CTONG2010330, CTONG2010508, CTONG2010566, CTONG2010623, CTONG2010649, CTONG2010803, CTONG2011429, CTONG2011474, CTONG2011676, CTONG2011716, CTONG2011765, CTONG2011779, CTONG2011815, CTONG2011822, CTONG2011825, CTONG2011920, CTONG2011985, CTONG2012029, CTONG2012077, CTONG2012101, CTONG2012123, CTONG2012158, CTONG2012230, CTONG2012401, CTONG2012422, CTONG2012425, CTONG2012447, CTONG2012453, CTONG2012473, CTONG2012476, CTONG2012533, CTONG2012554, CTONG2012564, CTONG2012661, CTONG2012724, CTONG2012738, CTONG2012758, CTONG2012843, CTONG2012901, CTONG2013128, CTONG2013149, CTONG2013156, CTONG2013178, CTONG2013222, CTONG2013284, CTONG2013339, CTONG2013352, CTONG2013393, CTONG2013511, CTONG2013630, CTONG2013803, CTONG2013985, CTONG2013986, CTONG2014032, CTONG2014058, CTONG2014191, CTONG2014206, CTONG2014234, CTONG2014264, CTONG2014280, CTONG2014491, CTONG2014630, CTONG2014635, CTONG2014646, CTONG2014705, CTONG2014946, CTONG2014954, CTONG2014959, CTONG2014966, CTONG2014995, CTONG2015316, CTONG2015358, CTONG2015434, CTONG2015540, CTONG2015596, CTONG2015633, CTONG2015678, CTONG2015717, CTONG2015804, CTONG2015866, CTONG2015914, CTONG2015953, CTONG2016185, CTONG2016217, CTONG2016355, CTONG2016505, CTONG2016559, CTONG2016658, CTONG2016775, CTONG2016797, CTONG2016824, CTONG2016846, CTONG2016904, CTONG2016942, CTONG2016953, CTONG2017021, CTONG2017292, CTONG2017429, CTONG2017444, CTONG2017458, CTONG2017604, CTONG2017650, CTONG2017798, CTONG2017804, CTONG2017814, CTONG2017910, CTONG2017939, CTONG2017989, CTONG2018062, CTONG2018069, CTONG2018147, CTONG2018217, CTONG2018279, CTONG2018334, CTONG2018343, CTONG2018379, CTONG2018483, CTONG2018629, CTONG2018632, CTONG2018652, CTONG2018653, CTONG2018655, CTONG2018735, CTONG2018808, CTONG2018843, CTONG2018900, CTONG2018976, CTONG2019144, CTONG2019232, CTONG2019248, CTONG2019400, CTONG2019652, CTONG2019704, CTONG2019822, CTONG2019833, CTONG2020031, CTONG2020108, CTONG2020127, CTONG2020171, CTONG2020411, CTONG2020522, CTONG2020638, CTONG2020806, CTONG2020974, CTONG2021024, CTONG2021132, CTONG2021168, CTONG2021289, CTONG2022153, CTONG2022601, CTONG2022835, CTONG2023449, CTONG2023512, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027327, CTONG2027361, CTONG2027591, CTONG2027736, CTONG2027783, CTONG2027828, CTONG2027959, CTONG2028097, CTONG2028518, CTONG2028687, CTONG2028758, CTONG3000686, CTONG3000707, CTONG3000896, CTONG3001420, CTONG3001501, CTONG3001560, CTONG3001605, CTONG3002020, CTONG3002127, CTONG3002370, CTONG3002433, CTONG3002518, CTONG3002552, CTONG3002588, CTONG3002674, CTONG3002728, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004317, CTONG3004397, CTONG3004550, CTONG3004576, CTONG3004607, CTONG3004712, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009227, CTONG3009287, CTONG3009385, D3OST1000066, D3OST1000109, D3OST1000238, D3OST2000184, D3OST2000618, D3OST2000654, D3OST2000734, D3OST2001328, D3OST2001669, D3OST2001670, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000507, D9OST2000278, D9OST2000440, D9OST2000869, D9OST2001319, D9OST2001433, D9OST2002397, D9OST2002673, D9OST2003108, D9OST2003137, D9OST2003397, D9OST2003580, D9OST2003594, D9OST2003791, D9OST2003989, D9OST2004018, D9OST2004073, D9OST2004417, DFNES1000003, DFNES1000107, DFNES1000185, DFNES2000143, DFNES2000146, DFNES2000212, DFNES2000426, DFNES2000432, DFNES2000471, DFNES2000913, DFNES2001108, DFNES2001177, DFNES2001404, DFNES2001564, DFNES2001565, DFNES2001647, DFNES2001800, DFNES2002220, DFNES2002509, DFNES2002550, DFNES2002588, DFNES2002817, DFNES2002966, DFNES2003081, DFNES2003192, DFNES2003255, DFNES2003742, DFNES2004346, DFNES2004354, DFNES2004383, DFNES2004608, DFNES2004684, DFNES2005266, DFNES2005527, DFNES2005540, DFNES2005766, DFNES2005779, DFNES2005875, DFNES2006346, DFNES2006944, DFNES2007299, DFNES2007332, DFNES2007641, DFNES2007757, DFNES2008280, DFNES2008881, DFNES2010502, DFNES2011192, DFNES2011221, DFNES2011239, ERLTF1000021, ERLTF2000324, ERLTF2000465, FCBBF1000023, FCBBF1000038, FCBBF1000053, FCBBF1000069, FCBBF1000115, FCBBF1000117, FCBBF1000125, FCBBF1000131, FCBBF1000155, FCBBF1000157, FCBBF1000171, FCBBF1000174, FCBBF1000182, FCBBF1000198, FCBBF1000206, FCBBF1000220, FCBBF1000232, FCBBF1000233, FCBBF1000243, FCBBF1000270, FCBBF1000280, FCBBF1000297, FCBBF1000305, FCBBF1000314, FCBBF1000322, FCBBF1000349, FCBBF1000374, FCBBF1000377, FCBBF1000395, FCBBF1000397, FCBBF1000425, FCBBF1000437, FCBBF1000506, FCBBF1000546, FCBBF1000550, FCBBF1000581, FCBBF1000582, FCBBF1000598, FCBBF1000604, FCBBF1000618, FCBBF1000627, FCBBF1000675, FCBBF1000686, FCBBF1000687, FCBBF1000691, FCBBF1000732, FCBBF2000094, FCBBF2000105, FCBBF2000123, FCBBF2000195, FCBBF2000214, FCBBF2000232, FCBBF2000259, FCBBF2000276, FCBBF2000291, FCBBF2000379, FCBBF2000473, FCBBF2000502, FCBBF2000576, FCBBF2000677, FCBBF2000678, FCBBF2000782, FCBBF2000808, FCBBF2000815, FCBBF2000825, FCBBF2000951, FCBBF2001088, FCBBF2001116, FCBBF2001188, FCBBF2001238, FCBBF2001243, FCBBF2001299, FCBBF2001309, FCBBF2001427, FCBBF2001529, FCBBF2001672, FCBBF2001675, FCBBF2001718, FCBBF2001720, FCBBF2001868, FCBBF2002044, FCBBF2002111, FCBBF2002281, FCBBF2002370, FCBBF2002541, FCBBF2002626, FCBBF2002897, FCBBF2002898, FCBBF2002904, FCBBF2002953, FCBBF2003083, FCBBF2003102, FCBBF2003269, FCBBF2003297, FCBBF2003395, FCBBF2003528, FCBBF2003549, FCBBF2003636, FCBBF2003823, FCBBF2003895, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005966, FCBBF2006131, FCBBF2006380, FCBBF2006418, FCBBF2006452, FCBBF2007012, FCBBF2007186, FCBBF2007265, FCBBF2007302, FCBBF2007556, FCBBF2007610, FCBBF2007633, FCBBF2007840, FCBBF3000102, FCBBF3000110, FCBBF3000120, FCBBF3000175, FCBBF3000227, FCBBF3000233, FCBBF3000269, FCBBF3000361, FCBBF3000367, FCBBF3000462, FCBBF3000473, FCBBF3000501, FCBBF3000518, FCBBF3000550, FCBBF3000847, FCBBF3001018, FCBBF3001020, FCBBF3001235, FCBBF3001281, FCBBF3001302, FCBBF3001594, FCBBF3001818, FCBBF3001855, FCBBF3001912, FCBBF3001914, FCBBF3001918, FCBBF3001924, FCBBF3002163, FCBBF3002188, FCBBF3002190, FCBBF3002268, FCBBF3002475, FCBBF3002556, FCBBF3002592, FCBBF3002658, FCBBF3002782, FCBBF3002925, FCBBF3003216, FCBBF3003305, FCBBF3003373, FCBBF3003435, FCBBF3003475, FCBBF3003481, FCBBF3003902, FCBBF3004021, FCBBF3004041, FCBBF3004261, FCBBF3004390, FCBBF3004409, FCBBF3004502, FCBBF3004842, FCBBF3004847, FCBBF3005160, FCBBF3005218, FCBBF3005444, FCBBF3005497, FCBBF3005698, FCBBF3005729, FCBBF3006171, FCBBF3006249, FCBBF3006288, FCBBF3006399, FCBBF3006628, FCBBF3007077, FCBBF3007150, FCBBF3007152, FCBBF3007242, FCBBF3007244, FCBBF3007248, FCBBF3007453, FCBBF3007540, FCBBF3007597, FCBBF3007604, FCBBF3007829, FCBBF3007855, FCBBF3008073, FCBBF3008100, FCBBF3008251, FCBBF3008311, FCBBF3008362, FCBBF3008382, FCBBF3008475, FCBBF3008556, FCBBF3008689, FCBBF3008748, FCBBF3008870, FCBBF3008938, FCBBF3008944, FCBBF3009256, FCBBF3009317, FCBBF3009428, FCBBF3009464, FCBBF3009703, FCBBF3009717, FCBBF3009888, FCBBF3009949, FCBBF3010008, FCBBF3010124, FCBBF3010130, FCBBF3010149, FCBBF3010211, FCBBF3010361, FCBBF3010508, FCBBF3010544, FCBBF3010586, FCBBF3010601, FCBBF3010665, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012443, FCBBF3012588, FCBBF3012667, FCBBF3012842, FCBBF3012901, FCBBF3013041, FCBBF3013058, FCBBF3013205, FCBBF3013307, FCBBF3013341, FCBBF3013589, FCBBF3013623, FCBBF3013846, FCBBF3014229, FCBBF3014355, FCBBF3014567, FCBBF3014770, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016644, FCBBF3016987, FCBBF3017024, FCBBF3017059, FCBBF3017071, FCBBF3017123, FCBBF3017233, FCBBF3017288, FCBBF3017535, FCBBF3017681, FCBBF3017729, FCBBF3017873, FCBBF3017974, FCBBF3018453, FCBBF3018591, FCBBF3018753, FCBBF3018826, FCBBF3019085, FCBBF3019320, FCBBF3019437, FCBBF3019455, FCBBF3019564, FCBBF3019569, FCBBF3019570, FCBBF3019663, FCBBF3019714, FCBBF3019716, FCBBF3019784, FCBBF3019867, FCBBF3019961, FCBBF3020030, FCBBF3020138, FCBBF3020140, FCBBF3020163, FCBBF3020232, FCBBF3020599, FCBBF3021191, FCBBF3021221, FCBBF3021501, FCBBF3021506, FCBBF3021524, FCBBF3021940, FCBBF3021985, FCBBF3022005, FCBBF3022291, FCBBF3022311, FCBBF3022504, FCBBF3022765, FCBBF3022894, FCBBF3023061, FCBBF3023078, FCBBF3023372, FCBBF3023443, FCBBF3023599, FCBBF3023704, FCBBF3023887, FCBBF3023895, FCBBF3024096, FCBBF3024225, FCBBF3024364, FCBBF3024793, FCBBF3024911, FCBBF3025098, FCBBF3025252, FCBBF3025280, FCBBF3025674, FCBBF3025730, FCBBF3025905, FCBBF3026021, FCBBF3026048, FCBBF3026251, FCBBF3026678, FCBBF3027104, FCBBF3027199, FCBBF3027328, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028472, FCBBF3028528, FCBBF3028582, FCBBF3028671, FCBBF3028794, FCBBF4000017, FCBBF4000061, FCBBF4000076, FCBBF4000173, FCBBF4000192, FCBBF4000193, FCBBF4000282, FCBBF4000399, FCBBF4000415, FCBBF4000500, FCBBF4000529, FCBBF4000548, FCBBF5000041, FCBBF5000353, FCBBF5000384, FCBBF5000483, FCBBF5000495, FEBRA1000005, FEBRA1000008, FEBRA1000030, FEBRA1000138, FEBRA1000148, FEBRA1000149, FEBRA1000183, FEBRA2000021, FEBRA2000035, FEBRA2000126, FEBRA2000220, FEBRA2000228, FEBRA2000282, FEBRA2000286, FEBRA2000311, FEBRA2000321, FEBRA2000377, FEBRA2000378, FEBRA2000391, FEBRA2000394, FEBRA2000397, FEBRA2000399, FEBRA2000404, FEBRA2000409, FEBRA2000415, FEBRA2000436, FEBRA2000438, FEBRA2000454, FEBRA2000468, FEBRA2000491, FEBRA2000504, FEBRA2000510, FEBRA2000532, FEBRA2000575, FEBRA2000656, FEBRA2000659, FEBRA2000690, FEBRA2000727, FEBRA2000734, FEBRA2000782, FEBRA2000790, FEBRA2000803, FEBRA2000805, FEBRA2000809, FEBRA2000856, FEBRA2000862, FEBRA2000881, FEBRA2000909, FEBRA2000917, FEBRA2000928, FEBRA2000936, FEBRA2000937, FEBRA2000959, FEBRA2000972, FEBRA2001012, FEBRA2001020, FEBRA2001093, FEBRA2001124, FEBRA2001133, FEBRA2001161, FEBRA2001175, FEBRA2001217, FEBRA2001267, FEBRA2001351, FEBRA2001383, FEBRA2001438, FEBRA2001487, FEBRA2001523, FEBRA2001561, FEBRA2001584, FEBRA2001590, FEBRA2001610, FEBRA2001620, FEBRA2001659, FEBRA2001705, FEBRA2001752, FEBRA2001772, FEBRA2001790, FEBRA2001828, FEBRA2001914, FEBRA2001989, FEBRA2002009, FEBRA2002086, FEBRA2002109, FEBRA2002191, FEBRA2002260, FEBRA2002399, FEBRA2002410, FEBRA2002429, FEBRA2002508, FEBRA2002525, FEBRA2002527, FEBRA2002552, FEBRA2002574, FEBRA2002601, FEBRA2002628, FEBRA2002662, FEBRA2002682, FEBRA2002727, FEBRA2002783, FEBRA2002818, FEBRA2002858, FEBRA2002882, FEBRA2002897, FEBRA2002908, FEBRA2002962, FEBRA2002986, FEBRA2003054, FEBRA2003155, FEBRA2003163, FEBRA2003181, FEBRA2003198, FEBRA2003253, FEBRA2003275, FEBRA2003308, FEBRA2003327, FEBRA2003429, FEBRA2003454, FEBRA2003468, FEBRA2003520, FEBRA2003524, FEBRA2003541, FEBRA2003675, FEBRA2003711, FEBRA2003822, FEBRA2003897, FEBRA2003907, FEBRA2004023, FEBRA2004026, FEBRA2004029, FEBRA2004053, FEBRA2004056, FEBRA2004110, FEBRA2004191, FEBRA2004202, FEBRA2004237, FEBRA2004268, FEBRA2004329, FEBRA2004331, FEBRA2004412, FEBRA2004443, FEBRA2004538, FEBRA2004620, FEBRA2004651, FEBRA2004767, FEBRA2004813, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005377, FEBRA2005380, FEBRA2005458, FEBRA2005701, FEBRA2005726, FEBRA2005734, FEBRA2005778, FEBRA2005787, FEBRA2005998, FEBRA2006061, FEBRA2006112, FEBRA2006150, FEBRA2006165, FEBRA2006315, FEBRA2006346, FEBRA2006354, FEBRA2006379, FEBRA2006396, FEBRA2006409, FEBRA2006418, FEBRA2006427, FEBRA2006476, FEBRA2006491, FEBRA2006664, FEBRA2006667, FEBRA2006726, FEBRA2006736, FEBRA2006793, FEBRA2006873, FEBRA2006942, FEBRA2006977, FEBRA2007017, FEBRA2007200, FEBRA2007247, FEBRA2007280, FEBRA2007414, FEBRA2007436, FEBRA2007449, FEBRA2007458, FEBRA2007566, FEBRA2007620, FEBRA2007708, FEBRA2007714, FEBRA2007793, FEBRA2007931, FEBRA2008009, FEBRA2008134, FEBRA2008135, FEBRA2008159, FEBRA2008165, FEBRA2008172, FEBRA2008265, FEBRA2008282, FEBRA2008311, FEBRA2008341, FEBRA2008459, FEBRA2008468, FEBRA2008475, FEBRA2008481, FEBRA2008542, FEBRA2008660, FEBRA2008681, FEBRA2008692, FEBRA2008741, FEBRA2008836, FEBRA2008859, FEBRA2008866, FEBRA2008881, FEBRA2008928, FEBRA2008983, FEBRA2009016, FEBRA2009029, FEBRA2009074, FEBRA2009117, FEBRA2009120, FEBRA2009162, FEBRA2009240, FEBRA2009265, FEBRA2009276, FEBRA2009327, FEBRA2009385, FEBRA2009416, FEBRA2009437, FEBRA2009478, FEBRA2009507, FEBRA2009541, FEBRA2009567, FEBRA2009731, FEBRA2009754, FEBRA2009780, FEBRA2010141, FEBRA2010299, FEBRA2010484, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013905, FEBRA2014010, FEBRA2014122, FEBRA2014198, FEBRA2014213, FEBRA2014443, FEBRA2014578, FEBRA2014939, FEBRA2015076, FEBRA2015175, FEBRA2015292, FEBRA2015588, FEBRA2016112, FEBRA2016621, FEBRA2016654, FEBRA2016739, FEBRA2017098, FEBRA2017138, FEBRA2017154, FEBRA2017200, FEBRA2017223, FEBRA2017333, FEBRA2017502, FEBRA2017736, FEBRA2017780, FEBRA2017811, FEBRA2017850, FEBRA2018051, FEBRA2018203, FEBRA2018407, FEBRA2018433, FEBRA2018476, FEBRA2018746, FEBRA2019172, FEBRA2019298, FEBRA2019389, FEBRA2019457, FEBRA2019519, FEBRA2019582, FEBRA2019637, FEBRA2019663, FEBRA2019690, FEBRA2020406, FEBRA2020566, FEBRA2020582, FEBRA2020668, FEBRA2020801, FEBRA2021032, FEBRA2021134, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022148, FEBRA2022204, FEBRA2022255, FEBRA2022322, FEBRA2022601, FEBRA2022729, FEBRA2022787, FEBRA2022911, FEBRA2022956, FEBRA2022963, FEBRA2022972, FEBRA2023285, FEBRA2023351, FEBRA2023399, FEBRA2023498, FEBRA2023550, FEBRA2023721, FEBRA2023989, FEBRA2023990, FEBRA2024150, FEBRA2024343, FEBRA2024693, FEBRA2024727, FEBRA2024744, FEBRA2024797, FEBRA2024987, FEBRA2025249, FEBRA2025463, FEBRA2025477, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEHRT2000325, FEHRT2000364, FEKID1000018, FELIV1000101, FELIV1000137, FELIV1000164, FELNG2000241, HCASM1000021, HCASM1000050, HCASM1000061, HCASM1000130, HCASM2000156, HCASM2000202, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003099, HCASM2003212, HCASM2003237, HCASM2003357, HCASM2003415, HCASM2006131, HCASM2006338, HCASM2006359, HCASM2006632, HCASM2007317, HCASM2007737, HCASM2007773, HCASM2008154, HCASM2008536, HCASM2009405, HCASM2009424, HCASM2009463, HCASM2009588, HCHON1000142, HCHON1000176, HCHON2000038, HCHON2000100, HCHON2000156, HCHON2000199, HCHON2000265, HCHON2000271, HCHON2000315, HCHON2000323, HCHON2000344, HCHON2000373, HCHON2000395, HCHON2000417, HCHON2000418, HCHON2000503, HCHON2000508, HCHON2000660, HCHON2000673, HCHON2000676, HCHON2000699, HCHON2000705, HCHON2000817, HCHON2000818, HCHON2000819, HCHON2000832, HCHON2000851, HCHON2000898, HCHON2000935, HCHON2000942, HCHON2000956, HCHON2000994, HCHON2001050, HCHON2001110, HCHON2001116, HCHON2001161, HCHON2001214, HCHON2001233, HCHON2001269, HCHON2001450, HCHON2001453, HCHON2001497, HCHON2001505, HCHON2001519, HCHON2001523, HCHON2001546, HCHON2001548, HCHON2001577, HCHON2001598, HCHON2001604, HCHON2001607, HCHON2001646, HCHON2001712, HCHON2001768, HCHON2002123, HCHON2002194, HCHON2002241, HCHON2002354, HCHON2002496, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005987, HCHON2006459, HCHON2006714, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HEART1000010, HEART1000088, HEART1000099, HEART1000102, HEART1000132, HEART1000139, HEART1000151, HEART1000173, HEART2000024, HEART2000035, HEART2000099, HEART2000298, HEART2000306, HEART2000309, HEART2000411, HEART2000418, HEART2000448, HEART2000492, HEART2000506, HEART2000520, HEART2000568, HEART2000611, HEART2001680, HEART2001756, HEART2002129, HEART2002184, HEART2002220, HEART2002432, HEART2002531, HEART2002598, HEART2002717, HEART2003027, HEART2003432, HEART2003440, HEART2003781, HEART2003836, HEART2004570, HEART2004764, HEART2004931, HEART2004940, HEART2004941, HEART2004980, HEART2005244, HEART2005310, HEART2005581, HEART2005610, HEART2006131, HEART2006310, HEART2006334, HEART2006487, HEART2006557, HEART2006787, HEART2006789, HEART2006909, HEART2007443, HEART2007767, HEART2008108, HEART2008111, HEART2008257, HEART2008994, HEART2009599, HEART2009680, HEART2010391, HEART2010492, HEART2010495, HELAC2000158, HELAC2000301, HHDPC1000001, HHDPC2000070, HHDPC2000102, HHDPC2000315, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2006460, HHDPC2006862, HHDPC2006927, HHDPC2007302, HHDPC2008123, HHDPC2008185, HHDPC2008279, HHDPC2008297, HHDPC2008414, HHDPC2008474, HHDPC2008843, HHDPC2009114, HHDPC2009178, HHDPC2009208, HHDPC2009272, HLUNG1000017, HLUNG1000024, HLUNG1000030, HLUNG1000034, HLUNG1000037, HLUNG1000053, HLUNG1000064, HLUNG1000076, HLUNG1000084, HLUNG1000091, HLUNG1000099, HLUNG1000104, HLUNG1000105, HLUNG1000110, HLUNG1000151, HLUNG1000169, HLUNG2000004, HLUNG2000014, HLUNG2000027, HLUNG2000068, HLUNG2000116, HLUNG2000125, HLUNG2000130, HLUNG2000142, HLUNG2000174, HLUNG2000176, HLUNG2000217, HLUNG2000255, HLUNG2000281, HLUNG2000340, HLUNG2000362, HLUNG2000363, HLUNG2000431, HLUNG2000480, HLUNG2000489, HLUNG2000549, HLUNG2000557, HLUNG2000570, HLUNG2000761, HLUNG2000777, HLUNG2000870, HLUNG2000926, HLUNG2000950, HLUNG2000955, HLUNG2000970, HLUNG2001013, HLUNG2001126, HLUNG2001144, HLUNG2001214, HLUNG2001507, HLUNG2001633, HLUNG2001677, HLUNG2001712, HLUNG2001814, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002562, HLUNG2002659, HLUNG2002707, HLUNG2002942, HLUNG2002949, HLUNG2002958, HLUNG2002982, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004154, HLUNG2004217, HLUNG2004273, HLUNG2004388, HLUNG2004416, HLUNG2004521, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006599, HLUNG2006614, HLUNG2006671, HLUNG2006781, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008637, HLUNG2008833, HLUNG2008875, HLUNG2008885, HLUNG2008958, HLUNG2008959, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000137, HSYRA1000148, HSYRA2000044, HSYRA2000135, HSYRA2000221, HSYRA2000237, HSYRA2000253, HSYRA2000371, HSYRA2000424, HSYRA2000507, HSYRA2000510, HSYRA2000605, HSYRA2000706, HSYRA2000760, HSYRA2000792, HSYRA2000832, HSYRA2000927, HSYRA2001003, HSYRA2001103, HSYRA2001142, HSYRA2001153, HSYRA2001225, HSYRA2001255, HSYRA2001332, HSYRA2001420, HSYRA2001552, HSYRA2001567, HSYRA2001580, HSYRA2001631, HSYRA2002103, HSYRA2002590, HSYRA2002741, HSYRA2003168, HSYRA2004550, HSYRA2004858, HSYRA2005456, HSYRA2005496, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000161, IMR321000165, IMR321000193, IMR321000207, IMR321000230, IMR322000010, IMR322000107, IMR322000164, IMR322000178, IMR322000374, IMR322000379, IMR322000383, IMR322000418, IMR322000656, IMR322000672, IMR322000742, IMR322000791, IMR322000811, IMR322000859, IMR322000863, IMR322000917, IMR322000919, IMR322000953, IMR322000973, IMR322000984, IMR322001018, IMR322001049, IMR322001167, IMR322001245, IMR322001318, IMR322001322, IMR322001438, IMR322001452, IMR322001491, IMR322001534, IMR322001665, IMR322001670, IMR322001724, IMR322002110, IMR322002470, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322015523, IMR322016146, IMR322017049, IMR322018117, IMR322018192, KIDNE1000008, KIDNE1000012, KIDNE1000015, KIDNE1000028, KIDNE1000036, KIDNE1000064, KIDNE1000121, KIDNE1000143, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000287, KIDNE2000328, KIDNE2000335, KIDNE2000349, KIDNE2000375, KIDNE2000394, KIDNE2000493, KIDNE2000510, KIDNE2000513, KIDNE2000519, KIDNE200.0555, KIDNE2000624, KIDNE2000665, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2001140, KIDNE2001269, KIDNE2001361, KIDNE2001373, KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002198, KIDNE2002262, KIDNE2002265, KIDNE2002438, KIDNE2002483, KIDNE2002768, KIDNE2002795, KIDNE2002839, KIDNE2002845, KIDNE2002872, KIDNE2002882, KIDNE2002980, KIDNE2002991, KIDNE2003185, KIDNE2003188, KIDNE2003233, KIDNE2003316, KIDNE2003335, KIDNE2003357, KIDNE2003373, KIDNE2003377, KIDNE2003507, KIDNE2003752, KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005603, KIDNE2005629, KIDNE2005781, KIDNE2005798, KIDNE2005854, KIDNE2005908, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006652, KIDNE2006687, KIDNE2006733, KIDNE2006775, KIDNE2006811, KIDNE2006820, KIDNE2006880, KIDNE2007005, KIDNE2007040, KIDNE2007077, KIDNE2007186, KIDNE2007222, KIDNE2007356, KIDNE2007422, KIDNE2007569, KIDNE2007810, KIDNE2007811, KIDNE2007910, KIDNE2007944, KIDNE2008022, KIDNE2008072, KIDNE2008116, KIDNE2008117, KIDNE2008218, KIDNE2008315, KIDNE2008378, KIDNE2008446, KIDNE2008473, KIDNE2008480, KIDNE2008649, KIDNE2008697, KIDNE2008788, KIDNE2008795, KIDNE2008824, KIDNE2008832, KIDNE2008869, KIDNE2008975, KIDNE2009019, KIDNE2009109, KIDNE2009180, KIDNE2009191, KIDNE2009332, KIDNE2009426, KIDNE2009467, KIDNE2009490, KIDNE2009553, KIDNE2009628, KIDNE2009647, KIDNE2009676, KIDNE2009723, KIDNE2010007, KIDNE2010052, KIDNE2010084, KIDNE2010137, KIDNE2010151, KIDNE2010264, KIDNE2010271, KIDNE2010419, KIDNE2010574, KIDNE2010674, KIDNE2010739, KIDNE2010762, KIDNE2010863, KIDNE2010973, KIDNE2010989, KIDNE2011200, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012453, KIDNE2012563, KIDNE2012601, KIDNE2012613, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016388, KIDNE2016464, KIDNE2016539, KIDNE2016570, KIDNE2016918, KIDNE2017007, KIDNE2017052, KIDNE2017151, KIDNE2017153, KIDNE2017315, KIDNE2017332, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018462, KIDNE2018617, KIDNE2018678, KIDNE2018727, KIDNE2018863, KIDNE2018891, KIDNE2018989, KIDNE2018996, KIDNE2019074, KIDNE2019187, LIVER1000050, LIVER1000055, LIVER1000058, LIVER1000063, LIVER1000067, LIVER1000073, LIVER1000082, LIVER1000099, LIVER1000111, LIVER1000139, LIVER1000155, LIVER1000165, LIVER1000175, LIVER1000224, LIVER1000278, LIVER1000303, LIVER1000397, LIVER1000421, LIVER1000433, LIVER1000479, LIVER1000482, LIVER1000542, LIVER2000003, LIVER2000037, LIVER2000158, LIVER2000216, LIVER2000237, LIVER2000247, LIVER2000360, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2002644, LIVER2002660, LIVER2002702, LIVER2003008, LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005198, LIVER2005218, LIVER2005376, LIVER2005385, LIVER2005512, LIVER2005520, LIVER2005527, LIVER2005544, LIVER2005625, LIVER2005728, LIVER2005981, LIVER2006006, LIVER2006251, LIVER2006469, LIVER2006644, LIVER2006764, LIVER2007415, LIVER2007548, LIVER2007568, LIVER2007721, LIVER2007783, LIVER2008053, LIVER2008473, LIVER2008580, LIVER2008706, LIVER2008945, LIVER2008998, LIVER2009110, LIVER2009118, LIVER2009554, LYMPB1000141, LYMPB1000158, LYMPB2000083, MAMGL1000096, MAMGL1000132, MAMGL1000151, MAMGL1000173, MAMGL1000178, MAMGL1000182, MAMGL1000184, MESAN1000035, MESAN1000121, MESAN1000147, MESAN1000180, MESAN2000023, MESAN2000026, MESAN2000067, MESAN2000167, MESAN2000291, MESAN2000337, MESAN2000434, MESAN2000457, MESAN2000462, MESAN2000620, MESAN2000711, MESAN2000815, MESAN2000894, MESAN2000909, MESAN2001450, MESAN2001770, MESAN2001979, MESAN2001980, MESAN2002113, MESAN2002147, MESAN2002424, MESAN2002687, MESAN2002709, MESAN2002790, MESAN2002978, MESAN2003035, MESAN2003037, MESAN2003039, MESAN2003058, MESAN2003101, MESAN2003322, MESAN2003490, MESAN2003529, MESAN2003662, MESAN2003851, MESAN2004138, MESAN2004288, MESAN2004575, MESAN2005303, MESAN2005371, MESAN2005633, MESAN2005724, MESAN2005808, MESAN2005811, MESAN2006022, MESAN2006328, MESAN2006580, MESAN2006599, MESAN2006971, MESAN2007032, MESAN2007440, MESAN2007597, MESAN2008222, MESAN2008334, MESAN2008415, MESAN2008460, MESAN2008536, MESAN2008679, MESAN2008729, MESAN2008821, MESAN2009019, MESAN2009105, MESAN2009311, MESAN2009418, MESAN2009522, MESAN2010031, MESAN2010114, MESAN2010312, MESAN2010321, MESAN2010681, MESAN2011545, MESAN2011597, MESAN2011627, MESAN2011977, MESAN2012586, MESAN2012708, MESAN2012735, MESAN2013019, MESAN2013022, MESAN2013183, MESAN2013211, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016552, MESAN2016965, MESAN2017152, MESAN2017373, MESAN2017417, MESAN2018209, MESAN2018576, MESAN2018670, MESAN2018699, MESTC1000147, MESTC2000150, MESTC2000153, MESTC2000170, N1ESE2000698, NB9N41000121, NB9N41000135, NB9N41000146, NB9N42000104, NB9N42000122, NB9N42000196, NB9N42000314, NB9N42000495, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000187, NHNPC2000212, NHNPC2000606, NHNPC2000877, NHNPC2001108, NHNPC2001223, NHNPC2001312, NHNPC2001816, NHNPC2002565, NHNPC2002749, NOVAR1000091, NOVAR1000102, NOVAR2000136, NOVAR2000352, NOVAR2000412, NOVAR2000710, NOVAR2000783, NOVAR2000962, NOVAR2001108, NOVAR2001620, NOVAR2001876, NOVAR2002039, NT2NE1000014, NT2NE1000023, NT2NE1000024, NT2NE1000059, NT2NE1000063, NT2NE1000083, NT2NE1000120, NT2NE1000122, NT2NE1000175, NT2NE1000185, NT2NE2000038, NT2NE2000054, NT2NE2000057, NT2NE2000064, NT2NE2000109, NT2NE2000137, NT2NE2000156, NT2NE2000222, NT2NE2000259, NT2NE2000284, NT2NE2000299, NT2NE2000353, NT2NE2000356, NT2NE2000369, NT2NE2000374, NT2NE2000390, NT2NE2000455, NT2NE2000470, NT2NE2000488, NT2NE2000517, NT2NE2000536, NT2NE2000546, NT2NE2000550, NT2NE2000586, NT2NE2000612, NT2NE2000658, NT2NE2000706, NT2NE2000802, NT2NE2000808, NT2NE2000864, NT2NE2000913, NT2NE2000950, NT2NE2000951, NT2NE2001005, NT2NE2001021, NT2NE2001142, NT2NE2001181, NT2NE2001188, NT2NE2001191, NT2NE2001247, NT2NE2001252, NT2NE2001324, NT2NE2001326, NT2NE2001372, NT2NE2001428, NT2NE2001432, NT2NE2001524, NT2NE2001530, NT2NE2001617, NT2NE2001623, NT2NE2001626, NT2NE2001634, NT2NE2001666, NT2NE2001677, NT2NE2001697, NT2NE2001698, NT2NE2001723, NT2NE2001725, NT2NE2001874, NT2NE2001889, NT2NE2001923, NT2NE2002100, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003252, NT2NE2003308, NT2NE2003309, NT2NE2003315, NT2NE2003393, NT2NE2003408, NT2NE2003457, NT2NE2003540, NT2NE2003571, NT2NE2003705, NT2NE2003752, NT2NE2004378, NT2NE2004490, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005441, NT2NE2005517, NT2NE2005537, NT2NE2005555, NT2NE2005676, NT2NE2005720, NT2NE2005784, NT2NE2005804, NT2NE2005821, NT2NE2005890, NT2NE2005921, NT2NE2005968, NT2NE2006087, NT2NE2006103, NT2NE2006217, NT2NE2006288, NT2NE2006382, NT2NE2006387, NT2NE2006427, NT2NE2006432, NT2NE2006458, NT2NE2006531, NT2NE2006659, NT2NE2006784, NT2NE2006813, NT2NE2006894, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009426, NT2NE2009608, NT2NE2009691, NT2NE2010105, NT2NE2010150, NT2NE2010400, NT2NE2010632, NT2NE2010633, NT2NE2010654, NT2NE2010781, NT2NE2010854, NT2NE2011033, NT2NE2011036, NT2NE2011107, NT2NE2011154, NT2NE2011411, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2013019, NT2NE2013081, NT2NE2013189, NT2NE2013217, NT2NE2013547, NT2NE2014013, NT2NE2014028, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2016041, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018739, NT2NE2018916, NT2NE2019056, NT2NE2019076, NT2NE2019099, NT2RI1000027, NT2RI1000035, NT2RI1000164, NT2RI1000172, NT2RI2000004, NT2RI2000007, NT2RI2000117, NT2RI2000128, NT2RI2000133, NT2RI2000173, NT2RI2000187, NT2RI2000270, NT2RI2000276, NT2RI2000282, NT2RI2000313, NT2RI2000341, NT2RI2000348, NT2RI2000412, NT2RI2000421, NT2RI2000480, NT2RI2000575, NT2RI2000588, NT2RI2000597, NT2RI2000669, NT2RI2000688, NT2RI2000689, NT2RI2000704, NT2RI2000738, NT2RI2000822, NT2RI2000841, NT2RI2000865, NT2RI2000932, NT2RI2000987, NT2RI2001010, NT2RI2001017, NT2RI2001030, NT2RI2001083, NT2RI2001091, NT2RI2001167, NT2RI2001178, NT2RI2001235, NT2RI2001244, NT2RI2001307, NT2RI2001342, NT2RI2001385, NT2RI2001409, NT2RI2001410, NT2RI2001425, NT2RI2001449, NT2RI2001474, NT2RI2001519, NT2RI2001595, NT2RI2001624, NT2RI2001769, NT2RI2001782, NT2RI2001836, NT2RI2001866, NT2RI2002022, NT2RI2002041, NT2RI2002117, NT2RI2002120, NT2RI2002153, NT2RI2002179, NT2RI2002252, NT2RI2002260, NT2RI2002270, NT2RI2002334, NT2RI2002447, NT2RI2002481, NT2RI2002507, NT2RI2002517, NT2RI2002554, NT2RI2002564, NT2RI2002585, NT2RI2002729, NT2RI2002802, NT2RI2002852, NT2RI2002865, NT2RI2002923, NT2RI2002970, NT2RI2003019, NT2RI2003067, NT2RI2003184, NT2RI2003360, NT2RI2003383, NT2RI2003392, NT2RI2003399, NT2RI2003407, NT2RI2003419, NT2RI2003487, NT2RI2003667, NT2RI2003678, NT2RI2003884, NT2RI2003973, NT2RI2003993, NT2RI2004059, NT2RI2004079, NT2RI2004093, NT2RI2004136, NT2RI2004157, NT2RI2004190, NT2RI2004209, NT2RI2004284, NT2RI2004290, NT2RI2004312, NT2RI2004381, NT2RI2004442, NT2RI2004468, NT2RI2004618, NT2RI2004818, NT2RI2004840, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005130, NT2RI2005166, NT2RI2005246, NT2RI2005315, NT2RI2005358, NT2RI2005473, NT2RI2005520, NT2RI2005621, NT2RI2005628, NT2RI2005647, NT2RI2005670, NT2RI2005710, NT2RI2005772, NT2RI2005966, NT2RI2006070, NT2RI2006081, NT2RI2006126, NT2RI2006127, NT2RI2006183, NT2RI2006257, NT2RI2006294, NT2RI2006345, NT2RI2006403, NT2RI2006445, NT2RI2006487, NT2RI2006506, NT2RI2006565, NT2RI2006659, NT2RI2006662, NT2RI2006667, NT2RI2006679, NT2RI2006682, NT2RI2006686, NT2RI2006716, NT2RI2006762, NT2RI2006788, NT2RI2006838, NT2RI2006853, NT2RI2006855, NT2RI2006973, NT2RI2007046, NT2RI2007048, NT2RI2007116, NT2RI2007214, NT2RI2007303, NT2RI2007334, NT2RI2007386, NT2RI2007406, NT2RI2007439, NT2RI2007469, NT2RI2007507, NT2RI2007629, NT2RI2007723, NT2RI2007729, NT2RI2007751, NT2RI2007754, NT2RI2007827, NT2RI2007853, NT2RI2007877, NT2RI2007879, NT2RI2007891, NT2RI2007956, NT2RI2007965, NT2RI2008007, NT2RI2008050, NT2RI2008188, NT2RI2008204, NT2RI2008221, NT2RI2008233, NT2RI2008455, NT2RI2008502, NT2RI2008598, NT2RI2008714, NT2RI2008801, NT2RI2008808, NT2RI2008841, NT2RI2008952, NT2RI2009005, NT2RI2009083, NT2RI2009101, NT2RI2009103, NT2RI2009144, NT2RI2009151, NT2RI2009215, NT2RI2009259, NT2RI2009289, NT2RI2009301, NT2RI2009517, NT2RI2009585, NT2RI2009595, NT2RI2009855, NT2RI2010283, NT2RI2010508, NT2RI2011422, NT2RI2011683, NT2RI2011803, NT2RI2012350, NT2RI2012990, NT2RI2013357, NT2RI2013373, NT2RI2014247, NT2RI2014551, NT2RI2014683, NT2RI2015239, NT2RI2016128, NT2RI2016134, NT2RI2017529, NT2RI2018363, NT2RI2018448, NT2RI2018883, NT2RI2018950, NT2RI2019751, NT2RI2019826, NT2RI2020390, NT2RI2020703, NT2RI2021463, NT2RI2022468, NT2RI2022584, NT2RI2023303, NT2RI2023671, NT2RI2024008, NT2RI2024460, NT2RI2024496, NT2RI2024935, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001458, NT2RI3001573, NT2RI3001967, NT2RI3002303, NT2RI3002557, NT2RI3003027, NT2RI3003031, NT2RI3003095, NT2RI3003104, NT2RI3003162, NT2RI3003362, NT2RI3003382, NT2RI3003409, NT2RI3004133, NT2RI3004381, NT2RI3005202, NT2RI3005416, NT2RI3005861, NT2RI3005928, NT2RI3006132, NT2RI3006171, NT2RI3006340, NT2RI3006376, NT2RI3006666, NT2RI3006796, NT2RI3007065, NT2RI3007095, NT2RI3007158, NT2RI3007167, NT2RI3007213, NT2RI3007443, NT2RI3007684, NT2RI3007757, NT2RI3007878, NT2RI3007978, NT2RI3008162, NT2RI3008179, NT2RI3008228, NT2RI3008442, NT2RI3008652, NT2RI3008697, NT2RI3008974, NT2RI3009158, NT2RI3009480, NT2RP6000005, NT2RP6000008, NT2RP6000032, NT2RP6000033, NT2RP6000059, NT2RP6000060, NT2RP6000086, NT2RP6000100, NT2RP6000119, NT2RP6000127, NT2RP6000130, NT2RP6000140, NT2RP6000150, NT2RP7000041, NT2RP7000086, NT2RP7000109, NT2RP7000112, NT2RP7000311, NT2RP7000359, NT2RP7000364, NT2RP7000380, NT2RP7000391, NT2RP7000425, NT2RP7000579, NT2RP7000812, NT2RP7000926, NT2RP7001074, NT2RP7001156, NT2RP7001319, NT2RP7001364, NT2RP7001475, NT2RP7001532, NT2RP7001591, NT2RP7001602, NT2RP7001678, NT2RP7001856, NT2RP7002028, NT2RP7002151, NT2RP7002163, NT2RP7002243, NT2RP7002282, NT2RP7002554, NT2RP7002603, NT2RP7002619, NT2RP7002650, NT2RP7002779, NT2RP7002829, NT2RP7002841, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003261, NT2RP7003439, NT2RP7003511, NT2RP7003724, NT2RP7004027, NT2RP7004080, NT2RP7004114, NT2RP7004260, NT2RP7004358, NT2RP7004396, NT2RP7004541, NT2RP7004641, NT2RP7004656, NT2RP7004687, NT2RP7004722, NT2RP7004766, NT2RP7004961, NT2RP7004964, NT2RP7004975, NT2RP7005118, NT2RP7005205, NT2RP7005219, NT2RP7005323, NT2RP7005468, NT2RP7005493, NT2RP7005629, NT2RP7005631, NT2RP7005669, NT2RP7005750, NT2RP7005846, NT2RP7006160, NT2RP7006162, NT2RP7006296, NT2RP7006304, NT2RP7006374, NT2RP7006408, NT2RP7006457, NT2RP7006547, NT2RP7006601, NT2RP7006621, NT2RP7006636, NT2RP7006853, NT2RP7006887, NT2RP7006980, NT2RP7006995, NT2RP7007114, NT2RP7007221, NT2RP7007226, NT2RP7007269, NT2RP7007310, NT2RP7007359, NT2RP7007406, NT2RP7007480, NT2RP7007504, NT2RP7007580, NT2RP7007594, NT2RP7007643, NT2RP7007766, NT2RP7007925, NT2RP7007930, NT2RP7008013, NT2RP7008015, NT2RP7008133, NT2RP7008137, NT2RP7008142, NT2RP7008144, NT2RP7008360, NT2RP7008406, NT2RP7008435, NT2RP7008441, NT2RP7008454, NT2RP7008544, NT2RP7008550, NT2RP7008557, NT2RP7008623, NT2RP7008657, NT2RP7008720, NT2RP7008855, NT2RP7008863, NT2RP7009019, NT2RP7009030, NT2RP7009097, NT2RP7009168, NT2RP7009215, NT2RP7009236, NT2RP7009253, NT2RP7009267, NT2RP7009322, NT2RP7009363, NT2RP7009370, NT2RP7009397, NT2RP7009429, NT2RP7009439, NT2RP7009466, NT2RP7009502, NT2RP7009577, NT2RP7009743, NT2RP7009867, NT2RP7010109, NT2RP7010128, NT2RP7010235, NT2RP7010275, NT2RP7010521, NT2RP7010599, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7013002, NT2RP7013374, NT2RP7013499, NT2RP7013729, NT2RP7013795, NT2RP7013999, NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015512, NT2RP7015676, NT2RP7015789, NT2RP7015996, NT2RP7016508, NT2RP7016622, NT2RP7016911, NT2RP7017139, NT2RP7017284, NT2RP7017365, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019273, NT2RP7019367, NT2RP7019445, NT2RP7019682, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001604, NT2RP8003490, NT2RP8003657, NT2RP8003787, NT2RP8004306, NT2RP8005546, NT2RP8006452, NT2RP8006521, NT2RP8007416, NT2RP8007503, NT2RP8008057, NT2RP8009248, NTONG1000006, NTONG1000047, NTONG1000052, NTONG1000075, NTONG1000087, NTONG1000123, NTONG1000130, NTONG1000161, NTONG1000172, NTONG1000213, NTONG1000214, NTONG1000246, NTONG1000247, NTONG1000257, NTONG2000040, NTONG2000231, NTONG2000334, NTONG2000363, NTONG2000413, NTONG2000492, NTONG2000499, NTONG2000531, NTONG2000583, NTONG2000800, NTONG2000858, NTONG2000966, NTONG2000985, NTONG2001014, NTONG2001079, NTONG2001157, NTONG2001173, NTONG2001220, NTONG2001222, NTONG2001362, NTONG2001532, NTONG2001587, NTONG2001619, NTONG2001642, NTONG2001657, NTONG2001676, NTONG2002278, NTONG2002582, NTONG2002948, NTONG2002970, NTONG2003158, NTONG2003210, NTONG2003316, NTONG2003409, NTONG2003805, NTONG2003839, NTONG2003852, NTONG2003897, NTONG2003928, NTONG2004308, NTONG2004521, NTONG2004690, NTONG2004806, NTONG2004829, NTONG2004844, NTONG2004918, NTONG2004991, NTONG2005062, NTONG2005086, NTONG2005137, NTONG2005153, NTONG2005265, NTONG2005277, NTONG2005363, NTONG2005480, NTONG2005497, NTONG2005577, NTONG2005657, NTONG2005822, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007034, NTONG2007249, NTONG2007517, NTONG2007522, NTONG2007693, NTONG2007756, NTONG2008093, NTONG2008365, NTONG2008522, NTONG2008672, NTONG2008862, NTONG2008939, NTONG2009060, NTONG2009068, NTONG2009229, NTONG2009233, NTONG2009468, OCBBF1000016, OCBBF1000042, OCBBF1000054, OCBBF1000067, OCBBF1000085, OCBBF1000091, OCBBF1000104, OCBBF1000118, OCBBF1000119, OCBBF1000122, OCBBF1000130, OCBBF1000138, OCBBF1000153, OCBBF1000175, OCBBF1000192, OCBBF1000254, OCBBF2000013, OCBBF2000287, OCBBF2000323, OCBBF2000452, OCBBF2000467, OCBBF2000677, OCBBF2000703, OCBBF2000719, OCBBF2000998, OCBBF2001075, OCBBF2001101, OCBBF2001124, OCBBF2001176, OCBBF2001186, OCBBF2001196, OCBBF2001252, OCBBF2001323, OCBBF2001389, OCBBF2001402, OCBBF2001473, OCBBF2001494, OCBBF2001527, OCBBF2001528, OCBBF2001681, OCBBF2001706, OCBBF2001749, OCBBF2001910, OCBBF2001961, OCBBF2001983, OCBBF2002124, OCBBF2002161, OCBBF2002357, OCBBF2002376, OCBBF2002615, OCBBF2002663, OCBBF2002805, OCBBF2002865, OCBBF2002920, OCBBF2003028, OCBBF2003091, OCBBF2003327, OCBBF2003543, OCBBF2003744, OCBBF2004038, OCBBF2004147, OCBBF2004168, OCBBF2004235, OCBBF2004385, OCBBF2004418, OCBBF2004478, OCBBF2004482, OCBBF2004567, OCBBF2004612, OCBBF2004647, OCBBF2004826, OCBBF2004866, OCBBF2004883, OCBBF2005066, OCBBF2005077, OCBBF2005343, OCBBF2005349, OCBBF2005373, OCBBF2005433, OCBBF2005476, OCBBF2005843, OCBBF2005901, OCBBF2006030, OCBBF2006058, OCBBF2006113, OCBBF2006154, OCBBF2006214, OCBBF2006241, OCBBF2006388, OCBBF2006438, OCBBF2006567, OCBBF2006624, OCBBF2006639, OCBBF2006849, OCBBF2006859, OCBBF2007039, OCBBF2007114, OCBBF2007121, OCBBF2007184, OCBBF2007194, OCBBF2007196, OCBBF2007238, OCBBF2007414, OCBBF2007428, OCBBF2007478, OCBBF2007485, OCBBF2007520, OCBBF2007555, OCBBF2007610, OCBBF2007622, OCBBF2007756, OCBBF2007931, OCBBF2007946, OCBBF2008116, OCBBF2008138, OCBBF2008283, OCBBF2008330, OCBBF2008366, OCBBF2008511, OCBBF2008520, OCBBF2008569, OCBBF2008586, OCBBF2008640, OCBBF2008691, OCBBF2008724, OCBBF2008760, OCBBF2008768, OCBBF2008770, OCBBF2008775, OCBBF2008790, OCBBF2008814, OCBBF2008822, OCBBF2009115, OCBBF2009391, OCBBF2009424, OCBBF2009571, OCBBF2009583, OCBBF2009603, OCBBF2009788, OCBBF2009926, OCBBF2010040, OCBBF2010281, OCBBF2010313, OCBBF2010404, OCBBF2010416, OCBBF2010420, OCBBF2010557, OCBBF2010604, OCBBF2010709, OCBBF2010792, OCBBF2010843, OCBBF2010863, OCBBF2010931, OCBBF2010945, OCBBF2010978, OCBBF2010989, OCBBF2011137, OCBBF2011177, OCBBF2011311, OCBBF2011625, OCBBF2011669, OCBBF2011685, OCBBF2011706, OCBBF2011722, OCBBF2011855, OCBBF2011872, OCBBF2011960, OCBBF2011981, OCBBF2012028, OCBBF2012139, OCBBF2012191, OCBBF2012320, OCBBF2012436, OCBBF2012525, OCBBF2012714, OCBBF2012755, OCBBF2012812, OCBBF2012821, OCBBF2012881, OCBBF2012990, OCBBF2013049, OCBBF2013091, OCBBF2013127, OCBBF2013208, OCBBF2013285, OCBBF2013319, OCBBF2013324, OCBBF2013507, OCBBF2013721, OCBBF2013883, OCBBF2013926, OCBBF2014052, OCBBF2014386, OCBBF2014707, OCBBF2014745, OCBBF2014828, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015506, OCBBF2015599, OCBBF2015645, OCBBF2015659, OCBBF2015751, OCBBF2015797, OCBBF2015980, OCBBF2016038, OCBBF2016405, OCBBF2016467, OCBBF2016590, OCBBF2016591, OCBBF2016612, OCBBF2016689, OCBBF2016690, OCBBF2016841, OCBBF2016928, OCBBF2017055, OCBBF2017069, OCBBF2017325, OCBBF2017458, OCBBF2017489, OCBBF2017516, OCBBF2017665, OCBBF2017815, OCBBF2018012, OCBBF2018014, OCBBF2018167, OCBBF2018229, OCBBF2018362, OCBBF2018380, OCBBF2018563, OCBBF2018663, OCBBF2018687, OCBBF2018828, OCBBF2018934, OCBBF2019108, OCBBF2019195, OCBBF2019327, OCBBF2019684, OCBBF2019761, OCBBF2019823, OCBBF2020048, OCBBF2020318, OCBBF2020343, OCBBF2020453, OCBBF2020801, OCBBF2020825, OCBBF2020838, OCBBF2021020, OCBBF2021214, OCBBF2021323, OCBBF2021518, OCBBF2021788, OCBBF2022351, OCBBF2022573, OCBBF2022574, OCBBF2023162, OCBBF2023598, OCBBF2023643, OCBBF2024463, OCBBF2024589, OCBBF2024779, OCBBF2024781, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028173, OCBBF2028421, OCBBF2029471, OCBBF2029901, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2032152, OCBBF2032274, OCBBF2032539, OCBBF2032590, OCBBF2032599, OCBBF2032611, OCBBF2032671, OCBBF2033413, OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF3000097, OCBBF3000213, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003209, OCBBF3003592, OCBBF3003745, OCBBF3003761, OCBBF3004264, OCBBF3004314, OCBBF3004972, OCBBF3005330, OCBBF3005597, OCBBF3005843, OCBBF3006802, OCBBF3006986, OCBBF3007078, OCBBF3007704, OCBBF3008392, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000147, PEBLM1000174, PEBLM1000180, PEBLM2000112, PEBLM2000126, PEBLM2000170, PEBLM2000180, PEBLM2000213, PEBLM2000248, PEBLM2000270, PEBLM2000321, PEBLM2000333, PEBLM2000338, PEBLM2000479, PEBLM2000502, PEBLM2001312, PEBLM2001465, PEBLM2001488, PEBLM2001803, PEBLM2002432, PEBLM2002594, PEBLM2002749, PEBLM2002887, PEBLM2003426, PEBLM2003765, PEBLM2003935, PEBLM2004015, PEBLM2004216, PEBLM2004290, PEBLM2004497, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006298, PEBLM2006709, PEBLM2006912, PEBLM2007112, PEBLM2007140, PEBLM2007188, PEBLM2007296, PEBLM2007437, PEBLM2007447, PEBLM2007598, PEBLM2007832, PEBLM2007834, PEBLM2008106, PEBLM2008576, PEBLM2008605, PEBLM2008861, PERIC1000025, PERIC1000147, PERIC2000180, PERIC2000214, PERIC2000386, PERIC2000387, PERIC2000422, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000037, PLACE5000067, PLACE5000068, PLACE5000070, PLACE5000080, PLACE5000105, PLACE5000115, PLACE5000116, PLACE5000129, PLACE5000139, PLACE5000153, PLACE5000171, PLACE5000245, PLACE5000260, PLACE5000282, PLACE5000372, PLACE5000522, PLACE5000527, PLACE6000012, PLACE6000044, PLACE6000055, PLACE6000070, PLACE6000137, PLACE6000145, PLACE6000191, PLACE6000205, PLACE6000231, PLACE6000296, PLACE6000348, PLACE6000371, PLACE6000424, PLACE6000426, PLACE6000429, PLACE6000450, PLACE6000523, PLACE6000550, PLACE6000552, PLACE6000555, PLACE6000619, PLACE6000630, PLACE6000953, PLACE6001118, PLACE6001153, PLACE6001262, PLACE6001294, PLACE6001536, PLACE6001557, PLACE6001712, PLACE6001740, PLACE6001886, PLACE6001923, PLACE6001925, PLACE6001933, PLACE6002011, PLACE6002016, PLACE6002056, PLACE6002157, PLACE6002312, PLACE6002323, PLACE6002345, PLACE6002419, PLACE6002462, PLACE6002647, PLACE6002692, PLACE6002699, PLACE6002710, PLACE6002871, PLACE6002949, PLACE6002960, PLACE6003038, PLACE6003077, PLACE6003094, PLACE6003109, PLACE6003372, PLACE6003393, PLACE6003399, PLACE6003539, PLACE6003621, PLACE6003705, PLACE6003740, PLACE6003771, PLACE6003796, PLACE6003839, PLACE6004101, PLACE6004219, PLACE6004336, PLACE6004368, PLACE6004380, PLACE6004396, PLACE6004454, PLACE6004464, PLACE6004491, PLACE6004663, PLACE6004687, PLACE6004738, PLACE6004931, PLACE6004966, PLACE6004993, PLACE6005007, PLACE6005029, PLACE6005108, PLACE6005234, PLACE6005283, PLACE6005328, PLACE6005351, PLACE6005423, PLACE6005487, PLACE6005535, PLACE6005546, PLACE6005559, PLACE6005579, PLACE6005691, PLACE6005786, PLACE6006042, PLACE6006077, PLACE6006158, PLACE6006217, PLACE6006266, PLACE6006287, PLACE6006394, PLACE6006474, PLACE6006549, PLACE6006566, PLACE6006697, PLACE6006822, PLACE6006871, PLACE6006905, PLACE6006988, PLACE6007050, PLACE6007239, PLACE6007242, PLACE6007380, PLACE6007433, PLACE6007482, PLACE6007687, PLACE6007787, PLACE6007798, PLACE6007807, PLACE6008036, PLACE6008094, PLACE6008126, PLACE6008285, PLACE6008444, PLACE6008453, PLACE6008640, PLACE6008750, PLACE6008768, PLACE6008793, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009835, PLACE6009853, PLACE6010463, PLACE6010485, PLACE6010568, PLACE6010701, PLACE6010765, PLACE6010848, PLACE6010925, PLACE6010936, PLACE6010991, PLACE6011041, PLACE6011102, PLACE6011211, PLACE6011260, PLACE6011334, PLACE6011627, PLACE6011774, PLACE6011881, PLACE6011907, PLACE6011970, PLACE6011975, PLACE6012108, PLACE6012168, PLACE6012297, PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013672, PLACE6013884, PLACE6013885, PLACE6014043, PLACE6014516, PLACE6014882, PLACE6015211, PLACE6015322, PLACE6015460, PLACE6015513, PLACE6015591, PLACE6015729, PLACE6015939, PLACE6016030, PLACE6016093, PLACE6016105, PLACE6016160, PLACE6016210, PLACE6016254, PLACE6016383, PLACE6017002, PLACE6017431, PLACE6017564, PLACE6017578, PLACE6017626, PLACE6017714, PLACE6017788, PLACE6017791, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6018938, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7005840, PLACE7006051, PLACE7006090, PLACE7006268, PLACE7006275, PLACE7006498, PLACE7006540, PLACE7007379, PLACE7007644, PLACE7007973, PLACE7008136, PLACE7008431, PLACE7008623, PLACE7008766, PLACE7009563, PROST1000033, PROST1000039, PROST1000065, PROST1000083, PROST1000110, PROST1000120, PROST1000123, PROST1000136, PROST1000145, PROST1000152, PROST1000167, PROST1000184, PROST1000215, PROST1000217, PROST1000220, PROST1000226, PROST1000246, PROST1000262, PROST1000272, PROST1000298, PROST1000322, PROST1000334, PROST1000343, PROST1000362, PROST1000377, PROST1000412, PROST1000419, PROST1000451, PROST1000480, PROST1000485, PROST1000493, PROST1000512, PROST1000527, PROST1000528, PROST1000536, PROST1000559, PROST1000575, PROST2000036, PROST2000053, PROST2000067, PROST2000079, PROST2000105, PROST2000143, PROST2000162, PROST2000165, PROST2000176, PROST2000203, PROST2000212, PROST2000241, PROST2000267, PROST2000273, PROST2000274, PROST2000277, PROST2000284, PROST2000325, PROST2000337, PROST2000432, PROST2000452, PROST2000470, PROST2000505, PROST2000567, PROST2000572, PROST2000717, PROST2000761, PROST2000835, PROST2000893, PROST2000961, PROST2001002, PROST2001116, PROST2001138, PROST2001213, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001823, PROST2001883, PROST2001899, PROST2001998, PROST2002078, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003302, PROST2003303, PROST2003340, PROST2003428, PROST2003472, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004332, PROST2004481, PROST2004526, PROST2004570, PROST2004727, PROST2004742, PROST2004744, PROST2004817, PROST2005039, PROST2005093, PROST2005121, PROST2005131, PROST2005143, PROST2005228, PROST2005285, PROST2005426, PROST2005466, PROST2005630, PROST2005683, PROST2005788, PROST2005880, PROST2005886, PROST2005904, PROST2005919, PROST2005943, PROST2006008, PROST2006020, PROST2006060, PROST2006196, PROST2006201, PROST2006282, PROST2006343, PROST2006450, PROST2006491, PROST2006510, PROST2006536, PROST2006579, PROST2006688, PROST2006737, PROST2006766, PROST2006782, PROST2006933, PROST2007122, PROST2007200, PROST2007237, PROST2007289, PROST2007317, PROST2007328, PROST2007382, PROST2007389, PROST2007416, PROST2007528, PROST2007818, PROST2007871, PROST2007950, PROST2007956, PROST2007974, PROST2007988, PROST2008079, PROST2008088, PROST2008243, PROST2008245, PROST2008268, PROST2008447, PROST2008468, PROST2008472, PROST2008516, PROST2008724, PROST2008835, PROST2008993, PROST2009022, PROST2009208, PROST2009222, PROST2009254, PROST2009273, PROST2009320, PROST2009365, PROST2009388, PROST2009400, PROST2009470, PROST2009483, PROST2009571, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010579, PROST2010606, PROST2010885, PROST2011012, PROST2011105, PROST2011297, PROST2011410, PROST2011439, PROST2011482, PROST2011631, PROST2011660, PROST2012005, PROST2012016, PROST2012088, PROST2012140, PROST2012157, PROST2012190, PROST2012249, PROST2012400, PROST2012504, PROST2012542, PROST2012550, PROST2012740, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015137, PROST2015198, PROST2015243, PROST2015246, PROST2015287, PROST2015457, PROST2015537, PROST2015545, PROST2015551, PROST2015636, PROST2015756, PROST2015877, PROST2015900, PROST2015924, PROST2015932, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016980, PROST2017098, PROST2017128, PROST2017203, PROST2017367, PROST2017413, PROST2017441, PROST2017578, PROST2017612, PROST2017617, PROST2017700, PROST2017701, PROST2017729, PROST2017836, PROST2017910, PROST2018030, PROST2018090, PROST2018235, PROST2018437, PROST2018487, PROST2018511, PROST2018607, PROST2018788, PROST2018902, PROST2018922, PROST2019296, PROST2019398, PROST2019487, PROST2019781, PUAEN1000039, PUAEN1000081, PUAEN1000085, PUAEN1000087, PUAEN1000121, PUAEN1000175, PUAEN1000275, PUAEN1000276, PUAEN1000288, PUAEN2000080, PUAEN2000175, PUAEN2000247, PUAEN2000394, PUAEN2000497, PUAEN2000594, PUAEN2000684, PUAEN2000942, PUAEN2001260, PUAEN2001526, PUAEN2001882, PUAEN2002473, PUAEN2002552, PUAEN2002568, PUAEN2002616, PUAEN2002758, PUAEN2003079, PUAEN2003116, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2006029, PUAEN2006328, PUAEN2006578, PUAEN2006639, PUAEN2006761, PUAEN2007350, PUAEN2007785, PUAEN2007898, PUAEN2008123, PUAEN2008228, PUAEN2008314, PUAEN2008815, PUAEN2008939, PUAEN2008980, PUAEN2009174, PUAEN2009348, PUAEN2009534, PUAEN2009795, PUAEN2009852, RECTM1000051, RECTM1000141, RECTM2000220, RECTM2000349, RECTM2000433, RECTM2000510, RECTM2001307, RECTM2001519, RECTM2001666, RECTM2001691, RECTM2001749, RECTM2002172, RECTM2002602, RECTM2002632, SALGL1000024, SALGL1000028, SALGL1000065, SALGL1000107, SALGL1000171, SKMUS1000030, SKMUS1000064, SKMUS1000067, SKMUS1000104, SKMUS1000118, SKMUS1000124, SKMUS1000129, SKMUS1000138, SKMUS2000061, SKMUS2000074, SKMUS2000078, SKMUS2000137, SKMUS2000198, SKMUS2000243, SKMUS2000287, SKMUS2000317, SKMUS2000330, SKMUS2000339, SKMUS2000343, SKMUS2000365, SKMUS2000390, SKMUS2000416, SKMUS2000458, SKMUS2000467, SKMUS2000468, SKMUS2000515, SKMUS2000679, SKMUS2000690, SKMUS2000701, SKMUS2000705, SKMUS2000710, SKMUS2000724, SKMUS2000726, SKMUS2000774, SKMUS2000780, SKMUS2000821, SKMUS2000847, SKMUS2000863, SKMUS2000894, SKMUS2000898, SKMUS2000902, SKMUS2000933, SKMUS2000945, SKMUS2000973, SKMUS2000980, SKMUS2001008, SKMUS2001014, SKMUS2001129, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001501, SKMUS2001543, SKMUS2001580, SKMUS2001585, SKMUS2001608, SKMUS2001631, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000123, SKNMC1000124, SKNMC1000137, SKNMC1000229, SKNMC1000251, SKNMC1000264, SKNMC2000224, SKNMC2000256, SKNMC2000356, SKNMC2000374, SKNMC2000583, SKNMC2000593, SKNMC2000612, SKNMC2000622, SKNMC2000698, SKNMC2000966, SKNMC2001113, SKNMC2001324, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006327, SKNMC2006998, SKNMC2007134, SKNMC2007502, SKNMC2007504, SKNMC2007961, SKNMC2009450, SKNSH1000086, SKNSH1000174, SKNSH1000301, SKNSH1000308, SKNSH1000416, SKNSH2000051, SKNSH2000151, SKNSH2000163, SKNSH2000319, SKNSH2000482, SKNSH2000516, SKNSH2000550, SKNSH2000971, SKNSH2001931, SKNSH2002054, SKNSH2002768, SKNSH2002866, SKNSH2003174, SKNSH2003422, SKNSH2003466, SKNSH2003483, SKNSH2003528, SKNSH2003633, SKNSH2004039, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009435, SKNSH2009991, SKNSH2010015, SMINT1000012, SMINT1000016, SMINT1000039, SMINT1000042, SMINT1000051, SMINT1000054, SMINT1000057, SMINT1000071, SMINT1000075, SMINT1000078, SMINT1000091, SMINT1000103, SMINT1000118, SMINT1000137, SMINT1000192, SMINT2000007, SMINT2000018, SMINT2000032, SMINT2000040, SMINT2000060, SMINT2000084, SMINT2000145, SMINT2000159, SMINT2000176, SMINT2000185, SMINT2000227, SMINT2000229, SMINT2000232, SMINT2000277, SMINT2000518, SMINT2000529, SMINT2000530, SMINT2000537, SMINT2000538, SMINT2000541, SMINT2000558, SMINT2000562, SMINT2000568, SMINT2000602, SMINT2000609, SMINT2000747, SMINT2000811, SMINT2000824, SMINT2000984, SMINT2001114, SMINT2001158, SMINT2001199, SMINT2001222, SMINT2001397, SMINT2001458, SMINT2001461, SMINT2001535, SMINT2001559, SMINT2001609, SMINT2001615, SMINT2001683, SMINT2001731, SMINT2001812, SMINT2001818, SMINT2002126, SMINT2002202, SMINT2002210, SMINT2002281, SMINT2002311, SMINT2002314, SMINT2002328, SMINT2002414, SMINT2002457, SMINT2002743, SMINT2002778, SMINT2002880, SMINT2002882, SMINT2002884, SMINT2002899, SMINT2002974, SMINT2003003, SMINT2003074, SMINT2003128, SMINT2003150, SMINT2003317, SMINT2003340, SMINT2003386, SMINT2003505, SMINT2003551, SMINT2003569, SMINT2003641, SMINT2003644, SMINT2003866, SMINT2004037, SMINT2004047, SMINT2004086, SMINT2004144, SMINT2004280, SMINT2004299, SMINT2004339, SMINT2004414, SMINT2004473, SMINT2004547, SMINT2004583, SMINT2004729, SMINT2004781, SMINT2004891, SMINT2004909, SMINT2004972, SMINT2004992, SMINT2005075, SMINT2005161, SMINT2005174, SMINT2005213, SMINT2005387, SMINT2005402, SMINT2005405, SMINT2005621, SMINT2005623, SMINT2005624, SMINT2005688, SMINT2005800, SMINT2006078, SMINT2006205, SMINT2006331, SMINT2006596, SMINT2006641, SMINT2006648, SMINT2006708, SMINT2006716, SMINT2006801, SMINT2007062, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008625, SMINT2008672, SMINT2008690, SMINT2008715, SMINT2008720, SMINT2008817, SMINT2008844, SMINT2008917, SMINT2009119, SMINT2009121, SMINT2009212, SMINT2009216, SMINT2009233, SMINT2009259, SMINT2009272, SMINT2009363, SMINT2009468, SMINT2009505, SMINT2009529, SMINT2009832, SMINT2009895, SMINT2009902, SMINT2009973, SMINT2010015, SMINT2010068, SMINT2010076, SMINT2010200, SMINT2010278, SMINT2010326, SMINT2010369, SMINT2010426, SMINT2010500, SMINT2010533, SMINT2010629, SMINT2010672, SMINT2010753, SMINT2010853, SMINT2010897, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012195, SMINT2012285, SMINT2012291, SMINT2012734, SMINT2012793, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016286, SMINT2016313, SMINT2016412, SMINT2016440, SMINT2016477, SMINT2016544, SMINT2016857, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000041, SPLEN1000056, SPLEN1000087, SPLEN1000089, SPLEN1000091, SPLEN1000134, SPLEN1000139, SPLEN1000143, SPLEN1000145, SPLEN1000156, SPLEN1000163, SPLEN1000178, SPLEN2000020, SPLEN2000047, SPLEN2000064, SPLEN2000072, SPLEN2000126, SPLEN2000143, SPLEN2000189, SPLEN2000205, SPLEN2000210, SPLEN2000242, SPLEN2000243, SPLEN2000247, SPLEN2000270, SPLEN2000307, SPLEN2000310, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000516, SPLEN2000541, SPLEN2000607, SPLEN2000630, SPLEN2000695, SPLEN2000839, SPLEN2001135, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001650, SPLEN2001689, SPLEN2001710, SPLEN2001761, SPLEN2001781, SPLEN2001881, SPLEN2001912, SPLEN2001945, SPLEN2002007, SPLEN2002133, SPLEN2002147, SPLEN2002166, SPLEN2002175, SPLEN2002223, SPLEN2002314, SPLEN2002343, SPLEN2002354, SPLEN2002385, SPLEN2002419, SPLEN2002451, SPLEN2002462, SPLEN2002467, SPLEN2002477, SPLEN2002662, SPLEN2002695, SPLEN2002707, SPLEN2002917, SPLEN2002957, SPLEN2003160, SPLEN2003204, SPLEN2003219, SPLEN2003297, SPLEN2003878, SPLEN2003918, SPLEN2003924, SPLEN2004060, SPLEN2004078, SPLEN2004124, SPLEN2004148, SPLEN2004181, SPLEN2004220, SPLEN2004346, SPLEN2004368, SPLEN2004611, SPLEN2004662, SPLEN2004844, SPLEN2005227, SPLEN2005272, SPLEN2005416, SPLEN2005450, SPLEN2005474, SPLEN2005727, SPLEN2005790, SPLEN2005818, SPLEN2005869, SPLEN2005939, SPLEN2006232, SPLEN2006268, SPLEN2006283, SPLEN2006325, SPLEN2006389, SPLEN2006425, SPLEN2006671, SPLEN2006701, SPLEN2006785, SPLEN2006875, SPLEN2007314, SPLEN2007326, SPLEN2007350, SPLEN2007388, SPLEN2007619, SPLEN2007653, SPLEN2007689, SPLEN2007695, SPLEN2007750, SPLEN2007794, SPLEN2007879, SPLEN2007926, SPLEN2007951, SPLEN2008007, SPLEN2008135, SPLEN2008164, SPLEN2008317, SPLEN2008496, SPLEN2008591, SPLEN2008786, SPLEN2009088, SPLEN2009163, SPLEN2009315, SPLEN2009340, SPLEN2009512, SPLEN2009513, SPLEN2009541, SPLEN2009548, SPLEN2009581, SPLEN2009733, SPLEN2009803, SPLEN2009884, SPLEN2009918, SPLEN2010004, SPLEN2010112, SPLEN2010119, SPLEN2010146, SPLEN2010187, SPLEN2010350, SPLEN2010395, SPLEN2010415, SPLEN2010447, SPLEN2010510, SPLEN2010530, SPLEN2010534, SPLEN2010588, SPLEN2010625, SPLEN2010800, SPLEN2010846, SPLEN2010847, SPLEN2010862, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012524, SPLEN2012525, SPLEN2012606, SPLEN2012611, SPLEN2012725, SPLEN2012743, SPLEN2012756, SPLEN2012846, SPLEN2012889, SPLEN2012961, SPLEN2013108, SPLEN2013195, SPLEN2013644, SPLEN2013648, SPLEN2013670, SPLEN2013673, SPLEN2013690, SPLEN2013714, SPLEN2013753, SPLEN2013821, SPLEN2013860, SPLEN2013910, SPLEN2013923, SPLEN2014041, SPLEN2014063, SPLEN2014073, SPLEN2014080, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014669, SPLEN2014739, SPLEN2014860, SPLEN2014865, SPLEN2014919, SPLEN2014920, SPLEN2014924, SPLEN2014937, SPLEN2014946, SPLEN2015031, SPLEN2015094, SPLEN2015121, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015815, SPLEN2015890, SPLEN2015891, SPLEN2015899, SPLEN2016045, SPLEN2016053, SPLEN2016069, SPLEN2016098, SPLEN2016135, SPLEN2016241, SPLEN2016250, SPLEN2016304, SPLEN2016356, SPLEN2016480, SPLEN2016531, SPLEN2016541, SPLEN2016552, SPLEN2016554, SPLEN2016602, SPLEN2016627, SPLEN2016678, SPLEN2016773, SPLEN2016781, SPLEN2016840, SPLEN2016863, SPLEN2016922, SPLEN2016973, SPLEN2017043, SPLEN2017104, SPLEN2017147, SPLEN2017187, SPLEN2017189, SPLEN2017212, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017999, SPLEN2018098, SPLEN2018112, SPLEN2018180, SPLEN2018181, SPLEN2018280, SPLEN2018285, SPLEN2018364, SPLEN2018395, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019257, SPLEN2019311, SPLEN2019323, SPLEN2019375, SPLEN2019379, SPLEN2019446, SPLEN2019471, SPLEN2019480, SPLEN2019505, SPLEN2019571, SPLEN2019575, SPLEN2019709, SPLEN2019793, SPLEN2019811, SPLEN2019839, SPLEN2019956, SPLEN2019985, SPLEN2020007, SPLEN2020153, SPLEN2020154, SPLEN2020166, SPLEN2020183, SPLEN2020223, SPLEN2020359, SPLEN2020427, SPLEN2020467, SPLEN2020512, SPLEN2020608, SPLEN2020777, SPLEN2020805, SPLEN2021057, SPLEN2021157, SPLEN2021231, SPLEN2021295, SPLEN2021298, SPLEN2021383, SPLEN2021417, SPLEN2021440, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024530, SPLEN2024571, SPLEN2024577, SPLEN2024617, SPLEN2024737, SPLEN2024956, SPLEN2024990, SPLEN2025012, SPLEN2025017, SPLEN2025039, SPLEN2025219, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031724, SPLEN2031780, SPLEN2032036, SPLEN2032112, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034551, SPLEN2034601, SPLEN2034678, SPLEN2034864, SPLEN2034934, SPLEN2035318, SPLEN2035615, SPLEN2036103, SPLEN2036326, SPLEN2036475, SPLEN2036501, SPLEN2036579, SPLEN2036608, SPLEN2036702, SPLEN2036712, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039672, SPLEN2039697, SPLEN2039845, SPLEN2039936, SPLEN2040222, SPLEN2040237, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042598, SPLEN2042714, SPLEN2042920, STOMA1000013, STOMA1000047, STOMA1000052, STOMA1000074, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000121, STOMA2000148, STOMA2000168, STOMA2000183, STOMA2000188, STOMA2000279, STOMA2000383, STOMA2000386, STOMA2000395, STOMA2000478, STOMA2000529, STOMA2000539, STOMA2000567, STOMA2000640, STOMA2000678, STOMA2000686, STOMA2000888, STOMA2001025, STOMA2001389, STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005664, STOMA2005667, STOMA2005746, STOMA2005782, STOMA2006181, STOMA2006213, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, SYNOV1000045, SYNOV1000049, SYNOV1000118, SYNOV1000164, SYNOV1000190, SYNOV1000256, SYNOV2000152, SYNOV2000171, SYNOV2000173, SYNOV2000177, SYNOV2000178, SYNOV2000193, SYNOV2000251, SYNOV2000279, SYNOV2000297, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001212, SYNOV2001251, SYNOV2001300, SYNOV2001356, SYNOV2001374, SYNOV2001390, SYNOV2001451, SYNOV2001457, SYNOV2001468, SYNOV2001660, SYNOV2003326, SYNOV2005216, SYNOV2005448, SYNOV2005541, SYNOV2005673, SYNOV2005817, SYNOV2005962, SYNOV2006430, SYNOV2007965, SYNOV2008765, SYNOV2009172, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000472, SYNOV4000598, SYNOV4000706, SYNOV4001224, SYNOV4001326, SYNOV4001342, SYNOV4001395, SYNOV4002001, SYNOV4002392, SYNOV4002875, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4006256, SYNOV4006327, SYNOV4007012, SYNOV4007215, SYNOV4007360, SYNOV4007521, SYNOV4007553, SYNOV4007711, SYNOV4008336, SYNOV4009139, SYNOV4009298, T1ESE2000116, T1ESE2000609, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN2000139, TCOLN2000236, TCOLN2000641, TCOLN2001268, TCOLN2002047, TCOLN2002278, TESOP1000127, TESOP1000160, TESOP2000083, TESOP2000090, TESOP2000205, TESOP2000312, TESOP2000390, TESOP2000400, TESOP2000510, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002273, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000055, TESTI1000064, TESTI1000085, TESTI1000088, TESTI1000093, TESTI1000109, TESTI1000125, TESTI1000127, TESTI1000128, TESTI1000131, TESTI1000138, TESTI1000142, TESTI1000157, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000018, TESTI2000044, TESTI2000100, TESTI2000117, TESTI2000120, TESTI2000147, TESTI2000154, TESTI2000179, TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000443, TESTI2000452, TESTI2000462, TESTI2000571, TESTI2000591, TESTI2000598, TESTI2000616, TESTI2000644, TESTI2000671, TESTI2000683, TESTI2000689, TESTI2000695, TESTI2000699, TESTI2000707, TESTI2000745, TESTI2000756, TESTI2000762, TESTI2000808, TESTI2000819, TESTI2000830, TESTI2000840, TESTI2000850, TESTI2000909, TESTI2000951, TESTI2000981, TESTI2001008, TESTI2001049, TESTI2001082, TESTI2001134, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001879, TESTI2001894, TESTI2001915, TESTI2001950, TESTI2001959, TESTI2001968, TESTI2001991, TESTI2002002, TESTI2002012, TESTI2002036, TESTI2002048, TESTI2002057, TESTI2002081, TESTI2002090, TESTI2002105, TESTI2002149, TESTI2002216, TESTI2002223, TESTI2002245, TESTI2002251, TESTI2002264, TESTI2002282, TESTI2002293, TESTI2002294, TESTI2002326, TESTI2002351, TESTI2002361, TESTI2002369, TESTI2002406, TESTI2002415, TESTI2002423, TESTI2002453, TESTI2002461, TESTI2002465, TESTI2002498, TESTI2002516, TESTI2002544, TESTI2002578, TESTI2002580, TESTI2002618, TESTI2002676, TESTI2002690, TESTI2002696, TESTI2002698, TESTI2002700, TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003152, TESTI2003181, TESTI2003193, TESTI2003196, TESTI2003228, TESTI2003236, TESTI2003255, TESTI2003258, TESTI2003277, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003418, TESTI2003419, TESTI2003432, TESTI2003475, TESTI2003476, TESTI2003498, TESTI2003512, TESTI2003539, TESTI2003545, TESTI2003551, TESTI2003574, TESTI2003579, TESTI2003580, TESTI2003589, TESTI2003598, TESTI2003615, TESTI2003638, TESTI2003649, TESTI2003718, TESTI2003727, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004085, TESTI2004089, TESTI2004111, TESTI2004119, TESTI2004122, TESTI2004163, TESTI2004169, TESTI2004207, TESTI2004215, TESTI2004227, TESTI2004229, TESTI2004243, TESTI2004255, TESTI2004287, TESTI2004313, TESTI2004318, TESTI2004322, TESTI2004391, TESTI2004399, TESTI2004423, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004783, TESTI2004791, TESTI2004793, TESTI2004860, TESTI2004906, TESTI2004920, TESTI2004936, TESTI2004941, TESTI2004971, TESTI2004982, TESTI2004993, TESTI2004994, TESTI2004999, TESTI2005017, TESTI2005025, TESTI2005040, TESTI2005060, TESTI2005072, TESTI2005085, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415, TESTI2005470, TESTI2005492, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005739, TESTI2005767, TESTI2005775, TESTI2005827, TESTI2005860, TESTI2005892, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006120, TESTI2006125, TESTI2006179, TESTI2006209, TESTI2006210, TESTI2006212, TESTI2006218, TESTI2006238, TESTI2006255, TESTI2006278, TESTI2006333, TESTI2006341, TESTI2006360, TESTI2006425, TESTI2006437, TESTI2006457, TESTI2006460, TESTI2006465, TESTI2006483, TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006774, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007220, TESTI2007234, TESTI2007320, TESTI2007346, TESTI2007358, TESTI2007402, TESTI2007405, TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI20094620 TESTI2009474, TESTI2009477, TESTI2009497, TESTI2009502, TESTI2009511, TESTI2009544, TESTI2009545, TESTI2009551, TESTI2009585, TESTI2009588, TESTI2009683, TESTI2009727, TESTI2009739, TESTI2009812, TESTI2009835, TESTI2009853, TESTI2009935, TESTI2009977, TESTI2010009, TESTI2010154, TESTI2010239, TESTI2010280, TESTI2010354, TESTI2010396, TESTI2010400, TESTI2010409, TESTI2010572, TESTI2010587, TESTI2010591, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011612, TESTI2011665, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012592, TESTI2012617, TESTI2012628, TESTI2012778, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013257, TESTI2013264, TESTI2013268, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014800, TESTI2014843, TESTI2014979, TESTI2014988, TESTI2015105, TESTI2015180, TESTI2015246, TESTI2015249, TESTI2015327, TESTI2015331, TESTI2015335, TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015987, TESTI2016033, TESTI2016046, TESTI2016101, TESTI2016162, TESTI2016197, TESTI2016298, TESTI2016331, TESTI2016337, TESTI2016410, TESTI2016421, TESTI2016509, TESTI2016568, TESTI2016599, TESTI2016629, TESTI2016663, TESTI2016667, TESTI2016688, TESTI2016701, TESTI2016758, TESTI2016848, TESTI2016863, TESTI2016888, TESTI2016922, TESTI2016942, TESTI2016950, TESTI2016996, TESTI2017017, TESTI2017028, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017727, TESTI2017740, TESTI2017775, TESTI2017816, TESTI2017832, TESTI2017923, TESTI2017932, TESTI2017951, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018428, TESTI2018475, TESTI2018482, TESTI2018521, TESTI2018565, TESTI2018581, TESTI2018687, TESTI2018697, TESTI2018838, TESTI2018867, TESTI2018941, TESTI2018957, TESTI2018974, TESTI2018975, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020981, TESTI2020999, TESTI2021003, TESTI2021057, TESTI2021077, TESTI2021112, TESTI2021114, TESTI2021116, TESTI2021118, TESTI2021122, TESTI2021138, TESTI2021315, TESTI2021447, TESTI2021463, TESTI2021599, TESTI2021637, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024641, TESTI2024648, TESTI2024718, TESTI2024744, TESTI2024844, TESTI2024864, TESTI2024885, TESTI2024936, TESTI2024978, TESTI2024999, TESTI2025006, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025486, TESTI2025546, TESTI2025582, TESTI2025609, TESTI2025656, TESTI2025791, TESTI2025872, TESTI2025879, TESTI2025911, TESTI2025920, TESTI2025924, TESTI2026014, TESTI2026024, TESTI2026064, TESTI2026077, TESTI2026104, TESTI2026134, TESTI2026168, TESTI2026196, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029244, TESTI2029249, TESTI2029252, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034931, TESTI2034953, TESTI2034997, TESTI2035003, TESTI2035078, TESTI2035084, TESTI2035107, TESTI2035121, TESTI2035124, TESTI2035183, TESTI2035262, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036736, TESTI2036822, TESTI2036833, TESTI2036913, TESTI2036922, TESTI2036951, TESTI2036965, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039209, TESTI2039225, TESTI2039227, TESTI2039276, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001473, TESTI4001517, TESTI4001527, TESTI4001561, TESTI4001665, TESTI4001679, TESTI4001795, TESTI4001923, TESTI4001925, TESTI4002003, TESTI4002141, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003179, TESTI4003279, TESTI4003319, TESTI4003404, TESTI4003565, TESTI4003574, TESTI4003579, TESTI4003733, TESTI4003796, TESTI4004003, TESTI4004031, TESTI4004200, TESTI4004539, TESTI4004653, TESTI4004722, TESTI4004887, TESTI4004917, TESTI4005015, TESTI4005039, TESTI4005158, TESTI4005317, TESTI4005322, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006148, TESTI4006219, TESTI4006234, TESTI4006308, TESTI4006326, TESTI4006393, TESTI4006412, TESTI4006420, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4008007, TESTI4008018, TESTI4008050, TESTI4008086, TESTI4008176, TESTI4008209, TESTI4008219, TESTI4008250, TESTI4008302, TESTI4008305, TESTI4008401, TESTI4008417, TESTI4008429, TESTI4008573, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010475, TESTI4010544, TESTI4010713, TESTI4010721, TESTI4010789, TESTI4010817, TESTI4010831, TESTI4010902, TESTI4011070, TESTI4011072, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012505, TESTI4012556, TESTI4012623, TESTI4012679, TESTI4012911, TESTI4012956, TESTI4012960, TESTI4013365, TESTI4013369, TESTI4013410, TESTI4013602, TESTI4013667, TESTI4013675, TESTI4013685, TESTI4013735, TESTI4013742, TESTI4013774, TESTI4013830, TESTI4013852, TESTI4013924, TESTI4013960, TESTI4013962, TESTI4014175, TESTI4014265, TESTI4014276, TESTI4014306, TESTI4014392, TESTI4014415, TESTI4014445, TESTI4014553, TESTI4014661, TESTI4014801, TESTI4014891, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024494, TESTI4024740, TESTI4024874, TESTI4024890, TESTI4024907, TESTI4024930, TESTI4025062, TESTI4025401, TESTI4025494, TESTI4025731, TESTI4025797, TESTI4025865, TESTI4025908, TESTI4025920, TESTI4026079, TESTI4026080, TESTI4026192, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032112, TESTI4032128, TESTI4032270, TESTI4032375, TESTI4032834, TESTI4032856, TESTI4032895, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044770, TESTI4045168, TESTI4045312, TESTI4045330, TESTI4045470, TESTI4045701, TESTI4045908, TESTI4045955, TESTI4045982, TESTI4046090, TESTI4046240, TESTI4046245, TESTI4046253, TESTI4046282, TESTI4046328, TESTI4046450, TESTI4046457, TESTI4046487, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000017, THYMU1000025, THYMU1000032, THYMU1000033, THYMU1000060, THYMU1000074, THYMU1000098, THYMU1000136, THYMU1000142, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000331, THYMU1000336, THYMU1000366, THYMU1000382, THYMU1000393, THYMU1000394, THYMU1000399, THYMU1000426, THYMU1000428, THYMU1000440, THYMU1000459, THYMU1000473, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000120, THYMU2000140, THYMU2000162, THYMU2000171, THYMU2000176, THYMU2000179, THYMU2000231, THYMU2000276, THYMU2000280, THYMU2000300, THYMU2000317, THYMU2000368, THYMU2000369, THYMU2000418, THYMU2000440, THYMU2000489, THYMU2000492, THYMU2000519, THYMU2000570, THYMU2000602, THYMU2000660, THYMU2000671, THYMU2000672, THYMU2000702, THYMU2000767, THYMU2000800, THYMU2000879, THYMU2000932, THYMU2000950, THYMU2000982, THYMU2000990, THYMU2001053, THYMU2001071, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001443, THYMU2001521, THYMU2001556, THYMU2001622, THYMU2001727, THYMU2001819, THYMU2001825, THYMU2001839, THYMU2001898, THYMU2001900, THYMU2001916, THYMU2001926, THYMU2001989, THYMU2002023, THYMU2002037, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002841, THYMU2002852, THYMU2002910, THYMU2002950, THYMU2002983, THYMU2003069, THYMU2003107, THYMU2003124, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003470, THYMU2003479, THYMU2003499, THYMU2003632, THYMU2003650, THYMU2003700, THYMU2003760, THYMU2003803, THYMU2003855, THYMU2004003, THYMU2004008, THYMU2004042, THYMU2004108, THYMU2004111, THYMU2004123, THYMU2004139, THYMU2004169, THYMU2004284, THYMU2004320, THYMU2004336, THYMU2004356, THYMU2004508, THYMU2004512, THYMU2004639, THYMU2004668, THYMU2004677, THYMU2004688, THYMU2004693, THYMU2004835, THYMU2004843, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2006001, THYMU20060.48, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006666, THYMU2006712, THYMU2006913, THYMU2006946, THYMU2006965, THYMU2007025, THYMU2007060, THYMU2007065, THYMU2007095, THYMU2007146, THYMU2007179, THYMU2007258, THYMU2007307, THYMU2007308, THYMU2007339, THYMU2007350, THYMU2007384, THYMU2007415, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007802, THYMU2007824, THYMU2007854, THYMU2007952, THYMU2007969, THYMU2008036, THYMU2008072, THYMU2008111, THYMU2008145, THYMU2008185, THYMU2008191, THYMU2008207, THYMU2008226, THYMU2008282, THYMU2008339, THYMU2008350, THYMU2008351, THYMU2008383, THYMU2008452, THYMU2008643, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010046, THYMU2010059, THYMU2010082, THYMU2010094, THYMU2010161, THYMU2010448, THYMU2010519, THYMU2010637, THYMU2010671, THYMU2010699, THYMU2010705, THYMU2010779, THYMU2010816, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012073, THYMU2012113, THYMU2012273, THYMU2012401, THYMU2012492, THYMU2012507, THYMU2012565, THYMU2012574, THYMU2012631, THYMU2012665, THYMU2012690, THYMU2012701, THYMU2012807, THYMU2012882, THYMU2012891, THYMU2012902, THYMU2012919, THYMU2013023, THYMU2013047, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015775, THYMU2015825, THYMU2015943, THYMU2015984, THYMU2016113, THYMU2016164, THYMU2016204, THYMU2016219, THYMU2016360, THYMU2016496, THYMU2016523, THYMU2016840, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023711, THYMU2023900, THYMU2024071, THYMU2024121, THYMU2024185, THYMU2024207, THYMU2024616, THYMU2024684, THYMU2024748, THYMU2025042, THYMU2025189, THYMU2025406, THYMU2025557, THYMU2025572, THYMU2025849, THYMU2025909, THYMU2025939, THYMU2026182, THYMU2026350, THYMU2026530, THYMU2026531, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031368, THYMU2031579, THYMU2031847, THYMU2031994, THYMU2032014, THYMU2032035, THYMU2032080, THYMU2032150, THYMU2032155, THYMU2032358, THYMU2032437, THYMU2032601, THYMU2032655, THYMU2032715, THYMU2032732, THYMU2032976, THYMU2033053, THYMU2033059, THYMU2033070, THYMU2033079, THYMU2033085, THYMU2033104, THYMU2033227, THYMU2033401, THYMU2033583, THYMU2033755, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2036058, THYMU2036105, THYMU2036207, THYMU2036252, THYMU2036265, THYMU2036366, THYMU2036459, THYMU2036604, THYMU2036653, THYMU2037081, THYMU2037208, THYMU2037233, THYMU2037234, THYMU2037348, THYMU2037406, THYMU2037965, THYMU2038058, THYMU2038189, THYMU2038199, THYMU2038301, THYMU2038369, THYMU2038389, THYMU2038615, THYMU2038636, THYMU2038739, THYMU2038772, THYMU2038797, THYMU2038906, THYMU2039305, THYMU2039334, THYMU2039350, THYMU2039411, THYMU2039719, THYMU2039780, THYMU2039788, THYMU2039989, THYMU2040029, THYMU2040110, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000390, THYMU3000490, THYMU3000504, THYMU3000542, THYMU3000655, THYMU3000826, THYMU3000841, THYMU3001077, THYMU3001082, THYMU3001083, THYMU3001133, THYMU3001151, THYMU3001472, THYMU3001593, THYMU3001883, THYMU3001939, THYMU3001991, THYMU3002452, THYMU3002578, THYMU3002661, THYMU3003092, THYMU3003212, THYMU3003309, THYMU3003403, THYMU3003565, THYMU3003763, THYMU3003865, THYMU3003958, THYMU3004157, THYMU3004628, THYMU3004632, THYMU3004866, THYMU3005050, THYMU3005378, THYMU3005407, THYMU3005415, THYMU3005482, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007368, THYMU3007559, THYMU3007845, THYMU3008060, THYMU3008136, THYMU3008216, THYMU3008436, THYMU3008672, THYMU3008883, THYMU3008935, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3033402, THYMU3033626, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037836, THYMU3037856, THYMU3037867, THYMU3037909, THYMU3037980, THYMU3038158, THYMU3038167, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046360, THYMU3046856, THYMU3047115, THYMU3047144, THYMU3047156, THYMU3047366, THYMU3047513, THYMU3047542, THYMU3047760, THYMU3047891, TKIDN1000062, TKIDN1000066, TKIDN1000171, TKIDN2000127, TKIDN2000240, TKIDN2000464, TKIDN2000701, TKIDN2001529, TKIDN2002329, TKIDN2002424, TKIDN2002632, TKIDN2003044, TKIDN2003062, TKIDN2003366, TKIDN2003396, TKIDN2003413, TKIDN2003601, TKIDN2004185, TKIDN2004386, TKIDN2004458, TKIDN2004748, TKIDN2005487, TKIDN2005721, TKIDN2005934, TKIDN2005947, TKIDN2005956, TKIDN2006525, TKIDN2006761, TKIDN2006852, TKIDN2007667, TKIDN2008176, TKIDN2008778, TKIDN2009092, TKIDN2009481, TKIDN2010200, TKIDN2010232, TKIDN2010278, TKIDN2010602, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014757, TKIDN2014771, TKIDN2015025, TKIDN2015161, TKIDN2015263, TKIDN2015285, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001616, TLIVE2001684, TLIVE2001828, TLIVE2001927, TLIVE2002336, TLIVE2002338, TLIVE2002562, TLIVE2002690, TLIVE2002882, TLIVE2003197, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2001281, TOVAR2001556, TOVAR2001730, TOVAR2002247, TOVAR2002514, TOVAR2002549, TOVAR2002800, TRACH1000038, TRACH1000057, TRACH1000063, TRACH1000074, TRACH1000100, TRACH1000106, TRACH1000125, TRACH1000140, TRACH1000144, TRACH1000212, TRACH1000240, TRACH2000002, TRACH2000015, TRACH2000024, TRACH2000032, TRACH2000081, TRACH2000083, TRACH2000148, TRACH2000185, TRACH2000196, TRACH2000235, TRACH2000243, TRACH2000250, TRACH2000259, TRACH2000272, TRACH2000282, TRACH2000287, TRACH2000289, TRACH2000306, TRACH2000312, TRACH2000336, TRACH2000411, TRACH2000496, TRACH2000497, TRACH2000522, TRACH2000559, TRACH2000660, TRACH2000665, TRACH2000675, TRACH2000677, TRACH2000702, TRACH2000703, TRACH2000767, TRACH2000775, TRACH2000807, TRACH2000862, TRACH2000873, TRACH2000894, TRACH2000898, TRACH2000923, TRACH2000944, TRACH2000981, TRACH2000986, TRACH2001037, TRACH2001060, TRACH2001101, TRACH2001121, TRACH2001147, TRACH2001154, TRACH2001188, TRACH2001192, TRACH2001249, TRACH2001275, TRACH2001335, TRACH2001347, TRACH2001395, TRACH2001400, TRACH2001403, TRACH2001416, TRACH2001443, TRACH2001474, TRACH2001523, TRACH2001525, TRACH2001532, TRACH2001592, TRACH2001596, TRACH2001621, TRACH2001651, TRACH2001684, TRACH2001933, TRACH2001968, TRACH2002043, TRACH2002054, TRACH2002092, TRACH2002138, TRACH2002784, TRACH2002840, TRACH2002847, TRACH2002853, TRACH2002862, TRACH2002954, TRACH2002960, TRACH2002988, TRACH2003070, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005666, TRACH2005698, TRACH2005710, TRACH2005720, TRACH2005769, TRACH2005800, TRACH2006015, TRACH2006097, TRACH2006194, TRACH2006327, TRACH2006331, TRACH2006387, TRACH2006528, TRACH2006670, TRACH2006762, TRACH2006866, TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007754, TRACH2007816, TRACH2007894, TRACH2007988, TRACH2008081, TRACH2008112, TRACH2008127, TRACH2008130, TRACH2008278, TRACH2008540, TRACH2008583, TRACH2008597, TRACH2008820, TRACH2008921, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011290, TRACH2011293, TRACH2011302, TRACH2011574, TRACH2011956, TRACH2012000, TRACH2012138, TRACH2012242, TRACH2012370, TRACH2012387, TRACH2012536, TRACH2012562, TRACH2012742, TRACH2012823, TRACH2012918, TRACH2013081, TRACH2013123, TRACH2013265, TRACH2013369, TRACH2013450, TRACH2013495, TRACH2013671, TRACH2013726, TRACH2013902, TRACH2013928, TRACH2013982, TRACH2013991, TRACH2014018, TRACH2014045, TRACH2014046, TRACH2014082, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2015180, TRACH2015243, TRACH2015316, TRACH2015381, TRACH2015486, TRACH2015654, TRACH2015824, TRACH2015987, TRACH2016080, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018084, TRACH2018262, TRACH2018273, TRACH2018278, TRACH2018345, TRACH2018380, TRACH2018449, TRACH2018512, TRACH2018646, TRACH2018663, TRACH2018718, TRACH2018835, TRACH2018882, TRACH2018914, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911, TRACH2025932, TRACH3000017, TRACH3000043, TRACH3000180, TRACH3000342, TRACH3000420, TRACH3000515, TRACH3000530, TRACH3000548, TRACH3000558, TRACH3000586, TRACH3000692, TRACH3000892, TRACH3000926, TRACH3001119, TRACH3001443, TRACH3001759, TRACH3002064, TRACH3002107, TRACH3002168, TRACH3002188, TRACH3002245, TRACH3002293, TRACH3002533, TRACH3002561, TRACH3002567, TRACH3002650, TRACH3002866, TRACH3002871, TRACH3002876, TRACH3002890, TRACH3003009, TRACH3003357, TRACH3003379, TRACH3003586, TRACH3003683, TRACH3003780, TRACH3003805, TRACH3003832, TRACH3004113, TRACH3004288, TRACH3004303, TRACH3004412, TRACH3004456, TRACH3004537, TRACH3004596, TRACH3004651, TRACH3004652, TRACH3004671, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006800, TRACH3006889, TRACH3006985, TRACH3007097, TRACH3007268, TRACH3007274, TRACH3007277, TRACH3007391, TRACH3007541, TRACH3007595, TRACH3007627, TRACH3007766, TRACH3007866, TRACH3007995, TRACH3008042, TRACH3008061, TRACH3008535, TRACH3008629, TRACH3008688, TRACH3008697, TRACH3008713, TRACH3008887, TRACH3008902, TRACH3008990, TRACH3009059, TRACH3009061, TRACH3009701, TRACH3009715, TRACH3009894, TRACH3009956, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024423, TRACH3024428, TRACH3024512, TRACH3024823, TRACH3025302, TRACH3025346, TRACH3025520, TRACH3026148, TRACH3026283, TRACH3026299, TRACH3026542, TRACH3026650, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036997, TRACH3037063, TRACH3037267, TRACH3037288, TRACH3037505, TRACH3037573, TRACH3037696, TRACH3037897, TRACH3038086, TSTOM1000186, TSTOM2000139, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER2000057, TUTER2000283, TUTER2000425, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000156, UMVEN2000046, UMVEN2000069, UTERU1000008, UTERU1000015, UTERU1000024, UTERU1000031, UTERU1000032, UTERU1000057, UTERU1000065, UTERU1000077, UTERU1000093, UTERU1000109, UTERU1000131, UTERU1000138, UTERU1000148, UTERU1000160, UTERU1000182, UTERU1000183, UTERU1000187, UTERU1000192, UTERU1000249, UTERU1000337, UTERU1000339, UTERU2000047, UTERU2000074, UTERU2000099, UTERU2000154, UTERU2000197, UTERU2000218, UTERU2000238, UTERU2000243, UTERU2000263, UTERU2000300, UTERU2000338, UTERU2000349, UTERU2000377, UTERU2000393, UTERU2000465, UTERU2000485, UTERU2000517, UTERU2000524, UTERU2000537, UTERU2000539, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2001024, UTERU2001176, UTERU2001281, UTERU2001389, UTERU2001409, UTERU2001412, UTERU2001504, UTERU2001747, UTERU2001876, UTERU2002001, UTERU2002011, UTERU2002176, UTERU2002198, UTERU2002332, UTERU2002473, UTERU2002547, UTERU2002693, UTERU2002733, UTERU2002736, UTERU2002737, UTERU2002826, UTERU2002841, UTERU2002993, UTERU2003035, UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004039, UTERU2004061, UTERU2004073, UTERU2004163, UTERU2004197, UTERU2004299, UTERU2004461, UTERU2004520, UTERU2004564, UTERU2004688, UTERU2004698, UTERU2004861, UTERU2005004, UTERU2005050, UTERU2005069, UTERU2005179, UTERU2005292, UTERU2005346, UTERU2005446, UTERU2005449, UTERU2005450, UTERU2005533, UTERU2005822, UTERU2005903, UTERU2005905, UTERU2006103, UTERU2006137, UTERU2006182, UTERU2006400, UTERU2006412, UTERU2006429, UTERU2006568, UTERU2006651, UTERU2006705, UTERU2006899, UTERU2007075, UTERU2007081, UTERU2007128, UTERU2007267, UTERU2007444, UTERU2007499, UTERU2007639, UTERU2007724, UTERU2007942, UTERU2008019, UTERU2008027, UTERU2008077, UTERU2008347, UTERU2008516, UTERU2008561, UTERU2008653, UTERU2008705, UTERU2008707, UTERU2008785, UTERU2008845, UTERU2008901, UTERU2008930, UTERU2008962, UTERU2009094, UTERU2009120, UTERU2009131, UTERU2009147, UTERU2009206, UTERU2009283, UTERU2009414, UTERU2009483, UTERU2009510, UTERU2009538, UTERU2009540, UTERU2009776, UTERU2009904, UTERU2009951, UTERU2010115, UTERU2010124, UTERU2010164, UTERU2010226, UTERU2010231, UTERU2010304, UTERU2010431, UTERU2010525, UTERU2010651, UTERU2010724, UTERU2010747, UTERU2011195, UTERU2011199, UTERU2011220, UTERU2011261, UTERU2011410, UTERU2011574, UTERU2011657, UTERU2011741, UTERU2011811, UTERU2011897, UTERU2011962, UTERU2011968, UTERU2012101, UTERU2012114, UTERU2012252, UTERU2012333, UTERU2012407, UTERU2012526, UTERU2012581, UTERU2012610, UTERU2012615, UTERU2012688, UTERU2012703, UTERU2012741, UTERU2012767, UTERU2012786, UTERU2012856, UTERU2012890, UTERU2012938, UTERU2012976, UTERU2013048, UTERU2013231, UTERU2013262, UTERU2013280, UTERU2013322, UTERU2013483, UTERU2013491, UTERU2013502, UTERU2013926, UTERU2013976, UTERU2014001, UTERU2014223, UTERU2014398, UTERU2014548, UTERU2014631, UTERU2014668, UTERU2014678, UTERU2014728, UTERU2014998, UTERU2015062, UTERU2015087, UTERU2015190, UTERU2015198, UTERU2015202, UTERU2015405, UTERU2015640, UTERU2015830, UTERU2015880, UTERU2016147, UTERU2016157, UTERU2016464, UTERU2016669, UTERU2016761, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018333, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019038, UTERU2019163, UTERU2019257, UTERU2019453, UTERU2019491, UTERU2019534, UTERU2019681, UTERU2019710, UTERU2019959, UTERU2019964, UTERU2020226, UTERU2020242, UTERU2020292, UTERU2020351, UTERU2020491, UTERU2020718, UTERU2021380, UTERU2021649, UTERU2022020, UTERU2022773, UTERU2022955, UTERU2022981, UTERU2023045, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026775, UTERU2027023, UTERU2027369, UTERU2027591, UTERU2027616, UTERU2027941, UTERU2028377, UTERU2029503, UTERU2029660, UTERU2029742, UTERU2029953, UTERU2030270, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000402, UTERU3000645, UTERU3000665, UTERU3000670, UTERU3000727, UTERU3000738, UTERU3000828, UTERU3000844, UTERU3000959, UTERU3001029, UTERU3001053, UTERU3001059, UTERU3001240, UTERU3001394, UTERU3001542, UTERU3001558, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003178, UTERU3003465, UTERU3003495, UTERU3003523, UTERU3003774, UTERU3003776, UTERU3004477, UTERU3004523, UTERU3004616, UTERU3004635, UTERU3004938, UTERU3004992, UTERU3005049, UTERU3005205, UTERU3005264, UTERU3005460, UTERU3005536, UTERU3005585, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007731, UTERU3007913, UTERU3008463, UTERU3008660, UTERU3008671, UTERU3008722, UTERU3008917, UTERU3009490, UTERU3009517, UTERU3009690, UTERU3009775, UTERU3009979, UTERU3009987, UTERU3010029, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012476, UTERU3012599, UTERU3012999, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016274, UTERU3016308, UTERU3016789, UTERU3017176, UTERU3017441, UTERU3017626, UTERU3018154, UTERU3018255, UTERU3018616, UTERU3018711, UTERU3019078

Of the 17,176 genes that include the cDNAs of the present invention, genes whose expression levels were high enough to detect in each of the CD34C, CERVX, CHONS, COLON, CORDB, and CTONG libraries were analyzed for expression frequency. The analysis results are described below. Each is a relative value, where the expression level over all tissues is taken as 100. The larger this value, the greater the level of expression.

TABLE 29

| Clone Name | CD34C | CERVX | CHONS | COLON | CORDB | CTONG |
|---|---|---|---|---|---|---|
| 3NB691000116 | 0 | 0 | 0 | 0 | 0 | 6.058 |
| 3NB691000173 | 0 | 0 | 17.493 | 0 | 0 | 1.471 |
| 3NB691000191 | 0 | 0 | 0 | 42.978 | 0 | 0 |
| 3NB692000281 | 0 | 0 | 0 | 1.545 | 0 | 0 |
| 3NB692000912 | 0 | 0 | 0 | 3.299 | 0 | 0 |
| 3NB692001334 | 0 | 0 | 0 | 0 | 0 | 0.500 |
| 3NB692001339 | 0 | 0 | 0 | 16.465 | 0 | 0 |
| 3NB692001433 | 0 | 71.092 | 0 | 0 | 0 | 0 |
| ADRGL2000006 | 0 | 0 | 0 | 7.298 | 0 | 1.936 |
| ADRGL2001830 | 0 | 0 | 0 | 0 | 0 | 22.262 |
| ADRGL2002477 | 0 | 0 | 0 | 0 | 0 | 3.169 |
| ASTRO2000014 | 0 | 0 | 0 | 0 | 0 | 1.530 |
| ASTRO2000046 | 0 | 0 | 0 | 0 | 0 | 5.320 |
| ASTRO2001029 | 0 | 0 | 0 | 0 | 0 | 2.336 |
| ASTRO2001076 | 0 | 0 | 0 | 9.979 | 0 | 2.647 |
| ASTRO2001823 | 0 | 0 | 32.157 | 0 | 0 | 0 |
| ASTRO2003024 | 0 | 19.587 | 0 | 6.654 | 0 | 1.765 |
| ASTRO2003461 | 0 | 0 | 0 | 0 | 0 | 11.095 |
| ASTRO2003632 | 0 | 0 | 0 | 2.917 | 0 | 0.774 |
| ASTRO2004032 | 0 | 0 | 0 | 0 | 0 | 11.351 |
| ASTRO2005242 | 0 | 0 | 0 | 0 | 0 | 51.585 |
| ASTRO2005863 | 0 | 0 | 0 | 0 | 0 | 14.161 |
| ASTRO2006732 | 0 | 0 | 0 | 0 | 0 | 28.567 |
| ASTRO2007221 | 0 | 0 | 0 | 0 | 0 | 34.963 |
| ASTRO2007948 | 0 | 0 | 0 | 0 | 0 | 4.665 |
| ASTRO2008040 | 0 | 0 | 0 | 0 | 0 | 22.313 |
| ASTRO2008216 | 0 | 0 | 0 | 0 | 0 | 5.943 |
| ASTRO2010819 | 0 | 0 | 0 | 0 | 0 | 5.013 |
| ASTRO2015529 | 0 | 0 | 0 | 0 | 0 | 3.451 |
| ASTRO2015987 | 0 | 0 | 58.183 | 0 | 0 | 0 |
| ASTRO2016491 | 0 | 0 | 0 | 0 | 0 | 6.566 |
| BLADE2007666 | 0 | 70.405 | 0 | 0 | 0 | 0 |
| BNGH41000190 | 0 | 0 | 0 | 0 | 0 | 18.852 |
| BNGH41000198 | 0 | 0 | 0 | 0 | 0 | 3.870 |
| BNGH42000360 | 0 | 0 | 0 | 8.834 | 0 | 0 |
| BNGH42000532 | 0 | 0 | 0 | 0 | 0 | 17.614 |
| BNGH42002168 | 0 | 0 | 0 | 0 | 0 | 4.956 |
| BNGH42006135 | 0 | 0 | 0 | 0 | 0 | 1.230 |
| BNGH42007460 | 0 | 0 | 0 | 0 | 0 | 5.630 |
| BNGH42007709 | 0 | 4.757 | 0 | 0 | 0 | 0.429 |
| BNGH42007788 | 0 | 0 | 0 | 0 | 0 | 5.734 |
| BNGH42008743 | 0 | 0 | 0 | 0 | 0 | 2.867 |
| BRACE1000131 | 0 | 0 | 0 | 0 | 0 | 5.820 |
| BRACE2000307 | 0 | 0 | 0 | 0 | 0 | 2.194 |
| BRACE2000864 | 0 | 0 | 0 | 0 | 44.299 | 0.983 |
| BRACE2001117 | 0 | 0 | 0 | 19.309 | 0 | 0 |
| BRACE2001477 | 0 | 0 | 0 | 0 | 0 | 6.406 |
| BRACE2001534 | 0 | 63.743 | 0 | 21.655 | 0 | 0 |
| BRACE2001709 | 0 | 0 | 0 | 0 | 0 | 6.045 |
| BRACE2002091 | 0 | 0 | 0 | 0 | 0 | 3.933 |
| BRACE2002441 | 0 | 0 | 0 | 0 | 0 | 2.098 |
| BRACE2002649 | 0 | 0 | 0 | 0 | 0 | 19.720 |
| BRACE2002677 | 0 | 0 | 0 | 0 | 0 | 3.596 |
| BRACE2003078 | 0 | 0 | 0 | 0 | 0 | 30.148 |
| BRACE2003254 | 0 | 0 | 20.057 | 0 | 0 | 8.431 |
| BRACE2003304 | 0 | 0 | 0 | 0 | 0 | 12.216 |
| BRACE2003594 | 0 | 0 | 0 | 81.976 | 0 | 0 |
| BRACE2003785 | 0 | 40.163 | 0 | 0 | 0 | 0 |
| BRACE2003845 | 0 | 0 | 0 | 0 | 0 | 8.486 |
| BRACE2003846 | 0 | 0 | 0 | 0 | 0 | 1.214 |
| BRACE2003892 | 0 | 0 | 0 | 0 | 0 | 20.325 |
| BRACE2003904 | 0 | 0 | 0 | 0 | 0 | 4.829 |
| BRACE2005087 | 0 | 0 | 0 | 0 | 0 | 11.592 |
| BRACE2005448 | 0 | 0 | 0 | 47.739 | 0 | 0 |
| BRACE2005453 | 0 | 0 | 0 | 90.818 | 0 | 0 |
| BRACE2005642 | 0 | 0 | 0 | 51.298 | 0 | 0 |
| BRACE2006072 | 0 | 0 | 0 | 0 | 0 | 36.712 |
| BRACE2006105 | 0 | 0 | 0 | 0 | 0 | 39.515 |
| BRACE2006162 | 0 | 0 | 0 | 0 | 0 | 2.882 |
| BRACE2006319 | 0 | 0 | 0 | 0 | 0 | 6.530 |
| BRACE2006397 | 12.397 | 0 | 0 | 10.361 | 0 | 0.550 |
| BRACE2006514 | 0 | 0 | 0 | 0 | 0 | 1.618 |
| BRACE2006900 | 0 | 0 | 0 | 7.225 | 0 | 0 |
| BRACE2006937 | 0 | 27.657 | 0 | 0 | 0 | 0 |
| BRACE2007401 | 0 | 0 | 0 | 0 | 0 | 3.404 |
| BRACE2007518 | 0 | 0 | 0 | 15.934 | 0 | 0 |
| BRACE2007577 | 0 | 0 | 0 | 0 | 0 | 1.824 |
| BRACE2007671 | 0 | 0 | 0 | 0 | 0 | 2.880 |
| BRACE2008481 | 0 | 0 | 61.843 | 0 | 0 | 0 |
| BRACE2008653 | 0 | 0 | 0 | 0 | 0 | 6.876 |
| BRACE2009037 | 0 | 0 | 0 | 0 | 24.511 | 1.088 |
| BRACE2009366 | 0 | 74.665 | 0 | 0 | 0 | 0 |
| BRACE2009517 | 0 | 0 | 0 | 0 | 0 | 10.103 |
| BRACE2012528 | 0 | 0 | 0 | 0 | 0 | 0.883 |
| BRACE2016362 | 0 | 0 | 0 | 0 | 0 | 28.712 |
| BRACE2017397 | 0 | 48.741 | 0 | 0 | 0 | 0 |
| BRACE2021014 | 0 | 0 | 0 | 0 | 0 | 11.034 |
| BRACE2022638 | 0 | 0 | 0 | 0 | 0 | 1.388 |
| BRACE2027643 | 0 | 0 | 0 | 0 | 0 | 26.828 |
| BRACE2032477 | 0 | 0 | 0 | 0 | 0 | 10.379 |
| BRACE3001066 | 0 | 0 | 0 | 0 | 0 | 72.408 |
| BRACE3010428 | 0 | 0 | 0 | 0 | 0 | 6.396 |
| BRACE3013936 | 0 | 0 | 0 | 0 | 0 | 56.749 |
| BRALZ2009482 | 0 | 0 | 0 | 0 | 0 | 1.384 |
| BRAMY2000585 | 0 | 0 | 0 | 17.243 | 0 | 0 |
| BRAMY2002369 | 0 | 0 | 0 | 0 | 0 | 15.195 |
| BRAMY2004363 | 0 | 0 | 0 | 0 | 0 | 3.808 |
| BRAMY2004521 | 0 | 0 | 0 | 0 | 0 | 4.415 |
| BRAMY2007078 | 0 | 0 | 0 | 0 | 0 | 7.083 |
| BRAMY2007613 | 0 | 0 | 0 | 0 | 0 | 2.495 |
| BRAMY2009934 | 0 | 0 | 0 | 41.502 | 0 | 0 |
| BRAMY2012091 | 0 | 0 | 0 | 41.689 | 0 | 0 |
| BRAMY2015211 | 0 | 0 | 0 | 0 | 0 | 24.194 |
| BRAMY2019554 | 0 | 0 | 0 | 0 | 0 | 2.973 |
| BRAMY2020058 | 0 | 0 | 0 | 0 | 0 | 52.028 |
| BRAMY2020427 | 0 | 0 | 0 | 0 | 0 | 2.206 |
| BRAMY2022493 | 0 | 0 | 58.772 | 0 | 0 | 4.941 |
| BRAMY2026685 | 0 | 0 | 0 | 0 | 0 | 4.616 |
| BRAMY2026778 | 0 | 0 | 0 | 0 | 0 | 18.048 |
| BRAMY2027140 | 0 | 0 | 0 | 0 | 0 | 48.032 |
| BRAMY2027739 | 0 | 27.810 | 0 | 0 | 0 | 0 |
| BRAMY2028740 | 0 | 0 | 0 | 0 | 0 | 3.468 |
| BRAMY2032087 | 0 | 0 | 0 | 0 | 0 | 2.256 |
| BRAMY2040095 | 0 | 0 | 0 | 4.800 | 0 | 0 |
| BRAMY2040592 | 0 | 0 | 0 | 24.447 | 0 | 0 |
| BRAMY2047420 | 42.545 | 0 | 0 | 0 | 0 | 0 |
| BRAMY3002803 | 0 | 0 | 0 | 0 | 0 | 26.873 |
| BRAMY3004377 | 0 | 0 | 0 | 0 | 0 | 1.545 |
| BRAMY3010008 | 0 | 0 | 0 | 0 | 0 | 1.084 |
| BRAMY3010492 | 0 | 0 | 0 | 0 | 0 | 10.727 |
| BRAMY4001863 | 0 | 0 | 0 | 0 | 0 | 55.803 |
| BRAWH1000093 | 0 | 0 | 0 | 0 | 0 | 3.436 |
| BRAWH2000460 | 0 | 0 | 0 | 4.285 | 0 | 1.137 |
| BRAWH2000633 | 0 | 0 | 0 | 9.307 | 0 | 0 |
| BRAWH2000839 | 0 | 0 | 0 | 0 | 0 | 1.530 |
| BRAWH2001092 | 0 | 0 | 0 | 1.234 | 0 | 0.982 |
| BRAWH2001171 | 0 | 0 | 13.682 | 0 | 0 | 0 |
| BRAWH2001321 | 0 | 0 | 0 | 0 | 0 | 1.599 |
| BRAWH2001671 | 0 | 0 | 0 | 0 | 0 | 7.082 |
| BRAWH2003135 | 0 | 0 | 0 | 0 | 0 | 65.066 |
| BRAWH2003240 | 0 | 0 | 0 | 55.096 | 0 | 0 |
| BRAWH2005068 | 0 | 0 | 0 | 2.824 | 0 | 0 |
| BRAWH2005661 | 0 | 0 | 0 | 0 | 0 | 8.933 |
| BRAWH2008577 | 0 | 73.471 | 0 | 0 | 0 | 0 |
| BRAWH2008956 | 0 | 0 | 0 | 0 | 0 | 29.001 |
| BRAWH2009227 | 0 | 0 | 0 | 0 | 0 | 29.437 |
| BRAWH2009304 | 0 | 0 | 0 | 0 | 0 | 7.305 |
| BRAWH2014188 | 0 | 0 | 0 | 0 | 0 | 5.334 |
| BRAWH2014645 | 0 | 0 | 0 | 0 | 0 | 1.969 |
| BRAWH2017685 | 0 | 0 | 0 | 0 | 0 | 0.945 |
| BRAWH3002574 | 0 | 0 | 0 | 0 | 0 | 12.314 |
| BRAWH3002853 | 0 | 0 | 0 | 0 | 0 | 6.888 |
| BRAWH3021545 | 0 | 0 | 0 | 0 | 0 | 6.795 |
| BRAWH3021574 | 0 | 0 | 0 | 0 | 0 | 17.507 |
| BRCAN2000923 | 0 | 0 | 0 | 0 | 97.340 | 0 |
| BRCAN2003944 | 0 | 0 | 6.239 | 0 | 0 | 0 |
| BRCAN2009432 | 0 | 0 | 0 | 0 | 0 | 4.905 |
| BRCAN2015371 | 0 | 0 | 0 | 0 | 0 | 20.417 |
| BRCAN2015719 | 0 | 0 | 0 | 0 | 0 | 5.624 |
| BRCAN2020710 | 0 | 0 | 0 | 0 | 0 | 20.242 |
| BRCAN2024572 | 0 | 0 | 0 | 0 | 0 | 10.174 |
| BRCAN2026197 | 0 | 0 | 0 | 0 | 0 | 2.908 |
| BRCAN2028036 | 0 | 0 | 0 | 0 | 0 | 4.497 |
| BRCOC1000040 | 0 | 0 | 0 | 0 | 0 | 5.493 |
| BRCOC2000487 | 0 | 0 | 0 | 0 | 0 | 0.540 |
| BRCOC2003125 | 0 | 0 | 0 | 39.895 | 0 | 0 |
| BRCOC2003187 | 0 | 0 | 0 | 11.042 | 0 | 0 |
| BRCOC2010123 | 46.010 | 0 | 0 | 0 | 0 | 0 |
| BRCOC2019903 | 0 | 0 | 0 | 0 | 0 | 29.854 |
| BRHIP2000534 | 0 | 0 | 0 | 0 | 0 | 5.100 |
| BRHIP2000553 | 0 | 0 | 0 | 0 | 0 | 4.786 |
| BRHIP2002339 | 0 | 0 | 0 | 0 | 0 | 14.617 |
| BRHIP2004814 | 0 | 0 | 0 | 0 | 0 | 64.609 |
| BRHIP2007305 | 0 | 0 | 0 | 0 | 0 | 10.542 |
| BRHIP2017945 | 0 | 0 | 71.402 | 0 | 0 | 0 |
| BRHIP2022326 | 0 | 0 | 0 | 0 | 0 | 12.676 |
| BRHIP2023888 | 0 | 0 | 0 | 0 | 0 | 2.741 |
| BRHIP2024742 | 0 | 0 | 0 | 0 | 0 | 4.646 |
| BRHIP2027077 | 0 | 41.741 | 0 | 0 | 0 | 3.762 |
| BRHIP3000017 | 0 | 0 | 0 | 0 | 0 | 8.213 |
| BRHIP3007223 | 0 | 0 | 0 | 0 | 0 | 3.755 |
| BRHIP3012997 | 0 | 0 | 0 | 0 | 0 | 1.726 |
| BRHIP3020046 | 0 | 0 | 0 | 0 | 0 | 3.773 |
| BRSSN2000498 | 0 | 0 | 0 | 0 | 0 | 2.038 |
| BRSSN2009518 | 0 | 0 | 0 | 0 | 0 | 6.416 |
| BRSSN2013696 | 0 | 0 | 0 | 0 | 0 | 6.652 |
| BRSSN2014610 | 0 | 0 | 0 | 0 | 0 | 6.650 |
| BRSTN1000083 | 0 | 0 | 0 | 0 | 0 | 4.757 |
| BRSTN2011961 | 0 | 5.985 | 0 | 0 | 0 | 0.539 |
| BRSTN2012069 | 2.383 | 5.668 | 0.629 | 1.726 | 0.794 | 0.018 |
| BRSTN2012174 | 0 | 4.180 | 0 | 2.840 | 0 | 0 |
| BRSTN2013502 | 0 | 0 | 0 | 0 | 0 | 4.527 |
| BRSTN2016470 | 0 | 0 | 0 | 6.147 | 0 | 0 |
| BRTHA2006975 | 0 | 46.982 | 0 | 0 | 0 | 0 |
| BRTHA2007998 | 0 | 0 | 0 | 0 | 0 | 1.606 |
| BRTHA2016577 | 0 | 48.760 | 0 | 0 | 0 | 0 |
| BRTHA2027229 | 0 | 0 | 0 | 0 | 0 | 3.410 |
| BRTHA2033155 | 0 | 0 | 0 | 0 | 0 | 7.921 |
| BRTHA3011194 | 0 | 0 | 0 | 0 | 0 | 6.583 |
| BRTHA3020314 | 0 | 0 | 0 | 0 | 0 | 18.965 |
| BRTHA3022641 | 0 | 0 | 0 | 0 | 0 | 32.949 |
| CD34C2000051 | 100 | 0 | 0 | 0 | 0 | 0 |
| CD34C2000152 | 100 | 0 | 0 | 0 | 0 | 0 |
| CD34C2000175 | 100 | 0 | 0 | 0 | 0 | 0 |
| CD34C2000443 | 100 | 0 | 0 | 0 | 0 | 0 |
| CD34C3000125 | 97.628 | 0 | 0 | 0 | 0 | 0 |
| CD34C3000252 | 34.472 | 0 | 0 | 0 | 0 | 4.586 |
| CD34C3000314 | 100 | 0 | 0 | 0 | 0 | 0 |
| CD34C3000424 | 96.167 | 0 | 0 | 0 | 0 | 0 |
| CD34C3000494 | 100 | 0 | 0 | 0 | 0 | 0 |
| CERVX1000042 | 0 | 100 | 0 | 0 | 0 | 0 |
| CERVX2000812 | 0 | 100 | 0 | 0 | 0 | 0 |
| CERVX2000968 | 0 | 100 | 0 | 0 | 0 | 0 |
| CERVX2002006 | 0 | 100 | 0 | 0 | 0 | 0 |
| CERVX2002013 | 0 | 100 | 0 | 0 | 0 | 0 |
| CERVX2002430 | 0 | 100 | 0 | 0 | 0 | 0 |
| CERVX2002770 | 0 | 48.712 | 0 | 0 | 0 | 0 |
| CHONS1000058 | 0 | 0 | 100 | 0 | 0 | 0 |
| CHONS2000172 | 0 | 0 | 82.765 | 0 | 0 | 0 |
| CHONS2000797 | 0 | 0 | 96.373 | 0 | 0 | 0 |
| CHONS2001287 | 0 | 0 | 100 | 0 | 0 | 0 |
| CHONS2001382 | 0 | 0 | 100 | 0 | 0 | 0 |
| CHONS2001797 | 0 | 0 | 100 | 0 | 0 | 0 |
| CHONS2001834 | 0 | 0 | 100 | 0 | 0 | 0 |
| CHONS2002419 | 0 | 0 | 100 | 0 | 0 | 0 |
| CHONS2002829 | 0 | 0 | 36.193 | 0 | 0 | 0 |
| COLON1000030 | 0 | 0 | 0 | 100 | 0 | 0 |
| COLON1000084 | 0 | 0 | 0 | 100 | 0 | 0 |
| COLON1000135 | 0 | 0 | 0 | 100 | 0 | 0 |
| COLON2000470 | 0 | 0 | 0 | 50.006 | 0 | 0 |
| COLON2000557 | 0 | 0 | 0 | 100 | 0 | 0 |
| COLON2000568 | 0 | 0 | 0 | 100 | 0 | 0 |
| COLON2001721 | 0 | 0 | 0 | 38.840 | 0 | 0 |
| COLON2001829 | 0 | 0 | 0 | 100 | 0 | 0 |
| COLON2001866 | 0 | 0 | 0 | 100 | 0 | 0 |
| COLON2002443 | 0 | 0 | 0 | 31.658 | 0 | 0 |
| COLON2002520 | 0 | 0 | 0 | 100 | 0 | 0 |
| COLON2003043 | 0 | 0 | 0 | 100 | 0 | 0 |
| COLON2003529 | 0 | 0 | 0 | 100 | 0 | 0 |
| COLON2004318 | 0 | 0 | 0 | 100 | 0 | 0 |
| COLON2004351 | 0 | 0 | 0 | 100 | 0 | 0 |
| COLON2004478 | 0 | 0 | 0 | 100 | 0 | 0 |
| COLON2004911 | 0 | 0 | 0 | 100 | 0 | 0 |
| COLON2005126 | 0 | 0 | 0 | 100 | 0 | 0 |
| COLON2005623 | 0 | 0 | 0 | 100 | 0 | 0 |
| COLON2005735 | 0 | 0 | 0 | 100 | 0 | 0 |
| COLON2005772 | 0 | 0 | 0 | 100 | 0 | 0 |
| COLON2006263 | 0 | 0 | 0 | 100 | 0 | 0 |
| COLON2006282 | 0 | 0 | 0 | 100 | 0 | 0 |
| COLON2006417 | 0 | 0 | 0 | 100 | 0 | 0 |
| COLON2007274 | 0 | 0 | 0 | 100 | 0 | 0 |
| COLON2007733 | 0 | 0 | 0 | 87.532 | 0 | 0 |
| COLON2008105 | 0 | 0 | 0 | 100 | 0 | 0 |
| COLON2009337 | 0 | 0 | 0 | 100 | 0 | 0 |
| COLON2009499 | 0 | 0 | 0 | 100 | 0 | 0 |
| CORDB1000140 | 0 | 0 | 0 | 0 | 100 | 0 |
| CORDB2000061 | 0 | 0 | 0 | 0 | 100 | 0 |
| CORDB2000127 | 0 | 0 | 0 | 0 | 100 | 0 |
| CORDB2000541 | 0 | 0 | 0 | 0 | 100 | 0 |
| CTONG1000009 | 0 | 0 | 0 | 0 | 0 | 72.374 |
| CTONG1000010 | 0 | 0 | 0 | 0 | 0 | 12.075 |
| CTONG1000020 | 0 | 0 | 0 | 0 | 0 | 100 |
| CTONG1000022 | 0 | 0 | 0 | 0 | 0 | 100 |
| CTONG1000040 | 0 | 0 | 0 | 0 | 0 | 100 |
| CTONG1000052 | 0 | 0 | 0 | 0 | 0 | 5.627 |
| CTONG1000056 | 0 | 0 | 0 | 0 | 0 | 100 |
| CTONG1000062 | 0 | 0 | 0 | 0 | 0 | 100 |
| CTONG1000086 | 0 | 0 | 0 | 0 | 0 | 100 |
| CTONG1000087 | 0 | 0 | 0 | 0 | 0 | 3.895 |
| CTONG1000088 | 0 | 0 | 0 | 0 | 0 | 5.442 |
| CTONG1000093 | 0 | 0 | 0 | 0 | 0 | 74.021 |
| CTONG1000094 | 0 | 0 | 0 | 0 | 0 | 0.725 |
| CTONG1000097 | 0 | 0 | 0 | 0 | 0 | 100 |
| CTONG1000113 | 0 | 0 | 0 | 79.033 | 0 | 20.967 |
| CTONG1000137 | 0 | 0 | 0 | 0 | 0 | 60.963 |
| CTONG1000165 | 0 | 0 | 0 | 0 | 0 | 100 |
| CTONG1000175 | 0 | 0 | 0 | 0 | 0 | 15.679 |
| CTONG1000180 | 0 | 0 | 0 | 0 | 0 | 44.728 |
| CTONG1000181 | 0 | 0 | 0 | 0 | 0 | 100 |
| CTONG1000218 | 0 | 0 | 0 | 0 | 0 | 1.794 |
| CTONG1000220 | 0 | 0 | 0 | 0 | 0 | 24.817 |
| CTONG1000241 | 0 | 0 | 0 | 0 | 0 | 11.853 |
| CTONG1000277 | 0 | 0 | 0 | 0 | 0 | 38.523 |
| CTONG1000283 | 0 | 0 | 0 | 0 | 0 | 100 |
| CTONG1000288 | 0 | 0 | 0 | 0 | 0 | 84.986 |
| CTONG1000302 | 0 | 0 | 0 | 0 | 0 | 100 |
| CTONG1000341 | 0 | 0 | 0 | 0 | 0 | 51.706 |
| CTONG1000467 | 0 | 0 | 0 | 0 | 0 | 16.004 |
| CTONG1000476 | 0 | 0 | 0 | 0 | 0 | 100 |
| CTONG1000488 | 0 | 0 | 0 | 0 | 0 | 100 |
| CTONG1000508 | 0 | 0 | 0 | 0 | 0 | 100 |
| CTONG1000540 | 0 | 0 | 0 | 0 | 0 | 100 |
| CTONG2000031 | 0 | 0 | 0 | 0 | 0 | 65.066 |
| CTONG2000034 | 0 | 0 | 0 | 0 | 0 | 33.574 |
| CTONG2000042 | 0 | 0 | 0 | 0 | 0 | 65.252 |
| CTONG2000063 | 0 | 0 | 0 | 0 | 0 | 35.886 |
| CTONG2000201 | 0 | 0 | 0 | 13.608 | 0 | 21.662 |
| CTONG2000218 | 0 | 0 | 0 | 0 | 0 | 100 |
| CTONG2000279 | 0 | 0 | 0 | 0 | 0 | 100 |
| CTONG2000303 | 0 | 0 | 0 | 0 | 0 | 100 |
| CTONG2000330 | 0 | 0 | 0 | 0 | 0 | 100 |
| CTONG2000395 | 0 | 0 | 0 | 0 | 0 | 100 |
| CTONG2000411 | 0 | 0 | 0 | 0 | 0 | 64.312 |
| CTONG2000452 | 0 | 0 | 0 | 0 | 0 | 5.425 |
| CTONG2000465 | 0 | 0 | 0 | 0 | 0 | 69.076 |
| CTONG2000469 | 0 | 0 | 0 | 0 | 0 | 5.439 |
| CTONG2000480 | 0 | 0 | 0 | 0 | 0 | 100 |
| CTONG2000508 | 0 | 0 | 0 | 0 | 0 | 25.005 |
| CTONG2000561 | 0 | 0 | 0 | 0 | 0 | 40.551 |
| CTONG2000589 | 0 | 0 | 0 | 0 | 0 | 20.768 |
| CTONG2000766 | 0 | 0 | 0 | 0 | 0 | 100 |
| CTONG2000771 | 0 | 0 | 0 | 0 | 0 | 100 |
| CTONG2000819 | 0 | 0 | 0 | 0 | 0 | 100 |
| CTONG2000827 | 0 | 0 | 0 | 0 | 0 | 62.680 |
| CTONG2000846 | 0 | 0 | 0 | 0 | 0 | 100 |
| CTONG2000855 | 0 | 0 | 0 | 0 | 0 | 100 |
| CTONG2000977 | 0 | 0 | 0 | 0 | 0 | 100 |
| CTONG2001139 | 0 | 0 | 0 | 0 | 0 | 50.204 |
| CTONG2001226 | 0 | 0 | 0 | 0 | 0 | 14.138 |
| CTONG2001320 | 0 | 0 | 0 | 0 | 0 | 9.819 |
| CTONG2001366 | 0 | 0 | 0 | 0 | 0 | 28.875 |
| CTONG2001413 | 0 | 0 | 0 | 0 | 0 | 100 |
| CTONG2001428 | 0 | 0 | 0 | 0 | 0 | 62.446 |
| CTONG2001513 | 0 | 0 | 0 | 0 | 0 | 51.138 |
| CTONG2001524 | 0 | 0 | 0 | 0 | 0 | 100 |
| CTONG2001533 | 0 | 0 | 0 | 0 | 0 | 64.609 |
| CTONG2001598 | 0 | 0 | 0 | 0 | 0 | 72.408 |
| CTONG2001681 | 0 | 0 | 0 | 0 | 0 | 100 |
| CTONG2001724 | 0 | 0 | 0 | 0 | 0 | 100 |
| CTONG2001748 | 0 | 0 | 0 | 0 | 0 | 15.573 |
| CTONG2001749 | 0 | 0 | 0 | 0 | 0 | 100 |
| CTONG2001800 | 0 | 0 | 0 | 0 | 0 | 52.667 |
| CTONG2001820 | 0 | 0 | 0 | 0 | 0 | 50.616 |
| CTONG2001858 | 0 | 0 | 0 | 0 | 0 | 41.238 |
| CTONG2001877 | 0 | 0 | 0 | 0 | 0 | 100 |
| CTONG2001911 | 0 | 0 | 0 | 0 | 0 | 28.144 |
| CTONG2001932 | 0 | 0 | 0 | 0 | 0 | 38.485 |
| CTONG2001955 | 0 | 0 | 0 | 0 | 0 | 100 |
| CTONG2002066 | 0 | 0 | 0 | 79.033 | 0 | 20.967 |
| CTONG2002073 | 0 | 0 | 0 | 0 | 0 | 2.728 |
| CTONG2002095 | 0 | 0 | 0 | 0 | 0 | 26.088 |
| CTONG2002143 | 0 | 0 | 0 | 0 | 0 | 74.021 |
| CTONG2002270 | 0 | 0 | 0 | 0 | 0 | 12.843 |
| CTONG2002395 | 0 | 0 | 0 | 0 | 0 | 69.076 |
| CTONG2002417 | 0 | 0 | 0 | 0 | 0 | 100 |
| CTONG2002418 | 0 | 0 | 0 | 0 | 0 | 100 |
| CTONG2002453 | 0 | 0 | 0 | 0 | 0 | 14.460 |
| CTONG2002520 | 0 | 60.835 | 0 | 0 | 0 | 5.483 |
| CTONG2002558 | 0 | 0 | 0 | 0 | 0 | 51.706 |
| CTONG2002590 | 0 | 0 | 0 | 0 | 0 | 100 |
| CTONG2002709 | 0 | 0 | 0 | 0 | 0 | 4.036 |
| CTONG2002721 | 0 | 0 | 0 | 0 | 0 | 69.076 |
| CTONG2002744 | 0 | 0 | 13.047 | 0 | 0 | 9.872 |
| CTONG2002766 | 0 | 0 | 0 | 0 | 0 | 22.007 |
| CTONG2002803 | 0 | 0 | 0 | 0 | 0 | 2.742 |
| CTONG2002816 | 0 | 0 | 0 | 0 | 0 | 100 |
| CTONG2002820 | 0 | 0 | 0 | 0 | 0 | 41.898 |
| CTONG2002832 | 0 | 0 | 0 | 0 | 0 | 20.880 |
| CTONG2002841 | 0 | 0 | 0 | 0 | 0 | 19.729 |
| CTONG2002903 | 0 | 0 | 0 | 0 | 0 | 34.423 |
| CTONG2002965 | 0 | 0 | 0 | 0 | 0 | 100 |
| CTONG2002999 | 0 | 0 | 0 | 0 | 0 | 100 |
| CTONG2003028 | 0 | 0 | 0 | 0 | 0 | 100 |
| CTONG2003094 | 0 | 0 | 0 | 0 | 0 | 3.413 |
| CTONG2003100 | 0 | 0 | 0 | 0 | 0 | 7.981 |
| CTONG2003115 | 0 | 0 | 0 | 0 | 0 | 100 |
| CTONG2003189 | 0 | 0 | 0 | 0 | 0 | 74.021 |
| CTONG2003245 | 0 | 0 | 0 | 0 | 0 | 100 |
| CTONG2003293 | 0 | 0 | 0 | 0 | 0 | 17.217 |
| CTONG2003298 | 0 | 0 | 0 | 0 | 0 | 8.110 |
| CTONG2003348 | 0 | 0 | 0 | 0 | 0 | 60.963 |
| CTONG2003350 | 0 | 0 | 0 | 0 | 0 | 100 |
| CTONG2003361 | 0 | 0 | 0 | 0 | 0 | 43.764 |
| CTONG2003372 | 0 | 0 | 0 | 0 | 0 | 100 |
| CTONG2003375 | 0 | 0 | 0 | 0 | 0 | 100 |
| CTONG2003427 | 0 | 0 | 0 | 0 | 0 | 100 |
| CTONG2003517 | 0 | 0 | 0 | 0 | 0 | 20.880 |
| CTONG2003524 | 0 | 0 | 0 | 0 | 0 | 3.001 |
| CTONG2003599 | 0 | 0 | 0 | 0 | 0 | 100 |
| CTONG2003680 | 0 | 0 | 0 | 0 | 0 | 19.285 |
| CTONG2003699 | 0 | 0 | 0 | 0 | 0 | 64.894 |
| CTONG2003764 | 0 | 0 | 0 | 0 | 0 | 24.325 |
| CTONG2003782 | 0 | 0 | 0 | 0 | 0 | 100 |
| CTONG2003889 | 0 | 0 | 0 | 0 | 0 | 100 |
| CTONG2003937 | 0 | 0 | 0 | 0 | 0 | 34.822 |
| CTONG2003945 | 0 | 0 | 0 | 0 | 0 | 19.903 |
| CTONG2004000 | 0 | 0 | 0 | 0 | 0 | 100 |
| CTONG2004062 | 0 | 0 | 0 | 0 | 0 | 100 |
| CTONG2004115 | 0 | 0 | 0 | 0 | 0 | 22.262 |
| CTONG2004126 | 0 | 0 | 0 | 0 | 0 | 100 |
| CTONG2004264 | 0 | 0 | 0 | 0 | 0 | 100 |
| CTONG2004305 | 0 | 0 | 0 | 0 | 0 | 65.066 |
| CTONG2004397 | 0 | 0 | 0 | 0 | 0 | 51.706 |
| CTONG2004423 | 0 | 0 | 0 | 0 | 0 | 21.403 |
| CTONG2004454 | 0 | 0 | 0 | 0 | 0 | 100 |
| CTONG2004487 | 0 | 0 | 0 | 0 | 0 | 10.571 |
| CTONG2004550 | 0 | 0 | 0 | 0 | 0 | 34.822 |
| CTONG2004669 | 0 | 0 | 0 | 0 | 0 | 100 |
| CTONG2004716 | 0 | 0 | 0 | 0 | 0 | 100 |
| CTONG2004912 | 0 | 0 | 0 | 0 | 0 | 100 |
| CTONG2004941 | 0 | 0 | 0 | 0 | 0 | 100 |
| CTONG2004948 | 0 | 0 | 0 | 0 | 0 | 100 |
| CTONG2005028 | 0 | 0 | 0 | 0 | 0 | 25.499 |
| CTONG2005049 | 0 | 0 | 0 | 0 | 0 | 100 |
| CTONG2005110 | 0 | 0 | 0 | 0 | 0 | 100 |
| CTONG2005145 | 0 | 0 | 0 | 0 | 0 | 45.702 |
| CTONG2005265 | 0 | 0 | 0 | 0 | 0 | 34.822 |
| CTONG2005278 | 0 | 0 | 0 | 0 | 0 | 10.736 |
| CTONG2005290 | 0 | 0 | 0 | 0 | 0 | 42.764 |
| CTONG2005374 | 0 | 0 | 0 | 0 | 0 | 100 |
| CTONG2005399 | 0 | 0 | 0 | 0 | 0 | 100 |
| CTONG2005468 | 0 | 0 | 0 | 0 | 0 | 83.301 |
| CTONG2005553 | 0 | 0 | 0 | 0 | 0 | 9.337 |
| CTONG2005567 | 0 | 0 | 0 | 0 | 0 | 100 |
| CTONG2005585 | 0 | 0 | 0 | 0 | 0 | 5.218 |
| CTONG2005615 | 0 | 0 | 0 | 0 | 0 | 17.713 |
| CTONG2005635 | 0 | 0 | 0 | 0 | 0 | 100 |
| CTONG2005775 | 0 | 0 | 0 | 0 | 0 | 100 |
| CTONG2005795 | 0 | 0 | 0 | 0 | 0 | 64.609 |
| CTONG2005890 | 0 | 0 | 0 | 0 | 0 | 100 |
| CTONG2005904 | 0 | 0 | 0 | 0 | 0 | 100 |
| CTONG2005913 | 0 | 0 | 0 | 0 | 0 | 49.868 |
| CTONG2006004 | 0 | 0 | 0 | 0 | 0 | 3.932 |
| CTONG2006129 | 0 | 0 | 0 | 0 | 0 | 100 |
| CTONG2006223 | 0 | 0 | 0 | 0 | 0 | 100 |
| CTONG2006235 | 0 | 0 | 0 | 0 | 0 | 3.051 |
| CTONG2006273 | 0 | 0 | 0 | 0 | 0 | 100 |
| CTONG2006310 | 0 | 0 | 0 | 0 | 0 | 100 |
| CTONG2006377 | 0 | 0 | 0 | 0 | 0 | 21.867 |
| CTONG2006393 | 0 | 0 | 0 | 0 | 0 | 65.066 |
| CTONG2006412 | 0 | 0 | 0 | 0 | 0 | 11.562 |
| CTONG2006449 | 0 | 0 | 0 | 0 | 0 | 16.373 |
| CTONG2006515 | 0 | 0 | 0 | 0 | 0 | 100 |
| CTONG2006524 | 0 | 0 | 0 | 0 | 0 | 100 |
| CTONG2006562 | 0 | 0 | 0 | 0 | 0 | 7.823 |
| CTONG2006568 | 0 | 0 | 0 | 0 | 0 | 100 |
| CTONG2006611 | 0 | 0 | 0 | 0 | 0 | 26.900 |
| CTONG2006620 | 0 | 0 | 0 | 0 | 0 | 100 |
| CTONG2006666 | 0 | 0 | 0 | 0 | 0 | 48.802 |
| CTONG2006691 | 0 | 0 | 0 | 0 | 0 | 100 |
| CTONG2006709 | 0 | 0 | 0 | 0 | 0 | 9.633 |
| CTONG2006798 | 0 | 0 | 0 | 0 | 0 | 17.972 |
| CTONG2006836 | 0 | 0 | 0 | 0 | 0 | 100 |
| CTONG2006932 | 0 | 0 | 24.378 | 0 | 0 | 4.099 |
| CTONG2006942 | 0 | 0 | 0 | 0 | 0 | 100 |
| CTONG2006964 | 0 | 0 | 0 | 0 | 0 | 100 |
| CTONG2007007 | 0 | 0 | 0 | 0 | 0 | 69.356 |
| CTONG2007009 | 0 | 0 | 0 | 0 | 0 | 100 |
| CTONG2007072 | 0 | 0 | 0 | 0 | 0 | 12.021 |
| CTONG2007078 | 0 | 0 | 0 | 0 | 0 | 2.380 |
| CTONG2007091 | 0 | 0 | 0 | 0 | 0 | 100 |
| CTONG2007104 | 0 | 0 | 0 | 0 | 0 | 23.169 |
| CTONG2007168 | 0 | 0 | 0 | 0 | 0 | 11.259 |
| CTONG2007175 | 0 | 0 | 0 | 0 | 0 | 100 |
| CTONG2007259 | 0 | 0 | 0 | 0 | 0 | 100 |
| CTONG2007272 | 0 | 0 | 0 | 0 | 0 | 100 |
| CTONG2007293 | 0 | 0 | 0 | 0 | 0 | 62.680 |
| CTONG2007297 | 0 | 0 | 0 | 0 | 0 | 100 |
| CTONG2007399 | 0 | 0 | 0 | 0 | 0 | 30.379 |
| CTONG2007400 | 0 | 0 | 0 | 0 | 0 | 42.865 |
| CTONG2007417 | 0 | 0 | 0 | 0 | 0 | 40.148 |
| CTONG2007441 | 0 | 0 | 0 | 0 | 0 | 76.882 |
| CTONG2007474 | 0 | 0 | 0 | 0 | 0 | 19.785 |
| CTONG2007500 | 0 | 0 | 0 | 5.280 | 0 | 2.801 |
| CTONG2007586 | 0 | 0 | 0 | 0 | 0 | 11.194 |
| CTONG2007613 | 0 | 0 | 0 | 0 | 0 | 16.303 |
| CTONG2007623 | 0 | 0 | 0 | 0 | 0 | 100 |
| CTONG2007681 | 0 | 0 | 0 | 0 | 0 | 34.420 |
| CTONG2007776 | 0 | 0 | 0 | 0 | 0 | 7.922 |
| CTONG2007779 | 0 | 0 | 0 | 0 | 0 | 62.680 |
| CTONG2007898 | 0 | 0 | 0 | 0 | 0 | 100 |
| CTONG2007959 | 0 | 0 | 0 | 0 | 0 | 33.883 |
| CTONG2007970 | 0 | 0 | 0 | 0 | 0 | 100 |
| CTONG2008014 | 0 | 0 | 0 | 0 | 0 | 28.200 |
| CTONG2008184 | 0 | 0 | 0 | 0 | 0 | 100 |
| CTONG2008262 | 0 | 0 | 0 | 0 | 0 | 100 |
| CTONG2008269 | 0 | 0 | 0 | 0 | 0 | 12.672 |
| CTONG2008321 | 0 | 0 | 0 | 0 | 0 | 28.019 |
| CTONG2008343 | 0 | 0 | 0 | 0 | 0 | 60.963 |
| CTONG2008398 | 32.111 | 0 | 0 | 0 | 0 | 1.424 |
| CTONG2008402 | 0 | 0 | 0 | 0 | 0 | 100 |
| CTONG2008405 | 0 | 0 | 0 | 0 | 0 | 40.989 |
| CTONG2008466 | 0 | 0 | 0 | 0 | 0 | 14.562 |
| CTONG2008506 | 0 | 0 | 0 | 0 | 0 | 3.505 |
| CTONG2008518 | 0 | 0 | 0 | 0 | 0 | 21.520 |
| CTONG2008521 | 0 | 0 | 0 | 0 | 0 | 28.316 |
| CTONG2008562 | 0 | 0 | 0 | 0 | 0 | 100 |
| CTONG2008577 | 0 | 0 | 0 | 0 | 0 | 100 |
| CTONG2008595 | 0 | 0 | 0 | 0 | 0 | 100 |
| CTONG2008621 | 0 | 0 | 0 | 0 | 0 | 62.446 |
| CTONG2008689 | 0 | 0 | 0 | 0 | 0 | 38.923 |
| CTONG2008720 | 0 | 0 | 0 | 0 | 0 | 50.285 |
| CTONG2008721 | 0 | 0 | 0 | 0 | 0 | 62.680 |
| CTONG2008773 | 0 | 0 | 0 | 0 | 0 | 100 |
| CTONG2008792 | 0 | 0 | 0 | 0 | 0 | 13.686 |
| CTONG2008989 | 0 | 0 | 0 | 0 | 0 | 100 |
| CTONG2009033 | 0 | 0 | 0 | 0 | 0 | 47.970 |
| CTONG2009108 | 0 | 0 | 0 | 0 | 0 | 44.201 |
| CTONG2009132 | 0 | 0 | 0 | 0 | 0 | 100 |
| CTONG2009156 | 0 | 0 | 0 | 0 | 0 | 100 |
| CTONG2009181 | 0 | 0 | 0 | 0 | 0 | 100 |
| CTONG2009257 | 0 | 0 | 0 | 0 | 0 | 100 |
| CTONG2009258 | 0 | 0 | 0 | 0 | 0 | 100 |
| CTONG2009268 | 0 | 0 | 0 | 0 | 0 | 100 |
| CTONG2009270 | 0 | 0 | 0 | 0 | 0 | 100 |
| CTONG2009395 | 0 | 0 | 0 | 0 | 0 | 100 |
| CTONG2009423 | 0 | 0 | 0 | 0 | 0 | 64.609 |
| CTONG2009440 | 0 | 0 | 0 | 0 | 0 | 100 |
| CTONG2009527 | 0 | 0 | 0 | 0 | 0 | 100 |
| CTONG2009529 | 0 | 0 | 0 | 0 | 0 | 11.777 |
| CTONG2009531 | 0 | 0 | 0 | 0 | 0 | 100 |
| CTONG2009534 | 0 | 0 | 0 | 0 | 0 | 14.323 |
| CTONG2009570 | 0 | 0 | 0 | 0 | 0 | 100 |
| CTONG2009572 | 0 | 0 | 0 | 0 | 0 | 100 |
| CTONG2009604 | 0 | 0 | 0 | 0 | 0 | 100 |
| CTONG2009643 | 0 | 0 | 0 | 0 | 0 | 100 |
| CTONG2009675 | 0 | 0 | 0 | 0 | 0 | 100 |
| CTONG2009689 | 0 | 0 | 0 | 0 | 0 | 100 |
| CTONG2009725 | 0 | 0 | 0 | 0 | 0 | 100 |
| CTONG2009766 | 0 | 0 | 0 | 0 | 0 | 100 |
| CTONG2009768 | 0 | 0 | 0 | 0 | 0 | 100 |
| CTONG2009844 | 0 | 0 | 0 | 0 | 0 | 33.474 |
| CTONG2009871 | 0 | 0 | 0 | 0 | 0 | 100 |
| CTONG2009887 | 0 | 0 | 0 | 0 | 0 | 100 |
| CTONG2009891 | 0 | 0 | 0 | 0 | 0 | 100 |
| CTONG2009923 | 0 | 0 | 0 | 0 | 0 | 60.963 |
| CTONG2009938 | 0 | 0 | 0 | 0 | 0 | 37.266 |
| CTONG2009955 | 0 | 0 | 0 | 0 | 0 | 100 |
| CTONG2009958 | 0 | 0 | 0 | 0 | 0 | 100 |
| CTONG2009963 | 0 | 0 | 0 | 0 | 0 | 38.502 |
| CTONG2010018 | 0 | 0 | 0 | 0 | 0 | 100 |
| CTONG2010024 | 0 | 0 | 0 | 0 | 0 | 100 |
| CTONG2010116 | 0 | 0 | 0 | 0 | 0 | 100 |
| CTONG2010133 | 0 | 0 | 0 | 0 | 0 | 100 |
| CTONG2010148 | 0 | 0 | 0 | 0 | 0 | 100 |
| CTONG2010276 | 0 | 0 | 0 | 0 | 0 | 100 |
| CTONG2010330 | 0 | 0 | 0 | 0 | 0 | 100 |
| CTONG2010348 | 0 | 0 | 0 | 0 | 0 | 13.193 |
| CTONG2010408 | 0 | 0 | 0 | 0 | 0 | 15.959 |
| CTONG2010508 | 0 | 84.727 | 0 | 0 | 0 | 15.273 |
| CTONG2010566 | 0 | 0 | 0 | 0 | 0 | 25.256 |
| CTONG2010623 | 0 | 0 | 0 | 0 | 0 | 100 |
| CTONG2010649 | 0 | 32.315 | 0 | 0 | 0 | 8.737 |
| CTONG2010652 | 0 | 0 | 0 | 0 | 0 | 29.937 |
| CTONG2010803 | 0 | 0 | 0 | 0 | 0 | 20.971 |
| CTONG2010821 | 0 | 0 | 0 | 0 | 0 | 2.662 |
| CTONG2011188 | 0 | 0 | 0 | 0 | 0 | 78.500 |
| CTONG2011429 | 0 | 0 | 0 | 0 | 0 | 100 |
| CTONG2011474 | 0 | 0 | 0 | 0 | 0 | 100 |
| CTONG2011676 | 0 | 0 | 0 | 0 | 0 | 100 |
| CTONG2011716 | 0 | 0 | 0 | 0 | 0 | 51.706 |
| CTONG2011765 | 0 | 0 | 0 | 0 | 0 | 100 |
| CTONG2011779 | 0 | 0 | 0 | 0 | 0 | 100 |
| CTONG2011801 | 0 | 0 | 0 | 0 | 0 | 5.515 |
| CTONG2011815 | 0 | 0 | 0 | 0 | 0 | 100 |
| CTONG2011822 | 0 | 0 | 0 | 0 | 0 | 100 |
| CTONG2011825 | 0 | 0 | 0 | 0 | 0 | 15.991 |
| CTONG2011920 | 0 | 0 | 0 | 0 | 0 | 100 |
| CTONG2011985 | 0 | 0 | 0 | 0 | 0 | 100 |
| CTONG2012001 | 0 | 0 | 0 | 0 | 0 | 13.318 |
| CTONG2012029 | 0 | 0 | 0 | 0 | 0 | 34.822 |
| CTONG2012050 | 0 | 0 | 0 | 0 | 0 | 13.231 |
| CTONG2012077 | 0 | 0 | 0 | 0 | 0 | 100 |
| CTONG2012101 | 0 | 0 | 0 | 0 | 0 | 29.404 |
| CTONG2012123 | 0 | 0 | 0 | 0 | 0 | 100 |
| CTONG2012158 | 0 | 0 | 0 | 0 | 0 | 33.983 |
| CTONG2012230 | 0 | 0 | 0 | 0 | 0 | 100 |
| CTONG2012401 | 0 | 0 | 0 | 0 | 0 | 7.835 |
| CTONG2012422 | 0 | 0 | 0 | 0 | 0 | 69.076 |
| CTONG2012425 | 0 | 0 | 0 | 0 | 0 | 51.706 |
| CTONG2012447 | 0 | 0 | 0 | 0 | 0 | 100 |
| CTONG2012452 | 0 | 0 | 0 | 0 | 0 | 1.326 |
| CTONG2012453 | 0 | 0 | 0 | 0 | 0 | 14.027 |
| CTONG2012473 | 0 | 0 | 0 | 0 | 0 | 100 |
| CTONG2012476 | 0 | 0 | 0 | 0 | 0 | 100 |
| CTONG2012533 | 0 | 0 | 0 | 0 | 0 | 100 |
| CTONG2012554 | 0 | 0 | 0 | 0 | 0 | 100 |
| CTONG2012564 | 0 | 0 | 0 | 0 | 0 | 7.359 |
| CTONG2012607 | 0 | 0 | 0 | 0 | 0 | 3.221 |
| CTONG2012661 | 0 | 0 | 0 | 0 | 0 | 39.027 |
| CTONG2012724 | 0 | 0 | 0 | 0 | 0 | 100 |
| CTONG2012738 | 0 | 0 | 0 | 0 | 0 | 100 |
| CTONG2012745 | 0 | 0 | 0 | 0 | 0 | 10.331 |
| CTONG2012758 | 0 | 0 | 0 | 0 | 0 | 100 |
| CTONG2012843 | 0 | 0 | 0 | 0 | 0 | 33.574 |
| CTONG2012847 | 0 | 0 | 0 | 0 | 0 | 9.622 |
| CTONG2012879 | 0 | 0 | 0 | 0 | 0 | 19.002 |
| CTONG2012901 | 0 | 0 | 0 | 0 | 0 | 100 |
| CTONG2012996 | 0 | 0 | 0 | 0 | 0 | 32.630 |
| CTONG2013128 | 0 | 0 | 0 | 0 | 0 | 60.963 |
| CTONG2013149 | 0 | 0 | 0 | 0 | 0 | 24.817 |
| CTONG2013156 | 0 | 0 | 0 | 0 | 0 | 38.523 |
| CTONG2013178 | 0 | 0 | 0 | 0 | 0 | 35.252 |
| CTONG2013222 | 0 | 0 | 0 | 0 | 0 | 6.999 |
| CTONG2013284 | 0 | 0 | 0 | 0 | 0 | 100 |
| CTONG2013339 | 0 | 0 | 0 | 0 | 0 | 100 |
| CTONG2013348 | 0 | 0 | 0 | 0 | 0 | 10.246 |
| CTONG2013352 | 0 | 0 | 0 | 0 | 0 | 50.616 |
| CTONG2013381 | 0 | 0 | 0 | 0 | 0 | 29.215 |
| CTONG2013393 | 0 | 0 | 0 | 0 | 0 | 100 |
| CTONG2013511 | 0 | 0 | 0 | 0 | 0 | 100 |
| CTONG2013630 | 0 | 0 | 0 | 0 | 0 | 100 |
| CTONG2013803 | 0 | 0 | 0 | 0 | 0 | 100 |
| CTONG2013907 | 0 | 0 | 0 | 0 | 0 | 8.031 |
| CTONG2013934 | 0 | 0 | 0 | 0 | 0 | 12.919 |
| CTONG2013985 | 0 | 0 | 0 | 0 | 0 | 21.815 |
| CTONG2013986 | 0 | 0 | 0 | 0 | 0 | 100 |
| CTONG2014032 | 0 | 0 | 0 | 0 | 0 | 100 |
| CTONG2014058 | 0 | 0 | 0 | 0 | 0 | 100 |
| CTONG2014119 | 0 | 0 | 0 | 0 | 0 | 30.190 |
| CTONG2014165 | 0 | 0 | 0 | 0 | 0 | 8.968 |
| CTONG2014191 | 0 | 0 | 0 | 0 | 0 | 100 |
| CTONG2014206 | 0 | 0 | 0 | 0 | 0 | 100 |
| CTONG2014234 | 0 | 0 | 0 | 0 | 0 | 100 |
| CTONG2014264 | 0 | 0 | 0 | 0 | 0 | 100 |
| CTONG2014280 | 0 | 0 | 0 | 0 | 0 | 23.412 |
| CTONG2014369 | 0 | 0 | 0 | 0 | 0 | 23.253 |
| CTONG2014389 | 0 | 0 | 0 | 0 | 0 | 4.920 |
| CTONG2014491 | 0 | 0 | 0 | 0 | 0 | 62.446 |
| CTONG2014630 | 0 | 0 | 0 | 0 | 0 | 100 |
| CTONG2014635 | 0 | 0 | 0 | 0 | 0 | 100 |
| CTONG2014646 | 0 | 0 | 0 | 0 | 0 | 100 |
| CTONG2014705 | 0 | 0 | 0 | 0 | 0 | 34.963 |
| CTONG2014898 | 0 | 0 | 0 | 0 | 0 | 10.743 |
| CTONG2014946 | 0 | 0 | 0 | 0 | 0 | 100 |
| CTONG2014954 | 0 | 0 | 0 | 0 | 0 | 22.772 |
| CTONG2014959 | 0 | 0 | 0 | 0 | 0 | 100 |
| CTONG2014966 | 0 | 0 | 0 | 0 | 0 | 100 |
| CTONG2014995 | 0 | 0 | 0 | 0 | 0 | 100 |
| CTONG2015091 | 0 | 0 | 0 | 0 | 0 | 3.025 |
| CTONG2015204 | 0 | 0 | 0 | 0 | 0 | 56.503 |
| CTONG2015316 | 0 | 0 | 0 | 0 | 0 | 100 |
| CTONG2015330 | 0 | 0 | 0 | 0 | 0 | 52.970 |
| CTONG2015345 | 0 | 0 | 0 | 0 | 0 | 31.801 |
| CTONG2015358 | 0 | 0 | 0 | 0 | 0 | 74.021 |
| CTONG2015434 | 0 | 0 | 0 | 0 | 0 | 100 |
| CTONG2015518 | 0 | 0 | 0 | 0 | 0 | 24.734 |
| CTONG2015540 | 0 | 0 | 0 | 0 | 0 | 100 |
| CTONG2015596 | 0 | 0 | 0 | 0 | 0 | 100 |
| CTONG2015633 | 0 | 0 | 0 | 0 | 0 | 100 |
| CTONG2015678 | 0 | 0 | 0 | 0 | 0 | 62.446 |
| CTONG2015717 | 0 | 0 | 0 | 0 | 0 | 100 |
| CTONG2015804 | 0 | 0 | 0 | 0 | 0 | 100 |
| CTONG2015815 | 0 | 0 | 0 | 0 | 0 | 16.385 |
| CTONG2015866 | 0 | 0 | 0 | 0 | 0 | 100 |
| CTONG2015914 | 0 | 0 | 0 | 0 | 0 | 100 |
| CTONG2015953 | 0 | 0 | 0 | 0 | 0 | 100 |
| CTONG2016056 | 0 | 0 | 0 | 0 | 0 | 3.704 |
| CTONG2016185 | 0 | 0 | 0 | 0 | 0 | 19.368 |
| CTONG2016217 | 0 | 0 | 0 | 0 | 0 | 100 |
| CTONG2016355 | 0 | 0 | 0 | 0 | 0 | 100 |
| CTONG2016408 | 0 | 0 | 0 | 0 | 0 | 6.896 |
| CTONG2016499 | 0 | 0 | 0 | 0 | 0 | 65.066 |
| CTONG2016505 | 0 | 0 | 0 | 0 | 0 | 33.474 |
| CTONG2016559 | 0 | 0 | 0 | 0 | 0 | 100 |
| CTONG2016575 | 0 | 0 | 0 | 0 | 0 | 7.408 |
| CTONG2016658 | 0 | 0 | 0 | 0 | 0 | 100 |
| CTONG2016775 | 0 | 0 | 0 | 0 | 0 | 100 |
| CTONG2016797 | 0 | 0 | 0 | 0 | 0 | 100 |
| CTONG2016824 | 0 | 0 | 0 | 0 | 0 | 7.928 |
| CTONG2016846 | 0 | 0 | 0 | 0 | 0 | 100 |
| CTONG2016869 | 0 | 0 | 0 | 0 | 0 | 29.516 |
| CTONG2016904 | 0 | 0 | 0 | 0 | 0 | 100 |
| CTONG2016931 | 0 | 0 | 0 | 0 | 0 | 11.368 |
| CTONG2016942 | 0 | 0 | 0 | 0 | 0 | 21.398 |
| CTONG2016953 | 0 | 0 | 0 | 0 | 0 | 100 |
| CTONG2017021 | 0 | 0 | 0 | 0 | 0 | 100 |
| CTONG2017094 | 0 | 0 | 0 | 0 | 0 | 70.789 |
| CTONG2017292 | 0 | 0 | 0 | 0 | 0 | 100 |
| CTONG2017409 | 0 | 0 | 0 | 0 | 0 | 62.680 |
| CTONG2017429 | 0 | 0 | 0 | 0 | 0 | 40.325 |
| CTONG2017444 | 0 | 0 | 0 | 0 | 0 | 34.294 |
| CTONG2017458 | 0 | 0 | 0 | 0 | 0 | 100 |
| CTONG2017500 | 0 | 0 | 0 | 0 | 0 | 4.934 |
| CTONG2017604 | 0 | 0 | 0 | 0 | 0 | 22.375 |
| CTONG2017650 | 0 | 0 | 0 | 0 | 0 | 100 |
| CTONG2017798 | 0 | 0 | 0 | 0 | 0 | 37.119 |
| CTONG2017804 | 0 | 0 | 0 | 0 | 0 | 100 |
| CTONG2017814 | 0 | 0 | 0 | 0 | 0 | 100 |
| CTONG2017910 | 0 | 0 | 0 | 0 | 0 | 100 |
| CTONG2017939 | 0 | 0 | 0 | 0 | 0 | 100 |
| CTONG2017989 | 0 | 0 | 0 | 0 | 0 | 100 |
| CTONG2017998 | 0 | 0 | 0 | 0 | 0 | 4.666 |
| CTONG2018062 | 0 | 0 | 0 | 0 | 0 | 100 |
| CTONG2018069 | 0 | 0 | 0 | 0 | 0 | 100 |
| CTONG2018147 | 0 | 0 | 0 | 0 | 0 | 100 |
| CTONG2018211 | 0 | 0 | 0 | 0 | 0 | 6.352 |
| CTONG2018217 | 0 | 0 | 0 | 0 | 0 | 100 |
| CTONG2018279 | 0 | 0 | 0 | 0 | 0 | 100 |
| CTONG2018334 | 0 | 0 | 0 | 51.354 | 0 | 13.624 |
| CTONG2018343 | 0 | 0 | 0 | 0 | 0 | 28.371 |
| CTONG2018379 | 0 | 0 | 0 | 0 | 0 | 100 |
| CTONG2018383 | 0 | 0 | 0 | 0 | 0 | 16.839 |
| CTONG2018413 | 0 | 0 | 0 | 0 | 0 | 46.898 |
| CTONG2018483 | 0 | 0 | 0 | 0 | 0 | 100 |
| CTONG2018629 | 0 | 0 | 0 | 0 | 0 | 76.882 |
| CTONG2018632 | 0 | 0 | 0 | 0 | 0 | 14.897 |
| CTONG2018637 | 0 | 0 | 0 | 0 | 0 | 12.219 |
| CTONG2018652 | 0 | 0 | 0 | 0 | 0 | 74.021 |
| CTONG2018653 | 0 | 0 | 0 | 0 | 0 | 100 |
| CTONG2018655 | 0 | 0 | 0 | 0 | 0 | 100 |
| CTONG2018735 | 0 | 0 | 0 | 0 | 0 | 69.076 |
| CTONG2018739 | 0 | 0 | 0 | 0 | 0 | 7.218 |
| CTONG2018808 | 0 | 0 | 0 | 0 | 0 | 14.670 |
| CTONG2018843 | 0 | 0 | 0 | 0 | 0 | 20.678 |
| CTONG2018898 | 0 | 0 | 0 | 0 | 0 | 0.897 |
| CTONG2018900 | 0 | 0 | 0 | 0 | 0 | 7.970 |
| CTONG2018976 | 0 | 0 | 0 | 0 | 0 | 69.076 |
| CTONG2019029 | 0 | 0 | 0 | 0 | 0 | 60.288 |
| CTONG2019063 | 0 | 0 | 0 | 0 | 0 | 39.545 |
| CTONG2019144 | 0 | 0 | 0 | 0 | 0 | 100 |
| CTONG2019232 | 0 | 0 | 0 | 0 | 0 | 100 |
| CTONG2019248 | 0 | 0 | 0 | 0 | 0 | 28.179 |
| CTONG2019400 | 0 | 0 | 0 | 0 | 0 | 36.767 |
| CTONG2019590 | 0 | 0 | 0 | 0 | 0 | 20.042 |
| CTONG2019652 | 0 | 0 | 0 | 0 | 0 | 100 |
| CTONG2019704 | 0 | 0 | 0 | 0 | 0 | 62.446 |
| CTONG2019788 | 0 | 0 | 0 | 0 | 0 | 11.390 |
| CTONG2019822 | 0 | 0 | 0 | 0 | 0 | 12.111 |
| CTONG2019833 | 0 | 0 | 0 | 0 | 0 | 100 |
| CTONG2020031 | 0 | 0 | 0 | 0 | 0 | 100 |
| CTONG2020108 | 0 | 0 | 0 | 0 | 0 | 69.076 |
| CTONG2020127 | 0 | 0 | 0 | 0 | 0 | 100 |
| CTONG2020171 | 0 | 0 | 0 | 0 | 0 | 100 |
| CTONG2020374 | 0 | 0 | 0 | 0 | 0 | 57.889 |
| CTONG2020378 | 0 | 0 | 0 | 0 | 0 | 30.063 |
| CTONG2020411 | 0 | 0 | 0 | 0 | 0 | 12.823 |
| CTONG2020445 | 0 | 0 | 0 | 0 | 0 | 39.202 |
| CTONG2020484 | 0 | 0 | 0 | 0 | 0 | 1.095 |
| CTONG2020522 | 0 | 0 | 0 | 0 | 0 | 42.683 |
| CTONG2020560 | 0 | 0 | 0 | 0 | 0 | 2.794 |
| CTONG2020582 | 0 | 0 | 0 | 0 | 0 | 21.442 |
| CTONG2020638 | 0 | 0 | 0 | 0 | 0 | 23.060 |
| CTONG2020806 | 0 | 0 | 0 | 0 | 0 | 100 |
| CTONG2020831 | 0 | 0 | 0 | 0 | 0 | 62.680 |
| CTONG2020974 | 0 | 0 | 0 | 0 | 0 | 74.021 |
| CTONG2021024 | 0 | 0 | 0 | 0 | 0 | 62.446 |
| CTONG2021132 | 0 | 0 | 0 | 0 | 0 | 100 |
| CTONG2021168 | 0 | 0 | 0 | 0 | 0 | 100 |
| CTONG2021289 | 0 | 0 | 0 | 0 | 0 | 19.164 |
| CTONG2022153 | 0 | 0 | 0 | 0 | 0 | 100 |
| CTONG2022601 | 0 | 0 | 0 | 0 | 0 | 100 |
| CTONG2022835 | 72.054 | 0 | 0 | 0 | 0 | 3.195 |
| CTONG2023021 | 0 | 0 | 0 | 0 | 0 | 62.680 |
| CTONG2023449 | 0 | 0 | 0 | 0 | 0 | 21.398 |
| CTONG2023512 | 0 | 0 | 0 | 0 | 0 | 100 |
| CTONG2024031 | 0 | 0 | 0 | 0 | 0 | 19.255 |
| CTONG2024206 | 0 | 0 | 0 | 0 | 0 | 100 |
| CTONG2024614 | 0 | 0 | 0 | 0 | 0 | 100 |
| CTONG2024749 | 0 | 0 | 0 | 0 | 0 | 100 |
| CTONG2025496 | 0 | 0 | 0 | 0 | 0 | 100 |
| CTONG2025513 | 0 | 0 | 0 | 0 | 0 | 100 |
| CTONG2025516 | 0 | 0 | 0 | 0 | 0 | 100 |
| CTONG2025610 | 0 | 0 | 0 | 0 | 0 | 100 |
| CTONG2025900 | 0 | 0 | 0 | 0 | 0 | 100 |
| CTONG2026513 | 0 | 0 | 0 | 0 | 0 | 100 |
| CTONG2026770 | 0 | 0 | 0 | 0 | 0 | 100 |
| CTONG2026920 | 0 | 0 | 0 | 0 | 0 | 100 |
| CTONG2026987 | 0 | 0 | 0 | 0 | 0 | 100 |
| CTONG2027150 | 0 | 0 | 0 | 0 | 0 | 100 |
| CTONG2027263 | 0 | 0 | 0 | 0 | 0 | 30.673 |
| CTONG2027327 | 0 | 0 | 0 | 0 | 0 | 52.760 |
| CTONG2027361 | 0 | 0 | 0 | 0 | 0 | 100 |
| CTONG2027591 | 0 | 0 | 0 | 0 | 0 | 100 |
| CTONG2027736 | 0 | 0 | 0 | 0 | 0 | 100 |
| CTONG2027783 | 0 | 0 | 0 | 0 | 0 | 100 |
| CTONG2027828 | 0 | 0 | 0 | 0 | 0 | 100 |
| CTONG2027959 | 0 | 0 | 0 | 0 | 0 | 72.408 |
| CTONG2028097 | 0 | 0 | 0 | 0 | 0 | 100 |
| CTONG2028124 | 0 | 0 | 0 | 0 | 0 | 0.936 |
| CTONG2028208 | 0 | 0 | 0 | 0 | 0 | 6.596 |
| CTONG2028309 | 0 | 0 | 0 | 0 | 0 | 13.671 |
| CTONG2028486 | 0 | 0 | 0 | 0 | 0 | 64.609 |
| CTONG2028518 | 0 | 0 | 0 | 0 | 0 | 100 |
| CTONG2028687 | 0 | 0 | 0 | 0 | 0 | 100 |
| CTONG2028758 | 0 | 0 | 0 | 0 | 0 | 29.955 |
| CTONG3000657 | 0 | 0 | 0 | 0 | 0 | 7.226 |
| CTONG3000686 | 0 | 0 | 0 | 0 | 0 | 100 |
| CTONG3000707 | 0 | 0 | 0 | 0 | 0 | 100 |
| CTONG3000896 | 0 | 0 | 0 | 0 | 0 | 100 |
| CTONG3001123 | 0 | 0 | 0 | 0 | 0 | 14.616 |
| CTONG3001420 | 0 | 0 | 0 | 0 | 0 | 51.138 |
| CTONG3001501 | 0 | 0 | 0 | 0 | 0 | 100 |
| CTONG3001560 | 0 | 0 | 0 | 0 | 0 | 100 |
| CTONG3001605 | 0 | 0 | 0 | 0 | 0 | 100 |
| CTONG3001746 | 0 | 0 | 0 | 0 | 0 | 26.645 |
| CTONG3002020 | 0 | 0 | 0 | 0 | 0 | 100 |
| CTONG3002127 | 0 | 0 | 0 | 0 | 0 | 62.446 |
| CTONG3002370 | 0 | 0 | 0 | 0 | 0 | 100 |
| CTONG3002412 | 0 | 0 | 0 | 0 | 0 | 19.932 |
| CTONG3002433 | 0 | 0 | 0 | 0 | 0 | 100 |
| CTONG3002518 | 0 | 0 | 0 | 0 | 0 | 15.716 |
| CTONG3002552 | 0 | 0 | 0 | 0 | 0 | 100 |
| CTONG3002588 | 0 | 0 | 0 | 0 | 0 | 100 |
| CTONG3002674 | 0 | 0 | 0 | 0 | 0 | 41.611 |
| CTONG3002728 | 0 | 0 | 0 | 0 | 0 | 100 |
| CTONG3002835 | 0 | 0 | 0 | 7.713 | 0 | 4.093 |
| CTONG3002909 | 0 | 0 | 0 | 0 | 0 | 100 |
| CTONG3002947 | 0 | 0 | 0 | 0 | 0 | 100 |
| CTONG3002983 | 0 | 0 | 0 | 0 | 0 | 100 |
| CTONG3003165 | 0 | 0 | 0 | 0 | 0 | 100 |
| CTONG3003179 | 0 | 0 | 0 | 0 | 0 | 100 |
| CTONG3003483 | 0 | 0 | 0 | 0 | 0 | 100 |
| CTONG3003553 | 0 | 0 | 0 | 0 | 0 | 100 |
| CTONG3003598 | 0 | 0 | 0 | 0 | 0 | 100 |
| CTONG3003652 | 0 | 0 | 0 | 0 | 0 | 100 |
| CTONG3003654 | 0 | 0 | 0 | 0 | 0 | 100 |
| CTONG3003669 | 0 | 0 | 0 | 0 | 0 | 100 |
| CTONG3003737 | 0 | 0 | 0 | 0 | 0 | 100 |
| CTONG3003905 | 0 | 0 | 0 | 0 | 0 | 36.474 |
| CTONG3003972 | 0 | 0 | 0 | 0 | 0 | 51.706 |
| CTONG3004072 | 0 | 0 | 0 | 0 | 0 | 36.356 |
| CTONG3004317 | 0 | 0 | 0 | 0 | 0 | 60.963 |
| CTONG3004397 | 0 | 0 | 0 | 0 | 0 | 100 |
| CTONG3004550 | 0 | 0 | 0 | 0 | 0 | 100 |
| CTONG3004576 | 0 | 0 | 0 | 0 | 0 | 62.446 |
| CTONG3004607 | 0 | 0 | 0 | 0 | 0 | 100 |
| CTONG3004712 | 0 | 0 | 0 | 0 | 0 | 100 |
| CTONG3004726 | 0 | 0 | 0 | 0 | 0 | 31.543 |
| CTONG3005325 | 0 | 0 | 0 | 0 | 0 | 100 |
| CTONG3005648 | 0 | 0 | 0 | 0 | 0 | 100 |
| CTONG3005713 | 0 | 0 | 0 | 0 | 0 | 100 |
| CTONG3005803 | 0 | 0 | 0 | 0 | 0 | 100 |
| CTONG3005813 | 0 | 0 | 0 | 0 | 0 | 72.408 |
| CTONG3006067 | 0 | 0 | 0 | 0 | 0 | 74.021 |
| CTONG3006073 | 0 | 0 | 0 | 0 | 0 | 100 |
| CTONG3006186 | 0 | 0 | 0 | 0 | 0 | 100 |
| CTONG3006629 | 0 | 0 | 0 | 0 | 0 | 59.989 |
| CTONG3006650 | 0 | 0 | 0 | 0 | 0 | 100 |
| CTONG3006659 | 0 | 0 | 0 | 0 | 0 | 100 |
| CTONG3007244 | 0 | 0 | 0 | 0 | 0 | 100 |
| CTONG3007444 | 0 | 0 | 0 | 0 | 0 | 100 |
| CTONG3007528 | 0 | 0 | 0 | 0 | 0 | 100 |
| CTONG3007586 | 0 | 0 | 0 | 0 | 0 | 100 |
| CTONG3007870 | 0 | 0 | 0 | 0 | 0 | 100 |
| CTONG3008223 | 0 | 0 | 0 | 0 | 0 | 100 |
| CTONG3008252 | 0 | 0 | 0 | 0 | 0 | 100 |
| CTONG3008258 | 0 | 0 | 0 | 0 | 0 | 100 |
| CTONG3008457 | 0 | 0 | 0 | 0 | 0 | 100 |
| CTONG3008496 | 0 | 0 | 0 | 0 | 0 | 100 |
| CTONG3008566 | 0 | 0 | 0 | 0 | 0 | 100 |
| CTONG3008639 | 0 | 0 | 0 | 0 | 0 | 100 |
| CTONG3008831 | 0 | 0 | 0 | 0 | 0 | 100 |
| CTONG3008894 | 0 | 0 | 0 | 0 | 0 | 2.708 |
| CTONG3008951 | 0 | 0 | 0 | 0 | 0 | 100 |
| CTONG3008959 | 0 | 0 | 0 | 0 | 0 | 100 |
| CTONG3009028 | 0 | 0 | 0 | 0 | 0 | 5.995 |
| CTONG3009227 | 0 | 0 | 0 | 0 | 0 | 100 |
| CTONG3009239 | 0 | 0 | 0 | 0 | 0 | 5.378 |
| CTONG3009287 | 0 | 0 | 0 | 0 | 0 | 100 |
| CTONG3009328 | 0 | 0 | 0 | 0 | 0 | 44.674 |
| CTONG3009385 | 0 | 0 | 0 | 0 | 0 | 100 |
| D30ST2002068 | 0 | 0 | 0 | 1.508 | 0 | 0 |
| D30ST3000169 | 20.553 | 0 | 0 | 0 | 0 | 0 |
| D90ST2002397 | 0 | 0 | 62.163 | 0 | 0 | 0 |
| DFNES1000150 | 0 | 0 | 0 | 0 | 0 | 2.080 |
| DFNES1000185 | 55.393 | 0 | 0 | 0 | 0 | 0 |
| DFNES2000011 | 0 | 11.300 | 0 | 0 | 0 | 0 |
| DFNES2000335 | 0 | 0 | 0 | 0 | 0 | 8.824 |
| DFNES2004371 | 0 | 0 | 0 | 0 | 0 | 5.877 |
| DFNES2005690 | 0 | 0 | 0 | 0 | 0 | 9.456 |
| DFNES2005766 | 0 | 0 | 0 | 12.871 | 0 | 0 |
| DFNES2007299 | 0 | 0 | 0 | 0 | 0 | 6.117 |
| DFNES2007634 | 0 | 18.368 | 0 | 0 | 0 | 0 |
| DFNES2007757 | 0 | 77.810 | 0 | 0 | 0 | 0 |
| FCBBF1000023 | 0 | 0 | 0 | 0 | 0 | 13.102 |
| FCBBF1000024 | 0 | 0 | 0 | 0 | 0 | 12.268 |
| FCBBF1000027 | 0 | 0 | 0 | 0 | 0 | 2.047 |
| FCBBF1000053 | 0 | 0 | 0 | 0 | 0 | 5.041 |
| FCBBF1000061 | 0 | 0 | 0 | 0 | 0 | 8.761 |
| FCBBF1000197 | 0 | 0 | 0 | 0 | 0 | 9.948 |
| FCBBF1000294 | 0 | 0 | 0 | 0 | 0 | 4.011 |
| FCBBF1000466 | 0 | 0 | 0 | 0 | 0 | 5.931 |
| FCBBF1000574 | 0 | 0 | 0 | 0 | 0 | 3.866 |
| FCBBF2000259 | 0 | 0 | 0 | 0 | 0 | 11.138 |
| FCBBF2000885 | 0 | 0 | 0 | 0 | 0 | 17.154 |
| FCBBF3000110 | 0 | 0 | 0 | 0 | 0 | 12.029 |
| FCBBF3001470 | 0 | 28.053 | 0 | 0 | 0 | 2.528 |
| FCBBF3001818 | 0 | 0 | 0 | 0 | 0 | 16.966 |
| FCBBF3003800 | 0 | 0 | 0 | 0 | 0 | 1.467 |
| FCBBF3004261 | 0 | 0 | 0 | 0 | 0 | 16.849 |
| FCBBF3004473 | 0 | 0 | 0 | 0 | 0 | 4.280 |
| FCBBF3005320 | 0 | 0 | 0 | 0 | 0 | 5.081 |
| FCBBF3005497 | 0 | 0 | 0 | 0 | 0 | 1.464 |
| FCBBF3012347 | 0 | 0 | 0 | 0 | 0 | 4.088 |
| FCBBF3016134 | 0 | 0 | 0 | 0 | 0 | 19.389 |
| FCBBF3017024 | 0 | 0 | 0 | 0 | 0 | 9.546 |
| FCBBF3023368 | 0 | 0 | 0 | 0 | 0 | 9.140 |
| FCBBF3023667 | 0 | 0 | 8.826 | 0.932 | 0 | 0 |
| FCBBF3028244 | 0 | 0 | 0 | 0 | 0 | 1.729 |
| FCBBF4000446 | 0 | 0 | 0 | 0 | 0 | 2.798 |
| FCBBF4000500 | 0 | 0 | 0 | 0 | 0 | 16.324 |
| FEBRA2000105 | 0 | 0 | 0 | 15.106 | 0 | 1.336 |
| FEBRA2000210 | 0 | 0 | 0 | 4.963 | 59.337 | 0 |
| FEBRA2000228 | 0 | 0 | 0 | 0 | 0 | 7.357 |
| FEBRA2000377 | 0 | 0 | 0 | 0 | 0 | 18.632 |
| FEBRA2000510 | 0 | 0 | 0 | 0 | 0 | 6.872 |
| FEBRA2000733 | 0 | 0 | 3.733 | 0 | 0 | 0 |
| FEBRA2000740 | 0 | 23.371 | 0 | 0 | 0 | 6.319 |
| FEBRA2001294 | 0 | 0 | 0 | 0 | 0 | 4.501 |
| FEBRA2001581 | 0 | 0 | 0 | 0 | 0 | 7.637 |
| FEBRA2002527 | 0 | 0 | 0 | 0 | 0 | 5.334 |
| FEBRA2002933 | 0 | 0 | 0 | 0 | 0 | 0.689 |
| FEBRA2003100 | 0 | 0 | 0 | 0 | 0 | 1.227 |
| FEBRA2003750 | 0 | 0 | 0 | 0 | 0 | 6.800 |
| FEBRA2003822 | 0 | 0 | 0 | 38.613 | 0 | 0 |
| FEBRA2003833 | 0 | 0 | 0 | 0 | 0 | 6.441 |
| FEBRA2004042 | 0 | 0 | 0 | 0 | 0 | 3.951 |
| FEBRA2004224 | 0 | 0 | 0 | 8.128 | 0 | 0 |
| FEBRA2004412 | 0 | 0 | 0 | 0 | 0 | 10.937 |
| FEBRA2004818 | 0 | 28.140 | 0 | 0 | 0 | 0 |
| FEBRA2005416 | 0 | 0 | 0 | 0 | 0 | 2.000 |
| FEBRA2005476 | 0 | 0 | 0 | 0 | 0 | 14.554 |
| FEBRA2005824 | 0 | 0 | 0 | 0 | 0 | 1.389 |
| FEBRA2005995 | 0 | 0 | 0 | 0 | 0 | 2.611 |
| FEBRA2006112 | 0 | 0 | 0 | 0 | 0 | 12.294 |
| FEBRA2006270 | 0 | 0 | 0 | 0 | 0 | 6.532 |
| FEBRA2006372 | 0 | 0 | 0 | 0 | 0 | 2.501 |
| FEBRA2006401 | 0 | 0 | 5.560 | 0 | 0 | 0 |
| FEBRA2006485 | 0 | 0 | 0 | 21.356 | 0 | 5.666 |
| FEBRA2007544 | 0 | 0 | 0 | 0 | 0 | 4.560 |
| FEBRA2007622 | 0 | 0 | 0 | 0 | 0 | 3.843 |
| FEBRA2007801 | 0 | 0 | 0 | 0 | 0 | 4.598 |
| FEBRA2007880 | 0 | 0 | 0 | 0 | 0 | 7.010 |
| FEBRA2007900 | 0 | 0 | 0 | 0 | 0 | 14.437 |
| FEBRA2008081 | 0 | 0 | 0 | 0 | 0 | 1.319 |
| FEBRA2008087 | 0 | 0 | 0 | 1.258 | 0 | 0 |
| FEBRA2008201 | 0 | 0 | 0 | 16.836 | 0 | 0 |
| FEBRA2008692 | 0 | 0 | 0 | 0 | 0 | 13.709 |
| FEBRA2009022 | 0 | 0 | 0 | 0 | 0 | 12.440 |
| FEBRA2009419 | 0 | 0 | 0 | 0 | 0 | 7.383 |
| FEBRA2013699 | 0 | 0 | 0 | 0 | 0 | 26.659 |
| FEBRA2015085 | 0 | 0 | 0 | 0 | 0 | 5.680 |
| FEBRA2018407 | 0 | 0 | 0 | 0 | 0 | 30.174 |
| FEBRA2019711 | 0 | 0 | 0 | 29.691 | 0 | 0 |
| FEBRA2022055 | 0 | 0 | 0 | 3.736 | 0 | 0 |
| FEBRA2023399 | 0 | 0 | 0 | 0 | 0 | 16.349 |
| FEBRA2025427 | 0 | 0 | 0 | 0 | 0 | 9.234 |
| FEBRA2028516 | 0 | 0 | 0 | 0 | 0 | 4.261 |
| FELIV1000153 | 0 | 0 | 0 | 0 | 0 | 0.614 |
| HCASM1000115 | 0 | 0 | 0 | 0 | 0 | 1.931 |
| HCASM2000016 | 31.346 | 0 | 0 | 0 | 0 | 1.390 |
| HCASM2000363 | 0 | 0 | 15.065 | 0 | 0 | 1.267 |
| HCHON1000030 | 0 | 0 | 0 | 2.547 | 0 | 2.365 |
| HCHON2000028 | 0 | 0 | 0 | 0 | 0 | 2.955 |
| HCHON2000056 | 0 | 0 | 0 | 0 | 0 | 0.528 |
| HCHON2000087 | 0 | 12.785 | 0 | 0 | 0 | 0 |
| HCHON2000160 | 0 | 0 | 0 | 0 | 0 | 3.750 |
| HCHON2000226 | 0 | 0 | 0 | 0 | 0 | 1.502 |
| HCHON2000295 | 0 | 0 | 0 | 0 | 0 | 0.816 |
| HCHON2000364 | 0 | 0 | 9.849 | 0 | 0 | 0 |
| HCHON2000473 | 0 | 0 | 0 | 0 | 0 | 21.805 |
| HCHON2000475 | 0 | 27.507 | 0 | 0 | 0 | 4.958 |
| HCHON2000508 | 0 | 0 | 0 | 0 | 0 | 9.042 |
| HCHON2000626 | 0 | 0 | 26.041 | 0 | 0 | 0 |
| HCHON2000738 | 0 | 0 | 0 | 0 | 0 | 5.546 |
| HCHON2000751 | 0 | 0 | 0 | 0 | 0 | 6.650 |
| HCHON2000826 | 0 | 0 | 0 | 0 | 0 | 1.333 |
| HCHON2001039 | 0 | 0 | 0 | 0 | 0 | 2.078 |
| HCHON2001050 | 0 | 0 | 0 | 0 | 0 | 5.399 |
| HCHON2001200 | 0 | 0 | 0 | 0 | 0 | 4.028 |
| HCHON2001217 | 0 | 0 | 0 | 0 | 0 | 3.877 |
| HCHON2001359 | 0 | 17.594 | 18.861 | 0 | 0 | 4.757 |
| HCHON2001407 | 0 | 0 | 0 | 0 | 0 | 4.412 |
| HCHON2001535 | 0 | 0 | 0 | 0 | 0 | 17.176 |
| HCHON2001598 | 0 | 50.212 | 0 | 0 | 0 | 0 |
| HCHON2004002 | 0 | 0 | 17.393 | 0 | 0 | 1.462 |
| HHDPC1000118 | 0 | 0 | 0 | 0 | 0 | 3.904 |
| HHDPC2000070 | 0 | 0 | 0 | 0 | 0 | 17.924 |
| HHDPC2000149 | 0 | 0 | 0 | 0 | 0 | 8.322 |
| HHDPC2000456 | 0 | 0 | 34.395 | 0 | 0 | 5.784 |
| HHDPC2000572 | 0 | 0 | 0 | 0 | 0 | 12.194 |
| HHDPC2003439 | 21.364 | 0 | 0 | 0 | 0 | 0.474 |
| HHDPC2003983 | 0 | 5.755 | 0 | 0 | 0 | 1.037 |
| HHDPC2007267 | 0 | 0 | 0 | 0 | 0 | 4.547 |
| HLUNG1000055 | 0 | 0 | 0 | 0 | 0 | 2.016 |
| HLUNG1000076 | 0 | 0 | 0 | 5.906 | 0 | 0 |
| HLUNG1000099 | 0 | 0 | 0 | 0 | 0 | 20.103 |
| HLUNG2000004 | 0 | 0 | 0 | 17.758 | 0 | 0 |
| HLUNG2000412 | 0 | 0 | 0 | 0 | 0 | 4.987 |
| HLUNG2001146 | 0 | 0 | 0 | 0 | 0 | 19.527 |
| HLUNG2002811 | 0 | 50.355 | 0 | 0 | 0 | 4.538 |
| HLUNG2004037 | 0 | 0 | 0 | 0 | 0 | 28.434 |
| HLUNG2004159 | 0 | 0 | 0 | 0 | 0 | 10.131 |
| HLUNG2008479 | 0 | 0 | 0 | 0 | 0 | 33.679 |
| HLUNG2008637 | 0 | 34.908 | 0 | 0 | 0 | 0 |
| HLUNG2009225 | 0 | 0 | 0 | 0 | 0 | 14.045 |
| HLUNG2017262 | 0 | 0 | 0 | 0 | 0 | 5.907 |
| HSYRA1000062 | 0 | 0 | 0 | 0 | 0 | 5.585 |
| HSYRA1000119 | 0 | 0 | 0 | 0 | 0 | 1.713 |
| HSYRA1000152 | 0 | 0 | 0 | 0 | 0 | 4.443 |
| HSYRA2000135 | 0 | 0 | 0 | 0 | 0 | 7.367 |
| HSYRA2000159 | 0 | 0 | 0 | 0 | 10.120 | 0.898 |
| HSYRA2000248 | 0 | 0 | 0 | 0 | 0 | 1.266 |
| HSYRA2000255 | 0 | 0 | 0 | 0 | 0 | 2.507 |
| HSYRA2000743 | 0 | 0 | 0 | 0 | 0 | 1.441 |
| HSYRA2001138 | 0 | 0 | 0 | 1.790 | 0 | 0 |
| HSYRA2001225 | 0 | 0 | 0 | 0 | 0 | 2.754 |
| HSYRA2001476 | 0 | 0 | 0 | 3.389 | 0 | 1.798 |
| HSYRA2001567 | 0 | 0 | 0 | 0 | 0 | 4.174 |
| HSYRA2001574 | 0 | 0 | 0 | 5.282 | 0 | 0 |
| HSYRA2001595 | 0 | 0 | 0 | 0 | 0 | 4.244 |
| HSYRA2001621 | 0 | 3.792 | 0 | 0 | 30.806 | 0 |
| HSYRA2001631 | 0 | 0 | 0 | 0 | 0 | 10.860 |
| HSYRA2007650 | 0 | 0 | 0 | 33.393 | 0 | 0 |
| IMR321000193 | 0 | 0 | 0 | 30.814 | 0 | 0 |
| IMR321000219 | 0 | 5.800 | 0 | 0 | 0 | 1.568 |
| IMR321000253 | 0 | 0 | 0 | 0 | 0 | 3.794 |
| IMR322000518 | 0 | 0 | 0 | 21.562 | 0 | 0 |
| IMR322000730 | 0 | 2.832 | 0 | 7.698 | 0 | 0 |
| IMR322000935 | 0 | 0 | 0 | 0 | 0 | 2.789 |
| IMR322001049 | 0 | 0 | 0 | 0 | 0 | 10.789 |
| IMR322001218 | 0 | 0 | 0 | 0 | 0 | 2.085 |
| IMR322001332 | 0 | 0 | 0 | 18.692 | 0 | 0 |
| IMR322001435 | 0 | 0 | 0 | 0 | 0 | 3.500 |
| IMR322001584 | 0 | 0 | 0 | 8.764 | 0 | 0 |
| IMR322001600 | 0 | 0 | 0 | 0 | 0 | 0.300 |
| KIDNE2000085 | 0 | 0 | 0 | 0 | 0 | 1.915 |
| KIDNE2000252 | 0 | 0 | 0 | 0 | 0 | 3.184 |
| KIDNE2000266 | 0 | 0 | 0 | 0 | 0 | 15.685 |
| KIDNE2000403 | 34.697 | 0 | 0 | 0 | 0 | 4.616 |
| KIDNE2000574 | 0 | 0 | 0 | 0 | 0 | 1.394 |
| KIDNE2000777 | 0 | 0 | 0 | 53.588 | 0 | 0 |
| KIDNE2000947 | 0 | 0 | 0 | 0 | 0 | 9.415 |
| KIDNE2001373 | 0 | 0 | 0 | 50.020 | 0 | 0 |
| KIDNE2001467 | 0 | 0 | 0 | 0 | 0 | 2.913 |
| KIDNE2001847 | 0 | 0 | 0 | 0 | 0 | 6.778 |
| KIDNE2002252 | 0 | 0 | 0 | 0 | 0 | 4.060 |
| KIDNE2003316 | 0 | 0 | 0 | 0 | 0 | 14.092 |
| KIDNE2004411 | 0 | 0 | 0 | 0 | 0 | 15.872 |
| KIDNE2005676 | 0 | 0 | 0 | 0 | 0 | 10.481 |
| KIDNE2006014 | 0 | 0 | 0 | 0 | 0 | 5.530 |
| KIDNE2006248 | 0 | 0 | 0 | 0 | 0 | 1.737 |
| KIDNE2007328 | 37.159 | 0 | 0 | 0 | 0 | 3.296 |
| KIDNE2007910 | 0 | 0 | 0 | 0 | 0 | 16.879 |
| KIDNE2008048 | 0 | 0 | 0 | 0 | 0 | 1.998 |
| KIDNE2009367 | 0 | 0 | 0 | 0 | 0 | 2.808 |
| KIDNE2011508 | 0 | 0 | 0 | 0 | 0 | 34.822 |
| KIDNE2012710 | 0 | 0 | 0 | 0 | 0 | 35.274 |
| KIDNE2013845 | 0 | 0 | 0 | 0 | 0 | 2.082 |
| KIDNE2014290 | 0 | 0 | 0 | 0 | 0 | 9.140 |
| KIDNE2014325 | 0 | 0 | 0 | 0 | 0 | 7.046 |
| KIDNE2016371 | 0 | 0 | 0 | 0 | 0 | 16.781 |
| KIDNE2017040 | 0 | 0 | 0 | 23.608 | 0 | 0 |
| LIVER2000446 | 0 | 0 | 0 | 28.879 | 0 | 0 |
| LIVER2002842 | 0 | 0 | 0 | 0 | 0 | 3.556 |
| LIVER2006006 | 0 | 0 | 0 | 0 | 0 | 2.649 |
| MAMGL1000032 | 0 | 0 | 3.569 | 0 | 0 | 0 |
| MAMGL1000035 | 0 | 0 | 0 | 1.414 | 0 | 1.500 |
| MESAN1000080 | 0 | 0 | 31.630 | 0 | 0 | 0 |
| MESAN1000126 | 0 | 0 | 39.561 | 0 | 0 | 0 |
| MESAN2000092 | 0 | 0 | 0 | 0 | 0 | 16.482 |
| MESAN2000149 | 0 | 0 | 0 | 0 | 0 | 4.575 |
| MESAN2000267 | 0 | 8.462 | 9.072 | 0 | 0 | 0 |
| MESAN2000337 | 0 | 0 | 0 | 0 | 0 | 14.768 |
| MESAN2000572 | 0 | 0 | 0 | 0 | 0 | 7.617 |
| MESAN2000815 | 0 | 0 | 0 | 0 | 0 | 29.082 |
| MESAN2001154 | 55.958 | 0 | 0 | 0 | 0 | 0 |
| MESAN2002086 | 0 | 0 | 0 | 0 | 0 | 0.775 |
| MESAN2002790 | 0 | 0 | 0 | 0 | 0 | 9.111 |
| MESAN2002940 | 0 | 0 | 0 | 0 | 0 | 5.924 |
| MESAN2005633 | 0 | 0 | 0 | 0 | 0 | 31.974 |
| MESAN2005766 | 0 | 0 | 0 | 0 | 0 | 1.837 |
| MESAN2005958 | 0 | 0 | 0 | 0 | 0 | 5.016 |
| MESAN2006401 | 0 | 0 | 0 | 0 | 0 | 3.369 |
| MESAN2006563 | 0 | 14.270 | 0 | 0 | 0 | 2.572 |
| MESAN2009019 | 0 | 0 | 0 | 0 | 0 | 14.550 |
| MESAN2011627 | 0 | 0 | 0 | 0 | 0 | 7.010 |
| MESAN2013284 | 0 | 0 | 0 | 0 | 0 | 4.575 |
| MESAN2015515 | 0 | 0 | 58.849 | 0 | 0 | 0 |
| NB9N41000047 | 0 | 0 | 0 | 0 | 0 | 4.895 |
| NB9N41000135 | 0 | 0 | 0 | 0 | 0 | 4.131 |
| NHNPC2000187 | 0 | 0 | 10.025 | 0 | 0 | 0 |
| NHNPC2000206 | 0 | 0 | 0 | 0 | 0 | 8.953 |
| NHNPC2001931 | 0 | 19.725 | 0 | 0 | 0 | 0 |
| NT2NE1000024 | 0 | 0 | 0 | 26.952 | 0 | 7.150 |
| NT2NE1000163 | 0 | 0 | 0 | 0 | 0 | 1.509 |
| NT2NE2000056 | 0 | 0 | 0 | 0 | 0 | 5.122 |
| NT2NE2000174 | 0 | 0 | 0 | 2.679 | 0 | 0 |
| NT2NE2000260 | 0 | 7.489 | 2.676 | 6.785 | 10.140 | 0 |
| NT2NE2000327 | 0 | 0 | 0 | 0 | 0 | 14.057 |
| NT2NE2000384 | 0 | 0 | 0 | 0 | 0 | 4.287 |
| NT2NE2000575 | 10.097 | 0 | 5.326 | 0 | 0 | 1.343 |
| NT2NE2000707 | 0 | 0 | 0 | 0 | 0 | 1.247 |
| NT2NE2000763 | 0 | 0 | 0 | 0 | 0 | 4.470 |
| NT2NE2000913 | 0 | 0 | 0 | 0 | 0 | 13.739 |
| NT2NE2000963 | 0 | 4.741 | 0 | 3.221 | 0 | 0 |
| NT2NE2001176 | 0 | 23.604 | 0 | 0 | 0 | 0 |
| NT2NE2001397 | 0 | 0 | 0 | 0 | 0 | 24.497 |
| NT2NE2001617 | 0 | 0 | 0 | 31.851 | 0 | 0 |
| NT2NE2001793 | 0 | 0 | 0 | 0 | 0 | 5.796 |
| NT2NE2004716 | 0 | 39.665 | 0 | 0 | 0 | 0 |
| NT2NE2005688 | 0 | 0 | 0 | 0 | 0 | 2.782 |
| NT2NE2005720 | 0 | 0 | 0 | 65.954 | 0 | 0 |
| NT2NE2006075 | 0 | 0 | 0 | 0 | 0 | 29.344 |
| NT2NE2016971 | 0 | 0 | 0 | 0 | 0 | 25.501 |
| NT2RI1000048 | 0 | 17.547 | 0 | 0 | 0 | 0 |
| NT2RI1000127 | 0 | 0 | 0 | 0 | 0 | 3.381 |
| NT2RI2000255 | 0 | 0 | 26.517 | 0 | 0 | 2.229 |
| NT2RI2000263 | 0 | 0 | 0 | 0 | 0 | 3.186 |
| NT2RI2000344 | 0 | 0 | 0 | 0 | 0 | 1.508 |
| NT2RI2000578 | 0 | 0 | 0 | 2.348 | 0 | 0.623 |
| NT2RI2000671 | 0 | 0 | 0 | 7.384 | 0 | 1.959 |
| NT2RI2000727 | 10.866 | 0 | 0 | 0 | 0 | 2.891 |
| NT2RI2001342 | 0 | 0 | 0 | 0 | 0 | 2.940 |
| NT2RI2001540 | 4.782 | 4.705 | 1.261 | 1.599 | 0 | 0.106 |
| NT2RI2001657 | 0 | 0 | 0 | 0 | 0 | 0.997 |
| NT2RI2001859 | 0 | 0 | 0 | 0 | 0 | 3.615 |
| NT2RI2002091 | 0.563 | 0.554 | 1.783 | 0.847 | 0 | 1.224 |
| NT2RI2002117 | 0 | 0 | 0 | 0 | 0 | 8.775 |
| NT2RI2002243 | 0 | 0 | 72.871 | 0 | 0 | 0 |
| NT2RI2002316 | 0 | 0 | 0 | 0 | 0 | 1.493 |
| NT2RI2002391 | 0 | 0 | 0 | 0 | 0 | 1.510 |
| NT2RI2002530 | 0 | 0 | 0 | 0 | 0 | 11.243 |
| NT2RI2002654 | 0 | 0 | 0 | 4.124 | 0 | 0 |
| NT2RI2002729 | 0 | 0 | 0 | 0 | 0 | 3.917 |
| NT2RI2002865 | 0 | 0 | 0 | 47.605 | 0 | 0 |
| NT2RI2003019 | 0 | 0 | 0 | 0 | 0 | 19.482 |
| NT2RI2003051 | 0 | 0 | 0 | 0 | 0 | 3.814 |
| NT2RI2003304 | 0 | 0 | 5.814 | 0 | 0 | 4.399 |
| NT2RI2003407 | 0 | 0 | 0 | 0 | 0 | 2.254 |
| NT2RI2003420 | 0 | 0 | 0 | 0 | 0 | 2.868 |
| NT2RI2003695 | 0 | 0 | 0 | 0 | 0 | 2.933 |
| NT2RI2003738 | 0 | 0 | 0 | 0 | 0 | 4.887 |
| NT2RI2004290 | 0 | 0 | 0 | 0 | 0 | 39.735 |
| NT2RI2004304 | 0 | 0 | 0 | 0 | 0 | 7.756 |
| NT2RI2004884 | 0 | 0 | 0 | 0 | 64.855 | 0 |
| NT2RI2005116 | 0 | 0 | 0 | 0 | 0 | 4.762 |
| NT2RI2005315 | 0 | 0 | 0 | 0 | 0 | 50.616 |
| NT2RI2005335 | 0 | 0 | 0 | 0 | 0 | 4.326 |
| NT2RI2005353 | 48.380 | 0 | 0 | 0 | 0 | 0 |
| NT2RI2005358 | 0 | 0 | 0 | 0 | 0 | 38.226 |
| NT2RI2005647 | 0 | 0 | 0 | 0 | 0 | 6.158 |
| NT2RI2005713 | 0 | 30.561 | 0 | 0 | 0 | 5.509 |
| NT2RI2005851 | 0 | 0 | 0 | 0 | 0 | 6.941 |
| NT2RI2006506 | 0 | 0 | 0 | 0 | 0 | 25.091 |
| NT2RI2006647 | 0 | 0 | 0 | 0 | 0 | 8.541 |
| NT2RI2006735 | 0 | 0 | 0 | 0 | 0 | 29.314 |
| NT2RI2006825 | 0 | 0 | 0 | 0 | 0 | 10.213 |
| NT2RI2006853 | 0 | 0 | 0 | 15.747 | 0 | 12.533 |
| NT2RI2006856 | 0 | 0 | 0 | 0 | 39.317 | 0 |
| NT2RI2007133 | 0 | 0 | 8.691 | 0 | 0 | 0 |
| NT2RI2007384 | 0 | 0 | 0 | 0 | 0 | 0.807 |
| NT2RI2007498 | 0 | 0 | 9.034 | 0 | 0 | 0 |
| NT2RI2007572 | 0 | 0 | 0 | 5.246 | 0 | 4.175 |
| NT2RI2007589 | 0 | 0 | 0 | 7.497 | 0 | 0 |
| NT2RI2007593 | 0 | 0 | 0 | 0 | 0 | 6.137 |
| NT2RI2008141 | 0 | 0 | 0 | 0 | 0 | 3.983 |
| NT2RI2008336 | 16.825 | 0 | 0 | 0 | 0 | 0.746 |
| NT2RI2008396 | 0 | 0 | 0 | 0 | 0 | 4.105 |
| NT2RI2008481 | 0 | 0 | 0 | 5.192 | 0 | 0 |
| NT2RI2008714 | 0 | 0 | 0 | 0 | 0 | 27.330 |
| NT2RI2008724 | 0 | 0 | 0 | 0 | 0 | 3.421 |
| NT2RI2008749 | 0 | 0 | 0 | 0 | 0 | 1.053 |
| NT2RI2008942 | 0 | 0 | 0 | 0 | 0 | 1.953 |
| NT2RI2009037 | 0 | 0 | 0 | 0 | 0 | 3.140 |
| NT2RI2009173 | 46.845 | 0 | 0 | 0 | 0 | 4.155 |
| NT2RI2009233 | 0 | 0 | 0 | 0 | 0 | 8.402 |
| NT2RI2009239 | 58.704 | 0 | 0 | 0 | 0 | 0 |
| NT2RI2009406 | 0 | 0 | 0 | 0 | 0 | 5.240 |
| NT2RI2009583 | 0 | 0 | 0 | 0 | 0 | 0.833 |
| NT2RI2012350 | 0 | 0 | 0 | 0 | 33.597 | 0 |
| NT2RI2015342 | 0 | 0 | 0 | 0 | 0 | 14.010 |
| NT2RI2018448 | 0 | 0 | 0 | 44.206 | 0 | 0 |
| NT2RI2018883 | 0 | 0 | 0 | 0 | 0 | 50.616 |
| NT2RI3003738 | 0 | 0 | 0 | 0 | 0 | 33.781 |
| NT2RI3006284 | 0 | 0 | 0 | 0 | 0 | 3.854 |
| NT2RI3006673 | 0 | 0 | 0 | 0 | 0 | 18.588 |
| NT2RI3007543 | 0 | 0 | 0 | 0 | 0 | 7.901 |
| NT2RI3007757 | 0 | 0 | 0 | 0 | 0 | 23.555 |
| NT2RP6000005 | 0 | 0 | 0 | 0 | 0 | 8.193 |
| NT2RP6000017 | 0 | 0 | 0 | 0 | 0 | 2.787 |
| NT2RP6000109 | 0 | 39.469 | 0 | 0 | 0 | 0 |
| NT2RP6000123 | 0 | 0 | 8.672 | 2.748 | 0 | 2.187 |
| NT2RP7000076 | 0 | 0 | 0 | 0 | 0 | 9.081 |
| NT2RP7000086 | 0 | 0 | 0 | 0 | 0 | 23.818 |
| NT2RP7000173 | 0 | 0 | 0 | 0 | 0 | 5.177 |
| NT2RP7000271 | 0 | 0 | 0 | 0 | 0 | 5.649 |
| NT2RP7000477 | 0 | 0 | 0 | 0 | 0 | 18.424 |
| NT2RP7000600 | 0 | 0 | 0 | 0 | 0 | 4.127 |
| NT2RP7000906 | 0 | 0 | 0 | 0 | 0 | 2.430 |
| NT2RP7001166 | 0 | 0 | 0 | 5.251 | 0 | 4.179 |
| NT2RP7001283 | 0 | 0 | 0 | 0 | 0 | 3.145 |
| NT2RP7001475 | 0 | 0 | 0 | 0 | 0 | 32.721 |
| NT2RP7001591 | 0 | 0 | 0 | 0 | 0 | 9.685 |
| NT2RP7001678 | 0 | 0 | 0 | 0 | 0 | 28.969 |
| NT2RP7002376 | 0 | 0 | 0 | 0 | 0 | 22.598 |
| NT2RP7002379 | 0 | 0 | 11.152 | 0 | 0 | 4.688 |
| NT2RP7002449 | 0 | 0 | 0 | 0 | 0 | 3.763 |
| NT2RP7002450 | 0 | 0 | 0 | 0 | 0 | 4.971 |
| NT2RP7002594 | 0 | 0 | 0 | 0 | 0 | 25.442 |
| NT2RP7002619 | 0 | 0 | 0 | 0 | 0 | 4.059 |
| NT2RP7002802 | 0 | 0 | 0 | 0 | 0 | 6.807 |
| NT2RP7002982 | 0 | 0 | 0 | 0 | 0 | 7.240 |
| NT2RP7003261 | 0 | 0 | 0 | 0 | 0 | 27.835 |
| NT2RP7003319 | 0 | 18.855 | 0 | 0 | 0 | 0 |
| NT2RP7003632 | 0 | 0 | 0 | 0 | 0 | 24.263 |
| NT2RP7003647 | 0 | 0 | 0 | 0 | 0 | 6.576 |
| NT2RP7003688 | 0 | 0 | 0 | 0 | 0 | 16.504 |
| NT2RP7003960 | 0 | 0 | 0 | 0 | 0 | 3.086 |
| NT2RP7004123 | 0 | 0 | 0 | 0 | 0 | 3.574 |
| NT2RP7004173 | 0 | 0 | 0 | 5.362 | 0 | 0 |
| NT2RP7004196 | 0 | 0 | 0 | 0 | 0 | 2.440 |
| NT2RP7004348 | 0 | 0 | 0 | 0 | 0 | 4.209 |
| NT2RP7004352 | 0 | 0 | 0 | 0 | 0 | 12.653 |
| NT2RP7004373 | 0 | 0 | 0 | 0 | 0 | 2.192 |
| NT2RP7004559 | 0 | 0 | 0 | 59.840 | 0 | 0 |
| NT2RP7004751 | 0 | 0 | 0 | 0 | 0 | 0.872 |
| NT2RP7004911 | 0 | 0 | 0 | 0 | 0 | 3.283 |
| NT2RP7004925 | 0 | 0 | 0 | 0 | 0 | 2.352 |
| NT2RP7004946 | 0 | 0 | 0 | 0 | 0 | 3.207 |
| NT2RP7005750 | 0 | 0 | 0 | 0 | 0 | 6.664 |
| NT2RP7006075 | 0 | 0 | 0 | 0 | 0 | 4.880 |
| NT2RP7006141 | 0 | 0 | 0 | 5.185 | 0 | 0 |
| NT2RP7006188 | 0 | 0 | 0 | 0 | 0 | 19.396 |
| NT2RP7006490 | 0 | 0 | 0 | 0 | 0 | 3.998 |
| NT2RP7006701 | 0 | 0 | 0 | 0 | 0 | 9.364 |
| NT2RP7006717 | 0 | 0 | 0 | 0 | 0 | 8.578 |
| NT2RP7007154 | 0 | 11.784 | 0 | 0 | 0 | 0 |
| NT2RP7007177 | 0 | 0 | 0 | 0 | 0 | 7.609 |
| NT2RP7007610 | 0 | 0 | 0 | 0 | 0 | 5.390 |
| NT2RP7007842 | 0 | 0 | 0 | 0 | 0 | 3.572 |
| NT2RP7007975 | 0 | 0 | 0 | 0 | 0 | 7.625 |
| NT2RP7008161 | 0 | 0 | 0 | 0 | 0 | 11.694 |
| NT2RP7008167 | 38.118 | 0 | 0 | 0 | 0 | 0 |
| NT2RP7008190 | 0 | 0 | 0 | 0 | 0 | 2.471 |
| NT2RP7008487 | 0 | 0 | 0 | 0 | 0 | 28.500 |
| NT2RP7008543 | 0 | 0 | 0 | 0 | 0 | 10.248 |
| NT2RP7009012 | 0 | 0 | 0 | 0 | 0 | 21.118 |
| NT2RP7009087 | 0 | 0 | 0 | 0 | 0 | 9.049 |
| NT2RP7009108 | 0 | 0 | 19.689 | 0 | 0 | 8.277 |
| NT2RP7009147 | 0 | 0 | 0 | 0 | 0 | 2.967 |
| NT2RP7009149 | 0 | 0 | 26.502 | 0 | 0 | 0 |
| NT2RP7009259 | 0 | 0 | 0 | 0 | 0 | 20.929 |
| NT2RP7009373 | 0 | 0 | 0 | 0 | 0 | 14.964 |
| NT2RP7009482 | 0 | 0 | 0 | 0 | 0 | 2.590 |
| NT2RP7011909 | 0 | 5.857 | 0 | 0 | 0 | 0 |
| NT2RP7014005 | 0 | 0 | 0 | 0 | 0 | 10.836 |
| NTONG1000033 | 0 | 0 | 0 | 0 | 0 | 22.820 |
| NTONG1000098 | 0 | 0 | 0 | 0 | 0 | 3.560 |
| NTONG1000257 | 0 | 0 | 0 | 0 | 0 | 10.249 |
| NTONG1000264 | 0 | 0 | 0 | 0 | 0 | 1.815 |
| NTONG2000265 | 0 | 0 | 0 | 0 | 0 | 3.176 |
| NTONG2000977 | 0 | 0 | 21.778 | 0 | 0 | 0 |
| NTONG2001137 | 0 | 23.650 | 0 | 8.035 | 0 | 0 |
| NTONG2001222 | 0 | 17.136 | 0 | 0 | 0 | 0 |
| NTONG2001550 | 0 | 0 | 0 | 0 | 0 | 6.483 |
| NTONG2001567 | 0 | 0 | 6.793 | 6.458 | 0 | 2.856 |
| NTONG2001587 | 0 | 0 | 0 | 0 | 0 | 0.554 |
| NTONG2004918 | 0 | 0 | 0 | 0 | 0 | 12.061 |
| NTONG2005497 | 0 | 0 | 6.071 | 0 | 0 | 0 |
| NTONG2005520 | 0 | 0 | 0 | 0 | 0 | 6.199 |
| NTONG2008483 | 0 | 0 | 0 | 0 | 0 | 2.976 |
| NTONG2008522 | 0 | 0 | 0 | 0 | 0 | 7.299 |
| OCBBF1000086 | 0 | 0 | 0 | 0 | 64.252 | 0 |
| OCBBF1000146 | 0 | 0 | 0 | 0 | 0 | 25.706 |
| OCBBF1000188 | 0 | 0 | 23.069 | 0 | 0 | 3.879 |
| OCBBF1000315 | 0 | 0 | 0 | 0 | 0 | 16.296 |
| OCBBF2000015 | 0 | 0 | 0 | 0 | 0 | 1.474 |
| OCBBF2000522 | 0 | 0 | 0 | 0 | 0 | 4.710 |
| OCBBF2000677 | 0 | 0 | 0 | 0 | 0 | 30.340 |
| OCBBF2000846 | 30.735 | 0 | 0 | 0 | 0 | 0 |
| OCBBF2000986 | 0 | 0 | 21.349 | 0 | 0 | 1.795 |
| OCBBF2001140 | 0 | 0 | 0 | 0 | 0 | 10.530 |
| OCBBF2001492 | 0 | 0 | 15.200 | 0 | 0 | 1.278 |
| OCBBF2001687 | 0 | 39.751 | 0 | 0 | 0 | 0 |
| OCBBF2002086 | 0 | 0 | 0 | 0 | 0 | 6.001 |
| OCBBF2005956 | 0 | 0 | 0 | 0 | 0 | 5.287 |
| OCBBF2006148 | 0 | 0 | 0 | 0 | 0 | 12.613 |
| OCBBF2006151 | 0 | 0 | 0 | 0 | 0 | 10.738 |
| OCBBF2007354 | 0 | 0 | 0 | 0 | 0 | 1.871 |
| OCBBF2007829 | 0 | 0 | 0 | 0 | 0 | 1.023 |
| OCBBF2007892 | 0 | 0 | 0 | 29.908 | 0 | 0 |
| OCBBF2008366 | 0 | 0 | 0 | 84.965 | 0 | 0 |
| OCBBF2009242 | 0 | 0 | 0 | 0 | 0 | 5.274 |
| OCBBF2009424 | 0 | 0 | 0 | 0 | 0 | 13.516 |
| OCBBF2010830 | 0 | 0 | 0 | 0 | 0 | 8.194 |
| OCBBF2010858 | 0 | 0 | 0 | 0 | 0 | 12.407 |
| OCBBF2011073 | 0 | 0 | 0 | 0 | 0 | 6.355 |
| OCBBF2011759 | 0 | 0 | 0 | 0 | 0 | 1.741 |
| OCBBF2013843 | 0 | 0 | 0 | 0 | 0 | 0.995 |
| OCBBF2017791 | 0 | 0 | 0 | 0 | 0 | 2.386 |
| OCBBF2018012 | 0 | 0 | 62.179 | 0 | 0 | 0 |
| OCBBF2018362 | 0 | 0 | 0 | 0 | 0 | 42.846 |
| OCBBF2018827 | 0 | 0 | 0 | 0 | 0 | 9.139 |
| OCBBF2018873 | 0 | 0 | 0 | 0 | 0 | 2.581 |
| OCBBF2027148 | 0 | 0 | 0 | 0 | 0 | 43.340 |
| OCBBF3001333 | 0 | 0 | 0 | 22.556 | 0 | 0 |
| OCBBF3004487 | 0 | 0 | 0 | 0 | 0 | 8.439 |
| PEBLM1000174 | 0 | 0 | 0 | 0 | 0 | 8.907 |
| PEBLM2000222 | 0 | 12.004 | 0 | 0 | 0 | 0 |
| PEBLM2000395 | 0 | 0 | 0 | 0 | 0 | 3.390 |
| PEBLM2004452 | 0 | 0 | 0 | 0 | 0 | 15.083 |
| PEBLM2004666 | 0 | 0 | 0 | 0 | 0 | 6.920 |
| PEBLM2007188 | 0 | 0 | 0 | 0 | 0 | 16.259 |
| PEBLM2007774 | 0 | 0 | 0 | 4.229 | 0 | 0 |
| PEBLM2008106 | 0 | 0 | 0 | 0 | 0 | 19.858 |
| PERIC2007068 | 0 | 0 | 0 | 0 | 0 | 2.874 |
| PLACE5000013 | 0 | 0 | 35.166 | 0 | 0 | 0 |
| PLACE5000159 | 0 | 0 | 0 | 0 | 0 | 4.233 |
| PLACE6000145 | 0 | 0 | 22.959 | 0 | 0 | 0 |
| PLACE6000379 | 0 | 0 | 0 | 2.480 | 0 | 1.645 |
| PLACE6000414 | 0 | 0 | 0 | 0 | 0 | 14.320 |
| PLACE6001064 | 0 | 0 | 0 | 0 | 0 | 1.069 |
| PLACE6001281 | 0 | 0 | 0 | 0 | 0 | 6.412 |
| PLACE6002084 | 15.511 | 0 | 0 | 0 | 0 | 1.376 |
| PLACE6002102 | 0 | 0 | 0 | 0 | 0 | 7.892 |
| PLACE6003383 | 0 | 0 | 26.442 | 0 | 0 | 0 |
| PLACE6003393 | 0 | 0 | 0 | 0 | 0 | 14.143 |
| PLACE6003745 | 0 | 0 | 0 | 0 | 0 | 9.787 |
| PLACE6004101 | 0 | 92.071 | 0 | 0 | 0 | 0 |
| PLACE6004312 | 0 | 12.463 | 0 | 0 | 0 | 1.123 |
| PLACE6005294 | 0 | 0 | 0 | 0 | 0 | 22.427 |
| PLACE6006418 | 0 | 0 | 0 | 38.926 | 0 | 10.327 |
| PLACE6006549 | 0 | 16.501 | 0 | 0 | 0 | 0 |
| PLACE6010077 | 0 | 0 | 19.288 | 0 | 0 | 0 |
| PLACE6013222 | 0 | 0 | 0 | 0 | 0 | 9.178 |
| PLACE6018487 | 0 | 0 | 0 | 4.685 | 0 | 0 |
| PLACE7004103 | 0 | 0 | 0 | 0 | 0 | 12.044 |
| PROST1000033 | 0 | 0 | 0 | 0 | 0 | 20.520 |
| PROST1000097 | 0 | 0 | 0 | 0 | 0 | 1.095 |
| PROST1000199 | 0 | 15.099 | 0 | 0 | 0 | 0 |
| PROST1000220 | 0 | 14.031 | 0 | 0 | 0 | 1.265 |
| PROST1000226 | 0 | 0 | 0 | 0 | 0 | 3.332 |
| PROST1000272 | 0 | 0 | 69.900 | 0 | 0 | 0 |
| PROST1000343 | 0 | 0 | 0 | 0 | 0 | 10.582 |
| PROST1000526 | 0 | 0 | 0 | 0 | 0 | 4.706 |
| PROST2000138 | 0 | 1.791 | 3.840 | 1.217 | 0 | 2.906 |
| PROST2000165 | 0 | 0 | 0 | 0 | 0 | 17.008 |
| PROST2000176 | 0 | 0 | 0 | 17.819 | 0 | 0 |
| PROST2000284 | 0 | 0 | 0 | 0 | 0 | 13.045 |
| PROST2001521 | 0 | 0 | 0 | 8.530 | 0 | 0 |
| PROST2002651 | 0 | 0 | 0 | 0 | 0 | 5.570 |
| PROST2003232 | 0 | 0 | 0 | 0 | 0 | 1.271 |
| PROST2004095 | 0 | 0 | 0 | 0 | 0 | 3.487 |
| PROST2004270 | 0 | 12.152 | 0 | 0 | 0 | 4.381 |
| PROST2004416 | 0 | 0 | 0 | 4.774 | 0 | 1.266 |
| PROST2005039 | 0 | 0 | 0 | 0 | 0 | 25.668 |
| PROST2005604 | 0 | 0 | 0 | 0 | 0 | 17.381 |
| PROST2006060 | 0 | 0 | 0 | 46.875 | 0 | 0 |
| PROST2006260 | 0 | 0 | 0 | 0 | 0 | 30.337 |
| PROST2007612 | 0 | 0 | 0 | 0 | 0 | 5.948 |
| PROST2008271 | 0 | 0 | 0 | 0 | 0 | 7.299 |
| PROST2008360 | 0 | 0 | 0 | 12.636 | 0 | 0 |
| PROST2008489 | 0 | 0 | 14.347 | 0 | 0 | 0 |
| PROST2010782 | 0 | 0 | 0 | 0 | 0 | 0.453 |
| PROST2011410 | 0 | 81.943 | 0 | 0 | 0 | 0 |
| PROST2011577 | 0 | 0 | 22.797 | 0 | 0 | 0 |
| PROST2012140 | 0 | 0 | 0 | 0 | 0 | 34.435 |
| PROST2017529 | 0 | 52.453 | 0 | 0 | 0 | 0 |
| PROST2017692 | 0 | 0 | 0 | 0 | 0 | 1.373 |
| PROST2018090 | 0 | 44.955 | 0 | 0 | 0 | 0 |
| PUAEN1000065 | 0 | 0 | 0 | 4.702 | 0 | 0 |
| PUAEN1000081 | 0 | 0 | 0 | 0 | 0 | 24.817 |
| PUAEN1000164 | 0 | 0 | 0 | 0 | 0 | 2.224 |
| PUAEN2000312 | 0 | 0 | 0 | 0 | 0 | 16.489 |
| PUAEN2000374 | 0 | 0 | 0 | 0 | 0 | 1.788 |
| PUAEN2000535 | 0 | 0 | 0 | 0 | 0 | 2.996 |
| PUAEN2003018 | 0 | 0 | 0 | 0 | 0 | 6.243 |
| PUAEN2009174 | 0 | 0 | 62.793 | 0 | 0 | 0 |
| PUAEN2009348 | 0 | 0 | 0 | 55.441 | 0 | 0 |
| RECTM2000433 | 0 | 0 | 0 | 24.395 | 0 | 0 |
| SKMUS1000022 | 17.481 | 0 | 0 | 0 | 0 | 1.550 |
| SKMUS1000138 | 0 | 0 | 0 | 0.674 | 0 | 0 |
| SKMUS1000176 | 0 | 0 | 0 | 0 | 72.419 | 0 |
| SKMUS1000177 | 0 | 0 | 0 | 16.579 | 0 | 8.797 |
| SKMUS1000186 | 0 | 0 | 0 | 0 | 0 | 1.495 |
| SKMUS2000361 | 44.913 | 0 | 0 | 0 | 0 | 0 |
| SKMUS2000821 | 0 | 16.403 | 0 | 0 | 0 | 0 |
| SKMUS2000954 | 0 | 0 | 0 | 0 | 0 | 3.166 |
| SKMUS2001454 | 0 | 0 | 0 | 0 | 0 | 0.629 |
| SKMUS2001528 | 0 | 0 | 0 | 8.812 | 0 | 0 |
| SKMUS2001634 | 0 | 0 | 0 | 0 | 0 | 7.315 |
| SKNMC1000168 | 0 | 0 | 0 | 0 | 0 | 1.560 |
| SKNMC1000229 | 0 | 0 | 0 | 0 | 0 | 19.583 |
| SKNMC1000251 | 0 | 0 | 0 | 0 | 0 | 26.129 |
| SKNMC2000065 | 0 | 0 | 0 | 0 | 0 | 4.021 |
| SKNMC2000097 | 0 | 0 | 0 | 0 | 0 | 1.660 |
| SKNMC2000305 | 0 | 0 | 0 | 0 | 0 | 6.561 |
| SKNMC2000322 | 0 | 29.184 | 0 | 0 | 0 | 0 |
| SKNMC2000635 | 0 | 0 | 11.921 | 0 | 0 | 1.670 |
| SKNMC2000649 | 0 | 0 | 0 | 0 | 0 | 1.699 |
| SKNMC2000698 | 0 | 0 | 0 | 0 | 0 | 11.541 |
| SKNMC2000877 | 0 | 0 | 0 | 0 | 0 | 8.828 |
| SKNMC2003639 | 0 | 0 | 0 | 0 | 0 | 19.285 |
| SKNSH2000347 | 0 | 0 | 0 | 0 | 0 | 8.358 |
| SKNSH2000716 | 0 | 0 | 0 | 0 | 0 | 3.228 |
| SKNSH2000819 | 0 | 0 | 0 | 0 | 0 | 4.636 |
| SKNSH2002325 | 0 | 0 | 0 | 26.801 | 0 | 0 |
| SKNSH2005061 | 71.198 | 0 | 0 | 0 | 0 | 0 |
| SMINT1000131 | 0 | 0 | 20.288 | 0 | 0 | 0 |
| SMINT2000018 | 0 | 0 | 0 | 0 | 0 | 15.683 |
| SMINT2000441 | 0 | 0 | 0 | 0 | 0 | 4.203 |
| SMINT2000468 | 0 | 0 | 0 | 0 | 0 | 12.784 |
| SMINT2001222 | 0 | 85.415 | 0 | 0 | 0 | 0 |
| SMINT2002328 | 0 | 0 | 0 | 0 | 0 | 34.547 |
| SMINT2003074 | 0 | 0 | 0 | 0 | 0 | 5.106 |
| SMINT2003551 | 0 | 0 | 0 | 0 | 0 | 25.838 |
| SMINT2008917 | 0 | 0 | 0 | 0 | 0 | 20.839 |
| SMINT2008921 | 0 | 0 | 0 | 0 | 0 | 2.067 |
| SMINT2009216 | 0 | 0 | 0 | 38.861 | 0 | 0 |
| SMINT2010200 | 0 | 0 | 0 | 66.550 | 0 | 0 |
| SMINT2012291 | 0 | 0 | 0 | 0 | 0 | 8.589 |
| SMINT2017964 | 0 | 0 | 0 | 34.030 | 0 | 0 |
| SPLEN1000166 | 0 | 0 | 0 | 0 | 0 | 2.105 |
| SPLEN2000072 | 0 | 0 | 0 | 42.162 | 0 | 0 |
| SPLEN2000505 | 0 | 0 | 0 | 0 | 0 | 1.570 |
| SPLEN2001141 | 0 | 0 | 0 | 6.630 | 0 | 0 |
| SPLEN2002467 | 0 | 0 | 0 | 0 | 0 | 6.068 |
| SPLEN2002493 | 0 | 0 | 0 | 0 | 0 | 3.224 |
| SPLEN2004078 | 0 | 0 | 0 | 0 | 0 | 27.833 |
| SPLEN2004555 | 0 | 0 | 0 | 0 | 0 | 7.930 |
| SPLEN2004773 | 0 | 0 | 0 | 0 | 0 | 2.651 |
| SPLEN2006133 | 0 | 0 | 0 | 0 | 0 | 3.534 |
| SPLEN2006374 | 0 | 0 | 0 | 0 | 0 | 2.831 |
| SPLEN2007498 | 0 | 0 | 0 | 0 | 0 | 4.874 |
| SPLEN2009970 | 0 | 0 | 0 | 0 | 0 | 9.499 |
| SPLEN2010195 | 0 | 0 | 0 | 0 | 0 | 4.336 |
| SPLEN2010986 | 0 | 0 | 0 | 0 | 0 | 37.509 |
| SPLEN2011758 | 0 | 0 | 0 | 0 | 0 | 9.782 |
| SPLEN2012523 | 44.843 | 0 | 0 | 0 | 0 | 0 |
| SPLEN2012571 | 0 | 0 | 0 | 0 | 0 | 5.921 |
| SPLEN2014911 | 0 | 0 | 0 | 0 | 0 | 3.930 |
| SPLEN2016139 | 53.814 | 0 | 0 | 0 | 0 | 0 |
| SPLEN2017918 | 0 | 0 | 0 | 0 | 0 | 5.094 |
| SPLEN2019077 | 0 | 0 | 0 | 37.244 | 0 | 0 |
| SPLEN2019092 | 0 | 0 | 0 | 0 | 0 | 51.706 |
| SPLEN2019169 | 0 | 0 | 0 | 0 | 0 | 10.544 |
| SPLEN2022522 | 59.537 | 0 | 0 | 0 | 0 | 0 |
| SPLEN2027780 | 0 | 0 | 0 | 0 | 0 | 51.706 |
| SPLEN2028466 | 0 | 17.097 | 0 | 0 | 0 | 2.311 |
| SPLEN2029912 | 0 | 0 | 0 | 0 | 0 | 6.085 |
| SPLEN2031547 | 0 | 0 | 0 | 0 | 0 | 2.932 |
| SPLEN2034448 | 0 | 0 | 0 | 0 | 0 | 9.657 |
| SPLEN2038473 | 0 | 0 | 0 | 0 | 0 | 25.620 |
| SPLEN2038834 | 0 | 0 | 0 | 0 | 0 | 8.628 |
| STOMA2000121 | 0 | 25.183 | 0 | 0 | 0 | 0 |
| STOMA2000482 | 0 | 0 | 0 | 0 | 0 | 7.180 |
| STOMA2002052 | 0 | 0 | 0 | 0 | 0 | 10.359 |
| STOMA2002166 | 0 | 0 | 0 | 4.107 | 0 | 0 |
| SYNOV1000124 | 0 | 0 | 0 | 0 | 0 | 1.036 |
| SYNOV2001212 | 0 | 0 | 0 | 76.498 | 0 | 0 |
| SYNOV2001239 | 0 | 0 | 6.951 | 2.203 | 0 | 0 |
| SYNOV2001468 | 0 | 0 | 0 | 64.073 | 0 | 0 |
| SYNOV2020463 | 0 | 90.549 | 0 | 0 | 0 | 0 |
| SYNOV4001153 | 0 | 0 | 0 | 0 | 0 | 4.320 |
| SYNOV4007671 | 0 | 0 | 0 | 5.146 | 0 | 1.365 |
| SYNOV4009575 | 0 | 0 | 0 | 0 | 0 | 8.924 |
| T1ESE2000904 | 0 | 0 | 0 | 0 | 0 | 4.300 |
| TBAES2000352 | 0 | 0 | 0 | 49.950 | 0 | 0 |
| TBAES2009387 | 0 | 0 | 0 | 3.753 | 0 | 0 |
| TESOP2002273 | 0 | 0 | 0 | 0 | 0 | 7.560 |
| TESOP2007384 | 0 | 0 | 0 | 0 | 0 | 37.388 |
| TESTI1000023 | 0 | 0 | 0 | 0 | 0 | 4.686 |
| TESTI1000138 | 0 | 0 | 0 | 0 | 0 | 55.597 |
| TESTI1000168 | 0 | 0 | 0 | 0 | 0 | 5.837 |
| TESTI2000184 | 0 | 0 | 0 | 0 | 0 | 8.422 |
| TESTI2000784 | 0 | 0 | 0 | 0 | 0 | 8.466 |
| TESTI2001099 | 0 | 0 | 0 | 0 | 0 | 12.778 |
| TESTI2001269 | 0 | 0 | 0 | 0 | 0 | 2.033 |
| TESTI2001511 | 0 | 0 | 38.759 | 0 | 0 | 0 |
| TESTI2002223 | 0 | 0 | 0 | 0 | 0 | 51.220 |
| TESTI2002365 | 0 | 0 | 0 | 0 | 0 | 4.068 |
| TESTI2003005 | 0 | 0 | 0 | 0 | 0 | 5.659 |
| TESTI2003413 | 0 | 0 | 0 | 0 | 0 | 9.699 |
| TESTI2004080 | 0 | 0 | 0 | 0 | 0 | 18.180 |
| TESTI2004295 | 0 | 0 | 0 | 0 | 0 | 30.503 |
| TESTI2004737 | 0 | 0 | 0 | 0 | 0 | 3.299 |
| TESTI2005564 | 0 | 0 | 0 | 0 | 0 | 6.821 |
| TESTI2005743 | 0 | 0 | 0 | 0 | 0 | 4.091 |
| TESTI2005767 | 0 | 0 | 0 | 0 | 0 | 5.094 |
| TESTI2005784 | 0 | 0 | 0 | 0 | 0 | 5.540 |
| TESTI2005788 | 0 | 0 | 0 | 0 | 0 | 8.614 |
| TESTI2006051 | 0 | 0 | 0 | 5.946 | 0 | 0 |
| TESTI2006453 | 0 | 0 | 0 | 0 | 0 | 6.270 |
| TESTI2006872 | 0 | 0 | 0 | 0 | 0 | 10.235 |
| TESTI2007211 | 0 | 0 | 0 | 0 | 0 | 6.152 |
| TESTI2007311 | 0 | 0 | 0 | 0 | 0 | 14.331 |
| TESTI2007998 | 0 | 0 | 0 | 0 | 0 | 5.890 |
| TESTI2008240 | 0 | 0 | 11.080 | 0 | 0 | 0.466 |
| TESTI2009402 | 0 | 0 | 0 | 0 | 0 | 17.075 |
| TESTI2009423 | 0 | 0 | 0 | 0 | 0 | 30.119 |
| TESTI2009474 | 0 | 0 | 0 | 0 | 0 | 3.763 |
| TESTI2009520 | 0 | 0 | 0 | 0 | 0 | 12.571 |
| TESTI2009577 | 0 | 0 | 0 | 0 | 0 | 9.876 |
| TESTI2010513 | 0 | 0 | 0 | 0 | 0 | 4.391 |
| TESTI2012104 | 0 | 0 | 0 | 0 | 0 | 18.132 |
| TESTI2012778 | 0 | 0 | 0 | 0 | 0 | 2.409 |
| TESTI2015246 | 0 | 0 | 0 | 0 | 0 | 22.051 |
| TESTI2015626 | 0 | 0 | 0 | 2.309 | 0 | 0 |
| TESTI2017954 | 0 | 0 | 0 | 0 | 0 | 2.468 |
| TESTI2018014 | 0 | 0 | 0 | 0 | 0 | 7.179 |
| TESTI2018462 | 0 | 0 | 0 | 0 | 0 | 5.263 |
| TESTI2018611 | 0 | 0 | 0 | 0 | 0 | 1.957 |
| TESTI2019729 | 0 | 0 | 0 | 0 | 0 | 74.021 |
| TESTI2021124 | 0 | 0 | 0 | 0 | 0 | 10.645 |
| TESTI2024844 | 48.116 | 23.675 | 0 | 0 | 0 | 0 |
| TESTI2025422 | 0 | 51.293 | 0 | 0 | 0 | 0 |
| TESTI2028296 | 0 | 50.959 | 0 | 0 | 0 | 0 |
| TESTI2034940 | 0 | 0 | 0 | 0 | 21.867 | 0 |
| TESTI2044276 | 0 | 0 | 0 | 0 | 0 | 8.832 |
| TESTI2045155 | 0 | 0 | 0 | 80.865 | 0 | 0 |
| TESTI2046456 | 19.441 | 0 | 10.254 | 0 | 0 | 4.311 |
| TESTI2052693 | 0 | 0 | 0 | 23.507 | 0 | 0 |
| TESTI4000014 | 7.500 | 0 | 0 | 0 | 0 | 0.665 |
| TESTI4002202 | 0 | 0 | 0 | 0 | 0 | 6.175 |
| TESTI4002799 | 0 | 0 | 0 | 0 | 0 | 1.645 |
| TESTI4003150 | 0 | 0 | 0 | 0 | 0 | 27.660 |
| TESTI4008018 | 0 | 0 | 0 | 0 | 0 | 74.021 |
| TESTI4009286 | 0 | 0 | 0 | 0 | 0 | 1.350 |
| TESTI4010851 | 0 | 0 | 0 | 0 | 0 | 1.018 |
| TESTI4013441 | 0 | 0 | 10.424 | 0 | 0 | 0.876 |
| TESTI4013474 | 43.396 | 0 | 0 | 0 | 0 | 0 |
| TESTI4013675 | 0 | 0 | 0 | 0 | 0 | 18.389 |
| TESTI4037156 | 0 | 0 | 0 | 1.943 | 0 | 0 |
| TESTI4038099 | 0 | 0 | 0 | 0 | 0 | 41.085 |
| TESTI4038284 | 0 | 0 | 0 | 0 | 0 | 3.430 |
| THYMU1000002 | 0 | 0 | 0 | 4.155 | 0 | 1.102 |
| THYMU1000016 | 0 | 7.986 | 0 | 0 | 0 | 0.720 |
| THYMU1000041 | 0 | 0 | 0 | 0 | 0 | 13.287 |
| THYMU1000109 | 0 | 0 | 0 | 5.525 | 0 | 0 |
| THYMU1000329 | 0 | 0 | 0 | 0 | 0 | 9.927 |
| THYMU1000374 | 0 | 0 | 0 | 0 | 0 | 2.790 |
| THYMU1000428 | 0 | 0 | 0 | 0 | 0 | 1.059 |
| THYMU1000490 | 0 | 0 | 0 | 0 | 0 | 32.041 |
| THYMU2000382 | 0 | 12.526 | 0 | 0 | 0 | 0 |
| THYMU2000684 | 0 | 0 | 0 | 0 | 0 | 7.755 |
| THYMU2000946 | 0 | 0 | 0 | 0 | 0 | 5.390 |
| THYMU2001018 | 0 | 0 | 0 | 0 | 0 | 16.822 |
| THYMU2002583 | 0 | 0 | 0 | 0 | 0 | 1.931 |
| THYMU2003012 | 0 | 0 | 0 | 0 | 0 | 6.529 |
| THYMU2003046 | 0 | 0 | 0 | 0 | 0 | 3.580 |
| THYMU2003419 | 0 | 0 | 0 | 0 | 42.154 | 3.741 |
| THYMU2003542 | 0 | 44.660 | 0 | 0 | 0 | 0 |
| THYMU2003891 | 6.128 | 0 | 0 | 0 | 0 | 0 |
| THYMU2003981 | 0 | 0 | 0 | 0 | 0 | 0.973 |
| THYMU2004344 | 0 | 0 | 0 | 0 | 0 | 2.379 |
| THYMU2004410 | 0 | 0 | 0 | 0 | 0 | 3.569 |
| THYMU2004906 | 0 | 0 | 0 | 0 | 0 | 23.344 |
| THYMU2005546 | 0 | 0 | 0 | 0 | 0 | 2.037 |
| THYMU2006505 | 0 | 0 | 0 | 0 | 0 | 2.543 |
| THYMU2006813 | 0 | 0 | 0 | 0 | 0 | 2.947 |
| THYMU2009658 | 0 | 0 | 0 | 2.459 | 0 | 0 |
| THYMU2010094 | 0 | 0 | 0 | 0 | 0 | 10.849 |
| THYMU2011308 | 0 | 0 | 92.999 | 0 | 0 | 0 |
| THYMU2012002 | 0 | 0 | 0 | 0 | 0 | 47.289 |
| THYMU2012104 | 0 | 0 | 0 | 0 | 0 | 50.388 |
| THYMU2012690 | 0 | 0 | 86.085 | 0 | 0 | 0 |
| THYMU2013916 | 0 | 0 | 0 | 0 | 0 | 8.263 |
| THYMU2014599 | 0 | 0 | 0 | 0 | 0 | 52.760 |
| THYMU2018126 | 0 | 0 | 0 | 5.108 | 0 | 0 |
| THYMU2027125 | 0 | 3.699 | 0 | 1.257 | 15.025 | 0 |
| THYMU2027975 | 0 | 82.600 | 0 | 0 | 0 | 0 |
| THYMU2031341 | 0 | 71.304 | 0 | 0 | 0 | 0 |
| THYMU2031847 | 0 | 0 | 0 | 0 | 0 | 69.076 |
| THYMU2033308 | 0 | 0 | 0 | 0 | 0 | 26.010 |
| THYMU3013386 | 0 | 0 | 0 | 0 | 62.834 | 0 |
| THYMU3021586 | 0 | 0 | 0 | 0 | 0 | 7.693 |
| TKIDN2015423 | 0 | 0 | 0 | 0 | 0 | 1.186 |
| TLIVE2002046 | 0 | 0 | 0 | 0 | 0 | 3.380 |
| TRACH2000079 | 0 | 0 | 0 | 0 | 0 | 1.009 |
| TRACH2000248 | 0 | 28.835 | 0 | 0 | 0 | 1.299 |
| TRACH2000321 | 0 | 0 | 0 | 0 | 0 | 1.830 |
| TRACH2000461 | 0 | 0 | 0 | 0 | 0 | 6.387 |
| TRACH2000472 | 0 | 0 | 0 | 11.989 | 0 | 1.590 |
| TRACH2000862 | 0 | 0 | 0 | 0 | 0 | 53.910 |
| TRACH2000926 | 0 | 0 | 0 | 0 | 0 | 5.197 |
| TRACH2000972 | 0 | 0 | 0 | 0 | 0 | 1.104 |
| TRACH2001021 | 0 | 8.653 | 0 | 0 | 0 | 0 |
| TRACH2001060 | 0 | 0 | 0 | 0 | 0 | 62.446 |
| TRACH2001101 | 0 | 0 | 0 | 0 | 0 | 19.803 |
| TRACH2001289 | 0 | 0 | 0 | 0 | 0 | 1.899 |
| TRACH2001341 | 0 | 0 | 0 | 0 | 0 | 11.871 |
| TRACH2001428 | 0 | 0 | 0 | 0 | 0 | 4.701 |
| TRACH2001432 | 0 | 22.988 | 0 | 0 | 0 | 0 |
| TRACH2001607 | 0 | 0 | 0 | 0 | 0 | 3.741 |
| TRACH2001612 | 0 | 0 | 0 | 0 | 0 | 7.440 |
| TRACH2001810 | 0 | 0 | 0 | 0 | 0 | 2.893 |
| TRACH2002537 | 0 | 0 | 0 | 0 | 0 | 5.182 |
| TRACH2002803 | 0 | 0 | 0 | 0 | 0 | 11.644 |
| TRACH2003323 | 0 | 0 | 0 | 0 | 0 | 13.259 |
| TRACH2005811 | 0 | 0 | 0 | 0 | 0 | 3.928 |
| TRACH2006866 | 0 | 0 | 0 | 34.834 | 0 | 0 |
| TRACH2007059 | 0 | 0 | 0 | 0 | 0 | 62.446 |
| TRACH2007399 | 0 | 0 | 10.435 | 0 | 0 | 0 |
| TRACH2007816 | 0 | 0 | 0 | 0 | 0 | 18.594 |
| TRACH2008472 | 0 | 0 | 0 | 11.015 | 0 | 0 |
| TRACH2009851 | 0 | 0 | 0 | 0 | 0 | 28.006 |
| TRACH2014077 | 0 | 0 | 0 | 0 | 0 | 6.336 |
| TRACH2014124 | 0 | 0 | 0 | 0 | 0 | 1.923 |
| TRACH2015823 | 0 | 0 | 0 | 0 | 0 | 12.223 |
| TRACH2025535 | 0 | 0 | 0 | 16.304 | 0 | 2.163 |
| TRACH3006717 | 0 | 0 | 0 | 0 | 0 | 13.669 |
| TRACH3007479 | 0 | 0 | 47.650 | 0 | 0 | 0 |
| TRACH3007625 | 0 | 0 | 0 | 0 | 0 | 4.379 |
| TRACH3016805 | 0 | 0 | 0 | 0 | 0 | 8.386 |
| TRACH3036932 | 0 | 0 | 0 | 0 | 0 | 27.304 |
| TRACH3038399 | 0 | 0 | 0 | 0 | 0 | 23.161 |
| TSTOM2000315 | 0 | 0 | 0 | 0 | 0 | 4.245 |
| TSTOM2000553 | 0 | 0 | 0 | 0 | 0 | 12.559 |
| UMVEN2000152 | 0 | 16.277 | 0 | 0 | 0 | 0 |
| UMVEN2000453 | 0 | 0 | 0 | 0 | 0 | 2.917 |
| UTERU2000023 | 0 | 0 | 0 | 0 | 0 | 19.067 |
| UTERU2000095 | 0 | 0 | 0 | 0 | 0 | 2.313 |
| UTERU2000263 | 0 | 10.589 | 11.351 | 0 | 0 | 0 |
| UTERU2000300 | 0 | 0 | 0 | 0 | 0 | 9.187 |
| UTERU2000424 | 0 | 0 | 0 | 0 | 0 | 1.582 |
| UTERU2001607 | 0 | 0 | 0 | 0 | 0 | 2.748 |
| UTERU2001658 | 0 | 0 | 0 | 0 | 0 | 16.498 |
| UTERU2003057 | 0 | 0 | 57.297 | 0 | 0 | 0 |
| UTERU2004037 | 0 | 0 | 0 | 15.393 | 0 | 0 |
| UTERU2004197 | 0 | 0 | 0 | 0 | 0 | 7.490 |
| UTERU2004520 | 0 | 0 | 0 | 0 | 0 | 60.963 |
| UTERU2004688 | 0 | 22.977 | 0 | 0 | 0 | 0 |
| UTERU2005593 | 0 | 0 | 0 | 0 | 0 | 2.091 |
| UTERU2005621 | 0 | 0 | 0 | 0 | 0 | 8.397 |
| UTERU2006593 | 0 | 0 | 0 | 0 | 0 | 2.750 |
| UTERU2008085 | 0 | 1.866 | 0 | 6.340 | 0 | 0.168 |
| UTERU2008938 | 0 | 0 | 0 | 0 | 0 | 4.584 |
| UTERU2010417 | 0 | 0 | 0 | 0 | 0 | 2.936 |
| UTERU2011906 | 0 | 0 | 0 | 0 | 0 | 12.702 |
| UTERU2012407 | 0 | 0 | 0 | 0 | 0 | 45.844 |
| UTERU2014898 | 0 | 31.445 | 0 | 0 | 0 | 0 |
| UTERU2015653 | 0 | 0 | 12.924 | 0 | 0 | 0 |
| UTERU2017762 | 0 | 0 | 0 | 0 | 0 | 60.963 |
| UTERU2019096 | 0 | 0 | 0 | 0 | 0 | 4.290 |
| UTERU2023175 | 0 | 0 | 0 | 0 | 0 | 2.773 |
| UTERU2024969 | 0 | 0 | 0 | 0 | 0 | 5.981 |
| UTERU3000298 | 0 | 0 | 0 | 0 | 0 | 8.617 |
| UTERU3005767 | 0 | 24.596 | 0 | 8.356 | 0 | 0 |

Genes whose expression levels were negligibly low in each of the above-described libraries are shown with their clone names below.

3NB691000018, 3NB691000085, 3NB691000113, 3NB691000129, 3NB692000012, 3NB692000029, 3NB692000102, 3NB692000154, 3NB692000276, 3NB692000305, 3NB692000330, 3NB692000374, 3NB692000429, 3NB692000484, 3NB692000529, 3NB692000545, 3NB692000973, 3NB692001002, 3NB692001022, 3NB692001034, 3NB692001040, 3NB692001123, 3NB692001267, 3NB692001288, 3NB692001349, 3NB692001366, 3NB692001408, 3NB692001442, 3NB692001459, 3NB692001471, 3NB692001496, 3NB692001501, 3NB692001507, 3NB692001511, 3NB692001519, 3NB692001520, 3NB692001528, 3NB692001538, 3NB692001548, 3NB692001557, 3NB692001637, 3NB692001719, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064, ADIPS2000069, ADIPS2000088, ADIPS2000245, ADIPS2000425, ADRGL1000002, ADRGL1000018, ADRGL1000033, ADRGL1000038, ADRGL1000065, ADRGL1000067, ADRGL1000111, ADRGL1000144, ADRGL1000147, ADRGL1000160, ADRGL1000165, ADRGL1000182, ADRGL2000042, ADRGL2000056, ADRGL2000064, ADRGL2000074, ADRGL2000085, ADRGL2000097, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001836, ADRGL2001854, ADRGL2002013, ADRGL2002029, ADRGL2002191, ADRGL2002260, ADRGL2002392, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146, ADRGL2009273, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO1000165, ASTRO2000011, ASTRO2000095, ASTRO2000141, ASTRO2000191, ASTRO2000278, ASTRO2000372, ASTRO2000381, ASTRO2000403, ASTRO2000417, ASTRO2000480, ASTRO2000482, ASTRO2000533, ASTRO2000653, ASTRO2000662, ASTRO2000725, ASTRO2000801, ASTRO2000914, ASTRO2001001, ASTRO2001008, ASTRO2001088, ASTRO2001107, ASTRO2001122, ASTRO2001227, ASTRO2001249, ASTRO2001480, ASTRO2001806, ASTRO2002024, ASTRO2002035, ASTRO2002064, ASTRO2002202, ASTRO2002459, ASTRO2002571, ASTRO2002632, ASTRO2002659, ASTRO2002693, ASTRO2002720, ASTRO2002733, ASTRO2002743, ASTRO2002842, ASTRO2003212, ASTRO2003316, ASTRO2003574, ASTRO2003581, ASTRO2003740, ASTRO2003840, ASTRO2003925, ASTRO2003960, ASTRO2004051, ASTRO2004177, ASTRO2004217, ASTRO2004297, ASTRO2004584, ASTRO2004628, ASTRO2004751, ASTRO2004877, ASTRO2004974, ASTRO2005008, ASTRO2005081, ASTRO2005096, ASTRO2005273, ASTRO2005343, ASTRO2005360, ASTRO2005413, ASTRO2005423, ASTRO2005553, ASTRO2005557, ASTRO2005575, ASTRO2005593, ASTRO2005687, ASTRO2005726, ASTRO2005896, ASTRO2005902, ASTRO2006356, ASTRO2006475, ASTRO2006747, ASTRO2006920, ASTRO2006952, ASTRO2007047, ASTRO2007377, ASTRO2007515, ASTRO2007578, ASTRO2007643, ASTRO2007666, ASTRO2007956, ASTRO2007962, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2012552, ASTRO2013124, ASTRO2013585, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014863, ASTRO2014923, ASTRO2014961, ASTRO2015162, ASTRO2015185, ASTRO2015328, ASTRO2016044, ASTRO2016114, ASTRO2016148, ASTRO2016681, ASTRO2016819, ASTRO2016847, ASTRO2016892, ASTRO2017348, ASTRO2017627, ASTRO2018169, ASTRO2018373, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000123, BGGI11000193, BGGI11000285, BGGI12000022, BGGI12000054, BGGI12000067, BGGI12000161, BGGI12000533, BGGI12000544, BGGI12000616, BGGI12000684, BGGI12000693, BGGI12000839, BGGI12001002, BGGI12001075, BGGI12001097, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006830, BLADE2006849, BLADE2006947, BLADE2007033, BLADE2007043, BLADE2007589, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000137, BNGH41000153, BNGH41000169, BNGH41000177, BNGH41000216, BNGH41000235, BNGH42000130, BNGH42000132, BNGH42000405, BNGH42000675, BNGH42000739, BNGH42000791, BNGH42000815, BNGH42000994, BNGH42001247, BNGH42001406, BNGH42001576, BNGH42001724, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42005968, BNGH42006226, BNGH42006234, BNGH42007037, BNGH42007366, BNGH42007594, BNGH42007798, BNGH42008199, BNGH42008510, BNGH42008603, BNGH42008649, BNGH42008832, BNGH42008850, BNGH42008943, BNGH42009021, BNGH42009025, BRACE1000020, BRACE1000042, BRACE1000047, BRACE1000051, BRACE1000065, BRACE1000070, BRACE1000073, BRACE1000084, BRACE1000087, BRACE1000092, BRACE1000093, BRACE1000101, BRACE1000114, BRACE1000115, BRACE1000124, BRACE1000132, BRACE1000159, BRACE1000166, BRACE1000169, BRACE1000186, BRACE1000187, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000331, BRACE2000332, BRACE2000347, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000753, BRACE2000815, BRACE2000885, BRACE2000893, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001143, BRACE2001151, BRACE2001154, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001492, BRACE2001496, BRACE2001497, BRACE2001508, BRACE2001543, BRACE2001564, BRACE2001588, BRACE2001607, BRACE2001626, BRACE2001673, BRACE2001677, BRACE2001692, BRACE2001705, BRACE2001711, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001892, BRACE2001898, BRACE2001923, BRACE2001942, BRACE2001944, BRACE2001954, BRACE2001971, BRACE2001972, BRACE2001973, BRACE2002026, BRACE2002034, BRACE2002050, BRACE2002081, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002392, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002661, BRACE2002676, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002807, BRACE2002812, BRACE2002834, BRACE2002849, BRACE2002857, BRACE2002860, BRACE2002861, BRACE2002884, BRACE2002896, BRACE2002948, BRACE2003037, BRACE2003097, BRACE2003110, BRACE2003168, BRACE2003172, BRACE2003199, BRACE2003210, BRACE2003217, BRACE2003269, BRACE2003285, BRACE2003319, BRACE2003398, BRACE2003420, BRACE2003428, BRACE2003431, BRACE2003512, BRACE2003516, BRACE2003527, BRACE2003539, BRACE2003540, BRACE2003598, BRACE2003609, BRACE2003614, BRACE2003628, BRACE2003639, BRACE2003700, BRACE2003713, BRACE2003766, BRACE2003788, BRACE2003800, BRACE2003802, BRACE2003825, BRACE2003827, BRACE2003847, BRACE2003848, BRACE2003873, BRACE2003885, BRACE2003887, BRACE2003905, BRACE2003928, BRACE2003944, BRACE2003954, BRACE2003982, BRACE2003987, BRACE2005034, BRACE2005047, BRACE2005063, BRACE2005083, BRACE2005090, BRACE2005099, BRACE2005118, BRACE2005124, BRACE2005138, BRACE2005151, BRACE2005160, BRACE2005169, BRACE2005189, BRACE2005193, BRACE2005196, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005433, BRACE2005434, BRACE2005450, BRACE2005457, BRACE2005460, BRACE2005489, BRACE2005518, BRACE2005556, BRACE2005562, BRACE2005624, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006055, BRACE2006075, BRACE2006084, BRACE2006089, BRACE2006102, BRACE2006122, BRACE2006137, BRACE2006143, BRACE2006167, BRACE2006174, BRACE2006240, BRACE2006245, BRACE2006249, BRACE2006258, BRACE2006264, BRACE2006274, BRACE2006281, BRACE2006298, BRACE2006344, BRACE2006354, BRACE2006363, BRACE2006378, BRACE2006380, BRACE2006393, BRACE2006413, BRACE2006417, BRACE2006453, BRACE2006459, BRACE2006488, BRACE2006534, BRACE2006537, BRACE2006540, BRACE2006547, BRACE2006564, BRACE2006587, BRACE2006598, BRACE2006636, BRACE2006685, BRACE2006703, BRACE2006743, BRACE2006753, BRACE2006783, BRACE2006821, BRACE2006833, BRACE2006859, BRACE2006871, BRACE2006909, BRACE2006911, BRACE2006924, BRACE2006935, BRACE2006944, BRACE2006951, BRACE2006982, BRACE2006986, BRACE2007013, BRACE2007064, BRACE2007068, BRACE2007087, BRACE2007089, BRACE2007109, BRACE2007138, BRACE2007174, BRACE2007191, BRACE2007197, BRACE2007201, BRACE2007203, BRACE2007232, BRACE2007254, BRACE2007262, BRACE2007274, BRACE2007281, BRACE2007295, BRACE2007358, BRACE2007396, BRACE2007398, BRACE2007411, BRACE2007447, BRACE2007477, BRACE2007502, BRACE2007503, BRACE2007522, BRACE2007527, BRACE2007538, BRACE2007563, BRACE2007567, BRACE2007621, BRACE2007640, BRACE2007641, BRACE2007646, BRACE2007663, BRACE2007682, BRACE2007685, BRACE2007708, BRACE2007727, BRACE2007749, BRACE2007760, BRACE2007761, BRACE2007764, BRACE2007767, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008097, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008358, BRACE2008361, BRACE2008380, BRACE2008385, BRACE2008399, BRACE2008401, BRACE2008402, BRACE2008410, BRACE2008420, BRACE2008443, BRACE2008473, BRACE2008480, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008706, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008941, BRACE2008948, BRACE2008960, BRACE2008968, BRACE2008999, BRACE2009014, BRACE2009016, BRACE2009031, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009291, BRACE2009293, BRACE2009307, BRACE2009311, BRACE2009318, BRACE2009323, BRACE2009421, BRACE2009428, BRACE2009434, BRACE2009437, BRACE2009483, BRACE2009533, BRACE2009558, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317, BRACE2012510, BRACE2012625, BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017410, BRACE2017438, BRACE2017574, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039624, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004150, BRACE3004205, BRACE3004358, BRACE3004371, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005938, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010438, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000354, BRAMY2000388, BRAMY2000411, BRAMY2000508, BRAMY2000562, BRAMY2000653, BRAMY2000814, BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004387, BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012119, BRAMY2012162, BRAMY2012163, BRAMY2012277, BRAMY2012340, BRAMY2012426, BRAMY2012497, BRAMY2012517, BRAMY2012536, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016325, BRAMY2016327, BRAMY2016386, BRAMY2016706, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019600, BRAMY2019643, BRAMY2019710, BRAMY2019963, BRAMY2019985, BRAMY2019989, BRAMY2020050, BRAMY2020106, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2023939, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026713, BRAMY2026824, BRAMY2026899, BRAMY2026902, BRAMY2026904, BRAMY2026976, BRAMY2027073, BRAMY2027114, BRAMY2027242, BRAMY2027396, BRAMY2027451, BRAMY2027461, BRAMY2027467, BRAMY2027587, BRAMY2027714, BRAMY2027717, BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028783, BRAMY2028796, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002805, BRAMY3002886, BRAMY3002991, BRAMY3003026, BRAMY3003109, BRAMY3003205, BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001913, BRAMY4002575, BRAMY4002628, BRASW1000053, BRASW1000125, BRAWH1000001, BRAWH1000002, BRAWH1000007, BRAWH1000037, BRAWH1000040, BRAWH1000045, BRAWH1000083, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417, BRAWH2000443, BRAWH2000471, BRAWH2000476, BRAWH2000482, BRAWH2000488, BRAWH2000494, BRAWH2000500, BRAWH2000503, BRAWH2000522, BRAWH2000554, BRAWH2000588, BRAWH2000595, BRAWH2000636, BRAWH2000643, BRAWH2000651, BRAWH2000682, BRAWH2000686, BRAWH2000697, BRAWH2000752, BRAWH2000859, BRAWH2000866, BRAWH2000892, BRAWH2000901, BRAWH2000924, BRAWH2000926, BRAWH2000944, BRAWH2000984, BRAWH2001019, BRAWH2001037, BRAWH2001052, BRAWH2001103, BRAWH2001129, BRAWH2001137, BRAWH2001141, BRAWH2001159, BRAWH2001166, BRAWH2001186, BRAWH2001203, BRAWH2001238, BRAWH2001239, BRAWH2001255, BRAWH2001355, BRAWH2001378, BRAWH2001395, BRAWH2001406, BRAWH2001412, BRAWH2001418, BRAWH2001424, BRAWH2001436, BRAWH2001438, BRAWH2001439, BRAWH2001459, BRAWH2001461, BRAWH2001484, BRAWH2001492, BRAWH2001503, BRAWH2001519, BRAWH2001530, BRAWH2001535, BRAWH2001589, BRAWH2001662, BRAWH2001666, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003025, BRAWH2003307, BRAWH2003355, BRAWH2003366, BRAWH2003662, BRAWH2003689, BRAWH2003693, BRAWH2003818, BRAWH2003832, BRAWH2003948, BRAWH2003964, BRAWH2004044, BRAWH2004068, BRAWH2004078, BRAWH2004095, BRAWH2004136, BRAWH2004217, BRAWH2004580, BRAWH2004731, BRAWH2004779, BRAWH2004842, BRAWH2004884, BRAWH2004923, BRAWH2005008, BRAWH2005074, BRAWH2005225, BRAWH2005315, BRAWH2005517, BRAWH2005524, BRAWH2005533, BRAWH2005578, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2006996, BRAWH2007069, BRAWH2007308, BRAWH2007406, BRAWH2007550, BRAWH2007570, BRAWH2007591, BRAWH2007605, BRAWH2007658, BRAWH2007664, BRAWH2007800, BRAWH2007808, BRAWH2007825, BRAWH2007862, BRAWH2007890, BRAWH2008058, BRAWH2008128, BRAWH2008219, BRAWH2008255, BRAWH2008292, BRAWH2008297, BRAWH2008366, BRAWH2008380, BRAWH2008540, BRAWH2008706, BRAWH2008790, BRAWH2008903, BRAWH2008993, BRAWH2009112, BRAWH2009238, BRAWH2009261, BRAWH2009263, BRAWH2009298, BRAWH2009307, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010536, BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014234, BRAWH2014379, BRAWH2014414, BRAWH2014473, BRAWH2014511, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017542, BRAWH2017635, BRAWH2017913, BRAWH2018206, BRAWH2018267, BRAWH2018282, BRAWH2018317, BRAWH2018506, BRAWH2018514, BRAWH2018526, BRAWH2018527, BRAWH2018601, BRAWH2018729, BRAWH2018745, BRAWH2018875, BRAWH2019053, BRAWH2019055, BRAWH2019198, BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002600, BRAWH3002819, BRAWH3003019, BRAWH3003136, BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN1000168, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015402, BRCAN2015464, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2025009, BRCAN2025093, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028040, BRCAN2028319, BRCAN2028338, BRCAN2028355, BRCAN2028378, BRCAN2028460, BRCAN2028545, BRCAN2028634, BRCAN2028702, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019910, BRCOC2019934, BRCOC2020122, BRCOC2020142, BRHIP1000058, BRHIP1000072, BRHIP1000104, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000621, BRHIP2000691, BRHIP2000819, BRHIP2000826, BRHIP2000920, BRHIP2001074, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007741, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011491, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387, BRHIP2014954, BRHIP2015153, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020799, BRHIP2020842, BRHIP2020859, BRHIP2020930, BRHIP2021102, BRHIP2021289, BRHIP2021495, BRHIP2021615, BRHIP2021744, BRHIP2021762, BRHIP2021858, BRHIP2021870, BRHIP2021929, BRHIP2022006, BRHIP2022054, BRHIP2022221, BRHIP2022228, BRHIP2022298, BRHIP2022467, BRHIP2022708, BRHIP2022986, BRHIP2023071, BRHIP2023229, BRHIP2023272, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024398, BRHIP2024549, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000060, BRHIP3000070, BRHIP3000081, BRHIP3000111, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3018784, BRHIP3019643, BRHIP3019824, BRHIP3019880, BRHIP3019956, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000518, BRSSN2000561, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2001869, BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009627, BRSSN2009630, BRSSN2009702, BRSSN2010019, BRSSN2010110, BRSSN2010587, BRSSN2010596, BRSSN2010830, BRSSN2011262, BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014636, BRSSN2014685, BRSSN2015199, BRSSN2015238, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451, BRSTN2013171, BRSTN2013354, BRSTN2013931, BRSTN2014490, BRSTN2014633, BRSTN2014751, BRSTN2014770, BRSTN2015015, BRSTN2015889, BRSTN2015978, BRSTN2016066, BRSTN2016335, BRSTN2016678, BRSTN2016892, BRSTN2016918, BRSTN2016992, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005864, BRTHA2005942, BRTHA2005956, BRTHA2006075, BRTHA2006146, BRTHA2006194, BRTHA2006232, BRTHA2006720, BRTHA2006735, BRTHA2006974, BRTHA2007060, BRTHA2007122, BRTHA2007422, BRTHA2007603, BRTHA2007665, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562, BRTHA2012714, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003490, BRTHA3003704, BRTHA3003736, BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020369, BRTHA3021569, BRTHA3021708, BRTHA3021786, BRTHA3021971, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001670, D3OST2001788, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000195, D3OST3000258, D3OST3000291, D3OST3000388, D3OST3000493, D6OST2000127, D6OST2000169, D6OST2000245, D6OST2000358, D6OST2000464, D6OST2000507, D9OST2000031, D9OST2000278, D9OST2000440, D9OST2000869, D9OST2001319, D9OST2001433, D9OST2002608, D9OST2002673, D9OST2003106, D9OST2003108, D9OST2003137, D9OST2003397, D9OST2003580, D9OST2003594, D9OST2003762, D9OST2003791, D9OST2003989, D9OST2004018, D9OST2004073, D9OST2004417, DFNES1000003, DFNES1000083, DFNES1000107, DFNES2000143, DFNES2000146, DFNES2000153, DFNES2000212, DFNES2000268, DFNES2000292, DFNES2000426, DFNES2000432, DFNES2000443, DFNES2000457, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2002966, DFNES2003081, DFNES2003192, DFNES2003255, DFNES2003742, DFNES2004346, DFNES2004354, DFNES2004383, DFNES2004608, DFNES2004684, DFNES2005266, DFNES2005527, DFNES2005540, DFNES2005779, DFNES2005875, DFNES2006346, DFNES2006944, DFNES2007113, DFNES2007332, DFNES2007641, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000025, FCBBF1000038, FCBBF1000063, FCBBF1000069, FCBBF1000077, FCBBF1000115, FCBBF1000117, FCBBF1000118, FCBBF1000121, FCBBF1000125, FCBBF1000131, FCBBF1000139, FCBBF1000155, FCBBF1000157, FCBBF1000171, FCBBF1000174, FCBBF1000182, FCBBF1000198, FCBBF1000206, FCBBF1000220, FCBBF1000232, FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000367, FCBBF1000374, FCBBF1000376, FCBBF1000377, FCBBF1000395, FCBBF1000397, FCBBF1000403, FCBBF1000412, FCBBF1000425, FCBBF1000437, FCBBF1000449, FCBBF1000476, FCBBF1000506, FCBBF1000509, FCBBF1000546, FCBBF1000550, FCBBF1000556, FCBBF1000563, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF1000760, FCBBF2000038, FCBBF2000087, FCBBF2000094, FCBBF2000105, FCBBF2000123, FCBBF2000195, FCBBF2000199, FCBBF2000214, FCBBF2000232, FCBBF2000276, FCBBF2000291, FCBBF2000362, FCBBF2000379, FCBBF2000473, FCBBF2000502, FCBBF2000566, FCBBF2000576, FCBBF2000591, FCBBF2000677, FCBBF2000678, FCBBF2000685, FCBBF2000733, FCBBF2000782, FCBBF2000808, FCBBF2000815, FCBBF2000825, FCBBF2000940, FCBBF2000951, FCBBF2001001, FCBBF2001088, FCBBF2001105, FCBBF2001116, FCBBF2001183, FCBBF2001188, FCBBF2001211, FCBBF2001238, FCBBF2001243, FCBBF2001291, FCBBF2001299, FCBBF2001309, FCBBF2001400, FCBBF2001427, FCBBF2001480, FCBBF2001529, FCBBF2001538, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002349, FCBBF2002370, FCBBF2002541, FCBBF2002626, FCBBF2002801, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005909, FCBBF2005966, FCBBF2006131, FCBBF2006380, FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000115, FCBBF3000120, FCBBF3000168, FCBBF3000175, FCBBF3000227, FCBBF3000228, FCBBF3000229, FCBBF3000233, FCBBF3000269, FCBBF3000361, FCBBF3000367, FCBBF3000388, FCBBF3000434, FCBBF3000462, FCBBF3000473, FCBBF3000501, FCBBF3000518, FCBBF3000536, FCBBF3000550, FCBBF3000847, FCBBF3001018, FCBBF3001020, FCBBF3001081, FCBBF3001235, FCBBF3001281, FCBBF3001302, FCBBF3001377, FCBBF3001594, FCBBF3001632, FCBBF3001657, FCBBF3001855, FCBBF3001890, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004323, FCBBF3004390, FCBBF3004409, FCBBF3004502, FCBBF3004715, FCBBF3004842, FCBBF3004847, FCBBF3004955, FCBBF3005160, FCBBF3005218, FCBBF3005330, FCBBF3005444, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008153, FCBBF3008159, FCBBF3008251, FCBBF3008311, FCBBF3008362, FCBBF3008382, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009541, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016178, FCBBF3016451, FCBBF3016618, FCBBF3016622, FCBBF3016644, FCBBF3016667, FCBBF3016928, FCBBF3016987, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018796, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019714, FCBBF3019716, FCBBF3019784, FCBBF3019839, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028472, FCBBF3028528, FCBBF3028582, FCBBF3028593, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000529, FCBBF4000548, FCBBF5000041, FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000022, FEBRA1000030, FEBRA1000057, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000129, FEBRA2000220, FEBRA2000253, FEBRA2000282, FEBRA2000286, FEBRA2000297, FEBRA2000311, FEBRA2000321, FEBRA2000333, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000452, FEBRA2000454, FEBRA2000462, FEBRA2000468, FEBRA2000491, FEBRA2000504, FEBRA2000532, FEBRA2000536, FEBRA2000538, FEBRA2000575, FEBRA2000581, FEBRA2000656, FEBRA2000659, FEBRA2000680, FEBRA2000690, FEBRA2000727, FEBRA2000734, FEBRA2000757, FEBRA2000762, FEBRA2000771, FEBRA2000772, FEBRA2000782, FEBRA2000787, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000881, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001245, FEBRA2001267, FEBRA2001334, FEBRA2001351, FEBRA2001383, FEBRA2001438, FEBRA2001440, FEBRA2001487, FEBRA2001492, FEBRA2001523, FEBRA2001561, FEBRA2001571, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001752, FEBRA2001772, FEBRA2001790, FEBRA2001814, FEBRA2001828, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002429, FEBRA2002442, FEBRA2002456, FEBRA2002508, FEBRA2002525, FEBRA2002552, FEBRA2002574, FEBRA2002601, FEBRA2002611, FEBRA2002628, FEBRA2002662, FEBRA2002682, FEBRA2002707, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003897, FEBRA2003907, FEBRA2003926, FEBRA2003930, FEBRA2004023, FEBRA2004026, FEBRA2004029, FEBRA2004053, FEBRA2004056, FEBRA2004091, FEBRA2004110, FEBRA2004191, FEBRA2004202, FEBRA2004219, FEBRA2004237, FEBRA2004268, FEBRA2004293, FEBRA2004325, FEBRA2004329, FEBRA2004331, FEBRA2004443, FEBRA2004485, FEBRA2004490, FEBRA2004538, FEBRA2004592, FEBRA2004620, FEBRA2004628, FEBRA2004651, FEBRA2004767, FEBRA2004813, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005427, FEBRA2005458, FEBRA2005572, FEBRA2005701, FEBRA2005726, FEBRA2005734, FEBRA2005752, FEBRA2005778, FEBRA2005787, FEBRA2005788, FEBRA2005998, FEBRA2006055, FEBRA2006061, FEBRA2006092, FEBRA2006150, FEBRA2006165, FEBRA2006255, FEBRA2006315, FEBRA2006346, FEBRA2006354, FEBRA2006357, FEBRA2006368, FEBRA2006370, FEBRA2006379, FEBRA2006396, FEBRA2006409, FEBRA2006418, FEBRA2006427, FEBRA2006476, FEBRA2006491, FEBRA2006519, FEBRA2006521, FEBRA2006553, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007551, FEBRA2007566, FEBRA2007589, FEBRA2007613, FEBRA2007620, FEBRA2007708, FEBRA2007714, FEBRA2007793, FEBRA2007818, FEBRA2007823, FEBRA2007901, FEBRA2007931, FEBRA2007957, FEBRA2008009, FEBRA2008036, FEBRA2008086, FEBRA2008134, FEBRA2008135, FEBRA2008159, FEBRA2008165, FEBRA2008172, FEBRA2008210, FEBRA2008255, FEBRA2008265, FEBRA2008266, FEBRA2008282, FEBRA2008287, FEBRA2008302, FEBRA2008311, FEBRA2008341, FEBRA2008360, FEBRA2008459, FEBRA2008468, FEBRA2008475, FEBRA2008481, FEBRA2008542, FEBRA2008583, FEBRA2008660, FEBRA2008662, FEBRA2008681, FEBRA2008741, FEBRA2008755, FEBRA2008836, FEBRA2008859, FEBRA2008861, FEBRA2008866, FEBRA2008881, FEBRA2008928, FEBRA2008983, FEBRA2009016, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009437, FEBRA2009478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013905, FEBRA2013951, FEBRA2014010, FEBRA2014122, FEBRA2014198, FEBRA2014213, FEBRA2014297, FEBRA2014417, FEBRA2014443, FEBRA2014578, FEBRA2014939, FEBRA2015076, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018433, FEBRA2018476, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022148, FEBRA2022204, FEBRA2022255, FEBRA2022322, FEBRA2022504, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023377, FEBRA2023498, FEBRA2023550, FEBRA2023721, FEBRA2023927, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025463, FEBRA2025477, FEBRA2025838, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEHRT2000325, FEHRT2000364, FEKID1000018, FELIV1000101, FELIV1000137, FELIV1000164, FELNG2000241, HCASM1000021, HCASM1000050, HCASM1000061, HCASM1000130, HCASM2000138, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000015, HCHON1000131, HCHON1000142, HCHON1000166, HCHON1000176, HCHON1000191, HCHON2000038, HCHON2000062, HCHON2000100, HCHON2000156, HCHON2000199, HCHON2000212, HCHON2000244, HCHON2000265, HCHON2000271, HCHON2000315, HCHON2000322, HCHON2000323, HCHON2000344, HCHON2000373, HCHON2000395, HCHON2000417, HCHON2000418, HCHON2000503, HCHON2000660, HCHON2000663, HCHON2000673, HCHON2000676, HCHON2000698, HCHON2000699, HCHON2000705, HCHON2000743, HCHON2000815, HCHON2000817, HCHON2000818, HCHON2000819, HCHON2000832, HCHON2000851, HCHON2000898, HCHON2000935, HCHON2000942, HCHON2000956, HCHON2000994, HCHON2001084, HCHON2001099, HCHON2001108, HCHON2001110, HCHON2001116, HCHON2001161, HCHON2001214, HCHON2001233, HCHON2001269, HCHON2001434, HCHON2001450, HCHON2001453, HCHON2001497, HCHON2001505, HCHON2001519, HCHON2001523, HCHON2001546, HCHON2001548, HCHON2001577, HCHON2001604, HCHON2001607, HCHON2001646, HCHON2001665, HCHON2001712, HCHON2001768, HCHON2001801, HCHON2001853, HCHON2002123, HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005921, HCHON2005987, HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HCHON2010543, HEART1000010, HEART1000074, HEART1000088, HEART1000099, HEART1000102, HEART1000118, HEART1000132, HEART1000139, HEART1000142, HEART1000149, HEART1000151, HEART1000173, HEART1000185, HEART2000024, HEART2000035, HEART2000099, HEART2000298, HEART2000306, HEART2000309, HEART2000411, HEART2000418, HEART2000444, HEART2000448, HEART2000492, HEART2000506, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006521, HEART2006557, HEART2006787, HEART2006789, HEART2006909, HEART2007031, HEART2007231, HEART2007443, HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC1000083, HHDPC1000114, HHDPC1000163, HHDPC2000055, HHDPC2000102, HHDPC2000104, HHDPC2000115, HHDPC2000258, HHDPC2000315, HHDPC2000455, HHDPC2000462, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006862, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030, HLUNG1000034, HLUNG1000037, HLUNG1000053, HLUNG1000064, HLUNG1000084, HLUNG1000091, HLUNG1000104, HLUNG1000105, HLUNG1000110, HLUNG1000151, HLUNG1000169, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363, HLUNG2000431, HLUNG2000480, HLUNG2000489, HLUNG2000501, HLUNG2000549, HLUNG2000557, HLUNG2000570, HLUNG2000751, HLUNG2000761, HLUNG2000777, HLUNG2000846, HLUNG2000870, HLUNG2000884, HLUNG2000926, HLUNG2000950, HLUNG2000955, HLUNG2000970, HLUNG2001013, HLUNG2001126, HLUNG2001144, HLUNG2001214, HLUNG2001439, HLUNG2001459, HLUNG2001507, HLUNG2001518, HLUNG2001633, HLUNG2001677, HLUNG2001712, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002465, HLUNG2002562, HLUNG2002648, HLUNG2002659, HLUNG2002707, HLUNG2002916, HLUNG2002942, HLUNG2002949, HLUNG2002958, HLUNG2002982, HLUNG2003003, HLUNG2003042, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004067, HLUNG2004154, HLUNG2004170, HLUNG2004217, HLUNG2004273, HLUNG2004388, HLUNG2004416, HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006812, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008139, HLUNG2008153, HLUNG2008225, HLUNG2008235, HLUNG2008247, HLUNG2008333, HLUNG2008348, HLUNG2008384, HLUNG2008396, HLUNG2008439, HLUNG2008480, HLUNG2008521, HLUNG2008833, HLUNG2008875, HLUNG2008885, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000137, HSYRA1000148, HSYRA1000178, HSYRA2000044, HSYRA2000137, HSYRA2000190, HSYRA2000221, HSYRA2000224, HSYRA2000232, HSYRA2000237, HSYRA2000253, HSYRA2000332, HSYRA2000371, HSYRA2000424, HSYRA2000507, HSYRA2000510, HSYRA2000605, HSYRA2000629, HSYRA2000640, HSYRA2000706, HSYRA2000760, HSYRA2000787, HSYRA2000792, HSYRA2000828, HSYRA2000832, HSYRA2000925, HSYRA2000927, HSYRA2001003, HSYRA2001103, HSYRA2001105, HSYRA2001142, HSYRA2001153, HSYRA2001232, HSYRA2001255, HSYRA2001332, HSYRA2001353, HSYRA2001396, HSYRA2001420, HSYRA2001484, HSYRA2001552, HSYRA2001580, HSYRA2001615, HSYRA2001638, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007667, HSYRA2008154, HSYRA2008714, HSYRA2009102, IMR321000155, IMR321000158, IMR321000161, IMR321000165, IMR321000207, IMR321000210, IMR321000230, IMR321000242, IMR321000266, IMR322000010, IMR322000072, IMR322000107, IMR322000121, IMR322000127, IMR322000144, IMR322000152, IMR322000164, IMR322000178, IMR322000223, IMR322000243, IMR322000302, IMR322000316, IMR322000374, IMR322000379, IMR322000383, IMR322000418, IMR322000555, IMR322000604, IMR322000609, IMR322000656, IMR322000672, IMR322000742, IMR322000775, IMR322000791, IMR322000811, IMR322000819, IMR322000838, IMR322000859, IMR322000863, IMR322000917, IMR322000918, IMR322000919, IMR322000953, IMR322000973, IMR322000984, IMR322001018, IMR322001167, IMR322001185, IMR322001245, IMR322001317, IMR322001318, IMR322001322, IMR322001380, IMR322001438, IMR322001452, IMR322001491, IMR322001534, IMR322001541, IMR322001665, IMR322001670, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, IMR322019070, JCMLC2000273, KIDNE1000008, KIDNE1000012, KIDNE1000015, KIDNE1000028, KIDNE1000036, KIDNE1000064, KIDNE1000104, KIDNE1000121, KIDNE1000143, KIDNE1000145, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000287, KIDNE2000315, KIDNE2000317, KIDNE2000328, KIDNE2000330, KIDNE2000335, KIDNE2000349, KIDNE2000375, KIDNE2000383, KIDNE2000394, KIDNE2000422, KIDNE2000493, KIDNE2000497, KIDNE2000510, KIDNE2000513, KIDNE2000517, KIDNE2000519, KIDNE2000555, KIDNE2000624, KIDNE2000665, KIDNE2000678, KIDNE2000717, KIDNE2000721, KIDNE2000722, KIDNE2000801, KIDNE2000832, KIDNE2000833, KIDNE2000846, KIDNE2000867, KIDNE2000909, KIDNE2001117, KIDNE2001140, KIDNE2001162, KIDNE2001269, KIDNE2001361, KIDNE2001636, KIDNE2001713, KIDNE2001802, KIDNE2001840, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002262, KIDNE2002265, KIDNE2002438, KIDNE2002483, KIDNE2002725, KIDNE2002768, KIDNE2002795, KIDNE2002798, KIDNE2002839, KIDNE2002845, KIDNE2002872, KIDNE2002882, KIDNE2002883, KIDNE2002980, KIDNE2002991, KIDNE2003185, KIDNE2003188, KIDNE2003233, KIDNE2003305, KIDNE2003335, KIDNE2003357, KIDNE2003373, KIDNE2003377, KIDNE2003507, KIDNE2003752, KIDNE2003764, KIDNE2003837, KIDNE2003941, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004475, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006030, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006465, KIDNE2006580, KIDNE2006652, KIDNE2006687, KIDNE2006733, KIDNE2006760, KIDNE2006775, KIDNE2006811, KIDNE2006820, KIDNE2006880, KIDNE2007005, KIDNE2007040, KIDNE2007077, KIDNE2007186, KIDNE2007222, KIDNE2007352, KIDNE2007356, KIDNE2007422, KIDNE2007569, KIDNE2007810, KIDNE2007811, KIDNE2007944, KIDNE2007954, KIDNE2008022, KIDNE2008069, KIDNE2008072, KIDNE2008116, KIDNE2008117, KIDNE2008218, KIDNE2008315, KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012440, KIDNE2012453, KIDNE2012563, KIDNE2012601, KIDNE2012613, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327, KIDNE2016388, KIDNE2016464, KIDNE2016539, KIDNE2016570, KIDNE2016918, KIDNE2017007, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018352, KIDNE2018462, KIDNE2018493, KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000079, LIVER1000082, LIVER1000099, LIVER1000104, LIVER1000111, LIVER1000126, LIVER1000132, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000033, LIVER2000037, LIVER2000158, LIVER2000216, LIVER2000237, LIVER2000247, LIVER2000360, LIVER2000416, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2001608, LIVER2001835, LIVER2002638, LIVER2002644, LIVER2002660, LIVER2002702, LIVER2003008, LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006251, LIVER2006410, LIVER2006469, LIVER2006644, LIVER2006764, LIVER2007415, LIVER2007548, LIVER2007568, LIVER2007721, LIVER2007783, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000096, MAMGL1000100, MAMGL1000132, MAMGL1000151, MAMGL1000173, MAMGL1000178, MAMGL1000182, MAMGL1000184, MESAN1000032, MESAN1000035, MESAN1000050, MESAN1000079, MESAN1000101, MESAN1000121, MESAN1000147, MESAN1000180, MESAN2000023, MESAN2000026, MESAN2000067, MESAN2000167, MESAN2000264, MESAN2000291, MESAN2000434, MESAN2000457, MESAN2000462, MESAN2000501, MESAN2000608, MESAN2000620, MESAN2000711, MESAN2000894, MESAN2000909, MESAN2001450, MESAN2001627, MESAN2001770, MESAN2001979, MESAN2001980, MESAN2002113, MESAN2002122, MESAN2002147, MESAN2002186, MESAN2002424, MESAN2002519, MESAN2002687, MESAN2002709, MESAN2002724, MESAN2002844, MESAN2002978, MESAN2003035, MESAN2003037, MESAN2003039, MESAN2003058, MESAN2003101, MESAN2003190, MESAN2003322, MESAN2003444, MESAN2003490, MESAN2003529, MESAN2003622, MESAN2003646, MESAN2003662, MESAN2003709, MESAN2003851, MESAN2004138, MESAN2004288, MESAN2004575, MESAN2005284, MESAN2005303, MESAN2005371, MESAN2005689, MESAN2005724, MESAN2005808, MESAN2005811, MESAN2005957, MESAN2006022, MESAN2006043, MESAN2006328, MESAN2006580, MESAN2006599, MESAN2006743, MESAN2006953, MESAN2006971, MESAN2007032, MESAN2007440, MESAN2007597, MESAN2007755, MESAN2008222, MESAN2008334, MESAN2008415, MESAN2008460, MESAN2008536, MESAN2008679, MESAN2008729, MESAN2008821, MESAN2008926, MESAN2008936, MESAN2009081, MESAN2009105, MESAN2009311, MESAN2009418, MESAN2009503, MESAN2009522, MESAN2009580, MESAN2010031, MESAN2010114, MESAN2010312, MESAN2010321, MESAN2010664, MESAN2010681, MESAN2011545, MESAN2011597, MESAN2011632, MESAN2011977, MESAN2011984, MESAN2012054, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015708, MESAN2016159, MESAN2016304, MESAN2016409, MESAN2016552, MESAN2016965, MESAN2017133, MESAN2017152, MESAN2017373, MESAN2017417, MESAN2018209, MESAN2018576, MESAN2018670, MESAN2018699, MESTC1000042, MESTC1000147, MESTC2000150, MESTC2000153, MESTC2000170, N1ESE2000698, NB9N41000011, NB9N41000121, NB9N41000142, NB9N41000146, NB9N41000340, NB9N42000104, NB9N42000122, NB9N42000196, NB9N42000281, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062, NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000091, NOVAR1000102, NOVAR2000038, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000004, NT2NE1000014, NT2NE1000018, NT2NE1000023, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000195, NT2NE2000222, NT2NE2000259, NT2NE2000284, NT2NE2000299, NT2NE2000353, NT2NE2000356, NT2NE2000369, NT2NE2000374, NT2NE2000383, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000809, NT2NE2000864, NT2NE2000909, NT2NE2000950, NT2NE2000951, NT2NE2000980, NT2NE2001000, NT2NE2001005, NT2NE2001021, NT2NE2001040, NT2NE2001048, NT2NE2001049, NT2NE2001142, NT2NE2001156, NT2NE2001180, NT2NE2001181, NT2NE2001188, NT2NE2001191, NT2NE2001247, NT2NE2001252, NT2NE2001324, NT2NE2001326, NT2NE2001337, NT2NE2001364, NT2NE2001372, NT2NE2001403, NT2NE2001428, NT2NE2001432, NT2NE2001435, NT2NE2001442, NT2NE2001524, NT2NE2001530, NT2NE2001545, NT2NE2001598, NT2NE2001623, NT2NE2001626, NT2NE2001634, NT2NE2001648, NT2NE2001655, NT2NE2001660, NT2NE2001666, NT2NE2001677, NT2NE2001697, NT2NE2001698, NT2NE2001723, NT2NE2001725, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003185, NT2NE2003252, NT2NE2003280, NT2NE2003308, NT2NE2003309, NT2NE2003315, NT2NE2003393, NT2NE2003408, NT2NE2003457, NT2NE2003485, NT2NE2003540, NT2NE2003569, NT2NE2003571, NT2NE2003705, NT2NE2003752, NT2NE2003887, NT2NE2003921, NT2NE2004255, NT2NE2004378, NT2NE2004490, NT2NE2004519, NT2NE2004606, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005419, NT2NE2005441, NT2NE2005517, NT2NE2005537, NT2NE2005555, NT2NE2005588, NT2NE2005676, NT2NE2005784, NT2NE2005804, NT2NE2005821, NT2NE2005876, NT2NE2005890, NT2NE2005921, NT2NE2005968, NT2NE2006087, NT2NE2006103, NT2NE2006217, NT2NE2006288, NT2NE2006382, NT2NE2006387, NT2NE2006427, NT2NE2006432, NT2NE2006458, NT2NE2006478, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010842, NT2NE2010854, NT2NE2011033, NT2NE2011036, NT2NE2011107, NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014104, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018594, NT2NE2018739, NT2NE2018916, NT2NE2019056, NT2NE2019076, NT2NE2019091, NT2NE2019099, NT2RI1000016, NT2RI1000027, NT2RI1000035, NT2RI1000164, NT2RI1000172, NT2RI2000004, NT2RI2000007, NT2RI2000064, NT2RI2000107, NT2RI2000116, NT2RI2000117, NT2RI2000128, NT2RI2000133, NT2RI2000167, NT2RI2000173, NT2RI2000187, NT2RI2000270, NT2RI2000276, NT2RI2000282, NT2RI2000284, NT2RI2000294, NT2RI2000313, NT2RI2000341, NT2RI2000348, NT2RI2000412, NT2RI2000421, NT2RI2000480, NT2RI2000575, NT2RI2000588, NT2RI2000597, NT2RI2000669, NT2RI2000685, NT2RI2000688, NT2RI2000689, NT2RI2000704, NT2RI2000738, NT2RI2000775, NT2RI2000822, NT2RI2000829, NT2RI2000841, NT2RI2000865, NT2RI2000932, NT2RI2000974, NT2RI2000987, NT2RI2001010, NT2RI2001017, NT2RI2001030, NT2RI2001054, NT2RI2001083, NT2RI2001091, NT2RI2001167, NT2RI2001178, NT2RI2001230, NT2RI2001235, NT2RI2001244, NT2RI2001307, NT2RI2001385, NT2RI2001409, NT2RI2001410, NT2RI2001422, NT2RI2001425, NT2RI2001449, NT2RI2001450, NT2RI2001474, NT2RI2001519, NT2RI2001595, NT2RI2001621, NT2RI2001624, NT2RI2001726, NT2RI2001731, NT2RI2001769, NT2RI2001782, NT2RI2001836, NT2RI2001846, NT2RI2001866, NT2RI2001910, NT2RI2001952, NT2RI2002022, NT2RI2002041, NT2RI2002120, NT2RI2002152, NT2RI2002153, NT2RI2002179, NT2RI2002252, NT2RI2002260, NT2RI2002266, NT2RI2002270, NT2RI2002334, NT2RI2002359, NT2RI2002447, NT2RI2002481, NT2RI2002507, NT2RI2002517, NT2RI2002540, NT2RI2002549, NT2RI2002554, NT2RI2002564, NT2RI2002585, NT2RI2002592, NT2RI2002602, NT2RI2002699, NT2RI2002802, NT2RI2002819, NT2RI2002822, NT2RI2002847, NT2RI2002852, NT2RI2002923, NT2RI2002926, NT2RI2002958, NT2RI2002970, NT2RI2003011, NT2RI2003067, NT2RI2003184, NT2RI2003205, NT2RI2003222, NT2RI2003301, NT2RI2003317, NT2RI2003338, NT2RI2003344, NT2RI2003360, NT2RI2003361, NT2RI2003383, NT2RI2003392, NT2RI2003399, NT2RI2003419, NT2RI2003481, NT2RI2003487, NT2RI2003556, NT2RI2003655, NT2RI2003667, NT2RI2003678, NT2RI2003751, NT2RI2003884, NT2RI2003921, NT2RI2003973, NT2RI2003993, NT2RI2004059, NT2RI2004078, NT2RI2004079, NT2RI2004093, NT2RI2004136, NT2RI2004157, NT2RI2004188, NT2RI2004190, NT2RI2004209, NT2RI2004230, NT2RI2004284, NT2RI2004312, NT2RI2004381, NT2RI2004398, NT2RI2004410, NT2RI2004442, NT2RI2004468, NT2RI2004535, NT2RI2004606, NT2RI2004608, NT2RI2004618, NT2RI2004783, NT2RI2004818, NT2RI2004840, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005130, NT2RI2005150, NT2RI2005166, NT2RI2005239, NT2RI2005246, NT2RI2005368, NT2RI2005382, NT2RI2005405, NT2RI2005473, NT2RI2005520, NT2RI2005564, NT2RI2005579, NT2RI2005621, NT2RI2005628, NT2RI2005670, NT2RI2005710, NT2RI2005723, NT2RI2005772, NT2RI2005811, NT2RI2005814, NT2RI2005816, NT2RI2005818, NT2RI2005966, NT2RI2006070, NT2RI2006071, NT2RI2006072, NT2RI2006081, NT2RI2006126, NT2RI2006127, NT2RI2006183, NT2RI2006257, NT2RI2006271, NT2RI2006294, NT2RI2006345, NT2RI2006403, NT2RI2006412, NT2RI2006445, NT2RI2006487, NT2RI2006553, NT2RI2006565, NT2RI2006640, NT2RI2006659, NT2RI2006662, NT2RI2006667, NT2RI2006679, NT2RI2006682, NT2RI2006686, NT2RI2006703, NT2RI2006716, NT2RI2006762, NT2RI2006788, NT2RI2006838, NT2RI2006855, NT2RI2006943, NT2RI2006973, NT2RI2007046, NT2RI2007048, NT2RI2007054, NT2RI2007084, NT2RI2007096, NT2RI2007116, NT2RI2007148, NT2RI2007214, NT2RI2007277, NT2RI2007303, NT2RI2007334, NT2RI2007377, NT2RI2007386, NT2RI2007406, NT2RI2007439, NT2RI2007445, NT2RI2007468, NT2RI2007469, NT2RI2007507, NT2RI2007629, NT2RI2007723, NT2RI2007729, NT2RI2007751, NT2RI2007754, NT2RI2007827, NT2RI2007853, NT2RI2007877, NT2RI2007879, NT2RI2007884, NT2RI2007891, NT2RI2007956, NT2RI2007965, NT2RI2007987, NT2RI2008007, NT2RI2008045, NT2RI2008050, NT2RI2008171, NT2RI2008188, NT2RI2008204, NT2RI2008221, NT2RI2008233, NT2RI2008455, NT2RI2008502, NT2RI2008526, NT2RI2008566, NT2RI2008598, NT2RI2008622, NT2RI2008656, NT2RI2008791, NT2RI2008801, NT2RI2008808, NT2RI2008812, NT2RI2008841, NT2RI2008952, NT2RI2009005, NT2RI2009065, NT2RI2009066, NT2RI2009083, NT2RI2009101, NT2RI2009103, NT2RI2009144, NT2RI2009151, NT2RI2009194, NT2RI2009215, NT2RI2009259, NT2RI2009269, NT2RI2009281, NT2RI2009289, NT2RI2009301, NT2RI2009402, NT2RI2009517, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012659, NT2RI2012726, NT2RI2012990, NT2RI2013345, NT2RI2013357, NT2RI2013373, NT2RI2014247, NT2RI2014551, NT2RI2014683, NT2RI2014733, NT2RI2015239, NT2RI2015533, NT2RI2016128, NT2RI2016134, NT2RI2017529, NT2RI2018311, NT2RI2018363, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006340, NT2RI3006376, NT2RI3006666, NT2RI3006796, NT2RI3007065, NT2RI3007095, NT2RI3007158, NT2RI3007167, NT2RI3007213, NT2RI3007291, NT2RI3007443, NT2RI3007684, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000008, NT2RP6000032, NT2RP6000033, NT2RP6000039, NT2RP6000043, NT2RP6000059, NT2RP6000060, NT2RP6000072, NT2RP6000077, NT2RP6000078, NT2RP6000085, NT2RP6000086, NT2RP6000100, NT2RP6000110, NT2RP6000119, NT2RP6000127, NT2RP6000130, NT2RP6000140, NT2RP6000150, NT2RP700004.1, NT2RP7000069, NT2RP7000109, NT2RP7000112, NT2RP7000238, NT2RP7000259, NT2RP7000311, NT2RP7000359, NT2RP7000364, NT2RP7000380, NT2RP7000391, NT2RP7000425, NT2RP7000579, NT2RP7000586, NT2RP7000624, NT2RP7000812, NT2RP7000926, NT2RP7001074, NT2RP7001080, NT2RP7001156, NT2RP7001231, NT2RP7001306, NT2RP7001319, NT2RP7001335, NT2RP7001364, NT2RP7001532, NT2RP7001602, NT2RP7001856, NT2RP7001962, NT2RP7002028, NT2RP7002064, NT2RP7002151, NT2RP7002163, NT2RP7002243, NT2RP7002282, NT2RP7002478, NT2RP7002554, NT2RP7002603, NT2RP7002650, NT2RP7002738, NT2RP7002779, NT2RP7002829, NT2RP7002841, NT2RP7002875, NT2RP7002906, NT2RP7002978, NT2RP7003025, NT2RP7003050, NT2RP7003055, NT2RP7003084, NT2RP7003091, NT2RP7003107, NT2RP7003134, NT2RP7003148, NT2RP7003203, NT2RP7003439, NT2RP7003511, NT2RP7003629, NT2RP7003680, NT2RP7003724, NT2RP7004027, NT2RP7004080, NT2RP7004114, NT2RP7004204, NT2RP7004233, NT2RP7004260, NT2RP7004358, NT2RP7004396, NT2RP7004428, NT2RP7004481, NT2RP7004541, NT2RP7004641, NT2RP7004656, NT2RP7004687, NT2RP7004722, NT2RP7004728, NT2RP7004766, NT2RP7004790, NT2RP7004884, NT2RP7004915, NT2RP7004961, NT2RP7004964, NT2RP7004975, NT2RP7005118, NT2RP7005205, NT2RP7005219, NT2RP7005323, NT2RP7005468, NT2RP7005493, NT2RP7005513, NT2RP7005529, NT2RP7005629, NT2RP7005631, NT2RP7005669, NT2RP7005675, NT2RP7005846, NT2RP7006033, NT2RP7006160, NT2RP7006162, NT2RP7006223, NT2RP7006263, NT2RP7006296, NT2RP7006304, NT2RP7006374, NT2RP7006395, NT2RP7006408, NT2RP7006457, NT2RP7006539, NT2RP7006547, NT2RP7006601, NT2RP7006619, NT2RP7006621, NT2RP7006636, NT2RP7006853, NT2RP7006886, NT2RP7006887, NT2RP7006980, NT2RP7006986, NT2RP7006995, NT2RP7007114, NT2RP7007221, NT2RP7007226, NT2RP7007252, NT2RP7007269, NT2RP7007310, NT2RP7007359, NT2RP7007381, NT2RP7007387, NT2RP7007406, NT2RP7007480, NT2RP7007504, NT2RP7007530, NT2RP7007537, NT2RP7007580, NT2RP7007594, NT2RP7007617, NT2RP7007643, NT2RP7007766, NT2RP7007925, NT2RP7007930, NT2RP7008013, NT2RP7008015, NT2RP7008133, NT2RP7008137, NT2RP7008142, NT2RP7008144, NT2RP7008315, NT2RP7008360, NT2RP7008396, NT2RP7008406, NT2RP7008435, NT2RP7008441, NT2RP7008454, NT2RP7008544, NT2RP7008550, NT2RP7008557, NT2RP7008623, NT2RP7008657, NT2RP7008714, NT2RP7008720, NT2RP7008855, NT2RP7008863, NT2RP7009019, NT2RP7009030, NT2RP7009097, NT2RP7009168, NT2RP7009215, NT2RP7009225, NT2RP7009236, NT2RP7009253, NT2RP7009267, NT2RP7009322, NT2RP7009363, NT2RP7009370, NT2RP7009394, NT2RP7009397, NT2RP7009429, NT2RP7009439, NT2RP7009466, NT2RP7009481, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014178, NT2RP7014348, NT2RP7014420, NT2RP7014583, NT2RP7014721, NT2RP7014743, NT2RP7014778, NT2RP7014906, NT2RP7014910, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, -NT2RP7019835, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000047, NTONG1000052, NTONG1000075, NTONG1000087, NTONG1000123, NTONG1000130, NTONG1000161, NTONG1000172, NTONG1000182, NTONG1000213, NTONG1000214, NTONG1000246, NTONG1000247, NTONG2000040, NTONG2000107, NTONG2000231, NTONG2000334, NTONG2000363, NTONG2000413, NTONG2000492, NTONG2000499, NTONG2000531, NTONG2000583, NTONG2000800, NTONG2000858, NTONG2000876, NTONG2000878, NTONG2000966, NTONG2000985, NTONG2001014, NTONG2001079, NTONG2001157, NTONG2001173, NTONG2001220, NTONG2001362, NTONG2001428, NTONG2001532, NTONG2001612, NTONG2001619, NTONG2001642, NTONG2001657, NTONG2001676, NTONG2001762, NTONG2002278, NTONG2002582, NTONG2002807, NTONG2002948, NTONG2002970, NTONG2002985, NTONG2003158, NTONG2003210, NTONG2003316, NTONG2003409, NTONG2003454, NTONG2003515, NTONG2003805, NTONG2003839, NTONG2003852, NTONG2003897, NTONG2003928, NTONG2004095, NTONG2004308, NTONG2004521, NTONG2004614, NTONG2004690, NTONG2004806, NTONG2004829, NTONG2004844, NTONG2004991, NTONG2005062, NTONG2005086, NTONG2005137, NTONG2005153, NTONG2005265, NTONG2005277, NTONG2005363, NTONG2005373, NTONG2005391, NTONG2005480, NTONG2005577, NTONG2005657, NTONG2005801, NTONG2005822, NTONG2005897, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030, OCBBF1000042, OCBBF1000049, OCBBF1000054, OCBBF1000067, OCBBF1000080, OCBBF1000085, OCBBF1000091, OCBBF1000104, OCBBF1000114, OCBBF1000118, OCBBF1000119, OCBBF1000122, OCBBF1000130, OCBBF1000138, OCBBF1000145, OCBBF1000153, OCBBF1000175, OCBBF1000185, OCBBF1000192, OCBBF1000254, OCBBF2000013, OCBBF2000126, OCBBF2000178, OCBBF2000231, OCBBF2000277, OCBBF2000287, OCBBF2000323, OCBBF2000452, OCBBF2000467, OCBBF2000703, OCBBF2000712, OCBBF2000719, OCBBF2000824, OCBBF2000831, OCBBF2000904, OCBBF2000982, OCBBF2000998, OCBBF2001075, OCBBF2001101, OCBBF2001124, OCBBF2001166, OCBBF2001176, OCBBF2001186, OCBBF2001196, OCBBF2001210, OCBBF2001252, OCBBF2001307, OCBBF2001323, OCBBF2001389, OCBBF2001402, OCBBF2001408, OCBBF2001416, OCBBF2001473, OCBBF2001494, OCBBF2001527, OCBBF2001528, OCBBF2001586, OCBBF2001639, OCBBF2001681, OCBBF2001706, OCBBF2001749, OCBBF2001794, OCBBF2001910, OCBBF2001931, OCBBF2001961, OCBBF2001983, OCBBF2002015, OCBBF2002083, OCBBF2002124, OCBBF2002161, OCBBF2002290, OCBBF2002357, OCBBF2002376, OCBBF2002615, OCBBF2002626, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003518, OCBBF2003543, OCBBF2003593, OCBBF2003744, OCBBF2003819, OCBBF2003925, OCBBF2004038, OCBBF2004104, OCBBF2004147, OCBBF2004168, OCBBF2004235, OCBBF2004259, OCBBF2004273, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843, OCBBF2005901, OCBBF2006005, OCBBF2006030, OCBBF2006058, OCBBF2006113, OCBBF2006154, OCBBF2006172, OCBBF2006214, OCBBF2006241, OCBBF2006313, OCBBF2006388, OCBBF2006438, OCBBF2006567, OCBBF2006624, OCBBF2006639, OCBBF2006660, OCBBF2006764, OCBBF2006849, OCBBF2006859, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007414, OCBBF2007415, OCBBF2007428, OCBBF2007478, OCBBF2007485, OCBBF2007520, OCBBF2007555, OCBBF2007610, OCBBF2007622, OCBBF2007756, OCBBF2007762, OCBBF2007931, OCBBF2007946, OCBBF2008005, OCBBF2008041, OCBBF2008116, OCBBF2008138, OCBBF2008144, OCBBF2008283, OCBBF2008330, OCBBF2008340, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770, OCBBF2008775, OCBBF2008790, OCBBF2008814, OCBBF2008822, OCBBF2009095, OCBBF2009115, OCBBF2009301, OCBBF2009352, OCBBF2009391, OCBBF2009536, OCBBF2009571, OCBBF2009583, OCBBF2009603, OCBBF2009772, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010841, OCBBF2010843, OCBBF2010863, OCBBF2010871, OCBBF2010931, OCBBF2010945, OCBBF2010978, OCBBF2010989, OCBBF2011021, OCBBF2011137, OCBBF2011160, OCBBF2011177, OCBBF2011228, OCBBF2011232, OCBBF2011283, OCBBF2011311, OCBBF2011536, OCBBF2011625, OCBBF2011669, OCBBF2011685, OCBBF2011706, OCBBF2011722, OCBBF2011767, OCBBF2011855, OCBBF2011872, OCBBF2011887, OCBBF2011897, OCBBF2011914, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012553, OCBBF2012678, OCBBF2012704, OCBBF2012714, OCBBF2012755, OCBBF2012812, OCBBF2012821, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789, OCBBF2013883, OCBBF2013926, OCBBF2014052, OCBBF2014064, OCBBF2014089, OCBBF2014292, OCBBF2014322, OCBBF2014386, OCBBF2014541, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016841, OCBBF2016928, OCBBF2017035, OCBBF2017055, OCBBF2017069, OCBBF2017306, OCBBF2017325, OCBBF2017398, OCBBF2017458, OCBBF2017489, OCBBF2017516, OCBBF2017665, OCBBF2017754, OCBBF2017815, OCBBF2017882, OCBBF2017888, OCBBF2017893, OCBBF2017920, OCBBF2018014, OCBBF2018084, OCBBF2018167, OCBBF2018206, OCBBF2018229, OCBBF2018234, OCBBF2018380, OCBBF2018563, OCBBF2018581, OCBBF2018618, OCBBF2018663, OCBBF2018687, OCBBF2018707, OCBBF2018828, OCBBF2018934, OCBBF2018956, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035226, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000323, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000180, PEBLM2000030, PEBLM2000112, PEBLM2000126, PEBLM2000147, PEBLM2000170, PEBLM2000180, PEBLM2000213, PEBLM2000248, PEBLM2000267, PEBLM2000270, PEBLM2000308, PEBLM2000321, PEBLM2000326, PEBLM2000333, PEBLM2000338, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004497, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006366, PEBLM2006709, PEBLM2006912, PEBLM2007112, PEBLM2007140, PEBLM2007296, PEBLM2007437, PEBLM2007447, PEBLM2007598, PEBLM2007832, PEBLM2007834, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000037, PLACE5000058, PLACE5000066, PLACE5000067, PLACE5000068, PLACE5000070, PLACE5000080, PLACE5000105, PLACE5000113, PLACE5000115, PLACE5000116, PLACE5000129, PLACE5000139, PLACE5000153, PLACE5000170, PLACE5000171, PLACE5000172, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000263, PLACE6000296, PLACE6000314, PLACE6000325, PLACE6000348, PLACE6000371, PLACE6000424, PLACE6000426, PLACE6000429, PLACE6000450, PLACE6000463, PLACE6000523, PLACE6000550, PLACE6000552, PLACE6000555, PLACE6000619, PLACE6000630, PLACE6000953, PLACE6001118, PLACE6001153, PLACE6001185, PLACE6001262, PLACE6001294, PLACE6001443, PLACE6001536, PLACE6001557, PLACE6001712, PLACE6001740, PLACE6001823, PLACE6001886, PLACE6001923, PLACE6001925, PLACE6001933, PLACE6002011, PLACE6002016, PLACE6002056, PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6002960, PLACE6003004, PLACE6003038, PLACE6003077, PLACE6003094, PLACE6003109, PLACE6003204, PLACE6003218, PLACE6003372, PLACE6003399, PLACE6003539, PLACE6003621, PLACE6003705, PLACE6003740, PLACE6003771, PLACE6003796, PLACE6003839, PLACE6003850, PLACE6004005, PLACE6004219, PLACE6004336, PLACE6004368, PLACE6004380, PLACE6004396, PLACE6004397, PLACE6004454, PLACE6004464, PLACE6004491, PLACE6004578, PLACE6004663, PLACE6004687, PLACE6004738, PLACE6004931, PLACE6004966, PLACE6004993, PLACE6005007, PLACE6005029, PLACE6005108, PLACE6005231, PLACE6005234, PLACE6005283, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006042, PLACE6006077, PLACE6006137, PLACE6006158, PLACE6006186, PLACE6006217, PLACE6006222, PLACE6006266, PLACE6006287, PLACE6006394, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008775, PLACE6008793, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010484, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011057, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012574, PLACE6012908, PLACE6012936, PLACE6012942, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016254, PLACE6016383, PLACE6016942, PLACE6016984, PLACE6017002, PLACE6017431, PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018497, PLACE6018769, PLACE6018834, PLACE6018843, PLACE6018863, PLACE6018938, PLACE6019195, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000039, PROST1000065, PROST1000083, PROST1000085, PROST1000110, PROST1000120, PROST1000123, PROST1000136, PROST1000144, PROST1000145, PROST1000152, PROST1000167, PROST1000184, PROST1000215, PROST1000217, PROST1000246, PROST1000262, PROST1000298, PROST1000322, PROST1000334, PROST1000362, PROST1000377, PROST1000383, PROST1000412, PROST1000419, PROST1000451, PROST1000480, PROST1000485, PROST1000493, PROST1000512, PROST1000527, PROST1000528, PROST1000536, PROST1000559, PROST1000564, PROST1000575, PROST2000036, PROST2000053, PROST2000067, PROST2000079, PROST2000105, PROST2000143, PROST2000162, PROST2000203, PROST2000206, PROST2000212, PROST2000241, PROST2000267, PROST2000273, PROST2000274, PROST2000277, PROST2000325, PROST2000337, PROST2000432, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002, PROST2001116, PROST2001138, PROST2001180, PROST2001213, PROST2001283, PROST2001414, PROST2001415, PROST2001465, PROST2001540, PROST2001552, PROST2001599, PROST2001611, PROST2001636, PROST2001739, PROST2001796, PROST2001805, PROST2001823, PROST2001883, PROST2001899, PROST2001997, PROST2001998, PROST2002078, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003302, PROST2003303, PROST2003324, PROST2003338, PROST2003340, PROST2003396, PROST2003428, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004258, PROST2004332, PROST2004481, PROST2004526, PROST2004570, PROST2004727, PROST2004739, PROST2004742, PROST2004744, PROST2004817, PROST2005067, PROST2005076, PROST2005093, PROST2005121, PROST2005131, PROST2005143, PROST2005228, PROST2005272, PROST2005285, PROST2005426, PROST2005466, PROST2005630, PROST2005683, PROST2005788, PROST2005793, PROST2005880, PROST2005886, PROST2005904, PROST2005919, PROST2005943, PROST2006008, PROST2006020, PROST2006030, PROST2006196, PROST2006201, PROST2006282, PROST2006343, PROST2006450, PROST2006491, PROST2006510, PROST2006536, PROST2006579, PROST2006688, PROST2006737, PROST2006766, PROST2006782, PROST2006933, PROST2006988, PROST2007122, PROST2007200, PROST2007237, PROST2007248, PROST2007289, PROST2007317, PROST2007328, PROST2007382, PROST2007389, PROST2007416, PROST2007528, PROST2007818, PROST2007871, PROST2007950, PROST2007956, PROST2007974, PROST2007988, PROST2008079, PROST2008088, PROST2008243, PROST2008245, PROST2008268, PROST2008447, PROST2008468, PROST2008472, PROST2008516, PROST2008724, PROST2008770, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010579, PROST2010606, PROST2010885, PROST2011012, PROST2011038, PROST2011105, PROST2011297, PROST2011439, PROST2011482, PROST2011631, PROST2011660, PROST2012005, PROST2012007, PROST2012016, PROST2012088, PROST2012157, PROST2012190, PROST2012249, PROST2012353, PROST2012400, PROST2012448, PROST2012504, PROST2012542, PROST2012550, PROST2012740, PROST2012745, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015137, PROST2015198, PROST2015243, PROST2015246, PROST2015251, PROST2015287, PROST2015332, PROST2015457, PROST2015464, PROST2015537, PROST2015545, PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2016980, PROST2017098, PROST2017128, PROST2017203, PROST2017367, PROST2017413, PROST2017441, PROST2017578, PROST2017612, PROST2017617, PROST2017700, PROST2017701, PROST2017729, PROST2017836, PROST2017910, PROST2017972, PROST2018030, PROST2018235, PROST2018437, PROST2018487, PROST2018511, PROST2018583, PROST2018588, PROST2018607, PROST2018788, PROST2018902, PROST2018922, PROST2018977, PROST2019164, PROST2019253, PROST2019296, PROST2019398, PROST2019487, PROST2019781, PUAEN1000039, PUAEN1000057, PUAEN1000085, PUAEN1000087, PUAEN1000121, PUAEN1000147, PUAEN1000161, PUAEN1000175, PUAEN1000239, PUAEN1000275, PUAEN1000276, PUAEN1000288, PUAEN1000322, PUAEN2000080, PUAEN2000152, PUAEN2000175, PUAEN2000247, PUAEN2000394, PUAEN2000497, PUAEN2000594, PUAEN2000684, PUAEN2000942, PUAEN2001260, PUAEN2001345, PUAEN2001526, PUAEN2001586, PUAEN2001882, PUAEN2002473, PUAEN2002489, PUAEN2002552, PUAEN2002568, PUAEN2002616, PUAEN2002758, PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007044, PUAEN2007350, PUAEN2007785, PUAEN2007898, PUAEN2008123, PUAEN2008228, PUAEN2008314, PUAEN2008515, PUAEN2008815, PUAEN2008939, PUAEN2008980, PUAEN2008985, PUAEN2009534, PUAEN2009655, PUAEN2009795, PUAEN2009852, PUAEN2010346, PUAEN2010824, RECTM1000051, RECTM1000141, RECTM2000220, RECTM2000349, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000047, SALGL1000065, SALGL1000107, SALGL1000157, SALGL1000171, SKMUS1000014, SKMUS1000030, SKMUS1000064, SKMUS1000067, SKMUS1000084, SKMUS1000102, SKMUS1000104, SKMUS1000110, SKMUS1000118, SKMUS1000124, SKMUS1000129, SKMUS2000020, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000365, SKMUS2000380, SKMUS2000390, SKMUS2000416, SKMUS2000458, SKMUS2000467, SKMUS2000468, SKMUS2000484, SKMUS2000515, SKMUS2000679, SKMUS2000690, SKMUS2000701, SKMUS2000705, SKMUS2000710, SKMUS2000724, SKMUS2000726, SKMUS2000757, SKMUS2000774, SKMUS2000780, SKMUS2000847, SKMUS2000863, SKMUS2000873, SKMUS2000894, SKMUS2000898, SKMUS2000902, SKMUS2000931, SKMUS2000933, SKMUS2000945, SKMUS2000961, SKMUS2000973, SKMUS2000980, SKMUS2001008, SKMUS2001014, SKMUS2001129, SKMUS2001147, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001501, SKMUS2001543, SKMUS2001580, SKMUS2001585, SKMUS2001608, SKMUS2001631, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000110, SKNMC1000123, SKNMC1000124, SKNMC1000137, SKNMC1000159, SKNMC1000264, SKNMC2000224, SKNMC2000256, SKNMC2000356, SKNMC2000374, SKNMC2000583, SKNMC2000593, SKNMC2000612, SKNMC2000622, SKNMC2000966, SKNMC2001113, SKNMC2001324, SKNMC2001503, SKNMC2001555, SKNMC2001596, SKNMC2001623, SKNMC2002402, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000002, SKNSH1000086, SKNSH1000101, SKNSH1000174, SKNSH1000301, SKNSH1000308, SKNSH1000416, SKNSH2000051, SKNSH2000101, SKNSH2000151, SKNSH2000163, SKNSH2000250, SKNSH2000319, SKNSH2000482, SKNSH2000516, SKNSH2000550, SKNSH2000971, SKNSH2001222, SKNSH2001875, SKNSH2001931, SKNSH2002054, SKNSH2002768, SKNSH2002866, SKNSH2003064, SKNSH2003174, SKNSH2003422, SKNSH2003466, SKNSH2003483, SKNSH2003528, SKNSH2003633, SKNSH2004039, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009357, SKNSH2009435, SKNSH2009991, SKNSH2010015, SMINT1000012, SMINT1000016, SMINT1000039, SMINT1000042, SMINT1000048, SMINT1000051, SMINT1000054, SMINT1000057, SMINT1000071, SMINT1000075, SMINT1000078, SMINT1000091, SMINT1000100, SMINT1000103, SMINT1000117, SMINT1000118, SMINT1000137, SMINT1000192, SMINT2000007, SMINT2000032, SMINT2000040, SMINT2000060, SMINT2000073, SMINT2000084, SMINT2000145, SMINT2000159, SMINT2000176, SMINT2000185, SMINT2000227, SMINT2000229, SMINT2000232, SMINT2000239, SMINT2000267, SMINT2000277, SMINT2000396, SMINT2000400, SMINT2000454, SMINT2000518, SMINT2000529, SMINT2000530, SMINT2000537, SMINT2000538, SMINT2000541, SMINT2000545, SMINT2000558, SMINT2000562, SMINT2000568, SMINT2000602, SMINT2000609, SMINT2000629, SMINT2000659, SMINT2000694, SMINT2000747, SMINT2000811, SMINT2000824, SMINT2000933, SMINT2000984, SMINT2001114, SMINT2001158, SMINT2001183, SMINT2001199, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001818, SMINT2001950, SMINT2002126, SMINT2002159, SMINT2002202, SMINT2002210, SMINT2002224, SMINT2002281, SMINT2002311, SMINT2002314, SMINT2002414, SMINT2002457, SMINT2002620, SMINT2002689, SMINT2002743, SMINT2002778, SMINT2002880, SMINT2002882, SMINT2002884, SMINT2002899, SMINT2002974, SMINT2002976, SMINT2003003, SMINT2003128, SMINT2003150, SMINT2003169, SMINT2003317, SMINT2003340, SMINT2003386, SMINT2003423, SMINT2003505, SMINT2003569, SMINT2003641, SMINT2003644, SMINT2003866, SMINT2003905, SMINT2004023, SMINT2004037, SMINT2004047, SMINT2004086, SMINT2004144, SMINT2004280, SMINT2004299, SMINT2004339, SMINT2004350, SMINT2004414, SMINT2004473, SMINT2004547, SMINT2004583, SMINT2004589, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005368, SMINT2005387, SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008960, SMINT2009119, SMINT2009121, SMINT2009212, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010901, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, -SMINT2012040, SMINT2012122, SMINT2012179, SMINT2012195, SMINT2012285, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000106, SPLEN1000116, SPLEN1000134, SPLEN1000139, SPLEN1000143, SPLEN1000144, SPLEN1000145, SPLEN1000156, SPLEN1000163, SPLEN1000165, SPLEN1000178, SPLEN2000020, SPLEN2000047, SPLEN2000064, SPLEN2000098, SPLEN2000126, SPLEN2000134, SPLEN2000143, SPLEN2000189, SPLEN2000197, SPLEN2000205, SPLEN2000210, SPLEN2000222, SPLEN2000242, SPLEN2000243, SPLEN2000247, SPLEN2000255, SPLEN2000258, SPLEN2000267, SPLEN2000270, SPLEN2000307, SPLEN2000310, SPLEN2000341, SPLEN2000348, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695, SPLEN2000698, SPLEN2000839, SPLEN2000874, SPLEN2000882, SPLEN2001135, SPLEN2001157, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001689, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002335, SPLEN2002343, SPLEN2002354, SPLEN2002385, SPLEN2002419, SPLEN2002451, SPLEN2002462, SPLEN2002463, SPLEN2002477, SPLEN2002662, SPLEN2002695, SPLEN2002707, SPLEN2002744, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004124, SPLEN2004148, SPLEN2004181, SPLEN2004220, SPLEN2004343, SPLEN2004346, SPLEN2004368, SPLEN2004611, SPLEN2004662, SPLEN2004844, SPLEN2004880, SPLEN2004984, SPLEN2005009, SPLEN2005227, SPLEN2005272, SPLEN2005342, SPLEN2005416, SPLEN2005429, SPLEN2005450, SPLEN2005474, SPLEN2005560, SPLEN2005727, SPLEN2005783, SPLEN2005790, SPLEN2005806, SPLEN2005818, SPLEN2005838, SPLEN2005869, SPLEN2005927, SPLEN2005939, SPLEN2006122, SPLEN2006143, SPLEN2006232, SPLEN2006268, SPLEN2006283, SPLEN2006305, SPLEN2006325, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008496, SPLEN2008591, SPLEN2008786, SPLEN2009081, SPLEN2009088, SPLEN2009163, SPLEN2009315, SPLEN2009340, SPLEN2009512, SPLEN2009513, SPLEN2009541, SPLEN2009548, SPLEN2009555, SPLEN2009581, SPLEN2009733, SPLEN2009803, SPLEN2009884, SPLEN2009918, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2011018, SPLEN2011021, SPLEN2011025, SPLEN2011086, SPLEN2011139, SPLEN2011145, SPLEN2011248, SPLEN2011252, SPLEN2011419, SPLEN2011422, SPLEN2011672, SPLEN2011678, SPLEN2011737, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012524, SPLEN2012525, SPLEN2012606, SPLEN2012611, SPLEN2012619, SPLEN2012624, SPLEN2012725, SPLEN2012743, SPLEN2012756, SPLEN2012800, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014080, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015310, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016241, SPLEN2016250, SPLEN2016268, SPLEN2016304, SPLEN2016356, SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016773, SPLEN2016781, SPLEN2016840, SPLEN2016863, SPLEN2016922, SPLEN2016932, SPLEN2016972, SPLEN2016973, SPLEN2017043, SPLEN2017104, SPLEN2017121, SPLEN2017147, SPLEN2017187, SPLEN2017189, SPLEN2017212, SPLEN2017318, SPLEN2017351, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019102, SPLEN2019131, SPLEN2019257, SPLEN2019311, SPLEN2019323, SPLEN2019349, SPLEN2019375, SPLEN2019379, SPLEN2019405, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021194, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027113, SPLEN2027157, SPLEN2027395, SPLEN2027488, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2039311, SPLEN2039379, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042535, SPLEN2042598, SPLEN2042714, SPLEN2042920, STOMA1000013, STOMA1000047, STOMA1000052, STOMA1000074, STOMA1000091, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000148, STOMA2000168, STOMA2000183, STOMA2000188, STOMA2000257, STOMA2000279, STOMA2000289, STOMA2000383, STOMA2000386, STOMA2000395, STOMA2000396, STOMA2000478, STOMA2000529, STOMA2000539, STOMA2000567, STOMA2000640, STOMA2000678, STOMA2000686, STOMA2000888, STOMA2001025, STOMA2001389, STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049, SYNOV1000118, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001033, SYNOV2001035, SYNOV2001088, SYNOV2001111, SYNOV2001144, SYNOV2001251, SYNOV2001262, SYNOV2001300, SYNOV2001356, SYNOV2001374, SYNOV2001390, SYNOV2001451, SYNOV2001457, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2009172, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007711, SYNOV4008245, SYNOV4008336, SYNOV4009129, SYNOV4009139, SYNOV4009298, T1ESE2000116, T1ESE2000609, T1ESE2002665, TBAES2000004, TBAES2000059, TBAES2000116, TBAES2000255, TBAES2000315, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002451, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000142, TESTI1000157, TESTI1000163, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000172, TESTI2000177, TESTI2000179, TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082, TESTI2001120, TESTI2001134, TESTI2001141, TESTI2001153, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001829, TESTI2001852, TESTI2001862, TESTI2001869, TESTI2001879, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002149, TESTI2002216, TESTI2002245, TESTI2002251, TESTI2002256, TESTI2002264, TESTI2002282, TESTI2002293, TESTI2002294, TESTI2002326, TESTI2002351, TESTI2002361, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002707, TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003418, TESTI2003419, TESTI2003432, TESTI2003454, TESTI2003475, TESTI2003476, TESTI2003498, TESTI2003512, TESTI2003533, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004031, TESTI2004073, TESTI2004085, TESTI2004089, TESTI2004111, TESTI2004119, TESTI2004122, TESTI2004153, TESTI2004163, TESTI2004169, TESTI2004207, TESTI2004215, TESTI2004227, TESTI2004229, TESTI2004243, TESTI2004255, TESTI2004287, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994, TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005408, TESTI2005415, TESTI2005470, TESTI2005492, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005759, TESTI2005775, TESTI2005827, TESTI2005835, TESTI2005860, TESTI2005892, TESTI2005908, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006457, TESTI2006460, TESTI2006465, TESTI2006483, TESTI2006499, TESTI2006523, TESTI2006543, TESTI2006565, TESTI2006572, TESTI2006588, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006748, TESTI2006774, TESTI2006853, TESTI2006866, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007220, TESTI2007234, TESTI2007320, TESTI2007346, TESTI2007358, TESTI2007402, TESTI2007405, TESTI2007407, TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009412, TESTI2009447, TESTI2009462, TESTI2009477, TESTI2009497, TESTI2009502, TESTI2009511, TESTI2009544, TESTI2009545, TESTI2009551, TESTI2009585, TESTI2009588, TESTI2009683, TESTI2009727, TESTI2009739, TESTI2009785, TESTI2009812, TESTI2009835, TESTI2009853, TESTI2009935, TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012152, TESTI2012155, TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257, TESTI2013264, TESTI2013268, TESTI2013295, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988, TESTI2015042, TESTI2015092, TESTI2015105, TESTI2015180, TESTI2015213, TESTI2015249, TESTI2015327, TESTI2015331, TESTI2015335, TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018687, TESTI2018697, TESTI2018838, TESTI2018867, TESTI2018941, TESTI2018957, TESTI2018974, TESTI2018975, TESTI2018997, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021138, TESTI2021297, TESTI2021315, TESTI2021358, TESTI2021425, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025448, TESTI2025454, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033441, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002072, TESTI4002141, TESTI4002195, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013602, TESTI4013667, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038156, TESTI4038223, TESTI4038258, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043140, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000017, THYMU1000025, THYMU1000029, THYMU1000032, THYMU1000033, THYMU1000060, THYMU1000074, THYMU1000083, THYMU1000098, THYMU1000103, THYMU1000105, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000331, THYMU1000336, THYMU1000359, THYMU1000366, THYMU1000382, THYMU1000393, THYMU1000394, THYMU1000399, THYMU1000426, THYMU1000440, THYMU1000459, THYMU1000473, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000436, THYMU2000440, THYMU2000489, THYMU2000492, THYMU2000519, THYMU2000570, THYMU2000602, THYMU2000660, THYMU2000671, THYMU2000672, THYMU2000702, THYMU2000767, THYMU2000775, THYMU2000800, THYMU2000879, THYMU2000932, THYMU2000950, THYMU2000971, THYMU2000982, THYMU2000990, THYMU2001007, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001839, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002815, THYMU2002841, THYMU2002852, THYMU2002910, THYMU2002950, THYMU2002983, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003446, THYMU2003470, THYMU2003479, THYMU2003499, THYMU2003632, THYMU2003650, THYMU2003700, THYMU2003760, THYMU2003803, THYMU2003855, THYMU2003907, THYMU2003932, THYMU2004003, THYMU2004008, THYMU2004042, THYMU2004108, THYMU2004111, THYMU2004123, THYMU2004139, THYMU2004152, THYMU2004169, THYMU2004284, THYMU2004320, THYMU2004336, THYMU2004356, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006913, THYMU2006946, THYMU2006965, THYMU2007025, THYMU2007036, THYMU2007060, THYMU2007065, THYMU2007095, THYMU2007112, THYMU2007146, THYMU2007179, THYMU2007258, THYMU2007307, THYMU2007308, THYMU2007339, THYMU2007350, THYMU2007384, THYMU2007415, THYMU2007467, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007886, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010161, THYMU2010192, THYMU2010448, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012024, THYMU2012073, THYMU2012113, THYMU2012273, THYMU2012401, THYMU2012492, THYMU2012507, THYMU2012565, THYMU2012574, THYMU2012625, THYMU2012631, THYMU2012665, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013136, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015762, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031368, THYMU2031579, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2035735, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015759, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032798, THYMU3032867, THYMU3033402, THYMU3033626, THYMU3033630, THYMU3033649, THYMU3033754, THYMU3033759, THYMU3034078, THYMU3034099, THYMU3034453, THYMU3034616, THYMU3034671, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000066, TKIDN1000164, TKIDN1000171, TKIDN1000192, TKIDN2000127, TKIDN2000240, TKIDN2000246, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2002738, TKIDN2003044, TKIDN2003059, TKIDN2003062, TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005487, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000030, TRACH1000038, TRACH1000057, TRACH1000063, TRACH1000074, TRACH1000100, TRACH1000106, TRACH1000125, TRACH1000140, TRACH1000144, TRACH1000181, TRACH1000193, TRACH1000205, TRACH1000212, TRACH1000240, TRACH2000002, TRACH2000015, TRACH2000024, TRACH2000032, TRACH2000045, TRACH2000047, TRACH2000081, TRACH2000083, TRACH2000148, TRACH2000185, TRACH2000196, TRACH2000235, TRACH2000237, TRACH2000243, TRACH2000250, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000420, TRACH2000496, TRACH2000497, TRACH2000502, TRACH2000522, TRACH2000540, TRACH2000559, TRACH2000660, TRACH2000665, TRACH2000675, TRACH2000677, TRACH2000702, TRACH2000703, TRACH2000767, TRACH2000775, TRACH2000780, TRACH2000807, TRACH2000873, TRACH2000894, TRACH2000898, TRACH2000920, TRACH2000923, TRACH2000944, TRACH2000959, TRACH2000981, TRACH2000986, TRACH2001037, TRACH2001085, TRACH2001109, TRACH2001121, TRACH2001147, TRACH2001154, TRACH2001188, TRACH2001192, TRACH2001249, TRACH2001275, TRACH2001314, TRACH2001335, TRACH2001339, TRACH2001347, TRACH2001395, TRACH2001400, TRACH2001403, TRACH2001416, TRACH2001443, TRACH2001463, TRACH2001474, TRACH2001478, TRACH2001523, TRACH2001525, TRACH2001532, TRACH2001549, TRACH2001592, TRACH2001596, TRACH2001621, TRACH2001646, TRACH2001651, TRACH2001684, TRACH2001898, TRACH2001933, TRACH2001968, TRACH2001996, TRACH2002043, TRACH2002054, TRACH2002092, TRACH2002100, TRACH2002138, TRACH2002333, TRACH2002664, TRACH2002784, TRACH2002840, TRACH2002847, TRACH2002853, TRACH2002862, TRACH2002954, TRACH2002960, TRACH2002988, TRACH2003070, TRACH2003272, TRACH2003347, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005800, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006870, TRACH2006918, TRACH2007324, TRACH2007358, TRACH2007483, TRACH2007542, TRACH2007577, TRACH2007674, TRACH2007676, TRACH2007688, TRACH2007733, TRACH2007754, TRACH2007894, TRACH2007969, TRACH2007988, TRACH2008081, TRACH2008112, TRACH2008113, TRACH2008127, TRACH2008130, TRACH2008278, TRACH2008300, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011113, TRACH2011290, TRACH2011293, TRACH2011302, TRACH2011500, TRACH2011574, TRACH2011894, TRACH2011956, TRACH2012000, TRACH2012138, TRACH2012242, TRACH2012298, TRACH2012370, TRACH2012376, TRACH2012387, TRACH2012497, TRACH2012536, TRACH2012562, TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084, TRACH2018262, TRACH2018273, TRACH2018278, TRACH2018317, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002192, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003547, TRACH3003586, TRACH3003683, TRACH3003780, TRACH3003805, TRACH3003832, TRACH3003872, TRACH3004068, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006800, TRACH3006889, TRACH3006985, TRACH3007097, TRACH3007268, TRACH3007274, TRACH3007277, TRACH3007391, TRACH3007541, TRACH3007595, TRACH3007627, TRACH3007689, TRACH3007766, TRACH3007866, TRACH3007995, TRACH3008042, TRACH3008061, TRACH3008093, TRACH3008201, TRACH3008508, TRACH3008535, TRACH3008629, TRACH3008632, TRACH3008688, TRACH3008697, TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036942, TRACH3036997, TRACH3037063, TRACH3037067, TRACH3037267, TRACH3037288, TRACH3037505, TRACH3037573, TRACH3037696, TRACH3037897, TRACH3038086, TSTOM1000135, TSTOM1000186, TSTOM2000139, TSTOM2000235, TSTOM2000442, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN1000186, UMVEN2000046, UMVEN2000069, UMVEN2000121, UMVEN2000133, UMVEN2000354, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000182, UTERU1000183, UTERU1000187, UTERU1000192, UTERU1000249, UTERU1000337, UTERU1000339, UTERU1000384, UTERU2000047, UTERU2000074, UTERU2000099, UTERU2000154, UTERU2000197, UTERU2000218, UTERU2000238, UTERU2000243, UTERU2000260, UTERU2000329, UTERU2000332, UTERU2000338, UTERU2000349, UTERU2000377, UTERU2000393, UTERU2000418, UTERU2000465, UTERU2000485, UTERU2000517, UTERU2000524, UTERU2000537, UTERU2000539, UTERU2000541, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001747, UTERU2001876, UTERU2002001, UTERU2002011, UTERU2002176, UTERU2002198, UTERU2002294, UTERU2002332, UTERU2002410, UTERU2002473, UTERU2002547, UTERU2002662, UTERU2002693, UTERU2002733, UTERU2002736, UTERU2002737, UTERU2002826, UTERU2002841, UTERU2002964, UTERU2002993, UTERU2003035, UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004039, UTERU2004061, UTERU2004073, UTERU2004163, UTERU2004299, UTERU2004461, UTERU2004564, UTERU2004664, UTERU2004698, UTERU2004807, UTERU2004861, UTERU2004929, UTERU2005004, UTERU2005050, UTERU2005069, UTERU2005074, UTERU2005179, UTERU2005292, UTERU2005346, UTERU2005354, UTERU2005446, UTERU2005449, UTERU2005450, UTERU2005533, UTERU2005601, UTERU2005664, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547, UTERU2006568, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639, UTERU2007724, UTERU2007924, UTERU2007942, UTERU2008018, UTERU2008019, UTERU2008027, UTERU2008040, UTERU2008077, UTERU2008130, UTERU2008302, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008939, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009510, UTERU2009538, UTERU2009540, UTERU2009776, UTERU2009904, UTERU2009951, UTERU2009972, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011962, UTERU2011968, UTERU2012031, UTERU2012101, UTERU2012114, UTERU2012230, UTERU2012252, UTERU2012286, UTERU2012333, UTERU2012526, UTERU2012581, UTERU2012610, UTERU2012615, UTERU2012688, UTERU2012703, UTERU2012715, UTERU2012741, UTERU2012767, UTERU2012786, UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001, UTERU2014024, UTERU2014167, UTERU2014223, UTERU2014398, UTERU2014464, UTERU2014548, UTERU2014601, UTERU2014631, UTERU2014668, UTERU2014678, UTERU2014728, UTERU2014998, UTERU2015062, UTERU2015087, UTERU2015108, UTERU2015190, UTERU2015198, UTERU2015202, UTERU2015405, UTERU2015640, UTERU2015830, UTERU2015880, UTERU2016147, UTERU2016157, UTERU2016426, UTERU2016464, UTERU2016669, UTERU2016757, UTERU2016761, UTERU2016799, UTERU2016822, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019005, UTERU2019038, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000402, UTERU3000645, UTERU3000665, UTERU3000670, UTERU3000727, UTERU3000738, UTERU3000828, UTERU3000844, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the D30ST, D60ST, D90ST, DFNES, ERLTF, FCBBF, and FEBRA libraries were analyzed for expression frequency. The analysis results are described below. Each is a relative value, where the expression level over all tissues is taken as 100. The larger this value, the greater the level of expression.

TABLE 30

| Clone Name | D30ST | D60ST | D90ST | DFNES | ERLTF | FCBBF | FEBRA |
|---|---|---|---|---|---|---|---|
| 3NB691000173 | 0 | 0 | 0 | 4.654 | 0 | 0 | 0 |
| 3NB692000281 | 0 | 0 | 2.968 | 1.297 | 0 | 0 | 0 |
| 3NB692000330 | 0 | 0 | 0 | 0 | 0 | 0 | 2.199 |
| 3NB692000374 | 0 | 0 | 0 | 0 | 0 | 4.007 | 0 |
| 3NB692000484 | 0 | 0 | 0 | 0 | 0 | 0 | 25.471 |
| 3NB692000545 | 0 | 0 | 0 | 0 | 0 | 5.212 | 7.033 |
| 3NB692000912 | 0 | 0 | 0 | 2.769 | 0 | 0.868 | 1.171 |
| 3NB692001022 | 0 | 0 | 0 | 0 | 0 | 0 | 3.102 |
| 3NB692001334 | 0 | 0 | 0 | 0 | 0 | 0.992 | 0.669 |
| 3NB692006952 | 0 | 0 | 0 | 0 | 0 | 0 | 14.594 |
| ADIPS2000425 | 0 | 0 | 0 | 0 | 0 | 1.434 | 0 |
| ADRGL1000002 | 0 | 0 | 0 | 0 | 0 | 12.168 | 0 |
| ADRGL1000018 | 0 | 0 | 0 | 0 | 0 | 0 | 12.055 |
| ADRGL1000033 | 0 | 0 | 0 | 0 | 0 | 7.054 | 0 |
| ADRGL1000065 | 0 | 0 | 0 | 0 | 0 | 31.255 | 0 |
| ADRGL2000006 | 0 | 0 | 0 | 0 | 0 | 3.841 | 0 |
| ADRGL2000097 | 0 | 0 | 0 | 39.306 | 0 | 0 | 0 |
| ADRGL2002477 | 0 | 0 | 0 | 0 | 0 | 15.715 | 0 |
| ADRGL2004676 | 0 | 0 | 0 | 0 | 0 | 8.053 | 32.599 |
| ADRGL2009273 | 0 | 0 | 0 | 8.187 | 0 | 7.699 | 0 |
| ASTRO1000165 | 0 | 0 | 0 | 0 | 0 | 4.095 | 0 |
| ASTRO2000014 | 0 | 0 | 5.538 | 0 | 0 | 3.035 | 0 |
| ASTRO2000046 | 0 | 0 | 5.502 | 2.405 | 0 | 0 | 0 |
| ASTRO2000141 | 17.731 | 0 | 20.479 | 0 | 0 | 0 | 0 |
| ASTRO2001029 | 14.640 | 0 | 0 | 0 | 0 | 4.633 | 0 |
| ASTRO2001823 | 0 | 0 | 0 | 0 | 0 | 8.045 | 0 |
| ASTRO2002202 | 0 | 0 | 0 | 0 | 0 | 4.257 | 0 |
| ASTRO2003212 | 0 | 0 | 0 | 0 | 0 | 27.675 | 0 |
| ASTRO2003461 | 0 | 0 | 0 | 0 | 0 | 11.005 | 0 |
| ASTRO2004032 | 0 | 0 | 0 | 0 | 0 | 11.259 | 15.193 |
| ASTRO2004974 | 0 | 0 | 0 | 0 | 0 | 3.590 | 0 |
| ASTRO2005553 | 0 | 0 | 0 | 0 | 71.006 | 0 | 6.460 |
| ASTRO2005687 | 0 | 0 | 0 | 13.138 | 0 | 0 | 0 |
| ASTRO2006920 | 0 | 0 | 0 | 35.357 | 0 | 0 | 0 |
| ASTRO2007047 | 0 | 0 | 0 | 0 | 0 | 3.861 | 0 |
| ASTRO2007948 | 0 | 0 | 0 | 0 | 0 | 4.627 | 6.243 |
| ASTRO2008972 | 0 | 0 | 0 | 0 | 0 | 28.772 | 0 |
| ASTRO2010819 | 0 | 0 | 0 | 0 | 0 | 6.215 | 3.355 |
| ASTRO2011422 | 0 | 0 | 0 | 0 | 0 | 26.805 | 0 |
| ASTRO2012552 | 0 | 0 | 0 | 8.711 | 0 | 2.731 | 0 |
| ASTRO2013585 | 0 | 0 | 6.573 | 0 | 13.358 | 0.901 | 3.646 |
| ASTRO2014863 | 0 | 0 | 0 | 0 | 0 | 12.483 | 0 |
| ASTRO2015529 | 21.631 | 0 | 0 | 0 | 0 | 0 | 9.237 |
| ASTRO2016491 | 0 | 0 | 6.790 | 2.968 | 0 | 1.861 | 2.511 |
| BGGI12000054 | 0 | 0 | 0 | 0 | 0 | 0.435 | 1.761 |
| BGGI12001075 | 0 | 0 | 0 | 0 | 0 | 3.657 | 0 |
| BGGI12001097 | 0 | 0 | 0 | 0 | 0 | 2.076 | 0 |
| BGGI12001682 | 0 | 0 | 0 | 0 | 0 | 0 | 5.149 |
| BLADE2006412 | 0 | 0 | 0 | 0 | 0 | 22.434 | 0 |
| BLADE2008461 | 0 | 0 | 0 | 0 | 67.927 | 0 | 0 |
| BNGH41000098 | 0 | 0 | 0 | 0 | 0 | 4.683 | 0 |
| BNGH42000739 | 0 | 0 | 0 | 0 | 0 | 2.299 | 3.102 |
| BNGH42005968 | 0 | 0 | 0 | 0 | 0 | 4.243 | 0 |
| BNGH42006135 | 0 | 0 | 0 | 0 | 0 | 1.220 | 0 |
| BNGH42007460 | 0 | 0 | 0 | 0 | 0 | 1.862 | 0 |
| BNGH42007709 | 5.375 | 15.453 | 3.104 | 0 | 0 | 0.425 | 0 |
| BNGH42007788 | 0 | 0 | 0 | 0 | 0 | 5.687 | 0 |
| BNGH42008603 | 0 | 0 | 0 | 8.524 | 0 | 0 | 0 |
| BNGH42009025 | 0 | 0 | 0 | 8.216 | 0 | 0 | 3.475 |
| BRACE1000042 | 0 | 0 | 0 | 0 | 0 | 2.812 | 0 |
| BRACE1000087 | 0 | 0 | 0 | 0 | 0 | 54.219 | 0 |
| BRACE1000093 | 0 | 0 | 0 | 0 | 0 | 72.245 | 0 |
| BRACE1000115 | 0 | 0 | 0 | 0 | 0 | 0 | 2.333 |
| BRACE1000159 | 0 | 0 | 0 | 0 | 0 | 0 | 4.603 |
| BRACE1000169 | 0 | 0 | 0 | 0 | 0 | 0 | 50.538 |
| BRACE1000187 | 0 | 0 | 0 | 0 | 0 | 0 | 20.185 |
| BRACE1000239 | 0 | 0 | 0 | 0 | 0 | 36.607 | 0 |
| BRACE2000307 | 0 | 0 | 0 | 0 | 0 | 6.528 | 2.936 |
| BRACE2000750 | 0 | 0 | 0 | 0 | 0 | 38.022 | 0 |
| BRACE2000975 | 0 | 0 | 0 | 0 | 0 | 3.550 | 1.597 |
| BRACE2001117 | 0 | 0 | 0 | 16.209 | 0 | 0 | 0 |
| BRACE2001154 | 0 | 0 | 0 | 0 | 0 | 0 | 26.486 |
| BRACE2001497 | 0 | 0 | 0 | 0 | 0 | 3.443 | 0 |
| BRACE2001892 | 0 | 0 | 0 | 0 | 0 | 7.835 | 9.817 |
| BRACE2002050 | 0 | 0 | 0 | 0 | 0 | 5.357 | 0 |
| BRACE2002091 | 0 | 0 | 0 | 0 | 0 | 3.901 | 0 |
| BRACE2002441 | 0 | 0 | 0 | 6.637 | 0 | 2.081 | 0 |
| BRACE2002478 | 0 | 0 | 0 | 0 | 0 | 26.777 | 0 |
| BRACE2002682 | 0 | 0 | 0 | 0 | 0 | 0 | 47.730 |
| BRACE2002860 | 0 | 0 | 33.949 | 0 | 0 | 4.651 | 6.276 |
| BRACE2002861 | 0 | 0 | 0 | 0 | 0 | 4.794 | 0 |
| BRACE2003037 | 0 | 0 | 0 | 0 | 0 | 0 | 10.765 |
| BRACE2003110 | 0 | 0 | 0 | 0 | 0 | 24.886 | 0 |
| BRACE2003172 | 3.794 | 0 | 0 | 0 | 0 | 9.006 | 4.051 |
| BRACE2003217 | 0 | 0 | 0 | 0 | 0 | 0 | 10.745 |
| BRACE2003254 | 0 | 0 | 0 | 0 | 0 | 1.673 | 0 |
| BRACE2003527 | 0 | 0 | 34.759 | 0 | 0 | 9.524 | 0 |
| BRACE2003785 | 0 | 0 | 0 | 0 | 0 | 3.590 | 2.422 |
| BRACE2003827 | 0 | 0 | 0 | 0 | 0 | 0 | 4.368 |
| BRACE2003892 | 0 | 0 | 0 | 0 | 0 | 0 | 16.322 |
| BRACE2003904 | 0 | 0 | 0 | 0 | 0 | 4.789 | 0 |
| BRACE2003944 | 0 | 0 | 0 | 6.101 | 0 | 0 | 0 |
| BRACE2005087 | 0 | 0 | 0 | 0 | 0 | 3.833 | 2.586 |
| BRACE2005151 | 0 | 0 | 0 | 0 | 57.578 | 3.882 | 0 |
| BRACE2005231 | 0 | 0 | 0 | 0 | 0 | 0 | 7.959 |
| BRACE2005251 | 0 | 0 | 0 | 0 | 0 | 0 | 26.348 |
| BRACE2005681 | 0 | 0 | 0 | 0 | 0 | 3.689 | 0 |
| BRACE2005719 | 0 | 0 | 0 | 0 | 0 | 5.487 | 14.807 |
| BRACE2005937 | 0 | 0 | 0 | 0 | 0 | 22.318 | 0 |
| BRACE2006055 | 0 | 0 | 0 | 0 | 0 | 7.391 | 4.987 |
| BRACE2006072 | 0 | 0 | 0 | 0 | 0 | 36.414 | 0 |
| BRACE2006075 | 0 | 0 | 0 | 0 | 0 | 29.977 | 0 |
| BRACE2006258 | 0 | 0 | 0 | 0 | 0 | 1.513 | 14.294 |
| BRACE2006264 | 0 | 0 | 0 | 0 | 0 | 15.593 | 0 |
| BRACE2006319 | 0 | 0 | 0 | 0 | 0 | 6.477 | 17.479 |
| BRACE2006344 | 0 | 0 | 0 | 0 | 0 | 13.207 | 0 |
| BRACE2006354 | 0 | 0 | 0 | 0 | 0 | 0 | 15.512 |
| BRACE2006514 | 0 | 0 | 11.715 | 0 | 0 | 1.605 | 2.166 |
| BRACE2006743 | 0 | 0 | 0 | 0 | 0 | 0 | 22.168 |
| BRACE2006900 | 6.007 | 0 | 0 | 0 | 0 | 0 | 0 |
| BRACE2006937 | 0 | 0 | 0 | 0 | 0 | 9.890 | 13.345 |
| BRACE2007064 | 0 | 0 | 0 | 0 | 0 | 6.004 | 12.152 |
| BRACE2007089 | 0 | 0 | 0 | 0 | 0 | 1.564 | 0 |
| BRACE2007109 | 0 | 0 | 0 | 0 | 0 | 0 | 4.759 |
| BRACE2007174 | 0 | 0 | 0 | 0 | 0 | 28.862 | 0 |
| BRACE2007518 | 0 | 0 | 0 | 0 | 0 | 0 | 5.658 |
| BRACE2007641 | 0 | 0 | 0 | 0 | 0 | 11.584 | 15.631 |
| BRACE2007671 | 0 | 0 | 0 | 0 | 0 | 2.856 | 0 |
| BRACE2007768 | 0 | 0 | 0 | 0 | 0 | 0 | 71.560 |
| BRACE2007832 | 0 | 0 | 0 | 0 | 0 | 0 | 31.286 |
| BRACE2007882 | 0 | 0 | 0 | 0 | 0 | 24.334 | 16.418 |
| BRACE2007937 | 0 | 0 | 0 | 0 | 0 | 23.461 | 6.331 |
| BRACE2007952 | 0 | 0 | 0 | 0 | 0 | 0 | 12.088 |
| BRACE2008102 | 0 | 0 | 0 | 0 | 0 | 0 | 59.210 |
| BRACE2008358 | 0 | 0 | 0 | 0 | 0 | 4.186 | 11.298 |
| BRACE2008481 | 0 | 0 | 0 | 0 | 0 | 5.157 | 0 |
| BRACE2008553 | 48.556 | 0 | 0 | 0 | 0 | 0 | 0 |
| BRACE2008653 | 0 | 0 | 0 | 0 | 14.452 | 0 | 0 |
| BRACE2008797 | 0 | 0 | 0 | 0 | 0 | 0 | 30.941 |
| BRACE2008941 | 0 | 0 | 0 | 0 | 0 | 18.463 | 0 |
| BRACE2009037 | 3.409 | 19.603 | 3.937 | 0 | 0 | 0.539 | 0.728 |
| BRACE2009044 | 0 | 0 | 0 | 0 | 0 | 18.536 | 0 |
| BRACE2009045 | 0 | 0 | 0 | 0 | 0 | 33.371 | 0 |
| BRACE2009255 | 0 | 0 | 0 | 0 | 0 | 0 | 59.210 |
| BRACE2009517 | 0 | 0 | 0 | 0 | 0 | 0 | 6.761 |
| BRACE2009957 | 0 | 0 | 0 | 0 | 0 | 72.245 | 0 |
| BRACE2010669 | 0 | 0 | 0 | 0 | 0 | 11.777 | 0 |
| BRACE2010828 | 0 | 0 | 0 | 0 | 0 | 72.245 | 0 |
| BRACE2011183 | 0 | 0 | 0 | 0 | 0 | 11.876 | 0 |
| BRACE2012222 | 0 | 0 | 0 | 0 | 0 | 39.421 | 0 |
| BRACE2012291 | 0 | 0 | 0 | 0 | 0 | 72.245 | 0 |
| BRACE2012317 | 0 | 0 | 0 | 0 | 0 | 1.268 | 0 |
| BRACE2012528 | 0 | 0 | 0 | 0 | 0 | 0.876 | 5.911 |
| BRACE2012814 | 0 | 0 | 0 | 0 | 0 | 0 | 7.645 |
| BRACE2013369 | 0 | 0 | 0 | 0 | 0 | 0 | 77.838 |
| BRACE2014257 | 0 | 0 | 0 | 0 | 0 | 22.910 | 0 |
| BRACE2015058 | 0 | 0 | 0 | 0 | 0 | 24.301 | 0 |
| BRACE2017574 | 0 | 0 | 0 | 0 | 0 | 9.465 | 0 |
| BRACE2019147 | 0 | 0 | 0 | 0 | 0 | 7.913 | 5.339 |
| BRACE2019877 | 0 | 0 | 0 | 0 | 0 | 0 | 15.141 |
| BRACE2021014 | 0 | 0 | 0 | 0 | 0 | 10.945 | 0 |
| BRACE2021344 | 0 | 0 | 0 | 0 | 0 | 0 | 77.838 |
| BRACE2022638 | 0 | 0 | 0 | 0 | 0 | 5.508 | 0 |
| BRACE2023718 | 0 | 0 | 0 | 0 | 0 | 29.649 | 0 |
| BRACE2025492 | 0 | 0 | 0 | 0 | 0 | 48.624 | 0 |
| BRACE2025796 | 0 | 0 | 0 | 0 | 0 | 0 | 77.838 |
| BRACE2026054 | 0 | 0 | 0 | 0 | 0 | 42.233 | 0 |
| BRACE2026141 | 0 | 0 | 0 | 0 | 0 | 72.245 | 0 |
| BRACE2030341 | 0 | 0 | 0 | 0 | 0 | 0 | 17.242 |
| BRACE2037295 | 0 | 0 | 0 | 0 | 0 | 0 | 8.177 |
| BRACE2039624 | 11.295 | 0 | 0 | 0 | 0 | 3.575 | 0 |
| BRACE3001113 | 0 | 0 | 0 | 0 | 0 | 48.624 | 0 |
| BRACE3004150 | 0 | 0 | 0 | 45.779 | 0 | 7.175 | 0 |
| BRACE3005938 | 0 | 0 | 0 | 6.490 | 0 | 2.034 | 0 |
| BRACE3010428 | 0 | 0 | 0 | 13.492 | 0 | 6.344 | 2.854 |
| BRACE3010438 | 0 | 0 | 0 | 0 | 0 | 6.616 | 8.928 |
| BRACE3013780 | 55.996 | 0 | 0 | 0 | 0 | 0 | 11.957 |
| BRALZ2008166 | 0 | 0 | 0 | 0 | 0 | 33.831 | 0 |
| BRALZ2009482 | 0 | 0 | 0 | 2.190 | 0 | 0 | 0 |
| BRALZ2011880 | 35.596 | 0 | 0 | 0 | 0 | 0 | 7.601 |
| BRAMY1000173 | 0 | 0 | 0 | 0 | 0 | 19.537 | 0 |
| BRAMY2000181 | 0 | 0 | 0 | 0 | 0 | 64.708 | 0 |
| BRAMY2000354 | 0 | 0 | 0 | 0 | 0 | 5.077 | 3.426 |
| BRAMY2000562 | 0 | 0 | 25.913 | 0 | 0 | 3.550 | 4.791 |
| BRAMY2000893 | 0 | 0 | 41.040 | 0 | 0 | 0 | 0 |
| BRAMY2003929 | 0 | 0 | 0 | 0 | 0 | 78.573 | 0 |
| BRAMY2004363 | 0 | 0 | 0 | 0 | 0 | 3.777 | 0 |
| BRAMY2004521 | 0 | 0 | 0 | 13.969 | 0 | 0 | 0 |
| BRAMY2007078 | 0 | 0 | 0 | 22.410 | 0 | 0 | 0 |
| BRAMY2007244 | 0 | 0 | 0 | 0 | 0 | 0 | 29.217 |
| BRAMY2007567 | 0 | 0 | 0 | 0 | 0 | 0 | 19.344 |
| BRAMY2007613 | 0 | 0 | 0 | 0 | 0 | 4.950 | 3.339 |
| BRAMY2008977 | 0 | 0 | 68.179 | 0 | 0 | 0 | 0 |
| BRAMY2009612 | 0 | 0 | 0 | 0 | 0 | 25.005 | 33.740 |
| BRAMY2009934 | 0 | 0 | 0 | 0 | 0 | 0 | 14.737 |
| BRAMY2015251 | 0 | 0 | 0 | 0 | 0 | 16.971 | 0 |
| BRAMY2019554 | 0 | 0 | 0 | 0 | 0 | 7.371 | 1.989 |
| BRAMY2019989 | 0 | 0 | 0 | 0 | 0 | 0 | 2.404 |
| BRAMY2020106 | 0 | 0 | 0 | 0 | 0 | 20.653 | 0 |
| BRAMY2020427 | 0 | 0 | 0 | 0 | 16.225 | 0 | 0 |
| BRAMY2022493 | 0 | 0 | 0 | 0 | 0 | 0 | 6.613 |
| BRAMY2022786 | 0 | 0 | 0 | 0 | 0 | 2.305 | 6.221 |
| BRAMY2023939 | 0 | 0 | 0 | 0 | 0 | 11.292 | 15.237 |
| BRAMY2024416 | 0 | 0 | 0 | 0 | 0 | 8.359 | 0 |
| BRAMY2024827 | 0 | 0 | 0 | 0 | 0 | 3.686 | 0 |
| BRAMY2026778 | 0 | 0 | 0 | 0 | 0 | 8.951 | 0 |
| BRAMY2028282 | 0 | 0 | 0 | 0 | 0 | 45.089 | 0 |
| BRAMY2028740 | 0 | 0 | 0 | 5.487 | 0 | 1.720 | 6.963 |
| BRAMY2032087 | 0 | 0 | 0 | 0 | 0 | 4.475 | 0 |
| BRAMY2036459 | 0 | 0 | 0 | 0 | 0 | 3.482 | 0 |
| BRAMY2036567 | 0 | 0 | 0 | 0 | 0 | 0 | 18.633 |
| BRAMY2039872 | 0 | 0 | 0 | 0 | 0 | 0 | 49.422 |
| BRAMY2040095 | 0 | 0 | 0 | 0 | 0 | 0 | 2.556 |
| BRAMY3000224 | 0 | 0 | 0 | 0 | 0 | 0 | 71.215 |
| BRAMY3002991 | 45.776 | 0 | 0 | 0 | 0 | 0 | 0 |
| BRAMY3004377 | 0 | 0 | 0 | 0 | 0 | 1.532 | 2.068 |
| BRAMY3005091 | 0 | 0 | 0 | 0 | 0 | 0 | 47.846 |
| BRAMY3008044 | 0 | 0 | 0 | 0 | 0 | 9.942 | 8.944 |
| BRAMY3008335 | 0 | 0 | 0 | 0 | 0 | 0 | 44.280 |
| BRAMY3010492 | 0 | 0 | 0 | 0 | 0 | 0 | 14.357 |
| BRAWH1000037 | 0 | 0 | 0 | 0 | 0 | 0 | 4.546 |
| BRAWH1000040 | 0 | 0 | 0 | 8.110 | 0 | 3.813 | 10.292 |
| BRAWH1000045 | 0 | 0 | 0 | 0 | 0 | 3.699 | 12.477 |
| BRAWH1000093 | 0 | 0 | 6.219 | 2.718 | 0 | 0 | 1.150 |
| BRAWH1000127 | 0 | 0 | 0 | 0 | 0 | 0 | 7.969 |
| BRAWH1000162 | 0 | 0 | 0 | 0 | 0 | 3.372 | 0 |
| BRAWH2000082 | 0 | 0 | 0 | 0 | 0 | 0.709 | 3.825 |
| BRAWH2000169 | 0 | 0 | 0 | 0 | 0 | 19.474 | 0 |
| BRAWH2000274 | 0 | 0 | 0 | 0 | 0 | 3.647 | 24.605 |
| BRAWH2000460 | 0 | 0 | 0 | 0 | 16.724 | 2.255 | 1.521 |
| BRAWH2000522 | 0 | 0 | 0 | 0 | 0 | 3.349 | 4.518 |
| BRAWH2000588 | 0 | 0 | 0 | 0 | 0 | 0 | 12.510 |
| BRAWH2000636 | 0 | 0 | 0 | 0 | 0 | 4.881 | 1.317 |
| BRAWH2000686 | 0 | 0 | 0 | 0 | 0 | 9.033 | 12.189 |
| BRAWH2000839 | 0 | 0 | 0 | 0 | 0 | 3.035 | 2.048 |
| BRAWH2000924 | 0 | 0 | 0 | 0 | 0 | 0.530 | 1.431 |
| BRAWH2001037 | 0 | 0 | 0 | 0 | 0 | 37.045 | 29.993 |
| BRAWH2001092 | 0 | 0 | 0 | 0 | 0 | 1.299 | 3.067 |
| BRAWH2001141 | 0 | 0 | 0 | 0 | 0 | 9.389 | 12.669 |
| BRAWH2001171 | 0 | 0 | 0 | 3.640 | 0 | 3.423 | 4.619 |
| BRAWH2001321 | 0 | 0 | 0 | 0 | 0 | 1.586 | 10.700 |
| BRAWH2001424 | 0 | 0 | 0 | 0 | 0 | 9.406 | 0 |
| BRAWH2001439 | 0 | 0 | 0 | 0 | 0 | 17.108 | 12.825 |
| BRAWH2001484 | 0 | 0 | 0 | 0 | 0 | 19.629 | 0 |
| BRAWH2001662 | 0 | 0 | 0 | 0 | 0 | 8.494 | 34.385 |
| BRAWH2002560 | 0 | 0 | 0 | 0 | 0 | 11.923 | 24.132 |
| BRAWH2002911 | 0 | 0 | 0 | 0 | 0 | 0 | 59.317 |
| BRAWH2003025 | 0 | 0 | 0 | 0 | 0 | 0 | 7.559 |
| BRAWH2003662 | 0 | 0 | 0 | 0 | 0 | 11.450 | 0 |
| BRAWH2003818 | 0 | 0 | 0 | 0 | 0 | 11.183 | 0 |
| BRAWH2004095 | 0 | 0 | 0 | 0 | 0 | 0 | 8.841 |
| BRAWH2005008 | 0 | 0 | 0 | 0 | 0 | 0 | 41.159 |
| BRAWH2005068 | 4.696 | 27.003 | 0 | 0 | 0 | 0 | 0 |
| BRAWH2005074 | 0 | 0 | 0 | 0 | 0 | 64.881 | 0 |
| BRAWH2005661 | 0 | 0 | 0 | 0 | 0 | 0 | 3.985 |
| BRAWH2006450 | 0 | 0 | 0 | 0 | 0 | 0 | 12.517 |
| BRAWH2006493 | 0 | 0 | 0 | 0 | 0 | 0 | 12.680 |
| BRAWH2006996 | 0 | 0 | 0 | 0 | 0 | 6.284 | 0 |
| BRAWH2007605 | 0 | 0 | 0 | 0 | 0 | 0 | 10.227 |
| BRAWH2007664 | 0 | 0 | 0 | 0 | 0 | 18.903 | 0 |
| BRAWH2007800 | 0 | 0 | 0 | 0 | 0 | 4.340 | 0 |
| BRAWH2008255 | 0 | 0 | 0 | 0 | 0 | 7.805 | 0 |
| BRAWH2008706 | 0 | 0 | 0 | 0 | 0 | 0 | 7.168 |
| BRAWH2008903 | 0 | 0 | 0 | 0 | 0 | 7.811 | 10.540 |
| BRAWH2009360 | 0 | 0 | 0 | 50.998 | 0 | 0 | 0 |
| BRAWH2010329 | 0 | 0 | 0 | 0 | 0 | 13.868 | 0 |
| BRAWH2010980 | 0 | 0 | 0 | 0 | 0 | 0 | 17.615 |
| BRAWH2011343 | 0 | 0 | 0 | 0 | 0 | 0 | 17.488 |
| BRAWH2011812 | 0 | 0 | 0 | 0 | 0 | 25.021 | 0 |
| BRAWH2012161 | 0 | 0 | 0 | 0 | 0 | 14.227 | 0 |
| BRAWH2012164 | 0 | 0 | 0 | 0 | 0 | 38.493 | 0 |
| BRAWH2012277 | 0 | 0 | 0 | 0 | 0 | 0 | 71.370 |
| BRAWH2012827 | 0 | 0 | 0 | 0 | 0 | 16.614 | 0 |
| BRAWH2012866 | 0 | 0 | 0 | 0 | 0 | 15.708 | 0 |
| BRAWH2014188 | 0 | 0 | 0 | 0 | 0 | 2.645 | 0 |
| BRAWH2014511 | 0 | 0 | 0 | 0 | 0 | 7.938 | 0 |
| BRAWH2014645 | 0 | 0 | 0 | 0 | 0 | 0 | 2.635 |
| BRAWH2015247 | 0 | 0 | 0 | 0 | 0 | 0 | 26.025 |
| BRAWH2016724 | 0 | 0 | 0 | 0 | 0 | 64.881 | 0 |
| BRAWH2017002 | 0 | 0 | 0 | 0 | 0 | 22.987 | 6.203 |
| BRAWH2017542 | 0 | 0 | 0 | 0 | 0 | 64.881 | 0 |
| BRAWH2017685 | 0 | 0 | 0 | 0 | 0 | 1.875 | 2.530 |
| BRAWH2018526 | 0 | 0 | 0 | 0 | 0 | 0 | 0.767 |
| BRAWH3000088 | 0 | 0 | 0 | 0 | 0 | 19.234 | 0 |
| BRAWH3002853 | 0 | 0 | 0 | 0 | 0 | 1.367 | 1.844 |
| BRAWH3005146 | 0 | 0 | 0 | 0 | 0 | 9.354 | 12.622 |
| BRAWH3010461 | 0 | 0 | 0 | 0 | 0 | 0 | 5.121 |
| BRAWH3016271 | 0 | 0 | 0 | 0 | 0 | 19.929 | 0 |
| BRAWH3017259 | 0 | 0 | 0 | 0 | 0 | 12.928 | 0 |
| BRAWH3018063 | 30.813 | 0 | 0 | 0 | 0 | 0 | 0 |
| BRAWH3021574 | 0 | 0 | 0 | 0 | 0 | 8.683 | 0 |
| BRCAN1000168 | 0 | 0 | 0 | 0 | 0 | 0 | 3.517 |
| BRCAN2000563 | 0 | 0 | 0 | 0 | 0 | 0 | 32.782 |
| BRCAN2003944 | 0 | 0 | 0 | 1.660 | 0 | 0 | 1.404 |
| BRCAN2009432 | 0 | 0 | 0 | 7.760 | 36.081 | 0 | 0 |
| BRCAN2010665 | 0 | 0 | 0 | 0 | 0 | 0 | 8.147 |
| BRCAN2024572 | 0 | 0 | 0 | 0 | 0 | 10.091 | 20.425 |
| BRCAN2026197 | 0 | 0 | 0 | 0 | 0 | 2.884 | 0 |
| BRCAN2028036 | 0 | 0 | 0 | 0 | 0 | 4.460 | 0 |
| BRCOC2000004 | 0 | 0 | 0 | 0 | 0 | 0 | 4.487 |
| BRCOC2010123 | 0 | 0 | 14.772 | 6.456 | 0 | 0 | 0 |
| BRCOC2019910 | 0 | 0 | 0 | 0 | 0 | 2.309 | 3.116 |
| BRHIP1000104 | 0 | 0 | 0 | 0 | 0 | 1.208 | 1.629 |
| BRHIP1000108 | 0 | 0 | 0 | 0 | 0 | 7.962 | 10.743 |
| BRHIP1000174 | 0 | 0 | 0 | 0 | 0 | 9.366 | 25.278 |
| BRHIP1000258 | 0 | 0 | 0 | 0 | 0 | 47.766 | 0 |
| BRHIP2000312 | 0 | 0 | 0 | 0 | 0 | 0 | 16.166 |
| BRHIP2000534 | 0 | 0 | 4.615 | 14.119 | 0 | 0 | 0.853 |
| BRHIP2000553 | 0 | 0 | 0 | 0 | 0 | 4.747 | 0 |
| BRHIP2001099 | 0 | 0 | 0 | 0 | 0 | 12.807 | 0 |
| BRHIP2001805 | 0 | 0 | 0 | 85.243 | 0 | 0 | 0 |
| BRHIP2002339 | 0 | 0 | 0 | 0 | 0 | 28.997 | 0 |
| BRHIP2007305 | 0 | 0 | 0 | 6.671 | 0 | 2.091 | 0 |
| BRHIP2007307 | 0 | 0 | 0 | 0 | 0 | 0 | 70.959 |
| BRHIP2007741 | 0 | 0 | 0 | 0 | 0 | 3.207 | 4.327 |
| BRHIP2010217 | 0 | 0 | 0 | 0 | 0 | 0 | 13.922 |
| BRHIP2011080 | 0 | 0 | 0 | 0 | 0 | 64.423 | 0 |
| BRHIP2012054 | 0 | 0 | 0 | 0 | 0 | 0 | 70.959 |
| BRHIP2013699 | 0 | 0 | 0 | 0 | 0 | 39.421 | 0 |
| BRHIP2015224 | 0 | 0 | 0 | 0 | 0 | 0 | 22.681 |
| BRHIP2015245 | 0 | 0 | 0 | 0 | 0 | 0 | 5.918 |
| BRHIP2015679 | 29.287 | 0 | 0 | 0 | 0 | 0 | 0 |
| BRHIP2017642 | 0 | 0 | 0 | 0 | 0 | 47.719 | 0 |
| BRHIP2018712 | 0 | 0 | 0 | 0 | 0 | 11.224 | 0 |
| BRHIP2019186 | 0 | 0 | 0 | 0 | 0 | 7.292 | 9.840 |
| BRHIP2020573 | 0 | 0 | 0 | 0 | 0 | 33.438 | 0 |
| BRHIP2021495 | 0 | 0 | 0 | 0 | 0 | 11.849 | 15.989 |
| BRHIP2021762 | 0 | 0 | 0 | 0 | 0 | 5.147 | 0 |
| BRHIP2021858 | 0 | 0 | 0 | 0 | 0 | 6.182 | 0 |
| BRHIP2022986 | 0 | 0 | 0 | 0 | 0 | 15.770 | 0 |
| BRHIP2023292 | 0 | 0 | 0 | 0 | 0 | 64.423 | 0 |
| BRHIP2023773 | 0 | 0 | 0 | 0 | 0 | 0 | 70.959 |
| BRHIP2023888 | 0 | 0 | 0 | 4.337 | 0 | 0.680 | 0 |
| BRHIP2024742 | 0 | 0 | 0 | 0 | 0 | 2.304 | 0 |
| BRHIP2026214 | 0 | 0 | 0 | 0 | 0 | 64.423 | 0 |
| BRHIP2027077 | 0 | 0 | 0 | 11.904 | 0 | 3.732 | 0 |
| BRHIP3000017 | 0 | 0 | 0 | 0 | 0 | 8.146 | 0 |
| BRHIP3000111 | 0 | 0 | 0 | 0 | 28.053 | 0 | 2.552 |
| BRHIP3002141 | 0 | 0 | 0 | 0 | 0 | 0 | 39.113 |
| BRHIP3002691 | 0 | 0 | 0 | 0 | 0 | 15.322 | 0 |
| BRHIP3006717 | 0 | 0 | 0 | 0 | 0 | 3.144 | 21.215 |
| BRHIP3007586 | 0 | 0 | 0 | 0 | 0 | 6.375 | 0 |
| BRHIP3009099 | 0 | 0 | 0 | 0 | 0 | 64.423 | 0 |
| BRHIP3012997 | 0 | 0 | 0 | 0 | 0 | 1.712 | 2.310 |
| BRHIP3017855 | 0 | 0 | 0 | 0 | 0 | 0 | 9.545 |
| BRHIP3020046 | 0 | 68.001 | 0 | 0 | 0 | 0 | 0 |
| BRHIP3021019 | 0 | 0 | 0 | 0 | 0 | 14.308 | 19.306 |
| BRSSN1000056 | 70.732 | 0 | 0 | 0 | 0 | 0 | 0 |
| BRSSN2000312 | 0 | 0 | 0 | 0 | 0 | 19.877 | 0 |
| BRSSN2000498 | 0 | 0 | 0 | 0 | 0 | 2.022 | 2.728 |
| BRSSN2000561 | 0 | 0 | 0 | 0 | 0 | 11.584 | 15.631 |
| BRSSN2000566 | 0 | 0 | 0 | 0 | 0 | 0 | 24.251 |
| BRSSN2007338 | 0 | 0 | 0 | 0 | 0 | 0 | 18.425 |
| BRSSN2007464 | 0 | 0 | 55.181 | 0 | 0 | 0 | 0 |
| BRSSN2011653 | 0 | 0 | 0 | 0 | 0 | 18.308 | 0 |
| BRSSN2011843 | 0 | 0 | 0 | 0 | 0 | 22.111 | 0 |
| BRSSN2013696 | 41.698 | 0 | 0 | 0 | 0 | 0 | 0 |
| BRSSN2015238 | 0 | 0 | 0 | 0 | 0 | 0 | 8.534 |
| BRSTN1000083 | 0 | 0 | 0 | 0 | 0 | 4.718 | 1.592 |
| BRSTN2000220 | 0 | 0 | 0 | 0 | 0 | 0 | 8.553 |
| BRSTN2010089 | 0 | 0 | 0 | 0 | 0 | 0 | 16.447 |
| BRSTN2010750 | 0 | 0 | 0 | 0 | 0 | 0 | 25.357 |
| BRSTN2011961 | 0 | 0 | 0 | 0 | 0 | 0 | 0.722 |
| BRSTN2012069 | 0.442 | 0 | 0.128 | 0.223 | 0 | 0.157 | 0.401 |
| BRSTN2012089 | 0 | 0 | 0 | 0 | 0 | 0 | 14.460 |
| BRSTN2012174 | 0 | 0 | 0 | 0 | 0 | 0.374 | 0 |
| BRSTN2013502 | 0 | 0 | 0 | 0 | 0 | 0 | 6.059 |
| BRSTN2016470 | 2.555 | 0 | 0 | 0 | 0 | 0 | 0.546 |
| BRSTN2016892 | 0 | 0 | 0 | 0 | 0 | 0 | 7.439 |
| BRSTN2017104 | 0 | 0 | 0 | 0 | 53.428 | 0 | 0 |
| BRTHA2005864 | 0 | 0 | 0 | 0 | 0 | 5.995 | 0 |
| BRTHA2008502 | 0 | 0 | 0 | 0 | 0 | 0 | 53.934 |
| BRTHA2012714 | 0 | 0 | 0 | 7.387 | 0 | 0 | 0 |
| BRTHA2016577 | 0 | 0 | 0 | 0 | 0 | 0 | 5.882 |
| BRTHA2027229 | 0 | 61.446 | 0 | 0 | 0 | 1.691 | 0 |
| BRTHA2033155 | 0 | 0 | 0 | 0 | 0 | 0 | 10.602 |
| BRTHA3009090 | 0 | 0 | 0 | 0 | 0 | 0 | 29.341 |
| BRTHA3009858 | 0 | 0 | 0 | 0 | 0 | 0 | 57.818 |
| BRTHA3010530 | 0 | 0 | 0 | 0 | 0 | 25.829 | 0 |
| BRTHA3011194 | 0 | 0 | 0 | 0 | 0 | 6.530 | 8.811 |
| BRTHA3022641 | 0 | 0 | 0 | 0 | 0 | 32.682 | 0 |
| CD34C3000252 | 0 | 0 | 0 | 0 | 0 | 0 | 4.092 |
| CTONG1000052 | 0 | 0 | 0 | 0 | 0 | 4.465 | 3.012 |
| CTONG1000087 | 0 | 0 | 0 | 6.163 | 0 | 1.932 | 2.607 |
| CTONG1000094 | 0 | 0 | 0 | 0 | 0 | 10.792 | 16.505 |
| CTONG1000218 | 0 | 0 | 0 | 2.838 | 0 | 0.890 | 4.801 |
| CTONG1000241 | 0 | 0 | 0 | 0 | 0 | 3.919 | 5.288 |
| CTONG2000452 | 0 | 0 | 0 | 0 | 0 | 5.381 | 0 |
| CTONG2000508 | 0 | 0 | 0 | 0 | 0 | 0 | 1.969 |
| CTONG2001139 | 0 | 0 | 0 | 0 | 0 | 49.796 | 0 |
| CTONG2001320 | 0 | 0 | 0 | 31.070 | 0 | 9.740 | 0 |
| CTONG2001748 | 0 | 0 | 0 | 0 | 0 | 11.585 | 5.211 |
| CTONG2001911 | 0 | 0 | 0 | 0 | 0 | 0 | 37.668 |
| CTONG2002073 | 0 | 0 | 0 | 0 | 0 | 0 | 7.303 |
| CTONG2002270 | 0 | 0 | 0 | 3.126 | 0 | 0 | 1.322 |
| CTONG2002453 | 0 | 0 | 0 | 0 | 0 | 14.342 | 0 |
| CTONG2002744 | 0 | 0 | 7.941 | 3.471 | 0 | 2.176 | 1.468 |
| CTONG2002803 | 0 | 0 | 0 | 0 | 0 | 2.720 | 0 |
| CTONG2002820 | 0 | 0 | 0 | 0 | 0 | 20.779 | 0 |
| CTONG2002903 | 0 | 0 | 0 | 18.153 | 0 | 0 | 0 |
| CTONG2003094 | 0 | 0 | 0 | 0 | 0 | 3.385 | 0 |
| CTONG2003293 | 0 | 0 | 0 | 0 | 0 | 0 | 23.043 |
| CTONG2003298 | 0 | 0 | 9.785 | 8.553 | 0 | 4.022 | 0 |
| CTONG2003524 | 0 | 0 | 0 | 0 | 0 | 5.954 | 0 |
| CTONG2005290 | 0 | 0 | 0 | 0 | 0 | 0 | 57.236 |
| CTONG2005553 | 0 | 0 | 0 | 0 | 0 | 2.646 | 0 |
| CTONG2006235 | 0 | 0 | 0 | 0 | 0 | 3.026 | 4.084 |
| CTONG2006412 | 0 | 0 | 0 | 0 | 0 | 11.468 | 0 |
| CTONG2006562 | 0 | 0 | 0 | 0 | 0 | 2.587 | 0 |
| CTONG2006709 | 0 | 0 | 0 | 0 | 0 | 4.778 | 0 |
| CTONG2006798 | 0 | 0 | 0 | 56.866 | 0 | 0 | 0 |
| CTONG2006932 | 0 | 0 | 0 | 6.485 | 0 | 0 | 0 |
| CTONG2007500 | 0 | 0 | 0 | 0 | 0 | 1.389 | 0 |
| CTONG2007586 | 0 | 0 | 0 | 0 | 0 | 11.104 | 0 |
| CTONG2007776 | 0 | 0 | 0 | 0 | 0 | 0 | 10.603 |
| CTONG2008398 | 0 | 0 | 0 | 4.506 | 0 | 0 | 0 |
| CTONG2008506 | 0 | 0 | 0 | 0 | 0 | 1.738 | 9.383 |
| CTONG2008518 | 0 | 0 | 0 | 22.697 | 0 | 7.115 | 0 |
| CTONG2008521 | 0 | 0 | 0 | 0 | 0 | 56.173 | 0 |
| CTONG2008792 | 0 | 0 | 0 | 0 | 0 | 0 | 36.634 |
| CTONG2010566 | 0 | 0 | 0 | 0 | 0 | 50.102 | 0 |
| CTONG2010649 | 0 | 0 | 0 | 0 | 0 | 8.667 | 7.796 |
| CTONG2010821 | 0 | 47.973 | 0 | 0 | 0 | 0 | 3.563 |
| CTONG2011801 | 0 | 0 | 0 | 0 | 0 | 16.411 | 7.381 |
| CTONG2012001 | 0 | 0 | 0 | 0 | 0 | 0 | 35.651 |
| CTONG2012050 | 0 | 0 | 0 | 41.864 | 0 | 0 | 0 |
| CTONG2012401 | 0 | 0 | 0 | 0 | 0 | 15.543 | 0 |
| CTONG2012452 | 0 | 0 | 0 | 0 | 0 | 0.658 | 0 |
| CTONG2012996 | 0 | 0 | 0 | 0 | 0 | 5.394 | 0 |
| CTONG2013348 | 0 | 0 | 0 | 0 | 0 | 10.163 | 0 |
| CTONG2013381 | 0 | 0 | 0 | 0 | 0 | 5.796 | 0 |
| CTONG2013907 | 0 | 0 | 0 | 0 | 0 | 7.966 | 0 |
| CTONG2013985 | 0 | 0 | 0 | 0 | 0 | 10.819 | 0 |
| CTONG2014165 | 0 | 0 | 64.925 | 0 | 0 | 0 | 0 |
| CTONG2014389 | 0 | 0 | 0 | 15.567 | 0 | 4.880 | 0 |
| CTONG2014898 | 0 | 0 | 0 | 0 | 0 | 10.656 | 0 |
| CTONG2015091 | 0 | 0 | 0 | 4.786 | 0 | 3.001 | 2.024 |
| CTONG2015204 | 0 | 0 | 0 | 0 | 0 | 28.022 | 0 |
| CTONG2016056 | 0 | 0 | 0 | 0 | 0 | 3.674 | 0 |
| CTONG2016408 | 0 | 0 | 49.924 | 0 | 0 | 0 | 0 |
| CTONG2016575 | 0 | 0 | 6.704 | 2.930 | 0 | 7.348 | 0 |
| CTONG2016824 | 0 | 0 | 0 | 0 | 0 | 15.727 | 0 |
| CTONG2018343 | 0 | 0 | 0 | 0 | 0 | 56.281 | 0 |
| CTONG2018637 | 0 | 0 | 0 | 0 | 0 | 12.120 | 0 |
| CTONG2018739 | 0 | 0 | 0 | 0 | 0 | 14.319 | 0 |
| CTONG2018843 | 0 | 0 | 0 | 0 | 0 | 0 | 27.676 |
| CTONG2018898 | 0 | 0 | 0 | 0 | 0 | 16.905 | 7.203 |
| CTONG2019063 | 0 | 0 | 0 | 0 | 0 | 39.224 | 0 |
| CTONG2019248 | 0 | 0 | 0 | 0 | 0 | 27.950 | 0 |
| CTONG2020411 | 0 | 0 | 0 | 0 | 0 | 12.719 | 0 |
| CTONG2020484 | 0 | 0 | 0 | 0 | 0 | 1.086 | 0 |
| CTONG2020522 | 0 | 0 | 0 | 0 | 0 | 42.337 | 0 |
| CTONG2020560 | 0 | 0 | 20.228 | 0 | 0 | 2.771 | 0 |
| CTONG2020582 | 0 | 0 | 0 | 0 | 0 | 3.545 | 0 |
| CTONG2022835 | 0 | 0 | 0 | 0 | 0 | 3.169 | 0 |
| CTONG2028124 | 0 | 0 | 0 | 0 | 0 | 1.857 | 2.506 |
| CTONG3001123 | 0 | 0 | 0 | 46.246 | 0 | 0 | 0 |
| CTONG3002835 | 12.826 | 0 | 14.814 | 0 | 0 | 0 | 0 |
| CTONG3008894 | 8.487 | 0 | 0 | 0 | 0 | 2.686 | 0 |
| CTONG3009239 | 0 | 0 | 0 | 0 | 0 | 8.002 | 0 |
| D30ST1000066 | 100 | 0 | 0 | 0 | 0 | 0 | 0 |
| D30ST1000109 | 62.515 | 0 | 24.068 | 0 | 0 | 0 | 0 |
| D30ST1000238 | 100 | 0 | 0 | 0 | 0 | 0 | 0 |
| D30ST1000267 | 50.415 | 0 | 0 | 0 | 0 | 7.978 | 10.765 |
| D30ST1000270 | 30.165 | 0 | 0 | 0 | 0 | 0 | 0 |
| D30ST2000184 | 100 | 0 | 0 | 0 | 0 | 0 | 0 |
| D30ST2000618 | 100 | 0 | 0 | 0 | 0 | 0 | 0 |
| D30ST2000654 | 100 | 0 | 0 | 0 | 0 | 0 | 0 |
| D30ST2000734 | 93.334 | 0 | 0 | 0 | 0 | 0 | 0 |
| D30ST2001328 | 100 | 0 | 0 | 0 | 0 | 0 | 0 |
| D30ST2001669 | 100 | 0 | 0 | 0 | 0 | 0 | 0 |
| D30ST2001670 | 43.561 | 0 | 0 | 0 | 0 | 0 | 0 |
| D30ST2001788 | 34.047 | 0 | 0 | 0 | 0 | 0 | 0 |
| D30ST2002068 | 35.114 | 28.845 | 17.381 | 0 | 0 | 0 | 0 |
| D30ST2002182 | 86.773 | 0 | 0 | 0 | 0 | 0 | 0 |
| D30ST2002223 | 100 | 0 | 0 | 0 | 0 | 0 | 0 |
| D30ST2002262 | 100 | 0 | 0 | 0 | 0 | 0 | 0 |
| D30ST2002272 | 100 | 0 | 0 | 0 | 0 | 0 | 0 |
| D30ST2002417 | 100 | 0 | 0 | 0 | 0 | 0 | 0 |
| D30ST2002436 | 100 | 0 | 0 | 0 | 0 | 0 | 0 |
| D30ST2002648 | 17.515 | 0 | 0 | 0 | 0 | 0 | 0 |
| D30ST2003024 | 94.698 | 0 | 0 | 0 | 0 | 0 | 0 |
| D30ST2003331 | 100 | 0 | 0 | 0 | 0 | 0 | 0 |
| D30ST2003607 | 46.404 | 0 | 53.596 | 0 | 0 | 0 | 0 |
| D30ST2003797 | 100 | 0 | 0 | 0 | 0 | 0 | 0 |
| D30ST2004637 | 100 | 0 | 0 | 0 | 0 | 0 | 0 |
| D30ST3000169 | 28.566 | 0 | 19.796 | 0 | 0 | 0 | 0 |
| D30ST3000195 | 100 | 0 | 0 | 0 | 0 | 0 | 0 |
| D30ST3000258 | 72.574 | 0 | 0 | 0 | 0 | 0 | 0 |
| D30ST3000291 | 93.334 | 0 | 0 | 0 | 0 | 0 | 0 |
| D30ST3000388 | 100 | 0 | 0 | 0 | 0 | 0 | 0 |
| D30ST3000493 | 100 | 0 | 0 | 0 | 0 | 0 | 0 |
| D60ST2000127 | 0 | 100 | 0 | 0 | 0 | 0 | 0 |
| D60ST2000169 | 0 | 100 | 0 | 0 | 0 | 0 | 0 |
| D60ST2000245 | 0 | 100 | 0 | 0 | 0 | 0 | 0 |
| D60ST2000358 | 0 | 95.400 | 0 | 0 | 0 | 0 | 0 |
| D60ST2000464 | 0 | 73.552 | 0 | 0 | 0 | 0 | 0 |
| D60ST2000507 | 0 | 100 | 0 | 0 | 0 | 0 | 0 |
| D90ST2000031 | 0 | 0 | 63.705 | 0 | 0 | 0 | 0 |
| D90ST2000278 | 0 | 0 | 100 | 0 | 0 | 0 | 0 |
| D90ST2000440 | 0 | 0 | 100 | 0 | 0 | 0 | 0 |
| D90ST2000869 | 0 | 0 | 100 | 0 | 0 | 0 | 0 |
| D90ST2001319 | 0 | 0 | 100 | 0 | 0 | 0 | 0 |
| D90ST2001433 | 0 | 0 | 100 | 0 | 0 | 0 | 0 |
| D90ST2002397 | 0 | 0 | 37.837 | 0 | 0 | 0 | 0 |
| D90ST2002608 | 0 | 0 | 30.042 | 0 | 0 | 0 | 0 |
| D90ST2002673 | 0 | 0 | 19.695 | 0 | 0 | 0 | 0 |
| D90ST2003106 | 0 | 0 | 93.096 | 0 | 0 | 0 | 0 |
| D90ST2003108 | 0 | 0 | 65.767 | 0 | 0 | 0 | 0 |
| D90ST2003137 | 0 | 0 | 100 | 0 | 0 | 0 | 0 |
| D90ST2003397 | 0 | 0 | 38.536 | 0 | 0 | 0 | 0 |
| D90ST2003580 | 0 | 0 | 93.047 | 0 | 0 | 0 | 0 |
| D90ST2003594 | 0 | 0 | 100 | 0 | 0 | 0 | 0 |
| D90ST2003762 | 0 | 0 | 29.391 | 0 | 0 | 0 | 0 |
| D90ST2003791 | 0 | 0 | 82.500 | 0 | 0 | 0 | 0 |
| D90ST2003989 | 0 | 0 | 100 | 0 | 0 | 0 | 0 |
| D90ST2004018 | 0 | 0 | 100 | 0 | 0 | 0 | 0 |
| D90ST2004073 | 0 | 0 | 100 | 0 | 0 | 0 | 0 |
| D90ST2004417 | 0 | 0 | 100 | 0 | 0 | 0 | 0 |
| DFNES1000003 | 0 | 0 | 0 | 100 | 0 | 0 | 0 |
| DFNES1000083 | 0 | 0 | 0 | 13.257 | 0 | 0 | 0 |
| DFNES1000107 | 0 | 0 | 0 | 100 | 0 | 0 | 0 |
| DFNES1000150 | 0 | 0 | 0 | 6.581 | 0 | 0 | 0 |
| DFNES1000185 | 0 | 0 | 0 | 7.773 | 0 | 0 | 0 |
| DFNES2000011 | 0 | 0 | 0 | 3.223 | 0 | 0 | 0 |
| DFNES2000143 | 0 | 0 | 0 | 100 | 0 | 0 | 0 |
| DFNES2000146 | 0 | 0 | 0 | 65.605 | 0 | 20.566 | 0 |
| DFNES2000153 | 0 | 0 | 0 | 18.869 | 0 | 0 | 0 |
| DFNES2000212 | 0 | 0 | 0 | 85.399 | 0 | 0 | 0 |
| DFNES2000268 | 0 | 0 | 0 | 6.508 | 0 | 0 | 0 |
| DFNES2000292 | 0 | 0 | 0 | 22.118 | 0 | 0 | 0 |
| DFNES2000335 | 0 | 0 | 0 | 27.920 | 0 | 0 | 0 |
| DFNES2000426 | 0 | 0 | 0 | 74.412 | 0 | 0 | 0 |
| DFNES2000432 | 0 | 0 | 0 | 43.948 | 0 | 0 | 0 |
| DFNES2000443 | 0 | 0 | 0 | 4.000 | 0 | 1.254 | 0 |
| DFNES2000457 | 0 | 0 | 0 | 52.346 | 0 | 0 | 0 |
| DFNES2000471 | 0 | 0 | 0 | 100 | 0 | 0 | 0 |
| DFNES2000526 | 0 | 0 | 0 | 16.286 | 0 | 5.105 | 13.778 |
| DFNES2000913 | 0 | 0 | 0 | 100 | 0 | 0 | 0 |
| DFNES2001091 | 0 | 0 | 0 | 50.577 | 0 | 0 | 0 |
| DFNES2001096 | 0 | 0 | 0 | 45.162 | 0 | 0 | 0 |
| DFNES2001108 | 0 | 0 | 0 | 100 | 0 | 0 | 0 |
| DFNES2001177 | 0 | 0 | 0 | 100 | 0 | 0 | 0 |
| DFNES2001404 | 0 | 0 | 0 | 61.421 | 0 | 0 | 0 |
| DFNES2001564 | 0 | 0 | 0 | 100 | 0 | 0 | 0 |
| DFNES2001565 | 0 | 0 | 0 | 100 | 0 | 0 | 0 |
| DFNES2001647 | 0 | 0 | 0 | 100 | 0 | 0 | 0 |
| DFNES2001800 | 0 | 0 | 0 | 100 | 0 | 0 | 0 |
| DFNES2001829 | 0 | 0 | 0 | 54.385 | 0 | 0 | 0 |
| DFNES2002220 | 0 | 0 | 0 | 100 | 0 | 0 | 0 |
| DFNES2002509 | 0 | 0 | 0 | 29.923 | 0 | 0 | 0 |
| DFNES2002550 | 0 | 0 | 0 | 100 | 0 | 0 | 0 |
| DFNES2002588 | 0 | 0 | 0 | 100 | 0 | 0 | 0 |
| DFNES2002817 | 0 | 0 | 0 | 100 | 0 | 0 | 0 |
| DFNES2002966 | 0 | 0 | 0 | 90.016 | 0 | 0 | 0 |
| DFNES2003081 | 0 | 0 | 0 | 46.409 | 0 | 0 | 0 |
| DFNES2003192 | 0 | 0 | 0 | 17.653 | 0 | 0 | 7.467 |
| DFNES2003255 | 0 | 0 | 0 | 100 | 0 | 0 | 0 |
| DFNES2003742 | 0 | 0 | 0 | 61.421 | 0 | 0 | 0 |
| DFNES2004346 | 0 | 0 | 0 | 18.444 | 0 | 0 | 0 |
| DFNES2004354 | 0 | 0 | 0 | 100 | 0 | 0 | 0 |
| DFNES2004371 | 0 | 0 | 0 | 9.298 | 0 | 0 | 0 |
| DFNES2004383 | 0 | 0 | 0 | 100 | 0 | 0 | 0 |
| DFNES2004608 | 0 | 0 | 0 | 44.433 | 0 | 0 | 0 |
| DFNES2004684 | 0 | 0 | 0 | 100 | 0 | 0 | 0 |
| DFNES2005266 | 0 | 0 | 0 | 100 | 0 | 0 | 0 |
| DFNES2005527 | 0 | 0 | 0 | 100 | 0 | 0 | 0 |
| DFNES2005540 | 0 | 0 | 0 | 17.815 | 0 | 0 | 0 |
| DFNES2005690 | 0 | 0 | 0 | 29.920 | 0 | 0 | 0 |
| DFNES2005766 | 0 | 0 | 0 | 10.804 | 0 | 0 | 0 |
| DFNES2005779 | 0 | 0 | 0 | 100 | 0 | 0 | 0 |
| DFNES2005875 | 0 | 0 | 0 | 100 | 0 | 0 | 0 |
| DFNES2006346 | 0 | 0 | 0 | 41.632 | 0 | 13.051 | 0 |
| DFNES2006944 | 0 | 0 | 0 | 100 | 0 | 0 | 0 |
| DFNES2007113 | 0 | 0 | 0 | 39.383 | 0 | 0 | 0 |
| DFNES2007299 | 0 | 0 | 0 | 19.355 | 0 | 0 | 0 |
| DFNES2007332 | 0 | 0 | 0 | 21.872 | 0 | 0 | 0 |
| DFNES2007634 | 0 | 0 | 0 | 10.476 | 0 | 4.926 | 4.432 |
| DFNES2007641 | 0 | 0 | 0 | 62.977 | 0 | 0 | 0 |
| DFNES2007757 | 0 | 0 | 0 | 22.190 | 0 | 0 | 0 |
| DFNES2008088 | 0 | 0 | 0 | 15.493 | 0 | 0 | 0 |
| DFNES2008160 | 0 | 0 | 0 | 26.402 | 0 | 0 | 0 |
| DFNES2008280 | 0 | 0 | 0 | 100 | 0 | 0 | 0 |
| DFNES2008881 | 0 | 0 | 0 | 100 | 0 | 0 | 0 |
| DFNES2009482 | 0 | 0 | 0 | 8.358 | 38.864 | 0 | 3.536 |
| DFNES2010502 | 0 | 0 | 0 | 100 | 0 | 0 | 0 |
| DFNES2011192 | 0 | 0 | 0 | 100 | 0 | 0 | 0 |
| DFNES2011221 | 0 | 0 | 0 | 84.029 | 0 | 0 | 0 |
| DFNES2011239 | 0 | 0 | 0 | 44.035 | 0 | 13.804 | 0 |
| DFNES2011245 | 0 | 0 | 0 | 85.243 | 0 | 0 | 0 |
| DFNES2011380 | 0 | 0 | 0 | 68.891 | 0 | 0 | 0 |
| DFNES2011499 | 0 | 0 | 0 | 36.562 | 0 | 0 | 0 |
| ERLTF1000021 | 0 | 0 | 0 | 0 | 100 | 0 | 0 |
| ERLTF2000324 | 0 | 0 | 0 | 0 | 100 | 0 | 0 |
| ERLTF2000465 | 0 | 0 | 0 | 0 | 100 | 0 | 0 |
| FCBBF1000023 | 0 | 0 | 0 | 0 | 0 | 12.996 | 0 |
| FCBBF1000024 | 0 | 0 | 0 | 12.939 | 0 | 8.112 | 0 |
| FCBBF1000025 | 0 | 0 | 0 | 0 | 0 | 5.912 | 0 |
| FCBBF1000027 | 0 | 0 | 0 | 6.476 | 10.037 | 4.060 | 0.913 |
| FCBBF1000038 | 0 | 0 | 0 | 0 | 0 | 20.755 | 28.007 |
| FCBBF1000053 | 0 | 0 | 0 | 0 | 0 | 14.999 | 0 |
| FCBBF1000061 | 0 | 0 | 0 | 0 | 0 | 13.035 | 0 |
| FCBBF1000063 | 0 | 0 | 0 | 0 | 0 | 11.049 | 0 |
| FCBBF1000069 | 0 | 0 | 0 | 0 | 0 | 100 | 0 |
| FCBBF1000077 | 0 | 0 | 0 | 0 | 0 | 19.451 | 22.208 |
| FCBBF1000115 | 0 | 0 | 0 | 0 | 0 | 37.553 | 0 |
| FCBBF1000117 | 0 | 0 | 0 | 0 | 0 | 100 | 0 |
| FCBBF1000118 | 0 | 0 | 0 | 0 | 0 | 28.015 | 0 |
| FCBBF1000121 | 0 | 0 | 0 | 0 | 0 | 18.354 | 0 |
| FCBBF1000125 | 0 | 0 | 0 | 0 | 0 | 57.554 | 0 |
| FCBBF1000131 | 0 | 0 | 0 | 0 | 0 | 100 | 0 |
| FCBBF1000139 | 0 | 0 | 0 | 0 | 0 | 16.148 | 0 |
| FCBBF1000155 | 0 | 0 | 0 | 0 | 0 | 100 | 0 |
| FCBBF1000157 | 0 | 0 | 0 | 0 | 0 | 100 | 0 |
| FCBBF1000171 | 0 | 0 | 0 | 0 | 0 | 100 | 0 |
| FCBBF1000174 | 0 | 0 | 0 | 0 | 0 | 100 | 0 |
| FCBBF1000182 | 0 | 0 | 0 | 0 | 0 | 15.666 | 0 |
| FCBBF1000197 | 12.472 | 0 | 0 | 6.296 | 0 | 3.947 | 2.663 |
| FCBBF1000198 | 0 | 0 | 0 | 0 | 0 | 100 | 0 |
| FCBBF1000206 | 0 | 0 | 0 | 0 | 0 | 100 | 0 |
| FCBBF1000220 | 0 | 0 | 0 | 0 | 0 | 100 | 0 |
| FCBBF1000232 | 0 | 0 | 0 | 0 | 0 | 100 | 0 |
| FCBBF1000233 | 0 | 0 | 0 | 0 | 0 | 100 | 0 |
| FCBBF1000243 | 0 | 0 | 0 | 0 | 0 | 100 | 0 |
| FCBBF1000270 | 0 | 0 | 0 | 0 | 0 | 100 | 0 |
| FCBBF1000272 | 0 | 0 | 0 | 0 | 0 | 64.423 | 0 |
| FCBBF1000280 | 0 | 0 | 0 | 0 | 0 | 10.435 | 0 |
| FCBBF1000294 | 0 | 0 | 0 | 0 | 0 | 1.989 | 2.684 |
| FCBBF1000297 | 0 | 0 | 0 | 0 | 0 | 100 | 0 |
| FCBBF1000305 | 0 | 0 | 0 | 0 | 0 | 33.142 | 0 |
| FCBBF1000314 | 0 | 0 | 0 | 0 | 0 | 100 | 0 |
| FCBBF1000322 | 0 | 0 | 0 | 0 | 0 | 100 | 0 |
| FCBBF1000349 | 0 | 0 | 0 | 0 | 0 | 100 | 0 |
| FCBBF1000367 | 0 | 0 | 0 | 0 | 0 | 5.613 | 3.787 |
| FCBBF1000374 | 0 | 0 | 0 | 0 | 0 | 73.865 | 0 |
| FCBBF1000376 | 0 | 0 | 0 | 0 | 0 | 78.700 | 0 |
| FCBBF1000377 | 0 | 0 | 0 | 0 | 0 | 39.957 | 26.958 |
| FCBBF1000395 | 0 | 0 | 0 | 0 | 0 | 100 | 0 |
| FCBBF1000397 | 0 | 0 | 0 | 0 | 0 | 100 | 0 |
| FCBBF1000403 | 0 | 0 | 0 | 0 | 0 | 15.495 | 0 |
| FCBBF1000412 | 22.594 | 0 | 0 | 0 | 0 | 3.575 | 0 |
| FCBBF1000425 | 0 | 0 | 0 | 0 | 0 | 9.645 | 0 |
| FCBBF1000437 | 0 | 0 | 0 | 0 | 0 | 23.626 | 31.880 |
| FCBBF1000449 | 0 | 0 | 0 | 0 | 0 | 12.351 | 0 |
| FCBBF1000466 | 0 | 0 | 0 | 0 | 0 | 8.236 | 0 |
| FCBBF1000476 | 0 | 0 | 0 | 0 | 0 | 48.133 | 0 |
| FCBBF1000506 | 0 | 0 | 0 | 0 | 0 | 34.365 | 0 |
| FCBBF1000509 | 0 | 0 | 0 | 0 | 0 | 6.894 | 0 |
| FCBBF1000546 | 0 | 0 | 0 | 0 | 0 | 100 | 0 |
| FCBBF1000550 | 0 | 0 | 0 | 0 | 0 | 100 | 0 |
| FCBBF1000556 | 0 | 0 | 0 | 0 | 0 | 1.969 | 0 |
| FCBBF1000563 | 0 | 0 | 0 | 0 | 0 | 5.550 | 11.232 |
| FCBBF1000574 | 0 | 0 | 0 | 0 | 0 | 7.670 | 0 |
| FCBBF1000581 | 0 | 0 | 0 | 0 | 0 | 100 | 0 |
| FCBBF1000582 | 0 | 0 | 0 | 0 | 0 | 100 | 0 |
| FCBBF1000598 | 0 | 0 | 0 | 0 | 0 | 100 | 0 |
| FCBBF1000604 | 0 | 0 | 0 | 0 | 0 | 100 | 0 |
| FCBBF1000618 | 0 | 0 | 0 | 0 | 0 | 100 | 0 |
| FCBBF1000627 | 0 | 0 | 0 | 0 | 0 | 100 | 0 |
| FCBBF1000671 | 0 | 0 | 0 | 0 | 0 | 25.293 | 0 |
| FCBBF1000675 | 0 | 0 | 0 | 0 | 0 | 23.460 | 31.656 |
| FCBBF1000684 | 0 | 0 | 0 | 0 | 0 | 33.171 | 0 |
| FCBBF1000686 | 0 | 0 | 0 | 0 | 0 | 42.565 | 57.435 |
| FCBBF1000687 | 0 | 0 | 0 | 0 | 0 | 31.919 | 0 |
| FCBBF1000691 | 0 | 0 | 0 | 0 | 0 | 100 | 0 |
| FCBBF1000732 | 0 | 0 | 0 | 0 | 0 | 10.346 | 0 |
| FCBBF1000748 | 0 | 0 | 0 | 0 | 0 | 62.490 | 0 |
| FCBBF1000760 | 0 | 0 | 0 | 0 | 0 | 1.210 | 0 |
| FCBBF2000038 | 0 | 0 | 0 | 0 | 0 | 67.004 | 6.697 |
| FCBBF2000087 | 0 | 0 | 0 | 0 | 0 | 5.330 | 0 |
| FCBBF2000094 | 0 | 0 | 0 | 0 | 0 | 100 | 0 |
| FCBBF2000105 | 0 | 0 | 0 | 0 | 0 | 100 | 0 |
| FCBBF2000123 | 0 | 0 | 0 | 0 | 0 | 100 | 0 |
| FCBBF2000195 | 0 | 0 | 0 | 0 | 0 | 7.679 | 0 |
| FCBBF2000199 | 0 | 0 | 0 | 0 | 0 | 20.674 | 6.974 |
| FCBBF2000214 | 0 | 0 | 0 | 0 | 0 | 100 | 0 |
| FCBBF2000232 | 0 | 0 | 0 | 0 | 0 | 100 | 0 |
| FCBBF2000259 | 0 | 0 | 0 | 0 | 0 | 44.190 | 29.814 |
| FCBBF2000276 | 0 | 0 | 0 | 0 | 0 | 100 | 0 |
| FCBBF2000291 | 0 | 0 | 0 | 0 | 0 | 100 | 0 |
| FCBBF2000362 | 0 | 0 | 0 | 0 | 0 | 15.464 | 0 |
| FCBBF2000379 | 0 | 0 | 0 | 0 | 0 | 100 | 0 |
| FCBBF2000473 | 0 | 0 | 0 | 0 | 0 | 100 | 0 |
| FCBBF2000502 | 0 | 0 | 0 | 0 | 0 | 12.188 | 0 |
| FCBBF2000566 | 0 | 0 | 0 | 0 | 0 | 27.467 | 0 |
| FCBBF2000576 | 0 | 0 | 0 | 0 | 0 | 20.800 | 0 |
| FCBBF2000591 | 0 | 0 | 0 | 0 | 0 | 1.059 | 0 |
| FCBBF2000677 | 0 | 0 | 0 | 0 | 0 | 100 | 0 |
| FCBBF2000678 | 0 | 0 | 0 | 0 | 0 | 100 | 0 |
| FCBBF2000685 | 0 | 0 | 0 | 0 | 0 | 12.569 | 0 |
| FCBBF2000733 | 0 | 0 | 0 | 0 | 0 | 14.642 | 0 |
| FCBBF2000782 | 0 | 0 | 0 | 0 | 0 | 68.901 | 0 |
| FCBBF2000808 | 0 | 0 | 0 | 0 | 0 | 100 | 0 |
| FCBBF2000815 | 0 | 0 | 0 | 0 | 0 | 100 | 0 |
| FCBBF2000825 | 86.338 | 0 | 0 | 0 | 0 | 13.662 | 0 |
| FCBBF2000885 | 0 | 0 | 0 | 0 | 0 | 2.836 | 0 |
| FCBBF2000940 | 0 | 0 | 0 | 0 | 0 | 62.490 | 0 |
| FCBBF2000951 | 0 | 0 | 0 | 0 | 0 | 100 | 0 |
| FCBBF2001001 | 0 | 0 | 0 | 0 | 0 | 17.657 | 0 |
| FCBBF2001088 | 0 | 0 | 0 | 0 | 0 | 29.954 | 0 |
| FCBBF2001105 | 0 | 0 | 0 | 0 | 0 | 5.608 | 0 |
| FCBBF2001116 | 0 | 0 | 0 | 0 | 0 | 100 | 0 |
| FCBBF2001183 | 0 | 0 | 0 | 0 | 0 | 6.110 | 4.122 |
| FCBBF2001188 | 0 | 0 | 0 | 0 | 0 | 100 | 0 |
| FCBBF2001211 | 0 | 0 | 0 | 0 | 0 | 11.839 | 0 |
| FCBBF2001238 | 0 | 0 | 0 | 0 | 0 | 100 | 0 |
| FCBBF2001243 | 0 | 0 | 0 | 0 | 0 | 100 | 0 |
| FCBBF2001291 | 0 | 0 | 0 | 0 | 0 | 10.544 | 0 |
| FCBBF2001299 | 0 | 0 | 0 | 0 | 0 | 83.886 | 0 |
| FCBBF2001309 | 0 | 0 | 0 | 0 | 0 | 32.152 | 10.846 |
| FCBBF2001400 | 0 | 0 | 0 | 0 | 0 | 37.623 | 0 |
| FCBBF2001427 | 0 | 0 | 0 | 0 | 0 | 100 | 0 |
| FCBBF2001480 | 0 | 0 | 0 | 0 | 0 | 44.606 | 0 |
| FCBBF2001529 | 0 | 0 | 0 | 0 | 0 | 100 | 0 |
| FCBBF2001538 | 0 | 0 | 0 | 4.490 | 0 | 4.223 | 1.899 |
| FCBBF2001594 | 0 | 0 | 0 | 0 | 0 | 14.051 | 0 |
| FCBBF2001672 | 0 | 0 | 0 | 0 | 0 | 100 | 0 |
| FCBBF2001675 | 0 | 0 | 0 | 0 | 0 | 100 | 0 |
| FCBBF2001718 | 0 | 0 | 0 | 0 | 0 | 100 | 0 |
| FCBBF2001720 | 0 | 0 | 0 | 0 | 0 | 100 | 0 |
| FCBBF2001817 | 0 | 0 | 0 | 0 | 0 | 44.606 | 0 |
| FCBBF2001868 | 0 | 0 | 0 | 0 | 0 | 100 | 0 |
| FCBBF2002044 | 0 | 0 | 0 | 0 | 0 | 100 | 0 |
| FCBBF2002111 | 0 | 0 | 0 | 0 | 0 | 21.261 | 0 |
| FCBBF2002281 | 0 | 0 | 0 | 0 | 0 | 59.713 | 40.287 |
| FCBBF2002349 | 0 | 0 | 0 | 0 | 0 | 14.587 | 0 |
| FCBBF2002370 | 0 | 0 | 0 | 0 | 0 | 100 | 0 |
| FCBBF2002541 | 0 | 0 | 0 | 0 | 0 | 100 | 0 |
| FCBBF2002626 | 0 | 0 | 0 | 0 | 0 | 72.245 | 0 |
| FCBBF2002801 | 0 | 0 | 0 | 0 | 0 | 14.385 | 0 |
| FCBBF2002897 | 0 | 0 | 0 | 0 | 0 | 100 | 0 |
| FCBBF2002898 | 0 | 0 | 0 | 0 | 0 | 100 | 0 |
| FCBBF2002904 | 0 | 0 | 0 | 0 | 0 | 100 | 0 |
| FCBBF2002928 | 0 | 0 | 0 | 0 | 0 | 5.748 | 0 |
| FCBBF2002953 | 0 | 0 | 0 | 0 | 0 | 100 | 0 |
| FCBBF2003083 | 0 | 0 | 0 | 0 | 0 | 100 | 0 |
| FCBBF2003102 | 0 | 0 | 0 | 0 | 0 | 36.993 | 49.917 |
| FCBBF2003269 | 0 | 0 | 0 | 0 | 0 | 100 | 0 |
| FCBBF2003293 | 0 | 0 | 0 | 0 | 0 | 33.878 | 0 |
| FCBBF2003297 | 0 | 0 | 0 | 0 | 0 | 100 | 0 |
| FCBBF2003395 | 0 | 0 | 0 | 0 | 0 | 100 | 0 |
| FCBBF2003528 | 0 | 0 | 0 | 0 | 0 | 100 | 0 |
| FCBBF2003543 | 0 | 0 | 0 | 0 | 0 | 64.881 | 0 |
| FCBBF2003549 | 0 | 0 | 0 | 0 | 0 | 21.202 | 0 |
| FCBBF2003567 | 0 | 0 | 0 | 0 | 0 | 36.129 | 0 |
| FCBBF2003636 | 0 | 0 | 0 | 0 | 0 | 100 | 0 |
| FCBBF2003823 | 0 | 0 | 0 | 0 | 0 | 100 | 0 |
| FCBBF2003895 | 0 | 0 | 0 | 0 | 0 | 100 | 0 |
| FCBBF2004066 | 0 | 0 | 0 | 0 | 0 | 3.724 | 0 |
| FCBBF2004090 | 0 | 0 | 0 | 0 | 0 | 100 | 0 |
| FCBBF2004138 | 0 | 0 | 0 | 0 | 0 | 100 | 0 |
| FCBBF2004217 | 0 | 0 | 0 | 0 | 0 | 100 | 0 |
| FCBBF2004256 | 0 | 0 | 0 | 0 | 0 | 100 | 0 |
| FCBBF2004373 | 0 | 0 | 0 | 0 | 0 | 43.392 | 0 |
| FCBBF2004448 | 0 | 0 | 0 | 0 | 0 | 100 | 0 |
| FCBBF2004671 | 0 | 0 | 0 | 0 | 0 | 100 | 0 |
| FCBBF2004707 | 0 | 0 | 0 | 0 | 0 | 100 | 0 |
| FCBBF2004788 | 0 | 0 | 0 | 0 | 0 | 100 | 0 |
| FCBBF2005069 | 0 | 0 | 0 | 0 | 0 | 100 | 0 |
| FCBBF2005122 | 0 | 0 | 0 | 0 | 0 | 100 | 0 |
| FCBBF2005428 | 0 | 0 | 0 | 0 | 0 | 72.245 | 0 |
| FCBBF2005439 | 0 | 0 | 0 | 0 | 0 | 100 | 0 |
| FCBBF2005538 | 0 | 0 | 0 | 0 | 0 | 100 | 0 |
| FCBBF2005637 | 0 | 0 | 0 | 0 | 76.147 | 5.134 | 0 |
| FCBBF2005645 | 0 | 0 | 0 | 0 | 0 | 100 | 0 |
| FCBBF2005658 | 0 | 0 | 0 | 0 | 0 | 14.379 | 0 |
| FCBBF2005733 | 0 | 0 | 0 | 0 | 0 | 59.793 | 0 |
| FCBBF2005909 | 0 | 0 | 0 | 0 | 0 | 11.530 | 15.558 |
| FCBBF2005966 | 0 | 0 | 0 | 0 | 0 | 17.497 | 0 |
| FCBBF2006131 | 0 | 0 | 0 | 0 | 0 | 100 | 0 |
| FCBBF2006380 | 0 | 0 | 0 | 0 | 0 | 60.769 | 0 |
| FCBBF2006418 | 0 | 0 | 0 | 0 | 0 | 100 | 0 |
| FCBBF2006452 | 0 | 0 | 0 | 0 | 0 | 100 | 0 |
| FCBBF2006634 | 0 | 0 | 63.649 | 0 | 0 | 8.720 | 0 |
| FCBBF2006781 | 0 | 0 | 0 | 0 | 0 | 11.690 | 0 |
| FCBBF2007012 | 0 | 0 | 0 | 0 | 0 | 100 | 0 |
| FCBBF2007186 | 0 | 0 | 0 | 0 | 0 | 100 | 0 |
| FCBBF2007265 | 0 | 0 | 0 | 0 | 0 | 100 | 0 |
| FCBBF2007302 | 0 | 0 | 0 | 0 | 0 | 43.392 | 0 |
| FCBBF2007556 | 0 | 0 | 0 | 0 | 0 | 16.883 | 0 |
| FCBBF2007610 | 0 | 0 | 0 | 0 | 0 | 100 | 0 |
| FCBBF2007633 | 0 | 0 | 0 | 0 | 0 | 100 | 0 |
| FCBBF2007840 | 0 | 0 | 0 | 0 | 0 | 46.021 | 0 |
| FCBBF3000102 | 0 | 0 | 0 | 0 | 0 | 22.924 | 0 |
| FCBBF3000110 | 0 | 0 | 0 | 0 | 0 | 23.863 | 0 |
| FCBBF3000115 | 0 | 0 | 0 | 0 | 0 | 7.373 | 0 |
| FCBBF3000120 | 0 | 0 | 0 | 0 | 0 | 100 | 0 |
| FCBBF3000168 | 0 | 0 | 0 | 0 | 0 | 15.041 | 0 |
| FCBBF3000175 | 0 | 0 | 56.620 | 0 | 0 | 15.515 | 0 |
| FCBBF3000227 | 0 | 0 | 0 | 0 | 0 | 20.773 | 0 |
| FCBBF3000228 | 0 | 0 | 0 | 0 | 0 | 16.557 | 0 |
| FCBBF3000229 | 0 | 0 | 0 | 0 | 0 | 17.839 | 24.071 |
| FCBBF3000233 | 0 | 0 | 0 | 0 | 0 | 100 | 0 |
| FCBBF3000269 | 0 | 0 | 0 | 0 | 0 | 56.549 | 0 |
| FCBBF3000361 | 0 | 0 | 0 | 0 | 0 | 100 | 0 |
| FCBBF3000367 | 0 | 0 | 0 | 0 | 0 | 100 | 0 |
| FCBBF3000388 | 0 | 0 | 0 | 0 | 0 | 22.816 | 0 |
| FCBBF3000434 | 0 | 0 | 0 | 0 | 0 | 6.841 | 0 |
| FCBBF3000462 | 0 | 0 | 0 | 0 | 0 | 100 | 0 |
| FCBBF3000473 | 0 | 0 | 0 | 0 | 0 | 100 | 0 |
| FCBBF3000501 | 0 | 0 | 0 | 0 | 0 | 100 | 0 |
| FCBBF3000518 | 0 | 0 | 0 | 0 | 0 | 100 | 0 |
| FCBBF3000536 | 0 | 0 | 0 | 0 | 0 | 4.925 | 0 |
| FCBBF3000550 | 0 | 0 | 0 | 0 | 0 | 100 | 0 |
| FCBBF3000847 | 0 | 0 | 0 | 0 | 0 | 100 | 0 |
| FCBBF3001018 | 0 | 0 | 0 | 0 | 0 | 100 | 0 |
| FCBBF3001020 | 0 | 0 | 0 | 0 | 0 | 100 | 0 |
| FCBBF3001081 | 0 | 0 | 0 | 0 | 0 | 9.702 | 0 |
| FCBBF3001235 | 0 | 0 | 0 | 0 | 0 | 100 | 0 |
| FCBBF3001281 | 0 | 0 | 0 | 0 | 0 | 37.617 | 0 |
| FCBBF3001302 | 0 | 0 | 0 | 0 | 91.087 | 6.141 | 0 |
| FCBBF3001377 | 0 | 0 | 0 | 0 | 0 | 19.023 | 25.669 |
| FCBBF3001470 | 0 | 0 | 0 | 0 | 0 | 2.508 | 0 |
| FCBBF3001594 | 0 | 0 | 0 | 0 | 0 | 100 | 0 |
| FCBBF3001632 | 0 | 0 | 0 | 0 | 0 | 2.852 | 3.848 |
| FCBBF3001657 | 0 | 0 | 0 | 0 | 0 | 8.716 | 11.762 |
| FCBBF3001818 | 0 | 0 | 0 | 0 | 0 | 33.656 | 0 |
| FCBBF3001855 | 0 | 0 | 37.763 | 0 | 0 | 31.042 | 0 |
| FCBBF3001890 | 0 | 0 | 0 | 0 | 0 | 15.245 | 13.714 |
| FCBBF3001912 | 0 | 0 | 0 | 0 | 0 | 100 | 0 |
| FCBBF3001914 | 0 | 0 | 0 | 0 | 0 | 100 | 0 |
| FCBBF3001918 | 0 | 0 | 0 | 0 | 0 | 100 | 0 |
| FCBBF3001924 | 0 | 0 | 0 | 0 | 0 | 100 | 0 |
| FCBBF3001977 | 0 | 0 | 0 | 0 | 0 | 13.761 | 0 |
| FCBBF3002163 | 0 | 0 | 0 | 0 | 0 | 100 | 0 |
| FCBBF3002188 | 0 | 0 | 0 | 0 | 0 | 100 | 0 |
| FCBBF3002190 | 0 | 0 | 0 | 0 | 0 | 100 | 0 |
| FCBBF3002268 | 0 | 0 | 0 | 0 | 0 | 100 | 0 |
| FCBBF3002475 | 0 | 0 | 0 | 0 | 0 | 100 | 0 |
| FCBBF3002556 | 0 | 0 | 0 | 0 | 0 | 59.793 | 0 |
| FCBBF3002592 | 0 | 0 | 0 | 0 | 0 | 100 | 0 |
| FCBBF3002658 | 0 | 0 | 0 | 0 | 0 | 100 | 0 |
| FCBBF3002782 | 0 | 0 | 0 | 0 | 0 | 100 | 0 |
| FCBBF3002818 | 0 | 0 | 0 | 0 | 0 | 64.881 | 0 |
| FCBBF3002925 | 0 | 0 | 0 | 0 | 0 | 100 | 0 |
| FCBBF3003216 | 0 | 0 | 0 | 0 | 0 | 100 | 0 |
| FCBBF3003305 | 0 | 0 | 0 | 0 | 0 | 100 | 0 |
| FCBBF3003373 | 0 | 0 | 0 | 0 | 0 | 100 | 0 |
| FCBBF3003435 | 0 | 0 | 0 | 0 | 0 | 33.081 | 0 |
| FCBBF3003475 | 0 | 0 | 0 | 0 | 0 | 100 | 0 |
| FCBBF3003481 | 0 | 0 | 0 | 0 | 0 | 100 | 0 |
| FCBBF3003557 | 0 | 0 | 0 | 0 | 0 | 64.881 | 0 |
| FCBBF3003787 | 0 | 0 | 0 | 0 | 0 | 18.081 | 0 |
| FCBBF3003800 | 0 | 0 | 0 | 2.321 | 0 | 10.916 | 4.910 |
| FCBBF3003902 | 0 | 0 | 0 | 0 | 0 | 43.392 | 0 |
| FCBBF3004021 | 0 | 0 | 0 | 0 | 0 | 19.729 | 0 |
| FCBBF3004041 | 0 | 0 | 0 | 0 | 0 | 6.894 | 0 |
| FCBBF3004261 | 0 | 0 | 0 | 0 | 0 | 33.424 | 0 |
| FCBBF3004323 | 0 | 0 | 0 | 0 | 0 | 20.357 | 41.204 |
| FCBBF3004390 | 0 | 0 | 0 | 0 | 0 | 100 | 0 |
| FCBBF3004409 | 0 | 0 | 0 | 0 | 0 | 59.793 | 0 |
| FCBBF3004473 | 0 | 0 | 0 | 6.771 | 0 | 2.123 | 2.864 |
| FCBBF3004502 | 0 | 0 | 0 | 0 | 0 | 100 | 0 |
| FCBBF3004715 | 0 | 0 | 0 | 0 | 0 | 6.918 | 0 |
| FCBBF3004842 | 0 | 0 | 0 | 0 | 0 | 100 | 0 |
| FCBBF3004847 | 0 | 0 | 0 | 0 | 0 | 60.769 | 0 |
| FCBBF3004955 | 0 | 0 | 0 | 0 | 0 | 28.476 | 19.212 |
| FCBBF3005160 | 0 | 0 | 0 | 0 | 0 | 100 | 0 |
| FCBBF3005218 | 0 | 0 | 0 | 0 | 0 | 100 | 0 |
| FCBBF3005320 | 0 | 0 | 0 | 0 | 0 | 15.119 | 13.601 |
| FCBBF3005330 | 0 | 0 | 0 | 0 | 0 | 6.399 | 1.727 |
| FCBBF3005444 | 0 | 0 | 0 | 0 | 0 | 100 | 0 |
| FCBBF3005497 | 0 | 0 | 0 | 4.633 | 0 | 5.809 | 0 |
| FCBBF3005698 | 0 | 0 | 0 | 0 | 0 | 100 | 0 |
| FCBBF3005729 | 0 | 0 | 0 | 0 | 0 | 100 | 0 |
| FCBBF3006171 | 0 | 0 | 0 | 0 | 0 | 100 | 0 |
| FCBBF3006249 | 0 | 0 | 0 | 0 | 0 | 100 | 0 |
| FCBBF3006288 | 0 | 0 | 0 | 0 | 0 | 100 | 0 |
| FCBBF3006399 | 0 | 0 | 0 | 0 | 0 | 100 | 0 |
| FCBBF3006628 | 0 | 0 | 0 | 0 | 0 | 100 | 0 |
| FCBBF3006821 | 0 | 0 | 0 | 0 | 0 | 10.561 | 0 |
| FCBBF3007077 | 0 | 0 | 0 | 0 | 0 | 100 | 0 |
| FCBBF3007150 | 0 | 0 | 0 | 0 | 0 | 100 | 0 |
| FCBBF3007152 | 0 | 0 | 0 | 0 | 0 | 100 | 0 |
| FCBBF3007242 | 0 | 0 | 0 | 0 | 0 | 100 | 0 |
| FCBBF3007244 | 0 | 0 | 0 | 0 | 0 | 28.681 | 0 |
| FCBBF3007248 | 0 | 0 | 0 | 0 | 0 | 100 | 0 |
| FCBBF3007453 | 0 | 0 | 0 | 0 | 0 | 100 | 0 |
| FCBBF3007462 | 0 | 0 | 0 | 0 | 0 | 64.423 | 0 |
| FCBBF3007540 | 0 | 0 | 0 | 0 | 0 | 100 | 0 |
| FCBBF3007597 | 0 | 0 | 0 | 0 | 0 | 46.137 | 0 |
| FCBBF3007604 | 0 | 0 | 0 | 0 | 0 | 100 | 0 |
| FCBBF3007631 | 0 | 0 | 0 | 0 | 0 | 40.360 | 0 |
| FCBBF3007829 | 0 | 0 | 0 | 0 | 0 | 20.784 | 0 |
| FCBBF3007855 | 0 | 0 | 0 | 0 | 0 | 100 | 0 |
| FCBBF3007859 | 0 | 0 | 0 | 0 | 0 | 12.001 | 0 |
| FCBBF3007860 | 0 | 0 | 0 | 0 | 0 | 18.693 | 0 |
| FCBBF3008073 | 0 | 0 | 0 | 0 | 0 | 100 | 0 |
| FCBBF3008100 | 0 | 0 | 0 | 0 | 0 | 27.561 | 0 |
| FCBBF3008153 | 0 | 0 | 0 | 0 | 0 | 20.215 | 13.639 |
| FCBBF3008159 | 0 | 0 | 0 | 0 | 0 | 38.259 | 0 |
| FCBBF3008251 | 0 | 0 | 0 | 0 | 0 | 100 | 0 |
| FCBBF3008311 | 0 | 0 | 0 | 0 | 0 | 100 | 0 |
| FCBBF3008362 | 0 | 0 | 0 | 0 | 0 | 100 | 0 |
| FCBBF3008382 | 0 | 0 | 0 | 0 | 0 | 19.899 | 26.851 |
| FCBBF3008475 | 0 | 0 | 0 | 0 | 0 | 100 | 0 |
| FCBBF3008556 | 0 | 0 | 0 | 0 | 0 | 73.865 | 0 |
| FCBBF3008644 | 0 | 0 | 0 | 0 | 0 | 15.918 | 0 |
| FCBBF3008689 | 0 | 0 | 0 | 0 | 0 | 34.487 | 0 |
| FCBBF3008748 | 0 | 0 | 0 | 0 | 0 | 100 | 0 |
| FCBBF3008870 | 0 | 0 | 0 | 0 | 0 | 100 | 0 |
| FCBBF3008938 | 0 | 0 | 0 | 0 | 0 | 51.503 | 0 |
| FCBBF3008944 | 0 | 0 | 0 | 0 | 0 | 100 | 0 |
| FCBBF3009069 | 0 | 0 | 0 | 0 | 0 | 8.273 | 0 |
| FCBBF3009101 | 0 | 0 | 0 | 0 | 0 | 64.881 | 0 |
| FCBBF3009152 | 0 | 0 | 0 | 0 | 0 | 35.139 | 0 |
| FCBBF3009256 | 0 | 0 | 0 | 0 | 0 | 68.901 | 0 |
| FCBBF3009317 | 0 | 0 | 0 | 0 | 0 | 100 | 0 |
| FCBBF3009428 | 0 | 0 | 0 | 0 | 0 | 100 | 0 |
| FCBBF3009464 | 0 | 0 | 0 | 0 | 0 | 100 | 0 |
| FCBBF3009479 | 0 | 0 | 0 | 0 | 0 | 18.275 | 0 |
| FCBBF3009526 | 0 | 0 | 0 | 0 | 0 | 21.826 | 29.451 |
| FCBBF3009541 | 0 | 0 | 0 | 0 | 11.296 | 2.285 | 0 |
| FCBBF3009703 | 0 | 0 | 0 | 0 | 0 | 59.793 | 0 |
| FCBBF3009717 | 0 | 0 | 0 | 0 | 0 | 100 | 0 |
| FCBBF3009888 | 0 | 0 | 0 | 0 | 0 | 100 | 0 |
| FCBBF3009949 | 0 | 0 | 0 | 0 | 0 | 100 | 0 |
| FCBBF3009978 | 0 | 0 | 0 | 0 | 0 | 34.275 | 0 |
| FCBBF3010008 | 0 | 0 | 0 | 0 | 0 | 100 | 0 |
| FCBBF3010012 | 0 | 0 | 0 | 0 | 0 | 29.716 | 11.456 |
| FCBBF3010124 | 0 | 0 | 0 | 0 | 0 | 100 | 0 |
| FCBBF3010130 | 0 | 0 | 0 | 0 | 0 | 100 | 0 |
| FCBBF3010142 | 0 | 0 | 0 | 0 | 0 | 26.664 | 0 |
| FCBBF3010149 | 0 | 0 | 0 | 0 | 0 | 100 | 0 |
| FCBBF3010211 | 0 | 0 | 0 | 0 | 0 | 100 | 0 |
| FCBBF3010361 | 0 | 0 | 0 | 0 | 0 | 100 | 0 |
| FCBBF3010508 | 0 | 0 | 0 | 0 | 0 | 20.837 | 0 |
| FCBBF3010544 | 0 | 0 | 0 | 0 | 0 | 100 | 0 |
| FCBBF3010586 | 0 | 0 | 0 | 0 | 0 | 100 | 0 |
| FCBBF3010601 | 0 | 0 | 0 | 0 | 0 | 100 | 0 |
| FCBBF3010665 | 0 | 0 | 0 | 0 | 0 | 48.599 | 0 |
| FCBBF3010695 | 0 | 0 | 0 | 0 | 67.953 | 4.581 | 0 |
| FCBBF3010729 | 0 | 0 | 0 | 0 | 0 | 100 | 0 |
| FCBBF3010733 | 0 | 0 | 0 | 0 | 0 | 100 | 0 |
| FCBBF3011003 | 0 | 0 | 0 | 0 | 0 | 100 | 0 |
| FCBBF3011418 | 0 | 0 | 0 | 0 | 0 | 100 | 0 |
| FCBBF3011485 | 0 | 0 | 0 | 0 | 0 | 37.055 | 0 |
| FCBBF3011523 | 0 | 0 | 0 | 0 | 0 | 33.293 | 0 |
| FCBBF3011570 | 0 | 0 | 0 | 0 | 0 | 100 | 0 |
| FCBBF3011592 | 0 | 0 | 0 | 0 | 0 | 100 | 0 |
| FCBBF3011867 | 0 | 0 | 0 | 0 | 0 | 100 | 0 |
| FCBBF3011889 | 0 | 0 | 0 | 0 | 0 | 100 | 0 |
| FCBBF3012170 | 0 | 0 | 0 | 0 | 0 | 100 | 0 |
| FCBBF3012182 | 0 | 0 | 0 | 0 | 0 | 0.487 | 0 |
| FCBBF3012288 | 0 | 0 | 0 | 0 | 0 | 73.865 | 0 |
| FCBBF3012332 | 0 | 0 | 0 | 0 | 0 | 78.700 | 0 |
| FCBBF3012347 | 0 | 0 | 0 | 0 | 0 | 4.055 | 0 |
| FCBBF3012443 | 0 | 0 | 0 | 0 | 0 | 50.412 | 0 |
| FCBBF3012588 | 0 | 0 | 0 | 0 | 0 | 100 | 0 |
| FCBBF3012667 | 0 | 0 | 0 | 0 | 0 | 45.089 | 0 |
| FCBBF3012842 | 0 | 0 | 0 | 0 | 0 | 100 | 0 |
| FCBBF3012901 | 0 | 0 | 0 | 0 | 0 | 100 | 0 |
| FCBBF3013041 | 0 | 0 | 0 | 0 | 0 | 100 | 0 |
| FCBBF3013058 | 0 | 0 | 0 | 0 | 0 | 100 | 0 |
| FCBBF3013205 | 0 | 0 | 0 | 0 | 0 | 100 | 0 |
| FCBBF3013266 | 0 | 0 | 0 | 0 | 0 | 39.056 | 0 |
| FCBBF3013307 | 0 | 0 | 0 | 0 | 0 | 100 | 0 |
| FCBBF3013341 | 0 | 0 | 0 | 0 | 0 | 100 | 0 |
| FCBBF3013506 | 0 | 0 | 0 | 0 | 0 | 43.873 | 0 |
| FCBBF3013589 | 0 | 0 | 0 | 0 | 0 | 15.328 | 0 |
| FCBBF3013623 | 0 | 0 | 0 | 0 | 0 | 100 | 0 |
| FCBBF3013800 | 0 | 0 | 0 | 0 | 0 | 64.423 | 0 |
| FCBBF3013846 | 0 | 0 | 0 | 0 | 0 | 100 | 0 |
| FCBBF3014229 | 0 | 0 | 0 | 0 | 0 | 100 | 0 |
| FCBBF3014260 | 0 | 0 | 0 | 0 | 0 | 8.343 | 11.257 |
| FCBBF3014355 | 0 | 0 | 0 | 0 | 0 | 100 | 0 |
| FCBBF3014567 | 0 | 0 | 0 | 0 | 0 | 100 | 0 |
| FCBBF3014770 | 0 | 0 | 0 | 0 | 0 | 17.629 | 0 |
| FCBBF3015100 | 0 | 0 | 0 | 0 | 0 | 9.218 | 0 |
| FCBBF3015119 | 0 | 0 | 0 | 0 | 0 | 100 | 0 |
| FCBBF3015131 | 0 | 0 | 0 | 0 | 0 | 100 | 0 |
| FCBBF3015317 | 0 | 0 | 0 | 0 | 0 | 100 | 0 |
| FCBBF3015727 | 0 | 0 | 0 | 0 | 0 | 73.865 | 0 |
| FCBBF3015758 | 0 | 0 | 0 | 0 | 0 | 100 | 0 |
| FCBBF3015809 | 0 | 0 | 0 | 0 | 0 | 100 | 0 |
| FCBBF3015870 | 0 | 0 | 0 | 0 | 0 | 100 | 0 |
| FCBBF3016018 | 0 | 0 | 0 | 0 | 0 | 100 | 0 |
| FCBBF3016134 | 0 | 0 | 0 | 0 | 0 | 19.231 | 0 |
| FCBBF3016178 | 0 | 0 | 0 | 0 | 0 | 100 | 0 |
| FCBBF3016451 | 0 | 0 | 0 | 0 | 0 | 43.392 | 0 |
| FCBBF3016618 | 0 | 0 | 0 | 0 | 0 | 100 | 0 |
| FCBBF3016622 | 0 | 0 | 0 | 0 | 0 | 64.881 | 0 |
| FCBBF3016644 | 0 | 0 | 0 | 0 | 0 | 100 | 0 |
| FCBBF3016667 | 0 | 0 | 0 | 0 | 0 | 23.650 | 0 |
| FCBBF3016928 | 0 | 0 | 0 | 0 | 0 | 3.238 | 0 |
| FCBBF3016987 | 0 | 0 | 0 | 0 | 0 | 100 | 0 |
| FCBBF3017024 | 0 | 0 | 0 | 0 | 0 | 9.469 | 0 |
| FCBBF3017059 | 0 | 0 | 0 | 0 | 0 | 100 | 0 |
| FCBBF3017071 | 0 | 0 | 0 | 0 | 0 | 100 | 0 |
| FCBBF3017123 | 0 | 0 | 0 | 0 | 0 | 100 | 0 |
| FCBBF3017233 | 0 | 0 | 0 | 0 | 0 | 100 | 0 |
| FCBBF3017255 | 0 | 0 | 0 | 0 | 0 | 64.423 | 0 |
| FCBBF3017288 | 0 | 0 | 0 | 0 | 0 | 59.793 | 0 |
| FCBBF3017396 | 0 | 0 | 0 | 0 | 0 | 3.772 | 0 |
| FCBBF3017531 | 0 | 0 | 0 | 0 | 0 | 2.962 | 0 |
| FCBBF3017535 | 0 | 0 | 0 | 0 | 0 | 100 | 0 |
| FCBBF3017681 | 0 | 0 | 0 | 0 | 0 | 100 | 0 |
| FCBBF3017729 | 0 | 0 | 0 | 0 | 0 | 100 | 0 |
| FCBBF3017873 | 0 | 0 | 0 | 0 | 0 | 100 | 0 |
| FCBBF3017918 | 0 | 0 | 0 | 0 | 0 | 12.096 | 0 |
| FCBBF3017974 | 0 | 0 | 0 | 0 | 0 | 100 | 0 |
| FCBBF3018067 | 0 | 0 | 0 | 0 | 0 | 33.878 | 0 |
| FCBBF3018173 | 0 | 0 | 0 | 0 | 0 | 64.423 | 0 |
| FCBBF3018453 | 0 | 0 | 0 | 0 | 0 | 25.602 | 23.031 |
| FCBBF3018591 | 0 | 0 | 0 | 0 | 0 | 100 | 0 |
| FCBBF3018753 | 0 | 0 | 0 | 0 | 0 | 42.565 | 57.435 |
| FCBBF3018796 | 0 | 0 | 0 | 0 | 0 | 5.735 | 0 |
| FCBBF3018826 | 0 | 0 | 0 | 0 | 0 | 18.022 | 48.637 |
| FCBBF3018949 | 0 | 0 | 0 | 0 | 0 | 52.288 | 0 |
| FCBBF3019085 | 0 | 0 | 0 | 0 | 0 | 100 | 0 |
| FCBBF3019320 | 0 | 0 | 0 | 0 | 0 | 36.520 | 0 |
| FCBBF3019437 | 0 | 0 | 0 | 0 | 0 | 59.793 | 0 |
| FCBBF3019455 | 0 | 0 | 0 | 0 | 0 | 15.046 | 0 |
| FCBBF3019564 | 0 | 0 | 0 | 0 | 0 | 100 | 0 |
| FCBBF3019569 | 0 | 0 | 0 | 0 | 0 | 72.245 | 0 |
| FCBBF3019570 | 0 | 0 | 0 | 0 | 0 | 100 | 0 |
| FCBBF3019663 | 0 | 0 | 0 | 0 | 0 | 100 | 0 |
| FCBBF3019714 | 0 | 0 | 0 | 0 | 56.127 | 3.784 | 0 |
| FCBBF3019716 | 0 | 0 | 0 | 0 | 0 | 100 | 0 |
| FCBBF3019784 | 0 | 0 | 0 | 0 | 0 | 50.412 | 0 |
| FCBBF3019839 | 0 | 0 | 0 | 0 | 0 | 6.919 | 0 |
| FCBBF3019867 | 0 | 0 | 0 | 0 | 0 | 100 | 0 |
| FCBBF3019961 | 0 | 0 | 0 | 0 | 0 | 100 | 0 |
| FCBBF3020030 | 0 | 0 | 0 | 0 | 0 | 100 | 0 |
| FCBBF3020138 | 0 | 0 | 0 | 0 | 0 | 100 | 0 |
| FCBBF3020140 | 0 | 0 | 0 | 0 | 0 | 52.557 | 0 |
| FCBBF3020163 | 0 | 0 | 0 | 0 | 0 | 100 | 0 |
| FCBBF3020232 | 0 | 0 | 0 | 0 | 0 | 100 | 0 |
| FCBBF3020599 | 0 | 0 | 0 | 0 | 0 | 51.503 | 0 |
| FCBBF3021026 | 0 | 0 | 0 | 0 | 0 | 44.606 | 0 |
| FCBBF3021191 | 0 | 0 | 0 | 0 | 0 | 34.251 | 0 |
| FCBBF3021221 | 0 | 0 | 0 | 0 | 0 | 100 | 0 |
| FCBBF3021501 | 0 | 0 | 0 | 0 | 0 | 100 | 0 |
| FCBBF3021506 | 0 | 0 | 0 | 0 | 0 | 100 | 0 |
| FCBBF3021524 | 0 | 0 | 0 | 0 | 0 | 100 | 0 |
| FCBBF3021807 | 0 | 0 | 0 | 0 | 0 | 8.010 | 0 |
| FCBBF3021940 | 0 | 0 | 0 | 0 | 0 | 46.137 | 0 |
| FCBBF3021985 | 0 | 0 | 0 | 0 | 0 | 100 | 0 |
| FCBBF3022005 | 0 | 0 | 0 | 0 | 0 | 100 | 0 |
| FCBBF3022291 | 0 | 0 | 0 | 0 | 0 | 72.245 | 0 |
| FCBBF3022311 | 0 | 0 | 0 | 0 | 0 | 83.886 | 0 |
| FCBBF3022504 | 0 | 0 | 0 | 0 | 0 | 28.792 | 38.851 |
| FCBBF3022566 | 0 | 0 | 0 | 0 | 0 | 64.881 | 0 |
| FCBBF3022593 | 0 | 0 | 0 | 0 | 0 | 12.800 | 0 |
| FCBBF3022765 | 0 | 0 | 0 | 0 | 0 | 64.708 | 0 |
| FCBBF3022767 | 0 | 0 | 0 | 0 | 0 | 37.623 | 0 |
| FCBBF3022894 | 0 | 0 | 0 | 0 | 0 | 8.149 | 0 |
| FCBBF3023061 | 0 | 0 | 0 | 0 | 0 | 100 | 0 |
| FCBBF3023078 | 0 | 0 | 0 | 0 | 0 | 7.910 | 0 |
| FCBBF3023368 | 0 | 0 | 33.084 | 0 | 0 | 4.533 | 0 |
| FCBBF3023372 | 0 | 0 | 0 | 0 | 0 | 100 | 0 |
| FCBBF3023443 | 0 | 0 | 0 | 0 | 0 | 100 | 0 |
| FCBBF3023599 | 0 | 0 | 0 | 0 | 0 | 100 | 0 |
| FCBBF3023667 | 0 | 0 | 0 | 1.565 | 0 | 0.491 | 1.324 |
| FCBBF3023704 | 0 | 0 | 0 | 0 | 0 | 100 | 0 |
| FCBBF3023887 | 0 | 0 | 0 | 0 | 0 | 17.508 | 23.625 |
| FCBBF3023895 | 0 | 0 | 0 | 0 | 0 | 100 | 0 |
| FCBBF3024002 | 0 | 0 | 0 | 0 | 0 | 10.485 | 0 |
| FCBBF3024096 | 0 | 0 | 0 | 0 | 0 | 100 | 0 |
| FCBBF3024225 | 0 | 0 | 0 | 0 | 0 | 100 | 0 |
| FCBBF3024266 | 0 | 0 | 0 | 0 | 0 | 36.142 | 24.384 |
| FCBBF3024364 | 0 | 0 | 0 | 0 | 0 | 100 | 0 |
| FCBBF3024623 | 0 | 0 | 0 | 0 | 0 | 41.402 | 13.966 |
| FCBBF3024793 | 0 | 0 | 0 | 0 | 0 | 29.517 | 0 |
| FCBBF3024911 | 0 | 0 | 0 | 0 | 0 | 100 | 0 |
| FCBBF3025098 | 0 | 0 | 0 | 0 | 0 | 100 | 0 |
| FCBBF3025252 | 0 | 0 | 0 | 0 | 0 | 100 | 0 |
| FCBBF3025280 | 0 | 0 | 0 | 0 | 0 | 100 | 0 |
| FCBBF3025285 | 0 | 0 | 0 | 0 | 0 | 44.606 | 0 |
| FCBBF3025417 | 0 | 0 | 0 | 0 | 0 | 31.716 | 0 |
| FCBBF3025528 | 56.366 | 0 | 0 | 0 | 0 | 8.919 | 12.035 |
| FCBBF3025568 | 0 | 0 | 0 | 0 | 0 | 30.383 | 0 |
| FCBBF3025650 | 0 | 0 | 0 | 0 | 0 | 46.458 | 0 |
| FCBBF3025674 | 0 | 0 | 0 | 0 | 0 | 100 | 0 |
| FCBBF3025730 | 0 | 0 | 0 | 0 | 0 | 36.577 | 0 |
| FCBBF3025737 | 0 | 0 | 0 | 19.153 | 0 | 18.013 | 16.204 |
| FCBBF3025905 | 0 | 0 | 0 | 0 | 0 | 29.552 | 0 |
| FCBBF3026021 | 0 | 0 | 0 | 0 | 0 | 100 | 0 |
| FCBBF3026048 | 0 | 0 | 0 | 0 | 0 | 59.793 | 0 |
| FCBBF3026251 | 0 | 0 | 0 | 0 | 0 | 100 | 0 |
| FCBBF3026308 | 0 | 0 | 0 | 0 | 0 | 23.612 | 0 |
| FCBBF3026651 | 0 | 0 | 0 | 0 | 0 | 13.481 | 0 |
| FCBBF3026678 | 0 | 0 | 0 | 0 | 0 | 100 | 0 |
| FCBBF3026692 | 0 | 0 | 0 | 0 | 0 | 29.977 | 0 |
| FCBBF3027104 | 0 | 0 | 0 | 0 | 0 | 100 | 0 |
| FCBBF3027199 | 0 | 0 | 0 | 0 | 0 | 100 | 0 |
| FCBBF3027328 | 0 | 0 | 0 | 0 | 0 | 100 | 0 |
| FCBBF3027559 | 0 | 0 | 0 | 0 | 0 | 64.881 | 0 |
| FCBBF3027717 | 0 | 0 | 0 | 0 | 0 | 100 | 0 |
| FCBBF3027755 | 0 | 0 | 0 | 0 | 0 | 100 | 0 |
| FCBBF3027768 | 0 | 0 | 0 | 0 | 0 | 100 | 0 |
| FCBBF3027854 | 0 | 0 | 0 | 0 | 0 | 14.460 | 0 |
| FCBBF3027863 | 0 | 0 | 0 | 0 | 0 | 100 | 0 |
| FCBBF3027903 | 0 | 0 | 0 | 0 | 0 | 100 | 0 |
| FCBBF3028102 | 0 | 0 | 0 | 0 | 0 | 100 | 0 |
| FCBBF3028143 | 0 | 0 | 0 | 0 | 0 | 100 | 0 |
| FCBBF3028188 | 0 | 0 | 0 | 0 | 0 | 100 | 0 |
| FCBBF3028202 | 0 | 0 | 0 | 0 | 0 | 42.451 | 0 |
| FCBBF3028244 | 0 | 0 | 12.519 | 0 | 0 | 8.576 | 4.629 |
| FCBBF3028472 | 0 | 0 | 0 | 0 | 0 | 100 | 0 |
| FCBBF3028528 | 0 | 0 | 0 | 0 | 0 | 100 | 0 |
| FCBBF3028582 | 0 | 0 | 0 | 0 | 0 | 100 | 0 |
| FCBBF3028593 | 0 | 0 | 0 | 0 | 0 | 37.296 | 10.065 |
| FCBBF3028671 | 0 | 0 | 0 | 0 | 0 | 100 | 0 |
| FCBBF3028794 | 0 | 0 | 0 | 0 | 0 | 100 | 0 |
| FCBBF4000017 | 0 | 0 | 0 | 0 | 0 | 16.409 | 0 |
| FCBBF4000061 | 0 | 0 | 0 | 0 | 0 | 100 | 0 |
| FCBBF4000076 | 0 | 0 | 0 | 0 | 0 | 100 | 0 |
| FCBBF4000142 | 0 | 0 | 0 | 0 | 0 | 13.508 | 18.228 |
| FCBBF4000173 | 0 | 0 | 0 | 0 | 0 | 100 | 0 |
| FCBBF4000192 | 0 | 0 | 0 | 0 | 0 | 100 | 0 |
| FCBBF4000193 | 0 | 0 | 0 | 0 | 0 | 59.793 | 0 |
| FCBBF4000268 | 0 | 0 | 0 | 0 | 0 | 64.423 | 0 |
| FCBBF4000282 | 0 | 0 | 0 | 0 | 0 | 100 | 0 |
| FCBBF4000399 | 0 | 0 | 0 | 0 | 0 | 100 | 0 |
| FCBBF4000415 | 0 | 0 | 0 | 0 | 0 | 100 | 0 |
| FCBBF4000446 | 0 | 0 | 0 | 0 | 0 | 5.551 | 1.872 |
| FCBBF4000500 | 0 | 0 | 0 | 0 | 0 | 16.192 | 0 |
| FCBBF4000529 | 0 | 0 | 0 | 0 | 0 | 100 | 0 |
| FCBBF4000548 | 0 | 0 | 0 | 0 | 0 | 27.002 | 0 |
| FCBBF5000041 | 0 | 0 | 0 | 0 | 0 | 100 | 0 |
| FCBBF5000061 | 0 | 0 | 0 | 0 | 0 | 38.701 | 0 |
| FCBBF5000165 | 0 | 0 | 0 | 0 | 0 | 18.244 | 0 |
| FCBBF5000261 | 0 | 0 | 0 | 0 | 0 | 9.592 | 25.887 |
| FCBBF5000325 | 0 | 0 | 0 | 0 | 0 | 18.677 | 0 |
| FCBBF5000353 | 0 | 0 | 0 | 0 | 0 | 64.708 | 0 |
| FCBBF5000384 | 0 | 0 | 0 | 0 | 0 | 100 | 0 |
| FCBBF5000483 | 0 | 0 | 0 | 0 | 0 | 100 | 0 |
| FCBBF5000495 | 0 | 0 | 0 | 0 | 0 | 100 | 0 |
| FEBRA1000005 | 0 | 0 | 0 | 0 | 0 | 0 | 100 |
| FEBRA1000008 | 0 | 0 | 0 | 0 | 0 | 0 | 57.838 |
| FEBRA1000022 | 0 | 0 | 0 | 0 | 0 | 0 | 13.120 |
| FEBRA1000030 | 0 | 0 | 0 | 0 | 0 | 0 | 40.753 |
| FEBRA1000057 | 0 | 0 | 0 | 0 | 0 | 6.414 | 4.328 |
| FEBRA1000088 | 0 | 0 | 0 | 0 | 0 | 0 | 21.277 |
| FEBRA1000138 | 0 | 0 | 0 | 0 | 0 | 0 | 100 |
| FEBRA1000148 | 0 | 0 | 0 | 0 | 0 | 0 | 100 |
| FEBRA1000149 | 0 | 0 | 0 | 0 | 0 | 0 | 100 |
| FEBRA1000183 | 0 | 0 | 0 | 0 | 0 | 0 | 100 |
| FEBRA1000188 | 0 | 0 | 0 | 0 | 0 | 38.663 | 15.651 |
| FEBRA1000189 | 0 | 0 | 0 | 0 | 0 | 0 | 50.538 |
| FEBRA1000190 | 0 | 0 | 0 | 0 | 0 | 0 | 19.812 |
| FEBRA2000007 | 0 | 0 | 0 | 0 | 0 | 0 | 59.033 |
| FEBRA2000021 | 0 | 0 | 0 | 0 | 0 | 0 | 100 |
| FEBRA2000035 | 0 | 0 | 0 | 0 | 0 | 0 | 100 |
| FEBRA2000053 | 0 | 0 | 0 | 0 | 0 | 13.028 | 35.160 |
| FEBRA2000105 | 0 | 0 | 0 | 0 | 0 | 0 | 1.788 |
| FEBRA2000126 | 0 | 0 | 0 | 0 | 0 | 0 | 100 |
| FEBRA2000129 | 0 | 0 | 0 | 0 | 0 | 0 | 6.480 |
| FEBRA2000210 | 0 | 0 | 0 | 0 | 0 | 1.306 | 3.524 |
| FEBRA2000220 | 0 | 0 | 0 | 0 | 0 | 0 | 100 |
| FEBRA2000228 | 0 | 0 | 0 | 0 | 0 | 7.297 | 39.387 |
| FEBRA2000253 | 0 | 0 | 0 | 0 | 0 | 0 | 36.491 |
| FEBRA2000282 | 0 | 0 | 0 | 0 | 0 | 0 | 79.225 |
| FEBRA2000286 | 0 | 0 | 0 | 0 | 0 | 0 | 100 |
| FEBRA2000297 | 0 | 0 | 0 | 0 | 0 | 14.651 | 19.769 |
| FEBRA2000311 | 0 | 0 | 0 | 0 | 0 | 0 | 24.958 |
| FEBRA2000321 | 0 | 0 | 0 | 0 | 0 | 0 | 100 |
| FEBRA2000333 | 0 | 0 | 0 | 0 | 0 | 0 | 69.211 |
| FEBRA2000377 | 0 | 0 | 0 | 0 | 0 | 18.481 | 24.938 |
| FEBRA2000378 | 0 | 0 | 0 | 0 | 0 | 0 | 100 |
| FEBRA2000391 | 0 | 0 | 0 | 0 | 0 | 0 | 100 |
| FEBRA2000394 | 0 | 0 | 0 | 0 | 0 | 0 | 100 |
| FEBRA2000397 | 0 | 0 | 0 | 0 | 0 | 0 | 28.982 |
| FEBRA2000399 | 0 | 0 | 0 | 0 | 0 | 0 | 100 |
| FEBRA2000402 | 0 | 0 | 0 | 0 | 0 | 10.239 | 13.817 |
| FEBRA2000404 | 0 | 0 | 0 | 0 | 0 | 0 | 25.082 |
| FEBRA2000409 | 0 | 0 | 0 | 0 | 0 | 0 | 100 |
| FEBRA2000415 | 0 | 0 | 0 | 0 | 0 | 0 | 100 |
| FEBRA2000436 | 0 | 0 | 0 | 0 | 0 | 0 | 100 |
| FEBRA2000438 | 0 | 0 | 0 | 0 | 0 | 0 | 22.025 |
| FEBRA2000452 | 0 | 0 | 0 | 0 | 0 | 12.970 | 7.501 |
| FEBRA2000454 | 0 | 0 | 0 | 0 | 0 | 0 | 74.935 |
| FEBRA2000462 | 0 | 0 | 0 | 0 | 0 | 5.215 | 7.037 |
| FEBRA2000468 | 0 | 0 | 0 | 0 | 0 | 0 | 100 |
| FEBRA2000491 | 0 | 0 | 0 | 0 | 0 | 0 | 100 |
| FEBRA2000504 | 0 | 0 | 0 | 0 | 0 | 0 | 100 |
| FEBRA2000510 | 0 | 0 | 0 | 0 | 0 | 6.817 | 9.198 |
| FEBRA2000532 | 0 | 0 | 0 | 0 | 0 | 0 | 7.972 |
| FEBRA2000536 | 0 | 0 | 0 | 0 | 0 | 5.819 | 15.704 |
| FEBRA2000538 | 0 | 0 | 0 | 0 | 0 | 0 | 40.112 |
| FEBRA2000575 | 0 | 0 | 0 | 0 | 0 | 0 | 100 |
| FEBRA2000581 | 0 | 0 | 0 | 0 | 0 | 11.757 | 3.966 |
| FEBRA2000656 | 0 | 0 | 0 | 0 | 0 | 0 | 100 |
| FEBRA2000659 | 0 | 0 | 0 | 0 | 0 | 0 | 100 |
| FEBRA2000680 | 0 | 0 | 0 | 0 | 0 | 0 | 23.429 |
| FEBRA2000690 | 0 | 0 | 0 | 0 | 0 | 0 | 100 |
| FEBRA2000727 | 0 | 0 | 0 | 0 | 0 | 0 | 100 |
| FEBRA2000733 | 0 | 0 | 0 | 0 | 4.618 | 0 | 0.840 |
| FEBRA2000734 | 0 | 0 | 0 | 0 | 0 | 0 | 100 |
| FEBRA2000740 | 0 | 0 | 0 | 0 | 0 | 2.089 | 2.819 |
| FEBRA2000757 | 0 | 0 | 0 | 0 | 0 | 16.972 | 7.634 |
| FEBRA2000762 | 0 | 0 | 0 | 0 | 0 | 0 | 15.507 |
| FEBRA2000771 | 0 | 0 | 0 | 0 | 0 | 0 | 54.082 |
| FEBRA2000772 | 0 | 0 | 0 | 0 | 0 | 0 | 21.324 |
| FEBRA2000782 | 0 | 0 | 0 | 0 | 0 | 0 | 63.717 |
| FEBRA2000787 | 0 | 0 | 0 | 0 | 0 | 3.649 | 4.924 |
| FEBRA2000790 | 0 | 0 | 0 | 0 | 0 | 0 | 100 |
| FEBRA2000793 | 0 | 0 | 0 | 0 | 0 | 7.085 | 57.358 |
| FEBRA2000803 | 0 | 0 | 0 | 0 | 0 | 0 | 100 |
| FEBRA2000805 | 0 | 0 | 0 | 0 | 0 | 0 | 100 |
| FEBRA2000809 | 0 | 0 | 0 | 0 | 0 | 0 | 100 |
| FEBRA2000815 | 0 | 0 | 0 | 0 | 0 | 20.036 | 27.036 |
| FEBRA2000856 | 0 | 0 | 0 | 0 | 0 | 0 | 39.568 |
| FEBRA2000862 | 0 | 0 | 0 | 0 | 0 | 0 | 100 |
| FEBRA2000877 | 0 | 0 | 0 | 0 | 0 | 0 | 35.466 |
| FEBRA2000880 | 0 | 0 | 0 | 0 | 0 | 0 | 26.417 |
| FEBRA2000881 | 0 | 0 | 0 | 0 | 0 | 0 | 7.891 |
| FEBRA2000901 | 0 | 0 | 0 | 0 | 0 | 0 | 25.894 |
| FEBRA2000909 | 0 | 0 | 0 | 0 | 0 | 0 | 100 |
| FEBRA2000917 | 0 | 0 | 0 | 0 | 0 | 0 | 100 |
| FEBRA2000928 | 0 | 0 | 0 | 0 | 0 | 0 | 100 |
| FEBRA2000936 | 0 | 0 | 0 | 0 | 0 | 0 | 100 |
| FEBRA2000937 | 0 | 0 | 0 | 0 | 0 | 0 | 100 |
| FEBRA2000959 | 0 | 0 | 0 | 0 | 0 | 0 | 100 |
| FEBRA2000972 | 0 | 0 | 0 | 0 | 0 | 0 | 37.761 |
| FEBRA2001004 | 0 | 0 | 0 | 0 | 0 | 0 | 55.143 |
| FEBRA2001012 | 0 | 0 | 0 | 0 | 0 | 0 | 100 |
| FEBRA2001020 | 0 | 0 | 0 | 0 | 0 | 0 | 100 |
| FEBRA2001093 | 0 | 0 | 0 | 0 | 0 | 0 | 100 |
| FEBRA2001124 | 0 | 0 | 0 | 0 | 0 | 0 | 100 |
| FEBRA2001133 | 0 | 0 | 0 | 0 | 0 | 0 | 79.225 |
| FEBRA2001146 | 0 | 0 | 0 | 0 | 0 | 0 | 27.877 |
| FEBRA2001161 | 0 | 0 | 0 | 0 | 0 | 0 | 100 |
| FEBRA2001175 | 0 | 0 | 0 | 0 | 0 | 0 | 100 |
| FEBRA2001197 | 0 | 0 | 0 | 0 | 0 | 0 | 35.141 |
| FEBRA2001217 | 0 | 0 | 0 | 0 | 0 | 0 | 100 |
| FEBRA2001227 | 0 | 0 | 0 | 0 | 0 | 13.661 | 27.650 |
| FEBRA2001245 | 0 | 0 | 0 | 0 | 0 | 9.320 | 6.288 |
| FEBRA2001267 | 0 | 0 | 0 | 0 | 0 | 0 | 100 |
| FEBRA2001294 | 28.213 | 0 | 0 | 14.242 | 0 | 0 | 6.024 |
| FEBRA2001334 | 0 | 0 | 0 | 0 | 0 | 10.624 | 14.336 |
| FEBRA2001351 | 0 | 0 | 0 | 0 | 0 | 0 | 100 |
| FEBRA2001383 | 0 | 0 | 0 | 0 | 0 | 19.272 | 26.005 |
| FEBRA2001438 | 0 | 0 | 0 | 0 | 0 | 0 | 100 |
| FEBRA2001440 | 0 | 0 | 0 | 0 | 0 | 0 | 58.031 |
| FEBRA2001487 | 0 | 0 | 0 | 0 | 0 | 0 | 100 |
| FEBRA2001492 | 0 | 0 | 0 | 0 | 0 | 5.979 | 8.068 |
| FEBRA2001523 | 0 | 0 | 0 | 0 | 0 | 0 | 21.196 |
| FEBRA2001561 | 0 | 0 | 0 | 0 | 0 | 0 | 100 |
| FEBRA2001571 | 0 | 0 | 0 | 0 | 0 | 3.874 | 20.911 |
| FEBRA2001581 | 0 | 0 | 0 | 0 | 0 | 7.575 | 20.443 |
| FEBRA2001584 | 0 | 0 | 0 | 0 | 0 | 0 | 100 |
| FEBRA2001590 | 0 | 0 | 0 | 0 | 0 | 0 | 100 |
| FEBRA2001591 | 0 | 0 | 0 | 0 | 0 | 37.743 | 50.929 |
| FEBRA2001610 | 0 | 0 | 0 | 0 | 0 | 0 | 100 |
| FEBRA2001620 | 0 | 0 | 0 | 0 | 0 | 0 | 100 |
| FEBRA2001659 | 0 | 0 | 0 | 0 | 0 | 0 | 100 |
| FEBRA2001669 | 0 | 0 | 0 | 0 | 0 | 5.937 | 13.353 |
| FEBRA2001698 | 0 | 0 | 0 | 0 | 0 | 0 | 30.346 |
| FEBRA2001705 | 0 | 0 | 0 | 0 | 0 | 0 | 100 |
| FEBRA2001706 | 0 | 0 | 0 | 0 | 0 | 0 | 71.370 |
| FEBRA2001715 | 0 | 0 | 0 | 0 | 0 | 1.842 | 2.486 |
| FEBRA2001745 | 0 | 0 | 0 | 0 | 0 | 6.946 | 6.248 |
| FEBRA2001752 | 0 | 0 | 0 | 0 | 0 | 0 | 78.526 |
| FEBRA2001772 | 0 | 0 | 0 | 0 | 0 | 0 | 100 |
| FEBRA2001790 | 0 | 0 | 0 | 0 | 0 | 0 | 100 |
| FEBRA2001814 | 0 | 0 | 0 | 0 | 0 | 2.444 | 16.490 |
| FEBRA2001828 | 0 | 0 | 0 | 0 | 0 | 0 | 27.125 |
| FEBRA2001867 | 0 | 0 | 0 | 0 | 0 | 13.694 | 18.478 |
| FEBRA2001914 | 0 | 0 | 0 | 0 | 0 | 0 | 100 |
| FEBRA2001974 | 0 | 0 | 0 | 0 | 0 | 0 | 29.046 |
| FEBRA2001989 | 0 | 0 | 0 | 0 | 0 | 0 | 74.935 |
| FEBRA2001990 | 0 | 0 | 0 | 0 | 0 | 0 | 9.046 |
| FEBRA2002009 | 0 | 0 | 0 | 0 | 0 | 0 | 100 |
| FEBRA2002027 | 0 | 0 | 0 | 0 | 0 | 0 | 44.210 |
| FEBRA2002086 | 0 | 0 | 0 | 0 | 0 | 0 | 100 |
| FEBRA2002109 | 0 | 0 | 0 | 0 | 0 | 0 | 100 |
| FEBRA2002191 | 68.584 | 0 | 0 | 0 | 0 | 10.853 | 14.644 |
| FEBRA2002260 | 0 | 0 | 0 | 0 | 0 | 0 | 100 |
| FEBRA2002352 | 0 | 0 | 0 | 0 | 0 | 0 | 17.307 |
| FEBRA2002399 | 0 | 0 | 0 | 0 | 0 | 0 | 100 |
| FEBRA2002410 | 0 | 0 | 0 | 0 | 0 | 0 | 66.741 |
| FEBRA2002429 | 0 | 0 | 0 | 0 | 0 | 0 | 9.761 |
| FEBRA2002442 | 0 | 0 | 0 | 0 | 0 | 0 | 52.075 |
| FEBRA2002456 | 0 | 0 | 0 | 0 | 0 | 18.135 | 8.157 |
| FEBRA2002508 | 0 | 0 | 0 | 0 | 0 | 0 | 100 |
| FEBRA2002525 | 0 | 0 | 0 | 0 | 0 | 0 | 66.741 |
| FEBRA2002527 | 0 | 0 | 0 | 0 | 0 | 0 | 7.139 |
| FEBRA2002552 | 0 | 0 | 0 | 0 | 0 | 0 | 100 |
| FEBRA2002574 | 0 | 0 | 0 | 0 | 0 | 0 | 74.935 |
| FEBRA2002601 | 0 | 0 | 0 | 0 | 0 | 0 | 26.611 |
| FEBRA2002611 | 0 | 0 | 0 | 0 | 0 | 0 | 54.171 |
| FEBRA2002628 | 0 | 0 | 0 | 0 | 0 | 0 | 100 |
| FEBRA2002662 | 0 | 0 | 0 | 0 | 0 | 0 | 100 |
| FEBRA2002682 | 0 | 0 | 0 | 0 | 0 | 0 | 66.741 |
| FEBRA2002707 | 0 | 0 | 0 | 0 | 0 | 6.657 | 8.982 |
| FEBRA2002727 | 0 | 0 | 0 | 0 | 0 | 0 | 100 |
| FEBRA2002781 | 0 | 0 | 0 | 0 | 0 | 0 | 17.057 |
| FEBRA2002783 | 0 | 0 | 0 | 0 | 0 | 0 | 100 |
| FEBRA2002792 | 0 | 0 | 0 | 0 | 0 | 0 | 6.673 |
| FEBRA2002818 | 0 | 0 | 0 | 0 | 0 | 0 | 100 |
| FEBRA2002858 | 0 | 0 | 0 | 0 | 0 | 0 | 100 |
| FEBRA2002882 | 0 | 0 | 0 | 0 | 0 | 0 | 100 |
| FEBRA2002897 | 0 | 0 | 0 | 0 | 0 | 0 | 100 |
| FEBRA2002908 | 0 | 0 | 0 | 0 | 0 | 0 | 100 |
| FEBRA2002931 | 0 | 0 | 0 | 0 | 0 | 0 | 5.456 |
| FEBRA2002933 | 0 | 0 | 0 | 0 | 0 | 0.684 | 0.923 |
| FEBRA2002962 | 0 | 0 | 0 | 0 | 0 | 0 | 100 |
| FEBRA2002986 | 0 | 0 | 0 | 0 | 0 | 0 | 100 |
| FEBRA2003054 | 0 | 0 | 0 | 0 | 0 | 0 | 100 |
| FEBRA2003100 | 0 | 0 | 0 | 0 | 0 | 4.867 | 6.567 |
| FEBRA2003115 | 0 | 0 | 0 | 0 | 0 | 0 | 54.040 |
| FEBRA2003128 | 0 | 0 | 0 | 0 | 0 | 0 | 14.503 |
| FEBRA2003155 | 0 | 0 | 0 | 0 | 0 | 0 | 100 |
| FEBRA2003163 | 0 | 0 | 0 | 0 | 0 | 0 | 100 |
| FEBRA2003181 | 0 | 0 | 0 | 0 | 0 | 0 | 74.935 |
| FEBRA2003198 | 0 | 0 | 0 | 0 | 0 | 0 | 66.741 |
| FEBRA2003253 | 0 | 0 | 0 | 0 | 0 | 0 | 100 |
| FEBRA2003258 | 0 | 0 | 0 | 0 | 0 | 5.227 | 21.158 |
| FEBRA2003275 | 0 | 0 | 0 | 0 | 0 | 0 | 100 |
| FEBRA2003308 | 0 | 0 | 0 | 0 | 0 | 0 | 100 |
| FEBRA2003327 | 0 | 0 | 0 | 0 | 0 | 0 | 100 |
| FEBRA2003429 | 0 | 0 | 0 | 0 | 0 | 0 | 100 |
| FEBRA2003436 | 0 | 0 | 0 | 0 | 0 | 10.270 | 13.858 |
| FEBRA2003454 | 0 | 0 | 0 | 0 | 0 | 0 | 100 |
| FEBRA2003468 | 0 | 0 | 0 | 0 | 0 | 0 | 100 |
| FEBRA2003470 | 0 | 0 | 0 | 0 | 0 | 0 | 36.383 |
| FEBRA2003520 | 0 | 0 | 0 | 0 | 0 | 0 | 100 |
| FEBRA2003524 | 0 | 0 | 0 | 0 | 0 | 0 | 25.471 |
| FEBRA2003541 | 0 | 0 | 0 | 0 | 0 | 0 | 26.627 |
| FEBRA2003675 | 0 | 0 | 0 | 0 | 0 | 0 | 100 |
| FEBRA2003707 | 0 | 0 | 0 | 0 | 0 | 0 | 52.075 |
| FEBRA2003711 | 0 | 0 | 0 | 0 | 0 | 0 | 100 |
| FEBRA2003726 | 0 | 0 | 0 | 0 | 0 | 0 | 2.482 |
| FEBRA2003750 | 0 | 0 | 0 | 0 | 0 | 0 | 9.102 |
| FEBRA2003822 | 0 | 0 | 0 | 0 | 0 | 0 | 13.711 |
| FEBRA2003833 | 0 | 0 | 0 | 0 | 0 | 0 | 8.621 |
| FEBRA2003897 | 0 | 0 | 0 | 0 | 0 | 0 | 100 |
| FEBRA2003907 | 0 | 0 | 0 | 0 | 0 | 0 | 77.838 |
| FEBRA2003926 | 0 | 0 | 0 | 0 | 0 | 0 | 1.740 |
| FEBRA2003930 | 0 | 0 | 0 | 0 | 0 | 3.320 | 13.441 |
| FEBRA2004023 | 0 | 0 | 0 | 0 | 0 | 0 | 79.225 |
| FEBRA2004026 | 0 | 0 | 0 | 0 | 0 | 0 | 100 |
| FEBRA2004029 | 0 | 0 | 0 | 0 | 0 | 0 | 100 |
| FEBRA2004042 | 0 | 0 | 0 | 0 | 0 | 7.838 | 5.288 |
| FEBRA2004053 | 0 | 0 | 0 | 0 | 0 | 0 | 100 |
| FEBRA2004056 | 0 | 0 | 0 | 0 | 0 | 0 | 100 |
| FEBRA2004091 | 0 | 0 | 0 | 0 | 0 | 0 | 26.345 |
| FEBRA2004110 | 0 | 0 | 0 | 0 | 0 | 0 | 66.741 |
| FEBRA2004191 | 0 | 0 | 0 | 0 | 0 | 0 | 49.437 |
| FEBRA2004202 | 0 | 0 | 0 | 0 | 0 | 0 | 100 |
| FEBRA2004219 | 0 | 0 | 0 | 0 | 0 | 0 | 70.959 |
| FEBRA2004224 | 0 | 0 | 0 | 0 | 0 | 0 | 2.886 |
| FEBRA2004237 | 0 | 0 | 0 | 0 | 0 | 0 | 46.754 |
| FEBRA2004268 | 0 | 0 | 0 | 0 | 0 | 0 | 87.538 |
| FEBRA2004293 | 0 | 0 | 0 | 0 | 0 | 4.911 | 13.254 |
| FEBRA2004325 | 0 | 0 | 0 | 0 | 0 | 6.507 | 8.780 |
| FEBRA2004329 | 0 | 0 | 0 | 0 | 0 | 0 | 100 |
| FEBRA2004331 | 0 | 0 | 0 | 0 | 0 | 0 | 74.935 |
| FEBRA2004412 | 0 | 0 | 0 | 0 | 0 | 27.120 | 14.638 |
| FEBRA2004443 | 0 | 0 | 0 | 0 | 0 | 0 | 100 |
| FEBRA2004485 | 0 | 0 | 0 | 0 | 0 | 0 | 17.301 |
| FEBRA2004490 | 0 | 0 | 0 | 0 | 0 | 0 | 70.274 |
| FEBRA2004538 | 0 | 0 | 0 | 0 | 0 | 0 | 100 |
| FEBRA2004592 | 0 | 0 | 0 | 0 | 0 | 0 | 40.112 |
| FEBRA2004620 | 0 | 0 | 0 | 0 | 0 | 0 | 24.429 |
| FEBRA2004628 | 0 | 0 | 0 | 0 | 0 | 0 | 7.111 |
| FEBRA2004651 | 0 | 0 | 0 | 0 | 0 | 0 | 100 |
| FEBRA2004767 | 0 | 0 | 0 | 0 | 0 | 0 | 63.717 |
| FEBRA2004813 | 0 | 0 | 0 | 0 | 0 | 0 | 100 |
| FEBRA2004818 | 0 | 0 | 0 | 0 | 0 | 2.516 | 3.395 |
| FEBRA2004840 | 0 | 0 | 0 | 0 | 0 | 0 | 100 |
| FEBRA2004852 | 0 | 0 | 0 | 0 | 0 | 0 | 67.639 |
| FEBRA2004867 | 0 | 0 | 0 | 0 | 0 | 0 | 100 |
| FEBRA2004914 | 0 | 0 | 0 | 0 | 0 | 0 | 100 |
| FEBRA2004933 | 0 | 0 | 0 | 0 | 0 | 0 | 100 |
| FEBRA2004987 | 0 | 0 | 0 | 0 | 0 | 0 | 100 |
| FEBRA2005014 | 0 | 0 | 0 | 0 | 0 | 0 | 100 |
| FEBRA2005036 | 0 | 0 | 0 | 0 | 0 | 0 | 100 |
| FEBRA2005079 | 0 | 0 | 0 | 0 | 0 | 0 | 83.188 |
| FEBRA2005114 | 0 | 0 | 0 | 0 | 0 | 12.397 | 16.727 |
| FEBRA2005216 | 0 | 0 | 0 | 0 | 0 | 0 | 100 |
| FEBRA2005291 | 0 | 0 | 0 | 0 | 0 | 0 | 9.040 |
| FEBRA2005361 | 0 | 0 | 0 | 0 | 0 | 5.176 | 17.460 |
| FEBRA2005377 | 0 | 0 | 0 | 0 | 0 | 0 | 100 |
| FEBRA2005380 | 0 | 0 | 0 | 0 | 0 | 0 | 100 |
| FEBRA2005416 | 0 | 0 | 0 | 0 | 29.427 | 0 | 2.677 |
| FEBRA2005427 | 0 | 0 | 0 | 0 | 0 | 0 | 71.370 |
| FEBRA2005458 | 0 | 0 | 0 | 0 | 0 | 0 | 100 |
| FEBRA2005476 | 0 | 0 | 0 | 0 | 0 | 0 | 12.986 |
| FEBRA2005572 | 0 | 0 | 0 | 0 | 0 | 3.292 | 4.442 |
| FEBRA2005701 | 0 | 0 | 0 | 0 | 0 | 0 | 100 |
| FEBRA2005726 | 0 | 0 | 0 | 0 | 0 | 0 | 100 |
| FEBRA2005734 | 0 | 0 | 0 | 0 | 23.137 | 0 | 2.105 |
| FEBRA2005752 | 0 | 0 | 0 | 0 | 0 | 0 | 10.376 |
| FEBRA2005778 | 0 | 0 | 0 | 0 | 0 | 11.083 | 14.955 |
| FEBRA2005787 | 0 | 0 | 0 | 0 | 0 | 0 | 74.935 |
| FEBRA2005788 | 0 | 0 | 0 | 0 | 0 | 18.236 | 12.303 |
| FEBRA2005824 | 0 | 0 | 0 | 0 | 0 | 1.378 | 0.930 |
| FEBRA2005995 | 0 | 0 | 0 | 0 | 0 | 12.949 | 10.484 |
| FEBRA2005998 | 0 | 0 | 0 | 0 | 0 | 0 | 100 |
| FEBRA2006055 | 0 | 0 | 0 | 0 | 0 | 0 | 13.169 |
| FEBRA2006061 | 0 | 0 | 0 | 0 | 0 | 0 | 100 |
| FEBRA2006092 | 0 | 0 | 0 | 3.664 | 0 | 4.595 | 3.100 |
| FEBRA2006112 | 0 | 0 | 0 | 0 | 0 | 0 | 16.454 |
| FEBRA2006150 | 0 | 0 | 0 | 0 | 0 | 0 | 100 |
| FEBRA2006165 | 0 | 0 | 0 | 0 | 0 | 0 | 100 |
| FEBRA2006255 | 0 | 0 | 0 | 0 | 0 | 14.444 | 12.993 |
| FEBRA2006270 | 0 | 0 | 15.763 | 13.779 | 0 | 0 | 2.914 |
| FEBRA2006315 | 0 | 0 | 0 | 0 | 0 | 0 | 100 |
| FEBRA2006346 | 0 | 0 | 0 | 0 | 0 | 0 | 37.034 |
| FEBRA2006354 | 0 | 0 | 0 | 0 | 0 | 0 | 100 |
| FEBRA2006357 | 0 | 0 | 0 | 0 | 0 | 2.447 | 1.651 |
| FEBRA2006368 | 0 | 0 | 0 | 0 | 0 | 11.481 | 15.492 |
| FEBRA2006370 | 0 | 0 | 0 | 0 | 0 | 0 | 35.615 |
| FEBRA2006372 | 0 | 0 | 0 | 0 | 0 | 0 | 13.392 |
| FEBRA2006379 | 0 | 0 | 0 | 0 | 0 | 0 | 100 |
| FEBRA2006396 | 0 | 0 | 0 | 0 | 0 | 29.698 | 20.037 |
| FEBRA2006401 | 0 | 0 | 0 | 0 | 0 | 1.391 | 1.251 |
| FEBRA2006409 | 0 | 0 | 0 | 0 | 0 | 0 | 100 |
| FEBRA2006418 | 0 | 0 | 0 | 0 | 0 | 0 | 100 |
| FEBRA2006427 | 0 | 0 | 0 | 0 | 0 | 0 | 100 |
| FEBRA2006476 | 0 | 0 | 0 | 0 | 0 | 0 | 100 |
| FEBRA2006485 | 0 | 0 | 0 | 0 | 0 | 5.620 | 7.583 |
| FEBRA2006491 | 0 | 0 | 0 | 0 | 0 | 0 | 37.019 |
| FEBRA2006519 | 0 | 0 | 0 | 0 | 0 | 0 | 29.644 |
| FEBRA2006521 | 0 | 0 | 0 | 0 | 0 | 28.259 | 19.066 |
| FEBRA2006553 | 0 | 0 | 0 | 0 | 0 | 21.927 | 9.862 |
| FEBRA2006627 | 0 | 0 | 0 | 0 | 0 | 38.121 | 25.719 |
| FEBRA2006664 | 0 | 0 | 0 | 0 | 0 | 0 | 79.225 |
| FEBRA2006667 | 0 | 0 | 0 | 0 | 0 | 0 | 100 |
| FEBRA2006726 | 0 | 0 | 0 | 0 | 0 | 0 | 100 |
| FEBRA2006736 | 0 | 0 | 0 | 0 | 0 | 0 | 37.034 |
| FEBRA2006793 | 0 | 0 | 0 | 0 | 0 | 0 | 100 |
| FEBRA2006808 | 0 | 0 | 0 | 0 | 0 | 7.584 | 40.932 |
| FEBRA2006817 | 0 | 0 | 0 | 0 | 0 | 0 | 40.876 |
| FEBRA2006873 | 0 | 0 | 0 | 0 | 0 | 0 | 11.194 |
| FEBRA2006890 | 0 | 0 | 0 | 0 | 0 | 0 | 55.485 |
| FEBRA2006942 | 0 | 0 | 0 | 0 | 0 | 0 | 100 |
| FEBRA2006977 | 0 | 0 | 0 | 0 | 0 | 0 | 100 |
| FEBRA2007017 | 0 | 0 | 0 | 0 | 0 | 0 | 40.825 |
| FEBRA2007176 | 0 | 0 | 0 | 0 | 0 | 0 | 71.370 |
| FEBRA2007200 | 0 | 0 | 0 | 0 | 0 | 0 | 79.225 |
| FEBRA2007212 | 0 | 0 | 0 | 0 | 0 | 0 | 9.786 |
| FEBRA2007247 | 0 | 0 | 0 | 70.274 | 0 | 0 | 29.726 |
| FEBRA2007280 | 0 | 0 | 0 | 0 | 0 | 0 | 100 |
| FEBRA2007414 | 0 | 0 | 0 | 0 | 0 | 0 | 100 |
| FEBRA2007436 | 0 | 0 | 0 | 0 | 0 | 0 | 100 |
| FEBRA2007449 | 0 | 0 | 0 | 0 | 0 | 0 | 100 |
| FEBRA2007458 | 0 | 0 | 0 | 0 | 0 | 15.061 | 20.323 |
| FEBRA2007534 | 0 | 0 | 0 | 0 | 0 | 0 | 44.990 |
| FEBRA2007544 | 0 | 0 | 0 | 0 | 0 | 4.523 | 12.206 |
| FEBRA2007551 | 0 | 0 | 0 | 0 | 0 | 0 | 69.211 |
| FEBRA2007566 | 0 | 0 | 0 | 0 | 0 | 0 | 100 |
| FEBRA2007589 | 0 | 0 | 0 | 0 | 0 | 0 | 17.044 |
| FEBRA2007613 | 0 | 0 | 0 | 0 | 0 | 12.261 | 8.272 |
| FEBRA2007620 | 0 | 0 | 0 | 0 | 0 | 0 | 79.225 |
| FEBRA2007622 | 0 | 0 | 0 | 0 | 28.270 | 3.812 | 2.572 |
| FEBRA2007708 | 0 | 0 | 0 | 0 | 0 | 0 | 21.306 |
| FEBRA2007714 | 0 | 0 | 0 | 0 | 0 | 15.415 | 20.800 |
| FEBRA2007793 | 0 | 0 | 0 | 0 | 0 | 0 | 50.083 |
| FEBRA2007801 | 0 | 0 | 0 | 0 | 0 | 4.561 | 6.154 |
| FEBRA2007818 | 0 | 0 | 0 | 0 | 0 | 0 | 13.940 |
| FEBRA2007823 | 0 | 0 | 0 | 0 | 0 | 0 | 71.370 |
| FEBRA2007880 | 0 | 0 | 0 | 0 | 0 | 0 | 18.765 |
| FEBRA2007900 | 0 | 0 | 0 | 0 | 0 | 0 | 19.322 |
| FEBRA2007901 | 0 | 0 | 49.976 | 0 | 0 | 0 | 9.239 |
| FEBRA2007931 | 0 | 0 | 0 | 0 | 0 | 0 | 100 |
| FEBRA2007957 | 0 | 0 | 0 | 0 | 0 | 0 | 45.384 |
| FEBRA2008009 | 0 | 0 | 0 | 0 | 0 | 0 | 100 |
| FEBRA2008036 | 0 | 0 | 0 | 0 | 0 | 2.639 | 24.924 |
| FEBRA2008081 | 0 | 0 | 0 | 0 | 0 | 1.308 | 3.531 |
| FEBRA2008086 | 0 | 0 | 0 | 0 | 0 | 0 | 69.211 |
| FEBRA2008087 | 2.093 | 0 | 0 | 0 | 0 | 0 | 1.341 |
| FEBRA2008134 | 0 | 0 | 0 | 0 | 0 | 0 | 71.215 |
| FEBRA2008135 | 0 | 0 | 0 | 0 | 0 | 0 | 100 |
| FEBRA2008159 | 0 | 0 | 0 | 0 | 0 | 0 | 77.838 |
| FEBRA2008165 | 0 | 0 | 0 | 0 | 0 | 0 | 100 |
| FEBRA2008172 | 0 | 0 | 0 | 0 | 0 | 0 | 100 |
| FEBRA2008201 | 0 | 0 | 0 | 0 | 0 | 0 | 5.978 |
| FEBRA2008210 | 0 | 0 | 0 | 0 | 0 | 0 | 16.720 |
| FEBRA2008255 | 0 | 0 | 0 | 0 | 0 | 0 | 12.615 |
| FEBRA2008265 | 0 | 0 | 0 | 0 | 0 | 0 | 40.825 |
| FEBRA2008266 | 0 | 0 | 0 | 0 | 0 | 0 | 22.652 |
| FEBRA2008282 | 0 | 0 | 0 | 0 | 0 | 0 | 100 |
| FEBRA2008287 | 0 | 0 | 0 | 0 | 0 | 0 | 7.348 |
| FEBRA2008302 | 12.424 | 0 | 0 | 0 | 0 | 0 | 5.306 |
| FEBRA2008311 | 0 | 0 | 0 | 0 | 0 | 0 | 100 |
| FEBRA2008341 | 0 | 0 | 0 | 0 | 0 | 0 | 16.375 |
| FEBRA2008360 | 0 | 0 | 0 | 0 | 0 | 37.775 | 25.486 |
| FEBRA2008459 | 0 | 0 | 0 | 0 | 0 | 0 | 71.215 |
| FEBRA2008468 | 0 | 0 | 0 | 0 | 0 | 0 | 100 |
| FEBRA2008475 | 0 | 0 | 0 | 0 | 0 | 0 | 42.067 |
| FEBRA2008481 | 0 | 0 | 0 | 0 | 0 | 0 | 100 |
| FEBRA2008542 | 0 | 0 | 0 | 0 | 0 | 0 | 100 |
| FEBRA2008583 | 0 | 0 | 0 | 0.779 | 0 | 5.128 | 5.931 |
| FEBRA2008660 | 0 | 0 | 0 | 0 | 0 | 0 | 100 |
| FEBRA2008662 | 0 | 0 | 0 | 0 | 0 | 5.865 | 11.870 |
| FEBRA2008681 | 0 | 0 | 0 | 0 | 0 | 0 | 100 |
| FEBRA2008692 | 0 | 0 | 0 | 0 | 0 | 0 | 6.116 |
| FEBRA2008741 | 0 | 0 | 0 | 0 | 0 | 0 | 100 |
| FEBRA2008755 | 0 | 0 | 0 | 0 | 0 | 0 | 4.095 |
| FEBRA2008836 | 0 | 0 | 0 | 0 | 0 | 0 | 79.225 |
| FEBRA2008859 | 0 | 0 | 0 | 0 | 0 | 0 | 77.838 |
| FEBRA2008861 | 0 | 0 | 0 | 0 | 0 | 10.794 | 14.565 |
| FEBRA2008866 | 0 | 0 | 0 | 0 | 0 | 0 | 100 |
| FEBRA2008881 | 0 | 0 | 0 | 0 | 0 | 0 | 100 |
| FEBRA2008928 | 0 | 0 | 0 | 0 | 0 | 0 | 77.838 |
| FEBRA2008983 | 0 | 0 | 0 | 0 | 0 | 0 | 100 |
| FEBRA2009016 | 0 | 0 | 0 | 0 | 0 | 0 | 100 |
| FEBRA2009022 | 0 | 0 | 0 | 0 | 0 | 0 | 8.325 |
| FEBRA2009029 | 0 | 0 | 0 | 0 | 0 | 0 | 100 |
| FEBRA2009074 | 0 | 0 | 0 | 0 | 0 | 0 | 100 |
| FEBRA2009117 | 0 | 0 | 0 | 0 | 0 | 0 | 100 |
| FEBRA2009120 | 0 | 0 | 0 | 0 | 0 | 0 | 100 |
| FEBRA2009162 | 0 | 0 | 0 | 0 | 0 | 0 | 100 |
| FEBRA2009185 | 0 | 0 | 0 | 0 | 0 | 0 | 11.324 |
| FEBRA2009240 | 0 | 0 | 0 | 0 | 0 | 0 | 100 |
| FEBRA2009265 | 0 | 0 | 0 | 0 | 0 | 0 | 100 |
| FEBRA2009276 | 0 | 0 | 0 | 0 | 0 | 0 | 58.346 |
| FEBRA2009289 | 0 | 0 | 0 | 0 | 0 | 0 | 34.980 |
| FEBRA2009327 | 0 | 0 | 0 | 0 | 0 | 0 | 100 |
| FEBRA2009328 | 0 | 0 | 0 | 0 | 0 | 0 | 12.353 |
| FEBRA2009352 | 0 | 0 | 0 | 0 | 0 | 0 | 70.959 |
| FEBRA2009362 | 0 | 0 | 0 | 0 | 0 | 0 | 37.960 |
| FEBRA2009385 | 0 | 0 | 0 | 0 | 0 | 0 | 100 |
| FEBRA2009416 | 0 | 0 | 0 | 0 | 0 | 0 | 100 |
| FEBRA2009419 | 0 | 0 | 0 | 0 | 0 | 4.882 | 3.294 |
| FEBRA2009437 | 0 | 0 | 0 | 0 | 0 | 0 | 9.084 |
| FEBRA2009478 | 0 | 0 | 0 | 0 | 0 | 0 | 100 |
| FEBRA2009507 | 0 | 0 | 0 | 0 | 0 | 0 | 100 |
| FEBRA2009514 | 0 | 0 | 0 | 0 | 0 | 12.239 | 24.772 |
| FEBRA2009541 | 0 | 0 | 0 | 0 | 0 | 0 | 41.464 |
| FEBRA2009567 | 0 | 0 | 0 | 0 | 0 | 0 | 100 |
| FEBRA2009588 | 0 | 0 | 0 | 0 | 0 | 8.952 | 24.158 |
| FEBRA2009731 | 0 | 0 | 0 | 0 | 0 | 0 | 100 |
| FEBRA2009754 | 0 | 0 | 0 | 0 | 0 | 0 | 100 |
| FEBRA2009780 | 0 | 0 | 0 | 0 | 0 | 0 | 68.997 |
| FEBRA2009846 | 0 | 0 | 0 | 0 | 0 | 0 | 51.462 |
| FEBRA2009986 | 0 | 0 | 0 | 0 | 0 | 11.874 | 16.023 |
| FEBRA2010141 | 0 | 0 | 0 | 0 | 0 | 0 | 100 |
| FEBRA2010299 | 0 | 0 | 0 | 0 | 0 | 0 | 100 |
| FEBRA2010484 | 0 | 0 | 0 | 0 | 0 | 0 | 100 |
| FEBRA2010719 | 0 | 0 | 0 | 0 | 0 | 0 | 43.257 |
| FEBRA2010746 | 0 | 0 | 0 | 0 | 0 | 0 | 21.386 |
| FEBRA2010768 | 0 | 0 | 0 | 0 | 0 | 0 | 40.876 |
| FEBRA2010802 | 0 | 0 | 0 | 0 | 0 | 0 | 100 |
| FEBRA2011063 | 0 | 0 | 0 | 0 | 0 | 0 | 100 |
| FEBRA2011090 | 0 | 0 | 0 | 0 | 0 | 0 | 43.266 |
| FEBRA2011356 | 0 | 0 | 0 | 0 | 0 | 0 | 100 |
| FEBRA2011392 | 0 | 0 | 0 | 0 | 0 | 0 | 67.639 |
| FEBRA2011414 | 0 | 0 | 0 | 0 | 0 | 0 | 100 |
| FEBRA2011593 | 0 | 0 | 0 | 0 | 0 | 0 | 100 |
| FEBRA2011665 | 0 | 0 | 0 | 0 | 0 | 0 | 100 |
| FEBRA2012090 | 0 | 0 | 0 | 0 | 0 | 0 | 100 |
| FEBRA2012120 | 0 | 0 | 0 | 0 | 0 | 0 | 40.243 |
| FEBRA2012181 | 0 | 0 | 0 | 0 | 0 | 0 | 41.159 |
| FEBRA2012195 | 0 | 0 | 0 | 0 | 0 | 0 | 100 |
| FEBRA2012397 | 0 | 0 | 0 | 0 | 0 | 0 | 58.346 |
| FEBRA2012418 | 0 | 0 | 0 | 0 | 0 | 0 | 100 |
| FEBRA2012507 | 0 | 0 | 0 | 0 | 0 | 0 | 18.603 |
| FEBRA2012625 | 0 | 0 | 0 | 0 | 0 | 0 | 100 |
| FEBRA2013019 | 0 | 0 | 0 | 0 | 0 | 42.565 | 57.435 |
| FEBRA2013069 | 0 | 0 | 0 | 0 | 0 | 0 | 100 |
| FEBRA2013274 | 0 | 0 | 0 | 0 | 0 | 0 | 100 |
| FEBRA2013465 | 0 | 0 | 0 | 0 | 0 | 0 | 100 |
| FEBRA2013570 | 0 | 0 | 0 | 0 | 0 | 0 | 27.839 |
| FEBRA2013699 | 0 | 0 | 0 | 0 | 0 | 0 | 17.841 |
| FEBRA2013905 | 0 | 0 | 0 | 0 | 0 | 0 | 100 |
| FEBRA2013951 | 0 | 0 | 0 | 0 | 0 | 0 | 40.876 |
| FEBRA2014010 | 0 | 0 | 0 | 0 | 0 | 0 | 40.243 |
| FEBRA2014122 | 0 | 0 | 0 | 0 | 0 | 0 | 66.741 |
| FEBRA2014198 | 0 | 0 | 0 | 0 | 0 | 0 | 100 |
| FEBRA2014213 | 0 | 0 | 0 | 0 | 0 | 0 | 9.817 |
| FEBRA2014297 | 0 | 0 | 0 | 0 | 0 | 0 | 5.328 |
| FEBRA2014417 | 0 | 0 | 0 | 0 | 0 | 0 | 4.038 |
| FEBRA2014443 | 0 | 0 | 0 | 0 | 0 | 0 | 100 |
| FEBRA2014578 | 0 | 0 | 0 | 0 | 0 | 0 | 54.561 |
| FEBRA2014939 | 0 | 0 | 0 | 0 | 0 | 15.432 | 20.824 |
| FEBRA2015076 | 0 | 0 | 0 | 0 | 0 | 0 | 100 |
| FEBRA2015085 | 0 | 0 | 0 | 0 | 0 | 25.351 | 7.602 |
| FEBRA2015175 | 0 | 0 | 0 | 0 | 0 | 0 | 100 |
| FEBRA2015292 | 0 | 0 | 0 | 0 | 0 | 0 | 100 |
| FEBRA2015588 | 0 | 0 | 0 | 0 | 0 | 0 | 100 |
| FEBRA2016112 | 0 | 0 | 0 | 0 | 0 | 0 | 100 |
| FEBRA2016398 | 0 | 0 | 0 | 0 | 0 | 0 | 43.076 |
| FEBRA2016572 | 0 | 0 | 0 | 0 | 0 | 0 | 48.982 |
| FEBRA2016583 | 0 | 0 | 0 | 0 | 0 | 0 | 70.959 |
| FEBRA2016621 | 0 | 0 | 0 | 0 | 0 | 0 | 100 |
| FEBRA2016654 | 0 | 0 | 0 | 0 | 0 | 0 | 100 |
| FEBRA2016739 | 0 | 0 | 0 | 0 | 0 | 0 | 100 |
| FEBRA2017024 | 0 | 0 | 0 | 0 | 0 | 0 | 24.052 |
| FEBRA2017098 | 0 | 0 | 0 | 0 | 0 | 42.565 | 57.435 |
| FEBRA2017138 | 0 | 0 | 0 | 0 | 0 | 0 | 100 |
| FEBRA2017154 | 0 | 0 | 0 | 0 | 0 | 0 | 100 |
| FEBRA2017200 | 0 | 0 | 0 | 0 | 0 | 0 | 100 |
| FEBRA2017223 | 0 | 0 | 0 | 0 | 0 | 0 | 100 |
| FEBRA2017333 | 0 | 0 | 0 | 0 | 0 | 0 | 100 |
| FEBRA2017502 | 0 | 0 | 0 | 0 | 0 | 0 | 100 |
| FEBRA2017533 | 0 | 0 | 0 | 0 | 0 | 0 | 67.995 |
| FEBRA2017680 | 0 | 0 | 0 | 0 | 0 | 0 | 23.704 |
| FEBRA2017736 | 0 | 0 | 0 | 0 | 0 | 0 | 100 |
| FEBRA2017780 | 0 | 0 | 0 | 0 | 0 | 59.713 | 40.287 |
| FEBRA2017811 | 0 | 0 | 0 | 0 | 0 | 0 | 100 |
| FEBRA2017850 | 0 | 0 | 0 | 0 | 0 | 33.093 | 44.654 |
| FEBRA2018051 | 0 | 0 | 0 | 0 | 0 | 0 | 100 |
| FEBRA2018203 | 0 | 0 | 0 | 0 | 0 | 28.033 | 37.827 |
| FEBRA2018407 | 0 | 0 | 0 | 0 | 0 | 0 | 40.386 |
| FEBRA2018433 | 0 | 0 | 0 | 0 | 0 | 0 | 77.838 |
| FEBRA2018476 | 0 | 0 | 0 | 0 | 0 | 2.019 | 8.171 |
| FEBRA2018746 | 0 | 0 | 0 | 0 | 0 | 0 | 37.287 |
| FEBRA2019172 | 0 | 0 | 0 | 0 | 0 | 0 | 100 |
| FEBRA2019242 | 0 | 0 | 0 | 0 | 0 | 0 | 20.225 |
| FEBRA2019298 | 0 | 0 | 0 | 0 | 0 | 0 | 18.103 |
| FEBRA2019389 | 0 | 0 | 0 | 0 | 0 | 0 | 40.825 |
| FEBRA2019457 | 0 | 0 | 0 | 0 | 0 | 0 | 100 |
| FEBRA2019519 | 0 | 0 | 0 | 0 | 0 | 0 | 100 |
| FEBRA2019582 | 0 | 0 | 0 | 0 | 0 | 0 | 100 |
| FEBRA2019637 | 0 | 0 | 0 | 0 | 0 | 0 | 40.753 |
| FEBRA2019663 | 0 | 0 | 0 | 0 | 0 | 0 | 21.712 |
| FEBRA2019690 | 0 | 0 | 0 | 0 | 0 | 0 | 40.243 |
| FEBRA2019711 | 0 | 0 | 0 | 0 | 0 | 0 | 5.271 |
| FEBRA2019867 | 0 | 0 | 0 | 0 | 0 | 0 | 69.211 |
| FEBRA2020406 | 0 | 0 | 0 | 0 | 0 | 42.565 | 57.435 |
| FEBRA2020566 | 0 | 0 | 0 | 0 | 0 | 0 | 10.387 |
| FEBRA2020582 | 0 | 0 | 0 | 0 | 0 | 42.565 | 57.435 |
| FEBRA2020668 | 0 | 0 | 0 | 0 | 0 | 0 | 100 |
| FEBRA2020801 | 0 | 0 | 0 | 0 | 0 | 0 | 100 |
| FEBRA2020845 | 0 | 0 | 0 | 0 | 0 | 0 | 71.370 |
| FEBRA2020886 | 0 | 0 | 0 | 0 | 0 | 0 | 60.446 |
| FEBRA2020977 | 0 | 0 | 0 | 0 | 0 | 19.470 | 13.136 |
| FEBRA2021032 | 0 | 0 | 72.292 | 0 | 0 | 0 | 13.365 |
| FEBRA2021134 | 0 | 0 | 0 | 0 | 0 | 0 | 100 |
| FEBRA2021171 | 0 | 0 | 0 | 0 | 0 | 0 | 26.519 |
| FEBRA2021339 | 0 | 0 | 0 | 0 | 0 | 0 | 79.225 |
| FEBRA2021431 | 0 | 0 | 0 | 0 | 0 | 0 | 100 |
| FEBRA2021497 | 0 | 0 | 0 | 0 | 0 | 0 | 100 |
| FEBRA2021550 | 0 | 0 | 0 | 0 | 0 | 0 | 77.838 |
| FEBRA2021571 | 0 | 0 | 0 | 0 | 0 | 0 | 100 |
| FEBRA2021636 | 0 | 0 | 0 | 0 | 0 | 0 | 50.440 |
| FEBRA2021864 | 0 | 0 | 0 | 0 | 0 | 0 | 77.838 |
| FEBRA2021892 | 0 | 0 | 0 | 0 | 0 | 0 | 100 |
| FEBRA2021908 | 0 | 0 | 0 | 0 | 0 | 0 | 100 |
| FEBRA2021966 | 0 | 0 | 0 | 0 | 0 | 7.969 | 10.753 |
| FEBRA2022013 | 0 | 0 | 0 | 0 | 0 | 0 | 23.437 |
| FEBRA2022055 | 0 | 0 | 0 | 0 | 0 | 0 | 1.326 |
| FEBRA2022148 | 0 | 0 | 0 | 0 | 0 | 0 | 100 |
| FEBRA2022204 | 0 | 0 | 0 | 0 | 0 | 0 | 100 |
| FEBRA2022255 | 0 | 0 | 0 | 0 | 0 | 0 | 100 |
| FEBRA2022322 | 0 | 0 | 0 | 0 | 0 | 0 | 100 |
| FEBRA2022504 | 0 | 0 | 0 | 0 | 0 | 0 | 4.094 |
| FEBRA2022601 | 0 | 0 | 0 | 0 | 0 | 0 | 77.838 |
| FEBRA2022696 | 0 | 0 | 0 | 0 | 0 | 0 | 52.075 |
| FEBRA2022729 | 0 | 0 | 0 | 0 | 0 | 0 | 100 |
| FEBRA2022787 | 0 | 0 | 0 | 0 | 0 | 0 | 100 |
| FEBRA2022911 | 0 | 0 | 0 | 0 | 0 | 0 | 100 |
| FEBRA2022956 | 0 | 0 | 0 | 0 | 0 | 0 | 100 |
| FEBRA2022963 | 0 | 0 | 0 | 0 | 0 | 0 | 100 |
| FEBRA2022972 | 0 | 0 | 0 | 0 | 0 | 0 | 100 |
| FEBRA2023227 | 0 | 0 | 0 | 0 | 0 | 0 | 12.767 |
| FEBRA2023285 | 0 | 0 | 0 | 0 | 0 | 0 | 100 |
| FEBRA2023351 | 0 | 0 | 0 | 0 | 0 | 0 | 100 |
| FEBRA2023377 | 0 | 0 | 0 | 0 | 0 | 0 | 19.338 |
| FEBRA2023399 | 0 | 0 | 0 | 0 | 0 | 0 | 21.881 |
| FEBRA2023498 | 0 | 0 | 0 | 0 | 0 | 0 | 100 |
| FEBRA2023550 | 0 | 0 | 0 | 0 | 0 | 0 | 40.825 |
| FEBRA2023721 | 0 | 0 | 0 | 0 | 0 | 0 | 100 |
| FEBRA2023927 | 0 | 0 | 0 | 0 | 0 | 0 | 10.153 |
| FEBRA2023989 | 0 | 0 | 0 | 0 | 0 | 0 | 100 |
| FEBRA2023990 | 0 | 0 | 0 | 0 | 0 | 0 | 100 |
| FEBRA2024019 | 0 | 0 | 0 | 0 | 0 | 2.478 | 23.402 |
| FEBRA2024136 | 0 | 0 | 0 | 0 | 0 | 0 | 28.600 |
| FEBRA2024150 | 0 | 0 | 0 | 0 | 0 | 0 | 55.970 |
| FEBRA2024343 | 0 | 0 | 0 | 0 | 0 | 0 | 100 |
| FEBRA2024570 | 0 | 0 | 0 | 0 | 0 | 0 | 25.087 |
| FEBRA2024693 | 0 | 0 | 0 | 0 | 0 | 0 | 100 |
| FEBRA2024727 | 0 | 0 | 0 | 0 | 0 | 0 | 66.741 |
| FEBRA2024744 | 0 | 0 | 0 | 0 | 0 | 0 | 100 |
| FEBRA2024797 | 0 | 0 | 0 | 0 | 0 | 0 | 100 |
| FEBRA2024987 | 0 | 0 | 0 | 0 | 0 | 0 | 24.523 |
| FEBRA2025249 | 0 | 0 | 0 | 0 | 0 | 0 | 100 |
| FEBRA2025427 | 0 | 0 | 0 | 0 | 0 | 4.579 | 24.717 |
| FEBRA2025463 | 0 | 0 | 0 | 0 | 0 | 0 | 100 |
| FEBRA2025477 | 0 | 0 | 0 | 0 | 0 | 0 | 100 |
| FEBRA2025838 | 0 | 0 | 0 | 0 | 0 | 0 | 2.048 |
| FEBRA2026474 | 0 | 0 | 0 | 0 | 0 | 0 | 100 |
| FEBRA2026582 | 0 | 0 | 0 | 0 | 0 | 0 | 57.838 |
| FEBRA2026629 | 0 | 0 | 0 | 0 | 0 | 0 | 100 |
| FEBRA2026769 | 0 | 0 | 0 | 0 | 0 | 0 | 100 |
| FEBRA2026879 | 0 | 0 | 0 | 0 | 0 | 0 | 100 |
| FEBRA2026890 | 0 | 0 | 0 | 0 | 0 | 0 | 100 |
| FEBRA2026977 | 0 | 0 | 0 | 0 | 0 | 0 | 100 |
| FEBRA2026984 | 0 | 0 | 0 | 0 | 0 | 0 | 43.714 |
| FEBRA2027082 | 0 | 0 | 0 | 0 | 0 | 0 | 100 |
| FEBRA2027297 | 0 | 0 | 0 | 0 | 0 | 0 | 100 |
| FEBRA2027352 | 0 | 0 | 0 | 0 | 0 | 0 | 100 |
| FEBRA2027364 | 0 | 0 | 0 | 0 | 0 | 0 | 100 |
| FEBRA2027609 | 0 | 0 | 0 | 0 | 0 | 0 | 100 |
| FEBRA2028158 | 0 | 0 | 0 | 0 | 0 | 0 | 68.997 |
| FEBRA2028222 | 0 | 0 | 0 | 0 | 0 | 0 | 100 |
| FEBRA2028366 | 0 | 0 | 0 | 0 | 0 | 0 | 100 |
| FEBRA2028457 | 0 | 0 | 0 | 0 | 0 | 0 | 100 |
| FEBRA2028477 | 0 | 0 | 0 | 0 | 0 | 0 | 100 |
| FEBRA2028503 | 0 | 0 | 0 | 0 | 0 | 0 | 17.429 |
| FEBRA2028516 | 0 | 0 | 0 | 0 | 0 | 0 | 2.851 |
| FELIV1000153 | 0 | 0 | 0 | 0 | 0 | 0.305 | 0 |
| HCASM1000115 | 0 | 69.583 | 0 | 0 | 0 | 1.915 | 0 |
| HCASM2000016 | 0 | 0 | 0 | 4.398 | 0 | 1.379 | 0 |
| HCASM2000363 | 0 | 0 | 0 | 4.008 | 0 | 0 | 0 |
| HCASM2005388 | 0 | 0 | 47.694 | 0 | 0 | 0 | 0 |
| HCHON1000015 | 0 | 0 | 0 | 9.186 | 0 | 2.880 | 0 |
| HCHON1000030 | 2.118 | 0 | 0 | 0 | 4.970 | 0.670 | 1.356 |
| HCHON1000166 | 0 | 0 | 0 | 0 | 0 | 0 | 7.499 |
| HCHON2000028 | 0 | 0 | 0 | 9.350 | 0 | 0 | 0 |
| HCHON2000056 | 3.311 | 0 | 0 | 1.671 | 0 | 0 | 0.707 |
| HCHON2000160 | 0 | 0 | 0 | 5.932 | 0 | 0 | 12.547 |
| HCHON2000212 | 0 | 0 | 0 | 0 | 0 | 15.878 | 0 |
| HCHON2000244 | 0 | 0 | 0 | 7.753 | 0 | 1.215 | 4.920 |
| HCHON2000295 | 0 | 0 | 0 | 0 | 12.003 | 0 | 0 |
| HCHON2000323 | 0 | 0 | 0 | 0 | 0 | 3.422 | 0 |
| HCHON2000508 | 4.360 | 50.138 | 0 | 2.201 | 0 | 0 | 0 |
| HCHON2000676 | 0 | 0 | 0 | 17.571 | 0 | 5.508 | 0 |
| HCHON2000698 | 0 | 0 | 0 | 0 | 0 | 5.295 | 0 |
| HCHON2000738 | 0 | 0 | 0 | 0 | 0 | 5.501 | 0 |
| HCHON2000743 | 0 | 0 | 0 | 0 | 0 | 11.280 | 0 |
| HCHON2000751 | 0 | 0 | 0 | 0 | 0 | 6.596 | 0 |
| HCHON2000826 | 0 | 0 | 0 | 0 | 0 | 5.289 | 1.784 |
| HCHON2000832 | 0 | 0 | 0 | 0 | 0 | 6.536 | 8.819 |
| HCHON2000942 | 0 | 0 | 0 | 0 | 0 | 0 | 28.262 |
| HCHON2001039 | 0 | 0 | 0 | 0 | 0 | 4.123 | 2.782 |
| HCHON2001050 | 16.922 | 0 | 0 | 0 | 0 | 0 | 0 |
| HCHON2001099 | 0 | 0 | 0 | 48.221 | 0 | 0 | 0 |
| HCHON2001200 | 0 | 0 | 0 | 0 | 0 | 4.794 | 0 |
| HCHON2001217 | 0 | 0 | 0 | 6.134 | 0 | 3.846 | 2.595 |
| HCHON2001407 | 0 | 0 | 0 | 0 | 0 | 4.377 | 1.969 |
| HCHON2001434 | 0 | 0 | 0 | 0 | 0 | 5.644 | 0 |
| HCHON2001665 | 0 | 0 | 0 | 0 | 0 | 2.851 | 0 |
| HCHON2002496 | 0 | 0 | 0 | 45.775 | 0 | 0 | 0 |
| HCHON2004002 | 0 | 0 | 0 | 0 | 0 | 0 | 1.957 |
| HCHON2005166 | 0 | 0 | 0 | 0 | 0 | 22.599 | 0 |
| HCHON2006770 | 0 | 0 | 0 | 16.633 | 0 | 0 | 0 |
| HCHON2010543 | 0 | 0 | 0 | 0 | 0 | 16.573 | 0 |
| HEART1000149 | 0 | 0 | 0 | 5.534 | 0 | 1.735 | 0 |
| HEART1000173 | 0 | 0 | 0 | 0 | 0 | 4.849 | 0 |
| HEART2006521 | 0 | 0 | 0 | 0 | 0 | 8.837 | 0 |
| HEART2007231 | 0 | 0 | 0 | 0 | 0 | 8.949 | 0 |
| HHDPC1000083 | 0 | 0 | 0 | 0 | 0 | 0 | 2.440 |
| HHDPC1000114 | 0 | 0 | 0 | 0 | 0 | 0 | 1.811 |
| HHDPC1000118 | 0 | 0 | 0 | 0 | 0 | 15.487 | 5.225 |
| HHDPC1000163 | 0 | 0 | 0 | 14.822 | 0 | 4.646 | 0 |
| HHDPC2000055 | 0 | 0 | 0 | 0 | 0 | 6.756 | 4.558 |
| HHDPC2000104 | 0 | 0 | 0 | 10.903 | 0 | 0 | 0 |
| HHDPC2000149 | 0 | 0 | 0 | 0 | 0 | 8.255 | 0 |
| HHDPC2000455 | 0 | 0 | 0 | 6.477 | 0 | 0 | 0 |
| HHDPC2003439 | 0 | 0 | 0 | 7.494 | 0 | 2.819 | 1.268 |
| HHDPC2003983 | 0 | 0 | 0 | 0 | 0 | 2.572 | 0.694 |
| HHDPC2006862 | 0 | 0 | 0 | 0 | 0 | 13.827 | 0 |
| HHDPC2007267 | 0 | 0 | 0 | 0 | 0 | 0.902 | 0 |
| HHDPC2009528 | 0 | 0 | 0 | 0 | 0 | 12.007 | 0 |
| HLUNG1000055 | 0 | 0 | 0 | 3.190 | 0 | 1.000 | 1.349 |
| HLUNG1000091 | 0 | 0 | 0 | 0 | 0 | 27.339 | 0 |
| HLUNG2000063 | 0 | 0 | 0 | 0 | 0 | 0 | 6.148 |
| HLUNG2000232 | 0 | 0 | 0 | 0 | 0 | 23.283 | 0 |
| HLUNG2000314 | 0 | 0 | 0 | 0 | 67.223 | 0 | 0 |
| HLUNG2000412 | 0 | 0 | 0 | 0 | 0 | 0 | 6.675 |
| HLUNG2000501 | 0 | 0 | 0 | 0 | 0 | 1.585 | 0 |
| HLUNG2000884 | 0 | 0 | 0 | 0 | 0 | 11.863 | 5.336 |
| HLUNG2000950 | 0 | 0 | 0 | 0 | 0 | 22.840 | 0 |
| HLUNG2001439 | 0 | 0 | 0 | 5.197 | 0 | 1.629 | 0 |
| HLUNG2001518 | 0 | 0 | 0 | 0 | 0 | 0 | 5.452 |
| HLUNG2002059 | 0 | 0 | 0 | 48.430 | 0 | 0 | 0 |
| HLUNG2002334 | 0 | 0 | 0 | 0 | 0 | 16.982 | 0 |
| HLUNG2004159 | 0 | 0 | 0 | 0 | 0 | 6.699 | 4.520 |
| HLUNG2004774 | 0 | 0 | 0 | 0 | 0 | 0 | 40.465 |
| HLUNG2005230 | 26.687 | 0 | 0 | 0 | 0 | 0 | 0 |
| HLUNG2006812 | 0 | 0 | 0 | 0 | 0 | 5.463 | 0 |
| HLUNG2008885 | 0 | 0 | 0 | 0 | 0 | 0 | 26.910 |
| HLUNG2009225 | 0 | 0 | 0 | 0 | 0 | 5.572 | 3.760 |
| HLUNG2017262 | 0 | 0 | 0 | 9.346 | 0 | 5.859 | 3.953 |
| HSYRA1000062 | 0 | 0 | 0 | 10.097 | 0 | 1.583 | 2.136 |
| HSYRA1000119 | 0 | 0 | 0 | 2.710 | 0 | 0 | 0 |
| HSYRA1000152 | 0 | 0 | 0 | 7.029 | 0 | 6.611 | 0 |
| HSYRA2000159 | 0 | 0 | 1.626 | 0 | 0 | 1.782 | 1.202 |
| HSYRA2000190 | 0 | 0 | 0 | 13.997 | 0 | 0 | 0 |
| HSYRA2000221 | 0 | 0 | 0 | 0 | 0 | 17.488 | 0 |
| HSYRA2000232 | 0 | 0 | 0 | 0 | 0 | 19.125 | 0 |
| HSYRA2000248 | 0 | 0 | 0 | 0 | 0 | 3.767 | 8.471 |
| HSYRA2000332 | 0 | 0 | 0 | 0 | 0 | 0 | 1.944 |
| HSYRA2000640 | 0 | 0 | 0 | 5.021 | 0 | 0 | 6.371 |
| HSYRA2000743 | 0 | 0 | 0 | 9.121 | 0 | 0 | 0 |
| HSYRA2000787 | 0 | 0 | 0 | 0 | 0 | 0 | 15.507 |
| HSYRA2001103 | 0 | 0 | 0 | 14.995 | 0 | 0 | 0 |
| HSYRA2001138 | 0 | 0 | 0 | 3.005 | 0 | 0 | 0 |
| HSYRA2001225 | 0 | 0 | 0 | 0 | 0 | 0 | 3.686 |
| HSYRA2001396 | 0 | 0 | 0 | 0 | 0 | 0 | 3.787 |
| HSYRA2001476 | 0 | 0 | 0 | 0 | 0 | 0.892 | 0 |
| HSYRA2001567 | 0 | 0 | 0 | 0 | 0 | 4.140 | 0 |
| HSYRA2001574 | 0 | 0 | 0 | 0 | 0 | 0 | 3.751 |
| HSYRA2001580 | 0 | 0 | 0 | 32.300 | 0 | 0 | 13.663 |
| HSYRA2001595 | 0 | 0 | 0 | 0 | 0 | 2.105 | 1.420 |
| HSYRA2001615 | 0 | 0 | 0 | 12.222 | 0 | 0 | 0 |
| HSYRA2001621 | 0 | 0 | 0 | 0 | 0 | 0.339 | 0.457 |
| HSYRA2001631 | 0 | 0 | 0 | 0 | 0 | 21.545 | 0 |
| IMR321000193 | 0 | 0 | 0 | 25.866 | 0 | 8.109 | 0 |
| IMR321000210 | 0 | 0 | 0 | 0 | 0 | 3.745 | 7.580 |
| IMR321000219 | 0 | 0 | 0 | 1.654 | 0 | 1.037 | 0 |
| IMR321000242 | 0 | 0 | 0 | 0 | 0 | 10.389 | 14.019 |
| IMR321000253 | 0 | 0 | 0 | 0 | 0 | 3.763 | 0 |
| IMR322000121 | 0 | 0 | 0 | 6.570 | 0 | 0 | 0 |
| IMR322000127 | 0 | 0 | 0 | 0 | 0 | 8.585 | 11.585 |
| IMR322000144 | 0 | 0 | 0 | 0 | 44.695 | 0 | 0 |
| IMR322000223 | 7.949 | 0 | 0 | 0 | 0 | 0 | 3.395 |
| IMR322000243 | 0 | 0 | 0 | 5.056 | 0 | 0 | 0 |
| IMR322000316 | 0 | 0 | 0 | 0 | 0 | 9.199 | 16.551 |
| IMR322000555 | 0 | 0 | 0 | 0 | 0 | 11.022 | 0 |
| IMR322000730 | 1.600 | 9.201 | 0 | 0 | 0 | 0 | 0 |
| IMR322000819 | 49.762 | 0 | 0 | 0 | 0 | 0 | 0 |
| IMR322000917 | 0 | 0 | 0 | 0 | 0 | 43.645 | 0 |
| IMR322000918 | 0 | 0 | 0 | 34.549 | 0 | 0 | 0 |
| IMR322000935 | 0 | 0 | 0 | 8.823 | 0 | 2.766 | 0 |
| IMR322000973 | 0 | 0 | 0 | 4.752 | 0 | 1.490 | 0 |
| IMR322001218 | 13.070 | 0 | 0 | 0 | 0 | 2.068 | 0 |
| IMR322001332 | 0 | 0 | 0 | 0 | 0 | 4.919 | 0 |
| IMR322001380 | 0 | 0 | 0 | 0 | 0 | 7.324 | 9.883 |
| IMR322001435 | 0 | 0 | 0 | 0 | 0 | 3.471 | 0 |
| IMR322001541 | 0 | 0 | 0 | 0 | 0 | 6.251 | 0 |
| IMR322001584 | 0 | 0 | 16.832 | 0 | 0 | 0 | 3.112 |
| IMR322001600 | 0 | 10.810 | 0 | 6.643 | 0 | 0.297 | 0.401 |
| IMR322001670 | 0 | 0 | 0 | 0 | 0 | 16.857 | 0 |
| IMR322012529 | 0 | 0 | 0 | 0 | 0 | 8.700 | 0 |
| IMR322019070 | 0 | 0 | 0 | 0 | 0 | 0 | 13.339 |
| KIDNE2000085 | 0 | 0 | 0 | 0 | 28.178 | 0 | 0 |
| KIDNE2000252 | 0 | 0 | 0 | 0 | 0 | 1.579 | 0 |
| KIDNE2000403 | 0 | 0 | 0 | 4.869 | 0 | 1.526 | 0 |
| KIDNE2000574 | 0 | 0 | 0 | 0 | 0 | 0 | 3.732 |
| KIDNE2000665 | 0 | 0 | 0 | 0 | 0 | 3.576 | 0 |
| KIDNE2000947 | 0 | 0 | 0 | 14.895 | 0 | 9.338 | 0 |
| KIDNE2001162 | 0 | 0 | 0 | 0 | 0 | 0 | 1.164 |
| KIDNE2001467 | 0 | 0 | 0 | 3.072 | 0 | 3.371 | 1.949 |
| KIDNE2001713 | 0 | 0 | 0 | 0 | 0 | 28.625 | 0 |
| KIDNE2002265 | 0 | 0 | 0 | 0 | 0 | 10.080 | 4.534 |
| KIDNE2002438 | 0 | 0 | 0 | 0 | 0 | 34.637 | 0 |
| KIDNE2002872 | 0 | 38.385 | 46.259 | 0 | 0 | 0 | 0 |
| KIDNE2002883 | 0 | 0 | 0 | 0 | 0 | 8.380 | 0 |
| KIDNE2003373 | 0 | 0 | 0 | 0 | 0 | 28.625 | 0 |
| KIDNE2003941 | 0 | 0 | 0 | 2.972 | 0 | 0 | 1.257 |
| KIDNE2004305 | 0 | 0 | 0 | 0 | 0 | 34.637 | 0 |
| KIDNE2004475 | 0 | 0 | 0 | 0 | 0 | 4.755 | 19.250 |
| KIDNE2004626 | 0 | 0 | 0 | 0 | 0 | 0 | 32.129 |
| KIDNE2004846 | 77.005 | 0 | 0 | 0 | 0 | 0 | 0 |
| KIDNE2005336 | 0 | 0 | 0 | 0 | 0 | 0 | 41.693 |
| KIDNE2005676 | 0 | 0 | 0 | 8.291 | 0 | 10.396 | 0 |
| KIDNE2006014 | 0 | 0 | 0 | 0 | 0 | 10.971 | 7.402 |
| KIDNE2006248 | 3.630 | 0 | 4.192 | 1.832 | 0 | 0 | 0 |
| KIDNE2006465 | 0 | 0 | 0 | 0 | 0 | 5.716 | 15.427 |
| KIDNE2006811 | 37.991 | 0 | 0 | 0 | 0 | 0 | 8.112 |
| KIDNE2006880 | 0 | 0 | 43.291 | 0 | 0 | 0 | 0 |
| KIDNE2007352 | 0 | 0 | 0 | 0 | 0 | 2.304 | 2.073 |
| KIDNE2008403 | 0 | 0 | 0 | 0 | 0 | 0 | 3.390 |
| KIDNE2008404 | 0 | 0 | 62.526 | 0 | 0 | 0 | 0 |
| KIDNE2009367 | 0 | 0 | 0 | 4.442 | 0 | 0 | 0 |
| KIDNE2010265 | 0 | 0 | 63.715 | 0 | 0 | 0 | 5.890 |
| KIDNE2013413 | 0 | 0 | 0 | 0 | 0 | 34.637 | 0 |
| KIDNE2013845 | 0 | 0 | 0 | 0 | 0 | 2.065 | 0 |
| KIDNE2014290 | 0 | 0 | 0 | 0 | 0 | 0 | 1.748 |
| KIDNE2015445 | 0 | 0 | 0 | 20.247 | 0 | 0 | 0 |
| KIDNE2018352 | 0 | 0 | 0 | 0 | 0 | 6.588 | 0 |
| KIDNE2018493 | 0 | 0 | 0 | 0 | 0 | 0 | 17.765 |
| LIVER1000132 | 5.842 | 0 | 0 | 0 | 0 | 0.924 | 2.495 |
| LIVER1000230 | 0 | 0 | 0 | 0 | 0 | 0 | 14.457 |
| LIVER2000416 | 0 | 0 | 0 | 0 | 0 | 4.180 | 0 |
| LIVER2001539 | 0 | 0 | 0 | 0 | 0 | 9.200 | 0 |
| LIVER2002842 | 0 | 0 | 0 | 0 | 0 | 3.527 | 9.518 |
| LIVER2006006 | 0 | 47.741 | 0 | 8.382 | 0 | 1.314 | 0 |
| MAMGL1000032 | 0 | 0 | 0 | 0 | 0 | 0.298 | 0.402 |
| MAMGL1000035 | 0 | 0 | 0 | 2.373 | 0 | 0.372 | 0 |
| MESAN1000050 | 0 | 0 | 0 | 28.382 | 0 | 0 | 4.002 |
| MESAN1000101 | 0 | 0 | 0 | 0 | 0 | 0 | 14.389 |
| MESAN2000067 | 0 | 0 | 0 | 0 | 0 | 33.254 | 0 |
| MESAN2000092 | 0 | 0 | 0 | 0 | 0 | 16.348 | 0 |
| MESAN2000149 | 0 | 0 | 0 | 14.477 | 0 | 4.538 | 0 |
| MESAN2000267 | 0 | 0 | 0 | 0 | 0 | 1.513 | 1.021 |
| MESAN2000572 | 0 | 0 | 0 | 0 | 0 | 1.889 | 0 |
| MESAN2001450 | 0 | 0 | 0 | 0 | 0 | 5.328 | 0 |
| MESAN2002086 | 0 | 0 | 0 | 7.360 | 0 | 0 | 0 |
| MESAN2002186 | 0 | 0 | 59.118 | 0 | 0 | 0 | 0 |
| MESAN2002519 | 0 | 0 | 0 | 0 | 0 | 0 | 11.561 |
| MESAN2002724 | 0 | 0 | 12.435 | 0 | 0 | 0 | 0 |
| MESAN2002790 | 0 | 0 | 0 | 28.827 | 0 | 0 | 0 |
| MESAN2002844 | 0 | 0 | 0 | 0 | 0 | 0 | 18.063 |
| MESAN2003035 | 0 | 0 | 0 | 0 | 0 | 13.240 | 0 |
| MESAN2003622 | 0 | 0 | 0 | 3.542 | 0 | 0 | 1.498 |
| MESAN2004288 | 0 | 0 | 0 | 30.624 | 0 | 9.600 | 0 |
| MESAN2004575 | 0 | 0 | 0 | 76.069 | 0 | 0 | 0 |
| MESAN2005689 | 0 | 0 | 0 | 0 | 0 | 5.732 | 0 |
| MESAN2005766 | 0 | 0 | 0 | 0 | 0 | 3.644 | 0 |
| MESAN2006401 | 0 | 0 | 0 | 0 | 0 | 3.342 | 0 |
| MESAN2006563 | 0 | 0 | 0 | 0 | 0 | 3.827 | 0 |
| MESAN2006971 | 0 | 0 | 0 | 35.411 | 0 | 0 | 0 |
| MESAN2007755 | 0 | 0 | 0 | 0 | 0 | 16.901 | 0 |
| MESAN2008926 | 0 | 0 | 0 | 0 | 0 | 5.898 | 0 |
| MESAN2008936 | 0 | 0 | 0 | 0 | 0 | 0 | 9.917 |
| MESAN2009081 | 0 | 0 | 0 | 27.740 | 0 | 8.696 | 0 |
| MESAN2011627 | 0 | 0 | 50.747 | 0 | 0 | 0 | 0 |
| MESAN2011984 | 0 | 0 | 0 | 0 | 0 | 6.304 | 0 |
| MESAN2012054 | 0 | 0 | 0 | 0 | 0 | 4.713 | 0 |
| MESAN2013284 | 0 | 0 | 0 | 0 | 0 | 11.345 | 0 |
| NB9N41000011 | 0 | 0 | 0 | 12.543 | 0 | 0 | 0 |
| NB9N42000104 | 0 | 0 | 0 | 9.770 | 0 | 0 | 0 |
| NB9N42000281 | 0 | 0 | 0 | 0 | 0 | 3.532 | 0 |
| NHNPC2000187 | 5.283 | 0 | 0 | 0 | 12.400 | 0 | 0 |
| NHNPC2000206 | 0 | 0 | 0 | 4.047 | 0 | 1.269 | 1.712 |
| NHNPC2000212 | 0 | 0 | 0 | 0 | 0 | 6.438 | 0 |
| NHNPC2001223 | 0 | 0 | 0 | 0 | 0 | 6.894 | 0 |
| NHNPC2001931 | 0 | 0 | 0 | 2.813 | 0 | 0 | 0 |
| NT2NE1000018 | 0 | 0 | 0 | 0 | 0 | 4.594 | 6.199 |
| NT2NE1000024 | 0 | 0 | 0 | 0 | 0 | 0 | 28.710 |
| NT2NE1000073 | 0 | 0 | 0 | 0 | 0 | 0 | 19.850 |
| NT2NE1000163 | 0 | 0 | 5.462 | 0 | 0 | 0 | 0 |
| NT2NE2000056 | 0 | 0 | 0 | 0 | 0 | 2.177 | 3.917 |
| NT2NE2000260 | 1.410 | 0 | 0 | 0 | 0 | 0.223 | 0 |
| NT2NE2000327 | 0 | 0 | 0 | 0 | 0 | 0 | 12.542 |
| NT2NE2000353 | 0 | 0 | 0 | 0 | 0 | 33.765 | 0 |
| NT2NE2000383 | 0 | 0 | 0 | 0 | 0 | 0 | 3.788 |
| NT2NE2000386 | 0 | 0 | 0 | 0 | 0 | 28.547 | 0 |
| NT2NE2000392 | 0 | 0 | 0 | 0 | 0 | 24.740 | 24.481 |
| NT2NE2000470 | 0 | 0 | 0 | 0 | 0 | 10.082 | 0 |
| NT2NE2000575 | 0 | 0 | 0 | 0 | 0 | 0.888 | 0.599 |
| NT2NE2000707 | 0 | 0 | 0 | 0 | 0 | 3.091 | 1.668 |
| NT2NE2000763 | 0 | 0 | 0 | 0 | 32.885 | 0 | 2.992 |
| NT2NE2000809 | 0 | 0 | 0 | 19.976 | 0 | 0 | 8.450 |
| NT2NE2000909 | 0 | 0 | 0 | 0 | 0 | 12.095 | 8.160 |
| NT2NE2001000 | 0 | 0 | 0 | 5.072 | 0 | 0 | 0 |
| NT2NE2001048 | 0 | 0 | 0 | 0 | 0 | 2.765 | 0 |
| NT2NE2001176 | 0 | 0 | 0 | 0 | 0 | 6.330 | 2.847 |
| NT2NE2001337 | 0 | 0 | 0 | 0 | 0 | 16.790 | 0 |
| NT2NE2001435 | 0 | 0 | 0 | 0 | 0 | 7.856 | 5.300 |
| NT2NE2001442 | 0 | 0 | 0 | 0 | 0 | 9.232 | 0 |
| NT2NE2001530 | 0 | 0 | 0 | 0 | 0 | 12.926 | 0 |
| NT2NE2001545 | 0 | 0 | 0 | 10.559 | 0 | 0 | 0 |
| NT2NE2001617 | 0 | 0 | 0 | 0 | 0 | 8.382 | 0 |
| NT2NE2001793 | 0 | 0 | 0 | 0 | 0 | 1.916 | 0 |
| NT2NE2003185 | 0 | 0 | 0 | 0 | 0 | 14.006 | 0 |
| NT2NE2003280 | 0 | 0 | 0 | 0 | 0 | 13.445 | 0 |
| NT2NE2003393 | 0 | 86.291 | 0 | 0 | 0 | 0 | 0 |
| NT2NE2003485 | 0 | 0 | 62.774 | 0 | 0 | 0 | 0 |
| NT2NE2003569 | 0 | 0 | 0 | 10.228 | 0 | 6.412 | 0 |
| NT2NE2004255 | 0 | 0 | 0 | 35.102 | 0 | 0 | 14.848 |
| NT2NE2004519 | 0 | 0 | 0 | 0 | 0 | 6.621 | 0 |
| NT2NE2005371 | 0 | 0 | 0 | 0 | 0 | 0 | 13.745 |
| NT2NE2005395 | 0 | 0 | 0 | 0 | 0 | 50.484 | 0 |
| NT2NE2005688 | 0 | 0 | 0 | 0 | 0 | 4.139 | 0 |
| NT2NE2005876 | 0 | 0 | 0 | 0 | 0 | 5.450 | 14.709 |
| NT2NE2006087 | 0 | 0 | 0 | 0 | 0 | 0 | 50.606 |
| NT2NE2006103 | 0 | 0 | 0 | 0 | 0 | 15.996 | 0 |
| NT2NE2006458 | 0 | 0 | 0 | 0 | 0 | 2.611 | 0 |
| NT2NE2006909 | 0 | 0 | 0 | 0 | 0 | 0.527 | 0.711 |
| NT2NE2006958 | 0 | 0 | 0 | 0 | 0 | 0 | 40.753 |
| NT2NE2008213 | 0 | 0 | 0 | 0 | 0 | 0 | 20.745 |
| NT2NE2009295 | 0 | 0 | 0 | 11.757 | 0 | 0 | 4.973 |
| NT2NE2010056 | 0 | 0 | 0 | 0 | 0 | 0 | 19.976 |
| NT2NE2010842 | 0 | 0 | 0 | 0 | 0 | 3.484 | 2.350 |
| NT2NE2011033 | 0 | 0 | 0 | 0 | 0 | 33.765 | 0 |
| NT2NE2014104 | 0 | 0 | 0 | 9.892 | 0 | 0 | 4.184 |
| NT2NE2014113 | 0 | 0 | 0 | 0 | 0 | 33.527 | 0 |
| NT2NE2019091 | 0 | 0 | 0 | 0 | 0 | 0 | 4.719 |
| NT2RI1000048 | 0 | 57.002 | 0 | 0 | 0 | 1.569 | 6.350 |
| NT2RI1000127 | 0 | 0 | 0 | 0 | 0 | 10.059 | 9.049 |
| NT2RI2000064 | 12.568 | 0 | 0 | 0 | 0 | 1.989 | 0 |
| NT2RI2000116 | 0 | 0 | 0 | 0 | 0 | 21.488 | 0 |
| NT2RI2000133 | 29.915 | 0 | 0 | 0 | 0 | 0 | 0 |
| NT2RI2000255 | 0 | 0 | 0 | 7.054 | 0 | 0 | 0 |
| NT2RI2000282 | 0 | 0 | 0 | 3.731 | 0 | 0 | 0 |
| NT2RI2000294 | 0 | 0 | 0 | 0 | 0 | 3.497 | 9.436 |
| NT2RI2000344 | 0 | 0 | 0 | 0 | 0 | 0 | 4.037 |
| NT2RI2000348 | 0 | 0 | 0 | 0 | 0 | 16.371 | 0 |
| NT2RI2000412 | 0 | 0 | 0 | 0 | 0 | 14.239 | 0 |
| NT2RI2000578 | 0 | 0 | 4.509 | 3.941 | 0 | 0 | 1.667 |
| NT2RI2000685 | 0 | 0 | 0 | 0 | 0 | 3.661 | 2.470 |
| NT2RI2000727 | 0 | 0 | 0 | 0 | 0 | 1.434 | 3.225 |
| NT2RI2000829 | 0 | 0 | 0 | 0 | 0 | 7.056 | 0 |
| NT2RI2001054 | 6.946 | 0 | 0 | 0 | 0 | 0 | 1.483 |
| NT2RI2001091 | 41.046 | 0 | 0 | 0 | 0 | 6.495 | 0 |
| NT2RI2001342 | 0 | 0 | 0 | 0 | 0 | 2.916 | 0 |
| NT2RI2001385 | 0 | 0 | 0 | 0 | 0 | 7.279 | 0 |
| NT2RI2001422 | 0 | 0 | 50.129 | 0 | 0 | 0 | 0 |
| NT2RI2001450 | 0 | 0 | 0 | 0 | 0 | 6.547 | 17.668 |
| NT2RI2001540 | 0.665 | 0 | 1.535 | 0.335 | 0 | 0.631 | 1.277 |
| NT2RI2001621 | 0 | 0 | 0 | 18.040 | 0 | 5.655 | 0 |
| NT2RI2001657 | 6.247 | 0 | 7.215 | 0 | 0 | 0.988 | 0 |
| NT2RI2001731 | 0 | 0 | 0 | 0 | 0 | 3.499 | 0 |
| NT2RI2001859 | 0 | 0 | 0 | 5.719 | 0 | 1.793 | 0 |
| NT2RI2001952 | 0 | 0 | 0 | 0 | 0 | 38.701 | 0 |
| NT2RI2002091 | 0.470 | 0 | 0.543 | 4.110 | 2.205 | 0.025 | 0.033 |
| NT2RI2002117 | 0 | 0 | 0 | 0 | 0 | 0 | 11.744 |
| NT2RI2002152 | 0 | 0 | 0 | 7.411 | 0 | 0 | 0 |
| NT2RI2002316 | 0 | 0 | 10.811 | 9.450 | 0 | 1.481 | 1.999 |
| NT2RI2002391 | 0 | 0 | 0 | 0 | 0 | 4.495 | 4.043 |
| NT2RI2002540 | 0 | 0 | 0 | 0 | 0 | 3.194 | 17.238 |
| NT2RI2002554 | 0 | 0 | 0 | 10.686 | 0 | 6.700 | 9.040 |
| NT2RI2002654 | 0 | 0 | 0 | 0 | 0 | 1.085 | 0 |
| NT2RI2002699 | 0 | 0 | 0 | 0 | 0 | 21.123 | 0 |
| NT2RI2002819 | 0 | 0 | 0 | 0 | 0 | 0 | 9.281 |
| NT2RI2002847 | 0 | 0 | 0 | 0 | 0 | 6.957 | 9.388 |
| NT2RI2002958 | 0 | 0 | 0 | 0 | 0 | 0 | 14.400 |
| NT2RI2003019 | 0 | 0 | 0 | 30.822 | 0 | 0 | 0 |
| NT2RI2003051 | 0 | 0 | 0 | 0 | 0 | 1.891 | 0 |
| NT2RI2003301 | 0 | 0 | 0 | 0 | 0 | 10.486 | 14.149 |
| NT2RI2003304 | 0 | 0 | 0 | 0 | 0 | 0.485 | 0.654 |
| NT2RI2003317 | 0 | 0 | 0 | 9.290 | 0 | 2.912 | 3.929 |
| NT2RI2003361 | 0 | 0 | 0 | 0 | 0 | 0 | 12.312 |
| NT2RI2003383 | 0 | 0 | 0 | 0 | 0 | 67.032 | 0 |
| NT2RI2003407 | 0 | 0 | 0 | 0 | 0 | 2.236 | 0 |
| NT2RI2003420 | 0 | 0 | 0 | 9.075 | 0 | 5.690 | 3.839 |
| NT2RI2003556 | 4.442 | 0 | 0 | 0 | 0 | 0 | 0.948 |
| NT2RI2003695 | 0 | 0 | 0 | 0 | 0 | 7.274 | 0 |
| NT2RI2003738 | 0 | 0 | 0 | 3.093 | 19.175 | 0.646 | 1.744 |
| NT2RI2003751 | 0 | 0 | 0 | 0 | 0 | 2.559 | 3.453 |
| NT2RI2003921 | 0 | 0 | 0 | 2.795 | 12.995 | 0.876 | 1.182 |
| NT2RI2003973 | 0 | 0 | 0 | 0 | 0 | 23.190 | 0 |
| NT2RI2003993 | 0 | 0 | 0 | 0 | 0 | 0 | 9.464 |
| NT2RI2004209 | 0 | 0 | 0 | 0 | 0 | 0 | 18.302 |
| NT2RI2004230 | 0 | 0 | 0 | 0 | 0 | 8.479 | 2.860 |
| NT2RI2004398 | 0 | 0 | 0 | 0 | 0 | 0 | 12.419 |
| NT2RI2004410 | 0 | 0 | 0 | 0 | 0 | 9.215 | 0 |
| NT2RI2004884 | 0 | 0 | 0 | 0 | 0 | 0 | 11.556 |
| NT2RI2005116 | 0 | 0 | 0 | 0 | 0 | 2.362 | 0 |
| NT2RI2005150 | 0 | 0 | 0 | 0 | 0 | 0 | 1.718 |
| NT2RI2005335 | 0 | 0 | 0 | 6.844 | 0 | 2.145 | 0 |
| NT2RI2005353 | 0 | 0 | 0 | 6.789 | 0 | 0 | 2.872 |
| NT2RI2005368 | 0 | 0 | 0 | 0 | 0 | 3.733 | 0 |
| NT2RI2005405 | 0 | 0 | 0 | 6.617 | 0 | 0.519 | 1.400 |
| NT2RI2005564 | 0 | 0 | 0 | 0 | 0 | 0 | 22.207 |
| NT2RI2005579 | 0 | 0 | 0 | 0 | 0 | 17.338 | 0 |
| NT2RI2005713 | 0 | 0 | 0 | 8.715 | 0 | 5.464 | 3.687 |
| NT2RI2005723 | 0 | 0 | 0 | 2.957 | 0 | 1.854 | 2.502 |
| NT2RI2005811 | 17.886 | 0 | 0 | 0 | 0 | 5.661 | 0 |
| NT2RI2005816 | 0 | 0 | 0 | 0 | 0 | 11.809 | 0 |
| NT2RI2005818 | 0 | 0 | 0 | 0 | 0 | 14.116 | 19.048 |
| NT2RI2006072 | 0 | 0 | 0 | 4.509 | 0 | 0 | 0 |
| NT2RI2006183 | 0 | 0 | 0 | 0 | 0 | 0 | 47.686 |
| NT2RI2006412 | 0 | 0 | 0 | 0 | 0 | 0 | 2.043 |
| NT2RI2006506 | 0 | 0 | 0 | 0 | 0 | 24.887 | 0 |
| NT2RI2006647 | 0 | 0 | 0 | 27.023 | 0 | 8.471 | 0 |
| NT2RI2006825 | 0 | 0 | 0 | 0 | 0 | 15.195 | 6.834 |
| NT2RI2006856 | 0 | 0 | 0 | 0 | 0 | 0 | 2.335 |
| NT2RI2006943 | 0 | 0 | 0 | 0 | 0 | 4.494 | 9.096 |
| NT2RI2007054 | 0 | 0 | 0 | 0 | 0 | 2.398 | 0 |
| NT2RI2007133 | 0 | 0 | 0 | 0 | 0 | 0 | 0.978 |
| NT2RI2007277 | 0 | 0 | 0 | 0 | 0 | 10.867 | 9.775 |
| NT2RI2007377 | 0 | 0 | 0 | 0 | 0 | 12.982 | 11.678 |
| NT2RI2007384 | 0 | 0 | 0 | 5.104 | 0 | 0 | 0 |
| NT2RI2007468 | 0 | 0 | 0 | 0 | 0 | 7.719 | 0 |
| NT2RI2007572 | 0 | 0 | 0 | 8.807 | 0 | 5.521 | 1.863 |
| NT2RI2007589 | 0 | 0 | 0 | 0 | 0 | 0 | 15.973 |
| NT2RI2007629 | 0 | 0 | 0 | 11.880 | 0 | 3.724 | 5.025 |
| NT2RI2007723 | 0 | 0 | 0 | 0 | 0 | 0 | 8.819 |
| NT2RI2007884 | 0 | 0 | 0 | 0 | 0 | 4.690 | 0 |
| NT2RI2007987 | 0 | 0 | 0 | 0 | 0 | 0 | 8.977 |
| NT2RI2008045 | 0 | 0 | 10.470 | 0 | 0 | 2.869 | 3.871 |
| NT2RI2008171 | 0 | 0 | 0 | 0 | 0 | 8.203 | 0 |
| NT2RI2008221 | 0 | 74.427 | 0 | 0 | 0 | 0 | 0 |
| NT2RI2008336 | 0 | 0 | 0 | 0 | 0 | 2.960 | 1.997 |
| NT2RI2008396 | 0 | 0 | 0 | 2.598 | 0 | 0.814 | 3.296 |
| NT2RI2008526 | 0 | 0 | 0 | 4.518 | 0 | 0 | 0 |
| NT2RI2008566 | 0 | 0 | 0 | 0 | 0 | 0 | 23.447 |
| NT2RI2008724 | 0 | 0 | 0 | 0 | 0 | 3.393 | 0 |
| NT2RI2008749 | 0 | 0 | 0 | 0 | 0 | 1.045 | 0 |
| NT2RI2008791 | 0 | 0 | 0 | 0 | 0 | 2.155 | 0 |
| NT2RI2008808 | 0 | 0 | 0 | 0 | 0 | 33.500 | 11.301 |
| NT2RI2008942 | 0 | 0 | 0 | 0 | 0 | 1.937 | 5.228 |
| NT2RI2009037 | 0 | 0 | 0 | 0 | 0 | 1.557 | 8.406 |
| NT2RI2009065 | 0 | 0 | 0 | 0 | 0 | 2.588 | 0 |
| NT2RI2009173 | 0 | 0 | 0 | 0 | 0 | 2.061 | 5.561 |
| NT2RI2009194 | 0 | 0 | 0 | 17.375 | 0 | 1.816 | 9.799 |
| NT2RI2009233 | 0 | 0 | 0 | 0 | 0 | 4.167 | 0 |
| NT2RI2009239 | 0 | 0 | 0 | 0 | 0 | 2.582 | 0 |
| NT2RI2009259 | 0 | 0 | 0 | 68.973 | 0 | 0 | 0 |
| NT2RI2009269 | 0 | 0 | 0 | 0 | 0 | 0 | 2.463 |
| NT2RI2009281 | 16.431 | 0 | 0 | 0 | 0 | 0 | 3.508 |
| NT2RI2009402 | 0 | 0 | 0 | 0 | 0 | 0 | 15.637 |
| NT2RI2009406 | 0 | 0 | 0 | 0 | 0 | 5.198 | 14.027 |
| NT2RI2012350 | 4.673 | 26.870 | 0 | 0 | 10.968 | 0 | 0 |
| NT2RI2015342 | 0 | 0 | 0 | 8.866 | 0 | 2.779 | 3.750 |
| NT2RI2015533 | 0 | 0 | 0 | 0 | 0 | 5.742 | 0 |
| NT2RI2017529 | 0 | 0 | 0 | 0 | 0 | 0 | 2.419 |
| NT2RI3000622 | 0 | 0 | 0 | 0 | 0 | 0 | 21.343 |
| NT2RP6000017 | 0 | 0 | 20.175 | 8.818 | 0 | 2.764 | 0 |
| NT2RP6000072 | 0 | 0 | 0 | 0 | 0 | 2.985 | 0 |
| NT2RP6000078 | 0 | 0 | 0 | 0 | 0 | 1.329 | 0 |
| NT2RP6000109 | 0 | 0 | 0 | 0 | 0 | 7.057 | 0 |
| NT2RP6000110 | 0 | 0 | 0 | 0 | 0 | 10.118 | 6.826 |
| NT2RP6000123 | 0 | 0 | 0 | 4.614 | 10.727 | 6.509 | 2.928 |
| NT2RP7000076 | 0 | 0 | 0 | 0 | 0 | 0 | 6.077 |
| NT2RP7000173 | 0 | 0 | 0 | 0 | 0 | 7.190 | 2.772 |
| NT2RP7000238 | 0 | 0 | 0 | 0 | 0 | 0 | 6.454 |
| NT2RP7000271 | 0 | 0 | 0 | 0 | 0 | 5.603 | 0 |
| NT2RP7000425 | 0 | 0 | 0 | 0 | 0 | 0 | 50.844 |
| NT2RP7000477 | 0 | 0 | 0 | 0 | 0 | 3.655 | 0 |
| NT2RP7000624 | 0 | 0 | 0 | 0 | 0 | 8.060 | 0 |
| NT2RP7000906 | 0 | 0 | 0 | 0 | 0 | 0 | 3.252 |
| NT2RP7001166 | 8.732 | 0 | 0 | 0 | 0 | 0 | 0 |
| NT2RP7001231 | 0 | 0 | 0 | 0 | 0 | 38.148 | 0 |
| NT2RP7001283 | 0 | 0 | 0 | 0 | 15.424 | 0 | 0 |
| NT2RP7001591 | 0 | 0 | 0 | 0 | 0 | 0 | 6.481 |
| NT2RP7002028 | 0 | 0 | 0 | 0 | 0 | 0 | 5.010 |
| NT2RP7002379 | 0 | 0 | 0 | 2.967 | 0 | 6.510 | 0 |
| NT2RP7002738 | 0 | 0 | 0 | 0 | 0 | 10.635 | 0 |
| NT2RP7002802 | 0 | 0 | 0 | 0 | 0 | 6.751 | 0 |
| NT2RP7002875 | 0 | 0 | 0 | 0 | 0 | 11.933 | 20.703 |
| NT2RP7003203 | 0 | 0 | 0 | 0 | 0 | 13.650 | 9.209 |
| NT2RP7003261 | 0 | 0 | 0 | 0 | 0 | 2.510 | 0 |
| NT2RP7003319 | 0 | 0 | 0 | 0 | 0 | 0 | 2.274 |
| NT2RP7003511 | 0 | 0 | 0 | 19.474 | 0 | 6.105 | 0 |
| NT2RP7003688 | 0 | 0 | 0 | 0 | 0 | 2.728 | 1.841 |
| NT2RP7003724 | 0 | 0 | 0 | 0 | 0 | 0 | 23.020 |
| NT2RP7003960 | 0 | 0 | 0 | 0 | 0 | 0 | 4.130 |
| NT2RP7004123 | 0 | 0 | 0 | 0 | 0 | 7.090 | 0 |
| NT2RP7004173 | 8.916 | 0 | 0 | 0 | 0 | 2.822 | 3.808 |
| NT2RP7004196 | 0 | 0 | 0 | 0 | 0 | 2.421 | 6.532 |
| NT2RP7004204 | 0 | 0 | 0 | 0 | 0 | 3.150 | 4.251 |
| NT2RP7004348 | 0 | 0 | 0 | 13.318 | 0 | 0 | 5.634 |
| NT2RP7004352 | 0 | 0 | 0 | 0 | 0 | 3.138 | 8.468 |
| NT2RP7004373 | 0 | 0 | 0 | 0 | 0 | 2.174 | 2.934 |
| NT2RP7004481 | 0 | 0 | 0 | 0 | 0 | 9.936 | 0 |
| NT2RP7004728 | 0 | 0 | 0 | 0 | 0 | 5.807 | 7.836 |
| NT2RP7004751 | 0 | 0 | 0 | 2.758 | 0 | 3.459 | 0 |
| NT2RP7004884 | 0 | 0 | 0 | 0 | 0 | 0 | 6.844 |
| NT2RP7004925 | 0 | 0 | 0 | 0 | 0 | 0 | 3.148 |
| NT2RP7004946 | 0 | 0 | 0 | 0 | 0 | 7.952 | 6.438 |
| NT2RP7005513 | 0 | 0 | 0 | 0 | 0 | 3.123 | 4.213 |
| NT2RP7006188 | 0 | 0 | 0 | 0 | 0 | 4.810 | 0 |
| NT2RP7006223 | 0 | 0 | 0 | 0 | 0 | 7.806 | 0 |
| NT2RP7006619 | 0 | 0 | 0 | 0 | 0 | 8.156 | 11.005 |
| NT2RP7006717 | 0 | 0 | 0 | 0 | 0 | 2.431 | 0 |
| NT2RP7006986 | 0 | 0 | 0 | 0 | 0 | 0 | 9.365 |
| NT2RP7007114 | 0 | 0 | 0 | 0 | 0 | 30.267 | 0 |
| NT2RP7007269 | 0 | 0 | 0 | 0 | 0 | 43.392 | 0 |
| NT2RP7007387 | 0 | 0 | 0 | 0 | 0 | 7.584 | 5.117 |
| NT2RP7007530 | 0 | 0 | 0 | 0 | 0 | 12.151 | 0 |
| NT2RP7007842 | 0 | 0 | 0 | 0 | 0 | 3.543 | 4.781 |
| NT2RP7008015 | 0 | 0 | 0 | 0 | 0 | 0 | 2.515 |
| NT2RP7008167 | 0 | 0 | 0 | 5.349 | 0 | 0 | 0 |
| NT2RP7008190 | 0 | 0 | 0 | 7.817 | 0 | 0 | 3.307 |
| NT2RP7008441 | 0 | 0 | 0 | 0 | 0 | 37.192 | 0 |
| NT2RP7008714 | 0 | 0 | 0 | 0 | 0 | 7.165 | 19.335 |
| NT2RP7009030 | 0 | 0 | 0 | 0 | 0 | 17.773 | 0 |
| NT2RP7009087 | 0 | 0 | 0 | 0 | 0 | 8.976 | 0 |
| NT2RP7009097 | 0 | 0 | 0 | 0 | 0 | 3.321 | 0 |
| NT2RP7009108 | 0 | 0 | 0 | 0 | 0 | 0 | 2.216 |
| NT2RP7009225 | 0 | 0 | 0 | 16.577 | 0 | 0 | 0 |
| NT2RP7009259 | 0 | 0 | 0 | 0 | 0 | 2.076 | 0 |
| NT2RP7009394 | 0 | 0 | 0 | 0 | 0 | 6.702 | 0 |
| NT2RP7009481 | 0 | 0 | 0 | 0 | 0 | 0 | 10.209 |
| NT2RP7009482 | 0 | 0 | 0 | 0 | 0 | 0.856 | 1.155 |
| NT2RP7009498 | 0 | 0 | 0 | 0 | 0 | 0 | 2.512 |
| NT2RP7009577 | 0 | 0 | 0 | 0 | 0 | 43.392 | 0 |
| NT2RP7011318 | 0 | 0 | 0 | 0 | 0 | 43.392 | 0 |
| NT2RP7013764 | 0 | 0 | 0 | 0 | 0 | 20.975 | 0 |
| NT2RP7015512 | 0 | 0 | 0 | 0 | 0 | 11.941 | 0 |
| NT2RP7019401 | 0 | 0 | 0 | 0 | 0 | 0 | 25.883 |
| NT2RP8001604 | 0 | 0 | 0 | 0 | 0 | 0 | 17.297 |
| NT2RP8007920 | 0 | 0 | 0 | 0 | 0 | 10.963 | 0 |
| NTONG1000033 | 0 | 0 | 0 | 14.441 | 0 | 2.263 | 0 |
| NTONG1000098 | 0 | 0 | 0 | 3.755 | 17.458 | 1.177 | 0 |
| NTONG1000264 | 0 | 0 | 0 | 0 | 0 | 19.807 | 2.430 |
| NTONG2000265 | 0 | 0 | 0 | 0 | 0 | 0 | 4.250 |
| NTONG2000334 | 0 | 0 | 0 | 0 | 0 | 3.777 | 5.097 |
| NTONG2000878 | 0 | 0 | 0 | 0 | 0 | 4.363 | 0 |
| NTONG2000977 | 11.477 | 0 | 0 | 0 | 0 | 0 | 0 |
| NTONG2000985 | 0 | 0 | 0 | 10.987 | 0 | 3.444 | 0 |
| NTONG2001550 | 0 | 0 | 0 | 0 | 0 | 0 | 8.676 |
| NTONG2001612 | 0 | 0 | 15.137 | 0 | 0 | 2.074 | 0 |
| NTONG2001762 | 0 | 0 | 0 | 0 | 0 | 13.326 | 0 |
| NTONG2004918 | 0 | 0 | 0 | 0 | 0 | 11.964 | 0 |
| NTONG2005520 | 0 | 0 | 0 | 3.923 | 0 | 1.230 | 1.659 |
| NTONG2005897 | 0 | 0 | 0 | 0 | 0 | 0 | 34.223 |
| NTONG2005969 | 0 | 0 | 0 | 0 | 0 | 0 | 8.008 |
| NTONG2006709 | 0 | 0 | 0 | 0 | 0 | 16.742 | 0 |
| NTONG2008483 | 0 | 0 | 0 | 0 | 0 | 1.476 | 1.992 |
| OCBBF1000049 | 0 | 0 | 0 | 0 | 0 | 9.180 | 12.388 |
| OCBBF1000067 | 0 | 0 | 0 | 0 | 0 | 13.507 | 63.788 |
| OCBBF1000080 | 0 | 0 | 0 | 0 | 0 | 35.323 | 0 |
| OCBBF1000104 | 0 | 0 | 0 | 0 | 0 | 38.054 | 0 |
| OCBBF1000114 | 0 | 0 | 0 | 0 | 0 | 0 | 6.355 |
| OCBBF1000146 | 0 | 0 | 0 | 0 | 0 | 12.749 | 17.203 |
| OCBBF1000153 | 0 | 0 | 0 | 0 | 0 | 15.675 | 0 |
| OCBBF1000185 | 0 | 0 | 0 | 0 | 0 | 15.555 | 0 |
| OCBBF1000188 | 0 | 0 | 0 | 0 | 0 | 1.924 | 0 |
| OCBBF2000013 | 0 | 0 | 0 | 0 | 0 | 3.595 | 14.552 |
| OCBBF2000015 | 0 | 0 | 0 | 0 | 0 | 2.923 | 0 |
| OCBBF2000126 | 0 | 0 | 0 | 0 | 0 | 0 | 9.689 |
| OCBBF2000178 | 0 | 0 | 0 | 0 | 0 | 6.043 | 5.436 |
| OCBBF2000277 | 0 | 0 | 0 | 0 | 0 | 13.573 | 0 |
| OCBBF2000323 | 0 | 0 | 0 | 0 | 0 | 28.033 | 37.827 |
| OCBBF2000467 | 0 | 0 | 0 | 0 | 0 | 14.905 | 10.056 |
| OCBBF2000831 | 0 | 0 | 0 | 0 | 0 | 17.790 | 0 |
| OCBBF2000846 | 0 | 0 | 0 | 0 | 0 | 2.704 | 1.824 |
| OCBBF2000904 | 0 | 0 | 0 | 0 | 0 | 22.402 | 10.076 |
| OCBBF2000986 | 0 | 0 | 0 | 0 | 0 | 3.561 | 0 |
| OCBBF2001166 | 0 | 0 | 0 | 0 | 0 | 20.251 | 0 |
| OCBBF2001210 | 0 | 0 | 0 | 0 | 0 | 13.397 | 0 |
| OCBBF2001252 | 0 | 0 | 0 | 0 | 0 | 34.015 | 22.949 |
| OCBBF2001323 | 0 | 0 | 0 | 0 | 0 | 47.657 | 0 |
| OCBBF2001402 | 0 | 0 | 0 | 0 | 0 | 59.793 | 0 |
| OCBBF2001492 | 0 | 0 | 0 | 0 | 0 | 2.535 | 0 |
| OCBBF2001639 | 0 | 0 | 0 | 0 | 0 | 0 | 11.723 |
| OCBBF2001687 | 0 | 0 | 0 | 0 | 0 | 10.661 | 0 |
| OCBBF2001749 | 0 | 0 | 0 | 0 | 0 | 59.793 | 0 |
| OCBBF2001794 | 0 | 0 | 0 | 0 | 0 | 0 | 11.785 |
| OCBBF2002290 | 0 | 0 | 0 | 0 | 0 | 18.632 | 0 |
| OCBBF2002376 | 0 | 0 | 0 | 0 | 0 | 14.065 | 0 |
| OCBBF2002626 | 0 | 0 | 0 | 0 | 0 | 1.560 | 0 |
| OCBBF2002656 | 0 | 0 | 0 | 0 | 0 | 9.168 | 0 |
| OCBBF2002663 | 0 | 0 | 0 | 0 | 0 | 59.793 | 0 |
| OCBBF2003052 | 0 | 0 | 0 | 0 | 0 | 28.068 | 9.469 |
| OCBBF2003246 | 0 | 0 | 0 | 0 | 0 | 0 | 24.396 |
| OCBBF2003518 | 0 | 0 | 0 | 0 | 0 | 0 | 2.570 |
| OCBBF2003925 | 0 | 0 | 0 | 9.821 | 0 | 0 | 2.077 |
| OCBBF2004038 | 0 | 0 | 0 | 0 | 0 | 0 | 66.741 |
| OCBBF2004104 | 0 | 0 | 0 | 0 | 0 | 15.181 | 13.657 |
| OCBBF2004273 | 0 | 0 | 0 | 0 | 0 | 3.312 | 0 |
| OCBBF2004757 | 0 | 0 | 0 | 0 | 0 | 18.314 | 30.890 |
| OCBBF2004930 | 0 | 0 | 0 | 0 | 0 | 0 | 25.873 |
| OCBBF2004984 | 0 | 0 | 0 | 0 | 0 | 44.001 | 0 |
| OCBBF2005428 | 0 | 0 | 0 | 0 | 0 | 6.090 | 0 |
| OCBBF2005956 | 0 | 0 | 0 | 0 | 0 | 5.245 | 0 |
| OCBBF2006113 | 0 | 0 | 0 | 0 | 0 | 48.624 | 0 |
| OCBBF2006172 | 0 | 0 | 0 | 0 | 0 | 0 | 13.071 |
| OCBBF2006313 | 0 | 0 | 0 | 0 | 0 | 0 | 14.234 |
| OCBBF2006764 | 0 | 0 | 0 | 0 | 0 | 28.349 | 0 |
| OCBBF2006849 | 0 | 0 | 0 | 0 | 0 | 3.734 | 0 |
| OCBBF2006859 | 0 | 0 | 0 | 0 | 0 | 0 | 18.676 |
| OCBBF2007028 | 0 | 0 | 0 | 8.137 | 0 | 5.102 | 3.442 |
| OCBBF2007196 | 0 | 0 | 0 | 0 | 0 | 33.142 | 0 |
| OCBBF2007415 | 0 | 0 | 0 | 0 | 0 | 2.959 | 0 |
| OCBBF2007762 | 0 | 0 | 0 | 0 | 0 | 29.049 | 0 |
| OCBBF2007829 | 0 | 0 | 0 | 6.472 | 0 | 3.043 | 2.737 |
| OCBBF2007892 | 0 | 0 | 0 | 0 | 0 | 0 | 21.239 |
| OCBBF2008005 | 0 | 0 | 0 | 0 | 0 | 11.733 | 7.916 |
| OCBBF2008041 | 0 | 0 | 0 | 0 | 0 | 7.723 | 0 |
| OCBBF2008340 | 0 | 0 | 0 | 0 | 0 | 28.522 | 0 |
| OCBBF2008466 | 0 | 0 | 0 | 0 | 0 | 0 | 42.651 |
| OCBBF2008701 | 0 | 0 | 0 | 0 | 0 | 0 | 27.016 |
| OCBBF2008770 | 0 | 0 | 0 | 0 | 0 | 24.703 | 33.334 |
| OCBBF2009301 | 0 | 0 | 0 | 0 | 0 | 0 | 5.167 |
| OCBBF2009391 | 0 | 0 | 0 | 0 | 0 | 59.793 | 0 |
| OCBBF2009424 | 0 | 0 | 0 | 0 | 0 | 13.407 | 0 |
| OCBBF2009772 | 0 | 0 | 0 | 0 | 0 | 4.351 | 0 |
| OCBBF2009836 | 0 | 0 | 0 | 0 | 0 | 7.570 | 10.215 |
| OCBBF2010819 | 0 | 0 | 0 | 0 | 0 | 23.173 | 0 |
| OCBBF2010830 | 0 | 0 | 0 | 0 | 0 | 1.626 | 0 |
| OCBBF2010841 | 0 | 0 | 0 | 0 | 0 | 11.483 | 0 |
| OCBBF2010843 | 0 | 0 | 0 | 0 | 0 | 27.517 | 0 |
| OCBBF2010871 | 0 | 0 | 0 | 0 | 0 | 25.333 | 0 |
| OCBBF2011021 | 0 | 0 | 0 | 0 | 0 | 2.670 | 0 |
| OCBBF2011073 | 0 | 0 | 0 | 0 | 0 | 0 | 25.519 |
| OCBBF2011232 | 0 | 0 | 0 | 0 | 0 | 0 | 19.973 |
| OCBBF2011536 | 0 | 0 | 0 | 0 | 0 | 3.686 | 9.946 |
| OCBBF2011625 | 0 | 0 | 0 | 0 | 0 | 0 | 66.741 |
| OCBBF2011759 | 0 | 0 | 0 | 0 | 0 | 3.453 | 0 |
| OCBBF2011887 | 0 | 0 | 0 | 0 | 0 | 8.688 | 0 |
| OCBBF2012095 | 0 | 0 | 0 | 0 | 0 | 0 | 38.146 |
| OCBBF2012191 | 0 | 0 | 0 | 0 | 0 | 59.793 | 0 |
| OCBBF2012553 | 0 | 0 | 0 | 6.912 | 0 | 4.333 | 11.695 |
| OCBBF2012678 | 0 | 0 | 0 | 0 | 0 | 25.486 | 0 |
| OCBBF2012821 | 0 | 0 | 0 | 0 | 0 | 9.245 | 0 |
| OCBBF2012908 | 0 | 0 | 0 | 0 | 0 | 18.162 | 8.169 |
| OCBBF2013011 | 0 | 0 | 0 | 0 | 0 | 13.949 | 0 |
| OCBBF2013149 | 0 | 0 | 0 | 0 | 0 | 0 | 7.733 |
| OCBBF2013481 | 0 | 0 | 0 | 0 | 0 | 10.100 | 0 |
| OCBBF2013789 | 0 | 0 | 0 | 0 | 0 | 27.881 | 0 |
| OCBBF2013843 | 0 | 0 | 0 | 0 | 0 | 2.961 | 3.996 |
| OCBBF2013926 | 0 | 0 | 0 | 0 | 0 | 22.924 | 0 |
| OCBBF2014064 | 0 | 0 | 0 | 0 | 0 | 2.523 | 10.215 |
| OCBBF2014292 | 0 | 0 | 0 | 0 | 0 | 19.134 | 0 |
| OCBBF2014322 | 0 | 0 | 0 | 0 | 0 | 0 | 51.462 |
| OCBBF2014386 | 0 | 0 | 0 | 0 | 0 | 33.591 | 0 |
| OCBBF2014541 | 0 | 0 | 0 | 0 | 0 | 7.244 | 9.774 |
| OCBBF2014873 | 0 | 0 | 0 | 0 | 0 | 26.202 | 0 |
| OCBBF2014880 | 0 | 0 | 0 | 0 | 0 | 59.793 | 0 |
| OCBBF2014928 | 0 | 0 | 0 | 0 | 0 | 59.793 | 0 |
| OCBBF2015503 | 0 | 0 | 0 | 0 | 0 | 41.591 | 0 |
| OCBBF2015599 | 0 | 0 | 0 | 0 | 0 | 59.793 | 0 |
| OCBBF2015751 | 0 | 0 | 0 | 0 | 0 | 5.235 | 0 |
| OCBBF2016405 | 0 | 0 | 0 | 0 | 0 | 71.253 | 0 |
| OCBBF2016928 | 31.972 | 0 | 0 | 0 | 0 | 5.059 | 0 |
| OCBBF2017035 | 0 | 0 | 0 | 0 | 0 | 4.479 | 6.044 |
| OCBBF2017398 | 0 | 0 | 0 | 0 | 0 | 2.376 | 0 |
| OCBBF2017458 | 0 | 0 | 0 | 0 | 0 | 20.570 | 0 |
| OCBBF2017754 | 0 | 0 | 0 | 0 | 0 | 6.936 | 9.360 |
| OCBBF2017791 | 0 | 0 | 0 | 0 | 0 | 4.733 | 6.386 |
| OCBBF2017888 | 0 | 0 | 0 | 0 | 0 | 31.476 | 0 |
| OCBBF2017893 | 0 | 0 | 0 | 0 | 0 | 0 | 4.324 |
| OCBBF2018012 | 0 | 0 | 0 | 0 | 0 | 5.185 | 0 |
| OCBBF2018234 | 0 | 0 | 0 | 0 | 0 | 23.932 | 0 |
| OCBBF2018707 | 0 | 0 | 0 | 0 | 0 | 0 | 11.213 |
| OCBBF2018873 | 0 | 0 | 0 | 0 | 0 | 5.120 | 0 |
| OCBBF2018956 | 0 | 0 | 0 | 9.974 | 0 | 0 | 0 |
| OCBBF2021286 | 0 | 0 | 0 | 0 | 0 | 0 | 23.004 |
| OCBBF2023162 | 0 | 0 | 0 | 0 | 0 | 18.995 | 0 |
| OCBBF2026368 | 0 | 0 | 0 | 0 | 0 | 0 | 20.599 |
| OCBBF2033295 | 0 | 0 | 0 | 0 | 0 | 19.428 | 0 |
| OCBBF2035226 | 0 | 0 | 0 | 0 | 0 | 1.085 | 0 |
| OCBBF3000323 | 0 | 0 | 0 | 0 | 0 | 0 | 16.582 |
| PEBLM1000174 | 0 | 0 | 0 | 0 | 0 | 8.835 | 0 |
| PEBLM2000308 | 0 | 0 | 0 | 0 | 0 | 18.312 | 0 |
| PEBLM2000395 | 10.626 | 0 | 0 | 0 | 0 | 5.044 | 0 |
| PEBLM2003914 | 0 | 0 | 0 | 12.522 | 0 | 0 | 0 |
| PEBLM2006366 | 32.312 | 0 | 0 | 0 | 0 | 0 | 0 |
| PEBLM2007296 | 0 | 0 | 0 | 0 | 47.388 | 0 | 4.311 |
| PEBLM2007774 | 7.033 | 20.220 | 0 | 1.775 | 0 | 0 | 0 |
| PLACE5000113 | 0 | 0 | 0 | 0 | 0 | 6.357 | 0 |
| PLACE5000159 | 0 | 0 | 0 | 0 | 0 | 16.795 | 0 |
| PLACE5000170 | 0 | 0 | 0 | 0 | 0 | 6.423 | 0 |
| PLACE5000172 | 0 | 0 | 0 | 0 | 0 | 0 | 3.240 |
| PLACE6000145 | 12.099 | 0 | 0 | 0 | 0 | 0 | 0 |
| PLACE6000263 | 0 | 0 | 0 | 8.960 | 0 | 0 | 0 |
| PLACE6000379 | 0 | 0 | 0 | 1.041 | 0 | 3.263 | 6.164 |
| PLACE6000463 | 0 | 0 | 0 | 0 | 0 | 14.415 | 0 |
| PLACE6001064 | 0 | 0 | 0 | 3.381 | 0 | 4.240 | 0 |
| PLACE6001262 | 0 | 0 | 0 | 0 | 0 | 0 | 21.783 |
| PLACE6001281 | 0 | 0 | 0 | 0 | 0 | 0 | 8.582 |
| PLACE6001443 | 0 | 0 | 0 | 11.576 | 0 | 0 | 0 |
| PLACE6002084 | 4.312 | 0 | 4.980 | 4.353 | 0 | 0.682 | 0 |
| PLACE6002102 | 0 | 0 | 0 | 0 | 0 | 5.218 | 0 |
| PLACE6004005 | 0 | 0 | 0 | 0 | 0 | 5.472 | 29.535 |
| PLACE6004312 | 0 | 0 | 8.132 | 3.554 | 0 | 1.114 | 1.503 |
| PLACE6004931 | 76.447 | 0 | 0 | 0 | 0 | 0 | 0 |
| PLACE6007436 | 0 | 0 | 0 | 0 | 0 | 6.637 | 8.956 |
| PLACE6008775 | 0 | 0 | 0 | 0 | 0 | 0 | 4.595 |
| PLACE6010077 | 0 | 0 | 11.740 | 5.131 | 0 | 0 | 2.170 |
| PLACE6010848 | 0 | 0 | 0 | 0 | 0 | 15.118 | 0 |
| PLACE6011041 | 0 | 0 | 0 | 0 | 0 | 0 | 6.782 |
| PLACE6011057 | 0 | 0 | 30.391 | 0 | 0 | 0 | 0 |
| PLACE6013222 | 0 | 0 | 0 | 0 | 0 | 27.309 | 0 |
| PLACE6015051 | 0 | 0 | 0 | 0 | 0 | 8.516 | 0 |
| PLACE6016210 | 0 | 0 | 0 | 0 | 0 | 0 | 31.569 |
| PLACE6016942 | 0 | 0 | 0 | 0 | 0 | 0 | 9.752 |
| PLACE6018031 | 0 | 0 | 0 | 0 | 0 | 0 | 16.761 |
| PROST1000097 | 0 | 0 | 0 | 0 | 0 | 0 | 1.466 |
| PROST1000120 | 0 | 0 | 0 | 0 | 0 | 6.092 | 0 |
| PROST1000199 | 8.530 | 0 | 0 | 0 | 0 | 1.350 | 0 |
| PROST1000215 | 0 | 0 | 0 | 0 | 0 | 9.449 | 0 |
| PROST1000272 | 0 | 0 | 0 | 0 | 0 | 0 | 15.731 |
| PROST1000526 | 0 | 0 | 0 | 0 | 0 | 4.668 | 0 |
| PROST2000079 | 0 | 0 | 0 | 0 | 0 | 5.530 | 0 |
| PROST2000138 | 0 | 0 | 0 | 0 | 0 | 0.160 | 0 |
| PROST2000165 | 0 | 0 | 0 | 0 | 0 | 50.609 | 0 |
| PROST2000241 | 71.444 | 0 | 0 | 0 | 0 | 0 | 0 |
| PROST2000337 | 0 | 0 | 0 | 0 | 0 | 34.251 | 0 |
| PROST2000893 | 0 | 0 | 0 | 0 | 0 | 0 | 41.278 |
| PROST2001180 | 0 | 0 | 0 | 0 | 0 | 4.587 | 6.190 |
| PROST2001213 | 0 | 0 | 0 | 0 | 0 | 0 | 20.781 |
| PROST2001823 | 0 | 0 | 0 | 0 | 0 | 34.251 | 0 |
| PROST2001883 | 0 | 59.960 | 0 | 0 | 0 | 0 | 0 |
| PROST2002078 | 0 | 0 | 0 | 0 | 66.593 | 0 | 0 |
| PROST2003338 | 0 | 0 | 0 | 0 | 0 | 4.136 | 5.581 |
| PROST2004095 | 0 | 0 | 0 | 2.758 | 0 | 5.188 | 1.167 |
| PROST2004258 | 0 | 0 | 0 | 22.485 | 0 | 0 | 9.511 |
| PROST2004270 | 0 | 0 | 0 | 0 | 0 | 2.173 | 0 |
| PROST2004416 | 0 | 0 | 0 | 0 | 0 | 0 | 1.695 |
| PROST2004570 | 0 | 0 | 0 | 0 | 0 | 13.848 | 0 |
| PROST2005039 | 0 | 0 | 0 | 0 | 0 | 25.460 | 0 |
| PROST2005143 | 0 | 0 | 0 | 0 | 0 | 12.533 | 0 |
| PROST2005793 | 0 | 0 | 0 | 0 | 0 | 7.245 | 0 |
| PROST2006030 | 0 | 0 | 0 | 0 | 0 | 6.990 | 0 |
| PROST2006536 | 0 | 0 | 0 | 0 | 0 | 16.398 | 22.127 |
| PROST2007612 | 0 | 0 | 0 | 0 | 0 | 11.799 | 0 |
| PROST2008268 | 0 | 0 | 0 | 0 | 0 | 34.251 | 0 |
| PROST2008271 | 0 | 0 | 0 | 0 | 0 | 7.240 | 0 |
| PROST2008360 | 0 | 0 | 0 | 0 | 0 | 3.325 | 0 |
| PROST2008770 | 0 | 0 | 0 | 0 | 0 | 0 | 1.945 |
| PROST2010382 | 0 | 0 | 0 | 0 | 85.749 | 0 | 0 |
| PROST2010782 | 0 | 0 | 3.279 | 0 | 0 | 0 | 0 |
| PROST2011038 | 0 | 0 | 0 | 0 | 0 | 0 | 29.914 |
| PROST2011482 | 0 | 0 | 0 | 0 | 0 | 34.251 | 0 |
| PROST2011577 | 0 | 0 | 0 | 0 | 0 | 5.703 | 0 |
| PROST2012007 | 0 | 0 | 0 | 0 | 0 | 2.097 | 0 |
| PROST2012448 | 0 | 0 | 0 | 0 | 0 | 15.073 | 0 |
| PROST2015332 | 0 | 0 | 0 | 0 | 0 | 0 | 6.425 |
| PROST2017529 | 0 | 0 | 0 | 14.958 | 0 | 0 | 0 |
| PROST2018977 | 0 | 0 | 0 | 0 | 0 | 24.007 | 0 |
| PROST2019253 | 0 | 0 | 0 | 0 | 0 | 52.767 | 20.942 |
| PUAEN1000057 | 0 | 0 | 0 | 0 | 0 | 3.793 | 0 |
| PUAEN1000164 | 4.647 | 0 | 0 | 0 | 0 | 0 | 0.992 |
| PUAEN1000239 | 0 | 0 | 0 | 0 | 0 | 5.727 | 0 |
| PUAEN2000312 | 0 | 0 | 0 | 0 | 0 | 16.356 | 0 |
| PUAEN2000374 | 0 | 0 | 0 | 1.886 | 8.770 | 0 | 0 |
| PUAEN2000535 | 0 | 0 | 0 | 0 | 0 | 1.486 | 0 |
| PUAEN2000942 | 0 | 0 | 0 | 0 | 0 | 24.665 | 0 |
| PUAEN2001345 | 0 | 0 | 0 | 0 | 80.232 | 0 | 0 |
| PUAEN2002489 | 0 | 0 | 0 | 0 | 0 | 24.108 | 0 |
| PUAEN2003408 | 0 | 0 | 40.610 | 0 | 0 | 5.564 | 15.015 |
| PUAEN2007044 | 0 | 0 | 0 | 16.894 | 0 | 0 | 0 |
| PUAEN2009655 | 0 | 0 | 49.285 | 0 | 0 | 0 | 0 |
| RECTM1000051 | 0 | 0 | 31.123 | 0 | 0 | 0 | 0 |
| RECTM2000349 | 0 | 0 | 0 | 0 | 0 | 7.826 | 0 |
| SALGL1000157 | 0 | 8.730 | 0 | 1.533 | 0 | 0 | 0.324 |
| SKMUS1000022 | 0 | 0 | 0 | 0 | 11.405 | 0 | 0 |
| SKMUS1000084 | 0 | 0 | 0 | 0 | 0 | 2.845 | 0 |
| SKMUS1000186 | 0 | 0 | 0 | 0 | 0 | 0.742 | 0 |
| SKMUS2000020 | 0 | 0 | 0 | 0 | 0 | 0 | 9.182 |
| SKMUS2000074 | 0 | 0 | 0 | 0 | 0 | 9.448 | 0 |
| SKMUS2000361 | 0 | 0 | 0 | 6.302 | 0 | 0 | 2.666 |
| SKMUS2000380 | 0 | 0 | 0 | 0 | 0 | 0 | 5.406 |
| SKMUS2000484 | 0 | 0 | 0 | 0 | 0 | 0 | 8.755 |
| SKMUS2000690 | 0 | 0 | 65.679 | 0 | 0 | 0 | 0 |
| SKMUS2000902 | 0 | 0 | 0 | 31.874 | 0 | 0 | 0 |
| SKMUS2000931 | 0 | 0 | 0 | 6.414 | 0 | 0 | 1.356 |
| SKMUS2001147 | 0 | 0 | 0 | 0 | 0 | 5.121 | 0 |
| SKMUS2001402 | 0 | 0 | 0 | 0 | 0 | 0 | 10.509 |
| SKMUS2001454 | 0 | 0 | 0 | 0 | 0 | 2.494 | 0.841 |
| SKNMC1000110 | 0 | 0 | 0 | 0 | 0 | 6.689 | 0 |
| SKNMC1000168 | 0 | 0 | 0 | 0 | 22.946 | 0 | 2.088 |
| SKNMC1000229 | 0 | 0 | 0 | 0 | 0 | 9.712 | 0 |
| SKNMC2000065 | 0 | 0 | 0 | 6.362 | 0 | 7.977 | 5.382 |
| SKNMC2000097 | 0 | 0 | 0 | 0 | 0 | 0.823 | 1.111 |
| SKNMC2000305 | 0 | 0 | 0 | 0 | 0 | 0 | 8.781 |
| SKNMC2000356 | 0 | 0 | 0 | 0 | 0 | 16.334 | 0 |
| SKNMC2000635 | 0 | 0 | 0 | 4.228 | 0 | 0 | 0 |
| SKNMC2000649 | 0 | 0 | 0 | 5.377 | 0 | 1.685 | 0 |
| SKNMC2000698 | 0 | 0 | 0 | 0 | 0 | 3.816 | 0 |
| SKNMC2000877 | 0 | 0 | 0 | 0 | 0 | 2.189 | 2.954 |
| SKNSH1000002 | 0 | 0 | 0 | 8.471 | 0 | 0 | 0 |
| SKNSH2000163 | 0 | 0 | 0 | 0 | 0 | 19.605 | 0 |
| SKNSH2000347 | 0 | 0 | 0 | 26.446 | 0 | 0 | 0 |
| SKNSH2000716 | 0 | 0 | 0 | 0 | 0 | 6.404 | 4.320 |
| SKNSH2000819 | 0 | 0 | 0 | 0 | 0 | 4.598 | 0 |
| SKNSH2003528 | 0 | 0 | 40.935 | 0 | 0 | 5.608 | 0 |
| SKNSH2006234 | 0 | 0 | 0 | 20.629 | 0 | 0 | 0 |
| SKNSH2008940 | 0 | 0 | 25.857 | 0 | 0 | 0 | 0 |
| SKNSH2009357 | 0 | 0 | 0 | 0 | 0 | 0 | 23.814 |
| SMINT1000117 | 0 | 0 | 0 | 0 | 0 | 7.290 | 0 |
| SMINT2000007 | 0 | 0 | 0 | 0 | 0 | 0 | 14.994 |
| SMINT2000073 | 0 | 0 | 0 | 0 | 0 | 32.054 | 0 |
| SMINT2000396 | 0 | 0 | 0 | 21.642 | 0 | 6.784 | 0 |
| SMINT2000441 | 0 | 0 | 0 | 2.660 | 0 | 0.834 | 2.250 |
| SMINT2000468 | 0 | 0 | 0 | 0 | 0 | 0 | 5.704 |
| SMINT2000602 | 6.417 | 0 | 7.412 | 0 | 0 | 0 | 0 |
| SMINT2000629 | 0 | 0 | 0 | 0 | 0 | 4.961 | 6.695 |
| SMINT2000694 | 0 | 0 | 0 | 0 | 0 | 0 | 2.963 |
| SMINT2002159 | 0 | 0 | 0 | 40.407 | 0 | 0 | 0 |
| SMINT2002620 | 0 | 0 | 0 | 0 | 0 | 4.186 | 0 |
| SMINT2002689 | 0 | 0 | 0 | 0 | 0 | 0 | 2.930 |
| SMINT2002884 | 43.124 | 0 | 0 | 0 | 0 | 0 | 0 |
| SMINT2003074 | 0 | 0 | 0 | 0 | 0 | 7.597 | 0 |
| SMINT2003169 | 5.491 | 0 | 0 | 0 | 0 | 0 | 0 |
| SMINT2003340 | 0 | 0 | 39.619 | 0 | 0 | 0 | 7.324 |
| SMINT2003505 | 36.765 | 0 | 0 | 0 | 0 | 0 | 0 |
| SMINT2004729 | 0 | 0 | 0 | 0 | 0 | 23.727 | 0 |
| SMINT2004872 | 0 | 0 | 0 | 12.464 | 0 | 0 | 0 |
| SMINT2004972 | 0 | 0 | 0 | 46.681 | 0 | 0 | 19.746 |
| SMINT2005368 | 0 | 0 | 0 | 0 | 0 | 2.170 | 0 |
| SMINT2006716 | 0 | 0 | 0 | 0 | 0 | 0 | 58.556 |
| SMINT2007508 | 0 | 0 | 0 | 0 | 0 | 0 | 41.399 |
| SMINT2008921 | 0 | 0 | 0 | 0 | 0 | 2.050 | 0 |
| SMINT2008960 | 0 | 0 | 9.225 | 0 | 0 | 1.264 | 1.705 |
| SMINT2010084 | 0 | 0 | 0 | 0 | 0 | 0 | 8.566 |
| SMINT2010426 | 0 | 0 | 0 | 31.925 | 0 | 0 | 0 |
| SMINT2010901 | 0 | 0 | 0 | 0 | 0 | 6.488 | 0 |
| SMINT2011588 | 0 | 0 | 0 | 0 | 0 | 34.363 | 0 |
| SMINT2018681 | 48.822 | 0 | 0 | 0 | 0 | 0 | 0 |
| SPLEN1000049 | 0 | 0 | 0 | 0 | 0 | 40.275 | 0 |
| SPLEN1000089 | 0 | 0 | 40.187 | 0 | 27.222 | 0 | 0 |
| SPLEN1000106 | 0 | 0 | 0 | 0 | 0 | 4.300 | 0 |
| SPLEN1000116 | 0 | 0 | 0 | 0 | 13.562 | 0 | 1.234 |
| SPLEN1000166 | 0 | 0 | 0 | 0 | 0 | 12.530 | 2.818 |
| SPLEN2000255 | 0 | 0 | 0 | 0 | 0 | 2.837 | 3.828 |
| SPLEN2000267 | 0 | 0 | 0 | 0 | 0 | 0 | 14.553 |
| SPLEN2000348 | 0 | 0 | 0 | 0 | 0 | 0 | 6.974 |
| SPLEN2000505 | 0 | 0 | 5.683 | 2.484 | 0 | 1.557 | 0 |
| SPLEN2000698 | 0 | 0 | 28.593 | 0 | 0 | 0 | 0 |
| SPLEN2000882 | 0 | 0 | 0 | 10.353 | 0 | 0 | 0 |
| SPLEN2001599 | 0 | 0 | 0 | 0 | 0 | 0 | 17.788 |
| SPLEN2001689 | 0 | 0 | 0 | 0 | 0 | 0 | 34.793 |
| SPLEN2002007 | 0 | 0 | 0 | 0 | 0 | 0 | 47.573 |
| SPLEN2002335 | 0 | 0 | 0 | 0 | 27.746 | 3.741 | 2.524 |
| SPLEN2002467 | 0 | 0 | 0 | 0 | 0 | 6.019 | 0 |
| SPLEN2004078 | 0 | 0 | 0 | 0 | 0 | 27.607 | 0 |
| SPLEN2004555 | 0 | 0 | 0 | 0 | 0 | 3.933 | 2.653 |
| SPLEN2004773 | 16.618 | 0 | 9.597 | 0 | 0 | 2.630 | 3.548 |
| SPLEN2005009 | 0 | 0 | 0 | 0 | 0 | 15.882 | 0 |
| SPLEN2005342 | 0 | 0 | 0 | 0 | 0 | 19.711 | 13.299 |
| SPLEN2005869 | 74.977 | 0 | 0 | 0 | 0 | 0 | 0 |
| SPLEN2006122 | 0 | 0 | 0 | 0 | 23.078 | 0 | 2.099 |
| SPLEN2006133 | 0 | 0 | 0 | 0 | 0 | 1.753 | 0 |
| SPLEN2006143 | 0 | 0 | 0 | 0 | 0 | 4.133 | 0 |
| SPLEN2006232 | 33.489 | 0 | 0 | 0 | 0 | 0 | 0 |
| SPLEN2006305 | 0 | 0 | 0 | 0 | 17.309 | 1.167 | 0 |
| SPLEN2006374 | 0 | 0 | 0 | 0 | 0 | 2.808 | 0 |
| SPLEN2007276 | 0 | 0 | 35.761 | 0 | 0 | 0 | 0 |
| SPLEN2007498 | 0 | 0 | 0 | 0 | 0 | 4.835 | 19.572 |
| SPLEN2007951 | 0 | 0 | 0 | 0 | 0 | 0 | 58.898 |
| SPLEN2008460 | 30.589 | 0 | 0 | 0 | 0 | 0 | 0 |
| SPLEN2009081 | 0 | 0 | 0 | 0 | 0 | 0 | 16.134 |
| SPLEN2010195 | 4.529 | 0 | 5.231 | 2.286 | 0 | 0 | 0 |
| SPLEN2011419 | 0 | 0 | 0 | 14.082 | 0 | 0 | 0 |
| SPLEN2012571 | 0 | 0 | 0 | 0 | 0 | 0 | 7.925 |
| SPLEN2013821 | 87.032 | 0 | 0 | 0 | 0 | 0 | 0 |
| SPLEN2013936 | 0 | 0 | 0 | 0 | 0 | 12.327 | 0 |
| SPLEN2014080 | 55.315 | 0 | 0 | 0 | 0 | 0 | 0 |
| SPLEN2014381 | 0 | 0 | 0 | 0 | 0 | 0 | 74.133 |
| SPLEN2015310 | 0 | 0 | 0 | 0 | 0 | 0 | 1.429 |
| SPLEN2016139 | 29.917 | 0 | 0 | 0 | 0 | 0 | 0 |
| SPLEN2016268 | 0 | 0 | 0 | 2.546 | 0 | 3.991 | 0 |
| SPLEN2016972 | 0 | 0 | 0 | 0 | 0 | 3.746 | 0 |
| SPLEN2017318 | 0 | 0 | 0 | 0 | 0 | 9.374 | 0 |
| SPLEN2018157 | 0 | 0 | 0 | 0 | 0 | 13.368 | 0 |
| SPLEN2018159 | 0 | 0 | 0 | 55.812 | 0 | 0 | 0 |
| SPLEN2019169 | 0 | 0 | 0 | 0 | 0 | 10.459 | 0 |
| SPLEN2019405 | 0 | 0 | 0 | 0 | 0 | 2.646 | 0 |
| SPLEN2019505 | 0 | 0 | 66.106 | 0 | 0 | 9.057 | 12.221 |
| SPLEN2021194 | 0 | 0 | 0 | 0 | 0 | 12.464 | 0 |
| SPLEN2027113 | 0 | 0 | 0 | 9.643 | 0 | 0 | 0 |
| SPLEN2028365 | 0 | 0 | 0 | 0 | 94.03 | 0 | 0 |
| SPLEN2028424 | 0 | 0 | 0 | 0 | 0 | 51.503 | 0 |
| SPLEN2028466 | 0 | 0 | 0 | 0 | 0 | 0 | 1.031 |
| SPLEN2032157 | 0 | 0 | 0 | 0 | 0 | 0 | 14.245 |
| SPLEN2036932 | 0 | 0 | 0 | 0 | 0 | 0 | 21.926 |
| SPLEN2039379 | 0 | 0 | 0 | 0 | 0 | 0 | 7.220 |
| STOMA1000189 | 0 | 0 | 0 | 9.216 | 0 | 0 | 0 |
| STOMA2000395 | 0 | 0 | 0 | 0 | 0 | 0 | 5.322 |
| SYNOV1000124 | 0 | 0 | 0 | 0 | 0 | 6.166 | 1.387 |
| SYNOV2001033 | 0 | 0 | 0 | 3.533 | 16.429 | 0 | 0 |
| SYNOV2001144 | 0 | 0 | 19.072 | 0 | 0 | 0 | 0 |
| SYNOV2001262 | 0 | 0 | 0 | 0 | 0 | 3.854 | 0 |
| SYNOV2001374 | 0 | 0 | 0 | 0 | 0 | 3.384 | 0 |
| SYNOV2008765 | 0 | 96.887 | 0 | 0 | 0 | 0 | 0 |
| SYNOV2009172 | 0 | 0 | 0 | 0 | 14.260 | 0 | 0 |
| SYNOV4007671 | 0 | 0 | 0 | 4.320 | 0 | 1.354 | 1.827 |
| SYNOV4009129 | 0 | 0 | 0 | 0 | 0 | 28.685 | 19.353 |
| T1ESE2000609 | 0 | 0 | 0 | 26.790 | 0 | 0 | 5.666 |
| T1ESE2000904 | 0 | 0 | 0 | 13.606 | 0 | 0 | 5.755 |
| TBAES2009387 | 0 | 0 | 0 | 0 | 0 | 0.988 | 0 |
| TCOLN1000180 | 0 | 0 | 0 | 0 | 0 | 4.523 | 0 |
| TESOP2002273 | 0 | 0 | 0 | 0 | 0 | 7.498 | 0 |
| TESOP2002451 | 0 | 0 | 0 | 0 | 0 | 4.388 | 0 |
| TESOP2007912 | 0 | 0 | 0 | 0 | 0 | 21.261 | 0 |
| TESTI1000023 | 0 | 0 | 0 | 0 | 0 | 0 | 8.362 |
| TESTI2000100 | 0 | 0 | 0 | 0 | 0 | 0 | 48.295 |
| TESTI2000184 | 0 | 0 | 0 | 0 | 0 | 4.177 | 0 |
| TESTI2000238 | 0 | 0 | 0 | 0 | 0 | 58.560 | 0 |
| TESTI2000356 | 0 | 0 | 0 | 0 | 0 | 0 | 43.714 |
| TESTI2001099 | 0 | 0 | 0 | 2.888 | 0 | 3.621 | 2.443 |
| TESTI2001141 | 0 | 0 | 0 | 0 | 0 | 17.445 | 23.540 |
| TESTI2001269 | 0 | 0 | 0 | 3.217 | 0 | 0 | 0 |
| TESTI2001511 | 0 | 0 | 0 | 0 | 47.942 | 0 | 0 |
| TESTI2002149 | 0 | 0 | 0 | 0 | 0 | 6.180 | 0 |
| TESTI2002840 | 0 | 0 | 0 | 0 | 0 | 73.865 | 0 |
| TESTI2003061 | 0 | 0 | 0 | 0 | 0 | 0 | 32.281 |
| TESTI2003413 | 0 | 0 | 0 | 15.344 | 0 | 4.810 | 0 |
| TESTI2003454 | 61.996 | 0 | 0 | 0 | 0 | 0 | 0 |
| TESTI2003533 | 0 | 0 | 0 | 0 | 0 | 1.602 | 2.161 |
| TESTI2004031 | 4.680 | 0 | 0 | 2.363 | 0 | 0.741 | 0 |
| TESTI2004153 | 0 | 0 | 0 | 0 | 0 | 0 | 10.815 |
| TESTI2004295 | 0 | 0 | 0 | 0 | 0 | 0 | 20.413 |
| TESTI2005040 | 0 | 0 | 0 | 0 | 0 | 58.560 | 0 |
| TESTI2005408 | 0 | 0 | 0 | 10.441 | 0 | 0 | 0 |
| TESTI2005564 | 0 | 0 | 0 | 0 | 0 | 0 | 9.129 |
| TESTI2005784 | 0 | 0 | 0 | 17.528 | 0 | 0 | 0 |
| TESTI2005908 | 0 | 0 | 0 | 0 | 0 | 0 | 3.925 |
| TESTI2005981 | 0 | 0 | 0 | 0 | 0 | 57.537 | 0 |
| TESTI2006051 | 0 | 0 | 5.710 | 0 | 0 | 0 | 0 |
| TESTI2006120 | 0 | 0 | 0 | 0 | 0 | 0 | 12.098 |
| TESTI2006453 | 0 | 56.495 | 0 | 0 | 0 | 0 | 0 |
| TESTI2006588 | 0 | 0 | 0 | 0 | 0 | 4.001 | 6.748 |
| TESTI2006748 | 0 | 0 | 0 | 0 | 0 | 12.710 | 8.575 |
| TESTI2006877 | 0 | 0 | 0 | 0 | 0 | 0 | 33.083 |
| TESTI2007407 | 0 | 0 | 0 | 0 | 0 | 5.671 | 0 |
| TESTI2007464 | 0 | 0 | 0 | 0 | 0 | 7.410 | 0 |
| TESTI2007998 | 0 | 0 | 0 | 0 | 0 | 5.842 | 7.883 |
| TESTI2008020 | 0 | 0 | 0 | 0 | 0 | 7.085 | 0 |
| TESTI2008240 | 0 | 0 | 0 | 0 | 0 | 0 | 4.364 |
| TESTI2008394 | 0 | 0 | 0 | 0 | 0 | 73.865 | 0 |
| TESTI2008405 | 60.795 | 0 | 0 | 0 | 0 | 0 | 0 |
| TESTI2009402 | 0 | 0 | 0 | 0 | 0 | 0 | 22.853 |
| TESTI2009520 | 0 | 0 | 0 | 0 | 0 | 0 | 1.870 |
| TESTI2009785 | 0 | 0 | 0 | 0 | 0 | 7.952 | 0 |
| TESTI2009987 | 0 | 0 | 0 | 30.062 | 0 | 0 | 0 |
| TESTI2010513 | 0 | 0 | 0 | 0 | 0 | 0.726 | 1.959 |
| TESTI2010617 | 0 | 0 | 0 | 0 | 0 | 0 | 34.719 |
| TESTI2011680 | 0 | 0 | 0 | 0 | 0 | 27.640 | 0 |
| TESTI2013231 | 0 | 0 | 0 | 0 | 0 | 3.676 | 4.960 |
| TESTI2013264 | 0 | 0 | 0 | 0 | 0 | 0 | 62.629 |
| TESTI2013295 | 0 | 0 | 0 | 0 | 0 | 4.921 | 0 |
| TESTI2014505 | 0 | 0 | 0 | 0 | 0 | 36.406 | 0 |
| TESTI2014780 | 0 | 0 | 0 | 0 | 0 | 52.464 | 0 |
| TESTI2015213 | 0 | 0 | 0 | 0 | 14.063 | 1.896 | 2.559 |
| TESTI2015335 | 0 | 0 | 0 | 0 | 0 | 0 | 55.970 |
| TESTI2015626 | 0 | 0 | 4.435 | 0 | 0 | 0.608 | 0 |
| TESTI2018014 | 0 | 0 | 0 | 0 | 0 | 0 | 9.609 |
| TESTI2018462 | 0 | 0 | 0 | 0 | 0 | 5.221 | 0 |
| TESTI2020510 | 0 | 0 | 0 | 0 | 0 | 13.364 | 0 |
| TESTI2021124 | 0 | 0 | 0 | 0 | 0 | 10.559 | 0 |
| TESTI2021315 | 0 | 0 | 0 | 0 | 0 | 37.439 | 0 |
| TESTI2024864 | 0 | 0 | 0 | 0 | 0 | 51.017 | 0 |
| TESTI2025454 | 0 | 0 | 0 | 0 | 0 | 20.808 | 0 |
| TESTI2026525 | 0 | 0 | 0 | 0 | 0 | 16.076 | 0 |
| TESTI2027496 | 0 | 0 | 0 | 0 | 0 | 17.357 | 0 |
| TESTI2034940 | 0 | 0 | 0 | 0 | 0 | 0.481 | 1.299 |
| TESTI2036969 | 0 | 0 | 0 | 21.174 | 0 | 19.913 | 0 |
| TESTI2046456 | 0 | 0 | 6.242 | 2.728 | 0 | 0.855 | 2.308 |
| TESTI2053723 | 0 | 0 | 0 | 0 | 0 | 0 | 22.570 |
| TESTI4000014 | 0 | 0 | 0 | 0 | 9.787 | 1.980 | 0.890 |
| TESTI4002072 | 0 | 0 | 0 | 0 | 0 | 6.169 | 0 |
| TESTI4002799 | 0 | 0 | 0 | 0 | 12.102 | 0 | 0 |
| TESTI4003703 | 0 | 0 | 0 | 0 | 0 | 8.175 | 22.061 |
| TESTI4004626 | 0 | 0 | 0 | 0 | 0 | 0 | 41.040 |
| TESTI4005857 | 0 | 0 | 0 | 0 | 0 | 0 | 79.225 |
| TESTI4009286 | 0 | 0 | 0 | 0 | 39.728 | 1.339 | 0 |
| TESTI4009454 | 0 | 0 | 0 | 0 | 0 | 0 | 10.019 |
| TESTI4010851 | 0 | 0 | 7.372 | 0 | 0 | 5.050 | 6.814 |
| TESTI4010979 | 0 | 0 | 0 | 0 | 0 | 0 | 48.982 |
| TESTI4013441 | 1.831 | 0 | 0 | 1.849 | 0 | 0.290 | 0.391 |
| TESTI4014175 | 0 | 0 | 0 | 0 | 0 | 49.352 | 0 |
| TESTI4015204 | 0 | 0 | 0 | 0 | 0 | 48.509 | 0 |
| TESTI4019843 | 0 | 0 | 0 | 0 | 0 | 73.865 | 0 |
| TESTI4037156 | 0 | 0 | 0 | 0 | 0 | 2.556 | 1.380 |
| TESTI4043140 | 0 | 0 | 0 | 0 | 0 | 5.123 | 0 |
| THYMU1000002 | 0 | 0 | 0 | 3.488 | 0 | 3.280 | 1.476 |
| THYMU1000016 | 0 | 0 | 10.422 | 0 | 0 | 0.714 | 0 |
| THYMU1000017 | 0 | 0 | 0 | 0 | 0 | 0 | 74.935 |
| THYMU1000041 | 0 | 0 | 0 | 0 | 0 | 4.393 | 0 |
| THYMU1000103 | 0 | 0 | 0 | 0 | 0 | 0 | 4.395 |
| THYMU1000144 | 0 | 0 | 0 | 0 | 0 | 11.947 | 19.344 |
| THYMU1000359 | 6.258 | 0 | 7.228 | 0 | 0 | 0 | 0 |
| THYMU1000374 | 0 | 0 | 0 | 0 | 0 | 0 | 3.734 |
| THYMU1000490 | 0 | 0 | 0 | 0 | 0 | 21.187 | 0 |
| THYMU1000499 | 0 | 0 | 0 | 0 | 0 | 0 | 14.228 |
| THYMU2000382 | 0 | 0 | 0 | 0 | 0 | 0 | 3.022 |
| THYMU2000436 | 0 | 0 | 0 | 0 | 0 | 0 | 2.297 |
| THYMU2000672 | 0 | 0 | 0 | 0 | 0 | 41.789 | 0 |
| THYMU2000684 | 0 | 0 | 0 | 0 | 0 | 0 | 10.379 |
| THYMU2000946 | 0 | 0 | 0 | 0 | 0 | 0 | 3.607 |
| THYMU2001926 | 0 | 0 | 0 | 0 | 0 | 35.857 | 0 |
| THYMU2002162 | 0 | 0 | 0 | 0 | 0 | 20.141 | 27.178 |
| THYMU2002450 | 0 | 0 | 0 | 0 | 0 | 39.856 | 0 |
| THYMU2002583 | 12.101 | 0 | 0 | 3.054 | 0 | 1.915 | 0 |
| THYMU2002756 | 0 | 0 | 0 | 0 | 0 | 0 | 18.250 |
| THYMU2002841 | 0 | 0 | 0 | 17.103 | 0 | 5.362 | 0 |
| THYMU2002983 | 34.939 | 0 | 0 | 0 | 0 | 0 | 0 |
| THYMU2003012 | 10.231 | 0 | 0 | 10.329 | 0 | 0 | 0 |
| THYMU2003419 | 0 | 0 | 0 | 0 | 0 | 0 | 2.504 |
| THYMU2003446 | 0 | 0 | 0 | 0 | 0 | 4.406 | 5.946 |
| THYMU2003542 | 0 | 0 | 0 | 0 | 0 | 3.992 | 0 |
| THYMU2003891 | 1.703 | 0 | 0 | 0 | 3.998 | 0 | 0 |
| THYMU2003981 | 18.291 | 0 | 21.126 | 0 | 0 | 0 | 0 |
| THYMU2004152 | 0 | 0 | 0 | 0 | 0 | 7.337 | 0 |
| THYMU2004344 | 0 | 0 | 0 | 0 | 0 | 0 | 3.185 |
| THYMU2004410 | 0 | 0 | 0 | 0 | 0 | 0 | 4.776 |
| THYMU2004452 | 0 | 0 | 0 | 0 | 0 | 0 | 57.616 |
| THYMU2004733 | 0 | 0 | 0 | 33.006 | 0 | 0 | 0 |
| THYMU2004916 | 0 | 0 | 0 | 0 | 42.760 | 0 | 0 |
| THYMU2005546 | 0 | 0 | 0 | 3.223 | 0 | 2.021 | 0 |
| THYMU2006505 | 0 | 0 | 0 | 0 | 0 | 5.044 | 10.209 |
| THYMU2006813 | 0 | 0 | 0 | 0 | 0 | 8.770 | 3.945 |
| THYMU2007112 | 0 | 0 | 0 | 0 | 0 | 16.898 | 0 |
| THYMU2007415 | 0 | 0 | 0 | 0 | 0 | 8.941 | 0 |
| THYMU2007729 | 0 | 0 | 0 | 0 | 0 | 0 | 12.184 |
| THYMU2007802 | 0 | 0 | 0 | 0 | 0 | 0 | 74.935 |
| THYMU2008727 | 0 | 0 | 0 | 87.605 | 0 | 0 | 0 |
| THYMU2009658 | 0 | 0 | 4.723 | 0 | 0 | 0 | 0 |
| THYMU2011482 | 0 | 0 | 0 | 0 | 0 | 11.342 | 15.304 |
| THYMU2011736 | 0 | 0 | 0 | 0 | 0 | 0 | 74.935 |
| THYMU2012024 | 0 | 0 | 0 | 0 | 0 | 5.505 | 0 |
| THYMU2013136 | 0 | 0 | 0 | 0 | 0 | 0 | 4.078 |
| THYMU2013916 | 0 | 0 | 0 | 0 | 0 | 4.098 | 2.765 |
| THYMU2014167 | 0 | 0 | 0 | 0 | 0 | 10.152 | 0 |
| THYMU2015762 | 0 | 0 | 0 | 0 | 27.166 | 0 | 0 |
| THYMU2016968 | 86.295 | 0 | 0 | 0 | 0 | 0 | 0 |
| THYMU2017601 | 0 | 0 | 0 | 0 | 0 | 13.046 | 0 |
| THYMU2018028 | 0 | 0 | 0 | 0 | 0 | 0 | 74.935 |
| THYMU2027125 | 0 | 0 | 0 | 0 | 0 | 0 | 0.446 |
| THYMU2034374 | 0 | 0 | 0 | 0 | 0 | 23.239 | 0 |
| THYMU2036215 | 0 | 0 | 0 | 0 | 0 | 0 | 57.348 |
| THYMU3001077 | 0 | 0 | 0 | 0 | 0 | 0 | 74.935 |
| THYMU3013386 | 0 | 0 | 0 | 0 | 0 | 1.383 | 0 |
| THYMU3015759 | 0 | 0 | 0 | 0 | 0 | 2.080 | 0 |
| THYMU3032798 | 0 | 0 | 26.074 | 0 | 0 | 0 | 0 |
| THYMU3033626 | 0 | 0 | 0 | 0 | 0 | 10.384 | 0 |
| TKIDN1000192 | 0 | 0 | 0 | 0 | 0 | 10.206 | 0 |
| TKIDN2002738 | 0 | 0 | 0 | 0 | 0 | 0.775 | 0 |
| TKIDN2005487 | 0 | 0 | 9.687 | 0 | 0 | 0 | 0 |
| TKIDN2009641 | 0 | 0 | 0 | 0 | 0 | 21.363 | 0 |
| TKIDN2011289 | 0 | 0 | 0 | 0 | 0 | 11.044 | 14.902 |
| TKIDN2015423 | 7.432 | 0 | 0 | 0 | 0 | 0 | 4.761 |
| TLIVE2000979 | 0 | 0 | 0 | 0 | 0 | 0 | 26.611 |
| TLIVE2002046 | 0 | 0 | 0 | 0 | 0 | 3.353 | 4.524 |
| TRACH1000011 | 0 | 0 | 0 | 0 | 0 | 13.599 | 0 |
| TRACH1000030 | 0 | 0 | 0 | 0 | 0 | 2.549 | 3.440 |
| TRACH1000063 | 25.109 | 0 | 0 | 0 | 0 | 0 | 0 |
| TRACH1000181 | 0 | 0 | 0 | 0 | 0 | 0 | 11.810 |
| TRACH2000079 | 0 | 0 | 0 | 0 | 0 | 0 | 2.701 |
| TRACH2000248 | 0 | 0 | 0 | 0 | 0 | 9.022 | 3.478 |
| TRACH2000321 | 0 | 0 | 0 | 5.791 | 0 | 3.631 | 1.225 |
| TRACH2000359 | 0 | 0 | 0 | 0 | 0 | 0 | 7.434 |
| TRACH2000461 | 0 | 0 | 0 | 0 | 0 | 3.168 | 0 |
| TRACH2000559 | 0 | 0 | 0 | 0 | 0 | 7.195 | 0 |
| TRACH2000660 | 0 | 0 | 0 | 0 | 0 | 23.431 | 0 |
| TRACH2000780 | 10.204 | 0 | 0 | 0 | 0 | 0 | 0 |
| TRACH2000920 | 0 | 0 | 0 | 0 | 0 | 12.249 | 8.264 |
| TRACH2000926 | 0 | 0 | 0 | 0 | 0 | 0 | 6.955 |
| TRACH2000959 | 0 | 0 | 0 | 0 | 0 | 0.451 | 1.217 |
| TRACH2000972 | 0 | 0 | 7.991 | 0 | 0 | 1.095 | 0 |
| TRACH2001021 | 4.888 | 0 | 11.292 | 0 | 0 | 0 | 0 |
| TRACH2001109 | 0 | 0 | 0 | 0 | 0 | 2.707 | 1.043 |
| TRACH2001289 | 0 | 0 | 0 | 2.003 | 0 | 8.163 | 2.542 |
| TRACH2001314 | 0 | 0 | 0 | 0 | 0 | 3.377 | 0 |
| TRACH2001428 | 0 | 84.729 | 0 | 0 | 0 | 2.332 | 0 |
| TRACH2001432 | 0 | 0 | 0 | 0 | 0 | 4.110 | 2.773 |
| TRACH2001443 | 0 | 0 | 0 | 0 | 0 | 17.167 | 0 |
| TRACH2001463 | 0 | 0 | 0 | 0 | 0 | 11.313 | 7.633 |
| TRACH2001478 | 0 | 0 | 0 | 3.421 | 0 | 0 | 0 |
| TRACH2001523 | 0 | 0 | 22.925 | 0 | 0 | 3.141 | 0 |
| TRACH2001612 | 0 | 0 | 0 | 0 | 0 | 7.380 | 9.958 |
| TRACH2001810 | 0 | 0 | 0 | 0 | 0 | 7.173 | 5.808 |
| TRACH2002100 | 0 | 0 | 0 | 0 | 0 | 0 | 42.753 |
| TRACH2002537 | 0 | 0 | 0 | 5.465 | 0 | 5.140 | 0 |
| TRACH2002803 | 0 | 0 | 0 | 0 | 0 | 7.700 | 0 |
| TRACH2003272 | 0 | 0 | 0 | 0 | 0 | 6.845 | 9.236 |
| TRACH2003347 | 0 | 0 | 0 | 0 | 0 | 2.935 | 3.960 |
| TRACH2005811 | 0 | 0 | 0 | 0 | 0 | 1.948 | 7.887 |
| TRACH2007816 | 0 | 0 | 0 | 0 | 0 | 18.443 | 0 |
| TRACH2008081 | 0 | 85.762 | 0 | 0 | 0 | 0 | 0 |
| TRACH2008300 | 0 | 0 | 0 | 0 | 0 | 3.136 | 0 |
| TRACH2008472 | 0 | 0 | 0 | 9.247 | 0 | 0 | 0 |
| TRACH2011113 | 0 | 0 | 0 | 0 | 0 | 8.722 | 23.539 |
| TRACH2011894 | 0 | 0 | 0 | 0 | 0 | 32.856 | 0 |
| TRACH2013123 | 0 | 0 | 0 | 0 | 0 | 22.218 | 0 |
| TRACH2013552 | 0 | 0 | 0 | 0 | 0 | 0 | 25.898 |
| TRACH2014077 | 0 | 0 | 0 | 0 | 0 | 6.284 | 0 |
| TRACH2014124 | 12.051 | 0 | 0 | 0 | 0 | 3.814 | 5.146 |
| TRACH2015823 | 0 | 0 | 0 | 0 | 0 | 0 | 5.453 |
| TRACH2018446 | 0 | 0 | 0 | 0 | 0 | 15.452 | 0 |
| TRACH2023299 | 74.521 | 0 | 0 | 0 | 0 | 0 | 0 |
| TRACH2025535 | 6.778 | 0 | 0 | 0 | 0 | 3.218 | 0 |
| TRACH3002192 | 0 | 0 | 0 | 3.675 | 0 | 2.304 | 3.109 |
| TRACH3002752 | 0 | 0 | 0 | 0 | 0 | 9.436 | 0 |
| TRACH3004068 | 0 | 0 | 0 | 0 | 0 | 0 | 3.553 |
| TRACH3007625 | 0 | 0 | 0 | 6.928 | 0 | 0 | 0 |
| TUTER2000904 | 0 | 0 | 0 | 0 | 0 | 5.158 | 0 |
| UMVEN1000156 | 0 | 0 | 0 | 0 | 0 | 0 | 2.576 |
| UMVEN2000152 | 0 | 0 | 0 | 0 | 0 | 1.455 | 0 |
| UMVEN2000453 | 0 | 0 | 0 | 0 | 0 | 0 | 1.302 |
| UTERU1000106 | 0 | 0 | 0 | 0 | 0 | 10.646 | 14.365 |
| UTERU1000182 | 0 | 0 | 0 | 0 | 0 | 0 | 2.022 |
| UTERU2000095 | 0 | 0 | 0 | 0 | 0 | 1.529 | 0 |
| UTERU2000197 | 0 | 0 | 0 | 0 | 0 | 60.769 | 0 |
| UTERU2000300 | 0 | 0 | 0 | 0 | 0 | 9.112 | 0 |
| UTERU2000424 | 0 | 0 | 0 | 0 | 0 | 3.138 | 4.234 |
| UTERU2000542 | 0 | 0 | 0 | 0 | 0 | 0 | 22.508 |
| UTERU2001607 | 0 | 0 | 0 | 0 | 0 | 0 | 7.355 |
| UTERU2002294 | 0 | 0 | 0 | 0 | 0 | 0 | 13.576 |
| UTERU2002662 | 0 | 0 | 0 | 0 | 0 | 3.287 | 4.435 |
| UTERU2004461 | 0 | 0 | 0 | 0 | 0 | 0 | 67.639 |
| UTERU2004664 | 0 | 0 | 0 | 0 | 0 | 0 | 4.230 |
| UTERU2004807 | 0 | 0 | 0 | 0 | 0 | 0 | 2.504 |
| UTERU2004929 | 0 | 0 | 0 | 0 | 0 | 21.446 | 0 |
| UTERU2005621 | 0 | 0 | 0 | 0 | 0 | 0 | 5.619 |
| UTERU2006137 | 90.731 | 0 | 0 | 0 | 0 | 0 | 0 |
| UTERU2007253 | 0 | 0 | 0 | 0 | 0 | 38.074 | 0 |
| UTERU2008085 | 0 | 0 | 0 | 0 | 0 | 0.167 | 0.225 |
| UTERU2009510 | 0 | 0 | 0 | 0 | 0 | 27.767 | 0 |
| UTERU2009972 | 0 | 0 | 0 | 0 | 0 | 13.090 | 0 |
| UTERU2010417 | 0 | 0 | 0 | 1.858 | 0 | 1.165 | 0.786 |
| UTERU2011906 | 0 | 0 | 0 | 0 | 0 | 0 | 17.000 |
| UTERU2012230 | 0 | 0 | 0 | 0 | 0 | 0 | 4.387 |
| UTERU2012286 | 0 | 0 | 0 | 0 | 0 | 0 | 6.316 |
| UTERU2013078 | 0 | 0 | 0 | 0 | 0 | 0 | 34.190 |
| UTERU2013586 | 0 | 0 | 0 | 0 | 0 | 24.344 | 0 |
| UTERU2014167 | 0 | 61.187 | 0 | 0 | 0 | 1.684 | 0 |
| UTERU2014464 | 0 | 0 | 0 | 0 | 0 | 0 | 33.653 |
| UTERU2014548 | 0 | 0 | 0 | 0 | 0 | 60.769 | 0 |
| UTERU2014668 | 0 | 0 | 0 | 0 | 0 | 60.769 | 0 |
| UTERU2015653 | 0 | 0 | 0 | 3.438 | 0 | 0 | 0 |
| UTERU2016799 | 0 | 0 | 0 | 0 | 0 | 10.744 | 0 |
| UTERU2017613 | 0 | 0 | 0 | 0 | 0 | 0 | 51.268 |
| UTERU2019005 | 0 | 0 | 0 | 0 | 0 | 11.932 | 0 |
| UTERU2023175 | 0 | 0 | 0 | 0 | 0 | 8.252 | 0 |
| UTERU2027369 | 0 | 0 | 0 | 0 | 0 | 60.769 | 0 |
| UTERU3015011 | 0 | 0 | 0 | 0 | 0 | 0 | 17.593 |

Genes whose expression levels were negligibly low in each of the above-described libraries are shown with their clone names below.

3NB691000018, 3NB691000085, 3NB691000113, 3NB691000116, 3NB691000129, 3NB691000191, 3NB692000012, 3NB692000029, 3NB692000102, 3NB692000154, 3NB692000276, 3NB692000305, 3NB692000429, 3NB692000529, 3NB692000973, 3NB692001002, 3NB692001034, 3NB692001040, 3NB692001123, 3NB692001267, 3NB692001288, 3NB692001339, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001520, 3NB692001528, 3NB692001538, 3NB692001548, 3NB692001557, 3NB692001637, 3NB692001719, 3NB692001853, 3NB692001861, 3NB692002051, 3NB692002365, 3NB692002685, 3NB692002806, 3NB692003538, 3NB692004045, 3NB692004724, 3NB692005439, 3NB692008178, 3NB692008729, ACTVT2000380, ADIPS1000064, ADIPS2000069, ADIPS2000088, ADIPS2000245, ADRGL1000038, ADRGL1000067, ADRGL1000111, ADRGL1000144, ADRGL1000147, ADRGL1000160, ADRGL1000165, ADRGL1000182, ADRGL2000042, ADRGL2000056, ADRGL2000064, ADRGL2000074, ADRGL2000085, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO2000011, ASTRO2000095, ASTRO2000191, ASTRO2000278, ASTRO2000372, ASTRO2000381, ASTRO2000403, ASTRO2000417, ASTRO2000480, ASTRO2000482, ASTRO2000533, ASTRO2000653, ASTRO2000662, ASTRO2000725, ASTRO2000801, ASTRO2000914, ASTRO2001001, ASTRO2001008, ASTRO2001076, ASTRO2001088, ASTRO2001107, ASTRO2001122, ASTRO2001227, ASTRO2001249, ASTRO2001480, ASTRO2001806, ASTRO2002024, ASTRO2002035, ASTRO2002064, ASTRO2002459, ASTRO2002571, ASTRO2002632, ASTRO2002659, ASTRO2002693, ASTRO2002720, ASTRO2002733, ASTRO2002743, ASTRO2002842, ASTRO2003024, ASTRO2003316, ASTRO2003574, ASTRO2003581, ASTRO2003632, ASTRO2003740, ASTRO2003840, ASTRO2003925, ASTRO2003960, ASTRO2004051, ASTRO2004177, ASTRO2004217, ASTRO2004297, ASTRO2004584, ASTRO2004628, ASTRO2004751, ASTRO2004877, ASTRO2005008, ASTRO2005081, ASTRO2005096, ASTRO2005242, ASTRO2005273, ASTRO2005343, ASTRO2005360, ASTRO2005413, ASTRO2005423, ASTRO2005557, ASTRO2005575, ASTRO2005593, ASTRO2005726, ASTRO2005863, ASTRO2005896, ASTRO2005902, ASTRO2006356, ASTRO2006475, ASTRO2006732, ASTRO2006747, ASTRO2006952, ASTRO2007221, ASTRO2007377, ASTRO2007515, ASTRO2007578, ASTRO2007643, ASTRO2007666, ASTRO2007956, ASTRO2007962, ASTRO2008040, ASTRO2008216, ASTRO2008239, ASTRO2008409, ASTRO2008425, ASTRO2008508, ASTRO2008777, ASTRO2008895, ASTRO2008960, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014923, ASTRO2014961, ASTRO2015162, ASTRO2015185, ASTRO2015328, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018169, ASTRO2018373, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000123, BGGI11000193, BGGI11000285, BGGI12000022, BGGI12000067, BGGI12000161, BGGI12000533, BGGI12000544, BGGI12000616, BGGI12000684, BGGI12000693, BGGI12000839, BGGI12001002, BGGI12001241, BGGI12001247, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006415, BLADE2006607, BLADE2006826, BLADE2006830, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008479, BLADE2008539, BLADE2008809, BLADE2008995, BLADE2009452, BLADE2009479, BLADE2009593, BNGH41000104, BNGH41000118, BNGH41000137, BNGH41000153, BNGH41000169, BNGH41000177, BNGH41000190, BNGH41000198, BNGH41000216, BNGH41000235, BNGH42000130, BNGH42000132, BNGH42000360, BNGH42000405, BNGH42000532, BNGH42000675, BNGH42000791, BNGH42000815, BNGH42000994, BNGH42001247, BNGH42001406, BNGH42001576, BNGH42001724, BNGH42002168, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42006226, BNGH42006234, BNGH42007037, BNGH42007366, BNGH42007594, BNGH42007798, BNGH42008199, BNGH42008510, BNGH42008649, BNGH42008743, BNGH42008832, BNGH42008850, BNGH42008943, BNGH42009021, BRACE1000020, BRACE1000047, BRACE1000051, BRACE1000065, BRACE1000070, BRACE1000073, BRACE1000084, BRACE1000092, BRACE1000101, BRACE1000114, BRACE1000124, BRACE1000131, BRACE1000132, BRACE1000166, BRACE1000186, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000331, BRACE2000332, BRACE2000347, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000753, BRACE2000815, BRACE2000864, BRACE2000885, BRACE2000893, BRACE2000905, BRACE2000936, BRACE2000947, BRACE2000965, BRACE2000969, BRACE2000988, BRACE2001017, BRACE2001065, BRACE2001070, BRACE2001094, BRACE2001107, BRACE2001143, BRACE2001151, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001508, BRACE2001534, BRACE2001543, BRACE2001564, BRACE2001588, BRACE2001607, BRACE2001626, BRACE2001673, BRACE2001677, BRACE2001692, BRACE2001705, BRACE2001709, BRACE2001711, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001898, BRACE2001923, BRACE2001942, BRACE2001944, BRACE2001954, BRACE2001971, BRACE2001972, BRACE2001973, BRACE2002026, BRACE2002034, BRACE2002081, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002392, BRACE2002394, BRACE2002409, BRACE2002431, BRACE2002444, BRACE2002458, BRACE2002467, BRACE2002468, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002661, BRACE2002676, BRACE2002677, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002807, BRACE2002812, BRACE2002834, BRACE2002849, BRACE2002857, BRACE2002884, BRACE2002896, BRACE2002948, BRACE2003078, BRACE2003097, BRACE2003168, BRACE2003199, BRACE2003210, BRACE2003269, BRACE2003285, BRACE2003304, BRACE2003319, BRACE2003398, BRACE2003420, BRACE2003428, BRACE2003431, BRACE2003512, BRACE2003516, BRACE2003539, BRACE2003540, BRACE2003594, BRACE2003598, BRACE2003609, BRACE2003614, BRACE2003628, BRACE2003639, BRACE2003700, BRACE2003713, BRACE2003766, BRACE2003788, BRACE2003800, BRACE2003802, BRACE2003825, BRACE2003845, BRACE2003846, BRACE2003847, BRACE2003848, BRACE2003873, BRACE2003885, BRACE2003887, BRACE2003905, BRACE2003928, BRACE2003954, BRACE2003982, BRACE2003987, BRACE2005034, BRACE2005047, BRACE2005063, BRACE2005083, BRACE2005090, BRACE2005099, BRACE2005118, BRACE2005124, BRACE2005138, BRACE2005160, BRACE2005169, BRACE2005189, BRACE2005193, BRACE2005196, BRACE2005198, BRACE2005216, BRACE2005243, BRACE2005253, BRACE2005290, BRACE2005298, BRACE2005328, BRACE2005329, BRACE2005337, BRACE2005348, BRACE2005363, BRACE2005395, BRACE2005408, BRACE2005433, BRACE2005434, BRACE2005448, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005949, BRACE2005981, BRACE2005991, BRACE2006084, BRACE2006089, BRACE2006102, BRACE2006105, BRACE2006122, BRACE2006137, BRACE2006143, BRACE2006162, BRACE2006167, BRACE2006174, BRACE2006240, BRACE2006245, BRACE2006249, BRACE2006274, BRACE2006281, BRACE2006298, BRACE2006363, BRACE2006378, BRACE2006380, BRACE2006393, BRACE2006397, BRACE2006413, BRACE2006417, BRACE2006453, BRACE2006459, BRACE2006488, BRACE2006534, BRACE2006537, BRACE2006540, BRACE2006547, BRACE2006564, BRACE2006587, BRACE2006598, BRACE2006636, BRACE2006685, BRACE2006703, BRACE2006753, BRACE2006783, BRACE2006821, BRACE2006833, BRACE2006859, BRACE2006871, BRACE2006909, BRACE2006911, BRACE2006924, BRACE2006935, BRACE2006944, BRACE2006951, BRACE2006982, BRACE2006986, BRACE2007013, BRACE2007068, BRACE2007087, BRACE2007138, BRACE2007191, BRACE2007197, BRACE2007201, BRACE2007203, BRACE2007232, BRACE2007254, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007522, BRACE2007527, BRACE2007538, BRACE2007563, BRACE2007567, BRACE2007577, BRACE2007621, BRACE2007640, BRACE2007646, BRACE2007663, BRACE2007682, BRACE2007685, BRACE2007708, BRACE2007727, BRACE2007749, BRACE2007760, BRACE2007761, BRACE2007764, BRACE2007767, BRACE2007769, BRACE2007784, BRACE2007786, BRACE2007798, BRACE2007799, BRACE2007801, BRACE2007836, BRACE2007868, BRACE2007888, BRACE2007902, BRACE2007920, BRACE2007944, BRACE2008008, BRACE2008015, BRACE2008021, BRACE2008097, BRACE2008114, BRACE2008144, BRACE2008172, BRACE2008216, BRACE2008218, BRACE2008251, BRACE2008276, BRACE2008295, BRACE2008308, BRACE2008336, BRACE2008361, BRACE2008380, BRACE2008385, BRACE2008399, BRACE2008401, BRACE2008402, BRACE2008410, BRACE2008420, BRACE2008443, BRACE2008473, BRACE2008480, BRACE2008488, BRACE2008495, BRACE2008500, BRACE2008594, BRACE2008604, BRACE2008615, BRACE2008622, BRACE2008629, BRACE2008655, BRACE2008656, BRACE2008706, BRACE2008708, BRACE2008710, BRACE2008754, BRACE2008847, BRACE2008857, BRACE2008861, BRACE2008867, BRACE2008881, BRACE2008883, BRACE2008917, BRACE2008919, BRACE2008948, BRACE2008960, BRACE2008968, BRACE2008999, BRACE2009014, BRACE2009016, BRACE2009031, BRACE2009053, BRACE2009122, BRACE2009131, BRACE2009185, BRACE2009188, BRACE2009212, BRACE2009221, BRACE2009235, BRACE2009238, BRACE2009270, BRACE2009272, BRACE2009274, BRACE2009275, BRACE2009291, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009533, BRACE2009558, BRACE2009566, BRACE2009591, BRACE2009620, BRACE2009654, BRACE2009721, BRACE2009732, BRACE2009754, BRACE2009828, BRACE2009886, BRACE2009907, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010684, BRACE2010753, BRACE2010813, BRACE2010837, BRACE2010888, BRACE2010937, BRACE2010983, BRACE2011007, BRACE2011025, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012232, BRACE2012510, BRACE2012625, BRACE2012653, BRACE2012833, BRACE2012838, BRACE2012936, BRACE2012947, BRACE2013009, BRACE2013010, BRACE2013126, BRACE2013132, BRACE2013149, BRACE2013806, BRACE2013855, BRACE2013911, BRACE2014108, BRACE2014161, BRACE2014232, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015132, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017397, BRACE2017410, BRACE2017438, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021143, BRACE2021148, BRACE2021245, BRACE2021254, BRACE2021264, BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025710, BRACE2025992, BRACE2026131, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE30009.73, BRACE3001002, BRACE3001058, BRACE3001066, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004205, BRACE3004358, BRACE3004371, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY2000021, BRAMY2000025, BRAMY2000052, BRAMY2000076, BRAMY2000086, BRAMY2000151, BRAMY2000196, BRAMY2000231, BRAMY2000277, BRAMY2000388, BRAMY2000411, BRAMY2000508, BRAMY2000585, BRAMY2000653, BRAMY2000814, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2004031, BRAMY2004058, BRAMY2004183, BRAMY2004335, BRAMY2004351, BRAMY2004352, BRAMY2004387, BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007110, BRAMY2007185, BRAMY2007249, BRAMY2007255, BRAMY2007287, BRAMY2007308, BRAMY2007411, BRAMY2007458, BRAMY2007486, BRAMY2007546, BRAMY2007594, BRAMY2007610, BRAMY2007653, BRAMY2007753, BRAMY2007859, BRAMY2008333, BRAMY2008382, BRAMY2008563, BRAMY2008583, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009693, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012536, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016325, BRAMY2016327, BRAMY2016386, BRAMY2016706, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019600, BRAMY2019643, BRAMY2019710, BRAMY2019963, BRAMY2019985, BRAMY2020050, BRAMY2020058, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022525, BRAMY2022532, BRAMY2022723, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2024004, BRAMY2024073, BRAMY2024312, BRAMY2024374, BRAMY2024390, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026685, BRAMY2026713, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027739, BRAMY2027752, BRAMY2027782, BRAMY2028072, BRAMY2028183, BRAMY2028188, BRAMY2028269, BRAMY2028472, BRAMY2028491, BRAMY2028516, BRAMY2028565, BRAMY2028593, BRAMY2028682, BRAMY2028783, BRAMY2028796, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3003026, BRAMY3003109, BRAMY3003205, BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008088, BRAMY3008096, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010008, BRAMY3010264, BRAMY3010321, BRAMY3010411, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000007, BRAWH1000083, BRAWH1000094, BRAWH1000116, BRAWH1000130, BRAWH1000157, BRAWH1000164, BRAWH1000168, BRAWH1000174, BRAWH1000180, BRAWH1000369, BRAWH2000034, BRAWH2000048, BRAWH2000071, BRAWH2000093, BRAWH2000109, BRAWH2000131, BRAWH2000142, BRAWH2000177, BRAWH2000218, BRAWH2000232, BRAWH2000248, BRAWH2000256, BRAWH2000323, BRAWH2000353, BRAWH2000370, BRAWH2000409, BRAWH2000417, BRAWH2000443, BRAWH2000471, BRAWH2000476, BRAWH2000482, BRAWH2000488, BRAWH2000494, BRAWH2000500, BRAWH2000503, BRAWH2000554, BRAWH2000595, BRAWH2000633, BRAWH2000643, BRAWH2000651, BRAWH2000682, BRAWH2000697, BRAWH2000752, BRAWH2000859, BRAWH2000866, BRAWH2000892, BRAWH2000901, BRAWH2000926, BRAWH2000944, BRAWH2000984, BRAWH2001019, BRAWH2001052, BRAWH2001103, BRAWH2001129, BRAWH2001137, BRAWH2001159, BRAWH2001166, BRAWH2001186, BRAWH2001203, BRAWH2001238, BRAWH2001239, BRAWH2001255, BRAWH2001355, BRAWH2001378, BRAWH2001395, BRAWH2001406, BRAWH2001412, BRAWH2001418, BRAWH2001436, BRAWH2001438, BRAWH2001459, BRAWH2001461, BRAWH2001492, BRAWH2001503, BRAWH2001519, BRAWH2001530, BRAWH2001535, BRAWH2001589, BRAWH2001666, BRAWH2001671, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002963, BRAWH2002967, BRAWH2002976, BRAWH2003000, BRAWH2003135, BRAWH2003240, BRAWH2003307, BRAWH2003355, BRAWH2003366, BRAWH2003689, BRAWH2003693, BRAWH2003832, BRAWH2003948, BRAWH2003964, BRAWH2004044, BRAWH2004068, BRAWH2004078, BRAWH2004136, BRAWH2004217, BRAWH2004580, BRAWH2004731, BRAWH2004779, BRAWH2004842, BRAWH2004884, BRAWH2004923, BRAWH2005225, BRAWH2005315, BRAWH2005517, BRAWH2005524, BRAWH2005533, BRAWH2005578, BRAWH2005812, BRAWH2005974, BRAWH2005998, BRAWH2006044, BRAWH2006083, BRAWH2006207, BRAWH2006301, BRAWH2006395, BRAWH2006405, BRAWH2006526, BRAWH2006543, BRAWH2006622, BRAWH2006760, BRAWH2006960, BRAWH2006989, BRAWH2007069, BRAWH2007308, BRAWH2007406, BRAWH2007550, BRAWH2007570, BRAWH2007591, BRAWH2007658, BRAWH2007808, BRAWH2007825, BRAWH2007862, BRAWH2007890, BRAWH2008058, BRAWH2008128, BRAWH2008219, BRAWH2008292, BRAWH2008297, BRAWH2008366, BRAWH2008380, BRAWH2008540, BRAWH2008577, BRAWH2008790, BRAWH2008956, BRAWH2008993, BRAWH2009112, BRAWH2009227, BRAWH2009238, BRAWH2009261, BRAWH2009263, BRAWH2009298, BRAWH2009304, BRAWH2009307, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010354, BRAWH2010364, BRAWH2010536, BRAWH2010552, BRAWH2010584, BRAWH2010618, BRAWH2010619, BRAWH2010754, BRAWH2011066, BRAWH2011079, BRAWH2011096, BRAWH2011155, BRAWH2011166, BRAWH2011204, BRAWH2011286, BRAWH2011294, BRAWH2011400, BRAWH2011762, BRAWH2011795, BRAWH2011796, BRAWH2011823, BRAWH2011860, BRAWH2011920, BRAWH2011958, BRAWH2012054, BRAWH2012077, BRAWH2012162, BRAWH2012258, BRAWH2012326, BRAWH2012538, BRAWH2012540, BRAWH2012619, BRAWH2012698, BRAWH2012749, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014234, BRAWH2014379, BRAWH2014414, BRAWH2014473, BRAWH2014662, BRAWH2014717, BRAWH2014729, BRAWH2014876, BRAWH2014934, BRAWH2014954, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016679, BRAWH2016702, BRAWH2016785, BRAWH2016792, BRAWH2016800, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018729, BRAWH2018745, BRAWH2018875, BRAWH2019053, BRAWH2019055, BRAWH2019198, BRAWH3000078, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002574, BRAWH3002600, BRAWH3002819, BRAWH3003019, BRAWH3003136, BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016715, BRAWH3017260, BRAWH3017477, BRAWH3017537, BRAWH3017980, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021545, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000577, BRCAN2000620, BRCAN2000639, BRCAN2000763, BRCAN2000832, BRCAN2000923, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2010374, BRCAN2010376, BRCAN2010547, BRCAN2010581, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2025009, BRCAN2025093, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028040, BRCAN2028319, BRCAN2028338, BRCAN2028355, BRCAN2028378, BRCAN2028460, BRCAN2028545, BRCAN2028634, BRCAN2028702, BRCOC1000040, BRCOC1000087, BRCOC1000094, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003187, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019934, BRCOC2020122, BRCOC2020142, BRHIP1000058, BRHIP1000072, BRHIP1000129, BRHIP2000021, BRHIP2000079, BRHIP2000087, BRHIP2000163, BRHIP2000210, BRHIP2000239, BRHIP2000245, BRHIP2000359, BRHIP2000445, BRHIP2000506, BRHIP2000532, BRHIP2000621, BRHIP2000691, BRHIP2000819, BRHIP2000826, BRHIP2000920, BRHIP2001074, BRHIP2001686, BRHIP2001782, BRHIP2001802, BRHIP2001886, BRHIP2001927, BRHIP2002122, BRHIP2002143, BRHIP2002172, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007616, BRHIP2007690, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010309, BRHIP2010444, BRHIP2010487, BRHIP2010571, BRHIP2010593, BRHIP2010610, BRHIP2010723, BRHIP2010744, BRHIP2011120, BRHIP2011199, BRHIP2011491, BRHIP2011508, BRHIP2011576, BRHIP2011616, BRHIP2011783, BRHIP2011838, BRHIP2011891, BRHIP2011933, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387, BRHIP2014954, BRHIP2015153, BRHIP2015356, BRHIP2015360, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017714, BRHIP2017780, BRHIP2017945, BRHIP2018014, BRHIP2018369, BRHIP2018612, BRHIP2018635, BRHIP2018650, BRHIP2018719, BRHIP2018805, BRHIP2018998, BRHIP2019007, BRHIP2019047, BRHIP2019149, BRHIP2019177, BRHIP2019291, BRHIP2019494, BRHIP2019589, BRHIP2019641, BRHIP2019819, BRHIP2019884, BRHIP2019972, BRHIP2020182, BRHIP2020509, BRHIP2020622, BRHIP2020695, BRHIP2020743, BRHIP2020799, BRHIP2020842, BRHIP2020859, BRHIP2020930, BRHIP2021102, BRHIP2021289, BRHIP2021615, BRHIP2021744, BRHIP2021870, BRHIP2021929, BRHIP2022006, BRHIP2022054, BRHIP2022221, BRHIP2022228, BRHIP2022298, BRHIP2022326, BRHIP2022467, BRHIP2022708, BRHIP2023071, BRHIP2023229, BRHIP2023272, BRHIP2023309, BRHIP2023369, BRHIP2023438, BRHIP2023695, BRHIP2023735, BRHIP2023762, BRHIP2023860, BRHIP2023869, BRHIP2024046, BRHIP2024165, BRHIP2024201, BRHIP2024347, BRHIP2024398, BRHIP2024549, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026288, BRHIP2026346, BRHIP2026557, BRHIP2026723, BRHIP2026877, BRHIP2027017, BRHIP2027054, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000060, BRHIP3000070, BRHIP3000081, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002363, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006786, BRHIP3006950, BRHIP3007172, BRHIP3007183, BRHIP3007195, BRHIP3007223, BRHIP3007239, BRHIP3007291, BRHIP3007409, BRHIP3007424, BRHIP3007483, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3018278, BRHIP3018784, BRHIP3019643, BRHIP3019824, BRHIP3019880, BRHIP3019956, BRHIP3020155, BRHIP3020182, BRHIP3020733, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000059, BRSSN1000092, BRSSN1000153, BRSSN2000097, BRSSN2000175, BRSSN2000197, BRSSN2000244, BRSSN2000293, BRSSN2000295, BRSSN2000518, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2001869, BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009518, BRSSN2009627, BRSSN2009630, BRSSN2009702, BRSSN2010019, BRSSN2010110, BRSSN2010587, BRSSN2010596, BRSSN2010830, BRSSN2011262, BRSSN2011738, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014610, BRSSN2014636, BRSSN2014685, BRSSN2015199, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN201.8447, BRSSN2018581, BRSSN2018731, BRSSN2018771, BRSSN2018925, BRSTN1000097, BRSTN2000058, BRSTN2000070, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010107, BRSTN2010363, BRSTN2010416, BRSTN2010500, BRSTN2010569, BRSTN2010923, BRSTN2011211, BRSTN2011597, BRSTN2011688, BRSTN2011799, BRSTN2012284, BRSTN2012320, BRSTN2012380, BRSTN2012451, BRSTN2013171, BRSTN2013354, BRSTN2013931, BRSTN2014490, BRSTN2014633, BRSTN2014751, BRSTN2014770, BRSTN2015015, BRSTN2015889, BRSTN2015978, BRSTN2016066, BRSTN2016335, BRSTN2016678, BRSTN2016918, BRSTN2016992, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2007998, BRTHA2008316, BRTHA2008335, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003490, BRTHA3003704, BRTHA3003736, BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009291, BRTHA3010135, BRTHA3010366, BRTHA3010469, BRTHA3010540, BRTHA3010717, BRTHA3011149, BRTHA3011187, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3021971, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, CHONS2002829, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000009, CTONG1000010, CTONG1000020, CTONG1000022, CTONG1000040, CTONG1000056, CTONG1000062, CTONG1000086, CTONG1000088, CTONG1000093, CTONG1000097, CTONG1000113, CTONG1000137, CTONG1000165, CTONG1000175, CTONG1000180, CTONG1000181, CTONG1000220, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042, CTONG2000063, CTONG2000201, CTONG2000218, CTONG2000279, CTONG2000303, CTONG2000330, CTONG2000395, CTONG2000411, CTONG2000465, CTONG2000469, CTONG2000480, CTONG2000561, CTONG2000589, CTONG2000766, CTONG2000771, CTONG2000819, CTONG2000827, CTONG2000846, CTONG2000855, CTONG2000977, CTONG2001226, CTONG2001366, CTONG2001413, CTONG2001428, CTONG2001513, CTONG2001524, CTONG2001533, CTONG2001598, CTONG2001681, CTONG2001724, CTONG2001749, CTONG2001800, CTONG2001820, CTONG2001858, CTONG2001877, CTONG2001932, CTONG2001955, CTONG2002066, CTONG2002095, CTONG2002143, CTONG2002395, CTONG2002417, CTONG2002418, CTONG2002520, CTONG2002558, CTONG2002590, CTONG2002709, CTONG2002721, CTONG2002766, CTONG2002816, CTONG2002832, CTONG2002841, CTONG2002965, CTONG2002999, CTONG2003028, CTONG2003100, CTONG2003115, CTONG2003189, CTONG2003245, CTONG2003348, CTONG2003350, CTONG2003361, CTONG2003372, CTONG2003375, CTONG2003427, CTONG2003517, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004423, CTONG2004454, CTONG2004487, CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005278, CTONG2005374, CTONG2005399, CTONG2005468, CTONG2005567, CTONG2005585, CTONG2005615, CTONG2005635, CTONG2005775, CTONG2005795, CTONG2005890, CTONG2005904, CTONG2005913, CTONG2006004, CTONG2006129, CTONG2006223, CTONG2006273, CTONG2006310, CTONG2006377, CTONG2006393, CTONG2006449, CTONG2006515, CTONG2006524, CTONG2006568, CTONG2006611, CTONG2006620, CTONG2006666, CTONG2006691, CTONG2006836, CTONG2006942, CTONG2006964, CTONG2007007, CTONG2007009, CTONG2007072, CTONG2007078, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007613, CTONG2007623, CTONG2007681, CTONG2007779, CTONG2007898, CTONG2007959, CTONG2007970, CTONG2008014, CTONG2008184, CTONG2008262, CTONG2008269, CTONG2008321, CTONG2008343, CTONG2008402, CTONG2008405, CTONG2008466, CTONG2008562, CTONG2008577, CTONG2008595, CTONG2008621, CTONG2008689, CTONG2008720, CTONG2008721, CTONG2008773, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010348, CTONG2010408, CTONG2010508, CTONG2010623, CTONG2010652, CTONG2010803, CTONG2011188, CTONG2011429, CTONG2011474, CTONG2011676, CTONG2011716, CTONG2011765, CTONG2011779, CTONG2011815, CTONG2011822, CTONG2011825, CTONG2011920, CTONG2011985, CTONG2012029, CTONG2012077, CTONG2012101, CTONG2012123, CTONG2012158, CTONG2012230, CTONG2012422, CTONG2012425, CTONG2012447, CTONG2012453, CTONG2012473, CTONG2012476, CTONG2012533, CTONG2012554, CTONG2012564, CTONG2012607, CTONG2012661, CTONG2012724, CTONG2012738, CTONG2012745, CTONG2012758, CTONG2012843, CTONG2012847, CTONG2012879, CTONG2012901, CTONG2013128, CTONG2013149, CTONG2013156, CTONG2013178, CTONG2013222, CTONG2013284, CTONG2013339, CTONG2013352, CTONG2013393, CTONG2013511, CTONG2013630, CTONG2013803, CTONG2013934, CTONG2013986, CTONG2014032, CTONG2014058, CTONG2014119, CTONG2014191, CTONG2014206, CTONG2014234, CTONG2014264, CTONG2014280, CTONG2014369, CTONG2014491, CTONG2014630, CTONG2014635, CTONG2014646, CTONG2014705, CTONG2014946, CTONG2014954, CTONG2014959, CTONG2014966, CTONG2014995, CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016185, CTONG2016217, CTONG2016355, CTONG2016499, CTONG2016505, CTONG2016559, CTONG2016658, CTONG2016775, CTONG2016797, CTONG2016846, CTONG2016869, CTONG2016904, CTONG2016931, CTONG2016942, CTONG2016953, CTONG2017021, CTONG2017094, CTONG2017292, CTONG2017409, CTONG2017429, CTONG2017444, CTONG2017458, CTONG2017500, CTONG2017604, CTONG2017650, CTONG2017798, CTONG2017804, CTONG2017814, CTONG2017910, CTONG2017939, CTONG2017989, CTONG2017998, CTONG2018062, CTONG2018069, CTONG2018147, CTONG2018211, CTONG2018217, CTONG2018279, CTONG2018334, CTONG2018379, CTONG2018383, CTONG2018413, CTONG2018483, CTONG2018629, CTONG2018632, CTONG2018652, CTONG2018653, CTONG2018655, CTONG2018735, CTONG2018808, CTONG2018900, CTONG2018976, CTONG2019029, CTONG2019144, CTONG2019232, CTONG2019400, CTONG2019590, CTONG2019652, CTONG2019704, CTONG2019788, CTONG2019822, CTONG2019833, CTONG2020031, CTONG2020108, CTONG2020127, CTONG2020171, CTONG2020374, CTONG2020378, CTONG2020445, CTONG2020638, CTONG2020806, CTONG2020831, CTONG2020974, CTONG2021024, CTONG2021132, CTONG2021168, CTONG2021289, CTONG2022153, CTONG2022601, CTONG2023021, CTONG2023449, CTONG2023512, CTONG2024031, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028208, CTONG2028309, CTONG2028486, CTONG2028518, CTONG2028687, CTONG2028758, CTONG3000657, CTONG3000686, CTONG3000707, CTONG3000896, CTONG3001420, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002674, CTONG3002728, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009028, CTONG3009227, CTONG3009287, CTONG3009328, CTONG3009385, FEHRT2000325, FEHRT2000364, FEKID1000018, FELIV1000101, FELIV1000137, FELIV1000164, FELNG2000241, HCASM1000021, HCASM1000050, HCASM1000061, HCASM1000130, HCASM2000138, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000131, HCHON1000142, HCHON1000176, HCHON1000191, HCHON2000038, HCHON2000062, HCHON2000087, HCHON2000100, HCHON2000156, HCHON2000199, HCHON2000226, HCHON2000265, HCHON2000271, HCHON2000315, HCHON2000322, HCHON2000344, HCHON2000364, HCHON2000373, HCHON2000395, HCHON2000417, HCHON2000418, HCHON2000473, HCHON2000475, HCHON2000503, HCHON2000626, HCHON2000660, HCHON2000663, HCHON2000673, HCHON2000699, HCHON2000705, HCHON2000815, HCHON2000817, HCHON2000818, HCHON2000819, HCHON2000851, HCHON2000898, HCHON2000935, HCHON2000956, HCHON2000994, HCHON2001084, HCHON2001108, HCHON2001110, HCHON2001116, HCHON2001161, HCHON2001214, HCHON2001233, HCHON2001269, HCHON2001359, HCHON2001450, HCHON2001453, HCHON2001497, HCHON2001505, HCHON2001519, HCHON2001523, HCHON2001535, HCHON2001546, HCHON2001548, HCHON2001577, HCHON2001598, HCHON2001604, HCHON2001607, HCHON2001646, HCHON2001712, HCHON2001768, HCHON2001801, HCHON2001853, HCHON2002123, HCHON2002194, HCHON2002241, HCHON2002247, HCHON2002354, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005802, HCHON2005921, HCHON2005987, HCHON2006250, HCHON2006459, HCHON2006714, HCHON2006722, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HEART1000010, HEART1000074, HEART1000088, HEART1000099, HEART1000102, HEART1000118, HEART1000132, HEART1000139, HEART1000142, HEART1000151, HEART1000185, HEART2000024, HEART2000035, HEART2000099, HEART2000298, HEART2000306, HEART2000309, HEART2000411, HEART2000418, HEART2000444, HEART2000448, HEART2000492, HEART2000506, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006557, HEART2006787, HEART2006789, HEART2006909, HEART2007031, HEART2007443, HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC2000070, HHDPC2000102, HHDPC2000115, HHDPC2000258, HHDPC2000315, HHDPC2000456, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HLUNG1000017, HLUNG1000024, HLUNG1000030, HLUNG1000034, HLUNG1000037, HLUNG1000053, HLUNG1000064, HLUNG1000076, HLUNG1000084, HLUNG1000099, HLUNG1000104, HLUNG1000105, HLUNG1000110, HLUNG1000151, HLUNG1000169, HLUNG2000004, HLUNG2000014, HLUNG2000027, HLUNG2000068, HLUNG2000116, HLUNG2000125, HLUNG2000130, HLUNG2000142, HLUNG2000174, HLUNG2000176, HLUNG2000217, HLUNG2000255, HLUNG2000281, HLUNG2000315, HLUNG2000340, HLUNG2000362, HLUNG2000363, HLUNG2000431, HLUNG2000480, HLUNG2000489, HLUNG2000549, HLUNG2000557, HLUNG2000570, HLUNG2000751, HLUNG2000761, HLUNG2000777, HLUNG2000846, HLUNG2000870, HLUNG2000926, HLUNG2000955, HLUNG2000970, HLUNG2001013, HLUNG2001126, HLUNG2001144, HLUNG2001146, HLUNG2001214, HLUNG2001459, HLUNG2001507, HLUNG2001633, HLUNG2001677, HLUNG2001712, HLUNG2001814, HLUNG2001996, HLUNG2002006, HLUNG2002050, HLUNG2002085, HLUNG2002145, HLUNG2002295, HLUNG2002303, HLUNG2002405, HLUNG2002429, HLUNG2002465, HLUNG2002562, HLUNG2002648, HLUNG2002659, HLUNG2002707, HLUNG2002811, HLUNG2002916, HLUNG2002942, HLUNG2002949, HLUNG2002958, HLUNG2002982, HLUNG2003003, HLUNG2003042, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG20038.33, HLUNG2003872, HLUNG2003918, HLUNG2003991, HLUNG2004014, HLUNG2004037, HLUNG2004067, HLUNG2004154, HLUNG2004170, HLUNG2004217, HLUNG2004273, HLUNG2004388, HLUNG2004416, HLUNG2004521, HLUNG2004534, HLUNG2004684, HLUNG2004707, HLUNG2004775, HLUNG2005012, HLUNG2005030, HLUNG2005045, HLUNG2005076, HLUNG2005133, HLUNG2005203, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008139, HLUNG2008153, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008637, HLUNG2008833, HLUNG2008875, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000137, HSYRA1000148, HSYRA1000178, HSYRA2000044, HSYRA2000135, HSYRA2000137, HSYRA2000224, HSYRA2000237, HSYRA2000253, HSYRA2000255, HSYRA2000371, HSYRA2000424, HSYRA2000507, HSYRA2000510, HSYRA2000605, HSYRA2000629, HSYRA2000706, HSYRA2000760, HSYRA2000792, HSYRA2000828, HSYRA2000832, HSYRA2000925, HSYRA2000927, HSYRA2001003, HSYRA2001105, HSYRA2001142, HSYRA2001153, HSYRA2001232, HSYRA2001255, HSYRA2001332, HSYRA2001353, HSYRA2001420, HSYRA2001484, HSYRA2001552, HSYRA2001638, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000158, IMR321000161, IMR321000165, IMR321000207, IMR321000230, IMR321000266, IMR322000010, IMR322000072, IMR322000107, IMR322000152, IMR322000164, IMR322000178, IMR322000302, IMR322000374, IMR322000379, IMR322000383, IMR322000418, IMR322000518, IMR322000604, IMR322000609, IMR322000656, IMR322000672, IMR322000742, IMR322000775, IMR322000791, IMR322000811, IMR322000838, IMR322000859, IMR322000863, IMR322000919, IMR322000953, IMR322000984, IMR322001018, IMR322001049, IMR322001167, IMR322001185, IMR322001245, IMR322001317, IMR322001318, IMR322001322, IMR322001438, IMR322001452, IMR322001491, IMR322001534, IMR322001665, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322013053, IMR322013396, IMR322013731, IMR322013805, IMR322015523, IMR322016146, IMR322017049, IMR322018117, IMR322018192, JCMLC2000273, KIDNE1000008, KIDNE1000012, KIDNE1000015, KIDNE1000028, KIDNE1000036, KIDNE1000064, KIDNE1000104, KIDNE1000121, KIDNE1000143, KIDNE1000145, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000266, KIDNE2000287, KIDNE2000315, KIDNE2000317, KIDNE2000328, KIDNE2000330, KIDNE2000335, KIDNE2000349, KIDNE2000375, KIDNE2000383, KIDNE2000394, KIDNE2000422, KIDNE2000493, KIDNE2000497, KIDNE2000510, KIDNE2000513, KIDNE2000517, KIDNE2000519, KIDNE2000555, KIDNE2000624, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2000909, KIDNE2001117, KIDNE2001140, KIDNE2001269, KIDNE2001361, KIDNE2001373, KIDNE2001636, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002252, KIDNE2002262, KIDNE2002483, KIDNE2002725, KIDNE2002768, KIDNE2002795, KIDNE2002798, KIDNE2002839, KIDNE2002845, KIDNE2002882, KIDNE2002980, KIDNE2002991, KIDNE2003185, KIDNE2003188, KIDNE2003233, KIDNE2003305, KIDNE2003316, KIDNE2003335, KIDNE2003357, KIDNE2003377, KIDNE2003507, KIDNE2003752, KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004344, KIDNE2004394, KIDNE2004411, KIDNE2004519, KIDNE2004520, KIDNE2004531, KIDNE2004534, KIDNE2004562, KIDNE2004579, KIDNE2004681, KIDNE2004828, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006030, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006580, KIDNE2006652, KIDNE2006687, KIDNE2006733, KIDNE2006760, KIDNE2006775, KIDNE2006820, KIDNE2007005, KIDNE2007040, KIDNE2007077, KIDNE2007186, KIDNE2007222, KIDNE2007328, KIDNE2007356, KIDNE2007422, KIDNE2007569, KIDNE2007810, KIDNE2007811, KIDNE2007910, KIDNE2007944, KIDNE2007954, KIDNE2008022, KIDNE2008048, KIDNE2008069, KIDNE2008072, KIDNE2008116, KIDNE2008117, KIDNE2008218, KIDNE2008315, KIDNE2008362, KIDNE2008378, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012440, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539, KIDNE2016570, KIDNE2016918, KIDNE2017007, KIDNE2017040, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018462, KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000079, LIVER1000082, LIVER1000099, LIVER1000104, LIVER1000111, LIVER1000126, LIVER1000139, LIVER1000155, LIVER1000165, LIVER1000175, LIVER1000224, LIVER1000278, LIVER1000303, LIVER1000397, LIVER1000421, LIVER1000433, LIVER1000479, LIVER1000482, LIVER1000542, LIVER2000003, LIVER2000033, LIVER2000037, LIVER2000158, LIVER2000216, LIVER2000237, LIVER2000247, LIVER2000360, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001608, LIVER2001835, LIVER2002638, LIVER2002644, LIVER2002660, LIVER2002702, LIVER2003008, LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006251, LIVER2006410, LIVER2006469, LIVER2006644, LIVER2006764, LIVER2007415, LIVER2007548, LIVER2007568, LIVER2007721, LIVER2007783, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000096, MAMGL1000100, MAMGL1000132, MAMGL1000151, MAMGL1000173, MAMGL1000178, MAMGL1000182, MAMGL1000184, MESAN1000032, MESAN1000035, MESAN1000079, MESAN1000080, MESAN1000121, MESAN1000126, MESAN1000147, MESAN1000180, MESAN2000023, MESAN2000026, MESAN2000167, MESAN2000264, MESAN2000291, MESAN2000337, MESAN2000434, MESAN2000457, MESAN2000462, MESAN2000501, MESAN2000608, MESAN2000620, MESAN2000711, MESAN2000815, MESAN2000894, MESAN2000909, MESAN2001154, MESAN2001627, MESAN2001770, MESAN2001979, MESAN2001980, MESAN2002113, MESAN2002122, MESAN2002147, MESAN2002424, MESAN2002687, MESAN2002709, MESAN2002940, MESAN2002978, MESAN2003037, MESAN2003039, MESAN2003058, MESAN2003101, MESAN2003190, MESAN2003322, MESAN2003444, MESAN2003490, MESAN2003529, MESAN2003646, MESAN2003662, MESAN2003709, MESAN2003851, MESAN2004138, MESAN2005284, MESAN2005303, MESAN2005371, MESAN2005633, MESAN2005724, MESAN2005808, MESAN2005811, MESAN2005957, MESAN2005958, MESAN2006022, MESAN2006043, MESAN2006328, MESAN2006580, MESAN2006599, MESAN2006743, MESAN2006953, MESAN2007032, MESAN2007440, MESAN2007597, MESAN2008222, MESAN2008334, MESAN2008415, MESAN2008460, MESAN2008536, MESAN2008679, MESAN2008729, MESAN2008821, MESAN2009019, MESAN2009105, MESAN2009311, MESAN2009418, MESAN2009503, MESAN2009522, MESAN2009580, MESAN2010031, MESAN2010114, MESAN2010312, MESAN2010321, MESAN2010664, MESAN2010681, MESAN2011545, MESAN2011597, MESAN2011632, MESAN2011977, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016552, MESAN2016965, MESAN2017133, MESAN2017152, MESAN2017373, MESAN2017417, MESAN2018209, MESAN2018576, MESAN2018670, MESAN2018699, MESTC1000042, MESTC1000147, MESTC2000150, MESTC2000153, MESTC2000170, N1ESE2000698, NB9N41000047, NB9N41000121, NB9N41000135, NB9N41000142, NB9N41000146, NB9N41000340, NB9N42000122, NB9N42000196, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062, NHNPC2000606, NHNPC2000877, NHNPC2001108, NHNPC2001312, NHNPC2001816, NHNPC2002565, NHNPC2002749, NOVAR1000015, NOVAR1000091, NOVAR1000102, NOVAR2000038, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000004, NT2NE1000014, NT2NE1000023, NT2NE1000059, NT2NE1000063, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000174, NT2NE2000195, NT2NE2000222, NT2NE2000259, NT2NE2000284, NT2NE2000299, NT2NE2000356, NT2NE2000369, NT2NE2000374, NT2NE2000384, NT2NE2000390, NT2NE2000455, NT2NE2000488, NT2NE2000517, NT2NE2000536, NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000864, NT2NE2000913, NT2NE2000950, NT2NE2000951, NT2NE2000963, NT2NE2000980, NT2NE2001005, NT2NE2001021, NT2NE2001040, NT2NE2001049, NT2NE2001142, NT2NE2001156, NT2NE2001180, NT2NE2001181, NT2NE2001188, NT2NE2001191, NT2NE2001247, NT2NE2001252, NT2NE2001324, NT2NE2001326, NT2NE2001364, NT2NE2001372, NT2NE2001397, NT2NE2001403, NT2NE2001428, NT2NE2001432, NT2NE2001524, NT2NE2001598, NT2NE2001623, NT2NE2001626, NT2NE2001634, NT2NE2001648, NT2NE2001655, NT2NE2001660, NT2NE2001666, NT2NE2001677, NT2NE2001697, NT2NE2001698, NT2NE2001723, NT2NE2001725, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003252, NT2NE2003308, NT2NE2003309, NT2NE2003315, NT2NE2003408, NT2NE2003457, NT2NE2003540, NT2NE2003571, NT2NE2003705, NT2NE2003752, NT2NE2003887, NT2NE2003921, NT2NE2004378, NT2NE2004490, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005419, NT2NE2005441, NT2NE2005517, NT2NE2005537, NT2NE2005555, NT2NE2005588, NT2NE2005676, NT2NE2005720, NT2NE2005784, NT2NE2005804, NT2NE2005821, NT2NE2005890, NT2NE2005921, NT2NE2005968, NT2NE2006075, NT2NE2006217, NT2NE2006288, NT2NE2006382, NT2NE2006387, NT2NE2006427, NT2NE2006432, NT2NE2006478, NT2NE2006531, NT2NE2006659, NT2NE2006784, NT2NE2006813, NT2NE2006894, NT2NE2006942, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009426, NT2NE2009523, NT2NE2009608, NT2NE2009691, NT2NE2010105, NT2NE2010150, NT2NE2010400, NT2NE2010632, NT2NE2010633, NT2NE2010654, NT2NE2010688, NT2NE2010781, NT2NE2010854, NT2NE2011036, NT2NE2011107, NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018594, NT2NE2018739, NT2NE2018916, NT2NE2019056, NT2NE2019076, NT2NE2019099, NT2RI1000016, NT2RI1000027, NT2RI1000035, NT2RI1000164, NT2RI1000172, NT2RI2000004, NT2RI2000007, NT2RI2000107, NT2RI2000117, NT2RI2000128, NT2RI2000167, NT2RI2000173, NT2RI2000187, NT2RI2000263, NT2RI2000270, NT2RI2000276, NT2RI2000284, NT2RI2000313, NT2RI2000341, NT2RI2000421, NT2RI2000480, NT2RI2000575, NT2RI2000588, NT2RI2000597, NT2RI2000669, NT2RI2000671, NT2RI2000688, NT2RI2000689, NT2RI2000704, NT2RI2000738, NT2RI2000775, NT2RI2000822, NT2RI2000841, NT2RI2000865, NT2RI2000932, NT2RI2000974, NT2RI2000987, NT2RI2001010, NT2RI2001017, NT2RI2001030, NT2RI2001083, NT2RI2001167, NT2RI2001178, NT2RI2001230, NT2RI2001235, NT2RI2001244, NT2RI2001307, NT2RI2001409, NT2RI2001410, NT2RI2001425, NT2RI2001449, NT2RI2001474, NT2RI2001519, NT2RI2001595, NT2RI2001624, NT2RI2001726, NT2RI2001769, NT2RI2001782, NT2RI2001836, NT2RI2001846, NT2RI2001866, NT2RI2001910, NT2RI2002022, NT2RI2002041, NT2RI2002120, NT2RI2002153, NT2RI2002179, NT2RI2002243, NT2RI2002252, NT2RI2002260, NT2RI2002266, NT2RI2002270, NT2RI2002334, NT2RI2002359, NT2RI2002447, NT2RI2002481, NT2RI2002507, NT2RI2002517, NT2RI2002530, NT2RI2002549, NT2RI2002564, NT2RI2002585, NT2RI2002592, NT2RI2002602, NT2RI2002729, NT2RI2002802, NT2RI2002822, NT2RI2002852, NT2RI2002865, NT2RI2002923, NT2RI2002926, NT2RI2002970, NT2RI2003011, NT2RI2003067, NT2RI2003184, NT2RI2003205, NT2RI2003222, NT2RI2003338, NT2RI2003344, NT2RI2003360, NT2RI2003392, NT2RI2003399, NT2RI2003419, NT2RI2003481, NT2RI2003487, NT2RI2003655, NT2RI2003667, NT2RI2003678, NT2RI2003884, NT2RI2004059, NT2RI2004078, NT2RI2004079, NT2RI2004093, NT2RI2004136, NT2RI2004157, NT2RI2004188, NT2RI2004190, NT2RI2004284, NT2RI2004290, NT2RI2004304, NT2RI2004312, NT2RI2004381, NT2RI2004442, NT2RI2004468, NT2RI2004535, NT2RI2004606, NT2RI2004608, NT2RI2004618, NT2RI2004783, NT2RI2004818, NT2RI2004840, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005130, NT2RI2005166, NT2RI2005239, NT2RI2005246, NT2RI2005315, NT2RI2005358, NT2RI2005382, NT2RI2005473, NT2RI2005520, NT2RI2005621, NT2RI2005628, NT2RI2005647, NT2RI2005670, NT2RI2005710, NT2RI2005772, NT2RI2005814, NT2RI2005851, NT2RI2005966, NT2RI2006070, NT2RI2006071, NT2RI2006081, NT2RI2006126, NT2RI2006127, NT2RI2006257, NT2RI2006271, NT2RI2006294, NT2RI2006345, NT2RI2006403, NT2RI2006445, NT2RI2006487, NT2RI2006553, NT2RI2006565, NT2RI2006640, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006838, NT2RI2006853, NT2RI2006855, NT2RI2006973, NT2RI2007046, NT2RI2007048, NT2RI2007084, NT2RI2007096, NT2RI2007116, NT2RI2007148, NT2RI2007214, NT2RI2007303, NT2RI2007334, NT2RI2007386, NT2RI2007406, NT2RI2007439, NT2RI2007445, NT2RI2007469, NT2RI2007498, NT2RI2007507, NT2RI2007593, NT2RI2007729, NT2RI2007751, NT2RI2007754, NT2RI2007827, NT2RI2007853, NT2RI2007877, NT2RI2007879, NT2RI2007891, NT2RI2007956, NT2RI2007965, NT2RI2008007, NT2RI2008050, NT2RI2008141, NT2RI2008188, NT2RI2008204, NT2RI2008233, NT2RI2008455, NT2RI2008481, NT2RI2008502, NT2RI2008598, NT2RI2008622, NT2RI2008656, NT2RI2008714, NT2RI2008801, NT2RI2008812, NT2RI2008841, NT2RI2008952, NT2RI2009005, NT2RI2009066, NT2RI2009083, NT2RI2009101, NT2RI2009103, NT2RI2009144, NT2RI2009151, NT2RI2009215, NT2RI2009289, NT2RI2009301, NT2RI2009517, NT2RI2009583, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012659, NT2RI2012726, NT2RI2012990, NT2RI2013345, NT2RI2013357, NT2RI2013373, NT2RI2014247, NT2RI2014551, NT2RI2014683, NT2RI2014733, NT2RI2015239, NT2RI2016128, NT2RI2016134, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3001132, NT2RI3001263, NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006284, NT2RI3006340, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443, NT2RI3007543, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000005, NT2RP6000008, NT2RP6000032, NT2RP6000033, NT2RP6000039, NT2RP6000043, NT2RP6000059, NT2RP6000060, NT2RP6000077, NT2RP6000085, NT2RP6000086, NT2RP6000100, NT2RP6000119, NT2RP6000127, NT2RP6000130, NT2RP6000140, NT2RP6000150, NT2RP7000041, NT2RP7000069, NT2RP7000086, NT2RP7000109, NT2RP7000112, NT2RP7000259, NT2RP7000311, NT2RP7000359, NT2RP7000364, NT2RP7000380, NT2RP7000391, NT2RP7000579, NT2RP7000586, NT2RP7000600, NT2RP7000812, NT2RP7000926, NT2RP7001074, NT2RP7001080, NT2RP7001156, NT2RP7001306, NT2RP7001319, NT2RP7001335, NT2RP7001364, NT2RP7001475, NT2RP7001532, NT2RP7001602, NT2RP7001678, NT2RP7001856, NT2RP7001962, NT2RP7002064, NT2RP7002151, NT2RP7002163, NT2RP7002243, NT2RP7002282, NT2RP7002376, NT2RP7002449, NT2RP7002450, NT2RP7002478, NT2RP7002554, NT2RP7002594, NT2RP7002603, NT2RP7002619, NT2RP7002650, NT2RP7002779, NT2RP7002829, NT2RP7002841, NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003439, NT2RP7003629, NT2RP7003632, NT2RP7003647, NT2RP7003680, NT2RP7004027, NT2RP7004080, NT2RP7004114, NT2RP7004233, NT2RP7004260, NT2RP7004358, NT2RP7004396, NT2RP7004428, NT2RP7004541, NT2RP7004559, NT2RP7004641, NT2RP7004656, NT2RP7004687, NT2RP7004722, NT2RP7004766, NT2RP7004790, NT2RP7004911, NT2RP7004915, NT2RP7004961, NT2RP7004964, NT2RP7004975, NT2RP7005118, NT2RP7005205, NT2RP7005219, NT2RP7005323, NT2RP7005468, NT2RP7005493, NT2RP7005529, NT2RP7005629, NT2RP7005631, NT2RP7005669, NT2RP7005675, NT2RP7005750, NT2RP7005846, NT2RP7006033, NT2RP7006075, NT2RP7006141, NT2RP7006160, NT2RP7006162, NT2RP7006263, NT2RP7006296, NT2RP7006304, NT2RP7006374, NT2RP7006395, NT2RP7006408, NT2RP7006457, NT2RP7006490, NT2RP7006539, NT2RP7006547, NT2RP7006601, NT2RP7006621, NT2RP7006636, NT2RP7006701, NT2RP7006853, NT2RP7006886, NT2RP7006887, NT2RP7006980, NT2RP7006995, NT2RP7007154, NT2RP7007177, NT2RP7007221, NT2RP7007226, NT2RP7007252, NT2RP7007310, NT2RP7007359, NT2RP7007381, NT2RP7007406, NT2RP7007480, NT2RP7007504, NT2RP7007537, NT2RP7007580, NT2RP7007594, NT2RP7007610, NT2RP7007617, NT2RP7007643, NT2RP7007766, NT2RP7007925, NT2RP7007930, NT2RP7007975, NT2RP7008013, NT2RP7008133, NT2RP7008137, NT2RP7008142, NT2RP7008144, NT2RP7008161, NT2RP7008315, NT2RP7008360, NT2RP7008396, NT2RP7008406, NT2RP7008435, NT2RP7008454, NT2RP7008487, NT2RP7008543, NT2RP7008544, NT2RP7008550, NT2RP7008557, NT2RP7008623, NT2RP7008657, NT2RP7008720, NT2RP7008855, NT2RP7008863, NT2RP7009012, NT2RP7009019, NT2RP7009147, NT2RP7009149, NT2RP7009168, NT2RP7009215, NT2RP7009236, NT2RP7009253, NT2RP7009267, NT2RP7009322, NT2RP7009363, NT2RP7009370, NT2RP7009373, NT2RP7009397, NT2RP7009429, NT2RP7009439, NT2RP7009466, NT2RP7009502, NT2RP7009507, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7014910, NT2RP7015080, NT2RP7015431, NT2RP7015503, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8008057, NT2RP8009248, NTONG1000006, NTONG1000047, NTONG1000052, NTONG1000075, NTONG1000087, NTONG1000123, NTONG1000130, NTONG1000161, NTONG1000172, NTONG1000182, NTONG1000213, NTONG1000214, NTONG1000246, NTONG1000247, NTONG1000257, NTONG2000040, NTONG2000107, NTONG2000231, NTONG2000363, NTONG2000413, NTONG2000492, NTONG2000499, NTONG2000531, NTONG2000583, NTONG2000800, NTONG2000858, NTONG2000876, NTONG2000966, NTONG2001014, NTONG2001079, NTONG2001137, NTONG2001157, NTONG2001173, NTONG2001220, NTONG2001222, NTONG2001362, NTONG2001428, NTONG2001532, NTONG2001567, NTONG2001587, NTONG2001619, NTONG2001642, NTONG2001657, NTONG2001676, NTONG2002278, NTONG2002582, NTONG2002807, NTONG2002948, NTONG2002970, NTONG2002985, NTONG2003158, NTONG2003210, NTONG2003316, NTONG2003409, NTONG2003454, NTONG2003515, NTONG2003805, NTONG2003839, NTONG2003852, NTONG2003897, NTONG2003928, NTONG2004095, NTONG2004308, NTONG2004521, NTONG2004614, NTONG2004690, NTONG2004806, NTONG2004829, NTONG2004844, NTONG2004991, NTONG2005062, NTONG2005086, NTONG2005137, NTONG2005153, NTONG2005265, NTONG2005277, NTONG2005363, NTONG2005373, NTONG2005391, NTONG2005480, NTONG2005497, NTONG2005577, NTONG2005657, NTONG2005801, NTONG2005822, NTONG2006099, NTONG2006187, NTONG2006301, NTONG2006324, NTONG2006354, NTONG2006440, NTONG2006484, NTONG2006501, NTONG2006646, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008522, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030, OCBBF1000042, OCBBF1000054, OCBBF1000085, OCBBF1000086, OCBBF1000091, OCBBF1000118, OCBBF1000119, OCBBF1000122, OCBBF1000130, OCBBF1000138, OCBBF1000145, OCBBF1000175, OCBBF1000192, OCBBF1000254, OCBBF1000315, OCBBF2000231, OCBBF2000287, OCBBF2000452, OCBBF2000522, OCBBF2000677, OCBBF2000703, OCBBF2000712, OCBBF2000719, OCBBF2000824, OCBBF2000982, OCBBF2000998, OCBBF2001075, OCBBF2001101, OCBBF2001124, OCBBF2001140, OCBBF2001176, OCBBF2001186, OCBBF2001196, OCBBF2001307, OCBBF2001389, OCBBF2001408, OCBBF2001416, OCBBF2001473, OCBBF2001494, OCBBF2001527, OCBBF2001528, OCBBF2001586, OCBBF2001681, OCBBF2001706, OCBBF2001910, OCBBF2001931, OCBBF2001961, OCBBF2001983, OCBBF2002015, OCBBF2002083, OCBBF2002086, OCBBF2002124, OCBBF2002161, OCBBF2002357, OCBBF2002615, OCBBF2002805, OCBBF2002865, OCBBF2002920, OCBBF2002980, OCBBF2003028, OCBBF2003091, OCBBF2003327, OCBBF2003543, OCBBF2003593, OCBBF2003744, OCBBF2003819, OCBBF2004147, OCBBF2004168, OCBBF2004235, OCBBF2004259, OCBBF2004385, OCBBF2004418, OCBBF2004478, OCBBF2004482, OCBBF2004533, OCBBF2004567, OCBBF2004612, OCBBF2004647, OCBBF2004669, OCBBF2004826, OCBBF2004866, OCBBF2004883, OCBBF2004889, OCBBF2005066, OCBBF2005077, OCBBF2005161, OCBBF2005343, OCBBF2005349, OCBBF2005373, OCBBF2005420, OCBBF2005433, OCBBF2005476, OCBBF2005546, OCBBF2005843, OCBBF2005901, OCBBF2006005, OCBBF2006030, OCBBF2006058, OCBBF2006148, OCBBF2006151, OCBBF2006154, OCBBF2006214, OCBBF2006241, OCBBF2006388, OCBBF2006438, OCBBF2006567, OCBBF2006624, OCBBF2006639, OCBBF2006660, OCBBF2006972, OCBBF2006987, OCBBF2007039, OCBBF2007051, OCBBF2007068, OCBBF2007114, OCBBF2007121, OCBBF2007184, OCBBF2007194, OCBBF2007224, OCBBF2007238, OCBBF2007354, OCBBF2007414, OCBBF2007428, OCBBF2007478, OCBBF2007485, OCBBF2007520, OCBBF2007555, OCBBF2007610, OCBBF2007622, OCBBF2007756, OCBBF2007931, OCBBF2007946, OCBBF2008116, OCBBF2008138, OCBBF2008144, OCBBF2008283, OCBBF2008330, OCBBF2008366, OCBBF2008483, OCBBF2008511, OCBBF2008520, OCBBF2008569, OCBBF2008586, OCBBF2008640, OCBBF2008691, OCBBF2008724, OCBBF2008760, OCBBF2008768, OCBBF2008775, OCBBF2008790, OCBBF2008814, OCBBF2008822, OCBBF2009095, OCBBF2009115, OCBBF2009242, OCBBF2009352, OCBBF2009536, OCBBF2009571, OCBBF2009583, OCBBF2009603, OCBBF2009788, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010858, OCBBF2010863, OCBBF2010931, OCBBF2010945, OCBBF2010978, OCBBF2010989, OCBBF2011137, OCBBF2011160, OCBBF2011177, OCBBF2011228, OCBBF2011283, OCBBF2011311, OCBBF2011669, OCBBF2011685, OCBBF2011706, OCBBF2011722, OCBBF2011767, OCBBF2011855, OCBBF2011872, OCBBF2011897, OCBBF2011914, OCBBF2011960, OCBBF2011981, OCBBF2012001, OCBBF2012028, OCBBF2012039, OCBBF2012139, OCBBF2012262, OCBBF2012320, OCBBF2012436, OCBBF2012525, OCBBF2012704, OCBBF2012714, OCBBF2012755, OCBBF2012812, OCBBF2012881, OCBBF2012936, OCBBF2012990, OCBBF2013049, OCBBF2013091, OCBBF2013123, OCBBF2013127, OCBBF2013208, OCBBF2013285, OCBBF2013319, OCBBF2013324, OCBBF2013366, OCBBF2013507, OCBBF2013721, OCBBF2013883, OCBBF2014052, OCBBF2014089, OCBBF2014576, OCBBF2014707, OCBBF2014745, OCBBF2014828, OCBBF2015031, OCBBF2015081, OCBBF2015115, OCBBF2015232, OCBBF2015233, OCBBF2015285, OCBBF2015334, OCBBF2015335, OCBBF2015506, OCBBF2015645, OCBBF2015659, OCBBF2015797, OCBBF2015931, OCBBF2015980, OCBBF2016038, OCBBF2016467, OCBBF2016590, OCBBF2016591, OCBBF2016612, OCBBF2016689, OCBBF2016690, OCBBF2016729, OCBBF2016841, OCBBF2017055, OCBBF2017069, OCBBF2017306, OCBBF2017325, OCBBF2017489, OCBBF2017516, OCBBF2017665, OCBBF2017815, OCBBF2017882, OCBBF2017920, OCBBF2018014, OCBBF2018084, OCBBF2018167, OCBBF2018206, OCBBF2018229, OCBBF2018362, OCBBF2018380, OCBBF2018563, OCBBF2018581, OCBBF2018618, OCBBF2018663, OCBBF2018687, OCBBF2018827, OCBBF2018828, OCBBF2018934, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021323, OCBBF2021518, OCBBF2021788, OCBBF2021833, OCBBF2022351, OCBBF2022573, OCBBF2022574, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033413, OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000372, OCBBF3000380, OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004487, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000180, PEBLM2000030, PEBLM2000112, PEBLM2000126, PEBLM2000147, PEBLM2000170, PEBLM2000180, PEBLM2000213, PEBLM2000222, PEBLM2000248, PEBLM2000267, PEBLM2000270, PEBLM2000321, PEBLM2000326, PEBLM2000333, PEBLM2000338, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003935, PEBLM2004015, PEBLM2004216, PEBLM2004290, PEBLM2004452, PEBLM2004497, PEBLM2004666, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006709, PEBLM2006912, PEBLM2007112, PEBLM2007140, PEBLM2007188, PEBLM2007437, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007068, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000066, PLACE5000067, PLACE5000068, PLACE5000070, PLACE5000080, PLACE5000105, PLACE5000115, PLACE5000116, PLACE5000129, PLACE5000139, PLACE5000153, PLACE5000171, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000296, PLACE6000314, PLACE6000325, PLACE6000348, PLACE6000371, PLACE6000414, PLACE6000424, PLACE6000426, PLACE6000429, PLACE6000450, PLACE6000523, PLACE6000550, PLACE6000552, PLACE6000555, PLACE6000619, PLACE6000630, PLACE6000953, PLACE6001118, PLACE6001153, PLACE6001185, PLACE6001294, PLACE6001536, PLACE6001557, PLACE6001712, PLACE6001740, PLACE6001823, PLACE6001886, PLACE6001923, PLACE6001925, PLACE6001933, PLACE6002011, PLACE6002016, PLACE6002056, PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6002960, PLACE6003004, PLACE6003038, PLACE6003077, PLACE6003094, PLACE6003109, PLACE6003204, PLACE6003218, PLACE6003372, PLACE6003383, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004101, PLACE6004219, PLACE6004336, PLACE6004368, PLACE6004380, PLACE6004396, PLACE6004397, PLACE6004454, PLACE6004464, PLACE6004491, PLACE6004578, PLACE6004663, PLACE6004687, PLACE6004738, PLACE6004966, PLACE6004993, PLACE6005007, PLACE6005029, PLACE6005108, PLACE6005231, PLACE6005234, PLACE6005283, PLACE6005294, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006042, PLACE6006077, PLACE6006137, PLACE6006158, PLACE6006186, PLACE6006217, PLACE6006222, PLACE6006266, PLACE6006287, PLACE6006394, PLACE6006418, PLACE6006474, PLACE6006549, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008793, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010484, PLACE6010485, PLACE6010568, PLACE6010701, PLACE6010704, PLACE6010732, PLACE6010765, PLACE6010925, PLACE6010936, PLACE6010991, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012574, PLACE6012908, PLACE6012936, PLACE6012942, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016254, PLACE6016383, PLACE6016984, PLACE6017002, PLACE6017431, PLACE6017564, PLACE6017578, PLACE6017626, PLACE6017701, PLACE6017714, PLACE6017788, PLACE6017791, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6018938, PLACE6019195, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004103, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000110, PROST1000123, PROST1000136, PROST1000144, PROST1000145, PROST1000152, PROST1000167, PROST1000184, PROST1000217, PROST1000220, PROST1000226, PROST1000246, PROST1000262, PROST1000298, PROST1000322, PROST1000334, PROST1000343, PROST1000362, PROST1000377, PROST1000383, PROST1000412, PROST1000419, PROST1000451, PROST1000480, PROST1000485, PROST1000493, PROST1000512, PROST1000527, PROST1000528, PROST1000536, PROST1000559, PROST1000564, PROST1000575, PROST2000036, PROST2000053, PROST2000067, PROST2000105, PROST2000143, PROST2000162, PROST2000176, PROST2000203, PROST2000206, PROST2000212, PROST2000267, PROST2000273, PROST2000274, PROST2000277, PROST2000284, PROST2000325, PROST2000432, PROST2000452, PROST2000463, PROST2000470, PROST2000505, PROST2000567, PROST2000572, PROST2000717, PROST2000760, PROST2000761, PROST2000835, PROST2000961, PROST2001002, PROST2001116, PROST2001138, PROST2001283, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001899, PROST2001997, PROST2001998, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003232, PROST2003302, PROST2003303, PROST2003324, PROST2003340, PROST2003396, PROST2003428, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004332, PROST2004481, PROST2004526, PROST2004727, PROST2004739, PROST2004742, PROST2004744, PROST2004817, PROST2005067, PROST2005076, PROST2005093, PROST2005121, PROST2005131, PROST2005228, PROST2005272, PROST2005285, PROST2005426, PROST2005466, PROST2005604, PROST2005630, PROST2005683, PROST2005788, PROST2005880, PROST2005886, PROST2005904, PROST2005919, PROST2005943, PROST2006008, PROST2006020, PROST2006060, PROST2006196, PROST2006201, PROST2006260, PROST2006282, PROST2006343, PROST2006450, PROST2006491, PROST2006510, PROST2006579, PROST2006688, PROST2006737, PROST2006766, PROST2006782, PROST2006933, PROST2006988, PROST2007122, PROST2007200, PROST2007237, PROST2007248, PROST2007289, PROST2007317, PROST2007328, PROST2007382, PROST2007389, PROST2007416, PROST2007528, PROST2007818, PROST2007871, PROST2007950, PROST2007956, PROST2007974, PROST2007988, PROST2008079, PROST2008088, PROST2008243, PROST2008245, PROST2008447, PROST2008468, PROST2008472, PROST2008489, PROST2008516, PROST2008724, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010400, PROST2010545, PROST2010579, PROST2010606, PROST2010885, PROST2011012, PROST2011105, PROST2011297, PROST2011410, PROST2011439, PROST2011631, PROST2011660, PROST2012005, PROST2012016, PROST2012088, PROST2012140, PROST2012157, PROST2012190, PROST2012249, PROST2012353, PROST2012400, PROST2012504, PROST2012542, PROST2012550, PROST2012740, PROST2012745, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015137, PROST2015198, PROST2015243, PROST2015246, PROST2015251, PROST2015287, PROST2015457, PROST2015464, PROST2015537, PROST2015545, PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2016980, PROST2017098, PROST2017128, PROST2017203, PROST2017367, PROST2017413, PROST2017441, PROST2017578, PROST2017612, PROST2017617, PROST2017692, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2019164, PROST2019296, PROST2019398, PROST2019487, PROST2019781, PUAEN1000039, PUAEN1000065, PUAEN1000081, PUAEN1000085, PUAEN1000087, PUAEN1000121, PUAEN1000147, PUAEN1000161, PUAEN1000175, PUAEN1000275, PUAEN1000276, PUAEN1000288, PUAEN1000322, PUAEN2000080, PUAEN2000152, PUAEN2000175, PUAEN2000247, PUAEN2000394, PUAEN2000497, PUAEN2000594, PUAEN2000684, PUAEN2001260, PUAEN2001526, PUAEN2001586, PUAEN2001882, PUAEN2002473, PUAEN2002552, PUAEN2002568, PUAEN2002616, PUAEN2002758, PUAEN2003018, PUAEN2003079, PUAEN2003116, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007350, PUAEN2007785, PUAEN2007898, PUAEN2008123, PUAEN2008228, PUAEN2008314, PUAEN2008515, PUAEN2008815, PUAEN2008939, PUAEN2008980, PUAEN2008985, PUAEN2009174, PUAEN2009348, PUAEN2009534, PUAEN2009795, PUAEN2009852, PUAEN2010346, PUAEN2010824, RECTM1000141, RECTM2000220, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000047, SALGL1000065, SALGL1000107, SALGL1000171, SKMUS1000014, SKMUS1000030, SKMUS1000064, SKMUS1000067, SKMUS1000102, SKMUS1000104, SKMUS1000110, SKMUS1000118, SKMUS1000124, SKMUS1000129, SKMUS1000138, SKMUS1000176, SKMUS1000177, SKMUS2000061, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000365, SKMUS2000390, SKMUS2000416, SKMUS2000458, SKMUS2000467, SKMUS2000468, SKMUS2000515, SKMUS2000679, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000873, SKMUS2000894, SKMUS2000898, SKMUS2000933, SKMUS2000945, SKMUS2000954, SKMUS2000961, SKMUS2000973, SKMUS2000980, SKMUS2001008, SKMUS2001014, SKMUS2001129, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001412, SKMUS2001492, SKMUS2001501, SKMUS2001528, SKMUS2001543, SKMUS2001580, SKMUS2001585, SKMUS2001608, SKMUS2001631, SKMUS2001634, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000123, SKNMC1000124, SKNMC1000137, SKNMC1000159, SKNMC1000251, SKNMC1000264, SKNMC2000224, SKNMC2000256, SKNMC2000322, SKNMC2000374, SKNMC2000583, SKNMC2000593, SKNMC2000612, SKNMC2000622, SKNMC2000966, SKNMC2001113, SKNMC2001324, SKNMC2001503, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000086, SKNSH1000101, SKNSH1000174, SKNSH1000301, SKNSH1000308, SKNSH1000416, SKNSH2000051, SKNSH2000101, SKNSH2000151, SKNSH2000250, SKNSH2000319, SKNSH2000482, SKNSH2000516, SKNSH2000550, SKNSH2000971, SKNSH2001222, SKNSH2001875, SKNSH2001931, SKNSH2002054, SKNSH2002325, SKNSH2002768, SKNSH2002866, SKNSH2003064, SKNSH2003174, SKNSH2003422, SKNSH2003466, SKNSH2003483, SKNSH2003633, SKNSH2004039, SKNSH2005061, SKNSH2005120, SKNSH2005194, SKNSH2005240, SKNSH2005792, SKNSH2005816, SKNSH2005981, SKNSH2006304, SKNSH2006432, SKNSH2006822, SKNSH2007142, SKNSH2007149, SKNSH2007429, SKNSH2007739, SKNSH2008043, SKNSH2008969, SKNSH2009197, SKNSH2009435, SKNSH2009991, SKNSH2010015, SMINT1000012, SMINT1000016, SMINT1000039, SMINT1000042, SMINT1000048, SMINT1000051, SMINT1000054, SMINT1000057, SMINT1000071, SMINT1000075, SMINT1000078, SMINT1000091, SMINT1000100, SMINT1000103, SMINT1000118, SMINT1000131, SMINT1000137, SMINT1000192, SMINT2000018, SMINT2000032, SMINT2000040, SMINT2000060, SMINT2000084, SMINT2000145, SMINT2000159, SMINT2000176, SMINT2000185, SMINT2000227, SMINT2000229, SMINT2000232, SMINT2000239, SMINT2000267, SMINT2000277, SMINT2000400, SMINT2000454, SMINT2000518, SMINT2000529, SMINT2000530, SMINT2000537, SMINT2000538, SMINT2000541, SMINT2000545, SMINT2000558, SMINT2000562, SMINT2000568, SMINT2000609, SMINT2000659, SMINT2000747, SMINT2000811, SMINT2000824, SMINT2000933, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001818, SMINT2001950, SMINT2002126, SMINT2002202, SMINT2002210, SMINT2002224, SMINT2002281, SMINT2002311, SMINT2002314, SMINT2002328, SMINT2002414, SMINT2002457, SMINT2002743, SMINT2002778, SMINT2002880, SMINT2002882, SMINT2002899, SMINT2002974, SMINT2002976, SMINT2003003, SMINT2003128, SMINT2003150, SMINT2003317, SMINT2003386, SMINT2003423, SMINT2003551, SMINT2003569, SMINT2003641, SMINT2003644, SMINT2003866, SMINT2003905, SMINT2004023, SMINT2004037, SMINT2004047, SMINT2004086, SMINT2004144, SMINT2004280, SMINT2004299, SMINT2004339, SMINT2004350, SMINT2004414, SMINT2004473, SMINT2004547, SMINT2004583, SMINT2004589, SMINT2004781, SMINT2004891, SMINT2004909, SMINT2004992, SMINT2005075, SMINT2005161, SMINT2005174, SMINT2005213, SMINT2005330, SMINT2005387, SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006801, SMINT2007062, SMINT2007101, SMINT2007140, SMINT2007187, SMINT2007219, SMINT2007294, SMINT2007365, SMINT2007391, SMINT2007433, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2009119, SMINT2009121, SMINT2009212, SMINT2009216, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010144, SMINT2010200, SMINT2010278, SMINT2010324, SMINT2010326, SMINT2010369, SMINT2010500, SMINT2010533, SMINT2010629, SMINT2010639, SMINT2010672, SMINT2010753, SMINT2010853, SMINT2010897, SMINT2010959, SMINT2010997, SMINT2011033, SMINT2011066, SMINT2011273, SMINT2011311, SMINT2011406, SMINT2011509, SMINT2011567, SMINT2011656, SMINT2011888, SMINT2011958, SMINT2012040, SMINT2012122, SMINT2012179, SMINT2012195, SMINT2012285, SMINT2012291, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2019017, SMINT2019105, SMINT2019142, SMINT2019153, SMINT2019200, SPLEN1000013, SPLEN1000041, SPLEN1000056, SPLEN1000087, SPLEN1000091, SPLEN1000134, SPLEN1000139, SPLEN1000143, SPLEN1000144, SPLEN1000145, SPLEN1000156, SPLEN1000163, SPLEN1000165, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000210, SPLEN2000222, SPLEN2000242, SPLEN2000243, SPLEN2000247, SPLEN2000258, SPLEN2000270, SPLEN2000307, SPLEN2000310, SPLEN2000341, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695, SPLEN2000839, SPLEN2000874, SPLEN2001135, SPLEN2001141, SPLEN2001157, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001626, SPLEN2001650, SPLEN2001710, SPLEN2001761, SPLEN2001781, SPLEN2001881, SPLEN2001912, SPLEN2001945, SPLEN2002053, SPLEN2002133, SPLEN2002147, SPLEN2002166, SPLEN2002175, SPLEN2002223, SPLEN2002272, SPLEN2002314, SPLEN2002343, SPLEN2002354, SPLEN2002385, SPLEN2002419, SPLEN2002451, SPLEN2002462, SPLEN2002463, SPLEN2002477, SPLEN2002493, SPLEN2002662, SPLEN2002695, SPLEN2002707, SPLEN2002744, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004124, SPLEN2004148, SPLEN2004181, SPLEN2004220, SPLEN2004343, SPLEN2004346, SPLEN2004368, SPLEN2004611, SPLEN2004662, SPLEN2004844, SPLEN2004880, SPLEN2004984, SPLEN2005227, SPLEN2005272, SPLEN2005416, SPLEN2005429, SPLEN2005450, SPLEN2005474, SPLEN2005560, SPLEN2005727, SPLEN2005783, SPLEN2005790, SPLEN2005806, SPLEN2005818, SPLEN2005838, SPLEN2005927, SPLEN2005939, SPLEN2006268, SPLEN2006283, SPLEN2006325, SPLEN2006389, SPLEN2006425, SPLEN2006671, SPLEN2006701, SPLEN2006785, SPLEN2006875, SPLEN2007182, SPLEN2007314, SPLEN2007326, SPLEN2007350, SPLEN2007388, SPLEN2007619, SPLEN2007653, SPLEN2007689, SPLEN2007695, SPLEN2007739, SPLEN2007750, SPLEN2007794, SPLEN2007879, SPLEN2007926, SPLEN2008007, SPLEN2008135, SPLEN2008164, SPLEN2008317, SPLEN2008496, SPLEN2008591, SPLEN2008786, SPLEN2009088, SPLEN2009163, SPLEN2009315, SPLEN2009340, SPLEN2009512, SPLEN2009513, SPLEN2009541, SPLEN2009548, SPLEN2009555, SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011678, SPLEN2011737, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012523, SPLEN2012524, SPLEN2012525, SPLEN2012606, SPLEN2012611, SPLEN2012619, SPLEN2012624, SPLEN2012725, SPLEN2012743, SPLEN2012756, SPLEN2012800, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013857, SPLEN2013860, SPLEN2013910, SPLEN2013923, SPLEN2014041, SPLEN2014063, SPLEN2014073, SPLEN2014119, SPLEN2014136, SPLEN2014199, SPLEN2014210, SPLEN2014293, SPLEN2014318, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014911, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016241, SPLEN2016250, SPLEN2016304, SPLEN2016356, SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016773, SPLEN2016781, SPLEN2016840, SPLEN2016863, SPLEN2016922, SPLEN2016932, SPLEN2016973, SPLEN2017043, SPLEN2017104, SPLEN2017121, SPLEN2017147, SPLEN2017187, SPLEN2017189, SPLEN2017212, SPLEN2017351, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017918, SPLEN2017999, SPLEN2018098, SPLEN2018112, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019257, SPLEN2019311, SPLEN2019323, SPLEN2019349, SPLEN2019375, SPLEN2019379, SPLEN2019446, SPLEN2019471, SPLEN2019480, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028417, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031547, SPLEN2031674, SPLEN2031724, SPLEN2031780, SPLEN2032036, SPLEN2032112, SPLEN2032154, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042535, SPLEN2042598, SPLEN2042714, SPLEN2042920, STOMA1000013, STOMA1000047, STOMA1000052, STOMA1000074, STOMA1000091, STOMA1000117, STOMA1000133, STOMA1000175, STOMA1000186, STOMA1000190, STOMA1000191, STOMA2000032, STOMA2000072, STOMA2000088, STOMA2000104, STOMA2000121, STOMA2000148, STOMA2000168, STOMA2000183, STOMA2000188, STOMA2000257, STOMA2000279, STOMA2000289, STOMA2000383, STOMA2000386, STOMA2000396, STOMA2000478, STOMA2000482, STOMA2000529, STOMA2000539, STOMA2000567, STOMA2000640, STOMA2000678, STOMA2000686, STOMA2000888, STOMA2001025, STOMA2001389, STOMA2002052, STOMA2002141, STOMA2002166, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049, SYNOV1000118, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001212, SYNOV2001239, SYNOV2001251, SYNOV2001300, SYNOV2001356, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001153, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007711, SYNOV4008245, SYNOV4008336, SYNOV4009139, SYNOV4009298, SYNOV4009575, T1ESE2000116, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000163, TESTI1000168, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000117, TESTI2000120, TESTI2000147, TESTI2000154, TESTI2000172, TESTI2000177, TESTI2000179, TESTI2000207, TESTI2000208, TESTI2000221, TESTI2000253, TESTI2000272, TESTI2000278, TESTI2000349, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082, TESTI2001120, TESTI2001134, TESTI2001153, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001829, TESTI2001852, TESTI2001862, TESTI2001869, TESTI2001879, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002365, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002707, TESTI2002724, TESTI2002729, TESTI2002752, TESTI2002753, TESTI2002789, TESTI2002802, TESTI2002806, TESTI2002866, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003418, TESTI2003419, TESTI2003432, TESTI2003475, TESTI2003476, TESTI2003498, TESTI2003512, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004080, TESTI2004085, TESTI2004089, TESTI2004111, TESTI2004119, TESTI2004122, TESTI2004163, TESTI2004169, TESTI2004207, TESTI2004215, TESTI2004227, TESTI2004229, TESTI2004243, TESTI2004255, TESTI2004287, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004737, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994, TESTI2004999, TESTI2005017, TESTI2005025, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415, TESTI2005470, TESTI2005492, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005743, TESTI2005759, TESTI2005767, TESTI2005775, TESTI2005788, TESTI2005827, TESTI2005835, TESTI2005860, TESTI2005892, TESTI2005911, TESTI2005933, TESTI2005937, TESTI2005948, TESTI2005979, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006111, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006457, TESTI2006460, TESTI2006465, TESTI2006483, TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006774, TESTI2006853, TESTI2006866, TESTI2006872, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007311, TESTI2007320, TESTI2007346, TESTI2007358, TESTI2007402, TESTI2007405, TESTI2007422, TESTI2007452, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009412, TESTI2009423, TESTI2009447, TESTI2009462, TESTI2009474, TESTI2009477, TESTI2009497, TESTI2009502, TESTI2009511, TESTI2009544, TESTI2009545, TESTI2009551, TESTI2009577, TESTI2009585, TESTI2009588, TESTI2009683, TESTI2009727, TESTI2009739, TESTI2009812, TESTI2009835, TESTI2009853, TESTI2009935, TESTI2009977, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012155, TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012778, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013257, TESTI2013268, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014577, TESTI2014578, TESTI2014716, TESTI2014800, TESTI2014838, TESTI2014843, TESTI2014979, TESTI2014988, TESTI2015042, TESTI2015092, TESTI2015105, TESTI2015180, TESTI2015246, TESTI2015249, TESTI2015327, TESTI2015331, TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2017954, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018867, TESTI2018941, TESTI2018957, TESTI2018974, TESTI2018975, TESTI2018997, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021138, TESTI2021297, TESTI2021358, TESTI2021425, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033441, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002141, TESTI4002195, TESTI4002202, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003733, TESTI4003796, TESTI4003944, TESTI4004003, TESTI4004031, TESTI4004200, TESTI4004210, TESTI4004539, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013474, TESTI4013602, TESTI4013667, TESTI4013675, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000025, THYMU1000029, THYMU1000032, THYMU1000033, THYMU1000060, THYMU1000074, THYMU1000083, THYMU1000098, THYMU1000105, THYMU1000109, THYMU1000136, THYMU1000142, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000329, THYMU1000331, THYMU1000336, THYMU1000366, THYMU1000382, THYMU1000393, THYMU1000394, THYMU1000399, THYMU1000426, THYMU1000428, THYMU1000440, THYMU1000459, THYMU1000473, THYMU1000491, THYMU1000496, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000440, THYMU2000489, THYMU2000492, THYMU2000519, THYMU2000570, THYMU2000602, THYMU2000660, THYMU2000671, THYMU2000702, THYMU2000767, THYMU2000775, THYMU2000800, THYMU2000879, THYMU2000932, THYMU2000950, THYMU2000971, THYMU2000982, THYMU2000990, THYMU2001007, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001839, THYMU2001868, THYMU2001898, THYMU2001900, THYMU2001916, THYMU2001989, THYMU2002023, THYMU2002037, THYMU2002080, THYMU2002109, THYMU2002147, THYMU2002154, THYMU2002157, THYMU2002177, THYMU2002203, THYMU2002229, THYMU2002242, THYMU2002338, THYMU2002356, THYMU2002376, THYMU2002384, THYMU2002490, THYMU2002548, THYMU2002695, THYMU2002745, THYMU2002789, THYMU2002792, THYMU2002815, THYMU2002852, THYMU2002910, THYMU2002950, THYMU2003046, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003470, THYMU2003479, THYMU2003499, THYMU2003632, THYMU2003650, THYMU2003700, THYMU2003760, THYMU2003803, THYMU2003855, THYMU2003907, THYMU2003932, THYMU2004003, THYMU2004008, THYMU2004042, THYMU2004108, THYMU2004111, THYMU2004123, THYMU2004139, THYMU2004169, THYMU2004284, THYMU2004320, THYMU2004336, THYMU2004356, THYMU2004508, THYMU2004512, THYMU2004635, THYMU2004639, THYMU2004668, THYMU2004677, THYMU2004688, THYMU2004693, THYMU2004835, THYMU2004843, THYMU2004906, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006913, THYMU2006946, THYMU2006965, THYMU2007025, THYMU2007036, THYMU2007060, THYMU2007065, THYMU2007095, THYMU2007146, THYMU2007179, THYMU2007258, THYMU2007307, THYMU2007308, THYMU2007339, THYMU2007350, THYMU2007384, THYMU2007467, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007824, THYMU2007854, THYMU2007886, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452, THYMU2008643, THYMU2008686, THYMU2008691, THYMU2008714, THYMU2008725, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010448, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011538, THYMU2011548, THYMU2011551, THYMU2011564, THYMU2011573, THYMU2011585, THYMU2011621, THYMU2011666, THYMU2011785, THYMU2011806, THYMU2011852, THYMU2011875, THYMU2011881, THYMU2011937, THYMU2011939, THYMU2012002, THYMU2012073, THYMU2012104, THYMU2012113, THYMU2012273, THYMU2012401, THYMU2012492, THYMU2012507, THYMU2012565, THYMU2012574, THYMU2012625, THYMU2012631, THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017675, THYMU2017707, THYMU2017831, THYMU2017844, THYMU2017878, THYMU2017948, THYMU2018071, THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2035735, THYMU2036058, THYMU2036105, THYMU2036207, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032867, THYMU3033402, THYMU3033630, THYMU3033649, THYMU3033754, THYMU3033759, THYMU3034078, THYMU3034099, THYMU3034453, THYMU3034616, THYMU3034671, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000066, TKIDN1000164, TKIDN1000171, TKIDN2000127, TKIDN2000240, TKIDN2000246, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2003044, TKIDN2003059, TKIDN2003062, TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2001327, TLIVE2001616, TLIVE2001684, TLIVE2001828, TLIVE2001927, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000018, TRACH1000038, TRACH1000057, TRACH1000074, TRACH1000100, TRACH1000106, TRACH1000125, TRACH1000140, TRACH1000144, TRACH1000193, TRACH1000205, TRACH1000212, TRACH1000240, TRACH2000002, TRACH2000015, TRACH2000024, TRACH2000032, TRACH2000045, TRACH2000047, TRACH2000081, TRACH2000083, TRACH2000148, TRACH2000185, TRACH2000196, TRACH2000235, TRACH2000237, TRACH2000243, TRACH2000250, TRACH2000259, TRACH2000272, TRACH2000282, TRACH2000287, TRACH2000289, TRACH2000306, TRACH2000312, TRACH2000336, TRACH2000393, TRACH2000411, TRACH2000420, TRACH2000472, TRACH2000496, TRACH2000497, TRACH2000502, TRACH2000522, TRACH2000540, TRACH2000665, TRACH2000675, TRACH2000677, TRACH2000702, TRACH2000703, TRACH2000767, TRACH2000775, TRACH2000807, TRACH2000862, TRACH2000873, TRACH2000894, TRACH2000898, TRACH2000923, TRACH2000944, TRACH2000981, TRACH2000986, TRACH2001037, TRACH2001060, TRACH2001085, TRACH2001101, TRACH2001121, TRACH2001147, TRACH2001154, TRACH2001188, TRACH2001192, TRACH2001249, TRACH2001275, TRACH2001335, TRACH2001339, TRACH2001341, TRACH2001347, TRACH2001395, TRACH2001400, TRACH2001403, TRACH2001416, TRACH2001474, TRACH2001525, TRACH2001532, TRACH2001549, TRACH2001592, TRACH2001596, TRACH2001607, TRACH2001621, TRACH2001646, TRACH2001651, TRACH2001684, TRACH2001898, TRACH2001933, TRACH2001968, TRACH2001996, TRACH2002043, TRACH2002054, TRACH2002092, TRACH2002138, TRACH2002333, TRACH2002664, TRACH2002784, TRACH2002840, TRACH2002847, TRACH2002853, TRACH2002862, TRACH2002954, TRACH2002960, TRACH2002988, TRACH2003070, TRACH2003323, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005800, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007399, TRACH2007483, TRACH2007542, TRACH2007577, TRACH2007674, TRACH2007676, TRACH2007688, TRACH2007733, TRACH2007754, TRACH2007894, TRACH2007969, TRACH2007988, TRACH2008112, TRACH2008113, TRACH2008127, TRACH2008130, TRACH2008278, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011290, TRACH2011293, TRACH2011302, TRACH2011500, TRACH2011574, TRACH2011956, TRACH2012000, TRACH2012138, TRACH2012242, TRACH2012298, TRACH2012370, TRACH2012376, TRACH2012387, TRACH2012497, TRACH2012536, TRACH2012562, TRACH2012742, TRACH2012811, TRACH2012823, TRACH2012918, TRACH2013081, TRACH2013265, TRACH2013369, TRACH2013450, TRACH2013495, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084, TRACH2018262, TRACH2018273, TRACH2018278, TRACH2018317, TRACH2018345, TRACH2018380, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002866, TRACH3002871, TRACH3002876, TRACH3002890, TRACH3003009, TRACH3003037, TRACH3003357, TRACH3003379, TRACH3003547, TRACH3003586, TRACH3003683, TRACH3003780, TRACH3003805, TRACH3003832, TRACH3003872, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007627, TRACH3007689, TRACH3007766, TRACH3007866, TRACH3007995, TRACH3008042, TRACH3008061, TRACH3008093, TRACH3008201, TRACH3008508, TRACH3008535, TRACH3008629, TRACH3008632, TRACH3008688, TRACH3008697, TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037067, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000186, UMVEN2000046, UMVEN2000069, UMVEN2000121, UMVEN2000133, UMVEN2000354, UTERU1000008, UTERU1000015, UTERU1000024, UTERU1000031, UTERU1000032, UTERU1000057, UTERU1000065, UTERU1000077, UTERU1000093, UTERU1000096, UTERU1000109, UTERU1000131, UTERU1000138, UTERU1000148, UTERU1000160, UTERU1000183, UTERU1000187, UTERU1000192, UTERU1000249, UTERU1000337, UTERU1000339, UTERU1000384, UTERU2000023, UTERU2000047, UTERU2000074, UTERU2000099, UTERU2000154, UTERU2000218, UTERU2000238, UTERU2000243, UTERU2000260, UTERU2000263, UTERU2000329, UTERU2000332, UTERU2000338, UTERU2000349, UTERU2000377, UTERU2000393, UTERU2000418, UTERU2000465, UTERU2000485, UTERU2000517, UTERU2000524, UTERU2000537, UTERU2000539, UTERU2000541, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002198, UTERU2002332, UTERU2002410, UTERU2002473, UTERU2002547, UTERU2002693, UTERU2002733, UTERU2002736, UTERU2002737, UTERU2002826, UTERU2002841, UTERU2002964, UTERU2002993, UTERU2003035, UTERU2003057, UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004037, UTERU2004039, UTERU2004061, UTERU2004073, UTERU2004163, UTERU2004197, UTERU2004299, UTERU2004520, UTERU2004564, UTERU2004.688, UTERU2004698, UTERU2004861, UTERU2005004, UTERU2005050, UTERU2005069, UTERU2005074, UTERU2005179, UTERU2005292, UTERU2005346, UTERU2005354, UTERU2005446, UTERU2005449, UTERU2005450, UTERU2005533, UTERU2005593, UTERU2005601, UTERU2005664, UTERU2005822, UTERU2005903, UTERU2005905, UTERU2006103, UTERU2006115, UTERU2006182, UTERU2006400, UTERU2006412, UTERU2006429, UTERU2006486, UTERU2006524, UTERU2006547, UTERU2006568, UTERU2006593, UTERU2006643, UTERU2006651, UTERU2006705, UTERU2006899, UTERU2007004, UTERU2007075, UTERU2007081, UTERU2007128, UTERU2007267, UTERU2007444, UTERU2007499, UTERU2007639, UTERU2007724, UTERU2007924, UTERU2007942, UTERU2008018, UTERU2008019, UTERU2008027, UTERU2008040, UTERU2008077, UTERU2008130, UTERU2008302, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008938, UTERU2008939, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009538, UTERU2009540, UTERU2009776, UTERU2009904, UTERU2009951, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011962, UTERU2011968, UTERU2012031, UTERU2012101, UTERU2012114, UTERU2012252, UTERU2012333, UTERU2012407, UTERU2012526, UTERU2012581, UTERU2012610, UTERU2012615, UTERU2012688, UTERU2012703, UTERU2012715, UTERU2012741, UTERU2012767, UTERU2012786, UTERU2012856, UTERU2012890, UTERU2012938, UTERU2012976, UTERU2013048, UTERU2013231, UTERU2013262, UTERU2013280, UTERU2013322, UTERU2013483, UTERU2013491, UTERU2013502, UTERU2013926, UTERU2013976, UTERU2014001, UTERU2014024, UTERU2014223, UTERU2014398, UTERU2014601, UTERU2014631, UTERU2014678, UTERU2014728, UTERU2014898, UTERU2014998, UTERU2015062, UTERU2015087, UTERU2015108, UTERU2015190, UTERU2015198, UTERU2015202, UTERU2015405, UTERU2015640, UTERU2015830, UTERU2015880, UTERU2016147, UTERU2016157, UTERU2016426, UTERU2016464, UTERU2016669, UTERU2016757, UTERU2016761, UTERU2016822, UTERU2016896, UTERU2016902, UTERU2016979, UTERU2016981, UTERU2017123, UTERU2017303, UTERU2017421, UTERU2017492, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the FEHRT, FEKID, FELIV, FELNG, HCASM, HCHON, and HEART libraries were analyzed for expression frequency. The analysis results are described below. Each is a relative value, where the expression level over all tissues is taken as 100. The larger this value, the greater the level of expression.

TABLE 31

| Clone Name | FEHRT | FEKID | FELIV | FELNG | HCASM | HCHON | HEART |
|---|---|---|---|---|---|---|---|
| 3NB691000129 | 0 | 0 | 0 | 0 | 0 | 15.976 | 0 |
| 3NB691000173 | 0 | 0 | 0 | 0 | 10.485 | 0 | 0 |
| 3NB692000281 | 0 | 4.762 | 0 | 0 | 1.462 | 0 | 0 |
| 3NB692000912 | 0 | 10.163 | 0 | 0 | 0 | 0 | 0 |
| 3NB692001002 | 0 | 42.320 | 0 | 0 | 0 | 0 | 0 |
| 3NB692001022 | 0 | 0 | 0 | 0 | 0 | 7.873 | 0 |
| 3NB692001334 | 0 | 5.806 | 0 | 0 | 0 | 0 | 0 |
| 3NB692001528 | 0 | 50.886 | 0 | 0 | 0 | 0 | 0 |
| ADRGL2000006 | 0 | 0 | 0 | 0 | 0 | 0 | 6.935 |
| ADRGL2000097 | 0 | 0 | 0 | 0 | 0 | 14.068 | 0 |
| ADRGL2010152 | 0 | 0 | 0 | 0 | 0 | 36.653 | 0 |
| ASTRO1000165 | 0 | 0 | 0 | 0 | 14.716 | 0 | 0 |
| ASTRO2000046 | 0 | 0 | 0 | 0 | 0 | 2.582 | 0 |
| ASTRO2000662 | 0 | 0 | 0 | 0 | 53.551 | 0 | 0 |
| ASTRO2001076 | 0 | 0 | 0 | 0 | 9.437 | 0 | 0 |
| ASTRO2001806 | 0 | 0 | 0 | 0 | 9.499 | 0 | 0 |
| ASTRO2001823 | 0 | 0 | 0 | 0 | 0 | 0 | 9.684 |
| ASTRO2003024 | 0 | 0 | 0 | 0 | 0 | 0 | 6.323 |
| ASTRO2003632 | 0 | 0 | 0 | 0 | 0 | 2.629 | 0 |
| ASTRO2013585 | 0 | 0 | 0 | 0 | 3.237 | 3.085 | 0 |
| ASTRO2014863 | 0 | 0 | 0 | 0 | 0 | 21.377 | 0 |
| ASTRO2015328 | 0 | 0 | 0 | 0 | 0 | 24.081 | 0 |
| ASTRO2016491 | 0 | 0 | 0 | 10.830 | 0 | 0 | 0 |
| BGGI12000054 | 0 | 0 | 0 | 0 | 0 | 2.981 | 0 |
| BGGI12000693 | 0 | 40.832 | 0 | 0 | 0 | 0 | 0 |
| BGGI12000839 | 0 | 15.951 | 0 | 0 | 0 | 0 | 0 |
| BGGI12001097 | 0 | 0 | 0 | 0 | 7.461 | 0 | 7.497 |
| BNGH41000177 | 0 | 0 | 0 | 0 | 0 | 0 | 46.383 |
| BNGH42000360 | 0 | 0 | 50.470 | 0 | 0 | 0 | 5.247 |
| BNGH42001247 | 0 | 0 | 0 | 0 | 0 | 33.288 | 0 |
| BNGH42001576 | 0 | 0 | 0 | 0 | 32.396 | 0 | 0 |
| BNGH42005968 | 0 | 0 | 0 | 0 | 15.250 | 0 | 0 |
| BNGH42007460 | 0 | 0 | 0 | 0 | 0 | 6.376 | 0 |
| BNGH42007709 | 0 | 0 | 0 | 0 | 0 | 1.456 | 0 |
| BRACE1000042 | 0 | 0 | 0 | 0 | 0 | 0 | 30.466 |
| BRACE1000070 | 37.227 | 0 | 0 | 0 | 0 | 0 | 0 |
| BRACE1000115 | 0 | 0 | 0 | 0 | 0 | 0 | 1.249 |
| BRACE1000131 | 0 | 0 | 0 | 0 | 0 | 6.591 | 0 |
| BRACE1000186 | 0 | 0 | 0 | 0 | 0 | 13.681 | 0 |
| BRACE2000078 | 0 | 96.823 | 0 | 0 | 0 | 0 | 0 |
| BRACE2000148 | 0 | 0 | 0 | 76.780 | 0 | 0 | 0 |
| BRACE2000307 | 0 | 25.479 | 0 | 0 | 0 | 0 | 0 |
| BRACE2000332 | 0 | 0 | 0 | 0 | 0 | 0 | 18.109 |
| BRACE2001497 | 0 | 0 | 0 | 0 | 0 | 11.791 | 0 |
| BRACE2001855 | 0 | 0 | 0 | 49.493 | 0 | 0 | 0 |
| BRACE2001859 | 0 | 0 | 0 | 0 | 90.342 | 0 | 0 |
| BRACE2001971 | 0 | 0 | 0 | 0 | 14.892 | 0 | 0 |
| BRACE2002392 | 0 | 0 | 0 | 0 | 11.444 | 0 | 0 |
| BRACE2003172 | 0 | 0 | 0 | 0 | 4.316 | 0 | 0 |
| BRACE2003304 | 0 | 47.291 | 0 | 0 | 0 | 0 | 0 |
| BRACE2003825 | 0 | 0 | 0 | 0 | 90.342 | 0 | 0 |
| BRACE2003827 | 0 | 0 | 0 | 0 | 5.817 | 5.544 | 0 |
| BRACE2005087 | 0 | 0 | 0 | 0 | 0 | 6.564 | 0 |
| BRACE2005363 | 0 | 0 | 0 | 0 | 0 | 89.914 | 0 |
| BRACE2005937 | 0 | 0 | 0 | 0 | 40.104 | 0 | 0 |
| BRACE2006537 | 0 | 0 | 0 | 0 | 7.146 | 0 | 0 |
| BRACE2006900 | 0 | 11.130 | 0 | 0 | 0 | 0 | 0 |
| BRACE2006944 | 0 | 0 | 0 | 0 | 0 | 0 | 77.811 |
| BRACE2007798 | 0 | 0 | 0 | 0 | 0 | 0 | 13.801 |
| BRACE2008653 | 0 | 0 | 0 | 0 | 0 | 0 | 3.518 |
| BRACE2008968 | 0 | 0 | 0 | 12.505 | 3.861 | 0 | 0 |
| BRACE2009037 | 0 | 0 | 0 | 0 | 1.939 | 5.543 | 0 |
| BRACE2010336 | 0 | 0 | 0 | 0 | 90.342 | 0 | 0 |
| BRACE2012528 | 0 | 0 | 0 | 0 | 0 | 0 | 6.328 |
| BRACE2012814 | 0 | 33.171 | 0 | 0 | 10.181 | 0 | 10.230 |
| BRACE2013855 | 0 | 0 | 0 | 0 | 0 | 0 | 53.511 |
| BRACE2017397 | 0 | 0 | 0 | 0 | 0 | 14.924 | 0 |
| BRACE2018337 | 0 | 0 | 0 | 0 | 0 | 0 | 15.890 |
| BRACE2039624 | 0 | 0 | 0 | 0 | 0 | 6.122 | 0 |
| BRACE3004371 | 0 | 0 | 0 | 0 | 0 | 0 | 25.063 |
| BRALZ2009482 | 0 | 0 | 0 | 0 | 0 | 0 | 2.479 |
| BRAMY2000354 | 0 | 29.725 | 0 | 0 | 0 | 0 | 0 |
| BRAMY2000411 | 0 | 0 | 0 | 0 | 41.151 | 0 | 0 |
| BRAMY2007287 | 0 | 0 | 0 | 0 | 0 | 0 | 71.484 |
| BRAMY2012536 | 0 | 0 | 0 | 0 | 0 | 0 | 3.851 |
| BRAMY2016325 | 0 | 0 | 0 | 0 | 0 | 23.841 | 0 |
| BRAMY2020427 | 12.330 | 0 | 0 | 12.734 | 0 | 0 | 0 |
| BRAMY2024827 | 0 | 43.154 | 0 | 0 | 0 | 0 | 0 |
| BRAMY2027739 | 0 | 0 | 0 | 28.942 | 0 | 0 | 0 |
| BRAMY2028740 | 0 | 20.139 | 0 | 0 | 0 | 0 | 0 |
| BRAMY2028796 | 30.954 | 0 | 0 | 0 | 9.869 | 0 | 0 |
| BRAMY2040095 | 0 | 0 | 0 | 0 | 2.270 | 0 | 0 |
| BRAMY2040592 | 0 | 0 | 0 | 0 | 0 | 0 | 23.231 |
| BRAMY3004377 | 0 | 0 | 0 | 0 | 15.507 | 0 | 0 |
| BRAMY3010008 | 0 | 12.590 | 0 | 0 | 0 | 0 | 0 |
| BRAWH1000037 | 0 | 0 | 0 | 0 | 0 | 0 | 12.165 |
| BRAWH1000040 | 0 | 0 | 0 | 0 | 0 | 4.354 | 0 |
| BRAWH2000169 | 0 | 0 | 0 | 0 | 69.985 | 0 | 0 |
| BRAWH2000177 | 0 | 0 | 0 | 0 | 0 | 50.592 | 0 |
| BRAWH2000588 | 0 | 0 | 0 | 0 | 8.330 | 0 | 0 |
| BRAWH2000636 | 0 | 0 | 0 | 11.365 | 0 | 0 | 0 |
| BRAWH2000839 | 0 | 0 | 0 | 8.833 | 0 | 0 | 0 |
| BRAWH2000924 | 5.976 | 0 | 0 | 0 | 1.905 | 0 | 0 |
| BRAWH2001019 | 0 | 0 | 0 | 0 | 0 | 0 | 15.215 |
| BRAWH2001092 | 3.660 | 0 | 0 | 0 | 0 | 1.112 | 1.173 |
| BRAWH2001671 | 0 | 0 | 0 | 0 | 0 | 0 | 25.367 |
| BRAWH2002963 | 0 | 0 | 0 | 0 | 0 | 0 | 79.600 |
| BRAWH2005068 | 0 | 8.701 | 0 | 0 | 0 | 2.545 | 0 |
| BRAWH2008706 | 0 | 0 | 0 | 0 | 19.092 | 0 | 0 |
| BRAWH2009304 | 0 | 0 | 0 | 0 | 0 | 0 | 26.166 |
| BRAWH2009307 | 0 | 0 | 0 | 0 | 0 | 0 | 13.283 |
| BRAWH2012077 | 0 | 0 | 0 | 0 | 0 | 79.280 | 0 |
| BRAWH2014645 | 0 | 0 | 0 | 0 | 7.019 | 0 | 0 |
| BRAWH2017685 | 0 | 10.978 | 0 | 0 | 3.370 | 0 | 0 |
| BRAWH2018526 | 0 | 0 | 0 | 0 | 2.043 | 0 | 2.053 |
| BRAWH3002853 | 0 | 0 | 0 | 0 | 0 | 4.680 | 0 |
| BRAWH3024989 | 0 | 0 | 0 | 0 | 15.730 | 0 | 0 |
| BRCAN1000168 | 0 | 0 | 0 | 0 | 0 | 0 | 9.411 |
| BRCAN2002173 | 0 | 0 | 0 | 42.766 | 0 | 0 | 0 |
| BRCAN2003814 | 90.076 | 0 | 0 | 0 | 0 | 0 | 0 |
| BRCAN2003944 | 0 | 0 | 0 | 0 | 1.870 | 3.564 | 0 |
| BRCAN2026197 | 0 | 0 | 0 | 0 | 10.366 | 0 | 0 |
| BRCAN2028036 | 0 | 0 | 0 | 0 | 16.030 | 0 | 0 |
| BRCOC1000094 | 0 | 0 | 0 | 0 | 6.322 | 0 | 0 |
| BRCOC2003187 | 0 | 34.023 | 0 | 0 | 0 | 0 | 0 |
| BRCOC2018775 | 0 | 0 | 0 | 0 | 0 | 64.108 | 0 |
| BRHIP2000534 | 0 | 0 | 0 | 0 | 2.272 | 2.166 | 0 |
| BRHIP2007305 | 0 | 0 | 0 | 0 | 7.516 | 0 | 0 |
| BRHIP2011491 | 0 | 0 | 0 | 0 | 0 | 0 | 5.514 |
| BRHIP2022326 | 0 | 36.805 | 0 | 0 | 5.648 | 0 | 11.351 |
| BRHIP2023888 | 0 | 0 | 0 | 0 | 2.443 | 2.328 | 0 |
| BRSSN2015493 | 0 | 0 | 0 | 78.612 | 0 | 0 | 0 |
| BRSTN2011961 | 0 | 6.265 | 0 | 0 | 1.923 | 0 | 1.932 |
| BRSTN2012069 | 0.394 | 1.841 | 0 | 2.238 | 0.377 | 0.180 | 1.641 |
| BRSTN2012174 | 0 | 8.750 | 0 | 4.350 | 0 | 0 | 2.699 |
| BRSTN2016470 | 0 | 0 | 0 | 4.707 | 0 | 0 | 1.460 |
| BRSTN2016678 | 0 | 0 | 0 | 0 | 60.919 | 0 | 0 |
| BRSTN2016992 | 0 | 0 | 0 | 0 | 0 | 0 | 6.566 |
| BRTHA2012714 | 0 | 0 | 0 | 0 | 0 | 15.862 | 16.724 |
| BRTHA2027229 | 0 | 19.799 | 0 | 0 | 0 | 0 | 0 |
| CD34C3000252 | 0 | 0 | 0 | 0 | 5.449 | 0 | 0 |
| CTONG1000087 | 0 | 0 | 0 | 0 | 0 | 6.617 | 0 |
| CTONG1000175 | 0 | 0 | 0 | 36.210 | 0 | 0 | 0 |
| CTONG2000452 | 0 | 0 | 0 | 0 | 19.337 | 0 | 0 |
| CTONG2001748 | 0 | 0 | 0 | 0 | 0 | 13.227 | 0 |
| CTONG2002073 | 0 | 0 | 0 | 0 | 0 | 9.268 | 0 |
| CTONG2002520 | 0 | 0 | 0 | 0 | 19.545 | 0 | 0 |
| CTONG2003298 | 0 | 0 | 0 | 0 | 4.818 | 4.592 | 0 |
| CTONG2006004 | 0 | 0 | 0 | 0 | 0 | 0 | 7.042 |
| CTONG2006562 | 0 | 30.286 | 0 | 0 | 0 | 0 | 0 |
| CTONG2006932 | 0 | 0 | 0 | 23.667 | 0 | 0 | 0 |
| CTONG2008506 | 0 | 20.356 | 0 | 0 | 0 | 5.954 | 0 |
| CTONG2010821 | 0 | 0 | 0 | 0 | 0 | 4.522 | 0 |
| CTONG2012452 | 0 | 0 | 0 | 0 | 0 | 0 | 2.376 |
| CTONG2012564 | 0 | 85.462 | 0 | 0 | 0 | 0 | 0 |
| CTONG2012745 | 0 | 0 | 0 | 0 | 36.827 | 0 | 0 |
| CTONG2013907 | 0 | 0 | 0 | 0 | 0 | 13.642 | 0 |
| CTONG2015091 | 0 | 0 | 0 | 0 | 0 | 0 | 5.418 |
| CTONG2016575 | 0 | 0 | 0 | 0 | 3.301 | 0 | 0 |
| CTONG2016824 | 0 | 0 | 0 | 0 | 0 | 26.934 | 0 |
| CTONG2016931 | 0 | 0 | 0 | 0 | 0 | 0 | 20.359 |
| CTONG2017500 | 55.163 | 0 | 0 | 0 | 0 | 0 | 0 |
| CTONG2018900 | 0 | 0 | 0 | 92.030 | 0 | 0 | 0 |
| CTONG2020484 | 0 | 0 | 0 | 0 | 0 | 3.720 | 0 |
| CTONG2028124 | 0 | 0 | 0 | 10.809 | 0 | 0 | 3.353 |
| CTONG2028208 | 0 | 0 | 0 | 0 | 23.511 | 0 | 0 |
| CTONG3000657 | 0 | 0 | 0 | 0 | 0 | 0 | 25.884 |
| CTONG3009028 | 0 | 0 | 0 | 0 | 10.685 | 0 | 0 |
| D3OST3000169 | 10.191 | 0 | 0 | 0 | 0 | 0 | 0 |
| D6OST2000464 | 0 | 0 | 0 | 0 | 7.274 | 0 | 0 |
| D9OST2003397 | 0 | 0 | 0 | 61.464 | 0 | 0 | 0 |
| DFNES2000011 | 0 | 0 | 0 | 0 | 7.261 | 0 | 0 |
| DFNES2002509 | 0 | 0 | 0 | 0 | 0 | 32.128 | 0 |
| DFNES2005540 | 0 | 0 | 0 | 0 | 0 | 19.128 | 20.167 |
| DFNES2005766 | 0 | 0 | 0 | 0 | 0 | 11.601 | 0 |
| DFNES2007332 | 0 | 0 | 0 | 0 | 0 | 0 | 49.518 |
| FCBBF1000027 | 0 | 0 | 0 | 0 | 2.432 | 0 | 4.887 |
| FCBBF1000139 | 0 | 0 | 0 | 0 | 0 | 11.061 | 0 |
| FCBBF1000280 | 0 | 0 | 0 | 0 | 0 | 35.741 | 0 |
| FCBBF1000367 | 0 | 0 | 0 | 0 | 0 | 9.613 | 0 |
| FCBBF1000732 | 0 | 0 | 0 | 0 | 18.591 | 0 | 0 |
| FCBBF2001291 | 0 | 41.153 | 0 | 0 | 12.631 | 0 | 0 |
| FCBBF2001538 | 0 | 0 | 0 | 0 | 5.059 | 0 | 0 |
| FCBBF2005658 | 0 | 0 | 0 | 0 | 51.674 | 0 | 0 |
| FCBBF3001470 | 0 | 0 | 0 | 0 | 0 | 8.589 | 0 |
| FCBBF3001632 | 0 | 33.393 | 0 | 0 | 0 | 0 | 0 |
| FCBBF3003800 | 0 | 0 | 0 | 0 | 2.615 | 2.493 | 2.628 |
| FCBBF3005320 | 0 | 0 | 0 | 0 | 0 | 0 | 18.199 |
| FCBBF3005330 | 14.426 | 14.985 | 0 | 0 | 0 | 0 | 0 |
| FCBBF3007244 | 0 | 0 | 0 | 0 | 0 | 49.116 | 0 |
| FCBBF3009541 | 0 | 0 | 0 | 0 | 0 | 2.608 | 0 |
| FCBBF3016928 | 0 | 0 | 0 | 18.845 | 0 | 0 | 0 |
| FCBBF3019455 | 0 | 0 | 0 | 0 | 54.072 | 0 | 0 |
| FCBBF3019714 | 0 | 0 | 0 | 0 | 0 | 12.961 | 0 |
| FCBBF3022894 | 91.851 | 0 | 0 | 0 | 0 | 0 | 0 |
| FCBBF3023078 | 0 | 0 | 0 | 92.090 | 0 | 0 | 0 |
| FCBBF3023368 | 0 | 0 | 0 | 0 | 16.290 | 15.525 | 0 |
| FCBBF3023667 | 0 | 2.873 | 0 | 0 | 0 | 0.840 | 1.772 |
| FCBBF4000017 | 0 | 0 | 0 | 0 | 0 | 56.201 | 0 |
| FCBBF4000446 | 0 | 0 | 0 | 0 | 4.987 | 0 | 0 |
| FEBRA2000129 | 0 | 0 | 0 | 0 | 17.260 | 0 | 0 |
| FEBRA2000733 | 0 | 0 | 0 | 0 | 2.238 | 1.066 | 2.249 |
| FEBRA2000740 | 0 | 0 | 0 | 0 | 7.509 | 0 | 0 |
| FEBRA2000787 | 0 | 0 | 0 | 42.481 | 0 | 0 | 0 |
| FEBRA2001294 | 0 | 0 | 0 | 0 | 16.045 | 0 | 0 |
| FEBRA2002456 | 0 | 0 | 0 | 0 | 0 | 6.902 | 0 |
| FEBRA2002933 | 0 | 0 | 0 | 0 | 2.457 | 0 | 2.469 |
| FEBRA2003926 | 0 | 0 | 0 | 0 | 4.635 | 0 | 0 |
| FEBRA2004224 | 0 | 0 | 0 | 0 | 0 | 7.326 | 0 |
| FEBRA2005572 | 0 | 0 | 0 | 0 | 0 | 0 | 11.887 |
| FEBRA2005734 | 17.583 | 0 | 0 | 0 | 0 | 0 | 0 |
| FEBRA2007622 | 0 | 0 | 0 | 0 | 0 | 13.056 | 0 |
| FEBRA2007880 | 0 | 0 | 0 | 0 | 0 | 0 | 25.109 |
| FEBRA2008087 | 0 | 0 | 0 | 0 | 2.380 | 0 | 2.392 |
| FEBRA2008287 | 61.381 | 0 | 0 | 0 | 0 | 0 | 0 |
| FEBRA2008583 | 0 | 0 | 0 | 0 | 0 | 0.836 | 0 |
| FEBRA2008755 | 0 | 0 | 0 | 0 | 0 | 10.394 | 0 |
| FEBRA2009419 | 0 | 0 | 0 | 0 | 8.773 | 0 | 0 |
| FEBRA2014417 | 0 | 0 | 0 | 0 | 0 | 3.416 | 0 |
| FEBRA2020566 | 0 | 0 | 0 | 89.613 | 0 | 0 | 0 |
| FEBRA2022055 | 0 | 0 | 0 | 0 | 0 | 0 | 7.100 |
| FEBRA2022504 | 0 | 0 | 0 | 0 | 0 | 31.177 | 0 |
| FEBRA2023399 | 0 | 0 | 0 | 0 | 0 | 55.540 | 0 |
| FEBRA2023927 | 0 | 0 | 0 | 0 | 0 | 0 | 27.172 |
| FEBRA2025427 | 0 | 0 | 0 | 0 | 0 | 15.685 | 0 |
| FEBRA2025838 | 0 | 17.770 | 0 | 0 | 0 | 0 | 0 |
| FEHRT2000325 | 100 | 0 | 0 | 0 | 0 | 0 | 0 |
| FEHRT2000364 | 92.290 | 0 | 0 | 0 | 0 | 0 | 0 |
| FEKID1000018 | 0 | 85.388 | 0 | 0 | 0 | 0 | 0 |
| FELIV1000101 | 0 | 0 | 99.741 | 0 | 0 | 0 | 0 |
| FELIV1000137 | 0 | 0 | 99.433 | 0 | 0 | 0 | 0 |
| FELIV1000153 | 0 | 0 | 52.921 | 0 | 1.095 | 1.044 | 0 |
| FELIV1000164 | 6.055 | 0 | 93.294 | 0 | 0 | 0 | 0 |
| FELNG2000241 | 0 | 0 | 0 | 100 | 0 | 0 | 0 |
| HCASM1000021 | 0 | 0 | 0 | 0 | 100 | 0 | 0 |
| HCASM1000050 | 0 | 0 | 0 | 0 | 100 | 0 | 0 |
| HCASM1000061 | 0 | 0 | 0 | 0 | 100 | 0 | 0 |
| HCASM1000115 | 0 | 0 | 0 | 0 | 6.882 | 0 | 0 |
| HCASM1000130 | 0 | 0 | 0 | 0 | 100 | 0 | 0 |
| HCASM2000016 | 0 | 0 | 0 | 0 | 4.955 | 9.445 | 0 |
| HCASM2000138 | 0 | 0 | 0 | 0 | 28.042 | 0 | 0 |
| HCASM2000156 | 0 | 0 | 0 | 0 | 86.824 | 0 | 0 |
| HCASM2000202 | 0 | 0 | 0 | 0 | 100 | 0 | 0 |
| HCASM2000214 | 0 | 0 | 0 | 0 | 62.795 | 0 | 0 |
| HCASM2000266 | 0 | 0 | 0 | 0 | 100 | 0 | 0 |
| HCASM2000307 | 0 | 0 | 0 | 0 | 100 | 0 | 0 |
| HCASM2000363 | 0 | 0 | 0 | 0 | 9.030 | 0 | 0 |
| HCASM2000534 | 0 | 0 | 0 | 0 | 100 | 0 | 0 |
| HCASM2000536 | 0 | 0 | 0 | 0 | 100 | 0 | 0 |
| HCASM2000560 | 0 | 0 | 0 | 0 | 100 | 0 | 0 |
| HCASM2000871 | 0 | 0 | 0 | 0 | 100 | 0 | 0 |
| HCASM2001285 | 0 | 0 | 0 | 0 | 100 | 0 | 0 |
| HCASM2001810 | 0 | 0 | 0 | 0 | 100 | 0 | 0 |
| HCASM2001866 | 0 | 0 | 0 | 0 | 100 | 0 | 0 |
| HCASM2001890 | 0 | 0 | 0 | 0 | 84.772 | 0 | 0 |
| HCASM2002148 | 0 | 0 | 0 | 0 | 100 | 0 | 0 |
| HCASM2002502 | 0 | 0 | 0 | 0 | 100 | 0 | 0 |
| HCASM2002754 | 0 | 0 | 0 | 0 | 100 | 0 | 0 |
| HCASM2002918 | 0 | 0 | 0 | 0 | 100 | 0 | 0 |
| HCASM2003018 | 0 | 0 | 0 | 0 | 58.599 | 0 | 0 |
| HCASM2003076 | 0 | 0 | 0 | 0 | 37.805 | 36.029 | 0 |
| HCASM2003099 | 0 | 0 | 0 | 0 | 100 | 0 | 0 |
| HCASM2003212 | 0 | 0 | 0 | 0 | 100 | 0 | 0 |
| HCASM2003237 | 0 | 0 | 0 | 0 | 100 | 0 | 0 |
| HCASM2003357 | 0 | 0 | 0 | 0 | 100 | 0 | 0 |
| HCASM2003415 | 0 | 36.285 | 0 | 0 | 11.137 | 0 | 0 |
| HCASM2005388 | 0 | 0 | 0 | 0 | 23.484 | 0 | 0 |
| HCASM2005484 | 0 | 0 | 0 | 0 | 85.688 | 0 | 0 |
| HCASM2006131 | 0 | 0 | 0 | 0 | 100 | 0 | 0 |
| HCASM2006338 | 0 | 0 | 0 | 0 | 100 | 0 | 0 |
| HCASM2006359 | 0 | 0 | 0 | 0 | 100 | 0 | 0 |
| HCASM2006632 | 0 | 0 | 0 | 0 | 100 | 0 | 0 |
| HCASM2007047 | 0 | 0 | 0 | 73.789 | 22.780 | 0 | 0 |
| HCASM2007317 | 0 | 0 | 0 | 0 | 100 | 0 | 0 |
| HCASM2007737 | 0 | 0 | 0 | 0 | 100 | 0 | 0 |
| HCASM2007773 | 0 | 0 | 0 | 0 | 100 | 0 | 0 |
| HCASM2008154 | 0 | 0 | 0 | 0 | 100 | 0 | 0 |
| HCASM2008536 | 0 | 0 | 0 | 0 | 84.772 | 0 | 0 |
| HCASM2009405 | 0 | 0 | 0 | 0 | 100 | 0 | 0 |
| HCASM2009424 | 0 | 0 | 0 | 0 | 100 | 0 | 0 |
| HCASM2009463 | 0 | 0 | 0 | 0 | 100 | 0 | 0 |
| HCASM2009588 | 0 | 0 | 0 | 0 | 6.946 | 0 | 0 |
| HCHON1000015 | 0 | 0 | 0 | 0 | 0 | 9.863 | 10.399 |
| HCHON1000030 | 3.777 | 0 | 0 | 0 | 0 | 1.148 | 2.420 |
| HCHON1000131 | 0 | 0 | 0 | 0 | 0 | 25.961 | 0 |
| HCHON1000142 | 0 | 0 | 0 | 0 | 0 | 84.140 | 0 |
| HCHON1000166 | 0 | 0 | 0 | 0 | 0 | 19.035 | 20.069 |
| HCHON1000176 | 0 | 0 | 0 | 0 | 0 | 100 | 0 |
| HCHON1000191 | 10.058 | 0 | 0 | 0 | 0 | 9.169 | 0 |
| HCHON2000028 | 0 | 0 | 0 | 0 | 0 | 10.039 | 0 |
| HCHON2000038 | 0 | 0 | 0 | 0 | 0 | 100 | 0 |
| HCHON2000056 | 0 | 0 | 0 | 0 | 1.883 | 5.384 | 1.892 |
| HCHON2000062 | 0 | 0 | 0 | 0 | 0 | 26.520 | 0 |
| HCHON2000087 | 0 | 0 | 0 | 0 | 0 | 3.915 | 4.127 |
| HCHON2000100 | 0 | 0 | 0 | 0 | 0 | 100 | 0 |
| HCHON2000156 | 0 | 0 | 0 | 0 | 0 | 100 | 0 |
| HCHON2000160 | 0 | 0 | 0 | 0 | 0 | 6.369 | 0 |
| HCHON2000199 | 0 | 0 | 0 | 0 | 0 | 100 | 0 |
| HCHON2000212 | 0 | 0 | 0 | 0 | 0 | 54.383 | 0 |
| HCHON2000226 | 0 | 8.723 | 0 | 8.672 | 2.677 | 5.103 | 0 |
| HCHON2000244 | 13.698 | 0 | 0 | 0 | 0 | 4.162 | 0 |
| HCHON2000265 | 0 | 0 | 0 | 0 | 0 | 12.802 | 0 |
| HCHON2000271 | 0 | 0 | 0 | 0 | 0 | 100 | 0 |
| HCHON2000295 | 0 | 0 | 0 | 0 | 0 | 2.772 | 0 |
| HCHON2000315 | 0 | 0 | 0 | 0 | 0 | 100 | 0 |
| HCHON2000322 | 0 | 0 | 0 | 0 | 0 | 69.478 | 0 |
| HCHON2000323 | 0 | 0 | 0 | 0 | 12.299 | 11.721 | 0 |
| HCHON2000344 | 0 | 0 | 0 | 0 | 0 | 100 | 0 |
| HCHON2000364 | 0 | 0 | 0 | 0 | 2.952 | 28.131 | 0 |
| HCHON2000373 | 0 | 0 | 0 | 0 | 6.729 | 19.239 | 0 |
| HCHON2000395 | 0 | 0 | 0 | 0 | 0 | 100 | 0 |
| HCHON2000417 | 0 | 0 | 0 | 0 | 0 | 100 | 0 |
| HCHON2000418 | 0 | 0 | 0 | 0 | 0 | 84.140 | 0 |
| HCHON2000473 | 0 | 0 | 0 | 0 | 0 | 37.038 | 0 |
| HCHON2000475 | 0 | 0 | 0 | 0 | 0 | 8.423 | 0 |
| HCHON2000503 | 0 | 0 | 0 | 0 | 0 | 100 | 0 |
| HCHON2000508 | 0 | 0 | 0 | 0 | 2.479 | 4.726 | 0 |
| HCHON2000626 | 0 | 0 | 0 | 0 | 7.805 | 14.876 | 7.842 |
| HCHON2000660 | 0 | 0 | 0 | 0 | 0 | 100 | 0 |
| HCHON2000663 | 0 | 0 | 0 | 0 | 0 | 53.978 | 0 |
| HCHON2000673 | 0 | 0 | 0 | 0 | 0 | 100 | 0 |
| HCHON2000676 | 0 | 0 | 0 | 0 | 19.795 | 18.865 | 0 |
| HCHON2000698 | 0 | 0 | 0 | 0 | 0 | 18.136 | 0 |
| HCHON2000699 | 0 | 0 | 0 | 0 | 0 | 88.356 | 0 |
| HCHON2000705 | 0 | 0 | 0 | 0 | 0 | 63.051 | 0 |
| HCHON2000738 | 0 | 0 | 0 | 0 | 0 | 18.841 | 0 |
| HCHON2000743 | 0 | 0 | 0 | 0 | 0 | 38.633 | 0 |
| HCHON2000751 | 0 | 0 | 0 | 0 | 0 | 22.591 | 23.818 |
| HCHON2000815 | 0 | 0 | 0 | 0 | 0 | 35.342 | 0 |
| HCHON2000817 | 0 | 0 | 0 | 0 | 0 | 100 | 0 |
| HCHON2000818 | 0 | 0 | 0 | 0 | 0 | 100 | 0 |
| HCHON2000819 | 0 | 0 | 0 | 0 | 0 | 46.359 | 0 |
| HCHON2000826 | 0 | 0 | 0 | 0 | 0 | 4.529 | 0 |
| HCHON2000832 | 0 | 0 | 0 | 0 | 0 | 22.386 | 0 |
| HCHON2000851 | 0 | 0 | 0 | 0 | 0 | 100 | 0 |
| HCHON2000898 | 0 | 0 | 0 | 0 | 0 | 64.476 | 0 |
| HCHON2000935 | 0 | 0 | 0 | 0 | 0 | 100 | 0 |
| HCHON2000942 | 0 | 0 | 0 | 0 | 0 | 71.738 | 0 |
| HCHON2000956 | 0 | 0 | 0 | 0 | 0 | 100 | 0 |
| HCHON2000994 | 0 | 0 | 0 | 0 | 0 | 100 | 0 |
| HCHON2001039 | 0 | 0 | 0 | 0 | 7.408 | 7.061 | 0 |
| HCHON2001050 | 0 | 0 | 0 | 0 | 9.623 | 9.171 | 0 |
| HCHON2001084 | 0 | 0 | 0 | 0 | 0 | 72.645 | 0 |
| HCHON2001099 | 0 | 0 | 0 | 0 | 0 | 34.516 | 0 |
| HCHON2001108 | 0 | 0 | 0 | 0 | 0 | 13.511 | 0 |
| HCHON2001110 | 0 | 0 | 0 | 0 | 0 | 52.199 | 0 |
| HCHON2001116 | 0 | 0 | 0 | 0 | 0 | 100 | 0 |
| HCHON2001161 | 0 | 0 | 0 | 0 | 0 | 68.132 | 0 |
| HCHON2001200 | 0 | 0 | 0 | 0 | 0 | 2.737 | 0 |
| HCHON2001214 | 0 | 0 | 0 | 0 | 0 | 100 | 0 |
| HCHON2001217 | 0 | 0 | 0 | 0 | 6.911 | 6.586 | 0 |
| HCHON2001233 | 0 | 0 | 0 | 0 | 0 | 100 | 0 |
| HCHON2001269 | 0 | 0 | 0 | 0 | 0 | 100 | 0 |
| HCHON2001359 | 0 | 0 | 0 | 0 | 0 | 5.387 | 0 |
| HCHON2001407 | 0 | 0 | 0 | 16.983 | 0 | 4.997 | 0 |
| HCHON2001434 | 0 | 0 | 0 | 0 | 0 | 9.666 | 10.191 |
| HCHON2001450 | 0 | 0 | 0 | 0 | 0 | 100 | 0 |
| HCHON2001453 | 0 | 0 | 0 | 0 | 0 | 100 | 0 |
| HCHON2001497 | 0 | 0 | 0 | 0 | 0 | 47.410 | 0 |
| HCHON2001505 | 0 | 0 | 0 | 0 | 0 | 28.434 | 0 |
| HCHON2001519 | 0 | 0 | 0 | 0 | 0 | 90.637 | 0 |
| HCHON2001523 | 0 | 0 | 0 | 0 | 0 | 100 | 0 |
| HCHON2001535 | 0 | 0 | 0 | 0 | 0 | 58.352 | 0 |
| HCHON2001546 | 0 | 0 | 0 | 0 | 0 | 45.846 | 0 |
| HCHON2001548 | 0 | 0 | 0 | 0 | 0 | 72.417 | 0 |
| HCHON2001577 | 0 | 0 | 0 | 0 | 0 | 100 | 0 |
| HCHON2001598 | 0 | 0 | 0 | 0 | 0 | 15.375 | 0 |
| HCHON2001604 | 0 | 0 | 0 | 0 | 0 | 100 | 0 |
| HCHON2001607 | 0 | 0 | 0 | 0 | 0 | 100 | 0 |
| HCHON2001646 | 0 | 0 | 0 | 0 | 0 | 100 | 0 |
| HCHON2001665 | 0 | 0 | 0 | 33.188 | 0 | 9.764 | 0 |
| HCHON2001712 | 0 | 0 | 0 | 0 | 0 | 88.356 | 0 |
| HCHON2001768 | 0 | 0 | 0 | 0 | 0 | 100 | 0 |
| HCHON2001801 | 0 | 0 | 0 | 0 | 0 | 64.108 | 0 |
| HCHON2001853 | 0 | 0 | 0 | 0 | 0 | 35.438 | 0 |
| HCHON2002123 | 0 | 0 | 0 | 0 | 0 | 100 | 0 |
| HCHON2002194 | 0 | 0 | 0 | 0 | 0 | 100 | 0 |
| HCHON2002241 | 0 | 0 | 0 | 0 | 0 | 100 | 0 |
| HCHON2002247 | 0 | 0 | 0 | 0 | 0 | 17.764 | 0 |
| HCHON2002354 | 0 | 0 | 0 | 0 | 0 | 80.389 | 0 |
| HCHON2002496 | 0 | 0 | 0 | 0 | 0 | 49.148 | 0 |
| HCHON2002676 | 0 | 0 | 0 | 0 | 0 | 86.353 | 0 |
| HCHON2002770 | 0 | 0 | 0 | 0 | 0 | 100 | 0 |
| HCHON2002964 | 0 | 0 | 0 | 0 | 0 | 100 | 0 |
| HCHON2002971 | 0 | 0 | 0 | 0 | 0 | 88.356 | 0 |
| HCHON2003131 | 0 | 0 | 0 | 0 | 0 | 100 | 0 |
| HCHON2003134 | 0 | 0 | 0 | 0 | 0 | 100 | 0 |
| HCHON2003327 | 0 | 0 | 0 | 0 | 0 | 100 | 0 |
| HCHON2003513 | 0 | 0 | 0 | 0 | 0 | 100 | 0 |
| HCHON2003532 | 0 | 0 | 0 | 0 | 0 | 100 | 0 |
| HCHON2003642 | 0 | 0 | 0 | 0 | 0 | 78.436 | 0 |
| HCHON2003659 | 0 | 0 | 0 | 0 | 0 | 100 | 0 |
| HCHON2003676 | 0 | 0 | 0 | 0 | 0 | 100 | 0 |
| HCHON2004002 | 0 | 0 | 0 | 0 | 0 | 4.968 | 0 |
| HCHON2004007 | 0 | 0 | 0 | 0 | 0 | 100 | 0 |
| HCHON2004279 | 0 | 0 | 0 | 0 | 0 | 100 | 0 |
| HCHON2004359 | 0 | 0 | 0 | 0 | 0 | 100 | 0 |
| HCHON2004531 | 0 | 0 | 0 | 0 | 0 | 100 | 0 |
| HCHON2004776 | 0 | 0 | 0 | 0 | 0 | 100 | 0 |
| HCHON2004858 | 0 | 0 | 0 | 0 | 0 | 83.589 | 0 |
| HCHON2005048 | 0 | 0 | 0 | 0 | 0 | 100 | 0 |
| HCHON2005118 | 0 | 0 | 0 | 0 | 0 | 100 | 0 |
| HCHON2005166 | 0 | 0 | 0 | 0 | 0 | 77.401 | 0 |
| HCHON2005802 | 0 | 0 | 0 | 0 | 0 | 100 | 0 |
| HCHON2005921 | 0 | 0 | 0 | 0 | 0 | 26.243 | 0 |
| HCHON2005987 | 0 | 0 | 0 | 0 | 0 | 63.091 | 0 |
| HCHON2006250 | 0 | 0 | 0 | 0 | 0 | 9.901 | 0 |
| HCHON2006459 | 0 | 0 | 0 | 0 | 0 | 68.296 | 0 |
| HCHON2006714 | 0 | 0 | 0 | 0 | 0 | 100 | 0 |
| HCHON2006722 | 0 | 0 | 0 | 0 | 0 | 64.108 | 0 |
| HCHON2006770 | 0 | 0 | 0 | 0 | 0 | 17.858 | 0 |
| HCHON2006786 | 0 | 0 | 0 | 0 | 0 | 86.115 | 0 |
| HCHON2006841 | 0 | 0 | 0 | 0 | 0 | 100 | 0 |
| HCHON2006871 | 0 | 0 | 0 | 0 | 0 | 100 | 0 |
| HCHON2007046 | 0 | 0 | 0 | 77.267 | 0 | 22.733 | 0 |
| HCHON2007482 | 0 | 0 | 0 | 0 | 0 | 100 | 0 |
| HCHON2007650 | 0 | 0 | 0 | 0 | 0 | 77.065 | 0 |
| HCHON2007674 | 0 | 0 | 0 | 0 | 0 | 100 | 0 |
| HCHON2007774 | 0 | 0 | 0 | 0 | 0 | 100 | 0 |
| HCHON2007881 | 0 | 0 | 0 | 0 | 0 | 83.589 | 0 |
| HCHON2008112 | 0 | 0 | 0 | 0 | 0 | 86.264 | 0 |
| HCHON2008239 | 0 | 0 | 0 | 0 | 0 | 100 | 0 |
| HCHON2008444 | 0 | 0 | 0 | 0 | 0 | 84.140 | 0 |
| HCHON2008582 | 0 | 0 | 0 | 0 | 0 | 100 | 0 |
| HCHON2008672 | 0 | 0 | 0 | 0 | 0 | 100 | 0 |
| HCHON2008856 | 0 | 0 | 0 | 0 | 0 | 63.091 | 0 |
| HCHON2008871 | 0 | 0 | 0 | 0 | 0 | 100 | 0 |
| HCHON2008989 | 0 | 0 | 0 | 0 | 0 | 100 | 0 |
| HCHON2009191 | 0 | 0 | 0 | 0 | 0 | 100 | 0 |
| HCHON2009384 | 0 | 0 | 0 | 0 | 0 | 100 | 0 |
| HCHON2009749 | 0 | 0 | 0 | 0 | 0 | 100 | 0 |
| HCHON2009766 | 0 | 0 | 0 | 0 | 0 | 100 | 0 |
| HCHON2009795 | 0 | 0 | 0 | 0 | 0 | 100 | 0 |
| HCHON2010074 | 0 | 0 | 0 | 0 | 0 | 100 | 0 |
| HCHON2010543 | 0 | 0 | 0 | 0 | 0 | 56.763 | 0 |
| HEART1000010 | 0 | 0 | 0 | 0 | 0 | 0 | 22.793 |
| HEART1000074 | 0 | 0 | 0 | 32.652 | 0 | 0 | 5.064 |
| HEART1000088 | 0 | 0 | 0 | 0 | 0 | 0 | 49.759 |
| HEART1000099 | 0 | 0 | 0 | 0 | 0 | 0 | 100 |
| HEART1000102 | 0 | 0 | 0 | 0 | 0 | 0 | 100 |
| HEART1000118 | 0 | 0 | 0 | 0 | 0 | 0 | 73.940 |
| HEART1000132 | 0 | 0 | 0 | 0 | 0 | 0 | 58.129 |
| HEART1000139 | 0 | 0 | 0 | 0 | 0 | 0 | 66.452 |
| HEART1000142 | 37.124 | 0 | 0 | 0 | 0 | 0 | 11.893 |
| HEART1000149 | 0 | 0 | 0 | 0 | 0 | 0 | 6.265 |
| HEART1000151 | 0 | 0 | 0 | 0 | 0 | 0 | 100 |
| HEART1000173 | 0 | 0 | 0 | 0 | 0 | 0 | 17.510 |
| HEART1000185 | 32.926 | 0 | 0 | 0 | 0 | 5.002 | 26.371 |
| HEART2000024 | 0 | 0 | 0 | 0 | 0 | 0 | 100 |
| HEART2000035 | 0 | 0 | 0 | 0 | 0 | 0 | 100 |
| HEART2000099 | 0 | 0 | 0 | 0 | 0 | 0 | 100 |
| HEART2000298 | 0 | 0 | 0 | 0 | 0 | 0 | 100 |
| HEART2000306 | 0 | 0 | 0 | 0 | 0 | 0 | 90.384 |
| HEART2000309 | 0 | 0 | 0 | 0 | 0 | 0 | 100 |
| HEART2000411 | 0 | 0 | 0 | 0 | 0 | 0 | 84.451 |
| HEART2000418 | 0 | 0 | 0 | 0 | 0 | 0 | 100 |
| HEART2000444 | 0 | 0 | 0 | 0 | 0 | 0 | 56.184 |
| HEART2000448 | 0 | 0 | 0 | 0 | 0 | 0 | 100 |
| HEART2000492 | 0 | 0 | 0 | 0 | 0 | 0 | 100 |
| HEART2000506 | 0 | 0 | 0 | 0 | 0 | 0 | 70.197 |
| HEART2000520 | 0 | 0 | 0 | 0 | 0 | 0 | 38.479 |
| HEART2000541 | 0 | 0 | 0 | 0 | 0 | 0 | 53.555 |
| HEART2000568 | 0 | 0 | 0 | 0 | 0 | 0 | 100 |
| HEART2000611 | 0 | 0 | 0 | 0 | 0 | 0 | 100 |
| HEART2000959 | 0 | 0 | 0 | 0 | 0 | 0 | 78.578 |
| HEART2001680 | 0 | 0 | 0 | 0 | 0 | 0 | 100 |
| HEART2001756 | 0 | 0 | 0 | 0 | 0 | 0 | 100 |
| HEART2002129 | 0 | 0 | 0 | 0 | 0 | 0 | 100 |
| HEART2002184 | 0 | 0 | 0 | 0 | 0 | 0 | 100 |
| HEART2002220 | 0 | 0 | 0 | 0 | 0 | 0 | 100 |
| HEART2002432 | 0 | 0 | 0 | 0 | 0 | 0 | 100 |
| HEART2002531 | 0 | 0 | 0 | 0 | 0 | 0 | 100 |
| HEART2002598 | 0 | 0 | 0 | 0 | 0 | 0 | 100 |
| HEART2002717 | 0 | 0 | 0 | 0 | 0 | 0 | 100 |
| HEART2003027 | 0 | 0 | 0 | 0 | 0 | 0 | 100 |
| HEART2003168 | 0 | 0 | 0 | 0 | 0 | 0 | 85.747 |
| HEART2003432 | 0 | 0 | 0 | 0 | 0 | 0 | 100 |
| HEART2003440 | 0 | 0 | 0 | 0 | 0 | 0 | 19.529 |
| HEART2003781 | 0 | 0 | 0 | 0 | 0 | 0 | 100 |
| HEART2003836 | 0 | 0 | 0 | 0 | 0 | 0 | 27.450 |
| HEART2003852 | 0 | 0 | 0 | 0 | 0 | 0 | 25.993 |
| HEART2004570 | 0 | 0 | 0 | 0 | 0 | 0 | 100 |
| HEART2004764 | 0 | 0 | 0 | 0 | 0 | 0 | 83.614 |
| HEART2004931 | 0 | 0 | 0 | 0 | 0 | 0 | 45.365 |
| HEART2004940 | 0 | 0 | 0 | 0 | 0 | 0 | 100 |
| HEART2004941 | 0 | 0 | 0 | 0 | 0 | 0 | 63.375 |
| HEART2004980 | 0 | 0 | 0 | 0 | 0 | 0 | 100 |
| HEART2005244 | 0 | 0 | 0 | 0 | 0 | 0 | 100 |
| HEART2005310 | 0 | 0 | 0 | 0 | 0 | 0 | 46.510 |
| HEART2005581 | 0 | 0 | 0 | 0 | 0 | 0 | 100 |
| HEART2005610 | 0 | 0 | 0 | 0 | 0 | 0 | 100 |
| HEART2006131 | 0 | 0 | 0 | 0 | 0 | 0 | 100 |
| HEART2006195 | 0 | 0 | 0 | 0 | 0 | 0 | 63.227 |
| HEART2006310 | 0 | 0 | 0 | 0 | 0 | 0 | 100 |
| HEART2006334 | 0 | 0 | 0 | 0 | 0 | 0 | 100 |
| HEART2006487 | 0 | 0 | 0 | 0 | 0 | 0 | 64.867 |
| HEART2006521 | 0 | 0 | 0 | 0 | 0 | 0 | 15.956 |
| HEART2006557 | 0 | 0 | 0 | 0 | 0 | 0 | 100 |
| HEART2006787 | 0 | 0 | 0 | 0 | 0 | 0 | 100 |
| HEART2006789 | 0 | 0 | 0 | 0 | 0 | 0 | 100 |
| HEART2006909 | 0 | 0 | 0 | 0 | 0 | 0 | 100 |
| HEART2007031 | 0 | 0 | 0 | 0 | 0 | 0 | 75.806 |
| HEART2007231 | 0 | 0 | 0 | 0 | 0 | 0 | 32.316 |
| HEART2007443 | 0 | 0 | 0 | 0 | 0 | 0 | 100 |
| HEART2007696 | 0 | 0 | 0 | 0 | 0 | 0 | 33.229 |
| HEART2007767 | 0 | 0 | 0 | 0 | 0 | 0 | 100 |
| HEART2008108 | 0 | 0 | 0 | 0 | 0 | 0 | 100 |
| HEART2008111 | 0 | 0 | 0 | 0 | 0 | 0 | 100 |
| HEART2008257 | 0 | 0 | 0 | 0 | 0 | 0 | 100 |
| HEART2008509 | 0 | 0 | 0 | 0 | 0 | 0 | 78.725 |
| HEART2008994 | 0 | 0 | 0 | 0 | 0 | 0 | 100 |
| HEART2009000 | 0 | 0 | 0 | 0 | 0 | 0 | 68.952 |
| HEART2009599 | 0 | 0 | 0 | 0 | 0 | 0 | 100 |
| HEART2009680 | 0 | 0 | 0 | 0 | 0 | 0 | 100 |
| HEART2010391 | 0 | 0 | 0 | 0 | 0 | 0 | 100 |
| HEART2010492 | 0 | 0 | 0 | 0 | 0 | 0 | 100 |
| HEART2010495 | 0 | 0 | 0 | 0 | 0 | 0 | 49.759 |
| HHDPC1000083 | 0 | 0 | 0 | 0 | 6.499 | 0 | 0 |
| HHDPC1000114 | 15.131 | 0 | 0 | 0 | 4.824 | 0 | 0 |
| HHDPC1000163 | 0 | 0 | 0 | 0 | 8.349 | 0 | 8.389 |
| HHDPC2000115 | 0 | 0 | 0 | 0 | 0 | 0 | 17.452 |
| HHDPC2003381 | 0 | 75.443 | 0 | 0 | 0 | 0 | 0 |
| HHDPC2003439 | 0 | 0 | 0 | 0 | 0 | 1.609 | 0 |
| HHDPC2003983 | 0 | 12.047 | 0 | 0 | 3.698 | 0 | 0 |
| HHDPC2008297 | 0 | 0 | 0 | 0 | 0 | 47.331 | 0 |
| HLUNG1000055 | 0 | 11.709 | 0 | 11.642 | 0 | 0 | 3.611 |
| HLUNG2000063 | 0 | 0 | 0 | 0 | 0 | 15.606 | 16.454 |
| HLUNG2000501 | 0 | 0 | 0 | 18.449 | 0 | 0 | 5.723 |
| HLUNG2001712 | 0 | 0 | 0 | 0 | 0 | 0 | 21.996 |
| HLUNG2002465 | 0 | 0 | 0 | 26.011 | 0 | 0 | 0 |
| HLUNG2003331 | 0 | 0 | 0 | 0 | 0 | 0 | 55.232 |
| HLUNG2004534 | 0 | 62.747 | 0 | 0 | 0 | 0 | 0 |
| HLUNG2007210 | 0 | 0 | 0 | 0 | 0 | 13.007 | 0 |
| HLUNG2007245 | 0 | 75.147 | 0 | 0 | 0 | 0 | 0 |
| HSYRA1000062 | 0 | 0 | 0 | 9.212 | 2.844 | 5.421 | 0 |
| HSYRA1000119 | 0 | 0 | 0 | 0 | 3.053 | 2.909 | 0 |
| HSYRA2000137 | 0 | 0 | 0 | 0 | 0 | 14.342 | 15.121 |
| HSYRA2000159 | 0 | 0 | 0 | 0 | 0 | 0 | 1.609 |
| HSYRA2000224 | 0 | 38.896 | 0 | 0 | 0 | 0 | 0 |
| HSYRA2000232 | 0 | 0 | 0 | 15.903 | 0 | 0 | 0 |
| HSYRA2000248 | 14.153 | 0 | 0 | 0 | 0 | 8.601 | 0 |
| HSYRA2000255 | 0 | 0 | 0 | 0 | 0 | 25.553 | 0 |
| HSYRA2000332 | 16.242 | 0 | 0 | 16.775 | 0 | 9.871 | 0 |
| HSYRA2000743 | 0 | 0 | 0 | 0 | 0 | 0 | 5.163 |
| HSYRA2000792 | 0 | 0 | 0 | 0 | 0 | 15.324 | 0 |
| HSYRA2001103 | 0 | 0 | 0 | 0 | 5.631 | 0 | 0 |
| HSYRA2001138 | 0 | 5.515 | 0 | 0 | 0 | 0 | 0 |
| HSYRA2001232 | 0 | 0 | 0 | 0 | 0 | 24.478 | 0 |
| HSYRA2001476 | 0 | 0 | 0 | 0 | 6.411 | 12.219 | 0 |
| HSYRA2001567 | 0 | 0 | 0 | 0 | 0 | 28.361 | 0 |
| HSYRA2001574 | 0 | 0 | 0 | 0 | 4.996 | 0 | 0 |
| HSYRA2001615 | 0 | 0 | 0 | 0 | 0 | 13.123 | 0 |
| HSYRA2001621 | 0 | 0 | 0 | 0 | 2.437 | 0 | 0 |
| HSYRA2005628 | 0 | 0 | 0 | 0 | 0 | 42.436 | 0 |
| IMR321000158 | 0 | 0 | 0 | 0 | 5.054 | 0 | 0 |
| IMR322000072 | 0 | 0 | 0 | 0 | 0 | 5.077 | 0 |
| IMR322000178 | 38.729 | 0 | 0 | 39.999 | 0 | 0 | 0 |
| IMR322000223 | 0 | 0 | 0 | 0 | 0 | 4.308 | 4.542 |
| IMR322000374 | 0 | 0 | 0 | 0 | 36.572 | 0 | 0 |
| IMR322000555 | 0 | 0 | 0 | 0 | 0 | 0 | 39.800 |
| IMR322000730 | 2.854 | 0 | 0 | 8.843 | 0 | 0 | 2.743 |
| IMR322000742 | 0 | 0 | 0 | 35.613 | 0 | 0 | 0 |
| IMR322000791 | 0 | 0 | 0 | 0 | 0 | 32.829 | 0 |
| IMR322001317 | 0 | 0 | 0 | 0 | 0 | 0 | 10.536 |
| IMR322001435 | 0 | 0 | 0 | 0 | 0 | 11.889 | 0 |
| IMR322001534 | 0 | 0 | 0 | 0 | 0 | 0 | 55.124 |
| IMR322001600 | 3.353 | 0 | 0 | 0 | 2.138 | 3.057 | 0 |
| IMR322001710 | 0 | 0 | 0 | 0 | 12.877 | 0 | 0 |
| KIDNE1000008 | 0 | 43.681 | 0 | 0 | 0 | 0 | 0 |
| KIDNE1000143 | 0 | 43.153 | 0 | 0 | 0 | 0 | 0 |
| KIDNE2000085 | 0 | 0 | 0 | 0 | 0 | 6.507 | 0 |
| KIDNE2000315 | 0 | 0 | 0 | 0 | 0 | 0 | 45.557 |
| KIDNE2000403 | 0 | 17.871 | 0 | 0 | 0 | 0 | 0 |
| KIDNE2000497 | 0 | 0 | 0 | 0 | 0 | 0 | 20.919 |
| KIDNE2000665 | 0 | 41.875 | 0 | 0 | 0 | 0 | 12.914 |
| KIDNE2000832 | 0 | 67.408 | 0 | 0 | 0 | 0 | 0 |
| KIDNE2000909 | 0 | 0 | 0 | 44.154 | 0 | 0 | 0 |
| KIDNE2001467 | 0 | 0 | 0 | 0 | 3.461 | 3.299 | 0 |
| KIDNE2002839 | 0 | 0 | 0 | 0 | 0 | 0 | 48.974 |
| KIDNE2002872 | 0 | 12.368 | 0 | 0 | 0 | 0 | 0 |
| KIDNE2002883 | 0 | 0 | 0 | 0 | 0 | 0 | 15.131 |
| KIDNE2002980 | 0 | 0 | 0 | 67.783 | 0 | 0 | 0 |
| KIDNE2003507 | 70.503 | 0 | 0 | 0 | 0 | 0 | 0 |
| KIDNE2005676 | 0 | 0 | 0 | 0 | 0 | 8.902 | 0 |
| KIDNE2006062 | 0 | 60.803 | 0 | 0 | 0 | 0 | 0 |
| KIDNE2006248 | 6.474 | 0 | 0 | 0 | 0 | 0 | 0 |
| KIDNE2008048 | 0 | 0 | 0 | 0 | 3.562 | 6.789 | 0 |
| KIDNE2010265 | 0 | 0 | 0 | 0 | 15.686 | 0 | 0 |
| KIDNE2014290 | 0 | 0 | 0 | 0 | 4.655 | 0 | 0 |
| KIDNE2017040 | 0 | 0 | 0 | 0 | 0 | 21.278 | 0 |
| LIVER2005218 | 51.359 | 0 | 0 | 0 | 0 | 0 | 0 |
| LIVER2007783 | 0 | 44.385 | 0 | 0 | 0 | 0 | 0 |
| MAMGL1000032 | 0 | 0 | 0 | 6.931 | 1.070 | 0 | 0 |
| MAMGL1000035 | 0 | 0 | 0 | 0 | 0 | 0 | 1.343 |
| MAMGL1000173 | 0 | 0 | 0 | 0 | 0 | 0 | 1.768 |
| MESAN1000080 | 0 | 30.885 | 0 | 0 | 0 | 9.034 | 0 |
| MESAN1000126 | 0 | 0 | 0 | 0 | 0 | 11.299 | 0 |
| MESAN2000267 | 0 | 0 | 0 | 0 | 2.719 | 0 | 0 |
| MESAN2000572 | 0 | 22.116 | 0 | 0 | 0 | 0 | 0 |
| MESAN2000608 | 0 | 17.010 | 0 | 0 | 0 | 4.976 | 5.246 |
| MESAN2000909 | 0 | 0 | 0 | 0 | 0 | 34.948 | 0 |
| MESAN2002086 | 0 | 0 | 0 | 0 | 8.292 | 10.536 | 0 |
| MESAN2002790 | 0 | 0 | 0 | 0 | 16.238 | 0 | 0 |
| MESAN2003622 | 0 | 0 | 0 | 12.925 | 3.990 | 0 | 0 |
| MESAN2005766 | 0 | 0 | 0 | 0 | 0 | 6.240 | 6.579 |
| MESAN2005958 | 0 | 0 | 0 | 0 | 0 | 17.040 | 0 |
| MESAN2009580 | 0 | 24.023 | 0 | 0 | 0 | 0 | 0 |
| MESAN2012735 | 0 | 0 | 0 | 0 | 0 | 63.051 | 0 |
| MESAN2013908 | 0 | 0 | 0 | 0 | 0 | 43.658 | 0 |
| NB9N41000121 | 0 | 0 | 0 | 0 | 0 | 4.752 | 0 |
| NB9N41000340 | 0 | 0 | 0 | 0 | 9.629 | 0 | 9.675 |
| NB9N42000104 | 0 | 0 | 0 | 0 | 0 | 5.245 | 0 |
| NB9N42000342 | 0 | 0 | 0 | 0 | 14.361 | 0 | 0 |
| NHNPC2000187 | 0 | 0 | 0 | 0 | 0 | 0 | 3.019 |
| NHNPC2000206 | 0 | 0 | 0 | 0 | 0 | 4.345 | 0 |
| NHNPC2001931 | 0 | 0 | 0 | 0 | 0 | 6.040 | 3.184 |
| NT2NE1000059 | 0 | 0 | 0 | 0 | 0 | 34.225 | 0 |
| NT2NE2000056 | 0 | 0 | 0 | 8.449 | 0 | 0 | 0 |
| NT2NE2000174 | 15.892 | 0 | 0 | 0 | 0 | 0 | 0 |
| NT2NE2000260 | 0 | 2.613 | 0 | 2.598 | 0 | 0 | 0 |
| NT2NE2000383 | 0 | 0 | 0 | 0 | 10.089 | 0 | 0 |
| NT2NE2000575 | 5.006 | 0 | 0 | 0 | 3.192 | 1.521 | 1.604 |
| NT2NE2000636 | 0 | 0 | 0 | 0 | 23.974 | 0 | 0 |
| NT2NE2000707 | 0 | 0 | 0 | 0 | 0 | 0 | 4.465 |
| NT2NE2000963 | 9.554 | 4.962 | 0 | 0 | 0 | 1.452 | 0 |
| NT2NE2001000 | 0 | 0 | 0 | 0 | 0 | 0 | 5.741 |
| NT2NE2001655 | 0 | 0 | 0 | 0 | 0 | 0 | 18.680 |
| NT2NE2002620 | 0 | 0 | 0 | 0 | 6.351 | 0 | 0 |
| NT2NE2003569 | 0 | 37.541 | 0 | 0 | 0 | 0 | 0 |
| NT2NE2003921 | 0 | 0 | 0 | 0 | 0 | 0 | 40.699 |
| NT2NE2005688 | 0 | 0 | 0 | 0 | 4.958 | 0 | 0 |
| NT2NE2006458 | 0 | 0 | 0 | 30.400 | 0 | 8.944 | 0 |
| NT2NE2008077 | 0 | 0 | 0 | 0 | 64.689 | 0 | 0 |
| NT2NE2018180 | 0 | 0 | 0 | 0 | 24.965 | 0 | 0 |
| NT2NE2019091 | 0 | 0 | 0 | 0 | 12.569 | 0 | 0 |
| NT2RI1000027 | 0 | 0 | 0 | 21.745 | 0 | 0 | 0 |
| NT2RI1000127 | 0 | 0 | 0 | 39.035 | 0 | 0 | 0 |
| NT2RI2000064 | 0 | 0 | 0 | 0 | 0 | 0 | 3.591 |
| NT2RI2000282 | 0 | 6.847 | 0 | 0 | 0 | 4.006 | 2.112 |
| NT2RI2000284 | 0 | 0 | 0 | 9.804 | 0 | 0 | 3.041 |
| NT2RI2000669 | 0 | 0 | 0 | 0 | 0 | 77.688 | 0 |
| NT2RI2000671 | 0 | 0 | 0 | 0 | 0 | 0 | 7.017 |
| NT2RI2000727 | 0 | 0 | 0 | 0 | 5.153 | 0 | 0 |
| NT2RI2000974 | 0 | 0 | 0 | 0 | 0 | 13.649 | 0 |
| NT2RI2001540 | 0 | 0 | 0 | 2.449 | 1.134 | 0 | 1.139 |
| NT2RI2001657 | 0 | 0 | 0 | 0 | 3.552 | 3.386 | 0 |
| NT2RI2001731 | 39.438 | 0 | 0 | 0 | 0 | 0 | 0 |
| NT2RI2001859 | 0 | 0 | 0 | 0 | 0 | 6.140 | 0 |
| NT2RI2002091 | 0 | 0 | 0 | 0.577 | 2.939 | 5.601 | 1.253 |
| NT2RI2002391 | 0 | 0 | 0 | 34.882 | 0 | 0 | 0 |
| NT2RI2002654 | 0 | 12.708 | 0 | 12.634 | 7.801 | 0 | 3.919 |
| NT2RI2003304 | 5.465 | 5.677 | 0 | 0 | 0 | 1.660 | 0 |
| NT2RI2003338 | 0 | 21.903 | 0 | 0 | 6.723 | 6.407 | 0 |
| NT2RI2003407 | 0 | 0 | 0 | 0 | 8.036 | 7.658 | 0 |
| NT2RI2003420 | 0 | 0 | 0 | 0 | 0 | 0 | 10.273 |
| NT2RI2003481 | 0 | 0 | 0 | 0 | 12.821 | 0 | 0 |
| NT2RI2003695 | 0 | 0 | 0 | 0 | 5.228 | 4.983 | 0 |
| NT2RI2003738 | 0 | 0 | 0 | 0 | 0 | 1.107 | 2.334 |
| NT2RI2003751 | 0 | 29.966 | 0 | 0 | 18.395 | 0 | 9.242 |
| NT2RI2003921 | 0 | 0 | 0 | 10.200 | 0 | 3.001 | 0 |
| NT2RI2003993 | 0 | 0 | 0 | 0 | 0 | 0 | 25.326 |
| NT2RI2004059 | 0 | 0 | 0 | 0 | 0 | 0 | 45.459 |
| NT2RI2004535 | 0 | 0 | 0 | 0 | 12.709 | 0 | 0 |
| NT2RI2005087 | 0 | 0 | 0 | 92.209 | 0 | 0 | 0 |
| NT2RI2005150 | 0 | 0 | 0 | 0 | 4.575 | 0 | 0 |
| NT2RI2005335 | 0 | 0 | 0 | 8.325 | 0 | 0 | 0 |
| NT2RI2005405 | 0 | 0 | 0 | 0 | 3.728 | 3.553 | 0 |
| NT2RI2005723 | 0 | 0 | 0 | 0 | 0 | 0 | 16.739 |
| NT2RI2005851 | 0 | 0 | 0 | 0 | 0 | 23.580 | 0 |
| NT2RI2006412 | 0 | 0 | 0 | 0 | 0 | 0 | 21.874 |
| NT2RI2006825 | 0 | 0 | 0 | 0 | 0 | 17.348 | 0 |
| NT2RI2006856 | 0 | 0 | 0 | 0 | 0 | 0 | 3.125 |
| NT2RI2007054 | 0 | 0 | 0 | 0 | 0 | 0 | 8.661 |
| NT2RI2007133 | 0 | 0 | 0 | 0 | 2.605 | 0 | 2.617 |
| NT2RI2007148 | 0 | 0 | 0 | 0 | 6.270 | 0 | 6.300 |
| NT2RI2007384 | 0 | 0 | 0 | 0 | 0 | 0 | 2.889 |
| NT2RI2007754 | 0 | 0 | 0 | 0 | 0 | 0 | 64.734 |
| NT2RI2007987 | 0 | 0 | 0 | 0 | 0 | 11.393 | 0 |
| NT2RI2008045 | 0 | 0 | 0 | 0 | 5.155 | 4.913 | 0 |
| NT2RI2008141 | 0 | 0 | 0 | 0 | 14.197 | 0 | 0 |
| NT2RI2008171 | 0 | 0 | 0 | 0 | 0 | 0 | 29.622 |
| NT2RI2008396 | 9.179 | 0 | 0 | 0 | 0 | 2.789 | 0 |
| NT2RI2008481 | 0 | 0 | 0 | 0 | 0 | 0 | 4.934 |
| NT2RI2008526 | 0 | 0 | 0 | 0 | 5.090 | 0 | 0 |
| NT2RI2008598 | 0 | 0 | 0 | 0 | 55.694 | 0 | 0 |
| NT2RI2008622 | 0 | 0 | 0 | 0 | 20.953 | 0 | 0 |
| NT2RI2008791 | 0 | 0 | 0 | 0 | 0 | 0 | 7.784 |
| NT2RI2009037 | 0 | 0 | 0 | 0 | 5.597 | 0 | 0 |
| NT2RI2009065 | 0 | 0 | 0 | 10.045 | 3.101 | 0 | 0 |
| NT2RI2009194 | 0 | 0 | 0 | 0 | 6.525 | 0 | 0 |
| NT2RI2009233 | 0 | 0 | 0 | 0 | 14.976 | 0 | 0 |
| NT2RI2009269 | 0 | 0 | 0 | 0 | 0 | 0 | 6.593 |
| NT2RI2009583 | 0 | 4.840 | 0 | 0 | 0 | 0 | 0 |
| NT2RI2022468 | 0 | 0 | 0 | 0 | 44.507 | 0 | 0 |
| NT2RI3007543 | 0 | 0 | 0 | 18.247 | 5.633 | 10.737 | 0 |
| NT2RP6000033 | 0 | 89.975 | 0 | 0 | 0 | 0 | 0 |
| NT2RP6000039 | 0 | 0 | 0 | 38.566 | 0 | 0 | 0 |
| NT2RP6000123 | 0 | 0 | 0 | 8.419 | 0 | 0 | 0 |
| NT2RP7000076 | 0 | 0 | 0 | 0 | 0 | 0 | 4.066 |
| NT2RP7001166 | 0 | 0 | 0 | 0 | 0 | 4.732 | 0 |
| NT2RP7001283 | 0 | 12.176 | 0 | 0 | 3.737 | 3.562 | 0 |
| NT2RP7001591 | 0 | 56.239 | 0 | 0 | 0 | 0 | 0 |
| NT2RP7001962 | 0 | 0 | 0 | 0 | 17.637 | 0 | 0 |
| NT2RP7002028 | 0 | 43.470 | 0 | 0 | 0 | 0 | 0 |
| NT2RP7002379 | 0 | 0 | 0 | 0 | 3.342 | 3.185 | 0 |
| NT2RP7002449 | 0 | 0 | 0 | 0 | 0 | 12.783 | 0 |
| NT2RP7002450 | 0 | 0 | 0 | 0 | 0 | 0 | 17.805 |
| NT2RP7002619 | 0 | 0 | 0 | 0 | 0 | 6.894 | 0 |
| NT2RP7003960 | 0 | 0 | 0 | 0 | 0 | 10.484 | 0 |
| NT2RP7004348 | 0 | 0 | 0 | 0 | 0 | 14.300 | 0 |
| NT2RP7004751 | 0 | 0 | 0 | 0 | 0 | 2.962 | 0 |
| NT2RP7004925 | 0 | 0 | 0 | 0 | 8.383 | 0 | 0 |
| NT2RP7006075 | 0 | 0 | 0 | 0 | 0 | 16.578 | 0 |
| NT2RP7006717 | 0 | 0 | 0 | 0 | 0 | 0 | 4.389 |
| NT2RP7007154 | 0 | 0 | 0 | 0 | 0 | 3.608 | 0 |
| NT2RP7007177 | 0 | 0 | 0 | 0 | 9.042 | 0 | 0 |
| NT2RP7008623 | 89.627 | 0 | 0 | 0 | 0 | 0 | 0 |
| NT2RP7009087 | 0 | 0 | 0 | 0 | 16.129 | 0 | 0 |
| NT2RP7009149 | 0 | 0 | 0 | 0 | 0 | 30.278 | 0 |
| NT2RP7012776 | 0 | 0 | 0 | 0 | 55.802 | 0 | 0 |
| NT2RP7014005 | 0 | 0 | 0 | 0 | 0 | 36.812 | 0 |
| NT2RP8007920 | 0 | 0 | 0 | 0 | 0 | 12.517 | 0 |
| NTONG1000182 | 0 | 0 | 0 | 0 | 0 | 0 | 8.259 |
| NTONG1000257 | 0 | 0 | 0 | 0 | 0 | 6.963 | 0 |
| NTONG2000265 | 0 | 0 | 0 | 0 | 5.660 | 0 | 0 |
| NTONG2000878 | 24.591 | 0 | 0 | 0 | 0 | 7.472 | 0 |
| NTONG2001567 | 0 | 0 | 0 | 6.595 | 2.036 | 0 | 0 |
| NTONG2001762 | 0 | 0 | 0 | 0 | 11.973 | 0 | 0 |
| NTONG2002948 | 0 | 0 | 0 | 0 | 0 | 0 | 44.440 |
| NTONG2003839 | 0 | 0 | 0 | 0 | 17.408 | 0 | 0 |
| NTONG2007658 | 0 | 0 | 0 | 43.039 | 0 | 0 | 0 |
| NTONG2008483 | 0 | 0 | 0 | 0 | 0 | 0 | 5.330 |
| OCBBF1000086 | 0 | 0 | 0 | 0 | 0 | 4.843 | 0 |
| OCBBF1000175 | 0 | 0 | 0 | 0 | 0 | 0 | 48.053 |
| OCBBF2000712 | 0 | 0 | 0 | 0 | 0 | 5.980 | 0 |
| OCBBF2000982 | 0 | 18.864 | 0 | 0 | 0 | 0 | 0 |
| OCBBF2001492 | 0 | 0 | 0 | 0 | 0 | 0 | 4.577 |
| OCBBF2002626 | 0 | 0 | 0 | 0 | 0 | 5.343 | 0 |
| OCBBF2004273 | 0 | 19.389 | 0 | 0 | 5.951 | 0 | 0 |
| OCBBF2007829 | 0 | 0 | 0 | 0 | 0 | 3.474 | 3.663 |
| OCBBF2008144 | 0 | 62.383 | 0 | 0 | 0 | 0 | 0 |
| OCBBF2008483 | 0 | 0 | 0 | 0 | 57.797 | 0 | 0 |
| OCBBF2009424 | 0 | 0 | 0 | 0 | 0 | 45.919 | 0 |
| OCBBF2010830 | 0 | 0 | 0 | 0 | 0 | 11.135 | 0 |
| OCBBF2011021 | 0 | 0 | 0 | 0 | 9.597 | 0 | 0 |
| OCBBF2011536 | 0 | 0 | 0 | 42.905 | 0 | 12.623 | 0 |
| OCBBF2012714 | 0 | 0 | 0 | 0 | 38.538 | 0 | 0 |
| OCBBF2014292 | 0 | 0 | 0 | 0 | 6.876 | 0 | 0 |
| OCBBF2014576 | 0 | 0 | 0 | 78.585 | 0 | 0 | 0 |
| OCBBF2018707 | 0 | 0 | 0 | 0 | 14.932 | 0 | 0 |
| OCBBF2018956 | 35.243 | 0 | 0 | 0 | 0 | 0 | 0 |
| OCBBF2024850 | 0 | 0 | 0 | 0 | 0 | 0 | 14.826 |
| OCBBF2035226 | 0 | 12.708 | 0 | 0 | 0 | 0 | 0 |
| PEBLM2006366 | 0 | 0 | 0 | 0 | 0 | 0 | 18.464 |
| PEBLM2006709 | 0 | 0 | 0 | 0 | 0 | 35.204 | 0 |
| PEBLM2007774 | 0 | 6.515 | 0 | 0 | 0 | 0 | 2.009 |
| PLACE5000080 | 0 | 0 | 0 | 0 | 0 | 78.048 | 0 |
| PLACE5000170 | 0 | 0 | 0 | 0 | 0 | 0 | 23.195 |
| PLACE5000282 | 0 | 0 | 0 | 0 | 0 | 78.048 | 0 |
| PLACE6000325 | 0 | 0 | 0 | 0 | 0 | 51.259 | 0 |
| PLACE6000379 | 3.678 | 0 | 0 | 0 | 1.173 | 4.470 | 0 |
| PLACE6000414 | 0 | 0 | 0 | 0 | 0 | 24.324 | 0 |
| PLACE6001064 | 0 | 0 | 0 | 0 | 0 | 7.260 | 0 |
| PLACE6001281 | 0 | 0 | 0 | 0 | 0 | 0 | 22.967 |
| PLACE6002084 | 0 | 0 | 0 | 0 | 2.452 | 0 | 0 |
| PLACE6003383 | 0 | 0 | 0 | 0 | 0 | 0 | 15.925 |
| PLACE6004312 | 12.559 | 0 | 0 | 0 | 0 | 0 | 4.023 |
| PLACE6004578 | 0 | 26.814 | 0 | 0 | 0 | 0 | 0 |
| PLACE6006042 | 0 | 0 | 0 | 0 | 0 | 34.446 | 0 |
| PLACE6006186 | 0 | 0 | 0 | 0 | 0 | 45.767 | 0 |
| PLACE6007436 | 0 | 0 | 0 | 0 | 0 | 0 | 23.968 |
| PLACE6010484 | 0 | 0 | 0 | 0 | 0 | 9.780 | 0 |
| PLACE6010848 | 0 | 0 | 0 | 0 | 54.330 | 0 | 0 |
| PLACE6011041 | 0 | 58.854 | 0 | 0 | 0 | 0 | 0 |
| PLACE6012028 | 0 | 0 | 0 | 0 | 0 | 0 | 41.878 |
| PLACE6014882 | 0 | 0 | 0 | 0 | 78.861 | 0 | 0 |
| PLACE6015051 | 0 | 0 | 0 | 0 | 0 | 0 | 30.753 |
| PLACE6016383 | 0 | 0 | 0 | 0 | 0 | 0 | 43.129 |
| PLACE6017578 | 0 | 0 | 0 | 0 | 0 | 63.999 | 0 |
| PLACE6017791 | 0 | 0 | 0 | 0 | 0 | 0 | 73.274 |
| PLACE6018487 | 0 | 0 | 0 | 28.705 | 0 | 0 | 0 |
| PLACE6019385 | 0 | 87.742 | 0 | 0 | 0 | 0 | 0 |
| PLACE6020145 | 0 | 0 | 0 | 0 | 0 | 0 | 55.988 |
| PROST1000097 | 0 | 0 | 0 | 12.646 | 0 | 0 | 0 |
| PROST1000199 | 0 | 0 | 0 | 0 | 0 | 4.623 | 4.874 |
| PROST1000343 | 59.153 | 0 | 0 | 0 | 0 | 0 | 0 |
| PROST1000512 | 0 | 46.564 | 0 | 0 | 0 | 0 | 0 |
| PROST1000536 | 0 | 0 | 0 | 0 | 39.920 | 0 | 0 |
| PROST1000564 | 0 | 29.078 | 0 | 0 | 0 | 8.506 | 0 |
| PROST2000138 | 0 | 1.875 | 0 | 0 | 1.151 | 2.742 | 1.156 |
| PROST2001283 | 0 | 0 | 0 | 45.762 | 0 | 0 | 14.195 |
| PROST2002078 | 0 | 0 | 0 | 0 | 0 | 0 | 16.213 |
| PROST2003338 | 0 | 0 | 0 | 0 | 0 | 7.082 | 7.467 |
| PROST2003517 | 0 | 0 | 0 | 0 | 0 | 0 | 17.563 |
| PROST2004095 | 0 | 10.125 | 0 | 0 | 3.108 | 0 | 3.122 |
| PROST2004416 | 0 | 0 | 0 | 0 | 4.515 | 0 | 0 |
| PROST2008489 | 0 | 0 | 0 | 0 | 0 | 0 | 4.321 |
| PROST2010782 | 0 | 0 | 78.029 | 0 | 0 | 0 | 0 |
| PROST2012745 | 0 | 0 | 0 | 0 | 0 | 0 | 25.800 |
| PROST2012780 | 0 | 0 | 0 | 0 | 0 | 0 | 48.531 |
| PROST2013053 | 0 | 64.069 | 0 | 0 | 0 | 0 | 0 |
| PROST2017203 | 27.042 | 0 | 0 | 27.929 | 0 | 0 | 0 |
| PROST2017692 | 0 | 0 | 0 | 0 | 0 | 13.988 | 0 |
| PUAEN2000374 | 0 | 0 | 0 | 0 | 2.125 | 0 | 0 |
| PUAEN2000497 | 53.684 | 0 | 0 | 0 | 0 | 0 | 0 |
| SALGL1000157 | 0 | 0 | 0 | 0 | 0.863 | 0 | 0.868 |
| SKMUS1000064 | 0 | 0 | 0 | 0 | 0 | 0 | 46.964 |
| SKMUS1000084 | 0 | 0 | 0 | 0 | 10.225 | 0 | 0 |
| SKMUS1000138 | 0 | 0 | 0 | 0 | 0 | 0 | 5.766 |
| SKMUS1000186 | 0 | 0 | 0 | 0 | 0 | 2.540 | 0 |
| SKMUS2000198 | 0 | 0 | 0 | 0 | 0 | 0 | 21.074 |
| SKMUS2000361 | 0 | 0 | 0 | 0 | 0 | 0 | 7.134 |
| SKMUS2000380 | 0 | 0 | 0 | 0 | 0 | 13.722 | 14.467 |
| SKMUS2000458 | 0 | 0 | 0 | 0 | 0 | 0 | 12.752 |
| SKMUS2000679 | 13.951 | 0 | 0 | 0 | 0 | 0 | 4.469 |
| SKMUS2000757 | 0 | 0 | 0 | 0 | 32.810 | 0 | 0 |
| SKMUS2000821 | 0 | 0 | 0 | 0 | 0 | 0 | 5.295 |
| SKMUS2000873 | 0 | 0 | 0 | 0 | 0 | 0 | 58.714 |
| SKMUS2000931 | 0 | 0 | 0 | 0 | 0 | 0 | 3.630 |
| SKMUS2001008 | 0 | 0 | 0 | 0 | 0 | 0 | 6.322 |
| SKMUS2001501 | 0 | 0 | 0 | 0 | 0 | 0 | 29.082 |
| SKMUS2001634 | 0 | 28.321 | 0 | 0 | 0 | 8.284 | 0 |
| SKMUS2006394 | 0 | 0 | 0 | 0 | 0 | 0 | 32.130 |
| SKNMC1000137 | 0 | 0 | 0 | 22.798 | 7.038 | 0 | 0 |
| SKNMC1000168 | 0 | 0 | 0 | 18.010 | 0 | 0 | 0 |
| SKNMC2000224 | 0 | 0 | 0 | 0 | 0 | 39.635 | 0 |
| SKNMC2000635 | 0 | 3.880 | 0 | 0 | 7.146 | 6.810 | 0 |
| SKNMC2000649 | 0 | 0 | 0 | 0 | 0 | 11.546 | 0 |
| SKNMC2000698 | 0 | 0 | 0 | 0 | 0 | 13.070 | 0 |
| SKNSH2000716 | 0 | 37.491 | 0 | 0 | 0 | 0 | 0 |
| SKNSH2001931 | 0 | 0 | 0 | 0 | 33.930 | 0 | 0 |
| SKNSH2002768 | 0 | 0 | 0 | 0 | 0 | 0 | 15.796 |
| SKNSH2006234 | 0 | 0 | 0 | 0 | 23.241 | 0 | 0 |
| SMINT1000039 | 0 | 0 | 0 | 0 | 65.296 | 0 | 0 |
| SMINT1000131 | 0 | 0 | 0 | 0 | 6.080 | 5.795 | 0 |
| SMINT1000192 | 0 | 0 | 0 | 64.044 | 0 | 0 | 0 |
| SMINT2000277 | 0 | 0 | 0 | 0 | 0 | 0 | 12.095 |
| SMINT2000396 | 0 | 0 | 0 | 0 | 8.127 | 0 | 0 |
| SMINT2000441 | 0 | 0 | 0 | 0 | 2.997 | 2.856 | 0 |
| SMINT2000468 | 0 | 0 | 0 | 0 | 0 | 14.477 | 0 |
| SMINT2000694 | 0 | 0 | 0 | 0 | 0 | 0 | 7.930 |
| SMINT2001818 | 0 | 0 | 0 | 0 | 0 | 0 | 6.254 |
| SMINT2002689 | 0 | 0 | 0 | 0 | 0 | 0 | 7.842 |
| SMINT2003074 | 0 | 0 | 0 | 0 | 9.101 | 0 | 0 |
| SMINT2003169 | 0 | 0 | 0 | 0 | 3.123 | 0 | 3.138 |
| SMINT2003905 | 0 | 0 | 0 | 0 | 0 | 4.899 | 0 |
| SMINT2004144 | 0 | 9.818 | 0 | 0 | 0 | 0 | 0 |
| SMINT2007433 | 0 | 0 | 0 | 0 | 0 | 0 | 55.688 |
| SMINT2012122 | 0 | 0 | 0 | 0 | 0 | 0 | 23.322 |
| SMINT2012291 | 0 | 0 | 0 | 0 | 0 | 0 | 30.763 |
| SMINT2013031 | 64.629 | 0 | 0 | 0 | 0 | 0 | 0 |
| SMINT2017599 | 85.510 | 0 | 0 | 0 | 0 | 0 | 0 |
| SMINT2018353 | 0 | 0 | 0 | 0 | 0 | 0 | 65.405 |
| SPLEN1000116 | 0 | 0 | 0 | 0 | 0 | 0 | 3.302 |
| SPLEN1000166 | 0 | 0 | 0 | 0 | 0 | 7.153 | 0 |
| SPLEN2000258 | 0 | 0 | 0 | 0 | 29.066 | 0 | 0 |
| SPLEN2000348 | 0 | 0 | 0 | 0 | 0 | 17.701 | 0 |
| SPLEN2000874 | 0 | 0 | 0 | 10.483 | 0 | 0 | 3.252 |
| SPLEN2000882 | 0 | 0 | 0 | 0 | 11.663 | 0 | 0 |
| SPLEN2002477 | 33.838 | 0 | 0 | 0 | 0 | 0 | 0 |
| SPLEN2002744 | 0 | 0 | 0 | 0 | 0 | 0 | 14.879 |
| SPLEN2005806 | 0 | 13.228 | 0 | 0 | 0 | 3.869 | 0 |
| SPLEN2005927 | 0 | 0 | 0 | 0 | 0 | 0 | 28.728 |
| SPLEN2006325 | 0 | 0 | 0 | 0 | 55.314 | 0 | 0 |
| SPLEN2013503 | 0 | 0 | 0 | 53.981 | 0 | 0 | 0 |
| SPLEN2013690 | 0 | 0 | 0 | 0 | 0 | 68.333 | 0 |
| SPLEN2014572 | 0 | 0 | 0 | 0 | 0 | 39.379 | 0 |
| SPLEN2016268 | 0 | 0 | 0 | 9.292 | 0 | 2.734 | 2.882 |
| SPLEN2016421 | 0 | 0 | 0 | 0 | 0 | 13.109 | 13.821 |
| SPLEN2016743 | 0 | 0 | 0 | 0 | 0 | 0 | 38.250 |
| SPLEN2016972 | 0 | 43.864 | 0 | 0 | 0 | 0 | 0 |
| SPLEN2018302 | 0 | 0 | 0 | 0 | 40.372 | 0 | 0 |
| SPLEN2019405 | 0 | 30.978 | 0 | 0 | 0 | 0 | 0 |
| SPLEN2029912 | 0 | 0 | 0 | 0 | 10.846 | 0 | 0 |
| SPLEN2032157 | 0 | 0 | 0 | 0 | 37.939 | 0 | 0 |
| STOMA1000189 | 0 | 0 | 0 | 33.633 | 0 | 0 | 0 |
| STOMA2000121 | 0 | 26.360 | 0 | 0 | 0 | 0 | 0 |
| STOMA2000395 | 0 | 0 | 0 | 0 | 14.173 | 0 | 0 |
| STOMA2000396 | 0 | 0 | 0 | 0 | 0 | 0 | 34.381 |
| STOMA2002166 | 12.183 | 0 | 0 | 0 | 3.884 | 0 | 0 |
| STOMA2006229 | 0 | 0 | 0 | 0 | 0 | 0 | 40.108 |
| SYNOV1000045 | 0 | 0 | 0 | 0 | 0 | 0 | 46.864 |
| SYNOV1000124 | 0 | 0 | 0 | 0 | 7.386 | 0 | 0 |
| SYNOV2001033 | 0 | 0 | 0 | 0 | 3.981 | 0 | 0 |
| SYNOV2009172 | 0 | 0 | 0 | 0 | 0 | 3.293 | 3.472 |
| T1ESE2000609 | 0 | 0 | 0 | 0 | 0 | 0 | 15.163 |
| TESOP2002451 | 0 | 51.382 | 0 | 0 | 0 | 0 | 0 |
| TESTI1000023 | 0 | 0 | 0 | 0 | 0 | 5.307 | 0 |
| TESTI2001099 | 0 | 0 | 0 | 0 | 3.253 | 3.101 | 0 |
| TESTI2001153 | 0 | 0 | 0 | 0 | 0 | 2.520 | 42.503 |
| TESTI2001269 | 0 | 0 | 0 | 0 | 0 | 3.454 | 0 |
| TESTI2001829 | 0 | 0 | 0 | 0 | 0 | 5.373 | 0 |
| TESTI2003325 | 0 | 0 | 0 | 0 | 0 | 0 | 28.233 |
| TESTI2003413 | 0 | 0 | 0 | 0 | 0 | 16.475 | 0 |
| TESTI2003533 | 0 | 18.755 | 0 | 0 | 0 | 0 | 0 |
| TESTI2003579 | 0 | 0 | 0 | 0 | 0 | 0 | 77.283 |
| TESTI2005759 | 0 | 20.230 | 0 | 0 | 0 | 0 | 0 |
| TESTI2005908 | 32.790 | 0 | 0 | 0 | 0 | 0 | 0 |
| TESTI2006051 | 0 | 0 | 0 | 9.107 | 0 | 0 | 0 |
| TESTI2006588 | 0 | 0 | 0 | 0 | 0 | 3.426 | 3.612 |
| TESTI2006748 | 0 | 0 | 0 | 0 | 0 | 21.767 | 0 |
| TESTI2007211 | 0 | 0 | 0 | 0 | 0 | 0 | 22.035 |
| TESTI2007464 | 0 | 0 | 0 | 0 | 0 | 25.379 | 26.758 |
| TESTI2008240 | 5.207 | 0 | 0 | 0 | 0 | 6.329 | 0 |
| TESTI2010513 | 0 | 0 | 0 | 0 | 0 | 2.486 | 0 |
| TESTI2011286 | 0 | 0 | 0 | 0 | 0 | 82.877 | 0 |
| TESTI2012511 | 0 | 92.868 | 0 | 0 | 0 | 0 | 0 |
| TESTI2012778 | 0 | 3.997 | 0 | 0 | 1.227 | 0 | 0 |
| TESTI2015213 | 0 | 0 | 0 | 0 | 0 | 3.247 | 0 |
| TESTI2017954 | 0 | 0 | 0 | 0 | 0 | 4.192 | 0 |
| TESTI2018997 | 0 | 0 | 0 | 0 | 0 | 13.178 | 0 |
| TESTI2021297 | 0 | 0 | 0 | 0 | 0 | 43.439 | 0 |
| TESTI2021425 | 0 | 0 | 0 | 37.027 | 0 | 0 | 0 |
| TESTI2024844 | 0 | 0 | 0 | 0 | 0 | 7.249 | 7.643 |
| TESTI2025422 | 0 | 0 | 0 | 0 | 0 | 0 | 16.559 |
| TESTI2028296 | 0 | 0 | 0 | 0 | 0 | 0 | 16.451 |
| TESTI2034940 | 0 | 5.635 | 0 | 5.603 | 0 | 0 | 5.214 |
| TESTI4000014 | 0 | 0 | 0 | 0 | 1.186 | 0 | 0 |
| TESTI4002799 | 0 | 9.553 | 0 | 0 | 0 | 0 | 0 |
| TESTI4010851 | 0 | 0 | 0 | 0 | 3.630 | 0 | 0 |
| TESTI4013441 | 0 | 3.393 | 0 | 0 | 2.083 | 2.977 | 1.046 |
| THYMU1000002 | 0 | 12.804 | 0 | 0 | 0 | 0 | 0 |
| THYMU1000109 | 0 | 0 | 0 | 16.927 | 0 | 0 | 0 |
| THYMU1000329 | 0 | 0 | 0 | 0 | 0 | 33.723 | 0 |
| THYMU2000382 | 0 | 0 | 0 | 13.036 | 0 | 0 | 0 |
| THYMU2000436 | 0 | 19.931 | 0 | 0 | 0 | 0 | 0 |
| THYMU2002756 | 0 | 0 | 0 | 0 | 48.606 | 0 | 0 |
| THYMU2003046 | 0 | 0 | 0 | 0 | 0 | 12.164 | 0 |
| THYMU2003133 | 0 | 0 | 0 | 0 | 23.036 | 0 | 0 |
| THYMU2004284 | 0 | 0 | 0 | 0 | 0 | 0 | 89.631 |
| THYMU2005003 | 0 | 0 | 0 | 0 | 0 | 88.356 | 0 |
| THYMU2005482 | 0 | 0 | 0 | 0 | 81.696 | 0 | 0 |
| THYMU2005980 | 0 | 0 | 0 | 0 | 0 | 0 | 54.098 |
| THYMU2008321 | 0 | 0 | 0 | 0 | 0 | 0 | 19.286 |
| THYMU2012625 | 0 | 22.471 | 0 | 0 | 0 | 0 | 0 |
| THYMU2027125 | 0 | 0 | 0 | 0 | 0 | 0 | 3.582 |
| THYMU2032155 | 0 | 0 | 0 | 0 | 0 | 45.186 | 0 |
| THYMU3033626 | 0 | 0 | 0 | 0 | 37.318 | 0 | 0 |
| THYMU3044188 | 0 | 0 | 0 | 96.268 | 0 | 0 | 0 |
| TKIDN2004185 | 0 | 0 | 0 | 85.316 | 0 | 0 | 0 |
| TKIDN2004748 | 0 | 0 | 0 | 0 | 64.204 | 0 | 0 |
| TKIDN2005487 | 0 | 0 | 0 | 0 | 4.770 | 0 | 0 |
| TKIDN2015788 | 54.776 | 0 | 0 | 0 | 0 | 0 | 0 |
| TRACH2000047 | 0 | 0 | 0 | 0 | 0 | 0 | 35.730 |
| TRACH2000079 | 0 | 0 | 0 | 0 | 3.597 | 0 | 0 |
| TRACH2000248 | 0 | 0 | 0 | 0 | 0 | 0 | 4.654 |
| TRACH2000321 | 0 | 0 | 0 | 0 | 3.262 | 0 | 0 |
| TRACH2000540 | 0 | 0 | 0 | 30.640 | 0 | 0 | 0 |
| TRACH2001021 | 0 | 0 | 0 | 0 | 0 | 0 | 2.793 |
| TRACH2001289 | 0 | 7.352 | 0 | 0 | 2.257 | 0 | 0 |
| TRACH2001607 | 0 | 0 | 0 | 0 | 0 | 0 | 6.700 |
| TRACH2005710 | 0 | 0 | 0 | 0 | 0 | 84.960 | 0 |
| TRACH2005811 | 0 | 0 | 0 | 0 | 0 | 6.673 | 0 |
| TRACH2007399 | 0 | 0 | 0 | 0 | 3.127 | 2.981 | 0 |
| TRACH2011113 | 0 | 0 | 0 | 0 | 0 | 29.874 | 0 |
| TRACH2014124 | 0 | 0 | 0 | 0 | 0 | 0 | 6.886 |
| TRACH2015824 | 0 | 0 | 0 | 49.981 | 0 | 0 | 0 |
| TRACH3002192 | 12.987 | 0 | 0 | 0 | 0 | 3.946 | 0 |
| TRACH3005294 | 77.338 | 0 | 0 | 0 | 0 | 0 | 0 |
| TRACH3007479 | 0 | 0 | 0 | 0 | 0 | 0 | 7.175 |
| UMVEN1000186 | 0 | 0 | 0 | 0 | 1.754 | 1.672 | 0 |
| UMVEN2000133 | 0 | 0 | 0 | 0 | 0 | 0 | 3.910 |
| UTERU1000182 | 0 | 0 | 0 | 0 | 0 | 0 | 5.412 |
| UTERU2000095 | 0 | 0 | 0 | 0 | 0 | 0 | 2.761 |
| UTERU2000263 | 0 | 0 | 0 | 0 | 0 | 3.242 | 0 |
| UTERU2000424 | 0 | 0 | 0 | 0 | 0 | 0 | 5.666 |
| UTERU2000485 | 0 | 0 | 0 | 0 | 84.772 | 0 | 0 |
| UTERU2001607 | 0 | 0 | 0 | 0 | 9.794 | 0 | 0 |
| UTERU2002294 | 0 | 0 | 0 | 0 | 0 | 11.486 | 0 |
| UTERU2004037 | 0 | 0 | 0 | 0 | 0 | 13.874 | 0 |
| UTERU2005292 | 0 | 0 | 0 | 66.969 | 0 | 0 | 0 |
| UTERU2006400 | 0 | 0 | 0 | 0 | 84.772 | 0 | 0 |
| UTERU2007924 | 0 | 57.855 | 0 | 0 | 0 | 0 | 0 |
| UTERU2008085 | 0 | 0 | 0 | 0 | 1.199 | 0 | 8.435 |
| UTERU2008938 | 0 | 0 | 0 | 0 | 0 | 0 | 16.418 |
| UTERU2009335 | 0 | 0 | 0 | 0 | 0 | 0 | 20.234 |
| UTERU2010417 | 0 | 0 | 0 | 0 | 0 | 1.995 | 0 |
| UTERU2012230 | 0 | 0 | 0 | 0 | 0 | 0 | 11.740 |
| UTERU2014898 | 0 | 32.915 | 0 | 0 | 0 | 0 | 0 |
| UTERU2015653 | 0 | 12.620 | 0 | 0 | 0 | 0 | 0 |
| UTERU2023045 | 0 | 0 | 0 | 0 | 0 | 57.268 | 0 |
| UTERU2023175 | 0 | 0 | 0 | 32.020 | 0 | 0 | 0 |

Genes whose expression levels were negligibly low in each of the above-described libraries are shown with their clone names below.

3NB691000018, 3NB691000085, 3NB691000113, 3NB691000116, 3NB691000191, 3NB692000012, 3NB692000029, 3NB692000102, 3NB692000154, 3NB692000276, 3NB692000305, 3NB692000330, 3NB692000374, 3NB692000429, 3NB692000484, 3NB692000529, 3NB692000545, 3NB692000973, 3NB692001034, 3NB692001040, 3NB692001123, 3NB692001267, 3NB692001288, 3NB692001339, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001526, 3NB692001538, 3NB692001548, 3NB692001557, 3NB692001637, 3NB692001719, 3NB692001853, 3NB692001861, 3NB692002051, 3NB692002365, 3NB692002685, 3NB692002806, 3NB692003538, 3NB692004045,

3NB692004724, 3NB692005439, 3NB692006952, 3NB692008178, 3NB692008729, ACTVT2000380, ADIPS1000064, ADIPS2000069, ADIPS2000088, ADIPS2000245, ADIPS2000425, ADRGL1000002, ADRGL1000018, ADRGL1000033, ADRGL1000038, ADRGL1000065, ADRGL1000067, ADRGL1000111, ADRGL1000144, ADRGL1000147, ADRGL1000160, ADRGL1000165, ADRGL1000182, ADRGL2000042, ADRGL2000056, ADRGL2000064, ADRGL2000074, ADRGL2000085, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830,

ADRGL2001836, ADRGL2001854, ADRGL2002013, ADRGL2002029, ADRGL2002191, ADRGL2002260, ADRGL2002392, ADRGL2002477, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283,

ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146, ADRGL2009273, ADRGL2009324, ADRGL2009489, ADRGL2009533, ADRGL2009691, ADRGL2009755, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO2000011, ASTRO2000014, ASTRO2000095, ASTRO2000141, ASTRO2000191, ASTRO2000278, ASTRO2000372, ASTRO2000381, ASTRO2000403, ASTRO2000417, ASTRO2000480, ASTRO2000482, ASTRO2000533, ASTRO2000653, ASTRO2000725, ASTRO2000801, ASTRO2000914,

ASTRO2001001, ASTRO2001008, ASTRO2001029, ASTRO2001088, ASTRO2001107, ASTRO2001122, ASTRO2001227, ASTRO2001249, ASTRO2001480, ASTRO2002024, ASTRO2002035, ASTRO2002064, ASTRO2002202, ASTRO2002459, ASTRO2002571, ASTRO2002632, ASTRO2002659, ASTRO2002693, ASTRO2002720, ASTRO2002733, ASTRO2002743, ASTRO2002842, ASTRO2003212, ASTRO2003316, ASTRO2003461, ASTRO2003574, ASTRO2003581, ASTRO2003740, ASTRO2003840, ASTRO2003925, ASTRO2003960, ASTRO2004032, ASTRO2004051, ASTRO2004177, ASTRO2004217, ASTRO2004297, ASTRO2004584, ASTRO2004628, ASTRO2004751, ASTRO2004877, ASTRO2004974, ASTRO2005008, ASTRO2005081, ASTRO2005096, ASTRO2005242, ASTRO2005273, ASTRO2005343, ASTRO2005360, ASTRO2005413, ASTRO2005423,

ASTRO2005553, ASTRO2005557, ASTRO2005575, ASTRO2005593, ASTRO2005687, ASTRO2005726, ASTRO2005863, ASTRO2005896, ASTRO2005902, ASTRO2006356, ASTRO2006475, ASTRO2006732, ASTRO2006747, ASTRO2006920, ASTRO2006952, ASTRO2007047, ASTRO2007221, ASTRO2007377, ASTRO2007515, ASTRO2007578, ASTRO2007643, ASTRO2007666, ASTRO2007948, ASTRO2007956, ASTRO2007962, ASTRO2008040, ASTRO2008216, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010819, ASTRO2010962, ASTRO2010996, ASTRO2011149,

ASTRO2011422, ASTRO2011426, ASTRO2011437, ASTRO2011461, ASTRO2011834, ASTRO2011893, ASTRO2012552, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014923, ASTRO2014961, ASTRO2015162, ASTRO2015185, ASTRO2015529, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018169, ASTRO2018373, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000123, BGGI11000193, BGGI11000285, BGGI12000022, BGGI12000067,

BGGI12000161, BGGI12000533, BGGI12000544, BGGI12000616, BGGI12000684, BGGI12001002, BGGI12001075, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722,

BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006830, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000137, BNGH41000153, BNGH41000169, BNGH41000190, BNGH41000198, BNGH41000216, BNGH41000235, BNGH42000130, BNGH42000132, BNGH42000405, BNGH42000532,

BNGH42000675, BNGH42000739, BNGH42000791, BNGH42000815, BNGH42000994, BNGH42001406, BNGH42001724, BNGH42002168, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42006135, BNGH42006226, BNGH42006234, BNGH42007037, BNGH42007366, BNGH42007594, BNGH42007788, BNGH42007798, BNGH42008199, BNGH42008510, BNGH42008603, BNGH42008649, BNGH42008743, BNGH42008832, BNGH42008850, BNGH42008943, BNGH42009021, BNGH42009025, BRACE1000020, BRACE1000047, BRACE1000051, BRACE1000065, BRACE1000073, BRACE1000084, BRACE1000087, BRACE1000092, BRACE1000093, BRACE1000101,

BRACE1000114, BRACE1000124, BRACE1000132, BRACE1000159, BRACE1000166, BRACE1000169, BRACE1000187, BRACE1000239, BRACE1000258, BRACE1000451, BRACE1000475, BRACE1000572, BRACE2000061, BRACE2000077, BRACE2000100, BRACE2000109, BRACE2000141, BRACE2000183, BRACE2000229, BRACE2000232, BRACE2000245, BRACE2000256, BRACE2000266, BRACE2000267, BRACE2000280, BRACE2000300, BRACE2000331, BRACE2000347, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750,

BRACE2000753, BRACE2000815, BRACE2000864, BRACE2000885, BRACE2000893, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001117, BRACE2001143, BRACE2001151, BRACE2001154, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001508, BRACE2001534, BRACE2001543, BRACE2001564, BRACE2001588, BRACE2001607, BRACE2001626,

BRACE2001673, BRACE2001677, BRACE2001692, BRACE2001705, BRACE2001709, BRACE2001711, BRACE2001726, BRACE2001733, BRACE2001737, BRACE2001742, BRACE2001777, BRACE2001779, BRACE2001834, BRACE2001865, BRACE2001872, BRACE2001881, BRACE2001892, BRACE2001898, BRACE2001923, BRACE2001942, BRACE2001944, BRACE2001954, BRACE2001972, BRACE2001973, BRACE2002026, BRACE2002034, BRACE2002050, BRACE2002081, BRACE2002091, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002394, BRACE2002409, BRACE2002431, BRACE2002441, BRACE2002444, BRACE2002458, BRACE2002467, BRACE2002468, BRACE2002478,

BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002661, BRACE2002676, BRACE2002677, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002807, BRACE2002812, BRACE2002834, BRACE2002849, BRACE2002857, BRACE2002860, BRACE2002861, BRACE2002884, BRACE2002896, BRACE2002948, BRACE2003037, BRACE2003078, BRACE2003097, BRACE2003110, BRACE2003168, BRACE2003199, BRACE2003210, BRACE2003217, BRACE2003254, BRACE2003269, BRACE2003285, BRACE2003319, BRACE2003398,

BRACE2003420, BRACE2003428, BRACE2003431, BRACE2003512, BRACE2003516, BRACE2003527, BRACE2003539, BRACE2003540, BRACE2003594, BRACE2003598, BRACE2003609, BRACE2003614, BRACE2003628, BRACE2003639, BRACE2003700, BRACE2003713, BRACE2003766, BRACE2003785, BRACE2003788, BRACE2003800, BRACE2003802, BRACE2003845, BRACE2003846, BRACE2003847, BRACE2003848, BRACE2003873, BRACE2003885, BRACE2003887, BRACE2003892, BRACE2003904, BRACE2003905, BRACE2003928, BRACE2003944, BRACE2003954, BRACE2003982, BRACE2003987, BRACE2005034, BRACE2005047, BRACE2005063, BRACE2005083, BRACE2005090, BRACE2005099, BRACE2005118, BRACE2005124, BRACE2005138, BRACE2005151, BRACE2005160, BRACE2005169, BRACE2005189, BRACE2005193,

BRACE2005196, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005395, BRACE2005408, BRACE2005433, BRACE2005434, BRACE2005448, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005949, BRACE2005981,

BRACE2005991, BRACE2006055, BRACE2006072, BRACE2006075, BRACE2006084, BRACE2006089, BRACE2006102, BRACE2006105, BRACE2006122, BRACE2006137, BRACE2006143, BRACE2006162, BRACE2006167, BRACE2006174, BRACE2006240, BRACE2006245, BRACE2006249, BRACE2006258, BRACE2006264, BRACE2006274, BRACE2006281, BRACE2006298, BRACE2006319, BRACE2006344, BRACE2006354, BRACE2006363, BRACE2006378, BRACE2006380, BRACE2006393, BRACE2006397, BRACE2006413, BRACE2006417, BRACE2006453, BRACE2006459, BRACE2006488, BRACE2006514, BRACE2006534, BRACE2006540, BRACE2006547, BRACE2006564, BRACE2006587, BRACE2006598, BRACE2006636, BRACE2006685, BRACE2006703, BRACE2006743, BRACE2006753, BRACE2006783, BRACE2006821, BRACE2006833,

BRACE2006859, BRACE2006871, BRACE2006909, BRACE2006911, BRACE2006924, BRACE2006935, BRACE2006937, BRACE2006951, BRACE2006982, BRACE2006986, BRACE2007013, BRACE2007064, BRACE2007068, BRACE2007087, BRACE2007089, BRACE2007109, BRACE2007138, BRACE2007174, BRACE2007191, BRACE2007197, BRACE2007201, BRACE2007203, BRACE2007232, BRACE2007254, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007518, BRACE2007522, BRACE2007527, BRACE2007538, BRACE2007563, BRACE2007567, BRACE2007577, BRACE2007621, BRACE2007640, BRACE2007641, BRACE2007646, BRACE2007663, BRACE2007671,

BRACE2007682, BRACE2007685, BRACE2007708, BRACE2007727, BRACE2007749, BRACE2007760, BRACE2007761, BRACE2007764, BRACE2007767, BRACE2007768, BRACE2007769, BRACE2007784, BRACE2007786, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008097, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008358, BRACE2008361, BRACE2008380, BRACE2008385, BRACE2008399, BRACE2008401, BRACE2008402, BRACE2008410, BRACE2008420, BRACE2008443,

BRACE2008473, BRACE2008480, BRACE2008481, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008706, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008941, BRACE2008948, BRACE2008960, BRACE2008999, BRACE2009014, BRACE2009016, BRACE2009031, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275,

BRACE2009291, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009517, BRACE2009533, BRACE2009558, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983,

BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317, BRACE2012510, BRACE2012625, BRACE2012653, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013911, BRACE2014108, BRACE2014161, BRACE2014232, BRACE2014257, BRACE2014268, BRACE2014306, BRACE2014475, BRACE2014657, BRACE2014746, BRACE2014780,

BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017410, BRACE2017438, BRACE2017574, BRACE2017580, BRACE2017587, BRACE2017720, BRACE2017844, BRACE2017872, BRACE2017929, BRACE2017934, BRACE2017985, BRACE2017992,

BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021014, BRACE2021143, BRACE2021148, BRACE2021245, BRACE2021254, BRACE2021264, BRACE2021344,

BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022638, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333,

BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345,

BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299,

BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381,

BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595,

BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004150, BRACE3004205, BRACE3004358, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005938, BRACE3005989,

BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079,

BRACE3010086, BRACE3010221, BRACE3010283, BRACE3010315, BRACE3010397, BRACE3010416, BRACE3010428, BRACE3010435, BRACE3010438, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170,

BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531,

BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771,

BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078,

BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376,

BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025,

BRAMY2000052, BRAMY2000076, BRAMY2000086, BRAMY2000151, BRAMY2000181, BRAMY2000196, BRAMY2000231, BRAMY2000277, BRAMY2000388, BRAMY2000508, BRAMY2000562, BRAMY2000585, BRAMY2000653, BRAMY2000814, BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287,

BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004363, BRAMY2004387, BRAMY2004492, BRAMY2004521, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466,

BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208,

BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200,

BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016327, BRAMY2016386, BRAMY2016706, BRAMY2016771, BRAMY2016892, BRAMY2016938, BRAMY2016953, BRAMY2017014,

BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019554, BRAMY2019600, BRAMY2019643, BRAMY2019710, BRAMY2019963, BRAMY2019985, BRAMY2019989, BRAMY2020050, BRAMY2020058, BRAMY2020106, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142,

BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2023939, BRAMY2024004, BRAMY2024073, BRAMY2024312,

BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451,

BRAMY2027461, BRAMY2027467, BRAMY2027587, BRAMY2027714, BRAMY2027717, BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028783, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032087, BRAMY2032231, BRAMY2032242, BRAMY2032290, BRAMY2032311, BRAMY2032317, BRAMY2032344, BRAMY2032589, BRAMY2032678,

BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100,

BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317,

BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3002991, BRAMY3003026, BRAMY3003109, BRAMY3003205,

BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010492, BRAMY3010603, BRAMY3010654, BRAMY4000089, BRAMY4000095,

BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000007, BRAWH1000045, BRAWH1000083, BRAWH1000093, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417,

BRAWH2000443, BRAWH2000460, BRAWH2000471, BRAWH2000476, BRAWH2000482, BRAWH2000488, BRAWH2000494, BRAWH2000500, BRAWH2000503, BRAWH2000522, BRAWH2000554, BRAWH2000595, BRAWH2000633, BRAWH2000643, BRAWH2000651, BRAWH2000682, BRAWH2000686, BRAWH2000697, BRAWH2000752, BRAWH2000859, BRAWH2000866, BRAWH2000892, BRAWH2000901, BRAWH2000926, BRAWH2000944, BRAWH2000984, BRAWH2001037, BRAWH2001052, BRAWH2001103, BRAWH2001129, BRAWH2001137, BRAWH2001141, BRAWH2001159, BRAWH2001166, BRAWH2001171, BRAWH2001186, BRAWH2001203, BRAWH2001238, BRAWH2001239, BRAWH2001255, BRAWH2001321, BRAWH2001355, BRAWH2001378, BRAWH2001395, BRAWH2001406, BRAWH2001412, BRAWH2001418, BRAWH2001424, BRAWH2001436, BRAWH2001438,

BRAWH2001439, BRAWH2001459, BRAWH2001461, BRAWH2001484, BRAWH2001492, BRAWH2001503, BRAWH2001519, BRAWH2001530, BRAWH2001535, BRAWH2001589, BRAWH2001662, BRAWH2001666, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002967, BRAWH2002976, BRAWH2003000, BRAWH2003025, BRAWH2003135, BRAWH2003240, BRAWH2003307, BRAWH2003355,

BRAWH2003366, BRAWH2003662, BRAWH2003689, BRAWH2003693, BRAWH2003818, BRAWH2003832, BRAWH2003948, BRAWH2003964, BRAWH2004044, BRAWH2004068, BRAWH2004078, BRAWH2004095, BRAWH2004136, BRAWH2004217, BRAWH2004580, BRAWH2004731, BRAWH2004779, BRAWH2004842, BRAWH2004884, BRAWH2004923, BRAWH2005008, BRAWH2005074, BRAWH2005225, BRAWH2005315, BRAWH2005517, BRAWH2005524, BRAWH2005533, BRAWH2005578, BRAWH2005661, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2006996, BRAWH2007069, BRAWH2007308, BRAWH2007406,

BRAWH2007550, BRAWH2007570, BRAWH2007591, BRAWH2007605, BRAWH2007658, BRAWH2007664, BRAWH2007800, BRAWH2007808, BRAWH2007825, BRAWH2007862, BRAWH2007890, BRAWH2008058, BRAWH2008128, BRAWH2008219, BRAWH2008255, BRAWH2008292, BRAWH2008297, BRAWH2008366, BRAWH2008380, BRAWH2008540, BRAWH2008577, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010536,

BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014188,

BRAWH2014234, BRAWH2014379, BRAWH2014414, BRAWH2014473, BRAWH2014511, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534,

BRAWH2017542, BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018729, BRAWH2018745, BRAWH2018875, BRAWH2019053, BRAWH2019055, BRAWH2019198, BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002574, BRAWH3002600, BRAWH3002819, BRAWH3003019, BRAWH3003136, BRAWH3003244,

BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461,

BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884,

BRAWH3020928, BRAWH3021012, BRAWH3021545, BRAWH3021574, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806,

BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN2000148, BRCAN2000149,

BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001223, BRCAN2001866, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401,

BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2009432, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886,

BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368, BRCAN2025712, BRCAN2025982, BRCAN2026117, BRCAN2026340,

BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028040, BRCAN2028319, BRCAN2028338, BRCAN2028355, BRCAN2028378, BRCAN2028460, BRCAN2028545, BRCAN2028634, BRCAN2028702, BRCOC1000040, BRCOC1000087, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003213, BRCOC2003513, BRCOC2003732,

BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010123, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014013,

BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018877, BRCOC2019255, BRCOC2019549, BRCOC2019841, BRCOC2019896, BRCOC2019903, BRCOC2019910, BRCOC2019934, BRCOC2020122, BRCOC2020142, BRHIP1000058, BRHIP1000072, BRHIP1000104, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000553, BRHIP2000621, BRHIP2000691, BRHIP2000819, BRHIP2000826, BRHIP2000920, BRHIP2001074, BRHIP2001099,

BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007741, BRHIP2007928, BRHIP2007969, BRHIP2008269,

BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958,

BRHIP2013972, BRHIP2014063, BRHIP2014228, BRHIP2014285, BRHIP2014291, BRHIP2014373, BRHIP2014386, BRHIP2014387, BRHIP2014954, BRHIP2015153, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019819, BRHIP2019884,

BRHIP2019972, BRHIP2020182, BRHIP2020509, BRHIP2020573, BRHIP2020622, BRHIP2020695, BRHIP2020743, BRHIP2020799, BRHIP2020842, BRHIP2020859, BRHIP2020930, BRHIP2021102, BRHIP2021289, BRHIP2021495, BRHIP2021615, BRHIP2021744, BRHIP2021762, BRHIP2021858, BRHIP2021870, BRHIP2021929, BRHIP2022006, BRHIP2022054, BRHIP2022221, BRHIP2022228, BRHIP2022298, BRHIP2022467, BRHIP2022708, BRHIP2022986, BRHIP2023071, BRHIP2023229, BRHIP2023272, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024398, BRHIP2024549, BRHIP2024742, BRHIP2024789, BRHIP2024911,

BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000017, BRHIP3000060, BRHIP3000070, BRHIP3000081, BRHIP3000111, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076,

BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683,

BRHIP3006717, BRHIP3006786, BRHIP3006950, BRHIP3007172, BRHIP3007183, BRHIP3007195, BRHIP3007223, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745,

BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3018784, BRHIP3019643, BRHIP3019824, BRHIP3019880, BRHIP3019956, BRHIP3020046, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844,

BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000498, BRSSN2000518, BRSSN2000561, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2001869, BRSSN2002857, BRSSN2002859, BRSSN2003012, BRSSN2003086, BRSSN2003093, BRSSN2003820, BRSSN2003841,

BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009518, BRSSN2009627, BRSSN2009630, BRSSN2009702, BRSSN2010019, BRSSN2010110, BRSSN2010587, BRSSN2010596, BRSSN2010830, BRSSN2011262, BRSSN2011653, BRSSN2011738, BRSSN2011843, BRSSN2012081, BRSSN2012103, BRSSN2012157, BRSSN2012198,

BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014610, BRSSN2014636, BRSSN2014685, BRSSN2015199, BRSSN2015238, BRSSN2015369, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000083, BRSTN1000097, BRSTN2000058, BRSTN2000070,

BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284,

BRSTN2012320, BRSTN2012380, BRSTN2012451, BRSTN2013171, BRSTN2013354, BRSTN2013502, BRSTN2013931, BRSTN2014490, BRSTN2014633, BRSTN2014751, BRSTN2014770, BRSTN2015015, BRSTN2015889, BRSTN2015978, BRSTN2016066, BRSTN2016335, BRSTN2016892, BRSTN2016918, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133,

BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005864, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665,

BRTHA2007998, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562, BRTHA2012980, BRTHA2013054, BRTHA2013262, BRTHA2013275, BRTHA2013426, BRTHA2013460, BRTHA2013515, BRTHA2013540,

BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016577, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014,

BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448,

BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003490, BRTHA3003704, BRTHA3003736, BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113,

BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011194, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908,

BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3021971, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152,

CD34C2000175, CD34C2000443, CD34C3000125, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, CHONS2002829, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337,

COLON2009499, CORDB1000140, CORDB2000061, CORDB2000127, CORDB2000541, CTONG1000009, CTONG1000010, CTONG1000020, CTONG1000022, CTONG1000040, CTONG1000052, CTONG1000056, CTONG1000062, CTONG1000086, CTONG1000088, CTONG1000093, CTONG1000094, CTONG1000097, CTONG1000113, CTONG1000137, CTONG1000165, CTONG1000180, CTONG1000181, CTONG1000218, CTONG1000220, CTONG1000241, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042, CTONG2000063, CTONG2000201, CTONG2000218, CTONG2000279, CTONG2000303, CTONG2000330, CTONG2000395, CTONG2000411, CTONG2000465, CTONG2000469, CTONG2000480,

CTONG2000508, CTONG2000561, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001366, CTONG2001413, CTONG2001428, CTONG2001513, CTONG2001524, CTONG2001533, CTONG2001598, CTONG2001681, CTONG2001724, CTONG2001749, CTONG2001800, CTONG2001820, CTONG2001858, CTONG2001877, CTONG2001911, CTONG2001932, CTONG2001955, CTONG2002066, CTONG2002095, CTONG2002143, CTONG2002270, CTONG2002395, CTONG2002417, CTONG2002418, CTONG2002453, CTONG2002558, CTONG2002590, CTONG2002709, CTONG2002721, CTONG2002744, CTONG2002766, CTONG2002803, CTONG2002816, CTONG2002820, CTONG2002832, CTONG2002841, CTONG2002903,

CTONG2002965, CTONG2002999, CTONG2003028, CTONG2003094, CTONG2003100, CTONG2003115, CTONG2003189, CTONG2003245, CTONG2003293, CTONG2003348, CTONG2003350, CTONG2003361, CTONG2003372, CTONG2003375, CTONG2003427, CTONG2003517, CTONG2003524, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004423, CTONG2004454, CTONG2004487, CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005278, CTONG2005290, CTONG2005374, CTONG2005399,

CTONG2005468, CTONG2005553, CTONG2005567, CTONG2005585, CTONG2005615, CTONG2005635, CTONG2005775, CTONG2005795, CTONG2005890, CTONG2005904, CTONG2005913, CTONG2006129, CTONG2006223, CTONG2006235, CTONG2006273, CTONG2006310, CTONG2006377, CTONG2006393, CTONG2006412, CTONG2006449, CTONG2006515, CTONG2006524, CTONG2006568, CTONG2006611, CTONG2006620, CTONG2006666, CTONG2006691, CTONG2006709, CTONG2006798, CTONG2006836, CTONG2006942, CTONG2006964, CTONG2007007, CTONG2007009, CTONG2007072, CTONG2007078, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007500,

CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008269, CTONG2008321, CTONG2008343, CTONG2008398, CTONG2008402, CTONG2008405, CTONG2008466, CTONG2008518, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604,

CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010348, CTONG2010408, CTONG2010508, CTONG2010566, CTONG2010623, CTONG2010649, CTONG2010652, CTONG2010803, CTONG2011188, CTONG2011429, CTONG2011474, CTONG2011676, CTONG2011716, CTONG2011765, CTONG2011779, CTONG2011801, CTONG2011815, CTONG2011822, CTONG2011825, CTONG2011920, CTONG2011985, CTONG2012001, CTONG2012029, CTONG2012050, CTONG2012077, CTONG2012101, CTONG2012123, CTONG2012158,

CTONG2012230, CTONG2012401, CTONG2012422, CTONG2012425, CTONG2012447, CTONG2012453, CTONG2012473, CTONG2012476, CTONG2012533, CTONG2012554, CTONG2012607, CTONG2012661, CTONG2012724, CTONG2012738, CTONG2012758, CTONG2012843, CTONG2012847, CTONG2012879, CTONG2012901, CTONG2012996, CTONG2013128, CTONG2013149, CTONG2013156, CTONG2013178, CTONG2013222, CTONG2013284, CTONG2013339, CTONG2013348, CTONG2013352, CTONG2013381, CTONG2013393, CTONG2013511, CTONG2013630, CTONG2013803, CTONG2013934, CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014369, CTONG2014389, CTONG2014491, CTONG2014630,

CTONG2014635, CTONG2014646, CTONG2014705, CTONG2014898, CTONG2014946, CTONG2014954, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016056, CTONG2016185, CTONG2016217, CTONG2016355, CTONG2016408, CTONG2016499, CTONG2016505, CTONG2016559, CTONG2016658, CTONG2016775, CTONG2016797, CTONG2016846, CTONG2016869, CTONG2016904, CTONG2016942, CTONG2016953, CTONG2017021, CTONG2017094, CTONG2017292, CTONG2017409, CTONG2017429, CTONG2017444, CTONG2017458, CTONG2017604,

CTONG2017650, CTONG2017798, CTONG2017804, CTONG2017814, CTONG2017910, CTONG2017939, CTONG2017989, CTONG2017998, CTONG2018062, CTONG2018069, CTONG2018147, CTONG2018211, CTONG2018217, CTONG2018279, CTONG2018334, CTONG2018343, CTONG2018379, CTONG2018383, CTONG2018413, CTONG2018483, CTONG2018629, CTONG2018632, CTONG2018637, CTONG2018652, CTONG2018653, CTONG2018655, CTONG2018735, CTONG2018739, CTONG2018808, CTONG2018843, CTONG2018898, CTONG2018976, CTONG2019029, CTONG2019063, CTONG2019144, CTONG2019232, CTONG2019248, CTONG2019400, CTONG2019590, CTONG2019652, CTONG2019704, CTONG2019788, CTONG2019822, CTONG2019833, CTONG2020031, CTONG2020108, CTONG2020127, CTONG2020171, CTONG2020374, CTONG2020378,

CTONG2020411, CTONG2020445, CTONG2020522, CTONG2020560, CTONG2020582, CTONG2020638, CTONG2020806, CTONG2020831, CTONG2020974, CTONG2021024, CTONG2021132, CTONG2021168, CTONG2021289, CTONG2022153, CTONG2022601, CTONG2022835, CTONG2023021, CTONG2023449, CTONG2023512, CTONG2024031, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028309, CTONG2028486, CTONG2028518, CTONG2028687, CTONG2028758, CTONG3000686, CTONG3000707, CTONG3000896,

CTONG3001123, CTONG3001420, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002674, CTONG3002728, CTONG3002835, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659,

CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008894, CTONG3008951, CTONG3008959, CTONG3009227, CTONG3009239, CTONG3009287, CTONG3009328, CTONG3009385, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001670, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000195, D3OST3000258, D3OST3000291,

D3OST3000388, D3OST3000493, D6OST2000127, D6OST2000169, D6OST2000245, D6OST2000358, D6OST2000507, D9OST2000031, D9OST2000278, D9OST2000440, D9OST2000869, D9OST2001319, D9OST2001433, D9OST2002397, D9OST2002608, D9OST2002673, D9OST2003106, D9OST2003108, D9OST2003137, D9OST2003580, D9OST2003594, D9OST2003762, D9OST2003791, D9OST2003989, D9OST2004018, D9OST2004073, D9OST2004417, DFNES1000003, DFNES1000083, DFNES1000107, DFNES1000150, DFNES1000185, DFNES2000143, DFNES2000146, DFNES2000153, DFNES2000212, DFNES2000268, DFNES2000292, DFNES2000335, DFNES2000426, DFNES2000432, DFNES2000443, DFNES2000457, DFNES2000471, DFNES2000526, DFNES2000913, DFNES2001091, DFNES2001096, DFNES2001108, DFNES2001177,

DFNES2001404, DFNES2001564, DFNES2001565, DFNES2001647, DFNES2001800, DFNES2001829, DFNES2002220, DFNES2002550, DFNES2002588, DFNES2002817, DFNES2002966, DFNES2003081, DFNES2003192, DFNES2003255, DFNES2003742, DFNES2004346, DFNES2004354, DFNES2004371, DFNES2004383, DFNES2004608, DFNES2004684, DFNES2005266, DFNES2005527, DFNES2005690, DFNES2005779, DFNES2005875, DFNES2006346, DFNES2006944, DFNES2007113, DFNES2007299, DFNES2007634, DFNES2007641, DFNES2007757, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000023, FCBBF1000024,

FCBBF1000025, FCBBF1000038, FCBBF1000053, FCBBF1000061, FCBBF1000063, FCBBF1000069, FCBBF1000077, FCBBF1000115, FCBBF1000117, FCBBF1000118, FCBBF1000121, FCBBF1000125, FCBBF1000131, FCBBF1000155, FCBBF1000157, FCBBF1000171, FCBBF1000174, FCBBF1000182, FCBBF1000197, FCBBF1000198, FCBBF1000206, FCBBF1000220, FCBBF1000232, FCBBF1000233, FCBBF1000243, FCBBF1000270, FCBBF1000272, FCBBF1000294, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000403, FCBBF1000412, FCBBF1000425, FCBBF1000437, FCBBF1000449, FCBBF1000466, FCBBF1000476, FCBBF1000506, FCBBF1000509, FCBBF1000546, FCBBF1000550, FCBBF1000556,

FCBBF1000563, FCBBF1000574, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000748, FCBBF1000760, FCBBF2000038, FCBBF2000087, FCBBF2000094, FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566, FCBBF2000576, FCBBF2000591, FCBBF2000677, FCBBF2000678, FCBBF2000685, FCBBF2000733, FCBBF2000782, FCBBF2000808, FCBBF2000815, FCBBF2000825, FCBBF2000885, FCBBF2000940, FCBBF2000951, FCBBF2001001, FCBBF2001088, FCBBF2001105, FCBBF2001116,

FCBBF2001183, FCBBF2001188, FCBBF2001211, FCBBF2001238, FCBBF2001243, FCBBF2001299, FCBBF2001309, FCBBF2001400, FCBBF2001427, FCBBF2001480, FCBBF2001529, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002349, FCBBF2002370, FCBBF2002541, FCBBF2002626, FCBBF2002801, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373,

FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005733, FCBBF2005909, FCBBF2005966, FCBBF2006131, FCBBF2006380, FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000110, FCBBF3000115, FCBBF3000120, FCBBF3000168, FCBBF3000175, FCBBF3000227, FCBBF3000228, FCBBF3000229, FCBBF3000233, FCBBF3000269, FCBBF3000361, FCBBF3000367, FCBBF3000388, FCBBF3000434, FCBBF3000462, FCBBF3000473, FCBBF3000501, FCBBF3000518, FCBBF3000536, FCBBF3000550, FCBBF3000847,

FCBBF3001018, FCBBF3001020, FCBBF3001081, FCBBF3001235, FCBBF3001281, FCBBF3001302, FCBBF3001377, FCBBF3001594, FCBBF3001657, FCBBF3001818, FCBBF3001855, FCBBF3001890, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004261, FCBBF3004323, FCBBF3004390, FCBBF3004409, FCBBF3004473, FCBBF3004502, FCBBF3004715, FCBBF3004842, FCBBF3004847, FCBBF3004955, FCBBF3005160,

FCBBF3005218, FCBBF3005444, FCBBF3005497, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008153, FCBBF3008159, FCBBF3008251, FCBBF3008311, FCBBF3008362, FCBBF3008382, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479,

FCBBF3009526, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800,

FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016987, FCBBF3017024, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018796, FCBBF3018826, FCBBF3018949, FCBBF3019085, FCBBF3019320, FCBBF3019437, FCBBF3019564,

FCBBF3019569, FCBBF3019570, FCBBF3019663, FCBBF3019716, FCBBF3019784, FCBBF3019839, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3023061, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285,

FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028244, FCBBF3028472, FCBBF3028528, FCBBF3028582, FCBBF3028593, FCBBF3028671, FCBBF3028794, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000500, FCBBF4000529, FCBBF4000548, FCBBF5000041,

FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000022, FEBRA1000030, FEBRA1000057, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000105, FEBRA2000126, FEBRA2000210, FEBRA2000220, FEBRA2000228, FEBRA2000253, FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000452,

FEBRA2000454, FEBRA2000462, FEBRA2000468, FEBRA2000491, FEBRA2000504, FEBRA2000510, FEBRA2000532, FEBRA2000536, FEBRA2000538, FEBRA2000575, FEBRA2000581, FEBRA2000656, FEBRA2000659, FEBRA2000680, FEBRA2000690, FEBRA2000727, FEBRA2000734, FEBRA2000757, FEBRA2000762, FEBRA2000771, FEBRA2000772, FEBRA2000782, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000881, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175,

FEBRA2001197, FEBRA2001217, FEBRA2001227, FEBRA2001245, FEBRA2001267, FEBRA2001334, FEBRA2001351, FEBRA2001383, FEBRA2001438, FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001752, FEBRA2001772, FEBRA2001790, FEBRA2001814, FEBRA2001828, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002429, FEBRA2002442, FEBRA2002508,

FEBRA2002525, FEBRA2002527, FEBRA2002552, FEBRA2002574, FEBRA2002601, FEBRA2002611, FEBRA2002628, FEBRA2002662, FEBRA2002682, FEBRA2002707, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003100, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003750, FEBRA2003822, FEBRA2003833,

FEBRA2003897, FEBRA2003907, FEBRA2003930, FEBRA2004023, FEBRA2004026, FEBRA2004029, FEBRA2004042, FEBRA2004053, FEBRA2004056, FEBRA2004091, FEBRA2004110, FEBRA2004191, FEBRA2004202, FEBRA2004219, FEBRA2004237, FEBRA2004268, FEBRA2004293, FEBRA2004325, FEBRA2004329, FEBRA2004331, FEBRA2004412, FEBRA2004443, FEBRA2004485, FEBRA2004490, FEBRA2004538, FEBRA2004592, FEBRA2004620, FEBRA2004628, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005416, FEBRA2005427, FEBRA2005458,

FEBRA2005476, FEBRA2005701, FEBRA2005726, FEBRA2005752, FEBRA2005778, FEBRA2005787, FEBRA2005788, FEBRA2005824, FEBRA2005995, FEBRA2005998, FEBRA2006055, FEBRA2006061, FEBRA2006092, FEBRA2006112, FEBRA2006150, FEBRA2006165, FEBRA2006255, FEBRA2006270, FEBRA2006315, FEBRA2006346, FEBRA2006354, FEBRA2006357, FEBRA2006368, FEBRA2006370, FEBRA2006372, FEBRA2006379, FEBRA2006396, FEBRA2006401, FEBRA2006409, FEBRA2006418, FEBRA2006427, FEBRA2006476, FEBRA2006485, FEBRA2006491, FEBRA2006519, FEBRA2006521, FEBRA2006553, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017,

FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007620, FEBRA2007708, FEBRA2007714, FEBRA2007793, FEBRA2007801, FEBRA2007818, FEBRA2007823, FEBRA2007900, FEBRA2007901, FEBRA2007931, FEBRA2007957, FEBRA2008009, FEBRA2008036, FEBRA2008081, FEBRA2008086, FEBRA2008134, FEBRA2008135, FEBRA2008159, FEBRA2008165, FEBRA2008172, FEBRA2008201, FEBRA2008210, FEBRA2008255, FEBRA2008265, FEBRA2008266, FEBRA2008282, FEBRA2008302, FEBRA2008311, FEBRA2008341, FEBRA2008360, FEBRA2008459, FEBRA2008468, FEBRA2008475, FEBRA2008481, FEBRA2008542,

FEBRA2008660, FEBRA2008662, FEBRA2008681, FEBRA2008692, FEBRA2008741, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009437, FEBRA2009478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063,

FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502,

FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2018476, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019711, FEBRA2019867, FEBRA2020406, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022148, FEBRA2022204, FEBRA2022255, FEBRA2022322, FEBRA2022601,

FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023377, FEBRA2023498, FEBRA2023550, FEBRA2023721, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025463, FEBRA2025477, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEBRA2028516, HELAC2000158,

HELAC2000301, HHDPC1000001, HHDPC1000065, HHDPC1000118, HHDPC2000055, HHDPC2000070, HHDPC2000102, HHDPC2000104, HHDPC2000149, HHDPC2000258, HHDPC2000315, HHDPC2000455, HHDPC2000456, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003472, HHDPC2004757, HHDPC2005185, HHDPC2005513, HHDPC2005742, HHDPC2005794, HHDPC2006460, HHDPC2006862, HHDPC2006927, HHDPC2007267, HHDPC2007302, HHDPC2007775, HHDPC2008123, HHDPC2008185, HHDPC2008279, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030,

HLUNG1000034, HLUNG1000037, HLUNG1000053, HLUNG1000064, HLUNG1000076, HLUNG1000084, HLUNG1000091, HLUNG1000099, HLUNG1000104, HLUNG1000105, HLUNG1000110, HLUNG1000151, HLUNG1000169, HLUNG2000004, HLUNG2000014, HLUNG2000027, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363, HLUNG2000412, HLUNG2000431, HLUNG2000480, HLUNG2000489, HLUNG2000549, HLUNG2000557, HLUNG2000570, HLUNG2000751, HLUNG2000761, HLUNG2000777, HLUNG2000846, HLUNG2000870, HLUNG2000884, HLUNG2000926, HLUNG2000950, HLUNG2000955, HLUNG2000970, HLUNG2001013,

HLUNG2001126, HLUNG2001144, HLUNG2001146, HLUNG2001214, HLUNG2001439, HLUNG2001459, HLUNG2001507, HLUNG2001518, HLUNG2001633, HLUNG2001677, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002562, HLUNG2002648, HLUNG2002659, HLUNG2002707, HLUNG2002811, HLUNG2002916, HLUNG2002942, HLUNG2002949, HLUNG2002958, HLUNG2002982, HLUNG2003003, HLUNG2003042, HLUNG2003049, HLUNG2003061, HLUNG2003162, HLUNG2003246, HLUNG2003306, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918,

HLUNG2003991, HLUNG2004014, HLUNG2004037, HLUNG2004067, HLUNG2004154, HLUNG2004159, HLUNG2004170, HLUNG2004217, HLUNG2004273, HLUNG2004388, HLUNG2004416, HLUNG2004521, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006812, HLUNG2006843, HLUNG2006852, HLUNG2006935, HLUNG2007041, HLUNG2007163, HLUNG2007219, HLUNG2007433, HLUNG2007931, HLUNG2008066,

HLUNG2008139, HLUNG2008153, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008637, HLUNG2008833, HLUNG2008875, HLUNG2008885, HLUNG2008958, HLUNG2008959, HLUNG2009067, HLUNG2009215, HLUNG2009225, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288,

HLUNG2014449, HLUNG2014821, HLUNG2015184, HLUNG2015418, HLUNG2015548, HLUNG2015578, HLUNG2015617, HLUNG2016022, HLUNG2016862, HLUNG2017262, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000137, HSYRA1000148, HSYRA1000152, HSYRA1000178, HSYRA2000044, HSYRA2000135, HSYRA2000190, HSYRA2000221, HSYRA2000237, HSYRA2000253, HSYRA2000371, HSYRA2000424, HSYRA2000507, HSYRA2000510, HSYRA2000605, HSYRA2000629, HSYRA2000640, HSYRA2000706, HSYRA2000760, HSYRA2000787, HSYRA2000828, HSYRA2000832, HSYRA2000925, HSYRA2000927, HSYRA2001003, HSYRA2001105, HSYRA2001142, HSYRA2001153, HSYRA2001225, HSYRA2001255,

HSYRA2001332, HSYRA2001353, HSYRA2001396, HSYRA2001420, HSYRA2001484, HSYRA2001552, HSYRA2001580, HSYRA2001595, HSYRA2001631, HSYRA2001638, HSYRA2001983, HSYRA2002103, HSYRA2002590, HSYRA2002741, HSYRA2003168, HSYRA2004550, HSYRA2004858, HSYRA2005456, HSYRA2005496, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000161, IMR321000165, IMR321000193, IMR321000207, IMR321000210, IMR321000219, IMR321000230, IMR321000242, IMR321000253, IMR321000266, IMR322000010, IMR322000107, IMR322000121, IMR322000127, IMR322000144, IMR322000152, IMR322000164, IMR322000243, IMR322000302, IMR322000316,

IMR322000379, IMR322000383, IMR322000418, IMR322000518, IMR322000604, IMR322000609, IMR322000656, IMR322000672, IMR322000775, IMR322000811, IMR322000819, IMR322000838, IMR322000859, IMR322000863, IMR322000917, IMR322000918, IMR322000919, IMR322000935, IMR322000953, IMR322000973, IMR322000984, IMR322001018, IMR322001049, IMR322001167, IMR322001185, IMR322001218, IMR322001245, IMR322001318, IMR322001322, IMR322001332, IMR322001380, IMR322001438, IMR322001452, IMR322001491, IMR322001541, IMR322001584, IMR322001665, IMR322001670, IMR322001724, IMR322001879, IMR322002035, IMR322002110, IMR322002470, IMR322002993, IMR322003675, IMR322004768, IMR322005293, IMR322005833, IMR322006012, IMR322006222,

IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, IMR322019070, JCMLC2000273, KIDNE1000012, KIDNE1000015, KIDNE1000028, KIDNE1000036, KIDNE1000064, KIDNE1000104, KIDNE1000121, KIDNE1000145, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000252, KIDNE2000266, KIDNE2000287, KIDNE2000317,

KIDNE2000328, KIDNE2000330, KIDNE2000335, KIDNE2000349, KIDNE2000375, KIDNE2000383, KIDNE2000394, KIDNE2000422, KIDNE2000493, KIDNE2000510, KIDNE2000513, KIDNE2000517, KIDNE2000519, KIDNE2000555, KIDNE2000574, KIDNE2000624, KIDNE2000678, KIDNE2000717, KIDNE2000721, KIDNE2000722, KIDNE2000777, KIDNE2000801, KIDNE2000833, KIDNE2000846, KIDNE2000867, KIDNE2000947, KIDNE2001117, KIDNE2001140, KIDNE2001162, KIDNE2001269, KIDNE2001361, KIDNE2001373, KIDNE2001636, KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002252, KIDNE2002262, KIDNE2002265,

KIDNE2002438, KIDNE2002483, KIDNE2002725, KIDNE2002768, KIDNE2002795, KIDNE2002798, KIDNE2002845, KIDNE2002882, KIDNE2002991, KIDNE2003185, KIDNE2003188, KIDNE2003233, KIDNE2003305, KIDNE2003316, KIDNE2003335, KIDNE2003357, KIDNE2003373, KIDNE2003377, KIDNE2003752, KIDNE2003764, KIDNE2003837, KIDNE2003941, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411, KIDNE2004475, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004864, KIDNE2004879,

KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006014, KIDNE2006030, KIDNE2006039, KIDNE2006053, KIDNE2006092, KIDNE2006145, KIDNE2006149, KIDNE2006210, KIDNE2006299, KIDNE2006334, KIDNE2006353, KIDNE2006376, KIDNE2006378, KIDNE2006465, KIDNE2006580, KIDNE2006652, KIDNE2006687, KIDNE2006733, KIDNE2006760, KIDNE2006775, KIDNE2006811, KIDNE2006820, KIDNE2006880, KIDNE2007005, KIDNE2007040, KIDNE2007077,

KIDNE2007186, KIDNE2007222, KIDNE2007328, KIDNE2007352, KIDNE2007356, KIDNE2007422, KIDNE2007569, KIDNE2007810, KIDNE2007811, KIDNE2007910, KIDNE2007944, KIDNE2007954, KIDNE2008022, KIDNE2008069, KIDNE2008072, KIDNE2008116, KIDNE2008117, KIDNE2008218, KIDNE2008315, KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009367, KIDNE2009426, KIDNE2009467, KIDNE2009490, KIDNE2009553, KIDNE2009605, KIDNE2009628,

KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012440, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263,

KIDNE2013346, KIDNE2013388, KIDNE2013413, KIDNE2013489, KIDNE2013639, KIDNE2013731, KIDNE2013734, KIDNE2013775, KIDNE2013801, KIDNE2013845, KIDNE2013866, KIDNE2014087, KIDNE2014112, KIDNE2014119, KIDNE2014170, KIDNE2014184, KIDNE2014268, KIDNE2014298, KIDNE2014320, KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327,

KIDNE2016371, KIDNE2016388, KIDNE2016464, KIDNE2016539, KIDNE2016570, KIDNE2016918, KIDNE2017007, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018352, KIDNE2018462, KIDNE2018493, KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000079, LIVER1000082, LIVER1000099, LIVER1000104, LIVER1000111, LIVER1000126,

LIVER1000132, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000033, LIVER2000037, LIVER2000158, LIVER2000216, LIVER2000237, LIVER2000247, LIVER2000360, LIVER2000416, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2001608, LIVER2001835, LIVER2002638, LIVER2002644, LIVER2002660, LIVER2002702, LIVER2002842, LIVER2003008,

LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006006, LIVER2006251, LIVER2006410, LIVER2006469, LIVER2006644, LIVER2006764, LIVER2007415, LIVER2007548, LIVER2007568, LIVER2007721, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017,

MAMGL1000083, MAMGL1000096, MAMGL1000100, MAMGL1000132, MAMGL1000151, MAMGL1000178, MAMGL1000182, MAMGL1000184, MESAN1000032, MESAN1000035, MESAN1000050, MESAN1000079, MESAN1000101, MESAN1000121, MESAN1000147, MESAN1000180, MESAN2000023, MESAN2000026, MESAN2000067, MESAN2000092, MESAN2000149, MESAN2000167, MESAN2000264, MESAN2000291, MESAN2000337, MESAN2000434, MESAN2000457, MESAN2000462, MESAN2000501, MESAN2000620, MESAN2000711, MESAN2000815, MESAN2000894, MESAN2001154, MESAN2001450, MESAN2001627, MESAN2001770, MESAN2001979, MESAN2001980, MESAN2002113, MESAN2002122, MESAN2002147, MESAN2002186, MESAN2002424, MESAN2002519, MESAN2002687, MESAN2002709, MESAN2002724, MESAN2002844, MESAN2002940,

MESAN2002978, MESAN2003035, MESAN2003037, MESAN2003039, MESAN2003058, MESAN2003101, MESAN2003190, MESAN2003322, MESAN2003444, MESAN2003490, MESAN2003529, MESAN2003646, MESAN2003662, MESAN2003709, MESAN2003851, MESAN2004138, MESAN2004288, MESAN2004575, MESAN2005284, MESAN2005303, MESAN2005371, MESAN2005633, MESAN2005689, MESAN2005724, MESAN2005808, MESAN2005811, MESAN2005957, MESAN2006022, MESAN2006043, MESAN2006328, MESAN2006401, MESAN2006563, MESAN2006580, MESAN2006599, MESAN2006743, MESAN2006953, MESAN2006971, MESAN2007032, MESAN2007440, MESAN2007597, MESAN2007755, MESAN2008222, MESAN2008334, MESAN2008415, MESAN2008460, MESAN2008536, MESAN2008679, MESAN2008729, MESAN2008821, MESAN2008926,

MESAN2008936, MESAN2009019, MESAN2009081, MESAN2009105, MESAN2009311, MESAN2009418, MESAN2009503, MESAN2009522, MESAN2010031, MESAN2010114, MESAN2010312, MESAN2010321, MESAN2010664, MESAN2010681, MESAN2011545, MESAN2011597, MESAN2011627, MESAN2011632, MESAN2011977, MESAN2011984, MESAN2012054, MESAN2012113, MESAN2012586, MESAN2012708, MESAN2013019, MESAN2013022, MESAN2013183, MESAN2013211, MESAN2013283, MESAN2013284, MESAN2013611, MESAN2013674, MESAN2013845, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016552,

MESAN2016965, MESAN2017133, MESAN2017152, MESAN2017373, MESAN2017417, MESAN2018209, MESAN2018576, MESAN2018670, MESAN2018699, MESTC1000042, MESTC1000147, MESTC2000150, MESTC2000153, MESTC2000170, N1ESE2000698, NB9N41000011, NB9N41000047, NB9N41000135, NB9N41000142, NB9N41000146, NB9N42000122, NB9N42000196, NB9N42000281, NB9N42000314, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182,

NETRP2000439, NHNPC1000034, NHNPC1000037, NHNPC1000084, NHNPC1000101, NHNPC1000124, NHNPC2000062, NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000091, NOVAR1000102, NOVAR2000038, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000004, NT2NE1000014, NT2NE1000018, NT2NE1000023, NT2NE1000024, NT2NE1000063, NT2NE1000073, NT2NE1000083, NT2NE1000120, NT2NE1000122, NT2NE1000163, NT2NE1000175, NT2NE1000181, NT2NE1000185, NT2NE2000038, NT2NE2000054, NT2NE2000057, NT2NE2000064, NT2NE2000109,

NT2NE2000137, NT2NE2000156, NT2NE2000195, NT2NE2000222, NT2NE2000259, NT2NE2000284, NT2NE2000299, NT2NE2000327, NT2NE2000353, NT2NE2000356, NT2NE2000369, NT2NE2000374, NT2NE2000384, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000586, NT2NE2000612, NT2NE2000658, NT2NE2000706, NT2NE2000763, NT2NE2000802, NT2NE2000808, NT2NE2000809, NT2NE2000864, NT2NE2000909, NT2NE2000913, NT2NE2000950, NT2NE2000951, NT2NE2000980, NT2NE2001005, NT2NE2001021, NT2NE2001040, NT2NE2001048, NT2NE2001049, NT2NE2001142, NT2NE2001156, NT2NE2001176, NT2NE2001180, NT2NE2001181, NT2NE2001188, NT2NE2001191,

NT2NE2001247, NT2NE2001252′, NT2NE2001324, NT2NE2001326, NT2NE2001337, NT2NE2001364, NT2NE2001372, NT2NE2001397, NT2NE2001403, NT2NE2001428, NT2NE2001432, NT2NE2001435, NT2NE2001442, NT2NE2001524, NT2NE2001530, NT2NE2001545, NT2NE2001598, NT2NE2001617, NT2NE2001623, NT2NE2001626, NT2NE2001634, NT2NE2001648, NT2NE2001660, NT2NE2001666, NT2NE2001677, NT2NE2001697, NT2NE2001698, NT2NE2001723, NT2NE2001725, NT2NE2001793, NT2NE2001874, NT2NE2001889, NT2NE2001923, NT2NE2002100, NT2NE2002162, NT2NE2002186, NT2NE2002285, NT2NE2002311, NT2NE2002651, NT2NE2002732, NT2NE2002768, NT2NE2002791, NT2NE2002856, NT2NE2002870, NT2NE2003150, NT2NE2003185, NT2NE2003252, NT2NE2003280, NT2NE2003308, NT2NE2003309,

NT2NE2003315, NT2NE2003393, NT2NE2003408, NT2NE2003457, NT2NE2003485, NT2NE2003540, NT2NE2003571, NT2NE2003705, NT2NE2003752, NT2NE2003887, NT2NE2004255, NT2NE2004378, NT2NE2004490, NT2NE2004519, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005419, NT2NE2005441, NT2NE2005517, NT2NE2005537, NT2NE2005555, NT2NE2005588, NT2NE2005676, NT2NE2005720, NT2NE2005784, NT2NE2005804, NT2NE2005821, NT2NE2005876, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427,

NT2NE2006432, NT2NE2006478, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010842, NT2NE2010854, NT2NE2011033,

NT2NE2011036, NT2NE2011107, NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014104, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275,

NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018273, NT2NE2018376, NT2NE2018472, NT2NE2018490, NT2NE2018594, NT2NE2018739, NT2NE2018916, NT2NE2019056, NT2NE2019076, NT2NE2019099, NT2RI1000016, NT2RI1000035, NT2RI1000048, NT2RI1000164, NT2RI1000172, NT2RI2000004, NT2RI2000007,

NT2RI2000107, NT2RI2000116, NT2RI2000117, NT2RI2000128, NT2RI2000133, NT2RI2000167, NT2RI2000173, NT2RI2000187, NT2RI2000255, NT2RI2000263, NT2RI2000270, NT2RI2000276, NT2RI2000294, NT2RI2000313, NT2RI2000341, NT2RI2000344, NT2RI2000348, NT2RI2000412, NT2RI2000421, NT2RI2000480, NT2RI2000575, NT2RI2000578, NT2RI2000588, NT2RI2000597, NT2RI2000685, NT2RI2000688, NT2RI2000689, NT2RI2000704, NT2RI2000738, NT2RI2000775, NT2RI2000822, NT2RI2000829, NT2RI2000841, NT2RI2000865, NT2RI2000932, NT2RI2000987, NT2RI2001010, NT2RI2001017, NT2RI2001030, NT2RI2001054, NT2RI2001083, NT2RI2001091, NT2RI2001167, NT2RI2001178, NT2RI2001230, NT2RI2001235, NT2RI2001244, NT2RI2001307, NT2RI2001342, NT2RI2001385,

NT2RI2001409, NT2RI2001410, NT2RI2001422, NT2RI2001425, NT2RI2001449, NT2RI2001450, NT2RI2001474, NT2RI2001519, NT2RI2001595, NT2RI2001621, NT2RI2001624, NT2RI2001726, NT2RI2001769, NT2RI2001782, NT2RI2001836, NT2RI2001846, NT2RI2001866, NT2RI2001910, NT2RI2001952, NT2RI2002022, NT2RI2002041, NT2RI2002117, NT2RI2002120, NT2RI2002152, NT2RI2002153, NT2RI2002179, NT2RI2002243, NT2RI2002252, NT2RI2002260, NT2RI2002266, NT2RI2002270, NT2RI2002316, NT2RI2002334, NT2RI2002359, NT2RI2002447, NT2RI2002481, NT2RI2002507, NT2RI2002517, NT2RI2002530, NT2RI2002540, NT2RI2002549, NT2RI2002554, NT2RI2002564, NT2RI2002585, NT2RI2002592, NT2RI2002602, NT2RI2002699, NT2RI2002729, NT2RI2002802, NT2RI2002819,

NT2RI2002822, NT2RI2002847, NT2RI2002852, NT2RI2002865, NT2RI2002923, NT2RI2002926, NT2RI2002958, NT2RI2002970, NT2RI2003011, NT2RI2003019, NT2RI2003051, NT2RI2003067, NT2RI2003184, NT2RI2003205, NT2RI2003222, NT2RI2003301, NT2RI2003317, NT2RI2003344, NT2RI2003360, NT2RI2003361, NT2RI2003383, NT2RI2003392, NT2RI2003399, NT2RI2003419, NT2RI2003487, NT2RI2003556, NT2RI2003655, NT2RI2003667, NT2RI2003678, NT2RI2003884, NT2RI2003973, NT2RI2004078, NT2RI2004079, NT2RI2004093, NT2RI2004136, NT2RI2004157, NT2RI2004188, NT2RI2004190, NT2RI2004209, NT2RI2004230, NT2RI2004284, NT2RI2004290, NT2RI2004304, NT2RI2004312, NT2RI2004381, NT2RI2004398, NT2RI2004410, NT2RI2004442, NT2RI2004468, NT2RI2004606,

NT2RI2004608, NT2RI2004618, NT2RI2004783, NT2RI2004818, NT2RI2004840, NT2RI2004884, NT2RI2004916, NT2RI2004962, NT2RI2004984, NT2RI2004985, NT2RI2005048, NT2RI2005061, NT2RI2005069, NT2RI2005096, NT2RI2005115, NT2RI2005116, NT2RI2005130, NT2RI2005166, NT2RI2005239, NT2RI2005246, NT2RI2005315, NT2RI2005353, NT2RI2005358, NT2RI2005368, NT2RI2005382, NT2RI2005473, NT2RI2005520, NT2RI2005564, NT2RI2005579, NT2RI2005621, NT2RI2005628, NT2RI2005647, NT2RI2005670, NT2RI2005710, NT2RI2005713, NT2RI2005772, NT2RI2005811, NT2RI2005814, NT2RI2005816, NT2RI2005818, NT2RI2005966, NT2RI2006070, NT2RI2006071, NT2RI2006072, NT2RI2006081, NT2RI2006126, NT2RI2006127, NT2RI2006183, NT2RI2006257, NT2RI2006271,

NT2RI2006294, NT2RI2006345, NT2RI2006403, NT2RI2006445, NT2RI2006487, NT2RI2006506, NT2RI2006553, NT2RI2006565, NT2RI2006640, NT2RI2006647, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006838, NT2RI2006853, NT2RI2006855, NT2RI2006943, NT2RI2006973, NT2RI2007046, NT2RI2007048, NT2RI2007084, NT2RI2007096, NT2RI2007116, NT2RI2007214, NT2RI2007277, NT2RI2007303, NT2RI2007334, NT2RI2007377, NT2RI2007386, NT2RI2007406, NT2RI2007439, NT2RI2007445, NT2RI2007468, NT2RI2007469, NT2RI2007498, NT2RI2007507, NT2RI2007572, NT2RI2007589, NT2RI2007593, NT2RI2007629, NT2RI2007723, NT2RI2007729,

NT2RI2007751, NT2RI2007827, NT2RI2007853, NT2RI2007877, NT2RI2007879, NT2RI2007884, NT2RI2007891, NT2RI2007956, NT2RI2007965, NT2RI2008007, NT2RI2008050, NT2RI2008188, NT2RI2008204, NT2RI2008221, NT2RI2008233, NT2RI2008336, NT2RI2008455, NT2RI2008502, NT2RI2008566, NT2RI2008656, NT2RI2008714, NT2RI2008724, NT2RI2008749, NT2RI2008801, NT2RI2008808, NT2RI2008812, NT2RI2008841, NT2RI2008942, NT2RI2008952, NT2RI2009005, NT2RI2009066, NT2RI2009083, NT2RI2009101, NT2RI2009103, NT2RI2009144, NT2RI2009151, NT2RI2009173, NT2RI2009215, NT2RI2009239, NT2RI2009259, NT2RI2009281, NT2RI2009289, NT2RI2009301, NT2RI2009402, NT2RI2009406, NT2RI2009517, NT2RI2009585, NT2RI2009595, NT2RI2009855, NT2RI2010283,

NT2RI2010508, NT2RI2010795, NT2RI2011422, NT2RI2011450, NT2RI2011683, NT2RI2011803, NT2RI2012350, NT2RI2012659, NT2RI2012726, NT2RI2012990, NT2RI2013345, NT2RI2013357, NT2RI2013373, NT2RI2014247, NT2RI2014551, NT2RI2014683, NT2RI2014733, NT2RI2015239, NT2RI2015342, NT2RI2015533, NT2RI2016128, NT2RI2016134, NT2RI2017529, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758,

NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006284,

NT2RI3006340, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000005, NT2RP6000008, NT2RP6000017, NT2RP6000032, NT2RP6000043, NT2RP6000059, NT2RP6000060, NT2RP6000072, NT2RP6000077, NT2RP6000078, NT2RP6000085, NT2RP6000086, NT2RP6000100, NT2RP6000109, NT2RP6000110, NT2RP6000119, NT2RP6000127, NT2RP6000130, NT2RP6000140, NT2RP6000150, NT2RP7000041, NT2RP7000069, NT2RP7000086,

NT2RP7000109, NT2RP7000112, NT2RP7000173, NT2RP7000238, NT2RP7000259, NT2RP7000271, NT2RP7000311, NT2RP7000359, NT2RP7000364, NT2RP7000380, NT2RP7000391, NT2RP7000425, NT2RP7000477, NT2RP7000579, NT2RP7000586, NT2RP7000600, NT2RP7000624, NT2RP7000812, NT2RP7000906, NT2RP7000926, NT2RP7001074, NT2RP7001080, NT2RP7001156, NT2RP7001231, NT2RP7001306, NT2RP7001319, NT2RP7001335, NT2RP7001364, NT2RP7001475, NT2RP7001532, NT2RP7001602, NT2RP7001678, NT2RP7001856, NT2RP7002064, NT2RP7002151, NT2RP7002163, NT2RP7002243, NT2RP7002282, NT2RP7002376, NT2RP7002478, NT2RP7002554, NT2RP7002594, NT2RP7002603, NT2RP7002650, NT2RP7002738, NT2RP7002779, NT2RP7002802, NT2RP7002829, NT2RP7002841, NT2RP7002875,

NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003203, NT2RP7003261, NT2RP7003319, NT2RP7003439, NT2RP7003511, NT2RP7003629, NT2RP7003632, NT2RP7003647, NT2RP7003680, NT2RP7003688, NT2RP7003724, NT2RP7004027, NT2RP7004080, NT2RP7004114, NT2RP7004123, NT2RP7004173, NT2RP7004196, NT2RP7004204, NT2RP7004233, NT2RP7004260, NT2RP7004352, NT2RP7004358, NT2RP7004373, NT2RP7004396, NT2RP7004428, NT2RP7004481, NT2RP7004541, NT2RP7004559, NT2RP7004641, NT2RP7004656, NT2RP7004687, NT2RP7004722, NT2RP7004728, NT2RP7004766, NT2RP7004790, NT2RP7004884, NT2RP7004911, NT2RP7004915, NT2RP7004946,

NT2RP7004961, NT2RP7004964, NT2RP7004975, NT2RP7005118, NT2RP7005205, NT2RP7005219, NT2RP7005323, NT2RP7005468, NT2RP7005493, NT2RP7005513, NT2RP7005529, NT2RP7005629, NT2RP7005631, NT2RP7005669, NT2RP7005675, NT2RP7005750, NT2RP7005846, NT2RP7006033, NT2RP7006141, NT2RP7006160, NT2RP7006162, NT2RP7006188, NT2RP7006223, NT2RP7006263, NT2RP7006296, NT2RP7006304, NT2RP7006374, NT2RP7006395, NT2RP7006408, NT2RP7006457, NT2RP7006490, NT2RP7006539, NT2RP7006547, NT2RP7006601, NT2RP7006619, NT2RP7006621, NT2RP7006636, NT2RP7006701, NT2RP7006853, NT2RP7006886, NT2RP7006887, NT2RP7006980, NT2RP7006986, NT2RP7006995, NT2RP7007114, NT2RP7007221, NT2RP7007226, NT2RP7007252, NT2RP7007269, NT2RP7007310,

NT2RP7007359, NT2RP7007381, NT2RP7007387, NT2RP7007406, NT2RP7007480, NT2RP7007504, NT2RP7007530, NT2RP7007537, NT2RP7007580, NT2RP7007594, NT2RP7007610, NT2RP7007617, NT2RP7007643, NT2RP7007766, NT2RP7007842, NT2RP7007925, NT2RP7007930, NT2RP7007975, NT2RP7008013, NT2RP7008015, NT2RP7008133, NT2RP7008137, NT2RP7008142, NT2RP7008144, NT2RP7008161, NT2RP7008167, NT2RP7008190, NT2RP7008315, NT2RP7008360, NT2RP7008396, NT2RP7008406, NT2RP7008435, NT2RP7008441, NT2RP7008454, NT2RP7008487, NT2RP7008543, NT2RP7008544, NT2RP7008550, NT2RP7008557, NT2RP7008657, NT2RP7008714, NT2RP7008720, NT2RP7008855, NT2RP7008863, NT2RP7009012, NT2RP7009019, NT2RP7009030, NT2RP7009097, NT2RP7009108, NT2RP7009147,

NT2RP7009168, NT2RP7009215, NT2RP7009225, NT2RP7009236, NT2RP7009253, NT2RP7009259, NT2RP7009267, NT2RP7009322, NT2RP7009363, NT2RP7009370, NT2RP7009373, NT2RP7009394, NT2RP7009397, NT2RP7009429, NT2RP7009439, NT2RP7009466, NT2RP7009481, NT2RP7009482, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7013002, NT2RP7013374, NT2RP7013499, NT2RP7013573, NT2RP7013729, NT2RP7013764, NT2RP7013795, NT2RP7013999,

NT2RP7014178, NT2RP7014348, NT2RP7014420, NT2RP7014583, NT2RP7014721, NT2RP7014743, NT2RP7014778, NT2RP7014906, NT2RP7014910, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, NT2RP7019835, NT2RP7020112, NT2RP7020123, NT2RP7020343, NT2RP7020379, NT2RP8000137,

NT2RP8000296, NT2RP8000435, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8008057, NT2RP8009248, NTONG1000006, NTONG1000033, NTONG1000047, NTONG1000052, NTONG1000075, NTONG1000087, NTONG1000098, NTONG1000123, NTONG1000130, NTONG1000161, NTONG1000172, NTONG1000213, NTONG1000214, NTONG1000246, NTONG1000247, NTONG1000264, NTONG2000040, NTONG2000107, NTONG2000231, NTONG2000334, NTONG2000363, NTONG2000413, NTONG2000492, NTONG2000499, NTONG2000531, NTONG2000583, NTONG2000800, NTONG2000858,

NTONG2000876, NTONG2000966, NTONG2000977, NTONG2000985, NTONG2001014, NTONG2001079, NTONG2001137, NTONG2001157, NTONG2001173, NTONG2001220, NTONG2001222, NTONG2001362, NTONG2001428, NTONG2001532, NTONG2001550, NTONG2001587, NTONG2001612, NTONG2001619, NTONG2001642, NTONG2001657, NTONG2001676, NTONG2002278, NTONG2002582, NTONG2002807, NTONG2002970, NTONG2002985, NTONG2003158, NTONG2003210, NTONG2003316, NTONG2003409, NTONG2003454, NTONG2003515, NTONG2003805, NTONG2003852, NTONG2003897, NTONG2003928, NTONG2004095, NTONG2004308, NTONG2004521, NTONG2004614, NTONG2004690, NTONG2004806, NTONG2004829, NTONG2004844, NTONG2004918, NTONG2004991, NTONG2005062, NTONG2005086, NTONG2005137, NTONG2005153,

NTONG2005265, NTONG2005277, NTONG2005363, NTONG2005373, NTONG2005391, NTONG2005480, NTONG2005497, NTONG2005520, NTONG2005577, NTONG2005657, NTONG2005801, NTONG2005822, NTONG2005897, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007693, NTONG2007756, NTONG2008088, NTONG2008093, NTONG2008143, NTONG2008365, NTONG2008522, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030,

OCBBF1000042, OCBBF1000049, OCBBF1000054, OCBBF1000067, OCBBF1000080, OCBBF1000085, OCBBF1000091, OCBBF1000104, OCBBF1000114, OCBBF1000118, OCBBF1000119, OCBBF1000122, OCBBF1000130, OCBBF1000138, OCBBF1000145, OCBBF1000146, OCBBF1000153, OCBBF1000185, OCBBF1000188, OCBBF1000192, OCBBF1000254, OCBBF1000315, OCBBF2000013, OCBBF2000015, OCBBF2000126, OCBBF2000178, OCBBF2000231, OCBBF2000277, OCBBF2000287, OCBBF2000323, OCBBF2000452, OCBBF2000467, OCBBF2000522, OCBBF2000677, OCBBF2000703, OCBBF2000719, OCBBF2000824, OCBBF2000831, OCBBF2000846, OCBBF2000904, OCBBF2000986, OCBBF2000998, OCBBF2001075, OCBBF2001101, OCBBF2001124, OCBBF2001140, OCBBF2001166, OCBBF2001176, OCBBF2001186, OCBBF2001196,

OCBBF2001210, OCBBF2001252, OCBBF2001307, OCBBF2001323, OCBBF2001389, OCBBF2001402, OCBBF2001408, OCBBF2001416, OCBBF2001473, OCBBF2001494, OCBBF2001527, OCBBF2001528, OCBBF2001586, OCBBF2001639, OCBBF2001681, OCBBF2001687, OCBBF2001706, OCBBF2001749, OCBBF2001794, OCBBF2001910, OCBBF2001931, OCBBF2001961, OCBBF2001983, OCBBF2002015, OCBBF2002083, OCBBF2002086, OCBBF2002124, OCBBF2002161, OCBBF2002290, OCBBF2002357, OCBBF2002376, OCBBF2002615, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003518, OCBBF2003543, OCBBF2003593, OCBBF2003744, OCBBF2003819, OCBBF2003925, OCBBF2004038,

OCBBF2004104, OCBBF2004147, OCBBF2004168, OCBBF2004235, OCBBF2004259, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843, OCBBF2005901, OCBBF2005956, OCBBF2006005, OCBBF2006030, OCBBF2006058, OCBBF2006113, OCBBF2006148, OCBBF2006151, OCBBF2006154, OCBBF2006172, OCBBF2006214, OCBBF2006241, OCBBF2006313, OCBBF2006388, OCBBF2006438, OCBBF2006567, OCBBF2006624,

OCBBF2006639, OCBBF2006660, OCBBF2006764, OCBBF2006849, OCBBF2006859, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007354, OCBBF2007414, OCBBF2007415, OCBBF2007428, OCBBF2007478, OCBBF2007485, OCBBF2007520, OCBBF2007555, OCBBF2007610, OCBBF2007622, OCBBF2007756, OCBBF2007762, OCBBF2007892, OCBBF2007931, OCBBF2007946, OCBBF2008005, OCBBF2008041, OCBBF2008116, OCBBF2008138, OCBBF2008283, OCBBF2008330, OCBBF2008340, OCBBF2008366, OCBBF2008466, OCBBF2008511, OCBBF2008520, OCBBF2008569, OCBBF2008586, OCBBF2008640, OCBBF2008691, OCBBF2008701, OCBBF2008724,

OCBBF2008760, OCBBF2008768, OCBBF2008770, OCBBF2008775, OCBBF2008790, OCBBF2008814, OCBBF2008822, OCBBF2009095, OCBBF2009115, OCBBF2009242, OCBBF2009301, OCBBF2009352, OCBBF2009391, OCBBF2009536, OCBBF2009571, OCBBF2009583, OCBBF2009603, OCBBF2009772, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010841, OCBBF2010843, OCBBF2010858, OCBBF2010863, OCBBF2010871, OCBBF2010931, OCBBF2010945, OCBBF2010978, OCBBF2010989, OCBBF2011073, OCBBF2011137, OCBBF2011160, OCBBF2011177, OCBBF2011228, OCBBF2011232, OCBBF2011283,

OCBBF2011311, OCBBF2011625, OCBBF2011669, OCBBF2011685, OCBBF2011706, OCBBF2011722, OCBBF2011759, OCBBF2011767, OCBBF2011855, OCBBF2011872, OCBBF2011887, OCBBF2011897, OCBBF2011914, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012553, OCBBF2012678, OCBBF2012704, OCBBF2012755, OCBBF2012812, OCBBF2012821, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789,

OCBBF2013843, OCBBF2013883, OCBBF2013926, OCBBF2014052, OCBBF2014064, OCBBF2014089, OCBBF2014322, OCBBF2014386, OCBBF2014541, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016841, OCBBF2016928, OCBBF2017035, OCBBF2017055, OCBBF2017069, OCBBF2017306, OCBBF2017325, OCBBF2017398, OCBBF2017458,

OCBBF2017489, OCBBF2017516, OCBBF2017665, OCBBF2017754, OCBBF2017791, OCBBF2017815, OCBBF2017882, OCBBF2017888, OCBBF2017893, OCBBF2017920, OCBBF2018012, OCBBF2018014, OCBBF2018084, OCBBF2018167, OCBBF2018206, OCBBF2018229, OCBBF2018234, OCBBF2018362, OCBBF2018380, OCBBF2018563, OCBBF2018581, OCBBF2018618, OCBBF2018663, OCBBF2018687, OCBBF2018827, OCBBF2018828, OCBBF2018873, OCBBF2018934, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021286, OCBBF2021323, OCBBF2021518, OCBBF2021788, OCBBF2021833,

OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927,

OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000323, OCBBF3000372, OCBBF3000380,

OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004487, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000174, PEBLM1000180, PEBLM2000030,

PEBLM2000112, PEBLM2000126, PEBLM2000147, PEBLM2000170, PEBLM2000180, PEBLM2000213, PEBLM2000222, PEBLM2000248, PEBLM2000267, PEBLM2000270, PEBLM2000308, PEBLM2000321, PEBLM2000326, PEBLM2000333, PEBLM2000338, PEBLM2000395, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004452, PEBLM2004497, PEBLM2004666, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283,

PEBLM2006298, PEBLM2006912, PEBLM2007112, PEBLM2007140, PEBLM2007188, PEBLM2007296, PEBLM2007437, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131,

PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007068, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000066, PLACE5000067, PLACE5000068, PLACE5000070, PLACE5000105, PLACE5000113, PLACE5000115, PLACE5000116, PLACE5000129, PLACE5000139, PLACE5000153, PLACE5000159, PLACE5000171, PLACE5000172, PLACE5000178, PLACE5000245, PLACE5000260, PLACE5000297, PLACE5000372, PLACE5000492,

PLACE5000522, PLACE5000527, PLACE6000012, PLACE6000044, PLACE6000055, PLACE6000070, PLACE6000137, PLACE6000145, PLACE6000191, PLACE6000205, PLACE6000231, PLACE6000263, PLACE6000296, PLACE6000314, PLACE6000348, PLACE6000371, PLACE6000424, PLACE6000426, PLACE6000429, PLACE6000450, PLACE6000463, PLACE6000523, PLACE6000550, PLACE6000552, PLACE6000555, PLACE6000619, PLACE6000630, PLACE6000953, PLACE6001118, PLACE6001153, PLACE6001185, PLACE6001262, PLACE6001294, PLACE6001443, PLACE6001536, PLACE6001557, PLACE6001712, PLACE6001740, PLACE6001823, PLACE6001886, PLACE6001923, PLACE6001925, PLACE6001933, PLACE6002011, PLACE6002016, PLACE6002056, PLACE6002102, PLACE6002157, PLACE6002312, PLACE6002323,

PLACE6002345, PLACE6002419, PLACE6002462, PLACE6002647, PLACE6002668, PLACE6002692, PLACE6002699, PLACE6002710, PLACE6002871, PLACE6002949, PLACE6002960, PLACE6003004, PLACE6003038, PLACE6003077, PLACE6003094, PLACE6003109, PLACE6003204, PLACE6003218, PLACE6003372, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004005, PLACE6004101, PLACE6004219, PLACE6004336, PLACE6004368, PLACE6004380, PLACE6004396, PLACE6004397, PLACE6004454, PLACE6004464, PLACE6004491, PLACE6004663, PLACE6004687, PLACE6004738, PLACE6004931, PLACE6004966, PLACE6004993, PLACE6005007, PLACE6005029, PLACE6005108,

PLACE6005231, PLACE6005234, PLACE6005283, PLACE6005294, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006077, PLACE6006137, PLACE6006158, PLACE6006217, PLACE6006222, PLACE6006266, PLACE6006287, PLACE6006394, PLACE6006418, PLACE6006474, PLACE6006549, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007482, PLACE6007687, PLACE6007787, PLACE6007798, PLACE6007807, PLACE6007925, PLACE6008036, PLACE6008094, PLACE6008126, PLACE6008285,

PLACE6008444, PLACE6008453, PLACE6008640, PLACE6008750, PLACE6008768, PLACE6008775, PLACE6008793, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010077, PLACE6010463, PLACE6010485, PLACE6010568, PLACE6010701, PLACE6010704, PLACE6010732, PLACE6010765, PLACE6010925, PLACE6010936, PLACE6010991, PLACE6011057, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012108, PLACE6012168, PLACE6012297,

PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016254, PLACE6016942, PLACE6016984, PLACE6017002, PLACE6017431, PLACE6017564, PLACE6017626, PLACE6017701, PLACE6017714, PLACE6017788, PLACE6018031, PLACE6018107, PLACE6018187, PLACE6018235, PLACE6018287, PLACE6018441, PLACE6018497, PLACE6018769,

PLACE6018834, PLACE6018843, PLACE6018863, PLACE6018938, PLACE6019195, PLACE6019542, PLACE6019600, PLACE6019674, PLACE6019676, PLACE6019701, PLACE6019932, PLACE6020031, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004103, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563,

PROST1000033, PROST1000039, PROST1000065, PROST1000083, PROST1000085, PROST1000110, PROST1000120, PROST1000123, PROST1000136, PROST1000144, PROST1000145, PROST1000152, PROST1000167, PROST1000184, PROST1000215, PROST1000217, PROST1000220, PROST1000226, PROST1000246, PROST1000262, PROST1000272, PROST1000298, PROST1000322, PROST1000334, PROST1000362, PROST1000377, PROST1000383, PROST1000412, PROST1000419, PROST1000451, PROST1000480, PROST1000485, PROST1000493, PROST1000526, PROST1000527, PROST1000528, PROST1000559, PROST1000575, PROST2000036, PROST2000053, PROST2000067, PROST2000079, PROST2000105, PROST2000143, PROST2000162, PROST2000165, PROST2000176, PROST2000203, PROST2000206, PROST2000212,

PROST2000241, PROST2000267, PROST2000273, PROST2000274, PROST2000277, PROST2000284, PROST2000325, PROST2000337, PROST2000432, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002, PROST2001116, PROST2001138, PROST2001180, PROST2001213, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001823, PROST2001883, PROST2001899, PROST2001997, PROST2001998, PROST2002101, PROST2002162, PROST2002212, PROST2002227, PROST2002338, PROST2002425, PROST2002488,

PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003232, PROST2003302, PROST2003303, PROST2003324, PROST2003340, PROST2003396, PROST2003428, PROST2003472, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004258, PROST2004270, PROST2004332, PROST2004481, PROST2004526,

PROST2004570, PROST2004727, PROST2004739, PROST2004742, PROST2004744, PROST2004817, PROST2005039, PROST2005067, PROST2005076, PROST2005093, PROST2005121, PROST2005131, PROST2005143, PROST2005228, PROST2005272, PROST2005285, PROST2005426, PROST2005466, PROST2005604, PROST2005630, PROST2005683, PROST2005788, PROST2005793, PROST2005880, PROST2005886, PROST2005904, PROST2005919, PROST2005943, PROST2006008, PROST2006020, PROST2006030, PROST2006060, PROST2006196, PROST2006201, PROST2006260, PROST2006282, PROST2006343, PROST2006450, PROST2006491, PROST2006510, PROST2006536, PROST2006579, PROST2006688, PROST2006737, PROST2006766, PROST2006782, PROST2006933, PROST2006988, PROST2007122, PROST2007200,

PROST2007237, PROST2007248, PROST2007289, PROST2007317, PROST2007328, PROST2007382, PROST2007389, PROST2007416, PROST2007528, PROST2007612, PROST2007818, PROST2007871, PROST2007950, PROST2007956, PROST2007974, PROST2007988, PROST2008079, PROST2008088, PROST2008243, PROST2008245, PROST2008268, PROST2008271, PROST2008360, PROST2008447, PROST2008468, PROST2008472, PROST2008516, PROST2008724, PROST2008770, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046,

PROST2010219, PROST2010250, PROST2010318, PROST2010326, PROST2010382, PROST2010400, PROST2010545, PROST2010579, PROST2010606, PROST2010885, PROST2011012, PROST2011038, PROST2011105, PROST2011297, PROST2011410, PROST2011439, PROST2011482, PROST2011577, PROST2011631, PROST2011660, PROST2012005, PROST2012007, PROST2012016, PROST2012088, PROST2012140, PROST2012157, PROST2012190, PROST2012249, PROST2012353, PROST2012400, PROST2012448, PROST2012504, PROST2012542, PROST2012550, PROST2012740, PROST2012888, PROST2012890, PROST2013032, PROST2013121, PROST2013260, PROST2013327, PROST2013531, PROST2013605, PROST2013627, PROST2013873, PROST2013880, PROST2014422, PROST2014585, PROST2014601, PROST2014659,

PROST2014862, PROST2014916, PROST2014925, PROST2015124, PROST2015137, PROST2015198, PROST2015243, PROST2015246, PROST2015251, PROST2015287, PROST2015332, PROST2015457, PROST2015464, PROST2015537, PROST2015545, PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2016980, PROST2017098, PROST2017128, PROST2017367, PROST2017413, PROST2017441, PROST2017529, PROST2017578, PROST2017612, PROST2017617, PROST2017700,

PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019164, PROST2019253, PROST2019296, PROST2019398, PROST2019487, PROST2019781, PUAEN1000039, PUAEN1000057, PUAEN1000065, PUAEN1000081, PUAEN1000085, PUAEN1000087, PUAEN1000121, PUAEN1000147, PUAEN1000161, PUAEN1000164, PUAEN1000175, PUAEN1000239, PUAEN1000275, PUAEN1000276, PUAEN1000288, PUAEN1000322, PUAEN2000080, PUAEN2000152, PUAEN2000175, PUAEN2000247, PUAEN2000312, PUAEN2000394, PUAEN2000535, PUAEN2000594, PUAEN2000684, PUAEN2000942,

PUAEN2001260, PUAEN2001345, PUAEN2001526, PUAEN2001586, PUAEN2001882, PUAEN2002473, PUAEN2002489, PUAEN2002552, PUAEN2002568, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007044, PUAEN2007350, PUAEN2007785, PUAEN2007898, PUAEN2008123, PUAEN2008228, PUAEN2008314, PUAEN2008515, PUAEN2008815, PUAEN2008939, PUAEN2008980, PUAEN2008985, PUAEN2009174, PUAEN2009348, PUAEN2009534, PUAEN2009655,

PUAEN2009795, PUAEN2009852, PUAEN2010346, PUAEN2010824, RECTM1000051, RECTM1000141, RECTM2000220, RECTM2000349, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000047, SALGL1000065, SALGL1000107, SALGL1000171, SKMUS1000014, SKMUS1000022, SKMUS1000030, SKMUS1000067, SKMUS1000102, SKMUS1000104, SKMUS1000110, SKMUS1000118, SKMUS1000124, SKMUS1000129, SKMUS1000176, SKMUS1000177, SKMUS2000020, SKMUS2000061, SKMUS2000074, SKMUS2000078, SKMUS2000117, SKMUS2000137, SKMUS2000243, SKMUS2000271, SKMUS2000287, SKMUS2000317, SKMUS2000330, SKMUS2000339,

SKMUS2000343, SKMUS2000365, SKMUS2000390, SKMUS2000416, SKMUS2000467, SKMUS2000468, SKMUS2000484, SKMUS2000515, SKMUS2000690, SKMUS2000701, SKMUS2000705, SKMUS2000710, SKMUS2000724, SKMUS2000726, SKMUS2000774, SKMUS2000780, SKMUS2000847, SKMUS2000863, SKMUS2000894, SKMUS2000898, SKMUS2000902, SKMUS2000933, SKMUS2000945, SKMUS2000954, SKMUS2000961, SKMUS2000973, SKMUS2000980, SKMUS2001014, SKMUS2001129, SKMUS2001147, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001454, SKMUS2001492, SKMUS2001528, SKMUS2001543, SKMUS2001580, SKMUS2001585,

SKMUS2001608, SKMUS2001631, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607,

SKMUS2009190, SKMUS2009232, SKMUS2009479, SKMUS2009557, SKNMC1000110, SKNMC1000123, SKNMC1000124, SKNMC1000159, SKNMC1000229, SKNMC1000251, SKNMC1000264, SKNMC2000065, SKNMC2000097, SKNMC2000256, SKNMC2000305, SKNMC2000322, SKNMC2000356, SKNMC2000374, SKNMC2000583, SKNMC2000593, SKNMC2000612, SKNMC2000622, SKNMC2000877, SKNMC2000966, SKNMC2001113, SKNMC2001324, SKNMC2001503, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000002, SKNSH1000086, SKNSH1000101, SKNSH1000174,

SKNSH1000301, SKNSH1000308, SKNSH1000416, SKNSH2000051, SKNSH2000101, SKNSH2000151, SKNSH2000163, SKNSH2000250, SKNSH2000319, SKNSH2000347, SKNSH2000482, SKNSH2000516, SKNSH2000550, SKNSH2000819, SKNSH2000971, SKNSH2001222, SKNSH2001875, SKNSH2002054, SKNSH2002325, SKNSH2002866, SKNSH2003064, SKNSH2003174, SKNSH2003422, SKNSH2003466, SKNSH2003483, SKNSH2003528, SKNSH2003633, SKNSH2004039, SKNSH2005061, SKNSH2005120, SKNSH2005194, SKNSH2005240, SKNSH2005792, SKNSH2005816, SKNSH2005981, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009357, SKNSH2009435, SKNSH2009991, SKNSH2010015,

SMINT1000012, SMINT1000016, SMINT1000042, SMINT1000048, SMINT1000051, SMINT1000054, SMINT1000057, SMINT1000071, SMINT1000075, SMINT1000078, SMINT1000091, SMINT1000100, SMINT1000103, SMINT100011, SMINT1000118, SMINT1000137, SMINT2000007, SMINT2000018, SMINT2000032, SMINT2000040, SMINT2000060, SMINT2000073, SMINT2000084, SMINT2000145, SMINT2000159, SMINT2000176, SMINT2000185, SMINT2000227, SMINT2000229, SMINT2000232, SMINT2000239, SMINT2000267, SMINT2000400, SMINT2000454, SMINT2000518, SMINT2000529, SMINT2000530, SMINT2000537, SMINT2000538, SMINT2000541, SMINT2000545, SMINT2000558, SMINT2000562, SMINT2000568, SMINT2000602, SMINT2000609, SMINT2000629, SMINT2000659, SMINT2000747, SMINT2000811,

SMINT2000824, SMINT2000933, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001950, SMINT2002126, SMINT2002159, SMINT2002202, SMINT2002210, SMINT2002224, SMINT2002281, SMINT2002311, SMINT2002314, SMINT2002328, SMINT2002414, SMINT2002457, SMINT2002620, SMINT2002743, SMINT2002778, SMINT2002880, SMINT2002882, SMINT2002884, SMINT2002899, SMINT2002974, SMINT2002976, SMINT2003003, SMINT2003128, SMINT2003150, SMINT2003317, SMINT2003340, SMINT2003386, SMINT2003423, SMINT2003505, SMINT2003551, SMINT2003569,

SMINT2003641, SMINT2003644, SMINT2003866, SMINT2004023, SMINT2004037, SMINT2004047, SMINT2004086, SMINT2004280, SMINT2004299, SMINT2004339, SMINT2004350, SMINT2004414, SMINT2004473, SMINT2004547, SMINT2004583, SMINT2004589, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005368, SMINT2005387, SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140,

SMINT2007187, SMINT2007219, SMINT2007294, SMINT2007365, SMINT2.007391, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2008921, SMINT2008960, SMINT2009119, SMINT2009121, SMINT2009212, SMINT2009216, SMINT2009233, SMINT2009259, SMINT2009272, SMINT2009292, SMINT2009363, SMINT2009415, SMINT2009468, SMINT2009505, SMINT2009529,

SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010901, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012179, SMINT2012195, SMINT2012285, SMINT2012501, SMINT2012734, SMINT2012793, SMINT2013032, SMINT2013129, SMINT2013170, SMINT2013181, SMINT2013228, SMINT2013613,

SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017659, SMINT2017736, SMINT2017781, SMINT2017855, SMINT2017884, SMINT2017964, SMINT2017974, SMINT2018343, SMINT2018592, SMINT2018681, SMINT2019017, SMINT2019105, SMINT2019142,

SMINT2019153, SMINT2019200, SPLEN1000013, SPLEN1000041, SPLEN1000049, SPLEN1000056, SPLEN1000087, SPLEN1000089, SPLEN1000091, SPLEN1000106, SPLEN1000134, SPLEN1000139, SPLEN1000143, SPLEN1000144, SPLEN1000145, SPLEN1000156, SPLEN1000163, SPLEN1000165, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000210, SPLEN2000222, SPLEN2000242, SPLEN2000243, SPLEN2000247, SPLEN2000255, SPLEN2000267, SPLEN2000270, SPLEN2000307, SPLEN2000310, SPLEN2000341, SPLEN2000350, SPLEN2000357, SPLEN2000364, SPLEN2000402, SPLEN2000414, SPLEN2000420, SPLEN2000443, SPLEN2000448, SPLEN2000450,

SPLEN2000464, SPLEN2000496, SPLEN2000505, SPLEN2000515, SPLEN2000516, SPLEN2000537, SPLEN2000541, SPLEN2000607, SPLEN2000630, SPLEN2000695, SPLEN2000698, SPLEN2000839, SPLEN2001135, SPLEN2001141, SPLEN2001157, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001689, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002335, SPLEN2002343, SPLEN2002354, SPLEN2002385, SPLEN2002419, SPLEN2002451,

SPLEN2002462, SPLEN2002463, SPLEN2002467, SPLEN2002493, SPLEN2002662, SPLEN2002695, SPLEN2002707, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004220, SPLEN2004343, SPLEN2004346, SPLEN2004368, SPLEN2004555, SPLEN2004611, SPLEN2004662, SPLEN2004773, SPLEN2004844, SPLEN2004880, SPLEN2004984, SPLEN2005009, SPLEN2005227, SPLEN2005272, SPLEN2005342, SPLEN2005416, SPLEN2005429, SPLEN2005450, SPLEN2005474, SPLEN2005560, SPLEN2005727, SPLEN2005783, SPLEN2005790, SPLEN2005818, SPLEN2005838, SPLEN2005869,

SPLEN2005939, SPLEN2006122, SPLEN2006133, SPLEN2006143, SPLEN2006232, SPLEN2006268, SPLEN2006283, SPLEN2006305, SPLEN2006374, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007498, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008496, SPLEN2008591, SPLEN2008786, SPLEN2009081, SPLEN2009088, SPLEN2009163, SPLEN2009315, SPLEN2009340, SPLEN2009512, SPLEN2009513, SPLEN2009541, SPLEN2009548, SPLEN2009555,

SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010195, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011419, SPLEN2011422, SPLEN2011672, SPLEN2011678, SPLEN2011737, SPLEN2011758, SPLEN2011766, SPLEN2011805, SPLEN2011820, SPLEN2011830, SPLEN2011931, SPLEN2011981,

SPLEN2012115, SPLEN2012175, SPLEN2012179, SPLEN2012185, SPLEN2012234, SPLEN2012453, SPLEN2012523, SPLEN2012524, SPLEN2012525, SPLEN2012571, SPLEN2012606, SPLEN2012611, SPLEN2012619, SPLEN2012624, SPLEN2012725, SPLEN2012743, SPLEN2012756, SPLEN2012800, SPLEN2012846, SPLEN2012889, SPLEN2012961, SPLEN2013108, SPLEN2013195, SPLEN2013644, SPLEN2013648, SPLEN2013670, SPLEN2013673, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014080, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543,

SPLEN2014645, SPLEN2014669, SPLEN2014711, SPLEN2014739, SPLEN2014860, SPLEN2014865, SPLEN2014911, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015310, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016304, SPLEN2016356, SPLEN2016480, SPLEN2016531, SPLEN2016533,

SPLEN2016541, SPLEN2016552, SPLEN2016554, SPLEN2016602, SPLEN2016627, SPLEN2016678, SPLEN2016773, SPLEN2016781, SPLEN2016840, SPLEN2016863, SPLEN2016922, SPLEN2016932, SPLEN2016973, SPLEN2017043, SPLEN2017104, SPLEN2017121, SPLEN2017147, SPLEN2017187, SPLEN2017189, SPLEN2017212, SPLEN2017318, SPLEN2017351, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017918, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018364, SPLEN2018395, SPLEN2018643, SPLEN2018683, SPLEN2018732, SPLEN2018749, SPLEN2018933,

SPLEN2019008, SPLEN2019043, SPLEN2019077, SPLEN2019092, SPLEN2019102, SPLEN2019131, SPLEN2019169, SPLEN2019257, SPLEN2019311, SPLEN2019323, SPLEN2019349, SPLEN2019375, SPLEN2019379, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021194, SPLEN2021231, SPLEN2021273, SPLEN2021295, SPLEN2021298, SPLEN2021383,

SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027113, SPLEN2027157,

SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028466, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031547, SPLEN2031674, SPLEN2031724, SPLEN2031780, SPLEN2032036, SPLEN2032112, SPLEN2032154, SPLEN2032321, SPLEN2032356, SPLEN2032677, SPLEN2032813, SPLEN2032924, SPLEN2033098,

SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039379, SPLEN2039672, SPLEN2039697, SPLEN2039845,

SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042535, SPLEN2042598, SPLEN2042714, SPLEN2042920, STOMA1000013, STOMA1000047, STOMA1000052, STOMA1000074, STOMA1000091, STOMA1000117, STOMA1000133, STOMA1000175, STOMA1000186, STOMA1000190, STOMA1000191, STOMA2000032, STOMA2000072, STOMA2000088, STOMA2000104, STOMA2000148, STOMA2000168, STOMA2000183, STOMA2000188, STOMA2000257, STOMA2000279, STOMA2000289, STOMA2000383, STOMA2000386, STOMA2000478, STOMA2000482, STOMA2000529, STOMA2000539, STOMA2000567, STOMA2000640, STOMA2000678,

STOMA2000686, STOMA2000888, STOMA2001025, STOMA2001389, STOMA2002052, STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253,

STOMA2009256, STOMA2009289, SYNOV1000049, SYNOV1000118, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001144, SYNOV2001212, SYNOV2001239, SYNOV2001251, SYNOV2001262, SYNOV2001300, SYNOV2001356, SYNOV2001374, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005541, SYNOV2005673,

SYNOV2005817, SYNOV2005962, SYNOV2006430, SYNOV2006620, SYNOV2007758, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001153, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570,

SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007671, SYNOV4007711, SYNOV4008245, SYNOV4008336, SYNOV4009129, SYNOV4009139, SYNOV4009298, SYNOV4009575, T1ESE2000116, T1ESE2000904, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543,

TBAES2006568, TBAES2006881, TBAES2007379, TBAES2007428, TBAES2007481, TBAES2007504, TBAES2007548, TBAES2007697, TBAES2007862, TBAES2007964, TBAES2008133, TBAES2009387, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005,

TESOP2002110, TESOP2002150, TESOP2002273, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247,

TESOP2009555, TESTI1000018, TESTI1000019, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000163, TESTI1000168, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154,

TESTI2000172, TESTI2000177, TESTI2000179, TESTI2000184, TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951,

TESTI2000970, TESTI2000981, TESTI2001008, TESTI2001049, TESTI2001082, TESTI2001120, TESTI2001134, TESTI2001141, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001862, TESTI2001869, TESTI2001879, TESTI2001894,

TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002149, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002365, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002707,

TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003327, TESTI2003330, TESTI2003347,

TESTI2003354, TESTI2003356, TESTI2003372, TESTI2003376, TESTI2003418, TESTI2003419, TESTI2003432, TESTI2003454, TESTI2003475, TESTI2003476, TESTI2003498, TESTI2003512, TESTI2003539, TESTI2003541, TESTI2003545, TESTI2003551, TESTI2003574, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004031, TESTI2004073, TESTI2004080, TESTI2004085, TESTI2004089, TESTI2004111, TESTI2004119, TESTI2004122, TESTI2004153,

TESTI2004163, TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004737, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994,

TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005408, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005743, TESTI2005767, TESTI2005775, TESTI2005784,

TESTI2005788, TESTI2005827, TESTI2005835, TESTI2005860, TESTI2005892, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006453, TESTI2006457, TESTI2006460, TESTI2006465,

TESTI2006483, TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006774, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007220, TESTI2007234, TESTI2007311, TESTI2007320, TESTI2007346, TESTI2007358, TESTI2007402, TESTI2007405, TESTI2007407, TESTI2007422,

TESTI2007452, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2007998, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405,

TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009474, TESTI2009477, TESTI2009497, TESTI2009502, TESTI2009511, TESTI2009520, TESTI2009544, TESTI2009545, TESTI2009551, TESTI2009577,

TESTI2009585, TESTI2009588, TESTI2009683, TESTI2009727, TESTI2009739, TESTI2009785, TESTI2009812, TESTI2009835, TESTI2009853, TESTI2009935, TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011294, TESTI2011314, TESTI2011315, TESTI2011394, TESTI2011407, TESTI2011448, TESTI2011480, TESTI2011549, TESTI2011575, TESTI2011605, TESTI2011612,

TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012155, TESTI2012171, TESTI2012207, TESTI2012231, TESTI2012244, TESTI2012289, TESTI2012308, TESTI2012356, TESTI2012387, TESTI2012394, TESTI2012444, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257,

TESTI2013264, TESTI2013268, TESTI2013295, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988,

TESTI2015042, TESTI2015092, TESTI2015105, TESTI2015180, TESTI2015246, TESTI2015249, TESTI2015327, TESTI2015331, TESTI2015335, TESTI2015350, TESTI2015375, TESTI2015437, TESTI2015590, TESTI2015610, TESTI2015626, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896,

TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018014, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018462, TESTI2018475, TESTI2018482, TESTI2018521, TESTI2018565, TESTI2018581,

TESTI2018611, TESTI2018687, TESTI2018697, TESTI2018838, TESTI2018867, TESTI2018941, TESTI2018957, TESTI2018974, TESTI2018975, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426,

TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021124, TESTI2021138, TESTI2021315, TESTI2021358, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323,

TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498,

TESTI2024560, TESTI2024567, TESTI2024586, TESTI2024598, TESTI2024641, TESTI2024648, TESTI2024718, TESTI2024744, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025448, TESTI2025454, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196,

TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562, TESTI2027615, TESTI2027645, TESTI2027730, TESTI2027736,

TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754,

TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033441, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718,

TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081,

TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707,

TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958,

TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046456, TESTI2046535, TESTI2046536, TESTI2046552, TESTI2046569,

TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767,

TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600,

TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002072, TESTI4002141, TESTI4002195, TESTI4002202, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003150, TESTI4003179, TESTI4003279, TESTI4003319,

TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441,

TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034,

TESTI4009123, TESTI4009160, TESTI4009215, TESTI4009283, TESTI4009286, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258,

TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013474, TESTI4013602, TESTI4013667, TESTI4013675, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977,

TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555,

TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344,

TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904,

TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498,

TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037156, TESTI4037188, TESTI4037228, TESTI4037244, TESTI4037727, TESTI4037949, TESTI4038047, TESTI4038099, TESTI4038156, TESTI4038223, TESTI4038258, TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800,

TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043140, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168,

TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000016, THYMU1000017, THYMU1000025, THYMU1000029, THYMU1000032, THYMU1000033, THYMU1000041, THYMU1000060, THYMU1000074, THYMU1000083, THYMU1000098, THYMU1000103, THYMU1000105, THYMU1000136, THYMU1000142, THYMU1000144,

THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000331, THYMU1000336, THYMU1000359, THYMU1000366, THYMU1000374, THYMU1000382, THYMU1000393, THYMU1000394, THYMU1000399, THYMU1000426, THYMU1000428, THYMU1000440, THYMU1000459, THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231,

THYMU2000236, THYMU2000276, THYMU2000280, THYMU2000300, THYMU2000317, THYMU2000368, THYMU2000369, THYMU2000418, THYMU2000440, THYMU2000489, THYMU2000492, THYMU2000519, THYMU2000570, THYMU2000602, THYMU2000660, THYMU2000671, THYMU2000672, THYMU2000684, THYMU2000702, THYMU2000767, THYMU2000775, THYMU2000800, THYMU2000879, THYMU2000932, THYMU2000946, THYMU2000950, THYMU2000971, THYMU2000982, THYMU2000990, THYMU2001007, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556,

THYMU2001622, THYMU2001727, THYMU2001819, THYMU2001825, THYMU2001839, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002583, THYMU2002695, THYMU2002745, THYMU2002789, THYMU2002792, THYMU2002815, THYMU2002841, THYMU2002852, THYMU2002910, THYMU2002950, THYMU2002983, THYMU2003012, THYMU2003069, THYMU2003107, THYMU2003124, THYMU2003166, THYMU2003232, THYMU2003242, THYMU2003249, THYMU2003270,

THYMU2003282, THYMU2003287, THYMU2003336, THYMU2003366, THYMU2003397, THYMU2003419, THYMU2003440, THYMU2003446, THYMU2003470, THYMU2003479, THYMU2003499, THYMU2003542, THYMU2003632, THYMU2003650, THYMU2003700, THYMU2003760, THYMU2003803, THYMU2003855, THYMU2003891, THYMU2003907, THYMU2003932, THYMU2003981, THYMU2004003, THYMU2004008, THYMU2004042, THYMU2004108, THYMU2004111, THYMU2004123, THYMU2004139, THYMU2004152, THYMU2004169, THYMU2004320, THYMU2004336, THYMU2004344, THYMU2004356, THYMU2004410, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004916,

THYMU2004986, THYMU2005001, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005507, THYMU2005545, THYMU2005546, THYMU2005574, THYMU2005576, THYMU2005748, THYMU2005759, THYMU2005807, THYMU2005815, THYMU2005855, THYMU2005881, THYMU2005948, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006505, THYMU2006610, THYMU2006666, THYMU2006712, THYMU2006813,

THYMU2006913, THYMU2006946, THYMU2006965, THYMU2007025, THYMU2007036, THYMU2007060, THYMU2007065, THYMU2007095, THYMU2007112, THYMU2007146, THYMU2007179, THYMU2007258, THYMU2007307, THYMU2007308, THYMU2007339, THYMU2007350, THYMU2007384, THYMU2007415, THYMU2007467, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007886, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282,

THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009658, THYMU2009792, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010448, THYMU2010519, THYMU2010637,

THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012024, THYMU2012073, THYMU2012104, THYMU2012113, THYMU2012273, THYMU2012401, THYMU2012492, THYMU2012507, THYMU2012565, THYMU2012574, THYMU2012631,

THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013136, THYMU2013170, THYMU2013364, THYMU2013386, THYMU2013426, THYMU2013578, THYMU2013648, THYMU2013705, THYMU2013757, THYMU2013779, THYMU2013863, THYMU2013916, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441,

THYMU2015479, THYMU2015762, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018126, THYMU2018189, THYMU2018197, THYMU2018384, THYMU2018455, THYMU2018547, THYMU2018565, THYMU2018639, THYMU2018673, THYMU2018721,

THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042,

THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796,

THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032014, THYMU2032035, THYMU2032080, THYMU2032150, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101,

THYMU2035319, THYMU2035321, THYMU2035388, THYMU2035400, THYMU2035554, THYMU2035710, THYMU2035735, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029,

THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763,

THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081,

THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013386, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015759, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856,

THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706,

THYMU3030710, THYMU3030752, THYMU3031146, THYMU3031402, THYMU3031610, THYMU3031612, THYMU3031868, THYMU3031878, THYMU3032032, THYMU3032798, THYMU3032867, THYMU3033402, THYMU3033630, THYMU3033649, THYMU3033754, THYMU3033759, THYMU3034078, THYMU3034099, THYMU3034453, THYMU3034616, THYMU3034671, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970,

THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542,

THYMU3047760, THYMU3047891, TKIDN1000001, TKIDN1.000044, TKIDN1000062, TKIDN1000066, TKIDN1000164, TKIDN1000171, TKIDN1000192, TKIDN2000127, TKIDN2000240, TKIDN2000246, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2002738, TKIDN2003044, TKIDN2003059, TKIDN2003062, TKIDN2003078, TKIDN2003366, TKIDN2003396, TKIDN2003413, TKIDN2003597, TKIDN2003601, TKIDN2004386, TKIDN2004458, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641,

TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015423, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002046, TLIVE2002336, TLIVE2002338, TLIVE2002562, TLIVE2002690, TLIVE2002882, TLIVE2003197, TLIVE2003200,

TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800,

TRACH1000011, TRACH1000018, TRACH1000030, TRACH1000038, TRACH1000057, TRACH1000063, TRACH1000074, TRACH1000100, TRACH1000106, TRACH1000125, TRACH1000140, TRACH1000144, TRACH1000181, TRACH1000193, TRACH1000205, TRACH1000212, TRACH1000240, TRACH2000002, TRACH2000015, TRACH2000024, TRACH2000032, TRACH2000045, TRACH2000081, TRACH2000083, TRACH2000148, TRACH2000185, TRACH2000196, TRACH2000235, TRACH2000237, TRACH2000243, TRACH2000250, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000420, TRACH2000461, TRACH2000472, TRACH2000496, TRACH2000497, TRACH2000502, TRACH2000522, TRACH2000559,

TRACH2000660, TRACH2000665, TRACH2000675, TRACH2000677, TRACH2000702, TRACH2000703, TRACH2000767, TRACH2000775, TRACH2000780, TRACH2000807, TRACH2000862, TRACH2000873, TRACH2000894, TRACH2000898, TRACH2000920, TRACH2000923, TRACH2000926, TRACH2000944, TRACH2000959, TRACH2000972, TRACH2000981, TRACH2000986, TRACH2001037, TRACH2001060, TRACH2001085, TRACH2001101, TRACH2001109, TRACH2001121, TRACH2001147, TRACH2001154, TRACH2001188, TRACH2001192, TRACH2001249, TRACH2001275, TRACH2001314, TRACH2001335, TRACH2001339, TRACH2001341, TRACH2001347, TRACH2001395, TRACH2001400, TRACH2001403, TRACH2001416, TRACH2001428, TRACH2001432, TRACH2001443, TRACH2001463, TRACH2001474, TRACH2001478, TRACH2001523,

TRACH2001525, TRACH2001532, TRACH2001549, TRACH2001592, TRACH2001596, TRACH2001612, TRACH2001621, TRACH2001646, TRACH2001651, TRACH2001684, TRACH2001810, TRACH2001898, TRACH2001933, TRACH2001968, TRACH2001996, TRACH2002043, TRACH2002054, TRACH2002092, TRACH2002100, TRACH2002138, TRACH2002333, TRACH2002537, TRACH2002664, TRACH2002784, TRACH2002803, TRACH2002840, TRACH2002847, TRACH2002853, TRACH2002862, TRACH2002954, TRACH2002960, TRACH2002988, TRACH2003070, TRACH2003272, TRACH2003323, TRACH2003347, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292,

TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005720, TRACH2005769, TRACH2005796, TRACH2005800, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007754, TRACH2007816, TRACH2007894,

TRACH2007969, TRACH2007988, TRACH2008081, TRACH2008112, TRACH2008113, TRACH2008127, TRACH2008130, TRACH2008278, TRACH2008300, TRACH2008472, TRACH2008478, TRACH2008540, TRACH2008583, TRACH2008597, TRACH2008716, TRACH2008820, TRACH2008891, TRACH200892.1, TRACH2009006, TRACH2009060, TRACH20091.07, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771,

TRACH2010824, TRACH2010883, TRACH2010965, TRACH2010974, TRACH2011057, TRACH2011093, TRACH2011290, TRACH2011293, TRACH2011302, TRACH2011500, TRACH2011574, TRACH2011894, TRACH2011956, TRACH2012000, TRACH2012138, TRACH2012242, TRACH2012298, TRACH2012370, TRACH2012376, TRACH2012387, TRACH2012497, TRACH2012536, TRACH2012562, TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014077, TRACH2014082, TRACH2014268, TRACH2014284, TRACH2014337, TRACH2014356,

TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015823, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980,

TRACH2018034, TRACH2018084, TRACH2018262, TRACH2018273, TRACH2018278, TRACH2018317, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299,

TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025535, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892,

TRACH3000926, TRACH3001119, TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003547, TRACH3003586, TRACH3003683, TRACH3003780, TRACH3003805, TRACH3003832, TRACH3003872, TRACH3004068, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840,

TRACH3004931, TRACH3004934, TRACH3005072, TRACH3005085, TRACH3005102, TRACH3005173, TRACH3005191, TRACH3005211, TRACH3005274, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006800, TRACH3006889, TRACH3006985, TRACH3007097, TRACH3007268, TRACH3007274, TRACH3007277, TRACH3007391, TRACH3007541, TRACH3007595, TRACH3007625, TRACH3007627, TRACH3007689, TRACH3007766, TRACH3007866, TRACH3007995, TRACH3008042, TRACH3008061, TRACH3008093,

TRACH3008201, TRACH3008508, TRACH3008535, TRACH3008629, TRACH3008632, TRACH3008688, TRACH3008697, TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043,

TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058,

TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428,

TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400,

TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063,

TRACH3037067, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143,

UMVEN1000156, UMVEN2000046, UMVEN2000069, UMVEN2000121, UMVEN2000152, UMVEN2000354, UMVEN2000453, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000183, UTERU1000187, UTERU1000192, UTERU1000249, UTERU1000337, UTERU1000339, UTERU1000384, UTERU2000023, UTERU2000047, UTERU2000074, UTERU2000099, UTERU2000154, UTERU2000197, UTERU2000218, UTERU2000238, UTERU2000243, UTERU2000260, UTERU2000300, -UTERU2000329, UTERU2000332, UTERU2000338, UTERU2000349, UTERU2000377, UTERU2000393, UTERU2000418, UTERU2000465, UTERU2000517,

UTERU2000524, UTERU2000537, UTERU2000539, UTERU2000541, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002198, UTERU2002332, UTERU2002410, UTERU2002473, UTERU2002547, UTERU2002662, UTERU2002693, UTERU2002733, UTERU2002736, UTERU2002737, UTERU2002826, UTERU2002841, UTERU2002964, UTERU2002993, UTERU2003035, UTERU2003057, UTERU2003126, UTERU2003135,

UTERU2003321, UTERU2003399, UTERU2003411, UTERU2003456, UTERU2003577, UTERU2003704, UTERU2003926, UTERU2003973, UTERU2004015, UTERU2004039, UTERU2004061, UTERU2004073, UTERU2004163, UTERU2004197, UTERU2004299, UTERU2004461, UTERU2004520, UTERU2004564, UTERU2004664, UTERU2004688, UTERU2004698, UTERU2004807, UTERU2004861, UTERU2004929, UTERU2005004, UTERU2005050, UTERU2005069, UTERU2005074, UTERU2005179, UTERU2005346, UTERU2005354, UTERU2005446, UTERU2005449, UTERU2005450, UTERU2005533, UTERU2005593, UTERU2005601, UTERU2005621, UTERU2005664, UTERU2005822, UTERU2005903, UTERU2005905, UTERU2006103, UTERU2006115, UTERU2006137, UTERU2006182, UTERU2006412, UTERU2006429, UTERU2006486, UTERU2006524,

UTERU2006547, UTERU2006568, UTERU2006593, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639, UTERU2007724, UTERU2007942, UTERU2008018, UTERU2008019, UTERU2008027, UTERU2008040, UTERU2008077, UTERU2008130, UTERU2008302, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008939, UTERU2008962, UTERU2009094, UTERU2009120, UTERU2009131, UTERU2009147, UTERU2009206, UTERU2009283, UTERU2009414, UTERU2009435, UTERU2009483, UTERU2009510, UTERU2009538,

UTERU2009540, UTERU2009776, UTERU2009904, UTERU2009951, UTERU2009972, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012252, UTERU2012286, UTERU2012333, UTERU2012407, UTERU2012526, UTERU2012581, UTERU2012610, UTERU2012615, UTERU2012688, UTERU2012703, UTERU2012715, UTERU2012741, UTERU2012767, UTERU2012786,

UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001, UTERU2014024, UTERU2014167, UTERU2014223, UTERU2014398, UTERU2014464, UTERU2014548, UTERU2014601, UTERU2014631, UTERU2014668, UTERU2014678, UTERU2014728, UTERU2014998, UTERU2015062, UTERU2015087, UTERU2015108, UTERU2015190, UTERU2015198, UTERU2015202, UTERU2015405, UTERU2015640, UTERU2015830, UTERU2015880, UTERU2016147, UTERU2016157, UTERU2016426, UTERU2016464, UTERU2016669, UTERU2016757, UTERU2016761, UTERU2016799, UTERU2016822, UTERU2016896, UTERU2016902,

UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019005, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292,

UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742,

UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423,

UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660,

UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690,

UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172,

UTERU3018255, UTERU3018616, UTERU3018711, UTERU3019078

Of the 17,176 genes including the cDNAs of the present invention, genes, whose expression levels were high enough to detect in each library of HELAC, HHDPC, HLUNG, HSYRA, IMR32, JCMLC, and KIDNE, were analyzed for the expression frequencies. The analysis results are described below. Each is a relative value, where the expression level over all tissues is taken as 100. The larger this value, the greater the level of expression.

TABLE 32

| Clone Name | HELAC | HHDPC | HLUNG | HSYRA | IMR32 | JCMLC | KIDNE |
|---|---|---|---|---|---|---|---|
| 3NB691000113 | 0 | 0 | 0 | 50.609 | 0 | 0 | 0 |
| 3NB691000116 | 0 | 11.451 | 0 | 0 | 0 | 0 | 0 |
| 3NB691000173 | 0 | 0 | 0 | 0 | 11.145 | 0 | 0 |
| 3NB692000281 | 0 | 0 | 0 | 0 | 3.107 | 0 | 0 |
| 3NB692000330 | 0 | 0 | 1.618 | 0 | 3.113 | 0 | 1.538 |
| 3NB692000374 | 0 | 7.636 | 3.978 | 0 | 0 | 0 | 0 |
| 3NB692000545 | 0 | 0 | 0 | 0 | 9.955 | 0 | 0 |
| 3NB692000912 | 0 | 0 | 0 | 0 | 3.316 | 0 | 1.638 |
| 3NB692001002 | 0 | 0 | 7.176 | 0 | 0 | 0 | 0 |
| 3NB692001022 | 0 | 0 | 0 | 0 | 4.390 | 0 | 0 |
| 3NB692001288 | 0 | 10.549 | 0 | 0 | 0 | 0 | 5.223 |
| 3NB692001334 | 0 | 0 | 0.985 | 2.006 | 7.577 | 0 | 0 |
| 3NB692001442 | 0 | 0 | 0 | 9.028 | 4.262 | 0 | 0 |
| 3NB692001459 | 0 | 0 | 0 | 0 | 19.948 | 0 | 0 |
| 3NB692001511 | 0 | 0 | 31.576 | 0 | 0 | 0 | 0 |
| 3NB692001637 | 0 | 0 | 0 | 33.877 | 0 | 0 | 0 |
| ADRGL2002477 | 0 | 0 | 6.240 | 0 | 0 | 0 | 5.931 |
| ADRGL2005838 | 0 | 0 | 38.763 | 0 | 0 | 0 | 0 |
| ADRGL2012038 | 0 | 0 | 0 | 15.365 | 0 | 0 | 0 |
| ASTRO1000165 | 0 | 0 | 0 | 16.566 | 0 | 0 | 7.727 |
| ASTRO2000014 | 36.043 | 0 | 0 | 0 | 0 | 0 | 0 |
| ASTRO2000046 | 0 | 2.873 | 0 | 3.050 | 0 | 0 | 0 |
| ASTRO2000480 | 0 | 0 | 0 | 0 | 9.990 | 0 | 0 |
| ASTRO2000801 | 0 | 0 | 0 | 0 | 3.600 | 0 | 0 |
| ASTRO2000914 | 0 | 0 | 7.139 | 0 | 0 | 0 | 6.786 |
| ASTRO2001029 | 0 | 0 | 9.198 | 0 | 0 | 0 | 0 |
| ASTRO2001076 | 0 | 0 | 0 | 0 | 0 | 39.073 | 0 |
| ASTRO2001806 | 0 | 10.074 | 0 | 0 | 0 | 0 | 0 |
| ASTRO2001823 | 0 | 10.221 | 0 | 0 | 0 | 0 | 0 |
| ASTRO2002459 | 0 | 0 | 0 | 6.543 | 0 | 0 | 0 |
| ASTRO2003024 | 0 | 0 | 3.476 | 0 | 0 | 0 | 0 |
| ASTRO2003632 | 0 | 2.925 | 12.190 | 0 | 0 | 0 | 0 |
| ASTRO2005687 | 0 | 0 | 0 | 24.993 | 0 | 0 | 0 |
| ASTRO2007047 | 0 | 0 | 15.330 | 0 | 0 | 0 | 0 |
| ASTRO2010819 | 0 | 0 | 0 | 0 | 0 | 0 | 2.346 |
| ASTRO2012552 | 0 | 0 | 0 | 0 | 10.431 | 0 | 0 |
| ASTRO2013585 | 0 | 0 | 0 | 3.644 | 0 | 0 | 1.700 |
| ASTRO2016491 | 0 | 0 | 1.847 | 0 | 0 | 0 | 0 |
| ASTRO2018169 | 82.898 | 0 | 0 | 0 | 0 | 0 | 0 |
| BGGI11000123 | 61.509 | 0 | 0 | 0 | 0 | 0 | 0 |
| BGGI12000533 | 0 | 15.896 | 0 | 0 | 0 | 0 | 0 |
| BGGI12000616 | 0 | 0 | 0 | 0 | 0 | 0 | 3.117 |
| BGGI12000839 | 0 | 0 | 2.705 | 0 | 0 | 0 | 0 |
| BGGI12001097 | 0 | 0 | 4.122 | 0 | 0 | 0 | 3.918 |
| BGGI12001682 | 0 | 0 | 0 | 0 | 3.644 | 0 | 0 |
| BLADE2002744 | 0 | 0 | 0 | 0 | 0 | 67.097 | 0 |
| BLADE2006830 | 0 | 0 | 0 | 0 | 0 | 46.211 | 0 |
| BLADE2007589 | 0 | 0 | 18.131 | 0 | 0 | 0 | 0 |
| BNGH41000153 | 0 | 0 | 17.235 | 0 | 0 | 0 | 0 |
| BNGH41000198 | 0 | 0 | 7.620 | 0 | 7.331 | 0 | 0 |
| BNGH42000130 | 91.923 | 0 | 0 | 0 | 0 | 0 | 0 |
| BNGH42000360 | 0 | 0 | 0 | 0 | 1.110 | 0 | 0 |
| BNGH42000739 | 0 | 0 | 2.282 | 0 | 0 | 0 | 0 |
| BNGH42007709 | 0 | 0 | 3.377 | 0 | 0.812 | 0 | 0.802 |
| BNGH42007788 | 0 | 0 | 0 | 23.009 | 0 | 0 | 0 |
| BRACE1000131 | 0 | 7.334 | 0 | 7.785 | 0 | 0 | 0 |
| BRACE1000159 | 0 | 0 | 0 | 0 | 6.515 | 0 | 0 |
| BRACE1000186 | 0 | 15.224 | 0 | 0 | 0 | 0 | 0 |
| BRACE2000245 | 0 | 0 | 0 | 0 | 0 | 0 | 30.833 |
| BRACE2000505 | 0 | 0 | 0 | 91.327 | 0 | 0 | 0 |
| BRACE2000525 | 0 | 0 | 0 | 0 | 0 | 0 | 5.664 |
| BRACE2000969 | 0 | 0 | 83.786 | 0 | 0 | 0 | 0 |
| BRACE2001117 | 0 | 0 | 0 | 0 | 19.410 | 0 | 9.589 |
| BRACE2001497 | 0 | 0 | 0 | 13.927 | 0 | 0 | 0 |
| BRACE2001692 | 0 | 0 | 0 | 0 | 2.137 | 0 | 0 |
| BRACE2001855 | 0 | 0 | 12.661 | 8.600 | 0 | 0 | 0 |
| BRACE2001892 | 0 | 0 | 0 | 0 | 1.069 | 0 | 0 |
| BRACE2001971 | 0 | 0 | 0 | 0 | 7.914 | 0 | 0 |
| BRACE2002081 | 0 | 0 | 70.380 | 0 | 0 | 0 | 0 |
| BRACE2002091 | 0 | 0 | 0 | 0 | 7.451 | 0 | 7.362 |
| BRACE2002392 | 0 | 0 | 0 | 0 | 6.082 | 0 | 0 |
| BRACE2002441 | 0 | 7.930 | 0 | 0 | 0 | 30.961 | 0 |
| BRACE2002685 | 0 | 0 | 0 | 0 | 0 | 0 | 11.950 |
| BRACE2002860 | 0 | 0 | 0 | 0 | 0 | 0 | 8.777 |
| BRACE2003172 | 0 | 0 | 0 | 0 | 0 | 0 | 4.532 |
| BRACE2003217 | 0 | 0 | 0 | 0 | 15.210 | 0 | 0 |
| BRACE2003254 | 0 | 0 | 3.321 | 0 | 3.195 | 0 | 0 |
| BRACE2003527 | 0 | 18.150 | 0 | 0 | 0 | 0 | 0 |
| BRACE2003827 | 0 | 0 | 0 | 0 | 3.091 | 0 | 0 |
| BRACE2003846 | 57.200 | 0 | 0 | 0 | 0 | 0 | 0 |
| BRACE2003892 | 0 | 0 | 8.005 | 0 | 0 | 0 | 0 |
| BRACE2003904 | 0 | 0 | 0 | 0 | 0 | 0 | 9.038 |
| BRACE2005099 | 0 | 0 | 0 | 0 | 0 | 0 | 9.269 |
| BRACE2005231 | 0 | 0 | 0 | 0 | 0 | 0 | 11.130 |
| BRACE2005434 | 0 | 0 | 0 | 0 | 2.433 | 0 | 0 |
| BRACE2005448 | 0 | 0 | 0 | 0 | 0 | 0 | 23.706 |
| BRACE2005681 | 0 | 0 | 0 | 0 | 3.522 | 0 | 0 |
| BRACE2006055 | 0 | 28.171 | 0 | 0 | 0 | 0 | 6.974 |
| BRACE2006089 | 0 | 28.709 | 0 | 0 | 0 | 0 | 0 |
| BRACE2006258 | 0 | 0 | 3.004 | 0 | 8.671 | 0 | 2.856 |
| BRACE2006397 | 0 | 0 | 6.496 | 0 | 0 | 8.114 | 1.029 |
| BRACE2006514 | 0 | 0 | 0 | 6.493 | 0 | 0 | 3.029 |
| BRACE2006871 | 0 | 0 | 0 | 0 | 3.528 | 0 | 0 |
| BRACE2006900 | 0 | 0 | 0 | 0 | 0 | 0 | 1.794 |
| BRACE2006911 | 0 | 0 | 68.109 | 0 | 0 | 0 | 0 |
| BRACE2007089 | 74.315 | 0 | 0 | 0 | 0 | 0 | 0 |
| BRACE2007401 | 0 | 0 | 0 | 0 | 6.449 | 0 | 0 |
| BRACE2007518 | 0 | 0 | 8.324 | 0 | 0 | 0 | 0 |
| BRACE2007577 | 0 | 0 | 3.591 | 0 | 0 | 0 | 0 |
| BRACE2008097 | 0 | 0 | 0 | 0 | 0 | 0 | 16.738 |
| BRACE2008251 | 0 | 0 | 49.689 | 0 | 0 | 0 | 0 |
| BRACE2008380 | 0 | 0 | 0 | 0 | 6.894 | 0 | 0 |
| BRACE2008385 | 0 | 0 | 7.660 | 0 | 0 | 0 | 0 |
| BRACE2008495 | 0 | 0 | 83.786 | 0 | 0 | 0 | 0 |
| BRACE2008706 | 0 | 0 | 0 | 49.117 | 0 | 0 | 0 |
| BRACE2008968 | 0 | 0 | 2.133 | 4.346 | 2.052 | 0 | 2.027 |
| BRACE2009037 | 0 | 2.056 | 2.142 | 2.182 | 0 | 0 | 1.018 |
| BRACE2009620 | 0 | 0 | 70.380 | 0 | 0 | 0 | 0 |
| BRACE2011265 | 0 | 0 | 0 | 0 | 13.090 | 0 | 0 |
| BRACE2012061 | 0 | 0 | 0 | 0 | 0 | 0 | 58.734 |
| BRACE2018337 | 0 | 16.771 | 0 | 0 | 0 | 0 | 0 |
| BRACE2021478 | 0 | 0 | 0 | 0 | 0 | 0 | 45.131 |
| BRACE2039624 | 0 | 0 | 3.548 | 0 | 0 | 0 | 3.373 |
| BRACE3004150 | 0 | 27.348 | 0 | 0 | 0 | 0 | 0 |
| BRACE3004371 | 0 | 0 | 0 | 0 | 0 | 0 | 13.097 |
| BRACE3005938 | 0 | 7.754 | 4.039 | 0 | 0 | 0 | 0 |
| BRACE3010428 | 0 | 0 | 0 | 17.112 | 0 | 0 | 3.991 |
| BRALZ2009482 | 0 | 0 | 1.363 | 0 | 0 | 0 | 5.182 |
| BRAMY2000151 | 0 | 0 | 0 | 0 | 77.787 | 0 | 0 |
| BRAMY2000354 | 0 | 0 | 0 | 0 | 0 | 0 | 4.791 |
| BRAMY2000388 | 0 | 0 | 0 | 0 | 0 | 0 | 21.330 |
| BRAMY2009934 | 0 | 0 | 0 | 0 | 20.859 | 0 | 0 |
| BRAMY2010208 | 0 | 0 | 0 | 0 | 0 | 0 | 63.370 |
| BRAMY2015911 | 0 | 0 | 0 | 0 | 0 | 0 | 51.943 |
| BRAMY2016325 | 0 | 26.531 | 0 | 0 | 0 | 0 | 0 |
| BRAMY2019554 | 0 | 0 | 0 | 0 | 0 | 21.938 | 0 |
| BRAMY2020412 | 0 | 0 | 0 | 80.233 | 0 | 0 | 0 |
| BRAMY2020427 | 0 | 0 | 2.172 | 0 | 4.179 | 0 | 2.064 |
| BRAMY2021310 | 0 | 87.482 | 0 | 0 | 0 | 0 | 0 |
| BRAMY2022786 | 0 | 17.573 | 0 | 0 | 0 | 0 | 0 |
| BRAMY2024004 | 0 | 0 | 0 | 0 | 0 | 76.038 | 0 |
| BRAMY2025230 | 0 | 0 | 0 | 73.283 | 0 | 0 | 0 |
| BRAMY2026778 | 0 | 34.114 | 0 | 0 | 0 | 0 | 0 |
| BRAMY2028914 | 0 | 0 | 0 | 0 | 62.506 | 0 | 0 |
| BRAMY2031516 | 0 | 0 | 27.091 | 0 | 0 | 0 | 0 |
| BRAMY2038832 | 0 | 0 | 44.706 | 0 | 0 | 0 | 0 |
| BRAMY2039630 | 0 | 0 | 0 | 0 | 0 | 0 | 60.511 |
| BRAMY2040095 | 0 | 0 | 1.254 | 0 | 0 | 18.794 | 0 |
| BRAMY2047420 | 0 | 0 | 0 | 0 | 7.149 | 0 | 0 |
| BRAMY3004364 | 0 | 0 | 0 | 0 | 0 | 0 | 23.683 |
| BRAMY3004377 | 0 | 5.840 | 3.042 | 6.199 | 2.926 | 0 | 0 |
| BRAMY3008044 | 0 | 0 | 0 | 0 | 0 | 0 | 6.254 |
| BRAMY3010008 | 0 | 0 | 2.135 | 0 | 0 | 0 | 0 |
| BRAMY3010492 | 0 | 0 | 21.124 | 0 | 0 | 0 | 0 |
| BRAWH1000040 | 0 | 0 | 0 | 5.143 | 0 | 0 | 0 |
| BRAWH1000083 | 0 | 0 | 0 | 0 | 0 | 0 | 15.796 |
| BRAWH1000093 | 0 | 3.248 | 0 | 0 | 0 | 12.679 | 0 |
| BRAWH2000417 | 0 | 0 | 2.266 | 0 | 0 | 0 | 0 |
| BRAWH2000460 | 0 | 4.297 | 2.238 | 0 | 0 | 0 | 2.128 |
| BRAWH2000522 | 0 | 0 | 0 | 0 | 12.791 | 0 | 0 |
| BRAWH2000588 | 0 | 0 | 0 | 9.377 | 0 | 0 | 0 |
| BRAWH2000633 | 0 | 0 | 0 | 0 | 4.678 | 0 | 0 |
| BRAWH2000636 | 0 | 0 | 0 | 3.950 | 0 | 0 | 0 |
| BRAWH2000697 | 0 | 0 | 3.710 | 0 | 0 | 0 | 3.527 |
| BRAWH2000944 | 0 | 0 | 19.134 | 0 | 0 | 0 | 0 |
| BRAWH2001092 | 0 | 3.713 | 1.289 | 1.314 | 0.620 | 0 | 1.225 |
| BRAWH2001166 | 0 | 0 | 11.578 | 0 | 0 | 0 | 0 |
| BRAWH2001239 | 0 | 51.373 | 0 | 0 | 0 | 0 | 0 |
| BRAWH2001424 | 0 | 0 | 0 | 0 | 0 | 0 | 17.750 |
| BRAWH2001461 | 0 | 0 | 13.038 | 0 | 0 | 0 | 0 |
| BRAWH2003025 | 0 | 0 | 11.121 | 0 | 0 | 0 | 0 |
| BRAWH2003693 | 0 | 24.090 | 0 | 0 | 0 | 0 | 0 |
| BRAWH2003818 | 0 | 0 | 0 | 22.621 | 0 | 0 | 0 |
| BRAWH2004078 | 0 | 0 | 0 | 0 | 0 | 0 | 18.371 |
| BRAWH2005068 | 0 | 0 | 0 | 0 | 2.839 | 0 | 0 |
| BRAWH2006996 | 0 | 0 | 0 | 25.423 | 0 | 0 | 0 |
| BRAWH2008255 | 0 | 0 | 0 | 63.154 | 0 | 0 | 0 |
| BRAWH2009307 | 0 | 0 | 7.303 | 0 | 0 | 0 | 0 |
| BRAWH2011812 | 0 | 0 | 0 | 0 | 47.789 | 0 | 0 |
| BRAWH2012538 | 0 | 0 | 0 | 0 | 0 | 0 | 35.370 |
| BRAWH2014188 | 0 | 0 | 2.626 | 5.351 | 0 | 0 | 0 |
| BRAWH2015349 | 0 | 0 | 0 | 0 | 0 | 0 | 38.345 |
| BRAWH2016269 | 0 | 0 | 0 | 0 | 4.652 | 0 | 4.596 |
| BRAWH2018526 | 0 | 0 | 0 | 0 | 1.086 | 0 | 0 |
| BRAWH2018729 | 0 | 0 | 0 | 22.271 | 0 | 0 | 0 |
| BRAWH3002853 | 0 | 10.416 | 2.713 | 0 | 0 | 0 | 0 |
| BRAWH3023172 | 80.380 | 0 | 0 | 0 | 3.232 | 0 | 0 |
| BRCAN2000639 | 0 | 0 | 61.519 | 0 | 0 | 0 | 0 |
| BRCAN2002173 | 0 | 0 | 0 | 0 | 0 | 0 | 6.932 |
| BRCAN2003944 | 0 | 1.983 | 0 | 4.210 | 0 | 0 | 0.982 |
| BRCAN2021028 | 0 | 0 | 38.589 | 0 | 0 | 0 | 0 |
| BRCAN2024572 | 0 | 0 | 0 | 20.413 | 0 | 0 | 0 |
| BRCAN2026197 | 0 | 10.993 | 0 | 0 | 0 | 0 | 0 |
| BRCAN2028036 | 0 | 0 | 8.855 | 0 | 0 | 0 | 0 |
| BRCOC2010123 | 0 | 0 | 0 | 8.188 | 0 | 0 | 0 |
| BRCOC2011506 | 83.518 | 0 | 0 | 0 | 0 | 0 | 0 |
| BRCOC2014013 | 0 | 0 | 0 | 0 | 0 | 0 | 18.907 |
| BRHIP1000104 | 0 | 0 | 2.397 | 0 | 0 | 0 | 0 |
| BRHIP2000534 | 0 | 0 | 0 | 2.558 | 0 | 18.818 | 0 |
| BRHIP2000553 | 0 | 18.094 | 0 | 0 | 0 | 0 | 0 |
| BRHIP2000819 | 0 | 0 | 7.923 | 0 | 0 | 0 | 0 |
| BRHIP2001099 | 0 | 0 | 25.425 | 0 | 0 | 0 | 24.167 |
| BRHIP2007305 | 0 | 0 | 4.152 | 8.461 | 0 | 0 | 3.947 |
| BRHIP2011576 | 0 | 0 | 0 | 0 | 0 | 0 | 66.835 |
| BRHIP2012627 | 0 | 0 | 0 | 0 | 55.428 | 0 | 0 |
| BRHIP2020509 | 0 | 0 | 0 | 0 | 0 | 0 | 65.282 |
| BRHIP2021870 | 0 | 0 | 0 | 0 | 0 | 0 | 15.479 |
| BRHIP2023272 | 0 | 0 | 12.379 | 0 | 0 | 0 | 0 |
| BRHIP2023888 | 0 | 0 | 1.350 | 0 | 0 | 10.115 | 1.283 |
| BRHIP2024398 | 0 | 0 | 30.880 | 0 | 0 | 0 | 0 |
| BRHIP3000111 | 0 | 0 | 0 | 7.652 | 0 | 0 | 0 |
| BRHIP3006717 | 0 | 11.985 | 0 | 0 | 0 | 0 | 0 |
| BRSSN2003012 | 0 | 0 | 15.866 | 32.333 | 0 | 0 | 0 |
| BRSSN2007464 | 0 | 0 | 0 | 0 | 14.440 | 0 | 0 |
| BRSSN2009518 | 0 | 0 | 0 | 25.748 | 0 | 0 | 0 |
| BRSTN2011961 | 0 | 4.078 | 2.124 | 8.658 | 0 | 0 | 0 |
| BRSTN2012069 | 0 | 0.133 | 0.763 | 0.848 | 2.503 | 0 | 0.429 |
| BRSTN2012174 | 0 | 0 | 0 | 1.512 | 4.996 | 0 | 0 |
| BRSTN2013171 | 0 | 0 | 25.419 | 0 | 0 | 0 | 0 |
| BRSTN2016470 | 0 | 0 | 0.803 | 0 | 1.545 | 0 | 0.763 |
| BRSTN2016892 | 0 | 0 | 0 | 0 | 10.529 | 0 | 0 |
| BRSTN2017104 | 0 | 0 | 0 | 0 | 6.880 | 0 | 0 |
| BRTHA2005864 | 0 | 0 | 5.951 | 0 | 0 | 0 | 0 |
| BRTHA2012714 | 0 | 0 | 9.194 | 0 | 0 | 0 | 0 |
| BRTHA2016577 | 0 | 0 | 0 | 0 | 8.326 | 0 | 0 |
| BRTHA2027229 | 0 | 0 | 0 | 0 | 3.230 | 0 | 0 |
| BRTHA3010540 | 0 | 0 | 0 | 0 | 0 | 92.534 | 0 |
| CTONG1000052 | 0 | 0 | 2.216 | 0 | 0 | 0 | 0 |
| CTONG1000087 | 0 | 0 | 3.835 | 0 | 0 | 0 | 3.646 |
| CTONG1000094 | 0 | 0 | 1.428 | 0 | 1.374 | 0 | 0 |
| CTONG1000175 | 0 | 0 | 0 | 0 | 0 | 0 | 5.870 |
| CTONG2000469 | 0 | 20.562 | 0 | 0 | 0 | 0 | 0 |
| CTONG2001226 | 0 | 0 | 0 | 0 | 0 | 0 | 26.463 |
| CTONG2002453 | 0 | 0 | 0 | 0 | 27.393 | 0 | 0 |
| CTONG2002903 | 0 | 0 | 11.298 | 0 | 0 | 0 | 0 |
| CTONG2003298 | 0 | 5.110 | 0 | 0 | 0 | 0 | 0 |
| CTONG2003524 | 0 | 0 | 5.910 | 24.087 | 0 | 0 | 0 |
| CTONG2003937 | 0 | 0 | 0 | 0 | 0 | 0 | 65.178 |
| CTONG2004487 | 0 | 0 | 3.469 | 0 | 0 | 0 | 0 |
| CTONG2004550 | 0 | 0 | 0 | 0 | 0 | 0 | 65.178 |
| CTONG2005265 | 0 | 0 | 0 | 0 | 0 | 0 | 65.178 |
| CTONG2005553 | 0 | 0 | 0 | 0 | 2.527 | 19.687 | 0 |
| CTONG2006004 | 0 | 0 | 3.871 | 0 | 0 | 0 | 0 |
| CTONG2006562 | 0 | 0 | 0 | 0 | 9.880 | 0 | 0 |
| CTONG2006611 | 0 | 0 | 26.485 | 0 | 0 | 0 | 0 |
| CTONG2006932 | 0 | 7.748 | 0 | 0 | 0 | 0 | 0 |
| CTONG2007072 | 0 | 15.148 | 0 | 0 | 0 | 0 | 0 |
| CTONG2007078 | 0 | 0 | 0 | 0 | 4.509 | 0 | 0 |
| CTONG2007500 | 0 | 5.295 | 5.517 | 0 | 0 | 20.674 | 0 |
| CTONG2008269 | 0 | 47.906 | 0 | 0 | 0 | 0 | 0 |
| CTONG2008398 | 0 | 0 | 0 | 5.714 | 0 | 0 | 2.665 |
| CTONG2008506 | 0 | 0 | 0 | 0 | 0 | 0 | 6.561 |
| CTONG2010348 | 0 | 0 | 6.495 | 0 | 0 | 0 | 0 |
| CTONG2010821 | 0 | 0 | 0 | 0 | 0 | 0 | 2.491 |
| CTONG2012029 | 0 | 0 | 0 | 0 | 0 | 0 | 65.178 |
| CTONG2012158 | 0 | 0 | 0 | 0 | 32.190 | 0 | 0 |
| CTONG2012452 | 0 | 0 | 2.612 | 0 | 3.769 | 0 | 0 |
| CTONG2012607 | 0 | 0 | 0 | 0 | 0 | 47.544 | 0 |
| CTONG2012847 | 0 | 0 | 0 | 0 | 0 | 0 | 6.004 |
| CTONG2012996 | 0 | 20.559 | 10.709 | 0 | 0 | 0 | 0 |
| CTONG2013348 | 0 | 0 | 0 | 0 | 19.412 | 0 | 19.179 |
| CTONG2013907 | 0 | 0 | 0 | 0 | 7.607 | 0 | 7.516 |
| CTONG2015091 | 0 | 0 | 0 | 6.070 | 2.865 | 0 | 0 |
| CTONG2015518 | 0 | 0 | 48.707 | 0 | 0 | 0 | 0 |
| CTONG2016056 | 0 | 14.001 | 0 | 0 | 0 | 0 | 0 |
| CTONG2016575 | 0 | 3.501 | 1.824 | 0 | 0 | 0 | 0 |
| CTONG2016824 | 0 | 0 | 0 | 0 | 15.019 | 0 | 0 |
| CTONG2016869 | 0 | 0 | 9.687 | 0 | 0 | 0 | 0 |
| CTONG2016931 | 0 | 0 | 11.193 | 0 | 0 | 0 | 0 |
| CTONG2017429 | 0 | 0 | 0 | 0 | 25.465 | 0 | 0 |
| CTONG2017444 | 0 | 0 | 13.506 | 0 | 0 | 0 | 0 |
| CTONG2018334 | 0 | 0 | 26.829 | 0 | 0 | 0 | 0 |
| CTONG2020484 | 0 | 0 | 2.156 | 4.394 | 0 | 0 | 0 |
| CTONG2022835 | 0 | 12.080 | 0 | 0 | 0 | 0 | 0 |
| CTONG2028124 | 0 | 0 | 9.217 | 3.756 | 0 | 0 | 3.504 |
| CTONG3008894 | 0 | 0 | 0 | 0 | 0 | 0 | 2.534 |
| CTONG3009028 | 0 | 0 | 0 | 0 | 0 | 44.240 | 0 |
| CTONG3009239 | 0 | 0 | 0 | 10.791 | 0 | 0 | 0 |
| D3OST2002068 | 0 | 0 | 0.788 | 0 | 0 | 0 | 0 |
| D3OST2002648 | 0 | 0 | 0 | 0 | 0 | 82.485 | 0 |
| D6OST2000464 | 0 | 0 | 0 | 8.189 | 0 | 0 | 0 |
| D9OST2000031 | 0 | 0 | 0 | 0 | 0 | 0 | 16.470 |
| D9OST2002608 | 0 | 0 | 8.171 | 0 | 0 | 0 | 0 |
| D9OST2002673 | 0 | 0 | 0 | 0 | 0 | 80.305 | 0 |
| DFNES1000150 | 0 | 0 | 0 | 0 | 0 | 30.700 | 3.893 |
| DFNES2000011 | 0 | 0 | 0 | 4.087 | 0 | 0 | 0 |
| DFNES2000153 | 0 | 0 | 0 | 0 | 0 | 0 | 11.162 |
| DFNES2000268 | 0 | 0 | 8.100 | 0 | 3.896 | 0 | 0 |
| DFNES2002509 | 0 | 0 | 0 | 37.950 | 0 | 0 | 0 |
| DFNES2004371 | 0 | 0 | 0 | 11.792 | 0 | 0 | 0 |
| DFNES2005766 | 0 | 0 | 6.724 | 0 | 0 | 0 | 0 |
| DFNES2008088 | 0 | 18.511 | 0 | 0 | 0 | 0 | 9.165 |
| FCBBF1000023 | 0 | 49.531 | 0 | 0 | 0 | 0 | 0 |
| FCBBF1000027 | 0 | 2.579 | 0 | 2.738 | 1.293 | 10.070 | 1.277 |
| FCBBF1000121 | 0 | 0 | 36.439 | 0 | 0 | 0 | 0 |
| FCBBF1000182 | 0 | 0 | 0 | 0 | 0 | 0 | 59.128 |
| FCBBF1000294 | 0 | 0 | 3.949 | 0 | 0 | 0 | 0 |
| FCBBF1000466 | 0 | 0 | 2.336 | 0 | 0 | 0 | 0 |
| FCBBF1000506 | 0 | 0 | 0 | 0 | 65.635 | 0 | 0 |
| FCBBF1000563 | 0 | 0 | 0 | 0 | 0 | 0 | 5.236 |
| FCBBF1000760 | 0 | 0 | 0 | 0 | 0 | 18.002 | 2.283 |
| FCBBF2000199 | 0 | 0 | 0 | 0 | 9.872 | 0 | 0 |
| FCBBF2000885 | 0 | 0 | 5.630 | 0 | 5.416 | 0 | 0 |
| FCBBF2001088 | 0 | 0 | 0 | 0 | 0 | 0 | 56.526 |
| FCBBF2001105 | 0 | 0 | 0 | 0 | 0 | 0 | 21.165 |
| FCBBF2002349 | 0 | 0 | 0 | 0 | 0 | 0 | 13.763 |
| FCBBF2002801 | 0 | 0 | 0 | 0 | 9.158 | 0 | 0 |
| FCBBF2005966 | 0 | 0 | 34.736 | 0 | 0 | 0 | 0 |
| FCBBF3000110 | 0 | 0 | 23.687 | 0 | 0 | 0 | 0 |
| FCBBF3000115 | 0 | 0 | 14.638 | 0 | 0 | 0 | 0 |
| FCBBF3000434 | 0 | 0 | 6.790 | 0 | 6.533 | 0 | 0 |
| FCBBF3000536 | 0 | 0 | 0 | 0 | 0 | 73.279 | 0 |
| FCBBF3001470 | 0 | 0 | 4.979 | 0 | 4.790 | 0 | 0 |
| FCBBF3001632 | 0 | 10.870 | 0 | 0 | 5.447 | 0 | 0 |
| FCBBF3003435 | 0 | 0 | 0 | 66.919 | 0 | 0 | 0 |
| FCBBF3003800 | 0 | 5.547 | 0 | 0 | 1.390 | 0 | 1.373 |
| FCBBF3004473 | 0 | 0 | 8.428 | 0 | 0 | 0 | 0 |
| FCBBF3005320 | 0 | 0 | 0 | 0 | 0 | 0 | 9.510 |
| FCBBF3005330 | 0 | 0 | 2.541 | 0 | 2.444 | 0 | 0 |
| FCBBF3005497 | 0 | 0 | 0 | 0 | 8.322 | 0 | 0 |
| FCBBF3007829 | 0 | 79.216 | 0 | 0 | 0 | 0 | 0 |
| FCBBF3016134 | 0 | 0 | 0 | 0 | 0 | 0 | 36.291 |
| FCBBF3017396 | 0 | 0 | 0 | 0 | 7.204 | 56.122 | 0 |
| FCBBF3017531 | 0 | 0 | 0 | 0 | 0 | 44.077 | 0 |
| FCBBF3018796 | 0 | 0 | 0 | 0 | 0 | 0 | 10.823 |
| FCBBF3019714 | 0 | 0 | 7.513 | 0 | 0 | 0 | 0 |
| FCBBF3023667 | 11.655 | 0 | 0.487 | 0.993 | 0 | 0 | 0.463 |
| FCBBF3028244 | 0 | 0 | 0 | 0 | 3.276 | 0 | 0 |
| FCBBF4000446 | 0 | 5.289 | 0 | 0 | 0 | 0 | 0 |
| FEBRA1000088 | 0 | 0 | 0 | 0 | 0 | 0 | 14.878 |
| FEBRA2000105 | 0 | 0 | 2.631 | 0 | 0 | 0 | 15.003 |
| FEBRA2000129 | 0 | 0 | 0 | 0 | 0 | 0 | 9.063 |
| FEBRA2000210 | 0 | 0 | 0 | 0 | 0 | 0 | 4.929 |
| FEBRA2000228 | 0 | 0 | 14.488 | 0 | 0 | 0 | 0 |
| FEBRA2000510 | 0 | 0 | 0 | 0 | 13.019 | 0 | 0 |
| FEBRA2000536 | 0 | 0 | 0 | 23.543 | 0 | 0 | 0 |
| FEBRA2000733 | 0 | 7.120 | 1.236 | 1.260 | 0 | 0 | 0 |
| FEBRA2000881 | 0 | 0 | 11.610 | 23.659 | 11.169 | 0 | 0 |
| FEBRA2000901 | 0 | 0 | 0 | 0 | 36.652 | 0 | 0 |
| FEBRA2001383 | 0 | 0 | 0 | 0 | 36.809 | 0 | 0 |
| FEBRA2001814 | 0 | 0 | 0 | 0 | 0 | 0 | 9.225 |
| FEBRA2002027 | 0 | 0 | 9.292 | 0 | 0 | 0 | 0 |
| FEBRA2002707 | 0 | 0 | 0 | 26.932 | 0 | 0 | 0 |
| FEBRA2003100 | 0 | 4.637 | 0 | 0 | 0 | 0 | 0 |
| FEBRA2003926 | 0 | 0 | 2.560 | 0 | 0 | 0 | 4.867 |
| FEBRA2003930 | 0 | 0 | 0 | 0 | 6.342 | 0 | 0 |
| FEBRA2004224 | 0 | 0 | 0 | 8.653 | 0 | 0 | 0 |
| FEBRA2004325 | 0 | 0 | 0 | 0 | 0 | 0 | 12.279 |
| FEBRA2004412 | 0 | 0 | 10.768 | 0 | 0 | 0 | 0 |
| FEBRA2004818 | 0 | 0 | 24.972 | 0 | 0 | 0 | 0 |
| FEBRA2005752 | 0 | 29.308 | 0 | 0 | 0 | 0 | 0 |
| FEBRA2005778 | 0 | 0 | 11.002 | 0 | 0 | 0 | 0 |
| FEBRA2006055 | 0 | 18.599 | 0 | 0 | 0 | 0 | 0 |
| FEBRA2006092 | 0 | 0 | 0 | 0 | 2.194 | 0 | 0 |
| FEBRA2006370 | 0 | 0 | 0 | 0 | 0 | 0 | 49.808 |
| FEBRA2006372 | 0 | 0 | 0 | 0 | 4.739 | 0 | 0 |
| FEBRA2006485 | 0 | 0 | 11.157 | 0 | 0 | 0 | 0 |
| FEBRA2006553 | 0 | 27.857 | 0 | 0 | 0 | 0 | 0 |
| FEBRA2007801 | 0 | 17.382 | 0 | 0 | 0 | 0 | 0 |
| FEBRA2007880 | 0 | 0 | 13.804 | 0 | 0 | 0 | 0 |
| FEBRA2008036 | 0 | 0 | 0 | 0 | 5.040 | 0 | 0 |
| FEBRA2008081 | 0 | 4.986 | 0 | 0 | 0 | 0 | 0 |
| FEBRA2008087 | 15.732 | 0 | 1.315 | 0 | 1.265 | 0 | 0.625 |
| FEBRA2008255 | 0 | 0 | 18.560 | 0 | 0 | 0 | 0 |
| FEBRA2008302 | 0 | 7.493 | 0 | 0 | 0 | 0 | 0 |
| FEBRA2014417 | 0 | 0 | 1.980 | 12.107 | 0 | 0 | 0 |
| FEBRA2018476 | 0 | 0 | 0 | 0 | 0 | 0 | 3.809 |
| FEBRA2019711 | 0 | 0 | 7.756 | 0 | 0 | 0 | 0 |
| FEBRA2022055 | 46.701 | 0 | 1.952 | 7.954 | 0 | 0 | 0 |
| FEBRA2022504 | 0 | 0 | 0 | 12.276 | 0 | 0 | 0 |
| FEBRA2025838 | 0 | 5.784 | 0 | 0 | 0 | 22.582 | 2.864 |
| FEBRA2028516 | 0 | 0 | 4.195 | 0 | 0 | 31.443 | 0 |
| FELIV1000153 | 0 | 1.161 | 0 | 0 | 0 | 0 | 0 |
| HCASM2000016 | 0 | 0 | 0 | 5.578 | 0 | 0 | 0 |
| HCASM2000363 | 0 | 0 | 2.494 | 10.165 | 0 | 0 | 0 |
| HCASM2003076 | 0 | 0 | 0 | 0 | 0 | 0 | 19.851 |
| HCHON1000030 | 0 | 2.554 | 0.665 | 1.356 | 0 | 4.986 | 4.426 |
| HCHON1000191 | 0 | 3.401 | 0 | 7.220 | 0 | 0 | 0 |
| HCHON2000056 | 0 | 1.997 | 2.081 | 4.240 | 3.002 | 7.797 | 0.989 |
| HCHON2000087 | 0 | 0 | 4.538 | 0 | 0 | 0 | 0 |
| HCHON2000160 | 0 | 0 | 0 | 7.524 | 0 | 0 | 7.019 |
| HCHON2000226 | 0 | 0 | 2.958 | 0 | 0 | 0 | 0 |
| HCHON2000244 | 0 | 9.264 | 0 | 0 | 0 | 18.084 | 4.587 |
| HCHON2000265 | 0 | 0 | 0 | 0 | 0 | 0 | 7.054 |
| HCHON2000364 | 0 | 6.261 | 0 | 3.323 | 0 | 0 | 0 |
| HCHON2000475 | 0 | 0 | 0 | 9.949 | 0 | 0 | 0 |
| HCHON2000508 | 0 | 5.259 | 1.370 | 0 | 0 | 0 | 0 |
| HCHON2000626 | 0 | 8.277 | 0 | 0 | 0 | 0 | 0 |
| HCHON2000738 | 0 | 0 | 0 | 0 | 10.506 | 0 | 0 |
| HCHON2000826 | 0 | 5.039 | 0 | 0 | 0 | 0 | 0 |
| HCHON2000898 | 0 | 0 | 0 | 0 | 0 | 0 | 35.524 |
| HCHON2001039 | 0 | 0 | 0 | 0 | 0 | 0 | 3.890 |
| HCHON2001050 | 0 | 0 | 0 | 0 | 0 | 0 | 5.053 |
| HCHON2001108 | 0 | 0 | 7.832 | 0 | 0 | 0 | 0 |
| HCHON2001359 | 0 | 0 | 0 | 0 | 0 | 0 | 2.968 |
| HCHON2001407 | 0 | 0 | 0 | 0 | 2.786 | 0 | 0 |
| HCHON2001434 | 0 | 0 | 0 | 0 | 0 | 0 | 5.326 |
| HCHON2001505 | 0 | 0 | 0 | 0 | 15.856 | 0 | 0 |
| HCHON2001546 | 0 | 0 | 0 | 54.154 | 0 | 0 | 0 |
| HCHON2001665 | 0 | 0 | 0 | 0 | 0 | 0 | 5.380 |
| HCHON2004002 | 0 | 5.528 | 0 | 0 | 0 | 0 | 0 |
| HEART1000074 | 0 | 0 | 2.784 | 0 | 0 | 0 | 2.646 |
| HEART1000149 | 0 | 0 | 3.444 | 0 | 0 | 0 | 16.370 |
| HEART1000185 | 0 | 0 | 0 | 5.909 | 0 | 0 | 0 |
| HEART2003440 | 0 | 0 | 0 | 0 | 0 | 80.471 | 0 |
| HEART2004941 | 0 | 0 | 0 | 0 | 33.520 | 0 | 0 |
| HELAC2000158 | 100 | 0 | 0 | 0 | 0 | 0 | 0 |
| HELAC2000301 | 100 | 0 | 0 | 0 | 0 | 0 | 0 |
| HHDPC1000001 | 0 | 100 | 0 | 0 | 0 | 0 | 0 |
| HHDPC1000065 | 0 | 64.387 | 0 | 0 | 0 | 0 | 0 |
| HHDPC1000083 | 0 | 6.892 | 0 | 7.316 | 0 | 0 | 0 |
| HHDPC1000114 | 0 | 40.929 | 0 | 0 | 0 | 0 | 0 |
| HHDPC1000118 | 0 | 14.757 | 0 | 0 | 0 | 0 | 7.307 |
| HHDPC1000163 | 0 | 8.855 | 0 | 0 | 4.437 | 0 | 0 |
| HHDPC2000055 | 0 | 12.875 | 0 | 0 | 12.904 | 0 | 0 |
| HHDPC2000070 | 0 | 67.761 | 0 | 0 | 0 | 0 | 0 |
| HHDPC2000102 | 0 | 33.433 | 0 | 0 | 0 | 0 | 0 |
| HHDPC2000104 | 0 | 13.026 | 0 | 0 | 0 | 0 | 0 |
| HHDPC2000115 | 0 | 18.420 | 0 | 0 | 0 | 0 | 0 |
| HHDPC2000149 | 0 | 31.462 | 16.389 | 0 | 0 | 0 | 0 |
| HHDPC2000258 | 0 | 66.528 | 0 | 0 | 0 | 0 | 0 |
| HHDPC2000315 | 0 | 100 | 0 | 0 | 0 | 0 | 0 |
| HHDPC2000455 | 0 | 3.869 | 0 | 0 | 0 | 0 | 0 |
| HHDPC2000456 | 0 | 10.932 | 0 | 0 | 0 | 0 | 0 |
| HHDPC2000462 | 0 | 79.486 | 0 | 0 | 0 | 0 | 0 |
| HHDPC2000572 | 0 | 46.099 | 0 | 0 | 0 | 0 | 0 |
| HHDPC2000656 | 0 | 60.189 | 0 | 0 | 0 | 0 | 0 |
| HHDPC2000692 | 0 | 100 | 0 | 0 | 0 | 0 | 0 |
| HHDPC2001337 | 0 | 100 | 0 | 0 | 0 | 0 | 0 |
| HHDPC2001432 | 0 | 100 | 0 | 0 | 0 | 0 | 0 |
| HHDPC2001896 | 0 | 66.020 | 0 | 0 | 0 | 0 | 0 |
| HHDPC2002963 | 0 | 100 | 0 | 0 | 0 | 0 | 0 |
| HHDPC2003049 | 0 | 100 | 0 | 0 | 0 | 0 | 0 |
| HHDPC2003113 | 0 | 100 | 0 | 0 | 0 | 0 | 0 |
| HHDPC2003381 | 0 | 24.557 | 0 | 0 | 0 | 0 | 0 |
| HHDPC2003439 | 0 | 7.163 | 0.933 | 0 | 0 | 13.983 | 0 |
| HHDPC2003472 | 0 | 91.505 | 0 | 0 | 0 | 0 | 0 |
| HHDPC2003983 | 0 | 1.961 | 1.021 | 0 | 3.930 | 0 | 0 |
| HHDPC2004757 | 0 | 100 | 0 | 0 | 0 | 0 | 0 |
| HHDPC2005185 | 0 | 100 | 0 | 0 | 0 | 0 | 0 |
| HHDPC2005513 | 0 | 100 | 0 | 0 | 0 | 0 | 0 |
| HHDPC2005742 | 0 | 89.412 | 0 | 0 | 0 | 0 | 0 |
| HHDPC2005794 | 0 | 56.010 | 0 | 0 | 0 | 0 | 0 |
| HHDPC2006460 | 0 | 66.929 | 0 | 0 | 0 | 0 | 0 |
| HHDPC2006862 | 0 | 52.700 | 0 | 0 | 0 | 0 | 0 |
| HHDPC2006927 | 0 | 100 | 0 | 0 | 0 | 0 | 0 |
| HHDPC2007267 | 0 | 3.438 | 1.791 | 0 | 0 | 26.843 | 0 |
| HHDPC2007302 | 0 | 100 | 0 | 0 | 0 | 0 | 0 |
| HHDPC2007775 | 0 | 76.087 | 0 | 0 | 0 | 0 | 0 |
| HHDPC2008123 | 0 | 89.412 | 0 | 0 | 0 | 0 | 0 |
| HHDPC2008185 | 0 | 100 | 0 | 0 | 0 | 0 | 0 |
| HHDPC2008279 | 0 | 100 | 0 | 0 | 0 | 0 | 0 |
| HHDPC2008297 | 0 | 52.669 | 0 | 0 | 0 | 0 | 0 |
| HHDPC2008414 | 0 | 100 | 0 | 0 | 0 | 0 | 0 |
| HHDPC2008474 | 0 | 100 | 0 | 0 | 0 | 0 | 0 |
| HHDPC2008816 | 0 | 86.394 | 0 | 0 | 0 | 0 | 0 |
| HHDPC2008843 | 0 | 100 | 0 | 0 | 0 | 0 | 0 |
| HHDPC2009114 | 0 | 100 | 0 | 0 | 0 | 0 | 0 |
| HHDPC2009178 | 0 | 71.472 | 0 | 0 | 0 | 0 | 0 |
| HHDPC2009208 | 0 | 100 | 0 | 0 | 0 | 0 | 0 |
| HHDPC2009272 | 0 | 86.275 | 0 | 0 | 0 | 0 | 0 |
| HHDPC2009528 | 0 | 45.763 | 0 | 0 | 0 | 0 | 0 |
| HLUNG1000017 | 0 | 0 | 100 | 0 | 0 | 0 | 0 |
| HLUNG1000024 | 0 | 0 | 100 | 0 | 0 | 0 | 0 |
| HLUNG1000030 | 0 | 0 | 100 | 0 | 0 | 0 | 0 |
| HLUNG1000034 | 0 | 0 | 100 | 0 | 0 | 0 | 0 |
| HLUNG1000037 | 0 | 0 | 32.229 | 0 | 0 | 0 | 0 |
| HLUNG1000053 | 0 | 0 | 100 | 0 | 0 | 0 | 0 |
| HLUNG1000055 | 0 | 0 | 3.971 | 0 | 0 | 0 | 0 |
| HLUNG1000064 | 0 | 0 | 49.950 | 0 | 0 | 0 | 0 |
| HLUNG1000076 | 0 | 0 | 12.342 | 0 | 0 | 0 | 0 |
| HLUNG1000084 | 0 | 0 | 100 | 0 | 0 | 0 | 0 |
| HLUNG1000091 | 0 | 0 | 54.277 | 0 | 0 | 0 | 0 |
| HLUNG1000099 | 0 | 0 | 39.587 | 0 | 0 | 0 | 0 |
| HLUNG1000104 | 0 | 0 | 100 | 0 | 0 | 0 | 0 |
| HLUNG1000105 | 0 | 0 | 38.763 | 0 | 0 | 0 | 0 |
| HLUNG1000110 | 0 | 0 | 84.874 | 0 | 0 | 0 | 0 |
| HLUNG1000151 | 0 | 0 | 100 | 0 | 0 | 0 | 0 |
| HLUNG1000169 | 0 | 0 | 100 | 0 | 0 | 0 | 0 |
| HLUNG2000004 | 0 | 0 | 46.386 | 0 | 0 | 0 | 0 |
| HLUNG2000014 | 0 | 0 | 100 | 0 | 0 | 0 | 0 |
| HLUNG2000027 | 0 | 0 | 100 | 0 | 0 | 0 | 0 |
| HLUNG2000063 | 0 | 0 | 18.092 | 0 | 0 | 0 | 0 |
| HLUNG2000068 | 0 | 0 | 100 | 0 | 0 | 0 | 0 |
| HLUNG2000116 | 0 | 0 | 100 | 0 | 0 | 0 | 0 |
| HLUNG2000125 | 0 | 0 | 26.171 | 0 | 0 | 0 | 0 |
| HLUNG2000130 | 0 | 0 | 100 | 0 | 0 | 0 | 0 |
| HLUNG2000142 | 0 | 0 | 100 | 0 | 0 | 0 | 0 |
| HLUNG2000174 | 0 | 0 | 100 | 0 | 0 | 0 | 0 |
| HLUNG2000176 | 0 | 0 | 81.477 | 0 | 0 | 0 | 0 |
| HLUNG2000217 | 0 | 0 | 100 | 0 | 0 | 0 | 0 |
| HLUNG2000232 | 0 | 0 | 46.224 | 0 | 0 | 0 | 0 |
| HLUNG2000255 | 0 | 0 | 100 | 0 | 0 | 0 | 0 |
| HLUNG2000281 | 0 | 0 | 100 | 0 | 0 | 0 | 0 |
| HLUNG2000314 | 0 | 0 | 8.998 | 0 | 0 | 0 | 0 |
| HLUNG2000315 | 0 | 0 | 55.942 | 0 | 0 | 0 | 0 |
| HLUNG2000340 | 0 | 0 | 100 | 0 | 0 | 0 | 0 |
| HLUNG2000362 | 0 | 0 | 100 | 0 | 0 | 0 | 0 |
| HLUNG2000363 | 0 | 0 | 100 | 0 | 0 | 0 | 0 |
| HLUNG2000412 | 0 | 0 | 9.821 | 0 | 0 | 0 | 0 |
| HLUNG2000431 | 0 | 0 | 100 | 0 | 0 | 0 | 0 |
| HLUNG2000480 | 0 | 0 | 100 | 0 | 0 | 0 | 0 |
| HLUNG2000489 | 0 | 0 | 100 | 0 | 0 | 0 | 0 |
| HLUNG2000501 | 0 | 0 | 6.293 | 0 | 0 | 0 | 0 |
| HLUNG2000549 | 0 | 0 | 100 | 0 | 0 | 0 | 0 |
| HLUNG2000557 | 0 | 0 | 100 | 0 | 0 | 0 | 0 |
| HLUNG2000570 | 0 | 0 | 100 | 0 | 0 | 0 | 0 |
| HLUNG2000751 | 0 | 0 | 63.370 | 0 | 0 | 0 | 0 |
| HLUNG2000761 | 0 | 0 | 100 | 0 | 0 | 0 | 0 |
| HLUNG2000777 | 0 | 0 | 100 | 0 | 0 | 0 | 0 |
| HLUNG2000846 | 0 | 0 | 16.570 | 0 | 0 | 0 | 0 |
| HLUNG2000870 | 0 | 0 | 100 | 0 | 0 | 0 | 0 |
| HLUNG2000884 | 0 | 0 | 7.851 | 0 | 0 | 0 | 0 |
| HLUNG2000926 | 0 | 0 | 100 | 0 | 0 | 0 | 0 |
| HLUNG2000950 | 0 | 0 | 45.344 | 0 | 0 | 0 | 0 |
| HLUNG2000955 | 0 | 0 | 100 | 0 | 0 | 0 | 0 |
| HLUNG2000970 | 0 | 0 | 100 | 0 | 0 | 0 | 0 |
| HLUNG2001013 | 0 | 0 | 100 | 0 | 0 | 0 | 0 |
| HLUNG2001126 | 0 | 0 | 100 | 0 | 0 | 0 | 0 |
| HLUNG2001144 | 0 | 0 | 100 | 0 | 0 | 0 | 0 |
| HLUNG2001146 | 0 | 0 | 19.226 | 0 | 0 | 0 | 0 |
| HLUNG2001214 | 0 | 0 | 100 | 0 | 0 | 0 | 0 |
| HLUNG2001439 | 0 | 0 | 3.235 | 0 | 0 | 0 | 0 |
| HLUNG2001459 | 0 | 0 | 24.038 | 0 | 0 | 0 | 0 |
| HLUNG2001507 | 0 | 0 | 18.366 | 0 | 0 | 0 | 0 |
| HLUNG2001518 | 0 | 0 | 4.011 | 0 | 0 | 0 | 0 |
| HLUNG2001633 | 0 | 0 | 29.367 | 0 | 0 | 0 | 0 |
| HLUNG2001677 | 0 | 0 | 12.888 | 0 | 0 | 0 | 0 |
| HLUNG2001712 | 0 | 0 | 12.093 | 0 | 11.634 | 0 | 0 |
| HLUNG2001814 | 0 | 0 | 100 | 0 | 0 | 0 | 0 |
| HLUNG2001996 | 0 | 0 | 76.784 | 0 | 0 | 0 | 0 |
| HLUNG2002006 | 0 | 0 | 100 | 0 | 0 | 0 | 0 |
| HLUNG2002050 | 0 | 0 | 100 | 0 | 0 | 0 | 0 |
| HLUNG2002059 | 0 | 0 | 10.047 | 0 | 0 | 0 | 0 |
| HLUNG2002085 | 0 | 0 | 100 | 0 | 0 | 0 | 0 |
| HLUNG2002145 | 0 | 0 | 50.966 | 0 | 0 | 0 | 0 |
| HLUNG2002295 | 0 | 0 | 100 | 0 | 0 | 0 | 0 |
| HLUNG2002303 | 0 | 0 | 100 | 0 | 0 | 0 | 0 |
| HLUNG2002334 | 0 | 0 | 33.714 | 0 | 0 | 0 | 0 |
| HLUNG2002405 | 0 | 0 | 100 | 0 | 0 | 0 | 0 |
| HLUNG2002429 | 0 | 0 | 100 | 0 | 0 | 0 | 0 |
| HLUNG2002465 | 0 | 0 | 4.436 | 0 | 0 | 0 | 0 |
| HLUNG2002562 | 0 | 0 | 100 | 0 | 0 | 0 | 0 |
| HLUNG2002648 | 0 | 0 | 13.257 | 0 | 0 | 0 | 0 |
| HLUNG2002659 | 0 | 0 | 100 | 0 | 0 | 0 | 0 |
| HLUNG2002707 | 0 | 0 | 100 | 0 | 0 | 0 | 0 |
| HLUNG2002811 | 0 | 0 | 17.874 | 0 | 0 | 0 | 0 |
| HLUNG2002916 | 0 | 0 | 31.642 | 0 | 0 | 0 | 0 |
| HLUNG2002942 | 0 | 0 | 100 | 0 | 0 | 0 | 0 |
| HLUNG2002949 | 0 | 0 | 67.829 | 0 | 0 | 0 | 0 |
| HLUNG2002958 | 0 | 0 | 100 | 0 | 0 | 0 | 0 |
| HLUNG2002982 | 0 | 0 | 100 | 0 | 0 | 0 | 0 |
| HLUNG2003003 | 0 | 0 | 29.311 | 0 | 0 | 0 | 0 |
| HLUNG2003042 | 0 | 0 | 43.654 | 0 | 0 | 0 | 0 |
| HLUNG2003049 | 0 | 0 | 100 | 0 | 0 | 0 | 0 |
| HLUNG2003061 | 0 | 0 | 100 | 0 | 0 | 0 | 0 |
| HLUNG2003162 | 0 | 0 | 100 | 0 | 0 | 0 | 0 |
| HLUNG2003246 | 0 | 0 | 100 | 0 | 0 | 0 | 0 |
| HLUNG2003306 | 0 | 0 | 46.963 | 0 | 0 | 0 | 0 |
| HLUNG2003331 | 0 | 0 | 30.365 | 0 | 0 | 0 | 0 |
| HLUNG2003335 | 0 | 0 | 100 | 0 | 0 | 0 | 0 |
| HLUNG2003378 | 0 | 0 | 33.957 | 0 | 0 | 0 | 32.277 |
| HLUNG2003497 | 0 | 0 | 81.477 | 0 | 0 | 0 | 0 |
| HLUNG2003691 | 0 | 0 | 100 | 0 | 0 | 0 | 0 |
| HLUNG2003710 | 0 | 0 | 100 | 0 | 0 | 0 | 0 |
| HLUNG2003714 | 0 | 0 | 100 | 0 | 0 | 0 | 0 |
| HLUNG2003716 | 0 | 0 | 83.786 | 0 | 0 | 0 | 0 |
| HLUNG2003778 | 0 | 0 | 18.920 | 0 | 0 | 0 | 0 |
| HLUNG2003833 | 0 | 0 | 100 | 0 | 0 | 0 | 0 |
| HLUNG2003872 | 0 | 0 | 100 | 0 | 0 | 0 | 0 |
| HLUNG2003918 | 0 | 0 | 100 | 0 | 0 | 0 | 0 |
| HLUNG2003991 | 0 | 0 | 100 | 0 | 0 | 0 | 0 |
| HLUNG2004014 | 0 | 0 | 100 | 0 | 0 | 0 | 0 |
| HLUNG2004037 | 0 | 0 | 55.992 | 0 | 0 | 0 | 0 |
| HLUNG2004067 | 0 | 0 | 40.386 | 0 | 0 | 0 | 0 |
| HLUNG2004154 | 0 | 0 | 38.142 | 0 | 0 | 0 | 0 |
| HLUNG2004159 | 0 | 0 | 6.650 | 0 | 0 | 0 | 6.321 |
| HLUNG2004170 | 0 | 0 | 22.600 | 0 | 0 | 0 | 0 |
| HLUNG2004217 | 0 | 0 | 100 | 0 | 0 | 0 | 0 |
| HLUNG2004273 | 0 | 0 | 100 | 0 | 0 | 0 | 0 |
| HLUNG2004388 | 0 | 0 | 32.754 | 0 | 0 | 0 | 0 |
| HLUNG2004416 | 0 | 0 | 100 | 0 | 0 | 0 | 0 |
| HLUNG2004521 | 0 | 0 | 100 | 0 | 0 | 0 | 0 |
| HLUNG2004534 | 0 | 0 | 10.639 | 0 | 0 | 0 | 0 |
| HLUNG2004684 | 0 | 0 | 24.764 | 0 | 0 | 0 | 0 |
| HLUNG2004707 | 0 | 0 | 100 | 0 | 0 | 0 | 0 |
| HLUNG2004774 | 0 | 0 | 59.535 | 0 | 0 | 0 | 0 |
| HLUNG2004775 | 0 | 0 | 100 | 0 | 0 | 0 | 0 |
| HLUNG2005012 | 0 | 0 | 100 | 0 | 0 | 0 | 0 |
| HLUNG2005030 | 0 | 0 | 100 | 0 | 0 | 0 | 0 |
| HLUNG2005045 | 0 | 0 | 100 | 0 | 0 | 0 | 0 |
| HLUNG2005076 | 0 | 0 | 100 | 0 | 0 | 0 | 0 |
| HLUNG2005133 | 0 | 0 | 51.963 | 0 | 0 | 0 | 0 |
| HLUNG2005203 | 0 | 0 | 76.605 | 0 | 0 | 0 | 0 |
| HLUNG2005230 | 0 | 0 | 8.384 | 0 | 0 | 0 | 0 |
| HLUNG2005479 | 0 | 0 | 100 | 0 | 0 | 0 | 0 |
| HLUNG2005524 | 0 | 0 | 100 | 0 | 0 | 0 | 0 |
| HLUNG2005656 | 0 | 0 | 83.786 | 0 | 0 | 0 | 0 |
| HLUNG2005738 | 0 | 0 | 67.330 | 0 | 0 | 0 | 0 |
| HLUNG2005774 | 0 | 0 | 100 | 0 | 0 | 0 | 0 |
| HLUNG2005813 | 0 | 0 | 100 | 0 | 0 | 0 | 0 |
| HLUNG2005924 | 0 | 0 | 100 | 0 | 0 | 0 | 0 |
| HLUNG2006026 | 0 | 0 | 100 | 0 | 0 | 0 | 0 |
| HLUNG2006067 | 0 | 0 | 100 | 0 | 0 | 0 | 0 |
| HLUNG2006094 | 0 | 0 | 100 | 0 | 0 | 0 | 0 |
| HLUNG2006370 | 0 | 0 | 89.793 | 0 | 0 | 0 | 0 |
| HLUNG2006397 | 0 | 0 | 100 | 0 | 0 | 0 | 0 |
| HLUNG2006570 | 0 | 0 | 71.114 | 0 | 0 | 0 | 0 |
| HLUNG2006599 | 0 | 0 | 100 | 0 | 0 | 0 | 0 |
| HLUNG2006614 | 0 | 0 | 100 | 0 | 0 | 0 | 0 |
| HLUNG2006671 | 0 | 0 | 100 | 0 | 0 | 0 | 0 |
| HLUNG2006781 | 0 | 0 | 100 | 0 | 0 | 0 | 0 |
| HLUNG2006812 | 0 | 0 | 21.691 | 0 | 0 | 0 | 10.309 |
| HLUNG2006843 | 0 | 0 | 38.068 | 0 | 0 | 0 | 0 |
| HLUNG2006852 | 0 | 0 | 100 | 0 | 0 | 0 | 0 |
| HLUNG2006935 | 0 | 0 | 100 | 0 | 0 | 0 | 0 |
| HLUNG2007041 | 0 | 0 | 100 | 0 | 0 | 0 | 0 |
| HLUNG2007163 | 0 | 0 | 49.414 | 0 | 0 | 0 | 0 |
| HLUNG2007210 | 0 | 0 | 7.539 | 0 | 0 | 0 | 0 |
| HLUNG2007219 | 0 | 0 | 81.477 | 0 | 0 | 0 | 0 |
| HLUNG2007245 | 0 | 0 | 12.742 | 0 | 0 | 0 | 12.111 |
| HLUNG2007433 | 0 | 0 | 100 | 0 | 0 | 0 | 0 |
| HLUNG2007931 | 0 | 0 | 100 | 0 | 0 | 0 | 0 |
| HLUNG2008066 | 0 | 0 | 100 | 0 | 0 | 0 | 0 |
| HLUNG2008139 | 0 | 0 | 42.036 | 0 | 0 | 0 | 0 |
| HLUNG2008153 | 0 | 0 | 24.966 | 0 | 0 | 0 | 0 |
| HLUNG2008225 | 0 | 0 | 60.513 | 0 | 0 | 0 | 0 |
| HLUNG2008235 | 0 | 0 | 100 | 0 | 0 | 0 | 0 |
| HLUNG2008247 | 0 | 0 | 100 | 0 | 0 | 0 | 0 |
| HLUNG2008333 | 0 | 0 | 75.461 | 0 | 0 | 0 | 0 |
| HLUNG2008348 | 0 | 0 | 46.606 | 0 | 0 | 0 | 0 |
| HLUNG2008384 | 0 | 0 | 100 | 0 | 0 | 0 | 0 |
| HLUNG2008396 | 0 | 0 | 100 | 0 | 0 | 0 | 0 |
| HLUNG2008439 | 0 | 0 | 100 | 0 | 0 | 0 | 0 |
| HLUNG2008479 | 0 | 0 | 66.321 | 0 | 0 | 0 | 0 |
| HLUNG2008480 | 0 | 0 | 100 | 0 | 0 | 0 | 0 |
| HLUNG2008521 | 0 | 0 | 100 | 0 | 0 | 0 | 0 |
| HLUNG2008637 | 0 | 0 | 6.196 | 0 | 5.960 | 0 | 0 |
| HLUNG2008833 | 0 | 0 | 100 | 0 | 0 | 0 | 0 |
| HLUNG2008875 | 0 | 0 | 100 | 0 | 0 | 0 | 0 |
| HLUNG2008885 | 0 | 0 | 39.593 | 0 | 0 | 0 | 0 |
| HLUNG2008958 | 0 | 0 | 100 | 0 | 0 | 0 | 0 |
| HLUNG2008959 | 0 | 0 | 100 | 0 | 0 | 0 | 0 |
| HLUNG2009067 | 0 | 0 | 76.784 | 0 | 0 | 0 | 0 |
| HLUNG2009215 | 0 | 0 | 50.841 | 0 | 0 | 0 | 0 |
| HLUNG2009225 | 0 | 0 | 5.531 | 0 | 0 | 0 | 0 |
| HLUNG2009253 | 0 | 0 | 100 | 0 | 0 | 0 | 0 |
| HLUNG2009303 | 0 | 0 | 100 | 0 | 0 | 0 | 0 |
| HLUNG2009413 | 0 | 0 | 100 | 0 | 0 | 0 | 0 |
| HLUNG2009729 | 0 | 0 | 100 | 0 | 0 | 0 | 0 |
| HLUNG2010181 | 0 | 0 | 100 | 0 | 0 | 0 | 0 |
| HLUNG2010464 | 0 | 0 | 100 | 0 | 0 | 0 | 0 |
| HLUNG2010562 | 0 | 0 | 100 | 0 | 0 | 0 | 0 |
| HLUNG2010845 | 0 | 0 | 100 | 0 | 0 | 0 | 0 |
| HLUNG2011041 | 0 | 0 | 100 | 0 | 0 | 0 | 0 |
| HLUNG2011298 | 0 | 0 | 35.254 | 0 | 0 | 0 | 0 |
| HLUNG2011461 | 0 | 0 | 78.577 | 0 | 0 | 0 | 0 |
| HLUNG2011833 | 0 | 0 | 15.055 | 0 | 0 | 0 | 0 |
| HLUNG2012049 | 0 | 0 | 100 | 0 | 0 | 0 | 0 |
| HLUNG2012287 | 0 | 0 | 100 | 0 | 0 | 0 | 0 |
| HLUNG2012315 | 0 | 0 | 100 | 0 | 0 | 0 | 0 |
| HLUNG2012546 | 0 | 0 | 100 | 0 | 0 | 0 | 0 |
| HLUNG2012600 | 0 | 0 | 100 | 0 | 0 | 0 | 0 |
| HLUNG2012727 | 0 | 0 | 100 | 0 | 0 | 0 | 0 |
| HLUNG2013097 | 0 | 0 | 100 | 0 | 0 | 0 | 0 |
| HLUNG2013204 | 0 | 0 | 100 | 0 | 0 | 0 | 0 |
| HLUNG2013304 | 0 | 0 | 100 | 0 | 0 | 0 | 0 |
| HLUNG2013350 | 0 | 0 | 100 | 0 | 0 | 0 | 0 |
| HLUNG2013622 | 0 | 0 | 100 | 0 | 0 | 0 | 0 |
| HLUNG2013643 | 0 | 0 | 100 | 0 | 0 | 0 | 0 |
| HLUNG2013784 | 0 | 0 | 12.816 | 0 | 0 | 0 | 0 |
| HLUNG2013851 | 0 | 0 | 100 | 0 | 0 | 0 | 0 |
| HLUNG2014262 | 0 | 0 | 100 | 0 | 0 | 0 | 0 |
| HLUNG2014288 | 0 | 0 | 100 | 0 | 0 | 0 | 0 |
| HLUNG2014449 | 0 | 0 | 100 | 0 | 0 | 0 | 0 |
| HLUNG2014821 | 0 | 0 | 62.081 | 0 | 0 | 0 | 0 |
| HLUNG2015184 | 0 | 0 | 48.026 | 0 | 0 | 0 | 0 |
| HLUNG2015418 | 0 | 0 | 76.605 | 0 | 0 | 0 | 0 |
| HLUNG2015548 | 0 | 0 | 100 | 0 | 0 | 0 | 0 |
| HLUNG2015578 | 0 | 0 | 100 | 0 | 0 | 0 | 0 |
| HLUNG2015617 | 0 | 0 | 100 | 0 | 0 | 0 | 0 |
| HLUNG2016022 | 0 | 0 | 100 | 0 | 0 | 0 | 0 |
| HLUNG2016862 | 0 | 0 | 100 | 0 | 0 | 0 | 0 |
| HLUNG2017262 | 0 | 0 | 5.816 | 0 | 0 | 0 | 0 |
| HLUNG2017286 | 0 | 0 | 100 | 0 | 0 | 0 | 0 |
| HLUNG2017307 | 0 | 0 | 100 | 0 | 0 | 0 | 0 |
| HLUNG2017350 | 0 | 0 | 100 | 0 | 0 | 0 | 0 |
| HLUNG2017546 | 0 | 0 | 12.944 | 0 | 0 | 0 | 0 |
| HLUNG2017806 | 0 | 0 | 100 | 0 | 0 | 0 | 0 |
| HLUNG2018029 | 0 | 0 | 100 | 0 | 0 | 0 | 0 |
| HLUNG2018282 | 0 | 0 | 100 | 0 | 0 | 0 | 0 |
| HLUNG2018626 | 0 | 0 | 100 | 0 | 0 | 0 | 0 |
| HLUNG2019058 | 0 | 0 | 100 | 0 | 0 | 0 | 0 |
| HSYRA1000062 | 0 | 3.016 | 1.571 | 3.201 | 1.511 | 0 | 0 |
| HSYRA1000119 | 0 | 3.237 | 1.686 | 3.437 | 0 | 0 | 4.809 |
| HSYRA1000131 | 0 | 0 | 0 | 100 | 0 | 0 | 0 |
| HSYRA1000137 | 0 | 0 | 0 | 27.855 | 13.150 | 0 | 0 |
| HSYRA1000148 | 0 | 0 | 0 | 49.288 | 0 | 0 | 0 |
| HSYRA1000152 | 0 | 0 | 0 | 8.915 | 0 | 0 | 4.158 |
| HSYRA1000178 | 0 | 0 | 9.197 | 18.741 | 0 | 0 | 0 |
| HSYRA2000044 | 0 | 0 | 0 | 89.963 | 0 | 0 | 0 |
| HSYRA2000135 | 0 | 27.849 | 0 | 29.561 | 0 | 0 | 0 |
| HSYRA2000137 | 0 | 0 | 0 | 16.941 | 0 | 0 | 0 |
| HSYRA2000159 | 0 | 0.849 | 0 | 0.901 | 0 | 0 | 0.420 |
| HSYRA2000190 | 0 | 0 | 0 | 17.752 | 8.381 | 0 | 0 |
| HSYRA2000221 | 0 | 0 | 0 | 70.752 | 0 | 0 | 0 |
| HSYRA2000224 | 0 | 0 | 6.595 | 13.440 | 0 | 0 | 0 |
| HSYRA2000232 | 0 | 0 | 0 | 5.527 | 0 | 0 | 2.578 |
| HSYRA2000237 | 0 | 0 | 0 | 100 | 0 | 0 | 0 |
| HSYRA2000248 | 0 | 0 | 0 | 15.240 | 0 | 0 | 0 |
| HSYRA2000253 | 0 | 0 | 0 | 63.145 | 29.810 | 0 | 0 |
| HSYRA2000255 | 0 | 0 | 0 | 3.354 | 0 | 0 | 0 |
| HSYRA2000332 | 0 | 0 | 2.861 | 29.148 | 0 | 0 | 0 |
| HSYRA2000371 | 0 | 0 | 0 | 100 | 0 | 0 | 0 |
| HSYRA2000424 | 0 | 0 | 0 | 86.967 | 0 | 0 | 0 |
| HSYRA2000507 | 0 | 0 | 0 | 91.958 | 0 | 0 | 0 |
| HSYRA2000510 | 0 | 0 | 0 | 100 | 0 | 0 | 0 |
| HSYRA2000605 | 0 | 0 | 0 | 44.719 | 0 | 0 | 0 |
| HSYRA2000629 | 0 | 0 | 0 | 74.837 | 0 | 0 | 0 |
| HSYRA2000640 | 0 | 5.999 | 0 | 6.367 | 6.012 | 0 | 0 |
| HSYRA2000706 | 0 | 0 | 0 | 91.327 | 0 | 0 | 0 |
| HSYRA2000743 | 0 | 0 | 0 | 5.784 | 0 | 0 | 0 |
| HSYRA2000760 | 0 | 0 | 0 | 100 | 0 | 0 | 0 |
| HSYRA2000787 | 0 | 0 | 0 | 23.247 | 0 | 0 | 0 |
| HSYRA2000792 | 0 | 0 | 0 | 18.101 | 0 | 66.575 | 0 |
| HSYRA2000828 | 0 | 0 | 0 | 21.296 | 0 | 0 | 0 |
| HSYRA2000832 | 0 | 0 | 32.918 | 67.082 | 0 | 0 | 0 |
| HSYRA2000925 | 0 | 1.560 | 0 | 1.656 | 0.782 | 0 | 0 |
| HSYRA2000927 | 0 | 0 | 0 | 100 | 0 | 0 | 0 |
| HSYRA2001003 | 0 | 0 | 0 | 64.164 | 0 | 0 | 0 |
| HSYRA2001103 | 0 | 17.916 | 0 | 6.339 | 0 | 0 | 0 |
| HSYRA2001105 | 0 | 0 | 0 | 20.593 | 0 | 0 | 0 |
| HSYRA2001138 | 0 | 5.385 | 1.870 | 11.433 | 0.900 | 0 | 5.333 |
| HSYRA2001142 | 0 | 0 | 0 | 100 | 0 | 0 | 0 |
| HSYRA2001153 | 0 | 0 | 0 | 68.327 | 0 | 0 | 0 |
| HSYRA2001225 | 0 | 0 | 0 | 5.525 | 0 | 0 | 0 |
| HSYRA2001232 | 0 | 0 | 0 | 57.829 | 0 | 0 | 0 |
| HSYRA2001255 | 0 | 0 | 0 | 100 | 0 | 0 | 0 |
| HSYRA2001332 | 0 | 0 | 0 | 100 | 0 | 0 | 0 |
| HSYRA2001353 | 0 | 0 | 0 | 37.924 | 0 | 0 | 0 |
| HSYRA2001396 | 0 | 0 | 0 | 11.355 | 0 | 0 | 0 |
| HSYRA2001420 | 0 | 0 | 20.436 | 41.645 | 0 | 0 | 0 |
| HSYRA2001476 | 0 | 3.399 | 0 | 3.608 | 0 | 0 | 0 |
| HSYRA2001484 | 0 | 0 | 0 | 67.457 | 0 | 0 | 0 |
| HSYRA2001552 | 0 | 0 | 0 | 100 | 0 | 0 | 0 |
| HSYRA2001567 | 0 | 0 | 0 | 33.500 | 0 | 0 | 0 |
| HSYRA2001574 | 0 | 0 | 0 | 5.624 | 0 | 0 | 0 |
| HSYRA2001580 | 0 | 0 | 0 | 40.964 | 0 | 0 | 0 |
| HSYRA2001595 | 0 | 0 | 2.089 | 4.258 | 0 | 0 | 0 |
| HSYRA2001615 | 0 | 0 | 0 | 15.501 | 0 | 0 | 0 |
| HSYRA2001621 | 0 | 0 | 0 | 1.371 | 0 | 0 | 0 |
| HSYRA2001631 | 0 | 0 | 0 | 43.582 | 0 | 0 | 0 |
| HSYRA2001638 | 0 | 0 | 0 | 16.905 | 0 | 0 | 0 |
| HSYRA2001983 | 0 | 0 | 0 | 36.074 | 0 | 0 | 16.826 |
| HSYRA2002103 | 0 | 0 | 0 | 100 | 0 | 0 | 0 |
| HSYRA2002590 | 0 | 0 | 0 | 100 | 0 | 0 | 0 |
| HSYRA2002741 | 0 | 0 | 0 | 52.597 | 0 | 0 | 0 |
| HSYRA2003168 | 0 | 0 | 0 | 100 | 0 | 0 | 0 |
| HSYRA2004550 | 0 | 0 | 0 | 100 | 0 | 0 | 0 |
| HSYRA2004858 | 0 | 0 | 0 | 100 | 0 | 0 | 0 |
| HSYRA2005456 | 0 | 0 | 0 | 100 | 0 | 0 | 0 |
| HSYRA2005496 | 0 | 0 | 0 | 100 | 0 | 0 | 0 |
| HSYRA2005628 | 0 | 0 | 0 | 50.126 | 0 | 0 | 0 |
| HSYRA2005970 | 0 | 0 | 0 | 100 | 0 | 0 | 0 |
| HSYRA2006049 | 0 | 0 | 0 | 50.609 | 0 | 0 | 0 |
| HSYRA2006873 | 0 | 0 | 0 | 100 | 0 | 0 | 0 |
| HSYRA2007241 | 0 | 0 | 0 | 57.334 | 0 | 0 | 0 |
| HSYRA2007338 | 0 | 0 | 0 | 100 | 0 | 0 | 0 |
| HSYRA2007650 | 0 | 0 | 0 | 35.551 | 0 | 0 | 0 |
| HSYRA2007667 | 0 | 0 | 0 | 66.840 | 0 | 0 | 0 |
| HSYRA2008154 | 0 | 0 | 0 | 100 | 0 | 0 | 0 |
| HSYRA2008714 | 0 | 0 | 0 | 100 | 0 | 0 | 0 |
| HSYRA2009102 | 0 | 0 | 0 | 100 | 0 | 0 | 0 |
| IMR321000155 | 0 | 0 | 0 | 0 | 100 | 0 | 0 |
| IMR321000158 | 0 | 5.360 | 0 | 0 | 13.429 | 0 | 0 |
| IMR321000161 | 0 | 0 | 0 | 0 | 80.886 | 0 | 0 |
| IMR321000165 | 0 | 0 | 0 | 0 | 100 | 0 | 0 |
| IMR321000193 | 0 | 0 | 0 | 0 | 15.487 | 0 | 15.302 |
| IMR321000207 | 0 | 0 | 0 | 0 | 49.332 | 0 | 0 |
| IMR321000210 | 0 | 0 | 0 | 0 | 7.153 | 0 | 0 |
| IMR321000219 | 0 | 0 | 1.029 | 0 | 0.990 | 0 | 0 |
| IMR321000230 | 0 | 0 | 0 | 0 | 100 | 0 | 0 |
| IMR321000242 | 0 | 0 | 0 | 0 | 19.843 | 0 | 0 |
| IMR321000253 | 0 | 0 | 0 | 0 | 3.594 | 0 | 0 |
| IMR321000266 | 0 | 0 | 10.379 | 0 | 9.985 | 0 | 0 |
| IMR322000010 | 0 | 0 | 0 | 0 | 33.404 | 0 | 0 |
| IMR322000072 | 0 | 0 | 0 | 0 | 25.481 | 0 | 0 |
| IMR322000107 | 0 | 0 | 0 | 0 | 100 | 0 | 0 |
| IMR322000121 | 0 | 0 | 0 | 0 | 7.867 | 0 | 0 |
| IMR322000127 | 0 | 0 | 0 | 0 | 5.466 | 0 | 0 |
| IMR322000144 | 0 | 0 | 0 | 0 | 11.511 | 0 | 0 |
| IMR322000152 | 0 | 0 | 0 | 0 | 28.585 | 0 | 0 |
| IMR322000164 | 0 | 0 | 0 | 0 | 100 | 0 | 0 |
| IMR322000178 | 0 | 0 | 0 | 0 | 6.562 | 0 | 0 |
| IMR322000223 | 0 | 0 | 0 | 0 | 2.402 | 0 | 0 |
| IMR322000243 | 0 | 0 | 3.147 | 0 | 6.055 | 0 | 0 |
| IMR322000302 | 0 | 0 | 0 | 0 | 20.933 | 0 | 0 |
| IMR322000316 | 0 | 0 | 0 | 0 | 1.464 | 0 | 0 |
| IMR322000374 | 0 | 0 | 0 | 0 | 19.436 | 0 | 0 |
| IMR322000379 | 0 | 0 | 0 | 0 | 100 | 0 | 0 |
| IMR322000383 | 0 | 0 | 0 | 0 | 100 | 0 | 0 |
| IMR322000418 | 0 | 0 | 0 | 0 | 100 | 0 | 0 |
| IMR322000518 | 0 | 0 | 0 | 0 | 32.511 | 0 | 0 |
| IMR322000555 | 0 | 0 | 0 | 0 | 21.051 | 0 | 0 |
| IMR322000604 | 0 | 0 | 0 | 0 | 77.918 | 0 | 0 |
| IMR322000609 | 0 | 0 | 0 | 0 | 77.918 | 0 | 0 |
| IMR322000656 | 0 | 0 | 0 | 0 | 100 | 0 | 0 |
| IMR322000672 | 0 | 0 | 0 | 0 | 100 | 0 | 0 |
| IMR322000730 | 0 | 2.895 | 0.503 | 0 | 1.934 | 0 | 0 |
| IMR322000742 | 0 | 0 | 0 | 0 | 17.529 | 0 | 5.773 |
| IMR322000775 | 0 | 0 | 0 | 0 | 29.259 | 0 | 0 |
| IMR322000791 | 0 | 0 | 0 | 0 | 18.307 | 0 | 0 |
| IMR322000811 | 0 | 0 | 0 | 0 | 49.874 | 0 | 0 |
| IMR322000819 | 0 | 0 | 0 | 0 | 30.079 | 0 | 0 |
| IMR322000838 | 0 | 0 | 0 | 0 | 76.087 | 0 | 0 |
| IMR322000859 | 0 | 0 | 0 | 0 | 71.312 | 0 | 0 |
| IMR322000863 | 0 | 0 | 0 | 0 | 95.212 | 0 | 0 |
| IMR322000917 | 0 | 0 | 0 | 0 | 41.680 | 0 | 0 |
| IMR322000918 | 0 | 0 | 0 | 0 | 10.343 | 0 | 0 |
| IMR322000919 | 0 | 0 | 0 | 0 | 100 | 0 | 0 |
| IMR322000935 | 0 | 0 | 0 | 0 | 5.283 | 0 | 0 |
| IMR322000953 | 0 | 0 | 0 | 0 | 100 | 0 | 0 |
| IMR322000973 | 0 | 0 | 0 | 0 | 2.845 | 0 | 0 |
| IMR322000984 | 0 | 0 | 0 | 0 | 100 | 0 | 0 |
| IMR322001018 | 0 | 0 | 0 | 0 | 100 | 0 | 0 |
| IMR322001049 | 0 | 0 | 0 | 0 | 20.440 | 0 | 0 |
| IMR322001167 | 0 | 0 | 0 | 0 | 100 | 0 | 0 |
| IMR322001185 | 0 | 0 | 0 | 0 | 49.459 | 0 | 0 |
| IMR322001218 | 0 | 0 | 0 | 0 | 7.900 | 0 | 0 |
| IMR322001245 | 0 | 0 | 0 | 0 | 100 | 0 | 0 |
| IMR322001317 | 0 | 0 | 0 | 0 | 22.291 | 0 | 0 |
| IMR322001318 | 0 | 0 | 0 | 0 | 77.787 | 0 | 0 |
| IMR322001322 | 0 | 0 | 0 | 0 | 77.787 | 0 | 0 |
| IMR322001332 | 0 | 0 | 0 | 0 | 28.184 | 0 | 0 |
| IMR322001380 | 0 | 0 | 0 | 0 | 13.989 | 0 | 0 |
| IMR322001435 | 0 | 6.615 | 3.446 | 0 | 6.630 | 0 | 3.275 |
| IMR322001438 | 0 | 0 | 0 | 0 | 29.322 | 0 | 0 |
| IMR322001452 | 0 | 0 | 0 | 0 | 100 | 0 | 0 |
| IMR322001491 | 0 | 0 | 0 | 0 | 34.025 | 0 | 0 |
| IMR322001534 | 0 | 0 | 0 | 0 | 29.156 | 0 | 0 |
| IMR322001541 | 0 | 0 | 0 | 0 | 47.754 | 0 | 0 |
| IMR322001584 | 0 | 0 | 0 | 0 | 4.405 | 34.316 | 0 |
| IMR322001600 | 0 | 1.134 | 0 | 0 | 0.568 | 0 | 0 |
| IMR322001665 | 0 | 0 | 0 | 0 | 100 | 0 | 0 |
| IMR322001670 | 0 | 0 | 0 | 0 | 16.098 | 0 | 0 |
| IMR322001710 | 0 | 0 | 0 | 0 | 13.687 | 0 | 0 |
| IMR322001724 | 0 | 0 | 0 | 0 | 100 | 0 | 0 |
| IMR322001879 | 0 | 0 | 0 | 0 | 34.007 | 0 | 0 |
| IMR322002035 | 0 | 0 | 0 | 0 | 63.824 | 0 | 0 |
| IMR322002110 | 0 | 0 | 0 | 0 | 100 | 0 | 0 |
| IMR322002470 | 0 | 0 | 0 | 0 | 100 | 0 | 0 |
| IMR322002993 | 0 | 0 | 0 | 0 | 54.923 | 0 | 0 |
| IMR322003675 | 0 | 0 | 0 | 0 | 100 | 0 | 0 |
| IMR322004768 | 0 | 0 | 0 | 0 | 100 | 0 | 0 |
| IMR322005293 | 0 | 0 | 0 | 0 | 100 | 0 | 0 |
| IMR322005833 | 0 | 0 | 0 | 0 | 100 | 0 | 0 |
| IMR322006012 | 0 | 0 | 0 | 0 | 100 | 0 | 0 |
| IMR322006222 | 0 | 0 | 0 | 0 | 100 | 0 | 0 |
| IMR322006495 | 0 | 0 | 0 | 0 | 100 | 0 | 0 |
| IMR322006520 | 0 | 0 | 0 | 0 | 100 | 0 | 0 |
| IMR322006886 | 0 | 0 | 0 | 0 | 100 | 0 | 0 |
| IMR322006947 | 0 | 0 | 0 | 0 | 32.603 | 0 | 0 |
| IMR322007078 | 0 | 0 | 0 | 0 | 100 | 0 | 0 |
| IMR322007225 | 0 | 0 | 0 | 0 | 100 | 0 | 0 |
| IMR322007704 | 0 | 0 | 0 | 0 | 100 | 0 | 0 |
| IMR322008651 | 0 | 0 | 0 | 0 | 100 | 0 | 0 |
| IMR322009710 | 0 | 0 | 0 | 0 | 83.254 | 0 | 0 |
| IMR322009807 | 0 | 0 | 0 | 0 | 100 | 0 | 0 |
| IMR322010295 | 0 | 0 | 0 | 0 | 100 | 0 | 0 |
| IMR322010953 | 0 | 0 | 0 | 0 | 100 | 0 | 0 |
| IMR322011689 | 0 | 0 | 0 | 0 | 100 | 0 | 0 |
| IMR322012314 | 0 | 0 | 0 | 0 | 100 | 0 | 0 |
| IMR322012529 | 0 | 0 | 0 | 0 | 16.616 | 0 | 0 |
| IMR322013053 | 0 | 0 | 0 | 0 | 100 | 0 | 0 |
| IMR322013396 | 0 | 0 | 0 | 0 | 100 | 0 | 0 |
| IMR322013731 | 0 | 0 | 0 | 0 | 100 | 0 | 0 |
| IMR322013805 | 0 | 0 | 0 | 0 | 47.665 | 0 | 0 |
| IMR322015523 | 0 | 0 | 0 | 0 | 100 | 0 | 0 |
| IMR322016146 | 0 | 0 | 0 | 0 | 100 | 0 | 0 |
| IMR322017049 | 0 | 0 | 0 | 0 | 100 | 0 | 0 |
| IMR322018117 | 0 | 0 | 0 | 0 | 100 | 0 | 0 |
| IMR322018192 | 0 | 0 | 0 | 0 | 100 | 0 | 0 |
| IMR322019070 | 0 | 0 | 9.813 | 0 | 9.441 | 0 | 0 |
| JCMLC2000273 | 0 | 0 | 0 | 0 | 0 | 84.957 | 0 |
| KIDNE1000008 | 0 | 0 | 0 | 0 | 0 | 0 | 56.319 |
| KIDNE1000012 | 0 | 0 | 0 | 0 | 0 | 0 | 100 |
| KIDNE1000015 | 0 | 0 | 0 | 0 | 0 | 0 | 100 |
| KIDNE1000028 | 0 | 0 | 0 | 0 | 0 | 0 | 100 |
| KIDNE1000036 | 0 | 0 | 0 | 0 | 0 | 0 | 74.510 |
| KIDNE1000064 | 0 | 0 | 0 | 0 | 0 | 0 | 100 |
| KIDNE1000104 | 0 | 0 | 0 | 0 | 0 | 0 | 57.817 |
| KIDNE1000121 | 0 | 0 | 0 | 0 | 0 | 0 | 100 |
| KIDNE1000143 | 0 | 0 | 0 | 0 | 0 | 0 | 55.638 |
| KIDNE1000145 | 0 | 0 | 0 | 0 | 0 | 0 | 9.641 |
| KIDNE1000152 | 0 | 0 | 0 | 0 | 0 | 0 | 100 |
| KIDNE2000026 | 0 | 0 | 0 | 0 | 0 | 0 | 100 |
| KIDNE2000041 | 0 | 0 | 0 | 0 | 0 | 0 | 100 |
| KIDNE2000046 | 0 | 0 | 0 | 0 | 0 | 0 | 100 |
| KIDNE2000051 | 0 | 0 | 0 | 0 | 0 | 0 | 100 |
| KIDNE2000070 | 0 | 0 | 0 | 0 | 0 | 0 | 100 |
| KIDNE2000085 | 0 | 0 | 0 | 0 | 0 | 0 | 7.170 |
| KIDNE2000167 | 0 | 0 | 0 | 0 | 0 | 0 | 100 |
| KIDNE2000174 | 0 | 0 | 0 | 0 | 0 | 0 | 100 |
| KIDNE2000192 | 0 | 0 | 0 | 0 | 0 | 0 | 100 |
| KIDNE2000227 | 0 | 0 | 0 | 0 | 0 | 0 | 100 |
| KIDNE2000244 | 0 | 0 | 0 | 0 | 0 | 0 | 100 |
| KIDNE2000245 | 0 | 0 | 0 | 0 | 0 | 0 | 100 |
| KIDNE2000252 | 0 | 0 | 0 | 6.388 | 0 | 0 | 2.979 |
| KIDNE2000266 | 0 | 0 | 15.443 | 0 | 0 | 0 | 14.679 |
| KIDNE2000287 | 0 | 0 | 0 | 0 | 0 | 0 | 100 |
| KIDNE2000315 | 0 | 0 | 0 | 0 | 0 | 0 | 47.614 |
| KIDNE2000317 | 0 | 0 | 0 | 0 | 0 | 0 | 49.600 |
| KIDNE2000328 | 0 | 0 | 0 | 0 | 0 | 0 | 100 |
| KIDNE2000330 | 0 | 0 | 0 | 0 | 0 | 0 | 11.902 |
| KIDNE2000335 | 0 | 0 | 0 | 0 | 0 | 0 | 100 |
| KIDNE2000349 | 0 | 0 | 0 | 0 | 0 | 52.159 | 30.868 |
| KIDNE2000375 | 0 | 0 | 0 | 0 | 0 | 0 | 100 |
| KIDNE2000383 | 0 | 0 | 0 | 0 | 0 | 0 | 17.169 |
| KIDNE2000394 | 0 | 0 | 0 | 0 | 0 | 0 | 100 |
| KIDNE2000403 | 0 | 0 | 0 | 0 | 0 | 0 | 2.880 |
| KIDNE2000422 | 0 | 0 | 0 | 0 | 0 | 0 | 77.710 |
| KIDNE2000493 | 0 | 0 | 0 | 0 | 0 | 0 | 100 |
| KIDNE2000497 | 0 | 0 | 0 | 0 | 0 | 0 | 10.932 |
| KIDNE2000510 | 0 | 0 | 0 | 0 | 0 | 0 | 100 |
| KIDNE2000513 | 0 | 0 | 0 | 0 | 0 | 0 | 100 |
| KIDNE2000517 | 0 | 0 | 0 | 0 | 0 | 0 | 27.300 |
| KIDNE2000519 | 0 | 0 | 0 | 0 | 0 | 0 | 100 |
| KIDNE2000555 | 0 | 0 | 0 | 0 | 0 | 0 | 100 |
| KIDNE2000574 | 0 | 5.271 | 0 | 0 | 2.641 | 0 | 2.610 |
| KIDNE2000624 | 0 | 0 | 0 | 0 | 0 | 0 | 100 |
| KIDNE2000665 | 0 | 0 | 0 | 0 | 0 | 0 | 13.498 |
| KIDNE2000678 | 0 | 0 | 0 | 0 | 0 | 0 | 100 |
| KIDNE2000717 | 0 | 0 | 0 | 0 | 0 | 0 | 100 |
| KIDNE2000721 | 0 | 0 | 0 | 0 | 0 | 0 | 73.728 |
| KIDNE2000722 | 0 | 0 | 0 | 0 | 0 | 0 | 100 |
| KIDNE2000777 | 0 | 0 | 0 | 0 | 0 | 0 | 19.958 |
| KIDNE2000801 | 0 | 0 | 0 | 0 | 0 | 0 | 100 |
| KIDNE2000832 | 0 | 0 | 0 | 0 | 0 | 0 | 32.592 |
| KIDNE2000833 | 0 | 0 | 0 | 0 | 0 | 0 | 25.115 |
| KIDNE2000846 | 0 | 0 | 0 | 0 | 0 | 0 | 100 |
| KIDNE2000867 | 0 | 0 | 0 | 0 | 0 | 0 | 100 |
| KIDNE2000909 | 0 | 0 | 0 | 0 | 0 | 0 | 7.157 |
| KIDNE2000947 | 0 | 0 | 0 | 0 | 0 | 0 | 8.811 |
| KIDNE2001117 | 0 | 0 | 0 | 0 | 0 | 0 | 77.710 |
| KIDNE2001140 | 0 | 0 | 0 | 0 | 0 | 0 | 100 |
| KIDNE2001162 | 0 | 0 | 0 | 3.489 | 1.647 | 0 | 1.627 |
| KIDNE2001269 | 0 | 0 | 0 | 0 | 0 | 0 | 100 |
| KIDNE2001361 | 0 | 0 | 0 | 0 | 0 | 0 | 80.699 |
| KIDNE2001373 | 0 | 0 | 0 | 0 | 0 | 0 | 24.839 |
| KIDNE2001467 | 0 | 0 | 0 | 0 | 0 | 7.165 | 0.909 |
| KIDNE2001636 | 0 | 0 | 0 | 0 | 0 | 0 | 49.423 |
| KIDNE2001713 | 0 | 0 | 0 | 0 | 0 | 0 | 54.019 |
| KIDNE2001802 | 0 | 0 | 0 | 0 | 0 | 0 | 100 |
| KIDNE2001840 | 0 | 0 | 0 | 0 | 0 | 0 | 100 |
| KIDNE2001847 | 0 | 0 | 0 | 0 | 0 | 0 | 12.687 |
| KIDNE2001857 | 0 | 0 | 0 | 0 | 0 | 0 | 100 |
| KIDNE2001873 | 0 | 0 | 0 | 0 | 0 | 0 | 100 |
| KIDNE2001897 | 0 | 0 | 0 | 0 | 0 | 0 | 100 |
| KIDNE2001931 | 0 | 0 | 0 | 0 | 0 | 0 | 100 |
| KIDNE2001979 | 0 | 0 | 0 | 44.035 | 0 | 0 | 20.540 |
| KIDNE2002015 | 0 | 0 | 0 | 0 | 0 | 0 | 100 |
| KIDNE2002162 | 0 | 0 | 0 | 0 | 0 | 0 | 100 |
| KIDNE2002168 | 0 | 0 | 0 | 0 | 0 | 0 | 100 |
| KIDNE2002191 | 0 | 0 | 0 | 0 | 0 | 0 | 24.850 |
| KIDNE2002198 | 0 | 0 | 0 | 0 | 0 | 0 | 100 |
| KIDNE2002252 | 0 | 0 | 0 | 0 | 0 | 0 | 7.600 |
| KIDNE2002262 | 0 | 0 | 0 | 0 | 0 | 0 | 100 |
| KIDNE2002265 | 0 | 0 | 0 | 0 | 0 | 0 | 6.341 |
| KIDNE2002438 | 0 | 0 | 0 | 0 | 0 | 0 | 65.363 |
| KIDNE2002483 | 0 | 0 | 0 | 0 | 0 | 0 | 100 |
| KIDNE2002725 | 0 | 0 | 0 | 0 | 0 | 0 | 35.870 |
| KIDNE2002768 | 0 | 0 | 0 | 0 | 0 | 0 | 100 |
| KIDNE2002795 | 0 | 0 | 0 | 0 | 0 | 0 | 100 |
| KIDNE2002798 | 0 | 0 | 44.388 | 0 | 0 | 0 | 42.192 |
| KIDNE2002839 | 0 | 0 | 0 | 0 | 0 | 0 | 25.593 |
| KIDNE2002845 | 0 | 0 | 0 | 0 | 0 | 0 | 100 |
| KIDNE2002872 | 0 | 0 | 0 | 0 | 0 | 0 | 1.993 |
| KIDNE2002882 | 0 | 0 | 0 | 0 | 0 | 0 | 28.683 |
| KIDNE2002883 | 0 | 0 | 0 | 0 | 0 | 0 | 7.907 |
| KIDNE2002980 | 0 | 0 | 0 | 0 | 0 | 0 | 10.988 |
| KIDNE2002991 | 0 | 0 | 0 | 0 | 0 | 0 | 100 |
| KIDNE2003185 | 0 | 0 | 0 | 0 | 0 | 0 | 100 |
| KIDNE2003188 | 0 | 0 | 0 | 0 | 0 | 0 | 100 |
| KIDNE2003233 | 0 | 0 | 0 | 0 | 0 | 0 | 83.085 |
| KIDNE2003305 | 0 | 0 | 6.174 | 0 | 0 | 0 | 8.803 |
| KIDNE2003316 | 0 | 0 | 0 | 0 | 0 | 0 | 26.377 |
| KIDNE2003335 | 0 | 0 | 0 | 0 | 0 | 0 | 100 |
| KIDNE2003357 | 0 | 0 | 0 | 0 | 0 | 0 | 51.547 |
| KIDNE2003373 | 0 | 0 | 0 | 0 | 0 | 0 | 54.019 |
| KIDNE2003377 | 0 | 0 | 0 | 0 | 0 | 0 | 100 |
| KIDNE2003507 | 0 | 0 | 0 | 0 | 0 | 0 | 23.607 |
| KIDNE2003752 | 0 | 0 | 0 | 0 | 0 | 0 | 100 |
| KIDNE2003764 | 0 | 0 | 39.509 | 0 | 0 | 0 | 37.554 |
| KIDNE2003837 | 0 | 0 | 0 | 0 | 0 | 0 | 100 |
| KIDNE2003941 | 0 | 0 | 0 | 0 | 0 | 0 | 1.758 |
| KIDNE2003994 | 0 | 0 | 0 | 0 | 0 | 0 | 28.111 |
| KIDNE2004034 | 0 | 0 | 0 | 0 | 0 | 0 | 100 |
| KIDNE2004054 | 0 | 0 | 0 | 0 | 0 | 0 | 45.509 |
| KIDNE2004084 | 0 | 0 | 0 | 0 | 0 | 0 | 100 |
| KIDNE2004095 | 0 | 0 | 0 | 0 | 0 | 0 | 100 |
| KIDNE2004115 | 0 | 0 | 0 | 0 | 0 | 0 | 83.085 |
| KIDNE2004233 | 0 | 0 | 0 | 0 | 0 | 0 | 100 |
| KIDNE2004262 | 0 | 0 | 0 | 0 | 0 | 0 | 100 |
| KIDNE2004279 | 0 | 0 | 0 | 0 | 0 | 0 | 100 |
| KIDNE2004294 | 0 | 0 | 0 | 0 | 0 | 0 | 100 |
| KIDNE2004295 | 0 | 0 | 23.416 | 0 | 0 | 0 | 22.257 |
| KIDNE2004305 | 0 | 0 | 0 | 0 | 0 | 0 | 65.363 |
| KIDNE2004344 | 0 | 0 | 0 | 0 | 0 | 0 | 80.699 |
| KIDNE2004394 | 0 | 0 | 0 | 0 | 0 | 0 | 100 |
| KIDNE2004411 | 0 | 0 | 0 | 0 | 0 | 0 | 59.417 |
| KIDNE2004475 | 0 | 0 | 0 | 0 | 9.083 | 0 | 8.974 |
| KIDNE2004519 | 0 | 0 | 0 | 0 | 0 | 0 | 100 |
| KIDNE2004520 | 0 | 0 | 0 | 0 | 0 | 0 | 100 |
| KIDNE2004531 | 0 | 0 | 0 | 0 | 0 | 0 | 80.699 |
| KIDNE2004534 | 0 | 0 | 0 | 0 | 13.171 | 0 | 13.013 |
| KIDNE2004562 | 0 | 0 | 0 | 0 | 0 | 0 | 100 |
| KIDNE2004579 | 0 | 0 | 0 | 0 | 0 | 0 | 100 |
| KIDNE2004626 | 0 | 0 | 0 | 0 | 0 | 0 | 44.933 |
| KIDNE2004681 | 0 | 0 | 0 | 0 | 0 | 0 | 100 |
| KIDNE2004828 | 0 | 0 | 0 | 0 | 0 | 0 | 100 |
| KIDNE2004846 | 0 | 0 | 0 | 0 | 0 | 0 | 22.995 |
| KIDNE2004864 | 0 | 0 | 0 | 0 | 0 | 0 | 34.038 |
| KIDNE2004879 | 0 | 0 | 0 | 0 | 0 | 0 | 100 |
| KIDNE2004981 | 0 | 0 | 0 | 0 | 0 | 0 | 100 |
| KIDNE2004985 | 0 | 0 | 0 | 0 | 0 | 0 | 100 |
| KIDNE2004999 | 0 | 0 | 0 | 0 | 0 | 0 | 100 |
| KIDNE2005038 | 0 | 0 | 0 | 0 | 0 | 0 | 100 |
| KIDNE2005042 | 0 | 0 | 0 | 0 | 0 | 0 | 28.069 |
| KIDNE2005062 | 0 | 0 | 0 | 0 | 0 | 0 | 100 |
| KIDNE2005266 | 0 | 0 | 0 | 0 | 0 | 0 | 100 |
| KIDNE2005296 | 0 | 0 | 0 | 0 | 0 | 0 | 100 |
| KIDNE2005321 | 0 | 0 | 0 | 0 | 0 | 0 | 34.448 |
| KIDNE2005327 | 0 | 0 | 0 | 0 | 0 | 0 | 100 |
| KIDNE2005336 | 0 | 0 | 0 | 0 | 0 | 0 | 58.307 |
| KIDNE2005400 | 0 | 0 | 0 | 0 | 0 | 0 | 100 |
| KIDNE2005424 | 0 | 0 | 0 | 0 | 0 | 0 | 100 |
| KIDNE2005477 | 0 | 0 | 0 | 0 | 0 | 0 | 100 |
| KIDNE2005526 | 0 | 0 | 0 | 0 | 0 | 0 | 100 |
| KIDNE2005543 | 0 | 0 | 0 | 0 | 0 | 0 | 62.084 |
| KIDNE2005603 | 0 | 0 | 0 | 0 | 0 | 0 | 100 |
| KIDNE2005629 | 0 | 0 | 0 | 0 | 0 | 0 | 100 |
| KIDNE2005676 | 0 | 0 | 0 | 0 | 0 | 0 | 4.905 |
| KIDNE2005781 | 0 | 0 | 0 | 0 | 0 | 0 | 100 |
| KIDNE2005798 | 0 | 0 | 0 | 0 | 0 | 0 | 100 |
| KIDNE2005854 | 0 | 0 | 0 | 0 | 0 | 0 | 100 |
| KIDNE2005908 | 0 | 0 | 0 | 0 | 0 | 0 | 100 |
| KIDNE2005937 | 0 | 0 | 0 | 0 | 0 | 0 | 94.471 |
| KIDNE2006009 | 0 | 25.959 | 0 | 0 | 0 | 0 | 12.853 |
| KIDNE2006014 | 0 | 0 | 0 | 0 | 0 | 0 | 10.352 |
| KIDNE2006030 | 0 | 0 | 0 | 0 | 0 | 0 | 7.440 |
| KIDNE2006039 | 0 | 0 | 0 | 0 | 0 | 0 | 55.033 |
| KIDNE2006053 | 0 | 0 | 0 | 0 | 0 | 0 | 100 |
| KIDNE2006062 | 0 | 0 | 0 | 0 | 0 | 0 | 39.197 |
| KIDNE2006092 | 0 | 0 | 0 | 0 | 0 | 0 | 100 |
| KIDNE2006145 | 0 | 0 | 0 | 0 | 0 | 0 | 100 |
| KIDNE2006149 | 0 | 0 | 0 | 0 | 0 | 0 | 100 |
| KIDNE2006210 | 0 | 0 | 0 | 0 | 0 | 0 | 48.873 |
| KIDNE2006248 | 27.288 | 0 | 1.140 | 0 | 0 | 8.547 | 1.084 |
| KIDNE2006299 | 0 | 0 | 0 | 0 | 0 | 0 | 100 |
| KIDNE2006334 | 0 | 0 | 0 | 0 | 0 | 0 | 100 |
| KIDNE2006353 | 0 | 0 | 14.344 | 0 | 0 | 0 | 13.634 |
| KIDNE2006376 | 0 | 0 | 0 | 0 | 0 | 0 | 100 |
| KIDNE2006378 | 0 | 0 | 0 | 0 | 0 | 0 | 100 |
| KIDNE2006465 | 0 | 0 | 0 | 0 | 0 | 0 | 10.787 |
| KIDNE2006580 | 0 | 0 | 0 | 0 | 0 | 0 | 48.900 |
| KIDNE2006652 | 0 | 0 | 0 | 0 | 0 | 0 | 90.326 |
| KIDNE2006687 | 0 | 0 | 0 | 0 | 0 | 0 | 100 |
| KIDNE2006733 | 0 | 0 | 0 | 0 | 0 | 0 | 100 |
| KIDNE2006760 | 0 | 0 | 24.742 | 0 | 0 | 0 | 23.517 |
| KIDNE2006775 | 0 | 0 | 0 | 0 | 0 | 0 | 6.387 |
| KIDNE2006811 | 0 | 0 | 11.935 | 0 | 0 | 0 | 11.345 |
| KIDNE2006820 | 0 | 0 | 0 | 0 | 0 | 0 | 100 |
| KIDNE2006880 | 0 | 0 | 11.775 | 0 | 0 | 0 | 11.193 |
| KIDNE2007005 | 0 | 0 | 0 | 0 | 0 | 0 | 66.711 |
| KIDNE2007040 | 0 | 0 | 0 | 0 | 0 | 0 | 74.510 |
| KIDNE2007077 | 0 | 0 | 0 | 0 | 0 | 0 | 100 |
| KIDNE2007186 | 0 | 0 | 0 | 0 | 0 | 0 | 66.711 |
| KIDNE2007222 | 0 | 0 | 0 | 0 | 0 | 0 | 100 |
| KIDNE2007328 | 0 | 0 | 3.245 | 0 | 0 | 0 | 3.084 |
| KIDNE2007352 | 0 | 0 | 3.049 | 3.107 | 0 | 11.428 | 5.797 |
| KIDNE2007356 | 0 | 0 | 0 | 0 | 0 | 0 | 49.573 |
| KIDNE2007422 | 0 | 0 | 0 | 0 | 0 | 0 | 100 |
| KIDNE2007569 | 0 | 0 | 0 | 0 | 0 | 0 | 100 |
| KIDNE2007810 | 0 | 0 | 0 | 0 | 0 | 0 | 100 |
| KIDNE2007811 | 0 | 0 | 0 | 0 | 0 | 0 | 29.868 |
| KIDNE2007910 | 0 | 0 | 0 | 0 | 0 | 0 | 31.595 |
| KIDNE2007944 | 0 | 0 | 0 | 0 | 0 | 0 | 35.045 |
| KIDNE2007954 | 0 | 0 | 7.732 | 0 | 0 | 0 | 7.349 |
| KIDNE2008022 | 0 | 0 | 0 | 0 | 0 | 0 | 84.210 |
| KIDNE2008048 | 0 | 3.777 | 0 | 0 | 0 | 0 | 3.740 |
| KIDNE2008069 | 0 | 0 | 0 | 0 | 0 | 0 | 4.069 |
| KIDNE2008072 | 0 | 0 | 0 | 0 | 0 | 0 | 100 |
| KIDNE2008116 | 0 | 0 | 0 | 0 | 0 | 0 | 4.045 |
| KIDNE2008117 | 0 | 0 | 0 | 0 | 0 | 0 | 100 |
| KIDNE2008218 | 0 | 0 | 0 | 0 | 0 | 0 | 100 |
| KIDNE2008315 | 0 | 0 | 0 | 0 | 0 | 0 | 100 |
| KIDNE2008362 | 0 | 0 | 0 | 0 | 16.112 | 0 | 15.919 |
| KIDNE2008378 | 0 | 0 | 0 | 0 | 0 | 0 | 100 |
| KIDNE2008403 | 0 | 0 | 0 | 0 | 0 | 0 | 4.741 |
| KIDNE2008404 | 0 | 0 | 0 | 0 | 0 | 0 | 32.332 |
| KIDNE2008446 | 0 | 0 | 0 | 0 | 0 | 0 | 100 |
| KIDNE2008473 | 0 | 0 | 0 | 0 | 0 | 0 | 100 |
| KIDNE2008480 | 0 | 0 | 0 | 0 | 0 | 0 | 100 |
| KIDNE2008649 | 0 | 0 | 0 | 0 | 0 | 0 | 89.133 |
| KIDNE2008666 | 0 | 0 | 0 | 0 | 0 | 0 | 77.710 |
| KIDNE2008697 | 0 | 0 | 0 | 0 | 0 | 0 | 100 |
| KIDNE2008758 | 0 | 0 | 0 | 0 | 0 | 0 | 75.867 |
| KIDNE2008788 | 0 | 0 | 0 | 0 | 0 | 0 | 100 |
| KIDNE2008795 | 0 | 0 | 0 | 0 | 0 | 0 | 100 |
| KIDNE2008824 | 0 | 0 | 0 | 0 | 0 | 0 | 100 |
| KIDNE2008832 | 0 | 0 | 0 | 0 | 0 | 0 | 100 |
| KIDNE2008869 | 0 | 0 | 0 | 0 | 0 | 0 | 100 |
| KIDNE2008914 | 0 | 0 | 0 | 0 | 0 | 0 | 60.644 |
| KIDNE2008975 | 0 | 0 | 0 | 0 | 0 | 0 | 100 |
| KIDNE2008987 | 0 | 0 | 0 | 0 | 0 | 0 | 10.043 |
| KIDNE2009019 | 0 | 0 | 0 | 0 | 0 | 0 | 73.728 |
| KIDNE2009109 | 0 | 0 | 0 | 0 | 0 | 0 | 100 |
| KIDNE2009180 | 0 | 0 | 0 | 0 | 0 | 0 | 100 |
| KIDNE2009191 | 0 | 0 | 0 | 0 | 0 | 0 | 100 |
| KIDNE2009332 | 0 | 0 | 0 | 0 | 0 | 0 | 65.735 |
| KIDNE2009367 | 0 | 0 | 0 | 0 | 2.659 | 0 | 2.628 |
| KIDNE2009426 | 0 | 0 | 0 | 0 | 0 | 0 | 100 |
| KIDNE2009467 | 0 | 0 | 0 | 0 | 0 | 0 | 100 |
| KIDNE2009490 | 0 | 0 | 0 | 0 | 0 | 0 | 100 |
| KIDNE2009553 | 0 | 0 | 0 | 0 | 0 | 0 | 100 |
| KIDNE2009605 | 0 | 0 | 0 | 0 | 0 | 0 | 52.649 |
| KIDNE2009628 | 0 | 0 | 0 | 0 | 0 | 0 | 100 |
| KIDNE2009647 | 0 | 0 | 0 | 0 | 0 | 0 | 100 |
| KIDNE2009676 | 0 | 0 | 0 | 0 | 0 | 0 | 100 |
| KIDNE2009723 | 0 | 0 | 0 | 0 | 0 | 0 | 100 |
| KIDNE2010007 | 0 | 0 | 0 | 0 | 0 | 0 | 100 |
| KIDNE2010049 | 0 | 0 | 0 | 0 | 0 | 0 | 11.987 |
| KIDNE2010052 | 0 | 0 | 0 | 0 | 0 | 0 | 100 |
| KIDNE2010084 | 0 | 0 | 0 | 0 | 0 | 0 | 100 |
| KIDNE2010137 | 0 | 0 | 0 | 0 | 0 | 0 | 100 |
| KIDNE2010151 | 0 | 0 | 0 | 0 | 0 | 0 | 100 |
| KIDNE2010264 | 0 | 0 | 0 | 0 | 0 | 0 | 100 |
| KIDNE2010265 | 0 | 0 | 0 | 0 | 0 | 0 | 8.237 |
| KIDNE2010271 | 0 | 0 | 0 | 0 | 0 | 0 | 100 |
| KIDNE2010419 | 0 | 0 | 0 | 0 | 0 | 0 | 100 |
| KIDNE2010430 | 0 | 0 | 0 | 0 | 0 | 0 | 33.246 |
| KIDNE2010574 | 0 | 0 | 0 | 0 | 0 | 0 | 100 |
| KIDNE2010674 | 0 | 0 | 0 | 0 | 0 | 0 | 100 |
| KIDNE2010739 | 0 | 0 | 0 | 0 | 0 | 0 | 100 |
| KIDNE2010750 | 0 | 0 | 0 | 0 | 0 | 0 | 74.264 |
| KIDNE2010762 | 0 | 0 | 0 | 0 | 0 | 0 | 100 |
| KIDNE2010863 | 0 | 0 | 0 | 0 | 0 | 0 | 100 |
| KIDNE2010973 | 0 | 0 | 0 | 0 | 0 | 0 | 100 |
| KIDNE2010989 | 0 | 0 | 0 | 0 | 0 | 0 | 100 |
| KIDNE2011200 | 0 | 0 | 0 | 0 | 0 | 0 | 100 |
| KIDNE2011314 | 0 | 0 | 0 | 0 | 0 | 0 | 75.867 |
| KIDNE2011508 | 0 | 0 | 0 | 0 | 0 | 0 | 65.178 |
| KIDNE2011532 | 0 | 0 | 0 | 0 | 0 | 0 | 100 |
| KIDNE2011635 | 0 | 0 | 0 | 0 | 0 | 0 | 48.502 |
| KIDNE2011752 | 0 | 0 | 0 | 0 | 0 | 0 | 83.085 |
| KIDNE2011782 | 0 | 0 | 0 | 0 | 0 | 0 | 100 |
| KIDNE2011858 | 0 | 0 | 0 | 0 | 0 | 0 | 100 |
| KIDNE2012009 | 0 | 0 | 0 | 0 | 0 | 0 | 33.186 |
| KIDNE2012025 | 0 | 0 | 0 | 0 | 0 | 0 | 16.362 |
| KIDNE2012188 | 0 | 0 | 0 | 0 | 0 | 0 | 62.256 |
| KIDNE2012291 | 0 | 0 | 0 | 0 | 0 | 0 | 83.085 |
| KIDNE2012316 | 0 | 0 | 0 | 0 | 0 | 0 | 59.126 |
| KIDNE2012361 | 0 | 0 | 0 | 0 | 0 | 0 | 100 |
| KIDNE2012440 | 0 | 0 | 0 | 13.230 | 0 | 0 | 6.171 |
| KIDNE2012453 | 0 | 0 | 0 | 0 | 0 | 0 | 100 |
| KIDNE2012563 | 0 | 0 | 0 | 0 | 0 | 0 | 100 |
| KIDNE2012601 | 0 | 0 | 0 | 0 | 0 | 0 | 100 |
| KIDNE2012613 | 0 | 0 | 0 | 0 | 0 | 0 | 100 |
| KIDNE2012710 | 0 | 0 | 0 | 0 | 0 | 0 | 33.012 |
| KIDNE2012745 | 0 | 0 | 0 | 0 | 0 | 0 | 100 |
| KIDNE2012775 | 0 | 0 | 0 | 0 | 0 | 0 | 100 |
| KIDNE2012784 | 0 | 0 | 0 | 0 | 0 | 0 | 59.375 |
| KIDNE2012945 | 0 | 0 | 51.268 | 0 | 0 | 0 | 48.732 |
| KIDNE2013045 | 0 | 0 | 0 | 0 | 0 | 0 | 100 |
| KIDNE2013095 | 0 | 0 | 0 | 0 | 0 | 0 | 100 |
| KIDNE2013158 | 0 | 0 | 0 | 0 | 0 | 0 | 63.240 |
| KIDNE2013218 | 0 | 0 | 0 | 0 | 0 | 0 | 100 |
| KIDNE2013263 | 0 | 0 | 0 | 0 | 0 | 0 | 100 |
| KIDNE2013346 | 0 | 0 | 0 | 0 | 0 | 0 | 72.727 |
| KIDNE2013388 | 0 | 0 | 0 | 0 | 0 | 0 | 100 |
| KIDNE2013413 | 0 | 0 | 0 | 0 | 0 | 0 | 65.363 |
| KIDNE2013489 | 0 | 0 | 0 | 0 | 0 | 0 | 100 |
| KIDNE2013639 | 0 | 0 | 0 | 0 | 0 | 0 | 100 |
| KIDNE2013731 | 0 | 0 | 0 | 0 | 0 | 0 | 100 |
| KIDNE2013734 | 0 | 0 | 0 | 0 | 0 | 0 | 100 |
| KIDNE2013775 | 0 | 0 | 0 | 0 | 0 | 0 | 100 |
| KIDNE2013801 | 0 | 0 | 0 | 0 | 0 | 0 | 100 |
| KIDNE2013845 | 0 | 0 | 4.099 | 0 | 0 | 0 | 3.897 |
| KIDNE2013866 | 0 | 0 | 0 | 0 | 0 | 0 | 84.210 |
| KIDNE2014087 | 0 | 0 | 0 | 0 | 0 | 0 | 100 |
| KIDNE2014112 | 0 | 0 | 0 | 0 | 0 | 0 | 100 |
| KIDNE2014119 | 0 | 0 | 0 | 0 | 0 | 0 | 49.697 |
| KIDNE2014170 | 0 | 0 | 0 | 0 | 0 | 0 | 100 |
| KIDNE2014184 | 0 | 0 | 0 | 0 | 0 | 0 | 100 |
| KIDNE2014268 | 0 | 0 | 0 | 0 | 0 | 0 | 100 |
| KIDNE2014290 | 0 | 0 | 10.285 | 5.240 | 0 | 0 | 2.444 |
| KIDNE2014298 | 0 | 0 | 0 | 0 | 0 | 0 | 100 |
| KIDNE2014320 | 0 | 0 | 0 | 0 | 0 | 0 | 75.867 |
| KIDNE2014325 | 0 | 26.636 | 13.875 | 0 | 0 | 0 | 13.188 |
| KIDNE2014415 | 0 | 0 | 0 | 0 | 0 | 0 | 100 |
| KIDNE2014489 | 0 | 0 | 0 | 0 | 0 | 0 | 100 |
| KIDNE2014496 | 0 | 0 | 0 | 0 | 0 | 0 | 100 |
| KIDNE2014661 | 0 | 0 | 0 | 0 | 0 | 0 | 100 |
| KIDNE2014717 | 0 | 0 | 0 | 0 | 0 | 0 | 100 |
| KIDNE2014789 | 0 | 0 | 0 | 0 | 0 | 0 | 100 |
| KIDNE2014808 | 0 | 0 | 0 | 0 | 0 | 0 | 75.683 |
| KIDNE2014890 | 0 | 0 | 0 | 0 | 0 | 0 | 100 |
| KIDNE2014978 | 0 | 0 | 0 | 0 | 0 | 0 | 77.579 |
| KIDNE2015073 | 0 | 0 | 0 | 0 | 0 | 0 | 100 |
| KIDNE2015221 | 0 | 0 | 0 | 0 | 0 | 0 | 100 |
| KIDNE2015244 | 0 | 0 | 0 | 0 | 0 | 0 | 100 |
| KIDNE2015313 | 0 | 0 | 0 | 0 | 0 | 0 | 100 |
| KIDNE2015433 | 0 | 0 | 0 | 0 | 0 | 0 | 30.805 |
| KIDNE2015445 | 0 | 0 | 0 | 0 | 0 | 0 | 11.977 |
| KIDNE2015483 | 0 | 0 | 0 | 0 | 0 | 0 | 100 |
| KIDNE2015515 | 0 | 0 | 0 | 0 | 0 | 0 | 100 |
| KIDNE2015546 | 0 | 0 | 0 | 0 | 0 | 0 | 48.502 |
| KIDNE2015556 | 0 | 0 | 0 | 0 | 0 | 0 | 100 |
| KIDNE2015598 | 0 | 0 | 0 | 0 | 0 | 0 | 80.699 |
| KIDNE2015660 | 0 | 0 | 0 | 0 | 0 | 0 | 100 |
| KIDNE2015710 | 0 | 0 | 0 | 0 | 0 | 0 | 6.526 |
| KIDNE2015987 | 0 | 0 | 0 | 0 | 0 | 0 | 100 |
| KIDNE2016000 | 0 | 0 | 0 | 0 | 0 | 0 | 100 |
| KIDNE2016036 | 0 | 0 | 0 | 0 | 0 | 0 | 100 |
| KIDNE2016096 | 0 | 0 | 0 | 0 | 0 | 0 | 100 |
| KIDNE2016101 | 0 | 0 | 0 | 0 | 0 | 0 | 83.085 |
| KIDNE2016142 | 0 | 0 | 0 | 0 | 0 | 0 | 100 |
| KIDNE2016188 | 0 | 0 | 0 | 0 | 0 | 0 | 100 |
| KIDNE2016327 | 0 | 0 | 0 | 0 | 0 | 0 | 60.998 |
| KIDNE2016371 | 0 | 0 | 0 | 0 | 0 | 0 | 31.410 |
| KIDNE2016388 | 0 | 0 | 0 | 0 | 0 | 0 | 100 |
| KIDNE2016464 | 0 | 0 | 0 | 0 | 0 | 0 | 100 |
| KIDNE2016539 | 0 | 0 | 0 | 0 | 50.301 | 0 | 49.699 |
| KIDNE2016570 | 0 | 0 | 0 | 0 | 0 | 0 | 84.210 |
| KIDNE2016918 | 0 | 0 | 0 | 0 | 0 | 0 | 100 |
| KIDNE2017007 | 0 | 0 | 0 | 0 | 0 | 0 | 100 |
| KIDNE2017040 | 0 | 0 | 0 | 0 | 0 | 0 | 11.723 |
| KIDNE2017052 | 0 | 0 | 0 | 0 | 0 | 0 | 80.699 |
| KIDNE2017151 | 0 | 0 | 0 | 0 | 0 | 0 | 100 |
| KIDNE2017153 | 0 | 0 | 0 | 0 | 0 | 0 | 83.085 |
| KIDNE2017315 | 0 | 0 | 0 | 0 | 0 | 0 | 100 |
| KIDNE2017332 | 0 | 0 | 0 | 0 | 0 | 0 | 100 |
| KIDNE2017343 | 0 | 0 | 0 | 0 | 0 | 0 | 46.057 |
| KIDNE2017454 | 0 | 0 | 0 | 0 | 0 | 0 | 100 |
| KIDNE2017603 | 0 | 0 | 0 | 0 | 0 | 0 | 100 |
| KIDNE2017956 | 0 | 0 | 0 | 0 | 0 | 0 | 100 |
| KIDNE2018071 | 0 | 0 | 0 | 0 | 0 | 0 | 49.105 |
| KIDNE2018166 | 0 | 0 | 0 | 0 | 0 | 0 | 100 |
| KIDNE2018167 | 0 | 0 | 0 | 0 | 0 | 0 | 100 |
| KIDNE2018217 | 0 | 0 | 0 | 0 | 0 | 0 | 21.156 |
| KIDNE2018254 | 0 | 0 | 0 | 0 | 0 | 0 | 100 |
| KIDNE2018268 | 0 | 0 | 0 | 0 | 0 | 0 | 100 |
| KIDNE2018269 | 0 | 0 | 0 | 0 | 0 | 0 | 100 |
| KIDNE2018287 | 0 | 0 | 0 | 0 | 0 | 0 | 100 |
| KIDNE2018294 | 0 | 0 | 0 | 0 | 0 | 0 | 100 |
| KIDNE2018352 | 0 | 0 | 13.079 | 0 | 0 | 0 | 12.432 |
| KIDNE2018462 | 0 | 0 | 0 | 0 | 0 | 0 | 100 |
| KIDNE2018493 | 0 | 0 | 0 | 0 | 0 | 0 | 12.423 |
| KIDNE2018617 | 0 | 0 | 0 | 0 | 0 | 0 | 100 |
| KIDNE2018678 | 0 | 0 | 0 | 0 | 0 | 0 | 100 |
| KIDNE2018727 | 0 | 0 | 0 | 0 | 0 | 0 | 100 |
| KIDNE2018863 | 0 | 0 | 0 | 0 | 0 | 0 | 100 |
| KIDNE2018891 | 0 | 0 | 0 | 0 | 0 | 0 | 21.260 |
| KIDNE2018989 | 0 | 0 | 0 | 0 | 0 | 0 | 100 |
| KIDNE2018996 | 0 | 0 | 0 | 0 | 0 | 0 | 100 |
| KIDNE2019074 | 0 | 0 | 0 | 0 | 0 | 0 | 100 |
| KIDNE2019187 | 0 | 0 | 0 | 0 | 0 | 0 | 100 |
| LIVER1000079 | 0 | 0 | 0 | 0 | 0 | 0 | 6.109 |
| LIVER1000104 | 0 | 0 | 0 | 0 | 0 | 0 | 43.110 |
| LIVER1000132 | 0 | 0 | 12.847 | 0 | 0 | 0 | 0 |
| LIVER2002842 | 0 | 0 | 14.004 | 0 | 0 | 0 | 0 |
| LIVER2003511 | 0 | 0 | 0 | 0 | 0 | 0 | 28.683 |
| LIVER2006006 | 0 | 5.007 | 0 | 0 | 0 | 0 | 0 |
| MAMGL1000032 | 0 | 1.135 | 0 | 0 | 0.569 | 0 | 1.685 |
| MAMGL1000035 | 0 | 0 | 0 | 0 | 0 | 0 | 0.702 |
| MAMGL1000173 | 0 | 1.866 | 0 | 0 | 0 | 0 | 0 |
| MESAN2000264 | 0 | 13.875 | 0 | 14.729 | 6.953 | 0 | 0 |
| MESAN2000267 | 0 | 5.767 | 1.502 | 0 | 4.335 | 0 | 0 |
| MESAN2000337 | 0 | 55.830 | 0 | 0 | 0 | 0 | 0 |
| MESAN2000572 | 0 | 0 | 0 | 0 | 0 | 0 | 3.564 |
| MESAN2000608 | 0 | 0 | 5.768 | 0 | 0 | 0 | 0 |
| MESAN2001980 | 0 | 0 | 49.727 | 0 | 0 | 0 | 0 |
| MESAN2002086 | 0 | 0 | 1.527 | 18.668 | 0 | 0 | 0 |
| MESAN2002519 | 0 | 0 | 0 | 0 | 0 | 0 | 16.169 |
| MESAN2002724 | 0 | 0 | 0 | 0 | 0 | 25.351 | 0 |
| MESAN2003622 | 0 | 0 | 2.204 | 8.983 | 2.121 | 0 | 0 |
| MESAN2005284 | 0 | 0 | 0 | 36.243 | 17.110 | 0 | 0 |
| MESAN2005689 | 0 | 0 | 0 | 0 | 0 | 0 | 10.816 |
| MESAN2005766 | 0 | 0 | 0 | 0 | 3.480 | 27.109 | 0 |
| MESAN2006563 | 0 | 4.862 | 0 | 0 | 0 | 0 | 0 |
| MESAN2006971 | 0 | 42.308 | 0 | 0 | 0 | 0 | 0 |
| MESAN2008679 | 0 | 0 | 0 | 0 | 24.725 | 0 | 0 |
| MESAN2013845 | 0 | 39.288 | 0 | 0 | 0 | 0 | 0 |
| MESAN2015708 | 0 | 65.504 | 0 | 0 | 0 | 0 | 0 |
| NHNPC2000062 | 0 | 0 | 0 | 0 | 0 | 0 | 7.306 |
| NHNPC2000187 | 0 | 0 | 1.660 | 6.765 | 0 | 0 | 0 |
| NHNPC2000206 | 0 | 0 | 0 | 5.133 | 0 | 0 | 0 |
| NHNPC2001931 | 0 | 0 | 0 | 3.567 | 0 | 0 | 0 |
| NOVAR1000091 | 0 | 0 | 0 | 0 | 6.833 | 0 | 0 |
| NOVAR2000038 | 0 | 0 | 2.278 | 0 | 0 | 0 | 2.166 |
| NT2NE1000004 | 0 | 0 | 30.290 | 0 | 0 | 0 | 0 |
| NT2NE1000163 | 0 | 2.852 | 0 | 0 | 0 | 0 | 0 |
| NT2NE2000174 | 0 | 2.687 | 0 | 2.852 | 4.039 | 0 | 0 |
| NT2NE2000195 | 0 | 0 | 0 | 0 | 5.424 | 0 | 0 |
| NT2NE2000260 | 0 | 0 | 0.443 | 0 | 0 | 3.321 | 0 |
| NT2NE2000575 | 0 | 0 | 2.645 | 0 | 1.697 | 0 | 3.352 |
| NT2NE2000707 | 0 | 0 | 2.455 | 5.002 | 0 | 0 | 3.500 |
| NT2NE2000763 | 0 | 0 | 0 | 8.970 | 0 | 0 | 0 |
| NT2NE2000963 | 0 | 0 | 1.683 | 0 | 0 | 0 | 0.800 |
| NT2NE2001000 | 0 | 0 | 3.156 | 0 | 0 | 0 | 0 |
| NT2NE2001048 | 0 | 10.539 | 0 | 0 | 0 | 0 | 10.436 |
| NT2NE2001176 | 0 | 0 | 0 | 0 | 4.030 | 0 | 0 |
| NT2NE2001435 | 0 | 0 | 7.799 | 0 | 0 | 0 | 0 |
| NT2NE2001530 | 0 | 49.265 | 0 | 0 | 0 | 0 | 0 |
| NT2NE2001655 | 0 | 0 | 0 | 0 | 0 | 0 | 4.881 |
| NT2NE2002870 | 0 | 16.073 | 0 | 0 | 0 | 0 | 0 |
| NT2NE2003393 | 0 | 9.051 | 0 | 0 | 0 | 0 | 0 |
| NT2NE2004519 | 0 | 0 | 0 | 13.393 | 0 | 0 | 0 |
| NT2NE2004716 | 0 | 0 | 7.040 | 0 | 0 | 0 | 0 |
| NT2NE2006103 | 0 | 0 | 31.757 | 0 | 0 | 0 | 0 |
| NT2NE2010842 | 0 | 0 | 0 | 0 | 3.327 | 0 | 0 |
| NT2NE2012790 | 0 | 0 | 0 | 0 | 21.167 | 0 | 10 |
| NT2NE2014104 | 0 | 0 | 0 | 12.545 | 0 | 0 | 0 |
| NT2NE2018180 | 0 | 0 | 0 | 0 | 13.268 | 0 | 0 |
| NT2NE2019076 | 0 | 0 | 0 | 67.346 | 0 | 0 | 0 |
| NT2RI1000027 | 0 | 0 | 3.708 | 0 | 7.135 | 0 | 0 |
| NT2RI1000127 | 0 | 0 | 0 | 0 | 6.404 | 0 | 0 |
| NT2RI2000064 | 0 | 3.790 | 0 | 0 | 3.798 | 0 | 0 |
| NT2RI2000107 | 0 | 0 | 8.496 | 0 | 0 | 0 | 0 |
| NT2RI2000263 | 0 | 0 | 6.274 | 0 | 6.036 | 0 | 0 |
| NT2RI2000282 | 0 | 6.686 | 1.161 | 11.829 | 0 | 0 | 2.207 |
| NT2RI2000284 | 0 | 3.210 | 6.688 | 0 | 1.609 | 0 | 0 |
| NT2RI2000578 | 0 | 0 | 0 | 2.499 | 1.180 | 0 | 0 |
| NT2RI2000671 | 0 | 7.406 | 0 | 0 | 0 | 0 | 3.667 |
| NT2RI2000685 | 0 | 0 | 3.634 | 0 | 0 | 0 | 6.908 |
| NT2RI2000727 | 0 | 1.822 | 0 | 1.934 | 0 | 0 | 0.902 |
| NT2RI2000974 | 0 | 15.188 | 0 | 0 | 0 | 0 | 0 |
| NT2RI2001054 | 0 | 8.379 | 0 | 4.447 | 0 | 0 | 2.074 |
| NT2RI2001385 | 0 | 27.741 | 0 | 0 | 13.902 | 0 | 0 |
| NT2RI2001540 | 0 | 1.202 | 0.626 | 2.127 | 2.009 | 0 | 0.397 |
| NT2RI2001657 | 0 | 7.535 | 3.925 | 0 | 0 | 0 | 20.519 |
| NT2RI2001731 | 0 | 0 | 0 | 0 | 6.683 | 0 | 6.603 |
| NT2RI2001846 | 0 | 34.698 | 0 | 0 | 0 | 0 | 17.180 |
| NT2RI2001859 | 0 | 6.833 | 0 | 0 | 0 | 0 | 0 |
| NT2RI2001910 | 0 | 0 | 27.394 | 0 | 0 | 0 | 0 |
| NT2RI2002091 | 7.063 | 4.250 | 12.607 | 1.704 | 0 | 3.318 | 0.561 |
| NT2RI2002152 | 0 | 0 | 0 | 0 | 8.875 | 0 | 0 |
| NT2RI2002266 | 0 | 0 | 0 | 0 | 0 | 0 | 8.738 |
| NT2RI2002316 | 0 | 0 | 0 | 0 | 2.829 | 0 | 0 |
| NT2RI2002391 | 0 | 0 | 8.923 | 0 | 0 | 0 | 0 |
| NT2RI2002554 | 0 | 0 | 0 | 0 | 0 | 0 | 6.321 |
| NT2RI2002729 | 0 | 14.810 | 0 | 0 | 0 | 57.819 | 0 |
| NT2RI2003184 | 0 | 0 | 0 | 0 | 0 | 0 | 65.735 |
| NT2RI2003304 | 0 | 1.848 | 1.925 | 0 | 0 | 0 | 0.915 |
| NT2RI2003317 | 0 | 0 | 5.781 | 0 | 11.124 | 0 | 0 |
| NT2RI2003344 | 0 | 0 | 7.142 | 0 | 0 | 0 | 0 |
| NT2RI2003419 | 0 | 79.486 | 0 | 0 | 0 | 0 | 0 |
| NT2RI2003695 | 0 | 0 | 2.888 | 0 | 0 | 0 | 0 |
| NT2RI2003738 | 0 | 2.464 | 0.642 | 2.615 | 0.617 | 9.618 | 0 |
| NT2RI2003921 | 0 | 0 | 0 | 0 | 0 | 39.112 | 0 |
| NT2RI2003993 | 0 | 0 | 13.924 | 0 | 0 | 0 | 0 |
| NT2RI2004209 | 0 | 0 | 26.927 | 0 | 0 | 0 | 25.595 |
| NT2RI2004230 | 0 | 0 | 4.208 | 0 | 0 | 0 | 0 |
| NT2RI2004312 | 0 | 0 | 0 | 0 | 53.560 | 0 | 0 |
| NT2RI2004535 | 0 | 13.478 | 0 | 0 | 0 | 0 | 6.673 |
| NT2RI2004608 | 0 | 0 | 0 | 0 | 13.055 | 0 | 0 |
| NT2RI2004884 | 0 | 0 | 0 | 5.775 | 0 | 0 | 0 |
| NT2RI2005150 | 0 | 0 | 5.055 | 0 | 7.294 | 0 | 0 |
| NT2RI2005335 | 0 | 0 | 0 | 0 | 1.366 | 0 | 0 |
| NT2RI2005368 | 0 | 0 | 0 | 0 | 0 | 0 | 7.044 |
| NT2RI2005405 | 0 | 5.930 | 2.059 | 0 | 0 | 0 | 2.936 |
| NT2RI2005713 | 0 | 0 | 0 | 0 | 0 | 0 | 10.311 |
| NT2RI2005723 | 0 | 7.067 | 1.841 | 0 | 1.771 | 0 | 0 |
| NT2RI2005814 | 0 | 0 | 0 | 9.735 | 0 | 0 | 0 |
| NT2RI2005818 | 0 | 0 | 0 | 0 | 8.987 | 0 | 0 |
| NT2RI2006070 | 0 | 0 | 7.538 | 0 | 0 | 0 | 0 |
| NT2RI2006412 | 0 | 5.772 | 3.007 | 0 | 0 | 0 | 0 |
| NT2RI2006553 | 0 | 26.043 | 0 | 0 | 0 | 0 | 12.894 |
| NT2RI2006565 | 0 | 0 | 21.916 | 0 | 0 | 0 | 0 |
| NT2RI2006853 | 0 | 0 | 8.227 | 0 | 0 | 0 | 7.820 |
| NT2RI2006856 | 0 | 0 | 1.718 | 0 | 0 | 0 | 0 |
| NT2RI2007054 | 0 | 0 | 0 | 0 | 0 | 0 | 9.052 |
| NT2RI2007084 | 0 | 22.981 | 11.971 | 8.131 | 0 | 0 | 0 |
| NT2RI2007133 | 0 | 0 | 1.439 | 0 | 0 | 0 | 0 |
| NT2RI2007377 | 0 | 0 | 4.296 | 0 | 0 | 0 | 0 |
| NT2RI2007498 | 0 | 0 | 0 | 6.096 | 0 | 0 | 0 |
| NT2RI2007572 | 0 | 5.261 | 0 | 5.585 | 0 | 0 | 0 |
| NT2RI2007593 | 0 | 23.199 | 0 | 0 | 0 | 0 | 0 |
| NT2RI2007629 | 0 | 0 | 0 | 15.067 | 0 | 0 | 0 |
| NT2RI2007884 | 0 | 0 | 0 | 0 | 8.958 | 0 | 8.851 |
| NT2RI2007987 | 0 | 0 | 0 | 0 | 0 | 0 | 6.277 |
| NT2RI2008045 | 0 | 0 | 0 | 0 | 0 | 0 | 2.707 |
| NT2RI2008336 | 0 | 2.821 | 0 | 0 | 1.414 | 0 | 0 |
| NT2RI2008396 | 0 | 9.311 | 1.617 | 0 | 0 | 12.118 | 4.610 |
| NT2RI2008481 | 64.910 | 0 | 0 | 0 | 0 | 0 | 0 |
| NT2RI2008526 | 0 | 0 | 2.812 | 0 | 2.705 | 0 | 0 |
| NT2RI2008749 | 49.624 | 0 | 0 | 4.226 | 0 | 0 | 0 |
| NT2RI2009037 | 0 | 0 | 3.092 | 6.301 | 0 | 0 | 0 |
| NT2RI2009065 | 0 | 0 | 0 | 3.491 | 1.648 | 0 | 0 |
| NT2RI2009066 | 0 | 0 | 20.105 | 0 | 0 | 0 | 9.555 |
| NT2RI2009173 | 0 | 0 | 4.091 | 0 | 0 | 0 | 0 |
| NT2RI2009194 | 0 | 13.840 | 0 | 0 | 0 | 0 | 0 |
| NT2RI2009239 | 0 | 0 | 5.126 | 0 | 0 | 0 | 0 |
| NT2RI2009406 | 0 | 0 | 0 | 0 | 0 | 0 | 9.808 |
| NT2RI2009583 | 0 | 0 | 0 | 0 | 0 | 0 | 10.920 |
| NT2RI2012350 | 0 | 0 | 0 | 0 | 0 | 11.003 | 0 |
| NT2RI2015342 | 0 | 0 | 0 | 0 | 10.616 | 0 | 0 |
| NT2RI3001348 | 0 | 73.590 | 0 | 0 | 0 | 0 | 0 |
| NT2RI3007543 | 0 | 0 | 0 | 6.341 | 2.994 | 0 | 0 |
| NT2RP6000005 | 0 | 0 | 8.067 | 0 | 0 | 0 | 0 |
| NT2RP6000072 | 0 | 0 | 0 | 12.077 | 0 | 0 | 0 |
| NT2RP6000078 | 0 | 0 | 0 | 0 | 0 | 19.769 | 0 |
| NT2RP6000123 | 0 | 2.756 | 1.436 | 0 | 1.381 | 0 | 1.365 |
| NT2RP7000076 | 0 | 4.291 | 0 | 0 | 0 | 0 | 0 |
| NT2RP7000173 | 0 | 0 | 2.039 | 0 | 0 | 0 | 0 |
| NT2RP7000271 | 0 | 21.355 | 0 | 0 | 0 | 0 | 0 |
| NT2RP7000477 | 0 | 0 | 0 | 0 | 0 | 0 | 6.897 |
| NT2RP7000600 | 0 | 0 | 8.127 | 0 | 0 | 0 | 0 |
| NT2RP7000906 | 0 | 0 | 4.785 | 9.750 | 0 | 0 | 0 |
| NT2RP7001166 | 0 | 0 | 2.743 | 0 | 0 | 0 | 0 |
| NT2RP7001283 | 0 | 0 | 0 | 4.207 | 0 | 15.473 | 0 |
| NT2RP7001962 | 0 | 0 | 9.743 | 0 | 0 | 0 | 0 |
| NT2RP7002449 | 0 | 0 | 0 | 15.099 | 0 | 0 | 0 |
| NT2RP7002829 | 0 | 0 | 32.071 | 0 | 0 | 0 | 0 |
| NT2RP7003688 | 0 | 5.199 | 0 | 0 | 0 | 0 | 0 |
| NT2RP7004348 | 0 | 0 | 0 | 0 | 0 | 0 | 7.879 |
| NT2RP7004373 | 0 | 0 | 0 | 0 | 0 | 0 | 8.206 |
| NT2RP7004428 | 0 | 0 | 12.369 | 0 | 0 | 0 | 0 |
| NT2RP7004728 | 0 | 0 | 0 | 0 | 0 | 0 | 5.480 |
| NT2RP7004751 | 0 | 0 | 0 | 3.498 | 3.303 | 0 | 0 |
| NT2RP7004884 | 0 | 0 | 0 | 0 | 0 | 0 | 9.571 |
| NT2RP7004911 | 0 | 0 | 0 | 0 | 0 | 48.454 | 0 |
| NT2RP7004946 | 0 | 6.062 | 3.157 | 0 | 3.038 | 0 | 0 |
| NT2RP7006141 | 0 | 0 | 0 | 0 | 0 | 0 | 2.575 |
| NT2RP7006717 | 0 | 4.632 | 0 | 0 | 2.321 | 0 | 0 |
| NT2RP7006886 | 0 | 0 | 0 | 0 | 33.560 | 0 | 0 |
| NT2RP7006986 | 0 | 0 | 0 | 0 | 0 | 0 | 13.097 |
| NT2RP7007154 | 0 | 0 | 2.092 | 0 | 0 | 0 | 0 |
| NT2RP7007177 | 0 | 0 | 4.995 | 0 | 4.805 | 0 | 0 |
| NT2RP7007387 | 0 | 0 | 0 | 15.341 | 0 | 0 | 7.156 |
| NT2RP7007610 | 0 | 0 | 10.615 | 0 | 0 | 0 | 0 |
| NT2RP7008015 | 88.555 | 0 | 0 | 0 | 0 | 0 | 0 |
| NT2RP7008190 | 0 | 0 | 0 | 0 | 4.681 | 0 | 4.625 |
| NT2RP7014910 | 0 | 0 | 0 | 0 | 4.219 | 0 | 0 |
| NT2RP7018126 | 0 | 0 | 0 | 0 | 59.416 | 0 | 0 |
| NT2RP7020112 | 0 | 0 | 0 | 0 | 0 | 0 | 59.126 |
| NTONG1000098 | 0 | 0 | 0 | 0 | 0 | 0 | 2.221 |
| NTONG1000246 | 0 | 0 | 11.470 | 11.687 | 0 | 0 | 0 |
| NTONG1000257 | 0 | 7.749 | 0 | 0 | 0 | 0 | 0 |
| NTONG2000800 | 0 | 0 | 3.884 | 0 | 0 | 0 | 1.846 |
| NTONG2001137 | 0 | 0 | 4.198 | 0 | 4.038 | 0 | 0 |
| NTONG2001567 | 0 | 0 | 5.623 | 11.459 | 0 | 0 | 0 |
| NTONG2001612 | 0 | 0 | 4.117 | 0 | 7.922 | 0 | 0 |
| NTONG2001676 | 0 | 0 | 0 | 0 | 25.171 | 0 | 0 |
| NTONG2003839 | 0 | 0 | 0 | 19.596 | 0 | 0 | 0 |
| NTONG2004918 | 0 | 0 | 0 | 0 | 22.850 | 0 | 0 |
| NTONG2005520 | 0 | 4.687 | 0 | 0 | 0 | 0 | 2.321 |
| NTONG2007658 | 0 | 0 | 0 | 0 | 7.061 | 0 | 0 |
| NTONG2008483 | 0 | 0 | 2.930 | 0 | 2.819 | 0 | 0 |
| OCBBF1000086 | 0 | 0 | 0 | 0 | 0 | 0 | 2.669 |
| OCBBF1000114 | 0 | 0 | 0 | 0 | 8.995 | 0 | 0 |
| OCBBF1000188 | 0 | 0 | 0 | 0 | 3.674 | 0 | 0 |
| OCBBF2000467 | 0 | 14.202 | 0 | 0 | 0 | 0 | 0 |
| OCBBF2000522 | 0 | 17.805 | 0 | 0 | 0 | 0 | 0 |
| OCBBF2000824 | 0 | 0 | 0 | 44.912 | 0 | 0 | 0 |
| OCBBF2000846 | 0 | 0 | 0 | 5.470 | 0 | 0 | 0 |
| OCBBF2000986 | 0 | 0 | 0 | 0 | 3.400 | 26.492 | 0 |
| OCBBF2001494 | 0 | 0 | 0 | 0 | 0 | 0 | 73.728 |
| OCBBF2001586 | 0 | 0 | 0 | 0 | 12.652 | 0 | 0 |
| OCBBF2001687 | 0 | 0 | 0 | 0 | 0 | 0 | 6.706 |
| OCBBF2002083 | 0 | 0 | 40.304 | 0 | 0 | 0 | 0 |
| OCBBF2004757 | 0 | 0 | 0 | 0 | 8.745 | 0 | 0 |
| OCBBF2006148 | 0 | 0 | 24.838 | 0 | 0 | 0 | 0 |
| OCBBF2006172 | 0 | 0 | 0 | 0 | 18.501 | 0 | 0 |
| OCBBF2007829 | 0 | 0 | 0 | 4.104 | 1.937 | 0 | 0 |
| OCBBF2008041 | 0 | 14.718 | 0 | 0 | 0 | 0 | 0 |
| OCBBF2008569 | 0 | 0 | 0 | 0 | 73.961 | 0 | 0 |
| OCBBF2008760 | 0 | 0 | 0 | 0 | 58.681 | 0 | 0 |
| OCBBF2009772 | 0 | 8.291 | 0 | 0 | 0 | 0 | 0 |
| OCBBF2010830 | 0 | 0 | 3.227 | 6.577 | 3.105 | 0 | 0 |
| OCBBF2011160 | 0 | 0 | 0 | 0 | 0 | 0 | 60.858 |
| OCBBF2011536 | 0 | 0 | 7.317 | 0 | 0 | 0 | 0 |
| OCBBF2012553 | 0 | 8.258 | 4.302 | 0 | 0 | 0 | 0 |
| OCBBF2013843 | 0 | 0 | 0 | 3.994 | 0 | 0 | 0 |
| OCBBF2014064 | 0 | 0 | 0 | 0 | 0 | 37.548 | 0 |
| OCBBF2014292 | 0 | 0 | 0 | 0 | 3.655 | 0 | 3.611 |
| OCBBF2014541 | 0 | 0 | 28.761 | 0 | 0 | 0 | 0 |
| OCBBF2017035 | 0 | 0 | 0 | 0 | 0 | 0 | 4.226 |
| OCBBF2017893 | 0 | 0 | 6.362 | 0 | 0 | 0 | 0 |
| OCBBF2018707 | 0 | 0 | 0 | 0 | 7.936 | 0 | 0 |
| OCBBF2018956 | 0 | 11.917 | 0 | 0 | 0 | 0 | 0 |
| OCBBF2021020 | 0 | 0 | 0 | 0 | 0 | 73.294 | 0 |
| OCBBF2035226 | 0 | 0 | 0 | 0 | 2.073 | 0 | 0 |
| OCBBF3000323 | 0 | 0 | 12.198 | 0 | 0 | 0 | 0 |
| OCBBF3004487 | 0 | 0 | 0 | 0 | 15.987 | 0 | 0 |
| PEBLM1000174 | 0 | 0 | 0 | 0 | 0 | 0 | 16.672 |
| PEBLM2000147 | 0 | 0 | 0 | 0 | 0 | 35.070 | 0 |
| PEBLM2000222 | 0 | 0 | 0 | 0 | 2.050 | 0 | 0 |
| PEBLM2000267 | 0 | 0 | 0 | 0 | 14.884 | 0 | 0 |
| PEBLM2000308 | 0 | 0 | 7.271 | 0 | 0 | 0 | 0 |
| PEBLM2000395 | 0 | 6.409 | 0 | 0 | 6.423 | 0 | 0 |
| PEBLM2004666 | 0 | 0 | 0 | 0 | 0 | 0 | 12.953 |
| PEBLM2007774 | 0 | 0 | 1.105 | 0 | 0 | 0 | 1.050 |
| PERIC2002452 | 0 | 0 | 0 | 0 | 34.376 | 0 | 0 |
| PLACE5000172 | 0 | 0 | 0 | 0 | 0 | 0 | 13.594 |
| PLACE6000314 | 0 | 0 | 55.942 | 0 | 0 | 0 | 0 |
| PLACE6000379 | 0 | 0 | 1.943 | 1.320 | 0 | 0 | 1.847 |
| PLACE6000463 | 0 | 0 | 28.618 | 0 | 0 | 0 | 0 |
| PLACE6001443 | 0 | 0 | 0 | 0 | 0 | 0 | 6.848 |
| PLACE6001923 | 0 | 44.258 | 0 | 0 | 0 | 0 | 0 |
| PLACE6002084 | 0 | 2.600 | 0 | 0 | 0 | 0 | 2.575 |
| PLACE6002960 | 0 | 0 | 20.354 | 0 | 0 | 0 | 0 |
| PLACE6003038 | 0 | 25.411 | 0 | 0 | 0 | 0 | 0 |
| PLACE6003383 | 0 | 0 | 0 | 8.921 | 0 | 0 | 0 |
| PLACE6004312 | 0 | 0 | 2.212 | 0 | 0 | 0 | 2.103 |
| PLACE6004396 | 0 | 0 | 67.330 | 0 | 0 | 0 | 0 |
| PLACE6006549 | 0 | 0 | 11.715 | 0 | 0 | 0 | 0 |
| PLACE6007436 | 0 | 0 | 0 | 0 | 0 | 0 | 12.525 |
| PLACE6009560 | 0 | 0 | 0 | 80.768 | 0 | 0 | 0 |
| PLACE6010484 | 0 | 0 | 0 | 0 | 0 | 0 | 5.389 |
| PLACE6011057 | 0 | 0 | 0 | 0 | 0 | 0 | 7.857 |
| PLACE6011334 | 0 | 0 | 0 | 0 | 31.714 | 0 | 0 |
| PLACE6013222 | 0 | 0 | 18.072 | 0 | 0 | 0 | 0 |
| PLACE6016383 | 0 | 0 | 23.711 | 0 | 0 | 0 | 0 |
| PLACE6017626 | 0 | 0 | 0 | 0 | 0 | 0 | 49.481 |
| PLACE6018863 | 0 | 0 | 0 | 0 | 0 | 0 | 66.204 |
| PLACE7004103 | 0 | 9.106 | 0 | 0 | 0 | 0 | 0 |
| PROST1000033 | 0 | 0 | 40.409 | 0 | 0 | 0 | 0 |
| PROST1000097 | 0 | 0 | 0 | 0 | 0 | 0 | 2.050 |
| PROST1000120 | 0 | 0 | 12.095 | 0 | 0 | 0 | 0 |
| PROST1000144 | 0 | 22.079 | 0 | 11.718 | 0 | 0 | 0 |
| PROST1000226 | 0 | 0 | 6.562 | 0 | 0 | 0 | 0 |
| PROST1000343 | 0 | 0 | 0 | 0 | 0 | 0 | 9.903 |
| PROST2000036 | 0 | 0 | 0 | 0 | 0 | 77.280 | 0 |
| PROST2000138 | 0 | 2.441 | 1.907 | 3.239 | 0 | 0 | 2.115 |
| PROST2001180 | 0 | 0 | 0 | 0 | 8.761 | 0 | 0 |
| PROST2002651 | 0 | 0 | 21.937 | 0 | 0 | 0 | 0 |
| PROST2003338 | 0 | 0 | 0 | 8.366 | 0 | 0 | 7.804 |
| PROST2003517 | 0 | 0 | 0 | 0 | 18.579 | 0 | 0 |
| PROST2004095 | 0 | 0 | 0 | 3.498 | 0 | 0 | 0 |
| PROST2004270 | 0 | 4.140 | 2.157 | 8.790 | 0 | 0 | 0 |
| PROST2005285 | 0 | 0 | 27.376 | 0 | 0 | 0 | 0 |
| PROST2005604 | 0 | 0 | 0 | 23.249 | 0 | 0 | 0 |
| PROST2005793 | 0 | 0 | 14.383 | 0 | 0 | 0 | 0 |
| PROST2006260 | 0 | 0 | 0 | 0 | 14.368 | 0 | 0 |
| PROST2006536 | 0 | 0 | 0 | 0 | 7.830 | 0 | 0 |
| PROST2006737 | 0 | 17.066 | 0 | 0 | 0 | 0 | 0 |
| PROST2008271 | 0 | 0 | 14.374 | 0 | 0 | 0 | 0 |
| PROST2008489 | 0 | 0 | 0 | 19.362 | 0 | 0 | 0 |
| PROST2008770 | 0 | 0 | 0 | 5.830 | 0 | 0 | 0 |
| PROST2010782 | 0 | 0 | 0.892 | 0 | 0 | 0 | 1.696 |
| PROST2011577 | 0 | 0 | 0 | 0 | 3.631 | 0 | 0 |
| PROST2012007 | 0 | 0 | 0 | 8.483 | 4.005 | 31.201 | 0 |
| PROST2012353 | 0 | 0 | 0 | 0 | 0 | 0 | 32.771 |
| PROST2015287 | 0 | 0 | 0 | 0 | 37.880 | 0 | 0 |
| PROST2015464 | 0 | 0 | 0 | 0 | 0 | 0 | 41.857 |
| PROST2016195 | 0 | 0 | 0 | 0 | 0 | 0 | 20.387 |
| PROST2017203 | 0 | 0 | 4.763 | 0 | 0 | 0 | 0 |
| PROST2017529 | 0 | 0 | 0 | 18.971 | 0 | 0 | 0 |
| PROST2017692 | 0 | 0 | 0 | 16.523 | 2.600 | 0 | 0 |
| PROST2018583 | 0 | 0 | 33.898 | 0 | 0 | 0 | 0 |
| PUAEN1000065 | 0 | 0 | 2.457 | 0 | 0 | 0 | 4.670 |
| PUAEN1000164 | 34.934 | 0 | 0 | 0 | 2.809 | 0 | 0 |
| PUAEN1000239 | 0 | 0 | 0 | 0 | 10.939 | 0 | 0 |
| PUAEN2000374 | 28.090 | 2.254 | 0 | 2.392 | 0 | 8.798 | 0 |
| PUAEN2000497 | 0 | 18.152 | 0 | 0 | 0 | 0 | 0 |
| PUAEN2000535 | 70.586 | 0 | 0 | 0 | 0 | 0 | 0 |
| PUAEN2003018 | 0 | 0 | 12.294 | 0 | 0 | 0 | 46.744 |
| PUAEN2003954 | 0 | 0 | 0 | 0 | 38.474 | 0 | 0 |
| PUAEN2004400 | 0 | 0 | 0 | 0 | 25.421 | 0 | 0 |
| PUAEN2008939 | 0 | 0 | 12.588 | 0 | 0 | 0 | 0 |
| SALGL1000157 | 0 | 0 | 1.431 | 1.944 | 0 | 0 | 0 |
| SKMUS1000177 | 0 | 0 | 0 | 0 | 8.332 | 0 | 0 |
| SKMUS1000186 | 0 | 0 | 1.472 | 3.000 | 0 | 0 | 0 |
| SKMUS2000361 | 0 | 0 | 0 | 0 | 0 | 0 | 3.728 |
| SKMUS2000484 | 0 | 0 | 6.441 | 0 | 0 | 0 | 0 |
| SKMUS2000931 | 0 | 3.831 | 0 | 0 | 1.920 | 0 | 0 |
| SKMUS2001147 | 0 | 0 | 0 | 0 | 19.562 | 0 | 0 |
| SKMUS2001454 | 0 | 0 | 2.476 | 0 | 2.382 | 0 | 1.177 |
| SKMUS2001528 | 0 | 0 | 0 | 0 | 0 | 0 | 4.376 |
| SKMUS2001634 | 0 | 0 | 4.802 | 0 | 0 | 0 | 0 |
| SKMUS2002084 | 0 | 0 | 0 | 0 | 22.031 | 0 | 0 |
| SKNMC1000110 | 0 | 0 | 0 | 0 | 0 | 0 | 12.622 |
| SKNMC1000168 | 0 | 0 | 0 | 0 | 0 | 0 | 2.919 |
| SKNMC2000065 | 0 | 7.601 | 0 | 0 | 0 | 0 | 0 |
| SKNMC2000097 | 0 | 6.277 | 0 | 0 | 0 | 0 | 0 |
| SKNMC2000322 | 0 | 0 | 0 | 0 | 9.966 | 0 | 0 |
| SKNMC2000593 | 0 | 0 | 31.996 | 0 | 0 | 0 | 0 |
| SKNMC2000635 | 0 | 3.789 | 0.658 | 6.703 | 0 | 0 | 0 |
| SKNMC2000649 | 0 | 6.424 | 6.692 | 0 | 0 | 0 | 0 |
| SKNMC2000698 | 0 | 14.544 | 0 | 0 | 0 | 0 | 0 |
| SKNMC2001503 | 0 | 0 | 11.374 | 0 | 0 | 0 | 0 |
| SKNSH1000002 | 0 | 0 | 0 | 0 | 0 | 0 | 5.011 |
| SKNSH2000151 | 0 | 0 | 0 | 0 | 19.196 | 0 | 18.966 |
| SKNSH2000347 | 0 | 0 | 0 | 0 | 7.917 | 0 | 0 |
| SKNSH2000716 | 0 | 0 | 0 | 0 | 24.462 | 0 | 0 |
| SKNSH2000971 | 0 | 50.629 | 0 | 0 | 0 | 0 | 0 |
| SKNSH2006234 | 0 | 24.647 | 0 | 0 | 0 | 0 | 0 |
| SMINT1000016 | 0 | 0 | 0 | 0 | 0 | 0 | 25.659 |
| SMINT1000048 | 0 | 0 | 28.889 | 0 | 0 | 0 | 0 |
| SMINT1000131 | 0 | 0 | 3.359 | 13.690 | 0 | 0 | 0 |
| SMINT2000007 | 0 | 0 | 22.061 | 0 | 0 | 0 | 0 |
| SMINT2000018 | 0 | 0 | 15.442 | 0 | 0 | 0 | 0 |
| SMINT2000159 | 0 | 0 | 0 | 0 | 0 | 0 | 19.053 |
| SMINT2000239 | 0 | 0 | 30.764 | 0 | 0 | 0 | 0 |
| SMINT2000277 | 0 | 0 | 33.247 | 0 | 0 | 0 | 0 |
| SMINT2000396 | 0 | 0 | 4.490 | 0 | 0 | 0 | 0 |
| SMINT2000441 | 0 | 0 | 1.656 | 0 | 0 | 0 | 3.147 |
| SMINT2000602 | 0 | 0 | 2.016 | 0 | 0 | 15.111 | 0 |
| SMINT2000659 | 0 | 0 | 0 | 0 | 0 | 0 | 0.969 |
| SMINT2000933 | 0 | 0 | 3.738 | 0 | 0 | 0 | 3.553 |
| SMINT2001812 | 0 | 0 | 0 | 0 | 0 | 0 | 27.880 |
| SMINT2002314 | 0 | 0 | 0 | 0 | 0 | 33.271 | 0 |
| SMINT2002882 | 0 | 0 | 0 | 0 | 10.246 | 0 | 0 |
| SMINT2002884 | 0 | 0 | 13.548 | 0 | 0 | 0 | 0 |
| SMINT2003074 | 0 | 0 | 5.028 | 0 | 0 | 0 | 0 |
| SMINT2003169 | 0 | 3.312 | 1.725 | 0 | 0 | 12.930 | 0 |
| SMINT2003340 | 0 | 0 | 0 | 0 | 0 | 0 | 10.243 |
| SMINT2004023 | 0 | 0 | 5.594 | 0 | 0 | 0 | 0 |
| SMINT2004144 | 0 | 0 | 0 | 0 | 0 | 0 | 7.912 |
| SMINT2004280 | 0 | 0 | 0 | 0 | 0 | 0 | 44.418 |
| SMINT2005368 | 0 | 0 | 0 | 0 | 8.288 | 0 | 0 |
| SMINT2008960 | 0 | 0 | 0 | 0 | 0 | 0 | 4.770 |
| SMINT2010901 | 0 | 24.729 | 0 | 0 | 0 | 0 | 0 |
| SMINT2012122 | 0 | 0 | 12.822 | 0 | 0 | 0 | 0 |
| SPLEN1000106 | 0 | 0 | 0 | 0 | 0 | 0 | 8.115 |
| SPLEN1000144 | 0 | 0 | 38.347 | 0 | 0 | 0 | 0 |
| SPLEN1000165 | 0 | 0 | 0 | 0 | 0 | 0 | 17.398 |
| SPLEN1000166 | 0 | 0 | 8.292 | 0 | 0 | 0 | 0 |
| SPLEN2000047 | 0 | 0 | 0 | 0 | 0 | 0 | 57.532 |
| SPLEN2000134 | 0 | 0 | 0 | 0 | 0 | 0 | 73.057 |
| SPLEN2000222 | 0 | 0 | 44.799 | 0 | 0 | 0 | 0 |
| SPLEN2000255 | 0 | 0 | 5.633 | 0 | 0 | 0 | 0 |
| SPLEN2000258 | 0 | 0 | 0 | 32.721 | 0 | 0 | 0 |
| SPLEN2000348 | 0 | 0 | 10.260 | 0 | 0 | 0 | 0 |
| SPLEN2000505 | 0 | 0 | 0 | 3.150 | 0 | 0 | 0 |
| SPLEN2000874 | 0 | 0 | 1.788 | 0 | 0 | 0 | 0 |
| SPLEN2001157 | 0 | 0 | 0 | 0 | 9.705 | 0 | 0 |
| SPLEN2001245 | 98.056 | 0 | 0 | 0 | 0 | 0 | 0 |
| SPLEN2001761 | 0 | 0 | 0 | 0 | 0 | 0 | 37.096 |
| SPLEN2002467 | 0 | 0 | 0 | 24.349 | 0 | 0 | 0 |
| SPLEN2002744 | 0 | 0 | 0 | 16.670 | 0 | 0 | 0 |
| SPLEN2004181 | 0 | 0 | 28.873 | 0 | 0 | 0 | 0 |
| SPLEN2004220 | 0 | 0 | 0 | 0 | 0 | 79.064 | 0 |
| SPLEN2004880 | 0 | 0 | 1.080 | 0 | 0 | 0 | 0 |
| SPLEN2004984 | 0 | 0 | 9.634 | 0 | 0 | 0 | 0 |
| SPLEN2005429 | 0 | 0 | 26.875 | 0 | 0 | 0 | 0 |
| SPLEN2005560 | 92.383 | 0 | 0 | 0 | 0 | 0 | 0 |
| SPLEN2005806 | 0 | 0 | 0 | 9.141 | 0 | 0 | 0 |
| SPLEN2006143 | 0 | 0 | 0 | 0 | 0 | 61.507 | 0 |
| SPLEN2006305 | 0 | 0 | 2.317 | 0 | 0 | 0 | 0 |
| SPLEN2006374 | 0 | 0 | 0 | 0 | 10.726 | 0 | 5.299 |
| SPLEN2007498 | 0 | 0 | 0 | 19.560 | 0 | 0 | 0 |
| SPLEN2008460 | 0 | 0 | 0 | 0 | 0 | 36.014 | 0 |
| SPLEN2010195 | 0 | 0 | 1.423 | 0 | 0 | 0 | 0 |
| SPLEN2011737 | 0 | 0 | 0 | 0 | 0 | 0 | 9.083 |
| SPLEN2012523 | 0 | 0 | 0 | 0 | 7.535 | 0 | 0 |
| SPLEN2012611 | 0 | 0 | 0 | 0 | 0 | 0 | 50.050 |
| SPLEN2012800 | 29.977 | 0 | 1.253 | 0 | 0 | 0 | 0 |
| SPLEN2013503 | 0 | 0 | 18.412 | 0 | 0 | 0 | 8.750 |
| SPLEN2013936 | 0 | 0 | 0 | 0 | 0 | 0 | 23.261 |
| SPLEN2014711 | 0 | 0 | 0 | 19.823 | 0 | 72.911 | 0 |
| SPLEN2014911 | 0 | 0 | 0 | 0 | 0 | 29.002 | 0 |
| SPLEN2015310 | 0 | 0 | 0 | 0 | 8.090 | 0 | 0 |
| SPLEN2015730 | 0 | 0 | 51.319 | 0 | 0 | 0 | 0 |
| SPLEN2016743 | 0 | 0 | 21.029 | 0 | 0 | 0 | 0 |
| SPLEN2016972 | 0 | 0 | 0 | 15.156 | 0 | 0 | 0 |
| SPLEN2017351 | 0 | 0 | 0 | 0 | 10.613 | 0 | 0 |
| SPLEN2017620 | 0 | 0 | 18.800 | 0 | 0 | 0 | 0 |
| SPLEN2017918 | 0 | 0 | 3.344 | 0 | 0 | 0 | 0 |
| SPLEN2021194 | 0 | 0 | 12.373 | 0 | 0 | 0 | 0 |
| SPLEN2028466 | 0 | 0 | 0 | 0 | 0 | 0 | 5.769 |
| SPLEN2028914 | 0 | 0 | 10.242 | 0 | 0 | 0 | 0 |
| SPLEN2029912 | 0 | 0 | 0 | 0 | 0 | 44.906 | 0 |
| SPLEN2031547 | 0 | 0 | 5.775 | 0 | 0 | 0 | 0 |
| SPLEN2037722 | 0 | 0 | 0 | 0 | 0 | 45.295 | 0 |
| SPLEN2038473 | 0 | 0 | 50.451 | 0 | 0 | 0 | 0 |
| SPLEN2041306 | 0 | 0 | 28.248 | 0 | 0 | 0 | 0 |
| SPLEN2042920 | 0 | 0 | 0 | 0 | 0 | 74.811 | 0 |
| STOMA1000047 | 0 | 0 | 11.622 | 0 | 0 | 0 | 22.094 |
| STOMA1000189 | 0 | 0 | 0 | 0 | 0 | 0 | 16.356 |
| STOMA2000257 | 0 | 0 | 7.240 | 0 | 0 | 0 | 0 |
| STOMA2000529 | 0 | 50.852 | 0 | 0 | 0 | 0 | 0 |
| STOMA2001389 | 0 | 0 | 21.183 | 0 | 0 | 0 | 0 |
| STOMA2002166 | 0 | 0 | 4.292 | 0 | 4.129 | 0 | 0 |
| SYNOV1000045 | 0 | 0 | 25.765 | 0 | 0 | 0 | 0 |
| SYNOV1000124 | 0 | 0 | 0 | 8.315 | 0 | 0 | 0 |
| SYNOV2001144 | 0 | 0 | 0 | 0 | 0 | 38.881 | 0 |
| SYNOV2001239 | 0 | 0 | 6.905 | 0 | 0 | 0 | 0 |
| SYNOV2001374 | 0 | 0 | 13.436 | 0 | 12.926 | 0 | 0 |
| SYNOV2009172 | 0 | 3.664 | 0 | 0 | 1.836 | 0 | 0 |
| SYNOV4001153 | 0 | 16.331 | 0 | 0 | 0 | 0 | 0 |
| SYNOV4007671 | 0 | 0 | 2.689 | 0 | 0 | 0 | 2.556 |
| TBAES2000059 | 0 | 0 | 5.527 | 0 | 0 | 0 | 5.253 |
| TBAES2001258 | 0 | 0 | 0 | 0 | 0 | 0 | 33.137 |
| TBAES2007428 | 0 | 37.819 | 0 | 0 | 0 | 0 | 0 |
| TBAES2009387 | 0 | 0 | 0 | 0 | 1.886 | 0 | 0 |
| TCERX2000431 | 0 | 0 | 7.705 | 15.701 | 14.824 | 0 | 0 |
| TESOP1000127 | 0 | 0 | 34.899 | 0 | 0 | 0 | 0 |
| TESTI1000023 | 0 | 0 | 3.076 | 6.268 | 0 | 0 | 0 |
| TESTI1000070 | 0 | 0 | 0 | 0 | 0 | 0 | 12.588 |
| TESTI1000168 | 0 | 0 | 11.494 | 0 | 0 | 0 | 0 |
| TESTI2001099 | 0 | 0 | 0 | 3.663 | 0 | 0 | 1.708 |
| TESTI2001120 | 84.853 | 0 | 0 | 0 | 0 | 0 | 0 |
| TESTI2001153 | 0 | 2.804 | 0 | 0 | 0 | 0 | 0 |
| TESTI2001269 | 0 | 3.844 | 0 | 0 | 1.926 | 15.006 | 1.903 |
| TESTI2001829 | 74.526 | 0 | 0 | 0 | 0 | 0 | 0 |
| TESTI2002632 | 0 | 0 | 39.711 | 0 | 0 | 0 | 0 |
| TESTI2002707 | 0 | 0 | 0 | 0 | 0 | 0 | 11.553 |
| TESTI2002866 | 0 | 0 | 0 | 0 | 0 | 0 | 37.093 |
| TESTI2003280 | 0 | 0 | 0 | 0 | 14.474 | 0 | 0 |
| TESTI2003325 | 0 | 0 | 15.522 | 0 | 14.933 | 0 | 0 |
| TESTI2003533 | 0 | 0 | 0 | 0 | 0 | 23.834 | 0 |
| TESTI2003625 | 0 | 0 | 28.073 | 0 | 0 | 0 | 0 |
| TESTI2004031 | 0 | 0 | 0 | 0 | 4.244 | 0 | 1.398 |
| TESTI2004431 | 0 | 0 | 0 | 0 | 0 | 0 | 29.162 |
| TESTI2004712 | 99.261 | 0 | 0 | 0 | 0 | 0 | 0 |
| TESTI2004737 | 0 | 0 | 6.497 | 0 | 0 | 0 | 0 |
| TESTI2005017 | 0 | 0 | 0 | 0 | 11.653 | 0 | 0 |
| TESTI2005112 | 0 | 0 | 0 | 0 | 0 | 0 | 67.827 |
| TESTI2005173 | 0 | 0 | 52.879 | 0 | 0 | 0 | 0 |
| TESTI2005408 | 0 | 24.949 | 0 | 0 | 0 | 0 | 0 |
| TESTI2005743 | 0 | 0 | 0 | 0 | 7.751 | 0 | 0 |
| TESTI2005788 | 0 | 0 | 16.963 | 0 | 0 | 0 | 0 |
| TESTI2005908 | 0 | 11.087 | 0 | 11.769 | 0 | 0 | 0 |
| TESTI2006097 | 0 | 0 | 0 | 78.748 | 0 | 0 | 0 |
| TESTI2006120 | 0 | 0 | 17.800 | 0 | 0 | 0 | 16.919 |
| TESTI2006453 | 0 | 0 | 3.087 | 0 | 0 | 0 | 0 |
| TESTI2006588 | 0 | 0 | 3.971 | 0 | 0 | 0 | 3.775 |
| TESTI2006748 | 0 | 0 | 12.617 | 0 | 0 | 0 | 0 |
| TESTI2007311 | 0 | 0 | 0 | 0 | 0 | 0 | 13.412 |
| TESTI2007998 | 0 | 11.133 | 0 | 11.818 | 0 | 0 | 0 |
| TESTI2008233 | 0 | 0 | 0 | 0 | 0 | 0 | 45.847 |
| TESTI2008240 | 0 | 0 | 0 | 0 | 0 | 0 | 1.744 |
| TESTI2008840 | 0 | 0 | 0 | 0 | 72.965 | 0 | 0 |
| TESTI2009423 | 0 | 0 | 59.311 | 0 | 0 | 0 | 0 |
| TESTI2009520 | 0 | 0 | 0 | 5.605 | 0 | 20.616 | 10.458 |
| TESTI2009577 | 0 | 0 | 0 | 0 | 6.236 | 0 | 0 |
| TESTI2009785 | 0 | 0 | 5.262 | 0 | 0 | 0 | 0 |
| TESTI2009977 | 0 | 0 | 0 | 0 | 0 | 0 | 84.210 |
| TESTI2011605 | 0 | 0 | 13.220 | 0 | 0 | 0 | 0 |
| TESTI2012289 | 0 | 0 | 0 | 0 | 0 | 0 | 30.091 |
| TESTI2012778 | 0 | 0 | 0 | 0 | 1.304 | 0 | 0 |
| TESTI2015213 | 0 | 3.614 | 3.765 | 3.836 | 0 | 0 | 0 |
| TESTI2015626 | 0 | 0 | 2.412 | 0 | 2.321 | 0 | 0 |
| TESTI2017954 | 0 | 9.329 | 0 | 0 | 0 | 0 | 0 |
| TESTI2018462 | 0 | 0 | 10.365 | 0 | 0 | 0 | 0 |
| TESTI2018611 | 92.200 | 0 | 0 | 0 | 0 | 0 | 0 |
| TESTI2024844 | 0 | 0 | 0 | 0 | 4.042 | 0 | 0 |
| TESTI2026215 | 0 | 0 | 0 | 0 | 0 | 0 | 24.148 |
| TESTI2027271 | 0 | 0 | 0 | 39.466 | 0 | 0 | 0 |
| TESTI2033441 | 0 | 0 | 8.049 | 0 | 0 | 0 | 0 |
| TESTI2034916 | 0 | 0 | 0 | 56.816 | 0 | 0 | 0 |
| TESTI2034940 | 0 | 0 | 1.911 | 3.894 | 1.838 | 0 | 0.908 |
| TESTI2040094 | 0 | 0 | 0 | 91.958 | 0 | 0 | 0 |
| TESTI2046456 | 0 | 0 | 1.698 | 0 | 0 | 0 | 3.227 |
| TESTI2051177 | 0 | 0 | 0 | 0 | 84.370 | 0 | 0 |
| TESTI4000014 | 0 | 0 | 0.655 | 0 | 0.630 | 4.909 | 1.245 |
| TESTI4002799 | 38.761 | 0 | 0 | 0 | 0 | 0 | 0 |
| TESTI4003703 | 0 | 0 | 16.229 | 0 | 0 | 0 | 0 |
| TESTI4010851 | 0 | 3.849 | 0 | 0 | 0 | 0 | 0 |
| TESTI4013441 | 0 | 2.209 | 0.575 | 0 | 0.553 | 0 | 0.547 |
| TESTI4013474 | 0 | 0 | 3.790 | 0 | 0 | 0 | 0 |
| TESTI4037156 | 0 | 0 | 2.030 | 0 | 0.976 | 0 | 0 |
| TESTI4043140 | 0 | 0 | 10.170 | 0 | 0 | 0 | 0 |
| TESTI4044704 | 0 | 0 | 18.694 | 0 | 0 | 0 | 0 |
| THYMU1000016 | 0 | 0 | 0 | 0 | 2.727 | 0 | 0 |
| THYMU1000041 | 0 | 0 | 0 | 0 | 0 | 0 | 8.290 |
| THYMU1000109 | 0 | 0 | 2.887 | 0 | 0 | 0 | 2.744 |
| THYMU1000359 | 0 | 0 | 0 | 0 | 0 | 0 | 1.869 |
| THYMU1000374 | 0 | 0 | 0 | 0 | 0 | 0 | 5.222 |
| THYMU1000394 | 0 | 0 | 18.030 | 0 | 0 | 0 | 0 |
| THYMU1000428 | 0 | 0 | 2.085 | 0 | 0 | 0 | 1.982 |
| THYMU1000473 | 0 | 0 | 0 | 0 | 0 | 91.440 | 0 |
| THYMU1000499 | 0 | 40.188 | 0 | 0 | 20.139 | 0 | 0 |
| THYMU2000057 | 0 | 0 | 4.974 | 0 | 0 | 0 | 0 |
| THYMU2000300 | 0 | 0 | 0 | 21.836 | 0 | 0 | 0 |
| THYMU2000369 | 0 | 0 | 0 | 0 | 0 | 0 | 62.676 |
| THYMU2000436 | 0 | 0 | 6.759 | 0 | 6.502 | 0 | 0 |
| THYMU2000702 | 0 | 0 | 0 | 0 | 0 | 86.018 | 0 |
| THYMU2000775 | 0 | 0 | 34.340 | 0 | 0 | 0 | 0 |
| THYMU2000946 | 0 | 0 | 0 | 10.814 | 0 | 0 | 0 |
| THYMU2001018 | 0 | 0 | 33.126 | 0 | 0 | 0 | 0 |
| THYMU2002080 | 0 | 0 | 54.584 | 0 | 0 | 0 | 0 |
| THYMU2002583 | 0 | 3.649 | 0 | 3.874 | 0 | 0 | 1.807 |
| THYMU2002745 | 0 | 0 | 0 | 0 | 13.509 | 0 | 0 |
| THYMU2003012 | 0 | 0 | 0 | 6.550 | 0 | 0 | 0 |
| THYMU2003446 | 0 | 0 | 8.748 | 0 | 0 | 0 | 0 |
| THYMU2003891 | 0 | 0 | 1.070 | 0 | 0 | 0 | 0 |
| THYMU2003981 | 0 | 0 | 1.915 | 0 | 0 | 0 | 0 |
| THYMU2004410 | 0 | 0 | 14.055 | 0 | 0 | 0 | 0 |
| THYMU2004916 | 0 | 0 | 11.447 | 0 | 0 | 0 | 5.440 |
| THYMU2006505 | 0 | 0 | 0 | 10.203 | 9.634 | 0 | 0 |
| THYMU2007415 | 0 | 0 | 8.875 | 0 | 0 | 0 | 0 |
| THYMU2007467 | 0 | 0 | 14.815 | 0 | 0 | 0 | 0 |
| THYMU2007729 | 0 | 0 | 17.926 | 0 | 0 | 0 | 17.039 |
| THYMU2009658 | 0 | 0 | 0 | 0 | 3.708 | 0 | 0 |
| THYMU2009792 | 0 | 0 | 0 | 0 | 0 | 76.990 | 0 |
| THYMU2012401 | 0 | 0 | 0 | 0 | 0 | 0 | 80.699 |
| THYMU2013136 | 0 | 5.759 | 0 | 0 | 0 | 0 | 0 |
| THYMU2013170 | 0 | 0 | 0 | 0 | 80.886 | 0 | 0 |
| THYMU2013916 | 0 | 7.810 | 4.068 | 0 | 0 | 0 | 7.734 |
| THYMU2015775 | 0 | 0 | 81.477 | 0 | 0 | 0 | 0 |
| THYMU2022660 | 0 | 0 | 0 | 0 | 80.886 | 0 | 0 |
| THYMU2027125 | 0 | 0 | 0 | 0 | 0.632 | 0 | 0 |
| THYMU2032014 | 0 | 0 | 0 | 0 | 0 | 0 | 57.532 |
| THYMU2032696 | 92.236 | 0 | 0 | 0 | 0 | 0 | 0 |
| THYMU2033059 | 0 | 0 | 81.477 | 0 | 0 | 0 | 0 |
| THYMU3005629 | 0 | 0 | 55.420 | 0 | 0 | 0 | 0 |
| THYMU3015759 | 0 | 0 | 0 | 8.415 | 0 | 0 | 0 |
| THYMU3032867 | 0 | 0 | 0 | 0 | 0 | 0 | 44.871 |
| THYMU3033626 | 0 | 0 | 20.615 | 0 | 0 | 0 | 0 |
| THYMU3034671 | 0 | 0 | 8.747 | 0 | 0 | 0 | 0 |
| THYMU3041428 | 0 | 0 | 0.935 | 0 | 0 | 0 | 0 |
| TKIDN2002738 | 0 | 2.953 | 0 | 0 | 0 | 0 | 1.462 |
| TKIDN2005487 | 63.052 | 0 | 2.635 | 0 | 0 | 0 | 0 |
| TKIDN2011289 | 0 | 0 | 0 | 0 | 21.094 | 0 | 0 |
| TRACH1000030 | 0 | 0 | 2.530 | 5.156 | 0 | 0 | 4.810 |
| TRACH1000063 | 0 | 0 | 0 | 0 | 7.589 | 0 | 0 |
| TRACH2000045 | 0 | 0 | 0 | 0 | 0 | 0 | 9.587 |
| TRACH2000079 | 0 | 7.629 | 0 | 0 | 0 | 0 | 0 |
| TRACH2000250 | 0 | 0 | 0 | 0 | 0 | 0 | 64.083 |
| TRACH2000321 | 0 | 3.460 | 1.802 | 7.344 | 0 | 0 | 1.713 |
| TRACH2000461 | 0 | 0 | 6.289 | 0 | 0 | 0 | 0 |
| TRACH2000702 | 0 | 0 | 14.400 | 0 | 0 | 0 | 0 |
| TRACH2000780 | 0 | 0 | 3.206 | 0 | 0 | 0 | 3.047 |
| TRACH2000926 | 0 | 0 | 10.233 | 0 | 0 | 0 | 0 |
| TRACH2000959 | 0 | 0 | 0.895 | 0 | 0 | 0 | 0 |
| TRACH2000972 | 0 | 0 | 0 | 0 | 0 | 0 | 4.132 |
| TRACH2001021 | 0 | 0 | 1.536 | 0 | 0 | 0 | 0 |
| TRACH2001101 | 0 | 0 | 19.498 | 0 | 0 | 0 | 9.267 |
| TRACH2001289 | 0 | 2.393 | 0 | 0 | 1.199 | 0 | 1.185 |
| TRACH2001314 | 0 | 0 | 0 | 0 | 6.449 | 0 | 0 |
| TRACH2001341 | 0 | 0 | 23.377 | 0 | 0 | 0 | 0 |
| TRACH2001432 | 0 | 0 | 0 | 8.314 | 0 | 0 | 0 |
| TRACH2001478 | 0 | 0 | 0 | 0 | 0 | 15.958 | 0 |
| TRACH2001684 | 0 | 0 | 0 | 0 | 0 | 0 | 62.100 |
| TRACH2001810 | 0 | 5.468 | 0 | 0 | 5.480 | 0 | 0 |
| TRACH2002092 | 0 | 19.765 | 0 | 0 | 0 | 0 | 0 |
| TRACH2002537 | 0 | 13.060 | 0 | 13.863 | 0 | 0 | 3.233 |
| TRACH2002664 | 0 | 0 | 5.582 | 0 | 0 | 0 | 0 |
| TRACH2002847 | 0 | 0 | 0 | 33.924 | 0 | 0 | 0 |
| TRACH2003272 | 0 | 0 | 0 | 0 | 0 | 0 | 12.917 |
| TRACH2003347 | 0 | 0 | 5.827 | 0 | 5.606 | 0 | 0 |
| TRACH2005800 | 0 | 0 | 0 | 35.963 | 0 | 0 | 0 |
| TRACH2005811 | 0 | 0 | 3.868 | 0 | 0 | 0 | 0 |
| TRACH2007399 | 0 | 3.317 | 0 | 7.041 | 0 | 0 | 0 |
| TRACH2009291 | 0 | 0 | 76.605 | 0 | 0 | 0 | 0 |
| TRACH2009851 | 0 | 0 | 55.151 | 0 | 0 | 0 | 0 |
| TRACH2010634 | 0 | 0 | 0 | 0 | 61.166 | 0 | 0 |
| TRACH2011302 | 0 | 86.275 | 0 | 0 | 0 | 0 | 0 |
| TRACH2012376 | 0 | 0 | 0 | 20.060 | 0 | 0 | 0 |
| TRACH2013081 | 0 | 0 | 0 | 86.967 | 0 | 0 | 0 |
| TRACH2014077 | 0 | 0 | 0 | 0 | 12.003 | 0 | 23.718 |
| TRACH2014124 | 0 | 0 | 3.786 | 0 | 0 | 0 | 0 |
| TRACH2014442 | 0 | 31.705 | 0 | 0 | 0 | 0 | 0 |
| TRACH2014997 | 0 | 24.910 | 0 | 0 | 0 | 0 | 0 |
| TRACH2015654 | 0 | 0 | 76.605 | 0 | 0 | 0 | 0 |
| TRACH2016317 | 0 | 0 | 0 | 0 | 82.533 | 0 | 0 |
| TRACH2018317 | 0 | 0 | 10.745 | 0 | 0 | 0 | 0 |
| TRACH2018446 | 0 | 0 | 15.339 | 0 | 0 | 0 | 0 |
| TRACH2019672 | 0 | 0 | 76.605 | 0 | 0 | 0 | 0 |
| TRACH2025535 | 0 | 0 | 0 | 4.339 | 4.097 | 0 | 0 |
| TRACH3000692 | 0 | 0 | 0 | 0 | 0 | 0 | 16.340 |
| TRACH3002192 | 0 | 4.391 | 0 | 0 | 2.201 | 0 | 0 |
| TRACH3004068 | 0 | 0 | 5.227 | 0 | 0 | 0 | 0 |
| TRACH3008093 | 0 | 34.493 | 0 | 18.307 | 0 | 0 | 0 |
| TRACH3022198 | 0 | 0 | 30.484 | 0 | 0 | 0 | 0 |
| UMVEN1000186 | 0 | 1.860 | 0 | 0 | 0 | 0 | 0 |
| UMVEN2000354 | 0 | 0 | 0 | 0 | 2.906 | 0 | 0 |
| UTERU1000384 | 0 | 0 | 0 | 0 | 25.149 | 0 | 0 |
| UTERU2000095 | 0 | 2.914 | 0 | 3.093 | 0 | 0 | 0 |
| UTERU2000263 | 0 | 0 | 0 | 3.830 | 0 | 0 | 0 |
| UTERU2000537 | 0 | 0 | 0 | 0 | 0 | 0 | 7.887 |
| UTERU2000696 | 0 | 0 | 0 | 21.268 | 0 | 0 | 0 |
| UTERU2000925 | 0 | 0 | 38.195 | 0 | 0 | 0 | 0 |
| UTERU2001658 | 0 | 0 | 32.488 | 0 | 0 | 0 | 0 |
| UTERU2002410 | 51.532 | 0 | 0 | 0 | 0 | 0 | 0 |
| UTERU2003057 | 0 | 0 | 0 | 19.331 | 0 | 0 | 0 |
| UTERU2004037 | 0 | 15.438 | 0 | 0 | 0 | 0 | 0 |
| UTERU2004197 | 0 | 0 | 0 | 15.029 | 0 | 0 | 0 |
| UTERU2004807 | 0 | 0 | 7.369 | 22.526 | 0 | 0 | 0 |
| UTERU2005354 | 0 | 0 | 0 | 0 | 7.718 | 0 | 0 |
| UTERU2005593 | 0 | 0 | 0 | 0 | 3.960 | 0 | 0 |
| UTERU2005621 | 0 | 0 | 8.268 | 0 | 0 | 0 | 0 |
| UTERU2006643 | 0 | 59.791 | 0 | 0 | 0 | 0 | 0 |
| UTERU2007724 | 0 | 46.260 | 0 | 0 | 0 | 0 | 0 |
| UTERU2008085 | 0 | 0 | 0.662 | 0 | 3.505 | 0 | 1.574 |
| UTERU2008747 | 0 | 0 | 0 | 0 | 31.185 | 0 | 0 |
| UTERU2008938 | 0 | 0 | 9.027 | 0 | 0 | 0 | 0 |
| UTERU2009972 | 0 | 0 | 0 | 0 | 0 | 0 | 12.351 |
| UTERU2010417 | 0 | 0 | 0 | 2.356 | 0 | 0 | 1.099 |
| UTERU2012286 | 0 | 0 | 9.292 | 0 | 8.940 | 0 | 0 |
| UTERU2012703 | 0 | 0 | 75.461 | 0 | 0 | 0 | 0 |
| UTERU2012767 | 0 | 0 | 0 | 0 | 18.790 | 0 | 0 |
| UTERU2014024 | 0 | 0 | 0 | 0 | 0 | 0 | 45.181 |
| UTERU2014167 | 0 | 0 | 0 | 6.812 | 0 | 0 | 3.177 |
| UTERU2015653 | 0 | 0 | 0 | 4.360 | 0 | 0 | 0 |
| UTERU2016799 | 0 | 0 | 0 | 0 | 20.520 | 0 | 0 |
| UTERU2023175 | 0 | 0 | 0 | 0 | 5.253 | 0 | 5.190 |

Genes whose expression levels were negligibly low in each of the above-described libraries are shown with their clone names below.

3NB691000018, 3NB691000085, 3NB691000129, 3NB691000191, 3NB692000012, 3NB692000029, 3NB692000102, 3NB692000154, 3NB692000276, 3NB692000305, 3NB692000429, 3NB692000484, 3NB692000529, 3NB692000973, 3NB692001034, 3NB692001040, 3NB692001123, 3NB692001267, 3NB692001339, 3NB692001349, 3NB692001366, 3NB692001408, 3NB692001433, 3NB692001471, 3NB692001496, 3NB692001501, 3NB692001507, 3NB692001519, 3NB692001520, 3NB692001528, 3NB692001538, 3NB692001548, 3NB692001557, 3NB692001719, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064, ADIPS2000069,

ADIPS2000088, ADIPS2000245, ADIPS2000425, ADRGL1000002, ADRGL1000018, ADRGL1000033, ADRGL1000038, ADRGL1000065, ADRGL1000067, ADRGL1000111, ADRGL1000144, ADRGL1000147, ADRGL1000160, ADRGL1000165, ADRGL1000182, ADRGL2000006, ADRGL2000042, ADRGL2000056, ADRGL2000064, ADRGL2000074, ADRGL2000085, ADRGL2000097, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260,

ADRGL2002392, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974,

ADRGL2008023, ADRGL2008331, ADRGL2008337, ADRGL2008535, ADRGL2008966, ADRGL2009146, ADRGL2009273, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012179, AHMSC1000138, ASTRO1000009, ASTRO1000018, ASTRO1000029, ASTRO1000096, ASTRO2000011, ASTRO2000095, ASTRO2000141, ASTRO2000191, ASTRO2000278, ASTRO2000372, ASTRO2000381, ASTRO2000403, ASTRO2000417, ASTRO2000482, ASTRO2000533, ASTRO2000653, ASTRO2000662, ASTRO2000725, ASTRO2001001, ASTRO2001008, ASTRO2001088, ASTRO2001107, ASTRO2001122, ASTRO2001227, ASTRO2001249, ASTRO2001480, ASTRO2002024, ASTRO2002035, ASTRO2002064,

ASTRO2002202, ASTRO2002571, ASTRO2002632, ASTRO2002659, ASTRO2002693, ASTRO2002720, ASTRO2002733, ASTRO2002743, ASTRO2002842, ASTRO2003212, ASTRO2003316, ASTRO2003461, ASTRO2003574, ASTRO2003581, ASTRO2003740, ASTRO2003840, ASTRO2003925, ASTRO2003960, ASTRO2004032, ASTRO2004051, ASTRO2004177, ASTRO2004217, ASTRO2004297, ASTRO2004584, ASTRO2004628, ASTRO2004751, ASTRO2004877, ASTRO2004974, ASTRO2005008, ASTRO2005081, ASTRO2005096, ASTRO2005242, ASTRO2005273, ASTRO2005343, ASTRO2005360, ASTRO2005413, ASTRO2005423, ASTRO2005553, ASTRO2005557, ASTRO2005575, ASTRO2005593, ASTRO2005726, ASTRO2005863, ASTRO2005896, ASTRO2005902, ASTRO2006356, ASTRO2006475, ASTRO2006732, ASTRO2006747, ASTRO2006920,

ASTRO2006952, ASTRO2007221, ASTRO2007377, ASTRO2007515, ASTRO2007578, ASTRO2007643, ASTRO2007666, ASTRO2007948, ASTRO2007956, ASTRO2007962, ASTRO2008040, ASTRO2008216, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561,

ASTRO2014576, ASTRO2014863, ASTRO2014923, ASTRO2014961, ASTRO2015162, ASTRO2015185, ASTRO2015328, ASTRO2015529, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018373, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000193, BGGI11000285, BGGI12000022, BGGI12000054, BGGI12000067, BGGI12000161, BGGI12000544, BGGI12000684, BGGI12000693, BGGI12001002, BGGI12001075, BGGI12001241, BGGI12001247, BGGI12001714, BLADE1000176, BLADE2000181, BLADE2000256, BLADE2000340, BLADE2000389, BLADE2000463, BLADE2000492,

BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006849, BLADE2006947, BLADE2007033, BLADE2007043, BLADE2007666, BLADE2007722, BLADE2007735, BLADE2007744,

BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000137, BNGH41000169, BNGH41000177, BNGH41000190, BNGH41000216, BNGH41000235, BNGH42000132, BNGH42000405, BNGH42000532, BNGH42000675, BNGH42000791, BNGH42000815, BNGH42000994, BNGH42001247, BNGH42001406, BNGH42001576, BNGH42001724, BNGH42002168, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504,

BNGH42005968, BNGH42006135, BNGH42006226, BNGH42006234, BNGH42007037, BNGH42007366, BNGH42007460, BNGH42007594, BNGH42007798, BNGH42008199, BNGH42008510, BNGH42008603, BNGH42008649, BNGH42008743, BNGH42008832, BNGH42008850, BNGH42008943, BNGH42009021, BNGH42009025, BRACE1000020, BRACE1000042, BRACE1000047, BRACE1000051, BRACE1000065, BRACE1000070, BRACE1000073, BRACE1000084, BRACE1000087, BRACE1000092, BRACE1000093, BRACE1000101, BRACE1000114, BRACE1000115, BRACE1000124, BRACE1000132, BRACE1000166, BRACE1000169, BRACE1000187, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000148,

BRACE2000183, BRACE2000229, BRACE2000232, BRACE2000256, BRACE2000266, BRACE2000267, BRACE2000280, BRACE2000300, BRACE2000307, BRACE2000331, BRACE2000332, BRACE2000347, BRACE2000351, BRACE2000368, BRACE2000392, BRACE2000441, BRACE2000487, BRACE2000489, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000753, BRACE2000815, BRACE2000864, BRACE2000885, BRACE2000893, BRACE2000905, BRACE2000936, BRACE2000947, BRACE2000965, BRACE2000975, BRACE2000988, BRACE2001017, BRACE2001065, BRACE2001070, BRACE2001094, BRACE2001107, BRACE2001143, BRACE2001151,

BRACE2001154, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001508, BRACE2001534, BRACE2001543, BRACE2001564, BRACE2001588, BRACE2001607, BRACE2001626, BRACE2001673, BRACE2001677, BRACE2001705, BRACE2001709, BRACE2001711, BRACE2001726, BRACE2001733, BRACE2001737, BRACE2001742, BRACE2001777, BRACE2001779, BRACE2001834, BRACE2001859, BRACE2001865, BRACE2001872, BRACE2001881, BRACE2001898, BRACE2001923, BRACE2001942, BRACE2001944,

BRACE2001954, BRACE2001972, BRACE2001973, BRACE2002026, BRACE2002034, BRACE2002050, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002661, BRACE2002676, BRACE2002677, BRACE2002682, BRACE2002695, BRACE2002707, BRACE2002736, BRACE2002752, BRACE2002755, BRACE2002762, BRACE2002772, BRACE2002792,

BRACE2002796, BRACE2002803, BRACE2002807, BRACE2002812, BRACE2002834, BRACE2002849, BRACE2002857, BRACE2002861, BRACE2002884, BRACE2002896, BRACE2002948, BRACE2003037, BRACE2003078, BRACE2003097, BRACE2003110, BRACE2003168, BRACE2003199, BRACE2003210, BRACE2003269, BRACE2003285, BRACE2003304, BRACE2003319, BRACE2003398, BRACE2003420, BRACE2003428, BRACE2003431, BRACE2003512, BRACE2003516, BRACE2003539, BRACE2003540, BRACE2003594, BRACE2003598, BRACE2003609, BRACE2003614, BRACE2003628, BRACE2003639, BRACE2003700, BRACE2003713, BRACE2003766, BRACE2003785, BRACE2003788, BRACE2003800, BRACE2003802, BRACE2003825, BRACE2003845, BRACE2003847, BRACE2003848, BRACE2003873, BRACE2003885, BRACE2003887,

BRACE2003905, BRACE2003928, BRACE2003944, BRACE2003954, BRACE2003982, BRACE2003987, BRACE2005034, BRACE2005047, BRACE2005063, BRACE2005083, BRACE2005087, BRACE2005090, BRACE2005118, BRACE2005124, BRACE2005138, BRACE2005151, BRACE2005160, BRACE2005169, BRACE2005189, BRACE2005193, BRACE2005196, BRACE2005198, BRACE2005216, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005433, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005719, BRACE2005731,

BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006072, BRACE2006075, BRACE2006084, BRACE2006102, BRACE2.006105, BRACE2006122, BRACE2006137, BRACE2006143, BRACE2006162, BRACE2006167, BRACE2006174, BRACE2006240, BRACE2006245, BRACE2006249, BRACE2006264, BRACE2006274, BRACE2006281, BRACE2006298, BRACE2006319, BRACE2006344, BRACE2006354, BRACE2006363, BRACE2006378, BRACE2006380, BRACE2006393, BRACE2006413, BRACE2006417, BRACE2006453, BRACE2006459, BRACE2006488, BRACE2006534,

BRACE2006537, BRACE2006540, BRACE2006547, BRACE2006564, BRACE2006587, BRACE2006598, BRACE2006636, BRACE2006685, BRACE2006703, BRACE2006743, BRACE2006753, BRACE2006783, BRACE2006821, BRACE2006833, BRACE2006859, BRACE2006909, BRACE2006924, BRACE2006935, BRACE2006937, BRACE2006944, BRACE2006951, BRACE2006982, BRACE2006986, BRACE2007013, BRACE2007064, BRACE2007068, BRACE2007087, BRACE2007109, BRACE2007138, BRACE2007174, BRACE2007191, BRACE2007197, BRACE2007201, BRACE2007203, BRACE2007232, BRACE2007254, BRACE2007262, BRACE2007274, BRACE2007281, BRACE2007295, BRACE2007358, BRACE2007396, BRACE2007398, BRACE2007411, BRACE2007447, BRACE2007477, BRACE2007502, BRACE2007503, BRACE2007522, BRACE2007527,

BRACE2007538, BRACE2007563, BRACE2007567, BRACE2007621, BRACE2007640, BRACE2007641, BRACE2007646, BRACE2007663, BRACE2007671, BRACE2007682, BRACE2007685, BRACE2007708, BRACE2007727, BRACE2007749, BRACE2007760, BRACE2007761, BRACE2007764, BRACE2007767, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008102, BRACE2008114, BRACE2008144, BRACE2008172, BRACE2008216, BRACE2008218, BRACE2008276, BRACE2008295, BRACE2008308, BRACE2008336, BRACE2008358, BRACE2008361,

BRACE2008399, BRACE2008401, BRACE2008402, BRACE2008410, BRACE2008420, BRACE2008443, BRACE2008473, BRACE2008480, BRACE2008481, BRACE2008488, BRACE2008500, BRACE2008553, BRACE2008594, BRACE2008604, BRACE2008615, BRACE2008622, BRACE2008629, BRACE2008653, BRACE2008655, BRACE2008656, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008941, BRACE2008948, BRACE2008960, BRACE2008999, BRACE2009014, BRACE2009016, BRACE2009031, BRACE2009044, BRACE2009045, BRACE2009053, BRACE2009122, BRACE2009131, BRACE2009185, BRACE2009188, BRACE2009212, BRACE2009221, BRACE2009235, BRACE2009238,

BRACE2009255, BRACE2009270, BRACE2009272, BRACE2009274, BRACE2009275, BRACE2009291, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009517, BRACE2009533, BRACE2009558, BRACE2009566, BRACE2009591, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813,

BRACE2010828, BRACE2010837, BRACE2010888, BRACE2010937, BRACE2010983, BRACE2011007, BRACE2011025, BRACE2011183, BRACE2011199, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012062, BRACE2012185, BRACE2012222, BRACE2012232, BRACE2012291, BRACE2012317, BRACE2012510, BRACE2012528, BRACE2012625, BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257,

BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017397, BRACE2017410, BRACE2017438, BRACE2017574, BRACE2017580, BRACE2017587,

BRACE2017720, BRACE2017844, BRACE2017872, BRACE2017929, BRACE2017934, BRACE2017985, BRACE2017992, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982,

BRACE2021014, BRACE2021143, BRACE2021148, BRACE2021245, BRACE2021254, BRACE2021264, BRACE2021344, BRACE2021433, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022638, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610,

BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849,

BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830,

BRACE2036834, BRACE2036900, BRACE2037132, BRACE2037294, BRACE2037295, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873,

BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113,

BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004205, BRACE3004358, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499,

BRACE3005760, BRACE3005870, BRACE3005903, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708,

BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010438, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020,

BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153,

BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537,

BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495,

BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734,

BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157,

BRAMY1000173, BRAMY2000021, BRAMY2000025, BRAMY2000052, BRAMY2000076, BRAMY2000086, BRAMY2000181, BRAMY2000196, BRAMY2000231, BRAMY2000277, BRAMY2000411, BRAMY2000508, BRAMY2000562, BRAMY2000585, BRAMY2000653, BRAMY2000814, BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037,

BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004363, BRAMY2004387, BRAMY2004492, BRAMY2004521, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411,

BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2010068, BRAMY2010135, BRAMY2010145,

BRAMY2010171, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012536, BRAMY2012555, BRAMY2012568, BRAMY2012581, BRAMY2012691, BRAMY2012721, BRAMY2012731, BRAMY2012776, BRAMY2012880, BRAMY2012881,

BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015925, BRAMY2016002, BRAMY2016009, BRAMY2016020, BRAMY2016070, BRAMY2016251, BRAMY2016327, BRAMY2016386, BRAMY2016706, BRAMY2016771, BRAMY2016892, BRAMY2016938, BRAMY2016953,

BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019600, BRAMY2019643, BRAMY2019710, BRAMY2019963, BRAMY2019985, BRAMY2019989, BRAMY2020050, BRAMY2020058, BRAMY2020106, BRAMY2020270, BRAMY2020354, BRAMY2020355, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276,

BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2023939, BRAMY2024073, BRAMY2024312, BRAMY2024374, BRAMY2024390, BRAMY2024416, BRAMY2024449,

BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026685, BRAMY2026713, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717,

BRAMY2027739, BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028740, BRAMY2028783, BRAMY2028796, BRAMY2028856, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2032014, BRAMY2032087, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003,

BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484,

BRAMY2038516, BRAMY2038846, BRAMY2038887, BRAMY2038904, BRAMY2039287, BRAMY2039300, BRAMY2039314, BRAMY2039341, BRAMY2039695, BRAMY2039728, BRAMY2039872, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299,

BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3002991, BRAMY3003026, BRAMY3003109, BRAMY3003205, BRAMY3003310, BRAMY3003566, BRAMY3003723, BRAMY3004126, BRAMY3004224, BRAMY3004672,

BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652,

BRAMY4001863, BRAMY4001913, BRAMY4002575, BRAMY4002628, BRASW1000053, BRASW1000125, BRAWH1000001, BRAWH1000002, BRAWH1000007, BRAWH1000037, BRAWH1000045, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000443, BRAWH2000471, BRAWH2000476, BRAWH2000482, BRAWH2000488, BRAWH2000494, BRAWH2000500, BRAWH2000503, BRAWH2000554,

BRAWH2000595, BRAWH2000643, BRAWH2000651, BRAWH2000682, BRAWH2000686, BRAWH2000752, BRAWH2000839, BRAWH2000859, BRAWH2000866, BRAWH2000892, BRAWH2000901, BRAWH2000924, BRAWH2000926, BRAWH2000984, BRAWH2001019, BRAWH2001037, BRAWH2001052, BRAWH2001103, BRAWH2001129, BRAWH2001137, BRAWH2001141, BRAWH2001159, BRAWH2001171, BRAWH2001186, BRAWH2001203, BRAWH2001238, BRAWH2001255, BRAWH2001321, BRAWH2001355, BRAWH2001378, BRAWH2001395, BRAWH2001406, BRAWH2001412, BRAWH2001418, BRAWH2001436, BRAWH2001438, BRAWH2001439, BRAWH2001459, BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686,

BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003135, BRAWH2003240, BRAWH2003307, BRAWH2003355, BRAWH2003366, BRAWH2003662, BRAWH2003689, BRAWH2003832, BRAWH2003948, BRAWH2003964, BRAWH2004044, BRAWH2004068, BRAWH2004095, BRAWH2004136, BRAWH2004217, BRAWH2004580, BRAWH2004731, BRAWH2004779,

BRAWH2004842, BRAWH2004884, BRAWH2004923, BRAWH2005008, BRAWH2005074, BRAWH2005225, BRAWH2005315, BRAWH2005517, BRAWH2005524, BRAWH2005533, BRAWH2005578, BRAWH2005661, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2007069, BRAWH2007308, BRAWH2007406, BRAWH2007550, BRAWH2007570, BRAWH2007591, BRAWH2007605, BRAWH2007658, BRAWH2007664, BRAWH2007800, BRAWH2007808, BRAWH2007825, BRAWH2007862, BRAWH2007890, BRAWH2008058, BRAWH2008128, BRAWH2008219, BRAWH2008292, BRAWH2008297, BRAWH2008366, BRAWH2008380,

BRAWH2008540, BRAWH2008577, BRAWH2008706, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009304, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010536, BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400, BRAWH2011762,

BRAWH2011795, BRAWH2011796, BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014234, BRAWH2014379, BRAWH2014414, BRAWH2014473, BRAWH2014511, BRAWH2014645, BRAWH2014662, BRAWH2014717, BRAWH2014729, BRAWH2014876, BRAWH2014934, BRAWH2014954, BRAWH2015247, BRAWH2015375, BRAWH2015385, BRAWH2015595, BRAWH2015728, BRAWH2015853, BRAWH2015866,

BRAWH2016028, BRAWH2016106, BRAWH2016160, BRAWH2016166, BRAWH2016209, BRAWH2016221, BRAWH2016223, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017542, BRAWH2017635, BRAWH2017685, BRAWH2017913, BRAWH2018206, BRAWH2018267, BRAWH2018282, BRAWH2018317, BRAWH2018506, BRAWH2018514, BRAWH2018527, BRAWH2018601, BRAWH2018745, BRAWH2018875, BRAWH2019053, BRAWH2019055, BRAWH2019198, BRAWH3000078, BRAWH3000088, BRAWH3000100,

BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002574, BRAWH3002600, BRAWH3002819, BRAWH3003019, BRAWH3003136, BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005146, BRAWH3005292, BRAWH3005300,

BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197,

BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021545, BRAWH3021574, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023274, BRAWH3023421, BRAWH3024231,

BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114,

BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN1000168, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000763, BRCAN2000832, BRCAN2000923, BRCAN2001223, BRCAN2001866, BRCAN2002416, BRCAN2002473, BRCAN2002562,

BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2009432, BRCAN2010374, BRCAN2010376,

BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653,

BRCAN2019773, BRCAN2019907, BRCAN2020234, BRCAN2020331, BRCAN2020412, BRCAN2020467, BRCAN2020710, BRCAN2020880, BRCAN2021024, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2025009, BRCAN2025093, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028040, BRCAN2028319, BRCAN2028338, BRCAN2028355, BRCAN2028378, BRCAN2028460, BRCAN2028545, BRCAN2028634, BRCAN2028702, BRCOC1000040, BRCOC1000087,

BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003187, BRCOC2003213, BRCOC2003513, BRCOC2003732, BRCOC2003740, BRCOC2004036, BRCOC200.4175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769,

BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010510, BRCOC2010730, BRCOC2010980, BRCOC2010994, BRCOC2011010, BRCOC2011398, BRCOC2011418, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019910, BRCOC2019934,

BRCOC2020122, BRCOC2020142, BRHIP1000058, BRHIP1000072, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000621, BRHIP2000691, BRHIP2000826, BRHIP2000920, BRHIP2001074, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355,

BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007741, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011491,

BRHIP2011508, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387, BRHIP2014954, BRHIP2015153, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553,

BRHIP2017642, BRHIP2017714, BRHIP2017780, BRHIP2017945, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020573, BRHIP2020622, BRHIP2020695, BRHIP2020743, BRHIP2020799, BRHIP2020842, BRHIP2020859, BRHIP2020930, BRHIP2021102, BRHIP2021289, BRHIP2021495, BRHIP2021615, BRHIP2021744, BRHIP2021762, BRHIP2021858, BRHIP2021929, BRHIP2022006, BRHIP2022054, BRHIP2022221, BRHIP2022228, BRHIP2022298, BRHIP2022326, BRHIP2022467, BRHIP2022708,

BRHIP2022986, BRHIP2023071, BRHIP2023229, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024549, BRHIP2024742, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176,

BRHIP2029283, BRHIP2029393, BRHIP2029529, BRHIP2029643, BRHIP2029663, BRHIP3000017, BRHIP3000060, BRHIP3000070, BRHIP3000081, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710,

BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006786, BRHIP3006950, BRHIP3007172, BRHIP3007183, BRHIP3007195, BRHIP3007223, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181,

BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3018784, BRHIP3019643, BRHIP3019824, BRHIP3019880, BRHIP3019956, BRHIP3020046,

BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000498, BRSSN2000518, BRSSN2000561, BRSSN2000566,

BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2001869, BRSSN2002857, BRSSN2002859, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395,

BRSSN2009627, BRSSN2009630, BRSSN2009702, BRSSN2010019, BRSSN2010110, BRSSN2010587, BRSSN2010596, BRSSN2010830, BRSSN2011262, BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014610, BRSSN2014636, BRSSN2014685, BRSSN2015199, BRSSN2015238, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768,

BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000083, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457,

BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451, BRSTN2013354, BRSTN2013502, BRSTN2013931, BRSTN2014490, BRSTN2014633, BRSTN2014751, BRSTN2014770, BRSTN2015015, BRSTN2015889, BRSTN2015978, BRSTN2016066, BRSTN2016335, BRSTN2016678, BRSTN2016918, BRSTN2016992, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063,

BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831,

BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2007998, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129,

BRTHA2012183, BRTHA2012189, BRTHA2012351, BRTHA2012392, BRTHA2012466, BRTHA2012555, BRTHA2012562, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016765, BRTHA2016918, BRTHA2017178, BRTHA2017353, BRTHA2017364, BRTHA2017972, BRTHA2017985,

BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027250, BRTHA2027546, BRTHA2028297, BRTHA2029969, BRTHA2030036, BRTHA2030213, BRTHA2031517,

BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003490,

BRTHA3003704, BRTHA3003736, BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010717, BRTHA3011149, BRTHA3011187, BRTHA3011194, BRTHA3011229, BRTHA3011265, BRTHA3011361, BRTHA3011510, BRTHA3011892,

BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3021971, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507,

BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000252, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, CHONS2002829, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318,

COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000009, CTONG1000010, CTONG1000020, CTONG1000022, CTONG1000040, CTONG1000056, CTONG1000062, CTONG1000086, CTONG1000088, CTONG1000093, CTONG1000097, CTONG1000113, CTONG1000137, CTONG1000165, CTONG1000180, CTONG1000181, CTONG1000218, CTONG1000220, CTONG1000241, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034,

CTONG2000042, CTONG2000063, CTONG2000201, CTONG2000218, CTONG2000279, CTONG2000303, CTONG2000330, CTONG2000395, CTONG2000411, CTONG2000452, CTONG2000465, CTONG2000480, CTONG2000508, CTONG2000561, CTONG2000589, CTONG2000766, CTONG2000771, CTONG2000819, CTONG2000827, CTONG2000846, CTONG2000855, CTONG2000977, CTONG2001139, CTONG2001320, CTONG2001366, CTONG2001413, CTONG2001428, CTONG2001513, CTONG2001524, CTONG2001533, CTONG2001598, CTONG2001681, CTONG2001724, CTONG2001748, CTONG2001749, CTONG2001800, CTONG2001820, CTONG2001858, CTONG2001877, CTONG2001911, CTONG2001932, CTONG2001955, CTONG2002066, CTONG2002073, CTONG2002095, CTONG2002143, CTONG2002270, CTONG2002395, CTONG2002417, CTONG2002418,

CTONG2002520, CTONG2002558, CTONG2002590, CTONG2002709, CTONG2002721, CTONG2002744, CTONG2002766, CTONG2002803, CTONG2002816, CTONG2002820, CTONG2002832, CTONG2002841, CTONG2002965, CTONG2002999, CTONG2003028, CTONG2003094, CTONG2003100, CTONG2003115, CTONG2003189, CTONG2003245, CTONG2003293, CTONG2003348, CTONG2003350, CTONG2003361, CTONG2003372, CTONG2003375, CTONG2003427, CTONG2003517, CTONG2003599, CTONG2003680, CTONG2003699, CTONG2003764, CTONG2003782, CTONG2003889, CTONG2003945, CTONG2004000, CTONG2004062, CTONG2004115, CTONG2004126, CTONG2004264, CTONG2004305, CTONG2004397, CTONG2004423, CTONG2004454, CTONG2004669, CTONG2004716, CTONG2004912, CTONG2004941, CTONG2004948, CTONG2005028,

CTONG2005049, CTONG2005110, CTONG2005145, CTONG2005278, CTONG2005290, CTONG2005374, CTONG2005399, CTONG2005468, CTONG2005567, CTONG2005585, CTONG2005615, CTONG2005635, CTONG2005775, CTONG2005795, CTONG2005890, CTONG2005904, CTONG2005913, CTONG2006129, CTONG2006223, CTONG2006235, CTONG2006273, CTONG2006310, CTONG2006377, CTONG2006393, CTONG2006412, CTONG2006449, CTONG2006515, CTONG2006524, CTONG2006568, CTONG2006620, CTONG2006666, CTONG2006691, CTONG2006709, CTONG2006798, CTONG2006836, CTONG2006942, CTONG2006964, CTONG2007007, CTONG2007009, CTONG2007091, CTONG2007104, CTONG2007168, CTONG2007175, CTONG2007259, CTONG2007272, CTONG2007293, CTONG2007297, CTONG2007399, CTONG2007400, CTONG2007417,

CTONG2007441, CTONG2007474, CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008321, CTONG2008343, CTONG2008402, CTONG2008405, CTONG2008466, CTONG2008518, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604,

CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010408, CTONG2010508, CTONG2010566, CTONG2010623, CTONG2010649, CTONG2010652, CTONG2010803, CTONG2011188, CTONG2011429, CTONG2011474, CTONG2011676, CTONG2011716, CTONG2011765, CTONG2011779, CTONG2011801, CTONG2011815, CTONG2011822, CTONG2011825, CTONG2011920, CTONG2011985, CTONG2012001, CTONG2012050, CTONG2012077, CTONG2012101, CTONG2012123, CTONG2012230, CTONG2012401, CTONG2012422,

CTONG2012425, CTONG2012447, CTONG2012453, CTONG2012473, CTONG2012476, CTONG2012533, CTONG2012554, CTONG2012564, CTONG2012661, CTONG2012724, CTONG2012738, CTONG2012745, CTONG2012758, CTONG2012843, CTONG2012879, CTONG2012901, CTONG2013128, CTONG2013149, CTONG2013156, CTONG2013178, CTONG2013222, CTONG2013284, CTONG2013339, CTONG2013352, CTONG2013381, CTONG2013393, CTONG2013511, CTONG2013630, CTONG2013803, CTONG2013934, CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014369, CTONG2014389, CTONG2014491, CTONG2014630, CTONG2014635, CTONG2014646, CTONG2014705, CTONG2014898, CTONG2014946,

CTONG2014954, CTONG2014959, CTONG2014966, CTONG2014995, CTONG2015204, CTONG2015316, CTONG2015330, CTONG2015345, CTONG2015358, CTONG2015434, CTONG2015540, CTONG2015596, CTONG2015633, CTONG2015678, CTONG2015717, CTONG2015804, CTONG2015815, CTONG2015866, CTONG2015914, CTONG2015953, CTONG2016185, CTONG2016217, CTONG2016355, CTONG2016408, CTONG2016499, CTONG2016505, CTONG2016559, CTONG2016658, CTONG2016775, CTONG2016797, CTONG2016846, CTONG2016904, CTONG2016942, CTONG2016953, CTONG2017021, CTONG2017094, CTONG2017292, CTONG2017409, CTONG2017458, CTONG2017500, CTONG2017604, CTONG2017650, CTONG2017798, CTONG2017804, CTONG2017814, CTONG2017910, CTONG2017939, CTONG2017989, CTONG2017998, CTONG2018062,

CTONG2018069, CTONG2018147, CTONG2018211, CTONG2018217, CTONG2018279, CTONG2018343, CTONG2018379, CTONG2018383, CTONG2018413, CTONG2018483, CTONG2018629, CTONG2018632, CTONG2018637, CTONG2018652, CTONG2018653, CTONG2018655, CTONG2018735, CTONG2018739, CTONG2018808, CTONG2018843, CTONG2018898, CTONG2018900, CTONG2018976, CTONG2019029, CTONG2019063, CTONG2019144, CTONG2019232, CTONG2019248, CTONG2019400, CTONG2019590, CTONG2019652, CTONG2019704, CTONG2019788, CTONG2019822, CTONG2019833, CTONG2020031, CTONG2020108, CTONG2020127, CTONG2020171, CTONG2020374, CTONG2020378, CTONG2020411, CTONG2020445, CTONG2020522, CTONG2020560, CTONG2020582, CTONG2020638, CTONG2020806, CTONG2020831, CTONG2020974,

CTONG2021024, CTONG2021132, CTONG2021168, CTONG2021289, CTONG2022153, CTONG2022601, CTONG2023021, CTONG2023449, CTONG2023512, CTONG2024031, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028208, CTONG2028309, CTONG2028486, CTONG2028518, CTONG2028687, CTONG2028758, CTONG3000657, CTONG3000686, CTONG3000707, CTONG3000896, CTONG3001123, CTONG3001420, CTONG3001501, CTONG3001560, CTONG3001605, CTONG3001746, CTONG3002020, CTONG3002127,

CTONG3002370, CTONG3002412, CTONG3002433, CTONG3002518, CTONG3002552, CTONG3002588, CTONG3002674, CTONG3002728, CTONG3002835, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258,

CTONG3008457, CTONG3008496, CTONG3008566, CTONG3008639, CTONG3008831, CTONG3008951, CTONG3008959, CTONG3009227, CTONG3009287, CTONG3009328, CTONG3009385, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001670, D3OST2001788, D3OST2002182, D3OST2002223, D3OST2002262, D3OST2002272, D3OST2002417, D3OST2002436, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000358, D6OST2000507, D9OST2000278, D9OST2000440, D9OST2000869, D9OST2001319,

D9OST2001433, D9OST2002397, D9OST2003106, D9OST2003108, D9OST2003137, D9OST2003397, D9OST2003580, D9OST2003594, D9OST2003762, D9OST2003791, D9OST2003989, D9OST2004018, D9OST2004073, D9OST2004417, DFNES1000003, DFNES1000083, DFNES1000107, DFNES1000185, DFNES2000143, DFNES2000146, DFNES2000212, DFNES2000292, DFNES2000335, DFNES2000426, DFNES2000432, DFNES2000443, DFNES2000457, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002550, DFNES2002588, DFNES2002817, DFNES2002966, DFNES2003081, DFNES2003192, DFNES2003255, DFNES2003742, DFNES2004346,

DFNES2004354, DFNES2004383, DFNES2004608, DFNES2004684, DFNES2005266, DFNES2005527, DFNES2005540, DFNES2005690, DFNES2005779, DFNES2005875, DFNES2006346, DFNES2006944, DFNES2007113, DFNES2007299, DFNES2007332, DFNES2007634, DFNES2007641, DFNES2007757, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000024, FCBBF1000025, FCBBF1000038, FCBBF1000053, FCBBF1000061, FCBBF1000063, FCBBF1000069, FCBBF1000077, FCBBF1000115, FCBBF1000117, FCBBF1000118, FCBBF1000125, FCBBF1000131, FCBBF1000139, FCBBF1000155, FCBBF1000157, FCBBF1000171, FCBBF1000174,

FCBBF1000197, FCBBF1000198, FCBBF1000206, FCBBF1000220, FCBBF1000232, FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000367, FCBBF1000374, FCBBF1000376, FCBBF1000377, FCBBF1000395, FCBBF1000397, FCBBF1000403, FCBBF1000412, FCBBF1000425, FCBBF1000437, FCBBF1000449, FCBBF1000476, FCBBF1000509, FCBBF1000546, FCBBF1000550, FCBBF1000556, FCBBF1000574, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF2000038, FCBBF2000087, FCBBF2000094, FCBBF2000105,

FCBBF2000123, FCBBF2000195, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566, FCBBF2000576, FCBBF2000591, FCBBF2000677, FCBBF2000678, FCBBF2000685, FCBBF2000733, FCBBF2000782, FCBBF2000808, FCBBF2000815, FCBBF2000825, FCBBF2000940, FCBBF2000951, FCBBF2001001, FCBBF2001116, FCBBF2001183, FCBBF2001188, FCBBF2001211, FCBBF2001238, FCBBF2001243, FCBBF2001291, FCBBF2001299, FCBBF2001309, FCBBF2001400, FCBBF2001427, FCBBF2001480, FCBBF2001529, FCBBF2001538, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002370,

FCBBF2002541, FCBBF2002626, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005909, FCBBF2006131, FCBBF2006380, FCBBF2006418, FCBBF2006452, FCBBF2006634, FCBBF2006781, FCBBF2007012, FCBBF2007186, FCBBF2007265, FCBBF2007302,

FCBBF2007556, FCBBF2007610, FCBBF2007633, FCBBF2007840, FCBBF3000102, FCBBF3000120, FCBBF3000168, FCBBF3000175, FCBBF3000227, FCBBF3000228, FCBBF3000229, FCBBF3000233, FCBBF3000269, FCBBF3000361, FCBBF3000367, FCBBF3000388, FCBBF3000462, FCBBF3000473, FCBBF3000501, FCBBF3000518, FCBBF3000550, FCBBF3000847, FCBBF3001018, FCBBF3001020, FCBBF3001081, FCBBF3001235, FCBBF3001281, FCBBF3001302, FCBBF3001377, FCBBF3001594, FCBBF3001657, FCBBF3001818, FCBBF3001855, FCBBF3001890, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925,

FCBBF3003216, FCBBF3003305, FCBBF3003373, FCBBF3003475, FCBBF3003481, FCBBF3003557, FCBBF3003787, FCBBF3003902, FCBBF3004021, FCBBF3004041, FCBBF3004261, FCBBF3004323, FCBBF3004390, FCBBF3004409, FCBBF3004502, FCBBF3004715, FCBBF3004842, FCBBF3004847, FCBBF3004955, FCBBF3005160, FCBBF3005218, FCBBF3005444, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007631, FCBBF3007855, FCBBF3007859, FCBBF3007860, FCBBF3008073, FCBBF3008100, FCBBF3008153, FCBBF3008159, FCBBF3008251,

FCBBF3008311, FCBBF3008362, FCBBF3008382, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009541, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889,

FCBBF3012170, FCBBF3012182, FCBBF3012288, FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016178, FCBBF3016451, FCBBF3016618, FCBBF3016622, FCBBF3016644, FCBBF3016667, FCBBF3016928, FCBBF3016987, FCBBF3017024, FCBBF3017059, FCBBF3017071, FCBBF3017123, FCBBF3017233, FCBBF3017255, FCBBF3017288,

FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019716, FCBBF3019784, FCBBF3019839, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894,

FCBBF3023061, FCBBF3023078, FCBBF3023368, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202,

FCBBF3028472, FCBBF3028528, FCBBF3028582, FCBBF3028593, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000500, FCBBF4000529, FCBBF4000548, FCBBF5000041, FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000022, FEBRA1000030, FEBRA1000057, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000220, FEBRA2000253, FEBRA2000282, FEBRA2000286,

FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000452, FEBRA2000454, FEBRA2000462, FEBRA2000468, FEBRA2000491, FEBRA2000504, FEBRA2000532, FEBRA2000538, FEBRA2000575, FEBRA2000581, FEBRA2000656, FEBRA2000659, FEBRA2000680, FEBRA2000690, FEBRA2000727, FEBRA2000734, FEBRA2000740, FEBRA2000757, FEBRA2000762, FEBRA2000771, FEBRA2000772, FEBRA2000782, FEBRA2000787, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000909,

FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001245, FEBRA2001267, FEBRA2001294, FEBRA2001334, FEBRA2001351, FEBRA2001438, FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001752, FEBRA2001772, FEBRA2001790, FEBRA2001828, FEBRA2001867, FEBRA2001914, FEBRA2001974,

FEBRA2001989, FEBRA2001990, FEBRA2002009, FEBRA2002086, FEBRA2002109, FEBRA2002191, FEBRA2002260, FEBRA2002352, FEBRA2002399, FEBRA2002410, FEBRA2002429, FEBRA2002442, FEBRA2002456, FEBRA2002508, FEBRA2002525, FEBRA2002527, FEBRA2002552, FEBRA2002574, FEBRA2002601, FEBRA2002611, FEBRA2002628, FEBRA2002662, FEBRA2002682, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002933, FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436,

FEBRA20.03454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003750, FEBRA2003822, FEBRA2003833, FEBRA2003897, FEBRA2003907, FEBRA2004023, FEBRA2004026, FEBRA2004029, FEBRA2004042, FEBRA2004053, FEBRA2004056, FEBRA2004091, FEBRA2004110, FEBRA2004191, FEBRA2004202, FEBRA2004219, FEBRA2004237, FEBRA2004268, FEBRA2004293, FEBRA2004329, FEBRA2004331, FEBRA2004443, FEBRA2004485, FEBRA2004490, FEBRA2004538, FEBRA2004592, FEBRA2004620, FEBRA2004628, FEBRA2004651, FEBRA2004767, FEBRA2004813, FEBRA2004840, FEBRA2004852, FEBRA2004867, FEBRA2004914, FEBRA2004933, FEBRA2004987, FEBRA2005014, FEBRA2005036, FEBRA2005079,

FEBRA2005114, FEBRA2005216, FEBRA2005291, FEBRA2005361, FEBRA2005377, FEBRA2005380, FEBRA2005416, FEBRA2005427, FEBRA2005458, FEBRA2005476, FEBRA2005572, FEBRA2005701, FEBRA2005726, FEBRA2005734, FEBRA2005787, FEBRA2005788, FEBRA2005824, FEBRA2005995, FEBRA2005998, FEBRA2006061, FEBRA2006112, FEBRA2006150, FEBRA2006165, FEBRA2006255, FEBRA2006270, FEBRA2006315, FEBRA2006346, FEBRA2006354, FEBRA2006357, FEBRA2006368, FEBRA2006379, FEBRA2006396, FEBRA2006401, FEBRA2006409, FEBRA2006418, FEBRA2006427, FEBRA2006476, FEBRA2006491, FEBRA2006519, FEBRA2006521, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890,

FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007620, FEBRA2007622, FEBRA2007708, FEBRA2007714, FEBRA2007793, FEBRA2007818, FEBRA2007823, FEBRA2007900, FEBRA2007901, FEBRA2007931, FEBRA2007957, FEBRA2008009, FEBRA2008086, FEBRA2008134, FEBRA2008135, FEBRA2008159, FEBRA2008165, FEBRA2008172, FEBRA2008201, FEBRA2008210, FEBRA2008265, FEBRA2008266, FEBRA2008282, FEBRA2008287, FEBRA2008311, FEBRA2008341, FEBRA2008360, FEBRA2008459, FEBRA2008468, FEBRA2008475, FEBRA2008481, FEBRA2008542,

FEBRA2008583, FEBRA2008660, FEBRA2008662, FEBRA2008681, FEBRA2008692, FEBRA2008741, FEBRA2008755, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009419, FEBRA2009437, FEBRA2009478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746,

FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200,

FEBRA2017223, FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022148, FEBRA2022204, FEBRA2022255,

FEBRA2022322, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023377, FEBRA2023399, FEBRA2023498, FEBRA2023550, FEBRA2023721, FEBRA2023927, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025427, FEBRA2025463, FEBRA2025477, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366,

FEBRA2028457, FEBRA2028477, FEBRA2028503, FEHRT2000325, FEHRT2000364, FEKID1000018, FELIV1000101, FELIV1000137, FELIV1000164, FELNG2000241, HCASM1000021, HCASM1000050, HCASM1000061, HCASM1000115, HCASM1000130, HCASM2000138, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154,

HCASM2008536, HCASM2009405, HCASM2009424, HCASM2009463, HCASM2009588, HCHON1000015, HCHON1000131, HCHON1000142, HCHON1000166, HCHON1000176, HCHON2000028, HCHON2000038, HCHON2000062, HCHON2000100, HCHON2000156, HCHON2000199, HCHON2000212, HCHON2000271, HCHON2000295, HCHON2000315, HCHON2000322, HCHON2000323, HCHON2000344, HCHON2000373, HCHON2000395, HCHON2000417, HCHON2000418, HCHON2000473, HCHON2000503, HCHON2000660, HCHON2000663, HCHON2000673, HCHON2000676, HCHON2000698, HCHON2000699, HCHON2000705, HCHON2000743, HCHON2000751, HCHON2000815, HCHON2000817, HCHON2000818, HCHON2000819, HCHON2000832, HCHON2000851, HCHON2000935, HCHON2000942, HCHON2000956, HCHON2000994, HCHON2001084, HCHON2001099,

HCHON2001110, HCHON2001116, HCHON2001161, HCHON2001200, HCHON2001214, HCHON2001217, HCHON2001233, HCHON2001269, HCHON2001450, HCHON2001453, HCHON2001497, HCHON2001519, HCHON2001523, HCHON2001535, HCHON2001548, HCHON2001577, HCHON2001598, HCHON2001604, HCHON2001607, HCHON2001646, HCHON2001712, HCHON2001768, HCHON2001801, HCHON2001853, HCHON2002123, HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118,

HCHON2005166, HCHON2005802, HCHON2005921, HCHON2005987, HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HCHON2010543, HEART1000010, HEART1000088, HEART1000099, HEART1000102, HEART1000118, HEART1000132, HEART1000139, HEART1000142, HEART1000151, HEART1000173, HEART2000024, HEART2000035, HEART2000099, HEART2000298, HEART2000306, HEART2000309, HEART2000411,

HEART2000418, HEART2000444, HEART2000448, HEART2000492, HEART2000506, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003781, HEART2003836, HEART2003852, HEART2004570, HEART2004764, HEART2004931, HEART2004940, HEART2004980, HEART2005244, HEART2005310, HEART2005581, HEART2005610, HEART2006131, HEART2006195, HEART2006310, HEART2006334, HEART2006487, HEART2006521, HEART2006557, HEART2006787, HEART2006789, HEART2006909, HEART2007031, HEART2007231, HEART2007443, HEART2007696, HEART2007767, HEART2008108,

HEART2008111, HEART2008257, HEART2008509, HEART2008994, HEART2009000, HEART2009599, HEART2009680, HEART2010391, HEART2010492, HEART2010495, LIVER1000017, LIVER1000050, LIVER1000055, LIVER1000058, LIVER1000063, LIVER1000067, LIVER1000073, LIVER1000082, LIVER1000099, LIVER1000111, LIVER1000126, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000033, LIVER2000037, LIVER2000158, LIVER2000216, LIVER2000237, LIVER2000247, LIVER2000360, LIVER2000416, LIVER2000446, LIVER2000515, LIVER2000592, LIVER2000626, LIVER2000682, LIVER2000769,

LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2001608, LIVER2001835, LIVER2002638, LIVER2002644, LIVER2002660, LIVER2002702, LIVER2003008, LIVER2003065, LIVER2003234, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006251, LIVER2006410, LIVER2006469, LIVER2006644, LIVER2006764, LIVER2007415,

LIVER2007548, LIVER2007568, LIVER2007721, LIVER2007783, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000096, MAMGL1000100, MAMGL1000132, MAMGL1000151, MAMGL1000178, MAMGL1000182, MAMGL1000184, MESAN1000032, MESAN1000035, MESAN1000050, MESAN1000079, MESAN1000080, MESAN1000101, MESAN1000121, MESAN1000126, MESAN1000147, MESAN1000180, MESAN2000023, MESAN2000026, MESAN2000067, MESAN2000092, MESAN2000149, MESAN2000167, MESAN2000291, MESAN2000434, MESAN2000457, MESAN2000462, MESAN2000501, MESAN2000620, MESAN2000711,

MESAN2000815, MESAN2000894, MESAN2000909, MESAN2001154, MESAN2001450, MESAN2001627, MESAN2001770, MESAN2001979, MESAN2002113, MESAN2002122, MESAN2002147, MESAN2002186, MESAN2002424, MESAN2002687, MESAN2002709, MESAN2002790, MESAN2002844, MESAN2002940, MESAN2002978, MESAN2003035, MESAN2003037, MESAN2003039, MESAN2003058, MESAN2003101, MESAN2003190, MESAN2003322, MESAN2003444, MESAN2003490, MESAN2003529, MESAN2003646, MESAN2003662, MESAN2003709, MESAN2003851, MESAN2004138, MESAN2004288, MESAN2004575, MESAN2005303, MESAN2005371, MESAN2005633, MESAN2005724, MESAN2005808, MESAN2005811, MESAN2005957, MESAN2005958, MESAN2006022, MESAN2006043, MESAN2006328, MESAN2006401, MESAN2006580, MESAN2006599,

MESAN2006743, MESAN2006953, MESAN2007032, MESAN2007440, MESAN2007597, MESAN2007755, MESAN2008222, MESAN2008334, MESAN2008415, MESAN2008460, MESAN2008536, MESAN2008729, MESAN2008821, MESAN2008926, MESAN2008936, MESAN2009019, MESAN2009081, MESAN2009105, MESAN2009311, MESAN2009418, MESAN2009503, MESAN2009522, MESAN2009580, MESAN2010031, MESAN2010114, MESAN2010312, MESAN2010321, MESAN2010664, MESAN2010681, MESAN2011545, MESAN2011597, MESAN2011627, MESAN2011632, MESAN2011977, MESAN2011984, MESAN2012054, MESAN2012113, MESAN2012586, MESAN2012708, MESAN2012735, MESAN2013019, MESAN2013022, MESAN2013183, MESAN2013211, MESAN2013283, MESAN2013284, MESAN2013611, MESAN2013674, MESAN2013908, MESAN2013936,

MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2016159, MESAN2016304, MESAN2016409, MESAN2016552, MESAN2016965, MESAN2017133, MESAN2017152, MESAN2017373, MESAN2017417, MESAN2018209, MESAN2018576, MESAN2018670, MESAN2018699, MESTC1000042, MESTC1000147, MESTC2000150, MESTC2000153, MESTC2000170, N1ESE2000698, NB9N41000011, NB9N41000047, NB9N41000121, NB9N41000135, NB9N41000142, NB9N41000146, NB9N41000340, NB9N42000104, NB9N42000122, NB9N42000196, NB9N42000281, NB9N42000314, NB9N42000342, NB9N42000430, NB9N42000495, NB9N42001300, NCRRP1000129, NESOP1000010, NESOP1000087, NESOP1000108,

NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000102, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000014, NT2NE1000018, NT2NE1000023,

NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000056, NT2NE2000057, NT2NE2000064, NT2NE2000109, NT2NE2000137, NT2NE2000156, NT2NE2000222, NT2NE2000259, NT2NE2000284, NT2NE2000299, NT2NE2000327, NT2NE2000353, NT2NE2000356, NT2NE2000369, NT2NE2000374, NT2NE2000383, NT2NE2000384, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000809, NT2NE2000864, NT2NE2000909,

NT2NE2000913, NT2NE2000950, NT2NE2000951, NT2NE2000980, NT2NE2001005, NT2NE2001021, NT2NE2001040, NT2NE2001049, NT2NE2001142, NT2NE2001156, NT2NE2001180, NT2NE2001181, NT2NE2001188, NT2NE2001191, NT2NE2001247, NT2NE2001252, NT2NE2001324, NT2NE2001326, NT2NE2001337, NT2NE2001364, NT2NE2001372, NT2NE2001397, NT2NE2001403, NT2NE2001428, NT2NE2001432, NT2NE2001442, NT2NE2001524, NT2NE2001545, NT2NE2001598, NT2NE2001617, NT2NE2001623, NT2NE2001626, NT2NE2001634, NT2NE2001648, NT2NE2001660, NT2NE2001666, NT2NE2001677, NT2NE2001697, NT2NE2001698, NT2NE2001723, NT2NE2001725, NT2NE2001793, NT2NE2001874, NT2NE2001889, NT2NE2001923, NT2NE2002100, -NT2NE2002162, NT2NE2002186, NT2NE2002285, NT2NE2002311,

NT2NE2002620, NT2NE2002651, NT2NE2002732, NT2NE2002768, NT2NE2002791, NT2NE2002856, NT2NE2003150, NT2NE2003185, NT2NE2003252, NT2NE2003280, NT2NE2003308, NT2NE2003309, NT2NE2003315, NT2NE2003408, NT2NE2003457, NT2NE2003485, NT2NE2003540, NT2NE2003569, NT2NE2003571, NT2NE2003705, NT2NE2003752, NT2NE2003887, NT2NE2003921, NT2NE2004255, NT2NE2004378, NT2NE2004490, NT2NE2004606, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005419, NT2NE2005441, NT2NE2005517, NT2NE2005537, NT2NE2005555, NT2NE2005588, NT2NE2005676, NT2NE2005688, NT2NE2005720, NT2NE2005784, NT2NE2005804, NT2NE2005821,

NT2NE2005876, NT2NE2005890, NT2NE2005921, NT2NE2005968, NT2NE2006075, NT2NE2006087, NT2NE2006217, NT2NE2006288, NT2NE2006382, NT2NE2006387, NT2NE2006427, NT2NE2006432, NT2NE2006458, NT2NE2006478, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608,

NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010854, NT2NE2011033, NT2NE2011036, NT2NE2011107, NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012847, NT2NE2012928, NT2NE2013019, NT2NE2013081, NT2NE2013189, NT2NE2013217, NT2NE2013547, NT2NE2014013, NT2NE2014028, NT2NE2014113, NT2NE2014176, NT2NE2014221,

NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018273, NT2NE2018376, NT2NE2018472, NT2NE2018490, NT2NE2018594, NT2NE2018739, NT2NE2018916,

NT2NE2019056, NT2NE2019091, NT2NE2019099, NT2RI1000016, NT2RI1000035, NT2RI1000048, NT2RI1000164, NT2RI1000172, NT2RI2000004, NT2RI2000007, NT2RI2000116, NT2RI2000117, NT2RI2000128, NT2RI2000133, NT2RI2000167, NT2RI2000173, NT2RI2000187, NT2RI2000255, NT2RI2000270, NT2RI2000276, NT2RI2000294, NT2RI2000313, NT2RI2000341, NT2RI2000344, NT2RI2000348, NT2RI2000412, NT2RI2000421, NT2RI2000480, NT2RI2000575, NT2RI2000588, NT2RI2000597, NT2RI2000669, NT2RI2000688, NT2RI2000689, NT2RI2000704, NT2RI2000738, NT2RI2000775, NT2RI2000822, NT2RI2000829, NT2RI2000841, NT2RI2000865, NT2RI2000932, NT2RI2000987, NT2RI2001010, NT2RI2001017, NT2RI2001030, NT2RI2001083, NT2RI2001091, NT2RI2001167, NT2RI2001178,

NT2RI2001230, NT2RI2001235, NT2RI2001244, NT2RI2001307, NT2RI2001342, NT2RI2001409, NT2RI2001410, NT2RI2001422, NT2RI2001425, NT2RI2001449, NT2RI2001450, NT2RI2001474, NT2RI2001519, NT2RI2001595, NT2RI2001621, NT2RI2001624, NT2RI2001726, NT2RI2001769, NT2RI2001782, NT2RI2001836, NT2RI2001866, NT2RI2001952, NT2RI2002022, NT2RI2002041, NT2RI2002117, NT2RI2002120, NT2RI2002153, NT2RI2002179, NT2RI2002243, NT2RI2002252, NT2RI2002260, NT2RI2002270, NT2RI2002334, NT2RI2002359, NT2RI2002447, NT2RI2002481, NT2RI2002507, NT2RI2002517, NT2RI2002530, NT2RI2002540, NT2RI2002549, NT2RI2002564, NT2RI2002585, NT2RI2002592, NT2RI2002602, NT2RI2002654, NT2RI2002699, NT2RI2002802, NT2RI2002819, NT2RI2002822,

NT2RI2002847, NT2RI2002852, NT2RI2002865, NT2RI2002923, NT2RI2002926, NT2RI2002958, NT2RI2002970, NT2RI2003011, NT2RI2003019, NT2RI2003051, NT2RI2003067, NT2RI2003205, NT2RI2003222, NT2RI2003301, NT2RI2003338, NT2RI2003360, NT2RI2003361, NT2RI2003383, NT2RI2003392, NT2RI2003399, NT2RI2003407, NT2RI2003420, NT2RI2003481, NT2RI2003487, NT2RI2003556, NT2RI2003655, NT2RI2003667, NT2RI2003678, NT2RI2003751, NT2RI2003884, NT2RI2003973, NT2RI2004059, NT2RI2004078, NT2RI2004079, NT2RI2004093, NT2RI2004136, NT2RI2004157, NT2RI2004188, NT2RI2004190, NT2RI2004284, NT2RI2004290, NT2RI2004304, NT2RI2004381, NT2RI2004398, NT2RI2004410, NT2RI2004442, NT2RI2004468, NT2RI2004606, NT2RI2004618, NT2RI2004783,

NT2RI2004818, NT2RI2004840, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005116, NT2RI2005130, NT2RI2005166, NT2RI2005239, NT2RI2005246, NT2RI2005315, NT2RI2005353, NT2RI2005358, NT2RI2005382, NT2RI2005473, NT2RI2005520, NT2RI2005564, NT2RI2005579, NT2RI2005621, NT2RI2005628, NT2RI2005647, NT2RI2005670, NT2RI2005710, NT2RI2005772, NT2RI2005811, NT2RI2005816, NT2RI2005851, NT2RI2005966, NT2RI2006071, NT2RI2006072, NT2RI2006081, NT2RI2006126, NT2RI2006127, NT2RI2006183, NT2RI2006257, NT2RI2006271, NT2RI2006294, NT2RI2006345, NT2RI2006403, NT2RI2006445, NT2RI2006487, NT2RI2006506, NT2RI2006640,

NT2RI2006647, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006825, NT2RI2006838, NT2RI2006855, NT2RI2006943, NT2RI2006973, NT2RI2007046, NT2RI2007048, NT2RI2007096, NT2RI2007116, NT2RI2007148, NT2RI2007214, NT2RI2007277, NT2RI2007303, NT2RI2007334, NT2RI2007384, NT2RI2007386, NT2RI2007406, NT2RI2007439, NT2RI2007445, NT2RI2007468, NT2RI2007469, NT2RI2007507, NT2RI2007589, NT2RI2007723, NT2RI2007729, NT2RI2007751, NT2RI2007754, NT2RI2007827, NT2RI2007853, NT2RI2007877, NT2RI2007879, NT2RI2007891, NT2RI2007956, NT2RI2007965, NT2RI2008007, NT2RI2008050, NT2RI2008141, NT2RI2008171,

NT2RI2008188, NT2RI2008204, NT2RI2008221, NT2RI2008233, NT2RI2008455, NT2RI2008502, NT2RI2008566, NT2RI2008598, NT2RI2008622, NT2RI2008656, NT2RI2008714, NT2RI2008724, NT2RI2008791, NT2RI2008801, NT2RI2008808, NT2RI2008812, NT2RI2008841, NT2RI200.8942, NT2RI2008952, NT2RI2009005, NT2RI2009083, NT2RI2009101, NT2RI2009103, NT2RI2009144, NT2RI2009151, NT2RI2009215, NT2RI2009233, NT2RI2009259, NT2RI2009269, NT2RI2009281, NT2RI2009289, NT2RI2009301, NT2RI2009402, NT2RI2009517, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012659, NT2RI2012726, NT2RI2012990, NT2RI2013345, NT2RI2013357, NT2RI2013373,

NT2RI2014247, NT2RI2014551, NT2RI2014683, NT2RI2014733, NT2RI2015239, NT2RI2015533, NT2RI2016128, NT2RI2016134, NT2RI2017529, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213,

NT2RI3000423, NT2RI3000622, NT2RI3001132, NT2RI3001263, NT2RI3001279, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006284, NT2RI3006340, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443, NT2RI3007684, NT2RI3007757,

NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000008, NT2RP6000017, NT2RP6000032, NT2RP6000033, NT2RP6000039, NT2RP6000043, NT2RP6000059, NT2RP6000060, NT2RP6000077, NT2RP6000085, NT2RP6000086, NT2RP6000100, NT2RP6000109, NT2RP6000110, NT2RP6000119, NT2RP6000127, NT2RP6000130, NT2RP6000140, NT2RP6000150, NT2RP7000041, NT2RP7000069, NT2RP7000086, NT2RP7000109, NT2RP7000112, NT2RP7000238, NT2RP7000259, NT2RP7000311, NT2RP7000359, NT2RP7000364, NT2RP7000380, NT2RP7000391, NT2RP7000425, NT2RP7000579, NT2RP7000586, NT2RP7000624, NT2RP7000812, NT2RP7000926,

NT2RP7001074, NT2RP7001080, NT2RP7001156, NT2RP7001231, NT2RP7001306, NT2RP7001319, NT2RP7001335, NT2RP7001364, NT2RP7001475, NT2RP7001532, NT2RP7001591, NT2RP7001602, NT2RP7001678, NT2RP7001856, NT2RP7002028, NT2RP7002064, NT2RP7002151, NT2RP7002163, NT2RP7002243, NT2RP7002282, NT2RP7002376, NT2RP7002379, NT2RP7002450, NT2RP7002478, NT2RP7002554, NT2RP7002594, NT2RP7002603, NT2RP7002619, NT2RP7002650, NT2RP7002738, NT2RP7002779, NT2RP7002802, NT2RP7002841, NT2RP7002875, NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003203, NT2RP7003261, NT2RP7003319, NT2RP7003439, NT2RP7003511,

NT2RP7003629, NT2RP7003632, NT2RP7003647, NT2RP7003680, NT2RP7003724, NT2RP7003960, NT2RP7004027, NT2RP7004080, NT2RP7004114, NT2RP7004123, NT2RP7004173, NT2RP7004196, NT2RP7004204, NT2RP7004233, NT2RP7004260, NT2RP7004352, NT2RP7004358, NT2RP7004396, NT2RP7004481, NT2RP7004541, NT2RP7004559, NT2RP7004641, NT2RP7004656, NT2RP7004687, NT2RP7004722, NT2RP7004766, NT2RP7004790, NT2RP7004915, NT2RP7004925, NT2RP7004961, NT2RP7004964, NT2RP7004975, NT2RP7005118, NT2RP7005205, NT2RP7005219, NT2RP7005323, NT2RP7005468, NT2RP7005493, NT2RP7005513, NT2RP7005529, NT2RP7005629, NT2RP7005631, NT2RP7005669, NT2RP7005675, NT2RP7005750, NT2RP7005846, NT2RP7006033, NT2RP7006075, NT2RP7006160, NT2RP7006162,

NT2RP7006188, NT2RP7006223, NT2RP7006263, NT2RP7006296, NT2RP7006304, NT2RP7006374, NT2RP7006395, NT2RP7006408, NT2RP7006457, NT2RP7006490, NT2RP7006539, NT2RP7006547, NT2RP7006601, NT2RP7006619, NT2RP7006621, NT2RP7006636, NT2RP7006701, NT2RP7006853, NT2RP7006887, NT2RP7006980, NT2RP7006995, NT2RP7007114, NT2RP7007221, NT2RP7007226, NT2RP7007252, NT2RP7007269, NT2RP7007310, NT2RP7007359, NT2RP7007381, NT2RP7007406, NT2RP7007480, NT2RP7007504, NT2RP7007530, NT2RP7007537, NT2RP7007580, NT2RP7007594, NT2RP7007617, NT2RP7007643, NT2RP7007766, NT2RP7007842, NT2RP7007925, NT2RP7007930, NT2RP7007975, NT2RP7008013, NT2RP7008133, NT2RP7008137, NT2RP7008142, NT2RP7008144, NT2RP7008161, NT2RP7008167,

NT2RP7008315, NT2RP7008360, NT2RP7008396, NT2RP7008406, NT2RP7008435, NT2RP7008441, NT2RP7008454, NT2RP7008487, NT2RP7008543, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009087, NT2RP7009097, NT2RP7009108, NT2RP7009147, NT2RP7009149, NT2RP7009168, NT2RP7009215, NT2RP7009225, NT2RP7009236, NT2RP7009253, NT2RP7009259, NT2RP7009267, NT2RP7009322, NT2RP7009363, NT2RP7009370, NT2RP7009373, NT2RP7009394, NT2RP7009397, NT2RP7009429, NT2RP7009439, NT2RP7009466, NT2RP7009481, NT2RP7009482, NT2RP7009498, NT2RP7009502, NT2RP7009507, NT2RP7009577, NT2RP7009743, NT2RP7009867,

NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365,

NT2RP7017474, NT2RP7017546, NT2RP7017567, NT2RP7017795, NT2RP7017971, NT2RP7018032, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, NT2RP7019835, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000033,

NTONG1000047, NTONG1000052, NTONG1000075, NTONG1000087, NTONG1000123, NTONG1000130, NTONG1000161, NTONG1000172, NTONG1000182, NTONG1000213, NTONG1000214, NTONG1000247, NTONG1000264, NTONG2000040, NTONG2000107, NTONG2000231, NTONG2000265, NTONG2000334, NTONG2000363, NTONG2000413, NTONG2000492, NTONG2000499, NTONG2000531, NTONG2000583, NTONG2000858, NTONG2000876, NTONG2000878, NTONG2000966, NTONG2000977, NTONG2000985, NTONG2001014, NTONG2001079, NTONG2001157, NTONG2001173, NTONG2001220, NTONG2001222, NTONG2001362, NTONG2001428, NTONG2001532, NTONG2001550, NTONG2001587, NTONG2001619, NTONG2001642, NTONG2001657, NTONG2001762, NTONG2002278, NTONG2002582, NTONG2002807, NTONG2002948, NTONG2002970,

NTONG2002985, NTONG2003158, NTONG2003210, NTONG2003316, NTONG2003409, NTONG2003454, NTONG2003515, NTONG2003805, NTONG2003852, NTONG2003897, NTONG2003928, NTONG2004095, NTONG2004308, NTONG2004521, NTONG2004614, NTONG2004690, NTONG2004806, NTONG2004829, NTONG2004844, NTONG2004991, NTONG2005062, NTONG2005086, NTONG2005137, NTONG2005153, NTONG2005265, NTONG2005277, NTONG2005363, NTONG2005373, NTONG2005391, NTONG2005480, NTONG2005497, NTONG2005577, NTONG2005657, NTONG2005801, NTONG2005822, NTONG2005897, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028,

NTONG2007034, NTONG2007249, NTONG2007517, NTONG2007522, NTONG2007693, NTONG2007756, NTONG2008088, NTONG2008093, NTONG2008143, NTONG2008365, NTONG2008522, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030, OCBBF1000042, OCBBF1000049, OCBBF1000054, OCBBF1000067, OCBBF1000080, OCBBF1000085, OCBBF1000091, OCBBF1000104, OCBBF1000118, OCBBF1000119, OCBBF1000122, OCBBF1000130, OCBBF1000138, OCBBF1000145, OCBBF1000146, OCBBF1000153, OCBBF1000175, OCBBF1000185, OCBBF1000192, OCBBF1000254, OCBBF1000315, OCBBF2000013, OCBBF2000015, OCBBF2000126, OCBBF2000178, OCBBF2000231, OCBBF2000277,

OCBBF2000287, OCBBF2000323, OCBBF2000452, OCBBF2000677, OCBBF2000703, OCBBF2000712, OCBBF2000719, OCBBF2000831, OCBBF2000904, OCBBF2000982, OCBBF2000998, OCBBF2001075, OCBBF2001101, OCBBF2001124, OCBBF2001140, OCBBF2001166, OCBBF2001176, OCBBF2001186, OCBBF2001196, OCBBF2001210, OCBBF2001252, OCBBF2001307, OCBBF2001323, OCBBF2001389, OCBBF2001402, OCBBF2001408, OCBBF2001416, OCBBF2001473, OCBBF2001492, OCBBF2001527, OCBBF2001528, OCBBF2001639, OCBBF2001681, OCBBF2001706, OCBBF2001749, OCBBF2001794, OCBBF2001910, OCBBF2001931, OCBBF2001961, OCBBF2001983, OCBBF2002015, OCBBF2002086, OCBBF2002124, OCBBF2002161, OCBBF2002290, OCBBF2002357, OCBBF2002376, OCBBF2002615, OCBBF2002626, OCBBF2002656,

OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003518, OCBBF2003543, OCBBF2003593, OCBBF2003744, OCBBF2003819, OCBBF2003925, OCBBF2004038, OCBBF2004104, OCBBF2004147, OCBBF2004168, OCBBF2004235, OCBBF2004259, OCBBF2004273, OCBBF2004385, OCBBF2004418, OCBBF2004478, OCBBF2004482, OCBBF2004533, OCBBF2004567, OCBBF2004612, OCBBF2004647, OCBBF2004669, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843,

OCBBF2005901, OCBBF2005956, OCBBF2006005, OCBBF2006030, OCBBF2006058, OCBBF2006113, OCBBF2006151, OCBBF2006154, OCBBF2006214, OCBBF2006241, OCBBF2006313, OCBBF2006388, OCBBF2006438, OCBBF2006567, OCBBF2006624, OCBBF2006639, OCBBF2006660, OCBBF2006764, OCBBF2006849, OCBBF2006859, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007354, OCBBF2007414, OCBBF2007415, OCBBF2007428, OCBBF2007478, OCBBF2007485, OCBBF2007520, OCBBF2007555, OCBBF2007610, OCBBF2007622, OCBBF2007756, OCBBF2007762, OCBBF2007892, OCBBF2007931, OCBBF2007946, OCBBF2008005, OCBBF2008116,

OCBBF2008138, OCBBF2008144, OCBBF2008283, OCBBF2008330, OCBBF2008340, OCBBF2008366, OCBBF2008466, OCBBF2008483, OCBBF2008511, OCBBF2008520, OCBBF2008586, OCBBF2008640, OCBBF2008691, OCBBF2008701, OCBBF2008724, OCBBF2008768, OCBBF2008770, OCBBF2008775, OCBBF2008790, OCBBF2008814, OCBBF2008822, OCBBF2009095, OCBBF2009115, OCBBF2009242, OCBBF2009301, OCBBF2009352, OCBBF2009391, OCBBF2009424, OCBBF2009536, OCBBF2009571, OCBBF2009583, OCBBF2009603, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010841, OCBBF2010843,

OCBBF2010858, OCBBF2010863, OCBBF2010871, OCBBF2010931, OCBBF2010945, OCBBF2010978, OCBBF2010989, OCBBF2011021, OCBBF2011073, OCBBF2011137, OCBBF2011177, OCBBF2011228, OCBBF2011232, OCBBF2011283, OCBBF2011311, OCBBF2011625, OCBBF2011669, OCBBF2011685, OCBBF2011706, OCBBF2011722, OCBBF2011759, OCBBF2011767, OCBBF2011855, OCBBF2011872, OCBBF2011887, OCBBF2011897, OCBBF2011914, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012678, OCBBF2012704, OCBBF2012714, OCBBF2012755, OCBBF2012812, OCBBF2012821, OCBBF2012881, OCBBF2012908, OCBBF2012936, OCBBF2012990, OCBBF2013011,

OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789, OCBBF2013883, OCBBF2013926, OCBBF2014052, OCBBF2014089, OCBBF2014322, OCBBF2014386, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612,

OCBBF2016689, OCBBF2016690, OCBBF2016729, OCBBF2016841, OCBBF2016928, OCBBF2017055, OCBBF2017069, OCBBF2017306, OCBBF2017325, OCBBF2017398, OCBBF2017458, OCBBF2017489, OCBBF2017516, OCBBF2017665, OCBBF2017754, OCBBF2017791, OCBBF2017815, OCBBF2017882, OCBBF2017888, OCBBF2017920, OCBBF2018012, OCBBF2018014, OCBBF2018084, OCBBF2018167, OCBBF2018206, OCBBF2018229, OCBBF2018234, OCBBF2018362, OCBBF2018380, OCBBF2018563, OCBBF2018581, OCBBF2018618, OCBBF2018663, OCBBF2018687, OCBBF2018827, OCBBF2018828, OCBBF2018873, OCBBF2018934, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741,

OCBBF2020801, OCBBF2020825, OCBBF2020838, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173,

OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598,

OCBBF2037638, OCBBF2038238, OCBBF2038285, OCBBF2038317, OCBBF3000097, OCBBF3000213, OCBBF3000296, OCBBF3000372, OCBBF3000380, OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066,

PEBLM1000068, PEBLM1000071, PEBLM1000083, PEBLM1000144, PEBLM1000147, PEBLM1000180, PEBLM2000030, PEBLM2000112, PEBLM2000126, PEBLM2000170, PEBLM2000180, PEBLM2000213, PEBLM2000248, PEBLM2000270, PEBLM2000321, PEBLM2000326, PEBLM2000333, PEBLM2000338, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004452, PEBLM2004497, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215,

PEBLM2006283, PEBLM2006298, PEBLM2006366, PEBLM2006709, PEBLM2006912, PEBLM2007112, PEBLM2007140, PEBLM2007188, PEBLM2007296, PEBLM2007437, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957,

PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007068, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000066, PLACE5000067, PLACE5000068, PLACE5000070, PLACE5000080, PLACE5000105, PLACE5000113, PLACE5000115, PLACE5000116, PLACE5000129, PLACE5000139, PLACE5000153, PLACE5000159, PLACE5000170, PLACE5000171, PLACE5000178, PLACE5000245, PLACE5000260,

PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000145, PLACE6000191, PLACE6000205, PLACE6000231, PLACE6000263, PLACE6000296, PLACE6000325, PLACE6000348, PLACE6000371, PLACE6000414, PLACE6000424, PLACE6000426, PLACE6000429, PLACE6000450, PLACE6000523, PLACE6000550, PLACE6000552, PLACE6000555, PLACE6000619, PLACE6000630, PLACE6000953, PLACE6001064, PLACE6001118, PLACE6001153, PLACE6001185, PLACE6001262, PLACE6001281, PLACE6001294, PLACE6001536, PLACE6001557, PLACE6001712, PLACE6001740, PLACE6001823, PLACE6001886, PLACE6001925, PLACE6001933, PLACE6002011, PLACE6002016, PLACE6002056,

PLACE6002102, PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6003004, PLACE6003077, PLACE6003094, PLACE6003109, PLACE6003204, PLACE6003218, PLACE6003372, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004005, PLACE6004101, PLACE6004219, PLACE6004336, PLACE6004368, PLACE6004380, PLACE6004397, PLACE6004454, PLACE6004464, PLACE6004491, PLACE6004578, PLACE6004663, PLACE6004687, PLACE6004738, PLACE6004931, PLACE6004966, PLACE6004993, PLACE6005007,

PLACE6005029, PLACE6005108, PLACE6005231, PLACE6005234, PLACE6005283, PLACE6005294, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006042, PLACE6006077, PLACE6006137, PLACE6006158, PLACE6006186, PLACE6006217, PLACE6006222, PLACE6006266, PLACE6006287, PLACE6006394, PLACE6006418, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007482, PLACE6007687, PLACE6007787, PLACE6007798, PLACE6007807, PLACE6007925, PLACE6008036,

PLACE6008094, PLACE6008126, PLACE6008285, PLACE6008444, PLACE6008453, PLACE6008640, PLACE6008750, PLACE6008768, PLACE6008775, PLACE6008793, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009590, PLACE6009821, PLACE6009835, PLACE6009853, PLACE6010077, PLACE6010463, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011102, PLACE6011156, PLACE6011211, PLACE6011260, PLACE6011627, PLACE6011774, PLACE6011881, PLACE6011907, PLACE6011970, PLACE6011975, PLACE6012028,

PLACE6012108, PLACE6012168, PLACE6012297, PLACE6012308, PLACE6012574, PLACE6012908, PLACE6012936, PLACE6012942, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016254, PLACE6016942, PLACE6016984, PLACE6017002, PLACE6017431, PLACE6017564, PLACE6017578, PLACE6017701, PLACE6017714, PLACE6017788, PLACE6017791, PLACE6018031, PLACE6018107, PLACE6018187,

PLACE6018235, PLACE6018287, PLACE6018441, PLACE6018487, PLACE6018497, PLACE6018769, PLACE6018834, PLACE6018843, PLACE6018938, PLACE6019195, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973,

PLACE7008136, PLACE7008261, PLACE7008431, PLACE7008623, PLACE7008766, PLACE7009563, PROST1000039, PROST1000065, PROST1000083, PROST1000085, PROST1000110, PROST1000123, PROST1000136, PROST1000145, PROST1000152, PROST1000167, PROST1000184, PROST1000199, PROST1000215, PROST1000217, PROST1000220, PROST1000246, PROST1000262, PROST1000272, PROST1000298, PROST1000322, PROST1000334, PROST1000362, PROST1000377, PROST1000383, PROST1000412, PROST1000419, PROST1000451, PROST1000480, PROST1000485, PROST1000493, PROST1000512, PROST1000526, PROST1000527, PROST1000528, PROST1000536, PROST1000559, PROST1000564, PROST1000575, PROST2000053, PROST2000067, PROST2000079, PROST2000105, PROST2000143, PROST2000162,

PROST2000165, PROST2000176, PROST2000203, PROST2000206, PROST2000212, PROST2000241, PROST2000267, PROST2000273, PROST2000274, PROST2000277, PROST2000284, PROST2000325, PROST2000337, PROST2000432, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002, PROST2001116, PROST2001138, PROST2001213, PROST2001283, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001823, PROST2001883, PROST2001899, PROST2001997, PROST2001998, PROST2002078, PROST2002101,

PROST2002162, PROST2002212, PROST2002227, PROST2002338, PROST2002425, PROST2002488, PROST2002489, PROST2002527, PROST2002591, PROST2002602, PROST2002633, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003232, PROST2003302, PROST2003303, PROST2003324, PROST2003340, PROST2003396, PROST2003428, PROST2003472, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251,

PROST2004258, PROST2004332, PROST2004416, PROST2004481, PROST2004526, PROST2004570, PROST2004727, PROST2004739, PROST2004742, PROST2004744, PROST2004817, PROST2005039, PROST2005067, PROST2005076, PROST2005093, PROST2005121, PROST2005131, PROST2005143, PROST2005228, PROST2005272, PROST2005426, PROST2005466, PROST2005630, PROST2005683, PROST2005788, PROST2005880, PROST2005886, PROST2005904, PROST2005919, PROST2005943, PROST2006008, PROST2006020, PROST2006030, PROST2006060, PROST2006196, PROST2006201, PROST2006282, PROST2006343, PROST2006450, PROST2006491, PROST2006510, PROST2006579, PROST2006688, PROST2006766, PROST2006782, PROST2006933, PROST2006988, PROST2007122, PROST2007200, PROST2007237,

PROST2007248, PROST2007289, PROST2007317, PROST2007328, PROST2007382, PROST2007389, PROST2007416, PROST2007528, PROST2007612, PROST2007818, PROST2007871, PROST2007950, PROST2007956, PROST2007974, PROST2007988, PROST2008079, PROST2008088, PROST2008243, PROST2008245, PROST2008268, PROST2008360, PROST2008447, PROST2008468, PROST2008472, PROST2008516, PROST2008724, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318,

PROST2010326, PROST2010382, PROST2010400, PROST2010545, PROST2010579, PROST2010606, PROST2010885, PROST2011012, PROST2011038, PROST2011105, PROST2011297, PROST2011410, PROST2011439, PROST2011482, PROST2011631, PROST2011660, PROST2012005, PROST2012016, PROST2012088, PROST2012140, PROST2012157, PROST2012190, PROST2012249, PROST2012400, PROST2012448, PROST2012504, PROST2012542, PROST2012550, PROST2012740, PROST2012745, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925,

PROST2015124, PROST2015137, PROST2015198, PROST2015243, PROST2015246, PROST2015251, PROST2015332, PROST2015457, PROST2015537, PROST2015545, PROST2015551, PROST2015636, PROST2015710, PROST2015756, PROST2015877, PROST2015900, PROST2015924, PROST2015932, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2016980, PROST2017098, PROST2017128, PROST2017367, PROST2017413, PROST2017441, PROST2017578, PROST2017612, PROST2017617, PROST2017700, PROST2017701, PROST2017729, PROST2017836, PROST2017910, PROST2017972, PROST2018030, PROST2018090,

PROST2018235, PROST2018437, PROST2018487, PROST2018511, PROST2018588, PROST2018607, PROST2018788, PROST2018902, PROST2018922, PROST2018977, PROST2019164, PROST2019253, PROST2019296, PROST2019398, PROST2019487, PROST2019781, PUAEN1000039, PUAEN1000057, PUAEN1000081, PUAEN1000085, PUAEN1000087, PUAEN1000121, PUAEN1000147, PUAEN1000161, PUAEN1000175, PUAEN1000275, PUAEN1000276, PUAEN1000288, PUAEN1000322, PUAEN2000080, PUAEN2000152, PUAEN2000175, PUAEN2000247, PUAEN2000312, PUAEN2000394, PUAEN2000594, PUAEN2000684, PUAEN2000942, PUAEN2001260, PUAEN2001345, PUAEN2001526, PUAEN2001586, PUAEN2001882, PUAEN2002473, PUAEN2002489, PUAEN2002552, PUAEN2002568, PUAEN2002616, PUAEN2002758, PUAEN2003079,

PUAEN2003116, PUAEN2003408, PUAEN2003517, PUAEN2004067, PUAEN2004083, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007044, PUAEN2007350, PUAEN2007785, PUAEN2007898, PUAEN2008123, PUAEN2008228, PUAEN2008314, PUAEN2008515, PUAEN2008815, PUAEN2008980, PUAEN2008985, PUAEN2009174, PUAEN2009348, PUAEN2009534, PUAEN2009655, PUAEN2009795, PUAEN2009852, PUAEN2010346, PUAEN2010824, RECTM1000051, RECTM1000141, RECTM2000220, RECTM2000349, RECTM2000433, RECTM2000510, RECTM2001307, RECTM2001347, RECTM2001519, RECTM2001666, RECTM2001691, RECTM2001749,

RECTM2002172, RECTM2002602, RECTM2002632, SALGL1000005, SALGL1000024, SALGL1000028, SALGL1000047, SALGL1000065, SALGL1000107, SALGL1000171, SKMUS1000014, SKMUS1000022, SKMUS1000030, SKMUS1000064, SKMUS1000067, SKMUS1000084, SKMUS1000102, SKMUS1000104, SKMUS1000110, SKMUS1000118, SKMUS1000124, SKMUS1000129, SKMUS1000138, SKMUS1000176, SKMUS2000020, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000365, SKMUS2000380, SKMUS2000390, SKMUS2000416, SKMUS2000458, SKMUS2000467, SKMUS2000468, SKMUS2000515, SKMUS2000679, SKMUS2000690, SKMUS2000701, SKMUS2000705,

SKMUS2000710, SKMUS2000724, SKMUS2000726, SKMUS2000757, SKMUS2000774, SKMUS2000780, SKMUS2000821, SKMUS2000847, SKMUS2000863, SKMUS2000873, SKMUS2000894, SKMUS2000898, SKMUS2000902, SKMUS2000933, SKMUS2000945, SKMUS2000954, SKMUS2000961, SKMUS2000973, SKMUS2000980, SKMUS2001008, SKMUS2001014, SKMUS2001129, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001501, SKMUS2001543, SKMUS2001580, SKMUS2001585, SKMUS2001608, SKMUS2001631, SKMUS2001662, SKMUS2001663, SKMUS2001668, SKMUS2001671, SKMUS2001683, SKMUS2001693, SKMUS2001695,

SKMUS2001703, SKMUS2001740, SKMUS2001742, SKMUS2001823, SKMUS2001850, SKMUS2002077, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000123, SKNMC1000124, SKNMC1000137, SKNMC1000159, SKNMC1000229,

SKNMC1000251, SKNMC1000264, SKNMC2000224, SKNMC2000256, SKNMC2000305, SKNMC2000356, SKNMC2000374, SKNMC2000583, SKNMC2000612, SKNMC2000622, SKNMC2000877, SKNMC2000966, SKNMC2001113, SKNMC2001324, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000086, SKNSH1000101, SKNSH1000174, SKNSH1000301, SKNSH1000308, SKNSH1000416, SKNSH2000051, SKNSH2000101, SKNSH2000163, SKNSH2000250, SKNSH2000319, SKNSH2000482, SKNSH2000516, SKNSH2000550, SKNSH2000819, SKNSH2001222, SKNSH2001875,

SKNSH2001931, SKNSH2002054, SKNSH2002325, SKNSH2002768, SKNSH2002866, SKNSH2003064, SKNSH2003174, SKNSH2003422, SKNSH2003466, SKNSH2003483, SKNSH2003528, SKNSH2003633, SKNSH2004039, SKNSH2005061, SKNSH2005120, SKNSH2005194, SKNSH2005240, SKNSH2005792, SKNSH2005816, SKNSH2005981, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009357, SKNSH2009435, SKNSH2009991, SKNSH2010015, SMINT1000012, SMINT1000039, SMINT1000042, SMINT1000051, SMINT1000054, SMINT1000057, SMINT1000071, SMINT1000075, SMINT1000078, SMINT1000091, SMINT1000100, SMINT1000103, SMINT1000117, SMINT1000118, SMINT1000137,

SMINT1000192, SMINT2000032, SMINT2000040, SMINT2000060, SMINT2000073, SMINT2000084, SMINT2000145, SMINT2000176, SMINT2000185, SMINT2000227, SMINT2000229, SMINT2000232, SMINT2000267, SMINT2000400, SMINT2000454, SMINT2000468, SMINT2000518, SMINT2000529, SMINT2000530, SMINT2000537, SMINT2000538, SMINT2000541, SMINT2000545, SMINT2000558, SMINT2000562, SMINT2000568, SMINT2000609, SMINT2000629, SMINT2000694, SMINT2000747, SMINT2000811, SMINT2000824, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001818, SMINT2001950,

SMINT2002126, SMINT2002159, SMINT2002202, SMINT2002210, SMINT2002224, SMINT2002281, SMINT2002311, SMINT2002328, SMINT2002414, SMINT2002457, SMINT2002620, SMINT2002689, SMINT2002743, SMINT2002778, SMINT2002880, SMINT2002899, SMINT2002974, SMINT2002976, SMINT2003003, SMINT2003128, SMINT2003150, SMINT2003317, SMINT2003386, SMINT2003423, SMINT2003505, SMINT2003551, SMINT2003569, SMINT2003641, SMINT2003644, SMINT2003866, SMINT2003905, SMINT2004037, SMINT2004047, SMINT2004086, SMINT2004299, SMINT2004339, SMINT2004350, SMINT2004414, SMINT2004473, SMINT2004547, SMINT2004583, SMINT2004589, SMINT2004729, SMINT2004781, SMINT2004872, SMINT2004891, SMINT2004909, SMINT2004972, SMINT2004992, SMINT2005075,

SMINT2005161, SMINT2005174, SMINT2005213, SMINT2005330, SMINT2005387, SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545,

SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2008921, SMINT2009119, SMINT2009121, SMINT2009212, SMINT2009216, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010959, SMINT2010997,

SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012179, SMINT2012195, SMINT2012285, SMINT2012291, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313,

SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000116, SPLEN1000134, SPLEN1000139, SPLEN1000143, SPLEN1000145, SPLEN1000156, SPLEN1000163, SPLEN1000178, SPLEN2000020, SPLEN2000064, SPLEN2000072, SPLEN2000098, SPLEN2000126,

SPLEN2000143, SPLEN2000189, SPLEN2000197, SPLEN2000205, SPLEN2000210, SPLEN2000242, SPLEN2000243, SPLEN2000247, SPLEN2000267, SPLEN2000270, SPLEN2000307, SPLEN2000310, SPLEN2000341, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695, SPLEN2000698, SPLEN2000839, SPLEN2000882, SPLEN2001135, SPLEN2001141, SPLEN2001176, SPLEN2001227, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001689, SPLEN2001710,

SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002335, SPLEN2002343, SPLEN2002354, SPLEN2002385, SPLEN2002419, SPLEN2002451, SPLEN2002462, SPLEN2002463, SPLEN2002477, SPLEN2002493, SPLEN2002662, SPLEN2002695, SPLEN2002707, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004343, SPLEN2004346, SPLEN2004368, SPLEN2004555, SPLEN2004611, SPLEN2004662, SPLEN2004773, SPLEN2004844,

SPLEN2005009, SPLEN2005227, SPLEN2005272, SPLEN2005342, SPLEN2005416, SPLEN2005450, SPLEN2005474, SPLEN2005727, SPLEN2005783, SPLEN2005790, SPLEN2005818, SPLEN2005838, SPLEN2005869, SPLEN2005927, SPLEN2005939, SPLEN2006122, SPLEN2006133, SPLEN2006232, SPLEN2006268, SPLEN2006283, SPLEN2006325, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008496, SPLEN2008591, SPLEN2008786,

SPLEN2009081, SPLEN2009088, SPLEN2009163, SPLEN2009315, SPLEN2009340, SPLEN2009512, SPLEN2009513, SPLEN2009541, SPLEN2009548, SPLEN2009555, SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011419, SPLEN2011422, SPLEN2011672,

SPLEN2011678, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012524, SPLEN2012525, SPLEN2012571, SPLEN2012606, SPLEN2012619, SPLEN2012624, SPLEN2012725, SPLEN2012743, SPLEN2012756, SPLEN2012846, SPLEN2012889, SPLEN2012961, SPLEN2013108, SPLEN2013195, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2014041, SPLEN2014063, SPLEN2014073, SPLEN2014080, SPLEN2014119, SPLEN2014136, SPLEN2014199, SPLEN2014210, SPLEN2014293, SPLEN2014318,

SPLEN2014381, SPLEN2014452, SPLEN2014454, SPLEN2014486, SPLEN2014543, SPLEN2014572, SPLEN2014645, SPLEN2014669, SPLEN2014739, SPLEN2014860, SPLEN2014865, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016268, SPLEN2016304, SPLEN2016356,

SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016773, SPLEN2016781, SPLEN2016840, SPLEN2016863, SPLEN2016922, SPLEN2016932, SPLEN2016973, SPLEN2017043, SPLEN2017104, SPLEN2017121, SPLEN2017147, SPLEN2017187, SPLEN2017189, SPLEN2017212, SPLEN2017318, SPLEN2017426, SPLEN2017452, SPLEN2017538, SPLEN2017592, SPLEN2017613, SPLEN2017643, SPLEN2017687, SPLEN2017740, SPLEN2017781, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732,

SPLEN2018749, SPLEN2018933, SPLEN2019008, SPLEN2019043, SPLEN2019077, SPLEN2019092, SPLEN2019102, SPLEN2019131, SPLEN2019169, SPLEN2019257, SPLEN2019311, SPLEN2019323, SPLEN2019349, SPLEN2019375, SPLEN2019379, SPLEN2019405, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021231, SPLEN2021273, SPLEN2021295,

SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765,

SPLEN2027113, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028994, SPLEN2029051, SPLEN2029176, SPLEN2029295, SPLEN2029380, SPLEN2029522, SPLEN2029683, SPLEN2029727, SPLEN2029904, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098,

SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037891, SPLEN2038055, SPLEN2038180, SPLEN2038345, SPLEN2038407, SPLEN2038834, SPLEN2039311, SPLEN2039379, SPLEN2039672, SPLEN2039697, SPLEN2039845, SPLEN2039936, SPLEN2040222,

SPLEN2040237, SPLEN2040824, SPLEN2041304, SPLEN2041310, SPLEN2041585, SPLEN2041645, SPLEN2041700, SPLEN2041720, SPLEN2041977, SPLEN2042051, SPLEN2042303, SPLEN2042521, SPLEN2042535, SPLEN2042598, SPLEN2042714, STOMA1000013, STOMA1000052, STOMA1000074, STOMA1000091, STOMA1000117, STOMA1000133, STOMA1000175, STOMA1000186, STOMA1000190, STOMA1000191, STOMA2000032, STOMA2000072, STOMA2000088, STOMA2000104, STOMA2000121, STOMA2000148, STOMA2000168, STOMA2000183, STOMA2000188, STOMA2000279, STOMA2000289, STOMA2000383, STOMA2000386, STOMA2000395, STOMA2000396, STOMA2000478, STOMA2000482, STOMA2000539, STOMA2000567, STOMA2000640, STOMA2000678, STOMA2000686, STOMA2000888, STOMA2001025, STOMA2002052,

STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000049, SYNOV1000118,

SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001033, SYNOV2001035, SYNOV2001088, SYNOV2001111, SYNOV2001212, SYNOV2001251, SYNOV2001262, SYNOV2001300, SYNOV2001356, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965,

SYNOV2008765, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542,

SYNOV4007012, SYNOV4007215, SYNOV4007360, SYNOV4007430, SYNOV4007521, SYNOV4007553, SYNOV4007711, SYNOV4008245, SYNOV4008336, SYNOV4009129, SYNOV4009139, SYNOV4009298, SYNOV4009575, T1ESE2000116, T1ESE2000609, T1ESE2000904, T1ESE2002665, TBAES2000004, TBAES2000116, TBAES2000255, TBAES2000315, TBAES2000352, TBAES2000932, TBAES2001171, TBAES2001220, TBAES2001229, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007481, TBAES2007504, TBAES2007548, TBAES2007697, TBAES2007862, TBAES2007964,

TBAES2008133, TCERX2000171, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002273, TESOP2002451, TESOP2002489, TESOP2002539, TESOP2002545, TESOP2002804, TESOP2002950, TESOP2003273, TESOP2003308, TESOP2003553, TESOP2003753,

TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000051, TESTI1000055, TESTI1000064, TESTI1000085,

TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000163, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000172, TESTI2000177, TESTI2000179, TESTI2000184, TESTI2000207, TESTI2000208, TESTI2000221, TESTI2000238, TESTI2000253, TESTI2000272, TESTI2000278, TESTI2000349, TESTI2000356, TESTI2000358,

TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082, TESTI2001134, TESTI2001141, TESTI2001180, TESTI2001208, TESTI2001229, TESTI2001236, TESTI2001237, TESTI2001306, TESTI2001313,

TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001862, TESTI2001869, TESTI2001879, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105,

TESTI2002149, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002365, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002676, TESTI2002690, TESTI2002696, TESTI2002698, TESTI2002700, TESTI2002724, TESTI2002729, TESTI2002752, TESTI2002753, TESTI2002789, TESTI2002802, TESTI2002806, TESTI2002840, TESTI2002877, TESTI2002910, TESTI2002912, TESTI2002928, TESTI2002937, TESTI2002965, TESTI2002993, TESTI2003005, TESTI2003020,

TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003299, TESTI2003311, TESTI2003321, TESTI2003327, TESTI2003330, TESTI2003347, TESTI2003354, TESTI2003356, TESTI2003372, TESTI2003376, TESTI2003413, TESTI2003418, TESTI2003419, TESTI2003432, TESTI2003454, TESTI2003475, TESTI2003476, TESTI2003498, TESTI2003512, TESTI2003539, TESTI2003541, TESTI2003545, TESTI2003551, TESTI2003574, TESTI2003579,

TESTI2003580, TESTI2003589, TESTI2003596, TESTI2003598, TESTI2003615, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004080, TESTI2004085, TESTI2004089, TESTI2004111, TESTI2004119, TESTI2004122, TESTI2004153, TESTI2004163, TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004391, TESTI2004399, TESTI2004423, TESTI2004432, TESTI2004452, TESTI2004459,

TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004734, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994, TESTI2004999, TESTI2005025, TESTI2005040, TESTI2005060, TESTI2005072, TESTI2005085, TESTI2005120, TESTI2005153, TESTI2005155, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415,

TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005759, TESTI2005767, TESTI2005775, TESTI2005784, TESTI2005827, TESTI2005835, TESTI2005860, TESTI2005892, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006051, TESTI2006067, TESTI2006083, TESTI2006109,

TESTI2006111, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006457, TESTI2006460, TESTI2006465, TESTI2006483, TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006774, TESTI2006853, TESTI2006866, TESTI2006872, TESTI2006877, TESTI2006879, TESTI2006894, TESTI2006940,

TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007320, TESTI2007346, TESTI2007358, TESTI2007402, TESTI2007405, TESTI2007407, TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008020, TESTI2008032,

TESTI2008033, TESTI2008046, TESTI2008081, TESTI2008099, TESTI2008139, TESTI2008144, TESTI2008161, TESTI2008189, TESTI2008204, TESTI2008219, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239,

TESTI2009325, TESTI2009388, TESTI2009390, TESTI2009400, TESTI2009402, TESTI2009412, TESTI2009447, TESTI2009462, TESTI2009474, TESTI2009477, TESTI2009497, TESTI2009502, TESTI2009511, TESTI2009544, TESTI2009545, TESTI2009551, TESTI2009585, TESTI2009588, TESTI2009683, TESTI2009727, TESTI2009739, TESTI2009812, TESTI2009835, TESTI2009853, TESTI2009935, TESTI2009987, TESTI2010009, TESTI2010154, TESTI2010180, TESTI2010239, TESTI2010280, TESTI2010354, TESTI2010396, TESTI2010400, TESTI2010409, TESTI2010513, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009,

TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012155, TESTI2012171, TESTI2012207, TESTI2012231, TESTI2012244, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812,

TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257, TESTI2013264, TESTI2013268, TESTI2013295, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339,

TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988, TESTI2015042, TESTI2015092, TESTI2015105, TESTI2015180, TESTI2015246, TESTI2015249, TESTI2015327, TESTI2015331, TESTI2015335, TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421,

TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018014, TESTI2018031, TESTI2018035, TESTI2018060, TESTI2018083, TESTI2018178,

TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018687, TESTI2018697, TESTI2018838, TESTI2018867, TESTI2018941, TESTI2018957, TESTI2018974, TESTI2018975, TESTI2018997, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917,

TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021124, TESTI2021138, TESTI2021297, TESTI2021315, TESTI2021358, TESTI2021425, TESTI2021447, TESTI2021463, TESTI2021531, TESTI2021599, TESTI2021637, TESTI2021654, TESTI2021667, TESTI2021736,

TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968,

TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025454, TESTI2025486, TESTI2025546, TESTI2025582, TESTI2025609, TESTI2025656, TESTI2025757,

TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206,

TESTI2027238, TESTI2027239, TESTI2027243, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252,

TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505,

TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650,

TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041,

TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302,

TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964,

TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137,

TESTI2050681, TESTI2050780, TESTI2050987, TESTI2051148, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288,

TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002072, TESTI4002141, TESTI4002195, TESTI4002202, TESTI4002290, TESTI4002319, TESTI4002491, TESTI4002520, TESTI4002552, TESTI4002647,

TESTI4002703, TESTI4002754, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148,

TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429,

TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009286, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505, TESTI4011532,

TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013602, TESTI4013667, TESTI4013675, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661,

TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961,

TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654,

TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062,

TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432,

TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744,

TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291,

TESTI4044296, TESTI4044300, TESTI4044682, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000002, THYMU1000010, THYMU1000017, THYMU1000025, THYMU1000029, THYMU1000032, THYMU1000033, THYMU1000060, THYMU1000074, THYMU1000083, THYMU1000098, THYMU1000103,

THYMU1000105, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000329, THYMU1000331, THYMU1000336, THYMU1000366, THYMU1000382, THYMU1000393, THYMU1000399, THYMU1000426, THYMU1000440, THYMU1000459, THYMU1000490, THYMU1000491, THYMU1000496, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000120, THYMU2000138, THYMU2000140, THYMU2000155, THYMU2000162, THYMU2000171, THYMU2000176, THYMU2000179, THYMU2000231, THYMU2000236, THYMU2000276,

THYMU2000280, THYMU2000317, THYMU2000368, THYMU2000382, THYMU2000418, THYMU2000440, THYMU2000489, THYMU2000492, THYMU2000519, THYMU2000570, THYMU2000602, THYMU2000660, THYMU2000671, THYMU2000672, THYMU2000684, THYMU2000767, THYMU2000800, THYMU2000879, THYMU2000932, THYMU2000950, THYMU2000971, THYMU2000982, THYMU2000990, THYMU2001007, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001839, THYMU2001868, THYMU2001898,

THYMU2001900, THYMU2001916, THYMU2001926, THYMU2001989, THYMU2002023, THYMU2002037, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002756, THYMU2002789, THYMU2002792, THYMU2002815, THYMU2002841, THYMU2002852, THYMU2002910, THYMU2002950, THYMU2002983, THYMU2003046, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003419, THYMU2003440, THYMU2003470,

THYMU2003479, THYMU2003499, THYMU2003542, THYMU2003632, THYMU2003650, THYMU2003700, THYMU2003760, THYMU2003803, THYMU2003855, THYMU2003907, THYMU2003932, THYMU2004003, THYMU2004008, THYMU2004042, THYMU2004108, THYMU2004111, THYMU2004123, THYMU2004139, THYMU2004152, THYMU2004169, THYMU2004284, THYMU2004320, THYMU2004336, THYMU2004344, THYMU2004356, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283,

THYMU2005303, THYMU2005321, THYMU2005356, THYMU2005369, THYMU2005439, THYMU2005480, THYMU2005482, THYMU2005507, THYMU2005545, THYMU2005546, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006813, THYMU2006913, THYMU2006946, THYMU2006965, THYMU2007025, THYMU2007036, THYMU2007060, THYMU2007065, THYMU2007095, THYMU2007112, THYMU2007146,

THYMU2007179, THYMU2007258, THYMU2007307, THYMU2007308, THYMU2007339, THYMU2007350, THYMU2007384, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007802, THYMU2007824, THYMU2007854, THYMU2007886, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781,

THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010448, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183,

THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012024, THYMU2012073, THYMU2012104, THYMU2012113, THYMU2012273, THYMU2012492, THYMU2012507, THYMU2012565, THYMU2012574, THYMU2012625, THYMU2012631, THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013364,

THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015762, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010,

THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416,

THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249,

THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032035, THYMU2032080, THYMU2032150, THYMU2032155, THYMU2032358, THYMU2032437,

THYMU2032601, THYMU2032655, THYMU2032715, THYMU2032732, THYMU2032825, THYMU2032976, THYMU2033053, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2035735, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226,

THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420,

THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005696, THYMU3006118, THYMU3006132, THYMU3006168, THYMU3006172, THYMU3006371,

THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013386, THYMU3013470, THYMU3013785, THYMU3013897, THYMU3014038, THYMU3014173, THYMU3014372, THYMU3014555, THYMU3014620,

THYMU3014701, THYMU3015042, THYMU3015457, THYMU3015571, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313,

THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032798, THYMU3033402, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200,

THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041573, THYMU3041603, THYMU3041736, THYMU3041918, THYMU3042075, THYMU3042321, THYMU3042372, THYMU3042758, THYMU3042929, THYMU3043200,

THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000066, TKIDN1000164, TKIDN1000171, TKIDN1000192, TKIDN2000127, TKIDN2000240, TKIDN2000246, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2003044, TKIDN2003059, TKIDN2003062,

TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263,

TKIDN2015285, TKIDN2015423, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002046, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528,

TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH100003.8, TRACH1000057, TRACH1000074, TRACH1000100, TRACH1000106, TRACH1000125, TRACH1000140, TRACH1000144, TRACH1000181, TRACH1000193, TRACH1000205, TRACH1000212, TRACH1000240, TRACH2000002, TRACH2000015, TRACH2000024, TRACH2000032, TRACH2000047, TRACH2000081, TRACH2000083, TRACH2000148, TRACH2000185,

TRACH2000196, TRACH2000235, TRACH2000237, TRACH2000243, TRACH2000248, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000420, TRACH2000472, TRACH2000496, TRACH2000497, TRACH2000502, TRACH2000522, TRACH2000540, TRACH2000559, TRACH2000660, TRACH2000665, TRACH2000675, TRACH2000677, TRACH2000703, TRACH2000767, TRACH2000775, TRACH2000807, TRACH2000862, TRACH2000873, TRACH2000894, TRACH2000898, TRACH2000920, TRACH2000923, TRACH2000944, TRACH2000981, TRACH2000986, TRACH2001037, TRACH2001060, TRACH2001085, TRACH2001109, TRACH2001121, TRACH2001147, TRACH2001154, TRACH2001188, TRACH2001192,

TRACH2001249, TRACH2001275, TRACH2001335, TRACH2001339, TRACH2001347, TRACH2001395, TRACH2001400, TRACH2001403, TRACH2001416, TRACH2001428, TRACH2001443, TRACH2001463, TRACH2001474, TRACH2001523, TRACH2001525, TRACH2001532, TRACH2001549, TRACH2001592, TRACH2001596, TRACH2001607, TRACH2001612, TRACH2001621, TRACH2001646, TRACH2001651, TRACH2001898, TRACH2001933, TRACH2001968, TRACH2001996, TRACH2002043, TRACH2002054, TRACH2002100, TRACH2002138, TRACH2002333, TRACH2002784, TRACH2002803, TRACH2002840, TRACH2002853, TRACH2002862, TRACH2002954, TRACH2002960, TRACH2002988, TRACH2003070, TRACH2003323, TRACH2003484, TRACH2003516, TRACH2003605, TRACH2003736, TRACH2003759, TRACH2003941, TRACH2004039,

TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007483, TRACH2007542, TRACH2007577,

TRACH2007674, TRACH2007676, TRACH2007688, TRACH2007733, TRACH2007754, TRACH2007816, TRACH2007894, TRACH2007969, TRACH2007988, TRACH2008081, TRACH2008112, TRACH2008113, TRACH2008127, TRACH2008130, TRACH2008278, TRACH2008300, TRACH2008472, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009310, TRACH2009340, TRACH2009348, TRACH2009358, TRACH2009661, TRACH2009730, TRACH2009743, TRACH2009754, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600,

TRACH2010639, TRACH2010677, TRACH2010698, TRACH2010771, TRACH2010824, TRACH2010883, TRACH2010965, TRACH2010974, TRACH2011057, TRACH2011093, TRACH2011113, TRACH2011290, TRACH2011293, TRACH2011500, TRACH2011574, TRACH2011894, TRACH2011956, TRACH2012000, TRACH2012138, TRACH2012242, TRACH2012298, TRACH2012370, TRACH2012387, TRACH2012497, TRACH2012536, TRACH2012562, TRACH2012742, TRACH2012811, TRACH2012823, TRACH2012918, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014268, TRACH2014284, TRACH2014337,

TRACH2014356, TRACH2014371, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2015180, TRACH2015243, TRACH2015316, TRACH2015381, TRACH2015486, TRACH2015823, TRACH2015824, TRACH2015939, TRACH2015987, TRACH2016080, TRACH2016131, TRACH2016286, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084,

TRACH2018262, TRACH2018273, TRACH2018278, TRACH2018345, TRACH2018380, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479, TRACH2023684, TRACH2023860,

TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000780, TRACH3000892, TRACH3000926, TRACH3001119, TRACH3001443, TRACH3001550, TRACH3001759, TRACH3002064, TRACH3002107,

TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003547, TRACH3003586, TRACH3003683, TRACH3003780, TRACH3003805, TRACH3003832, TRACH3003872, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211,

TRACH3005274, TRACH3005294, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007625, TRACH3007627, TRACH3007689, TRACH3007766, TRACH3007866, TRACH3007995, TRACH3008042, TRACH3008061, TRACH3008201, TRACH3008508, TRACH3008535, TRACH3008629, TRACH3008632, TRACH3008688, TRACH3008697,

TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700,

TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621,

TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148,

TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065,

TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037067, TRACH3037267, TRACH3037288, TRACH3037505, TRACH3037573, TRACH3037696, TRACH3037897, TRACH3038086,

TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN2000046, UMVEN2000069, UMVEN2000121, UMVEN2000133, UMVEN2000152, UMVEN2000453, UTERU1000008,

UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000182, UTERU1000183, UTERU1000187, UTERU1000192, UTERU1000249, UTERU1000337, UTERU1000339, UTERU2000023, UTERU2000047, UTERU2000074, UTERU2000099, UTERU2000154, UTERU2000197, UTERU2000218, UTERU2000238, UTERU2000243, UTERU2000260, UTERU2000300, UTERU2000329, UTERU2000332, UTERU2000338, UTERU2000349, UTERU2000377, UTERU2000393, UTERU2000418, UTERU2000424, UTERU2000465, UTERU2000485, UTERU2000517, UTERU2000524, UTERU2000539, UTERU2000541, UTERU2000542, UTERU2000546, UTERU2000550,

UTERU2000569, UTERU2000607, UTERU2000629, UTERU2000649, UTERU2000663, UTERU2000794, UTERU2000830, UTERU2000844, UTERU2000922, UTERU2001024, UTERU2001110, UTERU2001176, UTERU2001281, UTERU2001389, UTERU2001409, UTERU2001412, UTERU2001504, UTERU2001607, UTERU2001747, UTERU2001876, UTERU2002001, UTERU2002011, UTERU2002176, UTERU2002198, UTERU2002294, UTERU2002332, UTERU2002473, UTERU2002547, UTERU2002662, UTERU2002693, UTERU2002733, UTERU2002736, UTERU2002737, UTERU2002826, UTERU2002841, UTERU2002964, UTERU2002993, UTERU2003035, UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004039,

UTERU2004061, UTERU2004073, UTERU2004163, UTERU2004299, UTERU2004461, UTERU2004520, UTERU2004564, UTERU2004664, UTERU2004688, UTERU2004698, UTERU2004861, UTERU2004929, UTERU2005004, UTERU2005050, UTERU2005069, UTERU2005074, UTERU2005179, UTERU2005292, UTERU2005346, UTERU2005446, UTERU2005449, UTERU2005450, UTERU2005533, UTERU2005601, UTERU2005664, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547, UTERU2006568, UTERU2006593, UTERU2006651, UTERU2006705, UTERU2006899, UTERU2007004, UTERU2007075, UTERU2007081, UTERU2007128, UTERU2007253, UTERU2007267, UTERU2007444,

UTERU2007499, UTERU2007639, UTERU2007924, UTERU2007942, UTERU2008018, UTERU2008019, UTERU2008027, UTERU2008040, UTERU2008077, UTERU2008130, UTERU2008302, UTERU2008347, UTERU2008426, UTERU2008516, UTERU2008561, UTERU2008653, UTERU2008705, UTERU2008707, UTERU2008785, UTERU2008845, UTERU2008901, UTERU2008930, UTERU2008939, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009510, UTERU2009538, UTERU2009540, UTERU2009776, UTERU2009904, UTERU2009951, UTERU2010115, UTERU2010124, UTERU2010164, UTERU2010226, UTERU2010231, UTERU2010304, UTERU2010320, UTERU2010431, UTERU2010525, UTERU2010651,

UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012230, UTERU2012252, UTERU2012333, UTERU2012407, UTERU2012526, UTERU2012581, UTERU2012610, UTERU2012615, UTERU2012688, UTERU2012715, UTERU2012741, UTERU2012786, UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001,

UTERU2014223, UTERU2014398, UTERU2014464, UTERU2014548, UTERU2014601, UTERU2014631, UTERU2014668, UTERU2014678, UTERU2014728, UTERU2014898, UTERU2014998, UTERU2015062, UTERU2015087, UTERU2015108, UTERU2015190, UTERU2015198, UTERU2015202, UTERU2015405, UTERU2015640, UTERU2015830, UTERU2015880, UTERU2016147, UTERU2016157, UTERU2016426, UTERU2016464, UTERU2016669, UTERU2016757, UTERU2016761, UTERU2016822, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514,

UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019005, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023262, UTERU2023550, UTERU2023651, UTERU2023687, UTERU2023712, UTERU2023941,

UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172,

UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844, UTERU3000899, UTERU3000959,

UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460,

UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837,

UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the LIVER, LYMPB, MAMGL, MESAN, MESTC, N1ESE, and NB9N4 libraries were analyzed for expression frequency. The analysis results are described below. Each is a relative value, where the expression level over all tissues is taken as 100. The larger this value, the greater the level of expression.

TABLE 33

| Clone Name | LIVER | LYMPB | MAMGL | MESAN | MESTC | N1ESE | NB9N4 |
|---|---|---|---|---|---|---|---|
| 3NB691000116 | 0 | 0 | 0 | 12.061 | 0 | 0 | 0 |
| 3NB692000281 | 0 | 4.995 | 0 | 0.816 | 0 | 4.999 | 0 |
| 3NB692000912 | 0 | 10.662 | 0 | 0 | 0 | 0 | 0 |
| 3NB692001334 | 0 | 0 | 0 | 0 | 0 | 0 | 9.082 |
| ADIPS2000425 | 0 | 0 | 0 | 0 | 0 | 17.629 | 0 |
| ADRGL2009273 | 0 | 0 | 0 | 5.151 | 0 | 0 | 0 |
| ADRGL2012038 | 0 | 0 | 0 | 0 | 0 | 0 | 69.551 |
| ASTRO1000009 | 0 | 0 | 0 | 0 | 0 | 0 | 88.342 |
| ASTRO2000046 | 0 | 0 | 0 | 4.539 | 0 | 0 | 0 |
| ASTRO2001806 | 0 | 0 | 0 | 5.305 | 0 | 0 | 0 |
| ASTRO2002035 | 0 | 0 | 0 | 40.248 | 0 | 0 | 0 |
| ASTRO2003632 | 0 | 0 | 0 | 15.405 | 0 | 0 | 14.056 |
| ASTRO2004974 | 0 | 0 | 0 | 0 | 0 | 0 | 65.738 |
| ASTRO2005687 | 9.662 | 0 | 0 | 0 | 0 | 0 | 0 |
| ASTRO2009068 | 0 | 0 | 0 | 20.982 | 0 | 0 | 0 |
| ASTRO2010819 | 0 | 0 | 0 | 2.495 | 0 | 0 | 0 |
| ASTRO2012552 | 0 | 0 | 0 | 5.481 | 0 | 0 | 0 |
| ASTRO2013585 | 0 | 0 | 0 | 1.808 | 0 | 0 | 0 |
| ASTRO2015328 | 0 | 0 | 0 | 14.112 | 0 | 0 | 0 |
| BNGH42000360 | 4.090 | 0 | 0 | 0 | 0 | 0 | 0 |
| BNGH42000815 | 0 | 0 | 0 | 32.470 | 0 | 0 | 0 |
| BNGH42005017 | 0 | 0 | 0 | 32.470 | 0 | 0 | 0 |
| BNGH42005968 | 0 | 0 | 0 | 17.034 | 0 | 0 | 0 |
| BNGH42007460 | 0 | 0 | 0 | 7.473 | 0 | 0 | 0 |
| BNGH42007709 | 1.995 | 0 | 0 | 0 | 0 | 0 | 0 |
| BRACE1000115 | 1.623 | 0 | 0 | 0 | 0 | 0 | 0 |
| BRACE2001711 | 33.720 | 0 | 0 | 0 | 0 | 0 | 0 |
| BRACE2002441 | 0 | 0 | 0 | 4.176 | 0 | 0 | 0 |
| BRACE2003172 | 2.817 | 0 | 0 | 0 | 0 | 0 | 0 |
| BRACE2003254 | 0 | 20.545 | 0 | 3.357 | 0 | 0 | 0 |
| BRACE2003846 | 0 | 14.789 | 0 | 0 | 0 | 0 | 0 |
| BRACE2007567 | 0 | 31.488 | 0 | 0 | 0 | 0 | 0 |
| BRACE2007577 | 0 | 0 | 0 | 0 | 84.570 | 0 | 0 |
| BRACE2008358 | 19.643 | 0 | 0 | 0 | 0 | 0 | 0 |
| BRACE2008653 | 0 | 0 | 0 | 3.911 | 0 | 0 | 0 |
| BRACE2008968 | 0 | 13.195 | 0 | 0 | 0 | 0 | 0 |
| BRACE2009037 | 0 | 0 | 0 | 1.083 | 0 | 0 | 0 |
| BRACE2009291 | 11.466 | 0 | 0 | 0 | 0 | 0 | 44.754 |
| BRACE2019147 | 0 | 48.599 | 0 | 0 | 0 | 0 | 0 |
| BRACE2045772 | 0 | 0 | 0 | 0 | 0 | 0 | 97.945 |
| BRACE3004150 | 0 | 0 | 0 | 14.402 | 0 | 0 | 0 |
| BRACE3005938 | 0 | 0 | 0 | 0 | 0 | 25.009 | 0 |
| BRACE3010438 | 0 | 0 | 0 | 6.640 | 0 | 0 | 0 |
| BRAMY2000585 | 0 | 0 | 0 | 0 | 0 | 55.777 | 0 |
| BRAMY2004521 | 0 | 0 | 0 | 17.578 | 0 | 0 | 0 |
| BRAMY2015211 | 0 | 0 | 0 | 48.167 | 0 | 0 | 0 |
| BRAMY2019600 | 0 | 0 | 0 | 29.939 | 0 | 0 | 0 |
| BRAMY2019989 | 0 | 43.763 | 0 | 0 | 0 | 0 | 0 |
| BRAMY2021523 | 0 | 0 | 0 | 78.633 | 0 | 0 | 0 |
| BRAMY2028796 | 0 | 0 | 0 | 0 | 0 | 33.758 | 0 |
| BRAMY2032087 | 0 | 27.482 | 0 | 4.491 | 0 | 0 | 0 |
| BRAMY2040095 | 0 | 0 | 0 | 2.535 | 0 | 0 | 0 |
| BRAMY3004377 | 0 | 0 | 0 | 3.075 | 0 | 0 | 0 |
| BRAMY3010008 | 0 | 0 | 0 | 2.158 | 50.268 | 0 | 19.691 |
| BRAWH1000040 | 0 | 0 | 0 | 2.551 | 0 | 0 | 0 |
| BRAWH1000093 | 0 | 20.933 | 0 | 0 | 0 | 0 | 0 |
| BRAWH2000588 | 0 | 0 | 0 | 4.652 | 0 | 0 | 0 |
| BRAWH2000633 | 0 | 0 | 0 | 4.916 | 0 | 0 | 0 |
| BRAWH2000839 | 0 | 0 | 0 | 6.092 | 0 | 9.327 | 0 |
| BRAWH2000926 | 0 | 0 | 0 | 0 | 0 | 95.782 | 0 |
| BRAWH2001092 | 0 | 0 | 0 | 2.607 | 15.180 | 0 | 5.946 |
| BRAWH2001103 | 56.964 | 0 | 0 | 0 | 0 | 0 | 0 |
| BRAWH2001424 | 0 | 0 | 0 | 18.879 | 0 | 0 | 0 |
| BRAWH2001439 | 0 | 0 | 0 | 3.815 | 0 | 0 | 0 |
| BRAWH2004095 | 0 | 26.826 | 0 | 0 | 0 | 0 | 0 |
| BRAWH2005068 | 0 | 0 | 0 | 0 | 0 | 0 | 13.609 |
| BRAWH2007800 | 0 | 0 | 0 | 0 | 0 | 53.345 | 0 |
| BRAWH2014188 | 0 | 16.246 | 0 | 2.655 | 0 | 0 | 0 |
| BRAWH2014511 | 0 | 0 | 0 | 15.933 | 0 | 0 | 0 |
| BRAWH2016679 | 83.525 | 0 | 0 | 0 | 0 | 0 | 0 |
| BRAWH2017685 | 0 | 0 | 0 | 0 | 0 | 11.525 | 0 |
| BRAWH2018729 | 0 | 67.618 | 0 | 0 | 0 | 0 | 0 |
| BRAWH3016271 | 0 | 0 | 0 | 20 | 0 | 0 | 0 |
| BRAWH3018063 | 0 | 0 | 0 | 19.573 | 0 | 0 | 0 |
| BRCAN2018935 | 79.072 | 0 | 0 | 0 | 0 | 0 | 0 |
| BRHIP2000534 | 0 | 0 | 0 | 3.807 | 0 | 0 | 0 |
| BRHIP2007741 | 0 | 39.391 | 0 | 0 | 0 | 0 | 0 |
| BRHIP2011491 | 0 | 0 | 0 | 0 | 0 | 18.771 | 0 |
| BRHIP2022054 | 0 | 55.882 | 0 | 0 | 0 | 0 | 0 |
| BRHIP2023888 | 0 | 0 | 0 | 2.729 | 0 | 8.356 | 0 |
| BRHIP3007586 | 0 | 0 | 0 | 12.796 | 0 | 0 | 0 |
| BRSSN1000059 | 0 | 0 | 0 | 49.907 | 0 | 0 | 0 |
| BRSSN2001869 | 0 | 0 | 0 | 11.193 | 0 | 0 | 0 |
| BRSTN2011961 | 0 | 0 | 0 | 0 | 0 | 6.577 | 0 |
| BRSTN2012069 | 0 | 0 | 0 | 0.070 | 0.817 | 1.718 | 0 |
| BRSTN2016470 | 1.897 | 0 | 0 | 0.812 | 0 | 0 | 0 |
| BRSTN2016892 | 0 | 67.714 | 0 | 0 | 0 | 0 | 0 |
| BRTHA2007998 | 0 | 0 | 0 | 0 | 74.482 | 0 | 0 |
| BRTHA2010097 | 0 | 0 | 0 | 0 | 0 | 95.344 | 0 |
| BRTHA2017972 | 0 | 0 | 0 | 62.573 | 0 | 0 | 0 |
| BRTHA3003023 | 0 | 0 | 0 | 14.988 | 0 | 45.898 | 0 |
| BRTHA3003074 | 0 | 0 | 0 | 41.752 | 0 | 0 | 0 |
| BRTHA3003736 | 18.647 | 0 | 0 | 0 | 0 | 0 | 0 |
| CD34C3000252 | 0 | 0 | 0 | 3.043 | 0 | 18.640 | 0 |
| CTONG1000087 | 0 | 23.730 | 0 | 0 | 0 | 0 | 0 |
| CTONG1000218 | 0 | 0 | 0 | 1.786 | 41.589 | 0 | 0 |
| CTONG1000241 | 18.389 | 0 | 0 | 7.866 | 0 | 0 | 0 |
| CTONG2000508 | 6.845 | 0 | 0 | 0 | 0 | 0 | 0 |
| CTONG2002073 | 12.697 | 0 | 0 | 0 | 0 | 0 | 0 |
| CTONG2002803 | 0 | 0 | 0 | 5.460 | 0 | 0 | 0 |
| CTONG2003517 | 0 | 0 | 0 | 41.570 | 0 | 0 | 0 |
| CTONG2004487 | 8.199 | 0 | 0 | 0 | 0 | 0 | 0 |
| CTONG2005028 | 0 | 0 | 0 | 25.383 | 0 | 0 | 0 |
| CTONG2005553 | 0 | 16.251 | 0 | 0 | 0 | 0 | 24.229 |
| CTONG2005585 | 0 | 0 | 0 | 0 | 0 | 0 | 94.782 |
| CTONG2006004 | 0 | 0 | 0 | 0 | 0 | 0 | 35.710 |
| CTONG2006235 | 14.200 | 0 | 0 | 0 | 0 | 0 | 0 |
| CTONG2006449 | 0 | 0 | 0 | 32.597 | 0 | 0 | 0 |
| CTONG2006611 | 0 | 0 | 0 | 26.777 | 0 | 0 | 0 |
| CTONG2006932 | 9.539 | 0 | 0 | 0 | 0 | 0 | 0 |
| CTONG2008398 | 0 | 0 | 0 | 2.835 | 0 | 0 | 0 |
| CTONG2010348 | 15.350 | 0 | 0 | 0 | 0 | 0 | 0 |
| CTONG2012158 | 0 | 0 | 0 | 33.828 | 0 | 0 | 0 |
| CTONG2012401 | 0 | 0 | 0 | 15.599 | 0 | 0 | 0 |
| CTONG2012452 | 0 | 0 | 0 | 0 | 0 | 0 | 12.048 |
| CTONG2012879 | 0 | 0 | 0 | 12.610 | 0 | 0 | 0 |
| CTONG2013907 | 0 | 0 | 0 | 7.994 | 0 | 0 | 0 |
| CTONG2013985 | 0 | 0 | 0 | 21.715 | 0 | 0 | 0 |
| CTONG2015091 | 0 | 0 | 0 | 0 | 0 | 18.442 | 0 |
| CTONG2016575 | 0 | 0 | 0 | 1.844 | 0 | 0 | 0 |
| CTONG2017500 | 0 | 0 | 0 | 9.823 | 0 | 0 | 0 |
| CTONG2017998 | 0 | 0 | 0 | 9.289 | 0 | 0 | 0 |
| CTONG2019590 | 0 | 0 | 0 | 39.901 | 0 | 0 | 0 |
| CTONG2019822 | 0 | 73.780 | 0 | 0 | 0 | 0 | 0 |
| CTONG2020484 | 0 | 0 | 0 | 2.180 | 0 | 0 | 0 |
| CTONG2022835 | 0 | 0 | 0 | 6.362 | 0 | 0 | 0 |
| CTONG3008894 | 0 | 0 | 0 | 5.391 | 0 | 0 | 0 |
| DFNES1000150 | 0 | 0 | 0 | 20.705 | 0 | 0 | 0 |
| DFNES2000011 | 0 | 0 | 0 | 0 | 47.230 | 0 | 0 |
| DFNES2000268 | 0 | 25.058 | 0 | 0 | 0 | 0 | 0 |
| DFNES2000292 | 0 | 0 | 0 | 13.916 | 0 | 0 | 0 |
| DFNES2000335 | 0 | 0 | 0 | 17.567 | 0 | 0 | 0 |
| DFNES2007299 | 0 | 74.528 | 0 | 0 | 0 | 0 | 0 |
| FCBBF1000025 | 0 | 0 | 0 | 23.732 | 0 | 0 | 0 |
| FCBBF1000027 | 0 | 0 | 0 | 4.075 | 0 | 0 | 0 |
| FCBBF1000077 | 0 | 0 | 0 | 0 | 0 | 0 | 27.401 |
| FCBBF1000197 | 0 | 0 | 0 | 3.961 | 0 | 0 | 0 |
| FCBBF1000294 | 0 | 0 | 0 | 3.992 | 0 | 0 | 0 |
| FCBBF1000367 | 0 | 0 | 0 | 5.633 | 0 | 0 | 0 |
| FCBBF1000466 | 0 | 14.452 | 0 | 0 | 0 | 0 | 0 |
| FCBBF2000591 | 0 | 0 | 92.989 | 0 | 0 | 0 | 0 |
| FCBBF2001538 | 0 | 0 | 0 | 0 | 0 | 17.303 | 0 |
| FCBBF2005658 | 0 | 0 | 0 | 28.860 | 0 | 0 | 0 |
| FCBBF3003800 | 0 | 0 | 0 | 1.461 | 0 | 0 | 0 |
| FCBBF3004473 | 0 | 0 | 0 | 8.520 | 0 | 0 | 0 |
| FCBBF3005330 | 0 | 0 | 0 | 2.569 | 0 | 0 | 0 |
| FCBBF3005497 | 0 | 17.840 | 0 | 5.830 | 0 | 0 | 0 |
| FCBBF3009541 | 0 | 9.355 | 0 | 1.529 | 0 | 0 | 0 |
| FCBBF3022593 | 0 | 0 | 0 | 25.691 | 0 | 0 | 0 |
| FCBBF3023667 | 1.151 | 0 | 0 | 1.477 | 0 | 0 | 4.493 |
| FCBBF4000446 | 0 | 0 | 0 | 2.785 | 0 | 0 | 0 |
| FEBRA2000105 | 12.434 | 0 | 0 | 0 | 0 | 0 | 0 |
| FEBRA2000581 | 0 | 0 | 0 | 2.950 | 0 | 0 | 0 |
| FEBRA2000733 | 0 | 0 | 0 | 0.625 | 0 | 3.827 | 11.403 |
| FEBRA2002527 | 0 | 64.989 | 0 | 10.620 | 0 | 0 | 0 |
| FEBRA2002933 | 3.208 | 8.398 | 0 | 2.744 | 0 | 0 | 12.520 |
| FEBRA2003822 | 47.676 | 0 | 0 | 0 | 0 | 0 | 0 |
| FEBRA2003833 | 0 | 0 | 0 | 6.412 | 0 | 0 | 0 |
| FEBRA2003926 | 6.051 | 0 | 0 | 2.588 | 0 | 15.853 | 0 |
| FEBRA2004224 | 0 | 0 | 0 | 4.293 | 0 | 0 | 0 |
| FEBRA2005572 | 0 | 40.433 | 0 | 0 | 0 | 0 | 0 |
| FEBRA2005752 | 0 | 0 | 0 | 15.434 | 0 | 0 | 0 |
| FEBRA2006270 | 0 | 0 | 0 | 0 | 0 | 26.548 | 0 |
| FEBRA2006485 | 0 | 0 | 0 | 11.280 | 0 | 0 | 0 |
| FEBRA2007801 | 0 | 0 | 0 | 18.308 | 0 | 0 | 0 |
| FEBRA2008081 | 0 | 0 | 0 | 2.626 | 0 | 0 | 0 |
| FEBRA2008302 | 0 | 0 | 0 | 11.839 | 0 | 0 | 0 |
| FEBRA2008583 | 1.146 | 0 | 0 | 0 | 0 | 0 | 0 |
| FEBRA2009022 | 0 | 0 | 0 | 12.383 | 0 | 0 | 0 |
| FEBRA2019298 | 0 | 0 | 0 | 0 | 0 | 0 | 81.897 |
| FEBRA2022504 | 0 | 0 | 0 | 6.090 | 0 | 0 | 0 |
| FELIV1000153 | 0 | 0 | 0 | 1.223 | 0 | 0 | 5.580 |
| HCASM2000363 | 0 | 0 | 0 | 7.565 | 0 | 0 | 0 |
| HCHON1000015 | 0 | 0 | 0 | 5.780 | 0 | 0 | 0 |
| HCHON1000030 | 0 | 4.116 | 0 | 0.673 | 0 | 4.119 | 0 |
| HCHON1000191 | 0 | 0 | 0 | 0 | 41.718 | 10.969 | 0 |
| HCHON2000028 | 0 | 0 | 0 | 35.299 | 0 | 0 | 0 |
| HCHON2000056 | 0 | 0 | 0 | 3.155 | 0 | 0 | 0 |
| HCHON2000226 | 0 | 0 | 0 | 0 | 0 | 9.158 | 0 |
| HCHON2000244 | 0 | 0 | 0 | 4.878 | 0 | 0 | 0 |
| HCHON2000295 | 0 | 0 | 0 | 0 | 37.831 | 0 | 0 |
| HCHON2000364 | 0 | 0 | 0 | 0 | 38.398 | 10.096 | 0 |
| HCHON2000373 | 0 | 0 | 0 | 15.033 | 0 | 0 | 0 |
| HCHON2000508 | 0 | 0 | 0 | 1.385 | 0 | 0 | 0 |
| HCHON2000676 | 0 | 0 | 0 | 22.111 | 0 | 0 | 0 |
| HCHON2000705 | 0 | 0 | 0 | 36.949 | 0 | 0 | 0 |
| HCHON2000751 | 0 | 0 | 0 | 13.239 | 0 | 0 | 0 |
| HCHON2000819 | 0 | 0 | 0 | 27.167 | 0 | 0 | 0 |
| HCHON2001108 | 0 | 0 | 0 | 0 | 0 | 0 | 72.243 |
| HCHON2001200 | 0 | 9.815 | 0 | 1.604 | 0 | 0 | 0 |
| HCHON2001217 | 0 | 0 | 0 | 7.719 | 0 | 0 | 0 |
| HCHON2001598 | 0 | 0 | 0 | 27.029 | 0 | 0 | 0 |
| HEART2000541 | 11.598 | 0 | 0 | 0 | 0 | 0 | 0 |
| HHDPC1000083 | 0 | 0 | 0 | 0 | 0 | 22.229 | 0 |
| HHDPC2000455 | 0 | 0 | 0 | 14.264 | 47.462 | 12.480 | 0 |
| HHDPC2003439 | 0 | 0 | 0 | 5.659 | 0 | 0 | 0 |
| HHDPC2003983 | 2.414 | 0 | 0 | 0 | 0 | 0 | 0 |
| HLUNG1000064 | 0 | 0 | 0 | 25.250 | 0 | 0 | 0 |
| HLUNG2000004 | 0 | 0 | 0 | 9.379 | 0 | 0 | 0 |
| HLUNG2001507 | 0 | 0 | 0 | 18.568 | 0 | 0 | 0 |
| HLUNG2001518 | 0 | 0 | 0 | 4.055 | 0 | 0 | 0 |
| HLUNG2004154 | 0 | 0 | 0 | 38.561 | 0 | 0 | 0 |
| HLUNG2004388 | 0 | 0 | 0 | 16.557 | 0 | 0 | 0 |
| HLUNG2007210 | 0 | 0 | 0 | 0 | 0 | 0 | 69.546 |
| HSYRA1000062 | 0 | 0 | 0 | 1.588 | 0 | 0 | 0 |
| HSYRA1000119 | 0 | 0 | 0 | 1.705 | 0 | 0 | 0 |
| HSYRA1000152 | 10.339 | 0 | 0 | 0 | 0 | 0 | 0 |
| HSYRA2000159 | 0 | 0 | 0 | 0.447 | 10.413 | 0 | 0 |
| HSYRA2000255 | 0 | 0 | 0 | 1.664 | 0 | 30.570 | 0 |
| HSYRA2000332 | 0 | 0 | 0 | 0 | 0 | 17.713 | 0 |
| HSYRA2000925 | 0 | 0 | 0 | 0.822 | 0 | 5.032 | 0 |
| HSYRA2001103 | 0 | 0 | 0 | 3.145 | 0 | 0 | 0 |
| HSYRA2001138 | 0 | 0 | 0 | 1.891 | 0 | 0 | 0 |
| HSYRA2001225 | 0 | 0 | 0 | 5.482 | 0 | 0 | 0 |
| HSYRA2001476 | 0 | 0 | 0 | 3.580 | 0 | 21.927 | 0 |
| HSYRA2001574 | 0 | 0 | 0 | 0 | 0 | 51.263 | 0 |
| HSYRA2001615 | 0 | 0 | 0 | 7.690 | 0 | 0 | 0 |
| HSYRA2001621 | 0 | 0 | 0 | 1.361 | 0 | 0 | 6.208 |
| HSYRA2007241 | 0 | 0 | 0 | 42.666 | 0 | 0 | 0 |
| HSYRA2007667 | 0 | 0 | 0 | 33.160 | 0 | 0 | 0 |
| IMR321000219 | 0 | 6.369 | 0 | 0 | 0 | 6.374 | 0 |
| IMR321000253 | 0 | 0 | 0 | 0 | 0 | 0 | 34.459 |
| IMR322000072 | 0 | 0 | 0 | 0 | 0 | 0 | 27.147 |
| IMR322000121 | 0 | 0 | 0 | 8.267 | 0 | 0 | 0 |
| IMR322000243 | 0 | 38.938 | 0 | 3.181 | 0 | 0 | 0 |
| IMR322000918 | 0 | 0 | 0 | 10.869 | 0 | 0 | 0 |
| IMR322000935 | 0 | 0 | 0 | 5.552 | 0 | 0 | 0 |
| IMR322000973 | 0 | 18.298 | 0 | 0 | 0 | 0 | 27.281 |
| IMR322001435 | 0 | 0 | 0 | 6.967 | 0 | 0 | 0 |
| IMR322001584 | 0 | 0 | 0 | 4.629 | 0 | 0 | 0 |
| IMR322001600 | 0 | 3.654 | 0 | 0 | 0 | 3.657 | 0 |
| IMR322001710 | 0 | 0 | 0 | 21.575 | 0 | 0 | 0 |
| IMR322012529 | 40.820 | 0 | 0 | 0 | 0 | 0 | 0 |
| KIDNE2000252 | 0 | 19.394 | 0 | 0 | 0 | 0 | 0 |
| KIDNE2000574 | 0 | 0 | 0 | 0 | 0 | 16.999 | 0 |
| KIDNE2001467 | 0 | 0 | 0 | 1.933 | 0 | 0 | 0 |
| KIDNE2002252 | 0 | 49.468 | 0 | 0 | 0 | 0 | 0 |
| KIDNE2002265 | 0 | 0 | 0 | 0 | 0 | 0 | 61.533 |
| KIDNE2002882 | 71.317 | 0 | 0 | 0 | 0 | 0 | 0 |
| KIDNE2003941 | 0 | 0 | 0 | 0 | 43.564 | 0 | 0 |
| KIDNE2004534 | 0 | 0 | 0 | 13.841 | 0 | 0 | 0 |
| KIDNE2006248 | 5.390 | 0 | 0 | 0 | 0 | 0 | 0 |
| KIDNE2006880 | 27.829 | 0 | 0 | 0 | 0 | 0 | 0 |
| KIDNE2008048 | 4.650 | 0 | 0 | 3.978 | 0 | 0 | 0 |
| KIDNE2008116 | 0 | 0 | 0 | 4.302 | 0 | 0 | 0 |
| KIDNE2013845 | 0 | 0 | 0 | 0 | 0 | 25.383 | 0 |
| KIDNE2014290 | 6.077 | 0 | 0 | 0 | 0 | 0 | 0 |
| LIVER1000017 | 54.117 | 0 | 0 | 0 | 0 | 0 | 0 |
| LIVER1000050 | 100 | 0 | 0 | 0 | 0 | 0 | 0 |
| LIVER1000055 | 100 | 0 | 0 | 0 | 0 | 0 | 0 |
| LIVER1000058 | 100 | 0 | 0 | 0 | 0 | 0 | 0 |
| LIVER1000063 | 100 | 0 | 0 | 0 | 0 | 0 | 0 |
| LIVER1000067 | 60.172 | 0 | 0 | 0 | 0 | 0 | 0 |
| LIVER1000073 | 100 | 0 | 0 | 0 | 0 | 0 | 0 |
| LIVER1000079 | 30.377 | 0 | 0 | 0 | 0 | 0 | 0 |
| LIVER1000082 | 100 | 0 | 0 | 0 | 0 | 0 | 0 |
| LIVER1000099 | 100 | 0 | 0 | 0 | 0 | 0 | 0 |
| LIVER1000104 | 21.438 | 0 | 0 | 0 | 0 | 0 | 0 |
| LIVER1000111 | 54.743 | 0 | 0 | 0 | 0 | 0 | 0 |
| LIVER1000126 | 66.455 | 0 | 0 | 0 | 0 | 0 | 0 |
| LIVER1000132 | 4.338 | 0 | 0 | 0 | 0 | 0 | 0 |
| LIVER1000139 | 100 | 0 | 0 | 0 | 0 | 0 | 0 |
| LIVER1000155 | 100 | 0 | 0 | 0 | 0 | 0 | 0 |
| LIVER1000165 | 100 | 0 | 0 | 0 | 0 | 0 | 0 |
| LIVER1000175 | 100 | 0 | 0 | 0 | 0 | 0 | 0 |
| LIVER1000224 | 100 | 0 | 0 | 0 | 0 | 0 | 0 |
| LIVER1000230 | 50.270 | 0 | 0 | 0 | 0 | 0 | 0 |
| LIVER1000278 | 100 | 0 | 0 | 0 | 0 | 0 | 0 |
| LIVER1000303 | 77.605 | 0 | 0 | 0 | 0 | 0 | 0 |
| LIVER1000397 | 100 | 0 | 0 | 0 | 0 | 0 | 0 |
| LIVER1000421 | 71.069 | 0 | 0 | 0 | 0 | 0 | 0 |
| LIVER1000433 | 100 | 0 | 0 | 0 | 0 | 0 | 0 |
| LIVER1000479 | 100 | 0 | 0 | 0 | 0 | 0 | 0 |
| LIVER1000482 | 100 | 0 | 0 | 0 | 0 | 0 | 0 |
| LIVER1000542 | 28.522 | 0 | 0 | 0 | 0 | 0 | 0 |
| LIVER2000003 | 100 | 0 | 0 | 0 | 0 | 0 | 0 |
| LIVER2000033 | 11.991 | 0 | 0 | 10.259 | 0 | 31.416 | 0 |
| LIVER2000037 | 13.711 | 0 | 0 | 0 | 0 | 0 | 0 |
| LIVER2000158 | 59.936 | 0 | 0 | 0 | 0 | 0 | 0 |
| LIVER2000216 | 100 | 0 | 0 | 0 | 0 | 0 | 0 |
| LIVER2000237 | 100 | 0 | 0 | 0 | 0 | 0 | 0 |
| LIVER2000247 | 100 | 0 | 0 | 0 | 0 | 0 | 0 |
| LIVER2000360 | 100 | 0 | 0 | 0 | 0 | 0 | 0 |
| LIVER2000416 | 19.614 | 0 | 0 | 0 | 0 | 0 | 0 |
| LIVER2000446 | 35.657 | 0 | 0 | 0 | 0 | 0 | 0 |
| LIVER2000515 | 100 | 0 | 0 | 0 | 0 | 0 | 0 |
| LIVER2000592 | 100 | 0 | 0 | 0 | 0 | 0 | 0 |
| LIVER2000626 | 100 | 0 | 0 | 0 | 0 | 0 | 0 |
| LIVER2000682 | 100 | 0 | 0 | 0 | 0 | 0 | 0 |
| LIVER2000769 | 100 | 0 | 0 | 0 | 0 | 0 | 0 |
| LIVER2000775 | 100 | 0 | 0 | 0 | 0 | 0 | 0 |
| LIVER2001051 | 100 | 0 | 0 | 0 | 0 | 0 | 0 |
| LIVER2001076 | 100 | 0 | 0 | 0 | 0 | 0 | 0 |
| LIVER2001099 | 100 | 0 | 0 | 0 | 0 | 0 | 0 |
| LIVER2001113 | 92.988 | 0 | 0 | 0 | 0 | 0 | 0 |
| LIVER2001164 | 100 | 0 | 0 | 0 | 0 | 0 | 0 |
| LIVER2001191 | 100 | 0 | 0 | 0 | 0 | 0 | 0 |
| LIVER2001265 | 89.587 | 0 | 0 | 0 | 0 | 0 | 0 |
| LIVER2001329 | 100 | 0 | 0 | 0 | 0 | 0 | 0 |
| LIVER2001389 | 100 | 0 | 0 | 0 | 0 | 0 | 0 |
| LIVER2001461 | 100 | 0 | 0 | 0 | 0 | 0 | 0 |
| LIVER2001539 | 43.167 | 0 | 0 | 0 | 0 | 0 | 0 |
| LIVER2001608 | 22.768 | 0 | 0 | 0 | 0 | 0 | 0 |
| LIVER2001835 | 70.989 | 0 | 0 | 0 | 0 | 0 | 0 |
| LIVER2002638 | 70.623 | 0 | 0 | 0 | 0 | 0 | 0 |
| LIVER2002644 | 100 | 0 | 0 | 0 | 0 | 0 | 0 |
| LIVER2002660 | 100 | 0 | 0 | 0 | 0 | 0 | 0 |
| LIVER2002702 | 100 | 0 | 0 | 0 | 0 | 0 | 0 |
| LIVER2002842 | 16.548 | 0 | 0 | 0 | 0 | 0 | 0 |
| LIVER2003008 | 89.587 | 0 | 0 | 0 | 0 | 0 | 0 |
| LIVER2003065 | 55.769 | 0 | 0 | 0 | 0 | 0 | 0 |
| LIVER2003234 | 87.905 | 0 | 0 | 0 | 0 | 0 | 0 |
| LIVER2003511 | 71.317 | 0 | 0 | 0 | 0 | 0 | 0 |
| LIVER2003524 | 100 | 0 | 0 | 0 | 0 | 0 | 0 |
| LIVER2003568 | 100 | 0 | 0 | 0 | 0 | 0 | 0 |
| LIVER2003581 | 29.021 | 0 | 0 | 0 | 0 | 0 | 0 |
| LIVER2003800 | 9.037 | 0 | 0 | 0 | 0 | 0 | 0 |
| LIVER2003854 | 100 | 0 | 0 | 0 | 0 | 0 | 0 |
| LIVER2004074 | 100 | 0 | 0 | 0 | 0 | 0 | 0 |
| LIVER2004392 | 100 | 0 | 0 | 0 | 0 | 0 | 0 |
| LIVER2004514 | 88.557 | 0 | 0 | 0 | 0 | 0 | 0 |
| LIVER2004565 | 100 | 0 | 0 | 0 | 0 | 0 | 0 |
| LIVER2005028 | 65.496 | 0 | 0 | 0 | 0 | 0 | 0 |
| LIVER2005198 | 100 | 0 | 0 | 0 | 0 | 0 | 0 |
| LIVER2005218 | 42.758 | 0 | 0 | 0 | 0 | 0 | 0 |
| LIVER2005376 | 100 | 0 | 0 | 0 | 0 | 0 | 0 |
| LIVER2005385 | 100 | 0 | 0 | 0 | 0 | 0 | 0 |
| LIVER2005512 | 100 | 0 | 0 | 0 | 0 | 0 | 0 |
| LIVER2005520 | 100 | 0 | 0 | 0 | 0 | 0 | 0 |
| LIVER2005527 | 100 | 0 | 0 | 0 | 0 | 0 | 0 |
| LIVER2005544 | 100 | 0 | 0 | 0 | 0 | 0 | 0 |
| LIVER2005625 | 100 | 0 | 0 | 0 | 0 | 0 | 0 |
| LIVER2005728 | 100 | 0 | 0 | 0 | 0 | 0 | 0 |
| LIVER2005789 | 15.607 | 0 | 0 | 0 | 0 | 0 | 0 |
| LIVER2005973 | 89.470 | 0 | 0 | 0 | 0 | 0 | 0 |
| LIVER2005981 | 24.820 | 0 | 0 | 0 | 0 | 65.025 | 0 |
| LIVER2006006 | 6.164 | 0 | 0 | 5.274 | 0 | 0 | 0 |
| LIVER2006251 | 100 | 0 | 0 | 0 | 0 | 0 | 0 |
| LIVER2006410 | 88.658 | 0 | 0 | 0 | 0 | 0 | 0 |
| LIVER2006469 | 100 | 0 | 0 | 0 | 0 | 0 | 0 |
| LIVER2006644 | 100 | 0 | 0 | 0 | 0 | 0 | 0 |
| LIVER2006764 | 100 | 0 | 0 | 0 | 0 | 0 | 0 |
| LIVER2007415 | 100 | 0 | 0 | 0 | 0 | 0 | 0 |
| LIVER2007548 | 100 | 0 | 0 | 0 | 0 | 0 | 0 |
| LIVER2007568 | 100 | 0 | 0 | 0 | 0 | 0 | 0 |
| LIVER2007721 | 100 | 0 | 0 | 0 | 0 | 0 | 0 |
| LIVER2007783 | 17.786 | 0 | 0 | 0 | 0 | 0 | 0 |
| LIVER2008053 | 100 | 0 | 0 | 0 | 0 | 0 | 0 |
| LIVER2008465 | 57.368 | 0 | 0 | 0 | 0 | 0 | 0 |
| LIVER2008473 | 100 | 0 | 0 | 0 | 0 | 0 | 0 |
| LIVER2008580 | 100 | 0 | 0 | 0 | 0 | 0 | 0 |
| LIVER2008706 | 38.666 | 0 | 0 | 0 | 0 | 0 | 0 |
| LIVER2008751 | 75.266 | 0 | 0 | 0 | 0 | 0 | 0 |
| LIVER2008945 | 100 | 0 | 0 | 0 | 0 | 0 | 0 |
| LIVER2008998 | 100 | 0 | 0 | 0 | 0 | 0 | 0 |
| LIVER2009110 | 100 | 0 | 0 | 0 | 0 | 0 | 0 |
| LIVER2009118 | 100 | 0 | 0 | 0 | 0 | 0 | 0 |
| LIVER2009554 | 100 | 0 | 0 | 0 | 0 | 0 | 0 |
| LYMPB1000141 | 0 | 97.200 | 0 | 0 | 0 | 0 | 0 |
| LYMPB1000158 | 0 | 100 | 0 | 0 | 0 | 0 | 0 |
| LYMPB2000083 | 0 | 100 | 0 | 0 | 0 | 0 | 0 |
| MAMGL1000017 | 0 | 0 | 98.615 | 0 | 0 | 0 | 0 |
| MAMGL1000032 | 0 | 0 | 52.261 | 0 | 0 | 0 | 0 |
| MAMGL1000035 | 0 | 13.709 | 65.316 | 0.747 | 0 | 4.573 | 0 |
| MAMGL1000083 | 0 | 0 | 93.965 | 0 | 0 | 0 | 0 |
| MAMGL1000096 | 0 | 0 | 97.735 | 0 | 0 | 0 | 0 |
| MAMGL1000100 | 0 | 0 | 95.758 | 0 | 0 | 0 | 0 |
| MAMGL1000132 | 0 | 0 | 100 | 0 | 0 | 0 | 0 |
| MAMGL1000151 | 0 | 0 | 100 | 0 | 0 | 0 | 0 |
| MAMGL1000173 | 0 | 0 | 85.972 | 0 | 0 | 0 | 0 |
| MAMGL1000178 | 0 | 0 | 99.213 | 0 | 0 | 0 | 0 |
| MAMGL1000182 | 0 | 0 | 100 | 0 | 0 | 0 | 0 |
| MAMGL1000184 | 0 | 0 | 100 | 0 | 0 | 0 | 0 |
| MESAN1000032 | 0 | 0 | 0 | 76.979 | 0 | 0 | 0 |
| MESAN1000035 | 0 | 0 | 0 | 100 | 0 | 0 | 0 |
| MESAN1000050 | 0 | 0 | 0 | 11.905 | 0 | 0 | 0 |
| MESAN1000079 | 0 | 0 | 0 | 53.297 | 0 | 0 | 0 |
| MESAN1000080 | 0 | 0 | 0 | 10.589 | 0 | 0 | 0 |
| MESAN1000101 | 0 | 0 | 0 | 21.403 | 0 | 0 | 0 |
| MESAN1000121 | 0 | 0 | 0 | 100 | 0 | 0 | 0 |
| MESAN1000126 | 0 | 0 | 0 | 6.622 | 0 | 0 | 0 |
| MESAN1000147 | 0 | 0 | 0 | 38.021 | 0 | 0 | 0 |
| MESAN1000180 | 0 | 0 | 0 | 100 | 0 | 0 | 0 |
| MESAN2000023 | 0 | 0 | 0 | 100 | 0 | 0 | 0 |
| MESAN2000026 | 0 | 0 | 0 | 100 | 0 | 0 | 0 |
| MESAN2000067 | 0 | 0 | 0 | 66.746 | 0 | 0 | 0 |
| MESAN2000092 | 0 | 0 | 0 | 32.812 | 0 | 0 | 0 |
| MESAN2000149 | 0 | 0 | 0 | 9.109 | 0 | 0 | 0 |
| MESAN2000167 | 0 | 0 | 0 | 100 | 0 | 0 | 0 |
| MESAN2000264 | 0 | 0 | 0 | 29.229 | 0 | 0 | 0 |
| MESAN2000267 | 0 | 0 | 0 | 1.518 | 0 | 9.300 | 0 |
| MESAN2000291 | 0 | 0 | 0 | 85.013 | 0 | 0 | 0 |
| MESAN2000337 | 0 | 0 | 0 | 29.402 | 0 | 0 | 0 |
| MESAN2000434 | 0 | 0 | 0 | 85.013 | 0 | 0 | 0 |
| MESAN2000457 | 0 | 0 | 0 | 29.370 | 0 | 0 | 0 |
| MESAN2000462 | 0 | 0 | 0 | 100 | 0 | 0 | 0 |
| MESAN2000501 | 0 | 0 | 0 | 11.125 | 0 | 0 | 0 |
| MESAN2000572 | 0 | 0 | 0 | 7.582 | 0 | 0 | 0 |
| MESAN2000608 | 6.817 | 0 | 0 | 5.832 | 0 | 0 | 0 |
| MESAN2000620 | 0 | 0 | 0 | 67.570 | 0 | 0 | 0 |
| MESAN2000711 | 0 | 0 | 0 | 100 | 0 | 0 | 0 |
| MESAN2000815 | 0 | 0 | 0 | 57.898 | 0 | 0 | 0 |
| MESAN2000894 | 0 | 0 | 0 | 75.663 | 0 | 0 | 0 |
| MESAN2000909 | 0 | 0 | 0 | 61.441 | 0 | 0 | 0 |
| MESAN2001154 | 0 | 0 | 0 | 4.940 | 0 | 0 | 0 |
| MESAN2001450 | 0 | 0 | 0 | 10.694 | 0 | 0 | 0 |
| MESAN2001627 | 0 | 0 | 0 | 48.501 | 0 | 0 | 0 |
| MESAN2001770 | 0 | 0 | 0 | 100 | 0 | 0 | 0 |
| MESAN2001979 | 0 | 0 | 0 | 100 | 0 | 0 | 0 |
| MESAN2001980 | 0 | 0 | 0 | 50.273 | 0 | 0 | 0 |
| MESAN2002086 | 0 | 0 | 0 | 3.087 | 0 | 0 | 0 |
| MESAN2002113 | 0 | 0 | 0 | 100 | 0 | 0 | 0 |
| MESAN2002122 | 0 | 0 | 0 | 32.891 | 0 | 0 | 0 |
| MESAN2002147 | 0 | 0 | 0 | 100 | 0 | 0 | 0 |
| MESAN2002186 | 0 | 0 | 0 | 8.128 | 0 | 0 | 0 |
| MESAN2002424 | 0 | 0 | 0 | 75.663 | 0 | 0 | 0 |
| MESAN2002519 | 0 | 0 | 0 | 17.197 | 0 | 0 | 0 |
| MESAN2002687 | 0 | 0 | 0 | 100 | 0 | 0 | 0 |
| MESAN2002709 | 0 | 0 | 0 | 100 | 0 | 0 | 0 |
| MESAN2002724 | 0 | 0 | 0 | 13.678 | 0 | 0 | 0 |
| MESAN2002790 | 0 | 0 | 0 | 18.138 | 0 | 0 | 0 |
| MESAN2002844 | 0 | 0 | 0 | 26.869 | 0 | 0 | 0 |
| MESAN2002940 | 0 | 0 | 0 | 11.793 | 0 | 0 | 0 |
| MESAN2002978 | 0 | 0 | 0 | 100 | 0 | 0 | 0 |
| MESAN2003035 | 0 | 0 | 0 | 53.150 | 0 | 0 | 0 |
| MESAN2003037 | 0 | 0 | 0 | 100 | 0 | 0 | 0 |
| MESAN2003039 | 0 | 0 | 0 | 60.362 | 0 | 0 | 0 |
| MESAN2003058 | 0 | 0 | 0 | 22.208 | 0 | 0 | 0 |
| MESAN2003101 | 0 | 0 | 0 | 100 | 0 | 0 | 0 |
| MESAN2003190 | 0 | 0 | 0 | 65.620 | 0 | 0 | 0 |
| MESAN2003322 | 0 | 0 | 0 | 100 | 0 | 0 | 0 |
| MESAN2003444 | 0 | 0 | 0 | 13.813 | 0 | 0 | 0 |
| MESAN2003490 | 0 | 0 | 0 | 100 | 0 | 0 | 0 |
| MESAN2003529 | 0 | 0 | 0 | 100 | 0 | 0 | 0 |
| MESAN2003622 | 0 | 0 | 0 | 2.228 | 0 | 0 | 0 |
| MESAN2003646 | 0 | 0 | 0 | 76.979 | 0 | 0 | 0 |
| MESAN2003662 | 0 | 0 | 0 | 100 | 0 | 0 | 0 |
| MESAN2003709 | 0 | 0 | 0 | 30.755 | 0 | 0 | 0 |
| MESAN2003851 | 0 | 0 | 0 | 100 | 0 | 0 | 0 |
| MESAN2004138 | 0 | 0 | 0 | 100 | 0 | 0 | 0 |
| MESAN2004288 | 0 | 0 | 0 | 19.269 | 0 | 0 | 0 |
| MESAN2004575 | 0 | 0 | 0 | 23.931 | 0 | 0 | 0 |
| MESAN2005284 | 0 | 0 | 0 | 17.981 | 0 | 0 | 0 |
| MESAN2005303 | 0 | 0 | 0 | 29.377 | 0 | 0 | 0 |
| MESAN2005371 | 0 | 0 | 0 | 100 | 0 | 0 | 0 |
| MESAN2005633 | 0 | 0 | 0 | 31.828 | 0 | 0 | 0 |
| MESAN2005689 | 0 | 0 | 0 | 23.008 | 0 | 0 | 0 |
| MESAN2005724 | 0 | 0 | 0 | 100 | 0 | 0 | 0 |
| MESAN2005766 | 0 | 0 | 0 | 3.657 | 0 | 0 | 0 |
| MESAN2005808 | 0 | 0 | 0 | 100 | 0 | 0 | 0 |
| MESAN2005811 | 0 | 0 | 0 | 100 | 0 | 0 | 0 |
| MESAN2005957 | 0 | 0 | 0 | 36.616 | 0 | 0 | 0 |
| MESAN2005958 | 0 | 0 | 0 | 9.986 | 0 | 0 | 0 |
| MESAN2006022 | 0 | 0 | 0 | 100 | 0 | 0 | 0 |
| MESAN2006043 | 0 | 0 | 0 | 59.068 | 0 | 0 | 0 |
| MESAN2006328 | 0 | 0 | 0 | 100 | 0 | 0 | 0 |
| MESAN2006401 | 0 | 0 | 0 | 6.708 | 0 | 0 | 0 |
| MESAN2006563 | 0 | 0 | 0 | 5.121 | 0 | 0 | 0 |
| MESAN2006580 | 0 | 0 | 0 | 75.663 | 0 | 0 | 0 |
| MESAN2006599 | 0 | 0 | 0 | 100 | 0 | 0 | 0 |
| MESAN2006743 | 0 | 0 | 0 | 28.586 | 0 | 0 | 0 |
| MESAN2006953 | 0 | 0 | 0 | 25.426 | 0 | 0 | 0 |
| MESAN2006971 | 0 | 0 | 0 | 22.281 | 0 | 0 | 0 |
| MESAN2007032 | 0 | 0 | 0 | 100 | 0 | 0 | 0 |
| MESAN2007440 | 0 | 0 | 0 | 100 | 0 | 0 | 0 |
| MESAN2007597 | 0 | 0 | 0 | 100 | 0 | 0 | 0 |
| MESAN2007755 | 0 | 0 | 0 | 33.922 | 0 | 0 | 0 |
| MESAN2008222 | 0 | 0 | 0 | 100 | 0 | 0 | 0 |
| MESAN2008334 | 0 | 0 | 0 | 30.484 | 0 | 0 | 0 |
| MESAN2008415 | 0 | 0 | 0 | 39.656 | 0 | 0 | 0 |
| MESAN2008460 | 0 | 0 | 0 | 100 | 0 | 0 | 0 |
| MESAN2008536 | 0 | 0 | 0 | 100 | 0 | 0 | 0 |
| MESAN2008679 | 0 | 0 | 0 | 25.983 | 0 | 0 | 0 |
| MESAN2008729 | 0 | 0 | 0 | 83.934 | 0 | 0 | 0 |
| MESAN2008821 | 0 | 0 | 0 | 100 | 0 | 0 | 0 |
| MESAN2008926 | 0 | 72.444 | 0 | 11.838 | 0 | 0 | 0 |
| MESAN2008936 | 0 | 0 | 0 | 14.752 | 0 | 0 | 0 |
| MESAN2009019 | 0 | 0 | 0 | 28.967 | 0 | 0 | 0 |
| MESAN2009081 | 0 | 0 | 0 | 17.454 | 0 | 0 | 0 |
| MESAN2009105 | 0 | 0 | 0 | 100 | 0 | 0 | 0 |
| MESAN2009311 | 0 | 0 | 0 | 100 | 0 | 0 | 0 |
| MESAN2009418 | 0 | 0 | 0 | 100 | 0 | 0 | 0 |
| MESAN2009503 | 0 | 0 | 0 | 61.778 | 0 | 0 | 0 |
| MESAN2009522 | 0 | 0 | 0 | 100 | 0 | 0 | 0 |
| MESAN2009580 | 0 | 0 | 0 | 4.118 | 0 | 0 | 0 |
| MESAN2010031 | 0 | 0 | 0 | 100 | 0 | 0 | 0 |
| MESAN2010114 | 0 | 0 | 0 | 100 | 0 | 0 | 0 |
| MESAN2010312 | 0 | 0 | 0 | 100 | 0 | 0 | 0 |
| MESAN2010321 | 0 | 0 | 0 | 100 | 0 | 0 | 0 |
| MESAN2010664 | 0 | 0 | 0 | 32.181 | 0 | 0 | 0 |
| MESAN2010681 | 0 | 0 | 0 | 100 | 0 | 0 | 0 |
| MESAN2011545 | 0 | 0 | 0 | 100 | 0 | 0 | 0 |
| MESAN2011597 | 0 | 0 | 0 | 100 | 0 | 0 | 0 |
| MESAN2011627 | 0 | 0 | 0 | 13.955 | 0 | 0 | 0 |
| MESAN2011632 | 0 | 0 | 0 | 78.423 | 0 | 0 | 0 |
| MESAN2011977 | 0 | 0 | 0 | 100 | 0 | 0 | 0 |
| MESAN2011984 | 29.577 | 0 | 0 | 6.326 | 0 | 0 | 0 |
| MESAN2012054 | 0 | 0 | 0 | 9.459 | 0 | 0 | 0 |
| MESAN2012113 | 0 | 0 | 0 | 21.877 | 0 | 0 | 0 |
| MESAN2012586 | 0 | 0 | 0 | 100 | 0 | 0 | 0 |
| MESAN2012708 | 0 | 0 | 0 | 100 | 0 | 0 | 0 |
| MESAN2012735 | 0 | 0 | 0 | 36.949 | 0 | 0 | 0 |
| MESAN2013019 | 0 | 0 | 0 | 100 | 0 | 0 | 0 |
| MESAN2013022 | 0 | 0 | 0 | 100 | 0 | 0 | 0 |
| MESAN2013183 | 0 | 0 | 0 | 100 | 0 | 0 | 0 |
| MESAN2013211 | 0 | 0 | 0 | 100 | 0 | 0 | 0 |
| MESAN2013283 | 0 | 0 | 0 | 61.778 | 0 | 0 | 0 |
| MESAN2013284 | 0 | 0 | 0 | 9.108 | 0 | 0 | 0 |
| MESAN2013611 | 0 | 0 | 0 | 100 | 0 | 0 | 0 |
| MESAN2013674 | 0 | 0 | 0 | 100 | 0 | 0 | 0 |
| MESAN2013845 | 0 | 0 | 0 | 41.380 | 0 | 0 | 0 |
| MESAN2013908 | 0 | 0 | 0 | 25.584 | 0 | 0 | 0 |
| MESAN2013936 | 0 | 0 | 0 | 100 | 0 | 0 | 0 |
| MESAN2014192 | 0 | 0 | 0 | 81.641 | 0 | 0 | 0 |
| MESAN2014295 | 0 | 0 | 0 | 75.663 | 0 | 0 | 0 |
| MESAN2014298 | 0 | 0 | 0 | 100 | 0 | 0 | 0 |
| MESAN2014412 | 0 | 0 | 0 | 71.370 | 0 | 0 | 0 |
| MESAN2014624 | 0 | 0 | 0 | 100 | 0 | 0 | 0 |
| MESAN2015277 | 0 | 0 | 0 | 100 | 0 | 0 | 0 |
| MESAN2015365 | 0 | 0 | 0 | 83.934 | 0 | 0 | 0 |
| MESAN2015391 | 0 | 0 | 0 | 74.905 | 0 | 0 | 0 |
| MESAN2015401 | 0 | 0 | 0 | 100 | 0 | 0 | 0 |
| MESAN2015501 | 0 | 0 | 0 | 100 | 0 | 0 | 0 |
| MESAN2015515 | 0 | 0 | 0 | 9.850 | 0 | 0 | 0 |
| MESAN2015708 | 0 | 0 | 0 | 34.496 | 0 | 0 | 0 |
| MESAN2016159 | 0 | 0 | 0 | 85.013 | 0 | 0 | 0 |
| MESAN2016304 | 0 | 0 | 0 | 100 | 0 | 0 | 0 |
| MESAN2016409 | 0 | 0 | 0 | 61.778 | 0 | 0 | 0 |
| MESAN2016552 | 0 | 0 | 0 | 45.115 | 0 | 0 | 0 |
| MESAN2016965 | 0 | 0 | 0 | 100 | 0 | 0 | 0 |
| MESAN2017133 | 0 | 0 | 0 | 32.534 | 0 | 0 | 0 |
| MESAN2017152 | 0 | 0 | 0 | 75.663 | 0 | 0 | 0 |
| MESAN2017373 | 0 | 0 | 0 | 12.988 | 0 | 0 | 0 |
| MESAN2017417 | 0 | 0 | 0 | 100 | 0 | 0 | 0 |
| MESAN2018209 | 0 | 0 | 0 | 100 | 0 | 0 | 0 |
| MESAN2018576 | 0 | 0 | 0 | 39.023 | 0 | 0 | 0 |
| MESAN2018670 | 0 | 0 | 0 | 100 | 0 | 0 | 0 |
| MESAN2018699 | 0 | 0 | 0 | 57.088 | 0 | 0 | 0 |
| MESTC1000042 | 0 | 0 | 0 | 0 | 96.958 | 0 | 0 |
| MESTC1000147 | 0 | 0 | 0 | 0 | 100 | 0 | 0 |
| MESTC2000150 | 0 | 0 | 0 | 0 | 100 | 0 | 0 |
| MESTC2000153 | 0 | 0 | 0 | 0 | 100 | 0 | 0 |
| MESTC2000170 | 0 | 0 | 0 | 0 | 100 | 0 | 0 |
| N1ESE2000698 | 0 | 0 | 0 | 0 | 0 | 96.458 | 0 |
| NB9N41000011 | 0 | 0 | 0 | 0 | 0 | 0 | 72.006 |
| NB9N41000047 | 0 | 0 | 0 | 0 | 0 | 0 | 88.925 |
| NB9N41000121 | 0 | 0 | 0 | 2.785 | 0 | 0 | 50.813 |
| NB9N41000135 | 0 | 0 | 0 | 0 | 0 | 0 | 75.036 |
| NB9N41000142 | 0 | 0 | 0 | 0 | 0 | 0 | 90.579 |
| NB9N41000146 | 0 | 0 | 0 | 0 | 0 | 0 | 100 |
| NB9N41000340 | 0 | 0 | 0 | 0 | 0 | 0 | 49.068 |
| NB9N42000104 | 0 | 18.811 | 0 | 6.147 | 0 | 0 | 56.090 |
| NB9N42000122 | 0 | 0 | 0 | 0 | 0 | 0 | 100 |
| NB9N42000196 | 0 | 0 | 0 | 0 | 0 | 0 | 68.342 |
| NB9N42000281 | 0 | 0 | 0 | 0 | 0 | 0 | 64.692 |
| NB9N42000314 | 0 | 0 | 0 | 0 | 0 | 0 | 100 |
| NB9N42000342 | 0 | 0 | 0 | 10.694 | 0 | 0 | 24.394 |
| NB9N42000430 | 0 | 0 | 0 | 0 | 0 | 0 | 93.650 |
| NB9N42000495 | 0 | 0 | 0 | 0 | 0 | 0 | 100 |
| NB9N42001300 | 0 | 0 | 0 | 0 | 0 | 0 | 91.584 |
| NHNPC2000187 | 0 | 0 | 0 | 0 | 0 | 0 | 15.310 |
| NHNPC2000206 | 0 | 0 | 0 | 5.093 | 0 | 0 | 0 |
| NHNPC2001931 | 0 | 0 | 0 | 5.309 | 0 | 0 | 0 |
| NT2NE1000004 | 0 | 0 | 0 | 30.623 | 0 | 0 | 0 |
| NT2NE1000163 | 0 | 0 | 0 | 1.502 | 0 | 0 | 0 |
| NT2NE2000056 | 0 | 0 | 0 | 2.913 | 0 | 0 | 0 |
| NT2NE2000260 | 0 | 19.189 | 0 | 0.448 | 0 | 0 | 0 |
| NT2NE2000913 | 0 | 0 | 0 | 27.352 | 0 | 0 | 0 |
| NT2NE2001000 | 7.460 | 0 | 0 | 0 | 0 | 0 | 0 |
| NT2NE2001617 | 0 | 0 | 0 | 16.823 | 0 | 0 | 0 |
| NT2NE2001793 | 0 | 0 | 0 | 3.846 | 0 | 0 | 0 |
| NT2NE2003887 | 54.102 | 0 | 0 | 0 | 0 | 0 | 0 |
| NT2NE2004519 | 0 | 0 | 0 | 0 | 0 | 40.694 | 0 |
| NT2NE2005688 | 0 | 0 | 0 | 0 | 64.494 | 0 | 0 |
| NT2NE2010842 | 0 | 21.396 | 0 | 0 | 0 | 0 | 0 |
| NT2NE2014104 | 14.549 | 0 | 0 | 0 | 0 | 0 | 0 |
| NT2RI1000027 | 8.764 | 0 | 0 | 0 | 0 | 0 | 0 |
| NT2RI2000064 | 0 | 0 | 0 | 1.996 | 0 | 0 | 0 |
| NT2RI2000255 | 0 | 0 | 0 | 4.438 | 0 | 0 | 0 |
| NT2RI2000282 | 0 | 0 | 0 | 1.174 | 0 | 0 | 10.709 |
| NT2RI2000284 | 0 | 0 | 0 | 1.690 | 0 | 0 | 0 |
| NT2RI2000344 | 0 | 0 | 0 | 3.002 | 0 | 0 | 0 |
| NT2RI2000578 | 0 | 0 | 0 | 0 | 0 | 7.594 | 22.627 |
| NT2RI2000671 | 0 | 0 | 0 | 0 | 0 | 0 | 35.587 |
| NT2RI2000727 | 0 | 0 | 0 | 3.837 | 0 | 0 | 0 |
| NT2RI2000865 | 0 | 0 | 0 | 0 | 0 | 0 | 94.903 |
| NT2RI2001054 | 0 | 0 | 0 | 2.206 | 0 | 0 | 0 |
| NT2RI2001540 | 0 | 0 | 0 | 0.211 | 0 | 1.293 | 3.852 |
| NT2RI2001657 | 0 | 12.142 | 0 | 7.936 | 0 | 0 | 0 |
| NT2RI2001731 | 0 | 0 | 0 | 14.045 | 0 | 0 | 0 |
| NT2RI2001769 | 0 | 0 | 0 | 0 | 0 | 50.483 | 0 |
| NT2RI2001846 | 0 | 0 | 0 | 18.273 | 0 | 0 | 0 |
| NT2RI2001859 | 0 | 0 | 0 | 7.197 | 0 | 0 | 0 |
| NT2RI2002091 | 0.233 | 0.304 | 0 | 2.487 | 1.158 | 2.741 | 2.269 |
| NT2RI2002152 | 0 | 0 | 0 | 0 | 0 | 0 | 42.547 |
| NT2RI2002316 | 0 | 0 | 0 | 2.973 | 0 | 0 | 0 |
| NT2RI2002530 | 0 | 0 | 0 | 22.382 | 0 | 0 | 0 |
| NT2RI2002654 | 0 | 0 | 0 | 2.178 | 0 | 0 | 0 |
| NT2RI2002822 | 0 | 0 | 0 | 18.605 | 0 | 0 | 0 |
| NT2RI2003051 | 8.874 | 0 | 0 | 0 | 0 | 0 | 0 |
| NT2RI2003304 | 2.275 | 11.910 | 0 | 0.973 | 0 | 5.960 | 0 |
| NT2RI2003407 | 0 | 27.466 | 0 | 4.488 | 0 | 0 | 0 |
| NT2RI2003738 | 3.033 | 3.970 | 0 | 0.649 | 0 | 0 | 0 |
| NT2RI2003751 | 0 | 0 | 0 | 5.137 | 0 | 0 | 0 |
| NT2RI2004188 | 0 | 65.160 | 0 | 0 | 0 | 0 | 0 |
| NT2RI2004284 | 0 | 0 | 0 | 0 | 0 | 0 | 54.386 |
| NT2RI2004884 | 0 | 0 | 0 | 2.865 | 0 | 0 | 0 |
| NT2RI2005115 | 0 | 0 | 0 | 22.659 | 0 | 0 | 0 |
| NT2RI2005116 | 0 | 0 | 0 | 4.740 | 0 | 29.030 | 0 |
| NT2RI2005335 | 0 | 8.784 | 0 | 0 | 0 | 0 | 0 |
| NT2RI2005405 | 0 | 6.370 | 0 | 2.082 | 0 | 0 | 0 |
| NT2RI2005579 | 0 | 0 | 0 | 34.800 | 0 | 0 | 0 |
| NT2RI2005710 | 0 | 0 | 0 | 19.356 | 0 | 0 | 0 |
| NT2RI2005814 | 0 | 29.555 | 0 | 0 | 0 | 0 | 0 |
| NT2RI2006412 | 0 | 0 | 0 | 3.040 | 0 | 0 | 0 |
| NT2RI2006647 | 0 | 0 | 0 | 17.003 | 0 | 0 | 0 |
| NT2RI2007133 | 0 | 0 | 0 | 1.455 | 0 | 0 | 0 |
| NT2RI2007277 | 0 | 22.247 | 0 | 0 | 0 | 0 | 0 |
| NT2RI2007384 | 0 | 0 | 0 | 1.606 | 0 | 0 | 0 |
| NT2RI2007498 | 0 | 0 | 0 | 0 | 35.222 | 0 | 0 |
| NT2RI2007572 | 0 | 0 | 0 | 0 | 0 | 16.968 | 0 |
| NT2RI2007827 | 0 | 0 | 0 | 27.837 | 0 | 0 | 0 |
| NT2RI2007853 | 0 | 0 | 0 | 0 | 0 | 50.846 | 0 |
| NT2RI2008045 | 0 | 0 | 0 | 2.879 | 0 | 0 | 0 |
| NT2RI2008141 | 0 | 0 | 0 | 23.787 | 0 | 0 | 0 |
| NT2RI2008336 | 3.473 | 9.091 | 0 | 1.485 | 0 | 0 | 0 |
| NT2RI2008526 | 0 | 17.398 | 0 | 0 | 0 | 0 | 0 |
| NT2RI2008724 | 0 | 0 | 0 | 0 | 0 | 41.706 | 0 |
| NT2RI2008791 | 0 | 26.476 | 0 | 0 | 0 | 0 | 0 |
| NT2RI2009065 | 4.048 | 0 | 0 | 5.195 | 0 | 10.606 | 0 |
| NT2RI2009233 | 0 | 0 | 0 | 8.364 | 0 | 0 | 0 |
| NT2RI2009583 | 1.939 | 0 | 0 | 0 | 0 | 0 | 0 |
| NT2RI2012350 | 0 | 0 | 0 | 1.484 | 0 | 0 | 0 |
| NT2RI3007543 | 0 | 0 | 0 | 3.146 | 0 | 0 | 0 |
| NT2RP6000072 | 0 | 0 | 0 | 5.992 | 0 | 0 | 0 |
| NT2RP6000077 | 0 | 0 | 0 | 23.835 | 0 | 0 | 0 |
| NT2RP7000238 | 0 | 29.375 | 0 | 0 | 0 | 0 | 0 |
| NT2RP7000624 | 0 | 0 | 0 | 16.178 | 0 | 0 | 0 |
| NT2RP7001166 | 0 | 0 | 0 | 2.773 | 0 | 0 | 0 |
| NT2RP7002379 | 0 | 0 | 0 | 3.733 | 0 | 11.432 | 0 |
| NT2RP7002450 | 0 | 0 | 0 | 0 | 0 | 60.611 | 0 |
| NT2RP7002619 | 0 | 24.725 | 0 | 0 | 0 | 24.743 | 0 |
| NT2RP7002802 | 0 | 0 | 0 | 13.551 | 0 | 0 | 0 |
| NT2RP7002875 | 0 | 0 | 0 | 3.422 | 0 | 0 | 0 |
| NT2RP7003960 | 0 | 0 | 0 | 6.144 | 0 | 0 | 0 |
| NT2RP7004114 | 0 | 0 | 0 | 20.677 | 0 | 0 | 0 |
| NT2RP7004123 | 0 | 43.546 | 0 | 7.116 | 0 | 0 | 0 |
| NT2RP7004173 | 0 | 0 | 0 | 5.664 | 0 | 17.344 | 0 |
| NT2RP7004204 | 0 | 0 | 0 | 6.323 | 0 | 0 | 0 |
| NT2RP7004728 | 0 | 0 | 0 | 5.828 | 0 | 0 | 0 |
| NT2RP7005513 | 0 | 0 | 0 | 0 | 0 | 0 | 57.185 |
| NT2RP7005750 | 0 | 0 | 0 | 13.268 | 0 | 0 | 0 |
| NT2RP7006141 | 32.007 | 0 | 0 | 0 | 0 | 0 | 0 |
| NT2RP7006188 | 0 | 0 | 0 | 9.654 | 0 | 0 | 0 |
| NT2RP7006263 | 0 | 0 | 0 | 14.931 | 0 | 0 | 0 |
| NT2RP7006717 | 0 | 0 | 0 | 7.319 | 0 | 0 | 0 |
| NT2RP7007154 | 4.943 | 0 | 0 | 0 | 0 | 0 | 0 |
| NT2RP7007842 | 0 | 0 | 0 | 7.112 | 0 | 0 | 0 |
| NT2RP7007975 | 0 | 0 | 0 | 10.120 | 0 | 0 | 0 |
| NT2RP7009097 | 0 | 0 | 0 | 6.667 | 0 | 0 | 0 |
| NT2RP7009259 | 0 | 0 | 0 | 2.083 | 0 | 0 | 0 |
| NT2RP7014910 | 0 | 0 | 0 | 0 | 0 | 0 | 40.452 |
| NT2RP7019401 | 0 | 0 | 0 | 38.500 | 0 | 0 | 0 |
| NTONG1000098 | 0 | 0 | 0 | 4.725 | 0 | 0 | 0 |
| NTONG1000264 | 0 | 0 | 0 | 3.614 | 0 | 0 | 0 |
| NTONG2000265 | 0 | 0 | 0 | 6.322 | 0 | 0 | 0 |
| NTONG2000876 | 0 | 0 | 0 | 5.163 | 0 | 0 | 0 |
| NTONG2000977 | 0 | 0 | 0 | 3.645 | 0 | 0 | 0 |
| NTONG2001137 | 9.920 | 0 | 0 | 0 | 0 | 0 | 0 |
| NTONG2001567 | 0 | 0 | 0 | 2.274 | 0 | 6.964 | 0 |
| NTONG2003839 | 0 | 0 | 0 | 9.722 | 0 | 0 | 0 |
| NTONG2004844 | 0 | 0 | 0 | 6.009 | 0 | 0 | 0 |
| NTONG2005520 | 0 | 15.106 | 0 | 2.468 | 0 | 0 | 0 |
| NTONG2006301 | 47.641 | 0 | 0 | 0 | 0 | 0 | 0 |
| NTONG2008483 | 0 | 0 | 0 | 2.963 | 0 | 0 | 0 |
| OCBBF2000013 | 0 | 0 | 0 | 7.215 | 0 | 0 | 0 |
| OCBBF2000522 | 0 | 0 | 0 | 9.376 | 0 | 0 | 0 |
| OCBBF2000712 | 0 | 0 | 0 | 0 | 81.622 | 0 | 0 |
| OCBBF2000846 | 0 | 16.606 | 0 | 0 | 0 | 0 | 0 |
| OCBBF2000982 | 0 | 19.789 | 0 | 0 | 0 | 0 | 0 |
| OCBBF2000986 | 0 | 0 | 0 | 3.573 | 0 | 0 | 0 |
| OCBBF2001687 | 0 | 0 | 0 | 7.133 | 0 | 0 | 0 |
| OCBBF2002626 | 0 | 0 | 0 | 0 | 72.937 | 0 | 0 |
| OCBBF2006849 | 0 | 45.862 | 0 | 0 | 0 | 0 | 0 |
| OCBBF2007224 | 0 | 0 | 0 | 35.012 | 0 | 0 | 0 |
| OCBBF2008041 | 0 | 0 | 0 | 7.751 | 0 | 0 | 0 |
| OCBBF2008822 | 0 | 0 | 0 | 0 | 0 | 0 | 80.650 |
| OCBBF2011021 | 0 | 0 | 0 | 5.360 | 0 | 0 | 0 |
| OCBBF2012553 | 0 | 0 | 0 | 0 | 0 | 26.635 | 0 |
| OCBBF2016841 | 0 | 0 | 0 | 22.652 | 0 | 0 | 0 |
| OCBBF2018873 | 0 | 0 | 0 | 5.138 | 0 | 0 | 0 |
| OCBBF2035226 | 0 | 0 | 0 | 0 | 0 | 0 | 19.876 |
| PEBLM1000174 | 0 | 0 | 0 | 17.733 | 0 | 0 | 0 |
| PEBLM2000222 | 0 | 0 | 0 | 0 | 50.169 | 0 | 0 |
| PEBLM2000267 | 0 | 0 | 0 | 5.214 | 0 | 0 | 0 |
| PEBLM2004452 | 0 | 0 | 0 | 5.005 | 0 | 0 | 0 |
| PEBLM2007774 | 5.222 | 0 | 0 | 0 | 26.013 | 0 | 0 |
| PERIC2005117 | 0 | 0 | 0 | 0 | 0 | 0 | 72.680 |
| PLACE5000068 | 54.438 | 0 | 0 | 0 | 0 | 0 | 0 |
| PLACE5000172 | 22.534 | 0 | 0 | 0 | 0 | 0 | 0 |
| PLACE6000379 | 0 | 12.024 | 0 | 6.550 | 0 | 0 | 0 |
| PLACE6002084 | 0 | 0 | 0 | 2.739 | 0 | 0 | 0 |
| PLACE6006549 | 20.765 | 0 | 0 | 0 | 0 | 0 | 0 |
| PLACE6008793 | 0 | 0 | 0 | 0 | 0 | 0 | 53.643 |
| PLACE6010077 | 0 | 0 | 0 | 3.228 | 0 | 0 | 0 |
| PROST1000144 | 0 | 35.578 | 0 | 0 | 0 | 0 | 0 |
| PROST1000199 | 0 | 0 | 0 | 0 | 0 | 16.593 | 0 |
| PROST1000526 | 0 | 0 | 0 | 9.369 | 0 | 0 | 0 |
| PROST1000564 | 11.652 | 0 | 0 | 4.985 | 0 | 0 | 0 |
| PROST2000138 | 0 | 0 | 0 | 3.857 | 7.486 | 5.905 | 0 |
| PROST2000432 | 0 | 0 | 0 | 51.115 | 0 | 0 | 0 |
| PROST2003517 | 0 | 0 | 0 | 9.762 | 0 | 0 | 0 |
| PROST2004095 | 4.057 | 0 | 0 | 0 | 0 | 0 | 0 |
| PROST2004251 | 0 | 20.330 | 0 | 0 | 0 | 0 | 0 |
| PROST2004270 | 0 | 0 | 0 | 2.180 | 0 | 0 | 0 |
| PROST2004416 | 0 | 0 | 0 | 5.043 | 0 | 0 | 0 |
| PROST2005793 | 0 | 0 | 0 | 14.541 | 0 | 0 | 0 |
| PROST2008360 | 0 | 0 | 0 | 6.674 | 0 | 0 | 0 |
| PROST2008770 | 6.762 | 0 | 0 | 0 | 0 | 0 | 0 |
| PROST2009731 | 0 | 0 | 0 | 0 | 93.666 | 0 | 0 |
| PROST2010782 | 2.108 | 0 | 0 | 0.902 | 0 | 0 | 0 |
| PROST2014916 | 0 | 0 | 0 | 0 | 0 | 82.735 | 0 |
| PROST2017692 | 0 | 0 | 0 | 2.733 | 0 | 16.735 | 0 |
| PUAEN1000164 | 0 | 0 | 0 | 0 | 34.378 | 0 | 0 |
| PUAEN1000322 | 0 | 0 | 0 | 4.740 | 0 | 0 | 0 |
| PUAEN2000175 | 0 | 80.086 | 0 | 0 | 0 | 0 | 0 |
| PUAEN2000374 | 0 | 0 | 0 | 1.187 | 0 | 0 | 10.828 |
| PUAEN2000594 | 0 | 0 | 0 | 39.656 | 0 | 0 | 0 |
| PUAEN2003408 | 0 | 0 | 0 | 11.167 | 0 | 0 | 0 |
| PUAEN2008314 | 0 | 0 | 0 | 39.656 | 0 | 0 | 0 |
| SALGL1000157 | 1.127 | 0 | 0 | 0 | 11.231 | 0 | 0 |
| SKMUS1000022 | 0 | 0 | 0 | 0 | 35.949 | 0 | 0 |
| SKMUS1000186 | 3.479 | 0 | 0 | 0 | 0 | 0 | 0 |
| SKMUS2000484 | 0 | 39.848 | 0 | 0 | 0 | 0 | 0 |
| SKMUS2000679 | 11.615 | 0 | 0 | 0 | 0 | 0 | 0 |
| SKMUS2000931 | 0 | 0 | 0 | 2.018 | 0 | 0 | 0 |
| SKMUS2001454 | 0 | 0 | 0 | 1.252 | 0 | 0 | 11.419 |
| SKNMC1000137 | 9.189 | 0 | 0 | 3.931 | 0 | 0 | 0 |
| SKNMC2000305 | 0 | 0 | 0 | 13.061 | 0 | 0 | 0 |
| SKNMC2000635 | 0 | 0 | 0 | 3.326 | 0 | 8.147 | 0 |
| SKNMC2000877 | 0 | 0 | 0 | 4.394 | 0 | 0 | 0 |
| SKNSH2008940 | 0 | 43.514 | 0 | 0 | 0 | 0 | 0 |
| SMINT1000016 | 21.266 | 0 | 0 | 0 | 0 | 0 | 0 |
| SMINT2000396 | 0 | 0 | 0 | 4.539 | 0 | 0 | 0 |
| SMINT2000400 | 0 | 0 | 0 | 24.805 | 0 | 0 | 0 |
| SMINT2000441 | 0 | 0 | 0 | 1.674 | 0 | 0 | 15.271 |
| SMINT2000694 | 0 | 26.973 | 0 | 0 | 0 | 0 | 0 |
| SMINT2001348 | 0 | 0 | 0 | 34.400 | 0 | 0 | 0 |
| SMINT2001818 | 0 | 21.272 | 0 | 0 | 0 | 0 | 0 |
| SMINT2003169 | 0 | 0 | 0 | 1.744 | 0 | 0 | 0 |
| SMINT2004144 | 55.082 | 0 | 0 | 0 | 0 | 0 | 0 |
| SMINT2008921 | 0 | 25.178 | 0 | 0 | 0 | 0 | 0 |
| SMINT2010639 | 23.481 | 0 | 0 | 0 | 0 | 0 | 0 |
| SPLEN1000106 | 0 | 0 | 0 | 8.631 | 0 | 0 | 0 |
| SPLEN1000116 | 4.290 | 11.231 | 0 | 0 | 0 | 0 | 0 |
| SPLEN2000242 | 83.286 | 0 | 0 | 0 | 0 | 0 | 0 |
| SPLEN2000505 | 0 | 0 | 0 | 4.688 | 0 | 0 | 0 |
| SPLEN2000882 | 0 | 0 | 0 | 13.028 | 0 | 0 | 0 |
| SPLEN2004555 | 0 | 0 | 0 | 3.947 | 0 | 0 | 0 |
| SPLEN2005806 | 0 | 0 | 0 | 0 | 0 | 13.887 | 0 |
| SPLEN2006122 | 0 | 19.112 | 0 | 0 | 0 | 0 | 0 |
| SPLEN2006133 | 0 | 21.527 | 0 | 0 | 0 | 0 | 0 |
| SPLEN2009555 | 0 | 0 | 0 | 31.496 | 0 | 0 | 0 |
| SPLEN2011419 | 0 | 0 | 0 | 8.861 | 0 | 0 | 0 |
| SPLEN2012115 | 0 | 92.880 | 0 | 0 | 0 | 0 | 0 |
| SPLEN2012624 | 43.831 | 0 | 0 | 0 | 0 | 0 | 0 |
| SPLEN2015310 | 4.968 | 0 | 0 | 0 | 0 | 0 | 0 |
| SPLEN2016268 | 0 | 0 | 0 | 1.602 | 0 | 0 | 0 |
| SPLEN2016421 | 0 | 0 | 0 | 7.682 | 0 | 0 | 0 |
| SPLEN2017620 | 0 | 0 | 0 | 19.007 | 0 | 0 | 0 |
| SPLEN2017918 | 0 | 0 | 0 | 6.762 | 0 | 20.705 | 0 |
| SPLEN2027113 | 0 | 0 | 0 | 0 | 0 | 37.161 | 0 |
| SPLEN2028466 | 0 | 0 | 0 | 1.534 | 0 | 0 | 0 |
| SPLEN2036076 | 14.241 | 0 | 0 | 0 | 0 | 0 | 0 |
| STOMA2002166 | 0 | 13.276 | 0 | 0 | 0 | 0 | 0 |
| SYNOV2001033 | 0 | 0 | 0 | 4.446 | 0 | 0 | 0 |
| SYNOV2005541 | 0 | 0 | 0 | 53.086 | 0 | 0 | 0 |
| SYNOV2009172 | 0 | 0 | 0 | 1.930 | 0 | 0 | 0 |
| SYNOV4001153 | 0 | 0 | 0 | 8.600 | 0 | 0 | 0 |
| SYNOV4007671 | 0 | 0 | 0 | 2.718 | 0 | 0 | 0 |
| TBAES2009387 | 0 | 0 | 0 | 0 | 46.168 | 0 | 18.085 |
| TESTI2000253 | 38.706 | 0 | 0 | 0 | 0 | 0 | 0 |
| TESTI2001099 | 0 | 11.120 | 0 | 3.634 | 0 | 0 | 0 |
| TESTI2001269 | 0 | 0 | 0 | 0 | 0 | 24.794 | 0 |
| TESTI2001826 | 0 | 0 | 0 | 41.489 | 0 | 0 | 0 |
| TESTI2002149 | 0 | 37.954 | 0 | 0 | 0 | 0 | 0 |
| TESTI2002365 | 0 | 0 | 0 | 4.049 | 0 | 0 | 0 |
| TESTI2005326 | 0 | 0 | 0 | 85.013 | 0 | 0 | 0 |
| TESTI2005743 | 0 | 0 | 0 | 8.145 | 0 | 0 | 0 |
| TESTI2005759 | 0 | 0 | 0 | 3.468 | 0 | 0 | 0 |
| TESTI2006588 | 0 | 0 | 0 | 2.007 | 0 | 0 | 0 |
| TESTI2007922 | 0 | 89.668 | 0 | 0 | 0 | 0 | 0 |
| TESTI2008240 | 0 | 0 | 0 | 1.855 | 0 | 0 | 0 |
| TESTI2009577 | 0 | 40.108 | 0 | 6.554 | 0 | 0 | 0 |
| TESTI2010513 | 0 | 0 | 0 | 1.457 | 0 | 0 | 0 |
| TESTI2012356 | 0 | 0 | 0 | 0 | 0 | 0 | 98.105 |
| TESTI2012778 | 0 | 8.386 | 0 | 0 | 0 | 0 | 0 |
| TESTI2017954 | 0 | 0 | 0 | 2.456 | 0 | 15.044 | 0 |
| TESTI2018014 | 0 | 0 | 0 | 14.293 | 0 | 0 | 0 |
| TESTI2018997 | 0 | 47.262 | 0 | 0 | 0 | 0 | 0 |
| TESTI2021118 | 92.988 | 0 | 0 | 0 | 0 | 0 | 0 |
| TESTI2033441 | 0 | 0 | 0 | 0 | 0 | 24.920 | 0 |
| TESTI4001467 | 0 | 0 | 0 | 20.529 | 0 | 0 | 0 |
| TESTI4002799 | 0 | 0 | 0 | 1.638 | 0 | 0 | 0 |
| TESTI4013441 | 0 | 0 | 0 | 3.489 | 13.546 | 0 | 0 |
| TESTI4014694 | 0 | 0 | 0 | 0 | 96.252 | 0 | 0 |
| TESTI4037156 | 0 | 6.280 | 0 | 1.026 | 23.900 | 0 | 9.362 |
| TESTI4040598 | 0 | 0 | 0 | 0 | 0 | 97.202 | 0 |
| THYMU1000002 | 0 | 0 | 0 | 6.584 | 0 | 0 | 20.026 |
| THYMU1000010 | 0 | 61.173 | 0 | 0 | 0 | 0 | 0 |
| THYMU1000016 | 0 | 0 | 0 | 0 | 33.377 | 0 | 0 |
| THYMU1000032 | 0 | 17.713 | 0 | 0 | 0 | 0 | 0 |
| THYMU1000359 | 4.647 | 0 | 0 | 0 | 0 | 0 | 0 |
| THYMU1000428 | 4.927 | 0 | 0 | 0 | 0 | 0 | 0 |
| THYMU2000382 | 5.254 | 13.754 | 0 | 0 | 0 | 0 | 0 |
| THYMU2000946 | 0 | 0 | 0 | 16.095 | 0 | 0 | 0 |
| THYMU2001018 | 0 | 0 | 0 | 33.490 | 0 | 0 | 0 |
| THYMU2001422 | 0 | 0 | 0 | 46.185 | 0 | 0 | 0 |
| THYMU2002583 | 0 | 23.522 | 0 | 0 | 0 | 0 | 0 |
| THYMU2002983 | 25.942 | 0 | 0 | 0 | 0 | 0 | 0 |
| THYMU2003891 | 0 | 6.622 | 0 | 0 | 0 | 0 | 0 |
| THYMU2004152 | 8.607 | 0 | 0 | 0 | 0 | 0 | 0 |
| THYMU2004344 | 0 | 28.989 | 0 | 0 | 0 | 0 | 0 |
| THYMU2005546 | 0 | 0 | 0 | 2.028 | 0 | 0 | 0 |
| THYMU2006505 | 0 | 0 | 0 | 10.124 | 0 | 0 | 0 |
| THYMU2007886 | 0 | 0 | 0 | 5.135 | 0 | 0 | 0 |
| THYMU2008185 | 0 | 0 | 0 | 0 | 0 | 0 | 97.595 |
| THYMU2009658 | 0 | 0 | 0 | 1.299 | 0 | 7.955 | 0 |
| THYMU2012625 | 9.005 | 0 | 0 | 0 | 0 | 0 | 0 |
| THYMU2013136 | 0 | 0 | 0 | 0 | 70.643 | 0 | 0 |
| THYMU2013916 | 9.614 | 0 | 0 | 0 | 0 | 0 | 0 |
| THYMU2027125 | 0 | 0 | 0 | 0 | 0 | 0 | 6.056 |
| THYMU2035735 | 0 | 0 | 0 | 4.892 | 0 | 0 | 0 |
| THYMU3038158 | 53.382 | 0 | 0 | 0 | 0 | 0 | 0 |
| TKIDN2000246 | 0 | 0 | 0 | 0 | 0 | 6.908 | 0 |
| TRACH2000472 | 7.402 | 0 | 0 | 0 | 0 | 0 | 0 |
| TRACH2000540 | 12.349 | 0 | 0 | 0 | 0 | 0 | 0 |
| TRACH2000559 | 0 | 0 | 0 | 0 | 0 | 88.443 | 0 |
| TRACH2001021 | 3.630 | 0 | 0 | 0 | 0 | 0 | 0 |
| TRACH2001289 | 0 | 0 | 0 | 0 | 29.356 | 0 | 0 |
| TRACH2001478 | 0 | 0 | 0 | 2.153 | 50.138 | 0 | 0 |
| TRACH2001607 | 8.706 | 0 | 0 | 0 | 0 | 0 | 0 |
| TRACH2001810 | 0 | 0 | 0 | 0 | 0 | 0 | 26.274 |
| TRACH2001898 | 0 | 0 | 0 | 0 | 88.050 | 0 | 0 |
| TRACH2002092 | 0 | 0 | 0 | 20.818 | 0 | 0 | 0 |
| TRACH2004499 | 0 | 0 | 0 | 0 | 0 | 66.734 | 0 |
| TRACH2005800 | 0 | 0 | 0 | 0 | 0 | 54.636 | 0 |
| TRACH2012298 | 0 | 0 | 0 | 0 | 0 | 0 | 48.010 |
| TRACH3007479 | 0 | 0 | 0 | 11.964 | 0 | 0 | 0 |
| TRACH3027229 | 93.931 | 0 | 0 | 0 | 0 | 0 | 0 |
| UMVEN1000186 | 0 | 0 | 0 | 5.878 | 0 | 0 | 0 |
| UMVEN2000133 | 0 | 13.301 | 0 | 0 | 0 | 0 | 0 |
| UTERU2000095 | 0 | 0 | 0 | 1.535 | 0 | 0 | 0 |
| UTERU2000263 | 0 | 0 | 0 | 0 | 44.254 | 0 | 0 |
| UTERU2001607 | 0 | 33.475 | 0 | 5.470 | 0 | 0 | 0 |
| UTERU2002294 | 15.736 | 0 | 0 | 0 | 0 | 0 | 0 |
| UTERU2002662 | 0 | 0 | 0 | 6.598 | 0 | 0 | 0 |
| UTERU2005593 | 0 | 0 | 0 | 4.162 | 0 | 0 | 0 |
| UTERU2005621 | 19.540 | 0 | 0 | 0 | 0 | 0 | 0 |
| UTERU2007253 | 0 | 0 | 0 | 38.210 | 0 | 0 | 0 |
| UTERU2008085 | 0 | 0 | 0 | 0.335 | 0 | 2.051 | 3.055 |
| UTERU2008302 | 0 | 0 | 0 | 0 | 0 | 30.340 | 0 |
| UTERU2010417 | 0 | 0 | 0 | 1.169 | 0 | 0 | 0 |
| UTERU2011220 | 0 | 88.827 | 0 | 0 | 0 | 0 | 0 |
| UTERU2015108 | 0 | 0 | 0 | 23.399 | 0 | 0 | 0 |
| UTERU2018566 | 0 | 0 | 0 | 66.092 | 0 | 0 | 0 |
| UTERU2019005 | 0 | 0 | 0 | 23.949 | 0 | 0 | 0 |

Genes whose expression levels were negligibly low in each of the above-described libraries are shown with their clone names below.

3NB691000018, 3NB691000085, 3NB691000113, 3NB691000129, 3NB691000173, 3NB691000191, 3NB692000012, 3NB692000029, 3NB692000102, 3NB692000154, 3NB692000276, 3NB692000305, 3NB692000330, 3NB692000374, 3NB692000429, 3NB692000484, 3NB692000529, 3NB692000545, 3NB692000973, 3NB692001002, 3NB692001022, 3NB692001034, 3NB692001040, 3NB692001123, 3NB692001267, 3NB692001288, 3NB692001339, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001520, 3NB692001528, 3NB692001538, 3NB692001548, 3NB692001557, 3NB692001637, 3NB692001719, 3NB692001853, 3NB692001861, 3NB692002051, 3NB692002365,

3NB692002685, 3NB692002806, 3NB692003538, 3NB692004045, 3NB692004724, 3NB692005439, 3NB692006952, 3NB692008178, 3NB692008729, ACTVT2000380, ADIPS1000064, ADIPS2000069, ADIPS2000088, ADIPS2000245, ADRGL1000002, ADRGL1000018, ADRGL1000033, ADRGL1000038, ADRGL1000065, ADRGL1000067, ADRGL1000111, ADRGL1000144, ADRGL1000147, ADRGL1000160, ADRGL1000165, ADRGL1000182, ADRGL2000006, ADRGL2000042, ADRGL2000056, ADRGL2000064, ADRGL2000074, ADRGL2000085, ADRGL2000097, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301,

ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002477, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817,

ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012179, AHMSC1000138, ASTRO1000018, ASTRO1000029, ASTRO1000096, ASTRO1000165, ASTRO2000011, ASTRO2000014, ASTRO2000095, ASTRO2000141, ASTRO2000191, ASTRO2000278, ASTRO2000372, ASTRO2000381, ASTRO2000403, ASTRO2000417, ASTRO2000480, ASTRO2000482, ASTRO2000533,

ASTRO2000653, ASTRO2000662, ASTRO2000725, ASTRO2000801, ASTRO2000914, ASTRO2001001, ASTRO2001008, ASTRO2001029, ASTRO2001076, ASTRO2001088, ASTRO2001107, ASTRO2001122, ASTRO2001227, ASTRO2001249, ASTRO2001480, ASTRO2001823, ASTRO2002024, ASTRO2002064, ASTRO2002202, ASTRO2002459, ASTRO2002571, ASTRO2002632, ASTRO2002659, ASTRO2002693, ASTRO2002720, ASTRO2002733, ASTRO2002743, ASTRO2002842, ASTRO2003024, ASTRO2003212, ASTRO2003316, ASTRO2003461, ASTRO2003574, ASTRO2003581, ASTRO2003740, ASTRO2003840, ASTRO2003925, ASTRO2003960, ASTRO2004032, ASTRO2004051, ASTRO2004177, ASTRO2004217, ASTRO2004297, ASTRO2004584, ASTRO2004628, ASTRO2004751, ASTRO2004877, ASTRO2005008, ASTRO2005081, ASTRO2005096,

ASTRO2005242, ASTRO2005273, ASTRO2005343, ASTRO2005360, ASTRO2005413, ASTRO2005423, ASTRO2005553, ASTRO2005557, ASTRO2005575, ASTRO2005593, ASTRO2005726, ASTRO2005863, ASTRO2005896, ASTRO2005902, ASTRO2006356, ASTRO2006475, ASTRO2006732, ASTRO2006747, ASTRO2006920, ASTRO2006952, ASTRO2007047, ASTRO2007221, ASTRO2007377, ASTRO2007515, ASTRO2007578, ASTRO2007643, ASTRO2007666, ASTRO2007948, ASTRO2007956, ASTRO2007962, ASTRO2008040, ASTRO2008216, ASTRO2008239, ASTRO2008409, ASTRO2008425, ASTRO2008508, ASTRO2008777, ASTRO2008895, ASTRO2008960, ASTRO2008972, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799,

ASTRO2010962, ASTRO2010996, ASTRO2011149, ASTRO2011422, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014863, ASTRO2014923, ASTRO2014961, ASTRO2015162, ASTRO2015185, ASTRO2015529, ASTRO2015987, ASTRO2016044, ASTRO2016114, ASTRO2016148, ASTRO2016491, ASTRO2016681, ASTRO2016819, ASTRO2016847, ASTRO2016892, ASTRO2017348, ASTRO2017627, ASTRO2018169, ASTRO2018373, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000123,

BGGI11000193, BGGI11000285, BGGI12000022, BGGI12000054, BGGI12000067, BGGI12000161, BGGI12000533, BGGI12000544, BGGI12000616, BGGI12000684, BGGI12000693, BGGI12000839, BGGI12001002, BGGI12001075, BGGI12001097, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474,

BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006830, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000137, BNGH41000153, BNGH41000169,

BNGH41000177, BNGH41000190, BNGH41000198, BNGH41000216, BNGH41000235, BNGH42000130, BNGH42000132, BNGH42000405, BNGH42000532, BNGH42000675, BNGH42000739, BNGH42000791, BNGH42000994, BNGH42001247, BNGH42001406, BNGH42001576, BNGH42001724, BNGH42002168, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005235, BNGH42005504, BNGH42006135, BNGH42006226, BNGH42006234, BNGH42007037, BNGH42007366, BNGH42007594, BNGH42007788, BNGH42007798, BNGH42008199, BNGH42008510, BNGH42008603, BNGH42008649, BNGH42008743, BNGH42008832, BNGH42008850, BNGH42008943, BNGH42009021, BNGH42009025, BRACE1000020,

BRACE1000042, BRACE1000047, BRACE1000051, BRACE1000065, BRACE1000070, BRACE1000073, BRACE1000084, BRACE1000087, BRACE1000092, BRACE1000093, BRACE1000101, BRACE1000114, BRACE1000124, BRACE1000131, BRACE1000132, BRACE1000159, BRACE1000166, BRACE1000169, BRACE1000186, BRACE1000187, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000307, BRACE2000331, BRACE2000332, BRACE2000347, BRACE2000351, BRACE2000368, BRACE2000392, BRACE2000441, BRACE2000487,

BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000753, BRACE2000815, BRACE2000864, BRACE2000885, BRACE2000893, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001117, BRACE2001143, BRACE2001151, BRACE2001154, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353,

BRACE2001374, BRACE2001375, BRACE2001423, BRACE2001424, BRACE2001445, BRACE2001453, BRACE2001455, BRACE2001477, BRACE2001492, BRACE2001496, BRACE2001497, BRACE2001508, BRACE2001534, BRACE2001543, BRACE2001564, BRACE2001588, BRACE2001607, BRACE2001626, BRACE2001673, BRACE2001677, BRACE2001692, BRACE2001705, BRACE2001709, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001892, BRACE2001898, BRACE2001923, BRACE2001942, BRACE2001944, BRACE2001954, BRACE2001971, BRACE2001972, BRACE2001973, BRACE2002026, BRACE2002034, BRACE2002050, BRACE2002081, BRACE2002091, BRACE2002139,

BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002392, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002661, BRACE2002676, BRACE2002677, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002807, BRACE2002812, BRACE2002834,

BRACE2002849, BRACE2002857, BRACE2002860, BRACE2002861, BRACE2002884, BRACE2002896, BRACE2002948, BRACE2003037, BRACE2003078, BRACE2003097, BRACE2003110, BRACE2003168, BRACE2003199, BRACE2003210, BRACE2003217, BRACE2003269, BRACE2003285, BRACE2003304, BRACE2003319, BRACE2003398, BRACE2003420, BRACE2003428, BRACE2003431, BRACE2003512, BRACE2003516, BRACE2003527, BRACE2003539, BRACE2003540, BRACE2003594, BRACE2003598, BRACE2003609, BRACE2003614, BRACE2003628, BRACE2003639, BRACE2003700, BRACE2003713, BRACE2003766, BRACE2003785, BRACE2003788, BRACE2003800, BRACE2003802, BRACE2003825, BRACE2003827, BRACE2003845, BRACE2003847, BRACE2003848, BRACE2003873, BRACE2003885, BRACE2003887, BRACE2003892,

BRACE2003904, BRACE2003905, BRACE2003928, BRACE2003944, BRACE2003954, BRACE2003982, BRACE2003987, BRACE2005034, BRACE2005047, BRACE2005063, BRACE2005083, BRACE2005087, BRACE2005090, BRACE2005099, BRACE2005118, BRACE2005124, BRACE2005138, BRACE2005151, BRACE2005160, BRACE2005169, BRACE2005189, BRACE2005193, BRACE2005196, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005433, BRACE2005434, BRACE2005448, BRACE2005450, BRACE2005453, BRACE2005457, BRACE2005460, BRACE2005489, BRACE2005518, BRACE2005556, BRACE2005562, BRACE2005624,

BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006055, BRACE2006072, BRACE2006075, BRACE2006084, BRACE2006089, BRACE2006102, BRACE2006105, BRACE2006122, BRACE2006137, BRACE2006143, BRACE2006162, BRACE2006167, BRACE2006174, BRACE2006240, BRACE2006245, BRACE2006249, BRACE2006258, BRACE2006264, BRACE2006274, BRACE2006281, BRACE2006298, BRACE2006319, BRACE2006344, BRACE2006354, BRACE2006363,

BRACE2006378, BRACE2006380, BRACE2006393, BRACE2006397, BRACE2006413, BRACE2006417, BRACE2006453, BRACE2006459, BRACE2006488, BRACE2006514, BRACE2006534, BRACE2006537, BRACE2006540, BRACE2006547, BRACE2006564, BRACE2006587, BRACE2006598, BRACE2006636, BRACE2006685, BRACE2006703, BRACE2006743, BRACE2006753, BRACE2006783, BRACE2006821, BRACE2006833, BRACE2006859, BRACE2006871, BRACE2006900, BRACE2006909, BRACE2006911, BRACE2006924, BRACE2006935, BRACE2006937, BRACE2006944, BRACE2006951, BRACE2006982, BRACE2006986, BRACE2007013, BRACE2007064, BRACE2007068, BRACE2007087, BRACE2007089, BRACE2007109, BRACE2007138, BRACE2007174, BRACE2007191, BRACE2007197, BRACE2007201, BRACE2007203, BRACE2007232,

BRACE2007254, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007518, BRACE2007522, BRACE2007527, BRACE2007538, BRACE2007563, BRACE2007621, BRACE2007640, BRACE2007641, BRACE2007646, BRACE2007663, BRACE2007671, BRACE2007682, BRACE2007685, BRACE2007708, BRACE2007727, BRACE2007749, BRACE2007760, BRACE2007761, BRACE2007764, BRACE2007767, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902, BRACE2007920, BRACE2007937, BRACE2007944,

BRACE2007952, BRACE2008008, BRACE2008015, BRACE2008021, BRACE2008097, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008361, BRACE2008380, BRACE2008385, BRACE2008399, BRACE2008401, BRACE2008402, BRACE2008410, BRACE2008420, BRACE2008443, BRACE2008473, BRACE2008480, BRACE2008481, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008706, BRACE2008708, BRACE2008710, BRACE2008754, BRACE2008797, BRACE2008847, BRACE2008857, BRACE2008861, BRACE2008867, BRACE2008881, BRACE2008883,

BRACE2008917, BRACE2008919, BRACE2008941, BRACE2008948, BRACE2008960, BRACE2008999, BRACE2009014, BRACE2009016, BRACE2009031, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009517, BRACE2009533, BRACE2009558, BRACE2009566, BRACE2009591, BRACE2009620, BRACE2009654, BRACE2009721, BRACE2009732, BRACE2009754, BRACE2009828, BRACE2009886, BRACE2009907, BRACE2009957,

BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317,

BRACE2012510, BRACE2012528, BRACE2012625, BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194,

BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017397, BRACE2017410, BRACE2017438, BRACE2017574, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019244, BRACE2019258, BRACE2019327, BRACE2019348, BRACE2019371, BRACE2019467, BRACE2019510, BRACE2019519,

BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021014, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549,

BRACE2022633, BRACE2022638, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258,

BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584,

BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600,

BRACE2039607, BRACE2039624, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045947, BRACE2045954, BRACE2046251,

BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046,

BRACE3004058, BRACE3004113, BRACE3004205, BRACE3004358, BRACE3004371, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036,

BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010428, BRACE3010435, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418,

BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594,

BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241,

BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358,

BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988,

BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009482, BRALZ2009486, BRALZ2010186, BRALZ2010842, BRALZ2011337, BRALZ2011796, BRALZ2011880, BRALZ2012050,

BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000354, BRAMY2000388, BRAMY2000411, BRAMY2000508, BRAMY2000562, BRAMY2000653, BRAMY2000814, BRAMY2000893, BRAMY2000946, BRAMY2000981, BRAMY2000987, BRAMY2001068, BRAMY2001114, BRAMY2001203,

BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004363, BRAMY2004387,

BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653,

BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480,

BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012536, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813,

BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016325, BRAMY2016327, BRAMY2016386, BRAMY2016706, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055,

BRAMY2019111, BRAMY2019277, BRAMY2019300, BRAMY2019468, BRAMY2019509, BRAMY2019554, BRAMY2019643, BRAMY2019710, BRAMY2019963, BRAMY2019985, BRAMY2020050, BRAMY2020058, BRAMY2020106, BRAMY2020270, BRAMY2020354, BRAMY2020355, BRAMY2020412, BRAMY2020427, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493,

BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2023939, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218,

BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027739, BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028740, BRAMY2028783,

BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993,

BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214,

BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420,

BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3002991, BRAMY3003026, BRAMY3003109, BRAMY3003205, BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078,

BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010492, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000007, BRAWH1000037,

BRAWH1000045, BRAWH1000083, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417, BRAWH2000443, BRAWH2000460, BRAWH2000471, BRAWH2000476, BRAWH2000482, BRAWH2000488, BRAWH2000494, BRAWH2000500, BRAWH2000503, BRAWH2000522, BRAWH2000554, BRAWH2000595, BRAWH2000636, BRAWH2000643, BRAWH2000651, BRAWH2000682, BRAWH2000686,

BRAWH2000697, BRAWH2000752, BRAWH2000859, BRAWH2000866, BRAWH2000892, BRAWH2000901, BRAWH2000924, BRAWH2000944, BRAWH2000984, BRAWH2001019, BRAWH2001037, BRAWH2001052, BRAWH2001129, BRAWH2001137, BRAWH2001141, BRAWH2001159, BRAWH2001166, BRAWH2001171, BRAWH2001186, BRAWH2001203, BRAWH2001238, BRAWH2001239, BRAWH2001255, BRAWH2001321, BRAWH2001355, BRAWH2001378, BRAWH2001395, BRAWH2001406, BRAWH2001412, BRAWH2001418, BRAWH2001436, BRAWH2001438, BRAWH2001459, BRAWH2001461, BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686, BRAWH2001701, BRAWH2001842, BRAWH2001862, BRAWH2001873,

BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003025, BRAWH2003135, BRAWH2003240, BRAWH2003307, BRAWH2003355, BRAWH2003366, BRAWH2003662, BRAWH2003689, BRAWH2003693, BRAWH2003818, BRAWH2003832, BRAWH2003948, BRAWH2003964, BRAWH2004044, BRAWH2004068, BRAWH2004078, BRAWH2004136, BRAWH2004217, BRAWH2004580, BRAWH2004731, BRAWH2004779, BRAWH2004842,

BRAWH2004884, BRAWH2004923, BRAWH2005008, BRAWH2005074, BRAWH2005225, BRAWH2005315, BRAWH2005517, BRAWH2005524, BRAWH2005533, BRAWH2005578, BRAWH2005661, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2006996, BRAWH2007069, BRAWH2007308, BRAWH2007406, BRAWH2007550, BRAWH2007570, BRAWH2007591, BRAWH2007605, BRAWH2007658, BRAWH2007664, BRAWH2007808, BRAWH2007825, BRAWH2007862, BRAWH2007890, BRAWH2008058, BRAWH2008128, BRAWH2008219, BRAWH2008255, BRAWH2008292, BRAWH2008297, BRAWH2008366, BRAWH2008380,

BRAWH2008540, BRAWH2008577, BRAWH2008706, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009304, BRAWH2009307, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010536, BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400,

BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014234, BRAWH2014379, BRAWH2014414, BRAWH2014473, BRAWH2014645, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595,

BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017542, BRAWH2017635, BRAWH2017913, BRAWH2018206, BRAWH2018267, BRAWH2018282, BRAWH2018317, BRAWH2018506, BRAWH2018514, BRAWH2018526, BRAWH2018527, BRAWH2018601, BRAWH2018745, BRAWH2018875, BRAWH2019053, BRAWH2019055, BRAWH2019198,

BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002574, BRAWH3002600, BRAWH3002819, BRAWH3002853, BRAWH3003019, BRAWH3003136, BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047,

BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662,

BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016715, BRAWH3017259, BRAWH3017260, BRAWH3017477, BRAWH3017537, BRAWH3017980, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021545, BRAWH3021574, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172,

BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448,

BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN1000168, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001223,

BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003944, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894,

BRCAN2009156, BRCAN2009168, BRCAN2009203, BRCAN2009432, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566,

BRCAN2018667, BRCAN2018748, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368, BRCAN2025712, BRCAN2025982, BRCAN2026117, BRCAN2026197, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028036, BRCAN2028040, BRCAN2028319,

BRCAN2028338, BRCAN2028355, BRCAN2028378, BRCAN2028460, BRCAN2028545, BRCAN2028634, BRCAN2028702, BRCOC1000040, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003187, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164,

BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010123, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661,

BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019910, BRCOC2019934, BRCOC2020122, BRCOC2020142, BRHIP1000058, BRHIP1000072, BRHIP1000104, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000553, BRHIP2000621, BRHIP2000691, BRHIP2000819, BRHIP2000826, BRHIP2000920, BRHIP2001074, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172,

BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007305, BRHIP2007307, BRHIP2007616, BRHIP2007690, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414,

BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387, BRHIP2014954,

BRHIP2015153, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020799, BRHIP2020842,

BRHIP2020859, BRHIP2020930, BRHIP2021102, BRHIP2021289, BRHIP2021495, BRHIP2021615, BRHIP2021744, BRHIP2021762, BRHIP2021858, BRHIP2021870, BRHIP2021929, BRHIP2022006, BRHIP2022221, BRHIP2022228, BRHIP2022298, BRHIP2022326, BRHIP2022467, BRHIP2022708, BRHIP2022986, BRHIP2023071, BRHIP2023229, BRHIP2023272, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024398, BRHIP2024549, BRHIP2024742, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061,

BRHIP2026155, BRHIP2026214, BRHIP2026288, BRHIP2026346, BRHIP2026557, BRHIP2026723, BRHIP2026877, BRHIP2027017, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000017, BRHIP3000060, BRHIP3000070, BRHIP3000081, BRHIP3000111, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114,

BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007223, BRHIP3007239, BRHIP3007291,

BRHIP3007409, BRHIP3007424, BRHIP3007483, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675,

BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3018784, BRHIP3019643, BRHIP3019824, BRHIP3019880, BRHIP3019956, BRHIP3020046, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501,

BRHIP3027594, BRHIP3027651, BRHIP3027854, BRHIP3027947, BRHIP3028570, BRHIP3028742, BRSSN1000056, BRSSN1000092, BRSSN1000153, BRSSN2000097, BRSSN2000175, BRSSN2000197, BRSSN2000244, BRSSN2000293, BRSSN2000295, BRSSN2000312, BRSSN2000498, BRSSN2000518, BRSSN2000561, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133,

BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009518, BRSSN2009627, BRSSN2009630, BRSSN2009702, BRSSN2010019, BRSSN2010110, BRSSN2010587, BRSSN2010596, BRSSN2010830, BRSSN2011262, BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874,

BRSSN2014112, BRSSN2014294, BRSSN2014299, BRSSN2014424, BRSSN2014513, BRSSN2014556, BRSSN2014573, BRSSN2014610, BRSSN2014636, BRSSN2014685, BRSSN2015199, BRSSN2015238, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000083, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532,

BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012174, BRSTN2012284, BRSTN2012320, BRSTN2012380, BRSTN2012451, BRSTN2013171, BRSTN2013354, BRSTN2013502, BRSTN2013931, BRSTN2014490, BRSTN2014633, BRSTN2014751,

BRSTN2014770, BRSTN2015015, BRSTN2015889, BRSTN2015978, BRSTN2016066, BRSTN2016335, BRSTN2016678, BRSTN2016918, BRSTN2016992, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726,

BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005864, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2008316, BRTHA2008335, BRTHA2008502, BRTHA2008513, BRTHA2008527, BRTHA2008535, BRTHA2008598, BRTHA2008669, BRTHA2008955,

BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562, BRTHA2012714, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663,

BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016577, BRTHA2016765, BRTHA2016918, BRTHA2017178, BRTHA2017353, BRTHA2017364, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781,

BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027229, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252,

BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003225, BRTHA3003339, BRTHA3003343, BRTHA3003417, BRTHA3003449, BRTHA3003474, BRTHA3003490, BRTHA3003704, BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310,

BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011194, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623,

BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3021971, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000314, CD34C3000424, CD3.4C3000494, CERVX1000042, CERVX2000812, CERVX2000968, CERVX2002006, CERVX2002013, CERVX2002430, CERVX2002770,

CHONS1000058, CHONS2000172, CHONS2000797, CHONS2001287, CHONS2001382, CHONS2001797, CHONS2001834, CHONS2002419, CHONS2002829, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000009, CTONG1000010, CTONG1000020, CTONG1000022, CTONG1000040, CTONG1000052, CTONG1000056, CTONG1000062,

CTONG1000086, CTONG1000088, CTONG1000093, CTONG1000094, CTONG1000097, CTONG1000113, CTONG1000137, CTONG1000165, CTONG1000175, CTONG1000180, CTONG1000181, CTONG1000220, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042, CTONG2000063, CTONG2000201, CTONG2000218, CTONG2000279, CTONG2000303, CTONG2000330, CTONG2000395, CTONG2000411, CTONG2000452, CTONG2000465, CTONG2000469, CTONG2000480, CTONG2000561, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001366,

CTONG2001413, CTONG2001428, CTONG2001513, CTONG2001524, CTONG2001533, CTONG2001598, CTONG2001681, CTONG2001724, CTONG2001748, CTONG2001749, CTONG2001800, CTONG2001820, CTONG2001858, CTONG2001877, CTONG2001911, CTONG2001932, CTONG2001955, CTONG2002066, CTONG2002095, CTONG2002143, CTONG2002270, CTONG2002395, CTONG2002417, CTONG2002418, CTONG2002453, CTONG2002520, CTONG2002558, CTONG2002590, CTONG2002709, CTONG2002721, CTONG2002744, CTONG2002766, CTONG2002816, CTONG2002820, CTONG2002832, CTONG2002841, CTONG2002903, CTONG2002965, CTONG2002999, CTONG2003028, CTONG2003094, CTONG2003100, CTONG2003115, CTONG2003189, CTONG2003245, CTONG2003293, CTONG2003298, CTONG2003348, CTONG2003350, CTONG2003361,

CTONG2003372, CTONG2003375, CTONG2003427, CTONG2003524, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004423, CTONG2004454, CTONG2004550, CTONG2004669, CTONG2004716, CTONG2004912, CTONG2004941, CTONG2004948, CTONG2005049, CTONG2005110, CTONG2005145, CTONG2005265, CTONG2005278, CTONG2005290, CTONG2005374, CTONG2005399, CTONG2005468, CTONG2005567, CTONG2005615, CTONG2005635, CTONG2005775, CTONG2005795, CTONG2005890, CTONG2005904, CTONG2005913, CTONG2006129, CTONG2006223, CTONG2006273, CTONG2006310, CTONG2006377, CTONG2006393,

CTONG2006412, CTONG2006515, CTONG2006524, CTONG2006562, CTONG2006568, CTONG2006620, CTONG2006666, CTONG2006691, CTONG2006709, CTONG2006798, CTONG2006836, CTONG2006942, CTONG2006964, CTONG2007007, CTONG2007009, CTONG2007072, CTONG2007078, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007500, CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008269, CTONG2008321, CTONG2008343, CTONG2008402, CTONG2008405, CTONG2008466, CTONG2008506,

CTONG2008518, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133,

CTONG2010148, CTONG2010276, CTONG2010330, CTONG2010408, CTONG2010508, CTONG2010566, CTONG2010623, CTONG2010649, CTONG2010652, CTONG2010803, CTONG2010821, CTONG2011188, CTONG2011429, CTONG2011474, CTONG2011676, CTONG2011716, CTONG2011765, CTONG2011779, CTONG2011801, CTONG2011815, CTONG2011822, CTONG2011825, CTONG2011920, CTONG2011985, CTONG2012001, CTONG2012029, CTONG2012050, CTONG2012077, CTONG2012101, CTONG2012123, CTONG2012230, CTONG2012422, CTONG2012425, CTONG2012447, CTONG2012453, CTONG2012473, CTONG2012476, CTONG2012533, CTONG2012554, CTONG2012564, CTONG2012607, CTONG2012661, CTONG2012724, CTONG2012738, CTONG2012745, CTONG2012758, CTONG2012843, CTONG2012847, CTONG2012901, CTONG2012996,

CTONG2013128, CTONG2013149, CTONG2013156, CTONG2013178, CTONG2013222, CTONG2013284, CTONG2013339, CTONG2013348, CTONG2013352, CTONG2013381, CTONG2013393, CTONG2013511, CTONG2013630, CTONG2013803, CTONG2013934, CTONG2013986, CTONG2014032, CTONG2014058, CTONG2014119, CTONG2014165, CTONG2014191, CTONG2014206, CTONG2014234, CTONG2014264, CTONG2014280, CTONG2014369, CTONG2014389, CTONG2014491, CTONG2014630, CTONG2014635, CTONG2014646, CTONG2014705, CTONG2014898, CTONG2014946, CTONG2014954, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717,

CTONG2015804, CTONG2015815, CTONG2015866, CTONG2015914, CTONG2015953, CTONG2016056, CTONG2016185, CTONG2016217, CTONG2016355, CTONG2016408, CTONG2016499, CTONG2016505, CTONG2016559, CTONG2016658, CTONG2016775, CTONG2016797, CTONG2016824, CTONG2016846, CTONG2016869, CTONG2016904, CTONG2016931, CTONG2016942, CTONG2016953, CTONG2017021, CTONG2017094, CTONG2017292, CTONG2017409, CTONG2017429, CTONG2017444, CTONG2017458, CTONG2017604, CTONG2017650, CTONG2017798, CTONG2017804, CTONG2017814, CTONG2017910, CTONG2017939, CTONG2017989, CTONG2018062, CTONG2018069, CTONG2018147, CTONG2018211, CTONG2018217, CTONG2018279, CTONG2018334, CTONG2018343, CTONG2018379, CTONG2018383, CTONG2018413, CTONG2018483,

CTONG2018629, CTONG2018632, CTONG2018637, CTONG2018652, CTONG2018653, CTONG2018655, CTONG2018735, CTONG2018739, CTONG2018808, CTONG2018843, CTONG2018898, CTONG2018900, CTONG2018976, CTONG2019029, CTONG2019063, CTONG2019144, CTONG2019232, CTONG2019248, CTONG2019400, CTONG2019652, CTONG2019704, CTONG2019788, CTONG2019833, CTONG2020031, CTONG2020108, CTONG2020127, CTONG2020171, CTONG2020374, CTONG2020378, CTONG2020411, CTONG2020445, CTONG2020522, CTONG2020560, CTONG2020582, CTONG2020638, CTONG2020806, CTONG2020831, CTONG2020974, CTONG2021024, CTONG2021132, CTONG2021168, CTONG2021289, CTONG2022153, CTONG2022601, CTONG2023021, CTONG2023449, CTONG2023512, CTONG2024031, CTONG2024206, CTONG2024614,

CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028124, CTONG2028208, CTONG2028309, CTONG2028486, CTONG2028518, CTONG2028687, CTONG2028758, CTONG3000657, CTONG3000686, CTONG3000707, CTONG3000896, CTONG3001123, CTONG3001420, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002674, CTONG3002728, CTONG3002835, CTONG3002909, CTONG3002947,

CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009028, CTONG3009227, CTONG3009239, CTONG3009287,

CTONG3009328, CTONG3009385, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001670, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000358, D6OST2000464, D6OST2000507, D9OST2000031, D9OST2000278, D9OST2000440, D9OST2000869, D9OST2001319, D9OST2001433, D9OST2002397, D9OST2002608, D9OST2002673, D9OST2003106,

D9OST2003108, D9OST2003137, D9OST2003397, D9OST2003580, D9OST2003594, D9OST2003762, D9OST2003791, D9OST2003989, D9OST2004018, D9OST2004073, D9OST2004417, DFNES1000003, DFNES1000083, DFNES1000107, DFNES1000185, DFNES2000143, DFNES2000146, DFNES2000153, DFNES2000212, DFNES2000426, DFNES2000432, DFNES2000443, DFNES2000457, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2002966, DFNES2003081, DFNES2003192, DFNES2003255, DFNES2003742, DFNES2004346, DFNES2004354, DFNES2004371, DFNES2004383,

DFNES2004608, DFNES2004684, DFNES2005266, DFNES2005527, DFNES2005540, DFNES2005690, DFNES2005766, DFNES2005779, DFNES2005875, DFNES2006346, DFNES2006944, DFNES2007113, DFNES2007332, DFNES2007634, DFNES2007641, DFNES2007757, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000023, FCBBF1000024, FCBBF1000038, FCBBF1000053, FCBBF1000061, FCBBF1000063, FCBBF1000069, FCBBF1000115, FCBBF1000117, FCBBF1000118, FCBBF1000121, FCBBF1000125, FCBBF1000131, FCBBF1000139, FCBBF1000155, FCBBF1000157, FCBBF1000171, FCBBF1000174, FCBBF1000182,

FCBBF1000198, FCBBF1000206, FCBBF1000220, FCBBF1000232, FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000403, FCBBF1000412, FCBBF1000425, FCBBF1000437, FCBBF1000449, FCBBF1000476, FCBBF1000506, FCBBF1000509, FCBBF1000546, FCBBF1000550, FCBBF1000556, FCBBF1000563, FCBBF1000574, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF1000760, FCBBF2000038, FCBBF2000087, FCBBF2000094,

FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566, FCBBF2000576, FCBBF2000677, FCBBF2000678, FCBBF2000685, FCBBF2000733, FCBBF2000782, FCBBF2000808, FCBBF2000815, FCBBF2000825, FCBBF2000885, FCBBF2000940, FCBBF2000951, FCBBF2001001, FCBBF2001088, FCBBF2001105, FCBBF2001116, FCBBF2001183, FCBBF2001188, FCBBF2001211, FCBBF2001238, FCBBF2001243, FCBBF2001291, FCBBF2001299, FCBBF2001309, FCBBF2001400, FCBBF2001427, FCBBF2001480, FCBBF2001529, FCBBF2001594, FCBBF2001672, FCBBF2001675, FCBBF2001718, FCBBF2001720, FCBBF2001817, FCBBF2001868, FCBBF2002044,

FCBBF2002111, FCBBF2002281, FCBBF2002349, FCBBF2002370, FCBBF2002541, FCBBF2002626, FCBBF2002801, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005733, FCBBF2005909, FCBBF2005966, FCBBF2006131, FCBBF2006380, FCBBF2006418, FCBBF2006452, FCBBF2006634,

FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000110, FCBBF3000115, FCBBF3000120, FCBBF3000168, FCBBF3000175, FCBBF3000227, FCBBF3000228, FCBBF3000229, FCBBF3000233, FCBBF3000269, FCBBF3000361, FCBBF3000367, FCBBF3000388, FCBBF3000434, FCBBF3000462, FCBBF3000473, FCBBF3000501, FCBBF3000518, FCBBF3000536, FCBBF3000550, FCBBF3000847, FCBBF3001018, FCBBF3001020, FCBBF3001081, FCBBF3001235, FCBBF3001281, FCBBF3001302, FCBBF3001377, FCBBF3001470, FCBBF3001594, FCBBF3001632, FCBBF3001657, FCBBF3001818, FCBBF3001855, FCBBF3001890, FCBBF3001912, FCBBF3001914, FCBBF3001918, FCBBF3001924, FCBBF3001977,

FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004261, FCBBF3004323, FCBBF3004390, FCBBF3004409, FCBBF3004502, FCBBF3004715, FCBBF3004842, FCBBF3004847, FCBBF3004955, FCBBF3005160, FCBBF3005218, FCBBF3005320, FCBBF3005444, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007244, FCBBF3007248, FCBBF3007453,

FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008153, FCBBF3008159, FCBBF3008251, FCBBF3008311, FCBBF3008362, FCBBF3008382, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586,

FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178,

FCBBF3016451, FCBBF3016618, FCBBF3016622, FCBBF3016644, FCBBF3016667, FCBBF3016928, FCBBF3016987, FCBBF3017024, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018796, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019714, FCBBF3019716, FCBBF3019784, FCBBF3019839, FCBBF3019867, FCBBF3019961, FCBBF3020030, FCBBF3020138, FCBBF3020140, FCBBF3020163, FCBBF3020232, FCBBF3020599,

FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022765, FCBBF3022767, FCBBF3022894, FCBBF3023061, FCBBF3023078, FCBBF3023368, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026308,

FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028244, FCBBF3028472, FCBBF3028528, FCBBF3028582, FCBBF3028593, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000500, FCBBF4000529, FCBBF4000548, FCBBF5000041, FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000022,

FEBRA1000030, FEBRA1000057, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000129, FEBRA2000210, FEBRA2000220, FEBRA2000228, FEBRA2000253, FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000452, FEBRA2000454, FEBRA2000462, FEBRA2000468, FEBRA2000491, FEBRA2000504, FEBRA2000510, FEBRA2000532, FEBRA2000536, FEBRA2000538, FEBRA2000575, FEBRA2000656,

FEBRA2000659, FEBRA2000680, FEBRA2000690, FEBRA2000727, FEBRA2000734, FEBRA2000740, FEBRA2000757, FEBRA2000762, FEBRA2000771, FEBRA2000772, FEBRA2000782, FEBRA2000787, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000881, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001245, FEBRA2001267, FEBRA2001294, FEBRA2001334, FEBRA2001351, FEBRA2001383, FEBRA2001438,

FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001752, FEBRA2001772, FEBRA2001790, FEBRA2001814, FEBRA2001828, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002429, FEBRA2002442, FEBRA2002456, FEBRA2002508, FEBRA2002525, FEBRA2002552, FEBRA2002574, FEBRA2002601, FEBRA2002611, FEBRA2002628, FEBRA2002662, FEBRA2002682,

FEBRA2002707, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003100, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003750, FEBRA2003897, FEBRA2003907, FEBRA2003930, FEBRA2004023, FEBRA2004026, FEBRA2004029, FEBRA2004042, FEBRA2004053, FEBRA2004056, FEBRA2004091, FEBRA2004110,

FEBRA2004191, FEBRA2004202, FEBRA2004219, FEBRA2004237, FEBRA2004268, FEBRA2004293, FEBRA2004325, FEBRA2004329, FEBRA2004331, FEBRA2004412, FEBRA2004443, FEBRA2004485, FEBRA2004490, FEBRA2004538, FEBRA2004592, FEBRA2004620, FEBRA2004628, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005416, FEBRA2005427, FEBRA2005458, FEBRA2005476, FEBRA2005701, FEBRA2005726, FEBRA2005734, FEBRA2005778, FEBRA2005787, FEBRA2005788, FEBRA2005824, FEBRA2005995, FEBRA2005998, FEBRA2006055,

FEBRA2006061, FEBRA2006092, FEBRA2006112, FEBRA2006150, FEBRA2006165, FEBRA2006255, FEBRA2006315, FEBRA2006346, FEBRA2006354, FEBRA2006357, FEBRA2006368, FEBRA2006370, FEBRA2006372, FEBRA2006379, FEBRA2006396, FEBRA2006401, FEBRA2006409, FEBRA2006418, FEBRA2006427, FEBRA2006476, FEBRA2006491, FEBRA2006519, FEBRA2006521, FEBRA2006553, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566,

FEBRA2007589, FEBRA2007613, FEBRA2007620, FEBRA2007622, FEBRA2007708, FEBRA2007714, FEBRA2007793, FEBRA2007818, FEBRA2007823, FEBRA2007880, FEBRA2007900, FEBRA2007901, FEBRA2007931, FEBRA2007957, FEBRA2008009, FEBRA2008036, FEBRA2008086, FEBRA2008087, FEBRA2008134, FEBRA2008135, FEBRA2008159, FEBRA2008165, FEBRA2008172, FEBRA2008201, FEBRA2008210, FEBRA2008255, FEBRA2008265, FEBRA2008266, FEBRA2008282, FEBRA2008287, FEBRA2008311, FEBRA2008341, FEBRA2008360, FEBRA2008459, FEBRA2008468, FEBRA2008475, FEBRA2008481, FEBRA2008542, FEBRA2008660, FEBRA2008662, FEBRA2008681, FEBRA2008692, FEBRA2008741, FEBRA2008755, FEBRA2008836, FEBRA2008859, FEBRA2008861, FEBRA2008866, FEBRA2008881, FEBRA2008928,

FEBRA2008983, FEBRA2009016, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009419, FEBRA2009437, FEBRA2009478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397,

FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014417, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433,

FEBRA2018476, FEBRA2018746, FEBRA2019172, FEBRA2019242, FEBRA2019389, FEBRA2019457, FEBRA2019519, FEBRA2019582, FEBRA2019637, FEBRA2019663, FEBRA2019690, FEBRA2019711, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022055, FEBRA2022148, FEBRA2022204, FEBRA2022255, FEBRA2022322, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285,

FEBRA2023351, FEBRA2023377, FEBRA2023399, FEBRA2023498, FEBRA2023550, FEBRA2023721, FEBRA2023927, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025427, FEBRA2025463, FEBRA2025477, FEBRA2025838, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEBRA2028516, FEHRT2000325, FEHRT2000364, FEKID1000018, FELIV1000101, FELIV1000137, FELIV1000164,

FELNG2000241, HCASM1000021, HCASM1000050, HCASM1000061, HCASM1000115, HCASM1000130, HCASM2000016, HCASM2000138, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000131, HCHON1000142,

HCHON1000166, HCHON1000176, HCHON2000038, HCHON2000062, HCHON2000087, HCHON2000100, HCHON2000156, HCHON2000160, HCHON2000199, HCHON2000212, HCHON2000265, HCHON2000271, HCHON2000315, HCHON2000322, HCHON2000323, HCHON2000344, HCHON2000395, HCHON2000417, HCHON2000418, HCHON2000473, HCHON2000475, HCHON2000503, HCHON2000626, HCHON2000660, HCHON2000663, HCHON2000673, HCHON2000698, HCHON2000699, HCHON2000738, HCHON2000743, HCHON2000815, HCHON2000817, HCHON2000818, HCHON2000826, HCHON2000832, HCHON2000851, HCHON2000898, HCHON2000935, HCHON2000942, HCHON2000956, HCHON2000994, HCHON2001039, HCHON2001050, HCHON2001084, HCHON2001099, HCHON2001110, HCHON2001116, HCHON2001161, HCHON2001214, HCHON2001233,

HCHON2001269, HCHON2001359, HCHON2001407, HCHON2001434, HCHON2001450, HCHON2001453, HCHON2001497, HCHON2001505, HCHON2001519, HCHON2001523, HCHON2001535, HCHON2001546, HCHON2001548, HCHON2001577, HCHON2001604, HCHON2001607, HCHON2001646, HCHON2001665, HCHON2001712, HCHON2001768, HCHON2001801, HCHON2001853, HCHON2002123, HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004002, HCHON2004007, HCHON2004279, HCHON2004359, HCHON2004531, HCHON2004776, HCHON2004858, HCHON2005048, HCHON2005118, HCHON2005166,

HCHON2005802, HCHON2005921, HCHON2005987, HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HCHON2010543, HEART1000010, HEART1000074, HEART1000088, HEART1000099, HEART1000102, HEART1000118, HEART1000132, HEART1000139, HEART1000142, HEART1000149, HEART1000151, HEART1000173, HEART1000185, HEART2000024, HEART2000035, HEART2000099, HEART2000298, HEART2000306,

HEART2000309, HEART2000411, HEART2000418, HEART2000444, HEART2000448, HEART2000492, HEART2000506, HEART2000520, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006521, HEART2006557, HEART2006787, HEART2006789, HEART2006909, HEART2007031, HEART2007231, HEART2007443,

HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC1000114, HHDPC1000118, HHDPC1000163, HHDPC2000055, HHDPC2000070, HHDPC2000102, HHDPC2000104, HHDPC2000115, HHDPC2000149, HHDPC2000258, HHDPC2000315, HHDPC2000456, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006862, HHDPC2006927, HHDPC2007267,

HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030, HLUNG1000034, HLUNG1000037, HLUNG1000053, HLUNG1000055, HLUNG1000076, HLUNG1000084, HLUNG1000091, HLUNG1000099, HLUNG1000104, HLUNG1000105, HLUNG1000110, HLUNG1000151, HLUNG1000169, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363,

HLUNG2000412, HLUNG2000431, HLUNG2000480, HLUNG2000489, HLUNG2000501, HLUNG2000549, HLUNG2000557, HLUNG2000570, HLUNG2000751, HLUNG2000761, HLUNG2000777, HLUNG2000846, HLUNG2000870, HLUNG2000884, HLUNG2000926, HLUNG2000950, HLUNG2000955, HLUNG2000970, HLUNG2001013, HLUNG2001126, HLUNG2001144, HLUNG2001146, HLUNG2001214, HLUNG2001439, HLUNG2001459, HLUNG2001633, HLUNG2001677, HLUNG2001712, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002465, HLUNG2002562, HLUNG2002648, HLUNG2002659, HLUNG2002707, HLUNG2002811, HLUNG2002916, HLUNG2002942, HLUNG2002949, HLUNG2002958,

HLUNG2002982, HLUNG2003003, HLUNG2003042, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004037, HLUNG2004067, HLUNG2004159, HLUNG2004170, HLUNG2004217, HLUNG2004273, HLUNG2004416, HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026,

HLUNG2006067, HLUNG2006094, HLUNG2006370, HLUNG2006397, HLUNG2006570, HLUNG2006599, HLUNG2006614, HLUNG2006671, HLUNG2006781, HLUNG2006812, HLUNG2006843, HLUNG2006852, HLUNG2006935, HLUNG2007041, HLUNG2007163, HLUNG2007219, HLUNG2007245, HLUNG2007433, HLUNG2007931, HLUNG2008066, HLUNG2008139, HLUNG2008153, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008637, HLUNG2008833, HLUNG2008875, HLUNG2008885, HLUNG2008958, HLUNG2008959, HLUNG2009067, HLUNG2009215, HLUNG2009225, HLUNG2009253, HLUNG2009303, HLUNG2009413, HLUNG2009729, HLUNG2010181, HLUNG2010464, HLUNG2010562, HLUNG2010845,

HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017262, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA1000137, HSYRA1000148, HSYRA1000178, HSYRA2000044, HSYRA2000135, HSYRA2000137, HSYRA2000190, HSYRA2000221, HSYRA2000224, HSYRA2000232,

HSYRA2000237, HSYRA2000248, HSYRA2000253, HSYRA2000371, HSYRA2000424, HSYRA2000507, HSYRA2000510, HSYRA2000605, HSYRA2000629, HSYRA2000640, HSYRA2000706, HSYRA2000743, HSYRA2000760, HSYRA2000787, HSYRA2000792, HSYRA2000828, HSYRA2000832, HSYRA2000927, HSYRA2001003, HSYRA2001105, HSYRA2001142, HSYRA2001153, HSYRA2001232, HSYRA2001255, HSYRA2001332, HSYRA2001353, HSYRA2001396, HSYRA2001420, HSYRA2001484, HSYRA2001552, HSYRA2001567, HSYRA2001580, HSYRA2001595, HSYRA2001631, HSYRA2001638, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007338, HSYRA2007650,

HSYRA2008154, HSYRA2008714, HSYRA2009102, IMR321000155, IMR321000158, IMR321000161, IMR321000165, IMR321000193, IMR321000207, IMR321000210, IMR321000230, IMR321000242, IMR321000266, IMR322000010, IMR322000107, IMR322000127, IMR322000144, IMR322000152, IMR322000164, IMR322000178, IMR322000223, IMR322000302, IMR322000316, IMR322000374, IMR322000379, IMR322000383, IMR322000418, IMR322000518, IMR322000555, IMR322000604, IMR322000609, IMR322000656, IMR322000672, IMR322000730, IMR322000742, IMR322000775, IMR322000791, IMR322000811, IMR322000819, IMR322000838, IMR322000859, IMR322000863, IMR322000917, IMR322000919, IMR322000953, IMR322000984, IMR322001018, IMR322001049, IMR322001167, IMR322001185,

IMR322001218, IMR322001245, IMR322001317, IMR322001318, IMR322001322, IMR322001332, IMR322001380, IMR322001438, IMR322001452, IMR322001491, IMR322001534, IMR322001541, IMR322001665, IMR322001670, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322013053, IMR322013396, IMR322013731, IMR322013805, IMR322015523, IMR322016146, IMR322017049, IMR322018117, IMR322018192, IMR322019070,

JCMLC2000273, KIDNE1000008, KIDNE1000012, KIDNE1000015, KIDNE1000028, KIDNE1000036, KIDNE1000064, KIDNE1000104, KIDNE1000121, KIDNE1000143, KIDNE1000145, KIDNE1000152, KIDNE2000026, KIDNE2000041, KIDNE2000046, KIDNE2000051, KIDNE2000070, KIDNE2000085, KIDNE2000167, KIDNE2000174, KIDNE2000192, KIDNE2000227, KIDNE2000244, KIDNE2000245, KIDNE2000266, KIDNE2000287, KIDNE2000315, KIDNE2000317, KIDNE2000328, KIDNE2000330, KIDNE2000335, KIDNE2000349, KIDNE2000375, KIDNE2000383, KIDNE2000394, KIDNE2000403, KIDNE2000422, KIDNE2000493, KIDNE2000497, KIDNE2000510, KIDNE2000513, KIDNE2000517, KIDNE2000519, KIDNE2000555, KIDNE2000624, KIDNE2000665, KIDNE2000678, KIDNE2000717, KIDNE2000721, KIDNE2000722,

KIDNE2000777, KIDNE2000801, KIDNE2000832, KIDNE2000833, KIDNE2000846, KIDNE2000867, KIDNE2000909, KIDNE2000947, KIDNE2001117, KIDNE2001140, KIDNE2001162, KIDNE2001269, KIDNE2001361, KIDNE2001373, KIDNE2001636, KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002262, KIDNE2002438, KIDNE2002483, KIDNE2002725, KIDNE2002768, KIDNE2002795, KIDNE2002798, KIDNE2002839, KIDNE2002845, KIDNE2002872, KIDNE2002883, KIDNE2002980, KIDNE2002991, KIDNE2003185, KIDNE2003188, KIDNE2003233, KIDNE2003305, KIDNE2003316, KIDNE2003335, KIDNE2003357, KIDNE2003373,

KIDNE2003377, KIDNE2003507, KIDNE2003752, KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411, KIDNE2004475, KIDNE2004519, KIDNE2004520, KIDNE2004531, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629,

KIDNE2005676, KIDNE2005781, KIDNE2005798, KIDNE2005854, KIDNE2005908, KIDNE2005937, KIDNE2006009, KIDNE2006014, KIDNE2006030, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006465, KIDNE2006580, KIDNE2006652, KIDNE2006687, KIDNE2006733, KIDNE2006760, KIDNE2006775, KIDNE2006811, KIDNE2006820, KIDNE2007005, KIDNE2007040, KIDNE2007077, KIDNE2007186, KIDNE2007222, KIDNE2007328, KIDNE2007352, KIDNE2007356, KIDNE2007422, KIDNE2007569, KIDNE2007810, KIDNE2007811, KIDNE2007910, KIDNE2007944, KIDNE2007954, KIDNE2008022, KIDNE2008069, KIDNE2008072, KIDNE2008117, KIDNE2008218,

KIDNE2008315, KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009367, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750,

KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012440, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298,

KIDNE2014320, KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539, KIDNE2016570, KIDNE2016918, KIDNE2017007, KIDNE2017040, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071,

KIDNE2018166, KIDNE2018167, KIDNE2018217, KIDNE2018254, KIDNE2018268, KIDNE2018269, KIDNE2018287, KIDNE2018294, KIDNE2018352, KIDNE2018462, KIDNE2018493, KIDNE2018617, KIDNE2018678, KIDNE2018727, KIDNE2018863, KIDNE2018891, KIDNE2018989, KIDNE2018996, KIDNE2019074, KIDNE2019187, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062,

NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000091, NOVAR1000102, NOVAR2000038, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000014, NT2NE1000018, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000174, NT2NE2000195, NT2NE2000222, NT2NE2000259, NT2NE2000284, NT2NE2000299,

NT2NE2000327, NT2NE2000353, NT2NE2000356, NT2NE2000369, NT2NE2000374, NT2NE2000383, NT2NE2000384, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000575, NT2NE2000586, NT2NE2000612, NT2NE2000636, NT2NE2000658, NT2NE2000706, NT2NE2000707, NT2NE2000763, NT2NE2000802, NT2NE2000808, NT2NE2000809, NT2NE2000864, NT2NE2000909, NT2NE2000950, NT2NE2000951, NT2NE2000963, NT2NE2000980, NT2NE2001005, NT2NE2001021, NT2NE2001040, NT2NE2001048, NT2NE2001049, NT2NE2001142, NT2NE2001156, NT2NE2001176, NT2NE2001180, NT2NE2001181, NT2NE2001188, NT2NE2001191, NT2NE2001247, NT2NE2001252, NT2NE2001324,

NT2NE2001326, NT2NE2001337, NT2NE2001364, NT2NE2001372, NT2NE2001397, NT2NE2001403, NT2NE2001428, NT2NE2001432, NT2NE2001435, NT2NE2001442, NT2NE2001524, NT2NE2001530, NT2NE2001545, NT2NE2001598, NT2NE2001623, NT2NE2001626, NT2NE2001634, NT2NE2001648, NT2NE2001655, NT2NE2001660, NT2NE2001666, NT2NE2001677, NT2NE2001697, NT2NE2001698, NT2NE2001723, NT2NE2001725, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003185, NT2NE2003252, NT2NE2003280, NT2NE2003308, NT2NE2003309, NT2NE2003315, NT2NE2003393, NT2NE2003408,

NT2NE2003457, NT2NE2003485, NT2NE2003540, NT2NE2003569, NT2NE2003571, NT2NE2003705, NT2NE2003752, NT2NE2003921, NT2NE2004255, NT2NE2004378, NT2NE2004490, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005419, NT2NE2005441, NT2NE2005517, NT2NE2005537, NT2NE2005555, NT2NE2005588, NT2NE2005676, NT2NE2005720, NT2NE2005784, NT2NE2005804, NT2NE2005821, NT2NE2005876, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006458, NT2NE2006478,

NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010854, NT2NE2011033, NT2NE2011036, NT2NE2011107,

NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511,

NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018594, NT2NE2018739, NT2NE2018916, NT2NE2019056, NT2NE2019076, NT2NE2019091, NT2NE2019099, NT2RI1000016, NT2RI1000035, NT2RI1000048, NT2RI1000127, NT2RI1000164, NT2RI1000172, NT2RI2000004, NT2RI2000007,

NT2RI2000107, NT2RI2000116, NT2RI2000117, NT2RI2000128, NT2RI2000133, NT2RI2000167, NT2RI2000173, NT2RI2000187, NT2RI2000263, NT2RI2000270, NT2RI2000276, NT2RI2000294, NT2RI2000313, NT2RI2000341, NT2RI2000348, NT2RI2000412, NT2RI2000421, NT2RI2000480, NT2RI2000575, NT2RI2000588, NT2RI2000597, NT2RI2000669, NT2RI2000685, NT2RI2000688, NT2RI2000689, NT2RI2000704, NT2RI2000738, NT2RI2000775, NT2RI2000822, NT2RI2000829, NT2RI2000841, NT2RI2000932, NT2RI2000974, NT2RI2000987, NT2RI2001010, NT2RI2001017, NT2RI2001030, NT2RI2001083, NT2RI2001091, NT2RI2001167, NT2RI2001178, NT2RI2001230, NT2RI2001235, NT2RI2001244, NT2RI2001307, NT2RI2001342, NT2RI2001385, NT2RI2001409, NT2RI2001410, NT2RI2001422,

NT2RI2001425, NT2RI2001449, NT2RI2001450, NT2RI2001474, NT2RI2001519, NT2RI2001595, NT2RI2001621, NT2RI2001624, NT2RI2001726, NT2RI2001782, NT2RI2001836, NT2RI2001866, NT2RI2001910, NT2RI2001952, NT2RI2002022, NT2RI2002041, NT2RI2002117, NT2RI2002120, NT2RI2002153, NT2RI2002179, NT2RI2002243, NT2RI2002252, NT2RI2002260, NT2RI2002266, NT2RI2002270, NT2RI2002334, NT2RI2002359, NT2RI2002391, NT2RI2002447, NT2RI2002481, NT2RI2002507, NT2RI2002517, NT2RI2002540, NT2RI2002549, NT2RI2002554, NT2RI2002564, NT2RI2002585, NT2RI2002592, NT2RI2002602, NT2RI2002699, NT2RI2002729, NT2RI2002802, NT2RI2002819, NT2RI2002847, NT2RI2002852, NT2RI2002865, NT2RI2002923, NT2RI2002926, NT2RI2002958, NT2RI2002970,

NT2RI2003011, NT2RI2003019, NT2RI2003067, NT2RI2003184, NT2RI2003205, NT2RI2003222, NT2RI2003301, NT2RI2003317, NT2RI2003338, NT2RI2003344, NT2RI2003360, NT2RI2003361, NT2RI2003383, NT2RI2003392, NT2RI2003399, NT2RI2003419, NT2RI2003420, NT2RI2003481, NT2RI2003487, NT2RI2003556, NT2RI2003655, NT2RI2003667, NT2RI2003678, NT2RI2003695, NT2RI2003884, NT2RI2003921, NT2RI2003973, NT2RI2003993, NT2RI2004059, NT2RI2004078, NT2RI2004079, NT2RI2004093, NT2RI2004136, NT2RI2004157, NT2RI2004190, NT2RI2004209, NT2RI2004230, NT2RI2004290, NT2RI2004304, NT2RI2004312, NT2RI2004381, NT2RI2004398, NT2RI2004410, NT2RI2004442, NT2RI2004468, NT2RI2004535, NT2RI2004606, NT2RI2004608, NT2RI2004618, NT2RI2004783,

NT2RI2004818, NT2RI2004840, NT2RI2004916, NT2RI2004962, NT2RI2004984, NT2RI2004985, NT2RI2005048, NT2RI2005061, NT2RI2005069, NT2RI2005087, NT2RI2005096, NT2RI2005130, NT2RI2005150, NT2RI2005166, NT2RI2005239, NT2RI2005246, NT2RI2005315, NT2RI2005353, NT2RI2005358, NT2RI2005368, NT2RI2005382, NT2RI2005473, NT2RI2005520, NT2RI2005564, NT2RI2005621, NT2RI2005628, NT2RI2005647, NT2RI2005670, NT2RI2005713, NT2RI2005723, NT2RI2005772, NT2RI2005811, NT2RI2005816, NT2RI2005818, NT2RI2005851, NT2RI2005966, NT2RI2006070, NT2RI2006071, NT2RI2006072, NT2RI2006081, NT2RI2006126, NT2RI2006127, NT2RI2006183, NT2RI2006257, NT2RI2006271, NT2RI2006294, NT2RI2006345, NT2RI2006403, NT2RI2006445, NT2RI2006487,

NT2RI2006506, NT2RI2006553, NT2RI2006565, NT2RI2006640, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006825, NT2RI2006838, NT2RI2006853, NT2RI2006855, NT2RI2006856, NT2RI2006943, NT2RI2006973, NT2RI2007046, NT2RI2007048, NT2RI2007054, NT2RI2007084, NT2RI2007096, NT2RI2007116, NT2RI2007148, NT2RI2007214, NT2RI2007303, NT2RI2007334, NT2RI2007377, NT2RI2007386, NT2RI2007406, NT2RI2007439, NT2RI2007445, NT2RI2007468, NT2RI2007469, NT2RI2007507, NT2RI2007589, NT2RI2007593, NT2RI2007629, NT2RI2007723, NT2RI2007729, NT2RI2007751, NT2RI2007754, NT2RI2007877, NT2RI2007879, NT2RI2007884,

NT2RI2007891, NT2RI2007956, NT2RI2007965, NT2RI2007987, NT2RI2008007, NT2RI2008050, NT2RI2008171, NT2RI2008188, NT2RI2008204, NT2RI2008221, NT2RI2008233, NT2RI2008396, NT2RI2008455, NT2RI2008481, NT2RI2008502, NT2RI2008566, NT2RI2008598, NT2RI2008622, NT2RI2008656, NT2RI2008714, NT2RI2008749, NT2RI2008801, NT2RI200880.8, NT2RI2008812, NT2RI2008841, NT2RI2008942, NT2RI2008952, NT2RI2009005, NT2RI2009037, NT2RI2009066, NT2RI2009083, NT2RI2009101, NT2RI2009103, NT2RI2009144, NT2RI2009151, NT2RI2009173, NT2RI2009194, NT2RI2009215, NT2RI2009239, NT2RI2009259, NT2RI2009269, NT2RI2009281, NT2RI2009289, NT2RI2009301, NT2RI2009402, NT2RI2009406, NT2RI2009517, NT2RI2009585, NT2RI2009595, NT2RI2009855,

NT2RI2010283, NT2RI2010508, NT2RI2010795, NT2RI2011422, NT2RI2011450, NT2RI2011683, NT2RI2011803, NT2RI2012659, NT2RI2012726, NT2RI2012990, NT2RI2013345, NT2RI2013357, NT2RI2013373, NT2RI2014247, NT2RI2014551, NT2RI2014683, NT2RI2014733, NT2RI2015239, NT2RI2015342, NT2RI2015533, NT2RI2016128, NT2RI2016134, NT2RI2017529, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976,

NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171,

NT2RI3006284, NT2RI3006340, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000005, NT2RP6000008, NT2RP6000017, NT2RP6000032, NT2RP6000033, NT2RP6000039, NT2RP6000043, NT2RP6000059, NT2RP6000060, NT2RP6000078, NT2RP6000085, NT2RP6000086, NT2RP6000100, NT2RP6000109, NT2RP6000110, NT2RP6000119, NT2RP6000123, NT2RP6000127, NT2RP6000130, NT2RP6000140, NT2RP6000150, NT2RP7000041,

NT2RP7000069, NT2RP7000076, NT2RP7000086, NT2RP7000109, NT2RP7000112, NT2RP7000173, NT2RP7000259, NT2RP7000271, NT2RP7000311, NT2RP7000359, NT2RP7000364, NT2RP7000380, NT2RP7000391, NT2RP7000425, NT2RP7000477, NT2RP7000579, NT2RP7000586, NT2RP7000600, NT2RP7000812, NT2RP7000906, NT2RP7000926, NT2RP7001074, NT2RP7001080, NT2RP7001156, NT2RP7001231, NT2RP7001283, NT2RP7001306, NT2RP7001319, NT2RP7001335, NT2RP7001364, NT2RP7001475, NT2RP7001532, NT2RP7001591, NT2RP7001602, NT2RP7001678, NT2RP7001856, NT2RP7001962, NT2RP7002028, NT2RP7002064, NT2RP7002151, NT2RP7002163, NT2RP7002243, NT2RP7002282, NT2RP7002376, NT2RP7002449, NT2RP7002478, NT2RP7002554, NT2RP7002594, NT2RP7002603, NT2RP7002650,

NT2RP7002738, NT2RP7002779, NT2RP7002829, NT2RP7002841, NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003203, NT2RP7003261, NT2RP7003319, NT2RP7003439, NT2RP7003511, NT2RP7003629, NT2RP7003632, NT2RP7003647, NT2RP7003680, NT2RP7003688, NT2RP7003724, NT2RP7004027, NT2RP7004080, NT2RP7004196, NT2RP7004233, NT2RP7004260, NT2RP7004348, NT2RP7004352, NT2RP7004358, NT2RP7004373, NT2RP7004396, NT2RP7004428, NT2RP7004481, NT2RP7004541, NT2RP7004559, NT2RP7004641, NT2RP7004656, NT2RP7004687, NT2RP7004722, NT2RP7004751, NT2RP7004766, NT2RP7004790, NT2RP7004884, NT2RP7004911, NT2RP7004915,

NT2RP7004925, NT2RP7004946, NT2RP7004961, NT2RP7004964, NT2RP7004975, NT2RP7005118, NT2RP7005205, NT2RP7005219, NT2RP7005323, NT2RP7005468, NT2RP7005493, NT2RP7005529, NT2RP7005629, NT2RP7005631, NT2RP7005669, NT2RP7005675, NT2RP7005846, NT2RP7006033, NT2RP7006075, NT2RP7006160, NT2RP7006162, NT2RP7006223, NT2RP7006296, NT2RP7006304, NT2RP7006374, NT2RP7006395, NT2RP7006408, NT2RP7006457, NT2RP7006490, NT2RP7006539, NT2RP7006547, NT2RP7006601, NT2RP7006619, NT2RP7006621, NT2RP7006636, NT2RP7006701, NT2RP7006853, NT2RP7006886, NT2RP7006887, NT2RP7006980, NT2RP7006986, NT2RP7006995, NT2RP7007114, NT2RP7007177, NT2RP7007221, NT2RP7007226, NT2RP7007252, NT2RP7007269, NT2RP7007310, NT2RP7007359,

NT2RP7007381, NT2RP7007387, NT2RP7007406, NT2RP7007480, NT2RP7007504, NT2RP7007530, NT2RP7007537, NT2RP7007580, NT2RP7007594, NT2RP7007610, NT2RP7007617, NT2RP7007643, NT2RP7007766, NT2RP7007925, NT2RP7007930, NT2RP7008013, NT2RP7008015, NT2RP7008133, NT2RP7008137, NT2RP7008142, NT2RP7008144, NT2RP7008161, NT2RP7008167, NT2RP7008190, NT2RP7008315, NT2RP7008360, NT2RP7008396, NT2RP7008406, NT2RP7008435, NT2RP7008441, NT2RP7008454, NT2RP7008487, NT2RP7008543, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009087, NT2RP7009108, NT2RP7009147, NT2RP7009149, NT2RP7009168,

NT2RP7009215, NT2RP7009225, NT2RP7009236, NT2RP7009253, NT2RP7009267, NT2RP7009322, NT2RP7009363, NT2RP7009370, NT2RP7009373, NT2RP7009394, NT2RP7009397, NT2RP7009429, NT2RP7009439, NT2RP7009466, NT2RP7009481, NT2RP7009482, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014005,

NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000435,

NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000033, NTONG1000047, NTONG1000052, NTONG1000075, NTONG1000087, NTONG1000123, NTONG1000130, NTONG1000161, NTONG1000172, NTONG1000182, NTONG1000213, NTONG1000214, NTONG1000246, NTONG1000247, NTONG1000257, NTONG2000040, NTONG2000107, NTONG2000231, NTONG2000334, NTONG2000363, NTONG2000413, NTONG2000492, NTONG2000499, NTONG2000531, NTONG2000583, NTONG2000800, NTONG2000858, NTONG2000878,

NTONG2000966, NTONG2000985, NTONG2001014, NTONG2001079, NTONG2001157, NTONG2001173, NTONG2001220, NTONG2001222, NTONG2001362, NTONG2001428, NTONG2001532, NTONG2001550, NTONG2001587, NTONG2001612, NTONG2001619, NTONG2001642, NTONG2001657, NTONG2001676, NTONG2001762, NTONG2002278, NTONG2002582, NTONG2002807, NTONG2002948, NTONG2002970, NTONG2002985, NTONG2003158, NTONG2003210, NTONG2003316, NTONG2003409, NTONG2003454, NTONG2003515, NTONG2003805, NTONG2003852, NTONG2003897, NTONG2003928, NTONG2004095, NTONG2004308, NTONG2004521, NTONG2004614, NTONG2004690, NTONG2004806, NTONG2004829, NTONG2004918, NTONG2004991, NTONG2005062, NTONG2005086, NTONG2005137, NTONG2005153, NTONG2005265, NTONG2005277,

NTONG2005363, NTONG2005373, NTONG2005391, NTONG2005480, NTONG2005497, NTONG2005577, NTONG2005657, NTONG2005801, NTONG2005822, NTONG2005897, NTONG2005969, NTONG2006099, NTONG2006187, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008522, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030, OCBBF1000042, OCBBF1000049, OCBBF1000054,

OCBBF1000067, OCBBF1000080, OCBBF1000085, OCBBF1000086, OCBBF1000091, OCBBF1000104, OCBBF1000114, OCBBF1000118, OCBBF1000119, OCBBF1000122, OCBBF1000130, OCBBF1000138, OCBBF1000145, OCBBF1000146, OCBBF1000153, OCBBF1000175, OCBBF1000185, OCBBF1000188, OCBBF1000192, OCBBF1000254, OCBBF1000315, OCBBF2000015, OCBBF2000126, OCBBF2000178, OCBBF2000231, OCBBF2000277, OCBBF2000287, OCBBF2000323, OCBBF2000452, OCBBF2000467, OCBBF2000677, OCBBF2000703, OCBBF2000719, OCBBF2000824, OCBBF2000831, OCBBF2000904, OCBBF2000998, OCBBF2001075, OCBBF2001101, OCBBF2001124, OCBBF2001140, OCBBF2001166, OCBBF2001176, OCBBF2001186, OCBBF2001196, OCBBF2001210, OCBBF2001252, OCBBF2001307, OCBBF2001323, OCBBF2001389,

OCBBF2001402, OCBBF2001408, OCBBF2001416, OCBBF2001473, OCBBF2001492, OCBBF2001494, OCBBF2001527, OCBBF2001528, OCBBF2001586, OCBBF2001639, OCBBF2001681, OCBBF2001706, OCBBF2001749, OCBBF2001794, OCBBF2001910, OCBBF2001931, OCBBF2001961, OCBBF2001983, OCBBF2002015, OCBBF2002083, OCBBF2002086, OCBBF2002124, OCBBF2002161, OCBBF2002290, OCBBF2002357, OCBBF2002376, OCBBF2002615, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003518, OCBBF2003543, OCBBF2003593, OCBBF2003744, OCBBF2003819, OCBBF2003925, OCBBF2004038, OCBBF2004104, OCBBF2004147, OCBBF2004168, OCBBF2004235, OCBBF2004259,

OCBBF2004273, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843, OCBBF2005901, OCBBF2005956, OCBBF2006005, OCBBF2006030, OCBBF2006058, OCBBF2006113, OCBBF2006148, OCBBF2006151, OCBBF2006154, OCBBF2006172, OCBBF2006214, OCBBF2006241, OCBBF2006313, OCBBF2006388, OCBBF2006438, OCBBF2006567, OCBBF2006624, OCBBF2006639, OCBBF2006660, OCBBF2006764, OCBBF2006859,

OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007238, OCBBF2007354, OCBBF2007414, OCBBF2007415, OCBBF2007428, OCBBF2007478, OCBBF2007485, OCBBF2007520, OCBBF2007555, OCBBF2007610, OCBBF2007622, OCBBF2007756, OCBBF2007762, OCBBF2007829, OCBBF2007892, OCBBF2007931, OCBBF2007946, OCBBF2008005, OCBBF2008116, OCBBF2008138, OCBBF2008144, OCBBF2008283, OCBBF2008330, OCBBF2008340, OCBBF2008366, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770, OCBBF2008775,

OCBBF2008790, OCBBF2008814, OCBBF2009095, OCBBF2009115, OCBBF2009242, OCBBF2009301, OCBBF2009352, OCBBF2009391, OCBBF2009424, OCBBF2009536, OCBBF2009571, OCBBF2009583, OCBBF2009603, OCBBF2009772, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010830, OCBBF2010841, OCBBF2010843, OCBBF2010858, OCBBF2010863, OCBBF2010871, OCBBF2010931, OCBBF2010945, OCBBF2010978, OCBBF2010989, OCBBF2011073, OCBBF2011137, OCBBF2011160, OCBBF2011177, OCBBF2011228, OCBBF2011232, OCBBF2011283, OCBBF2011311, OCBBF2011536, OCBBF2011625,

OCBBF2011669, OCBBF2011685, OCBBF2011706, OCBBF2011722, OCBBF2011759, OCBBF2011767, OCBBF2011855, OCBBF2011872, OCBBF2011887, OCBBF2011897, OCBBF2011914, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012678, OCBBF2012704, OCBBF2012714, OCBBF2012755, OCBBF2012812, OCBBF2012821, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789, OCBBF2013843, OCBBF2013883,

OCBBF2013926, OCBBF2014052, OCBBF2014064, OCBBF2014089, OCBBF2014292, OCBBF2014322, OCBBF2014386, OCBBF2014541, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016928, OCBBF2017035, OCBBF2017055, OCBBF2017069, OCBBF2017306, OCBBF2017325, OCBBF2017398, OCBBF2017458, OCBBF2017489,

OCBBF2017516, OCBBF2017665, OCBBF2017754, OCBBF2017791, OCBBF2017815, OCBBF2017882, OCBBF2017888, OCBBF2017893, OCBBF2017920, OCBBF2018012, OCBBF2018014, OCBBF2018084, OCBBF2018167, OCBBF2018206, OCBBF2018229, OCBBF2018234, OCBBF2018362, OCBBF2018380, OCBBF2018563, OCBBF2018581, OCBBF2018618, OCBBF2018663, OCBBF2018687, OCBBF2018707, OCBBF2018827, OCBBF2018828, OCBBF2018934, OCBBF2018956, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021286, OCBBF2021323, OCBBF2021518, OCBBF2021788, OCBBF2021833,

OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708,

OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000323, OCBBF3000372,

OCBBF3000380, OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004487, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000180, PEBLM2000030,

PEBLM2000112, PEBLM2000126, PEBLM2000147, PEBLM2000170, PEBLM2000180, PEBLM2000213, PEBLM2000248, PEBLM2000270, PEBLM2000308, PEBLM2000321, PEBLM2000326, PEBLM2000333, PEBLM2000338, PEBLM2000395, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004497, PEBLM2004666, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006366, PEBLM2006709,

PEBLM2006912, PEBLM2007112, PEBLM2007140, PEBLM2007188, PEBLM2007296, PEBLM2007437, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259,

PERIC2004379, PERIC2004429, PERIC2004510, PERIC2004613, PERIC2004909, PERIC2005111, PERIC2005305, PERIC2005347, PERIC2005370, PERIC2005936, PERIC2006035, PERIC2006121, PERIC2006443, PERIC2006945, PERIC2006960, PERIC2007068, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000066, PLACE5000067, PLACE5000070, PLACE5000080, PLACE5000105, PLACE5000113, PLACE5000115, PLACE5000116, PLACE5000129, PLACE5000139, PLACE5000153, PLACE5000159, PLACE5000170, PLACE5000171, PLACE5000178, PLACE5000245, PLACE5000260, PLACE5000282, PLACE5000297, PLACE5000372, PLACE5000492, PLACE5000522,

PLACE5000527, PLACE6000012, PLACE6000044, PLACE6000055, PLACE6000070, PLACE6000137, PLACE6000145, PLACE6000191, PLACE6000205, PLACE6000231, PLACE6000263, PLACE6000296, PLACE6000314, PLACE6000325, PLACE6000348, PLACE6000371, PLACE6000414, PLACE6000424, PLACE6000426, PLACE6000429, PLACE6000450, PLACE6000463, PLACE6000523, PLACE6000550, PLACE6000552, PLACE6000555, PLACE6000619, PLACE6000630, PLACE6000953, PLACE6001064, PLACE6001118, PLACE6001153, PLACE6001185, PLACE6001262, PLACE6001281, PLACE6001294, PLACE6001443, PLACE6001536, PLACE6001557, PLACE6001712, PLACE6001740, PLACE6001823, PLACE6001886, PLACE6001923, PLACE6001925, PLACE6001933, PLACE6002011, PLACE6002016, PLACE6002056, PLACE6002102,

PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6002960, PLACE6003004, PLACE6003038, PLACE6003077, PLACE6003094, PLACE6003109, PLACE6003204, PLACE6003218, PLACE6003372, PLACE6003383, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004005, PLACE6004101, PLACE6004219, PLACE6004312, PLACE6004336, PLACE6004368, PLACE6004380, PLACE6004396, PLACE6004397, PLACE6004454, PLACE6004464, PLACE6004491, PLACE6004578, PLACE6004663, PLACE6004687, PLACE6004738,

PLACE6004931, PLACE6004966, PLACE6004993, PLACE6005007, PLACE6005029, PLACE6005108, PLACE6005231, PLACE6005234, PLACE6005283, PLACE6005294, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006042, PLACE6006077, PLACE6006137, PLACE6006158, PLACE6006186, PLACE6006217, PLACE6006222, PLACE6006266, PLACE6006287, PLACE6006394, PLACE6006418, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007436, PLACE6007482, PLACE6007687,

PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008775, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010484, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011057, PLACE6011102, PLACE6011156, PLACE6011211, PLACE6011260, PLACE6011334,

PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016254, PLACE6016383, PLACE6016942, PLACE6016984, PLACE6017002, PLACE6017431,

PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6018938, PLACE6019195, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004103, PLACE7004961, PLACE7005169,

PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000097, PROST1000110, PROST1000120, PROST1000123, PROST1000136, PROST1000145, PROST1000152, PROST1000167, PROST1000184, PROST1000215, PROST1000217, PROST1000220, PROST1000226, PROST1000246, PROST1000262, PROST1000272, PROST1000298, PROST1000322, PROST1000334, PROST1000343, PROST1000362, PROST1000377, PROST1000383, PROST1000412, PROST1000419, PROST1000451, PROST1000480,

PROST1000485, PROST1000493, PROST1000512, PROST1000527, PROST1000528, PROST1000536, PROST1000559, PROST1000575, PROST2000036, PROST2000053, PROST2000067, PROST2000079, PROST2000105, PROST2000143, PROST2000162, PROST2000165, PROST2000176, PROST2000203, PROST2000206, PROST2000212, PROST2000241, PROST2000267, PROST2000273, PROST2000274, PROST2000277, PROST2000284, PROST2000325, PROST2000337, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002, PROST2001116, PROST2001138, PROST2001180, PROST2001213, PROST2001283, PROST2001414, PROST2001415, PROST2001465, PROST2001521,

PROST2001540, PROST2001552, PROST2001599, PROST2001611, PROST2001636, PROST2001739, PROST2001796, PROST2001805, PROST2001823, PROST2001883, PROST2001899, PROST2001997, PROST2001998, PROST2002078, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003232, PROST2003302, PROST2003303, PROST2003324, PROST2003338, PROST2003340, PROST2003396, PROST2003428, PROST2003472, PROST2003537, PROST2003563,

PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004258, PROST2004332, PROST2004481, PROST2004526, PROST2004570, PROST2004727, PROST2004739, PROST2004742, PROST2004744, PROST2004817, PROST2005039, PROST2005067, PROST2005076, PROST2005093, PROST2005121, PROST2005131, PROST2005143, PROST2005228, PROST2005272, PROST2005285, PROST2005426, PROST2005466, PROST2005604, PROST2005630, PROST2005683, PROST2005788, PROST2005880, PROST2005886, PROST2005904, PROST2005919, PROST2005943, PROST2006008, PROST2006020, PROST2006030,

PROST2006060, PROST2006196, PROST2006201, PROST2006260, PROST2006282, PROST2006343, PROST2006450, PROST2006491, PROST2006510, PROST2006536, PROST2006579, PROST2006688, PROST2006737, PROST2006766, PROST2006782, PROST2006933, PROST2006988, PROST2007122, PROST2007200, PROST2007237, PROST2007248, PROST2007289, PROST2001317, PROST2007328, PROST2007382, PROST2007389, PROST2007416, PROST2007528, PROST2007612, PROST2007818, PROST2007871, PROST2007950, PROST2007956, PROST2007974, PROST2007988, PROST2008079, PROST2008088, PROST2008243, PROST2008245, PROST2008268, PROST2008271, PROST2008447, PROST2008468, PROST2008472, PROST2008489, PROST2008516, PROST2008724, PROST2008835, PROST2008993, PROST2009022,

PROST2009208, PROST2009222, PROST2009254, PROST2009273, PROST2009320, PROST2009347, PROST2009365, PROST2009388, PROST2009400, PROST2009470, PROST2009483, PROST2009571, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010579, PROST2010606, PROST2010885, PROST2011012, PROST2011038, PROST2011105, PROST2011297, PROST2011410, PROST2011439, PROST2011482, PROST2011577, PROST2011631, PROST2011660, PROST2012005, PROST2012007, PROST2012016, PROST2012088, PROST2012140, PROST2012157, PROST2012190, PROST2012249, PROST2012353, PROST2012400, PROST2012448, PROST2012504, PROST2012542,

PROST2012550, PROST2012740, PROST2012745, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014925, PROST2015124, PROST2015137, PROST2015198, PROST2015243, PROST2015246, PROST2015251, PROST2015287, PROST2015332, PROST2015457, PROST2015464, PROST2015537, PROST2015545, PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512,

PROST2016566, PROST2016582, PROST2016668, PROST2016684, PROST2016777, PROST2016829, PROST2016860, PROST2016918, PROST2016980, PROST2017098, PROST2017128, PROST2017203, PROST2017367, PROST2017413, PROST2017441, PROST2017529, PROST2017578, PROST2017612, PROST2017617, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019164, PROST2019253, PROST2019296, PROST2019398, PROST2019487, PROST2019781, PUAEN1000039, PUAEN1000057, PUAEN1000065, PUAEN1000081, PUAEN1000085, PUAEN1000087,

PUAEN1000121, PUAEN1000147, PUAEN1000161, PUAEN1000175, PUAEN1000239, PUAEN1000275, PUAEN1000276, PUAEN1000288, PUAEN2000080, PUAEN2000152, PUAEN2000247, PUAEN2000312, PUAEN2000394, PUAEN2000497, PUAEN2000535, PUAEN2000684, PUAEN2000942, PUAEN2001260, PUAEN2001345, PUAEN2001526, PUAEN2001586, PUAEN2001882, PUAEN2002473, PUAEN2002489, PUAEN2002552, PUAEN2002568, PUAEN2002616, PUAEN2002758, PUAEN2003018, PUAEN2003079, PUAEN2003116, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761,

PUAEN2007044, PUAEN2007350, PUAEN2007785, PUAEN2007898, PUAEN2008123, PUAEN2008228, PUAEN2008515, PUAEN2008815, PUAEN2008939, PUAEN2008980, PUAEN2008985, PUAEN2009174, PUAEN2009348, PUAEN2009534, PUAEN2009655, PUAEN2009795, PUAEN2009852, PUAEN2010346, PUAEN2010824, RECTM1000051, RECTM1000141, RECTM2000220, RECTM2000349, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000047, SALGL1000065, SALGL1000107, SALGL1000171, SKMUS1000014, SKMUS1000030, SKMUS1000064, SKMUS1000067, SKMUS1000084, SKMUS1000102, SKMUS1000104, SKMUS1000110, SKMUS1000118,

SKMUS1000124, SKMUS1000129, SKMUS1000138, SKMUS1000176, SKMUS1000177, SKMUS2000020, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000361, SKMUS2000365, SKMUS2000380, SKMUS2000390, SKMUS2000416, SKMUS2000458, SKMUS2000467, SKMUS2000468, SKMUS2000515, SKMUS2000690, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000873, SKMUS2000894, SKMUS2000898, SKMUS2000902, SKMUS2000933, SKMUS2000945, SKMUS2000954, SKMUS2000961, SKMUS2000973, SKMUS2000980,

SKMUS2001008, SKMUS2001014, SKMUS2001129, SKMUS2001147, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001501, SKMUS2001528, SKMUS2001543, SKMUS2001580, SKMUS2001585, SKMUS2001608, SKMUS2001631, SKMUS2001634, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034,

SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000110, SKNMC1000123, SKNMC1000124, SKNMC1000159, SKNMC1000168, SKNMC1000229, SKNMC1000251, SKNMC1000264, SKNMC2000065, SKNMC2000097, SKNMC2000224, SKNMC2000256, SKNMC2000322, SKNMC2000356, SKNMC2000374, SKNMC2000583, SKNMC2000593, SKNMC2000612, SKNMC2000622, SKNMC2000649,

SKNMC2000698, SKNMC2000966, SKNMC2001113, SKNMC2001324, SKNMC2001503, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000002, SKNSH1000086, SKNSH1000101, SKNSH1000174, SKNSH1000301, SKNSH1000308, SKNSH1000416, SKNSH2000051, SKNSH2000101, SKNSH2000151, SKNSH2000163, SKNSH2000250, SKNSH2000319, SKNSH2000347, SKNSH2000482, SKNSH2000516, SKNSH2000550, SKNSH2000716, SKNSH2000819, SKNSH2000971, SKNSH2001222, SKNSH2001875, SKNSH2001931, SKNSH2002054, SKNSH2002325, SKNSH2002768,

SKNSH2002866, SKNSH2003064, SKNSH2003174, SKNSH2003422, SKNSH2003466, SKNSH2003483, SKNSH2003528, SKNSH2003633, SKNSH2004039, SKNSH2005061, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008043, SKNSH2008969, SKNSH2009197, SKNSH2009357, SKNSH2009435, SKNSH2009991, SKNSH2010015, SMINT1000012, SMINT1000039, SMINT1000042, SMINT1000048, SMINT1000051, SMINT1000054, SMINT1000057, SMINT1000071, SMINT1000075, SMINT1000078, SMINT1000091, SMINT1000100, SMINT1000103, SMINT1000117, SMINT1000118, SMINT1000131, SMINT1000137, SMINT1000192, SMINT2000007,

SMINT2000018, SMINT2000032, SMINT2000040, SMINT2000060, SMINT2000073, SMINT2000084, SMINT2000145, SMINT2000159, SMINT2000176, SMINT2000185, SMINT2000227, SMINT2000229, SMINT2000232, SMINT2000239, SMINT2000267, SMINT2000277, SMINT2000454, SMINT2000468, SMINT2000518, SMINT2000529, SMINT2000530, SMINT2000537, SMINT2000538, SMINT2000541, SMINT2000545, SMINT2000558, SMINT2000562, SMINT2000568, SMINT2000602, SMINT2000609, SMINT2000629, SMINT2000659, SMINT2000747, SMINT2000811, SMINT2000824, SMINT2000933, SMINT2000984, SMINT2001114, SMINT2001158, SMINT2001183, SMINT2001199, SMINT2001222, SMINT2001397, SMINT2001458, SMINT2001461, SMINT2001535, SMINT2001559, SMINT2001609, SMINT2001615, SMINT2001683,

SMINT2001731, SMINT2001812, SMINT2001950, SMINT2002126, SMINT2002159, SMINT2002202, SMINT2002210, SMINT2002224, SMINT2002281, SMINT2002311, SMINT2002314, SMINT2002328, SMINT2002414, SMINT2002457, SMINT2002620, SMINT2002689, SMINT2002743, SMINT2002778, SMINT2002880, SMINT2002882, SMINT2002884, SMINT2002899, SMINT2002974, SMINT2002976, SMINT2003003, SMINT2003074, SMINT2003128, SMINT2003150, SMINT2003317, SMINT2003340, SMINT2003386, SMINT2003423, SMINT2003505, SMINT2003551, SMINT2003569, SMINT2003641, SMINT2003644, SMINT2003866, SMINT2003905, SMINT2004023, SMINT2004037, SMINT2004047, SMINT2004086, SMINT2004280, SMINT2004299, SMINT2004339, SMINT2004350, SMINT2004414, SMINT2004473, SMINT2004547,

SMINT2004583, SMINT2004589, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005368, SMINT2005387, SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796,

SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2008960, SMINT2009119, SMINT2009121, SMINT2009212, SMINT2009216, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369,

SMINT2010426, SMINT2010500, SMINT2010533, SMINT2010629, SMINT2010672, SMINT2010753, SMINT2010853, SMINT2010897, SMINT2010901, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012122, SMINT2012179, SMINT2012195, SMINT2012285, SMINT2012291, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721, SMINT2015006, SMINT2015294,

SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000134,

SPLEN1000139, SPLEN1000143, SPLEN1000144, SPLEN1000145, SPLEN1000156, SPLEN1000163, SPLEN1000165, SPLEN1000166, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000210, SPLEN2000222, SPLEN2000243, SPLEN2000247, SPLEN2000255, SPLEN2000258, SPLEN2000267, SPLEN2000270, SPLEN2000307, SPLEN2000310, SPLEN2000341, SPLEN2000348, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695,

SPLEN2000698, SPLEN2000839, SPLEN2000874, SPLEN2001135, SPLEN2001141, SPLEN2001157, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001689, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002335, SPLEN2002343, SPLEN2002354, SPLEN2002385, SPLEN2002419, SPLEN2002451, SPLEN2002462, SPLEN2002463, SPLEN2002467, SPLEN2002477, SPLEN2002493, SPLEN2002662, SPLEN2002695, SPLEN2002707, SPLEN2002744,

SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004220, SPLEN2004343, SPLEN2004346, SPLEN2004368, SPLEN2004611, SPLEN2004662, SPLEN2004773, SPLEN2004844, SPLEN2004880, SPLEN2004984, SPLEN2005009, SPLEN2005227, SPLEN2005272, SPLEN2005342, SPLEN2005416, SPLEN2005429, SPLEN2005450, SPLEN2005474, SPLEN2005560, SPLEN2005727, SPLEN2005783, SPLEN2005790, SPLEN2005818, SPLEN2005838, SPLEN2005869, SPLEN2005927, SPLEN2005939, SPLEN2006143, SPLEN2006232, SPLEN2006268, SPLEN2006283, SPLEN2006305, SPLEN2006325,

SPLEN2006374, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007498, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008496, SPLEN2008591, SPLEN2008786, SPLEN2009081, SPLEN2009088, SPLEN2009163, SPLEN2009315, SPLEN2009340, SPLEN2009512, SPLEN2009513, SPLEN2009541, SPLEN2009548, SPLEN2009581, SPLEN2009733, SPLEN2009803, SPLEN2009884, SPLEN2009918, SPLEN2009970, SPLEN2010004, SPLEN2010015, SPLEN2010112,

SPLEN2010119, SPLEN2010146, SPLEN2010187, SPLEN2010195, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011678, SPLEN2011737, SPLEN2011758, SPLEN2011766, SPLEN2011805, SPLEN2011820, SPLEN2011830, SPLEN2011931, SPLEN2011981, SPLEN2012175, SPLEN2012179, SPLEN2012185, SPLEN2012234, SPLEN2012453, SPLEN2012523, SPLEN2012524, SPLEN2012525, SPLEN2012571, SPLEN2012606,

SPLEN2012611, SPLEN2012619, SPLEN2012725, SPLEN2012743, SPLEN2012756, SPLEN2012800, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014080, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014911, SPLEN2014919, SPLEN2014920,

SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016304, SPLEN2016356, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016773, SPLEN2016781, SPLEN2016840,

SPLEN2016863, SPLEN2016922, SPLEN2016932, SPLEN2016972, SPLEN2016973, SPLEN2017043, SPLEN2017.104, SPLEN2017121, SPLEN2017147, SPLEN2017187, SPLEN2017189, SPLEN2017212, SPLEN2017318, SPLEN2017351, SPLEN2017426, SPLEN2017452, SPLEN2017538, SPLEN2017592, SPLEN2017613, SPLEN2017643, SPLEN2017687, SPLEN2017740, SPLEN2017781, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019169, SPLEN2019257, SPLEN2019311,

SPLEN2019323, SPLEN2019349, SPLEN2019375, SPLEN2019379, SPLEN2019405, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021194, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983,

SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138,

SPLEN2028365, SPLEN2028417, SPLEN2028424, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031547, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081,

SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039379, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645,

SPLEN2041700, SPLEN2041720, SPLEN2041977, SPLEN2042051, SPLEN2042303, SPLEN2042521, SPLEN2042535, SPLEN2042598, SPLEN2042714, SPLEN2042920, STOMA1000013, STOMA1000047, STOMA1000052, STOMA1000074, STOMA1000091, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000121, STOMA2000148, STOMA2000168, STOMA2000183, STOMA2000188, STOMA2000257, STOMA2000279, STOMA2000289, STOMA2000383, STOMA2000386, STOMA2000395, STOMA2000396, STOMA2000478, STOMA2000482, STOMA2000529, STOMA2000539, STOMA2000567, STOMA2000640, STOMA2000678, STOMA2000686, STOMA2000888, STOMA2001025, STOMA2001389, STOMA2002052,

STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049,

SYNOV1000118, SYNOV1000124, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001144, SYNOV2001212, SYNOV2001239, SYNOV2001251, SYNOV2001262, SYNOV2001300, SYNOV2001356, SYNOV2001374, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005673, SYNOV2005817, SYNOV2005962, SYNOV2006430,

SYNOV2006620, SYNOV2007758, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989,

SYNOV4006256, SYNOV4006327, SYNOV4006542, SYNOV4007012, SYNOV4007215, SYNOV4007360, SYNOV4007430, SYNOV4007521, SYNOV4007553, SYNOV4007711, SYNOV4008245, SYNOV4008336, SYNOV4009129, SYNOV4009139, SYNOV4009298, SYNOV4009575, T1ESE2000116, T1ESE2000609, T1ESE2000904, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428,

TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002273, TESOP2002451, TESOP2002489,

TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000023,

TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000163, TESTI1000168, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000172, TESTI2000177, TESTI2000179,

TESTI2000184, TESTI2000207, TESTI2000208, TESTI2000221, TESTI2000238, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049,

TESTI2001082, TESTI2001120, TESTI2001134, TESTI2001141, TESTI2001153, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001827, TESTI2001829, TESTI2001852, TESTI2001862, TESTI2001869, TESTI2001879, TESTI2001894, TESTI2001898, TESTI2001915, TESTI2001950,

TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002707, TESTI2002724, TESTI2002729, TESTI2002752, TESTI2002753, TESTI2002789,

TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372, TESTI2003376,

TESTI2003413, TESTI2003418, TESTI2003419, TESTI2003432, TESTI2003454, TESTI2003475, TESTI2003476, TESTI2003498, TESTI2003512, TESTI2003533, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004031, TESTI2004073, TESTI2004080, TESTI2004085, TESTI2004089, TESTI2004111, TESTI2004119, TESTI2004122, TESTI2004153, TESTI2004163,

TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004737, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994, TESTI2004999,

TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005363, TESTI2005376, TESTI2005378, TESTI2005380, TESTI2005391, TESTI2005395, TESTI2005396, TESTI2005408, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005767, TESTI2005775, TESTI2005784, TESTI2005788, TESTI2005827, TESTI2005835,

TESTI2005860, TESTI2005892, TESTI2005908, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006051, TESTI2006067, TESTI2006083, TESTI2006097, TESTI2006109, TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006453, TESTI2006457, TESTI2006460, TESTI2006465, TESTI2006483,

TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006748, TESTI2006774, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007311, TESTI2007320, TESTI2007346, TESTI2007358, TESTI2007402, TESTI2007405, TESTI2007407,

TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007951, TESTI2007972, TESTI2007973, TESTI2007998, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394,

TESTI2008405, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009474, TESTI2009477, TESTI2009497, TESTI2009502, TESTI2009511, TESTI2009520, TESTI2009544, TESTI2009545, TESTI2009551,

TESTI2009585, TESTI2009588, TESTI2009683, TESTI2009727, TESTI2009739, TESTI2009785, TESTI2009812, TESTI2009835, TESTI2009853, TESTI2009935, TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605,

TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012155, TESTI2012171, TESTI2012207, TESTI2012231, TESTI2012244, TESTI2012289, TESTI2012308, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231,

TESTI2013257, TESTI2013264, TESTI2013268, TESTI2013295, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979,

TESTI2014988, TESTI2015042, TESTI2015092, TESTI2015105, TESTI2015180, TESTI2015213, TESTI2015246, TESTI2015249, TESTI2015327, TESTI2015331, TESTI2015335, TESTI2015350, TESTI2015375, TESTI2015437, TESTI2015590, TESTI2015610, TESTI2015626, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863,

TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018462, TESTI2018475, TESTI2018482, TESTI2018521, TESTI2018565,

TESTI2018581, TESTI2018611, TESTI2018687, TESTI2018697, TESTI2018838, TESTI2018867, TESTI2018941, TESTI2018957, TESTI2018974, TESTI2018975, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379,

TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021122, TESTI2021124, TESTI2021138, TESTI2021297, TESTI2021315, TESTI2021358, TESTI2021425, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203,

TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473,

TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025454, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104,

TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562,

TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342,

TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401,

TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034940, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951,

TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276,

TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750,

TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439,

TESTI2046456, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279,

TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723, TESTI3000002, TESTI3000005, TESTI4000014, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394,

TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002072, TESTI4002141, TESTI4002195, TESTI4002202, TESTI4002290, TESTI4002319, TESTI4002491, TESTI4002520, TESTI4002552, TESTI4002647, TESTI4002703, TESTI4002754, TESTI4002868, TESTI4002878, TESTI4002889,

TESTI4002988, TESTI4003150, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326,

TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840,

TESTI4008935, TESTI4008993, TESTI4009022, TESTI4009028, TESTI4009034, TESTI4009123, TESTI4009160, TESTI4009215, TESTI4009283, TESTI4009286, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010851, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745,

TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013474, TESTI4013602, TESTI4013667, TESTI4013675, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014801, TESTI4014818,

TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152,

TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942,

TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612,

TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633,

TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363,

TESTI4040535, TESTI4040559, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043140, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682,

TESTI4044704, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000017, THYMU1000025, THYMU1000029, THYMU1000033, THYMU1000041, THYMU1000060, THYMU1000074, THYMU1000083, THYMU1000098, THYMU1000103, THYMU1000105, THYMU1000109, THYMU1000136, THYMU1000142,

THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000329, THYMU1000331, THYMU1000336, THYMU1000366, THYMU1000374, THYMU1000382, THYMU1000393, THYMU1000394, THYMU1000399, THYMU1000426, THYMU1000440, THYMU1000459, THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231,

THYMU2000236, THYMU2000276, THYMU2000280, THYMU2000300, THYMU2000317, THYMU2000368, THYMU2000369, THYMU2000418, THYMU2000436, THYMU2000440, THYMU2000489, THYMU2000492, THYMU2000519, THYMU2000570, THYMU2000602, THYMU2000660, THYMU2000671, THYMU2000672, THYMU2000684, THYMU2000702, THYMU2000767, THYMU2000775, THYMU2000800, THYMU2000879, THYMU2000932, THYMU2000950, THYMU2000971, THYMU2000982, THYMU2000990, THYMU2001007, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001443, THYMU2001515, THYMU2001521, THYMU2001556, THYMU2001622, THYMU2001727,

THYMU2001819, THYMU2001825, THYMU2001839, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002815, THYMU2002841, THYMU2002852, THYMU2002910, THYMU2002950, THYMU2003012, THYMU2003046, THYMU2003069, THYMU2003107, THYMU2003124, THYMU2003133, THYMU2003166, THYMU2003232, THYMU2003242, THYMU2003249, THYMU2003270, THYMU2003282,

THYMU2003287, THYMU2003336, THYMU2003366, THYMU2003397, THYMU2003419, THYMU2003440, THYMU2003446, THYMU2003470, THYMU2003479, THYMU2003499, THYMU2003542, THYMU2003632, THYMU2003650, THYMU2003700, THYMU2003760, THYMU2003803, THYMU2003855, THYMU2003907, THYMU2003932, THYMU2003981, THYMU2004003, THYMU2004008, THYMU2004042, THYMU2004108, THYMU2004111, THYMU2004123, THYMU2004139, THYMU2004169, THYMU2004284, THYMU2004320, THYMU2004336, THYMU2004356, THYMU2004410, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004916, THYMU2004986, THYMU2005001, THYMU2005003,

THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006813, THYMU2006913, THYMU2006946,

THYMU2006965, THYMU2007025, THYMU2007036, THYMU2007060, THYMU2007065, THYMU2007095, THYMU2007112, THYMU2007146, THYMU2007179, THYMU2007258, THYMU2007307, THYMU2007308, THYMU2007339, THYMU2007350, THYMU2007384, THYMU2007415, THYMU2007467, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007952, THYMU2007969, THYMU2008035, THYMU2008036, THYMU2008049, THYMU2008072, THYMU2008111, THYMU2008145, THYMU2008149, THYMU2008191, THYMU2008207, THYMU2008226, THYMU2008282, THYMU2008321, THYMU2008339, THYMU2008350, THYMU2008351,

THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010448, THYMU2010519, THYMU2010637, THYMU2010671, THYMU2010699, THYMU2010705, THYMU2010779,

THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012024, THYMU2012073, THYMU2012104, THYMU2012113, THYMU2012273, THYMU2012401, THYMU2012492, THYMU2012507, THYMU2012565, THYMU2012574, THYMU2012631, THYMU2012665, THYMU2012690, THYMU2012701, THYMU2012807,

THYMU2012826, THYMU2012882, THYMU2012891, THYMU2012902, THYMU2012919, THYMU2013023, THYMU2013047, THYMU2013051, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015762, THYMU2015775, THYMU2015825, THYMU2015918, THYMU2015943,

THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054,

THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572,

THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249,

THYMU2031258, THYMU2031341, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554,

THYMU2035710, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824,

THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632,

THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717,

THYMU3011827, THYMU3012402, THYMU3012907, THYMU3012983, THYMU3013114, THYMU3013197, THYMU3013241, THYMU3013386, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015759, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021586, THYMU3021755, THYMU3021900,

THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612,

THYMU3031868, THYMU3031878, THYMU3032032, THYMU3032798, THYMU3032867, THYMU3033402, THYMU3033626, THYMU3033630, THYMU3033649, THYMU3033754, THYMU3033759, THYMU3034078, THYMU3034099, THYMU3034453, THYMU3034616, THYMU3034671, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168,

THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000001, TKIDN1000044, TKIDN1000062,

TKIDN1000066, TKIDN1000164, TKIDN1000171, TKIDN1000192, TKIDN2000127, TKIDN2000240, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2002738, TKIDN2003044, TKIDN2003059, TKIDN2003062, TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005487, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200,

TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015423, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002046, TLIVE2002336, TLIVE2002338, TLIVE2002562, TLIVE2002690, TLIVE2002882, TLIVE2003197, TLIVE2003200, TLIVE2003225, TLIVE2003381,

TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018,

TRACH1000030, TRACH1000038, TRACH1000057, TRACH1000063, TRACH1000074, TRACH1000100, TRACH1000106, TRACH1000125, TRACH1000140, TRACH1000144, TRACH1000181, TRACH1000193, TRACH1000205, TRACH1000212, TRACH1000240, TRACH2000002, TRACH2000015, TRACH2000024, TRACH2000032, TRACH2000045, TRACH2000047, TRACH2000079, TRACH2000081, TRACH2000083, TRACH2000148, TRACH2000185, TRACH2000196, TRACH2000235, TRACH2000237, TRACH2000243, TRACH2000248, TRACH2000250, TRACH2000259, TRACH2000272, TRACH2000282, TRACH2000287, TRACH2000289, TRACH2000306, TRACH2000312, TRACH2000321, TRACH2000336, TRACH2000359, TRACH2000393, TRACH2000411, TRACH2000420, TRACH2000461, TRACH2000496, TRACH2000497, TRACH2000502, TRACH2000522,

TRACH2000660, TRACH2000665, TRACH2000675, TRACH2000677, TRACH2000702, TRACH2000703, TRACH2000767, TRACH2000775, TRACH2000780, TRACH2000807, TRACH2000862, TRACH2000873, TRACH2000894, TRACH2000898, TRACH2000920, TRACH2000923, TRACH2000926, TRACH2000944, TRACH2000959, TRACH2000972, TRACH2000981, TRACH2000986, TRACH2001037, TRACH2001060, TRACH2001085, TRACH2001101, TRACH2001109, TRACH2001121, TRACH2001147, TRACH2001154, TRACH2001188, TRACH2001192, TRACH2001249, TRACH2001275, TRACH2001314, TRACH2001335, TRACH2001339, TRACH2001341, TRACH2001347, TRACH2001395, TRACH2001400, TRACH2001403, TRACH2001416, TRACH2001428, TRACH2001432, TRACH2001443, TRACH2001463, TRACH2001474, TRACH2001523, TRACH2001525,

TRACH2001532, TRACH2001549, TRACH2001592, TRACH2001596, TRACH2001612, TRACH2001621, TRACH2001646, TRACH2001651, TRACH2001684, TRACH2001933, TRACH2001968, TRACH2001996, TRACH2002043, TRACH2002054, TRACH2002100, TRACH2002138, TRACH2002333, TRACH2002537, TRACH2002664, TRACH2002784, TRACH2002803, TRACH2002840, TRACH2002847, TRACH2002853, TRACH2002862, TRACH2002954, TRACH2002960, TRACH2002988, TRACH2003070, TRACH2003272, TRACH2003323, TRACH2003347, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458,

TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005811, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007399, TRACH2007483, TRACH2007542, TRACH2007577, TRACH2007674, TRACH2007676, TRACH2007688, TRACH2007733, TRACH2007754, TRACH2007816, TRACH2007894, TRACH2007969, TRACH2007988, TRACH2008081,

TRACH2008112, TRACH2008113, TRACH2008127, TRACH2008130, TRACH2008278, TRACH2008300, TRACH2008472, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965,

TRACH2010974, TRACH2011057, TRACH2011093, TRACH2011113, TRACH2011290, TRACH2011293, TRACH2011302, TRACH2011500, TRACH2011574, TRACH2011894, TRACH2011956, TRACH2012000, TRACH2012138, TRACH2012242, TRACH2012370, TRACH2012376, TRACH2012387, TRACH2012497, TRACH2012536, TRACH2012562, TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014077, TRACH2014082, TRACH2014124, TRACH2014268, TRACH2014284, TRACH2014337, TRACH2014356, TRACH2014371, TRACH2014442,

TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015823, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034,

TRACH2018084, TRACH2018262, TRACH2018273, TRACH2018278, TRACH2018317, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306,

TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025535, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926,

TRACH3001119, TRACH3001443, TRACH3001550, TRACH3001759, TRACH3002064, TRACH3002107, TRACH3002168, TRACH3002188, TRACH3002192, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003547, TRACH3003586, TRACH3003683, TRACH3003780, TRACH3003805, TRACH3003832, TRACH3003872, TRACH3004068, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840,

TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006800, TRACH3006889, TRACH3006985, TRACH3007097, TRACH3007268, TRACH3007274, TRACH3007277, TRACH3007391, TRACH3007541, TRACH3007595, TRACH3007625, TRACH3007627, TRACH3007689, TRACH3007766, TRACH3007866, TRACH3007995, TRACH3008042, TRACH3008061,

TRACH3008093, TRACH3008201, TRACH3008508, TRACH3008535, TRACH3008629, TRACH3008632, TRACH3008688, TRACH3008697, TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942,

TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018,

TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423,

TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400,

TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063,

TRACH3037067, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143,

UMVEN1000156, UMVEN2000046, UMVEN2000069, UMVEN2000121, UMVEN2000152, UMVEN2000354, UMVEN2000453, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000182, UTERU1000183, UTERU1000187, UTERU1000192, UTERU1000249, UTERU1000337, UTERU1000339, UTERU1000384, UTERU2000023, UTERU2000047, UTERU2000074, UTERU2000099, UTERU2000154, UTERU2000197, UTERU2000218, UTERU2000238, UTERU2000243, UTERU2000260, UTERU2000300, UTERU2000329, UTERU2000332, UTERU2000338, UTERU2000349, UTERU2000377, UTERU2000393, UTERU2000418, UTERU2000424,

UTERU2000465, UTERU2000485, UTERU2000517, UTERU2000524, UTERU2000537, UTERU2000539, UTERU2000541, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002198, UTERU2002332, UTERU2002410, UTERU2002473, UTERU2002547, UTERU2002693, UTERU2002733, UTERU2002736, UTERU2002737, UTERU2002826, UTERU2002841, UTERU2002964, UTERU2002993, UTERU2003035, UTERU2003057,

UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004037, UTERU2004039, UTERU2004061, UTERU2004073, UTERU2004163, UTERU2004197, UTERU2004299, UTERU2004461, UTERU2004520, UTERU2004564, UTERU2004664, UTERU2004688, UTERU2004698, UTERU2004807, UTERU2004861, UTERU2004929, UTERU2005004, UTERU2005050, UTERU2005069, UTERU2005074, UTERU2005179, UTERU2005292, UTERU2005346, UTERU2005354, UTERU2005446, UTERU2005449, UTERU2005450, UTERU2005533, UTERU2005601, UTERU2005664, UTERU2005822, UTERU2005903, UTERU2005905, UTERU2006103, UTERU2006115, UTERU2006137, UTERU2006182, UTERU2006400, UTERU2006412,

UTERU2006429, UTERU2006486, UTERU2006524, UTERU2006547, UTERU2006568, UTERU2006593, UTERU2006643, UTERU2006651, UTERU2006705, UTERU2006899, UTERU2007004, UTERU2007075, UTERU2007081, UTERU2007128, UTERU2007267, UTERU2007444, UTERU2007499, UTERU2007639, UTERU2007724, UTERU2007924, UTERU2007942, UTERU2008018, UTERU2008019, UTERU2008027, UTERU2008040, UTERU2008077, UTERU2008130, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008938, UTERU2008939, UTERU2008962, UTERU2009094, UTERU2009120, UTERU2009131, UTERU2009147, UTERU2009206, UTERU2009283, UTERU2009335, UTERU2009414,

UTERU2009435, UTERU2009483, UTERU2009510, UTERU2009538, UTERU2009540, UTERU2009776, UTERU2009904, UTERU2009951, UTERU2009972, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012230, UTERU2012252, UTERU2012286, UTERU2012333, UTERU2012407, UTERU2012526, UTERU2012581, UTERU2012610, UTERU2012615, UTERU2012688, UTERU2012703,

UTERU2012715, UTERU2012741, UTERU2012767, UTERU2012786, UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001, UTERU2014024, UTERU2014167, UTERU2014223, UTERU2014398, UTERU2014464, UTERU2014548, UTERU2014601, UTERU2014631, UTERU2014668, UTERU2014678, UTERU2014728, UTERU2014898, UTERU2014998, UTERU2015062, UTERU2015087, UTERU2015190, UTERU2015198, UTERU2015202, UTERU2015405, UTERU2015640, UTERU2015653, UTERU2015830, UTERU2015880, UTERU2016147, UTERU2016157, UTERU2016426, UTERU2016464, UTERU2016669, UTERU2016757,

UTERU2016761, UTERU2016799, UTERU2016822, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018609, UTERU2018674, UTERU2018712, UTERU2018784, UTERU2018789, UTERU2018811, UTERU2018867, UTERU2018881, UTERU2018884, UTERU2018955, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964,

UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023175, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941,

UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530,

UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135,

UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722,

UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176,

UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the NCRRM, NCRRP, NESOP, NETRP, NHNPC, and NOVAR libraries were analyzed for expression frequency. The analysis results are described below. Each is a relative value, where the expression level over all tissues is taken as 100. The larger this value, the greater the level of expression.

TABLE 34

| Clone Name | NCRRM | NCRRP | NESOP | NETRP | NHNPC | NOVAR |
|---|---|---|---|---|---|---|
| 3NB692000281 | 0 | 0 | 4.684 | 0 | 5.492 | 0 |
| 3NB692000374 | 0 | 0 | 0 | 0 | 27.059 | 0 |
| 3NB692000912 | 0 | 0 | 0 | 0 | 11.723 | 0 |
| 3NB692001334 | 0 | 0 | 0 | 5.204 | 6.697 | 0 |
| 3NB692001339 | 0 | 0 | 49.901 | 0 | 0 | 0 |
| ADRGL2000006 | 0 | 0 | 0 | 0 | 0 | 24.778 |
| ADRGL2009273 | 0 | 0 | 29.556 | 0 | 0 | 0 |
| ASTRO1000096 | 0 | 0 | 0 | 65.097 | 0 | 0 |
| ASTRO2000014 | 0 | 35.063 | 0 | 0 | 0 | 0 |
| ASTRO2000801 | 86.606 | 0 | 0 | 0 | 0 | 0 |
| ASTRO2002202 | 0 | 0 | 0 | 0 | 0 | 54.926 |
| ASTRO2007047 | 0 | 0 | 0 | 0 | 52.143 | 0 |
| ASTRO2008216 | 0 | 0 | 0 | 20.620 | 0 | 0 |
| ASTRO2008508 | 0 | 0 | 0 | 58.115 | 0 | 0 |
| ASTRO2016491 | 0 | 0 | 0 | 3.254 | 0 | 0 |
| BGGI12000054 | 0 | 0 | 5.011 | 1.522 | 0 | 0 |
| BGGI12000616 | 0 | 0 | 0 | 0 | 0 | 21.310 |
| BLADE2002744 | 0 | 0 | 0 | 15.772 | 0 | 0 |
| BNGH42000360 | 0 | 0 | 0 | 0 | 0 | 3.749 |
| BNGH42007709 | 0 | 0 | 0 | 1.487 | 5.743 | 0 |
| BRACE1000131 | 0 | 0 | 0 | 0 | 25.988 | 0 |
| BRACE2001692 | 0 | 0 | 12.883 | 0 | 0 | 0 |
| BRACE2002677 | 0 | 0 | 0 | 12.475 | 0 | 0 |
| BRACE2002685 | 0 | 0 | 72.930 | 0 | 0 | 0 |
| BRACE2003172 | 0 | 0 | 0 | 0 | 0 | 7.746 |
| BRACE2003827 | 0 | 0 | 0 | 0 | 0 | 20.882 |
| BRACE2005433 | 0 | 0 | 0 | 74.625 | 0 | 0 |
| BRACE2005875 | 0 | 0 | 91.597 | 0 | 0 | 0 |
| BRACE2006344 | 0 | 0 | 76.055 | 0 | 0 | 0 |
| BRACE2006397 | 0 | 0 | 0 | 0 | 7.365 | 0 |
| BRACE2006900 | 0 | 0 | 0 | 3.325 | 0 | 0 |
| BRACE2007109 | 0 | 0 | 20.309 | 0 | 0 | 0 |
| BRACE2008097 | 0 | 0 | 0 | 31.024 | 0 | 0 |
| BRACE2008385 | 0 | 0 | 0 | 53.984 | 0 | 0 |
| BRACE2009517 | 0 | 0 | 0 | 17.526 | 0 | 0 |
| BRACE2016335 | 0 | 0 | 0 | 90.103 | 0 | 0 |
| BRACE2044949 | 0 | 0 | 0 | 0 | 97.234 | 0 |
| BRACE3019941 | 0 | 0 | 0 | 0 | 72.896 | 0 |
| BRALZ2009482 | 0 | 31.729 | 0 | 0 | 0 | 8.857 |
| BRAMY2006397 | 0 | 0 | 0 | 18.146 | 0 | 0 |
| BRAMY2020427 | 0 | 0 | 0 | 0 | 0 | 14.113 |
| BRAMY2023719 | 0 | 0 | 0 | 38.634 | 0 | 0 |
| BRAMY2027739 | 0 | 0 | 0 | 0 | 33.576 | 0 |
| BRAMY2036387 | 0 | 0 | 0 | 86.510 | 0 | 0 |
| BRAMY2040095 | 0 | 0 | 0 | 15.462 | 0 | 8.147 |
| BRAMY2047420 | 0 | 0 | 21.553 | 0 | 0 | 0 |
| BRAWH1000040 | 0 | 0 | 0 | 0 | 17.168 | 0 |
| BRAWH1000093 | 0 | 0 | 0 | 11.921 | 0 | 0 |
| BRAWH2000588 | 0 | 0 | 0 | 8.107 | 0 | 0 |
| BRAWH2001092 | 0 | 0 | 0 | 0 | 4.385 | 0 |
| BRAWH2007800 | 0 | 0 | 0 | 30.357 | 0 | 0 |
| BRAWH2008577 | 0 | 0 | 0 | 22.974 | 0 | 0 |
| BRAWH2009490 | 0 | 0 | 0 | 0 | 84.417 | 0 |
| BRAWH2012161 | 0 | 0 | 81.923 | 0 | 0 | 0 |
| BRAWH2016269 | 0 | 0 | 0 | 0 | 32.893 | 0 |
| BRAWH2017685 | 0 | 0 | 0 | 0 | 25.325 | 0 |
| BRAWH2018526 | 26.126 | 0 | 0 | 0 | 23.035 | 0 |
| BRAWH3002853 | 0 | 0 | 0 | 4.780 | 0 | 0 |
| BRCAN2009432 | 0 | 0 | 0 | 8.509 | 0 | 0 |
| BRHIP2000534 | 0 | 0 | 0 | 8.847 | 0 | 8.158 |
| BRHIP2010593 | 0 | 0 | 0 | 0 | 0 | 83.465 |
| BRHIP2011491 | 0 | 0 | 0 | 0 | 0 | 19.701 |
| BRHIP2019186 | 0 | 0 | 0 | 0 | 0 | 47.038 |
| BRHIP3007223 | 0 | 0 | 42.899 | 0 | 0 | 0 |
| BRHIP3009753 | 98.813 | 0 | 0 | 0 | 0 | 0 |
| BRHIP3018784 | 0 | 0 | 0 | 67.175 | 0 | 0 |
| BRSSN2015238 | 0 | 0 | 0 | 22.121 | 0 | 0 |
| BRSTN1000083 | 0 | 54.516 | 0 | 0 | 0 | 0 |
| BRSTN2012069 | 0 | 0 | 1.409 | 0 | 0.472 | 5.861 |
| BRSTN2012174 | 0 | 0 | 0 | 0 | 5.046 | 14.462 |
| CTONG1000010 | 0 | 0 | 0 | 41.893 | 0 | 0 |
| CTONG1000052 | 0 | 0 | 0 | 3.904 | 0 | 0 |
| CTONG1000094 | 33.063 | 0 | 0 | 0 | 0 | 0 |
| CTONG1000218 | 0 | 0 | 0 | 3.111 | 0 | 0 |
| CTONG2002744 | 0 | 0 | 12.530 | 0 | 0 | 0 |
| CTONG2007500 | 0 | 0 | 0 | 4.860 | 0 | 0 |
| CTONG2012452 | 0 | 0 | 0 | 0 | 8.885 | 0 |
| CTONG2015091 | 0 | 0 | 0 | 0 | 20.262 | 0 |
| CTONG2016185 | 0 | 0 | 0 | 16.799 | 0 | 0 |
| CTONG2017604 | 0 | 0 | 0 | 77.625 | 0 | 0 |
| CTONG2019788 | 0 | 0 | 0 | 0 | 0 | 72.878 |
| CTONG2028124 | 0 | 0 | 0 | 9.743 | 0 | 0 |
| CTONG2028208 | 0 | 0 | 0 | 22.882 | 0 | 0 |
| CTONG3002412 | 0 | 0 | 0 | 69.150 | 0 | 0 |
| CTONG3008894 | 0 | 0 | 0 | 4.697 | 0 | 0 |
| CTONG3009028 | 0 | 0 | 0 | 20.798 | 0 | 0 |
| D30ST3000169 | 0 | 0 | 0 | 3.162 | 0 | 0 |
| D90ST2003762 | 0 | 0 | 0 | 14.084 | 0 | 0 |
| DFNES2000268 | 0 | 0 | 0 | 14.271 | 0 | 0 |
| DFNES2000443 | 57.628 | 0 | 0 | 0 | 0 | 0 |
| FCBBF1000294 | 0 | 0 | 0 | 6.957 | 0 | 0 |
| FCBBF2000087 | 0 | 0 | 61.380 | 0 | 0 | 0 |
| FCBBF3001470 | 0 | 0 | 28.883 | 0 | 0 | 0 |
| FCBBF3003800 | 0 | 0 | 0 | 0 | 9.828 | 0 |
| FCBBF3004041 | 0 | 0 | 0 | 0 | 93.106 | 0 |
| FCBBF3009541 | 0 | 35.198 | 0 | 0 | 0 | 0 |
| FEBRA2000733 | 0 | 0 | 0 | 1.089 | 0 | 0 |
| FEBRA2001715 | 0 | 0 | 0 | 0 | 24.879 | 0 |
| FEBRA2001745 | 0 | 0 | 0 | 0 | 31.268 | 29.870 |
| FEBRA2002933 | 0 | 31.597 | 0 | 0 | 0 | 0 |
| FEBRA2003100 | 55.911 | 0 | 0 | 0 | 0 | 0 |
| FEBRA2003541 | 0 | 0 | 0 | 0 | 38.072 | 0 |
| FEBRA2003726 | 84.540 | 0 | 0 | 0 | 0 | 0 |
| FEBRA2003930 | 0 | 0 | 38.240 | 0 | 0 | 0 |
| FEBRA2005734 | 0 | 0 | 0 | 5.456 | 21.067 | 0 |
| FEBRA2008087 | 0 | 0 | 0 | 0 | 0 | 4.272 |
| FEBRA2008583 | 0 | 0 | 0 | 0 | 6.596 | 0 |
| FEBRA2009437 | 0 | 0 | 0 | 0 | 90.916 | 0 |
| FEBRA2013570 | 0 | 0 | 0 | 72.161 | 0 | 0 |
| FEBRA2018476 | 0 | 0 | 0 | 7.060 | 0 | 0 |
| FEBRA2019711 | 0 | 0 | 0 | 13.664 | 0 | 0 |
| FELIV1000153 | 0 | 0 | 3.509 | 0 | 0 | 3.931 |
| HCHON2000056 | 0 | 0 | 0 | 5.498 | 7.077 | 0 |
| HCHON2000087 | 0 | 0 | 0 | 3.998 | 0 | 0 |
| HCHON2000295 | 0 | 0 | 27.959 | 0 | 0 | 0 |
| HCHON2000815 | 0 | 0 | 0 | 18.046 | 0 | 0 |
| HCHON2001359 | 0 | 0 | 0 | 5.501 | 0 | 0 |
| HCHON2004002 | 0 | 0 | 0 | 5.073 | 0 | 0 |
| HEART1000173 | 0 | 0 | 0 | 0 | 65.485 | 0 |
| HHDPC1000114 | 0 | 0 | 15.460 | 0 | 0 | 0 |
| HHDPC2000104 | 0 | 0 | 0 | 0 | 0 | 44.094 |
| HHDPC2000456 | 0 | 0 | 0 | 10.033 | 0 | 0 |
| HHDPC2003439 | 0 | 0 | 0 | 1.643 | 0 | 0 |
| HHDPC2003983 | 0 | 0 | 0 | 0 | 0 | 6.637 |
| HLUNG2000501 | 0 | 0 | 18.252 | 0 | 0 | 0 |
| HLUNG2001439 | 74.869 | 0 | 0 | 0 | 0 | 0 |
| HLUNG2002465 | 0 | 0 | 0 | 31.261 | 0 | 0 |
| HLUNG2003778 | 0 | 0 | 0 | 33.333 | 0 | 0 |
| HLUNG2013784 | 0 | 0 | 0 | 0 | 87.184 | 0 |
| HLUNG2017546 | 0 | 0 | 0 | 0 | 0 | 84.114 |
| HSYRA1000119 | 0 | 0 | 0 | 0 | 11.472 | 0 |
| HSYRA2000159 | 0 | 0 | 0 | 5.454 | 0 | 0 |
| HSYRA2001225 | 62.759 | 0 | 0 | 0 | 0 | 0 |
| HSYRA2001476 | 0 | 0 | 0 | 6.239 | 0 | 0 |
| HSYRA2001595 | 0 | 0 | 0 | 0 | 14.213 | 0 |
| HSYRA2001621 | 0 | 0 | 3.904 | 0 | 0 | 0 |
| IMR321000210 | 0 | 0 | 0 | 0 | 0 | 24.159 |
| IMR321000219 | 0 | 0 | 0 | 1.814 | 0 | 0 |
| IMR321000253 | 0 | 0 | 0 | 0 | 25.412 | 0 |
| IMR322000121 | 0 | 0 | 0 | 14.407 | 27.814 | 0 |
| IMR322000223 | 0 | 0 | 0 | 0 | 16.987 | 16.228 |
| IMR322001218 | 0 | 0 | 0 | 0 | 27.932 | 26.683 |
| IMR322001600 | 13.670 | 0 | 0 | 0 | 0 | 0 |
| KIDNE2001467 | 0 | 22.254 | 0 | 0 | 6.503 | 0 |
| KIDNE2003941 | 0 | 0 | 0 | 0 | 12.585 | 0 |
| KIDNE2007328 | 0 | 0 | 0 | 5.717 | 0 | 0 |
| KIDNE2007352 | 0 | 0 | 0 | 2.686 | 0 | 9.908 |
| KIDNE2007944 | 0 | 0 | 0 | 64.955 | 0 | 0 |
| KIDNE2012440 | 0 | 0 | 0 | 0 | 44.165 | 0 |
| KIDNE2014290 | 0 | 0 | 0 | 9.060 | 0 | 0 |
| LIVER1000132 | 0 | 0 | 0 | 0 | 0 | 11.927 |
| MAMGL1000032 | 0 | 0 | 0 | 2.082 | 0 | 3.840 |
| MESAN2000267 | 0 | 0 | 0 | 0 | 10.217 | 0 |
| MESAN2002724 | 0 | 0 | 0 | 5.959 | 0 | 0 |
| MESAN2003622 | 0 | 0 | 0 | 0 | 0 | 14.324 |
| MESAN2006563 | 0 | 0 | 0 | 0 | 34.458 | 0 |
| MESAN2017373 | 0 | 0 | 0 | 0 | 0 | 83.483 |
| NB9N42000342 | 0 | 0 | 0 | 4.659 | 0 | 0 |
| NCRRP1000129 | 0 | 100 | 0 | 0 | 0 | 0 |
| NESOP1000010 | 0 | 0 | 100 | 0 | 0 | 0 |
| NESOP1000087 | 0 | 0 | 85.774 | 0 | 0 | 0 |
| NESOP1000108 | 0 | 0 | 100 | 0 | 0 | 0 |
| NESOP2000231 | 0 | 0 | 100 | 0 | 0 | 0 |
| NESOP2000452 | 0 | 0 | 100 | 0 | 0 | 0 |
| NESOP2000499 | 0 | 0 | 100 | 0 | 0 | 0 |
| NESOP2000504 | 0 | 0 | 96.229 | 0 | 0 | 0 |
| NESOP2000744 | 0 | 0 | 100 | 0 | 0 | 0 |
| NESOP2001144 | 0 | 0 | 100 | 0 | 0 | 0 |
| NESOP2001433 | 0 | 0 | 100 | 0 | 0 | 0 |
| NESOP2001656 | 0 | 0 | 85.181 | 0 | 0 | 0 |
| NESOP2001694 | 0 | 0 | 100 | 0 | 0 | 0 |
| NESOP2001752 | 0 | 0 | 96.772 | 0 | 0 | 0 |
| NESOP2002352 | 0 | 0 | 100 | 0 | 0 | 0 |
| NESOP2002418 | 0 | 0 | 100 | 0 | 0 | 0 |
| NESOP2002487 | 0 | 0 | 100 | 0 | 0 | 0 |
| NESOP2002738 | 0 | 0 | 100 | 0 | 0 | 0 |
| NESOP2002780 | 0 | 0 | 100 | 0 | 0 | 0 |
| NETRP1000034 | 0 | 0 | 0 | 100 | 0 | 0 |
| NETRP1000082 | 0 | 0 | 0 | 100 | 0 | 0 |
| NETRP2000086 | 0 | 0 | 0 | 100 | 0 | 0 |
| NETRP2000182 | 0 | 0 | 0 | 100 | 0 | 0 |
| NETRP2000439 | 0 | 0 | 0 | 100 | 0 | 0 |
| NHNPC1000034 | 0 | 0 | 0 | 0 | 96.118 | 0 |
| NHNPC1000037 | 0 | 0 | 0 | 0 | 100 | 0 |
| NHNPC1000084 | 0 | 0 | 0 | 0 | 100 | 0 |
| NHNPC1000101 | 0 | 0 | 0 | 0 | 100 | 0 |
| NHNPC1000124 | 0 | 0 | 0 | 0 | 100 | 0 |
| NHNPC2000062 | 0 | 0 | 0 | 0 | 52.287 | 0 |
| NHNPC2000187 | 0 | 0 | 0 | 2.924 | 11.291 | 0 |
| NHNPC2000206 | 0 | 0 | 0 | 4.437 | 17.134 | 0 |
| NHNPC2000212 | 0 | 0 | 0 | 0 | 86.954 | 0 |
| NHNPC2000606 | 0 | 0 | 0 | 0 | 100 | 0 |
| NHNPC2000877 | 0 | 0 | 0 | 0 | 100 | 0 |
| NHNPC2001108 | 0 | 0 | 0 | 0 | 97.447 | 0 |
| NHNPC2001223 | 0 | 0 | 0 | 0 | 93.106 | 0 |
| NHNPC2001312 | 0 | 0 | 0 | 0 | 95.257 | 0 |
| NHNPC2001816 | 0 | 0 | 0 | 0 | 100 | 0 |
| NHNPC2001931 | 0 | 0 | 0 | 0 | 11.907 | 0 |
| NHNPC2002565 | 0 | 0 | 0 | 0 | 100 | 0 |
| NHNPC2002749 | 0 | 0 | 0 | 0 | 100 | 0 |
| NOVAR1000015 | 0 | 0 | 0 | 0 | 0 | 72.374 |
| NOVAR1000091 | 0 | 0 | 0 | 0 | 0 | 46.155 |
| NOVAR1000102 | 0 | 0 | 0 | 0 | 0 | 99.094 |
| NOVAR2000038 | 0 | 0 | 0 | 0 | 0 | 14.805 |
| NOVAR2000136 | 0 | 0 | 0 | 0 | 0 | 100 |
| NOVAR2000352 | 0 | 0 | 0 | 0 | 0 | 100 |
| NOVAR2000412 | 0 | 0 | 0 | 0 | 0 | 100 |
| NOVAR2000710 | 0 | 0 | 0 | 0 | 0 | 83.961 |
| NOVAR2000783 | 0 | 0 | 0 | 0 | 0 | 90.145 |
| NOVAR2000962 | 0 | 0 | 0 | 0 | 0 | 100 |
| NOVAR2001108 | 0 | 0 | 0 | 0 | 0 | 100 |
| NOVAR2001620 | 0 | 0 | 0 | 0 | 0 | 100 |
| NOVAR2001783 | 0 | 0 | 0 | 0 | 0 | 95.973 |
| NOVAR2001876 | 0 | 0 | 0 | 0 | 0 | 100 |
| NOVAR2002039 | 0 | 0 | 0 | 0 | 0 | 96.620 |
| NT2NE2000260 | 0 | 0 | 0 | 3.903 | 3.014 | 0 |
| NT2NE2000575 | 0 | 0 | 5.115 | 0 | 0 | 0 |
| NT2NE2000963 | 0 | 0 | 0 | 0 | 0 | 10.935 |
| NT2NE2001000 | 0 | 0 | 0 | 5.561 | 0 | 0 |
| NT2NE2002870 | 0 | 0 | 0 | 0 | 56.954 | 0 |
| NT2RI2000421 | 0 | 0 | 0 | 0 | 75.923 | 0 |
| NT2RI2000727 | 0 | 0 | 0 | 1.672 | 0 | 0 |
| NT2RI2001230 | 65.484 | 0 | 0 | 0 | 0 | 0 |
| NT2RI2001342 | 0 | 0 | 33.584 | 0 | 0 | 0 |
| NT2RI2001540 | 0 | 0 | 1.211 | 0 | 0 | 2.714 |
| NT2RI2002091 | 0 | 0 | 0 | 0 | 0.669 | 0.639 |
| NT2RI2002316 | 0 | 0 | 17.058 | 5.181 | 0 | 0 |
| NT2RI2003304 | 0 | 0 | 0 | 0 | 6.548 | 0 |
| NT2RI2003481 | 0 | 0 | 41.086 | 0 | 0 | 0 |
| NT2RI2003487 | 0 | 0 | 0 | 0 | 93.211 | 0 |
| NT2RI2003556 | 32.299 | 0 | 0 | 0 | 9.492 | 0 |
| NT2RI2003695 | 0 | 0 | 0 | 5.088 | 0 | 0 |
| NT2RI2003738 | 0 | 0 | 0 | 1.130 | 0 | 0 |
| NT2RI2004284 | 0 | 0 | 0 | 0 | 0 | 38.313 |
| NT2RI2005150 | 0 | 0 | 0 | 0 | 34.386 | 0 |
| NT2RI2005368 | 0 | 0 | 0 | 0 | 0 | 48.158 |
| NT2RI2005814 | 0 | 0 | 0 | 0 | 32.496 | 0 |
| NT2RI2006072 | 64.954 | 0 | 0 | 0 | 0 | 0 |
| NT2RI2006127 | 0 | 0 | 0 | 78.050 | 0 | 0 |
| NT2RI2006271 | 0 | 0 | 0 | 0 | 69.688 | 0 |
| NT2RI2006856 | 0 | 0 | 9.966 | 0 | 0 | 0 |
| NT2RI2007084 | 0 | 0 | 0 | 0 | 27.144 | 0 |
| NT2RI2007133 | 33.304 | 33.495 | 0 | 0 | 0 | 0 |
| NT2RI2007148 | 80.174 | 0 | 0 | 0 | 0 | 0 |
| NT2RI2007384 | 0 | 0 | 9.214 | 0 | 0 | 0 |
| NT2RI2007879 | 0 | 0 | 0 | 10.256 | 0 | 0 |
| NT2RI2007884 | 0 | 0 | 0 | 16.405 | 0 | 0 |
| NT2RI2008622 | 0 | 0 | 0 | 20.392 | 0 | 0 |
| NT2RI2008724 | 0 | 0 | 0 | 11.867 | 0 | 0 |
| NT2RI2008749 | 0 | 0 | 0 | 0 | 14.107 | 0 |
| NT2RI2009194 | 0 | 0 | 0 | 0 | 24.520 | 0 |
| NT2RI2009281 | 0 | 0 | 0 | 0 | 35.116 | 0 |
| NT2RI2017529 | 82.371 | 0 | 0 | 0 | 0 | 0 |
| NT2RP6000005 | 0 | 0 | 0 | 0 | 54.879 | 0 |
| NT2RP6000017 | 0 | 0 | 0 | 0 | 0 | 35.661 |
| NT2RP6000078 | 0 | 0 | 0 | 4.647 | 0 | 0 |
| NT2RP7000076 | 0 | 0 | 0 | 0 | 15.206 | 0 |
| NT2RP7000173 | 0 | 0 | 0 | 0 | 13.871 | 0 |
| NT2RP7001856 | 0 | 0 | 0 | 0 | 85.512 | 0 |
| NT2RP7006490 | 0 | 0 | 0 | 0 | 0 | 51.161 |
| NT2RP7006717 | 0 | 0 | 0 | 4.251 | 0 | 0 |
| NT2RP7007177 | 0 | 0 | 0 | 0 | 33.978 | 0 |
| NT2RP7007530 | 0 | 0 | 0 | 0 | 0 | 52.253 |
| NT2RP7008190 | 0 | 0 | 0 | 0 | 33.097 | 0 |
| NT2RP7009147 | 0 | 0 | 33.894 | 0 | 0 | 0 |
| NT2RP7009482 | 39.344 | 0 | 0 | 2.995 | 0 | 0 |
| NT2RP7009498 | 85.550 | 0 | 0 | 0 | 0 | 0 |
| NT2RP7015503 | 0 | 0 | 0 | 0 | 0 | 75.473 |
| NTONG1000246 | 0 | 0 | 0 | 20.208 | 0 | 0 |
| NTONG1000257 | 0 | 0 | 0 | 56.890 | 0 | 0 |
| NTONG2000334 | 0 | 0 | 0 | 0 | 51.012 | 0 |
| NTONG2000800 | 0 | 0 | 56.331 | 0 | 0 | 0 |
| NTONG2001220 | 0 | 0 | 0 | 37.184 | 0 | 0 |
| NTONG2001222 | 0 | 0 | 35.287 | 0 | 0 | 0 |
| NTONG2001362 | 0 | 0 | 74.273 | 0 | 0 | 0 |
| NTONG2001587 | 0 | 0 | 69.673 | 0 | 0 | 0 |
| NTONG2004844 | 0 | 0 | 0 | 0 | 40.435 | 0 |
| OCBBF2000846 | 0 | 0 | 0 | 4.729 | 0 | 0 |
| OCBBF2000982 | 0 | 0 | 0 | 5.635 | 0 | 0 |
| OCBBF2002086 | 0 | 0 | 0 | 20.818 | 0 | 0 |
| OCBBF2003518 | 87.513 | 0 | 0 | 0 | 0 | 0 |
| OCBBF2003925 | 70.735 | 0 | 0 | 0 | 0 | 0 |
| OCBBF2005428 | 0 | 0 | 0 | 0 | 82.251 | 0 |
| OCBBF2006313 | 0 | 0 | 0 | 36.896 | 0 | 0 |
| OCBBF2007354 | 0 | 0 | 0 | 0 | 25.066 | 0 |
| OCBBF2007415 | 0 | 0 | 0 | 0 | 19.979 | 0 |
| OCBBF2008041 | 0 | 0 | 0 | 0 | 52.152 | 0 |
| OCBBF2009242 | 0 | 0 | 0 | 18.297 | 0 | 0 |
| OCBBF2010830 | 0 | 0 | 18.722 | 17.058 | 0 | 0 |
| OCBBF2012821 | 0 | 0 | 53.237 | 0 | 0 | 0 |
| OCBBF2013366 | 0 | 0 | 0 | 0 | 67.349 | 0 |
| OCBBF2015751 | 0 | 0 | 0 | 0 | 70.707 | 0 |
| PANCR1000081 | 0 | 0 | 0 | 1.932 | 0 | 0 |
| PEBLM2000395 | 0 | 0 | 0 | 5.881 | 0 | 0 |
| PEBLM2008861 | 0 | 0 | 0 | 31.108 | 0 | 0 |
| PLACE6000630 | 0 | 0 | 82.098 | 0 | 0 | 0 |
| PLACE6001262 | 0 | 0 | 0 | 56.464 | 0 | 0 |
| PLACE6003038 | 0 | 0 | 0 | 23.320 | 0 | 0 |
| PLACE6004312 | 0 | 0 | 0 | 7.794 | 15.048 | 0 |
| PLACE6005487 | 0 | 0 | 0 | 0 | 93.342 | 0 |
| PLACE6012574 | 0 | 87.267 | 0 | 0 | 0 | 0 |
| PLACE6019932 | 0 | 0 | 92.281 | 0 | 0 | 0 |
| PROST2000079 | 0 | 0 | 0 | 0 | 74.684 | 0 |
| PROST2000138 | 0 | 14.800 | 1.844 | 1.680 | 0 | 0 |
| PROST2002162 | 0 | 0 | 82.370 | 0 | 0 | 0 |
| PROST2004251 | 0 | 76.493 | 0 | 0 | 0 | 0 |
| PROST2016195 | 0 | 0 | 0 | 37.788 | 0 | 0 |
| PUAEN1000322 | 0 | 0 | 27.200 | 0 | 0 | 0 |
| PUAEN2000374 | 0 | 0 | 0 | 2.068 | 0 | 0 |
| SALGL1000024 | 0 | 0 | 0 | 1.964 | 0 | 0 |
| SALGL1000157 | 0 | 0 | 2.767 | 3.361 | 0 | 0 |
| SKMUS1000186 | 0 | 34.269 | 0 | 0 | 0 | 19.133 |
| SKMUS2001528 | 0 | 0 | 0 | 8.111 | 0 | 0 |
| SKNMC2000322 | 0 | 0 | 0 | 0 | 35.235 | 0 |
| SKNMC2000635 | 0 | 0 | 0 | 0 | 4.476 | 0 |
| SMINT1000131 | 0 | 0 | 19.486 | 0 | 0 | 0 |
| SMINT2000545 | 0 | 0 | 0 | 15.318 | 0 | 0 |
| SMINT2000933 | 0 | 0 | 0 | 13.171 | 0 | 0 |
| SMINT2001199 | 0 | 0 | 0 | 64.679 | 0 | 0 |
| SMINT2001818 | 0 | 0 | 0 | 24.229 | 0 | 0 |
| SMINT2002884 | 0 | 0 | 0 | 23.868 | 0 | 0 |
| SMINT2003505 | 0 | 0 | 0 | 20.349 | 0 | 0 |
| SMINT2003905 | 0 | 66.100 | 0 | 0 | 0 | 0 |
| SMINT2004023 | 0 | 0 | 0 | 29.565 | 0 | 0 |
| SMINT2004583 | 0 | 0 | 82.426 | 0 | 0 | 0 |
| SMINT2005688 | 0 | 0 | 0 | 0 | 62.271 | 0 |
| SMINT2008960 | 0 | 0 | 0 | 4.421 | 0 | 0 |
| SMINT2014480 | 0 | 0 | 0 | 10.266 | 0 | 0 |
| SMINT2015306 | 0 | 0 | 0 | 64.679 | 0 | 0 |
| SPLEN1000089 | 0 | 0 | 0 | 25.678 | 0 | 0 |
| SPLEN1000116 | 42.016 | 0 | 0 | 0 | 0 | 0 |
| SPLEN2000505 | 0 | 35.982 | 0 | 0 | 0 | 0 |
| SPLEN2000698 | 0 | 0 | 0 | 0 | 0 | 50.540 |
| SPLEN2000874 | 0 | 0 | 0 | 3.150 | 0 | 0 |
| SPLEN2000882 | 0 | 0 | 0 | 11.351 | 0 | 0 |
| SPLEN2001141 | 0 | 0 | 0 | 24.408 | 0 | 0 |
| SPLEN2001503 | 0 | 0 | 0 | 65.001 | 0 | 0 |
| SPLEN2002467 | 0 | 0 | 0 | 21.051 | 0 | 0 |
| SPLEN2004181 | 0 | 0 | 0 | 50.868 | 0 | 0 |
| SPLEN2004984 | 0 | 0 | 0 | 50.919 | 0 | 0 |
| SPLEN2005342 | 0 | 0 | 0 | 34.473 | 0 | 0 |
| SPLEN2005806 | 0 | 52.211 | 0 | 0 | 0 | 0 |
| SPLEN2006122 | 0 | 0 | 0 | 5.442 | 0 | 0 |
| SPLEN2007276 | 0 | 0 | 0 | 34.275 | 0 | 0 |
| SPLEN2011678 | 96.754 | 0 | 0 | 0 | 0 | 0 |
| SPLEN2012571 | 0 | 0 | 0 | 20.542 | 0 | 0 |
| SPLEN2012800 | 57.993 | 0 | 0 | 0 | 0 | 0 |
| SPLEN2017918 | 0 | 0 | 0 | 0 | 22.748 | 0 |
| SPLEN2021194 | 0 | 0 | 0 | 43.596 | 0 | 0 |
| SPLEN2022522 | 0 | 0 | 0 | 18.320 | 0 | 0 |
| SPLEN2024232 | 0 | 0 | 0 | 42.861 | 0 | 0 |
| SPLEN2028466 | 0 | 0 | 0 | 0 | 10.321 | 0 |
| SPLEN2028914 | 0 | 0 | 0 | 18.044 | 0 | 0 |
| SPLEN2034448 | 0 | 0 | 0 | 33.504 | 0 | 0 |
| STOMA1000091 | 0 | 0 | 0 | 44.949 | 0 | 0 |
| SYNOV1000164 | 0 | 0 | 0 | 49.698 | 0 | 0 |
| SYNOV2001239 | 0 | 0 | 0 | 2.027 | 0 | 7.478 |
| SYNOV2001262 | 0 | 0 | 0 | 13.482 | 0 | 0 |
| SYNOV2001581 | 0 | 0 | 0 | 17.017 | 0 | 0 |
| SYNOV2001648 | 0 | 0 | 0 | 7.002 | 0 | 0 |
| TESTI2000591 | 0 | 0 | 38.854 | 0 | 0 | 43.525 |
| TESTI2001862 | 0 | 0 | 56.260 | 0 | 0 | 0 |
| TESTI2002149 | 0 | 0 | 0 | 0 | 0 | 39.864 |
| TESTI2003005 | 0 | 0 | 0 | 0 | 75.802 | 0 |
| TESTI2003071 | 0 | 0 | 0 | 0 | 0 | 83.156 |
| TESTI2003127 | 0 | 0 | 0 | 68.997 | 0 | 0 |
| TESTI2003756 | 0 | 0 | 0 | 67.792 | 0 | 0 |
| TESTI2004031 | 0 | 34.228 | 8.530 | 0 | 0 | 0 |
| TESTI2006588 | 0 | 0 | 0 | 0 | 0 | 12.903 |
| TESTI2009474 | 0 | 0 | 0 | 13.055 | 0 | 0 |
| TESTI2010513 | 33.353 | 0 | 0 | 0 | 0 | 0 |
| TESTI2012019 | 0 | 0 | 0 | 0 | 0 | 92.398 |
| TESTI2012778 | 47.058 | 0 | 0 | 0 | 0 | 0 |
| TESTI2013231 | 0 | 0 | 0 | 0 | 0 | 47.426 |
| TESTI2015213 | 0 | 0 | 0 | 0 | 12.805 | 0 |
| TESTI2015437 | 0 | 0 | 0 | 0 | 0 | 97.331 |
| TESTI2015626 | 0 | 0 | 0 | 0 | 0 | 7.838 |
| TESTI2017954 | 0 | 0 | 0 | 0 | 16.528 | 0 |
| TESTI2021654 | 0 | 0 | 0 | 63.153 | 0 | 0 |
| TESTI2025013 | 0 | 0 | 0 | 0 | 0 | 84.839 |
| TESTI2026525 | 0 | 0 | 0 | 28.114 | 0 | 0 |
| TESTI2034940 | 0 | 0 | 0 | 1.683 | 6.500 | 0 |
| TESTI2052693 | 0 | 0 | 0 | 0 | 0 | 39.903 |
| TESTI4000014 | 0 | 30.495 | 0 | 0 | 0 | 0 |
| TESTI4009286 | 0 | 0 | 15.424 | 4.684 | 0 | 0 |
| TESTI4013441 | 0 | 13.391 | 0 | 0 | 0 | 0 |
| TESTI4018436 | 0 | 0 | 0 | 33.629 | 0 | 0 |
| THYMU1000016 | 0 | 0 | 8.222 | 2.497 | 0 | 0 |
| THYMU1000359 | 0 | 0 | 0 | 24.247 | 0 | 0 |
| THYMU1000394 | 0 | 0 | 0 | 31.765 | 0 | 0 |
| THYMU2000057 | 0 | 0 | 0 | 43.820 | 0 | 0 |
| THYMU2003012 | 0 | 0 | 0 | 11.326 | 0 | 0 |
| THYMU2003419 | 0 | 0 | 0 | 9.735 | 0 | 0 |
| THYMU2003891 | 0 | 0 | 0 | 12.257 | 0 | 0 |
| THYMU2003981 | 0 | 0 | 0 | 10.124 | 0 | 0 |
| THYMU2006277 | 0 | 99.033 | 0 | 0 | 0 | 0 |
| THYMU2007060 | 0 | 0 | 0 | 55.100 | 0 | 0 |
| THYMU2007886 | 0 | 0 | 0 | 8.948 | 0 | 0 |
| THYMU2009658 | 0 | 0 | 0 | 0 | 0 | 8.349 |
| THYMU2020416 | 0 | 0 | 0 | 88.571 | 0 | 0 |
| THYMU2027125 | 0 | 0 | 0 | 0 | 0 | 4.266 |
| THYMU2032825 | 0 | 0 | 0 | 60.077 | 0 | 0 |
| THYMU3015759 | 0 | 0 | 0 | 7.275 | 0 | 0 |
| THYMU3032798 | 0 | 0 | 0 | 12.495 | 0 | 0 |
| TKIDN2000246 | 0 | 0 | 0 | 0 | 7.590 | 0 |
| TKIDN2002738 | 35.603 | 0 | 0 | 0 | 0 | 0 |
| TLIVE2001327 | 0 | 0 | 0 | 30.308 | 0 | 0 |
| TLIVE2003970 | 0 | 0 | 0 | 0 | 57.706 | 0 |
| TRACH2000079 | 0 | 0 | 0 | 3.500 | 0 | 0 |
| TRACH2000196 | 0 | 0 | 0 | 85.226 | 0 | 0 |
| TRACH2000321 | 0 | 0 | 20.907 | 3.175 | 0 | 0 |
| TRACH2000540 | 0 | 0 | 0 | 36.823 | 0 | 0 |
| TRACH2000780 | 0 | 0 | 0 | 0 | 21.807 | 0 |
| TRACH2001109 | 0 | 0 | 0 | 0 | 5.222 | 0 |
| TRACH2001523 | 0 | 07 | 36.173 | 0 | 0 | 0 |
| TRACH2001810 | 0 | 0 | 0 | 10.036 | 0 | 0 |
| TRACH2002043 | 0 | 90.545 | 0 | 0 | 0 | 0 |
| TRACH2007754 | 0 | 0 | 31.967 | 0 | 0 | 0 |
| TRACH2008081 | 0 | 0 | 0 | 8.255 | 0 | 0 |
| TRACH2011293 | 0 | 0 | 94.999 | 0 | 0 | 0 |
| TRACH3003832 | 0 | 0 | 0 | 78.462 | 0 | 0 |
| TRACH3036942 | 0 | 0 | 0 | 0 | 0 | 5.816 |
| UMVEN2000133 | 0 | 0 | 0 | 11.363 | 0 | 0 |
| UMVEN2000453 | 0 | 0 | 0 | 3.374 | 0 | 0 |
| UTERU1000182 | 68.865 | 0 | 0 | 0 | 0 | 0 |
| UTERU2000095 | 0 | 0 | 0 | 2.674 | 0 | 0 |
| UTERU2000332 | 0 | 0 | 0 | 61.113 | 0 | 0 |
| UTERU2004664 | 0 | 0 | 0 | 0 | 42.338 | 0 |
| UTERU2005593 | 0 | 0 | 0 | 7.253 | 0 | 0 |
| UTERU2008027 | 0 | 0 | 0 | 0 | 95.438 | 0 |
| UTERU2008085 | 0 | 0 | 0 | 0 | 0 | 8.610 |
| UTERU2010417 | 0 | 0 | 0 | 2.037 | 0 | 0 |
| UTERU2011741 | 0 | 0 | 0 | 76.126 | 0 | 0 |
| UTERU2015108 | 0 | 0 | 0 | 40.775 | 0 | 0 |
| UTERU2018884 | 0 | 0 | 0 | 0 | 77.306 | 0 |
| UTERU2033375 | 98.615 | 0 | 0 | 0 | 0 | 0 |

Genes whose expression levels were negligibly low in each of the above-described libraries are shown with their clone names below.

3NB691000018, 3NB691000085, 3NB691000113, 3NB691000116, 3NB691000129, 3NB691000173, 3NB691000191, 3NB692000012, 3NB692000029, 3NB692000102, 3NB692000154, 3NB692000276, 3NB692000305, 3NB692000330, 3NB692000429, 3NB692000484, 3NB692000529, 3NB692000545, 3NB692000973, 3NB692001002, 3NB692001022, 3NB692001034, 3NB692001040, 3NB692001123, 3NB692001267, 3NB692001288, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001520, 3NB692001528, 3NB692001538, 3NB692001548, 3NB692001557, 3NB692001637, 3NB692001719, 3NB692001853, 3NB692001861, 3NB692002051, 3NB692002365, 3NB692002685,

3NB692002806, 3NB692003538, 3NB692004045, 3NB692004724, 3NB692005439, 3NB692006952, 3NB692008178, 3NB692008729, ACTVT2000380, ADIPS1000064, ADIPS2000069, ADIPS2000088, ADIPS2000245, ADIPS2000425, ADRGL1000002, ADRGL1000018, ADRGL1000033, ADRGL1000038, ADRGL1000065, ADRGL1000067, ADRGL1000111, ADRGL1000144, ADRGL1000147, ADRGL1000160, ADRGL1000165, ADRGL1000182, ADRGL2000042, ADRGL2000056, ADRGL2000064, ADRGL2000074, ADRGL2000085, ADRGL2000097, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002477, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767,

ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000165, ASTRO2000011, ASTRO2000046, ASTRO2000095, ASTRO2000141, ASTRO2000191, ASTRO2000278, ASTRO2000372, ASTRO2000381, ASTRO2000403, ASTRO2000417, ASTRO2000480,

ASTRO2000482, ASTRO2000533, ASTRO2000653, ASTRO2000662, ASTRO2000725, ASTRO2000914, ASTRO2001001, ASTRO2001008, ASTRO2001029, ASTRO2001076, ASTRO2001088, ASTRO2001107, ASTRO2001122, ASTRO2001227, ASTRO2001249, ASTRO2001480, ASTRO2001806, ASTRO2001823, ASTRO2002024, ASTRO2002035, ASTRO2002064, ASTRO2002459, ASTRO2002571, ASTRO2002632, ASTRO2002659, ASTRO2002693, ASTRO2002720, ASTRO2002733, ASTRO2002743, ASTRO2002842, ASTRO2003024, ASTRO2003212, ASTRO2003316, ASTRO2003461, ASTRO2003574, ASTRO2003581, ASTRO2003632, ASTRO2003740, ASTRO2003840, ASTRO2003925, ASTRO2003960, ASTRO2004032, ASTRO2004051, ASTRO2004177, ASTRO2004217, ASTRO2004297, ASTRO2004584, ASTRO2004628, ASTRO2004751, ASTRO2004877,

ASTRO2004974, ASTRO2005008, ASTRO2005081, ASTRO2005096, ASTRO2005242, ASTRO2005273, ASTRO2005343, ASTRO2005360, ASTRO2005413, ASTRO2005423, ASTRO2005553, ASTRO2005557, ASTRO2005575, ASTRO2005593, ASTRO2005687, ASTRO2005726, ASTRO2005863, ASTRO2005896, ASTRO2005902, ASTRO2006356, ASTRO2006475, ASTRO2006732, ASTRO2006747, ASTRO2006920, ASTRO2006952, ASTRO2007221, ASTRO2007377, ASTRO2007515, ASTRO2007578, ASTRO2007643, ASTRO2007666, ASTRO2007948, ASTRO2007956, ASTRO2007962, ASTRO2008040, ASTRO2008239, ASTRO2008409, ASTRO2008425, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072,

ASTRO2010582, ASTRO2010615, ASTRO2010799, ASTRO2010819, ASTRO2010962, ASTRO2010996, ASTRO2011149, ASTRO2011422, ASTRO2011426, ASTRO2011437, ASTRO2011461, ASTRO2011834, ASTRO2011893, ASTRO2012552, ASTRO2013124, ASTRO2013585, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014863, ASTRO2014923, ASTRO2014961, ASTRO2015162, ASTRO2015185, ASTRO2015328, ASTRO2015529, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018169, ASTRO2018373, ASTRO2018540, ASTRO2019039, ASTRO2019171, ASTRO3000154, ASTRO3000172,

ASTRO3000177, ASTRO3000301, ASTRO3000474, ASTRO3000482, BEAST2000454, BGGI11000123, BGGI11000193, BGGI11000285, BGGI12000022, BGGI12000067, BGGI12000161, BGGI12000533, BGGI12000544, BGGI12000684, BGGI12000693, BGGI12000839, BGGI12001002, BGGI12001075, BGGI12001097, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002782,

BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006830, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118,

BNGH41000137, BNGH41000153, BNGH41000169, BNGH41000177, BNGH41000190, BNGH41000198, BNGH41000216, BNGH41000235, BNGH42000130, BNGH42000132, BNGH42000405, BNGH42000532, BNGH42000675, BNGH42000739, BNGH42000791, BNGH42000815, BNGH42000994, BNGH42001247, BNGH42001406, BNGH42001576, BNGH42001724, BNGH42002168, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42005968, BNGH42006135, BNGH42006226, BNGH42006234, BNGH42007037, BNGH42007366, BNGH42007460, BNGH42007594, BNGH42007788, BNGH42007798, BNGH42008199, BNGH42008510, BNGH42008603, BNGH42008649,

BNGH42008743, BNGH42008832, BNGH42008850, BNGH42008943, BNGH42009021, BNGH42009025, BRACE1000020, BRACE1000042, BRACE1000047, BRACE1000051, BRACE1000065, BRACE1000070, BRACE1000073, BRACE1000084, BRACE1000087, BRACE1000092, BRACE1000093, BRACE1000101, BRACE1000114, BRACE1000115, BRACE1000124, BRACE1000132, BRACE1000159, BRACE1000166, BRACE1000169, BRACE1000186, BRACE1000187, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000307, BRACE2000331,

BRACE2000332, BRACE2000347, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000753, BRACE2000815, BRACE2000864, BRACE2000885, BRACE2000893, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001117, BRACE2001143, BRACE2001151, BRACE2001154, BRACE2001188, BRACE2001191, BRACE2001197, BRACE2001236, BRACE2001239,

BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001497, BRACE2001508, BRACE2001534, BRACE2001543, BRACE2001564, BRACE2001588, BRACE2001607, BRACE2001626, BRACE2001673, BRACE2001677, BRACE2001705, BRACE2001709, BRACE2001711, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001892, BRACE2001898, BRACE2001923, BRACE2001942, BRACE2001944, BRACE2001954, BRACE2001971, BRACE2001972,

BRACE2001973, BRACE2002026, BRACE2002034, BRACE2002050, BRACE2002081, BRACE2002091, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002392, BRACE2002394, BRACE2002409, BRACE2002431, BRACE2002441, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002661, BRACE2002676, BRACE2002682, BRACE2002695, BRACE2002707, BRACE2002736, BRACE2002752, BRACE2002755, BRACE2002762, BRACE2002772,

BRACE2002792, BRACE2002796, BRACE2002803, BRACE2002807, BRACE2002812, BRACE2002834, BRACE2002849, BRACE2002857, BRACE2002860, BRACE2002861, BRACE2002884, BRACE2002896, BRACE2002948, BRACE2003037, BRACE2003078, BRACE2003097, BRACE2003110, BRACE2003168, BRACE2003199, BRACE2003210, BRACE2003217, BRACE2003254, BRACE2003269, BRACE2003285, BRACE2003304, BRACE2003319, BRACE2003398, BRACE2003420, BRACE2003428, BRACE2003431, BRACE2003512, BRACE2003516, BRACE2003527, BRACE2003539, BRACE2003540, BRACE2003594, BRACE2003598, BRACE2003609, BRACE2003614, BRACE2003628, BRACE2003639, BRACE2003700, BRACE2003713, BRACE2003766, BRACE2003785, BRACE2003788, BRACE2003800, BRACE2003802, BRACE2003825, BRACE2003845,

BRACE2003846, BRACE2003847, BRACE2003848, BRACE2003873, BRACE2003885, BRACE2003887, BRACE2003892, BRACE2003904, BRACE2003905, BRACE2003928, BRACE2003944, BRACE2003954, BRACE2003982, BRACE2003987, BRACE2005034, BRACE2005047, BRACE2005063, BRACE2005083, BRACE2005087, BRACE2005090, BRACE2005099, BRACE2005118, BRACE2005124, BRACE2005138, BRACE2005151, BRACE2005160, BRACE2005169, BRACE2005189, BRACE2005193, BRACE2005196, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005434, BRACE2005448, BRACE2005450, BRACE2005453, BRACE2005457,

BRACE2005460, BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005881, BRACE2005898, BRACE2005904, BRACE2005906, BRACE2005907, BRACE2005911, BRACE2005937, BRACE2005949, BRACE2005981, BRACE2005991, BRACE2006055, BRACE2006072, BRACE2006075, BRACE2006084, BRACE2006089, BRACE2006102, BRACE2006105, BRACE2006122, BRACE2006137, BRACE2006143, BRACE2006162, BRACE2006167, BRACE2006174, BRACE2006240, BRACE2006245, BRACE2006249, BRACE2006258, BRACE2006264, BRACE2006274, BRACE2006281,

BRACE2006298, BRACE2006319, BRACE2006354, BRACE2006363, BRACE2006378, BRACE2006380, BRACE2006393, BRACE2006413, BRACE2006417, BRACE2006453, BRACE2006459, BRACE2006488, BRACE2006514, BRACE2006534, BRACE2006537, BRACE2006540, BRACE2006547, BRACE2006564, BRACE2006587, BRACE2006598, BRACE2006636, BRACE2006685, BRACE2006703, BRACE2006743, BRACE2006753, BRACE2006783, BRACE2006821, BRACE2006833, BRACE2006859, BRACE2006871, BRACE2006909, BRACE2006911, BRACE2006924, BRACE2006935, BRACE2006937, BRACE2006944, BRACE2006951, BRACE2006982, BRACE2006986, BRACE2007013, BRACE2007064, BRACE2007068, BRACE2007087, BRACE2007089, BRACE2007138, BRACE2007174, BRACE2007191, BRACE2007197, BRACE2007201, BRACE2007203,

BRACE2007232, BRACE2007254, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007518, BRACE2007522, BRACE2007527, BRACE2007538, BRACE2007563, BRACE2007567, BRACE2007577, BRACE2007621, BRACE2007640, BRACE2007641, BRACE2007646, BRACE2007663, BRACE2007671, BRACE2007682, BRACE2007685, BRACE2007708, BRACE2007727, BRACE2007749, BRACE2007760, BRACE2007761, BRACE2007764, BRACE2007767, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902,

BRACE2007920, BRACE2007937, BRACE2007944, BRACE2007952, BRACE2008008, BRACE2008015, BRACE2008021, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008358, BRACE2008361, BRACE2008380, BRACE2008399, BRACE2008401, BRACE2008402, BRACE2008410, BRACE2008420, BRACE2008443, BRACE2008473, BRACE2008480, BRACE2008481, BRACE2008488, BRACE2008495, BRACE2008500, BRACE2008553, BRACE2008594, BRACE2008604, BRACE2008615, BRACE2008622, BRACE2008629, BRACE2008653, BRACE2008655, BRACE2008656, BRACE2008706, BRACE2008708, BRACE2008710, BRACE2008754, BRACE2008797, BRACE2008847, BRACE2008857, BRACE2008861,

BRACE2008867, BRACE2008881, BRACE2008883, BRACE2008917, BRACE2008919, BRACE2008941, BRACE2008948, BRACE2008960, BRACE2008968, BRACE2008999, BRACE2009014, BRACE2009016, BRACE2009031, BRACE2009037, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009291, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009533, BRACE2009558, BRACE2009566, BRACE2009591, BRACE2009620, BRACE2009654, BRACE2009721, BRACE2009732,

BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062,

BRACE2012185, BRACE2012222, BRACE2012232, BRACE2012291, BRACE2012317, BRACE2012510, BRACE2012528, BRACE2012625, BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015412, BRACE2015436, BRACE2015741,

BRACE2015756, BRACE2015969, BRACE2015995, BRACE2016058, BRACE2016194, BRACE2016315, BRACE2016325, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017397, BRACE2017410, BRACE2017438, BRACE2017574, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019244, BRACE2019258, BRACE2019327,

BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021014, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022389,

BRACE2022394, BRACE2022435, BRACE2022448, BRACE2022450, BRACE2022549, BRACE2022633, BRACE2022638, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836,

BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385,

BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286,

BRACE2039297, BRACE2039327, BRACE2039362, BRACE2039475, BRACE2039600, BRACE2039607, BRACE2039624, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2045300, BRACE2045428, BRACE2045445,

BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413,

BRACE3003595, BRACE3003698, BRACE3003866, BRACE3003920, BRACE3004046, BRACE3004058, BRACE3004113, BRACE3004150, BRACE3004205, BRACE3004358, BRACE3004371, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005938, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480,

BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010428, BRACE3010435, BRACE3010438, BRACE3010702, BRACE3010862, BRACE3011271, BRACE3011421, BRACE3011447, BRACE3011505,

BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611,

BRACE3019817, BRACE3019828, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256,

BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012,

BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018,

BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486,

BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000354, BRAMY2000388, BRAMY2000411, BRAMY2000508, BRAMY2000562, BRAMY2000585, BRAMY2000653, BRAMY2000814,

BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031,

BRAMY2004058, BRAMY2004183, BRAMY2004335, BRAMY2004351, BRAMY2004352, BRAMY2004363, BRAMY2004387, BRAMY2004492, BRAMY2004521, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458,

BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064,

BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012536, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019,

BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016325, BRAMY2016327, BRAMY2016386, BRAMY2016706, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273,

BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019554, BRAMY2019600, BRAMY2019643, BRAMY2019710, BRAMY2019963, BRAMY2019985, BRAMY2019989, BRAMY2020050, BRAMY2020058, BRAMY2020106, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982,

BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023729, BRAMY2023786, BRAMY2023852, BRAMY2023863, BRAMY2023939, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711,

BRAMY2024715, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027752, BRAMY2027782, BRAMY2028072, BRAMY2028183, BRAMY2028188, BRAMY2028269, BRAMY2028282,

BRAMY2028472, BRAMY2028491, BRAMY2028516, BRAMY2028565, BRAMY2028593, BRAMY2028682, BRAMY2028740, BRAMY2028783, BRAMY2028796, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032087, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506,

BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300,

BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489,

BRAMY2046537, BRAMY2046667, BRAMY2046742, BRAMY2046871, BRAMY2046984, BRAMY2046989, BRAMY2047169, BRAMY2047280, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3002991, BRAMY3003026, BRAMY3003109, BRAMY3003205, BRAMY3003310, BRAMY3003566, BRAMY3003723, BRAMY3004126, BRAMY3004224, BRAMY3004364, BRAMY3004377, BRAMY3004672, BRAMY3004800, BRAMY3004900, BRAMY3004919,

BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010008, BRAMY3010264, BRAMY3010321, BRAMY3010411, BRAMY3010492, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652,

BRAMY4001863, BRAMY4001913, BRAMY4002575, BRAMY4002628, BRASW1000053, BRASW1000125, BRAWH1000001, BRAWH1000002, BRAWH1000007, BRAWH1000037, BRAWH1000045, BRAWH1000083, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417, BRAWH2000443, BRAWH2000460, BRAWH2000471, BRAWH2000476, BRAWH2000482, BRAWH2000488, BRAWH2000494,

BRAWH2000500, BRAWH2000503, BRAWH2000522, BRAWH2000554, BRAWH2000595, BRAWH2000633, BRAWH2000636, BRAWH2000643, BRAWH2000651, BRAWH2000682, BRAWH2000686, BRAWH2000697, BRAWH2000752, BRAWH2000839, BRAWH2000859, BRAWH2000866, BRAWH2000892, BRAWH2000901, BRAWH2000924, BRAWH2000926, BRAWH2000944, BRAWH2000984, BRAWH2001019, BRAWH2001037, BRAWH2001052, BRAWH2001103, BRAWH2001129, BRAWH2001137, BRAWH2001141, BRAWH2001159, BRAWH2001166, BRAWH2001171, BRAWH2001186, BRAWH2001203, BRAWH2001238, BRAWH2001239, BRAWH2001255, BRAWH2001321, BRAWH2001355, BRAWH2001378, BRAWH2001395, BRAWH2001406, BRAWH2001412, BRAWH2001418, BRAWH2001424, BRAWH2001436, BRAWH2001438, BRAWH2001439, BRAWH2001459, BRAWH2001461,

BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003025, BRAWH2003135, BRAWH2003240, BRAWH2003307, BRAWH2003355, BRAWH2003366,

BRAWH2003662, BRAWH2003689, BRAWH2003693, BRAWH2003818, BRAWH2003832, BRAWH2003948, BRAWH2003964, BRAWH2004044, BRAWH2004068, BRAWH2004078, BRAWH2004095, BRAWH2004136, BRAWH2004217, BRAWH2004580, BRAWH2004731, BRAWH2004779, BRAWH2004842, BRAWH2004884, BRAWH2004923, BRAWH2005008, BRAWH2005068, BRAWH2005074, BRAWH2005225, BRAWH2005315, BRAWH2005517, BRAWH2005524, BRAWH2005533, BRAWH2005578, BRAWH2005661, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2006996, BRAWH2007069, BRAWH2007308, BRAWH2007406,

BRAWH2007550, BRAWH2007570, BRAWH2007591, BRAWH2007605, BRAWH2007658, BRAWH2007664, BRAWH2007808, BRAWH2007825, BRAWH2007862, BRAWH2007890, BRAWH2008058, BRAWH2008128, BRAWH2008219, BRAWH2008255, BRAWH2008292, BRAWH2008297, BRAWH2008366, BRAWH2008380, BRAWH2008540, BRAWH2008706, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009304, BRAWH2009307, BRAWH2009360, BRAWH2009393, BRAWH2009485, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010536,

BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014188,

BRAWH2014234, BRAWH2014379, BRAWH2014414, BRAWH2014473, BRAWH2014511, BRAWH2014645, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534,

BRAWH2017542, BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018729, BRAWH2018745, BRAWH2018875, BRAWH2019053, BRAWH2019055, BRAWH2019198, BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002574, BRAWH3002600, BRAWH3002819, BRAWH3003019, BRAWH3003136, BRAWH3003244,

BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461,

BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884,

BRAWH3020928, BRAWH3021012, BRAWH3021545, BRAWH3021574, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556,

BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN1000168,

BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003944, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117,

BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402,

BRCAN2015464, BRCAN2015719, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368,

BRCAN2025712, BRCAN2025982, BRCAN2026117, BRCAN2026197, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028036, BRCAN2028040, BRCAN2028319, BRCAN2028338, BRCAN2028355, BRCAN2028378, BRCAN2028460, BRCAN2028545, BRCAN2028634, BRCAN2028702, BRCOC1000040, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756,

BRCOC2002777, BRCOC2002835, BRCOC2003100, BRCOC2003125, BRCOC2003187, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010123, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229,

BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019910, BRCOC2019934, BRCOC2020122, BRCOC2020142, BRHIP1000058, BRHIP1000072, BRHIP1000104, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506,

BRHIP2000532, BRHIP2000553, BRHIP2000621, BRHIP2000691, BRHIP2000819, BRHIP2000826, BRHIP2000920, BRHIP2001074, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800,

BRHIP2006819, BRHIP2006921, BRHIP2007305, BRHIP2007307, BRHIP2007616, BRHIP2007690, BRHIP2007741, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010487, BRHIP2010571, BRHIP2010610, BRHIP2010723, BRHIP2010744, BRHIP2011080, BRHIP2011120, BRHIP2011199, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750,

BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387, BRHIP2014954, BRHIP2015153, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047,

BRHIP2019149, BRHIP2019177, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020799, BRHIP2020842, BRHIP2020859, BRHIP2020930, BRHIP2021102, BRHIP2021289, BRHIP2021495, BRHIP2021615, BRHIP2021744, BRHIP2021762, BRHIP2021858, BRHIP2021870, BRHIP2021929, BRHIP2022006, BRHIP2022054, BRHIP2022221, BRHIP2022228, BRHIP2022298, BRHIP2022326, BRHIP2022467, BRHIP2022708, BRHIP2022986, BRHIP2023071, BRHIP2023229, BRHIP2023272, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869,

BRHIP2023888, BRHIP2024046, BRHIP2024165, BRHIP2024201, BRHIP2024347, BRHIP2024398, BRHIP2024549, BRHIP2024742, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000017, BRHIP3000060, BRHIP3000070, BRHIP3000081, BRHIP3000111, BRHIP3000131,

BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307,

BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3010135, BRHIP3010289, BRHIP3010529, BRHIP3011082, BRHIP3011241, BRHIP3011269,

BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3019643, BRHIP3019824, BRHIP3019880, BRHIP3019956, BRHIP3020046, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533,

BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000498, BRSSN2000518, BRSSN2000561, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2001869,

BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009518, BRSSN2009627, BRSSN2009630, BRSSN2009702, BRSSN2010019, BRSSN2010110, BRSSN2010587, BRSSN2010596, BRSSN2010830, BRSSN2011262,

BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014610, BRSSN2014636, BRSSN2014685, BRSSN2015199, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581,

BRSSN2018731, BRSSN2018771, BRSSN2018925, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923,

BRSTN2011211, BRSTN2011597, BRSTN2011688, BRSTN2011799, BRSTN2011961, BRSTN2012089, BRSTN2012284, BRSTN2012320, BRSTN2012380, BRSTN2012451, BRSTN2013171, BRSTN2013354, BRSTN2013502, BRSTN2013931, BRSTN2014490, BRSTN2014633, BRSTN2014751, BRSTN2014770, BRSTN2015015, BRSTN2015889, BRSTN2015978, BRSTN2016066, BRSTN2016335, BRSTN2016470, BRSTN2016678, BRSTN2016892, BRSTN2016918, BRSTN2016992, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855,

BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005864, BRTHA2005942, BRTHA2005956, BRTHA2006075, BRTHA2006146, BRTHA2006194,

BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2007998, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466,

BRTHA2012555, BRTHA2012562, BRTHA2012714, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016577, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165,

BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027229, BRTHA2027250, BRTHA2027546, BRTHA2028297, BRTHA2029969, BRTHA2030036, BRTHA2030213, BRTHA2031517, BRTHA2031800, BRTHA2031917,

BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003490, BRTHA3003704, BRTHA3003736,

BRTHA3004307, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011194, BRTHA3011229, BRTHA3011265, BRTHA3011361, BRTHA3011510, BRTHA3011892, BRTHA3012265,

BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3021971, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556,

BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000252, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, CHONS2002829, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318, COLON2004351,

COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000009, CTONG1000020, CTONG1000022, CTONG1000040, CTONG1000056, CTONG1000062, CTONG1000086, CTONG1000087, CTONG1000088, CTONG1000093, CTONG1000097, CTONG1000113, CTONG1000137, CTONG1000165, CTONG1000175, CTONG1000180, CTONG1000181, CTONG1000220, CTONG1000241, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042,

CTONG2000063, CTONG2000201, CTONG2000218, CTONG2000279, CTONG2000303, CTONG2000330, CTONG2000395, CTONG2000411, CTONG2000452, CTONG2000465, CTONG2000469, CTONG2000480, CTONG2000508, CTONG2000561, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001366, CTONG2001413, CTONG2001428, CTONG2001513, CTONG2001524, CTONG2001533, CTONG2001598, CTONG2001681, CTONG2001724, CTONG2001748, CTONG2001749, CTONG2001800, CTONG2001820, CTONG2001858, CTONG2001877, CTONG2001911, CTONG2001932, CTONG2001955, CTONG2002066, CTONG2002073, CTONG2002095, CTONG2002143, CTONG2002270, CTONG2002395, CTONG2002417,

CTONG2002418, CTONG2002453, CTONG2002520, CTONG2002558, CTONG2002590, CTONG2002709, CTONG2002721, CTONG2002766, CTONG2002803, CTONG2002816, CTONG2002820, CTONG2002832, CTONG2002841, CTONG2002903, CTONG2002965, CTONG2002999, CTONG2003028, CTONG2003094, CTONG2003100, CTONG2003115, CTONG2003189, CTONG2003245, CTONG2003293, CTONG2003298, CTONG2003348, CTONG2003350, CTONG2003361, CTONG2003372, CTONG2003375, CTONG2003427, CTONG2003517, CTONG2003524, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004423, CTONG2004454, CTONG2004487,

CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005278, CTONG2005290, CTONG2005374, CTONG2005399, CTONG2005468, CTONG2005553, CTONG2005567, CTONG2005585, CTONG2005615, CTONG2005635, CTONG2005775, CTONG2005795, CTONG2005890, CTONG2005904, CTONG2005913, CTONG2006004, CTONG2006129, CTONG2006223, CTONG2006235, CTONG2006273, CTONG2006310, CTONG2006377, CTONG2006393, CTONG2006412, CTONG2006449, CTONG2006515, CTONG2006524, CTONG2006562, CTONG2006568, CTONG2006611, CTONG2006620, CTONG2006666, CTONG2006691, CTONG2006709, CTONG2006798, CTONG2006836, CTONG2006932, CTONG2006942, CTONG2006964,

CTONG2007007, CTONG2007009, CTONG2007072, CTONG2007078, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008269, CTONG2008321, CTONG2008343, CTONG2008398, CTONG2008402, CTONG2008405, CTONG2008466, CTONG2008506, CTONG2008518, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033,

CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010348, CTONG2010408, CTONG2010508, CTONG2010566, CTONG2010623, CTONG2010649, CTONG2010652, CTONG2010803, CTONG2010821, CTONG2011188,

CTONG2011429, CTONG2011474, CTONG2011676, CTONG2011716, CTONG2011765, CTONG2011779, CTONG2011801, CTONG2011815, CTONG2011822, CTONG2011825, CTONG2011920, CTONG2011985, CTONG2012001, CTONG2012029, CTONG2012050, CTONG2012077, CTONG2012101, CTONG2012123, CTONG2012158, CTONG2012230, CTONG2012401, CTONG2012422, CTONG2012425, CTONG2012447, CTONG2012453, CTONG2012473, CTONG2012476, CTONG2012533, CTONG2012554, CTONG2012564, CTONG2012607, CTONG2012661, CTONG2012724, CTONG2012738, CTONG2012745, CTONG2012758, CTONG2012843, CTONG2012847, CTONG2012879, CTONG2012901, CTONG2012996, CTONG2013128, CTONG2013149, CTONG2013156, CTONG2013178, CTONG2013222, CTONG2013284, CTONG2013339, CTONG2013348, CTONG2013352,

CTONG2013381, CTONG2013393, CTONG2013511, CTONG2013630, CTONG2013803, CTONG2013907, CTONG2013934, CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014369, CTONG2014389, CTONG2014491, CTONG2014630, CTONG2014635, CTONG2014646, CTONG2014705, CTONG2014898, CTONG2014946, CTONG2014954, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016056, CTONG2016217,

CTONG2016355, CTONG2016408, CTONG2016499, CTONG2016505, CTONG2016559, CTONG2016575, CTONG2016658, CTONG2016775, CTONG2016797, CTONG2016824, CTONG2016846, CTONG2016869, CTONG2016904, CTONG2016931, CTONG2016942, CTONG2016953, CTONG2017021, CTONG2017094, CTONG2017292, CTONG2017409, CTONG2017429, CTONG2017444, CTONG2017458, CTONG2017500, CTONG2017650, CTONG2017798, CTONG2017804, CTONG2017814, CTONG2017910, CTONG2017939, CTONG2017989, CTONG2017998, CTONG2018062, CTONG2018069, CTONG2018147, CTONG2018211, CTONG2018217, CTONG2018279, CTONG2018334, CTONG2018343, CTONG2018379, CTONG2018383, CTONG2018413, CTONG2018483, CTONG2018629, CTONG2018632, CTONG2018637, CTONG2018652, CTONG2018653, CTONG2018655,

CTONG2018735, CTONG2018739, CTONG2018808, CTONG2018843, CTONG2018898, CTONG2018900, CTONG2018976, CTONG2019029, CTONG2019063, CTONG2019144, CTONG2019232, CTONG2019248, CTONG2019400, CTONG2019590, CTONG2019652, CTONG2019704, CTONG2019822, CTONG2019833, CTONG2020031, CTONG2020108, CTONG2020127, CTONG2020171, CTONG2020374, CTONG2020378, CTONG2020411, CTONG2020445, CTONG2020484, CTONG2020522, CTONG2020560, CTONG2020582, CTONG2020638, CTONG2020806, CTONG2020831, CTONG2020974, CTONG2021024, CTONG2021132, CTONG2021168, CTONG2021289, CTONG2022153, CTONG2022601, CTONG2022835, CTONG2023021, CTONG2023449, CTONG2023512, CTONG2024031, CTONG2024206, CTONG2024614, CTONG2024749, CTONG2025496, CTONG2025513,

CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028309, CTONG2028486, CTONG2028518, CTONG2028687, CTONG2028758, CTONG3000657, CTONG3000686, CTONG3000707, CTONG3000896, CTONG3001123, CTONG3001420, CTONG3001501, CTONG3001560, CTONG3001605, CTONG3001746, CTONG3002020, CTONG3002127, CTONG3002370, CTONG3002433, CTONG3002518, CTONG3002552, CTONG3002588, CTONG3002674, CTONG3002728, CTONG3002835, CTONG3002909, CTONG3002947, CTONG3002983, CTONG3003165, CTONG3003179, CTONG3003483, CTONG3003553, CTONG3003598,

CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009227, CTONG3009239, CTONG3009287, CTONG3009328, CTONG3009385, D3OST1000066, D3OST1000109, D3OST1000238, D3OST1000267, D3OST1000270,

D3OST2000184, D3OST2000618, D3OST2000654, D3OST2000734, D3OST2001328, D3OST2001669, D3OST2001670, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000195, D3OST3000258, D3OST3000291, D3OST3000388, D3OST3000493, D6OST2000127, D6OST2000169, D6OST2000245, D6OST2000358, D6OST2000464, D6OST2000507, D9OST2000031, D9OST2000278, D9OST2000440, D9OST2000869, D9OST2001319, D9OST2001433, D9OST2002397, D9OST2002608, D9OST2002673, D9OST2003106, D9OST2003108, D9OST2003137, D9OST2003397, D9OST2003580, D9OST2003594, D9OST2003791, D9OST2003989, D9OST2004018,

D9OST2004073, D9OST2004417, DFNES1000003, DFNES1000083, DFNES1000107, DFNES1000150, DFNES1000185, DFNES2000011, DFNES2000143, DFNES2000146, DFNES2000153, DFNES2000212, DFNES2000292, DFNES2000335, DFNES2000426, DFNES2000432, DFNES2000457, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2002966, DFNES2003081, DFNES2003192, DFNES2003255, DFNES2003742, DFNES2004346, DFNES2004354, DFNES2004371, DFNES2004383, DFNES2004608, DFNES2004684, DFNES2005266, DFNES2005527, DFNES2005540, DFNES2005690,

DFNES2005766, DFNES2005779, DFNES2005875, DFNES2006346, DFNES2006944, DFNES2007113, DFNES2007299, DFNES2007332, DFNES2007634, DFNES2007641, DFNES2007757, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000023, FCBBF1000024, FCBBF1000025, FCBBF1000027, FCBBF1000038, FCBBF1000053, FCBBF1000061, FCBBF1000063, FCBBF1000069, FCBBF1000077, FCBBF1000115, FCBBF1000117, FCBBF1000118, FCBBF1000121, FCBBF1000125, FCBBF1000131, FCBBF1000139, FCBBF1000155, FCBBF1000157, FCBBF1000171, FCBBF1000174, FCBBF1000182, FCBBF1000197, FCBBF1000198,

FCBBF1000206, FCBBF1000220, FCBBF1000232, FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000367, FCBBF1000374, FCBBF1000376, FCBBF1000377, FCBBF1000395, FCBBF1000397, FCBBF1000403, FCBBF1000412, FCBBF1000425, FCBBF1000437, FCBBF1000449, FCBBF1000466, FCBBF1000476, FCBBF1000506, FCBBF1000509, FCBBF1000546, FCBBF1000550, FCBBF1000556, FCBBF1000563, FCBBF1000574, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF1000760, FCBBF2000038, FCBBF2000094,

FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566, FCBBF2000576, FCBBF2000591, FCBBF2000677, FCBBF2000678, FCBBF2000685, FCBBF2000733, FCBBF2000782, FCBBF2000808, FCBBF2000815, FCBBF2000825, FCBBF2000885, FCBBF2000940, FCBBF2000951, FCBBF2001001, FCBBF2001088, FCBBF2001105, FCBBF2001116, FCBBF2001183, FCBBF2001188, FCBBF2001211, FCBBF2001238, FCBBF2001243, FCBBF2001291, FCBBF2001299, FCBBF2001309, FCBBF2001400, FCBBF2001427, FCBBF2001480, FCBBF2001529, FCBBF2001538, FCBBF2001594, FCBBF2001672, FCBBF2001675, FCBBF2001718, FCBBF2001720, FCBBF2001817,

FCBBF2001868, FCBBF2002044, FCBBF2002111, FCBBF2002281, FCBBF2002349, FCBBF2002370, FCBBF2002541, FCBBF2002626, FCBBF2002801, FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005909, FCBBF2005966, FCBBF2006131, FCBBF2006380,

FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000110, FCBBF3000115, FCBBF3000120, FCBBF3000168, FCBBF3000175, FCBBF3000227, FCBBF3000228, FCBBF3000229, FCBBF3000233, FCBBF3000269, FCBBF3000361, FCBBF3000367, FCBBF3000388, FCBBF3000434, FCBBF3000462, FCBBF3000473, FCBBF3000501, FCBBF3000518, FCBBF3000536, FCBBF3000550, FCBBF3000847, FCBBF3001018, FCBBF3001020, FCBBF3001081, FCBBF3001235, FCBBF3001281, FCBBF3001302, FCBBF3001377, FCBBF3001594, FCBBF3001632, FCBBF3001657, FCBBF3001818, FCBBF3001855, FCBBF3001890, FCBBF3001912, FCBBF3001914, FCBBF3001918,

FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004261, FCBBF3004323, FCBBF3004390, FCBBF3004409, FCBBF3004473, FCBBF3004502, FCBBF3004715, FCBBF3004842, FCBBF3004847, FCBBF3004955, FCBBF3005160, FCBBF3005218, FCBBF3005320, FCBBF3005330, FCBBF3005444, FCBBF3005497, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152,

FCBBF3007242, FCBBF3007244, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008153, FCBBF3008159, FCBBF3008251, FCBBF3008311, FCBBF3008362, FCBBF3008382, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211,

FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809,

FCBBF3015870, FCBBF3016018, FCBBF3016134, FCBBF3016178, FCBBF3016451, FCBBF3016618, FCBBF3016622, FCBBF3016644, FCBBF3016667, FCBBF3016928, FCBBF3016987, FCBBF3017024, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018796, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019714, FCBBF3019716, FCBBF3019784, FCBBF3019839, FCBBF3019867, FCBBF3019961, FCBBF3020030, FCBBF3020138,

FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078, FCBBF3023368, FCBBF3023372, FCBBF3023443, FCBBF3023599, FCBBF3023667, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730,

FCBBF3025737, FCBBF3025905, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028244, FCBBF3028472, FCBBF3028528, FCBBF3028582, FCBBF3028593, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000446, FCBBF4000500, FCBBF4000529, FCBBF4000548, FCBBF5000041, FCBBF5000061, FCBBF5000165, FCBBF5000261, FCBBF5000325,

FCBBF5000353, FCBBF5000384, FCBBF5000483, FCBBF5000495, FEBRA1000005, FEBRA1000008, FEBRA1000022, FEBRA1000030, FEBRA1000057, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000105, FEBRA2000126, FEBRA2000129, FEBRA2000210, FEBRA2000220, FEBRA2000228, FEBRA2000253, FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000452, FEBRA2000454, FEBRA2000462, FEBRA2000468,

FEBRA2000491, FEBRA2000504, FEBRA2000510, FEBRA2000532, FEBRA2000536, FEBRA2000538, FEBRA2000575, FEBRA2000581, FEBRA2000656, FEBRA2000659, FEBRA2000680, FEBRA2000690, FEBRA2000727, FEBRA2000734, FEBRA2000740, FEBRA2000757, FEBRA2000762, FEBRA2000771, FEBRA2000772, FEBRA2000782, FEBRA2000787, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000881, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197,

FEBRA2001217, FEBRA2001227, FEBRA2001245, FEBRA2001267, FEBRA2001294, FEBRA2001334, FEBRA2001351, FEBRA2001383, FEBRA2001438, FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001752, FEBRA2001772, FEBRA2001790, FEBRA2001814, FEBRA2001828, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002429, FEBRA2002442, FEBRA2002456, FEBRA2002508, FEBRA2002525,

FEBRA2002527, FEBRA2002552, FEBRA2002574, FEBRA2002601, FEBRA2002611, FEBRA2002628, FEBRA2002662, FEBRA2002682, FEBRA2002707, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436, FEBRA2003454, FEBRA2003468, FEBRA2003470, FEBRA2003520, FEBRA2003524, FEBRA2003675, FEBRA2003707, FEBRA2003711, FEBRA2003750, FEBRA2003822, FEBRA2003833, FEBRA2003897, FEBRA2003907, FEBRA2003926, FEBRA2004023,

FEBRA2004026, FEBRA2004029, FEBRA2004042, FEBRA2004053, FEBRA2004056, FEBRA2004091, FEBRA2004110, FEBRA2004191, FEBRA2004202, FEBRA2004219, FEBRA2004224, FEBRA2004237, FEBRA2004268, FEBRA2004293, FEBRA2004325, FEBRA2004329, FEBRA2004331, FEBRA2004412, FEBRA2004443, FEBRA2004485, FEBRA2004490, FEBRA2004538, FEBRA2004592, FEBRA2004620, FEBRA2004628, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005416, FEBRA2005427, FEBRA2005458, FEBRA2005476, FEBRA2005572, FEBRA2005701,

FEBRA2005726, FEBRA2005752, FEBRA2005778, FEBRA2005787, FEBRA2005788, FEBRA2005824, FEBRA2005995, FEBRA2005998, FEBRA2006055, FEBRA2006061, FEBRA2006092, FEBRA2006112, FEBRA2006150, FEBRA2006165, FEBRA2006255, FEBRA2006270, FEBRA2006315, FEBRA2006346, FEBRA2006354, FEBRA2006357, FEBRA2006368, FEBRA2006370, FEBRA2006372, FEBRA2006379, FEBRA2006396, FEBRA2006401, FEBRA2006409, FEBRA2006418, FEBRA2006427, FEBRA2006476, FEBRA2006485, FEBRA2006491, FEBRA2006519, FEBRA2006521, FEBRA2006553, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017, FEBRA2007176, FEBRA2007200,

FEBRA2007212, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007620, FEBRA2007622, FEBRA2007708, FEBRA2007714, FEBRA2007793, FEBRA2007801, FEBRA2007818, FEBRA2007823, FEBRA2007880, FEBRA2007900, FEBRA2007901, FEBRA2007931, FEBRA2007957, FEBRA2008009, FEBRA2008036, FEBRA2008081, FEBRA2008086, FEBRA2008134, FEBRA2008135, FEBRA2008159, FEBRA2008165, FEBRA2008172, FEBRA2008201, FEBRA2008210, FEBRA2008255, FEBRA2008265, FEBRA2008266, FEBRA2008282, FEBRA2008287, FEBRA2008302, FEBRA2008311, FEBRA2008341, FEBRA2008360, FEBRA2008459, FEBRA2008468, FEBRA2008475, FEBRA2008481,

FEBRA2008542, FEBRA2008660, FEBRA2008662, FEBRA2008681, FEBRA2008692, FEBRA2008741, FEBRA2008755, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009419, FEBRA2009478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768,

FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013699, FEBRA2013905, FEBRA2013951, FEBRA2014010, FEBRA2014122, FEBRA2014198, FEBRA2014213, FEBRA2014297, FEBRA2014417, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223,

FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022055, FEBRA2022148, FEBRA2022204, FEBRA2022255,

FEBRA2022322, FEBRA2022504, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023377, FEBRA2023399, FEBRA2023498, FEBRA2023550, FEBRA2023721, FEBRA2023927, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025427, FEBRA2025463, FEBRA2025477, FEBRA2025838, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158,

FEBRA2028222, FEBRA2028366, FEBRA2028457, FEBRA2028477, FEBRA2028503, FEBRA2028516, FEHRT2000325, FEHRT2000364, FEKID1000018, FELIV1000101, FELIV1000137, FELIV1000164, FELNG2000241, HCASM1000021, HCASM1000050, HCASM1000061, HCASM1000115, HCASM1000130, HCASM2000016, HCASM2000138, HCASM2000156, HCASM2000202, HCASM2000214, HCASM2000266, HCASM2000307, HCASM2000363, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005388, HCASM2005484, HCASM2006131, HCASM2006338, HCASM2006359,

HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000015, HCHON1000030, HCHON1000131, HCHON1000142, HCHON1000166, HCHON1000176, HCHON1000191, HCHON2000028, HCHON2000038, HCHON2000062, HCHON2000100, HCHON2000156, HCHON2000160, HCHON2000199, HCHON2000212, HCHON2000226, HCHON2000244, HCHON2000265, HCHON2000271, HCHON2000315, HCHON2000322, HCHON2000323, HCHON2000344, HCHON2000364, HCHON2000373, HCHON2000395, HCHON2000417, HCHON2000418, HCHON2000473, HCHON2000475, HCHON2000503, HCHON2000508, HCHON2000626, HCHON2000660, HCHON2000663, HCHON2000673, HCHON2000676, HCHON2000698, HCHON2000699,

HCHON2000705, HCHON2000738, HCHON2000743, HCHON2000751, HCHON2000817, HCHON2000818, HCHON2000819, HCHON2000826, HCHON2000832, HCHON2000851, HCHON2000898, HCHON2000935, HCHON2000942, HCHON2000956, HCHON2000994, HCHON2001039, HCHON2001050, HCHON2001084, HCHON2001099, HCHON2001108, HCHON2001110, HCHON2001116, HCHON2001161, HCHON2001200, HCHON2001214, HCHON2001217, HCHON2001233, HCHON2001269, HCHON2001407, HCHON2001434, HCHON2001450, HCHON2001453, HCHON2001497, HCHON2001505, HCHON2001519, HCHON2001523, HCHON2001535, HCHON2001546, HCHON2001548, HCHON2001577, HCHON2001598, HCHON2001604, HCHON2001607, HCHON2001646, HCHON2001665, HCHON2001712, HCHON2001768, HCHON2001801, HCHON2001853, HCHON2002123,

HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005921, HCHON2005987, HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871,

HCHON2008989, HCHON2009191, HCHON2009384, HCHON2009749, HCHON2009766, HCHON2009795, HCHON2010074, HCHON2010543, HEART1000010, HEART1000074, HEART1000088, HEART1000099, HEART1000102, HEART1000118, HEART1000132, HEART1000139, HEART1000142, HEART1000149, HEART1000151, HEART1000185, HEART2000024, HEART2000035, HEART2000099, HEART2000298, HEART2000306, HEART2000309, HEART2000411, HEART2000418, HEART2000444, HEART2000448, HEART2000492, HEART2000506, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440,

HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006521, HEART2006557, HEART2006787, HEART2006789, HEART2006909, HEART2007031, HEART2007231, HEART2007443, HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC1000083, HHDPC1000118, HHDPC1000163, HHDPC2000055, HHDPC2000070, HHDPC2000102, HHDPC2000115,

HHDPC2000149, HHDPC2000258, HHDPC2000315, HHDPC2000455, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006862, HHDPC2006927, HHDPC2007267, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030, HLUNG1000034, HLUNG1000037, HLUNG1000053, HLUNG1000055, HLUNG1000064, HLUNG1000076, HLUNG1000084,

HLUNG1000091, HLUNG1000099, HLUNG1000104, HLUNG1000105, HLUNG1000110, HLUNG1000151, HLUNG1000169, HLUNG2000004, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363, HLUNG2000412, HLUNG2000431, HLUNG2000480, HLUNG2000489, HLUNG2000549, HLUNG2000557, HLUNG2000570, HLUNG2000751, HLUNG2000761, HLUNG2000777, HLUNG2000846, HLUNG2000870, HLUNG2000884, HLUNG2000926, HLUNG2000950, HLUNG2000955, HLUNG2000970, HLUNG2001013, HLUNG2001126, HLUNG2001144, HLUNG2001146, HLUNG2001214, HLUNG2001459,

HLUNG2001507, HLUNG2001518, HLUNG2001633, HLUNG2001677, HLUNG2001712, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002562, HLUNG2002648, HLUNG2002659, HLUNG2002707, HLUNG2002811, HLUNG2002916, HLUNG2002942, HLUNG2002949, HLUNG2002958, HLUNG2002982, HLUNG2003003, HLUNG2003042, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003833, HLUNG2003872, HLUNG2003918, HLUNG2003991, HLUNG2004014, HLUNG2004037, HLUNG2004067, HLUNG2004154,

HLUNG2004159, HLUNG2004170, HLUNG2004217, HLUNG2004273, HLUNG2004388, HLUNG2004416, HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006812, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008139, HLUNG2008153,

HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008637, HLUNG2008833, HLUNG2008875, HLUNG2008885, HLUNG2008958, HLUNG2008959, HLUNG2009067, HLUNG2009215, HLUNG2009225, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013851, HLUNG2014262, HLUNG2014288, HLUNG2014449, HLUNG2014821, HLUNG2015184,

HLUNG2015418, HLUNG2015548, HLUNG2015578, HLUNG2015617, HLUNG2016022, HLUNG2016862, HLUNG2017262, HLUNG2017286, HLUNG2017307, HLUNG2017350, HLUNG2017806, HLUNG2018029, HLUNG2018282, HLUNG2018626, HLUNG2019058, HSYRA1000062, HSYRA1000131, HSYRA1000137, HSYRA1000148, HSYRA1000152, HSYRA1000178, HSYRA2000044, HSYRA2000135, HSYRA2000137, HSYRA2000190, HSYRA2000221, HSYRA2000224, HSYRA2000232, HSYRA2000237, HSYRA2000248, HSYRA2000253, HSYRA2000255, HSYRA2000332, HSYRA2000371, HSYRA2000424, HSYRA2000507, HSYRA2000510, HSYRA2000605, HSYRA2000629, HSYRA2000640, HSYRA2000706, HSYRA2000743, HSYRA2000760, HSYRA2000787, HSYRA2000792, HSYRA2000828, HSYRA2000832, HSYRA2000925, HSYRA2000927, HSYRA2001003,

HSYRA2001103, HSYRA2001105, HSYRA2001138, HSYRA2001142, HSYRA2001153, HSYRA2001232, HSYRA2001255, HSYRA2001332, HSYRA2001353, HSYRA2001396, HSYRA2001420, HSYRA2001484, HSYRA2001552, HSYRA2001567, HSYRA2001574, HSYRA2001580, HSYRA2001615, HSYRA2001631, HSYRA2001638, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000158, IMR321000161, IMR321000165, IMR321000193, IMR321000207, IMR321000230, IMR321000242, IMR321000266, IMR322000010, IMR322000072,

IMR322000107, IMR322000127, IMR322000144, IMR322000152, IMR322000164, IMR322000178, IMR322000243, IMR322000302, IMR322000316, IMR322000374, IMR322000379, IMR322000383, IMR322000418, IMR322000518, IMR322000555, IMR322000604, IMR322000609, IMR322000656, IMR322000672, IMR322000730, IMR322000742, IMR322000775, IMR322000791, IMR322000811, IMR322000819, IMR322000838, IMR322000859, IMR322000863, IMR322000917, IMR322000918, IMR322000919, IMR322000935, IMR322000953, IMR322000973, IMR322000984, IMR322001018, IMR322001049, IMR322001167, IMR322001185, IMR322001245, IMR322001317, IMR322001318, IMR322001322, IMR322001332, IMR322001380, IMR322001435, IMR322001438, IMR322001452, IMR322001491, IMR322001534,

IMR322001541, IMR322001584, IMR322001665, IMR322001670, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, IMR322019070, JCMLC2000273, KIDNE1000008, KIDNE1000012, KIDNE1000015, KIDNE1000028, KIDNE1000036, KIDNE1000064, KIDNE1000104,

KIDNE1000121, KIDNE1000143, KIDNE1000145, KIDNE1000152, KIDNE2000026, KIDNE2000041, KIDNE2000046, KIDNE2000051, KIDNE2000070, KIDNE2000085, KIDNE2000167, KIDNE2000174, KIDNE2000192, KIDNE2000227, KIDNE2000244, KIDNE2000245, KIDNE2000252, KIDNE2000266, KIDNE2000287, KIDNE2000315, KIDNE2000317, KIDNE2000328, KIDNE2000330, KIDNE2000335, KIDNE2000349, KIDNE2000375, KIDNE2000383, KIDNE2000394, KIDNE2000403, KIDNE2000422, KIDNE2000493, KIDNE2000497, KIDNE2000510, KIDNE2000513, KIDNE2000517, KIDNE2000519, KIDNE2000555, KIDNE2000574, KIDNE2000624, KIDNE2000665, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867,

KIDNE2000909, KIDNE2000947, KIDNE2001117, KIDNE2001140, KIDNE2001162, KIDNE2001269, KIDNE2001361, KIDNE2001373, KIDNE2001636, KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002252, KIDNE2002262, KIDNE2002265, KIDNE2002438, KIDNE2002483, KIDNE2002725, KIDNE2002768, KIDNE2002795, KIDNE2002798, KIDNE2002839, KIDNE2002845, KIDNE2002872, KIDNE2002882, KIDNE2002883, KIDNE2002980, KIDNE2002991, KIDNE2003185, KIDNE2003188, KIDNE2003233, KIDNE2003305, KIDNE2003316, KIDNE2003335, KIDNE2003357, KIDNE2003373, KIDNE2003377, KIDNE2003507, KIDNE2003752,

KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411, KIDNE2004475, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005676, KIDNE2005781,

KIDNE2005798, KIDNE2005854, KIDNE2005908, KIDNE2005937, KIDNE2006009, KIDNE2006014, KIDNE2006030, KIDNE2006039, KIDNE2006053, KIDNE2006062, KIDNE2006092, KIDNE2006145, KIDNE2006149, KIDNE2006210, KIDNE2006248, KIDNE2006299, KIDNE2006334, KIDNE2006353, KIDNE2006376, KIDNE2006378, KIDNE2006465, KIDNE2006580, KIDNE2006652, KIDNE2006687, KIDNE2006733, KIDNE2006760, KIDNE2006775, KIDNE2006811, KIDNE2006820, KIDNE2006880, KIDNE2007005, KIDNE2007040, KIDNE2007077, KIDNE2007186, KIDNE2007222, KIDNE2007356, KIDNE2007422, KIDNE2007569, KIDNE2007810, KIDNE2007811, KIDNE2007910, KIDNE2007954, KIDNE2008022, KIDNE2008048, KIDNE2008069, KIDNE2008072, KIDNE2008116, KIDNE2008117, KIDNE2008218, KIDNE2008315,

KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009367, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750, KIDNE2010762,

KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013845, KIDNE2013866, KIDNE2014087, KIDNE2014112, KIDNE2014119, KIDNE2014170, KIDNE2014184, KIDNE2014268, KIDNE2014298, KIDNE2014320,

KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539, KIDNE2016570, KIDNE2016918, KIDNE2017007, KIDNE2017040, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166,

KIDNE2018167, KIDNE2018217, KIDNE2018254, KIDNE2018268, KIDNE2018269, KIDNE2018287, KIDNE2018294, KIDNE2018352, KIDNE2018462, KIDNE2018493, KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000079, LIVER1000082, LIVER1000099, LIVER1000104, LIVER1000111, LIVER1000126, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000033, LIVER2000037, LIVER2000158,

LIVER2000216, LIVER2000237, LIVER2000247, LIVER2000360, LIVER2000416, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2001608, LIVER2001835, LIVER2002638, LIVER2002644, LIVER2002660, LIVER2002702, LIVER2002842, LIVER2003008, LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520,

LIVER2005527, LIVER2005544, LIVER2005625, LIVER2005728, LIVER2005789, LIVER2005973, LIVER2005981, LIVER2006006, LIVER2006251, LIVER2006410, LIVER2006469, LIVER2006644, LIVER2006764, LIVER2007415, LIVER2007548, LIVER2007568, LIVER2007721, LIVER2007783, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000035, MAMGL1000083, MAMGL1000096, MAMGL1000100, MAMGL1000132, MAMGL1000151, MAMGL1000173, MAMGL1000178, MAMGL1000182, MAMGL1000184, MESAN1000032, MESAN1000035, MESAN1000050, MESAN1000079, MESAN1000080, MESAN1000101, MESAN1000121,

MESAN1000126, MESAN1000147, MESAN1000180, MESAN2000023, MESAN2000026, MESAN2000067, MESAN2000092, MESAN2000149, MESAN2000167, MESAN2000264, MESAN2000291, MESAN2000337, MESAN2000434, MESAN2000457, MESAN2000462, MESAN2000501, MESAN2000572, MESAN2000608, MESAN2000620, MESAN2000711, MESAN2000815, MESAN2000894, MESAN2000909, MESAN2001154, MESAN2001450, MESAN2001627, MESAN2001770, MESAN2001979, MESAN2001980, MESAN2002086, MESAN2002113, MESAN2002122, MESAN2002147, MESAN2002186, MESAN2002424, MESAN2002519, MESAN2002687, MESAN2002709, MESAN2002790, MESAN2002844, MESAN2002940, MESAN2002978, MESAN2003035, MESAN2003037, MESAN2003039, MESAN2003058, MESAN2003101, MESAN2003190, MESAN2003322, MESAN2003444,

MESAN2003490, MESAN2003529, MESAN2003646, MESAN2003662, MESAN2003709, MESAN2003851, MESAN2004138, MESAN2004288, MESAN2004575, MESAN2005284, MESAN2005303, MESAN2005371, MESAN2005633, MESAN2005689, MESAN2005724, MESAN2005766, MESAN2005808, MESAN2005811, MESAN2005957, MESAN2005958, MESAN2006022, MESAN2006043, MESAN2006328, MESAN2006401, MESAN2006580, MESAN2006599, MESAN2006743, MESAN2006953, MESAN2006971, MESAN2007032, MESAN2007440, MESAN2007597, MESAN2007755, MESAN2008222, MESAN2008334, MESAN2008415, MESAN2008460, MESAN2008536, MESAN2008679, MESAN2008729, MESAN2008821, MESAN2008926, MESAN2008936, MESAN2009019, MESAN2009081, MESAN2009105, MESAN2009311, MESAN2009418, MESAN2009503, MESAN2009522,

MESAN2009580, MESAN2010031, MESAN2010114, MESAN2010312, MESAN2010321, MESAN2010664, MESAN2010681, MESAN2011545, MESAN2011597, MESAN2011627, MESAN2011632, MESAN2011977, MESAN2011984, MESAN2012054, MESAN2012113, MESAN2012586, MESAN2012708, MESAN2012735, MESAN2013019, MESAN2013022, MESAN2013183, MESAN2013211, MESAN2013283, MESAN2013284, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016552, MESAN2016965, MESAN2017133, MESAN2017152, MESAN2017417, MESAN2018209,

MESAN2018576, MESAN2018670, MESAN2018699, MESTC1000042, MESTC1000147, MESTC2000150, MESTC2000153, MESTC2000170, N1ESE2000698, NB9N41000011, NB9N41000047, NB9N41000121, NB9N41000135, NB9N41000142, NB9N41000146, NB9N41000340, NB9N42000104, NB9N42000122, NB9N42000196, NB9N42000281, NB9N42000314, NB9N42000430, NB9N42000495, NB9N42001300, NT2NE1000004, NT2NE1000014, NT2NE1000018, NT2NE1000023, NT2NE1000024, NT2NE1000059, NT2NE1000063, NT2NE1000073, NT2NE1000083, NT2NE1000120, NT2NE1000122, NT2NE1000163, NT2NE1000175, NT2NE1000181, NT2NE1000185, NT2NE2000038, NT2NE2000054, NT2NE2000056, NT2NE2000057, NT2NE2000064, NT2NE2000109, NT2NE2000137, NT2NE2000156, NT2NE2000174, NT2NE2000195, NT2NE2000222,

NT2NE2000259, NT2NE2000284, NT2NE2000299, NT2NE2000327, NT2NE2000353, NT2NE2000356, NT2NE2000369, NT2NE2000374, NT2NE2000383, NT2NE2000384, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000586, NT2NE2000612, NT2NE2000636, NT2NE2000658, NT2NE2000706, NT2NE2000707, NT2NE2000763, NT2NE2000802, NT2NE2000808, NT2NE2000809, NT2NE2000864, NT2NE2000909, NT2NE2000913, NT2NE2000950, NT2NE2000951, NT2NE2000980, NT2NE2001005, NT2NE2001021, NT2NE2001040, NT2NE2001048, NT2NE2001049, NT2NE2001142, NT2NE2001156, NT2NE2001176, NT2NE2001180, NT2NE2001181, NT2NE2001188, NT2NE2001191, NT2NE2001247,

NT2NE2001252, NT2NE2001324, NT2NE2001326, NT2NE2001337, NT2NE2001364, NT2NE2001372, NT2NE2001397, NT2NE2001403, NT2NE2001428, NT2NE2001432, NT2NE2001435, NT2NE2001442, NT2NE2001524, NT2NE2001530, NT2NE2001545, NT2NE2001598, NT2NE2001617, NT2NE2001623, NT2NE2001626, NT2NE2001634, NT2NE2001648, NT2NE2001655, NT2NE2001660, NT2NE2001666, NT2NE2001677, NT2NE2001697, NT2NE2001698, NT2NE2001723, NT2NE2001725, NT2NE2001793, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2003150, NT2NE2003185, NT2NE2003252, NT2NE2003280, NT2NE2003308, NT2NE2003309,

NT2NE2003315, NT2NE2003393, NT2NE2003408, NT2NE2003457, NT2NE2003485, NT2NE2003540, NT2NE2003569, NT2NE2003571, NT2NE2003705, NT2NE2003752, NT2NE2003887, NT2NE2003921, NT2NE2004255, NT2NE2004378, NT2NE2004490, NT2NE2004519, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005419, NT2NE2005441, NT2NE2005517, NT2NE2005537, NT2NE2005555, NT2NE2005588, NT2NE2005676, NT2NE2005688, NT2NE2005720, NT2NE2005784, NT2NE2005804, NT2NE2005821, NT2NE2005876, NT2NE2005890, NT2NE2005921, NT2NE2005968, NT2NE2006075, NT2NE2006087, NT2NE2006103, NT2NE2006217, NT2NE2006288,

NT2NE2006382, NT2NE2006387, NT2NE2006427, NT2NE2006432, NT2NE2006458, NT2NE2006478, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654,

NT2NE2010688, NT2NE2010781, NT2NE2010842, NT2NE2010854, NT2NE2011033, NT2NE2011036, NT2NE2011107, NT2NE2011119, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014104, NT2NE2014113, NT2NE2014176, NT2NE2014221, NT2NE2014525, NT2NE2014650, NT2NE2014651, NT2NE2014681, NT2NE2014758,

NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018594, NT2NE2018739, NT2NE2018916, NT2NE2019056, NT2NE2019076, NT2NE2019091, NT2NE2019099,

NT2RI1000016, NT2RI1000027, NT2RI1000035, NT2RI1000048, NT2RI1000127, NT2RI1000164, NT2RI1000172, NT2RI2000004, NT2RI2000007, NT2RI2000064, NT2RI2000107, NT2RI2000116, NT2RI2000117, NT2RI2000128, NT2RI2000133, NT2RI2000167, NT2RI2000173, NT2RI2000187, NT2RI2000255, NT2RI2000263, NT2RI2000270, NT2RI2000276, NT2RI2000282, NT2RI2000284, NT2RI2000294, NT2RI2000313, NT2RI2000341, NT2RI2000344, NT2RI2000348, NT2RI2000412, NT2RI2000480, NT2RI2000575, NT2RI2000578, NT2RI2000588, NT2RI2000597, NT2RI2000669, NT2RI2000671, NT2RI2000685, NT2RI2000688, NT2RI2000689, NT2RI2000704, NT2RI2000738, NT2RI2000775, NT2RI2000822, NT2RI2000829, NT2RI2000841, NT2RI2000865, NT2RI2000932, NT2RI2000974, NT2RI2000987,

NT2RI2001010, NT2RI2001017, NT2RI2001030, NT2RI2001054, NT2RI2001083, NT2RI2001091, NT2RI2001167, NT2RI2001178, NT2RI2001235, NT2RI2001244, NT2RI2001307, NT2RI2001385, NT2RI2001409, NT2RI2001410, NT2RI2001422, NT2RI2001425, NT2RI2001449, NT2RI2001450, NT2RI2001474, NT2RI2001519, NT2RI2001595, NT2RI2001621, NT2RI2001624, NT2RI2001657, NT2RI2001726, NT2RI2001731, NT2RI2001769, NT2RI2001782, NT2RI2001836, NT2RI2001846, NT2RI2001859, NT2RI2001866, NT2RI2001910, NT2RI2001952, NT2RI2002022, NT2RI2002041, NT2RI2002117, NT2RI2002120, NT2RI2002152, NT2RI2002153, NT2RI2002179, NT2RI2002243, NT2RI2002252, NT2RI2002260, NT2RI2002266, NT2RI2002270, NT2RI2002334, NT2RI2002359, NT2RI2002391, NT2RI2002447,

NT2RI2002481, NT2RI2002507, NT2RI2002517, NT2RI2002530, NT2RI2002540, NT2RI2002549, NT2RI2002554, NT2RI2002564, NT2RI2002585, NT2RI2002592, NT2RI2002602, NT2RI2002654, NT2RI2002699, NT2RI2002729, NT2RI2002802, NT2RI2002819, NT2RI2002822, NT2RI2002847, NT2RI2002852, NT2RI2002865, NT2RI2002923, NT2RI2002926, NT2RI2002958, NT2RI2002970, NT2RI2003011, NT2RI2003019, NT2RI2003051, NT2RI2003067, NT2RI2003184, NT2RI2003205, NT2RI2003222, NT2RI2003301, NT2RI2003317, NT2RI2003338, NT2RI2003344, NT2RI2003360, NT2RI2003361, NT2RI2003383, NT2RI2003392, NT2RI2003399, NT2RI2003407, NT2RI2003419, NT2RI2003420, NT2RI2003655, NT2RI2003667, NT2RI2003678, NT2RI2003751, NT2RI2003884, NT2RI2003921, NT2RI2003973,

NT2RI2003993, NT2RI200405.9, NT2RI2004078, NT2RI2004079, NT2RI2004093, NT2RI2004136, NT2RI2004157, NT2RI2004188, NT2RI2004190, NT2RI2004209, NT2RI2004230, NT2RI2004290, NT2RI2004304, NT2RI2004312, NT2RI2004381, NT2RI2004398, NT2RI2004410, NT2RI2004442, NT2RI2004468, NT2RI2004535, NT2RI2004606, NT2RI2004608, NT2RI2004618, NT2RI2004783, NT2RI2004818, NT2RI2004840, NT2RI2004884, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005116, NT2RI2005130, NT2RI2005166, NT2RI2005239, NT2RI2005246, NT2RI2005315, NT2RI2005335, NT2RI2005353, NT2RI2005358, NT2RI2005382, NT2RI2005405, NT2RI2005473, NT2RI2005520,

NT2RI2005564, NT2RI2005579, NT2RI2005621, NT2RI2005628, NT2RI2005647, NT2RI2005670, NT2RI2005710, NT2RI2005713, NT2RI2005723, NT2RI2005772, NT2RI2005811, NT2RI2005816, NT2RI2005818, NT2RI2005851, NT2RI2005966, NT2RI2006070, NT2RI2006071, NT2RI2006081, NT2RI2006126, NT2RI2006183, NT2RI2006257, NT2RI2006294, NT2RI2006345, NT2RI2006403, NT2RI2006412, NT2RI2006445, NT2RI2006487, NT2RI2006506, NT2RI2006553, NT2RI2006565, NT2RI2006640, NT2RI2006647, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006825, NT2RI2006838, NT2RI2006853, NT2RI2006855, NT2RI2006943, NT2RI2006973, NT2RI2007046,

NT2RI2007048, NT2RI2007054, NT2RI2007096, NT2RI2007116, NT2RI2007214, NT2RI2007277, NT2RI2007303, NT2RI2007334, NT2RI2007377, NT2RI2007386, NT2RI2007406, NT2RI2007439, NT2RI2007445, NT2RI2007468, NT2RI2007469, NT2RI2007498, NT2RI2007507, NT2RI2007572, NT2RI2007589, NT2RI2007593, NT2RI2007629, NT2RI2007723, NT2RI2007729, NT2RI2007751, NT2RI2007754, NT2RI2007827, NT2RI2007853, NT2RI2007877, NT2RI2007891, NT2RI2007956, NT2RI2007965, NT2RI2007987, NT2RI2008007, NT2RI2008045, NT2RI2008050, NT2RI2008141, NT2RI2008171, NT2RI2008188, NT2RI2008204, NT2RI2008221, NT2RI2008233, NT2RI2008336, NT2RI2008396, NT2RI2008455, NT2RI2008481, NT2RI2008502, NT2RI2008526, NT2RI2008566, NT2RI2008598, NT2RI2008656,

NT2RI2008714, NT2RI2008791, NT2RI2008801, NT2RI2008808, NT2RI2008812, NT2RI2008841, NT2RI2008942, NT2RI2008952, NT2RI2009005, NT2RI2009037, NT2RI2009065, NT2RI2009066, NT2RI2009083, NT2RI2009101, NT2RI2009103, NT2RI2009144, NT2RI2009151, NT2RI2009173, NT2RI2009215, NT2RI2009233, NT2RI2009239, NT2RI2009259, NT2RI2009269, NT2RI2009289, NT2RI2009301, NT2RI2009402, NT2RI2009406, NT2RI2009517, NT2RI2009583, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012350, NT2RI2012659, NT2RI2012726, NT2RI2012990, NT2RI2013345, NT2RI2013357, NT2RI2013373, NT2RI2014247, NT2RI2014551, NT2RI2014683, NT2RI2014733,

NT2RI2015239, NT2RI2015342, NT2RI2015533, NT2RI2016128, NT2RI2016134, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263,

NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006284, NT2RI3006340, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443, NT2RI3007543, NT2RI3007684, NT2RI3007757, NT2RI3007878, NT2RI3007978,

NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000008, NT2RP6000032, NT2RP6000033, NT2RP6000039, NT2RP6000043, NT2RP6000059, NT2RP6000060, NT2RP6000072, NT2RP6000077, NT2RP6000085, NT2RP6000086, NT2RP6000100, NT2RP6000109, NT2RP6000110, NT2RP6000119, NT2RP6000123, NT2RP6000127, NT2RP6000130, NT2RP6000140, NT2RP6000150, NT2RP7000041, NT2RP7000069, NT2RP7000086, NT2RP7000109, NT2RP7000112, NT2RP7000238, NT2RP7000259, NT2RP7000271, NT2RP7000311, NT2RP7000359, NT2RP7000364, NT2RP7000380, NT2RP7000391, NT2RP7000425, NT2RP7000477, NT2RP7000579, NT2RP7000586, NT2RP7000600, NT2RP7000624,

NT2RP7000812, NT2RP7000906, NT2RP7000926, NT2RP7001074, NT2RP7001080, NT2RP7001156, NT2RP7001166, NT2RP7001231, NT2RP7001283, NT2RP7001306, NT2RP7001319, NT2RP7001335, NT2RP7001364, NT2RP7001475, NT2RP7001532, NT2RP7001591, NT2RP7001602, NT2RP7001678, NT2RP7001962, NT2RP7002028, NT2RP7002064, NT2RP7002151, NT2RP7002163, NT2RP7002243, NT2RP7002282, NT2RP7002376, NT2RP7002379, NT2RP7002449, NT2RP7002450, NT2RP7002478, NT2RP7002554, NT2RP7002594, NT2RP7002603, NT2RP7002619, NT2RP7002650, NT2RP7002738, NT2RP7002779, NT2RP7002802, NT2RP7002829, NT2RP7002841, NT2RP7002875, NT2RP7002906, NT2RP7002978, NT2RP7002982, NT2RP7003025, NT2RP7003050, NT2RP7003055, NT2RP7003084, NT2RP7003091, NT2RP7003107,

NT2RP7003134, NT2RP7003148, NT2RP7003203, NT2RP7003261, NT2RP7003319, NT2RP7003439, NT2RP7003511, NT2RP7003629, NT2RP7003632, NT2RP7003647, NT2RP7003680, NT2RP7003688, NT2RP7003724, NT2RP7003960, NT2RP7004027, NT2RP7004080, NT2RP7004114, NT2RP7004123, NT2RP7004173, NT2RP7004196, NT2RP7004204, NT2RP7004233, NT2RP7004260, NT2RP7004348, NT2RP7004352, NT2RP7004358, NT2RP7004373, NT2RP7004396, NT2RP7004428, NT2RP7004481, NT2RP7004541, NT2RP7004559, NT2RP7004641, NT2RP7004656, NT2RP7004687, NT2RP7004722, NT2RP7004728, NT2RP7004751, NT2RP7004766, NT2RP7004790, NT2RP7004884, NT2RP7004911, NT2RP7004915, NT2RP7004925, NT2RP7004946, NT2RP7004961, NT2RP7004964, NT2RP7004975, NT2RP7005118, NT2RP7005205,

NT2RP7005219, NT2RP7005323, NT2RP7005468, NT2RP7005493, NT2RP7005513, NT2RP7005529, NT2RP7005629, NT2RP7005631, NT2RP7005669, NT2RP7005675, NT2RP7005750, NT2RP7005846, NT2RP7006033, NT2RP7006075, NT2RP7006141, NT2RP7006160, NT2RP7006162, NT2RP7006188, NT2RP7006223, NT2RP7006263, NT2RP7006296, NT2RP7006304, NT2RP7006374, NT2RP7006395, NT2RP7006408, NT2RP7006457, NT2RP7006539, NT2RP7006547, NT2RP7006601, NT2RP7006619, NT2RP7006621, NT2RP7006636, NT2RP7006701, NT2RP7006853, NT2RP7006886, NT2RP7006887, NT2RP7006980, NT2RP7006986, NT2RP7006995, NT2RP7007114, NT2RP7007154, NT2RP7007221, NT2RP7007226, NT2RP7007252, NT2RP7007269, NT2RP7007310, NT2RP7007359, NT2RP7007381, NT2RP7007387, NT2RP7007406,

NT2RP7007480, NT2RP7007504, NT2RP7007537, NT2RP7007580, NT2RP7007594, NT2RP7007610, NT2RP7007617, NT2RP7007643, NT2RP7007766, NT2RP7007842, NT2RP7007925, NT2RP7007930, NT2RP7007975, NT2RP7008013, NT2RP7008015, NT2RP7008133, NT2RP7008137, NT2RP7008142, NT2RP7008144, NT2RP7008161, NT2RP7008167, NT2RP7008315, NT2RP7008360, NT2RP7008396, NT2RP7008406, NT2RP7008435, NT2RP7008441, NT2RP7008454, NT2RP7008487, NT2RP7008543, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009087, NT2RP7009097, NT2RP7009108, NT2RP7009149, NT2RP7009168, NT2RP7009215, NT2RP7009225, NT2RP7009236,

NT2RP7009253, NT2RP7009259, NT2RP7009267, NT2RP7009322, NT2RP7009363, NT2RP7009370, NT2RP7009373, NT2RP7009394, NT2RP7009397, NT2RP7009429, NT2RP7009439, NT2RP7009466, NT2RP7009481, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014005, NT2RP7014178, NT2RP7014348, NT2RP7014420, NT2RP7014583,

NT2RP7014721, NT2RP7014743, NT2RP7014778, NT2RP7014906, NT2RP7014910, NT2RP7015080, NT2RP7015431, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633,

NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000033, NTONG1000047, NTONG1000052, NTONG1000075, NTONG1000087, NTONG1000098, NTONG1000123, NTONG1000130, NTONG1000161, NTONG1000172, NTONG1000182, NTONG1000213, NTONG1000214, NTONG1000247, NTONG1000264, NTONG2000040, NTONG2000107, NTONG2000231, NTONG2000265, NTONG2000363, NTONG2000413, NTONG2000492, NTONG2000499, NTONG2000531, NTONG2000583, NTONG2000858, NTONG2000876, NTONG2000878, NTONG2000966, NTONG2000977, NTONG2000985,

NTONG2001014, NTONG2001079, NTONG2001137, NTONG2001157, NTONG2001173, NTONG2001428, NTONG2001532, NTONG2001550, NTONG2001567, NTONG2001612, NTONG2001619, NTONG2001642, NTONG2001657, NTONG2001676, NTONG2001762, NTONG2002278, NTONG2002582, NTONG2002807, NTONG2002948, NTONG2002970, NTONG2002985, NTONG2003158, NTONG2003210, NTONG2003316, NTONG2003409, NTONG2003454, NTONG2003515, NTONG2003805, NTONG2003839, NTONG2003852, NTONG2003897, NTONG2003928, NTONG2004095, NTONG2004308, NTONG2004521, NTONG2004614, NTONG2004690, NTONG2004806, NTONG2004829, NTONG2004918, NTONG2004991, NTONG2005062, NTONG2005086, NTONG2005137, NTONG2005153, NTONG2005265, NTONG2005277, NTONG2005363, NTONG2005373, NTONG2005391,

NTONG2005480, NTONG2005497, NTONG2005520, NTONG2005577, NTONG2005657, NTONG2005801, NTONG2005822, NTONG2005897, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008483, NTONG2008522, NTONG2008672, NTONG2008862, NTONG2008939, NTONG2008944, NTONG2009060, NTONG2009068, NTONG2009229, NTONG2009233, NTONG2009468, NTONG2009576, OCBBF1000016, OCBBF1000030, OCBBF1000042, OCBBF1000049, OCBBF1000054,

OCBBF1000067, OCBBF1000080, OCBBF1000085, OCBBF1000086, OCBBF1000091, OCBBF1000104, OCBBF1000114, OCBBF1000118, OCBBF1000119, OCBBF1000122, OCBBF1000130, OCBBF1000138, OCBBF1000145, OCBBF1000146, OCBBF1000153, OCBBF1000175, OCBBF1000185, OCBBF1000188, OCBBF1000192, OCBBF1000254, OCBBF1000315, OCBBF2000013, OCBBF2000015, OCBBF2000126, OCBBF2000178, OCBBF2000231, OCBBF2000277, OCBBF2000287, OCBBF2000323, OCBBF2000452, OCBBF2000467, OCBBF2000522, OCBBF2000677, OCBBF2000703, OCBBF2000712, OCBBF2000719, OCBBF2000824, OCBBF2000831, OCBBF2000904, OCBBF2000986, OCBBF2000998, OCBBF2001075, OCBBF2001101, OCBBF2001124, OCBBF2001140, OCBBF2001166, OCBBF2001176, OCBBF2001186, OCBBF2001196, OCBBF2001210,

OCBBF2001252, OCBBF2001307, OCBBF2001323, OCBBF2001389, OCBBF2001402, OCBBF2001408, OCBBF2001416, OCBBF2001473, OCBBF2001492, OCBBF2001494, OCBBF2001527, OCBBF2001528, OCBBF2001586, OCBBF2001639, OCBBF2001681, OCBBF2001687, OCBBF2001706, OCBBF2001749, OCBBF2001794, OCBBF2001910, OCBBF2001931, OCBBF2001961, OCBBF2001983, OCBBF2002015, OCBBF2002083, OCBBF2002124, OCBBF2002161, OCBBF2002290, OCBBF2002357, OCBBF2002376, OCBBF2002615, OCBBF2002626, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003543, OCBBF2003593, OCBBF2003744, OCBBF2003819, OCBBF2004038, OCBBF2004104, OCBBF2004147,

OCBBF2004168, OCBBF2004235, OCBBF2004259, OCBBF2004273, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005433, OCBBF2005476, OCBBF2005546, OCBBF2005843, OCBBF2005901, OCBBF2005956, OCBBF2006005, OCBBF2006030, OCBBF2006058, OCBBF2006113, OCBBF2006148, OCBBF2006151, OCBBF2006154, OCBBF2006172, OCBBF2006214, OCBBF2006241, OCBBF2006388, OCBBF2006438, OCBBF2006567, OCBBF2006624, OCBBF2006639, OCBBF2006660, OCBBF2006764,

OCBBF2006849, OCBBF2006859, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007414, OCBBF2007428, OCBBF2007478, OCBBF2007485, OCBBF2007520, OCBBF2007555, OCBBF2007610, OCBBF2007622, OCBBF2007756, OCBBF2007762, OCBBF2007829, OCBBF2007892, OCBBF2007931, OCBBF2007946, OCBBF2008005, OCBBF2008116, OCBBF2008138, OCBBF2008144, OCBBF2008283, OCBBF2008330, OCBBF2008340, OCBBF2008366, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770,

OCBBF2008775, OCBBF2008790, OCBBF2008814, OCBBF2008822, OCBBF2009095, OCBBF2009115, OCBBF2009301, OCBBF2009352, OCBBF2009391, OCBBF2009424, OCBBF2009536, OCBBF2009571, OCBBF2009583, OCBBF2009603, OCBBF2009772, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010841, OCBBF2010843, OCBBF2010858, OCBBF2010863, OCBBF2010871, OCBBF2010931, OCBBF2010945, OCBBF2010978, OCBBF2010989, OCBBF2011021, OCBBF2011073, OCBBF2011137, OCBBF2011160, OCBBF2011177, OCBBF2011228, OCBBF2011232, OCBBF2011283, OCBBF2011311, OCBBF2011536,

OCBBF2011625, OCBBF2011669, OCBBF2011685, OCBBF2011706, OCBBF2011722, OCBBF2011759, OCBBF2011767, OCBBF2011855, OCBBF2011872, OCBBF2011887, OCBBF2011897, OCBBF2011914, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012553, OCBBF2012678, OCBBF2012704, OCBBF2012714, OCBBF2012755, OCBBF2012812, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013481, OCBBF2013507, OCBBF2013721, OCBBF2013789, OCBBF2013843, OCBBF2013883,

OCBBF2013926, OCBBF2014052, OCBBF2014064, OCBBF2014089, OCBBF2014292, OCBBF2014322, OCBBF2014386, OCBBF2014541, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016841, OCBBF2016928, OCBBF2017035, OCBBF2017055, OCBBF2017069, OCBBF2017306, OCBBF2017325, OCBBF2017398, OCBBF2017458, OCBBF2017489,

OCBBF2017516, OCBBF2017665, OCBBF2017754, OCBBF2017791, OCBBF2017815, OCBBF2017882, OCBBF2017888, OCBBF2017893, OCBBF2017920, OCBBF2018012, OCBBF2018014, OCBBF2018084, OCBBF2018167, OCBBF2018206, OCBBF2018229, OCBBF2018234, OCBBF2018362, OCBBF2018380, OCBBF2018563, OCBBF2018581, OCBBF2018618, OCBBF2018663, OCBBF2018687, OCBBF2018707, OCBBF2018827, OCBBF2018828, OCBBF2018873, OCBBF2018934, OCBBF2018956, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021286, OCBBF2021323, OCBBF2021518, OCBBF2021788,

OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574,

OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035226, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296,

OCBBF3000323, OCBBF3000372, OCBBF3000380, OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004487, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000086, PANCR1000091, PANCR1000185, PEBLM1000024, PEBLM1000029, PEBLM1000034, PEBLM1000062, PEBLM1000066, PEBLM1000068, PEBLM1000071, PEBLM1000083, PEBLM1000144, PEBLM1000147, PEBLM1000174,

PEBLM1000180, PEBLM2000030, PEBLM2000112, PEBLM2000126, PEBLM2000147, PEBLM2000170, PEBLM2000180, PEBLM2000213, PEBLM2000222, PEBLM2000248, PEBLM2000267, PEBLM2000270, PEBLM2000308, PEBLM2000321, PEBLM2000326, PEBLM2000333, PEBLM2000338, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004452, PEBLM2004497, PEBLM2004666, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215,

PEBLM2006283, PEBLM2006298, PEBLM2006366, PEBLM2006709, PEBLM2006912, PEBLM2007112, PEBLM2007140, PEBLM2007188, PEBLM2007296, PEBLM2007437, PEBLM2007447, PEBLM2007598, PEBLM2007774, PEBLM2007832, PEBLM2007834, PEBLM2008106, PEBLM2008576, PEBLM2008605, PEBLM2008826, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919,

PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007068, PERIC2007439, PERIC2007914, PERIC2008008, PERIC2008385, PERIC2009001, PERIC2009086, PERIC2009329, PERIC2009566, PLACE5000001, PLACE5000013, PLACE5000037, PLACE5000058, PLACE5000066, PLACE5000067, PLACE5000068, PLACE5000070, PLACE5000080, PLACE5000105, PLACE5000113, PLACE5000115, PLACE5000116, PLACE5000129, PLACE5000139, PLACE5000153, PLACE5000159, PLACE5000170, PLACE5000171, PLACE5000172, PLACE5000178,

PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000145, PLACE6000191, PLACE6000205, PLACE6000231, PLACE6000263, PLACE6000296, PLACE6000314, PLACE6000325, PLACE6000348, PLACE6000371, PLACE6000379, PLACE6000414, PLACE6000424, PLACE6000426, PLACE6000429, PLACE6000450, PLACE6000463, PLACE6000523, PLACE6000550, PLACE6000552, PLACE6000555, PLACE6000619, PLACE6000953, PLACE6001064, PLACE6001118, PLACE6001153, PLACE6001185, PLACE6001281, PLACE6001294, PLACE6001443, PLACE6001536, PLACE6001557, PLACE6001712, PLACE6001740, PLACE6001823, PLACE6001886, PLACE6001923,

PLACE6001925, PLACE6001933, PLACE6002011, PLACE6002016, PLACE6002056, PLACE6002084, PLACE6002102, PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6002960, PLACE6003004, PLACE6003077, PLACE6003094, PLACE6003109, PLACE6003204, PLACE6003218, PLACE6003372, PLACE6003383, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004005, PLACE6004101, PLACE6004219, PLACE6004336, PLACE6004368, PLACE6004380, PLACE6004396, PLACE6004397, PLACE6004454, PLACE6004464,

PLACE6004491, PLACE6004578, PLACE6004663, PLACE6004687, PLACE6004738, PLACE6004931, PLACE6004966, PLACE6004993, PLACE6005007, PLACE6005029, PLACE6005108, PLACE6005231, PLACE6005234, PLACE6005283, PLACE6005294, PLACE6005328, PLACE6005351, PLACE6005423, PLACE6005482, PLACE6005535, PLACE6005546, PLACE6005559, PLACE6005579, PLACE6005691, PLACE6005786, PLACE6006042, PLACE6006077, PLACE6006137, PLACE6006158, PLACE6006186, PLACE6006217, PLACE6006222, PLACE6006266, PLACE6006287, PLACE6006394, PLACE6006418, PLACE6006474, PLACE6006549, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373,

PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008775, PLACE6008793, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010077, PLACE6010463, PLACE6010484, PLACE6010485, PLACE6010568, PLACE6010701, PLACE6010704, PLACE6010732, PLACE6010765, PLACE6010848, PLACE6010925, PLACE6010936, PLACE6010991,

PLACE6011041, PLACE6011057, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012908, PLACE6012936, PLACE6012942, PLACE6013222, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210,

PLACE6016254, PLACE6016383, PLACE6016942, PLACE6016984, PLACE6017002, PLACE6017431, PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6018938, PLACE6019195, PLACE6019385, PLACE6019542, PLACE6019600, PLACE6019674, PLACE6019676, PLACE6019701, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657,

PLACE7003684, PLACE7003985, PLACE7004103, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000097, PROST1000110, PROST1000120, PROST1000123, PROST1000136, PROST1000144, PROST1000145, PROST1000152, PROST1000167, PROST1000184, PROST1000199, PROST1000215, PROST1000217, PROST1000220, PROST1000226, PROST1000246, PROST1000262, PROST1000272, PROST1000298, PROST1000322, PROST1000334, PROST1000343,

PROST1000362, PROST1000377, PROST1000383, PROST1000412, PROST1000419, PROST1000451, PROST1000480, PROST1000485, PROST1000493, PROST1000512, PROST1000526, PROST1000527, PROST1000528, PROST1000536, PROST1000559, PROST1000564, PROST1000575, PROST2000036, PROST2000053, PROST2000067, PROST2000105, PROST2000143, PROST2000162, PROST2000165, PROST2000176, PROST2000203, PROST2000206, PROST2000212, PROST2000241, PROST2000267, PROST2000273, PROST2000274, PROST2000277, PROST2000284, PROST2000325, PROST2000337, PROST2000432, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002,

PROST2001116, PROST2001138, PROST2001180, PROST2001213, PROST2001283, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001823, PROST2001883, PROST2001899, PROST2001997, PROST2001998, PROST2002078, PROST2002101, PROST2002212, PROST2002227, PROST2002338, PROST2002425, PROST2002488, PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003232, PROST2003302, PROST2003303,

PROST2003324, PROST2003338, PROST2003340, PROST2003396, PROST2003428, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004095, PROST2004115, PROST2004142, PROST2004146, PROST2004161, PROST2004258, PROST2004270, PROST2004332, PROST2004416, PROST2004481, PROST2004526, PROST2004570, PROST2004727, PROST2004739, PROST2004742, PROST2004744, PROST2004817, PROST2005039, PROST2005067, PROST2005076, PROST2005093, PROST2005121, PROST2005131, PROST2005143, PROST2005228, PROST2005272, PROST2005285, PROST2005426, PROST2005466,

PROST2005604, PROST2005630, PROST2005683, PROST2005788, PROST2005793, PROST2005880, PROST2005886, PROST2005904, PROST2005919, PROST2005943, PROST2006008, PROST2006020, PROST2006030, PROST2006060, PROST2006196, PROST2006201, PROST2006260, PROST2006282, PROST2006343, PROST2006450, PROST2006491, PROST2006510, PROST2006536, PROST2006579, PROST2006688, PROST2006737, PROST2006766, PROST2006782, PROST2006933, PROST2006988, PROST2007122, PROST2007200, PROST2007237, PROST2007248, PROST2007289, PROST2007317, PROST2007328, PROST2007382, PROST2007389, PROST2007416, PROST2007528, PROST2007612, PROST2007818, PROST2007871, PROST2007950, PROST2007956, PROST2007974, PROST2007988, PROST2008079, PROST2008088,

PROST2008243, PROST2008245, PROST2008268, PROST2008271, PROST2008360, PROST2008447, PROST2008468, PROST2008472, PROST2008489, PROST2008516, PROST2008724, PROST2008770, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010579, PROST2010606, PROST2010782, PROST2010885, PROST2011012, PROST2011038, PROST2011105, PROST2011297, PROST2011410, PROST2011439,

PROST2011482, PROST2011577, PROST2011631, PROST2011660, PROST2012005, PROST2012007, PROST2012016, PROST2012088, PROST2012140, PROST2012157, PROST2012190, PROST2012249, PROST2012353, PROST2012400, PROST2012448, PROST2012504, PROST2012542, PROST2012550, PROST2012740, PROST2012745, PROST2012780, PROST2012888, PROST2012890, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015137, PROST2015198, PROST2015243, PROST2015246, PROST2015251, PROST2015287, PROST2015332, PROST2015457, PROST2015464,

PROST2015537, PROST2015545, PROST2015551, PROST2015636, PROST2015710, PROST2015756, PROST2015877, PROST2015900, PROST2015924, PROST2015932, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2016980, PROST2017098, PROST2017128, PROST2017203, PROST2017367, PROST2017413, PROST2017441, PROST2017529, PROST2017578, PROST2017612, PROST2017617, PROST2017692, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583,

PROST2018588, PROST2018607, PROST2018788, PROST2018902, PROST2018922, PROST2018977, PROST2019164, PROST2019253, PROST2019296, PROST2019398, PROST2019487, PROST2019781, PUAEN1000039, PUAEN1000057, PUAEN1000065, PUAEN1000081, PUAEN1000085, PUAEN1000087, PUAEN1000121, PUAEN1000147, PUAEN1000161, PUAEN1000164, PUAEN1000175, PUAEN1000239, PUAEN1000275, PUAEN1000276, PUAEN1000288, PUAEN2000080, PUAEN2000152, PUAEN2000175, PUAEN2000247, PUAEN2000312, PUAEN2000394, PUAEN2000497, PUAEN2000535, PUAEN2000594, PUAEN2000684, PUAEN2000942, PUAEN2001260, PUAEN2001345, PUAEN2001526, PUAEN2001586, PUAEN2001882, PUAEN2002473, PUAEN2002489, PUAEN2002552, PUAEN2002568, PUAEN2002616, PUAEN2002758, PUAEN2003018,

PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029, PUAEN2006328, PUAEN2006335, PUAEN2006578, PUAEN2006639, PUAEN2006761, PUAEN2007044, PUAEN2007350, PUAEN2007785, PUAEN2007898, PUAEN2008123, PUAEN2008228, PUAEN2008314, PUAEN2008515, PUAEN2008815, PUAEN2008939, PUAEN2008980, PUAEN2008985, PUAEN2009174, PUAEN2009348, PUAEN2009534, PUAEN2009655, PUAEN2009795, PUAEN2009852, PUAEN2010346, PUAEN2010824, RECTM1000051, RECTM1000141, RECTM2000220, RECTM2000349, RECTM2000433, RECTM2000510, RECTM2001307, RECTM2001347,

RECTM2001519, RECTM2001666, RECTM2001691, RECTM2001749, RECTM2002172, RECTM2002602, RECTM2002632, SALGL1000005, SALGL1000028, SALGL1000047, SALGL1000065, SALGL1000107, SALGL1000171, SKMUS1000014, SKMUS1000022, SKMUS1000030, SKMUS1000064, SKMUS1000067, SKMUS1000084, SKMUS1000102, SKMUS1000104, SKMUS1000110, SKMUS1000118, SKMUS1000124, SKMUS1000129, SKMUS1000138, SKMUS1000176, SKMUS1000177, SKMUS2000020, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000361, SKMUS2000365, SKMUS2000380, SKMUS2000390, SKMUS2000416, SKMUS2000458, SKMUS2000467, SKMUS2000468,

SKMUS2000484, SKMUS2000515, SKMUS2000679, SKMUS2000690, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000873, SKMUS2000894, SKMUS2000898, SKMUS2000902, SKMUS2000931, SKMUS2000933, SKMUS2000945, SKMUS2000954, SKMUS2000961, SKMUS2000973, SKMUS2000980, SKMUS2001008, SKMUS2001014, SKMUS2001129, SKMUS2001147, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001454, SKMUS2001492, SKMUS2001501, SKMUS2001543, SKMUS2001580, SKMUS2001585,

SKMUS2001608, SKMUS2001631, SKMUS2001634, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474,

SKMUS2008585, SKMUS2008607, SKMUS2009190, SKMUS2009232, SKMUS2009479, SKMUS2009557, SKNMC1000110, SKNMC1000123, SKNMC1000124, SKNMC1000137, SKNMC1000159, SKNMC1000168, SKNMC1000229, SKNMC1000251, SKNMC1000264, SKNMC2000065, SKNMC2000097, SKNMC2000224, SKNMC2000256, SKNMC2000305, SKNMC2000356, SKNMC2000374, SKNMC2000583, SKNMC2000593, SKNMC2000612, SKNMC2000622, SKNMC2000649, SKNMC2000698, SKNMC2000877, SKNMC2000966, SKNMC2001113, SKNMC2001324, SKNMC2001503, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504,

SKNMC2007961, SKNMC2009450, SKNSH1000002, SKNSH1000086, SKNSH1000101, SKNSH1000174, SKNSH1000301, SKNSH1000308, SKNSH1000416, SKNSH2000051, SKNSH2000101, SKNSH2000151, SKNSH2000163, SKNSH2000250, SKNSH2000319, SKNSH2000347, SKNSH2000482, SKNSH2000516, SKNSH2000550, SKNSH2000716, SKNSH2000819, SKNSH2000971, SKNSH2001222, SKNSH2001875, SKNSH2001931, SKNSH2002054, SKNSH2002325, SKNSH2002768, SKNSH2002866, SKNSH2003064, SKNSH2003174, SKNSH2003422, SKNSH2003466, SKNSH2003483, SKNSH2003528, SKNSH2003633, SKNSH2004039, SKNSH2005061, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149,

SKNSH2007429, SKNSH2007739, SKNSH2008043, SKNSH2008940, SKNSH2008969, SKNSH2009197, SKNSH2009357, SKNSH2009435, SKNSH2009991, SKNSH2010015, SMINT1000012, SMINT1000016, SMINT1000039, SMINT1000042, SMINT1000048, SMINT1000051, SMINT1000054, SMINT1000057, SMINT1000071, SMINT1000075, SMINT1000078, SMINT1000091, SMINT1000100, SMINT1000103, SMINT1000117, SMINT1000118, SMINT1000137, SMINT1000192, SMINT2000007, SMINT2000018, SMINT2000032, SMINT2000040, SMINT2000060, SMINT2000073, SMINT2000084, SMINT2000145, SMINT2000159, SMINT2000176, SMINT2000185, SMINT2000227, SMINT2000229, SMINT2000232, SMINT2000239, SMINT2000267, SMINT2000277, SMINT2000396, SMINT2000400, SMINT2000441, SMINT2000454, SMINT2000468,

SMINT2000518, SMINT2000529, SMINT2000530, SMINT2000537, SMINT2000538, SMINT2000541, SMINT2000558, SMINT2000562, SMINT2000568, SMINT2000602, SMINT2000609, SMINT2000629, SMINT2000659, SMINT2000694, SMINT2000747, SMINT2000811, SMINT2000824, SMINT2000984, SMINT2001114, SMINT2001158, SMINT2001183, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001950, SMINT2002126, SMINT2002159, SMINT2002202, SMINT2002210, SMINT2002224, SMINT2002281, SMINT2002311, SMINT2002314, SMINT2002328, SMINT2002414, SMINT2002457, SMINT2002620, SMINT2002689, SMINT2002743, SMINT2002778, SMINT2002880,

SMINT2002882, SMINT2002899, SMINT2002974, SMINT2002976, SMINT2003003, SMINT2003074, SMINT2003128, SMINT2003150, SMINT2003169, SMINT2003317, SMINT2003340, SMINT2003386, SMINT2003423, SMINT2003551, SMINT2003569, SMINT2003641, SMINT2003644, SMINT2003866, SMINT2004037, SMINT2004047, SMINT2004086, SMINT2004144, SMINT2004280, SMINT2004299, SMINT2004339, SMINT2004350, SMINT2004414, SMINT2004473, SMINT2004547, SMINT2004589, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005368, SMINT2005387, SMINT2005402, SMINT2005405, SMINT2005621, SMINT2005623, SMINT2005624, SMINT2005800,

SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2008921,

SMINT2009119, SMINT2009121, SMINT2009212, SMINT2009216, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010901, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958,

SMINT2012040, SMINT2012122, SMINT2012179, SMINT2012195, SMINT2012285, SMINT2012291, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014489, SMINT2014721, SMINT2015006, SMINT2015294, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432,

SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000091, SPLEN1000106, SPLEN1000134, SPLEN1000139, SPLEN1000143, SPLEN1000144, SPLEN1000145, SPLEN1000156, SPLEN1000163, SPLEN1000165, SPLEN1000166, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000210, SPLEN2000222, SPLEN2000242, SPLEN2000243,

SPLEN2000247, SPLEN2000255, SPLEN2000258, SPLEN2000267, SPLEN2000270, SPLEN2000307, SPLEN2000310, SPLEN2000341, SPLEN2000348, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000516, SPLEN2000537, SPLEN2000541, SPLEN2000607, SPLEN2000630, SPLEN2000695, SPLEN2000839, SPLEN2001135, SPLEN2001157, SPLEN2001176, SPLEN2001227, SPLEN2001245, SPLEN2001278, SPLEN2001354, SPLEN2001367, SPLEN2001383, SPLEN2001476, SPLEN2001510, SPLEN2001599, SPLEN2001626, SPLEN2001650, SPLEN2001689, SPLEN2001710, SPLEN2001761, SPLEN2001781, SPLEN2001881, SPLEN2001912, SPLEN2001945, SPLEN2002007,

SPLEN2002053, SPLEN2002133, SPLEN2002147, SPLEN2002166, SPLEN2002175, SPLEN2002223, SPLEN2002272, SPLEN2002314, SPLEN2002335, SPLEN2002343, SPLEN2002354, SPLEN2002385, SPLEN2002419, SPLEN2002451, SPLEN2002462, SPLEN2002463, SPLEN2002477, SPLEN2002493, SPLEN2002662, SPLEN2002695, SPLEN2002707, SPLEN2002744, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004220, SPLEN2004343, SPLEN2004346, SPLEN2004368, SPLEN2004555, SPLEN2004611, SPLEN2004662, SPLEN2004773, SPLEN2004844, SPLEN2004880, SPLEN2005009, SPLEN2005227,

SPLEN2005272, SPLEN2005416, SPLEN2005429, SPLEN2005450, SPLEN2005474, SPLEN2005560, SPLEN2005727, SPLEN2005783, SPLEN2005790, SPLEN2005818, SPLEN2005838, SPLEN2005869, SPLEN2005927, SPLEN2005939, SPLEN2006133, SPLEN2006143, SPLEN2006232, SPLEN2006268, SPLEN2006283, SPLEN2006305, SPLEN2006325, SPLEN2006374, SPLEN2006389, SPLEN2006425, SPLEN2006671, SPLEN2006701, SPLEN2006785, SPLEN2006875, SPLEN2007182, SPLEN2007314, SPLEN2007326, SPLEN2007350, SPLEN2007388, SPLEN2007498, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008496,

SPLEN2008591, SPLEN2008786, SPLEN2009081, SPLEN2009088, SPLEN2009163, SPLEN2009315, SPLEN2009340, SPLEN2009512, SPLEN2009513, SPLEN2009541, SPLEN2009548, SPLEN2009555, SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010195, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252,

SPLEN2011419, SPLEN2011422, SPLEN2011672, SPLEN2011737, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012523, SPLEN2012524, SPLEN2012525, SPLEN2012606, SPLEN2012611, SPLEN2012619, SPLEN2012624, SPLEN2012725, SPLEN2012743, SPLEN2012756, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014080,

SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014911, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015310, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045,

SPLEN2016053, SPLEN2016069, SPLEN2016098, SPLEN2016135, SPLEN2016139, SPLEN2016241, SPLEN2016250, SPLEN2016268, SPLEN2016304, SPLEN2016356, SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016773, SPLEN2016781, SPLEN2016840, SPLEN2016863, SPLEN2016922, SPLEN2016932, SPLEN2016972, SPLEN2016973, SPLEN2017043, SPLEN2017104, SPLEN2017121, SPLEN2017147, SPLEN2017187, SPLEN2017189, SPLEN2017212, SPLEN2017318, SPLEN2017351, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017999, SPLEN2018098,

SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019169, SPLEN2019257, SPLEN2019311, SPLEN2019323, SPLEN2019349, SPLEN2019375, SPLEN2019379, SPLEN2019405, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223,

SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024273, SPLEN2024383, SPLEN2024489, SPLEN2024530, SPLEN2024571, SPLEN2024577, SPLEN2024617, SPLEN2024737, SPLEN2024922,

SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027113, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424,

SPLEN2031547, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678,

SPLEN2037722, SPLEN2037891, SPLEN2038055, SPLEN2038180, SPLEN2038345, SPLEN2038407, SPLEN2038473, SPLEN2038834, SPLEN2039311, SPLEN2039379, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042535, SPLEN2042598, SPLEN2042714, SPLEN2042920, STOMA1000013, STOMA1000047, STOMA1000052, STOMA1000074, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000121, STOMA2000148, STOMA2000168,

STOMA2000183, STOMA2000188, STOMA2000257, STOMA2000279, STOMA2000289, STOMA2000383, STOMA2000386, STOMA2000395, STOMA2000396, STOMA2000478, STOMA2000482, STOMA2000529, STOMA2000539, STOMA2000567, STOMA2000640, STOMA2000678, STOMA2000686, STOMA2000888, STOMA2001025, STOMA2001389, STOMA2002052, STOMA2002141, STOMA2002166, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213,

STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049, SYNOV1000118, SYNOV1000124, SYNOV1000128, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001033, SYNOV2001035, SYNOV2001088, SYNOV2001111,

SYNOV2001144, SYNOV2001212, SYNOV2001251, SYNOV2001300, SYNOV2001356, SYNOV2001374, SYNOV2001390, SYNOV2001451, SYNOV2001457, SYNOV2001468, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2009172, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001153, SYNOV4001224,

SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007671, SYNOV4007711, SYNOV4008245, SYNOV4008336, SYNOV4009129, SYNOV4009139, SYNOV4009298, SYNOV4009575, T1ESE2000116, T1ESE2000609, T1ESE2000904, T1ESE2002665, TBAES2000004, TBAES2000059, TBAES2000116, TBAES2000255, TBAES2000315, TBAES2000352, TBAES2000932, TBAES2001171,

TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TBAES2009387, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312,

TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002273, TESOP2002451, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865,

TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000023, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000163, TESTI1000168, TESTI1000169, TESTI1000179,

TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000172, TESTI2000177, TESTI2000179, TESTI2000184, TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000598, TESTI2000600, TESTI2000616, TESTI2000627, TESTI2000644, TESTI2000671,

TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082, TESTI2001099, TESTI2001120, TESTI2001134, TESTI2001141, TESTI2001153, TESTI2001180, TESTI2001208, TESTI2001229, TESTI2001236, TESTI2001237, TESTI2001269, TESTI2001306, TESTI2001313, TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593,

TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001829, TESTI2001852, TESTI2001869, TESTI2001879, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002365,

TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002707, TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003020, TESTI2003031, TESTI2003037, TESTI2003044, TESTI2003059, TESTI2003061, TESTI2003074, TESTI2003089, TESTI2003104, TESTI2003109, TESTI2003117, TESTI2003130, TESTI2003131,

TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003413, TESTI2003418, TESTI2003419, TESTI2003432, TESTI2003454, TESTI2003475, TESTI2003476, TESTI2003498, TESTI2003512, TESTI2003533, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727,

TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004080, TESTI2004085, TESTI2004089, TESTI2004111, TESTI2004119, TESTI2004122, TESTI2004153, TESTI2004163, TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675,

TESTI2004687, TESTI2004689, TESTI2004700, TESTI2004706, TESTI2004712, TESTI2004734, TESTI2004737, TESTI2004783, TESTI2004791, TESTI2004793, TESTI2004860, TESTI2004906, TESTI2004920, TESTI2004929, TESTI2004936, TESTI2004941, TESTI2004971, TESTI2004982, TESTI2004993, TESTI2004994, TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005408, TESTI2005415, TESTI2005470, TESTI2005492, TESTI2005564, TESTI2005568,

TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005743, TESTI2005759, TESTI2005767, TESTI2005775, TESTI2005784, TESTI2005788, TESTI2005827, TESTI2005835, TESTI2005860, TESTI2005892, TESTI2005908, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006051, TESTI2006067, TESTI2006083, TESTI2006097, TESTI2006109,

TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006453, TESTI2006457, TESTI2006460, TESTI2006465, TESTI2006483, TESTI2006499, TESTI2006523, TESTI2006543, TESTI2006565, TESTI2006572, TESTI2006615, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006748, TESTI2006774, TESTI2006853, TESTI2006866, TESTI2006872, TESTI2006877,

TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007311, TESTI2007320, TESTI2007346, TESTI2007358, TESTI2007402, TESTI2007405, TESTI2007407, TESTI2007422, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972,

TESTI2007973, TESTI2007998, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008240, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960,

TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009477, TESTI2009497, TESTI2009502, TESTI2009511, TESTI2009520, TESTI2009544, TESTI2009545, TESTI2009551, TESTI2009577, TESTI2009585, TESTI2009588, TESTI2009683, TESTI2009727, TESTI2009739, TESTI2009785, TESTI2009812, TESTI2009835, TESTI2009853, TESTI2009935, TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591,

TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012050, TESTI2012090, TESTI2012095, TESTI2012104, TESTI2012152, TESTI2012155, TESTI2012171, TESTI2012207, TESTI2012231, TESTI2012244, TESTI2012289, TESTI2012308, TESTI2012356,

TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013257, TESTI2013264, TESTI2013268, TESTI2013295, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889,

TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988, TESTI2015042, TESTI2015092, TESTI2015105, TESTI2015180, TESTI2015246, TESTI2015249, TESTI2015327, TESTI2015331, TESTI2015335, TESTI2015350, TESTI2015375, TESTI2015590, TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033,

TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816,

TESTI2017832, TESTI2017923, TESTI2017932, TESTI2017951, TESTI2018014, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018462, TESTI2018475, TESTI2018482, TESTI2018521, TESTI2018565, TESTI2018581, TESTI2018611, TESTI2018687, TESTI2018697, TESTI2018838, TESTI2018867, TESTI2018941, TESTI2018957, TESTI2018974, TESTI2018975, TESTI2018997, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648,

TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021124, TESTI2021138,

TESTI2021297, TESTI2021315, TESTI2021358, TESTI2021425, TESTI2021447, TESTI2021463, TESTI2021531, TESTI2021599, TESTI2021637, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414,

TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024885, TESTI2024936, TESTI2024978, TESTI2024999, TESTI2025006, TESTI2025022, TESTI2025063, TESTI2025117, TESTI2025144, TESTI2025161, TESTI2025174, TESTI2025229, TESTI2025269, TESTI2025365,

TESTI2025403, TESTI2025409, TESTI2025422, TESTI2025448, TESTI2025454, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026534, TESTI2026537, TESTI2026589, TESTI2026597, TESTI2026605, TESTI2026647, TESTI2026674, TESTI2026794, TESTI2026824, TESTI2026854, TESTI2026925,

TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776,

TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643,

TESTI2032681, TESTI2032761, TESTI2032768, TESTI2032774, TESTI2032828, TESTI2033031, TESTI2033242, TESTI2033254, TESTI2033300, TESTI2033395, TESTI2033441, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309,

TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065,

TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362,

TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796, TESTI2044833,

TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046456, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109,

TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723,

TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665,

TESTI4001679, TESTI4001795, TESTI4001923, TESTI4001925, TESTI4001984, TESTI4002003, TESTI4002072, TESTI4002141, TESTI4002195, TESTI4002202, TESTI4002290, TESTI4002319, TESTI4002491, TESTI4002520, TESTI4002552, TESTI4002647, TESTI4002703, TESTI4002754, TESTI4002799, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133,

TESTI4005158, TESTI4005317, TESTI4005322, TESTI4005399, TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799,

TESTI4007810, TESTI4007816, TESTI4007965, TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475,

TESTI4010544, TESTI4010713, TESTI4010721, TESTI4010789, TESTI4010817, TESTI4010831, TESTI4010851, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013474, TESTI4013602, TESTI4013667, TESTI4013675, TESTI4013685, TESTI4013735,

TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865,

TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920,

TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390,

TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335,

TESTI4031745, TESTI4031818, TESTI4032090, TESTI4032112, TESTI4032128, TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037156, TESTI4037188, TESTI4037228, TESTI4037244, TESTI4037727,

TESTI4037949, TESTI4038047, TESTI4038099, TESTI4038156, TESTI4038223, TESTI4038258, TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711,

TESTI4042846, TESTI4043067, TESTI4043129, TESTI4043140, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903,

TESTI4046962, TESTI4047069, TESTI4047119, TESTI4047305, TESTI4047328, TESTI4047437, TESTI4047569, TESTI4047746, TESTI4047808, THYMU1000002, THYMU1000010, THYMU1000017, THYMU1000025, THYMU1000029, THYMU1000032, THYMU1000033, THYMU1000041, THYMU1000060, THYMU1000074, THYMU1000083, THYMU1000098, THYMU1000103, THYMU1000105, THYMU1000109, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000329, THYMU1000331, THYMU1000336, THYMU1000366, THYMU1000374, THYMU1000382, THYMU1000393, THYMU1000399, THYMU1000426, THYMU1000428, THYMU1000440, THYMU1000459,

THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000382, THYMU2000418, THYMU2000436, THYMU2000440, THYMU2000489, THYMU2000492, THYMU2000519, THYMU2000570, THYMU2000602, THYMU2000660, THYMU2000671, THYMU2000672, THYMU2000684, THYMU2000702, THYMU2000767, THYMU2000775, THYMU2000800, THYMU2000879,

THYMU2000932, THYMU2000946, THYMU2000950, THYMU2000971, THYMU2000982, THYMU2000990, THYMU2001007, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001839, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002162, THYMU2002177, THYMU2002203, THYMU2002229, THYMU2002242,

THYMU2002338, THYMU2002356, THYMU2002376, THYMU2002384, THYMU2002450, THYMU2002490, THYMU2002548, THYMU2002583, THYMU2002695, THYMU2002745, THYMU2002756, THYMU2002789, THYMU2002792, THYMU2002815, THYMU2002841, THYMU2002852, THYMU2002910, THYMU2002950, THYMU2002983, THYMU2003046, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003446, THYMU2003470, THYMU2003479, THYMU2003499, THYMU2003542, THYMU2003632, THYMU2003650, THYMU2003700, THYMU2003760, THYMU2003803, THYMU2003855, THYMU2003907, THYMU2003932, THYMU2004003, THYMU2004008,

THYMU2004042, THYMU2004108, THYMU2004111, THYMU2004123, THYMU2004139, THYMU2004152, THYMU2004169, THYMU2004284, THYMU2004320, THYMU2004336, THYMU2004344, THYMU2004356, THYMU2004410, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005546, THYMU2005574,

THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006303, THYMU2006324, THYMU2006350, THYMU2006357, THYMU2006365, THYMU2006420, THYMU2006468, THYMU2006505, THYMU2006610, THYMU2006666, THYMU2006712, THYMU2006813, THYMU2006913, THYMU2006946, THYMU2006965, THYMU2007025, THYMU2007036, THYMU2007065, THYMU2007095, THYMU2007112, THYMU2007146, THYMU2007179, THYMU2007258, THYMU2007307, THYMU2007308, THYMU2007339, THYMU2007350, THYMU2007384, THYMU2007415, THYMU2007467, THYMU2007493, THYMU2007521, THYMU2007528,

THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101,

THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010448, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011482, THYMU2011538,

THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012024, THYMU2012073, THYMU2012104, THYMU2012113, THYMU2012273, THYMU2012401, THYMU2012492, THYMU2012507, THYMU2012565, THYMU2012574, THYMU2012625, THYMU2012631, THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013136, THYMU2013170, THYMU2013364, THYMU2013386, THYMU2013426, THYMU2013578, THYMU2013648, THYMU2013705, THYMU2013757,

THYMU2013779, THYMU2013863, THYMU2013916, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015762, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017158,

THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020491, THYMU2020499, THYMU2020560, THYMU2020667, THYMU2020830,

THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734,

THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696,

THYMU2032715, THYMU2032732, THYMU2032976, THYMU20.33053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2035735, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234,

THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504,

THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485,

THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013386, THYMU3013470, THYMU3013785, THYMU3013897, THYMU3014038, THYMU3014173, THYMU3014372, THYMU3014555, THYMU3014620, THYMU3014701,

THYMU3015042, THYMU3015457, THYMU3015571, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642,

THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032867, THYMU3033402, THYMU3033626, THYMU3033630, THYMU3033649, THYMU3033754, THYMU3033759, THYMU3034078, THYMU3034099, THYMU3034453, THYMU3034616, THYMU3034671, THYMU3034853, THYMU3034867, THYMU3034983,

THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758,

THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000066, TKIDN1000164, TKIDN1000171, TKIDN1000192, TKIDN2000127, TKIDN2000240, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2003044, TKIDN2003059,

TKIDN2003062, TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005487, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025,

TKIDN2015071, TKIDN2015161, TKIDN2015263, TKIDN2015285, TKIDN2015423, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001616, TLIVE2001684, TLIVE2001828, TLIVE2001927, TLIVE2002046, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192,

TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000030, TRACH1000038, TRACH1000057, TRACH1000063, TRACH1000074, TRACH1000100, TRACH1000106, TRACH1000125, TRACH1000140, TRACH1000144, TRACH1000181, TRACH1000193, TRACH1000205, TRACH1000212, TRACH1000240, TRACH2000002, TRACH2000015, TRACH2000024, TRACH2000032, TRACH2000045, TRACH2000047,

TRACH2000081, TRACH2000083, TRACH2000148, TRACH2000185, TRACH2000235, TRACH2000237, TRACH2000243, TRACH2000248, TRACH2000250, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000420, TRACH2000461, TRACH2000472, TRACH2000496, TRACH2000497, TRACH2000502, TRACH2000522, TRACH2000559, TRACH2000660, TRACH2000665, TRACH2000675, TRACH2000677, TRACH2000702, TRACH2000703, TRACH2000767, TRACH2000775, TRACH2000807, TRACH2000862, TRACH2000873, TRACH2000894, TRACH2000898, TRACH2000920, TRACH2000923, TRACH2000926, TRACH2000944, TRACH2000959, TRACH2000972, TRACH2000981, TRACH2000986, TRACH2001021,

TRACH2001037, TRACH2001060, TRACH2001085, TRACH2001101, TRACH2001121, TRACH2001147, TRACH2001154, TRACH2001188, TRACH2001192, TRACH2001249, TRACH2001275, TRACH2001289, TRACH2001314, TRACH2001335, TRACH2001339, TRACH2001341, TRACH2001347, TRACH2001395, TRACH2001400, TRACH2001403, TRACH2001416, TRACH2001428, TRACH2001432, TRACH2001443, TRACH2001463, TRACH2001474, TRACH2001478, TRACH2001525, TRACH2001532, TRACH2001549, TRACH2001592, TRACH2001596, TRACH2001607, TRACH2001612, TRACH2001621, TRACH2001646, TRACH2001651, TRACH2001684, TRACH2001898, TRACH2001933, TRACH2001968, TRACH2001996, TRACH2002054, TRACH2002092, TRACH2002100, TRACH2002138, TRACH2002333, TRACH2002537, TRACH2002664, TRACH2002784,

TRACH2002803, TRACH2002840, TRACH2002847, TRACH2002853, TRACH2002862, TRACH2002954, TRACH2002960, TRACH2002988, TRACH2003070, TRACH2003272, TRACH2003323, TRACH2003347, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005800,

TRACH2005811, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007399, TRACH2007483, TRACH2007542, TRACH2007577, TRACH2007674, TRACH2007676, TRACH2007688, TRACH2007733, TRACH2007816, TRACH2007894, TRACH2007969, TRACH2007988, TRACH2008112, TRACH2008113, TRACH2008127, TRACH2008130, TRACH2008278, TRACH2008300, TRACH2008472, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268,

TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011113, TRACH2011290, TRACH2011302, TRACH2011500, TRACH2011574, TRACH2011894, TRACH2011956, TRACH2012000, TRACH2012138, TRACH2012242, TRACH2012298, TRACH2012370, TRACH2012376, TRACH2012387, TRACH2012497, TRACH2012536, TRACH2012562,

TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014077, TRACH2014082, TRACH2014124, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015823,

TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084, TRACH2018262, TRACH2018273, TRACH2018278, TRACH2018317, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080,

TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084,

TRACH2025155, TRACH2025224, TRACH2025344, TRACH2025379, TRACH2025507, TRACH2025535, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002192, TRACH3002245, TRACH3002293, TRACH3002533, TRACH3002561, TRACH3002567, TRACH3002605, TRACH3002650, TRACH3002752, TRACH3002866, TRACH3002871, TRACH3002876,

TRACH3002890, TRACH3003009, TRACH3003037, TRACH3003357, TRACH3003379, TRACH3003547, TRACH3003586, TRACH3003683, TRACH3003780, TRACH3003805, TRACH3003872, TRACH3004068, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005699, TRACH3005702, TRACH3005808, TRACH3005852, TRACH3005896, TRACH3006038, TRACH3006149, TRACH3006228, TRACH3006264,

TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007625, TRACH3007627, TRACH3007689, TRACH3007766, TRACH3007866, TRACH3007995, TRACH3008042, TRACH3008061, TRACH3008093, TRACH3008201, TRACH3008508, TRACH3008535, TRACH3008629, TRACH3008632, TRACH3008688, TRACH3008697, TRACH3008713, TRACH3008887, TRACH3008902, TRACH3008990, TRACH3009001, TRACH3009008, TRACH3009059, TRACH3009061, TRACH3009148, TRACH3009455, TRACH3009701, TRACH3009715,

TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136,

TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930,

TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681,

TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145,

TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036997, TRACH3037063, TRACH3037067, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274,

TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN1000186, UMVEN2000046, UMVEN2000069, UMVEN2000121, UMVEN2000152, UMVEN2000354, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131,

UTERU1000138, UTERU1000148, UTERU1000160, UTERU1000183, UTERU1000187, UTERU1000192, UTERU1000249, UTERU1000337, UTERU1000339, UTERU1000384, UTERU2000023, UTERU2000047, UTERU2000074, UTERU2000099, UTERU2000154, UTERU2000197, UTERU2000218, UTERU2000238, UTERU2000243, UTERU2000260, UTERU2000263, UTERU2000300, UTERU2000329, UTERU2000338, UTERU2000349, UTERU2000377, UTERU2000393, UTERU2000418, UTERU2000424, UTERU2000465, UTERU2000485, UTERU2000517, UTERU2000524, UTERU2000537, UTERU2000539, UTERU2000541, UTERU2000542, UTERU2000546, UTERU2000550, UTERU2000569, UTERU2000607, UTERU2000629, UTERU2000649, UTERU2000663, UTERU2000696, UTERU2000794, UTERU2000830, UTERU2000844, UTERU2000922, UTERU2000925,

UTERU2001024, UTERU2001110, UTERU2001176, UTERU2001281, UTERU2001389, UTERU2001409, UTERU2001412, UTERU2001504, UTERU2001607, UTERU2001658, UTERU2001747, UTERU2001876, UTERU2002001, UTERU2002011, UTERU2002176, UTERU2002198, UTERU2002294, UTERU2002332, UTERU2002410, UTERU2002473, UTERU2002547, UTERU2002662, UTERU2002693, UTERU2002733, UTERU2002736, UTERU2002737, UTERU2002826, UTERU2002841, UTERU2002964, UTERU2002993, UTERU2003035, UTERU2003057, UTERU2003126, UTERU2003135, UTERU2003321, UTERU2003399, UTERU2003411, UTERU2003456, UTERU2003577, UTERU2003704, UTERU2003926, UTERU2003973, UTERU2004015, UTERU2004037, UTERU2004039, UTERU2004061, UTERU2004073, UTERU2004163, UTERU2004197, UTERU2004299,

UTERU2004461, UTERU2004520, UTERU2004564, UTERU2004688, UTERU2004698, UTERU2004807, UTERU2004861, UTERU2004929, UTERU2005004, UTERU2005050, UTERU2005069, UTERU2005074, UTERU2005179, UTERU2005292, UTERU2005346, UTERU2005354, UTERU2005446, UTERU2005449, UTERU2005450, UTERU2005533, UTERU2005601, UTERU2005621, UTERU2005664, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547, UTERU2006568, UTERU2006593, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499,

UTERU2007639, UTERU2007724, UTERU2007924, UTERU2007942, UTERU2008018, UTERU2008019, UTERU2008040, UTERU2008077, UTERU2008130, UTERU2008302, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008938, UTERU2008939, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009510, UTERU2009538, UTERU2009540, UTERU2009776, UTERU2009904, UTERU2009951, UTERU2009972, UTERU2010115, UTERU2010124, UTERU2010164, UTERU2010226, UTERU2010231, UTERU2010304, UTERU2010320, UTERU2010431,

UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011806, UTERU2011811, UTERU2011897, UTERU2011906, UTERU2011962, UTERU2011968, UTERU2012031, UTERU2012101, UTERU2012114, UTERU2012230, UTERU2012252, UTERU2012286, UTERU2012333, UTERU2012407, UTERU2012526, UTERU2012581, UTERU2012610, UTERU2012615, UTERU2012688, UTERU2012703, UTERU2012715, UTERU2012741, UTERU2012767, UTERU2012786, UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502,

UTERU2013586, UTERU2013926, UTERU2013976, UTERU2014001, UTERU2014024, UTERU2014167, UTERU2014223, UTERU2014398, UTERU2014464, UTERU2014548, UTERU2014601, UTERU2014631, UTERU2014668, UTERU2014678, UTERU2014728, UTERU2014898, UTERU2014998, UTERU2015062, UTERU2015087, UTERU2015190, UTERU2015198, UTERU2015202, UTERU2015405, UTERU2015640, UTERU2015653, UTERU2015830, UTERU2015880, UTERU2016147, UTERU2016157, UTERU2016426, UTERU2016464, UTERU2016669, UTERU2016757, UTERU2016761, UTERU2016799, UTERU2016822, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810,

UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018955, UTERU2019005, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045,

UTERU2023175, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703,

UTERU2031834, UTERU2031851, UTERU2032075, UTERU2032220, UTERU2032279, UTERU2032726, UTERU2033172, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670,

UTERU3000727, UTERU3000738, UTERU3000828, UTERU3000844, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049,

UTERU3005159, UTERU3005205, UTERU3005230, UTERU3005264, UTERU3005422, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092,

UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the NT2NE, NT2R1, NT2RM, NT2RP, NTISM, NTONG, and OCBBF libraries were analyzed for expression frequency. The analysis results are described below. Each is a relative value, where the expression level over all tissues is taken as 100. The larger this value, the greater the level of expression.

TABLE 35

| Clone Name | NT2NE | NT2RI | NT2RM | NT2RP | NTISM | NTONG | OCBBF |
|---|---|---|---|---|---|---|---|
| 3NB691000116 | 0 | 0 | 0 | 0 | 0 | 0 | 2.020 |
| 3NB691000129 | 0 | 4.588 | 0 | 6.085 | 0 | 0 | 0 |
| 3NB691000173 | 5.723 | 0 | 0 | 0 | 0 | 0 | 0 |
| 3NB692000281 | 0 | 0.400 | 0 | 0.531 | 0 | 0 | 0 |
| 3NB692000374 | 0 | 0 | 0 | 0 | 0 | 0 | 2.694 |
| 3NB692000545 | 0 | 5.127 | 0 | 13.599 | 0 | 0 | 0 |
| 3NB692000912 | 5.108 | 0.854 | 0 | 1.132 | 0 | 3.463 | 0 |
| 3NB692001002 | 0 | 0 | 0 | 0 | 0 | 0 | 2.430 |
| 3NB692001022 | 0 | 0 | 0 | 0 | 0 | 0 | 3.091 |
| 3NB692001288 | 0 | 0 | 0 | 0 | 0 | 0 | 1.861 |
| 3NB692001334 | 1.946 | 0 | 0 | 0.647 | 0 | 1.978 | 1.667 |
| 3NB692001442 | 4.378 | 0 | 0 | 0 | 0 | 0 | 0 |
| 3NB692001496 | 27.896 | 0 | 0 | 0 | 0 | 0 | 0 |
| 3NB692001528 | 0 | 0 | 0 | 0 | 0 | 0 | 2.922 |
| 3NB692001719 | 0 | 0 | 0 | 0 | 0 | 0 | 8.556 |
| ADIPS2000425 | 0 | 0 | 0 | 0 | 0 | 0 | 0.964 |
| ADRGL1000002 | 0 | 0 | 0 | 0 | 0 | 0 | 53.185 |
| ADRGL1000065 | 0 | 0 | 0 | 0 | 0 | 0 | 7.006 |
| ADRGL1000144 | 0 | 0 | 0 | 16.110 | 0 | 0 | 4.152 |
| ADRGL2000006 | 3.767 | 1.889 | 0 | 0 | 0 | 0 | 12.914 |
| ADRGL2000042 | 9.204 | 0 | 0 | 0 | 0 | 0 | 0 |
| ADRGL2000097 | 0 | 0 | 0 | 5.358 | 0 | 0 | 0 |
| ADRGL2002477 | 0 | 0 | 0 | 0 | 0 | 0 | 4.227 |
| ADRGL2002679 | 0 | 0 | 0 | 0 | 0 | 0 | 15.458 |
| ADRGL2009273 | 0 | 0 | 0 | 0 | 0 | 0 | 3.451 |
| ADRGL2012179 | 0 | 0 | 0 | 0 | 0 | 0 | 17.655 |
| ASTRO1000165 | 0 | 8.055 | 0 | 0 | 0 | 0 | 11.014 |
| ASTRO2000046 | 0 | 10.381 | 0 | 4.917 | 0 | 0 | 1.014 |
| ASTRO2000381 | 0 | 0 | 0 | 0 | 0 | 0 | 23.405 |
| ASTRO2000480 | 0 | 0 | 0 | 6.823 | 0 | 0 | 0 |
| ASTRO2000801 | 0 | 0 | 0 | 0 | 0 | 0 | 1.267 |
| ASTRO2000914 | 0 | 3.537 | 0 | 0 | 0 | 0 | 0 |
| ASTRO2001029 | 0 | 2.279 | 0 | 12.089 | 0 | 0 | 1.558 |
| ASTRO2001076 | 0 | 0 | 0 | 3.426 | 0 | 0 | 5.297 |
| ASTRO2001107 | 0 | 0 | 0 | 0 | 0 | 0 | 9.074 |
| ASTRO2001823 | 0 | 0 | 0 | 3.498 | 0 | 0 | 0 |
| ASTRO2002202 | 0 | 0 | 0 | 5.554 | 0 | 0 | 0 |
| ASTRO2002693 | 25.689 | 0 | 0 | 0 | 0 | 0 | 0 |
| ASTRO2003316 | 0 | 10.721 | 0 | 0 | 0 | 0 | 0 |
| ASTRO2003461 | 0 | 0 | 0 | 0 | 0 | 0 | 14.800 |
| ASTRO2003632 | 0 | 0.755 | 0 | 1.001 | 0 | 0 | 0 |
| ASTRO2004032 | 0 | 0 | 0 | 0 | 0 | 0 | 15.142 |
| ASTRO2004974 | 0 | 0 | 0 | 0 | 0 | 0 | 4.828 |
| ASTRO2005423 | 12.479 | 0 | 0 | 8.299 | 0 | 0 | 12.832 |
| ASTRO2005553 | 0 | 0 | 0 | 0 | 0 | 0 | 6.438 |
| ASTRO2005687 | 0 | 0 | 0 | 0 | 0 | 0 | 1.385 |
| ASTRO2008216 | 0 | 0 | 0 | 0 | 0 | 0 | 3.964 |
| ASTRO2008409 | 0 | 23.625 | 0 | 31.333 | 0 | 0 | 0 |
| ASTRO2009068 | 0 | 0 | 0 | 0 | 0 | 0 | 7.029 |
| ASTRO2009118 | 0 | 28.385 | 0 | 0 | 0 | 0 | 0 |
| ASTRO2010819 | 0 | 0 | 0 | 1.622 | 0 | 0 | 6.687 |
| ASTRO2010996 | 0 | 34.405 | 0 | 0 | 0 | 0 | 0 |
| ASTRO2011422 | 0 | 0 | 0 | 17.485 | 0 | 0 | 18.025 |
| ASTRO2012552 | 5.357 | 2.686 | 0 | 0 | 0 | 0 | 0 |
| ASTRO2013585 | 3.533 | 1.772 | 0 | 2.350 | 0 | 0 | 0 |
| ASTRO2015529 | 0 | 0 | 0 | 0 | 0 | 13.655 | 0 |
| ASTRO2016148 | 0 | 0 | 0 | 0 | 0 | 0 | 26.393 |
| ASTRO2016491 | 0 | 0 | 0 | 3.641 | 0 | 0 | 1.877 |
| ASTRO2016819 | 0 | 23.453 | 0 | 0 | 0 | 0 | 0 |
| BGGI12000054 | 0 | 0.856 | 0 | 1.135 | 0 | 3.472 | 0 |
| BGGI12000533 | 0 | 0 | 0 | 0 | 0 | 0 | 2.805 |
| BGGI12000616 | 0 | 0 | 0 | 2.155 | 0 | 0 | 0 |
| BGGI12000684 | 0 | 4.483 | 0 | 0 | 0 | 18.183 | 0 |
| BGGI12000839 | 0 | 0 | 0 | 0 | 0 | 0 | 1.832 |
| BGGI12001002 | 0 | 0 | 0 | 0 | 0 | 13.083 | 0 |
| BGGI12001075 | 0 | 7.194 | 0 | 0 | 0 | 0 | 2.459 |
| BGGI12001097 | 0 | 2.042 | 0 | 0 | 0 | 0 | 0 |
| BGGI12001682 | 0 | 0 | 0 | 0 | 0 | 0 | 1.283 |
| BLADE2004089 | 0 | 0 | 0 | 0 | 0 | 0 | 12.311 |
| BLADE2004849 | 26.862 | 0 | 0 | 0 | 0 | 0 | 0 |
| BLADE2006830 | 0 | 0 | 0 | 0 | 0 | 24.778 | 0 |
| BLADE2008461 | 0 | 0 | 0 | 5.974 | 0 | 0 | 0 |
| BNGH41000098 | 0 | 0 | 0 | 0 | 0 | 0 | 3.149 |
| BNGH41000137 | 0 | 0 | 0 | 0 | 0 | 0 | 5.390 |
| BNGH41000153 | 0 | 8.539 | 0 | 11.325 | 0 | 0 | 0 |
| BNGH41000198 | 0 | 15.102 | 0 | 0 | 0 | 0 | 0 |
| BNGH42000360 | 0.570 | 0.286 | 0 | 0 | 0 | 0 | 0 |
| BNGH42000739 | 0 | 0 | 0 | 2.999 | 0 | 0 | 1.546 |
| BNGH42002168 | 0 | 0 | 38.287 | 0 | 0 | 9.806 | 0 |
| BNGH42006135 | 2.393 | 1.200 | 0 | 6.365 | 0 | 0 | 3.281 |
| BNGH42007460 | 0 | 1.831 | 0 | 0 | 0 | 0 | 1.252 |
| BNGH42007709 | 0.834 | 0 | 0 | 0 | 0 | 0 | 0 |
| BNGH42007788 | 0 | 0 | 0 | 7.419 | 0 | 0 | 0 |
| BNGH42007798 | 0 | 17.846 | 0 | 0 | 0 | 0 | 0 |
| BNGH42008603 | 5.242 | 0 | 0 | 0 | 0 | 0 | 5.390 |
| BNGH42008743 | 0 | 0 | 0 | 3.710 | 0 | 0 | 1.912 |
| BNGH42009025 | 5.052 | 0 | 0 | 0 | 0 | 0 | 0 |
| BRACE1000020 | 40.792 | 0 | 0 | 27.128 | 0 | 0 | 0 |
| BRACE1000042 | 0 | 0 | 0 | 0 | 0 | 0 | 1.891 |
| BRACE1000070 | 0 | 0 | 0 | 8.617 | 0 | 13.175 | 2.221 |
| BRACE1000087 | 0 | 0 | 0 | 35.366 | 0 | 0 | 0 |
| BRACE1000115 | 1.357 | 0 | 0 | 0 | 0 | 0 | 0 |
| BRACE1000131 | 3.775 | 0 | 0 | 2.510 | 0 | 0 | 1.294 |
| BRACE1000186 | 7.836 | 0 | 0 | 5.211 | 0 | 15.935 | 0 |
| BRACE1000187 | 0 | 0 | 0 | 0 | 0 | 0 | 23.470 |
| BRACE2000307 | 0 | 4.281 | 0 | 8.516 | 0 | 0 | 10.243 |
| BRACE2000332 | 0 | 0 | 0 | 0 | 0 | 0 | 3.372 |
| BRACE2000347 | 9.369 | 0 | 0 | 6.230 | 0 | 0 | 0 |
| BRACE2000487 | 29.738 | 0 | 0 | 0 | 0 | 0 | 0 |
| BRACE2000525 | 0 | 0 | 0 | 0 | 0 | 0 | 2.018 |
| BRACE2000698 | 0 | 0 | 0 | 0 | 0 | 63.412 | 0 |
| BRACE2000733 | 0 | 0 | 0 | 0 | 0 | 0 | 21.894 |
| BRACE2000975 | 2.322 | 0 | 0 | 1.544 | 0 | 0 | 7.958 |
| BRACE2001107 | 0 | 26.748 | 0 | 0 | 0 | 0 | 0 |
| BRACE2001143 | 0 | 2.700 | 0 | 0 | 0 | 0 | 0 |
| BRACE2001151 | 46.787 | 0 | 0 | 0 | 0 | 0 | 0 |
| BRACE2001236 | 0 | 0 | 0 | 0 | 0 | 0 | 22.352 |
| BRACE2001477 | 0 | 12.500 | 0 | 0 | 0 | 25.347 | 0 |
| BRACE2001497 | 0 | 3.386 | 0 | 0 | 0 | 0 | 11.574 |
| BRACE2001508 | 0 | 0 | 0 | 21.242 | 0 | 0 | 0 |
| BRACE2001692 | 0 | 0 | 0 | 1.459 | 0 | 0 | 0 |
| BRACE2001855 | 4.170 | 4.182 | 0 | 0 | 0 | 0 | 0 |
| BRACE2001881 | 83.623 | 0 | 0 | 0 | 0 | 0 | 0 |
| BRACE2001892 | 2.196 | 1.651 | 0 | 2.920 | 0 | 0 | 7.526 |
| BRACE2001944 | 0 | 0 | 0 | 0 | 0 | 0 | 15.586 |
| BRACE2001971 | 8.129 | 0 | 0 | 0 | 0 | 0 | 2.786 |
| BRACE2002050 | 0 | 0 | 0 | 0 | 0 | 0 | 3.602 |
| BRACE2002091 | 7.653 | 0 | 0 | 0 | 0 | 0 | 0 |
| BRACE2002356 | 0 | 0 | 0 | 43.792 | 0 | 0 | 0 |
| BRACE2002441 | 0 | 2.047 | 0 | 0 | 0 | 0 | 1.399 |
| BRACE2002582 | 0 | 23.746 | 0 | 0 | 0 | 0 | 0 |
| BRACE2002649 | 0 | 0 | 0 | 0 | 0 | 0 | 13.153 |
| BRACE2002677 | 0 | 0 | 0 | 0 | 0 | 0 | 2.398 |
| BRACE2002796 | 0 | 0 | 0 | 0 | 0 | 0 | 63.640 |
| BRACE2002803 | 0 | 0 | 0 | 0 | 0 | 0 | 22.636 |
| BRACE2002861 | 0 | 0 | 0 | 0 | 0 | 0 | 6.448 |
| BRACE2003037 | 0 | 0 | 0 | 0 | 0 | 0 | 5.365 |
| BRACE2003110 | 48.819 | 0 | 0 | 0 | 0 | 0 | 16.734 |
| BRACE2003172 | 0 | 0 | 0 | 0 | 0 | 0 | 17.361 |
| BRACE2003254 | 3.281 | 0 | 0 | 2.182 | 0 | 0 | 5.623 |
| BRACE2003304 | 0 | 19.864 | 0 | 10.538 | 0 | 0 | 2.716 |
| BRACE2003319 | 0 | 0 | 65.930 | 0 | 0 | 0 | 0 |
| BRACE2003527 | 9.342 | 0 | 0 | 0 | 0 | 0 | 0 |
| BRACE2003609 | 32.942 | 0 | 0 | 21.907 | 0 | 0 | 11.292 |
| BRACE2003628 | 0 | 0 | 0 | 16.442 | 0 | 0 | 0 |
| BRACE2003785 | 7.043 | 3.532 | 0 | 2.342 | 0 | 0 | 1.207 |
| BRACE2003827 | 15.876 | 4.776 | 0 | 0 | 0 | 0 | 0 |
| BRACE2003845 | 0 | 0 | 0 | 10.981 | 0 | 0 | 5.660 |
| BRACE2003846 | 0 | 1.184 | 0 | 3.141 | 0 | 0 | 3.238 |
| BRACE2003892 | 0 | 0 | 0 | 0 | 0 | 0 | 5.422 |
| BRACE2003904 | 0 | 4.711 | 0 | 6.248 | 0 | 0 | 0 |
| BRACE2003905 | 0 | 0 | 0 | 0 | 0 | 0 | 39.290 |
| BRACE2003944 | 0 | 0.941 | 0 | 0 | 0 | 34.336 | 0 |
| BRACE2005087 | 0 | 3.770 | 0 | 2.500 | 0 | 0 | 9.020 |
| BRACE2005151 | 7.615 | 0 | 0 | 0 | 0 | 0 | 0 |
| BRACE2005189 | 0 | 46.047 | 0 | 0 | 0 | 0 | 0 |
| BRACE2005196 | 0 | 0 | 0 | 0 | 0 | 0 | 39.504 |
| BRACE2005395 | 34.170 | 0 | 0 | 0 | 0 | 0 | 11.713 |
| BRACE2005434 | 2.499 | 0 | 0 | 0 | 0 | 0 | 0.856 |
| BRACE2005681 | 0 | 0 | 0 | 0 | 0 | 0 | 4.961 |
| BRACE2005719 | 10.763 | 0 | 0 | 0 | 0 | 0 | 0 |
| BRACE2005875 | 0 | 0 | 0 | 0 | 0 | 0 | 5.348 |
| BRACE2005937 | 0 | 0 | 0 | 0 | 0 | 0 | 7.504 |
| BRACE2006055 | 14.499 | 0 | 0 | 0 | 0 | 0 | 2.485 |
| BRACE2006162 | 0 | 0 | 0 | 0 | 0 | 11.404 | 0 |
| BRACE2006258 | 2.969 | 5.954 | 0 | 0 | 0 | 0 | 0 |
| BRACE2006281 | 0 | 0 | 86.959 | 0 | 0 | 0 | 0 |
| BRACE2006319 | 0 | 0 | 0 | 8.450 | 0 | 0 | 8.711 |
| BRACE2006354 | 0 | 0 | 0 | 0 | 0 | 0 | 23.191 |
| BRACE2006514 | 0 | 7.894 | 0 | 0 | 0 | 0 | 2.158 |
| BRACE2006636 | 0 | 0 | 0 | 0 | 0 | 0 | 17.031 |
| BRACE2006743 | 0 | 0 | 0 | 0 | 0 | 0 | 11.047 |
| BRACE2006833 | 0 | 0 | 0 | 0 | 0 | 0 | 46.671 |
| BRACE2006859 | 0 | 0 | 0 | 0 | 0 | 74.880 | 0 |
| BRACE2006900 | 0 | 0.935 | 0 | 0 | 0 | 0 | 0 |
| BRACE2006937 | 4.850 | 0 | 0 | 0 | 0 | 0 | 9.976 |
| BRACE2007064 | 0 | 0 | 0 | 0 | 0 | 0 | 8.074 |
| BRACE2007089 | 0 | 1.539 | 0 | 0 | 0 | 0 | 0 |
| BRACE2007174 | 0 | 28.390 | 0 | 0 | 0 | 0 | 0 |
| BRACE2007401 | 0 | 3.321 | 0 | 0 | 0 | 0 | 2.270 |
| BRACE2007518 | 0 | 0 | 0 | 0 | 0 | 0 | 8.458 |
| BRACE2007577 | 0 | 0 | 0 | 0 | 0 | 0 | 4.866 |
| BRACE2007641 | 0 | 0 | 0 | 0 | 0 | 0 | 33.106 |
| BRACE2007671 | 5.603 | 2.810 | 0 | 3.726 | 0 | 0 | 0 |
| BRACE2007760 | 0 | 0 | 0 | 0 | 0 | 0 | 16.281 |
| BRACE2007768 | 0 | 0 | 0 | 0 | 0 | 0 | 11.887 |
| BRACE2007902 | 0 | 0 | 0 | 77.250 | 0 | 0 | 0 |
| BRACE2007937 | 0 | 4.615 | 0 | 0 | 0 | 0 | 25.242 |
| BRACE2007952 | 0 | 0 | 0 | 0 | 0 | 0 | 3.012 |
| BRACE2008358 | 0 | 0 | 0 | 0 | 0 | 0 | 5.630 |
| BRACE2008380 | 0 | 3.550 | 0 | 0 | 0 | 0 | 2.427 |
| BRACE2008402 | 0 | 0 | 0 | 0 | 0 | 0 | 5.209 |
| BRACE2008481 | 10.117 | 0 | 0 | 0 | 0 | 0 | 0 |
| BRACE2008653 | 1.911 | 3.834 | 0 | 2.542 | 0 | 0 | 3.276 |
| BRACE2008968 | 2.107 | 0 | 0 | 1.401 | 0 | 0 | 0 |
| BRACE2009037 | 5.291 | 1.061 | 0 | 0 | 0 | 0 | 0.363 |
| BRACE2009045 | 0 | 0 | 0 | 0 | 0 | 0 | 22.440 |
| BRACE2009291 | 0 | 0 | 0 | 3.188 | 0 | 9.749 | 0 |
| BRACE2009307 | 0 | 0 | 0 | 0 | 0 | 0 | 18.399 |
| BRACE2009558 | 10.420 | 0 | 0 | 6.930 | 0 | 21.191 | 0 |
| BRACE2010669 | 0 | 0 | 0 | 0 | 0 | 0 | 31.677 |
| BRACE2011265 | 0 | 0 | 0 | 0 | 0 | 0 | 4.608 |
| BRACE2012317 | 0 | 1.247 | 0 | 0 | 0 | 5.057 | 1.705 |
| BRACE2012528 | 0 | 0.862 | 0 | 1.143 | 0 | 0 | 3.535 |
| BRACE2012814 | 5.557 | 0 | 0 | 3.696 | 0 | 0 | 3.810 |
| BRACE2014306 | 17.640 | 0 | 0 | 0 | 0 | 0 | 0 |
| BRACE2014850 | 0 | 0 | 0 | 0 | 0 | 0 | 9.317 |
| BRACE2015058 | 0 | 0 | 0 | 10.567 | 0 | 0 | 5.447 |
| BRACE2015132 | 0 | 29.940 | 0 | 0 | 0 | 0 | 0 |
| BRACE2017574 | 0 | 9.310 | 0 | 0 | 0 | 0 | 12.729 |
| BRACE2018337 | 0 | 4.328 | 0 | 0 | 0 | 0 | 5.918 |
| BRACE2019147 | 0 | 0 | 0 | 5.162 | 0 | 0 | 2.660 |
| BRACE2021014 | 0 | 0 | 0 | 0 | 0 | 0 | 14.719 |
| BRACE2021541 | 0 | 28.929 | 0 | 0 | 0 | 0 | 0 |
| BRACE2022389 | 0 | 0 | 0 | 0 | 0 | 0 | 13.200 |
| BRACE2022549 | 0 | 0 | 0 | 0 | 0 | 0 | 63.640 |
| BRACE2022638 | 0 | 2.709 | 0 | 3.593 | 0 | 0 | 2.778 |
| BRACE2025492 | 0 | 0 | 0 | 0 | 0 | 0 | 32.696 |
| BRACE2026054 | 0 | 41.542 | 0 | 0 | 0 | 0 | 0 |
| BRACE2026725 | 66.449 | 0 | 0 | 0 | 0 | 0 | 0 |
| BRACE2027258 | 0 | 0 | 0 | 0 | 0 | 0 | 41.974 |
| BRACE2027643 | 0 | 0 | 0 | 0 | 0 | 0 | 17.893 |
| BRACE2032477 | 0 | 0 | 0 | 0 | 0 | 0 | 20.768 |
| BRACE2035278 | 0 | 0 | 0 | 0 | 0 | 0 | 46.671 |
| BRACE2037295 | 23.776 | 0 | 0 | 0 | 0 | 0 | 0 |
| BRACE2037310 | 0 | 0 | 0 | 0 | 0 | 0 | 48.748 |
| BRACE2039624 | 0 | 3.516 | 0 | 4.663 | 0 | 0 | 2.404 |
| BRACE2044286 | 0 | 33.923 | 0 | 0 | 0 | 0 | 0 |
| BRACE2045954 | 0 | 0 | 0 | 0 | 0 | 0 | 33.925 |
| BRACE3001113 | 0 | 0 | 0 | 0 | 0 | 0 | 32.696 |
| BRACE3001973 | 0 | 0 | 0 | 0 | 0 | 0 | 63.640 |
| BRACE3002184 | 0 | 0 | 0 | 0 | 0 | 0 | 33.290 |
| BRACE3003026 | 0 | 59.349 | 0 | 0 | 0 | 0 | 0 |
| BRACE3003053 | 0 | 0 | 0 | 0 | 0 | 0 | 63.640 |
| BRACE3004371 | 0 | 0 | 0 | 0 | 0 | 0 | 9.334 |
| BRACE3005938 | 0 | 2.001 | 0 | 2.654 | 0 | 0 | 1.368 |
| BRACE3010428 | 0 | 2.080 | 0 | 0 | 0 | 0 | 4.266 |
| BRACE3011271 | 0 | 0 | 0 | 0 | 0 | 0 | 27.420 |
| BRACE3013780 | 0 | 0 | 0 | 0 | 0 | 0 | 5.958 |
| BRACE3027931 | 0 | 0 | 0 | 0 | 0 | 0 | 41.796 |
| BRACE3030538 | 0 | 0 | 0 | 0 | 0 | 0 | 63.640 |
| BRALZ2009482 | 0 | 2.026 | 0 | 0 | 0 | 2.739 | 0 |
| BRAMY1000173 | 0 | 0 | 0 | 0 | 0 | 0 | 26.275 |
| BRAMY2000354 | 4.980 | 0 | 0 | 0 | 0 | 0 | 0 |
| BRAMY2000562 | 0 | 0 | 0 | 0 | 0 | 0 | 4.775 |
| BRAMY2004363 | 0 | 3.715 | 0 | 0 | 0 | 0 | 10.159 |
| BRAMY2007567 | 0 | 0 | 0 | 0 | 0 | 0 | 9.640 |
| BRAMY2007613 | 0 | 4.869 | 0 | 0 | 0 | 0 | 8.321 |
| BRAMY2008382 | 56.197 | 28.179 | 0 | 0 | 0 | 0 | 0 |
| BRAMY2009013 | 0 | 11.068 | 0 | 14.679 | 0 | 0 | 52.965 |
| BRAMY2009508 | 0 | 13.626 | 0 | 0 | 0 | 0 | 0 |
| BRAMY2010357 | 0 | 0 | 0 | 0 | 0 | 0 | 18.549 |
| BRAMY2010464 | 0 | 40.041 | 0 | 0 | 0 | 0 | 0 |
| BRAMY2012091 | 0 | 0 | 0 | 0 | 0 | 0 | 7.377 |
| BRAMY2012581 | 0 | 0 | 0 | 0 | 0 | 0 | 28.378 |
| BRAMY2013169 | 0 | 0 | 0 | 0 | 0 | 0 | 14.126 |
| BRAMY2013621 | 0 | 0 | 0 | 70.518 | 0 | 0 | 0 |
| BRAMY2013659 | 0 | 0 | 0 | 0 | 0 | 0 | 36.086 |
| BRAMY2013756 | 0 | 68.630 | 0 | 0 | 0 | 0 | 0 |
| BRAMY2014205 | 0 | 0 | 0 | 0 | 0 | 0 | 17.799 |
| BRAMY2015251 | 0 | 0 | 0 | 0 | 0 | 0 | 7.608 |
| BRAMY2015705 | 0 | 0 | 0 | 70.518 | 0 | 0 | 0 |
| BRAMY2016009 | 0 | 0 | 0 | 12.604 | 0 | 0 | 6.496 |
| BRAMY2016251 | 0 | 0 | 0 | 0 | 0 | 0 | 8.934 |
| BRAMY2016706 | 0 | 9.007 | 0 | 0 | 0 | 0 | 0 |
| BRAMY2018785 | 0 | 0 | 0 | 16.390 | 0 | 0 | 0 |
| BRAMY2019554 | 2.892 | 1.450 | 0 | 3.847 | 0 | 0 | 15.862 |
| BRAMY2019600 | 0 | 0 | 0 | 0 | 0 | 0 | 30.091 |
| BRAMY2019963 | 0 | 0 | 0 | 0 | 0 | 0 | 25.378 |
| BRAMY2020106 | 0 | 0 | 0 | 0 | 0 | 0 | 27.775 |
| BRAMY2022493 | 0 | 0 | 0 | 6.394 | 0 | 0 | 3.296 |
| BRAMY2022525 | 0 | 40.075 | 0 | 0 | 0 | 0 | 0 |
| BRAMY2023863 | 0 | 64.331 | 0 | 0 | 0 | 0 | 0 |
| BRAMY2023939 | 22.151 | 11.107 | 0 | 0 | 0 | 0 | 15.186 |
| BRAMY2027587 | 0 | 0 | 0 | 0 | 0 | 0 | 16.588 |
| BRAMY2028269 | 0 | 29.661 | 0 | 0 | 0 | 0 | 0 |
| BRAMY2028282 | 0 | 0 | 0 | 0 | 0 | 0 | 30.319 |
| BRAMY2028740 | 0 | 1.692 | 0 | 2.244 | 0 | 0 | 0 |
| BRAMY2028796 | 0 | 0 | 0 | 0 | 0 | 0 | 3.693 |
| BRAMY2029988 | 0 | 0 | 0 | 0 | 0 | 0 | 14.633 |
| BRAMY2031516 | 0 | 0 | 0 | 17.802 | 0 | 0 | 0 |
| BRAMY2032087 | 0 | 0 | 0 | 0 | 0 | 0 | 6.018 |
| BRAMY2036459 | 0 | 0 | 0 | 0 | 0 | 0 | 2.341 |
| BRAMY2040095 | 1.239 | 1.864 | 0 | 2.472 | 0 | 0 | 0 |
| BRAMY3002991 | 14.210 | 0 | 0 | 0 | 0 | 0 | 0 |
| BRAMY3003026 | 0 | 0 | 0 | 0 | 0 | 0 | 55.216 |
| BRAMY3004377 | 9.017 | 1.507 | 0 | 3.998 | 0 | 6.112 | 2.061 |
| BRAMY3005184 | 0 | 0 | 0 | 0 | 0 | 0 | 27.836 |
| BRAMY3005912 | 0 | 0 | 0 | 0 | 0 | 0 | 26.605 |
| BRAMY3008044 | 0 | 3.260 | 0 | 0 | 0 | 0 | 20.056 |
| BRAMY3010008 | 0 | 0 | 0 | 0 | 0 | 4.289 | 0 |
| BRAMY4002628 | 0 | 12.908 | 0 | 17.119 | 0 | 0 | 8.824 |
| BRAWH1000007 | 0 | 0 | 0 | 21.657 | 0 | 0 | 11.163 |
| BRAWH1000037 | 0 | 3.314 | 0 | 4.395 | 0 | 0 | 0 |
| BRAWH1000040 | 0 | 1.250 | 0 | 1.658 | 0 | 0 | 5.129 |
| BRAWH1000045 | 3.628 | 1.819 | 0 | 0 | 0 | 0 | 4.974 |
| BRAWH1000093 | 0 | 0 | 0 | 0 | 0 | 0 | 2.292 |
| BRAWH1000127 | 0 | 0 | 0 | 0 | 0 | 0 | 3.971 |
| BRAWH1000162 | 0 | 0 | 0 | 0 | 0 | 0 | 9.069 |
| BRAWH2000082 | 0 | 1.394 | 0 | 5.547 | 0 | 0 | 0 |
| BRAWH2000274 | 0 | 0 | 0 | 0 | 0 | 0 | 7.357 |
| BRAWH2000460 | 0 | 5.545 | 0 | 1.471 | 0 | 0 | 1.516 |
| BRAWH2000488 | 0 | 18.321 | 0 | 0 | 0 | 0 | 12.525 |
| BRAWH2000522 | 13.137 | 0 | 0 | 0 | 0 | 0 | 2.252 |
| BRAWH2000588 | 4.547 | 2.280 | 0 | 0 | 0 | 0 | 1.559 |
| BRAWH2000633 | 0 | 0 | 0 | 0 | 0 | 0 | 4.941 |
| BRAWH2000636 | 1.915 | 0 | 0 | 5.094 | 0 | 0 | 5.908 |
| BRAWH2000686 | 0 | 0 | 0 | 0 | 0 | 0 | 10.630 |
| BRAWH2000697 | 3.666 | 1.838 | 0 | 2.438 | 0 | 7.455 | 0 |
| BRAWH2000839 | 1.488 | 2.239 | 0 | 1.980 | 0 | 0 | 0.510 |
| BRAWH2000924 | 0 | 0 | 0 | 0 | 0 | 0 | 0.713 |
| BRAWH2000944 | 0 | 9.480 | 0 | 0 | 0 | 0 | 0 |
| BRAWH2001037 | 0 | 0 | 0 | 0 | 0 | 0 | 24.911 |
| BRAWH2001092 | 0 | 2.555 | 0 | 0.847 | 0 | 0 | 2.838 |
| BRAWH2001141 | 0 | 0 | 0 | 0 | 0 | 0 | 6.313 |
| BRAWH2001166 | 0 | 5.736 | 0 | 22.824 | 0 | 0 | 3.921 |
| BRAWH2001171 | 0 | 2.245 | 0 | 8.931 | 0 | 0 | 15.345 |
| BRAWH2001186 | 0 | 0 | 81.991 | 0 | 0 | 0 | 0 |
| BRAWH2001321 | 6.222 | 1.560 | 0 | 0 | 0 | 0 | 7.465 |
| BRAWH2001412 | 54.493 | 0 | 0 | 0 | 0 | 0 | 0 |
| BRAWH2001424 | 0 | 0 | 0 | 0 | 0 | 37.524 | 0 |
| BRAWH2001439 | 3.729 | 1.870 | 0 | 4.960 | 0 | 0 | 7.669 |
| BRAWH2001484 | 0 | 0 | 0 | 0 | 0 | 0 | 13.199 |
| BRAWH2001530 | 0 | 0 | 0 | 0 | 0 | 0 | 9.483 |
| BRAWH2001662 | 0 | 0 | 0 | 22.162 | 0 | 0 | 5.712 |
| BRAWH2001862 | 0 | 0 | 96.642 | 0 | 0 | 0 | 0 |
| BRAWH2002560 | 0 | 0 | 0 | 0 | 0 | 0 | 24.052 |
| BRAWH2002811 | 0 | 15.672 | 0 | 0 | 0 | 0 | 0 |
| BRAWH2002967 | 0 | 0 | 0 | 0 | 0 | 0 | 55.403 |
| BRAWH2003662 | 0 | 0 | 0 | 0 | 0 | 0 | 7.699 |
| BRAWH2003693 | 0 | 12.434 | 0 | 0 | 0 | 0 | 0 |
| BRAWH2003818 | 0 | 0 | 0 | 7.294 | 0 | 0 | 11.280 |
| BRAWH2004095 | 0 | 0 | 0 | 5.698 | 0 | 0 | 7.343 |
| BRAWH2005225 | 0 | 0 | 0 | 0 | 0 | 0 | 21.603 |
| BRAWH2005517 | 0 | 0 | 0 | 0 | 0 | 0 | 55.403 |
| BRAWH2005661 | 0 | 0 | 0 | 0 | 0 | 0 | 3.972 |
| BRAWH2006044 | 0 | 31.972 | 0 | 0 | 0 | 0 | 0 |
| BRAWH2006622 | 59.966 | 0 | 0 | 0 | 0 | 0 | 0 |
| BRAWH2007605 | 0 | 7.455 | 0 | 9.887 | 0 | 0 | 2.548 |
| BRAWH2007664 | 37.081 | 0 | 0 | 0 | 0 | 0 | 0 |
| BRAWH2007800 | 0 | 0 | 0 | 0 | 0 | 0 | 2.918 |
| BRAWH2008058 | 0 | 0 | 0 | 0 | 0 | 0 | 29.101 |
| BRAWH2008255 | 15.311 | 0 | 0 | 0 | 0 | 0 | 5.248 |
| BRAWH2008706 | 0 | 0 | 0 | 0 | 0 | 0 | 3.572 |
| BRAWH2009626 | 0 | 0 | 0 | 0 | 0 | 0 | 55.403 |
| BRAWH2010329 | 0 | 0 | 0 | 0 | 0 | 0 | 6.217 |
| BRAWH2010754 | 40.540 | 0 | 0 | 0 | 0 | 0 | 0 |
| BRAWH2010980 | 25.609 | 0 | 0 | 0 | 0 | 0 | 0 |
| BRAWH2011286 | 0 | 0 | 0 | 21.508 | 0 | 0 | 0 |
| BRAWH2011823 | 78.374 | 0 | 0 | 0 | 0 | 0 | 0 |
| BRAWH2011920 | 0 | 0 | 0 | 0 | 0 | 0 | 23.538 |
| BRAWH2012164 | 0 | 0 | 0 | 0 | 0 | 0 | 25.884 |
| BRAWH2012866 | 0 | 0 | 0 | 0 | 0 | 0 | 18.485 |
| BRAWH2014188 | 0 | 5.204 | 0 | 0 | 0 | 0 | 2.668 |
| BRAWH2014379 | 0 | 37.724 | 0 | 0 | 0 | 0 | 0 |
| BRAWH2014645 | 0 | 1.921 | 0 | 7.643 | 0 | 0 | 6.566 |
| BRAWH2014662 | 56.250 | 0 | 0 | 0 | 0 | 0 | 0 |
| BRAWH2016446 | 0 | 0 | 0 | 20.968 | 0 | 0 | 10.808 |
| BRAWH2017002 | 0 | 0 | 0 | 0 | 0 | 0 | 12.365 |
| BRAWH2017304 | 0 | 0 | 0 | 40.020 | 0 | 0 | 0 |
| BRAWH2017685 | 1.839 | 0 | 0 | 3.669 | 0 | 3.740 | 5.044 |
| BRAWH2018526 | 0 | 0.559 | 0 | 0 | 0 | 0 | 0 |
| BRAWH3000088 | 4.192 | 8.409 | 0 | 2.788 | 0 | 0 | 4.311 |
| BRAWH3001918 | 0 | 0 | 0 | 0 | 0 | 0 | 17.250 |
| BRAWH3002574 | 0 | 12.014 | 0 | 0 | 0 | 0 | 8.213 |
| BRAWH3002853 | 0 | 1.344 | 0 | 5.348 | 0 | 0 | 1.838 |
| BRAWH3003727 | 0 | 0 | 0 | 0 | 0 | 0 | 12.492 |
| BRAWH3004222 | 0 | 0 | 0 | 0 | 0 | 0 | 55.403 |
| BRAWH3004350 | 0 | 0 | 0 | 0 | 0 | 0 | 20.597 |
| BRAWH3005981 | 0 | 0 | 0 | 70.676 | 0 | 0 | 0 |
| BRAWH3008931 | 0 | 0 | 0 | 0 | 0 | 0 | 4.303 |
| BRAWH3009546 | 0 | 0 | 0 | 0 | 0 | 0 | 29.283 |
| BRAWH3010461 | 0 | 3.733 | 0 | 0 | 0 | 0 | 15.313 |
| BRAWH3014609 | 0 | 0 | 0 | 0 | 0 | 0 | 55.403 |
| BRAWH3017259 | 50.722 | 0 | 0 | 0 | 0 | 0 | 17.387 |
| BRAWH3018063 | 0 | 14.389 | 0 | 0 | 0 | 0 | 3.279 |
| BRAWH3021545 | 0 | 0 | 0 | 0 | 0 | 26.887 | 0 |
| BRAWH3021574 | 0 | 0 | 0 | 0 | 0 | 0 | 11.677 |
| BRAWH3027533 | 0 | 0 | 0 | 0 | 0 | 0 | 13.675 |
| BRAWH3029806 | 0 | 0 | 0 | 0 | 0 | 0 | 22.142 |
| BRAWH3034743 | 0 | 0 | 0 | 0 | 0 | 0 | 10.350 |
| BRAWH3037265 | 0 | 0 | 0 | 0 | 0 | 0 | 55.403 |
| BRCAN1000168 | 0 | 0 | 0 | 0 | 0 | 10.397 | 0 |
| BRCAN2000563 | 0 | 23.897 | 0 | 0 | 0 | 0 | 0 |
| BRCAN2003389 | 0 | 5.526 | 87.498 | 0 | 0 | 0 | 0 |
| BRCAN2003944 | 1.021 | 0 | 0 | 2.036 | 0 | 0 | 0 |
| BRCAN2007409 | 0 | 0 | 0 | 0 | 0 | 0 | 3.727 |
| BRCAN2009432 | 0 | 0 | 0 | 3.173 | 0 | 0 | 0 |
| BRCAN2016619 | 0 | 23.940 | 0 | 0 | 0 | 0 | 0 |
| BRCAN2019653 | 0 | 0 | 0 | 15.031 | 0 | 0 | 0 |
| BRCAN2022126 | 0 | 8.824 | 0 | 0 | 0 | 0 | 0 |
| BRCAN2026197 | 0 | 0 | 0 | 0 | 0 | 0 | 1.940 |
| BRCAN2027364 | 0 | 0 | 0 | 0 | 0 | 0 | 7.379 |
| BRCAN2028036 | 0 | 4.387 | 0 | 0 | 0 | 0 | 0 |
| BRCAN2028355 | 5.354 | 0 | 0 | 0 | 0 | 0 | 0 |
| BRCOC1000040 | 0 | 0 | 0 | 0 | 0 | 0 | 3.664 |
| BRCOC2000152 | 0 | 0 | 0 | 0 | 0 | 0 | 25.963 |
| BRCOC2000184 | 0 | 0 | 0 | 0 | 0 | 61.762 | 0 |
| BRCOC2000487 | 0 | 0.526 | 0 | 0 | 95.528 | 0 | 0 |
| BRCOC2010123 | 3.970 | 3.981 | 0 | 2.640 | 0 | 0 | 0 |
| BRCOC2010510 | 0 | 0 | 0 | 0 | 0 | 0 | 14.918 |
| BRCOC2012172 | 0 | 5.112 | 0 | 13.559 | 0 | 0 | 0 |
| BRCOC2012386 | 0 | 20.721 | 0 | 27.482 | 0 | 0 | 0 |
| BRCOC2014400 | 0 | 0 | 0 | 40.488 | 0 | 0 | 0 |
| BRCOC2019910 | 0 | 0 | 0 | 0 | 0 | 0 | 2.329 |
| BRHIP1000104 | 2.369 | 0 | 0 | 0 | 0 | 0 | 0 |
| BRHIP1000108 | 0 | 0 | 0 | 0 | 0 | 0 | 5.354 |
| BRHIP1000174 | 0 | 0 | 0 | 0 | 0 | 0 | 6.298 |
| BRHIP2000312 | 0 | 0 | 0 | 0 | 0 | 0 | 8.056 |
| BRHIP2000534 | 1.240 | 1.244 | 0 | 0.825 | 0 | 0 | 0 |
| BRHIP2000553 | 0 | 9.340 | 0 | 12.387 | 0 | 0 | 12.769 |
| BRHIP2001074 | 0 | 0 | 0 | 47.218 | 0 | 0 | 0 |
| BRHIP2007305 | 0 | 0 | 0 | 0 | 0 | 0 | 2.813 |
| BRHIP2007741 | 6.291 | 6.309 | 0 | 0 | 0 | 0 | 4.313 |
| BRHIP2010217 | 0 | 0 | 0 | 0 | 0 | 0 | 6.938 |
| BRHIP2012627 | 0 | 28.546 | 0 | 0 | 0 | 0 | 0 |
| BRHIP2013286 | 0 | 0 | 0 | 82.532 | 0 | 0 | 0 |
| BRHIP2014228 | 0 | 0 | 0 | 35.475 | 0 | 0 | 0 |
| BRHIP2015224 | 0 | 0 | 0 | 0 | 0 | 0 | 11.303 |
| BRHIP2015245 | 0 | 0 | 0 | 0 | 0 | 0 | 14.746 |
| BRHIP2015679 | 9.091 | 0 | 0 | 0 | 0 | 18.488 | 0 |
| BRHIP2018712 | 0 | 0 | 0 | 0 | 0 | 0 | 7.547 |
| BRHIP2019819 | 0 | 0 | 0 | 0 | 0 | 0 | 10.771 |
| BRHIP2020573 | 0 | 0 | 0 | 0 | 0 | 0 | 22.485 |
| BRHIP2020799 | 0 | 9.197 | 0 | 0 | 0 | 0 | 0 |
| BRHIP2021762 | 0 | 5.063 | 0 | 0 | 0 | 0 | 3.461 |
| BRHIP2021858 | 0 | 0 | 0 | 0 | 0 | 0 | 8.314 |
| BRHIP2022228 | 0 | 0 | 0 | 0 | 0 | 0 | 11.352 |
| BRHIP2022326 | 0 | 1.546 | 0 | 4.101 | 0 | 0 | 1.057 |
| BRHIP2022986 | 0 | 0 | 0 | 0 | 0 | 0 | 10.604 |
| BRHIP2023762 | 0 | 0 | 0 | 5.752 | 0 | 0 | 0 |
| BRHIP2023888 | 2.667 | 7.355 | 0 | 2.660 | 0 | 0 | 3.200 |
| BRHIP2024398 | 0 | 0 | 0 | 0 | 0 | 0 | 10.459 |
| BRHIP2024742 | 0 | 0 | 0 | 12.023 | 0 | 0 | 9.296 |
| BRHIP2024911 | 0 | 67.372 | 0 | 0 | 0 | 0 | 0 |
| BRHIP2025448 | 0 | 0 | 0 | 0 | 0 | 0 | 37.989 |
| BRHIP2026061 | 0 | 0 | 0 | 0 | 0 | 0 | 37.989 |
| BRHIP2027017 | 0 | 20.920 | 0 | 0 | 0 | 0 | 14.301 |
| BRHIP2029643 | 0 | 0 | 0 | 0 | 0 | 0 | 18.557 |
| BRHIP2029663 | 0 | 0 | 0 | 0 | 0 | 0 | 54.907 |
| BRHIP3000017 | 0 | 8.013 | 0 | 0 | 0 | 0 | 5.478 |
| BRHIP3000111 | 3.710 | 1.860 | 0 | 0 | 0 | 0 | 0 |
| BRHIP3000131 | 0 | 6.673 | 0 | 0 | 0 | 0 | 0 |
| BRHIP3000457 | 0 | 0 | 0 | 0 | 0 | 0 | 33.739 |
| BRHIP3000475 | 0 | 0 | 0 | 0 | 0 | 0 | 53.721 |
| BRHIP3002691 | 0 | 0 | 0 | 0 | 0 | 0 | 10.303 |
| BRHIP3003795 | 0 | 0 | 0 | 0 | 0 | 0 | 54.907 |
| BRHIP3004968 | 0 | 0 | 0 | 0 | 0 | 0 | 41.796 |
| BRHIP3006717 | 6.168 | 0 | 0 | 0 | 0 | 0 | 8.458 |
| BRHIP3007223 | 0 | 3.664 | 0 | 4.859 | 0 | 14.859 | 2.505 |
| BRHIP3007586 | 12.507 | 0 | 0 | 0 | 0 | 0 | 0 |
| BRHIP3008082 | 0 | 0 | 0 | 0 | 0 | 0 | 54.907 |
| BRHIP3008320 | 0 | 0 | 0 | 0 | 0 | 0 | 29.007 |
| BRHIP3018784 | 0 | 0 | 0 | 0 | 0 | 0 | 4.305 |
| BRHIP3021019 | 0 | 14.074 | 0 | 0 | 0 | 0 | 28.863 |
| BRSSN2000498 | 0 | 1.989 | 0 | 0 | 0 | 0 | 0 |
| BRSSN2000561 | 0 | 0 | 0 | 15.113 | 0 | 0 | 7.790 |
| BRSSN2009518 | 12.485 | 0 | 0 | 4.151 | 0 | 0 | 0 |
| BRSSN2011653 | 0 | 0 | 0 | 0 | 0 | 0 | 24.622 |
| BRSSN2011738 | 0 | 0 | 0 | 0 | 0 | 0 | 13.066 |
| BRSSN2011843 | 0 | 0 | 0 | 0 | 0 | 0 | 14.868 |
| BRSSN2013753 | 0 | 26.797 | 0 | 0 | 0 | 0 | 18.319 |
| BRSSN2014610 | 0 | 0 | 0 | 17.209 | 0 | 0 | 0 |
| BRSSN2015238 | 0 | 0 | 0 | 0 | 0 | 0 | 8.505 |
| BRSSN2018440 | 0 | 0 | 0 | 0 | 0 | 0 | 25.025 |
| BRSTN1000083 | 0 | 3.481 | 0 | 0 | 0 | 4.706 | 4.759 |
| BRSTN2011961 | 3.149 | 0.526 | 8.334 | 0 | 0 | 2.134 | 0 |
| BRSTN2012069 | 0.788 | 0.052 | 0 | 0.479 | 0 | 0 | 0.047 |
| BRSTN2012174 | 1.466 | 1.470 | 5.820 | 0.487 | 0 | 0 | 0 |
| BRSTN2015889 | 0 | 7.039 | 0 | 0 | 0 | 0 | 0 |
| BRSTN2016992 | 0 | 0 | 0 | 0 | 0 | 0 | 0.611 |
| BRTHA1000311 | 0 | 0 | 0 | 0 | 0 | 0 | 7.331 |
| BRTHA2005864 | 0 | 0 | 0 | 0 | 0 | 0 | 6.047 |
| BRTHA2007998 | 0 | 0 | 0 | 0 | 0 | 0 | 2.143 |
| BRTHA2013054 | 0 | 0 | 0 | 0 | 0 | 0 | 7.462 |
| BRTHA2016577 | 0 | 4.288 | 0 | 0 | 0 | 17.389 | 2.931 |
| BRTHA2033155 | 0 | 7.729 | 0 | 0 | 0 | 0 | 21.134 |
| BRTHA3000633 | 0 | 62.102 | 0 | 0 | 0 | 0 | 0 |
| BRTHA3002427 | 0 | 0 | 0 | 0 | 0 | 0 | 3.551 |
| BRTHA3003225 | 0 | 0 | 0 | 0 | 0 | 0 | 52.835 |
| BRTHA3003490 | 0 | 0 | 0 | 3.913 | 0 | 11.964 | 8.067 |
| BRTHA3003736 | 0 | 0 | 0 | 0 | 0 | 0 | 8.017 |
| BRTHA3004307 | 0 | 0 | 0 | 0 | 0 | 0 | 11.227 |
| BRTHA3007472 | 0 | 7.091 | 0 | 0 | 0 | 0 | 4.847 |
| BRTHA3007922 | 0 | 0 | 0 | 37.613 | 0 | 0 | 0 |
| BRTHA3008778 | 0 | 0 | 0 | 0 | 0 | 0 | 30.863 |
| BRTHA3011194 | 12.809 | 0 | 0 | 0 | 0 | 0 | 13.172 |
| BRTHA3011265 | 0 | 0 | 0 | 20.563 | 0 | 0 | 0 |
| BRTHA3021971 | 0 | 0 | 55.855 | 0 | 0 | 0 | 2.411 |
| BRTHA3023403 | 0 | 0 | 0 | 0 | 0 | 0 | 27.684 |
| CD34C3000252 | 0 | 0 | 0 | 0 | 0 | 12.098 | 0 |
| CHONS2002829 | 0 | 2.969 | 0 | 0 | 0 | 0 | 0 |
| COLON2001721 | 0 | 10.053 | 0 | 0 | 0 | 0 | 0 |
| CTONG1000010 | 0 | 0 | 0 | 15.625 | 0 | 0 | 0 |
| CTONG1000052 | 0 | 1.098 | 0 | 11.650 | 0 | 4.453 | 6.005 |
| CTONG1000087 | 3.790 | 5.701 | 0 | 5.041 | 0 | 0 | 1.299 |
| CTONG1000094 | 0 | 1.415 | 0 | 0 | 0 | 0 | 4.354 |
| CTONG1000218 | 0 | 3.500 | 13.856 | 1.161 | 0 | 0 | 1.795 |
| CTONG1000241 | 0 | 0 | 0 | 0 | 0 | 0 | 7.906 |
| CTONG1000288 | 0 | 0 | 0 | 0 | 0 | 11.209 | 0 |
| CTONG2000034 | 0 | 0 | 0 | 0 | 0 | 66.426 | 0 |
| CTONG2000063 | 0 | 0 | 0 | 5.160 | 0 | 0 | 0 |
| CTONG2000452 | 0 | 0 | 0 | 0 | 0 | 0 | 3.618 |
| CTONG2000469 | 0 | 5.307 | 0 | 0 | 0 | 0 | 0 |
| CTONG2000508 | 0 | 1.435 | 0 | 1.903 | 0 | 0 | 3.924 |
| CTONG2001748 | 0 | 0 | 0 | 5.038 | 0 | 0 | 10.387 |
| CTONG2001820 | 0 | 49.384 | 0 | 0 | 0 | 0 | 0 |
| CTONG2001911 | 0 | 0 | 0 | 0 | 0 | 0 | 18.771 |
| CTONG2001932 | 0 | 0 | 0 | 0 | 0 | 0 | 12.834 |
| CTONG2002073 | 0 | 2.662 | 0 | 0 | 0 | 0 | 1.820 |
| CTONG2002095 | 0 | 12.727 | 0 | 33.758 | 0 | 0 | 8.700 |
| CTONG2002270 | 0 | 0.964 | 0 | 1.278 | 0 | 3.909 | 2.636 |
| CTONG2002709 | 0 | 0 | 62.349 | 0 | 0 | 0 | 0 |
| CTONG2002744 | 0 | 0 | 0 | 2.839 | 0 | 0 | 4.390 |
| CTONG2002803 | 0 | 10.703 | 0 | 3.549 | 0 | 0 | 1.829 |
| CTONG2002820 | 0 | 0 | 0 | 0 | 0 | 0 | 27.945 |
| CTONG2002832 | 0 | 0 | 0 | 0 | 0 | 0 | 27.853 |
| CTONG2002841 | 0 | 0 | 0 | 0 | 0 | 0 | 26.318 |
| CTONG2002903 | 0 | 5.598 | 0 | 0 | 0 | 0 | 0 |
| CTONG2003094 | 0 | 3.330 | 0 | 0 | 0 | 0 | 4.552 |
| CTONG2003293 | 0 | 0 | 0 | 22.278 | 0 | 0 | 11.483 |
| CTONG2003298 | 0 | 0 | 0 | 0 | 0 | 5.348 | 2.705 |
| CTONG2003524 | 0 | 2.928 | 0 | 0 | 0 | 11.875 | 4.003 |
| CTONG2004423 | 0 | 20.882 | 0 | 0 | 0 | 0 | 0 |
| CTONG2004487 | 0 | 0 | 0 | 2.280 | 0 | 6.971 | 3.525 |
| CTONG2005028 | 0 | 12.439 | 0 | 0 | 0 | 0 | 0 |
| CTONG2005278 | 10.444 | 0 | 0 | 6.946 | 0 | 0 | 21.481 |
| CTONG2005553 | 0 | 0 | 0 | 3.452 | 0 | 0 | 3.559 |
| CTONG2006004 | 0 | 1.918 | 0 | 2.544 | 0 | 0 | 2.622 |
| CTONG2006235 | 0 | 0 | 0 | 3.948 | 0 | 0 | 2.035 |
| CTONG2006412 | 0 | 0 | 0 | 14.961 | 0 | 0 | 15.423 |
| CTONG2006449 | 0 | 0 | 0 | 0 | 0 | 0 | 10.920 |
| CTONG2006562 | 0 | 0 | 0 | 0 | 0 | 10.318 | 3.479 |
| CTONG2006709 | 0 | 0 | 0 | 0 | 0 | 0 | 12.850 |
| CTONG2007078 | 0 | 0 | 0 | 0 | 0 | 0 | 1.588 |
| CTONG2007400 | 0 | 20.911 | 0 | 0 | 0 | 0 | 0 |
| CTONG2007500 | 0 | 1.367 | 0 | 1.812 | 0 | 0 | 0.934 |
| CTONG2007586 | 21.782 | 0 | 0 | 0 | 0 | 0 | 14.933 |
| CTONG2007959 | 0 | 66.117 | 0 | 0 | 0 | 0 | 0 |
| CTONG2008321 | 0 | 0 | 0 | 0 | 0 | 0 | 18.688 |
| CTONG2008398 | 0 | 2.779 | 0 | 0 | 0 | 0 | 1.900 |
| CTONG2008466 | 0 | 14.208 | 0 | 0 | 0 | 0 | 9.713 |
| CTONG2008506 | 0 | 0 | 0 | 4.536 | 0 | 0 | 5.845 |
| CTONG2008518 | 0 | 6.999 | 0 | 9.282 | 0 | 0 | 0 |
| CTONG2008792 | 0 | 13.353 | 0 | 0 | 0 | 0 | 9.128 |
| CTONG2009108 | 0 | 0 | 0 | 0 | 0 | 0 | 29.481 |
| CTONG2009529 | 22.915 | 0 | 0 | 0 | 0 | 0 | 0 |
| CTONG2010348 | 0 | 0 | 0 | 4.268 | 0 | 0 | 2.200 |
| CTONG2010566 | 0 | 24.642 | 0 | 0 | 0 | 0 | 0 |
| CTONG2010649 | 5.667 | 2.842 | 0 | 3.769 | 0 | 0 | 11.655 |
| CTONG2010821 | 0 | 0 | 0 | 1.722 | 0 | 0 | 0 |
| CTONG2011801 | 0 | 0 | 0 | 0 | 0 | 0 | 11.035 |
| CTONG2012452 | 0 | 3.236 | 0 | 0 | 0 | 0 | 1.327 |
| CTONG2012453 | 0 | 0 | 0 | 18.151 | 0 | 0 | 0 |
| CTONG2012564 | 0 | 7.180 | 0 | 0 | 0 | 0 | 0 |
| CTONG2012607 | 0 | 0 | 0 | 0 | 0 | 0 | 2.148 |
| CTONG2012661 | 0 | 0 | 0 | 0 | 0 | 0 | 26.030 |
| CTONG2012843 | 0 | 0 | 0 | 0 | 0 | 66.426 | 0 |
| CTONG2012879 | 0 | 12.359 | 0 | 24.588 | 0 | 0 | 2.112 |
| CTONG2012996 | 0 | 0 | 0 | 0 | 0 | 0 | 7.255 |
| CTONG2013352 | 0 | 49.384 | 0 | 0 | 0 | 0 | 0 |
| CTONG2013381 | 0 | 5.701 | 0 | 30.243 | 0 | 0 | 3.897 |
| CTONG2013907 | 0 | 0 | 0 | 10.392 | 0 | 0 | 0 |
| CTONG2013934 | 0 | 0 | 0 | 0 | 0 | 0 | 8.617 |
| CTONG2014280 | 0 | 0 | 0 | 30.295 | 0 | 0 | 0 |
| CTONG2014369 | 0 | 0 | 0 | 0 | 0 | 30.670 | 0 |
| CTONG2014898 | 0 | 0 | 0 | 0 | 0 | 0 | 14.331 |
| CTONG2015091 | 0 | 0 | 0 | 0 | 0 | 5.985 | 2.018 |
| CTONG2016056 | 0 | 0 | 57.220 | 0 | 0 | 0 | 0 |
| CTONG2016408 | 0 | 13.456 | 0 | 0 | 0 | 0 | 4.599 |
| CTONG2016575 | 1.802 | 7.228 | 0 | 1.198 | 0 | 0 | 9.265 |
| CTONG2016824 | 0 | 0 | 0 | 0 | 0 | 31.370 | 0 |
| CTONG2016931 | 0 | 0 | 0 | 0 | 0 | 22.490 | 0 |
| CTONG2017444 | 0 | 0 | 0 | 0 | 0 | 27.140 | 0 |
| CTONG2017500 | 0 | 0 | 0 | 0 | 0 | 0 | 3.291 |
| CTONG2018211 | 0 | 12.395 | 0 | 16.440 | 0 | 0 | 0 |
| CTONG2018383 | 0 | 0 | 0 | 10.895 | 0 | 0 | 0 |
| CTONG2018632 | 0 | 29.069 | 0 | 0 | 0 | 0 | 0 |
| CTONG2018637 | 0 | 0 | 0 | 0 | 0 | 0 | 16.300 |
| CTONG2018739 | 0 | 0 | 0 | 0 | 0 | 0 | 4.814 |
| CTONG2018808 | 0 | 0 | 0 | 0 | 0 | 58.046 | 0 |
| CTONG2018898 | 0 | 0.875 | 0 | 1.161 | 0 | 0 | 23.932 |
| CTONG2019400 | 0 | 0 | 0 | 0 | 0 | 0 | 24.523 |
| CTONG2019788 | 0 | 0 | 0 | 0 | 0 | 0 | 3.798 |
| CTONG2020411 | 0 | 25.022 | 0 | 16.593 | 0 | 0 | 0 |
| CTONG2020484 | 2.130 | 0 | 0 | 1.417 | 0 | 0 | 0 |
| CTONG2020560 | 0 | 0 | 0 | 3.616 | 0 | 0 | 0 |
| CTONG2020638 | 0 | 22.499 | 0 | 0 | 0 | 0 | 0 |
| CTONG2028124 | 0 | 0.913 | 0 | 1.211 | 0 | 3.704 | 1.249 |
| CTONG2028208 | 0 | 6.435 | 0 | 0 | 0 | 0 | 0 |
| CTONG3002835 | 0 | 1.996 | 0 | 0 | 0 | 0 | 2.730 |
| CTONG3003905 | 0 | 0 | 0 | 47.197 | 0 | 0 | 0 |
| CTONG3004726 | 0 | 0 | 0 | 0 | 0 | 0 | 21.039 |
| CTONG3006629 | 0 | 0 | 0 | 0 | 0 | 0 | 40.011 |
| CTONG3008894 | 2.634 | 9.247 | 0 | 7.008 | 0 | 0 | 12.642 |
| CTONG3009028 | 0 | 2.924 | 0 | 0 | 0 | 0 | 0 |
| CTONG3009239 | 0 | 2.624 | 0 | 0 | 0 | 0 | 0 |
| D30ST1000267 | 0 | 0 | 0 | 0 | 0 | 0 | 5.365 |
| D30ST1000270 | 0 | 4.695 | 0 | 6.227 | 0 | 0 | 0 |
| D30ST2002068 | 0 | 0.781 | 0 | 0 | 0 | 0 | 0 |
| D60ST2000464 | 0 | 0 | 0 | 0 | 0 | 0 | 1.361 |
| DFNES2000146 | 0 | 0 | 0 | 0 | 0 | 0 | 13.829 |
| DFNES2000153 | 0 | 0 | 0 | 0 | 0 | 0 | 3.977 |
| DFNES2000335 | 0 | 0 | 0 | 11.418 | 0 | 0 | 0 |
| DFNES2000443 | 0 | 0 | 0 | 3.272 | 0 | 0 | 0 |
| DFNES2000526 | 20.031 | 0 | 0 | 0 | 0 | 0 | 0 |
| DFNES2004346 | 11.342 | 0 | 0 | 0 | 0 | 0 | 0 |
| DFNES2004371 | 0 | 0 | 0 | 3.802 | 0 | 0 | 3.920 |
| DFNES2004608 | 0 | 0 | 0 | 0 | 0 | 55.567 | 0 |
| DFNES2005690 | 0 | 9.226 | 0 | 0 | 0 | 0 | 0 |
| DFNES2005766 | 0 | 0 | 52.757 | 0 | 0 | 0 | 0 |
| DFNES2006346 | 0 | 0 | 0 | 0 | 0 | 0 | 8.776 |
| DFNES2007634 | 0 | 1.615 | 0 | 0 | 0 | 0 | 2.208 |
| DFNES2008088 | 9.527 | 0 | 0 | 0 | 0 | 0 | 0 |
| DFNES2008160 | 0 | 0 | 0 | 0 | 0 | 0 | 11.131 |
| DFNES2009482 | 0 | 2.577 | 0 | 0 | 0 | 0 | 0 |
| DFNES2011499 | 0 | 22.548 | 0 | 0 | 0 | 0 | 0 |
| FCBBF1000024 | 0 | 0 | 0 | 10.583 | 0 | 0 | 10.910 |
| FCBBF1000027 | 0 | 1.997 | 0 | 3.531 | 0 | 0 | 1.820 |
| FCBBF1000038 | 0 | 0 | 0 | 0 | 0 | 0 | 41.870 |
| FCBBF1000053 | 19.616 | 0 | 0 | 0 | 0 | 0 | 3.362 |
| FCBBF1000061 | 0 | 0 | 0 | 0 | 0 | 0 | 17.530 |
| FCBBF1000063 | 0 | 0 | 0 | 14.415 | 0 | 0 | 7.430 |
| FCBBF1000077 | 0 | 1.472 | 0 | 0 | 0 | 0 | 13.079 |
| FCBBF1000115 | 0 | 0 | 0 | 0 | 0 | 0 | 25.252 |
| FCBBF1000118 | 0 | 0 | 0 | 0 | 0 | 0 | 56.514 |
| FCBBF1000139 | 0 | 0 | 0 | 12.640 | 0 | 0 | 34.747 |
| FCBBF1000197 | 0 | 1.941 | 0 | 0 | 0 | 0 | 0 |
| FCBBF1000280 | 0 | 10.265 | 0 | 0 | 0 | 0 | 0 |
| FCBBF1000294 | 0 | 0 | 0 | 0 | 0 | 7.935 | 6.688 |
| FCBBF1000305 | 0 | 0 | 0 | 0 | 0 | 0 | 66.858 |
| FCBBF1000377 | 0 | 19.652 | 0 | 0 | 0 | 0 | 13.434 |
| FCBBF1000403 | 0 | 15.241 | 0 | 0 | 0 | 0 | 0 |
| FCBBF1000412 | 0 | 0 | 0 | 0 | 0 | 0 | 7.212 |
| FCBBF1000449 | 0 | 0 | 0 | 0 | 0 | 0 | 16.611 |
| FCBBF1000466 | 0 | 2.315 | 0 | 1.535 | 0 | 0 | 3.165 |
| FCBBF1000476 | 0 | 0 | 0 | 0 | 0 | 0 | 10.789 |
| FCBBF1000509 | 6.762 | 0 | 0 | 0 | 0 | 0 | 0 |
| FCBBF1000556 | 0 | 0 | 0 | 5.138 | 0 | 0 | 0 |
| FCBBF1000563 | 5.443 | 24.565 | 0 | 7.240 | 0 | 0 | 1.866 |
| FCBBF1000574 | 0 | 0 | 0 | 0 | 0 | 0 | 15.473 |
| FCBBF1000671 | 0 | 0 | 0 | 0 | 0 | 0 | 51.023 |
| FCBBF1000675 | 0 | 23.076 | 0 | 0 | 0 | 0 | 0 |
| FCBBF1000687 | 31.307 | 0 | 0 | 0 | 0 | 0 | 0 |
| FCBBF1000732 | 0 | 5.089 | 0 | 0 | 0 | 0 | 0 |
| FCBBF1000760 | 0 | 1.190 | 0 | 0 | 0 | 0 | 0.814 |
| FCBBF2000038 | 0 | 0 | 0 | 0 | 0 | 0 | 5.006 |
| FCBBF2000199 | 0 | 0 | 0 | 0 | 0 | 0 | 31.279 |
| FCBBF2000259 | 0 | 0 | 0 | 0 | 0 | 0 | 14.857 |
| FCBBF2000362 | 30.336 | 0 | 0 | 0 | 0 | 0 | 0 |
| FCBBF2000566 | 0 | 0 | 0 | 0 | 0 | 0 | 32.322 |
| FCBBF2000591 | 0 | 0 | 0 | 0.691 | 0 | 0 | 0 |
| FCBBF2000885 | 5.563 | 0 | 0 | 7.399 | 0 | 0 | 9.534 |
| FCBBF2001001 | 0 | 17.368 | 0 | 0 | 0 | 0 | 0 |
| FCBBF2001105 | 0 | 16.549 | 0 | 0 | 0 | 0 | 3.771 |
| FCBBF2001183 | 0 | 0 | 0 | 0 | 0 | 0 | 8.217 |
| FCBBF2001211 | 0 | 11.646 | 0 | 0 | 0 | 0 | 0 |
| FCBBF2001291 | 6.895 | 3.457 | 0 | 4.585 | 0 | 0 | 0 |
| FCBBF2001309 | 0 | 7.906 | 0 | 0 | 0 | 0 | 21.620 |
| FCBBF2001538 | 0 | 2.769 | 0 | 0 | 0 | 0 | 0 |
| FCBBF2001594 | 0 | 6.911 | 0 | 0 | 0 | 0 | 9.449 |
| FCBBF2002349 | 0 | 7.174 | 0 | 0 | 0 | 0 | 4.904 |
| FCBBF2002801 | 0 | 0 | 0 | 0 | 0 | 0 | 12.897 |
| FCBBF2004066 | 0 | 0 | 0 | 0 | 0 | 0 | 5.009 |
| FCBBF2004373 | 0 | 0 | 0 | 56.608 | 0 | 0 | 0 |
| FCBBF2005733 | 0 | 0 | 0 | 0 | 0 | 0 | 40.207 |
| FCBBF2005909 | 0 | 0 | 0 | 0 | 0 | 0 | 31.013 |
| FCBBF2005966 | 34.323 | 0 | 0 | 0 | 0 | 0 | 0 |
| FCBBF2006781 | 0 | 0 | 0 | 30.502 | 0 | 0 | 0 |
| FCBBF2007302 | 0 | 0 | 0 | 56.608 | 0 | 0 | 0 |
| FCBBF2007556 | 0 | 0 | 0 | 0 | 0 | 0 | 11.353 |
| FCBBF2007840 | 45.139 | 0 | 0 | 0 | 0 | 0 | 0 |
| FCBBF3000102 | 0 | 0 | 0 | 0 | 0 | 0 | 77.076 |
| FCBBF3000110 | 0 | 0 | 0 | 0 | 0 | 0 | 8.023 |
| FCBBF3000115 | 0 | 0 | 0 | 0 | 0 | 0 | 9.916 |
| FCBBF3000168 | 0 | 14.795 | 0 | 0 | 0 | 0 | 20.229 |
| FCBBF3000388 | 8.952 | 0 | 0 | 5.953 | 0 | 0 | 6.137 |
| FCBBF3000434 | 6.709 | 0 | 0 | 0 | 0 | 0 | 2.300 |
| FCBBF3001081 | 0 | 0 | 0 | 6.328 | 0 | 0 | 0 |
| FCBBF3001281 | 0 | 0 | 0 | 49.073 | 0 | 0 | 0 |
| FCBBF3001377 | 0 | 0 | 0 | 24.817 | 0 | 0 | 0 |
| FCBBF3001632 | 5.595 | 2.805 | 0 | 7.441 | 0 | 0 | 13.424 |
| FCBBF3001657 | 0 | 0 | 0 | 0 | 0 | 0 | 11.722 |
| FCBBF3001818 | 0 | 16.553 | 0 | 0 | 0 | 0 | 0 |
| FCBBF3001855 | 0 | 5.089 | 0 | 0 | 0 | 20.639 | 3.479 |
| FCBBF3001890 | 9.969 | 4.999 | 0 | 0 | 0 | 0 | 3.417 |
| FCBBF3001977 | 0 | 13.536 | 0 | 17.952 | 0 | 0 | 9.253 |
| FCBBF3002556 | 0 | 0 | 0 | 0 | 0 | 0 | 40.207 |
| FCBBF3003787 | 0 | 0 | 0 | 0 | 0 | 72.131 | 0 |
| FCBBF3003800 | 1.428 | 2.863 | 0 | 1.899 | 0 | 0 | 10.766 |
| FCBBF3003902 | 0 | 0 | 0 | 56.608 | 0 | 0 | 0 |
| FCBBF3004261 | 0 | 0 | 0 | 21.802 | 0 | 0 | 0 |
| FCBBF3004323 | 0 | 0 | 0 | 0 | 0 | 0 | 27.378 |
| FCBBF3004409 | 0 | 0 | 0 | 0 | 0 | 0 | 40.207 |
| FCBBF3004473 | 0 | 4.176 | 0 | 0 | 0 | 0 | 2.854 |
| FCBBF3004715 | 0 | 0 | 0 | 0 | 0 | 0 | 4.652 |
| FCBBF3004955 | 0 | 0 | 0 | 0 | 0 | 0 | 28.723 |
| FCBBF3005320 | 0 | 0 | 0 | 0 | 0 | 0 | 6.778 |
| FCBBF3005330 | 2.511 | 3.777 | 0 | 0 | 0 | 0 | 2.582 |
| FCBBF3005497 | 0 | 0 | 0 | 3.789 | 0 | 0 | 2.930 |
| FCBBF3006821 | 0 | 10.388 | 0 | 0 | 0 | 0 | 0 |
| FCBBF3007859 | 0 | 11.805 | 0 | 0 | 0 | 0 | 8.070 |
| FCBBF3008100 | 0 | 0 | 0 | 0 | 0 | 0 | 18.533 |
| FCBBF3008382 | 0 | 0 | 0 | 0 | 0 | 0 | 13.381 |
| FCBBF3008644 | 0 | 0 | 0 | 0 | 0 | 0 | 10.704 |
| FCBBF3009069 | 0 | 0 | 0 | 0 | 0 | 0 | 5.563 |
| FCBBF3009152 | 0 | 0 | 0 | 45.841 | 0 | 0 | 0 |
| FCBBF3009479 | 35.849 | 0 | 0 | 0 | 0 | 0 | 0 |
| FCBBF3009541 | 0 | 0 | 0 | 5.961 | 0 | 0 | 1.536 |
| FCBBF3009703 | 0 | 0 | 0 | 0 | 0 | 0 | 40.207 |
| FCBBF3010012 | 0 | 0 | 0 | 0 | 0 | 0 | 22.837 |
| FCBBF3010142 | 0 | 0 | 0 | 0 | 0 | 0 | 17.930 |
| FCBBF3010695 | 0 | 0 | 0 | 0 | 0 | 0 | 3.081 |
| FCBBF3011485 | 0 | 0 | 0 | 0 | 0 | 0 | 49.834 |
| FCBBF3012347 | 0 | 3.989 | 0 | 0 | 0 | 0 | 0 |
| FCBBF3012443 | 0 | 49.588 | 0 | 0 | 0 | 0 | 0 |
| FCBBF3012667 | 0 | 0 | 0 | 0 | 0 | 0 | 30.319 |
| FCBBF3013589 | 0 | 0 | 0 | 19.996 | 0 | 0 | 0 |
| FCBBF3014260 | 0 | 0 | 0 | 21.767 | 0 | 0 | 8.415 |
| FCBBF3014770 | 0 | 0 | 0 | 0 | 0 | 0 | 11.854 |
| FCBBF3015100 | 0 | 0 | 0 | 0 | 0 | 0 | 43.390 |
| FCBBF3016134 | 0 | 0 | 0 | 25.089 | 0 | 0 | 0 |
| FCBBF3016451 | 0 | 0 | 0 | 56.608 | 0 | 0 | 0 |
| FCBBF3016667 | 0 | 0 | 0 | 0 | 0 | 0 | 15.903 |
| FCBBF3016928 | 0 | 1.592 | 25.214 | 2.112 | 0 | 0 | 1.089 |
| FCBBF3017024 | 0 | 0 | 0 | 0 | 0 | 0 | 12.734 |
| FCBBF3017288 | 0 | 0 | 0 | 0 | 0 | 0 | 40.207 |
| FCBBF3017396 | 0 | 0 | 0 | 0 | 0 | 0 | 7.608 |
| FCBBF3017918 | 0 | 23.796 | 0 | 31.560 | 0 | 0 | 0 |
| FCBBF3018453 | 16.741 | 8.394 | 0 | 0 | 0 | 0 | 22.954 |
| FCBBF3018826 | 0 | 0 | 0 | 23.511 | 0 | 0 | 0 |
| FCBBF3018949 | 0 | 0 | 0 | 0 | 0 | 0 | 17.580 |
| FCBBF3019437 | 0 | 0 | 0 | 0 | 0 | 0 | 40.207 |
| FCBBF3019455 | 0 | 0 | 0 | 0 | 0 | 0 | 20.235 |
| FCBBF3019714 | 0 | 0 | 0 | 0 | 0 | 0 | 2.545 |
| FCBBF3019784 | 0 | 49.588 | 0 | 0 | 0 | 0 | 0 |
| FCBBF3022504 | 0 | 0 | 0 | 0 | 0 | 0 | 19.361 |
| FCBBF3022593 | 0 | 12.591 | 0 | 0 | 0 | 0 | 8.607 |
| FCBBF3023368 | 0 | 0 | 0 | 0 | 0 | 0 | 6.096 |
| FCBBF3023667 | 0.481 | 2.655 | 3.821 | 1.600 | 0 | 0 | 0 |
| FCBBF3023887 | 0 | 0 | 0 | 0 | 0 | 0 | 58.866 |
| FCBBF3024002 | 0 | 0 | 0 | 0 | 0 | 0 | 28.201 |
| FCBBF3024623 | 0 | 5.091 | 0 | 0 | 0 | 0 | 0 |
| FCBBF3025528 | 0 | 0 | 0 | 0 | 0 | 0 | 11.995 |
| FCBBF3025730 | 0 | 0 | 0 | 0 | 0 | 0 | 24.596 |
| FCBBF3025737 | 0 | 0 | 0 | 15.666 | 0 | 0 | 0 |
| FCBBF3026048 | 0 | 0 | 0 | 0 | 0 | 0 | 40.207 |
| FCBBF3026651 | 0 | 0 | 0 | 0 | 0 | 0 | 9.065 |
| FCBBF3027854 | 0 | 28.447 | 0 | 0 | 0 | 0 | 0 |
| FCBBF3028244 | 3.365 | 1.687 | 0 | 8.951 | 0 | 0 | 11.534 |
| FCBBF3028593 | 0 | 0 | 0 | 0 | 0 | 0 | 20.063 |
| FCBBF4000017 | 0 | 0 | 0 | 0 | 0 | 0 | 11.034 |
| FCBBF4000142 | 0 | 0 | 0 | 8.811 | 0 | 0 | 18.167 |
| FCBBF4000193 | 0 | 0 | 0 | 0 | 0 | 0 | 40.207 |
| FCBBF4000446 | 8.166 | 6.825 | 0 | 5.431 | 0 | 0 | 17.729 |
| FCBBF5000061 | 0 | 38.068 | 0 | 0 | 0 | 0 | 0 |
| FCBBF5000165 | 11.929 | 0 | 0 | 0 | 0 | 0 | 8.178 |
| FEBRA1000008 | 0 | 42.162 | 0 | 0 | 0 | 0 | 0 |
| FEBRA1000030 | 59.247 | 0 | 0 | 0 | 0 | 0 | 0 |
| FEBRA1000057 | 0 | 9.464 | 0 | 0 | 0 | 0 | 0 |
| FEBRA1000088 | 15.466 | 0 | 0 | 0 | 0 | 0 | 0 |
| FEBRA1000188 | 0 | 0 | 0 | 5.044 | 0 | 0 | 36.398 |
| FEBRA2000105 | 0 | 0 | 0 | 1.729 | 0 | 0 | 0.891 |
| FEBRA2000228 | 0 | 0 | 0 | 0 | 0 | 0 | 9.814 |
| FEBRA2000253 | 0 | 0 | 0 | 0 | 0 | 0 | 36.369 |
| FEBRA2000297 | 28.740 | 0 | 0 | 0 | 0 | 0 | 9.852 |
| FEBRA2000377 | 0 | 0 | 0 | 0 | 0 | 0 | 12.427 |
| FEBRA2000397 | 42.133 | 0 | 0 | 0 | 0 | 0 | 28.885 |
| FEBRA2000452 | 0 | 3.645 | 0 | 9.669 | 0 | 0 | 11.213 |
| FEBRA2000462 | 0 | 0 | 0 | 0 | 0 | 0 | 7.013 |
| FEBRA2000532 | 0 | 0 | 0 | 0 | 0 | 0 | 3.973 |
| FEBRA2000581 | 2.883 | 0 | 0 | 0 | 0 | 0 | 12.847 |
| FEBRA2000733 | 1.832 | 0.306 | 0 | 0 | 0 | 0 | 0 |
| FEBRA2000740 | 0 | 2.055 | 0 | 0 | 0 | 0 | 4.215 |
| FEBRA2000757 | 0 | 0 | 0 | 0 | 0 | 0 | 19.021 |
| FEBRA2000787 | 0 | 0 | 0 | 4.760 | 0 | 0 | 7.361 |
| FEBRA2000793 | 0 | 0 | 0 | 0 | 0 | 0 | 28.583 |
| FEBRA2000815 | 0 | 0 | 0 | 0 | 0 | 0 | 17.964 |
| FEBRA2000881 | 0 | 0 | 0 | 7.629 | 0 | 0 | 0 |
| FEBRA2000972 | 0 | 0 | 0 | 0 | 0 | 0 | 18.817 |
| FEBRA2001146 | 0 | 0 | 0 | 0 | 0 | 0 | 13.892 |
| FEBRA2001197 | 0 | 0 | 0 | 0 | 0 | 0 | 35.024 |
| FEBRA2001227 | 0 | 0 | 0 | 0 | 0 | 0 | 4.593 |
| FEBRA2001245 | 9.141 | 0 | 0 | 0 | 0 | 0 | 12.534 |
| FEBRA2001492 | 0 | 0 | 0 | 15.601 | 0 | 0 | 4.021 |
| FEBRA2001571 | 0 | 0 | 0 | 0 | 0 | 15.455 | 7.816 |
| FEBRA2001581 | 14.860 | 0 | 0 | 0 | 0 | 0 | 0 |
| FEBRA2001669 | 0 | 0 | 0 | 2.582 | 0 | 0 | 23.955 |
| FEBRA2001715 | 0 | 0 | 0 | 7.210 | 0 | 0 | 9.910 |
| FEBRA2001745 | 4.542 | 0 | 0 | 0 | 0 | 0 | 9.341 |
| FEBRA2001814 | 9.589 | 0 | 0 | 0 | 0 | 0 | 3.287 |
| FEBRA2001828 | 0 | 9.887 | 0 | 0 | 0 | 0 | 0 |
| FEBRA2001867 | 0 | 0 | 0 | 17.865 | 0 | 0 | 4.604 |
| FEBRA2001990 | 0 | 0 | 0 | 0 | 0 | 0 | 4.508 |
| FEBRA2002027 | 0 | 0 | 0 | 0 | 0 | 0 | 3.147 |
| FEBRA2002352 | 12.580 | 6.308 | 0 | 0 | 0 | 0 | 8.625 |
| FEBRA2002410 | 0 | 0 | 0 | 0 | 0 | 0 | 33.259 |
| FEBRA2002456 | 0 | 0 | 0 | 2.629 | 0 | 0 | 8.130 |
| FEBRA2002525 | 0 | 0 | 0 | 0 | 0 | 0 | 33.259 |
| FEBRA2002682 | 0 | 0 | 0 | 0 | 0 | 0 | 33.259 |
| FEBRA2002707 | 0 | 0 | 0 | 0 | 0 | 26.556 | 0 |
| FEBRA2002781 | 0 | 0 | 0 | 0 | 0 | 0 | 25.500 |
| FEBRA2002792 | 0 | 2.432 | 0 | 3.226 | 0 | 0 | 0 |
| FEBRA2002931 | 0 | 0 | 0 | 15.825 | 0 | 0 | 12.236 |
| FEBRA2002933 | 0 | 1.345 | 0 | 2.676 | 0 | 0 | 0.460 |
| FEBRA2003100 | 2.387 | 2.394 | 0 | 0 | 0 | 0 | 5.727 |
| FEBRA2003128 | 0 | 15.858 | 0 | 0 | 0 | 0 | 7.227 |
| FEBRA2003198 | 0 | 0 | 0 | 0 | 0 | 0 | 33.259 |
| FEBRA2003470 | 0 | 0 | 0 | 0 | 0 | 0 | 18.131 |
| FEBRA2003541 | 0 | 0 | 0 | 0 | 0 | 0 | 32.225 |
| FEBRA2003750 | 0 | 0 | 0 | 0 | 0 | 0 | 2.268 |
| FEBRA2003833 | 0 | 0 | 0 | 16.670 | 0 | 0 | 4.296 |
| FEBRA2003926 | 0 | 1.269 | 0 | 5.047 | 0 | 5.145 | 0 |
| FEBRA2003930 | 0 | 3.266 | 0 | 0 | 0 | 0 | 2.233 |
| FEBRA2004042 | 0 | 0 | 0 | 15.337 | 0 | 0 | 5.270 |
| FEBRA2004110 | 0 | 0 | 0 | 0 | 0 | 0 | 33.259 |
| FEBRA2004191 | 0 | 0 | 0 | 0 | 0 | 0 | 24.636 |
| FEBRA2004224 | 0 | 0 | 0 | 0 | 0 | 0 | 1.438 |
| FEBRA2004293 | 0 | 0 | 0 | 0 | 0 | 0 | 6.605 |
| FEBRA2004325 | 0 | 6.401 | 0 | 0 | 0 | 0 | 0 |
| FEBRA2004412 | 0 | 0 | 0 | 0 | 0 | 0 | 3.647 |
| FEBRA2004485 | 0 | 0 | 0 | 0 | 0 | 0 | 17.243 |
| FEBRA2005114 | 24.318 | 12.194 | 0 | 0 | 0 | 0 | 0 |
| FEBRA2005361 | 0 | 0 | 0 | 0 | 0 | 0 | 1.740 |
| FEBRA2005416 | 0 | 0 | 0 | 2.588 | 0 | 0 | 1.334 |
| FEBRA2005476 | 0 | 4.733 | 0 | 6.277 | 0 | 0 | 0 |
| FEBRA2005572 | 6.457 | 0 | 0 | 0 | 0 | 0 | 0 |
| FEBRA2005734 | 6.120 | 3.069 | 0 | 4.070 | 0 | 0 | 0 |
| FEBRA2005778 | 0 | 0 | 0 | 7.229 | 0 | 0 | 11.179 |
| FEBRA2005824 | 0 | 0 | 0 | 0 | 0 | 5.498 | 1.853 |
| FEBRA2005995 | 0 | 2.548 | 0 | 0 | 0 | 0 | 19.157 |
| FEBRA2006092 | 2.253 | 0 | 0 | 1.499 | 0 | 0 | 1.545 |
| FEBRA2006112 | 0 | 0 | 0 | 0 | 0 | 0 | 8.200 |
| FEBRA2006255 | 0 | 0 | 0 | 0 | 0 | 0 | 14.569 |
| FEBRA2006270 | 0 | 0 | 0 | 0 | 0 | 8.616 | 1.452 |
| FEBRA2006357 | 0 | 0 | 0 | 0 | 0 | 9.763 | 4.114 |
| FEBRA2006368 | 0 | 0 | 0 | 0 | 0 | 0 | 11.580 |
| FEBRA2006372 | 0 | 0 | 38.646 | 3.237 | 0 | 0 | 3.337 |
| FEBRA2006396 | 0 | 14.606 | 0 | 0 | 0 | 0 | 29.955 |
| FEBRA2006401 | 0 | 1.824 | 0 | 0 | 82.760 | 0 | 1.247 |
| FEBRA2006485 | 0 | 0 | 0 | 0 | 0 | 0 | 3.779 |
| FEBRA2006521 | 0 | 0 | 0 | 0 | 0 | 0 | 9.501 |
| FEBRA2006553 | 0 | 0 | 0 | 0 | 0 | 0 | 4.915 |
| FEBRA2006627 | 0 | 0 | 0 | 0 | 0 | 0 | 25.634 |
| FEBRA2006808 | 0 | 0 | 0 | 0 | 0 | 0 | 5.099 |
| FEBRA2006873 | 0 | 8.160 | 0 | 0 | 0 | 0 | 11.157 |
| FEBRA2007458 | 29.545 | 14.815 | 0 | 0 | 0 | 0 | 20.255 |
| FEBRA2007544 | 0 | 0 | 0 | 0 | 0 | 0 | 9.124 |
| FEBRA2007622 | 0 | 1.875 | 0 | 0 | 0 | 7.603 | 2.563 |
| FEBRA2007714 | 0 | 0 | 0 | 0 | 0 | 0 | 31.096 |
| FEBRA2007793 | 0 | 0 | 0 | 0 | 0 | 0 | 49.917 |
| FEBRA2007801 | 0 | 0 | 0 | 5.950 | 0 | 0 | 0 |
| FEBRA2007880 | 0 | 0 | 0 | 0 | 0 | 0 | 4.676 |
| FEBRA2007900 | 0 | 0 | 0 | 18.681 | 0 | 0 | 0 |
| FEBRA2007901 | 0 | 0 | 0 | 0 | 0 | 0 | 4.604 |
| FEBRA2008036 | 0 | 0 | 0 | 0 | 0 | 0 | 10.646 |
| FEBRA2008081 | 2.566 | 5.147 | 0 | 6.827 | 0 | 10.438 | 4.399 |
| FEBRA2008087 | 1.949 | 0 | 5.158 | 0 | 0 | 0 | 0 |
| FEBRA2008201 | 0 | 0 | 0 | 17.339 | 0 | 0 | 0 |
| FEBRA2008210 | 0 | 0 | 0 | 0 | 0 | 0 | 8.332 |
| FEBRA2008287 | 0 | 0 | 0 | 0 | 0 | 0 | 3.662 |
| FEBRA2008302 | 0 | 3.868 | 0 | 0 | 0 | 0 | 2.644 |
| FEBRA2008360 | 0 | 0 | 0 | 0 | 0 | 0 | 25.401 |
| FEBRA2008583 | 0.479 | 0.721 | 0 | 0 | 43.582 | 0 | 10.016 |
| FEBRA2008662 | 11.505 | 0 | 0 | 0 | 0 | 0 | 1.972 |
| FEBRA2008861 | 0 | 10.618 | 0 | 4.694 | 0 | 0 | 14.517 |
| FEBRA2009022 | 12.103 | 0 | 0 | 0 | 0 | 0 | 0 |
| FEBRA2009419 | 0 | 4.802 | 0 | 0 | 0 | 0 | 6.566 |
| FEBRA2009514 | 0 | 6.019 | 0 | 0 | 0 | 0 | 0 |
| FEBRA2009588 | 0 | 0 | 0 | 0 | 0 | 0 | 42.136 |
| FEBRA2009846 | 0 | 0 | 0 | 0 | 0 | 0 | 25.645 |
| FEBRA2009986 | 0 | 0 | 0 | 0 | 0 | 0 | 23.954 |
| FEBRA2010719 | 0 | 0 | 0 | 0 | 0 | 0 | 21.556 |
| FEBRA2010746 | 0 | 0 | 0 | 0 | 0 | 0 | 63.946 |
| FEBRA2011090 | 0 | 0 | 0 | 0 | 0 | 0 | 21.561 |
| FEBRA2013699 | 0 | 13.005 | 0 | 0 | 0 | 0 | 8.891 |
| FEBRA2014122 | 0 | 0 | 0 | 0 | 0 | 0 | 33.259 |
| FEBRA2014417 | 1.957 | 0 | 0 | 0 | 0 | 0 | 2.012 |
| FEBRA2014578 | 0 | 0 | 0 | 0 | 0 | 0 | 27.189 |
| FEBRA2014939 | 0 | 0 | 0 | 0 | 0 | 0 | 51.886 |
| FEBRA2015085 | 5.526 | 0 | 0 | 7.349 | 0 | 0 | 22.729 |
| FEBRA2017024 | 0 | 0 | 0 | 0 | 0 | 0 | 11.986 |
| FEBRA2017850 | 0 | 0 | 0 | 0 | 0 | 0 | 22.253 |
| FEBRA2018203 | 0 | 0 | 0 | 0 | 0 | 0 | 18.850 |
| FEBRA2018407 | 0 | 29.440 | 0 | 0 | 0 | 0 | 0 |
| FEBRA2018476 | 0 | 0 | 0 | 0 | 0 | 0 | 2.715 |
| FEBRA2019242 | 0 | 58.974 | 0 | 0 | 0 | 0 | 0 |
| FEBRA2019637 | 59.247 | 0 | 0 | 0 | 0 | 0 | 0 |
| FEBRA2020886 | 0 | 0 | 0 | 0 | 0 | 0 | 15.061 |
| FEBRA2020977 | 0 | 0 | 0 | 0 | 0 | 0 | 6.546 |
| FEBRA2021966 | 0 | 0 | 0 | 0 | 0 | 31.791 | 0 |
| FEBRA2022055 | 0 | 0.967 | 0 | 0 | 0 | 0 | 0 |
| FEBRA2022504 | 5.952 | 2.985 | 0 | 3.958 | 0 | 0 | 0 |
| FEBRA2023377 | 0 | 0 | 0 | 0 | 0 | 0 | 9.637 |
| FEBRA2024019 | 4.860 | 0 | 0 | 3.232 | 0 | 0 | 0 |
| FEBRA2024727 | 0 | 0 | 0 | 0 | 0 | 0 | 33.259 |
| FEBRA2025427 | 0 | 0 | 0 | 0 | 0 | 0 | 3.079 |
| FEBRA2025838 | 2.977 | 0 | 0 | 0 | 0 | 0 | 1.020 |
| FEBRA2026582 | 0 | 42.162 | 0 | 0 | 0 | 0 | 0 |
| FEBRA2028503 | 0 | 0 | 0 | 0 | 0 | 0 | 8.685 |
| FEBRA2028516 | 0 | 6.236 | 0 | 11.027 | 0 | 0 | 2.842 |
| FELIV1000153 | 0 | 0 | 0 | 0 | 0 | 1.216 | 0.205 |
| HCASM1000115 | 0 | 1.884 | 0 | 2.498 | 0 | 0 | 1.288 |
| HCASM2000016 | 0 | 2.713 | 0 | 0 | 0 | 0 | 1.854 |
| HCASM2000138 | 0 | 7.675 | 0 | 0 | 0 | 0 | 0 |
| HCASM2000214 | 0 | 17.187 | 0 | 0 | 0 | 0 | 0 |
| HCASM2003018 | 0 | 0 | 0 | 0 | 0 | 0 | 10.964 |
| HCHON1000030 | 1.315 | 2.307 | 0 | 0.874 | 0 | 0 | 1.127 |
| HCHON1000131 | 0 | 7.456 | 0 | 0 | 0 | 0 | 5.097 |
| HCHON1000191 | 1.750 | 0.878 | 0 | 0 | 0 | 0 | 0 |
| HCHON2000028 | 0 | 5.766 | 0 | 0 | 0 | 0 | 0 |
| HCHON2000056 | 2.056 | 0 | 0 | 0 | 0 | 0 | 2.466 |
| HCHON2000160 | 3.648 | 0 | 0 | 0 | 0 | 0 | 2.501 |
| HCHON2000212 | 0 | 0 | 0 | 0 | 0 | 0 | 10.677 |
| HCHON2000226 | 1.461 | 0.733 | 0 | 0 | 0 | 0 | 0 |
| HCHON2000244 | 0 | 1.195 | 0 | 3.171 | 0 | 0 | 0 |
| HCHON2000265 | 0 | 0 | 58.221 | 0 | 0 | 0 | 0 |
| HCHON2000295 | 0 | 0.796 | 0 | 0 | 0 | 3.228 | 0 |
| HCHON2000323 | 0 | 0 | 0 | 0 | 0 | 0 | 4.602 |
| HCHON2000373 | 3.673 | 0 | 0 | 0 | 0 | 0 | 0 |
| HCHON2000473 | 0 | 0 | 0 | 0 | 0 | 0 | 7.272 |
| HCHON2000475 | 0 | 0 | 0 | 6.416 | 0 | 0 | 0 |
| HCHON2000508 | 0 | 0 | 0 | 0.900 | 0 | 2.752 | 0.464 |
| HCHON2000676 | 0 | 0 | 0 | 0 | 0 | 0 | 3.704 |
| HCHON2000698 | 10.388 | 0 | 0 | 0 | 0 | 0 | 3.561 |
| HCHON2000738 | 0 | 5.411 | 0 | 7.176 | 0 | 0 | 0 |
| HCHON2000743 | 0 | 0 | 0 | 0 | 0 | 0 | 7.585 |
| HCHON2000751 | 0 | 0 | 0 | 0 | 0 | 0 | 4.435 |
| HCHON2000815 | 20.242 | 5.075 | 0 | 0 | 0 | 0 | 3.469 |
| HCHON2000826 | 2.594 | 3.902 | 0 | 10.349 | 0 | 5.275 | 2.667 |
| HCHON2000832 | 12.822 | 19.287 | 0 | 17.053 | 0 | 0 | 0 |
| HCHON2001039 | 0 | 0 | 0 | 0 | 0 | 0 | 4.159 |
| HCHON2001084 | 0 | 4.173 | 0 | 0 | 0 | 0 | 0 |
| HCHON2001161 | 0 | 0 | 0 | 0 | 0 | 0 | 13.376 |
| HCHON2001200 | 3.135 | 0 | 0 | 3.127 | 0 | 3.188 | 1.612 |
| HCHON2001217 | 0 | 5.674 | 0 | 2.509 | 0 | 0 | 2.586 |
| HCHON2001359 | 0 | 1.547 | 0 | 0 | 0 | 0 | 0 |
| HCHON2001407 | 5.724 | 1.435 | 0 | 3.806 | 0 | 0 | 4.905 |
| HCHON2001548 | 0 | 0 | 0 | 27.583 | 0 | 0 | 0 |
| HCHON2001665 | 0 | 0 | 0 | 0 | 0 | 0 | 5.751 |
| HCHON2002247 | 10.174 | 0 | 0 | 6.766 | 0 | 0 | 0 |
| HCHON2004858 | 0 | 0 | 0 | 0 | 0 | 0 | 16.411 |
| HCHON2005921 | 0 | 0 | 0 | 0 | 0 | 0 | 5.152 |
| HCHON2006250 | 0 | 0 | 0 | 3.771 | 0 | 0 | 0 |
| HCHON2006459 | 0 | 19.614 | 0 | 0 | 0 | 0 | 0 |
| HCHON2007881 | 0 | 0 | 0 | 0 | 0 | 0 | 16.411 |
| HCHON2010543 | 0 | 0 | 0 | 0 | 0 | 0 | 11.144 |
| HEART1000074 | 0 | 0 | 0 | 1.830 | 0 | 0 | 2.829 |
| HEART1000118 | 0 | 0 | 0 | 0 | 0 | 0 | 13.769 |
| HEART1000132 | 0 | 0 | 0 | 0 | 0 | 21.405 | 0 |
| HEART1000142 | 0 | 0 | 0 | 0 | 0 | 13.139 | 0 |
| HEART1000149 | 0 | 1.707 | 0 | 0 | 0 | 0 | 1.167 |
| HEART1000173 | 9.512 | 0 | 0 | 0 | 0 | 0 | 0 |
| HEART1000185 | 0 | 0 | 0 | 0 | 0 | 11.653 | 0 |
| HEART2000411 | 0 | 0 | 0 | 0 | 0 | 15.549 | 0 |
| HEART2006521 | 0 | 4.346 | 0 | 5.764 | 0 | 0 | 0 |
| HEART2007231 | 0 | 17.605 | 0 | 11.675 | 0 | 0 | 0 |
| HEART2007696 | 0 | 0 | 0 | 12.004 | 0 | 0 | 0 |
| HHDPC1000083 | 0 | 1.779 | 0 | 0 | 0 | 0 | 2.432 |
| HHDPC1000114 | 5.267 | 2.641 | 0 | 0 | 0 | 0 | 0 |
| HHDPC1000118 | 15.191 | 3.809 | 0 | 10.102 | 0 | 0 | 0 |
| HHDPC1000163 | 4.557 | 0 | 0 | 3.031 | 0 | 0 | 0 |
| HHDPC2000055 | 0 | 0 | 0 | 4.407 | 0 | 0 | 0 |
| HHDPC2000102 | 0 | 8.628 | 0 | 0 | 0 | 0 | 5.899 |
| HHDPC2000104 | 0 | 0 | 0 | 4.459 | 0 | 0 | 4.596 |
| HHDPC2000455 | 0 | 0 | 0 | 1.324 | 0 | 0 | 0 |
| HHDPC2000456 | 0 | 0 | 0 | 3.742 | 0 | 0 | 1.929 |
| HHDPC2000462 | 0 | 20.514 | 0 | 0 | 0 | 0 | 0 |
| HHDPC2000572 | 0 | 11.897 | 0 | 0 | 0 | 0 | 0 |
| HHDPC2001896 | 33.980 | 0 | 0 | 0 | 0 | 0 | 0 |
| HHDPC2003439 | 0.922 | 1.387 | 0 | 0.613 | 0 | 0 | 5.371 |
| HHDPC2003983 | 0 | 6.072 | 0 | 3.356 | 0 | 0 | 1.038 |
| HHDPC2007267 | 0 | 0 | 0 | 1.177 | 0 | 7.196 | 0 |
| HHDPC2009528 | 0 | 0 | 0 | 0 | 0 | 0 | 8.074 |
| HLUNG1000037 | 0 | 0 | 0 | 21.178 | 0 | 0 | 0 |
| HLUNG1000055 | 1.962 | 0.984 | 0 | 2.609 | 0 | 0 | 4.035 |
| HLUNG1000091 | 0 | 0 | 0 | 0 | 0 | 0 | 18.384 |
| HLUNG2000412 | 0 | 4.866 | 0 | 0 | 0 | 0 | 3.326 |
| HLVNG2000846 | 0 | 0 | 0 | 0 | 0 | 0 | 5.612 |
| HLUNG2001146 | 0 | 0 | 0 | 0 | 0 | 0 | 13.024 |
| HLUNG2001518 | 0 | 0 | 0 | 0 | 0 | 0 | 4.075 |
| HLUNG2001633 | 0 | 0 | 0 | 0 | 0 | 0 | 9.947 |
| HLUNG2001677 | 0 | 6.385 | 0 | 0 | 0 | 0 | 0 |
| HLUNG2001712 | 0 | 0 | 0 | 0 | 0 | 0 | 4.096 |
| HLUNG2002334 | 33.313 | 0 | 0 | 0 | 0 | 0 | 0 |
| HLUNG2003042 | 0 | 4.326 | 0 | 0 | 0 | 17.544 | 0 |
| HLUNG2003306 | 0 | 0 | 0 | 0 | 0 | 0 | 15.907 |
| HLUNG2003378 | 0 | 16.824 | 0 | 0 | 0 | 0 | 0 |
| HLUNG2004067 | 0 | 0 | 0 | 26.538 | 0 | 0 | 0 |
| HLUNG2004159 | 0 | 3.295 | 0 | 0 | 0 | 0 | 2.252 |
| HLUNG2004170 | 0 | 0 | 0 | 0 | 0 | 0 | 7.655 |
| HLUNG2004388 | 0 | 8.114 | 0 | 0 | 0 | 0 | 0 |
| HLUNG2005133 | 0 | 0 | 0 | 34.145 | 0 | 0 | 0 |
| HLUNG2006812 | 0 | 0 | 0 | 7.127 | 0 | 21.793 | 3.673 |
| HLUNG2006843 | 0 | 18.861 | 0 | 25.015 | 0 | 0 | 0 |
| HLUNG2007210 | 0 | 0 | 0 | 9.908 | 0 | 0 | 0 |
| HLUNG2008153 | 24.669 | 0 | 0 | 0 | 0 | 0 | 0 |
| HLUNG2008348 | 0 | 0 | 0 | 0 | 0 | 0 | 31.571 |
| HLUNG2009225 | 0 | 0 | 0 | 0 | 0 | 0 | 3.747 |
| HLUNG2017262 | 0 | 0 | 0 | 0 | 0 | 0 | 9.850 |
| HSYRA1000062 | 3.105 | 2.335 | 0 | 1.032 | 0 | 0 | 3.193 |
| HSYRA1000119 | 0 | 1.671 | 0 | 0 | 0 | 0 | 0 |
| HSYRA1000137 | 0 | 6.772 | 0 | 0 | 0 | 0 | 18.519 |
| HSYRA1000148 | 0 | 0 | 0 | 0 | 0 | 0 | 8.192 |
| HSYRA1000152 | 0 | 2.167 | 0 | 2.875 | 0 | 0 | 7.409 |
| HSYRA1000178 | 0 | 4.557 | 0 | 0 | 0 | 0 | 3.115 |
| HSYRA2000135 | 14.334 | 7.187 | 0 | 0 | 0 | 0 | 0 |
| HSYRA2000137 | 0 | 4.119 | 0 | 0 | 0 | 0 | 0 |
| HSYRA2000159 | 0.437 | 1.534 | 0 | 0.291 | 0 | 0.889 | 1.947 |
| HSYRA2000190 | 8.607 | 0 | 0 | 0 | 0 | 0 | 2.950 |
| HSYRA2000221 | 0 | 0 | 0 | 0 | 0 | 0 | 11.760 |
| HSYRA2000224 | 0 | 0 | 0 | 0 | 0 | 0 | 2.234 |
| HSYRA2000232 | 8.039 | 1.344 | 0 | 1.782 | 0 | 0 | 6.430 |
| HSYRA2000248 | 2.463 | 0 | 0 | 1.638 | 0 | 0 | 4.222 |
| HSYRA2000255 | 0 | 0.815 | 0 | 0 | 0 | 0 | 0 |
| HSYRA2000332 | 0 | 0 | 0 | 0 | 0 | 0 | 1.938 |
| HSYRA2000605 | 0 | 10.873 | 0 | 0 | 0 | 0 | 0 |
| HSYRA2000743 | 0 | 4.219 | 0 | 5.595 | 0 | 0 | 5.768 |
| HSYRA2000828 | 0 | 2.589 | 0 | 10.301 | 0 | 0 | 0 |
| HSYRA2000925 | 0 | 0 | 0 | 0 | 0 | 0 | 0.275 |
| HSYRA2001105 | 0 | 0 | 0 | 6.640 | 0 | 0 | 0 |
| HSYRA2001138 | 0.924 | 1.390 | 0 | 0.614 | 0 | 0 | 0 |
| HSYRA2001225 | 0 | 0 | 0 | 3.563 | 0 | 0 | 1.837 |
| HSYRA2001396 | 0 | 2.761 | 0 | 0 | 0 | 0 | 0 |
| HSYRA2001476 | 0 | 0.877 | 0 | 1.164 | 0 | 0 | 0.600 |
| HSYRA2001567 | 0 | 0 | 0 | 0 | 0 | 0 | 2.784 |
| HSYRA2001574 | 0 | 1.367 | 0 | 1.813 | 0 | 0 | 1.869 |
| HSYRA2001595 | 8.258 | 1.035 | 16.393 | 5.492 | 0 | 0 | 1.415 |
| HSYRA2001615 | 0 | 11.306 | 0 | 0 | 0 | 0 | 2.576 |
| HSYRA2001621 | 0 | 0.667 | 5.280 | 0.442 | 0 | 0 | 0 |
| HSYRA2001638 | 0 | 0 | 0 | 10.902 | 0 | 0 | 5.619 |
| HSYRA2007650 | 0 | 8.644 | 0 | 0 | 0 | 0 | 0 |
| IMR321000158 | 0 | 5.533 | 0 | 0 | 0 | 0 | 0 |
| IMR321000207 | 50.668 | 0 | 0 | 0 | 0 | 0 | 0 |
| IMR321000210 | 3.673 | 5.526 | 0 | 4.886 | 0 | 7.470 | 3.778 |
| IMR321000219 | 1.017 | 4.590 | 0 | 2.029 | 0 | 2.069 | 4.881 |
| IMR321000242 | 0 | 0 | 0 | 0 | 0 | 0 | 6.986 |
| IMR321000266 | 0 | 5.143 | 0 | 0 | 0 | 0 | 0 |
| IMR322000072 | 0 | 0 | 0 | 1.934 | 0 | 0 | 0 |
| IMR322000121 | 0 | 0 | 0 | 0 | 0 | 8.216 | 1.385 |
| IMR322000127 | 0 | 0 | 0 | 3.733 | 0 | 0 | 1.924 |
| IMR322000144 | 0 | 0 | 0 | 3.931 | 0 | 0 | 2.026 |
| IMR322000223 | 2.467 | 2.475 | 0 | 0 | 0 | 0 | 0 |
| IMR322000243 | 0 | 3.118 | 0 | 0 | 0 | 6.323 | 2.132 |
| IMR322000302 | 21.500 | 10.781 | 0 | 0 | 0 | 0 | 0 |
| IMR322000316 | 9.023 | 5.278 | 0 | 1.000 | 0 | 0 | 10.310 |
| IMR322000555 | 0 | 0 | 0 | 0 | 0 | 0 | 7.411 |
| IMR322000730 | 0.497 | 0.249 | 3.944 | 0 | 0 | 0 | 0 |
| IMR322000775 | 0 | 0 | 0 | 0 | 0 | 30.557 | 0 |
| IMR322000791 | 18.803 | 0 | 0 | 0 | 0 | 0 | 0 |
| IMR322000917 | 0 | 0 | 0 | 0 | 0 | 0 | 14.674 |
| IMR322000935 | 5.426 | 0 | 0 | 0 | 0 | 0 | 1.860 |
| IMR322000973 | 0 | 0 | 0 | 1.943 | 0 | 0 | 0 |
| IMR322001317 | 5.724 | 0 | 0 | 0 | 0 | 0 | 0 |
| IMR322001332 | 9.649 | 0 | 0 | 0 | 0 | 0 | 0 |
| IMR322001380 | 7.184 | 3.602 | 0 | 0 | 0 | 0 | 0 |
| IMR322001435 | 3.405 | 0 | 0 | 0 | 0 | 0 | 1.167 |
| IMR322001541 | 6.131 | 0 | 0 | 0 | 0 | 0 | 0 |
| IMR322001600 | 0 | 0.585 | 0 | 1.164 | 0 | 2.373 | 0 |
| IMR322001670 | 0 | 0 | 0 | 0 | 0 | 0 | 5.667 |
| IMR322001710 | 0 | 3.524 | 0 | 4.674 | 0 | 14.294 | 0 |
| IMR322001879 | 0 | 35.028 | 0 | 0 | 0 | 0 | 0 |
| IMR322002993 | 0 | 0 | 0 | 0 | 0 | 0 | 19.337 |
| IMR322012529 | 0 | 0 | 0 | 0 | 0 | 0 | 17.550 |
| KIDNE1000143 | 0 | 1.208 | 0 | 0 | 0 | 0 | 0 |
| KIDNE1000145 | 0 | 0 | 0 | 6.665 | 0 | 0 | 0 |
| KIDNE2000085 | 0 | 0 | 0 | 4.957 | 0 | 0 | 0 |
| KIDNE2000252 | 6.195 | 1.553 | 0 | 0 | 0 | 0 | 6.370 |
| KIDNE2000330 | 0 | 0 | 0 | 8.228 | 0 | 0 | 0 |
| KIDNE2000403 | 0 | 0 | 0 | 1.991 | 0 | 0 | 0 |
| KIDNE2000574 | 0 | 0 | 0 | 0 | 0 | 0 | 0.930 |
| KIDNE2000665 | 7.016 | 0 | 0 | 0 | 0 | 0 | 2.405 |
| KIDNE2000721 | 0 | 0 | 0 | 0 | 0 | 0 | 26.272 |
| KIDNE2000947 | 0 | 0 | 0 | 0 | 0 | 0 | 9.419 |
| KIDNE2001162 | 3.383 | 0 | 0 | 1.125 | 0 | 0 | 0.580 |
| KIDNE2001467 | 0.945 | 2.368 | 0 | 2.513 | 0 | 1.921 | 4.857 |
| KIDNE2001979 | 0 | 0 | 0 | 28.399 | 0 | 0 | 0 |
| KIDNE2002191 | 0 | 0 | 0 | 34.358 | 0 | 0 | 17.710 |
| KIDNE2002252 | 0 | 3.961 | 0 | 0 | 0 | 0 | 2.708 |
| KIDNE2002265 | 0 | 0 | 0 | 4.383 | 0 | 0 | 11.297 |
| KIDNE2002883 | 0 | 0 | 0 | 0 | 0 | 16.715 | 0 |
| KIDNE2003305 | 0 | 0 | 0 | 2.029 | 0 | 0 | 0 |
| KIDNE2003941 | 0 | 1.833 | 0 | 3.647 | 0 | 0 | 0.627 |
| KIDNE2004475 | 9.329 | 0 | 0 | 0 | 0 | 0 | 9.593 |
| KIDNE2004534 | 0 | 0 | 0 | 8.996 | 0 | 0 | 9.274 |
| KIDNE2005042 | 0 | 0 | 0 | 0 | 0 | 0 | 10.002 |
| KIDNE2005321 | 35.810 | 17.956 | 0 | 0 | 0 | 0 | 0 |
| KIDNE2005676 | 0 | 7.670 | 0 | 6.781 | 0 | 0 | 1.748 |
| KIDNE2006014 | 0 | 10.792 | 0 | 7.156 | 0 | 0 | 0 |
| KIDNE2006030 | 0 | 3.878 | 0 | 0 | 0 | 0 | 5.302 |
| KIDNE2006248 | 2.254 | 3.390 | 0 | 0.749 | 0 | 0 | 0.386 |
| KIDNE2006465 | 0 | 5.623 | 0 | 7.458 | 0 | 0 | 19.220 |
| KIDNE2006775 | 0 | 3.329 | 0 | 0 | 0 | 0 | 0 |
| KIDNE2006811 | 0 | 0 | 0 | 0 | 0 | 0 | 4.043 |
| KIDNE2007328 | 0 | 6.431 | 0 | 0 | 0 | 0 | 0 |
| KIDNE2007352 | 1.507 | 1.511 | 0 | 0 | 0 | 3.064 | 1.033 |
| KIDNE2007954 | 15.279 | 0 | 0 | 0 | 0 | 0 | 0 |
| KIDNE2008048 | 1.944 | 6.824 | 0 | 1.293 | 0 | 3.954 | 5.998 |
| KIDNE2008914 | 0 | 0 | 0 | 0 | 0 | 0 | 21.609 |
| KIDNE2008987 | 10.440 | 0 | 0 | 0 | 0 | 0 | 0 |
| KIDNE2009019 | 0 | 0 | 0 | 0 | 0 | 0 | 26.272 |
| KIDNE2009332 | 0 | 34.265 | 0 | 0 | 0 | 0 | 0 |
| KIDNE2009367 | 0 | 0 | 0 | 0 | 0 | 5.555 | 0.936 |
| KIDNE2010430 | 0 | 17.330 | 0 | 0 | 0 | 0 | 0 |
| KIDNE2012025 | 0 | 0 | 67.526 | 0 | 0 | 0 | 0 |
| KIDNE2012188 | 0 | 16.225 | 0 | 21.519 | 0 | 0 | 0 |
| KIDNE2012316 | 0 | 0 | 0 | 40.874 | 0 | 0 | 0 |
| KIDNE2012440 | 0 | 0 | 0 | 0 | 0 | 0 | 2.199 |
| KIDNE2013845 | 0 | 0 | 0 | 0 | 0 | 0 | 5.554 |
| KIDNE2014290 | 0 | 0 | 0 | 0 | 0 | 0 | 0.871 |
| KIDNE2015445 | 0 | 0 | 0 | 0 | 0 | 0 | 4.268 |
| KIDNE2017040 | 0 | 0 | 0 | 0 | 0 | 0 | 8.355 |
| KIDNE2018217 | 21.992 | 11.027 | 0 | 0 | 0 | 0 | 0 |
| KIDNE2018891 | 0 | 0 | 0 | 0 | 0 | 0 | 7.576 |
| LIVER1000079 | 6.350 | 0 | 0 | 0 | 0 | 0 | 0 |
| LIVER2000033 | 0 | 2.514 | 0 | 0 | 0 | 0 | 0 |
| LIVER2001539 | 0 | 0 | 0 | 0 | 0 | 0 | 6.186 |
| LIVER2001608 | 0 | 0 | 0 | 0 | 0 | 0 | 1.631 |
| LIVER2006006 | 2.577 | 3.877 | 0 | 1.714 | 0 | 0 | 0 |
| LIVER2008465 | 0 | 0 | 0 | 15.950 | 0 | 0 | 0 |
| MAMGL1000032 | 0.584 | 0.293 | 0 | 0 | 0 | 0 | 0 |
| MAMGL1000035 | 0.730 | 0.732 | 0 | 0 | 0 | 0 | 0.500 |
| MAMGL1000173 | 0 | 0 | 0 | 0.639 | 0 | 0 | 0 |
| MESAN1000050 | 0 | 2.917 | 0 | 7.738 | 0 | 0 | 0 |
| MESAN1000101 | 0 | 0 | 0 | 0 | 0 | 0 | 14.341 |
| MESAN1000126 | 0 | 0 | 0 | 0 | 0 | 0 | 2.218 |
| MESAN1000147 | 0 | 37.266 | 0 | 24.712 | 0 | 0 | 0 |
| MESAN2000149 | 0 | 0 | 0 | 0 | 0 | 0 | 3.052 |
| MESAN2000267 | 4.452 | 0 | 0 | 1.974 | 0 | 3.018 | 2.035 |
| MESAN2000457 | 0 | 7.197 | 0 | 0 | 0 | 0 | 0 |
| MESAN2000501 | 0 | 5.452 | 0 | 0 | 0 | 0 | 0 |
| MESAN2000572 | 0 | 3.716 | 0 | 0 | 0 | 0 | 0 |
| MESAN2000608 | 2.850 | 0 | 0 | 1.895 | 0 | 5.795 | 0.977 |
| MESAN2001627 | 0 | 23.769 | 0 | 0 | 0 | 0 | 0 |
| MESAN2002086 | 0 | 3.026 | 0 | 1.003 | 0 | 0 | 0.517 |
| MESAN2002122 | 0 | 0 | 0 | 0 | 0 | 0 | 33.057 |
| MESAN2002186 | 7.944 | 0 | 0 | 0 | 0 | 0 | 0 |
| MESAN2002519 | 0 | 0 | 0 | 0 | 0 | 0 | 5.761 |
| MESAN2002844 | 26.260 | 0 | 0 | 0 | 0 | 0 | 0 |
| MESAN2002940 | 0 | 0 | 0 | 0 | 0 | 0 | 3.951 |
| MESAN2003035 | 0 | 0 | 0 | 0 | 0 | 0 | 8.903 |
| MESAN2003039 | 0 | 0 | 0 | 0 | 0 | 0 | 20.223 |
| MESAN2003058 | 0 | 0 | 0 | 14.434 | 0 | 0 | 7.440 |
| MESAN2003444 | 0 | 0 | 0 | 0 | 0 | 0 | 4.628 |
| MESAN2003622 | 2.178 | 2.184 | 0 | 0 | 0 | 4.429 | 0 |
| MESAN2005633 | 0 | 0 | 0 | 0 | 0 | 0 | 10.663 |
| MESAN2005689 | 0 | 0 | 0 | 7.477 | 0 | 0 | 7.708 |
| MESAN2005766 | 0 | 0 | 0 | 9.507 | 0 | 0 | 1.225 |
| MESAN2006043 | 0 | 14.474 | 0 | 0 | 0 | 0 | 0 |
| MESAN2006401 | 0 | 0 | 0 | 4.360 | 0 | 0 | 15.732 |
| MESAN2006563 | 0 | 2.510 | 0 | 1.664 | 0 | 0 | 2.573 |
| MESAN2006743 | 13.969 | 0 | 0 | 0 | 0 | 0 | 0 |
| MESAN2006953 | 0 | 12.461 | 0 | 0 | 0 | 0 | 0 |
| MESAN2008334 | 0 | 0 | 0 | 0 | 0 | 0 | 10.213 |
| MESAN2008926 | 0 | 0 | 0 | 0 | 0 | 0 | 3.966 |
| MESAN2008936 | 0 | 14.459 | 0 | 0 | 0 | 0 | 4.942 |
| MESAN2009019 | 0 | 0 | 0 | 56.483 | 0 | 0 | 0 |
| MESAN2011984 | 0 | 0 | 0 | 4.112 | 0 | 0 | 2.119 |
| MESAN2012054 | 0 | 4.636 | 0 | 0 | 0 | 0 | 3.169 |
| MESAN2012113 | 0 | 10.721 | 0 | 14.219 | 0 | 0 | 7.329 |
| MESAN2013284 | 8.902 | 6.696 | 0 | 8.880 | 0 | 0 | 9.154 |
| MESAN2013908 | 25.005 | 0 | 0 | 0 | 0 | 0 | 0 |
| MESAN2015391 | 0 | 0 | 0 | 0 | 0 | 0 | 25.095 |
| MESAN2015515 | 0 | 0 | 0 | 6.402 | 0 | 0 | 0 |
| MESAN2016552 | 0 | 0 | 0 | 0 | 0 | 0 | 30.229 |
| MESAN2017133 | 0 | 0 | 0 | 0 | 0 | 0 | 10.900 |
| NB9N41000011 | 0 | 0 | 0 | 0 | 0 | 0 | 2.644 |
| NB9N41000047 | 0 | 0 | 0 | 0 | 0 | 0 | 3.265 |
| NB9N41000121 | 0 | 0 | 0 | 0 | 0 | 0 | 0.933 |
| NB9N41000135 | 0 | 4.030 | 0 | 0 | 0 | 0 | 0 |
| NB9N41000142 | 0 | 0 | 0 | 6.452 | 0 | 0 | 0 |
| NB9N42000281 | 0 | 3.475 | 0 | 0 | 0 | 0 | 9.501 |
| NHNPC2000206 | 7.466 | 2.496 | 0 | 8.275 | 0 | 0 | 2.559 |
| NHNPC2000212 | 0 | 0 | 0 | 0 | 0 | 0 | 4.329 |
| NHNPC2001312 | 0 | 0 | 0 | 0 | 0 | 0 | 4.743 |
| NHNPC2001931 | 0 | 0 | 0 | 0 | 0 | 17.586 | 0 |
| NOVAR1000091 | 0 | 3.519 | 0 | 0 | 0 | 0 | 0 |
| NOVAR2000783 | 0 | 0 | 0 | 0 | 0 | 0 | 4.698 |
| NT2NE1000004 | 29.929 | 0 | 0 | 0 | 0 | 0 | 0 |
| NT2NE1000014 | 31.502 | 15.796 | 0 | 0 | 0 | 0 | 10.799 |
| NT2NE1000018 | 9.012 | 0 | 0 | 0 | 0 | 0 | 6.178 |
| NT2NE1000023 | 100 | 0 | 0 | 0 | 0 | 0 | 0 |
| NT2NE1000024 | 13.913 | 0 | 0 | 18.505 | 0 | 0 | 4.769 |
| NT2NE1000059 | 19.602 | 0 | 0 | 0 | 0 | 0 | 6.719 |
| NT2NE1000063 | 100 | 0 | 0 | 0 | 0 | 0 | 0 |
| NT2NE1000073 | 57.716 | 14.470 | 0 | 0 | 0 | 0 | 0 |
| NT2NE1000083 | 100 | 0 | 0 | 0 | 0 | 0 | 0 |
| NT2NE1000120 | 100 | 0 | 0 | 0 | 0 | 0 | 0 |
| NT2NE1000122 | 81.295 | 0 | 0 | 0 | 0 | 0 | 0 |
| NT2NE1000163 | 1.468 | 2.208 | 0 | 0 | 0 | 0 | 0.503 |
| NT2NE1000175 | 100 | 0 | 0 | 0 | 0 | 0 | 0 |
| NT2NE1000181 | 66.155 | 0 | 0 | 0 | 0 | 0 | 0 |
| NT2NE1000185 | 100 | 0 | 0 | 0 | 0 | 0 | 0 |
| NT2NE2000038 | 100 | 0 | 0 | 0 | 0 | 0 | 0 |
| NT2NE2000054 | 100 | 0 | 0 | 0 | 0 | 0 | 0 |
| NT2NE2000056 | 1.424 | 1.428 | 0 | 0 | 0 | 2.895 | 3.416 |
| NT2NE2000057 | 100 | 0 | 0 | 0 | 0 | 0 | 0 |
| NT2NE2000064 | 100 | 0 | 0 | 0 | 0 | 0 | 0 |
| NT2NE2000109 | 100 | 0 | 0 | 0 | 0 | 0 | 0 |
| NT2NE2000137 | 100 | 0 | 0 | 0 | 0 | 0 | 0 |
| NT2NE2000156 | 46.158 | 23.145 | 0 | 30.696 | 0 | 0 | 0 |
| NT2NE2000174 | 4.149 | 0 | 0 | 1.839 | 0 | 0 | 0 |
| NT2NE2000195 | 11.142 | 0 | 0 | 0 | 0 | 0 | 0 |
| NT2NE2000222 | 100 | 0 | 0 | 0 | 0 | 0 | 0 |
| NT2NE2000259 | 30.583 | 0 | 0 | 0 | 0 | 0 | 6.989 |
| NT2NE2000260 | 0.438 | 0 | 0 | 0.291 | 0 | 0 | 0 |
| NT2NE2000284 | 100 | 0 | 0 | 0 | 0 | 0 | 0 |
| NT2NE2000299 | 100 | 0 | 0 | 0 | 0 | 0 | 0 |
| NT2NE2000327 | 9.117 | 0 | 0 | 18.189 | 0 | 0 | 0 |
| NT2NE2000353 | 66.235 | 0 | 0 | 0 | 0 | 0 | 0 |
| NT2NE2000356 | 81.295 | 0 | 0 | 0 | 0 | 0 | 0 |
| NT2NE2000369 | 100 | 0 | 0 | 0 | 0 | 0 | 0 |
| NT2NE2000374 | 100 | 0 | 0 | 0 | 0 | 0 | 0 |
| NT2NE2000383 | 5.507 | 0 | 0 | 0 | 0 | 0 | 1.888 |
| NT2NE2000384 | 8.341 | 0 | 0 | 0 | 0 | 0 | 0 |
| NT2NE2000386 | 56.001 | 0 | 0 | 0 | 0 | 0 | 0 |
| NT2NE2000390 | 100 | 0 | 0 | 0 | 0 | 0 | 0 |
| NT2NE2000392 | 6.471 | 0 | 0 | 0 | 0 | 0 | 14.418 |
| NT2NE2000455 | 75.239 | 0 | 0 | 0 | 0 | 0 | 0 |
| NT2NE2000470 | 19.777 | 0 | 0 | 0 | 0 | 0 | 13.558 |
| NT2NE2000488 | 100 | 0 | 0 | 0 | 0 | 0 | 0 |
| NT2NE2000517 | 51.465 | 0 | 0 | 34.226 | 0 | 0 | 0 |
| NT2NE2000536 | 31.191 | 0 | 0 | 0 | 0 | 0 | 0 |
| NT2NE2000546 | 100 | 0 | 0 | 0 | 0 | 0 | 0 |
| NT2NE2000548 | 76.570 | 0 | 0 | 0 | 0 | 0 | 0 |
| NT2NE2000550 | 100 | 0 | 0 | 0 | 0 | 0 | 0 |
| NT2NE2000575 | 1.742 | 0.874 | 0 | 0 | 0 | 0 | 0.299 |
| NT2NE2000586 | 100 | 0 | 0 | 0 | 0 | 0 | 0 |
| NT2NE2000612 | 100 | 0 | 0 | 0 | 0 | 0 | 0 |
| NT2NE2000636 | 26.172 | 6.562 | 0 | 0 | 0 | 0 | 0 |
| NT2NE2000658 | 100 | 0 | 0 | 0 | 0 | 0 | 0 |
| NT2NE2000706 | 100 | 0 | 0 | 0 | 0 | 0 | 0 |
| NT2NE2000707 | 2.426 | 2.433 | 0 | 3.226 | 0 | 0 | 4.157 |
| NT2NE2000763 | 4.349 | 0 | 0 | 2.892 | 0 | 0 | 4.473 |
| NT2NE2000802 | 100 | 0 | 0 | 0 | 0 | 0 | 0 |
| NT2NE2000808 | 100 | 0 | 0 | 0 | 0 | 0 | 0 |
| NT2NE2000809 | 12.284 | 0 | 0 | 16.339 | 0 | 0 | 0 |
| NT2NE2000864 | 83.623 | 0 | 0 | 0 | 0 | 0 | 0 |
| NT2NE2000909 | 23.727 | 0 | 0 | 0 | 0 | 0 | 12.200 |
| NT2NE2000913 | 26.733 | 0 | 0 | 17.778 | 0 | 0 | 9.163 |
| NT2NE2000950 | 18.995 | 9.524 | 0 | 0 | 0 | 0 | 0 |
| NT2NE2000951 | 100 | 0 | 0 | 0 | 0 | 0 | 0 |
| NT2NE2000963 | 4.157 | 1.251 | 0 | 0.553 | 0 | 0 | 0 |
| NT2NE2000980 | 55.327 | 27.743 | 0 | 0 | 0 | 0 | 0 |
| NT2NE2001000 | 3.119 | 1.564 | 0 | 0 | 0 | 0 | 1.069 |
| NT2NE2001005 | 84.719 | 0 | 0 | 0 | 0 | 0 | 0 |
| NT2NE2001021 | 100 | 0 | 0 | 0 | 0 | 0 | 0 |
| NT2NE2001040 | 56.184 | 0 | 0 | 0 | 0 | 0 | 0 |
| NT2NE2001048 | 5.424 | 2.720 | 0 | 3.607 | 0 | 11.031 | 0 |
| NT2NE2001049 | 31.847 | 0 | 0 | 21.179 | 0 | 0 | 0 |
| NT2NE2001142 | 100 | 0 | 0 | 0 | 0 | 0 | 0 |
| NT2NE2001156 | 20.368 | 0 | 0 | 0 | 0 | 0 | 0 |
| NT2NE2001176 | 8.279 | 2.076 | 0 | 0 | 0 | 0 | 7.095 |
| NT2NE2001180 | 50.569 | 0 | 0 | 0 | 0 | 0 | 0 |
| NT2NE2001181 | 67.567 | 0 | 0 | 0 | 0 | 0 | 0 |
| NT2NE2001188 | 100 | 0 | 0 | 0 | 0 | 0 | 0 |
| NT2NE2001191 | 100 | 0 | 0 | 0 | 0 | 0 | 0 |
| NT2NE2001247 | 100 | 0 | 0 | 0 | 0 | 0 | 0 |
| NT2NE2001252 | 100 | 0 | 0 | 0 | 0 | 0 | 0 |
| NT2NE2001324 | 83.623 | 0 | 0 | 0 | 0 | 0 | 0 |
| NT2NE2001326 | 76.390 | 0 | 0 | 0 | 0 | 0 | 0 |
| NT2NE2001337 | 16.469 | 0 | 0 | 10.952 | 0 | 0 | 5.645 |
| NT2NE2001364 | 38.745 | 0 | 0 | 0 | 0 | 0 | 0 |
| NT2NE2001372 | 100 | 0 | 0 | 0 | 0 | 0 | 0 |
| NT2NE2001397 | 47.665 | 0 | 0 | 0 | 0 | 0 | 0 |
| NT2NE2001403 | 37.604 | 0 | 0 | 12.504 | 0 | 0 | 6.445 |
| NT2NE2001428 | 100 | 0 | 0 | 0 | 0 | 0 | 0 |
| NT2NE2001432 | 100 | 0 | 0 | 0 | 0 | 0 | 0 |
| NT2NE2001435 | 3.853 | 0 | 0 | 0 | 0 | 0 | 14.528 |
| NT2NE2001442 | 18.110 | 0 | 0 | 0 | 0 | 0 | 6.208 |
| NT2NE2001524 | 100 | 0 | 0 | 0 | 0 | 0 | 0 |
| NT2NE2001530 | 25.357 | 0 | 0 | 0 | 0 | 0 | 0 |
| NT2NE2001545 | 19.481 | 0 | 0 | 8.637 | 0 | 0 | 2.226 |
| NT2NE2001598 | 45.971 | 0 | 0 | 0 | 0 | 0 | 0 |
| NT2NE2001617 | 16.442 | 0 | 0 | 0 | 0 | 0 | 0 |
| NT2NE2001623 | 100 | 0 | 0 | 0 | 0 | 0 | 0 |
| NT2NE2001626 | 100 | 0 | 0 | 0 | 0 | 0 | 0 |
| NT2NE2001634 | 100 | 0 | 0 | 0 | 0 | 0 | 0 |
| NT2NE2001648 | 31.454 | 0 | 0 | 0 | 0 | 0 | 0 |
| NT2NE2001655 | 20.296 | 2.544 | 0 | 0 | 0 | 0 | 0 |
| NT2NE2001660 | 28.385 | 0 | 0 | 0 | 0 | 0 | 0 |
| NT2NE2001666 | 100 | 0 | 0 | 0 | 0 | 0 | 0 |
| NT2NE2001677 | 100 | 0 | 0 | 0 | 0 | 0 | 0 |
| NT2NE2001697 | 19.787 | 0 | 0 | 0 | 0 | 0 | 0 |
| NT2NE2001698 | 100 | 0 | 0 | 0 | 0 | 0 | 0 |
| NT2NE2001723 | 100 | 0 | 0 | 0 | 0 | 0 | 0 |
| NT2NE2001725 | 100 | 0 | 0 | 0 | 0 | 0 | 0 |
| NT2NE2001793 | 7.518 | 15.079 | 0 | 5.000 | 0 | 0 | 5.154 |
| NT2NE2001874 | 100 | 0 | 0 | 0 | 0 | 0 | 0 |
| NT2NE2001889 | 100 | 0 | 0 | 0 | 0 | 0 | 0 |
| NT2NE2001923 | 100 | 0 | 0 | 0 | 0 | 0 | 0 |
| NT2NE2002100 | 81.295 | 0 | 0 | 0 | 0 | 0 | 0 |
| NT2NE2002162 | 44.305 | 0 | 0 | 0 | 0 | 0 | 0 |
| NT2NE2002186 | 100 | 0 | 0 | 0 | 0 | 0 | 0 |
| NT2NE2002285 | 100 | 0 | 0 | 0 | 0 | 0 | 0 |
| NT2NE2002311 | 100 | 0 | 0 | 0 | 0 | 0 | 0 |
| NT2NE2002620 | 3.466 | 0 | 0 | 0 | 0 | 0 | 0 |
| NT2NE2002651 | 100 | 0 | 0 | 0 | 0 | 0 | 0 |
| NT2NE2002732 | 100 | 0 | 0 | 0 | 0 | 0 | 0 |
| NT2NE2002768 | 100 | 0 | 0 | 0 | 0 | 0 | 0 |
| NT2NE2002791 | 100 | 0 | 0 | 0 | 0 | 0 | 0 |
| NT2NE2002856 | 100 | 0 | 0 | 0 | 0 | 0 | 0 |
| NT2NE2002870 | 8.273 | 0 | 0 | 0 | 0 | 0 | 2.836 |
| NT2NE2003150 | 100 | 0 | 0 | 0 | 0 | 0 | 0 |
| NT2NE2003185 | 27.476 | 0 | 0 | 0 | 0 | 0 | 9.418 |
| NT2NE2003252 | 100 | 0 | 0 | 0 | 0 | 0 | 0 |
| NT2NE2003280 | 26.374 | 0 | 0 | 0 | 0 | 0 | 27.122 |
| NT2NE2003308 | 100 | 0 | 0 | 0 | 0 | 0 | 0 |
| NT2NE2003309 | 100 | 0 | 0 | 0 | 0 | 0 | 0 |
| NT2NE2003315 | 100 | 0 | 0 | 0 | 0 | 0 | 0 |
| NT2NE2003393 | 4.658 | 0 | 0 | 0 | 0 | 0 | 0 |
| NT2NE2003408 | 100 | 0 | 0 | 0 | 0 | 0 | 0 |
| NT2NE2003457 | 100 | 0 | 0 | 0 | 0 | 0 | 0 |
| NT2NE2003485 | 8.436 | 0 | 0 | 0 | 0 | 0 | 2.892 |
| NT2NE2003540 | 100 | 0 | 0 | 0 | 0 | 0 | 0 |
| NT2NE2003569 | 6.289 | 6.307 | 0 | 0 | 0 | 12.790 | 0 |
| NT2NE2003571 | 100 | 0 | 0 | 0 | 0 | 0 | 0 |
| NT2NE2003705 | 100 | 0 | 0 | 0 | 0 | 0 | 0 |
| NT2NE2003752 | 100 | 0 | 0 | 0 | 0 | 0 | 0 |
| NT2NE2003887 | 22.619 | 0 | 0 | 0 | 0 | 0 | 7.753 |
| NT2NE2003921 | 44.218 | 0 | 0 | 0 | 0 | 0 | 0 |
| NT2NE2004255 | 21.586 | 0 | 0 | 0 | 0 | 0 | 7.399 |
| NT2NE2004378 | 100 | 0 | 0 | 0 | 0 | 0 | 0 |
| NT2NE2004490 | 100 | 0 | 0 | 0 | 0 | 0 | 0 |
| NT2NE2004519 | 12.988 | 3.256 | 0 | 0 | 0 | 0 | 2.226 |
| NT2NE2004606 | 100 | 0 | 0 | 0 | 0 | 0 | 0 |
| NT2NE2004716 | 6.956 | 0 | 0 | 0 | 0 | 0 | 0 |
| NT2NE2004740 | 76.390 | 0 | 0 | 0 | 0 | 0 | 0 |
| NT2NE2004787 | 100 | 0 | 0 | 0 | 0 | 0 | 0 |
| NT2NE2004916 | 100 | 0 | 0 | 0 | 0 | 0 | 0 |
| NT2NE2005035 | 100 | 0 | 0 | 0 | 0 | 0 | 0 |
| NT2NE2005223 | 100 | 0 | 0 | 0 | 0 | 0 | 0 |
| NT2NE2005276 | 100 | 0 | 0 | 0 | 0 | 0 | 0 |
| NT2NE2005317 | 46.084 | 0 | 0 | 30.647 | 0 | 0 | 0 |
| NT2NE2005323 | 81.295 | 0 | 0 | 0 | 0 | 0 | 0 |
| NT2NE2005358 | 75.239 | 0 | 0 | 0 | 0 | 0 | 0 |
| NT2NE2005371 | 19.983 | 0 | 0 | 0 | 0 | 0 | 0 |
| NT2NE2005395 | 49.516 | 0 | 0 | 0 | 0 | 0 | 0 |
| NT2NE2005419 | 41.962 | 0 | 0 | 0 | 0 | 0 | 14.384 |
| NT2NE2005441 | 11.259 | 0 | 0 | 0 | 0 | 0 | 0 |
| NT2NE2005517 | 100 | 0 | 0 | 0 | 0 | 0 | 0 |
| NT2NE2005537 | 100 | 0 | 0 | 0 | 0 | 0 | 0 |
| NT2NE2005555 | 100 | 0 | 0 | 0 | 0 | 0 | 0 |
| NT2NE2005588 | 61.235 | 0 | 0 | 0 | 0 | 0 | 0 |
| NT2NE2005676 | 100 | 0 | 0 | 0 | 0 | 0 | 0 |
| NT2NE2005688 | 2.706 | 0 | 0 | 1.800 | 0 | 0 | 0 |
| NT2NE2005720 | 34.046 | 0 | 0 | 0 | 0 | 0 | 0 |
| NT2NE2005784 | 100 | 0 | 0 | 0 | 0 | 0 | 0 |
| NT2NE2005804 | 100 | 0 | 0 | 0 | 0 | 0 | 0 |
| NT2NE2005821 | 100 | 0 | 0 | 0 | 0 | 0 | 0 |
| NT2NE2005876 | 10.692 | 0 | 0 | 0 | 0 | 10.871 | 12.827 |
| NT2NE2005890 | 64.265 | 0 | 0 | 0 | 0 | 0 | 0 |
| NT2NE2005921 | 100 | 0 | 0 | 0 | 0 | 0 | 0 |
| NT2NE2005968 | 100 | 0 | 0 | 0 | 0 | 0 | 0 |
| NT2NE2006075 | 28.548 | 0 | 0 | 0 | 0 | 0 | 0 |
| NT2NE2006087 | 36.785 | 0 | 0 | 0 | 0 | 0 | 12.609 |
| NT2NE2006103 | 31.379 | 0 | 0 | 20.868 | 0 | 0 | 0 |
| NT2NE2006217 | 100 | 0 | 0 | 0 | 0 | 0 | 0 |
| NT2NE2006288 | 100 | 0 | 0 | 0 | 0 | 0 | 0 |
| NT2NE2006382 | 100 | 0 | 0 | 0 | 0 | 0 | 0 |
| NT2NE2006387 | 100 | 0 | 0 | 0 | 0 | 0 | 0 |
| NT2NE2006427 | 66.603 | 33.397 | 0 | 0 | 0 | 0 | 0 |
| NT2NE2006432 | 100 | 0 | 0 | 0 | 0 | 0 | 0 |
| NT2NE2006458 | 5.123 | 0 | 0 | 0 | 0 | 0 | 0 |
| NT2NE2006478 | 38.940 | 0 | 0 | 0 | 0 | 0 | 0 |
| NT2NE2006531 | 100 | 0 | 0 | 0 | 0 | 0 | 0 |
| NT2NE2006659 | 65.576 | 0 | 0 | 0 | 0 | 0 | 0 |
| NT2NE2006784 | 100 | 0 | 0 | 0 | 0 | 0 | 0 |
| NT2NE2006813 | 100 | 0 | 0 | 0 | 0 | 0 | 0 |
| NT2NE2006894 | 100 | 0 | 0 | 0 | 0 | 0 | 0 |
| NT2NE2006909 | 1.034 | 0 | 0 | 0 | 94.074 | 0 | 0 |
| NT2NE2006942 | 100 | 0 | 0 | 0 | 0 | 0 | 0 |
| NT2NE2006958 | 59.247 | 0 | 0 | 0 | 0 | 0 | 0 |
| NT2NE2007052 | 100 | 0 | 0 | 0 | 0 | 0 | 0 |
| NT2NE2007220 | 100 | 0 | 0 | 0 | 0 | 0 | 0 |
| NT2NE2007425 | 100 | 0 | 0 | 0 | 0 | 0 | 0 |
| NT2NE2007725 | 100 | 0 | 0 | 0 | 0 | 0 | 0 |
| NT2NE2007727 | 22.173 | 77.827 | 0 | 0 | 0 | 0 | 0 |
| NT2NE2007786 | 14.439 | 0 | 0 | 0 | 0 | 0 | 0 |
| NT2NE2007877 | 100 | 0 | 0 | 0 | 0 | 0 | 0 |
| NT2NE2007967 | 100 | 0 | 0 | 0 | 0 | 0 | 0 |
| NT2NE2008017 | 100 | 0 | 0 | 0 | 0 | 0 | 0 |
| NT2NE2008060 | 74.472 | 0 | 0 | 0 | 0 | 0 | 25.528 |
| NT2NE2008077 | 35.311 | 0 | 0 | 0 | 0 | 0 | 0 |
| NT2NE2008213 | 30.158 | 0 | 0 | 0 | 0 | 0 | 0 |
| NT2NE2008260 | 100 | 0 | 0 | 0 | 0 | 0 | 0 |
| NT2NE2008378 | 100 | 0 | 0 | 0 | 0 | 0 | 0 |
| NT2NE2008607 | 100 | 0 | 0 | 0 | 0 | 0 | 0 |
| NT2NE2008639 | 100 | 0 | 0 | 0 | 0 | 0 | 0 |
| NT2NE2008727 | 100 | 0 | 0 | 0 | 0 | 0 | 0 |
| NT2NE2008783 | 100 | 0 | 0 | 0 | 0 | 0 | 0 |
| NT2NE2008785 | 34.001 | 0 | 0 | 0 | 0 | 0 | 0 |
| NT2NE2008803 | 83.623 | 0 | 0 | 0 | 0 | 0 | 0 |
| NT2NE2008867 | 100 | 0 | 0 | 0 | 0 | 0 | 0 |
| NT2NE2008961 | 100 | 0 | 0 | 0 | 0 | 0 | 0 |
| NT2NE2008997 | 100 | 0 | 0 | 0 | 0 | 0 | 0 |
| NT2NE2009138 | 100 | 0 | 0 | 0 | 0 | 0 | 0 |
| NT2NE2009295 | 7.230 | 0 | 57.410 | 4.808 | 0 | 0 | 0 |
| NT2NE2009426 | 100 | 0 | 0 | 0 | 0 | 0 | 0 |
| NT2NE2009523 | 56.323 | 0 | 0 | 0 | 0 | 0 | 0 |
| NT2NE2009608 | 100 | 0 | 0 | 0 | 0 | 0 | 0 |
| NT2NE2009691 | 100 | 0 | 0 | 0 | 0 | 0 | 0 |
| NT2NE2010056 | 29.041 | 0 | 0 | 0 | 0 | 0 | 0 |
| NT2NE2010105 | 100 | 0 | 0 | 0 | 0 | 0 | 0 |
| NT2NE2010150 | 100 | 0 | 0 | 0 | 0 | 0 | 0 |
| NT2NE2010400 | 100 | 0 | 0 | 0 | 0 | 0 | 0 |
| NT2NE2010632 | 100 | 0 | 0 | 0 | 0 | 0 | 0 |
| NT2NE2010633 | 100 | 0 | 0 | 0 | 0 | 0 | 0 |
| NT2NE2010654 | 100 | 0 | 0 | 0 | 0 | 0 | 0 |
| NT2NE2010688 | 78.374 | 0 | 0 | 0 | 0 | 0 | 0 |
| NT2NE2010781 | 100 | 0 | 0 | 0 | 0 | 0 | 0 |
| NT2NE2010842 | 3.417 | 5.140 | 0 | 6.817 | 0 | 0 | 2.343 |
| NT2NE2010854 | 84.719 | 0 | 0 | 0 | 0 | 0 | 0 |
| NT2NE2011033 | 66.235 | 0 | 0 | 0 | 0 | 0 | 0 |
| NT2NE2011036 | 100 | 0 | 0 | 0 | 0 | 0 | 0 |
| NT2NE2011107 | 43.585 | 0 | 0 | 0 | 0 | 0 | 14.940 |
| NT2NE2011119 | 33.561 | 0 | 0 | 11.160 | 0 | 0 | 0 |
| NT2NE2011154 | 100 | 0 | 0 | 0 | 0 | 0 | 0 |
| NT2NE2011221 | 52.138 | 0 | 0 | 0 | 0 | 0 | 0 |
| NT2NE2011411 | 100 | 0 | 0 | 0 | 0 | 0 | 0 |
| NT2NE2011485 | 68.667 | 0 | 0 | 0 | 0 | 0 | 0 |
| NT2NE2011650 | 66.603 | 33.397 | 0 | 0 | 0 | 0 | 0 |
| NT2NE2011684 | 31.736 | 0 | 0 | 0 | 0 | 0 | 0 |
| NT2NE2011687 | 100 | 0 | 0 | 0 | 0 | 0 | 0 |
| NT2NE2011691 | 100 | 0 | 0 | 0 | 0 | 0 | 0 |
| NT2NE2011758 | 100 | 0 | 0 | 0 | 0 | 0 | 0 |
| NT2NE2011823 | 100 | 0 | 0 | 0 | 0 | 0 | 0 |
| NT2NE2011896 | 100 | 0 | 0 | 0 | 0 | 0 | 0 |
| NT2NE2011998 | 74.472 | 0 | 0 | 0 | 0 | 0 | 25.528 |
| NT2NE2012113 | 100 | 0 | 0 | 0 | 0 | 0 | 0 |
| NT2NE2012199 | 87.795 | 0 | 0 | 0 | 0 | 0 | 0 |
| NT2NE2012243 | 76.570 | 0 | 0 | 0 | 0 | 0 | 0 |
| NT2NE2012361 | 100 | 0 | 0 | 0 | 0 | 0 | 0 |
| NT2NE2012448 | 100 | 0 | 0 | 0 | 0 | 0 | 0 |
| NT2NE2012457 | 100 | 0 | 0 | 0 | 0 | 0 | 0 |
| NT2NE2012493 | 100 | 0 | 0 | 0 | 0 | 0 | 0 |
| NT2NE2012505 | 66.449 | 0 | 0 | 0 | 0 | 0 | 0 |
| NT2NE2012533 | 100 | 0 | 0 | 0 | 0 | 0 | 0 |
| NT2NE2012603 | 100 | 0 | 0 | 0 | 0 | 0 | 0 |
| NT2NE2012747 | 100 | 0 | 0 | 0 | 0 | 0 | 0 |
| NT2NE2012790 | 21.741 | 10.901 | 0 | 0 | 0 | 0 | 0 |
| NT2NE2012847 | 100 | 0 | 0 | 0 | 0 | 0 | 0 |
| NT2NE2012928 | 31.553 | 0 | 0 | 0 | 0 | 0 | 0 |
| NT2NE2013019 | 66.603 | 33.397 | 0 | 0 | 0 | 0 | 0 |
| NT2NE2013081 | 100 | 0 | 0 | 0 | 0 | 0 | 0 |
| NT2NE2013189 | 100 | 0 | 0 | 0 | 0 | 0 | 0 |
| NT2NE2013217 | 100 | 0 | 0 | 0 | 0 | 0 | 0 |
| NT2NE2013547 | 100 | 0 | 0 | 0 | 0 | 0 | 0 |
| NT2NE2014013 | 100 | 0 | 0 | 0 | 0 | 0 | 0 |
| NT2NE2014028 | 100 | 0 | 0 | 0 | 0 | 0 | 0 |
| NT2NE2014104 | 6.083 | 0 | 0 | 0 | 0 | 0 | 0 |
| NT2NE2014113 | 32.885 | 0 | 0 | 0 | 0 | 0 | 0 |
| NT2NE2014176 | 49.929 | 50.071 | 0 | 0 | 0 | 0 | 0 |
| NT2NE2014221 | 100 | 0 | 0 | 0 | 0 | 0 | 0 |
| NT2NE2014525 | 100 | 0 | 0 | 0 | 0 | 0 | 0 |
| NT2NE2014650 | 100 | 0 | 0 | 0 | 0 | 0 | 0 |
| NT2NE2014651 | 100 | 0 | 0 | 0 | 0 | 0 | 0 |
| NT2NE2014681 | 100 | 0 | 0 | 0 | 0 | 0 | 0 |
| NT2NE2014758 | 100 | 0 | 0 | 0 | 0 | 0 | 0 |
| NT2NE2014869 | 100 | 0 | 0 | 0 | 0 | 0 | 0 |
| NT2NE2014950 | 100 | 0 | 0 | 0 | 0 | 0 | 0 |
| NT2NE2015061 | 100 | 0 | 0 | 0 | 0 | 0 | 0 |
| NT2NE2015262 | 81.295 | 0 | 0 | 0 | 0 | 0 | 0 |
| NT2NE2015275 | 100 | 0 | 0 | 0 | 0 | 0 | 0 |
| NT2NE2015362 | 66.449 | 0 | 0 | 0 | 0 | 0 | 0 |
| NT2NE2015480 | 100 | 0 | 0 | 0 | 0 | 0 | 0 |
| NT2NE2015511 | 100 | 0 | 0 | 0 | 0 | 0 | 0 |
| NT2NE2015565 | 100 | 0 | 0 | 0 | 0 | 0 | 0 |
| NT2NE2015626 | 100 | 0 | 0 | 0 | 0 | 0 | 0 |
| NT2NE2015747 | 100 | 0 | 0 | 0 | 0 | 0 | 0 |
| NT2NE2015860 | 66.603 | 33.397 | 0 | 0 | 0 | 0 | 0 |
| NT2NE2015885 | 100 | 0 | 0 | 0 | 0 | 0 | 0 |
| NT2NE2015904 | 78.033 | 0 | 0 | 0 | 0 | 0 | 0 |
| NT2NE2015974 | 27.684 | 0 | 0 | 0 | 0 | 0 | 18.979 |
| NT2NE2016041 | 100 | 0 | 0 | 0 | 0 | 0 | 0 |
| NT2NE2016387 | 78.374 | 0 | 0 | 0 | 0 | 0 | 0 |
| NT2NE2016419 | 100 | 0 | 0 | 0 | 0 | 0 | 0 |
| NT2NE2016519 | 100 | 0 | 0 | 0 | 0 | 0 | 0 |
| NT2NE2016608 | 100 | 0 | 0 | 0 | 0 | 0 | 0 |
| NT2NE2016766 | 100 | 0 | 0 | 0 | 0 | 0 | 0 |
| NT2NE2016774 | 100 | 0 | 0 | 0 | 0 | 0 | 0 |
| NT2NE2016971 | 49.619 | 24.880 | 0 | 0 | 0 | 0 | 0 |
| NT2NE2017259 | 100 | 0 | 0 | 0 | 0 | 0 | 0 |
| NT2NE2017332 | 100 | 0 | 0 | 0 | 0 | 0 | 0 |
| NT2NE2017397 | 100 | 0 | 0 | 0 | 0 | 0 | 0 |
| NT2NE2017480 | 60.059 | 0 | 0 | 39.941 | 0 | 0 | 0 |
| NT2NE2017492 | 61.159 | 0 | 0 | 0 | 0 | 0 | 0 |
| NT2NE2017508 | 100 | 0 | 0 | 0 | 0 | 0 | 0 |
| NT2NE2017562 | 100 | 0 | 0 | 0 | 0 | 0 | 0 |
| NT2NE2017590 | 100 | 0 | 0 | 0 | 0 | 0 | 0 |
| NT2NE2017612 | 100 | 0 | 0 | 0 | 0 | 0 | 0 |
| NT2NE2017721 | 100 | 0 | 0 | 0 | 0 | 0 | 0 |
| NT2NE2017752 | 42.542 | 0 | 0 | 28.292 | 0 | 0 | 29.166 |
| NT2NE2017792 | 67.849 | 0 | 0 | 0 | 0 | 0 | 0 |
| NT2NE2017982 | 84.719 | 0 | 0 | 0 | 0 | 0 | 0 |
| NT2NE2018165 | 100 | 0 | 0 | 0 | 0 | 0 | 0 |
| NT2NE2018176 | 100 | 0 | 0 | 0 | 0 | 0 | 0 |
| NT2NE2018180 | 13.627 | 0 | 0 | 0 | 0 | 0 | 0 |
| NT2NE2018273 | 100 | 0 | 0 | 0 | 0 | 0 | 0 |
| NT2NE2018376 | 100 | 0 | 0 | 0 | 0 | 0 | 0 |
| NT2NE2018472 | 100 | 0 | 0 | 0 | 0 | 0 | 0 |
| NT2NE2018490 | 84.719 | 0 | 0 | 0 | 0 | 0 | 0 |
| NT2NE2018594 | 61.235 | 0 | 0 | 0 | 0 | 0 | 0 |
| NT2NE2018739 | 100 | 0 | 0 | 0 | 0 | 0 | 0 |
| NT2NE2018916 | 100 | 0 | 0 | 0 | 0 | 0 | 0 |
| NT2NE2019056 | 100 | 0 | 0 | 0 | 0 | 0 | 0 |
| NT2NE2019076 | 32.654 | 0 | 0 | 0 | 0 | 0 | 0 |
| NT2NE2019091 | 6.861 | 0 | 0 | 9.125 | 0 | 0 | 4.704 |
| NT2NE2019099 | 100 | 0 | 0 | 0 | 0 | 0 | 0 |
| NT2RI1000016 | 0 | 45.918 | 0 | 0 | 0 | 0 | 0 |
| NT2RI1000027 | 0 | 1.837 | 0 | 2.437 | 0 | 0 | 0 |
| NT2RI1000035 | 0 | 34.405 | 0 | 0 | 0 | 0 | 0 |
| NT2RI1000048 | 6.154 | 1.543 | 0 | 0 | 0 | 0 | 1.055 |
| NT2RI1000127 | 6.578 | 3.298 | 0 | 0 | 0 | 0 | 4.509 |
| NT2RI1000164 | 0 | 100 | 0 | 0 | 0 | 0 | 0 |
| NT2RI1000172 | 46.158 | 23.145 | 0 | 30.696 | 0 | 0 | 0 |
| NT2RI2000004 | 0 | 100 | 0 | 0 | 0 | 0 | 0 |
| NT2RI2000007 | 0 | 60.375 | 0 | 0 | 0 | 0 | 0 |
| NT2RI2000064 | 3.901 | 0.978 | 0 | 2.594 | 0 | 0 | 0.669 |
| NT2RI2000107 | 0 | 8.418 | 0 | 5.583 | 0 | 0 | 0 |
| NT2RI2000116 | 0 | 4.227 | 0 | 8.410 | 0 | 0 | 50.573 |
| NT2RI2000117 | 0 | 100 | 0 | 0 | 0 | 0 | 0 |
| NT2RI2000128 | 0 | 100 | 0 | 0 | 0 | 0 | 0 |
| NT2RI2000133 | 9.286 | 4.656 | 0 | 0 | 0 | 0 | 0 |
| NT2RI2000167 | 0 | 20.995 | 0 | 0 | 0 | 0 | 0 |
| NT2RI2000173 | 0 | 42.988 | 0 | 57.012 | 0 | 0 | 0 |
| NT2RI2000187 | 0 | 100 | 0 | 0 | 0 | 0 | 0 |
| NT2RI2000255 | 4.338 | 2.175 | 0 | 0 | 0 | 0 | 0 |
| NT2RI2000263 | 6.199 | 6.217 | 0 | 4.123 | 0 | 0 | 6.375 |
| NT2RI2000270 | 0 | 100 | 0 | 0 | 0 | 0 | 0 |
| NT2RI2000276 | 0 | 100 | 0 | 0 | 0 | 0 | 0 |
| NT2RI2000282 | 1.147 | 3.451 | 0 | 2.289 | 0 | 0 | 0 |
| NT2RI2000284 | 0 | 0.828 | 0 | 0 | 0 | 0 | 0 |
| NT2RI2000294 | 6.859 | 10.318 | 0 | 4.562 | 0 | 0 | 7.054 |
| NT2RI2000313 | 0 | 33.224 | 0 | 44.064 | 0 | 0 | 22.712 |
| NT2RI2000341 | 0 | 100 | 0 | 0 | 0 | 0 | 0 |
| NT2RI2000344 | 5.868 | 1.471 | 0 | 0 | 0 | 0 | 1.006 |
| NT2RI2000348 | 0 | 32.207 | 0 | 0 | 0 | 0 | 44.033 |
| NT2RI2000412 | 0 | 28.013 | 0 | 0 | 0 | 0 | 9.575 |
| NT2RI2000421 | 11.028 | 11.060 | 0 | 0 | 0 | 0 | 0 |
| NT2RI2000480 | 0 | 100 | 0 | 0 | 0 | 0 | 0 |
| NT2RI2000575 | 0 | 100 | 0 | 0 | 0 | 0 | 0 |
| NT2RI2000578 | 1.212 | 1.823 | 0 | 0.806 | 0 | 0 | 0 |
| NT2RI2000588 | 0 | 100 | 0 | 0 | 0 | 0 | 0 |
| NT2RI2000597 | 0 | 100 | 0 | 0 | 0 | 0 | 0 |
| NT2RI2000669 | 0 | 22.312 | 0 | 0 | 0 | 0 | 0 |
| NT2RI2000671 | 3.812 | 1.911 | 0 | 7.605 | 0 | 0 | 0 |
| NT2RI2000685 | 7.181 | 1.800 | 0 | 0 | 0 | 7.302 | 1.231 |
| NT2RI2000688 | 0 | 100 | 0 | 0 | 0 | 0 | 0 |
| NT2RI2000689 | 0 | 11.240 | 0 | 3.727 | 0 | 11.396 | 1.921 |
| NT2RI2000704 | 0 | 100 | 0 | 0 | 0 | 0 | 0 |
| NT2RI2000727 | 0.938 | 0.940 | 0 | 0 | 0 | 0 | 0.321 |
| NT2RI2000738 | 0 | 100 | 0 | 0 | 0 | 0 | 0 |
| NT2RI2000775 | 0 | 61.984 | 0 | 0 | 0 | 0 | 0 |
| NT2RI2000822 | 0 | 19.359 | 0 | 0 | 0 | 0 | 0 |
| NT2RI2000829 | 13.842 | 6.941 | 0 | 0 | 0 | 0 | 14.234 |
| NT2RI2000841 | 0 | 100 | 0 | 0 | 0 | 0 | 0 |
| NT2RI2000865 | 0 | 5.097 | 0 | 0 | 0 | 0 | 0 |
| NT2RI2000932 | 0 | 100 | 0 | 0 | 0 | 0 | 0 |
| NT2RI2000974 | 7.817 | 11.759 | 0 | 10.397 | 0 | 0 | 2.680 |
| NT2RI2000987 | 0 | 100 | 0 | 0 | 0 | 0 | 0 |
| NT2RI2001010 | 0 | 68.547 | 0 | 0 | 0 | 0 | 0 |
| NT2RI2001017 | 0 | 100 | 0 | 0 | 0 | 0 | 0 |
| NT2RI2001030 | 0 | 59.396 | 0 | 0 | 0 | 0 | 40.604 |
| NT2RI2001054 | 2.156 | 1.081 | 0 | 0 | 0 | 0 | 3.696 |
| NT2RI2001083 | 0 | 100 | 0 | 0 | 0 | 0 | 0 |
| NT2RI2001091 | 0 | 6.389 | 0 | 0 | 0 | 0 | 0 |
| NT2RI2001167 | 0 | 73.545 | 0 | 0 | 0 | 0 | 0 |
| NT2RI2001178 | 0 | 100 | 0 | 0 | 0 | 0 | 0 |
| NT2RI2001230 | 0 | 5.607 | 0 | 0 | 0 | 0 | 0 |
| NT2RI2001235 | 0 | 100 | 0 | 0 | 0 | 0 | 0 |
| NT2RI2001244 | 0 | 100 | 0 | 0 | 0 | 0 | 0 |
| NT2RI2001307 | 0 | 100 | 0 | 0 | 0 | 0 | 0 |
| NT2RI2001342 | 0 | 2.868 | 0 | 0 | 0 | 0 | 1.961 |
| NT2RI2001385 | 0 | 7.160 | 0 | 0 | 0 | 0 | 0 |
| NT2RI2001409 | 0 | 20.927 | 0 | 0 | 0 | 0 | 0 |
| NT2RI2001410 | 0 | 100 | 0 | 0 | 0 | 0 | 0 |
| NT2RI2001422 | 0 | 6.756 | 0 | 0 | 0 | 0 | 0 |
| NT2RI2001425 | 0 | 100 | 0 | 0 | 0 | 0 | 0 |
| NT2RI2001449 | 0 | 72.520 | 0 | 27.480 | 0 | 0 | 0 |
| NT2RI2001450 | 6.421 | 3.220 | 0 | 0 | 0 | 0 | 8.805 |
| NT2RI2001474 | 0 | 100 | 0 | 0 | 0 | 0 | 0 |
| NT2RI2001519 | 0 | 100 | 0 | 0 | 0 | 0 | 0 |
| NT2RI2001540 | 0.413 | 0.517 | 1.638 | 0.274 | 0 | 0 | 0.071 |
| NT2RI2001595 | 0 | 14.429 | 0 | 57.411 | 0 | 0 | 0 |
| NT2RI2001621 | 11.094 | 11.125 | 0 | 0 | 0 | 0 | 3.803 |
| NT2RI2001624 | 0 | 100 | 0 | 0 | 0 | 0 | 0 |
| NT2RI2001657 | 0 | 0.972 | 0 | 0 | 0 | 0 | 0 |
| NT2RI2001726 | 0 | 38.377 | 0 | 0 | 0 | 0 | 13.117 |
| NT2RI2001731 | 6.864 | 3.442 | 0 | 0 | 0 | 0 | 4.705 |
| NT2RI2001769 | 0 | 16.159 | 0 | 0 | 0 | 0 | 0 |
| NT2RI2001782 | 0 | 100 | 0 | 0 | 0 | 0 | 0 |
| NT2RI2001836 | 0 | 100 | 0 | 0 | 0 | 0 | 0 |
| NT2RI2001846 | 0 | 8.955 | 0 | 0 | 0 | 0 | 0 |
| NT2RI2001859 | 3.517 | 3.527 | 0 | 0 | 0 | 7.152 | 2.411 |
| NT2RI2001866 | 0 | 26.371 | 0 | 34.974 | 0 | 0 | 0 |
| NT2RI2001910 | 0 | 13.573 | 0 | 0 | 0 | 0 | 0 |
| NT2RI2001952 | 0 | 38.068 | 0 | 0 | 0 | 0 | 0 |
| NT2RI2002022 | 0 | 59.396 | 0 | 0 | 0 | 0 | 40.604 |
| NT2RI2002041 | 0 | 100 | 0 | 0 | 0 | 0 | 0 |
| NT2RI2002091 | 0.389 | 0.756 | 0 | 0.065 | 0 | 2.867 | 0 |
| NT2RI2002117 | 0 | 8.561 | 0 | 0 | 0 | 0 | 0 |
| NT2RI2002120 | 0 | 100 | 0 | 0 | 0 | 0 | 0 |
| NT2RI2002152 | 0 | 9.141 | 0 | 0 | 0 | 0 | 0 |
| NT2RI2002153 | 0 | 100 | 0 | 0 | 0 | 0 | 0 |
| NT2RI2002179 | 0 | 100 | 0 | 0 | 0 | 0 | 0 |
| NT2RI2002243 | 0 | 5.978 | 0 | 0 | 0 | 0 | 0 |
| NT2RI2002252 | 0 | 100 | 0 | 0 | 0 | 0 | 0 |
| NT2RI2002260 | 0 | 42.988 | 0 | 57.012 | 0 | 0 | 0 |
| NT2RI2002266 | 0 | 4.555 | 72.122 | 0 | 0 | 0 | 6.227 |
| NT2RI2002270 | 0 | 100 | 0 | 0 | 0 | 0 | 0 |
| NT2RI2002316 | 0 | 1.457 | 0 | 1.932 | 0 | 0 | 0.996 |
| NT2RI2002334 | 0 | 100 | 0 | 0 | 0 | 0 | 0 |
| NT2RI2002359 | 0 | 29.911 | 0 | 0 | 0 | 0 | 0 |
| NT2RI2002391 | 0 | 11.790 | 0 | 0 | 0 | 0 | 1.007 |
| NT2RI2002447 | 0 | 100 | 0 | 0 | 0 | 0 | 0 |
| NT2RI2002481 | 0 | 100 | 0 | 0 | 0 | 0 | 0 |
| NT2RI2002507 | 0 | 100 | 0 | 0 | 0 | 0 | 0 |
| NT2RI2002517 | 0 | 42.988 | 0 | 57.012 | 0 | 0 | 0 |
| NT2RI2002530 | 0 | 10.969 | 0 | 0 | 0 | 0 | 7.499 |
| NT2RI2002540 | 6.265 | 3.141 | 0 | 0 | 0 | 0 | 2.148 |
| NT2RI2002549 | 0 | 12.521 | 0 | 0 | 0 | 0 | 8.559 |
| NT2RI2002554 | 6.571 | 3.295 | 0 | 4.370 | 0 | 0 | 9.010 |
| NT2RI2002564 | 0 | 100 | 0 | 0 | 0 | 0 | 0 |
| NT2RI2002585 | 0 | 23.627 | 0 | 0 | 0 | 0 | 16.152 |
| NT2RI2002592 | 0 | 33.510 | 0 | 0 | 0 | 0 | 0 |
| NT2RI2002602 | 0 | 14.231 | 0 | 56.622 | 0 | 0 | 0 |
| NT2RI2002654 | 2.129 | 4.270 | 0 | 2.832 | 0 | 4.329 | 0.730 |
| NT2RI2002699 | 0 | 20.778 | 0 | 0 | 0 | 0 | 0 |
| NT2RI2002729 | 0 | 7.644 | 0 | 0 | 0 | 0 | 0 |
| NT2RI2002802 | 0 | 52.242 | 0 | 0 | 0 | 0 | 0 |
| NT2RI2002819 | 0 | 6.766 | 0 | 8.973 | 0 | 0 | 4.625 |
| NT2RI2002822 | 0 | 9.118 | 0 | 0 | 0 | 0 | 0 |
| NT2RI2002847 | 4.549 | 6.843 | 0 | 9.076 | 0 | 9.251 | 7.797 |
| NT2RI2002852 | 0 | 73.545 | 0 | 0 | 0 | 0 | 0 |
| NT2RI2002865 | 0 | 24.645 | 0 | 0 | 0 | 0 | 0 |
| NT2RI2002923 | 0 | 100 | 0 | 0 | 0 | 0 | 0 |
| NT2RI2002926 | 57.202 | 14.342 | 0 | 0 | 0 | 0 | 0 |
| NT2RI2002958 | 0 | 5.249 | 0 | 0 | 0 | 0 | 8.372 |
| NT2RI2002970 | 0 | 100 | 0 | 0 | 0 | 0 | 0 |
| NT2RI2003011 | 12.513 | 6.275 | 0 | 0 | 0 | 0 | 0 |
| NT2RI2003019 | 0 | 19.008 | 0 | 0 | 0 | 0 | 0 |
| NT2RI2003051 | 0 | 1.860 | 0 | 4.935 | 0 | 0 | 0 |
| NT2RI2003067 | 0 | 100 | 0 | 0 | 0 | 0 | 0 |
| NT2RI2003184 | 0 | 34.265 | 0 | 0 | 0 | 0 | 0 |
| NT2RI2003205 | 0 | 17.549 | 0 | 0 | 0 | 0 | 31.992 |
| NT2RI2003222 | 0 | 46.196 | 0 | 0 | 0 | 0 | 0 |
| NT2RI2003301 | 0 | 10.314 | 0 | 0 | 0 | 0 | 42.305 |
| NT2RI2003304 | 1.902 | 4.769 | 0 | 4.427 | 0 | 0 | 0.978 |
| NT2RI2003317 | 0 | 2.864 | 0 | 3.799 | 0 | 0 | 13.707 |
| NT2RI2003338 | 0 | 7.360 | 0 | 2.440 | 0 | 0 | 5.031 |
| NT2RI2003344 | 0 | 3.539 | 0 | 0 | 0 | 0 | 0 |
| NT2RI2003360 | 0 | 100 | 0 | 0 | 0 | 0 | 0 |
| NT2RI2003361 | 17.898 | 8.975 | 0 | 11.903 | 0 | 0 | 0 |
| NT2RI2003383 | 0 | 32.968 | 0 | 0 | 0 | 0 | 0 |
| NT2RI2003392 | 0 | 100 | 0 | 0 | 0 | 0 | 0 |
| NT2RI2003399 | 0 | 58.159 | 0 | 0 | 0 | 0 | 0 |
| NT2RI2003407 | 0 | 2.199 | 0 | 0 | 0 | 0 | 1.504 |
| NT2RI2003419 | 0 | 20.514 | 0 | 0 | 0 | 0 | 0 |
| NT2RI2003420 | 5.581 | 11.194 | 0 | 3.711 | 0 | 0 | 0 |
| NT2RI2003481 | 0 | 3.509 | 0 | 0 | 0 | 0 | 4.798 |
| NT2RI2003487 | 0 | 6.789 | 0 | 0 | 0 | 0 | 0 |
| NT2RI2003556 | 1.379 | 0.691 | 0 | 3.668 | 0 | 0 | 1.418 |
| NT2RI2003655 | 0 | 35.500 | 0 | 0 | 0 | 0 | 0 |
| NT2RI2003667 | 66.603 | 33.397 | 0 | 0 | 0 | 0 | 0 |
| NT2RI2003678 | 0 | 13.840 | 0 | 9.178 | 0 | 0 | 4.731 |
| NT2RI2003695 | 2.854 | 5.724 | 0 | 1.898 | 0 | 0 | 2.935 |
| NT2RI2003738 | 0.634 | 0.636 | 0 | 0.422 | 0 | 0 | 1.087 |
| NT2RI2003751 | 10.041 | 5.035 | 0 | 0 | 0 | 0 | 0 |
| NT2RI2003884 | 0 | 100 | 0 | 0 | 0 | 0 | 0 |
| NT2RI2003921 | 6.875 | 4.309 | 0 | 4.572 | 0 | 0 | 0 |
| NT2RI2003973 | 0 | 45.622 | 0 | 0 | 0 | 0 | 31.188 |
| NT2RI2003993 | 0 | 6.899 | 0 | 0 | 0 | 0 | 0 |
| NT2RI2004059 | 0 | 12.383 | 0 | 0 | 0 | 0 | 0 |
| NT2RI2004078 | 0 | 64.504 | 0 | 0 | 0 | 0 | 0 |
| NT2RI2004079 | 0 | 100 | 0 | 0 | 0 | 0 | 0 |
| NT2RI2004093 | 0 | 100 | 0 | 0 | 0 | 0 | 0 |
| NT2RI2004136 | 0 | 100 | 0 | 0 | 0 | 0 | 0 |
| NT2RI2004157 | 0 | 60.375 | 0 | 0 | 0 | 0 | 0 |
| NT2RI2004188 | 0 | 10.436 | 0 | 0 | 0 | 0 | 0 |
| NT2RI2004190 | 0 | 100 | 0 | 0 | 0 | 0 | 0 |
| NT2RI2004209 | 0 | 13.341 | 0 | 0 | 0 | 0 | 0 |
| NT2RI2004230 | 12.475 | 6.255 | 0 | 19.358 | 0 | 0 | 17.105 |
| NT2RI2004284 | 0 | 2.921 | 0 | 0 | 0 | 0 | 0 |
| NT2RI2004290 | 0 | 38.769 | 0 | 0 | 0 | 0 | 0 |
| NT2RI2004304 | 0 | 7.568 | 0 | 10.036 | 0 | 0 | 20.693 |
| NT2RI2004312 | 0 | 27.584 | 0 | 0 | 0 | 0 | 18.857 |
| NT2RI2004381 | 0 | 100 | 0 | 0 | 0 | 0 | 0 |
| NT2RI2004398 | 0 | 9.053 | 0 | 0 | 0 | 0 | 12.377 |
| NT2RI2004410 | 18.077 | 9.065 | 0 | 6.011 | 0 | 0 | 0 |
| NT2RI2004442 | 0 | 100 | 0 | 0 | 0 | 0 | 0 |
| NT2RI2004468 | 0 | 100 | 0 | 0 | 0 | 0 | 0 |
| NT2RI2004535 | 6.937 | 13.914 | 0 | 0 | 0 | 0 | 0 |
| NT2RI2004606 | 0 | 64.504 | 0 | 0 | 0 | 0 | 0 |
| NT2RI2004608 | 0 | 6.723 | 0 | 0 | 0 | 0 | 0 |
| NT2RI2004618 | 0 | 100 | 0 | 0 | 0 | 0 | 0 |
| NT2RI2004783 | 0 | 78.423 | 0 | 0 | 0 | 0 | 0 |
| NT2RI2004818 | 0 | 100 | 0 | 0 | 0 | 0 | 0 |
| NT2RI2004840 | 0 | 100 | 0 | 0 | 0 | 0 | 0 |
| NT2RI2004884 | 5.600 | 1.404 | 0 | 0 | 0 | 0 | 0.960 |
| NT2RI2004916 | 0 | 60.128 | 0 | 39.872 | 0 | 0 | 0 |
| NT2RI2004962 | 0 | 100 | 0 | 0 | 0 | 0 | 0 |
| NT2RI2004984 | 0 | 100 | 0 | 0 | 0 | 0 | 0 |
| NT2RI2004985 | 0 | 100 | 0 | 0 | 0 | 0 | 0 |
| NT2RI2005048 | 0 | 100 | 0 | 0 | 0 | 0 | 0 |
| NT2RI2005061 | 0 | 15.855 | 0 | 21.027 | 0 | 0 | 5.419 |
| NT2RI2005069 | 0 | 100 | 0 | 0 | 0 | 0 | 0 |
| NT2RI2005087 | 0 | 7.791 | 0 | 0 | 0 | 0 | 0 |
| NT2RI2005096 | 0 | 73.545 | 0 | 0 | 0 | 0 | 0 |
| NT2RI2005115 | 0 | 11.105 | 0 | 0 | 0 | 0 | 0 |
| NT2RI2005116 | 0 | 4.646 | 0 | 3.081 | 0 | 0 | 4.764 |
| NT2RI2005130 | 49.929 | 50.071 | 0 | 0 | 0 | 0 | 0 |
| NT2RI2005150 | 7.492 | 2.504 | 0 | 1.661 | 0 | 0 | 1.712 |
| NT2RI2005166 | 0 | 47.393 | 0 | 0 | 0 | 0 | 0 |
| NT2RI2005239 | 14.292 | 21.500 | 0 | 19.009 | 0 | 0 | 4.899 |
| NT2RI2005246 | 0 | 16.871 | 0 | 0 | 0 | 0 | 11.533 |
| NT2RI2005315 | 0 | 49.384 | 0 | 0 | 0 | 0 | 0 |
| NT2RI2005335 | 1.403 | 2.814 | 0 | 4.665 | 0 | 0 | 1.924 |
| NT2RI2005353 | 0 | 6.280 | 0 | 0 | 0 | 0 | 0 |
| NT2RI2005358 | 0 | 37.296 | 0 | 0 | 0 | 0 | 0 |
| NT2RI2005368 | 0 | 7.344 | 0 | 4.870 | 0 | 0 | 17.571 |
| NT2RI2005382 | 0 | 35.947 | 0 | 0 | 0 | 0 | 0 |
| NT2RI2005405 | 4.069 | 1.020 | 0 | 2.030 | 0 | 2.069 | 0.349 |
| NT2RI2005473 | 0 | 5.935 | 0 | 0 | 0 | 0 | 0 |
| NT2RI2005520 | 0 | 100 | 0 | 0 | 0 | 0 | 0 |
| NT2RI2005564 | 16.142 | 16.188 | 0 | 0 | 0 | 0 | 16.600 |
| NT2RI2005579 | 0 | 17.054 | 0 | 0 | 0 | 0 | 11.659 |
| NT2RI2005621 | 0 | 100 | 0 | 0 | 0 | 0 | 0 |
| NT2RI2005628 | 0 | 100 | 0 | 0 | 0 | 0 | 0 |
| NT2RI2005647 | 0 | 1.202 | 0 | 0 | 0 | 82.851 | 0 |
| NT2RI2005670 | 0 | 100 | 0 | 0 | 0 | 0 | 0 |
| NT2RI2005710 | 0 | 18.971 | 0 | 37.741 | 0 | 0 | 0 |
| NT2RI2005713 | 0 | 2.687 | 0 | 0 | 0 | 0 | 3.674 |
| NT2RI2005723 | 7.275 | 0.912 | 0 | 1.209 | 0 | 0 | 0 |
| NT2RI2005772 | 0 | 100 | 0 | 0 | 0 | 0 | 0 |
| NT2RI2005811 | 0 | 8.352 | 0 | 0 | 0 | 11.291 | 1.903 |
| NT2RI2005814 | 0 | 2.367 | 0 | 6.278 | 0 | 0 | 0 |
| NT2RI2005816 | 0 | 11.615 | 0 | 15.405 | 0 | 0 | 47.643 |
| NT2RI2005818 | 9.230 | 13.885 | 0 | 0 | 0 | 0 | 12.656 |
| NT2RI2005851 | 0 | 13.544 | 0 | 17.963 | 0 | 0 | 13.888 |
| NT2RI2005966 | 0 | 33.881 | 0 | 0 | 0 | 0 | 0 |
| NT2RI2006070 | 0 | 3.735 | 0 | 0 | 0 | 0 | 0 |
| NT2RI2006071 | 0 | 5.130 | 0 | 6.804 | 0 | 0 | 7.014 |
| NT2RI2006072 | 2.773 | 2.781 | 0 | 3.688 | 0 | 0 | 1.901 |
| NT2RI2006081 | 0 | 100 | 0 | 0 | 0 | 0 | 0 |
| NT2RI2006126 | 0 | 100 | 0 | 0 | 0 | 0 | 0 |
| NT2RI2006127 | 0 | 21.950 | 0 | 0 | 0 | 0 | 0 |
| NT2RI2006183 | 0 | 17.381 | 0 | 23.051 | 0 | 0 | 11.882 |
| NT2RI2006257 | 0 | 100 | 0 | 0 | 0 | 0 | 0 |
| NT2RI2006271 | 10.122 | 5.076 | 0 | 0 | 0 | 0 | 0 |
| NT2RI2006294 | 0 | 100 | 0 | 0 | 0 | 0 | 0 |
| NT2RI2006345 | 0 | 100 | 0 | 0 | 0 | 0 | 0 |
| NT2RI2006403 | 0 | 100 | 0 | 0 | 0 | 0 | 0 |
| NT2RI2006412 | 0 | 1.490 | 0 | 0 | 0 | 0 | 0 |
| NT2RI2006445 | 0 | 100 | 0 | 0 | 0 | 0 | 0 |
| NT2RI2006487 | 0 | 100 | 0 | 0 | 0 | 0 | 0 |
| NT2RI2006506 | 0 | 24.481 | 0 | 0 | 0 | 0 | 16.735 |
| NT2RI2006553 | 0 | 13.442 | 0 | 0 | 0 | 0 | 0 |
| NT2RI2006565 | 0 | 10.859 | 0 | 43.204 | 0 | 0 | 0 |
| NT2RI2006640 | 0 | 22.843 | 0 | 15.148 | 0 | 0 | 0 |
| NT2RI2006647 | 0 | 8.333 | 0 | 0 | 0 | 0 | 5.696 |
| NT2RI2006659 | 0 | 42.988 | 0 | 57.012 | 0 | 0 | 0 |
| NT2RI2006662 | 0 | 100 | 0 | 0 | 0 | 0 | 0 |
| NT2RI2006667 | 0 | 100 | 0 | 0 | 0 | 0 | 0 |
| NT2RI2006679 | 0 | 100 | 0 | 0 | 0 | 0 | 0 |
| NT2RI2006682 | 0 | 35.773 | 0 | 23.722 | 0 | 0 | 0 |
| NT2RI2006686 | 0 | 21.246 | 0 | 0 | 0 | 0 | 0 |
| NT2RI2006703 | 0 | 29.452 | 0 | 39.061 | 0 | 0 | 0 |
| NT2RI2006716 | 0 | 100 | 0 | 0 | 0 | 0 | 0 |
| NT2RI2006735 | 0 | 28.601 | 0 | 0 | 0 | 0 | 0 |
| NT2RI2006762 | 0 | 100 | 0 | 0 | 0 | 0 | 0 |
| NT2RI2006788 | 0 | 100 | 0 | 0 | 0 | 0 | 0 |
| NT2RI2006825 | 0 | 4.982 | 0 | 0 | 0 | 0 | 3.406 |
| NT2RI2006838 | 0 | 100 | 0 | 0 | 0 | 0 | 0 |
| NT2RI2006853 | 8.129 | 4.076 | 0 | 0 | 0 | 0 | 0 |
| NT2RI2006855 | 0 | 15.665 | 0 | 0 | 0 | 0 | 0 |
| NT2RI2006856 | 5.093 | 0.851 | 0 | 1.129 | 0 | 0 | 0 |
| NT2RI2006943 | 17.631 | 8.841 | 0 | 2.931 | 0 | 0 | 3.022 |
| NT2RI2006973 | 0 | 100 | 0 | 0 | 0 | 0 | 0 |
| NT2RI2007046 | 0 | 100 | 0 | 0 | 0 | 0 | 0 |
| NT2RI2007048 | 0 | 100 | 0 | 0 | 0 | 0 | 0 |
| NT2RI2007054 | 0 | 2.359 | 0 | 0 | 0 | 0 | 1.613 |
| NT2RI2007084 | 0 | 1.977 | 0 | 0 | 0 | 0 | 0 |
| NT2RI2007096 | 0 | 9.420 | 0 | 12.494 | 0 | 0 | 19.320 |
| NT2RI2007116 | 0 | 100 | 0 | 0 | 0 | 0 | 0 |
| NT2RI2007133 | 1.422 | 1.426 | 0 | 1.891 | 0 | 0 | 0 |
| NT2RI2007148 | 0 | 1.716 | 0 | 0 | 0 | 0 | 0 |
| NT2RI2007214 | 0 | 42.988 | 0 | 57.012 | 0 | 0 | 0 |
| NT2RI2007277 | 3.553 | 5.345 | 0 | 7.088 | 0 | 0 | 26.793 |
| NT2RI2007303 | 0 | 100 | 0 | 0 | 0 | 0 | 0 |
| NT2RI2007334 | 45.725 | 22.928 | 0 | 0 | 0 | 0 | 31.347 |
| NT2RI2007377 | 8.489 | 2.128 | 0 | 2.823 | 0 | 0 | 8.730 |
| NT2RI2007384 | 0 | 2.361 | 12.461 | 1.044 | 0 | 6.383 | 0 |
| NT2RI2007386 | 0 | 100 | 0 | 0 | 0 | 0 | 0 |
| NT2RI2007406 | 66.603 | 33.397 | 0 | 0 | 0 | 0 | 0 |
| NT2RI2007439 | 66.603 | 33.397 | 0 | 0 | 0 | 0 | 0 |
| NT2RI2007445 | 6.498 | 6.517 | 0 | 8.643 | 0 | 0 | 0 |
| NT2RI2007468 | 15.143 | 15.186 | 0 | 10.070 | 0 | 0 | 5.191 |
| NT2RI2007469 | 0 | 58.159 | 0 | 0 | 0 | 0 | 0 |
| NT2RI2007498 | 1.478 | 17.786 | 0 | 1.966 | 0 | 0 | 0 |
| NT2RI2007507 | 0 | 100 | 0 | 0 | 0 | 0 | 0 |
| NT2RI2007572 | 2.708 | 1.358 | 0 | 3.602 | 0 | 0 | 7.426 |
| NT2RI2007589 | 0 | 3.881 | 0 | 5.148 | 0 | 0 | 0 |
| NT2RI2007593 | 0 | 29.937 | 0 | 0 | 0 | 0 | 0 |
| NT2RI2007629 | 0 | 14.653 | 0 | 0 | 0 | 0 | 5.008 |
| NT2RI2007723 | 12.821 | 12.858 | 0 | 0 | 0 | 0 | 0 |
| NT2RI2007729 | 0 | 20.986 | 0 | 0 | 0 | 0 | 0 |
| NT2RI2007751 | 0 | 100 | 0 | 0 | 0 | 0 | 0 |
| NT2RI2007754 | 0 | 35.266 | 0 | 0 | 0 | 0 | 0 |
| NT2RI2007827 | 0 | 13.642 | 0 | 0 | 0 | 0 | 0 |
| NT2RI2007853 | 0 | 8.137 | 0 | 0 | 0 | 0 | 0 |
| NT2RI2007877 | 0 | 100 | 0 | 0 | 0 | 0 | 0 |
| NT2RI2007879 | 0 | 5.768 | 0 | 0 | 0 | 0 | 0 |
| NT2RI2007884 | 0 | 4.613 | 0 | 12.237 | 0 | 0 | 6.308 |
| NT2RI2007891 | 0 | 100 | 0 | 0 | 0 | 0 | 0 |
| NT2RI2007956 | 0 | 100 | 0 | 0 | 0 | 0 | 0 |
| NT2RI2007965 | 0 | 42.988 | 0 | 57.012 | 0 | 0 | 0 |
| NT2RI2007987 | 0 | 6.544 | 0 | 0 | 0 | 0 | 13.421 |
| NT2RI2008007 | 0 | 35.172 | 0 | 0 | 0 | 0 | 0 |
| NT2RI2008045 | 0 | 1.411 | 0 | 1.871 | 0 | 0 | 3.858 |
| NT2RI2008050 | 0 | 100 | 0 | 0 | 0 | 0 | 0 |
| NT2RI2008141 | 0 | 7.772 | 0 | 20.614 | 0 | 0 | 5.313 |
| NT2RI2008171 | 0 | 8.069 | 0 | 0 | 0 | 0 | 0 |
| NT2RI2008188 | 0 | 52.242 | 0 | 0 | 0 | 0 | 0 |
| NT2RI2008204 | 44.987 | 11.279 | 0 | 0 | 0 | 0 | 0 |
| NT2RI2008221 | 0 | 2.015 | 0 | 0 | 0 | 0 | 0 |
| NT2RI2008233 | 0 | 34.405 | 0 | 0 | 0 | 0 | 0 |
| NT2RI2008336 | 1.452 | 3.640 | 0 | 2.897 | 0 | 0 | 1.991 |
| NT2RI2008396 | 4.792 | 5.607 | 0 | 4.249 | 0 | 0 | 2.738 |
| NT2RI2008455 | 0 | 100 | 0 | 0 | 0 | 0 | 0 |
| NT2RI2008481 | 0 | 4.032 | 0 | 3.565 | 0 | 5.451 | 0.919 |
| NT2RI2008502 | 0 | 100 | 0 | 0 | 0 | 0 | 0 |
| NT2RI2008526 | 0 | 2.787 | 0 | 0 | 0 | 5.650 | 0 |
| NT2RI2008566 | 0 | 17.092 | 0 | 0 | 0 | 0 | 11.684 |
| NT2RI2008598 | 0 | 15.244 | 0 | 0 | 0 | 0 | 0 |
| NT2RI2008622 | 0 | 5.735 | 0 | 7.606 | 0 | 23.258 | 3.920 |
| NT2RI2008656 | 0 | 4.935 | 78.152 | 0 | 0 | 0 | 0 |
| NT2RI2008714 | 0 | 26.665 | 0 | 0 | 0 | 0 | 0 |
| NT2RI2008724 | 0 | 3.337 | 0 | 0 | 0 | 0 | 0 |
| NT2RI2008749 | 4.098 | 1.027 | 0 | 1.363 | 0 | 0 | 0 |
| NT2RI2008791 | 4.228 | 6.361 | 0 | 0 | 0 | 8.599 | 0 |
| NT2RI2008801 | 0 | 100 | 0 | 0 | 0 | 0 | 0 |
| NT2RI2008808 | 16.429 | 8.238 | 0 | 10.926 | 0 | 0 | 0 |
| NT2RI2008812 | 0 | 13.156 | 0 | 0 | 0 | 0 | 0 |
| NT2RI2008841 | 0 | 100 | 0 | 0 | 0 | 0 | 0 |
| NT2RI2008942 | 0 | 13.339 | 0 | 27.801 | 0 | 0 | 3.908 |
| NT2RI2008952 | 0 | 100 | 0 | 0 | 0 | 0 | 0 |
| NT2RI2009005 | 0 | 100 | 0 | 0 | 0 | 0 | 0 |
| NT2RI2009037 | 3.055 | 4.596 | 0 | 0 | 0 | 0 | 4.189 |
| NT2RI2009065 | 1.693 | 4.244 | 0 | 1.126 | 0 | 0 | 2.321 |
| NT2RI2009066 | 19.866 | 4.981 | 0 | 6.606 | 0 | 0 | 0 |
| NT2RI2009083 | 0 | 18.633 | 0 | 0 | 0 | 0 | 0 |
| NT2RI2009101 | 0 | 100 | 0 | 0 | 0 | 0 | 0 |
| NT2RI2009103 | 0 | 58.159 | 0 | 0 | 0 | 0 | 0 |
| NT2RI2009144 | 0 | 100 | 0 | 0 | 0 | 0 | 0 |
| NT2RI2009151 | 0 | 100 | 0 | 0 | 0 | 0 | 0 |
| NT2RI2009173 | 4.042 | 2.027 | 0 | 2.688 | 0 | 0 | 1.386 |
| NT2RI2009194 | 0 | 5.358 | 0 | 0 | 0 | 0 | 9.767 |
| NT2RI2009215 | 0 | 100 | 0 | 0 | 0 | 0 | 0 |
| NT2RI2009233 | 0 | 4.099 | 0 | 5.436 | 0 | 0 | 5.604 |
| NT2RI2009239 | 0 | 5.080 | 0 | 3.369 | 0 | 0 | 0 |
| NT2RI2009259 | 0 | 21.268 | 0 | 0 | 0 | 0 | 0 |
| NT2RI2009269 | 21.488 | 7.183 | 0 | 2.382 | 0 | 0 | 0 |
| NT2RI2009281 | 5.101 | 2.558 | 0 | 10.176 | 0 | 0 | 1.748 |
| NT2RI2009289 | 0 | 100 | 0 | 0 | 0 | 0 | 0 |
| NT2RI2009301 | 0 | 100 | 0 | 0 | 0 | 0 | 0 |
| NT2RI2009402 | 0 | 5.700 | 0 | 0 | 0 | 0 | 19.482 |
| NT2RI2009406 | 10.196 | 20.451 | 0 | 13.561 | 0 | 0 | 0 |
| NT2RI2009517 | 0 | 100 | 0 | 0 | 0 | 0 | 0 |
| NT2RI2009583 | 0.811 | 0.813 | 0 | 0 | 0 | 4.947 | 0 |
| NT2RI2009585 | 0 | 100 | 0 | 0 | 0 | 0 | 0 |
| NT2RI2009595 | 0 | 100 | 0 | 0 | 0 | 0 | 0 |
| NT2RI2009855 | 0 | 100 | 0 | 0 | 0 | 0 | 0 |
| NT2RI2010283 | 0 | 13.449 | 0 | 35.673 | 0 | 0 | 0 |
| NT2RI2010508 | 0 | 100 | 0 | 0 | 0 | 0 | 0 |
| NT2RI2010795 | 0 | 29.973 | 0 | 0 | 0 | 0 | 0 |
| NT2RI2011422 | 0 | 100 | 0 | 0 | 0 | 0 | 0 |
| NT2RI2011450 | 0 | 46.154 | 0 | 0 | 0 | 0 | 0 |
| NT2RI2011683 | 0 | 100 | 0 | 0 | 0 | 0 | 0 |
| NT2RI2011803 | 0 | 100 | 0 | 0 | 0 | 0 | 0 |
| NT2RI2012350 | 0 | 2.182 | 0 | 0 | 0 | 0 | 0 |
| NT2RI2012659 | 0 | 24.645 | 0 | 0 | 0 | 0 | 0 |
| NT2RI2012726 | 16.813 | 8.430 | 0 | 11.181 | 0 | 0 | 5.763 |
| NT2RI2012990 | 0 | 24.360 | 0 | 0 | 0 | 0 | 0 |
| NT2RI2013345 | 0 | 17.187 | 0 | 0 | 0 | 0 | 0 |
| NT2RI2013357 | 0 | 73.545 | 0 | 0 | 0 | 0 | 0 |
| NT2RI2013373 | 0 | 100 | 0 | 0 | 0 | 0 | 0 |
| NT2RI2014247 | 0 | 27.718 | 0 | 0 | 0 | 0 | 0 |
| NT2RI2014551 | 0 | 100 | 0 | 0 | 0 | 0 | 0 |
| NT2RI2014683 | 0 | 100 | 0 | 0 | 0 | 0 | 0 |
| NT2RI2014733 | 0 | 45.319 | 0 | 0 | 0 | 0 | 0 |
| NT2RI2015239 | 0 | 100 | 0 | 0 | 0 | 0 | 0 |
| NT2RI2015342 | 0 | 8.201 | 0 | 10.877 | 0 | 0 | 0 |
| NT2RI2015533 | 0 | 2.824 | 0 | 0 | 0 | 0 | 3.861 |
| NT2RI2016128 | 0 | 100 | 0 | 0 | 0 | 0 | 0 |
| NT2RI2016134 | 0 | 100 | 0 | 0 | 0 | 0 | 0 |
| NT2RI2017529 | 0 | 3.526 | 0 | 0 | 0 | 7.151 | 0 |
| NT2RI2018311 | 0 | 78.082 | 0 | 0 | 0 | 0 | 0 |
| NT2RI2018363 | 0 | 100 | 0 | 0 | 0 | 0 | 0 |
| NT2RI2018448 | 0 | 11.442 | 0 | 15.176 | 0 | 0 | 0 |
| NT2RI2018883 | 0 | 49.384 | 0 | 0 | 0 | 0 | 0 |
| NT2RI2018950 | 0 | 100 | 0 | 0 | 0 | 0 | 0 |
| NT2RI2019751 | 0 | 71.913 | 0 | 0 | 0 | 0 | 0 |
| NT2RI2019826 | 0 | 100 | 0 | 0 | 0 | 0 | 0 |
| NT2RI2020390 | 0 | 100 | 0 | 0 | 0 | 0 | 0 |
| NT2RI2020703 | 0 | 100 | 0 | 0 | 0 | 0 | 0 |
| NT2RI2021463 | 0 | 100 | 0 | 0 | 0 | 0 | 0 |
| NT2RI2021625 | 0 | 34.630 | 0 | 45.928 | 0 | 0 | 0 |
| NT2RI2022468 | 0 | 12.182 | 0 | 0 | 0 | 0 | 0 |
| NT2RI2022584 | 0 | 100 | 0 | 0 | 0 | 0 | 0 |
| NT2RI2023303 | 0 | 100 | 0 | 0 | 0 | 0 | 0 |
| NT2RI2023671 | 0 | 100 | 0 | 0 | 0 | 0 | 0 |
| NT2RI2024008 | 0 | 61.866 | 0 | 0 | 0 | 0 | 0 |
| NT2RI2024313 | 0 | 26.626 | 0 | 0 | 0 | 0 | 0 |
| NT2RI2024460 | 0 | 100 | 0 | 0 | 0 | 0 | 0 |
| NT2RI2024496 | 0 | 100 | 0 | 0 | 0 | 0 | 0 |
| NT2RI2024935 | 0 | 7.895 | 0 | 0 | 0 | 0 | 0 |
| NT2RI2025075 | 0 | 30.809 | 0 | 0 | 0 | 0 | 21.061 |
| NT2RI2025255 | 0 | 62.102 | 0 | 0 | 0 | 0 | 0 |
| NT2RI2025564 | 0 | 100 | 0 | 0 | 0 | 0 | 0 |
| NT2RI2025565 | 0 | 100 | 0 | 0 | 0 | 0 | 0 |
| NT2RI2025909 | 0 | 100 | 0 | 0 | 0 | 0 | 0 |
| NT2RI2025957 | 0 | 100 | 0 | 0 | 0 | 0 | 0 |
| NT2RI2025976 | 0 | 100 | 0 | 0 | 0 | 0 | 0 |
| NT2RI2026758 | 0 | 100 | 0 | 0 | 0 | 0 | 0 |
| NT2RI2027081 | 0 | 100 | 0 | 0 | 0 | 0 | 0 |
| NT2RI2027157 | 0 | 100 | 0 | 0 | 0 | 0 | 0 |
| NT2RI2027224 | 0 | 61.866 | 0 | 0 | 0 | 0 | 0 |
| NT2RI2027323 | 0 | 60.375 | 0 | 0 | 0 | 0 | 0 |
| NT2RI2027396 | 0 | 100 | 0 | 0 | 0 | 0 | 0 |
| NT2RI2027484 | 0 | 100 | 0 | 0 | 0 | 0 | 0 |
| NT2RI2027975 | 0 | 71.913 | 0 | 0 | 0 | 0 | 0 |
| NT2RI2028213 | 0 | 100 | 0 | 0 | 0 | 0 | 0 |
| NT2RI2028222 | 0 | 100 | 0 | 0 | 0 | 0 | 0 |
| NT2RI2028537 | 0 | 100 | 0 | 0 | 0 | 0 | 0 |
| NT2RI2028642 | 0 | 100 | 0 | 0 | 0 | 0 | 0 |
| NT2RI3000174 | 0 | 61.866 | 0 | 0 | 0 | 0 | 0 |
| NT2RI3000213 | 0 | 100 | 0 | 0 | 0 | 0 | 0 |
| NT2RI3000423 | 0 | 8.738 | 0 | 34.67 | 0 | 0 | 0 |
| NT2RI3000622 | 0 | 15.558 | 0 | 0 | 0 | 63.099 | 0 |
| NT2RI3001132 | 0 | 100 | 0 | 0 | 0 | 0 | 0 |
| NT2RI3001263 | 0 | 100 | 0 | 0 | 0 | 0 | 0 |
| NT2RI3001279 | 0 | 100 | 0 | 0 | 0 | 0 | 0 |
| NT2RI3001348 | 0 | 18.992 | 0 | 0 | 0 | 0 | 0 |
| NT2RI3001445 | 0 | 11.509 | 0 | 0 | 0 | 0 | 7.868 |
| NT2RI3001458 | 0 | 100 | 0 | 0 | 0 | 0 | 0 |
| NT2RI3001515 | 0 | 19.682 | 0 | 0 | 0 | 0 | 40.364 |
| NT2RI3001573 | 0 | 100 | 0 | 0 | 0 | 0 | 0 |
| NT2RI3001967 | 0 | 100 | 0 | 0 | 0 | 0 | 0 |
| NT2RI3002303 | 0 | 100 | 0 | 0 | 0 | 0 | 0 |
| NT2RI3002557 | 0 | 100 | 0 | 0 | 0 | 0 | 0 |
| NT2RI3002842 | 0 | 34.054 | 0 | 45.164 | 0 | 0 | 0 |
| NT2RI3003027 | 0 | 100 | 0 | 0 | 0 | 0 | 0 |
| NT2RI3003031 | 0 | 100 | 0 | 0 | 0 | 0 | 0 |
| NT2RI3003095 | 0 | 100 | 0 | 0 | 0 | 0 | 0 |
| NT2RI3003104 | 0 | 28.425 | 0 | 0 | 0 | 0 | 0 |
| NT2RI3003162 | 0 | 100 | 0 | 0 | 0 | 0 | 0 |
| NT2RI3003362 | 0 | 100 | 0 | 0 | 0 | 0 | 0 |
| NT2RI3003382 | 0 | 71.913 | 0 | 0 | 0 | 0 | 0 |
| NT2RI3003409 | 0 | 100 | 0 | 0 | 0 | 0 | 0 |
| NT2RI3003738 | 0 | 32.959 | 0 | 0 | 0 | 0 | 0 |
| NT2RI3003925 | 0 | 64.044 | 0 | 0 | 0 | 0 | 0 |
| NT2RI3004133 | 0 | 100 | 0 | 0 | 0 | 0 | 0 |
| NT2RI3004381 | 0 | 100 | 0 | 0 | 0 | 0 | 0 |
| NT2RI3004510 | 0 | 51.230 | 0 | 0 | 0 | 0 | 0 |
| NT2RI3005202 | 0 | 100 | 0 | 0 | 0 | 0 | 0 |
| NT2RI3005403 | 0 | 62.102 | 0 | 0 | 0 | 0 | 0 |
| NT2RI3005416 | 0 | 100 | 0 | 0 | 0 | 0 | 0 |
| NT2RI3005724 | 0 | 10.770 | 0 | 14.284 | 0 | 0 | 0 |
| NT2RI3005861 | 0 | 100 | 0 | 0 | 0 | 0 | 0 |
| NT2RI3005923 | 0 | 35.423 | 0 | 0 | 0 | 0 | 0 |
| NT2RI3005928 | 0 | 100 | 0 | 0 | 0 | 0 | 0 |
| NT2RI3006132 | 0 | 100 | 0 | 0 | 0 | 0 | 0 |
| NT2RI3006171 | 0 | 100 | 0 | 0 | 0 | 0 | 0 |
| NT2RI3006284 | 0 | 3.760 | 0 | 0 | 0 | 0 | 0 |
| NT2RI3006340 | 0 | 14.839 | 0 | 0 | 0 | 0 | 0 |
| NT2RI3006376 | 0 | 100 | 0 | 0 | 0 | 0 | 0 |
| NT2RI3006666 | 0 | 37.941 | 0 | 0 | 0 | 0 | 0 |
| NT2RI3006673 | 0 | 18.135 | 0 | 0 | 0 | 0 | 0 |
| NT2RI3006796 | 0 | 100 | 0 | 0 | 0 | 0 | 0 |
| NT2RI3007065 | 0 | 100 | 0 | 0 | 0 | 0 | 0 |
| NT2RI3007095 | 0 | 100 | 0 | 0 | 0 | 0 | 0 |
| NT2RI3007158 | 0 | 100 | 0 | 0 | 0 | 0 | 0 |
| NT2RI3007167 | 0 | 100 | 0 | 0 | 0 | 0 | 0 |
| NT2RI3007213 | 0 | 71.913 | 0 | 0 | 0 | 0 | 0 |
| NT2RI3007291 | 0 | 22.746 | 0 | 0 | 0 | 0 | 0 |
| NT2RI3007443 | 0 | 100 | 0 | 0 | 0 | 0 | 0 |
| NT2RI3007543 | 6.150 | 1.542 | 0 | 0 | 0 | 6.253 | 3.162 |
| NT2RI3007684 | 0 | 100 | 0 | 0 | 0 | 0 | 0 |
| NT2RI3007757 | 0 | 45.964 | 0 | 30.480 | 0 | 0 | 0 |
| NT2RI3007878 | 0 | 100 | 0 | 0 | 0 | 0 | 0 |
| NT2RI3007978 | 0 | 100 | 0 | 0 | 0 | 0 | 0 |
| NT2RI3008055 | 0 | 40.142 | 0 | 0 | 0 | 0 | 0 |
| NT2RI3008162 | 0 | 100 | 0 | 0 | 0 | 0 | 0 |
| NT2RI3008179 | 0 | 100 | 0 | 0 | 0 | 0 | 0 |
| NT2RI3008228 | 0 | 100 | 0 | 0 | 0 | 0 | 0 |
| NT2RI3008442 | 0 | 100 | 0 | 0 | 0 | 0 | 0 |
| NT2RI3008652 | 0 | 100 | 0 | 0 | 0 | 0 | 0 |
| NT2RI3008697 | 0 | 100 | 0 | 0 | 0 | 0 | 0 |
| NT2RI3008974 | 0 | 100 | 0 | 0 | 0 | 0 | 0 |
| NT2RI3009158 | 0 | 45.727 | 0 | 0 | 0 | 0 | 0 |
| NT2RI3009480 | 0 | 68.547 | 0 | 0 | 0 | 0 | 0 |
| NT2RI3009524 | 0 | 64.504 | 0 | 0 | 0 | 0 | 0 |
| NT2RP6000005 | 0 | 0 | 0 | 5.301 | 0 | 0 | 5.465 |
| NT2RP6000008 | 0 | 0 | 0 | 77.250 | 0 | 0 | 0 |
| NT2RP6000017 | 5.422 | 0 | 0 | 3.606 | 0 | 0 | 1.859 |
| NT2RP6000032 | 0 | 14.251 | 0 | 18.901 | 0 | 0 | 0 |
| NT2RP6000033 | 0 | 0 | 0 | 10.025 | 0 | 0 | 0 |
| NT2RP6000039 | 0 | 3.259 | 0 | 8.644 | 0 | 0 | 6.683 |
| NT2RP6000043 | 13.765 | 0 | 0 | 9.154 | 0 | 0 | 4.718 |
| NT2RP6000059 | 0 | 0 | 0 | 52.276 | 0 | 0 | 0 |
| NT2RP6000060 | 0 | 0 | 0 | 100 | 0 | 0 | 0 |
| NT2RP6000072 | 5.856 | 0 | 0 | 7.789 | 0 | 0 | 0 |
| NT2RP6000077 | 0 | 0 | 0 | 15.492 | 0 | 0 | 0 |
| NT2RP6000078 | 0 | 0 | 0 | 0.867 | 0 | 0 | 1.787 |
| NT2RP6000085 | 0 | 16.809 | 0 | 11.147 | 0 | 0 | 5.745 |
| NT2RP6000086 | 0 | 0 | 0 | 77.250 | 0 | 0 | 0 |
| NT2RP6000100 | 0 | 0 | 0 | 52.773 | 0 | 0 | 0 |
| NT2RP6000109 | 6.922 | 6.941 | 0 | 13.809 | 0 | 0 | 11.863 |
| NT2RP6000110 | 0 | 9.952 | 0 | 6.600 | 0 | 0 | 17.009 |
| NT2RP6000119 | 0 | 0 | 0 | 100 | 0 | 0 | 0 |
| NT2RP6000123 | 1.419 | 2.134 | 0 | 1.887 | 0 | 0 | 2.918 |
| NT2RP6000127 | 0 | 0 | 0 | 100 | 0 | 0 | 0 |
| NT2RP6000130 | 0 | 0 | 0 | 38.584 | 0 | 0 | 0 |
| NT2RP6000140 | 0 | 0 | 0 | 100 | 0 | 0 | 0 |
| NT2RP6000150 | 0 | 0 | 0 | 100 | 0 | 0 | 0 |
| NT2RP7000041 | 0 | 0 | 0 | 100 | 0 | 0 | 0 |
| NT2RP7000069 | 0 | 0 | 0 | 16.237 | 0 | 0 | 4.185 |
| NT2RP7000076 | 0 | 2.215 | 0 | 2.938 | 0 | 0 | 2.271 |
| NT2RP7000086 | 0 | 0 | 0 | 30.820 | 0 | 0 | 0 |
| NT2RP7000109 | 0 | 0 | 0 | 100 | 0 | 0 | 0 |
| NT2RP7000112 | 0 | 0 | 0 | 38.711 | 0 | 0 | 19.954 |
| NT2RP7000173 | 0 | 2.021 | 0 | 13.399 | 0 | 0 | 19.338 |
| NT2RP7000238 | 0 | 16.467 | 0 | 24.959 | 0 | 0 | 1.608 |
| NT2RP7000259 | 0 | 0 | 0 | 32.229 | 0 | 0 | 0 |
| NT2RP7000271 | 0 | 0 | 0 | 7.310 | 0 | 0 | 0 |
| NT2RP7000311 | 0 | 0 | 0 | 100 | 0 | 0 | 0 |
| NT2RP7000359 | 0 | 0 | 0 | 100 | 0 | 0 | 0 |
| NT2RP7000364 | 0 | 0 | 0 | 100 | 0 | 0 | 0 |
| NT2RP7000380 | 0 | 0 | 0 | 100 | 0 | 0 | 0 |
| NT2RP7000391 | 0 | 0 | 0 | 100 | 0 | 0 | 0 |
| NT2RP7000425 | 0 | 0 | 0 | 49.156 | 0 | 0 | 0 |
| NT2RP7000477 | 7.170 | 3.595 | 0 | 14.305 | 0 | 0 | 2.458 |
| NT2RP7000579 | 0 | 0 | 0 | 87.165 | 0 | 0 | 0 |
| NT2RP7000586 | 0 | 19.966 | 0 | 26.480 | 0 | 0 | 0 |
| NT2RP7000600 | 0 | 0 | 0 | 10.681 | 0 | 0 | 2.753 |
| NT2RP7000624 | 0 | 0 | 0 | 10.515 | 0 | 0 | 16.260 |
| NT2RP7000812 | 0 | 0 | 0 | 38.463 | 0 | 0 | 0 |
| NT2RP7000906 | 0 | 2.371 | 0 | 3.144 | 0 | 0 | 3.241 |
| NT2RP7000926 | 0 | 0 | 0 | 100 | 0 | 0 | 0 |
| NT2RP7001074 | 0 | 0 | 0 | 100 | 0 | 0 | 0 |
| NT2RP7001080 | 24.260 | 18.247 | 0 | 32.267 | 0 | 0 | 0 |
| NT2RP7001156 | 0 | 0 | 0 | 78.664 | 0 | 0 | 0 |
| NT2RP7001166 | 2.710 | 2.718 | 0 | 7.210 | 0 | 0 | 1.858 |
| NT2RP7001231 | 0 | 0 | 0 | 9.953 | 0 | 0 | 10.261 |
| NT2RP7001283 | 2.040 | 1.023 | 0 | 2.713 | 0 | 0 | 2.098 |
| NT2RP7001306 | 0 | 0 | 0 | 17.531 | 0 | 0 | 0 |
| NT2RP7001319 | 0 | 0 | 0 | 100 | 0 | 0 | 0 |
| NT2RP7001335 | 0 | 0 | 0 | 25.176 | 0 | 0 | 0 |
| NT2RP7001364 | 0 | 0 | 0 | 70.518 | 0 | 0 | 0 |
| NT2RP7001475 | 0 | 0 | 0 | 42.341 | 0 | 0 | 0 |
| NT2RP7001532 | 0 | 0 | 0 | 100 | 0 | 0 | 0 |
| NT2RP7001591 | 0 | 0 | 0 | 6.266 | 0 | 19.161 | 0 |
| NT2RP7001602 | 0 | 0 | 0 | 100 | 0 | 0 | 0 |
| NT2RP7001678 | 0 | 0 | 0 | 37.485 | 0 | 0 | 0 |
| NT2RP7001856 | 0 | 6.228 | 0 | 8.260 | 0 | 0 | 0 |
| NT2RP7001962 | 0 | 0 | 0 | 12.805 | 0 | 0 | 6.600 |
| NT2RP7002028 | 0 | 0 | 0 | 4.843 | 0 | 0 | 2.496 |
| NT2RP7002064 | 0 | 0 | 0 | 53.332 | 0 | 0 | 0 |
| NT2RP7002151 | 0 | 0 | 0 | 65.987 | 0 | 0 | 34.013 |
| NT2RP7002163 | 0 | 0 | 0 | 100 | 0 | 0 | 0 |
| NT2RP7002243 | 0 | 0 | 0 | 49.240 | 0 | 0 | 50.760 |
| NT2RP7002282 | 0 | 0 | 0 | 37.009 | 0 | 0 | 19.076 |
| NT2RP7002376 | 0 | 0 | 0 | 29.242 | 0 | 0 | 0 |
| NT2RP7002379 | 0 | 0.915 | 0 | 2.426 | 0 | 0 | 3.127 |
| NT2RP7002449 | 0 | 3.671 | 0 | 4.869 | 0 | 0 | 5.019 |
| NT2RP7002450 | 0 | 0 | 0 | 6.432 | 0 | 0 | 0 |
| NT2RP7002478 | 0 | 0 | 0 | 70.259 | 0 | 0 | 0 |
| NT2RP7002554 | 0 | 0 | 0 | 100 | 0 | 0 | 0 |
| NT2RP7002594 | 0 | 0 | 0 | 32.922 | 0 | 0 | 0 |
| NT2RP7002603 | 0 | 0 | 0 | 100 | 0 | 0 | 0 |
| NT2RP7002619 | 0 | 0 | 0 | 2.626 | 0 | 0 | 4.061 |
| NT2RP7002650 | 0 | 0 | 0 | 100 | 0 | 0 | 0 |
| NT2RP7002738 | 0 | 0 | 0 | 27.748 | 0 | 0 | 35.756 |
| NT2RP7002779 | 0 | 0 | 0 | 78.664 | 0 | 0 | 0 |
| NT2RP7002802 | 0 | 6.641 | 0 | 26.423 | 0 | 0 | 9.080 |
| NT2RP7002829 | 0 | 0 | 0 | 21.074 | 0 | 0 | 0 |
| NT2RP7002841 | 0 | 0 | 0 | 100 | 0 | 0 | 0 |
| NT2RP7002875 | 3.344 | 1.677 | 0 | 4.448 | 0 | 0 | 14.902 |
| NT2RP7002906 | 10.161 | 0 | 0 | 6.757 | 0 | 0 | 13.932 |
| NT2RP7002978 | 0 | 0 | 0 | 100 | 0 | 0 | 0 |
| NT2RP7002982 | 0 | 7.064 | 0 | 18.738 | 0 | 0 | 0 |
| NT2RP7003025 | 0 | 0 | 0 | 100 | 0 | 0 | 0 |
| NT2RP7003050 | 0 | 0 | 0 | 100 | 0 | 0 | 0 |
| NT2RP7003055 | 38.242 | 0 | 0 | 25.432 | 0 | 0 | 13.109 |
| NT2RP7003084 | 0 | 0 | 0 | 100 | 0 | 0 | 0 |
| NT2RP7003091 | 0 | 0 | 0 | 100 | 0 | 0 | 0 |
| NT2RP7003107 | 0 | 0 | 0 | 100 | 0 | 0 | 0 |
| NT2RP7003134 | 0 | 0 | 0 | 100 | 0 | 0 | 0 |
| NT2RP7003148 | 0 | 0 | 0 | 100 | 0 | 0 | 0 |
| NT2RP7003203 | 0 | 6.713 | 0 | 4.452 | 0 | 0 | 13.768 |
| NT2RP7003261 | 0 | 0 | 39.095 | 3.274 | 0 | 10.013 | 3.376 |
| NT2RP7003319 | 0 | 1.658 | 0 | 2.199 | 0 | 0 | 5.667 |
| NT2RP7003439 | 0 | 0 | 0 | 100 | 0 | 0 | 0 |
| NT2RP7003511 | 0 | 0 | 0 | 15.928 | 0 | 0 | 8.210 |
| NT2RP7003629 | 0 | 0 | 0 | 27.270 | 0 | 0 | 0 |
| NT2RP7003632 | 5.901 | 0 | 0 | 3.924 | 0 | 12.001 | 2.023 |
| NT2RP7003647 | 0 | 2.139 | 0 | 5.673 | 0 | 0 | 4.386 |
| NT2RP7003680 | 27.035 | 0 | 0 | 35.957 | 0 | 0 | 0 |
| NT2RP7003688 | 0 | 5.367 | 0 | 1.780 | 0 | 5.442 | 8.256 |
| NT2RP7003724 | 0 | 0 | 0 | 22.256 | 0 | 0 | 0 |
| NT2RP7003960 | 0 | 3.011 | 0 | 11.979 | 0 | 0 | 8.233 |
| NT2RP7004027 | 0 | 0 | 0 | 100 | 0 | 0 | 0 |
| NT2RP7004080 | 0 | 0 | 0 | 100 | 0 | 0 | 0 |
| NT2RP7004114 | 0 | 10.133 | 0 | 26.878 | 0 | 0 | 6.927 |
| NT2RP7004123 | 0 | 0 | 0 | 4.625 | 0 | 0 | 0 |
| NT2RP7004173 | 2.768 | 1.388 | 0 | 1.841 | 0 | 0 | 4.744 |
| NT2RP7004196 | 4.748 | 9.524 | 0 | 6.315 | 0 | 0 | 6.511 |
| NT2RP7004204 | 0 | 0 | 0 | 8.219 | 0 | 0 | 2.118 |
| NT2RP7004233 | 0 | 0 | 0 | 28.819 | 0 | 0 | 14.855 |
| NT2RP7004260 | 0 | 0 | 0 | 61.835 | 0 | 0 | 0 |
| NT2RP7004348 | 0 | 4.107 | 0 | 10.893 | 0 | 0 | 16.844 |
| NT2RP7004352 | 0 | 9.259 | 0 | 16.373 | 0 | 0 | 12.659 |
| NT2RP7004358 | 0 | 0 | 0 | 100 | 0 | 0 | 0 |
| NT2RP7004373 | 0 | 0 | 0 | 2.837 | 0 | 0 | 1.462 |
| NT2RP7004396 | 0 | 0 | 0 | 100 | 0 | 0 | 0 |
| NT2RP7004428 | 0 | 0 | 0 | 16.256 | 0 | 0 | 12.569 |
| NT2RP7004481 | 0 | 9.773 | 0 | 38.885 | 0 | 0 | 26.724 |
| NT2RP7004541 | 0 | 0 | 0 | 58.079 | 0 | 0 | 0 |
| NT2RP7004559 | 0 | 0 | 0 | 20.543 | 0 | 0 | 0 |
| NT2RP7004641 | 0 | 0 | 0 | 61.835 | 0 | 0 | 0 |
| NT2RP7004656 | 0 | 0 | 0 | 51.826 | 0 | 0 | 0 |
| NT2RP7004687 | 0 | 0 | 0 | 45.439 | 0 | 0 | 0 |
| NT2RP7004722 | 0 | 0 | 0 | 100 | 0 | 0 | 0 |
| NT2RP7004728 | 0 | 0 | 0 | 7.576 | 0 | 0 | 9.763 |
| NT2RP7004751 | 0 | 0 | 13.468 | 2.256 | 0 | 0 | 0 |
| NT2RP7004766 | 0 | 0 | 0 | 100 | 0 | 0 | 0 |
| NT2RP7004790 | 0 | 0 | 0 | 37.619 | 0 | 0 | 0 |
| NT2RP7004884 | 0 | 0 | 0 | 13.233 | 0 | 0 | 13.642 |
| NT2RP7004911 | 0 | 3.203 | 0 | 4.248 | 0 | 0 | 2.190 |
| NT2RP7004915 | 0 | 13.491 | 0 | 35.784 | 0 | 0 | 0 |
| NT2RP7004925 | 0 | 6.884 | 36.334 | 6.086 | 0 | 0 | 3.137 |
| NT2RP7004946 | 0 | 3.129 | 0 | 8.299 | 0 | 0 | 8.555 |
| NT2RP7004961 | 0 | 0 | 0 | 81.144 | 0 | 0 | 0 |
| NT2RP7004964 | 0 | 0 | 0 | 100 | 0 | 0 | 0 |
| NT2RP7004975 | 0 | 0 | 0 | 100 | 0 | 0 | 0 |
| NT2RP7005118 | 0 | 0 | 0 | 100 | 0 | 0 | 0 |
| NT2RP7005205 | 0 | 0 | 0 | 100 | 0 | 0 | 0 |
| NT2RP7005219 | 0 | 0 | 0 | 62.933 | 0 | 0 | 0 |
| NT2RP7005323 | 0 | 0 | 0 | 100 | 0 | 0 | 0 |
| NT2RP7005468 | 0 | 33.224 | 0 | 44.064 | 0 | 0 | 22.712 |
| NT2RP7005493 | 0 | 0 | 0 | 29.165 | 0 | 0 | 0 |
| NT2RP7005513 | 0 | 0 | 0 | 4.074 | 0 | 0 | 2.100 |
| NT2RP7005529 | 0 | 0 | 0 | 35.588 | 0 | 0 | 0 |
| NT2RP7005629 | 0 | 0 | 0 | 100 | 0 | 0 | 0 |
| NT2RP7005631 | 0 | 0 | 0 | 100 | 0 | 0 | 0 |
| NT2RP7005669 | 0 | 0 | 0 | 100 | 0 | 0 | 0 |
| NT2RP7005675 | 0 | 0 | 0 | 7.009 | 0 | 0 | 7.226 |
| NT2RP7005750 | 0 | 13.005 | 0 | 17.247 | 0 | 0 | 4.445 |
| NT2RP7005846 | 0 | 0 | 0 | 100 | 0 | 0 | 0 |
| NT2RP7006033 | 0 | 0 | 0 | 37.056 | 0 | 0 | 9.550 |
| NT2RP7006075 | 18.990 | 0 | 0 | 6.315 | 0 | 0 | 0 |
| NT2RP7006141 | 0 | 0 | 0 | 1.780 | 0 | 0 | 0 |
| NT2RP7006160 | 0 | 0 | 0 | 100 | 0 | 0 | 0 |
| NT2RP7006162 | 0 | 0 | 0 | 77.250 | 0 | 0 | 0 |
| NT2RP7006188 | 0 | 0 | 0 | 18.824 | 0 | 0 | 6.468 |
| NT2RP7006223 | 0 | 7.678 | 0 | 10.183 | 0 | 0 | 10.498 |
| NT2RP7006263 | 0 | 7.317 | 0 | 9.705 | 0 | 0 | 10.004 |
| NT2RP7006296 | 0 | 0 | 0 | 78.664 | 0 | 0 | 0 |
| NT2RP7006304 | 0 | 0 | 0 | 78.664 | 0 | 0 | 0 |
| NT2RP7006374 | 0 | 0 | 0 | 100 | 0 | 0 | 0 |
| NT2RP7006395 | 0 | 0 | 0 | 82.532 | 0 | 0 | 0 |
| NT2RP7006408 | 0 | 0 | 0 | 100 | 0 | 0 | 0 |
| NT2RP7006457 | 0 | 0 | 0 | 100 | 0 | 0 | 0 |
| NT2RP7006490 | 7.779 | 7.801 | 0 | 5.173 | 0 | 0 | 5.333 |
| NT2RP7006539 | 0 | 0 | 0 | 14.024 | 0 | 0 | 0 |
| NT2RP7006547 | 0 | 0 | 0 | 78.664 | 0 | 0 | 0 |
| NT2RP7006601 | 0 | 0 | 0 | 68.270 | 0 | 0 | 0 |
| NT2RP7006619 | 0 | 0 | 0 | 5.320 | 0 | 0 | 2.742 |
| NT2RP7006621 | 0 | 0 | 0 | 100 | 0 | 0 | 0 |
| NT2RP7006636 | 0 | 0 | 0 | 100 | 0 | 0 | 0 |
| NT2RP7006701 | 18.221 | 0 | 0 | 12.117 | 0 | 0 | 0 |
| NT2RP7006717 | 2.384 | 0 | 0 | 3.171 | 0 | 0 | 5.721 |
| NT2RP7006853 | 0 | 0 | 0 | 100 | 0 | 0 | 0 |
| NT2RP7006886 | 0 | 0 | 0 | 22.923 | 0 | 0 | 11.816 |
| NT2RP7006887 | 0 | 0 | 0 | 100 | 0 | 0 | 0 |
| NT2RP7006980 | 0 | 0 | 0 | 40.647 | 0 | 0 | 0 |
| NT2RP7006986 | 0 | 6.827 | 0 | 9.054 | 0 | 0 | 9.334 |
| NT2RP7006995 | 0 | 0 | 0 | 51.826 | 0 | 0 | 0 |
| NT2RP7007114 | 0 | 0 | 0 | 39.485 | 0 | 0 | 0 |
| NT2RP7007154 | 0 | 0 | 0 | 1.374 | 0 | 0 | 1.417 |
| NT2RP7007177 | 4.935 | 2.475 | 0 | 6.564 | 0 | 0 | 3.383 |
| NT2RP7007221 | 0 | 0 | 0 | 100 | 0 | 0 | 0 |
| NT2RP7007226 | 0 | 0 | 0 | 100 | 0 | 0 | 0 |
| NT2RP7007252 | 0 | 0 | 0 | 33.100 | 0 | 0 | 0 |
| NT2RP7007269 | 0 | 0 | 0 | 56.608 | 0 | 0 | 0 |
| NT2RP7007310 | 0 | 0 | 0 | 8.975 | 0 | 0 | 0 |
| NT2RP7007359 | 0 | 0 | 0 | 100 | 0 | 0 | 0 |
| NT2RP7007381 | 0 | 0 | 0 | 37.796 | 0 | 0 | 0 |
| NT2RP7007387 | 0 | 3.730 | 0 | 9.894 | 0 | 0 | 5.100 |
| NT2RP7007406 | 0 | 0 | 0 | 74.295 | 0 | 0 | 0 |
| NT2RP7007480 | 0 | 0 | 0 | 100 | 0 | 0 | 0 |
| NT2RP7007504 | 0 | 0 | 0 | 100 | 0 | 0 | 0 |
| NT2RP7007530 | 0 | 0 | 0 | 10.568 | 0 | 0 | 2.723 |
| NT2RP7007537 | 4.456 | 2.234 | 0 | 11.854 | 0 | 9.062 | 0 |
| NT2RP7007580 | 0 | 0 | 0 | 57.604 | 0 | 0 | 0 |
| NT2RP7007594 | 0 | 0 | 0 | 100 | 0 | 0 | 0 |
| NT2RP7007610 | 0 | 5.259 | 0 | 13.950 | 0 | 0 | 3.595 |
| NT2RP7007617 | 0 | 28.588 | 0 | 37.916 | 0 | 0 | 0 |
| NT2RP7007643 | 0 | 0 | 0 | 78.664 | 0 | 0 | 0 |
| NT2RP7007766 | 0 | 0 | 0 | 74.295 | 0 | 0 | 0 |
| NT2RP7007842 | 13.902 | 3.485 | 0 | 4.623 | 0 | 0 | 2.383 |
| NT2RP7007925 | 0 | 0 | 0 | 100 | 0 | 0 | 0 |
| NT2RP7007930 | 0 | 0 | 0 | 78.664 | 0 | 0 | 0 |
| NT2RP7007975 | 0 | 4.960 | 0 | 13.156 | 0 | 0 | 1.695 |
| NT2RP7008013 | 0 | 0 | 0 | 100 | 0 | 0 | 0 |
| NT2RP7008015 | 0 | 0 | 0 | 4.863 | 0 | 0 | 1.253 |
| NT2RP7008133 | 0 | 0 | 0 | 100 | 0 | 0 | 0 |
| NT2RP7008137 | 0 | 0 | 0 | 100 | 0 | 0 | 0 |
| NT2RP7008142 | 0 | 0 | 0 | 100 | 0 | 0 | 0 |
| NT2RP7008144 | 0 | 0 | 0 | 100 | 0 | 0 | 0 |
| NT2RP7008161 | 0 | 22.818 | 0 | 15.131 | 0 | 0 | 15.599 |
| NT2RP7008167 | 0 | 1.649 | 0 | 8.750 | 0 | 6.689 | 3.382 |
| NT2RP7008190 | 0 | 0 | 0 | 3.197 | 0 | 0 | 3.296 |
| NT2RP7008315 | 0 | 0 | 0 | 18.087 | 0 | 0 | 0 |
| NT2RP7008360 | 0 | 60.128 | 0 | 39.872 | 0 | 0 | 0 |
| NT2RP7008396 | 0 | 22.370 | 0 | 29.668 | 0 | 0 | 0 |
| NT2RP7008406 | 0 | 0 | 0 | 100 | 0 | 0 | 0 |
| NT2RP7008435 | 0 | 0 | 0 | 100 | 0 | 0 | 0 |
| NT2RP7008441 | 0 | 0 | 0 | 48.519 | 0 | 0 | 0 |
| NT2RP7008454 | 0 | 0 | 0 | 100 | 0 | 0 | 0 |
| NT2RP7008487 | 0 | 0 | 0 | 36.879 | 0 | 0 | 19.009 |
| NT2RP7008543 | 0 | 0 | 0 | 13.261 | 0 | 0 | 3.418 |
| NT2RP7008544 | 0 | 0 | 0 | 100 | 0 | 0 | 0 |
| NT2RP7008550 | 0 | 20.085 | 0 | 79.915 | 0 | 0 | 0 |
| NT2RP7008557 | 0 | 0 | 0 | 100 | 0 | 0 | 0 |
| NT2RP7008623 | 0 | 0 | 0 | 10.373 | 0 | 0 | 0 |
| NT2RP7008657 | 0 | 0 | 0 | 100 | 0 | 0 | 0 |
| NT2RP7008714 | 14.055 | 14.095 | 0 | 9.347 | 0 | 0 | 0 |
| NT2RP7008720 | 0 | 42.988 | 0 | 57.012 | 0 | 0 | 0 |
| NT2RP7008855 | 0 | 0 | 0 | 100 | 0 | 0 | 0 |
| NT2RP7008863 | 0 | 0 | 0 | 100 | 0 | 0 | 0 |
| NT2RP7009012 | 0 | 20.604 | 0 | 27.326 | 0 | 0 | 0 |
| NT2RP7009019 | 0 | 0 | 0 | 100 | 0 | 0 | 0 |
| NT2RP7009030 | 0 | 0 | 0 | 46.373 | 0 | 0 | 35.854 |
| NT2RP7009087 | 0 | 4.415 | 0 | 11.710 | 0 | 17.904 | 6.036 |
| NT2RP7009097 | 0 | 0 | 0 | 4.333 | 0 | 13.250 | 0 |
| NT2RP7009108 | 0 | 0 | 0 | 8.568 | 0 | 0 | 0 |
| NT2RP7009147 | 0 | 2.895 | 0 | 7.679 | 0 | 0 | 1.979 |
| NT2RP7009149 | 0 | 8.696 | 0 | 2.883 | 0 | 0 | 0 |
| NT2RP7009168 | 0 | 0 | 0 | 65.987 | 0 | 0 | 34.013 |
| NT2RP7009215 | 0 | 0 | 0 | 100 | 0 | 0 | 0 |
| NT2RP7009225 | 0 | 15.334 | 0 | 20.337 | 0 | 0 | 3.494 |
| NT2RP7009236 | 0 | 0 | 0 | 100 | 0 | 0 | 0 |
| NT2RP7009253 | 0 | 0 | 0 | 100 | 0 | 0 | 0 |
| NT2RP7009259 | 2.036 | 6.126 | 0 | 9.479 | 0 | 4.141 | 2.094 |
| NT2RP7009267 | 0 | 0 | 0 | 100 | 0 | 0 | 0 |
| NT2RP7009322 | 0 | 0 | 0 | 26.050 | 0 | 0 | 0 |
| NT2RP7009363 | 0 | 0 | 0 | 100 | 0 | 0 | 0 |
| NT2RP7009370 | 0 | 0 | 0 | 78.664 | 0 | 0 | 0 |
| NT2RP7009373 | 14.559 | 0 | 0 | 19.364 | 0 | 0 | 9.981 |
| NT2RP7009394 | 0 | 0 | 0 | 5.829 | 0 | 0 | 6.009 |
| NT2RP7009397 | 0 | 0 | 0 | 100 | 0 | 0 | 0 |
| NT2RP7009429 | 0 | 0 | 0 | 100 | 0 | 0 | 0 |
| NT2RP7009439 | 0 | 0 | 0 | 100 | 0 | 0 | 0 |
| NT2RP7009466 | 0 | 0 | 0 | 74.295 | 0 | 0 | 0 |
| NT2RP7009481 | 0 | 0 | 0 | 29.611 | 0 | 0 | 0 |
| NT2RP7009482 | 0 | 5.053 | 0 | 5.585 | 0 | 0 | 6.333 |
| NT2RP7009498 | 0 | 1.831 | 0 | 2.429 | 0 | 0 | 0 |
| NT2RP7009502 | 0 | 0 | 0 | 100 | 0 | 0 | 0 |
| NT2RP7009507 | 0 | 0 | 0 | 70.676 | 0 | 0 | 0 |
| NT2RP7009577 | 0 | 0 | 0 | 56.608 | 0 | 0 | 0 |
| NT2RP7009743 | 0 | 0 | 0 | 100 | 0 | 0 | 0 |
| NT2RP7009867 | 0 | 0 | 0 | 100 | 0 | 0 | 0 |
| NT2RP7010109 | 0 | 0 | 0 | 75.744 | 0 | 0 | 0 |
| NT2RP7010128 | 0 | 0 | 0 | 100 | 0 | 0 | 0 |
| NT2RP7010235 | 0 | 15.860 | 0 | 84.140 | 0 | 0 | 0 |
| NT2RP7010275 | 0 | 0 | 0 | 100 | 0 | 0 | 0 |
| NT2RP7010521 | 0 | 0 | 0 | 100 | 0 | 0 | 0 |
| NT2RP7010599 | 0 | 0 | 0 | 77.250 | 0 | 0 | 0 |
| NT2RP7010612 | 0 | 0 | 0 | 52.133 | 0 | 0 | 0 |
| NT2RP7010817 | 0 | 0 | 0 | 100 | 0 | 0 | 0 |
| NT2RP7011086 | 0 | 0 | 0 | 100 | 0 | 0 | 0 |
| NT2RP7011132 | 0 | 0 | 0 | 100 | 0 | 0 | 0 |
| NT2RP7011268 | 0 | 0 | 0 | 100 | 0 | 0 | 0 |
| NT2RP7011318 | 0 | 0 | 0 | 56.608 | 0 | 0 | 0 |
| NT2RP7011452 | 0 | 0 | 0 | 100 | 0 | 0 | 0 |
| NT2RP7011570 | 0 | 0 | 0 | 100 | 0 | 0 | 0 |
| NT2RP7011721 | 0 | 0 | 0 | 100 | 0 | 0 | 0 |
| NT2RP7011909 | 0 | 0 | 0 | 0.683 | 93.459 | 0 | 0 |
| NT2RP7012291 | 0 | 0 | 0 | 100 | 0 | 0 | 0 |
| NT2RP7012516 | 0 | 0 | 0 | 100 | 0 | 0 | 0 |
| NT2RP7012776 | 0 | 0 | 0 | 20.256 | 0 | 0 | 0 |
| NT2RP7013002 | 0 | 42.988 | 0 | 57.012 | 0 | 0 | 0 |
| NT2RP7013374 | 0 | 0 | 0 | 100 | 0 | 0 | 0 |
| NT2RP7013499 | 0 | 0 | 0 | 100 | 0 | 0 | 0 |
| NT2RP7013573 | 0 | 13.095 | 0 | 17.367 | 0 | 0 | 8.952 |
| NT2RP7013729 | 0 | 0 | 0 | 100 | 0 | 0 | 0 |
| NT2RP7013764 | 0 | 0 | 0 | 54.725 | 0 | 0 | 0 |
| NT2RP7013795 | 0 | 0 | 0 | 10.432 | 0 | 0 | 0 |
| NT2RP7013999 | 0 | 0 | 0 | 100 | 0 | 0 | 0 |
| NT2RP7014005 | 0 | 21.145 | 0 | 14.022 | 0 | 0 | 0 |
| NT2RP7014178 | 0 | 0 | 0 | 100 | 0 | 0 | 0 |
| NT2RP7014348 | 0 | 0 | 0 | 100 | 0 | 0 | 0 |
| NT2RP7014420 | 0 | 0 | 0 | 100 | 0 | 0 | 0 |
| NT2RP7014583 | 0 | 0 | 0 | 39.434 | 0 | 0 | 0 |
| NT2RP7014721 | 0 | 0 | 0 | 100 | 0 | 0 | 0 |
| NT2RP7014743 | 0 | 0 | 0 | 100 | 0 | 0 | 0 |
| NT2RP7014778 | 0 | 0 | 0 | 100 | 0 | 0 | 0 |
| NT2RP7014906 | 0 | 0 | 0 | 100 | 0 | 0 | 0 |
| NT2RP7014910 | 0 | 6.518 | 0 | 5.763 | 0 | 0 | 2.971 |
| NT2RP7015080 | 0 | 0 | 0 | 100 | 0 | 0 | 0 |
| NT2RP7015431 | 0 | 0 | 0 | 100 | 0 | 0 | 0 |
| NT2RP7015503 | 0 | 0 | 0 | 7.632 | 0 | 0 | 0 |
| NT2RP7015512 | 0 | 0 | 0 | 31.156 | 0 | 0 | 0 |
| NT2RP7015676 | 0 | 0 | 0 | 100 | 0 | 0 | 0 |
| NT2RP7015789 | 0 | 0 | 0 | 100 | 0 | 0 | 0 |
| NT2RP7015996 | 0 | 0 | 0 | 100 | 0 | 0 | 0 |
| NT2RP7016508 | 0 | 0 | 0 | 100 | 0 | 0 | 0 |
| NT2RP7016574 | 0 | 0 | 0 | 53.243 | 0 | 0 | 0 |
| NT2RP7016622 | 0 | 0 | 0 | 100 | 0 | 0 | 0 |
| NT2RP7016911 | 0 | 0 | 0 | 100 | 0 | 0 | 0 |
| NT2RP7017139 | 0 | 0 | 0 | 100 | 0 | 0 | 0 |
| NT2RP7017284 | 0 | 0 | 0 | 100 | 0 | 0 | 0 |
| NT2RP7017365 | 0 | 0 | 0 | 100 | 0 | 0 | 0 |
| NT2RP7017474 | 0 | 0 | 0 | 45.366 | 0 | 0 | 0 |
| NT2RP7017546 | 0 | 0 | 0 | 100 | 0 | 0 | 0 |
| NT2RP7017567 | 0 | 0 | 0 | 100 | 0 | 0 | 0 |
| NT2RP7017795 | 0 | 0 | 0 | 100 | 0 | 0 | 0 |
| NT2RP7017971 | 0 | 0 | 0 | 100 | 0 | 0 | 0 |
| NT2RP7018032 | 0 | 0 | 0 | 100 | 0 | 0 | 0 |
| NT2RP7018126 | 0 | 0 | 0 | 40.584 | 0 | 0 | 0 |
| NT2RP7018197 | 0 | 0 | 0 | 100 | 0 | 0 | 0 |
| NT2RP7018206 | 0 | 0 | 0 | 100 | 0 | 0 | 0 |
| NT2RP7018340 | 0 | 0 | 0 | 100 | 0 | 0 | 0 |
| NT2RP7018586 | 0 | 0 | 0 | 100 | 0 | 0 | 0 |
| NT2RP7018802 | 0 | 0 | 0 | 100 | 0 | 0 | 0 |
| NT2RP7018871 | 0 | 0 | 0 | 100 | 0 | 0 | 0 |
| NT2RP7019064 | 0 | 23.338 | 0 | 30.952 | 0 | 0 | 0 |
| NT2RP7019135 | 0 | 0 | 0 | 70.676 | 0 | 0 | 0 |
| NT2RP7019273 | 0 | 0 | 0 | 100 | 0 | 0 | 0 |
| NT2RP7019367 | 0 | 0 | 0 | 100 | 0 | 0 | 0 |
| NT2RP7019401 | 0 | 0 | 0 | 25.024 | 0 | 0 | 0 |
| NT2RP7019445 | 0 | 0 | 0 | 100 | 0 | 0 | 0 |
| NT2RP7019543 | 0 | 0 | 0 | 50.987 | 0 | 0 | 0 |
| NT2RP7019682 | 0 | 0 | 0 | 100 | 0 | 0 | 0 |
| NT2RP7019835 | 0 | 0 | 0 | 2.512 | 0 | 0 | 0 |
| NT2RP7020112 | 0 | 0 | 0 | 40.874 | 0 | 0 | 0 |
| NT2RP7020123 | 0 | 0 | 0 | 10.250 | 0 | 0 | 0 |
| NT2RP7020343 | 0 | 0 | 0 | 100 | 0 | 0 | 0 |
| NT2RP7020379 | 0 | 0 | 0 | 100 | 0 | 0 | 0 |
| NT2RP8000137 | 0 | 0 | 0 | 100 | 0 | 0 | 0 |
| NT2RP8000296 | 0 | 0 | 0 | 100 | 0 | 0 | 0 |
| NT2RP8000435 | 0 | 24.247 | 0 | 32.158 | 0 | 0 | 0 |
| NT2RP8000483 | 0 | 0 | 0 | 100 | 0 | 0 | 0 |
| NT2RP8000521 | 0 | 0 | 0 | 62.933 | 0 | 0 | 0 |
| NT2RP8000633 | 0 | 0 | 0 | 100 | 0 | 0 | 0 |
| NT2RP8001363 | 0 | 0 | 0 | 100 | 0 | 0 | 0 |
| NT2RP8001407 | 0 | 0 | 0 | 100 | 0 | 0 | 0 |
| NT2RP8001584 | 0 | 0 | 0 | 100 | 0 | 0 | 0 |
| NT2RP8001604 | 0 | 25.218 | 0 | 33.445 | 0 | 0 | 0 |
| NT2RP8001605 | 0 | 0 | 0 | 70.676 | 0 | 0 | 0 |
| NT2RP8003490 | 0 | 0 | 0 | 100 | 0 | 0 | 0 |
| NT2RP8003657 | 0 | 0 | 0 | 100 | 0 | 0 | 0 |
| NT2RP8003787 | 0 | 0 | 0 | 100 | 0 | 0 | 0 |
| NT2RP8004306 | 0 | 0 | 0 | 100 | 0 | 0 | 0 |
| NT2RP8005546 | 0 | 0 | 0 | 59.103 | 0 | 0 | 0 |
| NT2RP8006452 | 0 | 0 | 0 | 100 | 0 | 0 | 0 |
| NT2RP8006521 | 0 | 0 | 0 | 100 | 0 | 0 | 0 |
| NT2RP8007416 | 0 | 0 | 0 | 100 | 0 | 0 | 0 |
| NT2RP8007503 | 0 | 0 | 0 | 100 | 0 | 0 | 0 |
| NT2RP8007715 | 0 | 0 | 0 | 30.282 | 0 | 0 | 0 |
| NT2RP8007920 | 0 | 0 | 0 | 9.535 | 0 | 0 | 31.946 |
| NT2RP8008057 | 0 | 0 | 0 | 68.270 | 0 | 0 | 0 |
| NT2RP8009248 | 0 | 0 | 0 | 100 | 0 | 0 | 0 |
| NTONG1000006 | 0 | 0 | 0 | 0 | 0 | 100 | 0 |
| NTONG1000033 | 0 | 6.679 | 0 | 0 | 0 | 9.030 | 0 |
| NTONG1000047 | 0 | 0 | 0 | 0 | 0 | 44.281 | 0 |
| NTONG1000052 | 0 | 0 | 0 | 0 | 0 | 52.799 | 0 |
| NTONG1000075 | 0 | 0 | 0 | 0 | 0 | 49.103 | 0 |
| NTONG1000087 | 0 | 0 | 0 | 0 | 0 | 100 | 0 |
| NTONG1000098 | 0 | 2.316 | 0 | 3.071 | 0 | 4.695 | 4.749 |
| NTONG1000123 | 0 | 0 | 0 | 0 | 0 | 68.634 | 0 |
| NTONG1000130 | 0 | 0 | 0 | 0 | 0 | 86.807 | 0 |
| NTONG1000161 | 0 | 0 | 0 | 0 | 0 | 100 | 0 |
| NTONG1000172 | 0 | 0 | 0 | 0 | 0 | 100 | 0 |
| NTONG1000182 | 0 | 2.250 | 0 | 0 | 0 | 27.372 | 0 |
| NTONG1000213 | 0 | 0 | 0 | 0 | 0 | 100 | 0 |
| NTONG1000214 | 0 | 0 | 0 | 8.919 | 0 | 81.823 | 0 |
| NTONG1000246 | 0 | 0 | 0 | 0 | 0 | 11.524 | 0 |
| NTONG1000247 | 0 | 0 | 0 | 0 | 0 | 100 | 0 |
| NTONG1000257 | 0 | 0 | 0 | 0 | 0 | 8.111 | 0 |
| NTONG1000264 | 3.532 | 1.771 | 0 | 2.349 | 0 | 7.183 | 15.741 |
| NTONG2000040 | 0 | 0 | 0 | 0 | 0 | 100 | 0 |
| NTONG2000107 | 0 | 0 | 0 | 0 | 0 | 78.591 | 0 |
| NTONG2000231 | 0 | 0 | 0 | 0 | 0 | 100 | 0 |
| NTONG2000265 | 0 | 0 | 0 | 2.055 | 0 | 6.283 | 3.177 |
| NTONG2000334 | 0 | 0 | 0 | 0 | 0 | 15.068 | 7.620 |
| NTONG2000363 | 0 | 0 | 0 | 0 | 0 | 100 | 0 |
| NTONG2000413 | 0 | 0 | 0 | 0 | 0 | 79.538 | 13.407 |
| NTONG2000492 | 0 | 0 | 0 | 0 | 0 | 100 | 0 |
| NTONG2000499 | 0 | 0 | 0 | 0 | 0 | 68.022 | 0 |
| NTONG2000531 | 0 | 0 | 0 | 0 | 0 | 100 | 0 |
| NTONG2000583 | 0 | 0 | 0 | 0 | 0 | 100 | 0 |
| NTONG2000800 | 0 | 0 | 0 | 0 | 0 | 15.610 | 0 |
| NTONG2000858 | 0 | 0 | 0 | 0 | 0 | 21.285 | 0 |
| NTONG2000876 | 0 | 0 | 0 | 0 | 0 | 10.262 | 1.730 |
| NTONG2000878 | 0 | 2.146 | 0 | 0 | 0 | 8.703 | 0 |
| NTONG2000966 | 0 | 0 | 0 | 0 | 0 | 100 | 0 |
| NTONG2000977 | 0 | 0 | 0 | 0 | 0 | 7.245 | 2.442 |
| NTONG2000985 | 0 | 0 | 53.648 | 0 | 0 | 13.740 | 2.316 |
| NTONG2001014 | 0 | 0 | 0 | 0 | 0 | 100 | 0 |
| NTONG2001079 | 0 | 0 | 0 | 0 | 0 | 85.575 | 14.425 |
| NTONG2001137 | 0 | 0 | 0 | 0 | 0 | 8.434 | 0 |
| NTONG2001157 | 0 | 0 | 0 | 0 | 0 | 100 | 0 |
| NTONG2001173 | 0 | 0 | 0 | 0 | 0 | 100 | 0 |
| NTONG2001220 | 0 | 0 | 0 | 0 | 0 | 42.409 | 0 |
| NTONG2001222 | 0 | 0 | 0 | 0 | 0 | 30.557 | 0 |
| NTONG2001362 | 0 | 0 | 0 | 0 | 0 | 25.727 | 0 |
| NTONG2001428 | 0 | 0 | 0 | 0 | 0 | 77.689 | 0 |
| NTONG2001532 | 0 | 0 | 0 | 0 | 0 | 100 | 0 |
| NTONG2001550 | 0 | 0 | 0 | 0 | 0 | 25.651 | 0 |
| NTONG2001567 | 0 | 0 | 0 | 0 | 0 | 9.039 | 0 |
| NTONG2001587 | 0 | 0 | 0 | 0 | 0 | 17.552 | 0 |
| NTONG2001612 | 0 | 2.040 | 0 | 2.705 | 0 | 8.273 | 4.184 |
| NTONG2001619 | 0 | 0 | 0 | 0 | 0 | 100 | 0 |
| NTONG2001642 | 0 | 0 | 0 | 0 | 0 | 100 | 0 |
| NTONG2001657 | 0 | 0 | 0 | 0 | 0 | 48.867 | 0 |
| NTONG2001676 | 0 | 0 | 0 | 17.193 | 0 | 52.573 | 0 |
| NTONG2001762 | 0 | 0 | 0 | 0 | 0 | 13.290 | 11.201 |
| NTONG2002278 | 0 | 0 | 0 | 0 | 0 | 100 | 0 |
| NTONG2002582 | 0 | 0 | 0 | 0 | 0 | 27.719 | 0 |
| NTONG2002807 | 0 | 0 | 0 | 0 | 0 | 86.921 | 0 |
| NTONG2002948 | 0 | 0 | 0 | 0 | 0 | 32.729 | 0 |
| NTONG2002970 | 0 | 0 | 0 | 0 | 0 | 100 | 0 |
| NTONG2002985 | 0 | 0 | 0 | 0 | 0 | 88.053 | 0 |
| NTONG2003158 | 0 | 0 | 0 | 0 | 0 | 100 | 0 |
| NTONG2003210 | 0 | 0 | 0 | 0 | 0 | 100 | 0 |
| NTONG2003316 | 0 | 0 | 0 | 0 | 0 | 100 | 0 |
| NTONG2003409 | 0 | 0 | 0 | 0 | 0 | 100 | 0 |
| NTONG2003454 | 0 | 0 | 0 | 0 | 0 | 21.971 | 0 |
| NTONG2003515 | 0 | 0 | 0 | 0 | 0 | 72.123 | 0 |
| NTONG2003805 | 0 | 0 | 0 | 0 | 0 | 100 | 0 |
| NTONG2003839 | 0 | 9.529 | 0 | 0 | 0 | 19.323 | 0 |
| NTONG2003852 | 0 | 0 | 0 | 0 | 0 | 49.507 | 0 |
| NTONG2003897 | 0 | 0 | 0 | 0 | 0 | 100 | 0 |
| NTONG2003928 | 0 | 0 | 0 | 0 | 0 | 42.932 | 0 |
| NTONG2004095 | 0 | 0 | 0 | 0 | 0 | 67.666 | 0 |
| NTONG2004308 | 0 | 0 | 0 | 0 | 0 | 86.807 | 0 |
| NTONG2004521 | 0 | 0 | 0 | 0 | 0 | 100 | 0 |
| NTONG2004614 | 0 | 0 | 0 | 0 | 0 | 43.643 | 0 |
| NTONG2004690 | 0 | 0 | 0 | 0 | 0 | 100 | 0 |
| NTONG2004806 | 0 | 0 | 0 | 0 | 0 | 51.123 | 0 |
| NTONG2004829 | 0 | 0 | 0 | 0 | 0 | 100 | 0 |
| NTONG2004844 | 0 | 10 | 0 | 0 | 0 | 11.944 | 0 |
| NTONG2004918 | 0 | 0 | 0 | 0 | 0 | 47.726 | 0 |
| NTONG2004991 | 0 | 0 | 0 | 0 | 0 | 100 | 0 |
| NTONG2005062 | 0 | 0 | 0 | 0 | 0 | 100 | 0 |
| NTONG2005086 | 0 | 0 | 0 | 0 | 0 | 100 | 0 |
| NTONG2005137 | 0 | 0 | 0 | 0 | 0 | 100 | 0 |
| NTONG2005153 | 0 | 0 | 0 | 0 | 0 | 58.314 | 0 |
| NTONG2005265 | 0 | 0 | 0 | 0 | 0 | 100 | 0 |
| NTONG2005277 | 0 | 0 | 0 | 0 | 0 | 100 | 0 |
| NTONG2005363 | 0 | 0 | 0 | 0 | 0 | 86.071 | 0 |
| NTONG2005373 | 0 | 0 | 0 | 0 | 0 | 86.864 | 0 |
| NTONG2005391 | 0 | 4.236 | 0 | 11.237 | 0 | 17.181 | 2.896 |
| NTONG2005480 | 0 | 0 | 0 | 0 | 0 | 100 | 0 |
| NTONG2005497 | 0 | 0 | 0 | 0 | 90.363 | 2.020 | 0 |
| NTONG2005520 | 0 | 6.048 | 0 | 0 | 0 | 4.906 | 0 |
| NTONG2005577 | 0 | 0 | 0 | 0 | 0 | 100 | 0 |
| NTONG2005657 | 0 | 0 | 0 | 0 | 0 | 100 | 0 |
| NTONG2005801 | 0 | 0 | 0 | 0 | 0 | 26.260 | 0 |
| NTONG2005822 | 0 | 0 | 0 | 0 | 0 | 67.622 | 0 |
| NTONG2005897 | 0 | 0 | 0 | 0 | 0 | 50.588 | 0 |
| NTONG2005969 | 0 | 0 | 0 | 15.484 | 0 | 23.675 | 0 |
| NTONG2006099 | 0 | 0 | 0 | 0 | 0 | 100 | 0 |
| NTONG2006187 | 0 | 0 | 0 | 0 | 0 | 100 | 0 |
| NTONG2006301 | 0 | 0 | 0 | 0 | 0 | 40.505 | 0 |
| NTONG2006324 | 0 | 0 | 0 | 0 | 0 | 100 | 0 |
| NTONG2006354 | 0 | 0 | 0 | 0 | 0 | 100 | 0 |
| NTONG2006440 | 0 | 0 | 0 | 0 | 0 | 100 | 0 |
| NTONG2006484 | 0 | 0 | 0 | 0 | 0 | 31.857 | 0 |
| NTONG2006501 | 0 | 0 | 0 | 0 | 0 | 100 | 0 |
| NTONG2006646 | 0 | 0 | 0 | 0 | 0 | 100 | 0 |
| NTONG2006709 | 0 | 16.469 | 0 | 0 | 0 | 66.789 | 0 |
| NTONG2006783 | 0 | 0 | 0 | 0 | 0 | 20.865 | 0 |
| NTONG2007020 | 0 | 0 | 0 | 0 | 0 | 100 | 0 |
| NTONG2007028 | 0 | 0 | 0 | 0 | 0 | 88.053 | 0 |
| NTONG2007034 | 0 | 0 | 0 | 0 | 0 | 52.247 | 0 |
| NTONG2007249 | 0 | 0 | 0 | 0 | 0 | 100 | 0 |
| NTONG2007517 | 0 | 0 | 0 | 0 | 0 | 100 | 0 |
| NTONG2007522 | 0 | 0 | 0 | 0 | 0 | 100 | 0 |
| NTONG2007658 | 0 | 0 | 0 | 0 | 0 | 14.749 | 0 |
| NTONG2007693 | 0 | 0 | 0 | 0 | 0 | 51.858 | 0 |
| NTONG2007756 | 0 | 0 | 0 | 0 | 0 | 49.744 | 0 |
| NTONG2008088 | 0 | 0 | 0 | 0 | 0 | 75.309 | 0 |
| NTONG2008093 | 0 | 0 | 0 | 0 | 0 | 100 | 0 |
| NTONG2008143 | 0 | 0 | 0 | 0 | 0 | 80.075 | 0 |
| NTONG2008365 | 0 | 0 | 0 | 0 | 0 | 100 | 0 |
| NTONG2008483 | 0 | 4.356 | 0 | 0 | 0 | 17.664 | 1.985 |
| NTONG2008522 | 0 | 0 | 0 | 0 | 0 | 57.763 | 0 |
| NTONG2008672 | 0 | 0 | 0 | 0 | 0 | 100 | 0 |
| NTONG2008862 | 0 | 0 | 0 | 0 | 0 | 100 | 0 |
| NTONG2008939 | 0 | 0 | 0 | 0 | 0 | 100 | 0 |
| NTONG2008944 | 0 | 0 | 0 | 14.075 | 0 | 21.519 | 3.627 |
| NTONG2009060 | 0 | 0 | 0 | 0 | 0 | 20.536 | 0 |
| NTONG2009068 | 0 | 0 | 0 | 0 | 0 | 100 | 0 |
| NTONG2009229 | 0 | 0 | 0 | 0 | 0 | 100 | 0 |
| NTONG2009233 | 0 | 0 | 0 | 0 | 0 | 100 | 0 |
| NTONG2009468 | 0 | 0 | 0 | 0 | 0 | 69.996 | 0 |
| NTONG2009576 | 0 | 0 | 0 | 0 | 0 | 52.366 | 0 |
| OCBBF1000016 | 0 | 0 | 0 | 0 | 0 | 0 | 100 |
| OCBBF1000030 | 0 | 0 | 0 | 0 | 0 | 0 | 54.907 |
| OCBBF1000042 | 0 | 0 | 0 | 0 | 0 | 0 | 100 |
| OCBBF1000049 | 0 | 9.030 | 0 | 0 | 0 | 0 | 49.386 |
| OCBBF1000054 | 0 | 0 | 0 | 0 | 0 | 0 | 100 |
| OCBBF1000067 | 0 | 0 | 0 | 0 | 0 | 0 | 22.706 |
| OCBBF1000080 | 0 | 0 | 0 | 0 | 0 | 0 | 23.753 |
| OCBBF1000085 | 0 | 0 | 0 | 0 | 0 | 0 | 100 |
| OCBBF1000086 | 0 | 0 | 0 | 1.845 | 0 | 0 | 1.902 |
| OCBBF1000091 | 0 | 0 | 0 | 0 | 0 | 0 | 100 |
| OCBBF1000104 | 0 | 0 | 0 | 24.822 | 0 | 0 | 25.588 |
| OCBBF1000114 | 0 | 0 | 0 | 0 | 0 | 0 | 3.167 |
| OCBBF1000118 | 0 | 0 | 0 | 0 | 0 | 0 | 100 |
| OCBBF1000119 | 0 | 0 | 0 | 0 | 0 | 0 | 100 |
| OCBBF1000122 | 0 | 0 | 0 | 0 | 0 | 0 | 100 |
| OCBBF1000130 | 0 | 0 | 0 | 0 | 0 | 0 | 100 |
| OCBBF1000138 | 0 | 0 | 0 | 0 | 0 | 0 | 100 |
| OCBBF1000145 | 0 | 0 | 0 | 0 | 0 | 0 | 52.835 |
| OCBBF1000146 | 6.252 | 1.568 | 0 | 6.237 | 0 | 0 | 10.716 |
| OCBBF1000153 | 0 | 0 | 0 | 0 | 0 | 0 | 84.325 |
| OCBBF1000175 | 0 | 13.090 | 0 | 0 | 0 | 0 | 17.896 |
| OCBBF1000185 | 0 | 7.650 | 0 | 20.293 | 0 | 0 | 31.379 |
| OCBBF1000188 | 3.774 | 1.892 | 0 | 0 | 0 | 0 | 3.881 |
| OCBBF1000192 | 0 | 0 | 0 | 0 | 0 | 0 | 100 |
| OCBBF1000254 | 0 | 0 | 0 | 0 | 0 | 0 | 55.216 |
| OCBBF1000315 | 0 | 0 | 0 | 0 | 0 | 0 | 21.738 |
| OCBBF2000013 | 0 | 14.144 | 0 | 4.690 | 0 | 0 | 12.086 |
| OCBBF2000015 | 0 | 2.875 | 0 | 11.441 | 0 | 0 | 13.760 |
| OCBBF2000126 | 0 | 0 | 0 | 9.367 | 0 | 0 | 4.828 |
| OCBBF2000178 | 11.854 | 1.981 | 0 | 2.628 | 0 | 0 | 31.152 |
| OCBBF2000231 | 0 | 0 | 0 | 9.441 | 0 | 0 | 29.197 |
| OCBBF2000277 | 0 | 26.701 | 0 | 0 | 0 | 0 | 18.253 |
| OCBBF2000287 | 0 | 0 | 0 | 0 | 0 | 0 | 26.038 |
| OCBBF2000323 | 0 | 0 | 0 | 0 | 0 | 0 | 18.850 |
| OCBBF2000452 | 0 | 0 | 0 | 0 | 0 | 0 | 100 |
| OCBBF2000467 | 0 | 0 | 0 | 0 | 0 | 14.865 | 22.551 |
| OCBBF2000522 | 0 | 4.595 | 0 | 0 | 0 | 18.636 | 3.141 |
| OCBBF2000677 | 0 | 0 | 0 | 0 | 0 | 0 | 20.236 |
| OCBBF2000703 | 0 | 0 | 0 | 0 | 0 | 0 | 100 |
| OCBBF2000712 | 0 | 0 | 0 | 0 | 0 | 0 | 2.348 |
| OCBBF2000719 | 0 | 0 | 0 | 0 | 0 | 0 | 100 |
| OCBBF2000824 | 0 | 0 | 0 | 0 | 0 | 0 | 14.930 |
| OCBBF2000831 | 0 | 0 | 0 | 0 | 0 | 0 | 47.849 |
| OCBBF2000846 | 0 | 0 | 0 | 0 | 0 | 0 | 0.909 |
| OCBBF2000904 | 29.296 | 0 | 0 | 9.741 | 0 | 0 | 10.042 |
| OCBBF2000982 | 0 | 0 | 25.094 | 0 | 0 | 0 | 6.500 |
| OCBBF2000986 | 0 | 3.502 | 0 | 2.323 | 0 | 0 | 8.380 |
| OCBBF2000998 | 0 | 0 | 0 | 0 | 0 | 0 | 100 |
| OCBBF2001075 | 0 | 0 | 0 | 0 | 0 | 0 | 100 |
| OCBBF2001101 | 0 | 0 | 0 | 0 | 0 | 0 | 100 |
| OCBBF2001124 | 0 | 0 | 0 | 0 | 0 | 0 | 100 |
| OCBBF2001140 | 0 | 0 | 0 | 0 | 0 | 0 | 7.024 |
| OCBBF2001166 | 0 | 0 | 0 | 0 | 0 | 0 | 13.617 |
| OCBBF2001176 | 0 | 0 | 0 | 0 | 0 | 0 | 100 |
| OCBBF2001186 | 0 | 0 | 0 | 0 | 0 | 0 | 43.982 |
| OCBBF2001196 | 0 | 0 | 0 | 0 | 0 | 0 | 19.673 |
| OCBBF2001210 | 0 | 0 | 10 | 0 | 0 | 0 | 27.026 |
| OCBBF2001252 | 0 | 0 | 0 | 0 | 0 | 0 | 40.027 |
| OCBBF2001307 | 0 | 0 | 0 | 0 | 0 | 0 | 2.066 |
| OCBBF2001323 | 0 | 0 | 0 | 20.724 | 0 | 0 | 21.364 |
| OCBBF2001389 | 0 | 0 | 0 | 0 | 0 | 0 | 100 |
| OCBBF2001402 | 0 | 0 | 0 | 0 | 0 | 0 | 40.207 |
| OCBBF2001408 | 0 | 0 | 0 | 0 | 0 | 0 | 47.976 |
| OCBBF2001416 | 0 | 0 | 0 | 0 | 0 | 0 | 51.849 |
| OCBBF2001473 | 0 | 0 | 0 | 0 | 0 | 0 | 100 |
| OCBBF2001492 | 2.487 | 0 | 0 | 3.307 | 0 | 0 | 8.524 |
| OCBBF2001494 | 0 | 0 | 0 | 0 | 0 | 0 | 26.272 |
| OCBBF2001527 | 0 | 0 | 0 | 0 | 0 | 0 | 100 |
| OCBBF2001528 | 0 | 0 | 0 | 0 | 0 | 0 | 41.188 |
| OCBBF2001586 | 12.994 | 0 | 0 | 8.642 | 0 | 26.425 | 4.454 |
| OCBBF2001639 | 0 | 0 | 0 | 0 | 0 | 0 | 5.842 |
| OCBBF2001681 | 0 | 0 | 0 | 0 | 0 | 0 | 100 |
| OCBBF2001687 | 0 | 0 | 0 | 0 | 0 | 0 | 7.169 |
| OCBBF2001706 | 0 | 0 | 0 | 0 | 0 | 0 | 59.836 |
| OCBBF2001749 | 0 | 0 | 0 | 0 | 0 | 0 | 40.207 |
| OCBBF2001794 | 0 | 0 | 0 | 0 | 0 | 34.843 | 11.746 |
| OCBBF2001910 | 0 | 0 | 0 | 0 | 0 | 0 | 100 |
| OCBBF2001931 | 0 | 0 | 0 | 0 | 0 | 0 | 20.802 |
| OCBBF2001961 | 0 | 0 | 0 | 0 | 0 | 0 | 51.019 |
| OCBBF2001983 | 0 | 0 | 0 | 0 | 0 | 0 | 8.974 |
| OCBBF2002015 | 0 | 0 | 0 | 0 | 0 | 0 | 17.182 |
| OCBBF2002083 | 0 | 0 | 0 | 0 | 0 | 0 | 27.302 |
| OCBBF2002086 | 0 | 5.855 | 0 | 7.765 | 0 | 0 | 4.002 |
| OCBBF2002124 | 0 | 0 | 0 | 0 | 0 | 0 | 100 |
| OCBBF2002161 | 0 | 0 | 0 | 0 | 0 | 0 | 100 |
| OCBBF2002290 | 0 | 0 | 0 | 0 | 0 | 0 | 12.529 |
| OCBBF2002357 | 0 | 0 | 0 | 0 | 0 | 0 | 100 |
| OCBBF2002376 | 0 | 0 | 0 | 0 | 0 | 0 | 18.916 |
| OCBBF2002615 | 0 | 0 | 0 | 0 | 0 | 0 | 100 |
| OCBBF2002626 | 3.060 | 1.535 | 0 | 0 | 0 | 0 | 4.196 |
| OCBBF2002656 | 0 | 0 | 0 | 0 | 0 | 0 | 6.165 |
| OCBBF2002663 | 0 | 0 | 0 | 0 | 0 | 0 | 40.207 |
| OCBBF2002805 | 0 | 0 | 0 | 0 | 0 | 0 | 100 |
| OCBBF2002865 | 0 | 0 | 0 | 0 | 0 | 0 | 65.522 |
| OCBBF2002920 | 0 | 0 | 0 | 0 | 0 | 0 | 100 |
| OCBBF2002980 | 0 | 0 | 0 | 0 | 0 | 0 | 54.907 |
| OCBBF2003028 | 0 | 0 | 0 | 0 | 0 | 0 | 100 |
| OCBBF2003052 | 0 | 0 | 0 | 0 | 0 | 0 | 14.156 |
| OCBBF2003091 | 0 | 0 | 0 | 0 | 0 | 0 | 100 |
| OCBBF2003246 | 0 | 0 | 0 | 0 | 0 | 0 | 24.315 |
| OCBBF2003327 | 0 | 0 | 0 | 0 | 0 | 0 | 100 |
| OCBBF2003518 | 0 | 1.873 | 0 | 0 | 0 | 0 | 1.281 |
| OCBBF2003543 | 0 | 0 | 0 | 0 | 0 | 0 | 59.836 |
| OCBBF2003593 | 0 | 0 | 0 | 0 | 0 | 0 | 55.403 |
| OCBBF2003744 | 0 | 0 | 0 | 0 | 0 | 0 | 52.585 |
| OCBBF2003819 | 0 | 0 | 0 | 0 | 0 | 0 | 26.175 |
| OCBBF2003925 | 0 | 0 | 0 | 4.016 | 0 | 0 | 1.035 |
| OCBBF2004038 | 0 | 0 | 0 | 0 | 0 | 0 | 33.259 |
| OCBBF2004104 | 0 | 0 | 0 | 0 | 0 | 0 | 22.118 |
| OCBBF2004147 | 0 | 0 | 0 | 0 | 0 | 0 | 100 |
| OCBBF2004168 | 0 | 0 | 0 | 0 | 0 | 0 | 100 |
| OCBBF2004235 | 0 | 0 | 0 | 0 | 0 | 0 | 14.817 |
| OCBBF2004259 | 0 | 0 | 0 | 0 | 0 | 0 | 52.835 |
| OCBBF2004273 | 0 | 4.887 | 0 | 0 | 0 | 0 | 3.341 |
| OCBBF2004385 | 0 | 0 | 0 | 0 | 0 | 0 | 100 |
| OCBBF2004418 | 0 | 0 | 0 | 0 | 0 | 0 | 17.655 |
| OCBBF2004478 | 0 | 0 | 0 | 0 | 0 | 0 | 100 |
| OCBBF2004482 | 0 | 0 | 0 | 0 | 0 | 0 | 100 |
| OCBBF2004533 | 0 | 0 | 0 | 0 | 0 | 0 | 25.025 |
| OCBBF2004567 | 0 | 0 | 0 | 0 | 0 | 0 | 100 |
| OCBBF2004612 | 0 | 0 | 0 | 0 | 0 | 0 | 100 |
| OCBBF2004647 | 0 | 0 | 0 | 0 | 0 | 0 | 100 |
| OCBBF2004669 | 0 | 0 | 0 | 0 | 0 | 0 | 54.907 |
| OCBBF2004757 | 0 | 4.504 | 0 | 0 | 0 | 0 | 12.315 |
| OCBBF2004826 | 0 | 0 | 0 | 0 | 0 | 0 | 100 |
| OCBBF2004866 | 0 | 0 | 0 | 0 | 0 | 0 | 100 |
| OCBBF2004883 | 0 | 0 | 0 | 0 | 0 | 0 | 100 |
| OCBBF2004889 | 0 | 0 | 0 | 0 | 0 | 0 | 55.403 |
| OCBBF2004930 | 0 | 0 | 0 | 0 | 0 | 0 | 12.893 |
| OCBBF2004984 | 0 | 0 | 0 | 0 | 0 | 0 | 29.587 |
| OCBBF2005066 | 0 | 0 | 0 | 0 | 0 | 0 | 100 |
| OCBBF2005077 | 0 | 0 | 0 | 0 | 0 | 0 | 100 |
| OCBBF2005161 | 0 | 0 | 0 | 0 | 0 | 0 | 54.907 |
| OCBBF2005343 | 0 | 0 | 0 | 0 | 0 | 0 | 100 |
| OCBBF2005349 | 0 | 0 | 0 | 0 | 0 | 0 | 100 |
| OCBBF2005373 | 0 | 0 | 0 | 0 | 0 | 0 | 100 |
| OCBBF2005420 | 0 | 0 | 0 | 0 | 0 | 0 | 78.844 |
| OCBBF2005428 | 0 | 0 | 0 | 0 | 0 | 0 | 4.095 |
| OCBBF2005433 | 0 | 0 | 0 | 0 | 0 | 0 | 100 |
| OCBBF2005476 | 0 | 8.495 | 0 | 0 | 0 | 0 | 5.808 |
| OCBBF2005546 | 0 | 0 | 0 | 0 | 0 | 0 | 54.907 |
| OCBBF2005843 | 0 | 0 | 0 | 0 | 0 | 0 | 100 |
| OCBBF2005901 | 0 | 0 | 0 | 0 | 0 | 0 | 100 |
| OCBBF2005956 | 0 | 10.318 | 0 | 0 | 0 | 0 | 3.527 |
| OCBBF2006005 | 0 | 0 | 0 | 0 | 0 | 0 | 5.922 |
| OCBBF2006030 | 0 | 0 | 0 | 0 | 0 | 0 | 100 |
| OCBBF2006058 | 0 | 0 | 0 | 0 | 0 | 0 | 100 |
| OCBBF2006113 | 0 | 0 | 0 | 0 | 0 | 0 | 32.696 |
| OCBBF2006148 | 0 | 0 | 0 | 0 | 0 | 0 | 16.826 |
| OCBBF2006151 | 0 | 0 | 0 | 0 | 0 | 0 | 7.162 |
| OCBBF2006154 | 0 | 0 | 0 | 0 | 0 | 0 | 100 |
| OCBBF2006172 | 0 | 0 | 0 | 0 | 0 | 0 | 13.027 |
| OCBBF2006214 | 0 | 0 | 0 | 0 | 0 | 0 | 100 |
| OCBBF2006241 | 0 | 0 | 0 | 0 | 0 | 0 | 100 |
| OCBBF2006313 | 0 | 0 | 0 | 13.761 | 0 | 0 | 7.093 |
| OCBBF2006388 | 0 | 0 | 0 | 0 | 0 | 0 | 100 |
| OCBBF2006438 | 0 | 0 | 0 | 0 | 0 | 0 | 100 |
| OCBBF2006567 | 0 | 0 | 0 | 0 | 0 | 0 | 100 |
| OCBBF2006624 | 0 | 0 | 0 | 0 | 0 | 0 | 100 |
| OCBBF2006639 | 0 | 0 | 0 | 0 | 0 | 0 | 100 |
| OCBBF2006660 | 0 | 0 | 0 | 0 | 0 | 0 | 18.751 |
| OCBBF2006764 | 0 | 0 | 0 | 0 | 0 | 0 | 19.063 |
| OCBBF2006849 | 0 | 0 | 0 | 0 | 0 | 14.895 | 7.532 |
| OCBBF2006859 | 0 | 13.615 | 0 | 0 | 0 | 0 | 9.307 |
| OCBBF2006972 | 0 | 0 | 0 | 0 | 0 | 0 | 55.403 |
| OCBBF2006987 | 0 | 0 | 0 | 62.306 | 0 | 0 | 16.058 |
| OCBBF2007028 | 0 | 2.509 | 0 | 0 | 0 | 0 | 15.437 |
| OCBBF2007039 | 0 | 0 | 0 | 0 | 0 | 0 | 100 |
| OCBBF2007051 | 0 | 0 | 0 | 0 | 0 | 0 | 54.907 |
| OCBBF2007068 | 0 | 0 | 0 | 0 | 0 | 0 | 54.907 |
| OCBBF2007114 | 0 | 0 | 0 | 0 | 0 | 0 | 39.836 |
| OCBBF2007121 | 0 | 0 | 0 | 0 | 0 | 0 | 52.585 |
| OCBBF2007184 | 0 | 0 | 0 | 0 | 0 | 0 | 100 |
| OCBBF2007194 | 0 | 0 | 0 | 0 | 0 | 0 | 100 |
| OCBBF2007196 | 0 | 0 | 0 | 0 | 0 | 0 | 66.858 |
| OCBBF2007224 | 0 | 0 | 0 | 0 | 0 | 0 | 11.730 |
| OCBBF2007238 | 0 | 0 | 0 | 0 | 0 | 0 | 100 |
| OCBBF2007354 | 0 | 3.651 | 0 | 0 | 0 | 0 | 1.248 |
| OCBBF2007414 | 0 | 0 | 0 | 0 | 0 | 0 | 59.836 |
| OCBBF2007415 | 2.902 | 0 | 0 | 1.930 | 0 | 0 | 1.990 |
| OCBBF2007428 | 0 | 0 | 0 | 0 | 0 | 0 | 100 |
| OCBBF2007478 | 0 | 0 | 0 | 0 | 0 | 0 | 100 |
| OCBBF2007485 | 0 | 0 | 0 | 0 | 0 | 0 | 100 |
| OCBBF2007520 | 0 | 0 | 0 | 0 | 0 | 0 | 100 |
| OCBBF2007555 | 0 | 0 | 0 | 0 | 0 | 0 | 100 |
| OCBBF2007610 | 0 | 0 | 0 | 0 | 0 | 0 | 100 |
| OCBBF2007622 | 0 | 0 | 0 | 0 | 0 | 0 | 4.561 |
| OCBBF2007756 | 0 | 0 | 0 | 0 | 0 | 0 | 100 |
| OCBBF2007762 | 0 | 0 | 0 | 0 | 0 | 0 | 39.066 |
| OCBBF2007829 | 0 | 1.996 | 0 | 1.323 | 0 | 4.047 | 2.728 |
| OCBBF2007892 | 0 | 0 | 0 | 0 | 0 | 0 | 5.292 |
| OCBBF2007931 | 0 | 0 | 0 | 0 | 0 | 0 | 100 |
| OCBBF2007946 | 0 | 0 | 0 | 0 | 0 | 0 | 100 |
| OCBBF2008005 | 0 | 0 | 0 | 0 | 0 | 0 | 21.697 |
| OCBBF2008041 | 0 | 0 | 0 | 5.038 | 0 | 0 | 2.597 |
| OCBBF2008116 | 0 | 0 | 0 | 0 | 0 | 0 | 100 |
| OCBBF2008138 | 0 | 0 | 0 | 0 | 0 | 0 | 100 |
| OCBBF2008144 | 0 | 0 | 0 | 0 | 0 | 0 | 7.165 |
| OCBBF2008283 | 0 | 0 | 0 | 0 | 0 | 0 | 100 |
| OCBBF2008330 | 0 | 0 | 0 | 0 | 0 | 0 | 100 |
| OCBBF2008340 | 0 | 0 | 0 | 0 | 0 | 0 | 47.948 |
| OCBBF2008366 | 0 | 0 | 0 | 0 | 0 | 0 | 15.035 |
| OCBBF2008466 | 0 | 0 | 0 | 0 | 0 | 0 | 10.627 |
| OCBBF2008483 | 0 | 0 | 0 | 0 | 0 | 0 | 10.814 |
| OCBBF2008511 | 0 | 0 | 0 | 0 | 0 | 0 | 41.146 |
| OCBBF2008520 | 0 | 0 | 0 | 0 | 0 | 0 | 100 |
| OCBBF2008569 | 0 | 0 | 0 | 0 | 0 | 0 | 26.039 |
| OCBBF2008586 | 0 | 0 | 0 | 0 | 0 | 0 | 100 |
| OCBBF2008640 | 0 | 0 | 0 | 0 | 0 | 0 | 100 |
| OCBBF2008691 | 0 | 0 | 0 | 0 | 0 | 0 | 100 |
| OCBBF2008701 | 0 | 0 | 0 | 0 | 0 | 0 | 40.389 |
| OCBBF2008724 | 0 | 0 | 0 | 0 | 0 | 0 | 100 |
| OCBBF2008760 | 0 | 0 | 0 | 0 | 0 | 0 | 41.319 |
| OCBBF2008768 | 0 | 0 | 0 | 0 | 0 | 0 | 100 |
| OCBBF2008770 | 0 | 0 | 0 | 0 | 0 | 0 | 33.223 |
| OCBBF2008775 | 0 | 0 | 0 | 0 | 0 | 0 | 55.216 |
| OCBBF2008790 | 0 | 0 | 0 | 0 | 0 | 0 | 100 |
| OCBBF2008814 | 0 | 0 | 0 | 0 | 0 | 0 | 100 |
| OCBBF2008822 | 0 | 0 | 0 | 0 | 0 | 0 | 2.961 |
| OCBBF2009095 | 0 | 0 | 0 | 0 | 0 | 0 | 8.165 |
| OCBBF2009115 | 0 | 0 | 0 | 0 | 0 | 0 | 100 |
| OCBBF2009242 | 0 | 0 | 0 | 0 | 0 | 0 | 3.517 |
| OCBBF2009301 | 0 | 15.068 | 0 | 0 | 0 | 0 | 15.451 |
| OCBBF2009352 | 0 | 0 | 0 | 0 | 0 | 0 | 6.993 |
| OCBBF2009391 | 0 | 0 | 0 | 0 | 0 | 0 | 40.207 |
| OCBBF2009424 | 0 | 0 | 0 | 0 | 0 | 0 | 9.015 |
| OCBBF2009536 | 0 | 0 | 0 | 0 | 0 | 0 | 52.835 |
| OCBBF2009571 | 0 | 0 | 0 | 0 | 0 | 0 | 100 |
| OCBBF2009583 | 0 | 0 | 0 | 0 | 0 | 0 | 100 |
| OCBBF2009603 | 0 | 0 | 0 | 0 | 0 | 0 | 41.108 |
| OCBBF2009772 | 0 | 4.280 | 0 | 5.676 | 0 | 0 | 5.851 |
| OCBBF2009788 | 0 | 0 | 0 | 0 | 0 | 0 | 100 |
| OCBBF2009836 | 0 | 0 | 0 | 0 | 0 | 0 | 10.181 |
| OCBBF2009920 | 0 | 0 | 0 | 0 | 0 | 0 | 54.907 |
| OCBBF2009926 | 0 | 0 | 0 | 0 | 0 | 0 | 100 |
| OCBBF2010040 | 0 | 0 | 0 | 0 | 0 | 0 | 100 |
| OCBBF2010140 | 0 | 0 | 0 | 0 | 0 | 0 | 40.385 |
| OCBBF2010281 | 0 | 0 | 0 | 0 | 0 | 0 | 52.585 |
| OCBBF2010313 | 0 | 0 | 0 | 0 | 0 | 0 | 34.079 |
| OCBBF2010404 | 0 | 0 | 0 | 0 | 0 | 0 | 100 |
| OCBBF2010416 | 0 | 0 | 0 | 0 | 0 | 0 | 100 |
| OCBBF2010420 | 0 | 0 | 0 | 0 | 0 | 0 | 100 |
| OCBBF2010557 | 0 | 0 | 0 | 0 | 0 | 0 | 22.763 |
| OCBBF2010604 | 0 | 0 | 0 | 0 | 0 | 0 | 100 |
| OCBBF2010709 | 0 | 0 | 0 | 0 | 0 | 0 | 100 |
| OCBBF2010792 | 0 | 0 | 0 | 0 | 0 | 0 | 100 |
| OCBBF2010819 | 0 | 0 | 0 | 30.231 | 0 | 0 | 15.583 |
| OCBBF2010830 | 0 | 4.797 | 0 | 2.121 | 0 | 0 | 2.186 |
| OCBBF2010841 | 0 | 0 | 0 | 14.981 | 0 | 0 | 7.722 |
| OCBBF2010843 | 53.980 | 0 | 0 | 0 | 0 | 0 | 18.503 |
| OCBBF2010858 | 0 | 0 | 0 | 16.054 | 0 | 0 | 33.100 |
| OCBBF2010863 | 0 | 0 | 0 | 0 | 0 | 0 | 55.216 |
| OCBBF2010871 | 0 | 0 | 0 | 0 | 0 | 0 | 34.069 |
| OCBBF2010931 | 0 | 0 | 0 | 0 | 0 | 0 | 100 |
| OCBBF2010945 | 0 | 0 | 0 | 0 | 0 | 0 | 51.019 |
| OCBBF2010978 | 0 | 0 | 0 | 0 | 0 | 0 | 100 |
| OCBBF2010989 | 0 | 0 | 0 | 0 | 0 | 0 | 100 |
| OCBBF2011021 | 0 | 0 | 0 | 0 | 0 | 0 | 10.774 |
| OCBBF2011073 | 0 | 6.201 | 0 | 0 | 0 | 0 | 21.195 |
| OCBBF2011137 | 0 | 0 | 0 | 0 | 0 | 0 | 100 |
| OCBBF2011160 | 0 | 0 | 0 | 0 | 0 | 0 | 21.686 |
| OCBBF2011177 | 0 | 0 | 0 | 0 | 0 | 0 | 100 |
| OCBBF2011228 | 0 | 0 | 0 | 0 | 0 | 0 | 53.120 |
| OCBBF2011232 | 0 | 0 | 0 | 0 | 0 | 0 | 39.814 |
| OCBBF2011283 | 0 | 0 | 0 | 0 | 0 | 0 | 80.940 |
| OCBBF2011311 | 0 | 0 | 0 | 0 | 0 | 0 | 100 |
| OCBBF2011536 | 0 | 0 | 0 | 0 | 0 | 0 | 4.957 |
| OCBBF2011625 | 0 | 0 | 0 | 0 | 0 | 0 | 33.259 |
| OCBBF2011669 | 0 | 0 | 0 | 0 | 0 | 0 | 63.640 |
| OCBBF2011685 | 0 | 0 | 0 | 0 | 0 | 0 | 100 |
| OCBBF2011706 | 0 | 0 | 0 | 0 | 0 | 0 | 100 |
| OCBBF2011722 | 0 | 0 | 0 | 0 | 0 | 0 | 100 |
| OCBBF2011759 | 0 | 1.698 | 0 | 2.252 | 0 | 0 | 9.288 |
| OCBBF2011767 | 0 | 0 | 0 | 0 | 0 | 0 | 36.946 |
| OCBBF2011855 | 0 | 0 | 0 | 0 | 0 | 0 | 100 |
| OCBBF2011872 | 0 | 0 | 0 | 0 | 0 | 0 | 100 |
| OCBBF2011887 | 0 | 8.546 | 0 | 0 | 0 | 0 | 17.527 |
| OCBBF2011897 | 0 | 0 | 0 | 0 | 0 | 0 | 21.447 |
| OCBBF2011914 | 0 | 0 | 0 | 0 | 0 | 0 | 18.119 |
| OCBBF2011960 | 0 | 0 | 0 | 0 | 0 | 0 | 100 |
| OCBBF2011981 | 0 | 0 | 0 | 0 | 0 | 0 | 100 |
| OCBBF2012001 | 0 | 0 | 0 | 0 | 0 | 0 | 30.933 |
| OCBBF2012028 | 0 | 0 | 0 | 0 | 0 | 0 | 100 |
| OCBBF2012039 | 0 | 0 | 0 | 0 | 0 | 0 | 9.527 |
| OCBBF2012095 | 0 | 0 | 0 | 0 | 0 | 0 | 19.009 |
| OCBBF2012139 | 0 | 0 | 0 | 0 | 0 | 0 | 100 |
| OCBBF2012191 | 0 | 0 | 0 | 0 | 0 | 0 | 40.207 |
| OCBBF2012262 | 0 | 0 | 0 | 34.956 | 0 | 0 | 36.036 |
| OCBBF2012320 | 0 | 0 | 0 | 0 | 0 | 0 | 100 |
| OCBBF2012436 | 0 | 0 | 0 | 0 | 0 | 0 | 59.836 |
| OCBBF2012525 | 0 | 0 | 0 | 0 | 0 | 0 | 84.002 |
| OCBBF2012553 | 0 | 2.131 | 0 | 0 | 0 | 0 | 4.371 |
| OCBBF2012678 | 0 | 12.535 | 0 | 0 | 0 | 0 | 25.707 |
| OCBBF2012704 | 0 | 37.942 | 0 | 0 | 0 | 0 | 25.938 |
| OCBBF2012714 | 0 | 0 | 0 | 0 | 0 | 0 | 7.211 |
| OCBBF2012755 | 0 | 0 | 0 | 0 | 0 | 0 | 100 |
| OCBBF2012812 | 0 | 0 | 0 | 0 | 0 | 0 | 25.584 |
| OCBBF2012821 | 0 | 0 | 0 | 0 | 0 | 0 | 3.108 |
| OCBBF2012881 | 0 | 0 | 0 | 0 | 0 | 0 | 55.216 |
| OCBBF2012908 | 11.876 | 0 | 0 | 0 | 0 | 0 | 12.212 |
| OCBBF2012936 | 0 | 0 | 0 | 0 | 0 | 0 | 55.403 |
| OCBBF2012990 | 0 | 0 | 0 | 0 | 0 | 0 | 100 |
| OCBBF2013011 | 0 | 0 | 0 | 18.197 | 0 | 0 | 9.380 |
| OCBBF2013049 | 0 | 0 | 0 | 0 | 0 | 0 | 100 |
| OCBBF2013091 | 0 | 0 | 0 | 0 | 0 | 0 | 100 |
| OCBBF2013123 | 0 | 0 | 0 | 0 | 0 | 0 | 28.345 |
| OCBBF2013127 | 0 | 0 | 0 | 0 | 0 | 0 | 100 |
| OCBBF2013149 | 0 | 0 | 0 | 0 | 0 | 0 | 1.927 |
| OCBBF2013208 | 0 | 0 | 0 | 0 | 0 | 0 | 100 |
| OCBBF2013285 | 0 | 0 | 0 | 0 | 0 | 0 | 63.640 |
| OCBBF2013319 | 0 | 0 | 0 | 0 | 0 | 0 | 100 |
| OCBBF2013324 | 0 | 0 | 0 | 0 | 0 | 0 | 46.671 |
| OCBBF2013366 | 9.783 | 4.905 | 0 | 0 | 0 | 0 | 3.353 |
| OCBBF2013481 | 0 | 0 | 0 | 0 | 0 | 0 | 6.792 |
| OCBBF2013507 | 0 | 0 | 0 | 0 | 0 | 0 | 100 |
| OCBBF2013721 | 0 | 0 | 0 | 0 | 0 | 0 | 100 |
| OCBBF2013789 | 0 | 0 | 0 | 0 | 0 | 0 | 37.496 |
| OCBBF2013843 | 0 | 0 | 0 | 0 | 0 | 3.938 | 3.983 |
| OCBBF2013883 | 74.472 | 0 | 0 | 0 | 0 | 0 | 25.528 |
| OCBBF2013926 | 0 | 0 | 0 | 0 | 0 | 0 | 77.076 |
| OCBBF2014052 | 0 | 0 | 0 | 0 | 0 | 0 | 52.585 |
| OCBBF2014064 | 0 | 0 | 0 | 0 | 0 | 0 | 3.394 |
| OCBBF2014089 | 0 | 35.863 | 0 | 0 | 0 | 0 | 24.517 |
| OCBBF2014292 | 3.754 | 0 | 0 | 2.496 | 0 | 7.633 | 25.733 |
| OCBBF2014322 | 0 | 0 | 0 | 0 | 0 | 0 | 25.645 |
| OCBBF2014386 | 0 | 0 | 0 | 43.822 | 0 | 0 | 22.588 |
| OCBBF2014541 | 0 | 0 | 0 | 0 | 0 | 0 | 34.096 |
| OCBBF2014576 | 0 | 0 | 0 | 0 | 0 | 0 | 4.539 |
| OCBBF2014707 | 0 | 0 | 0 | 0 | 0 | 0 | 100 |
| OCBBF2014745 | 0 | 0 | 0 | 0 | 0 | 0 | 63.640 |
| OCBBF2014828 | 0 | 0 | 0 | 0 | 0 | 0 | 100 |
| OCBBF2014873 | 0 | 0 | 0 | 0 | 0 | 0 | 17.619 |
| OCBBF2014880 | 0 | 0 | 0 | 0 | 0 | 0 | 40.207 |
| OCBBF2014928 | 0 | 0 | 0 | 0 | 0 | 0 | 40.207 |
| OCBBF2015031 | 0 | 0 | 0 | 0 | 0 | 0 | 100 |
| OCBBF2015081 | 0 | 0 | 0 | 0 | 0 | 0 | 100 |
| OCBBF2015115 | 0 | 0 | 0 | 43.004 | 0 | 0 | 44.332 |
| OCBBF2015232 | 0 | 0 | 0 | 0 | 0 | 0 | 100 |
| OCBBF2015233 | 0 | 0 | 0 | 0 | 0 | 0 | 77.781 |
| OCBBF2015285 | 0 | 0 | 0 | 0 | 0 | 0 | 100 |
| OCBBF2015334 | 0 | 0 | 0 | 0 | 0 | 0 | 65.522 |
| OCBBF2015335 | 0 | 0 | 0 | 0 | 0 | 0 | 100 |
| OCBBF2015503 | 0 | 0 | 0 | 18.086 | 0 | 0 | 18.645 |
| OCBBF2015506 | 0 | 0 | 0 | 0 | 0 | 0 | 100 |
| OCBBF2015599 | 0 | 0 | 0 | 0 | 0 | 0 | 40.207 |
| OCBBF2015645 | 0 | 0 | 0 | 0 | 0 | 0 | 100 |
| OCBBF2015659 | 0 | 0 | 0 | 0 | 0 | 0 | 100 |
| OCBBF2015751 | 0 | 0 | 0 | 0 | 0 | 0 | 10.561 |
| OCBBF2015797 | 0 | 0 | 0 | 0 | 0 | 0 | 65.522 |
| OCBBF2015931 | 0 | 0 | 0 | 0 | 0 | 0 | 25.025 |
| OCBBF2015980 | 0 | 0 | 0 | 0 | 0 | 0 | 100 |
| OCBBF2016038 | 0 | 0 | 0 | 0 | 0 | 0 | 100 |
| OCBBF2016405 | 0 | 0 | 0 | 0 | 0 | 0 | 28.747 |
| OCBBF2016467 | 0 | 0 | 0 | 0 | 0 | 0 | 100 |
| OCBBF2016590 | 0 | 0 | 0 | 0 | 0 | 0 | 100 |
| OCBBF2016591 | 0 | 0 | 0 | 0 | 0 | 0 | 100 |
| OCBBF2016612 | 0 | 0 | 0 | 0 | 0 | 0 | 52.585 |
| OCBBF2016689 | 0 | 0 | 0 | 0 | 0 | 0 | 77.781 |
| OCBBF2016690 | 0 | 0 | 0 | 0 | 0 | 0 | 100 |
| OCBBF2016729 | 0 | 0 | 0 | 0 | 0 | 0 | 11.324 |
| OCBBF2016841 | 22.139 | 0 | 0 | 0 | 0 | 0 | 7.589 |
| OCBBF2016928 | 0 | 0 | 0 | 0 | 0 | 0 | 13.608 |
| OCBBF2017035 | 0 | 0 | 0 | 2.922 | 0 | 8.935 | 6.024 |
| OCBBF2017055 | 0 | 0 | 0 | 0 | 0 | 0 | 22.290 |
| OCBBF2017069 | 0 | 0 | 0 | 0 | 0 | 0 | 100 |
| OCBBF2017306 | 0 | 0 | 0 | 0 | 0 | 0 | 55.403 |
| OCBBF2017325 | 0 | 0 | 0 | 0 | 0 | 0 | 100 |
| OCBBF2017398 | 0 | 2.338 | 0 | 3.100 | 0 | 0 | 9.588 |
| OCBBF2017458 | 0 | 10.117 | 0 | 0 | 0 | 0 | 20.748 |
| OCBBF2017489 | 0 | 0 | 0 | 0 | 0 | 0 | 100 |
| OCBBF2017516 | 0 | 0 | 0 | 0 | 0 | 0 | 100 |
| OCBBF2017665 | 0 | 0 | 0 | 0 | 0 | 0 | 100 |
| OCBBF2017754 | 0 | 0 | 0 | 9.049 | 0 | 0 | 27.986 |
| OCBBF2017791 | 0 | 4.655 | 0 | 6.174 | 0 | 0 | 1.591 |
| OCBBF2017815 | 0 | 0 | 0 | 0 | 0 | 0 | 65.522 |
| OCBBF2017882 | 0 | 0 | 0 | 0 | 0 | 0 | 8.864 |
| OCBBF2017888 | 0 | 0 | 0 | 0 | 0 | 0 | 21.165 |
| OCBBF2017893 | 0 | 0 | 0 | 0 | 0 | 0 | 4.310 |
| OCBBF2017920 | 0 | 0 | 0 | 31.033 | 0 | 0 | 31.992 |
| OCBBF2018012 | 0 | 0 | 0 | 0 | 0 | 0 | 3.487 |
| OCBBF2018014 | 0 | 0 | 0 | 0 | 0 | 0 | 100 |
| OCBBF2018084 | 0 | 0 | 0 | 0 | 0 | 0 | 16.434 |
| OCBBF2018167 | 0 | 0 | 0 | 0 | 0 | 0 | 100 |
| OCBBF2018206 | 0 | 0 | 0 | 0 | 0 | 0 | 54.907 |
| OCBBF2018229 | 0 | 0 | 0 | 0 | 0 | 0 | 100 |
| OCBBF2018234 | 0 | 11.770 | 0 | 0 | 0 | 0 | 16.093 |
| OCBBF2018362 | 0 | 0 | 0 | 0 | 0 | 0 | 57.154 |
| OCBBF2018380 | 0 | 0 | 0 | 0 | 0 | 0 | 100 |
| OCBBF2018563 | 0 | 0 | 0 | 0 | 0 | 0 | 25.548 |
| OCBBF2018581 | 0 | 7.818 | 0 | 0 | 0 | 0 | 21.378 |
| OCBBF2018618 | 0 | 0 | 0 | 0 | 0 | 0 | 40.294 |
| OCBBF2018663 | 0 | 0 | 0 | 0 | 0 | 0 | 100 |
| OCBBF2018687 | 0 | 0 | 0 | 0 | 0 | 0 | 100 |
| OCBBF2018707 | 0 | 0 | 0 | 0 | 0 | 0 | 2.794 |
| OCBBF2018827 | 0 | 0 | 0 | 11.826 | 0 | 0 | 6.095 |
| OCBBF2018828 | 0 | 0 | 0 | 0 | 0 | 0 | 100 |
| OCBBF2018873 | 0 | 0 | 39.876 | 0 | 0 | 10.213 | 5.164 |
| OCBBF2018934 | 0 | 0 | 0 | 0 | 0 | 0 | 100 |
| OCBBF2018956 | 0 | 0 | 0 | 4.079 | 0 | 0 | 2.102 |
| OCBBF2019108 | 0 | 0 | 0 | 0 | 0 | 0 | 42.690 |
| OCBBF2019195 | 0 | 0 | 0 | 0 | 0 | 0 | 100 |
| OCBBF2019327 | 0 | 0 | 0 | 0 | 0 | 0 | 100 |
| OCBBF2019684 | 0 | 0 | 0 | 0 | 0 | 0 | 100 |
| OCBBF2019761 | 0 | 0 | 0 | 0 | 0 | 0 | 100 |
| OCBBF2019823 | 0 | 0 | 0 | 0 | 0 | 0 | 100 |
| OCBBF2019950 | 0 | 0 | 0 | 0 | 0 | 0 | 54.907 |
| OCBBF2020048 | 0 | 0 | 0 | 0 | 0 | 0 | 65.522 |
| OCBBF2020318 | 0 | 0 | 0 | 0 | 0 | 0 | 59.836 |
| OCBBF2020343 | 0 | 0 | 0 | 0 | 0 | 0 | 63.640 |
| OCBBF2020453 | 0 | 0 | 0 | 0 | 0 | 0 | 100 |
| OCBBF2020741 | 0 | 0 | 0 | 0 | 0 | 0 | 17.527 |
| OCBBF2020801 | 0 | 0 | 0 | 0 | 0 | 0 | 59.836 |
| OCBBF2020825 | 0 | 0 | 0 | 0 | 0 | 0 | 100 |
| OCBBF2020838 | 0 | 0 | 0 | 0 | 0 | 0 | 100 |
| OCBBF2021020 | 0 | 0 | 0 | 0 | 0 | 0 | 3.312 |
| OCBBF2021214 | 0 | 0 | 0 | 0 | 0 | 0 | 100 |
| OCBBF2021286 | 0 | 0 | 0 | 0 | 0 | 0 | 22.928 |
| OCBBF2021323 | 0 | 0 | 0 | 0 | 0 | 0 | 100 |
| OCBBF2021518 | 0 | 0 | 0 | 0 | 0 | 0 | 100 |
| OCBBF2021788 | 0 | 0 | 0 | 0 | 0 | 0 | 100 |
| OCBBF2021833 | 0 | 0 | 0 | 0 | 0 | 0 | 36.034 |
| OCBBF2022351 | 0 | 0 | 0 | 0 | 0 | 0 | 63.640 |
| OCBBF2022573 | 0 | 0 | 0 | 0 | 0 | 0 | 100 |
| OCBBF2022574 | 0 | 0 | 0 | 0 | 0 | 0 | 100 |
| OCBBF2023162 | 0 | 0 | 0 | 0 | 0 | 0 | 12.773 |
| OCBBF2023598 | 0 | 0 | 0 | 0 | 0 | 0 | 100 |
| OCBBF2023643 | 0 | 0 | 0 | 0 | 0 | 0 | 100 |
| OCBBF2024463 | 0 | 0 | 0 | 0 | 0 | 0 | 100 |
| OCBBF2024589 | 0 | 0 | 0 | 0 | 0 | 0 | 100 |
| OCBBF2024719 | 0 | 0 | 0 | 0 | 0 | 0 | 36.847 |
| OCBBF2024779 | 0 | 0 | 0 | 0 | 0 | 0 | 100 |
| OCBBF2024781 | 0 | 0 | 0 | 0 | 0 | 0 | 100 |
| OCBBF2024850 | 0 | 0 | 0 | 0 | 0 | 0 | 5.522 |
| OCBBF2025028 | 0 | 0 | 0 | 0 | 0 | 0 | 100 |
| OCBBF2025115 | 0 | 0 | 0 | 0 | 0 | 0 | 100 |
| OCBBF2025198 | 0 | 0 | 0 | 0 | 0 | 0 | 100 |
| OCBBF2025451 | 0 | 0 | 0 | 0 | 0 | 0 | 100 |
| OCBBF2025458 | 0 | 0 | 0 | 0 | 0 | 0 | 100 |
| OCBBF2025527 | 0 | 0 | 0 | 0 | 0 | 0 | 63.640 |
| OCBBF2025631 | 0 | 0 | 0 | 0 | 0 | 0 | 100 |
| OCBBF2025730 | 0 | 0 | 0 | 0 | 0 | 0 | 100 |
| OCBBF2026025 | 0 | 0 | 0 | 0 | 0 | 0 | 100 |
| OCBBF2026035 | 0 | 0 | 0 | 0 | 0 | 0 | 100 |
| OCBBF2026144 | 0 | 0 | 0 | 0 | 0 | 0 | 100 |
| OCBBF2026368 | 0 | 0 | 0 | 0 | 0 | 0 | 30.795 |
| OCBBF2026645 | 0 | 0 | 0 | 0 | 0 | 0 | 100 |
| OCBBF2026649 | 0 | 0 | 0 | 0 | 0 | 0 | 63.640 |
| OCBBF2026690 | 0 | 0 | 0 | 0 | 0 | 0 | 100 |
| OCBBF2026981 | 0 | 0 | 0 | 0 | 0 | 0 | 100 |
| OCBBF2027148 | 0 | 0 | 0 | 0 | 0 | 0 | 28.907 |
| OCBBF2027197 | 0 | 0 | 0 | 0 | 0 | 0 | 63.640 |
| OCBBF2027354 | 0 | 0 | 0 | 0 | 0 | 0 | 100 |
| OCBBF2027423 | 0 | 0 | 0 | 0 | 0 | 0 | 100 |
| OCBBF2027478 | 0 | 0 | 0 | 0 | 0 | 0 | 100 |
| OCBBF2027481 | 0 | 0 | 0 | 0 | 0 | 0 | 100 |
| OCBBF2027511 | 0 | 0 | 0 | 0 | 0 | 0 | 100 |
| OCBBF2027661 | 0 | 0 | 0 | 0 | 0 | 0 | 46.671 |
| OCBBF2027728 | 0 | 0 | 0 | 0 | 0 | 0 | 100 |
| OCBBF2027782 | 0 | 0 | 0 | 0 | 0 | 0 | 100 |
| OCBBF2027868 | 0 | 0 | 0 | 0 | 0 | 0 | 100 |
| OCBBF2028055 | 0 | 0 | 0 | 0 | 0 | 0 | 55.403 |
| OCBBF2028173 | 0 | 0 | 0 | 0 | 0 | 0 | 100 |
| OCBBF2028421 | 0 | 0 | 0 | 0 | 0 | 0 | 52.585 |
| OCBBF2029471 | 0 | 0 | 0 | 0 | 0 | 0 | 100 |
| OCBBF2029901 | 0 | 0 | 0 | 0 | 0 | 0 | 100 |
| OCBBF2030116 | 0 | 0 | 0 | 0 | 0 | 0 | 54.907 |
| OCBBF2030329 | 0 | 0 | 0 | 0 | 0 | 0 | 55.216 |
| OCBBF2030354 | 0 | 0 | 0 | 0 | 0 | 0 | 100 |
| OCBBF2030517 | 0 | 0 | 0 | 0 | 0 | 0 | 100 |
| OCBBF2030574 | 0 | 0 | 0 | 0 | 0 | 0 | 100 |
| OCBBF2030708 | 0 | 0 | 0 | 0 | 0 | 0 | 100 |
| OCBBF2030927 | 0 | 0 | 0 | 0 | 0 | 0 | 55.216 |
| OCBBF2030963 | 0 | 0 | 0 | 0 | 0 | 0 | 100 |
| OCBBF2031167 | 0 | 0 | 0 | 0 | 0 | 0 | 100 |
| OCBBF2031198 | 0 | 0 | 0 | 0 | 0 | 0 | 100 |
| OCBBF2031366 | 0 | 0 | 0 | 0 | 0 | 0 | 100 |
| OCBBF2031561 | 0 | 0 | 0 | 0 | 0 | 0 | 39.003 |
| OCBBF2032152 | 0 | 0 | 0 | 0 | 0 | 0 | 100 |
| OCBBF2032274 | 0 | 0 | 0 | 0 | 0 | 0 | 100 |
| OCBBF2032539 | 0 | 0 | 0 | 0 | 0 | 0 | 100 |
| OCBBF2032590 | 0 | 0 | 0 | 0 | 0 | 0 | 100 |
| OCBBF2032599 | 0 | 0 | 0 | 0 | 0 | 0 | 100 |
| OCBBF2032611 | 0 | 0 | 0 | 0 | 0 | 0 | 100 |
| OCBBF2032671 | 0 | 0 | 0 | 0 | 0 | 0 | 100 |
| OCBBF2033295 | 0 | 0 | 0 | 12.672 | 0 | 0 | 39.191 |
| OCBBF2033413 | 0 | 0 | 0 | 0 | 0 | 0 | 100 |
| OCBBF2033869 | 0 | 0 | 0 | 0 | 0 | 0 | 100 |
| OCBBF2033948 | 0 | 0 | 0 | 0 | 0 | 0 | 100 |
| OCBBF2034333 | 0 | 0 | 0 | 0 | 0 | 0 | 100 |
| OCBBF2034529 | 0 | 0 | 0 | 0 | 0 | 0 | 100 |
| OCBBF2034637 | 0 | 0 | 0 | 0 | 0 | 0 | 100 |
| OCBBF2034906 | 0 | 0 | 0 | 0 | 0 | 0 | 100 |
| OCBBF2035110 | 0 | 0 | 0 | 0 | 0 | 0 | 100 |
| OCBBF2035214 | 0 | 0 | 0 | 0 | 0 | 0 | 55.216 |
| OCBBF2035226 | 0 | 0 | 0 | 1.416 | 0 | 0 | 0.730 |
| OCBBF2035390 | 0 | 0 | 0 | 0 | 0 | 0 | 100 |
| OCBBF2035564 | 0 | 0 | 0 | 0 | 0 | 0 | 100 |
| OCBBF2035658 | 0 | 0 | 0 | 0 | 0 | 0 | 100 |
| OCBBF2035823 | 0 | 0 | 0 | 0 | 0 | 0 | 42.690 |
| OCBBF2035885 | 0 | 0 | 0 | 0 | 0 | 0 | 100 |
| OCBBF2035916 | 0 | 0 | 0 | 0 | 0 | 0 | 100 |
| OCBBF2036019 | 0 | 0 | 0 | 0 | 0 | 0 | 100 |
| OCBBF2036031 | 0 | 0 | 0 | 0 | 0 | 0 | 100 |
| OCBBF2036225 | 0 | 0 | 0 | 0 | 0 | 0 | 100 |
| OCBBF2036476 | 0 | 0 | 0 | 0 | 0 | 0 | 100 |
| OCBBF2036743 | 0 | 0 | 0 | 0 | 0 | 0 | 27.462 |
| OCBBF2036752 | 0 | 0 | 0 | 0 | 0 | 0 | 100 |
| OCBBF2037068 | 54.224 | 27.189 | 0 | 0 | 0 | 0 | 18.587 |
| OCBBF2037340 | 0 | 0 | 0 | 0 | 0 | 0 | 100 |
| OCBBF2037398 | 0 | 0 | 0 | 0 | 0 | 0 | 100 |
| OCBBF2037464 | 0 | 0 | 0 | 0 | 0 | 0 | 100 |
| OCBBF2037547 | 0 | 0 | 0 | 0 | 0 | 0 | 100 |
| OCBBF2037598 | 0 | 0 | 0 | 0 | 0 | 0 | 100 |
| OCBBF2037638 | 0 | 0 | 0 | 0 | 0 | 0 | 100 |
| OCBBF2038238 | 0 | 0 | 0 | 0 | 0 | 0 | 100 |
| OCBBF2038285 | 0 | 0 | 0 | 0 | 0 | 0 | 100 |
| OCBBF2038317 | 0 | 0 | 0 | 0 | 0 | 0 | 12.245 |
| OCBBF3000097 | 0 | 0 | 0 | 0 | 0 | 0 | 100 |
| OCBBF3000213 | 0 | 0 | 0 | 0 | 0 | 0 | 100 |
| OCBBF3000296 | 0 | 0 | 0 | 0 | 0 | 0 | 47.069 |
| OCBBF3000323 | 0 | 0 | 0 | 0 | 0 | 0 | 4.132 |
| OCBBF3000372 | 0 | 0 | 0 | 0 | 0 | 0 | 100 |
| OCBBF3000380 | 0 | 0 | 0 | 0 | 0 | 0 | 100 |
| OCBBF3000743 | 0 | 0 | 0 | 0 | 0 | 0 | 100 |
| OCBBF3000830 | 0 | 0 | 0 | 0 | 0 | 0 | 100 |
| OCBBF3001076 | 0 | 0 | 0 | 0 | 0 | 0 | 100 |
| OCBBF3001202 | 0 | 0 | 0 | 0 | 0 | 0 | 100 |
| OCBBF3001333 | 0 | 5.839 | 0 | 7.743 | 0 | 0 | 11.974 |
| OCBBF3001616 | 0 | 0 | 0 | 0 | 0 | 0 | 100 |
| OCBBF3002553 | 0 | 0 | 0 | 0 | 0 | 0 | 100 |
| OCBBF3002600 | 0 | 0 | 0 | 0 | 0 | 0 | 100 |
| OCBBF3002654 | 0 | 0 | 0 | 0 | 0 | 0 | 100 |
| OCBBF3003103 | 0 | 0 | 0 | 0 | 0 | 0 | 55.403 |
| OCBBF3003209 | 0 | 0 | 0 | 0 | 0 | 0 | 100 |
| OCBBF3003592 | 0 | 0 | 0 | 0 | 0 | 0 | 100 |
| OCBBF3003745 | 0 | 0 | 0 | 0 | 0 | 0 | 100 |
| OCBBF3003761 | 0 | 0 | 0 | 0 | 0 | 0 | 100 |
| OCBBF3004264 | 0 | 0 | 0 | 0 | 0 | 0 | 100 |
| OCBBF3004314 | 0 | 0 | 0 | 0 | 0 | 0 | 100 |
| OCBBF3004487 | 0 | 0 | 0 | 0 | 0 | 0 | 11.257 |
| OCBBF3004972 | 0 | 0 | 0 | 0 | 0 | 0 | 100 |
| OCBBF3005330 | 0 | 0 | 0 | 0 | 0 | 0 | 100 |
| OCBBF3005597 | 0 | 0 | 0 | 0 | 0 | 0 | 100 |
| OCBBF3005843 | 0 | 0 | 0 | 0 | 0 | 0 | 100 |
| OCBBF3006802 | 0 | 0 | 0 | 0 | 0 | 0 | 100 |
| OCBBF3006986 | 0 | 0 | 0 | 0 | 0 | 0 | 100 |
| OCBBF3007078 | 0 | 0 | 0 | 0 | 0 | 0 | 100 |
| OCBBF3007704 | 0 | 0 | 0 | 0 | 0 | 0 | 100 |
| OCBBF3008230 | 0 | 0 | 0 | 0 | 0 | 0 | 37.069 |
| OCBBF3008392 | 0 | 0 | 0 | 0 | 0 | 0 | 100 |
| OCBBF3008835 | 0 | 0 | 0 | 0 | 0 | 0 | 52.835 |
| OCBBF3009192 | 0 | 0 | 0 | 0 | 0 | 0 | 100 |
| OCBBF3009244 | 0 | 0 | 0 | 0 | 0 | 0 | 42.785 |
| OCBBF3009279 | 0 | 0 | 0 | 0 | 0 | 0 | 100 |
| PEBLM1000066 | 0 | 0 | 0 | 0 | 0 | 0 | 5.887 |
| PEBLM1000144 | 0 | 10.899 | 0 | 0 | 0 | 0 | 0 |
| PEBLM2000147 | 0 | 0 | 0 | 0 | 0 | 0 | 4.754 |
| PEBLM2000222 | 0 | 2.111 | 0 | 0 | 0 | 0 | 0 |
| PEBLM2000308 | 0 | 0 | 0 | 4.778 | 0 | 0 | 9.851 |
| PEBLM2000326 | 0 | 0 | 0 | 0 | 0 | 0 | 13.487 |
| PEBLM2000395 | 0 | 3.308 | 0 | 4.387 | 0 | 0 | 3.392 |
| PEBLM2001072 | 0 | 0 | 0 | 4.371 | 0 | 0 | 2.253 |
| PEBLM2004452 | 0 | 2.453 | 0 | 3.253 | 0 | 0 | 5.030 |
| PEBLM2006366 | 0 | 5.030 | 0 | 0 | 0 | 0 | 3.438 |
| PEBLM2006709 | 0 | 10.110 | 0 | 0 | 0 | 0 | 0 |
| PEBLM2007188 | 0 | 0 | 0 | 0 | 0 | 0 | 10.845 |
| PERIC2002766 | 0 | 0 | 0 | 0 | 0 | 0 | 15.571 |
| PERIC2005117 | 0 | 0 | 0 | 0 | 0 | 0 | 5.337 |
| PERIC2007068 | 0 | 2.804 | 0 | 3.719 | 0 | 0 | 3.834 |
| PLACE5000013 | 0 | 2.885 | 0 | 7.651 | 0 | 0 | 0 |
| PLACE5000113 | 0 | 12.506 | 0 | 16.586 | 0 | 0 | 4.275 |
| PLACE5000159 | 0 | 4.130 | 0 | 16.432 | 0 | 0 | 14.117 |
| PLACE6000263 | 0 | 5.526 | 0 | 0 | 0 | 0 | 0 |
| PLACE6000379 | 3.201 | 0.963 | 0 | 1.277 | 0 | 0 | 1.755 |
| PLACE6001064 | 0 | 13.553 | 0 | 9.679 | 0 | 0 | 1.425 |
| PLACE6001281 | 0 | 6.256 | 0 | 0 | 0 | 0 | 4.277 |
| PLACE6001443 | 0 | 3.569 | 0 | 0 | 0 | 0 | 0 |
| PLACE6001923 | 22.779 | 0 | 0 | 0 | 0 | 0 | 0 |
| PLACE6002084 | 6.692 | 0 | 0 | 0.890 | 0 | 0 | 1.376 |
| PLACE6002102 | 0 | 7.700 | 0 | 3.404 | 0 | 0 | 22.809 |
| PLACE6002960 | 0 | 0 | 0 | 0 | 0 | 8.180 | 4.136 |
| PLACE6003383 | 0 | 0 | 0 | 0 | 0 | 8.796 | 0 |
| PLACE6003393 | 0 | 0 | 0 | 0 | 0 | 55.964 | 0 |
| PLACE6003745 | 0 | 9.549 | 0 | 0 | 0 | 0 | 0 |
| PLACE6004005 | 14.313 | 5.383 | 0 | 0 | 0 | 0 | 1.227 |
| PLACE6004312 | 0 | 0 | 0 | 0 | 0 | 0 | 1.498 |
| PLACE6004397 | 0 | 3.340 | 0 | 8.860 | 0 | 0 | 0 |
| PLACE6004931 | 0 | 11.899 | 0 | 0 | 0 | 0 | 0 |
| PLACE6005482 | 0 | 9.610 | 0 | 0 | 0 | 0 | 0 |
| PLACE6006042 | 0 | 0 | 0 | 0 | 0 | 0 | 6.763 |
| PLACE6006137 | 0 | 0 | 0 | 8.232 | 0 | 0 | 4.243 |
| PLACE6006988 | 0 | 0 | 0 | 0 | 0 | 0 | 41.108 |
| PLACE6008793 | 5.746 | 0 | 0 | 3.821 | 0 | 0 | 0 |
| PLACE6010484 | 5.602 | 0 | 0 | 0 | 0 | 0 | 0 |
| PLACE6011041 | 0 | 0 | 0 | 0 | 0 | 0 | 3.380 |
| PLACE6011057 | 0 | 0 | 0 | 5.432 | 0 | 0 | 2.800 |
| PLACE6011334 | 0 | 0 | 0 | 0 | 0 | 0 | 11.165 |
| PLACE6011627 | 0 | 0 | 0 | 0 | 0 | 0 | 41.108 |
| PLACE6011970 | 0 | 0 | 0 | 0 | 0 | 0 | 32.218 |
| PLACE6016210 | 45.894 | 0 | 0 | 0 | 0 | 0 | 0 |
| PLACE6018938 | 0 | 0 | 0 | 18.491 | 0 | 0 | 0 |
| PLACE6019385 | 0 | 0 | 0 | 0 | 0 | 0 | 5.039 |
| PLACE7004103 | 0 | 9.400 | 0 | 6.234 | 0 | 9.531 | 1.607 |
| PROST1000097 | 0 | 0 | 0 | 0 | 0 | 0 | 0.730 |
| PROST1000144 | 0 | 0 | 0 | 0 | 0 | 0 | 5.843 |
| PROST1000199 | 0 | 1.328 | 0 | 1.761 | 0 | 0 | 0.908 |
| PROST1000215 | 0 | 0 | 0 | 0 | 0 | 0 | 12.708 |
| PROST1000220 | 0 | 0 | 0 | 0 | 0 | 30.022 | 0 |
| PROST1000226 | 0 | 6.503 | 0 | 0 | 0 | 0 | 0 |
| PROST1000322 | 0 | 0 | 0 | 57.613 | 0 | 0 | 0 |
| PROST1000526 | 9.156 | 0 | 0 | 0 | 0 | 0 | 0 |
| PROST1000564 | 0 | 0 | 0 | 3.240 | 0 | 0 | 0 |
| PROST2000079 | 0 | 0 | 0 | 7.214 | 0 | 0 | 0 |
| PROST2000138 | 0 | 0.945 | 0 | 0.209 | 0 | 0.639 | 0.108 |
| PROST2000274 | 0 | 0 | 0 | 24.872 | 0 | 0 | 0 |
| PROST2000284 | 0 | 0 | 0 | 16.880 | 0 | 0 | 4.350 |
| PROST2000325 | 0 | 0 | 0 | 17.061 | 0 | 0 | 0 |
| PROST2000760 | 0 | 0 | 0 | 0 | 0 | 0 | 3.485 |
| PROST2001180 | 0 | 0 | 0 | 5.984 | 0 | 0 | 0 |
| PROST2001213 | 0 | 0 | 0 | 20.091 | 0 | 0 | 0 |
| PROST2001552 | 0 | 3.164 | 0 | 2.098 | 0 | 0 | 0 |
| PROST2002227 | 0 | 0 | 73.145 | 0 | 0 | 0 | 0 |
| PROST2002651 | 0 | 0 | 0 | 0 | 0 | 22.039 | 7.430 |
| PROST2002960 | 0 | 24.426 | 0 | 0 | 0 | 0 | 16.698 |
| PROST2003232 | 0 | 0 | 0 | 0 | 0 | 0 | 0.848 |
| PROST2003338 | 0 | 0 | 0 | 2.698 | 0 | 0 | 0 |
| PROST2003396 | 0 | 0 | 0 | 0 | 0 | 0 | 7.422 |
| PROST2003517 | 0 | 0 | 0 | 0 | 0 | 0 | 3.271 |
| PROST2004095 | 0 | 0 | 0 | 2.256 | 0 | 3.449 | 5.233 |
| PROST2004258 | 0 | 0 | 0 | 9.195 | 0 | 0 | 0 |
| PROST2004270 | 0 | 1.069 | 16.921 | 4.252 | 0 | 0 | 7.305 |
| PROST2004416 | 2.464 | 2.471 | 19.566 | 0 | 0 | 10.022 | 0 |
| PROST2004570 | 27.165 | 0 | 0 | 0 | 0 | 0 | 0 |
| PROST2004739 | 0 | 0 | 0 | 0 | 0 | 0 | 27.713 |
| PROST2005143 | 0 | 0 | 0 | 0 | 0 | 0 | 8.427 |
| PROST2005272 | 0 | 0 | 0 | 0 | 0 | 0 | 10.202 |
| PROST2005793 | 0 | 0 | 0 | 0 | 0 | 0 | 4.872 |
| PROST2006030 | 0 | 13.752 | 0 | 9.119 | 0 | 0 | 0 |
| PROST2006260 | 0 | 0 | 0 | 0 | 0 | 0 | 10.117 |
| PROST2006343 | 0 | 0 | 0 | 0 | 0 | 32.585 | 0 |
| PROST2006536 | 0 | 4.033 | 0 | 10.696 | 0 | 0 | 0 |
| PROST2007200 | 0 | 0 | 0 | 0 | 0 | 0 | 44.022 |
| PROST2007871 | 39.973 | 0 | 0 | 0 | 0 | 0 | 13.702 |
| PROST2008271 | 0 | 0 | 0 | 0 | 0 | 0 | 9.737 |
| PROST2008489 | 2.347 | 1.177 | 0 | 0 | 0 | 0 | 0 |
| PROST2008770 | 14.135 | 0 | 0 | 1.880 | 0 | 0 | 2.907 |
| PROST2009400 | 0 | 21.671 | 0 | 0 | 0 | 0 | 14.815 |
| PROST2010782 | 2.644 | 0 | 0 | 0.586 | 0 | 0 | 0 |
| PROST2011577 | 0 | 0 | 0 | 0 | 0 | 0 | 1.278 |
| PROST2012007 | 0 | 2.062 | 0 | 0 | 0 | 0 | 0 |
| PROST2012448 | 0 | 0 | 0 | 0 | 0 | 0 | 10.136 |
| PROST2012888 | 0 | 0 | 0 | 40.462 | 0 | 0 | 0 |
| PROST2012890 | 0 | 27.718 | 0 | 0 | 0 | 0 | 0 |
| PROST2015137 | 0 | 0 | 0 | 0 | 0 | 0 | 22.826 |
| PROST2015332 | 0 | 0 | 0 | 0 | 0 | 0 | 3.202 |
| PROST2016918 | 0 | 0 | 0 | 0 | 0 | 0 | 7.895 |
| PROST2017203 | 0 | 0 | 0 | 0 | 0 | 9.571 | 1.613 |
| PROST2017692 | 0 | 2.678 | 0 | 0 | 0 | 0 | 0 |
| PROST2017910 | 0 | 0 | 0 | 25.185 | 0 | 0 | 0 |
| PROST2018607 | 0 | 0 | 0 | 40.462 | 0 | 0 | 0 |
| PROST2019253 | 0 | 0 | 0 | 0 | 0 | 0 | 8.349 |
| PUAEN1000057 | 0 | 7.462 | 0 | 4.948 | 0 | 0 | 7.651 |
| PUAEN1000065 | 0 | 0 | 0 | 1.614 | 0 | 9.872 | 0 |
| PUAEN1000161 | 0 | 0 | 0 | 0 | 0 | 0 | 7.166 |
| PUAEN1000164 | 0 | 0.723 | 0 | 0.959 | 0 | 0 | 0 |
| PUAEN1000239 | 11.235 | 0 | 0 | 0 | 0 | 0 | 23.107 |
| PUAEN1000322 | 0 | 4.646 | 0 | 0 | 0 | 0 | 0 |
| PUAEN2000374 | 1.160 | 0.582 | 0 | 2.314 | 0 | 2.359 | 1.590 |
| PUAEN2000497 | 0 | 4.685 | 0 | 0 | 0 | 0 | 0 |
| PUAEN2002489 | 0 | 0 | 0 | 0 | 0 | 0 | 8.106 |
| PUAEN2003408 | 0 | 0 | 0 | 0 | 0 | 0 | 3.741 |
| PUAEN2006335 | 0 | 0 | 0 | 0 | 0 | 0 | 15.755 |
| PUAEN2007044 | 0 | 13.024 | 0 | 3.455 | 0 | 0 | 3.561 |
| PUAEN2007350 | 0 | 24.360 | 0 | 0 | 0 | 0 | 0 |
| PUAEN2008815 | 0 | 0 | 0 | 29.929 | 0 | 0 | 0 |
| RECTM1000051 | 0 | 0 | 0 | 0 | 0 | 0 | 2.867 |
| RECTM2000510 | 10.923 | 0 | 0 | 0 | 0 | 0 | 0 |
| RECTM2001347 | 0 | 0 | 0 | 0 | 0 | 0 | 5.153 |
| SALGL1000047 | 0 | 0.538 | 0 | 0.714 | 0 | 0 | 0.368 |
| SALGL1000157 | 0 | 1.654 | 0 | 0.313 | 0 | 0 | 0 |
| SKMUS1000022 | 0 | 0.756 | 0 | 1.003 | 0 | 0 | 0.517 |
| SKMUS1000064 | 0 | 0.853 | 0 | 0 | 0 | 0 | 0 |
| SKMUS1000084 | 0 | 2.799 | 0 | 0 | 0 | 0 | 7.653 |
| SKMUS1000138 | 0 | 0 | 0 | 0 | 0 | 1.416 | 0 |
| SKMUS1000186 | 1.455 | 0.729 | 0 | 0 | 0 | 0 | 0.499 |
| SKMUS2000198 | 0 | 0 | 0 | 0 | 0 | 23.281 | 0 |
| SKMUS2000361 | 0 | 1.943 | 0 | 0 | 0 | 0 | 0 |
| SKMUS2000467 | 0 | 20.503 | 0 | 0 | 0 | 0 | 0 |
| SKMUS2000679 | 0 | 0 | 0 | 0 | 0 | 4.937 | 0 |
| SKMUS2000931 | 1.972 | 2.967 | 0 | 1.311 | 0 | 4.010 | 2.028 |
| SKMUS2001147 | 0 | 0 | 0 | 6.681 | 0 | 0 | 3.444 |
| SKMUS2001323 | 0 | 0 | 50.550 | 0 | 0 | 0 | 0 |
| SKMUS2001454 | 2.446 | 3.067 | 0 | 1.627 | 0 | 0 | 2.096 |
| SKMUS2001634 | 0 | 0 | 0 | 0 | 0 | 9.649 | 4.879 |
| SKMUS2002084 | 22.628 | 11.346 | 0 | 0 | 0 | 0 | 0 |
| SKMUS2004908 | 0 | 5.969 | 0 | 23.749 | 0 | 0 | 8.161 |
| SKNMC1000110 | 0 | 0 | 0 | 17.451 | 0 | 26.682 | 0 |
| SKNMC1000168 | 3.035 | 0 | 0 | 0 | 0 | 0 | 2.081 |
| SKNMC1000229 | 0 | 0 | 0 | 25.340 | 0 | 0 | 0 |
| SKNMC1000264 | 0 | 0 | 0 | 12.641 | 0 | 0 | 0 |
| SKNMC2000065 | 0 | 7.847 | 0 | 7.805 | 0 | 0 | 9.388 |
| SKNMC2000097 | 0 | 0 | 12.826 | 1.074 | 0 | 0 | 1.107 |
| SKNMC2000635 | 1.950 | 2.608 | 0 | 0.865 | 0 | 0 | 0 |
| SKNMC2000649 | 0 | 1.658 | 0 | 2.199 | 0 | 6.724 | 1.133 |
| SKNMC2000698 | 0 | 0 | 0 | 4.978 | 0 | 0 | 2.566 |
| SKNMC2000877 | 0 | 0 | 0 | 0 | 0 | 0 | 1.472 |
| SKNMC2001324 | 0 | 0 | 0 | 0 | 0 | 0 | 8.022 |
| SKNMC2002402 | 0 | 0 | 0 | 0 | 0 | 0 | 9.158 |
| SKNMC2006173 | 0 | 13.984 | 0 | 0 | 0 | 0 | 9.559 |
| SKNSH1000002 | 0 | 2.612 | 0 | 0 | 0 | 10.593 | 0 |
| SKNSH1000086 | 0 | 0 | 0 | 20.579 | 0 | 0 | 10.607 |
| SKNSH1000101 | 0 | 0 | 0 | 0 | 0 | 0 | 0.798 |
| SKNSH2000051 | 0 | 0 | 0 | 0 | 0 | 0 | 41.985 |
| SKNSH2000101 | 0 | 0 | 0 | 0 | 0 | 0 | 8.186 |
| SKNSH2000250 | 0 | 0 | 0 | 0 | 0 | 0 | 3.500 |
| SKNSH2000347 | 0 | 0 | 0 | 0 | 0 | 0 | 5.575 |
| SKNSH2000819 | 0 | 4.523 | 0 | 0 | 0 | 0 | 0 |
| SKNSH2001931 | 0 | 0 | 0 | 24.633 | 0 | 0 | 6.348 |
| SKNSH2002325 | 0 | 6.937 | 0 | 0 | 0 | 0 | 0 |
| SKNSH2005061 | 0 | 3.081 | 0 | 0 | 0 | 0 | 0 |
| SKNSH2008043 | 0 | 18.400 | 0 | 0 | 0 | 0 | 0 |
| SMINT1000042 | 0 | 0 | 0 | 0 | 0 | 0 | 3.328 |
| SMINT1000054 | 0 | 33.993 | 0 | 0 | 0 | 0 | 0 |
| SMINT1000117 | 0 | 0 | 0 | 0 | 0 | 0 | 3.268 |
| SMINT1000131 | 3.319 | 0 | 0 | 0 | 0 | 0 | 0 |
| SMINT2000018 | 0 | 0 | 0 | 0 | 0 | 0 | 5.230 |
| SMINT2000073 | 0 | 0 | 0 | 0 | 0 | 0 | 21.554 |
| SMINT2000176 | 50.667 | 0 | 0 | 0 | 0 | 0 | 0 |
| SMINT2000232 | 0 | 0 | 0 | 0 | 0 | 0 | 17.685 |
| SMINT2000277 | 0 | 0 | 0 | 0 | 0 | 0 | 2.252 |
| SMINT2000396 | 0 | 0 | 0 | 2.950 | 0 | 0 | 3.041 |
| SMINT2000400 | 0 | 12.156 | 0 | 16.123 | 0 | 0 | 16.621 |
| SMINT2000441 | 0 | 2.461 | 0 | 1.088 | 0 | 0 | 0.561 |
| SMINT2000468 | 0 | 0 | 0 | 0 | 0 | 0 | 2.842 |
| SMINT2000537 | 0 | 0 | 0 | 0 | 0 | 0 | 26.038 |
| SMINT2000609 | 0 | 33.993 | 0 | 0 | 0 | 0 | 0 |
| SMINT2000659 | 0 | 0 | 0 | 0 | 91.616 | 0 | 0 |
| SMINT2000694 | 4.308 | 0 | 0 | 0 | 0 | 0 | 0 |
| SMINT2001615 | 0 | 0 | 0 | 40.582 | 0 | 0 | 0 |
| SMINT2002224 | 0 | 0 | 0 | 0 | 0 | 0 | 15.727 |
| SMINT2002620 | 0 | 0 | 0 | 10.921 | 0 | 0 | 0 |
| SMINT2002882 | 10.523 | 0 | 0 | 6.998 | 0 | 0 | 0 |
| SMINT2002976 | 0 | 0 | 0 | 0 | 0 | 0 | 6.480 |
| SMINT2003074 | 0 | 0 | 0 | 6.607 | 0 | 0 | 1.703 |
| SMINT2003169 | 1.705 | 0 | 0 | 1.134 | 0 | 0 | 1.169 |
| SMINT2003551 | 0 | 25.210 | 0 | 0 | 0 | 0 | 0 |
| SMINT2003905 | 2.806 | 4.221 | 0 | 0 | 0 | 0 | 0 |
| SMINT2004729 | 0 | 0 | 0 | 30.953 | 0 | 0 | 0 |
| SMINT2005368 | 8.512 | 0 | 0 | 0 | 0 | 0 | 1.459 |
| SMINT2005956 | 0 | 4.783 | 0 | 0 | 0 | 0 | 0 |
| SMINT2007187 | 0 | 12.199 | 0 | 0 | 0 | 0 | 25.019 |
| SMINT2008590 | 0 | 0 | 0 | 0 | 0 | 0 | 11.892 |
| SMINT2008672 | 0 | 0 | 0 | 32.753 | 0 | 0 | 0 |
| SMINT2008921 | 0 | 0 | 0 | 0 | 0 | 0 | 1.378 |
| SMINT2008960 | 0 | 0 | 0 | 0 | 0 | 0 | 0.850 |
| SMINT2009292 | 0 | 6.446 | 0 | 0 | 0 | 0 | 4.406 |
| SMINT2010426 | 0 | 0 | 0 | 0 | 0 | 0 | 6.730 |
| SMINT2010639 | 0 | 4.923 | 0 | 0 | 0 | 0 | 0 |
| SMINT2010901 | 0 | 0 | 0 | 8.464 | 0 | 0 | 0 |
| SMINT2012291 | 0 | 0 | 0 | 0 | 0 | 33.985 | 0 |
| SMINT2013890 | 0 | 0 | 0 | 0 | 0 | 67.622 | 0 |
| SMINT2014480 | 0 | 2.887 | 0 | 0 | 0 | 0 | 0 |
| SPLEN1000106 | 0 | 0 | 0 | 0 | 0 | 0 | 5.783 |
| SPLEN1000116 | 0 | 0 | 0 | 0 | 0 | 0 | 1.230 |
| SPLEN1000165 | 0 | 9.069 | 0 | 0 | 0 | 0 | 12.399 |
| SPLEN1000166 | 20.484 | 0 | 0 | 2.724 | 0 | 0 | 7.022 |
| SPLEN2000258 | 0 | 0 | 0 | 0 | 0 | 0 | 5.439 |
| SPLEN2000267 | 0 | 0 | 0 | 0 | 0 | 0 | 14.505 |
| SPLEN2000505 | 0 | 0 | 0 | 0 | 0 | 6.212 | 0.524 |
| SPLEN2000516 | 0 | 0 | 0 | 0 | 0 | 0 | 14.849 |
| SPLEN2000698 | 0 | 0 | 0 | 0 | 0 | 0 | 2.634 |
| SPLEN2000839 | 0 | 0 | 0 | 0 | 0 | 67.269 | 0 |
| SPLEN2000882 | 0 | 0 | 0 | 0 | 0 | 0 | 2.182 |
| SPLEN2001157 | 9.968 | 0 | 0 | 0 | 0 | 0 | 0 |
| SPLEN2002335 | 0 | 0 | 0 | 0 | 0 | 0 | 3.774 |
| SPLEN2002467 | 0 | 0 | 0 | 7.852 | 0 | 0 | 4.047 |
| SPLEN2002493 | 0 | 3.145 | 0 | 8.343 | 0 | 0 | 0 |
| SPLEN2002744 | 0 | 4.053 | 0 | 0 | 0 | 0 | 0 |
| SPLEN2004078 | 0 | 0 | 0 | 0 | 0 | 0 | 18.564 |
| SPLEN2004555 | 0 | 0 | 0 | 0 | 0 | 15.689 | 2.645 |
| SPLEN2004773 | 0 | 0 | 0 | 1.715 | 0 | 10.490 | 0 |
| SPLEN2005009 | 0 | 31.245 | 0 | 0 | 0 | 0 | 0 |
| SPLEN2005783 | 0 | 0 | 0 | 0 | 0 | 0 | 8.098 |
| SPLEN2006122 | 0 | 1.530 | 0 | 0 | 0 | 0 | 0 |
| SPLEN2006133 | 0 | 0 | 0 | 0 | 0 | 0 | 1.178 |
| SPLEN2006143 | 0 | 0 | 0 | 0 | 0 | 0 | 2.779 |
| SPLEN2006305 | 0 | 2.296 | 0 | 1.522 | 0 | 0 | 2.354 |
| SPLEN2006325 | 30.193 | 0 | 0 | 0 | 0 | 0 | 0 |
| SPLEN2006374 | 5.509 | 0 | 0 | 0 | 0 | 0 | 3.776 |
| SPLEN2007182 | 0 | 0 | 0 | 0 | 0 | 0 | 15.878 |
| SPLEN2007739 | 0 | 0 | 0 | 0 | 0 | 0 | 7.543 |
| SPLEN2008460 | 0 | 0 | 0 | 0 | 0 | 0 | 1.627 |
| SPLEN2009555 | 0 | 15.436 | 0 | 0 | 0 | 0 | 10.552 |
| SPLEN2009970 | 0 | 0 | 0 | 0 | 0 | 37.585 | 12.671 |
| SPLEN2010195 | 0 | 0.705 | 0 | 1.870 | 0 | 0 | 0.964 |
| SPLEN2010912 | 0 | 0 | 0 | 0 | 0 | 0 | 15.386 |
| SPLEN2011737 | 0 | 0 | 0 | 0 | 0 | 0 | 3.237 |
| SPLEN2011758 | 19.034 | 0 | 0 | 0 | 0 | 0 | 6.525 |
| SPLEN2012571 | 0 | 5.777 | 0 | 15.323 | 0 | 0 | 0 |
| SPLEN2014080 | 0 | 8.610 | 0 | 0 | 0 | 0 | 11.772 |
| SPLEN2014911 | 0 | 0 | 0 | 0 | 0 | 7.775 | 0 |
| SPLEN2015310 | 0 | 1.042 | 0 | 0 | 0 | 0 | 0 |
| SPLEN2016268 | 6.263 | 2.355 | 12.432 | 0 | 0 | 3.184 | 2.147 |
| SPLEN2016972 | 7.349 | 0 | 0 | 0 | 0 | 0 | 0 |
| SPLEN2017318 | 0 | 0 | 0 | 0 | 0 | 0 | 6.304 |
| SPLEN2017351 | 0 | 10.932 | 0 | 7.249 | 0 | 0 | 7.473 |
| SPLEN2018157 | 0 | 0 | 0 | 5.813 | 0 | 0 | 14.982 |
| SPLEN2018159 | 0 | 17.210 | 0 | 0 | 0 | 0 | 0 |
| SPLEN2019446 | 0 | 0 | 0 | 0 | 0 | 0 | 12.927 |
| SPLEN2021122 | 0 | 0 | 0 | 0 | 0 | 0 | 20.261 |
| SPLEN2021194 | 0 | 0 | 0 | 0 | 0 | 0 | 4.191 |
| SPLEN2025017 | 0 | 0 | 0 | 0 | 0 | 0 | 41.660 |
| SPLEN2027113 | 0 | 2.974 | 0 | 7.887 | 0 | 0 | 2.033 |
| SPLEN2028466 | 1.499 | 1.503 | 0 | 0 | 0 | 9.146 | 0 |
| SPLEN2028914 | 0 | 10.149 | 0 | 6.730 | 0 | 0 | 3.469 |
| SPLEN2031547 | 0 | 2.861 | 0 | 0 | 0 | 0 | 11.735 |
| SPLEN2039379 | 0 | 0 | 0 | 0 | 0 | 0 | 10.793 |
| SPLEN2042303 | 0 | 0 | 0 | 0 | 0 | 0 | 32.557 |
| STOMA1000047 | 0 | 0 | 0 | 7.637 | 0 | 0 | 0 |
| STOMA2000121 | 4.416 | 0 | 0 | 0 | 0 | 0 | 0 |
| STOMA2000289 | 9.873 | 0 | 0 | 0 | 0 | 0 | 0 |
| STOMA2000396 | 0 | 0 | 0 | 0 | 0 | 0 | 6.402 |
| STOMA2000482 | 0 | 0 | 0 | 0 | 0 | 28.412 | 0 |
| STOMA2002166 | 0 | 2.126 | 16.835 | 0 | 0 | 0 | 0 |
| STOMA2006780 | 0 | 0 | 0 | 0 | 0 | 0 | 10.791 |
| SYNOV1000049 | 0 | 0 | 0 | 52.773 | 0 | 0 | 0 |
| SYNOV1000118 | 15.338 | 0 | 0 | 0 | 0 | 0 | 10.515 |
| SYNOV1000124 | 0 | 1.011 | 0 | 2.681 | 0 | 0 | 3.455 |
| SYNOV2001033 | 0 | 1.090 | 0 | 1.445 | 0 | 0 | 0.745 |
| SYNOV2001144 | 0 | 0 | 0 | 0 | 0 | 10.424 | 0 |
| SYNOV2001374 | 6.638 | 0 | 0 | 4.414 | 0 | 0 | 0 |
| SYNOV2005216 | 0 | 0 | 0 | 0 | 0 | 0 | 12.130 |
| SYNOV4007671 | 2.657 | 0 | 42.189 | 0 | 0 | 0 | 0 |
| SYNOV4009129 | 0 | 0 | 0 | 0 | 0 | 0 | 9.644 |
| SYNOV4009298 | 0 | 0 | 0 | 0 | 0 | 0 | 36.547 |
| T1ESE2000904 | 0 | 0 | 0 | 11.128 | 0 | 0 | 0 |
| TESOP1000035 | 0 | 0 | 0 | 0 | 0 | 0 | 5.494 |
| TESOP2002273 | 0 | 0 | 0 | 9.782 | 0 | 0 | 15.126 |
| TESOP2002451 | 0 | 0 | 0 | 0 | 0 | 0 | 5.902 |
| TESOP2007384 | 0 | 0 | 0 | 0 | 0 | 44.382 | 0 |
| TESTI1000023 | 6.079 | 0 | 0 | 0 | 0 | 0 | 2.084 |
| TESTI1000051 | 0 | 0 | 0 | 12.552 | 0 | 0 | 0 |
| TESTI1000096 | 0 | 0 | 0 | 42.162 | 0 | 0 | 0 |
| TESTI1000142 | 0 | 0 | 0 | 0 | 0 | 0 | 8.382 |
| TESTI1000266 | 0 | 0 | 0 | 0 | 0 | 0 | 65.522 |
| TESTI2000172 | 0 | 0 | 0 | 20.618 | 0 | 0 | 0 |
| TESTI2000179 | 0 | 0 | 0 | 0 | 0 | 0 | 45.508 |
| TESTI2000184 | 0 | 4.108 | 0 | 5.449 | 0 | 0 | 5.617 |
| TESTI2000598 | 0 | 0 | 0 | 12.107 | 0 | 0 | 49.922 |
| TESTI2000627 | 0 | 0 | 0 | 0 | 0 | 0 | 26.175 |
| TESTI2000784 | 0 | 0 | 0 | 16.432 | 0 | 0 | 5.647 |
| TESTI2000874 | 45.303 | 0 | 0 | 0 | 0 | 0 | 0 |
| TESTI2000970 | 0 | 0 | 0 | 0 | 0 | 0 | 12.078 |
| TESTI2001099 | 0 | 2.671 | 0 | 0 | 0 | 0 | 0.609 |
| TESTI2001269 | 0 | 0.992 | 0 | 1.316 | 0 | 4.023 | 0.678 |
| TESTI2001511 | 0 | 0 | 0 | 8.433 | 0 | 0 | 0 |
| TESTI2001613 | 0 | 0 | 0 | 0 | 0 | 0 | 16.971 |
| TESTI2001869 | 0 | 0 | 0 | 0 | 0 | 0 | 29.647 |
| TESTI2001985 | 21.274 | 0 | 0 | 0 | 0 | 0 | 7.293 |
| TESTI2002149 | 0 | 0 | 0 | 4.031 | 0 | 0 | 0 |
| TESTI2002365 | 0 | 7.938 | 0 | 2.632 | 0 | 0 | 1.357 |
| TESTI2002369 | 0 | 27.307 | 0 | 36.216 | 0 | 0 | 0 |
| TESTI2002707 | 0 | 0 | 0 | 0 | 0 | 0 | 6.175 |
| TESTI2002866 | 0 | 19.335 | 0 | 0 | 0 | 0 | 0 |
| TESTI2003127 | 0 | 0 | 0 | 0 | 0 | 0 | 13.265 |
| TESTI2003280 | 0 | 0 | 0 | 0 | 0 | 0 | 5.096 |
| TESTI2003413 | 0 | 0 | 0 | 6.275 | 0 | 0 | 3.234 |
| TESTI2003533 | 9.426 | 0 | 0 | 0 | 0 | 0 | 0 |
| TESTI2004031 | 2.906 | 0 | 0 | 0 | 0 | 0 | 2.490 |
| TESTI2004122 | 0 | 0 | 87.855 | 0 | 0 | 0 | 0 |
| TESTI2004153 | 31.445 | 0 | 0 | 10.456 | 0 | 0 | 0 |
| TESTI2004391 | 15.305 | 7.674 | 0 | 0 | 0 | 0 | 5.246 |
| TESTI2004734 | 0 | 0 | 0 | 0 | 0 | 0 | 42.599 |
| TESTI2004737 | 0 | 0 | 0 | 0 | 0 | 0 | 2.200 |
| TESTI2004929 | 10.290 | 0 | 0 | 0 | 0 | 0 | 0 |
| TESTI2005395 | 0 | 0 | 0 | 0 | 0 | 0 | 48.724 |
| TESTI2005396 | 0 | 26.815 | 0 | 35.563 | 0 | 0 | 18.331 |
| TESTI2005408 | 0 | 3.220 | 0 | 0 | 0 | 0 | 2.201 |
| TESTI2005731 | 0 | 6.004 | 0 | 0 | 0 | 0 | 0 |
| TESTI2005742 | 0 | 0 | 0 | 0 | 0 | 0 | 25.334 |
| TESTI2005743 | 0 | 0 | 0 | 0 | 0 | 0 | 2.729 |
| TESTI2005784 | 0 | 5.405 | 0 | 0 | 0 | 0 | 7.390 |
| TESTI2006120 | 0 | 17.639 | 0 | 0 | 0 | 0 | 0 |
| TESTI2006383 | 14.689 | 0 | 0 | 9.768 | 0 | 0 | 0 |
| TESTI2006588 | 1.962 | 0.984 | 0 | 0 | 0 | 0 | 1.345 |
| TESTI2006615 | 0 | 0 | 0 | 11.123 | 0 | 0 | 0 |
| TESTI2006748 | 0 | 0 | 0 | 8.291 | 0 | 0 | 4.273 |
| TESTI2006872 | 0 | 19.972 | 0 | 0 | 0 | 0 | 0 |
| TESTI2007211 | 0 | 0 | 0 | 0 | 0 | 0 | 4.103 |
| TESTI2007464 | 0 | 7.289 | 0 | 19.333 | 0 | 0 | 0 |
| TESTI2007998 | 0 | 0 | 0 | 0 | 0 | 0 | 1.964 |
| TESTI2008020 | 13.899 | 0 | 0 | 0 | 0 | 0 | 0 |
| TESTI2008240 | 0 | 0 | 0 | 0 | 0 | 1.843 | 1.243 |
| TESTI2008835 | 0 | 0 | 0 | 0 | 0 | 79.966 | 0 |
| TESTI2009388 | 72.663 | 0 | 0 | 0 | 0 | 0 | 0 |
| TESTI2009402 | 0 | 16.660 | 0 | 0 | 0 | 0 | 0 |
| TESTI2009474 | 0 | 0 | 0 | 0 | 0 | 0 | 7.530 |
| TESTI2009497 | 0 | 0 | 0 | 0 | 0 | 0 | 65.522 |
| TESTI2009520 | 0 | 0 | 0 | 3.615 | 0 | 0 | 0 |
| TESTI2009577 | 0 | 0 | 0 | 4.260 | 0 | 0 | 2.196 |
| TESTI2009785 | 5.200 | 2.607 | 0 | 6.916 | 0 | 0 | 5.347 |
| TESTI2010513 | 0 | 0 | 0 | 0 | 0 | 0 | 2.440 |
| TESTI2010617 | 0 | 0 | 0 | 16.783 | 0 | 0 | 8.651 |
| TESTI2010724 | 0 | 6.817 | 0 | 0 | 0 | 0 | 0 |
| TESTI2011680 | 0 | 13.594 | 0 | 18.029 | 0 | 0 | 18.586 |
| TESTI2011683 | 0 | 0 | 0 | 0 | 0 | 0 | 40.221 |
| TESTI2012155 | 0 | 0 | 0 | 15.444 | 0 | 0 | 7.961 |
| TESTI2013295 | 19.306 | 0 | 0 | 12.839 | 0 | 0 | 13.236 |
| TESTI2014318 | 0 | 52.242 | 0 | 0 | 0 | 0 | 0 |
| TESTI2015213 | 3.720 | 0.933 | 14.768 | 0 | 0 | 0 | 0 |
| TESTI2015246 | 0 | 0 | 0 | 28.533 | 0 | 0 | 0 |
| TESTI2015626 | 2.384 | 0 | 9.463 | 0 | 0 | 0 | 0.409 |
| TESTI2017645 | 0 | 0 | 0 | 0 | 0 | 0 | 41.342 |
| TESTI2017954 | 0 | 1.204 | 0 | 3.193 | 0 | 0 | 1.646 |
| TESTI2018014 | 0 | 0 | 0 | 9.290 | 0 | 28.407 | 0 |
| TESTI2018462 | 0 | 0 | 0 | 0 | 0 | 0 | 7.021 |
| TESTI2018867 | 0 | 0 | 0 | 0 | 0 | 20.987 | 0 |
| TESTI2020510 | 0 | 0 | 0 | 0 | 0 | 0 | 26.958 |
| TESTI2020525 | 0 | 0 | 0 | 78.664 | 0 | 0 | 0 |
| TESTI2021124 | 0 | 0 | 0 | 13.774 | 0 | 0 | 7.100 |
| TESTI2021447 | 0 | 0 | 0 | 0 | 0 | 0 | 32.208 |
| TESTI2021851 | 68.568 | 0 | 0 | 0 | 0 | 0 | 0 |
| TESTI2022179 | 0 | 19.086 | 0 | 0 | 0 | 0 | 0 |
| TESTI2023414 | 0 | 0 | 0 | 39.241 | 0 | 0 | 5.057 |
| TESTI2025757 | 0 | 0 | 0 | 40.866 | 0 | 0 | 0 |
| TESTI2026215 | 0 | 0 | 0 | 0 | 0 | 0 | 8.605 |
| TESTI2026525 | 15.768 | 0 | 0 | 0 | 0 | 0 | 0 |
| TESTI2027496 | 0 | 17.073 | 0 | 0 | 0 | 0 | 0 |
| TESTI2029227 | 0 | 0 | 0 | 14.307 | 0 | 0 | 14.749 |
| TESTI2034940 | 1.888 | 0.947 | 0 | 0 | 0 | 0 | 0 |
| TESTI2036969 | 0 | 6.529 | 0 | 0 | 0 | 0 | 8.927 |
| TESTI2039177 | 0 | 7.531 | 0 | 0 | 0 | 0 | 0 |
| TESTI2041734 | 0 | 0 | 0 | 0 | 0 | 0 | 48.724 |
| TESTI2044276 | 0 | 17.235 | 0 | 0 | 0 | 0 | 11.782 |
| TESTI2046456 | 0 | 2.523 | 0 | 5.578 | 0 | 0 | 2.300 |
| TESTI2053723 | 0 | 0 | 0 | 0 | 0 | 0 | 5.624 |
| TESTI4000014 | 0 | 0.974 | 0 | 3.443 | 0 | 2.632 | 2.440 |
| TESTI4000434 | 0 | 0 | 0 | 0 | 0 | 0 | 16.243 |
| TESTI4002072 | 0 | 6.068 | 0 | 8.048 | 0 | 0 | 4.148 |
| TESTI4002647 | 0 | 0 | 0 | 0 | 0 | 0 | 65.522 |
| TESTI4002799 | 0 | 1.605 | 0 | 4.258 | 0 | 0 | 1.646 |
| TESTI4005133 | 0 | 0 | 0 | 0 | 0 | 0 | 14.814 |
| TESTI4005399 | 0 | 0 | 0 | 0 | 0 | 0 | 9.686 |
| TESTI4006326 | 0 | 0 | 0 | 0 | 0 | 0 | 48.724 |
| TESTI4007965 | 0 | 32.797 | 0 | 0 | 0 | 0 | 0 |
| TESTI4008058 | 0 | 0 | 0 | 0 | 0 | 0 | 21.899 |
| TESTI4009286 | 0 | 0 | 0 | 1.747 | 0 | 0 | 0 |
| TESTI4009454 | 14.566 | 0 | 0 | 9.687 | 0 | 0 | 0 |
| TESTI4010851 | 0 | 1.987 | 0 | 5.270 | 0 | 0 | 4.075 |
| TESTI4012960 | 0 | 0 | 0 | 71.770 | 0 | 0 | 18.497 |
| TESTI4013441 | 0.568 | 0.285 | 0 | 0.378 | 0 | 1.156 | 1.169 |
| TESTI4014175 | 0 | 0 | 0 | 0 | 0 | 0 | 33.186 |
| TESTI4015442 | 0 | 0 | 0 | 48.593 | 0 | 0 | 25.047 |
| TESTI4017714 | 0 | 0 | 0 | 0 | 0 | 0 | 48.724 |
| TESTI4018436 | 0 | 9.457 | 0 | 0 | 0 | 0 | 0 |
| TESTI4020596 | 0 | 0 | 0 | 0 | 0 | 0 | 65.522 |
| TESTI4024740 | 0 | 0 | 0 | 43.356 | 0 | 0 | 0 |
| TESTI4029671 | 0 | 0 | 0 | 44.826 | 0 | 0 | 0 |
| TESTI4032913 | 0 | 0 | 0 | 0 | 0 | 59.736 | 0 |
| TESTI4035770 | 0 | 0 | 0 | 0 | 0 | 0 | 65.522 |
| TESTI4037156 | 2.006 | 1.509 | 0 | 4.669 | 0 | 0 | 0.688 |
| TESTI4038284 | 0 | 3.347 | 0 | 8.877 | 0 | 0 | 4.576 |
| TESTI4043140 | 0 | 0 | 0 | 0 | 0 | 0 | 3.445 |
| TESTI4043223 | 0 | 0 | 0 | 0 | 0 | 0 | 34.381 |
| THYMU1000002 | 0 | 1.076 | 0 | 1.427 | 0 | 4.362 | 0 |
| THYMU1000016 | 0 | 0.702 | 0 | 1.863 | 0 | 0 | 0.480 |
| THYMU1000041 | 0 | 0 | 0 | 0 | 0 | 17.525 | 0 |
| THYMU1000083 | 0 | 5.449 | 0 | 0 | 0 | 22.099 | 7.450 |
| THYMU1000103 | 0 | 6.408 | 0 | 4.249 | 0 | 0 | 0 |
| THYMU1000144 | 0 | 0 | 0 | 0 | 0 | 0 | 11.247 |
| THYMU1000329 | 19.315 | 0 | 0 | 0 | 0 | 0 | 0 |
| THYMU1000374 | 0 | 0 | 0 | 7.220 | 0 | 0 | 1.861 |
| THYMU1000428 | 0 | 0 | 0 | 1.370 | 0 | 0 | 0 |
| THYMU1000499 | 20.685 | 0 | 0 | 0 | 0 | 0 | 0 |
| THYMU2000369 | 0 | 0 | 0 | 0 | 0 | 0 | 22.334 |
| THYMU2000436 | 3.339 | 0 | 0 | 0 | 0 | 0 | 0 |
| THYMU2000702 | 0 | 11.372 | 0 | 0 | 0 | 0 | 0 |
| THYMU2000946 | 5.243 | 5.258 | 0 | 3.487 | 0 | 0 | 3.595 |
| THYMU2001007 | 0 | 0 | 0 | 0 | 0 | 19.994 | 0 |
| THYMU2001926 | 0 | 35.271 | 0 | 0 | 0 | 0 | 0 |
| THYMU2002583 | 0 | 0 | 0 | 1.249 | 0 | 0 | 0 |
| THYMU2002841 | 0 | 5.274 | 0 | 0 | 0 | 0 | 0 |
| THYMU2003046 | 0 | 0 | 0 | 0 | 0 | 14.168 | 0 |
| THYMU2003419 | 1.820 | 0 | 0 | 1.210 | 0 | 0 | 0 |
| THYMU2003446 | 0 | 4.334 | 0 | 0 | 0 | 0 | 0 |
| THYMU2003981 | 0 | 0 | 0 | 0 | 0 | 0 | 0.649 |
| THYMU2004152 | 0 | 0 | 0 | 0 | 0 | 0 | 1.233 |
| THYMU2004344 | 4.630 | 2.321 | 0 | 6.158 | 0 | 0 | 6.348 |
| THYMU2004916 | 0 | 0 | 0 | 3.761 | 0 | 0 | 0 |
| THYMU2005546 | 0 | 0 | 0 | 0 | 0 | 0 | 2.718 |
| THYMU2006505 | 0 | 0 | 0 | 0 | 0 | 0 | 6.783 |
| THYMU2006813 | 0 | 0 | 0 | 0 | 0 | 0 | 3.931 |
| THYMU2007036 | 0 | 0 | 0 | 0 | 0 | 0 | 19.184 |
| THYMU2007065 | 14.696 | 0 | 0 | 0 | 0 | 0 | 0 |
| THYMU2007112 | 0 | 0 | 0 | 0 | 0 | 0 | 11.363 |
| THYMU2007339 | 0 | 0 | 0 | 74.295 | 0 | 0 | 0 |
| THYMU2007694 | 0 | 0 | 0 | 0 | 0 | 0 | 25.756 |
| THYMU2007886 | 0 | 0 | 0 | 3.337 | 0 | 0 | 0 |
| THYMU2008049 | 0 | 0 | 0 | 0 | 0 | 0 | 56.118 |
| THYMU2008321 | 20.953 | 0 | 0 | 0 | 0 | 0 | 3.591 |
| THYMU2009592 | 0 | 0 | 0 | 0 | 0 | 0 | 34.320 |
| THYMU2010448 | 0 | 19.245 | 0 | 0 | 0 | 0 | 6.578 |
| THYMU2011482 | 0 | 0 | 0 | 0 | 0 | 0 | 3.813 |
| THYMU2011875 | 81.295 | 0 | 0 | 0 | 0 | 0 | 0 |
| THYMU2011881 | 0 | 0 | 0 | 0 | 0 | 0 | 12.950 |
| THYMU2012002 | 0 | 0 | 0 | 0 | 0 | 0 | 31.541 |
| THYMU2012024 | 0 | 0 | 0 | 0 | 0 | 0 | 3.702 |
| THYMU2012625 | 0 | 0 | 0 | 0 | 0 | 7.656 | 0 |
| THYMU2013136 | 0 | 2.973 | 0 | 0 | 0 | 0 | 2.032 |
| THYMU2013916 | 0 | 2.016 | 0 | 0 | 0 | 0 | 0 |
| THYMU2014167 | 0 | 0 | 0 | 0 | 0 | 0 | 20.480 |
| THYMU2015762 | 0 | 0 | 0 | 0 | 0 | 0 | 2.463 |
| THYMU2016204 | 0 | 0 | 0 | 0 | 0 | 0 | 59.836 |
| THYMU2017158 | 0 | 0 | 0 | 0 | 0 | 0 | 59.836 |
| THYMU2019021 | 0 | 0 | 0 | 11.713 | 0 | 0 | 0 |
| THYMU2025939 | 0 | 0 | 0 | 0 | 0 | 0 | 19.812 |
| THYMU2026350 | 0 | 0 | 0 | 0 | 0 | 0 | 59.836 |
| THYMU2027125 | 1.297 | 0.651 | 0 | 0.431 | 0 | 0 | 0 |
| THYMU2031341 | 0 | 0 | 0 | 0 | 0 | 0 | 4.286 |
| THYMU2032825 | 0 | 0 | 0 | 0 | 0 | 0 | 11.550 |
| THYMU2034917 | 0 | 0 | 0 | 46.831 | 0 | 0 | 0 |
| THYMU3001939 | 0 | 0 | 0 | 0 | 0 | 0 | 59.836 |
| THYMU3002578 | 0 | 0 | 0 | 0 | 0 | 0 | 38.004 |
| THYMU3003309 | 0 | 0 | 0 | 0 | 0 | 0 | 36.117 |
| THYMU3008105 | 0 | 0 | 0 | 0 | 0 | 0 | 25.159 |
| THYMU3011827 | 0 | 0 | 0 | 0 | 0 | 0 | 28.855 |
| THYMU3015759 | 0 | 4.092 | 0 | 0 | 0 | 0 | 1.399 |
| THYMU3026000 | 0 | 0 | 0 | 0 | 0 | 30.477 | |
| THYMU3032798 | 0 | 0 | 0 | 0 | 0 | 0 | 4.804 |
| THYMU3033626 | 0 | 0 | 0 | 0 | 0 | 0 | 6.982 |
| THYMU3034671 | 0 | 0 | 0 | 0 | 0 | 0 | 5.926 |
| THYMU3041428 | 0 | 0.463 | 0 | 0.614 | 0 | 0 | 0 |
| THYMU3047115 | 0 | 0.747 | 0 | 0 | 0 | 57.541 | 0 |
| TKIDN1000044 | 0 | 0 | 0 | 0 | 0 | 0 | 11.633 |
| TKIDN1000192 | 0 | 0 | 0 | 13.315 | 0 | 0 | 6.863 |
| TKIDN2000127 | 0 | 32.928 | 0 | 0 | 0 | 0 | 0 |
| TKIDN2000246 | 0 | 1.106 | 0 | 0 | 0 | 0 | 0 |
| TKIDN2001529 | 0 | 0 | 0 | 0 | 0 | 0 | 25.127 |
| TKIDN2002738 | 0 | 0 | 0 | 0 | 0 | 3.091 | 0 |
| TKIDN2003078 | 0 | 0 | 0 | 33.378 | 0 | 0 | 0 |
| TKIDN2005487 | 0 | 0 | 0 | 0 | 0 | 0 | 1.785 |
| TKIDN2009641 | 0 | 0 | 0 | 0 | 0 | 0 | 7.182 |
| TKIDN2010812 | 0 | 0 | 0 | 0 | 0 | 0 | 13.244 |
| TKIDN2010934 | 0 | 0 | 0 | 0 | 0 | 0 | 25.127 |
| TKIDN2011289 | 0 | 0 | 0 | 14.408 | 0 | 0 | 0 |
| TKIDN2013287 | 0 | 0 | 0 | 0 | 0 | 0 | 21.501 |
| TKIDN2014964 | 0 | 19.916 | 0 | 0 | 0 | 0 | 0 |
| TKIDN2015285 | 0 | 18.041 | 0 | 0 | 0 | 0 | 0 |
| TKIDN2015423 | 0 | 0 | 0 | 1.534 | 0 | 0 | 4.745 |
| TLIVE2002046 | 0 | 3.298 | 0 | 0 | 0 | 0 | 2.255 |
| TLIVE2002690 | 0 | 0 | 0 | 0 | 0 | 44.414 | 0 |
| TRACH1000030 | 2.500 | 0 | 0 | 0 | 0 | 0 | 0.857 |
| TRACH1000106 | 43.031 | 0 | 0 | 0 | 0 | 0 | 0 |
| TRACH1000181 | 0 | 0 | 0 | 0 | 0 | 34.915 | 5.885 |
| TRACH1000205 | 0 | 7.152 | 0 | 0 | 0 | 0 | 4.889 |
| TRACH2000079 | 0 | 0 | 0 | 0 | 0 | 7.985 | 1.346 |
| TRACH2000237 | 0 | 9.770 | 0 | 25.916 | 0 | 0 | 0 |
| TRACH2000248 | 0 | 0 | 0 | 0 | 0 | 0 | 3.467 |
| TRACH2000321 | 1.781 | 0.893 | 0 | 1.184 | 0 | 0 | 7.324 |
| TRACH2000420 | 0 | 6.094 | 0 | 0 | 0 | 0 | 4.166 |
| TRACH2000461 | 0 | 6.231 | 0 | 8.264 | 0 | 0 | 6.390 |
| TRACH2000472 | 3.095 | 0 | 0 | 4.116 | 0 | 0 | 0 |
| TRACH2000702 | 0 | 7.134 | 0 | 9.462 | 0 | 0 | 0 |
| TRACH2000780 | 3.168 | 7.941 | 0 | 6.319 | 0 | 0 | 1.086 |
| TRACH2000920 | 0 | 0 | 0 | 7.990 | 0 | 0 | 16.474 |
| TRACH2000926 | 0 | 0 | 0 | 0 | 0 | 20.562 | 0 |
| TRACH2000959 | 0 | 0.444 | 0 | 0 | 0 | 0 | 0.607 |
| TRACH2001021 | 0 | 0.761 | 0 | 1.009 | 0 | 0 | 0 |
| TRACH2001109 | 0 | 0.380 | 0 | 1.009 | 0 | 0 | 1.560 |
| TRACH2001289 | 1.232 | 2.471 | 0 | 2.458 | 0 | 0 | 2.956 |
| TRACH2001314 | 0 | 0 | 0 | 17.621 | 0 | 0 | 0 |
| TRACH2001463 | 0 | 11.128 | 0 | 7.379 | 0 | 0 | 3.804 |
| TRACH2001478 | 0 | 1.055 | 0 | 0 | 0 | 0 | 0.721 |
| TRACH2001607 | 0 | 0 | 0 | 0 | 0 | 0 | 1.248 |
| TRACH2001612 | 0 | 7.259 | 0 | 9.627 | 0 | 0 | 0 |
| TRACH2001810 | 0 | 1.411 | 0 | 1.872 | 0 | 0 | 2.894 |
| TRACH2001898 | 0 | 0 | 0 | 2.457 | 0 | 0 | 1.266 |
| TRACH2002537 | 0 | 0 | 0 | 0 | 0 | 0 | 2.304 |
| TRACH2002803 | 0 | 0 | 0 | 0 | 0 | 0 | 2.589 |
| TRACH2003272 | 0 | 0 | 0 | 0 | 0 | 0 | 9.205 |
| TRACH2003323 | 0 | 0 | 0 | 0 | 0 | 13.116 | 4.422 |
| TRACH2003347 | 5.758 | 0 | 0 | 3.829 | 0 | 0 | 3.947 |
| TRACH2005800 | 0 | 0 | 0 | 2.899 | 0 | 0 | 0 |
| TRACH2005811 | 3.822 | 0 | 0 | 0 | 0 | 0 | 0 |
| TRACH2007358 | 0 | 0 | 0 | 0 | 0 | 86.807 | 0 |
| TRACH2007399 | 0 | 1.712 | 0 | 0 | 0 | 0 | 0.585 |
| TRACH2008081 | 0 | 0 | 0 | 0 | 0 | 0 | 1.587 |
| TRACH2008130 | 0 | 0 | 0 | 0 | 0 | 0 | 52.585 |
| TRACH2008300 | 0 | 0 | 0 | 0 | 0 | 0 | 6.327 |
| TRACH2008472 | 0 | 5.703 | 0 | 0 | 0 | 0 | 3.898 |
| TRACH2010771 | 0 | 61.866 | 0 | 0 | 0 | 0 | 0 |
| TRACH2011290 | 0 | 0 | 0 | 0 | 0 | 0 | 52.585 |
| TRACH2011894 | 0 | 16.160 | 0 | 0 | 0 | 0 | 0 |
| TRACH2012298 | 0 | 2.579 | 0 | 3.420 | 0 | 0 | 0 |
| TRACH2013123 | 0 | 21.855 | 0 | 28.985 | 0 | 0 | 0 |
| TRACH2013552 | 0 | 0 | 0 | 0 | 0 | 0 | 12.906 |
| TRACH2014124 | 0 | 0 | 0 | 0 | 0 | 0 | 2.565 |
| TRACH2015823 | 0 | 7.950 | 0 | 0 | 0 | 0 | 5.435 |
| TRACH2018446 | 0 | 0 | 0 | 0 | 0 | 0 | 5.195 |
| TRACH2025535 | 4.208 | 3.165 | 0 | 1.399 | 0 | 0 | 0 |
| TRACH3000134 | 0 | 0 | 0 | 43.581 | 0 | 0 | 0 |
| TRACH3000692 | 0 | 0 | 0 | 11.296 | 0 | 0 | 0 |
| TRACH3002192 | 4.520 | 2.267 | 0 | 0 | 0 | 0 | 8.522 |
| TRACH3003547 | 0 | 0 | 0 | 0 | 0 | 0 | 5.253 |
| TRACH3003872 | 0 | 0 | 0 | 13.230 | 0 | 0 | 0 |
| TRACH3004424 | 0 | 30.905 | 0 | 0 | 0 | 0 | 0 |
| TRACH3004721 | 10.536 | 5.283 | 0 | 14.013 | 0 | 0 | 3.611 |
| TRACH3005211 | 0 | 0 | 0 | 44.416 | 0 | 0 | 22.894 |
| TRACH3005294 | 0 | 0 | 0 | 0 | 0 | 0 | 4.614 |
| TRACH3006717 | 0 | 6.668 | 0 | 0 | 0 | 0 | 4.558 |
| TRACH3007625 | 4.260 | 2.136 | 0 | 2.833 | 0 | 0 | 4.381 |
| TRACH3008093 | 0 | 8.902 | 0 | 0 | 0 | 0 | 9.128 |
| TRACH3008535 | 0 | 0 | 0 | 68.270 | 0 | 0 | 0 |
| TRACH3008713 | 0 | 0 | 0 | 68.270 | 0 | 0 | 0 |
| TRACH3009008 | 0 | 0 | 0 | 11.807 | 0 | 0 | 0 |
| TRACH3014316 | 0 | 0 | 0 | 0 | 0 | 0 | 52.585 |
| TRACH3016805 | 0 | 0 | 0 | 10.851 | 0 | 0 | 11.187 |
| TRACH3021544 | 0 | 0 | 0 | 0 | 0 | 0 | 52.585 |
| TRACH3026303 | 0 | 0 | 0 | 27.016 | 0 | 0 | 20.888 |
| TRACH3028855 | 0 | 0 | 0 | 0 | 0 | 0 | 27.714 |
| TRACH3037067 | 0 | 2.921 | 0 | 0 | 0 | 0 | 0 |
| TUTER1000122 | 0 | 0 | 0 | 0 | 0 | 0 | 8.109 |
| TUTER2000057 | 0 | 7.539 | 0 | 0 | 0 | 0 | 0 |
| UMVEN2000152 | 0 | 0 | 0 | 0 | 0 | 0 | 2.935 |
| UMVEN2000354 | 0 | 0 | 0 | 1.985 | 0 | 0 | 0 |
| UMVEN2000453 | 0 | 0 | 0 | 1.258 | 0 | 3.848 | 0 |
| UTERU1000106 | 0 | 10.472 | 0 | 0 | 0 | 0 | 0 |
| UTERU1000182 | 0 | 2.948 | 0 | 0 | 0 | 0 | 0 |
| UTERU1000384 | 0 | 12.952 | 0 | 0 | 0 | 0 | 0 |
| UTERU2000095 | 0 | 2.256 | 0 | 0 | 0 | 3.050 | 1.028 |
| UTERU2000263 | 0 | 0.931 | 0 | 1.235 | 0 | 0 | 0 |
| UTERU2000300 | 0 | 0 | 0 | 0 | 0 | 0 | 6.127 |
| UTERU2000424 | 0 | 0 | 0 | 0 | 0 | 0 | 1.055 |
| UTERU2000696 | 0 | 0 | 0 | 6.858 | 0 | 0 | 0 |
| UTERU2001607 | 0 | 2.681 | 0 | 0 | 0 | 0 | 1.833 |
| UTERU2002294 | 0 | 9.896 | 0 | 0 | 0 | 0 | 0 |
| UTERU2002410 | 0 | 1.067 | 0 | 0 | 0 | 0 | 0 |
| UTERU2002662 | 6.448 | 3.233 | 0 | 12.865 | 0 | 0 | 2.210 |
| UTERU2003411 | 0 | 0 | 0 | 0 | 99.640 | 0 | 0 |
| UTERU2004197 | 0 | 7.308 | 0 | 0 | 0 | 0 | 2.498 |
| UTERU2004807 | 0 | 0 | 0 | 0 | 0 | 7.404 | 0 |
| UTERU2005354 | 7.928 | 3.975 | 0 | 5.272 | 0 | 0 | 2.717 |
| UTERU2006593 | 0 | 8.050 | 0 | 10.676 | 0 | 0 | 0 |
| UTERU2007924 | 9.693 | 0 | 0 | 0 | 0 | 0 | 0 |
| UTERU2008085 | 2.291 | 0.164 | 2.599 | 0.435 | 0 | 0 | 0.112 |
| UTERU2008302 | 0 | 0 | 0 | 3.220 | 0 | 9.846 | 0 |
| UTERU2008653 | 0 | 0 | 0 | 0 | 0 | 0 | 51.019 |
| UTERU2009335 | 10.992 | 0 | 0 | 0 | 0 | 0 | 3.768 |
| UTERU2009435 | 0 | 0 | 0 | 12.888 | 0 | 0 | 0 |
| UTERU2009972 | 0 | 0 | 0 | 0 | 0 | 0 | 39.610 |
| UTERU2010226 | 28.588 | 0 | 0 | 0 | 0 | 0 | 0 |
| UTERU2010417 | 1.143 | 1.146 | 0 | 3.799 | 0 | 4.647 | 1.567 |
| UTERU2011621 | 0 | 0 | 0 | 0 | 0 | 0 | 12.335 |
| UTERU2012230 | 0 | 3.198 | 0 | 4.241 | 0 | 0 | 0 |
| UTERU2012286 | 9.182 | 9.208 | 0 | 18.318 | 0 | 0 | 0 |
| UTERU2012526 | 0 | 0 | 0 | 22.255 | 0 | 0 | 0 |
| UTERU2012767 | 0 | 0 | 0 | 25.668 | 0 | 0 | 6.615 |
| UTERU2012938 | 0 | 0 | 0 | 0 | 0 | 0 | 51.019 |
| UTERU2013262 | 0 | 0 | 0 | 44.229 | 0 | 0 | 0 |
| UTERU2013491 | 0 | 0 | 0 | 0 | 0 | 86.071 | 0 |
| UTERU2013586 | 0 | 0 | 0 | 0 | 0 | 0 | 8.185 |
| UTERU2014167 | 0 | 1.656 | 0 | 0 | 0 | 0 | 0 |
| UTERU2014678 | 0 | 0 | 0 | 0 | 0 | 0 | 51.019 |
| UTERU2014898 | 5.514 | 0 | 0 | 0 | 0 | 0 | 0 |
| UTERU2015653 | 0 | 1.060 | 0 | 0 | 0 | 4.300 | 0 |
| UTERU2016426 | 0 | 0 | 0 | 30.630 | 0 | 0 | 0 |
| UTERU2016669 | 29.924 | 0 | 0 | 0 | 0 | 0 | 0 |
| UTERU2016799 | 0 | 0 | 0 | 0 | 0 | 0 | 7.224 |
| UTERU2018884 | 0 | 0 | 0 | 0 | 0 | 0 | 1.925 |
| UTERU2019005 | 0 | 0 | 0 | 0 | 0 | 0 | 8.023 |
| UTERU2019096 | 16.696 | 4.186 | 0 | 0 | 0 | 0 | 11.447 |
| UTERU2023175 | 5.396 | 0 | 0 | 7.176 | 0 | 0 | 1.850 |
| UTERU2024969 | 0 | 0 | 0 | 7.740 | 0 | 0 | 0 |
| UTERU2028734 | 0 | 0 | 0 | 0 | 0 | 0 | 27.272 |
| UTERU3000727 | 0 | 41.026 | 0 | 0 | 0 | 0 | 0 |
| UTERU3000899 | 0 | 0 | 0 | 0 | 0 | 0 | 35.054 |
| UTERU3003660 | 0 | 0 | 0 | 17.745 | 0 | 0 | 9.147 |
| UTERU3004418 | 0 | 0 | 0 | 0 | 0 | 0 | 36.166 |
| UTERU3010409 | 0 | 0 | 0 | 0 | 0 | 0 | 8.599 |

Genes whose expression levels were negligibly low in each of the above-described libraries are shown with their clone names below.

3NB691000018, 3NB691000085, 3NB691000113, 3NB691000191, 3NB692000012, 3NB692000029, 3NB692000102, 3NB692000154, 3NB692000276, 3NB692000305, 3NB692000330, 3NB692000429, 3NB692000484, 3NB692000529, 3NB692000973, 3NB692001034, 3NB692001040, 3NB692001123, 3NB692001267, 3NB692001339, 3NB692001349, 3NB692001366, 3NB692001408, 3NB692001433, 3NB692001459, 3NB692001471, 3NB692001501, 3NB692001507, 3NB692001511, 3NB692001519, 3NB692001520, 3NB692001538, 3NB692001548, 3NB692001557, 3NB692001637, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064,

ADIPS2000069, ADIPS2000088, ADIPS2000245, ADRGL1000018, ADRGL1000033, ADRGL1000038, ADRGL1000067, ADRGL1000111, ADRGL1000147, ADRGL1000160, ADRGL1000165, ADRGL1000182, ADRGL2000056, ADRGL2000064, ADRGL2000074, ADRGL2000085, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002753, ADRGL2002857, ADRGL2003017, ADRGL2003329, ADRGL2003552,

ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146,

ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, AHMSC1000138, ASTRO1000009, ASTRO1000018, ASTRO1000029, ASTRO1000096, ASTRO2000011, ASTRO2000014, ASTRO2000095, ASTRO2000141, ASTRO2000191, ASTRO2000278, ASTRO2000372, ASTRO2000403, ASTRO2000417, ASTRO2000482, ASTRO2000533, ASTRO2000653, ASTRO2000662, ASTRO2000725, ASTRO2001001, ASTRO2001008, ASTRO2001088, ASTRO2001122, ASTRO2001227, ASTRO2001249, ASTRO2001480, ASTRO2001806, ASTRO2002024, ASTRO2002035, ASTRO2002064, ASTRO2002459, ASTRO2002571, ASTRO2002632, ASTRO2002659, ASTRO2002720, ASTRO2002733, ASTRO2002743,

ASTRO2002842, ASTRO2003024, ASTRO2003212, ASTRO2003574, ASTRO2003581, ASTRO2003740, ASTRO2003840, ASTRO2003925, ASTRO2003960, ASTRO2004051, ASTRO2004177, ASTRO2004217, ASTRO2004297, ASTRO2004584, ASTRO2004628, ASTRO2004751, ASTRO2004877, ASTRO2005008, ASTRO2005081, ASTRO2005096, ASTRO2005242, ASTRO2005273, ASTRO2005343, ASTRO2005360, ASTRO2005413, ASTRO2005557, ASTRO2005575, ASTRO2005593, ASTRO2005726, ASTRO2005863, ASTRO2005896, ASTRO2005902, ASTRO2006356, ASTRO2006475, ASTRO2006732, ASTRO2006747, ASTRO2006920, ASTRO2006952, ASTRO2007047, ASTRO2007221, ASTRO2007377, ASTRO2007515, ASTRO2007578, ASTRO2007643, ASTRO2007666, ASTRO2007948, ASTRO2007956, ASTRO2007962, ASTRO2008040, ASTRO2008239,

ASTRO2008425, ASTRO2008508, ASTRO2008777, ASTRO2008895, ASTRO2008960, ASTRO2008972, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2011149, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014863, ASTRO2014923, ASTRO2014961, ASTRO2015162, ASTRO2015185, ASTRO2015328, ASTRO2015987, ASTRO2016044, ASTRO2016114, ASTRO2016681, ASTRO2016847, ASTRO2016892, ASTRO2017348, ASTRO2017627, ASTRO2018169, ASTRO2018373, ASTRO2018540,

ASTRO2019039, ASTRO2019171, ASTRO3000154, ASTRO3000172, ASTRO3000177, ASTRO3000301, ASTRO3000474, ASTRO3000482, BEAST2000454, BGGI11000123, BGGI11000193, BGGI11000285, BGGI12000022, BGGI12000067, BGGI12000161, BGGI12000544, BGGI12000693, BGGI12001241, BGGI12001247, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972,

BLADE2003474, BLADE2003916, BLADE2004345, BLADE2004389, BLADE2004462, BLADE2004628, BLADE2004670, BLADE2004722, BLADE2005036, BLADE2005038, BLADE2005229, BLADE2005459, BLADE2005792, BLADE2006043, BLADE2006412, BLADE2006415, BLADE2006607, BLADE2006826, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008479, BLADE2008539, BLADE2008809, BLADE2008995, BLADE2009452, BLADE2009479, BLADE2009593, BNGH41000104, BNGH41000118, BNGH41000169, BNGH41000177, BNGH41000190, BNGH41000216, BNGH41000235, BNGH42000130, BNGH42000132,

BNGH42000405, BNGH42000532, BNGH42000675, BNGH42000791, BNGH42000815, BNGH42000994, BNGH42001247, BNGH42001406, BNGH42001576, BNGH42001724, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42005968, BNGH42006226, BNGH42006234, BNGH42007037, BNGH42007366, BNGH42007594, BNGH42008199, BNGH42008510, BNGH42008649, BNGH42008832, BNGH42008850, BNGH42008943, BNGH42009021, BRACE1000047, BRACE1000051, BRACE1000065, BRACE1000073, BRACE1000084, BRACE1000092, BRACE1000093, BRACE1000101, BRACE1000114, BRACE1000124, BRACE1000132, BRACE1000159, BRACE1000166,

BRACE1000169, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000331, BRACE2000351, BRACE2000368, BRACE2000392, BRACE2000441, BRACE2000489, BRACE2000505, BRACE2000526, BRACE2000531, BRACE2000545, BRACE2000553, BRACE2000565, BRACE2000577, BRACE2000640, BRACE2000650, BRACE2000677, BRACE2000718, BRACE2000743, BRACE2000750, BRACE2000753, BRACE2000815, BRACE2000864, BRACE2000885, BRACE2000893, BRACE2000905, BRACE2000936, BRACE2000947, BRACE2000965,

BRACE2000969, BRACE2000988, BRACE2001017, BRACE2001065, BRACE2001070, BRACE2001094, BRACE2001117, BRACE2001154, BRACE2001188, BRACE2001191, BRACE2001197, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001492, BRACE2001496, BRACE2001534, BRACE2001543, BRACE2001564, BRACE2001588, BRACE2001607, BRACE2001626, BRACE2001673, BRACE2001677, BRACE2001705, BRACE2001709, BRACE2001711, BRACE2001726, BRACE2001733, BRACE2001737, BRACE2001742, BRACE2001777, BRACE2001779, BRACE2001834, BRACE2001859, BRACE2001865, BRACE2001872, BRACE2001898,

BRACE2001923, BRACE2001942, BRACE2001954, BRACE2001972, BRACE2001973, BRACE2002026, BRACE2002034, BRACE2002081, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002392, BRACE2002394, BRACE2002409, BRACE2002431, BRACE2002444, BRACE2002458, BRACE2002467, BRACE2002468, BRACE2002478, BRACE2002495, BRACE2002585, BRACE2002589, BRACE2002590, BRACE2002606, BRACE2002613, BRACE2002614, BRACE2002617, BRACE2002623, BRACE2002635, BRACE2002661, BRACE2002676, BRACE2002682, BRACE2002685, BRACE2002695, BRACE2002707, BRACE2002736, BRACE2002752, BRACE2002755, BRACE2002762, BRACE2002772, BRACE2002792,

BRACE2002807, BRACE2002812, BRACE2002834, BRACE2002849, BRACE2002857, BRACE2002860, BRACE2002884, BRACE2002896, BRACE2002948, BRACE2003078, BRACE2003097, BRACE2003168, BRACE2003199, BRACE2003210, BRACE2003217, BRACE2003269, BRACE2003285, BRACE2003398, BRACE2003420, BRACE2003428, BRACE2003431, BRACE2003512, BRACE2003516, BRACE2003539, BRACE2003540, BRACE2003594, BRACE2003598, BRACE2003614, BRACE2003639, BRACE2003700, BRACE2003713, BRACE2003766, BRACE2003788, BRACE2003800, BRACE2003802, BRACE2003825, BRACE2003847, BRACE2003848, BRACE2003873, BRACE2003885, BRACE2003887, BRACE2003928, BRACE2003954, BRACE2003982, BRACE2003987, BRACE2005034, BRACE2005047, BRACE2005063, BRACE2005083, BRACE2005090,

BRACE2005099, BRACE2005118, BRACE2005124, BRACE2005138, BRACE2005160, BRACE2005169, BRACE2005193, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005363, BRACE2005408, BRACE2005433, BRACE2005448, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005731, BRACE2005742, BRACE2005762, BRACE2005773, BRACE2005787, BRACE2005795, BRACE2005800, BRACE2005858, BRACE2005869, BRACE2005881, BRACE2005898, BRACE2005904, BRACE2005906, BRACE2005907, BRACE2005911,

BRACE2005949, BRACE2005981, BRACE2005991, BRACE2006072, BRACE2006075, BRACE2006084, BRACE2006089, BRACE2006102, BRACE2006105, BRACE2006122, BRACE2006137, BRACE2006143, BRACE2006167, BRACE2006174, BRACE2006240, BRACE2006245, BRACE2006249, BRACE2006264, BRACE2006274, BRACE2006298, BRACE2006344, BRACE2006363, BRACE2006378, BRACE2006380, BRACE2006393, BRACE2006397, BRACE2006413, BRACE2006417, BRACE2006453, BRACE2006459, BRACE2006488, BRACE2006534, BRACE2006537, BRACE2006540, BRACE2006547, BRACE2006564, BRACE2006587, BRACE2006598, BRACE2006685, BRACE2006703, BRACE2006753, BRACE2006783, BRACE2006821, BRACE2006871, BRACE2006909, BRACE2006911, BRACE2006924, BRACE2006935, BRACE2006944, BRACE2006951,

BRACE2006982, BRACE2006986, BRACE2007013, BRACE2007068, BRACE2007087, BRACE2007109, BRACE2007138, BRACE2007191, BRACE2007197, BRACE2007201, BRACE2007203, BRACE2007232, BRACE2007254, BRACE2007262, BRACE2007274, BRACE2007281, BRACE2007295, BRACE2007358, BRACE2007396, BRACE2007398, BRACE2007411, BRACE2007447, BRACE2007477, BRACE2007502, BRACE2007503, BRACE2007522, BRACE2007527, BRACE2007538, BRACE2007563, BRACE2007567, BRACE2007621, BRACE2007640, BRACE2007646, BRACE2007663, BRACE2007682, BRACE2007685, BRACE2007708, BRACE2007727, BRACE2007749, BRACE2007761, BRACE2007764, BRACE2007767, BRACE2007769, BRACE2007784, BRACE2007786, BRACE2007798, BRACE2007799, BRACE2007801, BRACE2007832, BRACE2007836,

BRACE2007868, BRACE2007882, BRACE2007888, BRACE2007920, BRACE2007944, BRACE2008008, BRACE2008015, BRACE2008021, BRACE2008097, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008361, BRACE2008385, BRACE2008399, BRACE2008401, BRACE2008410, BRACE2008420, BRACE2008443, BRACE2008473, BRACE2008480, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008706, BRACE2008708, BRACE2008710, BRACE2008754, BRACE2008797, BRACE2008847, BRACE2008857, BRACE2008861, BRACE2008867, BRACE2008881,

BRACE2008883, BRACE2008917, BRACE2008919, BRACE2008941, BRACE2008948, BRACE2008960, BRACE2008999, BRACE2009014, BRACE2009016, BRACE2009031, BRACE2009044, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009293, BRACE2009311, BRACE2009318, BRACE2009323, BRACE2009366, BRACE2009421, BRACE2009428, BRACE2009434, BRACE2009437, BRACE2009483, BRACE2009517, BRACE2009533, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171,

BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012510, BRACE2012625, BRACE2012653, BRACE2012833, BRACE2012838,

BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268, BRACE2014475, BRACE2014657, BRACE2014746, BRACE2014780, BRACE2014818, BRACE2014821, BRACE2014824, BRACE2014854, BRACE2015031, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016362, BRACE2016366, BRACE2016465, BRACE2016516, BRACE2016583, BRACE2016708, BRACE2016821, BRACE2016896,

BRACE2016981, BRACE2017108, BRACE2017124, BRACE2017298, BRACE2017359, BRACE2017397, BRACE2017410, BRACE2017438, BRACE2017580, BRACE2017587, BRACE2017720, BRACE2017844, BRACE2017872, BRACE2017929, BRACE2017934, BRACE2017985, BRACE2017992, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278,

BRACE2020358, BRACE2020467, BRACE2020584, BRACE2020652, BRACE2020709, BRACE2020742, BRACE2020756, BRACE2020863, BRACE2020982, BRACE2021143, BRACE2021148, BRACE2021245, BRACE2021254, BRACE2021264, BRACE2021344, BRACE2021433, BRACE2021478, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022333, BRACE2022394, BRACE2022435, BRACE2022448, BRACE2022450, BRACE2022633, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744,

BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025710, BRACE2025796, BRACE2025992, BRACE2026131, BRACE2026141, BRACE2026142, BRACE2026293, BRACE2026294, BRACE2026404, BRACE2026675, BRACE2026836, BRACE2026920, BRACE2026971, BRACE2027018, BRACE2027221, BRACE2027312, BRACE2027382, BRACE2027389, BRACE2027408, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849,

BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035381, BRACE2035441, BRACE2035485, BRACE2036005, BRACE2036096, BRACE2036743, BRACE2036830, BRACE2036834, BRACE2036900,

BRACE2037132, BRACE2037294, BRACE2037299, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248,

BRACE2043349, BRACE2043381, BRACE2043774, BRACE2043942, BRACE2044057, BRACE2044105, BRACE2044263, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001217, BRACE3001299, BRACE3001384, BRACE3001391, BRACE3001403, BRACE3001595, BRACE3001754,

BRACE3001948, BRACE3002298, BRACE3002344, BRACE3002390, BRACE3002420, BRACE3002508, BRACE3002541, BRACE3002756, BRACE3003004, BRACE3003192, BRACE3003413, BRACE3003595, BRACE3003698, BRACE3003866, BRACE3003920, BRACE3004046, BRACE3004058, BRACE3004113, BRACE3004150, BRACE3004205, BRACE3004358, BRACE3004435, BRACE3004477, BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005989, BRACE3006113, BRACE3006185, BRACE3006226, BRACE3006462, BRACE3006463, BRACE3006553,

BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416,

BRACE3010435, BRACE3010438, BRACE3010702, BRACE3010862, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308,

BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456,

BRACE3026735, BRACE3026802, BRACE3026844, BRACE3026947, BRACE3026993, BRACE3027242, BRACE3027256, BRACE3027326, BRACE3027478, BRACE3027480, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156,

BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609,

BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696,

BRALZ2008763, BRALZ2008869, BRALZ2008930, BRALZ2009446, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000388, BRAMY2000411, BRAMY2000508, BRAMY2000585,

BRAMY2000653, BRAMY2000814, BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926,

BRAMY2003929, BRAMY2004031, BRAMY2004058, BRAMY2004183, BRAMY2004335, BRAMY2004351, BRAMY2004352, BRAMY2004387, BRAMY2004492, BRAMY2004521, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308,

BRAMY2007411, BRAMY2007458, BRAMY2007486, BRAMY2007546, BRAMY2007594, BRAMY2007610, BRAMY2007653, BRAMY2007753, BRAMY2007859, BRAMY2008333, BRAMY2008563, BRAMY2008583, BRAMY2008977, BRAMY2008979, BRAMY2009023, BRAMY2009034, BRAMY2009105, BRAMY2009123, BRAMY2009192, BRAMY2009344, BRAMY2009349, BRAMY2009394, BRAMY2009489, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208, BRAMY2010239, BRAMY2010272, BRAMY2010290, BRAMY2010366, BRAMY2010395, BRAMY2010441, BRAMY2010442, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253,

BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012119, BRAMY2012162, BRAMY2012163, BRAMY2012277, BRAMY2012340, BRAMY2012426, BRAMY2012497, BRAMY2012517, BRAMY2012536, BRAMY2012555, BRAMY2012568, BRAMY2012691, BRAMY2012721, BRAMY2012731, BRAMY2012776, BRAMY2012880, BRAMY2012881, BRAMY2013200, BRAMY2013216, BRAMY2013405, BRAMY2013414, BRAMY2013572, BRAMY2013590, BRAMY2013722, BRAMY2013736, BRAMY2013944, BRAMY2013975, BRAMY2014019, BRAMY2014387, BRAMY2014462, BRAMY2014469, BRAMY2014541, BRAMY2014754, BRAMY2014813, BRAMY2015211, BRAMY2015311, BRAMY2015420, BRAMY2015503, BRAMY2015516,

BRAMY2015550, BRAMY2015551, BRAMY2015559, BRAMY2015688, BRAMY2015782, BRAMY2015833, BRAMY2015855, BRAMY2015890, BRAMY2015911, BRAMY2015925, BRAMY2016002, BRAMY2016020, BRAMY2016070, BRAMY2016325, BRAMY2016327, BRAMY2016386, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2019055, BRAMY2019111, BRAMY2019277, BRAMY2019300, BRAMY2019468, BRAMY2019509, BRAMY2019643, BRAMY2019710, BRAMY2019985, BRAMY2019989, BRAMY2020050,

BRAMY2020058, BRAMY2020270, BRAMY2020354, BRAMY2020355, BRAMY2020412, BRAMY2020427, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172,

BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538,

BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027714, BRAMY2027717, BRAMY2027739, BRAMY2027752, BRAMY2027782, BRAMY2028072, BRAMY2028183, BRAMY2028188, BRAMY2028472, BRAMY2028491, BRAMY2028516, BRAMY2028565, BRAMY2028593, BRAMY2028682, BRAMY2028783, BRAMY2028856, BRAMY2028914, BRAMY2029204, BRAMY2029602, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442,

BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036567, BRAMY2036615, BRAMY2036699, BRAMY2036913, BRAMY2036918, BRAMY2036968,

BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760,

BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002458,

BRAMY3002508, BRAMY3002620, BRAMY3002803, BRAMY3002805, BRAMY3002886, BRAMY3003109, BRAMY3003205, BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005091, BRAMY3005656, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010492,

BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRASW1000053, BRASW1000125, BRAWH1000001, BRAWH1000002, BRAWH1000083, BRAWH1000094, BRAWH1000116, BRAWH1000130, BRAWH1000157, BRAWH1000164, BRAWH1000168, BRAWH1000174, BRAWH1000180, BRAWH1000369, BRAWH2000034, BRAWH2000048, BRAWH2000071, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000323, BRAWH2000353, BRAWH2000370, BRAWH2000409, BRAWH2000417, BRAWH2000443, BRAWH2000471,

BRAWH2000476, BRAWH2000482, BRAWH2000494, BRAWH2000500, BRAWH2000503, BRAWH2000554, BRAWH2000595, BRAWH2000643, BRAWH2000651, BRAWH2000682, BRAWH2000752, BRAWH2000859, BRAWH2000866, BRAWH2000892, BRAWH2000901, BRAWH2000926, BRAWH2000984, BRAWH2001019, BRAWH2001052, BRAWH2001103, BRAWH2001129, BRAWH2001137, BRAWH2001159, BRAWH2001203, BRAWH2001238, BRAWH2001239, BRAWH2001255, BRAWH2001355, BRAWH2001378, BRAWH2001395, BRAWH2001406, BRAWH2001418, BRAWH2001436, BRAWH2001438, BRAWH2001459, BRAWH2001461, BRAWH2001492, BRAWH2001503, BRAWH2001519, BRAWH2001535, BRAWH2001589, BRAWH2001666, BRAWH2001671, BRAWH2001679, BRAWH2001686, BRAWH2001701, BRAWH2001842, BRAWH2001873, BRAWH2001940, BRAWH2001973,

BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002601, BRAWH2002623, BRAWH2002630, BRAWH2002635, BRAWH2002662, BRAWH2002679, BRAWH2002725, BRAWH2002761, BRAWH2002861, BRAWH2002911, BRAWH2002963, BRAWH2002976, BRAWH2003000, BRAWH2003025, BRAWH2003135, BRAWH2003240, BRAWH2003307, BRAWH2003355, BRAWH2003366, BRAWH2003689, BRAWH2003832, BRAWH2003948, BRAWH2003964, BRAWH2004044, BRAWH2004068, BRAWH2004078, BRAWH2004136, BRAWH2004217, BRAWH2004580, BRAWH2004731, BRAWH2004779, BRAWH2004842, BRAWH2004884, BRAWH2004923, BRAWH2005008, BRAWH2005068, BRAWH2005074, BRAWH2005315, BRAWH2005524, BRAWH2005533,

BRAWH2005578, BRAWH2005812, BRAWH2005974, BRAWH2005998, BRAWH2006083, BRAWH2006207, BRAWH2006301, BRAWH2006395, BRAWH2006405, BRAWH2006450, BRAWH2006493, BRAWH2006526, BRAWH2006543, BRAWH2006760, BRAWH2006960, BRAWH2006989, BRAWH2006996, BRAWH2007069, BRAWH2007308, BRAWH2007406, BRAWH2007550, BRAWH2007570, BRAWH2007591, BRAWH2007658, BRAWH2007808, BRAWH2007825, BRAWH2007862, BRAWH2007890, BRAWH2008128, BRAWH2008219, BRAWH2008292, BRAWH2008297, BRAWH2008366, BRAWH2008380, BRAWH2008540, BRAWH2008577, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009304, BRAWH2009307, BRAWH2009360, BRAWH2009393,

BRAWH2009485, BRAWH2009490, BRAWH2009558, BRAWH2009590, BRAWH2009678, BRAWH2009695, BRAWH2009907, BRAWH2010000, BRAWH2010069, BRAWH2010084, BRAWH2010136, BRAWH2010318, BRAWH2010354, BRAWH2010364, BRAWH2010536, BRAWH2010552, BRAWH2010584, BRAWH2010618, BRAWH2010619, BRAWH2011066, BRAWH2011079, BRAWH2011096, BRAWH2011155, BRAWH2011166, BRAWH2011204, BRAWH2011294, BRAWH2011343, BRAWH2011400, BRAWH2011762, BRAWH2011795, BRAWH2011796, BRAWH2011812, BRAWH2011860, BRAWH2011958, BRAWH2012054, BRAWH2012077, BRAWH2012161, BRAWH2012162, BRAWH2012258, BRAWH2012277, BRAWH2012326, BRAWH2012538, BRAWH2012540, BRAWH2012619, BRAWH2012698, BRAWH2012749, BRAWH2012827, BRAWH2012963, BRAWH2013047, BRAWH2013219,

BRAWH2013294, BRAWH2013748, BRAWH2013866, BRAWH2013871, BRAWH2013914, BRAWH2013941, BRAWH2013955, BRAWH2014053, BRAWH2014234, BRAWH2014414, BRAWH2014473, BRAWH2014511, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016514, BRAWH2016562, BRAWH2016655, BRAWH2016679, BRAWH2016702, BRAWH2016724, BRAWH2016785, BRAWH2016792, BRAWH2016800, BRAWH2017103, BRAWH2017250, BRAWH2017294, BRAWH2017305, BRAWH2017379, BRAWH2017407,

BRAWH2017433, BRAWH2017523, BRAWH2017534, BRAWH2017542, BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018729, BRAWH2018745, BRAWH2018875, BRAWH2019053, BRAWH2019055, BRAWH2019198, BRAWH3000078, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3002433, BRAWH3002467, BRAWH3002513, BRAWH3002600, BRAWH3002819, BRAWH3003019, BRAWH3003136, BRAWH3003244,

BRAWH3003343, BRAWH3003411, BRAWH3003522, BRAWH3003573, BRAWH3003598, BRAWH3003801, BRAWH3003975, BRAWH3004335, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3009017, BRAWH3009285, BRAWH3009297, BRAWH3009961, BRAWH3010560, BRAWH3010602, BRAWH3010657, BRAWH3010726, BRAWH3010833, BRAWH3011101, BRAWH3011331,

BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014639, BRAWH3015017, BRAWH3015175, BRAWH3015260, BRAWH3015610, BRAWH3015825, BRAWH3016123, BRAWH3016271, BRAWH3016715, BRAWH3017260, BRAWH3017477, BRAWH3017537, BRAWH3017980, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021580, BRAWH3021641, BRAWH3021643, BRAWH3021724, BRAWH3022347, BRAWH3022431, BRAWH3022459, BRAWH3022542,

BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620,

BRAWH3032930, BRAWH3033117, BRAWH3033293, BRAWH3033448, BRAWH3033513, BRAWH3034097, BRAWH3034114, BRAWH3034134, BRAWH3034668, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000577, BRCAN2000620, BRCAN2000639, BRCAN2000763, BRCAN2000832, BRCAN2000923, BRCAN2001223,

BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203,

BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387,

BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027513, BRCAN2027593, BRCAN2027970, BRCAN2028021, BRCAN2028040, BRCAN2028319, BRCAN2028338, BRCAN2028378, BRCAN2028460, BRCAN2028545, BRCAN2028634, BRCAN2028702, BRCOC1000087, BRCOC1000094, BRCOC2000004,

BRCOC2000047, BRCOC2000186, BRCOC2000333, BRCOC2000360, BRCOC2000404, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003187, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052,

BRCOC2009196, BRCOC2009380, BRCOC2009638, BRCOC2009937, BRCOC2010115, BRCOC2010730, BRCOC2010980, BRCOC2010994, BRCOC2011010, BRCOC2011398, BRCOC2011418, BRCOC2011506, BRCOC2011769, BRCOC2011996, BRCOC2012229, BRCOC2012551, BRCOC2012813, BRCOC2013448, BRCOC2013573, BRCOC2013675, BRCOC2013780, BRCOC2014013, BRCOC2014033, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019934, BRCOC2020122, BRCOC2020142, BRHIP1000058, BRHIP1000072, BRHIP1000129, BRHIP1000258, BRHIP2000021, BRHIP2000079,

BRHIP2000087, BRHIP2000163, BRHIP2000210, BRHIP2000239, BRHIP2000245, BRHIP2000359, BRHIP2000445, BRHIP2000506, BRHIP2000532, BRHIP2000621, BRHIP2000691, BRHIP2000819, BRHIP2000826, BRHIP2000920, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719,

BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011491, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360,

BRHIP2012545, BRHIP2012580, BRHIP2012642, BRHIP2012750, BRHIP2012922, BRHIP2012923, BRHIP2012972, BRHIP2013447, BRHIP2013510, BRHIP2013699, BRHIP2013723, BRHIP2013958, BRHIP2013972, BRHIP2014063, BRHIP2014285, BRHIP2014291, BRHIP2014373, BRHIP2014386, BRHIP2014387, BRHIP2014954, BRHIP2015153, BRHIP2015356, BRHIP2015360, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018719, BRHIP2018805, BRHIP2018998, BRHIP2019007, BRHIP2019047, BRHIP2019149, BRHIP2019177,

BRHIP2019186, BRHIP2019291, BRHIP2019494, BRHIP2019589, BRHIP2019641, BRHIP2019884, BRHIP2019972, BRHIP2020182, BRHIP2020509, BRHIP2020622, BRHIP2020695, BRHIP2020743, BRHIP2020842, BRHIP2020859, BRHIP2020930, BRHIP2021102, BRHIP2021289, BRHIP2021495, BRHIP2021615, BRHIP2021744, BRHIP2021870, BRHIP2021929, BRHIP2022006, BRHIP2022054, BRHIP2022221, BRHIP2022298, BRHIP2022467, BRHIP2022708, BRHIP2023071, BRHIP2023229, BRHIP2023272, BRHIP2023292, BRHIP2023309, BRHIP2023369, BRHIP2023438, BRHIP2023695, BRHIP2023735, BRHIP2023773, BRHIP2023860, BRHIP2023869, BRHIP2024046, BRHIP2024165, BRHIP2024201, BRHIP2024347, BRHIP2024549, BRHIP2024789, BRHIP2024941, BRHIP2025245, BRHIP2025366, BRHIP2025679,

BRHIP2025739, BRHIP2025797, BRHIP2026021, BRHIP2026155, BRHIP2026214, BRHIP2026288, BRHIP2026346, BRHIP2026557, BRHIP2026723, BRHIP2026877, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP3000060, BRHIP3000070, BRHIP3000081, BRHIP3000240, BRHIP3000377, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002920, BRHIP3002931,

BRHIP3003063, BRHIP3003126, BRHIP3003306, BRHIP3003340, BRHIP3003395, BRHIP3003484, BRHIP3003644, BRHIP3003688, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006786, BRHIP3006950, BRHIP3007172, BRHIP3007183, BRHIP3007195, BRHIP3007239, BRHIP3007291, BRHIP3007409, BRHIP3007424, BRHIP3007483, BRHIP3007609, BRHIP3007640, BRHIP3007960, BRHIP3008183, BRHIP3008314, BRHIP3008344, BRHIP3008405,

BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558,

BRHIP3017637, BRHIP3017855, BRHIP3018278, BRHIP3019643, BRHIP3019824, BRHIP3019880, BRHIP3019956, BRHIP3020046, BRHIP3020155, BRHIP3020182, BRHIP3020733, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244,

BRSSN2000293, BRSSN2000295, BRSSN2000312, BRSSN2000518, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2001869, BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419,

BRSSN2008464, BRSSN2008549, BRSSN2008797, BRSSN2009119, BRSSN2009378, BRSSN2009389, BRSSN2009395, BRSSN2009627, BRSSN2009630, BRSSN2009702, BRSSN2010019, BRSSN2010110, BRSSN2010587, BRSSN2010596, BRSSN2010830, BRSSN2011262, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013874, BRSSN2014112, BRSSN2014294, BRSSN2014299, BRSSN2014424, BRSSN2014513, BRSSN2014556, BRSSN2014573, BRSSN2014636, BRSSN2014685, BRSSN2015199, BRSSN2015369, BRSSN2015493, BRSSN2015497, BRSSN2015554, BRSSN2015707, BRSSN2015773, BRSSN2015907, BRSSN2015982, BRSSN2016589,

BRSSN2016768, BRSSN2016905, BRSSN2017297, BRSSN2017422, BRSSN2017530, BRSSN2017682, BRSSN2017757, BRSSN2018218, BRSSN2018447, BRSSN2018581, BRSSN2018731, BRSSN2018771, BRSSN2018925, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475,

BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451, BRSTN2013171, BRSTN2013354, BRSTN2013502, BRSTN2013931, BRSTN2014490, BRSTN2014633, BRSTN2014751, BRSTN2014770, BRSTN2015015, BRSTN2015978, BRSTN2016066, BRSTN2016335, BRSTN2016470, BRSTN2016678, BRSTN2016892, BRSTN2016918, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021,

BRTHA1000063, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831,

BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183,

BRTHA2012189, BRTHA2012351, BRTHA2012392, BRTHA2012466, BRTHA2012555, BRTHA2012562, BRTHA2012714, BRTHA2012980, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016765, BRTHA2016918, BRTHA2017178, BRTHA2017353, BRTHA2017364, BRTHA2017972, BRTHA2017985, BRTHA2018011,

BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027229, BRTHA2027250, BRTHA2027546, BRTHA2028297, BRTHA2029969, BRTHA2030036, BRTHA2030213, BRTHA2031517,

BRTHA2031800, BRTHA2031917, BRTHA2032763, BRTHA2032845, BRTHA2033122, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000789, BRTHA3001221, BRTHA3001623, BRTHA3002265, BRTHA3002401, BRTHA3002411, BRTHA3002864, BRTHA3002933, BRTHA3002955, BRTHA3003000, BRTHA3003023, BRTHA3003074, BRTHA3003339, BRTHA3003343, BRTHA3003417, BRTHA3003449, BRTHA3003474, BRTHA3003704, BRTHA3004432, BRTHA3004475, BRTHA3004502, BRTHA3004610,

BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007532, BRTHA3007662, BRTHA3007685, BRTHA3007769, BRTHA3008143, BRTHA3008293, BRTHA3008310, BRTHA3008386, BRTHA3008520, BRTHA3008608, BRTHA3008694, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011229, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835,

BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3022641, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152,

CD34C2000175, CD34C2000443, CD34C3000125, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, COLON1000030, COLON1000084, COLON1000135, COLON2000470, COLON2000557, COLON2000568, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140,

CORDB2000061, CORDB2000127, CORDB2000541, CTONG1000009, CTONG1000020, CTONG1000022, CTONG1000040, CTONG1000056, CTONG1000062, CTONG1000086, CTONG1000088, CTONG1000093, CTONG1000097, CTONG1000113, CTONG1000137, CTONG1000165, CTONG1000175, CTONG1000180, CTONG1000181, CTONG1000220, CTONG1000277, CTONG1000283, CTONG1000302, CTONG1000341, CTONG1000467, CTONG1000476, CTONG1000488, CTONG1000508, CTONG1000540, CTONG2000031, CTONG2000042, CTONG2000201, CTONG2000218, CTONG2000279, CTONG2000303, CTONG2000330, CTONG2000395, CTONG2000411, CTONG2000465, CTONG2000480, CTONG2000561, CTONG2000589, CTONG2000766, CTONG2000771, CTONG2000819, CTONG2000827, CTONG2000846, CTONG2000855, CTONG2000977, CTONG2001139,

CTONG2001226, CTONG2001320, CTONG2001366, CTONG2001413, CTONG2001428, CTONG2001513, CTONG2001524, CTONG2001533, CTONG2001598, CTONG2001681, CTONG2001724, CTONG2001749, CTONG2001800, CTONG2001858, CTONG2001877, CTONG2001955, CTONG2002066, CTONG2002143, CTONG2002395, CTONG2002417, CTONG2002418, CTONG2002453, CTONG2002520, CTONG2002558, CTONG2002590, CTONG2002721, CTONG2002766, CTONG2002816, CTONG2002965, CTONG2002999, CTONG2003028, CTONG2003100, CTONG2003115, CTONG2003189, CTONG2003245, CTONG2003348, CTONG2003350, CTONG2003361, CTONG2003372, CTONG2003375, CTONG2003427, CTONG2003517, CTONG2003599, CTONG2003680, CTONG2003699, CTONG2003764, CTONG2003782, CTONG2003889, CTONG2003937, CTONG2003945,

CTONG2004000, CTONG2004062, CTONG2004115, CTONG2004126, CTONG2004264, CTONG2004305, CTONG2004397, CTONG2004454, CTONG2004550, CTONG2004669, CTONG2004716, CTONG2004912, CTONG2004941, CTONG2004948, CTONG2005049, CTONG2005110, CTONG2005145, CTONG2005265, CTONG2005290, CTONG2005374, CTONG2005399, CTONG2005468, CTONG2005567, CTONG2005585, CTONG2005615, CTONG2005635, CTONG2005775, CTONG2005795, CTONG2005890, CTONG2005904, CTONG2005913, CTONG2006129, CTONG2006223, CTONG2006273, CTONG2006310, CTONG2006377, CTONG2006393, CTONG2006515, CTONG2006524, CTONG2006568, CTONG2006611, CTONG2006620, CTONG2006666, CTONG2006691, CTONG2006798, CTONG2006836, CTONG2006932, CTONG2006942, CTONG2006964, CTONG2007007,

CTONG2007009, CTONG2007072, CTONG2007091, CTONG2007104, CTONG2007168, CTONG2007175, CTONG2007259, CTONG2007272, CTONG2007293, CTONG2007297, CTONG2007399, CTONG2007417, CTONG2007441, CTONG2007474, CTONG2007613, CTONG2007623, CTONG2007681, CTONG2007776, CTONG2007779, CTONG2007898, CTONG2007970, CTONG2008014, CTONG2008184, CTONG2008262, CTONG2008269, CTONG2008343, CTONG2008402, CTONG2008405, CTONG2008521, CTONG2008562, CTONG2008577, CTONG2008595, CTONG2008621, CTONG2008689, CTONG2008720, CTONG2008721, CTONG2008773, CTONG2008989, CTONG2009033, CTONG2009132, CTONG2009156, CTONG2009181, CTONG2009257, CTONG2009258, CTONG2009268, CTONG2009270, CTONG2009395, CTONG2009423, CTONG2009440, CTONG2009527,

CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010408, CTONG2010508, CTONG2010623, CTONG2010652, CTONG2010803, CTONG2011188, CTONG2011429, CTONG2011474, CTONG2011676, CTONG2011716, CTONG2011765, CTONG2011779, CTONG2011815, CTONG2011822, CTONG2011825, CTONG2011920, CTONG2011985, CTONG2012001, CTONG2012029, CTONG2012050, CTONG2012077, CTONG2012101, CTONG2012123,

CTONG2012158, CTONG2012230, CTONG2012401, CTONG2012422, CTONG2012425, CTONG2012447, CTONG2012473, CTONG2012476, CTONG2012533, CTONG2012554, CTONG2012724, CTONG2012738, CTONG2012745, CTONG2012758, CTONG2012847, CTONG2012901, CTONG2013128, CTONG2013149, CTONG2013156, CTONG2013178, CTONG2013222, CTONG2013284, CTONG2013339, CTONG2013348, CTONG2013393, CTONG2013511, CTONG2013630, CTONG2013803, CTONG2013985, CTONG2013986, CTONG2014032, CTONG2014058, CTONG2014119, CTONG2014165, CTONG2014191, CTONG2014206, CTONG2014234, CTONG2014264, CTONG2014389, CTONG2014491, CTONG2014630, CTONG2014635, CTONG2014646, CTONG2014705, CTONG2014946, CTONG2014954, CTONG2014959, CTONG2014966, CTONG2014995, CTONG2015204,

CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016185, CTONG2016217, CTONG2016355, CTONG2016499, CTONG2016505, CTONG2016559, CTONG2016658, CTONG2016775, CTONG2016797, CTONG2016846, CTONG2016869, CTONG2016904, CTONG2016942, CTONG2016953, CTONG2017021, CTONG2017094, CTONG2017292, CTONG2017409, CTONG2017429, CTONG2017458, CTONG2017604, CTONG2017650, CTONG2017798, CTONG2017804, CTONG2017814, CTONG2017910, CTONG2017939, CTONG2017989, CTONG2017998, CTONG2018062, CTONG2018069, CTONG2018147, CTONG2018217, CTONG2018279,

CTONG2018334, CTONG2018343, CTONG2018379, CTONG2018413, CTONG2018483, CTONG2018629, CTONG2018652, CTONG2018653, CTONG2018655, CTONG2018735, CTONG2018843, CTONG2018900, CTONG2018976, CTONG2019029, CTONG2019063, CTONG2019144, CTONG2019232, CTONG2019248, CTONG2019590, CTONG2019652, CTONG2019704, CTONG2019822, CTONG2019833, CTONG2020031, CTONG2020108, CTONG2020127, CTONG2020171, CTONG2020374, CTONG2020378, CTONG2020445, CTONG2020522, CTONG2020582, CTONG2020806, CTONG2020831, CTONG2020974, CTONG2021024, CTONG2021132, CTONG2021168, CTONG2021289, CTONG2022153, CTONG2022601, CTONG2022835, CTONG2023021, CTONG2023449, CTONG2023512, CTONG2024031, CTONG2024206, CTONG2024614, CTONG2024749, CTONG2025496,

CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028309, CTONG2028486, CTONG2028518, CTONG2028687, CTONG2028758, CTONG3000657, CTONG3000686, CTONG3000707, CTONG3000896, CTONG3001123, CTONG3001420, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002674, CTONG3002728, CTONG3002909, CTONG3002947, CTONG3002983, CTONG3003165, CTONG3003179, CTONG3003483, CTONG3003553,

CTONG3003598, CTONG3003652, CTONG3003654, CTONG3003669, CTONG3003737, CTONG3003972, CTONG3004072, CTONG3004317, CTONG3004397, CTONG3004550, CTONG3004576, CTONG3004607, CTONG3004712, CTONG3005325, CTONG3005648, CTONG3005713, CTONG3005803, CTONG3005813, CTONG3006067, CTONG3006073, CTONG3006186, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009227, CTONG3009287, CTONG3009328, CTONG3009385, D3OST1000066, D3OST1000109, D3OST1000238, D3OST2000184, D3OST2000618, D3OST2000654, D3OST2000734, D3OST2001328,

D3OST2001669, D3OST2001670, D3OST2001788, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000358, D6OST2000507, D9OST2000031, D9OST2000278, D9OST2000440, D9OST2000869, D9OST2001319, D9OST2001433, D9OST2002397, D9OST2002608, D9OST2002673, D9OST2003106, D9OST2003108, D9OST2003137, D9OST2003397, D9OST2003580, D9OST2003594, D9OST2003762, D9OST2003791, D9OST2003989, D9OST2004018, D9OST2004073, D9OST2004417, DFNES1000003, DFNES1000083, DFNES1000107,

DFNES1000150, DFNES1000185, DFNES2000011, DFNES2000143, DFNES2000212, DFNES2000268, DFNES2000292, DFNES2000426, DFNES2000432, DFNES2000457, DFNES2000471, DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2002966, DFNES2003081, DFNES2003192, DFNES2003255, DFNES2003742, DFNES2004354, DFNES2004383, DFNES2004684, DFNES2005266, DFNES2005527, DFNES2005540, DFNES2005779, DFNES2005875, DFNES2006944, DFNES2007113, DFNES2007299, DFNES2007332, DFNES2007641, DFNES2007757, DFNES2008280, DFNES2008881, DFNES2010502, DFNES2011192,

DFNES2011221, DFNES2011239, DFNES2011245, DFNES2011380, ERLTF1000021, ERLTF2000324, ERLTF2000465, FCBBF1000023, FCBBF1000025, FCBBF1000069, FCBBF1000117, FCBBF1000121, FCBBF1000125, FCBBF1000131, FCBBF1000155, FCBBF1000157, FCBBF1000171, FCBBF1000174, FCBBF1000182, FCBBF1000198, FCBBF1000206, FCBBF1000220, FCBBF1000232, FCBBF1000233, FCBBF1000243, FCBBF1000270, FCBBF1000272, FCBBF1000297, FCBBF1000314, FCBBF1000322, FCBBF1000349, FCBBF1000367, FCBBF1000374, FCBBF1000376, FCBBF1000395, FCBBF1000397, FCBBF1000425, FCBBF1000437, FCBBF1000506, FCBBF1000546, FCBBF1000550, FCBBF1000581, FCBBF1000582, FCBBF1000598, FCBBF1000604, FCBBF1000618, FCBBF1000627, FCBBF1000684, FCBBF1000686, FCBBF1000691,

FCBBF1000748, FCBBF2000087, FCBBF2000094, FCBBF2000105, FCBBF2000123, FCBBF2000195, FCBBF2000214, FCBBF2000232, FCBBF2000276, FCBBF2000291, FCBBF2000379, FCBBF2000473, FCBBF2000502, FCBBF2000576, FCBBF2000677, FCBBF2000678, FCBBF2000685, FCBBF2000733, FCBBF2000782, FCBBF2000808, FCBBF2000815, FCBBF2000825, FCBBF2000940, FCBBF2000951, FCBBF2001088, FCBBF2001116, FCBBF2001188, FCBBF2001238, FCBBF2001243, FCBBF2001299, FCBBF2001400, FCBBF2001427, FCBBF2001480, FCBBF2001529, FCBBF2001672, FCBBF2001675, FCBBF2001718, FCBBF2001720, FCBBF2001817, FCBBF2001868, FCBBF2002044, FCBBF2002111, FCBBF2002281, FCBBF2002370, FCBBF2002541, FCBBF2002626, FCBBF2002897, FCBBF2002898, FCBBF2002904, FCBBF2002928,

FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004090, FCBBF2004138, FCBBF2004217, FCBBF2004256, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2006131, FCBBF2006380, FCBBF2006418, FCBBF2006452, FCBBF2006634, FCBBF2007012, FCBBF2007186, FCBBF2007265, FCBBF2007610, FCBBF2007633, FCBBF3000120, FCBBF3000175, FCBBF3000227, FCBBF3000228, FCBBF3000229, FCBBF3000233, FCBBF3000269, FCBBF3000361, FCBBF3000367, FCBBF3000462,

FCBBF3000473, FCBBF3000501, FCBBF3000518, FCBBF3000536, FCBBF3000550, FCBBF3000847, FCBBF3001018, FCBBF3001020, FCBBF3001235, FCBBF3001302, FCBBF3001470, FCBBF3001594, FCBBF3001912, FCBBF3001914, FCBBF3001918, FCBBF3001924, FCBBF3002163, FCBBF3002188, FCBBF3002190, FCBBF3002268, FCBBF3002475, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3004021, FCBBF3004041, FCBBF3004390, FCBBF3004502, FCBBF3004842, FCBBF3004847, FCBBF3005160, FCBBF3005218, FCBBF3005444, FCBBF3005698, FCBBF3005729, FCBBF3006171, FCBBF3006249, FCBBF3006288, FCBBF3006399, FCBBF3006628, FCBBF3007077,

FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007631, FCBBF3007829, FCBBF3007855, FCBBF3007860, FCBBF3008073, FCBBF3008153, FCBBF3008159, FCBBF3008251, FCBBF3008311, FCBBF3008362, FCBBF3008475, FCBBF3008556, FCBBF3008689, FCBBF3008748, FCBBF3008870, FCBBF3008938, FCBBF3008944, FCBBF3009101, FCBBF3009256, FCBBF3009317, FCBBF3009428, FCBBF3009464, FCBBF3009526, FCBBF3009717, FCBBF3009888, FCBBF3009949, FCBBF3009978, FCBBF3010008, FCBBF3010124, FCBBF3010130, FCBBF3010149, FCBBF3010211, FCBBF3010361, FCBBF3010508, FCBBF3010544, FCBBF3010586, FCBBF3010601, FCBBF3010665, FCBBF3010729, FCBBF3010733,

FCBBF3011003, FCBBF3011418, FCBBF3011523, FCBBF3011570, FCBBF3011592, FCBBF3011867, FCBBF3011889, FCBBF3012170, FCBBF3012182, FCBBF3012288, FCBBF3012332, FCBBF3012588, FCBBF3012842, FCBBF3012901, FCBBF3013041, FCBBF3013058, FCBBF3013205, FCBBF3013266, FCBBF3013307, FCBBF3013341, FCBBF3013506, FCBBF3013623, FCBBF3013800, FCBBF3013846, FCBBF3014229, FCBBF3014355, FCBBF3014567, FCBBF3015119, FCBBF3015131, FCBBF3015317, FCBBF3015727, FCBBF3015758, FCBBF3015809, FCBBF3015870, FCBBF3016018, FCBBF3016178, FCBBF3016618, FCBBF3016622, FCBBF3016644, FCBBF3016987, FCBBF3017059, FCBBF3017071, FCBBF3017123, FCBBF3017233, FCBBF3017255, FCBBF3017531, FCBBF3017535, FCBBF3017681, FCBBF3017729, FCBBF3017873,

FCBBF3017974, FCBBF3018067, FCBBF3018173, FCBBF3018591, FCBBF3018753, FCBBF3018796, FCBBF3019085, FCBBF3019320, FCBBF3019564, FCBBF3019569, FCBBF3019570, FCBBF3019663, FCBBF3019716, FCBBF3019839, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022566, FCBBF3022765, FCBBF3022767, FCBBF3022894, FCBBF3023061, FCBBF3023078, FCBBF3023372, FCBBF3023443, FCBBF3023599, FCBBF3023704, FCBBF3023895, FCBBF3024096, FCBBF3024225, FCBBF3024266, FCBBF3024364, FCBBF3024793,

FCBBF3024911, FCBBF3025098, FCBBF3025252, FCBBF3025280, FCBBF3025285, FCBBF3025417, FCBBF3025568, FCBBF3025650, FCBBF3025674, FCBBF3025905, FCBBF3026021, FCBBF3026251, FCBBF3026308, FCBBF3026678, FCBBF3026692, FCBBF3027104, FCBBF3027199, FCBBF3027328, FCBBF3027559, FCBBF3027717, FCBBF3027755, FCBBF3027768, FCBBF3027863, FCBBF3027903, FCBBF3028102, FCBBF3028143, FCBBF3028188, FCBBF3028202, FCBBF3028472, FCBBF3028528, FCBBF3028582, FCBBF3028671, FCBBF3028794, FCBBF4000061, FCBBF4000076, FCBBF4000173, FCBBF4000192, FCBBF4000268, FCBBF4000282, FCBBF4000399, FCBBF4000415, FCBBF4000500, FCBBF4000529, FCBBF4000548, FCBBF5000041, FCBBF5000261, FCBBF5000325, FCBBF5000353, FCBBF5000384, FCBBF5000483,

FCBBF5000495, FEBRA1000005, FEBRA1000022, FEBRA1000138, FEBRA1000148, FEBRA1000149, FEBRA1000183, FEBRA1000189, FEBRA1000190, FEBRA2000007, FEBRA2000021, FEBRA2000035, FEBRA2000053, FEBRA2000126, FEBRA2000129, FEBRA2000210, FEBRA2000220, FEBRA2000282, FEBRA2000286, FEBRA2000311, FEBRA2000321, FEBRA2000333, FEBRA2000378, FEBRA2000391, FEBRA2000394, FEBRA2000399, FEBRA2000402, FEBRA2000404, FEBRA2000409, FEBRA2000415, FEBRA2000436, FEBRA2000438, FEBRA2000454, FEBRA2000468, FEBRA2000491, FEBRA2000504, FEBRA2000510, FEBRA2000536, FEBRA2000538, FEBRA2000575, FEBRA2000656, FEBRA2000659, FEBRA2000680, FEBRA2000690, FEBRA2000727, FEBRA2000734, FEBRA2000762, FEBRA2000771, FEBRA2000772, FEBRA2000782,

FEBRA2000790, FEBRA2000803, FEBRA2000805, FEBRA2000809, FEBRA2000856, FEBRA2000862, FEBRA2000877, FEBRA2000880, FEBRA2000901, FEBRA2000909, FEBRA2000917, FEBRA2000928, FEBRA2000936, FEBRA2000937, FEBRA2000959, FEBRA2001004, FEBRA2001012, FEBRA2001020, FEBRA2001093, FEBRA2001124, FEBRA2001133, FEBRA2001161, FEBRA2001175, FEBRA2001217, FEBRA2001267, FEBRA2001294, FEBRA2001334, FEBRA2001351, FEBRA2001383, FEBRA2001438, FEBRA2001440, FEBRA2001487, FEBRA2001523, FEBRA2001561, FEBRA2001584, FEBRA2001590, FEBRA2001591, FEBRA2001610, FEBRA2001620, FEBRA2001659, FEBRA2001698, FEBRA2001705, FEBRA2001706, FEBRA2001752, FEBRA2001772, FEBRA2001790, FEBRA2001914, FEBRA2001974, FEBRA2001989, FEBRA2002009,

FEBRA2002086, FEBRA2002109, FEBRA2002191, FEBRA2002260, FEBRA2002399, FEBRA2002429, FEBRA2002442, FEBRA2002508, FEBRA2002527, FEBRA2002552, FEBRA2002574, FEBRA2002601, FEBRA2002611, FEBRA2002628, FEBRA2002662, FEBRA2002727, FEBRA2002783, FEBRA2002818, FEBRA2002858, FEBRA2002882, FEBRA2002897, FEBRA2002908, FEBRA2002962, FEBRA2002986, FEBRA2003054, FEBRA2003115, FEBRA2003155, FEBRA2003163, FEBRA2003181, FEBRA2003253, FEBRA2003258, FEBRA2003275, FEBRA2003308, FEBRA2003327, FEBRA2003429, FEBRA2003436, FEBRA2003454, FEBRA2003468, FEBRA2003520, FEBRA2003524, FEBRA2003675, FEBRA2003707, FEBRA2003711, FEBRA2003726, FEBRA2003822, FEBRA2003897, FEBRA2003907, FEBRA2004023, FEBRA2004026, FEBRA2004029,

FEBRA2004053, FEBRA2004056, FEBRA2004091, FEBRA2004202, FEBRA2004219, FEBRA2004237, FEBRA2004268, FEBRA2004329, FEBRA2004331, FEBRA2004443, FEBRA2004490, FEBRA2004538, FEBRA2004592, FEBRA2004620, FEBRA2004628, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005216, FEBRA2005291, FEBRA2005377, FEBRA2005380, FEBRA2005427, FEBRA2005458, FEBRA2005701, FEBRA2005726, FEBRA2005752, FEBRA2005787, FEBRA2005788, FEBRA2005998, FEBRA2006055, FEBRA2006061, FEBRA2006150, FEBRA2006165, FEBRA2006315, FEBRA2006346, FEBRA2006354, FEBRA2006370, FEBRA2006379, FEBRA2006409,

FEBRA2006418, FEBRA2006427, FEBRA2006476, FEBRA2006491, FEBRA2006519, FEBRA2006664, FEBRA2006667, FEBRA2006726, FEBRA2006736, FEBRA2006793, FEBRA2006817, FEBRA2006890, FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007534, FEBRA2007551, FEBRA2007566, FEBRA2007589, FEBRA2007613, FEBRA2007620, FEBRA2007708, FEBRA2007818, FEBRA2007823, FEBRA2007931, FEBRA2007957, FEBRA2008009, FEBRA2008086, FEBRA2008134, FEBRA2008135, FEBRA2008159, FEBRA2008165, FEBRA2008172, FEBRA2008255, FEBRA2008265, FEBRA2008266, FEBRA2008282, FEBRA2008311, FEBRA2008341, FEBRA2008459, FEBRA2008468, FEBRA2008475,

FEBRA2008481, FEBRA2008542, FEBRA2008660, FEBRA2008681, FEBRA2008692, FEBRA2008741, FEBRA2008755, FEBRA2008836, FEBRA2008859, FEBRA2008866, FEBRA2008881, FEBRA2008928, FEBRA2008983, FEBRA2009016, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009437, FEBRA2009478, FEBRA2009507, FEBRA2009541, FEBRA2009567, FEBRA2009731, FEBRA2009754, FEBRA2009780, FEBRA2010141, FEBRA2010299, FEBRA2010484, FEBRA2010768, FEBRA2010802, FEBRA2011063, FEBRA2011356, FEBRA2011392, FEBRA2011414, FEBRA2011593, FEBRA2011665, FEBRA2012090,

FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013905, FEBRA2013951, FEBRA2014010, FEBRA2014198, FEBRA2014213, FEBRA2014297, FEBRA2014443, FEBRA2015076, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017098, FEBRA2017138, FEBRA2017154, FEBRA2017200, FEBRA2017223, FEBRA2017333, FEBRA2017502, FEBRA2017533, FEBRA2017680, FEBRA2017736, FEBRA2017780, FEBRA2017811, FEBRA2018051, FEBRA2018433, FEBRA2018746, FEBRA2019172, FEBRA2019298, FEBRA2019389, FEBRA2019457, FEBRA2019519,

FEBRA2019582, FEBRA2019663, FEBRA2019690, FEBRA2019711, FEBRA2019867, FEBRA2020406, FEBRA2020566, FEBRA2020582, FEBRA2020668, FEBRA2020801, FEBRA2020845, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2022013, FEBRA2022148, FEBRA2022204, FEBRA2022255, FEBRA2022322, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023399, FEBRA2023498, FEBRA2023550, FEBRA2023721, FEBRA2023927, FEBRA2023989, FEBRA2023990, FEBRA2024136, FEBRA2024150, FEBRA2024343, FEBRA2024570,

FEBRA2024693, FEBRA2024744, FEBRA2024797, FEBRA2024987, FEBRA2025249, FEBRA2025463, FEBRA2025477, FEBRA2026474, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEHRT2000325, FEHRT2000364, FEKID1000018, FELIV1000101, FELIV1000137, FELIV1000164, FELNG2000241, HCASM1000021, HCASM1000050, HCASM1000061, HCASM1000130, HCASM2000156, HCASM2000202, HCASM2000266, HCASM2000307, HCASM2000363, HCASM2000534, HCASM2000536, HCASM2000560, HCASM2000871, HCASM2001285, HCASM2001810, HCASM2001866, HCASM2001890, HCASM2002148, HCASM2002502,

HCASM2002754, HCASM2002918, HCASM2003076, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000015, HCHON1000142, HCHON1000166, HCHON1000176, HCHON2000038, HCHON2000062, HCHON2000087, HCHON2000100, HCHON2000156, HCHON2000199, HCHON2000271, HCHON2000315, HCHON2000322, HCHON2000344, HCHON2000364, HCHON2000395, HCHON2000417, HCHON2000418, HCHON2000503, HCHON2000626, HCHON2000660, HCHON2000663, HCHON2000673, HCHON2000699, HCHON2000705, HCHON2000817,

HCHON2000818, HCHON2000819, HCHON2000851, HCHON2000898, HCHON2000935, HCHON2000942, HCHON2000956, HCHON2000994, HCHON2001050, HCHON2001099, HCHON2001108, HCHON2001110, HCHON2001116, HCHON2001214, HCHON2001233, HCHON2001269, HCHON2001434, HCHON2001450, HCHON2001453, HCHON2001497, HCHON2001505, HCHON2001519, HCHON2001523, HCHON2001535, HCHON2001546, HCHON2001577, HCHON2001598, HCHON2001604, HCHON2001607, HCHON2001646, HCHON2001712, HCHON2001768, HCHON2001801, HCHON2001853, HCHON2002123, HCHON2002194, HCHON2002241, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659,

HCHON2003676, HCHON2004002, HCHON2004007, HCHON2004279, HCHON2004359, HCHON2004531, HCHON2004776, HCHON2005048, HCHON2005118, HCHON2005166, HCHON2005802, HCHON2005987, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HEART1000010, HEART1000088, HEART1000099, HEART1000102, HEART1000139, HEART1000151, HEART2000024, HEART2000035, HEART2000099, HEART2000298, HEART2000306, HEART2000309, HEART2000418,

HEART2000444, HEART2000448, HEART2000492, HEART2000506, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006557, HEART2006787, HEART2006789, HEART2006909, HEART2007031, HEART2007443, HEART2007767, HEART2008108, HEART2008111, HEART2008257,

HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC2000070, HHDPC2000115, HHDPC2000149, HHDPC2000258, HHDPC2000315, HHDPC2000656, HHDPC2000692, HHDPC2001337, HHDPC2001432, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006862, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HLUNG1000017, HLUNG1000024,

HLUNG1000030, HLUNG1000034, HLUNG1000053, HLUNG1000064, HLUNG1000076, HLUNG1000084, HLUNG1000099, HLUNG1000104, HLUNG1000105, HLUNG1000110, HLUNG1000151, HLUNG1000169, HLUNG2000004, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363, HLUNG2000431, HLUNG2000480, HLUNG2000489, HLUNG2000501, HLUNG2000549, HLUNG2000557, HLUNG2000570, HLUNG2000751, HLUNG2000761, HLUNG2000777, HLUNG2000870, HLUNG2000884, HLUNG2000926, HLUNG2000950, HLUNG2000955, HLUNG2000970, HLUNG2001013, HLUNG2001126,

HLUNG2001144, HLUNG2001214, HLUNG2001439, HLUNG2001459, HLUNG2001507, HLUNG2001814, HLUNG2001996, HLUNG2002006, HLUNG2002050, HLUNG2002059, HLUNG2002085, HLUNG2002145, HLUNG2002295, HLUNG2002303, HLUNG2002405, HLUNG2002429, HLUNG2002465, HLUNG2002562, HLUNG2002648, HLUNG2002659, HLUNG2002707, HLUNG2002811, HLUNG2002916, HLUNG2002942, HLUNG2002949, HLUNG2002958, HLUNG2002982, HLUNG2003003, HLUNG2003049, HLUNG2003061, HLUNG2003162, HLUNG2003246, HLUNG2003331, HLUNG2003335, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004037, HLUNG2004154, HLUNG2004217, HLUNG2004273, HLUNG2004416,

HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006852, HLUNG2006935, HLUNG2007041, HLUNG2007163, HLUNG2007219, HLUNG2007245, HLUNG2007433, HLUNG2007931, HLUNG2008066, HLUNG2008139, HLUNG2008225, HLUNG2008235, HLUNG2008247, HLUNG2008333, HLUNG2008384, HLUNG2008396, HLUNG2008439, HLUNG2008479, HLUNG2008480, HLUNG2008521, HLUNG2008637,

HLUNG2008833, HLUNG2008875, HLUNG2008885, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029,

HLUNG2018282, HLUNG2018626, HLUNG2019058, HSYRA1000131, HSYRA2000044, HSYRA2000237, HSYRA2000253, HSYRA2000371, HSYRA2000424, HSYRA2000507, HSYRA2000510, HSYRA2000629, HSYRA2000640, HSYRA2000706, HSYRA2000760, HSYRA2000787, HSYRA2000792, HSYRA2000832, HSYRA2000927, HSYRA2001003, HSYRA2001103, HSYRA2001142, HSYRA2001153, HSYRA2001232, HSYRA2001255, HSYRA2001332, HSYRA2001353, HSYRA2001420, HSYRA2001484, HSYRA2001552, HSYRA2001580, HSYRA2001631, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007667, HSYRA2008154, HSYRA2008714,

HSYRA2009102, IMR321000155, IMR321000161, IMR321000165, IMR321000193, IMR321000230, IMR321000253, IMR322000010, IMR322000107, IMR322000152, IMR322000164, IMR322000178, IMR322000374, IMR322000379, IMR322000383, IMR322000418, IMR322000518, IMR322000604, IMR322000609, IMR322000656, IMR322000672, IMR322000742, IMR322000811, IMR322000819, IMR322000838, IMR322000859, IMR322000863, IMR322000918, IMR322000919, IMR322000953, IMR322000984, IMR322001018, IMR322001049, IMR322001167, IMR322001185, IMR322001218, IMR322001245, IMR322001318, IMR322001322, IMR322001438, IMR322001452, IMR322001491, IMR322001534, IMR322001584, IMR322001665, IMR322001724, IMR322002035, IMR322002110, IMR322002470, IMR322003675,

IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322013053, IMR322013396, IMR322013731, IMR322013805, IMR322015523, IMR322016146, IMR322017049, IMR322018117, IMR322018192, IMR322019070, JCMLC2000273, KIDNE1000008, KIDNE1000012, KIDNE1000015, KIDNE1000028, KIDNE1000036, KIDNE1000064, KIDNE1000104, KIDNE1000121, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245,

KIDNE2000266, KIDNE2000287, KIDNE2000315, KIDNE2000317, KIDNE2000328, KIDNE2000335, KIDNE2000349, KIDNE2000375, KIDNE2000383, KIDNE2000394, KIDNE2000422, KIDNE2000493, KIDNE2000497, KIDNE2000510, KIDNE2000513, KIDNE2000517, KIDNE2000519, KIDNE2000555, KIDNE2000624, KIDNE2000678, KIDNE2000717, KIDNE2000722, KIDNE2000777, KIDNE2000801, KIDNE2000832, KIDNE2000833, KIDNE2000846, KIDNE2000867, KIDNE2000909, KIDNE2001117, KIDNE2001140, KIDNE2001269, KIDNE2001361, KIDNE2001373, KIDNE2001636, KIDNE2001713, KIDNE2001802, KIDNE2001840, KIDNE2001847, KIDNE2001857, KIDNE2001873, KIDNE2001897, KIDNE2001931, KIDNE2002015, KIDNE2002162, KIDNE2002168, KIDNE2002198, KIDNE2002262, KIDNE2002438, KIDNE2002483,

KIDNE2002725, KIDNE2002768, KIDNE2002795, KIDNE2002798, KIDNE2002839, KIDNE2002845, KIDNE2002872, KIDNE2002882, KIDNE2002980, KIDNE2002991, KIDNE2003185, KIDNE2003188, KIDNE2003233, KIDNE2003316, KIDNE2003335, KIDNE2003357, KIDNE2003373, KIDNE2003377, KIDNE2003507, KIDNE2003752, KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411, KIDNE2004519, KIDNE2004520, KIDNE2004531, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985,

KIDNE2004999, KIDNE2005038, KIDNE2005062, KIDNE2005266, KIDNE2005296, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006580, KIDNE2006652, KIDNE2006687, KIDNE2006733, KIDNE2006760, KIDNE2006820, KIDNE2006880, KIDNE2007005, KIDNE2007040, KIDNE2007077, KIDNE2007186, KIDNE2007222, KIDNE2007356, KIDNE2007422, KIDNE2007569, KIDNE2007810, KIDNE2007811, KIDNE2007910,

KIDNE2007944, KIDNE2008022, KIDNE2008069, KIDNE2008072, KIDNE2008116, KIDNE2008117, KIDNE2008218, KIDNE2008315, KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008975, KIDNE2009109, KIDNE2009180, KIDNE2009191, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010574, KIDNE2010674, KIDNE2010739,

KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012291, KIDNE2012361, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014325, KIDNE2014415,

KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539, KIDNE2016570, KIDNE2016918, KIDNE2017007, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018254, KIDNE2018268, KIDNE2018269,

KIDNE2018287, KIDNE2018294, KIDNE2018352, KIDNE2018462, KIDNE2018493, KIDNE2018617, KIDNE2018678, KIDNE2018727, KIDNE2018863, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000082, LIVER1000099, LIVER1000104, LIVER1000111, LIVER1000126, LIVER1000132, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000037, LIVER2000158, LIVER2000216, LIVER2000237, LIVER2000247, LIVER2000360, LIVER2000416, LIVER2000446, LIVER2000515,

LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001835, LIVER2002638, LIVER2002644, LIVER2002660, LIVER2002702, LIVER2002842, LIVER2003008, LIVER2003065, LIVER2003234, LIVER2003511, LIVER2003524, LIVER2003568, LIVER2003581, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006251, LIVER2006410,

LIVER2006469, LIVER2006644, LIVER2006764, LIVER2007415, LIVER2007548, LIVER2007568, LIVER2007721, LIVER2007783, LIVER2008053, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000096, MAMGL1000100, MAMGL1000132, MAMGL1000151, MAMGL1000178, MAMGL1000182, MAMGL1000184, MESAN1000032, MESAN1000035, MESAN1000079, MESAN1000080, MESAN1000121, MESAN1000180, MESAN2000023, MESAN2000026, MESAN2000067, MESAN2000092, MESAN2000167, MESAN2000264, MESAN2000291, MESAN2000337, MESAN2000434, MESAN2000462, MESAN2000620, MESAN2000711, MESAN2000815, MESAN2000894,

MESAN2000909, MESAN2001154, MESAN2001450, MESAN2001770, MESAN2001979, MESAN2001980, MESAN2002113, MESAN2002147, MESAN2002424, MESAN2002687, MESAN2002709, MESAN2002724, MESAN2002790, MESAN2002978, MESAN2003037, MESAN2003101, MESAN2003190, MESAN2003322, MESAN2003490, MESAN2003529, MESAN2003646, MESAN2003662, MESAN2003709, MESAN2003851, MESAN2004138, MESAN2004288, MESAN2004575, MESAN2005284, MESAN2005303, MESAN2005371, MESAN2005724, MESAN2005808, MESAN2005811, MESAN2005957, MESAN2005958, MESAN2006022, MESAN2006328, MESAN2006580, MESAN2006599, MESAN2006971, MESAN2007032, MESAN2007440, MESAN2007597, MESAN2007755, MESAN2008222, MESAN2008415, MESAN2008460, MESAN2008536, MESAN2008679, MESAN2008729,

MESAN2008821, MESAN2009081, MESAN2009105, MESAN2009311, MESAN2009418, MESAN2009503, MESAN2009522, MESAN2009580, MESAN2010031, MESAN2010114, MESAN2010312, MESAN2010321, MESAN2010664, MESAN2010681, MESAN2011545, MESAN2011597, MESAN2011627, MESAN2011632, MESAN2011977, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013936, MESAN2014192, MESAN2014295, MESAN2014298, MESAN2014412, MESAN2014624, MESAN2015277, MESAN2015365, MESAN2015401, MESAN2015501, MESAN2015708, MESAN2016159, MESAN2016304, MESAN2016409, MESAN2016965, MESAN2017152, MESAN2017373, MESAN2017417, MESAN2018209, MESAN2018576,

MESAN2018670, MESAN2018699, MESTC1000042, MESTC1000147, MESTC2000150, MESTC2000153, MESTC2000170, N1ESE2000698, NB9N41000146, NB9N41000340, NB9N42000104, NB9N42000122, NB9N42000196, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062, NHNPC2000187, NHNPC2000606,

NHNPC2000877, NHNPC2001108, NHNPC2001223, NHNPC2001816, NHNPC2002565, NHNPC2002749, NOVAR1000015, NOVAR1000102, NOVAR2000038, NOVAR2000136, NOVAR2000352, NOVAR2000412, NOVAR2000710, NOVAR2000962, NOVAR2001108, NOVAR2001620, NOVAR2001783, NOVAR2001876, NOVAR2002039, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000068, PEBLM1000071, PEBLM1000083, PEBLM1000147, PEBLM1000174, PEBLM1000180, PEBLM2000030, PEBLM2000112, PEBLM2000126, PEBLM2000170, PEBLM2000180, PEBLM2000213, PEBLM2000248, PEBLM2000267, PEBLM2000270, PEBLM2000321, PEBLM2000333, PEBLM2000338, PEBLM2000479, PEBLM2000502, PEBLM2001312,

PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004497, PEBLM2004666, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006912, PEBLM2007112, PEBLM2007140, PEBLM2007296, PEBLM2007437, PEBLM2007447, PEBLM2007598, PEBLM2007774, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386,

PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005305, PERIC2005347, PERIC2005370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086,

PERIC2009329, PERIC2009566, PLACE5000001, PLACE5000037, PLACE5000058, PLACE5000066, PLACE5000067, PLACE5000068, PLACE5000070, PLACE5000080, PLACE5000105, PLACE5000115, PLACE5000116, PLACE5000129, PLACE5000139, PLACE5000153, PLACE5000170, PLACE5000171, PLACE5000172, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000145, PLACE6000191, PLACE6000205, PLACE6000231, PLACE6000296, PLACE6000314, PLACE6000325, PLACE6000348, PLACE6000371, PLACE6000414, PLACE6000424, PLACE6000426, PLACE6000429, PLACE6000450, PLACE6000463, PLACE6000523, PLACE6000550,

PLACE6000552, PLACE6000555, PLACE6000619, PLACE6000630, PLACE6000953, PLACE6001118, PLACE6001153, PLACE6001185, PLACE6001262, PLACE6001294, PLACE6001536, PLACE6001557, PLACE6001712, PLACE6001740, PLACE6001823, PLACE6001886, PLACE6001925, PLACE6001933, PLACE6002011, PLACE6002016, PLACE6002056, PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6003004, PLACE6003038, PLACE6003077, PLACE6003094, PLACE6003109, PLACE6003204, PLACE6003218, PLACE6003372, PLACE6003399, PLACE6003539, PLACE6003621, PLACE6003705, PLACE6003740, PLACE6003771, PLACE6003796, PLACE6003839,

PLACE6003850, PLACE6004101, PLACE6004219, PLACE6004336, PLACE6004368, PLACE6004380, PLACE6004396, PLACE6004454, PLACE6004464, PLACE6004491, PLACE6004578, PLACE6004663, PLACE6004687, PLACE6004738, PLACE6004966, PLACE6004993, PLACE6005007, PLACE6005029, PLACE6005108, PLACE6005231, PLACE6005234, PLACE6005283, PLACE6005294, PLACE6005328, PLACE6005351, PLACE6005423, PLACE6005487, PLACE6005535, PLACE6005546, PLACE6005559, PLACE6005579, PLACE6005691, PLACE6005786, PLACE6006077, PLACE6006158, PLACE6006186, PLACE6006217, PLACE6006222, PLACE6006266, PLACE6006287, PLACE6006394, PLACE6006418, PLACE6006474, PLACE6006549, PLACE6006566, PLACE6006697, PLACE6006822, PLACE6006871, PLACE6006905, PLACE6007050,

PLACE6007180, PLACE6007239, PLACE6007242, PLACE6007309, PLACE6007373, PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008775, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010077, PLACE6010463, PLACE6010485, PLACE6010568, PLACE6010701, PLACE6010704, PLACE6010732, PLACE6010765, PLACE6010848,

PLACE6010925, PLACE6010936, PLACE6010991, PLACE6011102, PLACE6011156, PLACE6011211, PLACE6011260, PLACE6011774, PLACE6011881, PLACE6011907, PLACE6011975, PLACE6012028, PLACE6012108, PLACE6012168, PLACE6012297, PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016254, PLACE6016383,

PLACE6016942, PLACE6016984, PLACE6017002, PLACE6017431, PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6019195, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004961,

PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000110, PROST1000120, PROST1000123, PROST1000136, PROST1000145, PROST1000152, PROST1000167, PROST1000184, PROST1000217, PROST1000246, PROST1000262, PROST1000272, PROST1000298, PROST1000334, PROST1000343, PROST1000362, PROST1000377, PROST1000383, PROST1000412, PROST1000419, PROST1000451, PROST1000480, PROST1000485, PROST1000493, PROST1000512, PROST1000527,

PROST1000528, PROST1000536, PROST1000559, PROST1000575, PROST2000036, PROST2000053, PROST2000067, PROST2000105, PROST2000143, PROST2000162, PROST2000165, PROST2000176, PROST2000203, PROST2000206, PROST2000212, PROST2000241, PROST2000267, PROST2000273, PROST2000277, PROST2000337, PROST2000432, PROST2000452, PROST2000463, PROST2000470, PROST2000505, PROST2000567, PROST2000572, PROST2000717, PROST2000761, PROST2000835, PROST2000893, PROST2000961, PROST2001002, PROST2001116, PROST2001138, PROST2001283, PROST2001414, PROST2001415, PROST2001465, PROST2001521, PROST2001540, PROST2001599, PROST2001611, PROST2001636, PROST2001739, PROST2001796, PROST2001805, PROST2001823, PROST2001883, PROST2001899,

PROST2001997, PROST2001998, PROST2002078, PROST2002101, PROST2002162, PROST2002212, PROST2002338, PROST2002425, PROST2002488, PROST2002489, PROST2002527, PROST2002591, PROST2002602, PROST2002633, PROST2002682, PROST2002685, PROST2002722, PROST2002736, PROST2002842, PROST2002897, PROST2002906, PROST2002927, PROST2003102, PROST2003117, PROST2003210, PROST2003302, PROST2003303, PROST2003324, PROST2003340, PROST2003428, PROST2003472, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251,

PROST2004332, PROST2004481, PROST2004526, PROST2004727, PROST2004742, PROST2004744, PROST2004817, PROST2005039, PROST2005067, PROST2005076, PROST2005093, PROST2005121, PROST2005131, PROST2005228, PROST2005285, PROST2005426, PROST2005466, PROST2005604, PROST2005630, PROST2005683, PROST2005788, PROST2005880, PROST2005886, PROST2005904, PROST2005919, PROST2005943, PROST2006008, PROST2006020, PROST2006060, PROST2006196, PROST2006201, PROST2006282, PROST2006450, PROST2006491, PROST2006510, PROST2006579, PROST2006688, PROST2006737, PROST2006766, PROST2006782, PROST2006933, PROST2006988, PROST2007122, PROST2007237, PROST2007248, PROST2007289, PROST2007317, PROST2007328, PROST2007382, PROST2007389,

PROST2007416, PROST2007528, PROST2007612, PROST2007818, PROST2007950, PROST2007956, PROST2007974, PROST2007988, PROST2008079, PROST2008088, PROST2008243, PROST2008245, PROST2008268, PROST2008360, PROST2008447, PROST2008468, PROST2008472, PROST2008516, PROST2008724, PROST2008835, PROST2008993, PROST2009022, PROST2009208, PROST2009222, PROST2009254, PROST2009273, PROST2009320, PROST2009347, PROST2009365, PROST2009388, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010579, PROST2010606, PROST2010885, PROST2011012,

PROST2011038, PROST2011105, PROST2011297, PROST2011410, PROST2011439, PROST2011482, PROST2011631, PROST2011660, PROST2012005, PROST2012016, PROST2012088, PROST2012140, PROST2012157, PROST2012190, PROST2012249, PROST2012353, PROST2012400, PROST2012504, PROST2012542, PROST2012550, PROST2012740, PROST2012745, PROST2012780, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015198, PROST2015243, PROST2015246, PROST2015251, PROST2015287, PROST2015457, PROST2015464, PROST2015537, PROST2015545,

PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016980, PROST2017098, PROST2017128, PROST2017367, PROST2017413, PROST2017441, PROST2017529, PROST2017578, PROST2017612, PROST2017617, PROST2017700, PROST2017701, PROST2017729, PROST2017836, PROST2017972, PROST2018030, PROST2018090, PROST2018235, PROST2018437, PROST2018487, PROST2018511, PROST2018583, PROST2018588, PROST2018788, PROST2018902, PROST2018922, PROST2018977,

PROST2019164, PROST2019296, PROST2019398, PROST2019487, PROST2019781, PUAEN1000039, PUAEN1000081, PUAEN1000085, PUAEN1000087, PUAEN1000121, PUAEN1000147, PUAEN1000175, PUAEN1000275, PUAEN1000276, PUAEN1000288, PUAEN2000080, PUAEN2000152, PUAEN2000175, PUAEN2000247, PUAEN2000312, PUAEN2000394, PUAEN2000535, PUAEN2000594, PUAEN2000684, PUAEN2000942, PUAEN2001260, PUAEN2001345, PUAEN2001526, PUAEN2001586, PUAEN2001882, PUAEN2002473, PUAEN2002552, PUAEN2002568, PUAEN2002616, PUAEN2002758, PUAEN2003018, PUAEN2003079, PUAEN2003116, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588,

PUAEN2005930, PUAEN2006029, PUAEN2006328, PUAEN2006578, PUAEN2006639, PUAEN2006761, PUAEN2007785, PUAEN2007898, PUAEN2008123, PUAEN2008228, PUAEN2008314, PUAEN2008515, PUAEN2008939, PUAEN2008980, PUAEN2008985, PUAEN2009174, PUAEN2009348, PUAEN2009534, PUAEN2009655, PUAEN2009795, PUAEN2009852, PUAEN2010346, PUAEN2010824, RECTM1000141, RECTM2000220, RECTM2000349, RECTM2000433, RECTM2001307, RECTM2001519, RECTM2001666, RECTM2001691, RECTM2001749, RECTM2002172, RECTM2002602, RECTM2002632, SALGL1000005, SALGL1000024, SALGL1000028, SALGL1000065, SALGL1000107, SALGL1000171, SKMUS1000014, SKMUS1000030, SKMUS1000067, SKMUS1000102, SKMUS1000104, SKMUS1000110, SKMUS1000118, SKMUS1000124, SKMUS1000129,

SKMUS1000176, SKMUS1000177, SKMUS2000020, SKMUS2000061, SKMUS2000074, SKMUS2000078, SKMUS2000117, SKMUS2000137, SKMUS2000243, SKMUS2000271, SKMUS2000287, SKMUS2000317, SKMUS2000330, SKMUS2000339, SKMUS2000343, SKMUS2000365, SKMUS2000380, SKMUS2000390, SKMUS2000416, SKMUS2000458, SKMUS2000468, SKMUS2000484, SKMUS2000515, SKMUS2000690, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000873, SKMUS2000894, SKMUS2000898, SKMUS2000902, SKMUS2000933, SKMUS2000945, SKMUS2000954, SKMUS2000961, SKMUS2000973, SKMUS2000980, SKMUS2001008, SKMUS2001014, SKMUS2001129, SKMUS2001157, SKMUS2001164,

SKMUS2001199, SKMUS2001201, SKMUS2001215, SKMUS2001247, SKMUS2001254, SKMUS2001271, SKMUS2001287, SKMUS2001364, SKMUS2001398, SKMUS2001402, SKMUS2001412, SKMUS2001492, SKMUS2001501, SKMUS2001528, SKMUS2001543, SKMUS2001580, SKMUS2001585, SKMUS2001608, SKMUS2001631, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897,

SKMUS2004903, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000123, SKNMC1000124, SKNMC1000137, SKNMC1000159, SKNMC1000251, SKNMC2000224, SKNMC2000256, SKNMC2000305, SKNMC2000322, SKNMC2000356, SKNMC2000374, SKNMC2000583, SKNMC2000593, SKNMC2000612, SKNMC2000622, SKNMC2000966, SKNMC2001113, SKNMC2001503, SKNMC2001555, SKNMC2001596, SKNMC2001623, SKNMC2003639, SKNMC2003924, SKNMC2003987, SKNMC2004457, SKNMC2004643, SKNMC2004651, SKNMC2005772, SKNMC2006327, SKNMC2006998,

SKNMC2007134, SKNMC2007502, SKNMC2007504, SKNMC2007961, SKNMC2009450, SKNSH1000174, SKNSH1000301, SKNSH1000308, SKNSH1000416, SKNSH2000151, SKNSH2000163, SKNSH2000319, SKNSH2000482, SKNSH2000516, SKNSH2000550, SKNSH2000716, SKNSH2000971, SKNSH2001222, SKNSH2001875, SKNSH2002054, SKNSH2002768, SKNSH2002866, SKNSH2003064, SKNSH2003174, SKNSH2003422, SKNSH2003466, SKNSH2003483, SKNSH2003528, SKNSH2003633, SKNSH2004039, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008940, SKNSH2008969, SKNSH2009197, SKNSH2009357, SKNSH2009435, SKNSH2009991,

SKNSH2010015, SMINT1000012, SMINT1000016, SMINT1000039, SMINT1000048, SMINT1000051, SMINT1000057, SMINT1000071, SMINT1000075, SMINT1000078, SMINT1000091, SMINT1000100, SMINT1000103, SMINT1000118, SMINT1000137, SMINT1000192, SMINT2000007, SMINT2000032, SMINT2000040, SMINT2000060, SMINT2000084, SMINT2000145, SMINT2000159, SMINT2000185, SMINT2000227, SMINT2000229, SMINT2000239, SMINT2000267, SMINT2000454, SMINT2000518, SMINT2000529, SMINT2000530, SMINT2000538, SMINT2000541, SMINT2000545, SMINT2000558, SMINT2000562, SMINT2000568, SMINT2000602, SMINT2000629, SMINT2000747, SMINT2000811, SMINT2000824, SMINT2000933, SMINT2000984, SMINT2001114, SMINT2001158, SMINT2001183, SMINT2001199, SMINT2001222,

SMINT2001348, SMINT2001397, SMINT2001458, SMINT2001461, SMINT2001535, SMINT2001559, SMINT2001609, SMINT2001683, SMINT2001731, SMINT2001812, SMINT2001818, SMINT2001950, SMINT2002126, SMINT2002159, SMINT2002202, SMINT2002210, SMINT2002281, SMINT2002311, SMINT2002314, SMINT2002328, SMINT2002414, SMINT2002457, SMINT2002689, SMINT2002743, SMINT2002778, SMINT2002880, SMINT2002884, SMINT2002899, SMINT2002974, SMINT2003003, SMINT2003128, SMINT2003150, SMINT2003317, SMINT2003340, SMINT2003386, SMINT2003423, SMINT2003505, SMINT2003569, SMINT2003641, SMINT2003644, SMINT2003866, SMINT2004023, SMINT2004037, SMINT2004047, SMINT2004086, SMINT2004144, SMINT2004280, SMINT2004299, SMINT2004339, SMINT2004350,

SMINT2004414, SMINT2004473, SMINT2004547, SMINT2004583, SMINT2004589, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005387, SMINT2005402, SMINT2005405, SMINT2005621, SMINT2005623, SMINT2005624, SMINT2005688, SMINT2005800, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796, SMINT2007867,

SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008625, SMINT2008690, SMINT2008715, SMINT2008720, SMINT2008817, SMINT2008844, SMINT2008869, SMINT2008917, SMINT2009119, SMINT2009121, SMINT2009212, SMINT2009216, SMINT2009233, SMINT2009259, SMINT2009272, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010500, SMINT2010533, SMINT2010629, SMINT2010672, SMINT2010753,

SMINT2010853, SMINT2010897, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012122, SMINT2012179, SMINT2012195, SMINT2012285, SMINT2012501, SMINT2012734, SMINT2012793, SMINT2013031, SMINT2013032, SMINT2013129, SMINT2013170, SMINT2013181, SMINT2013228, SMINT2013613, SMINT2013626, SMINT2013695, SMINT2013833, SMINT2014166, SMINT2014282, SMINT2014443, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146,

SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000134, SPLEN1000139, SPLEN1000143, SPLEN1000144, SPLEN1000145, SPLEN1000156, SPLEN1000163, SPLEN1000178, SPLEN2000020, SPLEN2000047, SPLEN2000064,

SPLEN2000072, SPLEN2000098, SPLEN2000126, SPLEN2000134, SPLEN2000143, SPLEN2000189, SPLEN2000197, SPLEN2000205, SPLEN2000210, SPLEN2000222, SPLEN2000242, SPLEN2000243, SPLEN2000247, SPLEN2000255, SPLEN2000270, SPLEN2000307, SPLEN2000310, SPLEN2000341, SPLEN2000348, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000537, SPLEN2000541, SPLEN2000607, SPLEN2000630, SPLEN2000695, SPLEN2000874, SPLEN2001135, SPLEN2001141, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599,

SPLEN2001626, SPLEN2001650, SPLEN2001689, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002343, SPLEN2002354, SPLEN2002385, SPLEN2002419, SPLEN2002451, SPLEN2002462, SPLEN2002463, SPLEN2002477, SPLEN2002662, SPLEN2002695, SPLEN2002707, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004124, SPLEN2004148, SPLEN2004181, SPLEN2004220, SPLEN2004343, SPLEN2004346, SPLEN2004368, SPLEN2004611,

SPLEN2004662, SPLEN2004844, SPLEN2004880, SPLEN2004984, SPLEN2005227, SPLEN2005272, SPLEN2005342, SPLEN2005416, SPLEN2005429, SPLEN2005450, SPLEN2005474, SPLEN2005560, SPLEN2005727, SPLEN2005790, SPLEN2005806, SPLEN2005818, SPLEN2005838, SPLEN2005869, SPLEN2005927, SPLEN2005939, SPLEN2006232, SPLEN2006268, SPLEN2006283, SPLEN2006389, SPLEN2006425, SPLEN2006671, SPLEN2006701, SPLEN2006785, SPLEN2006875, SPLEN2007276, SPLEN2007314, SPLEN2007326, SPLEN2007350, SPLEN2007388, SPLEN2007498, SPLEN2007619, SPLEN2007653, SPLEN2007689, SPLEN2007695, SPLEN2007750, SPLEN2007794, SPLEN2007879, SPLEN2007926, SPLEN2007951, SPLEN2008007, SPLEN2008135, SPLEN2008164, SPLEN2008317, SPLEN2008496, SPLEN2008591,

SPLEN2008786, SPLEN2009081, SPLEN2009088, SPLEN2009163, SPLEN2009315, SPLEN2009340, SPLEN2009512, SPLEN2009513, SPLEN2009541, SPLEN2009548, SPLEN2009581, SPLEN2009733, SPLEN2009803, SPLEN2009884, SPLEN2009918, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011419, SPLEN2011422, SPLEN2011672, SPLEN2011678, SPLEN2011766,

SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012523, SPLEN2012524, SPLEN2012525, SPLEN2012606, SPLEN2012611, SPLEN2012619, SPLEN2012624, SPLEN2012725, SPLEN2012743, SPLEN2012756, SPLEN2012800, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014119, SPLEN2014136, SPLEN2014199, SPLEN2014210, SPLEN2014293, SPLEN2014318,

SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860, SPLEN2014865, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016304,

SPLEN2016356, SPLEN2016421, SPLEN2016480, SPLEN2016531, SPLEN2016533, SPLEN2016541, SPLEN2016552, SPLEN2016554, SPLEN2016602, SPLEN2016627, SPLEN2016678, SPLEN2016743, SPLEN2016773, SPLEN2016781, SPLEN2016840, SPLEN2016863, SPLEN2016922, SPLEN2016932, SPLEN2016973, SPLEN2017043, SPLEN2017104, SPLEN2017121, SPLEN2017147, SPLEN2017187, SPLEN2017189, SPLEN2017212, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017918, SPLEN2017999, SPLEN2018098, SPLEN2018112, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683,

SPLEN2018732, SPLEN2018749, SPLEN2018933, SPLEN2019008, SPLEN2019043, SPLEN2019077, SPLEN2019092, SPLEN2019102, SPLEN2019131, SPLEN2019169, SPLEN2019257, SPLEN2019311, SPLEN2019323, SPLEN2019349, SPLEN2019375, SPLEN2019379, SPLEN2019405, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021157, SPLEN2021231, SPLEN2021273, SPLEN2021295, SPLEN2021298,

SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025039, SPLEN2025219, SPLEN2025491, SPLEN2025626, SPLEN2025820, SPLEN2026144, SPLEN2026411, SPLEN2026765, SPLEN2027157, SPLEN2027395,

SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333,

SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039672, SPLEN2039697, SPLEN2039845, SPLEN2039936, SPLEN2040222, SPLEN2040237,

SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042521, SPLEN2042535, SPLEN2042598, SPLEN2042714, SPLEN2042920, STOMA1000013, STOMA1000052, STOMA1000074, STOMA1000091, STOMA1000117, STOMA1000133, STOMA1000175, STOMA1000186, STOMA1000189, STOMA1000190, STOMA1000191, STOMA2000032, STOMA2000072, STOMA2000088, STOMA2000104, STOMA2000148, STOMA2000168, STOMA2000183, STOMA2000188, STOMA2000257, STOMA2000279, STOMA2000383, STOMA2000386, STOMA2000395, STOMA2000478, STOMA2000529, STOMA2000539, STOMA2000567, STOMA2000640, STOMA2000678, STOMA2000686, STOMA2000888, STOMA2001025, STOMA2001389, STOMA2002052,

STOMA2002141, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000128, SYNOV1000164,

SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001212, SYNOV2001239, SYNOV2001251, SYNOV2001262, SYNOV2001300, SYNOV2001356, SYNOV2001390, SYNOV2001451, SYNOV2001457, SYNOV2001468, SYNOV2001581, SYNOV2001648, SYNOV2001660, SYNOV2003326, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2009172, SYNOV2011233,

SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001153, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012,

SYNOV4007215, SYNOV4007360, SYNOV4007430, SYNOV4007521, SYNOV4007553, SYNOV4007711, SYNOV4008245, SYNOV4008336, SYNOV4009139, SYNOV4009575, T1ESE2000116, T1ESE2000609, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133,

TBAES2009387, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002489, TESOP2002539, TESOP2002545, TESOP2002804, TESOP2002950, TESOP2003273, TESOP2003308, TESOP2003553, TESOP2003753, TESOP2004110,

TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000055, TESTI1000064, TESTI1000070, TESTI1000085, TESTI1000088, TESTI1000093,

TESTI1000094, TESTI1000101, TESTI1000109, TESTI1000125, TESTI1000127, TESTI1000128, TESTI1000131, TESTI1000138, TESTI1000157, TESTI1000163, TESTI1000168, TESTI1000169, TESTI1000179, TESTI1000183, TESTI1000191, TESTI1000257, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000177, TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462,

TESTI2000489, TESTI2000520, TESTI2000571, TESTI2000591, TESTI2000600, TESTI2000616, TESTI2000644, TESTI2000671, TESTI2000683, TESTI2000689, TESTI2000695, TESTI2000699, TESTI2000707, TESTI2000745, TESTI2000756, TESTI2000762, TESTI2000808, TESTI2000819, TESTI2000830, TESTI2000840, TESTI2000849, TESTI2000850, TESTI2000883, TESTI2000909, TESTI2000951, TESTI2000981, TESTI2001008, TESTI2001049, TESTI2001082, TESTI2001120, TESTI2001134, TESTI2001141, TESTI2001153, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001512, TESTI2001518,

TESTI2001556, TESTI2001593, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001829, TESTI2001852, TESTI2001862, TESTI2001879, TESTI2001894, TESTI2001898, TESTI2001915, TESTI2001950, TESTI2001959, TESTI2001968, TESTI2001991, TESTI2002002, TESTI2002012, TESTI2002036, TESTI2002048, TESTI2002057, TESTI2002081, TESTI2002090, TESTI2002105, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002406,

TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002724, TESTI2002729, TESTI2002752, TESTI2002753, TESTI2002789, TESTI2002802, TESTI2002806, TESTI2002840, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003130, TESTI2003131, TESTI2003139, TESTI2003141,

TESTI2003152, TESTI2003181, TESTI2003193, TESTI2003196, TESTI2003228, TESTI2003236, TESTI2003255, TESTI2003258, TESTI2003277, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003418, TESTI2003419, TESTI2003432, TESTI2003454, TESTI2003475, TESTI2003476, TESTI2003498, TESTI2003512, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824,

TESTI2003827, TESTI2003894, TESTI2003911, TESTI2003914, TESTI2003940, TESTI2003962, TESTI2003998, TESTI2004000, TESTI2004073, TESTI2004080, TESTI2004085, TESTI2004089, TESTI2004111, TESTI2004119, TESTI2004163, TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004783, TESTI2004791,

TESTI2004793, TESTI2004860, TESTI2004906, TESTI2004920, TESTI2004936, TESTI2004941, TESTI2004971, TESTI2004982, TESTI2004993, TESTI2004994, TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720,

TESTI2005739, TESTI2005759, TESTI2005767, TESTI2005775, TESTI2005788, TESTI2005827, TESTI2005835, TESTI2005860, TESTI2005892, TESTI2005908, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006051, TESTI2006067, TESTI2006083, TESTI2006097, TESTI2006109, TESTI2006111, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006409, TESTI2006425, TESTI2006437,

TESTI2006453, TESTI2006457, TESTI2006460, TESTI2006465, TESTI2006483, TESTI2006499, TESTI2006523, TESTI2006543, TESTI2006565, TESTI2006572, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006774, TESTI2006853, TESTI2006866, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007220, TESTI2007234, TESTI2007311, TESTI2007320, TESTI2007346, TESTI2007358, TESTI2007402, TESTI2007405,

TESTI2007407, TESTI2007422, TESTI2007452, TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405,

TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786, TESTI2008822, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009390, TESTI2009400, TESTI2009412, TESTI2009423, TESTI2009447, TESTI2009462, TESTI2009477, TESTI2009502, TESTI2009511, TESTI2009544, TESTI2009545, TESTI2009551, TESTI2009585, TESTI2009588, TESTI2009683, TESTI2009727, TESTI2009739, TESTI2009812, TESTI2009835,

TESTI2009853, TESTI2009935, TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010682, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011712, TESTI2011750, TESTI2011840, TESTI2011846, TESTI2011915, TESTI2011928, TESTI2011971, TESTI2012019,

TESTI2012050, TESTI2012090, TESTI2012095, TESTI2012104, TESTI2012152, TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012778, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257, TESTI2013264, TESTI2013268, TESTI2013322, TESTI2013381, TESTI2013382, TESTI2013401, TESTI2013427, TESTI2013468, TESTI2013497, TESTI2013545,

TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988, TESTI2015042, TESTI2015092, TESTI2015105, TESTI2015180, TESTI2015249, TESTI2015327, TESTI2015331, TESTI2015335, TESTI2015350, TESTI2015375, TESTI2015437, TESTI2015590,

TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256,

TESTI2017305, TESTI2017311, TESTI2017396, TESTI2017507, TESTI2017537, TESTI2017582, TESTI2017584, TESTI2017727, TESTI2017740, TESTI2017775, TESTI2017816, TESTI2017832, TESTI2017923, TESTI2017932, TESTI2017951, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018941, TESTI2018957, TESTI2018974, TESTI2018975, TESTI2018997, TESTI2019042, TESTI2019077, TESTI2019228, TESTI2019257, TESTI2019280, TESTI2019282, TESTI2019308, TESTI2019336,

TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020515, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112,

TESTI2021114, TESTI2021116, TESTI2021118, TESTI2021122, TESTI2021138, TESTI2021297, TESTI2021315, TESTI2021358, TESTI2021425, TESTI2021463, TESTI2021531, TESTI2021599, TESTI2021637, TESTI2021654, TESTI2021667, TESTI2021736, TESTI2021785, TESTI2021819, TESTI2021895, TESTI2021911, TESTI2021939, TESTI2021999, TESTI2022010, TESTI2022023, TESTI2022065, TESTI2022086, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214,

TESTI2023254, TESTI2023386, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174,

TESTI2025229, TESTI2025269, TESTI2025365, TESTI2025403, TESTI2025409, TESTI2025422, TESTI2025448, TESTI2025454, TESTI2025486, TESTI2025546, TESTI2025582, TESTI2025609, TESTI2025656, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026534, TESTI2026537, TESTI2026589, TESTI2026597, TESTI2026605, TESTI2026647, TESTI2026674, TESTI2026794, TESTI2026824, TESTI2026854,

TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776,

TESTI2028811, TESTI2028891, TESTI2028985, TESTI2028997, TESTI2029131, TESTI2029162, TESTI2029196, TESTI2029201, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681,

TESTI2032761, TESTI2032768, TESTI2032774, TESTI2032828, TESTI2033031, TESTI2033242, TESTI2033254, TESTI2033300, TESTI2033395, TESTI2033441, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688,

TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275,

TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664,

TESTI2041730, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155,

TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206,

TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI3000002, TESTI3000005, TESTI4000068, TESTI4000079, TESTI4000093, TESTI4000134,

TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001467, TESTI4001473, TESTI4001517, TESTI4001527, TESTI4001561, TESTI4001569, TESTI4001604, TESTI4001665, TESTI4001679, TESTI4001795, TESTI4001923, TESTI4001925, TESTI4001984, TESTI4002003, TESTI4002141,

TESTI4002195, TESTI4002202, TESTI4002290, TESTI4002319, TESTI4002491, TESTI4002520, TESTI4002552, TESTI4002703, TESTI4002754, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005158, TESTI4005317, TESTI4005322, TESTI4005470, TESTI4005500, TESTI4005534, TESTI4005543, TESTI4005628, TESTI4005801, TESTI4005805, TESTI4005857,

TESTI4005961, TESTI4006053, TESTI4006079, TESTI4006112, TESTI4006137, TESTI4006148, TESTI4006219, TESTI4006234, TESTI4006308, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4008007, TESTI4008018, TESTI4008050, TESTI4008086, TESTI4008097, TESTI4008176, TESTI4008209, TESTI4008219, TESTI4008250, TESTI4008302, TESTI4008305,

TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505,

TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4013365, TESTI4013369, TESTI4013410, TESTI4013474, TESTI4013602, TESTI4013667, TESTI4013675, TESTI4013685, TESTI4013735, TESTI4013742, TESTI4013774, TESTI4013817, TESTI4013830, TESTI4013852, TESTI4013894, TESTI4013924, TESTI4013960, TESTI4013962, TESTI4014159, TESTI4014265, TESTI4014276, TESTI4014306, TESTI4014392, TESTI4014415, TESTI4014445, TESTI4014553, TESTI4014661,

TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017763, TESTI4017848, TESTI4017854, TESTI4017901, TESTI4017961, TESTI4017984, TESTI4018101,

TESTI4018152, TESTI4018208, TESTI4018382, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096,

TESTI4024240, TESTI4024245, TESTI4024294, TESTI4024344, TESTI4024387, TESTI4024420, TESTI4024494, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809,

TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065,

TESTI4035498, TESTI4035508, TESTI4035581, TESTI4035602, TESTI4035637, TESTI4035649, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939,

TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168, TESTI4045312, TESTI4045330, TESTI4045470, TESTI4045701,

TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000017, THYMU1000025, THYMU1000029, THYMU1000032, THYMU1000033, THYMU1000060, THYMU1000074, THYMU1000098, THYMU1000105, THYMU1000109, THYMU1000136, THYMU1000142, THYMU1000158, THYMU1000176, THYMU1000179, THYMU1000191, THYMU1000198, THYMU1000217, THYMU1000240, THYMU1000242,

THYMU1000291, THYMU1000316, THYMU1000318, THYMU1000331, THYMU1000336, THYMU1000359, THYMU1000366, THYMU1000382, THYMU1000393, THYMU1000394, THYMU1000399, THYMU1000426, THYMU1000440, THYMU1000459, THYMU1000473, THYMU1000490, THYMU1000491, THYMU1000496, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000382, THYMU2000418, THYMU2000440, THYMU2000489, THYMU2000492,

THYMU2000519, THYMU2000570, THYMU2000602, THYMU2000660, THYMU2000671, THYMU2000672, THYMU2000684, THYMU2000767, THYMU2000775, THYMU2000800, THYMU2000879, THYMU2000932, THYMU2000950, THYMU2000971, THYMU2000982, THYMU2000990, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001422, THYMU2001443, THYMU2001515, THYMU2001521, THYMU2001556, THYMU2001622, THYMU2001727, THYMU2001819, THYMU2001825, THYMU2001839, THYMU2001868, THYMU2001898, THYMU2001900, THYMU2001916, THYMU2001989, THYMU2002023, THYMU2002037, THYMU2002080, THYMU2002109,

THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002815, THYMU2002852, THYMU2002910, THYMU2002950, THYMU2002983, THYMU2003012, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003470, THYMU2003479, THYMU2003499, THYMU2003542, THYMU2003632, THYMU2003650, THYMU2003700, THYMU2003760, THYMU2003803,

THYMU2003855, THYMU2003891, THYMU2003907, THYMU2003932, THYMU2004003, THYMU2004008, THYMU2004042, THYMU2004108, THYMU2004111, THYMU2004123, THYMU2004139, THYMU2004169, THYMU2004284, THYMU2004320, THYMU2004336, THYMU2004356, THYMU2004410, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507,

THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006913, THYMU2006946, THYMU2006965, THYMU2007025, THYMU2007060, THYMU2007095, THYMU2007146, THYMU2007179, THYMU2007258, THYMU2007307, THYMU2007308, THYMU2007350, THYMU2007384, THYMU2007415, THYMU2007467, THYMU2007493, THYMU2007521, THYMU2007528, THYMU2007530, THYMU2007532,

THYMU2007561, THYMU2007571, THYMU2007635, THYMU2007658, THYMU2007667, THYMU2007725, THYMU2007729, THYMU2007802, THYMU2007824, THYMU2007854, THYMU2007952, THYMU2007969, THYMU2008035, THYMU2008036, THYMU2008072, THYMU2008111, THYMU2008145, THYMU2008149, THYMU2008185, THYMU2008191, THYMU2008207, THYMU2008226, THYMU2008282, THYMU2008339, THYMU2008350, THYMU2008351, THYMU2008383, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186,

THYMU2009338, THYMU2009425, THYMU2009516, THYMU2009526, THYMU2009546, THYMU2009596, THYMU2009658, THYMU2009792, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011538, THYMU2011548, THYMU2011551, THYMU2011564, THYMU2011573, THYMU2011585, THYMU2011621, THYMU2011666,

THYMU2011736, THYMU2011785, THYMU2011806, THYMU2011852, THYMU2011937, THYMU2011939, THYMU2012073, THYMU2012104, THYMU2012113, THYMU2012273, THYMU2012401, THYMU2012492, THYMU2012507, THYMU2012565, THYMU2012574, THYMU2012631, THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014183, THYMU2014204, THYMU2014297, THYMU2014323, THYMU2014327, THYMU2014353, THYMU2014492,

THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015775, THYMU2015825, THYMU2015918, THYMU2015943, THYMU2015984, THYMU2016113, THYMU2016164, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071,

THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943,

THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2026182, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068,

THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032976, THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338,

THYMU2034374, THYMU2034647, THYMU2035064, THYMU2035078, THYMU2035101, THYMU2035319, THYMU2035321, THYMU2035388, THYMU2035400, THYMU2035554, THYMU2035710, THYMU2035735, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634,

THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001991, THYMU3002452, THYMU3002661, THYMU3003007, THYMU3003092, THYMU3003212, THYMU3003350, THYMU3003403,

THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012,

THYMU3011081, THYMU3011244, THYMU3011360, THYMU3011534, THYMU3011543, THYMU3011556, THYMU3011717, THYMU3012402, THYMU3012907, THYMU3012983, THYMU3013114, THYMU3013197, THYMU3013241, THYMU3013386, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869,

THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752,

THYMU3031146, THYMU3031402, THYMU3031610, THYMU3031612, THYMU3031868, THYMU3031878, THYMU3032032, THYMU3032867, THYMU3033402, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126,

THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047144, THYMU3047156, THYMU3047366, THYMU3047513, THYMU3047542, THYMU3047760, THYMU3047891, TKIDN1000001, TKIDN1000062, TKIDN1000066,

TKIDN1000164, TKIDN1000171, TKIDN2000240, TKIDN2000319, TKIDN2000464, TKIDN2000521, TKIDN2000701, TKIDN2001662, TKIDN2002318, TKIDN2002329, TKIDN2002424, TKIDN2002632, TKIDN2003044, TKIDN2003059, TKIDN2003062, TKIDN2003366, TKIDN2003396, TKIDN2003413, TKIDN2003597, TKIDN2003601, TKIDN2004185, TKIDN2004386, TKIDN2004458, TKIDN2004748, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009794, TKIDN2009889, TKIDN2010200, TKIDN2010232, TKIDN2010278, TKIDN2010602, TKIDN2011051, TKIDN2011160, TKIDN2011324, TKIDN2012034, TKIDN2012771,

TKIDN2012824, TKIDN2013134, TKIDN2014570, TKIDN2014757, TKIDN2014771, TKIDN2015025, TKIDN2015071, TKIDN2015161, TKIDN2015263, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002336, TLIVE2002338, TLIVE2002562, TLIVE2002882, TLIVE2003197, TLIVE2003200, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733,

TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000038, TRACH1000057, TRACH1000063, TRACH1000074, TRACH1000100, TRACH1000125, TRACH1000140, TRACH1000144, TRACH1000193, TRACH1000212, TRACH1000240, TRACH2000002, TRACH2000015, TRACH2000024, TRACH2000032, TRACH2000045, TRACH2000047,

TRACH2000081, TRACH2000083, TRACH2000148, TRACH2000185, TRACH2000196, TRACH2000235, TRACH2000243, TRACH2000250, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000496, TRACH2000497, TRACH2000502, TRACH2000522, TRACH2000540, TRACH2000559, TRACH2000660, TRACH2000665, TRACH2000675, TRACH2000677, TRACH2000703, TRACH2000767, TRACH2000775, TRACH2000807, TRACH2000862, TRACH2000873, TRACH2000894, TRACH2000898, TRACH2000923, TRACH2000944, TRACH2000972, TRACH2000981, TRACH2000986, TRACH2001037, TRACH2001060, TRACH2001085, TRACH2001101, TRACH2001121, TRACH2001147, TRACH2001154, TRACH2001188,

TRACH2001192, TRACH2001249, TRACH2001275, TRACH2001335, TRACH2001339, TRACH2001341, TRACH2001347, TRACH2001395, TRACH2001400, TRACH2001403, TRACH2001416, TRACH2001428, TRACH2001432, TRACH2001443, TRACH2001474, TRACH2001523, TRACH2001525, TRACH2001532, TRACH2001549, TRACH2001592, TRACH2001596, TRACH2001621, TRACH2001646, TRACH2001651, TRACH2001684, TRACH2001933, TRACH2001968, TRACH2001996, TRACH2002043, TRACH2002054, TRACH2002092, TRACH2002100, TRACH2002138, TRACH2002333, TRACH2002664, TRACH2002784, TRACH2002840, TRACH2002847, TRACH2002853, TRACH2002862, TRACH2002954, TRACH2002960, TRACH2002988, TRACH2003070, TRACH2003484, TRACH2003516, TRACH2003605, TRACH2003736, TRACH2003759, TRACH2003941,

TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007483, TRACH2007542, TRACH2007577,

TRACH2007674, TRACH2007676, TRACH2007688, TRACH2007733, TRACH2007754, TRACH2007816, TRACH2007894, TRACH2007969, TRACH2007988, TRACH2008112, TRACH2008113, TRACH2008127, TRACH2008278, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639,

TRACH2010677, TRACH2010698, TRACH2010824, TRACH2010883, TRACH2010965, TRACH2010974, TRACH2011057, TRACH2011093, TRACH2011113, TRACH2011293, TRACH2011302, TRACH2011500, TRACH2011574, TRACH2011956, TRACH2012000, TRACH2012138, TRACH2012242, TRACH2012370, TRACH2012376, TRACH2012387, TRACH2012497, TRACH2012536, TRACH2012562, TRACH2012742, TRACH2012811, TRACH2012823, TRACH2012918, TRACH2013081, TRACH2013265, TRACH2013369, TRACH2013450, TRACH2013495, TRACH2013585, TRACH2013671, TRACH2013726, TRACH2013902, TRACH2013928, TRACH2013982, TRACH2013991, TRACH2014018, TRACH2014045, TRACH2014046, TRACH2014077, TRACH2014082, TRACH2014268, TRACH2014284, TRACH2014337, TRACH2014356, TRACH2014371, TRACH2014442,

TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084,

TRACH2018262, TRACH2018273, TRACH2018278, TRACH2018317, TRACH2018345, TRACH2018380, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479,

TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000180, TRACH3000342, TRACH3000420, TRACH3000515, TRACH3000530, TRACH3000548, TRACH3000558, TRACH3000586, TRACH3000780, TRACH3000892, TRACH3000926, TRACH3001119, TRACH3001443, TRACH3001550, TRACH3001759, TRACH3002064,

TRACH3002107, TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003586, TRACH3003683, TRACH3003780, TRACH3003805, TRACH3003832, TRACH3004068, TRACH3004113, TRACH3004288, TRACH3004303, TRACH3004412, TRACH3004456, TRACH3004537, TRACH3004596, TRACH3004651, TRACH3004652, TRACH3004671, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005274, TRACH3005479, TRACH3005549,

TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007627, TRACH3007689, TRACH3007766, TRACH3007866, TRACH3007995, TRACH3008042, TRACH3008061, TRACH3008201, TRACH3008508, TRACH3008629, TRACH3008632, TRACH3008688, TRACH3008697, TRACH3008887, TRACH3008902, TRACH3008990, TRACH3009001, TRACH3009059, TRACH3009061, TRACH3009148,

TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014415, TRACH3014580, TRACH3014641,

TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928,

TRACH3020930, TRACH3021023, TRACH3021066, TRACH3021212, TRACH3021335, TRACH3021373, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026542, TRACH3026650, TRACH3026676, TRACH3026744, TRACH3026949, TRACH3027229, TRACH3027527, TRACH3027681, TRACH3027701,

TRACH3027885, TRACH3028031, TRACH3028164, TRACH3028180, TRACH3028195, TRACH3028441, TRACH3028597, TRACH3028678, TRACH3028837, TRACH3028847, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488,

TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996,

TSTOM2002265, TSTOM2002505, TSTOM2002561, TSTOM2002611, TSTOM2002672, TSTOM2002682, TUTER1000014, TUTER1000137, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN1000186, UMVEN2000046, UMVEN2000069, UMVEN2000121, UMVEN2000133, UTERU1000008, UTERU1000015, UTERU1000024, UTERU1000031, UTERU1000032, UTERU1000057, UTERU1000065, UTERU1000077, UTERU1000093, UTERU1000096, UTERU1000109, UTERU1000131, UTERU1000138, UTERU1000148, UTERU1000160, UTERU1000183, UTERU1000187, UTERU1000192,

UTERU1000249, UTERU1000337, UTERU1000339, UTERU2000023, UTERU2000047, UTERU2000074, UTERU2000099, UTERU2000154, UTERU2000197, UTERU2000218, UTERU2000238, UTERU2000243, UTERU2000260, UTERU2000329, UTERU2000332, UTERU2000338, UTERU2000349, UTERU2000377, UTERU2000393, UTERU2000418, UTERU2000465, UTERU2000485, UTERU2000517, UTERU2000524, UTERU2000537, UTERU2000539, UTERU2000541, UTERU2000542, UTERU2000546, UTERU2000550, UTERU2000569, UTERU2000607, UTERU2000629, UTERU2000649, UTERU2000663, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747,

UTERU2001876, UTERU2002001, UTERU2002011, UTERU2002176, UTERU2002198, UTERU2002332, UTERU2002473, UTERU2002547, UTERU2002693, UTERU2002733, UTERU2002736, UTERU2002737, UTERU2002826, UTERU2002841, UTERU2002964, UTERU2002993, UTERU2003035, UTERU2003057, UTERU2003126, UTERU2003135, UTERU2003321, UTERU2003399, UTERU2003456, UTERU2003577, UTERU2003704, UTERU2003926, UTERU2003973, UTERU2004015, UTERU2004037, UTERU2004039, UTERU2004061, UTERU2004073, UTERU2004163, UTERU2004299, UTERU2004461, UTERU2004520, UTERU2004564, UTERU2004664, UTERU2004688, UTERU2004698, UTERU2004861, UTERU2004929, UTERU2005004, UTERU2005050, UTERU2005069, UTERU2005074, UTERU2005179, UTERU2005292, UTERU2005346, UTERU2005446,

UTERU2005449, UTERU2005450, UTERU2005533, UTERU2005593, UTERU2005601, UTERU2005621, UTERU2005664, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547, UTERU2006568, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639, UTERU2007724, UTERU2007942, UTERU2008018, UTERU2008019, UTERU2008027, UTERU2008040, UTERU2008077, UTERU2008130, UTERU2008347, UTERU2008426, UTERU2008516, UTERU2008561, UTERU2008705, UTERU2008707, UTERU2008747, UTERU2008785,

UTERU2008845, UTERU2008901, UTERU2008930, UTERU2008938, UTERU2008939, UTERU2008962, UTERU2009094, UTERU2009120, UTERU2009131, UTERU2009147, UTERU2009206, UTERU2009283, UTERU2009414, UTERU2009483, UTERU2009510, UTERU2009538, UTERU2009540, UTERU2009776, UTERU2009904, UTERU2009951, UTERU2010115, UTERU2010124, UTERU2010164, UTERU2010231, UTERU2010304, UTERU2010320, UTERU2010431, UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012252,

UTERU2012333, UTERU2012407, UTERU2012581, UTERU2012610, UTERU2012615, UTERU2012688, UTERU2012703, UTERU2012715, UTERU2012741, UTERU2012786, UTERU2012856, UTERU2012890, UTERU2012976, UTERU2013048, UTERU2013078, UTERU2013231, UTERU2013280, UTERU2013322, UTERU2013483, UTERU2013502, UTERU2013926, UTERU2013976, UTERU2014001, UTERU2014024, UTERU2014223, UTERU2014398, UTERU2014464, UTERU2014548, UTERU2014601, UTERU2014631, UTERU2014668, UTERU2014728, UTERU2014998, UTERU2015062, UTERU2015087, UTERU2015108, UTERU2015190, UTERU2015198, UTERU2015202, UTERU2015405, UTERU2015640, UTERU2015830, UTERU2015880, UTERU2016147, UTERU2016157, UTERU2016464, UTERU2016757, UTERU2016761, UTERU2016822, UTERU2016896,

UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018955, UTERU2019038, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491,

UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2029503, UTERU2029660, UTERU2029742, UTERU2029953, UTERU2030213, UTERU2030270,

UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791,

UTERU2037843, UTERU2038171, UTERU2038251, UTERU3000226, UTERU3000298, UTERU3000402, UTERU3000645, UTERU3000665, UTERU3000670, UTERU3000738, UTERU3000828, UTERU3000844, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003774, UTERU3003776, UTERU3004384, UTERU3004477, UTERU3004523, UTERU3004616,

UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010526, UTERU3010604,

UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the PANCR, PEBLM, PERIC, PLACE, PROST, PUAEN, and RECTM libraries were analyzed for expression frequency. The analysis results are described below. Each is a relative value, where the expression level over all tissues is taken as 100. The larger this value, the greater the level of expression.

TABLE 36

| Clone Name | PANCR | PEBLM | PERIC | PLACE | PROST | PUAEN | RECTM |
|---|---|---|---|---|---|---|---|
| 3NB691000085 | 0 | 0 | 0 | 0 | 30.703 | 0 | 0 |
| 3NB691000116 | 0 | 0 | 0 | 5.789 | 0 | 0 | 0 |
| 3NB691000129 | 0 | 0 | 0 | 0 | 0 | 0 | 54.934 |
| 3NB691000173 | 0 | 0 | 0 | 0 | 2.800 | 0 | 0 |
| 3NB692000281 | 0 | 1.655 | 1.483 | 0.392 | 0 | 0 | 0 |
| 3NB692000330 | 0 | 3.315 | 0 | 0 | 1.564 | 4.978 | 9.597 |
| 3NB692000374 | 0 | 0 | 0 | 1.930 | 0 | 0 | 0 |
| 3NB692000545 | 0 | 0 | 19.004 | 5.021 | 0 | 0 | 0 |
| 3NB692000912 | 0 | 7.063 | 3.165 | 0 | 0 | 2.651 | 0 |
| 3NB692001002 | 0 | 0 | 0 | 0 | 6.938 | 0 | 0 |
| 3NB692001022 | 0 | 9.352 | 0 | 0 | 0 | 0 | 0 |
| 3NB692001334 | 0 | 0 | 0 | 0 | 1.904 | 0 | 5.840 |
| 3NB692001528 | 0 | 0 | 0 | 0 | 8.342 | 0 | 0 |
| 3NB692001538 | 0 | 0 | 0 | 0 | 7.045 | 0 | 43.225 |
| 3NB692001557 | 0 | 45.597 | 0 | 0 | 0 | 0 | 0 |
| 3NB692001853 | 0 | 19.682 | 0 | 0 | 0 | 0 | 0 |
| ADIPS2000425 | 0 | 0 | 0 | 1.382 | 0 | 0 | 0 |
| ADRGL1000144 | 0 | 0 | 0 | 0 | 0 | 18.858 | 0 |
| ADRGL2000006 | 0 | 7.814 | 0 | 1.850 | 0 | 0 | 0 |
| ASTRO1000165 | 0 | 0 | 14.930 | 0 | 0 | 0 | 0 |
| ASTRO2000014 | 0 | 3.087 | 0 | 0 | 0 | 0 | 0 |
| ASTRO2000046 | 0 | 3.067 | 0 | 0 | 0 | 0 | 0 |
| ASTRO2000141 | 0 | 0 | 0 | 0 | 0 | 8.569 | 0 |
| ASTRO2000480 | 0 | 0 | 19.071 | 0 | 10.040 | 0 | 0 |
| ASTRO2000914 | 0 | 0 | 13.112 | 0 | 0 | 0 | 0 |
| ASTRO2001029 | 0 | 0 | 0 | 0 | 0 | 7.076 | 0 |
| ASTRO2001806 | 0 | 0 | 0 | 0 | 0 | 16.145 | 0 |
| ASTRO2001823 | 0 | 0 | 0 | 0 | 5.148 | 0 | 0 |
| ASTRO2004032 | 0 | 0 | 0 | 0 | 21.613 | 0 | 0 |
| ASTRO2004628 | 0 | 0 | 0 | 15.841 | 0 | 0 | 0 |
| ASTRO2005242 | 0 | 0 | 0 | 16.430 | 0 | 0 | 0 |
| ASTRO2005687 | 0 | 0 | 0 | 0 | 3.953 | 12.579 | 0 |
| ASTRO2005863 | 0 | 0 | 0 | 47.359 | 0 | 0 | 0 |
| ASTRO2006475 | 0 | 0 | 50.171 | 0 | 0 | 0 | 0 |
| ASTRO2006920 | 0 | 0 | 0 | 0 | 0 | 33.852 | 0 |
| ASTRO2007948 | 0 | 0 | 0 | 4.457 | 0 | 14.132 | 0 |
| ASTRO2008216 | 0 | 23.985 | 21.495 | 0 | 0 | 0 | 0 |
| ASTRO2010819 | 0 | 0 | 0 | 1.197 | 0 | 0 | 0 |
| ASTRO2012552 | 0 | 0 | 9.957 | 0 | 0 | 0 | 0 |
| ASTRO2013585 | 0 | 0 | 0 | 0.868 | 5.186 | 0 | 10.607 |
| ASTRO2015987 | 0 | 0 | 0 | 32.718 | 0 | 0 | 0 |
| ASTRO2016114 | 0 | 0 | 0 | 25.800 | 0 | 0 | 0 |
| ASTRO2016491 | 0 | 0 | 0 | 0.896 | 1.786 | 0 | 0 |
| ASTRO2018540 | 0 | 0 | 0 | 2.768 | 0 | 0 | 33.835 |
| BGGI12000054 | 0 | 0 | 3.173 | 0.419 | 0 | 33.224 | 0 |
| BGGI12000533 | 0 | 0 | 0 | 0 | 8.006 | 0 | 0 |
| BGGI12000616 | 0 | 0 | 6.023 | 0 | 0 | 0 | 0 |
| BGGI12000839 | 0 | 5.543 | 0 | 0 | 0 | 8.322 | 0 |
| BGGI12001097 | 0 | 0 | 0 | 0 | 0 | 12.682 | 0 |
| BGGI12001682 | 0 | 0 | 0 | 0 | 3.662 | 0 | 0 |
| BLADE2004722 | 0 | 0 | 0 | 17.812 | 0 | 0 | 0 |
| BLADE2007589 | 0 | 0 | 0 | 0 | 17.531 | 0 | 0 |
| BNGH41000098 | 0 | 0 | 0 | 0 | 0 | 28.605 | 0 |
| BNGH41000104 | 0 | 0 | 0 | 0 | 0 | 42.253 | 0 |
| BNGH41000153 | 0 | 0 | 0 | 0 | 16.664 | 0 | 0 |
| BNGH41000190 | 0 | 0 | 17.045 | 4.503 | 8.974 | 0 | 0 |
| BNGH42000360 | 0 | 0 | 0 | 0.560 | 0 | 0 | 0 |
| BNGH42000739 | 0 | 0 | 0 | 2.215 | 0 | 0 | 0 |
| BNGH42002487 | 0 | 0 | 0 | 16.657 | 0 | 0 | 0 |
| BNGH42005968 | 0 | 17.264 | 15.472 | 0 | 0 | 0 | 0 |
| BNGH42006135 | 0 | 0 | 0 | 3.525 | 0 | 3.725 | 0 |
| BNGH42007460 | 0 | 0 | 0 | 3.587 | 0 | 0 | 0 |
| BNGH42007709 | 0 | 5.191 | 3.101 | 1.639 | 0.816 | 0 | 0 |
| BNGH42007788 | 0 | 0 | 0 | 0 | 10.917 | 0 | 0 |
| BNGH42008603 | 0 | 0 | 0 | 0 | 0 | 16.323 | 0 |
| BNGH42008850 | 0 | 0 | 0 | 18.750 | 0 | 0 | 0 |
| BNGH42009025 | 0 | 0 | 0 | 0 | 4.944 | 0 | 0 |
| BRACE1000042 | 0 | 0 | 20.508 | 0 | 0 | 0 | 0 |
| BRACE1000070 | 0 | 0 | 0 | 0 | 6.340 | 0 | 0 |
| BRACE1000115 | 0 | 0 | 0 | 0.333 | 0 | 0 | 0 |
| BRACE1000131 | 0 | 0 | 0 | 0 | 3.694 | 0 | 0 |
| BRACE1000132 | 0 | 0 | 0 | 25.564 | 0 | 0 | 0 |
| BRACE1000159 | 0 | 0 | 12.437 | 0 | 0 | 0 | 0 |
| BRACE1000186 | 0 | 0 | 0 | 0 | 7.668 | 0 | 0 |
| BRACE1000239 | 0 | 0 | 0 | 35.265 | 0 | 0 | 0 |
| BRACE2000893 | 0 | 0 | 30.442 | 0 | 0 | 0 | 0 |
| BRACE2001143 | 0 | 0 | 0 | 0 | 5.268 | 0 | 0 |
| BRACE2001477 | 0 | 0 | 0 | 6.121 | 0 | 0 | 0 |
| BRACE2001497 | 0 | 0 | 0 | 3.316 | 0 | 0 | 0 |
| BRACE2001508 | 0 | 66.247 | 0 | 0 | 0 | 0 | 0 |
| BRACE2001673 | 0 | 0 | 0 | 0 | 0.831 | 0 | 0 |
| BRACE2001892 | 0 | 0 | 0 | 0 | 1.074 | 0 | 0 |
| BRACE2002091 | 0 | 0 | 0 | 0 | 0 | 0 | 45.947 |
| BRACE2002441 | 0 | 8.465 | 0 | 0 | 3.994 | 0 | 0 |
| BRACE2002606 | 0 | 0 | 0 | 0 | 0 | 90.728 | 0 |
| BRACE2002661 | 0 | 0 | 19.399 | 0 | 0 | 0 | 0 |
| BRACE2002677 | 0 | 0 | 13.004 | 3.436 | 0 | 0 | 0 |
| BRACE2002860 | 0 | 0 | 0 | 4.481 | 0 | 0 | 0 |
| BRACE2002861 | 0 | 0 | 0 | 4.618 | 0 | 0 | 0 |
| BRACE2003172 | 0 | 0 | 0 | 7.519 | 0 | 0 | 0 |
| BRACE2003217 | 0 | 0 | 29.036 | 0 | 0 | 0 | 0 |
| BRACE2003254 | 0 | 0 | 0 | 1.611 | 0 | 0 | 0 |
| BRACE2003628 | 0 | 0 | 0 | 0 | 24.194 | 0 | 0 |
| BRACE2003827 | 0 | 6.586 | 0 | 0 | 0 | 0 | 0 |
| BRACE2003944 | 0 | 0 | 34.870 | 0 | 1.836 | 5.842 | 0 |
| BRACE2005087 | 0 | 0 | 0 | 9.230 | 0 | 0 | 0 |
| BRACE2005434 | 0 | 0 | 0 | 0 | 2.445 | 0 | 0 |
| BRACE2005448 | 0 | 0 | 0 | 12.102 | 0 | 0 | 0 |
| BRACE2005937 | 0 | 0 | 0 | 21.500 | 0 | 0 | 0 |
| BRACE2006162 | 0 | 0 | 41.692 | 0 | 0 | 0 | 0 |
| BRACE2006258 | 0 | 0 | 0 | 0 | 0 | 4.622 | 0 |
| BRACE2006264 | 0 | 0 | 0 | 15.021 | 0 | 0 | 0 |
| BRACE2006319 | 0 | 0 | 23.616 | 0 | 0 | 0 | 0 |
| BRACE2006397 | 0 | 0 | 0 | 0.525 | 2.094 | 0 | 0 |
| BRACE2006514 | 0 | 0 | 0 | 0 | 0 | 4.902 | 0 |
| BRACE2006537 | 0 | 8.090 | 0 | 1.915 | 15.267 | 0 | 0 |
| BRACE2006547 | 0 | 0 | 0 | 0 | 53.061 | 0 | 0 |
| BRACE2006753 | 0 | 46.220 | 0 | 0 | 0 | 0 | 0 |
| BRACE2006900 | 0 | 0 | 0 | 0.916 | 0 | 0 | 0 |
| BRACE2007089 | 0 | 6.365 | 0 | 0 | 3.003 | 0 | 0 |
| BRACE2007191 | 0 | 0 | 0 | 4.890 | 2.436 | 0 | 0 |
| BRACE2007254 | 0 | 0 | 50.143 | 0 | 0 | 0 | 0 |
| BRACE2007518 | 0 | 0 | 0 | 4.039 | 0 | 0 | 0 |
| BRACE2007567 | 0 | 0 | 0 | 0 | 0 | 0 | 30.191 |
| BRACE2007671 | 0 | 0 | 10.414 | 0 | 10.966 | 8.724 | 0 |
| BRACE2007767 | 0 | 0 | 0 | 5.541 | 0 | 0 | 0 |
| BRACE2008358 | 0 | 0 | 0 | 4.033 | 16.072 | 0 | 0 |
| BRACE2008653 | 0 | 0 | 0 | 0.939 | 1.870 | 0 | 0 |
| BRACE2008706 | 0 | 0 | 0 | 0 | 11.652 | 0 | 0 |
| BRACE2008941 | 0 | 0 | 0 | 0 | 35.442 | 0 | 0 |
| BRACE2008968 | 0 | 0 | 0 | 0 | 6.186 | 0 | 0 |
| BRACE2009037 | 0 | 0 | 0 | 0 | 0 | 1.648 | 0 |
| BRACE2009291 | 0 | 0 | 8.910 | 0 | 0 | 0 | 0 |
| BRACE2009517 | 0 | 20.387 | 0 | 0 | 0 | 0 | 0 |
| BRACE2015287 | 0 | 0 | 0 | 0 | 0 | 0 | 49.965 |
| BRACE2016366 | 0 | 0 | 57.236 | 0 | 0 | 0 | 0 |
| BRACE2017397 | 0 | 0 | 0 | 8.395 | 0 | 0 | 0 |
| BRACE2017574 | 0 | 0 | 0 | 9.118 | 0 | 0 | 0 |
| BRACE2019147 | 0 | 16.098 | 0 | 0 | 0 | 0 | 0 |
| BRACE2022638 | 0 | 5.602 | 0 | 0 | 2.643 | 0 | 0 |
| BRACE2026836 | 0 | 0 | 46.481 | 0 | 0 | 0 | 0 |
| BRACE2037295 | 0 | 24.657 | 0 | 0 | 0 | 0 | 0 |
| BRACE2039624 | 0 | 0 | 0 | 0 | 3.431 | 0 | 0 |
| BRACE3000071 | 0 | 0 | 90.468 | 0 | 0 | 0 | 0 |
| BRACE3002184 | 0 | 0 | 0 | 47.691 | 0 | 0 | 0 |
| BRACE3003026 | 0 | 0 | 0 | 29.061 | 0 | 0 | 0 |
| BRACE3004371 | 0 | 0 | 0 | 6.686 | 0 | 0 | 0 |
| BRACE3004843 | 0 | 0 | 0 | 56.621 | 0 | 0 | 0 |
| BRACE3010428 | 0 | 8.604 | 0 | 2.037 | 0 | 0 | 0 |
| BRACE3010438 | 0 | 0 | 0 | 0 | 0 | 10.104 | 0 |
| BRACE3032631 | 0 | 0 | 0 | 22.445 | 0 | 0 | 0 |
| BRALZ2009482 | 0 | 0 | 0 | 5.952 | 0 | 0 | 8.085 |
| BRAMY2000354 | 0 | 0 | 9.256 | 0 | 0 | 0 | 29.898 |
| BRAMY2000893 | 0 | 0 | 0 | 0 | 0 | 0 | 33.110 |
| BRAMY2004363 | 0 | 0 | 0 | 0 | 0 | 11.536 | 0 |
| BRAMY2004521 | 0 | 0 | 0 | 4.218 | 0 | 0 | 0 |
| BRAMY2006366 | 0 | 0 | 0 | 16.395 | 0 | 0 | 0 |
| BRAMY2007078 | 0 | 28.583 | 0 | 0 | 0 | 21.457 | 0 |
| BRAMY2012091 | 0 | 0 | 40 | 0 | 0 | 0 | 0 |
| BRAMY2012536 | 0 | 4.339 | 0 | 0 | 0 | 0 | 0 |
| BRAMY2013590 | 0 | 0 | 0 | 38.449 | 0 | 0 | 0 |
| BRAMY2014813 | 0 | 72.515 | 0 | 0 | 0 | 0 | 0 |
| BRAMY2016009 | 0 | 0 | 0 | 9.307 | 0 | 0 | 0 |
| BRAMY2016706 | 0 | 0 | 0 | 0 | 17.578 | 0 | 0 |
| BRAMY2019554 | 0 | 0 | 0 | 0 | 2.830 | 0 | 0 |
| BRAMY2020427 | 0 | 4.451 | 0 | 2.108 | 0 | 0 | 0 |
| BRAMY2021139 | 0 | 0 | 0 | 45.546 | 0 | 0 | 0 |
| BRAMY2022786 | 0 | 0 | 0 | 2.221 | 0 | 0 | 0 |
| BRAMY2023719 | 0 | 44.940 | 0 | 0 | 0 | 0 | 0 |
| BRAMY2023939 | 0 | 0 | 0 | 10.878 | 0 | 0 | 0 |
| BRAMY2024827 | 0 | 0 | 0 | 0 | 0 | 0 | 43.406 |
| BRAMY2028491 | 0 | 0 | 0 | 63.850 | 0 | 0 | 0 |
| BRAMY2028740 | 0 | 0 | 0 | 0 | 3.302 | 0 | 0 |
| BRAMY2028796 | 0 | 0 | 0 | 2.645 | 0 | 0 | 0 |
| BRAMY2032087 | 0 | 0 | 0 | 2.155 | 0 | 0 | 0 |
| BRAMY2036459 | 0 | 0 | 0 | 0 | 0 | 0 | 41.005 |
| BRAMY2037147 | 0 | 0 | 0 | 63.850 | 0 | 0 | 0 |
| BRAMY2040592 | 0 | 0 | 0 | 0 | 12.349 | 0 | 0 |
| BRAMY3004377 | 0 | 0 | 0 | 0 | 2.941 | 4.680 | 0 |
| BRAMY3010008 | 0 | 0 | 0 | 1.036 | 0 | 0 | 0 |
| BRAWH1000007 | 0 | 0 | 0 | 0 | 0 | 50.702 | 0 |
| BRAWH1000040 | 0 | 5.172 | 0 | 0 | 0 | 0 | 0 |
| BRAWH1000093 | 0 | 3.467 | 0 | 1.642 | 0 | 2.602 | 0 |
| BRAWH2000460 | 0 | 4.587 | 0 | 1.086 | 2.164 | 3.444 | 0 |
| BRAWH2000488 | 0 | 0 | 0 | 0 | 35.754 | 0 | 0 |
| BRAWH2000503 | 0 | 0 | 0 | 36.922 | 0 | 0 | 0 |
| BRAWH2000588 | 0 | 0 | 0 | 0 | 8.898 | 7.079 | 0 |
| BRAWH2000633 | 0 | 0 | 0 | 0 | 0 | 7.480 | 0 |
| BRAWH2000697 | 0 | 0 | 0 | 3.600 | 0 | 5.708 | 0 |
| BRAWH2000839 | 0 | 3.087 | 0 | 0 | 1.457 | 4.635 | 0 |
| BRAWH2000924 | 0 | 0 | 0 | 0 | 0 | 1.619 | 0 |
| BRAWH2001092 | 0 | 1.321 | 0 | 0.313 | 0.623 | 2.975 | 3.824 |
| BRAWH2001141 | 0 | 0 | 0 | 0 | 18.022 | 0 | 0 |
| BRAWH2001166 | 0 | 0 | 0 | 0 | 11.195 | 0 | 0 |
| BRAWH2001171 | 0 | 0 | 4.160 | 1.099 | 0 | 0 | 0 |
| BRAWH2001439 | 0 | 0 | 0 | 0 | 3.649 | 0 | 0 |
| BRAWH2001484 | 0 | 0 | 0 | 18.909 | 0 | 0 | 0 |
| BRAWH2001671 | 0 | 0 | 0 | 0 | 13.485 | 0 | 0 |
| BRAWH2002679 | 0 | 0 | 0 | 0 | 33.575 | 0 | 0 |
| BRAWH2003025 | 0 | 0 | 0 | 0 | 10.753 | 0 | 0 |
| BRAWH2004095 | 0 | 0 | 0 | 2.104 | 0 | 0 | 0 |
| BRAWH2005225 | 0 | 0 | 0 | 0 | 20.557 | 32.708 | 0 |
| BRAWH2005661 | 0 | 0 | 0 | 0 | 5.669 | 0 | 0 |
| BRAWH2008219 | 0 | 0 | 0 | 0 | 51.103 | 0 | 0 |
| BRAWH2008706 | 0 | 0 | 0 | 5.117 | 0 | 0 | 0 |
| BRAWH2010136 | 0 | 0 | 0 | 46.991 | 0 | 0 | 0 |
| BRAWH2011343 | 0 | 0 | 0 | 12.485 | 0 | 0 | 0 |
| BRAWH2012162 | 0 | 0 | 0 | 0 | 63.940 | 0 | 0 |
| BRAWH2013748 | 0 | 0 | 0 | 0 | 0 | 0 | 58.070 |
| BRAWH2014188 | 0 | 5.381 | 0 | 8.919 | 0 | 4.040 | 0 |
| BRAWH2014934 | 0 | 0 | 0 | 64.025 | 0 | 0 | 0 |
| BRAWH2017685 | 0 | 0 | 0 | 0.903 | 0 | 2.864 | 0 |
| BRAWH2018526 | 0 | 4.626 | 0 | 0 | 2.183 | 3.473 | 0 |
| BRAWH3002574 | 0 | 0 | 0 | 0 | 23.446 | 0 | 0 |
| BRAWH3002853 | 0 | 0 | 4.982 | 1.316 | 5.246 | 0 | 0 |
| BRAWH3005146 | 0 | 0 | 0 | 0 | 0 | 28.570 | 0 |
| BRAWH3016271 | 0 | 0 | 0 | 0 | 0 | 30.435 | 0 |
| BRAWH3021574 | 0 | 0 | 0 | 8.364 | 0 | 0 | 0 |
| BRAWH3023172 | 0 | 0 | 6.169 | 0 | 0 | 0 | 0 |
| BRCAN1000168 | 0 | 0 | 0 | 2.511 | 5.003 | 0 | 0 |
| BRCAN2003944 | 0 | 0 | 0 | 0 | 0 | 4.767 | 0 |
| BRCAN2015371 | 0 | 0 | 0 | 0 | 38.874 | 0 | 0 |
| BRCAN2024572 | 0 | 0 | 0 | 4.861 | 0 | 0 | 0 |
| BRCAN2026197 | 0 | 0 | 0 | 0 | 0 | 8.810 | 0 |
| BRCAN2027150 | 0 | 76.615 | 0 | 0 | 0 | 0 | 0 |
| BRCAN2027364 | 0 | 0 | 0 | 5.285 | 0 | 0 | 0 |
| BRCAN2028036 | 0 | 0 | 0 | 0 | 0 | 13.623 | 0 |
| BRCAN2028355 | 0 | 0 | 9.952 | 0 | 0 | 0 | 0 |
| BRCOC1000040 | 0 | 0 | 0 | 5.248 | 0 | 0 | 0 |
| BRCOC2003187 | 0 | 0 | 0 | 0 | 0 | 0 | 17.111 |
| BRCOC2003213 | 0 | 0 | 0 | 0 | 36.816 | 0 | 0 |
| BRCOC2004175 | 0 | 0 | 65.535 | 0 | 0 | 0 | 0 |
| BRCOC2010123 | 0 | 0 | 0 | 1.950 | 0 | 0 | 0 |
| BRCOC2019255 | 0 | 0 | 0 | 30.447 | 0 | 0 | 0 |
| BRHIP2000534 | 0 | 2.573 | 0 | 0 | 1.214 | 3.862 | 0 |
| BRHIP2000553 | 0 | 0 | 0 | 4.573 | 0 | 0 | 0 |
| BRHIP2007305 | 0 | 0 | 0 | 2.015 | 0 | 6.388 | 0 |
| BRHIP2015245 | 0 | 0 | 0 | 4.225 | 0 | 0 | 0 |
| BRHIP2015679 | 0 | 0 | 0 | 8.929 | 0 | 0 | 0 |
| BRHIP2021762 | 0 | 0 | 0 | 4.958 | 0 | 0 | 0 |
| BRHIP2021870 | 0 | 33.373 | 0 | 0 | 0 | 0 | 0 |
| BRHIP2022326 | 0 | 0 | 5.731 | 0 | 0 | 0 | 0 |
| BRHIP2022986 | 0 | 21.388 | 0 | 5.064 | 0 | 32.111 | 0 |
| BRHIP2023229 | 0 | 0 | 0 | 0 | 0 | 84.688 | 0 |
| BRHIP2023438 | 0 | 0 | 0 | 45.400 | 0 | 0 | 0 |
| BRHIP2023695 | 0 | 0 | 0 | 63.562 | 0 | 0 | 0 |
| BRHIP2023762 | 0 | 17.938 | 0 | 0 | 0 | 0 | 0 |
| BRHIP2023888 | 0 | 0 | 0 | 3.274 | 1.305 | 2.076 | 0 |
| BRHIP2027077 | 0 | 15.182 | 0 | 0 | 0 | 0 | 0 |
| BRHIP3000111 | 0 | 0 | 0 | 0 | 0 | 5.777 | 0 |
| BRHIP3000131 | 0 | 0 | 0 | 6.535 | 0 | 20.721 | 0 |
| BRHIP3003961 | 0 | 0 | 0 | 33.755 | 0 | 0 | 0 |
| BRHIP3004215 | 0 | 0 | 0 | 24.161 | 0 | 0 | 0 |
| BRHIP3020046 | 0 | 7.614 | 0 | 0 | 0 | 0 | 0 |
| BRHIP3021987 | 0 | 0 | 0 | 63.562 | 0 | 0 | 0 |
| BRSSN2000498 | 0 | 16.450 | 0 | 0 | 0 | 0 | 0 |
| BRSSN2001869 | 0 | 22.689 | 0 | 0 | 0 | 0 | 0 |
| BRSSN2003012 | 0 | 0 | 0 | 7.699 | 0 | 0 | 0 |
| BRSSN2011262 | 0 | 0 | 18.984 | 0 | 0 | 0 | 0 |
| BRSSN2014610 | 0 | 0 | 0 | 12.708 | 0 | 0 | 0 |
| BRSSN2015982 | 0 | 0 | 0 | 28.913 | 0 | 0 | 0 |
| BRSTN1000083 | 0 | 0 | 4.301 | 2.273 | 0 | 0 | 0 |
| BRSTN2004863 | 0 | 0 | 0 | 17.731 | 0 | 0 | 0 |
| BRSTN2010089 | 0 | 0 | 0 | 11.742 | 0 | 0 | 0 |
| BRSTN2011961 | 0 | 0 | 3.902 | 1.031 | 1.027 | 3.268 | 0 |
| BRSTN2012069 | 0 | 0.355 | 0.446 | 0.219 | 0.403 | 0.107 | 3.704 |
| BRSTN2012174 | 0 | 1.520 | 0 | 0.720 | 0 | 0 | 0 |
| BRSTN2013502 | 0 | 0 | 0 | 0 | 0 | 0 | 52.880 |
| BRSTN2014490 | 0 | 0 | 0 | 0 | 32.674 | 0 | 0 |
| BRSTN2015889 | 0 | 0 | 0 | 0 | 13.737 | 0 | 0 |
| BRSTN2016470 | 0 | 0 | 0 | 1.169 | 0.776 | 0 | 0 |
| BRSTN2017104 | 0 | 0 | 0 | 0 | 6.915 | 0 | 0 |
| BRTHA1000311 | 0 | 0 | 0 | 5.251 | 0 | 0 | 0 |
| BRTHA2002726 | 0 | 0 | 0 | 0 | 76.179 | 0 | 0 |
| BRTHA2002808 | 0 | 87.143 | 0 | 0 | 0 | 0 | 0 |
| BRTHA2005864 | 0 | 0 | 0 | 5.775 | 0 | 0 | 0 |
| BRTHA2007998 | 0 | 0 | 0 | 0 | 0 | 4.866 | 0 |
| BRTHA2012392 | 0 | 0 | 0 | 61.610 | 0 | 0 | 0 |
| BRTHA2016765 | 0 | 0 | 79.260 | 0 | 0 | 0 | 0 |
| BRTHA2027229 | 0 | 0 | 0 | 0 | 0 | 5.165 | 0 |
| BRTHA3003490 | 0 | 0 | 0 | 0 | 0 | 9.160 | 0 |
| BRTHA3003736 | 0 | 0 | 0 | 0 | 7.629 | 0 | 0 |
| BRTHA3007472 | 0 | 29.330 | 0 | 0 | 0 | 22.018 | 0 |
| BRTHA3007922 | 0 | 0 | 0 | 27.774 | 0 | 0 | 0 |
| BRTHA3010135 | 0 | 0 | 0 | 35.611 | 0 | 0 | 0 |
| BRTHA3011265 | 0 | 0 | 0 | 15.184 | 0 | 0 | 0 |
| BRTHA3021971 | 0 | 14.590 | 0 | 0 | 0 | 0 | 0 |
| CERVX2002770 | 0 | 0 | 0 | 0 | 0 | 0 | 51.288 |
| CHONS2002829 | 0 | 0 | 0 | 5.815 | 0 | 0 | 0 |
| COLON2002443 | 0 | 33.895 | 0 | 0 | 0 | 0 | 0 |
| CTONG1000009 | 0 | 0 | 0 | 8.644 | 0 | 0 | 0 |
| CTONG1000052 | 0 | 0 | 4.070 | 1.075 | 2.143 | 0 | 0 |
| CTONG1000094 | 0 | 0 | 2.623 | 0 | 1.381 | 2.198 | 0 |
| CTONG1000175 | 0 | 0 | 0 | 2.996 | 11.942 | 0 | 0 |
| CTONG1000218 | 0 | 0 | 3.244 | 0 | 1.708 | 0 | 0 |
| CTONG1000220 | 0 | 0 | 0 | 0 | 0 | 75.183 | 0 |
| CTONG1000277 | 0 | 0 | 0 | 36.809 | 0 | 0 | 0 |
| CTONG1000467 | 0 | 0 | 0 | 15.292 | 0 | 0 | 0 |
| CTONG2000042 | 0 | 0 | 0 | 31.174 | 0 | 0 | 0 |
| CTONG2000063 | 0 | 0 | 0 | 3.810 | 0 | 0 | 0 |
| CTONG2000411 | 0 | 0 | 0 | 10.242 | 0 | 0 | 0 |
| CTONG2000452 | 0 | 0 | 0 | 5.183 | 0 | 16.434 | 0 |
| CTONG2000469 | 0 | 0 | 0 | 0 | 31.068 | 0 | 0 |
| CTONG2000561 | 0 | 0 | 0 | 38.747 | 0 | 0 | 0 |
| CTONG2000589 | 0 | 0 | 0 | 39.688 | 39.543 | 0 | 0 |
| CTONG2001320 | 0 | 0 | 0 | 0 | 0 | 29.748 | 0 |
| CTONG2001513 | 0 | 0 | 0 | 48.862 | 0 | 0 | 0 |
| CTONG2001748 | 0 | 0 | 0 | 3.720 | 0 | 0 | 0 |
| CTONG2001932 | 0 | 0 | 0 | 18.386 | 0 | 0 | 0 |
| CTONG2002073 | 0 | 0 | 0 | 2.607 | 5.194 | 0 | 0 |
| CTONG2002270 | 0 | 0 | 0 | 2.832 | 0 | 0 | 0 |
| CTONG2002453 | 0 | 0 | 0 | 0 | 0 | 43.805 | 0 |
| CTONG2002520 | 0 | 0 | 0 | 5.239 | 0 | 0 | 0 |
| CTONG2002709 | 0 | 0 | 0 | 0 | 15.368 | 0 | 0 |
| CTONG2002744 | 0 | 0 | 0 | 1.048 | 0 | 0 | 0 |
| CTONG2002903 | 0 | 0 | 0 | 5.482 | 10.924 | 0 | 0 |
| CTONG2003094 | 0 | 0 | 12.342 | 3.261 | 0 | 0 | 0 |
| CTONG2005553 | 0 | 0 | 0 | 1.274 | 0 | 0 | 0 |
| CTONG2005913 | 0 | 0 | 0 | 0 | 15.825 | 0 | 0 |
| CTONG2006562 | 0 | 0 | 0 | 2.492 | 0 | 0 | 0 |
| CTONG2006709 | 0 | 0 | 0 | 9.205 | 0 | 14.592 | 0 |
| CTONG2006932 | 0 | 0 | 0 | 0 | 3.903 | 0 | 0 |
| CTONG2007072 | 0 | 16.170 | 14.491 | 0 | 0 | 0 | 0 |
| CTONG2007078 | 0 | 19.211 | 0 | 0 | 0 | 0 | 0 |
| CTONG2007500 | 0 | 0 | 0 | 2.677 | 0 | 0 | 0 |
| CTONG2007613 | 0 | 0 | 0 | 0 | 0 | 49.390 | 0 |
| CTONG2007776 | 0 | 0 | 0 | 0 | 15.083 | 0 | 0 |
| CTONG2008398 | 0 | 0 | 0 | 0 | 0 | 4.314 | 0 |
| CTONG2008506 | 0 | 0 | 0 | 1.675 | 0 | 5.310 | 0 |
| CTONG2008518 | 0 | 0 | 0 | 6.854 | 0 | 0 | 0 |
| CTONG2010348 | 0 | 0 | 0 | 3.151 | 0 | 0 | 0 |
| CTONG2010821 | 0 | 5.371 | 0 | 0 | 2.534 | 0 | 0 |
| CTONG2011825 | 0 | 0 | 0 | 0 | 0 | 48.443 | 0 |
| CTONG2012001 | 0 | 0 | 0 | 12.726 | 0 | 0 | 0 |
| CTONG2012401 | 0 | 0 | 28.336 | 0 | 0 | 23.736 | 0 |
| CTONG2012452 | 0 | 2.677 | 0 | 1.901 | 0 | 0 | 0 |
| CTONG2012607 | 0 | 0 | 0 | 0 | 0 | 0 | 37.630 |
| CTONG2012847 | 0 | 0 | 0 | 0 | 6.107 | 9.717 | 0 |
| CTONG2012879 | 0 | 0 | 11.453 | 0 | 0 | 0 | 0 |
| CTONG2013149 | 0 | 0 | 0 | 0 | 0 | 75.183 | 0 |
| CTONG2013156 | 0 | 0 | 0 | 36.809 | 0 | 0 | 0 |
| CTONG2013222 | 0 | 0 | 0 | 0 | 13.327 | 0 | 0 |
| CTONG2015091 | 0 | 0 | 5.470 | 0 | 0 | 0 | 0 |
| CTONG2016056 | 0 | 0 | 13.394 | 0 | 0 | 0 | 0 |
| CTONG2016575 | 0 | 0 | 0 | 2.654 | 0 | 8.416 | 0 |
| CTONG2018383 | 0 | 33.978 | 0 | 0 | 0 | 0 | 0 |
| CTONG2018632 | 0 | 0 | 0 | 0 | 0 | 45.130 | 0 |
| CTONG2019029 | 0 | 0 | 0 | 14.402 | 0 | 0 | 0 |
| CTONG2019248 | 0 | 0 | 0 | 26.925 | 0 | 0 | 0 |
| CTONG2019590 | 0 | 0 | 0 | 19.151 | 0 | 0 | 0 |
| CTONG2020445 | 0 | 0 | 0 | 37.458 | 0 | 0 | 0 |
| CTONG2020484 | 0 | 0 | 0 | 0 | 0 | 6.634 | 0 |
| CTONG2020560 | 0 | 0 | 0 | 5.340 | 0 | 0 | 0 |
| CTONG2020582 | 0 | 0 | 12.925 | 13.659 | 6.804 | 0 | 0 |
| CTONG2024031 | 0 | 0 | 0 | 9.199 | 0 | 0 | 56.233 |
| CTONG2028124 | 0 | 3.778 | 3.385 | 0 | 7.129 | 0 | 0 |
| CTONG2028208 | 0 | 0 | 23.853 | 0 | 0 | 0 | 0 |
| CTONG2028309 | 0 | 0 | 0 | 13.063 | 0 | 0 | 0 |
| CTONG3000657 | 0 | 0 | 0 | 0 | 13.759 | 0 | 0 |
| CTONG3001420 | 0 | 0 | 0 | 48.862 | 0 | 0 | 0 |
| CTONG3002674 | 0 | 0 | 0 | 39.760 | 0 | 0 | 0 |
| CTONG3002835 | 0 | 0 | 7.401 | 0 | 0 | 0 | 0 |
| CTONG3004726 | 0 | 0 | 0 | 30.140 | 0 | 0 | 0 |
| CTONG3008894 | 0 | 0 | 0 | 1.294 | 0 | 0 | 0 |
| CTONG3009239 | 0 | 0 | 9.726 | 0 | 0 | 8.147 | 0 |
| D30ST1000109 | 0 | 13.416 | 0 | 0 | 0 | 0 | 0 |
| D30ST1000270 | 0 | 0 | 0 | 0 | 9.163 | 14.579 | 0 |
| D30ST2002068 | 0 | 8.074 | 0 | 0.382 | 0 | 0 | 0 |
| D30ST2002182 | 0 | 0 | 0 | 13.227 | 0 | 0 | 0 |
| D30ST3000169 | 0 | 11.035 | 0 | 0 | 0 | 0 | 0 |
| DFNES1000083 | 0 | 0 | 0 | 0 | 0 | 38.078 | 0 |
| DFNES1000150 | 0 | 0 | 0 | 3.975 | 0 | 0 | 0 |
| DFNES1000185 | 0 | 0 | 0 | 0 | 0 | 7.442 | 0 |
| DFNES2000011 | 0 | 0 | 0 | 1.946 | 0 | 0 | 0 |
| DFNES2000268 | 0 | 0 | 0 | 0 | 0 | 6.231 | 0 |
| DFNES2000292 | 0 | 0 | 0 | 0 | 0 | 42.353 | 0 |
| DFNES2000432 | 0 | 56.052 | 0 | 0 | 0 | 0 | 0 |
| DFNES2000457 | 0 | 0 | 0 | 0 | 31.500 | 0 | 0 |
| DFNES2003192 | 0 | 0 | 0 | 5.331 | 0 | 0 | 0 |
| DFNES2004371 | 0 | 0 | 0 | 5.616 | 0 | 0 | 0 |
| DFNES2005540 | 0 | 0 | 0 | 0 | 0 | 17.057 | 0 |
| DFNES2007634 | 0 | 0 | 0 | 3.164 | 0 | 0 | 0 |
| DFNES2011239 | 0 | 0 | 0 | 0 | 0 | 42.161 | 0 |
| DFNES2011499 | 0 | 0 | 0 | 11.041 | 0 | 0 | 0 |
| FCBBF1000024 | 0 | 0 | 0 | 3.907 | 0 | 0 | 0 |
| FCBBF1000025 | 0 | 0 | 0 | 0 | 0 | 18.057 | 0 |
| FCBBF1000027 | 0 | 2.753 | 0 | 0 | 1.299 | 0 | 0 |
| FCBBF1000053 | 0 | 20.343 | 0 | 0 | 0 | 0 | 0 |
| FCBBF1000061 | 0 | 0 | 0 | 4.186 | 0 | 0 | 0 |
| FCBBF1000077 | 0 | 0 | 0 | 0 | 0 | 4.570 | 0 |
| FCBBF1000139 | 0 | 0 | 0 | 3.111 | 0 | 0 | 0 |
| FCBBF1000182 | 0 | 0 | 0 | 15.092 | 0 | 0 | 0 |
| FCBBF1000197 | 0 | 8.030 | 0 | 9.506 | 0 | 0 | 0 |
| FCBBF1000294 | 0 | 8.093 | 0 | 3.832 | 0 | 0 | 0 |
| FCBBF1000367 | 0 | 0 | 10.233 | 0 | 0 | 0 | 33.054 |
| FCBBF1000412 | 0 | 14.546 | 0 | 3.444 | 0 | 10.920 | 0 |
| FCBBF1000425 | 0 | 39.241 | 0 | 0 | 0 | 0 | 0 |
| FCBBF1000449 | 0 | 50.253 | 0 | 0 | 0 | 0 | 0 |
| FCBBF1000466 | 0 | 0 | 4.290 | 0 | 2.258 | 0 | 0 |
| FCBBF1000563 | 0 | 0 | 0 | 0 | 0 | 8.475 | 0 |
| FCBBF2000502 | 0 | 49.587 | 0 | 0 | 0 | 0 | 0 |
| FCBBF2000566 | 0 | 27.938 | 0 | 0 | 0 | 0 | 0 |
| FCBBF2000591 | 0 | 2.155 | 0 | 0 | 0 | 0 | 0 |
| FCBBF2000685 | 0 | 0 | 0 | 0 | 0 | 38.388 | 0 |
| FCBBF2000885 | 0 | 0 | 0 | 5.464 | 0 | 0 | 0 |
| FCBBF2001105 | 0 | 0 | 0 | 0 | 21.530 | 0 | 0 |
| FCBBF2001309 | 0 | 0 | 0 | 7.743 | 0 | 0 | 0 |
| FCBBF2001538 | 0 | 0 | 5.132 | 0 | 2.702 | 0 | 0 |
| FCBBF2002349 | 0 | 0 | 26.593 | 0 | 0 | 0 | 0 |
| FCBBF2005637 | 0 | 0 | 18.719 | 0 | 0 | 0 | 0 |
| FCBBF2006634 | 0 | 0 | 0 | 16.801 | 0 | 0 | 0 |
| FCBBF3000228 | 0 | 0 | 0 | 15.949 | 0 | 0 | 0 |
| FCBBF3000434 | 0 | 0 | 0 | 0 | 6.566 | 0 | 0 |
| FCBBF3001081 | 0 | 0 | 17.687 | 9.346 | 0 | 0 | 0 |
| FCBBF3001657 | 0 | 0 | 15.891 | 0 | 0 | 0 | 0 |
| FCBBF3001890 | 0 | 0 | 0 | 0 | 9.755 | 0 | 0 |
| FCBBF3004021 | 0 | 80.271 | 0 | 0 | 0 | 0 | 0 |
| FCBBF3004261 | 0 | 0 | 0 | 16.099 | 0 | 0 | 0 |
| FCBBF3004473 | 0 | 0 | 0 | 0 | 8.149 | 0 | 0 |
| FCBBF3005320 | 0 | 0 | 0 | 0 | 9.674 | 0 | 0 |
| FCBBF3005330 | 0 | 5.207 | 0 | 0 | 0 | 0 | 0 |
| FCBBF3005497 | 0 | 5.909 | 0 | 2.798 | 0 | 8.872 | 0 |
| FCBBF3008644 | 0 | 64.763 | 0 | 0 | 0 | 0 | 0 |
| FCBBF3009541 | 0 | 0 | 0 | 1.467 | 0 | 2.326 | 0 |
| FCBBF3014260 | 0 | 0 | 0 | 4.018 | 0 | 0 | 0 |
| FCBBF3014770 | 0 | 0 | 64.279 | 0 | 0 | 0 | 0 |
| FCBBF3017024 | 0 | 38.525 | 0 | 18.243 | 0 | 0 | 0 |
| FCBBF3021191 | 0 | 0 | 0 | 0 | 65.749 | 0 | 0 |
| FCBBF3023667 | 0 | 0.998 | 0 | 0.945 | 0.471 | 0 | 0 |
| FCBBF3028244 | 0 | 0 | 6.254 | 0 | 0 | 0 | 0 |
| FCBBF4000446 | 0 | 0 | 0 | 1.337 | 0 | 0 | 0 |
| FEBRA2000105 | 0 | 0 | 0 | 2.553 | 0 | 0 | 0 |
| FEBRA2000402 | 0 | 0 | 0 | 0 | 19.655 | 0 | 0 |
| FEBRA2000404 | 0 | 0 | 67.776 | 0 | 0 | 0 | 0 |
| FEBRA2000510 | 0 | 0 | 0 | 0 | 0 | 20.820 | 0 |
| FEBRA2000581 | 0 | 0 | 0 | 0 | 0 | 4.489 | 0 |
| FEBRA2000733 | 0 | 1.267 | 2.270 | 1.200 | 1.195 | 0.951 | 0 |
| FEBRA2000787 | 0 | 0 | 0 | 3.515 | 0 | 0 | 0 |
| FEBRA2000881 | 0 | 0 | 0 | 5.633 | 0 | 0 | 0 |
| FEBRA2002933 | 0 | 0 | 0 | 0 | 2.625 | 2.088 | 8.052 |
| FEBRA2003100 | 0 | 0 | 0 | 1.172 | 0 | 0 | 0 |
| FEBRA2003750 | 0 | 0 | 0 | 3.249 | 0 | 0 | 0 |
| FEBRA2003833 | 0 | 0 | 0 | 6.155 | 0 | 0 | 0 |
| FEBRA2003926 | 0 | 0 | 0 | 0 | 2.476 | 3.939 | 0 |
| FEBRA2003930 | 0 | 13.509 | 0 | 0 | 0 | 0 | 0 |
| FEBRA2004042 | 0 | 0 | 0 | 3.775 | 0 | 0 | 0 |
| FEBRA2004293 | 0 | 0 | 0 | 0 | 18.855 | 0 | 0 |
| FEBRA2004325 | 0 | 0 | 0 | 6.268 | 0 | 39.749 | 0 |
| FEBRA2004412 | 0 | 0 | 0 | 5.225 | 0 | 0 | 0 |
| FEBRA2004628 | 0 | 0 | 0 | 0 | 10.116 | 0 | 0 |
| FEBRA2004818 | 0 | 0 | 0 | 2.423 | 0 | 0 | 0 |
| FEBRA2005416 | 0 | 0 | 0 | 1.911 | 0 | 6.059 | 0 |
| FEBRA2005572 | 0 | 0 | 0 | 3.171 | 0 | 0 | 0 |
| FEBRA2005734 | 0 | 0 | 0 | 0 | 2.994 | 0 | 0 |
| FEBRA2005824 | 0 | 2.804 | 0 | 0 | 0 | 0 | 0 |
| FEBRA2006055 | 0 | 0 | 0 | 4.701 | 0 | 0 | 57.471 |
| FEBRA2006112 | 0 | 0 | 0 | 23.494 | 0 | 0 | 0 |
| FEBRA2006270 | 0 | 0 | 0 | 2.080 | 0 | 0 | 0 |
| FEBRA2006372 | 0 | 0 | 0 | 0 | 0 | 7.578 | 0 |
| FEBRA2006519 | 0 | 0 | 0 | 21.163 | 0 | 0 | 0 |
| FEBRA2006553 | 0 | 0 | 0 | 0 | 14.030 | 0 | 0 |
| FEBRA2007212 | 0 | 29.506 | 0 | 0 | 0 | 0 | 0 |
| FEBRA2008081 | 0 | 0 | 0 | 3.781 | 0 | 0 | 0 |
| FEBRA2008087 | 0 | 2.695 | 0 | 1.276 | 1.271 | 0 | 7.800 |
| FEBRA2008255 | 0 | 0 | 0 | 9.006 | 0 | 0 | 0 |
| FEBRA2008302 | 0 | 0 | 0 | 1.894 | 0 | 0 | 0 |
| FEBRA2009276 | 0 | 0 | 0 | 41.654 | 0 | 0 | 0 |
| FEBRA2009419 | 0 | 9.932 | 0 | 4.703 | 0 | 7.455 | 0 |
| FEBRA2009541 | 0 | 0 | 0 | 29.601 | 0 | 0 | 0 |
| FEBRA2012397 | 0 | 0 | 0 | 41.654 | 0 | 0 | 0 |
| FEBRA2014417 | 0 | 0 | 0 | 0.961 | 0 | 3.047 | 0 |
| FEBRA2018476 | 0 | 0 | 7.360 | 0 | 7.750 | 0 | 0 |
| FEBRA2022055 | 0 | 0 | 3.584 | 0 | 7.548 | 3.002 | 0 |
| FEBRA2022504 | 0 | 0 | 0 | 0 | 5.824 | 0 | 0 |
| FEBRA2023927 | 0 | 0 | 0 | 7.249 | 0 | 0 | 0 |
| FEBRA2025838 | 0 | 6.175 | 0 | 0 | 0 | 0 | 0 |
| FELIV1000153 | 0 | 1.240 | 1.111 | 0 | 0 | 0 | 0 |
| HCASM2000363 | 0 | 5.112 | 0 | 0 | 2.412 | 3.837 | 0 |
| HCHON1000030 | 0 | 1.363 | 1.222 | 0.646 | 5.146 | 0 | 0 |
| HCHON1000166 | 0 | 0 | 0 | 0 | 10.668 | 0 | 0 |
| HCHON2000028 | 0 | 0 | 0 | 14.118 | 0 | 0 | 0 |
| HCHON2000056 | 0 | 0 | 0 | 0 | 2.012 | 1.600 | 0 |
| HCHON2000062 | 0 | 0 | 0 | 7.459 | 0 | 0 | 0 |
| HCHON2000087 | 0 | 0 | 0 | 0 | 0 | 3.491 | 0 |
| HCHON2000160 | 0 | 7.566 | 0 | 0 | 0 | 0 | 0 |
| HCHON2000226 | 0 | 0 | 0 | 0.718 | 0 | 0 | 8.774 |
| HCHON2000244 | 0 | 0 | 0 | 0 | 4.666 | 3.712 | 0 |
| HCHON2000265 | 0 | 0 | 13.629 | 0 | 0 | 0 | 0 |
| HCHON2000295 | 0 | 0 | 0 | 0.780 | 0 | 0 | 0 |
| HCHON2000322 | 0 | 0 | 0 | 19.541 | 0 | 0 | 0 |
| HCHON2000373 | 0 | 0 | 0 | 0 | 0 | 5.719 | 0 |
| HCHON2000475 | 0 | 10.005 | 0 | 0 | 0 | 0 | 0 |
| HCHON2000508 | 0 | 2.807 | 0 | 1.994 | 1.324 | 0 | 0 |
| HCHON2000826 | 0 | 0 | 0 | 0 | 2.538 | 4.038 | 0 |
| HCHON2001039 | 0 | 0 | 0 | 1.986 | 0 | 0 | 0 |
| HCHON2001084 | 0 | 0 | 0 | 0 | 8.143 | 0 | 0 |
| HCHON2001099 | 0 | 0 | 0 | 0 | 9.673 | 0 | 0 |
| HCHON2001200 | 0 | 3.251 | 2.913 | 0.770 | 1.534 | 0 | 0 |
| HCHON2001217 | 0 | 0 | 0 | 0 | 0 | 11.746 | 0 |
| HCHON2001359 | 0 | 6.399 | 5.735 | 0 | 0 | 0 | 0 |
| HCHON2001407 | 0 | 0 | 0 | 0 | 5.601 | 0 | 0 |
| HCHON2001434 | 0 | 0 | 0 | 0 | 5.417 | 0 | 0 |
| HCHON2001505 | 0 | 0 | 0 | 15.995 | 0 | 0 | 0 |
| HCHON2004002 | 0 | 23.605 | 0 | 1.397 | 0 | 0 | 0 |
| HCHON2005921 | 0 | 0 | 0 | 7.381 | 0 | 0 | 0 |
| HEART1000074 | 0 | 0 | 0 | 1.351 | 0 | 0 | 0 |
| HEART1000088 | 0 | 0 | 50.241 | 0 | 0 | 0 | 0 |
| HEART1000139 | 0 | 0 | 33.548 | 0 | 0 | 0 | 0 |
| HEART1000142 | 0 | 0 | 36.026 | 0 | 0 | 0 | 0 |
| HEART1000149 | 0 | 0 | 0 | 1.671 | 16.652 | 0 | 20.432 |
| HEART1000185 | 0 | 0 | 0 | 0 | 2.804 | 0 | 0 |
| HEART2000506 | 0 | 0 | 0 | 3.121 | 0 | 0 | 0 |
| HEART2000520 | 0 | 0 | 19.426 | 0 | 0 | 0 | 0 |
| HEART2000541 | 0 | 0 | 27.038 | 0 | 0 | 0 | 0 |
| HEART2003836 | 0 | 0 | 27.716 | 0 | 0 | 0 | 0 |
| HEART2006521 | 0 | 17.978 | 0 | 0 | 8.482 | 0 | 0 |
| HEART2007696 | 0 | 0 | 33.551 | 0 | 0 | 0 | 0 |
| HEART2010495 | 0 | 0 | 50.241 | 0 | 0 | 0 | 0 |
| HHDPC1000083 | 0 | 0 | 6.593 | 1.742 | 3.471 | 0 | 0 |
| HHDPC1000163 | 0 | 0 | 0 | 0 | 4.460 | 0 | 0 |
| HHDPC2000055 | 0 | 0 | 0 | 3.254 | 0 | 0 | 0 |
| HHDPC2000115 | 0 | 0 | 0 | 0 | 9.277 | 0 | 0 |
| HHDPC2000149 | 0 | 0 | 0 | 7.952 | 0 | 0 | 0 |
| HHDPC2000455 | 0 | 0 | 0 | 0.978 | 0 | 0 | 0 |
| HHDPC2000456 | 0 | 0 | 0 | 5.526 | 0 | 8.761 | 0 |
| HHDPC2003439 | 0 | 0 | 0 | 0.453 | 0 | 1.435 | 0 |
| HHDPC2003983 | 0 | 0 | 1.876 | 1.487 | 0 | 0 | 0 |
| HHDPC2007267 | 0 | 0 | 0 | 12.164 | 13.851 | 0 | 0 |
| HLUNG1000055 | 0 | 0 | 0 | 1.927 | 0 | 3.054 | 0 |
| HLUNG1000076 | 0 | 0 | 0 | 10.480 | 2.983 | 61.709 | 0 |
| HLUNG2000315 | 0 | 0 | 0 | 27.144 | 0 | 0 | 0 |
| HLUNG2000412 | 0 | 0 | 0 | 4.765 | 9.496 | 0 | 0 |
| HLUNG2000501 | 0 | 0 | 0 | 0 | 0 | 0 | 18.665 |
| HLUNG2000884 | 0 | 16.089 | 0 | 3.809 | 7.591 | 0 | 0 |
| HLUNG2001459 | 0 | 49.262 | 0 | 11.664 | 0 | 0 | 0 |
| HLUNG2001518 | 0 | 0 | 0 | 1.946 | 11.634 | 0 | 0 |
| HLUNG2001633 | 0 | 0 | 0 | 42.749 | 0 | 0 | 0 |
| HLUNG2001712 | 0 | 0 | 0 | 0 | 11.693 | 0 | 0 |
| HLUNG2002059 | 0 | 0 | 0 | 29.250 | 0 | 0 | 0 |
| HLUNG2002648 | 0 | 0 | 0 | 0 | 0 | 20.395 | 0 |
| HLUNG2003042 | 0 | 0 | 0 | 4.236 | 0 | 0 | 0 |
| HLUNG2003306 | 0 | 0 | 0 | 22.788 | 0 | 0 | 0 |
| HLUNG2003778 | 0 | 38.774 | 0 | 0 | 0 | 0 | 0 |
| HLUNG2004159 | 0 | 13.628 | 0 | 3.227 | 0 | 10.230 | 0 |
| HLUNG2004170 | 0 | 0 | 0 | 43.864 | 0 | 0 | 0 |
| HLUNG2005230 | 0 | 17.182 | 0 | 4.068 | 0 | 0 | 0 |
| HLUNG2005738 | 0 | 0 | 0 | 32.670 | 0 | 0 | 0 |
| HLUNG2008637 | 0 | 0 | 0 | 0 | 5.991 | 0 | 0 |
| HLUNG2009215 | 0 | 0 | 0 | 0 | 49.159 | 0 | 0 |
| HLUNG2011298 | 0 | 0 | 64.746 | 0 | 0 | 0 | 0 |
| HLUNG2017262 | 0 | 0 | 0 | 11.289 | 0 | 0 | 0 |
| HSYRA1000062 | 0 | 0 | 0 | 0.762 | 3.038 | 0 | 0 |
| HSYRA1000119 | 0 | 3.456 | 0 | 0 | 1.631 | 2.594 | 0 |
| HSYRA1000137 | 0 | 0 | 0 | 0 | 0 | 21.029 | 0 |
| HSYRA1000152 | 0 | 0 | 0 | 0 | 4.230 | 0 | 0 |
| HSYRA1000178 | 0 | 0 | 0 | 4.462 | 0 | 14.149 | 0 |
| HSYRA2000137 | 0 | 0 | 0 | 8.068 | 0 | 0 | 0 |
| HSYRA2000159 | 0 | 0.906 | 0.812 | 0.858 | 0 | 1.361 | 0 |
| HSYRA2000224 | 0 | 0 | 0 | 3.200 | 0 | 0 | 0 |
| HSYRA2000232 | 0 | 0 | 0 | 0 | 2.622 | 0 | 0 |
| HSYRA2000248 | 0 | 0 | 0 | 2.419 | 2.410 | 0 | 0 |
| HSYRA2000605 | 0 | 0 | 0 | 10.648 | 0 | 33.760 | 0 |
| HSYRA2000640 | 0 | 0 | 0 | 3.032 | 0 | 0 | 0 |
| HSYRA2000743 | 0 | 0 | 0 | 2.755 | 0 | 0 | 0 |
| HSYRA2000787 | 0 | 0 | 0 | 0 | 0 | 17.550 | 0 |
| HSYRA2000828 | 0 | 0 | 19.193 | 0 | 0 | 8.039 | 0 |
| HSYRA2001105 | 0 | 20.710 | 0 | 4.903 | 0 | 0 | 0 |
| HSYRA2001138 | 0 | 0 | 1.717 | 0 | 0 | 1.439 | 0 |
| HSYRA2001225 | 0 | 5.557 | 0 | 0 | 0 | 0 | 0 |
| HSYRA2001396 | 0 | 0 | 0 | 2.704 | 5.388 | 0 | 0 |
| HSYRA2001476 | 0 | 0 | 3.252 | 1.718 | 3.424 | 0 | 0 |
| HSYRA2001574 | 0 | 0 | 0 | 0 | 0 | 4.246 | 0 |
| HSYRA2001615 | 0 | 0 | 0 | 0 | 7.355 | 0 | 0 |
| HSYRA2001621 | 0 | 0 | 4.944 | 0 | 0 | 0 | 0 |
| HSYRA2001638 | 0 | 17.000 | 15.235 | 4.025 | 0 | 0 | 0 |
| HSYRA2002741 | 0 | 0 | 47.403 | 0 | 0 | 0 | 0 |
| HSYRA2007650 | 0 | 0 | 0 | 8.465 | 0 | 0 | 0 |
| IMR321000210 | 0 | 0 | 0 | 0 | 0 | 5.719 | 0 |
| IMR321000219 | 0 | 0 | 1.891 | 0 | 0 | 4.751 | 0 |
| IMR321000266 | 0 | 21.271 | 0 | 5.036 | 0 | 0 | 0 |
| IMR322000121 | 0 | 0 | 7.509 | 0 | 0 | 0 | 0 |
| IMR322000127 | 0 | 0 | 10.435 | 0 | 0 | 0 | 0 |
| IMR322000144 | 0 | 12.260 | 0 | 0 | 0 | 0 | 0 |
| IMR322000178 | 0 | 0 | 0 | 0 | 6.596 | 0 | 0 |
| IMR322000223 | 0 | 0 | 0 | 3.635 | 0 | 0 | 0 |
| IMR322000243 | 0 | 0 | 5.779 | 1.527 | 3.043 | 0 | 0 |
| IMR322000316 | 0 | 0 | 0 | 0 | 1.472 | 0 | 0 |
| IMR322000730 | 0 | 0 | 0.923 | 0.488 | 0 | 0 | 2.982 |
| IMR322000775 | 0 | 0 | 0 | 0 | 14.704 | 0 | 0 |
| IMR322000811 | 0 | 0 | 0 | 0 | 50.126 | 0 | 0 |
| IMR322000918 | 0 | 0 | 0 | 0 | 10.395 | 0 | 0 |
| IMR322000935 | 0 | 11.254 | 10.085 | 0 | 5.310 | 0 | 0 |
| IMR322001049 | 0 | 0 | 0 | 10.309 | 20.543 | 0 | 0 |
| IMR322001218 | 0 | 0 | 7.541 | 0 | 0 | 0 | 0 |
| IMR322001435 | 0 | 0 | 0 | 1.672 | 3.332 | 10.602 | 0 |
| IMR322001541 | 0 | 0 | 0 | 3.011 | 0 | 0 | 0 |
| IMR322001584 | 0 | 9.383 | 0 | 2.222 | 0 | 0 | 0 |
| IMR322001600 | 0 | 3.631 | 2.169 | 0.287 | 0.571 | 5.451 | 0 |
| IMR322019070 | 0 | 0 | 18.022 | 0 | 0 | 0 | 0 |
| KIDNE1000145 | 0 | 0 | 0 | 4.922 | 0 | 0 | 0 |
| KIDNE2000085 | 0 | 0 | 6.927 | 0 | 0 | 0 | 0 |
| KIDNE2000252 | 0 | 0 | 0 | 3.042 | 3.031 | 0 | 0 |
| KIDNE2000383 | 0 | 0 | 0 | 8.765 | 17.465 | 0 | 0 |
| KIDNE2000403 | 0 | 0 | 0 | 1.470 | 0 | 0 | 0 |
| KIDNE2000497 | 0 | 0 | 21.122 | 0 | 0 | 0 | 0 |
| KIDNE2000517 | 0 | 0 | 0 | 0 | 5.554 | 0 | 0 |
| KIDNE2000574 | 0 | 0 | 5.042 | 2.664 | 0 | 0 | 16.287 |
| KIDNE2000909 | 0 | 0 | 0 | 3.654 | 7.281 | 0 | 0 |
| KIDNE2001162 | 0 | 0 | 0 | 0.831 | 0 | 0 | 10.155 |
| KIDNE2001467 | 0 | 1.959 | 0 | 1.855 | 0.924 | 0 | 0 |
| KIDNE2001636 | 0 | 0 | 0 | 0 | 33.516 | 0 | 0 |
| KIDNE2002883 | 0 | 0 | 15.278 | 4.036 | 0 | 0 | 0 |
| KIDNE2002980 | 0 | 0 | 21.230 | 0 | 0 | 0 | 0 |
| KIDNE2003305 | 0 | 6.326 | 0 | 0 | 2.985 | 0 | 18.313 |
| KIDNE2004411 | 0 | 0 | 0 | 15.166 | 0 | 0 | 0 |
| KIDNE2004475 | 0 | 0 | 0 | 0 | 9.129 | 0 | 0 |
| KIDNE2004626 | 0 | 0 | 0 | 22.938 | 0 | 0 | 0 |
| KIDNE2005676 | 0 | 0 | 0 | 2.504 | 0 | 0 | 0 |
| KIDNE2006009 | 0 | 27.712 | 0 | 0 | 0 | 0 | 0 |
| KIDNE2006248 | 0 | 0 | 0 | 0 | 0 | 1.754 | 0 |
| KIDNE2007328 | 0 | 6.650 | 0 | 3.149 | 0 | 0 | 0 |
| KIDNE2007352 | 0 | 15.623 | 0 | 3.699 | 0 | 0 | 0 |
| KIDNE2007356 | 0 | 0 | 0 | 0 | 50.427 | 0 | 0 |
| KIDNE2007954 | 0 | 0 | 0 | 3.752 | 0 | 0 | 0 |
| KIDNE2008048 | 0 | 0 | 0 | 2.864 | 0 | 0 | 11.672 |
| KIDNE2008116 | 0 | 0 | 0 | 0 | 0 | 91.653 | 0 |
| KIDNE2008403 | 0 | 0 | 0 | 2.420 | 0 | 0 | 0 |
| KIDNE2010430 | 0 | 0 | 0 | 16.971 | 0 | 0 | 0 |
| KIDNE2012440 | 0 | 0 | 0 | 0 | 0 | 9.988 | 0 |
| KIDNE2013845 | 0 | 0 | 7.529 | 1.989 | 0 | 0 | 0 |
| KIDNE2014290 | 0 | 0 | 0 | 11.229 | 0 | 0 | 0 |
| KIDNE2014325 | 0 | 28.434 | 0 | 0 | 0 | 0 | 0 |
| KIDNE2015445 | 0 | 0 | 0 | 6.114 | 0 | 0 | 0 |
| KIDNE2016371 | 0 | 0 | 0 | 16.034 | 0 | 0 | 0 |
| KIDNE2017040 | 0 | 0 | 0 | 0 | 0 | 18.974 | 0 |
| KIDNE2018493 | 0 | 0 | 0 | 0 | 25.273 | 0 | 0 |
| LIVER1000079 | 0 | 13.171 | 0 | 0 | 0 | 0 | 0 |
| LIVER1000132 | 0 | 0 | 0 | 0 | 1.775 | 0 | 10.888 |
| LIVER2000416 | 0 | 0 | 0 | 0 | 0 | 12.767 | 49.230 |
| LIVER2000446 | 0 | 0 | 0 | 7.321 | 0 | 0 | 0 |
| LIVER2001539 | 0 | 0 | 0 | 0 | 0 | 28.099 | 0 |
| LIVER2001608 | 0 | 0 | 0 | 0 | 9.315 | 7.410 | 0 |
| LIVER2002842 | 0 | 0 | 0 | 3.397 | 0 | 0 | 0 |
| LIVER2005028 | 0 | 0 | 0 | 0 | 26.795 | 0 | 0 |
| LIVER2005789 | 0 | 0 | 0 | 1.602 | 0 | 0 | 19.587 |
| LIVER2005981 | 0 | 0 | 0 | 0 | 10.154 | 0 | 0 |
| LIVER2007783 | 0 | 0 | 13.821 | 0 | 0 | 0 | 0 |
| MAMGL1000032 | 0 | 0 | 0 | 0.287 | 0 | 0 | 0 |
| MAMGL1000035 | 0 | 0 | 0 | 0 | 0 | 1.136 | 0 |
| MAMGL1000096 | 0 | 2.265 | 0 | 0 | 0 | 0 | 0 |
| MAMGL1000173 | 0 | 0 | 0 | 0 | 1.880 | 0 | 0 |
| MESAN1000101 | 0 | 0 | 0 | 20.545 | 0 | 0 | 0 |
| MESAN1000126 | 0 | 0 | 0 | 9.534 | 0 | 0 | 0 |
| MESAN2000264 | 0 | 14.812 | 0 | 0 | 0 | 0 | 0 |
| MESAN2000267 | 0 | 0 | 0 | 2.915 | 0 | 0 | 0 |
| MESAN2000457 | 0 | 0 | 0 | 63.433 | 0 | 0 | 0 |
| MESAN2000572 | 0 | 0 | 0 | 1.820 | 0 | 0 | 0 |
| MESAN2000608 | 0 | 5.911 | 0 | 0 | 8.366 | 0 | 0 |
| MESAN2000620 | 0 | 0 | 0 | 32.430 | 0 | 0 | 0 |
| MESAN2001154 | 0 | 10.015 | 0 | 2.371 | 0 | 0 | 0 |
| MESAN2001450 | 0 | 0 | 0 | 0 | 0 | 16.273 | 0 |
| MESAN2002086 | 0 | 0 | 2.804 | 2.223 | 1.476 | 7.047 | 0 |
| MESAN2002724 | 0 | 0 | 0 | 4.924 | 0 | 0 | 0 |
| MESAN2002790 | 0 | 0 | 0 | 13.058 | 0 | 0 | 0 |
| MESAN2002940 | 0 | 0 | 0 | 0 | 0 | 17.945 | 0 |
| MESAN2003035 | 0 | 0 | 0 | 12.755 | 0 | 0 | 0 |
| MESAN2003058 | 0 | 0 | 0 | 0 | 0 | 33.793 | 0 |
| MESAN2003444 | 0 | 0 | 0 | 0 | 13.211 | 0 | 0 |
| MESAN2003622 | 0 | 0 | 0 | 1.070 | 0 | 0 | 0 |
| MESAN2005303 | 0 | 0 | 0 | 14.099 | 0 | 0 | 0 |
| MESAN2005766 | 0 | 0 | 0 | 0 | 3.497 | 0 | 0 |
| MESAN2005957 | 0 | 0 | 0 | 17.574 | 0 | 0 | 0 |
| MESAN2005958 | 0 | 0 | 0 | 9.585 | 0 | 0 | 0 |
| MESAN2006563 | 0 | 0 | 0 | 0 | 2.449 | 0 | 0 |
| MESAN2006743 | 0 | 0 | 0 | 0 | 0 | 21.750 | 0 |
| MESAN2008415 | 0 | 0 | 0 | 0 | 0 | 60.344 | 0 |
| MESAN2008936 | 0 | 0 | 0 | 0 | 0 | 22.448 | 0 |
| MESAN2009580 | 0 | 0 | 0 | 0 | 0 | 6.266 | 0 |
| MESAN2011627 | 0 | 28.288 | 0 | 0 | 0 | 0 | 0 |
| MESAN2011984 | 0 | 12.823 | 0 | 3.036 | 6.050 | 0 | 0 |
| MESAN2012054 | 0 | 0 | 0 | 4.540 | 0 | 0 | 0 |
| MESAN2012113 | 0 | 0 | 0 | 0 | 0 | 33.291 | 0 |
| MESAN2018699 | 0 | 0 | 0 | 27.399 | 0 | 0 | 0 |
| NB9N41000011 | 0 | 0 | 0 | 3.788 | 0 | 0 | 0 |
| NB9N42000342 | 0 | 0 | 0 | 0 | 0 | 4.068 | 0 |
| NHNPC2000187 | 0 | 0 | 3.048 | 2.416 | 0 | 0 | 0 |
| NHNPC2000206 | 0 | 0 | 0 | 2.444 | 0 | 0 | 0 |
| NHNPC2001931 | 0 | 0 | 9.644 | 1.699 | 0 | 0 | 10.384 |
| NOVAR1000091 | 0 | 0 | 0 | 3.446 | 0 | 0 | 0 |
| NOVAR2000038 | 0 | 0 | 0 | 0 | 2.203 | 0 | 0 |
| NT2NE1000163 | 0 | 0 | 2.728 | 0.721 | 0 | 0 | 0 |
| NT2NE2000056 | 0 | 0 | 0 | 0.699 | 0 | 0 | 0 |
| NT2NE2000174 | 0 | 0 | 0 | 0.679 | 0 | 2.153 | 0 |
| NT2NE2000195 | 0 | 11.554 | 0 | 0 | 5.451 | 0 | 0 |
| NT2NE2000259 | 0 | 21.143 | 0 | 0 | 0 | 0 | 0 |
| NT2NE2000260 | 0 | 5.448 | 0.814 | 0.430 | 0 | 0 | 0 |
| NT2NE2000327 | 0 | 0 | 0 | 4.477 | 0 | 0 | 0 |
| NT2NE2000383 | 0 | 11.422 | 0 | 2.704 | 0 | 0 | 0 |
| NT2NE2000384 | 0 | 0 | 0 | 4.096 | 0 | 12.987 | 0 |
| NT2NE2000575 | 0 | 0 | 3.239 | 0.428 | 0.853 | 1.356 | 0 |
| NT2NE2000707 | 0 | 0 | 0 | 0 | 1.187 | 1.888 | 0 |
| NT2NE2000963 | 0 | 0 | 1.545 | 0.408 | 0 | 1.294 | 9.982 |
| NT2NE2001000 | 0 | 0 | 5.797 | 0 | 3.052 | 0 | 0 |
| NT2NE2001337 | 0 | 0 | 0 | 8.087 | 0 | 25.641 | 0 |
| NT2NE2001435 | 0 | 7.991 | 0 | 0 | 3.770 | 0 | 0 |
| NT2NE2001530 | 0 | 0 | 0 | 12.452 | 0 | 0 | 0 |
| NT2NE2001545 | 0 | 13.468 | 0 | 3.189 | 6.354 | 0 | 0 |
| NT2NE2001655 | 0 | 0 | 0 | 2.492 | 4.965 | 0 | 0 |
| NT2NE2001793 | 0 | 0 | 0 | 3.692 | 0 | 0 | 0 |
| NT2NE2002870 | 0 | 0 | 0 | 0 | 0 | 12.880 | 0 |
| NT2NE2004716 | 0 | 0 | 0 | 0 | 0 | 0 | 41.761 |
| NT2NE2005371 | 0 | 0 | 0 | 0 | 19.554 | 0 | 0 |
| NT2NE2005441 | 0 | 0 | 0 | 0 | 0 | 0 | 67.598 |
| NT2NE2005688 | 0 | 0 | 5.030 | 1.329 | 0 | 4.213 | 0 |
| NT2NE2005876 | 0 | 11.088 | 0 | 0 | 0 | 0 | 0 |
| NT2NE2006458 | 0 | 0 | 0 | 0 | 0 | 7.976 | 0 |
| NT2NE2006478 | 0 | 0 | 0 | 19.122 | 0 | 0 | 0 |
| NT2NE2010842 | 0 | 14.174 | 0 | 0 | 0 | 10.641 | 0 |
| NT2NE2011119 | 0 | 34.804 | 0 | 0 | 0 | 0 | 0 |
| NT2NE2012928 | 0 | 0 | 0 | 0 | 30.876 | 0 | 0 |
| NT2NE2014104 | 0 | 0 | 0 | 2.987 | 0 | 0 | 0 |
| NT2RI1000027 | 0 | 0 | 0 | 1.799 | 0 | 0 | 0 |
| NT2RI2000064 | 0 | 0 | 7.251 | 0.958 | 0 | 6.074 | 11.711 |
| NT2RI2000107 | 0 | 52.232 | 0 | 0 | 0 | 0 | 0 |
| NT2RI2000255 | 0 | 0 | 0 | 2.130 | 8.490 | 0 | 0 |
| NT2RI2000282 | 0 | 0 | 0 | 0 | 1.122 | 1.786 | 0 |
| NT2RI2000671 | 0 | 0 | 0 | 0 | 3.730 | 0 | 0 |
| NT2RI2000685 | 0 | 0 | 6.674 | 0 | 3.514 | 0 | 0 |
| NT2RI2000689 | 0 | 0 | 0 | 0 | 10.967 | 0 | 0 |
| NT2RI2000727 | 0 | 0 | 0 | 0 | 0 | 1.460 | 0 |
| NT2RI2000822 | 0 | 0 | 71.758 | 0 | 0 | 0 | 0 |
| NT2RI2000974 | 0 | 0 | 14.530 | 0 | 0 | 0 | 0 |
| NT2RI2001230 | 0 | 5.798 | 0 | 1.373 | 0 | 4.352 | 0 |
| NT2RI2001342 | 0 | 0 | 0 | 2.809 | 0 | 0 | 0 |
| NT2RI2001540 | 0 | 2.139 | 1.917 | 1.013 | 0.807 | 0.642 | 3.716 |
| NT2RI2001595 | 0 | 0 | 0 | 0 | 28.159 | 0 | 0 |
| NT2RI2001657 | 0 | 0 | 0 | 1.904 | 1.897 | 3.019 | 0 |
| NT2RI2001859 | 0 | 7.294 | 6.537 | 0 | 3.441 | 0 | 0 |
| NT2RI2002091 | 0 | 0.202 | 0.361 | 1.552 | 0.618 | 1.665 | 1.751 |
| NT2RI2002316 | 0 | 0 | 5.401 | 0 | 0 | 0 | 0 |
| NT2RI2002359 | 0 | 61.860 | 0 | 0 | 0 | 0 | 0 |
| NT2RI2002391 | 0 | 0 | 0 | 1.443 | 0 | 0 | 0 |
| NT2RI2002530 | 0 | 0 | 0 | 0 | 0 | 34.059 | 0 |
| NT2RI2002554 | 0 | 0 | 0 | 3.227 | 0 | 0 | 0 |
| NT2RI2002654 | 0 | 4.416 | 0 | 1.045 | 4.167 | 0 | 0 |
| NT2RI2002699 | 0 | 0 | 0 | 20.348 | 0 | 0 | 0 |
| NT2RI2002729 | 0 | 15.809 | 0 | 0 | 0 | 0 | 0 |
| NT2RI2002819 | 0 | 27.984 | 0 | 0 | 0 | 0 | 0 |
| NT2RI2002847 | 0 | 9.435 | 0 | 0 | 4.452 | 0 | 0 |
| NT2RI2002865 | 0 | 0 | 0 | 12.068 | 0 | 0 | 0 |
| NT2RI2002958 | 0 | 0 | 0 | 0 | 13.657 | 0 | 0 |
| NT2RI2003019 | 0 | 0 | 0 | 0 | 18.547 | 0 | 0 |
| NT2RI2003051 | 0 | 0 | 0 | 10.932 | 0 | 0 | 0 |
| NT2RI2003304 | 0 | 1.973 | 0 | 0.467 | 0 | 0 | 0 |
| NT2RI2003317 | 0 | 0 | 0 | 0 | 5.590 | 0 | 0 |
| NT2RI2003338 | 0 | 0 | 6.821 | 0 | 0 | 5.713 | 0 |
| NT2RI2003407 | 0 | 0 | 0 | 4.308 | 0 | 0 | 0 |
| NT2RI2003420 | 0 | 0 | 0 | 2.741 | 0 | 0 | 0 |
| NT2RI2003556 | 0 | 2.860 | 0 | 0 | 1.349 | 0 | 0 |
| NT2RI2003695 | 0 | 0 | 0 | 1.401 | 5.585 | 0 | 0 |
| NT2RI2003738 | 0 | 0 | 0 | 0 | 3.102 | 0 | 0 |
| NT2RI2003921 | 0 | 0 | 0 | 0 | 1.682 | 2.676 | 0 |
| NT2RI2003993 | 0 | 0 | 0 | 6.756 | 0 | 0 | 0 |
| NT2RI2004230 | 0 | 0 | 0 | 0 | 4.069 | 6.474 | 0 |
| NT2RI2004398 | 0 | 0 | 0 | 8.866 | 0 | 0 | 0 |
| NT2RI2004608 | 0 | 0 | 0 | 6.584 | 0 | 0 | 0 |
| NT2RI2005061 | 0 | 32.789 | 0 | 7.763 | 0 | 0 | 0 |
| NT2RI2005115 | 0 | 0 | 0 | 0 | 0 | 34.481 | 0 |
| NT2RI2005116 | 0 | 9.608 | 0 | 0 | 4.533 | 0 | 0 |
| NT2RI2005335 | 0 | 2.910 | 0 | 1.378 | 1.373 | 0 | 0 |
| NT2RI2005353 | 0 | 0 | 0 | 4.100 | 0 | 0 | 0 |
| NT2RI2005405 | 0 | 2.110 | 0 | 0 | 0.996 | 6.336 | 0 |
| NT2RI2005723 | 0 | 0 | 3.380 | 0 | 1.780 | 0 | 0 |
| NT2RI2005811 | 0 | 11.516 | 0 | 0 | 5.433 | 0 | 0 |
| NT2RI2005814 | 0 | 0 | 0 | 0 | 0 | 7.349 | 0 |
| NT2RI2005966 | 0 | 0 | 0 | 0 | 66.119 | 0 | 0 |
| NT2RI2006070 | 0 | 0 | 0 | 0 | 7.288 | 11.597 | 0 |
| NT2RI2006553 | 0 | 0 | 0 | 0 | 13.117 | 0 | 0 |
| NT2RI2006565 | 0 | 0 | 0 | 10.634 | 0 | 0 | 0 |
| NT2RI2006647 | 0 | 0 | 0 | 8.161 | 0 | 0 | 0 |
| NT2RI2006682 | 0 | 0 | 16.576 | 0 | 0 | 13.885 | 0 |
| NT2RI2006686 | 0 | 0 | 78.754 | 0 | 0 | 0 | 0 |
| NT2RI2006825 | 0 | 0 | 0 | 9.758 | 0 | 0 | 0 |
| NT2RI2006855 | 0 | 0 | 58.068 | 0 | 0 | 0 | 0 |
| NT2RI2006856 | 0 | 0 | 0 | 0.834 | 1.661 | 0 | 10.191 |
| NT2RI2007054 | 0 | 0 | 0 | 0 | 4.604 | 0 | 0 |
| NT2RI2007133 | 0 | 0 | 0 | 0 | 1.391 | 0 | 0 |
| NT2RI2007377 | 0 | 0 | 0 | 2.084 | 0 | 0 | 0 |
| NT2RI2007384 | 0 | 0 | 2.917 | 1.541 | 0 | 4.887 | 0 |
| NT2RI2007498 | 0 | 0 | 0 | 3.629 | 0 | 0 | 0 |
| NT2RI2007589 | 0 | 0 | 7.194 | 0 | 0 | 0 | 0 |
| NT2RI2007853 | 0 | 0 | 0 | 0 | 0 | 25.267 | 0 |
| NT2RI2007879 | 0 | 0 | 0 | 0 | 5.629 | 0 | 0 |
| NT2RI2007987 | 0 | 0 | 12.129 | 0 | 0 | 0 | 0 |
| NT2RI2008141 | 0 | 0 | 0 | 7.611 | 0 | 0 | 0 |
| NT2RI2008221 | 0 | 8.333 | 0 | 0 | 0 | 0 | 0 |
| NT2RI2008336 | 0 | 0 | 0 | 0 | 4.262 | 0 | 0 |
| NT2RI2008396 | 0 | 0 | 0 | 1.569 | 1.563 | 0 | 0 |
| NT2RI2008526 | 0 | 0 | 0 | 0 | 0 | 8.652 | 0 |
| NT2RI2008749 | 0 | 0 | 7.617 | 2.012 | 0 | 0 | 0 |
| NT2RI2008791 | 0 | 0 | 0 | 0 | 4.138 | 6.583 | 0 |
| NT2RI2008812 | 0 | 0 | 0 | 0 | 0 | 40.851 | 0 |
| NT2RI2009037 | 0 | 6.337 | 0 | 0 | 0 | 4.757 | 0 |
| NT2RI2009065 | 0 | 3.511 | 0 | 0 | 1.656 | 0 | 0 |
| NT2RI2009239 | 0 | 0 | 0 | 2.488 | 0 | 0 | 0 |
| NT2RI2009269 | 0 | 0 | 0 | 3.517 | 0 | 5.576 | 0 |
| NT2RI2009281 | 0 | 0 | 0 | 0 | 4.991 | 0 | 0 |
| NT2RI2009583 | 0 | 0 | 4.521 | 0.398 | 1.587 | 0 | 0 |
| NT2RI2012350 | 0 | 0 | 0 | 0.712 | 0 | 2.258 | 0 |
| NT2RI2012990 | 0 | 0 | 0 | 0 | 0 | 75.640 | 0 |
| NT2RI2013345 | 0 | 0 | 63.707 | 0 | 0 | 0 | 0 |
| NT2RI2014247 | 0 | 0 | 0 | 0 | 54.091 | 0 | 0 |
| NT2RI2015342 | 0 | 0 | 0 | 2.677 | 0 | 0 | 0 |
| NT2RI2015533 | 0 | 0 | 10.469 | 0 | 0 | 0 | 0 |
| NT2RI3006284 | 0 | 0 | 0 | 3.682 | 0 | 0 | 0 |
| NT2RI3006340 | 0 | 0 | 55.006 | 0 | 0 | 0 | 0 |
| NT2RI3006666 | 0 | 0 | 0 | 37.157 | 0 | 0 | 0 |
| NT2RI3007543 | 0 | 0 | 5.715 | 1.510 | 0 | 0 | 0 |
| NT2RP6000005 | 0 | 16.533 | 0 | 0 | 0 | 0 | 0 |
| NT2RP6000032 | 0 | 58.947 | 0 | 0 | 0 | 0 | 0 |
| NT2RP6000039 | 0 | 0 | 0 | 12.765 | 0 | 0 | 0 |
| NT2RP6000072 | 0 | 0 | 10.884 | 0 | 0 | 0 | 0 |
| NT2RP6000077 | 0 | 0 | 0 | 22.879 | 0 | 0 | 0 |
| NT2RP6000078 | 0 | 5.405 | 0 | 0 | 0 | 0 | 0 |
| NT2RP6000110 | 0 | 0 | 0 | 4.873 | 0 | 0 | 0 |
| NT2RP6000123 | 0 | 0 | 0 | 0.697 | 5.553 | 2.209 | 0 |
| NT2RP7000069 | 0 | 0 | 0 | 11.990 | 0 | 0 | 0 |
| NT2RP7000076 | 0 | 9.162 | 0 | 0 | 8.645 | 0 | 0 |
| NT2RP7000173 | 0 | 0 | 0 | 0.989 | 1.972 | 0 | 0 |
| NT2RP7000271 | 0 | 0 | 0 | 5.398 | 0 | 0 | 0 |
| NT2RP7000477 | 0 | 0 | 13.327 | 0 | 0 | 0 | 0 |
| NT2RP7000600 | 0 | 0 | 0 | 3.944 | 0 | 0 | 0 |
| NT2RP7001166 | 0 | 0 | 0 | 1.331 | 0 | 0 | 0 |
| NT2RP7001283 | 0 | 0 | 0 | 0 | 1.996 | 0 | 0 |
| NT2RP7001962 | 0 | 0 | 0 | 9.455 | 0 | 0 | 0 |
| NT2RP7002028 | 0 | 0 | 0 | 0 | 0 | 11.339 | 0 |
| NT2RP7002379 | 0 | 0 | 0 | 0 | 1.785 | 2.840 | 0 |
| NT2RP7002619 | 0 | 8.190 | 0 | 1.939 | 0 | 6.148 | 0 |
| NT2RP7002982 | 0 | 0 | 0 | 0 | 55.144 | 0 | 0 |
| NT2RP7003203 | 0 | 13.884 | 0 | 3.287 | 0 | 0 | 0 |
| NT2RP7003261 | 0 | 0 | 0 | 2.418 | 0 | 0 | 0 |
| NT2RP7003319 | 0 | 0 | 6.146 | 3.247 | 0 | 0 | 0 |
| NT2RP7003629 | 0 | 0 | 0 | 20.137 | 0 | 0 | 0 |
| NT2RP7003647 | 0 | 0 | 0 | 6.283 | 0 | 0 | 0 |
| NT2RP7003688 | 0 | 0 | 0 | 2.628 | 2.619 | 0 | 0 |
| NT2RP7003724 | 0 | 0 | 0 | 16.434 | 0 | 0 | 0 |
| NT2RP7004114 | 0 | 0 | 0 | 9.924 | 0 | 0 | 0 |
| NT2RP7004173 | 0 | 0 | 0 | 4.077 | 0 | 0 | 0 |
| NT2RP7004196 | 0 | 0 | 0 | 0 | 0 | 7.393 | 0 |
| NT2RP7004373 | 0 | 0 | 0 | 6.284 | 4.174 | 0 | 0 |
| NT2RP7004728 | 0 | 11.814 | 0 | 2.797 | 0 | 0 | 0 |
| NT2RP7004751 | 0 | 0 | 0 | 0 | 0 | 2.641 | 0 |
| NT2RP7004925 | 0 | 0 | 0 | 2.247 | 4.478 | 0 | 0 |
| NT2RP7004946 | 0 | 6.471 | 0 | 3.064 | 0 | 0 | 0 |
| NT2RP7005513 | 0 | 0 | 11.385 | 0 | 0 | 9.537 | 0 |
| NT2RP7005675 | 0 | 0 | 0 | 5.176 | 0 | 0 | 0 |
| NT2RP7005750 | 0 | 0 | 0 | 0 | 25.379 | 0 | 0 |
| NT2RP7006033 | 0 | 0 | 0 | 13.681 | 0 | 0 | 0 |
| NT2RP7006075 | 0 | 0 | 0 | 9.326 | 0 | 0 | 0 |
| NT2RP7006141 | 0 | 0 | 0 | 0 | 2.619 | 0 | 0 |
| NT2RP7006188 | 0 | 0 | 0 | 9.266 | 0 | 0 | 0 |
| NT2RP7006619 | 0 | 0 | 0 | 3.928 | 0 | 0 | 0 |
| NT2RP7006980 | 0 | 0 | 0 | 30.014 | 0 | 0 | 0 |
| NT2RP7007154 | 0 | 0 | 0 | 1.015 | 0 | 0 | 0 |
| NT2RP7007177 | 0 | 0 | 0 | 0 | 0 | 7.684 | 0 |
| NT2RP7007387 | 0 | 0 | 0 | 3.653 | 0 | 0 | 0 |
| NT2RP7007537 | 0 | 0 | 8.283 | 0 | 4.361 | 0 | 0 |
| NT2RP7007975 | 0 | 0 | 0 | 2.429 | 0 | 0 | 0 |
| NT2RP7008161 | 0 | 0 | 0 | 11.173 | 0 | 0 | 0 |
| NT2RP7008167 | 0 | 0 | 0 | 0 | 3.219 | 0 | 0 |
| NT2RP7008190 | 0 | 0 | 0 | 2.361 | 4.704 | 0 | 0 |
| NT2RP7008396 | 0 | 0 | 0 | 21.908 | 0 | 0 | 0 |
| NT2RP7008543 | 0 | 0 | 0 | 9.792 | 9.756 | 0 | 0 |
| NT2RP7009097 | 0 | 0 | 0 | 3.200 | 0 | 0 | 0 |
| NT2RP7009147 | 0 | 0 | 0 | 2.835 | 0 | 0 | 0 |
| NT2RP7009149 | 0 | 0 | 8.058 | 0 | 0 | 0 | 0 |
| NT2RP7009259 | 0 | 0 | 0 | 4.000 | 0 | 0 | 0 |
| NT2RP7009394 | 0 | 0 | 0 | 2.152 | 0 | 0 | 0 |
| NT2RP7009482 | 0 | 0 | 0 | 0 | 1.644 | 2.615 | 0 |
| NT2RP7009498 | 0 | 0 | 0 | 3.587 | 0 | 0 | 0 |
| NT2RP7014910 | 0 | 0 | 0 | 2.128 | 0 | 0 | 0 |
| NT2RP7015512 | 0 | 0 | 0 | 34.509 | 0 | 0 | 0 |
| NT2RP7019064 | 0 | 0 | 0 | 45.711 | 0 | 0 | 0 |
| NT2RP8000435 | 0 | 0 | 0 | 23.746 | 0 | 0 | 0 |
| NT2RP8007920 | 0 | 14.869 | 0 | 0 | 0 | 0 | 0 |
| NTONG1000033 | 0 | 0 | 0 | 2.180 | 0 | 0 | 0 |
| NTONG1000052 | 0 | 0 | 35.389 | 0 | 5.081 | 0 | 0 |
| NTONG1000098 | 0 | 0 | 0 | 1.134 | 0 | 3.595 | 0 |
| NTONG1000123 | 0 | 0 | 31.366 | 0 | 0 | 0 | 0 |
| NTONG1000182 | 0 | 0 | 25.018 | 2.203 | 4.390 | 0 | 0 |
| NTONG1000257 | 0 | 0 | 0 | 0 | 0 | 6.210 | 0 |
| NTONG1000264 | 0 | 7.326 | 0 | 0 | 0 | 5.500 | 0 |
| NTONG2000265 | 0 | 0 | 0 | 1.517 | 0 | 0 | 0 |
| NTONG2000334 | 0 | 0 | 0 | 0 | 7.251 | 0 | 0 |
| NTONG2000858 | 0 | 0 | 58.364 | 0 | 0 | 0 | 0 |
| NTONG2000876 | 0 | 10.466 | 0 | 4.956 | 4.938 | 7.857 | 0 |
| NTONG2000878 | 0 | 0 | 0 | 2.102 | 0 | 0 | 25.694 |
| NTONG2000977 | 0 | 0 | 0 | 0 | 0 | 5.547 | 0 |
| NTONG2000985 | 0 | 0 | 12.558 | 0 | 0 | 0 | 0 |
| NTONG2001220 | 0 | 0 | 0 | 0 | 20.407 | 0 | 0 |
| NTONG2001550 | 0 | 0 | 0 | 0 | 12.343 | 0 | 0 |
| NTONG2001567 | 0 | 0 | 0 | 0 | 3.262 | 1.730 | 20.015 |
| NTONG2001612 | 0 | 0 | 0 | 0 | 0 | 6.334 | 0 |
| NTONG2002948 | 0 | 0 | 14.957 | 0 | 7.874 | 0 | 0 |
| NTONG2003454 | 0 | 0 | 0 | 10.611 | 0 | 0 | 0 |
| NTONG2003852 | 0 | 50.493 | 0 | 0 | 0 | 0 | 0 |
| NTONG2004614 | 0 | 0 | 0 | 10.539 | 0 | 0 | 0 |
| NTONG2004844 | 0 | 0 | 0 | 0 | 0 | 9.144 | 0 |
| NTONG2005391 | 0 | 0 | 0 | 12.447 | 0 | 0 | 0 |
| NTONG2005497 | 0 | 0 | 0 | 0 | 0 | 1.546 | 0 |
| NTONG2005520 | 0 | 0 | 0 | 1.185 | 0 | 3.756 | 0 |
| NTONG2005801 | 0 | 0 | 0 | 9.512 | 0 | 0 | 0 |
| NTONG2006484 | 0 | 0 | 0 | 0 | 30.659 | 0 | 0 |
| NTONG2006783 | 0 | 0 | 76.282 | 0 | 0 | 0 | 0 |
| NTONG2007034 | 0 | 0 | 47.753 | 0 | 0 | 0 | 0 |
| NTONG2007658 | 0 | 15.042 | 0 | 0 | 0 | 0 | 0 |
| NTONG2007756 | 0 | 0 | 45.466 | 0 | 0 | 0 | 0 |
| NTONG2008483 | 0 | 0 | 5.382 | 2.844 | 0 | 4.508 | 0 |
| NTONG2008944 | 0 | 0 | 0 | 25.982 | 0 | 0 | 0 |
| NTONG2009060 | 0 | 0 | 18.770 | 0 | 0 | 0 | 0 |
| OCBBF1000146 | 0 | 0 | 0 | 0 | 3.059 | 0 | 0 |
| OCBBF1000188 | 0 | 0 | 0 | 3.706 | 0 | 0 | 0 |
| OCBBF1000315 | 0 | 0 | 0 | 15.571 | 0 | 0 | 0 |
| OCBBF2000013 | 0 | 0 | 0 | 0 | 0 | 10.980 | 0 |
| OCBBF2000015 | 0 | 0 | 0 | 8.448 | 0 | 0 | 0 |
| OCBBF2000824 | 0 | 0 | 0 | 0 | 21.310 | 0 | 0 |
| OCBBF2000846 | 0 | 0 | 0 | 1.302 | 0 | 0 | 0 |
| OCBBF2000986 | 0 | 0 | 0 | 3.430 | 0 | 0 | 0 |
| OCBBF2001210 | 0 | 0 | 0 | 6.453 | 0 | 0 | 0 |
| OCBBF2001307 | 0 | 0 | 0 | 2.960 | 0 | 0 | 0 |
| OCBBF2001492 | 0 | 5.157 | 4.622 | 3.663 | 2.433 | 3.871 | 0 |
| OCBBF2001586 | 0 | 0 | 0 | 0 | 12.716 | 0 | 0 |
| OCBBF2001639 | 0 | 0 | 0 | 0 | 16.676 | 0 | 0 |
| OCBBF2001687 | 0 | 0 | 0 | 3.423 | 0 | 0 | 0 |
| OCBBF2001983 | 0 | 0 | 0 | 64.284 | 0 | 0 | 0 |
| OCBBF2002086 | 0 | 0 | 0 | 11.467 | 0 | 0 | 0 |
| OCBBF2002626 | 0 | 0 | 0 | 0 | 2.995 | 0 | 0 |
| OCBBF2003518 | 0 | 0 | 0 | 1.835 | 0 | 0 | 0 |
| OCBBF2004273 | 0 | 0 | 0 | 1.595 | 0 | 0 | 0 |
| OCBBF2005956 | 0 | 21.338 | 0 | 0 | 0 | 0 | 0 |
| OCBBF2006005 | 0 | 35.834 | 0 | 8.484 | 0 | 0 | 0 |
| OCBBF2006148 | 0 | 0 | 0 | 0 | 0 | 38.212 | 0 |
| OCBBF2006849 | 0 | 0 | 0 | 0 | 7.167 | 0 | 0 |
| OCBBF2007354 | 0 | 0 | 0 | 1.788 | 0 | 0 | 0 |
| OCBBF2007622 | 0 | 0 | 0 | 0 | 0 | 0 | 79.879 |
| OCBBF2007829 | 0 | 0 | 0 | 1.954 | 0 | 0 | 0 |
| OCBBF2009095 | 0 | 0 | 0 | 0 | 0 | 37.084 | 0 |
| OCBBF2009603 | 0 | 0 | 0 | 58.892 | 0 | 0 | 0 |
| OCBBF2009772 | 0 | 0 | 7.932 | 0 | 0 | 0 | 0 |
| OCBBF2011887 | 0 | 0 | 0 | 8.370 | 0 | 0 | 0 |
| OCBBF2012095 | 0 | 0 | 0 | 27.233 | 0 | 0 | 0 |
| OCBBF2012714 | 0 | 43.630 | 0 | 0 | 0 | 0 | 0 |
| OCBBF2012821 | 0 | 0 | 0 | 4.453 | 0 | 0 | 0 |
| OCBBF2014576 | 0 | 0 | 0 | 6.503 | 0 | 0 | 0 |
| OCBBF2015503 | 0 | 0 | 0 | 13.355 | 0 | 0 | 0 |
| OCBBF2016928 | 0 | 0 | 0 | 4.874 | 0 | 0 | 0 |
| OCBBF2017035 | 0 | 0 | 8.166 | 0 | 0 | 0 | 0 |
| OCBBF2017055 | 0 | 0 | 0 | 31.933 | 0 | 0 | 0 |
| OCBBF2017398 | 0 | 9.669 | 0 | 0 | 0 | 0 | 0 |
| OCBBF2017458 | 0 | 0 | 0 | 0 | 0 | 31.413 | 0 |
| OCBBF2017754 | 0 | 0 | 0 | 6.682 | 0 | 0 | 0 |
| OCBBF2017791 | 0 | 0 | 0 | 11.397 | 0 | 0 | 0 |
| OCBBF2017888 | 0 | 0 | 0 | 30.321 | 0 | 0 | 0 |
| OCBBF2018012 | 0 | 0 | 0 | 9.990 | 0 | 0 | 0 |
| OCBBF2018873 | 0 | 0 | 0 | 0 | 4.914 | 0 | 0 |
| OCBBF2018956 | 0 | 0 | 0 | 0 | 0 | 19.099 | 0 |
| OCBBF2024850 | 0 | 0 | 0 | 7.910 | 0 | 0 | 0 |
| OCBBF2026368 | 0 | 0 | 0 | 14.706 | 0 | 0 | 0 |
| OCBBF3000323 | 0 | 0 | 0 | 5.919 | 11.795 | 0 | 0 |
| OCBBF3001333 | 0 | 0 | 0 | 5.718 | 0 | 0 | 0 |
| OCBBF3004487 | 0 | 0 | 0 | 8.063 | 0 | 0 | 0 |
| PANCR1000012 | 100 | 0 | 0 | 0 | 0 | 0 | 0 |
| PANCR1000021 | 100 | 0 | 0 | 0 | 0 | 0 | 0 |
| PANCR1000081 | 98.068 | 0 | 0 | 0 | 0 | 0 | 0 |
| PANCR1000086 | 100 | 0 | 0 | 0 | 0 | 0 | 0 |
| PANCR1000091 | 98.673 | 0 | 0 | 0 | 0 | 0 | 0 |
| PANCR1000185 | 100 | 0 | 0 | 0 | 0 | 0 | 0 |
| PEBLM1000024 | 0 | 100 | 0 | 0 | 0 | 0 | 0 |
| PEBLM1000029 | 0 | 100 | 0 | 0 | 0 | 0 | 0 |
| PEBLM1000034 | 0 | 52.261 | 0 | 0 | 0 | 0 | 0 |
| PEBLM1000062 | 0 | 100 | 0 | 0 | 0 | 0 | 0 |
| PEBLM1000066 | 0 | 35.622 | 0 | 0 | 16.807 | 0 | 0 |
| PEBLM1000068 | 0 | 100 | 0 | 0 | 0 | 0 | 0 |
| PEBLM1000071 | 0 | 100 | 0 | 0 | 0 | 0 | 0 |
| PEBLM1000083 | 0 | 100 | 0 | 0 | 0 | 0 | 0 |
| PEBLM1000144 | 0 | 45.080 | 0 | 0 | 0 | 0 | 0 |
| PEBLM1000147 | 0 | 100 | 0 | 0 | 0 | 0 | 0 |
| PEBLM1000174 | 0 | 35.947 | 0 | 8.511 | 0 | 0 | 0 |
| PEBLM1000180 | 0 | 100 | 0 | 0 | 0 | 0 | 0 |
| PEBLM2000030 | 0 | 27.664 | 0 | 0 | 0 | 0 | 0 |
| PEBLM2000112 | 0 | 100 | 0 | 0 | 0 | 0 | 0 |
| PEBLM2000126 | 0 | 81.206 | 0 | 0 | 0 | 0 | 0 |
| PEBLM2000147 | 0 | 19.178 | 0 | 0 | 0 | 0 | 0 |
| PEBLM2000170 | 0 | 100 | 0 | 0 | 0 | 0 | 0 |
| PEBLM2000180 | 0 | 100 | 0 | 0 | 0 | 0 | 0 |
| PEBLM2000213 | 0 | 100 | 0 | 0 | 0 | 0 | 0 |
| PEBLM2000222 | 0 | 8.732 | 0 | 0 | 0 | 0 | 0 |
| PEBLM2000248 | 0 | 100 | 0 | 0 | 0 | 0 | 0 |
| PEBLM2000267 | 0 | 10.569 | 0 | 0 | 0 | 0 | 0 |
| PEBLM2000270 | 0 | 100 | 0 | 0 | 0 | 0 | 0 |
| PEBLM2000308 | 0 | 14.901 | 0 | 3.528 | 0 | 0 | 0 |
| PEBLM2000321 | 0 | 100 | 0 | 0 | 0 | 0 | 0 |
| PEBLM2000326 | 0 | 40.802 | 0 | 0 | 0 | 0 | 0 |
| PEBLM2000333 | 0 | 100 | 0 | 0 | 0 | 0 | 0 |
| PEBLM2000338 | 0 | 71.537 | 0 | 0 | 0 | 0 | 0 |
| PEBLM2000395 | 0 | 27.366 | 6.131 | 0 | 3.228 | 0 | 0 |
| PEBLM2000479 | 0 | 34.988 | 0 | 0 | 0 | 0 | 0 |
| PEBLM2000502 | 0 | 100 | 0 | 0 | 0 | 0 | 0 |
| PEBLM2001072 | 0 | 13.631 | 0 | 0 | 6.431 | 0 | 0 |
| PEBLM2001312 | 0 | 100 | 0 | 0 | 0 | 0 | 0 |
| PEBLM2001465 | 0 | 100 | 0 | 0 | 0 | 0 | 0 |
| PEBLM2001488 | 0 | 100 | 0 | 0 | 0 | 0 | 0 |
| PEBLM2001803 | 0 | 77.704 | 0 | 0 | 0 | 0 | 0 |
| PEBLM2002432 | 0 | 100 | 0 | 0 | 0 | 0 | 0 |
| PEBLM2002455 | 0 | 66.882 | 0 | 0 | 0 | 0 | 0 |
| PEBLM2002594 | 0 | 100 | 0 | 0 | 0 | 0 | 0 |
| PEBLM2002749 | 0 | 51.656 | 0 | 0 | 0 | 0 | 0 |
| PEBLM2002887 | 0 | 100 | 0 | 0 | 0 | 0 | 0 |
| PEBLM2003426 | 0 | 100 | 0 | 0 | 0 | 0 | 0 |
| PEBLM2003765 | 0 | 100 | 0 | 0 | 0 | 0 | 0 |
| PEBLM2003914 | 0 | 15.971 | 0 | 0 | 0 | 0 | 0 |
| PEBLM2003935 | 0 | 100 | 0 | 0 | 0 | 0 | 0 |
| PEBLM2004015 | 0 | 100 | 0 | 0 | 0 | 0 | 0 |
| PEBLM2004216 | 0 | 100 | 0 | 0 | 0 | 0 | 0 |
| PEBLM2004290 | 0 | 100 | 0 | 0 | 0 | 0 | 0 |
| PEBLM2004452 | 0 | 10.145 | 0 | 4.804 | 0 | 0 | 0 |
| PEBLM2004497 | 0 | 100 | 0 | 0 | 0 | 0 | 0 |
| PEBLM2004666 | 0 | 27.928 | 0 | 6.612 | 0 | 0 | 0 |
| PEBLM2004733 | 0 | 71.830 | 0 | 0 | 0 | 0 | 0 |
| PEBLM2005282 | 0 | 88.180 | 0 | 0 | 0 | 0 | 0 |
| PEBLM2005615 | 0 | 100 | 0 | 0 | 0 | 0 | 0 |
| PEBLM2005632 | 0 | 100 | 0 | 0 | 0 | 0 | 0 |
| PEBLM2005697 | 0 | 100 | 0 | 0 | 0 | 0 | 0 |
| PEBLM2006031 | 0 | 100 | 0 | 0 | 0 | 0 | 0 |
| PEBLM2006036 | 0 | 90.014 | 0 | 0 | 0 | 0 | 0 |
| PEBLM2006049 | 0 | 100 | 0 | 0 | 0 | 0 | 0 |
| PEBLM2006113 | 0 | 100 | 0 | 0 | 0 | 0 | 0 |
| PEBLM2006135 | 0 | 100 | 0 | 0 | 0 | 0 | 0 |
| PEBLM2006215 | 0 | 100 | 0 | 0 | 0 | 0 | 0 |
| PEBLM2006283 | 0 | 88.049 | 0 | 0 | 0 | 0 | 0 |
| PEBLM2006298 | 0 | 100 | 0 | 0 | 0 | 0 | 0 |
| PEBLM2006366 | 0 | 20.804 | 0 | 0 | 0 | 0 | 0 |
| PEBLM2006709 | 0 | 41.819 | 0 | 0 | 0 | 0 | 0 |
| PEBLM2006912 | 0 | 100 | 0 | 0 | 0 | 0 | 0 |
| PEBLM2007112 | 0 | 100 | 0 | 0 | 0 | 0 | 0 |
| PEBLM2007140 | 0 | 100 | 0 | 0 | 0 | 0 | 0 |
| PEBLM2007188 | 0 | 65.617 | 0 | 0 | 0 | 0 | 0 |
| PEBLM2007296 | 0 | 25.998 | 0 | 0 | 0 | 0 | 0 |
| PEBLM2007437 | 0 | 44.815 | 0 | 0 | 0 | 33.642 | 0 |
| PEBLM2007447 | 0 | 90.014 | 0 | 0 | 0 | 0 | 0 |
| PEBLM2007598 | 0 | 100 | 0 | 0 | 0 | 0 | 0 |
| PEBLM2007774 | 0 | 4.528 | 0 | 1.072 | 1.068 | 0 | 0 |
| PEBLM2007832 | 0 | 100 | 0 | 0 | 0 | 0 | 0 |
| PEBLM2007834 | 0 | 100 | 0 | 0 | 0 | 0 | 0 |
| PEBLM2008106 | 0 | 80.142 | 0 | 0 | 0 | 0 | 0 |
| PEBLM2008576 | 0 | 100 | 0 | 0 | 0 | 0 | 0 |
| PEBLM2008605 | 0 | 100 | 0 | 0 | 0 | 0 | 0 |
| PEBLM2008826 | 0 | 69.712 | 0 | 0 | 0 | 0 | 0 |
| PEBLM2008861 | 0 | 18.093 | 0 | 0 | 0 | 0 | 0 |
| PERIC1000025 | 0 | 0 | 100 | 0 | 0 | 0 | 0 |
| PERIC1000147 | 0 | 0 | 100 | 0 | 0 | 0 | 0 |
| PERIC2000180 | 0 | 0 | 100 | 0 | 0 | 0 | 0 |
| PERIC2000214 | 0 | 0 | 100 | 0 | 0 | 0 | 0 |
| PERIC2000386 | 0 | 0 | 100 | 0 | 0 | 0 | 0 |
| PERIC2000387 | 0 | 0 | 100 | 0 | 0 | 0 | 0 |
| PERIC2000422 | 0 | 0 | 100 | 0 | 0 | 0 | 0 |
| PERIC2000473 | 0 | 0 | 42.285 | 0 | 0 | 0 | 0 |
| PERIC2000478 | 0 | 0 | 73.334 | 0 | 0 | 0 | 0 |
| PERIC2000889 | 0 | 0 | 59.923 | 0 | 0 | 0 | 0 |
| PERIC2000914 | 0 | 0 | 100 | 0 | 0 | 0 | 0 |
| PERIC2001227 | 0 | 0 | 100 | 0 | 0 | 0 | 0 |
| PERIC2001228 | 0 | 0 | 100 | 0 | 0 | 0 | 0 |
| PERIC2001379 | 0 | 0 | 100 | 0 | 0 | 0 | 0 |
| PERIC2001504 | 0 | 0 | 100 | 0 | 0 | 0 | 0 |
| PERIC2001703 | 0 | 0 | 100 | 0 | 0 | 0 | 0 |
| PERIC2001764 | 0 | 0 | 100 | 0 | 0 | 0 | 0 |
| PERIC2002243 | 0 | 0 | 100 | 0 | 0 | 0 | 0 |
| PERIC2002272 | 0 | 0 | 100 | 0 | 0 | 0 | 0 |
| PERIC2002452 | 0 | 0 | 65.624 | 0 | 0 | 0 | 0 |
| PERIC2002766 | 0 | 0 | 84.429 | 0 | 0 | 0 | 0 |
| PERIC2003001 | 0 | 0 | 100 | 0 | 0 | 0 | 0 |
| PERIC2003090 | 0 | 0 | 100 | 0 | 0 | 0 | 0 |
| PERIC2003287 | 0 | 0 | 100 | 0 | 0 | 0 | 0 |
| PERIC2003349 | 0 | 0 | 100 | 0 | 0 | 0 | 0 |
| PERIC2003452 | 0 | 0 | 100 | 0 | 0 | 0 | 0 |
| PERIC2003699 | 0 | 0 | 79.101 | 20.899 | 0 | 0 | 0 |
| PERIC2003720 | 0 | 0 | 86.988 | 0 | 0 | 0 | 0 |
| PERIC2003794 | 0 | 0 | 91.154 | 0 | 0 | 0 | 0 |
| PERIC2003834 | 0 | 0 | 100 | 0 | 0 | 0 | 0 |
| PERIC2003919 | 0 | 0 | 100 | 0 | 0 | 0 | 0 |
| PERIC2003957 | 0 | 0 | 100 | 0 | 0 | 0 | 0 |
| PERIC2004028 | 0 | 0 | 100 | 0 | 0 | 0 | 0 |
| PERIC2004131 | 0 | 0 | 100 | 0 | 0 | 0 | 0 |
| PERIC2004259 | 0 | 0 | 100 | 0 | 0 | 0 | 0 |
| PERIC2004379 | 0 | 0 | 100 | 0 | 0 | 0 | 0 |
| PERIC2004429 | 0 | 0 | 100 | 0 | 0 | 0 | 0 |
| PERIC2004510 | 0 | 0 | 100 | 0 | 0 | 0 | 0 |
| PERIC2004613 | 0 | 0 | 100 | 0 | 0 | 0 | 0 |
| PERIC2004909 | 0 | 0 | 100 | 0 | 0 | 0 | 0 |
| PERIC2005111 | 0 | 0 | 100 | 0 | 0 | 0 | 0 |
| PERIC2005117 | 0 | 0 | 14.470 | 3.823 | 0 | 0 | 0 |
| PERIC2005305 | 0 | 0 | 100 | 0 | 0 | 0 | 0 |
| PERIC2005347 | 0 | 0 | 100 | 0 | 0 | 0 | 0 |
| PERIC2005370 | 0 | 0 | 100 | 0 | 0 | 0 | 0 |
| PERIC2005936 | 0 | 0 | 100 | 0 | 0 | 0 | 0 |
| PERIC2006035 | 0 | 0 | 100 | 0 | 0 | 0 | 0 |
| PERIC2006121 | 0 | 0 | 100 | 0 | 0 | 0 | 0 |
| PERIC2006443 | 0 | 0 | 32.715 | 0 | 0 | 27.404 | 0 |
| PERIC2006945 | 0 | 0 | 100 | 0 | 0 | 0 | 0 |
| PERIC2006960 | 0 | 0 | 100 | 0 | 0 | 0 | 0 |
| PERIC2007068 | 0 | 0 | 10.394 | 0 | 0 | 0 | 0 |
| PERIC2007439 | 0 | 0 | 100 | 0 | 0 | 0 | 0 |
| PERIC2007914 | 0 | 0 | 100 | 0 | 0 | 0 | 0 |
| PERIC2008008 | 0 | 0 | 100 | 0 | 0 | 0 | 0 |
| PERIC2008385 | 0 | 0 | 100 | 0 | 0 | 0 | 0 |
| PERIC2009001 | 0 | 0 | 100 | 0 | 0 | 0 | 0 |
| PERIC2009086 | 0 | 0 | 100 | 0 | 0 | 0 | 0 |
| PERIC2009329 | 0 | 0 | 100 | 0 | 0 | 0 | 0 |
| PERIC2009566 | 0 | 0 | 100 | 0 | 0 | 0 | 0 |
| PLACE5000001 | 0 | 0 | 0 | 100 | 0 | 0 | 0 |
| PLACE5000013 | 0 | 0 | 0 | 2.825 | 0 | 0 | 0 |
| PLACE5000037 | 0 | 0 | 0 | 100 | 0 | 0 | 0 |
| PLACE5000058 | 0 | 0 | 0 | 61.610 | 0 | 0 | 0 |
| PLACE5000066 | 0 | 0 | 0 | 16.284 | 0 | 0 | 0 |
| PLACE5000067 | 0 | 0 | 0 | 73.137 | 0 | 0 | 0 |
| PLACE5000068 | 0 | 0 | 0 | 11.176 | 0 | 0 | 0 |
| PLACE5000070 | 0 | 0 | 0 | 100 | 0 | 0 | 0 |
| PLACE5000080 | 0 | 0 | 0 | 21.952 | 0 | 0 | 0 |
| PLACE5000105 | 0 | 0 | 0 | 100 | 0 | 0 | 0 |
| PLACE5000113 | 0 | 0 | 0 | 12.248 | 0 | 0 | 0 |
| PLACE5000115 | 0 | 0 | 0 | 100 | 0 | 0 | 0 |
| PLACE5000116 | 0 | 0 | 0 | 100 | 0 | 0 | 0 |
| PLACE5000129 | 0 | 0 | 0 | 100 | 0 | 0 | 0 |
| PLACE5000139 | 0 | 0 | 79.101 | 20.899 | 0 | 0 | 0 |
| PLACE5000153 | 0 | 0 | 0 | 100 | 0 | 0 | 0 |
| PLACE5000159 | 0 | 0 | 0 | 4.045 | 0 | 0 | 0 |
| PLACE5000170 | 0 | 0 | 0 | 30.938 | 0 | 0 | 0 |
| PLACE5000171 | 0 | 0 | 0 | 100 | 0 | 0 | 0 |
| PLACE5000172 | 0 | 0 | 0 | 27.759 | 0 | 0 | 0 |
| PLACE5000178 | 0 | 0 | 0 | 64.025 | 0 | 0 | 0 |
| PLACE5000245 | 0 | 0 | 0 | 100 | 0 | 0 | 0 |
| PLACE5000260 | 0 | 0 | 0 | 100 | 0 | 0 | 0 |
| PLACE5000282 | 0 | 0 | 0 | 21.952 | 0 | 0 | 0 |
| PLACE5000297 | 0 | 0 | 0 | 63.562 | 0 | 0 | 0 |
| PLACE5000372 | 0 | 0 | 0 | 59.874 | 0 | 0 | 0 |
| PLACE5000492 | 0 | 0 | 0 | 43.685 | 0 | 0 | 0 |
| PLACE5000522 | 0 | 0 | 0 | 100 | 0 | 0 | 0 |
| PLACE5000527 | 0 | 0 | 0 | 100 | 0 | 0 | 0 |
| PLACE6000012 | 0 | 0 | 0 | 100 | 0 | 0 | 0 |
| PLACE6000044 | 0 | 0 | 0 | 100 | 0 | 0 | 0 |
| PLACE6000055 | 0 | 0 | 0 | 100 | 0 | 0 | 0 |
| PLACE6000070 | 0 | 0 | 0 | 100 | 0 | 0 | 0 |
| PLACE6000137 | 0 | 0 | 0 | 100 | 0 | 0 | 0 |
| PLACE6000145 | 0 | 0 | 0 | 1.844 | 0 | 0 | 0 |
| PLACE6000191 | 0 | 0 | 0 | 100 | 0 | 0 | 0 |
| PLACE6000205 | 0 | 0 | 0 | 100 | 0 | 0 | 0 |
| PLACE6000231 | 0 | 0 | 0 | 100 | 0 | 0 | 0 |
| PLACE6000263 | 0 | 0 | 10.241 | 2.706 | 0 | 0 | 0 |
| PLACE6000296 | 0 | 0 | 0 | 100 | 0 | 0 | 0 |
| PLACE6000314 | 0 | 0 | 0 | 27.144 | 0 | 0 | 0 |
| PLACE6000325 | 0 | 0 | 0 | 14.417 | 0 | 0 | 0 |
| PLACE6000348 | 0 | 0 | 0 | 50.674 | 0 | 0 | 0 |
| PLACE6000371 | 0 | 0 | 0 | 50.569 | 0 | 0 | 0 |
| PLACE6000379 | 0 | 3.983 | 0 | 1.257 | 0 | 2.990 | 0 |
| PLACE6000414 | 0 | 0 | 0 | 2.280 | 4.544 | 0 | 0 |
| PLACE6000424 | 0 | 0 | 0 | 100 | 0 | 0 | 0 |
| PLACE6000426 | 0 | 0 | 0 | 100 | 0 | 0 | 0 |
| PLACE6000429 | 0 | 0 | 0 | 100 | 0 | 0 | 0 |
| PLACE6000450 | 0 | 0 | 0 | 100 | 0 | 0 | 0 |
| PLACE6000463 | 0 | 0 | 0 | 13.886 | 0 | 0 | 0 |
| PLACE6000523 | 0 | 0 | 0 | 100 | 0 | 0 | 0 |
| PLACE6000550 | 0 | 0 | 0 | 100 | 0 | 0 | 0 |
| PLACE6000552 | 0 | 0 | 0 | 100 | 0 | 0 | 0 |
| PLACE6000555 | 0 | 0 | 0 | 100 | 0 | 0 | 0 |
| PLACE6000619 | 0 | 0 | 0 | 63.850 | 0 | 0 | 0 |
| PLACE6000630 | 0 | 0 | 0 | 6.867 | 0 | 0 | 0 |
| PLACE6000953 | 0 | 0 | 0 | 100 | 0 | 0 | 0 |
| PLACE6001064 | 0 | 4.312 | 0 | 3.063 | 0 | 0 | 0 |
| PLACE6001118 | 0 | 0 | 0 | 100 | 0 | 0 | 0 |
| PLACE6001153 | 0 | 0 | 0 | 100 | 0 | 0 | 0 |
| PLACE6001185 | 0 | 0 | 0 | 38.010 | 0 | 0 | 0 |
| PLACE6001262 | 0 | 0 | 0 | 15.551 | 0 | 0 | 0 |
| PLACE6001281 | 0 | 0 | 0 | 6.127 | 0 | 19.425 | 0 |
| PLACE6001294 | 0 | 0 | 0 | 100 | 0 | 0 | 0 |
| PLACE6001443 | 0 | 0 | 0 | 10.487 | 0 | 0 | 0 |
| PLACE6001536 | 0 | 0 | 0 | 100 | 0 | 0 | 0 |
| PLACE6001557 | 0 | 0 | 0 | 100 | 0 | 0 | 0 |
| PLACE6001712 | 0 | 0 | 0 | 100 | 0 | 0 | 0 |
| PLACE6001740 | 0 | 0 | 0 | 100 | 0 | 0 | 0 |
| PLACE6001823 | 0 | 0 | 0 | 50.708 | 0 | 0 | 0 |
| PLACE6001886 | 0 | 0 | 0 | 30.140 | 0 | 0 | 0 |
| PLACE6001923 | 0 | 0 | 0 | 11.186 | 0 | 0 | 0 |
| PLACE6001925 | 0 | 0 | 0 | 100 | 0 | 0 | 0 |
| PLACE6001933 | 0 | 0 | 0 | 100 | 0 | 0 | 0 |
| PLACE6002011 | 0 | 0 | 0 | 50.569 | 0 | 0 | 0 |
| PLACE6002016 | 0 | 0 | 0 | 100 | 0 | 0 | 0 |
| PLACE6002056 | 0 | 0 | 0 | 50.569 | 0 | 0 | 0 |
| PLACE6002084 | 0 | 2.776 | 2.488 | 0.657 | 0 | 4.168 | 0 |
| PLACE6002102 | 0 | 0 | 0 | 7.541 | 0 | 0 | 0 |
| PLACE6002157 | 0 | 0 | 0 | 100 | 0 | 0 | 0 |
| PLACE6002312 | 0 | 0 | 0 | 100 | 0 | 0 | 0 |
| PLACE6002323 | 0 | 0 | 0 | 100 | 0 | 0 | 0 |
| PLACE6002345 | 0 | 0 | 0 | 100 | 0 | 0 | 0 |
| PLACE6002419 | 0 | 0 | 0 | 18.553 | 0 | 0 | 0 |
| PLACE6002462 | 0 | 0 | 0 | 100 | 0 | 0 | 0 |
| PLACE6002647 | 0 | 0 | 0 | 100 | 0 | 0 | 0 |
| PLACE6002668 | 0 | 0 | 0 | 9.604 | 0 | 0 | 0 |
| PLACE6002692 | 0 | 0 | 0 | 63.850 | 0 | 0 | 0 |
| PLACE6002699 | 0 | 0 | 0 | 100 | 0 | 0 | 0 |
| PLACE6002710 | 0 | 0 | 0 | 100 | 0 | 0 | 0 |
| PLACE6002871 | 0 | 0 | 0 | 100 | 0 | 0 | 0 |
| PLACE6002949 | 0 | 0 | 0 | 100 | 0 | 0 | 0 |
| PLACE6002960 | 0 | 0 | 7.476 | 25.678 | 0 | 12.525 | 0 |
| PLACE6003004 | 0 | 0 | 0 | 61.610 | 0 | 0 | 0 |
| PLACE6003038 | 0 | 27.127 | 0 | 6.423 | 12.799 | 0 | 0 |
| PLACE6003077 | 0 | 0 | 0 | 45.210 | 0 | 0 | 0 |
| PLACE6003094 | 0 | 0 | 0 | 100 | 0 | 0 | 0 |
| PLACE6003109 | 0 | 0 | 0 | 100 | 0 | 0 | 0 |
| PLACE6003204 | 0 | 0 | 0 | 78.067 | 0 | 0 | 0 |
| PLACE6003218 | 0 | 0 | 0 | 51.794 | 0 | 0 | 0 |
| PLACE6003372 | 0 | 0 | 0 | 100 | 0 | 0 | 0 |
| PLACE6003383 | 0 | 0 | 0 | 2.124 | 12.698 | 0 | 0 |
| PLACE6003393 | 0 | 0 | 0 | 13.514 | 0 | 0 | 0 |
| PLACE6003399 | 0 | 0 | 0 | 100 | 0 | 0 | 0 |
| PLACE6003539 | 0 | 0 | 0 | 100 | 0 | 0 | 0 |
| PLACE6003621 | 0 | 0 | 0 | 71.489 | 0 | 0 | 0 |
| PLACE6003705 | 0 | 0 | 0 | 100 | 0 | 0 | 0 |
| PLACE6003740 | 0 | 0 | 0 | 100 | 0 | 0 | 0 |
| PLACE6003745 | 0 | 0 | 0 | 28.055 | 0 | 29.650 | 0 |
| PLACE6003771 | 0 | 0 | 0 | 100 | 0 | 0 | 0 |
| PLACE6003796 | 0 | 0 | 0 | 100 | 0 | 0 | 0 |
| PLACE6003839 | 0 | 0 | 0 | 100 | 0 | 0 | 0 |
| PLACE6003850 | 0 | 0 | 0 | 19.965 | 0 | 0 | 0 |
| PLACE6004005 | 0 | 0 | 0 | 1.757 | 0 | 0 | 0 |
| PLACE6004101 | 0 | 0 | 0 | 7.929 | 0 | 0 | 0 |
| PLACE6004219 | 0 | 0 | 0 | 100 | 0 | 0 | 0 |
| PLACE6004312 | 0 | 0 | 0 | 2.147 | 2.139 | 0 | 0 |
| PLACE6004336 | 0 | 0 | 0 | 100 | 0 | 0 | 0 |
| PLACE6004368 | 0 | 0 | 0 | 100 | 0 | 0 | 0 |
| PLACE6004380 | 0 | 0 | 0 | 100 | 0 | 0 | 0 |
| PLACE6004396 | 0 | 0 | 0 | 32.670 | 0 | 0 | 0 |
| PLACE6004397 | 0 | 13.816 | 0 | 3.271 | 0 | 10.371 | 0 |
| PLACE6004454 | 0 | 0 | 0 | 100 | 0 | 0 | 0 |
| PLACE6004464 | 0 | 0 | 0 | 67.171 | 0 | 0 | 0 |
| PLACE6004491 | 0 | 0 | 0 | 59.874 | 0 | 0 | 0 |
| PLACE6004578 | 0 | 0 | 0 | 6.618 | 0 | 0 | 0 |
| PLACE6004663 | 0 | 0 | 0 | 100 | 0 | 0 | 0 |
| PLACE6004687 | 0 | 0 | 0 | 100 | 0 | 0 | 0 |
| PLACE6004738 | 0 | 0 | 0 | 74.902 | 0 | 0 | 0 |
| PLACE6004931 | 0 | 0 | 0 | 11.653 | 0 | 0 | 0 |
| PLACE6004966 | 0 | 0 | 0 | 100 | 0 | 0 | 0 |
| PLACE6004993 | 0 | 0 | 0 | 33.936 | 0 | 0 | 0 |
| PLACE6005007 | 0 | 0 | 0 | 100 | 0 | 0 | 0 |
| PLACE6005029 | 0 | 0 | 0 | 100 | 0 | 0 | 0 |
| PLACE6005108 | 0 | 0 | 0 | 100 | 0 | 0 | 0 |
| PLACE6005231 | 0 | 0 | 0 | 19.656 | 0 | 0 | 0 |
| PLACE6005234 | 0 | 0 | 0 | 100 | 0 | 0 | 0 |
| PLACE6005283 | 0 | 0 | 0 | 100 | 0 | 0 | 0 |
| PLACE6005294 | 0 | 0 | 0 | 21.429 | 0 | 0 | 0 |
| PLACE6005328 | 0 | 0 | 0 | 100 | 0 | 0 | 0 |
| PLACE6005351 | 0 | 0 | 0 | 100 | 0 | 0 | 0 |
| PLACE6005423 | 0 | 0 | 0 | 100 | 0 | 0 | 0 |
| PLACE6005482 | 0 | 0 | 0 | 9.412 | 0 | 0 | 0 |
| PLACE6005487 | 0 | 0 | 0 | 6.658 | 0 | 0 | 0 |
| PLACE6005535 | 0 | 0 | 0 | 100 | 0 | 0 | 0 |
| PLACE6005546 | 0 | 0 | 0 | 100 | 0 | 0 | 0 |
| PLACE6005559 | 0 | 0 | 0 | 100 | 0 | 0 | 0 |
| PLACE6005579 | 0 | 0 | 0 | 100 | 0 | 0 | 0 |
| PLACE6005691 | 0 | 0 | 0 | 63.850 | 0 | 0 | 0 |
| PLACE6005786 | 0 | 0 | 0 | 100 | 0 | 0 | 0 |
| PLACE6006042 | 0 | 0 | 0 | 9.688 | 0 | 0 | 0 |
| PLACE6006077 | 0 | 0 | 0 | 100 | 0 | 0 | 0 |
| PLACE6006137 | 0 | 0 | 0 | 6.079 | 0 | 0 | 0 |
| PLACE6006158 | 0 | 0 | 0 | 100 | 0 | 0 | 0 |
| PLACE6006186 | 0 | 0 | 0 | 12.872 | 0 | 0 | 0 |
| PLACE6006217 | 0 | 0 | 0 | 100 | 0 | 0 | 0 |
| PLACE6006222 | 0 | 0 | 0 | 14.870 | 0 | 0 | 0 |
| PLACE6006266 | 0 | 0 | 0 | 100 | 0 | 0 | 0 |
| PLACE6006287 | 0 | 0 | 0 | 100 | 0 | 0 | 0 |
| PLACE6006394 | 0 | 0 | 0 | 100 | 0 | 0 | 0 |
| PLACE6006418 | 0 | 0 | 0 | 9.868 | 0 | 0 | 0 |
| PLACE6006474 | 0 | 0 | 0 | 100 | 0 | 0 | 0 |
| PLACE6006549 | 0 | 0 | 0 | 2.842 | 5.664 | 0 | 0 |
| PLACE6006566 | 0 | 0 | 0 | 100 | 0 | 0 | 0 |
| PLACE6006697 | 0 | 0 | 0 | 100 | 0 | 0 | 0 |
| PLACE6006822 | 0 | 0 | 0 | 100 | 0 | 0 | 0 |
| PLACE6006871 | 0 | 0 | 0 | 100 | 0 | 0 | 0 |
| PLACE6006905 | 0 | 0 | 0 | 100 | 0 | 0 | 0 |
| PLACE6006988 | 0 | 0 | 0 | 58.892 | 0 | 0 | 0 |
| PLACE6007050 | 0 | 0 | 0 | 100 | 0 | 0 | 0 |
| PLACE6007180 | 0 | 0 | 0 | 19.073 | 0 | 0 | 0 |
| PLACE6007239 | 0 | 0 | 0 | 95.382 | 0 | 0 | 0 |
| PLACE6007242 | 0 | 0 | 0 | 100 | 0 | 0 | 0 |
| PLACE6007309 | 0 | 0 | 0 | 32.348 | 0 | 0 | 0 |
| PLACE6007373 | 0 | 0 | 0 | 5.093 | 0 | 0 | 0 |
| PLACE6007380 | 0 | 0 | 0 | 100 | 0 | 0 | 0 |
| PLACE6007433 | 0 | 0 | 0 | 100 | 0 | 0 | 0 |
| PLACE6007436 | 0 | 0 | 0 | 19.181 | 0 | 0 | 0 |
| PLACE6007482 | 0 | 0 | 0 | 100 | 0 | 0 | 0 |
| PLACE6007687 | 0 | 0 | 0 | 100 | 0 | 0 | 0 |
| PLACE6007787 | 0 | 0 | 0 | 100 | 0 | 0 | 0 |
| PLACE6007798 | 0 | 0 | 0 | 100 | 0 | 0 | 0 |
| PLACE6007807 | 0 | 0 | 0 | 100 | 0 | 0 | 0 |
| PLACE6007925 | 0 | 0 | 0 | 18.034 | 0 | 0 | 0 |
| PLACE6008036 | 0 | 0 | 0 | 100 | 0 | 0 | 0 |
| PLACE6008094 | 0 | 0 | 0 | 100 | 0 | 0 | 0 |
| PLACE6008126 | 0 | 0 | 0 | 100 | 0 | 0 | 0 |
| PLACE6008285 | 0 | 0 | 0 | 100 | 0 | 0 | 0 |
| PLACE6008444 | 0 | 0 | 0 | 63.850 | 0 | 0 | 0 |
| PLACE6008453 | 0 | 0 | 0 | 100 | 0 | 0 | 0 |
| PLACE6008640 | 0 | 0 | 0 | 100 | 0 | 0 | 0 |
| PLACE6008750 | 0 | 0 | 0 | 100 | 0 | 0 | 0 |
| PLACE6008768 | 0 | 0 | 0 | 100 | 0 | 0 | 0 |
| PLACE6008775 | 0 | 0 | 0 | 6.560 | 0 | 0 | 0 |
| PLACE6008793 | 0 | 0 | 0 | 2.822 | 0 | 0 | 0 |
| PLACE6008824 | 0 | 0 | 0 | 59.874 | 0 | 0 | 0 |
| PLACE6008989 | 0 | 0 | 0 | 100 | 0 | 0 | 0 |
| PLACE6009006 | 0 | 0 | 0 | 100 | 0 | 0 | 0 |
| PLACE6009008 | 0 | 0 | 0 | 33.415 | 66.585 | 0 | 0 |
| PLACE6009228 | 0 | 0 | 0 | 50.569 | 0 | 0 | 0 |
| PLACE6009237 | 0 | 0 | 0 | 7.653 | 0 | 0 | 0 |
| PLACE6009338 | 0 | 0 | 0 | 44.716 | 0 | 0 | 0 |
| PLACE6009419 | 0 | 0 | 0 | 100 | 0 | 0 | 0 |
| PLACE6009430 | 0 | 0 | 0 | 100 | 0 | 0 | 0 |
| PLACE6009482 | 0 | 0 | 0 | 100 | 0 | 0 | 0 |
| PLACE6009524 | 0 | 0 | 0 | 100 | 0 | 0 | 0 |
| PLACE6009527 | 0 | 0 | 0 | 100 | 0 | 0 | 0 |
| PLACE6009560 | 0 | 0 | 0 | 19.232 | 0 | 0 | 0 |
| PLACE6009590 | 0 | 0 | 0 | 100 | 0 | 0 | 0 |
| PLACE6009821 | 0 | 0 | 0 | 27.404 | 0 | 0 | 0 |
| PLACE6009835 | 0 | 0 | 0 | 100 | 0 | 0 | 0 |
| PLACE6009853 | 0 | 0 | 0 | 100 | 0 | 0 | 0 |
| PLACE6010077 | 0 | 0 | 0 | 4.648 | 0 | 0 | 0 |
| PLACE6010463 | 0 | 0 | 0 | 100 | 0 | 0 | 0 |
| PLACE6010484 | 0 | 11.618 | 0 | 2.751 | 5.482 | 0 | 0 |
| PLACE6010485 | 0 | 0 | 0 | 100 | 0 | 0 | 0 |
| PLACE6010568 | 0 | 0 | 0 | 100 | 0 | 0 | 0 |
| PLACE6010701 | 0 | 0 | 0 | 33.415 | 66.585 | 0 | 0 |
| PLACE6010704 | 0 | 0 | 0 | 64.025 | 0 | 0 | 0 |
| PLACE6010732 | 0 | 0 | 0 | 43.685 | 0 | 0 | 0 |
| PLACE6010765 | 0 | 0 | 79.101 | 20.899 | 0 | 0 | 0 |
| PLACE6010848 | 0 | 0 | 0 | 14.563 | 0 | 0 | 0 |
| PLACE6010925 | 0 | 0 | 0 | 100 | 0 | 0 | 0 |
| PLACE6010936 | 0 | 0 | 0 | 87.601 | 0 | 0 | 0 |
| PLACE6010991 | 0 | 0 | 0 | 100 | 0 | 0 | 0 |
| PLACE6011041 | 0 | 0 | 0 | 29.052 | 0 | 0 | 0 |
| PLACE6011057 | 0 | 0 | 0 | 8.022 | 0 | 0 | 0 |
| PLACE6011102 | 0 | 0 | 0 | 100 | 0 | 0 | 0 |
| PLACE6011156 | 0 | 0 | 0 | 23.174 | 0 | 0 | 0 |
| PLACE6011211 | 0 | 0 | 0 | 100 | 0 | 0 | 0 |
| PLACE6011260 | 0 | 0 | 0 | 100 | 0 | 0 | 0 |
| PLACE6011334 | 0 | 0 | 0 | 15.995 | 0 | 0 | 0 |
| PLACE6011627 | 0 | 0 | 0 | 58.892 | 0 | 0 | 0 |
| PLACE6011774 | 0 | 0 | 0 | 100 | 0 | 0 | 0 |
| PLACE6011881 | 0 | 0 | 0 | 100 | 0 | 0 | 0 |
| PLACE6011907 | 0 | 0 | 0 | 100 | 0 | 0 | 0 |
| PLACE6011970 | 0 | 0 | 0 | 46.156 | 0 | 0 | 0 |
| PLACE6011975 | 0 | 0 | 0 | 100 | 0 | 0 | 0 |
| PLACE6012028 | 0 | 0 | 0 | 11.172 | 0 | 0 | 0 |
| PLACE6012108 | 0 | 0 | 0 | 100 | 0 | 0 | 0 |
| PLACE6012168 | 0 | 0 | 0 | 100 | 0 | 0 | 0 |
| PLACE6012297 | 0 | 0 | 0 | 100 | 0 | 0 | 0 |
| PLACE6012308 | 0 | 0 | 0 | 100 | 0 | 0 | 0 |
| PLACE6012574 | 0 | 0 | 0 | 12.733 | 0 | 0 | 0 |
| PLACE6012908 | 0 | 0 | 0 | 100 | 0 | 0 | 0 |
| PLACE6012936 | 0 | 0 | 0 | 68.095 | 0 | 0 | 0 |
| PLACE6012942 | 0 | 0 | 0 | 59.874 | 0 | 0 | 0 |
| PLACE6013222 | 0 | 0 | 0 | 17.538 | 0 | 0 | 0 |
| PLACE6013232 | 0 | 0 | 0 | 20.563 | 0 | 0 | 0 |
| PLACE6013288 | 0 | 0 | 0 | 100 | 0 | 0 | 0 |
| PLACE6013386 | 0 | 0 | 0 | 100 | 0 | 0 | 0 |
| PLACE6013400 | 0 | 0 | 0 | 100 | 0 | 0 | 0 |
| PLACE6013650 | 0 | 0 | 0 | 100 | 0 | 0 | 0 |
| PLACE6013672 | 0 | 0 | 0 | 49.147 | 0 | 0 | 0 |
| PLACE6013883 | 0 | 0 | 0 | 61.610 | 0 | 0 | 0 |
| PLACE6013884 | 0 | 0 | 0 | 100 | 0 | 0 | 0 |
| PLACE6013885 | 0 | 0 | 0 | 100 | 0 | 0 | 0 |
| PLACE6014043 | 0 | 0 | 0 | 100 | 0 | 0 | 0 |
| PLACE6014064 | 0 | 0 | 0 | 5.447 | 0 | 0 | 66.593 |
| PLACE6014516 | 0 | 0 | 0 | 100 | 0 | 0 | 0 |
| PLACE6014882 | 0 | 0 | 0 | 21.139 | 0 | 0 | 0 |
| PLACE6015051 | 0 | 0 | 0 | 8.204 | 0 | 0 | 0 |
| PLACE6015211 | 0 | 0 | 0 | 100 | 0 | 0 | 0 |
| PLACE6015322 | 0 | 0 | 0 | 50.569 | 0 | 0 | 0 |
| PLACE6015445 | 0 | 0 | 0 | 19.777 | 0 | 0 | 0 |
| PLACE6015460 | 0 | 0 | 0 | 100 | 0 | 0 | 0 |
| PLACE6015513 | 0 | 0 | 0 | 45.210 | 0 | 0 | 0 |
| PLACE6015591 | 0 | 0 | 0 | 100 | 0 | 0 | 0 |
| PLACE6015729 | 0 | 0 | 0 | 63.850 | 0 | 0 | 0 |
| PLACE6015731 | 0 | 0 | 0 | 27.317 | 0 | 0 | 0 |
| PLACE6015939 | 0 | 0 | 0 | 100 | 0 | 0 | 0 |
| PLACE6016030 | 0 | 0 | 0 | 100 | 0 | 0 | 0 |
| PLACE6016093 | 0 | 0 | 0 | 100 | 0 | 0 | 0 |
| PLACE6016105 | 0 | 0 | 0 | 100 | 0 | 0 | 0 |
| PLACE6016160 | 0 | 0 | 0 | 100 | 0 | 0 | 0 |
| PLACE6016210 | 0 | 0 | 0 | 22.537 | 0 | 0 | 0 |
| PLACE6016254 | 0 | 0 | 0 | 32.516 | 0 | 0 | 0 |
| PLACE6016383 | 0 | 0 | 0 | 11.505 | 0 | 0 | 0 |
| PLACE6016942 | 0 | 0 | 0 | 6.962 | 0 | 0 | 0 |
| PLACE6016984 | 0 | 0 | 0 | 65.750 | 0 | 0 | 0 |
| PLACE6017002 | 0 | 0 | 0 | 100 | 0 | 0 | 0 |
| PLACE6017431 | 0 | 0 | 0 | 59.874 | 0 | 0 | 0 |
| PLACE6017564 | 0 | 0 | 0 | 100 | 0 | 0 | 0 |
| PLACE6017578 | 0 | 0 | 0 | 36.001 | 0 | 0 | 0 |
| PLACE6017626 | 0 | 0 | 0 | 50.519 | 0 | 0 | 0 |
| PLACE6017701 | 0 | 0 | 0 | 43.685 | 0 | 0 | 0 |
| PLACE6017714 | 0 | 0 | 0 | 100 | 0 | 0 | 0 |
| PLACE6017788 | 0 | 0 | 0 | 73.137 | 0 | 0 | 0 |
| PLACE6017791 | 0 | 0 | 0 | 19.547 | 0 | 0 | 0 |
| PLACE6018031 | 0 | 0 | 0 | 11.966 | 0 | 0 | 0 |
| PLACE6018107 | 0 | 0 | 0 | 100 | 0 | 0 | 0 |
| PLACE6018187 | 0 | 0 | 0 | 100 | 0 | 0 | 0 |
| PLACE6018235 | 0 | 0 | 0 | 100 | 0 | 0 | 0 |
| PLACE6018287 | 0 | 0 | 0 | 84.484 | 0 | 0 | 0 |
| PLACE6018441 | 0 | 0 | 0 | 100 | 0 | 0 | 0 |
| PLACE6018487 | 0 | 0 | 0 | 1.188 | 0 | 0 | 0 |
| PLACE6018497 | 0 | 0 | 0 | 100 | 0 | 0 | 0 |
| PLACE6018769 | 0 | 0 | 0 | 100 | 0 | 0 | 0 |
| PLACE6018834 | 0 | 0 | 0 | 100 | 0 | 0 | 0 |
| PLACE6018843 | 0 | 0 | 0 | 100 | 0 | 0 | 0 |
| PLACE6018863 | 0 | 0 | 0 | 33.796 | 0 | 0 | 0 |
| PLACE6018938 | 0 | 0 | 0 | 54.615 | 0 | 0 | 0 |
| PLACE6019195 | 0 | 0 | 0 | 30.202 | 0 | 0 | 0 |
| PLACE6019385 | 0 | 0 | 0 | 7.219 | 0 | 0 | 0 |
| PLACE6019542 | 0 | 0 | 0 | 100 | 0 | 0 | 0 |
| PLACE6019600 | 0 | 0 | 0 | 100 | 0 | 0 | 0 |
| PLACE6019674 | 0 | 0 | 0 | 100 | 0 | 0 | 0 |
| PLACE6019676 | 0 | 0 | 0 | 100 | 0 | 0 | 0 |
| PLACE6019701 | 0 | 0 | 0 | 100 | 0 | 0 | 0 |
| PLACE6019932 | 0 | 0 | 0 | 7.719 | 0 | 0 | 0 |
| PLACE6020031 | 0 | 0 | 0 | 100 | 0 | 0 | 0 |
| PLACE6020145 | 0 | 0 | 0 | 14.936 | 0 | 0 | 0 |
| PLACE7000167 | 0 | 0 | 0 | 100 | 0 | 0 | 0 |
| PLACE7000170 | 0 | 0 | 0 | 100 | 0 | 0 | 0 |
| PLACE7000266 | 0 | 0 | 0 | 100 | 0 | 0 | 0 |
| PLACE7000333 | 0 | 0 | 0 | 100 | 0 | 0 | 0 |
| PLACE7000410 | 0 | 0 | 0 | 61.610 | 0 | 0 | 0 |
| PLACE7000502 | 0 | 0 | 0 | 100 | 0 | 0 | 0 |
| PLACE7000707 | 0 | 0 | 0 | 100 | 0 | 0 | 0 |
| PLACE7001022 | 0 | 0 | 0 | 50.083 | 0 | 0 | 0 |
| PLACE7001544 | 0 | 0 | 0 | 100 | 0 | 0 | 0 |
| PLACE7001759 | 0 | 0 | 0 | 100 | 0 | 0 | 0 |
| PLACE7001936 | 0 | 0 | 0 | 100 | 0 | 0 | 0 |
| PLACE7002303 | 0 | 0 | 0 | 100 | 0 | 0 | 0 |
| PLACE7002641 | 0 | 0 | 0 | 100 | 0 | 0 | 0 |
| PLACE7003639 | 0 | 0 | 0 | 100 | 0 | 0 | 0 |
| PLACE7003657 | 0 | 0 | 0 | 100 | 0 | 0 | 0 |
| PLACE7003684 | 0 | 0 | 0 | 100 | 0 | 0 | 0 |
| PLACE7003985 | 0 | 0 | 0 | 100 | 0 | 0 | 0 |
| PLACE7004103 | 0 | 0 | 0 | 11.508 | 0 | 0 | 0 |
| PLACE7004961 | 0 | 0 | 0 | 100 | 0 | 0 | 0 |
| PLACE7005169 | 0 | 0 | 0 | 100 | 0 | 0 | 0 |
| PLACE7005671 | 0 | 0 | 0 | 100 | 0 | 0 | 0 |
| PLACE7005840 | 0 | 0 | 0 | 68.095 | 0 | 0 | 0 |
| PLACE7006051 | 0 | 0 | 0 | 100 | 0 | 0 | 0 |
| PLACE7006090 | 0 | 0 | 0 | 100 | 0 | 0 | 0 |
| PLACE7006240 | 0 | 0 | 0 | 44.802 | 0 | 0 | 0 |
| PLACE7006268 | 0 | 0 | 0 | 33.415 | 66.585 | 0 | 0 |
| PLACE7006275 | 0 | 0 | 0 | 100 | 0 | 0 | 0 |
| PLACE7006498 | 0 | 0 | 0 | 100 | 0 | 0 | 0 |
| PLACE7006540 | 0 | 0 | 0 | 100 | 0 | 0 | 0 |
| PLACE7007379 | 0 | 0 | 0 | 100 | 0 | 0 | 0 |
| PLACE7007644 | 0 | 0 | 0 | 100 | 0 | 0 | 0 |
| PLACE7007973 | 0 | 0 | 0 | 100 | 0 | 0 | 0 |
| PLACE7008136 | 0 | 0 | 0 | 100 | 0 | 0 | 0 |
| PLACE7008261 | 0 | 0 | 0 | 50.708 | 0 | 0 | 0 |
| PLACE7008431 | 0 | 0 | 0 | 100 | 0 | 0 | 0 |
| PLACE7008623 | 0 | 0 | 0 | 100 | 0 | 0 | 0 |
| PLACE7008766 | 0 | 0 | 0 | 100 | 0 | 0 | 0 |
| PLACE7009563 | 0 | 0 | 0 | 100 | 0 | 0 | 0 |
| PROST1000033 | 0 | 0 | 0 | 0 | 39.071 | 0 | 0 |
| PROST1000039 | 0 | 0 | 0 | 0 | 100 | 0 | 0 |
| PROST1000065 | 0 | 0 | 0 | 0 | 100 | 0 | 0 |
| PROST1000083 | 0 | 0 | 0 | 0 | 100 | 0 | 0 |
| PROST1000085 | 0 | 0 | 0 | 0 | 24.534 | 0 | 0 |
| PROST1000097 | 0 | 0 | 7.922 | 2.093 | 2.085 | 0 | 0 |
| PROST1000110 | 0 | 0 | 0 | 0 | 100 | 0 | 0 |
| PROST1000120 | 0 | 24.787 | 0 | 5.869 | 11.695 | 18.607 | 0 |
| PROST1000123 | 0 | 0 | 0 | 0 | 100 | 0 | 0 |
| PROST1000136 | 0 | 0 | 0 | 0 | 100 | 0 | 0 |
| PROST1000144 | 0 | 0 | 0 | 2.790 | 5.560 | 0 | 0 |
| PROST1000145 | 0 | 0 | 0 | 0 | 100 | 0 | 0 |
| PROST1000152 | 0 | 0 | 0 | 0 | 100 | 0 | 0 |
| PROST1000167 | 0 | 0 | 0 | 0 | 100 | 0 | 0 |
| PROST1000184 | 0 | 0 | 0 | 0 | 100 | 0 | 0 |
| PROST1000199 | 0 | 0 | 0 | 1.300 | 5.182 | 0 | 0 |
| PROST1000215 | 0 | 0 | 0 | 0 | 18.139 | 28.860 | 0 |
| PROST1000217 | 0 | 0 | 0 | 0 | 100 | 0 | 0 |
| PROST1000220 | 0 | 0 | 0 | 0 | 43.340 | 0 | 0 |
| PROST1000226 | 0 | 0 | 0 | 6.368 | 12.690 | 0 | 0 |
| PROST1000246 | 0 | 0 | 0 | 0 | 38.964 | 0 | 0 |
| PROST1000262 | 0 | 0 | 0 | 0 | 100 | 0 | 0 |
| PROST1000272 | 0 | 0 | 0 | 0 | 11.190 | 0 | 0 |
| PROST1000298 | 0 | 0 | 0 | 0 | 100 | 0 | 0 |
| PROST1000322 | 0 | 0 | 0 | 0 | 42.387 | 0 | 0 |
| PROST1000334 | 0 | 0 | 0 | 0 | 100 | 0 | 0 |
| PROST1000343 | 0 | 0 | 0 | 0 | 10.074 | 0 | 0 |
| PROST1000362 | 0 | 0 | 0 | 0 | 100 | 0 | 0 |
| PROST1000377 | 0 | 0 | 0 | 0 | 100 | 0 | 0 |
| PROST1000383 | 0 | 0 | 0 | 0 | 76.179 | 0 | 0 |
| PROST1000412 | 0 | 0 | 0 | 0 | 100 | 0 | 0 |
| PROST1000419 | 0 | 0 | 0 | 0 | 100 | 0 | 0 |
| PROST1000451 | 0 | 0 | 0 | 0 | 100 | 0 | 0 |
| PROST1000480 | 0 | 0 | 0 | 0 | 67.090 | 0 | 0 |
| PROST1000485 | 0 | 0 | 0 | 0 | 100 | 0 | 0 |
| PROST1000493 | 0 | 0 | 0 | 0 | 100 | 0 | 0 |
| PROST1000512 | 0 | 0 | 0 | 0 | 53.436 | 0 | 0 |
| PROST1000526 | 0 | 0 | 0 | 0 | 8.960 | 0 | 0 |
| PROST1000527 | 0 | 0 | 0 | 0 | 100 | 0 | 0 |
| PROST1000528 | 0 | 0 | 0 | 0 | 100 | 0 | 0 |
| PROST1000536 | 0 | 0 | 0 | 10.700 | 42.645 | 0 | 0 |
| PROST1000559 | 0 | 0 | 0 | 0 | 100 | 0 | 0 |
| PROST1000564 | 0 | 0 | 0 | 0 | 4.767 | 0 | 0 |
| PROST1000575 | 0 | 0 | 0 | 0 | 100 | 0 | 0 |
| PROST2000036 | 0 | 0 | 0 | 0 | 9.969 | 0 | 0 |
| PROST2000053 | 0 | 0 | 0 | 0 | 100 | 0 | 0 |
| PROST2000067 | 0 | 0 | 0 | 0 | 100 | 0 | 0 |
| PROST2000079 | 0 | 0 | 0 | 0 | 10.615 | 0 | 0 |
| PROST2000105 | 0 | 0 | 0 | 0 | 100 | 0 | 0 |
| PROST2000138 | 0 | 0.651 | 1.168 | 3.085 | 1.537 | 0 | 0 |
| PROST2000143 | 0 | 0 | 0 | 0 | 100 | 0 | 0 |
| PROST2000162 | 0 | 0 | 0 | 0 | 100 | 0 | 0 |
| PROST2000165 | 0 | 0 | 0 | 0 | 32.383 | 0 | 0 |
| PROST2000176 | 0 | 0 | 0 | 0 | 9.001 | 0 | 0 |
| PROST2000203 | 0 | 0 | 0 | 0 | 100 | 0 | 0 |
| PROST2000206 | 0 | 0 | 0 | 0 | 77.659 | 0 | 0 |
| PROST2000212 | 0 | 0 | 0 | 0 | 100 | 0 | 0 |
| PROST2000241 | 0 | 0 | 0 | 0 | 21.702 | 0 | 0 |
| PROST2000267 | 0 | 0 | 0 | 0 | 100 | 0 | 0 |
| PROST2000273 | 0 | 0 | 0 | 0 | 74.833 | 0 | 0 |
| PROST2000274 | 0 | 0 | 0 | 18.366 | 36.598 | 0 | 0 |
| PROST2000277 | 0 | 0 | 0 | 0 | 75.996 | 0 | 0 |
| PROST2000284 | 0 | 0 | 0 | 0 | 12.419 | 0 | 0 |
| PROST2000325 | 0 | 53.208 | 0 | 0 | 25.104 | 0 | 0 |
| PROST2000337 | 0 | 0 | 0 | 0 | 65.749 | 0 | 0 |
| PROST2000432 | 0 | 0 | 0 | 0 | 48.885 | 0 | 0 |
| PROST2000452 | 0 | 0 | 0 | 0 | 100 | 0 | 0 |
| PROST2000463 | 0 | 0 | 0 | 0 | 60.719 | 0 | 0 |
| PROST2000470 | 0 | 0 | 0 | 0 | 100 | 0 | 0 |
| PROST2000505 | 0 | 0 | 0 | 0 | 100 | 0 | 0 |
| PROST2000567 | 0 | 0 | 0 | 0 | 50.797 | 0 | 0 |
| PROST2000572 | 0 | 0 | 0 | 0 | 100 | 0 | 0 |
| PROST2000717 | 0 | 0 | 0 | 0 | 100 | 0 | 0 |
| PROST2000760 | 0 | 0 | 0 | 0 | 9.948 | 0 | 0 |
| PROST2000761 | 0 | 0 | 0 | 0 | 33.566 | 0 | 0 |
| PROST2000835 | 0 | 0 | 0 | 0 | 75.996 | 0 | 0 |
| PROST2000893 | 0 | 0 | 0 | 0 | 58.722 | 0 | 0 |
| PROST2000961 | 0 | 0 | 0 | 0 | 100 | 0 | 0 |
| PROST2001002 | 0 | 0 | 0 | 0 | 69.674 | 0 | 0 |
| PROST2001116 | 0 | 0 | 0 | 0 | 75.996 | 0 | 0 |
| PROST2001138 | 0 | 0 | 0 | 0 | 100 | 0 | 0 |
| PROST2001180 | 0 | 0 | 0 | 0 | 8.805 | 0 | 0 |
| PROST2001213 | 0 | 0 | 0 | 0 | 59.127 | 0 | 0 |
| PROST2001283 | 0 | 0 | 0 | 0 | 7.546 | 0 | 0 |
| PROST2001414 | 0 | 0 | 0 | 0 | 100 | 0 | 0 |
| PROST2001415 | 0 | 0 | 0 | 0 | 100 | 0 | 0 |
| PROST2001465 | 0 | 0 | 0 | 0 | 100 | 0 | 0 |
| PROST2001521 | 0 | 0 | 0 | 0 | 4.309 | 0 | 52.873 |
| PROST2001540 | 0 | 0 | 0 | 0 | 100 | 0 | 0 |
| PROST2001552 | 0 | 0 | 0 | 0 | 3.087 | 0 | 0 |
| PROST2001599 | 0 | 0 | 0 | 0 | 100 | 0 | 0 |
| PROST2001611 | 0 | 0 | 0 | 0 | 100 | 0 | 0 |
| PROST2001636 | 0 | 0 | 0 | 0 | 100 | 0 | 0 |
| PROST2001739 | 0 | 0 | 0 | 0 | 11.723 | 0 | 0 |
| PROST2001796 | 0 | 0 | 0 | 0 | 100 | 0 | 0 |
| PROST2001805 | 0 | 0 | 0 | 0 | 100 | 0 | 0 |
| PROST2001823 | 0 | 0 | 0 | 0 | 65.749 | 0 | 0 |
| PROST2001883 | 0 | 0 | 0 | 0 | 3.167 | 0 | 0 |
| PROST2001899 | 0 | 0 | 0 | 0 | 100 | 0 | 0 |
| PROST2001997 | 0 | 0 | 0 | 0 | 20.676 | 0 | 0 |
| PROST2001998 | 0 | 0 | 0 | 0 | 100 | 0 | 0 |
| PROST2002078 | 0 | 0 | 0 | 0 | 8.618 | 0 | 0 |
| PROST2002101 | 0 | 0 | 0 | 0 | 100 | 0 | 0 |
| PROST2002162 | 0 | 0 | 0 | 0 | 13.729 | 0 | 0 |
| PROST2002212 | 0 | 0 | 0 | 0 | 100 | 0 | 0 |
| PROST2002227 | 0 | 0 | 0 | 0 | 9.014 | 0 | 0 |
| PROST2002338 | 0 | 0 | 0 | 0 | 100 | 0 | 0 |
| PROST2002425 | 0 | 0 | 0 | 0 | 56.349 | 0 | 0 |
| PROST2002488 | 0 | 0 | 0 | 0 | 100 | 0 | 0 |
| PROST2002489 | 0 | 0 | 0 | 0 | 100 | 0 | 0 |
| PROST2002527 | 0 | 0 | 0 | 0 | 100 | 0 | 0 |
| PROST2002591 | 0 | 0 | 0 | 0 | 100 | 0 | 0 |
| PROST2002602 | 0 | 0 | 0 | 0 | 100 | 0 | 0 |
| PROST2002633 | 0 | 0 | 0 | 0 | 100 | 0 | 0 |
| PROST2002651 | 0 | 0 | 0 | 0 | 10.605 | 0 | 0 |
| PROST2002682 | 0 | 0 | 0 | 0 | 100 | 0 | 0 |
| PROST2002685 | 0 | 0 | 0 | 0 | 100 | 0 | 0 |
| PROST2002722 | 0 | 0 | 0 | 0 | 100 | 0 | 0 |
| PROST2002736 | 0 | 0 | 0 | 0 | 100 | 0 | 0 |
| PROST2002842 | 0 | 0 | 0 | 0 | 100 | 0 | 0 |
| PROST2002897 | 0 | 0 | 0 | 0 | 75.996 | 0 | 0 |
| PROST2002906 | 0 | 0 | 0 | 0 | 100 | 0 | 0 |
| PROST2002927 | 0 | 0 | 0 | 0 | 37.967 | 0 | 0 |
| PROST2002960 | 0 | 0 | 0 | 0 | 47.668 | 0 | 0 |
| PROST2003102 | 0 | 0 | 0 | 0 | 100 | 0 | 0 |
| PROST2003117 | 0 | 0 | 0 | 0 | 100 | 0 | 0 |
| PROST2003210 | 0 | 0 | 0 | 0 | 100 | 0 | 0 |
| PROST2003232 | 0 | 0 | 0 | 1.214 | 4.839 | 0 | 0 |
| PROST2003302 | 0 | 0 | 0 | 0 | 100 | 0 | 0 |
| PROST2003303 | 0 | 0 | 0 | 0 | 100 | 0 | 0 |
| PROST2003324 | 0 | 0 | 0 | 0 | 62.709 | 0 | 0 |
| PROST2003338 | 0 | 0 | 0 | 0 | 3.969 | 6.316 | 0 |
| PROST2003340 | 0 | 0 | 0 | 0 | 100 | 0 | 0 |
| PROST2003396 | 0 | 0 | 0 | 21.264 | 21.187 | 0 | 0 |
| PROST2003428 | 0 | 0 | 0 | 0 | 67.090 | 0 | 0 |
| PROST2003472 | 0 | 0 | 0 | 0 | 100 | 0 | 0 |
| PROST2003517 | 0 | 0 | 0 | 0 | 18.673 | 0 | 0 |
| PROST2003537 | 0 | 0 | 0 | 0 | 75.996 | 0 | 0 |
| PROST2003563 | 0 | 0 | 0 | 0 | 100 | 0 | 0 |
| PROST2003577 | 0 | 0 | 0 | 0 | 100 | 0 | 0 |
| PROST2003583 | 0 | 0 | 0 | 0 | 100 | 0 | 0 |
| PROST2003586 | 0 | 0 | 0 | 0 | 100 | 0 | 0 |
| PROST2003628 | 0 | 0 | 0 | 0 | 100 | 0 | 0 |
| PROST2003635 | 0 | 0 | 0 | 0 | 100 | 0 | 0 |
| PROST2003674 | 0 | 0 | 0 | 0 | 100 | 0 | 0 |
| PROST2003676 | 0 | 0 | 0 | 0 | 100 | 0 | 0 |
| PROST2003725 | 0 | 0 | 0 | 0 | 80.963 | 0 | 0 |
| PROST2003732 | 0 | 0 | 0 | 0 | 100 | 0 | 0 |
| PROST2003775 | 0 | 0 | 0 | 0 | 100 | 0 | 0 |
| PROST2003922 | 0 | 0 | 0 | 0 | 100 | 0 | 0 |
| PROST2003930 | 0 | 0 | 0 | 0 | 100 | 0 | 0 |
| PROST2004095 | 0 | 0 | 0 | 0.833 | 4.980 | 0 | 0 |
| PROST2004115 | 0 | 0 | 0 | 0 | 100 | 0 | 0 |
| PROST2004142 | 0 | 0 | 0 | 0 | 100 | 0 | 0 |
| PROST2004146 | 0 | 0 | 0 | 0 | 100 | 0 | 0 |
| PROST2004161 | 0 | 0 | 0 | 0 | 100 | 0 | 0 |
| PROST2004251 | 0 | 0 | 0 | 0 | 3.177 | 0 | 0 |
| PROST2004258 | 0 | 0 | 0 | 0 | 27.061 | 0 | 0 |
| PROST2004270 | 0 | 0 | 0 | 0 | 10.427 | 0 | 0 |
| PROST2004332 | 0 | 0 | 0 | 0 | 100 | 0 | 0 |
| PROST2004416 | 0 | 0 | 0 | 1.210 | 2.411 | 0 | 0 |
| PROST2004481 | 0 | 0 | 0 | 0 | 100 | 0 | 0 |
| PROST2004526 | 0 | 0 | 0 | 0 | 100 | 0 | 0 |
| PROST2004570 | 0 | 0 | 0 | 0 | 26.582 | 0 | 0 |
| PROST2004727 | 0 | 0 | 0 | 0 | 100 | 0 | 0 |
| PROST2004739 | 0 | 0 | 0 | 0 | 26.371 | 0 | 0 |
| PROST2004742 | 0 | 0 | 0 | 0 | 100 | 0 | 0 |
| PROST2004744 | 0 | 39.737 | 0 | 0 | 18.748 | 0 | 0 |
| PROST2004817 | 0 | 0 | 0 | 0 | 100 | 0 | 0 |
| PROST2005039 | 0 | 0 | 0 | 0 | 48.872 | 0 | 0 |
| PROST2005067 | 0 | 0 | 0 | 0 | 57.209 | 0 | 0 |
| PROST2005076 | 0 | 0 | 48.502 | 0 | 25.535 | 0 | 0 |
| PROST2005093 | 0 | 0 | 0 | 0 | 83.324 | 0 | 0 |
| PROST2005121 | 0 | 0 | 0 | 0 | 100 | 0 | 0 |
| PROST2005131 | 0 | 0 | 0 | 0 | 100 | 0 | 0 |
| PROST2005143 | 0 | 0 | 0 | 0 | 24.058 | 0 | 0 |
| PROST2005228 | 0 | 0 | 0 | 0 | 100 | 0 | 0 |
| PROST2005272 | 0 | 0 | 0 | 0 | 29.123 | 0 | 0 |
| PROST2005285 | 0 | 0 | 0 | 13.283 | 26.470 | 0 | 0 |
| PROST2005426 | 0 | 0 | 0 | 0 | 100 | 0 | 0 |
| PROST2005466 | 0 | 0 | 0 | 0 | 83.324 | 0 | 0 |
| PROST2005604 | 0 | 0 | 0 | 0 | 11.031 | 0 | 0 |
| PROST2005630 | 0 | 0 | 0 | 0 | 100 | 0 | 0 |
| PROST2005683 | 0 | 0 | 61.281 | 0 | 32.263 | 0 | 0 |
| PROST2005788 | 0 | 0 | 0 | 0 | 100 | 0 | 0 |
| PROST2005793 | 0 | 0 | 0 | 0 | 13.907 | 0 | 0 |
| PROST2005880 | 0 | 0 | 0 | 0 | 100 | 0 | 0 |
| PROST2005886 | 0 | 0 | 0 | 0 | 100 | 0 | 0 |
| PROST2005904 | 0 | 0 | 0 | 0 | 100 | 0 | 0 |
| PROST2005919 | 0 | 0 | 0 | 0 | 100 | 0 | 0 |
| PROST2005943 | 0 | 0 | 0 | 0 | 100 | 0 | 0 |
| PROST2006008 | 0 | 0 | 0 | 0 | 100 | 0 | 0 |
| PROST2006020 | 0 | 0 | 0 | 0 | 100 | 0 | 0 |
| PROST2006030 | 0 | 0 | 0 | 6.734 | 13.418 | 0 | 0 |
| PROST2006060 | 0 | 0 | 0 | 0 | 23.679 | 0 | 0 |
| PROST2006196 | 0 | 0 | 0 | 0 | 100 | 0 | 0 |
| PROST2006201 | 0 | 0 | 0 | 0 | 100 | 0 | 0 |
| PROST2006260 | 0 | 0 | 0 | 7.247 | 14.441 | 0 | 0 |
| PROST2006282 | 0 | 0 | 0 | 0 | 31.502 | 0 | 0 |
| PROST2006343 | 0 | 0 | 0 | 0 | 31.360 | 0 | 0 |
| PROST2006450 | 0 | 0 | 0 | 0 | 100 | 0 | 0 |
| PROST2006491 | 0 | 0 | 0 | 0 | 100 | 0 | 0 |
| PROST2006510 | 0 | 0 | 0 | 0 | 100 | 0 | 0 |
| PROST2006536 | 0 | 0 | 0 | 0 | 23.609 | 0 | 0 |
| PROST2006579 | 0 | 0 | 0 | 0 | 100 | 0 | 0 |
| PROST2006688 | 0 | 0 | 0 | 0 | 100 | 0 | 0 |
| PROST2006737 | 0 | 0 | 0 | 0 | 8.595 | 0 | 0 |
| PROST2006766 | 0 | 0 | 0 | 0 | 100 | 0 | 0 |
| PROST2006782 | 0 | 0 | 0 | 0 | 100 | 0 | 0 |
| PROST2006933 | 0 | 0 | 0 | 0 | 67.090 | 0 | 0 |
| PROST2006988 | 0 | 0 | 0 | 0 | 51.786 | 0 | 0 |
| PROST2007122 | 0 | 0 | 0 | 0 | 100 | 0 | 0 |
| PROST2007200 | 0 | 0 | 0 | 0 | 41.890 | 0 | 0 |
| PROST2007237 | 0 | 0 | 0 | 0 | 100 | 0 | 0 |
| PROST2007248 | 0 | 0 | 0 | 0 | 66.101 | 0 | 0 |
| PROST2007289 | 0 | 0 | 0 | 0 | 100 | 0 | 0 |
| PROST2007317 | 0 | 0 | 0 | 0 | 84.436 | 0 | 0 |
| PROST2007328 | 0 | 0 | 0 | 0 | 100 | 0 | 0 |
| PROST2007382 | 0 | 0 | 0 | 0 | 100 | 0 | 0 |
| PROST2007389 | 0 | 0 | 0 | 0 | 100 | 0 | 0 |
| PROST2007416 | 0 | 0 | 0 | 0 | 100 | 0 | 0 |
| PROST2007528 | 0 | 0 | 0 | 0 | 100 | 0 | 0 |
| PROST2007612 | 0 | 0 | 0 | 0 | 11.325 | 0 | 0 |
| PROST2007818 | 0 | 0 | 0 | 0 | 100 | 0 | 0 |
| PROST2007871 | 0 | 0 | 0 | 0 | 39.115 | 0 | 0 |
| PROST2007950 | 0 | 0 | 0 | 0 | 100 | 0 | 0 |
| PROST2007956 | 0 | 0 | 0 | 0 | 100 | 0 | 0 |
| PROST2007974 | 0 | 0 | 0 | 0 | 100 | 0 | 0 |
| PROST2007988 | 0 | 0 | 0 | 0 | 100 | 0 | 0 |
| PROST2008079 | 0 | 0 | 0 | 0 | 100 | 0 | 0 |
| PROST2008088 | 0 | 0 | 0 | 0 | 100 | 0 | 0 |
| PROST2008243 | 0 | 0 | 0 | 0 | 100 | 0 | 0 |
| PROST2008245 | 0 | 0 | 0 | 0 | 100 | 0 | 0 |
| PROST2008268 | 0 | 0 | 0 | 0 | 65.749 | 0 | 0 |
| PROST2008271 | 0 | 0 | 0 | 0 | 13.898 | 0 | 0 |
| PROST2008360 | 0 | 0 | 0 | 0 | 12.766 | 0 | 0 |
| PROST2008447 | 0 | 0 | 0 | 0 | 100 | 0 | 0 |
| PROST2008468 | 0 | 0 | 0 | 0 | 100 | 0 | 0 |
| PROST2008472 | 0 | 0 | 0 | 0 | 100 | 0 | 0 |
| PROST2008489 | 0 | 0 | 0 | 0 | 2.297 | 7.309 | 0 |
| PROST2008516 | 0 | 0 | 0 | 0 | 100 | 0 | 0 |
| PROST2008724 | 0 | 0 | 0 | 0 | 100 | 0 | 0 |
| PROST2008770 | 0 | 0 | 0 | 0 | 2.766 | 0 | 0 |
| PROST2008835 | 0 | 0 | 0 | 0 | 100 | 0 | 0 |
| PROST2008993 | 0 | 0 | 0 | 0 | 100 | 0 | 0 |
| PROST2009022 | 0 | 0 | 0 | 0 | 100 | 0 | 0 |
| PROST2009208 | 0 | 0 | 0 | 0 | 100 | 0 | 0 |
| PROST2009222 | 0 | 0 | 0 | 0 | 100 | 0 | 0 |
| PROST2009254 | 0 | 0 | 0 | 0 | 100 | 0 | 0 |
| PROST2009273 | 0 | 0 | 0 | 0 | 100 | 0 | 0 |
| PROST2009320 | 0 | 0 | 0 | 0 | 50.124 | 0 | 0 |
| PROST2009347 | 0 | 0 | 0 | 0 | 67.212 | 0 | 0 |
| PROST2009365 | 0 | 0 | 0 | 0 | 100 | 0 | 0 |
| PROST2009388 | 0 | 0 | 0 | 0 | 100 | 0 | 0 |
| PROST2009400 | 0 | 0 | 0 | 21.223 | 42.291 | 0 | 0 |
| PROST2009470 | 0 | 0 | 0 | 0 | 100 | 0 | 0 |
| PROST2009483 | 0 | 0 | 0 | 0 | 100 | 0 | 0 |
| PROST2009571 | 0 | 0 | 0 | 0 | 100 | 0 | 0 |
| PROST2009731 | 0 | 0 | 0 | 0 | 3.846 | 0 | 0 |
| PROST2009736 | 0 | 0 | 0 | 0 | 100 | 0 | 0 |
| PROST2009795 | 0 | 0 | 0 | 0 | 100 | 0 | 0 |
| PROST2009901 | 0 | 0 | 0 | 0 | 100 | 0 | 0 |
| PROST2009909 | 0 | 0 | 0 | 0 | 100 | 0 | 0 |
| PROST2010046 | 0 | 0 | 0 | 0 | 100 | 0 | 0 |
| PROST2010219 | 0 | 0 | 0 | 0 | 75.996 | 0 | 0 |
| PROST2010250 | 0 | 0 | 0 | 0 | 100 | 0 | 0 |
| PROST2010318 | 0 | 0 | 0 | 0 | 85.605 | 0 | 0 |
| PROST2010326 | 0 | 0 | 0 | 0 | 100 | 0 | 0 |
| PROST2010382 | 0 | 0 | 0 | 0 | 11.098 | 0 | 0 |
| PROST2010400 | 0 | 0 | 0 | 0 | 100 | 0 | 0 |
| PROST2010545 | 0 | 0 | 0 | 0 | 46.670 | 0 | 0 |
| PROST2010579 | 0 | 0 | 0 | 0 | 66.659 | 0 | 0 |
| PROST2010606 | 0 | 0 | 0 | 0 | 100 | 0 | 0 |
| PROST2010782 | 0 | 0 | 0 | 0.866 | 2.587 | 0 | 0 |
| PROST2010885 | 0 | 0 | 0 | 0 | 100 | 0 | 0 |
| PROST2011012 | 0 | 0 | 0 | 0 | 100 | 0 | 0 |
| PROST2011038 | 0 | 0 | 0 | 0 | 42.556 | 0 | 0 |
| PROST2011105 | 0 | 0 | 0 | 0 | 63.107 | 0 | 0 |
| PROST2011297 | 0 | 0 | 0 | 0 | 100 | 0 | 0 |
| PROST2011410 | 0 | 0 | 0 | 0 | 14.062 | 0 | 0 |
| PROST2011439 | 0 | 0 | 0 | 0 | 100 | 0 | 0 |
| PROST2011482 | 0 | 0 | 0 | 0 | 65.749 | 0 | 0 |
| PROST2011577 | 0 | 0 | 0 | 0 | 3.649 | 0 | 0 |
| PROST2011631 | 0 | 0 | 0 | 0 | 100 | 0 | 0 |
| PROST2011660 | 0 | 0 | 0 | 0 | 83.324 | 0 | 0 |
| PROST2012005 | 0 | 0 | 0 | 0 | 100 | 0 | 0 |
| PROST2012007 | 0 | 0 | 0 | 2.020 | 4.025 | 0 | 0 |
| PROST2012016 | 0 | 0 | 0 | 0 | 77.874 | 0 | 0 |
| PROST2012088 | 0 | 0 | 0 | 0 | 100 | 0 | 0 |
| PROST2012140 | 0 | 0 | 0 | 0 | 65.565 | 0 | 0 |
| PROST2012157 | 0 | 0 | 0 | 0 | 100 | 0 | 0 |
| PROST2012190 | 0 | 0 | 0 | 0 | 100 | 0 | 0 |
| PROST2012249 | 0 | 0 | 0 | 0 | 100 | 0 | 0 |
| PROST2012353 | 0 | 0 | 0 | 0 | 33.336 | 0 | 0 |
| PROST2012400 | 0 | 0 | 0 | 0 | 100 | 0 | 0 |
| PROST2012448 | 0 | 0 | 0 | 0 | 28.935 | 0 | 0 |
| PROST2012504 | 0 | 0 | 0 | 0 | 80.963 | 0 | 0 |
| PROST2012542 | 0 | 0 | 0 | 0 | 100 | 0 | 0 |
| PROST2012550 | 0 | 0 | 0 | 0 | 100 | 0 | 0 |
| PROST2012740 | 0 | 0 | 0 | 0 | 100 | 0 | 0 |
| PROST2012745 | 0 | 0 | 0 | 0 | 13.715 | 0 | 0 |
| PROST2012780 | 0 | 0 | 0 | 0 | 25.798 | 0 | 0 |
| PROST2012888 | 0 | 0 | 0 | 0 | 59.538 | 0 | 0 |
| PROST2012890 | 0 | 0 | 0 | 0 | 54.091 | 0 | 0 |
| PROST2013032 | 0 | 0 | 0 | 0 | 100 | 0 | 0 |
| PROST2013053 | 0 | 0 | 0 | 5.271 | 10.504 | 0 | 0 |
| PROST2013121 | 0 | 0 | 0 | 19.142 | 38.145 | 0 | 0 |
| PROST2013260 | 0 | 0 | 0 | 0 | 100 | 0 | 0 |
| PROST2013327 | 0 | 0 | 0 | 0 | 100 | 0 | 0 |
| PROST2013531 | 0 | 0 | 0 | 0 | 100 | 0 | 0 |
| PROST2013605 | 0 | 0 | 0 | 0 | 100 | 0 | 0 |
| PROST2013627 | 0 | 0 | 0 | 0 | 100 | 0 | 0 |
| PROST2013873 | 0 | 0 | 0 | 0 | 100 | 0 | 0 |
| PROST2013880 | 0 | 0 | 0 | 0 | 100 | 0 | 0 |
| PROST2014422 | 0 | 0 | 0 | 0 | 100 | 0 | 0 |
| PROST2014585 | 0 | 0 | 0 | 0 | 100 | 0 | 0 |
| PROST2014601 | 0 | 0 | 0 | 0 | 100 | 0 | 0 |
| PROST2014659 | 0 | 0 | 0 | 0 | 100 | 0 | 0 |
| PROST2014862 | 0 | 0 | 0 | 0 | 56.349 | 0 | 0 |
| PROST2014916 | 0 | 0 | 0 | 0 | 12.920 | 0 | 0 |
| PROST2014925 | 0 | 0 | 0 | 0 | 100 | 0 | 0 |
| PROST2015124 | 0 | 0 | 0 | 0 | 100 | 0 | 0 |
| PROST2015137 | 0 | 0 | 0 | 0 | 65.163 | 0 | 0 |
| PROST2015198 | 0 | 0 | 0 | 0 | 100 | 0 | 0 |
| PROST2015243 | 0 | 0 | 0 | 0 | 100 | 0 | 0 |
| PROST2015246 | 0 | 0 | 0 | 0 | 100 | 0 | 0 |
| PROST2015251 | 0 | 0 | 0 | 0 | 76.179 | 0 | 0 |
| PROST2015287 | 0 | 0 | 0 | 0 | 38.071 | 0 | 0 |
| PROST2015332 | 0 | 0 | 17.362 | 0 | 9.141 | 0 | 0 |
| PROST2015457 | 0 | 0 | 0 | 0 | 100 | 0 | 0 |
| PROST2015464 | 0 | 0 | 0 | 0 | 6.083 | 0 | 37.318 |
| PROST2015537 | 0 | 0 | 0 | 0 | 100 | 0 | 0 |
| PROST2015545 | 0 | 0 | 0 | 0 | 100 | 0 | 0 |
| PROST2015551 | 0 | 0 | 0 | 0 | 100 | 0 | 0 |
| PROST2015636 | 0 | 0 | 0 | 0 | 100 | 0 | 0 |
| PROST2015710 | 0 | 0 | 0 | 0 | 43.675 | 0 | 0 |
| PROST2015756 | 0 | 0 | 0 | 0 | 100 | 0 | 0 |
| PROST2015877 | 0 | 0 | 0 | 0 | 100 | 0 | 0 |
| PROST2015900 | 0 | 0 | 0 | 0 | 83.324 | 0 | 0 |
| PROST2015924 | 0 | 50.935 | 0 | 0 | 24.031 | 0 | 0 |
| PROST2015932 | 0 | 0 | 0 | 0 | 100 | 0 | 0 |
| PROST2016195 | 0 | 0 | 0 | 0 | 20.739 | 0 | 0 |
| PROST2016246 | 0 | 0 | 0 | 0 | 100 | 0 | 0 |
| PROST2016351 | 0 | 0 | 59.664 | 0 | 31.411 | 0 | 0 |
| PROST2016379 | 0 | 0 | 0 | 0 | 100 | 0 | 0 |
| PROST2016444 | 0 | 0 | 0 | 0 | 100 | 0 | 0 |
| PROST2016462 | 0 | 0 | 0 | 0 | 100 | 0 | 0 |
| PROST2016499 | 0 | 0 | 0 | 0 | 100 | 0 | 0 |
| PROST2016512 | 0 | 0 | 0 | 0 | 100 | 0 | 0 |
| PROST2016566 | 0 | 0 | 0 | 0 | 100 | 0 | 0 |
| PROST2016582 | 0 | 0 | 65.511 | 0 | 34.489 | 0 | 0 |
| PROST2016668 | 0 | 0 | 0 | 0 | 100 | 0 | 0 |
| PROST2016684 | 0 | 0 | 0 | 0 | 100 | 0 | 0 |
| PROST2016777 | 0 | 0 | 0 | 0 | 100 | 0 | 0 |
| PROST2016829 | 0 | 0 | 0 | 0 | 100 | 0 | 0 |
| PROST2016860 | 0 | 0 | 0 | 0 | 100 | 0 | 0 |
| PROST2016918 | 0 | 0 | 0 | 0 | 22.538 | 0 | 0 |
| PROST2016980 | 0 | 0 | 0 | 0 | 80.304 | 0 | 0 |
| PROST2017098 | 0 | 0 | 0 | 0 | 100 | 0 | 0 |
| PROST2017128 | 0 | 0 | 54.279 | 0 | 28.576 | 0 | 0 |
| PROST2017203 | 0 | 0 | 0 | 0 | 4.605 | 0 | 0 |
| PROST2017367 | 0 | 0 | 0 | 0 | 100 | 0 | 0 |
| PROST2017413 | 0 | 0 | 0 | 0 | 100 | 0 | 0 |
| PROST2017441 | 0 | 0 | 0 | 0 | 100 | 0 | 0 |
| PROST2017529 | 0 | 0 | 0 | 0 | 9.001 | 0 | 0 |
| PROST2017578 | 0 | 0 | 0 | 0 | 100 | 0 | 0 |
| PROST2017612 | 0 | 0 | 0 | 0 | 75.996 | 0 | 0 |
| PROST2017617 | 0 | 0 | 0 | 0 | 74.833 | 0 | 0 |
| PROST2017692 | 0 | 0 | 0 | 0 | 2.613 | 0 | 0 |
| PROST2017700 | 0 | 0 | 0 | 0 | 100 | 0 | 0 |
| PROST2017701 | 0 | 0 | 0 | 0 | 100 | 0 | 0 |
| PROST2017729 | 0 | 0 | 0 | 0 | 50.124 | 0 | 0 |
| PROST2017$$36 | 0 | 0 | 0 | 0 | 100 | 0 | 0 |
| PROST2017910 | 0 | 0 | 0 | 0 | 37.058 | 0 | 0 |
| PROST2017972 | 0 | 0 | 0 | 0 | 42.891 | 0 | 0 |
| PROST2018030 | 0 | 0 | 0 | 0 | 100 | 0 | 0 |
| PROST2018090 | 0 | 0 | 0 | 0 | 7.715 | 0 | 47.331 |
| PROST2018235 | 0 | 0 | 0 | 0 | 100 | 0 | 0 |
| PROST2018437 | 0 | 0 | 0 | 0 | 100 | 0 | 0 |
| PROST2018487 | 0 | 0 | 0 | 0 | 100 | 0 | 0 |
| PROST2018511 | 0 | 0 | 0 | 0 | 100 | 0 | 0 |
| PROST2018583 | 0 | 0 | 0 | 0 | 32.777 | 0 | 0 |
| PROST2018588 | 0 | 0 | 0 | 23.355 | 46.539 | 0 | 0 |
| PROST2018607 | 0 | 0 | 0 | 0 | 59.538 | 0 | 0 |
| PROST2018788 | 0 | 0 | 0 | 0 | 100 | 0 | 0 |
| PROST2018902 | 0 | 0 | 0 | 0 | 100 | 0 | 0 |
| PROST2018922 | 0 | 0 | 0 | 0 | 100 | 0 | 0 |
| PROST2018977 | 0 | 0 | 0 | 0 | 46.084 | 0 | 0 |
| PROST2019164 | 0 | 0 | 0 | 0 | 13.077 | 0 | 0 |
| PROST2019253 | 0 | 0 | 0 | 0 | 5.958 | 0 | 0 |
| PROST2019296 | 0 | 0 | 0 | 0 | 20.168 | 0 | 0 |
| PROST2019398 | 0 | 0 | 0 | 0 | 100 | 0 | 0 |
| PROST2019487 | 0 | 0 | 0 | 0 | 100 | 0 | 0 |
| PROST2019781 | 0 | 0 | 0 | 0 | 100 | 0 | 0 |
| PUAEN1000039 | 0 | 0 | 0 | 0 | 0 | 100 | 0 |
| PUAEN1000057 | 0 | 0 | 0 | 0 | 0 | 23.169 | 0 |
| PUAEN1000065 | 0 | 0 | 13.535 | 0 | 16.627 | 3.779 | 0 |
| PUAEN1000081 | 0 | 0 | 0 | 0 | 0 | 75.183 | 0 |
| PUAEN1000085 | 0 | 0 | 0 | 0 | 0 | 100 | 0 |
| PUAEN1000087 | 0 | 0 | 0 | 0 | 0 | 100 | 0 |
| PUAEN1000121 | 0 | 0 | 0 | 0 | 0 | 100 | 0 |
| PUAEN1000147 | 0 | 0 | 0 | 0 | 0 | 52.615 | 0 |
| PUAEN1000161 | 0 | 0 | 0 | 20.531 | 0 | 32.548 | 0 |
| PUAEN1000164 | 0 | 0 | 0 | 0 | 0 | 4.492 | 0 |
| PUAEN1000175 | 0 | 0 | 0 | 0 | 0 | 100 | 0 |
| PUAEN1000239 | 0 | 0 | 0 | 0 | 0 | 17.492 | 0 |
| PUAEN1000275 | 0 | 0 | 0 | 0 | 0 | 100 | 0 |
| PUAEN1000276 | 0 | 0 | 0 | 0 | 0 | 100 | 0 |
| PUAEN1000288 | 0 | 0 | 0 | 0 | 0 | 100 | 0 |
| PUAEN1000322 | 0 | 0 | 0 | 0 | 0 | 7.213 | 0 |
| PUAEN2000080 | 0 | 0 | 0 | 0 | 0 | 100 | 0 |
| PUAEN2000152 | 0 | 0 | 0 | 0 | 0 | 66.977 | 0 |
| PUAEN2000175 | 0 | 0 | 0 | 0 | 0 | 19.914 | 0 |
| PUAEN2000247 | 0 | 0 | 0 | 0 | 19.593 | 62.349 | 0 |
| PUAEN2000312 | 0 | 0 | 0 | 0 | 0 | 49.955 | 0 |
| PUAEN2000374 | 0 | 0 | 0 | 1.709 | 0 | 5.418 | 0 |
| PUAEN2000394 | 0 | 0 | 0 | 0 | 0 | 100 | 0 |
| PUAEN2000497 | 0 | 0 | 0 | 0 | 0 | 14.547 | 0 |
| PUAEN2000535 | 0 | 6.045 | 0 | 0 | 0 | 4.538 | 0 |
| PUAEN2000594 | 0 | 0 | 0 | 0 | 0 | 60.344 | 0 |
| PUAEN2000684 | 0 | 0 | 0 | 0 | 0 | 100 | 0 |
| PUAEN2000942 | 0 | 0 | 0 | 0 | 0 | 75.335 | 0 |
| PUAEN2001260 | 0 | 0 | 0 | 0 | 0 | 100 | 0 |
| PUAEN2001345 | 0 | 0 | 0 | 0 | 0 | 16.521 | 0 |
| PUAEN2001526 | 0 | 0 | 0 | 0 | 0 | 100 | 0 |
| PUAEN2001586 | 0 | 0 | 0 | 5.082 | 0 | 16.112 | 0 |
| PUAEN2001882 | 0 | 0 | 0 | 0 | 0 | 61.957 | 0 |
| PUAEN2002473 | 0 | 0 | 0 | 0 | 0 | 100 | 0 |
| PUAEN2002489 | 0 | 0 | 0 | 0 | 0 | 36.817 | 0 |
| PUAEN2002552 | 0 | 0 | 0 | 0 | 0 | 100 | 0 |
| PUAEN2002568 | 0 | 0 | 0 | 0 | 0 | 61.524 | 0 |
| PUAEN2002616 | 0 | 0 | 0 | 0 | 0 | 100 | 0 |
| PUAEN2002758 | 0 | 0 | 0 | 0 | 0 | 100 | 0 |
| PUAEN2003018 | 0 | 0 | 0 | 0 | 0 | 18.914 | 0 |
| PUAEN2003079 | 0 | 0 | 0 | 0 | 0 | 100 | 0 |
| PUAEN2003116 | 0 | 0 | 0 | 0 | 0 | 100 | 0 |
| PUAEN2003408 | 0 | 0 | 0 | 0 | 0 | 16.993 | 0 |
| PUAEN2003517 | 0 | 0 | 0 | 0 | 0 | 100 | 0 |
| PUAEN2003954 | 0 | 0 | 0 | 0 | 0 | 61.526 | 0 |
| PUAEN2004067 | 0 | 0 | 0 | 0 | 0 | 100 | 0 |
| PUAEN2004083 | 0 | 0 | 0 | 0 | 0 | 100 | 0 |
| PUAEN2004400 | 0 | 0 | 0 | 0 | 0 | 40.652 | 0 |
| PUAEN2004511 | 0 | 0 | 0 | 0 | 0 | 100 | 0 |
| PUAEN2004525 | 0 | 0 | 0 | 0 | 0 | 100 | 0 |
| PUAEN2004875 | 0 | 0 | 0 | 0 | 0 | 100 | 0 |
| PUAEN2005110 | 0 | 0 | 0 | 0 | 0 | 100 | 0 |
| PUAEN2005247 | 0 | 0 | 0 | 0 | 0 | 100 | 0 |
| PUAEN2005502 | 0 | 0 | 0 | 0 | 0 | 60.392 | 0 |
| PUAEN2005588 | 0 | 0 | 0 | 0 | 0 | 100 | 0 |
| PUAEN2005930 | 0 | 0 | 0 | 0 | 0 | 51.805 | 0 |
| PUAEN2006029 | 0 | 0 | 0 | 0 | 0 | 100 | 0 |
| PUAEN2006328 | 0 | 0 | 0 | 0 | 0 | 20.807 | 0 |
| PUAEN2006335 | 0 | 0 | 0 | 0 | 0 | 71.562 | 0 |
| PUAEN2006578 | 0 | 0 | 0 | 0 | 0 | 100 | 0 |
| PUAEN2006639 | 0 | 0 | 0 | 0 | 0 | 100 | 0 |
| PUAEN2006761 | 0 | 0 | 0 | 0 | 0 | 100 | 0 |
| PUAEN2007044 | 0 | 0 | 0 | 5.102 | 0 | 8.088 | 0 |
| PUAEN2007350 | 0 | 0 | 0 | 0 | 0 | 75.640 | 0 |
| PUAEN2007785 | 0 | 0 | 0 | 0 | 0 | 100 | 0 |
| PUAEN2007898 | 0 | 0 | 0 | 0 | 0 | 60.962 | 0 |
| PUAEN2008123 | 0 | 0 | 0 | 0 | 0 | 100 | 0 |
| PUAEN2008228 | 0 | 0 | 0 | 0 | 0 | 82.551 | 0 |
| PUAEN2008314 | 0 | 0 | 0 | 0 | 0 | 60.344 | 0 |
| PUAEN2008515 | 0 | 0 | 0 | 0 | 0 | 54.781 | 0 |
| PUAEN2008815 | 0 | 0 | 0 | 0 | 0 | 70.071 | 0 |
| PUAEN2008939 | 0 | 0 | 0 | 6.108 | 0 | 77.460 | 0 |
| PUAEN2008980 | 0 | 0 | 0 | 0 | 0 | 100 | 0 |
| PUAEN2008985 | 0 | 0 | 0 | 0 | 0 | 25.036 | 0 |
| PUAEN2009174 | 0 | 0 | 0 | 0 | 0 | 31.987 | 0 |
| PUAEN2009348 | 0 | 0 | 0 | 0 | 0 | 44.559 | 0 |
| PUAEN2009534 | 0 | 0 | 0 | 0 | 0 | 100 | 0 |
| PUAEN2009655 | 0 | 0 | 0 | 0 | 0 | 20.623 | 0 |
| PUAEN2009795 | 0 | 0 | 0 | 0 | 0 | 100 | 0 |
| PUAEN2009852 | 0 | 0 | 0 | 0 | 0 | 100 | 0 |
| PUAEN2010346 | 0 | 0 | 0 | 0 | 0 | 56.319 | 0 |
| PUAEN2010824 | 0 | 0 | 0 | 0 | 0 | 29.560 | 0 |
| RECTM1000051 | 0 | 0 | 0 | 0 | 0 | 0 | 50.218 |
| RECTM1000141 | 0 | 0 | 0 | 0 | 0 | 0 | 100 |
| RECTM2000220 | 0 | 0 | 0 | 0 | 0 | 0 | 100 |
| RECTM2000349 | 0 | 0 | 0 | 0 | 0 | 0 | 92.174 |
| RECTM2000433 | 0 | 0 | 0 | 0 | 0 | 0 | 75.605 |
| RECTM2000510 | 0 | 0 | 0 | 0 | 0 | 0 | 65.580 |
| RECTM2001307 | 0 | 0 | 0 | 0 | 0 | 0 | 100 |
| RECTM2001347 | 0 | 0 | 0 | 0 | 0 | 0 | 90.247 |
| RECTM2001519 | 0 | 0 | 0 | 0 | 14.015 | 0 | 85.985 |
| RECTM2001666 | 0 | 0 | 0 | 0 | 0 | 0 | 100 |
| RECTM2001691 | 0 | 0 | 0 | 0 | 0 | 0 | 100 |
| RECTM2001749 | 0 | 0 | 0 | 0 | 0 | 0 | 100 |
| RECTM2002172 | 0 | 0 | 0 | 0 | 0 | 0 | 85.461 |
| RECTM2002602 | 0 | 0 | 0 | 0 | 0 | 0 | 100 |
| RECTM2002632 | 0 | 0 | 0 | 0 | 0 | 0 | 100 |
| SALGL1000005 | 0 | 0 | 0 | 0 | 1.075 | 0 | 0 |
| SALGL1000047 | 0 | 0 | 0 | 1.054 | 0 | 0 | 0 |
| SALGL1000157 | 0 | 0.977 | 0 | 0 | 0.922 | 0.734 | 0 |
| SKMUS1000022 | 0 | 0 | 2.804 | 0 | 0 | 0 | 0 |
| SKMUS1000064 | 0 | 0 | 15.807 | 0 | 0 | 0 | 0 |
| SKMUS1000084 | 0 | 0 | 0 | 2.741 | 0 | 0 | 0 |
| SKMUS1000138 | 0 | 0 | 0.647 | 0.171 | 0.341 | 0 | 0 |
| SKMUS1000186 | 0 | 3.017 | 2.704 | 2.143 | 0 | 0 | 0 |
| SKMUS2000361 | 0 | 0 | 0 | 0 | 0 | 12.068 | 0 |
| SKMUS2000380 | 0 | 0 | 0 | 0 | 7.691 | 0 | 0 |
| SKMUS2000458 | 0 | 0 | 6.438 | 0 | 0 | 0 | 0 |
| SKMUS2000515 | 0 | 0 | 0 | 20.164 | 0 | 0 | 0 |
| SKMUS2000679 | 0 | 0 | 22.564 | 0 | 0 | 0 | 0 |
| SKMUS2000873 | 0 | 0 | 18.526 | 0 | 1.951 | 0 | 0 |
| SKMUS2000931 | 0 | 8.180 | 3.665 | 1.937 | 0 | 0 | 0 |
| SKMUS2001008 | 0 | 0 | 3.192 | 0 | 0 | 0 | 0 |
| SKMUS2001129 | 0 | 0 | 34.255 | 9.050 | 0 | 0 | 0 |
| SKMUS2001147 | 0 | 0 | 0 | 0 | 0 | 15.641 | 0 |
| SKMUS2001157 | 0 | 0 | 24.165 | 0 | 0 | 0 | 0 |
| SKMUS2001201 | 0 | 0 | 47.998 | 0 | 0 | 0 | 0 |
| SKMUS2001402 | 0 | 0 | 56.794 | 0 | 0 | 0 | 0 |
| SKMUS2001454 | 0 | 0 | 2.273 | 1.201 | 0 | 0 | 0 |
| SKMUS2001634 | 0 | 0 | 0 | 4.660 | 0 | 0 | 0 |
| SKMUS2001668 | 0 | 0 | 19.961 | 0 | 0 | 0 | 0 |
| SKMUS2001693 | 0 | 0 | 32.341 | 0 | 0 | 0 | 0 |
| SKMUS2001695 | 0 | 0 | 36.451 | 0 | 0 | 0 | 0 |
| SKMUS2002634 | 0 | 0 | 57.706 | 0 | 6.076 | 0 | 0 |
| SKMUS2003168 | 0 | 0 | 0 | 0 | 33.480 | 0 | 0 |
| SKMUS2004667 | 0 | 0 | 0 | 0 | 33.480 | 0 | 0 |
| SKMUS2004903 | 0 | 0 | 48.875 | 0 | 0 | 0 | 0 |
| SKMUS2006755 | 0 | 0 | 48.875 | 0 | 0 | 0 | 0 |
| SKNMC1000137 | 0 | 0 | 0 | 15.092 | 0 | 0 | 0 |
| SKNMC1000168 | 0 | 0 | 0 | 0 | 8.909 | 0 | 18.220 |
| SKNMC2000097 | 0 | 3.350 | 0 | 0.793 | 0 | 0 | 0 |
| SKNMC2000322 | 0 | 0 | 0 | 2.513 | 0 | 0 | 0 |
| SKNMC2000635 | 0 | 0 | 0 | 1.277 | 1.908 | 2.024 | 0 |
| SKNMC2000649 | 0 | 0 | 0 | 6.495 | 0 | 0 | 0 |
| SKNMC2000698 | 0 | 0 | 0 | 0 | 7.325 | 11.655 | 0 |
| SKNMC2000877 | 0 | 0 | 0 | 0 | 0 | 0 | 25.782 |
| SKNMC2000966 | 0 | 0 | 0 | 0 | 28.717 | 0 | 0 |
| SKNMC2002402 | 0 | 0 | 0 | 13.120 | 0 | 0 | 0 |
| SKNSH1000002 | 0 | 0 | 0 | 2.558 | 5.097 | 0 | 0 |
| SKNSH1000101 | 0 | 4.828 | 0 | 0 | 0 | 0 | 0 |
| SKNSH2000347 | 0 | 0 | 0 | 7.986 | 0 | 0 | 0 |
| SKNSH2002768 | 0 | 0 | 0 | 8.427 | 8.397 | 13.360 | 0 |
| SKNSH2003528 | 0 | 0 | 0 | 5.403 | 0 | 17.130 | 0 |
| SKNSH2008940 | 0 | 0 | 0 | 0 | 0 | 10.820 | 0 |
| SMINT1000042 | 0 | 0 | 0 | 0 | 0 | 15.117 | 0 |
| SMINT1000078 | 0 | 0 | 0 | 0 | 66.777 | 0 | 0 |
| SMINT1000100 | 0 | 0 | 0 | 0 | 0 | 0 | 60.570 |
| SMINT1000117 | 0 | 9.886 | 0 | 0 | 0 | 0 | 0 |
| SMINT1000131 | 0 | 0 | 6.169 | 1.630 | 0 | 0 | 0 |
| SMINT2000018 | 0 | 0 | 0 | 0 | 14.931 | 0 | 0 |
| SMINT2000277 | 0 | 0 | 24.424 | 0 | 0 | 0 | 0 |
| SMINT2000396 | 0 | 0 | 0 | 13.071 | 0 | 6.907 | 0 |
| SMINT2000441 | 0 | 0 | 0 | 0.803 | 4.802 | 2.547 | 0 |
| SMINT2000468 | 0 | 0 | 0 | 4.072 | 0 | 0 | 0 |
| SMINT2000545 | 0 | 0 | 0 | 4.219 | 0 | 0 | 0 |
| SMINT2000602 | 0 | 8.263 | 0 | 0.978 | 0 | 0 | 0 |
| SMINT2000933 | 0 | 15.321 | 0 | 1.814 | 0 | 0 | 0 |
| SMINT2001818 | 0 | 7.046 | 0 | 0 | 3.324 | 0 | 0 |
| SMINT2002620 | 0 | 0 | 0 | 4.032 | 0 | 0 | 0 |
| SMINT2002689 | 0 | 0 | 0 | 4.184 | 0 | 0 | 0 |
| SMINT2002976 | 0 | 0 | 0 | 18.566 | 0 | 0 | 0 |
| SMINT2003505 | 0 | 23.670 | 0 | 0 | 0 | 0 | 0 |
| SMINT2004023 | 0 | 22.927 | 10.273 | 0 | 0 | 0 | 0 |
| SMINT2007433 | 0 | 0 | 0 | 14.856 | 0 | 0 | 0 |
| SMINT2008590 | 0 | 0 | 0 | 0 | 33.948 | 0 | 0 |
| SMINT2008917 | 0 | 0 | 0 | 0 | 39.679 | 0 | 0 |
| SMINT2008921 | 0 | 0 | 0 | 0 | 3.935 | 0 | 0 |
| SMINT2008960 | 0 | 0 | 4.608 | 0 | 0 | 0 | 0 |
| SMINT2009216 | 0 | 41.607 | 0 | 0 | 0 | 0 | 0 |
| SMINT2010084 | 0 | 0 | 0 | 6.115 | 0 | 0 | 0 |
| SMINT2010901 | 0 | 0 | 0 | 6.250 | 0 | 0 | 0 |
| SMINT2011311 | 0 | 0 | 0 | 33.526 | 0 | 0 | 0 |
| SMINT2011567 | 0 | 0 | 0 | 0 | 33.648 | 0 | 0 |
| SMINT2012122 | 0 | 0 | 23.549 | 0 | 0 | 0 | 0 |
| SMINT2013170 | 0 | 0 | 0 | 0 | 0 | 61.524 | 0 |
| SMINT2014480 | 0 | 11.942 | 0 | 0 | 0 | 0 | 0 |
| SMINT2018681 | 0 | 31.433 | 0 | 0 | 0 | 0 | 0 |
| SPLEN1000013 | 0 | 73.144 | 0 | 0 | 0 | 0 | 0 |
| SPLEN1000139 | 0 | 0 | 0 | 50.569 | 0 | 0 | 0 |
| SPLEN1000166 | 0 | 0 | 0 | 2.012 | 0 | 6.379 | 0 |
| SPLEN2000222 | 0 | 0 | 0 | 21.738 | 0 | 0 | 0 |
| SPLEN2000348 | 0 | 0 | 18.843 | 0 | 0 | 0 | 0 |
| SPLEN2000443 | 0 | 0 | 0 | 39.313 | 0 | 0 | 0 |
| SPLEN2000882 | 0 | 0 | 0 | 3.126 | 0 | 0 | 0 |
| SPLEN2001141 | 0 | 7.098 | 0 | 0 | 0 | 0 | 0 |
| SPLEN2001157 | 0 | 0 | 0 | 4.895 | 0 | 0 | 0 |
| SPLEN2002335 | 0 | 0 | 0 | 10.812 | 3.591 | 0 | 0 |
| SPLEN2002467 | 0 | 0 | 0 | 0 | 0 | 18.382 | 0 |
| SPLEN2002477 | 0 | 0 | 0 | 5.784 | 0 | 0 | 0 |
| SPLEN2002493 | 0 | 0 | 0 | 0 | 6.138 | 0 | 0 |
| SPLEN2002744 | 0 | 0 | 0 | 0 | 0 | 0 | 48.527 |
| SPLEN2005429 | 0 | 0 | 0 | 19.560 | 0 | 0 | 0 |
| SPLEN2006122 | 0 | 0 | 0 | 0 | 2.987 | 0 | 0 |
| SPLEN2006133 | 0 | 0 | 0 | 1.688 | 0 | 0 | 0 |
| SPLEN2006232 | 0 | 21.561 | 0 | 0 | 0 | 0 | 0 |
| SPLEN2006305 | 0 | 4.748 | 0 | 4.497 | 6.720 | 0 | 0 |
| SPLEN2007739 | 0 | 0 | 0 | 10.806 | 0 | 0 | 0 |
| SPLEN2007750 | 0 | 72.732 | 0 | 0 | 0 | 0 | 0 |
| SPLEN2007794 | 0 | 74.495 | 0 | 0 | 0 | 0 | 0 |
| SPLEN2008460 | 0 | 0 | 0 | 0 | 0 | 14.784 | 0 |
| SPLEN2010195 | 0 | 5.832 | 0 | 1.381 | 0 | 0 | 0 |
| SPLEN2010846 | 0 | 0 | 0 | 40.883 | 0 | 0 | 0 |
| SPLEN2011419 | 0 | 17.961 | 0 | 0 | 0 | 13.483 | 0 |
| SPLEN2011737 | 0 | 19.583 | 0 | 4.637 | 0 | 0 | 0 |
| SPLEN2011766 | 0 | 0 | 0 | 0 | 57.949 | 0 | 0 |
| SPLEN2012523 | 0 | 0 | 14.384 | 0 | 0 | 0 | 0 |
| SPLEN2012800 | 0 | 2.567 | 0 | 0 | 0 | 0 | 0 |
| SPLEN2014073 | 0 | 0 | 79.475 | 0 | 0 | 0 | 0 |
| SPLEN2014911 | 0 | 0 | 0 | 0 | 22.448 | 0 | 0 |
| SPLEN2015310 | 0 | 0 | 3.861 | 1.020 | 0 | 0 | 0 |
| SPLEN2016268 | 0 | 3.247 | 0 | 0 | 1.532 | 2.438 | 0 |
| SPLEN2016554 | 0 | 0 | 0 | 33.168 | 0 | 0 | 0 |
| SPLEN2017918 | 0 | 0 | 0 | 0 | 0 | 10.289 | 0 |
| SPLEN2018157 | 0 | 0 | 16.248 | 4.293 | 0 | 0 | 0 |
| SPLEN2019077 | 0 | 39.875 | 0 | 0 | 0 | 0 | 0 |
| SPLEN2023791 | 0 | 0 | 0 | 50.569 | 0 | 0 | 0 |
| SPLEN2027113 | 0 | 0 | 0 | 0 | 11.606 | 9.233 | 0 |
| SPLEN2028466 | 0 | 0 | 0 | 0 | 1.467 | 2.334 | 0 |
| SPLEN2028914 | 0 | 0 | 18.810 | 0 | 0 | 15.756 | 0 |
| SPLEN2029912 | 0 | 0 | 0 | 0 | 11.586 | 9.217 | 0 |
| SPLEN2031424 | 0 | 0 | 0 | 13.552 | 0 | 0 | 0 |
| SPLEN2033098 | 0 | 0 | 0 | 0 | 17.647 | 0 | 0 |
| SPLEN2036932 | 0 | 0 | 0 | 0 | 10.397 | 0 | 0 |
| SPLEN2037722 | 0 | 37.154 | 0 | 0 | 0 | 0 | 0 |
| SPLEN2038834 | 0 | 0 | 0 | 0 | 16.429 | 0 | 0 |
| SPLEN2042535 | 0 | 0 | 0 | 12.671 | 25.249 | 0 | 0 |
| SPLEN2042920 | 0 | 20.455 | 0 | 0 | 0 | 0 | 0 |
| STOMA1000189 | 0 | 0 | 0 | 2.783 | 0 | 0 | 0 |
| STOMA2000121 | 0 | 9.160 | 0 | 0 | 0 | 0 | 0 |
| STOMA2000257 | 0 | 0 | 0 | 0 | 7.000 | 0 | 0 |
| STOMA2000482 | 0 | 0 | 0 | 0 | 13.672 | 0 | 0 |
| STOMA2002166 | 0 | 0 | 0 | 0 | 2.075 | 0 | 0 |
| STOMA2004663 | 0 | 52.484 | 0 | 0 | 0 | 0 | 0 |
| SYNOV1000118 | 0 | 0 | 28.508 | 0 | 0 | 0 | 0 |
| SYNOV1000124 | 0 | 0 | 0 | 0 | 1.973 | 0 | 0 |
| SYNOV1000128 | 0 | 0 | 0 | 0 | 52.059 | 0 | 0 |
| SYNOV1000164 | 0 | 28.905 | 0 | 0 | 0 | 0 | 0 |
| SYNOV2001033 | 0 | 0 | 4.039 | 1.067 | 0 | 3.383 | 13.045 |
| SYNOV2001239 | 0 | 7.075 | 0 | 0 | 1.113 | 0 | 0 |
| SYNOV2001262 | 0 | 15.682 | 0 | 0 | 0 | 0 | 0 |
| SYNOV2001374 | 0 | 0 | 0 | 3.260 | 0 | 0 | 39.852 |
| SYNOV2001468 | 0 | 0 | 0 | 16.242 | 0 | 0 | 0 |
| SYNOV2009172 | 0 | 7.823 | 0 | 0 | 1.845 | 0 | 0 |
| SYNOV4001153 | 0 | 0 | 0 | 4.128 | 0 | 0 | 0 |
| SYNOV4007671 | 0 | 0 | 0 | 2.609 | 7.799 | 4.136 | 0 |
| SYNOV4009575 | 0 | 36.015 | 0 | 0 | 0 | 0 | 0 |
| T1ESE2002665 | 0 | 0 | 0 | 7.418 | 0 | 0 | 0 |
| TBAES2000116 | 0 | 0 | 0 | 20.191 | 0 | 0 | 0 |
| TBAES2009387 | 0 | 0 | 0 | 0.951 | 0 | 0 | 11.630 |
| TESOP1000035 | 0 | 33.245 | 0 | 0 | 0 | 0 | 0 |
| TESOP2002273 | 0 | 0 | 0 | 7.223 | 0 | 0 | 0 |
| TESOP2006053 | 0 | 0 | 49.610 | 0 | 0 | 0 | 0 |
| TESOP2008820 | 0 | 0 | 0 | 34.221 | 0 | 0 | 0 |
| TESTI1000023 | 0 | 0 | 5.649 | 0 | 0 | 0 | 0 |
| TESTI1000051 | 0 | 0 | 0 | 0 | 18.469 | 0 | 0 |
| TESTI1000064 | 0 | 0 | 0 | 0 | 17.264 | 0 | 0 |
| TESTI1000101 | 0 | 0 | 0 | 7.623 | 0 | 0 | 0 |
| TESTI1000142 | 0 | 0 | 0 | 0 | 0 | 38.072 | 0 |
| TESTI1000163 | 0 | 0 | 27.756 | 7.333 | 0 | 0 | 0 |
| TESTI2000006 | 0 | 74.325 | 0 | 0 | 0 | 0 | 0 |
| TESTI2000172 | 0 | 64.303 | 0 | 0 | 0 | 0 | 0 |
| TESTI2000184 | 0 | 8.497 | 0 | 4.024 | 0 | 0 | 0 |
| TESTI2000349 | 0 | 0 | 0 | 0 | 84.436 | 0 | 0 |
| TESTI2000435 | 0 | 0 | 0 | 0 | 68.199 | 0 | 0 |
| TESTI2000784 | 0 | 0 | 0 | 4.045 | 0 | 0 | 0 |
| TESTI2001099 | 0 | 0 | 3.301 | 1.744 | 0 | 2.765 | 0 |
| TESTI2001120 | 0 | 7.267 | 0 | 0 | 0 | 5.455 | 0 |
| TESTI2001153 | 0 | 0 | 32.187 | 0.709 | 0 | 0 | 0 |
| TESTI2001269 | 0 | 8.206 | 0 | 0 | 0 | 6.160 | 0 |
| TESTI2001829 | 0 | 0 | 0 | 1.511 | 6.023 | 0 | 0 |
| TESTI2002365 | 0 | 0 | 0 | 9.717 | 0 | 0 | 0 |
| TESTI2003005 | 0 | 0 | 0 | 0 | 10.774 | 0 | 0 |
| TESTI2003413 | 0 | 0 | 17.538 | 4.634 | 0 | 0 | 0 |
| TESTI2003533 | 0 | 0 | 5.840 | 0 | 3.075 | 0 | 0 |
| TESTI2003539 | 0 | 0 | 0 | 0 | 84.436 | 0 | 0 |
| TESTI2004031 | 0 | 0 | 0 | 1.427 | 0 | 0 | 0 |
| TESTI2004153 | 0 | 0 | 0 | 0 | 15.385 | 0 | 0 |
| TESTI2004431 | 0 | 0 | 0 | 0 | 29.665 | 0 | 0 |
| TESTI2004929 | 0 | 0 | 0 | 5.053 | 0 | 0 | 0 |
| TESTI2005408 | 0 | 13.317 | 0 | 0 | 6.283 | 0 | 0 |
| TESTI2005564 | 0 | 0 | 0 | 0 | 25.975 | 0 | 0 |
| TESTI2005784 | 0 | 0 | 0 | 5.293 | 0 | 0 | 0 |
| TESTI2005788 | 0 | 0 | 0 | 8.231 | 0 | 0 | 0 |
| TESTI2006051 | 0 | 0 | 8.557 | 0 | 0 | 0 | 0 |
| TESTI2006453 | 0 | 6.325 | 0 | 1.498 | 0 | 4.748 | 0 |
| TESTI2006588 | 0 | 0 | 0 | 2.890 | 3.840 | 0 | 0 |
| TESTI2006628 | 0 | 0 | 83.746 | 0 | 0 | 0 | 0 |
| TESTI2006633 | 0 | 0 | 0 | 47.576 | 0 | 0 | 0 |
| TESTI2006748 | 0 | 0 | 0 | 12.244 | 0 | 0 | 0 |
| TESTI2007407 | 0 | 0 | 0 | 0 | 0 | 17.320 | 0 |
| TESTI2007998 | 0 | 0 | 10.651 | 0 | 5.607 | 0 | 0 |
| TESTI2008240 | 0 | 0 | 1.684 | 18.246 | 1.774 | 0 | 0 |
| TESTI2008278 | 0 | 0 | 0 | 0 | 43.663 | 0 | 0 |
| TESTI2008621 | 0 | 0 | 0 | 17.980 | 0 | 0 | 0 |
| TESTI2009474 | 0 | 15.186 | 0 | 3.596 | 0 | 0 | 0 |
| TESTI2009520 | 0 | 0 | 0 | 4.004 | 0 | 0 | 0 |
| TESTI2009577 | 0 | 0 | 0 | 3.145 | 0 | 0 | 0 |
| TESTI2009785 | 0 | 0 | 0 | 0 | 5.088 | 0 | 0 |
| TESTI2010513 | 0 | 0 | 0 | 0 | 0 | 2.217 | 0 |
| TESTI2012155 | 0 | 48.166 | 0 | 0 | 0 | 0 | 0 |
| TESTI2012289 | 0 | 0 | 58.141 | 0 | 0 | 0 | 0 |
| TESTI2012778 | 0 | 2.778 | 0 | 0 | 0 | 1.043 | 0 |
| TESTI2013231 | 0 | 14.957 | 0 | 3.541 | 7.057 | 0 | 0 |
| TESTI2015092 | 0 | 0 | 22.316 | 0 | 0 | 0 | 0 |
| TESTI2015213 | 0 | 3.858 | 0 | 0 | 0 | 0 | 0 |
| TESTI2015626 | 0 | 2.472 | 0 | 0.585 | 0 | 0 | 0 |
| TESTI2016101 | 0 | 0 | 0 | 0 | 0 | 0 | 94.332 |
| TESTI2017954 | 0 | 0 | 0 | 0 | 2.349 | 0 | 0 |
| TESTI2018462 | 0 | 0 | 0 | 5.029 | 0 | 0 | 0 |
| TESTI2020510 | 0 | 0 | 0 | 0 | 0 | 40.816 | 0 |
| TESTI2020946 | 0 | 91.999 | 0 | 0 | 0 | 0 | 0 |
| TESTI2021297 | 0 | 0 | 0 | 0 | 0 | 38.736 | 0 |
| TESTI2021315 | 0 | 0 | 0 | 36.066 | 0 | 0 | 0 |
| TESTI2021425 | 0 | 0 | 0 | 6.128 | 0 | 0 | 0 |
| TESTI2023033 | 0 | 0 | 0 | 48.872 | 0 | 0 | 0 |
| TESTI2024090 | 0 | 0 | 0 | 73.137 | 0 | 0 | 0 |
| TESTI2024844 | 0 | 0 | 0 | 2.039 | 0 | 0 | 0 |
| TESTI2025546 | 0 | 0 | 0 | 12.354 | 0 | 19.584 | 0 |
| TESTI2026024 | 0 | 0 | 0 | 0 | 84.436 | 0 | 0 |
| TESTI2032643 | 0 | 0 | 81.912 | 0 | 0 | 0 | 0 |
| TESTI2033441 | 0 | 0 | 0 | 5.859 | 0 | 0 | 0 |
| TESTI2034249 | 0 | 0 | 0 | 6.955 | 13.858 | 0 | 0 |
| TESTI2034940 | 0 | 0 | 0 | 1.391 | 0.924 | 0 | 5.668 |
| TESTI2044276 | 0 | 0 | 0 | 16.878 | 0 | 0 | 0 |
| TESTI2046456 | 0 | 0 | 0 | 0.824 | 0 | 0 | 0 |
| TESTI2046721 | 0 | 91.999 | 0 | 0 | 0 | 0 | 0 |
| TESTI2053723 | 0 | 34.027 | 0 | 0 | 0 | 0 | 0 |
| TESTI4000014 | 0 | 2.685 | 0 | 0.318 | 0 | 0 | 0 |
| TESTI4001467 | 0 | 0 | 0 | 0 | 19.634 | 0 | 0 |
| TESTI4002072 | 0 | 0 | 0 | 5.943 | 0 | 0 | 0 |
| TESTI4002202 | 0 | 0 | 0 | 5.900 | 0 | 0 | 0 |
| TESTI4002799 | 0 | 0 | 2.975 | 0 | 0 | 0 | 0 |
| TESTI4009286 | 0 | 0 | 0 | 1.290 | 0 | 0 | 0 |
| TESTI4010851 | 0 | 0 | 3.683 | 2.919 | 0 | 0 | 0 |
| TESTI4013441 | 0 | 0 | 0 | 0 | 0.556 | 2.655 | 0 |
| TESTI4013474 | 0 | 15.533 | 0 | 0 | 0 | 0 | 0 |
| TESTI4018436 | 0 | 0 | 35.056 | 0 | 18.456 | 0 | 0 |
| TESTI4037156 | 0 | 2.080 | 0 | 0.492 | 0 | 0 | 0 |
| TESTI4038284 | 0 | 0 | 0 | 0 | 6.531 | 0 | 0 |
| TESTI4044704 | 0 | 38.311 | 0 | 0 | 0 | 0 | 0 |
| TESTI4046873 | 0 | 0 | 0 | 73.137 | 0 | 0 | 0 |
| THYMU1000002 | 0 | 0 | 7.974 | 1.053 | 2.099 | 3.340 | 0 |
| THYMU1000010 | 0 | 20.263 | 0 | 0 | 0 | 0 | 0 |
| THYMU1000016 | 0 | 0 | 0 | 0.688 | 0 | 2.181 | 0 |
| THYMU1000032 | 0 | 0 | 5.258 | 0 | 0 | 0 | 0 |
| THYMU1000083 | 0 | 0 | 0 | 0 | 10.634 | 0 | 0 |
| THYMU1000359 | 0 | 4.029 | 0 | 0 | 1.901 | 0 | 0 |
| THYMU1000394 | 0 | 0 | 0 | 8.749 | 17.433 | 0 | 0 |
| THYMU1000428 | 0 | 4.272 | 0 | 1.012 | 8.063 | 3.207 | 0 |
| THYMU2000057 | 0 | 20.389 | 0 | 0 | 0 | 0 | 0 |
| THYMU2000382 | 0 | 0 | 4.083 | 2.157 | 0 | 0 | 0 |
| THYMU2000684 | 0 | 0 | 0 | 7.410 | 14.765 | 0 | 0 |
| THYMU2001521 | 0 | 29.878 | 53.550 | 0 | 0 | 0 | 0 |
| THYMU2002229 | 0 | 81.842 | 0 | 0 | 0 | 0 | 0 |
| THYMU2002583 | 0 | 0 | 0 | 1.845 | 0 | 0 | 0 |
| THYMU2002745 | 0 | 0 | 0 | 0 | 0 | 0 | 83.299 |
| THYMU2002841 | 0 | 21.814 | 0 | 0 | 0 | 0 | 0 |
| THYMU2003012 | 0 | 6.587 | 0 | 0 | 0 | 0 | 0 |
| THYMU2003046 | 0 | 0 | 0 | 3.421 | 0 | 0 | 0 |
| THYMU2003069 | 0 | 0 | 0 | 7.866 | 0 | 0 | 0 |
| THYMU2003133 | 0 | 0 | 0 | 0 | 12.304 | 0 | 0 |
| THYMU2003419 | 0 | 0 | 0 | 4.469 | 1.781 | 0 | 0 |
| THYMU2003891 | 0 | 9.871 | 2.949 | 0 | 0.517 | 0 | 3.175 |
| THYMU2003981 | 0 | 3.926 | 0 | 0.929 | 0 | 0 | 0 |
| THYMU2004108 | 0 | 0 | 88.985 | 0 | 0 | 0 | 0 |
| THYMU2004152 | 0 | 14.926 | 0 | 0 | 0 | 0 | 0 |
| THYMU2004320 | 0 | 0 | 0 | 0 | 57.949 | 0 | 0 |
| THYMU2004344 | 0 | 0 | 0 | 2.273 | 4.530 | 0 | 0 |
| THYMU2004410 | 0 | 0 | 0 | 3.410 | 0 | 10.811 | 0 |
| THYMU2004733 | 0 | 0 | 37.726 | 0 | 0 | 0 | 0 |
| THYMU2005156 | 0 | 0 | 0 | 68.095 | 0 | 0 | 0 |
| THYMU2005546 | 0 | 0 | 0 | 0 | 0 | 3.086 | 0 |
| THYMU2005980 | 0 | 0 | 0 | 14.432 | 0 | 0 | 0 |
| THYMU2006505 | 0 | 0 | 0 | 2.429 | 0 | 0 | 0 |
| THYMU2006813 | 0 | 0 | 0 | 2.816 | 5.612 | 0 | 0 |
| THYMU2007036 | 0 | 58.036 | 0 | 0 | 0 | 0 | 0 |
| THYMU2007065 | 0 | 0 | 27.315 | 0 | 0 | 0 | 0 |
| THYMU2007112 | 0 | 0 | 0 | 0 | 16.219 | 0 | 0 |
| THYMU2007886 | 0 | 10.408 | 0 | 0 | 0 | 7.813 | 0 |
| THYMU2008725 | 0 | 0 | 0 | 0 | 0 | 73.576 | 0 |
| THYMU2009658 | 0 | 0 | 2.360 | 0 | 0 | 0 | 0 |
| THYMU2009792 | 0 | 0 | 0 | 0 | 0 | 15.803 | 0 |
| THYMU2010094 | 0 | 43.781 | 0 | 0 | 0 | 0 | 0 |
| THYMU2011881 | 0 | 78.357 | 0 | 0 | 0 | 0 | 0 |
| THYMU2012024 | 0 | 44.799 | 0 | 5.303 | 0 | 16.815 | 0 |
| THYMU2012625 | 0 | 0 | 0 | 0 | 0 | 0 | 22.602 |
| THYMU2013136 | 0 | 0 | 0 | 0 | 2.901 | 0 | 0 |
| THYMU2013648 | 0 | 90.014 | 0 | 0 | 0 | 0 | 0 |
| THYMU2013916 | 0 | 0 | 0 | 0 | 3.933 | 0 | 0 |
| THYMU2014167 | 0 | 0 | 0 | 0 | 19.488 | 0 | 0 |
| THYMU2014183 | 0 | 73.025 | 0 | 0 | 0 | 0 | 0 |
| THYMU2015019 | 0 | 0 | 0 | 0 | 0 | 77.187 | 0 |
| THYMU2018126 | 0 | 5.468 | 0 | 0 | 0 | 0 | 0 |
| THYMU2019021 | 0 | 0 | 32.736 | 0 | 0 | 0 | 0 |
| THYMU2025707 | 0 | 0 | 62.325 | 0 | 0 | 0 | 0 |
| THYMU2027125 | 0 | 0 | 0 | 1.274 | 0.635 | 0 | 3.895 |
| THYMU2027975 | 0 | 0 | 0 | 7.113 | 0 | 0 | 0 |
| THYMU2028629 | 0 | 0 | 0 | 32.582 | 0 | 0 | 0 |
| THYMU2031341 | 0 | 0 | 0 | 0 | 12.236 | 0 | 0 |
| THYMU2035735 | 0 | 0 | 0 | 0 | 4.678 | 0 | 28.704 |
| THYMU2036461 | 0 | 0 | 0 | 0 | 53.789 | 0 | 0 |
| THYMU3009006 | 0 | 0 | 0 | 33.017 | 0 | 0 | 0 |
| THYMU3013386 | 0 | 0 | 0 | 0 | 2.655 | 0 | 0 |
| THYMU3015759 | 0 | 8.463 | 0 | 0 | 3.993 | 6.353 | 0 |
| THYMU3024339 | 0 | 0 | 0 | 51.624 | 0 | 0 | 0 |
| THYMU3025642 | 0 | 0 | 0 | 68.095 | 0 | 0 | 0 |
| THYMU3034671 | 0 | 0 | 0 | 21.222 | 0 | 0 | 0 |
| THYMU3038158 | 0 | 0 | 41.483 | 0 | 0 | 0 | 0 |
| THYMU3047115 | 0 | 0 | 19.376 | 0 | 1.457 | 0 | 0 |
| TKIDN1000066 | 0 | 0 | 64.536 | 0 | 0 | 0 | 0 |
| TKIDN1000164 | 0 | 46.474 | 0 | 0 | 0 | 0 | 0 |
| TKIDN2001662 | 0 | 0 | 0 | 19.409 | 0 | 0 | 0 |
| TKIDN2002738 | 0 | 3.152 | 2.825 | 0 | 0 | 0 | 0 |
| TKIDN2003597 | 0 | 0 | 0 | 13.903 | 0 | 0 | 0 |
| TKIDN2007828 | 0 | 0 | 44.943 | 0 | 0 | 0 | 0 |
| TKIDN2015285 | 0 | 0 | 0 | 0 | 35.207 | 0 | 0 |
| TKIDN2015423 | 0 | 0 | 0 | 2.266 | 0 | 0 | 0 |
| TKIDN2015788 | 0 | 0 | 0 | 4.681 | 0 | 0 | 0 |
| TLIVE2002046 | 0 | 0 | 0 | 6.460 | 6.437 | 0 | 0 |
| TLIVE2003970 | 0 | 0 | 0 | 0 | 0 | 13.050 | 0 |
| TRACH1000030 | 0 | 0 | 0 | 3.683 | 4.893 | 15.571 | 0 |
| TRACH1000038 | 0 | 0 | 25.370 | 0 | 0 | 0 | 0 |
| TRACH1000063 | 0 | 16.166 | 0 | 0 | 0 | 0 | 0 |
| TRACH1000106 | 0 | 0 | 39.991 | 0 | 0 | 0 | 0 |
| TRACH1000205 | 0 | 0 | 0 | 0 | 0 | 22.206 | 0 |
| TRACH2000079 | 0 | 4.072 | 7.298 | 0 | 1.921 | 6.113 | 0 |
| TRACH2000081 | 0 | 0 | 85.742 | 0 | 0 | 0 | 0 |
| TRACH2000248 | 0 | 10.488 | 4.700 | 0 | 0 | 3.937 | 0 |
| TRACH2000321 | 0 | 0 | 3.310 | 1.749 | 0 | 0 | 0 |
| TRACH2000420 | 0 | 0 | 0 | 0 | 35.676 | 0 | 0 |
| TRACH2000472 | 0 | 0 | 0 | 0 | 6.056 | 0 | 37.157 |
| TRACH2000702 | 0 | 29.510 | 0 | 0 | 0 | 0 | 0 |
| TRACH2000959 | 0 | 0 | 0 | 0 | 0.866 | 0 | 0 |
| TRACH2000972 | 0 | 8.909 | 0 | 0 | 0 | 0 | 0 |
| TRACH2001021 | 0 | 0 | 5.641 | 1.490 | 1.485 | 0 | 9.110 |
| TRACH2001101 | 0 | 0 | 0 | 0 | 9.426 | 0 | 0 |
| TRACH2001289 | 0 | 5.110 | 2.290 | 1.210 | 2.411 | 0 | 0 |
| TRACH2001314 | 0 | 0 | 0 | 3.253 | 6.482 | 0 | 0 |
| TRACH2001443 | 0 | 0 | 0 | 16.537 | 0 | 0 | 0 |
| TRACH2001463 | 0 | 0 | 0 | 0 | 10.858 | 0 | 0 |
| TRACH2001478 | 0 | 0 | 3.910 | 0 | 0 | 3.276 | 0 |
| TRACH2001523 | 0 | 0 | 0 | 0 | 6.029 | 0 | 0 |
| TRACH2001607 | 0 | 0 | 0 | 1.787 | 3.562 | 0 | 0 |
| TRACH2001612 | 0 | 0 | 0 | 7.109 | 0 | 0 | 0 |
| TRACH2001898 | 0 | 0 | 0 | 0 | 3.615 | 0 | 0 |
| TRACH2002092 | 0 | 21.099 | 18.909 | 0 | 0 | 0 | 0 |
| TRACH2002537 | 0 | 0 | 0 | 0 | 3.289 | 5.233 | 0 |
| TRACH2002664 | 0 | 0 | 10.251 | 0 | 10.794 | 0 | 0 |
| TRACH2002847 | 0 | 0 | 0 | 0 | 0 | 25.610 | 0 |
| TRACH2003323 | 0 | 13.377 | 0 | 0 | 0 | 0 | 0 |
| TRACH2004499 | 0 | 0 | 19.794 | 0 | 0 | 0 | 0 |
| TRACH2005811 | 0 | 0 | 0 | 0 | 3.740 | 0 | 0 |
| TRACH2006762 | 0 | 0 | 0 | 0 | 75.996 | 0 | 0 |
| TRACH2007399 | 0 | 0 | 3.173 | 0 | 0 | 0 | 0 |
| TRACH2007754 | 0 | 0 | 0 | 2.674 | 0 | 0 | 32.689 |
| TRACH2007816 | 0 | 0 | 0 | 17.766 | 0 | 0 | 0 |
| TRACH2008472 | 0 | 0 | 10.569 | 0 | 0 | 0 | 0 |
| TRACH2008891 | 0 | 0 | 0 | 38.608 | 0 | 0 | 0 |
| TRACH2009006 | 0 | 52.758 | 0 | 0 | 0 | 0 | 0 |
| TRACH2010600 | 0 | 0 | 0 | 61.372 | 0 | 0 | 0 |
| TRACH2011500 | 0 | 0 | 0 | 0 | 47.884 | 0 | 0 |
| TRACH2012536 | 0 | 0 | 0 | 61.372 | 0 | 0 | 0 |
| TRACH2013991 | 0 | 0 | 85.742 | 0 | 0 | 0 | 0 |
| TRACH2014124 | 0 | 0 | 0 | 3.674 | 3.661 | 0 | 0 |
| TRACH2015823 | 0 | 0 | 0 | 3.893 | 0 | 0 | 0 |
| TRACH2015824 | 0 | 0 | 0 | 4.136 | 0 | 0 | 0 |
| TRACH2015939 | 0 | 42.223 | 0 | 0 | 0 | 0 | 0 |
| TRACH2017368 | 0 | 0 | 0 | 0 | 0 | 0 | 95.104 |
| TRACH2018317 | 0 | 0 | 0 | 0 | 0 | 16.530 | 0 |
| TRACH2018446 | 0 | 0 | 0 | 14.886 | 0 | 0 | 0 |
| TRACH3000692 | 0 | 0 | 0 | 8.341 | 0 | 26.446 | 0 |
| TRACH3002192 | 0 | 0 | 0 | 0 | 2.212 | 0 | 0 |
| TRACH3003547 | 0 | 0 | 0 | 0 | 14.997 | 0 | 0 |
| TRACH3004068 | 0 | 0 | 0 | 2.537 | 0 | 0 | 31.010 |
| TRACH3005699 | 0 | 0 | 0 | 17.081 | 0 | 0 | 0 |
| TRACH3006717 | 0 | 0 | 0 | 13.061 | 0 | 0 | 0 |
| TRACH3007625 | 0 | 0 | 7.918 | 0 | 4.169 | 0 | 0 |
| TRACH3008632 | 0 | 0 | 0 | 15.039 | 0 | 0 | 0 |
| TRACH3009148 | 0 | 0 | 0 | 31.925 | 0 | 0 | 0 |
| TRACH3016805 | 0 | 0 | 0 | 8.013 | 0 | 0 | 0 |
| TRACH3037067 | 0 | 48.330 | 0 | 0 | 0 | 0 | 0 |
| TSTOM2000139 | 0 | 0 | 0 | 7.443 | 0 | 0 | 0 |
| TSTOM2000315 | 0 | 17.130 | 0 | 0 | 0 | 0 | 0 |
| TSTOM2000553 | 0 | 0 | 0 | 3.000 | 0 | 0 | 0 |
| TUTER2000904 | 0 | 0 | 0 | 4.968 | 0 | 0 | 0 |
| UMVEN1000186 | 0 | 0 | 0 | 0.940 | 0 | 11.927 | 0 |
| UMVEN2000453 | 0 | 3.925 | 3.517 | 0.929 | 5.555 | 0 | 0 |
| UTERU1000182 | 0 | 0 | 0 | 2.887 | 2.877 | 0 | 0 |
| UTERU1000384 | 0 | 0 | 0 | 12.684 | 0 | 0 | 0 |
| UTERU2000095 | 0 | 0 | 5.576 | 0.737 | 0 | 4.671 | 0 |
| UTERU2000263 | 0 | 0 | 0 | 0 | 0 | 2.891 | 0 |
| UTERU2000300 | 0 | 0 | 0 | 0 | 0 | 27.831 | 0 |
| UTERU2000338 | 0 | 0 | 0 | 0 | 11.831 | 0 | 72.584 |
| UTERU2000424 | 0 | 6.384 | 0 | 0 | 6.024 | 0 | 0 |
| UTERU2000537 | 0 | 38.565 | 0 | 0 | 0 | 0 | 0 |
| UTERU2000542 | 0 | 0 | 60.821 | 0 | 0 | 0 | 0 |
| UTERU2000696 | 0 | 0 | 0 | 5.064 | 0 | 0 | 0 |
| UTERU2001607 | 0 | 0 | 0 | 2.625 | 0 | 0 | 0 |
| UTERU2001658 | 0 | 0 | 0 | 0 | 31.413 | 0 | 0 |
| UTERU2002001 | 0 | 0 | 0 | 59.874 | 0 | 0 | 0 |
| UTERU2002410 | 0 | 0 | 0 | 2.090 | 2.082 | 0 | 0 |
| UTERU2003704 | 0 | 0 | 0 | 30.632 | 0 | 0 | 0 |
| UTERU2003926 | 0 | 0 | 0 | 59.874 | 0 | 0 | 0 |
| UTERU2004037 | 0 | 16.480 | 0 | 0 | 0 | 0 | 0 |
| UTERU2004197 | 0 | 0 | 0 | 3.579 | 0 | 11.346 | 0 |
| UTERU2004807 | 0 | 0 | 0 | 8.939 | 7.125 | 0 | 0 |
| UTERU2004929 | 0 | 0 | 0 | 0 | 41.168 | 0 | 0 |
| UTERU2005354 | 0 | 16.442 | 0 | 3.893 | 0 | 0 | 0 |
| UTERU2005664 | 0 | 0 | 75.272 | 0 | 0 | 0 | 0 |
| UTERU2006593 | 0 | 0 | 0 | 5.255 | 5.236 | 0 | 0 |
| UTERU2007081 | 0 | 0 | 0 | 0 | 0 | 82.551 | 0 |
| UTERU2008040 | 0 | 0 | 0 | 0 | 41.737 | 0 | 0 |
| UTERU2008085 | 0 | 0.679 | 6.083 | 0.161 | 0.320 | 0.510 | 1.965 |
| UTERU2008302 | 0 | 10.042 | 0 | 0 | 0 | 0 | 0 |
| UTERU2008938 | 0 | 0 | 0 | 8.760 | 0 | 0 | 0 |
| UTERU2008939 | 0 | 0 | 0 | 26.435 | 0 | 0 | 0 |
| UTERU2010417 | 0 | 0 | 0 | 1.122 | 0 | 1.779 | 0 |
| UTERU2011220 | 0 | 0 | 0 | 3.483 | 0 | 0 | 0 |
| UTERU2012286 | 0 | 19.044 | 0 | 0 | 0 | 0 | 0 |
| UTERU2012856 | 0 | 0 | 0 | 6.279 | 0 | 0 | 0 |
| UTERU2013502 | 0 | 0 | 0 | 0 | 59.786 | 0 | 0 |
| UTERU2014167 | 0 | 0 | 0 | 0 | 3.232 | 0 | 0 |
| UTERU2014898 | 0 | 0 | 0 | 2.708 | 0 | 0 | 0 |
| UTERU2016822 | 0 | 0 | 0 | 14.308 | 0 | 11.341 | 0 |
| UTERU2016979 | 0 | 0 | 69.671 | 0 | 0 | 0 | 0 |
| UTERU2017492 | 0 | 0 | 0 | 0 | 0 | 0 | 94.803 |
| UTERU2017613 | 0 | 0 | 0 | 0 | 36.467 | 0 | 0 |
| UTERU2018523 | 0 | 0 | 0 | 59.874 | 0 | 0 | 0 |
| UTERU2018884 | 0 | 0 | 0 | 2.757 | 0 | 8.741 | 0 |
| UTERU2019005 | 0 | 0 | 0 | 0 | 22.904 | 0 | 0 |
| UTERU2019257 | 0 | 0 | 0 | 42.729 | 0 | 0 | 0 |
| UTERU2023175 | 0 | 0 | 0 | 5.299 | 0 | 0 | 0 |
| UTERU2023262 | 0 | 0 | 84.958 | 0 | 0 | 0 | 0 |
| UTERU2024969 | 0 | 24.138 | 0 | 0 | 0 | 0 | 0 |
| UTERU3004477 | 0 | 0 | 0 | 42.729 | 0 | 0 | 0 |
| UTERU3005907 | 0 | 0 | 0 | 69.877 | 0 | 0 | 0 |
| UTERU3010604 | 0 | 0 | 0 | 59.874 | 0 | 0 | 0 |
| UTERU3018616 | 0 | 0 | 0 | 0 | 74.833 | 0 | 0 |

Genes whose expression levels were negligibly low in each of the above-described libraries are shown with their clone names below.

3NB691000018, 3NB691000113, 3NB691000191, 3NB692000012, 3NB692000029, 3NB692000102, 3NB692000154, 3NB692000276, 3NB692000305, 3NB692000429, 3NB692000484, 3NB692000529, 3NB692000973, 3NB692001034, 3NB692001040, 3NB692001123, 3NB692001267, 3NB692001288, 3NB692001339, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001520, 3NB692001548, 3NB692001637, 3NB692001719, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064, ADIPS2000069,

ADIPS2000088, ADIPS2000245, ADRGL1000002, ADRGL1000018, ADRGL1000033, ADRGL1000038, ADRGL1000065, ADRGL1000067, ADRGL1000111, ADRGL1000147, ADRGL1000160, ADRGL1000165, ADRGL1000182, ADRGL2000042, ADRGL2000056, ADRGL2000064, ADRGL2000074, ADRGL2000085, ADRGL2000097, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002477, ADRGL2002679,

ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023,

ADRGL2008331, ADRGL2008337, ADRGL2008535, ADRGL2008966, ADRGL2009146, ADRGL2009273, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO2000011, ASTRO2000095, ASTRO2000191, ASTRO2000278, ASTRO2000372, ASTRO2000381, ASTRO2000403, ASTRO2000417, ASTRO2000482, ASTRO2000533, ASTRO2000653, ASTRO2000662, ASTRO2000725, ASTRO2000801, ASTRO2001001, ASTRO2001008, ASTRO2001076, ASTRO2001088, ASTRO2001107, ASTRO2001122, ASTRO2001227, ASTRO2001249, ASTRO2001480, ASTRO2002024, ASTRO2002035,

ASTRO2002064, ASTRO2002202, ASTRO2002459, ASTRO2002571, ASTRO2002632, ASTRO2002659, ASTRO2002693, ASTRO2002720, ASTRO2002733, ASTRO2002743, ASTRO2002842, ASTRO2003024, ASTRO2003212, ASTRO2003316, ASTRO2003461, ASTRO2003574, ASTRO2003581, ASTRO2003632, ASTRO2003740, ASTRO2003840, ASTRO2003925, ASTRO2003960, ASTRO2004051, ASTRO2004177, ASTRO2004217, ASTRO2004297, ASTRO2004584, ASTRO2004751, ASTRO2004877, ASTRO2004974, ASTRO2005008, ASTRO2005081, ASTRO2005096, ASTRO2005273, ASTRO2005343, ASTRO2005360, ASTRO2005413, ASTRO2005423, ASTRO2005553, ASTRO2005557, ASTRO2005575, ASTRO2005593, ASTRO2005726, ASTRO2005896, ASTRO2005902, ASTRO2006356, ASTRO2006732, ASTRO2006747, ASTRO2006952, ASTRO2007047,

ASTRO2007221, ASTRO2007377, ASTRO2007515, ASTRO2007578, ASTRO2007643, ASTRO2007666, ASTRO2007956, ASTRO2007962, ASTRO2008040, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561, ASTRO2014576, ASTRO2014863, ASTRO2014923,

ASTRO2014961, ASTRO2015162, ASTRO2015185, ASTRO2015328, ASTRO2015529, ASTRO2016044, ASTRO2016148, ASTRO2016681, ASTRO2016819, ASTRO2016847, ASTRO2016892, ASTRO2017348, ASTRO2017627, ASTRO2018169, ASTRO2018373, ASTRO2019039, ASTRO2019171, ASTRO3000154, ASTRO3000172, ASTRO3000177, ASTRO3000301, ASTRO3000474, ASTRO3000482, BEAST2000454, BGGI11000123, BGGI11000193, BGGI11000285, BGGI12000022, BGGI12000067, BGGI12000161, BGGI12000544, BGGI12000684, BGGI12000693, BGGI12001002, BGGI12001075, BGGI12001241, BGGI12001247, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031,

BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006830, BLADE2006849, BLADE2006947, BLADE2007033, BLADE2007043, BLADE2007666, BLADE2007722, BLADE2007735, BLADE2007744, BLADE2007799, BLADE2007923, BLADE2007935, BLADE2007958,

BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000118, BNGH41000137, BNGH41000169, BNGH41000177, BNGH41000198, BNGH41000216, BNGH41000235, BNGH42000130, BNGH42000132, BNGH42000405, BNGH42000532, BNGH42000675, BNGH42000791, BNGH42000815, BNGH42000994, BNGH42001247, BNGH42001406, BNGH42001576, BNGH42001724, BNGH42002168, BNGH42002244, BNGH42002387, BNGH42002489, BNGH42003529, BNGH42003570, BNGH42003641, BNGH42004076, BNGH42004291, BNGH42004538, BNGH42004679, BNGH42005017, BNGH42005235, BNGH42005504, BNGH42006226, BNGH42006234, BNGH42007037, BNGH42007366, BNGH42007594, BNGH42007798,

BNGH42008199, BNGH42008510, BNGH42008649, BNGH42008743, BNGH42008832, BNGH42008943, BNGH42009021, BRACE1000020, BRACE1000047, BRACE1000051, BRACE1000065, BRACE1000073, BRACE1000084, BRACE1000087, BRACE1000092, BRACE1000093, BRACE1000101, BRACE1000114, BRACE1000124, BRACE1000166, BRACE1000169, BRACE1000187, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000307, BRACE2000331, BRACE2000332, BRACE2000347, BRACE2000351, BRACE2000368, BRACE2000392, BRACE2000441,

BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000753, BRACE2000815, BRACE2000864, BRACE2000885, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001117, BRACE2001151, BRACE2001154, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374,

BRACE2001375, BRACE2001423, BRACE2001424, BRACE2001445, BRACE2001453, BRACE2001455, BRACE2001492, BRACE2001496, BRACE2001534, BRACE2001543, BRACE2001564, BRACE2001588, BRACE2001607, BRACE2001626, BRACE2001677, BRACE2001692, BRACE2001705, BRACE2001709, BRACE2001711, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001898, BRACE2001923, BRACE2001942, BRACE2001944, BRACE2001954, BRACE2001971, BRACE2001972, BRACE2001973, BRACE2002026, BRACE2002034, BRACE2002050, BRACE2002081, BRACE2002139, BRACE2002142, BRACE2002151, BRACE2002157, BRACE2002176, BRACE2002202, BRACE2002206,

BRACE2002227, BRACE2002260, BRACE2002297, BRACE2002304, BRACE2002356, BRACE2002392, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002613, BRACE2002614, BRACE2002617, BRACE2002623, BRACE2002635, BRACE2002649, BRACE2002676, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002807, BRACE2002812, BRACE2002834, BRACE2002849, BRACE2002857, BRACE2002884, BRACE2002896, BRACE2002948, BRACE2003037, BRACE2003078, BRACE2003097, BRACE2003110,

BRACE2003168, BRACE2003199, BRACE2003210, BRACE2003269, BRACE2003285, BRACE2003304, BRACE2003319, BRACE2003398, BRACE2003420, BRACE2003428, BRACE2003431, BRACE2003512, BRACE2003516, BRACE2003527, BRACE2003539, BRACE2003540, BRACE2003594, BRACE2003598, BRACE2003609, BRACE2003614, BRACE2003639, BRACE2003700, BRACE2003713, BRACE2003766, BRACE2003785, BRACE2003788, BRACE2003800, BRACE2003802, BRACE2003825, BRACE2003845, BRACE2003846, BRACE2003847, BRACE2003848, BRACE2003873, BRACE2003885, BRACE2003887, BRACE2003892, BRACE2003904, BRACE2003905, BRACE2003928, BRACE2003954, BRACE2003982, BRACE2003987, BRACE2005034, BRACE2005047, BRACE2005063, BRACE2005083, BRACE2005090, BRACE2005099, BRACE2005118,

BRACE2005124, BRACE2005138, BRACE2005151, BRACE2005160, BRACE2005169, BRACE2005189, BRACE2005193, BRACE2005196, BRACE2005198, BRACE2005216, BRACE2005231, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005433, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898,

BRACE2005904, BRACE2005906, BRACE2005907, BRACE2005911, BRACE2005949, BRACE2005981, BRACE2005991, BRACE2006055, BRACE2006072, BRACE2006075, BRACE2006084, BRACE2006089, BRACE2006102, BRACE2006105, BRACE2006122, BRACE2006137, BRACE2006143, BRACE2006167, BRACE2006174, BRACE2006240, BRACE2006245, BRACE2006249, BRACE2006274, BRACE2006281, BRACE2006298, BRACE2006344, BRACE2006354, BRACE2006363, BRACE2006378, BRACE2006380, BRACE2006393, BRACE2006413, BRACE2006417, BRACE2006453, BRACE2006459, BRACE2006488, BRACE2006534, BRACE2006540, BRACE2006564, BRACE2006587, BRACE2006598, BRACE2006636, BRACE2006685, BRACE2006703, BRACE2006743, BRACE2006783, BRACE2006821, BRACE2006833, BRACE2006859, BRACE2006871,

BRACE2006909, BRACE2006911, BRACE2006924, BRACE2006935, BRACE2006937, BRACE2006944, BRACE2006951, BRACE2006982, BRACE2006986, BRACE2007013, BRACE2007064, BRACE2007068, BRACE2007087, BRACE2007109, BRACE2007138, BRACE2007174, BRACE2007197, BRACE2007201, BRACE2007203, BRACE2007232, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007522, BRACE2007527, BRACE2007538, BRACE2007563, BRACE2007577, BRACE2007621, BRACE2007640, BRACE2007641, BRACE2007646, BRACE2007663, BRACE2007682, BRACE2007685, BRACE2007708, BRACE2007727, BRACE2007749, BRACE2007760, BRACE2007761,

BRACE2007764, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008097, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008361, BRACE2008380, BRACE2008385, BRACE2008399, BRACE2008401, BRACE2008402, BRACE2008410, BRACE2008420, BRACE2008443, BRACE2008473, BRACE2008480, BRACE2008481, BRACE2008488, BRACE2008495, BRACE2008500, BRACE2008553, BRACE2008594,

BRACE2008604, BRACE2008615, BRACE2008622, BRACE2008629, BRACE2008655, BRACE2008656, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008948, BRACE2008960, BRACE2008999, BRACE2009014, BRACE2009016, BRACE2009031, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009293, BRACE2009307, BRACE2009311, BRACE2009318, BRACE2009323, BRACE2009366, BRACE2009421, BRACE2009428, BRACE2009434, BRACE2009437,

BRACE2009483, BRACE2009533, BRACE2009558, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646,

BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317, BRACE2012510, BRACE2012528, BRACE2012625, BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132,

BRACE2015262, BRACE2015270, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016362, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017410, BRACE2017438, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847,

BRACE2019004, BRACE2019044, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021014, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021708, BRACE2021721, BRACE2021932, BRACE2021936, BRACE2022030, BRACE2022158,

BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294,

BRACE2026404, BRACE2026675, BRACE2026725, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148,

BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170,

BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300,

BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002298, BRACE3002344, BRACE3002390, BRACE3002420, BRACE3002508, BRACE3002541, BRACE3002756, BRACE3003004, BRACE3003053, BRACE3003192, BRACE3003413, BRACE3003595,

BRACE3003698, BRACE3003866, BRACE3003920, BRACE3004046, BRACE3004058, BRACE3004113, BRACE3004150, BRACE3004205, BRACE3004358, BRACE3004435, BRACE3004477, BRACE3004767, BRACE3004772, BRACE3004783, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005938, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642,

BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010702, BRACE3010862, BRACE3011271, BRACE3011421, BRACE3011447, BRACE3011505, BRACE3011634, BRACE3011774, BRACE3012357, BRACE3012574, BRACE3012806,

BRACE3012930, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194,

BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931,

BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760,

BRACE3039288, BRACE3039358, BRACE3039378, BRACE3039454, BRACE3040012, BRACE3040239, BRACE3040504, BRACE3040644, BRACE3040856, BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189,

BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2011880,

BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000388, BRAMY2000411, BRAMY2000508, BRAMY2000562, BRAMY2000585, BRAMY2000653, BRAMY2000814, BRAMY2000946, BRAMY2000981, BRAMY2000987, BRAMY2001068, BRAMY2001114, BRAMY2001203,

BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004387, BRAMY2004492,

BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333,

BRAMY2008382, BRAMY2008563, BRAMY2008583, BRAMY2008977, BRAMY2008979, BRAMY2009013, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811,

BRAMY2011841, BRAMY2011846, BRAMY2011849, BRAMY2011872, BRAMY2011961, BRAMY2012017, BRAMY2012119, BRAMY2012162, BRAMY2012163, BRAMY2012277, BRAMY2012340, BRAMY2012426, BRAMY2012497, BRAMY2012517, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2015211, BRAMY2015251, BRAMY2015311, BRAMY2015420, BRAMY2015503, BRAMY2015516, BRAMY2015550,

BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016020, BRAMY2016070, BRAMY2016251, BRAMY2016325, BRAMY2016327, BRAMY2016386, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019600, BRAMY2019643, BRAMY2019710,

BRAMY2019963, BRAMY2019985, BRAMY2019989, BRAMY2020050, BRAMY2020058, BRAMY2020106, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022796, BRAMY2022929, BRAMY2022980, BRAMY2022984, BRAMY2023060,

BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023729, BRAMY2023786, BRAMY2023852, BRAMY2023863, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395,

BRAMY2026405, BRAMY2026533, BRAMY2026538, BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027739, BRAMY2027752, BRAMY2027782, BRAMY2028072, BRAMY2028183, BRAMY2028188, BRAMY2028269, BRAMY2028282, BRAMY2028472, BRAMY2028516, BRAMY2028565, BRAMY2028593, BRAMY2028682, BRAMY2028783, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768,

BRAMY2030799, BRAMY2030859, BRAMY2030915, BRAMY2031317, BRAMY2031377, BRAMY2031442, BRAMY2031516, BRAMY2032014, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387,

BRAMY2036396, BRAMY2036567, BRAMY2036615, BRAMY2036699, BRAMY2036913, BRAMY2036918, BRAMY2036968, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040095, BRAMY2040159, BRAMY2040214, BRAMY2040478, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244,

BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401,

BRAMY3001409, BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3002991, BRAMY3003026, BRAMY3003109, BRAMY3003205, BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937,

BRAMY3009158, BRAMY3009207, BRAMY3009491, BRAMY3009556, BRAMY3009782, BRAMY3009811, BRAMY3009904, BRAMY3010264, BRAMY3010321, BRAMY3010411, BRAMY3010492, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000037, BRAWH1000045, BRAWH1000083, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082,

BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417, BRAWH2000443, BRAWH2000471, BRAWH2000476, BRAWH2000482, BRAWH2000494, BRAWH2000500, BRAWH2000522, BRAWH2000554, BRAWH2000595, BRAWH2000636, BRAWH2000643, BRAWH2000651, BRAWH2000682, BRAWH2000686, BRAWH2000752, BRAWH2000859, BRAWH2000866, BRAWH2000892, BRAWH2000901, BRAWH2000926, BRAWH2000944, BRAWH2000984, BRAWH2001019, BRAWH2001037, BRAWH2001052, BRAWH2001103, BRAWH2001129, BRAWH2001137, BRAWH2001159, BRAWH2001186, BRAWH2001203, BRAWH2001238, BRAWH2001239, BRAWH2001255,

BRAWH2001321, BRAWH2001355, BRAWH2001378, BRAWH2001395, BRAWH2001406, BRAWH2001412, BRAWH2001418, BRAWH2001424, BRAWH2001436, BRAWH2001438, BRAWH2001459, BRAWH2001461, BRAWH2001492, BRAWH2001503, BRAWH2001519, BRAWH2001530, BRAWH2001535, BRAWH2001589, BRAWH2001662, BRAWH2001666, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002601, BRAWH2002623, BRAWH2002630, BRAWH2002635, BRAWH2002662, BRAWH2002725, BRAWH2002761, BRAWH2002811, BRAWH2002861, BRAWH2002911, BRAWH2002963,

BRAWH2002967, BRAWH2002976, BRAWH2003000, BRAWH2003135, BRAWH2003240, BRAWH2003307, BRAWH2003355, BRAWH2003366, BRAWH2003662, BRAWH2003689, BRAWH2003693, BRAWH2003818, BRAWH2003832, BRAWH2003948, BRAWH2003964, BRAWH2004044, BRAWH2004068, BRAWH2004078, BRAWH2004136, BRAWH2004217, BRAWH2004580, BRAWH2004731, BRAWH2004779, BRAWH2004842, BRAWH2004884, BRAWH2004923, BRAWH2005008, BRAWH2005068, BRAWH2005074, BRAWH2005315, BRAWH2005517, BRAWH2005524, BRAWH2005533, BRAWH2005578, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960,

BRAWH2006989, BRAWH2006996, BRAWH2007069, BRAWH2007308, BRAWH2007406, BRAWH2007550, BRAWH2007570, BRAWH2007591, BRAWH2007605, BRAWH2007658, BRAWH2007664, BRAWH2007800, BRAWH2007808, BRAWH2007825, BRAWH2007862, BRAWH2007890, BRAWH2008058, BRAWH2008128, BRAWH2008255, BRAWH2008292, BRAWH2008297, BRAWH2008366, BRAWH2008380, BRAWH2008540, BRAWH2008577, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009304, BRAWH2009307, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084,

BRAWH2010318, BRAWH2010329, BRAWH2010354, BRAWH2010364, BRAWH2010536, BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012161, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013866, BRAWH2013871, BRAWH2013914, BRAWH2013941,

BRAWH2013955, BRAWH2014053, BRAWH2014234, BRAWH2014379, BRAWH2014414, BRAWH2014473, BRAWH2014511, BRAWH2014645, BRAWH2014662, BRAWH2014717, BRAWH2014729, BRAWH2014876, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433,

BRAWH2017523, BRAWH2017534, BRAWH2017542, BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018729, BRAWH2018745, BRAWH2018875, BRAWH2019053, BRAWH2019055, BRAWH2019198, BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002600, BRAWH3002819, BRAWH3003019, BRAWH3003136,

BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666, BRAWH3005037, BRAWH3005047, BRAWH3005132, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461,

BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928,

BRAWH3021012, BRAWH3021545, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772,

BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN2000148, BRCAN2000149, BRCAN2000209, BRCAN2000239,

BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401,

BRCAN2006411, BRCAN2006450, BRCAN2006955, BRCAN2007119, BRCAN2007144, BRCAN2007409, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2009432, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015402, BRCAN2015464, BRCAN2015719, BRCAN2015757, BRCAN2015886, BRCAN2016025,

BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2025009, BRCAN2025093, BRCAN2025349, BRCAN2025368, BRCAN2025712, BRCAN2025982, BRCAN2026117, BRCAN2026340, BRCAN2026711, BRCAN2027310,

BRCAN2027334, BRCAN2027355, BRCAN2027513, BRCAN2027593, BRCAN2027970, BRCAN2028021, BRCAN2028040, BRCAN2028319, BRCAN2028338, BRCAN2028378, BRCAN2028460, BRCAN2028545, BRCAN2028634, BRCAN2028702, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003513, BRCOC2003732, BRCOC2003740, BRCOC2004036, BRCOC2004354, BRCOC2004455, BRCOC2004950, BRCOC2005542,

BRCOC2005903, BRCOC2005951, BRCOC2006164, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012172, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944,

BRCOC2016525, BRCOC2016661, BRCOC2017061, BRCOC2017652, BRCOC2017856, BRCOC2018775, BRCOC2018877, BRCOC2019549, BRCOC2019841, BRCOC2019896, BRCOC2019903, BRCOC2019910, BRCOC2019934, BRCOC2020122, BRCOC2020142, BRHIP1000058, BRHIP1000072, BRHIP1000104, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000621, BRHIP2000691, BRHIP2000819, BRHIP2000826, BRHIP2000920, BRHIP2001074, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172,

BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007741, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414,

BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011491, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387,

BRHIP2014954, BRHIP2015153, BRHIP2015224, BRHIP2015356, BRHIP2015360, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018712, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020799, BRHIP2020842, BRHIP2020859,

BRHIP2020930, BRHIP2021102, BRHIP2021289, BRHIP2021495, BRHIP2021615, BRHIP2021744, BRHIP2021858, BRHIP2021929, BRHIP2022006, BRHIP2022054, BRHIP2022221, BRHIP2022228, BRHIP2022298, BRHIP2022467, BRHIP2022708, BRHIP2023071, BRHIP2023272, BRHIP2023292, BRHIP2023309, BRHIP2023369, BRHIP2023735, BRHIP2023773, BRHIP2023860, BRHIP2023869, BRHIP2024046, BRHIP2024165, BRHIP2024201, BRHIP2024347, BRHIP2024398, BRHIP2024549, BRHIP2024742, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017,

BRHIP2027054, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000017, BRHIP3000060, BRHIP3000070, BRHIP3000081, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395,

BRHIP3003484, BRHIP3003644, BRHIP3003688, BRHIP3003795, BRHIP3003984, BRHIP3004193, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007223, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405,

BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558,

BRHIP3017637, BRHIP3017855, BRHIP3018278, BRHIP3018784, BRHIP3019643, BRHIP3019824, BRHIP3019880, BRHIP3019956, BRHIP3020155, BRHIP3020182, BRHIP3020733, BRHIP3021019, BRHIP3021499, BRHIP3021534, BRHIP3021778, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244,

BRSSN2000293, BRSSN2000295, BRSSN2000312, BRSSN2000518, BRSSN2000561, BRSSN2000566, BRSSN2000684, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2002857, BRSSN2002859, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464,

BRSSN2008549, BRSSN2008797, BRSSN2009119, BRSSN2009378, BRSSN2009389, BRSSN2009395, BRSSN2009518, BRSSN2009627, BRSSN2009630, BRSSN2009702, BRSSN2010019, BRSSN2010110, BRSSN2010587, BRSSN2010596, BRSSN2010830, BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014636, BRSSN2014685, BRSSN2015199, BRSSN2015238, BRSSN2015369, BRSSN2015493, BRSSN2015497, BRSSN2015554, BRSSN2015707,

BRSSN2015773, BRSSN2015907, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367,

BRSTN2008418, BRSTN2008457, BRSTN2008475, BRSTN2008930, BRSTN2009247, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451, BRSTN2013171, BRSTN2013354, BRSTN2013931, BRSTN2014633, BRSTN2014751, BRSTN2014770, BRSTN2015015, BRSTN2015978, BRSTN2016066, BRSTN2016335, BRSTN2016678, BRSTN2016892, BRSTN2016918, BRSTN2016992, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063,

BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002741, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005942, BRTHA2005956, BRTHA2006075,

BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012466,

BRTHA2012555, BRTHA2012562, BRTHA2012714, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016577, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304,

BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845,

BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003704, BRTHA3004307, BRTHA3004432, BRTHA3004475, BRTHA3004502,

BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007532, BRTHA3007662, BRTHA3007685, BRTHA3007769, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010366, BRTHA3010469, BRTHA3010530, BRTHA3010540, BRTHA3010717, BRTHA3011149, BRTHA3011187, BRTHA3011194, BRTHA3011229, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547,

BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505,

BRTHA3028782, CD34C2000051, CD34C2000152, CD34C2000175, CD34C2000443, CD34C3000125, CD34C3000252, CD34C3000314, CD34C3000424, CD34C3000494, CERVX1000042, CERVX2000812, CERVX2000968, CERVX2002006, CERVX2002013, CERVX2002430, CHONS1000058, CHONS2000172, CHONS2000797, CHONS2001287, CHONS2001382, CHONS2001797, CHONS2001834, CHONS2002419, COLON1000030, COLON1000084, COLON1000135, COLON2000470, COLON2000557, COLON2000568, COLON2001721, COLON2001829, COLON2001866, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105,

COLON2009337, COLON2009499, CORDB1000140, CORDB2000061, CORDB2000127, CORDB2000541, CTONG1000010, CTONG1000020, CTONG1000022, CTONG1000040, CTONG1000056, CTONG1000062, CTONG1000086, CTONG1000087, CTONG1000088, CTONG1000093, CTONG1000097, CTONG1000113, CTONG1000137, CTONG1000165, CTONG1000180, CTONG1000181, CTONG1000241, CTONG1000283, CTONG1000288, CTONG1000302, CTONG1000341, CTONG1000476, CTONG1000488, CTONG1000508, CTONG1000540, CTONG2000031, CTONG2000034, CTONG2000201, CTONG2000218, CTONG2000279, CTONG2000303, CTONG2000330, CTONG2000395, CTONG2000465, CTONG2000480, CTONG2000508, CTONG2000766, CTONG2000771, CTONG2000819, CTONG2000827, CTONG2000846, CTONG2000855, CTONG2000977, CTONG2001139,

CTONG2001226, CTONG2001366, CTONG2001413, CTONG2001428, CTONG2001524, CTONG2001533, CTONG2001598, CTONG2001681, CTONG2001724, CTONG2001749, CTONG2001800, CTONG2001820, CTONG2001858, CTONG2001877, CTONG2001911, CTONG2001955, CTONG2002066, CTONG2002095, CTONG2002143, CTONG2002395, CTONG2002417, CTONG2002418, CTONG2002558, CTONG2002590, CTONG2002721, CTONG2002766, CTONG2002803, CTONG2002816, CTONG2002820, CTONG2002832, CTONG2002841, CTONG2002965, CTONG2002999, CTONG2003028, CTONG2003100, CTONG2003115, CTONG2003189, CTONG2003245, CTONG2003293, CTONG2003298, CTONG2003348, CTONG2003350, CTONG2003361, CTONG2003372, CTONG2003375, CTONG2003427, CTONG2003517, CTONG2003524, CTONG2003599, CTONG2003680,

CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004423, CTONG2004454, CTONG2004487, CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005278, CTONG2005290, CTONG2005374, CTONG2005399, CTONG2005468, CTONG2005567, CTONG2005585, CTONG2005615, CTONG2005635, CTONG2005775, CTONG2005795, CTONG2005890, CTONG2005904, CTONG2006004, CTONG2006129, CTONG2006223, CTONG2006235, CTONG2006273, CTONG2006310, CTONG2006377, CTONG2006393, CTONG2006412, CTONG2006449,

CTONG2006515, CTONG2006524, CTONG2006568, CTONG2006611, CTONG2006620, CTONG2006666, CTONG2006691, CTONG2006798, CTONG2006836, CTONG2006942, CTONG2006964, CTONG2007007, CTONG2007009, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007586, CTONG2007623, CTONG2007681, CTONG2007779, CTONG2007898, CTONG2007959, CTONG2007970, CTONG2008014, CTONG2008184, CTONG2008262, CTONG2008269, CTONG2008321, CTONG2008343, CTONG2008402, CTONG2008405, CTONG2008466, CTONG2008521, CTONG2008562, CTONG2008577, CTONG2008595, CTONG2008621, CTONG2008689, CTONG2008720, CTONG2008721,

CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2009963, CTONG2010018, CTONG2010024, CTONG2010116, CTONG2010133, CTONG2010148, CTONG2010276, CTONG2010330, CTONG2010408, CTONG2010508, CTONG2010566, CTONG2010623, CTONG2010649, CTONG2010652,

CTONG2010803, CTONG2011188, CTONG2011429, CTONG2011474, CTONG2011676, CTONG2011716, CTONG2011765, CTONG2011779, CTONG2011801, CTONG2011815, CTONG2011822, CTONG2011920, CTONG2011985, CTONG2012029, CTONG2012050, CTONG2012077, CTONG2012101, CTONG2012123, CTONG2012158, CTONG2012230, CTONG2012422, CTONG2012425, CTONG2012447, CTONG2012453, CTONG2012473, CTONG2012476, CTONG2012533, CTONG2012554, CTONG2012564, CTONG2012661, CTONG2012724, CTONG2012738, CTONG2012745, CTONG2012758, CTONG2012843, CTONG2012901, CTONG2012996, CTONG2013128, CTONG2013178, CTONG2013284, CTONG2013339, CTONG2013348, CTONG2013352, CTONG2013381, CTONG2013393, CTONG2013511, CTONG2013630, CTONG2013803, CTONG2013907, CTONG2013934,

CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014369, CTONG2014389, CTONG2014491, CTONG2014630, CTONG2014635, CTONG2014646, CTONG2014705, CTONG2014898, CTONG2014946, CTONG2014954, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016185, CTONG2016217, CTONG2016355, CTONG2016408, CTONG2016499, CTONG2016505, CTONG2016559, CTONG2016658, CTONG2016775,

CTONG2016797, CTONG2016824, CTONG2016846, CTONG2016869, CTONG2016904, CTONG2016931, CTONG2016942, CTONG2016953, CTONG2017021, CTONG2017094, CTONG2017292, CTONG2017409, CTONG2017429, CTONG2017444, CTONG2017458, CTONG2017500, CTONG2017604, CTONG2017650, CTONG2017798, CTONG2017804, CTONG2017814, CTONG2017910, CTONG2017939, CTONG2017989, CTONG2017998, CTONG2018062, CTONG2018069, CTONG2018147, CTONG2018211, CTONG2018217, CTONG2018279, CTONG2018334, CTONG2018343, CTONG2018379, CTONG2018413, CTONG2018483, CTONG2018629, CTONG2018637, CTONG2018652, CTONG2018653, CTONG2018655, CTONG2018735, CTONG2018739, CTONG2018808, CTONG2018843, CTONG2018898, CTONG2018900, CTONG2018976, CTONG2019063, CTONG2019144,

CTONG2019232, CTONG2019400, CTONG2019652, CTONG2019704, CTONG2019788, CTONG2019822, CTONG2019833, CTONG2020031, CTONG2020108, CTONG2020127, CTONG2020171, CTONG2020374, CTONG2020378, CTONG2020411, CTONG2020522, CTONG2020638, CTONG2020806, CTONG2020831, CTONG2020974, CTONG2021024, CTONG2021132, CTONG2021168, CTONG2021289, CTONG2022153, CTONG2022601, CTONG2022835, CTONG2023021, CTONG2023449, CTONG2023512, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959,

CTONG2028097, CTONG2028486, CTONG2028518, CTONG2028687, CTONG2028758, CTONG3000686, CTONG3000707, CTONG3000896, CTONG3001123, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002728, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317, CTONG3004397, CTONG3004550, CTONG3004576, CTONG3004607, CTONG3004712, CTONG3005325, CTONG3005648, CTONG3005713, CTONG3005803, CTONG3005813, CTONG3006067, CTONG3006073,

CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009028, CTONG3009227, CTONG3009287, CTONG3009328, CTONG3009385, D3OST1000066, D3OST1000238, D3OST1000267, D3OST2000184, D3OST2000618, D3OST2000654, D3OST2000734, D3OST2001328, D3OST2001669, D3OST2001670, D3OST2001788, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000195, D3OST3000258, D3OST3000291, D3OST3000388,

D3OST3000493, D6OST2000127, D6OST2000169, D6OST2000245, D6OST2000358, D6OST2000464, D6OST2000507, D9OST2000031, D9OST2000278, D9OST2000440, D9OST2000869, D9OST2001319, D9OST2001433, D9OST2002397, D9OST2002608, D9OST2002673, D9OST2003106, D9OST2003108, D9OST2003137, D9OST2003397, D9OST2003580, D9OST2003594, D9OST2003762, D9OST2003791, D9OST2003989, D9OST2004018, D9OST2004073, D9OST2004417, DFNES1000003, DFNES1000107, DFNES2000143, DFNES2000146, DFNES2000153, DFNES2000212, DFNES2000335, DFNES2000426, DFNES2000443, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2001829,

DFNES2002220, DFNES2002509, DFNES2002550, DFNES2002588, DFNES2002817, DFNES2002966, DFNES2003081, DFNES2003255, DFNES2003742, DFNES2004346, DFNES2004354, DFNES2004383, DFNES2004608, DFNES2004684, DFNES2005266, DFNES2005527, DFNES2005690, DFNES2005766, DFNES2005779, DFNES2005875, DFNES2006346, DFNES2006944, DFNES2007113, DFNES2007299, DFNES2007332, DFNES2007641, DFNES2007757, DFNES2008088, DFNES2008160, DFNES2008280, DFNES2008881, DFNES2009482, DFNES2010502, DFNES2011192, DFNES2011221, DFNES2011245, DFNES2011380, ERLTF1000021, ERLTF2000324, ERLTF2000465, FCBBF1000023, FCBBF1000038, FCBBF1000063, FCBBF1000069, FCBBF1000115, FCBBF1000117, FCBBF1000118, FCBBF1000121, FCBBF1000125, FCBBF1000131,

FCBBF1000155, FCBBF1000157, FCBBF1000171, FCBBF1000174, FCBBF1000198, FCBBF1000206, FCBBF1000220, FCBBF1000232, FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000403, FCBBF1000437, FCBBF1000476, FCBBF1000506, FCBBF1000509, FCBBF1000546, FCBBF1000550, FCBBF1000556, FCBBF1000574, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF1000760, FCBBF2000038, FCBBF2000087, FCBBF2000094,

FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473, FCBBF2000576, FCBBF2000677, FCBBF2000678, FCBBF2000733, FCBBF2000782, FCBBF2000808, FCBBF2000815, FCBBF2000825, FCBBF2000940, FCBBF2000951, FCBBF2001001, FCBBF2001088, FCBBF2001116, FCBBF2001183, FCBBF2001188, FCBBF2001211, FCBBF2001238, FCBBF2001243, FCBBF2001291, FCBBF2001299, FCBBF2001400, FCBBF2001427, FCBBF2001480, FCBBF2001529, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002370, FCBBF2002541, FCBBF2002626, FCBBF2002801,

FCBBF2002897, FCBBF2002898, FCBBF2002904, FCBBF2002928, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005645, FCBBF2005658, FCBBF2005733, FCBBF2005909, FCBBF2005966, FCBBF2006131, FCBBF2006380, FCBBF2006418, FCBBF2006452, FCBBF2006781, FCBBF2007012, FCBBF2007186, FCBBF2007265, FCBBF2007302, FCBBF2007556, FCBBF2007610, FCBBF2007633,

FCBBF2007840, FCBBF3000102, FCBBF3000110, FCBBF3000115, FCBBF3000120, FCBBF3000168, FCBBF3000175, FCBBF3000227, FCBBF3000229, FCBBF3000233, FCBBF3000269, FCBBF3000361, FCBBF3000367, FCBBF3000388, FCBBF3000462, FCBBF3000473, FCBBF3000501, FCBBF3000518, FCBBF3000536, FCBBF3000550, FCBBF3000847, FCBBF3001018, FCBBF3001020, FCBBF3001235, FCBBF3001281, FCBBF3001302, FCBBF3001377, FCBBF3001470, FCBBF3001594, FCBBF3001632, FCBBF3001818, FCBBF3001855, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305,

FCBBF3003373, FCBBF3003435, FCBBF3003475, FCBBF3003481, FCBBF3003557, FCBBF3003787, FCBBF3003800, FCBBF3003902, FCBBF3004041, FCBBF3004323, FCBBF3004390, FCBBF3004409, FCBBF3004502, FCBBF3004715, FCBBF3004842, FCBBF3004847, FCBBF3004955, FCBBF3005160, FCBBF3005218, FCBBF3005444, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008153, FCBBF3008159, FCBBF3008251, FCBBF3008311,

FCBBF3008362, FCBBF3008382, FCBBF3008475, FCBBF3008556, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288,

FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014355, FCBBF3014567, FCBBF3015100, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016928, FCBBF3016987, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729,

FCBBF3017873, FCBBF3017918, FCBBF3017974, FCBBF3018067, FCBBF3018173, FCBBF3018453, FCBBF3018591, FCBBF3018753, FCBBF3018796, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019714, FCBBF3019716, FCBBF3019784, FCBBF3019839, FCBBF3019867, FCBBF3019961, FCBBF3020030, FCBBF3020138, FCBBF3020140, FCBBF3020163, FCBBF3020232, FCBBF3020599, FCBBF3021026, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078,

FCBBF3023368, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028472, FCBBF3028528,

FCBBF3028582, FCBBF3028593, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000500, FCBBF4000529, FCBBF4000548, FCBBF5000041, FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000022, FEBRA1000030, FEBRA1000057, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000129, FEBRA2000210, FEBRA2000220, FEBRA2000228, FEBRA2000253,

FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000409, FEBRA2000415, FEBRA2000436, FEBRA2000438, FEBRA2000452, FEBRA2000454, FEBRA2000462, FEBRA2000468, FEBRA2000491, FEBRA2000504, FEBRA2000532, FEBRA2000536, FEBRA2000538, FEBRA2000575, FEBRA2000656, FEBRA2000659, FEBRA2000680, FEBRA2000690, FEBRA2000727, FEBRA2000734, FEBRA2000740, FEBRA2000757, FEBRA2000762, FEBRA2000771, FEBRA2000772, FEBRA2000782, FEBRA2000790, FEBRA2000793, FEBRA2000803, FEBRA2000805, FEBRA2000809, FEBRA2000815, FEBRA2000856, FEBRA2000862, FEBRA2000877, FEBRA2000880, FEBRA2000901, FEBRA2000909,

FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001245, FEBRA2001267, FEBRA2001294, FEBRA2001334, FEBRA2001351, FEBRA2001383, FEBRA2001438, FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001752, FEBRA2001772, FEBRA2001790, FEBRA2001814, FEBRA2001828, FEBRA2001867,

FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002429, FEBRA2002442, FEBRA2002456, FEBRA2002508, FEBRA2002525, FEBRA2002527, FEBRA2002552, FEBRA2002574, FEBRA2002601, FEBRA2002611, FEBRA2002628, FEBRA2002662, FEBRA2002682, FEBRA2002707, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308,

FEBRA2003327, FEBRA2003429, FEBRA2003436, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003822, FEBRA2003897, FEBRA2003907, FEBRA2004023, FEBRA2004026, FEBRA2004029, FEBRA2004053, FEBRA2004056, FEBRA2004091, FEBRA2004110, FEBRA2004191, FEBRA2004202, FEBRA2004219, FEBRA2004224, FEBRA2004237, FEBRA2004268, FEBRA2004329, FEBRA2004331, FEBRA2004443, FEBRA2004485, FEBRA2004490, FEBRA2004538, FEBRA2004592, FEBRA2004620, FEBRA2004651, FEBRA2004767, FEBRA2004813, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114,

FEBRA2005216, FEBRA2005291, FEBRA2005361, FEBRA2005377, FEBRA2005380, FEBRA2005427, FEBRA2005458, FEBRA2005476, FEBRA2005701, FEBRA2005726, FEBRA2005752, FEBRA2005778, FEBRA2005787, FEBRA2005788, FEBRA2005995, FEBRA2005998, FEBRA2006061, FEBRA2006092, FEBRA2006150, FEBRA2006165, FEBRA2006255, FEBRA2006315, FEBRA2006346, FEBRA2006354, FEBRA2006357, FEBRA2006368, FEBRA2006370, FEBRA2006379, FEBRA2006396, FEBRA2006401, FEBRA2006409, FEBRA2006418, FEBRA2006427, FEBRA2006476, FEBRA2006485, FEBRA2006491, FEBRA2006521, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017,

FEBRA2007176, FEBRA2007200, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007620, FEBRA2007622, FEBRA2007708, FEBRA2007714, FEBRA2007793, FEBRA2007801, FEBRA2007818, FEBRA2007823, FEBRA2007880, FEBRA2007900, FEBRA2007901, FEBRA2007931, FEBRA2007957, FEBRA2008009, FEBRA2008036, FEBRA2008086, FEBRA2008134, FEBRA2008135, FEBRA2008159, FEBRA2008165, FEBRA2008172, FEBRA2008201, FEBRA2008210, FEBRA2008265, FEBRA2008266, FEBRA2008282, FEBRA2008287, FEBRA2008311, FEBRA2008341, FEBRA2008360, FEBRA2008459, FEBRA2008468, FEBRA2008475, FEBRA2008481, FEBRA2008542, FEBRA2008583,

FEBRA2008660, FEBRA2008662, FEBRA2008681, FEBRA2008692, FEBRA2008741, FEBRA2008755, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117, FEBRA2009120, FEBRA2009162, FEBRA2009185, FEBRA2009240, FEBRA2009265, FEBRA2009289, FEBRA2009327, FEBRA2009328, FEBRA2009352, FEBRA2009362, FEBRA2009385, FEBRA2009416, FEBRA2009437, FEBRA2009478, FEBRA2009507, FEBRA2009514, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063, FEBRA2011090,

FEBRA2011356, FEBRA2011392, FEBRA2011414, FEBRA2011593, FEBRA2011665, FEBRA2012090, FEBRA2012120, FEBRA2012181, FEBRA2012195, FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502, FEBRA2017533, FEBRA2017680,

FEBRA2017736, FEBRA2017780, FEBRA2017811, FEBRA2017850, FEBRA2018051, FEBRA2018203, FEBRA2018407, FEBRA2018433, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019711, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022148, FEBRA2022204, FEBRA2022255, FEBRA2022322, FEBRA2022601, FEBRA2022696, FEBRA2022729,

FEBRA2022787, FEBRA2022911, FEBRA2022956, FEBRA2022963, FEBRA2022972, FEBRA2023227, FEBRA2023285, FEBRA2023351, FEBRA2023377, FEBRA2023399, FEBRA2023498, FEBRA2023550, FEBRA2023721, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025427, FEBRA2025463, FEBRA2025477, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEBRA2028516, FEHRT2000325,

FEHRT2000364, FEKID1000018, FELIV1000101, FELIV1000137, FELIV1000164, FELNG2000241, HCASM1000021, HCASM1000050, HCASM1000061, HCASM1000115, HCASM1000130, HCASM2000016, HCASM2000138, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405,

HCASM2009424, HCASM2009463, HCASM2009588, HCHON1000015, HCHON1000131, HCHON1000142, HCHON1000176, HCHON1000191, HCHON2000038, HCHON2000100, HCHON2000156, HCHON2000199, HCHON2000212, HCHON2000271, HCHON2000315, HCHON2000323, HCHON2000344, HCHON2000364, HCHON2000395, HCHON2000417, HCHON2000418, HCHON2000473, HCHON2000503, HCHON2000626, HCHON2000660, HCHON2000663, HCHON2000673, HCHON2000676, HCHON2000698, HCHON2000699, HCHON2000705, HCHON2000738, HCHON2000743, HCHON2000751, HCHON2000815, HCHON2000817, HCHON2000818, HCHON2000819, HCHON2000832, HCHON2000851, HCHON2000898, HCHON2000935, HCHON2000942, HCHON2000956, HCHON2000994, HCHON2001050, HCHON2001108, HCHON2001110, HCHON2001116, HCHON2001161,

HCHON2001214, HCHON2001233, HCHON2001269, HCHON2001450, HCHON2001453, HCHON2001497, HCHON2001519, HCHON2001523, HCHON2001535, HCHON2001546, HCHON2001548, HCHON2001577, HCHON2001598, HCHON2001604, HCHON2001607, HCHON2001646, HCHON2001665, HCHON2001712, HCHON2001768, HCHON2001801, HCHON2001853, HCHON2002123, HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005987,

HCHON2006250, HCHON2006459, HCHON2006714, HCHON2006722, HCHON2006770, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HCHON2010543, HEART1000010, HEART1000099, HEART1000102, HEART1000118, HEART1000132, HEART1000151, HEART1000173, HEART2000024, HEART2000035, HEART2000099, HEART2000298, HEART2000306, HEART2000309, HEART2000411, HEART2000418, HEART2000444, HEART2000448, HEART2000492, HEART2000568, HEART2000611, HEART2000959,

HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006195, HEART2006310, HEART2006334, HEART2006487, HEART2006557, HEART2006787, HEART2006789, HEART2006909, HEART2007031, HEART2007231, HEART2007443, HEART2007767, HEART2008108, HEART2008111, HEART2008257, HEART2008509, HEART2008994, HEART2009000, HEART2009599, HEART2009680, HEART2010391, HEART2010492, HELAC2000158, HELAC2000301,

HHDPC1000001, HHDPC1000065, HHDPC1000114, HHDPC1000118, HHDPC2000070, HHDPC2000102, HHDPC2000104, HHDPC2000258, HHDPC2000315, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006862, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030, HLUNG1000034, HLUNG1000037, HLUNG1000053,

HLUNG1000064, HLUNG1000084, HLUNG1000091, HLUNG1000099, HLUNG1000104, HLUNG1000105, HLUNG1000110, HLUNG1000151, HLUNG1000169, HLUNG2000004, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000340, HLUNG2000362, HLUNG2000363, HLUNG2000431, HLUNG2000480, HLUNG2000489, HLUNG2000549, HLUNG2000557, HLUNG2000570, HLUNG2000751, HLUNG2000761, HLUNG2000777, HLUNG2000846, HLUNG2000870, HLUNG2000926, HLUNG2000950, HLUNG2000955, HLUNG2000970, HLUNG2001013, HLUNG2001126, HLUNG2001144, HLUNG2001146, HLUNG2001214, HLUNG2001439, HLUNG2001507,

HLUNG2001677, HLUNG2001814, HLUNG2001996, HLUNG2002006, HLUNG2002050, HLUNG2002085, HLUNG2002145, HLUNG2002295, HLUNG2002303, HLUNG2002334, HLUNG2002405, HLUNG2002429, HLUNG2002465, HLUNG2002562, HLUNG2002659, HLUNG2002707, HLUNG2002811, HLUNG2002916, HLUNG2002942, HLUNG2002949, HLUNG2002958, HLUNG2002982, HLUNG2003003, HLUNG2003049, HLUNG2003061, HLUNG2003162, HLUNG2003246, HLUNG2003331, HLUNG2003335, HLUNG2003378, HLUNG2003497, HLUNG2003691, HLUNG2003710, HLUNG2003714, HLUNG2003716, HLUNG2003833, HLUNG2003872, HLUNG2003918, HLUNG2003991, HLUNG2004014, HLUNG2004037, HLUNG2004067, HLUNG2004154, HLUNG2004217, HLUNG2004273, HLUNG2004388, HLUNG2004416, HLUNG2004521, HLUNG2004534, HLUNG2004684,

HLUNG2004707, HLUNG2004774, HLUNG2004775, HLUNG2005012, HLUNG2005030, HLUNG2005045, HLUNG2005076, HLUNG2005133, HLUNG2005203, HLUNG2005479, HLUNG2005524, HLUNG2005656, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006812, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008139, HLUNG2008153, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521,

HLUNG2008833, HLUNG2008875, HLUNG2008885, HLUNG2008958, HLUNG2008959, HLUNG2009067, HLUNG2009225, HLUNG2009253, HLUNG2009303, HLUNG2009413, HLUNG2009729, HLUNG2010181, HLUNG2010464, HLUNG2010562, HLUNG2010845, HLUNG2011041, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282,

HLUNG2018626, HLUNG2019058, HSYRA1000131, HSYRA1000148, HSYRA2000044, HSYRA2000135, HSYRA2000190, HSYRA2000221, HSYRA2000237, HSYRA2000253, HSYRA2000255, HSYRA2000332, HSYRA2000371, HSYRA2000424, HSYRA2000507, HSYRA2000510, HSYRA2000629, HSYRA2000706, HSYRA2000760, HSYRA2000792, HSYRA2000832, HSYRA2000925, HSYRA2000927, HSYRA2001003, HSYRA2001103, HSYRA2001142, HSYRA2001153, HSYRA2001232, HSYRA2001255, HSYRA2001332, HSYRA2001353, HSYRA2001420, HSYRA2001484, HSYRA2001552, HSYRA2001567, HSYRA2001580, HSYRA2001595, HSYRA2001631, HSYRA2001983, HSYRA2002103, HSYRA2002590, HSYRA2003168, HSYRA2004550, HSYRA2004858, HSYRA2005456, HSYRA2005496, HSYRA2005628, HSYRA2005970, HSYRA2006049, HSYRA2006873,

HSYRA2007241, HSYRA2007338, HSYRA2007667, HSYRA2008154, HSYRA2008714, HSYRA2009102, IMR321000155, IMR321000158, IMR321000161, IMR321000165, IMR321000193, IMR321000207, IMR321000230, IMR321000242, IMR321000253, IMR322000010, IMR322000072, IMR322000107, IMR322000152, IMR322000164, IMR322000302, IMR322000374, IMR322000379, IMR322000383, IMR322000418, IMR322000518, IMR322000555, IMR322000604, IMR322000609, IMR322000656, IMR322000672, IMR322000742, IMR322000791, IMR322000819, IMR322000838, IMR322000859, IMR322000863, IMR322000917, IMR322000919, IMR322000953, IMR322000973, IMR322000984, IMR322001018, IMR322001167, IMR322001185, IMR322001245, IMR322001317, IMR322001318, IMR322001322, IMR322001332,

IMR322001380, IMR322001438, IMR322001452, IMR322001491, IMR322001534, IMR322001665, IMR322001670, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, JCMLC2000273, KIDNE1000008, KIDNE1000012, KIDNE1000015, KIDNE1000028, KIDNE1000036,

KIDNE1000064, KIDNE1000104, KIDNE1000121, KIDNE1000143, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000266, KIDNE2000287, KIDNE2000315, KIDNE2000317, KIDNE2000328, KIDNE2000330, KIDNE2000335, KIDNE2000349, KIDNE2000375, KIDNE2000394, KIDNE2000422, KIDNE2000493, KIDNE2000510, KIDNE2000513, KIDNE2000519, KIDNE2000555, KIDNE2000624, KIDNE2000665, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2000947, KIDNE2001117, KIDNE2001140, KIDNE2001269, KIDNE2001361, KIDNE2001373,

KIDNE2001713, KIDNE2001802, KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002252, KIDNE2002262, KIDNE2002265, KIDNE2002438, KIDNE2002483, KIDNE2002725, KIDNE2002768, KIDNE2002795, KIDNE2002798, KIDNE2002839, KIDNE2002845, KIDNE2002872, KIDNE2002882, KIDNE2002991, KIDNE2003185, KIDNE2003188, KIDNE2003233, KIDNE2003316, KIDNE2003335, KIDNE2003357, KIDNE2003373, KIDNE2003377, KIDNE2003507, KIDNE2003752, KIDNE2003764, KIDNE2003837, KIDNE2003941, KIDNE2003994, KIDNE2004034, KIDNE2004054, KIDNE2004084, KIDNE2004095, KIDNE2004115, KIDNE2004233, KIDNE2004262, KIDNE2004279,

KIDNE2004294, KIDNE2004295, KIDNE2004305, KIDNE2004344, KIDNE2004394, KIDNE2004519, KIDNE2004520, KIDNE2004531, KIDNE2004534, KIDNE2004562, KIDNE2004579, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006014, KIDNE2006030, KIDNE2006039, KIDNE2006053, KIDNE2006062, KIDNE2006092, KIDNE2006145, KIDNE2006149, KIDNE2006210, KIDNE2006299, KIDNE2006334,

KIDNE2006353, KIDNE2006376, KIDNE2006378, KIDNE2006465, KIDNE2006580, KIDNE2006652, KIDNE2006687, KIDNE2006733, KIDNE2006760, KIDNE2006775, KIDNE2006811, KIDNE2006820, KIDNE2006880, KIDNE2007005, KIDNE2007040, KIDNE2007077, KIDNE2007186, KIDNE2007222, KIDNE2007422, KIDNE2007569, KIDNE2007810, KIDNE2007811, KIDNE2007910, KIDNE2007944, KIDNE2008022, KIDNE2008069, KIDNE2008072, KIDNE2008117, KIDNE2008218, KIDNE2008315, KIDNE2008362, KIDNE2008378, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109,

KIDNE2009180, KIDNE2009191, KIDNE2009332, KIDNE2009367, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012453, KIDNE2012563, KIDNE2012601, KIDNE2012613, KIDNE2012710,

KIDNE2012745, KIDNE2012775, KIDNE2012784, KIDNE2012945, KIDNE2013045, KIDNE2013095, KIDNE2013158, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015483, KIDNE2015515, KIDNE2015546, KIDNE2015556, KIDNE2015598, KIDNE2015660, KIDNE2015710, KIDNE2015987, KIDNE2016000,

KIDNE2016036, KIDNE2016096, KIDNE2016101, KIDNE2016142, KIDNE2016188, KIDNE2016327, KIDNE2016388, KIDNE2016464, KIDNE2016539, KIDNE2016570, KIDNE2016918, KIDNE2017007, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018352, KIDNE2018462, KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000082, LIVER1000099,

LIVER1000104, LIVER1000111, LIVER1000126, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000033, LIVER2000037, LIVER2000158, LIVER2000216, LIVER2000237, LIVER2000247, LIVER2000360, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001835, LIVER2002638, LIVER2002644, LIVER2002660, LIVER2002702, LIVER2003008, LIVER2003065, LIVER2003234, LIVER2003511,

LIVER2003524, LIVER2003568, LIVER2003581, LIVER2003800, LIVER2003854, LIVER2004074, LIVER2004392, LIVER2004514, LIVER2004565, LIVER2005198, LIVER2005218, LIVER2005376, LIVER2005385, LIVER2005512, LIVER2005520, LIVER2005527, LIVER2005544, LIVER2005625, LIVER2005728, LIVER2005973, LIVER2006006, LIVER2006251, LIVER2006410, LIVER2006469, LIVER2006644, LIVER2006764, LIVER2007415, LIVER2007548, LIVER2007568, LIVER2007721, LIVER2008053, LIVER2008465, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000100, MAMGL1000132, MAMGL1000151, MAMGL1000178,

MAMGL1000182, MAMGL1000184, MESAN1000032, MESAN1000035, MESAN1000050, MESAN1000079, MESAN1000080, MESAN1000121, MESAN1000147, MESAN1000180, MESAN2000023, MESAN2000026, MESAN2000067, MESAN2000092, MESAN2000149, MESAN2000167, MESAN2000291, MESAN2000337, MESAN2000434, MESAN2000462, MESAN2000501, MESAN2000711, MESAN2000815, MESAN2000894, MESAN2000909, MESAN2001627, MESAN2001770, MESAN2001979, MESAN2001980, MESAN2002113, MESAN2002122, MESAN2002147, MESAN2002186, MESAN2002424, MESAN2002519, MESAN2002687, MESAN2002709, MESAN2002844, MESAN2002978, MESAN2003037, MESAN2003039, MESAN2003101, MESAN2003190, MESAN2003322, MESAN2003490, MESAN2003529, MESAN2003646, MESAN2003662, MESAN2003709, MESAN2003851,

MESAN2004138, MESAN2004288, MESAN2004575, MESAN2005284, MESAN2005371, MESAN2005633, MESAN2005689, MESAN2005724, MESAN2005808, MESAN2005811, MESAN2006022, MESAN2006043, MESAN2006328, MESAN2006401, MESAN2006580, MESAN2006599, MESAN2006953, MESAN2006971, MESAN2007032, MESAN2007440, MESAN2007597, MESAN2007755, MESAN2008222, MESAN2008334, MESAN2008460, MESAN2008536, MESAN2008679, MESAN2008729, MESAN2008821, MESAN2008926, MESAN2009019, MESAN2009081, MESAN2009105, MESAN2009311, MESAN2009418, MESAN2009503, MESAN2009522, MESAN2010031, MESAN2010114, MESAN2010312, MESAN2010321, MESAN2010664, MESAN2010681, MESAN2011545, MESAN2011597, MESAN2011632, MESAN2011977, MESAN2012586, MESAN2012708, MESAN2012735,

MESAN2013019, MESAN2013022, MESAN2013183, MESAN2013211, MESAN2013283, MESAN2013284, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016552, MESAN2016965, MESAN2017133, MESAN2017152, MESAN2017373, MESAN2017417, MESAN2018209, MESAN2018576, MESAN2018670, MESTC1000042, MESTC1000147, MESTC2000150, MESTC2000153, MESTC2000170, N1ESE2000698, NB9N41000047, NB9N41000121, NB9N41000135, NB9N41000142, NB9N41000146, NB9N41000340, NB9N42000104, NB9N42000122, NB9N42000196,

NB9N42000281, NB9N42000314, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062, NHNPC2000212, NHNPC2000606, NHNPC2000877, NHNPC2001108, NHNPC2001223, NHNPC2001312, NHNPC2001816, NHNPC2002565, NHNPC2002749, NOVAR1000015, NOVAR1000102, NOVAR2000136, NOVAR2000352, NOVAR2000412, NOVAR2000710,

NOVAR2000783, NOVAR2000962, NOVAR2001108, NOVAR2001620, NOVAR2001783, NOVAR2001876, NOVAR2002039, NT2NE1000004, NT2NE1000014, NT2NE1000018, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000222, NT2NE2000284, NT2NE2000299, NT2NE2000353, NT2NE2000356, NT2NE2000369, NT2NE2000374, NT2NE2000386, NT2NE2000390, NT2NE2000392, NT2NE2000455, NT2NE2000470, NT2NE2000488, NT2NE2000517, NT2NE2000536, NT2NE2000546, NT2NE2000548, NT2NE2000550, NT2NE2000586, NT2NE2000612, NT2NE2000636, NT2NE2000658,

NT2NE2000706, NT2NE2000763, NT2NE2000802, NT2NE2000808, NT2NE2000809, NT2NE2000864, NT2NE2000909, NT2NE2000913, NT2NE2000950, NT2NE2000951, NT2NE2000980, NT2NE2001005, NT2NE2001021, NT2NE2001040, NT2NE2001048, NT2NE2001049, NT2NE2001142, NT2NE2001156, NT2NE2001176, NT2NE2001180, NT2NE2001181, NT2NE2001188, NT2NE2001191, NT2NE2001247, NT2NE2001252, NT2NE2001324, NT2NE2001326, NT2NE2001364, NT2NE2001372, NT2NE2001397, NT2NE2001403, NT2NE2001428, NT2NE2001432, NT2NE2001442, NT2NE2001524, NT2NE2001598, NT2NE2001617, NT2NE2001623, NT2NE2001626, NT2NE2001634, NT2NE2001648, NT2NE2001660, NT2NE2001666, NT2NE2001677, NT2NE2001697, NT2NE2001698, NT2NE2001723, NT2NE2001725, NT2NE2001874, NT2NE2001889,

NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2003150, NT2NE2003185, NT2NE2003252, NT2NE2003280, NT2NE2003308, NT2NE2003309, NT2NE2003315, NT2NE2003393, NT2NE2003408, NT2NE2003457, NT2NE2003485, NT2NE2003540, NT2NE2003569, NT2NE2003571, NT2NE2003705, NT2NE2003752, NT2NE2003887, NT2NE2003921, NT2NE2004255, NT2NE2004378, NT2NE2004490, NT2NE2004519, NT2NE2004606, NT2NE2004740, NT2NE2004787, NT2NE2004916, NT2NE2005035, NT2NE2005223, NT2NE2005276, NT2NE2005317, NT2NE2005323, NT2NE2005358, NT2NE2005395, NT2NE2005419, NT2NE2005517, NT2NE2005537, NT2NE2005555, NT2NE2005588,

NT2NE2005676, NT2NE2005720, NT2NE2005784, NT2NE2005804, NT2NE2005821, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295,

NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010854, NT2NE2011033, NT2NE2011036, NT2NE2011107, NT2NE2011154, NT2NE2011221, NT2NE2011411, NT2NE2011485, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012790, NT2NE2012847, NT2NE2013019, NT2NE2013081, NT2NE2013189, NT2NE2013217, NT2NE2013547, NT2NE2014013, NT2NE2014028, NT2NE2014113,

NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490,

NT2NE2018594, NT2NE2018739, NT2NE2018916, NT2NE2019056, NT2NE2019076, NT2NE2019091, NT2NE2019099, NT2RI1000016, NT2RI1000035, NT2RI1000048, NT2RI1000127, NT2RI1000164, NT2RI1000172, NT2RI2000004, NT2RI2000007, NT2RI2000116, NT2RI2000117, NT2RI2000128, NT2RI2000133, NT2RI2000167, NT2RI2000173, NT2RI2000187, NT2RI2000263, NT2RI2000270, NT2RI2000276, NT2RI2000284, NT2RI2000294, NT2RI2000313, NT2RI2000341, NT2RI2000344, NT2RI2000348, NT2RI2000412, NT2RI2000421, NT2RI2000480, NT2RI2000575, NT2RI2000578, NT2RI2000588, NT2RI2000597, NT2RI2000669, NT2RI2000688, NT2RI2000704, NT2RI2000738, NT2RI2000775, NT2RI2000829, NT2RI2000841, NT2RI2000865, NT2RI2000932, NT2RI2000987, NT2RI2001010, NT2RI2001017,

NT2RI2001030, NT2RI2001054, NT2RI2001083, NT2RI2001091, NT2RI2001167, NT2RI2001178, NT2RI2001235, NT2RI2001244, NT2RI2001307, NT2RI2001385, NT2RI2001409, NT2RI2001410, NT2RI2001422, NT2RI2001425, NT2RI2001449, NT2RI2001450, NT2RI2001474, NT2RI2001519, NT2RI2001621, NT2RI2001624, NT2RI2001726, NT2RI2001731, NT2RI2001769, NT2RI2001782, NT2RI2001836, NT2RI2001846, NT2RI2001866, NT2RI2001910, NT2RI2001952, NT2RI2002022, NT2RI2002041, NT2RI2002117, NT2RI2002120, NT2RI2002152, NT2RI2002153, NT2RI2002179, NT2RI2002243, NT2RI2002252, NT2RI2002260, NT2RI2002266, NT2RI2002270, NT2RI2002334, NT2RI2002447, NT2RI2002481, NT2RI2002507, NT2RI2002517, NT2RI2002540, NT2RI2002549, NT2RI2002564, NT2RI2002585,

NT2RI2002592, NT2RI2002602, NT2RI2002802, NT2RI2002822, NT2RI2002852, NT2RI2002923, NT2RI2002926, NT2RI2002970, NT2RI2003011, NT2RI2003067, NT2RI2003184, NT2RI2003205, NT2RI2003222, NT2RI2003301, NT2RI2003344, NT2RI2003360, NT2RI2003361, NT2RI2003383, NT2RI2003392, NT2RI2003399, NT2RI2003419, NT2RI2003481, NT2RI2003487, NT2RI2003655, NT2RI2003667, NT2RI2003678, NT2RI2003751, NT2RI2003884, NT2RI2003973, NT2RI2004059, NT2RI2004078, NT2RI2004079, NT2RI2004093, NT2RI2004136, NT2RI2004157, NT2RI2004188, NT2RI2004190, NT2RI2004209, NT2RI2004284, NT2RI2004290, NT2RI2004304, NT2RI2004312, NT2RI2004381, NT2RI2004410, NT2RI2004442, NT2RI2004468, NT2RI2004535, NT2RI2004606, NT2RI2004618, NT2RI2004783,

NT2RI2004818, NT2RI2004840, NT2RI2004884, NT2RI2004916, NT2RI2004962, NT2RI2004984, NT2RI2004985, NT2RI2005048, NT2RI2005069, NT2RI2005087, NT2RI2005096, NT2RI2005130, NT2RI2005150, NT2RI2005166, NT2RI2005239, NT2RI2005246, NT2RI2005315, NT2RI2005358, NT2RI2005368, NT2RI2005382, NT2RI2005473, NT2RI2005520, NT2RI2005564, NT2RI2005579, NT2RI2005621, NT2RI2005628, NT2RI2005647, NT2RI2005670, NT2RI2005710, NT2RI2005713, NT2RI2005772, NT2RI2005816, NT2RI2005818, NT2RI2005851, NT2RI2006071, NT2RI2006072, NT2RI2006081, NT2RI2006126, NT2RI2006127, NT2RI2006183, NT2RI2006257, NT2RI2006271, NT2RI2006294, NT2RI2006345, NT2RI2006403, NT2RI2006412, NT2RI2006445, NT2RI2006487, NT2RI2006506, NT2RI2006640,

NT2RI2006659, NT2RI2006662, NT2RI2006667, NT2RI2006679, NT2RI2006703, NT2RI2006716, NT2RI2006735, NT2RI2006762, NT2RI2006788, NT2RI2006838, NT2RI2006853, NT2RI2006943, NT2RI2006973, NT2RI2007046, NT2RI2007048, NT2RI2007084, NT2RI2007096, NT2RI2007116, NT2RI2007148, NT2RI2007214, NT2RI2007277, NT2RI2007303, NT2RI2007334, NT2RI2007386, NT2RI2007406, NT2RI2007439, NT2RI2007445, NT2RI2007468, NT2RI2007469, NT2RI2007507, NT2RI2007572, NT2RI2007593, NT2RI2007629, NT2RI2007723, NT2RI2007729, NT2RI2007751, NT2RI2007754, NT2RI2007827, NT2RI2007877, NT2RI2007884, NT2RI2007891, NT2RI2007956, NT2RI2007965, NT2RI2008007, NT2RI2008045, NT2RI2008050, NT2RI2008171, NT2RI2008188, NT2RI2008204, NT2RI2008233,

NT2RI2008455, NT2RI2008481, NT2RI2008502, NT2RI2008566, NT2RI2008598, NT2RI2008622, NT2RI2008656, NT2RI2008714, NT2RI2008724, NT2RI2008801, NT2RI2008808, NT2RI2008841, NT2RI2008942, NT2RI2008952, NT2RI2009005, NT2RI2009066, NT2RI2009083, NT2RI2009101, NT2RI2009103, NT2RI2009144, NT2RI2009151, NT2RI2009173, NT2RI2009194, NT2RI2009215, NT2RI2009233, NT2RI2009259, NT2RI2009289, NT2RI2009301, NT2RI2009402, NT2RI2009406, NT2RI2009517, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012659, NT2RI2012726, NT2RI2013357, NT2RI2013373, NT2RI2014551, NT2RI2014683, NT2RI2014733, NT2RI2015239, NT2RI2016128,

NT2RI2016134, NT2RI2017529, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001445,

NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006376, NT2RI3006673, NT2RI3006796, NT2RI3007065, NT2RI3007095, NT2RI3007158, NT2RI3007167, NT2RI3007213, NT2RI3007291, NT2RI3007443, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697,

NT2RI3008974, NT2RI3009158, NT2RI3009480, NT2RI3009524, NT2RP6000008, NT2RP6000017, NT2RP6000033, NT2RP6000043, NT2RP6000059, NT2RP6000060, NT2RP6000085, NT2RP6000086, NT2RP6000100, NT2RP6000109, NT2RP6000119, NT2RP6000127, NT2RP6000130, NT2RP6000140, NT2RP6000150, NT2RP7000041, NT2RP7000086, NT2RP7000109, NT2RP7000112, NT2RP7000238, NT2RP7000259, NT2RP7000311, NT2RP7000359, NT2RP7000364, NT2RP7000380, NT2RP7000391, NT2RP7000425, NT2RP7000579, NT2RP7000586, NT2RP7000624, NT2RP7000812, NT2RP7000906, NT2RP7000926, NT2RP7001074, NT2RP7001080, NT2RP7001156, NT2RP7001231, NT2RP7001306, NT2RP7001319, NT2RP7001335, NT2RP7001364, NT2RP7001475, NT2RP7001532, NT2RP7001591, NT2RP7001602, NT2RP7001678,

NT2RP7001856, NT2RP7002064, NT2RP7002151, NT2RP7002163, NT2RP7002243, NT2RP7002282, NT2RP7002376, NT2RP7002449, NT2RP7002450, NT2RP7002478, NT2RP7002554, NT2RP7002594, NT2RP7002603, NT2RP7002650, NT2RP7002738, NT2RP7002779, NT2RP7002802, NT2RP7002829, NT2RP7002841, NT2RP7002875, NT2RP7002906, NT2RP7002978, NT2RP7003025, NT2RP7003050, NT2RP7003055, NT2RP7003084, NT2RP7003091, NT2RP7003107, NT2RP7003134, NT2RP7003148, NT2RP7003439, NT2RP7003511, NT2RP7003632, NT2RP7003680, NT2RP7003960, NT2RP7004027, NT2RP7004080, NT2RP7004123, NT2RP7004204, NT2RP7004233, NT2RP7004260, NT2RP7004348, NT2RP7004352, NT2RP7004358, NT2RP7004396, NT2RP7004428, NT2RP7004481, NT2RP7004541, NT2RP7004559, NT2RP7004641,

NT2RP7004656, NT2RP7004687, NT2RP7004722, NT2RP7004766, NT2RP7004790, NT2RP7004884, NT2RP7004911, NT2RP7004915, NT2RP7004961, NT2RP7004964, NT2RP7004975, NT2RP7005118, NT2RP7005205, NT2RP7005219, NT2RP7005323, NT2RP7005468, NT2RP7005493, NT2RP7005529, NT2RP7005629, NT2RP7005631, NT2RP7005669, NT2RP7005846, NT2RP7006160, NT2RP7006162, NT2RP7006223, NT2RP7006263, NT2RP7006296, NT2RP7006304, NT2RP7006374, NT2RP7006395, NT2RP7006408, NT2RP7006457, NT2RP7006490, NT2RP7006539, NT2RP7006547, NT2RP7006601, NT2RP7006621, NT2RP7006636, NT2RP7006701, NT2RP7006717, NT2RP7006853, NT2RP7006886, NT2RP7006887, NT2RP7006986, NT2RP7006995, NT2RP7007114, NT2RP7007221, NT2RP7007226, NT2RP7007252, NT2RP7007269,

NT2RP7007310, NT2RP7007359, NT2RP7007381, NT2RP7007406, NT2RP7007480, NT2RP7007504, NT2RP7007530, NT2RP7007580, NT2RP7007594, NT2RP7007610, NT2RP7007617, NT2RP7007643, NT2RP7007766, NT2RP7007842, NT2RP7007925, NT2RP7007930, NT2RP7008013, NT2RP7008015, NT2RP7008133, NT2RP7008137, NT2RP7008142, NT2RP7008144, NT2RP7008315, NT2RP7008360, NT2RP7008406, NT2RP7008435, NT2RP7008441, NT2RP7008454, NT2RP7008487, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009087, NT2RP7009108, NT2RP7009168, NT2RP7009215, NT2RP7009225, NT2RP7009236, NT2RP7009253, NT2RP7009267, NT2RP7009322,

NT2RP7009363, NT2RP7009370, NT2RP7009373, NT2RP7009397, NT2RP7009429, NT2RP7009439, NT2RP7009466, NT2RP7009481, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7015080,

NT2RP7015431, NT2RP7015503, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019135, NT2RP7019273, NT2RP7019367, NT2RP7019401, NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787,

NT2RP8004306, NT2RP8005546, NT2RP8006452, NT2RP8006521, NT2RP8007416, NT2RP8007503, NT2RP8007715, NT2RP8008057, NT2RP8009248, NTONG1000006, NTONG1000047, NTONG1000075, NTONG1000087, NTONG1000130, NTONG1000161, NTONG1000172, NTONG1000213, NTONG1000214, NTONG1000246, NTONG1000247, NTONG2000040, NTONG2000107, NTONG2000231, NTONG2000363, NTONG2000413, NTONG2000492, NTONG2000499, NTONG2000531, NTONG2000583, NTONG2000800, NTONG2000966, NTONG2001014, NTONG2001079, NTONG2001137, NTONG2001157, NTONG2001173, NTONG2001222, NTONG2001362, NTONG2001428, NTONG2001532, NTONG2001587, NTONG2001619, NTONG2001642, NTONG2001657, NTONG2001676, NTONG2001762, NTONG2002278, NTONG2002582, NTONG2002807, NTONG2002970,

NTONG2002985, NTONG2003158, NTONG2003210, NTONG2003316, NTONG2003409, NTONG2003515, NTONG2003805, NTONG2003839, NTONG2003897, NTONG2003928, NTONG2004095, NTONG2004308, NTONG2004521, NTONG2004690, NTONG2004806, NTONG2004829, NTONG2004918, NTONG2004991, NTONG2005062, NTONG2005086, NTONG2005137, NTONG2005153, NTONG2005265, NTONG2005277, NTONG2005363, NTONG2005373, NTONG2005480, NTONG2005577, NTONG2005657, NTONG2005822, NTONG2005897, NTONG2005969, NTONG2006099, NTONG2006187, NTONG2006301, NTONG2006324, NTONG2006354, NTONG2006440, NTONG2006501, NTONG2006646, NTONG2006709, NTONG2007020, NTONG2007028, NTONG2007249, NTONG2007517, NTONG2007522, NTONG2007693, NTONG2008088, NTONG2008093, NTONG2008143,

NTONG2008365, NTONG2008522, NTONG2008672, NTONG2008862, NTONG2008939, NTONG2009068, NTONG2009229, NTONG2009233, NTONG2009468, NTONG2009576, OCBBF1000016, OCBBF1000030, OCBBF1000042, OCBBF1000049, OCBBF1000054, OCBBF1000067, OCBBF1000080, OCBBF1000085, OCBBF1000086, OCBBF1000091, OCBBF1000104, OCBBF1000114, OCBBF1000118, OCBBF1000119, OCBBF1000122, OCBBF1000130, OCBBF1000138, OCBBF1000145, OCBBF1000153, OCBBF1000175, OCBBF1000185, OCBBF1000192, OCBBF1000254, OCBBF2000126, OCBBF2000178, OCBBF2000231, OCBBF2000277, OCBBF2000287, OCBBF2000323, OCBBF2000452, OCBBF2000467, OCBBF2000522, OCBBF2000677, OCBBF2000703, OCBBF2000712, OCBBF2000719, OCBBF2000831, OCBBF2000904, OCBBF2000982, OCBBF2000998,

OCBBF2001075, OCBBF2001101, OCBBF2001124, OCBBF2001140, OCBBF2001166, OCBBF2001176, OCBBF2001186, OCBBF2001196, OCBBF2001252, OCBBF2001323, OCBBF2001389, OCBBF2001402, OCBBF2001408, OCBBF2001416, OCBBF2001473, OCBBF2001494, OCBBF2001527, OCBBF2001528, OCBBF2001681, OCBBF2001706, OCBBF2001749, OCBBF2001794, OCBBF2001910, OCBBF2001931, OCBBF2001961, OCBBF2002015, OCBBF2002083, OCBBF2002124, OCBBF2002161, OCBBF2002290, OCBBF2002357, OCBBF2002376, OCBBF2002615, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003543, OCBBF2003593, OCBBF2003744, OCBBF2003819, OCBBF2003925, OCBBF2004038,

OCBBF2004104, OCBBF2004147, OCBBF2004168, OCBBF2004235, OCBBF2004259, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843, OCBBF2005901, OCBBF2006030, OCBBF2006058, OCBBF2006113, OCBBF2006151, OCBBF2006154, OCBBF2006172, OCBBF2006214, OCBBF2006241, OCBBF2006313, OCBBF2006388, OCBBF2006438, OCBBF2006567, OCBBF2006624, OCBBF2006639, OCBBF2006660, OCBBF2006764,

OCBBF2006859, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007414, OCBBF2007415, OCBBF2007428, OCBBF2007478, OCBBF2007485, OCBBF2007520, OCBBF2007555, OCBBF2007610, OCBBF2007756, OCBBF2007762, OCBBF2007892, OCBBF2007931, OCBBF2007946, OCBBF2008005, OCBBF2008041, OCBBF2008116, OCBBF2008138, OCBBF2008144, OCBBF2008283, OCBBF2008330, OCBBF2008340, OCBBF2008366, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770, OCBBF2008775,

OCBBF2008790, OCBBF2008814, OCBBF2008822, OCBBF2009115, OCBBF2009242, OCBBF2009301, OCBBF2009352, OCBBF2009391, OCBBF2009424, OCBBF2009536, OCBBF2009571, OCBBF2009583, OCBBF2009788, OCBBF2009836, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010830, OCBBF2010841, OCBBF2010843, OCBBF2010858, OCBBF2010863, OCBBF2010871, OCBBF2010931, OCBBF2010945, OCBBF2010978, OCBBF2010989, OCBBF2011021, OCBBF2011073, OCBBF2011137, OCBBF2011160, OCBBF2011177, OCBBF2011228, OCBBF2011232, OCBBF2011283, OCBBF2011311, OCBBF2011536, OCBBF2011625, OCBBF2011669,

OCBBF2011685, OCBBF2011706, OCBBF2011722, OCBBF2011759, OCBBF2011767, OCBBF2011855, OCBBF2011872, OCBBF2011897, OCBBF2011914, OCBBF2011960, OCBBF2011981, OCBBF2012001, OCBBF2012028, OCBBF2012039, OCBBF2012139, OCBBF2012191, OCBBF2012262, OCBBF2012320, OCBBF2012436, OCBBF2012525, OCBBF2012553, OCBBF2012678, OCBBF2012704, OCBBF2012755, OCBBF2012812, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789, OCBBF2013843, OCBBF2013883, OCBBF2013926, OCBBF2014052, OCBBF2014064, OCBBF2014089,

OCBBF2014292, OCBBF2014322, OCBBF2014386, OCBBF2014541, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016841, OCBBF2017069, OCBBF2017306, OCBBF2017325, OCBBF2017489, OCBBF2017516, OCBBF2017665, OCBBF2017815, OCBBF2017882, OCBBF2017893, OCBBF2017920, OCBBF2018014, OCBBF2018084, OCBBF2018167, OCBBF2018206,

OCBBF2018229, OCBBF2018234, OCBBF2018362, OCBBF2018380, OCBBF2018563, OCBBF2018581, OCBBF2018618, OCBBF2018663, OCBBF2018687, OCBBF2018707, OCBBF2018827, OCBBF2018828, OCBBF2018934, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021020, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2025028, OCBBF2025115, OCBBF2025198, OCBBF2025451,

OCBBF2025458, OCBBF2025527, OCBBF2025631, OCBBF2025730, OCBBF2026025, OCBBF2026035, OCBBF2026144, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948,

OCBBF2034333, OCBBF2034529, OCBBF2034637, OCBBF2034906, OCBBF2035110, OCBBF2035214, OCBBF2035226, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004972,

OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, SALGL1000024, SALGL1000028, SALGL1000065, SALGL1000107, SALGL1000171, SKMUS1000014, SKMUS1000030, SKMUS1000067, SKMUS1000102, SKMUS1000104, SKMUS1000110, SKMUS1000118, SKMUS1000124, SKMUS1000129, SKMUS1000176, SKMUS1000177, SKMUS2000020, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000343, SKMUS2000365, SKMUS2000390, SKMUS2000416, SKMUS2000467, SKMUS2000468, SKMUS2000484, SKMUS2000690,

SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000894, SKMUS2000898, SKMUS2000902, SKMUS2000933, SKMUS2000945, SKMUS2000954, SKMUS2000961, SKMUS2000973, SKMUS2000980, SKMUS2001014, SKMUS2001164, SKMUS2001199, SKMUS2001215, SKMUS2001247, SKMUS2001254, SKMUS2001271, SKMUS2001287, SKMUS2001323, SKMUS2001364, SKMUS2001398, SKMUS2001412, SKMUS2001492, SKMUS2001501, SKMUS2001528, SKMUS2001543, SKMUS2001580, SKMUS2001585, SKMUS2001608, SKMUS2001631, SKMUS2001662, SKMUS2001663, SKMUS2001671, SKMUS2001683, SKMUS2001703, SKMUS2001740, SKMUS2001742, SKMUS2001823, SKMUS2001850, SKMUS2002077,

SKMUS2002084, SKMUS2002153, SKMUS2002475, SKMUS2002602, SKMUS2002693, SKMUS2002821, SKMUS2002840, SKMUS2002920, SKMUS2003034, SKMUS2003074, SKMUS2003194, SKMUS2003744, SKMUS2004044, SKMUS2004047, SKMUS2004483, SKMUS2004897, SKMUS2004908, SKMUS2005428, SKMUS2006394, SKMUS2006481, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000110, SKNMC1000123, SKNMC1000124, SKNMC1000159, SKNMC1000229, SKNMC1000251, SKNMC1000264, SKNMC2000065, SKNMC2000224, SKNMC2000256, SKNMC2000305, SKNMC2000356, SKNMC2000374, SKNMC2000583, SKNMC2000593,

SKNMC2000612, SKNMC2000622, SKNMC2001113, SKNMC2001324, SKNMC2001503, SKNMC2001555, SKNMC2001596, SKNMC2001623, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000086, SKNSH1000174, SKNSH1000301, SKNSH1000308, SKNSH1000416, SKNSH2000051, SKNSH2000101, SKNSH2000151, SKNSH2000163, SKNSH2000250, SKNSH2000319, SKNSH2000482, SKNSH2000516, SKNSH2000550, SKNSH2000716, SKNSH2000819, SKNSH2000971, SKNSH2001222, SKNSH2001875, SKNSH2001931, SKNSH2002054, SKNSH2002325, SKNSH2002866, SKNSH2003064, SKNSH2003174, SKNSH2003422, SKNSH2003466,

SKNSH2003483, SKNSH2003633, SKNSH2004039, SKNSH2005061, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008043, SKNSH2008969, SKNSH2009197, SKNSH2009357, SKNSH2009435, SKNSH2009991, SKNSH2010015, SMINT1000012, SMINT1000016, SMINT1000039, SMINT1000048, SMINT1000051, SMINT1000054, SMINT1000057, SMINT1000071, SMINT1000075, SMINT1000091, SMINT1000103, SMINT1000118, SMINT1000137, SMINT1000192, SMINT2000007, SMINT2000032, SMINT2000040, SMINT2000060, SMINT2000073, SMINT2000084, SMINT2000145, SMINT2000159, SMINT2000176, SMINT2000185, SMINT2000227,

SMINT2000229, SMINT2000232, SMINT2000239, SMINT2000267, SMINT2000400, SMINT2000454, SMINT2000518, SMINT2000529, SMINT2000530, SMINT2000537, SMINT2000538, SMINT2000541, SMINT2000558, SMINT2000562, SMINT2000568, SMINT2000609, SMINT2000629, SMINT2000659, SMINT2000694, SMINT2000747, SMINT2000811, SMINT2000824, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001950, SMINT2002126, SMINT2002159, SMINT2002202, SMINT2002210, SMINT2002224, SMINT2002281, SMINT2002311, SMINT2002314, SMINT2002328, SMINT2002414,

SMINT2002457, SMINT2002743, SMINT2002778, SMINT2002880, SMINT2002882, SMINT2002884, SMINT2002899, SMINT2002974, SMINT2003003, SMINT2003074, SMINT2003128, SMINT2003150, SMINT2003169, SMINT2003317, SMINT2003340, SMINT2003386, SMINT2003423, SMINT2003551, SMINT2003569, SMINT2003641, SMINT2003644, SMINT2003866, SMINT2003905, SMINT2004037, SMINT2004047, SMINT2004086, SMINT2004144, SMINT2004280, SMINT2004299, SMINT2004339, SMINT2004350, SMINT2004414, SMINT2004473, SMINT2004547, SMINT2004583, SMINT2004589, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005368, SMINT2005387,

SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006716, SMINT2006801, SMINT2007062, SMINT2007101, SMINT2007140, SMINT2007187, SMINT2007219, SMINT2007294, SMINT2007365, SMINT2007391, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008625, SMINT2008672, SMINT2008690, SMINT2008715, SMINT2008720,

SMINT2008817, SMINT2008844, SMINT2008869, SMINT2009119, SMINT2009121, SMINT2009212, SMINT2009233, SMINT2009259, SMINT2009272, SMINT2009292, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010144, SMINT2010200, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010959, SMINT2010997, SMINT2011033, SMINT2011066, SMINT2011273, SMINT2011406, SMINT2011509, SMINT2011588, SMINT2011656, SMINT2011888, SMINT2011958, SMINT2012040, SMINT2012179,

SMINT2012195, SMINT2012285, SMINT2012291, SMINT2012501, SMINT2012734, SMINT2012793, SMINT2013031, SMINT2013032, SMINT2013129, SMINT2013181, SMINT2013228, SMINT2013613, SMINT2013626, SMINT2013695, SMINT2013833, SMINT2013890, SMINT2014166, SMINT2014282, SMINT2014443, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659,

SMINT2017736, SMINT2017781, SMINT2017855, SMINT2017884, SMINT2017964, SMINT2017974, SMINT2018343, SMINT2018353, SMINT2018592, SMINT2019017, SMINT2019105, SMINT2019142, SMINT2019153, SMINT2019200, SPLEN1000041, SPLEN1000049, SPLEN1000056, SPLEN1000087, SPLEN1000089, SPLEN1000091, SPLEN1000106, SPLEN1000116, SPLEN1000134, SPLEN1000143, SPLEN1000144, SPLEN1000145, SPLEN1000156, SPLEN1000163, SPLEN1000165, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000210, SPLEN2000242, SPLEN2000243, SPLEN2000247, SPLEN2000255, SPLEN2000258, SPLEN2000267, SPLEN2000270, SPLEN2000307,

SPLEN2000310, SPLEN2000341, SPLEN2000350, SPLEN2000357, SPLEN2000364, SPLEN2000402, SPLEN2000414, SPLEN2000420, SPLEN2000448, SPLEN2000450, SPLEN2000464, SPLEN2000496, SPLEN2000505, SPLEN2000515, SPLEN2000516, SPLEN2000537, SPLEN2000541, SPLEN2000607, SPLEN2000630, SPLEN2000695, SPLEN2000698, SPLEN2000839, SPLEN2000874, SPLEN2001135, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001689, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175,

SPLEN2002223, SPLEN2002272, SPLEN2002314, SPLEN2002343, SPLEN2002354, SPLEN2002385, SPLEN2002419, SPLEN2002451, SPLEN2002462, SPLEN2002463, SPLEN2002662, SPLEN2002695, SPLEN2002707, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004220, SPLEN2004343, SPLEN2004346, SPLEN2004368, SPLEN2004555, SPLEN2004611, SPLEN2004662, SPLEN2004773, SPLEN2004844, SPLEN2004880, SPLEN2004984, SPLEN2005009, SPLEN2005227, SPLEN2005272, SPLEN2005342, SPLEN2005416, SPLEN2005450, SPLEN2005474, SPLEN2005560, SPLEN2005727,

SPLEN2005783, SPLEN2005790, SPLEN2005806, SPLEN2005818, SPLEN2005838, SPLEN2005869, SPLEN2005927, SPLEN2005939, SPLEN2006143, SPLEN2006268, SPLEN2006283, SPLEN2006325, SPLEN2006374, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007498, SPLEN2007619, SPLEN2007653, SPLEN2007689, SPLEN2007695, SPLEN2007879, SPLEN2007926, SPLEN2007951, SPLEN2008007, SPLEN2008135, SPLEN2008164, SPLEN2008317, SPLEN2008496, SPLEN2008591, SPLEN2008786, SPLEN2009081, SPLEN2009088, SPLEN2009163, SPLEN2009315, SPLEN2009340, SPLEN2009512, SPLEN2009513, SPLEN2009541, SPLEN2009548, SPLEN2009555,

SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011678, SPLEN2011758, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234,

SPLEN2012453, SPLEN2012524, SPLEN2012525, SPLEN2012571, SPLEN2012606, SPLEN2012611, SPLEN2012619, SPLEN2012624, SPLEN2012725, SPLEN2012743, SPLEN2012756, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014080, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014572, SPLEN2014645, SPLEN2014669, SPLEN2014711, SPLEN2014739, SPLEN2014860,

SPLEN2014865, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2615166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016304, SPLEN2016356, SPLEN2016421, SPLEN2016480, SPLEN2016531, SPLEN2016533, SPLEN2016541, SPLEN2016552, SPLEN2016602, SPLEN2016627, SPLEN2016678, SPLEN2016743,

SPLEN2016773, SPLEN2016781, SPLEN2016840, SPLEN2016863, SPLEN2016922, SPLEN2016932, SPLEN2016972, SPLEN2016973, SPLEN2017043, SPLEN2017104, SPLEN2017121, SPLEN2017147, SPLEN2017187, SPLEN2017189, SPLEN2017212, SPLEN2017318, SPLEN2017351, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017999, SPLEN2018098, SPLEN2018112, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019092, SPLEN2019102, SPLEN2019131, SPLEN2019169,

SPLEN2019257, SPLEN2019311, SPLEN2019323, SPLEN2019349, SPLEN2019375, SPLEN2019379, SPLEN2019405, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021194, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701,

SPLEN2021795, SPLEN2021983, SPLEN2021991, SPLEN2022040, SPLEN2022162, SPLEN2022227, SPLEN2022522, SPLEN2022591, SPLEN2022785, SPLEN2022920, SPLEN2023024, SPLEN2023118, SPLEN2023160, SPLEN2023423, SPLEN2023710, SPLEN2023733, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066,

SPLEN2028138, SPLEN2028365, SPLEN2028417, SPLEN2028424, SPLEN2028593, SPLEN2028844, SPLEN2028994, SPLEN2029051, SPLEN2029176, SPLEN2029295, SPLEN2029380, SPLEN2029522, SPLEN2029683, SPLEN2029727, SPLEN2029904, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031547, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551,

SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036953, SPLEN2037077, SPLEN2037194, SPLEN2037319, SPLEN2037444, SPLEN2037580, SPLEN2037630, SPLEN2037678, SPLEN2037891, SPLEN2038055, SPLEN2038180, SPLEN2038345, SPLEN2038407, SPLEN2038473, SPLEN2039311, SPLEN2039379, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303,

SPLEN2042521, SPLEN2042598, SPLEN2042714, STOMA1000013, STOMA1000047, STOMA1000052, STOMA1000074, STOMA1000091, STOMA1000117, STOMA1000133, STOMA1000175, STOMA1000186, STOMA1000190, STOMA1000191, STOMA2000032, STOMA2000072, STOMA2000088, STOMA2000104, STOMA2000148, STOMA2000168, STOMA2000183, STOMA2000188, STOMA2000279, STOMA2000289, STOMA2000383, STOMA2000386, STOMA2000395, STOMA2000396, STOMA2000478, STOMA2000529, STOMA2000539, STOMA2000567, STOMA2000640, STOMA2000678, STOMA2000686, STOMA2000888, STOMA2001025, STOMA2001389, STOMA2002052, STOMA2002141, STOMA2002367, STOMA2002688, STOMA2002828, STOMA2003158, STOMA2003289, STOMA2003444, STOMA2003477, STOMA2003646, STOMA2003716, STOMA2003781,

STOMA2003894, STOMA2004194, STOMA2004294, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213, STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291,

SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001144, SYNOV2001212, SYNOV2001251, SYNOV2001300, SYNOV2001356, SYNOV2001390, SYNOV2001451, SYNOV2001457, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017055, SYNOV2017179, SYNOV2018921, SYNOV2019280, SYNOV2020085, SYNOV2020463, SYNOV2021320,

SYNOV2021953, SYNOV3000184, SYNOV3000231, SYNOV3000302, SYNOV3000345, SYNOV4000249, SYNOV4000472, SYNOV4000598, SYNOV4000706, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007711, SYNOV4008245, SYNOV4008336, SYNOV4009129, SYNOV4009139, SYNOV4009298, T1ESE2000116, T1ESE2000609, T1ESE2000904, TBAES2000004,

TBAES2000059, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000127, TESOP1000160,

TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150, TESOP2002451, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006060, TESOP2006068, TESOP2006110, TESOP2006398, TESOP2006670, TESOP2006704,

TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000055, TESTI1000070, TESTI1000085, TESTI1000088, TESTI1000093, TESTI1000094, TESTI1000096, TESTI1000109, TESTI1000125, TESTI1000127, TESTI1000128, TESTI1000131, TESTI1000138, TESTI1000157, TESTI1000168, TESTI1000169, TESTI1000179, TESTI1000183, TESTI1000191, TESTI1000257, TESTI1000266, TESTI1000319,

TESTI1000330, TESTI1000348, TESTI1000390, TESTI1000391, TESTI1000459, TESTI1000491, TESTI1000545, TESTI2000018, TESTI2000044, TESTI2000100, TESTI2000117, TESTI2000120, TESTI2000147, TESTI2000154, TESTI2000177, TESTI2000179, TESTI2000207, TESTI2000208, TESTI2000221, TESTI2000238, TESTI2000253, TESTI2000272, TESTI2000278, TESTI2000356, TESTI2000358, TESTI2000372, TESTI2000427, TESTI2000431, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000808,

TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082, TESTI2001134, TESTI2001141, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815,

TESTI2001823, TESTI2001826, TESTI2001827, TESTI2001852, TESTI2001862, TESTI2001869, TESTI2001879, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002149, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618,

TESTI2002632, TESTI2002676, TESTI2002690, TESTI2002696, TESTI2002698, TESTI2002700, TESTI2002707, TESTI2002724, TESTI2002729, TESTI2002752, TESTI2002753, TESTI2002789, TESTI2002802, TESTI2002806, TESTI2002840, TESTI2002866, TESTI2002877, TESTI2002910, TESTI2002912, TESTI2002928, TESTI2002937, TESTI2002965, TESTI2002993, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280,

TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003418, TESTI2003419, TESTI2003432, TESTI2003454, TESTI2003475, TESTI2003476, TESTI2003498, TESTI2003512, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004080,

TESTI2004085, TESTI2004089, TESTI2004111, TESTI2004119, TESTI2004122, TESTI2004163, TESTI2004169, TESTI2004207, TESTI2004215, TESTI2004227, TESTI2004229, TESTI2004243, TESTI2004255, TESTI2004287, TESTI2004295, TESTI2004313, TESTI2004318, TESTI2004322, TESTI2004391, TESTI2004399, TESTI2004423, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004737, TESTI2004783, TESTI2004791, TESTI2004793, TESTI2004860, TESTI2004906, TESTI2004920, TESTI2004936, TESTI2004941, TESTI2004971,

TESTI2004982, TESTI2004993, TESTI2004994, TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415, TESTI2005470, TESTI2005492, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005743, TESTI2005759, TESTI2005767,

TESTI2005775, TESTI2005827, TESTI2005835, TESTI2005860, TESTI2005892, TESTI2005908, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006457, TESTI2006460, TESTI2006465,

TESTI2006483, TESTI2006499, TESTI2006523, TESTI2006543, TESTI2006565, TESTI2006572, TESTI2006615, TESTI2006617, TESTI2006624, TESTI2006643, TESTI2006648, TESTI2006659, TESTI2006660, TESTI2006665, TESTI2006667, TESTI2006677, TESTI2006720, TESTI2006735, TESTI2006744, TESTI2006774, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007311, TESTI2007320, TESTI2007346, TESTI2007358, TESTI2007402, TESTI2007405, TESTI2007422, TESTI2007452, TESTI2007464,

TESTI2007466, TESTI2007480, TESTI2007490, TESTI2007502, TESTI2007524, TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008320, TESTI2008331, TESTI2008343, TESTI2008387, TESTI2008389, TESTI2008394, TESTI2008405, TESTI2008420, TESTI2008425, TESTI2008440,

TESTI2008567, TESTI2008595, TESTI2008636, TESTI2008657, TESTI2008684, TESTI2008699, TESTI2008715, TESTI2008762, TESTI2008774, TESTI2008786, TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009477, TESTI2009497, TESTI2009502, TESTI2009511, TESTI2009544, TESTI2009545, TESTI2009551, TESTI2009585, TESTI2009588, TESTI2009683, TESTI2009727, TESTI2009739, TESTI2009812, TESTI2009835,

TESTI2009853, TESTI2009935, TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011680, TESTI2011683, TESTI2011712, TESTI2011750, TESTI2011840, TESTI2011846,

TESTI2011915, TESTI2011928, TESTI2011971, TESTI2012019, TESTI2012050, TESTI2012090, TESTI2012095, TESTI2012104, TESTI2012152, TESTI2012171, TESTI2012207, TESTI2012231, TESTI2012244, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013257, TESTI2013264, TESTI2013268, TESTI2013295, TESTI2013322, TESTI2013381, TESTI2013382, TESTI2013401, TESTI2013427, TESTI2013468,

TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014838, TESTI2014843, TESTI2014979, TESTI2014988, TESTI2015042, TESTI2015105, TESTI2015180, TESTI2015246, TESTI2015249, TESTI2015327, TESTI2015331, TESTI2015335, TESTI2015350,

TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113,

TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018014, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018867, TESTI2018941, TESTI2018957, TESTI2018974, TESTI2018975, TESTI2018997, TESTI2019042, TESTI2019077, TESTI2019228,

TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020515, TESTI2020525, TESTI2020579, TESTI2020717, TESTI2020840, TESTI2020871, TESTI2020905, TESTI2020906, TESTI2020956, TESTI2020981, TESTI2020999,

TESTI2021003, TESTI2021038, TESTI2021057, TESTI2021077, TESTI2021112, TESTI2021114, TESTI2021116, TESTI2021118, TESTI2021122, TESTI2021124, TESTI2021138, TESTI2021358, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025,

TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024864, TESTI2024885, TESTI2024936, TESTI2024978, TESTI2024999, TESTI2025006, TESTI2025013, TESTI2025022, TESTI2025063,

TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025454, TESTI2025486, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605,

TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583,

TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919,

TESTI2031935, TESTI2031986, TESTI2032044, TESTI2032067, TESTI2032079, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183,

TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877,

TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983,

TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044306, TESTI2044309, TESTI2044413, TESTI2044418, TESTI2044754, TESTI2044788,

TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047605, TESTI2047792, TESTI2047801, TESTI2047818, TESTI2047824, TESTI2047885, TESTI2047930, TESTI2048105, TESTI2048109,

TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667,

TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001473, TESTI4001517, TESTI4001527, TESTI4001561, TESTI4001569, TESTI4001604, TESTI4001665, TESTI4001679,

TESTI4001795, TESTI4001923, TESTI4001925, TESTI4001984, TESTI4002003, TESTI4002141, TESTI4002195, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317, TESTI4005322, TESTI4005399,

TESTI4005470, TESTI4005500, TESTI4005534, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965, TESTI4008007,

TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789,

TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013602, TESTI4013667, TESTI4013675, TESTI4013685, TESTI4013735, TESTI4013742, TESTI4013774, TESTI4013817, TESTI4013830, TESTI4013852, TESTI4013894,

TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229,

TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491,

TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762,

TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270,

TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038339,

TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043140, TESTI4043166, TESTI4043203, TESTI4043223, TESTI4043371,

TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000017,

THYMU1000025, THYMU1000029, THYMU1000033, THYMU1000041, THYMU1000060, THYMU1000074, THYMU1000098, THYMU1000103, THYMU1000105, THYMU1000109, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000329, THYMU1000331, THYMU1000336, THYMU1000366, THYMU1000374, THYMU1000382, THYMU1000393, THYMU1000399, THYMU1000426, THYMU1000440, THYMU1000459, THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022,

THYMU2000032, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000436, THYMU2000440, THYMU2000489, THYMU2000492, THYMU2000519, THYMU2000570, THYMU2000602, THYMU2000660, THYMU2000671, THYMU2000672, THYMU2000702, THYMU2000767, THYMU2000775, THYMU2000800, THYMU2000879, THYMU2000932, THYMU2000946, THYMU2000950, THYMU2000971, THYMU2000982, THYMU2000990, THYMU2001007, THYMU2001018, THYMU2001053, THYMU2001071, THYMU2001090, THYMU2001139, THYMU2001195, THYMU2001202, THYMU2001203, THYMU2001256, THYMU2001270,

THYMU2001291, THYMU2001314, THYMU2001325, THYMU2001357, THYMU2001381, THYMU2001422, THYMU2001443, THYMU2001515, THYMU2001556, THYMU2001622, THYMU2001727, THYMU2001819, THYMU2001825, THYMU2001839, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002162, THYMU2002177, THYMU2002203, THYMU2002242, THYMU2002338, THYMU2002356, THYMU2002376, THYMU2002384, THYMU2002450, THYMU2002490, THYMU2002548, THYMU2002695, THYMU2002756, THYMU2002789, THYMU2002792, THYMU2002815, THYMU2002852, THYMU2002910, THYMU2002950, THYMU2002983, THYMU2003107, THYMU2003124, THYMU2003166,

THYMU2003232, THYMU2003242, THYMU2003249, THYMU2003270, THYMU2003282, THYMU2003287, THYMU2003336, THYMU2003366, THYMU2003397, THYMU2003440, THYMU2003446, THYMU2003470, THYMU2003479, THYMU2003499, THYMU2003542, THYMU2003632, THYMU2003650, THYMU2003700, THYMU2003760, THYMU2003803, THYMU2003855, THYMU2003907, THYMU2003932, THYMU2004003, THYMU2004008, THYMU2004042, THYMU2004111, THYMU2004123, THYMU2004139, THYMU2004169, THYMU2004284, THYMU2004336, THYMU2004356, THYMU2004452, THYMU2004508, THYMU2004512, THYMU2004635, THYMU2004639, THYMU2004668, THYMU2004677, THYMU2004688, THYMU2004693, THYMU2004835, THYMU2004843, THYMU2004906, THYMU2004916, THYMU2004986, THYMU2005001, THYMU2005003, THYMU2005046,

THYMU2005134, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006913, THYMU2006946, THYMU2006965, THYMU2007025, THYMU2007060, THYMU2007095,

THYMU2007146, THYMU2007179, THYMU2007258, THYMU2007307, THYMU2007308, THYMU2007339, THYMU2007350, THYMU2007384, THYMU2007415, THYMU2007467, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452, THYMU2008643, THYMU2008686, THYMU2008691, THYMU2008714,

THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009835, THYMU2009906, THYMU2009948, THYMU2010030, THYMU2010041, THYMU2010046, THYMU2010059, THYMU2010082, THYMU2010161, THYMU2010192, THYMU2010448, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072, THYMU2011096, THYMU2011118, THYMU2011142,

THYMU2011183, THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011937, THYMU2011939, THYMU2012002, THYMU2012073, THYMU2012104, THYMU2012113, THYMU2012273, THYMU2012401, THYMU2012492, THYMU2012507, THYMU2012565, THYMU2012574, THYMU2012631, THYMU2012665, THYMU2012690, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013170, THYMU2013364,

THYMU2013386, THYMU2013426, THYMU2013578, THYMU2013705, THYMU2013757, THYMU2013779, THYMU2013863, THYMU2013950, THYMU2013981, THYMU2014067, THYMU2014068, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015161, THYMU2015316, THYMU2015321, THYMU2015439, THYMU2015441, THYMU2015479, THYMU2015762, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099,

THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830,

THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894,

THYMU2027996, THYMU2028041, THYMU2028368, THYMU2028379, THYMU2028394, THYMU2028412, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031368, THYMU2031579, THYMU2031847, THYMU2031890, THYMU2031994, THYMU2032014, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976,

THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2036058, THYMU2036105, THYMU2036207, THYMU2036215, THYMU2036252, THYMU2036265, THYMU2036366, THYMU2036459, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189,

THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841,

THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137,

THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015962,

THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024602, THYMU3024879, THYMU3025118, THYMU3025313, THYMU3025683, THYMU3025772, THYMU3025865, THYMU3026000, THYMU3026350, THYMU3026479, THYMU3026532, THYMU3026783, THYMU3026869,

THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032798, THYMU3032867, THYMU3033402, THYMU3033626, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827,

THYMU3037836, THYMU3037856, THYMU3037867, THYMU3037909, THYMU3037980, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175,

THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000171, TKIDN1000192, TKIDN2000127, TKIDN2000240, TKIDN2000246, TKIDN2000319, TKIDN2000464, TKIDN2000521, TKIDN2000701, TKIDN2001529, TKIDN2002318, TKIDN2002329, TKIDN2002424, TKIDN2002632, TKIDN2003044, TKIDN2003059, TKIDN2003062, TKIDN2003078, TKIDN2003366, TKIDN2003396, TKIDN2003413, TKIDN2003601, TKIDN2004185, TKIDN2004386, TKIDN2004458, TKIDN2004748, TKIDN2005487, TKIDN2005721, TKIDN2005934,

TKIDN2005947, TKIDN2005956, TKIDN2006525, TKIDN2006761, TKIDN2006852, TKIDN2007605, TKIDN2007667, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616,

TLIVE2001684, TLIVE2001828, TLIVE2001927, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575,

TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000057, TRACH1000074, TRACH1000100, TRACH1000125, TRACH1000140, TRACH1000144, TRACH1000181, TRACH1000193, TRACH1000212, TRACH1000240, TRACH2000002, TRACH2000015, TRACH2000024, TRACH2000032, TRACH2000045, TRACH2000047, TRACH2000083, TRACH2000148, TRACH2000185, TRACH2000196, TRACH2000235, TRACH2000237, TRACH2000243, TRACH2000250, TRACH2000259, TRACH2000272, TRACH2000282, TRACH2000287, TRACH2000289, TRACH2000306, TRACH2000312, TRACH2000336, TRACH2000359, TRACH2000393, TRACH2000411, TRACH2000461, TRACH2000496, TRACH2000497, TRACH2000502,

TRACH2000522, TRACH2000540, TRACH2000559, TRACH2000660, TRACH2000665, TRACH2000675, TRACH2000677, TRACH2000703, TRACH2000767, TRACH2000775, TRACH2000780, TRACH2000807, TRACH2000862, TRACH2000873, TRACH2000894, TRACH2000898, TRACH2000920, TRACH2000923, TRACH2000926, TRACH2000944, TRACH2000981, TRACH2000986, TRACH2001037, TRACH2001060, TRACH2001085, TRACH2001109, TRACH2001121, TRACH2001147, TRACH2001154, TRACH2001188, TRACH2001192, TRACH2001249, TRACH2001275, TRACH2001335, TRACH2001339, TRACH2001341, TRACH2001347, TRACH2001395, TRACH2001400, TRACH2001403, TRACH2001416, TRACH2001428, TRACH2001432, TRACH2001474, TRACH2001525, TRACH2001532, TRACH2001549, TRACH2001592, TRACH2001596, TRACH2001621,

TRACH2001646, TRACH2001651, TRACH2001684, TRACH2001810, TRACH2001933, TRACH2001968, TRACH2001996, TRACH2002043, TRACH2002054, TRACH2002100, TRACH2002138, TRACH2002333, TRACH2002784, TRACH2002803, TRACH2002840, TRACH2002853, TRACH2002862, TRACH2002954, TRACH2002960, TRACH2002988, TRACH2003070, TRACH2003272, TRACH2003347, TRACH2003484, TRACH2003516, TRACH2003605, TRACH2003736, TRACH2003759, TRACH2003941, TRACH2004039, TRACH2004054, TRACH2004087, TRACH2004109, TRACH2004170, TRACH2004183, TRACH2004251, TRACH2004292, TRACH2004336, TRACH2004399, TRACH2004428, TRACH2004458, TRACH2004521, TRACH2004570, TRACH2004845, TRACH2004887, TRACH2004950, TRACH2005004, TRACH2005017, TRACH2005066, TRACH2005159,

TRACH2005314, TRACH2005444, TRACH2005541, TRACH2005666, TRACH2005698, TRACH2005710, TRACH2005720, TRACH2005769, TRACH2005796, TRACH2005800, TRACH2006015, TRACH2006049, TRACH2006097, TRACH2006194, TRACH2006327, TRACH2006331, TRACH2006378, TRACH2006387, TRACH2006528, TRACH2006670, TRACH2006866, TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007894, TRACH2007969, TRACH2007988, TRACH2008081, TRACH2008112, TRACH2008113, TRACH2008127, TRACH2008130, TRACH2008278, TRACH2008300, TRACH2008478, TRACH2008540, TRACH2008583, TRACH2008597, TRACH2008716, TRACH2008820, TRACH2008921,

TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011113, TRACH2011290, TRACH2011293, TRACH2011302, TRACH2011574, TRACH2011894, TRACH2011956, TRACH2012000, TRACH2012138, TRACH2012242, TRACH2012298, TRACH2012370, TRACH2012376, TRACH2012387,

TRACH2012497, TRACH2012562, TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2014018, TRACH2014045, TRACH2014046, TRACH2014077, TRACH2014082, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015987,

TRACH2016080, TRACH2016131, TRACH2016286, TRACH2016317, TRACH2016347, TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017498, TRACH2017609, TRACH2017949, TRACH2017965, TRACH2017980, TRACH2018034, TRACH2018084, TRACH2018262, TRACH2018273, TRACH2018278, TRACH2018345, TRACH2018380, TRACH2018449, TRACH2018512, TRACH2018646, TRACH2018663, TRACH2018718, TRACH2018835, TRACH2018882, TRACH2018914, TRACH2018950, TRACH2019024, TRACH2019046, TRACH2019080, TRACH2019151, TRACH2019154, TRACH2019176, TRACH2019248, TRACH2019309, TRACH2019425,

TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025535,

TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003586, TRACH3003683, TRACH3003780,

TRACH3003805, TRACH3003832, TRACH3003872, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005211, TRACH3005274, TRACH3005294, TRACH3005479, TRACH3005549, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699,

TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007627, TRACH3007689, TRACH3007766, TRACH3007866, TRACH3007995, TRACH3008042, TRACH3008061, TRACH3008093, TRACH3008201, TRACH3008508, TRACH3008535, TRACH3008629, TRACH3008688, TRACH3008697, TRACH3008713, TRACH3008887, TRACH3008902, TRACH3008990, TRACH3009001, TRACH3009008, TRACH3009059, TRACH3009061, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313,

TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016992,

TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758,

TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451,

TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103,

TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037267, TRACH3037288, TRACH3037505, TRACH3037573, TRACH3037696, TRACH3037897, TRACH3038086, TRACH3038399, TSTOM1000135, TSTOM1000186, TSTOM2000235, TSTOM2000442, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000916, TUTER2001286, TUTER2001341, TUTER2001387,

TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN2000046, UMVEN2000069, UMVEN2000121, UMVEN2000133, UMVEN2000152, UMVEN2000354, UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000183, UTERU1000187, UTERU1000192, UTERU1000249, UTERU1000337, UTERU1000339, UTERU2000023, UTERU2000047, UTERU2000074, UTERU2000099, UTERU2000154, UTERU2000197, UTERU2000218, UTERU2000238, UTERU2000243, UTERU2000260,

UTERU2000329, UTERU2000332, UTERU2000349, UTERU2000377, UTERU2000393, UTERU2000418, UTERU2000465, UTERU2000485, UTERU2000517, UTERU2000524, UTERU2000539, UTERU2000541, UTERU2000546, UTERU2000550, UTERU2000569, UTERU2000607, UTERU2000629, UTERU2000649, UTERU2000663, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001747, UTERU2001876, UTERU2002011, UTERU2002176, UTERU2002198, UTERU2002294, UTERU2002332, UTERU2002473, UTERU2002547, UTERU2002662, UTERU2002693, UTERU2002733, UTERU2002736, UTERU2002737, UTERU2002826, UTERU2002841, UTERU2002964, UTERU2002993,

UTERU2003035, UTERU2003057, UTERU2003126, UTERU2003135, UTERU2003321, UTERU2003399, UTERU2003411, UTERU2003456, UTERU2003577, UTERU2003973, UTERU2004015, UTERU2004039, UTERU2004061, UTERU2004073, UTERU2004163, UTERU2004299, UTERU2004461, UTERU2004520, UTERU2004564, UTERU2004664, UTERU2004688, UTERU2004698, UTERU2004861, UTERU2005004, UTERU2005050, UTERU2005069, UTERU2005074, UTERU2005179, UTERU2005292, UTERU2005346, UTERU2005446, UTERU2005449, UTERU2005450, UTERU2005533, UTERU2005593, UTERU2005601, UTERU2005621, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547,

UTERU2006568, UTERU2006643, UTERU2006651, UTERU2006705, UTERU2006899, UTERU2007004, UTERU2007075, UTERU2007128, UTERU2007253, UTERU2007267, UTERU2007444, UTERU2007499, UTERU2007639, UTERU2007724, UTERU2007924, UTERU2007942, UTERU2008018, UTERU2008019, UTERU2008027, UTERU2008077, UTERU2008130, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009510, UTERU2009538, UTERU2009540, UTERU2009776, UTERU2009904, UTERU2009951,

UTERU2009972, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012230, UTERU2012252, UTERU2012333, UTERU2012407, UTERU2012526, UTERU2012581, UTERU2012610, UTERU2012615, UTERU2012688, UTERU2012703, UTERU2012715, UTERU2012741, UTERU2012767, UTERU2012786, UTERU2012890, UTERU2012938, UTERU2012976, UTERU2013048, UTERU2013078,

UTERU2013231, UTERU2013262, UTERU2013280, UTERU2013322, UTERU2013483, UTERU2013491, UTERU2013586, UTERU2013926, UTERU2013976, UTERU2014001, UTERU2014024, UTERU2014223, UTERU2014398, UTERU2014464, UTERU2014548, UTERU2014601, UTERU2014631, UTERU2014668, UTERU2014678, UTERU2014728, UTERU2014998, UTERU2015062, UTERU2015087, UTERU2015108, UTERU2015190, UTERU2015198, UTERU2015202, UTERU2015405, UTERU2015640, UTERU2015653, UTERU2015830, UTERU2015880, UTERU2016147, UTERU2016157, UTERU2016426, UTERU2016464, UTERU2016669, UTERU2016757, UTERU2016761, UTERU2016799, UTERU2016896, UTERU2016902, UTERU2016981, UTERU2017123, UTERU2017303, UTERU2017421, UTERU2017623, UTERU2017632, UTERU2017761, UTERU2017762,

UTERU2017810, UTERU2017988, UTERU2018127, UTERU2018180, UTERU2018200, UTERU2018333, UTERU2018364, UTERU2018514, UTERU2018522, UTERU2018544, UTERU2018566, UTERU2018609, UTERU2018674, UTERU2018712, UTERU2018784, UTERU2018789, UTERU2018811, UTERU2018867, UTERU2018881, UTERU2018955, UTERU2019038, UTERU2019096, UTERU2019163, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023550, UTERU2023651,

UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279,

UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844,

UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422,

UTERU3005460, UTERU3005536, UTERU3005585, UTERU3005767, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010892, UTERU3010919, UTERU3011063, UTERU3011092, UTERU3011273, UTERU3011398, UTERU3011558, UTERU3011579, UTERU3011795, UTERU3011837, UTERU3012293,

UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018711, UTERU3019078

Of the 17,176 genes that include the cDNAs of the present invention, genes whose expression levels were high enough to detect in each of the SALGL, SKMUS, SKNMC, SKNSH, SMINT, SPLEN, and STOMA libraries were analyzed for expression frequency. The analysis results are described below. Each is a relative value, where the expression level over all tissues is taken as 100. The larger this value, the greater the level of expression.

TABLE 37

| Clone Name | SALGL | SKMUS | SKNMC | SKNSH | SMINT | SPLEN | STOMA |
|---|---|---|---|---|---|---|---|
| 3NB691000116 | 0 | 0 | 0 | 0 | 0 | 5.658 | 0 |
| 3NB691000173 | 0 | 0 | 6.155 | 5.422 | 0 | 0 | 0 |
| 3NB692000281 | 0 | 0 | 0 | 1.511 | 0 | 0.766 | 0 |
| 3NB692000330 | 0 | 0 | 3.438 | 0 | 0 | 0.767 | 0 |
| 3NB692000545 | 0 | 0 | 0 | 0 | 0 | 4.908 | 0 |
| 3NB692000912 | 0 | 0 | 0 | 9.678 | 1.658 | 0.817 | 0 |
| 3NB692000973 | 0 | 0 | 0 | 48.489 | 0 | 0 | 0 |
| 3NB692001002 | 0 | 0 | 0 | 26.866 | 0 | 0 | 0 |
| 3NB692001022 | 0 | 0 | 19.398 | 0 | 0 | 0 | 0 |
| 3NB692001334 | 0 | 0 | 0 | 3.686 | 0.947 | 0 | 0 |
| 3NB692001339 | 0 | 16.526 | 0 | 0 | 0 | 0 | 0 |
| 3NB692001442 | 0 | 34.046 | 0 | 0 | 0 | 2.101 | 0 |
| 3NB692001459 | 0 | 0 | 0 | 38.816 | 0 | 0 | 0 |
| 3NB692001528 | 0 | 0 | 18.338 | 0 | 0 | 0 | 0 |
| 3NB692001538 | 0 | 0 | 0 | 0 | 7.010 | 0 | 0 |
| ADRGL1000144 | 0 | 0 | 26.053 | 0 | 0 | 0 | 0 |
| ADRGL2000117 | 0 | 0 | 0 | 0 | 30.118 | 14.848 | 0 |
| ADRGL2000323 | 0 | 0 | 0 | 0 | 0 | 23.091 | 0 |
| ADRGL2002477 | 0 | 0 | 13.262 | 0 | 0 | 2.960 | 0 |
| ASTRO1000165 | 0 | 0 | 0 | 15.219 | 0 | 0 | 0 |
| ASTRO2000480 | 0 | 0 | 22.070 | 0 | 0 | 0 | 0 |
| ASTRO2000914 | 0 | 0 | 15.174 | 0 | 0 | 0 | 0 |
| ASTRO2001076 | 0 | 0 | 0 | 9.759 | 0 | 0 | 0 |
| ASTRO2001249 | 0 | 67.038 | 0 | 0 | 0 | 0 | 0 |
| ASTRO2001806 | 0 | 0 | 0 | 9.823 | 0 | 0 | 0 |
| ASTRO2001823 | 0 | 0 | 0 | 9.967 | 0 | 2.525 | 0 |
| ASTRO2003024 | 0 | 6.678 | 0 | 0 | 3.345 | 0 | 0 |
| ASTRO2005360 | 0 | 0 | 0 | 0 | 0 | 10.378 | 0 |
| ASTRO2005553 | 0 | 0 | 0 | 0 | 0 | 4.508 | 0 |
| ASTRO2005687 | 0 | 0 | 0 | 15.307 | 0 | 0 | 0 |
| ASTRO2005896 | 0 | 0 | 69.231 | 0 | 0 | 0 | 0 |
| ASTRO2007047 | 0 | 0 | 0 | 0 | 0 | 3.636 | 0 |
| ASTRO2010819 | 0 | 0 | 5.245 | 0 | 2.374 | 2.341 | 0 |
| ASTRO2012552 | 0 | 0 | 0 | 10.149 | 0 | 0 | 10.059 |
| ASTRO2013585 | 0 | 0 | 0 | 3.347 | 3.440 | 2.544 | 0 |
| ASTRO2016491 | 0 | 0 | 0 | 0 | 1.777 | 0.876 | 0 |
| ASTRO2018540 | 0 | 0 | 12.123 | 0 | 5.488 | 2.705 | 0 |
| BGGI11000123 | 0 | 0 | 0 | 0 | 0 | 2.438 | 0 |
| BGGI12000054 | 0 | 0 | 0 | 0 | 0 | 0.410 | 0 |
| BGGI12000616 | 0 | 6.300 | 0 | 0 | 0 | 0 | 0 |
| BGGI12000839 | 0 | 0 | 0 | 5.063 | 2.602 | 0 | 0 |
| BGGI12001075 | 0 | 13.948 | 0 | 0 | 0 | 0 | 0 |
| BGGI12001714 | 0 | 0 | 0 | 17.511 | 0 | 0 | 0 |
| BLADE2000256 | 0 | 0 | 0 | 0 | 0 | 19.862 | 0 |
| BLADE2001575 | 0 | 0 | 0 | 0 | 33.455 | 0 | 0 |
| BLADE2004849 | 0 | 0 | 0 | 0 | 0 | 12.894 | 0 |
| BNGH41000137 | 0 | 0 | 0 | 0 | 0 | 7.548 | 0 |
| BNGH41000190 | 0 | 0 | 0 | 0 | 0 | 4.402 | 0 |
| BNGH41000235 | 0 | 0 | 0 | 0 | 22.999 | 0 | 0 |
| BNGH42000360 | 0 | 2.217 | 0 | 0 | 0.555 | 0 | 0 |
| BNGH42000739 | 0 | 0 | 0 | 0 | 2.196 | 0 | 0 |
| BNGH42001576 | 0 | 0 | 0 | 0 | 17.218 | 0 | 0 |
| BNGH42002168 | 0 | 0 | 20.744 | 0 | 0 | 0 | 0 |
| BNGH42007366 | 0 | 0 | 0 | 0 | 0 | 0 | 43.520 |
| BNGH42007709 | 0 | 0 | 0 | 1.580 | 0.812 | 1.201 | 3.133 |
| BNGH42009025 | 0 | 0 | 10.868 | 9.572 | 0 | 0 | 0 |
| BRACE1000042 | 0 | 0 | 0 | 0 | 0 | 5.296 | 0 |
| BRACE1000051 | 0 | 0 | 0 | 0 | 0 | 31.184 | 0 |
| BRACE1000115 | 0 | 0 | 0 | 0 | 0.661 | 0 | 0 |
| BRACE1000131 | 0 | 0 | 0 | 0 | 0 | 5.436 | 0 |
| BRACE2000267 | 0 | 0 | 0 | 0 | 0 | 23.250 | 0 |
| BRACE2000307 | 0 | 0 | 0 | 8.088 | 0 | 0 | 0 |
| BRACE2000332 | 0 | 0 | 0 | 18.638 | 0 | 9.444 | 18.472 |
| BRACE2000347 | 0 | 0 | 0 | 0 | 9.122 | 0 | 0 |
| BRACE2000525 | 0 | 0 | 0 | 0 | 0 | 36.744 | 0 |
| BRACE2000753 | 0 | 0 | 0 | 0 | 0 | 52.985 | 0 |
| BRACE2000864 | 0 | 14.875 | 0 | 0 | 0 | 0 | 3.591 |
| BRACE2001151 | 0 | 0 | 0 | 0 | 0 | 11.229 | 0 |
| BRACE2001313 | 0 | 0 | 0 | 0 | 0 | 52.985 | 0 |
| BRACE2001673 | 0 | 0 | 0 | 0 | 0 | 0 | 1.595 |
| BRACE2001709 | 0 | 0 | 0 | 0 | 11.452 | 16.937 | 0 |
| BRACE2002050 | 0 | 0 | 0 | 19.911 | 0 | 0 | 0 |
| BRACE2002091 | 0 | 0 | 0 | 0 | 0 | 3.674 | 0 |
| BRACE2002441 | 0 | 0 | 8.779 | 0 | 0 | 1.959 | 0 |
| BRACE2002661 | 0 | 0 | 0 | 0 | 10.162 | 5.010 | 19.598 |
| BRACE2002677 | 0 | 0 | 0 | 0 | 0 | 3.358 | 0 |
| BRACE2002807 | 0 | 0 | 0 | 0 | 0 | 56.062 | 0 |
| BRACE2002860 | 0 | 0 | 0 | 0 | 0 | 13.139 | 0 |
| BRACE2003172 | 0 | 0 | 2.533 | 0 | 2.294 | 0 | 0 |
| BRACE2003254 | 0 | 0 | 0 | 0 | 3.195 | 0 | 0 |
| BRACE2003319 | 0 | 0 | 0 | 15.732 | 0 | 0 | 0 |
| BRACE2003827 | 0 | 0 | 0 | 6.016 | 0 | 0 | 0 |
| BRACE2003845 | 0 | 0 | 0 | 0 | 48.233 | 7.926 | 0 |
| BRACE2003846 | 0 | 0 | 0 | 0 | 0 | 1.134 | 0 |
| BRACE2003892 | 0 | 0 | 0 | 0 | 0 | 3.797 | 0 |
| BRACE2003904 | 0 | 0 | 0 | 0 | 0 | 9.020 | 17.642 |
| BRACE2003944 | 0 | 3.648 | 0 | 0 | 0 | 0.901 | 0 |
| BRACE2003954 | 0 | 0 | 0 | 0 | 83.254 | 0 | 0 |
| BRACE2005087 | 0 | 0 | 8.086 | 0 | 0 | 3.609 | 0 |
| BRACE2005298 | 0 | 0 | 0 | 0 | 0 | 52.985 | 0 |
| BRACE2005434 | 0 | 0 | 0 | 9.468 | 0 | 0 | 0 |
| BRACE2006055 | 0 | 0 | 15.594 | 0 | 0 | 0 | 0 |
| BRACE2006162 | 0 | 10.903 | 0 | 0 | 0 | 0 | 0 |
| BRACE2006258 | 0 | 0 | 6.385 | 5.624 | 0 | 0 | 0 |
| BRACE2006264 | 0 | 0 | 0 | 0 | 0 | 14.683 | 0 |
| BRACE2006363 | 0 | 0 | 0 | 0 | 32.705 | 0 | 0 |
| BRACE2006397 | 0 | 0 | 0 | 0 | 0 | 1.027 | 2.009 |
| BRACE2006459 | 0 | 0 | 0 | 0 | 0 | 71.023 | 0 |
| BRACE2006514 | 0 | 0 | 13.545 | 0 | 0 | 0 | 0 |
| BRACE2006537 | 0 | 0 | 0 | 0 | 3.798 | 1.872 | 14.648 |
| BRACE2006598 | 0 | 0 | 0 | 0 | 0 | 50.322 | 0 |
| BRACE2006900 | 0 | 0 | 0 | 3.533 | 0 | 3.580 | 0 |
| BRACE2007064 | 0 | 0 | 0 | 0 | 0 | 2.827 | 0 |
| BRACE2007068 | 0 | 0 | 0 | 0 | 0 | 43.567 | 0 |
| BRACE2007089 | 0 | 0 | 0 | 0 | 0 | 2.946 | 0 |
| BRACE2007109 | 0 | 0 | 0 | 0 | 3.368 | 0 | 0 |
| BRACE2007191 | 0 | 0 | 0 | 4.716 | 0 | 2.390 | 0 |
| BRACE2007254 | 0 | 0 | 0 | 0 | 0 | 12.950 | 0 |
| BRACE2007295 | 0 | 0 | 73.303 | 0 | 0 | 0 | 0 |
| BRACE2007518 | 0 | 0 | 0 | 0 | 0 | 3.948 | 0 |
| BRACE2007567 | 0 | 0 | 0 | 0 | 0 | 7.242 | 9.443 |
| BRACE2007671 | 0 | 0 | 12.052 | 0 | 0 | 0 | 0 |
| BRACE2007708 | 0 | 0 | 0 | 0 | 7.499 | 0 | 0 |
| BRACE2007767 | 0 | 21.937 | 0 | 0 | 0 | 0 | 0 |
| BRACE2008216 | 0 | 0 | 0 | 0 | 46.720 | 0 | 0 |
| BRACE2008358 | 0 | 0 | 0 | 0 | 0 | 7.884 | 0 |
| BRACE200$$385 | 0 | 0 | 0 | 0 | 0 | 3.633 | 0 |
| BRACE2008553 | 0 | 29.305 | 0 | 0 | 0 | 0 | 0 |
| BRACE2008653 | 0 | 0 | 0 | 0 | 0 | 0.917 | 0 |
| BRACE2008968 | 0 | 4.097 | 0 | 0 | 6.155 | 0 | 0 |
| BRACE2008999 | 0 | 49.592 | 0 | 0 | 0 | 0 | 0 |
| BRACE2009037 | 0 | 2.058 | 0 | 2.005 | 1.030 | 0 | 1.987 |
| BRACE2009044 | 0 | 0 | 0 | 0 | 35.405 | 17.454 | 0 |
| BRACE2009366 | 0 | 0 | 0 | 0 | 0 | 6.285 | 0 |
| BRACE2009517 | 0 | 0 | 0 | 0 | 0 | 14.155 | 0 |
| BRACE2009558 | 0 | 0 | 22.414 | 0 | 0 | 5.002 | 19.567 |
| BRACE2011703 | 0 | 0 | 0 | 0 | 0 | 71.023 | 0 |
| BRACE2012061 | 0 | 0 | 0 | 0 | 0 | 29.308 | 0 |
| BRACE2012528 | 0 | 0 | 0 | 0 | 0 | 0.825 | 0 |
| BRACE2012814 | 0 | 0 | 0 | 0 | 5.411 | 0 | 0 |
| BRACE2013911 | 0 | 0 | 0 | 0 | 0 | 13.077 | 0 |
| BRACE2014818 | 0 | 0 | 0 | 0 | 0 | 71.023 | 0 |
| BRACE2015287 | 0 | 0 | 0 | 0 | 8.104 | 0 | 0 |
| BRACE2015969 | 0 | 0 | 0 | 0 | 0 | 71.023 | 0 |
| BRACE2019244 | 0 | 0 | 0 | 0 | 0 | 71.023 | 0 |
| BRACE2019327 | 0 | 63.143 | 0 | 0 | 0 | 0 | 0 |
| BRACE2019371 | 0 | 0 | 0 | 0 | 52.763 | 26.012 | 0 |
| BRACE2021143 | 0 | 0 | 0 | 0 | 27.925 | 0 | 0 |
| BRACE2021478 | 0 | 0 | 0 | 0 | 45.681 | 0 | 0 |
| BRACE2021541 | 0 | 0 | 0 | 0 | 0 | 27.693 | 0 |
| BRACE2022333 | 0 | 0 | 0 | 0 | 0 | 15.444 | 0 |
| BRACE2022389 | 0 | 0 | 0 | 0 | 0 | 0 | 72.312 |
| BRACE2022638 | 0 | 0 | 11.620 | 5.118 | 0 | 2.593 | 0 |
| BRACE2024627 | 0 | 0 | 0 | 0 | 0 | 0 | 83.309 |
| BRACE2026836 | 0 | 48.621 | 0 | 0 | 0 | 0 | 0 |
| BRACE2037295 | 0 | 0 | 25.571 | 0 | 0 | 0 | 0 |
| BRACE2039624 | 0 | 0 | 15.084 | 0 | 3.414 | 1.683 | 6.584 |
| BRACE2043381 | 0 | 0 | 0 | 0 | 0 | 18.723 | 0 |
| BRACE3005938 | 0 | 0 | 0 | 0 | 0 | 5.747 | 0 |
| BRACE3010428 | 0 | 0 | 0 | 0 | 0 | 1.991 | 0 |
| BRALZ2006288 | 0 | 0 | 0 | 0 | 38.482 | 0 | 0 |
| BRALZ2009482 | 0 | 0 | 0 | 0 | 1.311 | 0 | 0 |
| BRAMY2000231 | 0 | 0 | 0 | 0 | 0 | 63.323 | 0 |
| BRAMY2000354 | 0 | 0 | 0 | 0 | 0 | 2.390 | 0 |
| BRAMY2003325 | 0 | 87.489 | 0 | 0 | 0 | 0 | 0 |
| BRAMY2004521 | 0 | 0 | 18.477 | 0 | 0 | 0 | 0 |
| BRAMY2007613 | 0 | 0 | 0 | 0 | 0 | 2.330 | 0 |
| BRAMY2012426 | 0 | 0 | 0 | 0 | 0 | 0 | 42.064 |
| BRAMY2015782 | 0 | 0 | 0 | 0 | 0 | 6.812 | 0 |
| BRAMY2015833 | 0 | 0 | 0 | 0 | 16.267 | 0 | 31.370 |
| BRAMY2019554 | 0 | 0 | 0 | 0 | 8.448 | 0 | 0 |
| BRAMY2020427 | 0 | 0 | 0 | 4.066 | 0 | 0 | 0 |
| BRAMY2021710 | 0 | 0 | 0 | 0 | 26.542 | 13.085 | 0 |
| BRAMY2023719 | 0 | 0 | 0 | 0 | 0 | 10.401 | 0 |
| BRAMY2027739 | 0 | 0 | 0 | 0 | 0 | 2.341 | 0 |
| BRAMY2029988 | 0 | 0 | 0 | 0 | 0 | 20.491 | 0 |
| BRAMY2038832 | 0 | 0 | 0 | 0 | 43.012 | 0 | 0 |
| BRAMY2040095 | 0 | 0 | 0 | 2.347 | 1.206 | 1.189 | 0 |
| BRAMY2045530 | 0 | 0 | 0 | 0 | 0 | 16.412 | 0 |
| BRAMY3004377 | 0 | 0 | 0 | 0 | 0 | 1.443 | 0 |
| BRAMY3010008 | 0 | 0 | 0 | 0 | 0 | 1.012 | 0 |
| BRAMY4002628 | 0 | 0 | 0 | 0 | 0 | 12.356 | 0 |
| BRAWH1000040 | 0 | 0 | 0 | 0 | 4.856 | 2.394 | 0 |
| BRAWH1000083 | 0 | 0 | 0 | 0 | 0 | 7.882 | 0 |
| BRAWH1000093 | 0 | 0 | 0 | 0 | 0 | 6.419 | 0 |
| BRAWH1000094 | 0 | 0 | 0 | 0 | 14.857 | 0 | 0 |
| BRAWH2000177 | 0 | 0 | 0 | 0 | 0 | 13.909 | 0 |
| BRAWH2000417 | 0 | 0 | 0 | 0 | 0 | 1.075 | 0 |
| BRAWH2000460 | 0 | 0 | 0 | 4.191 | 0 | 0 | 0 |
| BRAWH2000522 | 0 | 0 | 0 | 37.336 | 0 | 0 | 0 |
| BRAWH2000588 | 0 | 0 | 0 | 0 | 0 | 2.183 | 0 |
| BRAWH2000633 | 0 | 0 | 0 | 0 | 4.678 | 2.306 | 0 |
| BRAWH2000839 | 0 | 0 | 0 | 0 | 0 | 2.143 | 0 |
| BRAWH2000924 | 0 | 0 | 0 | 0 | 0 | 0.499 | 0 |
| BRAWH2000944 | 0 | 0 | 0 | 0 | 0 | 9.075 | 0 |
| BRAWH2001092 | 0 | 0 | 2.740 | 1.207 | 1.240 | 1.529 | 0 |
| BRAWH2001103 | 0 | 0 | 0 | 0 | 23.189 | 0 | 0 |
| BRAWH2001141 | 0 | 0 | 0 | 0 | 0 | 26.522 | 0 |
| BRAWH2001166 | 0 | 0 | 0 | 0 | 0 | 5.491 | 0 |
| BRAWH2001186 | 0 | 0 | 0 | 0 | 0 | 9.913 | 0 |
| BRAWH2001321 | 0 | 0 | 0 | 0 | 3.029 | 0 | 0 |
| BRAWH2002318 | 0 | 0 | 0 | 0 | 0 | 46.425 | 0 |
| BRAWH2002601 | 0 | 0 | 0 | 0 | 0 | 17.305 | 0 |
| BRAWH2003025 | 0 | 0 | 0 | 0 | 0 | 10.550 | 0 |
| BRAWH2003818 | 0 | 0 | 0 | 20.781 | 0 | 0 | 0 |
| BRAWH2004095 | 0 | 0 | 0 | 0 | 0 | 4.113 | 0 |
| BRAWH2004580 | 0 | 0 | 0 | 0 | 0 | 63.265 | 0 |
| BRAWH2005661 | 0 | 0 | 0 | 0 | 0 | 2.781 | 10.879 |
| BRAWH2006405 | 0 | 0 | 0 | 0 | 45.793 | 0 | 0 |
| BRAWH2006996 | 0 | 0 | 0 | 0 | 0 | 0 | 23.147 |
| BRAWH2007605 | 0 | 0 | 0 | 0 | 0 | 3.568 | 0 |
| BRAWH2007800 | 0 | 0 | 0 | 0 | 0 | 4.086 | 0 |
| BRAWH2008128 | 0 | 0 | 0 | 0 | 77.919 | 0 | 0 |
| BRAWH2008219 | 0 | 0 | 0 | 0 | 0 | 25.068 | 0 |
| BRAWH2008292 | 0 | 0 | 0 | 0 | 0 | 63.499 | 0 |
| BRAWH2009304 | 0 | 0 | 0 | 26.931 | 0 | 0 | 0 |
| BRAWH2011286 | 0 | 0 | 69.568 | 0 | 0 | 0 | 0 |
| BRAWH2011343 | 0 | 0 | 0 | 0 | 0 | 12.204 | 0 |
| BRAWH2014188 | 0 | 0 | 0 | 0 | 0 | 1.245 | 0 |
| BRAWH2014645 | 0 | 0 | 0 | 0 | 3.730 | 3.678 | 0 |
| BRAWH2016106 | 0 | 0 | 0 | 0 | 0 | 63.499 | 0 |
| BRAWH2016166 | 0 | 0 | 0 | 0 | 0 | 13.156 | 0 |
| BRAWH2016209 | 0 | 58.527 | 0 | 0 | 0 | 14.450 | 0 |
| BRAWH2016269 | 0 | 0 | 10.277 | 0 | 0 | 0 | 0 |
| BRAWH2017685 | 0 | 0 | 0 | 0 | 1.791 | 1.766 | 0 |
| BRAWH2018526 | 0 | 0 | 2.399 | 2.113 | 0 | 0 | 0 |
| BRAWH2018527 | 0 | 59.979 | 0 | 0 | 0 | 0 | 0 |
| BRAWH2018729 | 0 | 0 | 0 | 0 | 0 | 5.184 | 0 |
| BRAWH3002467 | 0 | 0 | 0 | 0 | 0 | 63.499 | 0 |
| BRAWH3002853 | 0 | 0 | 0 | 0 | 5.220 | 1.287 | 0 |
| BRAWH3008931 | 0 | 0 | 0 | 0 | 12.222 | 0 | 0 |
| BRAWH3024989 | 0 | 16.693 | 0 | 0 | 0 | 0 | 0 |
| BRCAN1000149 | 0 | 0 | 0 | 0 | 0 | 43.126 | 0 |
| BRCAN2003944 | 0 | 0 | 2.195 | 5.801 | 0 | 0.490 | 0 |
| BRCAN2007409 | 0 | 0 | 0 | 0 | 0 | 10.439 | 0 |
| BRCAN2007700 | 0 | 0 | 0 | 0 | 0 | 63.468 | 0 |
| BRCAN2016619 | 0 | 0 | 0 | 0 | 0 | 45.836 | 0 |
| BRCAN2018566 | 0 | 0 | 0 | 0 | 0 | 43.126 | 0 |
| BRCAN2028036 | 0 | 0 | 0 | 0 | 0 | 4.200 | 0 |
| BRCOC1000040 | 0 | 0 | 0 | 0 | 0 | 5.130 | 0 |
| BRCOC2003187 | 0 | 0 | 0 | 0 | 8.325 | 0 | 10.704 |
| BRCOC2010730 | 0 | 0 | 0 | 0 | 0 | 18.804 | 0 |
| BRCOC2012172 | 0 | 0 | 0 | 0 | 9.926 | 0 | 0 |
| BRCOC2015908 | 0 | 0 | 0 | 0 | 0 | 32.934 | 0 |
| BRCOC2018877 | 0 | 0 | 0 | 0 | 0 | 32.934 | 0 |
| BRHIP1000104 | 0 | 0 | 0 | 0 | 0 | 1.137 | 0 |
| BRHIP2000021 | 0 | 0 | 88.427 | 0 | 0 | 0 | 0 |
| BRHIP2000534 | 0 | 0 | 0 | 0 | 0 | 0.595 | 0 |
| BRHIP2000819 | 0 | 0 | 0 | 0 | 7.623 | 3.758 | 14.701 |
| BRHIP2008269 | 0 | 0 | 0 | 0 | 0 | 48.408 | 0 |
| BRHIP2010217 | 0 | 0 | 43.536 | 0 | 0 | 0 | 0 |
| BRHIP2011491 | 0 | 11.648 | 0 | 0 | 0 | 0 | 0 |
| BRHIP2014063 | 0 | 0 | 0 | 0 | 0 | 63.033 | 0 |
| BRHIP2015153 | 0 | 0 | 0 | 0 | 0 | 17.429 | 0 |
| BRHIP2018712 | 0 | 14.270 | 0 | 0 | 0 | 14.092 | 0 |
| BRHIP2019186 | 0 | 0 | 0 | 0 | 0 | 0 | 13.430 |
| BRHIP2020799 | 0 | 0 | 0 | 0 | 0 | 8.804 | 0 |
| BRHIP2021762 | 0 | 0 | 0 | 0 | 0 | 4.847 | 0 |
| BRHIP2023888 | 0 | 0 | 0 | 0 | 1.298 | 0 | 0 |
| BRHIP2024398 | 0 | 0 | 0 | 0 | 0 | 14.647 | 0 |
| BRHIP2024742 | 0 | 0 | 0 | 0 | 0 | 2.170 | 0 |
| BRHIP3000017 | 0 | 0 | 0 | 0 | 15.559 | 0 | 0 |
| BRHIP3000111 | 0 | 0 | 0 | 0 | 3.613 | 0 | 0 |
| BRHIP3004215 | 0 | 0 | 0 | 0 | 0 | 23.618 | 0 |
| BRSSN2003012 | 0 | 0 | 0 | 0 | 0 | 7.525 | 0 |
| BRSSN2004679 | 0 | 0 | 0 | 0 | 0 | 26.476 | 0 |
| BRSSN2009119 | 0 | 0 | 0 | 0 | 0 | 31.852 | 0 |
| BRSSN2009518 | 0 | 0 | 0 | 11.827 | 6.078 | 0 | 0 |
| BRSSN2011653 | 0 | 0 | 38.627 | 0 | 0 | 0 | 0 |
| BRSSN2014299 | 0 | 0 | 0 | 0 | 0 | 6.428 | 0 |
| BRSSN2015238 | 0 | 0 | 0 | 0 | 0 | 11.911 | 0 |
| BRSTN1000083 | 0 | 0 | 0 | 0 | 0 | 1.111 | 0 |
| BRSTN2011961 | 0 | 0 | 0 | 0 | 1.022 | 1.008 | 0 |
| BRSTN2012069 | 0 | 0.067 | 0.958 | 0.844 | 1.202 | 0.543 | 1.995 |
| BRSTN2012174 | 0 | 0 | 3.153 | 0 | 0.714 | 0 | 0 |
| BRSTN2013171 | 0 | 0 | 0 | 0 | 0 | 6.028 | 0 |
| BRSTN2016470 | 0 | 6.169 | 0 | 0 | 0 | 0 | 1.490 |
| BRSTN2016992 | 0 | 3.468 | 0 | 0 | 0 | 0 | 0 |
| BRSTN2017104 | 0 | 0 | 0 | 0 | 0 | 3.392 | 0 |
| BRTHA2005864 | 0 | 0 | 0 | 0 | 0 | 2.823 | 11.042 |
| BRTHA2016215 | 0 | 0 | 0 | 0 | 0 | 61.070 | 0 |
| BRTHA2026071 | 0 | 0 | 0 | 0 | 43.314 | 0 | 0 |
| BRTHA2027229 | 0 | 0 | 0 | 0 | 3.230 | 0 | 0 |
| BRTHA2033469 | 0 | 0 | 0 | 0 | 0 | 61.070 | 0 |
| BRTHA3003736 | 0 | 0 | 16.769 | 14.770 | 0 | 0 | 0 |
| BRTHA3011194 | 0 | 0 | 27.553 | 0 | 0 | 0 | 0 |
| BRTHA3021971 | 0 | 0 | 0 | 0 | 6.849 | 0 | 0 |
| CHONS2002829 | 0 | 0 | 0 | 0 | 0 | 2.842 | 11.118 |
| COLON2002443 | 0 | 0 | 0 | 0 | 0 | 0 | 30.687 |
| CTONG1000010 | 0 | 0 | 0 | 0 | 0 | 11.278 | 0 |
| CTONG1000052 | 0 | 0 | 0 | 0 | 0 | 3.153 | 0 |
| CTONG1000094 | 0 | 0 | 0 | 0 | 0 | 0.678 | 0 |
| CTONG1000241 | 0 | 0 | 0 | 0 | 0 | 3.690 | 0 |
| CTONG1000341 | 0 | 0 | 0 | 0 | 0 | 48.294 | 0 |
| CTONG2000063 | 0 | 0 | 16.688 | 0 | 0 | 0 | 0 |
| CTONG2000452 | 0 | 0 | 0 | 0 | 0 | 0 | 19.820 |
| CTONG2000508 | 0 | 0 | 0 | 0 | 0 | 2.748 | 0 |
| CTONG2001226 | 0 | 0 | 0 | 0 | 0 | 0 | 51.655 |
| CTONG2001932 | 0 | 0 | 0 | 0 | 0 | 17.973 | 0 |
| CTONG2002270 | 0 | 0 | 0 | 0 | 0 | 0.923 | 0 |
| CTONG2002558 | 0 | 0 | 0 | 0 | 0 | 48.294 | 0 |
| CTONG2002744 | 0 | 0 | 0 | 0 | 0 | 1.024 | 0 |
| CTONG2003298 | 0 | 0 | 0 | 0 | 0 | 1.262 | 0 |
| CTONG2003361 | 0 | 0 | 0 | 0 | 0 | 40.876 | 0 |
| CTONG2003680 | 0 | 0 | 80.715 | 0 | 0 | 0 | 0 |
| CTONG2004397 | 0 | 0 | 0 | 0 | 0 | 48.294 | 0 |
| CTONG2005553 | 0 | 0 | 0 | 0 | 0 | 1.246 | 0 |
| CTONG2006004 | 0 | 0 | 0 | 0 | 0 | 3.672 | 0 |
| CTONG2006562 | 0 | 0 | 10.914 | 0 | 0 | 0 | 0 |
| CTONG2006611 | 0 | 0 | 0 | 0 | 0 | 12.562 | 0 |
| CTONG2006709 | 0 | 0 | 0 | 0 | 0 | 8.998 | 0 |
| CTONG2006932 | 0 | 0 | 0 | 0 | 0 | 1.914 | 0 |
| CTONG2007007 | 0 | 13.119 | 0 | 0 | 0 | 0 | 0 |
| CTONG2007078 | 0 | 0 | 9.962 | 8.775 | 0 | 0 | 0 |
| CTONG2007500 | 0 | 0 | 0 | 0 | 5.308 | 2.617 | 0 |
| CTONG2008506 | 0 | 0 | 0 | 6.461 | 0 | 0 | 0 |
| CTONG2010348 | 0 | 0 | 0 | 0 | 0 | 3.080 | 0 |
| CTONG2010408 | 0 | 0 | 0 | 0 | 30.235 | 0 | 0 |
| CTONG2011716 | 0 | 0 | 0 | 0 | 0 | 48.294 | 0 |
| CTONG2012425 | 0 | 0 | 0 | 0 | 0 | 48.294 | 0 |
| CTONG2012452 | 0 | 0 | 2.776 | 0 | 2.513 | 0 | 4.847 |
| CTONG2012847 | 0 | 0 | 0 | 0 | 0 | 0 | 11.719 |
| CTONG2013348 | 0 | 0 | 0 | 0 | 0 | 9.570 | 0 |
| CTONG2013907 | 0 | 0 | 16.806 | 0 | 0 | 3.750 | 0 |
| CTONG2013985 | 0 | 0 | 45.651 | 0 | 0 | 0 | 0 |
| CTONG2014165 | 0 | 0 | 0 | 0 | 0 | 16.752 | 0 |
| CTONG2014389 | 0 | 0 | 0 | 0 | 9.321 | 0 | 0 |
| CTONG2014954 | 0 | 0 | 0 | 0 | 43.143 | 0 | 0 |
| CTONG2015091 | 0 | 0 | 6.331 | 0 | 0 | 0 | 0 |
| CTONG2015815 | 0 | 0 | 68.575 | 0 | 0 | 0 | 0 |
| CTONG2016185 | 0 | 0 | 0 | 0 | 0 | 4.522 | 0 |
| CTONG2016575 | 0 | 0 | 3.876 | 0 | 0 | 0.865 | 0 |
| CTONG2016869 | 0 | 0 | 0 | 0 | 0 | 4.595 | 0 |
| CTONG2017444 | 0 | 0 | 0 | 0 | 0 | 0 | 25.060 |
| CTONG2017998 | 0 | 0 | 0 | 0 | 0 | 4.358 | 0 |
| CTONG2018211 | 0 | 0 | 0 | 0 | 12.035 | 0 | 0 |
| CTONG2018383 | 0 | 0 | 0 | 0 | 0 | 7.864 | 0 |
| CTONG2018637 | 0 | 0 | 0 | 0 | 0 | 11.413 | 0 |
| CTONG2018898 | 0 | 0 | 0 | 0 | 0 | 5.027 | 0 |
| CTONG2021289 | 0 | 0 | 0 | 0 | 0 | 17.899 | 0 |
| CTONG2028124 | 0 | 0 | 0 | 0 | 3.547 | 0.874 | 0 |
| CTONG3003972 | 0 | 0 | 0 | 0 | 0 | 48.294 | 0 |
| CTONG3008894 | 0 | 0 | 5.667 | 0 | 0 | 0 | 0 |
| CTONG3009239 | 0 | 0 | 0 | 0 | 0 | 2.512 | 0 |
| D3OST1000270 | 0 | 18.206 | 0 | 0 | 0 | 0 | 0 |
| D3OST2002068 | 0 | 0 | 1.675 | 0 | 0 | 0.374 | 0 |
| D90ST2002608 | 0 | 0 | 0 | 0 | 0 | 19.379 | 0 |
| DFNES1000083 | 0 | 0 | 17.535 | 0 | 0 | 0 | 0 |
| DFNES1000150 | 0 | 0 | 0 | 0 | 0 | 1.943 | 0 |
| DFNES2000153 | 0 | 0 | 0 | 21.984 | 0 | 0 | 0 |
| DFNES2000443 | 0 | 0 | 5.292 | 0 | 0 | 0 | 0 |
| DFNES2003081 | 0 | 0 | 0 | 0 | 0 | 0 | 53.591 |
| DFNES2005766 | 0 | 0 | 0 | 0 | 0 | 3.189 | 0 |
| DFNES2006346 | 0 | 0 | 0 | 0 | 0 | 12.289 | 0 |
| DFNES2007634 | 0 | 0 | 0 | 0 | 0 | 1.546 | 0 |
| FCBBF1000027 | 0 | 0 | 2.855 | 0 | 0 | 1.274 | 0 |
| FCBBF1000077 | 0 | 0 | 0 | 0 | 0 | 1.409 | 0 |
| FCBBF1000197 | 0 | 0 | 0 | 0 | 0 | 1.858 | 0 |
| FCBBF1000294 | 0 | 0 | 0 | 0 | 0 | 1.873 | 0 |
| FCBBF1000367 | 0 | 0 | 11.842 | 0 | 0 | 0 | 0 |
| FCBBF1000403 | 0 | 0 | 0 | 0 | 29.596 | 0 | 0 |
| FCBBF1000412 | 0 | 0 | 0 | 0 | 6.829 | 0 | 0 |
| FCBBF1000437 | 0 | 0 | 0 | 0 | 0 | 44.494 | 0 |
| FCBBF1000466 | 0 | 0 | 4.964 | 0 | 2.247 | 0 | 0 |
| FCBBF1000574 | 0 | 0 | 0 | 0 | 7.325 | 7.222 | 0 |
| FCBBF1000732 | 0 | 39.461 | 0 | 0 | 0 | 4.871 | 0 |
| FCBBF1000760 | 0 | 0 | 0 | 0 | 0 | 3.418 | 0 |
| FCBBF2000591 | 0 | 0 | 0 | 0 | 0 | 0.499 | 0 |
| FCBBF2001211 | 0 | 0 | 0 | 0 | 22.614 | 0 | 0 |
| FCBBF2001291 | 0 | 0 | 0 | 0 | 0 | 6.619 | 0 |
| FCBBF2002801 | 0 | 0 | 0 | 17.821 | 0 | 0 | 0 |
| FCBBF2002928 | 0 | 0 | 24.255 | 0 | 0 | 0 | 0 |
| FCBBF2003549 | 0 | 0 | 0 | 78.798 | 0 | 0 | 0 |
| FCBBF3000110 | 0 | 0 | 0 | 0 | 0 | 11.235 | 0 |
| FCBBF3000115 | 0 | 0 | 0 | 0 | 0 | 6.943 | 0 |
| FCBBF3000227 | 0 | 79.227 | 0 | 0 | 0 | 0 | 0 |
| FCBBF3000388 | 0 | 0 | 0 | 0 | 8.716 | 4.297 | 0 |
| FCBBF3001470 | 0 | 0 | 0 | 0 | 0 | 2.361 | 0 |
| FCBBF3003800 | 0 | 0 | 0 | 0 | 1.390 | 1.371 | 0 |
| FCBBF3004473 | 0 | 0 | 0 | 0 | 0 | 1.999 | 0 |
| FCBBF3005320 | 0 | 0 | 0 | 0 | 0 | 4.746 | 0 |
| FCBBF3005330 | 0 | 0 | 0 | 0 | 2.445 | 0 | 0 |
| FCBBF3005497 | 0 | 0 | 0 | 10.796 | 0 | 1.368 | 0 |
| FCBBF3008153 | 0 | 0 | 0 | 0 | 0 | 9.518 | 0 |
| FCBBF3008938 | 0 | 0 | 0 | 0 | 0 | 48.497 | 0 |
| FCBBF3009479 | 0 | 0 | 0 | 0 | 34.906 | 0 | 0 |
| FCBBF3009541 | 0 | 0 | 12.854 | 0 | 0 | 0 | 0 |
| FCBBF3013589 | 0 | 0 | 64.676 | 0 | 0 | 0 | 0 |
| FCBBF3014260 | 0 | 0 | 17.601 | 0 | 0 | 0 | 0 |
| FCBBF3016928 | 0 | 0 | 0 | 0 | 0 | 1.524 | 0 |
| FCBBF3018796 | 0 | 21.875 | 0 | 0 | 0 | 0 | 0 |
| FCBBF3019714 | 0 | 0 | 0 | 0 | 7.228 | 0 | 0 |
| FCBBF3020599 | 0 | 0 | 0 | 0 | 0 | 48.497 | 0 |
| FCBBF3023667 | 0 | 0 | 1.035 | 2.735 | 0.469 | 0.462 | 0 |
| FCBBF3026308 | 0 | 0 | 0 | 0 | 0 | 22.234 | 0 |
| FCBBF3028593 | 0 | 0 | 0 | 0 | 14.248 | 0 | 0 |
| FCBBF4000446 | 0 | 0 | 0 | 0 | 0 | 0 | 5.112 |
| FCBBF4000500 | 0 | 61.755 | 0 | 0 | 0 | 0 | 0 |
| FEBRA1000022 | 0 | 0 | 41.028 | 0 | 0 | 0 | 0 |
| FEBRA1000190 | 0 | 0 | 61.954 | 0 | 0 | 0 | 0 |
| FEBRA2000105 | 0 | 0 | 0 | 0 | 12.654 | 0 | 0 |
| FEBRA2000129 | 0 | 0 | 0 | 0 | 18.347 | 0 | 0 |
| FEBRA2000210 | 0 | 0 | 0 | 0 | 0 | 1.230 | 0 |
| FEBRA2000228 | 0 | 0 | 0 | 0 | 13.939 | 0 | 0 |
| FEBRA2000452 | 0 | 0 | 0 | 0 | 0 | 1.745 | 0 |
| FEBRA2000510 | 0 | 0 | 0 | 0 | 0 | 6.419 | 0 |
| FEBRA2000733 | 0 | 0 | 1.314 | 3.471 | 1.189 | 0.293 | 2.294 |
| FEBRA2000740 | 0 | 7.969 | 0 | 0 | 0 | 0 | 0 |
| FEBRA2000762 | 0 | 0 | 0 | 0 | 21.951 | 0 | 0 |
| FEBRA2000787 | 0 | 0 | 0 | 0 | 6.970 | 3.436 | 0 |
| FEBRA2000880 | 0 | 0 | 0 | 0 | 0 | 18.435 | 0 |
| FEBRA2001294 | 0 | 0 | 0 | 0 | 0 | 4.204 | 0 |
| FEBRA2001492 | 0 | 0 | 0 | 0 | 0 | 5.630 | 0 |
| FEBRA2001523 | 0 | 0 | 0 | 0 | 0 | 14.791 | 0 |
| FEBRA2001571 | 0 | 0 | 0 | 0 | 0 | 10.945 | 0 |
| FEBRA2001581 | 0 | 0 | 0 | 0 | 0 | 21.399 | 0 |
| FEBRA2001669 | 0 | 0 | 0 | 0 | 0 | 1.864 | 0 |
| FEBRA2001828 | 0 | 0 | 0 | 0 | 0 | 9.465 | 0 |
| FEBRA2002352 | 0 | 0 | 0 | 0 | 0 | 6.039 | 0 |
| FEBRA2002933 | 0 | 0 | 0 | 0 | 0 | 1.288 | 0 |
| FEBRA2003100 | 0 | 0 | 0 | 0 | 2.324 | 0 | 0 |
| FEBRA2003258 | 0 | 0 | 0 | 38.852 | 0 | 0 | 0 |
| FEBRA2003436 | 0 | 0 | 0 | 0 | 19.617 | 0 | 0 |
| FEBRA2003750 | 0 | 0 | 0 | 0 | 0 | 0 | 12.423 |
| FEBRA2003926 | 0 | 0 | 0 | 0 | 2.463 | 0 | 0 |
| FEBRA2003930 | 0 | 0 | 0 | 0 | 6.342 | 3.127 | 0 |
| FEBRA2004042 | 0 | 0 | 0 | 0 | 0 | 3.690 | 0 |
| FEBRA2004224 | 0 | 0 | 0 | 7.949 | 0 | 0 | 0 |
| FEBRA2004293 | 0 | 0 | 0 | 0 | 0 | 9.249 | 0 |
| FEBRA2004412 | 0 | 0 | 0 | 20.159 | 0 | 0 | 0 |
| FEBRA2004628 | 0 | 0 | 0 | 0 | 0 | 14.887 | 0 |
| FEBRA2005572 | 0 | 0 | 0 | 0 | 0 | 6.199 | 0 |
| FEBRA2005734 | 0 | 0 | 0 | 0 | 5.959 | 0 | 0 |
| FEBRA2005778 | 0 | 0 | 0 | 41.192 | 0 | 0 | 0 |
| FEBRA2005788 | 0 | 34.776 | 0 | 0 | 0 | 0 | 0 |
| FEBRA2005824 | 0 | 0 | 0 | 0 | 1.316 | 0.649 | 0 |
| FEBRA2006092 | 0 | 0 | 0 | 4.269 | 2.194 | 0 | 0 |
| FEBRA2006270 | 0 | 0 | 9.113 | 0 | 0 | 0 | 0 |
| FEBRA2006346 | 0 | 0 | 0 | 0 | 52.422 | 0 | 0 |
| FEBRA2006401 | 0 | 0 | 0 | 0 | 0 | 0.437 | 0 |
| FEBRA2006491 | 0 | 0 | 0 | 0 | 0 | 25.833 | 0 |
| FEBRA2006736 | 0 | 0 | 0 | 0 | 52.422 | 0 | 0 |
| FEBRA2007622 | 0 | 0 | 8.042 | 0 | 0 | 0 | 0 |
| FEBRA2007801 | 0 | 0 | 0 | 0 | 0 | 8.589 | 0 |
| FEBRA2007901 | 0 | 0 | 0 | 0 | 0 | 6.447 | 0 |
| FEBRA2008081 | 0 | 0 | 0 | 0 | 0 | 3.696 | 0 |
| FEBRA2008087 | 0 | 0 | 1.397 | 1.231 | 1.265 | 0.312 | 0 |
| FEBRA2008201 | 0 | 0 | 0 | 16.466 | 0 | 0 | 0 |
| FEBRA2008210 | 0 | 0 | 0 | 0 | 23.668 | 0 | 0 |
| FEBRA2008302 | 0 | 0 | 0 | 0 | 0 | 3.703 | 0 |
| FEBRA2008341 | 0 | 0 | 0 | 0 | 23.180 | 11.427 | 0 |
| FEBRA2008475 | 0 | 0 | 0 | 57.933 | 0 | 0 | 0 |
| FEBRA2008583 | 0 | 0 | 0 | 0 | 0.466 | 0 | 0 |
| FEBRA2008755 | 0 | 0 | 0 | 0 | 0 | 2.858 | 0 |
| FEBRA2009022 | 0 | 0 | 26.033 | 0 | 0 | 5.810 | 0 |
| FEBRA2009541 | 0 | 0 | 0 | 0 | 0 | 28.935 | 0 |
| FEBRA2014417 | 0 | 0 | 0 | 3.707 | 0 | 0 | 0 |
| FEBRA2018476 | 0 | 0 | 0 | 7.502 | 0 | 1.901 | 7.436 |
| FEBRA2019711 | 0 | 0 | 16.484 | 0 | 0 | 3.679 | 0 |
| FEBRA2020977 | 0 | 0 | 0 | 0 | 0 | 0 | 35.859 |
| FEBRA2021636 | 0 | 0 | 0 | 0 | 0 | 35.199 | 0 |
| FEBRA2021966 | 0 | 0 | 0 | 29.618 | 0 | 7.504 | 0 |
| FEBRA2022055 | 0 | 0 | 0 | 0 | 0 | 3.703 | 3.621 |
| FEBRA2023227 | 0 | 0 | 39.925 | 0 | 0 | 0 | 0 |
| FEBRA2025427 | 0 | 0 | 0 | 0 | 8.747 | 0 | 0 |
| FEHRT2000364 | 0 | 0 | 0 | 0 | 0 | 7.710 | 0 |
| FELIV1000153 | 0 | 0 | 1.286 | 1.132 | 0 | 1.148 | 1.122 |
| HCASM2000363 | 0 | 0 | 0 | 0 | 0 | 1.183 | 0 |
| HCHON1000015 | 0 | 54.915 | 0 | 0 | 0 | 2.712 | 0 |
| HCHON1000030 | 0 | 0 | 4.242 | 1.245 | 1.920 | 1.893 | 0 |
| HCHON1000131 | 0 | 0 | 0 | 0 | 0 | 14.275 | 0 |
| HCHON1000166 | 0 | 0 | 0 | 0 | 21.230 | 0 | 0 |
| HCHON2000028 | 0 | 0 | 0 | 0 | 0 | 2.760 | 0 |
| HCHON2000056 | 0 | 0 | 0 | 0 | 1.001 | 1.480 | 0 |
| HCHON2000087 | 0 | 0 | 0 | 0 | 0 | 2.152 | 0 |
| HCHON2000160 | 0 | 0 | 15.694 | 0 | 0 | 3.502 | 0 |
| HCHON2000226 | 0 | 0 | 0 | 0 | 0 | 2.806 | 0 |
| HCHON2000244 | 0 | 0 | 0 | 0 | 4.643 | 0 | 0 |
| HCHON2000295 | 0 | 3.086 | 0 | 0 | 0 | 0.762 | 0 |
| HCHON2000323 | 0 | 0 | 0 | 0 | 0 | 3.223 | 0 |
| HCHON2000373 | 0 | 0 | 0 | 0 | 0 | 0 | 6.897 |
| HCHON2000475 | 0 | 0 | 10.376 | 0 | 0 | 0 | 0 |
| HCHON2000626 | 0 | 0 | 0 | 0 | 4.148 | 0 | 0 |
| HCHON2000738 | 0 | 0 | 23.211 | 0 | 0 | 0 | 0 |
| HCHON2000751 | 0 | 0 | 0 | 0 | 0 | 6.211 | 0 |
| HCHON2000826 | 0 | 0 | 0 | 0 | 0 | 0 | 4.870 |
| HCHON2001039 | 0 | 0 | 0 | 0 | 3.937 | 0 | 0 |
| HCHON2001200 | 0 | 0 | 3.372 | 0 | 0 | 0 | 0 |
| HCHON2001217 | 0 | 0 | 0 | 7.147 | 0 | 0 | 0 |
| HCHON2001359 | 0 | 0 | 0 | 0 | 3.004 | 0 | 0 |
| HCHON2001407 | 0 | 0 | 6.156 | 0 | 0 | 0 | 0 |
| HCHON2001505 | 0 | 0 | 0 | 0 | 0 | 7.817 | 0 |
| HCHON2003642 | 0 | 0 | 0 | 0 | 0 | 21.564 | 0 |
| HCHON2004002 | 0 | 0 | 0 | 0 | 2.770 | 5.463 | 5.343 |
| HCHON2006250 | 0 | 0 | 0 | 0 | 0 | 2.722 | 0 |
| HCHON2007650 | 0 | 0 | 0 | 0 | 0 | 10.594 | 0 |
| HEART1000010 | 0 | 0 | 0 | 0 | 0 | 5.943 | 23.250 |
| HEART1000074 | 0 | 0 | 5.918 | 0 | 8.036 | 2.641 | 0 |
| HEART1000132 | 0 | 20.465 | 0 | 0 | 0 | 0 | 0 |
| HEART1000149 | 0 | 0 | 0 | 0 | 6.628 | 0 | 0 |
| HEART1000185 | 0 | 0 | 0 | 0 | 8.369 | 0 | 0 |
| HEART2000444 | 0 | 0 | 0 | 0 | 29.718 | 0 | 0 |
| HEART2000506 | 0 | 0 | 0 | 0 | 0 | 3.051 | 11.934 |
| HEART2000520 | 0 | 40.642 | 0 | 0 | 0 | 0 | 0 |
| HEART2003836 | 0 | 43.489 | 0 | 0 | 0 | 0 | 0 |
| HEART2003852 | 0 | 27.453 | 0 | 0 | 0 | 0 | 0 |
| HEART2006195 | 0 | 0 | 0 | 0 | 0 | 16.487 | 0 |
| HEART2006521 | 0 | 16.853 | 0 | 0 | 0 | 0 | 0 |
| HHDPC1000083 | 0 | 0 | 0 | 6.721 | 3.454 | 0 | 6.661 |
| HHDPC1000163 | 0 | 0 | 0 | 0 | 4.438 | 2.188 | 0 |
| HHDPC2000055 | 0 | 0 | 0 | 0 | 0 | 3.181 | 0 |
| HHDPC2000115 | 0 | 0 | 0 | 17.962 | 0 | 4.551 | 0 |
| HHDPC2000149 | 0 | 0 | 0 | 0 | 15.767 | 0 | 0 |
| HHDPC2003439 | 0 | 0 | 0 | 1.746 | 0.897 | 0.885 | 0 |
| HHDPC2003983 | 0 | 5.886 | 2.171 | 1.912 | 0 | 1.938 | 0 |
| HHDPC2005794 | 0 | 0 | 0 | 0 | 0 | 13.838 | 0 |
| HHDPC2006460 | 0 | 0 | 0 | 0 | 0 | 33.071 | 0 |
| HHDPC2007267 | 0 | 0 | 0 | 0 | 0 | 0.849 | 0 |
| HLUNG1000037 | 0 | 0 | 0 | 0 | 31.007 | 0 | 0 |
| HLUNG1000055 | 0 | 0 | 0 | 0 | 0 | 1.883 | 0 |
| HLUNG1000064 | 0 | 0 | 0 | 0 | 0 | 11.846 | 0 |
| HLUNG2000063 | 0 | 0 | 0 | 0 | 17.406 | 8.581 | 0 |
| HLUNG2000412 | 0 | 0 | 0 | 0 | 9.449 | 0 | 18.222 |
| HLUNG2000501 | 0 | 0 | 0 | 0 | 0 | 1.492 | 5.838 |
| HLUNG2000950 | 0 | 0 | 0 | 0 | 0 | 21.507 | 0 |
| HLUNG2001439 | 0 | 0 | 6.875 | 0 | 0 | 0 | 0 |
| HLUNG2001518 | 0 | 0 | 0 | 0 | 0 | 7.609 | 0 |
| HLUNG2002145 | 0 | 0 | 0 | 0 | 49.034 | 0 | 0 |
| HLUNG2002334 | 0 | 0 | 0 | 0 | 0 | 15.991 | 0 |
| HLUNG2002465 | 0 | 0 | 0 | 0 | 0 | 10.520 | 8.231 |
| HLUNG2002648 | 0 | 0 | 0 | 0 | 12.754 | 0 | 0 |
| HLUNG2002949 | 0 | 0 | 0 | 0 | 0 | 32.171 | 0 |
| HLUNG2003003 | 0 | 0 | 0 | 0 | 0 | 13.902 | 0 |
| HLUNG2003042 | 0 | 0 | 0 | 0 | 8.400 | 0 | 0 |
| HLUNG2003331 | 0 | 0 | 0 | 0 | 0 | 14.402 | 0 |
| HLUNG2003778 | 0 | 0 | 0 | 0 | 0 | 8.974 | 0 |
| HLUNG2004159 | 0 | 0 | 0 | 0 | 0 | 6.308 | 0 |
| HLUNG2004388 | 0 | 0 | 34.807 | 0 | 0 | 7.768 | 0 |
| HLUNG2005230 | 0 | 0 | 0 | 0 | 8.066 | 11.929 | 0 |
| HLUNG2006812 | 0 | 0 | 0 | 0 | 0 | 5.144 | 0 |
| HLUNG2006843 | 0 | 0 | 0 | 0 | 0 | 18.056 | 0 |
| HLUNG2007163 | 0 | 0 | 0 | 0 | 0 | 23.437 | 0 |
| HLUNG2008225 | 0 | 0 | 0 | 0 | 0 | 28.702 | 0 |
| HLUNG2008637 | 0 | 0 | 0 | 0 | 5.961 | 0 | 0 |
| HLUNG2008885 | 0 | 0 | 0 | 0 | 0 | 18.779 | 0 |
| HLUNG2009225 | 0 | 0 | 0 | 0 | 0 | 5.247 | 0 |
| HSYRA1000062 | 0 | 0 | 0 | 2.941 | 0 | 0 | 0 |
| HSYRA1000119 | 0 | 0 | 0 | 3.157 | 3.245 | 1.600 | 0 |
| HSYRA1000137 | 0 | 0 | 0 | 0 | 0 | 6.483 | 0 |
| HSYRA1000178 | 0 | 0 | 0 | 0 | 8.848 | 0 | 0 |
| HSYRA2000159 | 0 | 0 | 0 | 0 | 0.425 | 0 | 0 |
| HSYRA2000190 | 0 | 16.735 | 0 | 0 | 0 | 0 | 0 |
| HSYRA2000224 | 0 | 0 | 0 | 0 | 0 | 0 | 12.237 |
| HSYRA2000232 | 0 | 0 | 0 | 5.077 | 0 | 0 | 0 |
| HSYRA2000248 | 0 | 0 | 0 | 4.667 | 2.398 | 0 | 0 |
| HSYRA2000255 | 0 | 0 | 0 | 0 | 1.583 | 0 | 0 |
| HSYRA2000332 | 0 | 0 | 0 | 0 | 0 | 2.714 | 0 |
| HSYRA2000640 | 0 | 0 | 0 | 0 | 0 | 1.482 | 17.392 |
| HSYRA2000743 | 0 | 0 | 0 | 0 | 0 | 2.693 | 0 |
| HSYRA2000925 | 0 | 0 | 0 | 0 | 0.782 | 0 | 0 |
| HSYRA2001103 | 0 | 0 | 0 | 0 | 5.986 | 0 | 0 |
| HSYRA2001138 | 0 | 1.796 | 0 | 0 | 1.799 | 1.330 | 0 |
| HSYRA2001225 | 0 | 0 | 5.763 | 0 | 0 | 0 | 0 |
| HSYRA2001396 | 0 | 0 | 0 | 0 | 0 | 2.643 | 0 |
| HSYRA2001420 | 0 | 0 | 0 | 0 | 19.661 | 0 | 0 |
| HSYRA2001476 | 0 | 0 | 0 | 0 | 1.704 | 0 | 0 |
| HSYRA2001567 | 0 | 0 | 0 | 0 | 0 | 11.696 | 0 |
| HSYRA2001574 | 0 | 0 | 5.865 | 0 | 0 | 0 | 0 |
| HSYRA2001595 | 0 | 0 | 4.441 | 0 | 0 | 0 | 0 |
| HSYRA2001983 | 0 | 0 | 0 | 0 | 17.031 | 0 | 0 |
| HSYRA2007650 | 0 | 0 | 0 | 0 | 0 | 8.275 | 0 |
| IMR321000158 | 0 | 0 | 0 | 41.812 | 0 | 1.324 | 5.180 |
| IMR321000210 | 0 | 0 | 0 | 0 | 3.577 | 0 | 0 |
| IMR321000219 | 0 | 0 | 4.376 | 1.927 | 0 | 0 | 0 |
| IMR321000242 | 0 | 0 | 0 | 0 | 0 | 9.783 | 0 |
| IMR321000253 | 0 | 0 | 7.940 | 0 | 3.594 | 1.772 | 6.931 |
| IMR322000072 | 0 | 0 | 0 | 0 | 5.663 | 0 | 0 |
| IMR322000121 | 0 | 0 | 0 | 0 | 0 | 1.939 | 0 |
| IMR322000127 | 0 | 0 | 0 | 0 | 5.466 | 5.390 | 0 |
| IMR322000144 | 0 | 11.493 | 0 | 0 | 0 | 0 | 0 |
| IMR322000152 | 0 | 0 | 63.150 | 0 | 0 | 0 | 0 |
| IMR322000178 | 0 | 0 | 0 | 0 | 6.563 | 0 | 0 |
| IMR322000223 | 0 | 0 | 0 | 4.675 | 0 | 2.369 | 0 |
| IMR322000243 | 0 | 0 | 0 | 0 | 0 | 1.493 | 5.838 |
| IMR322000302 | 0 | 0 | 0 | 40.734 | 0 | 0 | 0 |
| IMR322000316 | 0 | 0 | 0 | 2.849 | 0 | 0 | 0 |
| IMR322000374 | 0 | 0 | 0 | 37.822 | 0 | 0 | 0 |
| IMR322000730 | 0 | 0.966 | 0 | 0.941 | 0 | 0.238 | 0.933 |
| IMR322000742 | 0 | 0 | 0 | 0 | 5.843 | 0 | 0 |
| IMR322000935 | 0 | 10.549 | 0 | 0 | 0 | 0 | 0 |
| IMR322000973 | 0 | 0 | 0 | 0 | 2.845 | 0 | 0 |
| IMR322001049 | 0 | 0 | 0 | 0 | 0 | 10.077 | 0 |
| IMR322001218 | 0 | 7.888 | 0 | 0 | 0 | 0 | 0 |
| IMR322001317 | 0 | 38.949 | 0 | 0 | 0 | 0 | 0 |
| IMR322001435 | 0 | 0 | 0 | 0 | 0 | 4.903 | 6.393 |
| IMR322001438 | 0 | 0 | 64.780 | 0 | 0 | 0 | 0 |
| IMR322001541 | 0 | 23.840 | 0 | 0 | 0 | 0 | 0 |
| IMR322001584 | 0 | 0 | 0 | 0 | 0 | 4.343 | 0 |
| IMR322001600 | 0 | 0 | 3.766 | 0 | 0 | 0 | 0 |
| JCMLC2000273 | 0 | 0 | 0 | 0 | 0 | 5.376 | 0 |
| KIDNE1000145 | 0 | 0 | 0 | 0 | 0 | 9.622 | 0 |
| KIDNE2000085 | 0 | 0 | 0 | 0 | 0 | 3.578 | 0 |
| KIDNE2000252 | 0 | 0 | 0 | 5.868 | 0 | 0 | 0 |
| KIDNE2000266 | 0 | 0 | 0 | 0 | 14.858 | 7.325 | 0 |
| KIDNE2000383 | 0 | 0 | 0 | 0 | 0 | 8.567 | 0 |
| KIDNE2000497 | 0 | 22.095 | 0 | 0 | 0 | 0 | 0 |
| KIDNE2000574 | 0 | 0 | 5.835 | 0 | 0 | 1.302 | 0 |
| KIDNE2000777 | 0 | 0 | 0 | 0 | 13.468 | 0 | 12.986 |
| KIDNE2000833 | 0 | 0 | 0 | 49.464 | 25.421 | 0 | 0 |
| KIDNE2000909 | 0 | 0 | 0 | 0 | 14.489 | 3.571 | 0 |
| KIDNE2001162 | 0 | 0 | 0 | 16.024 | 4.941 | 1.624 | 0 |
| KIDNE2001373 | 0 | 0 | 0 | 0 | 25.141 | 0 | 0 |
| KIDNE2001467 | 0 | 0 | 0 | 0 | 0 | 0.453 | 0 |
| KIDNE2002252 | 0 | 0 | 0 | 0 | 0 | 3.792 | 0 |
| KIDNE2002872 | 0 | 0 | 0 | 0 | 0 | 0.995 | 0 |
| KIDNE2002883 | 0 | 0 | 0 | 0 | 0 | 3.946 | 0 |
| KIDNE2003305 | 0 | 0 | 0 | 0 | 2.970 | 0 | 0 |
| KIDNE2003507 | 0 | 0 | 0 | 0 | 0 | 5.890 | 0 |
| KIDNE2003941 | 0 | 0 | 3.932 | 0 | 0 | 0 | 0 |
| KIDNE2003994 | 0 | 0 | 62.857 | 0 | 0 | 0 | 0 |
| KIDNE2004475 | 0 | 0 | 0 | 0 | 0 | 4.478 | 0 |
| KIDNE2004864 | 0 | 0 | 0 | 0 | 34.453 | 16.985 | 0 |
| KIDNE2006009 | 0 | 0 | 0 | 0 | 0 | 6.414 | 0 |
| KIDNE2006014 | 0 | 0 | 0 | 0 | 10.478 | 5.165 | 0 |
| KIDNE2006039 | 0 | 0 | 0 | 0 | 0 | 27.461 | 0 |
| KIDNE2006248 | 0 | 2.191 | 0 | 0 | 0 | 1.082 | 2.116 |
| KIDNE2006353 | 0 | 0 | 0 | 0 | 0 | 6.803 | 0 |
| KIDNE2006811 | 0 | 22.929 | 0 | 0 | 0 | 0 | 0 |
| KIDNE2007005 | 0 | 0 | 0 | 0 | 0 | 33.289 | 0 |
| KIDNE2007186 | 0 | 0 | 0 | 0 | 0 | 33.289 | 0 |
| KIDNE2007328 | 0 | 0 | 0 | 0 | 0 | 3.078 | 0 |
| KIDNE2007352 | 0 | 0 | 0 | 0 | 4.401 | 3.616 | 0 |
| KIDNE2007811 | 0 | 0 | 0 | 0 | 30.232 | 0 | 0 |
| KIDNE2007910 | 0 | 0 | 0 | 0 | 31.979 | 0 | 0 |
| KIDNE2008048 | 0 | 0 | 0 | 0 | 1.893 | 1.866 | 0 |
| KIDNE2008649 | 0 | 0 | 0 | 0 | 9.333 | 1.534 | 0 |
| KIDNE2008987 | 0 | 0 | 0 | 0 | 10.165 | 0 | 0 |
| KIDNE2010265 | 0 | 0 | 0 | 0 | 0 | 4.110 | 0 |
| KIDNE2012025 | 0 | 0 | 0 | 16.112 | 0 | 0 | 0 |
| KIDNE2013845 | 0 | 0 | 0 | 0 | 0 | 7.777 | 7.606 |
| KIDNE2014119 | 0 | 0 | 0 | 0 | 50.303 | 0 | 0 |
| KIDNE2014290 | 0 | 0 | 0 | 0 | 2.474 | 7.317 | 4.771 |
| KIDNE2015445 | 0 | 0 | 0 | 0 | 12.123 | 5.977 | 0 |
| KIDNE2017343 | 0 | 0 | 0 | 0 | 46.618 | 0 | 0 |
| KIDNE2018352 | 0 | 0 | 0 | 0 | 0 | 6.204 | 0 |
| KIDNE2018493 | 0 | 0 | 0 | 0 | 12.574 | 6.199 | 0 |
| KIDNE2018891 | 0 | 0 | 0 | 0 | 21.519 | 10.609 | 0 |
| LIVER1000132 | 0 | 0 | 0 | 0 | 3.532 | 0.871 | 0 |
| LIVER1000421 | 0 | 0 | 0 | 0 | 28.931 | 0 | 0 |
| LIVER2002842 | 0 | 0 | 0 | 0 | 6.736 | 3.321 | 0 |
| LIVER2005789 | 0 | 0 | 0 | 0 | 0 | 1.566 | 6.126 |
| LIVER2007783 | 0 | 0 | 0 | 0 | 0 | 3.569 | 0 |
| LIVER2008751 | 0 | 0 | 0 | 0 | 0 | 15.105 | 0 |
| MAMGL1000032 | 0 | 0 | 0 | 1.106 | 0.569 | 0.280 | 0 |
| MAMGL1000035 | 0 | 0 | 0 | 0 | 0.711 | 0.350 | 0 |
| MAMGL1000173 | 0 | 0 | 0 | 0 | 0.935 | 0 | 0 |
| MAMGL1000178 | 0 | 0 | 0 | 0 | 0 | 0.532 | 0 |
| MESAN1000126 | 0 | 0 | 0 | 12.261 | 0 | 0 | 0 |
| MESAN2000149 | 0 | 0 | 0 | 0 | 17.337 | 0 | 0 |
| MESAN2000264 | 0 | 0 | 0 | 13.531 | 0 | 0 | 0 |
| MESAN2000267 | 0 | 0 | 0 | 0 | 0 | 1.425 | 0 |
| MESAN2000501 | 0 | 0 | 0 | 0 | 0 | 10.438 | 0 |
| MESAN2000608 | 0 | 0 | 6.130 | 0 | 2.775 | 0 | 0 |
| MESAN2001154 | 0 | 0 | 0 | 9.148 | 0 | 0 | 0 |
| MESAN2002086 | 0 | 0 | 0 | 2.858 | 0 | 2.897 | 0 |
| MESAN2002940 | 0 | 0 | 0 | 21.837 | 0 | 0 | 0 |
| MESAN2003444 | 0 | 0 | 0 | 0 | 0 | 6.480 | 0 |
| MESAN2004288 | 0 | 0 | 40.508 | 0 | 0 | 0 | 0 |
| MESAN2005284 | 0 | 0 | 0 | 0 | 0 | 8.436 | 0 |
| MESAN2005689 | 0 | 0 | 0 | 0 | 0 | 10.794 | 0 |
| MESAN2005958 | 0 | 0 | 0 | 0 | 0 | 9.370 | 0 |
| MESAN2006563 | 0 | 0 | 0 | 4.741 | 2.437 | 0 | 0 |
| MESAN2009081 | 0 | 0 | 0 | 0 | 0 | 8.188 | 0 |
| MESAN2009580 | 0 | 0 | 0 | 0 | 0 | 1.932 | 0 |
| MESAN2013284 | 0 | 0 | 9.574 | 0 | 0 | 0 | 0 |
| NB9N41000135 | 0 | 0 | 0 | 15.228 | 0 | 0 | 0 |
| NB9N42000281 | 0 | 0 | 0 | 0 | 0 | 3.326 | 0 |
| NESOP1000087 | 0 | 0 | 0 | 0 | 14.226 | 0 | 0 |
| NHNPC2000062 | 0 | 0 | 0 | 0 | 14.790 | 7.291 | 0 |
| NHNPC2000206 | 0 | 0 | 0 | 0 | 0 | 1.195 | 0 |
| NOVAR1000015 | 0 | 0 | 0 | 0 | 10.715 | 0 | 0 |
| NOVAR2000038 | 0 | 4.377 | 0 | 0 | 0 | 3.242 | 4.227 |
| NT2NE1000163 | 0 | 0 | 0 | 0 | 0 | 0.705 | 0 |
| NT2NE2000056 | 0 | 0 | 0 | 5.395 | 0 | 0.683 | 0 |
| NT2NE2000174 | 0 | 0 | 0 | 5.240 | 0 | 0 | 0 |
| NT2NE2000259 | 0 | 0 | 0 | 0 | 0 | 4.893 | 0 |
| NT2NE2000260 | 0 | 0 | 0 | 0 | 0.426 | 0 | 0 |
| NT2NE2000383 | 0 | 0 | 0 | 0 | 0 | 2.643 | 0 |
| NT2NE2000470 | 0 | 0 | 42.540 | 0 | 0 | 9.493 | 0 |
| NT2NE2000575 | 0 | 0 | 0 | 0 | 2.545 | 2.927 | 0 |
| NT2NE2000707 | 0 | 0 | 0 | 4.596 | 1.181 | 1.164 | 0 |
| NT2NE2000763 | 0 | 0 | 0 | 0 | 0 | 2.088 | 0 |
| NT2NE2000950 | 0 | 0 | 40.857 | 0 | 0 | 0 | 0 |
| NT2NE2000963 | 0 | 0 | 0 | 0 | 1.619 | 0 | 1.561 |
| NT2NE2001000 | 0 | 0 | 0 | 0 | 0 | 4.491 | 0 |
| NT2NE2001049 | 0 | 0 | 0 | 0 | 0 | 15.287 | 0 |
| NT2NE2001156 | 0 | 0 | 43.812 | 0 | 0 | 9.777 | 0 |
| NT2NE2001181 | 0 | 0 | 0 | 0 | 0 | 32.433 | 0 |
| NT2NE2001617 | 0 | 0 | 0 | 0 | 16.009 | 0 | 0 |
| NT2NE2001793 | 0 | 0 | 0 | 0 | 0 | 1.804 | 0 |
| NT2NE2003185 | 0 | 0 | 0 | 0 | 0 | 6.594 | 0 |
| NT2NE2003485 | 0 | 0 | 0 | 0 | 0 | 4.049 | 0 |
| NT2NE2004519 | 0 | 0 | 0 | 0 | 0 | 6.235 | 0 |
| NT2NE2005371 | 0 | 0 | 0 | 0 | 0 | 0 | 37.523 |
| NT2NE2005441 | 0 | 0 | 0 | 0 | 0 | 0 | 21.143 |
| NT2NE2005688 | 0 | 0 | 0 | 0 | 0 | 1.299 | 0 |
| NT2NE2005876 | 0 | 0 | 0 | 10.128 | 0 | 2.566 | 0 |
| NT2NE2006075 | 0 | 0 | 0 | 0 | 0 | 13.704 | 0 |
| NT2NE2006458 | 0 | 0 | 0 | 0 | 0 | 2.459 | 0 |
| NT2NE2011684 | 0 | 0 | 68.264 | 0 | 0 | 0 | 0 |
| NT2NE2012790 | 0 | 42.270 | 0 | 0 | 0 | 0 | 0 |
| NT2NE2018180 | 0 | 0 | 29.311 | 0 | 0 | 0 | 0 |
| NT2RI1000027 | 0 | 0 | 0 | 0 | 0 | 3.518 | 0 |
| NT2RI2000064 | 0 | 0 | 4.196 | 3.696 | 1.899 | 0.936 | 0 |
| NT2RI2000107 | 0 | 0 | 0 | 0 | 0 | 4.029 | 0 |
| NT2RI2000255 | 0 | 0 | 9.331 | 0 | 4.224 | 0 | 8.145 |
| NT2RI2000263 | 0 | 12.053 | 0 | 0 | 0 | 0 | 0 |
| NT2RI2000282 | 0 | 0 | 0 | 6.520 | 3.351 | 1.101 | 0 |
| NT2RI2000284 | 0 | 0 | 0 | 0 | 1.609 | 3.172 | 0 |
| NT2RI2000412 | 0 | 0 | 0 | 0 | 0 | 13.408 | 0 |
| NT2RI2000671 | 0 | 0 | 0 | 0 | 3.712 | 1.830 | 0 |
| NT2RI2000685 | 0 | 0 | 0 | 0 | 0 | 5.171 | 6.742 |
| NT2RI2000689 | 0 | 0 | 0 | 0 | 0 | 2.690 | 0 |
| NT2RI2000727 | 0 | 0 | 2.017 | 1.776 | 0 | 0.900 | 3.521 |
| NT2RI2001054 | 0 | 0 | 0 | 0 | 0 | 1.035 | 0 |
| NT2RI2001091 | 0 | 0 | 0 | 0 | 12.406 | 0 | 0 |
| NT2RI2001230 | 0 | 0 | 0 | 0 | 0 | 1.342 | 0 |
| NT2RI2001385 | 0 | 0 | 0 | 0 | 0 | 6.854 | 0 |
| NT2RI2001409 | 0 | 0 | 0 | 79.073 | 0 | 0 | 0 |
| NT2RI2001422 | 0 | 0 | 0 | 0 | 13.118 | 0 | 0 |
| NT2RI2001540 | 0 | 1.604 | 1.331 | 1.173 | 2.410 | 1.981 | 2.324 |
| NT2RI2001621 | 0 | 0 | 0 | 0 | 0 | 5.325 | 0 |
| NT2RI2001657 | 0 | 0 | 0 | 0 | 0 | 1.862 | 3.641 |
| NT2RI2001726 | 0 | 0 | 0 | 0 | 0 | 18.369 | 0 |
| NT2RI2001859 | 0 | 0 | 0 | 0 | 0 | 3.376 | 0 |
| NT2RI2002091 | 0 | 0 | 1.046 | 1.473 | 1.373 | 0.677 | 0.639 |
| NT2RI2002117 | 0 | 0 | 0 | 32.348 | 0 | 8.196 | 0 |
| NT2RI2002152 | 0 | 0 | 9.803 | 0 | 0 | 0 | 0 |
| NT2RI2002316 | 0 | 5.649 | 0 | 5.505 | 0 | 1.395 | 0 |
| NT2RI2002391 | 0 | 0 | 0 | 0 | 2.862 | 1.411 | 0 |
| NT2RI2002549 | 0 | 0 | 0 | 47.308 | 0 | 23.972 | 0 |
| NT2RI2002554 | 0 | 0 | 0 | 12.450 | 0 | 6.309 | 0 |
| NT2RI2002847 | 0 | 0 | 9.785 | 0 | 0 | 0 | 0 |
| NT2RI2002958 | 0 | 0 | 0 | 0 | 0 | 1.675 | 0 |
| NT2RI2003205 | 0 | 0 | 0 | 0 | 0 | 0 | 21.906 |
| NT2RI2003304 | 0 | 0 | 0 | 0 | 0.926 | 0 | 1.786 |
| NT2RI2003338 | 0 | 0 | 0 | 0 | 0 | 3.523 | 0 |
| NT2RI2003407 | 0 | 0 | 0 | 0 | 0 | 2.106 | 0 |
| NT2RI2003420 | 0 | 0 | 0 | 0 | 0 | 2.679 | 10.480 |
| NT2RI2003695 | 0 | 0 | 0 | 0 | 0 | 1.370 | 0 |
| NT2RI2003738 | 0 | 0 | 0 | 1.201 | 0 | 1.217 | 0 |
| NT2RI2003921 | 0 | 0 | 0 | 3.256 | 1.674 | 0.825 | 0 |
| NT2RI2004059 | 0 | 0 | 0 | 0 | 0 | 11.854 | 0 |
| NT2RI2004188 | 0 | 0 | 0 | 0 | 0 | 4.995 | 0 |
| NT2RI2004608 | 0 | 0 | 28.841 | 0 | 0 | 0 | 0 |
| NT2RI2005115 | 0 | 0 | 0 | 0 | 0 | 21.261 | 0 |
| NT2RI2005150 | 0 | 0 | 0 | 14.194 | 0 | 1.199 | 0 |
| NT2RI2005239 | 0 | 0 | 0 | 0 | 0 | 6.861 | 0 |
| NT2RI2005405 | 0 | 0 | 0 | 0 | 1.981 | 0.488 | 1.910 |
| NT2RI2005713 | 0 | 0 | 0 | 0 | 0 | 2.573 | 0 |
| NT2RI2005723 | 0 | 3.536 | 0 | 0 | 0 | 0.873 | 0 |
| NT2RI2006070 | 0 | 0 | 0 | 0 | 0 | 0 | 13.986 |
| NT2RI2006072 | 0 | 0 | 0 | 0 | 0 | 1.331 | 0 |
| NT2RI2006412 | 0 | 0 | 0 | 0 | 0 | 0 | 5.578 |
| NT2RI2006825 | 0 | 0 | 0 | 0 | 0 | 0 | 18.657 |
| NT2RI2006853 | 0 | 0 | 0 | 0 | 7.915 | 0 | 30.528 |
| NT2RI2006855 | 0 | 0 | 0 | 0 | 0 | 14.996 | 0 |
| NT2RI2006856 | 0 | 0 | 0 | 3.216 | 0 | 2.445 | 0 |
| NT2RI2007054 | 0 | 9.148 | 0 | 0 | 4.581 | 0 | 8.835 |
| NT2RI2007084 | 0 | 0 | 0 | 0 | 3.839 | 0 | 7.404 |
| NT2RI2007096 | 0 | 0 | 0 | 0 | 0 | 9.018 | 0 |
| NT2RI2007133 | 0 | 0 | 0 | 0 | 1.384 | 0.682 | 2.670 |
| NT2RI2007384 | 0 | 0 | 3.376 | 0 | 4.584 | 1.507 | 2.947 |
| NT2RI2007498 | 0 | 0 | 0 | 0 | 0 | 0 | 2.775 |
| NT2RI2007589 | 0 | 0 | 0 | 7.333 | 0 | 0 | 0 |
| NT2RI2007629 | 0 | 0 | 0 | 0 | 7.113 | 3.507 | 0 |
| NT2RI2007723 | 0 | 0 | 0 | 0 | 0 | 0 | 24.075 |
| NT2RI2007827 | 0 | 0 | 58.521 | 0 | 0 | 0 | 0 |
| NT2RI2007884 | 0 | 0 | 0 | 0 | 0 | 4.416 | 0 |
| NT2RI2007987 | 0 | 0 | 0 | 0 | 0 | 3.132 | 12.253 |
| NT2RI2008045 | 0 | 0 | 6.053 | 0 | 0 | 0 | 0 |
| NT2RI2008171 | 0 | 0 | 0 | 30.488 | 0 | 0 | 0 |
| NT2RI2008204 | 0 | 43.734 | 0 | 0 | 0 | 0 | 0 |
| NT2RI2008221 | 0 | 0 | 0 | 15.225 | 0 | 0 | 0 |
| NT2RI2008336 | 0 | 2.823 | 0 | 2.751 | 0 | 0.697 | 2.726 |
| NT2RI2008481 | 0 | 5.211 | 0 | 0 | 0 | 0 | 0 |
| NT2RI2008526 | 0 | 0 | 11.953 | 0 | 0 | 2.668 | 0 |
| NT2RI2008622 | 0 | 0 | 0 | 0 | 11.136 | 0 | 0 |
| NT2RI2008749 | 0 | 0 | 0 | 0 | 0 | 1.967 | 0 |
| NT2RI2008791 | 0 | 0 | 0 | 0 | 0 | 2.030 | 0 |
| NT2RI2008812 | 0 | 0 | 0 | 0 | 0 | 12.594 | 0 |
| NT2RI2009037 | 0 | 0 | 0 | 0 | 0 | 1.467 | 0 |
| NT2RI2009065 | 0 | 0 | 3.641 | 0 | 0 | 1.625 | 0 |
| NT2RI2009083 | 0 | 0 | 0 | 0 | 0 | 35.675 | 0 |
| NT2RI2009173 | 0 | 0 | 0 | 0 | 0 | 5.821 | 7.590 |
| NT2RI2009239 | 0 | 9.849 | 0 | 0 | 4.932 | 0 | 0 |
| NT2RI2009269 | 0 | 0 | 0 | 0 | 0 | 0 | 13.450 |
| NT2RI2009281 | 0 | 0 | 0 | 0 | 0 | 4.897 | 0 |
| NT2RI2009583 | 0 | 0 | 0 | 0 | 0.790 | 0.778 | 7.613 |
| NT2RI2012726 | 0 | 0 | 0 | 0 | 0 | 8.070 | 0 |
| NT2RI2015533 | 0 | 0 | 0 | 0 | 0 | 2.704 | 0 |
| NT2RI2017529 | 0 | 0 | 0 | 0 | 0 | 3.376 | 0 |
| NT2RI2018448 | 0 | 0 | 0 | 0 | 0 | 10.954 | 0 |
| NT2RI2022468 | 0 | 0 | 0 | 0 | 23.655 | 11.662 | 0 |
| NT2RP6000043 | 0 | 0 | 0 | 0 | 0 | 6.607 | 0 |
| NT2RP6000072 | 0 | 0 | 0 | 0 | 5.702 | 0 | 0 |
| NT2RP6000078 | 0 | 0 | 0 | 0 | 2.538 | 6.255 | 2.447 |
| NT2RP6000123 | 0 | 2.758 | 0 | 0 | 1.381 | 1.362 | 2.664 |
| NT2RP7000076 | 0 | 0 | 4.751 | 0 | 0 | 1.060 | 0 |
| NT2RP7000112 | 0 | 0 | 0 | 0 | 0 | 27.942 | 0 |
| NT2RP7000271 | 0 | 0 | 0 | 0 | 0 | 5.276 | 0 |
| NT2RP7000906 | 0 | 0 | 0 | 0 | 0 | 2.269 | 0 |
| NT2RP7001166 | 0 | 0 | 11.660 | 0 | 0 | 1.301 | 0 |
| NT2RP7001283 | 0 | 0 | 4.388 | 0 | 0 | 0.979 | 0 |
| NT2RP7001306 | 0 | 0 | 56.702 | 0 | 0 | 0 | 0 |
| NT2RP7002282 | 0 | 0 | 0 | 0 | 0 | 26.714 | 0 |
| NT2RP7002379 | 0 | 0 | 0 | 0 | 0 | 0.876 | 0 |
| NT2RP7002449 | 0 | 0 | 0 | 0 | 0 | 3.514 | 0 |
| NT2RP7002619 | 0 | 0 | 0 | 0 | 0 | 1.895 | 0 |
| NT2RP7002829 | 0 | 0 | 0 | 0 | 30.855 | 0 | 0 |
| NT2RP7003261 | 0 | 0 | 10.591 | 0 | 0 | 0 | 0 |
| NT2RP7003688 | 0 | 0 | 5.756 | 0 | 0 | 0 | 0 |
| NT2RP7004173 | 0 | 0 | 0 | 0 | 0 | 5.314 | 0 |
| NT2RP7004348 | 0 | 0 | 0 | 0 | 0 | 3.931 | 0 |
| NT2RP7004373 | 0 | 0 | 0 | 0 | 8.306 | 4.095 | 0 |
| NT2RP7004541 | 0 | 0 | 0 | 0 | 0 | 41.921 | 0 |
| NT2RP7004751 | 0 | 0 | 0 | 3.214 | 0 | 1.628 | 0 |
| NT2RP7004884 | 0 | 0 | 0 | 0 | 9.688 | 0 | 0 |
| NT2RP7004925 | 0 | 0 | 0 | 0 | 0 | 2.197 | 0 |
| NT2RP7004946 | 0 | 0 | 0 | 0 | 3.038 | 1.498 | 0 |
| NT2RP7005513 | 0 | 0 | 0 | 0 | 0 | 2.940 | 0 |
| NT2RP7006075 | 0 | 0 | 0 | 0 | 9.245 | 13.674 | 0 |
| NT2RP7006141 | 0 | 0 | 0 | 0 | 0 | 1.285 | 0 |
| NT2RP7006539 | 0 | 0 | 0 | 0 | 0 | 5.061 | 0 |
| NT2RP7006717 | 0 | 0 | 0 | 0 | 0 | 1.145 | 0 |
| NT2RP7006980 | 0 | 0 | 0 | 0 | 0 | 29.339 | 0 |
| NT2RP7007154 | 0 | 0 | 0 | 0 | 4.024 | 2.976 | 0 |
| NT2RP7007387 | 0 | 0 | 0 | 0 | 0 | 3.571 | 0 |
| NT2RP7007530 | 0 | 0 | 0 | 0 | 7.736 | 0 | 0 |
| NT2RP7007842 | 0 | 0 | 0 | 26.339 | 6.768 | 0 | 0 |
| NT2RP7007975 | 0 | 0 | 0 | 0 | 0 | 2.374 | 0 |
| NT2RP7008167 | 0 | 0 | 0 | 6.232 | 0 | 1.579 | 0 |
| NT2RP7008543 | 0 | 0 | 0 | 0 | 0 | 4.786 | 0 |
| NT2RP7009097 | 0 | 0 | 0 | 0 | 0 | 3.128 | 0 |
| NT2RP7009259 | 0 | 3.959 | 4.380 | 0 | 0 | 0 | 0 |
| NT2RP7009394 | 0 | 0 | 0 | 0 | 0 | 2.104 | 0 |
| NT2RP7009482 | 0 | 0 | 0 | 3.182 | 0 | 0 | 0 |
| NT2RP7012776 | 0 | 0 | 0 | 0 | 0 | 14.621 | 0 |
| NT2RP7014910 | 0 | 0 | 0 | 0 | 0 | 2.080 | 0 |
| NTONG1000033 | 0 | 0 | 0 | 0 | 0 | 4.263 | 0 |
| NTONG1000047 | 0 | 0 | 0 | 0 | 0 | 20.905 | 0 |
| NTONG1000052 | 0 | 6.731 | 0 | 0 | 0 | 0 | 0 |
| NTONG1000075 | 0 | 0 | 0 | 0 | 0 | 0 | 45.341 |
| NTONG1000098 | 0 | 0 | 4.967 | 0 | 0 | 0 | 0 |
| NTONG1000182 | 0 | 0 | 0 | 0 | 0 | 2.154 | 0 |
| NTONG1000246 | 0 | 0 | 12.189 | 0 | 0 | 2.720 | 0 |
| NTONG1000257 | 0 | 0 | 0 | 0 | 0 | 3.829 | 0 |
| NTONG2000265 | 0 | 0 | 0 | 5.853 | 0 | 1.483 | 5.801 |
| NTONG2000858 | 0 | 20.351 | 0 | 0 | 0 | 0 | 0 |
| NTONG62000977 | 0 | 0 | 7.663 | 0 | 0 | 0 | 0 |
| NTONG2001567 | 0 | 0 | 0 | 0 | 2.164 | 0 | 0 |
| NTONG2001612 | 0 | 7.910 | 8.751 | 0 | 0 | 1.953 | 0 |
| NTONG2002582 | 0 | 0 | 0 | 0 | 0 | 6.543 | 25.595 |
| NTONG2003454 | 0 | 0 | 0 | 0 | 10.520 | 0 | 0 |
| NTONG2003515 | 0 | 0 | 0 | 0 | 0 | 17.024 | 0 |
| NTONG2003928 | 0 | 0 | 0 | 0 | 0 | 10.134 | 0 |
| NTONG2004806 | 0 | 48.877 | 0 | 0 | 0 | 0 | 0 |
| NTONG2004844 | 0 | 0 | 0 | 0 | 0 | 2.819 | 0 |
| NTONG2005153 | 0 | 0 | 0 | 0 | 27.921 | 13.765 | 0 |
| NTONG2005520 | 0 | 0 | 0 | 0 | 0 | 1.158 | 4.530 |
| NTONG2005822 | 0 | 0 | 0 | 0 | 32.378 | 0 | 0 |
| NTONG2008483 | 0 | 11.259 | 0 | 0 | 2.819 | 0 | 0 |
| NTONG2008522 | 0 | 0 | 30.549 | 0 | 0 | 0 | 0 |
| NTONG2009468 | 0 | 0 | 0 | 0 | 0 | 16.522 | 0 |
| OCBBF1000086 | 0 | 0 | 0 | 0 | 0 | 0 | 5.209 |
| OCBBF1000114 | 0 | 0 | 0 | 52.512 | 0 | 0 | 0 |
| OCBBF1000146 | 0 | 0 | 0 | 0 | 0 | 4.502 | 0 |
| OCBBF2000013 | 0 | 0 | 15.169 | 0 | 0 | 3.385 | 0 |
| OCBBF2000287 | 0 | 0 | 0 | 0 | 73.962 | 0 | 0 |
| OCBBF2000467 | 0 | 0 | 0 | 0 | 0 | 3.509 | 0 |
| OCBBF2000522 | 0 | 0 | 0 | 0 | 17.846 | 4.399 | 0 |
| OCBBF2000712 | 0 | 0 | 7.367 | 0 | 0 | 0 | 0 |
| OCBBF2000982 | 0 | 0 | 0 | 0 | 0 | 4.551 | 0 |
| OCBBF2001196 | 0 | 0 | 0 | 0 | 55.882 | 0 | 0 |
| OCBBF2001794 | 0 | 0 | 0 | 0 | 16.683 | 0 | 0 |
| OCBBF2002086 | 0 | 0 | 0 | 0 | 0 | 5.605 | 0 |
| OCBBF2002376 | 0 | 0 | 59.348 | 0 | 0 | 0 | 0 |
| OCBBF2004104 | 0 | 0 | 10.677 | 0 | 0 | 2.383 | 0 |
| OCBBF2004235 | 0 | 0 | 0 | 0 | 42.087 | 0 | 0 |
| OCBBF2005956 | 0 | 0 | 0 | 0 | 0 | 4.939 | 0 |
| OCBBF2006660 | 0 | 0 | 0 | 0 | 53.263 | 0 | 0 |
| OCBBF2006849 | 0 | 0 | 0 | 0 | 7.132 | 0 | 0 |
| OCBBF2006859 | 0 | 0 | 58.402 | 0 | 0 | 0 | 0 |
| OCBBF2007354 | 0 | 0 | 0 | 6.898 | 0 | 1.748 | 0 |
| OCBBF2007415 | 0 | 0 | 0 | 10.996 | 0 | 1.393 | 0 |
| OCBBF2007622 | 0 | 0 | 0 | 0 | 12.955 | 0 | 0 |
| OCBBF2008005 | 0 | 0 | 0 | 0 | 5.603 | 0 | 0 |
| OCBBF2008466 | 0 | 0 | 0 | 0 | 0 | 14.882 | 0 |
| OCBBF2009836 | 0 | 0 | 0 | 0 | 0 | 28.513 | 0 |
| OCBBF2010830 | 0 | 0 | 0 | 0 | 3.105 | 1.531 | 0 |
| OCBBF2010841 | 0 | 0 | 0 | 0 | 0 | 32.439 | 0 |
| OCBBF2011536 | 0 | 0 | 0 | 0 | 0 | 3.470 | 0 |
| OCBBF2011914 | 0 | 0 | 0 | 25.037 | 0 | 0 | 0 |
| OCBBF2012553 | 0 | 0 | 0 | 0 | 4.139 | 2.040 | 0 |
| OCBBF2012908 | 0 | 0 | 0 | 0 | 0 | 5.701 | 0 |
| OCBBF2013366 | 0 | 0 | 0 | 0 | 0 | 4.696 | 0 |
| OCBBF2014292 | 0 | 0 | 0 | 0 | 0 | 3.604 | 7.048 |
| OCBBF2014541 | 0 | 0 | 0 | 0 | 0 | 13.642 | 0 |
| OCBBF2015751 | 0 | 0 | 0 | 0 | 0 | 4.930 | 0 |
| OCBBF2016841 | 0 | 0 | 47.620 | 0 | 0 | 0 | 0 |
| OCBBF2016928 | 0 | 0 | 42.696 | 0 | 0 | 0 | 0 |
| OCBBF2017398 | 0 | 0 | 0 | 8.833 | 0 | 0 | 0 |
| OCBBF2017791 | 0 | 0 | 19.969 | 0 | 0 | 0 | 0 |
| OCBBF2018084 | 0 | 0 | 34.374 | 0 | 0 | 0 | 0 |
| OCBBF2018707 | 0 | 0 | 17.532 | 0 | 0 | 0 | 0 |
| OCBBF2021020 | 0 | 0 | 0 | 0 | 0 | 4.638 | 0 |
| OCBBF2035226 | 0 | 0 | 4.580 | 0 | 0 | 1.022 | 0 |
| OCBBF3000323 | 0 | 0 | 0 | 0 | 11.736 | 0 | 0 |
| OCBBF3001333 | 0 | 0 | 0 | 0 | 0 | 5.589 | 0 |
| PEBLM1000034 | 0 | 0 | 0 | 47.739 | 0 | 0 | 0 |
| PEBLM2000126 | 0 | 0 | 0 | 0 | 0 | 18.794 | 0 |
| PEBLM2000222 | 0 | 0 | 0 | 0 | 0 | 0 | 3.953 |
| PEBLM2000395 | 0 | 0 | 0 | 0 | 0 | 1.583 | 0 |
| PEBLM2000479 | 0 | 0 | 0 | 0 | 16.426 | 48.586 | 0 |
| PEBLM2001072 | 0 | 12.778 | 0 | 0 | 0 | 3.155 | 0 |
| PEBLM2004666 | 0 | 0 | 0 | 0 | 0 | 6.464 | 0 |
| PEBLM2007296 | 0 | 0 | 0 | 11.874 | 6.102 | 0 | 0 |
| PEBLM2007774 | 0 | 0 | 0 | 0 | 1.063 | 0.524 | 0 |
| PERIC2000478 | 0 | 0 | 0 | 0 | 0 | 18.939 | 0 |
| PERIC2007068 | 0 | 0 | 0 | 10.595 | 10.890 | 2.684 | 0 |
| PLACE5000013 | 0 | 0 | 0 | 0 | 11.203 | 0 | 0 |
| PLACE5000066 | 0 | 0 | 0 | 0 | 32.287 | 0 | 0 |
| PLACE5000113 | 0 | 0 | 0 | 0 | 0 | 2.993 | 0 |
| PLACE5000170 | 0 | 0 | 0 | 0 | 0 | 12.097 | 0 |
| PLACE6000371 | 0 | 0 | 0 | 0 | 0 | 49.431 | 0 |
| PLACE6000379 | 0 | 0 | 0 | 0 | 1.870 | 0.307 | 1.202 |
| PLACE6000414 | 0 | 0 | 0 | 0 | 4.522 | 2.229 | 8.720 |
| PLACE6000463 | 0 | 0 | 0 | 0 | 0 | 13.574 | 0 |
| PLACE6000630 | 0 | 0 | 0 | 0 | 0 | 6.713 | 0 |
| PLACE6001064 | 0 | 0 | 0 | 0 | 2.024 | 0 | 0 |
| PLACE6001443 | 0 | 0 | 0 | 0 | 6.931 | 0 | 13.367 |
| PLACE6001886 | 0 | 0 | 0 | 0 | 0 | 29.462 | 0 |
| PLACE6002011 | 0 | 0 | 0 | 0 | 0 | 49.431 | 0 |
| PLACE6002056 | 0 | 0 | 0 | 0 | 0 | 49.431 | 0 |
| PLACE6002084 | 0 | 0 | 0 | 0 | 0 | 3.855 | 0 |
| PLACE6002419 | 0 | 0 | 0 | 0 | 0 | 0 | 70.943 |
| PLACE6002668 | 0 | 0 | 0 | 0 | 0 | 9.388 | 0 |
| PLACE6002960 | 0 | 0 | 0 | 0 | 0 | 1.931 | 7.553 |
| PLACE6003383 | 0 | 8.410 | 0 | 0 | 0 | 0 | 0 |
| PLACE6003850 | 0 | 0 | 0 | 0 | 39.586 | 0 | 0 |
| PLACE6004005 | 0 | 0 | 0 | 6.779 | 0 | 0 | 0 |
| PLACE6004312 | 0 | 0 | 0 | 0 | 4.256 | 2.098 | 0 |
| PLACE6004464 | 0 | 0 | 0 | 0 | 0 | 32.829 | 0 |
| PLACE6004578 | 0 | 0 | 0 | 0 | 4.374 | 0 | 0 |
| PLACE6006042 | 0 | 0 | 0 | 0 | 19.210 | 0 | 0 |
| PLACE6006418 | 0 | 0 | 0 | 0 | 9.783 | 0 | 0 |
| PLACE6006549 | 0 | 0 | 0 | 0 | 2.818 | 0 | 0 |
| PLACE6008775 | 0 | 0 | 0 | 0 | 6.504 | 0 | 0 |
| PLACE6008793 | 0 | 0 | 12.360 | 0 | 0 | 5.516 | 0 |
| PLACE6009228 | 0 | 0 | 0 | 0 | 0 | 49.431 | 0 |
| PLACE6010077 | 0 | 0 | 6.787 | 0 | 0 | 1.515 | 0 |
| PLACE6010484 | 0 | 0 | 0 | 0 | 5.454 | 5.378 | 0 |
| PLACE6015322 | 0 | 0 | 0 | 0 | 0 | 49.431 | 0 |
| PLACE6016254 | 0 | 0 | 0 | 0 | 0 | 31.784 | 0 |
| PLACE6016942 | 0 | 0 | 0 | 0 | 0 | 6.805 | 0 |
| PROST1000097 | 0 | 4.143 | 0 | 0 | 2.075 | 2.046 | 0 |
| PROST1000120 | 0 | 0 | 0 | 0 | 0 | 5.737 | 0 |
| PROST1000144 | 0 | 0 | 0 | 10.765 | 0 | 0 | 0 |
| PROST1000199 | 0 | 0 | 0 | 0 | 0 | 2.542 | 0 |
| PROST1000215 | 0 | 0 | 0 | 0 | 0 | 17.795 | 0 |
| PROST1000226 | 0 | 0 | 0 | 0 | 6.314 | 18.675 | 0 |
| PROST1000246 | 0 | 0 | 0 | 0 | 38.770 | 0 | 0 |
| PROST1000480 | 0 | 0 | 0 | 0 | 0 | 32.910 | 0 |
| PROST1000526 | 0 | 0 | 19.695 | 0 | 0 | 0 | 0 |
| PROST1000564 | 0 | 0 | 10.479 | 0 | 4.744 | 0 | 0 |
| PROST2000138 | 0 | 0 | 0.676 | 0.595 | 2.753 | 0.302 | 1.180 |
| PROST2001180 | 0 | 0 | 0 | 0 | 8.762 | 4.319 | 0 |
| PROST2001283 | 0 | 14.993 | 0 | 0 | 0 | 0 | 0 |
| PROST2001552 | 0 | 0 | 0 | 0 | 0 | 0 | 5.924 |
| PROST2001739 | 0 | 0 | 0 | 0 | 11.664 | 0 | 0 |
| PROST2001883 | 0 | 0 | 6.962 | 6.133 | 0 | 0 | 0 |
| PROST2002078 | 0 | 0 | 0 | 0 | 8.576 | 0 | 0 |
| PROST2002425 | 0 | 0 | 0 | 0 | 0 | 27.641 | 0 |
| PROST2003232 | 0 | 0 | 0 | 0 | 0 | 2.374 | 0 |
| PROST2003338 | 0 | 0 | 0 | 0 | 0 | 1.947 | 0 |
| PROST2003428 | 0 | 0 | 0 | 0 | 0 | 32.910 | 0 |
| PROST2004095 | 0 | 0 | 0 | 0 | 0 | 0.814 | 0 |
| PROST2004258 | 0 | 0 | 0 | 0 | 0 | 6.637 | 0 |
| PROST2004270 | 0 | 0 | 0 | 0 | 0 | 1.023 | 0 |
| PROST2004416 | 0 | 4.791 | 0 | 0 | 0 | 5.914 | 0 |
| PROST2005143 | 0 | 0 | 0 | 0 | 0 | 11.801 | 0 |
| PROST2005272 | 0 | 0 | 0 | 0 | 28.978 | 14.286 | 0 |
| PROST2006282 | 0 | 0 | 0 | 0 | 62.691 | 0 | 0 |
| PROST2006933 | 0 | 0 | 0 | 0 | 0 | 32.910 | 0 |
| PROST2008360 | 0 | 0 | 0 | 0 | 0 | 0 | 12.248 |
| PROST2009320 | 0 | 0 | 0 | 0 | 49.876 | 0 | 0 |
| PROST2010545 | 0 | 0 | 0 | 0 | 0 | 22.894 | 0 |
| PROST2012745 | 0 | 0 | 0 | 0 | 0 | 0 | 26.318 |
| PROST2012780 | 0 | 0 | 0 | 0 | 25.670 | 0 | 0 |
| PROST2013053 | 0 | 0 | 0 | 0 | 0 | 0 | 20.156 |
| PROST2014862 | 0 | 0 | 0 | 0 | 0 | 27.641 | 0 |
| PROST2015464 | 0 | 0 | 0 | 0 | 6.052 | 2.984 | 0 |
| PROST2016980 | 0 | 0 | 0 | 0 | 0 | 19.696 | 0 |
| PROST2017692 | 0 | 0 | 0 | 5.060 | 0 | 0 | 5.015 |
| PROST2017729 | 0 | 0 | 0 | 0 | 49.876 | 0 | 0 |
| PROST2019164 | 0 | 25.982 | 0 | 0 | 0 | 0 | 0 |
| PROST2019253 | 0 | 0 | 0 | 0 | 0 | 2.923 | 0 |
| PUAEN1000065 | 0 | 0 | 0 | 0 | 0 | 2.330 | 4.558 |
| PUAEN1000164 | 0 | 0 | 0 | 0 | 0 | 0.692 | 0 |
| PUAEN1000322 | 0 | 0 | 19.931 | 0 | 0 | 0 | 0 |
| PUAEN2000535 | 0 | 0 | 6.269 | 0 | 0 | 0 | 0 |
| PUAEN2002568 | 0 | 0 | 0 | 0 | 38.476 | 0 | 0 |
| PUAEN2005502 | 0 | 0 | 0 | 0 | 0 | 18.619 | 0 |
| RECTM2000510 | 0 | 0 | 23.496 | 0 | 0 | 0 | 0 |
| SALGL1000005 | 97.831 | 0 | 0 | 0 | 0 | 0 | 0 |
| SALGL1000024 | 98.036 | 0 | 0 | 0 | 0 | 0 | 0 |
| SALGL1000028 | 100 | 0 | 0 | 0 | 0 | 0 | 0 |
| SALGL1000047 | 95.567 | 0 | 0 | 0 | 0 | 0 | 0 |
| SALGL1000065 | 100 | 0 | 0 | 0 | 0 | 0 | 0 |
| SALGL1000107 | 99.596 | 0 | 0 | 0 | 0 | 0 | 0 |
| SALGL1000157 | 41.950 | 0 | 0 | 0 | 0 | 0.905 | 0 |
| SALGL1000171 | 99.486 | 0 | 0 | 0 | 0 | 0 | 0 |
| SKMUS1000014 | 0 | 51.833 | 0 | 0 | 0 | 0 | 0 |
| SKMUS1000022 | 0 | 2.933 | 3.245 | 0 | 1.469 | 0 | 0 |
| SKMUS1000030 | 0 | 100 | 0 | 0 | 0 | 0 | 0 |
| SKMUS1000064 | 0 | 36.376 | 0 | 0 | 0 | 0 | 0 |
| SKMUS1000067 | 0 | 100 | 0 | 0 | 0 | 0 | 0 |
| SKMUS1000084 | 0 | 10.852 | 0 | 0 | 0 | 0 | 0 |
| SKMUS1000102 | 0 | 68.143 | 0 | 0 | 0 | 0 | 0 |
| SKMUS1000104 | 0 | 100 | 0 | 0 | 0 | 0 | 0 |
| SKMUS1000110 | 0 | 51.909 | 0 | 0 | 0 | 0 | 0 |
| SKMUS1000118 | 0 | 100 | 0 | 0 | 0 | 0 | 0 |
| SKMUS1000124 | 0 | 100 | 0 | 0 | 0 | 0 | 0 |
| SKMUS1000129 | 0 | 100 | 0 | 0 | 0 | 0 | 0 |
| SKMUS1000138 | 0 | 34.510 | 0 | 0 | 0 | 0 | 0 |
| SKMUS1000176 | 0 | 6.079 | 0 | 0 | 0 | 0 | 0 |
| SKMUS1000177 | 0 | 16.639 | 0 | 0 | 0 | 0 | 0 |
| SKMUS1000186 | 0 | 2.828 | 0 | 0 | 1.416 | 0 | 0 |
| SKMUS2000020 | 0 | 25.952 | 0 | 0 | 0 | 0 | 0 |
| SKMUS2000061 | 0 | 100 | 0 | 0 | 0 | 0 | 0 |
| SKMUS2000074 | 0 | 72.072 | 0 | 0 | 0 | 0 | 0 |
| SKMUS2000078 | 0 | 100 | 0 | 0 | 0 | 0 | 0 |
| SKMUS2000117 | 0 | 59.874 | 0 | 0 | 0 | 0 | 0 |
| SKMUS2000137 | 0 | 100 | 0 | 0 | 0 | 0 | 0 |
| SKMUS2000198 | 0 | 55.645 | 0 | 0 | 0 | 0 | 0 |
| SKMUS2000243 | 0 | 71.082 | 0 | 0 | 0 | 0 | 0 |
| SKMUS2000271 | 0 | 44.946 | 0 | 0 | 0 | 0 | 0 |
| SKMUS2000287 | 0 | 100 | 0 | 0 | 0 | 0 | 0 |
| SKMUS2000317 | 0 | 100 | 0 | 0 | 0 | 0 | 0 |
| SKMUS2000330 | 0 | 100 | 0 | 0 | 0 | 0 | 0 |
| SKMUS2000339 | 0 | 100 | 0 | 0 | 0 | 0 | 0 |
| SKMUS2000343 | 0 | 54.290 | 0 | 0 | 0 | 0 | 0 |
| SKMUS2000361 | 0 | 7.535 | 0 | 0 | 0 | 0 | 0 |
| SKMUS2000365 | 0 | 100 | 0 | 0 | 0 | 0 | 0 |
| SKMUS2000380 | 0 | 45.841 | 0 | 0 | 0 | 3.773 | 0 |
| SKMUS2000390 | 0 | 100 | 0 | 0 | 0 | 0 | 0 |
| SKMUS2000416 | 0 | 100 | 0 | 0 | 0 | 0 | 0 |
| SKMUS2000458 | 0 | 80.810 | 0 | 0 | 0 | 0 | 0 |
| SKMUS2000467 | 0 | 79.497 | 0 | 0 | 0 | 0 | 0 |
| SKMUS2000468 | 0 | 100 | 0 | 0 | 0 | 0 | 0 |
| SKMUS2000484 | 0 | 12.373 | 0 | 0 | 0 | 0 | 0 |
| SKMUS2000515 | 0 | 79.836 | 0 | 0 | 0 | 0 | 0 |
| SKMUS2000679 | 0 | 23.603 | 0 | 0 | 0 | 0 | 0 |
| SKMUS2000690 | 0 | 34.321 | 0 | 0 | 0 | 0 | 0 |
| SKMUS2000701 | 0 | 100 | 0 | 0 | 0 | 0 | 0 |
| SKMUS2000705 | 0 | 100 | 0 | 0 | 0 | 0 | 0 |
| SKMUS2000710 | 0 | 100 | 0 | 0 | 0 | 0 | 0 |
| SKMUS2000724 | 0 | 100 | 0 | 0 | 0 | 0 | 0 |
| SKMUS2000726 | 0 | 100 | 0 | 0 | 0 | 0 | 0 |
| SKMUS2000757 | 0 | 34.821 | 0 | 0 | 0 | 8.597 | 0 |
| SKMUS2000774 | 0 | 100 | 0 | 0 | 0 | 0 | 0 |
| SKMUS2000780 | 0 | 67.038 | 0 | 0 | 0 | 0 | 0 |
| SKMUS2000821 | 0 | 78.301 | 0 | 0 | 0 | 0 | 0 |
| SKMUS2000847 | 0 | 100 | 0 | 0 | 0 | 0 | 0 |
| SKMUS2000863 | 0 | 100 | 0 | 0 | 0 | 0 | 0 |
| SKMUS2000873 | 0 | 15.504 | 0 | 0 | 1.941 | 0 | 0 |
| SKMUS2000894 | 0 | 100 | 0 | 0 | 0 | 0 | 0 |
| SKMUS2000898 | 0 | 100 | 0 | 0 | 0 | 0 | 0 |
| SKMUS2000902 | 0 | 38.110 | 0 | 0 | 0 | 0 | 0 |
| SKMUS2000931 | 0 | 7.668 | 4.242 | 3.736 | 1.920 | 0 | 0 |
| SKMUS2000933 | 0 | 43.718 | 0 | 0 | 0 | 0 | 0 |
| SKMUS2000945 | 0 | 100 | 0 | 0 | 0 | 0 | 0 |
| SKMUS2000954 | 0 | 35.928 | 0 | 0 | 5.997 | 0 | 0 |
| SKMUS2000961 | 0 | 46.388 | 0 | 0 | 0 | 0 | 0 |
| SKMUS2000973 | 0 | 100 | 0 | 0 | 0 | 0 | 0 |
| SKMUS2000980 | 0 | 100 | 0 | 0 | 0 | 0 | 0 |
| SKMUS2001008 | 0 | 90.149 | 0 | 0 | 0 | 0 | 0 |
| SKMUS2001014 | 0 | 100 | 0 | 0 | 0 | 0 | 0 |
| SKMUS2001129 | 0 | 35.832 | 0 | 0 | 0 | 0 | 0 |
| SKMUS2001147 | 0 | 39.065 | 0 | 0 | 0 | 0 | 0 |
| SKMUS2001157 | 0 | 75.835 | 0 | 0 | 0 | 0 | 0 |
| SKMUS2001164 | 0 | 100 | 0 | 0 | 0 | 0 | 0 |
| SKMUS2001199 | 0 | 100 | 0 | 0 | 0 | 0 | 0 |
| SKMUS2001201 | 0 | 50.208 | 0 | 0 | 0 | 0 | 0 |
| SKMUS2001215 | 0 | 100 | 0 | 0 | 0 | 0 | 0 |
| SKMUS2001247 | 0 | 100 | 0 | 0 | 0 | 0 | 0 |
| SKMUS2001254 | 0 | 87.489 | 0 | 0 | 0 | 0 | 0 |
| SKMUS2001271 | 0 | 100 | 0 | 0 | 0 | 0 | 0 |
| SKMUS2001287 | 0 | 100 | 0 | 0 | 0 | 0 | 0 |
| SKMUS2001323 | 0 | 12.378 | 0 | 0 | 0 | 0 | 0 |
| SKMUS2001364 | 0 | 100 | 0 | 0 | 0 | 0 | 0 |
| SKMUS2001398 | 0 | 100 | 0 | 0 | 0 | 0 | 0 |
| SKMUS2001402 | 0 | 29.705 | 0 | 0 | 0 | 0 | 0 |
| SKMUS2001412 | 0 | 87.489 | 0 | 0 | 0 | 0 | 0 |
| SKMUS2001454 | 0 | 2.378 | 0 | 0 | 2.382 | 0.587 | 0 |
| SKMUS2001492 | 0 | 100 | 0 | 0 | 0 | 0 | 0 |
| SKMUS2001501 | 0 | 61.433 | 0 | 0 | 0 | 3.792 | 0 |
| SKMUS2001528 | 0 | 8.844 | 0 | 0 | 0 | 0 | 0 |
| SKMUS2001543 | 0 | 76.794 | 0 | 0 | 0 | 0 | 0 |
| SKMUS2001580 | 0 | 100 | 0 | 0 | 0 | 0 | 0 |
| SKMUS2001585 | 0 | 100 | 0 | 0 | 0 | 0 | 0 |
| SKMUS2001608 | 0 | 100 | 0 | 0 | 0 | 0 | 0 |
| SKMUS2001631 | 0 | 100 | 0 | 0 | 0 | 0 | 0 |
| SKMUS2001634 | 0 | 9.225 | 0 | 0 | 0 | 4.555 | 0 |
| SKMUS2001662 | 0 | 100 | 0 | 0 | 0 | 0 | 0 |
| SKMUS2001663 | 0 | 100 | 0 | 0 | 0 | 0 | 0 |
| SKMUS2001668 | 0 | 80.039 | 0 | 0 | 0 | 0 | 0 |
| SKMUS2001671 | 0 | 100 | 0 | 0 | 0 | 0 | 0 |
| SKMUS2001683 | 0 | 100 | 0 | 0 | 0 | 0 | 0 |
| SKMUS2001693 | 0 | 67.659 | 0 | 0 | 0 | 0 | 0 |
| SKMUS2001695 | 0 | 63.549 | 0 | 0 | 0 | 0 | 0 |
| SKMUS2001703 | 0 | 100 | 0 | 0 | 0 | 0 | 0 |
| SKMUS2001740 | 0 | 100 | 0 | 0 | 0 | 0 | 0 |
| SKMUS2001742 | 0 | 100 | 0 | 0 | 0 | 0 | 0 |
| SKMUS2001823 | 0 | 100 | 0 | 0 | 0 | 0 | 0 |
| SKMUS2001850 | 0 | 100 | 0 | 0 | 0 | 0 | 0 |
| SKMUS2002077 | 0 | 100 | 0 | 0 | 0 | 0 | 0 |
| SKMUS2002084 | 0 | 43.995 | 0 | 0 | 0 | 0 | 0 |
| SKMUS2002153 | 0 | 100 | 0 | 0 | 0 | 0 | 0 |
| SKMUS2002475 | 0 | 100 | 0 | 0 | 0 | 0 | 0 |
| SKMUS2002602 | 0 | 100 | 0 | 0 | 0 | 0 | 0 |
| SKMUS2002634 | 0 | 36.218 | 0 | 0 | 0 | 0 | 0 |
| SKMUS2002693 | 0 | 100 | 0 | 0 | 0 | 0 | 0 |
| SKMUS2002821 | 0 | 100 | 0 | 0 | 0 | 0 | 0 |
| SKMUS2002840 | 0 | 100 | 0 | 0 | 0 | 0 | 0 |
| SKMUS2002920 | 0 | 100 | 0 | 0 | 0 | 0 | 0 |
| SKMUS2003034 | 0 | 100 | 0 | 0 | 0 | 0 | 0 |
| SKMUS2003074 | 0 | 100 | 0 | 0 | 0 | 0 | 0 |
| SKMUS2003168 | 0 | 66.520 | 0 | 0 | 0 | 0 | 0 |
| SKMUS2003194 | 0 | 91.511 | 0 | 0 | 0 | 0 | 0 |
| SKMUS2003744 | 0 | 100 | 0 | 0 | 0 | 0 | 0 |
| SKMUS2004044 | 0 | 91.511 | 0 | 0 | 0 | 0 | 0 |
| SKMUS2004047 | 0 | 100 | 0 | 0 | 0 | 0 | 0 |
| SKMUS2004483 | 0 | 100 | 0 | 0 | 0 | 0 | 0 |
| SKMUS2004667 | 0 | 66.520 | 0 | 0 | 0 | 0 | 0 |
| SKMUS2004897 | 0 | 100 | 0 | 0 | 0 | 0 | 0 |
| SKMUS2004903 | 0 | 51.125 | 0 | 0 | 0 | 0 | 0 |
| SKMUS2004908 | 0 | 23.145 | 0 | 0 | 0 | 0 | 0 |
| SKMUS2005428 | 0 | 100 | 0 | 0 | 0 | 0 | 0 |
| SKMUS2006394 | 0 | 67.870 | 0 | 0 | 0 | 0 | 0 |
| SKMUS2006481 | 0 | 100 | 0 | 0 | 0 | 0 | 0 |
| SKMUS2006755 | 0 | 51.125 | 0 | 0 | 0 | 0 | 0 |
| SKMUS2007315 | 0 | 100 | 0 | 0 | 0 | 0 | 0 |
| SKMUS2007359 | 0 | 100 | 0 | 0 | 0 | 0 | 0 |
| SKMUS2007568 | 0 | 100 | 0 | 0 | 0 | 0 | 0 |
| SKMUS2007740 | 0 | 100 | 0 | 0 | 0 | 0 | 0 |
| SKMUS2007816 | 0 | 100 | 0 | 0 | 0 | 0 | 0 |
| SKMUS2007915 | 0 | 100 | 0 | 0 | 0 | 0 | 0 |
| SKMUS2008338 | 0 | 100 | 0 | 0 | 0 | 0 | 0 |
| SKMUS2008407 | 0 | 100 | 0 | 0 | 0 | 0 | 0 |
| SKMUS2008474 | 0 | 100 | 0 | 0 | 0 | 0 | 0 |
| SKMUS2008585 | 0 | 100 | 0 | 0 | 0 | 0 | 0 |
| SKMUS2008607 | 0 | 100 | 0 | 0 | 0 | 0 | 0 |
| SKMUS2009190 | 0 | 100 | 0 | 0 | 0 | 0 | 0 |
| SKMUS2009232 | 0 | 100 | 0 | 0 | 0 | 0 | 0 |
| SKMUS2009479 | 0 | 100 | 0 | 0 | 0 | 0 | 0 |
| SKMUS2009557 | 0 | 100 | 0 | 0 | 0 | 0 | 0 |
| SKNMC1000110 | 0 | 0 | 28.223 | 0 | 0 | 0 | 0 |
| SKNMC1000123 | 0 | 0 | 100 | 0 | 0 | 0 | 0 |
| SKNMC1000124 | 0 | 0 | 100 | 0 | 0 | 0 | 0 |
| SKNMC1000137 | 0 | 0 | 8.264 | 0 | 3.741 | 1.844 | 0 |
| SKNMC1000159 | 0 | 0 | 82.323 | 0 | 0 | 0 | 0 |
| SKNMC1000168 | 0 | 0 | 6.528 | 0 | 0 | 0 | 0 |
| SKNMC1000229 | 0 | 0 | 40.981 | 0 | 0 | 0 | 0 |
| SKNMC1000251 | 0 | 0 | 54.679 | 0 | 0 | 0 | 0 |
| SKNMC1000264 | 0 | 0 | 40.887 | 0 | 0 | 0 | 0 |
| SKNMC2000065 | 0 | 0 | 8.415 | 0 | 0 | 0 | 0 |
| SKNMC2000097 | 0 | 0 | 6.949 | 0 | 0 | 0.775 | 0 |
| SKNMC2000224 | 0 | 0 | 48.830 | 0 | 0 | 0 | 0 |
| SKNMC2000256 | 0 | 0 | 100 | 0 | 0 | 0 | 0 |
| SKNMC2000305 | 0 | 0 | 27.458 | 0 | 0 | 0 | 0 |
| SKNMC2000322 | 0 | 0 | 11.009 | 0 | 4.983 | 0 | 0 |
| SKNMC2000356 | 0 | 0 | 68.922 | 0 | 0 | 0 | 0 |
| SKNMC2000374 | 0 | 0 | 100 | 0 | 0 | 0 | 0 |
| SKNMC2000583 | 0 | 0 | 100 | 0 | 0 | 0 | 0 |
| SKNMC2000593 | 0 | 0 | 68.004 | 0 | 0 | 0 | 0 |
| SKNMC2000612 | 0 | 0 | 100 | 0 | 0 | 0 | 0 |
| SKNMC2000622 | 0 | 0 | 100 | 0 | 0 | 0 | 0 |
| SKNMC2000635 | 0 | 0 | 1.398 | 0 | 0 | 0 | 0 |
| SKNMC2000649 | 0 | 0 | 7.112 | 0 | 0 | 1.587 | 0 |
| SKNMC2000698 | 0 | 0 | 16.101 | 0 | 0 | 0 | 0 |
| SKNMC2000877 | 0 | 0 | 9.237 | 0 | 8.363 | 0 | 0 |
| SKNMC2000966 | 0 | 0 | 63.124 | 0 | 0 | 0 | 0 |
| SKNMC2001113 | 0 | 0 | 76.091 | 0 | 0 | 16.981 | 0 |
| SKNMC2001324 | 0 | 0 | 50.339 | 0 | 0 | 0 | 0 |
| SKNMC2001503 | 0 | 0 | 24.175 | 21.294 | 0 | 0 | 0 |
| SKNMC2001555 | 0 | 0 | 100 | 0 | 0 | 0 | 0 |
| SKNMC2001596 | 0 | 0 | 100 | 0 | 0 | 0 | 0 |
| SKNMC2001623 | 0 | 0 | 78.328 | 0 | 0 | 0 | 0 |
| SKNMC2002402 | 0 | 0 | 57.469 | 0 | 0 | 12.825 | 0 |
| SKNMC2003639 | 0 | 0 | 80.715 | 0 | 0 | 0 | 0 |
| SKNMC2003924 | 0 | 0 | 100 | 0 | 0 | 0 | 0 |
| SKNMC2003987 | 0 | 0 | 100 | 0 | 0 | 0 | 0 |
| SKNMC2004457 | 0 | 0 | 100 | 0 | 0 | 0 | 0 |
| SKNMC2004643 | 0 | 0 | 100 | 0 | 0 | 0 | 0 |
| SKNMC2004651 | 0 | 0 | 100 | 0 | 0 | 0 | 0 |
| SKNMC2005772 | 0 | 0 | 100 | 0 | 0 | 0 | 0 |
| SKNMC2006173 | 0 | 0 | 59.986 | 0 | 0 | 0 | 0 |
| SKNMC2006327 | 0 | 0 | 100 | 0 | 0 | 0 | 0 |
| SKNMC2006998 | 0 | 0 | 100 | 0 | 0 | 0 | 0 |
| SKNMC2007134 | 0 | 0 | 100 | 0 | 0 | 0 | 0 |
| SKNMC2007502 | 0 | 0 | 100 | 0 | 0 | 0 | 0 |
| SKNMC2007504 | 0 | 0 | 100 | 0 | 0 | 0 | 0 |
| SKNMC2007961 | 0 | 0 | 100 | 0 | 0 | 0 | 0 |
| SKNMC2009450 | 0 | 0 | 100 | 0 | 0 | 0 | 0 |
| SKNSH1000002 | 0 | 0 | 0 | 9.869 | 0 | 0 | 0 |
| SKNSH1000086 | 0 | 0 | 0 | 58.629 | 0 | 0 | 0 |
| SKNSH1000101 | 0 | 0 | 0 | 4.411 | 0 | 0 | 4.371 |
| SKNSH1000174 | 0 | 0 | 0 | 100 | 0 | 0 | 0 |
| SKNSH1000301 | 0 | 0 | 0 | 100 | 0 | 0 | 0 |
| SKNSH1000308 | 0 | 0 | 0 | 100 | 0 | 0 | 0 |
| SKNSH1000416 | 0 | 0 | 0 | 100 | 0 | 0 | 0 |
| SKNSH2000051 | 0 | 0 | 0 | 58.015 | 0 | 0 | 0 |
| SKNSH2000101 | 0 | 0 | 0 | 45.247 | 0 | 0 | 0 |
| SKNSH2000151 | 0 | 0 | 0 | 37.354 | 0 | 0 | 0 |
| SKNSH2000163 | 0 | 0 | 0 | 72.863 | 0 | 0 | 0 |
| SKNSH2000250 | 0 | 0 | 0 | 9.674 | 0 | 0 | 0 |
| SKNSH2000319 | 0 | 0 | 0 | 49.241 | 0 | 0 | 0 |
| SKNSH2000347 | 0 | 0 | 0 | 15.406 | 0 | 0 | 0 |
| SKNSH2000482 | 0 | 0 | 0 | 66.928 | 0 | 0 | 0 |
| SKNSH2000516 | 0 | 0 | 0 | 100 | 0 | 0 | 0 |
| SKNSH2000550 | 0 | 0 | 0 | 100 | 0 | 0 | 0 |
| SKNSH2000716 | 0 | 0 | 0 | 11.900 | 0 | 0 | 0 |
| SKNSH2000819 | 0 | 0 | 19.403 | 17.090 | 0 | 0 | 0 |
| SKNSH2000971 | 0 | 0 | 0 | 49.371 | 0 | 0 | 0 |
| SKNSH2001222 | 0 | 0 | 0 | 44.836 | 0 | 0 | 0 |
| SKNSH2001875 | 0 | 0 | 0 | 44.586 | 0 | 11.296 | 0 |
| SKNSH2001931 | 0 | 0 | 0 | 35.089 | 0 | 0 | 0 |
| SKNSH2002054 | 0 | 0 | 0 | 100 | 0 | 0 | 0 |
| SKNSH2002325 | 0 | 0 | 0 | 26.212 | 0 | 0 | 0 |
| SKNSH2002768 | 0 | 0 | 0 | 16.257 | 0 | 8.238 | 0 |
| SKNSH2002866 | 0 | 0 | 0 | 100 | 0 | 0 | 0 |
| SKNSH2003064 | 0 | 0 | 0 | 53.753 | 0 | 0 | 0 |
| SKNSH2003174 | 0 | 0 | 0 | 100 | 0 | 0 | 0 |
| SKNSH2003422 | 0 | 0 | 0 | 100 | 0 | 0 | 0 |
| SKNSH2003466 | 0 | 0 | 0 | 100 | 0 | 0 | 0 |
| SKNSH2003483 | 0 | 0 | 0 | 100 | 0 | 0 | 0 |
| SKNSH2003528 | 0 | 0 | 0 | 20.844 | 0 | 0 | 0 |
| SKNSH2003633 | 0 | 0 | 0 | 46.056 | 0 | 0 | 0 |
| SKNSH2004039 | 0 | 0 | 0 | 100 | 0 | 0 | 0 |
| SKNSH2005061 | 0 | 0 | 0 | 11.640 | 0 | 0 | 0 |
| SKNSH2005120 | 0 | 0 | 0 | 100 | 0 | 0 | 0 |
| SKNSH2005194 | 0 | 0 | 0 | 100 | 0 | 0 | 0 |
| SKNSH2005240 | 0 | 0 | 0 | 91.307 | 0 | 0 | 0 |
| SKNSH2005792 | 0 | 0 | 0 | 100 | 0 | 0 | 0 |
| SKNSH2005816 | 0 | 0 | 0 | 100 | 0 | 0 | 0 |
| SKNSH2005981 | 0 | 0 | 0 | 100 | 0 | 0 | 0 |
| SKNSH2006234 | 0 | 0 | 0 | 24.035 | 0 | 0 | 0 |
| SKNSH2006304 | 0 | 0 | 0 | 100 | 0 | 0 | 0 |
| SKNSH2006432 | 0 | 0 | 0 | 100 | 0 | 0 | 0 |
| SKNSH2006822 | 0 | 0 | 0 | 100 | 0 | 0 | 0 |
| SKNSH2007142 | 0 | 0 | 0 | 100 | 0 | 0 | 0 |
| SKNSH2007149 | 0 | 0 | 0 | 100 | 0 | 0 | 0 |
| SKNSH2007429 | 0 | 0 | 0 | 100 | 0 | 0 | 0 |
| SKNSH2007739 | 0 | 0 | 0 | 100 | 0 | 0 | 0 |
| SKNSH2008043 | 0 | 0 | 0 | 69.524 | 0 | 0 | 0 |
| SKNSH2008940 | 0 | 0 | 0 | 13.166 | 0 | 0 | 0 |
| SKNSH2008969 | 0 | 0 | 0 | 100 | 0 | 0 | 0 |
| SKNSH2009197 | 0 | 0 | 0 | 100 | 0 | 0 | 0 |
| SKNSH2009357 | 0 | 0 | 0 | 65.592 | 0 | 0 | 0 |
| SKNSH2009435 | 0 | 0 | 0 | 100 | 0 | 0 | 0 |
| SKNSH2009991 | 0 | 0 | 0 | 100 | 0 | 0 | 0 |
| SKNSH2010015 | 0 | 0 | 0 | 100 | 0 | 0 | 0 |
| SMINT1000012 | 0 | 0 | 0 | 0 | 100 | 0 | 0 |
| SMINT1000016 | 0 | 0 | 0 | 0 | 8.657 | 0 | 0 |
| SMINT1000039 | 0 | 0 | 0 | 0 | 34.704 | 0 | 0 |
| SMINT1000042 | 0 | 0 | 0 | 0 | 9.454 | 0 | 0 |
| SMINT1000048 | 0 | 0 | 0 | 0 | 27.794 | 27.404 | 0 |
| SMINT1000051 | 0 | 0 | 0 | 0 | 100 | 0 | 0 |
| SMINT1000054 | 0 | 0 | 0 | 0 | 66.007 | 0 | 0 |
| SMINT1000057 | 0 | 0 | 0 | 0 | 62.065 | 0 | 0 |
| SMINT1000071 | 0 | 0 | 0 | 0 | 100 | 0 | 0 |
| SMINT1000075 | 0 | 0 | 0 | 0 | 100 | 0 | 0 |
| SMINT1000078 | 0 | 0 | 0 | 0 | 33.223 | 0 | 0 |
| SMINT1000091 | 0 | 0 | 0 | 0 | 100 | 0 | 0 |
| SMINT1000100 | 0 | 0 | 0 | 0 | 19.647 | 0 | 0 |
| SMINT1000103 | 0 | 0 | 0 | 0 | 100 | 0 | 0 |
| SMINT1000117 | 0 | 0 | 0 | 0 | 4.641 | 0 | 0 |
| SMINT1000118 | 0 | 0 | 0 | 0 | 100 | 0 | 0 |
| SMINT1000131 | 0 | 0 | 0 | 0 | 3.232 | 0 | 6.232 |
| SMINT1000137 | 0 | 0 | 0 | 0 | 66.980 | 33.020 | 0 |
| SMINT1000192 | 0 | 0 | 0 | 0 | 10.508 | 0 | 0 |
| SMINT2000007 | 0 | 0 | 0 | 0 | 42.450 | 10.464 | 0 |
| SMINT2000018 | 0 | 0 | 0 | 0 | 14.856 | 0 | 0 |
| SMINT2000032 | 0 | 0 | 0 | 0 | 100 | 0 | 0 |
| SMINT2000040 | 0 | 0 | 0 | 0 | 100 | 0 | 0 |
| SMINT2000060 | 0 | 0 | 0 | 0 | 100 | 0 | 0 |
| SMINT2000073 | 0 | 0 | 0 | 0 | 30.613 | 0 | 0 |
| SMINT2000084 | 0 | 0 | 0 | 0 | 100 | 0 | 0 |
| SMINT2000145 | 0 | 0 | 0 | 0 | 100 | 0 | 0 |
| SMINT2000159 | 0 | 0 | 0 | 0 | 38.570 | 9.507 | 0 |
| SMINT2000176 | 0 | 0 | 0 | 0 | 49.333 | 0 | 0 |
| SMINT2000185 | 0 | 0 | 0 | 0 | 100 | 0 | 0 |
| SMINT2000227 | 0 | 0 | 0 | 0 | 100 | 0 | 0 |
| SMINT2000229 | 0 | 0 | 0 | 0 | 100 | 0 | 0 |
| SMINT2000232 | 0 | 0 | 0 | 0 | 50.236 | 0 | 0 |
| SMINT2000239 | 0 | 0 | 0 | 0 | 29.598 | 0 | 0 |
| SMINT2000267 | 0 | 0 | 0 | 0 | 76.088 | 0 | 0 |
| SMINT2000277 | 0 | 0 | 0 | 0 | 6.397 | 3.154 | 0 |
| SMINT2000396 | 0 | 0 | 0 | 0 | 4.320 | 2.130 | 0 |
| SMINT2000400 | 0 | 0 | 0 | 0 | 23.606 | 0 | 0 |
| SMINT2000441 | 0 | 0 | 0 | 0 | 1.593 | 1.570 | 0 |
| SMINT2000454 | 0 | 0 | 0 | 0 | 31.537 | 0 | 0 |
| SMINT2000468 | 0 | 0 | 0 | 0 | 8.074 | 0 | 0 |
| SMINT2000518 | 0 | 0 | 0 | 0 | 100 | 0 | 0 |
| SMINT2000529 | 0 | 0 | 0 | 0 | 100 | 0 | 0 |
| SMINT2000530 | 0 | 0 | 0 | 0 | 100 | 0 | 0 |
| SMINT2000537 | 0 | 0 | 0 | 0 | 73.962 | 0 | 0 |
| SMINT2000538 | 0 | 0 | 0 | 0 | 100 | 0 | 0 |
| SMINT2000541 | 0 | 0 | 0 | 0 | 100 | 0 | 0 |
| SMINT2000545 | 0 | 0 | 0 | 0 | 8.365 | 8.248 | 0 |
| SMINT2000558 | 0 | 0 | 0 | 0 | 100 | 0 | 0 |
| SMINT2000562 | 0 | 0 | 0 | 0 | 100 | 0 | 0 |
| SMINT2000568 | 0 | 0 | 0 | 0 | 100 | 0 | 0 |
| SMINT2000602 | 0 | 0 | 0 | 0 | 1.940 | 2.869 | 0 |
| SMINT2000609 | 0 | 0 | 0 | 0 | 66.007 | 0 | 0 |
| SMINT2000629 | 0 | 0 | 0 | 0 | 9.477 | 0 | 0 |
| SMINT2000659 | 0 | 0 | 0 | 0 | 1.961 | 0 | 0 |
| SMINT2000694 | 0 | 0 | 0 | 0 | 4.194 | 0 | 0 |
| SMINT2000747 | 0 | 0 | 0 | 0 | 57.827 | 28.508 | 0 |
| SMINT2000811 | 0 | 0 | 0 | 0 | 100 | 0 | 0 |
| SMINT2000824 | 0 | 0 | 0 | 0 | 100 | 0 | 0 |
| SMINT2000933 | 0 | 0 | 0 | 0 | 3.596 | 10.638 | 0 |
| SMINT2000984 | 0 | 0 | 0 | 0 | 100 | 0 | 0 |
| SMINT2001114 | 0 | 0 | 0 | 0 | 100 | 0 | 0 |
| SMINT2001158 | 0 | 0 | 0 | 0 | 100 | 0 | 0 |
| SMINT2001183 | 0 | 0 | 0 | 0 | 32.042 | 0 | 0 |
| SMINT2001199 | 0 | 0 | 0 | 0 | 35.321 | 0 | 0 |
| SMINT2001222 | 0 | 0 | 0 | 0 | 14.585 | 0 | 0 |
| SMINT2001348 | 0 | 0 | 0 | 0 | 32.736 | 0 | 0 |
| SMINT2001397 | 0 | 0 | 0 | 0 | 100 | 0 | 0 |
| SMINT2001458 | 0 | 0 | 0 | 0 | 100 | 0 | 0 |
| SMINT2001461 | 0 | 0 | 0 | 0 | 100 | 0 | 0 |
| SMINT2001535 | 0 | 0 | 0 | 0 | 100 | 0 | 0 |
| SMINT2001559 | 0 | 0 | 0 | 0 | 100 | 0 | 0 |
| SMINT2001609 | 0 | 0 | 0 | 0 | 100 | 0 | 0 |
| SMINT2001615 | 0 | 0 | 0 | 0 | 59.418 | 0 | 0 |
| SMINT2001683 | 0 | 0 | 0 | 0 | 100 | 0 | 0 |
| SMINT2001731 | 0 | 0 | 0 | 0 | 100 | 0 | 0 |
| SMINT2001812 | 0 | 0 | 0 | 0 | 56.438 | 0 | 0 |
| SMINT2001818 | 0 | 0 | 0 | 0 | 9.924 | 3.261 | 12.758 |
| SMINT2001950 | 0 | 0 | 0 | 0 | 75.408 | 0 | 0 |
| SMINT2002126 | 0 | 0 | 0 | 0 | 100 | 0 | 0 |
| SMINT2002159 | 0 | 0 | 0 | 0 | 24.194 | 0 | 0 |
| SMINT2002202 | 0 | 0 | 0 | 0 | 100 | 0 | 0 |
| SMINT2002210 | 0 | 0 | 0 | 0 | 100 | 0 | 0 |
| SMINT2002224 | 0 | 0 | 0 | 0 | 44.673 | 0 | 0 |
| SMINT2002281 | 0 | 0 | 0 | 0 | 100 | 0 | 0 |
| SMINT2002311 | 0 | 0 | 0 | 0 | 18.692 | 9.215 | 72.094 |
| SMINT2002314 | 0 | 0 | 0 | 0 | 4.271 | 0 | 0 |
| SMINT2002328 | 0 | 0 | 0 | 0 | 65.453 | 0 | 0 |
| SMINT2002414 | 0 | 0 | 0 | 0 | 100 | 0 | 0 |
| SMINT2002457 | 0 | 0 | 0 | 0 | 100 | 0 | 0 |
| SMINT2002620 | 0 | 0 | 0 | 0 | 7.995 | 0 | 0 |
| SMINT2002689 | 0 | 0 | 0 | 0 | 4.148 | 0 | 0 |
| SMINT2002743 | 0 | 0 | 0 | 0 | 100 | 0 | 0 |
| SMINT2002778 | 0 | 0 | 0 | 0 | 74.739 | 0 | 0 |
| SMINT2002880 | 0 | 0 | 0 | 0 | 100 | 0 | 0 |
| SMINT2002882 | 0 | 0 | 0 | 0 | 10.246 | 0 | 0 |
| SMINT2002884 | 0 | 0 | 0 | 0 | 13.034 | 6.426 | 0 |
| SMINT2002899 | 0 | 0 | 0 | 0 | 100 | 0 | 0 |
| SMINT2002974 | 0 | 0 | 0 | 0 | 100 | 0 | 0 |
| SMINT2002976 | 0 | 0 | 0 | 0 | 18.406 | 0 | 0 |
| SMINT2003003 | 0 | 0 | 0 | 0 | 69.568 | 0 | 0 |
| SMINT2003074 | 0 | 0 | 0 | 0 | 4.837 | 0 | 9.328 |
| SMINT2003128 | 0 | 0 | 0 | 0 | 100 | 0 | 0 |
| SMINT2003150 | 0 | 0 | 0 | 0 | 77.788 | 0 | 0 |
| SMINT2003169 | 0 | 0 | 0 | 3.230 | 1.660 | 2.455 | 0 |
| SMINT2003317 | 0 | 0 | 0 | 0 | 100 | 0 | 0 |
| SMINT2003340 | 0 | 0 | 0 | 0 | 10.368 | 5.111 | 0 |
| SMINT2003386 | 0 | 0 | 0 | 0 | 100 | 0 | 0 |
| SMINT2003423 | 0 | 0 | 0 | 0 | 38.336 | 0 | 0 |
| SMINT2003505 | 0 | 0 | 0 | 0 | 11.112 | 5.478 | 0 |
| SMINT2003551 | 0 | 0 | 0 | 0 | 48.952 | 0 | 0 |
| SMINT2003569 | 0 | 0 | 0 | 0 | 100 | 0 | 0 |
| SMINT2003641 | 0 | 0 | 0 | 0 | 100 | 0 | 0 |
| SMINT2003644 | 0 | 0 | 0 | 0 | 100 | 0 | 0 |
| SMINT2003866 | 0 | 0 | 0 | 0 | 100 | 0 | 0 |
| SMINT2003905 | 0 | 0 | 0 | 0 | 2.732 | 0 | 0 |
| SMINT2004023 | 0 | 10.746 | 0 | 0 | 5.382 | 0 | 0 |
| SMINT2004037 | 0 | 0 | 0 | 0 | 67.907 | 0 | 0 |
| SMINT2004047 | 0 | 0 | 0 | 0 | 100 | 0 | 0 |
| SMINT2004086 | 0 | 0 | 0 | 0 | 100 | 0 | 0 |
| SMINT2004144 | 0 | 0 | 0 | 0 | 4.805 | 0 | 0 |
| SMINT2004280 | 0 | 0 | 0 | 0 | 44.959 | 0 | 0 |
| SMINT2004299 | 0 | 0 | 0 | 0 | 31.847 | 0 | 0 |
| SMINT2004339 | 0 | 0 | 0 | 0 | 100 | 0 | 0 |
| SMINT2004350 | 0 | 0 | 0 | 0 | 46.110 | 0 | 0 |
| SMINT2004414 | 0 | 0 | 0 | 0 | 83.254 | 0 | 0 |
| SMINT2004473 | 0 | 0 | 0 | 0 | 100 | 0 | 0 |
| SMINT2004547 | 0 | 0 | 0 | 0 | 100 | 0 | 0 |
| SMINT2004583 | 0 | 0 | 0 | 0 | 13.670 | 0 | 0 |
| SMINT2004589 | 0 | 0 | 0 | 0 | 28.285 | 0 | 27.274 |
| SMINT2004729 | 0 | 0 | 0 | 0 | 45.320 | 0 | 0 |
| SMINT2004781 | 0 | 0 | 0 | 0 | 100 | 0 | 0 |
| SMINT2004872 | 0 | 0 | 0 | 0 | 14.926 | 0 | 0 |
| SMINT2004891 | 0 | 0 | 0 | 0 | 100 | 0 | 0 |
| SMINT2004909 | 0 | 0 | 0 | 0 | 100 | 0 | 0 |
| SMINT2004972 | 0 | 0 | 0 | 0 | 27.951 | 0 | 0 |
| SMINT2004992 | 0 | 0 | 0 | 0 | 100 | 0 | 0 |
| SMINT2005075 | 0 | 0 | 0 | 0 | 100 | 0 | 0 |
| SMINT2005161 | 0 | 0 | 0 | 0 | 100 | 0 | 0 |
| SMINT2005174 | 0 | 0 | 0 | 0 | 100 | 0 | 0 |
| SMINT2005213 | 0 | 0 | 0 | 0 | 100 | 0 | 0 |
| SMINT2005330 | 0 | 0 | 0 | 0 | 48.668 | 0 | 0 |
| SMINT2005368 | 0 | 0 | 0 | 0 | 12.432 | 0 | 0 |
| SMINT2005387 | 0 | 0 | 0 | 0 | 80.886 | 0 | 0 |
| SMINT2005402 | 0 | 0 | 0 | 0 | 83.254 | 0 | 0 |
| SMINT2005405 | 0 | 0 | 0 | 0 | 100 | 0 | 0 |
| SMINT2005621 | 0 | 0 | 0 | 0 | 100 | 0 | 0 |
| SMINT2005623 | 0 | 0 | 0 | 0 | 100 | 0 | 0 |
| SMINT2005624 | 0 | 0 | 0 | 0 | 100 | 0 | 0 |
| SMINT2005688 | 0 | 0 | 0 | 0 | 8.807 | 0 | 0 |
| SMINT2005800 | 0 | 0 | 0 | 0 | 100 | 0 | 0 |
| SMINT2005956 | 0 | 18.546 | 0 | 0 | 9.288 | 0 | 0 |
| SMINT2006078 | 0 | 0 | 0 | 0 | 100 | 0 | 0 |
| SMINT2006205 | 0 | 0 | 0 | 0 | 100 | 0 | 0 |
| SMINT2006331 | 0 | 0 | 0 | 0 | 100 | 0 | 0 |
| SMINT2006596 | 0 | 0 | 0 | 0 | 100 | 0 | 0 |
| SMINT2006641 | 0 | 0 | 0 | 0 | 100 | 0 | 0 |
| SMINT2006648 | 0 | 0 | 0 | 0 | 100 | 0 | 0 |
| SMINT2006708 | 0 | 0 | 0 | 0 | 100 | 0 | 0 |
| SMINT2006716 | 0 | 0 | 0 | 0 | 41.444 | 0 | 0 |
| SMINT2006801 | 0 | 0 | 0 | 0 | 56.833 | 0 | 0 |
| SMINT2007062 | 0 | 0 | 0 | 0 | 80.886 | 0 | 0 |
| SMINT2007101 | 0 | 0 | 0 | 0 | 76.088 | 0 | 0 |
| SMINT2007140 | 0 | 0 | 0 | 0 | 100 | 0 | 0 |
| SMINT2007187 | 0 | 0 | 0 | 0 | 23.689 | 0 | 0 |
| SMINT2007219 | 0 | 0 | 0 | 0 | 100 | 0 | 0 |
| SMINT2007294 | 0 | 0 | 0 | 0 | 100 | 0 | 0 |
| SMINT2007365 | 0 | 0 | 0 | 0 | 100 | 0 | 0 |
| SMINT2007391 | 0 | 0 | 0 | 0 | 100 | 0 | 0 |
| SMINT2007433 | 0 | 0 | 0 | 0 | 29.456 | 0 | 0 |
| SMINT2007508 | 0 | 0 | 0 | 0 | 58.601 | 0 | 0 |
| SMINT2007526 | 0 | 0 | 0 | 0 | 100 | 0 | 0 |
| SMINT2007579 | 0 | 0 | 0 | 0 | 100 | 0 | 0 |
| SMINT2007647 | 0 | 0 | 0 | 0 | 77.788 | 0 | 0 |
| SMINT2007729 | 0 | 0 | 0 | 0 | 20.292 | 0 | 0 |
| SMINT2007790 | 0 | 0 | 0 | 0 | 100 | 0 | 0 |
| SMINT2007792 | 0 | 0 | 0 | 0 | 29.494 | 0 | 0 |
| SMINT2007796 | 0 | 0 | 0 | 0 | 100 | 0 | 0 |
| SMINT2007867 | 0 | 0 | 0 | 0 | 100 | 0 | 0 |
| SMINT2007932 | 0 | 0 | 0 | 0 | 100 | 0 | 0 |
| SMINT2008042 | 0 | 0 | 0 | 0 | 100 | 0 | 0 |
| SMINT2008054 | 0 | 0 | 0 | 0 | 66.980 | 33.020 | 0 |
| SMINT2008133 | 0 | 0 | 0 | 0 | 83.254 | 0 | 0 |
| SMINT2008277 | 0 | 0 | 0 | 0 | 100 | 0 | 0 |
| SMINT2008329 | 0 | 0 | 0 | 0 | 100 | 0 | 0 |
| SMINT2008455 | 0 | 0 | 0 | 0 | 100 | 0 | 0 |
| SMINT2008491 | 0 | 0 | 0 | 0 | 100 | 0 | 0 |
| SMINT2008531 | 0 | 0 | 0 | 0 | 100 | 0 | 0 |
| SMINT2008545 | 0 | 0 | 0 | 0 | 100 | 0 | 0 |
| SMINT2008558 | 0 | 0 | 0 | 0 | 100 | 0 | 0 |
| SMINT2008590 | 0 | 0 | 0 | 0 | 33.779 | 0 | 0 |
| SMINT2008625 | 0 | 0 | 0 | 0 | 100 | 0 | 0 |
| SMINT2008672 | 0 | 0 | 0 | 0 | 47.955 | 0 | 0 |
| SMINT2008690 | 0 | 0 | 0 | 0 | 100 | 0 | 0 |
| SMINT2008715 | 0 | 0 | 0 | 0 | 100 | 0 | 0 |
| SMINT2008720 | 0 | 0 | 0 | 0 | 100 | 0 | 0 |
| SMINT2008817 | 0 | 0 | 0 | 0 | 100 | 0 | 0 |
| SMINT2008844 | 0 | 0 | 0 | 0 | 100 | 0 | 0 |
| SMINT2008869 | 0 | 0 | 0 | 0 | 49.902 | 0 | 0 |
| SMINT2008917 | 0 | 0 | 0 | 0 | 39.482 | 0 | 0 |
| SMINT2008921 | 0 | 0 | 0 | 0 | 3.915 | 0 | 0 |
| SMINT2008960 | 0 | 0 | 0 | 0 | 2.414 | 2.380 | 4.656 |
| SMINT2009119 | 0 | 0 | 0 | 0 | 100 | 0 | 0 |
| SMINT2009121 | 0 | 0 | 0 | 0 | 100 | 0 | 0 |
| SMINT2009212 | 0 | 0 | 0 | 0 | 100 | 0 | 0 |
| SMINT2009216 | 0 | 0 | 0 | 0 | 19.533 | 0 | 0 |
| SMINT2009233 | 0 | 0 | 0 | 0 | 83.254 | 0 | 0 |
| SMINT2009259 | 0 | 0 | 0 | 0 | 100 | 0 | 0 |
| SMINT2009272 | 0 | 0 | 0 | 0 | 100 | 0 | 0 |
| SMINT2009292 | 0 | 0 | 0 | 0 | 12.516 | 0 | 0 |
| SMINT2009363 | 0 | 0 | 0 | 0 | 100 | 0 | 0 |
| SMINT2009415 | 0 | 0 | 0 | 0 | 76.088 | 0 | 0 |
| SMINT2009468 | 0 | 0 | 0 | 0 | 100 | 0 | 0 |
| SMINT2009505 | 0 | 0 | 0 | 0 | 100 | 0 | 0 |
| SMINT2009529 | 0 | 0 | 0 | 0 | 100 | 0 | 0 |
| SMINT2009832 | 0 | 0 | 0 | 0 | 32.604 | 0 | 0 |
| SMINT2009895 | 0 | 0 | 0 | 0 | 100 | 0 | 0 |
| SMINT2009902 | 0 | 0 | 0 | 0 | 100 | 0 | 0 |
| SMINT2009973 | 0 | 0 | 0 | 0 | 100 | 0 | 0 |
| SMINT2010015 | 0 | 0 | 0 | 0 | 100 | 0 | 0 |
| SMINT2010068 | 0 | 0 | 0 | 0 | 100 | 0 | 0 |
| SMINT2010076 | 0 | 0 | 0 | 0 | 100 | 0 | 0 |
| SMINT2010084 | 0 | 0 | 0 | 0 | 12.125 | 5.977 | 0 |
| SMINT2010144 | 0 | 0 | 0 | 0 | 76.088 | 0 | 0 |
| SMINT2010200 | 0 | 0 | 0 | 0 | 33.450 | 0 | 0 |
| SMINT2010278 | 0 | 0 | 0 | 0 | 100 | 0 | 0 |
| SMINT2010324 | 0 | 0 | 0 | 0 | 76.088 | 0 | 0 |
| SMINT2010326 | 0 | 0 | 0 | 0 | 100 | 0 | 0 |
| SMINT2010369 | 0 | 0 | 0 | 0 | 80.886 | 0 | 0 |
| SMINT2010426 | 0 | 0 | 42.229 | 0 | 19.116 | 0 | 0 |
| SMINT2010500 | 0 | 0 | 0 | 0 | 100 | 0 | 0 |
| SMINT2010533 | 0 | 0 | 0 | 0 | 100 | 0 | 0 |
| SMINT2010629 | 0 | 0 | 0 | 0 | 100 | 0 | 0 |
| SMINT2010639 | 0 | 0 | 0 | 0 | 9.559 | 0 | 0 |
| SMINT2010672 | 0 | 0 | 0 | 0 | 100 | 0 | 0 |
| SMINT2010753 | 0 | 0 | 0 | 0 | 100 | 0 | 0 |
| SMINT2010853 | 0 | 0 | 0 | 0 | 100 | 0 | 0 |
| SMINT2010897 | 0 | 0 | 0 | 0 | 100 | 0 | 0 |
| SMINT2010901 | 0 | 0 | 0 | 0 | 12.393 | 6.110 | 0 |
| SMINT2010959 | 0 | 0 | 0 | 0 | 54.616 | 26.925 | 0 |
| SMINT2010997 | 0 | 0 | 0 | 0 | 100 | 0 | 0 |
| SMINT2011033 | 0 | 0 | 0 | 0 | 74.739 | 0 | 0 |
| SMINT2011066 | 0 | 0 | 0 | 0 | 100 | 0 | 0 |
| SMINT2011273 | 0 | 0 | 0 | 0 | 100 | 0 | 0 |
| SMINT2011311 | 0 | 0 | 0 | 0 | 66.474 | 0 | 0 |
| SMINT2011406 | 0 | 0 | 0 | 0 | 100 | 0 | 0 |
| SMINT2011509 | 0 | 0 | 0 | 0 | 100 | 0 | 0 |
| SMINT2011567 | 0 | 0 | 0 | 0 | 33.480 | 0 | 0 |
| SMINT2011588 | 0 | 0 | 0 | 0 | 65.637 | 0 | 0 |
| SMINT2011656 | 0 | 0 | 0 | 0 | 83.254 | 0 | 0 |
| SMINT2011888 | 0 | 0 | 0 | 0 | 100 | 0 | 0 |
| SMINT2011958 | 0 | 0 | 0 | 0 | 100 | 0 | 0 |
| SMINT2012040 | 0 | 0 | 0 | 0 | 100 | 0 | 0 |
| SMINT2012122 | 0 | 0 | 0 | 0 | 12.336 | 0 | 0 |
| SMINT2012179 | 0 | 0 | 0 | 0 | 65.556 | 0 | 0 |
| SMINT2012195 | 0 | 0 | 0 | 0 | 83.254 | 0 | 0 |
| SMINT2012285 | 0 | 0 | 0 | 0 | 100 | 0 | 0 |
| SMINT2012291 | 0 | 0 | 0 | 0 | 16.272 | 0 | 0 |
| SMINT2012501 | 0 | 0 | 0 | 0 | 28.061 | 0 | 0 |
| SMINT2012734 | 0 | 0 | 0 | 0 | 100 | 0 | 0 |
| SMINT2012793 | 0 | 0 | 0 | 0 | 100 | 0 | 0 |
| SMINT2013031 | 0 | 0 | 0 | 0 | 10.952 | 0 | 0 |
| SMINT2013032 | 0 | 0 | 0 | 0 | 42.206 | 0 | 0 |
| SMINT2013129 | 0 | 0 | 0 | 0 | 100 | 0 | 0 |
| SMINT2013170 | 0 | 0 | 0 | 0 | 38.476 | 0 | 0 |
| SMINT2013181 | 0 | 0 | 0 | 0 | 77.788 | 0 | 0 |
| SMINT2013228 | 0 | 0 | 0 | 0 | 100 | 0 | 0 |
| SMINT2013613 | 0 | 0 | 0 | 0 | 100 | 0 | 0 |
| SMINT2013626 | 0 | 0 | 0 | 0 | 100 | 0 | 0 |
| SMINT2013695 | 0 | 0 | 0 | 0 | 74.739 | 0 | 0 |
| SMINT2013833 | 0 | 0 | 0 | 0 | 100 | 0 | 0 |
| SMINT2013890 | 0 | 0 | 0 | 0 | 32.378 | 0 | 0 |
| SMINT2014166 | 0 | 0 | 0 | 0 | 100 | 0 | 0 |
| SMINT2014282 | 0 | 0 | 0 | 0 | 100 | 0 | 0 |
| SMINT2014443 | 0 | 0 | 0 | 0 | 100 | 0 | 0 |
| SMINT2014480 | 0 | 0 | 0 | 0 | 5.606 | 0 | 0 |
| SMINT2014489 | 0 | 0 | 0 | 0 | 100 | 0 | 0 |
| SMINT2014721 | 0 | 0 | 0 | 0 | 100 | 0 | 0 |
| SMINT2015006 | 0 | 0 | 0 | 0 | 100 | 0 | 0 |
| SMINT2015294 | 0 | 0 | 0 | 0 | 28.094 | 0 | 0 |
| SMINT2015306 | 0 | 0 | 0 | 0 | 35.321 | 0 | 0 |
| SMINT2015326 | 0 | 0 | 0 | 0 | 100 | 0 | 0 |
| SMINT2015353 | 0 | 0 | 0 | 0 | 100 | 0 | 0 |
| SMINT2015454 | 0 | 0 | 0 | 0 | 100 | 0 | 0 |
| SMINT2015518 | 0 | 0 | 0 | 0 | 100 | 0 | 0 |
| SMINT2015745 | 0 | 0 | 0 | 0 | 100 | 0 | 0 |
| SMINT2015787 | 0 | 0 | 0 | 0 | 100 | 0 | 0 |
| SMINT2015810 | 0 | 0 | 0 | 0 | 100 | 0 | 0 |
| SMINT2016122 | 0 | 0 | 0 | 0 | 100 | 0 | 0 |
| SMINT2016146 | 0 | 0 | 0 | 0 | 60.601 | 0 | 0 |
| SMINT2016286 | 0 | 0 | 0 | 0 | 100 | 0 | 0 |
| SMINT2016313 | 0 | 0 | 0 | 0 | 100 | 0 | 0 |
| SMINT2016396 | 0 | 0 | 0 | 0 | 49.902 | 0 | 0 |
| SMINT2016412 | 0 | 0 | 0 | 0 | 19.978 | 0 | 0 |
| SMINT2016440 | 0 | 0 | 0 | 0 | 100 | 0 | 0 |
| SMINT2016477 | 0 | 0 | 0 | 0 | 100 | 0 | 0 |
| SMINT2016544 | 0 | 0 | 0 | 0 | 100 | 0 | 0 |
| SMINT2016857 | 0 | 0 | 0 | 0 | 100 | 0 | 0 |
| SMINT2017134 | 0 | 0 | 0 | 0 | 76.088 | 0 | 0 |
| SMINT2017319 | 0 | 0 | 0 | 0 | 100 | 0 | 0 |
| SMINT2017324 | 0 | 0 | 0 | 0 | 100 | 0 | 0 |
| SMINT2017410 | 0 | 0 | 0 | 0 | 100 | 0 | 0 |
| SMINT2017431 | 0 | 0 | 0 | 0 | 100 | 0 | 0 |
| SMINT2017432 | 0 | 0 | 0 | 0 | 100 | 0 | 0 |
| SMINT2017436 | 0 | 0 | 0 | 0 | 100 | 0 | 0 |
| SMINT2017599 | 0 | 0 | 0 | 0 | 14.490 | 0 | 0 |
| SMINT2017659 | 0 | 0 | 0 | 0 | 100 | 0 | 0 |
| SMINT2017736 | 0 | 0 | 0 | 0 | 100 | 0 | 0 |
| SMINT2017781 | 0 | 0 | 0 | 0 | 100 | 0 | 0 |
| SMINT2017855 | 0 | 0 | 0 | 0 | 100 | 0 | 0 |
| SMINT2017884 | 0 | 0 | 0 | 0 | 83.254 | 0 | 0 |
| SMINT2017964 | 0 | 0 | 0 | 0 | 17.105 | 0 | 0 |
| SMINT2017974 | 0 | 0 | 0 | 0 | 100 | 0 | 0 |
| SMINT2018343 | 0 | 0 | 0 | 0 | 100 | 0 | 0 |
| SMINT2018353 | 0 | 0 | 0 | 0 | 34.595 | 0 | 0 |
| SMINT2018592 | 0 | 0 | 0 | 0 | 100 | 0 | 0 |
| SMINT2018681 | 0 | 0 | 0 | 0 | 14.757 | 0 | 0 |
| SMINT2019017 | 0 | 0 | 0 | 0 | 100 | 0 | 0 |
| SMINT2019105 | 0 | 0 | 0 | 0 | 100 | 0 | 0 |
| SMINT2019142 | 0 | 0 | 0 | 0 | 100 | 0 | 0 |
| SMINT2019153 | 0 | 0 | 0 | 0 | 100 | 0 | 0 |
| SMINT2019200 | 0 | 0 | 0 | 0 | 100 | 0 | 0 |
| SPLEN1000013 | 0 | 0 | 0 | 0 | 0 | 16.928 | 0 |
| SPLEN1000041 | 0 | 0 | 0 | 0 | 0 | 67.599 | 0 |
| SPLEN1000049 | 0 | 0 | 0 | 0 | 0 | 37.925 | 0 |
| SPLEN1000056 | 0 | 0 | 0 | 0 | 0 | 67.599 | 0 |
| SPLEN1000087 | 0 | 0 | 0 | 0 | 0 | 100 | 0 |
| SPLEN1000089 | 0 | 0 | 0 | 0 | 0 | 6.913 | 0 |
| SPLEN1000091 | 0 | 0 | 0 | 0 | 0 | 100 | 0 |
| SPLEN1000106 | 0 | 0 | 0 | 0 | 0 | 8.098 | 0 |
| SPLEN1000116 | 0 | 0 | 0 | 0 | 0 | 4.305 | 0 |
| SPLEN1000134 | 0 | 0 | 0 | 0 | 0 | 60.831 | 0 |
| SPLEN1000139 | 0 | 0 | 0 | 0 | 0 | 49.431 | 0 |
| SPLEN1000143 | 0 | 0 | 0 | 0 | 0 | 100 | 0 |
| SPLEN1000144 | 0 | 0 | 0 | 0 | 0 | 18.188 | 0 |
| SPLEN1000145 | 0 | 0 | 0 | 0 | 0 | 100 | 0 |
| SPLEN1000156 | 0 | 0 | 0 | 0 | 0 | 100 | 0 |
| SPLEN1000163 | 0 | 0 | 0 | 0 | 0 | 100 | 0 |
| SPLEN1000165 | 0 | 0 | 0 | 0 | 0 | 8.681 | 0 |
| SPLEN1000166 | 0 | 0 | 0 | 0 | 0 | 1.967 | 7.693 |
| SPLEN1000178 | 0 | 0 | 0 | 0 | 0 | 100 | 0 |
| SPLEN2000020 | 0 | 0 | 0 | 0 | 0 | 100 | 0 |
| SPLEN2000047 | 0 | 0 | 0 | 0 | 0 | 28.708 | 0 |
| SPLEN2000064 | 0 | 0 | 0 | 0 | 0 | 100 | 0 |
| SPLEN2000072 | 0 | 0 | 0 | 0 | 42.383 | 10.447 | 0 |
| SPLEN2000098 | 0 | 0 | 0 | 0 | 0 | 20.954 | 0 |
| SPLEN2000126 | 0 | 0 | 0 | 0 | 0 | 100 | 0 |
| SPLEN2000134 | 0 | 0 | 0 | 0 | 0 | 9.114 | 0 |
| SPLEN2000143 | 0 | 0 | 0 | 0 | 0 | 80.667 | 0 |
| SPLEN2000189 | 0 | 0 | 0 | 0 | 0 | 100 | 0 |
| SPLEN2000197 | 0 | 0 | 0 | 0 | 0 | 61.070 | 0 |
| SPLEN2000205 | 0 | 0 | 0 | 0 | 0 | 100 | 0 |
| SPLEN2000210 | 0 | 0 | 0 | 0 | 0 | 23.091 | 0 |
| SPLEN2000222 | 0 | 0 | 0 | 0 | 0 | 21.249 | 0 |
| SPLEN2000242 | 0 | 0 | 0 | 0 | 0 | 16.714 | 0 |
| SPLEN2000243 | 0 | 0 | 0 | 0 | 0 | 37.818 | 0 |
| SPLEN2000247 | 0 | 0 | 0 | 0 | 0 | 100 | 0 |
| SPLEN2000255 | 0 | 0 | 0 | 0 | 21.677 | 18.702 | 0 |
| SPLEN2000258 | 0 | 0 | 0 | 0 | 15.448 | 7.616 | 0 |
| SPLEN2000267 | 0 | 0 | 0 | 0 | 0 | 10.156 | 0 |
| SPLEN2000270 | 0 | 0 | 0 | 0 | 0 | 100 | 0 |
| SPLEN2000307 | 0 | 0 | 0 | 0 | 0 | 100 | 0 |
| SPLEN2000310 | 0 | 0 | 0 | 0 | 0 | 23.091 | 0 |
| SPLEN2000341 | 0 | 0 | 0 | 0 | 47.783 | 23.557 | 0 |
| SPLEN2000348 | 0 | 0 | 0 | 0 | 0 | 9.733 | 0 |
| SPLEN2000350 | 0 | 0 | 0 | 0 | 0 | 100 | 0 |
| SPLEN2000357 | 0 | 0 | 0 | 0 | 0 | 100 | 0 |
| SPLEN2000364 | 0 | 0 | 0 | 0 | 0 | 100 | 0 |
| SPLEN2000402 | 0 | 0 | 0 | 0 | 0 | 100 | 0 |
| SPLEN2000414 | 0 | 0 | 0 | 0 | 0 | 23.091 | 0 |
| SPLEN2000420 | 0 | 0 | 0 | 0 | 0 | 100 | 0 |
| SPLEN2000443 | 0 | 0 | 0 | 0 | 0 | 38.429 | 0 |
| SPLEN2000448 | 0 | 0 | 0 | 0 | 0 | 100 | 0 |
| SPLEN2000450 | 0 | 0 | 0 | 0 | 0 | 100 | 0 |
| SPLEN2000464 | 0 | 0 | 0 | 0 | 0 | 100 | 0 |
| SPLEN2000496 | 0 | 0 | 0 | 0 | 0 | 100 | 0 |
| SPLEN2000505 | 0 | 0 | 3.285 | 0 | 0 | 0.733 | 0 |
| SPLEN2000515 | 0 | 0 | 0 | 0 | 0 | 100 | 0 |
| SPLEN2000516 | 0 | 0 | 0 | 0 | 0 | 41.589 | 0 |
| SPLEN2000537 | 0 | 0 | 0 | 0 | 0 | 63.499 | 0 |
| SPLEN2000541 | 0 | 0 | 0 | 0 | 0 | 100 | 0 |
| SPLEN2000607 | 0 | 0 | 0 | 0 | 0 | 100 | 0 |
| SPLEN2000630 | 0 | 0 | 0 | 0 | 0 | 100 | 0 |
| SPLEN2000695 | 0 | 0 | 0 | 0 | 0 | 100 | 0 |
| SPLEN2000698 | 0 | 0 | 0 | 0 | 0 | 3.689 | 0 |
| SPLEN2000839 | 0 | 0 | 0 | 0 | 0 | 15.878 | 0 |
| SPLEN2000874 | 0 | 0 | 0 | 3.347 | 0 | 0.848 | 0 |
| SPLEN2000882 | 0 | 0 | 0 | 0 | 6.199 | 12.224 | 0 |
| SPLEN2001135 | 0 | 0 | 0 | 0 | 0 | 100 | 0 |
| SPLEN2001141 | 0 | 0 | 0 | 0 | 0 | 8.214 | 0 |
| SPLEN2001157 | 0 | 0 | 42.883 | 0 | 0 | 9.570 | 0 |
| SPLEN2001176 | 0 | 0 | 0 | 0 | 0 | 100 | 0 |
| SPLEN2001227 | 0 | 0 | 0 | 0 | 0 | 100 | 0 |
| SPLEN2001245 | 0 | 0 | 0 | 0 | 0 | 1.944 | 0 |
| SPLEN2001278 | 0 | 0 | 0 | 0 | 0 | 67.599 | 0 |
| SPLEN2001354 | 0 | 0 | 0 | 0 | 0 | 100 | 0 |
| SPLEN2001367 | 0 | 0 | 0 | 0 | 0 | 100 | 0 |
| SPLEN2001383 | 0 | 0 | 0 | 0 | 0 | 100 | 0 |
| SPLEN2001476 | 0 | 0 | 0 | 0 | 0 | 67.599 | 0 |
| SPLEN2001503 | 0 | 0 | 0 | 0 | 0 | 34.999 | 0 |
| SPLEN2001510 | 0 | 0 | 0 | 0 | 0 | 100 | 0 |
| SPLEN2001599 | 0 | 0 | 0 | 0 | 0 | 24.827 | 0 |
| SPLEN2001626 | 0 | 0 | 0 | 0 | 0 | 32.545 | 0 |
| SPLEN2001650 | 0 | 0 | 0 | 0 | 0 | 100 | 0 |
| SPLEN2001689 | 0 | 0 | 0 | 0 | 0 | 48.561 | 0 |
| SPLEN2001710 | 0 | 0 | 0 | 0 | 0 | 13.491 | 0 |
| SPLEN2001761 | 0 | 0 | 0 | 0 | 0 | 18.511 | 0 |
| SPLEN2001781 | 0 | 0 | 0 | 0 | 0 | 100 | 0 |
| SPLEN2001881 | 0 | 0 | 0 | 0 | 0 | 100 | 0 |
| SPLEN2001912 | 0 | 0 | 0 | 0 | 0 | 100 | 0 |
| SPLEN2001945 | 0 | 0 | 0 | 0 | 0 | 100 | 0 |
| SPLEN2002007 | 0 | 0 | 0 | 0 | 0 | 33.199 | 0 |
| SPLEN2002053 | 0 | 0 | 0 | 0 | 0 | 63.499 | 0 |
| SPLEN2002133 | 0 | 0 | 0 | 0 | 0 | 72.687 | 0 |
| SPLEN2002147 | 0 | 0 | 0 | 0 | 0 | 100 | 0 |
| SPLEN2002166 | 0 | 0 | 0 | 0 | 0 | 100 | 0 |
| SPLEN2002175 | 0 | 0 | 0 | 0 | 0 | 100 | 0 |
| SPLEN2002223 | 0 | 0 | 0 | 0 | 0 | 60.831 | 0 |
| SPLEN2002272 | 0 | 0 | 0 | 0 | 0 | 32.545 | 0 |
| SPLEN2002314 | 0 | 0 | 0 | 0 | 0 | 100 | 0 |
| SPLEN2002335 | 0 | 0 | 0 | 0 | 0 | 3.523 | 0 |
| SPLEN2002343 | 0 | 0 | 0 | 0 | 0 | 100 | 0 |
| SPLEN2002354 | 0 | 0 | 0 | 0 | 0 | 100 | 0 |
| SPLEN2002385 | 0 | 0 | 0 | 0 | 0 | 100 | 0 |
| SPLEN2002419 | 0 | 0 | 0 | 0 | 0 | 100 | 0 |
| SPLEN2002451 | 0 | 0 | 0 | 0 | 0 | 67.599 | 0 |
| SPLEN2002462 | 0 | 0 | 0 | 0 | 0 | 100 | 0 |
| SPLEN2002463 | 0 | 0 | 0 | 0 | 0 | 13.174 | 0 |
| SPLEN2002467 | 0 | 0 | 0 | 0 | 0 | 5.667 | 0 |
| SPLEN2002477 | 0 | 0 | 0 | 0 | 5.734 | 45.229 | 0 |
| SPLEN2002493 | 0 | 0 | 0 | 0 | 0 | 3.011 | 0 |
| SPLEN2002662 | 0 | 0 | 0 | 0 | 0 | 100 | 0 |
| SPLEN2002695 | 0 | 0 | 0 | 0 | 0 | 100 | 0 |
| SPLEN2002707 | 0 | 0 | 0 | 0 | 0 | 100 | 0 |
| SPLEN2002744 | 0 | 0 | 0 | 0 | 0 | 3.880 | 0 |
| SPLEN2002917 | 0 | 0 | 0 | 0 | 0 | 100 | 0 |
| SPLEN2002931 | 0 | 0 | 0 | 0 | 0 | 7.094 | 0 |
| SPLEN2002957 | 0 | 0 | 0 | 0 | 0 | 100 | 0 |
| SPLEN2003160 | 0 | 0 | 0 | 0 | 0 | 100 | 0 |
| SPLEN2003204 | 0 | 0 | 0 | 0 | 0 | 100 | 0 |
| SPLEN2003219 | 0 | 0 | 0 | 0 | 0 | 100 | 0 |
| SPLEN2003297 | 0 | 0 | 0 | 0 | 0 | 46.191 | 0 |
| SPLEN2003396 | 0 | 0 | 0 | 0 | 0 | 32.545 | 0 |
| SPLEN2003678 | 0 | 0 | 0 | 0 | 0 | 61.070 | 0 |
| SPLEN2003878 | 0 | 0 | 0 | 0 | 0 | 100 | 0 |
| SPLEN2003918 | 0 | 0 | 0 | 0 | 0 | 100 | 0 |
| SPLEN2003924 | 0 | 0 | 0 | 0 | 0 | 100 | 0 |
| SPLEN2004060 | 0 | 0 | 0 | 0 | 0 | 100 | 0 |
| SPLEN2004078 | 0 | 0 | 0 | 0 | 0 | 25.996 | 0 |
| SPLEN2004124 | 0 | 0 | 0 | 0 | 0 | 100 | 0 |
| SPLEN2004148 | 0 | 0 | 0 | 0 | 0 | 100 | 0 |
| SPLEN2004181 | 0 | 0 | 0 | 0 | 0 | 13.695 | 0 |
| SPLEN2004220 | 0 | 0 | 0 | 0 | 0 | 10.007 | 0 |
| SPLEN2004343 | 0 | 0 | 0 | 0 | 0 | 31.298 | 0 |
| SPLEN2004346 | 0 | 0 | 0 | 0 | 0 | 100 | 0 |
| SPLEN2004368 | 0 | 0 | 0 | 0 | 0 | 100 | 0 |
| SPLEN2004555 | 0 | 0 | 8.297 | 0 | 0 | 3.703 | 0 |
| SPLEN2004611 | 0 | 0 | 0 | 0 | 0 | 67.599 | 0 |
| SPLEN2004662 | 0 | 0 | 0 | 0 | 0 | 65.533 | 0 |
| SPLEN2004773 | 0 | 0 | 0 | 0 | 0 | 3.714 | 0 |
| SPLEN2004844 | 0 | 0 | 0 | 0 | 0 | 19.862 | 0 |
| SPLEN2004880 | 95.022 | 0 | 0 | 0 | 0 | 1.025 | 2.004 |
| SPLEN2004984 | 0 | 0 | 0 | 0 | 0 | 13.708 | 0 |
| SPLEN2005009 | 0 | 0 | 0 | 0 | 0 | 14.955 | 0 |
| SPLEN2005227 | 0 | 0 | 0 | 0 | 0 | 100 | 0 |
| SPLEN2005272 | 0 | 0 | 0 | 0 | 0 | 100 | 0 |
| SPLEN2005342 | 0 | 0 | 0 | 0 | 0 | 9.281 | 0 |
| SPLEN2005416 | 0 | 0 | 0 | 0 | 0 | 60.831 | 0 |
| SPLEN2005429 | 0 | 0 | 0 | 0 | 0 | 6.373 | 0 |
| SPLEN2005450 | 0 | 0 | 0 | 0 | 0 | 100 | 0 |
| SPLEN2005474 | 0 | 0 | 0 | 0 | 0 | 100 | 0 |
| SPLEN2005560 | 0 | 0 | 0 | 0 | 0 | 1.831 | 0 |
| SPLEN2005727 | 0 | 0 | 0 | 0 | 0 | 100 | 0 |
| SPLEN2005783 | 0 | 0 | 0 | 0 | 0 | 11.340 | 0 |
| SPLEN2005790 | 0 | 0 | 0 | 0 | 0 | 67.599 | 0 |
| SPLEN2005806 | 0 | 0 | 0 | 0 | 0 | 2.128 | 0 |
| SPLEN2005818 | 0 | 0 | 0 | 0 | 0 | 38.781 | 0 |
| SPLEN2005838 | 0 | 0 | 0 | 0 | 0 | 63.033 | 0 |
| SPLEN2005869 | 0 | 0 | 0 | 0 | 0 | 11.172 | 0 |
| SPLEN2005927 | 0 | 0 | 33.569 | 0 | 15.196 | 7.491 | 0 |
| SPLEN2005939 | 0 | 0 | 0 | 0 | 0 | 100 | 0 |
| SPLEN2006122 | 0 | 0 | 0 | 0 | 0 | 2.930 | 0 |
| SPLEN2006133 | 0 | 0 | 0 | 6.514 | 6.695 | 6.601 | 0 |
| SPLEN2006143 | 0 | 0 | 0 | 0 | 0 | 3.892 | 0 |
| SPLEN2006232 | 0 | 0 | 0 | 0 | 0 | 14.970 | 0 |
| SPLEN2006268 | 0 | 0 | 0 | 0 | 0 | 100 | 0 |
| SPLEN2006283 | 0 | 0 | 0 | 0 | 0 | 100 | 0 |
| SPLEN2006305 | 0 | 0 | 0 | 4.337 | 4.458 | 8.791 | 0 |
| SPLEN2006325 | 0 | 0 | 0 | 0 | 0 | 14.493 | 0 |
| SPLEN2006374 | 0 | 0 | 0 | 0 | 0 | 2.644 | 0 |
| SPLEN2006389 | 0 | 0 | 0 | 0 | 0 | 100 | 0 |
| SPLEN2006425 | 0 | 0 | 0 | 0 | 0 | 100 | 0 |
| SPLEN2006671 | 0 | 0 | 0 | 0 | 0 | 32.498 | 0 |
| SPLEN2006701 | 0 | 0 | 0 | 0 | 0 | 100 | 0 |
| SPLEN2006785 | 0 | 0 | 0 | 0 | 0 | 100 | 0 |
| SPLEN2006875 | 0 | 0 | 0 | 0 | 0 | 100 | 0 |
| SPLEN2007182 | 0 | 0 | 0 | 0 | 0 | 22.235 | 0 |
| SPLEN2007276 | 0 | 0 | 0 | 0 | 0 | 4.614 | 0 |
| SPLEN2007314 | 0 | 0 | 0 | 0 | 0 | 100 | 0 |
| SPLEN2007326 | 0 | 0 | 0 | 0 | 0 | 67.599 | 0 |
| SPLEN2007350 | 0 | 0 | 0 | 0 | 0 | 100 | 0 |
| SPLEN2007388 | 0 | 0 | 0 | 0 | 0 | 46.191 | 0 |
| SPLEN2007498 | 0 | 0 | 0 | 0 | 0 | 4.553 | 0 |
| SPLEN2007619 | 0 | 0 | 0 | 0 | 0 | 100 | 0 |
| SPLEN2007653 | 0 | 0 | 0 | 0 | 0 | 100 | 0 |
| SPLEN2007689 | 0 | 0 | 0 | 0 | 0 | 100 | 0 |
| SPLEN2007695 | 0 | 0 | 0 | 0 | 0 | 100 | 0 |
| SPLEN2007739 | 0 | 0 | 0 | 0 | 0 | 10.563 | 0 |
| SPLEN2007750 | 0 | 0 | 0 | 0 | 0 | 16.833 | 0 |
| SPLEN2007794 | 0 | 0 | 0 | 0 | 0 | 17.241 | 0 |
| SPLEN2007879 | 0 | 0 | 0 | 0 | 50.875 | 25.081 | 0 |
| SPLEN2007926 | 0 | 0 | 0 | 0 | 0 | 100 | 0 |
| SPLEN2007951 | 0 | 0 | 0 | 0 | 0 | 41.102 | 0 |
| SPLEN2008007 | 0 | 0 | 0 | 0 | 0 | 100 | 0 |
| SPLEN2008135 | 0 | 0 | 0 | 0 | 0 | 100 | 0 |
| SPLEN2008164 | 0 | 0 | 0 | 0 | 0 | 100 | 0 |
| SPLEN2008317 | 0 | 0 | 0 | 0 | 0 | 100 | 0 |
| SPLEN2008460 | 0 | 0 | 0 | 0 | 0 | 4.558 | 0 |
| SPLEN2008496 | 0 | 0 | 0 | 0 | 0 | 13.469 | 0 |
| SPLEN2008591 | 0 | 0 | 0 | 0 | 0 | 72.687 | 0 |
| SPLEN2008786 | 0 | 0 | 0 | 0 | 0 | 100 | 0 |
| SPLEN2009081 | 0 | 0 | 0 | 0 | 0 | 11.259 | 0 |
| SPLEN2009088 | 0 | 0 | 0 | 0 | 0 | 100 | 0 |
| SPLEN2009163 | 0 | 0 | 0 | 0 | 0 | 31.971 | 0 |
| SPLEN2009315 | 0 | 0 | 0 | 0 | 0 | 59.326 | 0 |
| SPLEN2009340 | 0 | 0 | 0 | 0 | 0 | 100 | 0 |
| SPLEN2009512 | 0 | 0 | 0 | 0 | 0 | 28.811 | 0 |
| SPLEN2009513 | 0 | 0 | 0 | 0 | 0 | 10.785 | 42.191 |
| SPLEN2009541 | 0 | 0 | 0 | 0 | 0 | 100 | 0 |
| SPLEN2009548 | 0 | 0 | 0 | 0 | 0 | 100 | 0 |
| SPLEN2009555 | 0 | 0 | 0 | 0 | 0 | 14.776 | 0 |
| SPLEN2009581 | 0 | 0 | 0 | 0 | 0 | 100 | 0 |
| SPLEN2009733 | 0 | 0 | 0 | 0 | 0 | 100 | 0 |
| SPLEN2009803 | 0 | 0 | 0 | 0 | 0 | 100 | 0 |
| SPLEN2009884 | 0 | 0 | 0 | 0 | 0 | 100 | 0 |
| SPLEN2009918 | 0 | 0 | 0 | 0 | 0 | 100 | 0 |
| SPLEN2009970 | 0 | 0 | 0 | 0 | 0 | 8.872 | 0 |
| SPLEN2010004 | 0 | 0 | 0 | 0 | 0 | 100 | 0 |
| SPLEN2010015 | 0 | 0 | 0 | 0 | 0 | 27.044 | 0 |
| SPLEN2010112 | 0 | 0 | 0 | 0 | 0 | 100 | 0 |
| SPLEN2010119 | 0 | 0 | 0 | 0 | 0 | 100 | 0 |
| SPLEN2010146 | 0 | 0 | 0 | 0 | 0 | 100 | 0 |
| SPLEN2010187 | 0 | 0 | 0 | 0 | 0 | 100 | 0 |
| SPLEN2010195 | 0 | 0 | 0 | 0 | 0 | 2.025 | 0 |
| SPLEN2010350 | 0 | 0 | 0 | 0 | 0 | 100 | 0 |
| SPLEN2010395 | 0 | 0 | 0 | 0 | 0 | 100 | 0 |
| SPLEN2010415 | 0 | 0 | 0 | 0 | 0 | 100 | 0 |
| SPLEN2010447 | 0 | 0 | 0 | 0 | 0 | 100 | 0 |
| SPLEN2010469 | 0 | 0 | 0 | 0 | 0 | 43.126 | 0 |
| SPLEN2010510 | 0 | 0 | 0 | 0 | 0 | 100 | 0 |
| SPLEN2010530 | 0 | 0 | 0 | 0 | 0 | 100 | 0 |
| SPLEN2010534 | 0 | 0 | 0 | 0 | 0 | 100 | 0 |
| SPLEN2010588 | 0 | 0 | 0 | 0 | 0 | 100 | 0 |
| SPLEN2010625 | 0 | 0 | 0 | 0 | 0 | 67.599 | 0 |
| SPLEN2010800 | 0 | 0 | 0 | 0 | 0 | 100 | 0 |
| SPLEN2010846 | 0 | 0 | 0 | 0 | 0 | 39.963 | 0 |
| SPLEN2010847 | 0 | 0 | 0 | 0 | 0 | 28.694 | 0 |
| SPLEN2010862 | 0 | 0 | 0 | 0 | 0 | 100 | 0 |
| SPLEN2010912 | 0 | 0 | 0 | 0 | 0 | 21.546 | 0 |
| SPLEN2010978 | 0 | 0 | 0 | 0 | 0 | 100 | 0 |
| SPLEN2010986 | 0 | 0 | 0 | 0 | 0 | 35.034 | 0 |
| SPLEN2011018 | 0 | 0 | 0 | 0 | 0 | 100 | 0 |
| SPLEN2011021 | 0 | 0 | 0 | 0 | 57.827 | 28.508 | 0 |
| SPLEN2011025 | 0 | 0 | 0 | 0 | 66.980 | 33.020 | 0 |
| SPLEN2011086 | 0 | 0 | 0 | 0 | 0 | 100 | 0 |
| SPLEN2011139 | 0 | 0 | 0 | 0 | 0 | 100 | 0 |
| SPLEN2011145 | 0 | 0 | 0 | 0 | 0 | 100 | 0 |
| SPLEN2011248 | 0 | 0 | 0 | 0 | 0 | 100 | 0 |
| SPLEN2011252 | 0 | 0 | 0 | 0 | 0 | 100 | 0 |
| SPLEN2011419 | 0 | 0 | 0 | 0 | 0 | 4.157 | 0 |
| SPLEN2011422 | 0 | 0 | 0 | 0 | 0 | 100 | 0 |
| SPLEN2011672 | 0 | 0 | 0 | 0 | 0 | 100 | 0 |
| SPLEN2011678 | 0 | 0 | 0 | 0 | 0 | 1.983 | 0 |
| SPLEN2011737 | 0 | 0 | 0 | 0 | 0 | 9.065 | 0 |
| SPLEN2011758 | 0 | 0 | 0 | 0 | 0 | 18.273 | 0 |
| SPLEN2011766 | 0 | 0 | 0 | 0 | 0 | 28.426 | 0 |
| SPLEN2011805 | 0 | 0 | 0 | 0 | 0 | 100 | 0 |
| SPLEN2011820 | 0 | 0 | 0 | 0 | 0 | 100 | 0 |
| SPLEN2011830 | 0 | 0 | 0 | 0 | 0 | 100 | 0 |
| SPLEN2011931 | 0 | 0 | 0 | 0 | 0 | 100 | 0 |
| SPLEN2011981 | 0 | 0 | 0 | 0 | 0 | 100 | 0 |
| SPLEN2012115 | 0 | 0 | 0 | 0 | 0 | 7.120 | 0 |
| SPLEN2012175 | 0 | 0 | 0 | 0 | 0 | 100 | 0 |
| SPLEN2012179 | 0 | 0 | 0 | 0 | 0 | 100 | 0 |
| SPLEN2012185 | 0 | 0 | 0 | 0 | 0 | 100 | 0 |
| SPLEN2012234 | 0 | 0 | 0 | 0 | 0 | 100 | 0 |
| SPLEN2012453 | 0 | 0 | 0 | 0 | 0 | 71.023 | 0 |
| SPLEN2012523 | 0 | 0 | 0 | 0 | 0 | 1.857 | 7.266 |
| SPLEN2012524 | 0 | 0 | 0 | 0 | 0 | 100 | 0 |
| SPLEN2012525 | 0 | 0 | 0 | 0 | 0 | 100 | 0 |
| SPLEN2012571 | 0 | 0 | 0 | 0 | 11.218 | 5.530 | 0 |
| SPLEN2012606 | 0 | 0 | 0 | 0 | 0 | 100 | 0 |
| SPLEN2012611 | 0 | 0 | 0 | 0 | 0 | 49.950 | 0 |
| SPLEN2012619 | 0 | 0 | 0 | 0 | 0 | 63.499 | 0 |
| SPLEN2012624 | 0 | 0 | 0 | 0 | 0 | 17.593 | 0 |
| SPLEN2012725 | 0 | 0 | 0 | 0 | 0 | 100 | 0 |
| SPLEN2012743 | 0 | 0 | 0 | 0 | 0 | 100 | 0 |
| SPLEN2012756 | 0 | 0 | 0 | 0 | 0 | 100 | 0 |
| SPLEN2012800 | 0 | 0 | 0 | 0 | 0 | 0.594 | 0 |
| SPLEN2012846 | 0 | 0 | 0 | 0 | 0 | 47.098 | 0 |
| SPLEN2012889 | 0 | 0 | 0 | 0 | 0 | 67.599 | 0 |
| SPLEN2012961 | 0 | 0 | 0 | 0 | 0 | 100 | 0 |
| SPLEN2013108 | 0 | 0 | 0 | 0 | 0 | 60.831 | 0 |
| SPLEN2013195 | 0 | 0 | 0 | 0 | 0 | 71.023 | 0 |
| SPLEN2013503 | 0 | 0 | 0 | 0 | 0 | 13.099 | 0 |
| SPLEN2013644 | 0 | 0 | 0 | 0 | 0 | 100 | 0 |
| SPLEN2013648 | 0 | 0 | 0 | 0 | 0 | 100 | 0 |
| SPLEN2013670 | 0 | 0 | 0 | 0 | 0 | 20.398 | 0 |
| SPLEN2013673 | 0 | 0 | 0 | 0 | 0 | 100 | 0 |
| SPLEN2013690 | 0 | 0 | 0 | 0 | 0 | 18.787 | 0 |
| SPLEN2013714 | 0 | 0 | 0 | 0 | 0 | 100 | 0 |
| SPLEN2013753 | 0 | 0 | 0 | 0 | 0 | 67.599 | 0 |
| SPLEN2013821 | 0 | 0 | 0 | 0 | 0 | 12.968 | 0 |
| SPLEN2013857 | 0 | 0 | 0 | 0 | 0 | 63.499 | 0 |
| SPLEN2013860 | 0 | 0 | 0 | 0 | 0 | 100 | 0 |
| SPLEN2013910 | 0 | 0 | 0 | 0 | 0 | 100 | 0 |
| SPLEN2013923 | 0 | 0 | 0 | 0 | 0 | 47.098 | 0 |
| SPLEN2013936 | 0 | 0 | 0 | 0 | 0 | 11.607 | 45.406 |
| SPLEN2014041 | 0 | 0 | 0 | 0 | 0 | 67.599 | 0 |
| SPLEN2014063 | 0 | 0 | 0 | 0 | 0 | 100 | 0 |
| SPLEN2014073 | 0 | 0 | 0 | 0 | 0 | 20.525 | 0 |
| SPLEN2014080 | 0 | 0 | 0 | 0 | 0 | 8.242 | 0 |
| SPLEN2014119 | 0 | 0 | 0 | 0 | 0 | 29.033 | 0 |
| SPLEN2014136 | 0 | 0 | 0 | 0 | 0 | 100 | 0 |
| SPLEN2014199 | 0 | 0 | 0 | 0 | 0 | 100 | 0 |
| SPLEN2014210 | 0 | 0 | 0 | 0 | 0 | 100 | 0 |
| SPLEN2014293 | 0 | 0 | 0 | 0 | 0 | 100 | 0 |
| SPLEN2014318 | 0 | 0 | 0 | 0 | 0 | 100 | 0 |
| SPLEN2014381 | 0 | 0 | 0 | 0 | 0 | 25.867 | 0 |
| SPLEN2014452 | 0 | 0 | 0 | 0 | 0 | 31.971 | 0 |
| SPLEN2014454 | 0 | 0 | 0 | 0 | 0 | 100 | 0 |
| SPLEN2014486 | 0 | 0 | 0 | 0 | 0 | 100 | 0 |
| SPLEN2014543 | 0 | 0 | 0 | 0 | 0 | 100 | 0 |
| SPLEN2014572 | 0 | 0 | 0 | 0 | 0 | 10.827 | 0 |
| SPLEN2014645 | 0 | 0 | 0 | 0 | 0 | 31.852 | 0 |
| SPLEN2014669 | 0 | 0 | 0 | 0 | 0 | 100 | 0 |
| SPLEN2014711 | 0 | 0 | 0 | 0 | 0 | 4.614 | 0 |
| SPLEN2014739 | 0 | 0 | 0 | 0 | 0 | 75.784 | 0 |
| SPLEN2014860 | 0 | 0 | 0 | 0 | 0 | 100 | 0 |
| SPLEN2014865 | 0 | 0 | 0 | 0 | 0 | 100 | 0 |
| SPLEN2014911 | 0 | 0 | 0 | 0 | 3.723 | 9.176 | 7.179 |
| SPLEN2014919 | 0 | 0 | 0 | 0 | 0 | 100 | 0 |
| SPLEN2014920 | 0 | 0 | 0 | 0 | 0 | 100 | 0 |
| SPLEN2014924 | 0 | 0 | 0 | 0 | 0 | 100 | 0 |
| SPLEN2014937 | 0 | 0 | 0 | 0 | 0 | 100 | 0 |
| SPLEN2014946 | 0 | 0 | 0 | 0 | 0 | 100 | 0 |
| SPLEN2015031 | 0 | 0 | 0 | 0 | 0 | 100 | 0 |
| SPLEN2015094 | 0 | 0 | 0 | 0 | 0 | 100 | 0 |
| SPLEN2015121 | 0 | 0 | 0 | 0 | 0 | 67.599 | 0 |
| SPLEN2015129 | 0 | 0 | 0 | 0 | 0 | 63.499 | 0 |
| SPLEN2015158 | 0 | 0 | 0 | 0 | 0 | 100 | 0 |
| SPLEN2015166 | 0 | 0 | 0 | 0 | 66.980 | 33.020 | 0 |
| SPLEN2015216 | 0 | 0 | 0 | 0 | 0 | 100 | 0 |
| SPLEN2015258 | 0 | 0 | 0 | 0 | 0 | 100 | 0 |
| SPLEN2015261 | 0 | 0 | 0 | 0 | 0 | 100 | 0 |
| SPLEN2015267 | 0 | 0 | 0 | 0 | 0 | 100 | 0 |
| SPLEN2015276 | 0 | 0 | 0 | 0 | 0 | 100 | 0 |
| SPLEN2015310 | 0 | 0 | 0 | 3.936 | 6.068 | 0.997 | 0 |
| SPLEN2015415 | 0 | 0 | 0 | 0 | 0 | 100 | 0 |
| SPLEN2015433 | 0 | 0 | 0 | 0 | 0 | 100 | 0 |
| SPLEN2015593 | 0 | 0 | 0 | 0 | 0 | 100 | 0 |
| SPLEN2015674 | 0 | 0 | 0 | 0 | 0 | 100 | 0 |
| SPLEN2015679 | 0 | 0 | 0 | 0 | 0 | 100 | 0 |
| SPLEN2015707 | 0 | 0 | 0 | 0 | 0 | 100 | 0 |
| SPLEN2015730 | 0 | 0 | 0 | 0 | 0 | 48.681 | 0 |
| SPLEN2015746 | 0 | 0 | 0 | 0 | 0 | 100 | 0 |
| SPLEN2015788 | 0 | 0 | 0 | 0 | 0 | 25.263 | 32.942 |
| SPLEN2015815 | 0 | 0 | 0 | 0 | 0 | 100 | 0 |
| SPLEN2015890 | 0 | 0 | 0 | 0 | 0 | 71.023 | 0 |
| SPLEN2015891 | 0 | 0 | 0 | 0 | 0 | 100 | 0 |
| SPLEN2015899 | 0 | 0 | 0 | 0 | 0 | 100 | 0 |
| SPLEN2016045 | 0 | 0 | 0 | 0 | 0 | 100 | 0 |
| SPLEN2016053 | 0 | 0 | 0 | 0 | 0 | 100 | 0 |
| SPLEN2016069 | 0 | 0 | 0 | 0 | 0 | 100 | 0 |
| SPLEN2016098 | 0 | 0 | 0 | 0 | 0 | 31.971 | 0 |
| SPLEN2016135 | 0 | 0 | 0 | 0 | 0 | 100 | 0 |
| SPLEN2016139 | 0 | 0 | 0 | 0 | 0 | 2.229 | 0 |
| SPLEN2016241 | 0 | 0 | 0 | 0 | 0 | 100 | 0 |
| SPLEN2016250 | 0 | 0 | 0 | 0 | 0 | 100 | 0 |
| SPLEN2016268 | 0 | 0 | 0 | 2.966 | 1.525 | 1.503 | 0 |
| SPLEN2016304 | 0 | 0 | 0 | 0 | 0 | 100 | 0 |
| SPLEN2016356 | 0 | 0 | 0 | 0 | 0 | 100 | 0 |
| SPLEN2016421 | 0 | 0 | 0 | 0 | 7.310 | 7.208 | 0 |
| SPLEN2016480 | 0 | 0 | 0 | 0 | 0 | 100 | 0 |
| SPLEN2016531 | 0 | 0 | 0 | 0 | 0 | 100 | 0 |
| SPLEN2016533 | 0 | 0 | 0 | 0 | 0 | 61.070 | 0 |
| SPLEN2016541 | 0 | 0 | 0 | 0 | 0 | 100 | 0 |
| SPLEN2016552 | 0 | 0 | 0 | 0 | 0 | 100 | 0 |
| SPLEN2016554 | 0 | 0 | 0 | 0 | 0 | 32.422 | 0 |
| SPLEN2016602 | 0 | 0 | 0 | 0 | 0 | 100 | 0 |
| SPLEN2016627 | 0 | 0 | 0 | 0 | 0 | 100 | 0 |
| SPLEN2016678 | 0 | 0 | 0 | 0 | 0 | 34.278 | 0 |
| SPLEN2016743 | 0 | 0 | 0 | 0 | 20.232 | 9.974 | 0 |
| SPLEN2016773 | 0 | 0 | 0 | 0 | 0 | 71.023 | 0 |
| SPLEN2016781 | 0 | 0 | 0 | 0 | 0 | 67.599 | 0 |
| SPLEN2016840 | 0 | 0 | 0 | 0 | 0 | 100 | 0 |
| SPLEN2016863 | 0 | 0 | 0 | 0 | 0 | 100 | 0 |
| SPLEN2016922 | 0 | 0 | 0 | 0 | 0 | 100 | 0 |
| SPLEN2016932 | 0 | 0 | 0 | 0 | 0 | 61.070 | 0 |
| SPLEN2016972 | 0 | 0 | 0 | 13.923 | 0 | 3.528 | 0 |
| SPLEN2016973 | 0 | 0 | 0 | 0 | 0 | 100 | 0 |
| SPLEN2017043 | 0 | 0 | 0 | 0 | 0 | 18.106 | 0 |
| SPLEN2017104 | 0 | 0 | 0 | 0 | 0 | 100 | 0 |
| SPLEN2017121 | 0 | 0 | 0 | 0 | 0 | 13.487 | 0 |
| SPLEN2017147 | 0 | 0 | 0 | 0 | 0 | 100 | 0 |
| SPLEN2017187 | 0 | 0 | 0 | 0 | 0 | 100 | 0 |
| SPLEN2017189 | 0 | 0 | 0 | 0 | 0 | 100 | 0 |
| SPLEN2017212 | 0 | 0 | 0 | 0 | 0 | 100 | 0 |
| SPLEN2017318 | 0 | 0 | 0 | 0 | 17.905 | 8.827 | 0 |
| SPLEN2017351 | 0 | 0 | 0 | 0 | 0 | 5.233 | 0 |
| SPLEN2017426 | 0 | 0 | 0 | 0 | 0 | 100 | 0 |
| SPLEN2017452 | 0 | 0 | 0 | 0 | 0 | 100 | 0 |
| SPLEN2017538 | 0 | 0 | 0 | 0 | 0 | 100 | 0 |
| SPLEN2017592 | 0 | 0 | 0 | 0 | 0 | 71.023 | 0 |
| SPLEN2017613 | 0 | 0 | 0 | 0 | 0 | 100 | 0 |
| SPLEN2017620 | 0 | 0 | 0 | 0 | 0 | 8.917 | 0 |
| SPLEN2017643 | 0 | 0 | 0 | 0 | 0 | 100 | 0 |
| SPLEN2017687 | 0 | 0 | 0 | 0 | 0 | 100 | 0 |
| SPLEN2017740 | 0 | 0 | 0 | 0 | 0 | 100 | 0 |
| SPLEN2017781 | 0 | 0 | 0 | 0 | 0 | 71.023 | 0 |
| SPLEN2017918 | 0 | 0 | 0 | 0 | 3.217 | 4.758 | 0 |
| SPLEN2017999 | 0 | 0 | 0 | 0 | 0 | 100 | 0 |
| SPLEN2018098 | 0 | 0 | 0 | 0 | 0 | 59.326 | 0 |
| SPLEN2018112 | 0 | 0 | 0 | 0 | 0 | 100 | 0 |
| SPLEN2018157 | 0 | 0 | 0 | 0 | 0 | 12.588 | 16.414 |
| SPLEN2018159 | 0 | 0 | 0 | 0 | 0 | 16.475 | 0 |
| SPLEN2018180 | 0 | 0 | 0 | 0 | 0 | 100 | 0 |
| SPLEN2018181 | 0 | 0 | 0 | 0 | 0 | 100 | 0 |
| SPLEN2018280 | 0 | 0 | 0 | 0 | 0 | 100 | 0 |
| SPLEN2018285 | 0 | 0 | 0 | 0 | 0 | 100 | 0 |
| SPLEN2018299 | 0 | 0 | 0 | 0 | 0 | 35.797 | 0 |
| SPLEN2018302 | 0 | 42.846 | 0 | 0 | 0 | 10.578 | 0 |
| SPLEN2018364 | 0 | 0 | 0 | 0 | 0 | 100 | 0 |
| SPLEN2018395 | 0 | 0 | 0 | 0 | 0 | 100 | 0 |
| SPLEN2018643 | 0 | 0 | 0 | 0 | 0 | 63.499 | 0 |
| SPLEN2018683 | 0 | 0 | 0 | 0 | 0 | 67.599 | 0 |
| SPLEN2018732 | 0 | 0 | 0 | 0 | 0 | 100 | 0 |
| SPLEN2018749 | 0 | 0 | 0 | 0 | 0 | 100 | 0 |
| SPLEN2018933 | 0 | 0 | 0 | 0 | 0 | 100 | 0 |
| SPLEN2019008 | 0 | 0 | 0 | 0 | 0 | 100 | 0 |
| SPLEN2019043 | 0 | 0 | 0 | 0 | 0 | 100 | 0 |
| SPLEN2019077 | 0 | 0 | 0 | 0 | 0 | 18.457 | 0 |
| SPLEN2019092 | 0 | 0 | 0 | 0 | 0 | 48.294 | 0 |
| SPLEN2019102 | 0 | 0 | 0 | 0 | 0 | 100 | 0 |
| SPLEN2019131 | 0 | 0 | 0 | 0 | 0 | 100 | 0 |
| SPLEN2019169 | 0 | 0 | 0 | 0 | 0 | 9.849 | 0 |
| SPLEN2019257 | 0 | 0 | 0 | 0 | 0 | 71.023 | 0 |
| SPLEN2019311 | 0 | 0 | 0 | 0 | 0 | 42.173 | 0 |
| SPLEN2019323 | 0 | 0 | 0 | 0 | 0 | 100 | 0 |
| SPLEN2019349 | 0 | 0 | 0 | 0 | 0 | 63.033 | 0 |
| SPLEN2019375 | 0 | 0 | 0 | 0 | 0 | 100 | 0 |
| SPLEN2019379 | 0 | 0 | 0 | 0 | 0 | 19.862 | 0 |
| SPLEN2019405 | 0 | 0 | 0 | 0 | 0 | 7.474 | 0 |
| SPLEN2019446 | 0 | 0 | 0 | 0 | 0 | 18.102 | 0 |
| SPLEN2019471 | 0 | 0 | 0 | 0 | 0 | 100 | 0 |
| SPLEN2019480 | 0 | 0 | 0 | 0 | 0 | 100 | 0 |
| SPLEN2019505 | 0 | 0 | 0 | 0 | 0 | 8.528 | 0 |
| SPLEN2019571 | 0 | 0 | 0 | 0 | 0 | 100 | 0 |
| SPLEN2019575 | 0 | 0 | 0 | 0 | 0 | 100 | 0 |
| SPLEN2019709 | 0 | 0 | 0 | 0 | 0 | 100 | 0 |
| SPLEN2019774 | 0 | 0 | 0 | 0 | 0 | 15.455 | 0 |
| SPLEN2019793 | 0 | 0 | 0 | 0 | 0 | 100 | 0 |
| SPLEN2019811 | 0 | 0 | 0 | 0 | 0 | 100 | 0 |
| SPLEN2019839 | 0 | 0 | 0 | 0 | 0 | 100 | 0 |
| SPLEN2019956 | 0 | 0 | 0 | 0 | 0 | 52.985 | 0 |
| SPLEN2019985 | 0 | 0 | 0 | 0 | 0 | 100 | 0 |
| SPLEN2020007 | 0 | 0 | 0 | 0 | 0 | 32.498 | 0 |
| SPLEN2020034 | 0 | 0 | 0 | 0 | 0 | 43.126 | 0 |
| SPLEN2020153 | 0 | 0 | 0 | 0 | 0 | 100 | 0 |
| SPLEN2020154 | 0 | 0 | 0 | 0 | 0 | 100 | 0 |
| SPLEN2020166 | 0 | 0 | 0 | 0 | 0 | 100 | 0 |
| SPLEN2020183 | 0 | 0 | 0 | 0 | 0 | 100 | 0 |
| SPLEN2020223 | 0 | 0 | 0 | 0 | 0 | 100 | 0 |
| SPLEN2020359 | 0 | 0 | 0 | 0 | 0 | 100 | 0 |
| SPLEN2020417 | 0 | 0 | 0 | 0 | 0 | 24.283 | 0 |
| SPLEN2020427 | 0 | 0 | 0 | 0 | 0 | 100 | 0 |
| SPLEN2020467 | 0 | 0 | 0 | 0 | 0 | 100 | 0 |
| SPLEN2020512 | 0 | 0 | 0 | 0 | 0 | 100 | 0 |
| SPLEN2020608 | 0 | 0 | 0 | 0 | 0 | 100 | 0 |
| SPLEN2020777 | 0 | 0 | 0 | 0 | 0 | 100 | 0 |
| SPLEN2020805 | 0 | 0 | 0 | 0 | 0 | 100 | 0 |
| SPLEN2021057 | 0 | 0 | 0 | 0 | 0 | 100 | 0 |
| SPLEN2021122 | 0 | 0 | 0 | 0 | 0 | 28.373 | 0 |
| SPLEN2021157 | 0 | 0 | 0 | 0 | 0 | 100 | 0 |
| SPLEN2021194 | 0 | 0 | 0 | 0 | 0 | 5.868 | 0 |
| SPLEN2021231 | 0 | 0 | 0 | 0 | 0 | 100 | 0 |
| SPLEN2021273 | 0 | 0 | 0 | 0 | 0 | 51.310 | 0 |
| SPLEN2021295 | 0 | 0 | 0 | 0 | 0 | 100 | 0 |
| SPLEN2021298 | 0 | 0 | 0 | 0 | 0 | 100 | 0 |
| SPLEN2021383 | 0 | 0 | 0 | 0 | 0 | 100 | 0 |
| SPLEN2021417 | 0 | 0 | 0 | 0 | 0 | 100 | 0 |
| SPLEN2021440 | 0 | 0 | 0 | 0 | 0 | 100 | 0 |
| SPLEN2021458 | 0 | 0 | 0 | 0 | 0 | 61.070 | 0 |
| SPLEN2021463 | 0 | 0 | 0 | 0 | 0 | 100 | 0 |
| SPLEN2021560 | 0 | 0 | 0 | 0 | 0 | 100 | 0 |
| SPLEN2021701 | 0 | 0 | 0 | 0 | 0 | 100 | 0 |
| SPLEN2021795 | 0 | 0 | 0 | 0 | 0 | 100 | 0 |
| SPLEN2021983 | 0 | 0 | 0 | 0 | 0 | 100 | 0 |
| SPLEN2021991 | 0 | 0 | 0 | 0 | 0 | 100 | 0 |
| SPLEN2022040 | 0 | 0 | 0 | 0 | 0 | 100 | 0 |
| SPLEN2022162 | 0 | 0 | 0 | 0 | 0 | 100 | 0 |
| SPLEN2022227 | 0 | 0 | 0 | 0 | 0 | 100 | 0 |
| SPLEN2022522 | 0 | 0 | 0 | 0 | 0 | 4.932 | 0 |
| SPLEN2022591 | 0 | 0 | 0 | 0 | 0 | 100 | 0 |
| SPLEN2022785 | 0 | 0 | 0 | 0 | 0 | 100 | 0 |
| SPLEN2022920 | 0 | 0 | 0 | 0 | 0 | 100 | 0 |
| SPLEN2023024 | 0 | 0 | 0 | 0 | 0 | 100 | 0 |
| SPLEN2023118 | 0 | 0 | 0 | 0 | 0 | 19.364 | 0 |
| SPLEN2023160 | 0 | 0 | 0 | 0 | 0 | 34.195 | 0 |
| SPLEN2023423 | 0 | 0 | 0 | 0 | 0 | 100 | 0 |
| SPLEN2023710 | 0 | 0 | 0 | 0 | 0 | 100 | 0 |
| SPLEN2023733 | 0 | 0 | 0 | 0 | 0 | 70.051 | 0 |
| SPLEN2023791 | 0 | 0 | 0 | 0 | 0 | 49.431 | 0 |
| SPLEN2023977 | 0 | 0 | 0 | 0 | 0 | 100 | 0 |
| SPLEN2024070 | 0 | 0 | 0 | 0 | 0 | 100 | 0 |
| SPLEN2024127 | 0 | 0 | 0 | 0 | 0 | 100 | 0 |
| SPLEN2024232 | 0 | 0 | 0 | 0 | 0 | 11.539 | 0 |
| SPLEN2024273 | 0 | 0 | 0 | 0 | 0 | 100 | 0 |
| SPLEN2024383 | 0 | 0 | 0 | 0 | 0 | 100 | 0 |
| SPLEN2024489 | 0 | 0 | 0 | 0 | 0 | 19.007 | 0 |
| SPLEN2024530 | 0 | 0 | 0 | 0 | 0 | 100 | 0 |
| SPLEN2024571 | 0 | 0 | 0 | 0 | 0 | 100 | 0 |
| SPLEN2024577 | 0 | 0 | 0 | 0 | 0 | 100 | 0 |
| SPLEN2024617 | 0 | 0 | 0 | 0 | 0 | 100 | 0 |
| SPLEN2024737 | 0 | 0 | 0 | 0 | 0 | 100 | 0 |
| SPLEN2024922 | 0 | 0 | 0 | 0 | 0 | 44.836 | 0 |
| SPLEN2024956 | 0 | 0 | 0 | 0 | 0 | 100 | 0 |
| SPLEN2024990 | 0 | 0 | 0 | 0 | 0 | 80.667 | 0 |
| SPLEN2025012 | 0 | 0 | 0 | 0 | 0 | 100 | 0 |
| SPLEN2025017 | 0 | 0 | 0 | 0 | 0 | 58.340 | 0 |
| SPLEN2025039 | 0 | 0 | 0 | 0 | 0 | 71.023 | 0 |
| SPLEN2025219 | 0 | 0 | 0 | 0 | 0 | 100 | 0 |
| SPLEN2025491 | 0 | 0 | 0 | 0 | 0 | 61.070 | 0 |
| SPLEN2025626 | 0 | 0 | 0 | 0 | 0 | 100 | 0 |
| SPLEN2025820 | 0 | 0 | 0 | 0 | 0 | 100 | 0 |
| SPLEN2026144 | 0 | 0 | 0 | 0 | 0 | 100 | 0 |
| SPLEN2026411 | 0 | 0 | 0 | 0 | 0 | 19.827 | 0 |
| SPLEN2026765 | 0 | 0 | 0 | 0 | 0 | 100 | 0 |
| SPLEN2027113 | 0 | 0 | 0 | 0 | 0 | 2.847 | 0 |
| SPLEN2027157 | 0 | 0 | 0 | 0 | 0 | 100 | 0 |
| SPLEN2027395 | 0 | 0 | 0 | 0 | 0 | 100 | 0 |
| SPLEN2027488 | 0 | 0 | 0 | 0 | 0 | 100 | 0 |
| SPLEN2027780 | 0 | 0 | 0 | 0 | 0 | 48.294 | 0 |
| SPLEN2027852 | 0 | 0 | 0 | 0 | 0 | 100 | 0 |
| SPLEN2027868 | 0 | 0 | 0 | 0 | 0 | 100 | 0 |
| SPLEN2027995 | 0 | 0 | 0 | 0 | 0 | 100 | 0 |
| SPLEN2028046 | 0 | 0 | 0 | 0 | 0 | 100 | 0 |
| SPLEN2028066 | 0 | 0 | 0 | 0 | 0 | 100 | 0 |
| SPLEN2028138 | 0 | 0 | 0 | 0 | 0 | 100 | 0 |
| SPLEN2028365 | 0 | 0 | 0 | 0 | 0 | 5.970 | 0 |
| SPLEN2028417 | 0 | 0 | 0 | 0 | 0 | 100 | 0 |
| SPLEN2028424 | 0 | 0 | 0 | 0 | 0 | 48.497 | 0 |
| SPLEN2028466 | 0 | 0 | 0 | 0 | 2.919 | 1.439 | 0 |
| SPLEN2028593 | 0 | 0 | 0 | 0 | 0 | 100 | 0 |
| SPLEN2028844 | 0 | 0 | 0 | 0 | 0 | 100 | 0 |
| SPLEN2028914 | 0 | 0 | 0 | 0 | 0 | 4.858 | 0 |
| SPLEN2028994 | 0 | 0 | 0 | 0 | 0 | 100 | 0 |
| SPLEN2029051 | 0 | 0 | 0 | 0 | 0 | 100 | 0 |
| SPLEN2029176 | 0 | 0 | 0 | 0 | 0 | 100 | 0 |
| SPLEN2029295 | 0 | 0 | 0 | 0 | 0 | 60.831 | 0 |
| SPLEN2029380 | 0 | 0 | 0 | 0 | 0 | 100 | 0 |
| SPLEN2029522 | 0 | 0 | 0 | 0 | 0 | 100 | 0 |
| SPLEN2029683 | 0 | 0 | 0 | 0 | 0 | 60.831 | 0 |
| SPLEN2029727 | 0 | 0 | 0 | 0 | 0 | 100 | 0 |
| SPLEN2029904 | 0 | 0 | 0 | 0 | 0 | 100 | 0 |
| SPLEN2029912 | 0 | 11.510 | 0 | 0 | 0 | 2.842 | 0 |
| SPLEN2030250 | 0 | 0 | 0 | 0 | 0 | 100 | 0 |
| SPLEN2030335 | 0 | 0 | 0 | 0 | 0 | 100 | 0 |
| SPLEN2030371 | 0 | 0 | 0 | 0 | 0 | 100 | 0 |
| SPLEN2030397 | 0 | 0 | 0 | 0 | 0 | 100 | 0 |
| SPLEN2030479 | 0 | 0 | 0 | 0 | 0 | 100 | 0 |
| SPLEN2030495 | 0 | 0 | 0 | 0 | 0 | 100 | 0 |
| SPLEN2030562 | 0 | 0 | 0 | 0 | 0 | 100 | 0 |
| SPLEN2030763 | 0 | 74.205 | 0 | 0 | 0 | 18.320 | 0 |
| SPLEN2030847 | 0 | 0 | 0 | 0 | 0 | 100 | 0 |
| SPLEN2031004 | 0 | 0 | 0 | 0 | 0 | 100 | 0 |
| SPLEN2031125 | 0 | 0 | 0 | 0 | 0 | 100 | 0 |
| SPLEN2031424 | 0 | 0 | 0 | 0 | 0 | 13.24 | 0 |
| SPLEN2031547 | 0 | 0 | 0 | 0 | 0 | 5.478 | 0 |
| SPLEN2031674 | 0 | 0 | 0 | 0 | 0 | 38.519 | 0 |
| SPLEN2031724 | 0 | 0 | 0 | 0 | 0 | 100 | 0 |
| SPLEN2031780 | 0 | 0 | 0 | 0 | 0 | 100 | 0 |
| SPLEN2032036 | 0 | 0 | 0 | 0 | 0 | 100 | 0 |
| SPLEN2032112 | 0 | 0 | 0 | 0 | 0 | 100 | 0 |
| SPLEN2032154 | 0 | 0 | 0 | 0 | 0 | 61.070 | 0 |
| SPLEN2032157 | 0 | 0 | 0 | 0 | 0 | 9.941 | 0 |
| SPLEN2032321 | 0 | 0 | 0 | 0 | 0 | 100 | 0 |
| SPLEN2032356 | 0 | 0 | 0 | 0 | 0 | 33.427 | 0 |
| SPLEN2032677 | 0 | 0 | 0 | 0 | 0 | 100 | 0 |
| SPLEN2032813 | 0 | 0 | 0 | 0 | 0 | 100 | 0 |
| SPLEN2032924 | 0 | 0 | 0 | 0 | 0 | 100 | 0 |
| SPLEN2033098 | 0 | 0 | 0 | 0 | 0 | 25.970 | 0 |
| SPLEN2033153 | 0 | 0 | 0 | 0 | 0 | 100 | 0 |
| SPLEN2033333 | 0 | 0 | 0 | 0 | 0 | 100 | 0 |
| SPLEN2033490 | 0 | 0 | 0 | 0 | 0 | 100 | 0 |
| SPLEN2033539 | 0 | 0 | 0 | 0 | 0 | 100 | 0 |
| SPLEN2033921 | 0 | 0 | 0 | 0 | 0 | 100 | 0 |
| SPLEN2033996 | 0 | 0 | 0 | 0 | 0 | 100 | 0 |
| SPLEN2034021 | 0 | 0 | 0 | 0 | 0 | 100 | 0 |
| SPLEN2034081 | 0 | 0 | 0 | 0 | 0 | 100 | 0 |
| SPLEN2034252 | 0 | 0 | 0 | 0 | 0 | 100 | 0 |
| SPLEN2034448 | 0 | 0 | 0 | 0 | 0 | 18.039 | 0 |
| SPLEN2034551 | 0 | 0 | 0 | 0 | 0 | 100 | 0 |
| SPLEN2034601 | 0 | 0 | 0 | 0 | 0 | 100 | 0 |
| SPLEN2034678 | 0 | 0 | 0 | 0 | 0 | 100 | 0 |
| SPLEN2034781 | 0 | 0 | 0 | 0 | 0 | 48.682 | 0 |
| SPLEN2034864 | 0 | 0 | 0 | 0 | 0 | 100 | 0 |
| SPLEN2034934 | 0 | 0 | 0 | 0 | 0 | 100 | 0 |
| SPLEN2035318 | 0 | 0 | 0 | 0 | 0 | 100 | 0 |
| SPLEN2035615 | 0 | 0 | 0 | 0 | 0 | 100 | 0 |
| SPLEN2036076 | 0 | 0 | 0 | 0 | 0 | 2.858 | 0 |
| SPLEN2036103 | 0 | 0 | 0 | 0 | 0 | 100 | 0 |
| SPLEN2036326 | 0 | 0 | 0 | 0 | 0 | 100 | 0 |
| SPLEN2036475 | 0 | 0 | 0 | 0 | 0 | 100 | 0 |
| SPLEN2036501 | 0 | 0 | 0 | 0 | 0 | 67.599 | 0 |
| SPLEN2036579 | 0 | 0 | 0 | 0 | 0 | 100 | 0 |
| SPLEN2036608 | 0 | 0 | 0 | 0 | 0 | 100 | 0 |
| SPLEN2036702 | 0 | 0 | 0 | 0 | 0 | 100 | 0 |
| SPLEN2036712 | 0 | 0 | 0 | 0 | 0 | 100 | 0 |
| SPLEN2036821 | 0 | 0 | 0 | 0 | 0 | 61.070 | 0 |
| SPLEN2036932 | 0 | 0 | 0 | 0 | 0 | 15.301 | 0 |
| SPLEN2036953 | 0 | 0 | 0 | 0 | 0 | 100 | 0 |
| SPLEN2037077 | 0 | 0 | 0 | 0 | 0 | 100 | 0 |
| SPLEN2037194 | 0 | 0 | 0 | 0 | 0 | 71.023 | 0 |
| SPLEN2037319 | 0 | 0 | 0 | 0 | 0 | 100 | 0 |
| SPLEN2037444 | 0 | 0 | 0 | 0 | 0 | 100 | 0 |
| SPLEN2037580 | 0 | 0 | 0 | 0 | 0 | 100 | 0 |
| SPLEN2037630 | 0 | 0 | 0 | 0 | 0 | 100 | 0 |
| SPLEN2037678 | 0 | 0 | 0 | 0 | 0 | 100 | 0 |
| SPLEN2037722 | 0 | 0 | 0 | 0 | 0 | 14.332 | 0 |
| SPLEN2037891 | 0 | 0 | 0 | 0 | 0 | 100 | 0 |
| SPLEN2038055 | 0 | 0 | 0 | 0 | 0 | 100 | 0 |
| SPLEN2038180 | 0 | 0 | 0 | 0 | 0 | 100 | 0 |
| SPLEN2038345 | 0 | 0 | 0 | 0 | 0 | 51.152 | 0 |
| SPLEN2038407 | 0 | 0 | 0 | 0 | 0 | 100 | 0 |
| SPLEN2038473 | 0 | 0 | 0 | 0 | 0 | 23.929 | 0 |
| SPLEN2038834 | 0 | 0 | 0 | 0 | 0 | 8.059 | 0 |
| SPLEN2039311 | 0 | 0 | 0 | 0 | 0 | 100 | 0 |
| SPLEN2039379 | 0 | 0 | 0 | 0 | 0 | 5.038 | 0 |
| SPLEN2039672 | 0 | 0 | 0 | 0 | 0 | 67.599 | 0 |
| SPLEN2039697 | 0 | 0 | 0 | 0 | 0 | 100 | 0 |
| SPLEN2039845 | 0 | 0 | 0 | 0 | 0 | 100 | 0 |
| SPLEN2039936 | 0 | 0 | 0 | 0 | 0 | 100 | 0 |
| SPLEN2040222 | 0 | 0 | 0 | 0 | 0 | 100 | 0 |
| SPLEN2040237 | 0 | 0 | 0 | 0 | 0 | 71.023 | 0 |
| SPLEN2040824 | 0 | 0 | 0 | 0 | 0 | 35.879 | 0 |
| SPLEN2041304 | 0 | 0 | 0 | 0 | 0 | 100 | 0 |
| SPLEN2041306 | 0 | 0 | 0 | 0 | 0 | 26.797 | 0 |
| SPLEN2041310 | 0 | 0 | 0 | 0 | 0 | 100 | 0 |
| SPLEN2041585 | 0 | 0 | 0 | 0 | 0 | 59.326 | 0 |
| SPLEN2041645 | 0 | 0 | 0 | 0 | 0 | 100 | 0 |
| SPLEN2041700 | 0 | 0 | 0 | 0 | 0 | 100 | 0 |
| SPLEN2041720 | 0 | 0 | 0 | 0 | 0 | 100 | 0 |
| SPLEN2041977 | 0 | 0 | 0 | 0 | 0 | 100 | 0 |
| SPLEN2042051 | 0 | 0 | 0 | 0 | 0 | 100 | 0 |
| SPLEN2042303 | 0 | 0 | 0 | 0 | 0 | 45.591 | 0 |
| SPLEN2042521 | 0 | 0 | 0 | 0 | 0 | 100 | 0 |
| SPLEN2042535 | 0 | 0 | 0 | 0 | 0 | 12.386 | 0 |
| SPLEN2042598 | 0 | 0 | 0 | 0 | 0 | 100 | 0 |
| SPLEN2042714 | 0 | 0 | 0 | 0 | 0 | 59.326 | 0 |
| SPLEN2042920 | 0 | 0 | 0 | 0 | 0 | 4.734 | 0 |
| STOMA1000013 | 0 | 0 | 0 | 0 | 0 | 0 | 100 |
| STOMA1000047 | 0 | 0 | 0 | 0 | 0 | 5.512 | 21.564 |
| STOMA1000052 | 0 | 0 | 0 | 0 | 0 | 0 | 100 |
| STOMA1000074 | 0 | 0 | 0 | 0 | 0 | 0 | 100 |
| STOMA1000091 | 0 | 0 | 0 | 0 | 0 | 0 | 47.337 |
| STOMA1000117 | 0 | 0 | 0 | 0 | 0 | 0 | 100 |
| STOMA1000133 | 0 | 0 | 0 | 0 | 0 | 0 | 100 |
| STOMA1000175 | 0 | 0 | 0 | 0 | 0 | 0 | 100 |
| STOMA1000186 | 0 | 0 | 0 | 0 | 0 | 0 | 85.088 |
| STOMA1000189 | 0 | 0 | 0 | 0 | 0 | 0 | 10.642 |
| STOMA1000190 | 0 | 0 | 0 | 0 | 0 | 0 | 100 |
| STOMA1000191 | 0 | 0 | 0 | 0 | 0 | 0 | 100 |
| STOMA2000032 | 0 | 0 | 0 | 0 | 0 | 0 | 100 |
| STOMA2000072 | 0 | 0 | 0 | 0 | 0 | 0 | 100 |
| STOMA2000088 | 0 | 0 | 0 | 0 | 0 | 0 | 100 |
| STOMA2000104 | 0 | 0 | 0 | 0 | 0 | 0 | 100 |
| STOMA2000121 | 0 | 0 | 9.500 | 0 | 0 | 4.240 | 8.293 |
| STOMA2000148 | 0 | 0 | 0 | 0 | 0 | 20.359 | 79.641 |
| STOMA2000168 | 0 | 0 | 0 | 0 | 0 | 0 | 100 |
| STOMA2000183 | 0 | 0 | 0 | 0 | 0 | 0 | 100 |
| STOMA2000188 | 0 | 0 | 0 | 0 | 0 | 0 | 100 |
| STOMA2000257 | 0 | 0 | 0 | 0 | 6.966 | 0 | 13.433 |
| STOMA2000279 | 0 | 0 | 0 | 0 | 0 | 0 | 100 |
| STOMA2000289 | 0 | 0 | 0 | 0 | 9.613 | 4.739 | 18.539 |
| STOMA2000383 | 0 | 0 | 0 | 0 | 0 | 20.359 | 79.641 |
| STOMA2000386 | 0 | 0 | 0 | 0 | 0 | 0 | 100 |
| STOMA2000395 | 0 | 0 | 0 | 0 | 15.066 | 0 | 14.527 |
| STOMA2000396 | 0 | 0 | 0 | 0 | 0 | 0 | 35.071 |
| STOMA2000478 | 0 | 0 | 0 | 0 | 0 | 0 | 100 |
| STOMA2000482 | 0 | 0 | 0 | 0 | 13.604 | 0 | 26.235 |
| STOMA2000529 | 0 | 0 | 0 | 0 | 0 | 0 | 49.148 |
| STOMA2000539 | 0 | 0 | 0 | 0 | 0 | 0 | 100 |
| STOMA2000567 | 0 | 0 | 0 | 0 | 0 | 0 | 100 |
| STOMA2000640 | 0 | 0 | 0 | 0 | 0 | 0 | 100 |
| STOMA2000678 | 0 | 0 | 0 | 0 | 0 | 0 | 100 |
| STOMA2000686 | 0 | 0 | 0 | 0 | 0 | 0 | 100 |
| STOMA2000888 | 0 | 0 | 0 | 0 | 0 | 0 | 100 |
| STOMA2001025 | 0 | 0 | 0 | 0 | 0 | 0 | 100 |
| STOMA2001389 | 0 | 0 | 0 | 0 | 0 | 0 | 39.303 |
| STOMA2002052 | 0 | 0 | 0 | 0 | 0 | 0 | 37.850 |
| STOMA2002141 | 0 | 0 | 0 | 0 | 0 | 0 | 100 |
| STOMA2002166 | 0 | 4.122 | 0 | 0 | 0 | 0 | 7.963 |
| STOMA2002367 | 0 | 0 | 0 | 0 | 0 | 0 | 100 |
| STOMA2002688 | 0 | 0 | 0 | 0 | 0 | 0 | 100 |
| STOMA2002828 | 0 | 0 | 0 | 0 | 0 | 19.068 | 37.296 |
| STOMA2003158 | 0 | 0 | 0 | 0 | 0 | 53.241 | 41.655 |
| STOMA2003289 | 0 | 0 | 0 | 0 | 0 | 0 | 100 |
| STOMA2003444 | 0 | 0 | 0 | 0 | 0 | 0 | 91.236 |
| STOMA2003477 | 0 | 0 | 0 | 0 | 0 | 0 | 100 |
| STOMA2003646 | 0 | 0 | 0 | 0 | 0 | 0 | 100 |
| STOMA2003716 | 0 | 0 | 0 | 0 | 0 | 0 | 77.692 |
| STOMA2003781 | 0 | 0 | 0 | 0 | 0 | 0 | 85.866 |
| STOMA2003894 | 0 | 0 | 0 | 0 | 0 | 0 | 100 |
| STOMA2004194 | 0 | 0 | 0 | 0 | 0 | 0 | 100 |
| STOMA2004294 | 0 | 0 | 0 | 0 | 0 | 0 | 100 |
| STOMA2004663 | 0 | 0 | 0 | 0 | 0 | 0 | 47.516 |
| STOMA2004668 | 0 | 0 | 0 | 0 | 0 | 0 | 100 |
| STOMA2004852 | 0 | 0 | 0 | 0 | 0 | 0 | 100 |
| STOMA2004873 | 0 | 0 | 0 | 0 | 0 | 0 | 100 |
| STOMA2004884 | 0 | 0 | 0 | 0 | 0 | 0 | 100 |
| STOMA2004893 | 0 | 0 | 0 | 0 | 0 | 0 | 49.171 |
| STOMA2004925 | 0 | 0 | 0 | 0 | 0 | 0 | 85.088 |
| STOMA2005120 | 0 | 0 | 0 | 0 | 0 | 0 | 85.988 |
| STOMA2005664 | 0 | 0 | 0 | 0 | 0 | 0 | 100 |
| STOMA2005667 | 0 | 0 | 0 | 0 | 0 | 0 | 100 |
| STOMA2005746 | 0 | 0 | 0 | 0 | 0 | 0 | 100 |
| STOMA2005782 | 0 | 0 | 0 | 0 | 0 | 0 | 91.236 |
| STOMA2006181 | 0 | 0 | 0 | 0 | 0 | 0 | 100 |
| STOMA2006213 | 0 | 0 | 0 | 0 | 0 | 0 | 100 |
| STOMA2006229 | 0 | 0 | 0 | 0 | 0 | 0 | 40.913 |
| STOMA2006325 | 0 | 0 | 0 | 0 | 0 | 0 | 100 |
| STOMA2006330 | 0 | 0 | 0 | 0 | 0 | 0 | 100 |
| STOMA2006398 | 0 | 0 | 0 | 0 | 0 | 0 | 100 |
| STOMA2006447 | 0 | 0 | 0 | 0 | 0 | 0 | 100 |
| STOMA2006780 | 0 | 0 | 0 | 0 | 0 | 0 | 59.113 |
| STOMA2006904 | 0 | 0 | 0 | 0 | 0 | 0 | 100 |
| STOMA2007269 | 0 | 0 | 0 | 0 | 0 | 0 | 100 |
| STOMA2007680 | 0 | 0 | 0 | 0 | 0 | 0 | 100 |
| STOMA2007745 | 0 | 0 | 0 | 0 | 0 | 0 | 100 |
| STOMA2008050 | 0 | 0 | 0 | 0 | 0 | 0 | 100 |
| STOMA2008361 | 0 | 0 | 0 | 0 | 0 | 0 | 100 |
| STOMA2008546 | 0 | 0 | 0 | 0 | 0 | 0 | 100 |
| STOMA2008614 | 0 | 0 | 0 | 0 | 0 | 0 | 100 |
| STOMA2008638 | 0 | 0 | 0 | 0 | 0 | 0 | 49.866 |
| STOMA2008838 | 0 | 0 | 0 | 0 | 0 | 0 | 100 |
| STOMA2009250 | 0 | 0 | 0 | 0 | 0 | 0 | 100 |
| STOMA2009253 | 0 | 0 | 0 | 0 | 0 | 0 | 100 |
| STOMA2009256 | 0 | 0 | 0 | 0 | 0 | 0 | 100 |
| STOMA2009289 | 0 | 0 | 44.723 | 0 | 0 | 0 | 39.042 |
| SYNOV1000045 | 0 | 0 | 0 | 0 | 0 | 12.220 | 0 |
| SYNOV1000124 | 0 | 0 | 0 | 0 | 0 | 1.935 | 3.785 |
| SYNOV1000164 | 0 | 0 | 0 | 0 | 0 | 6.690 | 0 |
| SYNOV2001033 | 0 | 0 | 9.347 | 0 | 0 | 0 | 0 |
| SYNOV2001239 | 0 | 0 | 0 | 0 | 1.107 | 4.367 | 4.270 |
| SYNOV2001262 | 0 | 0 | 0 | 0 | 0 | 7.259 | 0 |
| SYNOV2001648 | 0 | 0 | 0 | 0 | 0 | 1.885 | 0 |
| SYNOV2009172 | 0 | 0 | 0 | 0 | 0 | 1.811 | 3.541 |
| SYNOV2017055 | 0 | 0 | 0 | 0 | 0 | 29.371 | 0 |
| SYNOV4006327 | 0 | 0 | 0 | 0 | 0 | 53.776 | 0 |
| SYNOV4007671 | 0 | 0 | 0 | 0 | 0 | 0 | 4.989 |
| SYNOV4009575 | 0 | 0 | 0 | 0 | 16.908 | 0 | 0 |
| TBAES2000059 | 0 | 0 | 0 | 0 | 0 | 2.621 | 0 |
| TBAES2009387 | 0 | 0 | 0 | 0 | 0 | 0.930 | 0 |
| TESOP2000527 | 0 | 0 | 0 | 0 | 0 | 17.952 | 0 |
| TESOP2006670 | 0 | 0 | 0 | 0 | 0 | 20.272 | 0 |
| TESTI1000023 | 0 | 0 | 0 | 0 | 2.959 | 1.459 | 0 |
| TESTI1000163 | 0 | 0 | 0 | 0 | 0 | 0 | 28.041 |
| TESTI2000184 | 0 | 0 | 0 | 0 | 0 | 1.966 | 0 |
| TESTI2000598 | 0 | 0 | 0 | 0 | 0 | 8.739 | 0 |
| TESTI2001099 | 0 | 0 | 0 | 0 | 1.729 | 0.852 | 0 |
| TESTI2001153 | 0 | 5.611 | 0 | 0 | 0 | 0 | 0 |
| TESTI2001339 | 0 | 0 | 0 | 0 | 0 | 72.687 | 0 |
| TESTI2001829 | 0 | 0 | 0 | 0 | 0 | 1.477 | 0 |
| TESTI2001862 | 0 | 0 | 0 | 0 | 0 | 9.200 | 0 |
| TESTI2002696 | 0 | 91.511 | 0 | 0 | 0 | 0 | 0 |
| TESTI2003454 | 0 | 0 | 0 | 0 | 18.739 | 0 | 0 |
| TESTI2003533 | 0 | 0 | 0 | 5.953 | 3.059 | 0 | 5.900 |
| TESTI2004000 | 0 | 0 | 0 | 0 | 0 | 0 | 95.417 |
| TESTI2004737 | 0 | 0 | 0 | 12.162 | 0 | 0 | 0 |
| TESTI2004929 | 0 | 60.020 | 0 | 0 | 10.019 | 0 | 0 |
| TESTI2005017 | 0 | 0 | 25.744 | 0 | 0 | 0 | 0 |
| TESTI2005408 | 0 | 0 | 0 | 0 | 0 | 3.082 | 0 |
| TESTI2005743 | 0 | 0 | 17.124 | 0 | 0 | 0 | 0 |
| TESTI2005759 | 0 | 0 | 0 | 0 | 0 | 1.627 | 0 |
| TESTI2006051 | 0 | 14.918 | 0 | 0 | 0 | 0.737 | 0 |
| TESTI2006341 | 0 | 0 | 0 | 0 | 72.130 | 0 | 0 |
| TESTI2006383 | 0 | 0 | 0 | 0 | 14.302 | 0 | 0 |
| TESTI2006588 | 0 | 3.815 | 0 | 0 | 7.642 | 3.767 | 3.684 |
| TESTI2006615 | 0 | 0 | 0 | 0 | 0 | 8.029 | 0 |
| TESTI2007407 | 0 | 0 | 23.927 | 0 | 0 | 0 | 0 |
| TESTI2007422 | 0 | 0 | 0 | 0 | 0 | 53.906 | 0 |
| TESTI2007998 | 0 | 0 | 0 | 10.857 | 0 | 0 | 0 |
| TESTI2008240 | 0 | 0 | 0 | 0 | 0.882 | 1.305 | 1.702 |
| TESTI2008699 | 0 | 0 | 0 | 0 | 0 | 0 | 66.267 |
| TESTI2009785 | 0 | 0 | 0 | 0 | 5.063 | 4.992 | 0 |
| TESTI2010513 | 0 | 0 | 0 | 0 | 1.386 | 0.683 | 0 |
| TESTI2011605 | 0 | 0 | 0 | 0 | 0 | 6.270 | 0 |
| TESTI2011712 | 0 | 0 | 0 | 0 | 72.967 | 0 | 0 |
| TESTI2012104 | 0 | 68.595 | 0 | 0 | 0 | 0 | 0 |
| TESTI2012778 | 0 | 0 | 7.202 | 2.537 | 1.304 | 0.321 | 0 |
| TESTI2013295 | 0 | 0 | 20.764 | 0 | 0 | 0 | 0 |
| TESTI2014505 | 0 | 0 | 0 | 0 | 0 | 34.281 | 0 |
| TESTI2015092 | 0 | 0 | 0 | 0 | 0 | 0 | 22.545 |
| TESTI2015626 | 0 | 0 | 5.127 | 0 | 2.321 | 1.144 | 2.238 |
| TESTI2016033 | 0 | 12.276 | 0 | 0 | 0 | 0 | 0 |
| TESTI2017954 | 0 | 0 | 5.164 | 4.549 | 2.338 | 1.152 | 0 |
| TESTI2018462 | 0 | 0 | 0 | 0 | 0 | 4.916 | 19.231 |
| TESTI2018997 | 0 | 0 | 0 | 0 | 7.349 | 3.623 | 0 |
| TESTI2020026 | 0 | 91.511 | 0 | 0 | 0 | 0 | 0 |
| TESTI2024885 | 0 | 0 | 0 | 91.307 | 0 | 0 | 0 |
| TESTI2025454 | 0 | 0 | 0 | 0 | 39.745 | 19.594 | 0 |
| TESTI2026116 | 0 | 0 | 0 | 0 | 0 | 25.947 | 0 |
| TESTI2027271 | 0 | 0 | 0 | 0 | 18.633 | 0 | 0 |
| TESTI2033441 | 0 | 0 | 0 | 0 | 3.872 | 5.727 | 0 |
| TESTI2034940 | 0 | 0 | 2.031 | 3.577 | 3.677 | 1.360 | 1.773 |
| TESTI2046456 | 0 | 0 | 0 | 0 | 1.633 | 0.805 | 0 |
| TESTI2047605 | 0 | 0 | 0 | 0 | 0 | 25.348 | 0 |
| TESTI4000014 | 0 | 0 | 1.392 | 0 | 1.890 | 0.932 | 0 |
| TESTI4001206 | 0 | 0 | 0 | 0 | 0 | 72.687 | 0 |
| TESTI4002202 | 0 | 0 | 0 | 0 | 0 | 11.535 | 0 |
| TESTI4005534 | 0 | 0 | 0 | 0 | 0 | 48.512 | 0 |
| TESTI4007816 | 0 | 0 | 0 | 0 | 0 | 7.535 | 0 |
| TESTI4009286 | 0 | 0 | 0 | 4.977 | 0 | 1.261 | 0 |
| TESTI4010851 | 0 | 0 | 0 | 3.754 | 0 | 0.951 | 3.720 |
| TESTI4013441 | 0 | 0 | 0 | 0 | 0.553 | 0.273 | 0 |
| TESTI4013474 | 0 | 0 | 0 | 0 | 3.646 | 8.987 | 0 |
| TESTI4037156 | 0 | 0 | 0 | 0 | 1.953 | 1.926 | 0 |
| TESTI4043140 | 0 | 0 | 0 | 0 | 0 | 9.647 | 0 |
| TESTI4044704 | 0 | 0 | 0 | 0 | 17.985 | 0 | 0 |
| THYMU1000010 | 0 | 0 | 0 | 0 | 0 | 14.069 | 0 |
| THYMU1000032 | 0 | 0 | 0 | 0 | 13.772 | 6.789 | 0 |
| THYMU1000083 | 0 | 0 | 0 | 0 | 10.581 | 5.216 | 0 |
| THYMU1000103 | 0 | 0 | 0 | 0 | 0 | 3.067 | 0 |
| THYMU1000109 | 0 | 0 | 0 | 0 | 0 | 0 | 5.356 |
| THYMU1000329 | 0 | 0 | 0 | 0 | 0 | 9.271 | 0 |
| THYMU1000359 | 0 | 0 | 0 | 0 | 3.783 | 26.110 | 0 |
| THYMU1000374 | 0 | 0 | 0 | 0 | 10.570 | 13.028 | 10.193 |
| THYMU1000428 | 0 | 0 | 0 | 0 | 4.012 | 1.978 | 0 |
| THYMU1000473 | 0 | 0 | 0 | 0 | 0 | 5.786 | 0 |
| THYMU1000490 | 0 | 0 | 0 | 0 | 20.235 | 0 | 0 |
| THYMU2000057 | 0 | 0 | 0 | 0 | 0 | 2.359 | 0 |
| THYMU2000120 | 0 | 0 | 0 | 0 | 0 | 67.599 | 0 |
| THYMU2000300 | 0 | 0 | 0 | 0 | 0 | 5.082 | 0 |
| THYMU2000382 | 0 | 0 | 0 | 0 | 2.139 | 6.327 | 0 |
| THYMU2000672 | 0 | 0 | 0 | 0 | 0 | 39.350 | 0 |
| THYMU2000684 | 0 | 0 | 0 | 0 | 14.692 | 0 | 0 |
| THYMU2000775 | 0 | 0 | 0 | 0 | 0 | 16.288 | 0 |
| THYMU2001195 | 0 | 0 | 0 | 0 | 0 | 67.599 | 0 |
| THYMU2001819 | 0 | 0 | 0 | 0 | 80.886 | 0 | 0 |
| THYMU2001839 | 0 | 0 | 0 | 0 | 0 | 27.218 | 0 |
| THYMU2002147 | 0 | 0 | 0 | 0 | 0 | 41.018 | 0 |
| THYMU2002583 | 0 | 0 | 0 | 0 | 1.829 | 0 | 0 |
| THYMU2002756 | 0 | 0 | 0 | 0 | 0 | 12.736 | 0 |
| THYMU2002983 | 0 | 0 | 0 | 0 | 0 | 15.619 | 0 |
| THYMU2003012 | 0 | 0 | 0 | 0 | 3.092 | 4.574 | 0 |
| THYMU2003046 | 0 | 0 | 0 | 0 | 20.350 | 10.033 | 0 |
| THYMU2003133 | 0 | 0 | 0 | 0 | 0 | 18.107 | 0 |
| THYMU2003287 | 0 | 0 | 0 | 0 | 0 | 20.684 | 0 |
| THYMU2003419 | 0 | 0 | 0 | 0 | 0 | 1.747 | 0 |
| THYMU2003446 | 0 | 0 | 0 | 0 | 8.416 | 0 | 0 |
| THYMU2003499 | 0 | 0 | 0 | 0 | 0 | 52.442 | 0 |
| THYMU2003542 | 0 | 0 | 0 | 0 | 0 | 3.760 | 0 |
| THYMU2003891 | 0 | 0 | 0 | 0 | 3.604 | 6.092 | 1.986 |
| THYMU2003932 | 0 | 0 | 0 | 0 | 0 | 33.649 | 0 |
| THYMU2003981 | 0 | 0 | 0 | 0 | 0 | 7.268 | 0 |
| THYMU2004152 | 0 | 0 | 0 | 0 | 3.504 | 13.818 | 0 |
| THYMU2004320 | 0 | 0 | 0 | 0 | 0 | 28.426 | 0 |
| THYMU2004410 | 0 | 0 | 14.936 | 0 | 0 | 9.999 | 0 |
| THYMU2004733 | 0 | 0 | 0 | 0 | 0 | 9.743 | 0 |
| THYMU2004906 | 0 | 0 | 0 | 0 | 0 | 21.804 | 0 |
| THYMU2005439 | 0 | 0 | 0 | 0 | 19.505 | 28.848 | 0 |
| THYMU2005546 | 0 | 11.562 | 0 | 0 | 0 | 0.952 | 0 |
| THYMU2005980 | 0 | 0 | 0 | 0 | 0 | 14.107 | 0 |
| THYMU2006610 | 0 | 0 | 0 | 0 | 0 | 47.241 | 0 |
| THYMU2006813 | 0 | 0 | 0 | 0 | 0 | 2.753 | 0 |
| THYMU2007060 | 0 | 0 | 0 | 0 | 0 | 7.417 | 0 |
| THYMU2007065 | 0 | 0 | 0 | 0 | 0 | 7.054 | 0 |
| THYMU2007415 | 0 | 0 | 0 | 0 | 0 | 8.419 | 0 |
| THYMU2007467 | 0 | 0 | 0 | 0 | 0 | 7.027 | 0 |
| THYMU2007694 | 0 | 0 | 0 | 0 | 0 | 36.067 | 0 |
| THYMU2007886 | 0 | 0 | 10.794 | 0 | 0 | 2.409 | 0 |
| THYMU2008321 | 0 | 0 | 0 | 0 | 0 | 5.029 | 0 |
| THYMU2008686 | 0 | 0 | 0 | 0 | 0 | 21.200 | 0 |
| THYMU2009186 | 0 | 0 | 0 | 0 | 0 | 67.599 | 0 |
| THYMU2009658 | 0 | 0 | 0 | 2.405 | 0 | 0 | 0 |
| THYMU2009792 | 0 | 0 | 0 | 0 | 0 | 4.872 | 0 |
| THYMU2010448 | 0 | 0 | 0 | 0 | 0 | 18.423 | 0 |
| THYMU2010519 | 0 | 0 | 0 | 0 | 0 | 13.829 | 0 |
| THYMU2011063 | 0 | 0 | 0 | 0 | 0 | 9.154 | 0 |
| THYMU2011482 | 0 | 0 | 0 | 0 | 10.832 | 5.340 | 0 |
| THYMU2012492 | 0 | 0 | 0 | 0 | 0 | 45.490 | 0 |
| THYMU2012690 | 0 | 0 | 0 | 0 | 0 | 6.760 | 0 |
| THYMU2013916 | 0 | 0 | 0 | 0 | 0 | 5.788 | 0 |
| THYMU2014167 | 0 | 0 | 0 | 0 | 19.391 | 0 | 0 |
| THYMU2014492 | 0 | 0 | 0 | 0 | 67.907 | 0 | 0 |
| THYMU2015762 | 0 | 0 | 0 | 6.807 | 0 | 1.725 | 0 |
| THYMU2018126 | 0 | 0 | 0 | 0 | 2.567 | 0 | 0 |
| THYMU2018639 | 0 | 0 | 0 | 0 | 0 | 67.599 | 0 |
| THYMU2024121 | 0 | 0 | 0 | 0 | 0 | 51.056 | 0 |
| THYMU2024748 | 0 | 0 | 0 | 0 | 0 | 67.599 | 0 |
| THYMU2027125 | 0 | 1.261 | 0 | 1.229 | 0 | 0.311 | 0 |
| THYMU2027975 | 0 | 0 | 0 | 0 | 0 | 6.953 | 0 |
| THYMU2028629 | 0 | 0 | 0 | 0 | 0 | 31.849 | 0 |
| THYMU2029578 | 0 | 0 | 0 | 0 | 0 | 23.924 | 0 |
| THYMU2032014 | 0 | 0 | 0 | 0 | 0 | 28.708 | 0 |
| THYMU2033263 | 0 | 78.387 | 0 | 0 | 0 | 0 | 0 |
| THYMU2034374 | 0 | 0 | 0 | 0 | 44.389 | 21.883 | 0 |
| THYMU2034647 | 0 | 0 | 0 | 0 | 0 | 67.599 | 0 |
| THYMU3003007 | 0 | 0 | 0 | 0 | 0 | 33.064 | 0 |
| THYMU3003565 | 0 | 0 | 0 | 0 | 0 | 51.056 | 0 |
| THYMU3013386 | 0 | 0 | 0 | 5.140 | 0 | 0 | 0 |
| THYMU3015759 | 0 | 0 | 8.777 | 0 | 0 | 1.959 | 0 |
| THYMU3032798 | 0 | 0 | 0 | 0 | 0 | 3.364 | 0 |
| THYMU3032867 | 0 | 0 | 0 | 0 | 0 | 22.391 | 0 |
| THYMU3040068 | 0 | 0 | 0 | 0 | 0 | 67.599 | 0 |
| THYMU3040816 | 0 | 0 | 90.337 | 0 | 0 | 0 | 0 |
| THYMU3041428 | 0 | 0 | 0 | 0 | 0 | 0.443 | 0 |
| THYMU3045510 | 0 | 0 | 0 | 0 | 0 | 41.018 | 0 |
| THYMU3047115 | 0 | 20.268 | 0 | 0 | 0 | 0 | 0 |
| TKIDN2000464 | 0 | 0 | 0 | 0 | 0 | 31.971 | 0 |
| TKIDN2002738 | 0 | 0 | 3.269 | 0 | 1.480 | 0 | 0 |
| TKIDN2005487 | 0 | 0 | 0 | 4.933 | 0 | 0 | 0 |
| TKIDN2007605 | 0 | 0 | 0 | 0 | 0 | 21.732 | 0 |
| TKIDN2011289 | 0 | 0 | 0 | 0 | 0 | 10.400 | 0 |
| TLIVE2002690 | 0 | 14.154 | 0 | 0 | 0 | 0 | 0 |
| TLIVE2003970 | 0 | 0 | 0 | 0 | 8.161 | 0 | 0 |
| TRACH1000011 | 0 | 0 | 0 | 0 | 0 | 12.805 | 0 |
| TRACH1000030 | 0 | 0 | 0 | 0 | 0 | 2.400 | 0 |
| TRACH1000038 | 0 | 0 | 0 | 0 | 26.581 | 0 | 0 |
| TRACH1000063 | 0 | 0 | 0 | 14.767 | 0 | 0 | 0 |
| TRACH1000106 | 0 | 0 | 0 | 0 | 0 | 10.328 | 0 |
| TRACH2000045 | 0 | 0 | 0 | 0 | 0 | 4.784 | 0 |
| TRACH2000047 | 0 | 0 | 0 | 0 | 0 | 9.317 | 0 |
| TRACH2000237 | 0 | 0 | 0 | 36.917 | 0 | 0 | 0 |
| TRACH2000243 | 99.654 | 0 | 0 | 0 | 0 | 0 | 0 |
| TRACH2000248 | 0 | 0 | 0 | 0 | 0 | 2.427 | 0 |
| TRACH2000282 | 0 | 0 | 0 | 0 | 0 | 60.831 | 0 |
| TRACH2000321 | 0 | 0 | 0 | 0 | 1.734 | 0 | 0 |
| TRACH2000359 | 0 | 0 | 0 | 0 | 0 | 0 | 40.588 |
| TRACH2000472 | 0 | 0 | 0 | 0 | 3.013 | 4.456 | 0 |
| TRACH2000540 | 0 | 0 | 0 | 0 | 5.027 | 4.957 | 0 |
| TRACH2000702 | 0 | 0 | 0 | 26.957 | 0 | 0 | 0 |
| TRACH2000920 | 0 | 0 | 0 | 0 | 0 | 11.535 | 0 |
| TRACH2000972 | 0 | 0 | 0 | 0 | 2.091 | 0 | 0 |
| TRACH2001021 | 0 | 2.950 | 0 | 0 | 2.955 | 1.457 | 0 |
| TRACH2001289 | 0 | 0 | 0 | 0 | 2.399 | 1.774 | 0 |
| TRACH2001428 | 0 | 0 | 0 | 0 | 0 | 2.196 | 0 |
| TRACH2001432 | 0 | 0 | 0 | 0 | 0 | 0 | 7.570 |
| TRACH2001463 | 0 | 0 | 0 | 0 | 10.804 | 10.653 | 0 |
| TRACH2001523 | 0 | 0 | 0 | 11.673 | 0 | 0 | 0 |
| TRACH2001549 | 0 | 0 | 0 | 0 | 0 | 0 | 28.775 |
| TRACH2001596 | 0 | 0 | 0 | 0 | 0 | 35.602 | 0 |
| TRACH2001607 | 0 | 0 | 0 | 6.896 | 0 | 1.747 | 0 |
| TRACH2002043 | 0 | 0 | 8.267 | 0 | 0 | 0 | 0 |
| TRACH2002138 | 0 | 0 | 0 | 0 | 75.905 | 0 | 0 |
| TRACH2002537 | 0 | 0 | 0 | 0 | 3.273 | 0 | 0 |
| TRACH2002664 | 0 | 0 | 0 | 0 | 0 | 0 | 10.356 |
| TRACH2002803 | 0 | 0 | 0 | 0 | 0 | 3.625 | 0 |
| TRACH2002847 | 0 | 0 | 35.382 | 0 | 0 | 0 | 0 |
| TRACH2003323 | 0 | 0 | 0 | 0 | 0 | 3.096 | 0 |
| TRACH2003347 | 0 | 0 | 12.385 | 0 | 0 | 0 | 0 |
| TRACH2004109 | 0 | 0 | 0 | 0 | 0 | 60.831 | 0 |
| TRACH2005811 | 0 | 0 | 8.221 | 0 | 0 | 0 | 0 |
| TRACH2006866 | 0 | 0 | 0 | 0 | 10.505 | 0 | 6.753 |
| TRACH2007399 | 0 | 0 | 0 | 0 | 1.662 | 0 | 0 |
| TRACH2007816 | 0 | 0 | 0 | 0 | 0 | 17.367 | 0 |
| TRACH2008472 | 0 | 0 | 12.231 | 0 | 0 | 0 | 0 |
| TRACH2008478 | 0 | 0 | 0 | 0 | 0 | 62.137 | 0 |
| TRACH2008540 | 0 | 0 | 0 | 0 | 0 | 82.330 | 0 |
| TRACH2008716 | 0 | 0 | 39.545 | 0 | 0 | 0 | 0 |
| TRACH2009006 | 0 | 0 | 0 | 0 | 12.384 | 0 | 0 |
| TRACH2011113 | 0 | 0 | 0 | 0 | 0 | 8.213 | 0 |
| TRACH2011500 | 0 | 0 | 0 | 0 | 0 | 23.489 | 0 |
| TRACH2012298 | 0 | 0 | 11.062 | 0 | 0 | 0 | 0 |
| TRACH2012376 | 0 | 0 | 20.922 | 0 | 0 | 0 | 0 |
| TRACH2012497 | 0 | 0 | 0 | 0 | 0 | 11.261 | 0 |
| TRACH2013495 | 0 | 0 | 87.436 | 0 | 0 | 0 | 0 |
| TRACH2014077 | 0 | 0 | 0 | 0 | 0 | 5.918 | 0 |
| TRACH2014124 | 0 | 0 | 0 | 0 | 10.927 | 7.183 | 0 |
| TRACH2015823 | 0 | 0 | 17.053 | 0 | 0 | 0 | 0 |
| TRACH2016533 | 0 | 0 | 0 | 0 | 0 | 49.869 | 0 |
| TRACH2018317 | 0 | 0 | 0 | 0 | 10.338 | 5.096 | 19.936 |
| TRACH2018950 | 0 | 0 | 0 | 0 | 0 | 0 | 51.111 |
| TRACH2019425 | 0 | 0 | 87.436 | 0 | 0 | 0 | 0 |
| TRACH2024293 | 0 | 0 | 0 | 0 | 0 | 28.081 | 0 |
| TRACH3002192 | 0 | 0 | 4.861 | 0 | 0 | 0 | 0 |
| TRACH3003547 | 0 | 0 | 0 | 0 | 0 | 7.357 | 0 |
| TRACH3005294 | 0 | 0 | 0 | 0 | 0 | 6.461 | 0 |
| TRACH3006717 | 0 | 0 | 0 | 0 | 0 | 6.383 | 0 |
| TRACH3008093 | 0 | 0 | 0 | 0 | 0 | 8.522 | 0 |
| TRACH3009008 | 0 | 0 | 38.190 | 0 | 0 | 0 | 0 |
| TRACH3037067 | 0 | 0 | 0 | 0 | 0 | 2.796 | 0 |
| TSTOM2000315 | 0 | 0 | 17.765 | 0 | 0 | 0 | 0 |
| UMVEN2000133 | 0 | 0 | 0 | 0 | 4.137 | 1.020 | 0 |
| UMVEN2000453 | 0 | 0 | 4.070 | 0 | 0 | 0 | 0 |
| UTERU2000095 | 0 | 0 | 0 | 0 | 0 | 1.440 | 0 |
| UTERU2000332 | 0 | 0 | 0 | 0 | 16.686 | 8.226 | 0 |
| UTERU2000338 | 0 | 0 | 0 | 0 | 0 | 11.607 | 0 |
| UTERU2000424 | 0 | 0 | 0 | 0 | 5.994 | 8.865 | 0 |
| UTERU2000537 | 0 | 0 | 0 | 0 | 18.105 | 8.926 | 0 |
| UTERU2000541 | 0 | 0 | 0 | 0 | 0 | 10.972 | 0 |
| UTERU2002294 | 0 | 0 | 0 | 0 | 0 | 3.158 | 0 |
| UTERU2002410 | 0 | 0 | 0 | 0 | 2.072 | 1.021 | 0 |
| UTERU2003057 | 0 | 0 | 0 | 0 | 9.127 | 0 | 0 |
| UTERU2003456 | 0 | 76.204 | 0 | 0 | 0 | 0 | 0 |
| UTERU2004197 | 0 | 0 | 0 | 0 | 7.095 | 0 | 0 |
| UTERU2004664 | 0 | 0 | 0 | 0 | 5.988 | 0 | 0 |
| UTERU2004807 | 0 | 7.079 | 0 | 0 | 3.545 | 3.495 | 0 |
| UTERU2005292 | 0 | 0 | 0 | 0 | 0 | 5.417 | 0 |
| UTERU2005354 | 0 | 0 | 0 | 0 | 0 | 3.805 | 0 |
| UTERU2005593 | 0 | 0 | 8.750 | 0 | 0 | 0 | 0 |
| UTERU2005621 | 0 | 0 | 0 | 0 | 0 | 3.921 | 0 |
| UTERU2006115 | 0 | 0 | 0 | 0 | 0 | 0 | 53.335 |
| UTERU2006593 | 0 | 0 | 11.510 | 0 | 0 | 0 | 0 |
| UTERU2008085 | 0 | 3.818 | 1.408 | 3.721 | 0.956 | 0.157 | 1.229 |
| UTERU2008302 | 0 | 0 | 10.415 | 0 | 0 | 2.324 | 0 |
| UTERU2008939 | 0 | 0 | 0 | 0 | 0 | 25.840 | 0 |
| UTERU2009538 | 0 | 0 | 0 | 0 | 0 | 59.326 | 0 |
| UTERU2010226 | 0 | 0 | 0 | 0 | 27.836 | 0 | 0 |
| UTERU2010417 | 0 | 0 | 0 | 0 | 1.113 | 0.548 | 0 |
| UTERU2012741 | 0 | 0 | 0 | 0 | 63.521 | 0 | 0 |
| UTERU2012856 | 0 | 0 | 0 | 24.224 | 0 | 0 | 0 |
| UTERU2014898 | 0 | 0 | 0 | 20.896 | 0 | 0 | 0 |
| UTERU2015190 | 0 | 0 | 0 | 0 | 0 | 59.326 | 0 |
| UTERU2016822 | 0 | 0 | 0 | 0 | 0 | 10.490 | 0 |
| UTERU2016979 | 0 | 0 | 0 | 0 | 0 | 17.993 | 0 |
| UTERU2023045 | 0 | 0 | 0 | 0 | 31.937 | 0 | 0 |
| UTERU2023550 | 0 | 59.011 | 0 | 0 | 0 | 0 | 0 |
| UTERU2031084 | 0 | 0 | 0 | 0 | 0 | 37.818 | 0 |
| UTERU3005767 | 0 | 0 | 0 | 0 | 4.200 | 2.071 | 0 |
| UTERU3015011 | 0 | 0 | 0 | 0 | 0 | 12.277 | 0 |

Genes whose expression levels were negligibly low in each of the above-described libraries are shown with their clone names below.

3NB691000018, 3NB691000085, 3NB691000113, 3NB691000129, 3NB691000191, 3NB692000012, 3NB692000029, 3NB692000102, 3NB692000154, 3NB692000276, 3NB692000305, 3NB692000374, 3NB692000429, 3NB692000484, 3NB692000529, 3NB692001034, 3NB692001040, 3NB692001123, 3NB692001267, 3NB692001288, 3NB692001349, 3NB692001366, 3NB692001408, 3NB692001433, 3NB692001471, 3NB692001496, 3NB692001501, 3NB692001507, 3NB692001511, 3NB692001519, 3NB692001520, 3NB692001548, 3NB692001557, 3NB692001637, 3NB692001719, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064,

ADIPS2000069, ADIPS2000088, ADIPS2000245, ADIPS2000425, ADRGL1000002, ADRGL1000018, ADRGL1000033, ADRGL1000038, ADRGL1000065, ADRGL1000067, ADRGL1000111, ADRGL1000147, ADRGL1000160, ADRGL1000165, ADRGL1000182, ADRGL2000006, ADRGL2000042, ADRGL2000056, ADRGL2000064, ADRGL2000074, ADRGL2000085, ADRGL2000097, ADRGL2000142, ADRGL2000172, ADRGL2000248, ADRGL2000261, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002679,

ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023,

ADRGL2008331, ADRGL2008337, ADRGL2008535, ADRGL2008966, ADRGL2009146, ADRGL2009273, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO2000011, ASTRO2000014, ASTRO2000046, ASTRO2000095, ASTRO2000141, ASTRO2000191, ASTRO2000278, ASTRO2000372, ASTRO2000381, ASTRO2000403, ASTRO2000417, ASTRO2000482, ASTRO2000533, ASTRO2000653, ASTRO2000662, ASTRO2000725, ASTRO2000801, ASTRO2001001, ASTRO2001008, ASTRO2001029, ASTRO2001088, ASTRO2001107, ASTRO2001122, ASTRO2001227, ASTRO2001480,

ASTRO2002024, ASTRO2002035, ASTRO2002064, ASTRO2002202, ASTRO2002459, ASTRO2002571, ASTRO2002632, ASTRO2002659, ASTRO2002693, ASTRO2002720, ASTRO2002733, ASTRO2002743, ASTRO2002842, ASTRO2003212, ASTRO2003316, ASTRO2003461, ASTRO2003574, ASTRO2003581, ASTRO2003632, ASTRO2003740, ASTRO2003840, ASTRO2003925, ASTRO2003960, ASTRO2004032, ASTRO2004051, ASTRO2004177, ASTRO2004217, ASTRO2004297, ASTRO2004584, ASTRO2004628, ASTRO2004751, ASTRO2004877, ASTRO2004974, ASTRO2005008, ASTRO2005081, ASTRO2005096, ASTRO2005242, ASTRO2005273, ASTRO2005343, ASTRO2005413, ASTRO2005423, ASTRO2005557, ASTRO2005575, ASTRO2005593, ASTRO2005726, ASTRO2005863, ASTRO2005902, ASTRO2006356, ASTRO2006475, ASTRO2006732,

ASTRO2006747, ASTRO2006920, ASTRO2006952, ASTRO2007221, ASTRO2007377, ASTRO2007515, ASTRO2007578, ASTRO2007643, ASTRO2007666, ASTRO2007948, ASTRO2007956, ASTRO2007962, ASTRO2008040, ASTRO2008216, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392,

ASTRO2014498, ASTRO2014561, ASTRO2014576, ASTRO2014863, ASTRO2014923, ASTRO2014961, ASTRO2015162, ASTRO2015185, ASTRO2015328, ASTRO2015529, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018169, ASTRO2018373, ASTRO2019039, ASTRO2019171, ASTRO3000154, ASTRO3000172, ASTRO3000177, ASTRO3000301, ASTRO3000474, ASTRO3000482, BEAST2000454, BGGI11000193, BGGI11000285, BGGI12000022, BGGI12000067, BGGI12000161, BGGI12000533, BGGI12000544, BGGI12000684, BGGI12000693, BGGI12001002, BGGI12001097, BGGI12001241, BGGI12001247, BGGI12001682, BLADE1000176, BLADE2000181, BLADE2000340, BLADE2000389, BLADE2000463,

BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2005036, BLADE2005038, BLADE2005229, BLADE2005459, BLADE2005792, BLADE2006043, BLADE2006412, BLADE2006415, BLADE2006607, BLADE2006826, BLADE2006830, BLADE2006849, BLADE2006947, BLADE2007033, BLADE2007043, BLADE2007589, BLADE2007666, BLADE2007722,

BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000153, BNGH41000169, BNGH41000177, BNGH41000198, BNGH41000216, BNGH42000130, BNGH42000132, BNGH42000405, BNGH42000532, BNGH42000675, BNGH42000791, BNGH42000815, BNGH42000994, BNGH42001247, BNGH42001406, BNGH42001724, BNGH42002244, BNGH42002387, BNGH42002487, BNGH42002489, BNGH42003529, BNGH42003570, BNGH42003641, BNGH42004076, BNGH42004291, BNGH42004538, BNGH42004679, BNGH42005017, BNGH42005235, BNGH42005504,

BNGH42005968, BNGH42006135, BNGH42006226, BNGH42006234, BNGH42007037, BNGH42007460, BNGH42007594, BNGH42007788, BNGH42007798, BNGH42008199, BNGH42008510, BNGH42008603, BNGH42008649, BNGH42008743, BNGH42008832, BNGH42008850, BNGH42008943, BNGH42009021, BRACE1000020, BRACE1000047, BRACE1000065, BRACE1000070, BRACE1000073, BRACE1000084, BRACE1000087, BRACE1000092, BRACE1000093, BRACE1000101, BRACE1000114, BRACE1000124, BRACE1000132, BRACE1000159, BRACE1000166, BRACE1000169, BRACE1000186, BRACE1000187, BRACE1000239, BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229,

BRACE2000232, BRACE2000245, BRACE2000256, BRACE2000266, BRACE2000280, BRACE2000300, BRACE2000331, BRACE2000351, BRACE2000368, BRACE2000392, BRACE2000441, BRACE2000487, BRACE2000489, BRACE2000505, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000750, BRACE2000815, BRACE2000885, BRACE2000893, BRACE2000905, BRACE2000936, BRACE2000947, BRACE2000965, BRACE2000969, BRACE2000975, BRACE2000988, BRACE2001017, BRACE2001065, BRACE2001070, BRACE2001094, BRACE2001107, BRACE2001117, BRACE2001143, BRACE2001154, BRACE2001188, BRACE2001191, BRACE2001197, BRACE2001236,

BRACE2001239, BRACE2001269, BRACE2001312, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001497, BRACE2001508, BRACE2001534, BRACE2001543, BRACE2001564, BRACE2001588, BRACE2001607, BRACE2001626, BRACE2001677, BRACE2001692, BRACE2001705, BRACE2001711, BRACE2001726, BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001892, BRACE2001898, BRACE2001923, BRACE2001942, BRACE2001944, BRACE2001954, BRACE2001971, BRACE2001972, BRACE2001973,

BRACE2002026, BRACE2002034, BRACE2002081, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002392, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002649, BRACE2002676, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002812,

BRACE2002834, BRACE2002849, BRACE2002857, BRACE2002861, BRACE2002884, BRACE2002896, BRACE2002948, BRACE2003037, BRACE2003078, BRACE2003097, BRACE2003110, BRACE2003168, BRACE2003199, BRACE2003210, BRACE2003217, BRACE2003269, BRACE2003285, BRACE2003304, BRACE2003398, BRACE2003420, BRACE2003428, BRACE2003431, BRACE2003512, BRACE2003516, BRACE2003527, BRACE2003539, BRACE2003540, BRACE2003594, BRACE2003598, BRACE2003609, BRACE2003614, BRACE2003628, BRACE2003639, BRACE2003700, BRACE2003713, BRACE2003766, BRACE2003785, BRACE2003788, BRACE2003800, BRACE2003802, BRACE2003825, BRACE2003847, BRACE2003848, BRACE2003873, BRACE2003885, BRACE2003887, BRACE2003905, BRACE2003928, BRACE2003982, BRACE2003987,

BRACE2005034, BRACE2005047, BRACE2005063, BRACE2005083, BRACE2005090, BRACE2005099, BRACE2005118, BRACE2005124, BRACE2005138, BRACE2005151, BRACE2005160, BRACE2005169, BRACE2005189, BRACE2005193, BRACE2005196, BRACE2005198, BRACE2005216, BRACE2005231, BRACE2005243, BRACE2005251, BRACE2005253, BRACE2005290, BRACE2005328, BRACE2005329, BRACE2005337, BRACE2005348, BRACE2005363, BRACE2005395, BRACE2005408, BRACE2005433, BRACE2005448, BRACE2005450, BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787,

BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006072, BRACE2006075, BRACE2006084, BRACE2006089, BRACE2006102, BRACE2006105, BRACE2006122, BRACE2006137, BRACE2006143, BRACE2006167, BRACE2006174, BRACE2006240, BRACE2006245, BRACE2006249, BRACE2006274, BRACE2006281, BRACE2006298, BRACE2006319, BRACE2006344, BRACE2006354, BRACE2006378, BRACE2006380, BRACE2006393, BRACE2006413, BRACE2006417, BRACE2006453, BRACE2006488, BRACE2006534, BRACE2006540, BRACE2006547, BRACE2006564, BRACE2006587, BRACE2006636, BRACE2006685, BRACE2006703,

BRACE2006743, BRACE2006753, BRACE2006783, BRACE2006821, BRACE2006833, BRACE2006859, BRACE2006871, BRACE2006909, BRACE2006911, BRACE2006924, BRACE2006935, BRACE2006937, BRACE2006944, BRACE2006951, BRACE2006982, BRACE2006986, BRACE2007013, BRACE2007087, BRACE2007138, BRACE2007174, BRACE2007197, BRACE2007201, BRACE2007203, BRACE2007232, BRACE2007262, BRACE2007274, BRACE2007281, BRACE2007358, BRACE2007396, BRACE2007398, BRACE2007401, BRACE2007411, BRACE2007447, BRACE2007477, BRACE2007502, BRACE2007503, BRACE2007522, BRACE2007527, BRACE2007538, BRACE2007563, BRACE2007577, BRACE2007621, BRACE2007640, BRACE2007641, BRACE2007646, BRACE2007663, BRACE2007682, BRACE2007685, BRACE2007727, BRACE2007749,

BRACE2007760, BRACE2007761, BRACE2007764, BRACE2007768, BRACE2007769, BRACE2007784, BRACE2007786, BRACE2007798, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008097, BRACE2008102, BRACE2008114, BRACE2008144, BRACE2008172, BRACE2008218, BRACE2008251, BRACE2008276, BRACE2008295, BRACE2008308, BRACE2008336, BRACE2008361, BRACE2008380, BRACE2008399, BRACE2008401, BRACE2008402, BRACE2008410, BRACE2008420, BRACE2008443, BRACE2008473, BRACE2008480, BRACE2008481, BRACE2008488, BRACE2008495, BRACE2008500, BRACE2008594, BRACE2008604,

BRACE2008615, BRACE2008622, BRACE2008629, BRACE2008655, BRACE2008656, BRACE2008706, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008941, BRACE2008948, BRACE2008960, BRACE2009014, BRACE2009016, BRACE2009031, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009291, BRACE2009293, BRACE2009307, BRACE2009311, BRACE2009318, BRACE2009323, BRACE2009421, BRACE2009428, BRACE2009434, BRACE2009437, BRACE2009483,

BRACE2009533, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011747,

BRACE2011838, BRACE2011892, BRACE2011904, BRACE2011947, BRACE2012062, BRACE2012185, BRACE2012222, BRACE2012232, BRACE2012291, BRACE2012317, BRACE2012510, BRACE2012625, BRACE2012653, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2014108, BRACE2014161, BRACE2014232, BRACE2014257, BRACE2014268, BRACE2014306, BRACE2014475, BRACE2014657, BRACE2014746, BRACE2014780, BRACE2014821, BRACE2014824, BRACE2014850, BRACE2014854, BRACE2015031, BRACE2015058, BRACE2015132, BRACE2015262, BRACE2015270, BRACE2015314, BRACE2015352, BRACE2015368, BRACE2015412, BRACE2015436, BRACE2015741,

BRACE2015756, BRACE2015995, BRACE2016058, BRACE2016194, BRACE2016315, BRACE2016325, BRACE2016335, BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017397, BRACE2017410, BRACE2017438, BRACE2017574, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019258, BRACE2019348, BRACE2019467,

BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021014, BRACE2021148, BRACE2021245, BRACE2021254, BRACE2021264, BRACE2021344, BRACE2021433, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022394, BRACE2022435, BRACE2022448, BRACE2022450, BRACE2022549, BRACE2022633, BRACE2022693, BRACE2022848,

BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026920, BRACE2026971, BRACE2027018, BRACE2027221, BRACE2027258, BRACE2027312, BRACE2027382, BRACE2027389, BRACE2027408, BRACE2027643, BRACE2027767,

BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030341, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786,

BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138,

BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492,

BRACE2047558, BRACE2047573, BRACE2047835, BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004150, BRACE3004205, BRACE3004358, BRACE3004371, BRACE3004435, BRACE3004477,

BRACE3004767, BRACE3004772, BRACE3004783, BRACE3004843, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384,

BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010438, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013936, BRACE3013986, BRACE3014005,

BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805,

BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447,

BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856,

BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834,

BRALZ2002174, BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168,

BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000277, BRAMY2000388, BRAMY2000411, BRAMY2000508, BRAMY2000562, BRAMY2000585, BRAMY2000653, BRAMY2000814, BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311,

BRAMY2002339, BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004363, BRAMY2004387, BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182,

BRAMY2005244, BRAMY2005391, BRAMY2005488, BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105,

BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162,

BRAMY2012163, BRAMY2012277, BRAMY2012340, BRAMY2012497, BRAMY2012517, BRAMY2012536, BRAMY2012555, BRAMY2012568, BRAMY2012581, BRAMY2012691, BRAMY2012721, BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015855, BRAMY2015890,

BRAMY2015911, BRAMY2015925, BRAMY2016002, BRAMY2016009, BRAMY2016020, BRAMY2016070, BRAMY2016251, BRAMY2016325, BRAMY2016327, BRAMY2016386, BRAMY2016706, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019600, BRAMY2019643, BRAMY2019710, BRAMY2019963, BRAMY2019985, BRAMY2019989, BRAMY2020050, BRAMY2020058, BRAMY2020106,

BRAMY2020270, BRAMY2020354, BRAMY2020355, BRAMY2020412, BRAMY2020445, BRAMY2020466, BRAMY2020520, BRAMY2020574, BRAMY2020634, BRAMY2020713, BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238,

BRAMY2023327, BRAMY2023358, BRAMY2023406, BRAMY2023482, BRAMY2023514, BRAMY2023729, BRAMY2023786, BRAMY2023852, BRAMY2023863, BRAMY2023939, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538,

BRAMY2026685, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028740, BRAMY2028783, BRAMY2028796, BRAMY2028856, BRAMY2028914, BRAMY2029204, BRAMY2029602, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859,

BRAMY2030915, BRAMY2031317, BRAMY2031377, BRAMY2031442, BRAMY2031516, BRAMY2032014, BRAMY2032087, BRAMY2032231, BRAMY2032242, BRAMY2032290, BRAMY2032311, BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396,

BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244,

BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409,

BRAMY3001794, BRAMY3002120, BRAMY3002312, BRAMY3002329, BRAMY3002458, BRAMY3002508, BRAMY3002620, BRAMY3002803, BRAMY3002805, BRAMY3002886, BRAMY3002991, BRAMY3003026, BRAMY3003109, BRAMY3003205, BRAMY3003310, BRAMY3003566, BRAMY3003723, BRAMY3004126, BRAMY3004224, BRAMY3004364, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158,

BRAMY3009207, BRAMY3009491, BRAMY3009556, BRAMY3009782, BRAMY3009811, BRAMY3009904, BRAMY3010264, BRAMY3010321, BRAMY3010411, BRAMY3010492, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRASW1000053, BRASW1000125, BRAWH1000001, BRAWH1000002, BRAWH1000007, BRAWH1000037, BRAWH1000045, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131,

BRAWH2000142, BRAWH2000169, BRAWH2000218, BRAWH2000232, BRAWH2000248, BRAWH2000256, BRAWH2000274, BRAWH2000323, BRAWH2000353, BRAWH2000370, BRAWH2000409, BRAWH2000443, BRAWH2000471, BRAWH2000476, BRAWH2000482, BRAWH2000488, BRAWH2000494, BRAWH2000500, BRAWH2000503, BRAWH2000554, BRAWH2000595, BRAWH2000636, BRAWH2000643, BRAWH2000651, BRAWH2000682, BRAWH2000686, BRAWH2000697, BRAWH2000752, BRAWH2000859, BRAWH2000866, BRAWH2000892, BRAWH2000901, BRAWH2000926, BRAWH2000984, BRAWH2001019, BRAWH2001037, BRAWH2001052, BRAWH2001129, BRAWH2001137, BRAWH2001159, BRAWH2001171, BRAWH2001203, BRAWH2001238, BRAWH2001239, BRAWH2001255, BRAWH2001355, BRAWH2001378, BRAWH2001395, BRAWH2001406, BRAWH2001412,

BRAWH2001418, BRAWH2001424, BRAWH2001436, BRAWH2001438, BRAWH2001439, BRAWH2001459, BRAWH2001461, BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002333, BRAWH2002352, BRAWH2002383, BRAWH2002549, BRAWH2002560, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003135,

BRAWH2003240, BRAWH2003307, BRAWH2003355, BRAWH2003366, BRAWH2003662, BRAWH2003689, BRAWH2003693, BRAWH2003832, BRAWH2003948, BRAWH2003964, BRAWH2004044, BRAWH2004068, BRAWH2004078, BRAWH2004136, BRAWH2004217, BRAWH2004731, BRAWH2004779, BRAWH2004842, BRAWH2004884, BRAWH2004923, BRAWH2005008, BRAWH2005068, BRAWH2005074, BRAWH2005225, BRAWH2005315, BRAWH2005517, BRAWH2005524, BRAWH2005533, BRAWH2005578, BRAWH2005812, BRAWH2005974, BRAWH2005998, BRAWH2006044, BRAWH2006083, BRAWH2006207, BRAWH2006301, BRAWH2006395, BRAWH2006450, BRAWH2006493, BRAWH2006526, BRAWH2006543, BRAWH2006622, BRAWH2006760, BRAWH2006960, BRAWH2006989, BRAWH2007069, BRAWH2007308, BRAWH2007406, BRAWH2007550, BRAWH2007570,

BRAWH2007591, BRAWH2007658, BRAWH2007664, BRAWH2007808, BRAWH2007825, BRAWH2007862, BRAWH2007890, BRAWH2008058, BRAWH2008255, BRAWH2008297, BRAWH2008366, BRAWH2008380, BRAWH2008540, BRAWH2008577, BRAWH2008706, BRAWH2008790, BRAWH2008903, BRAWH2008956, BRAWH2008993, BRAWH2009112, BRAWH2009227, BRAWH2009238, BRAWH2009261, BRAWH2009263, BRAWH2009298, BRAWH2009307, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364, BRAWH2010536, BRAWH2010552, BRAWH2010584, BRAWH2010618, BRAWH2010619, BRAWH2010754,

BRAWH2010980, BRAWH2011066, BRAWH2011079, BRAWH2011096, BRAWH2011155, BRAWH2011166, BRAWH2011204, BRAWH2011294, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014234, BRAWH2014379, BRAWH2014414, BRAWH2014473, BRAWH2014511, BRAWH2014662, BRAWH2014717,

BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016160, BRAWH2016221, BRAWH2016223, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017542, BRAWH2017635, BRAWH2017913, BRAWH2018206, BRAWH2018267, BRAWH2018282, BRAWH2018317, BRAWH2018506, BRAWH2018514, BRAWH2018601, BRAWH2018745,

BRAWH2018875, BRAWH2019053, BRAWH2019055, BRAWH2019198, BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002513, BRAWH3002574, BRAWH3002600, BRAWH3002819, BRAWH3003019, BRAWH3003136, BRAWH3003244, BRAWH3003343, BRAWH3003411, BRAWH3003522, BRAWH3003573, BRAWH3003598, BRAWH3003727, BRAWH3003801, BRAWH3003975, BRAWH3004222, BRAWH3004335, BRAWH3004350, BRAWH3004453, BRAWH3004530, BRAWH3004666,

BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011637, BRAWH3011685, BRAWH3011907, BRAWH3011929, BRAWH3012005,

BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021545, BRAWH3021574, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900,

BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3029806, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117,

BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000168, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923,

BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007426, BRCAN2007525, BRCAN2008494, BRCAN2008528, BRCAN2008701, BRCAN2008894, BRCAN2009156, BRCAN2009168,

BRCAN2009203, BRCAN2009432, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016762, BRCAN2016814, BRCAN2017442, BRCAN2017905, BRCAN2018240, BRCAN2018269, BRCAN2018490, BRCAN2018667, BRCAN2018748, BRCAN2018935, BRCAN2019002,

BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368, BRCAN2025712, BRCAN2025982, BRCAN2026117, BRCAN2026197, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028040, BRCAN2028319, BRCAN2028338, BRCAN2028355, BRCAN2028378, BRCAN2028460,

BRCAN2028545, BRCAN2028634, BRCAN2028702, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942, BRCOC2007034,

BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010123, BRCOC2010510, BRCOC2010980, BRCOC2010994, BRCOC2011010, BRCOC2011398, BRCOC2011418, BRCOC2011506, BRCOC2011769, BRCOC2011996, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015944, BRCOC2016525, BRCOC2016661, BRCOC2017061, BRCOC2017652, BRCOC2017856, BRCOC2018775, BRCOC2019255, BRCOC2019549, BRCOC2019841, BRCOC2019896, BRCOC2019903,

BRCOC2019910, BRCOC2019934, BRCOC2020122, BRCOC2020142, BRHIP1000058, BRHIP1000072, BRHIP1000108, BRHIP1000129, BRHIP1000174, BRHIP1000258, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000553, BRHIP2000621, BRHIP2000691, BRHIP2000826, BRHIP2000920, BRHIP2001074, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828,

BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007305, BRHIP2007307, BRHIP2007616, BRHIP2007690, BRHIP2007741, BRHIP2007928, BRHIP2007969, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120,

BRHIP2011199, BRHIP2011508, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014228, BRHIP2014285, BRHIP2014291, BRHIP2014373, BRHIP2014386, BRHIP2014387, BRHIP2014954, BRHIP2015224, BRHIP2015245, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385, BRHIP2017404, BRHIP2017542,

BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018719, BRHIP2018805, BRHIP2018998, BRHIP2019007, BRHIP2019047, BRHIP2019149, BRHIP2019177, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020842, BRHIP2020859, BRHIP2020930, BRHIP2021102, BRHIP2021289, BRHIP2021495, BRHIP2021615, BRHIP2021744, BRHIP2021858, BRHIP2021870, BRHIP2021929, BRHIP2022006, BRHIP2022054, BRHIP2022221, BRHIP2022228, BRHIP2022298, BRHIP2022326, BRHIP2022467, BRHIP2022708, BRHIP2022986,

BRHIP2023071, BRHIP2023229, BRHIP2023272, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024549, BRHIP2024789, BRHIP2024911, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283,

BRHIP2029393, BRHIP2029529, BRHIP2029643, BRHIP2029663, BRHIP3000060, BRHIP3000070, BRHIP3000081, BRHIP3000131, BRHIP3000240, BRHIP3000377, BRHIP3000457, BRHIP3000475, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004416, BRHIP3004710, BRHIP3004725, BRHIP3004774, BRHIP3004786,

BRHIP3004863, BRHIP3004968, BRHIP3005037, BRHIP3005137, BRHIP3005142, BRHIP3005231, BRHIP3005307, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007223, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427,

BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3012997, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3017855, BRHIP3018278, BRHIP3018784, BRHIP3019643, BRHIP3019824, BRHIP3019880, BRHIP3019956, BRHIP3020046, BRHIP3020155, BRHIP3020182,

BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000498, BRSSN2000518, BRSSN2000561, BRSSN2000566, BRSSN2000684, BRSSN2000715,

BRSSN2000832, BRSSN2001213, BRSSN2001275, BRSSN2001342, BRSSN2001426, BRSSN2001503, BRSSN2001579, BRSSN2001758, BRSSN2001810, BRSSN2001869, BRSSN2002857, BRSSN2002859, BRSSN2003086, BRSSN2003093, BRSSN2003820, BRSSN2003841, BRSSN2003937, BRSSN2004142, BRSSN2004303, BRSSN2004304, BRSSN2004496, BRSSN2004657, BRSSN2004686, BRSSN2004710, BRSSN2004719, BRSSN2005847, BRSSN2006133, BRSSN2006390, BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009378, BRSSN2009389, BRSSN2009395, BRSSN2009627, BRSSN2009630, BRSSN2009702,

BRSSN2010019, BRSSN2010110, BRSSN2010587, BRSSN2010596, BRSSN2010830, BRSSN2011262, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014424, BRSSN2014513, BRSSN2014556, BRSSN2014573, BRSSN2014610, BRSSN2014636, BRSSN2014685, BRSSN2015199, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757,

BRSSN2018218, BRSSN2018440, BRSSN2018447, BRSSN2018581, BRSSN2018731, BRSSN2018771, BRSSN2018925, BRSTN1000097, BRSTN2000058, BRSTN2000070, BRSTN2000220, BRSTN2000312, BRSTN2000367, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416,

BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451, BRSTN2013354, BRSTN2013502, BRSTN2013931, BRSTN2014490, BRSTN2014633, BRSTN2014751, BRSTN2014770, BRSTN2015015, BRSTN2015889, BRSTN2015978, BRSTN2016066, BRSTN2016335, BRSTN2016678, BRSTN2016892, BRSTN2016918, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969,

BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005942, BRTHA2005956, BRTHA2006075, BRTHA2006146, BRTHA2006194, BRTHA2006232, BRTHA2006720,

BRTHA2006735, BRTHA2006974, BRTHA2006975, BRTHA2007060, BRTHA2007122, BRTHA2007422, BRTHA2007603, BRTHA2007665, BRTHA2007998, BRTHA2008316, BRTHA2008335, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562,

BRTHA2012714, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016267, BRTHA2016318, BRTHA2016496, BRTHA2016543, BRTHA2016552, BRTHA2016577, BRTHA2016765, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420,

BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026290, BRTHA2026311, BRTHA2027227, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033155, BRTHA2033264,

BRTHA2033320, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003074, BRTHA3003225, BRTHA3003339, BRTHA3003343, BRTHA3003417, BRTHA3003449, BRTHA3003474, BRTHA3003490, BRTHA3003704, BRTHA3004307, BRTHA3004432, BRTHA3004475, BRTHA3004502, BRTHA3004610, BRTHA3005046, BRTHA3005735,

BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011229, BRTHA3011265, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547,

BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020314, BRTHA3020369, BRTHA3021569, BRTHA3021708, BRTHA3021786, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505,

BRTHA3028782, CD34C2000051, CD34C2000152, CD34C2000175, CD34C2000443, CD34C3000125, CD34C3000252, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, COLON1000030, COLON1000084, COLON1000135, COLON2000470, COLON2000557, COLON2000568, COLON2001721, COLON2001829, COLON2001866, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733,

COLON2008105, COLON2009337, COLON2009499, CORDB1000140, CORDB2000061, CORDB2000127, CORDB2000541, CTONG1000009, CTONG1000020, CTONG1000022, CTONG1000040, CTONG1000056, CTONG1000062, CTONG1000086, CTONG1000087, CTONG1000088, CTONG1000093, CTONG1000097, CTONG1000113, CTONG1000137, CTONG1000165, CTONG1000175, CTONG1000180, CTONG1000181, CTONG1000218, CTONG1000220, CTONG1000277, CTONG1000283, CTONG1000288, CTONG1000302, CTONG1000467, CTONG1000476, CTONG1000488, CTONG1000508, CTONG1000540, CTONG2000031, CTONG2000034, CTONG2000042, CTONG2000201, CTONG2000218, CTONG2000279, CTONG2000303, CTONG2000330, CTONG2000395, CTONG2000411, CTONG2000465, CTONG2000469, CTONG2000480, CTONG2000561, CTONG2000589,

CTONG2000766, CTONG2000771, CTONG2000819, CTONG2000827, CTONG2000846, CTONG2000855, CTONG2000977, CTONG2001139, CTONG2001320, CTONG2001366, CTONG2001413, CTONG2001428, CTONG2001513, CTONG2001524, CTONG2001533, CTONG2001598, CTONG2001681, CTONG2001724, CTONG2001748, CTONG2001749, CTONG2001800, CTONG2001820, CTONG2001858, CTONG2001877, CTONG2001911, CTONG2001955, CTONG2002066, CTONG2002073, CTONG2002095, CTONG2002143, CTONG2002395, CTONG2002417, CTONG2002418, CTONG2002453, CTONG2002520, CTONG2002590, CTONG2002709, CTONG2002721, CTONG2002766, CTONG2002803, CTONG2002816, CTONG2002820, CTONG2002832, CTONG2002841, CTONG2002903, CTONG2002965, CTONG2002999, CTONG2003028, CTONG2003094, CTONG2003100,

CTONG2003115, CTONG2003189, CTONG2003245, CTONG2003293, CTONG2003348, CTONG2003350, CTONG2003372, CTONG2003375, CTONG2003427, CTONG2003517, CTONG2003524, CTONG2003599, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004423, CTONG2004454, CTONG2004487, CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005278, CTONG2005290, CTONG2005374, CTONG2005399, CTONG2005468, CTONG2005567, CTONG2005585, CTONG2005615, CTONG2005635, CTONG2005775, CTONG2005795, CTONG2005890,

CTONG2005904, CTONG2005913, CTONG2006129, CTONG2006223, CTONG2006235, CTONG2006273, CTONG2006310, CTONG2006377, CTONG2006393, CTONG2006412, CTONG2006449, CTONG2006515, CTONG2006524, CTONG2006568, CTONG2006620, CTONG2006666, CTONG2006691, CTONG2006798, CTONG2006836, CTONG2006942, CTONG2006964, CTONG2007009, CTONG2007072, CTONG2007091, CTONG2007104, CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008269, CTONG2008321,

CTONG2008343, CTONG2008398, CTONG2008402, CTONG2008405, CTONG2008466, CTONG2008518, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958,

CTONG2009963, CTONG2010018, CTONG2010024, CTONG2010116, CTONG2010133, CTONG2010148, CTONG2010276, CTONG2010330, CTONG2010508, CTONG2010566, CTONG2010623, CTONG2010649, CTONG2010652, CTONG2010803, CTONG2010821, CTONG2011188, CTONG2011429, CTONG2011474, CTONG2011676, CTONG2011765, CTONG2011779, CTONG2011801, CTONG2011815, CTONG2011822, CTONG2011825, CTONG2011920, CTONG2011985, CTONG2012001, CTONG2012029, CTONG2012050, CTONG2012077, CTONG2012101, CTONG2012123, CTONG2012158, CTONG2012230, CTONG2012401, CTONG2012422, CTONG2012447, CTONG2012453, CTONG2012473, CTONG2012476, CTONG2012533, CTONG2012554, CTONG2012564, CTONG2012607, CTONG2012661, CTONG2012724, CTONG2012738, CTONG2012745, CTONG2012758,

CTONG2012843, CTONG2012879, CTONG2012901, CTONG2012996, CTONG2013128, CTONG2013149, CTONG2013156, CTONG2013178, CTONG2013222, CTONG2013284, CTONG2013339, CTONG2013352, CTONG2013381, CTONG2013393, CTONG2013511, CTONG2013630, CTONG2013803, CTONG2013934, CTONG2013986, CTONG2014032, CTONG2014058, CTONG2014119, CTONG2014191, CTONG2014206, CTONG2014234, CTONG2014264, CTONG2014280, CTONG2014369, CTONG2014491, CTONG2014630, CTONG2014635, CTONG2014646, CTONG2014705, CTONG2014898, CTONG2014946, CTONG2014959, CTONG2014966, CTONG2014995, CTONG2015204, CTONG2015316, CTONG2015330, CTONG2015345, CTONG2015358, CTONG2015434, CTONG2015518, CTONG2015540, CTONG2015596, CTONG2015633, CTONG2015678, CTONG2015717,

CTONG2015804, CTONG2015866, CTONG2015914, CTONG2015953, CTONG2016056, CTONG2016217, CTONG2016355, CTONG2016408, CTONG2016499, CTONG2016505, CTONG2016559, CTONG2016658, CTONG2016775, CTONG2016797, CTONG2016824, CTONG2016846, CTONG2016904, CTONG2016931, CTONG2016942, CTONG2016953, CTONG2017021, CTONG2017094, CTONG2017292, CTONG2017409, CTONG2017429, CTONG2017458, CTONG2017500, CTONG2017604, CTONG2017650, CTONG2017798, CTONG2017804, CTONG2017814, CTONG2017910, CTONG2017939, CTONG2017989, CTONG2018062, CTONG2018069, CTONG2018147, CTONG2018217, CTONG2018279, CTONG2018334, CTONG2018343, CTONG2018379, CTONG2018413, CTONG2018483, CTONG2018629, CTONG2018632, CTONG2018652, CTONG2018653, CTONG2018655,

CTONG2018735, CTONG2018739, CTONG2018808, CTONG2018843, CTONG2018900, CTONG2018976, CTONG2019029, CTONG2019063, CTONG2019144, CTONG2019232, CTONG2019248, CTONG2019400, CTONG2019590, CTONG2019652, CTONG2019704, CTONG2019788, CTONG2019822, CTONG2019833, CTONG2020031, CTONG2020108, CTONG2020127, CTONG2020171, CTONG2020374, CTONG2020378, CTONG2020411, CTONG2020445, CTONG2020484, CTONG2020522, CTONG2020560, CTONG2020582, CTONG2020638, CTONG2020806, CTONG2020831, CTONG2020974, CTONG2021024, CTONG2021132, CTONG2021168, CTONG2022153, CTONG2022601, CTONG2022835, CTONG2023021, CTONG2023449, CTONG2023512, CTONG2024031, CTONG2024206, CTONG2024614, CTONG2024749, CTONG2025496, CTONG2025513, CTONG2025516,

CTONG2025610, CTONG2025900, CTONG2026513, CTONG2026770, CTONG2026920, CTONG2026987, CTONG2027150, CTONG2027263, CTONG2027327, CTONG2027361, CTONG2027591, CTONG2027736, CTONG2027783, CTONG2027828, CTONG2027959, CTONG2028097, CTONG2028208, CTONG2028309, CTONG2028486, CTONG2028518, CTONG2028687, CTONG2028758, CTONG3000657, CTONG3000686, CTONG3000707, CTONG3000896, CTONG3001123, CTONG3001420, CTONG3001501, CTONG3001560, CTONG3001605, CTONG3001746, CTONG3002020, CTONG3002127, CTONG3002370, CTONG3002412, CTONG3002433, CTONG3002518, CTONG3002552, CTONG3002588, CTONG3002674, CTONG3002728, CTONG3002835, CTONG3002909, CTONG3002947, CTONG3002983, CTONG3003165, CTONG3003179, CTONG3003483, CTONG3003553,

CTONG3003598, CTONG3003652, CTONG3003654, CTONG3003669, CTONG3003737, CTONG3003905, CTONG3004072, CTONG3004317, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009028, CTONG3009227, CTONG3009287, CTONG3009328, CTONG3009385, D3OST1000066, D3OST1000109, D3OST1000238, D3OST1000267, D3OST2000184,

D3OST2000618, D3OST2000654, D3OST2000734, D3OST2001328, D3OST2001669, D3OST2001670, D3OST2001788, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000258, D3OST3000291, D3OST3000388, D3OST3000493, D6OST2000127, D6OST2000169, D6OST2000245, D6OST2000358, D6OST2000464, D6OST2000507, D9OST2000031, D9OST2000278, D9OST2000440, D9OST2000869, D9OST2001319, D9OST2001433, D9OST2002397, D9OST2002673, D9OST2003106, D9OST2003108, D9OST2003137, D9OST2003397, D9OST2003580, D9OST2003594, D9OST2003762, D9OST2003791, D9OST2003989, D9OST2004018, D9OST2004073,

D9OST2004417, DFNES1000003, DFNES1000107, DFNES1000185, DFNES2000011, DFNES2000143, DFNES2000146, DFNES2000212, DFNES2000268, DFNES2000292, DFNES2000335, DFNES2000426, DFNES2000432, DFNES2000457, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2002966, DFNES2003192, DFNES2003255, DFNES2003742, DFNES2004346, DFNES2004354, DFNES2004371, DFNES2004383, DFNES2004608, DFNES2004684, DFNES2005266, DFNES2005527, DFNES2005540, DFNES2005690, DFNES2005779, DFNES2005875, DFNES2006944, DFNES2007113,

DFNES2007299, DFNES2007332, DFNES2007641, DFNES2007757, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000023, FCBBF1000024, FCBBF1000025, FCBBF1000038, FCBBF1000053, FCBBF1000061, FCBBF1000063, FCBBF1000069, FCBBF1000115, FCBBF1000117, FCBBF1000118, FCBBF1000121, FCBBF1000125, FCBBF1000131, FCBBF1000139, FCBBF1000155, FCBBF1000157, FCBBF1000171, FCBBF1000174, FCBBF1000182, FCBBF1000198, FCBBF1000206, FCBBF1000220, FCBBF1000232, FCBBF1000233, FCBBF1000243, FCBBF1000270, FCBBF1000272, FCBBF1000280, FCBBF1000297, FCBBF1000305,

FCBBF1000314, FCBBF1000322, FCBBF1000349, FCBBF1000374, FCBBF1000376, FCBBF1000377, FCBBF1000395, FCBBF1000397, FCBBF1000425, FCBBF1000449, FCBBF1000476, FCBBF1000506, FCBBF1000509, FCBBF1000546, FCBBF1000550, FCBBF1000556, FCBBF1000563, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000748, FCBBF2000038, FCBBF2000087, FCBBF2000094, FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566, FCBBF2000576, FCBBF2000677, FCBBF2000678,

FCBBF2000685, FCBBF2000733, FCBBF2000782, FCBBF2000808, FCBBF2000815, FCBBF2000825, FCBBF2000885, FCBBF2000940, FCBBF2000951, FCBBF2001001, FCBBF2001088, FCBBF2001105, FCBBF2001116, FCBBF2001183, FCBBF2001188, FCBBF2001238, FCBBF2001243, FCBBF2001299, FCBBF2001309, FCBBF2001400, FCBBF2001427, FCBBF2001480, FCBBF2001529, FCBBF2001538, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002349, FCBBF2002370, FCBBF2002541, FCBBF2002626, FCBBF2002897, FCBBF2002898, FCBBF2002904, FCBBF2002953, FCBBF2003083, FCBBF2003102, FCBBF2003269, FCBBF2003293, FCBBF2003297, FCBBF2003395, FCBBF2003528, FCBBF2003543,

FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005909, FCBBF2005966, FCBBF2006131, FCBBF2006380, FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000120, FCBBF3000168, FCBBF3000175, FCBBF3000228, FCBBF3000229, FCBBF3000233, FCBBF3000269, FCBBF3000361, FCBBF3000367, FCBBF3000434,

FCBBF3000462, FCBBF3000473, FCBBF3000501, FCBBF3000518, FCBBF3000536, FCBBF3000550, FCBBF3000847, FCBBF3001018, FCBBF3001020, FCBBF3001081, FCBBF3001235, FCBBF3001281, FCBBF3001302, FCBBF3001377, FCBBF3001594, FCBBF3001632, FCBBF3001657, FCBBF3001818, FCBBF3001855, FCBBF3001890, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004261, FCBBF3004323, FCBBF3004390,

FCBBF3004409, FCBBF3004502, FCBBF3004715, FCBBF3004842, FCBBF3004847, FCBBF3004955, FCBBF3005160, FCBBF3005218, FCBBF3005444, FCBBF3005698, FCBBF3005729, FCBBF3006171, FCBBF3006249, FCBBF3006288, FCBBF3006399, FCBBF3006628, FCBBF3006821, FCBBF3007077, FCBBF3007150, FCBBF3007152, FCBBF3007242, FCBBF3007244, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008159, FCBBF3008251, FCBBF3008311, FCBBF3008362, FCBBF3008382, FCBBF3008475, FCBBF3008556, FCBBF3008644, FCBBF3008689, FCBBF3008748, FCBBF3008870, FCBBF3008944, FCBBF3009069, FCBBF3009101, FCBBF3009152,

FCBBF3009256, FCBBF3009317, FCBBF3009428, FCBBF3009464, FCBBF3009526, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012347, FCBBF3012443, FCBBF3012588, FCBBF3012667, FCBBF3012842, FCBBF3012901, FCBBF3013041, FCBBF3013058, FCBBF3013205, FCBBF3013266, FCBBF3013307, FCBBF3013341,

FCBBF3013506, FCBBF3013623, FCBBF3013800, FCBBF3013846, FCBBF3014229, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016987, FCBBF3017024, FCBBF3017059, FCBBF3017071, FCBBF3017123, FCBBF3017233, FCBBF3017255, FCBBF3017288, FCBBF3017396, FCBBF3017531, FCBBF3017535, FCBBF3017681, FCBBF3017729, FCBBF3017873, FCBBF3017918, FCBBF3017974, FCBBF3018067, FCBBF3018173, FCBBF3018453, FCBBF3018591, FCBBF3018753, FCBBF3018826, FCBBF3018949, FCBBF3019085, FCBBF3019320, FCBBF3019437,

FCBBF3019455, FCBBF3019564, FCBBF3019569, FCBBF3019570, FCBBF3019663, FCBBF3019716, FCBBF3019784, FCBBF3019839, FCBBF3019867, FCBBF3019961, FCBBF3020030, FCBBF3020138, FCBBF3020140, FCBBF3020163, FCBBF3020232, FCBBF3021026, FCBBF3021191, FCBBF3021221, FCBBF3021501, FCBBF3021506, FCBBF3021524, FCBBF3021807, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078, FCBBF3023368, FCBBF3023372, FCBBF3023443, FCBBF3023599, FCBBF3023704, FCBBF3023887, FCBBF3023895, FCBBF3024002, FCBBF3024096, FCBBF3024225, FCBBF3024266, FCBBF3024364, FCBBF3024623, FCBBF3024793, FCBBF3024911,

FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3025737, FCBBF3025905, FCBBF3026021, FCBBF3026048, FCBBF3026251, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028244, FCBBF3028472, FCBBF3028528, FCBBF3028582, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000529,

FCBBF4000548, FCBBF5000041, FCBBF5000061, FCBBF5000165, FCBBF5000261, FCBBF5000325, FCBBF5000353, FCBBF5000384, FCBBF5000483, FCBBF5000495, FEBRA1000005, FEBRA1000008, FEBRA1000030, FEBRA1000057, FEBRA1000088, FEBRA1000138, FEBRA1000148, FEBRA1000149, FEBRA1000183, FEBRA1000188, FEBRA1000189, FEBRA2000007, FEBRA2000021, FEBRA2000035, FEBRA2000053, FEBRA2000126, FEBRA2000220, FEBRA2000253, FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000454, FEBRA2000462, FEBRA2000468, FEBRA2000491,

FEBRA2000504, FEBRA2000532, FEBRA2000536, FEBRA2000538, FEBRA2000575, FEBRA2000581, FEBRA2000656, FEBRA2000659, FEBRA2000680, FEBRA2000690, FEBRA2000727, FEBRA2000734, FEBRA2000757, FEBRA2000771, FEBRA2000772, FEBRA2000782, FEBRA2000790, FEBRA2000793, FEBRA2000803, FEBRA2000805, FEBRA2000809, FEBRA2000815, FEBRA2000856, FEBRA2000862, FEBRA2000877, FEBRA2000881, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001245, FEBRA2001267, FEBRA2001334, FEBRA2001351,

FEBRA2001383, FEBRA2001438, FEBRA2001440, FEBRA2001487, FEBRA2001561, FEBRA2001584, FEBRA2001590, FEBRA2001591, FEBRA2001610, FEBRA2001620, FEBRA2001659, FEBRA2001698, FEBRA2001705, FEBRA2001706, FEBRA2001715, FEBRA2001745, FEBRA2001752, FEBRA2001772, FEBRA2001790, FEBRA2001814, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002399, FEBRA2002410, FEBRA2002429, FEBRA2002442, FEBRA2002456, FEBRA2002508, FEBRA2002525, FEBRA2002527, FEBRA2002552, FEBRA2002574, FEBRA2002601, FEBRA2002611, FEBRA2002628, FEBRA2002662, FEBRA2002682, FEBRA2002707, FEBRA2002727, FEBRA2002781, FEBRA2002783,

FEBRA2002792, FEBRA2002818, FEBRA2002858, FEBRA2002882, FEBRA2002897, FEBRA2002908, FEBRA2002931, FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003275, FEBRA2003308, FEBRA2003327, FEBRA2003429, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003822, FEBRA2003833, FEBRA2003897, FEBRA2003907, FEBRA2004023, FEBRA2004026, FEBRA2004029, FEBRA2004053, FEBRA2004056, FEBRA2004091, FEBRA2004110, FEBRA2004191, FEBRA2004202, FEBRA2004219, FEBRA2004237, FEBRA2004268, FEBRA2004325, FEBRA2004329, FEBRA2004331,

FEBRA2004443, FEBRA2004485, FEBRA2004490, FEBRA2004538, FEBRA2004592, FEBRA2004620, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005416, FEBRA2005427, FEBRA2005458, FEBRA2005476, FEBRA2005701, FEBRA2005726, FEBRA2005752, FEBRA2005787, FEBRA2005995, FEBRA2005998, FEBRA2006055, FEBRA2006061, FEBRA2006112, FEBRA2006150, FEBRA2006165, FEBRA2006255, FEBRA2006315, FEBRA2006354, FEBRA2006357, FEBRA2006368, FEBRA2006370, FEBRA2006372, FEBRA2006379, FEBRA2006396, FEBRA2006409,

FEBRA2006418, FEBRA2006427, FEBRA2006476, FEBRA2006485, FEBRA2006519, FEBRA2006521, FEBRA2006553, FEBRA2006627, FEBRA2006664, FEBRA2006667, FEBRA2006726, FEBRA2006793, FEBRA2006808, FEBRA2006817, FEBRA2006873, FEBRA2006890, FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007620, FEBRA2007708, FEBRA2007714, FEBRA2007793, FEBRA2007818, FEBRA2007823, FEBRA2007880, FEBRA2007900, FEBRA2007931, FEBRA2007957, FEBRA2008009, FEBRA2008036, FEBRA2008086, FEBRA2008134, FEBRA2008135, FEBRA2008159,

FEBRA2008165, FEBRA2008172, FEBRA2008255, FEBRA2008265, FEBRA2008266, FEBRA2008282, FEBRA2008287, FEBRA2008311, FEBRA2008360, FEBRA2008459, FEBRA2008468, FEBRA2008481, FEBRA2008542, FEBRA2008660, FEBRA2008662, FEBRA2008681, FEBRA2008692, FEBRA2008741, FEBRA2008836, FEBRA2008859, FEBRA2008861, FEBRA2008866, FEBRA2008881, FEBRA2008928, FEBRA2008983, FEBRA2009016, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009419, FEBRA2009437, FEBRA2009478, FEBRA2009507, FEBRA2009514, FEBRA2009567, FEBRA2009588, FEBRA2009731,

FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572,

FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2018746, FEBRA2019172, FEBRA2019242, FEBRA2019298, FEBRA2019389, FEBRA2019457, FEBRA2019519, FEBRA2019582, FEBRA2019637, FEBRA2019663, FEBRA2019690, FEBRA2019867, FEBRA2020406, FEBRA2020566, FEBRA2020582, FEBRA2020668, FEBRA2020801, FEBRA2020845, FEBRA2020886, FEBRA2021032, FEBRA2021134, FEBRA2021171, FEBRA2021339, FEBRA2021431, FEBRA2021497, FEBRA2021550, FEBRA2021571, FEBRA2021864,

FEBRA2021892, FEBRA2021908, FEBRA2022013, FEBRA2022148, FEBRA2022204, FEBRA2022255, FEBRA2022322, FEBRA2022504, FEBRA2022601, FEBRA2022696, FEBRA2022729, FEBRA2022787, FEBRA2022911, FEBRA2022956, FEBRA2022963, FEBRA2022972, FEBRA2023285, FEBRA2023351, FEBRA2023377, FEBRA2023399, FEBRA2023498, FEBRA2023550, FEBRA2023721, FEBRA2023927, FEBRA2023989, FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025463, FEBRA2025477, FEBRA2025838, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297,

FEBRA2027352, FEBRA2027364, FEBRA2027609, FEBRA2028158, FEBRA2028222, FEBRA2028366, FEBRA2028457, FEBRA2028477, FEBRA2028503, FEBRA2028516, FEHRT2000325, FEKID1000018, FELIV1000101, FELIV1000137, FELIV1000164, FELNG2000241, HCASM1000021, HCASM1000050, HCASM1000061, HCASM1000115, HCASM1000130, HCASM2000016, HCASM2000138, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005388, HCASM2005484, HCASM2006131,

HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000142, HCHON1000176, HCHON1000191, HCHON2000038, HCHON2000062, HCHON2000100, HCHON2000156, HCHON2000199, HCHON2000212, HCHON2000265, HCHON2000271, HCHON2000315, HCHON2000322, HCHON2000344, HCHON2000364, HCHON2000395, HCHON2000417, HCHON2000418, HCHON2000473, HCHON2000503, HCHON2000508, HCHON2000660, HCHON2000663, HCHON2000673, HCHON2000676, HCHON2000698, HCHON2000699, HCHON2000705, HCHON2000743, HCHON2000815, HCHON2000817, HCHON2000818, HCHON2000819, HCHON2000832, HCHON2000851, HCHON2000898, HCHON2000935,

HCHON2000942, HCHON2000956, HCHON2000994, HCHON2001050, HCHON2001084, HCHON2001099, HCHON2001108, HCHON2001110, HCHON2001116, HCHON2001161, HCHON2001214, HCHON2001233, HCHON2001269, HCHON2001434, HCHON2001450, HCHON2001453, HCHON2001497, HCHON2001519, HCHON2001523, HCHON2001535, HCHON2001546, HCHON2001548, HCHON2001577, HCHON2001598, HCHON2001604, HCHON2001607, HCHON2001646, HCHON2001665, HCHON2001712, HCHON2001768, HCHON2001801, HCHON2001853, HCHON2002123, HCHON2002194, HCHON2002241, HCHON2002247, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003659, HCHON2003676, HCHON2004007,

HCHON2004279, HCHON2004359, HCHON2004531, HCHON2004776, HCHON2004858, HCHON2005048, HCHON2005118, HCHON2005166, HCHON2005802, HCHON2005921, HCHON2005987, HCHON2006459, HCHON2006714, HCHON2006722, HCHON2006770, HCHON2006786, HCHON2006841, HCHON2006871, HCHON2007046, HCHON2007482, HCHON2007674, HCHON2007774, HCHON2007881, HCHON2008112, HCHON2008239, HCHON2008444, HCHON2008582, HCHON2008672, HCHON2008856, HCHON2008871, HCHON2008989, HCHON2009191, HCHON2009384, HCHON2009749, HCHON2009766, HCHON2009795, HCHON2010074, HCHON2010543, HEART1000088, HEART1000099, HEART1000102, HEART1000118, HEART1000139, HEART1000142, HEART1000151, HEART1000173, HEART2000024, HEART2000035, HEART2000099, HEART2000298,

HEART2000306, HEART2000309, HEART2000411, HEART2000418, HEART2000448, HEART2000492, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2004570, HEART2004764, HEART2004931, HEART2004940, HEART2004941, HEART2004980, HEART2005244, HEART2005310, HEART2005581, HEART2005610, HEART2006131, HEART2006310, HEART2006334, HEART2006487, HEART2006557, HEART2006787, HEART2006789, HEART2006909, HEART2007031, HEART2007231, HEART2007443, HEART2007696, HEART2007767, HEART2008108, HEART2008111, HEART2008257,

HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC1000114, HHDPC1000118, HHDPC2000070, HHDPC2000102, HHDPC2000104, HHDPC2000258, HHDPC2000315, HHDPC2000455, HHDPC2000456, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005742, HHDPC2006862, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114,

HHDPC2009178, HHDPC2009208, HHDPC2009272, HHDPC2009528, HLUNG1000017, HLUNG1000024, HLUNG1000030, HLUNG1000034, HLUNG1000053, HLUNG1000076, HLUNG1000084, HLUNG1000091, HLUNG1000099, HLUNG1000104, HLUNG1000105, HLUNG1000110, HLUNG1000151, HLUNG1000169, HLUNG2000004, HLUNG2000014, HLUNG2000027, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000314, HLUNG2000315, HLUNG2000340, HLUNG2000362, HLUNG2000363, HLUNG2000431, HLUNG2000480, HLUNG2000489, HLUNG2000549, HLUNG2000557, HLUNG2000570, HLUNG2000751, HLUNG2000761, HLUNG2000777, HLUNG2000846, HLUNG2000870, HLUNG2000884, HLUNG2000926,

HLUNG2000955, HLUNG2000970, HLUNG2001013, HLUNG2001126, HLUNG2001144, HLUNG2001146, HLUNG2001214, HLUNG2001459, HLUNG2001507, HLUNG2001633, HLUNG2001677, HLUNG2001712, HLUNG2001814, HLUNG2001996, HLUNG2002006, HLUNG2002050, HLUNG2002059, HLUNG2002085, HLUNG2002295, HLUNG2002303, HLUNG2002405, HLUNG2002429, HLUNG2002562, HLUNG2002659, HLUNG2002707, HLUNG2002811, HLUNG2002916, HLUNG2002942, HLUNG2002958, HLUNG2002982, HLUNG2003049, HLUNG2003061, HLUNG2003162, HLUNG2003246, HLUNG2003306, HLUNG2003335, HLUNG2003378, HLUNG2003497, HLUNG2003691, HLUNG2003710, HLUNG2003714, HLUNG2003716, HLUNG2003833, HLUNG2003872, HLUNG2003918, HLUNG2003991, HLUNG2004014, HLUNG2004037, HLUNG2004067, HLUNG2004154,

HLUNG2004170, HLUNG2004217, HLUNG2004273, HLUNG2004416, HLUNG2004521, HLUNG2004534, HLUNG2004684, HLUNG2004707, HLUNG2004774, HLUNG2004775, HLUNG2005012, HLUNG2005030, HLUNG2005045, HLUNG2005076, HLUNG2005133, HLUNG2005203, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614, HLUNG2006671, HLUNG2006781, HLUNG2006852, HLUNG2006935, HLUNG2007041, HLUNG2007210, HLUNG2007219, HLUNG2007245, HLUNG2007433, HLUNG2007931, HLUNG2008066, HLUNG2008139, HLUNG2008153, HLUNG2008235, HLUNG2008247, HLUNG2008333, HLUNG2008348, HLUNG2008384, HLUNG2008396,

HLUNG2008439, HLUNG2008479, HLUNG2008480, HLUNG2008521, HLUNG2008833, HLUNG2008875, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017262, HLUNG2017286, HLUNG2017307,

HLUNG2017350, HLUNG2017546, HLUNG2017806, HLUNG2018029, HLUNG2018282, HLUNG2018626, HLUNG2019058, HSYRA1000131, HSYRA1000148, HSYRA1000152, HSYRA2000044, HSYRA2000135, HSYRA2000137, HSYRA2000221, HSYRA2000237, HSYRA2000253, HSYRA2000371, HSYRA2000424, HSYRA2000507, HSYRA2000510, HSYRA2000605, HSYRA2000629, HSYRA2000706, HSYRA2000760, HSYRA2000787, HSYRA2000792, HSYRA2000828, HSYRA2000832, HSYRA2000927, HSYRA2001003, HSYRA2001105, HSYRA2001142, HSYRA2001153, HSYRA2001232, HSYRA2001255, HSYRA2001332, HSYRA2001353, HSYRA2001484, HSYRA2001552, HSYRA2001580, HSYRA2001615, HSYRA2001621, HSYRA2001631, HSYRA2001638, HSYRA2002103, HSYRA2002590, HSYRA2002741, HSYRA2003168, HSYRA2004550, HSYRA2004858,

HSYRA2005456, HSYRA2005496, HSYRA2005628, HSYRA2005970, HSYRA2006049, HSYRA2006873, HSYRA2007241, HSYRA2007338, HSYRA2007667, HSYRA2008154, HSYRA2008714, HSYRA2009102, IMR321000155, IMR321000161, IMR321000165, IMR321000193, IMR321000207, IMR321000230, IMR321000266, IMR322000010, IMR322000107, IMR322000164, IMR322000379, IMR322000383, IMR322000418, IMR322000518, IMR322000555, IMR322000604, IMR322000609, IMR322000656, IMR322000672, IMR322000775, IMR322000791, IMR322000811, IMR322000819, IMR322000838, IMR322000859, IMR322000863, IMR322000917, IMR322000918, IMR322000919, IMR322000953, IMR322000984, IMR322001018, IMR322001167, IMR322001185, IMR322001245, IMR322001318, IMR322001322, IMR322001332,

IMR322001380, IMR322001452, IMR322001491, IMR322001534, IMR322001665, IMR322001670, IMR322001710, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, IMR322019070, KIDNE1000008, KIDNE1000012, KIDNE1000015, KIDNE1000028, KIDNE1000036, KIDNE1000064,

KIDNE1000104, KIDNE1000121, KIDNE1000143, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000287, KIDNE2000315, KIDNE2000317, KIDNE2000328, KIDNE2000330, KIDNE2000335, KIDNE2000349, KIDNE2000375, KIDNE2000394, KIDNE2000403, KIDNE2000422, KIDNE2000493, KIDNE2000510, KIDNE2000513, KIDNE2000517, KIDNE2000519, KIDNE2000555, KIDNE2000624, KIDNE2000665, KIDNE2000678, KIDNE2000717, KIDNE2000721, KIDNE2000722, KIDNE2000801, KIDNE2000832, KIDNE2000846, KIDNE2000867, KIDNE2000947, KIDNE2001117, KIDNE2001140, KIDNE2001269, KIDNE2001361, KIDNE2001636, KIDNE2001713, KIDNE2001802,

KIDNE2001840, KIDNE2001847, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002262, KIDNE2002265, KIDNE2002438, KIDNE2002483, KIDNE2002725, KIDNE2002768, KIDNE2002795, KIDNE2002798, KIDNE2002839, KIDNE2002845, KIDNE2002882, KIDNE2002980, KIDNE2002991, KIDNE2003185, KIDNE2003188, KIDNE2003233, KIDNE2003316, KIDNE2003335, KIDNE2003357, KIDNE2003373, KIDNE2003377, KIDNE2003752, KIDNE2003764, KIDNE2003837, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305, KIDNE2004344, KIDNE2004394, KIDNE2004411,

KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005676, KIDNE2005781, KIDNE2005798, KIDNE2005854, KIDNE2005908, KIDNE2005937, KIDNE2006030, KIDNE2006053, KIDNE2006062, KIDNE2006092, KIDNE2006145, KIDNE2006149, KIDNE2006210, KIDNE2006299, KIDNE2006334, KIDNE2006376, KIDNE2006378, KIDNE2006465, KIDNE2006580, KIDNE2006652, KIDNE2006687,

KIDNE2006733, KIDNE2006760, KIDNE2006775, KIDNE2006820, KIDNE2006880, KIDNE2007040, KIDNE2007077, KIDNE2007222, KIDNE2007356, KIDNE2007422, KIDNE2007569, KIDNE2007810, KIDNE2007944, KIDNE2007954, KIDNE2008022, KIDNE2008069, KIDNE2008072, KIDNE2008116, KIDNE2008117, KIDNE2008218, KIDNE2008315, KIDNE2008362, KIDNE2008378, KIDNE2008403, KIDNE2008404, KIDNE2008446, KIDNE2008473, KIDNE2008480, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2009019, KIDNE2009109, KIDNE2009180, KIDNE2009191, KIDNE2009332, KIDNE2009367, KIDNE2009426, KIDNE2009467, KIDNE2009490, KIDNE2009553, KIDNE2009605, KIDNE2009628,

KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012188, KIDNE2012291, KIDNE2012316, KIDNE2012361, KIDNE2012440, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013346,

KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014170, KIDNE2014184, KIDNE2014268, KIDNE2014298, KIDNE2014320, KIDNE2014325, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539,

KIDNE2016570, KIDNE2016918, KIDNE2017007, KIDNE2017040, KIDNE2017052, KIDNE2017151, KIDNE2017153, KIDNE2017315, KIDNE2017332, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018462, KIDNE2018617, KIDNE2018678, KIDNE2018727, KIDNE2018863, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000079, LIVER1000082, LIVER1000099, LIVER1000104, LIVER1000111, LIVER1000126, LIVER1000139, LIVER1000155, LIVER1000165, LIVER1000175, LIVER1000224, LIVER1000230, LIVER1000278,

LIVER1000303, LIVER1000397, LIVER1000433, LIVER1000479, LIVER1000482, LIVER1000542, LIVER2000003, LIVER2000033, LIVER2000037, LIVER2000158, LIVER2000216, LIVER2000237, LIVER2000247, LIVER2000360, LIVER2000416, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001539, LIVER2001608, LIVER2001835, LIVER2002638, LIVER2002644, LIVER2002660, LIVER2002702, LIVER2003008, LIVER2003065, LIVER2003234, LIVER2003511, LIVER2003524, LIVER2003568, LIVER2003581, LIVER2003800, LIVER2003854, LIVER2004074, LIVER2004392,

LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005973, LIVER2005981, LIVER2006006, LIVER2006251, LIVER2006410, LIVER2006469, LIVER2006644, LIVER2006764, LIVER2007415, LIVER2007548, LIVER2007568, LIVER2007721, LIVER2008053, LIVER2008465, LIVER2008473, LIVER2008580, LIVER2008706, LIVER2008945, LIVER2008998, LIVER2009110, LIVER2009118, LIVER2009554, LYMPB1000141, LYMPB1000158, LYMPB2000083, MAMGL1000017, MAMGL1000083, MAMGL1000096, MAMGL1000100, MAMGL1000132, MAMGL1000151, MAMGL1000182, MAMGL1000184, MESAN1000032, MESAN1000035, MESAN1000050, MESAN1000079,

MESAN1000080, MESAN1000101, MESAN1000121, MESAN1000147, MESAN1000180, MESAN2000023, MESAN2000026, MESAN2000067, MESAN2000092, MESAN2000167, MESAN2000291, MESAN2000337, MESAN2000434, MESAN2000457, MESAN2000462, MESAN2000572, MESAN2000620, MESAN2000711, MESAN2000815, MESAN2000894, MESAN2000909, MESAN2001450, MESAN2001627, MESAN2001770, MESAN2001979, MESAN2001980, MESAN2002113, MESAN2002122, MESAN2002147, MESAN2002186, MESAN2002424, MESAN2002519, MESAN2002687, MESAN2002709, MESAN2002724, MESAN2002790, MESAN2002844, MESAN2002978, MESAN2003035, MESAN2003037, MESAN2003039, MESAN2003058, MESAN2003101, MESAN2003190, MESAN2003322, MESAN2003490, MESAN2003529, MESAN2003622, MESAN2003646, MESAN2003662,

MESAN2003709, MESAN2003851, MESAN2004138, MESAN2004575, MESAN2005303, MESAN2005371, MESAN2005633, MESAN2005724, MESAN2005766, MESAN2005808, MESAN2005811, MESAN2005957, MESAN2006022, MESAN2006043, MESAN2006328, MESAN2006401, MESAN2006580, MESAN2006599, MESAN2006743, MESAN2006953, MESAN2006971, MESAN2007032, MESAN2007440, MESAN2007597, MESAN2007755, MESAN2008222, MESAN2008334, MESAN2008415, MESAN2008460, MESAN2008536, MESAN2008679, MESAN2008729, MESAN2008821, MESAN2008926, MESAN2008936, MESAN2009019, MESAN2009105, MESAN2009311, MESAN2009418, MESAN2009503, MESAN2009522, MESAN2010031, MESAN2010114, MESAN2010312, MESAN2010321, MESAN2010664, MESAN2010681, MESAN2011545, MESAN2011597, MESAN2011627,

MESAN2011632, MESAN2011977, MESAN2011984, MESAN2012054, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013908, MESAN2013936, MESAN2014192, MESAN2014295, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016552, MESAN2016965, MESAN2017133, MESAN2017152, MESAN2017373, MESAN2017417, MESAN2018209, MESAN2018576, MESAN2018670, MESAN2018699, MESTC1000042, MESTC1000147, MESTC2000150, MESTC2000153, MESTC2000170, N1ESE2000698, NB9N41000011,

NB9N41000047, NB9N41000121, NB9N41000142, NB9N41000146, NB9N41000340, NB9N42000104, NB9N42000122, NB9N42000196, NB9N42000314, NB9N42000342, NB9N42000430, NB9N42000495, NB9N42001300, NCRRP1000129, NESOP1000010, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000187, NHNPC2000212, NHNPC2000606, NHNPC2000877, NHNPC2001108, NHNPC2001223, NHNPC2001312, NHNPC2001816, NHNPC2001931,

NHNPC2002565, NHNPC2002749, NOVAR1000091, NOVAR1000102, NOVAR2000136, NOVAR2000352, NOVAR2000412, NOVAR2000710, NOVAR2000783, NOVAR2000962, NOVAR2001108, NOVAR2001620, NOVAR2001783, NOVAR2001876, NOVAR2002039, NT2NE1000004, NT2NE1000014, NT2NE1000018, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000195, NT2NE2000222, NT2NE2000284, NT2NE2000299, NT2NE2000327, NT2NE2000353, NT2NE2000356, NT2NE2000369, NT2NE2000374, NT2NE2000384, NT2NE2000386, NT2NE2000390, NT2NE2000392, NT2NE2000455,

NT2NE2000488, NT2NE2000517, NT2NE2000536, NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000809, NT2NE2000864, NT2NE2000909, NT2NE2000913, NT2NE2000951, NT2NE2000980, NT2NE2001005, NT2NE2001021, NT2NE2001040, NT2NE2001048, NT2NE2001142, NT2NE2001176, NT2NE2001180, NT2NE2001188, NT2NE2001191, NT2NE2001247, NT2NE2001252, NT2NE2001324, NT2NE2001326, NT2NE2001337, NT2NE2001364, NT2NE2001372, NT2NE2001397, NT2NE2001403, NT2NE2001428, NT2NE2001432, NT2NE2001435, NT2NE2001442, NT2NE2001524, NT2NE2001530, NT2NE2001545, NT2NE2001598, NT2NE2001623, NT2NE2001626, NT2NE2001634, NT2NE2001648, NT2NE2001655,

NT2NE2001660, NT2NE2001666, NT2NE2001677, NT2NE2001697, NT2NE2001698, NT2NE2001723, NT2NE2001725, NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150, NT2NE2003252, NT2NE2003280, NT2NE2003308, NT2NE2003309, NT2NE2003315, NT2NE2003393, NT2NE2003408, NT2NE2003457, NT2NE2003540, NT2NE2003569, NT2NE2003571, NT2NE2003705, NT2NE2003752, NT2NE2003887, NT2NE2003921, NT2NE2004255, NT2NE2004378, NT2NE2004490, NT2NE2004606, NT2NE2004716, NT2NE2004740, NT2NE2004787, NT2NE2004916, NT2NE2005035, NT2NE2005223, NT2NE2005276, NT2NE2005317,

NT2NE2005323, NT2NE2005358, NT2NE2005395, NT2NE2005419, NT2NE2005517, NT2NE2005537, NT2NE2005555, NT2NE2005588, NT2NE2005676, NT2NE2005720, NT2NE2005784, NT2NE2005804, NT2NE2005821, NT2NE2005890, NT2NE2005921, NT2NE2005968, NT2NE2006087, NT2NE2006103, NT2NE2006217, NT2NE2006288, NT2NE2006382, NT2NE2006387, NT2NE2006427, NT2NE2006432, NT2NE2006478, NT2NE2006531, NT2NE2006659, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727,

NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010842, NT2NE2010854, NT2NE2011033, NT2NE2011036, NT2NE2011107, NT2NE2011119, NT2NE2011154, NT2NE2011221, NT2NE2011411, NT2NE2011485, NT2NE2011650, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012847,

NT2NE2012928, NT2NE2013019, NT2NE2013081, NT2NE2013189, NT2NE2013217, NT2NE2013547, NT2NE2014013, NT2NE2014028, NT2NE2014104, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721,

NT2NE2017752, NT2NE2017792, NT2NE2017982, NT2NE2018165, NT2NE2018176, NT2NE2018273, NT2NE2018376, NT2NE2018472, NT2NE2018490, NT2NE2018594, NT2NE2018739, NT2NE2018916, NT2NE2019056, NT2NE2019076, NT2NE2019091, NT2NE2019099, NT2RI1000016, NT2RI1000035, NT2RI1000048, NT2RI1000127, NT2RI1000164, NT2RI1000172, NT2RI2000004, NT2RI2000007, NT2RI2000116, NT2RI2000117, NT2RI2000128, NT2RI2000133, NT2RI2000167, NT2RI2000173, NT2RI2000187, NT2RI2000270, NT2RI2000276, NT2RI2000294, NT2RI2000313, NT2RI2000341, NT2RI2000344, NT2RI2000348, NT2RI2000421, NT2RI2000480, NT2RI2000575, NT2RI2000578, NT2RI2000588, NT2RI2000597, NT2RI2000669, NT2RI2000688, NT2RI2000704, NT2RI2000738, NT2RI2000775, NT2RI2000822,

NT2RI2000829, NT2RI2000841, NT2RI200.0865, NT2RI2000932, NT2RI2000974, NT2RI2000987, NT2RI2001010, NT2RI2001017, NT2RI2001030, NT2RI2001083, NT2RI2001167, NT2RI2001178, NT2RI2001235, NT2RI2001244, NT2RI2001307, NT2RI2001342, NT2RI2001410, NT2RI2001425, NT2RI2001449, NT2RI2001450, NT2RI2001474, NT2RI2001519, NT2RI2001595, NT2RI2001624, NT2RI2001731, NT2RI2001769, NT2RI2001782, NT2RI2001836, NT2RI2001846, NT2RI2001866, NT2RI2001910, NT2RI2001952, NT2RI2002022, NT2RI2002041, NT2RI2002120, NT2RI2002153, NT2RI2002179, NT2RI2002243, NT2RI2002252, NT2RI2002260, NT2RI2002266, NT2RI2002270, NT2RI2002334, NT2RI2002359, NT2RI2002447, NT2RI2002481, NT2RI2002507, NT2RI2002517, NT2RI2002530, NT2RI2002540,

NT2RI2002564, NT2RI2002585, NT2RI2002592, NT2RI2002602, NT2RI2002654, NT2RI2002699, NT2RI2002729, NT2RI2002802, NT2RI2002819, NT2RI2002822, NT2RI2002852, NT2RI2002865, NT2RI2002923, NT2RI2002926, NT2RI2002970, NT2RI2003011, NT2RI2003019, NT2RI2003051, NT2RI2003067, NT2RI2003184, NT2RI2003222, NT2RI2003301, NT2RI2003317, NT2RI2003344, NT2RI2003360, NT2RI2003361, NT2RI2003383, NT2RI2003392, NT2RI2003399, NT2RI2003419, NT2RI2003481, NT2RI2003487, NT2RI2003556, NT2RI2003655, NT2RI2003667, NT2RI2003678, NT2RI2003751, NT2RI2003884, NT2RI2003973, NT2RI2003993, NT2RI2004078, NT2RI2004079, NT2RI2004093, NT2RI2004136, NT2RI2004157, NT2RI2004190, NT2RI2004209, NT2RI2004230, NT2RI2004284, NT2RI2004290,

NT2RI2004304, NT2RI2004312, NT2RI2004381, NT2RI2004398, NT2RI2004410, NT2RI2004442, NT2RI2004468, NT2RI2004535, NT2RI2004606, NT2RI2004618, NT2RI2004783, NT2RI2004818, NT2RI2004840, NT2RI2004884, NT2RI2004916, NT2RI2004962, NT2RI2004984, NT2RI2004985, NT2RI2005048, NT2RI2005061, NT2RI2005069, NT2RI2005087, NT2RI2005096, NT2RI2005116, NT2RI2005130, NT2RI2005166, NT2RI2005246, NT2RI2005315, NT2RI2005335, NT2RI2005353, NT2RI2005358, NT2RI2005368, NT2RI2005382, NT2RI2005473, NT2RI2005520, NT2RI2005564, NT2RI2005579, NT2RI2005621, NT2RI2005628, NT2RI2005647, NT2RI2005670, NT2RI200.5710, NT2RI2005772, NT2RI2005811, NT2RI2005814, NT2RI2005816, NT2RI2005818, NT2RI2005851, NT2RI2005966, NT2RI2006071,

NT2RI2006081, NT2RI2006126, NT2RI2006127, NT2RI2006183, NT2RI2006257, NT2RI2006271, NT2RI2006294, NT2RI2006345, NT2RI2006403, NT2RI2006445, NT2RI2006487, NT2RI2006506, NT2RI2006553, NT2RI2006565, NT2RI2006640, NT2RI2006647, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006838, NT2RI2006943, NT2RI2006973, NT2RI2007046, NT2RI2007048, NT2RI2007116, NT2RI2007148, NT2RI2007214, NT2RI2007277, NT2RI2007303, NT2RI2007334, NT2RI2007377, NT2RI2007386, NT2RI2007406, NT2RI2007439, NT2RI2007445, NT2RI2007468, NT2RI2007469, NT2RI2007507, NT2RI2007572, NT2RI2007593, NT2RI2007729, NT2RI2007751,

NT2RI2007754, NT2RI2007853, NT2RI2007877, NT2RI2007879, NT2RI2007891, NT2RI2007956, NT2RI2007965, NT2RI2008007, NT2RI2008050, NT2RI2008141, NT2RI2008188, NT2RI2008233, NT2RI2008396, NT2RI2008455, NT2RI2008502, NT2RI2008566, NT2RI2008598, NT2RI2008656, NT2RI2008714, NT2RI2008724, NT2RI2008801, NT2RI2008808, NT2RI2008841, NT2RI2008942, NT2RI2008952, NT2RI2009005, NT2RI2009066, NT2RI2009101, NT2RI2009103, NT2RI2009144, NT2RI2009151, NT2RI2009194, NT2RI2009215, NT2RI2009233, NT2RI2009259, NT2RI2009289, NT2RI2009301, NT2RI2009402, NT2RI2009406, NT2RI2009517, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803,

NT2RI2012350, NT2RI2012659, NT2RI2012990, NT2RI2013345, NT2RI2013357, NT2RI2013373, NT2RI2014247, NT2RI2014551, NT2RI2014683, NT2RI2014733, NT2RI2015239, NT2RI2015342, NT2RI2016128, NT2RI2016134, NT2RI2018311, NT2RI2018363, NT2RI2018883, NT2RI2018950, NT2RI2019751, NT2RI2019826, NT2RI2020390, NT2RI2020703, NT2RI2021463, NT2RI2021625, NT2RI2022584, NT2RI2023303, NT2RI2023671, NT2RI2024008, NT2RI2024313, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975, NT2RI2028213, NT2RI2028222, NT2RI2028537,

NT2RI2028642, NT2RI3000174, NT2RI3000213, NT2RI3000423, NT2RI3000622, NT2RI3001132, NT2RI3001263, NT2RI3001279, NT2RI3001348, NT2RI3001445, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005724, NT2RI3005861, NT2RI3005923, NT2RI3005928, NT2RI3006132, NT2RI3006171, NT2RI3006284, NT2RI3006340, NT2RI3006376, NT2RI3006666, NT2RI3006673, NT2RI3006796, NT2RI3007065, NT2RI3007095, NT2RI3007158, NT2RI3007167, NT2RI3007213,

NT2RI3007291, NT2RI3007443, NT2RI3007543, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000005, NT2RP6000008, NT2RP6000017, NT2RP6000032, NT2RP6000033, NT2RP6000039, NT2RP6000059, NT2RP6000060, NT2RP6000077, NT2RP6000085, NT2RP6000086, NT2RP6000100, NT2RP6000109, NT2RP6000110, NT2RP6000119, NT2RP6000127, NT2RP6000130, NT2RP6000140, NT2RP6000150, NT2RP7000041, NT2RP7000069, NT2RP7000086, NT2RP7000109, NT2RP7000173, NT2RP7000238, NT2RP7000259, NT2RP7000311, NT2RP7000359, NT2RP7000364, NT2RP7000380, NT2RP7000391, NT2RP7000425,

NT2RP7000477, NT2RP7000579, NT2RP7000586, NT2RP7000600, NT2RP7000624, NT2RP7000812, NT2RP7000926, NT2RP7001074, NT2RP7001080, NT2RP7001156, NT2RP7001231, NT2RP7001319, NT2RP7001335, NT2RP7001364, NT2RP7001475, NT2RP7001532, NT2RP7001591, NT2RP7001602, NT2RP7001678, NT2RP7001856, NT2RP7001962, NT2RP7002028, NT2RP7002064, NT2RP7002151, NT2RP7002163, NT2RP7002243, NT2RP7002376, NT2RP7002450, NT2RP7002478, NT2RP7002554, NT2RP7002594, NT2RP7002603, NT2RP7002650, NT2RP7002738, NT2RP7002779, NT2RP7002802, NT2RP7002841, NT2RP7002875, NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003203,

NT2RP7003319, NT2RP7003439, NT2RP7003511, NT2RP7003629, NT2RP7003632, NT2RP7003647, NT2RP7003680, NT2RP7003724, NT2RP7003960, NT2RP7004027, NT2RP7004080, NT2RP7004114, NT2RP7004123, NT2RP7004196, NT2RP7004204, NT2RP7004233, NT2RP7004260, NT2RP7004352, NT2RP7004358, NT2RP7004396, NT2RP7004428, NT2RP7004481, NT2RP7004559, NT2RP7004641, NT2RP7004656, NT2RP7004687, NT2RP7004722, NT2RP7004728, NT2RP7004766, NT2RP7004790, NT2RP7004911, NT2RP7004915, NT2RP7004961, NT2RP7004964, NT2RP7004975, NT2RP7005118, NT2RP7005205, NT2RP7005219, NT2RP7005323, NT2RP7005468, NT2RP7005493, NT2RP7005529, NT2RP7005629, NT2RP7005631, NT2RP7005669, NT2RP7005675, NT2RP7005750, NT2RP7005846, NT2RP7006033, NT2RP7006160,

NT2RP7006162, NT2RP7006188, NT2RP7006223, NT2RP7006263, NT2RP7006296, NT2RP7006304, NT2RP7006374, NT2RP7006395, NT2RP7006408, NT2RP7006457, NT2RP7006490, NT2RP7006547, NT2RP7006601, NT2RP7006619, NT2RP7006621, NT2RP7006636, NT2RP7006701, NT2RP7006853, NT2RP7006886, NT2RP7006887, NT2RP7006986, NT2RP7006995, NT2RP7007114, NT2RP7007177, NT2RP7007221, NT2RP7007226, NT2RP7007252, NT2RP7007269, NT2RP7007310, NT2RP7007359, NT2RP7007381, NT2RP7007406, NT2RP7007480, NT2RP7007504, NT2RP7007537, NT2RP7007580, NT2RP7007594, NT2RP7007610, NT2RP7007617, NT2RP7007643, NT2RP7007766, NT2RP7007925, NT2RP7007930, NT2RP7008013, NT2RP7008015, NT2RP7008133, NT2RP7008137, NT2RP7008142, NT2RP7008144, NT2RP7008161,

NT2RP7008190, NT2RP7008315, NT2RP7008360, NT2RP7008396, NT2RP7008406, NT2RP7008435, NT2RP7008441, NT2RP7008454, NT2RP7008487, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009087, NT2RP7009108, NT2RP7009147, NT2RP7009149, NT2RP7009168, NT2RP7009215, NT2RP7009225, NT2RP7009236, NT2RP7009253, NT2RP7009267, NT2RP7009322, NT2RP7009363, NT2RP7009370, NT2RP7009373, NT2RP7009397, NT2RP7009429, NT2RP7009439, NT2RP7009466, NT2RP7009481, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275,

NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7013002, NT2RP7013374, NT2RP7013499, NT2RP7013573, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971,

NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000087, NTONG1000123, NTONG1000130, NTONG1000161,

NTONG1000172, NTONG1000213, NTONG1000214, NTONG1000247, NTONG1000264, NTONG2000040, NTONG2000107, NTONG2000231, NTONG2000334, NTONG2000363, NTONG2000413, NTONG2000492, NTONG2000499, NTONG2000531, NTONG2000583, NTONG2000800, NTONG2000876, NTONG2000878, NTONG2000966, NTONG2000985, NTONG2001014, NTONG2001079, NTONG2001137, NTONG2001157, NTONG2001173, NTONG2001220, NTONG2001222, NTONG2001362, NTONG2001428, NTONG2001532, NTONG2001550, NTONG2001587, NTONG2001619, NTONG2001642, NTONG2001657, NTONG2001676, NTONG2001762, NTONG2002278, NTONG2002807, NTONG2002948, NTONG2002970, NTONG2002985, NTONG2003158, NTONG2003210, NTONG2003316, NTONG2003409, NTONG2003805, NTONG2003839, NTONG2003852, NTONG2003897,

NTONG2004095, NTONG2004308, NTONG2004521, NTONG2004614, NTONG2004690, NTONG2004829, NTONG2004918, NTONG2004991, NTONG2005062, NTONG2005086, NTONG2005137, NTONG2005265, NTONG2005277, NTONG2005363, NTONG2005373, NTONG2005391, NTONG2005480, NTONG2005497, NTONG2005577, NTONG2005657, NTONG2005801, NTONG2005897, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008672, NTONG2008862, NTONG2008939,

NTONG2008944, NTONG2009060, NTONG2009068, NTONG2009229, NTONG2009233, NTONG2009576, OCBBF1000016, OCBBF1000030, OCBBF1000042, OCBBF1000049, OCBBF1000054, OCBBF1000067, OCBBF1000080, OCBBF1000085, OCBBF1000091, OCBBF1000104, OCBBF1000118, OCBBF1000119, OCBBF1000122, OCBBF1000130, OCBBF1000138, OCBBF1000145, OCBBF1000153, OCBBF1000175, OCBBF1000185, OCBBF1000188, OCBBF1000192, OCBBF1000254, OCBBF1000315, OCBBF2000015, OCBBF2000126, OCBBF2000178, OCBBF2000231, OCBBF2000277, OCBBF2000323, OCBBF2000452, OCBBF2000677, OCBBF2000703, OCBBF2000719, OCBBF2000824, OCBBF2000831, OCBBF2000846, OCBBF2000904, OCBBF2000986, OCBBF2000998, OCBBF2001075, OCBBF2001101, OCBBF2001124, OCBBF2001140, OCBBF2001166,

OCBBF2001176, OCBBF2001186, OCBBF2001210, OCBBF2001252, OCBBF2001307, OCBBF2001323, OCBBF2001389, OCBBF2001402, OCBBF2001408, OCBBF2001416, OCBBF2001473, OCBBF2001492, OCBBF2001494, OCBBF2001527, OCBBF2001528, OCBBF2001586, OCBBF2001639, OCBBF2001681, OCBBF2001687, OCBBF2001706, OCBBF2001749, OCBBF2001910, OCBBF2001931, OCBBF2001961, OCBBF2001983, OCBBF2002015, OCBBF2002083, OCBBF2002124, OCBBF2002161, OCBBF2002290, OCBBF2002357, OCBBF2002615, OCBBF2002626, OCBBF2002656, OCBBF2002663, OCBBF2002805, OCBBF2002865, OCBBF2002920, OCBBF2002980, OCBBF2003028, OCBBF2003052, OCBBF2003091, OCBBF2003246, OCBBF2003327, OCBBF2003518, OCBBF2003543, OCBBF2003593, OCBBF2003744, OCBBF2003819, OCBBF2003925,

OCBBF2004038, OCBBF2004147, OCBBF2004168, OCBBF2004259, OCBBF2004273, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843, OCBBF2005901, OCBBF2006005, OCBBF2006030, OCBBF2006058, OCBBF2006113, OCBBF2006148, OCBBF2006151, OCBBF2006154, OCBBF2006172, OCBBF2006214, OCBBF2006241, OCBBF2006313, OCBBF2006388, OCBBF2006438, OCBBF2006567, OCBBF2006624, OCBBF2006639,

OCBBF2006764, OCBBF2006972, OCBBF2006987, OCBBF2007028, OCBBF2007039, OCBBF2007051, OCBBF2007068, OCBBF2007114, OCBBF2007121, OCBBF2007184, OCBBF2007194, OCBBF2007196, OCBBF2007224, OCBBF2007238, OCBBF2007414, OCBBF2007428, OCBBF2007478, OCBBF2007485, OCBBF2007520, OCBBF2007555, OCBBF2007610, OCBBF2007756, OCBBF2007762, OCBBF2007829, OCBBF2007892, OCBBF2007931, OCBBF2007946, OCBBF2008041, OCBBF2008116, OCBBF2008138, OCBBF2008144, OCBBF2008283, OCBBF2008330, OCBBF2008340, OCBBF2008366, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770, OCBBF2008775, OCBBF2008790, OCBBF2008814,

OCBBF2008822, OCBBF2009095, OCBBF2009115, OCBBF2009242, OCBBF2009301, OCBBF2009352, OCBBF2009391, OCBBF2009424, OCBBF2009536, OCBBF2009571, OCBBF2009583, OCBBF2009603, OCBBF2009772, OCBBF2009788, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010819, OCBBF2010843, OCBBF2010858, OCBBF2010863, OCBBF2010871, OCBBF2010931, OCBBF2010945, OCBBF2010978, OCBBF2010989, OCBBF2011021, OCBBF2011073, OCBBF2011137, OCBBF2011160, OCBBF2011177, OCBBF2011228, OCBBF2011232, OCBBF2011283, OCBBF2011311, OCBBF2011625, OCBBF2011669, OCBBF2011685, OCBBF2011706, OCBBF2011722,

OCBBF2011759, OCBBF2011767, OCBBF2011855, OCBBF2011872, OCBBF2011887, OCBBF2011897, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320, OCBBF2012436, OCBBF2012525, OCBBF2012678, OCBBF2012704, OCBBF2012714, OCBBF2012755, OCBBF2012812, OCBBF2012821, OCBBF2012881, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013481, OCBBF2013507, OCBBF2013721, OCBBF2013789, OCBBF2013843, OCBBF2013883, OCBBF2013926, OCBBF2014052, OCBBF2014064, OCBBF2014089, OCBBF2014322, OCBBF2014386, OCBBF2014576,

OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503, OCBBF2015506, OCBBF2015599, OCBBF2015645, OCBBF2015659, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2017035, OCBBF2017055, OCBBF2017069, OCBBF2017306, OCBBF2017325, OCBBF2017458, OCBBF2017489, OCBBF2017516, OCBBF2017665, OCBBF2017754, OCBBF2017815, OCBBF2017882, OCBBF2017888, OCBBF2017893, OCBBF2017920, OCBBF2018012, OCBBF2018014, OCBBF2018167, OCBBF2018206,

OCBBF2018229, OCBBF2018234, OCBBF2018362, OCBBF2018380, OCBBF2018563, OCBBF2018581, OCBBF2018618, OCBBF2018663, OCBBF2018687, OCBBF2018827, OCBBF2018828, OCBBF2018873, OCBBF2018934, OCBBF2018956, OCBBF2019108, OCBBF2019195, OCBBF2019327, OCBBF2019684, OCBBF2019761, OCBBF2019823, OCBBF2019950, OCBBF2020048, OCBBF2020318, OCBBF2020343, OCBBF2020453, OCBBF2020741, OCBBF2020801, OCBBF2020825, OCBBF2020838, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2024850, OCBBF2025028, OCBBF2025115, OCBBF2025198,

OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413,

OCBBF2033869, OCBBF2033948, OCBBF2034333, OCBBF2034529, OCBBF2034637, OCBBF2034906, OCBBF2035110, OCBBF2035214, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314,

OCBBF3004487, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091, PANCR1000185, PEBLM1000024, PEBLM1000029, PEBLM1000062, PEBLM1000066, PEBLM1000068, PEBLM1000071, PEBLM1000083, PEBLM1000144, PEBLM1000147, PEBLM1000174, PEBLM1000180, PEBLM2000030, PEBLM2000112, PEBLM2000147, PEBLM2000170, PEBLM2000180, PEBLM2000213, PEBLM2000248, PEBLM2000267, PEBLM2000270, PEBLM2000308, PEBLM2000321, PEBLM2000326, PEBLM2000333, PEBLM2000338, PEBLM2000502, PEBLM2001312, PEBLM2001465, PEBLM2001488,

PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004452, PEBLM2004497, PEBLM2004733, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006366, PEBLM2006709, PEBLM2006912, PEBLM2007112, PEBLM2007140, PEBLM2007188, PEBLM2007437, PEBLM2007447, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387,

PERIC2000422, PERIC2000473, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086,

PERIC2009329, PERIC2009566, PLACE5000001, PLACE5000037, PLACE5000058, PLACE5000067, PLACE5000068, PLACE5000070, PLACE5000080, PLACE5000105, PLACE5000115, PLACE5000116, PLACE5000129, PLACE5000139, PLACE5000153, PLACE5000159, PLACE5000171, PLACE5000172, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000145, PLACE6000191, PLACE6000205, PLACE6000231, PLACE6000263, PLACE6000296, PLACE6000314, PLACE6000325, PLACE6000348, PLACE6000424, PLACE6000426, PLACE6000429, PLACE6000450, PLACE6000523, PLACE6000550, PLACE6000552, PLACE6000555, PLACE6000619,

PLACE6000953, PLACE6001118, PLACE6001153, PLACE6001185, PLACE6001262, PLACE6001281, PLACE6001294, PLACE6001536, PLACE6001557, PLACE6001712, PLACE6001740, PLACE6001823, PLACE6001923, PLACE6001925, PLACE6001933, PLACE6002016, PLACE6002102, PLACE6002157, PLACE6002312, PLACE6002323, PLACE6002345, PLACE6002462, PLACE6002647, PLACE6002692, PLACE6002699, PLACE6002710, PLACE6002871, PLACE6002949, PLACE6003004, PLACE6003038, PLACE6003077, PLACE6003094, PLACE6003109, PLACE6003204, PLACE6003218, PLACE6003372, PLACE6003393, PLACE6003399, PLACE6003539, PLACE6003621, PLACE6003705, PLACE6003740, PLACE6003745, PLACE6003771, PLACE6003796, PLACE6003839, PLACE6004101, PLACE6004219, PLACE6004336, PLACE6004368,

PLACE6004380, PLACE6004396, PLACE6004397, PLACE6004454, PLACE6004491, PLACE6004663, PLACE6004687, PLACE6004738, PLACE6004931, PLACE6004966, PLACE6004993, PLACE6005007, PLACE6005029, PLACE6005108, PLACE6005231, PLACE6005234, PLACE6005283, PLACE6005294, PLACE6005328, PLACE6005351, PLACE6005423, PLACE6005482, PLACE6005487, PLACE6005535, PLACE6005546, PLACE6005559, PLACE6005579, PLACE6005691, PLACE6005786, PLACE6006077, PLACE6006137, PLACE6006158, PLACE6006186, PLACE6006217, PLACE6006222, PLACE6006266, PLACE6006287, PLACE6006394, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309,

PLACE6007373, PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008824, PLACE6008989, PLACE6009006, PLACE6009008, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011057, PLACE6011102, PLACE6011156,

PLACE6011211, PLACE6011260, PLACE6011334, PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012574, PLACE6012908, PLACE6012936, PLACE6012942, PLACE6013222, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016383, PLACE6016984, PLACE6017002, PLACE6017431,

PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6018938, PLACE6019195, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004103, PLACE7004961, PLACE7005169,

PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000110, PROST1000123, PROST1000136, PROST1000145, PROST1000152, PROST1000167, PROST1000184, PROST1000217, PROST1000220, PROST1000262, PROST1000272, PROST1000298, PROST1000322, PROST1000334, PROST1000343, PROST1000362, PROST1000377, PROST1000383, PROST1000412, PROST1000419, PROST1000451, PROST1000485, PROST1000493, PROST1000512, PROST1000527, PROST1000528, PROST1000536,

PROST1000559, PROST1000575, PROST2000036, PROST2000053, PROST2000067, PROST2000079, PROST2000105, PROST2000143, PROST2000162, PROST2000165, PROST2000176, PROST2000203, PROST2000206, PROST2000212, PROST2000241, PROST2000267, PROST2000273, PROST2000274, PROST2000277, PROST2000284, PROST2000325, PROST2000337, PROST2000432, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002, PROST2001116, PROST2001138, PROST2001213, PROST2001414, PROST2001415, PROST2001465, PROST2001521, PROST2001540, PROST2001599, PROST2001611, PROST2001636, PROST2001796, PROST2001805, PROST2001823,

PROST2001899, PROST2.001997, PROST2001998, PROST2002101, PROST2002162, PROST2002212, PROST2002227, PROST2002338, PROST2002488, PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002927, PROST2002960, PROST2003102, PROST2003117, PROST2003210, PROST2003302, PROST2003303, PROST2003324, PROST2003340, PROST2003396, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142,

PROST2004146, PROST2004161, PROST2004251, PROST2004332, PROST2004481, PROST2004526, PROST2004570, PROST2004727, PROST2004739, PROST2004742, PROST2004744, PROST2004817, PROST2005039, PROST2005067, PROST2005076, PROST2005093, PROST2005121, PROST2005131, PROST2005228, PROST2005285, PROST2005426, PROST2005466, PROST2005604, PROST2005630, PROST2005683, PROST2005788, PROST2005793, PROST2005880, PROST2005886, PROST2005904, PROST2005919, PROST2005943, PROST2006008, PROST2006020, PROST2006030, PROST2006060, PROST2006196, PROST2006201, PROST2006260, PROST2006343, PROST2006450, PROST2006491, PROST2006510, PROST2006536, PROST2006579, PROST2006688, PROST2006737, PROST2006766, PROST2006782, PROST2006988,

PROST2007122, PROST2007200, PROST2007237, PROST2007248, PROST2007289, PROST2007317, PROST2007328, PROST2007382, PROST2007389, PROST2007416, PROST2007528, PROST2007612, PROST2007818, PROST2007871, PROST2007950, PROST2007956, PROST2007974, PROST2007988, PROST2008079, PROST2008088, PROST2008243, PROST2008245, PROST2008268, PROST2008271, PROST2008447, PROST2008468, PROST2008472, PROST2008489, PROST2008516, PROST2008724, PROST2008770, PROST2008835, PROST2008993, PROST2009022, PROST2009208, PROST2009222, PROST2009254, PROST2009273, PROST2009347, PROST2009365, PROST2009388, PROST2009400, PROST2009470, PROST2009483, PROST2009571, PROST2009731, PROST2009736, PROST2009795, PROST2009901, PROST2009909,

PROST2010046, PROST2010219, PROST2010250, PROST2010318, PROST2010326, PROST2010382, PROST2010400, PROST2010579, PROST2010606, PROST2010782, PROST2010885, PROST2011012, PROST2011038, PROST2011105, PROST2011297, PROST2011410, PROST2011439, PROST2011482, PROST2011577, PROST2011631, PROST2011660, PROST2012005, PROST2012007, PROST2012016, PROST2012088, PROST2012140, PROST2012157, PROST2012190, PROST2012249, PROST2012353, PROST2012400, PROST2012448, PROST2012504, PROST2012542, PROST2012550, PROST2012740, PROST2012888, PROST2012890, PROST2013032, PROST2013121, PROST2013260, PROST2013327, PROST2013531, PROST2013605, PROST2013627, PROST2013873, PROST2013880, PROST2014422, PROST2014585, PROST2014601,

PROST2014659, PROST2014916, PROST2014925, PROST2015124, PROST2015137, PROST2015198, PROST2015243, PROST2015246, PROST2015251, PROST2015287, PROST2015332, PROST2015457, PROST2015537, PROST2015545, PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016918, PROST2017098, PROST2017128, PROST2017203, PROST2017367, PROST2017413, PROST2017441, PROST2017529, PROST2017578, PROST2017612, PROST2017617, PROST2017700, PROST2017701,

PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019296, PROST2019398, PROST2019487, PROST2019781, PUAEN1000039, PUAEN1000057, PUAEN1000081, PUAEN1000085, PUAEN1000087, PUAEN1000121, PUAEN1000147, PUAEN1000161, PUAEN1000175, PUAEN1000239, PUAEN1000275, PUAEN1000276, PUAEN1000288, PUAEN2000080, PUAEN2000152, PUAEN2000175, PUAEN2000247, PUAEN2000312, PUAEN2000374, PUAEN2000394, PUAEN2000497, PUAEN2000594, PUAEN2000684, PUAEN2000942, PUAEN2001260, PUAEN2001345, PUAEN2001526, PUAEN2001586, PUAEN2001882, PUAEN2002473,

PUAEN2002489, PUAEN2002552, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005588, PUAEN2005930, PUAEN2006029, PUAEN2006328, PUAEN2006335, PUAEN2006578, PUAEN2006639, PUAEN2006761, PUAEN2007044, PUAEN2007350, PUAEN2007785, PUAEN2007898, PUAEN2008123, PUAEN2008228, PUAEN2008314, PUAEN2008515, PUAEN2008815, PUAEN2008939, PUAEN2008980, PUAEN2008985, PUAEN2009174, PUAEN2009348, PUAEN2009534, PUAEN2009655, PUAEN2009795, PUAEN2009852, PUAEN2010346, PUAEN2010824, RECTM1000051, RECTM1000141, RECTM2000220, RECTM2000349,

RECTM2000433, RECTM2001307, RECTM2001347, RECTM2001519, RECTM2001666, RECTM2001691, RECTM2001749, RECTM2002172, RECTM2002602, RECTM2002632, SYNOV1000049, SYNOV1000118, SYNOV1000128, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001144, SYNOV2001212, SYNOV2001251, SYNOV2001300, SYNOV2001356, SYNOV2001374, SYNOV2001390, SYNOV2001451, SYNOV2001457, SYNOV2001468, SYNOV2001581, SYNOV2001660, SYNOV2003326,

SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001153, SYNOV4001224, SYNOV4001326, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981, SYNOV4004184, SYNOV4004741, SYNOV4004823,

SYNOV4004914, SYNOV4005256, SYNOV4005570, SYNOV4005739, SYNOV4005889, SYNOV4005981, SYNOV4005989, SYNOV4006256, SYNOV4006542, SYNOV4007012, SYNOV4007215, SYNOV4007360, SYNOV4007430, SYNOV4007521, SYNOV4007553, SYNOV4007711, SYNOV4008245, SYNOV4008336, SYNOV4009129, SYNOV4009139, SYNOV4009298, T1ESE2000116, T1ESE2000609, T1ESE2000904, T1ESE2002665, TBAES2000004, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543,

TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000431, TCERX2000613, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2001953, TESOP2002005, TESOP2002110, TESOP2002150,

TESOP2002273, TESOP2002451, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006704, TESOP2006746, TESOP2006865, TESOP2006893, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018,

TESTI1000019, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000168, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018, TESTI2000044, TESTI2000100, TESTI2000117, TESTI2000120, TESTI2000147, TESTI2000154, TESTI2000172, TESTI2000177, TESTI2000179,

TESTI2000207, TESTI2000208, TESTI2000221, TESTI2000238, TESTI2000253, TESTI2000272, TESTI2000278, TESTI2000349, TESTI2000356, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000784, TESTI2000808, TESTI2000819, TESTI2000830, TESTI2000840, TESTI2000849, TESTI2000850, TESTI2000874, TESTI2000883, TESTI2000909, TESTI2000951, TESTI2000970, TESTI2000981, TESTI2001008, TESTI2001049, TESTI2001082,

TESTI2001120, TESTI2001134, TESTI2001141, TESTI2001180, TESTI2001208, TESTI2001229, TESTI2001236, TESTI2001237, TESTI2001269, TESTI2001306, TESTI2001313, TESTI2001330, TESTI2001345, TESTI2001352, TESTI2001364, TESTI2001412, TESTI2001420, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001869, TESTI2001879, TESTI2001894, TESTI2001898, TESTI2001915, TESTI2001950, TESTI2001959, TESTI2001968, TESTI2001985,

TESTI2001991, TESTI2002002, TESTI2002012, TESTI2002036, TESTI2002048, TESTI2002057, TESTI2002081, TESTI2002090, TESTI2002105, TESTI2002149, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002365, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002698, TESTI2002700, TESTI2002707, TESTI2002724, TESTI2002729, TESTI2002752, TESTI2002753, TESTI2002789, TESTI2002802, TESTI2002806,

TESTI2002840, TESTI2002866, TESTI2002877, TESTI2002910, TESTI2002912, TESTI2002928, TESTI2002937, TESTI2002965, TESTI2002993, TESTI2003005, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003413, TESTI2003418,

TESTI2003419, TESTI2003432, TESTI2003475, TESTI2003476, TESTI2003498, TESTI2003512, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004031, TESTI2004073, TESTI2004080, TESTI2004085, TESTI2004089, TESTI2004111, TESTI2004119, TESTI2004122, TESTI2004153, TESTI2004163, TESTI2004169, TESTI2004207, TESTI2004215, TESTI2004227, TESTI2004229,

TESTI2004243, TESTI2004255, TESTI2004287, TESTI2004295, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004783, TESTI2004791, TESTI2004793, TESTI2004860, TESTI2004906, TESTI2004920, TESTI2004936, TESTI2004941, TESTI2004971, TESTI2004982, TESTI2004993, TESTI2004994, TESTI2004999, TESTI2005025, TESTI2005040, TESTI2005060, TESTI2005072, TESTI2005085, TESTI2005112, TESTI2005120,

TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005731, TESTI2005739, TESTI2005742, TESTI2005767, TESTI2005775, TESTI2005784, TESTI2005788, TESTI2005827, TESTI2005835, TESTI2005860, TESTI2005892, TESTI2005908, TESTI2005911, TESTI2005933, TESTI2005937, TESTI2005948, TESTI2005979,

TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006111, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006360, TESTI2006409, TESTI2006425, TESTI2006437, TESTI2006453, TESTI2006457, TESTI2006460, TESTI2006465, TESTI2006483, TESTI2006499, TESTI2006523, TESTI2006543, TESTI2006565, TESTI2006572, TESTI2006617, TESTI2006624, TESTI2006628, TESTI2006633, TESTI2006643, TESTI2006648,

TESTI2006659, TESTI2006660, TESTI2006665, TESTI2006667, TESTI2006677, TESTI2006720, TESTI2006735, TESTI2006744, TESTI2006748, TESTI2006774, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007211, TESTI2007220, TESTI2007234, TESTI2007311, TESTI2007320, TESTI2007346, TESTI2007358, TESTI2007402, TESTI2007405, TESTI2007452, TESTI2007464, TESTI2007466, TESTI2007480, TESTI2007490, TESTI2007502, TESTI2007524, TESTI2007533, TESTI2007576, TESTI2007613, TESTI2007657, TESTI2007685, TESTI2007692,

TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621, TESTI2008636, TESTI2008657, TESTI2008684, TESTI2008715, TESTI2008762, TESTI2008774, TESTI2008786,

TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009474, TESTI2009477, TESTI2009497, TESTI2009502, TESTI2009511, TESTI2009520, TESTI2009544, TESTI2009545, TESTI2009551, TESTI2009577, TESTI2009585, TESTI2009588, TESTI2009683, TESTI2009727, TESTI2009739, TESTI2009812, TESTI2009835, TESTI2009853, TESTI2009935, TESTI2009977, TESTI2009987, TESTI2010009, TESTI2010154, TESTI2010180,

TESTI2010239, TESTI2010280, TESTI2010354, TESTI2010396, TESTI2010400, TESTI2010409, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011612, TESTI2011665, TESTI2011680, TESTI2011683, TESTI2011750, TESTI2011840, TESTI2011846, TESTI2011915, TESTI2011928, TESTI2011971, TESTI2012019, TESTI2012050, TESTI2012090, TESTI2012095, TESTI2012152, TESTI2012155,

TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257, TESTI2013264, TESTI2013268, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610,

TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014577, TESTI2014578, TESTI2014716, TESTI2014780, TESTI2014800, TESTI2014838, TESTI2014843, TESTI2014979, TESTI2014988, TESTI2015042, TESTI2015105, TESTI2015180, TESTI2015213, TESTI2015246, TESTI2015249, TESTI2015327, TESTI2015331, TESTI2015335, TESTI2015350, TESTI2015375, TESTI2015437, TESTI2015590, TESTI2015610, TESTI2015647, TESTI2015675, TESTI2015710, TESTI2015752,

TESTI2015830, TESTI2015914, TESTI2015938, TESTI2015947, TESTI2015962, TESTI2015987, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582,

TESTI2017584, TESTI2017645, TESTI2017727, TESTI2017740, TESTI2017775, TESTI2017816, TESTI2017832, TESTI2017923, TESTI2017932, TESTI2017951, TESTI2018014, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018867, TESTI2018941, TESTI2018957, TESTI2018974, TESTI2018975, TESTI2019042, TESTI2019077, TESTI2019228, TESTI2019257, TESTI2019280, TESTI2019282, TESTI2019308, TESTI2019336, TESTI2019352, TESTI2019406, TESTI2019430, TESTI2019455,

TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020871, TESTI2020905, TESTI2020906, TESTI2020946, TESTI2020956, TESTI2020981, TESTI2020999, TESTI2021003, TESTI2021038, TESTI2021057, TESTI2021077, TESTI2021112, TESTI2021114, TESTI2021116, TESTI2021118,

TESTI2021122, TESTI2021124, TESTI2021138, TESTI2021297, TESTI2021315, TESTI2021358, TESTI2021425, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194,

TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024936, TESTI2024978, TESTI2024999, TESTI2025006, TESTI2025013, TESTI2025022, TESTI2025063, TESTI2025117, TESTI2025144, TESTI2025161,

TESTI2025174, TESTI2025229, TESTI2025269, TESTI2025365, TESTI2025403, TESTI2025409, TESTI2025422, TESTI2025448, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605, TESTI2026647, TESTI2026674, TESTI2026794,

TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028659, TESTI2028662, TESTI2028670,

TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029259, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067,

TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262,

TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036913, TESTI2036922, TESTI2036951, TESTI2036965, TESTI2036969, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048,

TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039177, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330,

TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276, TESTI2044306, TESTI2044309, TESTI2044413, TESTI2044418, TESTI2044754, TESTI2044788, TESTI2044796,

TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047792, TESTI2047801, TESTI2047818, TESTI2047824, TESTI2047885, TESTI2047930, TESTI2048105, TESTI2048109, TESTI2048465,

TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242, TESTI2053399, TESTI2053526, TESTI2053561, TESTI2053621, TESTI2053667, TESTI2053723,

TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001348, TESTI4001441, TESTI4001467, TESTI4001473, TESTI4001517, TESTI4001527, TESTI4001561, TESTI4001569, TESTI4001604, TESTI4001665, TESTI4001679,

TESTI4001795, TESTI4001923, TESTI4001925, TESTI4001984, TESTI4002003, TESTI4002072, TESTI4002141, TESTI4002195, TESTI4002290, TESTI4002319, TESTI4002491, TESTI4002520, TESTI4002552, TESTI4002647, TESTI4002703, TESTI4002754, TESTI4002799, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003703, TESTI4003733, TESTI4003796, TESTI4003944, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158, TESTI4005317,

TESTI4005322, TESTI4005399, TESTI4005470, TESTI4005500, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007965, TESTI4008007,

TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789,

TESTI4010817, TESTI4010831, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013602, TESTI4013667, TESTI4013675, TESTI4013685, TESTI4013735, TESTI4013742, TESTI4013774, TESTI4013817, TESTI4013830, TESTI4013852, TESTI4013894,

TESTI4013924, TESTI4013960, TESTI4013962, TESTI4014159, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229,

TESTI4017254, TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456,

TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024245, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700,

TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029671, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128,

TESTI4032270, TESTI4032375, TESTI4032834, TESTI4032856, TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258,

TESTI4038284, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043223,

TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746,

TESTI4047808, THYMU1000002, THYMU1000016, THYMU1000017, THYMU1000025, THYMU1000029, THYMU1000033, THYMU1000041, THYMU1000060, THYMU1000074, THYMU1000098, THYMU1000105, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000331, THYMU1000336, THYMU1000366, THYMU1000382, THYMU1000393, THYMU1000394, THYMU1000399, THYMU1000426, THYMU1000440, THYMU1000459, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032,

THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000317, THYMU2000368, THYMU2000369, THYMU2000418, THYMU2000436, THYMU2000440, THYMU2000489, THYMU2000492, THYMU2000519, THYMU2000570, THYMU2000602, THYMU2000660, THYMU2000671, THYMU2000702, THYMU2000767, THYMU2000800, THYMU2000879, THYMU2000932, THYMU2000946, THYMU2000950, THYMU2000971, THYMU2000982, THYMU2000990, THYMU2001007, THYMU2001018, THYMU2001053, THYMU2001071, THYMU2001090, THYMU2001139, THYMU2001202, THYMU2001203, THYMU2001256, THYMU2001270, THYMU2001291, THYMU2001314, THYMU2001325, THYMU2001357, THYMU2001381, THYMU2001422,

THYMU2001443, THYMU2001515, THYMU2001521, THYMU2001556, THYMU2001622, THYMU2001727, THYMU2001825, THYMU2001868, THYMU2001898, THYMU2001900, THYMU2001916, THYMU2001926, THYMU2001989, THYMU2002023, THYMU2002037, THYMU2002080, THYMU2002109, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002789, THYMU2002792, THYMU2002815, THYMU2002841, THYMU2002852, THYMU2002910, THYMU2002950, THYMU2003069, THYMU2003107, THYMU2003124, THYMU2003166, THYMU2003232, THYMU2003242, THYMU2003249, THYMU2003270, THYMU2003282, THYMU2003336,

THYMU2003366, THYMU2003397, THYMU2003440, THYMU2003470, THYMU2003479, THYMU2003632, THYMU2003650, THYMU2003700, THYMU2003760, THYMU2003803, THYMU2003855, THYMU2003907, THYMU2004003, THYMU2004008, THYMU2004042, THYMU2004108, THYMU2004111, THYMU2004123, THYMU2004139, THYMU2004169, THYMU2004284, THYMU2004336, THYMU2004344, THYMU2004356, THYMU2004452, THYMU2004508, THYMU2004512, THYMU2004635, THYMU2004639, THYMU2004668, THYMU2004677, THYMU2004688, THYMU2004693, THYMU2004835, THYMU2004843, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321,

THYMU2005356, THYMU2005369, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006505, THYMU2006666, THYMU2006712, THYMU2006913, THYMU2006946, THYMU2006965, THYMU2007025, THYMU2007036, THYMU2007095, THYMU2007112, THYMU2007146, THYMU2007179, THYMU2007258, THYMU2007307, THYMU2007308, THYMU2007339, THYMU2007350, THYMU2007384, THYMU2007493,

THYMU2007521, THYMU2007528, THYMU2007530, THYMU2007532, THYMU2007561, THYMU2007571, THYMU2007635, THYMU2007658, THYMU2007667, THYMU2007725, THYMU2007729, THYMU2007802, THYMU2007824, THYMU2007854, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008339, THYMU2008350, THYMU2008351, THYMU2008383, THYMU2008452, THYMU2008643, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104,

THYMU2009134, THYMU2009157, THYMU2009181, THYMU2009338, THYMU2009425, THYMU2009516, THYMU2009526, THYMU2009546, THYMU2009592, THYMU2009596, THYMU2009835, THYMU2009906, THYMU2009948, THYMU2010030, THYMU2010041, THYMU2010046, THYMU2010059, THYMU2010082, THYMU2010094, THYMU2010161, THYMU2010192, THYMU2010637, THYMU2010671, THYMU2010699, THYMU2010705, THYMU2010779, THYMU2010816, THYMU2010831, THYMU2010848, THYMU2011053, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011538, THYMU2011548, THYMU2011551, THYMU2011564, THYMU2011573, THYMU2011585, THYMU2011621, THYMU2011666,

THYMU2011736, THYMU2011785, THYMU2011806, THYMU2011852, THYMU2011875, THYMU2011881, THYMU2011937, THYMU2011939, THYMU2012002, THYMU2012024, THYMU2012073, THYMU2012104, THYMU2012113, THYMU2012273, THYMU2012401, THYMU2012507, THYMU2012565, THYMU2012574, THYMU2012625, THYMU2012631, THYMU2012665, THYMU2012701, THYMU2012807, THYMU2012826, THYMU2012882, THYMU2012891, THYMU2012902, THYMU2012919, THYMU2013023, THYMU2013047, THYMU2013051, THYMU2013089, THYMU2013136, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014183, THYMU2014204, THYMU2014297,

THYMU2014323, THYMU2014327, THYMU2014353, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015775, THYMU2015825, THYMU2015918, THYMU2015943, THYMU2015984, THYMU2016113, THYMU2016164, THYMU2016204, THYMU2016219, THYMU2016360, THYMU2016496, THYMU2016523, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831,

THYMU2017844, THYMU2017878, THYMU2017948, THYMU2018028, THYMU2018071, THYMU2018189, THYMU2018197, THYMU2018384, THYMU2018455, THYMU2018547, THYMU2018565, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587, THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264,

THYMU2023576, THYMU2023711, THYMU2023900, THYMU2023943, THYMU2023967, THYMU2024071, THYMU2024185, THYMU2024207, THYMU2024616, THYMU2024684, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027996, THYMU2028041, THYMU2028368, THYMU2028379, THYMU2028394, THYMU2028412, THYMU2028632, THYMU2028724, THYMU2028739, THYMU2028942, THYMU2028978, THYMU2029134, THYMU2029676, THYMU2029688, THYMU2029788,

THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031890, THYMU2031994, THYMU2032035, THYMU2032080, THYMU2032150, THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033308, THYMU2033401, THYMU2033583, THYMU2033755, THYMU2033787, THYMU2033816, THYMU2034182, THYMU2034279, THYMU2034314,

THYMU2034338, THYMU2034917, THYMU2035064, THYMU2035078, THYMU2035101, THYMU2035319, THYMU2035321, THYMU2035388, THYMU2035400, THYMU2035554, THYMU2035710, THYMU2035735, THYMU2036058, THYMU2036105, THYMU2036207, THYMU2036215, THYMU2036252, THYMU2036265, THYMU2036366, THYMU2036459, THYMU2036461, THYMU2036604, THYMU2036653, THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634,

THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390, THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003092, THYMU3003212, THYMU3003309,

THYMU3003350, THYMU3003403, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371, THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755,

THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571, THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713,

THYMU3020856, THYMU3020869, THYMU3020970, THYMU3021404, THYMU3021586, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000, THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231,

THYMU3030706, THYMU3030710, THYMU3030752, THYMU3031146, THYMU3031402, THYMU3031610, THYMU3031612, THYMU3031868, THYMU3031878, THYMU3032032, THYMU3033402, THYMU3033626, THYMU3033630, THYMU3033649, THYMU3033754, THYMU3033759, THYMU3034078, THYMU3034099, THYMU3034453, THYMU3034616, THYMU3034671, THYMU3034853, THYMU3034867, THYMU3034983, THYMU3036200, THYMU3036310, THYMU3036934, THYMU3036953, THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970,

THYMU3039807, THYMU3039846, THYMU3040126, THYMU3040146, THYMU3040168, THYMU3040172, THYMU3040725, THYMU3040746, THYMU3040829, THYMU3040830, THYMU3040986, THYMU3041354, THYMU3041386, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779, THYMU3043883, THYMU3043993, THYMU3044175, THYMU3044188, THYMU3044441, THYMU3044445, THYMU3045673, THYMU3045692, THYMU3045704, THYMU3046140, THYMU3046350, THYMU3046360, THYMU3046856, THYMU3047144, THYMU3047156, THYMU3047366, THYMU3047513, THYMU3047542, THYMU3047760, THYMU3047891, TKIDN1000001, TKIDN1000044,

TKIDN1000062, TKIDN1000066, TKIDN1000164, TKIDN1000171, TKIDN1000192, TKIDN2000127, TKIDN2000240, TKIDN2000246, TKIDN2000319, TKIDN2000521, TKIDN2000701, TKIDN2001529, TKIDN2001662, TKIDN2002318, TKIDN2002329, TKIDN2002424, TKIDN2002632, TKIDN2003044, TKIDN2003059, TKIDN2003062, TKIDN2003078, TKIDN2003366, TKIDN2003396, TKIDN2003413, TKIDN2003597, TKIDN2003601, TKIDN2004185, TKIDN2004386, TKIDN2004458, TKIDN2004748, TKIDN2005721, TKIDN2005934, TKIDN2005947, TKIDN2005956, TKIDN2006525, TKIDN2006761, TKIDN2006852, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278,

TKIDN2010602, TKIDN2010812, TKIDN2010934, TKIDN2011051, TKIDN2011160, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015423, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002046, TLIVE2002336, TLIVE2002338, TLIVE2002562, TLIVE2002882, TLIVE2003197, TLIVE2003200, TLIVE2003225, TLIVE2003381, TLIVE2003737, TLIVE2003961, TLIVE2004110, TLIVE2004320,

TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000018, TRACH1000057, TRACH1000074, TRACH1000100, TRACH1000125, TRACH1000140, TRACH1000144,

TRACH1000181, TRACH1000193, TRACH1000205, TRACH1000212, TRACH1000240, TRACH2000002, TRACH2000015, TRACH2000024, TRACH2000032, TRACH2000079, TRACH2000081, TRACH2000083, TRACH2000148, TRACH2000185, TRACH2000196, TRACH2000235, TRACH2000250, TRACH2000259, TRACH2000272, TRACH2000287, TRACH2000289, TRACH2000306, TRACH2000312, TRACH2000336, TRACH2000393, TRACH2000411, TRACH2000420, TRACH2000461, TRACH2000496, TRACH2000497, TRACH2000502, TRACH2000522, TRACH2000559, TRACH2000660, TRACH2000665, TRACH2000675, TRACH2000677, TRACH2000703, TRACH2000767, TRACH2000775, TRACH2000780, TRACH2000807, TRACH2000862, TRACH2000873, TRACH2000894, TRACH2000898, TRACH2000923, TRACH2000926, TRACH2000944, TRACH2000959,

TRACH2000981, TRACH2000986, TRACH2001037, TRACH2001060, TRACH2001085, TRACH2001101, TRACH2001109, TRACH2001121, TRACH2001147, TRACH2001154, TRACH2001188, TRACH2001192, TRACH2001249, TRACH2001275, TRACH2001314, TRACH2001335, TRACH2001339, TRACH2001341, TRACH2001347, TRACH2001395, TRACH2001400, TRACH2001403, TRACH2001416, TRACH2001443, TRACH2001474, TRACH2001478, TRACH2001525, TRACH2001532, TRACH2001592, TRACH2001612, TRACH2001621, TRACH2001646, TRACH2001651, TRACH2001684, TRACH2001810, TRACH2001898, TRACH2001933, TRACH2001968, TRACH2001996, TRACH2002054, TRACH2002092, TRACH2002100, TRACH2002333, TRACH2002784, TRACH2002840, TRACH2002853, TRACH2002862, TRACH2002954, TRACH2002960, TRACH2002988,

TRACH2003070, TRACH2003272, TRACH2003484, TRACH2003516, TRACH2003605, TRACH2003736, TRACH2003759, TRACH2003941, TRACH2004039, TRACH2004054, TRACH2004087, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005800, TRACH2006015, TRACH2006049, TRACH2006097, TRACH2006194, TRACH2006327, TRACH2006331, TRACH2006378, TRACH2006387, TRACH2006528, TRACH2006670, TRACH2006762,

TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007754, TRACH2007894, TRACH2007969, TRACH2007988, TRACH2008081, TRACH2008112, TRACH2008113, TRACH2008127, TRACH2008130, TRACH2008278, TRACH2008300, TRACH2008583, TRACH2008597, TRACH2008820, TRACH2008891, TRACH2008921, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419,

TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011290, TRACH2011293, TRACH2011302, TRACH2011574, TRACH2011894, TRACH2011956, TRACH2012000, TRACH2012138, TRACH2012242, TRACH2012370, TRACH2012387, TRACH2012536, TRACH2012562, TRACH2012742, TRACH2012811, TRACH2012823, TRACH2012918, TRACH2013081, TRACH2013123, TRACH2013265, TRACH2013369, TRACH2013450, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014268,

TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016481, TRACH2016498, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949,

TRACH2017965, TRACH2017980, TRACH2018034, TRACH2018084, TRACH2018262, TRACH2018273, TRACH2018278, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2019024, TRACH2019046, TRACH2019080, TRACH2019151, TRACH2019154, TRACH2019176, TRACH2019248, TRACH2019309, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023299, TRACH2023306,

TRACH2023416, TRACH2023479, TRACH2023684, TRACH2023860, TRACH2023876, TRACH2024139, TRACH2024187, TRACH2024271, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025535, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926, TRACH3001119,

TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003379, TRACH3003586, TRACH3003683, TRACH3003780, TRACH3003805, TRACH3003832, TRACH3003872, TRACH3004068, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004721, TRACH3004747, TRACH3004760, TRACH3004783, TRACH3004786, TRACH3004840, TRACH3004931, TRACH3004934, TRACH3005072,

TRACH3005085, TRACH3005102, TRACH3005173, TRACH3005191, TRACH3005211, TRACH3005274, TRACH3005479, TRACH3005549, TRACH3005699, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006800, TRACH3006889, TRACH3006985, TRACH3007097, TRACH3007268, TRACH3007274, TRACH3007277, TRACH3007391, TRACH3007479, TRACH3007541, TRACH3007595, TRACH3007625, TRACH3007627, TRACH3007689, TRACH3007766, TRACH3007866, TRACH3007995, TRACH3008042, TRACH3008061, TRACH3008201, TRACH3008508, TRACH3008535, TRACH3008629,

TRACH3008632, TRACH3008688, TRACH3008697, TRACH3008713, TRACH3008887, TRACH3008902, TRACH3008990, TRACH3009001, TRACH3009059, TRACH3009061, TRACH3009148, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684,

TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447,

TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316,

TRACH3025346, TRACH3025520, TRACH3026148, TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678,

TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037267, TRACH3037288, TRACH3037505, TRACH3037573, TRACH3037696,

TRACH3037897, TRACH3038086, TRACH3038399, TSTOM1000135, TSTOM1000186, TSTOM2000139, TSTOM2000235, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000122, TUTER1000137, TUTER2000057, TUTER2000283, TUTER2000425, TUTER2000904, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000143, UMVEN1000156, UMVEN1000186, UMVEN2000046, UMVEN2000069, UMVEN2000121, UMVEN2000152, UMVEN2000354,

UTERU1000008, UTERU1000015, UTERU1000024, UTERU1000031, UTERU1000032, UTERU1000057, UTERU1000065, UTERU1000077, UTERU1000093, UTERU1000096, UTERU1000106, UTERU1000109, UTERU1000131, UTERU1000138, UTERU1000148, UTERU1000160, UTERU1000182, UTERU1000183, UTERU1000187, UTERU1000192, UTERU1000249, UTERU1000337, UTERU1000339, UTERU1000384, UTERU2000023, UTERU2000047, UTERU2000074, UTERU2000099, UTERU2000154, UTERU2000197, UTERU2000218, UTERU2000238, UTERU2000243, UTERU2000260, UTERU2000263, UTERU2000300, UTERU2000329, UTERU2000349, UTERU2000377, UTERU2000393, UTERU2000418, UTERU2000465, UTERU2000485, UTERU2000517, UTERU2000524, UTERU2000539, UTERU2000542, UTERU2000546, UTERU2000550, UTERU2000569,

UTERU2000607, UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001607, UTERU2001658, UTERU2001747, UTERU2001876, UTERU2002001, UTERU2002011, UTERU2002176, UTERU2002198, UTERU2002332, UTERU2002473, UTERU2002547, UTERU2002662, UTERU2002693, UTERU2002733, UTERU2002736, UTERU2002737, UTERU2002826, UTERU2002841, UTERU2002964, UTERU2002993, UTERU2003035, UTERU2003126, UTERU2003135, UTERU2003321, UTERU2003399, UTERU2003411, UTERU2003577, UTERU2003704, UTERU2003926, UTERU2003973, UTERU2004015, UTERU2004037,

UTERU2004039, UTERU2004061, UTERU2004073, UTERU2004163, UTERU2004299, UTERU2004461, UTERU2004520, UTERU2004564, UTERU2004688, UTERU2004698, UTERU2004861, UTERU2004929, UTERU2005004, UTERU2005050, UTERU2005069, UTERU2005074, UTERU2005179, UTERU2005346, UTERU2005446, UTERU2005449, UTERU2005450, UTERU2005533, UTERU2005601, UTERU2005664, UTERU2005822, UTERU2005903, UTERU2005905, UTERU2006103, UTERU2006137, UTERU2006182, UTERU2006400, UTERU2006412, UTERU2006429, UTERU2006486, UTERU2006524, UTERU2006547, UTERU2006568, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639,

UTERU2007724, UTERU2007924, UTERU2007942, UTERU2008018, UTERU2008019, UTERU2008027, UTERU2008040, UTERU2008077, UTERU2008130, UTERU2008347, UTERU2008426, UTERU2008516, UTERU2008561, UTERU2008653, UTERU2008705, UTERU2008707, UTERU2008747, UTERU2008785, UTERU2008845, UTERU2008901, UTERU2008930, UTERU2008938, UTERU2008962, UTERU2009094, UTERU2009120, UTERU2009131, UTERU2009147, UTERU2009206, UTERU2009283, UTERU2009335, UTERU2009414, UTERU2009435, UTERU2009483, UTERU2009510, UTERU2009540, UTERU2009776, UTERU2009904, UTERU2009951, UTERU2009972, UTERU2010115, UTERU2010124, UTERU2010164, UTERU2010231, UTERU2010304, UTERU2010320, UTERU2010431, UTERU2010525, UTERU2010651, UTERU2010724, UTERU2010747,

UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031, UTERU2012101, UTERU2012114, UTERU2012230, UTERU2012252, UTERU2012286, UTERU2012333, UTERU2012407, UTERU2012526, UTERU2012581, UTERU2012610, UTERU2012615, UTERU2012688, UTERU2012703, UTERU2012715, UTERU2012767, UTERU2012786, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001, UTERU2014024,

UTERU2014167, UTERU2014223, UTERU2014398, UTERU2014464, UTERU2014548, UTERU2014601, UTERU2014631, UTERU2014668, UTERU2014678, UTERU2014728, UTERU2014998, UTERU2015062, UTERU2015087, UTERU2015108, UTERU2015198, UTERU2015202, UTERU2015405, UTERU2015640, UTERU2015653, UTERU2015830, UTERU2015880, UTERU2016147, UTERU2016157, UTERU2016426, UTERU2016464, UTERU2016669, UTERU2016757, UTERU2016761, UTERU2016799, UTERU2016896, UTERU2016902, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522,

UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019005, UTERU2019038, UTERU2019096, UTERU2019163, UTERU2019257, UTERU2019453, UTERU2019491, UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023175, UTERU2023262, UTERU2023651, UTERU2023687, UTERU2023712, UTERU2023941, UTERU2024002, UTERU2024042,

UTERU2024141, UTERU2024481, UTERU2024656, UTERU2024758, UTERU2024820, UTERU2024881, UTERU2024969, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941, UTERU2028377, UTERU2028734, UTERU2029503, UTERU2029660, UTERU2029742, UTERU2029953, UTERU2030213, UTERU2030270, UTERU2030280, UTERU2031060, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420,

UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623, UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000844, UTERU3000899, UTERU3000959, UTERU3001029, UTERU3001053, UTERU3001059,

UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465, UTERU3003495, UTERU3003523, UTERU3003660, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005159, UTERU3005205, UTERU3005230, UTERU3005264, UTERU3005422, UTERU3005460, UTERU3005536, UTERU3005585, UTERU3005907,

UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010409, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599,

UTERU3012999, UTERU3013167, UTERU3013302, UTERU3013781, UTERU3014274, UTERU3014446, UTERU3014611, UTERU3014647, UTERU3014791, UTERU3014906, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the SYNOV, T1ESE, TBAES, TCERX, TCOLN, TESOP, and TESTI libraries were analyzed for expression frequency. The analysis results are described below. Each is a relative value, where the expression level over all tissues is taken as 100. The larger this value, the greater the level of expression.

TABLE 38

| Clone Name | SYNOV | T1ESE | TBAES | TCERX | TCOLN | TESOP | TESTI |
|---|---|---|---|---|---|---|---|
| 3NB691000116 | 0 | 0 | 0 | 0 | 34.479 | 0 | 0 |
| 3NB691000173 | 0 | 0 | 5.555 | 0 | 0 | 0 | 2.065 |
| 3NB692000281 | 0 | 4.889 | 0 | 4.646 | 0 | 6.024 | 0 |
| 3NB692000330 | 0.951 | 0 | 0 | 0 | 0 | 0 | 1.153 |
| 3NB692000374 | 0 | 0 | 0 | 0 | 0 | 0 | 0.709 |
| 3NB692000529 | 0 | 0 | 0 | 0 | 0 | 0 | 7.289 |
| 3NB692000545 | 0 | 0 | 0 | 0 | 0 | 0 | 1.844 |
| 3NB692000912 | 1.013 | 0 | 0 | 0 | 0 | 0 | 0 |
| 3NB692001334 | 0 | 0 | 3.777 | 0 | 0 | 0 | 0 |
| 3NB692001366 | 0 | 0 | 0 | 0 | 0 | 0 | 8.225 |
| 3NB692001442 | 0 | 26.830 | 0 | 0 | 0 | 0 | 0.790 |
| 3NB692001496 | 0 | 0 | 0 | 0 | 0 | 0 | 5.032 |
| 3NB692001511 | 0 | 0 | 0 | 0 | 0 | 0 | 5.628 |
| 3NB692001557 | 0 | 0 | 0 | 0 | 0 | 0 | 3.965 |
| 3NB692008178 | 0 | 0 | 0 | 0 | 0 | 0 | 8.225 |
| ADRGL1000018 | 0 | 0 | 0 | 0 | 0 | 0 | 3.161 |
| ADRGL1000065 | 0 | 0 | 0 | 0 | 0 | 0 | 7.373 |
| ADRGL2000006 | 0 | 0 | 0 | 0 | 0 | 0 | 2.039 |
| ADRGL2000042 | 0 | 0 | 0 | 0 | 0 | 0 | 4.980 |
| ADRGL2000097 | 0 | 0 | 15.639 | 0 | 0 | 0 | 0 |
| ADRGL2000117 | 0 | 0 | 0 | 0 | 0 | 0 | 5.579 |
| ADRGL2000328 | 0 | 0 | 0 | 0 | 0 | 0 | 4.401 |
| ADRGL2009273 | 2.996 | 0 | 0 | 0 | 0 | 0 | 1.816 |
| ASTRO2000014 | 0 | 9.121 | 0 | 0 | 0 | 0 | 0 |
| ASTRO2000046 | 0.880 | 0 | 0 | 0 | 0 | 0 | 0.533 |
| ASTRO2000141 | 0 | 0 | 0 | 0 | 32.199 | 0 | 0.993 |
| ASTRO2001029 | 2.705 | 0 | 0 | 0 | 26.586 | 0 | 0 |
| ASTRO2001076 | 3.066 | 0 | 0 | 0 | 0 | 0 | 0 |
| ASTRO2001806 | 0 | 0 | 0 | 0 | 0 | 0 | 5.611 |
| ASTRO2001823 | 0 | 0 | 0 | 0 | 0 | 0 | 1.898 |
| ASTRO2002024 | 0 | 0 | 0 | 0 | 0 | 0 | 8.570 |
| ASTRO2002459 | 0 | 0 | 0 | 0 | 0 | 5.989 | 0 |
| ASTRO2002720 | 0 | 0 | 0 | 0 | 0 | 0 | 15.873 |
| ASTRO2002842 | 0 | 0 | 67.001 | 0 | 0 | 0 | 0 |
| ASTRO2003024 | 0 | 0 | 0 | 0 | 0 | 0 | 1.859 |
| ASTRO2003212 | 0 | 0 | 0 | 0 | 0 | 0 | 9.792 |
| ASTRO2003316 | 0 | 0 | 0 | 0 | 0 | 20.183 | 3.857 |
| ASTRO2003632 | 0 | 0 | 0 | 0 | 8.808 | 0 | 0 |
| ASTRO2003840 | 0 | 0 | 0 | 0 | 0 | 0 | 8.098 |
| ASTRO2004628 | 19.197 | 0 | 0 | 0 | 0 | 0 | 0 |
| ASTRO2005360 | 0 | 0 | 0 | 0 | 0 | 40.816 | 3.900 |
| ASTRO2005687 | 2.404 | 0 | 0 | 0 | 0 | 0 | 0 |
| ASTRO2007047 | 4.507 | 0 | 0 | 0 | 0 | 0 | 0 |
| ASTRO2007948 | 0 | 0 | 0 | 0 | 0 | 17.135 | 0 |
| ASTRO2010819 | 2.903 | 0 | 0 | 0 | 0 | 0 | 7.477 |
| ASTRO2012552 | 0 | 0 | 0 | 0 | 0 | 10.113 | 0 |
| ASTRO2013585 | 0 | 0 | 0 | 0 | 0 | 0 | 1.912 |
| ASTRO2014576 | 0 | 0 | 0 | 0 | 0 | 0 | 7.388 |
| ASTRO2015529 | 0 | 0 | 0 | 0 | 0 | 0 | 7.267 |
| ASTRO2016114 | 0 | 0 | 0 | 0 | 0 | 0 | 9.476 |
| ASTRO2016491 | 2.172 | 0 | 3.542 | 0 | 0 | 3.445 | 1.975 |
| ASTRO2016819 | 0 | 0 | 45.393 | 0 | 0 | 0 | 4.218 |
| ASTRO2018169 | 2.037 | 0 | 0 | 0 | 0 | 0 | 0.617 |
| ASTRO2018540 | 0 | 34.540 | 0 | 0 | 0 | 0 | 0 |
| BGGI11000123 | 1.512 | 0 | 0 | 0 | 0 | 0 | 0.916 |
| BGGI11000193 | 6.438 | 0 | 0 | 0 | 0 | 0 | 0 |
| BGGI12000054 | 0 | 0 | 1.657 | 0 | 0 | 1.611 | 0 |
| BGGI12000616 | 0 | 0 | 0 | 18.869 | 0 | 0 | 0.584 |
| BGGI12000839 | 1.590 | 0 | 0 | 0 | 0 | 0 | 1.446 |
| BGGI12001075 | 0 | 0 | 0 | 0 | 0 | 0 | 2.588 |
| BLADE2002730 | 0 | 0 | 0 | 0 | 0 | 0 | 8.520 |
| BLADE2006830 | 0 | 0 | 0 | 0 | 0 | 0 | 1.099 |
| BNGH41000190 | 0 | 0 | 0 | 0 | 0 | 0 | 1.654 |
| BNGH41000198 | 4.481 | 0 | 0 | 0 | 0 | 0 | 0 |
| BNGH41000235 | 0 | 0 | 0 | 0 | 0 | 0 | 21.303 |
| BNGH42000360 | 0 | 0 | 0 | 0 | 3.335 | 0 | 0.103 |
| BNGH42000739 | 0 | 0 | 0 | 0 | 0 | 0 | 0.407 |
| BNGH42001247 | 0 | 0 | 0 | 0 | 0 | 0 | 3.439 |
| BNGH42001406 | 0 | 0 | 0 | 0 | 0 | 47.011 | 0 |
| BNGH42001576 | 0 | 0 | 0 | 0 | 0 | 0 | 12.758 |
| BNGH42002387 | 0 | 0 | 0 | 0 | 0 | 0 | 6.899 |
| BNGH42004679 | 0 | 0 | 0 | 0 | 0 | 0 | 7.814 |
| BNGH42005968 | 0 | 0 | 0 | 0 | 0 | 0 | 1.501 |
| BNGH42006135 | 1.424 | 0 | 0 | 0 | 13.997 | 0 | 3.884 |
| BNGH42007037 | 0 | 0 | 0 | 0 | 0 | 0 | 4.066 |
| BNGH42007460 | 2.173 | 0 | 0 | 0 | 0 | 0 | 3.952 |
| BNGH42007709 | 0.496 | 0 | 0 | 0 | 0 | 0 | 0.150 |
| BNGH42007798 | 0 | 0 | 0 | 0 | 0 | 0 | 6.420 |
| BNGH42008743 | 3.320 | 34.193 | 0 | 0 | 0 | 0 | 2.013 |
| BNGH42009025 | 0 | 0 | 0 | 0 | 29.557 | 0 | 1.823 |
| BRACE1000073 | 0 | 0 | 0 | 0 | 0 | 0 | 2.756 |
| BRACE1000115 | 0 | 0 | 0 | 3.950 | 0 | 0 | 0.245 |
| BRACE1000131 | 0 | 0 | 7.327 | 0 | 0 | 0 | 1.362 |
| BRACE1000159 | 0 | 0 | 0 | 38.966 | 0 | 0 | 0 |
| BRACE2000077 | 0 | 0 | 0 | 0 | 0 | 0 | 31.530 |
| BRACE2000183 | 0 | 0 | 0 | 96.746 | 0 | 0 | 0 |
| BRACE2000229 | 0 | 0 | 90.835 | 0 | 0 | 0 | 0 |
| BRACE2000347 | 0 | 0 | 18.185 | 0 | 0 | 0 | 3.380 |
| BRACE2000577 | 0 | 0 | 0 | 0 | 0 | 0 | 47.943 |
| BRACE2000885 | 0 | 0 | 0 | 0 | 0 | 0 | 47.943 |
| BRACE2000893 | 0 | 0 | 0 | 0 | 0 | 0 | 2.954 |
| BRACE2000975 | 0 | 0 | 0 | 0 | 0 | 0 | 3.350 |
| BRACE2001107 | 0 | 0 | 0 | 0 | 0 | 0 | 9.621 |
| BRACE2001117 | 0 | 0 | 19.348 | 0 | 0 | 0 | 0 |
| BRACE2001143 | 0 | 0 | 0 | 62.699 | 0 | 0 | 1.942 |
| BRACE2001154 | 0 | 0 | 0 | 0 | 0 | 0 | 6.945 |
| BRACE2001477 | 0 | 0 | 0 | 0 | 0 | 0 | 2.248 |
| BRACE2001497 | 0 | 0 | 0 | 0 | 0 | 12.749 | 0 |
| BRACE2001564 | 0 | 0 | 0 | 0 | 0 | 0 | 31.530 |
| BRACE2001692 | 0 | 0 | 0 | 12.779 | 0 | 0 | 0.396 |
| BRACE2001711 | 0 | 0 | 27.364 | 0 | 0 | 0 | 0 |
| BRACE2001892 | 0 | 0 | 0 | 0 | 0 | 0 | 1.188 |
| BRACE2002050 | 0 | 0 | 0 | 0 | 0 | 0 | 3.791 |
| BRACE2002091 | 0 | 0 | 0 | 0 | 0 | 0 | 6.902 |
| BRACE2002202 | 0 | 0 | 0 | 0 | 0 | 0 | 23.489 |
| BRACE2002356 | 0 | 0 | 0 | 0 | 0 | 0 | 11.877 |
| BRACE2002392 | 0 | 0 | 0 | 0 | 0 | 0 | 1.127 |
| BRACE2002441 | 2.429 | 0 | 0 | 0 | 0 | 0 | 0 |
| BRACE2002649 | 0 | 0 | 0 | 0 | 0 | 0 | 6.921 |
| BRACE2002661 | 0 | 0 | 0 | 0 | 0 | 19.704 | 1.883 |
| BRACE2002677 | 0 | 0 | 0 | 0 | 0 | 0 | 1.262 |
| BRACE2002807 | 0 | 0 | 0 | 0 | 0 | 0 | 21.066 |
| BRACE2002861 | 0 | 0 | 0 | 0 | 55.019 | 0 | 1.696 |
| BRACE2003172 | 2.804 | 0 | 0 | 6.859 | 0 | 0 | 0 |
| BRACE2003527 | 0 | 0 | 0 | 0 | 0 | 0 | 1.685 |
| BRACE2003628 | 0 | 0 | 0 | 0 | 0 | 0 | 4.460 |
| BRACE2003827 | 0 | 0 | 0 | 0 | 0 | 0 | 5.154 |
| BRACE2003846 | 0 | 0 | 0 | 0 | 0 | 4.459 | 0 |
| BRACE2003892 | 4.707 | 0 | 0 | 0 | 0 | 14.932 | 0 |
| BRACE2003904 | 0 | 0 | 18.237 | 0 | 0 | 0 | 0 |
| BRACE2005087 | 0 | 0 | 0 | 0 | 0 | 0 | 3.390 |
| BRACE2005090 | 0 | 0 | 0 | 0 | 0 | 0 | 47.943 |
| BRACE2005151 | 4.532 | 0 | 0 | 0 | 0 | 0 | 0 |
| BRACE2005231 | 0 | 0 | 0 | 0 | 0 | 0 | 2.087 |
| BRACE2005433 | 0 | 0 | 0 | 0 | 0 | 0 | 7.549 |
| BRACE2005434 | 1.487 | 0 | 0 | 0 | 0 | 0 | 2.704 |
| BRACE2006072 | 0 | 0 | 0 | 0 | 0 | 0 | 12.884 |
| BRACE2006105 | 0 | 0 | 0 | 0 | 0 | 0 | 27.736 |
| BRACE2006319 | 0 | 0 | 0 | 0 | 0 | 0 | 11.459 |
| BRACE2006397 | 1.273 | 0 | 0 | 6.229 | 12.516 | 0 | 0 |
| BRACE2006514 | 0 | 0 | 0 | 0 | 0 | 0 | 0.568 |
| BRACE2006547 | 0 | 0 | 0 | 0 | 0 | 0 | 19.561 |
| BRACE2006859 | 0 | 0 | 0 | 0 | 0 | 0 | 6.642 |
| BRACE2006900 | 0 | 0 | 10.859 | 10.859 | 21.818 | 0 | 0.673 |
| BRACE2007064 | 0 | 0 | 0 | 0 | 0 | 11.117 | 0 |
| BRACE2007089 | 0 | 0 | 0 | 0 | 0 | 0 | 0.553 |
| BRACE2007109 | 0 | 0 | 0 | 0 | 0 | 0 | 0.624 |
| BRACE2007191 | 0 | 0 | 4.832 | 0 | 0 | 0 | 0.898 |
| BRACE2007401 | 0 | 0 | 0 | 0 | 38.746 | 0 | 2.389 |
| BRACE2007518 | 4.895 | 0 | 0 | 0 | 0 | 0 | 0 |
| BRACE2007567 | 2.993 | 0 | 0 | 0 | 0 | 0 | 0.907 |
| BRACE2007708 | 0 | 0 | 0 | 0 | 45.054 | 0 | 1.389 |
| BRACE2007801 | 0 | 0 | 0 | 0 | 0 | 0 | 47.943 |
| BRACE2008358 | 0 | 0 | 0 | 0 | 0 | 15.504 | 1.481 |
| BRACE2008385 | 0 | 0 | 0 | 0 | 0 | 0 | 1.365 |
| BRACE2008399 | 0 | 0 | 0 | 0 | 0 | 37.868 | 0 |
| BRACE2008402 | 0 | 0 | 0 | 0 | 0 | 28.691 | 2.741 |
| BRACE2008481 | 6.021 | 0 | 0 | 0 | 0 | 0 | 0 |
| BRACE2008653 | 0 | 0 | 0 | 0 | 0 | 0 | 2.413 |
| BRACE2008706 | 0 | 0 | 0 | 0 | 0 | 0 | 2.148 |
| BRACE2008968 | 0 | 0 | 4.090 | 0 | 0 | 0 | 0.380 |
| BRACE2009037 | 0 | 0 | 0 | 0 | 0 | 0 | 0.764 |
| BRACE2009044 | 0 | 0 | 0 | 0 | 0 | 0 | 13.117 |
| BRACE2009045 | 0 | 0 | 0 | 0 | 0 | 0 | 3.936 |
| BRACE2009291 | 0 | 0 | 0 | 0 | 0 | 0 | 0.865 |
| BRACE2009307 | 0 | 0 | 0 | 0 | 0 | 0 | 29.044 |
| BRACE2009517 | 5.850 | 0 | 0 | 0 | 0 | 0 | 0 |
| BRACE2009558 | 0 | 0 | 0 | 0 | 0 | 0 | 1.880 |
| BRACE2010669 | 0 | 0 | 0 | 0 | 0 | 0 | 4.167 |
| BRACE2012528 | 0 | 0 | 0 | 0 | 0 | 0 | 0.310 |
| BRACE2012814 | 0 | 0 | 0 | 0 | 0 | 0 | 1.002 |
| BRACE2015287 | 0 | 0 | 0 | 0 | 0 | 0 | 1.501 |
| BRACE2016362 | 0 | 0 | 0 | 0 | 0 | 0 | 10.077 |
| BRACE2017397 | 0 | 0 | 0 | 0 | 0 | 0 | 1.542 |
| BRACE2017574 | 0 | 0 | 36.041 | 0 | 0 | 0 | 0 |
| BRACE2018736 | 0 | 0 | 0 | 0 | 0 | 0 | 26.439 |
| BRACE2018847 | 0 | 0 | 0 | 0 | 0 | 0 | 25.504 |
| BRACE2021014 | 0 | 0 | 0 | 0 | 0 | 0 | 11.618 |
| BRACE2021143 | 0 | 0 | 55.669 | 0 | 0 | 0 | 5.173 |
| BRACE2022333 | 0 | 0 | 0 | 0 | 0 | 0 | 5.803 |
| BRACE2022389 | 0 | 0 | 0 | 0 | 0 | 0 | 6.946 |
| BRACE2022638 | 1.608 | 0 | 0 | 0 | 0 | 5.100 | 1.462 |
| BRACE2023718 | 0 | 0 | 0 | 0 | 0 | 0 | 10.491 |
| BRACE2023727 | 0 | 0 | 0 | 0 | 0 | 0 | 12.906 |
| BRACE2024627 | 0 | 0 | 0 | 0 | 0 | 0 | 8.002 |
| BRACE2025437 | 0 | 0 | 0 | 0 | 0 | 0 | 3.991 |
| BRACE2026131 | 0 | 0 | 0 | 0 | 0 | 0 | 47.943 |
| BRACE2030341 | 0 | 0 | 0 | 0 | 0 | 47.323 | 0 |
| BRACE2032477 | 0 | 0 | 0 | 0 | 0 | 38.127 | 0 |
| BRACE2038269 | 0 | 0 | 0 | 0 | 0 | 0 | 47.943 |
| BRACE2039624 | 4.173 | 0 | 0 | 0 | 0 | 0 | 1.265 |
| BRACE2043142 | 0 | 0 | 0 | 0 | 0 | 0 | 47.943 |
| BRACE2043381 | 0 | 0 | 0 | 0 | 0 | 73.638 | 0 |
| BRACE2044105 | 0 | 0 | 0 | 0 | 0 | 0 | 13.155 |
| BRACE3000973 | 0 | 0 | 0 | 0 | 0 | 0 | 35.242 |
| BRACE3004150 | 0 | 0 | 0 | 0 | 0 | 0 | 2.539 |
| BRACE3004205 | 0 | 0 | 0 | 0 | 0 | 0 | 31.530 |
| BRACE3004843 | 0 | 0 | 0 | 0 | 0 | 0 | 20.797 |
| BRACE3005499 | 0 | 0 | 0 | 0 | 0 | 0 | 20.445 |
| BRACE3005938 | 2.375 | 0 | 0 | 0 | 0 | 7.535 | 0.720 |
| BRACE3006553 | 0 | 0 | 0 | 0 | 0 | 0 | 67.311 |
| BRACE3007058 | 0 | 0 | 0 | 0 | 0 | 0 | 47.943 |
| BRACE3009237 | 0 | 0 | 0 | 0 | 0 | 0 | 11.857 |
| BRACE3009416 | 0 | 0 | 0 | 0 | 0 | 0 | 18.656 |
| BRACE3010428 | 0 | 0 | 0 | 0 | 0 | 0 | 0.748 |
| BRACE3010438 | 0 | 39.772 | 0 | 0 | 0 | 0 | 0 |
| BRACE3016884 | 0 | 0 | 90.835 | 0 | 0 | 0 | 0 |
| BRACE3019941 | 0 | 0 | 0 | 0 | 0 | 0 | 15.278 |
| BRACE3021430 | 0 | 0 | 0 | 0 | 0 | 0 | 7.798 |
| BRACE3028895 | 0 | 0 | 0 | 0 | 0 | 0 | 29.749 |
| BRACE3045981 | 0 | 0 | 0 | 0 | 0 | 0 | 17.280 |
| BRALZ2018834 | 0 | 0 | 0 | 0 | 0 | 42.693 | 0 |
| BRAMY2000354 | 0 | 0 | 0 | 0 | 0 | 0 | 0.898 |
| BRAMY2000585 | 0 | 0 | 0 | 0 | 0 | 16.804 | 0 |
| BRAMY2000893 | 0 | 0 | 0 | 0 | 0 | 0 | 0.995 |
| BRAMY2003008 | 0 | 0 | 0 | 0 | 0 | 0 | 17.287 |
| BRAMY2004363 | 4.409 | 0 | 0 | 0 | 0 | 0 | 6.682 |
| BRAMY2005052 | 0 | 0 | 0 | 0 | 0 | 0 | 7.591 |
| BRAMY2005488 | 0 | 0 | 0 | 0 | 0 | 0 | 7.449 |
| BRAMY2006397 | 0 | 0 | 0 | 59.264 | 0 | 0 | 0 |
| BRAMY2007078 | 8.202 | 0 | 0 | 0 | 0 | 0 | 0 |
| BRAMY2007287 | 0 | 0 | 0 | 0 | 0 | 0 | 7.004 |
| BRAMY2008977 | 10.905 | 0 | 0 | 0 | 0 | 0 | 0 |
| BRAMY2009013 | 0 | 0 | 0 | 0 | 0 | 0 | 3.981 |
| BRAMY2009934 | 0 | 0 | 0 | 0 | 0 | 0 | 3.864 |
| BRAMY2011841 | 0 | 0 | 77.624 | 0 | 0 | 0 | 0 |
| BRAMY2012163 | 0 | 0 | 0 | 0 | 0 | 0 | 5.168 |
| BRAMY2012426 | 0 | 0 | 0 | 0 | 0 | 0 | 4.040 |
| BRAMY2012536 | 0 | 0 | 0 | 24.362 | 0 | 0 | 0.377 |
| BRAMY2013756 | 0 | 0 | 0 | 0 | 0 | 0 | 12.344 |
| BRAMY2015833 | 0 | 0 | 0 | 0 | 0 | 0 | 3.013 |
| BRAMY2016325 | 0 | 0 | 26.506 | 0 | 0 | 0 | 0 |
| BRAMY2017864 | 0 | 0 | 0 | 0 | 0 | 0 | 9.032 |
| BRAMY2018635 | 0 | 0 | 0 | 0 | 0 | 0 | 17.756 |
| BRAMY2019468 | 0 | 0 | 0 | 0 | 0 | 0 | 4.966 |
| BRAMY2019554 | 0 | 0 | 0 | 0 | 0 | 5.460 | 1.043 |
| BRAMY2019643 | 0 | 0 | 0 | 0 | 0 | 0 | 39.348 |
| BRAMY2020354 | 0 | 0 | 0 | 0 | 0 | 0 | 39.348 |
| BRAMY2020427 | 0 | 0 | 0 | 0 | 0 | 4.051 | 0.387 |
| BRAMY2020713 | 55.672 | 0 | 0 | 0 | 0 | 0 | 0 |
| BRAMY2021142 | 0 | 0 | 0 | 0 | 0 | 0 | 35.008 |
| BRAMY2023939 | 0 | 0 | 0 | 0 | 0 | 0 | 7.991 |
| BRAMY2024715 | 0 | 0 | 0 | 0 | 0 | 87.164 | 0 |
| BRAMY2026685 | 16.036 | 0 | 0 | 0 | 0 | 16.957 | 0 |
| BRAMY2028740 | 0 | 0 | 0 | 0 | 0 | 0 | 0.609 |
| BRAMY2028796 | 0 | 0 | 0 | 0 | 0 | 0 | 0.972 |
| BRAMY2029988 | 0 | 0 | 0 | 0 | 0 | 0 | 7.700 |
| BRAMY2030098 | 0 | 0 | 0 | 0 | 0 | 0 | 27.569 |
| BRAMY2032087 | 0 | 0 | 0 | 0 | 0 | 0 | 1.583 |
| BRAMY2032317 | 0 | 0 | 0 | 0 | 0 | 0 | 39.348 |
| BRAMY2034187 | 0 | 0 | 87.471 | 0 | 0 | 0 | 0 |
| BRAMY2035869 | 0 | 0 | 0 | 0 | 0 | 0 | 39.348 |
| BRAMY2036459 | 0 | 0 | 0 | 39.773 | 0 | 0 | 0 |
| BRAMY2037823 | 0 | 0 | 0 | 0 | 0 | 0 | 19.391 |
| BRAMY2038904 | 0 | 0 | 0 | 0 | 0 | 0 | 39.348 |
| BRAMY2040095 | 0 | 0 | 2.405 | 0 | 0 | 0 | 0.223 |
| BRAMY2040592 | 7.510 | 0 | 0 | 0 | 0 | 0 | 2.276 |
| BRAMY2047420 | 0 | 0 | 0 | 21.378 | 0 | 0 | 0 |
| BRAMY3002458 | 0 | 0 | 0 | 0 | 0 | 0 | 8.115 |
| BRAMY3003205 | 0 | 0 | 0 | 0 | 0 | 0 | 31.454 |
| BRAMY3004364 | 0 | 0 | 0 | 0 | 0 | 46.478 | 0 |
| BRAMY3004377 | 0 | 0 | 0 | 0 | 0 | 0 | 2.711 |
| BRAMY3008044 | 3.869 | 0 | 0 | 0 | 0 | 0 | 0 |
| BRAMY3009491 | 0 | 0 | 0 | 0 | 0 | 0 | 20.396 |
| BRAMY4000089 | 0 | 0 | 0 | 0 | 0 | 0 | 13.306 |
| BRAWH1000037 | 3.933 | 0 | 0 | 0 | 0 | 0 | 1.192 |
| BRAWH1000040 | 0 | 0 | 4.840 | 0 | 0 | 0 | 0 |
| BRAWH1000083 | 9.772 | 0 | 0 | 0 | 0 | 0 | 2.962 |
| BRAWH1000093 | 1.990 | 0 | 0 | 0 | 0 | 0 | 0 |
| BRAWH2000460 | 1.316 | 0 | 0 | 0 | 0 | 0 | 2.793 |
| BRAWH2000636 | 0 | 0 | 0 | 0 | 0 | 0 | 0.691 |
| BRAWH2000686 | 5.273 | 0 | 0 | 0 | 0 | 0 | 1.598 |
| BRAWH2000697 | 0 | 0 | 7.116 | 0 | 0 | 0 | 0.661 |
| BRAWH2000839 | 0 | 0 | 0 | 0 | 0 | 0 | 0.268 |
| BRAWH2000924 | 0 | 6.374 | 0 | 0 | 0 | 0 | 0 |
| BRAWH2000944 | 0 | 0 | 0 | 0 | 0 | 35.693 | 17.051 |
| BRAWH2001092 | 0.379 | 0 | 1.236 | 0 | 3.726 | 0 | 0.230 |
| BRAWH2001166 | 0 | 0 | 0 | 0 | 0 | 0 | 8.254 |
| BRAWH2001171 | 1.332 | 0 | 0 | 0 | 0 | 0 | 0 |
| BRAWH2001321 | 0 | 0 | 0 | 0 | 0 | 0 | 0.561 |
| BRAWH2001406 | 0 | 0 | 0 | 0 | 0 | 0 | 2.213 |
| BRAWH2001439 | 2.219 | 0 | 0 | 0 | 0 | 0 | 0.673 |
| BRAWH2001461 | 0 | 0 | 0 | 0 | 0 | 0 | 2.324 |
| BRAWH2001940 | 0 | 0 | 0 | 0 | 0 | 0 | 24.447 |
| BRAWH2001973 | 0 | 0 | 0 | 0 | 0 | 0 | 24.447 |
| BRAWH2002601 | 0 | 0 | 0 | 0 | 0 | 0 | 6.502 |
| BRAWH2003025 | 6.540 | 0 | 0 | 0 | 0 | 0 | 0 |
| BRAWH2003818 | 0 | 0 | 21.291 | 0 | 0 | 0 | 0 |
| BRAWH2004095 | 0 | 0 | 0 | 0 | 0 | 0 | 0.773 |
| BRAWH2004136 | 34.318 | 0 | 0 | 0 | 0 | 0 | 0 |
| BRAWH2005068 | 0 | 0 | 0 | 0 | 0 | 0 | 0.263 |
| BRAWH2005661 | 0 | 0 | 0 | 0 | 0 | 10.938 | 0 |
| BRAWH2006044 | 37.947 | 0 | 0 | 0 | 0 | 0 | 0 |
| BRAWH2006395 | 0 | 0 | 87.554 | 0 | 0 | 0 | 0 |
| BRAWH2006405 | 27.990 | 0 | 0 | 0 | 0 | 0 | 0 |
| BRAWH2006450 | 10.830 | 0 | 0 | 0 | 0 | 0 | 0 |
| BRAWH2006493 | 10.970 | 0 | 0 | 0 | 0 | 0 | 0 |
| BRAWH2006996 | 0 | 0 | 23.927 | 0 | 0 | 0 | 0 |
| BRAWH2007605 | 0 | 0 | 0 | 0 | 0 | 0 | 4.022 |
| BRAWH2008219 | 0 | 0 | 0 | 0 | 0 | 0 | 9.419 |
| BRAWH2008366 | 0 | 0 | 0 | 0 | 0 | 87.248 | 0 |
| BRAWH2008706 | 6.202 | 0 | 0 | 0 | 0 | 0 | 0 |
| BRAWH2009238 | 0 | 0 | 0 | 0 | 0 | 0 | 3.408 |
| BRAWH2009307 | 0 | 0 | 14.006 | 0 | 0 | 0 | 0 |
| BRAWH2010364 | 0 | 0 | 0 | 0 | 0 | 0 | 11.264 |
| BRAWH2010980 | 0 | 0 | 49.709 | 0 | 0 | 0 | 0 |
| BRAWH2012077 | 0 | 0 | 0 | 0 | 0 | 0 | 8.190 |
| BRAWH2014053 | 0 | 0 | 0 | 0 | 0 | 0 | 15.180 |
| BRAWH2014188 | 3.088 | 0 | 0 | 0 | 0 | 0 | 0.936 |
| BRAWH2014645 | 0 | 0 | 0 | 0 | 0 | 0 | 0.691 |
| BRAWH2015247 | 0 | 0 | 0 | 0 | 0 | 0 | 6.824 |
| BRAWH2016269 | 0 | 0 | 0 | 0 | 27.951 | 0 | 3.447 |
| BRAWH2016305 | 0 | 95.692 | 0 | 0 | 0 | 0 | 0 |
| BRAWH2016446 | 0 | 0 | 0 | 0 | 0 | 59.524 | 0 |
| BRAWH2017685 | 1.095 | 0 | 0 | 0 | 0 | 0 | 3.317 |
| BRAWH2018526 | 0 | 0 | 2.165 | 0 | 6.525 | 0 | 0.201 |
| BRAWH2018729 | 0 | 0 | 0 | 0 | 0 | 0 | 1.948 |
| BRAWH3001891 | 0 | 0 | 0 | 0 | 0 | 0 | 11.289 |
| BRAWH3002853 | 1.595 | 0 | 5.203 | 0 | 0 | 0 | 0.484 |
| BRAWH3003411 | 0 | 0 | 0 | 0 | 0 | 0 | 15.127 |
| BRAWH3003573 | 0 | 0 | 0 | 0 | 0 | 0 | 26.359 |
| BRAWH3004350 | 0 | 0 | 0 | 0 | 0 | 0 | 10.838 |
| BRAWH3006761 | 0 | 0 | 0 | 0 | 0 | 0 | 13.903 |
| BRAWH3018063 | 0 | 0 | 0 | 0 | 0 | 0 | 5.176 |
| BRAWH3021545 | 0 | 0 | 0 | 0 | 0 | 0 | 11.924 |
| BRAWH3021574 | 0 | 0 | 0 | 0 | 0 | 0 | 6.144 |
| BRAWH3024989 | 0 | 0 | 0 | 0 | 0 | 0 | 4.646 |
| BRAWH3027533 | 0 | 0 | 0 | 0 | 0 | 75.317 | 0 |
| BRAWH3029806 | 0 | 0 | 0 | 0 | 0 | 0 | 11.651 |
| BRCAN1000168 | 0 | 0 | 0 | 0 | 0 | 0 | 0.922 |
| BRCAN2003944 | 0 | 6.255 | 0 | 0 | 5.971 | 1.927 | 0 |
| BRCAN2004653 | 0 | 0 | 0 | 0 | 0 | 0 | 22.174 |
| BRCAN2007409 | 0 | 0 | 0 | 63.318 | 0 | 0 | 0 |
| BRCAN2011946 | 13.639 | 0 | 0 | 0 | 0 | 0 | 4.134 |
| BRCAN2020234 | 0 | 0 | 0 | 0 | 0 | 0 | 16.113 |
| BRCAN2025093 | 0 | 0 | 0 | 0 | 0 | 0 | 28.411 |
| BRCAN2026197 | 3.367 | 0 | 0 | 0 | 0 | 0 | 1.021 |
| BRCAN2027364 | 0 | 0 | 0 | 0 | 0 | 0 | 1.941 |
| BRCAN2028040 | 0 | 0 | 0 | 0 | 0 | 0 | 17.285 |
| BRCOC1000040 | 0 | 0 | 20.746 | 0 | 0 | 0 | 3.856 |
| BRCOC2003125 | 0 | 0 | 39.975 | 0 | 0 | 0 | 0 |
| BRCOC2003187 | 1.696 | 0 | 0 | 0 | 0 | 0 | 0 |
| BRCOC2005903 | 0 | 0 | 0 | 0 | 0 | 60.058 | 0 |
| BRCOC2006189 | 0 | 0 | 0 | 0 | 0 | 0 | 2.170 |
| BRCOC2009937 | 0 | 0 | 0 | 0 | 0 | 0 | 10.502 |
| BRCOC2010123 | 4.726 | 0 | 0 | 0 | 0 | 0 | 0 |
| BRCOC2012172 | 0 | 0 | 0 | 0 | 0 | 0 | 18.387 |
| BRHIP1000104 | 0 | 0 | 0 | 0 | 0 | 0 | 2.136 |
| BRHIP1000174 | 0 | 0 | 0 | 0 | 0 | 0 | 3.314 |
| BRHIP2000163 | 0 | 0 | 0 | 0 | 0 | 0 | 24.447 |
| BRHIP2000534 | 0 | 0 | 2.408 | 0 | 0 | 9.367 | 0.224 |
| BRHIP2000553 | 0 | 0 | 0 | 0 | 0 | 0 | 5.039 |
| BRHIP2001074 | 0 | 0 | 0 | 0 | 0 | 0 | 12.807 |
| BRHIP2003917 | 0 | 0 | 0 | 0 | 0 | 80.343 | 7.676 |
| BRHIP2007305 | 0 | 25.144 | 0 | 0 | 0 | 0 | 1.480 |
| BRHIP2011491 | 0 | 0 | 0 | 0 | 17.524 | 0 | 1.081 |
| BRHIP2011891 | 0 | 0 | 0 | 0 | 0 | 0 | 39.051 |
| BRHIP2013958 | 0 | 0 | 0 | 0 | 0 | 0 | 23.489 |
| BRHIP2014228 | 0 | 0 | 0 | 0 | 0 | 0 | 9.622 |
| BRHIP2015153 | 0 | 0 | 0 | 0 | 0 | 0 | 6.549 |
| BRHIP2015679 | 0 | 0 | 17.647 | 0 | 0 | 0 | 3.280 |
| BRHIP2017010 | 0 | 0 | 0 | 0 | 0 | 0 | 8.986 |
| BRHIP2018635 | 0 | 0 | 0 | 0 | 0 | 0 | 39.051 |
| BRHIP2018712 | 0 | 0 | 0 | 0 | 0 | 13.856 | 1.324 |
| BRHIP2019149 | 0 | 0 | 0 | 0 | 0 | 75.561 | 0 |
| BRHIP2021762 | 0 | 0 | 19.598 | 0 | 0 | 0 | 0 |
| BRHIP2021858 | 0 | 0 | 0 | 70.621 | 0 | 0 | 0 |
| BRHIP2021870 | 19.152 | 0 | 0 | 0 | 0 | 0 | 2.902 |
| BRHIP2022326 | 1.835 | 0 | 0 | 0 | 0 | 0 | 1.112 |
| BRHIP2023762 | 0 | 53.006 | 0 | 0 | 0 | 0 | 0 |
| BRHIP2023869 | 0 | 0 | 0 | 0 | 0 | 0 | 23.489 |
| BRHIP2023888 | 0.794 | 0 | 0 | 0 | 0 | 0 | 2.405 |
| BRHIP2024742 | 0 | 0 | 17.547 | 0 | 0 | 0 | 4.076 |
| BRHIP2024911 | 0 | 0 | 0 | 0 | 0 | 0 | 12.117 |
| BRHIP2027077 | 4.356 | 0 | 0 | 0 | 0 | 0 | 0 |
| BRHIP3000111 | 2.208 | 0 | 7.202 | 0 | 0 | 0 | 0 |
| BRHIP3001481 | 0 | 0 | 0 | 0 | 0 | 0 | 61.565 |
| BRHIP3003306 | 0 | 0 | 0 | 0 | 0 | 0 | 39.051 |
| BRHIP3003961 | 0 | 0 | 0 | 0 | 0 | 0 | 12.398 |
| BRHIP3005037 | 0 | 0 | 0 | 0 | 0 | 0 | 17.599 |
| BRHIP3005142 | 0 | 0 | 0 | 0 | 0 | 0 | 39.051 |
| BRHIP3006294 | 0 | 0 | 0 | 0 | 0 | 0 | 26.066 |
| BRHIP3007183 | 0 | 0 | 0 | 0 | 0 | 0 | 8.961 |
| BRHIP3007195 | 0 | 0 | 0 | 0 | 0 | 0 | 24.263 |
| BRHIP3007223 | 4.349 | 0 | 0 | 0 | 0 | 0 | 3.954 |
| BRHIP3012997 | 0 | 0 | 0 | 0 | 0 | 0 | 0.606 |
| BRHIP3013588 | 0 | 0 | 0 | 0 | 0 | 0 | 39.051 |
| BRHIP3017855 | 0 | 0 | 0 | 0 | 0 | 0 | 7.509 |
| BRHIP3020046 | 2.185 | 0 | 0 | 0 | 0 | 0 | 0 |
| BRSSN2000498 | 2.360 | 0 | 0 | 0 | 0 | 0 | 0.715 |
| BRSSN2000566 | 0 | 0 | 0 | 0 | 0 | 0 | 3.180 |
| BRSSN2000684 | 0 | 0 | 0 | 0 | 0 | 0 | 12.254 |
| BRSSN2001869 | 0 | 0 | 0 | 0 | 0 | 0 | 15.785 |
| BRSSN2004657 | 0 | 0 | 0 | 0 | 0 | 0 | 14.939 |
| BRSSN2009518 | 3.715 | 0 | 0 | 0 | 0 | 0 | 4.504 |
| BRSSN2013696 | 0 | 0 | 0 | 0 | 0 | 0 | 25.682 |
| BRSSN2013702 | 20.460 | 0 | 0 | 0 | 0 | 0 | 0 |
| BRSSN2014299 | 0 | 0 | 0 | 0 | 0 | 0 | 2.415 |
| BRSSN2014610 | 7.700 | 0 | 0 | 0 | 0 | 0 | 0 |
| BRSSN2015238 | 7.384 | 0 | 0 | 0 | 0 | 0 | 0 |
| BRSSN2015497 | 0 | 0 | 0 | 0 | 0 | 0 | 65.450 |
| BRSSN2015982 | 0 | 0 | 0 | 0 | 0 | 0 | 10.620 |
| BRSTN1000083 | 1.377 | 0 | 0 | 0 | 0 | 0 | 0.835 |
| BRSTN2000367 | 0 | 0 | 0 | 0 | 0 | 0 | 15.347 |
| BRSTN2011961 | 0 | 6.433 | 4.075 | 0 | 6.140 | 1.981 | 0.189 |
| BRSTN2012069 | 0.673 | 2.731 | 0.067 | 7.585 | 1.805 | 2.071 | 0.563 |
| BRSTN2012174 | 0 | 4.492 | 0 | 0 | 8.576 | 2.768 | 0.793 |
| BRSTN2013502 | 0 | 0 | 0 | 0 | 0 | 0 | 1.589 |
| BRSTN2015889 | 0 | 0 | 0 | 0 | 0 | 0 | 2.532 |
| BRSTN2016470 | 1.416 | 0 | 0 | 9.238 | 9.281 | 0 | 2.146 |
| BRSTN2016678 | 0 | 0 | 0 | 0 | 0 | 0 | 5.998 |
| BRSTN2017104 | 0 | 0 | 0 | 0 | 0 | 0 | 1.275 |
| BRTHA2006975 | 0 | 50.497 | 0 | 0 | 0 | 0 | 0 |
| BRTHA2008335 | 0 | 0 | 57.267 | 0 | 0 | 0 | 0 |
| BRTHA2012714 | 0 | 27.840 | 0 | 0 | 0 | 0 | 0 |
| BRTHA2013054 | 0 | 0 | 0 | 0 | 0 | 0 | 31.413 |
| BRTHA2016577 | 0 | 0 | 0 | 0 | 0 | 0 | 3.085 |
| BRTHA2016765 | 0 | 0 | 0 | 0 | 0 | 0 | 7.692 |
| BRTHA2018443 | 0 | 0 | 0 | 0 | 0 | 0 | 28.890 |
| BRTHA2035743 | 14.636 | 0 | 0 | 0 | 0 | 0 | 4.436 |
| BRTHA3000456 | 0 | 0 | 0 | 0 | 0 | 0 | 37.085 |
| BRTHA3003074 | 0 | 0 | 0 | 0 | 0 | 0 | 7.360 |
| BRTHA3003490 | 3.501 | 0 | 0 | 0 | 0 | 0 | 1.061 |
| BRTHA3003736 | 0 | 0 | 0 | 0 | 0 | 0 | 1.406 |
| BRTHA3004307 | 0 | 0 | 0 | 0 | 0 | 0 | 1.969 |
| BRTHA3007472 | 0 | 0 | 0 | 0 | 0 | 0 | 7.652 |
| BRTHA3008778 | 0 | 0 | 0 | 0 | 0 | 0 | 16.240 |
| BRTHA3009090 | 25.386 | 0 | 0 | 0 | 0 | 0 | 0 |
| BRTHA3011194 | 0 | 0 | 0 | 0 | 0 | 0 | 2.310 |
| BRTHA3011361 | 0 | 0 | 0 | 0 | 0 | 0 | 37.085 |
| BRTHA3020314 | 0 | 0 | 0 | 0 | 0 | 0 | 13.312 |
| BRTHA3026507 | 0 | 0 | 0 | 0 | 0 | 0 | 9.136 |
| CD34C3000252 | 0 | 0 | 0 | 0 | 0 | 0 | 1.073 |
| CD34C3000424 | 0 | 0 | 0 | 0 | 0 | 0 | 1.497 |
| CHONS2002829 | 0 | 0 | 0 | 0 | 0 | 0 | 5.340 |
| COLON2001721 | 0 | 0 | 0 | 0 | 0 | 0 | 3.616 |
| CTONG1000052 | 1.303 | 0 | 0 | 12.751 | 0 | 0 | 1.580 |
| CTONG1000087 | 0 | 0 | 0 | 0 | 0 | 0 | 9.570 |
| CTONG1000093 | 0 | 0 | 0 | 0 | 0 | 0 | 25.979 |
| CTONG1000218 | 0 | 0 | 3.387 | 0 | 0 | 3.294 | 0.630 |
| CTONG1000241 | 0 | 0 | 0 | 0 | 0 | 0 | 2.773 |
| CTONG2000063 | 4.617 | 0 | 0 | 0 | 0 | 0 | 8.396 |
| CTONG2000201 | 0 | 0 | 13.636 | 0 | 0 | 0 | 0 |
| CTONG2000452 | 0 | 0 | 0 | 0 | 0 | 19.927 | 1.904 |
| CTONG2000469 | 0 | 0 | 0 | 0 | 0 | 0 | 3.818 |
| CTONG2000508 | 0 | 0 | 0 | 0 | 0 | 0 | 1.032 |
| CTONG2001366 | 0 | 0 | 54.528 | 0 | 0 | 0 | 10.134 |
| CTONG2001748 | 0 | 0 | 14.705 | 0 | 0 | 0 | 0 |
| CTONG2001800 | 0 | 0 | 0 | 0 | 0 | 0 | 18.484 |
| CTONG2001858 | 0 | 0 | 0 | 0 | 0 | 50.495 | 0 |
| CTONG2002073 | 3.159 | 0 | 0 | 0 | 0 | 0 | 0.957 |
| CTONG2002095 | 0 | 0 | 0 | 0 | 0 | 0 | 4.578 |
| CTONG2002143 | 0 | 0 | 0 | 0 | 0 | 0 | 25.979 |
| CTONG2002270 | 0 | 0 | 0 | 0 | 0 | 0 | 12.829 |
| CTONG2002744 | 1.270 | 0 | 0 | 0 | 0 | 0 | 0.770 |
| CTONG2002766 | 0 | 0 | 0 | 0 | 0 | 0 | 23.170 |
| CTONG2002803 | 0 | 0 | 0 | 0 | 0 | 0 | 1.925 |
| CTONG2002832 | 24.179 | 0 | 0 | 0 | 0 | 0 | 14.656 |
| CTONG2002841 | 0 | 0 | 0 | 0 | 0 | 0 | 6.924 |
| CTONG2003189 | 0 | 0 | 0 | 0 | 0 | 0 | 25.979 |
| CTONG2003293 | 19.937 | 0 | 0 | 0 | 0 | 0 | 6.042 |
| CTONG2003298 | 1.565 | 16.118 | 0 | 0 | 0 | 0 | 0 |
| CTONG2003361 | 0 | 0 | 0 | 0 | 0 | 0 | 15.360 |
| CTONG2003517 | 24.179 | 0 | 0 | 0 | 0 | 0 | 0 |
| CTONG2003524 | 0 | 0 | 0 | 0 | 0 | 11.025 | 0 |
| CTONG2003945 | 0 | 0 | 0 | 0 | 0 | 73.112 | 6.985 |
| CTONG2004487 | 0 | 0 | 0 | 0 | 0 | 0 | 7.420 |
| CTONG2005278 | 0 | 0 | 20.273 | 0 | 0 | 0 | 0 |
| CTONG2005553 | 1.545 | 0 | 0 | 0 | 0 | 0 | 0.468 |
| CTONG2005615 | 0 | 0 | 0 | 0 | 0 | 0 | 9.325 |
| CTONG2005913 | 0 | 0 | 31.391 | 0 | 0 | 0 | 2.917 |
| CTONG2006004 | 0 | 0 | 0 | 0 | 0 | 0 | 2.070 |
| CTONG2006235 | 0 | 0 | 0 | 0 | 0 | 0 | 1.071 |
| CTONG2006377 | 0 | 0 | 0 | 0 | 0 | 0 | 23.023 |
| CTONG2006412 | 0 | 0 | 0 | 0 | 0 | 0 | 4.058 |
| CTONG2006562 | 0 | 0 | 0 | 0 | 0 | 0 | 0.915 |
| CTONG2006798 | 0 | 0 | 0 | 0 | 0 | 0 | 6.308 |
| CTONG2006932 | 2.373 | 0 | 0 | 0 | 0 | 0 | 0 |
| CTONG2007007 | 0 | 0 | 0 | 0 | 0 | 6.369 | 1.217 |
| CTONG2007072 | 4.640 | 0 | 0 | 0 | 0 | 0 | 0 |
| CTONG2007078 | 2.756 | 0 | 26.969 | 0 | 0 | 0 | 0 |
| CTONG2007104 | 0 | 0 | 0 | 0 | 0 | 0 | 16.263 |
| CTONG2007399 | 0 | 0 | 0 | 0 | 0 | 0 | 53.310 |
| CTONG2007474 | 0 | 0 | 0 | 0 | 0 | 72.676 | 0 |
| CTONG2007500 | 3.244 | 0 | 0 | 0 | 0 | 5.145 | 0.492 |
| CTONG2007586 | 12.963 | 0 | 0 | 0 | 0 | 0 | 0 |
| CTONG2007613 | 18.879 | 0 | 0 | 0 | 0 | 0 | 5.722 |
| CTONG2007776 | 0 | 0 | 0 | 0 | 0 | 0 | 19.462 |
| CTONG2008398 | 0 | 0 | 0 | 0 | 0 | 5.231 | 1.000 |
| CTONG2008405 | 0 | 0 | 0 | 0 | 0 | 0 | 14.385 |
| CTONG2008466 | 0 | 0 | 54.999 | 0 | 0 | 0 | 0 |
| CTONG2008506 | 0 | 0 | 0 | 0 | 0 | 0 | 0.615 |
| CTONG2008518 | 0 | 0 | 0 | 0 | 0 | 0 | 5.035 |
| CTONG2009529 | 0 | 0 | 0 | 0 | 0 | 43.261 | 0 |
| CTONG2009963 | 0 | 0 | 0 | 0 | 0 | 0 | 27.025 |
| CTONG2010652 | 0 | 0 | 0 | 0 | 0 | 0 | 21.013 |
| CTONG2010821 | 4.624 | 0 | 0 | 0 | 0 | 0 | 0.467 |
| CTONG2011801 | 0 | 0 | 0 | 0 | 0 | 20.259 | 1.936 |
| CTONG2012001 | 0 | 0 | 0 | 0 | 0 | 0 | 4.674 |
| CTONG2012050 | 0 | 0 | 0 | 0 | 0 | 0 | 13.931 |
| CTONG2012101 | 34.050 | 0 | 0 | 0 | 0 | 0 | 20.639 |
| CTONG2012452 | 0.768 | 0 | 2.505 | 0 | 0 | 0 | 0.931 |
| CTONG2012607 | 0 | 0 | 0 | 0 | 0 | 0 | 2.261 |
| CTONG2012745 | 0 | 0 | 0 | 0 | 0 | 0 | 3.626 |
| CTONG2012847 | 3.714 | 0 | 0 | 0 | 0 | 0 | 2.251 |
| CTONG2013178 | 0 | 0 | 0 | 0 | 0 | 64.748 | 0 |
| CTONG2013222 | 0 | 0 | 0 | 0 | 79.674 | 0 | 0 |
| CTONG2013348 | 0 | 0 | 0 | 0 | 0 | 0 | 3.596 |
| CTONG2013934 | 0 | 0 | 0 | 0 | 0 | 0 | 4.534 |
| CTONG2014369 | 0 | 0 | 0 | 0 | 0 | 28.473 | 5.441 |
| CTONG2014898 | 12.440 | 0 | 0 | 0 | 0 | 0 | 3.770 |
| CTONG2015091 | 0 | 0 | 0 | 0 | 0 | 0 | 0.531 |
| CTONG2015330 | 0 | 0 | 0 | 0 | 0 | 0 | 18.590 |
| CTONG2015358 | 0 | 0 | 0 | 0 | 0 | 0 | 25.979 |
| CTONG2016185 | 0 | 0 | 0 | 0 | 0 | 17.787 | 0 |
| CTONG2016408 | 7.985 | 0 | 0 | 0 | 0 | 0 | 4.840 |
| CTONG2016575 | 1.072 | 0 | 0 | 0 | 0 | 0 | 2.275 |
| CTONG2016869 | 0 | 0 | 0 | 0 | 0 | 0 | 1.727 |
| CTONG2016931 | 0 | 0 | 0 | 0 | 0 | 0 | 3.990 |
| CTONG2016942 | 0 | 0 | 0 | 0 | 0 | 78.602 | 0 |
| CTONG2017094 | 0 | 0 | 0 | 0 | 0 | 0 | 5.846 |
| CTONG2017429 | 0 | 0 | 0 | 0 | 0 | 0 | 9.435 |
| CTONG2017500 | 0 | 0 | 0 | 0 | 0 | 0 | 10.390 |
| CTONG2017798 | 0 | 0 | 0 | 0 | 0 | 0 | 13.027 |
| CTONG2017998 | 0 | 0 | 0 | 0 | 0 | 0 | 3.275 |
| CTONG2018383 | 9.750 | 0 | 0 | 0 | 0 | 0 | 0 |
| CTONG2018632 | 0 | 0 | 0 | 0 | 0 | 0 | 5.228 |
| CTONG2018637 | 0 | 0 | 0 | 0 | 0 | 0 | 4.288 |
| CTONG2018652 | 0 | 0 | 0 | 0 | 0 | 0 | 25.979 |
| CTONG2018739 | 0 | 0 | 0 | 0 | 0 | 0 | 5.066 |
| CTONG2018808 | 16.987 | 0 | 0 | 0 | 0 | 0 | 10.297 |
| CTONG2018843 | 23.945 | 0 | 0 | 0 | 0 | 0 | 7.257 |
| CTONG2018898 | 2.077 | 0 | 0 | 0 | 0 | 0 | 0 |
| CTONG2019400 | 0 | 0 | 0 | 0 | 0 | 0 | 38.711 |
| CTONG2019788 | 0 | 0 | 0 | 0 | 0 | 0 | 1.999 |
| CTONG2020484 | 0 | 0 | 4.135 | 0 | 0 | 0 | 0 |
| CTONG2020522 | 0 | 0 | 0 | 0 | 0 | 0 | 14.980 |
| CTONG2020560 | 0 | 0 | 0 | 0 | 0 | 10.264 | 4.903 |
| CTONG2020582 | 0 | 0 | 0 | 0 | 0 | 0 | 12.542 |
| CTONG2020638 | 26.704 | 0 | 0 | 0 | 0 | 0 | 0 |
| CTONG2020974 | 0 | 0 | 0 | 0 | 0 | 0 | 25.979 |
| CTONG2021289 | 0 | 0 | 0 | 0 | 0 | 0 | 6.726 |
| CTONG2023449 | 0 | 0 | 0 | 0 | 0 | 78.602 | 0 |
| CTONG2027263 | 0 | 0 | 0 | 0 | 0 | 0 | 21.530 |
| CTONG2028124 | 7.588 | 0 | 0 | 0 | 0 | 0 | 1.314 |
| CTONG2028309 | 15.831 | 0 | 0 | 0 | 0 | 0 | 14.394 |
| CTONG2028758 | 0 | 0 | 0 | 0 | 0 | 0 | 10.513 |
| CTONG3001123 | 0 | 0 | 0 | 0 | 0 | 0 | 15.389 |
| CTONG3002518 | 0 | 0 | 0 | 0 | 0 | 57.732 | 0 |
| CTONG3002835 | 2.370 | 0 | 0 | 0 | 0 | 0 | 5.745 |
| CTONG3006067 | 0 | 0 | 0 | 0 | 0 | 0 | 25.979 |
| CTONG3008894 | 1.568 | 16.146 | 0 | 0 | 0 | 0 | 2.376 |
| CTONG3009028 | 0 | 0 | 0 | 0 | 0 | 0 | 2.104 |
| CTONG3009239 | 0 | 32.069 | 0 | 0 | 0 | 0 | 0 |
| D30ST2001670 | 0 | 0 | 0 | 0 | 0 | 0 | 31.707 |
| D30ST2002068 | 0.463 | 0 | 0 | 0 | 0 | 0 | 0 |
| D30ST2003024 | 0 | 0 | 0 | 0 | 0 | 0 | 5.302 |
| D30ST3000169 | 0 | 0 | 0 | 0 | 0 | 6.696 | 0 |
| D90ST2000031 | 0 | 0 | 0 | 0 | 0 | 0 | 3.088 |
| D90ST2003762 | 0 | 48.412 | 0 | 0 | 0 | 0 | 0 |
| DFNES1000150 | 14.452 | 0 | 0 | 0 | 0 | 0 | 0 |
| DFNES2000011 | 0 | 0 | 0 | 0 | 0 | 0 | 0.357 |
| DFNES2000153 | 0 | 0 | 0 | 0 | 0 | 21.906 | 0 |
| DFNES2000268 | 0 | 24.526 | 0 | 0 | 0 | 0 | 0 |
| DFNES2000335 | 0 | 0 | 0 | 0 | 0 | 0 | 9.291 |
| DFNES2000443 | 0 | 15.077 | 0 | 0 | 0 | 4.644 | 0 |
| DFNES2000526 | 5.960 | 0 | 0 | 0 | 0 | 0 | 0 |
| DFNES2002966 | 0 | 0 | 0 | 0 | 0 | 0 | 9.984 |
| DFNES2003192 | 0 | 66.531 | 0 | 0 | 0 | 0 | 0 |
| DFNES2004346 | 0 | 0 | 0 | 0 | 0 | 0 | 2.046 |
| DFNES2004371 | 0 | 35.042 | 0 | 0 | 0 | 0 | 2.063 |
| DFNES2007113 | 0 | 0 | 0 | 0 | 0 | 45.721 | 0 |
| DFNES2007634 | 5.751 | 0 | 0 | 0 | 0 | 0 | 3.486 |
| DFNES2008088 | 5.670 | 0 | 0 | 0 | 0 | 0 | 0 |
| DFNES2011380 | 0 | 0 | 0 | 0 | 0 | 0 | 7.641 |
| DFNES2011499 | 0 | 0 | 0 | 0 | 0 | 0 | 12.166 |
| FCBBF1000024 | 0 | 0 | 0 | 0 | 0 | 0 | 1.435 |
| FCBBF1000027 | 0.790 | 0 | 0 | 0 | 0 | 2.506 | 0.239 |
| FCBBF1000061 | 0 | 0 | 0 | 0 | 0 | 0 | 1.537 |
| FCBBF1000063 | 0 | 0 | 0 | 0 | 0 | 0 | 3.910 |
| FCBBF1000077 | 0 | 0 | 5.697 | 0 | 0 | 0 | 0 |
| FCBBF1000139 | 0 | 0 | 0 | 0 | 0 | 0 | 1.143 |
| FCBBF1000197 | 0 | 0 | 0 | 0 | 0 | 0 | 2.793 |
| FCBBF1000294 | 0 | 0 | 7.574 | 0 | 0 | 0 | 2.111 |
| FCBBF1000374 | 0 | 0 | 0 | 0 | 0 | 0 | 26.135 |
| FCBBF1000425 | 11.260 | 0 | 0 | 0 | 0 | 0 | 10.238 |
| FCBBF1000563 | 0 | 0 | 0 | 0 | 0 | 0 | 0.982 |
| FCBBF1000574 | 0 | 0 | 0 | 0 | 0 | 0 | 1.357 |
| FCBBF1000760 | 0 | 0 | 0 | 0 | 0 | 0 | 3.425 |
| FCBBF2000087 | 6.222 | 0 | 0 | 0 | 0 | 0 | 0 |
| FCBBF2000195 | 0 | 92.321 | 0 | 0 | 0 | 0 | 0 |
| FCBBF2000199 | 0 | 0 | 0 | 0 | 0 | 0 | 1.829 |
| FCBBF2000576 | 0 | 0 | 79.200 | 0 | 0 | 0 | 0 |
| FCBBF2000591 | 0 | 0 | 0 | 0 | 0 | 0 | 0.375 |
| FCBBF2000885 | 3.311 | 0 | 0 | 0 | 0 | 0 | 2.007 |
| FCBBF2002111 | 0 | 0 | 0 | 0 | 0 | 78.739 | 0 |
| FCBBF2002349 | 0 | 0 | 0 | 0 | 0 | 0 | 7.742 |
| FCBBF2002801 | 0 | 0 | 0 | 0 | 0 | 17.757 | 0 |
| FCBBF2003102 | 0 | 0 | 0 | 0 | 0 | 0 | 13.089 |
| FCBBF2005658 | 0 | 0 | 0 | 0 | 0 | 0 | 5.088 |
| FCBBF3000110 | 13.929 | 0 | 0 | 0 | 0 | 0 | 0 |
| FCBBF3000228 | 38.659 | 0 | 0 | 0 | 0 | 0 | 5.858 |
| FCBBF3000388 | 0 | 0 | 0 | 0 | 0 | 0 | 1.615 |
| FCBBF3000434 | 0 | 0 | 0 | 0 | 0 | 0 | 2.420 |
| FCBBF3000536 | 0 | 0 | 0 | 0 | 0 | 0 | 1.742 |
| FCBBF3001081 | 11.326 | 0 | 18.471 | 0 | 0 | 0 | 0 |
| FCBBF3001281 | 0 | 0 | 0 | 0 | 0 | 0 | 13.310 |
| FCBBF3001377 | 0 | 0 | 0 | 0 | 0 | 0 | 20.193 |
| FCBBF3001470 | 0 | 0 | 0 | 0 | 0 | 0 | 0.887 |
| FCBBF3001632 | 3.330 | 0 | 0 | 0 | 0 | 0 | 1.009 |
| FCBBF3001818 | 0 | 0 | 0 | 0 | 0 | 0 | 5.954 |
| FCBBF3001977 | 0 | 0 | 0 | 0 | 0 | 0 | 4.869 |
| FCBBF3003800 | 0.850 | 0 | 0 | 0 | 0 | 0 | 0.257 |
| FCBBF3004261 | 0 | 0 | 0 | 0 | 0 | 0 | 11.826 |
| FCBBF3004473 | 0 | 0 | 8.082 | 0 | 0 | 0 | 0.751 |
| FCBBF3005330 | 1.494 | 0 | 4.873 | 0 | 0 | 0 | 13.585 |
| FCBBF3005497 | 0 | 0 | 0 | 0 | 0 | 0 | 10.792 |
| FCBBF3006821 | 0 | 0 | 0 | 0 | 0 | 0 | 22.420 |
| FCBBF3007597 | 53.863 | 0 | 0 | 0 | 0 | 0 | 0 |
| FCBBF3008159 | 0 | 0 | 0 | 0 | 0 | 0 | 40.612 |
| FCBBF3008556 | 0 | 0 | 0 | 0 | 0 | 0 | 26.135 |
| FCBBF3009541 | 1.778 | 0 | 0 | 0 | 0 | 0 | 1.347 |
| FCBBF3011485 | 0 | 0 | 0 | 0 | 0 | 0 | 13.111 |
| FCBBF3012288 | 0 | 0 | 0 | 0 | 0 | 0 | 26.135 |
| FCBBF3012347 | 0 | 48.750 | 0 | 0 | 0 | 15.017 | 1.435 |
| FCBBF3013266 | 0 | 0 | 0 | 0 | 0 | 0 | 13.819 |
| FCBBF3013506 | 0 | 0 | 0 | 0 | 0 | 0 | 15.524 |
| FCBBF3014770 | 0 | 0 | 0 | 0 | 0 | 0 | 6.238 |
| FCBBF3015727 | 0 | 0 | 0 | 0 | 0 | 0 | 26.135 |
| FCBBF3016928 | 0 | 19.462 | 0 | 0 | 0 | 0 | 0.573 |
| FCBBF3017396 | 0 | 0 | 0 | 0 | 0 | 0 | 5.338 |
| FCBBF3017531 | 0 | 0 | 0 | 0 | 0 | 10.970 | 0 |
| FCBBF3017918 | 0 | 0 | 0 | 0 | 0 | 0 | 8.560 |
| FCBBF3018796 | 0 | 0 | 21.839 | 0 | 0 | 0 | 2.029 |
| FCBBF3019455 | 0 | 0 | 0 | 0 | 0 | 0 | 10.647 |
| FCBBF3019839 | 0 | 0 | 0 | 0 | 0 | 0 | 2.448 |
| FCBBF3021807 | 9.352 | 0 | 30.500 | 0 | 0 | 0 | 0 |
| FCBBF3021940 | 53.863 | 0 | 0 | 0 | 0 | 0 | 0 |
| FCBBF3023667 | 0 | 0 | 0 | 0 | 0 | 0.909 | 0.434 |
| FCBBF3024002 | 12.241 | 0 | 0 | 0 | 0 | 0 | 3.710 |
| FCBBF3025730 | 0 | 0 | 0 | 0 | 0 | 0 | 38.827 |
| FCBBF3025737 | 0 | 0 | 0 | 0 | 0 | 0 | 4.249 |
| FCBBF3026651 | 0 | 0 | 0 | 0 | 0 | 0 | 19.080 |
| FCBBF3028202 | 0 | 0 | 0 | 0 | 0 | 0 | 15.020 |
| FCBBF3028593 | 0 | 0 | 0 | 0 | 0 | 0 | 2.639 |
| FCBBF4000446 | 0 | 0 | 5.284 | 0 | 0 | 0 | 0 |
| FCBBF4000500 | 0 | 0 | 0 | 0 | 0 | 0 | 5.729 |
| FCBBF4000548 | 31.525 | 0 | 0 | 0 | 0 | 0 | 0 |
| FCBBF5000261 | 0 | 0 | 0 | 0 | 0 | 0 | 6.788 |
| FEBRA1000022 | 11.352 | 0 | 0 | 0 | 0 | 0 | 0 |
| FEBRA1000057 | 0 | 0 | 0 | 0 | 0 | 0 | 28.370 |
| FEBRA1000088 | 0 | 0 | 0 | 0 | 0 | 29.198 | 0 |
| FEBRA2000105 | 0 | 0 | 0 | 0 | 0 | 0 | 0.469 |
| FEBRA2000210 | 0 | 0 | 0 | 14.918 | 0 | 0 | 0 |
| FEBRA2000282 | 0 | 0 | 0 | 0 | 0 | 0 | 20.775 |
| FEBRA2000311 | 0 | 0 | 0 | 0 | 0 | 68.498 | 6.544 |
| FEBRA2000438 | 0 | 0 | 0 | 0 | 0 | 0 | 5.775 |
| FEBRA2000452 | 0 | 0 | 0 | 0 | 0 | 6.862 | 0.656 |
| FEBRA2000510 | 0 | 0 | 0 | 0 | 0 | 25.245 | 0 |
| FEBRA2000532 | 0 | 0 | 0 | 0 | 0 | 21.879 | 0 |
| FEBRA2000733 | 0.727 | 7.486 | 7.113 | 0 | 0 | 1.153 | 0.110 |
| FEBRA2000740 | 0 | 0 | 0 | 0 | 0 | 7.738 | 1.479 |
| FEBRA2000762 | 0 | 0 | 0 | 0 | 0 | 0 | 4.066 |
| FEBRA2000856 | 0 | 0 | 0 | 0 | 0 | 0 | 41.502 |
| FEBRA2000881 | 0 | 0 | 0 | 0 | 0 | 0 | 8.276 |
| FEBRA2000972 | 32.671 | 0 | 0 | 0 | 0 | 0 | 0 |
| FEBRA2001133 | 0 | 0 | 0 | 0 | 0 | 0 | 20.775 |
| FEBRA2001334 | 12.403 | 0 | 0 | 0 | 0 | 0 | 0 |
| FEBRA2001492 | 0 | 0 | 0 | 0 | 0 | 0 | 4.231 |
| FEBRA2001523 | 0 | 0 | 0 | 0 | 0 | 0 | 5.558 |
| FEBRA2001571 | 4.523 | 0 | 0 | 0 | 0 | 0 | 1.371 |
| FEBRA2001752 | 0 | 0 | 0 | 0 | 0 | 0 | 10.296 |
| FEBRA2001828 | 0 | 0 | 0 | 0 | 0 | 0 | 7.113 |
| FEBRA2002429 | 0 | 86.973 | 0 | 0 | 0 | 0 | 0 |
| FEBRA2002527 | 0 | 0 | 0 | 0 | 0 | 0 | 5.616 |
| FEBRA2002707 | 0 | 0 | 0 | 0 | 0 | 0 | 2.355 |
| FEBRA2002933 | 0 | 0 | 2.603 | 0 | 0 | 0 | 0.242 |
| FEBRA2003100 | 1.420 | 0 | 0 | 0 | 0 | 0 | 0.431 |
| FEBRA2003833 | 0 | 0 | 0 | 0 | 0 | 0 | 2.261 |
| FEBRA2003926 | 0 | 15.505 | 4.911 | 0 | 0 | 0 | 0 |
| FEBRA2004023 | 0 | 0 | 0 | 0 | 0 | 0 | 20.775 |
| FEBRA2004191 | 0 | 0 | 0 | 0 | 0 | 0 | 25.927 |
| FEBRA2004224 | 2.497 | 0 | 0 | 0 | 0 | 0 | 0 |
| FEBRA2004293 | 0 | 0 | 0 | 0 | 0 | 0 | 1.738 |
| FEBRA2004412 | 0 | 0 | 0 | 0 | 0 | 0 | 1.919 |
| FEBRA2005416 | 0 | 0 | 0 | 0 | 0 | 0 | 1.404 |
| FEBRA2005734 | 0 | 0 | 5.940 | 0 | 0 | 0 | 0 |
| FEBRA2005752 | 0 | 0 | 0 | 0 | 0 | 0 | 2.721 |
| FEBRA2005995 | 3.024 | 0 | 0 | 0 | 0 | 9.591 | 10.080 |
| FEBRA2006092 | 0 | 0 | 0 | 0 | 0 | 0 | 2.439 |
| FEBRA2006112 | 0 | 0 | 0 | 0 | 0 | 0 | 21.573 |
| FEBRA2006270 | 2.521 | 0 | 0 | 0 | 0 | 0 | 1.528 |
| FEBRA2006368 | 0 | 0 | 0 | 0 | 0 | 0 | 4.062 |
| FEBRA2006372 | 2.897 | 0 | 0 | 0 | 0 | 0 | 0.878 |
| FEBRA2006401 | 0 | 0 | 0 | 0 | 0 | 1.717 | 0 |
| FEBRA2006664 | 0 | 0 | 0 | 0 | 0 | 0 | 20.775 |
| FEBRA2007200 | 0 | 0 | 0 | 0 | 0 | 0 | 20.775 |
| FEBRA2007212 | 0 | 0 | 0 | 0 | 0 | 0 | 10.264 |
| FEBRA2007620 | 0 | 0 | 0 | 0 | 0 | 0 | 20.775 |
| FEBRA2007622 | 2.225 | 0 | 0 | 0 | 0 | 7.059 | 2.023 |
| FEBRA2007708 | 0 | 0 | 0 | 0 | 0 | 0 | 72.628 |
| FEBRA2007801 | 0 | 0 | 0 | 0 | 0 | 0 | 3.227 |
| FEBRA2007900 | 0 | 0 | 0 | 0 | 0 | 0 | 5.067 |
| FEBRA2007901 | 0 | 0 | 0 | 0 | 0 | 0 | 2.423 |
| FEBRA2008036 | 0 | 0 | 0 | 0 | 0 | 0 | 5.602 |
| FEBRA2008081 | 0 | 0 | 9.963 | 0 | 0 | 0 | 0.926 |
| FEBRA2008087 | 1.160 | 0 | 2.522 | 7.566 | 0 | 0 | 0.352 |
| FEBRA2008583 | 0 | 0 | 0 | 0 | 0 | 0.904 | 0.086 |
| FEBRA2008755 | 3.543 | 0 | 0 | 0 | 0 | 0 | 0 |
| FEBRA2008836 | 0 | 0 | 0 | 0 | 0 | 0 | 20.775 |
| FEBRA2009328 | 0 | 0 | 0 | 0 | 0 | 67.807 | 0 |
| FEBRA2009419 | 5.700 | 0 | 0 | 0 | 0 | 0 | 1.727 |
| FEBRA2009514 | 0 | 0 | 0 | 0 | 0 | 45.326 | 2.165 |
| FEBRA2009986 | 0 | 0 | 0 | 0 | 0 | 0 | 4.202 |
| FEBRA2014297 | 0 | 0 | 0 | 0 | 0 | 0 | 2.794 |
| FEBRA2014417 | 0 | 11.993 | 3.798 | 0 | 0 | 0 | 0.353 |
| FEBRA2015085 | 0 | 0 | 0 | 0 | 0 | 0 | 0.997 |
| FEBRA2016572 | 0 | 0 | 0 | 0 | 0 | 0 | 12.844 |
| FEBRA2018476 | 2.357 | 0 | 0 | 23.059 | 0 | 0 | 0.714 |
| FEBRA2018746 | 0 | 0 | 0 | 0 | 0 | 51.169 | 0 |
| FEBRA2019663 | 0 | 0 | 61.269 | 0 | 0 | 0 | 0 |
| FEBRA2019711 | 0 | 0 | 14.875 | 0 | 0 | 0 | 1.382 |
| FEBRA2021339 | 0 | 0 | 0 | 0 | 0 | 0 | 20.775 |
| FEBRA2021966 | 9.304 | 0 | 0 | 0 | 0 | 0 | 0 |
| FEBRA2022013 | 0 | 0 | 66.137 | 0 | 0 | 0 | 0 |
| FEBRA2022055 | 1.148 | 0 | 0 | 0 | 0 | 0 | 0.348 |
| FEBRA2022504 | 0 | 0 | 0 | 0 | 0 | 0 | 3.221 |
| FEBRA2024150 | 0 | 0 | 0 | 0 | 0 | 0 | 44.030 |
| FEBRA2024987 | 0 | 0 | 0 | 0 | 0 | 0 | 6.430 |
| FEBRA2025427 | 0 | 0 | 0 | 0 | 0 | 16.960 | 0 |
| FEBRA2025838 | 0 | 0 | 0 | 17.336 | 0 | 0 | 0.537 |
| FEBRA2026984 | 0 | 0 | 0 | 0 | 0 | 0 | 22.925 |
| FEBRA2028516 | 0 | 0 | 0 | 0 | 0 | 0 | 4.486 |
| FELIV1000153 | 0 | 0 | 1.160 | 0 | 0 | 1.128 | 0.323 |
| HCASM1000115 | 2.236 | 0 | 0 | 0 | 0 | 0 | 0 |
| HCASM2000016 | 0 | 0 | 0 | 0 | 0 | 0 | 0.488 |
| HCASM2000138 | 0 | 0 | 0 | 0 | 0 | 0 | 11.043 |
| HCASM2000363 | 0 | 15.104 | 0 | 14.351 | 0 | 0 | 0 |
| HCASM2003018 | 0 | 0 | 0 | 0 | 0 | 0 | 23.077 |
| HCHON1000030 | 0.782 | 0 | 3.828 | 0 | 3.845 | 0 | 0.356 |
| HCHON1000131 | 0 | 0 | 0 | 0 | 0 | 0 | 5.364 |
| HCHON1000166 | 0 | 0 | 0 | 0 | 0 | 0 | 1.966 |
| HCHON2000056 | 0 | 0 | 1.995 | 0 | 6.013 | 5.822 | 0.371 |
| HCHON2000087 | 1.334 | 0 | 0 | 0 | 0 | 0 | 0 |
| HCHON2000160 | 2.171 | 0 | 0 | 0 | 0 | 0 | 1.316 |
| HCHON2000226 | 0 | 0 | 11.347 | 0 | 17.098 | 0 | 0.264 |
| HCHON2000244 | 0 | 0 | 4.627 | 0 | 0 | 4.501 | 0.430 |
| HCHON2000265 | 0 | 0 | 0 | 0 | 0 | 0 | 1.323 |
| HCHON2000323 | 0 | 0 | 0 | 0 | 0 | 0 | 1.211 |
| HCHON2000373 | 0 | 0 | 0 | 0 | 0 | 6.934 | 0.663 |
| HCHON2000475 | 2.871 | 0 | 0 | 0 | 0 | 9.107 | 0 |
| HCHON2000508 | 4.027 | 0 | 0 | 0 | 0 | 0 | 0 |
| HCHON2000626 | 2.535 | 0 | 0 | 0 | 0 | 0 | 0.768 |
| HCHON2000663 | 18.399 | 0 | 0 | 0 | 0 | 0 | 0 |
| HCHON2000698 | 6.182 | 0 | 0 | 0 | 0 | 0 | 5.621 |
| HCHON2000738 | 0 | 0 | 0 | 0 | 0 | 0 | 5.839 |
| HCHON2000815 | 0 | 0 | 0 | 0 | 0 | 0 | 1.826 |
| HCHON2000826 | 4.631 | 15.897 | 0 | 0 | 0 | 0 | 0.468 |
| HCHON2001039 | 4.813 | 0 | 7.849 | 0 | 0 | 0 | 3.647 |
| HCHON2001099 | 0 | 0 | 0 | 0 | 0 | 0 | 1.783 |
| HCHON2001200 | 0.933 | 0 | 0 | 0 | 0 | 0 | 0 |
| HCHON2001217 | 0 | 0 | 0 | 0 | 0 | 0 | 4.763 |
| HCHON2001359 | 1.836 | 0 | 0 | 0 | 0 | 0 | 0.557 |
| HCHON2001407 | 0 | 0 | 0 | 0 | 0 | 0 | 2.065 |
| HCHON2001505 | 9.692 | 0 | 0 | 0 | 0 | 0 | 2.937 |
| HCHON2001519 | 0 | 0 | 0 | 0 | 0 | 0 | 9.363 |
| HCHON2001535 | 0 | 0 | 0 | 0 | 0 | 0 | 6.028 |
| HCHON2001598 | 0 | 0 | 0 | 0 | 0 | 0 | 1.588 |
| HCHON2001665 | 6.657 | 0 | 0 | 0 | 0 | 10.558 | 1.009 |
| HCHON2002496 | 0 | 0 | 0 | 0 | 0 | 0 | 5.077 |
| HCHON2004002 | 8.467 | 0 | 5.523 | 0 | 0 | 0 | 0 |
| HCHON2005921 | 8.945 | 0 | 0 | 0 | 0 | 0 | 2.711 |
| HCHON2006250 | 0 | 0 | 11.007 | 0 | 0 | 0 | 2.046 |
| HCHON2006770 | 0 | 62.687 | 0 | 0 | 0 | 0 | 0 |
| HEART1000010 | 0 | 0 | 24.034 | 0 | 0 | 0 | 0 |
| HEART1000149 | 0 | 0 | 0 | 0 | 0 | 0 | 0.614 |
| HEART1000185 | 0 | 0 | 0 | 0 | 0 | 0 | 0.517 |
| HEART2000444 | 0 | 0 | 0 | 0 | 0 | 0 | 5.505 |
| HEART2000506 | 3.782 | 0 | 0 | 0 | 0 | 0 | 0 |
| HEART2003836 | 0 | 0 | 0 | 0 | 0 | 0 | 1.345 |
| HEART2004764 | 0 | 0 | 0 | 0 | 0 | 0 | 16.386 |
| HEART2004931 | 0 | 0 | 0 | 0 | 0 | 46.525 | 0 |
| HEART2004941 | 0 | 0 | 0 | 0 | 0 | 0 | 3.105 |
| HEART2005310 | 0 | 0 | 0 | 0 | 0 | 0 | 4.557 |
| HEART2006521 | 5.159 | 0 | 0 | 0 | 0 | 0 | 0 |
| HEART2007696 | 0 | 0 | 0 | 0 | 0 | 0 | 3.256 |
| HHDPC1000083 | 2.111 | 0 | 0 | 0 | 0 | 0 | 0.640 |
| HHDPC1000114 | 0 | 0 | 0 | 0 | 0 | 0 | 0.475 |
| HHDPC1000118 | 0 | 0 | 0 | 0 | 0 | 14.339 | 1.370 |
| HHDPC1000163 | 2.712 | 0 | 0 | 0 | 0 | 0 | 0 |
| HHDPC2000070 | 0 | 0 | 0 | 0 | 0 | 0 | 6.291 |
| HHDPC2000102 | 0 | 0 | 0 | 0 | 0 | 32.486 | 3.104 |
| HHDPC2000104 | 3.990 | 0 | 0 | 0 | 0 | 0 | 0 |
| HHDPC2000115 | 5.642 | 0 | 0 | 0 | 0 | 0 | 0 |
| HHDPC2000455 | 3.556 | 0 | 0 | 0 | 0 | 0 | 0 |
| HHDPC2003439 | 0.549 | 0 | 1.789 | 0 | 0 | 0 | 0.665 |
| HHDPC2003472 | 0 | 0 | 0 | 0 | 0 | 0 | 8.495 |
| HHDPC2003983 | 1.201 | 0 | 0 | 5.877 | 0 | 0 | 1.274 |
| HHDPC2005794 | 0 | 0 | 0 | 0 | 0 | 0 | 5.200 |
| HHDPC2007267 | 0 | 0 | 10.303 | 0 | 0 | 0 | 0.319 |
| HHDPC2009178 | 21.893 | 0 | 0 | 0 | 0 | 0 | 6.635 |
| HHDPC2009528 | 0 | 0 | 0 | 0 | 0 | 0 | 8.497 |
| HLUNG1000037 | 0 | 0 | 0 | 0 | 0 | 0 | 5.744 |
| HLUNG1000055 | 0 | 0 | 3.808 | 0 | 0 | 0 | 1.415 |
| HLUNG1000099 | 0 | 0 | 0 | 0 | 0 | 0 | 28.221 |
| HLUNG1000110 | 0 | 0 | 0 | 0 | 0 | 0 | 15.126 |
| HLUNG2000004 | 0 | 0 | 17.793 | 0 | 0 | 0 | 0 |
| HLUNG2000314 | 0 | 0 | 0 | 0 | 0 | 0 | 1.604 |
| HLUNG2000501 | 0 | 0 | 0 | 0 | 0 | 0 | 1.682 |
| HLUNG2000846 | 0 | 0 | 0 | 0 | 0 | 0 | 2.953 |
| HLUNG2000884 | 0 | 0 | 0 | 0 | 0 | 14.645 | 2.798 |
| HLUNG2001146 | 0 | 0 | 0 | 0 | 0 | 0 | 3.427 |
| HLUNG2001518 | 0 | 0 | 0 | 0 | 0 | 0 | 2.144 |
| HLUNG2002648 | 0 | 0 | 0 | 0 | 0 | 0 | 2.363 |
| HLUNG2003003 | 0 | 0 | 0 | 0 | 0 | 0 | 5.224 |
| HLUNG2003042 | 0 | 0 | 16.745 | 0 | 0 | 0 | 0 |
| HLUNG2004159 | 3.910 | 0 | 0 | 0 | 0 | 0 | 3.555 |
| HLUNG2004534 | 0 | 0 | 20.405 | 0 | 0 | 0 | 0 |
| HLUNG2005133 | 0 | 0 | 0 | 0 | 0 | 0 | 13.892 |
| HLUNG2005230 | 0 | 0 | 16.080 | 0 | 0 | 0 | 0 |
| HLUNG2008139 | 0 | 0 | 0 | 0 | 0 | 0 | 14.983 |
| HLUNG2008225 | 0 | 0 | 0 | 0 | 0 | 0 | 10.785 |
| HLUNG2008637 | 0 | 0 | 11.883 | 0 | 0 | 0 | 2.208 |
| HLUNG2008885 | 0 | 0 | 0 | 0 | 0 | 0 | 7.056 |
| HLUNG2009225 | 6.506 | 0 | 0 | 0 | 0 | 0 | 1.972 |
| HLUNG2017262 | 3.420 | 0 | 0 | 0 | 0 | 0 | 14.513 |
| HSYRA1000062 | 0 | 0 | 0 | 0 | 0 | 0 | 0.280 |
| HSYRA1000119 | 1.983 | 0 | 3.234 | 0 | 0 | 0 | 3.306 |
| HSYRA1000137 | 0 | 0 | 0 | 0 | 0 | 0 | 2.436 |
| HSYRA1000148 | 0 | 0 | 0 | 0 | 0 | 0 | 4.311 |
| HSYRA1000152 | 2.573 | 0 | 0 | 0 | 0 | 0 | 2.339 |
| HSYRA1000178 | 0 | 0 | 0 | 0 | 0 | 0 | 4.917 |
| HSYRA2000159 | 0.520 | 0 | 0 | 0 | 0 | 2.475 | 0 |
| HSYRA2000190 | 0 | 0 | 0 | 0 | 0 | 0 | 4.658 |
| HSYRA2000224 | 3.878 | 0 | 0 | 0 | 0 | 0 | 1.175 |
| HSYRA2000232 | 0 | 0 | 0 | 0 | 0 | 0 | 1.450 |
| HSYRA2000255 | 0 | 29.899 | 0 | 0 | 0 | 0 | 0 |
| HSYRA2000507 | 0 | 0 | 0 | 0 | 0 | 0 | 8.042 |
| HSYRA2000640 | 3.675 | 0 | 0 | 0 | 0 | 5.829 | 2.228 |
| HSYRA2000743 | 3.338 | 0 | 0 | 0 | 0 | 0 | 2.529 |
| HSYRA2000787 | 0 | 0 | 0 | 0 | 0 | 0 | 2.033 |
| HSYRA2001103 | 1.829 | 0 | 0 | 0 | 0 | 0 | 0 |
| HSYRA2001138 | 0 | 0 | 0 | 0 | 0 | 0 | 0.500 |
| HSYRA2001225 | 0 | 0 | 0 | 0 | 0 | 0 | 1.933 |
| HSYRA2001396 | 0 | 0 | 0 | 0 | 0 | 10.394 | 0 |
| HSYRA2001420 | 12.017 | 0 | 0 | 0 | 0 | 0 | 0 |
| HSYRA2001476 | 5.206 | 0 | 0 | 0 | 0 | 0 | 0.316 |
| HSYRA2001567 | 4.834 | 0 | 0 | 0 | 0 | 0 | 0 |
| HSYRA2001595 | 0 | 0 | 4.007 | 0 | 0 | 0 | 2.607 |
| HSYRA2001615 | 0 | 0 | 0 | 0 | 0 | 0 | 1.356 |
| HSYRA2001621 | 0 | 0 | 0 | 0 | 0 | 1.255 | 0 |
| HSYRA2001631 | 0 | 0 | 0 | 0 | 0 | 0 | 3.812 |
| HSYRA2001638 | 4.878 | 0 | 0 | 0 | 0 | 0 | 1.478 |
| IMR321000210 | 0 | 0 | 7.130 | 0 | 0 | 0 | 0.663 |
| IMR321000219 | 1.816 | 6.234 | 0 | 0 | 0 | 0 | 0.183 |
| IMR321000242 | 0 | 0 | 0 | 0 | 0 | 0 | 7.352 |
| IMR321000253 | 2.197 | 0 | 0 | 0 | 0 | 0 | 0.666 |
| IMR321000266 | 0 | 0 | 0 | 0 | 0 | 0 | 1.850 |
| IMR322000010 | 0 | 0 | 66.596 | 0 | 0 | 0 | 0 |
| IMR322000144 | 0 | 0 | 0 | 0 | 0 | 0 | 2.132 |
| IMR322000223 | 0 | 0 | 9.579 | 0 | 0 | 9.317 | 1.780 |
| IMR322000243 | 1.850 | 0 | 0 | 0 | 0 | 0 | 1.682 |
| IMR322000555 | 0 | 0 | 0 | 0 | 0 | 0 | 3.900 |
| IMR322000730 | 0.296 | 3.044 | 0 | 11.570 | 0 | 0.938 | 1.165 |
| IMR322000742 | 0 | 0 | 0 | 0 | 0 | 0 | 1.082 |
| IMR322001332 | 0 | 0 | 0 | 0 | 0 | 18.216 | 6.962 |
| IMR322001380 | 0 | 0 | 0 | 0 | 0 | 0 | 1.296 |
| IMR322001435 | 2.026 | 0 | 0 | 0 | 0 | 0 | 1.228 |
| IMR322001491 | 0 | 0 | 0 | 0 | 0 | 65.975 | 0 |
| IMR322001584 | 0 | 0 | 0 | 0 | 0 | 0 | 0.816 |
| IMR322001600 | 0 | 0 | 1.133 | 0 | 0 | 3.305 | 0 |
| IMR322001710 | 8.366 | 0 | 0 | 0 | 0 | 0 | 6.339 |
| IMR322002993 | 0 | 0 | 0 | 0 | 0 | 0 | 10.175 |
| IMR322019070 | 0 | 0 | 0 | 0 | 0 | 0 | 5.247 |
| KIDNE1000145 | 0 | 0 | 19.453 | 0 | 0 | 0 | 1.808 |
| KIDNE2000085 | 4.436 | 0 | 0 | 0 | 0 | 0 | 0.672 |
| KIDNE2000252 | 1.843 | 0 | 0 | 0 | 0 | 0 | 3.352 |
| KIDNE2000266 | 0 | 0 | 0 | 0 | 0 | 0 | 2.752 |
| KIDNE2000403 | 1.782 | 18.349 | 0 | 0 | 0 | 0 | 1.620 |
| KIDNE2000497 | 0 | 0 | 0 | 0 | 0 | 21.454 | 0 |
| KIDNE2000517 | 0 | 0 | 0 | 0 | 0 | 21.431 | 0 |
| KIDNE2000574 | 0 | 0 | 0 | 0 | 0 | 5.121 | 0.489 |
| KIDNE2000665 | 4.175 | 0 | 0 | 0 | 0 | 0 | 0 |
| KIDNE2000947 | 0 | 0 | 0 | 0 | 0 | 0 | 3.304 |
| KIDNE2001162 | 0 | 0 | 3.283 | 0 | 0 | 0 | 0.610 |
| KIDNE2001467 | 1.124 | 0 | 0 | 0 | 0 | 0 | 2.045 |
| KIDNE2001847 | 0 | 0 | 0 | 0 | 0 | 0 | 2.379 |
| KIDNE2002252 | 0 | 0 | 0 | 0 | 0 | 0 | 14.250 |
| KIDNE2002883 | 4.892 | 0 | 0 | 0 | 0 | 0 | 0 |
| KIDNE2003305 | 0 | 0 | 17.763 | 0 | 0 | 0 | 0 |
| KIDNE2003316 | 0 | 0 | 53.223 | 0 | 0 | 0 | 0 |
| KIDNE2003357 | 31.890 | 0 | 0 | 0 | 0 | 0 | 0 |
| KIDNE2003941 | 0 | 0 | 0 | 0 | 0 | 0 | 0.330 |
| KIDNE2004295 | 0 | 0 | 0 | 0 | 0 | 43.680 | 0 |
| KIDNE2004864 | 0 | 0 | 0 | 0 | 0 | 0 | 6.382 |
| KIDNE2005676 | 0 | 0 | 0 | 0 | 0 | 0 | 3.679 |
| KIDNE2006009 | 0 | 0 | 0 | 0 | 0 | 0 | 9.640 |
| KIDNE2006014 | 0 | 0 | 0 | 0 | 0 | 20.315 | 1.941 |
| KIDNE2006030 | 0 | 0 | 15.012 | 0 | 0 | 0 | 1.395 |
| KIDNE2006248 | 0.671 | 0 | 0 | 0 | 0 | 2.127 | 0 |
| KIDNE2006760 | 0 | 0 | 0 | 0 | 0 | 0 | 4.410 |
| KIDNE2006775 | 0 | 0 | 12.888 | 0 | 0 | 0 | 2.395 |
| KIDNE2007328 | 3.816 | 0 | 0 | 0 | 0 | 0 | 0.578 |
| KIDNE2007352 | 0 | 0 | 0 | 0 | 0 | 0 | 0.543 |
| KIDNE2007910 | 19.546 | 0 | 0 | 0 | 0 | 0 | 0 |
| KIDNE2007954 | 0 | 0 | 29.658 | 0 | 0 | 0 | 0 |
| KIDNE2008022 | 0 | 0 | 0 | 0 | 0 | 0 | 15.790 |
| KIDNE2008048 | 2.314 | 0 | 3.774 | 0 | 0 | 0 | 0.351 |
| KIDNE2009367 | 0 | 0 | 0 | 0 | 0 | 0 | 1.478 |
| KIDNE2010750 | 0 | 0 | 0 | 0 | 0 | 0 | 13.925 |
| KIDNE2013346 | 0 | 0 | 0 | 0 | 0 | 0 | 27.273 |
| KIDNE2013845 | 2.411 | 0 | 0 | 0 | 0 | 0 | 0.731 |
| KIDNE2013866 | 0 | 0 | 0 | 0 | 0 | 0 | 15.790 |
| KIDNE2014290 | 1.512 | 0 | 9.863 | 0 | 0 | 0 | 3.208 |
| KIDNE2015710 | 0 | 0 | 0 | 39.504 | 0 | 0 | 0 |
| KIDNE2016570 | 0 | 0 | 0 | 0 | 0 | 0 | 15.790 |
| KIDNE2018217 | 0 | 0 | 0 | 0 | 0 | 41.518 | 0 |
| KIDNE2018352 | 0 | 0 | 0 | 0 | 0 | 0 | 9.324 |
| LIVER1000079 | 0 | 0 | 0 | 0 | 0 | 0 | 3.436 |
| LIVER1000126 | 0 | 0 | 0 | 0 | 0 | 0 | 5.011 |
| LIVER1000132 | 0 | 11.115 | 0 | 0 | 10.609 | 6.847 | 0.654 |
| LIVER1000542 | 0 | 0 | 0 | 0 | 0 | 0 | 6.883 |
| LIVER2000033 | 0 | 30.726 | 0 | 0 | 0 | 0 | 0 |
| LIVER2000416 | 0 | 0 | 0 | 0 | 0 | 0 | 1.479 |
| LIVER2000446 | 0 | 0 | 0 | 0 | 0 | 28.144 | 0 |
| LIVER2001113 | 0 | 0 | 0 | 0 | 0 | 0 | 7.012 |
| LIVER2001539 | 0 | 0 | 0 | 0 | 0 | 0 | 3.255 |
| LIVER2001608 | 0 | 0 | 0 | 0 | 0 | 8.985 | 0 |
| LIVER2002842 | 0 | 0 | 0 | 0 | 0 | 0 | 1.248 |
| LIVER2003581 | 0 | 0 | 0 | 0 | 70.979 | 0 | 0 |
| LIVER2005789 | 0 | 0 | 6.333 | 37.997 | 0 | 0 | 0 |
| LIVER2007783 | 4.425 | 0 | 0 | 0 | 0 | 0 | 0 |
| LYMPB1000141 | 0 | 0 | 0 | 0 | 0 | 0 | 2.800 |
| MAMGL1000032 | 0.695 | 0 | 1.133 | 6.801 | 0 | 1.102 | 0.421 |
| MAMGL1000035 | 0 | 0 | 0 | 0 | 0 | 0 | 0.658 |
| MAMGL1000100 | 0 | 0 | 0 | 0 | 0 | 0 | 0.193 |
| MAMGL1000173 | 1.143 | 0 | 0 | 0 | 0 | 0 | 0.347 |
| MESAN1000050 | 0 | 0 | 0 | 0 | 0 | 10.983 | 0 |
| MESAN1000080 | 3.079 | 0 | 0 | 0 | 0 | 0 | 1.867 |
| MESAN1000101 | 0 | 0 | 0 | 0 | 0 | 0 | 7.546 |
| MESAN2000092 | 0 | 0 | 0 | 0 | 0 | 0 | 5.784 |
| MESAN2000149 | 0 | 0 | 0 | 0 | 0 | 0 | 1.606 |
| MESAN2000267 | 0 | 0 | 0 | 0 | 0 | 0 | 0.268 |
| MESAN2000291 | 0 | 0 | 0 | 0 | 0 | 0 | 14.987 |
| MESAN2000434 | 0 | 0 | 0 | 0 | 0 | 0 | 14.987 |
| MESAN2000501 | 0 | 66.636 | 0 | 0 | 0 | 0 | 0 |
| MESAN2000909 | 0 | 0 | 0 | 0 | 0 | 0 | 3.610 |
| MESAN2001450 | 0 | 0 | 0 | 0 | 0 | 0 | 1.885 |
| MESAN2002086 | 0.898 | 0 | 0 | 0 | 0 | 2.848 | 0.272 |
| MESAN2002519 | 0 | 0 | 0 | 0 | 0 | 31.731 | 3.032 |
| MESAN2002724 | 25.857 | 0 | 0 | 0 | 0 | 0 | 0.603 |
| MESAN2002790 | 10.550 | 0 | 0 | 0 | 0 | 0 | 0 |
| MESAN2002844 | 0 | 0 | 0 | 0 | 0 | 0 | 4.737 |
| MESAN2002940 | 0 | 0 | 0 | 0 | 0 | 0 | 20.789 |
| MESAN2003444 | 8.035 | 0 | 0 | 0 | 0 | 0 | 4.870 |
| MESAN2003622 | 0 | 0 | 0 | 0 | 0 | 0 | 0.393 |
| MESAN2005633 | 0 | 0 | 0 | 0 | 0 | 0 | 11.221 |
| MESAN2005689 | 0 | 0 | 0 | 0 | 0 | 0 | 4.056 |
| MESAN2005766 | 0 | 0 | 6.937 | 0 | 0 | 0 | 2.578 |
| MESAN2005958 | 5.808 | 0 | 0 | 0 | 0 | 0 | 1.760 |
| MESAN2006563 | 0 | 0 | 0 | 0 | 0 | 0 | 0.451 |
| MESAN2007755 | 0 | 0 | 0 | 0 | 0 | 0 | 5.980 |
| MESAN2008679 | 0 | 0 | 49.292 | 0 | 0 | 0 | 0 |
| MESAN2009580 | 0 | 24.667 | 0 | 0 | 0 | 0 | 0 |
| MESAN2011984 | 3.680 | 0 | 0 | 0 | 0 | 0 | 1.115 |
| MESAN2012054 | 0 | 0 | 35.889 | 0 | 0 | 0 | 1.667 |
| MESAN2013284 | 0 | 0 | 0 | 0 | 0 | 0 | 0.803 |
| MESAN2014412 | 0 | 0 | 0 | 0 | 0 | 0 | 12.581 |
| MESAN2015515 | 0 | 0 | 0 | 0 | 0 | 0 | 1.736 |
| MESAN2016159 | 0 | 0 | 0 | 0 | 0 | 0 | 14.987 |
| NB9N41000121 | 0 | 0 | 0 | 0 | 0 | 5.138 | 1.473 |
| NB9N41000340 | 3.128 | 0 | 0 | 0 | 0 | 0 | 0 |
| NB9N42000104 | 0 | 0 | 0 | 0 | 0 | 0 | 1.626 |
| NB9N42000342 | 0 | 16.015 | 0 | 0 | 15.287 | 4.933 | 0 |
| NHNPC2000187 | 2.928 | 0 | 6.367 | 9.550 | 0 | 0 | 0 |
| NHNPC2000206 | 0 | 0 | 0 | 0 | 0 | 0 | 2.244 |
| NHNPC2000212 | 0 | 0 | 0 | 0 | 0 | 0 | 2.278 |
| NHNPC2001108 | 0 | 0 | 0 | 0 | 0 | 0 | 2.553 |
| NOVAR1000091 | 0 | 0 | 27.245 | 0 | 0 | 0 | 0 |
| NOVAR1000102 | 0 | 0 | 0 | 0 | 0 | 0 | 0.906 |
| NOVAR2000783 | 0 | 0 | 0 | 0 | 0 | 0 | 2.472 |
| NT2NE1000014 | 18.748 | 0 | 0 | 0 | 0 | 0 | 0 |
| NT2NE1000018 | 0 | 0 | 0 | 0 | 0 | 0 | 11.378 |
| NT2NE1000163 | 0 | 0 | 2.849 | 0 | 0 | 0 | 0.265 |
| NT2NE2000056 | 0 | 0 | 0 | 0 | 0 | 0 | 1.027 |
| NT2NE2000174 | 0 | 0 | 2.684 | 0 | 0 | 0 | 0.998 |
| NT2NE2000195 | 0 | 0 | 0 | 0 | 32.590 | 0 | 1.005 |
| NT2NE2000259 | 0 | 0 | 0 | 0 | 0 | 0 | 5.516 |
| NT2NE2000260 | 0 | 0 | 0.850 | 5.099 | 0 | 2.479 | 0 |
| NT2NE2000327 | 0 | 0 | 0 | 0 | 0 | 0 | 27.955 |
| NT2NE2000383 | 0 | 0 | 0 | 0 | 0 | 10.396 | 0.993 |
| NT2NE2000384 | 0 | 0 | 0 | 0 | 0 | 15.747 | 3.009 |
| NT2NE2000575 | 0 | 5.340 | 1.691 | 0 | 0 | 3.290 | 0.943 |
| NT2NE2000707 | 0.722 | 7.433 | 2.354 | 0 | 0 | 0 | 0.656 |
| NT2NE2000763 | 0 | 0 | 0 | 0 | 0 | 0 | 2.353 |
| NT2NE2000809 | 0 | 0 | 0 | 0 | 0 | 0 | 2.216 |
| NT2NE2000950 | 0 | 0 | 0 | 0 | 0 | 0 | 6.852 |
| NT2NE2000963 | 1.484 | 5.095 | 1.614 | 0 | 4.864 | 0 | 0.450 |
| NT2NE2001005 | 0 | 0 | 0 | 0 | 0 | 0 | 15.281 |
| NT2NE2001048 | 0 | 0 | 0 | 0 | 0 | 0 | 1.957 |
| NT2NE2001176 | 2.463 | 0 | 0 | 0 | 0 | 0 | 0 |
| NT2NE2001435 | 0 | 0 | 0 | 0 | 0 | 0 | 2.085 |
| NT2NE2001545 | 0 | 0 | 0 | 0 | 0 | 0 | 2.342 |
| NT2NE2001598 | 0 | 0 | 0 | 0 | 0 | 0 | 8.292 |
| NT2NE2001648 | 0 | 0 | 0 | 0 | 0 | 0 | 5.673 |
| NT2NE2001697 | 0 | 0 | 0 | 0 | 0 | 74.712 | 0 |
| NT2NE2001793 | 2.237 | 0 | 0 | 0 | 0 | 0 | 6.780 |
| NT2NE2002870 | 0 | 0 | 0 | 0 | 0 | 0 | 2.984 |
| NT2NE2003569 | 0 | 0 | 0 | 0 | 0 | 0 | 2.269 |
| NT2NE2003887 | 0 | 0 | 0 | 0 | 0 | 0 | 4.080 |
| NT2NE2003921 | 0 | 0 | 0 | 0 | 0 | 0 | 3.988 |
| NT2NE2004255 | 0 | 0 | 0 | 0 | 0 | 0 | 7.787 |
| NT2NE2005419 | 0 | 0 | 0 | 0 | 0 | 0 | 7.569 |
| NT2NE2005688 | 0 | 0 | 0 | 0 | 0 | 0 | 0.976 |
| NT2NE2006478 | 0 | 0 | 0 | 0 | 0 | 0 | 7.024 |
| NT2NE2006909 | 0 | 0 | 0 | 0 | 0 | 0 | 0.559 |
| NT2NE2008785 | 0 | 0 | 65.999 | 0 | 0 | 0 | 0 |
| NT2NE2010842 | 0 | 0 | 0 | 0 | 0 | 0 | 0.616 |
| NT2NE2010854 | 0 | 0 | 0 | 0 | 0 | 0 | 15.281 |
| NT2NE2011107 | 0 | 0 | 0 | 0 | 0 | 0 | 31.446 |
| NT2NE2011485 | 0 | 0 | 0 | 0 | 0 | 0 | 12.386 |
| NT2NE2012790 | 0 | 0 | 0 | 0 | 0 | 0 | 3.921 |
| NT2NE2014104 | 0 | 0 | 0 | 0 | 0 | 11.484 | 0 |
| NT2NE2017982 | 0 | 0 | 0 | 0 | 0 | 0 | 15.281 |
| NT2NE2018180 | 8.110 | 0 | 0 | 0 | 0 | 0 | 4.916 |
| NT2NE2018490 | 0 | 0 | 0 | 0 | 0 | 0 | 15.281 |
| NT2NE2019091 | 0 | 0 | 0 | 0 | 0 | 12.953 | 0 |
| NT2RI1000027 | 0 | 44.915 | 0 | 0 | 0 | 0 | 0 |
| NT2RI1000048 | 1.831 | 0 | 0 | 0 | 0 | 0 | 0 |
| NT2RI2000064 | 1.161 | 0 | 0 | 0 | 0 | 0 | 1.056 |
| NT2RI2000107 | 0 | 0 | 0 | 0 | 0 | 0 | 4.542 |
| NT2RI2000133 | 0 | 0 | 0 | 0 | 54.324 | 0 | 0 |
| NT2RI2000255 | 0 | 0 | 0 | 0 | 0 | 0 | 0.782 |
| NT2RI2000263 | 0 | 0 | 0 | 0 | 0 | 0 | 2.236 |
| NT2RI2000284 | 1.966 | 0 | 0 | 0 | 0 | 0 | 0.298 |
| NT2RI2000294 | 0 | 0 | 0 | 0 | 0 | 0 | 3.712 |
| NT2RI2000412 | 0 | 0 | 0 | 0 | 0 | 0 | 5.038 |
| NT2RI2000421 | 0 | 0 | 0 | 0 | 0 | 0 | 1.989 |
| NT2RI2000578 | 0 | 0 | 0 | 0 | 0 | 2.288 | 0.874 |
| NT2RI2000685 | 0 | 0 | 6.970 | 0 | 0 | 0 | 8.419 |
| NT2RI2000689 | 0 | 0 | 21.755 | 0 | 0 | 0 | 0 |
| NT2RI2000727 | 0.558 | 0 | 5.460 | 5.460 | 10.970 | 3.540 | 0.338 |
| NT2RI2001091 | 0 | 0 | 0 | 0 | 0 | 0 | 2.298 |
| NT2RI2001167 | 0 | 0 | 0 | 0 | 0 | 0 | 26.455 |
| NT2RI2001230 | 1.664 | 0 | 0 | 0 | 0 | 0 | 0 |
| NT2RI2001342 | 0 | 35.059 | 0 | 0 | 0 | 0 | 0 |
| NT2RI2001422 | 0 | 0 | 0 | 0 | 0 | 0 | 2.430 |
| NT2RI2001540 | 0.859 | 1.264 | 0.801 | 1.201 | 2.414 | 1.168 | 0.707 |
| NT2RI2001621 | 0 | 0 | 0 | 0 | 0 | 0 | 6.003 |
| NT2RI2001657 | 2.308 | 0 | 0 | 0 | 0 | 0 | 0.350 |
| NT2RI2001731 | 0 | 0 | 0 | 0 | 0 | 0 | 1.238 |
| NT2RI2001859 | 0 | 0 | 13.653 | 0 | 0 | 0 | 3.172 |
| NT2RI2001910 | 16.109 | 0 | 0 | 0 | 0 | 0 | 9.765 |
| NT2RI2002091 | 1.215 | 1.192 | 1.698 | 0.849 | 0.853 | 0.734 | 0.123 |
| NT2RI2002117 | 0 | 0 | 0 | 0 | 0 | 0 | 3.080 |
| NT2RI2002152 | 0 | 0 | 0 | 0 | 0 | 0 | 0.822 |
| NT2RI2002243 | 14.189 | 0 | 0 | 0 | 0 | 0 | 0 |
| NT2RI2002266 | 0 | 0 | 0 | 0 | 0 | 0 | 3.277 |
| NT2RI2002316 | 0 | 0 | 11.280 | 0 | 0 | 0 | 0 |
| NT2RI2002554 | 3.911 | 0 | 0 | 0 | 0 | 0 | 3.556 |
| NT2RI2002585 | 0 | 0 | 0 | 0 | 0 | 0 | 50.993 |
| NT2RI2002654 | 0 | 0 | 4.133 | 0 | 0 | 0 | 0 |
| NT2RI2002802 | 0 | 0 | 0 | 0 | 0 | 0 | 18.792 |
| NT2RI2002847 | 0 | 0 | 0 | 0 | 0 | 8.588 | 3.282 |
| NT2RI2002852 | 0 | 0 | 0 | 0 | 0 | 0 | 26.455 |
| NT2RI2003019 | 0 | 0 | 0 | 0 | 0 | 0 | 3.419 |
| NT2RI2003051 | 6.624 | 0 | 0 | 0 | 0 | 0 | 6.692 |
| NT2RI2003205 | 0 | 0 | 0 | 0 | 0 | 22.024 | 0 |
| NT2RI2003304 | 0 | 5.829 | 1.846 | 0 | 0 | 0 | 0.515 |
| NT2RI2003317 | 0 | 0 | 0 | 0 | 0 | 0 | 2.061 |
| NT2RI2003338 | 0 | 0 | 0 | 0 | 0 | 0 | 0.662 |
| NT2RI2003344 | 0 | 0 | 0 | 0 | 0 | 0 | 22.913 |
| NT2RI2003399 | 0 | 0 | 0 | 0 | 0 | 0 | 41.841 |
| NT2RI2003420 | 0 | 0 | 0 | 0 | 0 | 0 | 1.007 |
| NT2RI2003556 | 0 | 0 | 0 | 0 | 0 | 0 | 0.995 |
| NT2RI2003678 | 8.213 | 42.290 | 0 | 0 | 0 | 0 | 1.245 |
| NT2RI2003695 | 0 | 0 | 0 | 16.618 | 0 | 0 | 0.515 |
| NT2RI2003738 | 0 | 0 | 2.461 | 0 | 0 | 0 | 0.114 |
| NT2RI2003921 | 0 | 0 | 0 | 0 | 0 | 0 | 0.310 |
| NT2RI2003993 | 0 | 0 | 0 | 0 | 0 | 0 | 2.482 |
| NT2RI2004188 | 0 | 0 | 0 | 0 | 0 | 0 | 5.631 |
| NT2RI2004209 | 15.835 | 0 | 0 | 0 | 0 | 0 | 0 |
| NT2RI2004398 | 0 | 0 | 0 | 0 | 0 | 0 | 3.256 |
| NT2RI2004410 | 0 | 0 | 0 | 0 | 0 | 0 | 4.891 |
| NT2RI2004535 | 0 | 0 | 0 | 0 | 0 | 0 | 3.754 |
| NT2RI2004608 | 0 | 0 | 0 | 0 | 0 | 0 | 7.255 |
| NT2RI2005061 | 9.409 | 0 | 0 | 0 | 0 | 0 | 2.852 |
| NT2RI2005096 | 0 | 0 | 0 | 0 | 0 | 0 | 26.455 |
| NT2RI2005116 | 11.028 | 0 | 0 | 0 | 0 | 0 | 0.836 |
| NT2RI2005166 | 0 | 0 | 0 | 0 | 0 | 0 | 34.096 |
| NT2RI2005335 | 2.505 | 0 | 0 | 0 | 0 | 0 | 4.555 |
| NT2RI2005405 | 0.605 | 12.470 | 1.975 | 0 | 0 | 1.921 | 0.367 |
| NT2RI2005647 | 0 | 0 | 0 | 0 | 0 | 9.049 | 0 |
| NT2RI2005713 | 0 | 0 | 0 | 0 | 0 | 0 | 0.967 |
| NT2RI2005723 | 1.082 | 0 | 0 | 0 | 0 | 0 | 2.296 |
| NT2RI2005811 | 9.913 | 0 | 0 | 0 | 0 | 0 | 0 |
| NT2RI2005818 | 5.493 | 0 | 0 | 0 | 0 | 0 | 0 |
| NT2RI2006070 | 0 | 0 | 0 | 0 | 0 | 0 | 2.687 |
| NT2RI2006072 | 1.650 | 0 | 0 | 0 | 0 | 0 | 0.500 |
| NT2RI2006412 | 0 | 0 | 0 | 0 | 0 | 0 | 0.536 |
| NT2RI2006506 | 0 | 0 | 0 | 0 | 0 | 0 | 8.806 |
| NT2RI2006553 | 0 | 0 | 26.018 | 0 | 0 | 0 | 0 |
| NT2RI2006647 | 0 | 0 | 0 | 0 | 0 | 0 | 2.997 |
| NT2RI2006735 | 0 | 0 | 0 | 0 | 0 | 0 | 10.288 |
| NT2RI2006825 | 0 | 0 | 0 | 0 | 0 | 0 | 1.792 |
| NT2RI2006855 | 0 | 0 | 0 | 0 | 0 | 0 | 11.270 |
| NT2RI2006856 | 0 | 0 | 0 | 0 | 0 | 0 | 1.531 |
| NT2RI2006943 | 0 | 0 | 0 | 25.667 | 0 | 0 | 0 |
| NT2RI2007054 | 0 | 0 | 0 | 0 | 0 | 0 | 5.941 |
| NT2RI2007133 | 0 | 0 | 0 | 0 | 0 | 0 | 0.256 |
| NT2RI2007148 | 0 | 0 | 0 | 0 | 0 | 0 | 0.617 |
| NT2RI2007384 | 0.934 | 9.619 | 0 | 0 | 0 | 2.963 | 0.849 |
| NT2RI2007445 | 0 | 0 | 0 | 37.840 | 0 | 12.268 | 0 |
| NT2RI2007468 | 0 | 0 | 29.393 | 0 | 0 | 0 | 2.731 |
| NT2RI2007469 | 0 | 0 | 0 | 0 | 0 | 0 | 41.841 |
| NT2RI2007498 | 0 | 9.058 | 0 | 8.606 | 0 | 0 | 0.267 |
| NT2RI2007572 | 3.223 | 0 | 0 | 0 | 0 | 5.112 | 2.930 |
| NT2RI2007589 | 0 | 0 | 0 | 0 | 0 | 0 | 2.792 |
| NT2RI2007723 | 7.630 | 0 | 0 | 0 | 0 | 0 | 0 |
| NT2RI2007729 | 0 | 0 | 0 | 0 | 0 | 79.014 | 0 |
| NT2RI2007853 | 0 | 0 | 15.750 | 0 | 0 | 0 | 0 |
| NT2RI2007879 | 0 | 0 | 0 | 0 | 0 | 10.859 | 0 |
| NT2RI2008007 | 41.744 | 0 | 0 | 0 | 0 | 0 | 0 |
| NT2RI2008045 | 0 | 17.246 | 0 | 0 | 0 | 15.937 | 0 |
| NT2RI2008171 | 0 | 0 | 0 | 0 | 0 | 0 | 5.805 |
| NT2RI2008188 | 0 | 0 | 0 | 0 | 0 | 0 | 18.792 |
| NT2RI2008336 | 1.728 | 8.898 | 5.636 | 0 | 0 | 0 | 0.524 |
| NT2RI2008396 | 2.852 | 0 | 0 | 0 | 9.345 | 0 | 0.576 |
| NT2RI2008526 | 0 | 0 | 5.393 | 0 | 0 | 0 | 0 |
| NT2RI2008656 | 0 | 0 | 0 | 0 | 0 | 0 | 1.775 |
| NT2RI2008724 | 0 | 0 | 0 | 0 | 0 | 0 | 2.401 |
| NT2RI2008749 | 0 | 0 | 0 | 0 | 0 | 0 | 0.370 |
| NT2RI2008808 | 0 | 0 | 0 | 0 | 0 | 0 | 2.963 |
| NT2RI2008942 | 2.262 | 0 | 7.377 | 0 | 0 | 0 | 0.685 |
| NT2RI2009037 | 0 | 0 | 0 | 0 | 0 | 0 | 4.960 |
| NT2RI2009065 | 1.007 | 0 | 0 | 0 | 9.902 | 0 | 0.305 |
| NT2RI2009066 | 0 | 0 | 0 | 0 | 0 | 0 | 16.125 |
| NT2RI2009083 | 0 | 0 | 0 | 0 | 0 | 0 | 26.810 |
| NT2RI2009103 | 0 | 0 | 0 | 0 | 0 | 0 | 41.841 |
| NT2RI2009173 | 0 | 0 | 7.846 | 0 | 0 | 0 | 1.458 |
| NT2RI2009233 | 0 | 0 | 0 | 0 | 0 | 0 | 4.423 |
| NT2RI2009281 | 0 | 0 | 0 | 0 | 0 | 0 | 0.920 |
| NT2RI2009583 | 0 | 0 | 0 | 9.443 | 4.744 | 1.531 | 0.146 |
| NT2RI2012350 | 0.863 | 0 | 0 | 0 | 0 | 0 | 0.262 |
| NT2RI2012726 | 0 | 0 | 0 | 0 | 0 | 0 | 9.097 |
| NT2RI2013357 | 0 | 0 | 0 | 0 | 0 | 0 | 26.455 |
| NT2RI2015342 | 0 | 0 | 0 | 0 | 0 | 10.293 | 0.983 |
| NT2RI2015533 | 0 | 0 | 0 | 0 | 0 | 0 | 4.064 |
| NT2RI2018448 | 0 | 0 | 0 | 0 | 0 | 0 | 4.116 |
| NT2RI2024313 | 0 | 0 | 0 | 0 | 0 | 0 | 28.733 |
| NT2RI2024935 | 0 | 0 | 0 | 0 | 92.105 | 0 | 0 |
| NT2RI3001515 | 0 | 0 | 0 | 0 | 0 | 0 | 7.080 |
| NT2RI3003104 | 0 | 0 | 0 | 0 | 0 | 0 | 71.575 |
| NT2RI3005724 | 0 | 0 | 0 | 0 | 0 | 0 | 7.748 |
| NT2RI3006284 | 8.925 | 0 | 0 | 0 | 0 | 14.156 | 0 |
| NT2RI3006340 | 0 | 0 | 0 | 0 | 0 | 0 | 10.676 |
| NT2RI3007543 | 3.660 | 0 | 0 | 0 | 0 | 0 | 1.109 |
| NT2RI3009158 | 54.273 | 0 | 0 | 0 | 0 | 0 | 0 |
| NT2RP6000017 | 0 | 0 | 0 | 0 | 0 | 0 | 1.956 |
| NT2RP6000039 | 0 | 0 | 0 | 0 | 0 | 0 | 3.516 |
| NT2RP6000043 | 0 | 0 | 26.719 | 0 | 0 | 0 | 0 |
| NT2RP6000072 | 0 | 0 | 11.367 | 0 | 0 | 0 | 1.056 |
| NT2RP6000078 | 6.204 | 0 | 0 | 0 | 0 | 0 | 0 |
| NT2RP6000085 | 0 | 0 | 0 | 0 | 0 | 0 | 27.209 |
| NT2RP6000100 | 47.227 | 0 | 0 | 0 | 0 | 0 | 0 |
| NT2RP6000109 | 4.119 | 0 | 0 | 0 | 0 | 0 | 2.497 |
| NT2RP6000110 | 0 | 0 | 0 | 0 | 0 | 0 | 1.790 |
| NT2RP6000123 | 1.689 | 0 | 0 | 0 | 0 | 0 | 0.768 |
| NT2RP6000130 | 0 | 0 | 0 | 0 | 0 | 0 | 5.232 |
| NT2RP7000069 | 7.265 | 0 | 0 | 0 | 0 | 0 | 13.211 |
| NT2RP7000076 | 0 | 0 | 0 | 0 | 0 | 8.339 | 1.593 |
| NT2RP7000173 | 0 | 12.348 | 0 | 0 | 0 | 0 | 5.088 |
| NT2RP7000238 | 2.792 | 0 | 0 | 0 | 0 | 0 | 0.846 |
| NT2RP7000259 | 0 | 0 | 0 | 0 | 0 | 0 | 8.741 |
| NT2RP7000271 | 0 | 0 | 0 | 0 | 0 | 0 | 3.965 |
| NT2RP7000477 | 0 | 0 | 0 | 0 | 0 | 0 | 1.293 |
| NT2RP7000586 | 0 | 0 | 0 | 0 | 0 | 0 | 7.182 |
| NT2RP7000600 | 4.779 | 0 | 0 | 0 | 0 | 0 | 4.346 |
| NT2RP7000624 | 0 | 0 | 0 | 0 | 0 | 0 | 2.852 |
| NT2RP7000906 | 5.627 | 0 | 0 | 0 | 0 | 0 | 5.969 |
| NT2RP7001080 | 0 | 0 | 0 | 0 | 0 | 0 | 21.879 |
| NT2RP7001156 | 0 | 0 | 0 | 0 | 0 | 0 | 21.336 |
| NT2RP7001166 | 0 | 0 | 5.261 | 0 | 0 | 0 | 4.400 |
| NT2RP7001231 | 0 | 0 | 0 | 0 | 0 | 0 | 2.700 |
| NT2RP7001283 | 0 | 0 | 3.960 | 0 | 0 | 0 | 4.047 |
| NT2RP7001678 | 33.546 | 0 | 0 | 0 | 0 | 0 | 0 |
| NT2RP7001962 | 0 | 0 | 0 | 0 | 0 | 0 | 3.473 |
| NT2RP7002028 | 4.334 | 0 | 0 | 0 | 0 | 0 | 1.314 |
| NT2RP7002379 | 5.429 | 11.181 | 0 | 0 | 0 | 0 | 2.303 |
| NT2RP7002449 | 0 | 0 | 0 | 0 | 0 | 0 | 10.564 |
| NT2RP7002450 | 0 | 0 | 0 | 0 | 0 | 0 | 1.745 |
| NT2RP7002619 | 2.350 | 0 | 0 | 0 | 0 | 7.455 | 2.137 |
| NT2RP7002738 | 0 | 0 | 0 | 0 | 0 | 0 | 3.763 |
| NT2RP7002779 | 0 | 0 | 0 | 0 | 0 | 0 | 21.336 |
| NT2RP7002802 | 7.882 | 0 | 0 | 0 | 0 | 0 | 4.778 |
| NT2RP7002906 | 0 | 0 | 0 | 0 | 0 | 0 | 1.833 |
| NT2RP7002982 | 0 | 0 | 0 | 0 | 0 | 0 | 2.541 |
| NT2RP7003203 | 3.984 | 0 | 0 | 0 | 0 | 0 | 2.415 |
| NT2RP7003261 | 0 | 0 | 0 | 0 | 0 | 0 | 0.888 |
| NT2RP7003319 | 0 | 0 | 0 | 0 | 0 | 0 | 1.193 |
| NT2RP7003629 | 0 | 0 | 0 | 0 | 0 | 0 | 7.396 |
| NT2RP7003647 | 2.538 | 0 | 8.279 | 0 | 0 | 0 | 3.847 |
| NT2RP7003680 | 0 | 0 | 0 | 0 | 0 | 0 | 14.629 |
| NT2RP7003688 | 3.185 | 0 | 0 | 0 | 0 | 5.052 | 3.379 |
| NT2RP7003724 | 0 | 0 | 0 | 0 | 0 | 0 | 12.073 |
| NT2RP7003960 | 0 | 0 | 0 | 0 | 35.126 | 0 | 4.332 |
| NT2RP7004173 | 0 | 0 | 0 | 0 | 0 | 0 | 0.499 |
| NT2RP7004196 | 0 | 0 | 9.217 | 0 | 0 | 0 | 0 |
| NT2RP7004204 | 7.355 | 37.874 | 0 | 0 | 0 | 0 | 2.229 |
| NT2RP7004233 | 0 | 0 | 0 | 0 | 0 | 0 | 15.633 |
| NT2RP7004260 | 0 | 0 | 0 | 0 | 0 | 0 | 16.771 |
| NT2RP7004352 | 0 | 0 | 0 | 0 | 0 | 0 | 4.441 |
| NT2RP7004373 | 5.077 | 0 | 0 | 0 | 0 | 0 | 2.308 |
| NT2RP7004641 | 0 | 0 | 0 | 0 | 0 | 0 | 16.771 |
| NT2RP7004687 | 0 | 0 | 0 | 0 | 0 | 0 | 12.324 |
| NT2RP7004728 | 0 | 0 | 0 | 0 | 0 | 0 | 2.055 |
| NT2RP7004751 | 0 | 0 | 3.293 | 0 | 0 | 3.202 | 0 |
| NT2RP7004911 | 3.802 | 0 | 0 | 0 | 0 | 0 | 5.761 |
| NT2RP7004946 | 1.857 | 0 | 0 | 0 | 0 | 0 | 3.939 |
| NT2RP7005529 | 0 | 0 | 0 | 0 | 0 | 0 | 4.826 |
| NT2RP7005675 | 0 | 0 | 0 | 0 | 0 | 0 | 9.505 |
| NT2RP7005750 | 0 | 0 | 0 | 0 | 0 | 0 | 4.678 |
| NT2RP7006033 | 0 | 0 | 0 | 0 | 0 | 0 | 5.025 |
| NT2RP7006188 | 5.615 | 0 | 0 | 0 | 0 | 0 | 5.105 |
| NT2RP7006223 | 0 | 0 | 0 | 0 | 0 | 0 | 2.762 |
| NT2RP7006296 | 0 | 0 | 0 | 0 | 0 | 0 | 21.336 |
| NT2RP7006304 | 0 | 0 | 0 | 0 | 0 | 0 | 21.336 |
| NT2RP7006539 | 0 | 0 | 0 | 0 | 0 | 39.810 | 0 |
| NT2RP7006547 | 0 | 0 | 0 | 0 | 0 | 0 | 21.336 |
| NT2RP7006717 | 4.257 | 0 | 0 | 0 | 13.948 | 0 | 2.150 |
| NT2RP7007114 | 0 | 0 | 0 | 0 | 0 | 0 | 10.709 |
| NT2RP7007154 | 1.230 | 0 | 0 | 0 | 0 | 0 | 0 |
| NT2RP7007252 | 0 | 0 | 0 | 0 | 0 | 0 | 22.444 |
| NT2RP7007310 | 8.032 | 0 | 0 | 0 | 0 | 0 | 0 |
| NT2RP7007381 | 0 | 0 | 0 | 0 | 0 | 0 | 10.251 |
| NT2RP7007387 | 8.854 | 0 | 0 | 0 | 0 | 0 | 1.342 |
| NT2RP7007537 | 0 | 0 | 0 | 0 | 0 | 0 | 3.215 |
| NT2RP7007580 | 0 | 0 | 0 | 0 | 0 | 0 | 15.624 |
| NT2RP7007643 | 0 | 0 | 0 | 0 | 0 | 0 | 21.336 |
| NT2RP7007842 | 0 | 0 | 0 | 0 | 0 | 0 | 1.254 |
| NT2RP7007930 | 0 | 0 | 0 | 0 | 0 | 0 | 21.336 |
| NT2RP7008167 | 0 | 0 | 0 | 0 | 0 | 0 | 8.306 |
| NT2RP7008190 | 0 | 0 | 0 | 0 | 0 | 9.076 | 3.468 |
| NT2RP7008315 | 0 | 0 | 52.791 | 0 | 0 | 0 | 0 |
| NT2RP7008543 | 11.867 | 0 | 0 | 0 | 0 | 0 | 0 |
| NT2RP7009087 | 0 | 0 | 0 | 0 | 0 | 0 | 4.764 |
| NT2RP7009108 | 1.917 | 0 | 0 | 0 | 0 | 0 | 0 |
| NT2RP7009147 | 0 | 0 | 11.206 | 0 | 0 | 0 | 1.041 |
| NT2RP7009149 | 2.580 | 0 | 0 | 0 | 0 | 0 | 0 |
| NT2RP7009259 | 0 | 0 | 0 | 0 | 0 | 0 | 1.469 |
| NT2RP7009322 | 0 | 0 | 0 | 0 | 0 | 73.950 | 0 |
| NT2RP7009370 | 0 | 0 | 0 | 0 | 0 | 0 | 21.336 |
| NT2RP7009373 | 0 | 0 | 0 | 0 | 0 | 0 | 2.626 |
| NT2RP7009394 | 0 | 0 | 0 | 0 | 25.636 | 0 | 5.533 |
| NT2RP7009481 | 0 | 0 | 0 | 0 | 0 | 0 | 16.063 |
| NT2RP7009482 | 1.000 | 0 | 0 | 0 | 0 | 0 | 0.606 |
| NT2RP7014910 | 0 | 0 | 0 | 0 | 0 | 0 | 1.563 |
| NT2RP7020123 | 0 | 0 | 0 | 89.750 | 0 | 0 | 0 |
| NT2RP8000435 | 0 | 0 | 0 | 0 | 0 | 0 | 8.722 |
| NT2RP8007920 | 4.267 | 0 | 0 | 0 | 0 | 0 | 1.293 |
| NTONG1000033 | 2.643 | 0 | 0 | 0 | 0 | 0 | 4.004 |
| NTONG1000098 | 1.374 | 0 | 0 | 0 | 0 | 0 | 11.245 |
| NTONG1000246 | 0 | 0 | 0 | 0 | 0 | 0 | 6.133 |
| NTONG2000265 | 0 | 0 | 0 | 0 | 0 | 0 | 5.015 |
| NTONG2000334 | 0 | 0 | 0 | 0 | 0 | 0 | 1.336 |
| NTONG2000413 | 0 | 0 | 0 | 0 | 0 | 0 | 7.055 |
| NTONG2000800 | 0 | 0 | 0 | 0 | 0 | 21.737 | 0 |
| NTONG2000878 | 0 | 0 | 8.307 | 0 | 0 | 0 | 0 |
| NTONG2000977 | 2.120 | 0 | 0 | 0 | 0 | 6.726 | 0 |
| NTONG2000985 | 0 | 0 | 0 | 0 | 0 | 0 | 1.219 |
| NTONG2001137 | 2.468 | 25.420 | 0 | 0 | 0 | 7.830 | 0 |
| NTONG2001222 | 0 | 0 | 0 | 0 | 0 | 17.020 | 0 |
| NTONG2001550 | 0 | 0 | 0 | 0 | 0 | 0 | 2.275 |
| NTONG2001567 | 0.661 | 0 | 0 | 0 | 0 | 2.098 | 0.200 |
| NTONG2001587 | 0 | 0 | 0 | 0 | 0 | 12.221 | 0 |
| NTONG2001762 | 3.889 | 0 | 0 | 0 | 0 | 0 | 7.072 |
| NTONG2002582 | 0 | 0 | 0 | 0 | 0 | 0 | 9.834 |
| NTONG2003454 | 0 | 0 | 0 | 0 | 0 | 0 | 1.949 |
| NTONG2003928 | 0 | 0 | 0 | 0 | 0 | 0 | 3.808 |
| NTONG2004844 | 0 | 0 | 0 | 0 | 0 | 0 | 1.059 |
| NTONG2005391 | 0 | 0 | 0 | 0 | 0 | 0 | 7.619 |
| NTONG2005520 | 2.871 | 0 | 0 | 0 | 0 | 4.554 | 2.176 |
| NTONG2005801 | 0 | 0 | 25.065 | 0 | 0 | 0 | 5.823 |
| NTONG2006301 | 11.854 | 0 | 0 | 0 | 0 | 0 | 0 |
| NTONG2006484 | 0 | 0 | 0 | 0 | 0 | 29.574 | 0 |
| NTONG2007658 | 8.632 | 0 | 0 | 0 | 0 | 0 | 0 |
| NTONG2007693 | 0 | 0 | 0 | 0 | 0 | 48.142 | 0 |
| NTONG2008088 | 0 | 0 | 0 | 0 | 0 | 0 | 13.359 |
| NTONG2008483 | 0 | 0 | 0 | 0 | 0 | 0 | 3.133 |
| NTONG2008944 | 0 | 0 | 0 | 0 | 0 | 0 | 7.635 |
| NTONG2009576 | 15.325 | 0 | 0 | 0 | 0 | 0 | 0 |
| OCBBF1000086 | 0 | 0 | 0 | 0 | 0 | 0 | 1.001 |
| OCBBF1000185 | 18.160 | 0 | 0 | 0 | 0 | 0 | 2.752 |
| OCBBF1000188 | 2.246 | 0 | 0 | 0 | 0 | 0 | 2.042 |
| OCBBF1000315 | 0 | 0 | 0 | 0 | 0 | 0 | 22.877 |
| OCBBF2000013 | 4.197 | 0 | 0 | 0 | 0 | 0 | 0 |
| OCBBF2000015 | 0 | 0 | 0 | 0 | 0 | 0 | 0.517 |
| OCBBF2000467 | 0 | 0 | 0 | 0 | 0 | 0 | 1.318 |
| OCBBF2000831 | 0 | 0 | 0 | 0 | 0 | 0 | 9.442 |
| OCBBF2000846 | 0 | 0 | 0 | 0 | 15.515 | 0 | 0 |
| OCBBF2000982 | 0 | 0 | 0 | 0 | 0 | 0 | 1.710 |
| OCBBF2000986 | 6.235 | 0 | 6.779 | 0 | 0 | 0 | 0.630 |
| OCBBF2001140 | 0 | 0 | 0 | 0 | 0 | 0 | 18.479 |
| OCBBF2001166 | 0 | 0 | 0 | 0 | 0 | 0 | 3.583 |
| OCBBF2001186 | 38.181 | 0 | 0 | 0 | 0 | 0 | 0 |
| OCBBF2001210 | 0 | 0 | 0 | 0 | 0 | 0 | 4.740 |
| OCBBF2001252 | 0 | 0 | 0 | 0 | 0 | 0 | 3.009 |
| OCBBF2001408 | 0 | 0 | 0 | 0 | 0 | 0 | 12.622 |
| OCBBF2001416 | 0 | 0 | 0 | 0 | 0 | 0 | 27.283 |
| OCBBF2001492 | 0 | 0 | 9.653 | 0 | 0 | 0 | 0 |
| OCBBF2001528 | 0 | 0 | 0 | 0 | 0 | 0 | 21.673 |
| OCBBF2001586 | 0 | 0 | 0 | 0 | 0 | 0 | 2.344 |
| OCBBF2001687 | 0 | 0 | 0 | 0 | 0 | 0 | 6.287 |
| OCBBF2001794 | 0 | 0 | 0 | 0 | 0 | 0 | 15.452 |
| OCBBF2002083 | 0 | 0 | 0 | 0 | 0 | 0 | 7.183 |
| OCBBF2002086 | 0 | 0 | 0 | 0 | 0 | 0 | 12.636 |
| OCBBF2002656 | 0 | 0 | 0 | 0 | 0 | 0 | 1.081 |
| OCBBF2002865 | 0 | 0 | 0 | 0 | 0 | 0 | 34.478 |
| OCBBF2003052 | 0 | 0 | 0 | 0 | 0 | 0 | 7.449 |
| OCBBF2003518 | 0 | 0 | 0 | 0 | 0 | 0 | 0.674 |
| OCBBF2003819 | 0 | 0 | 0 | 0 | 0 | 0 | 13.773 |
| OCBBF2003925 | 0 | 0 | 0 | 0 | 0 | 0 | 0.545 |
| OCBBF2004273 | 3.867 | 19.909 | 6.305 | 0 | 0 | 0 | 0.586 |
| OCBBF2004757 | 0 | 0 | 0 | 0 | 0 | 0 | 1.620 |
| OCBBF2005476 | 10.083 | 0 | 0 | 0 | 0 | 0 | 0 |
| OCBBF2005956 | 0 | 0 | 0 | 0 | 0 | 0 | 3.711 |
| OCBBF2006005 | 0 | 0 | 0 | 0 | 0 | 0 | 9.349 |
| OCBBF2006151 | 0 | 0 | 0 | 0 | 0 | 0 | 3.769 |
| OCBBF2006987 | 0 | 0 | 0 | 0 | 0 | 0 | 8.449 |
| OCBBF2007028 | 0 | 0 | 0 | 0 | 0 | 0 | 0.903 |
| OCBBF2007224 | 20.365 | 0 | 0 | 0 | 0 | 0 | 6.172 |
| OCBBF2007354 | 2.167 | 0 | 0 | 0 | 0 | 0 | 1.313 |
| OCBBF2007415 | 0 | 0 | 0 | 0 | 0 | 0 | 1.047 |
| OCBBF2007829 | 0 | 0 | 3.862 | 0 | 11.641 | 0 | 0 |
| OCBBF2007892 | 0 | 0 | 0 | 0 | 0 | 0 | 2.785 |
| OCBBF2008041 | 0 | 0 | 0 | 0 | 0 | 0 | 4.099 |
| OCBBF2008144 | 6.220 | 0 | 0 | 0 | 0 | 0 | 1.885 |
| OCBBF2008770 | 0 | 0 | 0 | 0 | 0 | 0 | 8.741 |
| OCBBF2009242 | 0 | 0 | 0 | 0 | 0 | 0 | 3.702 |
| OCBBF2009424 | 0 | 0 | 0 | 0 | 0 | 0 | 9.487 |
| OCBBF2009772 | 0 | 0 | 0 | 0 | 0 | 0 | 0.770 |
| OCBBF2009836 | 0 | 0 | 0 | 0 | 0 | 0 | 2.678 |
| OCBBF2010819 | 0 | 0 | 0 | 0 | 0 | 0 | 8.199 |
| OCBBF2010830 | 0 | 0 | 0 | 0 | 0 | 6.020 | 0.575 |
| OCBBF2011021 | 0 | 0 | 0 | 0 | 0 | 0 | 1.890 |
| OCBBF2011536 | 0 | 0 | 0 | 0 | 0 | 0 | 1.304 |
| OCBBF2011759 | 2.016 | 0 | 0 | 0 | 0 | 0 | 3.054 |
| OCBBF2012001 | 0 | 0 | 0 | 0 | 0 | 0 | 16.277 |
| OCBBF2012553 | 0 | 0 | 0 | 0 | 0 | 0 | 0.767 |
| OCBBF2012821 | 5.397 | 0 | 17.601 | 0 | 0 | 0 | 1.636 |
| OCBBF2014292 | 2.234 | 0 | 0 | 0 | 0 | 0 | 0 |
| OCBBF2014541 | 0 | 0 | 0 | 0 | 0 | 0 | 2.563 |
| OCBBF2014873 | 0 | 0 | 0 | 0 | 0 | 0 | 27.813 |
| OCBBF2015334 | 0 | 0 | 0 | 0 | 0 | 0 | 34.478 |
| OCBBF2015797 | 0 | 0 | 0 | 0 | 0 | 0 | 34.478 |
| OCBBF2016928 | 0 | 0 | 0 | 0 | 0 | 0 | 1.790 |
| OCBBF2017398 | 2.774 | 0 | 0 | 0 | 0 | 0 | 0.841 |
| OCBBF2017458 | 0 | 0 | 0 | 0 | 0 | 0 | 3.639 |
| OCBBF2017754 | 0 | 0 | 0 | 0 | 0 | 0 | 4.909 |
| OCBBF2017791 | 0 | 0 | 0 | 0 | 0 | 0 | 1.675 |
| OCBBF2017815 | 0 | 0 | 0 | 0 | 0 | 0 | 34.478 |
| OCBBF2017893 | 7.482 | 0 | 12.202 | 0 | 0 | 0 | 0 |
| OCBBF2017920 | 0 | 0 | 0 | 0 | 0 | 0 | 8.417 |
| OCBBF2018563 | 0 | 0 | 0 | 0 | 0 | 0 | 26.887 |
| OCBBF2018618 | 0 | 0 | 0 | 0 | 0 | 0 | 10.601 |
| OCBBF2018707 | 0 | 0 | 0 | 0 | 0 | 0 | 1.470 |
| OCBBF2018827 | 10.583 | 0 | 0 | 0 | 0 | 0 | 0 |
| OCBBF2018873 | 0 | 0 | 9.748 | 0 | 0 | 0 | 1.812 |
| OCBBF2018956 | 0 | 0 | 0 | 0 | 0 | 0 | 1.106 |
| OCBBF2020048 | 0 | 0 | 0 | 0 | 0 | 0 | 34.478 |
| OCBBF2021020 | 0 | 0 | 18.756 | 0 | 0 | 0 | 0 |
| OCBBF2024850 | 4.793 | 0 | 0 | 0 | 0 | 0 | 7.263 |
| OCBBF3000296 | 0 | 0 | 0 | 0 | 0 | 0 | 12.384 |
| OCBBF3000323 | 7.173 | 0 | 0 | 0 | 0 | 0 | 0 |
| OCBBF3009244 | 0 | 0 | 0 | 0 | 0 | 0 | 22.513 |
| PEBLM1000066 | 0 | 0 | 0 | 0 | 0 | 32.424 | 0 |
| PEBLM1000144 | 25.871 | 0 | 0 | 0 | 0 | 0 | 0 |
| PEBLM2000030 | 0 | 0 | 0 | 0 | 0 | 0 | 28.870 |
| PEBLM2000222 | 1.253 | 0 | 0 | 0 | 0 | 0 | 0 |
| PEBLM2000267 | 0 | 0 | 0 | 0 | 0 | 0 | 2.757 |
| PEBLM2000308 | 0 | 0 | 0 | 0 | 0 | 0 | 2.592 |
| PEBLM2000326 | 0 | 0 | 0 | 0 | 0 | 0 | 7.097 |
| PEBLM2000338 | 20.527 | 0 | 0 | 0 | 0 | 0 | 0 |
| PEBLM2001072 | 7.823 | 0 | 0 | 0 | 0 | 0 | 0 |
| PEBLM2001803 | 22.296 | 0 | 0 | 0 | 0 | 0 | 0 |
| PEBLM2002749 | 0 | 0 | 48.344 | 0 | 0 | 0 | 0 |
| PEBLM2003914 | 0 | 0 | 0 | 0 | 0 | 0 | 5.556 |
| PEBLM2004452 | 0 | 0 | 0 | 0 | 0 | 0 | 5.293 |
| PEBLM2004666 | 0 | 0 | 26.137 | 0 | 0 | 0 | 2.429 |
| PEBLM2004733 | 0 | 0 | 0 | 0 | 0 | 0 | 6.247 |
| PEBLM2007774 | 0 | 0 | 2.119 | 0 | 0 | 0 | 0.591 |
| PEBLM2008826 | 20.003 | 0 | 0 | 0 | 0 | 0 | 0 |
| PEBLM2008861 | 0 | 0 | 0 | 50.799 | 0 | 0 | 0 |
| PERIC2003794 | 0 | 0 | 0 | 0 | 0 | 0 | 8.846 |
| PERIC2007068 | 0 | 0 | 0 | 0 | 0 | 0 | 6.052 |
| PLACE5000013 | 10.271 | 0 | 0 | 0 | 0 | 0 | 10.376 |
| PLACE5000067 | 0 | 0 | 0 | 0 | 0 | 0 | 26.863 |
| PLACE5000068 | 0 | 0 | 0 | 0 | 0 | 0 | 4.105 |
| PLACE5000113 | 3.711 | 0 | 0 | 0 | 0 | 0 | 8.997 |
| PLACE5000159 | 0 | 0 | 0 | 0 | 0 | 0 | 1.486 |
| PLACE5000170 | 0 | 0 | 0 | 0 | 0 | 0 | 4.545 |
| PLACE6000145 | 0 | 0 | 0 | 0 | 21.972 | 0 | 0 |
| PLACE6000263 | 0 | 0 | 0 | 0 | 0 | 0 | 0.994 |
| PLACE6000379 | 0.762 | 0 | 0 | 0 | 3.745 | 2.417 | 0.808 |
| PLACE6000414 | 11.055 | 0 | 0 | 0 | 0 | 0 | 1.675 |
| PLACE6000463 | 0 | 0 | 0 | 0 | 0 | 0 | 5.100 |
| PLACE6001064 | 2.475 | 0 | 4.036 | 0 | 0 | 3.925 | 2.250 |
| PLACE6001281 | 0 | 0 | 0 | 0 | 0 | 0 | 2.250 |
| PLACE6002084 | 0.797 | 0 | 2.598 | 0 | 0 | 0 | 0.241 |
| PLACE6002102 | 3.046 | 0 | 0 | 0 | 0 | 0 | 2.770 |
| PLACE6002960 | 0 | 0 | 0 | 0 | 0 | 0 | 1.451 |
| PLACE6003038 | 0 | 0 | 0 | 0 | 0 | 0 | 2.359 |
| PLACE6003077 | 54.790 | 0 | 0 | 0 | 0 | 0 | 0 |
| PLACE6003383 | 7.723 | 0 | 0 | 0 | 0 | 0 | 0 |
| PLACE6003393 | 16.378 | 0 | 0 | 0 | 0 | 0 | 0 |
| PLACE6004312 | 0 | 0 | 0 | 0 | 0 | 0 | 0.394 |
| PLACE6004397 | 0 | 0 | 0 | 0 | 0 | 0 | 1.201 |
| PLACE6004578 | 2.674 | 0 | 0 | 0 | 0 | 0 | 1.621 |
| PLACE6005231 | 0 | 0 | 0 | 0 | 0 | 0 | 14.439 |
| PLACE6005482 | 0 | 0 | 0 | 0 | 0 | 0 | 3.457 |
| PLACE6006418 | 5.979 | 0 | 0 | 0 | 0 | 18.968 | 0 |
| PLACE6006549 | 0 | 0 | 22.469 | 0 | 0 | 0 | 0 |
| PLACE6008775 | 0 | 0 | 0 | 0 | 0 | 12.610 | 0 |
| PLACE6008793 | 0 | 0 | 0 | 0 | 0 | 0 | 1.036 |
| PLACE6010077 | 0 | 19.338 | 6.125 | 0 | 0 | 0 | 0 |
| PLACE6010484 | 0 | 0 | 21.747 | 0 | 0 | 0 | 1.010 |
| PLACE6011057 | 0 | 0 | 0 | 0 | 0 | 0 | 5.893 |
| PLACE6011334 | 0 | 0 | 0 | 0 | 0 | 0 | 41.126 |
| PLACE6012028 | 0 | 0 | 0 | 0 | 0 | 0 | 4.103 |
| PLACE6013222 | 0 | 0 | 0 | 0 | 0 | 0 | 9.663 |
| PLACE6015051 | 0 | 0 | 0 | 0 | 0 | 0 | 3.013 |
| PLACE6015513 | 54.790 | 0 | 0 | 0 | 0 | 0 | 0 |
| PLACE6016383 | 13.944 | 0 | 0 | 0 | 0 | 0 | 0 |
| PLACE6017788 | 0 | 0 | 0 | 0 | 0 | 0 | 26.863 |
| PLACE6017791 | 0 | 0 | 0 | 0 | 0 | 0 | 7.180 |
| PLACE6018031 | 0 | 0 | 0 | 0 | 0 | 0 | 4.395 |
| PLACE6018287 | 0 | 0 | 0 | 0 | 0 | 0 | 15.516 |
| PLACE6019195 | 0 | 0 | 0 | 0 | 0 | 0 | 5.547 |
| PLACE7001022 | 0 | 0 | 0 | 0 | 0 | 0 | 18.395 |
| PLACE7004103 | 2.789 | 0 | 0 | 0 | 0 | 0 | 9.299 |
| PROST1000097 | 0 | 0 | 0 | 0 | 12.466 | 4.023 | 0.769 |
| PROST1000144 | 0 | 0 | 0 | 0 | 0 | 0 | 4.099 |
| PROST1000199 | 0 | 0 | 0 | 0 | 0 | 0 | 0.478 |
| PROST1000220 | 0 | 0 | 0 | 0 | 0 | 9.290 | 0 |
| PROST1000226 | 3.859 | 0 | 0 | 0 | 0 | 0 | 0 |
| PROST1000564 | 0 | 0 | 0 | 0 | 0 | 0 | 0.879 |
| PROST2000079 | 0 | 0 | 0 | 0 | 0 | 0 | 1.957 |
| PROST2000138 | 0.374 | 1.925 | 0 | 0 | 0 | 1.186 | 0.567 |
| PROST2000284 | 0 | 0 | 24.634 | 0 | 0 | 0 | 2.289 |
| PROST2000325 | 0 | 0 | 0 | 0 | 0 | 0 | 4.627 |
| PROST2000567 | 0 | 0 | 0 | 0 | 0 | 0 | 18.726 |
| PROST2001180 | 0 | 0 | 0 | 0 | 0 | 0 | 1.623 |
| PROST2001521 | 0 | 0 | 0 | 0 | 25.760 | 0 | 0 |
| PROST2001552 | 0 | 0 | 85.728 | 0 | 0 | 0 | 0 |
| PROST2001883 | 0 | 19.838 | 0 | 0 | 0 | 0 | 0 |
| PROST2001997 | 0 | 0 | 0 | 0 | 0 | 0 | 7.622 |
| PROST2003232 | 2.943 | 0 | 0 | 0 | 0 | 0 | 0.892 |
| PROST2003338 | 0 | 0 | 7.874 | 0 | 0 | 0 | 0 |
| PROST2004095 | 0 | 0 | 0 | 0 | 0 | 3.202 | 0.612 |
| PROST2004258 | 0 | 0 | 0 | 0 | 0 | 0 | 2.494 |
| PROST2004270 | 2.537 | 0 | 0 | 0 | 0 | 0 | 1.538 |
| PROST2004416 | 1.467 | 0 | 4.783 | 0 | 0 | 0 | 0.444 |
| PROST2004739 | 0 | 0 | 0 | 0 | 0 | 0 | 19.443 |
| PROST2005604 | 6.709 | 0 | 0 | 0 | 0 | 0 | 0 |
| PROST2006060 | 14.401 | 0 | 0 | 0 | 0 | 0 | 0 |
| PROST2006282 | 0 | 0 | 0 | 0 | 0 | 0 | 5.807 |
| PROST2006343 | 0 | 0 | 31.103 | 0 | 0 | 0 | 0 |
| PROST2006737 | 0 | 0 | 0 | 0 | 0 | 16.583 | 0 |
| PROST2007317 | 0 | 0 | 0 | 0 | 0 | 0 | 15.564 |
| PROST2007612 | 0 | 0 | 0 | 0 | 0 | 21.849 | 0 |
| PROST2007871 | 0 | 0 | 0 | 0 | 0 | 0 | 7.210 |
| PROST2008489 | 2.794 | 0 | 0 | 0 | 0 | 0 | 0 |
| PROST2008770 | 0 | 0 | 0 | 0 | 0 | 0 | 1.020 |
| PROST2010579 | 0 | 0 | 0 | 0 | 0 | 0 | 12.287 |
| PROST2011105 | 0 | 0 | 0 | 0 | 0 | 0 | 11.632 |
| PROST2011577 | 0 | 0 | 0 | 43.435 | 0 | 0 | 0 |
| PROST2012007 | 0 | 0 | 0 | 23.952 | 0 | 0 | 0.742 |
| PROST2012448 | 0 | 0 | 0 | 0 | 0 | 0 | 10.667 |
| PROST2012745 | 8.341 | 0 | 0 | 0 | 0 | 0 | 0 |
| PROST2013121 | 0 | 0 | 0 | 0 | 0 | 0 | 14.062 |
| PROST2015137 | 0 | 0 | 0 | 0 | 0 | 0 | 12.011 |
| PROST2017203 | 2.801 | 0 | 0 | 0 | 0 | 0 | 0.849 |
| PROST2019164 | 15.906 | 0 | 0 | 0 | 0 | 0 | 0 |
| PROST2019253 | 3.624 | 0 | 0 | 0 | 0 | 0 | 0 |
| PUAEN1000057 | 4.428 | 0 | 0 | 0 | 0 | 0 | 0 |
| PUAEN1000065 | 0 | 0 | 4.712 | 0 | 0 | 0 | 0 |
| PUAEN1000161 | 0 | 0 | 0 | 0 | 0 | 0 | 7.541 |
| PUAEN1000164 | 1.717 | 0 | 0 | 0 | 0 | 0 | 0.260 |
| PUAEN1000239 | 0 | 0 | 0 | 0 | 0 | 0 | 2.026 |
| PUAEN1000322 | 0 | 0 | 0 | 0 | 0 | 8.747 | 0 |
| PUAEN2000152 | 0 | 0 | 0 | 0 | 0 | 0 | 7.759 |
| PUAEN2000247 | 0 | 0 | 0 | 0 | 0 | 0 | 18.058 |
| PUAEN2000374 | 0 | 0 | 0 | 0 | 0 | 0 | 0.628 |
| PUAEN2000535 | 0 | 0 | 0 | 0 | 0 | 0 | 1.577 |
| PUAEN2001586 | 0 | 0 | 0 | 0 | 0 | 0 | 1.867 |
| PUAEN2005502 | 0 | 0 | 0 | 0 | 0 | 0 | 20.989 |
| PUAEN2007044 | 0 | 0 | 0 | 0 | 0 | 0 | 7.496 |
| PUAEN2008985 | 9.570 | 0 | 0 | 0 | 0 | 30.358 | 17.402 |
| RECTM1000051 | 0 | 0 | 0 | 0 | 0 | 15.792 | 0 |
| SALGL1000047 | 0 | 0 | 0 | 0 | 0 | 0 | 0.194 |
| SALGL1000107 | 0 | 0 | 0 | 0 | 0 | 0 | 0.404 |
| SALGL1000157 | 0.561 | 0 | 0 | 0 | 5.514 | 0 | 0 |
| SKMUS1000014 | 0 | 0 | 0 | 0 | 0 | 0 | 4.809 |
| SKMUS1000022 | 0 | 9.245 | 0 | 0 | 0 | 0 | 0 |
| SKMUS1000084 | 0 | 0 | 0 | 0 | 32.651 | 0 | 2.013 |
| SKMUS1000138 | 0 | 0 | 0 | 0 | 0 | 0 | 0.126 |
| SKMUS1000176 | 0 | 0 | 0 | 0 | 18.291 | 0 | 0 |
| SKMUS1000186 | 0.866 | 0 | 0 | 0 | 0 | 0 | 0.787 |
| SKMUS2000243 | 21.758 | 0 | 0 | 0 | 0 | 0 | 0 |
| SKMUS2000361 | 0 | 0 | 0 | 0 | 0 | 0 | 1.398 |
| SKMUS2000484 | 3.787 | 0 | 0 | 0 | 0 | 0 | 0 |
| SKMUS2000679 | 0 | 0 | 0 | 0 | 0 | 0 | 0.438 |
| SKMUS2000902 | 11.665 | 0 | 0 | 0 | 0 | 0 | 0 |
| SKMUS2000931 | 0 | 12.086 | 0 | 0 | 0 | 0 | 0.711 |
| SKMUS2000933 | 13.382 | 0 | 0 | 0 | 0 | 0 | 0 |
| SKMUS2000954 | 7.332 | 0 | 0 | 0 | 0 | 0 | 0 |
| SKMUS2000961 | 0 | 0 | 46.311 | 0 | 0 | 0 | 0 |
| SKMUS2001147 | 0 | 0 | 0 | 0 | 0 | 0 | 1.812 |
| SKMUS2001323 | 0 | 0 | 0 | 37.072 | 0 | 0 | 0 |
| SKMUS2001454 | 0.728 | 0 | 2.374 | 0 | 0 | 0 | 1.324 |
| SKMUS2001528 | 0 | 0 | 8.830 | 0 | 0 | 8.588 | 1.641 |
| SKMUS2001634 | 2.824 | 0 | 0 | 0 | 0 | 0 | 4.279 |
| SKMUS2003194 | 0 | 0 | 0 | 0 | 0 | 0 | 8.489 |
| SKMUS2004044 | 0 | 0 | 0 | 0 | 0 | 0 | 8.489 |
| SKMUS2004908 | 0 | 0 | 0 | 0 | 0 | 0 | 2.147 |
| SKNMC1000137 | 0 | 0 | 0 | 0 | 0 | 0 | 1.386 |
| SKNMC1000159 | 0 | 0 | 0 | 0 | 0 | 0 | 6.903 |
| SKNMC1000168 | 0 | 0 | 0 | 0 | 0 | 0 | 1.095 |
| SKNMC2000065 | 4.657 | 0 | 0 | 0 | 0 | 0 | 2.117 |
| SKNMC2000097 | 0 | 0 | 0 | 0 | 0 | 0 | 0.291 |
| SKNMC2000305 | 0 | 0 | 0 | 0 | 0 | 0 | 4.605 |
| SKNMC2000322 | 0 | 0 | 0 | 0 | 0 | 0 | 3.693 |
| SKNMC2000635 | 0.774 | 3.984 | 0 | 0 | 0 | 0 | 0.117 |
| SKNMC2000649 | 1.968 | 0 | 0 | 0 | 0 | 0 | 0 |
| SKNMC2000877 | 0 | 0 | 0 | 0 | 0 | 0 | 2.324 |
| SKNMC2001324 | 0 | 0 | 0 | 0 | 0 | 0 | 4.221 |
| SKNMC2001623 | 21.672 | 0 | 0 | 0 | 0 | 0 | 0 |
| SKNSH1000101 | 0 | 0 | 0 | 0 | 13.619 | 4.395 | 0 |
| SKNSH2000151 | 0 | 0 | 0 | 0 | 0 | 0 | 3.556 |
| SKNSH2000347 | 0 | 0 | 0 | 0 | 0 | 0 | 1.467 |
| SKNSH2000819 | 0 | 0 | 0 | 0 | 0 | 0 | 4.881 |
| SKNSH2003633 | 0 | 0 | 47.185 | 0 | 0 | 0 | 0 |
| SKNSH2005240 | 0 | 0 | 0 | 0 | 0 | 0 | 8.693 |
| SMINT1000016 | 0 | 0 | 0 | 0 | 0 | 0 | 1.604 |
| SMINT1000057 | 37.935 | 0 | 0 | 0 | 0 | 0 | 0 |
| SMINT1000117 | 0 | 29.214 | 0 | 0 | 0 | 0 | 4.299 |
| SMINT2000018 | 0 | 0 | 29.616 | 0 | 0 | 0 | 0 |
| SMINT2000441 | 0 | 0 | 9.526 | 0 | 0 | 6.176 | 1.180 |
| SMINT2000454 | 0 | 0 | 0 | 0 | 0 | 0 | 11.684 |
| SMINT2000545 | 10.226 | 0 | 0 | 0 | 0 | 0 | 0 |
| SMINT2000629 | 0 | 0 | 0 | 0 | 0 | 0 | 1.755 |
| SMINT2000659 | 0 | 0 | 0 | 0 | 0 | 1.901 | 0.182 |
| SMINT2000694 | 2.564 | 0 | 0 | 0 | 25.200 | 0 | 0 |
| SMINT2000933 | 6.594 | 0 | 0 | 0 | 0 | 0 | 0 |
| SMINT2001812 | 0 | 0 | 0 | 0 | 0 | 0 | 15.682 |
| SMINT2001818 | 2.022 | 0 | 0 | 0 | 0 | 0 | 0 |
| SMINT2002314 | 0 | 0 | 8.514 | 0 | 0 | 0 | 0 |
| SMINT2002689 | 2.535 | 0 | 0 | 0 | 0 | 0 | 0.768 |
| SMINT2002882 | 0 | 0 | 0 | 0 | 0 | 0 | 1.898 |
| SMINT2003074 | 0 | 0 | 0 | 28.929 | 0 | 0 | 0 |
| SMINT2003169 | 0 | 0 | 0 | 0 | 0 | 0 | 0.307 |
| SMINT2003905 | 0 | 0 | 0 | 0 | 0 | 5.297 | 2.024 |
| SMINT2004299 | 0 | 0 | 0 | 0 | 0 | 61.748 | 0 |
| SMINT2004589 | 0 | 0 | 28.193 | 0 | 0 | 0 | 0 |
| SMINT2004872 | 0 | 0 | 29.754 | 0 | 0 | 0 | 0 |
| SMINT2005368 | 2.533 | 26.084 | 0 | 0 | 0 | 8.035 | 0 |
| SMINT2006801 | 0 | 0 | 0 | 0 | 0 | 0 | 10.528 |
| SMINT2007729 | 0 | 0 | 0 | 0 | 0 | 39.345 | 0 |
| SMINT2008921 | 0 | 24.644 | 0 | 0 | 0 | 7.591 | 0.725 |
| SMINT2008960 | 5.902 | 0 | 4.813 | 0 | 0 | 0 | 1.342 |
| SMINT2009292 | 0 | 0 | 0 | 0 | 0 | 24.268 | 4.637 |
| SMINT2010639 | 0 | 0 | 0 | 0 | 0 | 0 | 1.771 |
| SMINT2010901 | 0 | 0 | 0 | 0 | 0 | 0 | 6.887 |
| SMINT2012122 | 7.540 | 0 | 0 | 0 | 0 | 0 | 0 |
| SMINT2013031 | 0 | 0 | 0 | 0 | 0 | 0 | 6.086 |
| SMINT2013032 | 25.797 | 0 | 0 | 0 | 0 | 0 | 0 |
| SMINT2014480 | 0 | 0 | 22.352 | 0 | 0 | 0 | 0 |
| SMINT2015294 | 17.171 | 0 | 0 | 0 | 0 | 0 | 0 |
| SMINT2016412 | 0 | 0 | 0 | 0 | 80.022 | 0 | 0 |
| SPLEN1000106 | 5.020 | 0 | 0 | 0 | 0 | 0 | 6.086 |
| SPLEN1000116 | 0 | 0 | 3.482 | 0 | 0 | 0 | 0 |
| SPLEN1000165 | 0 | 0 | 0 | 0 | 0 | 34.144 | 3.262 |
| SPLEN1000166 | 0 | 0 | 0 | 0 | 0 | 0 | 1.478 |
| SPLEN2000255 | 0 | 0 | 0 | 0 | 0 | 0 | 2.008 |
| SPLEN2000267 | 0 | 0 | 0 | 0 | 0 | 0 | 3.816 |
| SPLEN2000348 | 0 | 0 | 0 | 0 | 0 | 0 | 1.829 |
| SPLEN2000505 | 3.636 | 9.360 | 0 | 0 | 0 | 0 | 0.275 |
| SPLEN2000516 | 25.781 | 0 | 0 | 0 | 0 | 0 | 7.814 |
| SPLEN2000839 | 0 | 0 | 0 | 0 | 0 | 0 | 5.966 |
| SPLEN2000874 | 1.051 | 0 | 0 | 0 | 0 | 0 | 0 |
| SPLEN2000882 | 7.578 | 0 | 0 | 0 | 0 | 12.019 | 0 |
| SPLEN2001141 | 34.624 | 0 | 0 | 0 | 0 | 0 | 1.235 |
| SPLEN2001599 | 0 | 0 | 50.196 | 0 | 0 | 0 | 0 |
| SPLEN2001761 | 22.950 | 0 | 0 | 0 | 0 | 0 | 0 |
| SPLEN2002133 | 0 | 0 | 0 | 0 | 0 | 0 | 27.313 |
| SPLEN2002335 | 0 | 0 | 0 | 0 | 0 | 0 | 0.662 |
| SPLEN2004220 | 0 | 0 | 0 | 0 | 0 | 0 | 1.880 |
| SPLEN2004555 | 0 | 0 | 0 | 0 | 0 | 0 | 4.870 |
| SPLEN2004773 | 0 | 0 | 5.007 | 0 | 0 | 0 | 0 |
| SPLEN2004880 | 0 | 0 | 0 | 0 | 0 | 0 | 0.193 |
| SPLEN2004984 | 11.330 | 0 | 0 | 0 | 0 | 0 | 0 |
| SPLEN2005429 | 0 | 0 | 0 | 0 | 0 | 0 | 2.395 |
| SPLEN2005818 | 24.041 | 0 | 0 | 0 | 0 | 0 | 0 |
| SPLEN2005869 | 13.851 | 0 | 0 | 0 | 0 | 0 | 0 |
| SPLEN2006122 | 0 | 18.706 | 0 | 0 | 0 | 0 | 0.551 |
| SPLEN2006133 | 2.046 | 0 | 0 | 0 | 0 | 0 | 0 |
| SPLEN2006232 | 18.561 | 0 | 0 | 0 | 0 | 0 | 0 |
| SPLEN2006305 | 1.362 | 0 | 4.444 | 0 | 0 | 0 | 1.652 |
| SPLEN2006374 | 0 | 0 | 0 | 0 | 0 | 0 | 0.994 |
| SPLEN2007498 | 0 | 0 | 0 | 0 | 0 | 0 | 3.421 |
| SPLEN2007739 | 0 | 0 | 0 | 0 | 0 | 0 | 3.969 |
| SPLEN2007750 | 10.435 | 0 | 0 | 0 | 0 | 0 | 0 |
| SPLEN2008460 | 2.826 | 0 | 0 | 0 | 0 | 0 | 0 |
| SPLEN2008591 | 0 | 0 | 0 | 0 | 0 | 0 | 27.313 |
| SPLEN2009081 | 0 | 0 | 0 | 0 | 0 | 44.282 | 0 |
| SPLEN2009555 | 18.320 | 0 | 0 | 0 | 0 | 0 | 0 |
| SPLEN2009970 | 10.999 | 0 | 0 | 0 | 0 | 0 | 6.667 |
| SPLEN2010195 | 4.184 | 0 | 2.729 | 0 | 0 | 0 | 0.761 |
| SPLEN2010986 | 0 | 0 | 0 | 0 | 0 | 0 | 13.164 |
| SPLEN2011419 | 5.154 | 0 | 0 | 0 | 0 | 0 | 1.562 |
| SPLEN2012571 | 6.857 | 0 | 0 | 0 | 0 | 0 | 0 |
| SPLEN2012800 | 0.737 | 0 | 2.403 | 0 | 0 | 2.337 | 0 |
| SPLEN2014080 | 0 | 0 | 0 | 0 | 0 | 0 | 3.097 |
| SPLEN2014572 | 13.423 | 0 | 0 | 0 | 0 | 0 | 0 |
| SPLEN2014911 | 0 | 0 | 0 | 0 | 0 | 7.218 | 0 |
| SPLEN2015310 | 0 | 0 | 0 | 0 | 0 | 3.922 | 0.375 |
| SPLEN2015788 | 20.881 | 0 | 0 | 0 | 0 | 0 | 0 |
| SPLEN2016268 | 0.932 | 0 | 0 | 9.118 | 0 | 0 | 1.130 |
| SPLEN2016421 | 4.468 | 0 | 14.573 | 0 | 0 | 14.174 | 1.354 |
| SPLEN2016554 | 0 | 0 | 0 | 0 | 0 | 0 | 12.183 |
| SPLEN2016972 | 0 | 0 | 0 | 0 | 0 | 0 | 2.651 |
| SPLEN2017043 | 0 | 0 | 73.215 | 0 | 0 | 0 | 0 |
| SPLEN2017121 | 0 | 0 | 54.539 | 0 | 0 | 0 | 0 |
| SPLEN2017318 | 0 | 0 | 0 | 0 | 0 | 0 | 3.317 |
| SPLEN2017351 | 6.487 | 0 | 0 | 0 | 0 | 0 | 7.865 |
| SPLEN2019169 | 0 | 0 | 0 | 0 | 0 | 0 | 7.401 |
| SPLEN2019405 | 0 | 0 | 0 | 0 | 0 | 0 | 5.617 |
| SPLEN2021194 | 7.276 | 0 | 0 | 0 | 0 | 0 | 0 |
| SPLEN2022522 | 12.230 | 0 | 0 | 0 | 0 | 0 | 0.927 |
| SPLEN2023160 | 0 | 0 | 0 | 0 | 0 | 0 | 25.698 |
| SPLEN2023733 | 0 | 0 | 0 | 0 | 0 | 0 | 13.161 |
| SPLEN2026411 | 0 | 0 | 80.173 | 0 | 0 | 0 | 0 |
| SPLEN2027113 | 0 | 0 | 0 | 0 | 0 | 0 | 1.070 |
| SPLEN2028466 | 0.892 | 0 | 2.910 | 0 | 0 | 0 | 0 |
| SPLEN2028914 | 6.023 | 0 | 0 | 0 | 0 | 0 | 0 |
| SPLEN2031547 | 3.396 | 0 | 11.076 | 0 | 0 | 0 | 1.029 |
| SPLEN2036076 | 10.630 | 0 | 11.557 | 0 | 0 | 0 | 0 |
| SPLEN2038345 | 0 | 0 | 0 | 0 | 0 | 0 | 19.221 |
| SPLEN2038834 | 0 | 0 | 32.588 | 0 | 0 | 0 | 0 |
| SPLEN2039379 | 0 | 0 | 0 | 0 | 0 | 0 | 7.573 |
| SPLEN2042535 | 0 | 0 | 0 | 0 | 0 | 0 | 4.654 |
| STOMA1000189 | 0 | 0 | 22.002 | 0 | 0 | 0 | 0 |
| STOMA2000121 | 0 | 0 | 0 | 0 | 0 | 8.338 | 0 |
| STOMA2000257 | 4.257 | 0 | 0 | 41.658 | 0 | 0 | 2.581 |
| STOMA2000289 | 0 | 0 | 0 | 0 | 0 | 0 | 3.562 |
| STOMA2000395 | 0 | 0 | 0 | 0 | 0 | 14.605 | 0 |
| STOMA2000396 | 0 | 0 | 0 | 0 | 0 | 0 | 3.369 |
| STOMA2001389 | 0 | 0 | 0 | 0 | 0 | 39.515 | 0 |
| STOMA2002052 | 0 | 0 | 0 | 0 | 0 | 0 | 7.271 |
| STOMA2002166 | 0 | 0 | 0 | 0 | 0 | 0 | 0.765 |
| STOMA2002828 | 0 | 0 | 0 | 0 | 0 | 37.497 | 0 |
| STOMA2003444 | 0 | 0 | 0 | 0 | 0 | 0 | 8.764 |
| STOMA2004893 | 0 | 0 | 50.829 | 0 | 0 | 0 | 0 |
| STOMA2005782 | 0 | 0 | 0 | 0 | 0 | 0 | 8.764 |
| STOMA2006229 | 12.967 | 0 | 0 | 0 | 0 | 0 | 0 |
| STOMA2008638 | 0 | 0 | 0 | 0 | 0 | 50.134 | 0 |
| SYNOV1000045 | 15.151 | 0 | 0 | 0 | 0 | 0 | 0 |
| SYNOV1000049 | 47.227 | 0 | 0 | 0 | 0 | 0 | 0 |
| SYNOV1000118 | 45.640 | 0 | 0 | 0 | 0 | 0 | 0 |
| SYNOV1000124 | 1.200 | 0 | 3.913 | 0 | 0 | 3.806 | 0 |
| SYNOV1000128 | 31.662 | 0 | 0 | 0 | 0 | 0 | 0 |
| SYNOV1000164 | 8.294 | 0 | 0 | 0 | 0 | 0 | 0 |
| SYNOV1000190 | 100 | 0 | 0 | 0 | 0 | 0 | 0 |
| SYNOV1000256 | 100 | 0 | 0 | 0 | 0 | 0 | 0 |
| SYNOV1000374 | 42.798 | 0 | 0 | 0 | 0 | 0 | 12.971 |
| SYNOV2000152 | 100 | 0 | 0 | 0 | 0 | 0 | 0 |
| SYNOV2000171 | 100 | 0 | 0 | 0 | 0 | 0 | 0 |
| SYNOV2000173 | 100 | 0 | 0 | 0 | 0 | 0 | 0 |
| SYNOV2000177 | 100 | 0 | 0 | 0 | 0 | 0 | 0 |
| SYNOV2000178 | 100 | 0 | 0 | 0 | 0 | 0 | 0 |
| SYNOV2000193 | 100 | 0 | 0 | 0 | 0 | 0 | 0 |
| SYNOV2000251 | 100 | 0 | 0 | 0 | 0 | 0 | 0 |
| SYNOV2000279 | 100 | 0 | 0 | 0 | 0 | 0 | 0 |
| SYNOV2000291 | 67.888 | 0 | 0 | 0 | 0 | 0 | 0 |
| SYNOV2000297 | 100 | 0 | 0 | 0 | 0 | 0 | 0 |
| SYNOV2000397 | 51.386 | 0 | 0 | 0 | 0 | 0 | 0 |
| SYNOV2000426 | 100 | 0 | 0 | 0 | 0 | 0 | 0 |
| SYNOV2000601 | 100 | 0 | 0 | 0 | 0 | 0 | 0 |
| SYNOV2000700 | 100 | 0 | 0 | 0 | 0 | 0 | 0 |
| SYNOV2000820 | 75.240 | 0 | 0 | 0 | 0 | 0 | 0 |
| SYNOV2000824 | 100 | 0 | 0 | 0 | 0 | 0 | 0 |
| SYNOV2000923 | 100 | 0 | 0 | 0 | 0 | 0 | 0 |
| SYNOV2001014 | 100 | 0 | 0 | 0 | 0 | 0 | 0 |
| SYNOV2001033 | 1.293 | 13.317 | 4.218 | 0 | 0 | 8.204 | 0.392 |
| SYNOV2001035 | 100 | 0 | 0 | 0 | 0 | 0 | 0 |
| SYNOV2001088 | 100 | 0 | 0 | 0 | 0 | 0 | 0 |
| SYNOV2001111 | 100 | 0 | 0 | 0 | 0 | 0 | 0 |
| SYNOV2001144 | 3.051 | 0 | 0 | 0 | 0 | 9.677 | 0.925 |
| SYNOV2001212 | 23.502 | 0 | 0 | 0 | 0 | 0 | 0 |
| SYNOV2001239 | 0.677 | 0 | 0 | 0 | 0 | 0 | 0 |
| SYNOV2001251 | 100 | 0 | 0 | 0 | 0 | 0 | 0 |
| SYNOV2001262 | 4.500 | 0 | 0 | 0 | 0 | 0 | 1.364 |
| SYNOV2001300 | 100 | 0 | 0 | 0 | 0 | 0 | 0 |
| SYNOV2001356 | 100 | 0 | 0 | 0 | 0 | 0 | 0 |
| SYNOV2001374 | 3.950 | 0 | 0 | 0 | 0 | 0 | 0 |
| SYNOV2001390 | 100 | 0 | 0 | 0 | 0 | 0 | 0 |
| SYNOV2001451 | 100 | 0 | 0 | 0 | 0 | 0 | 0 |
| SYNOV2001457 | 100 | 0 | 0 | 0 | 0 | 0 | 0 |
| SYNOV2001468 | 19.684 | 0 | 0 | 0 | 0 | 0 | 0 |
| SYNOV2001581 | 11.360 | 0 | 0 | 0 | 55.833 | 0 | 0 |
| SYNOV2001648 | 7.012 | 0 | 7.623 | 0 | 22.975 | 0 | 0 |
| SYNOV2001660 | 100 | 0 | 0 | 0 | 0 | 0 | 0 |
| SYNOV2003326 | 100 | 0 | 0 | 0 | 0 | 0 | 0 |
| SYNOV2005216 | 21.061 | 0 | 0 | 0 | 0 | 66.809 | 0 |
| SYNOV2005448 | 100 | 0 | 0 | 0 | 0 | 0 | 0 |
| SYNOV2005541 | 30.878 | 0 | 0 | 0 | 0 | 0 | 0 |
| SYNOV2005673 | 100 | 0 | 0 | 0 | 0 | 0 | 0 |
| SYNOV2005817 | 100 | 0 | 0 | 0 | 0 | 0 | 0 |
| SYNOV2005962 | 100 | 0 | 0 | 0 | 0 | 0 | 0 |
| SYNOV2006430 | 100 | 0 | 0 | 0 | 0 | 0 | 0 |
| SYNOV2006620 | 66.043 | 0 | 0 | 0 | 0 | 0 | 0 |
| SYNOV2007758 | 42.201 | 0 | 0 | 0 | 0 | 0 | 0 |
| SYNOV2007965 | 100 | 0 | 0 | 0 | 0 | 0 | 0 |
| SYNOV2008765 | 3.113 | 0 | 0 | 0 | 0 | 0 | 0 |
| SYNOV2009172 | 1.122 | 0 | 0 | 0 | 0 | 3.560 | 0 |
| SYNOV2011233 | 100 | 0 | 0 | 0 | 0 | 0 | 0 |
| SYNOV2012326 | 100 | 0 | 0 | 0 | 0 | 0 | 0 |
| SYNOV2013365 | 100 | 0 | 0 | 0 | 0 | 0 | 0 |
| SYNOV2013637 | 100 | 0 | 0 | 0 | 0 | 0 | 0 |
| SYNOV2014157 | 76.742 | 0 | 0 | 0 | 0 | 0 | 23.258 |
| SYNOV2014364 | 100 | 0 | 0 | 0 | 0 | 0 | 0 |
| SYNOV2016124 | 100 | 0 | 0 | 0 | 0 | 0 | 0 |
| SYNOV2016837 | 100 | 0 | 0 | 0 | 0 | 0 | 0 |
| SYNOV2017055 | 36.415 | 0 | 0 | 0 | 0 | 0 | 0 |
| SYNOV2017179 | 100 | 0 | 0 | 0 | 0 | 0 | 0 |
| SYNOV2018921 | 100 | 0 | 0 | 0 | 0 | 0 | 0 |
| SYNOV2019280 | 100 | 0 | 0 | 0 | 0 | 0 | 0 |
| SYNOV2020085 | 100 | 0 | 0 | 0 | 0 | 0 | 0 |
| SYNOV2020463 | 9.451 | 0 | 0 | 0 | 0 | 0 | 0 |
| SYNOV2021320 | 100 | 0 | 0 | 0 | 0 | 0 | 0 |
| SYNOV2021953 | 61.927 | 0 | 0 | 0 | 0 | 0 | 31.281 |
| SYNOV3000184 | 100 | 0 | 0 | 0 | 0 | 0 | 0 |
| SYNOV3000231 | 100 | 0 | 0 | 0 | 0 | 0 | 0 |
| SYNOV3000302 | 100 | 0 | 0 | 0 | 0 | 0 | 0 |
| SYNOV3000345 | 100 | 0 | 0 | 0 | 0 | 0 | 0 |
| SYNOV4000249 | 51.386 | 0 | 0 | 0 | 0 | 0 | 0 |
| SYNOV4000472 | 100 | 0 | 0 | 0 | 0 | 0 | 0 |
| SYNOV4000598 | 64.392 | 0 | 0 | 0 | 0 | 0 | 0 |
| SYNOV4000706 | 100 | 0 | 0 | 0 | 0 | 0 | 0 |
| SYNOV4001153 | 5.003 | 0 | 0 | 0 | 0 | 0 | 0 |
| SYNOV4001224 | 64.392 | 0 | 0 | 0 | 0 | 0 | 0 |
| SYNOV4001326 | 65.818 | 0 | 0 | 0 | 0 | 0 | 0 |
| SYNOV4001342 | 100 | 0 | 0 | 0 | 0 | 0 | 0 |
| SYNOV4001395 | 78.340 | 0 | 0 | 0 | 0 | 0 | 0 |
| SYNOV4002001 | 100 | 0 | 0 | 0 | 0 | 0 | 0 |
| SYNOV4002392 | 100 | 0 | 0 | 0 | 0 | 0 | 0 |
| SYNOV4002875 | 100 | 0 | 0 | 0 | 0 | 0 | 0 |
| SYNOV4002883 | 51.635 | 0 | 0 | 0 | 0 | 0 | 0 |
| SYNOV4003174 | 100 | 0 | 0 | 0 | 0 | 0 | 0 |
| SYNOV4003322 | 100 | 0 | 0 | 0 | 0 | 0 | 0 |
| SYNOV4003681 | 100 | 0 | 0 | 0 | 0 | 0 | 0 |
| SYNOV4003981 | 100 | 0 | 0 | 0 | 0 | 0 | 0 |
| SYNOV4004184 | 100 | 0 | 0 | 0 | 0 | 0 | 0 |
| SYNOV4004741 | 100 | 0 | 0 | 0 | 0 | 0 | 0 |
| SYNOV4004823 | 49.051 | 0 | 0 | 0 | 0 | 0 | 0 |
| SYNOV4004914 | 100 | 0 | 0 | 0 | 0 | 0 | 0 |
| SYNOV4005256 | 76.742 | 0 | 0 | 0 | 0 | 0 | 23.258 |
| SYNOV4005570 | 100 | 0 | 0 | 0 | 0 | 0 | 0 |
| SYNOV4005739 | 100 | 0 | 0 | 0 | 0 | 0 | 0 |
| SYNOV4005889 | 100 | 0 | 0 | 0 | 0 | 0 | 0 |
| SYNOV4005981 | 100 | 0 | 0 | 0 | 0 | 0 | 0 |
| SYNOV4005989 | 44.519 | 0 | 0 | 0 | 0 | 0 | 0 |
| SYNOV4006256 | 100 | 0 | 0 | 0 | 0 | 0 | 0 |
| SYNOV4006327 | 33.336 | 0 | 0 | 0 | 0 | 0 | 0 |
| SYNOV4006542 | 87.954 | 0 | 0 | 0 | 0 | 0 | 0 |
| SYNOV4007012 | 72.118 | 0 | 0 | 0 | 0 | 0 | 0 |
| SYNOV4007215 | 100 | 0 | 0 | 0 | 0 | 0 | 0 |
| SYNOV4007360 | 100 | 0 | 0 | 0 | 0 | 0 | 0 |
| SYNOV4007430 | 68.323 | 0 | 0 | 0 | 0 | 0 | 0 |
| SYNOV4007521 | 100 | 0 | 0 | 0 | 0 | 0 | 0 |
| SYNOV4007553 | 72.118 | 0 | 0 | 0 | 0 | 0 | 0 |
| SYNOV4007671 | 1.581 | 0 | 0 | 0 | 0 | 0 | 1.917 |
| SYNOV4007711 | 100 | 0 | 0 | 0 | 0 | 0 | 0 |
| SYNOV4008245 | 19.926 | 0 | 0 | 0 | 0 | 0 | 0 |
| SYNOV4008336 | 100 | 0 | 0 | 0 | 0 | 0 | 0 |
| SYNOV4009129 | 16.744 | 0 | 0 | 0 | 0 | 0 | 0 |
| SYNOV4009139 | 100 | 0 | 0 | 0 | 0 | 0 | 0 |
| SYNOV4009298 | 63.453 | 0 | 0 | 0 | 0 | 0 | 0 |
| SYNOV4009575 | 10.334 | 0 | 0 | 0 | 0 | 0 | 0 |
| T1ESE2000116 | 0 | 96.382 | 0 | 0 | 0 | 0 | 0 |
| T1ESE2000609 | 0 | 50.485 | 0 | 0 | 0 | 0 | 0 |
| T1ESE2000904 | 0 | 51.278 | 0 | 0 | 0 | 0 | 0 |
| T1ESE2002665 | 0 | 92.582 | 0 | 0 | 0 | 0 | 0 |
| TBAES2000004 | 0 | 0 | 100 | 0 | 0 | 0 | 0 |
| TBAES2000059 | 0 | 0 | 84.801 | 0 | 0 | 0 | 0 |
| TBAES2000116 | 0 | 0 | 79.809 | 0 | 0 | 0 | 0 |
| TBAES2000255 | 0 | 0 | 100 | 0 | 0 | 0 | 0 |
| TBAES2000315 | 0 | 0 | 100 | 0 | 0 | 0 | 0 |
| TBAES2000352 | 0 | 0 | 50.050 | 0 | 0 | 0 | 0 |
| TBAES2000932 | 0 | 0 | 100 | 0 | 0 | 0 | 0 |
| TBAES2001171 | 0 | 0 | 100 | 0 | 0 | 0 | 0 |
| TBAES2001220 | 0 | 0 | 100 | 0 | 0 | 0 | 0 |
| TBAES2001229 | 0 | 0 | 100 | 0 | 0 | 0 | 0 |
| TBAES2001258 | 0 | 0 | 66.863 | 0 | 0 | 0 | 0 |
| TBAES2001296 | 0 | 0 | 79.254 | 0 | 0 | 0 | 0 |
| TBAES2001492 | 0 | 0 | 100 | 0 | 0 | 0 | 0 |
| TBAES2001553 | 6.344 | 0 | 20.692 | 62.076 | 0 | 0 | 0 |
| TBAES2001751 | 0 | 0 | 100 | 0 | 0 | 0 | 0 |
| TBAES2003435 | 0 | 0 | 100 | 0 | 0 | 0 | 0 |
| TBAES2003492 | 0 | 0 | 100 | 0 | 0 | 0 | 0 |
| TBAES2003550 | 0 | 0 | 100 | 0 | 0 | 0 | 0 |
| TBAES2003702 | 0 | 0 | 85.503 | 0 | 0 | 0 | 0 |
| TBAES2003917 | 0 | 0 | 100 | 0 | 0 | 0 | 0 |
| TBAES2004055 | 0 | 0 | 100 | 0 | 0 | 0 | 0 |
| TBAES2004105 | 0 | 0 | 100 | 0 | 0 | 0 | 0 |
| TBAES2004939 | 0 | 0 | 100 | 0 | 0 | 0 | 0 |
| TBAES2005157 | 0 | 0 | 100 | 0 | 0 | 0 | 0 |
| TBAES2005361 | 0 | 0 | 100 | 0 | 0 | 0 | 0 |
| TBAES2005543 | 0 | 0 | 100 | 0 | 0 | 0 | 0 |
| TBAES2006568 | 0 | 0 | 100 | 0 | 0 | 0 | 0 |
| TBAES2006881 | 0 | 0 | 100 | 0 | 0 | 0 | 0 |
| TBAES2007379 | 0 | 0 | 100 | 0 | 0 | 0 | 0 |
| TBAES2007428 | 0 | 0 | 37.783 | 0 | 0 | 0 | 0 |
| TBAES2007481 | 0 | 0 | 100 | 0 | 0 | 0 | 0 |
| TBAES2007504 | 0 | 0 | 100 | 0 | 0 | 0 | 0 |
| TBAES2007548 | 0 | 0 | 100 | 0 | 0 | 0 | 0 |
| TBAES2007697 | 0 | 0 | 100 | 0 | 0 | 0 | 0 |
| TBAES2007862 | 0 | 0 | 100 | 0 | 0 | 0 | 0 |
| TBAES2007964 | 0 | 0 | 100 | 0 | 0 | 0 | 0 |
| TBAES2008133 | 0 | 0 | 100 | 0 | 0 | 0 | 0 |
| TBAES2009387 | 0 | 0 | 7.521 | 0 | 0 | 0 | 0.349 |
| TCERX2000171 | 0 | 0 | 0 | 100 | 0 | 0 | 0 |
| TCERX2000431 | 0 | 0 | 0 | 44.331 | 0 | 0 | 0 |
| TCERX2000613 | 0 | 0 | 0 | 85.503 | 0 | 0 | 0 |
| TCERX2000998 | 0 | 0 | 0 | 100 | 0 | 0 | 0 |
| TCERX2002644 | 0 | 0 | 24.136 | 72.407 | 0 | 0 | 0 |
| TCOLN1000037 | 0 | 0 | 0 | 0 | 100 | 0 | 0 |
| TCOLN1000180 | 0 | 0 | 0 | 0 | 51.906 | 0 | 0 |
| TCOLN2000139 | 0 | 0 | 0 | 0 | 100 | 0 | 0 |
| TCOLN2000236 | 0 | 0 | 0 | 0 | 100 | 0 | 0 |
| TCOLN2000641 | 0 | 0 | 0 | 0 | 100 | 0 | 0 |
| TCOLN2001268 | 0 | 0 | 0 | 0 | 100 | 0 | 0 |
| TCOLN2002047 | 0 | 0 | 0 | 0 | 100 | 0 | 0 |
| TCOLN2002278 | 0 | 0 | 0 | 0 | 100 | 0 | 0 |
| TESOP1000035 | 0 | 0 | 0 | 0 | 0 | 30.261 | 0 |
| TESOP1000127 | 0 | 0 | 0 | 0 | 0 | 65.101 | 0 |
| TESOP1000160 | 0 | 0 | 0 | 0 | 0 | 100 | 0 |
| TESOP2000083 | 0 | 0 | 0 | 0 | 0 | 100 | 0 |
| TESOP2000090 | 0 | 0 | 0 | 0 | 0 | 100 | 0 |
| TESOP2000205 | 0 | 0 | 0 | 0 | 0 | 100 | 0 |
| TESOP2000312 | 0 | 0 | 0 | 0 | 0 | 66.097 | 0 |
| TESOP2000390 | 0 | 0 | 0 | 0 | 0 | 100 | 0 |
| TESOP2000400 | 0 | 0 | 0 | 0 | 0 | 100 | 0 |
| TESOP2000510 | 0 | 0 | 0 | 0 | 0 | 100 | 0 |
| TESOP2000527 | 0 | 0 | 0 | 0 | 0 | 70.604 | 0 |
| TESOP2000569 | 0 | 0 | 0 | 0 | 0 | 100 | 0 |
| TESOP2000739 | 0 | 0 | 0 | 0 | 0 | 100 | 0 |
| TESOP2000801 | 0 | 0 | 0 | 0 | 0 | 100 | 0 |
| TESOP2001122 | 0 | 0 | 0 | 0 | 0 | 100 | 0 |
| TESOP2001166 | 0 | 0 | 0 | 0 | 0 | 100 | 0 |
| TESOP2001345 | 0 | 0 | 0 | 0 | 0 | 100 | 0 |
| TESOP2001474 | 0 | 0 | 0 | 0 | 0 | 100 | 0 |
| TESOP2001796 | 0 | 0 | 0 | 0 | 0 | 100 | 0 |
| TESOP2001818 | 0 | 0 | 0 | 0 | 0 | 100 | 0 |
| TESOP2001849 | 0 | 0 | 0 | 0 | 0 | 100 | 0 |
| TESOP2001865 | 0 | 0 | 0 | 0 | 0 | 100 | 0 |
| TESOP2001901 | 0 | 0 | 0 | 0 | 0 | 100 | 0 |
| TESOP2001953 | 0 | 0 | 0 | 0 | 0 | 89.137 | 0 |
| TESOP2002005 | 0 | 0 | 0 | 0 | 0 | 100 | 0 |
| TESOP2002110 | 0 | 0 | 0 | 0 | 0 | 100 | 0 |
| TESOP2002150 | 0 | 0 | 0 | 0 | 0 | 100 | 0 |
| TESOP2002273 | 0 | 0 | 0 | 0 | 0 | 27.770 | 2.653 |
| TESOP2002451 | 0 | 0 | 0 | 0 | 0 | 16.251 | 0 |
| TESOP2002489 | 0 | 0 | 0 | 0 | 0 | 100 | 0 |
| TESOP2002539 | 0 | 0 | 0 | 0 | 0 | 100 | 0 |
| TESOP2002545 | 0 | 0 | 0 | 0 | 0 | 100 | 0 |
| TESOP2002804 | 0 | 0 | 0 | 0 | 0 | 100 | 0 |
| TESOP2002950 | 0 | 0 | 0 | 0 | 0 | 90.601 | 0 |
| TESOP2003273 | 0 | 0 | 0 | 0 | 0 | 100 | 0 |
| TESOP2003308 | 0 | 0 | 0 | 0 | 0 | 100 | 0 |
| TESOP2003553 | 0 | 0 | 0 | 0 | 0 | 100 | 0 |
| TESOP2003753 | 0 | 0 | 0 | 0 | 0 | 100 | 0 |
| TESOP2004110 | 0 | 0 | 0 | 0 | 0 | 100 | 0 |
| TESOP2004114 | 0 | 0 | 0 | 0 | 0 | 100 | 0 |
| TESOP2004173 | 0 | 0 | 0 | 0 | 0 | 100 | 0 |
| TESOP2004214 | 0 | 0 | 0 | 0 | 0 | 100 | 0 |
| TESOP2005199 | 0 | 0 | 0 | 0 | 0 | 100 | 0 |
| TESOP2005285 | 0 | 0 | 0 | 0 | 0 | 100 | 0 |
| TESOP2005485 | 0 | 0 | 0 | 0 | 0 | 100 | 0 |
| TESOP2005498 | 0 | 0 | 0 | 0 | 0 | 100 | 0 |
| TESOP2005579 | 0 | 0 | 0 | 0 | 0 | 100 | 0 |
| TESOP2006041 | 0 | 0 | 0 | 0 | 0 | 100 | 0 |
| TESOP2006053 | 0 | 0 | 0 | 0 | 0 | 50.390 | 0 |
| TESOP2006060 | 0 | 0 | 0 | 0 | 0 | 100 | 0 |
| TESOP2006068 | 0 | 0 | 0 | 0 | 0 | 100 | 0 |
| TESOP2006110 | 0 | 0 | 0 | 0 | 0 | 100 | 0 |
| TESOP2006398 | 0 | 0 | 0 | 0 | 0 | 100 | 0 |
| TESOP2006670 | 0 | 0 | 0 | 0 | 0 | 79.728 | 0 |
| TESOP2006704 | 0 | 0 | 0 | 0 | 0 | 100 | 0 |
| TESOP2006746 | 0 | 0 | 0 | 0 | 0 | 100 | 0 |
| TESOP2006865 | 0 | 0 | 0 | 0 | 0 | 100 | 0 |
| TESOP2006893 | 0 | 0 | 0 | 0 | 0 | 58.778 | 0 |
| TESOP2007041 | 0 | 0 | 0 | 0 | 0 | 100 | 0 |
| TESOP2007052 | 0 | 0 | 0 | 0 | 0 | 100 | 0 |
| TESOP2007262 | 0 | 0 | 0 | 0 | 0 | 100 | 0 |
| TESOP2007384 | 0 | 0 | 0 | 0 | 0 | 13.734 | 0 |
| TESOP2007636 | 0 | 0 | 0 | 0 | 0 | 100 | 0 |
| TESOP2007674 | 0 | 0 | 0 | 0 | 0 | 100 | 0 |
| TESOP2007688 | 0 | 0 | 0 | 0 | 0 | 100 | 0 |
| TESOP2007725 | 0 | 0 | 0 | 0 | 0 | 100 | 0 |
| TESOP2007912 | 0 | 0 | 0 | 0 | 0 | 78.739 | 0 |
| TESOP2007978 | 0 | 0 | 0 | 0 | 0 | 100 | 0 |
| TESOP2008127 | 0 | 0 | 0 | 0 | 0 | 100 | 0 |
| TESOP2008552 | 0 | 0 | 0 | 0 | 0 | 100 | 0 |
| TESOP2008556 | 0 | 0 | 0 | 0 | 0 | 100 | 0 |
| TESOP2008563 | 0 | 0 | 0 | 0 | 0 | 100 | 0 |
| TESOP2008820 | 0 | 0 | 0 | 0 | 0 | 65.779 | 0 |
| TESOP2009121 | 0 | 0 | 0 | 0 | 0 | 100 | 0 |
| TESOP2009201 | 0 | 0 | 0 | 0 | 0 | 100 | 0 |
| TESOP2009247 | 0 | 0 | 0 | 0 | 0 | 100 | 0 |
| TESOP2009555 | 0 | 0 | 0 | 0 | 0 | 100 | 0 |
| TESTI1000018 | 0 | 0 | 0 | 0 | 0 | 0 | 100 |
| TESTI1000019 | 0 | 0 | 0 | 0 | 0 | 0 | 100 |
| TESTI1000023 | 1.809 | 0 | 0 | 0 | 0 | 0 | 1.645 |
| TESTI1000025 | 0 | 0 | 0 | 0 | 0 | 0 | 100 |
| TESTI1000029 | 0 | 0 | 0 | 0 | 0 | 0 | 100 |
| TESTI1000030 | 0 | 0 | 0 | 0 | 0 | 0 | 100 |
| TESTI1000042 | 0 | 0 | 0 | 0 | 0 | 0 | 100 |
| TESTI1000045 | 0 | 0 | 0 | 0 | 0 | 0 | 100 |
| TESTI1000051 | 0 | 0 | 0 | 0 | 0 | 0 | 40.852 |
| TESTI1000055 | 0 | 0 | 0 | 0 | 0 | 0 | 100 |
| TESTI1000064 | 0 | 0 | 0 | 0 | 0 | 0 | 82.736 |
| TESTI1000070 | 0 | 0 | 0 | 0 | 0 | 49.407 | 30.683 |
| TESTI1000085 | 0 | 0 | 0 | 0 | 0 | 0 | 100 |
| TESTI1000088 | 0 | 0 | 0 | 0 | 0 | 0 | 100 |
| TESTI1000093 | 0 | 0 | 0 | 0 | 0 | 0 | 100 |
| TESTI1000094 | 0 | 0 | 0 | 0 | 0 | 0 | 77.371 |
| TESTI1000096 | 0 | 0 | 0 | 0 | 0 | 0 | 11.435 |
| TESTI1000101 | 0 | 0 | 30.131 | 0 | 0 | 0 | 13.999 |
| TESTI1000109 | 0 | 0 | 0 | 0 | 0 | 0 | 100 |
| TESTI1000125 | 0 | 0 | 0 | 0 | 0 | 0 | 100 |
| TESTI1000127 | 0 | 0 | 0 | 0 | 0 | 0 | 100 |
| TESTI1000128 | 0 | 0 | 0 | 0 | 0 | 0 | 100 |
| TESTI1000131 | 0 | 0 | 0 | 0 | 0 | 0 | 100 |
| TESTI1000138 | 0 | 0 | 0 | 0 | 0 | 0 | 19.512 |
| TESTI1000142 | 0 | 0 | 0 | 0 | 0 | 0 | 30.873 |
| TESTI1000157 | 0 | 0 | 0 | 0 | 0 | 0 | 100 |
| TESTI1000163 | 0 | 0 | 0 | 0 | 0 | 0 | 2.693 |
| TESTI1000168 | 0 | 0 | 0 | 0 | 0 | 0 | 51.212 |
| TESTI1000169 | 0 | 0 | 0 | 0 | 0 | 0 | 100 |
| TESTI1000179 | 0 | 0 | 0 | 0 | 0 | 0 | 100 |
| TESTI1000183 | 0 | 0 | 0 | 0 | 0 | 0 | 100 |
| TESTI1000191 | 0 | 0 | 0 | 0 | 0 | 0 | 100 |
| TESTI1000257 | 0 | 0 | 0 | 0 | 0 | 0 | 100 |
| TESTI1000266 | 0 | 0 | 0 | 0 | 0 | 0 | 34.478 |
| TESTI1000319 | 0 | 0 | 0 | 0 | 0 | 0 | 100 |
| TESTI1000330 | 0 | 0 | 0 | 0 | 0 | 0 | 47.943 |
| TESTI1000348 | 0 | 0 | 0 | 0 | 0 | 0 | 100 |
| TESTI1000390 | 0 | 0 | 0 | 0 | 0 | 0 | 100 |
| TESTI1000391 | 0 | 0 | 0 | 0 | 0 | 0 | 100 |
| TESTI1000459 | 0 | 0 | 0 | 0 | 0 | 0 | 100 |
| TESTI1000491 | 0 | 0 | 0 | 0 | 0 | 0 | 100 |
| TESTI1000545 | 0 | 0 | 0 | 0 | 0 | 0 | 100 |
| TESTI2000006 | 0 | 0 | 0 | 0 | 0 | 0 | 6.464 |
| TESTI2000018 | 0 | 0 | 0 | 0 | 0 | 0 | 100 |
| TESTI2000044 | 0 | 0 | 0 | 0 | 0 | 0 | 100 |
| TESTI2000100 | 0 | 0 | 0 | 0 | 0 | 0 | 12.664 |
| TESTI2000117 | 0 | 0 | 0 | 0 | 0 | 0 | 100 |
| TESTI2000120 | 0 | 0 | 0 | 0 | 0 | 0 | 100 |
| TESTI2000147 | 0 | 0 | 0 | 0 | 0 | 0 | 100 |
| TESTI2000154 | 0 | 0 | 0 | 0 | 0 | 0 | 100 |
| TESTI2000172 | 0 | 0 | 0 | 0 | 0 | 0 | 5.592 |
| TESTI2000177 | 0 | 0 | 0 | 0 | 0 | 0 | 47.814 |
| TESTI2000179 | 0 | 0 | 0 | 0 | 0 | 0 | 23.946 |
| TESTI2000184 | 2.438 | 0 | 0 | 0 | 0 | 7.734 | 7.389 |
| TESTI2000207 | 0 | 0 | 0 | 0 | 0 | 0 | 100 |
| TESTI2000208 | 0 | 0 | 0 | 0 | 0 | 0 | 100 |
| TESTI2000221 | 0 | 0 | 0 | 0 | 0 | 0 | 100 |
| TESTI2000238 | 0 | 0 | 0 | 0 | 0 | 0 | 41.440 |
| TESTI2000253 | 0 | 0 | 0 | 0 | 0 | 0 | 61.294 |
| TESTI2000272 | 0 | 0 | 0 | 0 | 0 | 0 | 82.282 |
| TESTI2000278 | 0 | 0 | 0 | 0 | 0 | 0 | 100 |
| TESTI2000349 | 0 | 0 | 0 | 0 | 0 | 0 | 15.564 |
| TESTI2000356 | 0 | 0 | 0 | 0 | 0 | 0 | 22.925 |
| TESTI2000358 | 0 | 0 | 0 | 0 | 0 | 0 | 100 |
| TESTI2000372 | 0 | 0 | 0 | 0 | 0 | 0 | 100 |
| TESTI2000427 | 0 | 0 | 0 | 0 | 0 | 0 | 100 |
| TESTI2000431 | 0 | 0 | 0 | 0 | 0 | 0 | 63.646 |
| TESTI2000435 | 0 | 0 | 0 | 0 | 0 | 0 | 12.571 |
| TESTI2000443 | 0 | 0 | 0 | 0 | 0 | 0 | 100 |
| TESTI2000452 | 0 | 0 | 0 | 0 | 0 | 0 | 100 |
| TESTI2000462 | 0 | 0 | 0 | 0 | 0 | 0 | 100 |
| TESTI2000489 | 0 | 0 | 0 | 0 | 0 | 0 | 42.034 |
| TESTI2000520 | 0 | 0 | 0 | 0 | 79.762 | 0 | 2.459 |
| TESTI2000571 | 0 | 0 | 0 | 0 | 0 | 0 | 100 |
| TESTI2000591 | 0 | 0 | 12.846 | 0 | 0 | 0 | 4.775 |
| TESTI2000598 | 0 | 0 | 0 | 0 | 0 | 0 | 16.418 |
| TESTI2000600 | 0 | 0 | 0 | 0 | 0 | 0 | 24.263 |
| TESTI2000616 | 0 | 0 | 0 | 0 | 0 | 0 | 100 |
| TESTI2000627 | 0 | 0 | 0 | 0 | 0 | 0 | 13.773 |
| TESTI2000644 | 0 | 0 | 0 | 0 | 0 | 0 | 100 |
| TESTI2000671 | 0 | 0 | 0 | 0 | 0 | 0 | 100 |
| TESTI2000683 | 0 | 0 | 0 | 0 | 0 | 0 | 100 |
| TESTI2000689 | 0 | 0 | 0 | 0 | 0 | 0 | 100 |
| TESTI2000695 | 0 | 0 | 0 | 0 | 0 | 0 | 100 |
| TESTI2000699 | 0 | 0 | 0 | 0 | 0 | 0 | 100 |
| TESTI2000707 | 0 | 0 | 0 | 0 | 0 | 0 | 100 |
| TESTI2000745 | 0 | 0 | 0 | 0 | 0 | 0 | 100 |
| TESTI2000756 | 0 | 0 | 0 | 0 | 0 | 0 | 100 |
| TESTI2000762 | 0 | 0 | 0 | 0 | 0 | 0 | 100 |
| TESTI2000784 | 0 | 0 | 15.987 | 0 | 0 | 0 | 26.740 |
| TESTI2000808 | 0 | 0 | 0 | 0 | 0 | 0 | 100 |
| TESTI2000819 | 0 | 0 | 0 | 0 | 0 | 0 | 100 |
| TESTI2000830 | 0 | 0 | 0 | 0 | 0 | 0 | 100 |
| TESTI2000840 | 0 | 0 | 0 | 0 | 0 | 0 | 100 |
| TESTI2000849 | 0 | 0 | 0 | 0 | 0 | 0 | 70.219 |
| TESTI2000850 | 0 | 0 | 0 | 0 | 0 | 0 | 100 |
| TESTI2000874 | 0 | 0 | 0 | 0 | 0 | 0 | 8.171 |
| TESTI2000883 | 0 | 0 | 0 | 0 | 0 | 0 | 14.298 |
| TESTI2000909 | 0 | 0 | 0 | 0 | 0 | 0 | 8.520 |
| TESTI2000951 | 0 | 0 | 0 | 0 | 0 | 0 | 100 |
| TESTI2000970 | 0 | 0 | 0 | 0 | 0 | 0 | 6.355 |
| TESTI2000981 | 0 | 0 | 0 | 0 | 0 | 0 | 100 |
| TESTI2001008 | 0 | 0 | 0 | 0 | 0 | 0 | 100 |
| TESTI2001049 | 0 | 0 | 0 | 0 | 0 | 0 | 100 |
| TESTI2001082 | 0 | 0 | 0 | 0 | 0 | 0 | 100 |
| TESTI2001099 | 1.057 | 0 | 0 | 0 | 0 | 0 | 9.930 |
| TESTI2001120 | 0 | 0 | 0 | 0 | 0 | 0 | 0.632 |
| TESTI2001134 | 0 | 0 | 0 | 0 | 0 | 0 | 100 |
| TESTI2001141 | 0 | 0 | 0 | 0 | 0 | 0 | 49.381 |
| TESTI2001153 | 0 | 0 | 0 | 0 | 0 | 0 | 2.603 |
| TESTI2001180 | 0 | 0 | 0 | 0 | 0 | 0 | 100 |
| TESTI2001208 | 0 | 0 | 0 | 0 | 0 | 0 | 100 |
| TESTI2001229 | 0 | 0 | 0 | 0 | 0 | 0 | 100 |
| TESTI2001236 | 0 | 0 | 0 | 0 | 0 | 0 | 100 |
| TESTI2001237 | 0 | 0 | 0 | 0 | 0 | 0 | 100 |
| TESTI2001269 | 1.177 | 0 | 0 | 0 | 0 | 0 | 1.070 |
| TESTI2001306 | 0 | 0 | 0 | 0 | 0 | 0 | 100 |
| TESTI2001313 | 0 | 0 | 0 | 0 | 0 | 0 | 100 |
| TESTI2001330 | 0 | 0 | 0 | 0 | 0 | 0 | 100 |
| TESTI2001339 | 0 | 0 | 0 | 0 | 0 | 0 | 27.313 |
| TESTI2001345 | 0 | 0 | 0 | 0 | 0 | 0 | 100 |
| TESTI2001352 | 0 | 0 | 0 | 0 | 0 | 0 | 100 |
| TESTI2001364 | 0 | 0 | 0 | 0 | 0 | 0 | 100 |
| TESTI2001412 | 0 | 0 | 0 | 0 | 0 | 0 | 100 |
| TESTI2001420 | 0 | 0 | 0 | 0 | 0 | 0 | 35.404 |
| TESTI2001498 | 0 | 0 | 0 | 0 | 0 | 0 | 100 |
| TESTI2001511 | 0 | 0 | 0 | 0 | 0 | 0 | 1.144 |
| TESTI2001512 | 0 | 0 | 0 | 0 | 0 | 0 | 100 |
| TESTI2001518 | 0 | 0 | 0 | 0 | 0 | 0 | 100 |
| TESTI2001556 | 0 | 0 | 0 | 0 | 0 | 0 | 100 |
| TESTI2001593 | 0 | 0 | 0 | 0 | 0 | 0 | 100 |
| TESTI2001613 | 0 | 0 | 0 | 0 | 0 | 0 | 17.860 |
| TESTI2001621 | 0 | 0 | 0 | 0 | 0 | 0 | 100 |
| TESTI2001661 | 0 | 0 | 0 | 0 | 0 | 0 | 54.042 |
| TESTI2001665 | 0 | 0 | 0 | 0 | 0 | 0 | 100 |
| TESTI2001671 | 0 | 0 | 0 | 0 | 0 | 0 | 100 |
| TESTI2001697 | 0 | 0 | 0 | 0 | 0 | 0 | 100 |
| TESTI2001725 | 0 | 0 | 0 | 0 | 0 | 0 | 100 |
| TESTI2001758 | 0 | 0 | 0 | 0 | 0 | 0 | 100 |
| TESTI2001766 | 0 | 0 | 0 | 0 | 0 | 0 | 100 |
| TESTI2001792 | 0 | 0 | 0 | 0 | 0 | 0 | 100 |
| TESTI2001793 | 0 | 0 | 0 | 0 | 0 | 0 | 100 |
| TESTI2001795 | 0 | 0 | 0 | 0 | 0 | 0 | 100 |
| TESTI2001812 | 0 | 0 | 0 | 0 | 0 | 0 | 100 |
| TESTI2001815 | 76.742 | 0 | 0 | 0 | 0 | 0 | 23.258 |
| TESTI2001823 | 0 | 0 | 0 | 0 | 0 | 0 | 100 |
| TESTI2001826 | 0 | 0 | 0 | 0 | 0 | 0 | 58.511 |
| TESTI2001827 | 0 | 0 | 0 | 0 | 0 | 0 | 100 |
| TESTI2001829 | 0 | 0 | 0 | 0 | 0 | 0 | 1.665 |
| TESTI2001852 | 0 | 0 | 0 | 0 | 0 | 0 | 100 |
| TESTI2001862 | 0 | 0 | 0 | 0 | 0 | 0 | 1.728 |
| TESTI2001869 | 0 | 0 | 0 | 0 | 0 | 0 | 15.600 |
| TESTI2001879 | 0 | 0 | 0 | 0 | 0 | 0 | 36.852 |
| TESTI2001894 | 0 | 0 | 0 | 0 | 0 | 0 | 100 |
| TESTI2001898 | 0 | 0 | 0 | 0 | 0 | 0 | 39.051 |
| TESTI2001915 | 0 | 0 | 0 | 0 | 0 | 0 | 100 |
| TESTI2001950 | 0 | 0 | 0 | 0 | 0 | 0 | 100 |
| TESTI2001959 | 0 | 0 | 0 | 0 | 0 | 0 | 100 |
| TESTI2001968 | 0 | 0 | 0 | 0 | 0 | 0 | 100 |
| TESTI2001985 | 0 | 0 | 0 | 0 | 0 | 0 | 61.396 |
| TESTI2001991 | 0 | 0 | 0 | 0 | 0 | 0 | 100 |
| TESTI2002002 | 0 | 0 | 0 | 0 | 0 | 0 | 100 |
| TESTI2002012 | 0 | 0 | 0 | 0 | 0 | 0 | 100 |
| TESTI2002036 | 0 | 0 | 0 | 0 | 0 | 0 | 100 |
| TESTI2002048 | 0 | 0 | 0 | 0 | 0 | 0 | 100 |
| TESTI2002057 | 0 | 0 | 0 | 0 | 0 | 0 | 100 |
| TESTI2002081 | 0 | 0 | 0 | 0 | 0 | 0 | 100 |
| TESTI2002090 | 0 | 0 | 0 | 0 | 0 | 0 | 100 |
| TESTI2002105 | 0 | 0 | 0 | 0 | 0 | 0 | 85.371 |
| TESTI2002149 | 0 | 0 | 0 | 0 | 0 | 0 | 2.187 |
| TESTI2002216 | 0 | 0 | 0 | 0 | 0 | 0 | 100 |
| TESTI2002223 | 0 | 0 | 0 | 0 | 0 | 0 | 17.976 |
| TESTI2002245 | 0 | 0 | 0 | 0 | 0 | 0 | 100 |
| TESTI2002251 | 0 | 0 | 0 | 0 | 0 | 0 | 100 |
| TESTI2002256 | 0 | 0 | 0 | 0 | 0 | 0 | 22.675 |
| TESTI2002264 | 0 | 0 | 0 | 0 | 0 | 0 | 100 |
| TESTI2002282 | 0 | 0 | 0 | 0 | 0 | 0 | 100 |
| TESTI2002293 | 0 | 0 | 0 | 0 | 0 | 0 | 100 |
| TESTI2002294 | 0 | 0 | 0 | 0 | 0 | 0 | 100 |
| TESTI2002326 | 0 | 0 | 0 | 0 | 0 | 0 | 100 |
| TESTI2002351 | 0 | 0 | 0 | 0 | 0 | 0 | 100 |
| TESTI2002361 | 0 | 0 | 0 | 0 | 0 | 0 | 100 |
| TESTI2002365 | 0 | 0 | 7.682 | 0 | 0 | 0 | 7.138 |
| TESTI2002369 | 0 | 0 | 0 | 0 | 0 | 0 | 19.645 |
| TESTI2002406 | 0 | 0 | 0 | 0 | 0 | 0 | 100 |
| TESTI2002415 | 0 | 0 | 0 | 0 | 0 | 0 | 100 |
| TESTI2002423 | 0 | 0 | 0 | 0 | 0 | 0 | 100 |
| TESTI2002453 | 0 | 0 | 0 | 0 | 0 | 0 | 100 |
| TESTI2002461 | 0 | 0 | 0 | 0 | 0 | 0 | 100 |
| TESTI2002465 | 0 | 0 | 0 | 0 | 0 | 0 | 100 |
| TESTI2002467 | 0 | 0 | 0 | 0 | 0 | 0 | 15.347 |
| TESTI2002498 | 0 | 0 | 0 | 0 | 0 | 0 | 100 |
| TESTI2002516 | 0 | 0 | 0 | 0 | 0 | 0 | 100 |
| TESTI2002544 | 0 | 0 | 0 | 0 | 0 | 0 | 100 |
| TESTI2002578 | 0 | 0 | 0 | 0 | 0 | 0 | 100 |
| TESTI2002580 | 0 | 0 | 0 | 0 | 0 | 0 | 100 |
| TESTI2002618 | 0 | 0 | 0 | 0 | 0 | 0 | 100 |
| TESTI2002632 | 0 | 0 | 0 | 0 | 0 | 0 | 7.077 |
| TESTI2002676 | 0 | 0 | 0 | 0 | 0 | 0 | 100 |
| TESTI2002690 | 0 | 0 | 0 | 0 | 0 | 0 | 100 |
| TESTI2002696 | 0 | 0 | 0 | 0 | 0 | 0 | 8.489 |
| TESTI2002698 | 0 | 0 | 0 | 0 | 0 | 0 | 100 |
| TESTI2002700 | 0 | 0 | 0 | 0 | 0 | 0 | 100 |
| TESTI2002707 | 0 | 0 | 0 | 0 | 0 | 0 | 5.415 |
| TESTI2002724 | 0 | 0 | 0 | 0 | 0 | 0 | 100 |
| TESTI2002729 | 0 | 0 | 0 | 0 | 0 | 0 | 100 |
| TESTI2002752 | 0 | 0 | 0 | 0 | 0 | 0 | 100 |
| TESTI2002753 | 0 | 0 | 0 | 0 | 0 | 0 | 100 |
| TESTI2002789 | 0 | 0 | 0 | 0 | 0 | 0 | 100 |
| TESTI2002802 | 0 | 0 | 0 | 0 | 0 | 0 | 100 |
| TESTI2002806 | 0 | 0 | 0 | 0 | 0 | 0 | 100 |
| TESTI2002840 | 0 | 0 | 0 | 0 | 0 | 0 | 26.135 |
| TESTI2002866 | 0 | 0 | 0 | 0 | 0 | 0 | 13.910 |
| TESTI2002877 | 0 | 0 | 0 | 0 | 0 | 0 | 24.389 |
| TESTI2002910 | 0 | 0 | 0 | 0 | 0 | 0 | 100 |
| TESTI2002912 | 0 | 0 | 0 | 0 | 0 | 0 | 100 |
| TESTI2002928 | 0 | 0 | 0 | 0 | 0 | 0 | 100 |
| TESTI2002937 | 0 | 0 | 0 | 0 | 0 | 0 | 100 |
| TESTI2002965 | 0 | 0 | 0 | 0 | 0 | 0 | 31.989 |
| TESTI2002993 | 0 | 0 | 0 | 0 | 0 | 0 | 100 |
| TESTI2003005 | 0 | 0 | 0 | 0 | 0 | 0 | 1.986 |
| TESTI2003020 | 0 | 0 | 0 | 0 | 0 | 0 | 100 |
| TESTI2003031 | 0 | 0 | 0 | 0 | 0 | 0 | 100 |
| TESTI2003037 | 0 | 0 | 0 | 0 | 0 | 0 | 100 |
| TESTI2003044 | 0 | 0 | 64.204 | 0 | 0 | 0 | 35.796 |
| TESTI2003059 | 0 | 0 | 0 | 0 | 0 | 0 | 100 |
| TESTI2003061 | 0 | 0 | 0 | 0 | 0 | 0 | 67.719 |
| TESTI2003071 | 0 | 0 | 0 | 0 | 0 | 0 | 2.281 |
| TESTI2003074 | 0 | 0 | 0 | 0 | 0 | 0 | 100 |
| TESTI2003089 | 0 | 0 | 0 | 0 | 0 | 0 | 100 |
| TESTI2003104 | 0 | 0 | 0 | 0 | 0 | 0 | 100 |
| TESTI2003109 | 0 | 0 | 0 | 0 | 0 | 0 | 74.476 |
| TESTI2003117 | 0 | 0 | 0 | 0 | 0 | 0 | 100 |
| TESTI2003127 | 0 | 0 | 0 | 0 | 0 | 0 | 6.980 |
| TESTI2003130 | 0 | 0 | 0 | 0 | 0 | 0 | 100 |
| TESTI2003131 | 0 | 0 | 0 | 0 | 0 | 0 | 100 |
| TESTI2003139 | 0 | 0 | 0 | 0 | 0 | 0 | 100 |
| TESTI2003141 | 0 | 0 | 0 | 0 | 0 | 0 | 47.944 |
| TESTI2003152 | 0 | 0 | 0 | 0 | 0 | 0 | 100 |
| TESTI2003181 | 0 | 0 | 0 | 0 | 0 | 0 | 100 |
| TESTI2003193 | 0 | 0 | 0 | 0 | 0 | 0 | 100 |
| TESTI2003196 | 0 | 0 | 0 | 0 | 0 | 0 | 100 |
| TESTI2003228 | 0 | 0 | 0 | 0 | 0 | 0 | 100 |
| TESTI2003236 | 0 | 0 | 0 | 0 | 0 | 0 | 100 |
| TESTI2003255 | 0 | 0 | 0 | 0 | 0 | 0 | 100 |
| TESTI2003258 | 0 | 0 | 0 | 0 | 0 | 0 | 100 |
| TESTI2003277 | 0 | 0 | 0 | 0 | 0 | 0 | 100 |
| TESTI2003280 | 0 | 0 | 0 | 0 | 0 | 0 | 8.044 |
| TESTI2003299 | 0 | 0 | 0 | 0 | 0 | 0 | 100 |
| TESTI2003311 | 0 | 0 | 0 | 0 | 0 | 0 | 100 |
| TESTI2003321 | 0 | 0 | 0 | 0 | 0 | 0 | 100 |
| TESTI2003325 | 0 | 0 | 29.771 | 0 | 0 | 0 | 5.533 |
| TESTI2003327 | 0 | 0 | 0 | 0 | 0 | 0 | 100 |
| TESTI2003330 | 0 | 0 | 0 | 0 | 0 | 0 | 100 |
| TESTI2003347 | 0 | 0 | 0 | 0 | 0 | 0 | 100 |
| TESTI2003354 | 0 | 0 | 0 | 0 | 0 | 0 | 100 |
| TESTI2003356 | 0 | 0 | 0 | 0 | 0 | 0 | 100 |
| TESTI2003372 | 0 | 0 | 0 | 0 | 0 | 0 | 100 |
| TESTI2003376 | 0 | 0 | 0 | 0 | 0 | 0 | 100 |
| TESTI2003413 | 0 | 0 | 0 | 0 | 0 | 0 | 5.106 |
| TESTI2003418 | 0 | 0 | 0 | 0 | 0 | 0 | 100 |
| TESTI2003419 | 0 | 0 | 0 | 0 | 0 | 0 | 100 |
| TESTI2003432 | 0 | 0 | 0 | 0 | 0 | 0 | 100 |
| TESTI2003454 | 0 | 0 | 0 | 0 | 0 | 0 | 3.471 |
| TESTI2003475 | 0 | 0 | 0 | 0 | 0 | 0 | 100 |
| TESTI2003476 | 0 | 0 | 0 | 0 | 0 | 0 | 100 |
| TESTI2003498 | 0 | 0 | 0 | 0 | 0 | 0 | 100 |
| TESTI2003512 | 0 | 0 | 0 | 0 | 0 | 0 | 100 |
| TESTI2003533 | 0 | 0 | 12.198 | 0 | 0 | 0 | 1.133 |
| TESTI2003539 | 0 | 0 | 0 | 0 | 0 | 0 | 15.564 |
| TESTI2003541 | 0 | 0 | 0 | 0 | 0 | 0 | 38.183 |
| TESTI2003545 | 0 | 0 | 0 | 0 | 0 | 0 | 100 |
| TESTI2003551 | 0 | 0 | 0 | 0 | 0 | 0 | 100 |
| TESTI2003574 | 0 | 0 | 0 | 0 | 0 | 0 | 100 |
| TESTI2003579 | 0 | 0 | 0 | 0 | 0 | 0 | 22.717 |
| TESTI2003580 | 0 | 0 | 0 | 0 | 0 | 0 | 100 |
| TESTI2003589 | 0 | 0 | 0 | 0 | 0 | 0 | 100 |
| TESTI2003596 | 8.986 | 0 | 0 | 0 | 0 | 0 | 76.255 |
| TESTI2003598 | 0 | 0 | 0 | 0 | 0 | 0 | 100 |
| TESTI2003615 | 0 | 0 | 0 | 0 | 0 | 0 | 100 |
| TESTI2003625 | 0 | 0 | 0 | 0 | 0 | 0 | 10.007 |
| TESTI2003638 | 0 | 0 | 0 | 0 | 0 | 0 | 100 |
| TESTI2003649 | 0 | 0 | 0 | 0 | 0 | 0 | 100 |
| TESTI2003692 | 0 | 0 | 0 | 0 | 0 | 0 | 39.051 |
| TESTI2003718 | 0 | 0 | 0 | 0 | 0 | 0 | 100 |
| TESTI2003727 | 0 | 0 | 0 | 0 | 0 | 0 | 100 |
| TESTI2003756 | 0 | 0 | 0 | 0 | 0 | 0 | 20.574 |
| TESTI2003768 | 0 | 0 | 0 | 0 | 0 | 0 | 100 |
| TESTI2003781 | 0 | 0 | 0 | 0 | 0 | 0 | 100 |
| TESTI2003787 | 0 | 0 | 0 | 0 | 0 | 0 | 100 |
| TESTI2003824 | 0 | 0 | 0 | 0 | 0 | 0 | 100 |
| TESTI2003827 | 0 | 0 | 0 | 0 | 0 | 0 | 100 |
| TESTI2003894 | 0 | 0 | 0 | 0 | 0 | 0 | 100 |
| TESTI2003911 | 0 | 0 | 0 | 0 | 0 | 0 | 100 |
| TESTI2003914 | 0 | 0 | 0 | 0 | 0 | 0 | 100 |
| TESTI2003940 | 0 | 0 | 0 | 0 | 0 | 0 | 100 |
| TESTI2003962 | 0 | 0 | 0 | 0 | 0 | 0 | 100 |
| TESTI2003998 | 0 | 0 | 0 | 0 | 0 | 0 | 100 |
| TESTI2004000 | 0 | 0 | 0 | 0 | 0 | 0 | 4.583 |
| TESTI2004031 | 0 | 0 | 5.640 | 0 | 0 | 0 | 0.262 |
| TESTI2004073 | 0 | 0 | 0 | 0 | 0 | 0 | 100 |
| TESTI2004080 | 0 | 0 | 0 | 0 | 0 | 0 | 3.190 |
| TESTI2004085 | 0 | 0 | 0 | 0 | 0 | 0 | 100 |
| TESTI2004089 | 0 | 0 | 0 | 0 | 0 | 0 | 100 |
| TESTI2004111 | 0 | 0 | 0 | 0 | 0 | 0 | 100 |
| TESTI2004119 | 0 | 0 | 0 | 0 | 0 | 0 | 100 |
| TESTI2004122 | 0 | 0 | 0 | 0 | 0 | 0 | 9.979 |
| TESTI2004153 | 0 | 0 | 0 | 0 | 0 | 0 | 5.672 |
| TESTI2004163 | 0 | 0 | 0 | 0 | 0 | 0 | 100 |
| TESTI2004169 | 0 | 0 | 0 | 0 | 0 | 0 | 100 |
| TESTI2004207 | 0 | 0 | 0 | 0 | 0 | 0 | 100 |
| TESTI2004215 | 0 | 0 | 0 | 0 | 0 | 0 | 100 |
| TESTI2004227 | 0 | 0 | 0 | 0 | 0 | 0 | 100 |
| TESTI2004229 | 0 | 0 | 0 | 0 | 0 | 0 | 100 |
| TESTI2004243 | 0 | 0 | 0 | 0 | 0 | 0 | 100 |
| TESTI2004255 | 0 | 0 | 0 | 0 | 0 | 0 | 100 |
| TESTI2004287 | 0 | 0 | 0 | 0 | 0 | 0 | 100 |
| TESTI2004295 | 0 | 0 | 0 | 0 | 0 | 0 | 5.353 |
| TESTI2004313 | 0 | 0 | 0 | 0 | 0 | 0 | 100 |
| TESTI2004318 | 0 | 0 | 0 | 0 | 0 | 0 | 100 |
| TESTI2004322 | 0 | 0 | 0 | 0 | 0 | 0 | 100 |
| TESTI2004391 | 0 | 0 | 0 | 0 | 0 | 0 | 71.774 |
| TESTI2004399 | 0 | 0 | 0 | 0 | 0 | 0 | 100 |
| TESTI2004423 | 0 | 0 | 0 | 0 | 0 | 0 | 100 |
| TESTI2004431 | 0 | 0 | 0 | 0 | 0 | 0 | 32.808 |
| TESTI2004432 | 0 | 0 | 0 | 0 | 0 | 0 | 36.852 |
| TESTI2004452 | 0 | 0 | 0 | 0 | 0 | 0 | 100 |
| TESTI2004459 | 0 | 0 | 0 | 0 | 0 | 0 | 100 |
| TESTI2004490 | 0 | 0 | 0 | 0 | 0 | 0 | 100 |
| TESTI2004539 | 0 | 0 | 0 | 0 | 0 | 0 | 100 |
| TESTI2004552 | 0 | 0 | 0 | 0 | 0 | 0 | 100 |
| TESTI2004560 | 0 | 0 | 0 | 0 | 0 | 0 | 100 |
| TESTI2004574 | 0 | 0 | 0 | 0 | 0 | 0 | 100 |
| TESTI2004601 | 0 | 0 | 0 | 0 | 0 | 0 | 100 |
| TESTI2004611 | 0 | 0 | 0 | 0 | 0 | 0 | 100 |
| TESTI2004649 | 0 | 0 | 0 | 0 | 0 | 0 | 100 |
| TESTI2004654 | 0 | 0 | 0 | 0 | 0 | 0 | 100 |
| TESTI2004675 | 0 | 0 | 0 | 0 | 0 | 0 | 100 |
| TESTI2004687 | 0 | 0 | 0 | 0 | 0 | 0 | 100 |
| TESTI2004689 | 0 | 0 | 0 | 0 | 0 | 0 | 100 |
| TESTI2004700 | 0 | 0 | 0 | 0 | 0 | 0 | 100 |
| TESTI2004706 | 0 | 0 | 0 | 0 | 0 | 0 | 100 |
| TESTI2004712 | 0 | 0 | 0 | 0 | 0 | 0 | 0.739 |
| TESTI2004734 | 0 | 0 | 0 | 0 | 0 | 0 | 22.416 |
| TESTI2004737 | 0 | 0 | 24.921 | 0 | 0 | 0 | 5.789 |
| TESTI2004783 | 0 | 0 | 0 | 0 | 0 | 0 | 100 |
| TESTI2004791 | 0 | 0 | 0 | 0 | 0 | 0 | 100 |
| TESTI2004793 | 0 | 0 | 0 | 0 | 0 | 0 | 100 |
| TESTI2004860 | 0 | 0 | 0 | 0 | 0 | 0 | 100 |
| TESTI2004906 | 0 | 0 | 0 | 0 | 0 | 0 | 100 |
| TESTI2004920 | 0 | 0 | 0 | 0 | 0 | 0 | 100 |
| TESTI2004929 | 0 | 0 | 0 | 0 | 0 | 0 | 1.856 |
| TESTI2004936 | 0 | 0 | 0 | 0 | 0 | 0 | 100 |
| TESTI2004941 | 0 | 0 | 0 | 0 | 0 | 0 | 100 |
| TESTI2004971 | 0 | 0 | 0 | 0 | 0 | 0 | 100 |
| TESTI2004982 | 0 | 0 | 0 | 0 | 0 | 0 | 100 |
| TESTI2004993 | 0 | 0 | 0 | 0 | 0 | 0 | 100 |
| TESTI2004994 | 0 | 0 | 0 | 0 | 0 | 0 | 100 |
| TESTI2004999 | 0 | 0 | 0 | 0 | 0 | 0 | 100 |
| TESTI2005017 | 0 | 0 | 0 | 0 | 0 | 0 | 62.603 |
| TESTI2005025 | 0 | 0 | 0 | 0 | 0 | 0 | 100 |
| TESTI2005040 | 0 | 0 | 0 | 0 | 0 | 0 | 41.440 |
| TESTI2005060 | 0 | 0 | 0 | 0 | 0 | 0 | 100 |
| TESTI2005072 | 0 | 0 | 0 | 0 | 0 | 0 | 96.718 |
| TESTI2005085 | 0 | 0 | 0 | 0 | 0 | 0 | 100 |
| TESTI2005112 | 0 | 0 | 0 | 0 | 0 | 0 | 12.718 |
| TESTI2005120 | 0 | 0 | 0 | 0 | 0 | 0 | 100 |
| TESTI2005153 | 0 | 0 | 0 | 0 | 0 | 0 | 100 |
| TESTI2005155 | 0 | 0 | 0 | 0 | 0 | 0 | 100 |
| TESTI2005173 | 0 | 0 | 0 | 0 | 0 | 0 | 47.121 |
| TESTI2005267 | 0 | 0 | 0 | 0 | 0 | 0 | 100 |
| TESTI2005268 | 0 | 0 | 0 | 0 | 0 | 0 | 100 |
| TESTI2005280 | 0 | 0 | 0 | 0 | 0 | 0 | 100 |
| TESTI2005307 | 0 | 0 | 0 | 0 | 0 | 39.547 | 60.453 |
| TESTI2005326 | 0 | 0 | 0 | 0 | 0 | 0 | 14.987 |
| TESTI2005363 | 0 | 0 | 0 | 0 | 0 | 0 | 100 |
| TESTI2005376 | 0 | 0 | 0 | 0 | 0 | 0 | 100 |
| TESTI2005378 | 0 | 0 | 0 | 0 | 0 | 0 | 100 |
| TESTI2005380 | 0 | 0 | 0 | 0 | 0 | 0 | 100 |
| TESTI2005391 | 0 | 0 | 0 | 0 | 0 | 0 | 100 |
| TESTI2005395 | 0 | 0 | 0 | 0 | 0 | 0 | 51.276 |
| TESTI2005396 | 0 | 0 | 0 | 0 | 0 | 0 | 19.291 |
| TESTI2005408 | 3.821 | 0 | 0 | 0 | 0 | 0 | 2.316 |
| TESTI2005415 | 0 | 0 | 0 | 0 | 0 | 0 | 100 |
| TESTI2005470 | 0 | 0 | 0 | 0 | 0 | 0 | 100 |
| TESTI2005492 | 0 | 0 | 0 | 0 | 0 | 0 | 100 |
| TESTI2005564 | 0 | 0 | 25.762 | 0 | 0 | 0 | 11.970 |
| TESTI2005568 | 0 | 0 | 0 | 0 | 0 | 0 | 100 |
| TESTI2005584 | 0 | 0 | 0 | 0 | 0 | 0 | 100 |
| TESTI2005588 | 0 | 0 | 0 | 0 | 0 | 0 | 100 |
| TESTI2005590 | 0 | 0 | 0 | 0 | 0 | 0 | 100 |
| TESTI2005603 | 0 | 0 | 0 | 0 | 0 | 0 | 100 |
| TESTI2005609 | 0 | 0 | 0 | 0 | 0 | 0 | 100 |
| TESTI2005610 | 0 | 0 | 0 | 0 | 0 | 0 | 100 |
| TESTI2005630 | 0 | 0 | 0 | 0 | 0 | 0 | 100 |
| TESTI2005672 | 0 | 0 | 0 | 0 | 0 | 0 | 100 |
| TESTI2005683 | 0 | 0 | 0 | 0 | 0 | 0 | 100 |
| TESTI2005690 | 0 | 0 | 0 | 0 | 0 | 0 | 100 |
| TESTI2005692 | 0 | 0 | 0 | 0 | 0 | 0 | 36.852 |
| TESTI2005703 | 0 | 0 | 0 | 0 | 0 | 0 | 100 |
| TESTI2005720 | 0 | 0 | 0 | 0 | 0 | 0 | 100 |
| TESTI2005731 | 0 | 0 | 0 | 0 | 0 | 0 | 49.675 |
| TESTI2005739 | 0 | 0 | 0 | 0 | 0 | 0 | 100 |
| TESTI2005742 | 0 | 0 | 0 | 0 | 0 | 0 | 13.331 |
| TESTI2005743 | 0 | 0 | 0 | 0 | 0 | 0 | 1.436 |
| TESTI2005759 | 0 | 0 | 0 | 0 | 0 | 6.399 | 3.057 |
| TESTI2005767 | 0 | 0 | 0 | 0 | 0 | 0 | 92.965 |
| TESTI2005775 | 0 | 0 | 0 | 0 | 0 | 0 | 100 |
| TESTI2005784 | 0 | 0 | 0 | 0 | 0 | 0 | 1.944 |
| TESTI2005788 | 0 | 0 | 0 | 0 | 0 | 0 | 21.162 |
| TESTI2005827 | 0 | 0 | 0 | 0 | 0 | 0 | 100 |
| TESTI2005835 | 0 | 0 | 0 | 0 | 0 | 0 | 86.638 |
| TESTI2005860 | 0 | 0 | 0 | 0 | 0 | 0 | 100 |
| TESTI2005892 | 0 | 0 | 0 | 0 | 0 | 0 | 100 |
| TESTI2005908 | 6.792 | 0 | 0 | 0 | 0 | 0 | 1.029 |
| TESTI2005911 | 0 | 0 | 0 | 0 | 0 | 0 | 100 |
| TESTI2005933 | 0 | 0 | 0 | 0 | 0 | 0 | 100 |
| TESTI2005937 | 0 | 0 | 0 | 0 | 0 | 0 | 100 |
| TESTI2005948 | 0 | 0 | 0 | 0 | 0 | 0 | 100 |
| TESTI2005979 | 0 | 0 | 0 | 0 | 0 | 0 | 100 |
| TESTI2005981 | 0 | 0 | 0 | 0 | 0 | 0 | 20.358 |
| TESTI2005982 | 0 | 0 | 0 | 0 | 0 | 0 | 100 |
| TESTI2005984 | 0 | 0 | 0 | 0 | 0 | 0 | 100 |
| TESTI2005986 | 0 | 0 | 0 | 0 | 0 | 0 | 100 |
| TESTI2005987 | 0 | 0 | 0 | 0 | 0 | 0 | 100 |
| TESTI2006007 | 0 | 0 | 0 | 0 | 0 | 91.279 | 8.721 |
| TESTI2006008 | 0 | 0 | 0 | 0 | 0 | 0 | 100 |
| TESTI2006015 | 0 | 0 | 0 | 0 | 0 | 0 | 100 |
| TESTI2006016 | 0 | 0 | 0 | 0 | 0 | 0 | 100 |
| TESTI2006035 | 0 | 0 | 0 | 0 | 0 | 0 | 100 |
| TESTI2006040 | 0 | 0 | 0 | 0 | 0 | 0 | 100 |
| TESTI2006041 | 0 | 0 | 0 | 0 | 0 | 0 | 100 |
| TESTI2006045 | 0 | 0 | 0 | 0 | 0 | 0 | 100 |
| TESTI2006051 | 0 | 0 | 0 | 0 | 8.978 | 0 | 0.554 |
| TESTI2006067 | 0 | 0 | 0 | 0 | 0 | 0 | 100 |
| TESTI2006083 | 0 | 0 | 0 | 0 | 0 | 0 | 100 |
| TESTI2006097 | 0 | 0 | 0 | 0 | 0 | 0 | 13.774 |
| TESTI2006109 | 0 | 0 | 0 | 0 | 0 | 0 | 100 |
| TESTI2006111 | 0 | 0 | 0 | 0 | 0 | 0 | 13.477 |
| TESTI2006120 | 0 | 0 | 0 | 0 | 0 | 0 | 12.690 |
| TESTI2006125 | 0 | 0 | 0 | 0 | 0 | 0 | 100 |
| TESTI2006179 | 0 | 0 | 0 | 0 | 0 | 0 | 100 |
| TESTI2006209 | 0 | 0 | 0 | 0 | 0 | 0 | 100 |
| TESTI2006210 | 0 | 0 | 0 | 0 | 0 | 0 | 33.713 |
| TESTI2006212 | 0 | 0 | 0 | 0 | 0 | 0 | 100 |
| TESTI2006218 | 0 | 0 | 0 | 0 | 0 | 0 | 100 |
| TESTI2006238 | 0 | 0 | 0 | 0 | 0 | 0 | 100 |
| TESTI2006255 | 0 | 0 | 0 | 0 | 0 | 0 | 100 |
| TESTI2006258 | 0 | 0 | 0 | 0 | 0 | 0 | 6.703 |
| TESTI2006278 | 0 | 0 | 0 | 0 | 0 | 0 | 100 |
| TESTI2006333 | 0 | 0 | 0 | 0 | 0 | 0 | 100 |
| TESTI2006341 | 0 | 0 | 0 | 0 | 0 | 0 | 13.362 |
| TESTI2006360 | 0 | 0 | 0 | 0 | 0 | 0 | 100 |
| TESTI2006383 | 8.742 | 0 | 0 | 0 | 0 | 0 | 39.741 |
| TESTI2006409 | 0 | 0 | 0 | 0 | 0 | 0 | 39.051 |
| TESTI2006425 | 0 | 0 | 0 | 0 | 0 | 0 | 100 |
| TESTI2006437 | 0 | 0 | 0 | 0 | 0 | 0 | 100 |
| TESTI2006453 | 3.630 | 0 | 0 | 0 | 0 | 0 | 1.100 |
| TESTI2006457 | 0 | 0 | 0 | 0 | 0 | 0 | 100 |
| TESTI2006460 | 0 | 0 | 0 | 0 | 0 | 0 | 100 |
| TESTI2006465 | 0 | 0 | 0 | 0 | 0 | 0 | 100 |
| TESTI2006483 | 0 | 0 | 0 | 0 | 0 | 0 | 100 |
| TESTI2006499 | 0 | 0 | 0 | 0 | 0 | 0 | 100 |
| TESTI2006523 | 0 | 0 | 0 | 0 | 0 | 0 | 100 |
| TESTI2006543 | 0 | 0 | 0 | 0 | 0 | 0 | 100 |
| TESTI2006565 | 0 | 0 | 0 | 0 | 0 | 0 | 100 |
| TESTI2006572 | 0 | 0 | 0 | 0 | 0 | 0 | 100 |
| TESTI2006588 | 0 | 0 | 0 | 0 | 0 | 0 | 0.708 |
| TESTI2006615 | 0 | 0 | 0 | 0 | 0 | 0 | 72.403 |
| TESTI2006617 | 0 | 0 | 0 | 0 | 0 | 0 | 100 |
| TESTI2006624 | 0 | 0 | 0 | 0 | 0 | 0 | 100 |
| TESTI2006628 | 0 | 0 | 0 | 0 | 0 | 0 | 16.254 |
| TESTI2006633 | 0 | 0 | 0 | 0 | 0 | 0 | 52.424 |
| TESTI2006643 | 0 | 0 | 0 | 0 | 0 | 0 | 100 |
| TESTI2006648 | 0 | 0 | 0 | 0 | 0 | 0 | 100 |
| TESTI2006659 | 0 | 0 | 0 | 0 | 0 | 0 | 100 |
| TESTI2006660 | 0 | 0 | 0 | 0 | 0 | 0 | 100 |
| TESTI2006665 | 0 | 0 | 0 | 0 | 0 | 0 | 100 |
| TESTI2006667 | 0 | 0 | 0 | 0 | 0 | 0 | 100 |
| TESTI2006677 | 0 | 0 | 0 | 0 | 0 | 0 | 100 |
| TESTI2006720 | 0 | 0 | 0 | 0 | 0 | 0 | 43.944 |
| TESTI2006735 | 0 | 0 | 0 | 0 | 0 | 0 | 100 |
| TESTI2006744 | 0 | 0 | 0 | 0 | 0 | 0 | 100 |
| TESTI2006748 | 0 | 0 | 0 | 0 | 0 | 0 | 6.746 |
| TESTI2006774 | 0 | 0 | 0 | 0 | 0 | 0 | 47.943 |
| TESTI2006853 | 0 | 0 | 0 | 0 | 0 | 0 | 100 |
| TESTI2006866 | 0 | 0 | 0 | 0 | 0 | 0 | 100 |
| TESTI2006872 | 0 | 0 | 0 | 0 | 0 | 0 | 21.552 |
| TESTI2006877 | 0 | 0 | 0 | 0 | 0 | 0 | 52.051 |
| TESTI2006879 | 0 | 0 | 0 | 0 | 0 | 0 | 100 |
| TESTI2006894 | 0 | 0 | 0 | 0 | 0 | 0 | 100 |
| TESTI2006940 | 0 | 0 | 0 | 0 | 0 | 0 | 100 |
| TESTI2006955 | 0 | 0 | 0 | 0 | 0 | 0 | 100 |
| TESTI2006978 | 0 | 0 | 0 | 0 | 0 | 0 | 100 |
| TESTI2006979 | 0 | 0 | 0 | 0 | 0 | 0 | 100 |
| TESTI2006990 | 0 | 0 | 0 | 0 | 0 | 0 | 100 |
| TESTI2007040 | 0 | 0 | 0 | 0 | 0 | 0 | 100 |
| TESTI2007074 | 0 | 0 | 0 | 0 | 0 | 0 | 100 |
| TESTI2007078 | 0 | 0 | 0 | 0 | 0 | 0 | 100 |
| TESTI2007113 | 0 | 0 | 0 | 0 | 0 | 0 | 100 |
| TESTI2007152 | 0 | 0 | 0 | 0 | 0 | 0 | 100 |
| TESTI2007163 | 0 | 0 | 0 | 0 | 0 | 0 | 47.943 |
| TESTI2007183 | 0 | 0 | 0 | 0 | 0 | 0 | 100 |
| TESTI2007211 | 0 | 0 | 0 | 0 | 0 | 0 | 2.159 |
| TESTI2007220 | 0 | 0 | 0 | 0 | 0 | 0 | 100 |
| TESTI2007234 | 0 | 0 | 0 | 0 | 0 | 0 | 100 |
| TESTI2007311 | 0 | 0 | 0 | 0 | 0 | 0 | 2.515 |
| TESTI2007320 | 0 | 0 | 0 | 0 | 0 | 0 | 100 |
| TESTI2007346 | 0 | 0 | 0 | 0 | 0 | 0 | 100 |
| TESTI2007358 | 0 | 0 | 0 | 0 | 0 | 0 | 100 |
| TESTI2007402 | 0 | 0 | 0 | 0 | 0 | 0 | 100 |
| TESTI2007405 | 0 | 0 | 0 | 0 | 0 | 0 | 100 |
| TESTI2007407 | 0 | 0 | 0 | 0 | 0 | 0 | 2.006 |
| TESTI2007422 | 0 | 0 | 0 | 0 | 0 | 0 | 20.256 |
| TESTI2007452 | 0 | 0 | 0 | 0 | 0 | 0 | 100 |
| TESTI2007464 | 0 | 0 | 0 | 0 | 0 | 0 | 10.487 |
| TESTI2007466 | 0 | 0 | 0 | 0 | 0 | 0 | 100 |
| TESTI2007480 | 0 | 0 | 0 | 0 | 0 | 0 | 100 |
| TESTI2007490 | 0 | 0 | 0 | 0 | 0 | 0 | 100 |
| TESTI2007502 | 0 | 0 | 0 | 0 | 0 | 0 | 100 |
| TESTI2007524 | 0 | 0 | 0 | 0 | 0 | 0 | 100 |
| TESTI2007533 | 0 | 0 | 0 | 0 | 0 | 0 | 100 |
| TESTI2007576 | 0 | 0 | 0 | 0 | 0 | 0 | 100 |
| TESTI2007613 | 0 | 0 | 0 | 0 | 0 | 0 | 100 |
| TESTI2007657 | 0 | 0 | 0 | 0 | 0 | 0 | 100 |
| TESTI2007685 | 0 | 0 | 0 | 0 | 0 | 0 | 100 |
| TESTI2007692 | 0 | 0 | 0 | 0 | 0 | 0 | 100 |
| TESTI2007749 | 0 | 0 | 0 | 0 | 0 | 0 | 100 |
| TESTI2007750 | 0 | 0 | 0 | 0 | 0 | 0 | 100 |
| TESTI2007808 | 0 | 0 | 0 | 0 | 0 | 0 | 100 |
| TESTI2007814 | 0 | 0 | 0 | 0 | 0 | 0 | 100 |
| TESTI2007829 | 0 | 0 | 0 | 0 | 0 | 0 | 100 |
| TESTI2007864 | 0 | 0 | 0 | 0 | 0 | 0 | 43.944 |
| TESTI2007867 | 0 | 0 | 0 | 0 | 0 | 0 | 100 |
| TESTI2007872 | 0 | 0 | 0 | 0 | 0 | 0 | 100 |
| TESTI2007906 | 0 | 0 | 0 | 0 | 0 | 0 | 100 |
| TESTI2007922 | 0 | 0 | 0 | 0 | 0 | 0 | 10.332 |
| TESTI2007951 | 0 | 0 | 0 | 0 | 0 | 0 | 100 |
| TESTI2007972 | 0 | 0 | 0 | 0 | 0 | 0 | 100 |
| TESTI2007973 | 0 | 0 | 0 | 0 | 0 | 0 | 100 |
| TESTI2007998 | 0 | 0 | 0 | 0 | 0 | 0 | 4.134 |
| TESTI2008014 | 0 | 0 | 0 | 0 | 0 | 0 | 100 |
| TESTI2008020 | 0 | 0 | 0 | 0 | 0 | 0 | 67.687 |
| TESTI2008032 | 0 | 0 | 0 | 0 | 0 | 0 | 100 |
| TESTI2008033 | 0 | 0 | 0 | 0 | 0 | 0 | 100 |
| TESTI2008046 | 0 | 0 | 0 | 0 | 0 | 0 | 100 |
| TESTI2008081 | 0 | 0 | 0 | 0 | 0 | 0 | 100 |
| TESTI2008099 | 0 | 0 | 0 | 0 | 0 | 0 | 100 |
| TESTI2008139 | 0 | 0 | 0 | 0 | 0 | 0 | 100 |
| TESTI2008144 | 0 | 0 | 0 | 0 | 0 | 0 | 100 |
| TESTI2008161 | 0 | 0 | 0 | 0 | 0 | 0 | 100 |
| TESTI2008189 | 0 | 0 | 0 | 0 | 0 | 0 | 100 |
| TESTI2008204 | 0 | 0 | 0 | 0 | 0 | 0 | 100 |
| TESTI2008219 | 0 | 0 | 0 | 0 | 0 | 0 | 100 |
| TESTI2008233 | 28.364 | 0 | 0 | 0 | 0 | 0 | 25.789 |
| TESTI2008234 | 0 | 0 | 0 | 0 | 0 | 0 | 100 |
| TESTI2008237 | 0 | 0 | 0 | 0 | 0 | 0 | 100 |
| TESTI2008240 | 0.539 | 0 | 0 | 0 | 0 | 0 | 1.798 |
| TESTI2008278 | 0 | 0 | 0 | 0 | 0 | 0 | 56.337 |
| TESTI2008320 | 0 | 0 | 0 | 0 | 0 | 0 | 100 |
| TESTI2008331 | 0 | 0 | 0 | 0 | 0 | 0 | 100 |
| TESTI2008343 | 0 | 0 | 0 | 0 | 0 | 0 | 100 |
| TESTI2008387 | 0 | 0 | 0 | 0 | 0 | 0 | 100 |
| TESTI2008389 | 0 | 0 | 0 | 0 | 0 | 0 | 100 |
| TESTI2008394 | 0 | 0 | 0 | 0 | 0 | 0 | 26.135 |
| TESTI2008405 | 0 | 0 | 0 | 0 | 0 | 0 | 3.404 |
| TESTI2008420 | 0 | 0 | 0 | 0 | 0 | 0 | 100 |
| TESTI2008425 | 0 | 0 | 0 | 0 | 0 | 0 | 100 |
| TESTI2008440 | 0 | 0 | 0 | 0 | 0 | 0 | 100 |
| TESTI2008567 | 0 | 0 | 0 | 0 | 0 | 0 | 100 |
| TESTI2008595 | 0 | 0 | 0 | 0 | 0 | 0 | 100 |
| TESTI2008621 | 0 | 0 | 0 | 0 | 0 | 0 | 46.229 |
| TESTI2008636 | 0 | 0 | 0 | 0 | 0 | 0 | 100 |
| TESTI2008657 | 0 | 0 | 0 | 0 | 0 | 0 | 100 |
| TESTI2008684 | 0 | 0 | 0 | 0 | 0 | 0 | 100 |
| TESTI2008699 | 21.003 | 0 | 0 | 0 | 0 | 0 | 12.731 |
| TESTI2008715 | 0 | 0 | 0 | 0 | 0 | 0 | 100 |
| TESTI2008762 | 0 | 0 | 0 | 0 | 0 | 0 | 100 |
| TESTI2008774 | 0 | 0 | 0 | 0 | 0 | 0 | 100 |
| TESTI2008786 | 0 | 0 | 0 | 0 | 0 | 0 | 100 |
| TESTI2008822 | 0 | 0 | 0 | 0 | 0 | 0 | 100 |
| TESTI2008835 | 0 | 0 | 0 | 0 | 0 | 0 | 7.093 |
| TESTI2008840 | 0 | 0 | 0 | 0 | 0 | 0 | 27.035 |
| TESTI2008847 | 0 | 0 | 0 | 0 | 0 | 0 | 56.475 |
| TESTI2008884 | 0 | 0 | 0 | 0 | 0 | 0 | 100 |
| TESTI2008901 | 0 | 0 | 0 | 0 | 0 | 0 | 100 |
| TESTI2008929 | 0 | 0 | 0 | 0 | 0 | 0 | 100 |
| TESTI2008946 | 0 | 0 | 0 | 0 | 0 | 0 | 100 |
| TESTI2008960 | 0 | 0 | 0 | 0 | 0 | 0 | 100 |
| TESTI2009018 | 0 | 0 | 0 | 0 | 0 | 0 | 100 |
| TESTI2009091 | 0 | 0 | 0 | 0 | 0 | 0 | 100 |
| TESTI2009097 | 0 | 0 | 0 | 0 | 0 | 0 | 100 |
| TESTI2009136 | 0 | 0 | 0 | 0 | 0 | 0 | 100 |
| TESTI2009185 | 0 | 0 | 0 | 0 | 0 | 0 | 100 |
| TESTI2009190 | 0 | 0 | 0 | 0 | 0 | 0 | 100 |
| TESTI2009217 | 0 | 0 | 0 | 0 | 0 | 0 | 100 |
| TESTI2009239 | 0 | 0 | 0 | 0 | 0 | 0 | 47.943 |
| TESTI2009325 | 0 | 0 | 0 | 0 | 0 | 0 | 100 |
| TESTI2009388 | 0 | 0 | 0 | 0 | 0 | 0 | 13.106 |
| TESTI2009390 | 0 | 0 | 0 | 0 | 0 | 0 | 100 |
| TESTI2009400 | 0 | 0 | 0 | 0 | 0 | 0 | 100 |
| TESTI2009402 | 0 | 0 | 0 | 0 | 0 | 0 | 5.993 |
| TESTI2009412 | 0 | 0 | 0 | 0 | 0 | 0 | 100 |
| TESTI2009423 | 0 | 0 | 0 | 0 | 0 | 0 | 10.571 |
| TESTI2009447 | 0 | 0 | 0 | 0 | 0 | 0 | 100 |
| TESTI2009462 | 0 | 0 | 0 | 0 | 0 | 0 | 100 |
| TESTI2009474 | 0 | 44.875 | 0 | 0 | 0 | 0 | 2.641 |
| TESTI2009477 | 0 | 0 | 0 | 0 | 0 | 0 | 100 |
| TESTI2009497 | 0 | 0 | 0 | 0 | 0 | 0 | 34.478 |
| TESTI2009502 | 0 | 0 | 0 | 0 | 0 | 0 | 2.954 |
| TESTI2009511 | 0 | 0 | 0 | 0 | 0 | 0 | 100 |
| TESTI2009520 | 0 | 0 | 0 | 0 | 0 | 0 | 6.863 |
| TESTI2009544 | 0 | 0 | 0 | 0 | 0 | 0 | 100 |
| TESTI2009545 | 0 | 0 | 0 | 0 | 0 | 0 | 100 |
| TESTI2009551 | 0 | 0 | 0 | 0 | 0 | 0 | 100 |
| TESTI2009577 | 0 | 0 | 0 | 0 | 0 | 0 | 5.777 |
| TESTI2009585 | 0 | 0 | 0 | 0 | 0 | 0 | 100 |
| TESTI2009588 | 0 | 0 | 0 | 0 | 0 | 0 | 100 |
| TESTI2009683 | 0 | 0 | 0 | 0 | 0 | 0 | 39.348 |
| TESTI2009727 | 0 | 0 | 0 | 0 | 0 | 0 | 100 |
| TESTI2009739 | 0 | 0 | 0 | 0 | 0 | 0 | 100 |
| TESTI2009785 | 0 | 0 | 10.093 | 0 | 0 | 0 | 0.938 |
| TESTI2009812 | 0 | 0 | 0 | 0 | 0 | 0 | 100 |
| TESTI2009835 | 0 | 0 | 0 | 0 | 0 | 0 | 100 |
| TESTI2009853 | 0 | 0 | 0 | 0 | 0 | 0 | 100 |
| TESTI2009935 | 0 | 0 | 0 | 0 | 0 | 0 | 100 |
| TESTI2009977 | 0 | 0 | 0 | 0 | 0 | 0 | 15.790 |
| TESTI2009987 | 0 | 0 | 0 | 0 | 0 | 0 | 10.003 |
| TESTI2010009 | 0 | 0 | 0 | 0 | 0 | 0 | 100 |
| TESTI2010154 | 0 | 0 | 0 | 0 | 0 | 0 | 100 |
| TESTI2010180 | 0 | 0 | 0 | 0 | 0 | 0 | 22.675 |
| TESTI2010239 | 0 | 0 | 0 | 0 | 0 | 0 | 100 |
| TESTI2010280 | 0 | 0 | 0 | 0 | 0 | 0 | 100 |
| TESTI2010354 | 0 | 0 | 0 | 0 | 0 | 0 | 100 |
| TESTI2010396 | 0 | 0 | 0 | 0 | 0 | 0 | 100 |
| TESTI2010400 | 0 | 0 | 0 | 0 | 0 | 0 | 100 |
| TESTI2010409 | 0 | 0 | 0 | 0 | 0 | 0 | 100 |
| TESTI2010513 | 0 | 0 | 0 | 0 | 0 | 0 | 5.136 |
| TESTI2010572 | 0 | 0 | 0 | 0 | 0 | 0 | 100 |
| TESTI2010587 | 0 | 0 | 0 | 0 | 0 | 0 | 100 |
| TESTI2010591 | 0 | 0 | 0 | 0 | 0 | 0 | 100 |
| TESTI2010617 | 0 | 0 | 0 | 0 | 0 | 0 | 4.552 |
| TESTI2010682 | 0 | 0 | 0 | 0 | 0 | 0 | 100 |
| TESTI2010724 | 0 | 0 | 0 | 0 | 0 | 0 | 93.183 |
| TESTI2010732 | 0 | 0 | 0 | 0 | 0 | 0 | 100 |
| TESTI2010734 | 0 | 0 | 0 | 0 | 0 | 0 | 100 |
| TESTI2010755 | 0 | 0 | 0 | 0 | 0 | 0 | 100 |
| TESTI2010793 | 0 | 0 | 0 | 0 | 0 | 0 | 100 |
| TESTI2010806 | 0 | 0 | 0 | 0 | 0 | 0 | 100 |
| TESTI2010833 | 0 | 0 | 0 | 0 | 0 | 0 | 100 |
| TESTI2010872 | 0 | 0 | 0 | 0 | 0 | 0 | 100 |
| TESTI2011009 | 0 | 0 | 0 | 0 | 0 | 0 | 100 |
| TESTI2011020 | 0 | 0 | 0 | 0 | 0 | 0 | 100 |
| TESTI2011028 | 0 | 0 | 0 | 0 | 0 | 0 | 100 |
| TESTI2011033 | 0 | 0 | 0 | 0 | 0 | 0 | 100 |
| TESTI2011254 | 0 | 0 | 0 | 0 | 0 | 0 | 100 |
| TESTI2011286 | 0 | 0 | 0 | 0 | 0 | 0 | 17.123 |
| TESTI2011294 | 0 | 0 | 0 | 0 | 0 | 0 | 100 |
| TESTI2011314 | 0 | 0 | 0 | 0 | 0 | 0 | 100 |
| TESTI2011315 | 0 | 0 | 0 | 0 | 0 | 0 | 100 |
| TESTI2011394 | 0 | 0 | 0 | 0 | 0 | 0 | 100 |
| TESTI2011407 | 0 | 0 | 0 | 0 | 0 | 0 | 100 |
| TESTI2011448 | 0 | 0 | 0 | 0 | 0 | 0 | 100 |
| TESTI2011480 | 0 | 0 | 0 | 0 | 0 | 0 | 100 |
| TESTI2011549 | 0 | 0 | 0 | 0 | 0 | 0 | 39.348 |
| TESTI2011575 | 0 | 0 | 0 | 0 | 0 | 0 | 100 |
| TESTI2011605 | 0 | 0 | 0 | 0 | 0 | 0 | 25.917 |
| TESTI2011612 | 0 | 0 | 0 | 0 | 0 | 0 | 100 |
| TESTI2011665 | 0 | 0 | 0 | 0 | 0 | 0 | 100 |
| TESTI2011680 | 0 | 0 | 0 | 0 | 0 | 0 | 14.670 |
| TESTI2011683 | 0 | 0 | 0 | 0 | 0 | 0 | 21.164 |
| TESTI2011712 | 0 | 0 | 0 | 0 | 0 | 0 | 27.033 |
| TESTI2011750 | 0 | 0 | 0 | 0 | 0 | 0 | 100 |
| TESTI2011840 | 0 | 0 | 0 | 0 | 0 | 0 | 100 |
| TESTI2011846 | 0 | 0 | 0 | 0 | 0 | 0 | 100 |
| TESTI2011915 | 0 | 0 | 0 | 0 | 0 | 0 | 100 |
| TESTI2011928 | 0 | 0 | 0 | 0 | 0 | 0 | 100 |
| TESTI2011971 | 0 | 0 | 0 | 0 | 0 | 0 | 100 |
| TESTI2012019 | 0 | 0 | 0 | 0 | 0 | 0 | 7.602 |
| TESTI2012050 | 0 | 0 | 0 | 0 | 0 | 0 | 100 |
| TESTI2012090 | 0 | 0 | 0 | 0 | 0 | 0 | 100 |
| TESTI2012095 | 0 | 0 | 0 | 0 | 0 | 0 | 100 |
| TESTI2012104 | 0 | 0 | 0 | 0 | 0 | 0 | 6.364 |
| TESTI2012152 | 0 | 0 | 0 | 0 | 0 | 0 | 100 |
| TESTI2012155 | 0 | 0 | 0 | 0 | 0 | 0 | 8.378 |
| TESTI2012171 | 0 | 0 | 0 | 0 | 0 | 0 | 100 |
| TESTI2012207 | 0 | 0 | 0 | 0 | 0 | 0 | 100 |
| TESTI2012231 | 0 | 0 | 0 | 0 | 0 | 0 | 100 |
| TESTI2012244 | 0 | 0 | 0 | 0 | 0 | 0 | 100 |
| TESTI2012289 | 0 | 0 | 0 | 0 | 0 | 0 | 5.642 |
| TESTI2012308 | 0 | 0 | 0 | 0 | 0 | 0 | 100 |
| TESTI2012356 | 0 | 0 | 0 | 0 | 0 | 0 | 1.895 |
| TESTI2012387 | 0 | 0 | 0 | 0 | 0 | 0 | 100 |
| TESTI2012394 | 0 | 0 | 0 | 0 | 0 | 0 | 70.009 |
| TESTI2012444 | 0 | 0 | 0 | 0 | 0 | 0 | 100 |
| TESTI2012511 | 0 | 0 | 0 | 0 | 0 | 0 | 2.806 |
| TESTI2012528 | 0 | 0 | 0 | 0 | 0 | 0 | 100 |
| TESTI2012544 | 0 | 0 | 0 | 0 | 0 | 0 | 100 |
| TESTI2012550 | 0 | 0 | 0 | 0 | 0 | 0 | 100 |
| TESTI2012571 | 0 | 0 | 0 | 0 | 0 | 0 | 26.782 |
| TESTI2012592 | 0 | 0 | 0 | 0 | 0 | 0 | 100 |
| TESTI2012617 | 0 | 0 | 0 | 0 | 0 | 0 | 100 |
| TESTI2012628 | 0 | 0 | 0 | 0 | 0 | 0 | 100 |
| TESTI2012778 | 0 | 4.104 | 0 | 0 | 0 | 0 | 0.845 |
| TESTI2012812 | 0 | 0 | 0 | 0 | 0 | 0 | 100 |
| TESTI2012834 | 0 | 0 | 0 | 0 | 0 | 0 | 100 |
| TESTI2012835 | 0 | 0 | 0 | 0 | 0 | 0 | 100 |
| TESTI2012861 | 0 | 0 | 0 | 0 | 0 | 0 | 100 |
| TESTI2012900 | 0 | 0 | 0 | 0 | 0 | 0 | 100 |
| TESTI2012915 | 0 | 0 | 0 | 0 | 0 | 0 | 100 |
| TESTI2012922 | 0 | 0 | 0 | 0 | 0 | 0 | 100 |
| TESTI2013001 | 0 | 0 | 0 | 0 | 0 | 0 | 100 |
| TESTI2013012 | 0 | 0 | 0 | 0 | 0 | 0 | 100 |
| TESTI2013053 | 0 | 0 | 0 | 0 | 0 | 0 | 100 |
| TESTI2013144 | 0 | 0 | 0 | 0 | 0 | 0 | 100 |
| TESTI2013163 | 0 | 0 | 0 | 0 | 0 | 0 | 100 |
| TESTI2013169 | 0 | 0 | 0 | 0 | 0 | 0 | 100 |
| TESTI2013212 | 0 | 0 | 0 | 0 | 0 | 0 | 100 |
| TESTI2013231 | 0 | 0 | 0 | 0 | 0 | 0 | 1.301 |
| TESTI2013257 | 0 | 0 | 0 | 0 | 0 | 0 | 100 |
| TESTI2013264 | 0 | 0 | 0 | 0 | 0 | 0 | 16.423 |
| TESTI2013268 | 0 | 0 | 0 | 0 | 0 | 0 | 100 |
| TESTI2013295 | 0 | 0 | 0 | 0 | 0 | 0 | 6.964 |
| TESTI2013322 | 0 | 0 | 0 | 0 | 0 | 0 | 100 |
| TESTI2013381 | 0 | 0 | 0 | 0 | 0 | 0 | 100 |
| TESTI2013382 | 0 | 0 | 0 | 0 | 0 | 0 | 100 |
| TESTI2013401 | 0 | 0 | 0 | 0 | 0 | 0 | 100 |
| TESTI2013427 | 0 | 0 | 0 | 0 | 0 | 0 | 100 |
| TESTI2013468 | 0 | 0 | 0 | 0 | 0 | 0 | 100 |
| TESTI2013497 | 0 | 0 | 0 | 0 | 0 | 0 | 100 |
| TESTI2013545 | 0 | 0 | 0 | 0 | 0 | 0 | 100 |
| TESTI2013546 | 0 | 0 | 0 | 0 | 0 | 0 | 100 |
| TESTI2013566 | 0 | 0 | 0 | 0 | 0 | 0 | 100 |
| TESTI2013601 | 0 | 0 | 0 | 0 | 0 | 0 | 100 |
| TESTI2013604 | 0 | 0 | 0 | 0 | 0 | 0 | 100 |
| TESTI2013607 | 0 | 0 | 0 | 0 | 0 | 0 | 100 |
| TESTI2013610 | 0 | 0 | 0 | 0 | 0 | 0 | 100 |
| TESTI2013691 | 0 | 0 | 0 | 0 | 0 | 0 | 100 |
| TESTI2013699 | 0 | 0 | 0 | 0 | 0 | 0 | 100 |
| TESTI2013737 | 0 | 0 | 0 | 0 | 0 | 0 | 100 |
| TESTI2013767 | 0 | 0 | 0 | 0 | 0 | 0 | 100 |
| TESTI2013832 | 0 | 0 | 0 | 0 | 0 | 0 | 100 |
| TESTI2013873 | 0 | 0 | 0 | 0 | 0 | 0 | 100 |
| TESTI2013889 | 0 | 0 | 0 | 0 | 0 | 0 | 100 |
| TESTI2014007 | 0 | 0 | 0 | 0 | 0 | 0 | 100 |
| TESTI2014036 | 0 | 0 | 0 | 0 | 0 | 0 | 100 |
| TESTI2014097 | 0 | 0 | 0 | 0 | 0 | 0 | 100 |
| TESTI2014160 | 0 | 0 | 0 | 0 | 0 | 0 | 100 |
| TESTI2014248 | 0 | 0 | 0 | 0 | 0 | 0 | 100 |
| TESTI2014254 | 0 | 0 | 0 | 0 | 0 | 0 | 100 |
| TESTI2014269 | 0 | 0 | 0 | 0 | 0 | 0 | 100 |
| TESTI2014274 | 0 | 0 | 0 | 0 | 0 | 0 | 100 |
| TESTI2014318 | 0 | 0 | 0 | 0 | 0 | 0 | 18.792 |
| TESTI2014324 | 0 | 0 | 0 | 0 | 0 | 0 | 100 |
| TESTI2014339 | 0 | 0 | 0 | 0 | 0 | 0 | 100 |
| TESTI2014362 | 0 | 0 | 0 | 0 | 0 | 0 | 100 |
| TESTI2014385 | 0 | 0 | 0 | 0 | 0 | 0 | 100 |
| TESTI2014415 | 0 | 0 | 0 | 0 | 0 | 0 | 100 |
| TESTI2014439 | 0 | 0 | 0 | 0 | 0 | 0 | 100 |
| TESTI2014474 | 0 | 0 | 0 | 0 | 0 | 0 | 100 |
| TESTI2014505 | 0 | 0 | 0 | 0 | 0 | 0 | 12.881 |
| TESTI2014577 | 0 | 0 | 0 | 0 | 0 | 0 | 100 |
| TESTI2014578 | 0 | 0 | 0 | 0 | 0 | 0 | 100 |
| TESTI2014716 | 0 | 0 | 0 | 0 | 0 | 0 | 100 |
| TESTI2014780 | 0 | 0 | 0 | 0 | 0 | 0 | 18.563 |
| TESTI2014800 | 0 | 0 | 0 | 0 | 0 | 0 | 100 |
| TESTI2014838 | 0 | 0 | 0 | 0 | 0 | 0 | 77.083 |
| TESTI2014843 | 0 | 0 | 0 | 0 | 0 | 0 | 100 |
| TESTI2014979 | 0 | 0 | 0 | 0 | 0 | 0 | 100 |
| TESTI2014988 | 0 | 0 | 0 | 0 | 0 | 0 | 100 |
| TESTI2015042 | 0 | 0 | 0 | 0 | 0 | 0 | 56.168 |
| TESTI2015092 | 0 | 0 | 0 | 0 | 0 | 0 | 4.331 |
| TESTI2015105 | 0 | 0 | 0 | 0 | 0 | 0 | 100 |
| TESTI2015180 | 0 | 0 | 0 | 0 | 0 | 0 | 100 |
| TESTI2015213 | 0 | 0 | 3.610 | 0 | 0 | 0 | 1.342 |
| TESTI2015246 | 25.535 | 0 | 0 | 0 | 0 | 0 | 15.478 |
| TESTI2015249 | 0 | 0 | 0 | 0 | 0 | 0 | 100 |
| TESTI2015327 | 0 | 0 | 0 | 0 | 0 | 0 | 100 |
| TESTI2015331 | 0 | 0 | 0 | 0 | 0 | 0 | 100 |
| TESTI2015335 | 0 | 0 | 0 | 0 | 0 | 0 | 44.030 |
| TESTI2015350 | 0 | 0 | 0 | 0 | 0 | 0 | 100 |
| TESTI2015375 | 0 | 0 | 0 | 0 | 0 | 0 | 100 |
| TESTI2015437 | 0 | 0 | 0 | 0 | 0 | 0 | 2.669 |
| TESTI2015590 | 0 | 0 | 0 | 0 | 0 | 0 | 100 |
| TESTI2015610 | 0 | 0 | 0 | 0 | 0 | 0 | 100 |
| TESTI2015626 | 0 | 7.305 | 0 | 0 | 0 | 2.250 | 1.720 |
| TESTI2015647 | 0 | 0 | 0 | 0 | 0 | 0 | 100 |
| TESTI2015675 | 0 | 0 | 0 | 0 | 0 | 0 | 100 |
| TESTI2015710 | 0 | 0 | 0 | 0 | 0 | 0 | 100 |
| TESTI2015752 | 0 | 0 | 0 | 0 | 0 | 0 | 100 |
| TESTI2015830 | 0 | 0 | 0 | 0 | 0 | 0 | 100 |
| TESTI2015914 | 0 | 0 | 0 | 0 | 0 | 0 | 100 |
| TESTI2015938 | 0 | 0 | 0 | 0 | 0 | 0 | 100 |
| TESTI2015947 | 0 | 0 | 0 | 0 | 0 | 0 | 100 |
| TESTI2015962 | 0 | 0 | 0 | 0 | 0 | 0 | 2.593 |
| TESTI2015987 | 0 | 0 | 0 | 0 | 0 | 0 | 100 |
| TESTI2016033 | 0 | 0 | 0 | 0 | 0 | 0 | 2.278 |
| TESTI2016046 | 0 | 0 | 0 | 0 | 0 | 0 | 100 |
| TESTI2016101 | 0 | 0 | 0 | 0 | 0 | 0 | 5.668 |
| TESTI2016162 | 0 | 0 | 0 | 0 | 0 | 0 | 100 |
| TESTI2016197 | 0 | 0 | 0 | 0 | 0 | 0 | 100 |
| TESTI2016278 | 0 | 0 | 0 | 0 | 0 | 0 | 37.085 |
| TESTI2016298 | 0 | 0 | 0 | 0 | 0 | 0 | 100 |
| TESTI2016331 | 0 | 0 | 0 | 0 | 0 | 0 | 100 |
| TESTI2016337 | 0 | 0 | 0 | 0 | 0 | 0 | 100 |
| TESTI2016410 | 0 | 0 | 0 | 0 | 0 | 0 | 100 |
| TESTI2016421 | 0 | 0 | 0 | 0 | 0 | 0 | 100 |
| TESTI2016509 | 0 | 0 | 0 | 0 | 0 | 0 | 100 |
| TESTI2016568 | 0 | 0 | 0 | 0 | 0 | 0 | 100 |
| TESTI2016583 | 0 | 0 | 0 | 0 | 0 | 0 | 23.515 |
| TESTI2016599 | 0 | 0 | 0 | 0 | 0 | 0 | 100 |
| TESTI2016629 | 0 | 0 | 0 | 0 | 0 | 0 | 100 |
| TESTI2016663 | 0 | 0 | 0 | 0 | 0 | 0 | 100 |
| TESTI2016667 | 0 | 0 | 0 | 0 | 0 | 0 | 100 |
| TESTI2016688 | 0 | 0 | 0 | 0 | 0 | 0 | 100 |
| TESTI2016701 | 0 | 0 | 0 | 0 | 0 | 0 | 47.943 |
| TESTI2016758 | 0 | 0 | 0 | 0 | 0 | 0 | 100 |
| TESTI2016848 | 0 | 0 | 0 | 0 | 0 | 0 | 100 |
| TESTI2016863 | 0 | 0 | 0 | 0 | 0 | 0 | 100 |
| TESTI2016888 | 0 | 0 | 0 | 0 | 0 | 0 | 100 |
| TESTI2016896 | 0 | 0 | 0 | 0 | 0 | 0 | 56.168 |
| TESTI2016922 | 0 | 0 | 0 | 0 | 0 | 0 | 100 |
| TESTI2016942 | 0 | 0 | 0 | 0 | 0 | 0 | 100 |
| TESTI2016950 | 0 | 0 | 0 | 0 | 0 | 0 | 100 |
| TESTI2016996 | 0 | 0 | 0 | 0 | 0 | 0 | 100 |
| TESTI2017017 | 0 | 0 | 0 | 0 | 0 | 0 | 100 |
| TESTI2017028 | 0 | 0 | 0 | 0 | 0 | 0 | 100 |
| TESTI2017035 | 0 | 0 | 0 | 0 | 0 | 0 | 27.423 |
| TESTI2017069 | 0 | 0 | 0 | 0 | 0 | 0 | 100 |
| TESTI2017089 | 0 | 0 | 0 | 0 | 0 | 0 | 100 |
| TESTI2017102 | 0 | 0 | 0 | 0 | 0 | 0 | 100 |
| TESTI2017107 | 0 | 0 | 0 | 0 | 0 | 0 | 39.348 |
| TESTI2017113 | 0 | 0 | 0 | 0 | 0 | 0 | 100 |
| TESTI2017125 | 0 | 0 | 0 | 0 | 0 | 0 | 100 |
| TESTI2017256 | 0 | 0 | 0 | 0 | 0 | 0 | 100 |
| TESTI2017305 | 0 | 0 | 0 | 0 | 0 | 0 | 100 |
| TESTI2017311 | 0 | 0 | 0 | 0 | 0 | 0 | 100 |
| TESTI2017396 | 0 | 0 | 0 | 0 | 0 | 0 | 100 |
| TESTI2017507 | 0 | 0 | 0 | 0 | 0 | 0 | 100 |
| TESTI2017537 | 0 | 0 | 0 | 0 | 0 | 0 | 100 |
| TESTI2017582 | 0 | 0 | 0 | 0 | 0 | 0 | 100 |
| TESTI2017584 | 0 | 0 | 0 | 0 | 0 | 0 | 100 |
| TESTI2017645 | 0 | 0 | 0 | 0 | 0 | 0 | 21.754 |
| TESTI2017727 | 0 | 0 | 0 | 0 | 0 | 0 | 100 |
| TESTI2017740 | 0 | 0 | 0 | 0 | 0 | 0 | 100 |
| TESTI2017775 | 0 | 0 | 0 | 0 | 0 | 0 | 100 |
| TESTI2017816 | 0 | 0 | 0 | 0 | 0 | 0 | 100 |
| TESTI2017832 | 0 | 0 | 0 | 0 | 0 | 0 | 100 |
| TESTI2017923 | 0 | 0 | 0 | 0 | 0 | 0 | 100 |
| TESTI2017932 | 0 | 0 | 0 | 0 | 0 | 0 | 100 |
| TESTI2017951 | 0 | 0 | 0 | 0 | 0 | 0 | 100 |
| TESTI2017954 | 0 | 14.714 | 0 | 0 | 0 | 0 | 0.866 |
| TESTI2018014 | 0 | 0 | 0 | 0 | 0 | 0 | 5.039 |
| TESTI2018031 | 0 | 0 | 0 | 0 | 0 | 0 | 100 |
| TESTI2018035 | 0 | 0 | 0 | 0 | 0 | 0 | 100 |
| TESTI2018060 | 0 | 0 | 0 | 0 | 0 | 0 | 100 |
| TESTI2018083 | 0 | 0 | 0 | 0 | 0 | 0 | 100 |
| TESTI2018178 | 0 | 0 | 0 | 0 | 0 | 0 | 100 |
| TESTI2018221 | 0 | 0 | 0 | 0 | 0 | 0 | 100 |
| TESTI2018276 | 0 | 0 | 0 | 0 | 0 | 0 | 100 |
| TESTI2018290 | 0 | 0 | 0 | 0 | 0 | 0 | 100 |
| TESTI2018335 | 0 | 0 | 0 | 0 | 0 | 0 | 100 |
| TESTI2018337 | 0 | 0 | 0 | 0 | 0 | 0 | 100 |
| TESTI2018368 | 0 | 0 | 0 | 0 | 0 | 0 | 100 |
| TESTI2018421 | 0 | 0 | 0 | 0 | 0 | 0 | 15.347 |
| TESTI2018428 | 0 | 0 | 0 | 0 | 0 | 0 | 100 |
| TESTI2018462 | 6.095 | 0 | 0 | 0 | 0 | 0 | 9.236 |
| TESTI2018475 | 0 | 0 | 0 | 0 | 0 | 0 | 100 |
| TESTI2018482 | 0 | 0 | 0 | 0 | 0 | 0 | 100 |
| TESTI2018521 | 0 | 0 | 0 | 0 | 0 | 0 | 100 |
| TESTI2018565 | 0 | 0 | 0 | 0 | 0 | 0 | 100 |
| TESTI2018581 | 0 | 0 | 0 | 0 | 0 | 0 | 100 |
| TESTI2018611 | 0 | 0 | 0 | 0 | 0 | 0 | 3.433 |
| TESTI2018687 | 0 | 0 | 0 | 0 | 0 | 0 | 100 |
| TESTI2018697 | 0 | 0 | 0 | 0 | 0 | 0 | 100 |
| TESTI2018838 | 0 | 0 | 0 | 0 | 0 | 0 | 100 |
| TESTI2018867 | 0 | 0 | 0 | 0 | 0 | 0 | 72.596 |
| TESTI2018941 | 0 | 0 | 0 | 0 | 0 | 0 | 100 |
| TESTI2018957 | 0 | 0 | 0 | 0 | 0 | 0 | 100 |
| TESTI2018974 | 0 | 0 | 0 | 0 | 0 | 0 | 100 |
| TESTI2018975 | 0 | 0 | 0 | 0 | 0 | 0 | 100 |
| TESTI2018997 | 0 | 0 | 14.651 | 0 | 0 | 0 | 2.723 |
| TESTI2019042 | 0 | 0 | 0 | 0 | 0 | 0 | 100 |
| TESTI2019077 | 0 | 0 | 0 | 0 | 0 | 0 | 43.944 |
| TESTI2019228 | 0 | 0 | 0 | 0 | 0 | 0 | 100 |
| TESTI2019257 | 0 | 0 | 0 | 0 | 0 | 0 | 100 |
| TESTI2019280 | 0 | 0 | 0 | 0 | 0 | 0 | 100 |
| TESTI2019282 | 0 | 0 | 0 | 0 | 0 | 0 | 100 |
| TESTI2019308 | 0 | 0 | 0 | 0 | 0 | 0 | 100 |
| TESTI2019336 | 0 | 0 | 0 | 0 | 0 | 0 | 100 |
| TESTI2019352 | 0 | 0 | 0 | 0 | 0 | 0 | 100 |
| TESTI2019406 | 0 | 0 | 0 | 0 | 0 | 0 | 100 |
| TESTI2019430 | 0 | 0 | 0 | 0 | 0 | 0 | 100 |
| TESTI2019455 | 0 | 0 | 0 | 0 | 0 | 0 | 100 |
| TESTI2019481 | 0 | 0 | 0 | 0 | 0 | 0 | 100 |
| TESTI2019488 | 0 | 0 | 0 | 0 | 0 | 0 | 100 |
| TESTI2019501 | 0 | 0 | 0 | 0 | 0 | 0 | 100 |
| TESTI2019648 | 0 | 0 | 0 | 0 | 0 | 0 | 100 |
| TESTI2019669 | 0 | 0 | 0 | 0 | 0 | 0 | 100 |
| TESTI2019697 | 0 | 0 | 0 | 0 | 0 | 0 | 100 |
| TESTI2019729 | 0 | 0 | 0 | 0 | 0 | 0 | 25.979 |
| TESTI2019760 | 0 | 0 | 0 | 0 | 0 | 0 | 29.749 |
| TESTI2019787 | 0 | 0 | 0 | 0 | 0 | 0 | 100 |
| TESTI2019794 | 0 | 0 | 0 | 0 | 0 | 0 | 100 |
| TESTI2019854 | 0 | 0 | 0 | 0 | 0 | 0 | 100 |
| TESTI2019859 | 0 | 0 | 0 | 0 | 0 | 0 | 100 |
| TESTI2019860 | 0 | 0 | 0 | 0 | 0 | 0 | 100 |
| TESTI2019872 | 0 | 0 | 0 | 0 | 0 | 0 | 100 |
| TESTI2019911 | 0 | 0 | 0 | 0 | 0 | 0 | 100 |
| TESTI2019917 | 0 | 0 | 0 | 0 | 0 | 0 | 100 |
| TESTI2019975 | 0 | 0 | 0 | 0 | 0 | 0 | 100 |
| TESTI2019998 | 0 | 0 | 0 | 0 | 0 | 0 | 100 |
| TESTI2020012 | 0 | 0 | 0 | 0 | 0 | 0 | 14.939 |
| TESTI2020026 | 0 | 0 | 0 | 0 | 0 | 0 | 8.489 |
| TESTI2020029 | 0 | 0 | 0 | 0 | 0 | 0 | 100 |
| TESTI2020071 | 0 | 0 | 0 | 0 | 0 | 0 | 100 |
| TESTI2020084 | 0 | 0 | 0 | 0 | 0 | 0 | 100 |
| TESTI2020265 | 0 | 0 | 0 | 0 | 0 | 0 | 100 |
| TESTI2020283 | 0 | 0 | 0 | 0 | 0 | 0 | 100 |
| TESTI2020316 | 0 | 0 | 0 | 0 | 0 | 0 | 100 |
| TESTI2020344 | 0 | 0 | 0 | 0 | 0 | 0 | 100 |
| TESTI2020379 | 0 | 0 | 0 | 0 | 0 | 0 | 100 |
| TESTI2020426 | 0 | 0 | 0 | 0 | 0 | 0 | 100 |
| TESTI2020445 | 0 | 0 | 0 | 0 | 0 | 0 | 100 |
| TESTI2020510 | 0 | 0 | 0 | 0 | 0 | 0 | 4.728 |
| TESTI2020515 | 0 | 0 | 0 | 0 | 0 | 0 | 100 |
| TESTI2020525 | 0 | 0 | 0 | 0 | 0 | 0 | 21.336 |
| TESTI2020579 | 0 | 0 | 0 | 0 | 0 | 0 | 100 |
| TESTI2020717 | 0 | 0 | 0 | 0 | 0 | 0 | 100 |
| TESTI2020840 | 0 | 0 | 0 | 0 | 0 | 0 | 74.775 |
| TESTI2020871 | 0 | 0 | 0 | 0 | 0 | 0 | 35.404 |
| TESTI2020905 | 0 | 0 | 0 | 0 | 0 | 0 | 100 |
| TESTI2020906 | 0 | 0 | 0 | 0 | 0 | 0 | 100 |
| TESTI2020946 | 0 | 0 | 0 | 0 | 0 | 0 | 8.001 |
| TESTI2020956 | 0 | 0 | 0 | 0 | 0 | 0 | 2.813 |
| TESTI2020981 | 0 | 0 | 0 | 0 | 0 | 0 | 100 |
| TESTI2020999 | 0 | 0 | 0 | 0 | 0 | 0 | 100 |
| TESTI2021003 | 0 | 0 | 0 | 0 | 0 | 0 | 100 |
| TESTI2021038 | 0 | 0 | 0 | 0 | 0 | 0 | 27.658 |
| TESTI2021057 | 0 | 0 | 0 | 0 | 0 | 0 | 100 |
| TESTI2021077 | 0 | 0 | 0 | 0 | 0 | 0 | 100 |
| TESTI2021112 | 0 | 0 | 0 | 0 | 0 | 0 | 100 |
| TESTI2021114 | 0 | 0 | 0 | 0 | 0 | 0 | 100 |
| TESTI2021116 | 0 | 0 | 0 | 0 | 0 | 0 | 100 |
| TESTI2021118 | 0 | 0 | 0 | 0 | 0 | 0 | 7.012 |
| TESTI2021122 | 0 | 0 | 0 | 0 | 0 | 0 | 100 |
| TESTI2021124 | 0 | 0 | 0 | 0 | 0 | 0 | 3.736 |
| TESTI2021138 | 0 | 0 | 0 | 0 | 0 | 0 | 100 |
| TESTI2021297 | 0 | 0 | 0 | 0 | 0 | 0 | 4.488 |
| TESTI2021315 | 0 | 0 | 0 | 0 | 0 | 0 | 26.494 |
| TESTI2021358 | 0 | 0 | 0 | 0 | 0 | 0 | 22.119 |
| TESTI2021425 | 0 | 0 | 0 | 36.333 | 0 | 0 | 6.752 |
| TESTI2021447 | 0 | 0 | 0 | 0 | 0 | 0 | 67.792 |
| TESTI2021463 | 0 | 0 | 0 | 0 | 0 | 0 | 100 |
| TESTI2021531 | 0 | 0 | 0 | 0 | 0 | 0 | 10.937 |
| TESTI2021599 | 0 | 0 | 0 | 0 | 0 | 0 | 100 |
| TESTI2021637 | 0 | 0 | 0 | 0 | 0 | 0 | 100 |
| TESTI2021654 | 0 | 0 | 0 | 0 | 0 | 0 | 6.389 |
| TESTI2021667 | 0 | 0 | 0 | 0 | 0 | 0 | 100 |
| TESTI2021736 | 0 | 0 | 0 | 0 | 0 | 0 | 100 |
| TESTI2021785 | 0 | 0 | 0 | 0 | 0 | 0 | 100 |
| TESTI2021819 | 0 | 0 | 0 | 0 | 0 | 0 | 100 |
| TESTI2021851 | 0 | 0 | 0 | 0 | 0 | 0 | 12.368 |
| TESTI2021895 | 0 | 0 | 0 | 0 | 0 | 0 | 100 |
| TESTI2021911 | 0 | 0 | 0 | 0 | 0 | 0 | 100 |
| TESTI2021939 | 0 | 0 | 0 | 0 | 0 | 0 | 100 |
| TESTI2021999 | 0 | 0 | 0 | 0 | 0 | 0 | 100 |
| TESTI2022010 | 0 | 0 | 0 | 0 | 0 | 0 | 100 |
| TESTI2022023 | 0 | 0 | 0 | 0 | 0 | 0 | 100 |
| TESTI2022065 | 0 | 0 | 0 | 0 | 0 | 0 | 100 |
| TESTI2022086 | 0 | 0 | 0 | 0 | 0 | 0 | 100 |
| TESTI2022179 | 0 | 0 | 0 | 0 | 0 | 0 | 48.059 |
| TESTI2022203 | 0 | 0 | 0 | 0 | 0 | 0 | 100 |
| TESTI2022246 | 0 | 0 | 0 | 0 | 0 | 0 | 100 |
| TESTI2022323 | 0 | 0 | 0 | 0 | 0 | 0 | 100 |
| TESTI2022338 | 0 | 0 | 0 | 0 | 0 | 0 | 100 |
| TESTI2022400 | 0 | 0 | 0 | 0 | 0 | 0 | 100 |
| TESTI2022462 | 0 | 0 | 0 | 0 | 0 | 0 | 100 |
| TESTI2022495 | 0 | 0 | 0 | 0 | 0 | 0 | 100 |
| TESTI2022623 | 0 | 0 | 0 | 0 | 0 | 0 | 100 |
| TESTI2022649 | 0 | 0 | 0 | 0 | 0 | 0 | 100 |
| TESTI2022652 | 0 | 0 | 0 | 0 | 0 | 0 | 100 |
| TESTI2022738 | 0 | 0 | 0 | 0 | 0 | 0 | 100 |
| TESTI2022798 | 0 | 0 | 0 | 0 | 0 | 0 | 100 |
| TESTI2022812 | 0 | 0 | 0 | 0 | 0 | 0 | 100 |
| TESTI2022814 | 0 | 0 | 0 | 0 | 0 | 0 | 100 |
| TESTI2022874 | 0 | 0 | 0 | 0 | 0 | 0 | 100 |
| TESTI2022940 | 0 | 0 | 0 | 0 | 0 | 0 | 100 |
| TESTI2022952 | 0 | 0 | 0 | 0 | 0 | 0 | 100 |
| TESTI2023025 | 0 | 0 | 0 | 0 | 0 | 0 | 8.225 |
| TESTI2023033 | 0 | 0 | 0 | 0 | 0 | 0 | 35.902 |
| TESTI2023053 | 0 | 0 | 0 | 0 | 0 | 0 | 100 |
| TESTI2023085 | 0 | 0 | 0 | 0 | 0 | 0 | 100 |
| TESTI2023192 | 0 | 0 | 0 | 0 | 0 | 0 | 100 |
| TESTI2023194 | 0 | 0 | 0 | 0 | 0 | 0 | 100 |
| TESTI2023214 | 0 | 0 | 0 | 0 | 0 | 0 | 100 |
| TESTI2023254 | 0 | 0 | 0 | 0 | 0 | 0 | 100 |
| TESTI2023386 | 0 | 0 | 0 | 0 | 0 | 0 | 100 |
| TESTI2023414 | 0 | 0 | 0 | 0 | 0 | 0 | 2.661 |
| TESTI2023427 | 0 | 0 | 0 | 0 | 0 | 0 | 100 |
| TESTI2023436 | 0 | 0 | 0 | 0 | 0 | 0 | 100 |
| TESTI2023599 | 0 | 0 | 0 | 0 | 0 | 0 | 100 |
| TESTI2023752 | 0 | 0 | 0 | 0 | 0 | 0 | 100 |
| TESTI2023800 | 0 | 0 | 0 | 0 | 0 | 0 | 100 |
| TESTI2023861 | 0 | 0 | 0 | 0 | 0 | 0 | 100 |
| TESTI2023903 | 0 | 0 | 0 | 0 | 0 | 0 | 100 |
| TESTI2023942 | 0 | 0 | 0 | 0 | 0 | 0 | 100 |
| TESTI2023947 | 0 | 0 | 0 | 0 | 0 | 0 | 100 |
| TESTI2023951 | 0 | 0 | 0 | 0 | 0 | 0 | 100 |
| TESTI2023968 | 0 | 0 | 0 | 0 | 0 | 0 | 100 |
| TESTI2024009 | 0 | 0 | 0 | 0 | 0 | 0 | 100 |
| TESTI2024090 | 0 | 0 | 0 | 0 | 0 | 0 | 26.863 |
| TESTI2024125 | 76.742 | 0 | 0 | 0 | 0 | 0 | 23.258 |
| TESTI2024153 | 0 | 0 | 0 | 0 | 0 | 0 | 100 |
| TESTI2024192 | 0 | 0 | 0 | 0 | 0 | 0 | 100 |
| TESTI2024267 | 0 | 0 | 0 | 0 | 0 | 0 | 100 |
| TESTI2024283 | 0 | 0 | 0 | 0 | 0 | 0 | 36.852 |
| TESTI2024299 | 0 | 0 | 0 | 0 | 0 | 0 | 100 |
| TESTI2024419 | 0 | 0 | 0 | 0 | 0 | 0 | 100 |
| TESTI2024422 | 0 | 0 | 0 | 0 | 0 | 0 | 100 |
| TESTI2024443 | 0 | 0 | 0 | 0 | 0 | 0 | 100 |
| TESTI2024446 | 0 | 0 | 0 | 0 | 0 | 0 | 100 |
| TESTI2024473 | 0 | 0 | 0 | 0 | 0 | 0 | 100 |
| TESTI2024476 | 0 | 0 | 0 | 0 | 0 | 0 | 100 |
| TESTI2024498 | 0 | 0 | 0 | 0 | 0 | 0 | 100 |
| TESTI2024560 | 0 | 0 | 0 | 0 | 0 | 0 | 100 |
| TESTI2024567 | 0 | 0 | 0 | 0 | 0 | 0 | 100 |
| TESTI2024586 | 0 | 0 | 0 | 0 | 0 | 0 | 100 |
| TESTI2024598 | 0 | 0 | 0 | 0 | 0 | 0 | 39.051 |
| TESTI2024641 | 0 | 0 | 0 | 0 | 0 | 0 | 100 |
| TESTI2024648 | 0 | 0 | 0 | 0 | 0 | 0 | 100 |
| TESTI2024718 | 0 | 0 | 0 | 0 | 0 | 0 | 100 |
| TESTI2024744 | 0 | 0 | 0 | 0 | 0 | 0 | 100 |
| TESTI2024844 | 0 | 0 | 0 | 0 | 0 | 0 | 2.995 |
| TESTI2024864 | 0 | 0 | 0 | 0 | 0 | 0 | 18.051 |
| TESTI2024885 | 0 | 0 | 0 | 0 | 0 | 0 | 8.693 |
| TESTI2024936 | 0 | 0 | 0 | 0 | 0 | 0 | 100 |
| TESTI2024978 | 0 | 0 | 0 | 0 | 0 | 0 | 100 |
| TESTI2024999 | 0 | 0 | 0 | 0 | 0 | 0 | 100 |
| TESTI2025006 | 0 | 0 | 0 | 0 | 0 | 0 | 100 |
| TESTI2025013 | 0 | 0 | 0 | 0 | 0 | 0 | 2.327 |
| TESTI2025022 | 0 | 0 | 0 | 0 | 0 | 0 | 100 |
| TESTI2025063 | 0 | 0 | 0 | 0 | 0 | 0 | 100 |
| TESTI2025117 | 0 | 0 | 0 | 0 | 0 | 0 | 100 |
| TESTI2025144 | 0 | 0 | 0 | 0 | 0 | 0 | 100 |
| TESTI2025161 | 0 | 0 | 0 | 0 | 0 | 0 | 100 |
| TESTI2025174 | 0 | 0 | 0 | 0 | 0 | 0 | 100 |
| TESTI2025229 | 0 | 0 | 0 | 0 | 0 | 0 | 100 |
| TESTI2025269 | 0 | 0 | 0 | 0 | 0 | 0 | 100 |
| TESTI2025365 | 0 | 0 | 0 | 0 | 0 | 0 | 100 |
| TESTI2025403 | 0 | 0 | 0 | 0 | 0 | 0 | 100 |
| TESTI2025409 | 0 | 0 | 0 | 0 | 0 | 0 | 100 |
| TESTI2025422 | 0 | 0 | 0 | 0 | 0 | 0 | 1.622 |
| TESTI2025448 | 0 | 0 | 0 | 0 | 0 | 0 | 100 |
| TESTI2025454 | 0 | 0 | 0 | 0 | 0 | 0 | 7.363 |
| TESTI2025486 | 0 | 0 | 0 | 0 | 0 | 0 | 100 |
| TESTI2025546 | 0 | 0 | 0 | 0 | 0 | 0 | 68.062 |
| TESTI2025582 | 0 | 0 | 0 | 0 | 0 | 0 | 100 |
| TESTI2025609 | 0 | 0 | 0 | 0 | 0 | 0 | 100 |
| TESTI2025656 | 0 | 0 | 0 | 0 | 0 | 0 | 100 |
| TESTI2025757 | 0 | 0 | 0 | 0 | 0 | 0 | 11.084 |
| TESTI2025791 | 0 | 0 | 0 | 0 | 0 | 0 | 100 |
| TESTI2025846 | 0 | 0 | 0 | 0 | 0 | 0 | 13.307 |
| TESTI2025872 | 0 | 0 | 0 | 91.498 | 0 | 0 | 8.502 |
| TESTI2025879 | 0 | 0 | 0 | 0 | 0 | 0 | 100 |
| TESTI2025911 | 0 | 0 | 0 | 0 | 0 | 0 | 100 |
| TESTI2025920 | 0 | 0 | 0 | 0 | 0 | 0 | 100 |
| TESTI2025924 | 0 | 0 | 0 | 0 | 0 | 0 | 100 |
| TESTI2026014 | 0 | 0 | 0 | 0 | 0 | 0 | 100 |
| TESTI2026024 | 0 | 0 | 0 | 0 | 0 | 0 | 15.564 |
| TESTI2026064 | 0 | 0 | 0 | 0 | 0 | 0 | 100 |
| TESTI2026077 | 0 | 0 | 0 | 0 | 0 | 0 | 100 |
| TESTI2026104 | 0 | 0 | 0 | 0 | 0 | 0 | 100 |
| TESTI2026116 | 0 | 0 | 0 | 0 | 0 | 0 | 29.249 |
| TESTI2026134 | 0 | 0 | 0 | 0 | 0 | 0 | 100 |
| TESTI2026168 | 0 | 0 | 0 | 0 | 0 | 0 | 100 |
| TESTI2026196 | 0 | 0 | 0 | 0 | 0 | 0 | 100 |
| TESTI2026215 | 0 | 0 | 48.726 | 0 | 0 | 0 | 4.528 |
| TESTI2026218 | 0 | 0 | 0 | 0 | 0 | 0 | 100 |
| TESTI2026233 | 0 | 0 | 0 | 0 | 0 | 0 | 100 |
| TESTI2026291 | 0 | 0 | 0 | 0 | 0 | 0 | 100 |
| TESTI2026294 | 0 | 0 | 0 | 0 | 0 | 0 | 100 |
| TESTI2026401 | 0 | 0 | 0 | 0 | 0 | 0 | 100 |
| TESTI2026403 | 0 | 0 | 0 | 0 | 0 | 0 | 100 |
| TESTI2026453 | 0 | 0 | 0 | 0 | 0 | 0 | 100 |
| TESTI2026461 | 0 | 0 | 0 | 0 | 0 | 0 | 100 |
| TESTI2026491 | 0 | 0 | 0 | 0 | 0 | 0 | 100 |
| TESTI2026505 | 0 | 0 | 0 | 0 | 0 | 0 | 100 |
| TESTI2026515 | 0 | 0 | 0 | 0 | 0 | 0 | 100 |
| TESTI2026525 | 0 | 0 | 0 | 0 | 0 | 0 | 5.688 |
| TESTI2026534 | 0 | 0 | 0 | 0 | 0 | 0 | 39.051 |
| TESTI2026537 | 0 | 0 | 0 | 0 | 0 | 0 | 100 |
| TESTI2026589 | 0 | 0 | 0 | 0 | 0 | 0 | 100 |
| TESTI2026597 | 0 | 0 | 0 | 0 | 0 | 0 | 100 |
| TESTI2026605 | 0 | 0 | 0 | 0 | 0 | 0 | 100 |
| TESTI2026647 | 0 | 0 | 0 | 0 | 0 | 0 | 7.724 |
| TESTI2026674 | 0 | 0 | 0 | 0 | 0 | 0 | 100 |
| TESTI2026794 | 0 | 0 | 0 | 0 | 0 | 0 | 100 |
| TESTI2026824 | 0 | 0 | 0 | 0 | 0 | 0 | 100 |
| TESTI2026854 | 0 | 0 | 0 | 0 | 0 | 0 | 100 |
| TESTI2026925 | 0 | 0 | 0 | 0 | 0 | 0 | 100 |
| TESTI2026936 | 0 | 0 | 0 | 0 | 0 | 0 | 39.348 |
| TESTI2026957 | 0 | 0 | 0 | 0 | 0 | 0 | 100 |
| TESTI2027013 | 0 | 0 | 0 | 0 | 0 | 0 | 100 |
| TESTI2027019 | 0 | 0 | 0 | 0 | 0 | 0 | 100 |
| TESTI2027108 | 0 | 0 | 0 | 0 | 0 | 0 | 100 |
| TESTI2027165 | 0 | 0 | 0 | 0 | 0 | 0 | 35.404 |
| TESTI2027171 | 0 | 0 | 0 | 0 | 0 | 0 | 100 |
| TESTI2027173 | 0 | 0 | 0 | 0 | 0 | 0 | 100 |
| TESTI2027179 | 0 | 0 | 0 | 0 | 0 | 0 | 100 |
| TESTI2027185 | 0 | 0 | 0 | 0 | 0 | 0 | 100 |
| TESTI2027206 | 0 | 0 | 0 | 0 | 0 | 0 | 100 |
| TESTI2027238 | 0 | 0 | 0 | 0 | 0 | 0 | 100 |
| TESTI2027239 | 0 | 0 | 0 | 0 | 0 | 0 | 100 |
| TESTI2027243 | 0 | 0 | 0 | 0 | 0 | 0 | 100 |
| TESTI2027271 | 0 | 0 | 0 | 0 | 0 | 0 | 6.903 |
| TESTI2027296 | 0 | 0 | 0 | 0 | 0 | 0 | 100 |
| TESTI2027378 | 0 | 0 | 0 | 0 | 0 | 0 | 100 |
| TESTI2027411 | 0 | 0 | 0 | 0 | 0 | 0 | 100 |
| TESTI2027450 | 0 | 0 | 0 | 0 | 0 | 0 | 100 |
| TESTI2027496 | 0 | 0 | 0 | 0 | 0 | 0 | 12.283 |
| TESTI2027503 | 0 | 0 | 0 | 0 | 0 | 0 | 100 |
| TESTI2027562 | 0 | 0 | 0 | 0 | 0 | 0 | 100 |
| TESTI2027615 | 0 | 0 | 0 | 0 | 0 | 0 | 100 |
| TESTI2027645 | 0 | 0 | 0 | 0 | 0 | 0 | 100 |
| TESTI2027730 | 0 | 0 | 0 | 0 | 0 | 0 | 100 |
| TESTI2027736 | 0 | 0 | 0 | 0 | 0 | 0 | 100 |
| TESTI2027791 | 0 | 0 | 0 | 0 | 0 | 0 | 100 |
| TESTI2027820 | 0 | 0 | 0 | 0 | 0 | 0 | 100 |
| TESTI2027828 | 0 | 0 | 0 | 0 | 0 | 0 | 100 |
| TESTI2027840 | 0 | 0 | 0 | 0 | 0 | 0 | 100 |
| TESTI2027909 | 0 | 0 | 0 | 0 | 0 | 0 | 43.944 |
| TESTI2028098 | 0 | 0 | 0 | 0 | 0 | 0 | 100 |
| TESTI2028151 | 0 | 0 | 0 | 0 | 0 | 0 | 100 |
| TESTI2028242 | 0 | 0 | 0 | 0 | 0 | 0 | 100 |
| TESTI2028253 | 0 | 0 | 0 | 0 | 0 | 0 | 100 |
| TESTI2028254 | 0 | 0 | 0 | 0 | 0 | 0 | 100 |
| TESTI2028269 | 0 | 0 | 0 | 0 | 0 | 0 | 100 |
| TESTI2028290 | 0 | 0 | 0 | 0 | 0 | 0 | 100 |
| TESTI2028296 | 0 | 0 | 0 | 0 | 0 | 0 | 1.612 |
| TESTI2028384 | 0 | 0 | 0 | 0 | 0 | 0 | 100 |
| TESTI2028426 | 0 | 0 | 0 | 0 | 0 | 0 | 100 |
| TESTI2028488 | 0 | 0 | 0 | 0 | 0 | 0 | 100 |
| TESTI2028523 | 0 | 0 | 0 | 0 | 0 | 0 | 100 |
| TESTI2028583 | 0 | 0 | 0 | 0 | 0 | 0 | 100 |
| TESTI2028613 | 0 | 0 | 0 | 0 | 0 | 0 | 100 |
| TESTI2028659 | 0 | 0 | 0 | 0 | 0 | 0 | 19.704 |
| TESTI2028662 | 0 | 0 | 0 | 0 | 0 | 0 | 100 |
| TESTI2028670 | 0 | 0 | 0 | 0 | 0 | 0 | 100 |
| TESTI2028705 | 0 | 0 | 0 | 0 | 0 | 0 | 100 |
| TESTI2028776 | 0 | 0 | 0 | 0 | 0 | 0 | 100 |
| TESTI2028811 | 0 | 0 | 0 | 0 | 0 | 0 | 100 |
| TESTI2028891 | 0 | 0 | 0 | 0 | 0 | 0 | 100 |
| TESTI2028985 | 0 | 0 | 0 | 0 | 0 | 0 | 100 |
| TESTI2028997 | 0 | 0 | 0 | 0 | 0 | 0 | 100 |
| TESTI2029131 | 0 | 0 | 0 | 0 | 0 | 0 | 100 |
| TESTI2029162 | 0 | 0 | 0 | 0 | 0 | 0 | 100 |
| TESTI2029196 | 0 | 0 | 0 | 0 | 0 | 0 | 100 |
| TESTI2029201 | 0 | 0 | 0 | 0 | 0 | 0 | 100 |
| TESTI2029227 | 0 | 0 | 0 | 0 | 0 | 0 | 11.641 |
| TESTI2029244 | 0 | 0 | 0 | 0 | 0 | 0 | 100 |
| TESTI2029249 | 0 | 0 | 0 | 0 | 0 | 0 | 100 |
| TESTI2029252 | 0 | 0 | 0 | 0 | 0 | 0 | 100 |
| TESTI2029259 | 0 | 0 | 0 | 0 | 0 | 0 | 2.163 |
| TESTI2029264 | 0 | 0 | 0 | 0 | 0 | 0 | 100 |
| TESTI2029470 | 0 | 0 | 0 | 0 | 0 | 0 | 100 |
| TESTI2029672 | 0 | 0 | 0 | 0 | 0 | 0 | 100 |
| TESTI2029785 | 0 | 0 | 0 | 0 | 0 | 0 | 43.944 |
| TESTI2029880 | 0 | 0 | 0 | 0 | 0 | 0 | 100 |
| TESTI2030136 | 0 | 0 | 0 | 0 | 0 | 0 | 100 |
| TESTI2030322 | 0 | 0 | 0 | 0 | 0 | 0 | 100 |
| TESTI2030336 | 0 | 0 | 0 | 0 | 0 | 0 | 43.944 |
| TESTI2030342 | 0 | 0 | 0 | 0 | 0 | 0 | 100 |
| TESTI2030519 | 0 | 0 | 0 | 0 | 0 | 0 | 100 |
| TESTI2030554 | 0 | 0 | 0 | 0 | 0 | 0 | 70.165 |
| TESTI2030556 | 0 | 0 | 0 | 0 | 0 | 0 | 100 |
| TESTI2030728 | 0 | 0 | 0 | 0 | 0 | 0 | 100 |
| TESTI2030754 | 0 | 0 | 0 | 0 | 0 | 0 | 100 |
| TESTI2030770 | 0 | 0 | 0 | 0 | 0 | 0 | 100 |
| TESTI2030860 | 0 | 0 | 0 | 0 | 0 | 0 | 100 |
| TESTI2030901 | 0 | 0 | 0 | 0 | 0 | 0 | 100 |
| TESTI2030917 | 0 | 0 | 0 | 0 | 0 | 0 | 100 |
| TESTI2030930 | 0 | 0 | 0 | 0 | 0 | 0 | 100 |
| TESTI2031006 | 0 | 0 | 0 | 0 | 0 | 0 | 100 |
| TESTI2031007 | 0 | 0 | 0 | 0 | 0 | 0 | 63.646 |
| TESTI2031060 | 0 | 0 | 0 | 0 | 0 | 0 | 100 |
| TESTI2031129 | 0 | 0 | 0 | 0 | 0 | 0 | 100 |
| TESTI2031356 | 0 | 0 | 0 | 0 | 0 | 0 | 100 |
| TESTI2031418 | 0 | 0 | 0 | 0 | 0 | 0 | 100 |
| TESTI2031504 | 0 | 0 | 0 | 0 | 0 | 0 | 100 |
| TESTI2031529 | 0 | 0 | 0 | 0 | 0 | 0 | 100 |
| TESTI2031687 | 0 | 0 | 0 | 0 | 0 | 0 | 100 |
| TESTI2031760 | 0 | 0 | 0 | 0 | 0 | 0 | 100 |
| TESTI2031809 | 0 | 0 | 0 | 0 | 0 | 0 | 100 |
| TESTI2031919 | 0 | 0 | 0 | 0 | 0 | 0 | 100 |
| TESTI2031935 | 0 | 0 | 0 | 0 | 0 | 0 | 100 |
| TESTI2031986 | 0 | 0 | 0 | 0 | 0 | 0 | 100 |
| TESTI2032044 | 0 | 0 | 0 | 0 | 0 | 0 | 100 |
| TESTI2032067 | 0 | 0 | 0 | 0 | 0 | 0 | 100 |
| TESTI2032079 | 0 | 0 | 0 | 0 | 0 | 0 | 100 |
| TESTI2032643 | 0 | 0 | 0 | 0 | 0 | 0 | 7.949 |
| TESTI2032681 | 0 | 0 | 0 | 0 | 0 | 0 | 100 |
| TESTI2032761 | 0 | 0 | 0 | 0 | 0 | 0 | 100 |
| TESTI2032768 | 0 | 0 | 0 | 0 | 0 | 0 | 100 |
| TESTI2032774 | 0 | 0 | 0 | 0 | 0 | 0 | 100 |
| TESTI2032828 | 0 | 0 | 0 | 0 | 0 | 0 | 100 |
| TESTI2033031 | 0 | 0 | 0 | 0 | 0 | 0 | 40.225 |
| TESTI2033242 | 0 | 0 | 0 | 0 | 0 | 0 | 100 |
| TESTI2033254 | 0 | 0 | 0 | 0 | 0 | 0 | 100 |
| TESTI2033300 | 0 | 0 | 0 | 0 | 0 | 0 | 100 |
| TESTI2033395 | 0 | 0 | 0 | 0 | 0 | 0 | 100 |
| TESTI2033441 | 0 | 0 | 15.439 | 0 | 0 | 7.508 | 1.435 |
| TESTI2033453 | 0 | 0 | 0 | 0 | 0 | 0 | 100 |
| TESTI2033505 | 0 | 0 | 0 | 0 | 0 | 0 | 100 |
| TESTI2033520 | 0 | 0 | 0 | 0 | 0 | 0 | 100 |
| TESTI2033641 | 0 | 0 | 0 | 0 | 0 | 0 | 100 |
| TESTI2033710 | 0 | 0 | 0 | 0 | 0 | 0 | 100 |
| TESTI2033905 | 0 | 0 | 0 | 0 | 0 | 0 | 100 |
| TESTI2034187 | 0 | 0 | 0 | 0 | 0 | 0 | 100 |
| TESTI2034243 | 0 | 0 | 0 | 0 | 0 | 0 | 100 |
| TESTI2034249 | 0 | 0 | 0 | 0 | 0 | 0 | 79.187 |
| TESTI2034307 | 0 | 0 | 0 | 0 | 0 | 0 | 43.944 |
| TESTI2034357 | 0 | 0 | 0 | 0 | 0 | 0 | 100 |
| TESTI2034401 | 0 | 0 | 0 | 0 | 0 | 0 | 100 |
| TESTI2034407 | 0 | 0 | 0 | 0 | 0 | 0 | 100 |
| TESTI2034506 | 0 | 0 | 0 | 0 | 0 | 0 | 100 |
| TESTI2034520 | 0 | 0 | 0 | 0 | 0 | 0 | 100 |
| TESTI2034718 | 0 | 0 | 0 | 0 | 0 | 0 | 100 |
| TESTI2034730 | 0 | 0 | 0 | 0 | 0 | 0 | 100 |
| TESTI2034749 | 0 | 0 | 0 | 0 | 0 | 0 | 100 |
| TESTI2034767 | 0 | 0 | 0 | 0 | 0 | 0 | 100 |
| TESTI2034774 | 0 | 0 | 0 | 0 | 0 | 0 | 100 |
| TESTI2034777 | 0 | 0 | 0 | 0 | 0 | 0 | 100 |
| TESTI2034785 | 0 | 0 | 0 | 0 | 0 | 0 | 100 |
| TESTI2034799 | 0 | 0 | 0 | 0 | 0 | 0 | 100 |
| TESTI2034847 | 0 | 0 | 0 | 0 | 0 | 0 | 100 |
| TESTI2034913 | 0 | 0 | 0 | 0 | 0 | 0 | 100 |
| TESTI2034916 | 0 | 0 | 0 | 0 | 0 | 0 | 9.938 |
| TESTI2034931 | 0 | 0 | 0 | 0 | 0 | 0 | 100 |
| TESTI2034940 | 0 | 0 | 0 | 0 | 0 | 0 | 0.851 |
| TESTI2034953 | 0 | 0 | 0 | 0 | 0 | 0 | 100 |
| TESTI2034997 | 0 | 0 | 0 | 0 | 0 | 0 | 100 |
| TESTI2035003 | 0 | 0 | 0 | 0 | 0 | 0 | 47.943 |
| TESTI2035078 | 0 | 0 | 0 | 0 | 0 | 0 | 100 |
| TESTI2035084 | 0 | 0 | 0 | 0 | 0 | 0 | 100 |
| TESTI2035107 | 0 | 0 | 0 | 0 | 0 | 0 | 100 |
| TESTI2035121 | 0 | 0 | 0 | 0 | 0 | 0 | 100 |
| TESTI2035124 | 0 | 0 | 0 | 0 | 0 | 0 | 100 |
| TESTI2035183 | 0 | 0 | 0 | 0 | 0 | 0 | 100 |
| TESTI2035236 | 0 | 0 | 0 | 0 | 0 | 0 | 16.851 |
| TESTI2035262 | 0 | 0 | 0 | 0 | 0 | 0 | 100 |
| TESTI2035309 | 0 | 0 | 0 | 0 | 0 | 0 | 26.610 |
| TESTI2035688 | 0 | 0 | 0 | 0 | 0 | 0 | 100 |
| TESTI2035775 | 0 | 0 | 0 | 0 | 0 | 0 | 100 |
| TESTI2035793 | 0 | 0 | 0 | 0 | 0 | 0 | 100 |
| TESTI2035898 | 0 | 0 | 0 | 0 | 0 | 0 | 100 |
| TESTI2035962 | 0 | 0 | 0 | 0 | 0 | 0 | 100 |
| TESTI2035981 | 0 | 0 | 0 | 0 | 0 | 0 | 100 |
| TESTI2035997 | 0 | 0 | 0 | 0 | 0 | 0 | 100 |
| TESTI2036114 | 0 | 0 | 0 | 0 | 0 | 0 | 100 |
| TESTI2036129 | 0 | 0 | 0 | 0 | 0 | 0 | 100 |
| TESTI2036285 | 0 | 0 | 0 | 0 | 0 | 0 | 100 |
| TESTI2036288 | 0 | 0 | 0 | 0 | 0 | 0 | 100 |
| TESTI2036513 | 0 | 0 | 0 | 0 | 0 | 0 | 100 |
| TESTI2036589 | 0 | 0 | 0 | 0 | 0 | 0 | 100 |
| TESTI2036650 | 0 | 0 | 0 | 0 | 0 | 0 | 39.348 |
| TESTI2036684 | 0 | 0 | 0 | 0 | 0 | 0 | 100 |
| TESTI2036691 | 0 | 0 | 0 | 0 | 0 | 0 | 15.347 |
| TESTI2036736 | 0 | 0 | 0 | 0 | 0 | 0 | 100 |
| TESTI2036822 | 0 | 0 | 0 | 0 | 0 | 0 | 100 |
| TESTI2036833 | 0 | 0 | 0 | 0 | 0 | 0 | 100 |
| TESTI2036913 | 0 | 0 | 0 | 0 | 0 | 0 | 53.064 |
| TESTI2036922 | 0 | 0 | 0 | 0 | 0 | 0 | 100 |
| TESTI2036951 | 0 | 0 | 0 | 0 | 0 | 0 | 100 |
| TESTI2036965 | 0 | 0 | 0 | 0 | 0 | 0 | 100 |
| TESTI2036969 | 0 | 0 | 0 | 0 | 0 | 0 | 18.789 |
| TESTI2037002 | 0 | 0 | 0 | 0 | 0 | 0 | 100 |
| TESTI2037055 | 0 | 0 | 0 | 0 | 0 | 0 | 100 |
| TESTI2037081 | 0 | 0 | 0 | 0 | 0 | 0 | 100 |
| TESTI2037085 | 0 | 0 | 0 | 0 | 0 | 0 | 100 |
| TESTI2037103 | 0 | 0 | 0 | 0 | 0 | 0 | 100 |
| TESTI2037106 | 0 | 0 | 0 | 0 | 0 | 0 | 100 |
| TESTI2037111 | 0 | 0 | 0 | 0 | 0 | 0 | 100 |
| TESTI2037209 | 0 | 0 | 0 | 0 | 0 | 0 | 100 |
| TESTI2037255 | 0 | 0 | 0 | 0 | 0 | 0 | 100 |
| TESTI2037359 | 0 | 0 | 0 | 0 | 0 | 0 | 100 |
| TESTI2037375 | 0 | 0 | 0 | 0 | 0 | 0 | 100 |
| TESTI2037382 | 0 | 0 | 0 | 0 | 0 | 0 | 100 |
| TESTI2037534 | 0 | 0 | 0 | 0 | 0 | 0 | 100 |
| TESTI2037569 | 0 | 0 | 0 | 0 | 0 | 0 | 100 |
| TESTI2037572 | 0 | 0 | 0 | 0 | 0 | 0 | 100 |
| TESTI2037643 | 0 | 0 | 0 | 0 | 0 | 0 | 100 |
| TESTI2037657 | 0 | 0 | 0 | 0 | 0 | 0 | 100 |
| TESTI2037673 | 0 | 0 | 0 | 0 | 0 | 0 | 100 |
| TESTI2037723 | 0 | 0 | 0 | 0 | 0 | 0 | 100 |
| TESTI2037819 | 0 | 0 | 0 | 0 | 0 | 0 | 100 |
| TESTI2037830 | 0 | 0 | 0 | 0 | 0 | 0 | 100 |
| TESTI2037845 | 0 | 0 | 0 | 0 | 0 | 0 | 100 |
| TESTI2037877 | 0 | 0 | 0 | 0 | 0 | 0 | 100 |
| TESTI2037976 | 0 | 0 | 0 | 0 | 0 | 0 | 100 |
| TESTI2038048 | 0 | 0 | 0 | 0 | 0 | 0 | 100 |
| TESTI2038065 | 0 | 0 | 0 | 0 | 0 | 0 | 100 |
| TESTI2038104 | 0 | 0 | 0 | 0 | 0 | 0 | 100 |
| TESTI2038275 | 0 | 0 | 0 | 0 | 0 | 0 | 100 |
| TESTI2038388 | 0 | 0 | 0 | 0 | 0 | 0 | 100 |
| TESTI2038573 | 0 | 0 | 0 | 0 | 0 | 0 | 100 |
| TESTI2038596 | 0 | 0 | 0 | 0 | 0 | 0 | 100 |
| TESTI2038623 | 0 | 0 | 0 | 0 | 0 | 0 | 100 |
| TESTI2038644 | 0 | 0 | 0 | 0 | 0 | 0 | 100 |
| TESTI2038733 | 0 | 0 | 0 | 0 | 0 | 0 | 100 |
| TESTI2038858 | 0 | 0 | 0 | 0 | 0 | 0 | 100 |
| TESTI2038958 | 0 | 0 | 0 | 0 | 0 | 0 | 100 |
| TESTI2039025 | 0 | 0 | 0 | 0 | 0 | 0 | 100 |
| TESTI2039026 | 0 | 0 | 0 | 0 | 0 | 0 | 100 |
| TESTI2039038 | 0 | 0 | 0 | 0 | 0 | 0 | 100 |
| TESTI2039041 | 0 | 0 | 0 | 0 | 0 | 0 | 100 |
| TESTI2039060 | 0 | 0 | 0 | 0 | 0 | 0 | 100 |
| TESTI2039113 | 0 | 0 | 0 | 0 | 0 | 0 | 100 |
| TESTI2039121 | 0 | 0 | 0 | 0 | 0 | 0 | 100 |
| TESTI2039177 | 0 | 0 | 0 | 0 | 0 | 0 | 16.255 |
| TESTI2039209 | 0 | 0 | 0 | 0 | 0 | 0 | 100 |
| TESTI2039225 | 0 | 0 | 0 | 0 | 0 | 0 | 100 |
| TESTI2039227 | 0 | 0 | 0 | 0 | 0 | 0 | 100 |
| TESTI2039276 | 0 | 0 | 0 | 0 | 0 | 0 | 100 |
| TESTI2039342 | 0 | 0 | 0 | 0 | 0 | 0 | 22.764 |
| TESTI2039353 | 0 | 0 | 0 | 0 | 0 | 0 | 100 |
| TESTI2039544 | 0 | 0 | 0 | 0 | 0 | 0 | 100 |
| TESTI2039613 | 0 | 0 | 0 | 0 | 0 | 0 | 100 |
| TESTI2039707 | 0 | 0 | 0 | 0 | 0 | 0 | 15.009 |
| TESTI2039732 | 0 | 0 | 0 | 0 | 0 | 0 | 100 |
| TESTI2039738 | 0 | 0 | 0 | 0 | 0 | 0 | 100 |
| TESTI2039776 | 0 | 0 | 0 | 0 | 0 | 0 | 100 |
| TESTI2039867 | 0 | 0 | 0 | 0 | 0 | 0 | 100 |
| TESTI2039908 | 0 | 0 | 0 | 0 | 0 | 0 | 100 |
| TESTI2040018 | 0 | 0 | 0 | 0 | 0 | 0 | 100 |
| TESTI2040094 | 0 | 0 | 0 | 0 | 0 | 0 | 8.042 |
| TESTI2040102 | 0 | 0 | 0 | 0 | 0 | 0 | 100 |
| TESTI2040128 | 0 | 0 | 0 | 0 | 0 | 0 | 100 |
| TESTI2040143 | 0 | 0 | 0 | 0 | 0 | 0 | 100 |
| TESTI2040297 | 0 | 0 | 0 | 0 | 0 | 0 | 100 |
| TESTI2040372 | 0 | 0 | 0 | 0 | 0 | 0 | 100 |
| TESTI2040377 | 0 | 0 | 0 | 0 | 0 | 0 | 100 |
| TESTI2040424 | 0 | 0 | 0 | 0 | 0 | 0 | 100 |
| TESTI2040642 | 0 | 0 | 0 | 0 | 0 | 0 | 100 |
| TESTI2040815 | 0 | 0 | 0 | 0 | 0 | 0 | 100 |
| TESTI2040897 | 0 | 0 | 0 | 0 | 0 | 0 | 100 |
| TESTI2040944 | 0 | 0 | 0 | 0 | 0 | 0 | 100 |
| TESTI2040964 | 0 | 0 | 0 | 0 | 0 | 0 | 100 |
| TESTI2040983 | 0 | 0 | 0 | 0 | 0 | 0 | 100 |
| TESTI2040989 | 0 | 0 | 0 | 0 | 0 | 0 | 100 |
| TESTI2041330 | 0 | 0 | 0 | 0 | 0 | 0 | 100 |
| TESTI2041362 | 0 | 0 | 0 | 0 | 0 | 0 | 100 |
| TESTI2041517 | 0 | 0 | 0 | 0 | 0 | 0 | 100 |
| TESTI2041564 | 0 | 0 | 0 | 0 | 0 | 0 | 100 |
| TESTI2041664 | 0 | 0 | 0 | 0 | 0 | 0 | 100 |
| TESTI2041730 | 0 | 0 | 0 | 0 | 0 | 0 | 100 |
| TESTI2041734 | 0 | 0 | 0 | 0 | 0 | 0 | 51.276 |
| TESTI2041778 | 0 | 0 | 0 | 0 | 0 | 0 | 100 |
| TESTI2041800 | 0 | 0 | 0 | 0 | 0 | 0 | 100 |
| TESTI2041947 | 0 | 0 | 0 | 0 | 0 | 0 | 100 |
| TESTI2041956 | 0 | 0 | 0 | 0 | 0 | 0 | 100 |
| TESTI2041976 | 0 | 0 | 0 | 0 | 0 | 0 | 100 |
| TESTI2042062 | 0 | 0 | 0 | 0 | 0 | 0 | 100 |
| TESTI2042149 | 0 | 0 | 0 | 0 | 0 | 0 | 100 |
| TESTI2042264 | 0 | 0 | 0 | 0 | 0 | 0 | 100 |
| TESTI2042295 | 0 | 0 | 0 | 0 | 0 | 0 | 100 |
| TESTI2042302 | 0 | 0 | 0 | 0 | 0 | 0 | 100 |
| TESTI2042326 | 0 | 0 | 0 | 0 | 0 | 0 | 100 |
| TESTI2042400 | 0 | 0 | 0 | 0 | 0 | 0 | 100 |
| TESTI2042450 | 0 | 0 | 0 | 0 | 0 | 0 | 100 |
| TESTI2042473 | 0 | 0 | 0 | 0 | 0 | 0 | 100 |
| TESTI2042507 | 0 | 0 | 0 | 0 | 0 | 0 | 100 |
| TESTI2042508 | 0 | 0 | 0 | 0 | 0 | 0 | 100 |
| TESTI2042750 | 0 | 0 | 0 | 0 | 0 | 0 | 100 |
| TESTI2042783 | 0 | 0 | 0 | 0 | 0 | 0 | 100 |
| TESTI2042806 | 0 | 0 | 0 | 0 | 0 | 0 | 100 |
| TESTI2042928 | 0 | 0 | 0 | 0 | 0 | 0 | 100 |
| TESTI2042946 | 0 | 0 | 0 | 0 | 0 | 0 | 100 |
| TESTI2042958 | 0 | 0 | 0 | 0 | 0 | 0 | 100 |
| TESTI2043275 | 0 | 0 | 62.244 | 0 | 0 | 0 | 5.784 |
| TESTI2043277 | 0 | 0 | 0 | 0 | 0 | 0 | 100 |
| TESTI2043282 | 0 | 0 | 0 | 0 | 0 | 0 | 100 |
| TESTI2043313 | 0 | 0 | 0 | 0 | 0 | 0 | 100 |
| TESTI2043477 | 0 | 0 | 0 | 0 | 0 | 0 | 100 |
| TESTI2043585 | 0 | 0 | 0 | 0 | 0 | 0 | 100 |
| TESTI2043758 | 0 | 0 | 0 | 0 | 0 | 0 | 100 |
| TESTI2043817 | 0 | 0 | 0 | 0 | 0 | 0 | 100 |
| TESTI2043857 | 0 | 0 | 0 | 0 | 0 | 0 | 100 |
| TESTI2043866 | 0 | 0 | 0 | 0 | 0 | 0 | 100 |
| TESTI2043869 | 0 | 0 | 0 | 0 | 0 | 0 | 100 |
| TESTI2044064 | 0 | 0 | 0 | 0 | 0 | 0 | 100 |
| TESTI2044118 | 0 | 0 | 0 | 0 | 0 | 0 | 100 |
| TESTI2044194 | 0 | 0 | 0 | 0 | 0 | 0 | 100 |
| TESTI2044276 | 0 | 0 | 0 | 0 | 0 | 0 | 9.299 |
| TESTI2044306 | 0 | 0 | 0 | 0 | 0 | 0 | 100 |
| TESTI2044309 | 0 | 0 | 0 | 0 | 0 | 0 | 100 |
| TESTI2044413 | 0 | 0 | 0 | 0 | 0 | 0 | 100 |
| TESTI2044418 | 0 | 0 | 0 | 0 | 0 | 0 | 100 |
| TESTI2044754 | 0 | 0 | 0 | 0 | 0 | 0 | 100 |
| TESTI2044788 | 0 | 0 | 0 | 0 | 0 | 0 | 35.404 |
| TESTI2044796 | 0 | 0 | 0 | 0 | 0 | 0 | 100 |
| TESTI2044833 | 0 | 0 | 0 | 0 | 0 | 0 | 100 |
| TESTI2044892 | 0 | 0 | 0 | 0 | 0 | 0 | 100 |
| TESTI2044920 | 0 | 0 | 0 | 0 | 0 | 0 | 100 |
| TESTI2044968 | 0 | 0 | 0 | 0 | 0 | 0 | 100 |
| TESTI2045139 | 0 | 0 | 0 | 0 | 0 | 0 | 100 |
| TESTI2045155 | 0 | 0 | 0 | 0 | 0 | 0 | 7.529 |
| TESTI2045171 | 0 | 0 | 0 | 0 | 0 | 0 | 100 |
| TESTI2045199 | 0 | 0 | 0 | 0 | 0 | 0 | 100 |
| TESTI2045353 | 0 | 0 | 0 | 0 | 0 | 0 | 100 |
| TESTI2045509 | 0 | 0 | 0 | 0 | 0 | 0 | 100 |
| TESTI2045562 | 0 | 0 | 0 | 0 | 0 | 0 | 15.347 |
| TESTI2045611 | 0 | 0 | 0 | 0 | 0 | 0 | 100 |
| TESTI2045819 | 0 | 0 | 0 | 0 | 0 | 0 | 100 |
| TESTI2045850 | 0 | 0 | 0 | 0 | 0 | 0 | 100 |
| TESTI2045920 | 0 | 0 | 0 | 0 | 0 | 0 | 100 |
| TESTI2045964 | 0 | 0 | 0 | 0 | 0 | 0 | 100 |
| TESTI2045983 | 0 | 0 | 0 | 0 | 0 | 0 | 100 |
| TESTI2046100 | 0 | 0 | 0 | 0 | 0 | 0 | 100 |
| TESTI2046188 | 0 | 0 | 0 | 0 | 0 | 0 | 100 |
| TESTI2046347 | 0 | 0 | 0 | 0 | 0 | 0 | 100 |
| TESTI2046352 | 0 | 0 | 0 | 0 | 0 | 0 | 100 |
| TESTI2046358 | 0 | 0 | 0 | 0 | 0 | 0 | 100 |
| TESTI2046439 | 0 | 0 | 0 | 0 | 0 | 0 | 100 |
| TESTI2046456 | 0.998 | 0 | 0 | 0 | 0 | 3.167 | 5.446 |
| TESTI2046535 | 0 | 0 | 0 | 0 | 0 | 0 | 100 |
| TESTI2046536 | 0 | 0 | 0 | 0 | 0 | 0 | 100 |
| TESTI2046552 | 0 | 0 | 0 | 0 | 0 | 0 | 100 |
| TESTI2046569 | 0 | 0 | 0 | 0 | 0 | 0 | 100 |
| TESTI2046686 | 0 | 0 | 0 | 0 | 0 | 0 | 100 |
| TESTI2046721 | 0 | 0 | 0 | 0 | 0 | 0 | 8.001 |
| TESTI2046732 | 0 | 0 | 0 | 0 | 0 | 0 | 100 |
| TESTI2046786 | 0 | 0 | 0 | 0 | 0 | 0 | 100 |
| TESTI2046797 | 0 | 0 | 0 | 0 | 0 | 0 | 100 |
| TESTI2046863 | 0 | 0 | 0 | 0 | 0 | 0 | 100 |
| TESTI2046872 | 0 | 0 | 0 | 0 | 0 | 0 | 100 |
| TESTI2047071 | 0 | 0 | 0 | 0 | 0 | 0 | 100 |
| TESTI2047141 | 0 | 0 | 0 | 0 | 0 | 0 | 100 |
| TESTI2047147 | 0 | 0 | 0 | 0 | 0 | 0 | 100 |
| TESTI2047153 | 0 | 0 | 0 | 0 | 0 | 0 | 100 |
| TESTI2047212 | 0 | 0 | 0 | 0 | 0 | 0 | 100 |
| TESTI2047342 | 0 | 0 | 0 | 0 | 0 | 0 | 100 |
| TESTI2047383 | 0 | 0 | 0 | 0 | 0 | 0 | 100 |
| TESTI2047605 | 31.427 | 0 | 0 | 0 | 0 | 0 | 9.525 |
| TESTI2047792 | 0 | 0 | 0 | 0 | 0 | 0 | 100 |
| TESTI2047801 | 0 | 0 | 0 | 0 | 0 | 0 | 100 |
| TESTI2047818 | 0 | 0 | 0 | 0 | 0 | 0 | 100 |
| TESTI2047824 | 0 | 0 | 0 | 0 | 0 | 0 | 100 |
| TESTI2047885 | 0 | 0 | 0 | 0 | 0 | 0 | 100 |
| TESTI2047930 | 0 | 0 | 0 | 0 | 0 | 0 | 100 |
| TESTI2048105 | 0 | 0 | 0 | 0 | 0 | 0 | 100 |
| TESTI2048109 | 0 | 0 | 0 | 0 | 0 | 0 | 100 |
| TESTI2048465 | 0 | 0 | 0 | 0 | 0 | 0 | 100 |
| TESTI2048603 | 0 | 0 | 0 | 0 | 0 | 0 | 100 |
| TESTI2048898 | 0 | 0 | 0 | 0 | 0 | 0 | 100 |
| TESTI2049041 | 0 | 0 | 0 | 0 | 0 | 0 | 100 |
| TESTI2049062 | 0 | 0 | 0 | 0 | 0 | 0 | 100 |
| TESTI2049206 | 0 | 0 | 0 | 0 | 0 | 0 | 100 |
| TESTI2049246 | 0 | 0 | 0 | 0 | 0 | 0 | 17.188 |
| TESTI2049277 | 0 | 0 | 0 | 0 | 0 | 0 | 100 |
| TESTI2049422 | 0 | 0 | 0 | 0 | 0 | 0 | 100 |
| TESTI2049452 | 0 | 0 | 0 | 0 | 0 | 0 | 100 |
| TESTI2049469 | 0 | 0 | 0 | 0 | 0 | 0 | 100 |
| TESTI2049553 | 0 | 0 | 0 | 0 | 0 | 0 | 100 |
| TESTI2049576 | 0 | 0 | 0 | 0 | 0 | 0 | 100 |
| TESTI2049857 | 0 | 0 | 0 | 0 | 0 | 0 | 100 |
| TESTI2049956 | 0 | 0 | 0 | 0 | 0 | 0 | 100 |
| TESTI2050137 | 0 | 0 | 0 | 0 | 0 | 0 | 100 |
| TESTI2050681 | 0 | 0 | 0 | 0 | 0 | 0 | 100 |
| TESTI2050780 | 0 | 0 | 0 | 0 | 0 | 0 | 100 |
| TESTI2050987 | 0 | 0 | 0 | 0 | 0 | 0 | 100 |
| TESTI2051148 | 0 | 0 | 0 | 0 | 0 | 0 | 100 |
| TESTI2051177 | 0 | 0 | 0 | 0 | 0 | 0 | 15.630 |
| TESTI2051279 | 0 | 0 | 0 | 0 | 0 | 0 | 100 |
| TESTI2051488 | 0 | 0 | 0 | 0 | 0 | 0 | 100 |
| TESTI2051543 | 0 | 0 | 0 | 0 | 0 | 0 | 100 |
| TESTI2051627 | 0 | 0 | 0 | 0 | 0 | 0 | 100 |
| TESTI2051742 | 0 | 0 | 0 | 0 | 0 | 0 | 100 |
| TESTI2051767 | 0 | 0 | 0 | 0 | 0 | 0 | 100 |
| TESTI2051791 | 0 | 0 | 0 | 0 | 0 | 0 | 100 |
| TESTI2051806 | 0 | 0 | 0 | 0 | 0 | 0 | 100 |
| TESTI2051867 | 0 | 0 | 0 | 0 | 0 | 0 | 100 |
| TESTI2051873 | 0 | 0 | 0 | 0 | 0 | 0 | 100 |
| TESTI2052110 | 0 | 0 | 0 | 0 | 0 | 0 | 100 |
| TESTI2052202 | 0 | 0 | 0 | 0 | 0 | 0 | 100 |
| TESTI2052211 | 0 | 0 | 0 | 0 | 0 | 0 | 100 |
| TESTI2052256 | 0 | 0 | 0 | 0 | 0 | 0 | 100 |
| TESTI2052288 | 0 | 0 | 0 | 0 | 0 | 0 | 100 |
| TESTI2052525 | 0 | 0 | 0 | 0 | 0 | 0 | 100 |
| TESTI2052670 | 0 | 0 | 0 | 0 | 0 | 0 | 100 |
| TESTI2052693 | 0 | 0 | 0 | 0 | 35.495 | 0 | 1.094 |
| TESTI2052698 | 0 | 0 | 0 | 0 | 0 | 0 | 100 |
| TESTI2052799 | 0 | 0 | 0 | 0 | 0 | 0 | 100 |
| TESTI2052822 | 0 | 0 | 0 | 0 | 0 | 0 | 100 |
| TESTI2052985 | 0 | 0 | 0 | 0 | 0 | 0 | 100 |
| TESTI2053151 | 0 | 0 | 0 | 0 | 0 | 0 | 100 |
| TESTI2053242 | 0 | 0 | 0 | 0 | 0 | 0 | 100 |
| TESTI2053399 | 0 | 0 | 0 | 0 | 0 | 0 | 100 |
| TESTI2053526 | 0 | 0 | 0 | 0 | 0 | 0 | 100 |
| TESTI2053561 | 0 | 0 | 0 | 0 | 0 | 0 | 100 |
| TESTI2053621 | 0 | 0 | 0 | 0 | 0 | 0 | 100 |
| TESTI2053667 | 0 | 0 | 0 | 0 | 0 | 0 | 36.852 |
| TESTI2053723 | 0 | 0 | 0 | 0 | 0 | 0 | 2.959 |
| TESTI3000002 | 0 | 0 | 0 | 0 | 0 | 0 | 100 |
| TESTI3000005 | 0 | 0 | 0 | 0 | 0 | 0 | 100 |
| TESTI4000014 | 0 | 0 | 1.256 | 0 | 0 | 0 | 3.385 |
| TESTI4000068 | 0 | 0 | 0 | 0 | 0 | 0 | 100 |
| TESTI4000079 | 0 | 0 | 0 | 0 | 0 | 0 | 100 |
| TESTI4000093 | 0 | 0 | 0 | 0 | 0 | 0 | 100 |
| TESTI4000134 | 0 | 0 | 0 | 0 | 0 | 0 | 100 |
| TESTI4000137 | 0 | 0 | 0 | 0 | 0 | 0 | 100 |
| TESTI4000153 | 0 | 0 | 0 | 0 | 0 | 0 | 100 |
| TESTI4000155 | 0 | 0 | 0 | 0 | 0 | 0 | 100 |
| TESTI4000157 | 0 | 0 | 0 | 0 | 0 | 0 | 100 |
| TESTI4000183 | 0 | 0 | 0 | 0 | 0 | 0 | 100 |
| TESTI4000214 | 0 | 0 | 0 | 0 | 0 | 0 | 92.021 |
| TESTI4000215 | 0 | 0 | 0 | 0 | 0 | 0 | 100 |
| TESTI4000228 | 0 | 0 | 0 | 0 | 0 | 0 | 100 |
| TESTI4000250 | 0 | 0 | 0 | 0 | 0 | 0 | 100 |
| TESTI4000288 | 0 | 0 | 0 | 0 | 0 | 0 | 100 |
| TESTI4000319 | 0 | 0 | 0 | 0 | 0 | 0 | 100 |
| TESTI4000349 | 0 | 0 | 0 | 0 | 0 | 0 | 33.984 |
| TESTI4000370 | 0 | 0 | 0 | 0 | 0 | 0 | 100 |
| TESTI4000394 | 0 | 0 | 0 | 0 | 0 | 0 | 100 |
| TESTI4000434 | 0 | 0 | 0 | 0 | 0 | 0 | 59.830 |
| TESTI4000462 | 0 | 0 | 0 | 0 | 0 | 0 | 100 |
| TESTI4000530 | 0 | 0 | 0 | 0 | 0 | 0 | 100 |
| TESTI4000534 | 0 | 0 | 0 | 0 | 0 | 0 | 100 |
| TESTI4000598 | 0 | 0 | 0 | 0 | 0 | 0 | 100 |
| TESTI4000600 | 0 | 0 | 0 | 0 | 0 | 0 | 100 |
| TESTI4000621 | 0 | 0 | 0 | 0 | 0 | 0 | 100 |
| TESTI4000703 | 0 | 0 | 0 | 0 | 0 | 0 | 100 |
| TESTI4000724 | 0 | 0 | 0 | 0 | 0 | 0 | 100 |
| TESTI4000957 | 0 | 0 | 0 | 0 | 0 | 0 | 100 |
| TESTI4000970 | 0 | 0 | 0 | 0 | 0 | 0 | 100 |
| TESTI4001036 | 0 | 0 | 0 | 0 | 0 | 0 | 100 |
| TESTI4001037 | 0 | 0 | 0 | 0 | 0 | 0 | 100 |
| TESTI4001060 | 0 | 0 | 0 | 0 | 0 | 0 | 100 |
| TESTI4001106 | 0 | 0 | 0 | 0 | 0 | 0 | 100 |
| TESTI4001148 | 0 | 0 | 0 | 0 | 0 | 0 | 100 |
| TESTI4001176 | 0 | 0 | 0 | 0 | 0 | 0 | 100 |
| TESTI4001195 | 0 | 0 | 0 | 0 | 0 | 0 | 100 |
| TESTI4001201 | 0 | 0 | 0 | 0 | 0 | 0 | 100 |
| TESTI4001206 | 0 | 0 | 0 | 0 | 0 | 0 | 27.313 |
| TESTI4001348 | 0 | 0 | 0 | 0 | 0 | 0 | 100 |
| TESTI4001441 | 0 | 0 | 0 | 0 | 0 | 0 | 100 |
| TESTI4001467 | 0 | 0 | 0 | 0 | 0 | 0 | 3.619 |
| TESTI4001473 | 0 | 0 | 0 | 0 | 0 | 0 | 100 |
| TESTI4001517 | 0 | 0 | 0 | 0 | 0 | 0 | 100 |
| TESTI4001527 | 0 | 0 | 0 | 0 | 0 | 0 | 100 |
| TESTI4001561 | 0 | 0 | 0 | 0 | 0 | 0 | 26.320 |
| TESTI4001569 | 0 | 0 | 0 | 0 | 0 | 0 | 56.168 |
| TESTI4001604 | 0 | 0 | 0 | 0 | 0 | 0 | 38.043 |
| TESTI4001665 | 0 | 0 | 0 | 0 | 0 | 0 | 100 |
| TESTI4001679 | 0 | 0 | 0 | 0 | 0 | 0 | 100 |
| TESTI4001795 | 0 | 0 | 0 | 0 | 0 | 0 | 100 |
| TESTI4001923 | 0 | 0 | 0 | 0 | 0 | 0 | 100 |
| TESTI4001925 | 0 | 0 | 0 | 0 | 0 | 0 | 100 |
| TESTI4001984 | 0 | 0 | 0 | 0 | 0 | 0 | 39.529 |
| TESTI4002003 | 0 | 0 | 0 | 0 | 0 | 0 | 100 |
| TESTI4002072 | 0 | 0 | 0 | 0 | 0 | 0 | 2.183 |
| TESTI4002141 | 0 | 0 | 0 | 0 | 0 | 0 | 100 |
| TESTI4002195 | 0 | 0 | 0 | 0 | 0 | 0 | 8.747 |
| TESTI4002202 | 0 | 0 | 0 | 0 | 0 | 22.682 | 2.167 |
| TESTI4002290 | 0 | 0 | 0 | 0 | 0 | 0 | 37.085 |
| TESTI4002319 | 0 | 0 | 0 | 0 | 0 | 0 | 100 |
| TESTI4002491 | 0 | 0 | 0 | 0 | 0 | 0 | 100 |
| TESTI4002520 | 0 | 0 | 0 | 0 | 0 | 0 | 100 |
| TESTI4002552 | 0 | 0 | 0 | 0 | 0 | 0 | 100 |
| TESTI4002647 | 0 | 0 | 0 | 0 | 0 | 0 | 34.478 |
| TESTI4002703 | 0 | 0 | 0 | 0 | 0 | 0 | 100 |
| TESTI4002754 | 0 | 0 | 0 | 0 | 0 | 0 | 100 |
| TESTI4002799 | 0 | 0 | 0 | 0 | 0 | 0 | 2.887 |
| TESTI4002868 | 0 | 0 | 0 | 0 | 0 | 0 | 100 |
| TESTI4002878 | 0 | 0 | 0 | 0 | 0 | 0 | 100 |
| TESTI4002889 | 0 | 0 | 0 | 0 | 0 | 0 | 100 |
| TESTI4002988 | 0 | 0 | 0 | 0 | 0 | 0 | 14.176 |
| TESTI4003150 | 0 | 0 | 0 | 0 | 0 | 0 | 19.415 |
| TESTI4003179 | 0 | 0 | 0 | 0 | 0 | 0 | 53.856 |
| TESTI4003279 | 0 | 0 | 0 | 0 | 0 | 0 | 100 |
| TESTI4003319 | 0 | 0 | 0 | 0 | 0 | 0 | 100 |
| TESTI4003404 | 0 | 0 | 0 | 0 | 0 | 0 | 100 |
| TESTI4003565 | 0 | 0 | 0 | 0 | 0 | 0 | 100 |
| TESTI4003574 | 0 | 0 | 0 | 0 | 0 | 0 | 100 |
| TESTI4003579 | 0 | 0 | 0 | 0 | 0 | 0 | 36.852 |
| TESTI4003602 | 0 | 0 | 0 | 0 | 0 | 0 | 33.949 |
| TESTI4003703 | 0 | 0 | 0 | 0 | 0 | 0 | 23.140 |
| TESTI4003733 | 0 | 0 | 0 | 0 | 0 | 0 | 100 |
| TESTI4003796 | 0 | 0 | 0 | 0 | 0 | 0 | 100 |
| TESTI4003944 | 0 | 0 | 0 | 0 | 0 | 0 | 22.803 |
| TESTI4004003 | 0 | 0 | 0 | 0 | 0 | 0 | 100 |
| TESTI4004031 | 0 | 0 | 0 | 0 | 0 | 0 | 100 |
| TESTI4004200 | 0 | 0 | 0 | 0 | 0 | 0 | 100 |
| TESTI4004210 | 0 | 0 | 0 | 0 | 0 | 0 | 23.566 |
| TESTI4004539 | 0 | 0 | 0 | 0 | 0 | 0 | 100 |
| TESTI4004626 | 0 | 0 | 0 | 0 | 0 | 0 | 10.762 |
| TESTI4004653 | 0 | 0 | 0 | 0 | 0 | 0 | 100 |
| TESTI4004695 | 0 | 0 | 0 | 0 | 0 | 0 | 63.878 |
| TESTI4004722 | 0 | 0 | 0 | 0 | 0 | 0 | 100 |
| TESTI4004887 | 0 | 0 | 0 | 0 | 0 | 0 | 100 |
| TESTI4004917 | 0 | 0 | 0 | 0 | 0 | 0 | 100 |
| TESTI4005015 | 0 | 0 | 0 | 0 | 0 | 0 | 100 |
| TESTI4005039 | 0 | 0 | 0 | 0 | 0 | 0 | 100 |
| TESTI4005133 | 0 | 0 | 0 | 0 | 0 | 0 | 7.795 |
| TESTI4005158 | 0 | 0 | 0 | 0 | 0 | 0 | 100 |
| TESTI4005317 | 0 | 0 | 0 | 0 | 0 | 0 | 100 |
| TESTI4005322 | 0 | 0 | 0 | 0 | 0 | 0 | 100 |
| TESTI4005399 | 0 | 0 | 0 | 0 | 0 | 0 | 15.291 |
| TESTI4005470 | 0 | 0 | 0 | 0 | 0 | 0 | 100 |
| TESTI4005500 | 0 | 0 | 0 | 0 | 0 | 0 | 100 |
| TESTI4005534 | 0 | 0 | 0 | 0 | 0 | 0 | 18.229 |
| TESTI4005543 | 0 | 0 | 0 | 0 | 0 | 0 | 100 |
| TESTI4005628 | 0 | 0 | 0 | 0 | 0 | 0 | 100 |
| TESTI4005801 | 0 | 0 | 0 | 0 | 0 | 0 | 100 |
| TESTI4005805 | 0 | 0 | 0 | 0 | 0 | 0 | 100 |
| TESTI4005857 | 0 | 0 | 0 | 0 | 0 | 0 | 20.775 |
| TESTI4005961 | 0 | 0 | 0 | 0 | 0 | 0 | 100 |
| TESTI4006053 | 0 | 0 | 0 | 0 | 0 | 0 | 100 |
| TESTI4006079 | 0 | 0 | 0 | 0 | 0 | 0 | 100 |
| TESTI4006112 | 0 | 0 | 0 | 0 | 0 | 0 | 100 |
| TESTI4006137 | 0 | 0 | 0 | 0 | 0 | 0 | 16.835 |
| TESTI4006148 | 0 | 0 | 0 | 0 | 0 | 0 | 100 |
| TESTI4006219 | 0 | 0 | 0 | 0 | 0 | 0 | 100 |
| TESTI4006234 | 0 | 0 | 0 | 0 | 0 | 0 | 100 |
| TESTI4006308 | 0 | 0 | 0 | 0 | 0 | 0 | 100 |
| TESTI4006326 | 0 | 0 | 0 | 0 | 0 | 0 | 51.276 |
| TESTI4006393 | 0 | 0 | 0 | 0 | 0 | 0 | 100 |
| TESTI4006407 | 0 | 0 | 0 | 0 | 0 | 0 | 37.085 |
| TESTI4006412 | 0 | 0 | 0 | 0 | 0 | 0 | 100 |
| TESTI4006420 | 0 | 0 | 0 | 0 | 0 | 0 | 100 |
| TESTI4006441 | 0 | 0 | 0 | 0 | 0 | 0 | 39.529 |
| TESTI4006473 | 0 | 0 | 0 | 0 | 0 | 0 | 100 |
| TESTI4006539 | 0 | 0 | 0 | 0 | 0 | 0 | 100 |
| TESTI4006546 | 0 | 0 | 0 | 0 | 0 | 0 | 100 |
| TESTI4006567 | 0 | 0 | 0 | 0 | 0 | 0 | 100 |
| TESTI4006704 | 0 | 0 | 0 | 0 | 0 | 0 | 100 |
| TESTI4006728 | 0 | 0 | 0 | 0 | 0 | 0 | 100 |
| TESTI4006802 | 0 | 0 | 0 | 0 | 0 | 0 | 100 |
| TESTI4006819 | 0 | 0 | 0 | 0 | 0 | 0 | 53.856 |
| TESTI4007064 | 0 | 0 | 0 | 0 | 0 | 0 | 100 |
| TESTI4007163 | 0 | 0 | 0 | 0 | 0 | 0 | 100 |
| TESTI4007176 | 0 | 0 | 0 | 0 | 0 | 0 | 100 |
| TESTI4007203 | 0 | 0 | 0 | 0 | 0 | 0 | 100 |
| TESTI4007239 | 0 | 0 | 0 | 0 | 0 | 0 | 100 |
| TESTI4007369 | 0 | 0 | 0 | 0 | 0 | 0 | 8.520 |
| TESTI4007373 | 0 | 0 | 0 | 0 | 0 | 0 | 100 |
| TESTI4007382 | 0 | 0 | 0 | 0 | 0 | 0 | 100 |
| TESTI4007404 | 0 | 0 | 0 | 0 | 0 | 0 | 100 |
| TESTI4007489 | 0 | 0 | 0 | 0 | 0 | 0 | 100 |
| TESTI4007565 | 0 | 0 | 0 | 0 | 0 | 0 | 100 |
| TESTI4007671 | 0 | 0 | 0 | 0 | 0 | 0 | 100 |
| TESTI4007775 | 0 | 0 | 0 | 0 | 0 | 0 | 100 |
| TESTI4007778 | 0 | 0 | 0 | 0 | 0 | 0 | 100 |
| TESTI4007799 | 0 | 0 | 0 | 0 | 0 | 0 | 100 |
| TESTI4007810 | 0 | 0 | 0 | 0 | 0 | 0 | 100 |
| TESTI4007816 | 0 | 0 | 0 | 0 | 0 | 0 | 2.831 |
| TESTI4007965 | 0 | 0 | 0 | 0 | 0 | 0 | 47.189 |
| TESTI4008007 | 0 | 0 | 0 | 0 | 0 | 0 | 100 |
| TESTI4008018 | 0 | 0 | 0 | 0 | 0 | 0 | 25.979 |
| TESTI4008050 | 0 | 0 | 0 | 0 | 0 | 0 | 100 |
| TESTI4008058 | 0 | 0 | 0 | 0 | 0 | 0 | 17.285 |
| TESTI4008086 | 0 | 0 | 0 | 0 | 0 | 0 | 100 |
| TESTI4008097 | 0 | 0 | 0 | 0 | 0 | 0 | 39.529 |
| TESTI4008176 | 0 | 0 | 0 | 0 | 0 | 0 | 100 |
| TESTI4008209 | 0 | 0 | 0 | 0 | 0 | 0 | 100 |
| TESTI4008219 | 0 | 0 | 0 | 0 | 0 | 0 | 100 |
| TESTI4008250 | 0 | 0 | 0 | 0 | 0 | 0 | 100 |
| TESTI4008302 | 0 | 0 | 0 | 0 | 0 | 0 | 100 |
| TESTI4008305 | 0 | 0 | 0 | 0 | 0 | 0 | 100 |
| TESTI4008401 | 0 | 0 | 0 | 0 | 0 | 0 | 100 |
| TESTI4008417 | 0 | 0 | 0 | 0 | 0 | 0 | 100 |
| TESTI4008429 | 0 | 0 | 0 | 0 | 0 | 0 | 100 |
| TESTI4008573 | 0 | 0 | 0 | 0 | 0 | 0 | 100 |
| TESTI4008797 | 0 | 0 | 0 | 0 | 0 | 0 | 32.499 |
| TESTI4008816 | 0 | 0 | 0 | 0 | 0 | 0 | 100 |
| TESTI4008840 | 0 | 0 | 0 | 0 | 0 | 0 | 100 |
| TESTI4008935 | 0 | 0 | 0 | 0 | 0 | 0 | 100 |
| TESTI4008993 | 0 | 0 | 0 | 0 | 0 | 0 | 100 |
| TESTI4009022 | 0 | 0 | 0 | 0 | 0 | 0 | 100 |
| TESTI4009028 | 0 | 0 | 0 | 0 | 0 | 0 | 100 |
| TESTI4009034 | 0 | 0 | 0 | 0 | 0 | 0 | 100 |
| TESTI4009123 | 0 | 0 | 0 | 0 | 0 | 0 | 100 |
| TESTI4009160 | 0 | 0 | 0 | 0 | 0 | 0 | 100 |
| TESTI4009215 | 0 | 0 | 0 | 0 | 0 | 0 | 100 |
| TESTI4009283 | 0 | 0 | 0 | 0 | 0 | 0 | 100 |
| TESTI4009286 | 0 | 0 | 0 | 0 | 0 | 9.920 | 4.265 |
| TESTI4009306 | 0 | 0 | 0 | 0 | 0 | 0 | 100 |
| TESTI4009374 | 0 | 0 | 0 | 0 | 0 | 0 | 100 |
| TESTI4009406 | 0 | 0 | 0 | 0 | 0 | 0 | 100 |
| TESTI4009454 | 0 | 0 | 0 | 0 | 0 | 0 | 2.627 |
| TESTI4009457 | 0 | 0 | 0 | 0 | 0 | 0 | 70.009 |
| TESTI4009501 | 0 | 0 | 0 | 0 | 0 | 0 | 100 |
| TESTI4009563 | 0 | 0 | 0 | 0 | 0 | 0 | 100 |
| TESTI4009608 | 0 | 0 | 0 | 0 | 0 | 0 | 100 |
| TESTI4009638 | 0 | 0 | 0 | 0 | 0 | 0 | 100 |
| TESTI4009752 | 0 | 0 | 0 | 0 | 0 | 0 | 100 |
| TESTI4009881 | 0 | 0 | 0 | 0 | 0 | 0 | 100 |
| TESTI4009886 | 0 | 0 | 0 | 0 | 0 | 0 | 100 |
| TESTI4009928 | 0 | 0 | 0 | 0 | 0 | 0 | 100 |
| TESTI4010076 | 0 | 0 | 0 | 0 | 0 | 0 | 100 |
| TESTI4010082 | 0 | 0 | 0 | 0 | 0 | 0 | 100 |
| TESTI4010095 | 0 | 0 | 0 | 0 | 0 | 0 | 100 |
| TESTI4010211 | 0 | 0 | 0 | 0 | 0 | 0 | 100 |
| TESTI4010377 | 0 | 0 | 0 | 0 | 0 | 0 | 39.051 |
| TESTI4010475 | 0 | 0 | 0 | 0 | 0 | 0 | 100 |
| TESTI4010544 | 0 | 0 | 0 | 0 | 0 | 0 | 100 |
| TESTI4010713 | 0 | 0 | 0 | 0 | 0 | 0 | 100 |
| TESTI4010721 | 0 | 0 | 0 | 0 | 0 | 0 | 100 |
| TESTI4010789 | 0 | 0 | 0 | 0 | 0 | 0 | 100 |
| TESTI4010817 | 0 | 0 | 0 | 0 | 0 | 0 | 100 |
| TESTI4010831 | 0 | 0 | 0 | 0 | 0 | 0 | 100 |
| TESTI4010851 | 3.537 | 12.143 | 0 | 0 | 0 | 3.740 | 4.288 |
| TESTI4010902 | 0 | 0 | 0 | 0 | 0 | 0 | 100 |
| TESTI4010928 | 0 | 0 | 0 | 0 | 0 | 0 | 65.779 |
| TESTI4010979 | 0 | 0 | 0 | 0 | 0 | 0 | 12.844 |
| TESTI4011070 | 0 | 0 | 0 | 0 | 0 | 0 | 100 |
| TESTI4011072 | 0 | 0 | 0 | 0 | 0 | 0 | 100 |
| TESTI4011084 | 0 | 0 | 0 | 0 | 0 | 0 | 39.051 |
| TESTI4011118 | 0 | 0 | 0 | 0 | 0 | 0 | 100 |
| TESTI4011246 | 0 | 0 | 0 | 0 | 0 | 0 | 100 |
| TESTI4011484 | 0 | 0 | 0 | 0 | 0 | 0 | 100 |
| TESTI4011505 | 0 | 0 | 0 | 0 | 0 | 0 | 100 |
| TESTI4011532 | 0 | 0 | 0 | 0 | 0 | 0 | 100 |
| TESTI4011616 | 0 | 0 | 0 | 0 | 0 | 0 | 100 |
| TESTI4011744 | 0 | 0 | 0 | 0 | 0 | 0 | 100 |
| TESTI4011745 | 0 | 0 | 0 | 0 | 0 | 0 | 100 |
| TESTI4011829 | 0 | 0 | 0 | 0 | 0 | 0 | 100 |
| TESTI4011926 | 0 | 0 | 0 | 0 | 0 | 0 | 100 |
| TESTI4011956 | 0 | 0 | 0 | 0 | 0 | 0 | 100 |
| TESTI4012010 | 0 | 0 | 0 | 0 | 0 | 0 | 100 |
| TESTI4012086 | 0 | 0 | 0 | 0 | 0 | 0 | 100 |
| TESTI4012258 | 0 | 0 | 0 | 0 | 0 | 0 | 100 |
| TESTI4012293 | 0 | 0 | 0 | 0 | 0 | 0 | 100 |
| TESTI4012329 | 0 | 0 | 0 | 0 | 0 | 0 | 100 |
| TESTI4012382 | 0 | 0 | 0 | 0 | 0 | 0 | 100 |
| TESTI4012406 | 0 | 0 | 0 | 0 | 0 | 0 | 100 |
| TESTI4012448 | 0 | 0 | 0 | 0 | 0 | 0 | 100 |
| TESTI4012451 | 0 | 0 | 0 | 0 | 0 | 0 | 37.085 |
| TESTI4012505 | 0 | 0 | 0 | 0 | 0 | 0 | 100 |
| TESTI4012556 | 0 | 0 | 0 | 0 | 0 | 0 | 100 |
| TESTI4012623 | 0 | 0 | 0 | 0 | 0 | 0 | 100 |
| TESTI4012646 | 0 | 0 | 0 | 0 | 0 | 0 | 39.529 |
| TESTI4012679 | 0 | 0 | 0 | 0 | 0 | 0 | 100 |
| TESTI4012911 | 0 | 0 | 0 | 0 | 0 | 0 | 100 |
| TESTI4012956 | 0 | 0 | 0 | 0 | 0 | 0 | 100 |
| TESTI4012960 | 0 | 0 | 0 | 0 | 0 | 0 | 9.733 |
| TESTI4013365 | 0 | 0 | 0 | 0 | 0 | 0 | 100 |
| TESTI4013369 | 0 | 0 | 0 | 0 | 0 | 0 | 100 |
| TESTI4013410 | 0 | 0 | 0 | 0 | 0 | 0 | 100 |
| TESTI4013441 | 0.338 | 0 | 0 | 0 | 0 | 2.146 | 0.205 |
| TESTI4013474 | 2.229 | 0 | 0 | 0 | 0 | 0 | 0.675 |
| TESTI4013602 | 0 | 0 | 0 | 0 | 0 | 0 | 100 |
| TESTI4013667 | 0 | 0 | 0 | 0 | 0 | 0 | 100 |
| TESTI4013675 | 0 | 0 | 0 | 0 | 0 | 0 | 32.269 |
| TESTI4013685 | 0 | 0 | 0 | 0 | 0 | 0 | 100 |
| TESTI4013735 | 0 | 0 | 0 | 0 | 0 | 0 | 36.852 |
| TESTI4013742 | 0 | 0 | 0 | 0 | 0 | 0 | 100 |
| TESTI4013774 | 0 | 0 | 0 | 0 | 0 | 0 | 100 |
| TESTI4013817 | 0 | 0 | 0 | 0 | 0 | 0 | 17.756 |
| TESTI4013830 | 0 | 0 | 0 | 0 | 0 | 0 | 100 |
| TESTI4013852 | 0 | 0 | 0 | 0 | 0 | 0 | 100 |
| TESTI4013894 | 0 | 0 | 0 | 0 | 0 | 0 | 18.310 |
| TESTI4013924 | 0 | 0 | 0 | 0 | 0 | 0 | 100 |
| TESTI4013960 | 0 | 0 | 0 | 0 | 0 | 0 | 100 |
| TESTI4013962 | 0 | 0 | 0 | 0 | 0 | 0 | 100 |
| TESTI4014159 | 0 | 0 | 0 | 0 | 0 | 0 | 7.662 |
| TESTI4014175 | 0 | 0 | 0 | 0 | 0 | 0 | 17.462 |
| TESTI4014265 | 0 | 0 | 0 | 0 | 0 | 0 | 100 |
| TESTI4014276 | 0 | 0 | 0 | 0 | 0 | 0 | 100 |
| TESTI4014306 | 0 | 0 | 0 | 0 | 0 | 0 | 47.943 |
| TESTI4014392 | 0 | 0 | 0 | 0 | 0 | 0 | 100 |
| TESTI4014415 | 0 | 0 | 0 | 0 | 0 | 0 | 100 |
| TESTI4014445 | 0 | 0 | 0 | 0 | 0 | 0 | 100 |
| TESTI4014553 | 0 | 0 | 0 | 0 | 0 | 0 | 31.530 |
| TESTI4014661 | 0 | 0 | 0 | 0 | 0 | 0 | 100 |
| TESTI4014694 | 0 | 0 | 0 | 0 | 0 | 0 | 0.728 |
| TESTI4014801 | 0 | 0 | 0 | 0 | 0 | 0 | 100 |
| TESTI4014818 | 0 | 0 | 0 | 0 | 0 | 0 | 39.051 |
| TESTI4014891 | 0 | 0 | 0 | 0 | 0 | 0 | 100 |
| TESTI4014908 | 0 | 0 | 0 | 0 | 0 | 0 | 23.566 |
| TESTI4014924 | 0 | 0 | 0 | 0 | 0 | 0 | 100 |
| TESTI4014932 | 0 | 0 | 0 | 0 | 0 | 0 | 100 |
| TESTI4014977 | 0 | 0 | 0 | 0 | 0 | 0 | 100 |
| TESTI4015012 | 0 | 0 | 0 | 0 | 0 | 0 | 100 |
| TESTI4015129 | 0 | 0 | 0 | 0 | 0 | 0 | 100 |
| TESTI4015145 | 0 | 0 | 0 | 0 | 0 | 0 | 100 |
| TESTI4015204 | 0 | 0 | 0 | 0 | 0 | 0 | 51.491 |
| TESTI4015263 | 0 | 0 | 0 | 0 | 0 | 0 | 100 |
| TESTI4015293 | 0 | 0 | 0 | 0 | 0 | 0 | 100 |
| TESTI4015299 | 0 | 0 | 0 | 0 | 0 | 0 | 100 |
| TESTI4015339 | 0 | 0 | 0 | 0 | 0 | 0 | 100 |
| TESTI4015398 | 0 | 0 | 0 | 0 | 0 | 0 | 100 |
| TESTI4015442 | 0 | 0 | 0 | 0 | 0 | 0 | 26.359 |
| TESTI4015471 | 0 | 0 | 0 | 0 | 0 | 0 | 100 |
| TESTI4015477 | 0 | 0 | 0 | 0 | 0 | 0 | 100 |
| TESTI4015600 | 0 | 0 | 0 | 0 | 0 | 0 | 100 |
| TESTI4015646 | 0 | 0 | 0 | 0 | 0 | 0 | 100 |
| TESTI4015681 | 0 | 0 | 0 | 0 | 0 | 0 | 100 |
| TESTI4015688 | 0 | 0 | 0 | 0 | 0 | 0 | 100 |
| TESTI4015790 | 0 | 0 | 0 | 0 | 0 | 0 | 100 |
| TESTI4016110 | 0 | 0 | 0 | 0 | 0 | 0 | 100 |
| TESTI4016238 | 0 | 0 | 0 | 0 | 0 | 0 | 100 |
| TESTI4016551 | 0 | 0 | 0 | 0 | 0 | 0 | 100 |
| TESTI4016812 | 0 | 0 | 0 | 0 | 0 | 0 | 100 |
| TESTI4016822 | 0 | 0 | 0 | 0 | 0 | 0 | 100 |
| TESTI4016865 | 0 | 0 | 0 | 0 | 0 | 0 | 100 |
| TESTI4016882 | 0 | 0 | 0 | 0 | 0 | 0 | 100 |
| TESTI4016925 | 0 | 0 | 0 | 0 | 0 | 0 | 22.588 |
| TESTI4017001 | 0 | 0 | 0 | 0 | 0 | 0 | 100 |
| TESTI4017116 | 0 | 0 | 0 | 0 | 0 | 0 | 100 |
| TESTI4017137 | 0 | 0 | 0 | 0 | 0 | 0 | 100 |
| TESTI4017229 | 0 | 0 | 0 | 0 | 0 | 0 | 100 |
| TESTI4017254 | 0 | 0 | 0 | 0 | 0 | 0 | 100 |
| TESTI4017269 | 0 | 0 | 0 | 0 | 0 | 0 | 100 |
| TESTI4017380 | 0 | 0 | 0 | 0 | 0 | 0 | 100 |
| TESTI4017382 | 0 | 0 | 0 | 0 | 0 | 0 | 100 |
| TESTI4017543 | 0 | 0 | 0 | 0 | 0 | 0 | 100 |
| TESTI4017575 | 0 | 0 | 0 | 0 | 0 | 0 | 100 |
| TESTI4017647 | 0 | 0 | 0 | 0 | 0 | 0 | 100 |
| TESTI4017714 | 0 | 0 | 0 | 0 | 0 | 0 | 51.276 |
| TESTI4017763 | 0 | 0 | 0 | 0 | 0 | 0 | 100 |
| TESTI4017848 | 0 | 0 | 0 | 0 | 0 | 0 | 100 |
| TESTI4017854 | 0 | 0 | 91.498 | 0 | 0 | 0 | 8.502 |
| TESTI4017901 | 0 | 0 | 0 | 0 | 0 | 0 | 100 |
| TESTI4017961 | 0 | 0 | 0 | 0 | 0 | 0 | 100 |
| TESTI4017984 | 0 | 0 | 0 | 0 | 0 | 0 | 64.813 |
| TESTI4018101 | 0 | 0 | 0 | 0 | 0 | 0 | 100 |
| TESTI4018152 | 0 | 0 | 0 | 0 | 0 | 0 | 28.159 |
| TESTI4018208 | 0 | 0 | 0 | 0 | 0 | 0 | 100 |
| TESTI4018382 | 0 | 0 | 0 | 0 | 0 | 0 | 100 |
| TESTI4018436 | 0 | 0 | 0 | 0 | 0 | 0 | 3.402 |
| TESTI4018506 | 0 | 0 | 0 | 0 | 0 | 0 | 100 |
| TESTI4018555 | 0 | 0 | 0 | 0 | 0 | 0 | 100 |
| TESTI4018751 | 0 | 0 | 0 | 0 | 0 | 0 | 100 |
| TESTI4018806 | 0 | 0 | 0 | 0 | 0 | 0 | 100 |
| TESTI4018835 | 0 | 0 | 0 | 0 | 0 | 0 | 100 |
| TESTI4018859 | 0 | 0 | 0 | 0 | 0 | 0 | 100 |
| TESTI4018881 | 0 | 0 | 0 | 0 | 0 | 0 | 100 |
| TESTI4018886 | 0 | 0 | 0 | 0 | 0 | 0 | 35.404 |
| TESTI4018938 | 0 | 0 | 0 | 0 | 0 | 0 | 45.274 |
| TESTI4019140 | 0 | 0 | 0 | 0 | 0 | 0 | 100 |
| TESTI4019149 | 0 | 0 | 0 | 0 | 0 | 0 | 100 |
| TESTI4019299 | 0 | 0 | 0 | 0 | 0 | 0 | 100 |
| TESTI4019417 | 0 | 0 | 0 | 0 | 0 | 0 | 100 |
| TESTI4019440 | 0 | 0 | 0 | 0 | 0 | 0 | 100 |
| TESTI4019566 | 0 | 0 | 0 | 0 | 0 | 0 | 100 |
| TESTI4019657 | 0 | 0 | 0 | 0 | 0 | 0 | 100 |
| TESTI4019756 | 0 | 0 | 0 | 0 | 0 | 0 | 100 |
| TESTI4019843 | 0 | 0 | 0 | 0 | 0 | 0 | 26.135 |
| TESTI4020092 | 0 | 0 | 0 | 0 | 0 | 0 | 100 |
| TESTI4020102 | 0 | 0 | 0 | 0 | 0 | 0 | 100 |
| TESTI4020342 | 0 | 0 | 0 | 0 | 0 | 0 | 100 |
| TESTI4020460 | 0 | 0 | 0 | 0 | 0 | 0 | 100 |
| TESTI4020596 | 0 | 0 | 0 | 0 | 0 | 0 | 34.478 |
| TESTI4020806 | 0 | 0 | 0 | 0 | 0 | 0 | 100 |
| TESTI4020819 | 0 | 0 | 0 | 0 | 0 | 0 | 100 |
| TESTI4020920 | 0 | 0 | 0 | 0 | 0 | 0 | 100 |
| TESTI4021129 | 0 | 0 | 0 | 0 | 0 | 0 | 100 |
| TESTI4021197 | 0 | 0 | 0 | 0 | 0 | 0 | 100 |
| TESTI4021294 | 0 | 0 | 0 | 0 | 0 | 0 | 100 |
| TESTI4021377 | 0 | 0 | 0 | 0 | 0 | 0 | 100 |
| TESTI4021456 | 0 | 0 | 0 | 0 | 0 | 0 | 100 |
| TESTI4021491 | 0 | 0 | 0 | 0 | 0 | 0 | 100 |
| TESTI4021569 | 0 | 0 | 0 | 0 | 0 | 0 | 100 |
| TESTI4021713 | 0 | 0 | 0 | 0 | 0 | 0 | 100 |
| TESTI4021821 | 0 | 0 | 0 | 0 | 0 | 0 | 100 |
| TESTI4022158 | 0 | 0 | 0 | 0 | 0 | 0 | 8.724 |
| TESTI4022577 | 0 | 0 | 0 | 0 | 0 | 0 | 100 |
| TESTI4022716 | 0 | 0 | 0 | 0 | 0 | 0 | 100 |
| TESTI4022873 | 0 | 0 | 0 | 0 | 0 | 0 | 100 |
| TESTI4023096 | 0 | 0 | 0 | 0 | 0 | 0 | 100 |
| TESTI4023172 | 0 | 0 | 0 | 0 | 0 | 0 | 100 |
| TESTI4023546 | 0 | 0 | 0 | 0 | 0 | 0 | 100 |
| TESTI4023555 | 0 | 0 | 0 | 0 | 0 | 0 | 100 |
| TESTI4023654 | 0 | 0 | 0 | 0 | 0 | 0 | 100 |
| TESTI4023722 | 0 | 0 | 0 | 0 | 0 | 0 | 100 |
| TESTI4023762 | 0 | 0 | 0 | 0 | 0 | 0 | 100 |
| TESTI4023942 | 0 | 0 | 0 | 0 | 0 | 0 | 100 |
| TESTI4024096 | 0 | 0 | 0 | 0 | 0 | 0 | 100 |
| TESTI4024240 | 0 | 0 | 0 | 0 | 0 | 0 | 100 |
| TESTI4024245 | 0 | 0 | 0 | 0 | 0 | 0 | 22.035 |
| TESTI4024294 | 0 | 0 | 0 | 0 | 0 | 0 | 100 |
| TESTI4024344 | 0 | 0 | 0 | 0 | 0 | 0 | 100 |
| TESTI4024387 | 0 | 0 | 0 | 0 | 0 | 0 | 100 |
| TESTI4024420 | 0 | 0 | 0 | 0 | 0 | 0 | 24.447 |
| TESTI4024494 | 0 | 0 | 0 | 0 | 0 | 0 | 100 |
| TESTI4024740 | 0 | 0 | 0 | 0 | 0 | 0 | 23.518 |
| TESTI4024874 | 0 | 0 | 0 | 0 | 0 | 0 | 100 |
| TESTI4024890 | 0 | 0 | 0 | 0 | 0 | 0 | 100 |
| TESTI4024907 | 0 | 0 | 0 | 0 | 0 | 0 | 100 |
| TESTI4024930 | 0 | 0 | 0 | 0 | 0 | 0 | 100 |
| TESTI4025062 | 0 | 0 | 0 | 0 | 0 | 0 | 100 |
| TESTI4025268 | 0 | 0 | 0 | 0 | 0 | 0 | 39.529 |
| TESTI4025401 | 0 | 0 | 0 | 0 | 0 | 0 | 100 |
| TESTI4025494 | 0 | 0 | 0 | 0 | 0 | 0 | 100 |
| TESTI4025547 | 0 | 0 | 0 | 0 | 0 | 0 | 39.051 |
| TESTI4025731 | 0 | 0 | 0 | 0 | 0 | 0 | 100 |
| TESTI4025797 | 0 | 0 | 0 | 0 | 0 | 0 | 100 |
| TESTI4025865 | 0 | 0 | 0 | 0 | 0 | 0 | 100 |
| TESTI4025908 | 0 | 0 | 0 | 0 | 0 | 0 | 100 |
| TESTI4025920 | 0 | 0 | 0 | 0 | 0 | 0 | 100 |
| TESTI4026079 | 54.872 | 0 | 0 | 0 | 0 | 0 | 16.630 |
| TESTI4026080 | 0 | 0 | 0 | 0 | 0 | 0 | 100 |
| TESTI4026192 | 0 | 0 | 0 | 0 | 0 | 0 | 100 |
| TESTI4026207 | 0 | 0 | 0 | 0 | 0 | 0 | 39.051 |
| TESTI4026295 | 0 | 0 | 0 | 0 | 0 | 0 | 100 |
| TESTI4026390 | 0 | 0 | 0 | 0 | 0 | 0 | 47.943 |
| TESTI4026456 | 0 | 0 | 0 | 0 | 0 | 0 | 100 |
| TESTI4026510 | 0 | 0 | 0 | 0 | 0 | 0 | 100 |
| TESTI4026524 | 0 | 0 | 0 | 0 | 0 | 0 | 100 |
| TESTI4026680 | 0 | 0 | 0 | 0 | 0 | 0 | 100 |
| TESTI4026700 | 0 | 0 | 0 | 0 | 0 | 0 | 100 |
| TESTI4026762 | 0 | 0 | 0 | 0 | 0 | 0 | 100 |
| TESTI4026785 | 0 | 0 | 0 | 0 | 0 | 0 | 100 |
| TESTI4027139 | 0 | 0 | 0 | 0 | 0 | 0 | 100 |
| TESTI4027170 | 0 | 0 | 0 | 0 | 0 | 0 | 100 |
| TESTI4027262 | 0 | 0 | 0 | 0 | 0 | 0 | 100 |
| TESTI4027516 | 0 | 0 | 0 | 0 | 0 | 0 | 100 |
| TESTI4027557 | 0 | 0 | 0 | 0 | 0 | 0 | 100 |
| TESTI4027660 | 0 | 0 | 0 | 0 | 0 | 0 | 100 |
| TESTI4027821 | 0 | 0 | 0 | 0 | 0 | 0 | 39.529 |
| TESTI4027969 | 0 | 0 | 0 | 0 | 0 | 0 | 100 |
| TESTI4028042 | 0 | 0 | 0 | 0 | 0 | 0 | 100 |
| TESTI4028059 | 0 | 0 | 0 | 0 | 0 | 0 | 100 |
| TESTI4028062 | 0 | 0 | 0 | 0 | 0 | 0 | 100 |
| TESTI4028182 | 0 | 0 | 0 | 0 | 0 | 0 | 100 |
| TESTI4028429 | 0 | 0 | 0 | 0 | 0 | 0 | 100 |
| TESTI4028612 | 0 | 0 | 0 | 0 | 0 | 0 | 100 |
| TESTI4028692 | 0 | 0 | 0 | 0 | 0 | 0 | 100 |
| TESTI4028809 | 0 | 0 | 0 | 0 | 0 | 0 | 100 |
| TESTI4028823 | 0 | 0 | 0 | 0 | 0 | 0 | 100 |
| TESTI4028880 | 0 | 0 | 0 | 0 | 0 | 0 | 100 |
| TESTI4028904 | 0 | 0 | 0 | 0 | 0 | 0 | 100 |
| TESTI4028938 | 0 | 0 | 0 | 0 | 0 | 0 | 100 |
| TESTI4028958 | 0 | 0 | 0 | 0 | 0 | 0 | 100 |
| TESTI4028983 | 0 | 0 | 0 | 0 | 0 | 0 | 100 |
| TESTI4029023 | 0 | 0 | 0 | 0 | 0 | 0 | 100 |
| TESTI4029348 | 0 | 0 | 0 | 0 | 0 | 0 | 100 |
| TESTI4029370 | 0 | 0 | 0 | 0 | 0 | 0 | 100 |
| TESTI4029528 | 0 | 0 | 0 | 0 | 0 | 0 | 100 |
| TESTI4029651 | 0 | 0 | 0 | 0 | 0 | 0 | 100 |
| TESTI4029671 | 0 | 0 | 0 | 0 | 0 | 0 | 12.158 |
| TESTI4029676 | 0 | 0 | 0 | 0 | 0 | 0 | 100 |
| TESTI4029690 | 0 | 0 | 0 | 0 | 0 | 0 | 100 |
| TESTI4029731 | 0 | 0 | 0 | 0 | 0 | 0 | 100 |
| TESTI4029743 | 0 | 0 | 0 | 0 | 0 | 0 | 100 |
| TESTI4030069 | 0 | 0 | 0 | 0 | 0 | 0 | 100 |
| TESTI4030159 | 0 | 0 | 0 | 0 | 0 | 0 | 100 |
| TESTI4030319 | 0 | 0 | 0 | 0 | 0 | 0 | 100 |
| TESTI4030505 | 0 | 0 | 0 | 0 | 0 | 0 | 100 |
| TESTI4030603 | 0 | 0 | 0 | 0 | 0 | 0 | 100 |
| TESTI4030669 | 0 | 0 | 0 | 0 | 0 | 0 | 100 |
| TESTI4030673 | 0 | 0 | 0 | 0 | 0 | 0 | 100 |
| TESTI4030864 | 0 | 0 | 0 | 0 | 0 | 0 | 100 |
| TESTI4031066 | 0 | 0 | 0 | 0 | 0 | 0 | 100 |
| TESTI4031173 | 0 | 0 | 0 | 0 | 0 | 0 | 100 |
| TESTI4031335 | 0 | 0 | 0 | 0 | 0 | 0 | 100 |
| TESTI4031745 | 0 | 0 | 0 | 0 | 0 | 0 | 100 |
| TESTI4031818 | 0 | 0 | 0 | 0 | 0 | 0 | 100 |
| TESTI4032090 | 0 | 0 | 0 | 0 | 0 | 0 | 39.529 |
| TESTI4032112 | 0 | 0 | 0 | 0 | 0 | 0 | 100 |
| TESTI4032128 | 0 | 0 | 0 | 0 | 0 | 0 | 100 |
| TESTI4032270 | 0 | 0 | 0 | 0 | 0 | 0 | 100 |
| TESTI4032375 | 0 | 0 | 0 | 0 | 0 | 0 | 100 |
| TESTI4032834 | 0 | 0 | 0 | 0 | 0 | 0 | 100 |
| TESTI4032856 | 0 | 0 | 0 | 0 | 0 | 0 | 100 |
| TESTI4032895 | 0 | 0 | 0 | 0 | 0 | 0 | 100 |
| TESTI4032913 | 0 | 0 | 0 | 0 | 0 | 0 | 10.597 |
| TESTI4033146 | 0 | 0 | 0 | 0 | 0 | 0 | 100 |
| TESTI4033177 | 0 | 0 | 0 | 0 | 0 | 0 | 100 |
| TESTI4033433 | 0 | 0 | 0 | 0 | 0 | 0 | 100 |
| TESTI4033690 | 0 | 0 | 0 | 0 | 0 | 0 | 36.852 |
| TESTI4034172 | 0 | 0 | 0 | 0 | 0 | 0 | 100 |
| TESTI4034212 | 0 | 0 | 0 | 0 | 0 | 0 | 100 |
| TESTI4034432 | 0 | 0 | 0 | 0 | 0 | 0 | 100 |
| TESTI4034495 | 0 | 0 | 0 | 0 | 0 | 0 | 100 |
| TESTI4034632 | 0 | 0 | 0 | 0 | 0 | 0 | 100 |
| TESTI4034633 | 0 | 0 | 0 | 0 | 0 | 0 | 100 |
| TESTI4034912 | 0 | 0 | 0 | 0 | 0 | 0 | 100 |
| TESTI4034973 | 0 | 0 | 0 | 0 | 0 | 0 | 100 |
| TESTI4035063 | 0 | 0 | 0 | 0 | 0 | 0 | 100 |
| TESTI4035065 | 0 | 0 | 0 | 0 | 0 | 0 | 100 |
| TESTI4035498 | 0 | 0 | 0 | 0 | 0 | 0 | 100 |
| TESTI4035508 | 0 | 0 | 0 | 0 | 0 | 0 | 100 |
| TESTI4035581 | 0 | 0 | 0 | 0 | 0 | 0 | 100 |
| TESTI4035602 | 0 | 0 | 0 | 0 | 0 | 0 | 100 |
| TESTI4035637 | 0 | 0 | 0 | 0 | 0 | 0 | 100 |
| TESTI4035649 | 0 | 0 | 0 | 0 | 0 | 0 | 100 |
| TESTI4035770 | 0 | 0 | 0 | 0 | 0 | 0 | 34.478 |
| TESTI4035872 | 0 | 0 | 0 | 0 | 0 | 0 | 100 |
| TESTI4035895 | 0 | 0 | 0 | 0 | 0 | 0 | 100 |
| TESTI4035898 | 0 | 0 | 0 | 0 | 0 | 0 | 100 |
| TESTI4035989 | 0 | 0 | 0 | 0 | 0 | 0 | 100 |
| TESTI4036012 | 0 | 0 | 0 | 0 | 0 | 0 | 100 |
| TESTI4036042 | 0 | 0 | 0 | 0 | 0 | 0 | 100 |
| TESTI4036048 | 0 | 0 | 0 | 0 | 0 | 0 | 100 |
| TESTI4036315 | 0 | 0 | 0 | 0 | 0 | 0 | 100 |
| TESTI4036319 | 0 | 0 | 0 | 0 | 0 | 0 | 100 |
| TESTI4036449 | 0 | 0 | 0 | 0 | 0 | 0 | 100 |
| TESTI4036767 | 0 | 0 | 0 | 0 | 0 | 0 | 100 |
| TESTI4036909 | 0 | 0 | 0 | 0 | 0 | 0 | 100 |
| TESTI4037066 | 0 | 0 | 0 | 0 | 0 | 0 | 100 |
| TESTI4037156 | 0 | 0 | 3.893 | 5.840 | 0 | 0 | 0.904 |
| TESTI4037188 | 0 | 0 | 0 | 0 | 0 | 0 | 39.348 |
| TESTI4037228 | 0 | 0 | 0 | 0 | 0 | 0 | 100 |
| TESTI4037244 | 0 | 0 | 0 | 0 | 0 | 0 | 100 |
| TESTI4037727 | 0 | 0 | 0 | 0 | 0 | 0 | 100 |
| TESTI4037949 | 0 | 0 | 0 | 0 | 0 | 0 | 100 |
| TESTI4038047 | 0 | 0 | 0 | 0 | 0 | 0 | 100 |
| TESTI4038099 | 0 | 0 | 0 | 0 | 0 | 0 | 43.258 |
| TESTI4038156 | 0 | 0 | 0 | 0 | 0 | 0 | 100 |
| TESTI4038223 | 0 | 0 | 0 | 0 | 0 | 0 | 100 |
| TESTI4038258 | 0 | 0 | 0 | 0 | 0 | 0 | 100 |
| TESTI4038284 | 0 | 0 | 0 | 0 | 0 | 0 | 7.223 |
| TESTI4038339 | 0 | 0 | 0 | 0 | 0 | 0 | 100 |
| TESTI4038492 | 0 | 0 | 0 | 0 | 0 | 0 | 100 |
| TESTI4038721 | 0 | 0 | 0 | 0 | 0 | 0 | 100 |
| TESTI4038758 | 0 | 0 | 0 | 0 | 0 | 0 | 100 |
| TESTI4038779 | 0 | 0 | 0 | 0 | 0 | 0 | 100 |
| TESTI4038818 | 0 | 0 | 0 | 0 | 0 | 0 | 100 |
| TESTI4039038 | 0 | 0 | 0 | 0 | 0 | 0 | 100 |
| TESTI4039086 | 0 | 0 | 0 | 0 | 0 | 0 | 100 |
| TESTI4039451 | 0 | 0 | 0 | 0 | 0 | 0 | 100 |
| TESTI4039575 | 0 | 0 | 0 | 0 | 0 | 0 | 100 |
| TESTI4039659 | 0 | 0 | 0 | 0 | 0 | 0 | 100 |
| TESTI4039744 | 0 | 0 | 0 | 0 | 0 | 0 | 100 |
| TESTI4039904 | 0 | 0 | 0 | 0 | 0 | 0 | 100 |
| TESTI4040197 | 0 | 0 | 0 | 0 | 0 | 0 | 100 |
| TESTI4040363 | 0 | 0 | 0 | 0 | 0 | 0 | 100 |
| TESTI4040535 | 0 | 0 | 0 | 0 | 0 | 0 | 100 |
| TESTI4040559 | 0 | 0 | 0 | 0 | 0 | 0 | 100 |
| TESTI4040598 | 0 | 0 | 0 | 0 | 0 | 0 | 2.798 |
| TESTI4040800 | 0 | 0 | 0 | 0 | 0 | 0 | 100 |
| TESTI4040804 | 0 | 0 | 0 | 0 | 0 | 0 | 100 |
| TESTI4040939 | 0 | 0 | 0 | 0 | 0 | 0 | 100 |
| TESTI4040956 | 0 | 0 | 0 | 0 | 0 | 0 | 100 |
| TESTI4041049 | 0 | 0 | 0 | 0 | 0 | 0 | 100 |
| TESTI4041053 | 0 | 0 | 0 | 0 | 0 | 0 | 100 |
| TESTI4041086 | 0 | 0 | 0 | 0 | 0 | 0 | 100 |
| TESTI4041099 | 0 | 0 | 0 | 0 | 0 | 0 | 100 |
| TESTI4041143 | 0 | 0 | 0 | 0 | 0 | 0 | 100 |
| TESTI4041482 | 0 | 0 | 0 | 0 | 0 | 0 | 100 |
| TESTI4041519 | 0 | 0 | 0 | 0 | 0 | 0 | 100 |
| TESTI4041624 | 0 | 0 | 0 | 0 | 0 | 0 | 100 |
| TESTI4041629 | 0 | 0 | 0 | 0 | 0 | 0 | 100 |
| TESTI4041832 | 0 | 0 | 0 | 0 | 0 | 0 | 100 |
| TESTI4041844 | 0 | 0 | 0 | 0 | 0 | 0 | 100 |
| TESTI4041903 | 0 | 0 | 0 | 0 | 0 | 0 | 100 |
| TESTI4041954 | 0 | 0 | 0 | 0 | 0 | 0 | 100 |
| TESTI4041984 | 0 | 0 | 0 | 0 | 0 | 0 | 100 |
| TESTI4041985 | 0 | 0 | 0 | 0 | 0 | 0 | 100 |
| TESTI4041986 | 0 | 0 | 0 | 0 | 0 | 0 | 100 |
| TESTI4042098 | 0 | 0 | 0 | 0 | 0 | 0 | 100 |
| TESTI4042420 | 0 | 0 | 0 | 0 | 0 | 0 | 100 |
| TESTI4042440 | 0 | 0 | 0 | 0 | 0 | 0 | 100 |
| TESTI4042444 | 0 | 0 | 0 | 0 | 0 | 0 | 100 |
| TESTI4042711 | 0 | 0 | 0 | 0 | 0 | 0 | 100 |
| TESTI4042846 | 0 | 0 | 0 | 0 | 0 | 0 | 100 |
| TESTI4043067 | 0 | 0 | 0 | 0 | 0 | 0 | 100 |
| TESTI4043129 | 0 | 0 | 0 | 0 | 0 | 0 | 100 |
| TESTI4043140 | 0 | 0 | 0 | 0 | 0 | 0 | 5.438 |
| TESTI4043166 | 0 | 0 | 0 | 0 | 0 | 0 | 100 |
| TESTI4043203 | 0 | 0 | 0 | 0 | 0 | 0 | 100 |
| TESTI4043223 | 0 | 0 | 0 | 0 | 0 | 0 | 18.091 |
| TESTI4043371 | 0 | 0 | 0 | 0 | 0 | 0 | 100 |
| TESTI4043378 | 0 | 0 | 0 | 0 | 0 | 0 | 100 |
| TESTI4043512 | 0 | 0 | 0 | 0 | 0 | 0 | 100 |
| TESTI4043551 | 0 | 0 | 0 | 0 | 0 | 0 | 100 |
| TESTI4043710 | 0 | 0 | 0 | 0 | 0 | 0 | 100 |
| TESTI4043947 | 0 | 0 | 0 | 0 | 0 | 0 | 100 |
| TESTI4044035 | 0 | 0 | 0 | 0 | 0 | 0 | 100 |
| TESTI4044076 | 0 | 0 | 0 | 0 | 0 | 0 | 100 |
| TESTI4044084 | 0 | 0 | 0 | 0 | 0 | 0 | 100 |
| TESTI4044123 | 0 | 0 | 0 | 0 | 0 | 0 | 100 |
| TESTI4044186 | 0 | 0 | 0 | 0 | 0 | 0 | 100 |
| TESTI4044234 | 0 | 0 | 0 | 0 | 0 | 0 | 100 |
| TESTI4044291 | 0 | 0 | 0 | 0 | 0 | 0 | 100 |
| TESTI4044296 | 0 | 0 | 0 | 0 | 0 | 0 | 100 |
| TESTI4044300 | 0 | 0 | 0 | 0 | 0 | 0 | 100 |
| TESTI4044682 | 0 | 0 | 0 | 0 | 0 | 0 | 100 |
| TESTI4044704 | 0 | 0 | 0 | 0 | 0 | 0 | 3.332 |
| TESTI4044770 | 0 | 0 | 0 | 0 | 0 | 0 | 100 |
| TESTI4045168 | 0 | 0 | 0 | 0 | 0 | 0 | 100 |
| TESTI4045312 | 0 | 0 | 0 | 0 | 0 | 0 | 100 |
| TESTI4045330 | 0 | 0 | 0 | 0 | 0 | 0 | 100 |
| TESTI4045470 | 0 | 0 | 0 | 0 | 0 | 0 | 100 |
| TESTI4045701 | 0 | 0 | 0 | 0 | 0 | 0 | 100 |
| TESTI4045908 | 0 | 0 | 0 | 0 | 0 | 0 | 100 |
| TESTI4045955 | 0 | 0 | 0 | 0 | 0 | 0 | 100 |
| TESTI4045982 | 0 | 0 | 0 | 0 | 0 | 0 | 100 |
| TESTI4046073 | 0 | 0 | 0 | 0 | 0 | 0 | 12.411 |
| TESTI4046090 | 0 | 0 | 0 | 0 | 0 | 0 | 100 |
| TESTI4046240 | 0 | 0 | 0 | 0 | 0 | 0 | 100 |
| TESTI4046245 | 0 | 0 | 0 | 0 | 0 | 0 | 100 |
| TESTI4046253 | 0 | 0 | 0 | 0 | 0 | 0 | 100 |
| TESTI4046282 | 0 | 0 | 0 | 0 | 0 | 0 | 100 |
| TESTI4046328 | 0 | 0 | 0 | 0 | 0 | 0 | 100 |
| TESTI4046450 | 0 | 0 | 0 | 0 | 0 | 0 | 100 |
| TESTI4046457 | 0 | 0 | 0 | 0 | 0 | 0 | 100 |
| TESTI4046487 | 0 | 0 | 0 | 0 | 0 | 0 | 100 |
| TESTI4046502 | 0 | 0 | 0 | 0 | 0 | 0 | 56.661 |
| TESTI4046523 | 0 | 0 | 0 | 0 | 0 | 0 | 100 |
| TESTI4046819 | 0 | 0 | 0 | 0 | 0 | 0 | 12.906 |
| TESTI4046873 | 0 | 0 | 0 | 0 | 0 | 0 | 26.863 |
| TESTI4046884 | 0 | 0 | 0 | 0 | 0 | 0 | 100 |
| TESTI4046896 | 0 | 0 | 0 | 0 | 0 | 0 | 100 |
| TESTI4046903 | 0 | 0 | 0 | 0 | 0 | 0 | 100 |
| TESTI4046962 | 0 | 0 | 0 | 0 | 0 | 0 | 100 |
| TESTI4047069 | 0 | 0 | 0 | 0 | 0 | 0 | 100 |
| TESTI4047119 | 0 | 0 | 0 | 0 | 0 | 0 | 100 |
| TESTI4047305 | 0 | 0 | 0 | 0 | 0 | 0 | 100 |
| TESTI4047328 | 0 | 0 | 0 | 0 | 0 | 0 | 100 |
| TESTI4047437 | 0 | 0 | 0 | 0 | 0 | 0 | 100 |
| TESTI4047569 | 0 | 0 | 0 | 0 | 0 | 0 | 100 |
| TESTI4047746 | 0 | 0 | 0 | 0 | 0 | 0 | 100 |
| TESTI4047808 | 0 | 0 | 0 | 0 | 0 | 0 | 100 |
| THYMU1000002 | 0 | 0 | 4.164 | 0 | 0 | 0 | 0 |
| THYMU1000016 | 0.833 | 8.583 | 0 | 0 | 0 | 2.644 | 0.758 |
| THYMU1000083 | 0 | 0 | 0 | 0 | 0 | 0 | 7.840 |
| THYMU1000109 | 1.698 | 0 | 11.073 | 0 | 16.686 | 0 | 0.514 |
| THYMU1000329 | 0 | 0 | 0 | 0 | 0 | 0 | 3.484 |
| THYMU1000331 | 0 | 0 | 0 | 0 | 0 | 0 | 29.749 |
| THYMU1000359 | 3.468 | 0 | 0 | 0 | 0 | 0 | 0 |
| THYMU1000374 | 0 | 0 | 0 | 0 | 0 | 0 | 0.979 |
| THYMU1000428 | 0 | 0 | 3.999 | 0 | 0 | 0 | 1.486 |
| THYMU1000490 | 0 | 0 | 0 | 0 | 0 | 0 | 3.748 |
| THYMU2000382 | 0 | 0 | 0 | 0 | 0 | 4.147 | 0.792 |
| THYMU2000436 | 0 | 0 | 0 | 0 | 0 | 0 | 4.216 |
| THYMU2000946 | 0 | 0 | 10.178 | 0 | 0 | 0 | 0 |
| THYMU2001007 | 11.703 | 0 | 0 | 0 | 0 | 0 | 0 |
| THYMU2001090 | 0 | 0 | 0 | 0 | 0 | 0 | 13.892 |
| THYMU2001381 | 0 | 0 | 47.714 | 0 | 0 | 0 | 0 |
| THYMU2001839 | 33.745 | 0 | 0 | 0 | 0 | 0 | 10.227 |
| THYMU2001926 | 0 | 0 | 0 | 0 | 0 | 0 | 12.687 |
| THYMU2002162 | 23.514 | 0 | 0 | 0 | 0 | 0 | 0 |
| THYMU2002583 | 2.236 | 0 | 0 | 0 | 0 | 0 | 2.371 |
| THYMU2002841 | 0 | 0 | 20.416 | 0 | 0 | 0 | 1.897 |
| THYMU2003012 | 1.890 | 0 | 0 | 0 | 0 | 0 | 0 |
| THYMU2003046 | 4.146 | 0 | 0 | 0 | 0 | 0 | 1.257 |
| THYMU2003419 | 0 | 0 | 0 | 0 | 0 | 0 | 8.207 |
| THYMU2003446 | 0 | 0 | 0 | 0 | 0 | 0 | 3.118 |
| THYMU2003499 | 0 | 0 | 0 | 0 | 0 | 0 | 9.853 |
| THYMU2003891 | 1.888 | 0 | 0 | 0 | 0 | 1.997 | 0 |
| THYMU2003932 | 0 | 0 | 0 | 0 | 0 | 0 | 12.644 |
| THYMU2003981 | 9.011 | 0 | 0 | 0 | 0 | 0 | 0.341 |
| THYMU2004152 | 2.142 | 0 | 0 | 0 | 0 | 0 | 0.649 |
| THYMU2004344 | 0 | 0 | 0 | 0 | 0 | 0 | 1.670 |
| THYMU2004410 | 4.133 | 0 | 0 | 0 | 0 | 0 | 8.767 |
| THYMU2005439 | 0 | 0 | 0 | 0 | 0 | 37.819 | 0 |
| THYMU2005482 | 0 | 0 | 0 | 0 | 0 | 0 | 8.043 |
| THYMU2005546 | 1.180 | 0 | 0 | 0 | 0 | 0 | 0 |
| THYMU2005980 | 0 | 0 | 0 | 0 | 0 | 0 | 10.601 |
| THYMU2006261 | 0 | 0 | 0 | 0 | 0 | 0 | 75.821 |
| THYMU2006813 | 3.413 | 0 | 0 | 0 | 0 | 0 | 1.034 |
| THYMU2007060 | 27.587 | 0 | 0 | 0 | 0 | 0 | 2.787 |
| THYMU2007065 | 0 | 0 | 28.526 | 0 | 0 | 0 | 5.301 |
| THYMU2007112 | 9.864 | 0 | 0 | 0 | 0 | 31.290 | 5.979 |
| THYMU2007146 | 0 | 0 | 0 | 0 | 0 | 0 | 70.165 |
| THYMU2007415 | 5.219 | 0 | 0 | 0 | 0 | 33.113 | 0 |
| THYMU2007467 | 0 | 0 | 56.830 | 0 | 0 | 0 | 0 |
| THYMU2007886 | 2.987 | 0 | 0 | 0 | 0 | 0 | 0 |
| THYMU2008321 | 0 | 0 | 0 | 0 | 0 | 0 | 1.890 |
| THYMU2008383 | 0 | 0 | 0 | 0 | 0 | 0 | 43.944 |
| THYMU2009658 | 0.756 | 7.780 | 2.464 | 0 | 0 | 0 | 0 |
| THYMU2010094 | 0 | 0 | 0 | 0 | 0 | 0 | 7.615 |
| THYMU2010448 | 0 | 0 | 0 | 0 | 0 | 36.228 | 3.461 |
| THYMU2012024 | 0 | 0 | 0 | 0 | 0 | 0 | 1.948 |
| THYMU2012625 | 0 | 0 | 0 | 0 | 0 | 7.107 | 0 |
| THYMU2012826 | 0 | 0 | 0 | 0 | 0 | 0 | 14.477 |
| THYMU2013136 | 1.764 | 0 | 0 | 0 | 0 | 0 | 0 |
| THYMU2013916 | 2.392 | 0 | 0 | 0 | 0 | 0 | 2.175 |
| THYMU2016523 | 0 | 0 | 0 | 0 | 0 | 0 | 21.566 |
| THYMU2017601 | 0 | 0 | 49.678 | 0 | 0 | 0 | 18.465 |
| THYMU2017831 | 0 | 0 | 0 | 0 | 0 | 0 | 43.944 |
| THYMU2017948 | 0 | 0 | 0 | 0 | 0 | 0 | 28.159 |
| THYMU2018126 | 0 | 0 | 0 | 0 | 0 | 4.978 | 0 |
| THYMU2027125 | 1.158 | 3.976 | 0 | 7.555 | 15.180 | 2.449 | 1.521 |
| THYMU2028739 | 0 | 0 | 0 | 0 | 0 | 0 | 43.944 |
| THYMU2031341 | 0 | 0 | 0 | 0 | 0 | 0 | 2.255 |
| THYMU2031368 | 0 | 0 | 0 | 0 | 96.216 | 0 | 0 |
| THYMU2032155 | 0 | 0 | 0 | 0 | 0 | 48.859 | 0 |
| THYMU2032696 | 0 | 0 | 0 | 0 | 0 | 0 | 0.687 |
| THYMU2035735 | 0 | 0 | 0 | 0 | 0 | 0 | 1.725 |
| THYMU2036058 | 0 | 0 | 0 | 0 | 0 | 0 | 43.944 |
| THYMU2039350 | 0 | 0 | 0 | 0 | 0 | 0 | 43.944 |
| THYMU3000776 | 0 | 0 | 0 | 0 | 0 | 0 | 10.200 |
| THYMU3003309 | 0 | 0 | 0 | 0 | 0 | 0 | 19.005 |
| THYMU3003865 | 0 | 0 | 0 | 0 | 0 | 0 | 82.467 |
| THYMU3008883 | 52.467 | 0 | 0 | 0 | 0 | 0 | 0 |
| THYMU3009643 | 0 | 0 | 0 | 0 | 0 | 0 | 79.673 |
| THYMU3012983 | 0 | 0 | 0 | 0 | 0 | 0 | 43.944 |
| THYMU3013386 | 0 | 0 | 5.266 | 0 | 0 | 0 | 1.957 |
| THYMU3015759 | 2.428 | 0 | 0 | 0 | 0 | 0 | 0 |
| THYMU3021586 | 0 | 0 | 0 | 0 | 0 | 0 | 8.100 |
| THYMU3032798 | 4.171 | 0 | 0 | 0 | 0 | 0 | 0 |
| THYMU3033626 | 0 | 0 | 0 | 0 | 0 | 0 | 7.348 |
| THYMU3034671 | 5.144 | 0 | 0 | 0 | 0 | 0 | 0 |
| THYMU3041428 | 0 | 0 | 0 | 0 | 0 | 0 | 0.167 |
| THYMU3047115 | 0 | 0 | 0 | 0 | 0 | 0 | 0.269 |
| TKIDN2000701 | 0 | 0 | 0 | 0 | 0 | 0 | 15.009 |
| TKIDN2005487 | 0 | 0 | 5.054 | 0 | 0 | 4.915 | 0 |
| TKIDN2015423 | 0 | 0 | 0 | 0 | 0 | 0 | 0.832 |
| TLIVE2004110 | 22.269 | 0 | 0 | 0 | 0 | 0 | 6.749 |
| TLIVE2008229 | 0 | 0 | 0 | 0 | 0 | 0 | 8.683 |
| TRACH1000030 | 0 | 0 | 0 | 0 | 0 | 0 | 0.451 |
| TRACH1000038 | 8.123 | 0 | 0 | 0 | 0 | 0 | 9.848 |
| TRACH1000063 | 0 | 0 | 0 | 0 | 0 | 14.715 | 4.218 |
| TRACH1000193 | 0 | 0 | 0 | 0 | 0 | 0 | 14.199 |
| TRACH1000205 | 0 | 0 | 0 | 0 | 0 | 0 | 2.572 |
| TRACH2000079 | 2.337 | 12.032 | 0 | 0 | 0 | 3.706 | 0 |
| TRACH2000237 | 0 | 0 | 0 | 0 | 0 | 0 | 3.515 |
| TRACH2000248 | 3.010 | 0 | 0 | 0 | 0 | 0 | 0 |
| TRACH2000306 | 12.199 | 0 | 0 | 0 | 0 | 0 | 3.697 |
| TRACH2000321 | 1.060 | 0 | 3.456 | 0 | 0 | 0 | 0.963 |
| TRACH2000359 | 0 | 0 | 20.978 | 0 | 0 | 0 | 0 |
| TRACH2000420 | 0 | 0 | 0 | 0 | 0 | 0 | 2.192 |
| TRACH2000461 | 0 | 0 | 0 | 0 | 0 | 0 | 11.208 |
| TRACH2000540 | 3.073 | 0 | 0 | 0 | 0 | 0 | 0 |
| TRACH2000702 | 0 | 0 | 0 | 0 | 0 | 0 | 2.566 |
| TRACH2000780 | 5.655 | 0 | 0 | 0 | 0 | 0 | 7.999 |
| TRACH2000862 | 0 | 0 | 0 | 0 | 0 | 39.606 | 0 |
| TRACH2000920 | 0 | 0 | 0 | 0 | 0 | 0 | 4.334 |
| TRACH2000926 | 0 | 0 | 19.627 | 0 | 0 | 19.089 | 0 |
| TRACH2000959 | 0 | 0 | 0 | 0 | 0 | 0 | 0.160 |
| TRACH2000972 | 0 | 26.327 | 4.169 | 0 | 12.565 | 0 | 0.775 |
| TRACH2001021 | 0 | 0 | 0 | 0 | 0 | 2.865 | 0 |
| TRACH2001101 | 0 | 0 | 0 | 0 | 0 | 18.186 | 0 |
| TRACH2001192 | 0 | 0 | 0 | 0 | 0 | 0 | 22.035 |
| TRACH2001249 | 0 | 0 | 0 | 0 | 0 | 0 | 59.944 |
| TRACH2001289 | 0 | 0 | 0 | 0 | 0 | 0 | 0.222 |
| TRACH2001314 | 0 | 0 | 0 | 0 | 0 | 0 | 11.948 |
| TRACH2001478 | 0 | 0 | 0 | 0 | 0 | 0 | 2.277 |
| TRACH2001607 | 2.166 | 0 | 0 | 0 | 0 | 0 | 7.222 |
| TRACH2001612 | 0 | 0 | 0 | 0 | 0 | 0 | 2.611 |
| TRACH2001810 | 1.675 | 0 | 0 | 0 | 0 | 0 | 0 |
| TRACH2002537 | 0 | 0 | 6.524 | 0 | 0 | 0 | 0.606 |
| TRACH2002664 | 0 | 0 | 10.705 | 0 | 0 | 0 | 0 |
| TRACH2002803 | 0 | 0 | 0 | 0 | 0 | 0 | 5.449 |
| TRACH2003323 | 3.839 | 0 | 0 | 0 | 0 | 0 | 1.163 |
| TRACH2004039 | 0 | 0 | 0 | 0 | 0 | 0 | 36.852 |
| TRACH2004336 | 0 | 0 | 0 | 0 | 0 | 0 | 36.852 |
| TRACH2005541 | 0 | 0 | 68.404 | 0 | 0 | 0 | 0 |
| TRACH2005800 | 2.594 | 0 | 0 | 0 | 0 | 0 | 0 |
| TRACH2005811 | 0 | 0 | 0 | 0 | 0 | 14.431 | 3.447 |
| TRACH2006378 | 0 | 0 | 0 | 0 | 0 | 0 | 5.843 |
| TRACH2006866 | 0 | 0 | 0 | 0 | 21.039 | 0 | 0 |
| TRACH2007399 | 0 | 10.463 | 0 | 0 | 0 | 0 | 0.308 |
| TRACH2007969 | 0 | 0 | 0 | 0 | 0 | 0 | 4.977 |
| TRACH2008081 | 0 | 0 | 0 | 0 | 0 | 0 | 0.835 |
| TRACH2008300 | 0 | 0 | 0 | 0 | 0 | 0 | 1.110 |
| TRACH2008472 | 6.768 | 0 | 11.037 | 0 | 0 | 0 | 4.103 |
| TRACH2008716 | 0 | 0 | 0 | 0 | 0 | 34.708 | 3.316 |
| TRACH2009107 | 0 | 0 | 0 | 0 | 0 | 85.932 | 0 |
| TRACH2009310 | 0 | 0 | 0 | 0 | 0 | 0 | 80.334 |
| TRACH2009340 | 0 | 0 | 0 | 0 | 0 | 67.720 | 12.940 |
| TRACH2009661 | 0 | 0 | 0 | 0 | 0 | 0 | 36.852 |
| TRACH2011113 | 0 | 0 | 0 | 0 | 0 | 0 | 3.086 |
| TRACH2012298 | 0 | 0 | 0 | 0 | 30.085 | 0 | 0 |
| TRACH2012376 | 0 | 0 | 0 | 0 | 0 | 18.363 | 0 |
| TRACH2012387 | 0 | 0 | 0 | 0 | 0 | 0 | 36.852 |
| TRACH2012811 | 0 | 0 | 0 | 0 | 0 | 0 | 5.096 |
| TRACH2014077 | 0 | 0 | 0 | 0 | 0 | 0 | 2.224 |
| TRACH2014124 | 2.226 | 0 | 0 | 0 | 0 | 7.062 | 0 |
| TRACH2015823 | 4.718 | 0 | 0 | 0 | 0 | 0 | 0 |
| TRACH2015824 | 0 | 0 | 16.348 | 0 | 0 | 0 | 0 |
| TRACH2018446 | 9.020 | 0 | 0 | 0 | 0 | 0 | 0 |
| TRACH2018449 | 0 | 0 | 0 | 0 | 0 | 0 | 9.911 |
| TRACH2019080 | 65.818 | 0 | 0 | 0 | 0 | 0 | 0 |
| TRACH2023299 | 0 | 0 | 0 | 0 | 0 | 0 | 4.172 |
| TRACH2025535 | 1.252 | 12.895 | 0 | 0 | 0 | 0 | 3.036 |
| TRACH3002192 | 0 | 0 | 4.387 | 0 | 0 | 0 | 2.446 |
| TRACH3003379 | 0 | 0 | 0 | 0 | 0 | 0 | 15.996 |
| TRACH3003547 | 0 | 0 | 0 | 0 | 0 | 0 | 2.764 |
| TRACH3003832 | 0 | 0 | 0 | 0 | 0 | 0 | 7.937 |
| TRACH3003872 | 5.920 | 0 | 0 | 0 | 0 | 0 | 3.588 |
| TRACH3004068 | 0 | 0 | 10.026 | 0 | 0 | 0 | 0 |
| TRACH3004721 | 0 | 0 | 0 | 0 | 0 | 0 | 5.701 |
| TRACH3005211 | 0 | 0 | 0 | 0 | 0 | 0 | 12.047 |
| TRACH3005479 | 0 | 0 | 0 | 0 | 0 | 0 | 36.852 |
| TRACH3005699 | 0 | 0 | 0 | 0 | 0 | 0 | 6.274 |
| TRACH3006717 | 0 | 0 | 0 | 0 | 0 | 0 | 4.797 |
| TRACH3007479 | 0 | 0 | 0 | 0 | 0 | 0 | 0.703 |
| TRACH3007625 | 0 | 0 | 0 | 0 | 0 | 0 | 2.305 |
| TRACH3008093 | 0 | 0 | 0 | 0 | 0 | 0 | 1.601 |
| TRACH3008508 | 41.320 | 0 | 0 | 0 | 0 | 0 | 0 |
| TRACH3008629 | 0 | 0 | 0 | 0 | 0 | 0 | 2.980 |
| TRACH3009008 | 0 | 0 | 0 | 0 | 0 | 0 | 3.202 |
| TRACH3009148 | 19.345 | 0 | 0 | 0 | 0 | 0 | 0 |
| TRACH3013043 | 0 | 0 | 0 | 0 | 0 | 0 | 53.856 |
| TRACH3016805 | 0 | 0 | 31.673 | 0 | 0 | 0 | 5.886 |
| TRACH3016992 | 0 | 0 | 0 | 0 | 0 | 0 | 46.677 |
| TRACH3019290 | 0 | 0 | 0 | 0 | 0 | 0 | 18.081 |
| TRACH3026303 | 0 | 0 | 0 | 0 | 0 | 0 | 3.664 |
| TRACH3036942 | 0 | 0 | 0 | 0 | 0 | 0 | 0.638 |
| TRACH3037067 | 0 | 35.703 | 0 | 0 | 0 | 0 | 0 |
| TSTOM2000139 | 0 | 0 | 0 | 0 | 0 | 0 | 2.734 |
| TSTOM2000315 | 0 | 0 | 0 | 0 | 0 | 0 | 11.918 |
| TSTOM2000553 | 0 | 0 | 11.859 | 0 | 0 | 0 | 0 |
| TUTER1000122 | 0 | 0 | 0 | 0 | 0 | 0 | 2.134 |
| TUTER2000904 | 0 | 0 | 0 | 0 | 0 | 0 | 3.650 |
| UMVEN1000143 | 1.771 | 0 | 0 | 0 | 0 | 0 | 0 |
| UMVEN2000133 | 1.264 | 0 | 0 | 0 | 0 | 0 | 0.383 |
| UMVEN2000152 | 0 | 0 | 0 | 0 | 0 | 0 | 0.515 |
| UMVEN2000453 | 0 | 0 | 0 | 0 | 0 | 0 | 0.341 |
| UTERU1000182 | 0 | 0 | 5.706 | 0 | 0 | 0 | 0.530 |
| UTERU1000384 | 0 | 0 | 0 | 0 | 0 | 0 | 4.659 |
| UTERU2000023 | 0 | 0 | 0 | 0 | 0 | 0 | 6.692 |
| UTERU2000095 | 3.571 | 0 | 0 | 0 | 0 | 0 | 2.164 |
| UTERU2000263 | 1.105 | 0 | 0 | 0 | 0 | 0 | 0 |
| UTERU2000332 | 0 | 0 | 0 | 0 | 0 | 0 | 3.091 |
| UTERU2000424 | 0 | 0 | 0 | 0 | 0 | 0 | 0.555 |
| UTERU2000542 | 0 | 0 | 0 | 0 | 0 | 0 | 5.902 |
| UTERU2001607 | 3.182 | 0 | 0 | 0 | 0 | 0 | 2.893 |
| UTERU2002198 | 0 | 0 | 0 | 0 | 0 | 0 | 35.404 |
| UTERU2002294 | 0 | 0 | 0 | 0 | 0 | 12.420 | 0 |
| UTERU2002410 | 0 | 0 | 8.261 | 0 | 0 | 0 | 0.384 |
| UTERU2002662 | 0 | 39.520 | 0 | 0 | 0 | 0 | 0 |
| UTERU2002841 | 0 | 0 | 0 | 0 | 0 | 0 | 35.404 |
| UTERU2002993 | 0 | 0 | 0 | 0 | 0 | 0 | 35.404 |
| UTERU2003321 | 0 | 0 | 0 | 0 | 0 | 0 | 52.294 |
| UTERU2004073 | 0 | 0 | 0 | 0 | 0 | 0 | 76.682 |
| UTERU2004197 | 8.674 | 0 | 0 | 0 | 0 | 13.757 | 2.629 |
| UTERU2004664 | 0 | 0 | 11.937 | 0 | 0 | 0 | 0 |
| UTERU2004688 | 0 | 0 | 0 | 70.393 | 0 | 0 | 0 |
| UTERU2004807 | 2.167 | 0 | 0 | 0 | 0 | 0 | 0.657 |
| UTERU2005069 | 0 | 0 | 0 | 0 | 0 | 0 | 35.404 |
| UTERU2005074 | 24.492 | 0 | 0 | 0 | 0 | 0 | 7.423 |
| UTERU2005292 | 0 | 0 | 0 | 0 | 0 | 21.304 | 0 |
| UTERU2005354 | 4.718 | 0 | 0 | 0 | 0 | 0 | 5.720 |
| UTERU2005593 | 0 | 0 | 0 | 0 | 0 | 0 | 2.935 |
| UTERU2005621 | 0 | 0 | 0 | 0 | 0 | 0 | 4.420 |
| UTERU2007444 | 0 | 0 | 0 | 0 | 0 | 0 | 35.404 |
| UTERU2008085 | 0 | 0 | 0 | 0 | 7.659 | 1.854 | 0.708 |
| UTERU2008302 | 0 | 0 | 0 | 0 | 0 | 0 | 0.873 |
| UTERU2008747 | 0 | 0 | 0 | 0 | 0 | 0 | 5.777 |
| UTERU2008938 | 5.308 | 0 | 0 | 0 | 0 | 0 | 0 |
| UTERU2008962 | 0 | 0 | 0 | 0 | 0 | 0 | 35.404 |
| UTERU2009335 | 0 | 0 | 0 | 0 | 0 | 20.751 | 1.983 |
| UTERU2009435 | 0 | 0 | 0 | 0 | 0 | 0 | 3.496 |
| UTERU2010417 | 0 | 0 | 2.218 | 6.653 | 0 | 0 | 0.206 |
| UTERU2012230 | 0 | 0 | 0 | 0 | 0 | 0 | 1.150 |
| UTERU2012286 | 5.464 | 0 | 0 | 0 | 0 | 0 | 1.656 |
| UTERU2013262 | 0 | 0 | 0 | 0 | 0 | 0 | 11.996 |
| UTERU2014024 | 0 | 0 | 0 | 0 | 0 | 0 | 16.943 |
| UTERU2014167 | 0 | 0 | 0 | 19.235 | 0 | 0 | 0 |
| UTERU2014601 | 0 | 0 | 48.719 | 0 | 0 | 0 | 0 |
| UTERU2014898 | 0 | 0 | 0 | 0 | 0 | 0 | 0.995 |
| UTERU2015653 | 1.258 | 0 | 0 | 0 | 0 | 0 | 0 |
| UTERU2016426 | 27.411 | 0 | 0 | 0 | 0 | 0 | 0 |
| UTERU2016822 | 8.670 | 0 | 0 | 0 | 0 | 0 | 0 |
| UTERU2017623 | 0 | 0 | 0 | 0 | 0 | 0 | 35.404 |
| UTERU2017632 | 0 | 0 | 0 | 0 | 0 | 0 | 35.404 |
| UTERU2019096 | 4.968 | 0 | 0 | 0 | 0 | 0 | 1.506 |
| UTERU2020226 | 0 | 0 | 0 | 0 | 0 | 0 | 21.509 |
| UTERU2023175 | 0 | 0 | 0 | 0 | 0 | 10.186 | 0 |
| UTERU2025301 | 0 | 0 | 0 | 91.731 | 0 | 0 | 0 |
| UTERU2030213 | 0 | 0 | 0 | 0 | 0 | 0 | 16.103 |
| UTERU3000844 | 0 | 0 | 0 | 0 | 0 | 0 | 35.404 |
| UTERU3003660 | 0 | 0 | 0 | 0 | 0 | 0 | 4.813 |
| UTERU3005159 | 0 | 0 | 0 | 0 | 0 | 0 | 22.803 |
| UTERU3005767 | 0 | 0 | 0 | 0 | 0 | 8.143 | 0 |
| UTERU3009871 | 0 | 0 | 0 | 0 | 0 | 0 | 23.683 |
| UTERU3010409 | 0 | 0 | 0 | 0 | 0 | 0 | 2.262 |
| UTERU3015011 | 0 | 0 | 0 | 0 | 0 | 48.286 | 0 |
| UTERU3017995 | 0 | 0 | 0 | 0 | 0 | 74.828 | 0 |

Genes whose expression levels were negligibly low in each of the above-described -libraries are shown with their clone names below.

3NB691000018, 3NB691000085, 3NB691000113, 3NB691000129, 3NB691000191, 3NB692000012, 3NB692000029, 3NB692000102, 3NB692000154, 3NB692000276, 3NB692000305, 3NB692000429, 3NB692000484, 3NB692000973, 3NB692001002, 3NB692001022, 3NB692001034, 3NB692001040, 3NB692001123, 3NB692001267, 3NB692.001288, 3NB692001339, 3NB692001349, 3NB692001408, 3NB692001433, 3NB692001459, 3NB692001471, 3NB692001501, 3NB692001507, 3NB692001519, 3NB692001520, 3NB692001528, 3NB692001538, 3NB692001548, 3NB692001637, 3NB692001719, 3NB692001853, 3NB692001861, 3NB692002051, 3NB692002365, 3NB692002685, 3NB692002806, 3NB692003538, 3NB692004045, 3NB692004724, 3NB692005439, 3NB692006952, 3NB692008729, ACTVT2000380, ADIPS1000064,

ADIPS2000069, ADIPS2000088, ADIPS2000245, ADIPS2000425, ADRGL1000002, ADRGL1000033, ADRGL1000038, ADRGL1000067, ADRGL1000111, ADRGL1000144, ADRGL1000147, ADRGL1000160, ADRGL1000165, ADRGL1000182, ADRGL2000056, ADRGL2000064, ADRGL2000074, ADRGL2000085, ADRGL2000142, ADRGL2000172, ADRGL2000248, ADRGL2000261, ADRGL2000323, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002260, ADRGL2002392, ADRGL2002477, ADRGL2002679, ADRGL2002753, ADRGL2002857, ADRGL2003017,

ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004676, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007313, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535,

ADRGL2008966, ADRGL2009146, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO1000165, ASTRO2000011, ASTRO2000095, ASTRO2000191, ASTRO2000278, ASTRO2000372, ASTRO2000381, ASTRO2000403, ASTRO2000417, ASTRO2000480, ASTRO2000482, ASTRO2000533, ASTRO2000653, ASTRO2000662, ASTRO2000725, ASTRO2000801, ASTRO2000914, ASTRO2001001, ASTRO2001008, ASTRO2001088, ASTRO2001107, ASTRO2001122, ASTRO2001227, ASTRO2001249, ASTRO2001480, ASTRO2002035, ASTRO2002064, ASTRO2002202, ASTRO2002571,

ASTRO2002632, ASTRO2002659, ASTRO2002693, ASTRO2002733, ASTRO2002743, ASTRO2003461, ASTRO2003574, ASTRO2003581, ASTRO2003740, ASTRO2003925, ASTRO2003960, ASTRO2004032, ASTRO2004051, ASTRO2004177, ASTRO2004217, ASTRO2004297, ASTRO2004584, ASTRO2004751, ASTRO2004877, ASTRO2004974, ASTRO2005008, ASTRO2005081, ASTRO2005096, ASTRO2005242, ASTRO2005273, ASTRO2005343, ASTRO2005413, ASTRO2005423, ASTRO2005553, ASTRO2005557, ASTRO2005575, ASTRO2005593, ASTRO2005726, ASTRO2005863, ASTRO2005896, ASTRO2005902, ASTRO2006356, ASTRO2006475, ASTRO2006732, ASTRO2006747, ASTRO2006920, ASTRO2006952, ASTRO2007221, ASTRO2007377, ASTRO2007515, ASTRO2007578, ASTRO2007643, ASTRO2007666, ASTRO2007956, ASTRO2007962,

ASTRO2008040, ASTRO2008216, ASTRO2008239, ASTRO2008409, ASTRO2008425, ASTRO2008508, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010582, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2013124, ASTRO2013671, ASTRO2013802, ASTRO2014135, ASTRO2014174, ASTRO2014211, ASTRO2014363, ASTRO2014392, ASTRO2014498, ASTRO2014561, ASTRO2014863, ASTRO2014923, ASTRO2014961, ASTRO2015162, ASTRO2015185, ASTRO2015328, ASTRO2015987, ASTRO2016044, ASTRO2016148, ASTRO2016681,

ASTRO2016847, ASTRO2016892, ASTRO2017348, ASTRO2017627, ASTRO2018373, ASTRO2019039, ASTRO2019171, ASTRO3000154, ASTRO3000172, ASTRO3000177, ASTRO3000301, ASTRO3000474, ASTRO3000482, BEAST2000454, BGGI11000285, BGGI12000022, BGGI12000067, BGGI12000161, BGGI12000533, BGGI12000544, BGGI12000684, BGGI12000693, BGGI12001002, BGGI12001097, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987,

BLADE2002073, BLADE2002310, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004722, BLADE2004849, BLADE2005036, BLADE2005038, BLADE2005229, BLADE2005459, BLADE2005792, BLADE2006043, BLADE2006412, BLADE2006415, BLADE2006607, BLADE2006826, BLADE2006849, BLADE2006947, BLADE2007033, BLADE2007043, BLADE2007589, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479,

BLADE2009593, BNGH41000098, BNGH41000104, BNGH41000118, BNGH41000137, BNGH41000153, BNGH41000169, BNGH41000177, BNGH41000216, BNGH42000130, BNGH42000132, BNGH42000405, BNGH42000532, BNGH42000675, BNGH42000791, BNGH42000815, BNGH42000994, BNGH42001724, BNGH42002168, BNGH42002244, BNGH42002487, BNGH42002489, BNGH42003529, BNGH42003570, BNGH42003641, BNGH42004076, BNGH42004291, BNGH42004538, BNGH42005017, BNGH42005235, BNGH42005504, BNGH42006226, BNGH42006234, BNGH42007366, BNGH42007594, BNGH42007788, BNGH42008199, BNGH42008510, BNGH42008603, BNGH42008649, BNGH42008832, BNGH42008850, BNGH42008943, BNGH42009021, BRACE1000020, BRACE1000042, BRACE1000047, BRACE1000051, BRACE1000065, BRACE1000070,

BRACE1000084, BRACE1000087, BRACE1000092, BRACE1000093, BRACE1000101, BRACE1000114, BRACE1000124, BRACE1000132, BRACE1000166, BRACE1000169, BRACE1000186, BRACE1000187, BRACE1000239, BRACE1000258, BRACE1000451, BRACE1000475, BRACE1000572, BRACE2000061, BRACE2000078, BRACE2000100, BRACE2000109, BRACE2000141, BRACE2000148, BRACE2000232, BRACE2000245, BRACE2000256, BRACE2000266, BRACE2000267, BRACE2000280, BRACE2000300, BRACE2000307, BRACE2000331, BRACE2000332, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000640, BRACE2000650, BRACE2000677, BRACE2000698,

BRACE2000718, BRACE2000733, BRACE2000743, BRACE2000750, BRACE2000753, BRACE2000815, BRACE2000864, BRACE2000905, BRACE2000936, BRACE2000947, BRACE2000965, BRACE2000969, BRACE2000988, BRACE2001017, BRACE2001065, BRACE2001070, BRACE2001094, BRACE2001151, BRACE2001188, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001492, BRACE2001496, BRACE2001508, BRACE2001534, BRACE2001543, BRACE2001588, BRACE2001607, BRACE2001626, BRACE2001673, BRACE2001677, BRACE2001705, BRACE2001709, BRACE2001726,

BRACE2001733, BRACE2001737, BRACE2001742, BRACE2001777, BRACE2001779, BRACE2001834, BRACE2001855, BRACE2001859, BRACE2001865, BRACE2001872, BRACE2001881, BRACE2001898, BRACE2001923, BRACE2001942, BRACE2001944, BRACE2001954, BRACE2001971, BRACE2001972, BRACE2001973, BRACE2002026, BRACE2002034, BRACE2002081, BRACE2002139, BRACE2002142, BRACE2002151, BRACE2002157, BRACE2002176, BRACE2002206, BRACE2002227, BRACE2002260, BRACE2002297, BRACE2002304, BRACE2002394, BRACE2002409, BRACE2002431, BRACE2002444, BRACE2002458, BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623,

BRACE2002635, BRACE2002676, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002812, BRACE2002834, BRACE2002849, BRACE2002857, BRACE2002860, BRACE2002884, BRACE2002896, BRACE2002948, BRACE2003037, BRACE2003078, BRACE2003097, BRACE2003110, BRACE2003168, BRACE2003199, BRACE2003210, BRACE2003217, BRACE2003254, BRACE2003269, BRACE2003285, BRACE2003304, BRACE2003319, BRACE2003398, BRACE2003420, BRACE2003428, BRACE2003431, BRACE2003512, BRACE2003516, BRACE2003539, BRACE2003540, BRACE2003594, BRACE2003598, BRACE2003609, BRACE2003614, BRACE2003639, BRACE2003700, BRACE2003713,

BRACE2003766, BRACE2003785, BRACE2003788, BRACE2003800, BRACE2003802, BRACE2003825, BRACE2003845, BRACE2003847, BRACE2003848, BRACE2003873, BRACE2003885, BRACE2003887, BRACE2003905, BRACE2003928, BRACE2003944, BRACE2003954, BRACE2003982, BRACE2003987, BRACE2005034, BRACE2005047, BRACE2005063, BRACE2005083, BRACE2005099, BRACE2005118, BRACE2005124, BRACE2005138, BRACE2005160, BRACE2005169, BRACE2005189, BRACE2005193, BRACE2005196, BRACE2005198, BRACE2005216, BRACE2005243, BRACE2005251, BRACE2005253, BRACE2005290, BRACE2005298, BRACE2005328, BRACE2005329, BRACE2005337, BRACE2005348, BRACE2005363, BRACE2005395, BRACE2005408, BRACE2005448, BRACE2005450, BRACE2005453, BRACE2005457, BRACE2005460,

BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006055, BRACE2006075, BRACE2006084, BRACE2006089, BRACE2006102, BRACE2006122, BRACE2006137, BRACE2006143, BRACE2006162, BRACE2006167, BRACE2006174, BRACE2006240, BRACE2006245, BRACE2006249, BRACE2006258, BRACE2006264, BRACE2006274, BRACE2006281, BRACE2006298, BRACE2006344,

BRACE2006354, BRACE2006363, BRACE2006378, BRACE2006380, BRACE2006393, BRACE2006413, BRACE2006417, BRACE2006453, BRACE2006459, BRACE2006488, BRACE2006534, BRACE2006537, BRACE2006540, BRACE2006564, BRACE2006587, BRACE2006598, BRACE2006636, BRACE2006685, BRACE2006703, BRACE2006743, BRACE2006753, BRACE2006783, BRACE2006821, BRACE2006833, BRACE2006871, BRACE2006909, BRACE2006911, BRACE2006924, BRACE2006935, BRACE2006937, BRACE2006944, BRACE2006951, BRACE2006982, BRACE2006986, BRACE2007013, BRACE2007068, BRACE2007087, BRACE2007138, BRACE2007174, BRACE2007197, BRACE2007201, BRACE2007203, BRACE2007232, BRACE2007254, BRACE2007262, BRACE2007274, BRACE2007281, BRACE2007295, BRACE2007358, BRACE2007396,

BRACE2007398, BRACE2007411, BRACE2007447, BRACE2007477, BRACE2007502, BRACE2007503, BRACE2007522, BRACE2007527, BRACE2007538, BRACE2007563, BRACE2007577, BRACE2007621, BRACE2007640, BRACE2007641, BRACE2007646, BRACE2007663, BRACE2007671, BRACE2007682, BRACE2007685, BRACE2007727, BRACE2007749, BRACE2007760, BRACE2007761, BRACE2007764, BRACE2007767, BRACE2007768, BRACE2007769, BRACE2007784, BRACE2007786, BRACE2007798, BRACE2007799, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008097, BRACE2008102, BRACE2008114, BRACE2008144, BRACE2008172, BRACE2008216,

BRACE2008218, BRACE2008251, BRACE2008276, BRACE2008295, BRACE2008308, BRACE2008336, BRACE2008361, BRACE2008380, BRACE2008401, BRACE2008410, BRACE2008420, BRACE2008443, BRACE2008473, BRACE2008480, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008941, BRACE2008948, BRACE2008960, BRACE2008999, BRACE2009014, BRACE2009016, BRACE2009031, BRACE2009053, BRACE2009122, BRACE2009131, BRACE2009185, BRACE2009188, BRACE2009212,

BRACE2009221, BRACE2009235, BRACE2009238, BRACE2009255, BRACE2009270, BRACE2009272, BRACE2009274, BRACE2009275, BRACE2009293, BRACE2009311, BRACE2009318, BRACE2009323, BRACE2009366, BRACE2009421, BRACE2009428, BRACE2009434, BRACE2009437, BRACE2009483, BRACE2009533, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010684, BRACE2010753, BRACE2010813, BRACE2010828,

BRACE2010837, BRACE2010888, BRACE2010937, BRACE2010983, BRACE2011007, BRACE2011025, BRACE2011183, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317, BRACE2012510, BRACE2012625, BRACE2012653, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268,

BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017410, BRACE2017438, BRACE2017580, BRACE2017587, BRACE2017720, BRACE2017844, BRACE2017872, BRACE2017929, BRACE2017934,

BRACE2017985, BRACE2017992, BRACE2018337, BRACE2018443, BRACE2018448, BRACE2018568, BRACE2018700, BRACE2018727, BRACE2018759, BRACE2018762, BRACE2018782, BRACE2019004, BRACE2019044, BRACE2019147, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843, BRACE2019865, BRACE2019872, BRACE2019877, BRACE2020028, BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021148, BRACE2021245, BRACE2021254, BRACE2021264, BRACE2021344, BRACE2021433,

BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023223, BRACE2023268, BRACE2023284, BRACE2023404, BRACE2023451, BRACE2023540, BRACE2023633, BRACE2023744, BRACE2023800, BRACE2023801, BRACE2024074, BRACE2024222, BRACE2024610, BRACE2024781, BRACE2024826, BRACE2025018, BRACE2025316, BRACE2025333, BRACE2025452, BRACE2025492, BRACE2025710, BRACE2025796, BRACE2025992, BRACE2026054, BRACE2026141,

BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879, BRACE2027896, BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030345, BRACE2030736, BRACE2030883, BRACE2030884, BRACE2031154, BRACE2031232, BRACE2031527, BRACE2031531, BRACE2031553, BRACE2031899, BRACE2032044,

BRACE2032090, BRACE2032134, BRACE2032148, BRACE2032182, BRACE2032329, BRACE2032385, BRACE2032436, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299, BRACE2037310, BRACE2037431, BRACE2037692, BRACE2037847, BRACE2038114, BRACE2038271, BRACE2038329, BRACE2038492, BRACE2038551, BRACE2038618, BRACE2039009,

BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043248, BRACE2043349, BRACE2043774, BRACE2043942, BRACE2044057, BRACE2044263, BRACE2044286, BRACE2044304, BRACE2044473, BRACE2044510, BRACE2044640, BRACE2044816, BRACE2044946, BRACE2044949, BRACE2045300, BRACE2045428,

BRACE2045445, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413,

BRACE3003595, BRACE3003698, BRACE3003866, BRACE3003920, BRACE3004046, BRACE3004058, BRACE3004113, BRACE3004358, BRACE3004371, BRACE3004435, BRACE3004477, BRACE3004767, BRACE3004772, BRACE3004783, BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005760, BRACE3005870, BRACE3005903, BRACE3005989, BRACE3006113, BRACE3006185, BRACE3006226, BRACE3006462, BRACE3006463, BRACE3006872, BRACE3006917, BRACE3007084, BRACE3007085, BRACE3007258, BRACE3007322, BRACE3007472, BRACE3007480, BRACE3007559, BRACE3007625, BRACE3007642, BRACE3007649, BRACE3007767, BRACE3007869, BRACE3008036,

BRACE3008066, BRACE3008092, BRACE3008095, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009265, BRACE3009297, BRACE3009377, BRACE3009392, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780,

BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3020151, BRACE3020194, BRACE3020286, BRACE3020356, BRACE3020594, BRACE3020669, BRACE3020671, BRACE3020890, BRACE3021148, BRACE3021517,

BRACE3021805, BRACE3022051, BRACE3022303, BRACE3022312, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027478, BRACE3027480, BRACE3027931, BRACE3027976, BRACE3028214, BRACE3028241, BRACE3028360, BRACE3028998, BRACE3029005, BRACE3029021, BRACE3029205, BRACE3029447,

BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856,

BRACE3040863, BRACE3041059, BRACE3041162, BRACE3041827, BRACE3042046, BRACE3042210, BRACE3042326, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174,

BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009482, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951,

BRALZ2015168, BRALZ2015811, BRALZ2016085, BRALZ2016498, BRALZ2017359, BRALZ2017531, BRALZ2017607, BRALZ2017620, BRALZ2017844, BRALZ2018342, BRALZ2018492, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000388, BRAMY2000411, BRAMY2000508, BRAMY2000562, BRAMY2000653, BRAMY2000814, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339,

BRAMY2002364, BRAMY2002369, BRAMY2002479, BRAMY2002493, BRAMY2002525, BRAMY2002584, BRAMY2002604, BRAMY2002621, BRAMY2002638, BRAMY2002739, BRAMY2002799, BRAMY2002853, BRAMY2002862, BRAMY2002956, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004387, BRAMY2004492, BRAMY2004521, BRAMY2004524, BRAMY2004537, BRAMY2004542, BRAMY2004756, BRAMY2004771, BRAMY2005064, BRAMY2005094, BRAMY2005151, BRAMY2005182, BRAMY2005244, BRAMY2005391,

BRAMY2005576, BRAMY2005648, BRAMY2005662, BRAMY2005708, BRAMY2005711, BRAMY2006092, BRAMY2006118, BRAMY2006149, BRAMY2006366, BRAMY2006375, BRAMY2006411, BRAMY2006459, BRAMY2006466, BRAMY2006659, BRAMY2006779, BRAMY2006809, BRAMY2006879, BRAMY2007043, BRAMY2007110, BRAMY2007185, BRAMY2007244, BRAMY2007249, BRAMY2007255, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008979, BRAMY2009023, BRAMY2009034, BRAMY2009105, BRAMY2009123, BRAMY2009192, BRAMY2009344, BRAMY2009349, BRAMY2009394, BRAMY2009489, BRAMY2009508,

BRAMY2009528, BRAMY2009557, BRAMY2009612, BRAMY2009693, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011105, BRAMY2011178, BRAMY2011196, BRAMY2011216, BRAMY2011253, BRAMY2011446, BRAMY2011480, BRAMY2011639, BRAMY2011679, BRAMY2011811, BRAMY2011846, BRAMY2011849, BRAMY2011872, BRAMY2011961, BRAMY2012017, BRAMY2012091, BRAMY2012119, BRAMY2012162, BRAMY2012277, BRAMY2012340, BRAMY2012497, BRAMY2012517, BRAMY2012555, BRAMY2012568, BRAMY2012581, BRAMY2012691, BRAMY2012721,

BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013659, BRAMY2013722, BRAMY2013736, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015855, BRAMY2015890, BRAMY2015911, BRAMY2015925, BRAMY2016002, BRAMY2016009, BRAMY2016020, BRAMY2016070, BRAMY2016251, BRAMY2016327, BRAMY2016386, BRAMY2016706, BRAMY2016771,

BRAMY2016892, BRAMY2016938, BRAMY2016953, BRAMY2017014, BRAMY2017249, BRAMY2017348, BRAMY2017450, BRAMY2017455, BRAMY2017528, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018785, BRAMY2019055, BRAMY2019111, BRAMY2019277, BRAMY2019300, BRAMY2019509, BRAMY2019600, BRAMY2019710, BRAMY2019963, BRAMY2019985, BRAMY2019989, BRAMY2020050, BRAMY2020058, BRAMY2020106, BRAMY2020270, BRAMY2020355, BRAMY2020412, BRAMY2020445, BRAMY2020466, BRAMY2020520, BRAMY2020574, BRAMY2020634, BRAMY2021040, BRAMY2021063, BRAMY2021098, BRAMY2021139, BRAMY2021276, BRAMY2021310, BRAMY2021388, BRAMY2021498,

BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022168, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863, BRAMY2024004, BRAMY2024073, BRAMY2024312, BRAMY2024374, BRAMY2024390, BRAMY2024416, BRAMY2024449, BRAMY2024489,

BRAMY2024497, BRAMY2024514, BRAMY2024530, BRAMY2024535, BRAMY2024545, BRAMY2024576, BRAMY2024593, BRAMY2024711, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026713, BRAMY2026778, BRAMY2026824, BRAMY2026899, BRAMY2026902, BRAMY2026904, BRAMY2026976, BRAMY2027073, BRAMY2027114, BRAMY2027140, BRAMY2027242, BRAMY2027396, BRAMY2027451, BRAMY2027461, BRAMY2027467, BRAMY2027587, BRAMY2027714, BRAMY2027717, BRAMY2027739,

BRAMY2027752, BRAMY2027782, BRAMY2028072, BRAMY2028183, BRAMY2028188, BRAMY2028269, BRAMY2028282, BRAMY2028472, BRAMY2028491, BRAMY2028516, BRAMY2028565, BRAMY2028593, BRAMY2028682, BRAMY2028783, BRAMY2028856, BRAMY2028914, BRAMY2029204, BRAMY2029602, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2031516, BRAMY2032014, BRAMY2032231, BRAMY2032242, BRAMY2032290, BRAMY2032311, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332,

BRAMY2033506, BRAMY2033594, BRAMY2033895, BRAMY2033914, BRAMY2034072, BRAMY2034185, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035923, BRAMY2036076, BRAMY2036079, BRAMY2036254, BRAMY2036266, BRAMY2036387, BRAMY2036396, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629, BRAMY2037800, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2039287, BRAMY2039300, BRAMY2039314, BRAMY2039341, BRAMY2039630,

BRAMY2039695, BRAMY2039728, BRAMY2039872, BRAMY2040051, BRAMY2040159, BRAMY2040214, BRAMY2040478, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069, BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871,

BRAMY2046984, BRAMY2046989, BRAMY2047169, BRAMY2047280, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002508, BRAMY3002620, BRAMY3002803, BRAMY3002805, BRAMY3002886, BRAMY3002991, BRAMY3003026, BRAMY3003109, BRAMY3003310, BRAMY3003566, BRAMY3003723, BRAMY3004126, BRAMY3004224, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761,

BRAMY3007078, BRAMY3007206, BRAMY3007311, BRAMY3007350, BRAMY3007449, BRAMY3007471, BRAMY3007609, BRAMY3007723, BRAMY3007968, BRAMY3008088, BRAMY3008096, BRAMY3008335, BRAMY3008466, BRAMY3008505, BRAMY3008650, BRAMY3008937, BRAMY3009158, BRAMY3009207, BRAMY3009556, BRAMY3009782, BRAMY3009811, BRAMY3009904, BRAMY3010008, BRAMY3010264, BRAMY3010321, BRAMY3010411, BRAMY3010492, BRAMY3010603, BRAMY3010654, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000007, BRAWH1000045, BRAWH1000094,

BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000082, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000417, BRAWH2000443, BRAWH2000471, BRAWH2000476, BRAWH2000482, BRAWH2000488, BRAWH2000494, BRAWH2000500, BRAWH2000503, BRAWH2000522, BRAWH2000554, BRAWH2000588, BRAWH2000595, BRAWH2000633, BRAWH2000643, BRAWH2000651, BRAWH2000682, BRAWH2000752, BRAWH2000859, BRAWH2000866, BRAWH2000892,

BRAWH2000901, BRAWH2000926, BRAWH2000984, BRAWH2001019, BRAWH2001037, BRAWH2001052, BRAWH2001103, BRAWH2001129, BRAWH2001137, BRAWH2001141, BRAWH2001159, BRAWH2001186, BRAWH2001203, BRAWH2001238, BRAWH2001239, BRAWH2001255, BRAWH2001355, BRAWH2001378, BRAWH2001395, BRAWH2001412, BRAWH2001418, BRAWH2001424, BRAWH2001436, BRAWH2001438, BRAWH2001459, BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686, BRAWH2001701, BRAWH2001842, BRAWH2001862, BRAWH2001873, BRAWH2002047, BRAWH2002060, BRAWH2002062, BRAWH2002125, BRAWH2002191, BRAWH2002318, BRAWH2002333, BRAWH2002352, BRAWH2002383,

BRAWH2002549, BRAWH2002560, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003135, BRAWH2003240, BRAWH2003307, BRAWH2003355, BRAWH2003366, BRAWH2003662, BRAWH2003689, BRAWH2003693, BRAWH2003832, BRAWH2003948, BRAWH2003964, BRAWH2004044, BRAWH2004068, BRAWH2004078, BRAWH2004217, BRAWH2004580, BRAWH2004731, BRAWH2004779, BRAWH2004842, BRAWH2004884, BRAWH2004923, BRAWH2005008, BRAWH2005074, BRAWH2005225, BRAWH2005315, BRAWH2005517, BRAWH2005524, BRAWH2005533, BRAWH2005578, BRAWH2005812, BRAWH2005974, BRAWH2005998, BRAWH2006083, BRAWH2006207,

BRAWH2006301, BRAWH2006526, BRAWH2006543, BRAWH2006622, BRAWH2006760, BRAWH2006960, BRAWH2006989, BRAWH2007069, BRAWH2007308, BRAWH2007406, BRAWH2007550, BRAWH2007570, BRAWH2007591, BRAWH2007658, BRAWH2007664, BRAWH2007800, BRAWH2007808, BRAWH2007825, BRAWH2007862, BRAWH2007890, BRAWH2008058, BRAWH2008128, BRAWH2008255, BRAWH2008292, BRAWH2008297, BRAWH2008380, BRAWH2008540, BRAWH2008577, BRAWH2008790, BRAWH2008903, BRAWH2008956, BRAWH2008993, BRAWH2009112, BRAWH2009227, BRAWH2009261, BRAWH2009263, BRAWH2009298, BRAWH2009304, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069,

BRAWH2010084, BRAWH2010136, BRAWH2010318, BRAWH2010329, BRAWH2010354, BRAWH2010536, BRAWH2010552, BRAWH2010584, BRAWH2010618, BRAWH2010619, BRAWH2010754, BRAWH2011066, BRAWH2011079, BRAWH2011096, BRAWH2011155, BRAWH2011166, BRAWH2011204, BRAWH2011286, BRAWH2011294, BRAWH2011343, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871,

BRAWH2013914, BRAWH2013941, BRAWH2013955, BRAWH2014234, BRAWH2014379, BRAWH2014414, BRAWH2014473, BRAWH2014511, BRAWH2014662, BRAWH2014717, BRAWH2014729, BRAWH2014876, BRAWH2014934, BRAWH2014954, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016166, BRAWH2016209, BRAWH2016221, BRAWH2016223, BRAWH2016439, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017542,

BRAWH2017635, BRAWH2017913, BRAWH2018206, BRAWH2018267, BRAWH2018282, BRAWH2018317, BRAWH2018506, BRAWH2018514, BRAWH2018527, BRAWH2018601, BRAWH2018745, BRAWH2018875, BRAWH2019053, BRAWH2019055, BRAWH2019198, BRAWH3000078, BRAWH3000088, BRAWH3000100, BRAWH3000166, BRAWH3000314, BRAWH3000329, BRAWH3000410, BRAWH3000446, BRAWH3000491, BRAWH3000604, BRAWH3000884, BRAWH3001053, BRAWH3001319, BRAWH3001326, BRAWH3001475, BRAWH3001636, BRAWH3001638, BRAWH3001712, BRAWH3001760, BRAWH3001783, BRAWH3001833, BRAWH3001879, BRAWH3001918, BRAWH3002433, BRAWH3002467, BRAWH3002513, BRAWH3002574, BRAWH3002600, BRAWH3002819, BRAWH3003019, BRAWH3003136, BRAWH3003244, BRAWH3003343, BRAWH3003522, BRAWH3003598,

BRAWH3003727, BRAWH3003801, BRAWH3003975, BRAWH3004222, BRAWH3004335, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331,

BRAWH3011402, BRAWH3011577, BRAWH3011623, BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021580, BRAWH3021641, BRAWH3021643, BRAWH3021724, BRAWH3022347, BRAWH3022431,

BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3026349, BRAWH3026394, BRAWH3026529, BRAWH3027420, BRAWH3027440, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571,

BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2000620, BRCAN2000639,

BRCAN2000763, BRCAN2000832, BRCAN2000923, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007426, BRCAN2007525, BRCAN2007700, BRCAN2008494, BRCAN2008528, BRCAN2008701,

BRCAN2008894, BRCAN2009156, BRCAN2009168, BRCAN2009203, BRCAN2009432, BRCAN2010374, BRCAN2010376, BRCAN2010547, BRCAN2010581, BRCAN2010665, BRCAN2011254, BRCAN2011316, BRCAN2011602, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490, BRCAN2018566,

BRCAN2018667, BRCAN2018748, BRCAN2018935, BRCAN2019002, BRCAN2019387, BRCAN2019653, BRCAN2019773, BRCAN2019907, BRCAN2020331, BRCAN2020412, BRCAN2020467, BRCAN2020710, BRCAN2020880, BRCAN2021024, BRCAN2021028, BRCAN2021325, BRCAN2021452, BRCAN2021669, BRCAN2021718, BRCAN2022126, BRCAN2022191, BRCAN2022472, BRCAN2022474, BRCAN2023347, BRCAN2023724, BRCAN2024451, BRCAN2024563, BRCAN2024572, BRCAN2025009, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027513, BRCAN2027593, BRCAN2027970, BRCAN2028021, BRCAN2028036, BRCAN2028319, BRCAN2028338, BRCAN2028355, BRCAN2028378, BRCAN2028460,

BRCAN2028545, BRCAN2028634, BRCAN2028702, BRCOC1000087, BRCOC1000094, BRCOC2000004, BRCOC2000047, BRCOC2000152, BRCOC2000184, BRCOC2000186, BRCOC2000333, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005951, BRCOC2006164, BRCOC2006243, BRCOC2006270, BRCOC2006639, BRCOC2006942, BRCOC2007034, BRCOC2007593, BRCOC2007733, BRCOC2007769,

BRCOC2008064, BRCOC2008225, BRCOC2008642, BRCOC2008993, BRCOC2009052, BRCOC2009196, BRCOC2009380, BRCOC2009638, BRCOC2010115, BRCOC2010510, BRCOC2010730, BRCOC2010980, BRCOC2010994, BRCOC2011010, BRCOC2011398, BRCOC2011418, BRCOC2011506, BRCOC2011769, BRCOC2011996, BRCOC2012229, BRCOC2012386, BRCOC2012551, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896, BRCOC2019903, BRCOC2019910, BRCOC2019934,

BRCOC2020122, BRCOC2020142, BRHIP1000058, BRHIP1000072, BRHIP1000108, BRHIP1000129, BRHIP1000258, BRHIP2000021, BRHIP2000079, BRHIP2000087, BRHIP2000210, BRHIP2000239, BRHIP2000245, BRHIP2000312, BRHIP2000359, BRHIP2000445, BRHIP2000506, BRHIP2000532, BRHIP2000621, BRHIP2000691, BRHIP2000819, BRHIP2000826, BRHIP2000920, BRHIP2001099, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003062, BRHIP2003242, BRHIP2003272, BRHIP2003748, BRHIP2003786, BRHIP2003828, BRHIP2004312, BRHIP2004355, BRHIP2004359, BRHIP2004814,

BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007741, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010487, BRHIP2010571, BRHIP2010593, BRHIP2010610, BRHIP2010723, BRHIP2010744, BRHIP2011080, BRHIP2011120, BRHIP2011199, BRHIP2011508, BRHIP2011576, BRHIP2011616,

BRHIP2011783, BRHIP2011838, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013972, BRHIP2014063, BRHIP2014285, BRHIP2014291, BRHIP2014373, BRHIP2014386, BRHIP2014387, BRHIP2014954, BRHIP2015224, BRHIP2015245, BRHIP2015356, BRHIP2015360, BRHIP2016005, BRHIP2016125, BRHIP2016788, BRHIP2016968, BRHIP2016990, BRHIP2017315, BRHIP2017385, BRHIP2017404, BRHIP2017542, BRHIP2017553, BRHIP2017642, BRHIP2017714, BRHIP2017780, BRHIP2017945, BRHIP2018014, BRHIP2018369, BRHIP2018612,

BRHIP2018650, BRHIP2018719, BRHIP2018805, BRHIP2018998, BRHIP2019007, BRHIP2019047, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020799, BRHIP2020842, BRHIP2020859, BRHIP2020930, BRHIP2021102, BRHIP2021289, BRHIP2021495, BRHIP2021615, BRHIP2021744, BRHIP2021929, BRHIP2022006, BRHIP2022054, BRHIP2022221, BRHIP2022228, BRHIP2022298, BRHIP2022467, BRHIP2022708, BRHIP2022986, BRHIP2023071, BRHIP2023229, BRHIP2023272, BRHIP2023292, BRHIP2023309, BRHIP2023369, BRHIP2023438, BRHIP2023695, BRHIP2023735, BRHIP2023773, BRHIP2023860,

BRHIP2024046, BRHIP2024165, BRHIP2024201, BRHIP2024347, BRHIP2024398, BRHIP2024549, BRHIP2024789, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027017, BRHIP2027054, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000017, BRHIP3000060, BRHIP3000070, BRHIP3000081, BRHIP3000131, BRHIP3000240, BRHIP3000377, BRHIP3000457, BRHIP3000475, BRHIP3000488,

BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001878, BRHIP3001964, BRHIP3002114, BRHIP3002124, BRHIP3002141, BRHIP3002363, BRHIP3002691, BRHIP3002920, BRHIP3002931, BRHIP3003063, BRHIP3003126, BRHIP3003340, BRHIP3003395, BRHIP3003484, BRHIP3003644, BRHIP3003688, BRHIP3003795, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968, BRHIP3005137, BRHIP3005231, BRHIP3005307, BRHIP3005574, BRHIP3005673, BRHIP3005768, BRHIP3005801, BRHIP3005944, BRHIP3006279, BRHIP3006390, BRHIP3006449, BRHIP3006683, BRHIP3006717,

BRHIP3006786, BRHIP3006950, BRHIP3007172, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008405, BRHIP3008565, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3013078, BRHIP3013271, BRHIP3013698,

BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3018278, BRHIP3018784, BRHIP3019643, BRHIP3019824, BRHIP3019880, BRHIP3019956, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160,

BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000295, BRSSN2000312, BRSSN2000518, BRSSN2000561, BRSSN2000715, BRSSN2000832, BRSSN2001213, BRSSN2001275, BRSSN2001342, BRSSN2001426, BRSSN2001503, BRSSN2001579, BRSSN2001758, BRSSN2001810, BRSSN2002857, BRSSN2002859, BRSSN2003012, BRSSN2003086, BRSSN2003093, BRSSN2003820, BRSSN2003841, BRSSN2003937, BRSSN2004142, BRSSN2004303, BRSSN2004304, BRSSN2004496, BRSSN2004679, BRSSN2004686, BRSSN2004710, BRSSN2004719, BRSSN2005847, BRSSN2006133, BRSSN2006390,

BRSSN2006575, BRSSN2006611, BRSSN2006644, BRSSN2006892, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009627, BRSSN2009630, BRSSN2009702, BRSSN2010019, BRSSN2010110, BRSSN2010587, BRSSN2010596, BRSSN2010830, BRSSN2011262, BRSSN2011653, BRSSN2011738, BRSSN2011843, BRSSN2012081, BRSSN2012103, BRSSN2012157, BRSSN2012198, BRSSN2012254, BRSSN2012439, BRSSN2012622, BRSSN2012691, BRSSN2012838, BRSSN2013095, BRSSN2013544, BRSSN2013733, BRSSN2013753, BRSSN2013874, BRSSN2014112, BRSSN2014294, BRSSN2014424, BRSSN2014513,

BRSSN2014556, BRSSN2014573, BRSSN2014636, BRSSN2014685, BRSSN2015199, BRSSN2015369, BRSSN2015493, BRSSN2015554, BRSSN2015707, BRSSN2015773, BRSSN2015907, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000097, BRSTN2000058, BRSTN2000070, BRSTN2000220, BRSTN2000312, BRSTN2000536, BRSTN2000872, BRSTN2001067, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583,

BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010089, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451, BRSTN2013171, BRSTN2013354, BRSTN2013931, BRSTN2014490, BRSTN2014633, BRSTN2014751, BRSTN2014770, BRSTN2015015, BRSTN2015978, BRSTN2016066, BRSTN2016335, BRSTN2016892, BRSTN2016918, BRSTN2016992, BRSTN2017151, BRSTN2017237, BRSTN2017379, BRSTN2017761, BRSTN2017771,

BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639,

BRTHA2004642, BRTHA2004812, BRTHA2004821, BRTHA2004978, BRTHA2004992, BRTHA2005005, BRTHA2005013, BRTHA2005448, BRTHA2005579, BRTHA2005831, BRTHA2005864, BRTHA2005942, BRTHA2005956, BRTHA2006075, BRTHA2006146, BRTHA2006194, BRTHA2006232, BRTHA2006720, BRTHA2006735, BRTHA2006974, BRTHA2007060, BRTHA2007122, BRTHA2007422, BRTHA2007603, BRTHA2007665, BRTHA2007998, BRTHA2008316, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884,

BRTHA2010907, BRTHA2011062, BRTHA2011194, BRTHA2011321, BRTHA2011351, BRTHA2011500, BRTHA2011641, BRTHA2011691, BRTHA2012129, BRTHA2012183, BRTHA2012189, BRTHA2012351, BRTHA2012392, BRTHA2012466, BRTHA2012555, BRTHA2012562, BRTHA2012980, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663, BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543,

BRTHA2016552, BRTHA2016918, BRTHA2017178, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027229,

BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2036055, BRTHA2036295, BRTHA2036660, BRTHA2037247, BRTHA2038279, BRTHA2038345, BRTHA2038353, BRTHA2038396, BRTHA3000252, BRTHA3000273, BRTHA3000296, BRTHA3000297, BRTHA3000325, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003023, BRTHA3003225, BRTHA3003339, BRTHA3003343,

BRTHA3003417, BRTHA3003449, BRTHA3003474, BRTHA3003704, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007532, BRTHA3007662, BRTHA3007685, BRTHA3007769, BRTHA3007922, BRTHA3008143, BRTHA3008293, BRTHA3008310, BRTHA3008386, BRTHA3008520, BRTHA3008608, BRTHA3008694, BRTHA3008826, BRTHA3009037, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011229, BRTHA3011265, BRTHA3011510, BRTHA3011892, BRTHA3012265, BRTHA3013860, BRTHA3013882,

BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048, BRTHA3019105, BRTHA3019183, BRTHA3020000, BRTHA3020369, BRTHA3021569, BRTHA3021708, BRTHA3021786, BRTHA3021971, BRTHA3022641, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026556, BRTHA3026916, BRTHA3027171, BRTHA3027318, BRTHA3027638,

BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000314, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382, CHONS2001797, CHONS2001834, CHONS2002419, COLON1000030, COLON1000084, COLON1000135, COLON2000470, COLON2000557, COLON2000568, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282,

COLON2006417, COLON2007274, COLON2007733, COLON2008105, COLON2009337, COLON2009499, CORDB1000140, CORDB2000061, CORDB2000127, CORDB2000541, CTONG1000009, CTONG1000010, CTONG1000020, CTONG1000022, CTONG1000040, CTONG1000056, CTONG1000062, CTONG1000086, CTONG1000088, CTONG1000094, CTONG1000097, CTONG1000113, CTONG1000137, CTONG1000165, CTONG1000175, CTONG1000180, CTONG1000181, CTONG1000220, CTONG1000277, CTONG1000283, CTONG1000288, CTONG1000302, CTONG1000341, CTONG1000467, CTONG1000476, CTONG1000488, CTONG1000508, CTONG1000540, CTONG2000031, CTONG2000034, CTONG2000042, CTONG2000218, CTONG2000279, CTONG2000303, CTONG2000330, CTONG2000395, CTONG2000411, CTONG2000465, CTONG2000480, CTONG2000561,

CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001413, CTONG2001428, CTONG2001513, CTONG2001524, CTONG2001533, CTONG2001598, CTONG2001681, CTONG2001724, CTONG2001749, CTONG2001820, CTONG2001877, CTONG2001911, CTONG2001932, CTONG2001955, CTONG2002066, CTONG2002395, CTONG2002417, CTONG2002418, CTONG2002453, CTONG2002520, CTONG2002558, CTONG2002590, CTONG2002709, CTONG2002721, CTONG2002816, CTONG2002820, CTONG2002903, CTONG2002965, CTONG2002999, CTONG2003028, CTONG2003094, CTONG2003100, CTONG2003115, CTONG2003245, CTONG2003348, CTONG2003350, CTONG2003372, CTONG2003375, CTONG2003427,

CTONG2003599, CTONG2003680, CTONG2003699, CTONG2003764, CTONG2003782, CTONG2003889, CTONG2003937, CTONG2004000, CTONG2004062, CTONG2004115, CTONG2004126, CTONG2004264, CTONG2004305, CTONG2004397, CTONG2004423, CTONG2004454, CTONG2004550, CTONG2004669, CTONG2004716, CTONG2004912, CTONG2004941, CTONG2004948, CTONG2005028, CTONG2005049, CTONG2005110, CTONG2005145, CTONG2005265, CTONG2005290, CTONG2005374, CTONG2005399, CTONG2005468, CTONG2005567, CTONG2005585, CTONG2005635, CTONG2005775, CTONG2005795, CTONG2005890, CTONG2005904, CTONG2006129, CTONG2006223, CTONG2006273, CTONG2006310, CTONG2006393, CTONG2006449, CTONG2006515, CTONG2006524, CTONG2006568, CTONG2006611, CTONG2006620, CTONG2006666,

CTONG2006691, CTONG2006709, CTONG2006836, CTONG2006942, CTONG2006964, CTONG2007009, CTONG2007091, CTONG2007168, CTONG2007175, CTONG2007259, CTONG2007272, CTONG2007293, CTONG2007297, CTONG2007400, CTONG2007417, CTONG2007441, CTONG2007623, CTONG2007681, CTONG2007779, CTONG2007898, CTONG2007959, CTONG2007970, CTONG2008014, CTONG2008184, CTONG2008262, CTONG2008269, CTONG2008321, CTONG2008343, CTONG2008402, CTONG2008521, CTONG2008562, CTONG2008577, CTONG2008595, CTONG2008621, CTONG2008689, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395,

CTONG2009423, CTONG2009440, CTONG2009527, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009923, CTONG2009938, CTONG2009955, CTONG2009958, CTONG2010018, CTONG2010024, CTONG2010116, CTONG2010133, CTONG2010148, CTONG2010276, CTONG2010330, CTONG2010348, CTONG2010408, CTONG2010508, CTONG2010566, CTONG2010623, CTONG2010649, CTONG2010803, CTONG2011188, CTONG2011429, CTONG2011474, CTONG2011676, CTONG2011716, CTONG2011765, CTONG2011779, CTONG2011815, CTONG2011822, CTONG2011825, CTONG2011920, CTONG2011985, CTONG2012029, CTONG2012077,

CTONG2012123, CTONG2012158, CTONG2012230, CTONG2012401, CTONG2012422, CTONG2012425, CTONG2012447, CTONG2012453, CTONG2012473, CTONG2012476, CTONG2012533, CTONG2012554, CTONG2012564, CTONG2012661, CTONG2012724, CTONG2012738, CTONG2012758, CTONG2012843, CTONG2012879, CTONG2012901, CTONG2012996, CTONG2013128, CTONG2013149, CTONG2013156, CTONG2013284, CTONG2013339, CTONG2013352, CTONG2013381, CTONG2013393, CTONG2013511, CTONG2013630, CTONG2013803, CTONG2013907, CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014389, CTONG2014491, CTONG2014630, CTONG2014635, CTONG2014646, CTONG2014705,

CTONG2014946, CTONG2014954, CTONG2014959, CTONG2014966, CTONG2014995, CTONG2015204, CTONG2015316, CTONG2015345, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016056, CTONG2016217, CTONG2016355, CTONG2016499, CTONG2016505, CTONG2016559, CTONG2016658, CTONG2016775, CTONG2016797, CTONG2016824, CTONG2016846, CTONG2016904, CTONG2016953, CTONG2017021, CTONG2017292, CTONG2017409, CTONG2017444, CTONG2017458, CTONG2017604, CTONG2017650, CTONG2017804, CTONG2017814, CTONG2017910, CTONG2017939, CTONG2017989, CTONG2018062, CTONG2018069, CTONG2018147, CTONG2018211, CTONG2018217,

CTONG2018279, CTONG2018334, CTONG2018343, CTONG2018379, CTONG2018413, CTONG2018483, CTONG2018629, CTONG2018653, CTONG2018655, CTONG2018735, CTONG2018900, CTONG2018976, CTONG2019029, CTONG2019063, CTONG2019144, CTONG2019232, CTONG2019248, CTONG2019590, CTONG2019652, CTONG2019704, CTONG2019822, CTONG2019833, CTONG2020031, CTONG2020108, CTONG2020127, CTONG2020171, CTONG2020374, CTONG2020378, CTONG2020411, CTONG2020445, CTONG2020806, CTONG2020831, CTONG2021024, CTONG2021132, CTONG2021168, CTONG2022153, CTONG2022601, CTONG2022835, CTONG2023021, CTONG2023512, CTONG2024031, CTONG2024206, CTONG2024614, CTONG2024749, CTONG2025496, CTONG2025513, CTONG2025516, CTONG2025610, CTONG2025900, CTONG2026513,

CTONG2026770, CTONG2026920, CTONG2026987, CTONG2027150, CTONG2027327, CTONG2027361, CTONG2027591, CTONG2027736, CTONG2027783, CTONG2027828, CTONG2027959, CTONG2028097, CTONG2028208, CTONG2028486, CTONG2028518, CTONG2028687, CTONG3000657, CTONG3000686, CTONG3000707, CTONG3000896, CTONG3001420, CTONG3001501, CTONG3001560, CTONG3001605, CTONG3001746, CTONG3002020, CTONG3002127, CTONG3002370, CTONG3002412, CTONG3002433, CTONG3002552, CTONG3002588, CTONG3002674, CTONG3002728, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004317,

CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009227, CTONG3009287, CTONG3009328, CTONG3009385, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001788, D3OST2002182, D3OST2002223, D3OST2002262,

D3OST2002272, D3OST2002417, D3OST2002436, D3OST2002648, D3OST2003331, D3OST2003607, D3OST2003797, D3OST2004637, D3OST3000195, D3OST3000258, D3OST3000291, D3OST3000388, D3OST3000493, D6OST2000127, D6OST2000169, D6OST2000245, D6OST2000358, D6OST2000464, D6OST2000507, D9OST2000278, D9OST2000440, D9OST2000869, D9OST2001319, D9OST2001433, D9OST2002397, D9OST2002608, D9OST2002673, D9OST2003106, D9OST2003108, D9OST2003137, D9OST2003397, D9OST2003580, D9OST2003594, D9OST2003791, D9OST2003989, D9OST2004018, D9OST2004073, D9OST2004417, DFNES1000003, DFNES1000083, DFNES1000107, DFNES1000185, DFNES2000143, DFNES2000146, DFNES2000212, DFNES2000292, DFNES2000426, DFNES2000432, DFNES2000457, DFNES2000471,

DFNES2000913, DFNES2001091, DFNES2001096, DFNES2001108, DFNES2001177, DFNES2001404, DFNES2001564, DFNES2001565, DFNES2001647, DFNES2001800, DFNES2001829, DFNES2002220, DFNES2002509, DFNES2002550, DFNES2002588, DFNES2002817, DFNES2003081, DFNES2003255, DFNES2003742, DFNES2004354, DFNES2004383, DFNES2004608, DFNES2004684, DFNES2005266, DFNES2005527, DFNES2005540, DFNES2005690, DFNES2005766, DFNES2005779, DFNES2005875, DFNES2006346, DFNES2006944, DFNES2007299, DFNES2007332, DFNES2007641, DFNES2007757, DFNES2008160, DFNES2008280, DFNES2008881, DFNES2009482, DFNES2010502, DFNES2011192, DFNES2011221, DFNES2011239, DFNES2011245, ERLTF1000021, ERLTF2000324, ERLTF2000465, FCBBF1000023, FCBBF1000025,

FCBBF1000038, FCBBF1000053, FCBBF1000069, FCBBF1000115, FCBBF1000117, FCBBF1000118, FCBBF1000121, FCBBF1000125, FCBBF1000131, FCBBF1000155, FCBBF1000157, FCBBF1000171, FCBBF1000174, FCBBF1000182, FCBBF1000198, FCBBF1000206, FCBBF1000220, FCBBF1000232, FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000367, FCBBF1000376, FCBBF1000377, FCBBF1000395, FCBBF1000397, FCBBF1000403, FCBBF1000412, FCBBF1000437, FCBBF1000449, FCBBF1000466, FCBBF1000476, FCBBF1000506, FCBBF1000509, FCBBF1000546, FCBBF1000550, FCBBF1000556, FCBBF1000581, FCBBF1000582, FCBBF1000598, FCBBF1000604, FCBBF1000618, FCBBF1000627,

FCBBF1000671, FCBBF1000675, FCBBF1000684, FCBBF1000686, FCBBF1000687, FCBBF1000691, FCBBF1000732, FCBBF1000748, FCBBF2000038, FCBBF2000094, FCBBF2000105, FCBBF2000123, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566, FCBBF2000677, FCBBF2000678, FCBBF2000685, FCBBF2000733, FCBBF2000782, FCBBF2000808, FCBBF2000815, FCBBF2000825, FCBBF2000940, FCBBF2000951, FCBBF2001001, FCBBF2001088, FCBBF2001105, FCBBF2001116, FCBBF2001183, FCBBF2001188, FCBBF2001211, FCBBF2001238, FCBBF2001243, FCBBF2001291, FCBBF2001299, FCBBF2001309, FCBBF2001400, FCBBF2001427, FCBBF2001480, FCBBF2001529, FCBBF2001538, FCBBF2001594,

FCBBF2001672, FCBBF2001675, FCBBF2001718, FCBBF2001720, FCBBF2001817, FCBBF2001868, FCBBF2002044, FCBBF2002281, FCBBF2002370, FCBBF2002541, FCBBF2002626, FCBBF2002897, FCBBF2002898, FCBBF2002904, FCBBF2002928, FCBBF2002953, FCBBF2003083, FCBBF2003269, FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005733, FCBBF2005909, FCBBF2005966, FCBBF2006131, FCBBF2006380,

FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000115, FCBBF3000120, FCBBF3000168, FCBBF3000175, FCBBF3000227, FCBBF3000229, FCBBF3000233, FCBBF3000269, FCBBF3000361, FCBBF3000367, FCBBF3000462, FCBBF3000473, FCBBF3000501, FCBBF3000518, FCBBF3000550, FCBBF3000847, FCBBF3001018, FCBBF3001020, FCBBF3001235, FCBBF3001302, FCBBF3001594, FCBBF3001657, FCBBF3001855, FCBBF3001890, FCBBF3001912, FCBBF3001914, FCBBF3001918, FCBBF3001924, FCBBF3002163, FCBBF3002188, FCBBF3002190, FCBBF3002268, FCBBF3002475, FCBBF3002556, FCBBF3002592, FCBBF3002658, FCBBF3002782,

FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004323, FCBBF3004390, FCBBF3004409, FCBBF3004502, FCBBF3004715, FCBBF3004842, FCBBF3004847, FCBBF3004955, FCBBF3005160, FCBBF3005218, FCBBF3005320, FCBBF3005444, FCBBF3005698, FCBBF3005729, FCBBF3006171, FCBBF3006249, FCBBF3006288, FCBBF3006399, FCBBF3006628, FCBBF3007077, FCBBF3007150, FCBBF3007152, FCBBF3007242, FCBBF3007244, FCBBF3007248, FCBBF3007453, FCBBF3007462, FCBBF3007540, FCBBF3007604, FCBBF3007631, FCBBF3007829, FCBBF3007855, FCBBF3007859, FCBBF3007860, FCBBF3008073, FCBBF3008100, FCBBF3008153,

FCBBF3008251, FCBBF3008311, FCBBF3008362, FCBBF3008382, FCBBF3008475, FCBBF3008644, FCBBF3008689, FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010695, FCBBF3010729, FCBBF3010733, FCBBF3011003, FCBBF3011418, FCBBF3011523, FCBBF3011570, FCBBF3011592, FCBBF3011867, FCBBF3011889, FCBBF3012170, FCBBF3012182,

FCBBF3012332, FCBBF3012443, FCBBF3012588, FCBBF3012667, FCBBF3012842, FCBBF3012901, FCBBF3013041, FCBBF3013058, FCBBF3013205, FCBBF3013307, FCBBF3013341, FCBBF3013589, FCBBF3013623, FCBBF3013800, FCBBF3013846, FCBBF3014229, FCBBF3014260, FCBBF3014355, FCBBF3014567, FCBBF3015100, FCBBF3015119, FCBBF3015131, FCBBF3015317, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016987, FCBBF3017024, FCBBF3017059, FCBBF3017071, FCBBF3017123, FCBBF3017233, FCBBF3017255, FCBBF3017288, FCBBF3017535, FCBBF3017681, FCBBF3017729, FCBBF3017873, FCBBF3017974, FCBBF3018067, FCBBF3018173, FCBBF3018453,

FCBBF3018591, FCBBF3018753, FCBBF3018826, FCBBF3018949, FCBBF3019085, FCBBF3019320, FCBBF3019437, FCBBF3019564, FCBBF3019569, FCBBF3019570, FCBBF3019663, FCBBF3019714, FCBBF3019716, FCBBF3019784, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078, FCBBF3023368, FCBBF3023372, FCBBF3023443, FCBBF3023599, FCBBF3023704, FCBBF3023887, FCBBF3023895, FCBBF3024096, FCBBF3024225, FCBBF3024266,

FCBBF3024364, FCBBF3024623, FCBBF3024793, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025905, FCBBF3026021, FCBBF3026048, FCBBF3026251, FCBBF3026308, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028244, FCBBF3028472, FCBBF3028528, FCBBF3028582, FCBBF3028671, FCBBF3028794, FCBBF4000017, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415,

FCBBF4000529, FCBBF5000041, FCBBF5000061, FCBBF5000165, FCBBF5000325, FCBBF5000353, FCBBF5000384, FCBBF5000483, FCBBF5000495, FEBRA1000005, FEBRA1000008, FEBRA1000030, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000129, FEBRA2000220, FEBRA2000228, FEBRA2000253, FEBRA2000286, FEBRA2000297, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000454, FEBRA2000462, FEBRA2000468, FEBRA2000491, FEBRA2000504, FEBRA2000536, FEBRA2000538,

FEBRA2000575, FEBRA2000581, FEBRA2000656, FEBRA2000659, FEBRA2000680, FEBRA2000690, FEBRA2000727, FEBRA2000734, FEBRA2000757, FEBRA2000771, FEBRA2000772, FEBRA2000782, FEBRA2000787, FEBRA2000790, FEBRA2000793, FEBRA2000803, FEBRA2000805, FEBRA2000809, FEBRA2000815, FEBRA2000862, FEBRA2000877, FEBRA2000880, FEBRA2000901, FEBRA2000909, FEBRA2000917, FEBRA2000928, FEBRA2000936, FEBRA2000937, FEBRA2000959, FEBRA2001004, FEBRA2001012, FEBRA2001020, FEBRA2001093, FEBRA2001124, FEBRA2001146, FEBRA2001161, FEBRA2001175, FEBRA2001197, FEBRA2001217, FEBRA2001227, FEBRA2001245, FEBRA2001267, FEBRA2001294, FEBRA2001351, FEBRA2001383, FEBRA2001438, FEBRA2001440, FEBRA2001487, FEBRA2001561, FEBRA2001581,

FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001745, FEBRA2001772, FEBRA2001790, FEBRA2001814, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002442, FEBRA2002456, FEBRA2002508, FEBRA2002525, FEBRA2002552, FEBRA2002574, FEBRA2002601, FEBRA2002611, FEBRA2002628, FEBRA2002662, FEBRA2002682, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931,

FEBRA2002962, FEBRA2002986, FEBRA2003054, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003436, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003750, FEBRA2003822, FEBRA2003897, FEBRA2003907, FEBRA2003930, FEBRA2004026, FEBRA2004029, FEBRA2004042, FEBRA2004053, FEBRA2004056, FEBRA2004091, FEBRA2004110, FEBRA2004202, FEBRA2004219, FEBRA2004237, FEBRA2004268, FEBRA2004325, FEBRA2004329, FEBRA2004331, FEBRA2004443, FEBRA2004485, FEBRA2004490, FEBRA2004538, FEBRA2004592,

FEBRA2004620, FEBRA2004628, FEBRA2004651, FEBRA2004767, FEBRA2004813, FEBRA2004818, FEBRA2004840, FEBRA2004852, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005427, FEBRA2005458, FEBRA2005476, FEBRA2005572, FEBRA2005701, FEBRA2005726, FEBRA2005778, FEBRA2005787, FEBRA2005788, FEBRA2005824, FEBRA2005998, FEBRA2006055, FEBRA2006061, FEBRA2006150, FEBRA2006165, FEBRA2006255, FEBRA2006315, FEBRA2006346, FEBRA2006354, FEBRA2006357, FEBRA2006370, FEBRA2006379, FEBRA2006396, FEBRA2006409, FEBRA2006418, FEBRA2006427, FEBRA2006476, FEBRA2006485, FEBRA2006491,

FEBRA2006519, FEBRA2006521, FEBRA2006553, FEBRA2006627, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017, FEBRA2007176, FEBRA2007247, FEBRA2007280, FEBRA2007414, FEBRA2007436, FEBRA2007449, FEBRA2007458, FEBRA2007534, FEBRA2007544, FEBRA2007551, FEBRA2007566, FEBRA2007589, FEBRA2007613, FEBRA2007714, FEBRA2007793, FEBRA2007818, FEBRA2007823, FEBRA2007880, FEBRA2007931, FEBRA2007957, FEBRA2008009, FEBRA2008086, FEBRA2008134, FEBRA2008135, FEBRA2008159, FEBRA2008165, FEBRA2008172, FEBRA2008201, FEBRA2008210, FEBRA2008255, FEBRA2008265, FEBRA2008266, FEBRA2008282, FEBRA2008287, FEBRA2008302,

FEBRA2008311, FEBRA2008341, FEBRA2008360, FEBRA2008459, FEBRA2008468, FEBRA2008475, FEBRA2008481, FEBRA2008542, FEBRA2008660, FEBRA2008662, FEBRA2008681, FEBRA2008692, FEBRA2008741, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009352, FEBRA2009362, FEBRA2009385, FEBRA2009416, FEBRA2009437, FEBRA2009478, FEBRA2009507, FEBRA2009541, FEBRA2009567, FEBRA2009588, FEBRA2009731, FEBRA2009754, FEBRA2009780, FEBRA2009846, FEBRA2010141, FEBRA2010299, FEBRA2010484, FEBRA2010719,

FEBRA2010746, FEBRA2010768, FEBRA2010802, FEBRA2011063, FEBRA2011090, FEBRA2011356, FEBRA2011392, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013699, FEBRA2013905, FEBRA2013951, FEBRA2014010, FEBRA2014122, FEBRA2014198, FEBRA2014213, FEBRA2014443, FEBRA2014578, FEBRA2014939, FEBRA2015076, FEBRA2015175, FEBRA2015292, FEBRA2015588, FEBRA2016112, FEBRA2016398, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333,

FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2019172, FEBRA2019242, FEBRA2019298, FEBRA2019389, FEBRA2019457, FEBRA2019519, FEBRA2019582, FEBRA2019637, FEBRA2019690, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021431, FEBRA2021497, FEBRA2021550, FEBRA2021571, FEBRA2021636, FEBRA2021864, FEBRA2021892, FEBRA2021908, FEBRA2022148, FEBRA2022204, FEBRA2022255, FEBRA2022322, FEBRA2022601, FEBRA2022696, FEBRA2022729, FEBRA2022787, FEBRA2022911, FEBRA2022956,

FEBRA2022963, FEBRA2022972, FEBRA2023227, FEBRA2023285, FEBRA2023351, FEBRA2023377, FEBRA2023399, FEBRA2023498, FEBRA2023550, FEBRA2023721, FEBRA2023927, FEBRA2023989, FEBRA2023990, FEBRA2024019, FEBRA2024136, FEBRA2024343, FEBRA2024570, FEBRA2024693, FEBRA2024727, FEBRA2024744, FEBRA2024797, FEBRA2025249, FEBRA2025463, FEBRA2025477, FEBRA2026474, FEBRA2026582, FEBRA2026629, FEBRA2026769, FEBRA2026879, FEBRA2026890, FEBRA2026977, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEHRT2000325, FEHRT2000364, FEKID1000018, FELIV1000101, FELIV1000137, FELIV1000164, FELNG2000241, HCASM1000021,

HCASM1000050, HCASM1000061, HCASM1000130, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003076, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405, HCASM2009424, HCASM2009463, HCASM2009588, HCHON1000015, HCHON1000142, HCHON1000176, HCHON1000191, HCHON2000028, HCHON2000038, HCHON2000062, HCHON2000100,

HCHON2000156, HCHON2000199, HCHON2000212, HCHON2000271, HCHON2000295, HCHON2000315, HCHON2000322, HCHON2000344, HCHON2000364, HCHON2000395, HCHON2000417, HCHON2000418, HCHON2000473, HCHON2000503, HCHON2000660, HCHON2000673, HCHON2000676, HCHON2000699, HCHON2000705, HCHON2000743, HCHON2000751, HCHON2000817, HCHON2000818, HCHON2000819, HCHON2000832, HCHON2000851, HCHON2000898, HCHON2000935, HCHON2000942, HCHON2000956, HCHON2000994, HCHON2001050, HCHON2001084, HCHON2001108, HCHON2001110, HCHON2001116, HCHON2001161, HCHON2001214, HCHON2001233, HCHON2001269, HCHON2001434, HCHON2001450, HCHON2001453, HCHON2001497, HCHON2001523, HCHON2001546, HCHON2001548, HCHON2001577, HCHON2001604, HCHON2001607,

HCHON2001646, HCHON2001712, HCHON2001768, HCHON2001801, HCHON2001853, HCHON2002123, HCHON2002194, HCHON2002241, HCHON2002247, HCHON2002354, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005987, HCHON2006459, HCHON2006714, HCHON2006722, HCHON2006786, HCHON2006841, HCHON2006871, HCHON2007046, HCHON2007482, HCHON2007650, HCHON2007674, HCHON2007774, HCHON2007881, HCHON2008112, HCHON2008239, HCHON2008444, HCHON2008582, HCHON2008672,

HCHON2008856, HCHON2008871, HCHON2008989, HCHON2009191, HCHON2009384, HCHON2009749, HCHON2009766, HCHON2009795, HCHON2010074, HCHON2010543, HEART1000074, HEART1000088, HEART1000099, HEART1000102, HEART1000118, HEART1000132, HEART1000139, HEART1000142, HEART1000151, HEART1000173, HEART2000024, HEART2000035, HEART2000099, HEART2000298, HEART2000306, HEART2000309, HEART2000411, HEART2000418, HEART2000448, HEART2000492, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003852,

HEART2004570, HEART2004940, HEART2004980, HEART2005244, HEART2005581, HEART2005610, HEART2006131, HEART2006195, HEART2006310, HEART2006334, HEART2006487, HEART2006557, HEART2006787, HEART2006789, HEART2006909, HEART2007031, HEART2007231, HEART2007443, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC2000055, HHDPC2000149, HHDPC2000258, HHDPC2000315, HHDPC2000456, HHDPC2000462, HHDPC2000572, HHDPC2000656, HHDPC2000692, HHDPC2001337, HHDPC2001432, HHDPC2001896, HHDPC2002963, HHDPC2003049, HHDPC2003113, HHDPC2003381,

HHDPC2004757, HHDPC2005185, HHDPC2005513, HHDPC2005742, HHDPC2006460, HHDPC2006862, HHDPC2006927, HHDPC2007302, HHDPC2007775, HHDPC2008123, HHDPC2008185, HHDPC2008279, HHDPC2008297, HHDPC2008414, HHDPC2008474, HHDPC2008816, HHDPC2008843, HHDPC2009114, HHDPC2009208, HHDPC2009272, HLUNG1000017, HLUNG1000024, HLUNG1000030, HLUNG1000034, HLUNG1000053, HLUNG1000064, HLUNG1000076, HLUNG1000084, HLUNG1000091, HLUNG1000104, HLUNG1000105, HLUNG1000151, HLUNG1000169, HLUNG2000014, HLUNG2000027, HLUNG2000063, HLUNG2000068, HLUNG2000116, HLUNG2000125, HLUNG2000130, HLUNG2000142, HLUNG2000174, HLUNG2000176, HLUNG2000217, HLUNG2000232, HLUNG2000255, HLUNG2000281, HLUNG2000315, HLUNG2000340, HLUNG2000362,

HLUNG2000363, HLUNG2000412, HLUNG2000431, HLUNG2000480, HLUNG2000489, HLUNG2000549, HLUNG2000557, HLUNG2000570, HLUNG2000751, HLUNG2000761, HLUNG2000777, HLUNG2000870, HLUNG2000926, HLUNG2000950, HLUNG2000955, HLUNG2000970, HLUNG2001013, HLUNG2001126, HLUNG2001144, HLUNG2001214, HLUNG2001439, HLUNG2001459, HLUNG2001507, HLUNG2001633, HLUNG2001677, HLUNG2001712, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002465, HLUNG2002562, HLUNG2002659, HLUNG2002707, HLUNG2002811, HLUNG2002916, HLUNG2002942, HLUNG2002949, HLUNG2002958, HLUNG2002982, HLUNG2003049, HLUNG2003061,

HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004037, HLUNG2004067, HLUNG2004154, HLUNG2004170, HLUNG2004217, HLUNG2004273, HLUNG2004388, HLUNG2004416, HLUNG2004521, HLUNG2004684, HLUNG2004707, HLUNG2004774, HLUNG2004775, HLUNG2005012, HLUNG2005030, HLUNG2005045, HLUNG2005076, HLUNG2005203, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006370, HLUNG2006397, HLUNG2006570, HLUNG2006599, HLUNG2006614,

HLUNG2006671, HLUNG2006781, HLUNG2006812, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008153, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008833, HLUNG2008875, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049, HLUNG2012287, HLUNG2012315, HLUNG2012546, HLUNG2012600, HLUNG2012727, HLUNG2013097,

HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015184, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282, HLUNG2018626, HLUNG2019058, HSYRA1000131, HSYRA2000044, HSYRA2000135, HSYRA2000137, HSYRA2000221, HSYRA2000237, HSYRA2000248, HSYRA2000253, HSYRA2000332, HSYRA2000371, HSYRA2000424, HSYRA2000510, HSYRA2000605, HSYRA2000629, HSYRA2000706, HSYRA2000760, HSYRA2000792, HSYRA2000828, HSYRA2000832, HSYRA2000925, HSYRA2000927, HSYRA2001003, HSYRA2001105,

HSYRA2001142, HSYRA2001153, HSYRA2001232, HSYRA2001255, HSYRA2001332, HSYRA2001353, HSYRA2001484, HSYRA2001552, HSYRA2001574, HSYRA2001580, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000158, IMR321000161, IMR321000165, IMR321000193, IMR321000207, IMR321000230, IMR322000072, IMR322000107, IMR322000121, IMR322000127, IMR322000152, IMR322000164, IMR322000178, IMR322000302, IMR322000316, IMR322000374, IMR322000379, IMR322000383, IMR322000418,

IMR322000518, IMR322000604, IMR322000609, IMR322000656, IMR322000672, IMR322000775, IMR322000791, IMR322000811, IMR322000819, IMR322000838, IMR322000859, IMR322000863, IMR322000917, IMR322000918, IMR322000919, IMR322000935, IMR322000953, IMR322000973, IMR322000984, IMR322001018, IMR322001049, IMR322001167, IMR322001185, IMR322001218, IMR322001245, IMR322001317, IMR322001318, IMR322001322, IMR322001438, IMR322001452, IMR322001534, IMR322001541, IMR322001665, IMR322001670, IMR322001724, IMR322001879, IMR322002035, IMR322002110, IMR322002470, IMR322003675, IMR322004768, IMR322005293, IMR322005833, IMR322006012, IMR322006222, IMR322006495, IMR322006520, IMR322006886, IMR322006947, IMR322007078,

IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, JCMLC2000273, KIDNE1000008, KIDNE1000012, KIDNE1000015, KIDNE1000028, KIDNE1000036, KIDNE1000064, KIDNE1000104, KIDNE1000121, KIDNE1000143, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000287, KIDNE2000315, KIDNE2000317, KIDNE2000328, KIDNE2000330, KIDNE2000335, KIDNE2000349, KIDNE2000375, KIDNE2000383,

KIDNE2000394, KIDNE2000422, KIDNE2000493, KIDNE2000510, KIDNE2000513, KIDNE2000519, KIDNE2000555, KIDNE2000624, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2000909, KIDNE2001117, KIDNE2001140, KIDNE2001269, KIDNE2001361, KIDNE2001373, KIDNE2001636, KIDNE2001713, KIDNE2001802, KIDNE2001840, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002262, KIDNE2002265, KIDNE2002438, KIDNE2002483, KIDNE2002725, KIDNE2002768, KIDNE2002795, KIDNE2002798, KIDNE2002839, KIDNE2002845, KIDNE2002872, KIDNE2002882,

KIDNE2002980, KIDNE2002991, KIDNE2003185, KIDNE2003188, KIDNE2003233, KIDNE2003335, KIDNE2003373, KIDNE2003377, KIDNE2003507, KIDNE2003752, KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004305, KIDNE2004344, KIDNE2004394, KIDNE2004411, KIDNE2004475, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400,

KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376, KIDNE2006378, KIDNE2006465, KIDNE2006580, KIDNE2006652, KIDNE2006687, KIDNE2006733, KIDNE2006811, KIDNE2006820, KIDNE2006880, KIDNE2007005, KIDNE2007040, KIDNE2007077, KIDNE2007186, KIDNE2007222, KIDNE2007356, KIDNE2007422, KIDNE2007569, KIDNE2007810, KIDNE2007811, KIDNE2007944, KIDNE2008069, KIDNE2008072, KIDNE2008116, KIDNE2008117, KIDNE2008218, KIDNE2008315, KIDNE2008362, KIDNE2008378,

KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191, KIDNE2009332, KIDNE2009426, KIDNE2009467, KIDNE2009490, KIDNE2009553, KIDNE2009605, KIDNE2009628, KIDNE2009647, KIDNE2009676, KIDNE2009723, KIDNE2010007, KIDNE2010049, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010762, KIDNE2010863, KIDNE2010973, KIDNE2010989, KIDNE2011200,

KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012440, KIDNE2012453, KIDNE2012563, KIDNE2012601, KIDNE2012613, KIDNE2012710, KIDNE2012745, KIDNE2012775, KIDNE2012784, KIDNE2012945, KIDNE2013045, KIDNE2013095, KIDNE2013158, KIDNE2013218, KIDNE2013263, KIDNE2013388, KIDNE2013413, KIDNE2013489, KIDNE2013639, KIDNE2013731, KIDNE2013734, KIDNE2013775, KIDNE2013801, KIDNE2014087, KIDNE2014112, KIDNE2014119, KIDNE2014170, KIDNE2014184, KIDNE2014268, KIDNE2014298, KIDNE2014320, KIDNE2014325, KIDNE2014415, KIDNE2014489, KIDNE2014496, KIDNE2014661, KIDNE2014717,

KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015987, KIDNE2016000, KIDNE2016036, KIDNE2016096, KIDNE2016101, KIDNE2016142, KIDNE2016188, KIDNE2016327, KIDNE2016371, KIDNE2016388, KIDNE2016464, KIDNE2016539, KIDNE2016918, KIDNE2017007, KIDNE2017040, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018254, KIDNE2018268, KIDNE2018269, KIDNE2018287, KIDNE2018294, KIDNE2018462, KIDNE2018493,

KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000082, LIVER1000099, LIVER1000104, LIVER1000111, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER2000003, LIVER2000037, LIVER2000158, LIVER2000216, LIVER2000237, LIVER2000247, LIVER2000360, LIVER2000515, LIVER2000592, LIVER2000626, LIVER2000682, LIVER2000769, LIVER2000775, LIVER2001051, LIVER2001076, LIVER2001099, LIVER2001164,

LIVER2001191, LIVER2001265, LIVER2001329, LIVER2001389, LIVER2001461, LIVER2001835, LIVER2002638, LIVER2002644, LIVER2002660, LIVER2002702, LIVER2003008, LIVER2003065, LIVER2003234, LIVER2003511, LIVER2003524, LIVER2003568, LIVER2003800, LIVER2003854, LIVER2004074, LIVER2004392, LIVER2004514, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005973, LIVER2005981, LIVER2006006, LIVER2006251, LIVER2006410, LIVER2006469, LIVER2006644, LIVER2006764, LIVER2007415, LIVER2007548, LIVER2007568, LIVER2007721, LIVER2008053, LIVER2008465, LIVER2008473, LIVER2008580, LIVER2008706,

LIVER2008751, LIVER2008945, LIVER2008998, LIVER2009110, LIVER2009118, LIVER2009554, LYMPB1000158, LYMPB2000083, MAMGL1000017, MAMGL1000083, MAMGL1000096, MAMGL1000132, MAMGL1000151, MAMGL1000178, MAMGL1000182, MAMGL1000184, MESAN1000032, MESAN1000035, MESAN1000079, MESAN1000121, MESAN1000126, MESAN1000147, MESAN1000180, MESAN2000023, MESAN2000026, MESAN2000067, MESAN2000167, MESAN2000264, MESAN2000337, MESAN2000457, MESAN2000462, MESAN2000572, MESAN2000608, MESAN2000620, MESAN2000711, MESAN2000815, MESAN2000894, MESAN2001154, MESAN2001627, MESAN2001770, MESAN2001979, MESAN2001980, MESAN2002113, MESAN2002122, MESAN2002147, MESAN2002186, MESAN2002424, MESAN2002687, MESAN2002709, MESAN2002978,

MESAN2003035, MESAN2003037, MESAN2003039, MESAN2003058, MESAN2003101, MESAN2003190, MESAN2003322, MESAN2003490, MESAN2003529, MESAN2003646, MESAN2003662, MESAN2003709, MESAN2003851, MESAN2004138, MESAN2004288, MESAN2004575, MESAN2005284, MESAN2005303, MESAN2005371, MESAN2005724, MESAN2005808, MESAN2005811, MESAN2005957, MESAN2006022, MESAN2006043, MESAN2006328, MESAN2006401, MESAN2006580, MESAN2006599, MESAN2006743, MESAN2006953, MESAN2006971, MESAN2007032, MESAN2007440, MESAN2007597, MESAN2008222, MESAN2008334, MESAN2008415, MESAN2008460, MESAN2008536, MESAN2008729, MESAN2008821, MESAN2008926, MESAN2008936, MESAN2009019, MESAN2009081, MESAN2009105, MESAN2009311, MESAN2009418, MESAN2009503,

MESAN2009522, MESAN2010031, MESAN2010114, MESAN2010312, MESAN2010321, MESAN2010664, MESAN2010681, MESAN2011545, MESAN2011597, MESAN2011627, MESAN2011632, MESAN2011977, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013908, MESAN2013936, MESAN2014192, MESAN2014295, MESAN2014298, MESAN2014624, MESAN2015277, MESAN2015365, MESAN2015391, MESAN2015401, MESAN2015501, MESAN2015708, MESAN2016304, MESAN2016409, MESAN2016552, MESAN2016965, MESAN2017133, MESAN2017152, MESAN2017373, MESAN2017417, MESAN2018209, MESAN2018576, MESAN2018670, MESAN2018699, MESTC1000042, MESTC1000147,

MESTC2000150, MESTC2000153, MESTC2000170, N1ESE2000698, NB9N41000011, NB9N41000047, NB9N41000135, NB9N41000142, NB9N41000146, NB9N42000122, NB9N42000196, NB9N42000281, NB9N42000314, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062, NHNPC2000606, NHNPC2000877, NHNPC2001223, NHNPC2001312,

NHNPC2001816, NHNPC2001931, NHNPC2002565, NHNPC2002749, NOVAR1000015, NOVAR2000038, NOVAR2000136, NOVAR2000352, NOVAR2000412, NOVAR2000710, NOVAR2000962, NOVAR2001108, NOVAR2001620, NOVAR2001783, NOVAR2001876, NOVAR2002039, NT2NE1000004, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000222, NT2NE2000284, NT2NE2000299, NT2NE2000353, NT2NE2000356, NT2NE2000369, NT2NE2000374, NT2NE2000386, NT2NE2000390, NT2NE2000392, NT2NE2000455, NT2NE2000470, NT2NE2000488, NT2NE2000517, NT2NE2000536,

NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000864, NT2NE2000909, NT2NE2000913, NT2NE2000951, NT2NE2000980, NT2NE2001000, NT2NE2001021, NT2NE2001040, NT2NE2001049, NT2NE2001142, NT2NE2001156, NT2NE2001180, NT2NE2001181, NT2NE2001188, NT2NE2001191, NT2NE2001247, NT2NE2001252, NT2NE2001324, NT2NE2001326, NT2NE2001337, NT2NE2001364, NT2NE2001372, NT2NE2001397, NT2NE2001403, NT2NE2001428, NT2NE2001432, NT2NE2001442, NT2NE2001524, NT2NE2001530, NT2NE2001617, NT2NE2001623, NT2NE2001626, NT2NE2001634, NT2NE2001655, NT2NE2001660, NT2NE2001666, NT2NE2001677, NT2NE2001698, NT2NE2001723, NT2NE2001725,

NT2NE2001874, NT2NE2001889, NT2NE2001923, NT2NE2002100, NT2NE2002162, NT2NE2002186, NT2NE2002285, NT2NE2002311, NT2NE2002620, NT2NE2002651, NT2NE2002732, NT2NE2002768, NT2NE2002791, NT2NE2002856, NT2NE2003150, NT2NE2003185, NT2NE2003252, NT2NE2003280, NT2NE2003308, NT2NE2003309, NT2NE2003315, NT2NE2003393, NT2NE2003408, NT2NE2003457, NT2NE2003485, NT2NE2003540, NT2NE2003571, NT2NE2003705, NT2NE2003752, NT2NE2004378, NT2NE2004490, NT2NE2004519, NT2NE2004606, NT2NE2004716, NT2NE2004740, NT2NE2004787, NT2NE2004916, NT2NE2005035, NT2NE2005223, NT2NE2005276, NT2NE2005317, NT2NE2005323, NT2NE2005358, NT2NE2005371, NT2NE2005395, NT2NE2005441, NT2NE2005517, NT2NE2005537, NT2NE2005555, NT2NE2005588,

NT2NE2005676, NT2NE2005720, NT2NE2005784, NT2NE2005804, NT2NE2005821, NT2NE2005876, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006458, NT2NE2006531, NT2NE2006659, NT2NE2006784, NT2NE2006813, NT2NE2006894, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008803, NT2NE2008867, NT2NE2008961, NT2NE2008997, NT2NE2009138, NT2NE2009295,

NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2011033, NT2NE2011036, NT2NE2011119, NT2NE2011154, NT2NE2011221, NT2NE2011411, NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012505, NT2NE2012533, NT2NE2012603, NT2NE2012747, NT2NE2012847, NT2NE2012928, NT2NE2013019, NT2NE2013081, NT2NE2013189, NT2NE2013217, NT2NE2013547, NT2NE2014013, NT2NE2014028, NT2NE2014113, NT2NE2014176, NT2NE2014221,

NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2015974, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2018165, NT2NE2018176, NT2NE2018273, NT2NE2018376, NT2NE2018472, NT2NE2018594, NT2NE2018739, NT2NE2018916, NT2NE2019056, NT2NE2019076,

NT2NE2019099, NT2RI1000016, NT2RI1000035, NT2RI1000127, NT2RI1000164, NT2RI1000172, NT2RI2000004, NT2RI2000007, NT2RI2000116, NT2RI2000117, NT2RI2000128, NT2RI2000167, NT2RI2000173, NT2RI2000187, NT2RI2000270, NT2RI2000276, NT2RI2000282, NT2RI2000313, NT2RI2000341, NT2RI2000344, NT2RI2000348, NT2RI2000480, NT2RI2000575, NT2RI2000588, NT2RI2000597, NT2RI2000669, NT2RI2000671, NT2RI2000688, NT2RI2000704, NT2RI2000738, NT2RI2000775, NT2RI2000822, NT2RI2000829, NT2RI2000841, NT2RI2000865, NT2RI2000932, NT2RI2000974, NT2RI2000987, NT2RI2001010, NT2RI2001017, NT2RI2001030, NT2RI2001054, NT2RI2001083, NT2RI2001178, NT2RI2001235, NT2RI2001244, NT2RI2001307, NT2RI2001385, NT2RI2001409, NT2RI2001410,

NT2RI2001425, NT2RI2001449, NT2RI2001450, NT2RI2001474, NT2RI2001519, NT2RI2001595, NT2RI2001624, NT2RI2001726, NT2RI2001769, NT2RI2001782, NT2RI2001836, NT2RI2001846, NT2RI2001866, NT2RI2001952, NT2RI2002022, NT2RI2002041, NT2RI2002120, NT2RI2002153, NT2RI2002179, NT2RI2002252, NT2RI2002260, NT2RI2002270, NT2RI2002334, NT2RI2002359, NT2RI2002391, NT2RI2002447, NT2RI2002481, NT2RI2002507, NT2RI2002517, NT2RI2002530, NT2RI2002540, NT2RI2002549, NT2RI2002564, NT2RI2002592, NT2RI2002602, NT2RI2002699, NT2RI2002729, NT2RI2002819, NT2RI2002822, NT2RI2002865, NT2RI2002923, NT2RI2002926, NT2RI2002958, NT2RI2002970, NT2RI2003011, NT2RI2003067, NT2RI2003184, NT2RI2003222, NT2RI2003301, NT2RI2003360,

NT2RI2003361, NT2RI2003383, NT2RI2003392, NT2RI2003407, NT2RI2003419, NT2RI2003481, NT2RI2003487, NT2RI2003655, NT2RI2003667, NT2RI2003751, NT2RI2003884, NT2RI2003973, NT2RI2004059, NT2RI2004078, NT2RI2004079, NT2RI2004093, NT2RI2004136, NT2RI2004157, NT2RI2004190, NT2RI2004230, NT2RI2004284, NT2RI2004290, NT2RI2004304, NT2RI2004312, NT2RI2004381, NT2RI2004442, NT2RI2004468, NT2RI2004606, NT2RI2004618, NT2RI2004783, NT2RI2004818, NT2RI2004840, NT2RI2004884, NT2RI2004916, NT2RI2004962, NT2RI2004984, NT2RI2004985, NT2RI2005048, NT2RI2005069, NT2RI2005087, NT2RI2005115, NT2RI2005130, NT2RI2005150, NT2RI2005239, NT2RI2005246, NT2RI2005315, NT2RI2005353, NT2RI2005358, NT2RI2005368, NT2RI2005382,

NT2RI2005473, NT2RI2005520, NT2RI2005564, NT2RI2005579, NT2RI2005621, NT2RI2005628, NT2RI2005670, NT2RI2005710, NT2RI2005772, NT2RI2005814, NT2RI2005816, NT2RI2005851, NT2RI2005966, NT2RI2006071, NT2RI2006081, NT2RI2006126, NT2RI2006127, NT2RI2006183, NT2RI2006257, NT2RI2006271, NT2RI2006294, NT2RI2006345, NT2RI2006403, NT2RI2006445, NT2RI2006487, NT2RI2006565, NT2RI2006640, NT2RI2006659, NT2RI2006662, NT2RI2006667, NT2RI2006679, NT2RI2006682, NT2RI2006686, NT2RI2006703, NT2RI2006716, NT2RI2006762, NT2RI2006788, NT2RI2006838, NT2RI2006853, NT2RI2006973, NT2RI2007046, NT2RI2007048, NT2RI2007084, NT2RI2007096, NT2RI2007116, NT2RI2007214, NT2RI2007277, NT2RI2007303, NT2RI2007334, NT2RI2007377,

NT2RI2007386, NT2RI2007406, NT2RI2007439, NT2RI2007507, NT2RI2007593, NT2RI2007629, NT2RI2007751, NT2RI2007754, NT2RI2007827, NT2RI2007877, NT2RI2007884, NT2RI2007891, NT2RI2007956, NT2RI2007965, NT2RI2007987, NT2RI2008050, NT2RI2008141, NT2RI2008204, NT2RI2008221, NT2RI2008233, NT2RI2008455, NT2RI2008481, NT2RI2008502, NT2RI2008566, NT2RI2008598, NT2RI2008622, NT2RI2008714, NT2RI2008791, NT2RI2008801, NT2RI2008812, NT2RI2008841, NT2RI2008952, NT2RI2009005, NT2RI2009101, NT2RI2009144, NT2RI2009151, NT2RI2009194, NT2RI2009215, NT2RI2009239, NT2RI2009259, NT2RI2009269, NT2RI2009289, NT2RI2009301, NT2RI2009402, NT2RI2009406, NT2RI2009517, NT2RI2009585, NT2RI2009595, NT2RI2009855, NT2RI2010283,

NT2RI2010508, NT2RI2010795, NT2RI2011422, NT2RI2011450, NT2RI2011683, NT2RI2011803, NT2RI2012659, NT2RI2012990, NT2RI2013345, NT2RI2013373, NT2RI2014247, NT2RI2014551, NT2RI2014683, NT2RI2014733, NT2RI2015239, NT2RI2016128, NT2RI2016134, NT2RI2017529, NT2RI2018311, NT2RI2018363, NT2RI2018883, NT2RI2018950, NT2RI2019751, NT2RI2019826, NT2RI2020390, NT2RI2020703, NT2RI2021463, NT2RI2021625, NT2RI2022468, NT2RI2022584, NT2RI2023303, NT2RI2023671, NT2RI2024008, NT2RI2024460, NT2RI2024496, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027323, NT2RI2027396, NT2RI2027484, NT2RI2027975,

NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001445, NT2RI3001458, NT2RI3001573, NT2RI3001967, NT2RI3002303, NT2RI3002557, NT2RI3002842, NT2RI3003027, NT2RI3003031, NT2RI3003095, NT2RI3003162, NT2RI3003362, NT2RI3003382, NT2RI3003409, NT2RI3003738, NT2RI3003925, NT2RI3004133, NT2RI3004381, NT2RI3004510, NT2RI3005202, NT2RI3005403, NT2RI3005416, NT2RI3005861, NT2RI3005923, NT2RI3005928, NT2RI3006132, NT2RI3006171, NT2RI3006376, NT2RI3006666, NT2RI3006673, NT2RI3006796, NT2RI3007065, NT2RI3007095, NT2RI3007158, NT2RI3007167, NT2RI3007213, NT2RI3007291, NT2RI3007443,

NT2RI3007684, NT2RI3007757, NT2RI3007878, NT2RI3007978, NT2RI3008055, NT2RI3008162, NT2RI3008179, NT2RI3008228, NT2RI3008442, -NT2RI3008652, NT2RI3008697, NT2RI3008974, NT2RI3009480, NT2RI3009524, NT2RP6000005, NT2RP6000008, NT2RP6000032, NT2RP6000033, NT2RP6000059, NT2RP6000060, NT2RP6000077, NT2RP6000086, NT2RP6000119, NT2RP6000127, NT2RP6000140, NT2RP6000150, NT2RP7000041, NT2RP7000086, NT2RP7000109, NT2RP7000112, NT2RP7000311, NT2RP7000359, NT2RP7000364, NT2RP7000380, NT2RP7000391, NT2RP7000425, NT2RP7000579, NT2RP7000812, NT2RP7000926, NT2RP7001074, NT2RP7001306, NT2RP7001319, NT2RP7001335, NT2RP7001364, NT2RP7001475, NT2RP7001532, NT2RP7001591, NT2RP7001602, NT2RP7001856, NT2RP7002064,

NT2RP7002151, NT2RP7002163, NT2RP7002243, NT2RP7002282, NT2RP7002376, NT2RP7002478, NT2RP7002554, NT2RP7002594, NT2RP7002603, NT2RP7002650, NT2RP7002829, NT2RP7002841, NT2RP7002875, NT2RP7002978, NT2RP7003025, NT2RP7003050, NT2RP7003055, NT2RP7003084, NT2RP7003091, NT2RP7003107, NT2RP7003134, NT2RP7003148, NT2RP7003439, NT2RP7003511, NT2RP7003632, NT2RP7004027, NT2RP7004080, NT2RP7004114, NT2RP7004123, NT2RP700434.8, NT2RP7004358, NT2RP7004396, NT2RP7004428, NT2RP7004481, NT2RP7004541, NT2RP7004559, NT2RP7004656, NT2RP7004722, NT2RP7004766, NT2RP7004790, NT2RP7004884, NT2RP7004915, NT2RP7004925, NT2RP7004961, NT2RP7004964, NT2RP7004975, NT2RP7005118, NT2RP7005205, NT2RP7005219, NT2RP7005323,

NT2RP7005468, NT2RP7005493, NT2RP7005513, NT2RP7005629, NT2RP7005631, NT2RP7005669, NT2RP7005846, NT2RP7006075, NT2RP7006141, NT2RP7006160, NT2RP7006162, NT2RP7006263, NT2RP7006374, NT2RP7006395, NT2RP7006408, NT2RP7006457, NT2RP7006490, NT2RP7006601, NT2RP7006619, NT2RP7006621, NT2RP7006636, NT2RP7006701, NT2RP7006853, NT2RP7006886, NT2RP7006887, NT2RP7006980, NT2RP7006986, NT2RP7006995, NT2RP7007177, NT2RP7007221, NT2RP7007226, NT2RP7007269, NT2RP7007359, NT2RP7007406, NT2RP7007480, NT2RP7007504, NT2RP7007530, NT2RP7007594, NT2RP7007610, NT2RP7007617, NT2RP7007766, NT2RP7007925, NT2RP7007975, NT2RP7008013, NT2RP7008015, NT2RP7008133, NT2RP7008137, NT2RP7008142, NT2RP7008144, NT2RP7008161,

NT2RP7008360, NT2RP7008396, NT2RP7008406, NT2RP7008435, NT2RP7008441, NT2RP7008454, NT2RP7008487, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009097, NT2RP7009168, NT2RP7009215, NT2RP7009225, NT2RP7009236, NT2RP7009253, NT2RP7009267, NT2RP7009363, NT2RP7009397, NT2RP7009429, NT2RP7009439, NT2RP7009466, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010612, NT2RP7010817, NT2RP7011086, NT2RP7011132, NT2RP7011268, NT2RP7011318, NT2RP7011452,

NT2RP7011570, NT2RP7011721, NT2RP7011909, NT2RP7012291, NT2RP7012516, NT2RP7012776, NT2RP7013002, NT2RP7013374, NT2RP7013499, NT2RP7013573, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871,

NT2RP7019064, NT2RP7019135, NT2RP7019273, NT2RP7019367, NT2RP7019401, NT2RP7019445, NT2RP7019543, NT2RP7019682, NT2RP7019835, NT2RP7020112, NT2RP7020343, NT2RP7020379, NT2RP8000137, NT2RP8000296, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8008057, NT2RP8009248, NTONG1000006, NTONG1000047, NTONG1000052, NTONG1000075, NTONG1000087, NTONG1000123, NTONG1000130, NTONG1000161, NTONG1000172, NTONG1000182, NTONG1000213, NTONG1000214, NTONG1000247, NTONG1000257, NTONG1000264, NTONG2000040,

NTONG2000107, NTONG2000231, NTONG2000363, NTONG2000492, NTONG2000499, NTONG2000531, NTONG2000583, NTONG2000858, NTONG2000876, NTONG2000966, NTONG2001014, NTONG2001079, NTONG2001157, NTONG2001173, NTONG2001220, NTONG2001362, NTONG2001428, NTONG2001532, NTONG2001612, NTONG2001619, NTONG2001642, NTONG2001657, NTONG2001676, NTONG2002278, NTONG2002807, NTONG2002948, NTONG2002970, NTONG2002985, NTONG2003158, NTONG2003210, NTONG2003316, NTONG2003409, NTONG2003515, NTONG2003805, NTONG2003839, NTONG2003852, NTONG2003897, NTONG2004095, NTONG2004308, NTONG2004521, NTONG2004614, NTONG2004690, NTONG2004806, NTONG2004829, NTONG2004918, NTONG2004991, NTONG2005062, NTONG2005086, NTONG2005137, NTONG2005153,

NTONG2005265, NTONG2005277, NTONG2005363, NTONG2005373, NTONG2005480, NTONG2005497, NTONG2005577, NTONG2005657, NTONG2005822, NTONG2005897, NTONG2005969, NTONG2006099, NTONG2006187, NTONG2006324, NTONG2006354, NTONG2006440, NTONG2006501, NTONG2006646, NTONG2006709, NTONG2006783, NTONG2007020, NTONG2007028, NTONG2007034, NTONG2007249, NTONG2007517, NTONG2007522, NTONG2007756, NTONG2008093, NTONG2008143, NTONG2008365, NTONG2008522, NTONG2008672, NTONG2008862, NTONG2008939, NTONG2009060, NTONG2009068, NTONG2009229, NTONG2009233, NTONG2009468, OCBBF1000016, OCBBF1000030, OCBBF1000042, OCBBF1000049, OCBBF1000054, OCBBF1000067, OCBBF1000080, OCBBF1000085, OCBBF1000091, OCBBF1000104, OCBBF1000114,

OCBBF1000118, OCBBF1000119, OCBBF1000122, OCBBF1000130, OCBBF1000138, OCBBF1000145, OCBBF1000146, OCBBF1000153, OCBBF1000175, OCBBF1000192, OCBBF1000254, OCBBF2000126, OCBBF2000178, OCBBF2000231, OCBBF2000277, OCBBF2000287, OCBBF2000323, OCBBF2000452, OCBBF2000522, OCBBF2000677, OCBBF2000703, OCBBF2000712, OCBBF2000719, OCBBF2000824, OCBBF2000904, OCBBF2000998, OCBBF2001075, OCBBF2001101, OCBBF2001124, OCBBF2001176, OCBBF2001196, OCBBF2001307, OCBBF2001323, OCBBF2001389, OCBBF2001402, OCBBF2001473, OCBBF2001494, OCBBF2001527, OCBBF2001639, OCBBF2001681, OCBBF2001706, OCBBF2001749, OCBBF2001910, OCBBF2001931, OCBBF2001961, OCBBF2001983, OCBBF2002015, OCBBF2002124, OCBBF2002161, OCBBF2002290,

OCBBF2002357, OCBBF2002376, OCBBF2002615, OCBBF2002626, OCBBF2002663, OCBBF2002805, OCBBF2002920, OCBBF2002980, OCBBF2003028, OCBBF2003091, OCBBF2003246, OCBBF2003327, OCBBF2003543, OCBBF2003593, OCBBF2003744, OCBBF2004038, OCBBF2004104, OCBBF2004147, OCBBF2004168, OCBBF2004235, OCBBF2004259, OCBBF2004385, OCBBF2004418, OCBBF2004478, OCBBF2004482, OCBBF2004533, OCBBF2004567, OCBBF2004612, OCBBF2004647, OCBBF2004669, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005546, OCBBF2005843, OCBBF2005901, OCBBF2006030, OCBBF2006058,

OCBBF2006113, OCBBF2006148, OCBBF2006154, OCBBF2006172, OCBBF2006214, OCBBF2006241, OCBBF2006313, OCBBF2006388, OCBBF2006438, OCBBF2006567, OCBBF2006624, OCBBF2006639, OCBBF2006660, OCBBF2006764, OCBBF2006849, OCBBF2006859, OCBBF2006972, OCBBF2007039, OCBBF2007051, OCBBF2007068, OCBBF2007114, OCBBF2007121, OCBBF2007184, OCBBF2007194, OCBBF2007196, OCBBF2007238, OCBBF2007414, OCBBF2007428, OCBBF2007478, OCBBF2007485, OCBBF2007520, OCBBF2007555, OCBBF2007610, OCBBF2007622, OCBBF2007756, OCBBF2007762, OCBBF2007931, OCBBF2007946, OCBBF2008005, OCBBF2008116, OCBBF2008138, OCBBF2008283, OCBBF2008330, OCBBF2008340, OCBBF2008366, OCBBF2008466, OCBBF2008483, OCBBF2008511, OCBBF2008520, OCBBF2008569,

OCBBF2008586, OCBBF2008640, OCBBF2008691, OCBBF2008701, OCBBF2008724, OCBBF2008760, OCBBF2008768, OCBBF2008775, OCBBF2008790, OCBBF2008814, OCBBF2008822, OCBBF2009095, OCBBF2009115, OCBBF2009301, OCBBF2009352, OCBBF2009391, OCBBF2009536, OCBBF2009571, OCBBF2009583, OCBBF2009603, OCBBF2009788, OCBBF2009920, OCBBF2009926, OCBBF2010040, OCBBF2010140, OCBBF2010281, OCBBF2010313, OCBBF2010404, OCBBF2010416, OCBBF2010420, OCBBF2010557, OCBBF2010604, OCBBF2010709, OCBBF2010792, OCBBF2010841, OCBBF2010843, OCBBF2010858, OCBBF2010863, OCBBF2010871, OCBBF2010931, OCBBF2010945, OCBBF2010978, OCBBF2010989, OCBBF2011073, OCBBF2011137, OCBBF2011160, OCBBF2011177, OCBBF2011228, OCBBF2011232, OCBBF2011283,

OCBBF2011311, OCBBF2011625, OCBBF2011669, OCBBF2011685, OCBBF2011706, OCBBF2011722, OCBBF2011767, OCBBF2011855, OCBBF2011872, OCBBF2011887, OCBBF2011897, OCBBF2011914, OCBBF2011960, OCBBF2011981, OCBBF2012028, OCBBF2012039, OCBBF2012095, OCBBF2012139, OCBBF2012191, OCBBF2012262, OCBBF2012320, OCBBF2012436, OCBBF2012525, OCBBF2012678, OCBBF2012704, OCBBF2012714, OCBBF2012755, OCBBF2012812, OCBBF2012881, OCBBF2012908, OCBBF2012936, OCBBF2012990, OCBBF2013011, OCBBF2013049, OCBBF2013091, OCBBF2013123, OCBBF2013127, OCBBF2013149, OCBBF2013208, OCBBF2013285, OCBBF2013319, OCBBF2013324, OCBBF2013366, OCBBF2013481, OCBBF2013507, OCBBF2013721, OCBBF2013789, OCBBF2013843, OCBBF2013883, OCBBF2013926,

OCBBF2014052, OCBBF2014064, OCBBF2014089, OCBBF2014322, OCBBF2014386, OCBBF2014576, OCBBF2014707, OCBBF2014745, OCBBF2014828, OCBBF2014880, OCBBF2014928, OCBBF2015031, OCBBF2015081, OCBBF2015115, OCBBF2015232, OCBBF2015233, OCBBF2015285, OCBBF2015335, OCBBF2015503, OCBBF2015506, OCBBF2015599, OCBBF2015645, OCBBF2015659, OCBBF2015751, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016841, OCBBF2017035, OCBBF2017055, OCBBF2017069, OCBBF2017306, OCBBF2017325, OCBBF2017489, OCBBF2017516, OCBBF2017665, OCBBF2017882, OCBBF2017888, OCBBF2018012, OCBBF2018014, OCBBF2018084, OCBBF2018167,

OCBBF2018206, OCBBF2018229, OCBBF2018234, OCBBF2018362, OCBBF2018380, OCBBF2018581, OCBBF2018663, OCBBF2018687, OCBBF2018828, OCBBF2018934, OCBBF2019108, OCBBF2019195, OCBBF2019327, OCBBF2019684, OCBBF2019761, OCBBF2019823, OCBBF2019950, OCBBF2020318, OCBBF2020343, OCBBF2020453, OCBBF2020741, OCBBF2020801, OCBBF2020825, OCBBF2020838, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2025028, OCBBF2025115, OCBBF2025198, OCBBF2025451, OCBBF2025458, OCBBF2025527, OCBBF2025631, OCBBF2025730, OCBBF2026025,

OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481, OCBBF2027511, OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906,

OCBBF2035110, OCBBF2035214, OCBBF2035226, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036743, OCBBF2036752, OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000372, OCBBF3000380, OCBBF3000743, OCBBF3000830, OCBBF3001076, OCBBF3001202, OCBBF3001333, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004487, OCBBF3004972, OCBBF3005330, OCBBF3005597, OCBBF3005843,

OCBBF3006802, OCBBF3006986, OCBBF3007078, OCBBF3007704, OCBBF3008230, OCBBF3008392, OCBBF3008835, OCBBF3009192, OCBBF3009279, PANCR1000012, PANCR1000021, PANCR1000081, PANCR1000086, PANCR1000091, PANCR1000185, PEBLM1000024, PEBLM1000029, PEBLM1000034, PEBLM1000062, PEBLM1000068, PEBLM1000071, PEBLM1000083, PEBLM1000147, PEBLM1000174, PEBLM1000180, PEBLM2000112, PEBLM2000126, PEBLM2000147, PEBLM2000170, PEBLM2000180, PEBLM2000213, PEBLM2000248, PEBLM2000270, PEBLM2000321, PEBLM2000333, PEBLM2000395, PEBLM2000479, PEBLM2000502, PEBLM2001312, PEBLM2001465, PEBLM2001488, PEBLM2002432, PEBLM2002455, PEBLM2002594, PEBLM2002887, PEBLM2003426, PEBLM2003765, PEBLM2003935, PEBLM2004015, PEBLM2004216,

PEBLM2004290, PEBLM2004497, PEBLM2005282, PEBLM2005615, PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006366, PEBLM2006709, PEBLM2006912, PEBLM2007112, PEBLM2007140, PEBLM2007188, PEBLM2007296, PEBLM2007437, PEBLM2007447, PEBLM2007598, PEBLM2007832, PEBLM2007834, PEBLM2008106, PEBLM2008576, PEBLM2008605, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766,

PERIC2003001, PERIC2003090, PERIC2003287, PERIC2003349, PERIC2003452, PERIC2003699, PERIC2003720, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000037, PLACE5000058, PLACE5000066, PLACE5000070, PLACE5000080, PLACE5000105, PLACE5000115, PLACE5000116, PLACE5000129, PLACE5000139, PLACE5000153, PLACE5000171,

PLACE5000172, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000296, PLACE6000314, PLACE6000325, PLACE6000348, PLACE6000371, PLACE6000424, PLACE6000426, PLACE6000429, PLACE6000450, PLACE6000523, PLACE6000550, PLACE6000552, PLACE6000555, PLACE6000619, PLACE6000630, PLACE6000953, PLACE6001118, PLACE6001153, PLACE6001185, PLACE6001262, PLACE6001294, PLACE6001443, PLACE6001536, PLACE6001557, PLACE6001712, PLACE6001740, PLACE6001823, PLACE6001886, PLACE6001923, PLACE6001925, PLACE6001933, PLACE6002011,

PLACE6002016, PLACE6002056, PLACE6002157, PLACE6002312, PLACE6002323, PLACE6002345, PLACE6002419, PLACE6002462, PLACE6002647, PLACE6002668, PLACE6002692, PLACE6002699, PLACE6002710, PLACE6002871, PLACE6002949, PLACE6003004, PLACE6003094, PLACE6003109, PLACE6003204, PLACE6003218, PLACE6003372, PLACE6003399, PLACE6003539, PLACE6003621, PLACE6003705, PLACE6003740, PLACE6003745, PLACE6003771, PLACE6003796, PLACE6003839, PLACE6003850, PLACE6004005, PLACE6004101, PLACE6004219, PLACE6004336, PLACE6004368, PLACE6004380, PLACE6004396, PLACE6004454, PLACE6004464, PLACE6004491, PLACE6004663, PLACE6004687, PLACE6004738, PLACE6004931, PLACE6004966, PLACE6004993, PLACE6005007, PLACE6005029, PLACE6005108,

PLACE6005234, PLACE6005283, PLACE6005294, PLACE6005328, PLACE6005351, PLACE6005423, PLACE6005487, PLACE6005535, PLACE6005546, PLACE6005559, PLACE6005579, PLACE6005691, PLACE6005786, PLACE6006042, PLACE6006077, PLACE6006137, PLACE6006158, PLACE6006186, PLACE6006217, PLACE6006222, PLACE6006266, PLACE6006287, PLACE6006394, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444,

PLACE6008453, PLACE6008640, PLACE6008750, PLACE6008768, PLACE6008824, PLACE6008989, PLACE6009006, PLACE6009008, PLACE6009228, PLACE6009237, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011102, PLACE6011156, PLACE6011211, PLACE6011260, PLACE6011627, PLACE6011774, PLACE6011881, PLACE6011907, PLACE6011970, PLACE6011975, PLACE6012108, PLACE6012168, PLACE6012297, PLACE6012308, PLACE6012574, PLACE6012908, PLACE6012936,

PLACE6012942, PLACE6013232, PLACE6013288, PLACE6013386, PLACE6013400, PLACE6013650, PLACE6013672, PLACE6013883, PLACE6013884, PLACE6013885, PLACE6014043, PLACE6014064, PLACE6014516, PLACE6014882, PLACE6015211, PLACE6015322, PLACE6015445, PLACE6015460, PLACE6015591, PLACE6015729, PLACE6015731, PLACE6015939, PLACE6016030, PLACE6016093, PLACE6016105, PLACE6016160, PLACE6016210, PLACE6016254, PLACE6016942, PLACE6016984, PLACE6017002, PLACE6017431, PLACE6017564, PLACE6017578, PLACE6017626, PLACE6017701, PLACE6017714, PLACE6018107, PLACE6018187, PLACE6018235, PLACE6018441, PLACE6018487, PLACE6018497, PLACE6018769, PLACE6018834, PLACE6018843, PLACE6018863, PLACE6018938, PLACE6019385, PLACE6019542,

PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7005840, PLACE7006051, PLACE7006090, PLACE7006240, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000110, PROST1000120,

PROST1000123, PROST1000136, PROST1000145, PROST1000152, PROST1000167, PROST1000184, PROST1000215, PROST1000217, PROST1000246, PROST1000262, PROST1000272, PROST1000298, PROST1000322, PROST1000334, PROST1000343, PROST1000362, PROST1000377, PROST1000383, PROST1000412, PROST1000419, PROST1000451, PROST1000480, PROST1000485, PROST1000493, PROST1000512, PROST1000526, PROST1000527, PROST1000528, PROST1000536, PROST1000559, PROST1000575, PROST2000036, PROST2000053, PROST2000067, PROST2000105, PROST2000143, PROST2000162, PROST2000165, PROST2000176, PROST2000203, PROST2000206, PROST2000212, PROST2000241, PROST2000267, PROST2000273, PROST2000274, PROST2000277, PROST2000337, PROST2000432, PROST2000452,

PROST2000463, PROST2000470, PROST2000505, PROST2000572, PROST2000717, PROST2000760, PROST2000761, PROST2000835, PROST2000893, PROST2000961, PROST2001002, PROST2001116, PROST2001138, PROST2001213, PROST2001283, PROST2001414, PROST2001415, PROST2001465, PROST2001540, PROST2001599, PROST2001611, PROST2001636, PROST2001739, PROST2001796, PROST2001805, PROST2001823, PROST2001899, PROST2001998, PROST2002078, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002651, PROST2002682, PROST2002685, PROST2002722, PROST2002736, PROST2002842, PROST2002897, PROST2002906, PROST2002927,

PROST2002960, PROST2003102, PROST2003117, PROST2003210, PROST2003302, PROST2003303, PROST2003324, PROST2003340, PROST2003396, PROST2003428, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004332, PROST2004481, PROST2004526, PROST2004570, PROST2004727, PROST2004742, PROST2004744, PROST2004817, PROST2005039, PROST2005067, PROST2005076, PROST2005093, PROST2005121, PROST2005131, PROST2005143, PROST2005228, PROST2005272, PROST2005285, PROST2005426,

PROST2005466, PROST2005630, PROST2005683, PROST2005788, PROST2005793, PROST2005880, PROST2005886, PROST2005904, PROST2005919, PROST2005943, PROST2006008, PROST2006020, PROST2006030, PROST2006196, PROST2006201, PROST2006260, PROST2006450, PROST2006491, PROST2006510, PROST2006536, PROST2006579, PROST2006688, PROST2006766, PROST2006782, PROST2006933, PROST2006988, PROST2007122, PROST2007200, PROST2007237, PROST2007248, PROST2007289, PROST2007328, PROST2007382, PROST2007389, PROST2007416, PROST2007528, PROST2007818, PROST2007950, PROST2007956, PROST2007974, PROST2007988, PROST2008079, PROST2008088, PROST2008243, PROST2008245, PROST2008268, PROST2008271, PROST2008360, PROST2008447, PROST2008468,

PROST2008472, PROST2008516, PROST2008724, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010318, PROST2010326, PROST2010382, PROST2010400, PROST2010545, PROST2010606, PROST2010782, PROST2010885, PROST2011012, PROST2011038, PROST2011297, PROST2011410, PROST2011439, PROST2011482, PROST2011631, PROST2011660, PROST2012005, PROST2012016, PROST2012088, PROST2012140, PROST2012157, PROST2012190, PROST2012249, PROST2012353,

PROST2012400, PROST2012504, PROST2012542, PROST2012550, PROST2012740, PROST2012780, PROST2012888, PROST2012890, PROST2013032, PROST2013053, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015198, PROST2015243, PROST2015246, PROST2015251, PROST2015287, PROST2015332, PROST2015457, PROST2015464, PROST2015537, PROST2015545, PROST2015551, PROST2015636, PROST2015710, PROST2015756, PROST2015877, PROST2015900, PROST2015924, PROST2015932, PROST2016195, PROST2016246, PROST2016351, PROST2016379, PROST2016444, PROST2016462, PROST2016499,

PROST2016512, PROST2016566, PROST2016582, PROST2016668, PROST2016684, PROST2016777, PROST2016829, PROST2016860, PROST2016918, PROST2016980, PROST2017098, PROST2017128, PROST2017367, PROST2017413, PROST2017441, PROST2017529, PROST2017578, PROST2017612, PROST2017617, PROST2017692, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019296, PROST2019398, PROST2019487, PROST2019781, PUAEN1000039, PUAEN1000081, PUAEN1000085, PUAEN1000087, PUAEN1000121, PUAEN1000147, PUAEN1000175,

PUAEN1000275, PUAEN1000276, PUAEN1000288, PUAEN2000080, PUAEN2000175, PUAEN2000312, PUAEN2000394, PUAEN2000497, PUAEN2000594, PUAEN2000684, PUAEN2000942, PUAEN2001260, PUAEN2001345, PUAEN2001526, PUAEN2001882, PUAEN2002473, PUAEN2002489, PUAEN2002552, PUAEN2002568, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005588, PUAEN2005930, PUAEN2006029, PUAEN2006328, PUAEN2006335, PUAEN2006578, PUAEN2006639, PUAEN2006761, PUAEN2007350, PUAEN2007785, PUAEN2007898, PUAEN2008123, PUAEN2008228, PUAEN2008314, PUAEN2008515,

PUAEN2008815, PUAEN2008939, PUAEN2008980, PUAEN2009174, PUAEN2009348, PUAEN2009534, PUAEN2009655, PUAEN2009795, PUAEN2009852, PUAEN2010346, PUAEN2010824, RECTM1000141, RECTM2000220, RECTM2000349, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000065, SALGL1000171, SKMUS1000030, SKMUS1000064, SKMUS1000067, SKMUS1000102, SKMUS1000104, SKMUS1000110, SKMUS1000118, SKMUS1000124, SKMUS1000129, SKMUS1000177, SKMUS2000020, SKMUS2000061, SKMUS2000074, SKMUS2000078, SKMUS2000117, SKMUS2000137, SKMUS2000198, SKMUS2000271, SKMUS2000287, SKMUS2000317,

SKMUS2000330, SKMUS2000339, SKMUS2000343, SKMUS2000365, SKMUS2000380, SKMUS2000390, SKMUS2000416, SKMUS2000458, SKMUS2000467, SKMUS2000468, SKMUS2000515, SKMUS2000690, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000873, SKMUS2000894, SKMUS2000898, SKMUS2000945, SKMUS2000973, SKMUS2000980, SKMUS2001008, SKMUS2001014, SKMUS2001129, SKMUS2001157, SKMUS2001164, SKMUS2001199, SKMUS2001201, SKMUS2001215, SKMUS2001247, SKMUS2001254, SKMUS2001271, SKMUS2001287, SKMUS2001364, SKMUS2001398, SKMUS2001402, SKMUS2001412, SKMUS2001492, SKMUS2001501, SKMUS2001543, SKMUS2001580, SKMUS2001585,

SKMUS2001608, SKMUS2001631, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003744, SKMUS2004047, SKMUS2004483, SKMUS2004667, SKMUS2004897, SKMUS2004903, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232,

SKMUS2009479, SKMUS2009557, SKNMC1000110, SKNMC1000123, SKNMC1000124, SKNMC1000229, SKNMC1000251, SKNMC1000264, SKNMC2000224, SKNMC2000256, SKNMC2000356, SKNMC2000374, SKNMC2000583, SKNMC2000593, SKNMC2000612, SKNMC2000622, SKNMC2000698, SKNMC2000966, SKNMC2001113, SKNMC2001503, SKNMC2001555, SKNMC2001596, SKNMC2002402, SKNMC2003639, SKNMC2003924, SKNMC2003987, SKNMC2004457, SKNMC2004643, SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000002, SKNSH1000086, SKNSH1000174, SKNSH1000301, SKNSH1000308, SKNSH1000416, SKNSH2000051, SKNSH2000101, SKNSH2000163, SKNSH2000250, SKNSH2000319, SKNSH2000482,

SKNSH2000516, SKNSH2000550, SKNSH2000716, SKNSH2000971, SKNSH2001222, SKNSH2001875, SKNSH2001931, SKNSH2002054, SKNSH2002325, SKNSH2002768, SKNSH2002866, SKNSH2003064, SKNSH2003174, SKNSH2003422, SKNSH2003466, SKNSH2003483, SKNSH2003528, SKNSH2004039, SKNSH2005061, SKNSH2005120, SKNSH2005194, SKNSH2005792, SKNSH2005816, SKNSH2005981, SKNSH2006234, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009357, SKNSH2009435, SKNSH2009991, SKNSH2010015, SMINT1000012, SMINT1000039, SMINT1000042, SMINT1000048, SMINT1000051, SMINT1000054, SMINT1000071, SMINT1000075, SMINT1000078, SMINT1000091,

SMINT1000100, SMINT1000103, SMINT1000118, SMINT1000131, SMINT1000137, SMINT1000192, SMINT2000007, SMINT2000032, SMINT2000040, SMINT2000060, SMINT2000073, SMINT2000084, SMINT2000145, SMINT2000159, SMINT2000176, SMINT2000185, SMINT2000227, SMINT2000229, SMINT2000232, SMINT2000239, SMINT2000267, SMINT2000277, SMINT2000396, SMINT2000400, SMINT2000468, SMINT2000518, SMINT2000529, SMINT2000530, SMINT2000537, SMINT2000538, SMINT2000541, SMINT2000558, SMINT2000562, SMINT2000568, SMINT2000602, SMINT2000609, SMINT2000747, SMINT2000811, SMINT2000824, SMINT2000984, SMINT2001114, SMINT2001158, SMINT2001183, SMINT2001199, SMINT2001222, SMINT2001348, SMINT2001397, SMINT2001458, SMINT2001461, SMINT2001535,

SMINT2001559, SMINT2001609, SMINT2001615, SMINT2001683, SMINT2001731, SMINT2001950, SMINT2002126, SMINT2002159, SMINT2002202, SMINT2002210, SMINT2002224, SMINT2002281, SMINT2002311, SMINT2002328, SMINT2002414, SMINT2002457, SMINT2002620, SMINT2002743, SMINT2002778, SMINT2002880, SMINT2002884, SMINT2002899, SMINT2002974, SMINT2002976, SMINT2003003, SMINT2003128, SMINT2003150, SMINT2003317, SMINT2003340, SMINT2003386, SMINT2003423, SMINT2003505, SMINT2003551, SMINT2003569, SMINT2003641, SMINT2003644, SMINT2003866, SMINT2004023, SMINT2004037, SMINT2004047, SMINT2004086, SMINT2004144, SMINT2004280, SMINT2004339, SMINT2004350, SMINT2004414, SMINT2004473, SMINT2004547, SMINT2004583, SMINT2004729,

SMINT2004781, SMINT2004891, SMINT2004909, SMINT2004972, SMINT2004992, SMINT2005075, SMINT2005161, SMINT2005174, SMINT2005213, SMINT2005330, SMINT2005387, SMINT2005402, SMINT2005405, SMINT2005621, SMINT2005623, SMINT2005624, SMINT2005688, SMINT2005800, SMINT2005956, SMINT2006078, SMINT2006205, SMINT2006331, SMINT2006596, SMINT2006641, SMINT2006648, SMINT2006708, SMINT2006716, SMINT2007062, SMINT2007101, SMINT2007140, SMINT2007187, SMINT2007219, SMINT2007294, SMINT2007365, SMINT2007391, SMINT2007433, SMINT2007508, SMINT2007526, SMINT2007579, SMINT2007647, SMINT2007790, SMINT2007792, SMINT2007796, SMINT2007867, SMINT2007932, SMINT2008042, SMINT2008054, SMINT2008133, SMINT2008277, SMINT2008329,

SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2009119, SMINT2009121, SMINT2009212, SMINT2009216, SMINT2009233, SMINT2009259, SMINT2009272, SMINT2009363, SMINT2009415, SMINT2009468, SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010426, SMINT2010500, SMINT2010533, SMINT2010629, SMINT2010672, SMINT2010753, SMINT2010853, SMINT2010897, SMINT2010959,

SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012179, SMINT2012195, SMINT2012285, SMINT2012291, SMINT2012501, SMINT2012734, SMINT2012793, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014489, SMINT2014721, SMINT2015006, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016440, SMINT2016477,

SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000041, SPLEN1000049, SPLEN1000056, SPLEN1000087, SPLEN1000089, SPLEN1000091, SPLEN1000134, SPLEN1000139, SPLEN1000143, SPLEN1000144, SPLEN1000145, SPLEN1000156, SPLEN1000163, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189,

SPLEN2000197, SPLEN2000205, SPLEN2000210, SPLEN2000222, SPLEN2000242, SPLEN2000243, SPLEN2000247, SPLEN2000258, SPLEN2000270, SPLEN2000307, SPLEN2000310, SPLEN2000341, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000537, SPLEN2000541, SPLEN2000607, SPLEN2000630, SPLEN2000695, SPLEN2000698, SPLEN2001135, SPLEN2001157, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001626, SPLEN2001650, SPLEN2001689, SPLEN2001710, SPLEN2001781, SPLEN2001881, SPLEN2001912, SPLEN2001945,

SPLEN2002007, SPLEN2002053, SPLEN2002147, SPLEN2002166, SPLEN2002175, SPLEN2002223, SPLEN2002272, SPLEN2002314, SPLEN2002343, SPLEN2002354, SPLEN2002385, SPLEN2002419, SPLEN2002451, SPLEN2002462, SPLEN2002463, SPLEN2002467, SPLEN2002477, SPLEN2002493, SPLEN2002662, SPLEN2002695, SPLEN2002707, SPLEN2002744, SPLEN2002917, SPLEN2002931, SPLEN2002957, SPLEN2003160, SPLEN2003204, SPLEN2003219, SPLEN2003297, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004343, SPLEN2004346, SPLEN2004368, SPLEN2004611, SPLEN2004662, SPLEN2004844, SPLEN2005009, SPLEN2005227, SPLEN2005272, SPLEN2005342, SPLEN2005416,

SPLEN2005450, SPLEN2005474, SPLEN2005560, SPLEN2005727, SPLEN2005783, SPLEN2005790, SPLEN2005806, SPLEN2005838, SPLEN2005927, SPLEN2005939, SPLEN2006143, SPLEN2006268, SPLEN2006283, SPLEN2006325, SPLEN2006389, SPLEN2006425, SPLEN2006671, SPLEN2006701, SPLEN2006785, SPLEN2006875, SPLEN2007182, SPLEN2007276, SPLEN2007314, SPLEN2007326, SPLEN2007350, SPLEN2007388, SPLEN2007619, SPLEN2007653, SPLEN2007689, SPLEN2007695, SPLEN2007794, SPLEN2007879, SPLEN2007926, SPLEN2007951, SPLEN2008007, SPLEN2008135, SPLEN2008164, SPLEN2008317, SPLEN2008496, SPLEN2008786, SPLEN2009088, SPLEN2009163, SPLEN2009315, SPLEN2009340, SPLEN2009512, SPLEN2009513, SPLEN2009541, SPLEN2009548, SPLEN2009581, SPLEN2009733,

SPLEN2009803, SPLEN2009884, SPLEN2009918, SPLEN2010004, SPLEN2010015, SPLEN2010112, SPLEN2010119, SPLEN2010146, SPLEN2010187, SPLEN2010350, SPLEN2010395, SPLEN2010415, SPLEN2010447, SPLEN2010469, SPLEN2010510, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011678, SPLEN2011737, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453,

SPLEN2012523, SPLEN2012524, SPLEN2012525, SPLEN2012606, SPLEN2012611, SPLEN2012619, SPLEN2012624, SPLEN2012725, SPLEN2012743, SPLEN2012756, SPLEN2012846, SPLEN2012889, SPLEN2012961, SPLEN2013108, SPLEN2013195, SPLEN2013503, SPLEN2013644, SPLEN2013648, SPLEN2013670, SPLEN2013673, SPLEN2013690, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014645, SPLEN2014669, SPLEN2014711, SPLEN2014739, SPLEN2014860, SPLEN2014865, SPLEN2014919,

SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016304, SPLEN2016356, SPLEN2016480, SPLEN2016531, SPLEN2016533, SPLEN2016541, SPLEN2016552, SPLEN2016602, SPLEN2016627, SPLEN2016678, SPLEN2016743, SPLEN2016773, SPLEN2016781, SPLEN2016840, SPLEN2016863,

SPLEN2016922, SPLEN2016932, SPLEN2016973, SPLEN2017104, SPLEN2017147, SPLEN2017187, SPLEN2017189, SPLEN2017212, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017918, SPLEN2017999, SPLEN2018098, SPLEN2018112, SPLEN2018157, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019257, SPLEN2019311, SPLEN2019323, SPLEN2019349, SPLEN2019375, SPLEN2019379, SPLEN2019446,

SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021231, SPLEN2021273, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022591, SPLEN2022785, SPLEN2022920,

SPLEN2023024, SPLEN2023118, SPLEN2023423, SPLEN2023710, SPLEN2023791, SPLEN2023977, SPLEN2024070, SPLEN2024127, SPLEN2024232, SPLEN2024273, SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028994, SPLEN2029051, SPLEN2029176, SPLEN2029295, SPLEN2029380, SPLEN2029522,

SPLEN2029683, SPLEN2029727, SPLEN2029904, SPLEN2029912, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036103, SPLEN2036326, SPLEN2036475,

SPLEN2036501, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038407, SPLEN2038473, SPLEN2039311, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041585, SPLEN2041645, SPLEN2041700, SPLEN2041720, SPLEN2041977, SPLEN2042051, SPLEN2042303, SPLEN2042521, SPLEN2042598, SPLEN2042714, SPLEN2042920, STOMA1000013, STOMA1000047, STOMA1000052, STOMA1000074, STOMA1000091, STOMA1000117, STOMA1000133,

STOMA1000175, STOMA1000186, STOMA1000190, STOMA1000191, STOMA2000032, STOMA2000072, STOMA2000088, STOMA2000104, STOMA2000148, STOMA2000168, STOMA2000183, STOMA2000188, STOMA2000279, STOMA2000383, STOMA2000386, STOMA2000478, STOMA2000482, STOMA2000529, STOMA2000539, STOMA2000567, STOMA2000640, STOMA2000678, STOMA2000686, STOMA2000888, STOMA2001025, STOMA2002141, STOMA2002367, STOMA2002688, STOMA2003158, STOMA2003289, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004925, STOMA2005120, STOMA2005664, STOMA2005667, STOMA2005746, STOMA2006181, STOMA2006213, STOMA2006325,

STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008838, STOMA2009250, STOMA2009253, STOMA2009256, STOMA2009289, THYMU1000010, THYMU1000017, THYMU1000025, THYMU1000029, THYMU1000032, THYMU1000033, THYMU1000041, THYMU1000060, THYMU1000074, THYMU1000098, THYMU1000103, THYMU1000105, THYMU1000136, THYMU1000142, THYMU1000144, THYMU1000158, THYMU1000176, THYMU1000179, THYMU1000191, THYMU1000198, THYMU1000217, THYMU1000240, THYMU1000242, THYMU1000291, THYMU1000316, THYMU1000318, THYMU1000336, THYMU1000366, THYMU1000382, THYMU1000393, THYMU1000394, THYMU1000399, THYMU1000426,

THYMU1000440, THYMU1000459, THYMU1000473, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140, THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000440, THYMU2000489, THYMU2000492, THYMU2000519, THYMU2000570, THYMU2000602, THYMU2000660, THYMU2000671, THYMU2000672, THYMU2000684, THYMU2000702, THYMU2000767, THYMU2000775, THYMU2000800, THYMU2000879,

THYMU2000932, THYMU2000950, THYMU2000971, THYMU2000982, THYMU2000990, THYMU2001018, THYMU2001053, THYMU2001071, THYMU2001139, THYMU2001195, THYMU2001202, THYMU2001203, THYMU2001256, THYMU2001270, THYMU2001291, THYMU2001314, THYMU2001325, THYMU2001357, THYMU2001422, THYMU2001443, THYMU2001515, THYMU2001521, THYMU2001556, THYMU2001622, THYMU2001727, THYMU2001819, THYMU2001825, THYMU2001868, THYMU2001898, THYMU2001900, THYMU2001916, THYMU2001989, THYMU2002023, THYMU2002037, THYMU2002080, THYMU2002109, THYMU2002147, THYMU2002154, THYMU2002157, THYMU2002177, THYMU2002203, THYMU2002229, THYMU2002242, THYMU2002338, THYMU2002356, THYMU2002376, THYMU2002384, THYMU2002450, THYMU2002490, THYMU2002548,

THYMU2002695, THYMU2002745, THYMU2002756, THYMU2002789, THYMU2002792, THYMU2002815, THYMU2002852, THYMU2002910, THYMU2002950, THYMU2002983, THYMU2003069, THYMU2003107, THYMU2003124, THYMU2003133, THYMU2003166, THYMU2003232, THYMU2003242, THYMU2003249, THYMU2003270, THYMU2003282, THYMU2003287, THYMU2003336, THYMU2003366, THYMU2003397, THYMU2003440, THYMU2003470, THYMU2003479, THYMU2003542, THYMU2003632, THYMU2003650, THYMU2003700, THYMU2003760, THYMU2003803, THYMU2003855, THYMU2003907, THYMU2004003, THYMU2004008, THYMU2004042, THYMU2004108, THYMU2004111, THYMU2004123, THYMU2004139, THYMU2004169, THYMU2004284, THYMU2004320, THYMU2004336, THYMU2004356, THYMU2004452, THYMU2004508, THYMU2004512,

THYMU2004635, THYMU2004639, THYMU2004668, THYMU2004677, THYMU2004688, THYMU2004693, THYMU2004733, THYMU2004835, THYMU2004843, THYMU2004906, THYMU2004916, THYMU2004986, THYMU2005001, THYMU2005003, THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005480, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005997, THYMU2006001, THYMU2006048, THYMU2006098, THYMU2006160, THYMU2006170, THYMU2006252, THYMU2006277, THYMU2006303, THYMU2006324, THYMU2006350,

THYMU2006357, THYMU2006365, THYMU2006420, THYMU2006468, THYMU2006505, THYMU2006610, THYMU2006666, THYMU2006712, THYMU2006913, THYMU2006946, THYMU2006965, THYMU2007025, THYMU2007036, THYMU2007095, THYMU2007179, THYMU2007258, THYMU2007307, THYMU2007308, THYMU2007339, THYMU2007350, THYMU2007384, THYMU2007493, THYMU2007521, THYMU2007528, THYMU2007530, THYMU2007532, THYMU2007561, THYMU2007571, THYMU2007635, THYMU2007658, THYMU2007667, THYMU2007694, THYMU2007725, THYMU2007729, THYMU2007802, THYMU2007824, THYMU2007854, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226,

THYMU2008282, THYMU2008339, THYMU2008350, THYMU2008351, THYMU2008452, THYMU2008643, THYMU2008686, THYMU2008691, THYMU2008714, THYMU2008725, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010161, THYMU2010192, THYMU2010519, THYMU2010637, THYMU2010671, THYMU2010699, THYMU2010705,

THYMU2010779, THYMU2010816, THYMU2010831, THYMU2010848, THYMU2011053, THYMU2011063, THYMU2011072, THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012073, THYMU2012104, THYMU2012113, THYMU2012273, THYMU2012401, THYMU2012492, THYMU2012507, THYMU2012565, THYMU2012574, THYMU2012631, THYMU2012665, THYMU2012690, THYMU2012701, THYMU2012807,

THYMU2012882, THYMU2012891, THYMU2012902, THYMU2012919, THYMU2013023, THYMU2013047, THYMU2013051, THYMU2013089, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015321, THYMU2015439, THYMU2015441, THYMU2015479, THYMU2015762, THYMU2015775, THYMU2015825, THYMU2015918, THYMU2015943, THYMU2015984,

THYMU2016113, THYMU2016164, THYMU2016204, THYMU2016219, THYMU2016360, THYMU2016496, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017675, THYMU2017707, THYMU2017844, THYMU2017878, THYMU2018028, THYMU2018071, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019021, THYMU2019054, THYMU2019197, THYMU2019210, THYMU2019364, THYMU2019436, THYMU2019442, THYMU2019587,

THYMU2019599, THYMU2019686, THYMU2019813, THYMU2020198, THYMU2020289, THYMU2020416, THYMU2020491, THYMU2020499, THYMU2020560, THYMU2020667, THYMU2020830, THYMU2020883, THYMU2020959, THYMU2021361, THYMU2021509, THYMU2021597, THYMU2021684, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024071, THYMU2024121, THYMU2024185, THYMU2024207, THYMU2024616, THYMU2024684, THYMU2024748, THYMU2025042, THYMU2025189, THYMU2025319, THYMU2025325, THYMU2025406, THYMU2025557, THYMU2025572, THYMU2025707, THYMU2025849, THYMU2025909, THYMU2025939, THYMU2026182, THYMU2026350,

THYMU2026530, THYMU2026531, THYMU2026835, THYMU2026904, THYMU2027053, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031579, THYMU2031847, THYMU2031890, THYMU2031994, THYMU2032014, THYMU2032035,

THYMU2032080, THYMU2032150, THYMU2032358, THYMU2032437, THYMU2032601, THYMU2032655, THYMU2032715, THYMU2032732, THYMU2032825, THYMU2032976, THYMU2033053, THYMU2033059, THYMU2033070, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2034917, THYMU2035064, THYMU2035078, THYMU2035101, THYMU2035319, THYMU2035321, THYMU2035388, THYMU2035400, THYMU2035554, THYMU2035710, THYMU2036105, THYMU2036207, THYMU2036215, THYMU2036252, THYMU2036265, THYMU2036366, THYMU2036459, THYMU2036461, THYMU2036604, THYMU2036653,

THYMU2037081, THYMU2037208, THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360,

THYMU3000390, THYMU3000420, THYMU3000490, THYMU3000504, THYMU3000542, THYMU3000655, THYMU3000826, THYMU3000841, THYMU3001077, THYMU3001082, THYMU3001083, THYMU3001133, THYMU3001151, THYMU3001379, THYMU3001472, THYMU3001593, THYMU3001883, THYMU3001939, THYMU3001991, THYMU3002452, THYMU3002578, THYMU3002661, THYMU3003007, THYMU3003092, THYMU3003212, THYMU3003350, THYMU3003403, THYMU3003565, THYMU3003763, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371,

THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008935, THYMU3009006, THYMU3009255, THYMU3009391, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701, THYMU3015042, THYMU3015457, THYMU3015571,

THYMU3015592, THYMU3015625, THYMU3015647, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642, THYMU3025683, THYMU3025772, THYMU3025865, THYMU3026000,

THYMU3026350, THYMU3026479, THYMU3026532, THYMU3026783, THYMU3026869, THYMU3027251, THYMU3027540, THYMU3027655, THYMU3027671, THYMU3028075, THYMU3028410, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032867, THYMU3033402, THYMU3033630, THYMU3033649, THYMU3033754, THYMU3033759, THYMU3034078, THYMU3034099, THYMU3034453, THYMU3034616, THYMU3034853, THYMU3034867, THYMU3034983, THYMU3036200, THYMU3036310, THYMU3036934, THYMU3036953, THYMU3037052, THYMU3037192,

THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482, THYMU3043597, THYMU3043688, THYMU3043779,

THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000066, TKIDN1000164, TKIDN1000171, TKIDN1000192, TKIDN2000127, TKIDN2000240, TKIDN2000246, TKIDN2000319, TKIDN2000464, TKIDN2000521, TKIDN2001529, TKIDN2001662, TKIDN2002318, TKIDN2002329, TKIDN2002424, TKIDN2002632, TKIDN2002738, TKIDN2003044, TKIDN2003059, TKIDN2003062, TKIDN2003078, TKIDN2003366, TKIDN2003396, TKIDN2003413, TKIDN2003597, TKIDN2003601,

TKIDN2004185, TKIDN2004386, TKIDN2004458, TKIDN2004748, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161, TKIDN2015263, TKIDN2015285, TKIDN2015788, TKIDN2016309, TKIDN2016399, TKIDN2016846,

TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002046, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192, TLIVE2007528, TLIVE2007607, TLIVE2007736, TLIVE2007816, TLIVE2007919, TLIVE2008083, TLIVE2008213, TLIVE2008221,

TLIVE2008712, TLIVE2008797, TLIVE2009087, TLIVE2009163, TLIVE2009489, TLIVE2009541, TLUNG2000179, TOVAR2000476, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000057, TRACH1000074, TRACH1000100, TRACH1000106, TRACH1000125, TRACH1000140, TRACH1000144, TRACH1000181, TRACH1000212, TRACH1000240, TRACH2000002, TRACH2000015, TRACH2000024, TRACH2000032, TRACH2000045, TRACH2000047, TRACH2000081, TRACH2000083, TRACH2000148, TRACH2000185, TRACH2000196, TRACH2000235, TRACH2000243, TRACH2000250, TRACH2000259, TRACH2000272, TRACH2000282, TRACH2000287, TRACH2000289, TRACH2000312,

TRACH2000336, TRACH2000393, TRACH2000411, TRACH2000472, TRACH2000496, TRACH2000497, TRACH2000502, TRACH2000522, TRACH2000559, TRACH2000660, TRACH2000665, TRACH2000675, TRACH2000677, TRACH2000703, TRACH2000767, TRACH2000775, TRACH2000807, TRACH2000873, TRACH2000894, TRACH2000898, TRACH2000923, TRACH2000944, TRACH2000981, TRACH2000986, TRACH2001037, TRACH2001060, TRACH2001085, TRACH2001109, TRACH2001121, TRACH2001147, TRACH2001154, TRACH2001188, TRACH2001275, TRACH2001335, TRACH2001339, TRACH2001341, TRACH2001347, TRACH2001395, TRACH2001400, TRACH2001403, TRACH2001416, TRACH2001428, TRACH2001432, TRACH2001443, TRACH2001463, TRACH2001474, TRACH2001523, TRACH2001525, TRACH2001532, TRACH2001549,

TRACH2001592, TRACH2001596, TRACH2001621, TRACH2001646, TRACH2001651, TRACH2001684, TRACH2001898, TRACH2001933, TRACH2001968, TRACH2001996, TRACH2002043, TRACH2002054, TRACH2002092, TRACH2002100, TRACH2002138, TRACH2002333, TRACH2002784, TRACH2002840, TRACH2002847, TRACH2002853, TRACH2002862, TRACH2002954, TRACH2002960, TRACH2002988, TRACH2003070, TRACH2003272, TRACH2003347, TRACH2003484, TRACH2003516, TRACH2003605, TRACH2003736, TRACH2003759, TRACH2003941, TRACH2004054, TRACH2004087, TRACH2004109, TRACH2004170, TRACH2004183, TRACH2004251, TRACH2004292, TRACH2004399, TRACH2004428, TRACH2004458, TRACH2004499, TRACH2004521, TRACH2004570, TRACH2004845, TRACH2004887, TRACH2004950, TRACH2005004,

TRACH2005017, TRACH2005066, TRACH2005159, TRACH2005314, TRACH2005444, TRACH2005666, TRACH2005698, TRACH2005710, TRACH2005720, TRACH2005769, TRACH2005796, TRACH2006015, TRACH2006049, TRACH2006097, TRACH2006194, TRACH2006327, TRACH2006331, TRACH2006387, TRACH2006528, TRACH2006670, TRACH2006762, TRACH2006870, TRACH2006918, TRACH2007059, TRACH2007324, TRACH2007358, TRACH2007483, TRACH2007542, TRACH2007577, TRACH2007674, TRACH2007676, TRACH2007688, TRACH2007733, TRACH2007754, TRACH2007816, TRACH2007894, TRACH2007988, TRACH2008112, TRACH2008113, TRACH2008127, TRACH2008130, TRACH2008278, TRACH2008478, TRACH2008540, TRACH2008583, TRACH2008597, TRACH2008820, TRACH2008891, TRACH2008921, TRACH2009006,

TRACH2009060, TRACH2009123, TRACH2009268, TRACH2009291, TRACH2009348, TRACH2009358, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587, TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011290, TRACH2011293, TRACH2011302, TRACH2011500, TRACH2011574, TRACH2011894, TRACH2011956, TRACH2012000, TRACH2012138, TRACH2012242, TRACH2012370, TRACH2012497, TRACH2012536, TRACH2012562, TRACH2012742, TRACH2012823, TRACH2012918,

TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045, TRACH2014046, TRACH2014082, TRACH2014268, TRACH2014284, TRACH2014337, TRACH2014356, TRACH2014371, TRACH2014442, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2014997, TRACH2015180, TRACH2015243, TRACH2015316, TRACH2015381, TRACH2015486, TRACH2015654, TRACH2015939, TRACH2015987, TRACH2016080, TRACH2016131, TRACH2016286, TRACH2016317, TRACH2016347,

TRACH2016380, TRACH2016384, TRACH2016410, TRACH2016481, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498, TRACH2017609, TRACH2017949, TRACH2017965, TRACH2017980, TRACH2018034, TRACH2018084, TRACH2018262, TRACH2018273, TRACH2018278, TRACH2018317, TRACH2018345, TRACH2018380, TRACH2018512, TRACH2018646, TRACH2018663, TRACH2018718, TRACH2018835, TRACH2018882, TRACH2018914, TRACH2018950, TRACH2019024, TRACH2019046, TRACH2019151, TRACH2019154, TRACH2019176, TRACH2019248, TRACH2019309, TRACH2019425, TRACH2019473, TRACH2019602, TRACH2019661, TRACH2019672, TRACH2019673,

TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839, TRACH2023246, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025569, TRACH2025705, TRACH2025749, TRACH2025753, TRACH2025810, TRACH2025853, TRACH2025911,

TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000692, TRACH3000780, TRACH3000892, TRACH3000926, TRACH3001119, TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003586, TRACH3003683, TRACH3003780, TRACH3003805, TRACH3004113, TRACH3004288, TRACH3004303, TRACH3004412, TRACH3004424, TRACH3004456,

TRACH3004537, TRACH3004596, TRACH3004651, TRACH3004652, TRACH3004671, TRACH3004747, TRACH3004760, TRACH3004783, TRACH3004786, TRACH3004840, TRACH3004931, TRACH3004934, TRACH3005072, TRACH3005085, TRACH3005102, TRACH3005173, TRACH3005191, TRACH3005274, TRACH3005294, TRACH3005549, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006800, TRACH3006889, TRACH3006985, TRACH3007097, TRACH3007268, TRACH3007274, TRACH3007277, TRACH3007391, TRACH3007541, TRACH3007595, TRACH3007627, TRACH3007689,

TRACH3007766, TRACH3007866, TRACH3007995, TRACH3008042, TRACH3008061, TRACH3008201, TRACH3008535, TRACH3008632, TRACH3008688, TRACH3008697, TRACH3008713, TRACH3008887, TRACH3008902, TRACH3008990, TRACH3009001, TRACH3009059, TRACH3009061, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942,

TRACH3013072, TRACH3013426, TRACH3013454, TRACH3013558, TRACH3013684, TRACH3013693, TRACH3013700, TRACH3013766, TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019347,

TRACH3019370, TRACH3019447, TRACH3019598, TRACH3019621, TRACH3019662, TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823,

TRACH3025302, TRACH3025316, TRACH3025346, TRACH3025520, TRACH3026148, TRACH3026283, TRACH3026299, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660,

TRACH3031678, TRACH3031936, TRACH3032044, TRACH3032065, TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036997, TRACH3037063, TRACH3037267, TRACH3037288, TRACH3037505, TRACH3037573, TRACH3037696,

TRACH3037897, TRACH3038086, TRACH3038399, TSTOM1000135, TSTOM1000186, TSTOM2000235, TSTOM2000442, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682, TUTER1000014, TUTER1000137, TUTER2000057, TUTER2000283, TUTER2000425, TUTER2000916, TUTER2001286, TUTER2001341, TUTER2001387, TUTER2001433, TUTER2001461, TUTER2002028, TUTER2002074, TUTER2002158, TUTER2002228, TUTER2002323, TUTER2002356, TUTER2002729, UMVEN1000122, UMVEN1000156, UMVEN1000186, UMVEN2000046, UMVEN2000069, UMVEN2000121, UMVEN2000354, UTERU1000008, UTERU1000015, UTERU1000024, UTERU1000031, UTERU1000032, UTERU1000057,

UTERU1000065, UTERU1000077, UTERU1000093, UTERU1000096, UTERU1000106, UTERU1000109, UTERU1000131, UTERU1000138, UTERU1000148, UTERU1000160, UTERU1000183, UTERU1000187, UTERU1000192, UTERU1000249, UTERU1000337, UTERU1000339, UTERU2000047, UTERU2000074, UTERU2000099, UTERU2000154, UTERU2000197, UTERU2000218, UTERU2000238, UTERU2000243, UTERU2000260, UTERU2000300, UTERU2000329, UTERU2000338, UTERU2000349, UTERU2000377, UTERU2000393, UTERU2000418, UTERU2000465, UTERU2000485, UTERU2000517, UTERU2000524, UTERU2000537, UTERU2000539, UTERU2000541, UTERU2000546, UTERU2000550, UTERU2000569, UTERU2000607, UTERU2000629, UTERU2000649, UTERU2000663, UTERU2000696, UTERU2000794, UTERU2000830, UTERU2000844,

UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002332, UTERU2002473, UTERU2002547, UTERU2002693, UTERU2002733, UTERU2002736, UTERU2002737, UTERU2002826, UTERU2002964, UTERU2003035, UTERU2003057, UTERU2003126, UTERU2003135, UTERU2003399, UTERU2003411, UTERU2003456, UTERU2003577, UTERU2003704, UTERU2003926, UTERU2003973, UTERU2004015, UTERU2004037, UTERU2004039, UTERU2004061, UTERU2004163, UTERU2004299, UTERU2004461, UTERU2004520, UTERU2004564, UTERU2004698, UTERU2004861, UTERU2004929, UTERU2005004, UTERU2005050,

UTERU2005179, UTERU2005346, UTERU2005446, UTERU2005449, UTERU2005450, UTERU2005533, UTERU2005601, UTERU2005664, UTERU2005822, UTERU2005903, UTERU2005905, UTERU2006103, UTERU2006115, UTERU2006137, UTERU2006182, UTERU2006400, UTERU2006412, UTERU2006429, UTERU2006486, UTERU2006524, UTERU2006547, UTERU2006568, UTERU2006593, UTERU2006643, UTERU2006651, UTERU2006705, UTERU2006899, UTERU2007004, UTERU2007075, UTERU2007081, UTERU2007128, UTERU2007253, UTERU2007267, UTERU2007499, UTERU2007639, UTERU2007724, UTERU2007924, UTERU2007942, UTERU2008018, UTERU2008019, UTERU2008027, UTERU2008040, UTERU2008077, UTERU2008130, UTERU2008347, UTERU2008426, UTERU2008516, UTERU2008561, UTERU2008653, UTERU2008705,

UTERU2008707, UTERU2008785, UTERU2008845, UTERU2008901, UTERU2008930, UTERU2008939, UTERU2009094, UTERU2009120, UTERU2009131, UTERU2009147, UTERU2009206, UTERU2009283, UTERU2009414, UTERU2009483, UTERU2009510, UTERU2009538, UTERU2009540, UTERU2009776, UTERU2009904, UTERU2009951, UTERU2009972, UTERU2010115, UTERU2010124, UTERU2010164, UTERU2010226, UTERU2010231, UTERU2010304, UTERU2010320, UTERU2010431, UTERU2010525, UTERU2010651, UTERU2010724, UTERU2010747, UTERU2011195, UTERU2011199, UTERU2011220, UTERU2011261, UTERU2011287, UTERU2011410, UTERU2011574, UTERU2011621, UTERU2011657, UTERU2011741, UTERU2011806, UTERU2011811, UTERU2011897, UTERU2011906, UTERU2011962, UTERU2011968, UTERU2012031,

UTERU2012101, UTERU2012114, UTERU2012252, UTERU2012333, UTERU2012407, UTERU2012526, UTERU2012581, UTERU2012610, UTERU2012615, UTERU2012688, UTERU2012703, UTERU2012715, UTERU2012741, UTERU2012767, UTERU2012786, UTERU2012856, UTERU2012890, UTERU2012938, UTERU2012976, UTERU2013048, UTERU2013078, UTERU2013231, UTERU2013280, UTERU2013322, UTERU2013483, UTERU2013491, UTERU2013502, UTERU2013586, UTERU2013926, UTERU2013976, UTERU2014001, UTERU2014223, UTERU2014398, UTERU2014464, UTERU2014548, UTERU2014631, UTERU2014668, UTERU2014678, UTERU2014728, UTERU2014998, UTERU2015062, UTERU2015087, UTERU2015108, UTERU2015190, UTERU2015198, UTERU2015202, UTERU2015405, UTERU2015640, UTERU2015830, UTERU2015880,

UTERU2016147, UTERU2016157, UTERU2016464, UTERU2016669, UTERU2016757, UTERU2016761, UTERU2016799, UTERU2016896, UTERU2016902, UTERU2016979, UTERU2016981, UTERU2017123, UTERU2017303, UTERU2017421, UTERU2017492, UTERU2017613, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018884, UTERU2018955, UTERU2019005, UTERU2019038, UTERU2019163, UTERU2019257, UTERU2019453, UTERU2019491, UTERU2019534, UTERU2019681, UTERU2019706, UTERU2019710,

UTERU2019940, UTERU2019959, UTERU2019964, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023262, UTERU2023550, UTERU2023651, UTERU2023687, UTERU2023712, UTERU2023941, UTERU2024002, UTERU2024042, UTERU2024141, UTERU2024481, UTERU2024656, UTERU2024758, UTERU2024820, UTERU2024881, UTERU2024969, UTERU2025025, UTERU2025041, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591, UTERU2027616, UTERU2027941,

UTERU2028377, UTERU2028734, UTERU2029503, UTERU2029660, UTERU2029742, UTERU2029953, UTERU2030270, UTERU2030280, UTERU2031060, UTERU2031084, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034147, UTERU2034695, UTERU2035114, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036347, UTERU2036507, UTERU2036512, UTERU2036530, UTERU2036623,

UTERU2036690, UTERU2037224, UTERU2037361, UTERU2037423, UTERU2037577, UTERU2037674, UTERU2037791, UTERU2037843, UTERU2038171, UTERU2038251, UTERU3000226, UTERU3000298, UTERU3000402, UTERU3000645, UTERU3000665, UTERU3000670, UTERU3000727, UTERU3000738, UTERU3000828, UTERU3000899, UTERU3000959, UTERU3001029, UTERU3001053, UTERU3001059, UTERU3001158, UTERU3001240, UTERU3001394, UTERU3001542, UTERU3001558, UTERU3001571, UTERU3001585, UTERU3001632, UTERU3001652, UTERU3001766, UTERU3001988, UTERU3002209, UTERU3002218, UTERU3002383, UTERU3002600, UTERU3002620, UTERU3002667, UTERU3002701, UTERU3002731, UTERU3002768, UTERU3002786, UTERU3002993, UTERU3003116, UTERU3003135, UTERU3003178, UTERU3003465,

UTERU3003495, UTERU3003523, UTERU3003774, UTERU3003776, UTERU3004384, UTERU3004418, UTERU3004477, UTERU3004523, UTERU3004616, UTERU3004635, UTERU3004709, UTERU3004938, UTERU3004992, UTERU3005049, UTERU3005205, UTERU3005230, UTERU3005264, UTERU3005422, UTERU3005460, UTERU3005536, UTERU3005585, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007021, UTERU3007104, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690,

UTERU3009775, UTERU3009979, UTERU3009987, UTERU3010029, UTERU3010526, UTERU3010604, UTERU3010892, UTERU3010919, UTERU3011063, UTERU3011092, UTERU3011273, UTERU3011398, UTERU3011558, UTERU3011579, UTERU3011795, UTERU3011837, UTERU3012293, UTERU3012414, UTERU3012476, UTERU3012599, UTERU3012999, UTERU3013167, UTERU3013302, UTERU3013781, UTERU3014274, UTERU3014446, UTERU3014611, UTERU3014647, UTERU3014791, UTERU3014906, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3018154, UTERU3018172, UTERU3018255, UTERU3018616, UTERU3018711, UTERU3019078

Of the 17,176 genes that include the cDNAs of the present invention, genes whose expression levels were high enough to detect in each of the THYMU, TKIDN, TLIVE, TLUNG, TOVAR, TRACH, and TSTOM libraries were analyzed for expression frequency. The analysis results are described below. Each is a relative value, where the expression level over all tissues is taken as 100. The larger this value, the greater the level of expression.

TABLE 39

| Clone Name | THYMU | TKIDN | TLIVE | TLUNG | TOVAR | TRACH | TSTOM |
|---|---|---|---|---|---|---|---|
| 3NB691000116 | 0 | 0 | 0 | 0 | 0 | 1.822 | 0 |
| 3NB691000173 | 0 | 0 | 5.429 | 0 | 0 | 0 | 0 |
| 3NB692000281 | 0 | 0.815 | 1.513 | 0 | 4.795 | 0.247 | 0 |
| 3NB692000330 | 0.736 | 0 | 3.032 | 0 | 0 | 0.494 | 9.473 |
| 3NB692000374 | 0 | 8.029 | 0 | 0 | 0 | 0 | 0 |
| 3NB692000545 | 2.352 | 0 | 0 | 0 | 0 | 0 | 0 |
| 3NB692000912 | 1.175 | 1.739 | 0 | 0 | 0 | 0 | 0 |
| 3NB692001022 | 0 | 0 | 8.554 | 0 | 0 | 0 | 0 |
| 3NB692001288 | 0 | 22.184 | 0 | 0 | 0 | 1.678 | 0 |
| 3NB692001334 | 0.224 | 2.981 | 0 | 0 | 0 | 0.601 | 0 |
| 3NB692001442 | 2.014 | 0 | 0 | 0 | 0 | 0 | 0 |
| 3NB692001520 | 10.259 | 0 | 0 | 0 | 0 | 0 | 0 |
| 3NB692001538 | 0 | 0 | 0 | 0 | 0 | 2.225 | 0 |
| 3NB692001853 | 0 | 0 | 0 | 0 | 0 | 0 | 56.236 |
| 3NB692002051 | 0 | 31.623 | 0 | 0 | 0 | 0 | 0 |
| ADRGL1000002 | 0 | 6.095 | 0 | 0 | 0 | 0 | 0 |
| ADRGL1000018 | 4.032 | 0 | 0 | 0 | 0 | 0 | 0 |
| ADRGL1000111 | 12.580 | 0 | 0 | 0 | 0 | 0 | 0 |
| ADRGL2000056 | 12.580 | 0 | 0 | 0 | 0 | 0 | 0 |
| ADRGL2000097 | 0 | 0 | 0 | 0 | 0 | 2.490 | 0 |
| ADRGL2002260 | 12.580 | 0 | 0 | 0 | 0 | 0 | 0 |
| ADRGL2002477 | 0 | 6.297 | 0 | 0 | 0 | 3.811 | 0 |
| ADRGL2004676 | 3.635 | 0 | 0 | 0 | 0 | 0 | 0 |
| ADRGL2007313 | 0 | 36.270 | 0 | 0 | 0 | 0 | 0 |
| ADRGL2008535 | 12.580 | 0 | 0 | 0 | 0 | 0 | 0 |
| ADRGL2009273 | 0 | 5.142 | 0 | 0 | 0 | 4.668 | 0 |
| ADRGL2011330 | 12.580 | 0 | 0 | 0 | 0 | 0 | 0 |
| ADRGL2012038 | 1.714 | 0 | 0 | 0 | 0 | 0 | 0 |
| ASTRO2000046 | 0.340 | 3.021 | 0 | 0 | 0 | 11.427 | 0 |
| ASTRO2000141 | 1.266 | 0 | 10.441 | 0 | 0 | 0 | 0 |
| ASTRO2000278 | 32.494 | 0 | 0 | 0 | 0 | 0 | 0 |
| ASTRO2000480 | 4.722 | 0 | 0 | 0 | 0 | 3.171 | 0 |
| ASTRO2000801 | 0 | 3.776 | 0 | 0 | 0 | 0 | 0 |
| ASTRO2000914 | 0 | 0 | 0 | 0 | 0 | 2.180 | 0 |
| ASTRO2001029 | 2.091 | 0 | 0 | 0 | 0 | 0 | 0 |
| ASTRO2001076 | 1.185 | 0 | 0 | 0 | 0 | 0 | 0 |
| ASTRO2001806 | 0 | 0 | 0 | 0 | 0 | 1.603 | 0 |
| ASTRO2001823 | 3.631 | 0 | 0 | 0 | 0 | 0 | 0 |
| ASTRO2002024 | 32.797 | 0 | 0 | 0 | 0 | 0 | 0 |
| ASTRO2002064 | 0 | 51.654 | 0 | 0 | 0 | 0 | 0 |
| ASTRO2002202 | 3.843 | 0 | 0 | 0 | 0 | 5.163 | 0 |
| ASTRO2003024 | 1.581 | 0 | 0 | 0 | 0 | 1.062 | 0 |
| ASTRO2003316 | 2.460 | 0 | 0 | 0 | 0 | 16.521 | 0 |
| ASTRO2003461 | 4.967 | 0 | 0 | 0 | 0 | 6.672 | 0 |
| ASTRO2003632 | 0 | 0 | 0 | 8.597 | 9.049 | 0.931 | 8.922 |
| ASTRO2003840 | 5.165 | 0 | 0 | 0 | 0 | 0 | 0 |
| ASTRO2004628 | 7.422 | 0 | 0 | 0 | 0 | 0 | 0 |
| ASTRO2004974 | 3.240 | 0 | 0 | 0 | 0 | 0 | 0 |
| ASTRO2005360 | 4.974 | 0 | 0 | 0 | 0 | 6.682 | 0 |
| ASTRO2005423 | 2.871 | 0 | 0 | 0 | 0 | 3.857 | 0 |
| ASTRO2005593 | 0 | 51.654 | 0 | 0 | 0 | 0 | 0 |
| ASTRO2005687 | 0 | 0 | 0 | 0 | 0 | 4.994 | 0 |
| ASTRO2006920 | 10.005 | 0 | 0 | 0 | 0 | 0 | 0 |
| ASTRO2007047 | 1.743 | 0 | 0 | 0 | 0 | 0 | 0 |
| ASTRO2007948 | 2.088 | 18.541 | 0 | 0 | 0 | 2.805 | 0 |
| ASTRO2008216 | 2.661 | 0 | 0 | 0 | 0 | 0 | 0 |
| ASTRO2009068 | 4.718 | 20.946 | 0 | 0 | 0 | 0 | 0 |
| ASTRO2009118 | 0 | 0 | 0 | 0 | 0 | 17.497 | 0 |
| ASTRO2010582 | 0 | 0 | 0 | 0 | 0 | 19.388 | 0 |
| ASTRO2010819 | 4.489 | 0 | 0 | 13.924 | 0 | 2.261 | 0 |
| ASTRO2012552 | 1.233 | 0 | 10.162 | 0 | 0 | 0 | 0 |
| ASTRO2013585 | 1.219 | 5.414 | 0 | 0 | 0 | 0 | 0 |
| ASTRO2013671 | 19.399 | 0 | 0 | 0 | 0 | 0 | 0 |
| ASTRO2014135 | 0 | 0 | 0 | 0 | 75.660 | 0 | 0 |
| ASTRO2014561 | 0 | 0 | 60.587 | 0 | 0 | 0 | 0 |
| ASTRO2014863 | 0 | 0 | 0 | 0 | 0 | 3.784 | 0 |
| ASTRO2015529 | 0 | 0 | 0 | 38.341 | 0 | 0 | 0 |
| ASTRO2016491 | 1.260 | 1.864 | 0 | 0 | 0 | 2.256 | 0 |
| ASTRO2018169 | 1.575 | 3.496 | 0 | 0 | 0 | 1.058 | 0 |
| ASTRO2018373 | 0 | 51.654 | 0 | 0 | 0 | 0 | 0 |
| BGGI11000123 | 0.584 | 0 | 0 | 0 | 0 | 1.570 | 0 |
| BGGI12000054 | 0.393 | 4.359 | 1.619 | 4.874 | 0 | 1.319 | 0 |
| BGGI12000533 | 0 | 0 | 0 | 0 | 0 | 2.529 | 0 |
| BGGI12000839 | 0.615 | 0 | 5.070 | 0 | 0 | 0.826 | 15.837 |
| BGGI12001002 | 0 | 0 | 12.205 | 0 | 0 | 0 | 0 |
| BGGI12001075 | 1.651 | 0 | 0 | 0 | 0 | 4.435 | 0 |
| BGGI12001682 | 0 | 3.823 | 0 | 0 | 0 | 0 | 0 |
| BLADE2000492 | 0 | 0 | 0 | 0 | 0 | 0 | 75.368 |
| BLADE2004089 | 0 | 0 | 0 | 0 | 0 | 11.100 | 0 |
| BLADE2006412 | 5.063 | 0 | 0 | 0 | 0 | 0 | 0 |
| BLADE2006830 | 0 | 6.222 | 0 | 0 | 0 | 0 | 0 |
| BLADE2007589 | 12.366 | 0 | 0 | 0 | 0 | 0 | 0 |
| BLADE2007666 | 5.682 | 0 | 0 | 0 | 0 | 0 | 0 |
| BLADE2008398 | 0 | 0 | 0 | 0 | 0 | 6.946 | 0 |
| BLADE2008461 | 6.201 | 0 | 0 | 0 | 0 | 0 | 0 |
| BNGH41000098 | 0 | 0 | 0 | 0 | 0 | 2.839 | 0 |
| BNGH41000137 | 3.618 | 0 | 0 | 0 | 0 | 0 | 0 |
| BNGH41000153 | 0 | 0 | 0 | 0 | 0 | 5.264 | 0 |
| BNGH41000190 | 4.220 | 0 | 0 | 0 | 0 | 5.669 | 0 |
| BNGH41000235 | 5.435 | 0 | 0 | 0 | 0 | 0 | 0 |
| BNGH42000360 | 0.131 | 0 | 0 | 0 | 0 | 0.176 | 0 |
| BNGH42000739 | 0.519 | 2.303 | 0 | 0 | 0 | 1.394 | 0 |
| BNGH42002168 | 0 | 0 | 0 | 0 | 0 | 1.490 | 0 |
| BNGH42005968 | 3.830 | 0 | 0 | 0 | 0 | 5.146 | 0 |
| BNGH42006135 | 2.753 | 0 | 0 | 0 | 14.381 | 2.958 | 0 |
| BNGH42007366 | 0 | 0 | 0 | 0 | 0 | 7.163 | 0 |
| BNGH42007460 | 0 | 3.730 | 0 | 0 | 0 | 0 | 0 |
| BNGH42007709 | 0.192 | 0.852 | 0 | 0 | 0 | 0.516 | 4.943 |
| BNGH42007788 | 2.567 | 0 | 0 | 0 | 0 | 3.448 | 0 |
| BNGH42008603 | 1.206 | 10.708 | 0 | 0 | 0 | 0 | 0 |
| BNGH42008743 | 0 | 0 | 0 | 0 | 0 | 1.724 | 33.061 |
| BRACE1000020 | 0 | 0 | 0 | 0 | 0 | 12.608 | 0 |
| BRACE1000042 | 1.269 | 0 | 0 | 0 | 0 | 0 | 0 |
| BRACE1000070 | 1.491 | 0 | 12.290 | 0 | 0 | 2.002 | 0 |
| BRACE1000073 | 0 | 31.214 | 0 | 0 | 0 | 0 | 0 |
| BRACE1000115 | 0.156 | 0 | 0 | 0 | 0 | 0 | 0 |
| BRACE1000131 | 2.606 | 0 | 0 | 0 | 0 | 2.333 | 0 |
| BRACE1000159 | 1.540 | 6.835 | 0 | 0 | 0 | 0 | 0 |
| BRACE1000166 | 54.019 | 0 | 0 | 0 | 0 | 0 | 0 |
| BRACE1000186 | 0 | 16.007 | 0 | 0 | 0 | 0 | 0 |
| BRACE2000061 | 32.684 | 0 | 0 | 0 | 0 | 0 | 0 |
| BRACE2000245 | 14.749 | 0 | 0 | 0 | 0 | 0 | 0 |
| BRACE2000300 | 0 | 39.353 | 0 | 0 | 0 | 0 | 0 |
| BRACE2000332 | 0 | 0 | 0 | 0 | 0 | 3.040 | 0 |
| BRACE2000347 | 2.156 | 0 | 17.773 | 0 | 0 | 5.791 | 0 |
| BRACE2000487 | 6.842 | 0 | 0 | 0 | 0 | 0 | 0 |
| BRACE2000525 | 0 | 18.043 | 0 | 0 | 0 | 1.820 | 0 |
| BRACE2000565 | 0 | 53.096 | 0 | 0 | 0 | 0 | 0 |
| BRACE2000753 | 25.397 | 0 | 0 | 0 | 0 | 0 | 0 |
| BRACE2000864 | 0 | 9.768 | 0 | 0 | 0 | 0 | 0 |
| BRACE2000893 | 0 | 16.728 | 0 | 0 | 0 | 5.062 | 0 |
| BRACE2000975 | 0 | 9.485 | 0 | 0 | 0 | 0 | 0 |
| BRACE2001065 | 0 | 24.580 | 0 | 0 | 0 | 7.438 | 0 |
| BRACE2001117 | 2.293 | 0 | 0 | 0 | 0 | 0 | 0 |
| BRACE2001143 | 0 | 5.499 | 0 | 0 | 0 | 0 | 0 |
| BRACE2001154 | 8.859 | 39.329 | 0 | 0 | 0 | 0 | 0 |
| BRACE2001188 | 0 | 0 | 0 | 0 | 61.486 | 0 | 0 |
| BRACE2001313 | 25.397 | 0 | 0 | 0 | 0 | 0 | 0 |
| BRACE2001477 | 2.868 | 0 | 0 | 0 | 0 | 3.852 | 0 |
| BRACE2001496 | 0 | 17.958 | 0 | 0 | 0 | 0 | 0 |
| BRACE2001497 | 0 | 13.795 | 0 | 0 | 0 | 2.087 | 0 |
| BRACE2001673 | 0 | 0.868 | 0 | 0 | 0 | 0 | 0 |
| BRACE2001692 | 1.010 | 2.241 | 4.163 | 0 | 0 | 0.678 | 0 |
| BRACE2001709 | 2.706 | 0 | 0 | 0 | 0 | 0 | 0 |
| BRACE2001711 | 0 | 0 | 26.743 | 0 | 0 | 0 | 0 |
| BRACE2001834 | 0 | 14.049 | 0 | 0 | 0 | 0 | 0 |
| BRACE2001855 | 1.919 | 0 | 0 | 0 | 0 | 0 | 0 |
| BRACE2001892 | 0 | 4.485 | 0 | 0 | 0 | 0 | 0 |
| BRACE2001942 | 54.019 | 0 | 0 | 0 | 0 | 0 | 0 |
| BRACE2001944 | 0 | 23.221 | 0 | 0 | 0 | 0 | 0 |
| BRACE2001971 | 0 | 0 | 0 | 0 | 0 | 2.512 | 0 |
| BRACE2002050 | 0 | 0 | 0 | 0 | 0 | 3.248 | 0 |
| BRACE2002081 | 16.001 | 0 | 0 | 0 | 0 | 0 | 0 |
| BRACE2002091 | 5.283 | 0 | 0 | 0 | 0 | 0 | 0 |
| BRACE2002392 | 0 | 6.380 | 0 | 0 | 0 | 0 | 0 |
| BRACE2002649 | 8.829 | 0 | 0 | 0 | 0 | 23.719 | 0 |
| BRACE2002661 | 2.401 | 0 | 19.799 | 0 | 0 | 0 | 0 |
| BRACE2002677 | 1.610 | 0 | 0 | 0 | 0 | 2.162 | 41.460 |
| BRACE2002685 | 0 | 12.688 | 0 | 0 | 0 | 0 | 0 |
| BRACE2002755 | 0 | 0 | 0 | 0 | 0 | 61.213 | 0 |
| BRACE2002812 | 0 | 21.753 | 0 | 0 | 0 | 0 | 0 |
| BRACE2002860 | 4.199 | 0 | 0 | 0 | 0 | 0 | 0 |
| BRACE2003037 | 0 | 7.993 | 0 | 0 | 0 | 0 | 0 |
| BRACE2003172 | 0 | 1.203 | 0 | 0 | 0 | 0.728 | 0 |
| BRACE2003254 | 0 | 0 | 0 | 0 | 0 | 1.014 | 0 |
| BRACE2003304 | 1.823 | 0 | 0 | 0 | 0 | 2.449 | 0 |
| BRACE2003527 | 4.299 | 0 | 0 | 0 | 0 | 0 | 0 |
| BRACE2003594 | 9.736 | 0 | 0 | 0 | 0 | 0 | 0 |
| BRACE2003628 | 5.689 | 0 | 0 | 0 | 0 | 0 | 0 |
| BRACE2003785 | 0 | 3.597 | 0 | 0 | 0 | 0 | 0 |
| BRACE2003788 | 32.523 | 0 | 0 | 0 | 0 | 0 | 0 |
| BRACE2003800 | 0 | 0 | 0 | 0 | 0 | 30.700 | 0 |
| BRACE2003827 | 0.731 | 3.243 | 0 | 0 | 0 | 1.963 | 0 |
| BRACE2003845 | 7.598 | 0 | 0 | 0 | 0 | 0 | 0 |
| BRACE2003846 | 1.087 | 0 | 0 | 0 | 0 | 2.920 | 0 |
| BRACE2003848 | 0 | 0 | 0 | 0 | 0 | 61.213 | 0 |
| BRACE2003892 | 1.820 | 0 | 0 | 0 | 0 | 2.445 | 0 |
| BRACE2003944 | 0 | 0 | 0 | 0 | 0 | 1.740 | 0 |
| BRACE2003982 | 0 | 45.699 | 0 | 0 | 0 | 0 | 0 |
| BRACE2005087 | 4.325 | 0 | 0 | 0 | 0 | 4.648 | 0 |
| BRACE2005099 | 0 | 14.762 | 0 | 0 | 0 | 0 | 0 |
| BRACE2005231 | 2.662 | 0 | 0 | 0 | 0 | 0 | 0 |
| BRACE2005298 | 25.397 | 0 | 0 | 0 | 0 | 0 | 0 |
| BRACE2005395 | 23.586 | 0 | 0 | 0 | 0 | 0 | 0 |
| BRACE2005408 | 15.862 | 35.208 | 0 | 0 | 0 | 10.654 | 0 |
| BRACE2005433 | 9.630 | 0 | 0 | 0 | 0 | 0 | 0 |
| BRACE2006055 | 0 | 7.405 | 0 | 0 | 0 | 0 | 0 |
| BRACE2006084 | 0 | 0 | 0 | 0 | 0 | 0 | 65.917 |
| BRACE2006102 | 70.146 | 0 | 0 | 0 | 0 | 0 | 0 |
| BRACE2006105 | 17.691 | 0 | 0 | 0 | 0 | 0 | 0 |
| BRACE2006162 | 0 | 0 | 0 | 32.021 | 0 | 0 | 0 |
| BRACE2006249 | 54.019 | 0 | 0 | 0 | 0 | 0 | 0 |
| BRACE2006258 | 0 | 0 | 0 | 16.951 | 0 | 0 | 0 |
| BRACE2006264 | 0 | 0 | 0 | 0 | 0 | 9.454 | 0 |
| BRACE2006319 | 8.770 | 0 | 0 | 0 | 0 | 0 | 0 |
| BRACE2006363 | 0 | 0 | 0 | 0 | 0 | 10.381 | 0 |
| BRACE2006378 | 0 | 0 | 0 | 0 | 0 | 61.213 | 0 |
| BRACE2006397 | 0.246 | 1.093 | 0 | 6.108 | 0 | 0.661 | 0 |
| BRACE2006514 | 1.449 | 3.216 | 0 | 0 | 0 | 1.946 | 0 |
| BRACE2006537 | 1.795 | 7.968 | 0 | 0 | 0 | 2.411 | 0 |
| BRACE2006547 | 0 | 0 | 0 | 0 | 0 | 16.759 | 0 |
| BRACE2006564 | 70.146 | 0 | 0 | 0 | 0 | 0 | 0 |
| BRACE2006753 | 0 | 22.762 | 0 | 0 | 0 | 0 | 0 |
| BRACE2006871 | 0.834 | 0 | 0 | 0 | 0 | 1.120 | 0 |
| BRACE2006900 | 2.145 | 0 | 0 | 0 | 11.207 | 0.576 | 0 |
| BRACE2006937 | 0 | 4.954 | 0 | 0 | 0 | 0 | 0 |
| BRACE2006982 | 0 | 4.802 | 0 | 0 | 0 | 0 | 0 |
| BRACE2007064 | 4.065 | 0 | 0 | 0 | 0 | 7.280 | 0 |
| BRACE2007068 | 20.883 | 0 | 0 | 0 | 0 | 0 | 0 |
| BRACE2007089 | 1.412 | 0 | 0 | 0 | 0 | 0 | 0 |
| BRACE2007109 | 0 | 7.067 | 0 | 0 | 0 | 0 | 0 |
| BRACE2007174 | 13.027 | 0 | 0 | 0 | 0 | 0 | 0 |
| BRACE2007191 | 6.300 | 2.543 | 0 | 0 | 0 | 0 | 0 |
| BRACE2007254 | 6.207 | 0 | 0 | 0 | 0 | 8.338 | 0 |
| BRACE2007518 | 0 | 0 | 0 | 0 | 0 | 2.542 | 0 |
| BRACE2007567 | 0 | 5.136 | 0 | 0 | 0 | 0 | 0 |
| BRACE2007767 | 0 | 0 | 0 | 0 | 0 | 0 | 66.860 |
| BRACE2007798 | 0 | 11.487 | 0 | 0 | 0 | 0 | 0 |
| BRACE2008097 | 4.003 | 0 | 0 | 0 | 0 | 0 | 0 |
| BRACE2008358 | 1.890 | 0 | 0 | 0 | 0 | 0 | 0 |
| BRACE2008380 | 0 | 7.232 | 0 | 0 | 0 | 0 | 0 |
| BRACE2008385 | 0 | 15.462 | 0 | 0 | 0 | 0 | 0 |
| BRACE2008399 | 0 | 20.487 | 0 | 0 | 0 | 0 | 0 |
| BRACE2008402 | 0 | 0 | 28.830 | 0 | 0 | 0 | 0 |
| BRACE2008653 | 2.639 | 1.952 | 0 | 0 | 0 | 3.545 | 0 |
| BRACE2008706 | 2.740 | 0 | 0 | 0 | 0 | 0 | 0 |
| BRACE2008968 | 0.485 | 2.152 | 0 | 0 | 0 | 0 | 0 |
| BRACE2009037 | 0.487 | 1.081 | 2.008 | 0 | 0 | 0.327 | 0 |
| BRACE2009044 | 8.366 | 0 | 0 | 0 | 0 | 0 | 0 |
| BRACE2009291 | 1.103 | 0 | 0 | 0 | 0 | 1.482 | 0 |
| BRACE2009483 | 7.327 | 0 | 0 | 0 | 0 | 0 | 0 |
| BRACE2009558 | 2.398 | 0 | 0 | 0 | 0 | 3.221 | 0 |
| BRACE2009620 | 16.001 | 0 | 0 | 0 | 0 | 0 | 0 |
| BRACE2010651 | 0 | 68.405 | 0 | 0 | 0 | 0 | 0 |
| BRACE2010669 | 10.631 | 0 | 0 | 0 | 0 | 0 | 0 |
| BRACE2010684 | 0 | 0 | 0 | 0 | 0 | 61.213 | 0 |
| BRACE2011183 | 0 | 23.795 | 0 | 0 | 0 | 0 | 0 |
| BRACE2011265 | 0 | 0 | 0 | 0 | 0 | 0 | 79.669 |
| BRACE2012317 | 0 | 0 | 0 | 0 | 0 | 2.306 | 0 |
| BRACE2012528 | 0.395 | 14.044 | 0 | 0 | 0 | 0 | 0 |
| BRACE2012814 | 2.557 | 0 | 0 | 0 | 0 | 1.718 | 0 |
| BRACE2013855 | 6.688 | 0 | 0 | 0 | 0 | 0 | 0 |
| BRACE2013911 | 6.268 | 0 | 0 | 0 | 0 | 0 | 0 |
| BRACE2014257 | 20.681 | 0 | 0 | 0 | 0 | 0 | 0 |
| BRACE2014306 | 0 | 18.017 | 0 | 0 | 0 | 0 | 0 |
| BRACE2014746 | 0 | 25.830 | 0 | 0 | 0 | 0 | 0 |
| BRACE2014850 | 6.254 | 0 | 0 | 0 | 0 | 0 | 0 |
| BRACE2015058 | 0 | 16.230 | 0 | 0 | 0 | 0 | 0 |
| BRACE2015287 | 3.830 | 8.501 | 15.788 | 0 | 0 | 0 | 0 |
| BRACE2016362 | 12.854 | 0 | 0 | 0 | 0 | 0 | 0 |
| BRACE2016981 | 0 | 83.911 | 0 | 0 | 0 | 0 | 0 |
| BRACE2017397 | 1.967 | 0 | 0 | 0 | 0 | 2.642 | 0 |
| BRACE2017574 | 4.272 | 0 | 0 | 0 | 0 | 0 | 0 |
| BRACE2017985 | 0 | 0 | 0 | 0 | 0 | 61.213 | 0 |
| BRACE2018337 | 0 | 8.817 | 0 | 0 | 0 | 0 | 0 |
| BRACE2018443 | 54.019 | 0 | 0 | 0 | 0 | 0 | 0 |
| BRACE2019519 | 0 | 0 | 0 | 0 | 0 | 20.849 | 0 |
| BRACE2019877 | 5.065 | 22.483 | 0 | 0 | 0 | 0 | 0 |
| BRACE2021014 | 4.940 | 0 | 0 | 0 | 0 | 6.636 | 0 |
| BRACE2021708 | 49.377 | 0 | 0 | 0 | 0 | 0 | 0 |
| BRACE2022638 | 1.243 | 0 | 0 | 0 | 0 | 0 | 0 |
| BRACE2023727 | 0 | 73.082 | 0 | 0 | 0 | 0 | 0 |
| BRACE2023801 | 31.304 | 0 | 0 | 0 | 0 | 42.051 | 0 |
| BRACE2024074 | 54.019 | 0 | 0 | 0 | 0 | 0 | 0 |
| BRACE2025437 | 2.546 | 11.301 | 0 | 0 | 0 | 3.420 | 65.567 |
| BRACE2026725 | 0 | 0 | 0 | 0 | 0 | 20.538 | 0 |
| BRACE2027643 | 12.010 | 0 | 0 | 0 | 0 | 0 | 0 |
| BRACE2027970 | 54.019 | 0 | 0 | 0 | 0 | 0 | 0 |
| BRACE2029356 | 54.019 | 0 | 0 | 0 | 0 | 0 | 0 |
| BRACE2029644 | 54.019 | 0 | 0 | 0 | 0 | 0 | 0 |
| BRACE2030323 | 0 | 0 | 0 | 0 | 0 | 35.088 | 0 |
| BRACE2030345 | 54.019 | 0 | 0 | 0 | 0 | 0 | 0 |
| BRACE2032477 | 4.647 | 0 | 0 | 0 | 0 | 12.484 | 0 |
| BRACE2035381 | 0 | 0 | 0 | 0 | 0 | 11.400 | 0 |
| BRACE2039624 | 0.807 | 0 | 0 | 0 | 0 | 0 | 0 |
| BRACE2041200 | 0 | 0 | 0 | 0 | 0 | 61.213 | 0 |
| BRACE2044286 | 0 | 0 | 0 | 0 | 0 | 20.910 | 0 |
| BRACE2047350 | 54.019 | 0 | 0 | 0 | 0 | 0 | 0 |
| BRACE3000973 | 22.478 | 0 | 0 | 0 | 0 | 0 | 0 |
| BRACE3001754 | 0 | 0 | 0 | 0 | 0 | 61.213 | 0 |
| BRACE3003413 | 54.019 | 0 | 0 | 0 | 0 | 0 | 0 |
| BRACE3003698 | 54.019 | 0 | 0 | 0 | 0 | 0 | 0 |
| BRACE3004862 | 0 | 0 | 0 | 0 | 0 | 61.213 | 0 |
| BRACE3005499 | 26.080 | 0 | 0 | 0 | 0 | 0 | 0 |
| BRACE3005938 | 0.918 | 4.076 | 0 | 0 | 0 | 2.467 | 0 |
| BRACE3005989 | 0 | 0 | 0 | 0 | 0 | 0.262 | 0 |
| BRACE3006553 | 0 | 0 | 0 | 0 | 0 | 14.418 | 0 |
| BRACE3007258 | 10.851 | 0 | 0 | 0 | 0 | 0 | 0 |
| BRACE3008036 | 0 | 68.309 | 0 | 0 | 0 | 0 | 0 |
| BRACE3008095 | 54.019 | 0 | 0 | 0 | 0 | 0 | 0 |
| BRACE3009416 | 0 | 0 | 0 | 0 | 0 | 31.969 | 0 |
| BRACE3010283 | 0 | 0 | 0 | 0 | 0 | 61.213 | 0 |
| BRACE3010428 | 1.909 | 0 | 0 | 0 | 0 | 0 | 0 |
| BRACE3010438 | 1.493 | 0 | 0 | 0 | 0 | 4.012 | 0 |
| BRACE3011421 | 0 | 0 | 0 | 0 | 0 | 31.645 | 0 |
| BRACE3012806 | 54.019 | 0 | 0 | 0 | 0 | 0 | 0 |
| BRACE3012930 | 0 | 0 | 0 | 0 | 0 | 61.213 | 0 |
| BRACE3017253 | 0 | 0 | 0 | 0 | 0 | 44.106 | 0 |
| BRACE3019941 | 0 | 0 | 0 | 0 | 0 | 3.273 | 0 |
| BRACE3020669 | 0 | 0 | 0 | 0 | 0 | 61.213 | 0 |
| BRACE3022312 | 0 | 0 | 0 | 0 | 0 | 38.115 | 0 |
| BRACE3025719 | 0 | 0 | 0 | 0 | 0 | 20.176 | 0 |
| BRACE3027478 | 0 | 0 | 0 | 0 | 0 | 17.928 | 0 |
| BRACE3027976 | 54.019 | 0 | 0 | 0 | 0 | 0 | 0 |
| BRACE3028895 | 37.949 | 0 | 0 | 0 | 0 | 0 | 0 |
| BRACE3032631 | 10.516 | 0 | 0 | 0 | 0 | 0 | 0 |
| BRACE3035168 | 70.146 | 0 | 0 | 0 | 0 | 0 | 0 |
| BRACE3040863 | 29.832 | 0 | 0 | 0 | 0 | 0 | 0 |
| BRACE3042326 | 0 | 0 | 0 | 0 | 0 | 39.301 | 0 |
| BRALZ2000988 | 4.328 | 19.213 | 0 | 0 | 0 | 0 | 0 |
| BRALZ2001038 | 0 | 0 | 0 | 0 | 0 | 18.205 | 0 |
| BRALZ2009482 | 0.310 | 0 | 2.555 | 7.690 | 0 | 2.914 | 0 |
| BRALZ2011796 | 4.596 | 0 | 0 | 0 | 0 | 0 | 0 |
| BRALZ2011880 | 5.085 | 0 | 0 | 0 | 0 | 0 | 0 |
| BRALZ2017844 | 0 | 50.604 | 0 | 0 | 0 | 0 | 0 |
| BRAMY2000354 | 3.437 | 0 | 0 | 0 | 0 | 0 | 0 |
| BRAMY2000388 | 0 | 45.295 | 0 | 0 | 0 | 0 | 0 |
| BRAMY2000508 | 29.346 | 0 | 0 | 0 | 0 | 0 | 0 |
| BRAMY2000562 | 1.602 | 21.340 | 0 | 0 | 0 | 0 | 0 |
| BRAMY2000585 | 2.048 | 0 | 0 | 0 | 0 | 0 | 0 |
| BRAMY2000814 | 0 | 78.604 | 0 | 0 | 0 | 0 | 0 |
| BRAMY2000893 | 0 | 0 | 0 | 0 | 0 | 1.705 | 0 |
| BRAMY2001678 | 62.337 | 0 | 0 | 0 | 0 | 0 | 0 |
| BRAMY2003008 | 0 | 0 | 0 | 0 | 0 | 29.622 | 0 |
| BRAMY2004363 | 3.409 | 0 | 0 | 0 | 0 | 6.870 | 0 |
| BRAMY2004521 | 5.929 | 0 | 0 | 0 | 0 | 2.655 | 0 |
| BRAMY2005488 | 9.502 | 0 | 0 | 0 | 0 | 0 | 0 |
| BRAMY2005576 | 0 | 78.604 | 0 | 0 | 0 | 0 | 0 |
| BRAMY2006366 | 0 | 0 | 63.335 | 0 | 0 | 0 | 0 |
| BRAMY2006375 | 59.294 | 0 | 0 | 0 | 0 | 0 | 0 |
| BRAMY2009013 | 5.079 | 0 | 0 | 0 | 0 | 0 | 0 |
| BRAMY2009023 | 11.054 | 49.073 | 0 | 0 | 0 | 0 | 0 |
| BRAMY2011105 | 0 | 11.213 | 0 | 0 | 0 | 0 | 0 |
| BRAMY2012091 | 4.951 | 0 | 0 | 0 | 0 | 0 | 0 |
| BRAMY2012536 | 0.963 | 4.273 | 0 | 0 | 0 | 0 | 0 |
| BRAMY2012581 | 0 | 0 | 0 | 0 | 0 | 25.588 | 0 |
| BRAMY2013169 | 0 | 0 | 0 | 0 | 0 | 12.737 | 0 |
| BRAMY2013590 | 18.015 | 0 | 0 | 0 | 0 | 0 | 0 |
| BRAMY2014754 | 1.168 | 5.185 | 0 | 0 | 0 | 0 | 0 |
| BRAMY2014813 | 8.044 | 0 | 0 | 0 | 0 | 0 | 0 |
| BRAMY2015211 | 0 | 0 | 0 | 0 | 0 | 14.550 | 0 |
| BRAMY2015550 | 28.262 | 0 | 0 | 0 | 0 | 0 | 0 |
| BRAMY2015782 | 0 | 0 | 0 | 0 | 85.296 | 0 | 0 |
| BRAMY2015833 | 3.844 | 0 | 0 | 0 | 0 | 0 | 0 |
| BRAMY2016009 | 13.082 | 0 | 0 | 0 | 0 | 0 | 0 |
| BRAMY2016386 | 45.282 | 0 | 0 | 0 | 0 | 0 | 0 |
| BRAMY2016706 | 0 | 0 | 34.076 | 0 | 0 | 0 | 0 |
| BRAMY2017864 | 11.521 | 0 | 0 | 0 | 0 | 0 | 0 |
| BRAMY2018122 | 0 | 0 | 87.217 | 0 | 0 | 0 | 0 |
| BRAMY2018785 | 0 | 25.173 | 0 | 0 | 0 | 0 | 0 |
| BRAMY2019554 | 1.996 | 0 | 0 | 0 | 0 | 1.788 | 0 |
| BRAMY2019600 | 6.733 | 0 | 0 | 0 | 0 | 0 | 0 |
| BRAMY2019963 | 0 | 25.206 | 0 | 0 | 0 | 7.628 | 0 |
| BRAMY2019989 | 3.216 | 3.569 | 0 | 0 | 0 | 1.080 | 0 |
| BRAMY2020412 | 8.951 | 0 | 0 | 0 | 0 | 0 | 0 |
| BRAMY2020427 | 1.481 | 2.192 | 0 | 0 | 0 | 0 | 0 |
| BRAMY2021139 | 0 | 0 | 0 | 0 | 0 | 28.667 | 0 |
| BRAMY2022160 | 10.505 | 0 | 0 | 0 | 0 | 0 | 0 |
| BRAMY2022168 | 11.280 | 0 | 0 | 0 | 0 | 15.152 | 0 |
| BRAMY2022445 | 0 | 0 | 0 | 0 | 0 | 52.645 | 0 |
| BRAMY2022493 | 4.424 | 0 | 0 | 0 | 0 | 2.972 | 0 |
| BRAMY2022786 | 0 | 9.238 | 0 | 0 | 0 | 1.398 | 0 |
| BRAMY2023406 | 0 | 0 | 45.388 | 0 | 0 | 0 | 0 |
| BRAMY2023729 | 0 | 0 | 0 | 0 | 0 | 52.645 | 0 |
| BRAMY2024390 | 29.138 | 0 | 0 | 0 | 0 | 0 | 0 |
| BRAMY2024416 | 0 | 16.750 | 0 | 0 | 0 | 0 | 0 |
| BRAMY2024827 | 1.663 | 0 | 0 | 0 | 0 | 0 | 0 |
| BRAMY2026009 | 0 | 55.048 | 0 | 0 | 0 | 0 | 0 |
| BRAMY2026685 | 8.266 | 0 | 0 | 0 | 0 | 13.881 | 0 |
| BRAMY2027114 | 0 | 63.622 | 0 | 0 | 0 | 0 | 0 |
| BRAMY2027587 | 33.403 | 0 | 0 | 0 | 0 | 0 | 0 |
| BRAMY2027739 | 1.122 | 0 | 0 | 0 | 0 | 0 | 0 |
| BRAMY2028516 | 0 | 0 | 81.346 | 0 | 0 | 0 | 0 |
| BRAMY2028682 | 0 | 78.604 | 0 | 0 | 0 | 0 | 0 |
| BRAMY2028740 | 0.776 | 0 | 0 | 0 | 0 | 5.214 | 0 |
| BRAMY2028796 | 0 | 0 | 0 | 0 | 0 | 1.665 | 0 |
| BRAMY2031516 | 0 | 0 | 0 | 0 | 0 | 16.547 | 0 |
| BRAMY2032087 | 9.088 | 0 | 0 | 0 | 0 | 8.139 | 0 |
| BRAMY2032696 | 0 | 78.604 | 0 | 0 | 0 | 0 | 0 |
| BRAMY2033316 | 45.282 | 0 | 0 | 0 | 0 | 0 | 0 |
| BRAMY2035070 | 45.282 | 0 | 0 | 0 | 0 | 0 | 0 |
| BRAMY2035719 | 12.665 | 0 | 0 | 0 | 0 | 0 | 0 |
| BRAMY2036076 | 0 | 40.038 | 0 | 0 | 0 | 0 | 0 |
| BRAMY2036459 | 1.571 | 0 | 0 | 0 | 0 | 4.222 | 0 |
| BRAMY2038846 | 0 | 0 | 0 | 0 | 0 | 52.645 | 0 |
| BRAMY2039872 | 16.531 | 0 | 0 | 0 | 0 | 0 | 0 |
| BRAMY2040095 | 1.140 | 0 | 2.350 | 0 | 0 | 0 | 0 |
| BRAMY2043069 | 0 | 78.604 | 0 | 0 | 0 | 0 | 0 |
| BRAMY2045530 | 7.866 | 0 | 0 | 0 | 0 | 0 | 0 |
| BRAMY2047420 | 0.845 | 0 | 0 | 0 | 0 | 0 | 0 |
| BRAMY3002120 | 45.282 | 0 | 0 | 0 | 0 | 0 | 0 |
| BRAMY3002458 | 0 | 0 | 0 | 0 | 0 | 13.906 | 0 |
| BRAMY3003205 | 20.062 | 0 | 0 | 0 | 0 | 0 | 0 |
| BRAMY3003310 | 45.282 | 0 | 0 | 0 | 0 | 0 | 0 |
| BRAMY3004364 | 11.329 | 0 | 0 | 0 | 0 | 0 | 0 |
| BRAMY3004377 | 0.692 | 0 | 0 | 0 | 18.065 | 1.858 | 0 |
| BRAMY3007311 | 0 | 29.082 | 0 | 0 | 0 | 0 | 0 |
| BRAMY3008044 | 0 | 0 | 0 | 0 | 0 | 2.009 | 0 |
| BRAMY3009491 | 26.018 | 0 | 0 | 0 | 0 | 0 | 0 |
| BRAMY3010008 | 0.485 | 0 | 0 | 0 | 0 | 0 | 0 |
| BRAMY3010492 | 14.407 | 0 | 0 | 0 | 0 | 0 | 0 |
| BRAMY4000089 | 16.974 | 0 | 0 | 0 | 0 | 5.700 | 0 |
| BRAMY4001234 | 45.282 | 0 | 0 | 0 | 0 | 0 | 0 |
| BRAMY4001863 | 12.491 | 0 | 0 | 0 | 0 | 0 | 0 |
| BRAMY4002628 | 17.768 | 0 | 0 | 0 | 0 | 23.868 | 0 |
| BRAWH1000001 | 2.775 | 0 | 0 | 0 | 0 | 0 | 0 |
| BRAWH1000007 | 7.493 | 0 | 0 | 0 | 0 | 0 | 0 |
| BRAWH1000037 | 0 | 6.750 | 0 | 0 | 0 | 0 | 0 |
| BRAWH1000040 | 0 | 5.094 | 0 | 0 | 0 | 0.771 | 0 |
| BRAWH1000045 | 0 | 7.411 | 0 | 0 | 0 | 0 | 0 |
| BRAWH1000083 | 3.778 | 0 | 0 | 0 | 0 | 0 | 0 |
| BRAWH1000093 | 3.461 | 0 | 0 | 0 | 0 | 0 | 0 |
| BRAWH1000127 | 0 | 11.833 | 0 | 0 | 0 | 0 | 0 |
| BRAWH1000162 | 0 | 20.267 | 0 | 0 | 0 | 0 | 0 |
| BRAWH1000174 | 0 | 10.605 | 0 | 0 | 0 | 0 | 0 |
| BRAWH2000082 | 0 | 9.939 | 0 | 0 | 0 | 0 | 0 |
| BRAWH2000417 | 0 | 18.297 | 0 | 0 | 0 | 0 | 0 |
| BRAWH2000460 | 2.545 | 0 | 0 | 0 | 0 | 0 | 0 |
| BRAWH2000488 | 0 | 0 | 0 | 0 | 0 | 11.293 | 0 |
| BRAWH2000494 | 0 | 33.652 | 0 | 0 | 0 | 0 | 0 |
| BRAWH2000588 | 0 | 0 | 0 | 0 | 0 | 1.405 | 0 |
| BRAWH2000633 | 0 | 0 | 0 | 0 | 0 | 2.970 | 0 |
| BRAWH2000636 | 0 | 7.824 | 0 | 0 | 0 | 6.511 | 0 |
| BRAWH2000686 | 0 | 9.050 | 0 | 0 | 0 | 0 | 0 |
| BRAWH2000697 | 0 | 0 | 0 | 0 | 0 | 2.266 | 0 |
| BRAWH2000839 | 2.055 | 0 | 2.824 | 0 | 0 | 1.380 | 0 |
| BRAWH2000859 | 0 | 0 | 0 | 0 | 0 | 52.833 | 0 |
| BRAWH2000924 | 0.239 | 7.436 | 0 | 0 | 0 | 0 | 0 |
| BRAWH2000944 | 4.350 | 0 | 0 | 0 | 0 | 0 | 0 |
| BRAWH2001019 | 1.902 | 8.442 | 0 | 0 | 0 | 0 | 0 |
| BRAWH2001092 | 0.879 | 1.301 | 3.625 | 0 | 0 | 0.394 | 0 |
| BRAWH2001129 | 0 | 0 | 0 | 0 | 0 | 52.833 | 0 |
| BRAWH2001171 | 0.515 | 0 | 0 | 0 | 0 | 0.692 | 0 |
| BRAWH2001186 | 2.376 | 0 | 0 | 0 | 0 | 0 | 0 |
| BRAWH2001239 | 6.084 | 0 | 0 | 0 | 0 | 0 | 0 |
| BRAWH2001321 | 0 | 12.711 | 0 | 0 | 0 | 0 | 0 |
| BRAWH2001395 | 0 | 9.335 | 0 | 0 | 0 | 0 | 0 |
| BRAWH2001406 | 5.646 | 0 | 0 | 0 | 0 | 0 | 72.702 |
| BRAWH2001412 | 12.538 | 0 | 0 | 0 | 0 | 0 | 0 |
| BRAWH2001418 | 0 | 9.547 | 0 | 0 | 0 | 0 | 0 |
| BRAWH2001424 | 0 | 0 | 0 | 0 | 0 | 5.703 | 0 |
| BRAWH2001439 | 4.290 | 7.617 | 0 | 0 | 0 | 1.153 | 0 |
| BRAWH2001459 | 0 | 18.107 | 0 | 0 | 0 | 0 | 0 |
| BRAWH2001461 | 0 | 0 | 0 | 73.560 | 0 | 0 | 0 |
| BRAWH2001530 | 0 | 0 | 0 | 0 | 0 | 8.550 | 0 |
| BRAWH12001535 | 0 | 12.794 | 0 | 0 | 0 | 0 | 0 |
| BRAWH2001671 | 0 | 14.075 | 0 | 0 | 0 | 0 | 0 |
| BRAWH2002062 | 45.470 | 0 | 0 | 0 | 0 | 0 | 0 |
| BRAWH2002601 | 4.147 | 0 | 0 | 0 | 0 | 5.571 | 0 |
| BRAWH2003240 | 0 | 29.050 | 0 | 0 | 0 | 0 | 0 |
| BRAWH2003689 | 0 | 0 | 0 | 0 | 0 | 52.833 | 0 |
| BRAWH2003693 | 5.706 | 12.664 | 0 | 0 | 0 | 3.832 | 0 |
| BRAWH2003818 | 2.524 | 0 | 0 | 0 | 0 | 0 | 0 |
| BRAWH2004095 | 2.957 | 0 | 0 | 0 | 0 | 0 | 0 |
| BRAWH2004136 | 13.267 | 0 | 0 | 0 | 0 | 0 | 0 |
| BRAWH2004217 | 45.470 | 0 | 0 | 0 | 0 | 0 | 0 |
| BRAWH2004842 | 0 | 14.755 | 0 | 0 | 0 | 0 | 0 |
| BRAWH2005008 | 13.767 | 0 | 0 | 0 | 0 | 0 | 0 |
| BRAWH2005068 | 0.335 | 0 | 2.765 | 0 | 8.761 | 0 | 0 |
| BRAWH2005225 | 19.334 | 0 | 0 | 0 | 0 | 0 | 0 |
| BRAWH2005578 | 0 | 0 | 0 | 0 | 0 | 15.011 | 0 |
| BRAWH2005661 | 5.332 | 5.918 | 0 | 0 | 0 | 0 | 0 |
| BRAWH2006450 | 4.187 | 18.587 | 0 | 0 | 0 | 0 | 0 |
| BRAWH2006493 | 4.241 | 0 | 0 | 0 | 0 | 0 | 0 |
| BRAWH2006526 | 45.470 | 0 | 0 | 0 | 0 | 0 | 0 |
| BRAWH2006543 | 0 | 16.237 | 0 | 0 | 0 | 4.913 | 0 |
| BRAWH2007605 | 8.552 | 0 | 0 | 0 | 0 | 2.298 | 0 |
| BRAWH2007808 | 62.515 | 0 | 0 | 0 | 0 | 0 | 0 |
| BRAWH2008058 | 19.533 | 0 | 0 | 0 | 0 | 0 | 0 |
| BRAWH2008540 | 45.470 | 0 | 0 | 0 | 0 | 0 | 0 |
| BRAWH2008706 | 0 | 0 | 0 | 0 | 0 | 6.442 | 0 |
| BRAWH2008956 | 0 | 0 | 0 | 0 | 0 | 17.441 | 0 |
| BRAWH2009238 | 13.041 | 0 | 0 | 0 | 0 | 0 | 0 |
| BRAWH2009304 | 13.082 | 0 | 0 | 0 | 0 | 0 | 0 |
| BRAWH2009307 | 0 | 7.370 | 0 | 0 | 43.369 | 0 | 0 |
| BRAWH2009360 | 0 | 0 | 0 | 0 | 0 | 9.693 | 0 |
| BRAWH2009678 | 0 | 78.731 | 0 | 0 | 0 | 0 | 0 |
| BRAWH2010329 | 2.086 | 0 | 0 | 0 | 0 | 0 | 0 |
| BRAWH2010354 | 0 | 48.038 | 0 | 0 | 0 | 0 | 0 |
| BRAWH2011096 | 45.470 | 0 | 0 | 0 | 0 | 0 | 0 |
| BRAWH2011343 | 17.548 | 0 | 0 | 0 | 0 | 0 | 0 |
| BRAWH2013219 | 0 | 35.860 | 0 | 0 | 0 | 0 | 0 |
| BRAWH2013294 | 0 | 36.500 | 0 | 0 | 0 | 0 | 0 |
| BRAWH2013871 | 62.515 | 0 | 0 | 0 | 0 | 0 | 0 |
| BRAWH2014053 | 19.364 | 0 | 0 | 0 | 0 | 0 | 0 |
| BRAWH2014188 | 4.179 | 0 | 0 | 0 | 0 | 9.623 | 0 |
| BRAWH2014645 | 7.933 | 3.913 | 0 | 0 | 0 | 3.552 | 22.702 |
| BRAWH2016166 | 0 | 0 | 0 | 0 | 0 | 25.413 | 0 |
| BRAWH2016209 | 6.926 | 0 | 0 | 0 | 0 | 0 | 0 |
| BRAWH2016514 | 29.216 | 0 | 0 | 0 | 0 | 0 | 0 |
| BRAWH2017002 | 0 | 27.634 | 0 | 0 | 0 | 0 | 0 |
| BRAWH2017685 | 1.270 | 0 | 0 | 0 | 0 | 0.568 | 0 |
| BRAWH2018206 | 0 | 40.908 | 0 | 0 | 0 | 0 | 0 |
| BRAWH2018527 | 0 | 31.509 | 0 | 0 | 0 | 0 | 0 |
| BRAWH3000088 | 0 | 8.564 | 0 | 0 | 0 | 0 | 0 |
| BRAWH3000604 | 45.470 | 0 | 0 | 0 | 0 | 0 | 0 |
| BRAWH3001319 | 22.759 | 0 | 0 | 0 | 0 | 30.573 | 0 |
| BRAWH3001918 | 0 | 0 | 0 | 0 | 0 | 15.554 | 0 |
| BRAWH3002853 | 4.317 | 0 | 0 | 0 | 0 | 0 | 0 |
| BRAWH3003411 | 38.592 | 0 | 0 | 0 | 0 | 0 | 0 |
| BRAWH3003573 | 16.812 | 0 | 0 | 0 | 0 | 0 | 0 |
| BRAWH3003727 | 0 | 0 | 0 | 0 | 0 | 33.790 | 0 |
| BRAWH3004350 | 13.825 | 0 | 0 | 0 | 0 | 0 | 0 |
| BRAWH3004530 | 0 | 0 | 0 | 0 | 0 | 19.759 | 0 |
| BRAWH3005146 | 8.444 | 0 | 0 | 0 | 0 | 11.343 | 0 |
| BRAWH3005300 | 0 | 0 | 0 | 0 | 0 | 52.833 | 0 |
| BRAWH3007726 | 45.470 | 0 | 0 | 0 | 0 | 0 | 0 |
| BRAWH3008931 | 8.664 | 0 | 0 | 0 | 0 | 0 | 0 |
| BRAWH3011637 | 0 | 0 | 0 | 0 | 0 | 69.138 | 0 |
| BRAWH3012662 | 0 | 41.917 | 0 | 0 | 0 | 0 | 0 |
| BRAWH3018063 | 0 | 0 | 0 | 0 | 0 | 2.956 | 0 |
| BRAWH3021545 | 15.210 | 0 | 0 | 0 | 0 | 8.173 | 0 |
| BRAWH3021574 | 0 | 17.397 | 0 | 0 | 0 | 0 | 0 |
| BRAWH3024242 | 0 | 0 | 0 | 0 | 0 | 35.669 | 0 |
| BRAWH3024989 | 0 | 0 | 0 | 0 | 0 | 55.728 | 0 |
| BRAWH3029806 | 0 | 0 | 0 | 0 | 0 | 9.982 | 0 |
| BRAWH3035403 | 0 | 0 | 0 | 0 | 0 | 39.582 | 0 |
| BRAWH3035936 | 29.425 | 0 | 0 | 0 | 0 | 0 | 0 |
| BRAWH3036247 | 45.470 | 0 | 0 | 0 | 0 | 0 | 0 |
| BRCAN1000076 | 2.454 | 0 | 0 | 0 | 0 | 0 | 0 |
| BRCAN2000209 | 0 | 61.737 | 0 | 0 | 0 | 0 | 0 |
| BRCAN2002173 | 0 | 14.721 | 0 | 0 | 0 | 0 | 0 |
| BRCAN2002944 | 26.657 | 0 | 0 | 0 | 0 | 0 | 0 |
| BRCAN2003944 | 0.470 | 2.085 | 0 | 0 | 0 | 0.631 | 0 |
| BRCAN2006019 | 0 | 13.906 | 0 | 0 | 0 | 0 | 0 |
| BRCAN2007409 | 5.004 | 0 | 0 | 0 | 0 | 0 | 0 |
| BRCAN2009432 | 2.196 | 0 | 0 | 0 | 0 | 0 | 0 |
| BRCAN2015719 | 0 | 0 | 0 | 0 | 0 | 3.382 | 64.842 |
| BRCAN2019653 | 5.200 | 0 | 0 | 0 | 0 | 0 | 0 |
| BRCAN2020412 | 0 | 0 | 74.979 | 0 | 0 | 0 | 0 |
| BRCAN2021024 | 8.551 | 0 | 0 | 0 | 0 | 0 | 0 |
| BRCAN2022126 | 0 | 17.973 | 0 | 0 | 0 | 0 | 0 |
| BRCAN2026197 | 0 | 11.558 | 0 | 0 | 0 | 0 | 0 |
| BRCAN2028040 | 22.049 | 0 | 0 | 0 | 0 | 0 | 0 |
| BRCAN2028319 | 21.704 | 0 | 0 | 0 | 0 | 0 | 0 |
| BRCAN2028355 | 0 | 0 | 0 | 0 | 0 | 0 | 31.728 |
| BRCOC1000040 | 9.836 | 0 | 0 | 0 | 0 | 0 | 0 |
| BRCOC1000094 | 0 | 10.574 | 0 | 0 | 0 | 0 | 0 |
| BRCOC2000004 | 0 | 6.663 | 0 | 0 | 0 | 0 | 0 |
| BRCOC2000186 | 0 | 0 | 20.450 | 0 | 64.790 | 0 | 0 |
| BRCOC2000487 | 0 | 1.072 | 0 | 0 | 0 | 0 | 0 |
| BRCOC2002155 | 0 | 0 | 0 | 0 | 0 | 24.023 | 0 |
| BRCOC2003187 | 0.656 | 0 | 0 | 0 | 0 | 0.881 | 0 |
| BRCOC2003213 | 8.657 | 0 | 0 | 0 | 0 | 0 | 0 |
| BRCOC2003732 | 0 | 0 | 0 | 0 | 0 | 24.023 | 0 |
| BRCOC2006164 | 0 | 0 | 0 | 0 | 0 | 16.280 | 0 |
| BRCOC2010123 | 0.913 | 0 | 0 | 0 | 0 | 0 | 0 |
| BRCOC2010730 | 18.026 | 0 | 0 | 0 | 0 | 0 | 0 |
| BRCOC2012172 | 0 | 0 | 0 | 0 | 0 | 28.357 | 0 |
| BRCOC2012551 | 6.014 | 0 | 0 | 0 | 0 | 24.237 | 0 |
| BRCOC2014013 | 4.522 | 20.075 | 37.284 | 0 | 0 | 0 | 0 |
| BRCOC2014033 | 13.036 | 0 | 0 | 0 | 0 | 0 | 0 |
| BRCOC2017652 | 0 | 51.098 | 0 | 0 | 0 | 0 | 0 |
| BRCOC2019903 | 13.365 | 0 | 0 | 0 | 0 | 0 | 0 |
| BRCOC2019910 | 0 | 4.627 | 0 | 0 | 0 | 0 | 0 |
| BRHIP1000104 | 0.545 | 9.678 | 0 | 0 | 0 | 0 | 0 |
| BRHIP1000108 | 0 | 15.953 | 0 | 0 | 0 | 0 | 0 |
| BRHIP2000087 | 0 | 24.363 | 0 | 0 | 0 | 0 | 0 |
| BRHIP2000239 | 0 | 0 | 0 | 0 | 0 | 52.334 | 0 |
| BRHIP2000245 | 44.974 | 0 | 0 | 0 | 0 | 0 | 0 |
| BRHIP2000534 | 0.856 | 0 | 0 | 0 | 0 | 0 | 0 |
| BRHIP2000553 | 4.285 | 0 | 0 | 0 | 0 | 0 | 0 |
| BRHIP2000819 | 12.609 | 0 | 0 | 0 | 0 | 0 | 46.392 |
| BRHIP2001099 | 17.341 | 0 | 0 | 0 | 0 | 0 | 0 |
| BRHIP2003062 | 7.921 | 0 | 0 | 0 | 0 | 0 | 0 |
| BRHIP2004359 | 40.602 | 0 | 0 | 0 | 0 | 0 | 0 |
| BRHIP2005271 | 0 | 0 | 0 | 0 | 0 | 63.472 | 0 |
| BRHIP2007305 | 3.776 | 0 | 0 | 0 | 0 | 0 | 0 |
| BRHIP2007741 | 1.447 | 0 | 0 | 0 | 0 | 0 | 0 |
| BRHIP2008269 | 23.203 | 0 | 0 | 0 | 0 | 0 | 0 |
| BRHIP2009685 | 0 | 0 | 0 | 73.716 | 0 | 3.990 | 0 |
| BRHIP2010571 | 12.840 | 0 | 0 | 0 | 0 | 0 | 0 |
| BRHIP2010593 | 0 | 12.963 | 0 | 0 | 0 | 0 | 0 |
| BRHIP2011199 | 44.974 | 0 | 0 | 0 | 0 | 0 | 0 |
| BRHIP2011508 | 54.020 | 0 | 0 | 0 | 0 | 0 | 0 |
| BRHIP2014373 | 28.142 | 0 | 0 | 0 | 0 | 0 | 0 |
| BRHIP2014386 | 44.974 | 0 | 0 | 0 | 0 | 0 | 0 |
| BRHIP2015153 | 0 | 0 | 0 | 0 | 0 | 11.223 | 0 |
| BRHIP2015245 | 9.898 | 0 | 0 | 0 | 0 | 5.318 | 0 |
| BRHIP2015679 | 2.092 | 0 | 0 | 0 | 0 | 0 | 0 |
| BRHIP2017010 | 0 | 0 | 0 | 0 | 0 | 76.989 | 0 |
| BRHIP2017945 | 2.688 | 0 | 0 | 0 | 0 | 0 | 0 |
| BRHIP2018712 | 1.689 | 7.496 | 0 | 0 | 0 | 4.537 | 0 |
| BRHIP2019819 | 0 | 32.096 | 0 | 0 | 0 | 0 | 0 |
| BRHIP2020509 | 15.614 | 0 | 0 | 0 | 0 | 0 | 0 |
| BRHIP2020573 | 0 | 0 | 0 | 0 | 0 | 10.137 | 0 |
| BRHIP2020799 | 4.220 | 0 | 0 | 0 | 0 | 0 | 0 |
| BRHIP2022228 | 7.619 | 0 | 0 | 0 | 0 | 30.706 | 0 |
| BRHIP2022326 | 1.419 | 3.149 | 0 | 0 | 0 | 1.906 | 0 |
| BRHIP2022986 | 2.373 | 0 | 0 | 0 | 0 | 0 | 0 |
| BRHIP2023272 | 0 | 0 | 0 | 0 | 0 | 0 | 72.484 |
| BRHIP2023309 | 0 | 43.183 | 0 | 0 | 0 | 13.067 | 0 |
| BRHIP2023369 | 0 | 64.693 | 0 | 0 | 0 | 0 | 0 |
| BRHIP2023438 | 0 | 0 | 0 | 0 | 0 | 28.574 | 0 |
| BRHIP2023888 | 3.375 | 1.362 | 0 | 0 | 0 | 1.649 | 0 |
| BRHIP2024046 | 0 | 0 | 0 | 0 | 0 | 68.709 | 0 |
| BRHIP2024398 | 14.041 | 0 | 0 | 0 | 0 | 9.431 | 0 |
| BRHIP2024742 | 0 | 0 | 0 | 0 | 0 | 5.588 | 0 |
| BRHIP2025245 | 0 | 0 | 0 | 0 | 0 | 52.334 | 0 |
| BRHIP2027017 | 0 | 0 | 0 | 0 | 0 | 12.895 | 0 |
| BRHIP2027077 | 1.684 | 0 | 0 | 0 | 0 | 4.525 | 0 |
| BRHIP3000017 | 0 | 0 | 0 | 0 | 0 | 9.878 | 0 |
| BRHIP3000111 | 0 | 3.790 | 0 | 0 | 0 | 1.147 | 0 |
| BRHIP3000131 | 9.186 | 0 | 0 | 0 | 0 | 4.113 | 0 |
| BRHIP3000475 | 0 | 0 | 0 | 0 | 0 | 24.219 | 0 |
| BRHIP3002114 | 32.523 | 0 | 0 | 0 | 0 | 0 | 0 |
| BRHIP3002141 | 26.166 | 0 | 0 | 0 | 0 | 0 | 0 |
| BRHIP3002691 | 0 | 30.700 | 0 | 0 | 0 | 0 | 0 |
| BRHIP3005673 | 21.480 | 0 | 0 | 0 | 0 | 0 | 0 |
| BRHIP3006294 | 33.251 | 0 | 0 | 0 | 0 | 0 | 0 |
| BRHIP3007223 | 0 | 0 | 0 | 0 | 0 | 6.775 | 0 |
| BRHIP3008405 | 14.670 | 0 | 0 | 0 | 0 | 19.707 | 0 |
| BRHIP3012997 | 0 | 3.431 | 0 | 0 | 0 | 0 | 0 |
| BRHIP3014675 | 15.812 | 0 | 0 | 0 | 0 | 0 | 0 |
| BRHIP3017855 | 6.386 | 0 | 0 | 0 | 0 | 4.289 | 0 |
| BRHIP3018278 | 32.523 | 0 | 0 | 0 | 0 | 0 | 0 |
| BRHIP3018784 | 5.779 | 0 | 0 | 0 | 0 | 0 | 0 |
| BRHIP3020046 | 0.845 | 3.749 | 0 | 0 | 0 | 1.135 | 0 |
| BRHIP3020733 | 44.974 | 0 | 0 | 0 | 0 | 0 | 0 |
| BRSSN2000097 | 0 | 25.630 | 0 | 0 | 0 | 0 | 0 |
| BRSSN2000197 | 18.303 | 0 | 0 | 0 | 0 | 0 | 0 |
| BRSSN2000244 | 0 | 49.863 | 0 | 0 | 0 | 0 | 0 |
| BRSSN2000295 | 0 | 25.637 | 0 | 0 | 0 | 0 | 0 |
| BRSSN2000498 | 2.737 | 0 | 0 | 0 | 0 | 2.451 | 23.500 |
| BRSSN2000684 | 0 | 17.348 | 0 | 0 | 0 | 0 | 0 |
| BRSSN2000832 | 10.039 | 0 | 0 | 0 | 0 | 0 | 0 |
| BRSSN2001579 | 0 | 49.863 | 0 | 0 | 0 | 0 | 0 |
| BRSSN2001869 | 2.517 | 0 | 0 | 0 | 0 | 6.762 | 0 |
| BRSSN2003012 | 0 | 0 | 0 | 0 | 0 | 4.846 | 0 |
| BRSSN2004719 | 0 | 19.395 | 0 | 0 | 0 | 0 | 0 |
| BRSSN2005847 | 18.303 | 0 | 0 | 0 | 0 | 0 | 0 |
| BRSSN2006892 | 12.911 | 0 | 0 | 0 | 0 | 0 | 0 |
| BRSSN2007338 | 0 | 27.359 | 0 | 0 | 0 | 0 | 0 |
| BRSSN2007464 | 0 | 15.148 | 0 | 0 | 0 | 0 | 0 |
| BRSSN2009518 | 1.436 | 0 | 0 | 0 | 0 | 3.859 | 0 |
| BRSSN2010019 | 0 | 42.961 | 0 | 0 | 0 | 0 | 0 |
| BRSSN2011262 | 7.050 | 0 | 0 | 0 | 0 | 0 | 0 |
| BRSSN2011738 | 8.770 | 0 | 0 | 0 | 0 | 0 | 0 |
| BRSSN2011843 | 9.980 | 0 | 0 | 0 | 0 | 0 | 0 |
| BRSSN2012439 | 18.303 | 0 | 0 | 0 | 0 | 0 | 0 |
| BRSSN2013702 | 0 | 0 | 0 | 0 | 0 | 10.626 | 0 |
| BRSSN2014299 | 3.081 | 0 | 0 | 0 | 0 | 0 | 0 |
| BRSSN2014610 | 2.977 | 0 | 0 | 0 | 0 | 7.998 | 0 |
| BRSSN2014685 | 0 | 0 | 0 | 0 | 0 | 15.906 | 0 |
| BRSSN2015238 | 2.855 | 0 | 0 | 0 | 0 | 3.835 | 0 |
| BRSSN2018447 | 0 | 24.191 | 0 | 0 | 0 | 7.320 | 0 |
| BRSSN2018581 | 0 | 49.863 | 0 | 0 | 0 | 0 | 0 |
| BRSTN1000083 | 1.065 | 0 | 0 | 0 | 0 | 0.715 | 0 |
| BRSTN2000058 | 0 | 8.929 | 0 | 0 | 0 | 0 | 0 |
| BRSTN2001067 | 5.162 | 0 | 0 | 0 | 0 | 6.935 | 0 |
| BRSTN2004869 | 0 | 0 | 0 | 0 | 0 | 23.702 | 0 |
| BRSTN2010089 | 5.501 | 0 | 0 | 0 | 0 | 0 | 0 |
| BRSTN2010107 | 0 | 24.765 | 0 | 0 | 0 | 0 | 0 |
| BRSTN2011961 | 0.241 | 0 | 0 | 0 | 0 | 0.324 | 0 |
| BRSTN2012069 | 0.631 | 0.140 | 0.390 | 0 | 6.386 | 0.095 | 4.062 |
| BRSTN2012089 | 0 | 21.471 | 0 | 0 | 0 | 0 | 0 |
| BRSTN2012174 | 0.506 | 1.497 | 0 | 0 | 0 | 0.680 | 0 |
| BRSTN2012451 | 0 | 0 | 0 | 0 | 17.990 | 0 | 0 |
| BRSTN2013502 | 2.027 | 0 | 0 | 0 | 0 | 0 | 0 |
| BRSTN2013931 | 0 | 0 | 0 | 0 | 0 | 1.082 | 0 |
| BRSTN2014490 | 0 | 34.105 | 0 | 0 | 0 | 0 | 0 |
| BRSTN2015889 | 9.690 | 0 | 0 | 0 | 0 | 0 | 0 |
| BRSTN2015978 | 0 | 10.107 | 0 | 0 | 0 | 0 | 0 |
| BRSTN2016470 | 0.365 | 0.810 | 0 | 0 | 0 | 0 | 0 |
| BRSTN2016992 | 0.410 | 1.822 | 0 | 0 | 0 | 0.551 | 0 |
| BRSTN2017104 | 4.877 | 0 | 0 | 0 | 0 | 0 | 0 |
| BRSTN2017761 | 0 | 0 | 0 | 0 | 0 | 23.702 | 0 |
| BRTHA1000311 | 2.460 | 10.923 | 0 | 0 | 0 | 3.305 | 0 |
| BRTHA2005013 | 9.305 | 0 | 0 | 0 | 0 | 0 | 0 |
| BRTHA2005864 | 9.471 | 0 | 0 | 0 | 0 | 1.817 | 34.846 |
| BRTHA2007998 | 0 | 0 | 0 | 0 | 0 | 0.966 | 0 |
| BRTHA2008335 | 0 | 0 | 0 | 0 | 0 | 9.119 | 0 |
| BRTHA2009886 | 0 | 76.948 | 0 | 0 | 0 | 0 | 0 |
| BRTHA2011691 | 42.920 | 0 | 0 | 0 | 0 | 0 | 0 |
| BRTHA2012714 | 0 | 4.640 | 0 | 0 | 0 | 0 | 0 |
| BRTHA2012980 | 0 | 0 | 86.110 | 0 | 0 | 0 | 0 |
| BRTHA2013054 | 0 | 0 | 0 | 0 | 0 | 13.457 | 0 |
| BRTHA2013275 | 0 | 0 | 0 | 0 | 0 | 50.251 | 0 |
| BRTHA2013540 | 28.262 | 0 | 0 | 0 | 0 | 0 | 0 |
| BRTHA2014663 | 14.773 | 65.580 | 0 | 0 | 0 | 0 | 0 |
| BRTHA2014997 | 42.920 | 0 | 0 | 0 | 0 | 0 | 0 |
| BRTHA2016577 | 1.967 | 0 | 0 | 0 | 0 | 0 | 0 |
| BRTHA2016918 | 0 | 0 | 67.390 | 0 | 0 | 0 | 0 |
| BRTHA2017985 | 0 | 0 | 0 | 0 | 0 | 50.251 | 0 |
| BRTHA2018504 | 28.335 | 0 | 0 | 0 | 0 | 0 | 0 |
| BRTHA2018707 | 42.920 | 0 | 0 | 0 | 0 | 0 | 0 |
| BRTHA2019073 | 42.920 | 0 | 0 | 0 | 0 | 0 | 0 |
| BRTHA2021440 | 42.920 | 0 | 0 | 0 | 0 | 0 | 0 |
| BRTHA2026071 | 30.706 | 0 | 0 | 0 | 0 | 0 | 0 |
| BRTHA2035743 | 11.317 | 0 | 0 | 0 | 0 | 7.601 | 0 |
| BRTHA3000273 | 42.920 | 0 | 0 | 0 | 0 | 0 | 0 |
| BRTHA3002427 | 0 | 10.582 | 19.655 | 0 | 0 | 0 | 0 |
| BRTHA3002864 | 42.920 | 0 | 0 | 0 | 0 | 0 | 0 |
| BRTHA3003417 | 0 | 0 | 0 | 0 | 0 | 34.473 | 0 |
| BRTHA3003490 | 6.768 | 0 | 0 | 0 | 0 | 0 | 34.864 |
| BRTHA3003736 | 5.381 | 0 | 0 | 0 | 0 | 0 | 0 |
| BRTHA3004307 | 5.024 | 0 | 0 | 0 | 0 | 6.749 | 0 |
| BRTHA3007472 | 3.254 | 0 | 0 | 0 | 0 | 0 | 0 |
| BRTHA3010530 | 23.316 | 0 | 0 | 0 | 0 | 0 | 0 |
| BRTHA3011194 | 0 | 0 | 0 | 0 | 0 | 3.959 | 0 |
| BRTHA3011265 | 0 | 0 | 0 | 0 | 0 | 9.557 | 0 |
| BRTHA3020314 | 0 | 0 | 0 | 0 | 0 | 22.812 | 0 |
| BRTHA3021971 | 3.237 | 0 | 0 | 0 | 0 | 0 | 0 |
| BRTHA3022641 | 14.751 | 0 | 0 | 0 | 0 | 0 | 0 |
| CD34C3000252 | 0 | 3.038 | 0 | 0 | 0 | 0.919 | 0 |
| CHONS2000797 | 3.627 | 0 | 0 | 0 | 0 | 0 | 0 |
| CHONS2002829 | 4.087 | 0 | 0 | 0 | 0 | 3.660 | 0 |
| COLON2002443 | 3.760 | 0 | 0 | 0 | 0 | 0 | 0 |
| CTONG1000009 | 8.100 | 0 | 0 | 0 | 0 | 10.881 | 0 |
| CTONG1000010 | 5.406 | 0 | 0 | 0 | 0 | 0 | 0 |
| CTONG1000052 | 8.565 | 0 | 0 | 0 | 0 | 4.061 | 0 |
| CTONG1000087 | 0 | 0 | 0 | 0 | 0 | 3.514 | 0 |
| CTONG1000094 | 0.649 | 1.442 | 0 | 0 | 0 | 0.436 | 0 |
| CTONG1000175 | 9.827 | 0 | 0 | 0 | 0 | 5.658 | 0 |
| CTONG1000218 | 0.803 | 0 | 0 | 0 | 0 | 2.157 | 0 |
| CTONG1000241 | 8.844 | 0 | 0 | 0 | 0 | 0 | 0 |
| CTONG1000288 | 3.805 | 0 | 0 | 0 | 0 | 0 | 0 |
| CTONG1000467 | 0 | 0 | 0 | 0 | 0 | 19.250 | 0 |
| CTONG2000063 | 0 | 7.924 | 0 | 0 | 0 | 4.796 | 0 |
| CTONG2000201 | 0 | 0 | 0 | 0 | 0 | 23.884 | 0 |
| CTONG2000411 | 0 | 0 | 0 | 0 | 0 | 6.446 | 0 |
| CTONG2000465 | 30.924 | 0 | 0 | 0 | 0 | 0 | 0 |
| CTONG2000469 | 0 | 0 | 0 | 0 | 0 | 6.542 | 0 |
| CTONG2000508 | 1.317 | 2.923 | 5.429 | 0 | 0 | 9.730 | 0 |
| CTONG2000561 | 0 | 0 | 0 | 0 | 0 | 8.129 | 0 |
| CTONG2001366 | 6.463 | 0 | 0 | 0 | 0 | 0 | 0 |
| CTONG2001428 | 0 | 0 | 0 | 0 | 0 | 37.554 | 0 |
| CTONG2001748 | 3.486 | 0 | 0 | 0 | 0 | 4.683 | 0 |
| CTONG2001858 | 0 | 0 | 0 | 0 | 0 | 8.267 | 0 |
| CTONG2002073 | 3.664 | 0 | 0 | 0 | 0 | 1.641 | 0 |
| CTONG2002270 | 5.750 | 0 | 0 | 0 | 0 | 6.536 | 0 |
| CTONG2002395 | 30.924 | 0 | 0 | 0 | 0 | 0 | 0 |
| CTONG2002721 | 30.924 | 0 | 0 | 0 | 0 | 0 | 0 |
| CTONG2002744 | 2.946 | 0 | 4.049 | 0 | 0 | 2.639 | 0 |
| CTONG2002803 | 1.228 | 0 | 0 | 0 | 0 | 0 | 0 |
| CTONG2002820 | 9.379 | 0 | 0 | 0 | 0 | 0 | 0 |
| CTONG2002903 | 0 | 0 | 0 | 0 | 0 | 3.450 | 0 |
| CTONG2003094 | 0 | 13.564 | 0 | 0 | 0 | 4.105 | 0 |
| CTONG2003100 | 0 | 0 | 0 | 0 | 0 | 0 | 92.019 |
| CTONG2003298 | 0 | 0 | 0 | 0 | 0 | 7.316 | 0 |
| CTONG2003524 | 1.344 | 5.965 | 0 | 0 | 0 | 7.220 | 0 |
| CTONG2004423 | 0 | 0 | 0 | 0 | 0 | 12.872 | 0 |
| CTONG2004487 | 0.789 | 0 | 6.503 | 0 | 0 | 7.417 | 0 |
| CTONG2005028 | 0 | 0 | 0 | 0 | 0 | 7.668 | 0 |
| CTONG2005278 | 7.209 | 0 | 0 | 0 | 0 | 0 | 0 |
| CTONG2005468 | 0 | 0 | 0 | 0 | 0 | 16.699 | 0 |
| CTONG2005553 | 1.791 | 0 | 0 | 0 | 0 | 1.604 | 0 |
| CTONG2005615 | 0 | 0 | 0 | 0 | 0 | 63.916 | 0 |
| CTONG2006004 | 0.880 | 0 | 0 | 0 | 22.990 | 2.365 | 0 |
| CTONG2006235 | 1.366 | 6.064 | 0 | 0 | 0 | 9.174 | 0 |
| CTONG2006377 | 29.368 | 0 | 0 | 0 | 0 | 0 | 0 |
| CTONG2006562 | 1.167 | 0 | 0 | 0 | 0 | 1.568 | 0 |
| CTONG2006666 | 21.848 | 0 | 0 | 0 | 0 | 29.349 | 0 |
| CTONG2006709 | 2.156 | 0 | 0 | 0 | 0 | 2.897 | 0 |
| CTONG2006798 | 8.046 | 0 | 0 | 0 | 0 | 10.808 | 0 |
| CTONG2006932 | 0 | 0 | 7.565 | 0 | 0 | 0 | 0 |
| CTONG2007007 | 0 | 3.446 | 0 | 0 | 0 | 3.128 | 0 |
| CTONG2007072 | 7.175 | 0 | 0 | 0 | 0 | 9.639 | 0 |
| CTONG2007078 | 3.197 | 0 | 0 | 0 | 0 | 4.294 | 0 |
| CTONG2007168 | 0 | 0 | 0 | 0 | 0 | 20.314 | 0 |
| CTONG2007400 | 0 | 0 | 0 | 0 | 0 | 12.889 | 0 |
| CTONG2007417 | 35.948 | 0 | 0 | 0 | 0 | 0 | 0 |
| CTONG2007441 | 0 | 0 | 0 | 0 | 0 | 23.118 | 0 |
| CTONG2007500 | 1.881 | 0 | 0 | 0 | 0 | 0.842 | 0 |
| CTONG2007586 | 0 | 0 | 0 | 0 | 0 | 6.732 | 0 |
| CTONG2007776 | 3.547 | 0 | 0 | 0 | 0 | 0 | 0 |
| CTONG2008398 | 0 | 0 | 0 | 0 | 0 | 1.713 | 16.419 |
| CTONG2008466 | 6.519 | 0 | 0 | 0 | 0 | 0 | 0 |
| CTONG2008506 | 4.708 | 0 | 0 | 0 | 0 | 4.216 | 0 |
| CTONG2008621 | 0 | 0 | 0 | 0 | 0 | 37.554 | 0 |
| CTONG2008720 | 22.512 | 0 | 0 | 0 | 0 | 0 | 0 |
| CTONG2008792 | 0 | 27.199 | 0 | 0 | 0 | 0 | 0 |
| CTONG2009534 | 0 | 0 | 0 | 0 | 0 | 8.614 | 0 |
| CTONG2009844 | 0 | 66.526 | 0 | 0 | 0 | 0 | 0 |
| CTONG2009938 | 16.683 | 0 | 0 | 0 | 0 | 0 | 0 |
| CTONG2009963 | 34.474 | 0 | 0 | 0 | 0 | 0 | 0 |
| CTONG2010348 | 4.430 | 0 | 0 | 0 | 0 | 1.983 | 0 |
| CTONG2010408 | 14.289 | 0 | 0 | 0 | 0 | 0 | 0 |
| CTONG2010649 | 0 | 5.788 | 0 | 0 | 0 | 1.752 | 0 |
| CTONG2010652 | 13.402 | 0 | 0 | 0 | 0 | 0 | 0 |
| CTONG2010821 | 8.938 | 0 | 0 | 0 | 0 | 0.800 | 0 |
| CTONG2011801 | 2.469 | 0 | 0 | 0 | 0 | 0 | 0 |
| CTONG2011825 | 0 | 0 | 0 | 0 | 0 | 19.233 | 0 |
| CTONG2012001 | 11.925 | 0 | 0 | 0 | 0 | 0 | 0 |
| CTONG2012050 | 0 | 0 | 0 | 0 | 0 | 23.871 | 0 |
| CTONG2012401 | 0 | 0 | 0 | 0 | 0 | 4.712 | 0 |
| CTONG2012422 | 30.924 | 0 | 0 | 0 | 0 | 0 | 0 |
| CTONG2012452 | 0.594 | 1.318 | 0 | 7.369 | 0 | 1.595 | 0 |
| CTONG2012453 | 18.839 | 0 | 0 | 0 | 0 | 0 | 0 |
| CTONG2012607 | 1.442 | 0 | 0 | 0 | 0 | 0 | 0 |
| CTONG2012661 | 34.944 | 0 | 0 | 0 | 0 | 0 | 0 |
| CTONG2012745 | 4.625 | 0 | 0 | 0 | 0 | 0 | 0 |
| CTONG2012847 | 0 | 0 | 0 | 0 | 0 | 3.858 | 0 |
| CTONG2012996 | 7.304 | 0 | 0 | 0 | 0 | 3.271 | 0 |
| CTONG2013348 | 4.587 | 0 | 0 | 0 | 0 | 0 | 0 |
| CTONG2013907 | 0 | 0 | 0 | 0 | 0 | 2.415 | 0 |
| CTONG2013934 | 11.568 | 0 | 0 | 0 | 0 | 7.770 | 0 |
| CTONG2014119 | 60.822 | 0 | 0 | 0 | 0 | 0 | 0 |
| CTONG2014165 | 4.015 | 0 | 0 | 0 | 0 | 0 | 0 |
| CTONG2014280 | 20.963 | 0 | 0 | 0 | 0 | 0 | 0 |
| CTONG2014389 | 0 | 0 | 0 | 0 | 0 | 2.959 | 56.729 |
| CTONG2014491 | 0 | 0 | 0 | 0 | 0 | 37.554 | 0 |
| CTONG2014898 | 4.810 | 0 | 0 | 0 | 0 | 0 | 0 |
| CTONG2014954 | 20.390 | 0 | 0 | 0 | 0 | 13.695 | 0 |
| CTONG2015091 | 0.677 | 6.012 | 0 | 0 | 0 | 0 | 0 |
| CTONG2015345 | 7.118 | 0 | 0 | 0 | 0 | 19.125 | 0 |
| CTONG2015678 | 0 | 0 | 0 | 0 | 0 | 37.554 | 0 |
| CTONG2016056 | 1.658 | 0 | 0 | 0 | 0 | 0 | 0 |
| CTONG2016185 | 0 | 0 | 0 | 0 | 0 | 26.208 | 0 |
| CTONG2016505 | 0 | 66.526 | 0 | 0 | 0 | 0 | 0 |
| CTONG2016575 | 0.829 | 0 | 0 | 0 | 0 | 5.012 | 0 |
| CTONG2016869 | 37.440 | 0 | 0 | 0 | 0 | 2.959 | 0 |
| CTONG2016931 | 2.545 | 0 | 0 | 0 | 0 | 0 | 0 |
| CTONG2017429 | 0 | 0 | 0 | 0 | 0 | 16.168 | 0 |
| CTONG2017500 | 2.209 | 0 | 0 | 0 | 0 | 5.935 | 0 |
| CTONG2017798 | 49.854 | 0 | 0 | 0 | 0 | 0 | 0 |
| CTONG2017998 | 8.355 | 0 | 0 | 0 | 0 | 8.418 | 0 |
| CTONG2018211 | 2.844 | 0 | 0 | 0 | 0 | 0 | 0 |
| CTONG2018334 | 0 | 0 | 0 | 0 | 0 | 8.193 | 0 |
| CTONG2018629 | 0 | 0 | 0 | 0 | 0 | 23.118 | 0 |
| CTONG2018637 | 0 | 0 | 0 | 0 | 0 | 7.349 | 0 |
| CTONG2018735 | 30.924 | 0 | 0 | 0 | 0 | 0 | 0 |
| CTONG2018739 | 22.620 | 0 | 0 | 0 | 0 | 0 | 0 |
| CTONG2018843 | 9.257 | 0 | 0 | 0 | 0 | 0 | 0 |
| CTONG2018898 | 0.402 | 3.565 | 0 | 0 | 0 | 1.618 | 0 |
| CTONG2018976 | 30.924 | 0 | 0 | 0 | 0 | 0 | 0 |
| CTONG2019029 | 0 | 0 | 0 | 0 | 0 | 9.064 | 0 |
| CTONG2019248 | 0 | 0 | 0 | 0 | 0 | 16.946 | 0 |
| CTONG2019590 | 8.973 | 0 | 0 | 0 | 0 | 0 | 0 |
| CTONG2019704 | 0 | 0 | 0 | 0 | 0 | 37.554 | 0 |
| CTONG2019788 | 0 | 0 | 0 | 0 | 0 | 3.425 | 0 |
| CTONG2019822 | 2.711 | 0 | 0 | 0 | 0 | 3.642 | 0 |
| CTONG2020108 | 30.924 | 0 | 0 | 0 | 0 | 0 | 0 |
| CTONG2020378 | 0 | 0 | 0 | 0 | 0 | 18.079 | 0 |
| CTONG2020484 | 0 | 2.176 | 0 | 0 | 0 | 0 | 0 |
| CTONG2020560 | 0 | 0 | 0 | 0 | 0 | 8.402 | 0 |
| CTONG2020582 | 1.600 | 0 | 0 | 0 | 0 | 6.448 | 0 |
| CTONG2020638 | 0 | 0 | 0 | 0 | 0 | 27.737 | 0 |
| CTONG2021024 | 0 | 0 | 0 | 0 | 0 | 37.554 | 0 |
| CTONG2021289 | 34.318 | 0 | 0 | 0 | 0 | 11.525 | 0 |
| CTONG2022835 | 0 | 0 | 0 | 0 | 0 | 1.922 | 0 |
| CTONG2024031 | 0 | 0 | 0 | 0 | 0 | 2.895 | 0 |
| CTONG2027327 | 47.240 | 0 | 0 | 0 | 0 | 0 | 0 |
| CTONG2028124 | 0.838 | 0 | 3.455 | 0 | 0 | 1.689 | 0 |
| CTONG2028309 | 24.482 | 0 | 0 | 0 | 0 | 0 | 0 |
| CTONG2028758 | 0 | 59.532 | 0 | 0 | 0 | 0 | 0 |
| CTONG3000657 | 0 | 14.362 | 0 | 0 | 0 | 0 | 0 |
| CTONG3001123 | 6.543 | 0 | 0 | 0 | 0 | 0 | 0 |
| CTONG3001746 | 0 | 0 | 0 | 0 | 0 | 16.024 | 0 |
| CTONG3002127 | 0 | 0 | 0 | 0 | 0 | 37.554 | 0 |
| CTONG3002518 | 7.036 | 0 | 0 | 0 | 0 | 9.452 | 0 |
| CTONG3002674 | 18.629 | 0 | 0 | 0 | 0 | 0 | 0 |
| CTONG3002835 | 19.238 | 0 | 0 | 0 | 0 | 3.692 | 0 |
| CTONG3003905 | 16.329 | 0 | 0 | 0 | 0 | 0 | 0 |
| CTONG3004072 | 0 | 0 | 0 | 0 | 0 | 43.729 | 0 |
| CTONG3004576 | 0 | 0 | 0 | 0 | 0 | 37.554 | 0 |
| CTONG3008894 | 2.425 | 0 | 0 | 0 | 0 | 0.814 | 0 |
| CTONG3009028 | 4.026 | 0 | 0 | 0 | 0 | 3.605 | 0 |
| D30ST2000734 | 6.666 | 0 | 0 | 0 | 0 | 0 | 0 |
| D30ST2001670 | 3.111 | 0 | 0 | 0 | 0 | 0 | 0 |
| D30ST2001788 | 2.432 | 0 | 0 | 0 | 63.521 | 0 | 0 |
| D30ST2002068 | 0 | 0 | 0 | 0 | 0 | 0 | 4.614 |
| D30ST3000258 | 5.183 | 0 | 0 | 0 | 0 | 0 | 0 |
| D30ST3000291 | 6.666 | 0 | 0 | 0 | 0 | 0 | 0 |
| D60ST2000358 | 0 | 0 | 0 | 0 | 0 | 1.592 | 0 |
| D60ST2000464 | 0.914 | 0 | 0 | 0 | 0 | 0 | 0 |
| D90ST2002608 | 5.573 | 8.247 | 0 | 0 | 0 | 7.487 | 0 |
| D90ST2003791 | 10.203 | 0 | 0 | 0 | 0 | 0 | 0 |
| DFNES1000083 | 0 | 0 | 0 | 0 | 0 | 2.520 | 0 |
| DFNES1000150 | 1.862 | 0 | 7.678 | 0 | 0 | 3.753 | 0 |
| DFNES2000011 | 1.824 | 6.073 | 0 | 0 | 0 | 4.900 | 0 |
| DFNES2000426 | 10.529 | 0 | 0 | 0 | 0 | 0 | 0 |
| DFNES2000443 | 0 | 2.513 | 0 | 0 | 0 | 0 | 0 |
| DFNES2000526 | 2.304 | 0 | 0 | 0 | 0 | 0 | 0 |
| DFNES2001404 | 0 | 38.579 | 0 | 0 | 0 | 0 | 0 |
| DFNES2003742 | 0 | 38.579 | 0 | 0 | 0 | 0 | 0 |
| DFNES2004346 | 0 | 0 | 0 | 0 | 68.168 | 0 | 0 |
| DFNES2004371 | 2.631 | 0 | 0 | 0 | 0 | 5.302 | 0 |
| DFNES2005540 | 2.521 | 0 | 0 | 0 | 0 | 0 | 0 |
| DFNES2005690 | 0 | 0 | 0 | 0 | 0 | 5.687 | 0 |
| DFNES2005766 | 0 | 0 | 0 | 0 | 0 | 2.054 | 0 |
| DFNES2006346 | 0 | 0 | 0 | 0 | 0 | 15.826 | 0 |
| DFNES2007113 | 0 | 0 | 0 | 0 | 0 | 7.485 | 0 |
| DFNES2007332 | 3.095 | 0 | 25.515 | 0 | 0 | 0 | 0 |
| DFNES2007634 | 2.965 | 3.290 | 0 | 0 | 0 | 0 | 0 |
| DFNES2008088 | 0 | 9.731 | 0 | 0 | 0 | 0 | 0 |
| DFNES2008160 | 3.736 | 0 | 0 | 0 | 0 | 0 | 0 |
| DFNES2009482 | 0 | 0 | 0 | 0 | 30.893 | 0 | 0 |
| DFNES2011221 | 0 | 0 | 0 | 0 | 0 | 15.971 | 0 |
| DFNES2011499 | 0 | 0 | 0 | 0 | 0 | 6.949 | 0 |
| FCBBF1000024 | 1.831 | 0 | 0 | 0 | 0 | 4.919 | 0 |
| FCBBF1000025 | 0 | 11.846 | 0 | 0 | 0 | 3.585 | 0 |
| FCBBF1000027 | 2.138 | 2.712 | 0 | 0 | 0 | 1.231 | 0 |
| FCBBF1000038 | 9.368 | 0 | 0 | 0 | 0 | 0 | 0 |
| FCBBF1000053 | 2.257 | 10.018 | 18.606 | 0 | 0 | 3.031 | 0 |
| FCBBF1000061 | 1.961 | 8.706 | 16.169 | 0 | 0 | 2.634 | 0 |
| FCBBF1000063 | 4.987 | 0 | 0 | 0 | 0 | 13.399 | 0 |
| FCBBF1000077 | 0 | 2.998 | 0 | 0 | 0 | 0 | 0 |
| FCBBF1000115 | 0 | 0 | 0 | 0 | 0 | 22.769 | 0 |
| FCBBF1000139 | 0 | 6.471 | 0 | 0 | 0 | 3.916 | 0 |
| FCBBF1000197 | 2.672 | 0 | 0 | 0 | 0 | 4.786 | 0 |
| FCBBF1000294 | 6.284 | 0 | 7.402 | 0 | 0 | 1.206 | 0 |
| FCBBF1000367 | 1.267 | 0 | 0 | 0 | 0 | 0 | 0 |
| FCBBF1000412 | 0 | 0 | 0 | 0 | 0 | 6.503 | 0 |
| FCBBF1000425 | 0 | 0 | 0 | 0 | 0 | 23.391 | 0 |
| FCBBF1000466 | 3.186 | 2.357 | 0 | 0 | 0 | 13.554 | 0 |
| FCBBF1000509 | 0 | 6.907 | 0 | 0 | 0 | 0 | 0 |
| FCBBF1000556 | 0 | 3.946 | 0 | 0 | 0 | 0 | 0 |
| FCBBF1000563 | 0 | 0 | 0 | 0 | 0 | 3.365 | 0 |
| FCBBF1000574 | 12.116 | 0 | 0 | 0 | 0 | 0 | 0 |
| FCBBF1000687 | 0 | 0 | 0 | 0 | 0 | 9.676 | 0 |
| FCBBF1000760 | 2.730 | 0 | 0 | 0 | 0 | 0.734 | 0 |
| FCBBF2000087 | 0 | 0 | 0 | 0 | 0 | 3.231 | 0 |
| FCBBF2000199 | 2.333 | 0 | 0 | 0 | 0 | 0 | 0 |
| FCBBF2000782 | 31.099 | 0 | 0 | 0 | 0 | 0 | 0 |
| FCBBF2000885 | 1.280 | 0 | 0 | 0 | 0 | 5.158 | 0 |
| FCBBF2001088 | 13.520 | 0 | 0 | 0 | 0 | 0 | 0 |
| FCBBF2001183 | 0 | 12.243 | 0 | 0 | 0 | 0 | 0 |
| FCBBF2001291 | 3.173 | 0 | 0 | 0 | 0 | 2.131 | 0 |
| FCBBF2001309 | 3.628 | 16.105 | 0 | 0 | 0 | 0 | 0 |
| FCBBF2001400 | 16.981 | 0 | 0 | 0 | 0 | 22.812 | 0 |
| FCBBF2001538 | 27.955 | 5.641 | 0 | 0 | 0 | 0 | 0 |
| FCBBF2002349 | 0 | 0 | 0 | 0 | 0 | 4.422 | 0 |
| FCBBF2002801 | 6.492 | 0 | 0 | 0 | 0 | 0 | 0 |
| FCBBF2002928 | 0 | 0 | 0 | 0 | 0 | 0 | 66.822 |
| FCBBF2005909 | 5.204 | 0 | 0 | 0 | 0 | 0 | 0 |
| FCBBF2007556 | 7.620 | 0 | 0 | 0 | 0 | 0 | 0 |
| FCBBF3000110 | 0 | 0 | 0 | 0 | 0 | 7.234 | 0 |
| FCBBF3000115 | 13.311 | 0 | 0 | 0 | 0 | 0 | 0 |
| FCBBF3000228 | 7.473 | 0 | 0 | 0 | 0 | 0 | 0 |
| FCBBF3000434 | 1.544 | 6.853 | 0 | 0 | 0 | 0 | 0 |
| FCBBF3000536 | 4.445 | 0 | 0 | 0 | 0 | 2.986 | 0 |
| FCBBF3001081 | 4.379 | 0 | 0 | 0 | 0 | 2.941 | 0 |
| FCBBF3001302 | 2.772 | 0 | 0 | 0 | 0 | 0 | 0 |
| FCBBF3001470 | 2.264 | 0 | 0 | 0 | 0 | 0 | 0 |
| FCBBF3001818 | 0 | 0 | 0 | 0 | 0 | 20.406 | 0 |
| FCBBF3001890 | 0 | 20.364 | 0 | 0 | 0 | 0 | 0 |
| FCBBF3003800 | 0 | 2.916 | 0 | 0 | 0 | 1.765 | 0 |
| FCBBF3004473 | 2.874 | 0 | 0 | 0 | 0 | 0 | 0 |
| FCBBF3004715 | 3.122 | 0 | 0 | 0 | 81.563 | 0 | 0 |
| FCBBF3005320 | 2.275 | 0 | 0 | 0 | 0 | 0 | 0 |
| FCBBF3005330 | 1.733 | 0 | 0 | 0 | 0 | 4.656 | 0 |
| FCBBF3005497 | 5.244 | 0 | 0 | 0 | 0 | 0.881 | 0 |
| FCBBF3006821 | 4.767 | 0 | 0 | 0 | 0 | 12.806 | 0 |
| FCBBF3007244 | 12.945 | 0 | 0 | 0 | 0 | 0 | 0 |
| FCBBF3008153 | 13.686 | 0 | 0 | 0 | 0 | 6.128 | 0 |
| FCBBF3008382 | 0 | 39.870 | 0 | 0 | 0 | 0 | 0 |
| FCBBF3009256 | 31.099 | 0 | 0 | 0 | 0 | 0 | 0 |
| FCBBF3009541 | 0 | 0 | 2.834 | 0 | 0 | 1.847 | 0 |
| FCBBF3010665 | 21.935 | 0 | 0 | 0 | 0 | 29.466 | 0 |
| FCBBF3010695 | 0 | 9.180 | 0 | 0 | 0 | 2.778 | 0 |
| FCBBF3011523 | 0 | 66.707 | 0 | 0 | 0 | 0 | 0 |
| FCBBF3012347 | 1.830 | 0 | 0 | 0 | 0 | 2.459 | 0 |
| FCBBF3013266 | 0 | 0 | 0 | 0 | 0 | 23.680 | 0 |
| FCBBF3016667 | 0 | 47.387 | 0 | 0 | 0 | 0 | 0 |
| FCBBF3016928 | 0 | 3.243 | 0 | 0 | 0 | 3.926 | 0 |
| FCBBF3017024 | 0 | 0 | 0 | 0 | 0 | 11.482 | 0 |
| FCBBF3017531 | 1.337 | 0 | 0 | 0 | 0 | 0 | 0 |
| FCBBF3017918 | 5.460 | 0 | 0 | 0 | 0 | 7.334 | 0 |
| FCBBF3018796 | 2.589 | 11.492 | 0 | 0 | 0 | 0 | 0 |
| FCBBF3019320 | 49.450 | 0 | 0 | 0 | 0 | 0 | 0 |
| FCBBF3019839 | 6.245 | 13.863 | 0 | 0 | 0 | 0 | 0 |
| FCBBF3020140 | 47.443 | 0 | 0 | 0 | 0 | 0 | 0 |
| FCBBF3022504 | 12.995 | 0 | 0 | 0 | 0 | 0 | 0 |
| FCBBF3022593 | 0 | 25.647 | 0 | 0 | 0 | 0 | 0 |
| FCBBF3022767 | 16.981 | 0 | 0 | 0 | 0 | 22.812 | 0 |
| FCBBF3023368 | 0 | 0 | 0 | 0 | 0 | 2.748 | 0 |
| FCBBF3023667 | 0.332 | 0.492 | 0.913 | 0 | 2.893 | 0.149 | 0 |
| FCBBF3024002 | 4.732 | 0 | 0 | 0 | 0 | 6.357 | 0 |
| FCBBF3024793 | 0 | 59.143 | 0 | 0 | 0 | 0 | 0 |
| FCBBF3025737 | 0 | 0 | 0 | 0 | 0 | 3.640 | 0 |
| FCBBF3025905 | 40.015 | 0 | 0 | 0 | 0 | 0 | 0 |
| FCBBF3026308 | 10.657 | 0 | 0 | 0 | 0 | 0 | 0 |
| FCBBF3026651 | 0 | 0 | 0 | 0 | 0 | 8.174 | 0 |
| FCBBF3028202 | 0 | 42.529 | 0 | 0 | 0 | 0 | 0 |
| FCBBF3028244 | 2.323 | 3.437 | 0 | 0 | 0 | 3.120 | 0 |
| FCBBF3028593 | 6.733 | 0 | 0 | 0 | 0 | 0 | 0 |
| FCBBF4000017 | 7.406 | 0 | 0 | 0 | 0 | 0 | 0 |
| FCBBF4000446 | 0 | 2.780 | 0 | 0 | 0 | 1.683 | 0 |
| FCBBF4000548 | 0 | 0 | 0 | 0 | 0 | 16.372 | 0 |
| FCBBF5000261 | 4.329 | 0 | 35.696 | 0 | 0 | 0 | 0 |
| FEBRA1000057 | 0 | 6.426 | 0 | 0 | 0 | 0 | 0 |
| FEBRA2000105 | 1.196 | 0 | 19.724 | 0 | 0 | 2.410 | 0 |
| FEBRA2000129 | 4.335 | 0 | 0 | 0 | 0 | 2.912 | 0 |
| FEBRA2000210 | 1.768 | 0 | 0 | 0 | 0 | 0.792 | 0 |
| FEBRA2000228 | 3.294 | 0 | 0 | 0 | 0 | 4.425 | 0 |
| FEBRA2000253 | 12.206 | 0 | 0 | 0 | 0 | 0 | 0 |
| FEBRA2000377 | 8.342 | 0 | 0 | 0 | 0 | 0 | 0 |
| FEBRA2000402 | 4.622 | 0 | 0 | 0 | 0 | 0 | 0 |
| FEBRA2000438 | 14.734 | 0 | 0 | 0 | 0 | 0 | 0 |
| FEBRA2000452 | 0 | 3.713 | 0 | 0 | 21.846 | 4.494 | 0 |
| FEBRA2000454 | 25.065 | 0 | 0 | 0 | 0 | 0 | 0 |
| FEBRA2000510 | 3.077 | 0 | 0 | 0 | 0 | 4.133 | 0 |
| FEBRA2000532 | 0 | 0 | 0 | 66.176 | 0 | 0 | 0 |
| FEBRA2000536 | 0 | 11.660 | 0 | 0 | 0 | 3.528 | 0 |
| FEBRA2000733 | 0.141 | 0 | 0 | 0 | 0 | 0 | 0 |
| FEBRA2000740 | 3.772 | 0 | 0 | 0 | 0 | 5.067 | 0 |
| FEBRA2000757 | 2.553 | 0 | 0 | 0 | 0 | 0 | 0 |
| FEBRA2000787 | 4.941 | 0 | 0 | 0 | 0 | 0 | 0 |
| FEBRA2000815 | 0 | 13.382 | 0 | 0 | 0 | 0 | 0 |
| FEBRA2000881 | 0 | 0 | 0 | 0 | 0 | 3.546 | 0 |
| FEBRA2001146 | 18.649 | 0 | 0 | 0 | 0 | 12.526 | 0 |
| FEBRA2001227 | 0 | 13.686 | 0 | 0 | 0 | 0 | 0 |
| FEBRA2001294 | 2.015 | 0 | 0 | 0 | 0 | 2.707 | 0 |
| FEBRA2001492 | 2.699 | 0 | 0 | 0 | 0 | 0 | 0 |
| FEBRA2001523 | 0 | 0 | 58.455 | 0 | 0 | 0 | 0 |
| FEBRA2001571 | 1.749 | 15.525 | 0 | 0 | 0 | 2.349 | 0 |
| FEBRA2001581 | 3.419 | 0 | 0 | 0 | 0 | 0 | 0 |
| FEBRA2001669 | 0.893 | 0 | 0 | 0 | 0 | 0 | 0 |
| FEBRA2001698 | 0 | 45.061 | 0 | 0 | 0 | 0 | 0 |
| FEBRA2001715 | 0 | 3.691 | 0 | 0 | 0 | 0 | 0 |
| FEBRA2001745 | 0 | 0 | 0 | 0 | 0 | 1.404 | 0 |
| FEBRA2001814 | 0 | 9.795 | 0 | 0 | 0 | 1.482 | 0 |
| FEBRA2001828 | 4.537 | 0 | 0 | 0 | 0 | 0 | 0 |
| FEBRA2001867 | 6.181 | 13.719 | 0 | 0 | 0 | 0 | 0 |
| FEBRA2001974 | 19.431 | 0 | 0 | 0 | 0 | 13.051 | 0 |
| FEBRA2001989 | 25.065 | 0 | 0 | 0 | 0 | 0 | 0 |
| FEBRA2001990 | 0 | 13.433 | 0 | 0 | 0 | 0 | 0 |
| FEBRA2002352 | 0 | 12.850 | 0 | 0 | 0 | 0 | 0 |
| FEBRA2002429 | 3.265 | 0 | 0 | 0 | 0 | 0 | 0 |
| FEBRA2002456 | 0 | 4.037 | 0 | 0 | 0 | 0 | 0 |
| FEBRA2002574 | 25.065 | 0 | 0 | 0 | 0 | 0 | 0 |
| FEBRA2002601 | 0 | 0 | 73.389 | 0 | 0 | 0 | 0 |
| FEBRA2002707 | 0 | 0 | 0 | 0 | 0 | 4.036 | 0 |
| FEBRA2002792 | 0 | 0 | 9.202 | 0 | 0 | 0 | 0 |
| FEBRA2002933 | 0.309 | 0 | 0 | 0 | 0 | 0.415 | 0 |
| FEBRA2003115 | 0 | 0 | 0 | 0 | 0 | 24.282 | 0 |
| FEBRA2003181 | 25.065 | 0 | 0 | 0 | 0 | 0 | 0 |
| FEBRA2003258 | 0 | 0 | 0 | 0 | 0 | 3.169 | 0 |
| FEBRA2003436 | 4.635 | 0 | 0 | 0 | 0 | 0 | 0 |
| FEBRA2003726 | 0.830 | 0 | 0 | 0 | 0 | 0 | 0 |
| FEBRA2003750 | 0 | 6.758 | 0 | 0 | 0 | 8.179 | 0 |
| FEBRA2003833 | 1.442 | 0 | 0 | 0 | 0 | 3.874 | 0 |
| FEBRA2003926 | 1.164 | 0 | 0 | 0 | 0 | 0.782 | 0 |
| FEBRA2003930 | 1.499 | 0 | 0 | 0 | 0 | 0 | 0 |
| FEBRA2004042 | 1.769 | 0 | 0 | 0 | 0 | 0 | 0 |
| FEBRA2004293 | 6.650 | 0 | 0 | 0 | 0 | 5.955 | 0 |
| FEBRA2004331 | 25.065 | 0 | 0 | 0 | 0 | 0 | 0 |
| FEBRA2004485 | 0 | 25.690 | 0 | 0 | 0 | 0 | 0 |
| FEBRA2004628 | 0 | 10.559 | 0 | 0 | 0 | 0 | 0 |
| FEBRA2004818 | 0 | 0 | 0 | 0 | 0 | 4.576 | 0 |
| FEBRA2005114 | 0 | 24.839 | 0 | 0 | 0 | 0 | 0 |
| FEBRA2005291 | 0 | 0 | 0 | 0 | 0 | 0 | 77.883 |
| FEBRA2005361 | 0 | 5.185 | 0 | 0 | 0 | 0 | 0 |
| FEBRA2005416 | 1.791 | 0 | 0 | 0 | 0 | 0 | 0 |
| FEBRA2005476 | 0 | 0 | 0 | 0 | 0 | 0 | 55.937 |
| FEBRA2005572 | 11.886 | 0 | 0 | 0 | 0 | 1.996 | 0 |
| FEBRA2005734 | 0.704 | 0 | 0 | 0 | 0 | 0 | 0 |
| FEBRA2005778 | 0 | 0 | 0 | 0 | 0 | 3.360 | 0 |
| FEBRA2005787 | 25.065 | 0 | 0 | 0 | 0 | 0 | 0 |
| FEBRA2005824 | 0 | 5.523 | 0 | 0 | 0 | 0 | 0 |
| FEBRA2005995 | 0 | 15.568 | 0 | 0 | 0 | 0 | 0 |
| FEBRA2006092 | 0 | 0 | 0 | 0 | 0 | 1.393 | 0 |
| FEBRA2006255 | 0 | 4.823 | 0 | 0 | 0 | 0 | 0 |
| FEBRA2006270 | 5.849 | 0 | 0 | 0 | 0 | 0 | 0 |
| FEBRA2006357 | 0 | 2.452 | 0 | 0 | 0 | 0 | 0 |
| FEBRA2006368 | 0 | 11.502 | 0 | 0 | 0 | 0 | 0 |
| FEBRA2006372 | 2.240 | 0 | 9.233 | 0 | 0 | 1.504 | 0 |
| FEBRA2006401 | 0 | 0 | 0 | 0 | 0 | 1.125 | 0 |
| FEBRA2006485 | 2.537 | 0 | 0 | 0 | 0 | 3.407 | 0 |
| FEBRA2006491 | 37.148 | 0 | 0 | 0 | 0 | 0 | 0 |
| FEBRA2006553 | 0 | 14.645 | 0 | 0 | 0 | 0 | 0 |
| FEBRA2006808 | 0 | 15.195 | 0 | 0 | 0 | 0 | 0 |
| FEBRA2006873 | 3.744 | 0 | 0 | 0 | 0 | 0 | 0 |
| FEBRA2007212 | 0 | 14.531 | 0 | 0 | 0 | 4.397 | 0 |
| FEBRA2007544 | 0 | 0 | 0 | 0 | 0 | 5.485 | 0 |
| FEBRA2007613 | 5.534 | 12.283 | 0 | 0 | 0 | 7.434 | 0 |
| FEBRA2007622 | 1.721 | 0 | 7.093 | 0 | 0 | 2.311 | 0 |
| FEBRA2007801 | 0 | 0 | 0 | 0 | 0 | 2.765 | 0 |
| FEBRA2007880 | 18.830 | 0 | 0 | 0 | 0 | 0 | 0 |
| FEBRA2007900 | 6.463 | 0 | 0 | 0 | 0 | 8.682 | 0 |
| FEBRA2007901 | 3.090 | 0 | 0 | 0 | 0 | 0 | 0 |
| FEBRA2008036 | 0 | 5.287 | 0 | 0 | 0 | 0 | 0 |
| FEBRA2008081 | 2.362 | 2.621 | 0 | 14.654 | 0 | 3.173 | 0 |
| FEBRA2008087 | 0.299 | 0 | 2.465 | 3.709 | 11.713 | 0 | 0 |
| FEBRA2008201 | 3.999 | 0 | 0 | 0 | 0 | 0 | 0 |
| FEBRA2008266 | 0 | 67.271 | 0 | 0 | 0 | 0 | 0 |
| FEBRA2008287 | 0 | 10.911 | 0 | 0 | 0 | 0 | 0 |
| FEBRA2008302 | 0 | 7.879 | 0 | 0 | 0 | 0 | 0 |
| FEBRA2008341 | 10.955 | 0 | 0 | 0 | 0 | 0 | 0 |
| FEBRA2008583 | 0 | 3.425 | 0 | 0 | 0 | 0.592 | 0 |
| FEBRA2008662 | 0 | 17.626 | 0 | 0 | 0 | 0 | 0 |
| FEBRA2008692 | 0 | 0 | 0 | 0 | 0 | 27.483 | 52.692 |
| FEBRA2008755 | 2.739 | 0 | 0 | 0 | 0 | 0 | 70.553 |
| FEBRA2009185 | 0 | 13.453 | 0 | 0 | 0 | 0 | 0 |
| FEBRA2009419 | 2.204 | 4.891 | 0 | 0 | 0 | 2.960 | 0 |
| FEBRA2009514 | 2.762 | 0 | 0 | 0 | 0 | 0 | 0 |
| FEBRA2009780 | 0 | 0 | 0 | 0 | 0 | 31.003 | 0 |
| FEBRA2009986 | 10.719 | 0 | 0 | 0 | 0 | 0 | 0 |
| FEBRA2011090 | 14.472 | 0 | 0 | 0 | 0 | 0 | 0 |
| FEBRA2012120 | 0 | 59.757 | 0 | 0 | 0 | 0 | 0 |
| FEBRA2012181 | 13.767 | 0 | 0 | 0 | 0 | 0 | 0 |
| FEBRA2013699 | 11.935 | 0 | 0 | 0 | 0 | 0 | 0 |
| FEBRA2014010 | 0 | 59.757 | 0 | 0 | 0 | 0 | 0 |
| FEBRA2014213 | 0 | 0 | 0 | 0 | 85.773 | 4.411 | 0 |
| FEBRA2014297 | 1.782 | 0 | 0 | 0 | 0 | 0 | 45.899 |
| FEBRA2014417 | 0.900 | 1.999 | 3.712 | 0 | 0 | 1.210 | 0 |
| FEBRA2014578 | 18.250 | 0 | 0 | 0 | 0 | 0 | 0 |
| FEBRA2015085 | 1.271 | 0 | 0 | 0 | 0 | 0 | 0 |
| FEBRA2016572 | 16.384 | 0 | 0 | 0 | 0 | 0 | 0 |
| FEBRA2017024 | 0 | 35.715 | 0 | 0 | 0 | 0 | 0 |
| FEBRA2017680 | 0 | 35.198 | 0 | 0 | 0 | 0 | 0 |
| FEBRA2018476 | 1.822 | 0 | 0 | 0 | 0 | 1.224 | 0 |
| FEBRA2018746 | 6.236 | 0 | 0 | 0 | 0 | 0 | 0 |
| FEBRA2019242 | 6.765 | 0 | 0 | 0 | 0 | 0 | 0 |
| FEBRA2019663 | 7.263 | 0 | 0 | 0 | 0 | 9.756 | 0 |
| FEBRA2019690 | 0 | 59.757 | 0 | 0 | 0 | 0 | 0 |
| FEBRA2021032 | 8.941 | 0 | 0 | 0 | 0 | 0 | 0 |
| FEBRA2022504 | 1.369 | 0 | 0 | 0 | 0 | 0 | 0 |
| FEBRA2023227 | 4.271 | 0 | 0 | 0 | 0 | 0 | 0 |
| FEBRA2023927 | 6.792 | 0 | 0 | 0 | 0 | 13.687 | 0 |
| FEBRA2024019 | 0 | 9.928 | 0 | 0 | 0 | 0 | 0 |
| FEBRA2024136 | 28.699 | 0 | 0 | 0 | 0 | 0 | 0 |
| FEBRA2025427 | 0 | 0 | 0 | 0 | 0 | 2.777 | 0 |
| FEBRA2025838 | 0.685 | 0 | 5.648 | 0 | 0 | 0 | 0 |
| FEBRA2028158 | 0 | 0 | 0 | 0 | 0 | 31.003 | 0 |
| FEBRA2028516 | 0 | 4.234 | 0 | 0 | 0 | 2.562 | 0 |
| FEKID1000018 | 0 | 14.612 | 0 | 0 | 0 | 0 | 0 |
| FELIV1000101 | 0.259 | 0 | 0 | 0 | 0 | 0 | 0 |
| FELIV1000137 | 0 | 0 | 0 | 0 | 0 | 0.347 | 0 |
| FELIV1000153 | 0 | 0 | 2.268 | 3.413 | 3.592 | 0 | 0 |
| FELIV1000164 | 0 | 0 | 0 | 0 | 0 | 0.651 | 0 |
| HCASM1000115 | 0.864 | 0 | 0 | 0 | 0 | 0 | 0 |
| HCASM2000016 | 2.489 | 2.763 | 0 | 0 | 0 | 3.344 | 0 |
| HCASM2000138 | 3.522 | 0 | 0 | 0 | 0 | 4.731 | 0 |
| HCASM2000363 | 0.567 | 0 | 0 | 0 | 0 | 0 | 0 |
| HCASM2003018 | 7.359 | 0 | 0 | 0 | 0 | 0 | 0 |
| HCASM2005388 | 2.949 | 0 | 0 | 0 | 0 | 3.962 | 0 |
| HCHON1000030 | 1.210 | 2.686 | 1.247 | 0 | 0 | 0.203 | 3.895 |
| HCHON1000166 | 2.508 | 0 | 0 | 0 | 0 | 0 | 0 |
| HCHON2000028 | 1.323 | 5.873 | 0 | 0 | 0 | 0 | 0 |
| HCHON2000056 | 0.709 | 0 | 1.950 | 0 | 0 | 2.224 | 0 |
| HCHON2000062 | 0 | 0 | 0 | 0 | 0 | 4.695 | 0 |
| HCHON2000087 | 0.516 | 0 | 0 | 0 | 0 | 2.772 | 0 |
| HCHON2000160 | 0.839 | 0 | 0 | 0 | 0 | 1.128 | 0 |
| HCHON2000226 | 0 | 0 | 0 | 0 | 0 | 0.903 | 8.660 |
| HCHON2000265 | 1.687 | 0 | 0 | 0 | 0 | 0 | 0 |
| HCHON2000295 | 0 | 3.243 | 0 | 0 | 0 | 0 | 0 |
| HCHON2000323 | 1.545 | 0 | 0 | 0 | 40.348 | 0 | 0 |
| HCHON2000364 | 0 | 0 | 0 | 0 | 0 | 0.498 | 0 |
| HCHON2000473 | 0 | 0 | 0 | 0 | 0 | 13.113 | 0 |
| HCHON2000508 | 2.180 | 1.382 | 0 | 0 | 0 | 0.837 | 0 |
| HCHON2000626 | 0.980 | 0 | 0 | 0 | 0 | 0 | 0 |
| HCHON2000698 | 2.390 | 0 | 0 | 0 | 0 | 3.211 | 0 |
| HCHON2000699 | 11.644 | 0 | 0 | 0 | 0 | 0 | 0 |
| HCHON2000738 | 0 | 0 | 0 | 0 | 0 | 3.335 | 0 |
| HCHON2000743 | 0 | 0 | 0 | 0 | 0 | 13.678 | 0 |
| HCHON2000751 | 8.931 | 0 | 0 | 0 | 0 | 0 | 0 |
| HCHON2000826 | 1.194 | 0 | 0 | 0 | 0 | 2.405 | 0 |
| HCHON2000832 | 0 | 13.096 | 0 | 0 | 0 | 0 | 0 |
| HCHON2001039 | 22.330 | 0 | 0 | 0 | 0 | 6.249 | 0 |
| HCHON2001084 | 1.915 | 0 | 0 | 0 | 0 | 2.572 | 0 |
| HCHON2001200 | 1.443 | 1.601 | 0 | 0 | 0 | 0.484 | 0 |
| HCHON2001217 | 2.604 | 0 | 0 | 0 | 0 | 3.498 | 0 |
| HCHON2001359 | 1.420 | 0 | 0 | 0 | 0 | 0.954 | 0 |
| HCHON2001434 | 3.821 | 0 | 0 | 0 | 0 | 1.711 | 0 |
| HCHON2001665 | 0 | 0 | 0 | 0 | 0 | 5.186 | 0 |
| HCHON2001712 | 11.644 | 0 | 0 | 0 | 0 | 0 | 0 |
| HCHON2001853 | 0 | 0 | 38.504 | 0 | 0 | 0 | 0 |
| HCHON2002247 | 2.341 | 0 | 0 | 0 | 0 | 0 | 0 |
| HCHON2002354 | 10.594 | 0 | 0 | 0 | 0 | 0 | 0 |
| HCHON2002971 | 11.644 | 0 | 0 | 0 | 0 | 0 | 0 |
| HCHON2004002 | 3.273 | 0 | 0 | 0 | 0 | 3.518 | 0 |
| HCHON2005987 | 0 | 36.909 | 0 | 0 | 0 | 0 | 0 |
| HCHON2006250 | 0 | 0 | 0 | 32.381 | 0 | 0 | 0 |
| HCHON2006459 | 0 | 0 | 0 | 0 | 0 | 12.090 | 0 |
| HCHON2007650 | 5.078 | 0 | 0 | 0 | 0 | 0 | 0 |
| HCHON2008856 | 0 | 36.909 | 0 | 0 | 0 | 0 | 0 |
| HEART1000074 | 3.165 | 0 | 0 | 0 | 0 | 0.850 | 0 |
| HEART1000149 | 0 | 3.476 | 0 | 0 | 0 | 1.052 | 0 |
| HEART2000506 | 0 | 0 | 0 | 0 | 0 | 1.964 | 0 |
| HEART2006195 | 0 | 0 | 0 | 0 | 0 | 10.616 | 0 |
| HEART2006521 | 0 | 0 | 0 | 0 | 0 | 2.679 | 0 |
| HEART2007231 | 8.078 | 0 | 0 | 0 | 0 | 0 | 0 |
| HHDPC1000065 | 7.625 | 0 | 0 | 0 | 0 | 0 | 0 |
| HHDPC1000083 | 0.816 | 0 | 0 | 0 | 0 | 0 | 0 |
| HHDPC1000163 | 0 | 0 | 8.646 | 0 | 0 | 0 | 0 |
| HHDPC2000055 | 0 | 0 | 0 | 0 | 39.829 | 2.048 | 0 |
| HHDPC2000070 | 8.024 | 0 | 0 | 0 | 0 | 0 | 0 |
| HHDPC2000104 | 0 | 0 | 0 | 0 | 0 | 2.072 | 0 |
| HHDPC2000149 | 3.726 | 0 | 0 | 0 | 0 | 0 | 0 |
| HHDPC2000455 | 0.458 | 0 | 0 | 0 | 0 | 1.231 | 0 |
| HHDPC2000456 | 1.295 | 0 | 0 | 0 | 0 | 3.478 | 0 |
| HHDPC2000572 | 0 | 0 | 0 | 0 | 0 | 22.001 | 0 |
| HHDPC2003439 | 0.424 | 0 | 0 | 0 | 0 | 0.285 | 0 |
| HHDPC2003983 | 0.929 | 1.031 | 3.829 | 0 | 0 | 0.936 | 0 |
| HHDPC2005742 | 10.588 | 0 | 0 | 0 | 0 | 0 | 0 |
| HHDPC2007267 | 0 | 0 | 0 | 0 | 10.634 | 3.828 | 0 |
| HHDPC2008123 | 10.588 | 0 | 0 | 0 | 0 | 0 | 0 |
| HHDPC2009272 | 0 | 0 | 0 | 0 | 0 | 13.725 | 0 |
| HLUNG1000037 | 0 | 0 | 0 | 0 | 0 | 9.843 | 0 |
| HLUNG1000055 | 4.062 | 2.004 | 3.721 | 0 | 0 | 0.606 | 11.625 |
| HLUNG1000076 | 0.701 | 0 | 0 | 0 | 0 | 0 | 0 |
| HLUNG1000099 | 0 | 0 | 0 | 0 | 0 | 12.090 | 0 |
| HLUNG2000004 | 0 | 0 | 0 | 0 | 0 | 5.667 | 0 |
| HLUNG2000063 | 4.113 | 0 | 0 | 0 | 0 | 0 | 0 |
| HLUNG2000125 | 0 | 0 | 0 | 73.829 | 0 | 0 | 0 |
| HLUNG2000176 | 18.523 | 0 | 0 | 0 | 0 | 0 | 0 |
| HLUNG2000412 | 8.931 | 0 | 0 | 0 | 0 | 2.999 | 0 |
| HLUNG2000751 | 0 | 0 | 0 | 0 | 0 | 19.353 | 0 |
| HLUNG2000846 | 0 | 0 | 0 | 0 | 0 | 5.061 | 0 |
| HLUNG2000884 | 0 | 0 | 0 | 0 | 0 | 2.398 | 0 |
| HLUNG2000950 | 10.309 | 0 | 0 | 0 | 0 | 0 | 0 |
| HLUNG2001146 | 0 | 0 | 0 | 0 | 0 | 11.743 | 0 |
| HLUNG2001439 | 2.206 | 0 | 0 | 0 | 0 | 0 | 0 |
| HLUNG2001518 | 6.383 | 0 | 0 | 0 | 0 | 4.899 | 0 |
| HLUNG2001633 | 0 | 0 | 0 | 0 | 0 | 17.937 | 0 |
| HLUNG2001677 | 0 | 0 | 0 | 0 | 76.536 | 0 | 0 |
| HLUNG2001712 | 2.749 | 0 | 0 | 0 | 0 | 0 | 0 |
| HLUNG2002059 | 0 | 0 | 0 | 0 | 0 | 12.273 | 0 |
| HLUNG2002465 | 0 | 0 | 0 | 0 | 0 | 1.355 | 0 |
| HLUNG2002648 | 6.028 | 0 | 0 | 0 | 0 | 0 | 0 |
| HLUNG2003003 | 6.664 | 0 | 0 | 0 | 0 | 0 | 0 |
| HLUNG2003042 | 0 | 0 | 0 | 0 | 0 | 2.666 | 0 |
| HLUNG2003306 | 0 | 0 | 0 | 0 | 0 | 14.343 | 0 |
| HLUNG2003378 | 0 | 0 | 0 | 0 | 0 | 10.370 | 0 |
| HLUNG2003497 | 18.523 | 0 | 0 | 0 | 0 | 0 | 0 |
| HLUNG2004154 | 0 | 0 | 0 | 0 | 0 | 23.297 | 0 |
| HLUNG2004159 | 3.024 | 0 | 0 | 0 | 0 | 6.092 | 0 |
| HLUNG2004534 | 0 | 0 | 0 | 0 | 0 | 3.249 | 0 |
| HLUNG2005203 | 0 | 0 | 0 | 0 | 0 | 23.395 | 0 |
| HLUNG2006370 | 10.207 | 0 | 0 | 0 | 0 | 0 | 0 |
| HLUNG2006570 | 0 | 0 | 0 | 0 | 0 | 21.718 | 0 |
| HLUNG2006812 | 2.466 | 0 | 0 | 0 | 0 | 6.624 | 0 |
| HLUNG2007219 | 18.523 | 0 | 0 | 0 | 0 | 0 | 0 |
| HLUNG2008153 | 5.676 | 0 | 0 | 0 | 0 | 0 | 0 |
| HLUNG2008637 | 1.409 | 0 | 11.613 | 0 | 0 | 0 | 0 |
| HLUNG2009225 | 5.030 | 0 | 10.368 | 0 | 0 | 1.689 | 0 |
| HLUNG2011833 | 0 | 0 | 0 | 84.945 | 0 | 0 | 0 |
| HLUNG2014821 | 0 | 0 | 0 | 0 | 0 | 37.919 | 0 |
| HLUNG2015184 | 21.837 | 0 | 0 | 0 | 0 | 0 | 0 |
| HLUNG2015418 | 0 | 0 | 0 | 0 | 0 | 23.395 | 0 |
| HLUNG2017262 | 1.322 | 0 | 0 | 0 | 0 | 7.105 | 0 |
| HLUNG2017546 | 2.943 | 0 | 0 | 0 | 0 | 0 | 0 |
| HSYRA1000062 | 0.714 | 0 | 0 | 0 | 9.330 | 0.960 | 0 |
| HSYRA1000119 | 1.150 | 0 | 0 | 0 | 10.015 | 0.515 | 0 |
| HSYRA1000152 | 0 | 0 | 0 | 0 | 0 | 1.336 | 25.615 |
| HSYRA1000178 | 0 | 0 | 0 | 0 | 0 | 16.852 | 0 |
| HSYRA2000044 | 10.037 | 0 | 0 | 0 | 0 | 0 | 0 |
| HSYRA2000159 | 0.402 | 3.124 | 0.829 | 0 | 0 | 1.350 | 2.589 |
| HSYRA2000224 | 1.499 | 0 | 0 | 0 | 0 | 2.014 | 0 |
| HSYRA2000232 | 1.233 | 0 | 0 | 0 | 0 | 0 | 0 |
| HSYRA2000248 | 0.567 | 2.516 | 0 | 0 | 0 | 0.761 | 0 |
| HSYRA2000253 | 7.045 | 0 | 0 | 0 | 0 | 0 | 0 |
| HSYRA2000255 | 0 | 0 | 0 | 0 | 0 | 1.005 | 0 |
| HSYRA2000424 | 0 | 0 | 0 | 0 | 0 | 13.033 | 0 |
| HSYRA2000629 | 2.783 | 12.354 | 0 | 0 | 0 | 0 | 0 |
| HSYRA2000640 | 2.841 | 6.307 | 0 | 0 | 0 | 4.771 | 0 |
| HSYRA2000743 | 10.325 | 2.865 | 0 | 0 | 0 | 1.734 | 0 |
| HSYRA2000787 | 2.594 | 0 | 0 | 0 | 0 | 3.484 | 0 |
| HSYRA2000828 | 1.188 | 5.274 | 0 | 0 | 0 | 1.596 | 0 |
| HSYRA2000925 | 0.924 | 0 | 0 | 0 | 0 | 0 | 0 |
| HSYRA2001103 | 0 | 0 | 5.831 | 0 | 0 | 1.900 | 36.429 |
| HSYRA2001105 | 2.297 | 0 | 0 | 0 | 0 | 3.086 | 0 |
| HSYRA2001138 | 0.213 | 3.775 | 0 | 0 | 0 | 1.142 | 0 |
| HSYRA2001225 | 0.616 | 0 | 0 | 0 | 0 | 0 | 0 |
| HSYRA2001353 | 0 | 37.564 | 0 | 0 | 0 | 0 | 0 |
| HSYRA2001396 | 2.534 | 0 | 0 | 0 | 33.091 | 0 | 0 |
| HSYRA2001420 | 0 | 0 | 0 | 0 | 0 | 6.241 | 0 |
| HSYRA2001476 | 1.208 | 0 | 3.319 | 0 | 0 | 2.704 | 0 |
| HSYRA2001567 | 3.737 | 0 | 0 | 0 | 0 | 2.510 | 0 |
| HSYRA2001595 | 2.375 | 2.109 | 0 | 0 | 12.408 | 0.638 | 0 |
| HSYRA2001621 | 0 | 2.038 | 0 | 0 | 0 | 0.411 | 3.941 |
| HSYRA2001638 | 5.658 | 0 | 0 | 0 | 0 | 0 | 0 |
| HSYRA2001983 | 0 | 17.866 | 0 | 0 | 0 | 0 | 0 |
| IMR321000161 | 19.114 | 0 | 0 | 0 | 0 | 0 | 0 |
| IMR321000210 | 0 | 3.752 | 0 | 0 | 0 | 0 | 0 |
| IMR321000219 | 1.170 | 1.039 | 0 | 0 | 0 | 1.886 | 0 |
| IMR321000253 | 0 | 0 | 0 | 0 | 0 | 1.141 | 0 |
| IMR321000266 | 0 | 0 | 0 | 0 | 0 | 6.340 | 0 |
| IMR322000121 | 0 | 4.126 | 0 | 0 | 0 | 0 | 0 |
| IMR322000127 | 3.875 | 0 | 0 | 0 | 0 | 0 | 0 |
| IMR322000178 | 1.551 | 0 | 0 | 0 | 0 | 0 | 0 |
| IMR322000223 | 0.568 | 0 | 0 | 0 | 0 | 0.763 | 0 |
| IMR322000243 | 1.431 | 0 | 0 | 0 | 0 | 2.883 | 0 |
| IMR322000316 | 0 | 1.536 | 0 | 0 | 0 | 0.465 | 0 |
| IMR322000374 | 0 | 0 | 0 | 0 | 0 | 6.170 | 0 |
| IMR322000730 | 0.229 | 0 | 1.885 | 2.836 | 2.985 | 0.154 | 5.887 |
| IMR322000918 | 2.444 | 0 | 0 | 0 | 0 | 0 | 0 |
| IMR322000935 | 0 | 5.542 | 0 | 0 | 0 | 1.677 | 0 |
| IMR322000973 | 0.672 | 0 | 0 | 0 | 0 | 0 | 0 |
| IMR322001435 | 2.350 | 0 | 0 | 0 | 0 | 1.052 | 0 |
| IMR322001541 | 2.821 | 0 | 0 | 0 | 0 | 0 | 0 |
| IMR322001584 | 3.123 | 0 | 0 | 0 | 0 | 0 | 0 |
| IMR322001600 | 0.134 | 0 | 0 | 0 | 0 | 0.180 | 6.916 |
| IMR322001710 | 1.617 | 0 | 0 | 0 | 0 | 4.345 | 0 |
| IMR322013805 | 11.264 | 0 | 0 | 0 | 0 | 0 | 0 |
| IMR322019070 | 4.462 | 0 | 0 | 0 | 0 | 5.994 | 0 |
| JCMLC2000273 | 2.577 | 0 | 0 | 0 | 0 | 0 | 0 |
| KIDNE1000104 | 13.829 | 0 | 0 | 0 | 0 | 0 | 0 |
| KIDNE1000145 | 9.224 | 0 | 0 | 0 | 0 | 0 | 0 |
| KIDNE2000085 | 4.287 | 0 | 7.070 | 0 | 0 | 1.152 | 0 |
| KIDNE2000252 | 0.713 | 0 | 0 | 0 | 0 | 0.957 | 0 |
| KIDNE2000266 | 10.533 | 0 | 0 | 0 | 0 | 0 | 0 |
| KIDNE2000349 | 0 | 2.341 | 0 | 0 | 0 | 0 | 0 |
| KIDNE2000383 | 4.107 | 0 | 0 | 0 | 0 | 11.033 | 0 |
| KIDNE2000403 | 2.755 | 0 | 0 | 0 | 0 | 2.776 | 0 |
| KIDNE2000517 | 0 | 5.797 | 0 | 0 | 0 | 0 | 0 |
| KIDNE2000574 | 0 | 2.771 | 0 | 0 | 0 | 0 | 0 |
| KIDNE2000947 | 2.107 | 0 | 0 | 0 | 0 | 0 | 0 |
| KIDNE2001162 | 0.778 | 0 | 3.209 | 0 | 0 | 0.523 | 10.024 |
| KIDNE2001361 | 19.301 | 0 | 0 | 0 | 0 | 0 | 0 |
| KIDNE2001467 | 0.869 | 0 | 0 | 0 | 0 | 2.920 | 0 |
| KIDNE2001636 | 11.821 | 0 | 0 | 0 | 0 | 0 | 0 |
| KIDNE2001713 | 0 | 0 | 0 | 0 | 0 | 17.356 | 0 |
| KIDNE2001847 | 0 | 0 | 0 | 0 | 0 | 0 | 78.156 |
| KIDNE2002252 | 1.818 | 0 | 0 | 0 | 0 | 7.325 | 0 |
| KIDNE2002725 | 0 | 0 | 0 | 0 | 0 | 11.525 | 0 |
| KIDNE2002883 | 1.891 | 0 | 0 | 0 | 0 | 0 | 0 |
| KIDNE2003305 | 2.807 | 0 | 11.573 | 0 | 0 | 0 | 0 |
| KIDNE2003316 | 6.309 | 0 | 0 | 0 | 0 | 0 | 0 |
| KIDNE2003357 | 0 | 0 | 0 | 0 | 0 | 16.562 | 0 |
| KIDNE2003373 | 0 | 0 | 0 | 0 | 0 | 17.356 | 0 |
| KIDNE2003941 | 0 | 3.734 | 0 | 0 | 0 | 1.130 | 0 |
| KIDNE2003994 | 0 | 0 | 0 | 0 | 0 | 9.032 | 0 |
| KIDNE2004295 | 10.647 | 0 | 0 | 0 | 0 | 0 | 0 |
| KIDNE2004344 | 19.301 | 0 | 0 | 0 | 0 | 0 | 0 |
| KIDNE2004411 | 0 | 0 | 0 | 0 | 0 | 9.545 | 0 |
| KIDNE2004475 | 2.146 | 9.528 | 0 | 0 | 0 | 0 | 0 |
| KIDNE2004531 | 19.301 | 0 | 0 | 0 | 0 | 0 | 0 |
| KIDNE2004534 | 3.112 | 0 | 25.661 | 0 | 0 | 0 | 0 |
| KIDNE2004864 | 8.141 | 0 | 0 | 0 | 0 | 0 | 0 |
| KIDNE2005676 | 1.173 | 0 | 0 | 0 | 0 | 4.728 | 0 |
| KIDNE2006009 | 0 | 0 | 0 | 0 | 0 | 4.130 | 0 |
| KIDNE2006014 | 0 | 0 | 0 | 0 | 0 | 3.326 | 0 |
| KIDNE2006248 | 0 | 1.151 | 2.138 | 0 | 6.772 | 0 | 0 |
| KIDNE2006353 | 65.219 | 0 | 0 | 0 | 0 | 0 | 0 |
| KIDNE2006465 | 0 | 11.454 | 0 | 0 | 0 | 0 | 0 |
| KIDNE2006652 | 0 | 0 | 0 | 0 | 0 | 9.674 | 0 |
| KIDNE2006775 | 3.055 | 0 | 0 | 0 | 0 | 0 | 0 |
| KIDNE2006811 | 0 | 0 | 0 | 0 | 0 | 3.645 | 0 |
| KIDNE2006880 | 2.677 | 0 | 0 | 0 | 0 | 0 | 0 |
| KIDNE2007328 | 7.377 | 0 | 0 | 0 | 0 | 7.928 | 0 |
| KIDNE2007352 | 0.693 | 1.539 | 0 | 0 | 0 | 0 | 0 |
| KIDNE2007954 | 5.273 | 0 | 0 | 0 | 0 | 2.361 | 0 |
| KIDNE2008048 | 3.131 | 0 | 0 | 0 | 0 | 3.605 | 0 |
| KIDNE2008403 | 0 | 0 | 0 | 0 | 0 | 10.663 | 0 |
| KIDNE2008987 | 0 | 0 | 0 | 0 | 62.744 | 0 | 0 |
| KIDNE2009367 | 0.628 | 2.790 | 0 | 0 | 0 | 2.533 | 0 |
| KIDNE2009605 | 12.593 | 0 | 0 | 0 | 0 | 0 | 0 |
| KIDNE2010049 | 0 | 0 | 0 | 0 | 0 | 3.851 | 0 |
| KIDNE2011635 | 0 | 51.498 | 0 | 0 | 0 | 0 | 0 |
| KIDNE2012440 | 1.476 | 0 | 0 | 0 | 0 | 0 | 0 |
| KIDNE2012710 | 0 | 0 | 0 | 0 | 0 | 21.213 | 0 |
| KIDNE2013158 | 15.126 | 0 | 0 | 0 | 0 | 0 | 0 |
| KIDNE2013845 | 6.524 | 0 | 0 | 0 | 0 | 5.008 | 0 |
| KIDNE2014290 | 2.923 | 0 | 0 | 0 | 0 | 2.356 | 0 |
| KIDNE2014325 | 6.309 | 0 | 0 | 0 | 0 | 0 | 0 |
| KIDNE2014808 | 0 | 0 | 0 | 0 | 0 | 24.317 | 0 |
| KIDNE2015445 | 0 | 0 | 23.619 | 0 | 0 | 0 | 0 |
| KIDNE2015546 | 0 | 51.498 | 0 | 0 | 0 | 0 | 0 |
| KIDNE2015598 | 19.301 | 0 | 0 | 0 | 0 | 0 | 0 |
| KIDNE2016327 | 0 | 0 | 0 | 0 | 0 | 19.599 | 0 |
| KIDNE2016371 | 7.513 | 0 | 0 | 0 | 0 | 0 | 0 |
| KIDNE2017040 | 2.804 | 0 | 0 | 0 | 0 | 0 | 0 |
| KIDNE2017052 | 19.301 | 0 | 0 | 0 | 0 | 0 | 0 |
| KIDNE2018352 | 0 | 0 | 24.516 | 0 | 0 | 0 | 0 |
| KIDNE2018493 | 8.914 | 0 | 0 | 0 | 0 | 0 | 0 |
| KIDNE2018891 | 10.170 | 0 | 0 | 0 | 0 | 6.831 | 0 |
| LIVER1000017 | 0 | 0 | 19.417 | 0 | 0 | 0 | 0 |
| LIVER1000067 | 0 | 0 | 9.545 | 0 | 0 | 0 | 0 |
| LIVER1000079 | 0 | 0 | 12.046 | 0 | 0 | 1.963 | 0 |
| LIVER1000104 | 0 | 0 | 7.084 | 0 | 0 | 0 | 0 |
| LIVER1000111 | 0 | 23.377 | 0 | 0 | 0 | 0 | 0 |
| LIVER1000132 | 0.835 | 0 | 0 | 0 | 0 | 0 | 0 |
| LIVER1000230 | 0 | 0 | 0 | 0 | 0 | 6.496 | 0 |
| LIVER1000303 | 22.395 | 0 | 0 | 0 | 0 | 0 | 0 |
| LIVER2000037 | 0 | 0 | 7.394 | 0 | 0 | 0 | 0 |
| LIVER2001539 | 4.152 | 0 | 0 | 0 | 0 | 0 | 0 |
| LIVER2001608 | 1.095 | 0 | 9.029 | 0 | 0 | 1.471 | 0 |
| LIVER2002842 | 3.184 | 0 | 0 | 0 | 0 | 2.138 | 0 |
| LIVER2003065 | 0 | 0 | 44.231 | 0 | 0 | 0 | 0 |
| LIVER2004514 | 0 | 0 | 0 | 0 | 0 | 11.443 | 0 |
| LIVER2005789 | 0 | 0 | 6.189 | 0 | 0 | 0 | 0 |
| LIVER2006006 | 0 | 0 | 4.889 | 0 | 0 | 0 | 0 |
| LIVER2007783 | 0 | 7.595 | 0 | 0 | 0 | 0 | 0 |
| LIVER2008706 | 0 | 0 | 61.334 | 0 | 0 | 0 | 0 |
| MAMGL1000017 | 0.254 | 0 | 0 | 0 | 0 | 0 | 0 |
| MAMGL1000032 | 0 | 0 | 0 | 0 | 7.019 | 0 | 0 |
| MAMGL1000035 | 0.504 | 0 | 1.384 | 0 | 0 | 0 | 0 |
| MAMGL1000083 | 0 | 0 | 0 | 0 | 0 | 4.867 | 0 |
| MAMGL1000173 | 0.221 | 0 | 0 | 0 | 0 | 0.297 | 0 |
| MAMGL1000178 | 0.255 | 0 | 0 | 0 | 0 | 0 | 0 |
| MESAN1000050 | 0 | 5.942 | 0 | 0 | 0 | 5.395 | 0 |
| MESAN1000079 | 0 | 0 | 0 | 0 | 0 | 16.100 | 0 |
| MESAN1000101 | 4.813 | 0 | 0 | 0 | 0 | 6.465 | 0 |
| MESAN1000126 | 1.489 | 0 | 0 | 0 | 0 | 2.000 | 0 |
| MESAN2000092 | 0 | 0 | 0 | 0 | 0 | 9.912 | 0 |
| MESAN2000149 | 2.048 | 0 | 0 | 0 | 0 | 2.752 | 0 |
| MESAN2000267 | 1.366 | 0 | 0 | 0 | 0 | 0.459 | 0 |
| MESAN2000572 | 12.788 | 0 | 7.029 | 0 | 0 | 2.290 | 0 |
| MESAN2000608 | 0.656 | 2.911 | 0 | 0 | 0 | 0 | 0 |
| MESAN2000815 | 13.020 | 0 | 0 | 0 | 0 | 0 | 0 |
| MESAN2001450 | 0 | 0 | 0 | 59.680 | 0 | 0 | 0 |
| MESAN2001627 | 0 | 0 | 0 | 0 | 0 | 14.651 | 0 |
| MESAN2002086 | 0.694 | 1.541 | 0 | 0 | 0 | 1.865 | 0 |
| MESAN2002724 | 0 | 0 | 0 | 0 | 0 | 1.033 | 0 |
| MESAN2002790 | 4.079 | 0 | 0 | 0 | 0 | 0 | 0 |
| MESAN2002940 | 0 | 0 | 0 | 0 | 0 | 3.562 | 0 |
| MESAN2003035 | 11.952 | 0 | 0 | 0 | 0 | 0 | 0 |
| MESAN2003058 | 14.982 | 0 | 0 | 0 | 0 | 0 | 0 |
| MESAN2003190 | 14.756 | 0 | 0 | 0 | 0 | 0 | 0 |
| MESAN2003444 | 6.212 | 0 | 0 | 0 | 0 | 0 | 0 |
| MESAN2003622 | 0.501 | 0 | 4.132 | 0 | 0 | 0 | 0 |
| MESAN2005284 | 4.043 | 0 | 0 | 0 | 0 | 5.432 | 0 |
| MESAN2005303 | 0 | 29.326 | 0 | 0 | 0 | 17.748 | 0 |
| MESAN2005633 | 14.314 | 0 | 0 | 0 | 0 | 0 | 0 |
| MESAN2005689 | 2.587 | 0 | 0 | 0 | 0 | 0 | 0 |
| MESAN2005766 | 3.289 | 0 | 0 | 0 | 0 | 2.209 | 0 |
| MESAN2005957 | 0 | 0 | 0 | 0 | 0 | 11.061 | 0 |
| MESAN2005958 | 6.737 | 0 | 0 | 0 | 0 | 0 | 0 |
| MESAN2006401 | 1.508 | 0 | 0 | 0 | 0 | 2.026 | 0 |
| MESAN2006563 | 1.727 | 0 | 0 | 0 | 0 | 0 | 0 |
| MESAN2007755 | 0 | 33.863 | 0 | 0 | 0 | 0 | 0 |
| MESAN2008926 | 2.662 | 0 | 0 | 0 | 0 | 0 | 0 |
| MESAN2008936 | 3.317 | 0 | 0 | 0 | 0 | 4.456 | 0 |
| MESAN2009580 | 0.926 | 0 | 0 | 0 | 0 | 0 | 23.850 |
| MESAN2010664 | 0 | 32.125 | 0 | 0 | 0 | 0 | 0 |
| MESAN2011984 | 7.113 | 0 | 0 | 0 | 0 | 0 | 0 |
| MESAN2012054 | 2.127 | 0 | 0 | 0 | 0 | 0 | 0 |
| MESAN2013284 | 6.145 | 0 | 0 | 0 | 0 | 0 | 0 |
| MESAN2013908 | 5.753 | 0 | 0 | 0 | 0 | 0 | 0 |
| MESAN2014192 | 18.359 | 0 | 0 | 0 | 0 | 0 | 0 |
| MESAN2014412 | 16.049 | 0 | 0 | 0 | 0 | 0 | 0 |
| MESAN2016552 | 10.145 | 0 | 0 | 0 | 0 | 0 | 0 |
| MESAN2017133 | 7.316 | 0 | 0 | 0 | 0 | 0 | 0 |
| N1ESE2000698 | 3.542 | 0 | 0 | 0 | 0 | 0 | 0 |
| NB9N41000121 | 0.626 | 0 | 0 | 0 | 0 | 0 | 0 |
| NB9N41000340 | 0 | 0 | 0 | 0 | 0 | 1.625 | 0 |
| NB9N42000104 | 1.382 | 0 | 0 | 0 | 0 | 0.929 | 0 |
| NESOP2000504 | 3.771 | 0 | 0 | 0 | 0 | 0 | 0 |
| NESOP2001656 | 0 | 14.819 | 0 | 0 | 0 | 0 | 0 |
| NHNPC2000062 | 1.747 | 0 | 0 | 0 | 0 | 0 | 0 |
| NHNPC2000206 | 4.581 | 0 | 0 | 0 | 0 | 0 | 0 |
| NOVAR1000015 | 2.532 | 0 | 0 | 0 | 0 | 0 | 0 |
| NOVAR1000091 | 1.615 | 0 | 0 | 0 | 0 | 2.169 | 0 |
| NOVAR2000038 | 0.518 | 2.299 | 0 | 0 | 0 | 0.696 | 0 |
| NOVAR2000710 | 0 | 0 | 0 | 0 | 0 | 11.838 | 0 |
| NOVAR2002039 | 3.380 | 0 | 0 | 0 | 0 | 0 | 0 |
| NT2NE1000014 | 7.248 | 0 | 0 | 0 | 0 | 9.737 | 0 |
| NT2NE1000122 | 18.705 | 0 | 0 | 0 | 0 | 0 | 0 |
| NT2NE1000163 | 0.338 | 0 | 0 | 0 | 0 | 2.269 | 0 |
| NT2NE1000181 | 15.221 | 0 | 0 | 0 | 0 | 0 | 0 |
| NT2NE2000056 | 0.983 | 2.908 | 0 | 0 | 0 | 4.840 | 0 |
| NT2NE2000174 | 0 | 0 | 0 | 0 | 0 | 0 | 8.195 |
| NT2NE2000195 | 0 | 5.690 | 10.568 | 0 | 0 | 0 | 0 |
| NT2NE2000260 | 0 | 0.447 | 2.491 | 0 | 0 | 0 | 5.189 |
| NT2NE2000356 | 18.705 | 0 | 0 | 0 | 0 | 0 | 0 |
| NT2NE2000383 | 3.801 | 0 | 0 | 0 | 0 | 5.106 | 0 |
| NT2NE2000384 | 1.919 | 0 | 0 | 0 | 0 | 5.156 | 0 |
| NT2NE2000392 | 0 | 3.305 | 0 | 0 | 0 | 0 | 0 |
| NT2NE2000470 | 4.550 | 0 | 0 | 0 | 0 | 0 | 0 |
| NT2NE2000536 | 0 | 0 | 59.169 | 0 | 0 | 9.640 | 0 |
| NT2NE2000575 | 2.205 | 3.559 | 1.653 | 0 | 0 | 0.808 | 0 |
| NT2NE2000707 | 0.837 | 6.194 | 0 | 0 | 0 | 3.748 | 0 |
| NT2NE2000763 | 7.005 | 8.885 | 0 | 0 | 0 | 0 | 0 |
| NT2NE2000809 | 8.479 | 0 | 0 | 0 | 0 | 0 | 0 |
| NT2NE2000909 | 0 | 12.117 | 0 | 0 | 0 | 0 | 0 |
| NT2NE2000950 | 4.370 | 19.401 | 0 | 0 | 0 | 0 | 0 |
| NT2NE2000963 | 0.191 | 0 | 0 | 0 | 4.997 | 0 | 0 |
| NT2NE2001000 | 0.718 | 0 | 0 | 0 | 0 | 0 | 0 |
| NT2NE2001048 | 1.248 | 0 | 0 | 0 | 0 | 1.677 | 0 |
| NT2NE2001326 | 0 | 0 | 0 | 0 | 0 | 23.610 | 0 |
| NT2NE2001403 | 4.326 | 0 | 0 | 0 | 0 | 0 | 0 |
| NT2NE2001435 | 7.978 | 0 | 0 | 0 | 0 | 0 | 0 |
| NT2NE2001545 | 5.976 | 0 | 0 | 0 | 0 | 2.007 | 0 |
| NT2NE2001617 | 0 | 0 | 0 | 0 | 0 | 5.082 | 0 |
| NT2NE2001648 | 0 | 32.127 | 0 | 0 | 0 | 0 | 0 |
| NT2NE2001655 | 0 | 5.182 | 0 | 0 | 0 | 1.568 | 0 |
| NT2NE2001660 | 0 | 28.992 | 0 | 0 | 0 | 8.773 | 0 |
| NT2NE2001793 | 0.865 | 0 | 0 | 0 | 0 | 4.647 | 0 |
| NT2NE2002100 | 18.705 | 0 | 0 | 0 | 0 | 0 | 0 |
| NT2NE2002162 | 10.194 | 0 | 0 | 0 | 0 | 0 | 0 |
| NT2NE2003485 | 1.941 | 0 | 0 | 0 | 0 | 5.215 | 0 |
| NT2NE2003569 | 0 | 0 | 0 | 0 | 0 | 1.944 | 0 |
| NT2NE2003887 | 5.204 | 0 | 0 | 0 | 0 | 0 | 0 |
| NT2NE2004255 | 0 | 0 | 0 | 0 | 0 | 6.672 | 0 |
| NT2NE2004740 | 0 | 0 | 0 | 0 | 0 | 23.610 | 0 |
| NT2NE2005317 | 0 | 0 | 0 | 0 | 0 | 14.244 | 0 |
| NT2NE2005323 | 18.705 | 0 | 0 | 0 | 0 | 0 | 0 |
| NT2NE2005371 | 9.195 | 0 | 0 | 0 | 0 | 0 | 0 |
| NT2NE2005688 | 0 | 0 | 0 | 0 | 0 | 0.836 | 0 |
| NT2NE2005876 | 0 | 5.460 | 0 | 0 | 0 | 0 | 0 |
| NT2NE2006458 | 0 | 0 | 0 | 0 | 30.788 | 0 | 0 |
| NT2NE2006478 | 0 | 0 | 0 | 0 | 0 | 12.036 | 0 |
| NT2NE2006909 | 0.238 | 0 | 0 | 0 | 0 | 0.320 | 0 |
| NT2NE2007786 | 0 | 0 | 0 | 0 | 0 | 0 | 85.561 |
| NT2NE2010842 | 3.145 | 0 | 0 | 0 | 0 | 3.168 | 0 |
| NT2NE2011107 | 10.028 | 0 | 0 | 0 | 0 | 0 | 0 |
| NT2NE2011119 | 0 | 0 | 0 | 0 | 0 | 5.187 | 0 |
| NT2NE2012505 | 0 | 0 | 0 | 0 | 0 | 20.538 | 0 |
| NT2NE2014113 | 0 | 33.588 | 0 | 0 | 0 | 0 | 0 |
| NT2NE2015262 | 18.705 | 0 | 0 | 0 | 0 | 0 | 0 |
| NT2NE2015362 | 0 | 0 | 0 | 0 | 0 | 20.538 | 0 |
| NT2NE2015974 | 6.370 | 0 | 0 | 0 | 0 | 0 | 0 |
| NT2NE2018180 | 3.135 | 0 | 0 | 0 | 0 | 0 | 0 |
| NT2NE2019091 | 0 | 21.023 | 0 | 0 | 0 | 0 | 0 |
| NT2RI1000016 | 0 | 0 | 0 | 0 | 0 | 28.303 | 0 |
| NT2RI1000027 | 1.686 | 0 | 0 | 0 | 0 | 0 | 0 |
| NT2RI1000048 | 2.124 | 0 | 0 | 0 | 0 | 0 | 0 |
| NT2RI1000127 | 0 | 0 | 0 | 0 | 0 | 2.033 | 0 |
| NT2RI2000064 | 0.449 | 0 | 0 | 0 | 0 | 0.603 | 0 |
| NT2RI2000107 | 3.863 | 0 | 0 | 0 | 0 | 5.189 | 0 |
| NT2RI2000116 | 0 | 2.153 | 0 | 0 | 0 | 0 | 0 |
| NT2RI2000167 | 0 | 42.766 | 0 | 0 | 0 | 0 | 0 |
| NT2RI2000255 | 0 | 4.431 | 0 | 0 | 0 | 0 | 0 |
| NT2RI2000263 | 0 | 0 | 0 | 0 | 37.259 | 1.916 | 0 |
| NT2RI2000282 | 0 | 0 | 0 | 0 | 0 | 0.355 | 0 |
| NT2RI2000284 | 0 | 3.375 | 0 | 0 | 0 | 0 | 9.790 |
| NT2RI2000294 | 0 | 0 | 0 | 0 | 41.225 | 0 | 0 |
| NT2RI2000348 | 7.389 | 0 | 0 | 0 | 0 | 0 | 0 |
| NT2RI2000412 | 6.427 | 0 | 0 | 0 | 0 | 0 | 0 |
| NT2RI2000578 | 0 | 1.238 | 0 | 0 | 0 | 0 | 0 |
| NT2RI2000671 | 0.877 | 0 | 0 | 0 | 0 | 1.178 | 0 |
| NT2RI2000685 | 5.783 | 0 | 0 | 0 | 0 | 0 | 0 |
| NT2RI2000727 | 0 | 2.873 | 0 | 0 | 0 | 0.290 | 5.556 |
| NT2RI2000775 | 0 | 0 | 0 | 0 | 0 | 19.103 | 0 |
| NT2RI2000822 | 8.883 | 0 | 0 | 0 | 0 | 0 | 0 |
| NT2RI2000829 | 0 | 28.277 | 0 | 0 | 0 | 0 | 0 |
| NT2RI2000974 | 0 | 0 | 0 | 0 | 0 | 2.416 | 0 |
| NT2RI2001010 | 31.453 | 0 | 0 | 0 | 0 | 0 | 0 |
| NT2RI2001342 | 1.316 | 0 | 0 | 0 | 0 | 0 | 0 |
| NT2RI2001422 | 0 | 13.761 | 0 | 0 | 0 | 0 | 0 |
| NT2RI2001540 | 0.997 | 0 | 0.783 | 0 | 6.200 | 0.255 | 1.223 |
| NT2RI2001657 | 4.015 | 0 | 0 | 0 | 0 | 0 | 0 |
| NT2RI2001726 | 8.805 | 0 | 0 | 0 | 0 | 0 | 0 |
| NT2RI2001731 | 3.158 | 0 | 0 | 0 | 0 | 0 | 0 |
| NT2RI2001846 | 0 | 0 | 0 | 0 | 0 | 11.040 | 0 |
| NT2RI2001859 | 4.855 | 0 | 0 | 0 | 0 | 0 | 0 |
| NT2RI2001866 | 12.100 | 0 | 0 | 0 | 0 | 16.255 | 0 |
| NT2RI2001910 | 6.228 | 0 | 0 | 0 | 0 | 0 | 0 |
| NT2RI2002091 | 0.157 | 0.099 | 0.553 | 1.110 | 0.876 | 0.346 | 0.576 |
| NT2RI2002152 | 0 | 4.655 | 0 | 0 | 0 | 0 | 0 |
| NT2RI2002316 | 0.669 | 0 | 0 | 0 | 0 | 3.592 | 0 |
| NT2RI2002391 | 0 | 0 | 0 | 0 | 0 | 1.817 | 0 |
| NT2RI2002540 | 0 | 6.399 | 0 | 0 | 0 | 0 | 0 |
| NT2RI2002654 | 1.470 | 0 | 0 | 0 | 0 | 0.658 | 0 |
| NT2RI2002847 | 1.047 | 0 | 0 | 0 | 0 | 4.218 | 0 |
| NT2RI2002865 | 0 | 0 | 0 | 0 | 0 | 7.595 | 0 |
| NT2RI2002958 | 0 | 21.383 | 0 | 0 | 0 | 1.078 | 0 |
| NT2RI2003011 | 0 | 0 | 0 | 71.453 | 0 | 0 | 0 |
| NT2RI2003019 | 8.722 | 0 | 0 | 0 | 0 | 0 | 0 |
| NT2RI2003051 | 18.780 | 0 | 0 | 0 | 0 | 0 | 0 |
| NT2RI2003304 | 2.407 | 0 | 0 | 0 | 0 | 1.470 | 0 |
| NT2RI2003317 | 1.314 | 0 | 0 | 0 | 0 | 0 | 0 |
| NT2RI2003338 | 0.844 | 7.496 | 0 | 0 | 0 | 1.134 | 0 |
| NT2RI2003344 | 3.248 | 0 | 0 | 0 | 0 | 4.363 | 0 |
| NT2RI2003407 | 4.037 | 0 | 0 | 0 | 0 | 0 | 25.993 |
| NT2RI2003420 | 2.568 | 0 | 0 | 0 | 0 | 1.725 | 0 |
| NT2RI2003678 | 7.938 | 0 | 0 | 0 | 0 | 2.133 | 0 |
| NT2RI2003695 | 0.657 | 2.915 | 0 | 0 | 0 | 0.882 | 0 |
| NT2RI2003738 | 0.438 | 0 | 2.405 | 0 | 0 | 1.372 | 3.757 |
| NT2RI2003921 | 0.791 | 0 | 0 | 0 | 0 | 0 | 0 |
| NT2RI2004059 | 5.682 | 0 | 0 | 0 | 0 | 0 | 0 |
| NT2RI2004304 | 17.362 | 0 | 0 | 0 | 0 | 4.665 | 0 |
| NT2RI2004535 | 0 | 0 | 0 | 0 | 0 | 2.144 | 0 |
| NT2RI2004608 | 3.085 | 0 | 0 | 0 | 0 | 0 | 0 |
| NT2RI2004884 | 0.644 | 0 | 0 | 0 | 0 | 0 | 0 |
| NT2RI2005061 | 0 | 0 | 0 | 0 | 0 | 4.886 | 0 |
| NT2RI2005116 | 1.066 | 0 | 0 | 0 | 0 | 0 | 0 |
| NT2RI2005150 | 0 | 2.551 | 0 | 0 | 0 | 0.772 | 0 |
| NT2RI2005335 | 3.228 | 2.866 | 0 | 0 | 0 | 3.469 | 0 |
| NT2RI2005353 | 0.961 | 4.264 | 7.919 | 0 | 0 | 0 | 0 |
| NT2RI2005405 | 0.234 | 1.039 | 0 | 0 | 12.229 | 0 | 0 |
| NT2RI2005473 | 0 | 0 | 0 | 0 | 71.141 | 0 | 0 |
| NT2RI2005647 | 0 | 0 | 0 | 0 | 0 | 0.741 | 0 |
| NT2RI2005710 | 0 | 0 | 0 | 0 | 0 | 5.847 | 0 |
| NT2RI2005713 | 1.233 | 0 | 0 | 0 | 0 | 0 | 0 |
| NT2RI2005723 | 2.092 | 0 | 0 | 0 | 0 | 0.562 | 10.777 |
| NT2RI2005811 | 1.277 | 5.671 | 0 | 0 | 0 | 0 | 0 |
| NT2RI2005814 | 0 | 0 | 0 | 0 | 0 | 1.459 | 0 |
| NT2RI2005818 | 2.124 | 0 | 0 | 0 | 0 | 0 | 0 |
| NT2RI2006070 | 8.569 | 0 | 0 | 42.530 | 0 | 0 | 0 |
| NT2RI2006072 | 3.828 | 0 | 0 | 0 | 0 | 0 | 0 |
| NT2RI2006271 | 0 | 10.339 | 0 | 0 | 0 | 0 | 0 |
| NT2RI2006412 | 0 | 3.034 | 0 | 0 | 0 | 0.918 | 0 |
| NT2RI2006565 | 0 | 0 | 0 | 0 | 0 | 13.386 | 0 |
| NT2RI2006640 | 0 | 23.265 | 0 | 0 | 0 | 0 | 0 |
| NT2RI2006682 | 2.052 | 0 | 0 | 0 | 0 | 5.513 | 0 |
| NT2RI2006703 | 13.514 | 0 | 0 | 0 | 0 | 0 | 0 |
| NT2RI2006853 | 0 | 0 | 0 | 0 | 0 | 5.025 | 0 |
| NT2RI2006856 | 1.172 | 0 | 0 | 0 | 0 | 0 | 0 |
| NT2RI2006943 | 0 | 4.502 | 0 | 0 | 0 | 0 | 0 |
| NT2RI2007054 | 1.083 | 4.806 | 0 | 0 | 0 | 0 | 0 |
| NT2RI2007084 | 0.907 | 0 | 0 | 0 | 0 | 0 | 0 |
| NT2RI2007096 | 4.323 | 0 | 0 | 0 | 0 | 0 | 0 |
| NT2RI2007133 | 0.327 | 0 | 2.697 | 0 | 0 | 0.879 | 0 |
| NT2RI2007277 | 0 | 3.629 | 0 | 0 | 0 | 1.098 | 0 |
| NT2RI2007377 | 0.977 | 8.671 | 0 | 0 | 0 | 0 | 0 |
| NT2RI2007384 | 0 | 0 | 0 | 0 | 0 | 0.485 | 0 |
| NT2RI2007445 | 1.495 | 0 | 0 | 0 | 0 | 0 | 0 |
| NT2RI2007468 | 6.968 | 0 | 0 | 0 | 0 | 0 | 0 |
| NT2RI2007572 | 0.623 | 0 | 5.137 | 0 | 0 | 0.837 | 0 |
| NT2RI2007589 | 0 | 3.953 | 0 | 0 | 0 | 0 | 0 |
| NT2RI2007629 | 0 | 7.462 | 0 | 0 | 0 | 0 | 0 |
| NT2RI2007723 | 2.950 | 0 | 0 | 0 | 0 | 0 | 0 |
| NT2RI2007884 | 2.117 | 0 | 0 | 0 | 0 | 0 | 0 |
| NT2RI2007987 | 4.504 | 6.665 | 0 | 0 | 0 | 2.017 | 0 |
| NT2RI2008045 | 0 | 0 | 0 | 0 | 0 | 2.609 | 0 |
| NT2RI2008141 | 0 | 0 | 0 | 0 | 0 | 4.790 | 0 |
| NT2RI2008336 | 0 | 1.483 | 0 | 0 | 8.726 | 0.897 | 0 |
| NT2RI2008396 | 2.205 | 0 | 0 | 0 | 0 | 0.494 | 0 |
| NT2RI2008526 | 1.279 | 0 | 0 | 0 | 0 | 0 | 0 |
| NT2RI2008566 | 7.843 | 0 | 0 | 0 | 0 | 0 | 0 |
| NT2RI2008714 | 0 | 0 | 0 | 0 | 0 | 16.436 | 0 |
| NT2RI2008724 | 3.063 | 0 | 12.626 | 0 | 0 | 0 | 0 |
| NT2RI2008791 | 0 | 0 | 0 | 0 | 0 | 1.307 | 0 |
| NT2RI2008808 | 3.780 | 0 | 0 | 0 | 0 | 5.078 | 0 |
| NT2RI2008942 | 1.749 | 0 | 0 | 0 | 0 | 11.746 | 0 |
| NT2RI2009037 | 2.812 | 0 | 0 | 0 | 18.363 | 1.889 | 0 |
| NT2RI2009065 | 1.558 | 1.729 | 0 | 0 | 0 | 0.523 | 0 |
| NT2RI2009066 | 0 | 0 | 0 | 0 | 0 | 6.140 | 0 |
| NT2RI2009083 | 8.550 | 0 | 0 | 0 | 0 | 0 | 0 |
| NT2RI2009173 | 0.930 | 0 | 0 | 0 | 0 | 0 | 0 |
| NT2RI2009194 | 0 | 3.638 | 0 | 0 | 0 | 0 | 0 |
| NT2RI2009239 | 3.496 | 0 | 0 | 0 | 0 | 0 | 0 |
| NT2RI2009259 | 9.759 | 0 | 0 | 0 | 0 | 0 | 0 |
| NT2RI2009269 | 0 | 7.316 | 0 | 0 | 0 | 0 | 0 |
| NT2RI2009406 | 0 | 0 | 0 | 0 | 0 | 3.151 | 0 |
| NT2RI2009583 | 0 | 0.828 | 4.615 | 13.890 | 0 | 0.752 | 0 |
| NT2RI2010283 | 0 | 0 | 50.879 | 0 | 0 | 0 | 0 |
| NT2RI2011450 | 0 | 0 | 0 | 0 | 0 | 28.449 | 0 |
| NT2RI2012726 | 3.868 | 0 | 0 | 0 | 0 | 0 | 0 |
| NT2RI2015342 | 0 | 5.569 | 0 | 0 | 0 | 0 | 0 |
| NT2RI2015533 | 1.296 | 11.506 | 0 | 0 | 0 | 3.482 | 0 |
| NT2RI2018448 | 0 | 0 | 0 | 0 | 0 | 14.106 | 0 |
| NT2RI2024008 | 0 | 0 | 0 | 0 | 0 | 38.134 | 0 |
| NT2RI2024313 | 12.217 | 0 | 0 | 0 | 0 | 0 | 0 |
| NT2RI2027224 | 0 | 0 | 0 | 0 | 0 | 38.134 | 0 |
| NT2RI3000174 | 0 | 0 | 0 | 0 | 0 | 38.134 | 0 |
| NT2RI3000423 | 8.019 | 0 | 0 | 0 | 0 | 48.475 | 0 |
| NT2RI3001445 | 5.281 | 0 | 0 | 0 | 0 | 0 | 0 |
| NT2RI3003738 | 15.123 | 0 | 0 | 0 | 0 | 0 | 0 |
| NT2RI3005923 | 0 | 0 | 0 | 0 | 0 | 21.835 | 0 |
| NT2RI3006284 | 0 | 0 | 0 | 0 | 0 | 13.906 | 0 |
| NT2RI3006673 | 0 | 0 | 0 | 0 | 0 | 11.178 | 0 |
| NT2RI3007291 | 0 | 0 | 0 | 0 | 0 | 14.020 | 0 |
| NT2RI3007543 | 1.415 | 0 | 0 | 0 | 0 | 2.851 | 0 |
| NT2RI3009480 | 31.453 | 0 | 0 | 0 | 0 | 0 | 0 |
| NT2RP6000017 | 2.495 | 0 | 0 | 0 | 0 | 1.676 | 0 |
| NT2RP6000039 | 1.495 | 0 | 0 | 0 | 0 | 0 | 0 |
| NT2RP6000072 | 12.126 | 5.981 | 0 | 0 | 0 | 0 | 0 |
| NT2RP6000078 | 32.080 | 1.331 | 2.472 | 0 | 0 | 0.805 | 7.722 |
| NT2RP6000085 | 0 | 0 | 0 | 0 | 0 | 10.361 | 0 |
| NT2RP6000110 | 0 | 0 | 0 | 0 | 0 | 6.135 | 0 |
| NT2RP6000123 | 0.979 | 1.449 | 0 | 0 | 0 | 0.877 | 0 |
| NT2RP7000069 | 8.426 | 0 | 0 | 0 | 0 | 7.546 | 0 |
| NT2RP7000076 | 2.541 | 6.768 | 0 | 0 | 0 | 1.365 | 0 |
| NT2RP7000086 | 0 | 0 | 0 | 0 | 0 | 14.324 | 0 |
| NT2RP7000112 | 13.393 | 0 | 0 | 0 | 0 | 0 | 0 |
| NT2RP7000173 | 0.464 | 0 | 0 | 0 | 0 | 2.491 | 0 |
| NT2RP7000238 | 6.476 | 0 | 0 | 0 | 0 | 1.450 | 0 |
| NT2RP7000259 | 0 | 0 | 0 | 0 | 0 | 14.979 | 0 |
| NT2RP7000271 | 5.058 | 0 | 0 | 0 | 0 | 3.397 | 0 |
| NT2RP7000477 | 0 | 0 | 0 | 0 | 0 | 2.216 | 0 |
| NT2RP7000600 | 5.543 | 0 | 0 | 0 | 0 | 4.964 | 0 |
| NT2RP7000906 | 0 | 0 | 0 | 0 | 0 | 7.306 | 0 |
| NT2RP7001166 | 3.118 | 2.768 | 0 | 0 | 0 | 1.675 | 0 |
| NT2RP7001231 | 3.444 | 0 | 0 | 0 | 0 | 9.252 | 0 |
| NT2RP7001283 | 0.469 | 0 | 0 | 0 | 0 | 1.891 | 0 |
| NT2RP7001335 | 0 | 15.467 | 0 | 0 | 0 | 0 | 0 |
| NT2RP7001591 | 2.168 | 0 | 0 | 0 | 0 | 0 | 0 |
| NT2RP7001962 | 0 | 0 | 0 | 0 | 0 | 2.976 | 0 |
| NT2RP7002028 | 3.351 | 7.439 | 0 | 0 | 0 | 2.251 | 0 |
| NT2RP7002282 | 0 | 0 | 0 | 0 | 0 | 17.201 | 0 |
| NT2RP7002376 | 20.234 | 0 | 0 | 0 | 0 | 0 | 0 |
| NT2RP7002379 | 1.679 | 1.863 | 0 | 0 | 0 | 1.128 | 0 |
| NT2RP7002449 | 0 | 7.478 | 0 | 0 | 0 | 2.263 | 0 |
| NT2RP7002450 | 0 | 0 | 0 | 0 | 0 | 2.990 | 0 |
| NT2RP7002619 | 0.909 | 0 | 0 | 0 | 0 | 0 | 0 |
| NT2RP7002802 | 3.047 | 0 | 0 | 0 | 0 | 0 | 0 |
| NT2RP7002829 | 0 | 0 | 0 | 0 | 0 | 9.794 | 0 |
| NT2RP7002875 | 1.539 | 3.416 | 0 | 0 | 0 | 0 | 0 |
| NT2RP7003203 | 1.540 | 0 | 0 | 0 | 0 | 0 | 0 |
| NT2RP7003319 | 0.761 | 0 | 0 | 0 | 0 | 6.132 | 0 |
| NT2RP7003511 | 24.799 | 0 | 0 | 0 | 0 | 0 | 0 |
| NT2RP7003629 | 9.435 | 0 | 0 | 0 | 0 | 12.674 | 0 |
| NT2RP7003632 | 2.716 | 0 | 0 | 0 | 0 | 0 | 0 |
| NT2RP7003647 | 3.925 | 0 | 0 | 0 | 0 | 10.546 | 0 |
| NT2RP7003688 | 4.926 | 0 | 0 | 0 | 0 | 4.963 | 0 |
| NT2RP7003960 | 0 | 0 | 0 | 0 | 0 | 1.856 | 0 |
| NT2RP7004114 | 4.650 | 0 | 0 | 0 | 0 | 6.246 | 0 |
| NT2RP7004123 | 1.600 | 0 | 0 | 0 | 0 | 2.149 | 0 |
| NT2RP7004173 | 0.637 | 0 | 0 | 0 | 0 | 2.566 | 0 |
| NT2RP7004196 | 1.093 | 0 | 0 | 0 | 0 | 0 | 0 |
| NT2RP7004204 | 1.422 | 0 | 0 | 0 | 0 | 0 | 0 |
| NT2RP7004260 | 21.394 | 0 | 0 | 0 | 0 | 0 | 0 |
| NT2RP7004348 | 5.653 | 0 | 0 | 0 | 0 | 2.531 | 0 |
| NT2RP7004352 | 5.665 | 0 | 0 | 0 | 0 | 3.805 | 0 |
| NT2RP7004373 | 5.888 | 0 | 8.092 | 0 | 0 | 7.910 | 0 |
| NT2RP7004428 | 5.624 | 0 | 0 | 0 | 0 | 3.778 | 0 |
| NT2RP7004641 | 21.394 | 0 | 0 | 0 | 0 | 0 | 0 |
| NT2RP7004656 | 0 | 0 | 0 | 0 | 0 | 48.174 | 0 |
| NT2RP7004687 | 0 | 0 | 0 | 0 | 0 | 42.237 | 0 |
| NT2RP7004728 | 2.621 | 0 | 0 | 0 | 0 | 3.521 | 0 |
| NT2RP7004751 | 0 | 1.733 | 0 | 0 | 0 | 0.524 | 0 |
| NT2RP7004884 | 0 | 10.162 | 0 | 0 | 0 | 3.075 | 0 |
| NT2RP7004911 | 2.939 | 6.524 | 0 | 0 | 0 | 0 | 0 |
| NT2RP7004925 | 4.211 | 0 | 0 | 0 | 0 | 5.657 | 0 |
| NT2RP7004946 | 2.871 | 3.187 | 0 | 0 | 0 | 0.964 | 0 |
| NT2RP7004961 | 0 | 0 | 0 | 0 | 0 | 18.856 | 0 |
| NT2RP7005493 | 0 | 0 | 0 | 0 | 0 | 27.110 | 0 |
| NT2RP7005529 | 0 | 0 | 0 | 0 | 0 | 8.270 | 0 |
| NT2RP7005675 | 0 | 0 | 0 | 0 | 0 | 22.803 | 0 |
| NT2RP7005750 | 5.967 | 0 | 0 | 0 | 0 | 0 | 0 |
| NT2RP7006141 | 3.079 | 0 | 15.231 | 15.282 | 0 | 0 | 0 |
| NT2RP7006188 | 4.342 | 0 | 0 | 0 | 0 | 2.916 | 0 |
| NT2RP7006223 | 0 | 0 | 0 | 0 | 0 | 9.466 | 0 |
| NT2RP7006490 | 0 | 0 | 0 | 0 | 0 | 2.404 | 0 |
| NT2RP7006539 | 0 | 0 | 0 | 0 | 0 | 6.518 | 0 |
| NT2RP7006601 | 0 | 0 | 0 | 0 | 0 | 31.730 | 0 |
| NT2RP7006619 | 0 | 8.170 | 0 | 0 | 0 | 0 | 0 |
| NT2RP7006701 | 4.192 | 0 | 0 | 0 | 0 | 0 | 0 |
| NT2RP7006717 | 2.194 | 0 | 0 | 0 | 0 | 3.685 | 0 |
| NT2RP7006886 | 0 | 0 | 0 | 0 | 0 | 10.654 | 0 |
| NT2RP7006986 | 0 | 27.811 | 0 | 0 | 0 | 0 | 0 |
| NT2RP7006995 | 0 | 0 | 0 | 0 | 0 | 48.174 | 0 |
| NT2RP7007154 | 0 | 2.111 | 0 | 0 | 0 | 1.277 | 0 |
| NT2RP7007177 | 5.678 | 0 | 0 | 0 | 0 | 0 | 0 |
| NT2RP7007252 | 5.726 | 0 | 0 | 0 | 0 | 0 | 0 |
| NT2RP7007381 | 0 | 0 | 0 | 0 | 0 | 17.566 | 0 |
| NT2RP7007387 | 1.712 | 0 | 0 | 0 | 0 | 4.598 | 0 |
| NT2RP7007406 | 25.705 | 0 | 0 | 0 | 0 | 0 | 0 |
| NT2RP7007530 | 3.656 | 0 | 0 | 0 | 0 | 4.911 | 0 |
| NT2RP7007537 | 2.051 | 0 | 0 | 0 | 0 | 5.509 | 0 |
| NT2RP7007580 | 0 | 0 | 0 | 0 | 0 | 26.772 | 0 |
| NT2RP7007610 | 2.413 | 21.426 | 0 | 0 | 0 | 3.242 | 0 |
| NT2RP7007766 | 25.705 | 0 | 0 | 0 | 0 | 0 | 0 |
| NT2RP7007975 | 0 | 0 | 0 | 0 | 0 | 1.529 | 29.307 |
| NT2RP7008015 | 1.683 | 0 | 0 | 0 | 0 | 1.130 | 0 |
| NT2RP7008161 | 5.235 | 0 | 0 | 0 | 0 | 0 | 0 |
| NT2RP7008167 | 1.514 | 0 | 0 | 0 | 0 | 6.100 | 0 |
| NT2RP7008315 | 12.515 | 0 | 0 | 0 | 0 | 0 | 0 |
| NT2RP7008543 | 2.294 | 0 | 0 | 0 | 0 | 6.163 | 0 |
| NT2RP7009087 | 6.077 | 0 | 0 | 0 | 0 | 5.442 | 0 |
| NT2RP7009097 | 0 | 13.310 | 0 | 0 | 0 | 0 | 0 |
| NT2RP7009108 | 0 | 3.290 | 0 | 0 | 0 | 0 | 0 |
| NT2RP7009147 | 1.328 | 0 | 0 | 0 | 0 | 7.137 | 0 |
| NT2RP7009149 | 0 | 0 | 0 | 0 | 0 | 1.340 | 0 |
| NT2RP7009259 | 0.468 | 0 | 0 | 0 | 0 | 2.517 | 0 |
| NT2RP7009394 | 0 | 0 | 0 | 0 | 0 | 2.709 | 0 |
| NT2RP7009466 | 25.705 | 0 | 0 | 0 | 0 | 0 | 0 |
| NT2RP7009481 | 0 | 0 | 0 | 0 | 0 | 9.175 | 0 |
| NT2RP7009482 | 1.546 | 0 | 0 | 0 | 0 | 1.557 | 0 |
| NT2RP7009498 | 0.840 | 0 | 0 | 0 | 0 | 0 | 0 |
| NT2RP7010109 | 13.103 | 0 | 0 | 0 | 0 | 0 | 0 |
| NT2RP7013573 | 6.009 | 0 | 0 | 0 | 0 | 0 | 0 |
| NT2RP7013764 | 0 | 0 | 0 | 0 | 0 | 12.717 | 0 |
| NT2RP7013795 | 0 | 0 | 0 | 89.568 | 0 | 0 | 0 |
| NT2RP7014005 | 4.851 | 0 | 0 | 0 | 0 | 6.517 | 0 |
| NT2RP7014583 | 0 | 60.566 | 0 | 0 | 0 | 0 | 0 |
| NT2RP7014910 | 1.994 | 0 | 0 | 0 | 0 | 1.339 | 0 |
| NT2RP8000435 | 11.126 | 0 | 0 | 0 | 0 | 0 | 0 |
| NT2RP8005546 | 40.897 | 0 | 0 | 0 | 0 | 0 | 0 |
| NT2RP8007920 | 1.649 | 0 | 0 | 0 | 0 | 2.216 | 0 |
| NT2RP8008057 | 0 | 0 | 0 | 0 | 0 | 31.730 | 0 |
| NTONG1000033 | 6.130 | 0 | 0 | 0 | 0 | 5.489 | 0 |
| NTONG1000075 | 5.556 | 0 | 0 | 0 | 0 | 0 | 0 |
| NTONG1000098 | 3.188 | 0 | 0 | 0 | 0 | 4.282 | 0 |
| NTONG1000130 | 0 | 0 | 0 | 0 | 0 | 13.193 | 0 |
| NTONG1000182 | 0 | 0 | 0 | 0 | 0 | 1.387 | 0 |
| NTONG1000214 | 9.258 | 0 | 0 | 0 | 0 | 0 | 0 |
| NTONG1000246 | 2.608 | 5.788 | 0 | 0 | 0 | 1.751 | 0 |
| NTONG1000264 | 1.625 | 7.216 | 0 | 0 | 0 | 2.184 | 0 |
| NTONG2000265 | 2.133 | 0 | 0 | 0 | 0 | 1.910 | 0 |
| NTONG2000800 | 0 | 0 | 0 | 0 | 0 | 0.593 | 0 |
| NTONG2000876 | 0 | 5.154 | 0 | 0 | 0 | 0 | 0 |
| NTONG2000977 | 0 | 0 | 0 | 0 | 0 | 1.101 | 21.112 |
| NTONG2000985 | 0 | 0 | 0 | 0 | 0 | 2.088 | 0 |
| NTONG2001137 | 0.954 | 0 | 0 | 0 | 0 | 1.282 | 0 |
| NTONG2001550 | 2.902 | 0 | 0 | 0 | 0 | 3.899 | 0 |
| NTONG2001567 | 0 | 0 | 0 | 0 | 0 | 0.687 | 6.585 |
| NTONG2001612 | 0 | 4.155 | 0 | 0 | 0 | 0 | 0 |
| NTONG2001762 | 1.504 | 0 | 0 | 0 | 0 | 0 | 0 |
| NTONG2002582 | 0 | 0 | 0 | 0 | 0 | 16.852 | 0 |
| NTONG2003454 | 2.486 | 0 | 0 | 0 | 0 | 3.339 | 0 |
| NTONG2003928 | 0 | 43.126 | 0 | 0 | 0 | 0 | 0 |
| NTONG2004095 | 22.967 | 0 | 0 | 0 | 0 | 0 | 0 |
| NTONG2004308 | 0 | 0 | 0 | 0 | 0 | 13.193 | 0 |
| NTONG2004614 | 4.938 | 0 | 0 | 0 | 0 | 0 | 0 |
| NTONG2004918 | 5.400 | 0 | 0 | 0 | 0 | 0 | 0 |
| NTONG2005373 | 0 | 0 | 0 | 0 | 0 | 6.601 | 0 |
| NTONG2005391 | 0 | 0 | 0 | 0 | 0 | 2.611 | 0 |
| NTONG2005520 | 0 | 0 | 0 | 0 | 0 | 1.491 | 0 |
| NTONG2005801 | 1.486 | 0 | 0 | 0 | 0 | 1.996 | 0 |
| NTONG2005969 | 0 | 11.891 | 0 | 0 | 0 | 0 | 0 |
| NTONG2006484 | 0 | 0 | 0 | 0 | 0 | 4.842 | 0 |
| NTONG2008143 | 9.060 | 0 | 0 | 0 | 0 | 0 | 0 |
| NTONG2008483 | 0 | 0 | 0 | 0 | 0 | 1.790 | 0 |
| NTONG2008522 | 0 | 0 | 0 | 0 | 0 | 4.390 | 0 |
| NTONG2008944 | 4.869 | 0 | 0 | 0 | 0 | 3.271 | 0 |
| NTONG2009060 | 0 | 0 | 0 | 0 | 60.694 | 0 | 0 |
| NTONG2009576 | 0 | 0 | 0 | 0 | 0 | 7.959 | 0 |
| OCBBF1000080 | 0 | 0 | 0 | 0 | 0 | 21.417 | 0 |
| OCBBF1000086 | 0.638 | 0 | 0 | 0 | 0 | 0 | 0 |
| OCBBF1000104 | 0 | 0 | 0 | 0 | 0 | 11.536 | 0 |
| OCBBF1000146 | 0 | 6.386 | 0 | 0 | 0 | 0 | 0 |
| OCBBF1000315 | 14.591 | 0 | 0 | 0 | 0 | 0 | 0 |
| OCBBF2000013 | 3.245 | 0 | 0 | 0 | 0 | 0 | 0 |
| OCBBF2000015 | 1.979 | 0 | 0 | 0 | 0 | 4.431 | 0 |
| OCBBF2000126 | 0 | 14.387 | 0 | 0 | 0 | 0 | 0 |
| OCBBF2000178 | 0 | 4.036 | 0 | 0 | 0 | 0 | 0 |
| OCBBF2000231 | 0 | 14.500 | 0 | 0 | 0 | 0 | 0 |
| OCBBF2000522 | 0 | 0 | 0 | 0 | 0 | 2.832 | 0 |
| OCBBF2000677 | 13.583 | 0 | 0 | 0 | 0 | 0 | 0 |
| OCBBF2000831 | 0 | 0 | 0 | 0 | 0 | 5.393 | 0 |
| OCBBF2000846 | 0.610 | 0 | 0 | 0 | 0 | 2.459 | 0 |
| OCBBF2000982 | 0.727 | 0 | 0 | 0 | 0 | 0.977 | 0 |
| OCBBF2000986 | 1.607 | 0 | 0 | 0 | 0 | 0 | 0 |
| OCBBF2001166 | 0 | 0 | 0 | 0 | 0 | 18.418 | 0 |
| OCBBF2001196 | 13.205 | 0 | 0 | 0 | 0 | 0 | 0 |
| OCBBF2001307 | 0 | 6.157 | 0 | 0 | 0 | 0 | 0 |
| OCBBF2001492 | 1.716 | 2.540 | 0 | 0 | 0 | 1.537 | 0 |
| OCBBF2001528 | 0 | 0 | 0 | 0 | 0 | 37.138 | 0 |
| OCBBF2001639 | 3.921 | 17.407 | 0 | 0 | 0 | 0 | 0 |
| OCBBF2001687 | 0 | 0 | 0 | 0 | 0 | 4.309 | 0 |
| OCBBF2001706 | 40.164 | 0 | 0 | 0 | 0 | 0 | 0 |
| OCBBF2001983 | 0 | 26.741 | 0 | 0 | 0 | 0 | 0 |
| OCBBF2002015 | 57.664 | 0 | 0 | 0 | 0 | 0 | 0 |
| OCBBF2002086 | 2.686 | 0 | 0 | 0 | 0 | 0 | 0 |
| OCBBF2002290 | 0 | 37.332 | 0 | 0 | 0 | 0 | 0 |
| OCBBF2002656 | 1.379 | 0 | 11.372 | 0 | 0 | 1.853 | 35.524 |
| OCBBF2003052 | 0 | 14.060 | 0 | 0 | 0 | 0 | 0 |
| OCBBF2003518 | 0 | 0 | 0 | 0 | 0 | 1.155 | 0 |
| OCBBF2003543 | 40.164 | 0 | 0 | 0 | 0 | 0 | 0 |
| OCBBF2003744 | 0 | 0 | 0 | 0 | 0 | 47.415 | 0 |
| OCBBF2003819 | 0 | 0 | 0 | 0 | 0 | 23.601 | 0 |
| OCBBF2003925 | 0 | 3.084 | 0 | 0 | 0 | 0 | 0 |
| OCBBF2004104 | 0 | 10.140 | 0 | 0 | 0 | 0 | 0 |
| OCBBF2004273 | 0 | 3.318 | 0 | 0 | 0 | 1.004 | 0 |
| OCBBF2006660 | 12.586 | 0 | 0 | 0 | 0 | 0 | 0 |
| OCBBF2006849 | 6.741 | 0 | 0 | 0 | 0 | 4.528 | 0 |
| OCBBF2007028 | 1.151 | 0 | 0 | 0 | 0 | 0 | 0 |
| OCBBF2007121 | 0 | 0 | 0 | 0 | 0 | 47.415 | 0 |
| OCBBF2007224 | 0 | 0 | 0 | 0 | 0 | 10.576 | 0 |
| OCBBF2007354 | 0.838 | 0 | 6.907 | 0 | 0 | 3.376 | 0 |
| OCBBF2007414 | 40.164 | 0 | 0 | 0 | 0 | 0 | 0 |
| OCBBF2007415 | 0.668 | 11.856 | 0 | 0 | 0 | 0.897 | 0 |
| OCBBF2007829 | 0.458 | 0 | 0 | 0 | 0 | 0.615 | 0 |
| OCBBF2007892 | 10.657 | 0 | 0 | 0 | 0 | 0 | 0 |
| OCBBF2008005 | 0 | 11.755 | 0 | 0 | 0 | 0 | 0 |
| OCBBF2008041 | 1.743 | 0 | 0 | 0 | 0 | 0 | 0 |
| OCBBF2008822 | 0 | 0 | 16.388 | 0 | 0 | 0 | 0 |
| OCBBF2009242 | 11.805 | 0 | 0 | 0 | 0 | 3.172 | 0 |
| OCBBF2009301 | 0 | 0 | 0 | 0 | 0 | 4.644 | 0 |
| OCBBF2009772 | 0.982 | 8.718 | 0 | 0 | 0 | 0 | 0 |
| OCBBF2009836 | 0 | 15.168 | 0 | 0 | 0 | 0 | 0 |
| OCBBF2010140 | 27.107 | 0 | 0 | 0 | 0 | 0 | 0 |
| OCBBF2010281 | 0 | 0 | 0 | 0 | 0 | 47.415 | 0 |
| OCBBF2010557 | 0 | 0 | 0 | 0 | 0 | 20.524 | 0 |
| OCBBF2010830 | 0 | 0 | 6.049 | 0 | 0 | 0 | 0 |
| OCBBF2010841 | 0 | 0 | 0 | 0 | 0 | 13.925 | 0 |
| OCBBF2011021 | 0 | 0 | 0 | 0 | 0 | 4.857 | 0 |
| OCBBF2011759 | 0.779 | 0 | 0 | 0 | 0 | 5.234 | 0 |
| OCBBF2011767 | 0 | 0 | 0 | 0 | 0 | 33.313 | 0 |
| OCBBF2011887 | 0 | 17.408 | 0 | 0 | 0 | 5.268 | 0 |
| OCBBF2012001 | 0 | 0 | 0 | 0 | 0 | 27.891 | 0 |
| OCBBF2012436 | 40.164 | 0 | 0 | 0 | 0 | 0 | 0 |
| OCBBF2012553 | 0.978 | 4.341 | 0 | 0 | 0 | 0 | 0 |
| OCBBF2012714 | 0 | 0 | 0 | 0 | 0 | 6.502 | 0 |
| OCBBF2012821 | 0 | 0 | 0 | 0 | 0 | 2.803 | 0 |
| OCBBF2013011 | 0 | 27.949 | 0 | 0 | 0 | 0 | 0 |
| OCBBF2013123 | 0 | 0 | 0 | 0 | 0 | 25.558 | 0 |
| OCBBF2013149 | 0 | 34.448 | 0 | 0 | 0 | 0 | 0 |
| OCBBF2013366 | 2.251 | 0 | 0 | 0 | 0 | 0 | 0 |
| OCBBF2013843 | 0.446 | 1.978 | 0 | 0 | 0 | 0.599 | 0 |
| OCBBF2014052 | 0 | 0 | 0 | 0 | 0 | 47.415 | 0 |
| OCBBF2014064 | 0 | 5.056 | 0 | 0 | 0 | 1.530 | 0 |
| OCBBF2016612 | 0 | 0 | 0 | 0 | 0 | 47.415 | 0 |
| OCBBF2016729 | 0 | 0 | 0 | 0 | 0 | 20.422 | 0 |
| OCBBF2017035 | 0 | 4.488 | 0 | 0 | 0 | 0 | 0 |
| OCBBF2017055 | 14.962 | 0 | 0 | 0 | 0 | 0 | 0 |
| OCBBF2017398 | 1.073 | 9.523 | 0 | 0 | 0 | 2.882 | 0 |
| OCBBF2017754 | 6.261 | 0 | 0 | 0 | 0 | 4.206 | 0 |
| OCBBF2017791 | 0 | 0 | 0 | 0 | 0 | 2.869 | 0 |
| OCBBF2017893 | 4.339 | 0 | 0 | 35.895 | 0 | 0 | 0 |
| OCBBF2018012 | 0 | 0 | 0 | 0 | 0 | 6.288 | 0 |
| OCBBF2018563 | 0 | 0 | 0 | 0 | 0 | 23.036 | 0 |
| OCBBF2018581 | 0 | 15.925 | 0 | 0 | 0 | 0 | 0 |
| OCBBF2018827 | 0 | 18.163 | 0 | 0 | 0 | 5.496 | 0 |
| OCBBF2018956 | 1.411 | 0 | 0 | 0 | 0 | 0 | 0 |
| OCBBF2019108 | 57.310 | 0 | 0 | 0 | 0 | 0 | 0 |
| OCBBF2020318 | 40.164 | 0 | 0 | 0 | 0 | 0 | 0 |
| OCBBF2020801 | 40.164 | 0 | 0 | 0 | 0 | 0 | 0 |
| OCBBF2024850 | 0 | 0 | 0 | 0 | 0 | 4.979 | 0 |
| OCBBF2028421 | 0 | 0 | 0 | 0 | 0 | 47.415 | 0 |
| OCBBF2031561 | 26.180 | 0 | 0 | 0 | 0 | 0 | 0 |
| OCBBF2035823 | 57.310 | 0 | 0 | 0 | 0 | 0 | 0 |
| OCBBF2036743 | 9.217 | 0 | 0 | 0 | 0 | 0 | 0 |
| OCBBF3000296 | 0 | 0 | 0 | 0 | 0 | 21.220 | 0 |
| OCBBF3000323 | 16.639 | 0 | 0 | 0 | 0 | 0 | 0 |
| OCBBF3001333 | 16.074 | 0 | 0 | 0 | 0 | 0 | 0 |
| OCBBF3004487 | 3.778 | 16.771 | 0 | 0 | 0 | 10.150 | 0 |
| PANCR1000091 | 0 | 1.114 | 0 | 0 | 0 | 0 | 0 |
| PEBLM1000066 | 3.952 | 0 | 0 | 0 | 0 | 5.308 | 0 |
| PEBLM1000144 | 5.001 | 0 | 0 | 0 | 0 | 0 | 0 |
| PEBLM2000030 | 6.138 | 0 | 0 | 0 | 0 | 28.858 | 0 |
| PEBLM2000147 | 5.319 | 4.722 | 0 | 0 | 0 | 2.858 | 0 |
| PEBLM2000222 | 0.484 | 2.150 | 0 | 0 | 0 | 0 | 12.475 |
| PEBLM2000308 | 6.612 | 7.338 | 0 | 0 | 0 | 0 | 0 |
| PEBLM2000326 | 13.579 | 0 | 0 | 0 | 0 | 0 | 0 |
| PEBLM2000338 | 7.936 | 0 | 0 | 0 | 0 | 0 | 0 |
| PEBLM2000395 | 0 | 6.738 | 0 | 0 | 0 | 1.020 | 0 |
| PEBLM2001072 | 1.512 | 20.138 | 0 | 0 | 0 | 2.031 | 0 |
| PEBLM2003914 | 7.087 | 0 | 0 | 0 | 0 | 7.140 | 0 |
| PEBLM2004452 | 1.125 | 0 | 0 | 0 | 0 | 7.559 | 0 |
| PEBLM2004666 | 3.098 | 0 | 0 | 0 | 0 | 0 | 0 |
| PEBLM2006036 | 9.986 | 0 | 0 | 0 | 0 | 0 | 0 |
| PEBLM2006709 | 0 | 0 | 0 | 0 | 0 | 6.232 | 0 |
| PEBLM2007188 | 7.279 | 0 | 0 | 0 | 0 | 0 | 0 |
| PEBLM2007296 | 4.326 | 0 | 0 | 0 | 0 | 0 | 0 |
| PEBLM2007447 | 9.986 | 0 | 0 | 0 | 0 | 0 | 0 |
| PEBLM2007774 | 0.502 | 0 | 0 | 0 | 6.560 | 0.337 | 0 |
| PERIC2000889 | 14.835 | 0 | 0 | 0 | 0 | 9.964 | 0 |
| PERIC2007068 | 1.287 | 0 | 0 | 0 | 0 | 8.642 | 0 |
| PLACE5000013 | 6.618 | 0 | 0 | 0 | 0 | 1.778 | 0 |
| PLACE5000068 | 0 | 23.246 | 0 | 0 | 0 | 7.034 | 0 |
| PLACE5000113 | 0 | 0 | 0 | 0 | 0 | 1.927 | 0 |
| PLACE5000159 | 0 | 0 | 0 | 0 | 0 | 5.091 | 0 |
| PLACE5000170 | 0 | 0 | 0 | 0 | 0 | 3.894 | 0 |
| PLACE6000145 | 0 | 0 | 0 | 0 | 0 | 1.161 | 0 |
| PLACE6000325 | 6.755 | 0 | 0 | 0 | 0 | 0 | 0 |
| PLACE6000379 | 4.566 | 1.961 | 0 | 0 | 3.847 | 0.791 | 0 |
| PLACE6000414 | 7.479 | 0 | 0 | 0 | 0 | 1.435 | 0 |
| PLACE6000463 | 6.506 | 0 | 0 | 0 | 0 | 0 | 0 |
| PLACE6000630 | 0 | 0 | 0 | 0 | 0 | 4.322 | 0 |
| PLACE6001064 | 0.957 | 0 | 0 | 0 | 0 | 5.784 | 0 |
| PLACE6001281 | 2.871 | 0 | 0 | 0 | 0 | 0 | 0 |
| PLACE6001443 | 1.638 | 14.542 | 0 | 0 | 0 | 0 | 0 |
| PLACE6002084 | 0.924 | 0 | 0 | 7.643 | 0 | 0.414 | 0 |
| PLACE6002102 | 1.178 | 0 | 0 | 0 | 0 | 0 | 0 |
| PLACE6002668 | 0 | 0 | 0 | 0 | 0 | 6.045 | 0 |
| PLACE6002960 | 1.851 | 0 | 0 | 0 | 0 | 6.216 | 0 |
| PLACE6003218 | 0 | 0 | 0 | 0 | 0 | 16.300 | 0 |
| PLACE6003383 | 0.995 | 0 | 0 | 0 | 0 | 5.348 | 0 |
| PLACE6003745 | 0 | 0 | 0 | 0 | 0 | 11.772 | 0 |
| PLACE6004005 | 0 | 7.309 | 0 | 0 | 0 | 0 | 0 |
| PLACE6004312 | 2.012 | 4.465 | 0 | 0 | 0 | 1.351 | 0 |
| PLACE6004397 | 1.533 | 6.804 | 0 | 0 | 0 | 0 | 0 |
| PLACE6005231 | 9.210 | 0 | 0 | 0 | 0 | 0 | 0 |
| PLACE6005294 | 0 | 0 | 0 | 0 | 0 | 13.488 | 0 |
| PLACE6005482 | 2.205 | 0 | 0 | 0 | 0 | 23.695 | 0 |
| PLACE6006042 | 4.539 | 0 | 0 | 0 | 0 | 0 | 0 |
| PLACE6006137 | 0 | 12.643 | 0 | 0 | 0 | 0 | 0 |
| PLACE6006186 | 0 | 0 | 0 | 0 | 0 | 8.102 | 0 |
| PLACE6006549 | 0 | 0 | 5.490 | 0 | 0 | 0.894 | 0 |
| PLACE6007239 | 0 | 0 | 0 | 0 | 0 | 4.618 | 0 |
| PLACE6008775 | 1.537 | 0 | 0 | 0 | 0 | 6.194 | 0 |
| PLACE6008793 | 0 | 5.869 | 0 | 0 | 0 | 0 | 0 |
| PLACE6009237 | 0 | 0 | 0 | 0 | 0 | 0 | 92.347 |
| PLACE6010077 | 0 | 0 | 5.986 | 0 | 0 | 1.951 | 0 |
| PLACE6010484 | 5.155 | 0 | 0 | 0 | 0 | 0 | 0 |
| PLACE6010848 | 6.823 | 0 | 0 | 0 | 0 | 9.166 | 0 |
| PLACE6011057 | 1.879 | 0 | 0 | 0 | 0 | 10.098 | 0 |
| PLACE6011970 | 21.626 | 0 | 0 | 0 | 0 | 0 | 0 |
| PLACE6012028 | 0 | 23.236 | 0 | 0 | 0 | 0 | 0 |
| PLACE6012936 | 31.905 | 0 | 0 | 0 | 0 | 0 | 0 |
| PLACE6013222 | 0 | 18.239 | 0 | 0 | 0 | 0 | 0 |
| PLACE6013232 | 0 | 0 | 79.437 | 0 | 0 | 0 | 0 |
| PLACE6013672 | 23.027 | 0 | 0 | 0 | 0 | 0 | 0 |
| PLACE6016254 | 15.235 | 0 | 0 | 0 | 0 | 20.465 | 0 |
| PLACE6016942 | 0 | 0 | 26.895 | 0 | 0 | 0 | 0 |
| PLACE6016984 | 15.403 | 0 | 0 | 0 | 0 | 0 | 0 |
| PLACE6018031 | 5.606 | 0 | 0 | 0 | 0 | 0 | 0 |
| PLACE6018487 | 0.556 | 0 | 0 | 0 | 0 | 0 | 0 |
| PLACE6018938 | 0 | 0 | 0 | 0 | 0 | 8.594 | 0 |
| PLACE6019195 | 0 | 0 | 0 | 0 | 0 | 9.505 | 0 |
| PLACE7001022 | 0 | 0 | 0 | 0 | 0 | 31.522 | 0 |
| PLACE7004103 | 1.078 | 0 | 0 | 0 | 0 | 7.243 | 0 |
| PLACE7005840 | 31.905 | 0 | 0 | 0 | 0 | 0 | 0 |
| PROST1000097 | 3.432 | 0 | 0 | 0 | 0 | 0.659 | 0 |
| PROST1000120 | 0 | 0 | 0 | 0 | 0 | 3.694 | 0 |
| PROST1000199 | 2.437 | 2.705 | 0 | 0 | 0 | 1.637 | 0 |
| PROST1000215 | 4.265 | 0 | 0 | 0 | 0 | 0 | 0 |
| PROST1000220 | 0 | 0 | 0 | 0 | 0 | 0.761 | 0 |
| PROST1000226 | 5.968 | 0 | 0 | 0 | 0 | 0 | 0 |
| PROST1000246 | 9.161 | 0 | 0 | 0 | 0 | 0 | 0 |
| PROST1000343 | 7.106 | 0 | 0 | 0 | 0 | 3.182 | 0 |
| PROST1000526 | 2.107 | 0 | 0 | 0 | 0 | 2.830 | 0 |
| PROST1000536 | 0 | 0 | 0 | 0 | 0 | 6.735 | 0 |
| PROST1000564 | 5.604 | 0 | 0 | 0 | 0 | 1.506 | 0 |
| PROST2000036 | 2.344 | 10.406 | 0 | 0 | 0 | 0 | 0 |
| PROST2000138 | 0.145 | 0.321 | 2.383 | 0 | 0 | 1.650 | 0 |
| PROST2000241 | 0 | 0 | 0 | 0 | 0 | 6.855 | 0 |
| PROST2000274 | 8.605 | 0 | 0 | 0 | 0 | 11.559 | 0 |
| PROST2000277 | 0 | 0 | 0 | 0 | 0 | 24.004 | 0 |
| PROST2000284 | 8.760 | 0 | 0 | 0 | 0 | 0 | 0 |
| PROST2000567 | 0 | 0 | 0 | 0 | 0 | 16.044 | 0 |
| PROST2000760 | 0 | 10.384 | 0 | 0 | 0 | 0 | 60.243 |
| PROST2000835 | 0 | 0 | 0 | 0 | 0 | 24.004 | 0 |
| PROST2001002 | 16.382 | 0 | 0 | 0 | 0 | 0 | 0 |
| PROST2001116 | 0 | 0 | 0 | 0 | 0 | 24.004 | 0 |
| PROST2001180 | 2.070 | 9.191 | 0 | 0 | 0 | 0 | 0 |
| PROST2001883 | 0 | 3.306 | 0 | 0 | 0 | 0 | 0 |
| PROST2001997 | 0 | 0 | 40.082 | 0 | 0 | 0 | 0 |
| PROST2002651 | 0 | 0 | 0 | 0 | 0 | 3.350 | 0 |
| PROST2002897 | 0 | 0 | 0 | 0 | 0 | 24.004 | 0 |
| PROST2002960 | 11.208 | 0 | 0 | 0 | 0 | 0 | 0 |
| PROST2003232 | 1.707 | 0 | 0 | 0 | 0 | 0 | 0 |
| PROST2003338 | 0.933 | 0 | 0 | 0 | 0 | 1.254 | 0 |
| PROST2003396 | 0 | 0 | 0 | 0 | 0 | 26.768 | 0 |
| PROST2003517 | 2.195 | 0 | 0 | 0 | 0 | 2.949 | 0 |
| PROST2003537 | 0 | 0 | 0 | 0 | 0 | 24.004 | 0 |
| PROST2003725 | 19.037 | 0 | 0 | 0 | 0 | 0 | 0 |
| PROST2004095 | 0.390 | 1.733 | 0 | 0 | 0 | 0.524 | 0 |
| PROST2004270 | 0.981 | 2.177 | 4.043 | 0 | 0 | 0.659 | 0 |
| PROST2004416 | 0.567 | 0 | 0 | 0 | 0 | 1.523 | 0 |
| PROST2004739 | 0 | 0 | 0 | 0 | 0 | 8.329 | 0 |
| PROST2004744 | 4.408 | 0 | 0 | 0 | 0 | 0 | 0 |
| PROST2005067 | 13.451 | 0 | 0 | 0 | 0 | 0 | 0 |
| PROST2005143 | 5.657 | 25.111 | 0 | 0 | 0 | 7.599 | 0 |
| PROST2005272 | 0 | 0 | 0 | 0 | 0 | 9.199 | 0 |
| PROST2005285 | 18.671 | 0 | 0 | 0 | 0 | 0 | 0 |
| PROST2005604 | 5.187 | 0 | 0 | 0 | 0 | 17.421 | 0 |
| PROST2005793 | 0 | 0 | 0 | 0 | 0 | 13.178 | 0 |
| PROST2006030 | 0 | 0 | 0 | 0 | 0 | 12.715 | 0 |
| PROST2006060 | 5.567 | 0 | 0 | 0 | 0 | 0 | 0 |
| PROST2006260 | 0 | 0 | 0 | 0 | 0 | 9.122 | 0 |
| PROST2006343 | 0 | 0 | 0 | 0 | 0 | 4.953 | 0 |
| PROST2006536 | 0 | 0 | 0 | 0 | 0 | 4.971 | 0 |
| PROST2006737 | 2.021 | 0 | 16.663 | 0 | 0 | 2.715 | 0 |
| PROST2007612 | 7.988 | 0 | 0 | 0 | 0 | 0 | 0 |
| PROST2008271 | 6.536 | 0 | 0 | 0 | 0 | 13.169 | 0 |
| PROST2008360 | 1.501 | 0 | 0 | 0 | 0 | 2.016 | 38.653 |
| PROST2008489 | 0.540 | 0 | 0 | 13.402 | 0 | 0 | 0 |
| PROST2008770 | 5.203 | 2.887 | 0 | 0 | 16.991 | 0.874 | 0 |
| PROST2010219 | 0 | 0 | 0 | 0 | 0 | 24.004 | 0 |
| PROST2010545 | 0 | 0 | 0 | 0 | 0 | 14.741 | 0 |
| PROST2010579 | 0 | 0 | 0 | 0 | 0 | 21.054 | 0 |
| PROST2010782 | 0.203 | 0 | 0 | 0 | 0 | 0 | 5.223 |
| PROST2011577 | 0 | 0 | 0 | 0 | 0 | 2.305 | 0 |
| PROST2012007 | 0.946 | 0 | 0 | 0 | 0 | 0 | 0 |
| PROST2012448 | 0 | 0 | 0 | 0 | 0 | 9.139 | 0 |
| PROST2012504 | 19.037 | 0 | 0 | 0 | 0 | 0 | 0 |
| PROST2012745 | 0 | 0 | 0 | 0 | 0 | 4.332 | 0 |
| PROST2013121 | 8.969 | 0 | 0 | 0 | 0 | 12.048 | 0 |
| PROST2015287 | 0 | 0 | 0 | 0 | 0 | 24.050 | 0 |
| PROST2015924 | 16.951 | 0 | 0 | 0 | 0 | 0 | 0 |
| PROST2017128 | 0 | 0 | 0 | 0 | 0 | 9.026 | 0 |
| PROST2017203 | 1.083 | 0 | 0 | 0 | 0 | 1.455 | 0 |
| PROST2017612 | 0 | 0 | 0 | 0 | 0 | 24.004 | 0 |
| PROST2019164 | 3.075 | 0 | 0 | 0 | 0 | 12.391 | 0 |
| PROST2019253 | 0 | 0 | 0 | 0 | 0 | 1.882 | 0 |
| PUAEN1000057 | 0 | 0 | 0 | 0 | 0 | 2.300 | 0 |
| PUAEN1000065 | 0 | 0 | 0 | 0 | 0 | 0.750 | 0 |
| PUAEN1000147 | 7.775 | 0 | 0 | 0 | 0 | 20.890 | 0 |
| PUAEN1000161 | 0 | 0 | 0 | 0 | 0 | 6.461 | 0 |
| PUAEN1000164 | 3.319 | 0 | 0 | 0 | 0 | 1.338 | 0 |
| PUAEN2000152 | 0 | 0 | 0 | 0 | 0 | 6.648 | 0 |
| PUAEN2000312 | 7.382 | 0 | 0 | 0 | 0 | 0 | 0 |
| PUAEN2000374 | 1.334 | 0 | 0 | 0 | 0 | 1.076 | 0 |
| PUAEN2000535 | 2.012 | 0 | 0 | 0 | 0 | 0.901 | 0 |
| PUAEN2001586 | 0 | 0 | 0 | 0 | 62.197 | 6.397 | 0 |
| PUAEN2004400 | 0 | 26.668 | 0 | 0 | 0 | 0 | 0 |
| PUAEN2005930 | 0 | 0 | 0 | 0 | 0 | 10.284 | 0 |
| PUAEN2006328 | 0 | 0 | 0 | 0 | 0 | 0 | 79.193 |
| PUAEN2007044 | 1.195 | 0 | 0 | 0 | 0 | 14.450 | 0 |
| PUAEN2008939 | 0 | 0 | 0 | 0 | 0 | 3.844 | 0 |
| PUAEN2008985 | 3.700 | 0 | 0 | 0 | 0 | 4.970 | 0 |
| PUAEN2009174 | 2.363 | 0 | 0 | 0 | 0 | 0 | 0 |
| PUAEN2010346 | 8.323 | 0 | 0 | 0 | 0 | 0 | 0 |
| RECTM2002172 | 0 | 14.539 | 0 | 0 | 0 | 0 | 0 |
| SALGL1000047 | 0.247 | 0 | 0 | 0 | 0 | 0 | 0 |
| SALGL1000157 | 0.759 | 0 | 5.364 | 0 | 0 | 0.146 | 0 |
| SALGL1000171 | 0.514 | 0 | 0 | 0 | 0 | 0 | 0 |
| SKMUS1000014 | 0 | 27.230 | 0 | 0 | 0 | 0 | 0 |
| SKMUS1000022 | 0 | 0 | 0 | 0 | 0 | 3.730 | 0 |
| SKMUS1000084 | 5.137 | 0 | 0 | 0 | 0 | 1.725 | 0 |
| SKMUS1000110 | 0 | 27.270 | 0 | 0 | 0 | 0 | 0 |
| SKMUS1000177 | 1.969 | 0 | 0 | 0 | 0 | 26.451 | 0 |
| SKMUS1000186 | 1.339 | 4.457 | 0 | 0 | 0 | 0.450 | 0 |
| SKMUS2000343 | 0 | 0 | 0 | 0 | 0 | 8.630 | 0 |
| SKMUS2000484 | 1.464 | 0 | 0 | 0 | 0 | 0 | 0 |
| SKMUS2000679 | 0 | 0 | 18.423 | 0 | 0 | 0 | 0 |
| SKMUS2000757 | 0 | 18.292 | 0 | 0 | 0 | 0 | 0 |
| SKMUS2000931 | 1.361 | 6.043 | 0 | 0 | 0 | 1.829 | 0 |
| SKMUS2000933 | 0 | 0 | 0 | 0 | 0 | 6.950 | 0 |
| SKMUS2000954 | 0 | 0 | 0 | 0 | 37.020 | 0 | 0 |
| SKMUS2001454 | 0.281 | 1.249 | 0 | 0 | 0 | 0.378 | 0 |
| SKMUS2001528 | 0 | 0 | 0 | 0 | 0 | 0 | 26.957 |
| SKMUS2001634 | 0 | 0 | 0 | 0 | 0 | 1.466 | 0 |
| SKMUS2004908 | 0 | 0 | 0 | 0 | 0 | 3.679 | 0 |
| SKNMC1000137 | 0.884 | 0 | 0 | 0 | 0 | 0 | 0 |
| SKNMC1000168 | 0.698 | 0 | 0 | 0 | 0 | 0.938 | 0 |
| SKNMC1000229 | 4.384 | 0 | 0 | 0 | 0 | 0 | 0 |
| SKNMC1000251 | 5.849 | 0 | 0 | 0 | 0 | 0 | 0 |
| SKNMC1000264 | 4.374 | 0 | 0 | 0 | 0 | 0 | 0 |
| SKNMC2000065 | 1.800 | 0 | 0 | 0 | 0 | 0 | 0 |
| SKNMC2000097 | 1.115 | 0 | 0 | 0 | 0 | 0 | 0 |
| SKNMC2000224 | 5.223 | 0 | 0 | 0 | 0 | 0 | 0 |
| SKNMC2000356 | 14.745 | 0 | 0 | 0 | 0 | 0 | 0 |
| SKNMC2000649 | 0 | 0 | 0 | 0 | 0 | 4.088 | 19.593 |
| SKNMC2000698 | 5.167 | 0 | 0 | 0 | 0 | 0 | 0 |
| SKNMC2000877 | 1.976 | 4.386 | 0 | 0 | 0 | 1.327 | 0 |
| SKNMC2006173 | 0 | 0 | 0 | 0 | 0 | 8.620 | 0 |
| SKNSH1000002 | 0 | 0 | 0 | 0 | 31.308 | 0 | 0 |
| SKNSH1000101 | 0.536 | 0 | 0 | 0 | 0 | 0 | 0 |
| SKNSH2000101 | 0 | 0 | 0 | 0 | 0 | 14.763 | 0 |
| SKNSH2000250 | 0 | 5.215 | 0 | 0 | 0 | 0 | 0 |
| SKNSH2000347 | 0 | 0 | 0 | 0 | 0 | 2.513 | 0 |
| SKNSH2000819 | 2.075 | 0 | 0 | 0 | 0 | 2.788 | 0 |
| SKNSH2001222 | 0 | 0 | 0 | 0 | 0 | 7.314 | 0 |
| SKNSH2002325 | 0 | 0 | 0 | 0 | 0 | 4.276 | 0 |
| SKNSH2002768 | 5.923 | 0 | 0 | 0 | 0 | 0 | 0 |
| SKNSH2003528 | 0 | 0 | 0 | 0 | 0 | 3.400 | 0 |
| SKNSH2006234 | 0 | 0 | 0 | 0 | 0 | 3.921 | 0 |
| SMINT1000016 | 0 | 9.081 | 33.733 | 0 | 0 | 0 | 0 |
| SMINT1000042 | 0 | 0 | 0 | 0 | 0 | 6.002 | 0 |
| SMINT1000117 | 1.097 | 0 | 0 | 0 | 0 | 2.947 | 0 |
| SMINT1000192 | 2.483 | 0 | 0 | 0 | 0 | 0 | 0 |
| SMINT2000007 | 10.031 | 0 | 0 | 0 | 0 | 0 | 0 |
| SMINT2000159 | 0 | 20.230 | 0 | 0 | 0 | 6.122 | 0 |
| SMINT2000232 | 11.871 | 0 | 0 | 0 | 0 | 0 | 0 |
| SMINT2000239 | 0 | 0 | 0 | 0 | 0 | 9.395 | 0 |
| SMINT2000277 | 1.512 | 0 | 0 | 0 | 0 | 2.031 | 0 |
| SMINT2000396 | 2.041 | 4.531 | 0 | 0 | 0 | 8.227 | 0 |
| SMINT2000441 | 0.753 | 3.342 | 0 | 0 | 0 | 1.011 | 0 |
| SMINT2000454 | 0 | 0 | 0 | 0 | 0 | 20.021 | 0 |
| SMINT2000468 | 0 | 0 | 0 | 0 | 0 | 2.563 | 0 |
| SMINT2000545 | 21.744 | 0 | 16.298 | 0 | 0 | 7.966 | 0 |
| SMINT2000602 | 0.458 | 0 | 0 | 0 | 0 | 0 | 0 |
| SMINT2000629 | 4.479 | 0 | 0 | 0 | 0 | 3.008 | 0 |
| SMINT2000659 | 0 | 0 | 0 | 0 | 0 | 0.311 | 0 |
| SMINT2000694 | 0.991 | 0 | 0 | 0 | 0 | 0 | 0 |
| SMINT2000747 | 13.665 | 0 | 0 | 0 | 0 | 0 | 0 |
| SMINT2000933 | 0 | 0 | 7.007 | 0 | 0 | 0 | 21.887 |
| SMINT2001818 | 5.472 | 0 | 0 | 0 | 0 | 1.050 | 0 |
| SMINT2002159 | 0 | 0 | 0 | 0 | 0 | 15.360 | 0 |
| SMINT2002224 | 10.556 | 0 | 0 | 0 | 0 | 0 | 0 |
| SMINT2002620 | 1.889 | 0 | 0 | 0 | 0 | 2.538 | 48.656 |
| SMINT2002689 | 5.881 | 0 | 0 | 0 | 0 | 2.633 | 0 |
| SMINT2002882 | 0 | 0 | 0 | 60.089 | 0 | 0 | 0 |
| SMINT2002976 | 4.349 | 0 | 0 | 0 | 0 | 0 | 0 |
| SMINT2003003 | 16.439 | 0 | 0 | 0 | 0 | 0 | 0 |
| SMINT2003074 | 2.286 | 0 | 0 | 0 | 0 | 1.535 | 0 |
| SMINT2003169 | 0 | 1.741 | 0 | 0 | 0 | 0 | 0 |
| SMINT2003340 | 7.350 | 0 | 0 | 0 | 0 | 0 | 0 |
| SMINT2003423 | 9.059 | 0 | 0 | 0 | 0 | 12.169 | 0 |
| SMINT2003505 | 2.626 | 0 | 0 | 0 | 0 | 0 | 0 |
| SMINT2003905 | 0 | 2.866 | 0 | 0 | 0 | 0 | 0 |
| SMINT2004023 | 1.272 | 0 | 0 | 0 | 0 | 0 | 0 |
| SMINT2004037 | 32.093 | 0 | 0 | 0 | 0 | 0 | 0 |
| SMINT2004144 | 0 | 0 | 12.482 | 0 | 0 | 0 | 0 |
| SMINT2004280 | 10.624 | 0 | 0 | 0 | 0 | 0 | 0 |
| SMINT2004589 | 3.342 | 0 | 0 | 0 | 0 | 0 | 0 |
| SMINT2005387 | 19.114 | 0 | 0 | 0 | 0 | 0 | 0 |
| SMINT2005956 | 0 | 19.486 | 0 | 0 | 0 | 2.948 | 0 |
| SMINT2006801 | 13.430 | 0 | 0 | 0 | 0 | 0 | 0 |
| SMINT2007062 | 19.114 | 0 | 0 | 0 | 0 | 0 | 0 |
| SMINT2007187 | 16.793 | 0 | 0 | 0 | 0 | 0 | 0 |
| SMINT2007792 | 6.970 | 0 | 0 | 0 | 0 | 18.725 | 0 |
| SMINT2008921 | 11.102 | 4.107 | 0 | 0 | 0 | 0 | 0 |
| SMINT2008960 | 0.570 | 0 | 4.704 | 0 | 0 | 0 | 0 |
| SMINT2009292 | 5.915 | 0 | 0 | 0 | 0 | 3.973 | 0 |
| SMINT2010084 | 0 | 0 | 0 | 0 | 0 | 15.395 | 0 |
| SMINT2010369 | 19.114 | 0 | 0 | 0 | 0 | 0 | 0 |
| SMINT2010901 | 11.714 | 0 | 0 | 0 | 0 | 0 | 0 |
| SMINT2012179 | 15.491 | 0 | 0 | 0 | 0 | 0 | 0 |
| SMINT2012291 | 3.845 | 0 | 0 | 0 | 0 | 0 | 0 |
| SMINT2013031 | 7.764 | 0 | 0 | 0 | 0 | 0 | 0 |
| SMINT2013032 | 19.946 | 0 | 0 | 0 | 0 | 0 | 0 |
| SMINT2015294 | 0 | 0 | 54.735 | 0 | 0 | 0 | 0 |
| SMINT2017964 | 0 | 0 | 0 | 0 | 0 | 48.865 | 0 |
| SPLEN1000041 | 32.401 | 0 | 0 | 0 | 0 | 0 | 0 |
| SPLEN1000056 | 32.401 | 0 | 0 | 0 | 0 | 0 | 0 |
| SPLEN1000106 | 3.882 | 0 | 0 | 0 | 0 | 20.857 | 0 |
| SPLEN1000116 | 1.238 | 0 | 0 | 0 | 0 | 0.554 | 0 |
| SPLEN1000134 | 0 | 0 | 0 | 0 | 0 | 39.169 | 0 |
| SPLEN1000166 | 0.943 | 0 | 0 | 0 | 0 | 1.266 | 0 |
| SPLEN2000047 | 13.760 | 0 | 0 | 0 | 0 | 0 | 0 |
| SPLEN2000072 | 5.008 | 0 | 0 | 0 | 0 | 0 | 0 |
| SPLEN2000098 | 0 | 0 | 0 | 0 | 0 | 13.492 | 0 |
| SPLEN2000143 | 19.333 | 0 | 0 | 0 | 0 | 0 | 0 |
| SPLEN2000243 | 36.254 | 0 | 0 | 0 | 0 | 0 | 0 |
| SPLEN2000255 | 8.964 | 0 | 0 | 0 | 0 | 0 | 0 |
| SPLEN2000267 | 14.604 | 0 | 0 | 0 | 0 | 6.539 | 0 |
| SPLEN2000505 | 2.460 | 1.560 | 0 | 0 | 0 | 0 | 0 |
| SPLEN2000516 | 9.967 | 0 | 0 | 0 | 0 | 0 | 0 |
| SPLEN2000874 | 0 | 0 | 0 | 0 | 0 | 0.546 | 0 |
| SPLEN2000882 | 4.394 | 0 | 0 | 0 | 0 | 3.935 | 0 |
| SPLEN2001141 | 11.811 | 0 | 0 | 0 | 0 | 0 | 0 |
| SPLEN2001157 | 9.174 | 0 | 0 | 0 | 0 | 0 | 0 |
| SPLEN2001278 | 32.401 | 0 | 0 | 0 | 0 | 0 | 0 |
| SPLEN2001476 | 32.401 | 0 | 0 | 0 | 0 | 0 | 0 |
| SPLEN2001710 | 3.233 | 0 | 0 | 0 | 0 | 0 | 83.275 |
| SPLEN2002223 | 0 | 0 | 0 | 0 | 0 | 39.169 | 0 |
| SPLEN2002335 | 2.533 | 3.748 | 0 | 0 | 0 | 3.403 | 0 |
| SPLEN2002451 | 32.401 | 0 | 0 | 0 | 0 | 0 | 0 |
| SPLEN2002463 | 6.314 | 0 | 0 | 0 | 0 | 8.482 | 0 |
| SPLEN2002493 | 0 | 6.407 | 0 | 0 | 0 | 5.816 | 0 |
| SPLEN2002744 | 1.860 | 0 | 0 | 0 | 0 | 0 | 0 |
| SPLEN2002931 | 0 | 0 | 0 | 0 | 88.828 | 0 | 0 |
| SPLEN2003297 | 22.140 | 0 | 0 | 0 | 0 | 0 | 0 |
| SPLEN2004181 | 6.564 | 0 | 0 | 0 | 0 | 0 | 0 |
| SPLEN2004220 | 2.398 | 0 | 0 | 0 | 0 | 3.222 | 0 |
| SPLEN2004555 | 0.888 | 0 | 0 | 0 | 23.184 | 4.769 | 0 |
| SPLEN2004611 | 32.401 | 0 | 0 | 0 | 0 | 0 | 0 |
| SPLEN2004662 | 0 | 0 | 0 | 0 | 0 | 21.098 | 0 |
| SPLEN2004773 | 0.593 | 5.269 | 4.893 | 0 | 0 | 3.986 | 0 |
| SPLEN2004984 | 2.190 | 0 | 0 | 0 | 0 | 0 | 0 |
| SPLEN2005342 | 17.794 | 0 | 0 | 0 | 0 | 0 | 0 |
| SPLEN2005416 | 0 | 0 | 0 | 0 | 0 | 39.169 | 0 |
| SPLEN2005429 | 6.110 | 0 | 0 | 0 | 0 | 8.208 | 0 |
| SPLEN2005560 | 0.878 | 0 | 0 | 0 | 0 | 1.179 | 0 |
| SPLEN2005783 | 5.436 | 24.131 | 0 | 0 | 0 | 0 | 0 |
| SPLEN2005790 | 32.401 | 0 | 0 | 0 | 0 | 0 | 0 |
| SPLEN2005806 | 0.510 | 0 | 0 | 0 | 0 | 0 | 0 |
| SPLEN2005818 | 37.178 | 0 | 0 | 0 | 0 | 0 | 0 |
| SPLEN2006122 | 0 | 0 | 0 | 0 | 0 | 0.943 | 0 |
| SPLEN2006133 | 2.373 | 0 | 0 | 0 | 0 | 0 | 0 |
| SPLEN2006143 | 5.597 | 0 | 0 | 0 | 0 | 2.506 | 0 |
| SPLEN2006232 | 4.784 | 0 | 0 | 0 | 0 | 3.213 | 0 |
| SPLEN2006305 | 13.695 | 0 | 0 | 0 | 0 | 4.953 | 0 |
| SPLEN2006374 | 3.802 | 0 | 0 | 0 | 0 | 3.405 | 0 |
| SPLEN2007182 | 0 | 0 | 0 | 0 | 0 | 14.317 | 0 |
| SPLEN2007326 | 32.401 | 0 | 0 | 0 | 0 | 0 | 0 |
| SPLEN2007388 | 22.140 | 0 | 0 | 0 | 0 | 0 | 0 |
| SPLEN2007498 | 4.364 | 0 | 0 | 0 | 0 | 2.931 | 0 |
| SPLEN2007739 | 10.126 | 0 | 0 | 0 | 0 | 13.603 | 0 |
| SPLEN2007794 | 8.264 | 0 | 0 | 0 | 0 | 0 | 0 |
| SPLEN2007879 | 24.044 | 0 | 0 | 0 | 0 | 0 | 0 |
| SPLEN2008460 | 5.462 | 0 | 0 | 0 | 0 | 1.467 | 0 |
| SPLEN2009081 | 5.397 | 0 | 0 | 0 | 0 | 0 | 0 |
| SPLEN2009163 | 0 | 68.029 | 0 | 0 | 0 | 0 | 0 |
| SPLEN2009512 | 13.810 | 0 | 0 | 0 | 0 | 0 | 0 |
| SPLEN2009513 | 0 | 0 | 42.623 | 0 | 0 | 0 | 0 |
| SPLEN2009970 | 8.505 | 0 | 0 | 0 | 0 | 0 | 0 |
| SPLEN2010015 | 12.963 | 0 | 0 | 0 | 0 | 0 | 0 |
| SPLEN2010195 | 2.588 | 0 | 0 | 0 | 0 | 3.911 | 0 |
| SPLEN2010625 | 32.401 | 0 | 0 | 0 | 0 | 0 | 0 |
| SPLEN2010846 | 19.155 | 0 | 0 | 0 | 0 | 0 | 0 |
| SPLEN2010912 | 10.327 | 0 | 0 | 0 | 0 | 0 | 0 |
| SPLEN2011021 | 13.665 | 0 | 0 | 0 | 0 | 0 | 0 |
| SPLEN2011737 | 8.690 | 0 | 0 | 0 | 0 | 5.837 | 0 |
| SPLEN2011758 | 0 | 0 | 0 | 0 | 0 | 5.883 | 0 |
| SPLEN2011766 | 13.625 | 0 | 0 | 0 | 0 | 0 | 0 |
| SPLEN2012523 | 0 | 0 | 0 | 0 | 0 | 0 | 22.930 |
| SPLEN2012571 | 2.651 | 0 | 0 | 0 | 0 | 0 | 0 |
| SPLEN2012624 | 21.081 | 0 | 0 | 0 | 0 | 0 | 0 |
| SPLEN2012800 | 0.570 | 0 | 0 | 0 | 0 | 0 | 0 |
| SPLEN2012846 | 22.575 | 0 | 0 | 0 | 0 | 30.326 | 0 |
| SPLEN2012889 | 32.401 | 0 | 0 | 0 | 0 | 0 | 0 |
| SPLEN2013108 | 0 | 0 | 0 | 0 | 0 | 39.169 | 0 |
| SPLEN2013670 | 19.554 | 43.403 | 0 | 0 | 0 | 0 | 0 |
| SPLEN2013753 | 32.401 | 0 | 0 | 0 | 0 | 0 | 0 |
| SPLEN2013923 | 22.575 | 0 | 0 | 0 | 0 | 30.326 | 0 |
| SPLEN2014041 | 32.401 | 0 | 0 | 0 | 0 | 0 | 0 |
| SPLEN2014080 | 3.951 | 0 | 0 | 0 | 0 | 0 | 0 |
| SPLEN2014452 | 0 | 68.029 | 0 | 0 | 0 | 0 | 0 |
| SPLEN2014739 | 24.216 | 0 | 0 | 0 | 0 | 0 | 0 |
| SPLEN2014911 | 5.278 | 0 | 0 | 0 | 0 | 1.182 | 0 |
| SPLEN2015121 | 32.401 | 0 | 0 | 0 | 0 | 0 | 0 |
| SPLEN2015310 | 0.478 | 0 | 0 | 0 | 0 | 0 | 0 |
| SPLEN2015788 | 12.109 | 0 | 0 | 0 | 0 | 0 | 0 |
| SPLEN2016098 | 0 | 68.029 | 0 | 0 | 0 | 0 | 0 |
| SPLEN2016139 | 0 | 0 | 0 | 0 | 0 | 1.435 | 0 |
| SPLEN2016268 | 1.801 | 0 | 0 | 8.941 | 0 | 0 | 0 |
| SPLEN2016421 | 0 | 0 | 0 | 0 | 0 | 4.641 | 0 |
| SPLEN2016678 | 65.722 | 0 | 0 | 0 | 0 | 0 | 0 |
| SPLEN2016743 | 4.781 | 0 | 0 | 0 | 0 | 0 | 0 |
| SPLEN2016781 | 32.401 | 0 | 0 | 0 | 0 | 0 | 0 |
| SPLEN2017043 | 8.679 | 0 | 0 | 0 | 0 | 0 | 0 |
| SPLEN2017318 | 16.924 | 0 | 0 | 0 | 0 | 11.367 | 0 |
| SPLEN2017351 | 5.016 | 0 | 0 | 0 | 0 | 10.107 | 0 |
| SPLEN2018157 | 0 | 0 | 0 | 0 | 0 | 5.404 | 0 |
| SPLEN2018299 | 0 | 0 | 0 | 0 | 0 | 23.049 | 0 |
| SPLEN2018683 | 32.401 | 0 | 0 | 0 | 0 | 0 | 0 |
| SPLEN2019077 | 4.424 | 0 | 0 | 0 | 0 | 0 | 0 |
| SPLEN2019169 | 18.883 | 0 | 0 | 0 | 0 | 6.341 | 0 |
| SPLEN2019405 | 7.165 | 0 | 0 | 0 | 0 | 0 | 0 |
| SPLEN2019446 | 8.677 | 0 | 0 | 0 | 0 | 0 | 0 |
| SPLEN2019505 | 4.088 | 0 | 0 | 0 | 0 | 0 | 0 |
| SPLEN2019956 | 25.397 | 0 | 0 | 0 | 0 | 0 | 0 |
| SPLEN2021194 | 2.813 | 0 | 0 | 0 | 0 | 0 | 0 |
| SPLEN2022522 | 2.364 | 0 | 0 | 0 | 0 | 0 | 0 |
| SPLEN2023118 | 0 | 41.203 | 0 | 0 | 0 | 0 | 0 |
| SPLEN2023733 | 16.788 | 0 | 0 | 0 | 0 | 0 | 0 |
| SPLEN2024232 | 0 | 0 | 45.601 | 0 | 0 | 0 | 0 |
| SPLEN2024489 | 0 | 0 | 0 | 0 | 0 | 12.238 | 0 |
| SPLEN2024922 | 0 | 0 | 0 | 0 | 0 | 28.869 | 0 |
| SPLEN2024990 | 19.333 | 0 | 0 | 0 | 0 | 0 | 0 |
| SPLEN2027113 | 0 | 0 | 0 | 0 | 0 | 5.499 | 0 |
| SPLEN2028466 | 0.690 | 1.531 | 0 | 0 | 0 | 0.463 | 0 |
| SPLEN2028914 | 0 | 0 | 0 | 0 | 0 | 3.128 | 0 |
| SPLEN2029295 | 0 | 0 | 0 | 0 | 0 | 39.169 | 0 |
| SPLEN2029683 | 0 | 0 | 0 | 0 | 0 | 39.169 | 0 |
| SPLEN2029912 | 1.362 | 0 | 0 | 0 | 0 | 0 | 0 |
| SPLEN2031547 | 10.503 | 0 | 0 | 0 | 0 | 3.527 | 0 |
| SPLEN2031674 | 36.926 | 0 | 0 | 0 | 0 | 0 | 0 |
| SPLEN2032157 | 4.765 | 0 | 0 | 0 | 0 | 0 | 0 |
| SPLEN2033098 | 16.597 | 0 | 34.211 | 0 | 0 | 5.574 | 0 |
| SPLEN2034448 | 17.293 | 0 | 0 | 0 | 0 | 5.808 | 0 |
| SPLEN2034781 | 23.334 | 0 | 0 | 0 | 0 | 0 | 0 |
| SPLEN2036076 | 21.918 | 0 | 0 | 0 | 0 | 1.840 | 35.281 |
| SPLEN2036501 | 32.401 | 0 | 0 | 0 | 0 | 0 | 0 |
| SPLEN2037722 | 1.374 | 0 | 0 | 0 | 0 | 1.846 | 0 |
| SPLEN2038834 | 0 | 34.296 | 0 | 0 | 0 | 0 | 0 |
| SPLEN2039379 | 2.415 | 0 | 0 | 0 | 0 | 0 | 0 |
| SPLEN2039672 | 32.401 | 0 | 0 | 0 | 0 | 0 | 0 |
| SPLEN2041306 | 44.955 | 0 | 0 | 0 | 0 | 0 | 0 |
| SPLEN2042303 | 21.853 | 0 | 0 | 0 | 0 | 0 | 0 |
| SPLEN2042535 | 5.937 | 0 | 0 | 0 | 0 | 0 | 0 |
| STOMA1000047 | 13.211 | 0 | 0 | 0 | 0 | 0 | 0 |
| STOMA1000189 | 0 | 0 | 0 | 0 | 0 | 3.503 | 0 |
| STOMA2000121 | 0 | 4.511 | 0 | 0 | 0 | 0 | 0 |
| STOMA2000257 | 0 | 7.307 | 0 | 0 | 0 | 2.211 | 0 |
| STOMA2000395 | 8.900 | 0 | 0 | 0 | 0 | 0 | 0 |
| STOMA2002052 | 4.638 | 0 | 0 | 0 | 0 | 12.460 | 0 |
| STOMA2002166 | 0.488 | 0 | 0 | 12.107 | 0 | 0 | 0 |
| STOMA2002828 | 0 | 0 | 0 | 0 | 0 | 6.139 | 0 |
| STOMA2003158 | 5.104 | 0 | 0 | 0 | 0 | 0 | 0 |
| STOMA2003716 | 9.520 | 0 | 0 | 0 | 0 | 12.788 | 0 |
| STOMA2003781 | 0 | 0 | 0 | 0 | 0 | 14.134 | 0 |
| STOMA2009289 | 0 | 0 | 0 | 0 | 0 | 6.426 | 0 |
| SYNOV1000124 | 0.928 | 0 | 0 | 0 | 12.116 | 1.246 | 0 |
| SYNOV1000164 | 6.413 | 0 | 0 | 0 | 0 | 0 | 0 |
| SYNOV1000374 | 0 | 0 | 0 | 0 | 0 | 22.227 | 0 |
| SYNOV2001033 | 2.000 | 0 | 0 | 0 | 0 | 1.343 | 0 |
| SYNOV2001239 | 7.587 | 0 | 0 | 6.493 | 13.668 | 0 | 6.738 |
| SYNOV2001262 | 6.959 | 0 | 0 | 0 | 0 | 4.674 | 0 |
| SYNOV2001374 | 1.527 | 0 | 0 | 0 | 0 | 0 | 0 |
| SYNOV2001581 | 0 | 9.748 | 0 | 0 | 0 | 0 | 0 |
| SYNOV2001648 | 0 | 0 | 7.450 | 44.851 | 0 | 0 | 0 |
| SYNOV2005541 | 0 | 0 | 0 | 0 | 0 | 16.036 | 0 |
| SYNOV2007758 | 16.315 | 0 | 0 | 0 | 0 | 21.917 | 0 |
| SYNOV2009172 | 0.434 | 0 | 0 | 0 | 0 | 0 | 0 |
| SYNOV2017055 | 14.078 | 0 | 0 | 0 | 0 | 0 | 0 |
| SYNOV4001153 | 5.802 | 0 | 0 | 0 | 0 | 2.598 | 0 |
| SYNOV4001326 | 0 | 0 | 0 | 0 | 0 | 34.182 | 0 |
| SYNOV4004823 | 0 | 0 | 0 | 0 | 0 | 50.949 | 0 |
| SYNOV4005989 | 34.423 | 0 | 0 | 0 | 0 | 0 | 0 |
| SYNOV4006327 | 12.888 | 0 | 0 | 0 | 0 | 0 | 0 |
| SYNOV4006542 | 0 | 0 | 0 | 0 | 0 | 4.153 | 0 |
| SYNOV4007012 | 27.882 | 0 | 0 | 0 | 0 | 0 | 0 |
| SYNOV4007553 | 27.882 | 0 | 0 | 0 | 0 | 0 | 0 |
| SYNOV4007671 | 0 | 5.427 | 0 | 0 | 0 | 0 | 0 |
| SYNOV4008245 | 15.407 | 0 | 0 | 0 | 0 | 10.349 | 0 |
| T1ESE2000116 | 3.618 | 0 | 0 | 0 | 0 | 0 | 0 |
| T1ESE2000609 | 1.895 | 0 | 0 | 0 | 0 | 0 | 0 |
| T1ESE2000904 | 0 | 0 | 0 | 0 | 0 | 2.586 | 0 |
| TBAES2001296 | 9.394 | 0 | 0 | 0 | 0 | 0 | 0 |
| TBAES2001553 | 0 | 10.888 | 0 | 0 | 0 | 0 | 0 |
| TCOLN1000180 | 2.041 | 0 | 0 | 0 | 0 | 0 | 0 |
| TESOP2001953 | 10.863 | 0 | 0 | 0 | 0 | 0 | 0 |
| TESOP2002451 | 0 | 0 | 16.330 | 0 | 0 | 0 | 0 |
| TESOP2007384 | 0 | 0 | 0 | 0 | 0 | 4.497 | 0 |
| TESTI1000023 | 2.098 | 3.104 | 0 | 0 | 0 | 3.758 | 0 |
| TESTI1000051 | 0 | 0 | 0 | 0 | 0 | 17.501 | 0 |
| TESTI1000096 | 14.587 | 0 | 0 | 0 | 0 | 0 | 0 |
| TESTI1000138 | 24.890 | 0 | 0 | 0 | 0 | 0 | 0 |
| TESTI1000142 | 0 | 0 | 0 | 0 | 0 | 22.673 | 0 |
| TESTI1000168 | 0 | 0 | 0 | 0 | 0 | 14.041 | 0 |
| TESTI2000006 | 8.245 | 0 | 0 | 0 | 0 | 0 | 0 |
| TESTI2000177 | 0 | 0 | 0 | 0 | 0 | 27.311 | 0 |
| TESTI2000179 | 30.546 | 0 | 0 | 0 | 0 | 0 | 0 |
| TESTI2000184 | 3.770 | 0 | 0 | 0 | 0 | 11.396 | 0 |
| TESTI2000272 | 0 | 0 | 0 | 0 | 0 | 10.846 | 0 |
| TESTI2000431 | 0 | 0 | 0 | 0 | 0 | 36.354 | 0 |
| TESTI2000520 | 0 | 0 | 0 | 0 | 0 | 4.214 | 0 |
| TESTI2000598 | 4.189 | 0 | 0 | 0 | 0 | 0 | 0 |
| TESTI2000627 | 0 | 0 | 0 | 0 | 0 | 23.601 | 0 |
| TESTI2000784 | 7.580 | 0 | 0 | 0 | 0 | 5.091 | 0 |
| TESTI2000970 | 0 | 17.994 | 0 | 0 | 0 | 0 | 0 |
| TESTI2001099 | 4.495 | 1.814 | 0 | 0 | 0 | 4.391 | 0 |
| TESTI2001120 | 0.806 | 0 | 0 | 0 | 0 | 0 | 0 |
| TESTI2001153 | 0 | 0 | 0 | 0 | 0 | 0.446 | 0 |
| TESTI2001269 | 0.455 | 0 | 0 | 0 | 0 | 2.446 | 0 |
| TESTI2001511 | 0 | 0 | 0 | 0 | 0 | 1.960 | 0 |
| TESTI2001661 | 45.958 | 0 | 0 | 0 | 0 | 0 | 0 |
| TESTI2001862 | 4.410 | 0 | 0 | 0 | 0 | 2.962 | 0 |
| TESTI2001879 | 0 | 0 | 0 | 0 | 0 | 63.148 | 0 |
| TESTI2002105 | 0 | 0 | 0 | 0 | 0 | 14.629 | 0 |
| TESTI2002223 | 0 | 0 | 0 | 0 | 0 | 30.804 | 0 |
| TESTI2002256 | 0 | 0 | 0 | 0 | 0 | 38.856 | 0 |
| TESTI2002365 | 5.463 | 0 | 15.015 | 0 | 23.785 | 1.223 | 0 |
| TESTI2002369 | 0 | 0 | 0 | 0 | 0 | 16.832 | 0 |
| TESTI2002707 | 0 | 0 | 0 | 0 | 0 | 1.856 | 0 |
| TESTI2002866 | 17.744 | 0 | 0 | 0 | 0 | 11.918 | 0 |
| TESTI2002877 | 31.111 | 0 | 0 | 0 | 0 | 0 | 0 |
| TESTI2002965 | 68.011 | 0 | 0 | 0 | 0 | 0 | 0 |
| TESTI2003109 | 0 | 0 | 0 | 0 | 0 | 25.524 | 0 |
| TESTI2003280 | 0 | 15.184 | 0 | 0 | 0 | 0 | 0 |
| TESTI2003413 | 2.171 | 0 | 0 | 0 | 0 | 0 | 0 |
| TESTI2003454 | 4.428 | 0 | 0 | 0 | 0 | 5.948 | 0 |
| TESTI2003533 | 0.723 | 0 | 0 | 0 | 0 | 0 | 0 |
| TESTI2003625 | 0 | 0 | 0 | 0 | 0 | 25.720 | 0 |
| TESTI2004031 | 0.334 | 0 | 0 | 0 | 8.732 | 0 | 0 |
| TESTI2004080 | 0 | 0 | 33.552 | 0 | 0 | 0 | 0 |
| TESTI2004295 | 0 | 0 | 0 | 0 | 0 | 9.172 | 0 |
| TESTI2004432 | 0 | 0 | 0 | 0 | 0 | 63.148 | 0 |
| TESTI2004737 | 2.954 | 0 | 0 | 0 | 0 | 0 | 0 |
| TESTI2005408 | 1.477 | 6.558 | 0 | 0 | 0 | 0 | 0 |
| TESTI2005564 | 0 | 0 | 0 | 0 | 0 | 8.204 | 0 |
| TESTI2005692 | 0 | 0 | 0 | 0 | 0 | 63.148 | 0 |
| TESTI2005731 | 0 | 0 | 0 | 0 | 0 | 37.009 | 0 |
| TESTI2005743 | 1.832 | 0 | 0 | 0 | 0 | 0 | 47.175 |
| TESTI2005759 | 0 | 0 | 0 | 0 | 0 | 1.048 | 0 |
| TESTI2005784 | 0 | 0 | 0 | 0 | 0 | 9.995 | 0 |
| TESTI2005788 | 3.856 | 0 | 31.796 | 0 | 0 | 0 | 0 |
| TESTI2005908 | 0 | 0 | 0 | 0 | 0 | 1.764 | 0 |
| TESTI2006120 | 0 | 17.965 | 0 | 0 | 0 | 0 | 0 |
| TESTI2006258 | 8.550 | 0 | 0 | 0 | 0 | 0 | 0 |
| TESTI2006383 | 0 | 0 | 0 | 0 | 0 | 4.540 | 0 |
| TESTI2006453 | 2.807 | 0 | 0 | 0 | 0 | 0 | 0 |
| TESTI2006588 | 2.257 | 0 | 0 | 0 | 0 | 0.606 | 0 |
| TESTI2006615 | 0 | 0 | 0 | 0 | 0 | 5.170 | 0 |
| TESTI2006720 | 56.056 | 0 | 0 | 0 | 0 | 0 | 0 |
| TESTI2006872 | 4.582 | 0 | 0 | 0 | 0 | 24.621 | 0 |
| TESTI2006877 | 0 | 0 | 0 | 0 | 0 | 14.866 | 0 |
| TESTI2007211 | 8.262 | 12.226 | 0 | 0 | 0 | 0 | 0 |
| TESTI2007407 | 0 | 22.724 | 0 | 0 | 0 | 0 | 0 |
| TESTI2007422 | 25.838 | 0 | 0 | 0 | 0 | 0 | 0 |
| TESTI2007464 | 3.344 | 0 | 0 | 0 | 0 | 0 | 0 |
| TESTI2007864 | 56.056 | 0 | 0 | 0 | 0 | 0 | 0 |
| TESTI2007998 | 1.318 | 0 | 0 | 0 | 0 | 0 | 0 |
| TESTI2008020 | 3.198 | 0 | 0 | 0 | 0 | 4.296 | 0 |
| TESTI2008240 | 0.834 | 0.926 | 0 | 0 | 0 | 1.961 | 0 |
| TESTI2008405 | 0 | 0 | 35.801 | 0 | 0 | 0 | 0 |
| TESTI2009402 | 7.644 | 0 | 0 | 0 | 0 | 20.538 | 0 |
| TESTI2009474 | 5.054 | 0 | 0 | 0 | 0 | 2.263 | 0 |
| TESTI2009502 | 0 | 0 | 0 | 0 | 0 | 0 | 97.046 |
| TESTI2009520 | 2.501 | 0 | 0 | 0 | 0 | 4.200 | 0 |
| TESTI2009577 | 5.895 | 0 | 0 | 0 | 0 | 3.959 | 0 |
| TESTI2009785 | 9.571 | 0 | 0 | 0 | 0 | 3.214 | 0 |
| TESTI2009987 | 0 | 0 | 0 | 0 | 0 | 5.714 | 0 |
| TESTI2010180 | 0 | 0 | 0 | 0 | 0 | 38.856 | 0 |
| TESTI2010513 | 1.966 | 0 | 0 | 0 | 0 | 5.281 | 0 |
| TESTI2012394 | 0 | 0 | 0 | 0 | 0 | 29.991 | 0 |
| TESTI2012778 | 0.616 | 0 | 0 | 0 | 0 | 0 | 0 |
| TESTI2013231 | 0 | 0 | 13.680 | 0 | 0 | 0 | 0 |
| TESTI2013264 | 20.949 | 0 | 0 | 0 | 0 | 0 | 0 |
| TESTI2013295 | 2.221 | 0 | 0 | 0 | 0 | 5.967 | 0 |
| TESTI2014505 | 16.432 | 0 | 0 | 0 | 0 | 0 | 0 |
| TESTI2014838 | 1.105 | 0 | 0 | 0 | 0 | 11.873 | 0 |
| TESTI2015092 | 0 | 0 | 0 | 0 | 0 | 7.422 | 0 |
| TESTI2015213 | 1.284 | 0 | 0 | 0 | 0 | 0 | 11.022 |
| TESTI2015626 | 0.548 | 4.869 | 2.261 | 0 | 7.163 | 1.105 | 0 |
| TESTI2015962 | 0 | 14.684 | 0 | 0 | 0 | 0 | 0 |
| TESTI2016033 | 0 | 0 | 11.978 | 36.053 | 0 | 0 | 37.415 |
| TESTI2017954 | 2.210 | 4.904 | 0 | 0 | 0 | 0.742 | 0 |
| TESTI2018014 | 3.214 | 0 | 0 | 0 | 0 | 4.318 | 0 |
| TESTI2018462 | 0 | 0 | 0 | 0 | 0 | 6.331 | 0 |
| TESTI2018867 | 2.374 | 0 | 0 | 0 | 0 | 0 | 0 |
| TESTI2018997 | 1.737 | 0 | 0 | 0 | 0 | 0 | 0 |
| TESTI2019077 | 56.056 | 0 | 0 | 0 | 0 | 0 | 0 |
| TESTI2019760 | 37.949 | 0 | 0 | 0 | 0 | 0 | 0 |
| TESTI2020510 | 6.032 | 0 | 0 | 0 | 0 | 8.102 | 0 |
| TESTI2020840 | 13.626 | 0 | 0 | 0 | 0 | 0 | 0 |
| TESTI2020956 | 0 | 0 | 0 | 0 | 0 | 0 | 92.415 |
| TESTI2021124 | 0 | 0 | 0 | 0 | 0 | 12.804 | 0 |
| TESTI2021297 | 5.724 | 0 | 0 | 0 | 0 | 0 | 0 |
| TESTI2021425 | 2.871 | 0 | 0 | 0 | 0 | 1.928 | 0 |
| TESTI2022179 | 8.758 | 0 | 0 | 0 | 0 | 0 | 0 |
| TESTI2023414 | 0 | 0 | 0 | 0 | 0 | 50.153 | 0 |
| TESTI2024283 | 0 | 0 | 0 | 0 | 0 | 63.148 | 0 |
| TESTI2024844 | 0 | 4.241 | 0 | 0 | 0 | 0 | 0 |
| TESTI2024864 | 0 | 0 | 0 | 0 | 0 | 30.932 | 0 |
| TESTI2025757 | 14.139 | 0 | 0 | 0 | 0 | 0 | 0 |
| TESTI2027271 | 4.403 | 0 | 0 | 0 | 0 | 0 | 0 |
| TESTI2027909 | 56.056 | 0 | 0 | 0 | 0 | 0 | 0 |
| TESTI2028659 | 25.135 | 0 | 0 | 0 | 0 | 33.765 | 0 |
| TESTI2029227 | 0 | 0 | 0 | 0 | 0 | 13.298 | 0 |
| TESTI2029259 | 2.760 | 0 | 0 | 0 | 0 | 3.707 | 0 |
| TESTI2029785 | 56.056 | 0 | 0 | 0 | 0 | 0 | 0 |
| TESTI2030336 | 56.056 | 0 | 0 | 0 | 0 | 0 | 0 |
| TESTI2030554 | 29.835 | 0 | 0 | 0 | 0 | 0 | 0 |
| TESTI2031007 | 0 | 0 | 0 | 0 | 0 | 36.354 | 0 |
| TESTI2032643 | 10.140 | 0 | 0 | 0 | 0 | 0 | 0 |
| TESTI2033031 | 25.656 | 0 | 0 | 0 | 0 | 0 | 0 |
| TESTI2033441 | 7.320 | 0 | 0 | 0 | 0 | 0 | 0 |
| TESTI2034307 | 56.056 | 0 | 0 | 0 | 0 | 0 | 0 |
| TESTI2034916 | 6.339 | 0 | 0 | 0 | 0 | 8.515 | 0 |
| TESTI2034940 | 1.521 | 1.929 | 0 | 0 | 5.674 | 0.292 | 0 |
| TESTI2036913 | 0 | 0 | 0 | 0 | 0 | 22.732 | 0 |
| TESTI2036969 | 2.996 | 0 | 0 | 0 | 0 | 0 | 0 |
| TESTI2039177 | 0 | 0 | 0 | 0 | 0 | 9.285 | 0 |
| TESTI2039707 | 0 | 84.991 | 0 | 0 | 0 | 0 | 0 |
| TESTI2044276 | 3.954 | 0 | 0 | 0 | 0 | 10.623 | 0 |
| TESTI2046456 | 1.544 | 3.427 | 3.182 | 0 | 0 | 5.185 | 0 |
| TESTI2047605 | 0 | 0 | 0 | 0 | 0 | 16.321 | 0 |
| TESTI2053667 | 0 | 0 | 0 | 0 | 0 | 63.148 | 0 |
| TESTI2053723 | 3.775 | 0 | 0 | 0 | 0 | 0 | 0 |
| TESTI4000014 | 0.596 | 0 | 0 | 0 | 0 | 2.800 | 3.835 |
| TESTI4000349 | 0 | 0 | 0 | 0 | 0 | 29.117 | 0 |
| TESTI4000434 | 0 | 0 | 0 | 0 | 0 | 14.646 | 0 |
| TESTI4001467 | 4.616 | 0 | 0 | 0 | 0 | 0 | 0 |
| TESTI4001561 | 0 | 0 | 0 | 0 | 0 | 45.102 | 0 |
| TESTI4002202 | 0 | 0 | 22.792 | 0 | 0 | 0 | 0 |
| TESTI4002799 | 0.368 | 0 | 0 | 0 | 0 | 1.979 | 0 |
| TESTI4002988 | 0 | 0 | 0 | 0 | 0 | 48.582 | 0 |
| TESTI4003150 | 12.383 | 0 | 0 | 0 | 0 | 0 | 0 |
| TESTI4003179 | 0 | 0 | 0 | 0 | 0 | 46.144 | 0 |
| TESTI4003579 | 0 | 0 | 0 | 0 | 0 | 63.148 | 0 |
| TESTI4003602 | 21.653 | 0 | 0 | 0 | 0 | 0 | 0 |
| TESTI4003703 | 0 | 0 | 0 | 0 | 0 | 4.956 | 0 |
| TESTI4004210 | 0 | 0 | 0 | 0 | 0 | 40.382 | 0 |
| TESTI4005133 | 9.943 | 0 | 0 | 0 | 0 | 13.357 | 0 |
| TESTI4005399 | 6.502 | 0 | 0 | 0 | 0 | 8.734 | 0 |
| TESTI4006137 | 0 | 0 | 0 | 0 | 0 | 28.849 | 0 |
| TESTI4006819 | 0 | 0 | 0 | 0 | 0 | 46.144 | 0 |
| TESTI4007816 | 0 | 0 | 0 | 89.634 | 0 | 0 | 0 |
| TESTI4009286 | 3.627 | 0 | 0 | 0 | 0 | 4.060 | 0 |
| TESTI4009454 | 0 | 0 | 0 | 0 | 0 | 9.004 | 0 |
| TESTI4009457 | 0 | 0 | 0 | 0 | 0 | 29.991 | 0 |
| TESTI4010851 | 3.191 | 0 | 0 | 0 | 0 | 1.837 | 0 |
| TESTI4010979 | 16.384 | 0 | 0 | 0 | 0 | 0 | 0 |
| TESTI4013441 | 0.915 | 0 | 1.078 | 0 | 0 | 0.176 | 0 |
| TESTI4013474 | 8.616 | 0 | 7.104 | 0 | 0 | 0 | 0 |
| TESTI4013735 | 0 | 0 | 0 | 0 | 0 | 63.148 | 0 |
| TESTI4014908 | 0 | 0 | 0 | 0 | 0 | 40.382 | 0 |
| TESTI4016925 | 0 | 0 | 0 | 0 | 0 | 77.412 | 0 |
| TESTI4018152 | 71.841 | 0 | 0 | 0 | 0 | 0 | 0 |
| TESTI4018938 | 0 | 0 | 0 | 0 | 0 | 19.395 | 0 |
| TESTI4024245 | 0 | 0 | 0 | 0 | 0 | 37.759 | 0 |
| TESTI4024740 | 15.000 | 0 | 0 | 0 | 0 | 0 | 0 |
| TESTI4026079 | 0 | 0 | 0 | 0 | 0 | 28.497 | 0 |
| TESTI4029671 | 0 | 0 | 0 | 0 | 0 | 20.833 | 0 |
| TESTI4032913 | 6.759 | 0 | 0 | 0 | 0 | 0 | 0 |
| TESTI4033690 | 0 | 0 | 0 | 0 | 0 | 63.148 | 0 |
| TESTI4037156 | 0.923 | 0 | 0 | 0 | 0 | 0.620 | 0 |
| TESTI4038284 | 0 | 0 | 0 | 0 | 0 | 4.126 | 0 |
| TESTI4043140 | 4.624 | 0 | 0 | 0 | 0 | 3.106 | 0 |
| TESTI4044704 | 4.250 | 0 | 0 | 0 | 0 | 0 | 0 |
| TESTI4046819 | 0 | 73.082 | 0 | 0 | 0 | 0 | 0 |
| THYMU1000002 | 0.494 | 2.191 | 0 | 0 | 0 | 0.663 | 0 |
| THYMU1000010 | 4.496 | 0 | 0 | 0 | 0 | 0 | 0 |
| THYMU1000016 | 1.289 | 0 | 0 | 0 | 0 | 1.299 | 0 |
| THYMU1000017 | 25.065 | 0 | 0 | 0 | 0 | 0 | 0 |
| THYMU1000025 | 100 | 0 | 0 | 0 | 0 | 0 | 0 |
| THYMU1000029 | 44.974 | 0 | 0 | 0 | 0 | 0 | 0 |
| THYMU1000032 | 50.116 | 0 | 0 | 0 | 0 | 0.874 | 0 |
| THYMU1000033 | 100 | 0 | 0 | 0 | 0 | 0 | 0 |
| THYMU1000041 | 11.897 | 0 | 0 | 0 | 0 | 13.318 | 0 |
| THYMU1000060 | 100 | 0 | 0 | 0 | 0 | 0 | 0 |
| THYMU1000074 | 100 | 0 | 0 | 0 | 0 | 0 | 0 |
| THYMU1000083 | 7.501 | 0 | 0 | 0 | 0 | 3.359 | 0 |
| THYMU1000098 | 100 | 0 | 0 | 0 | 0 | 0 | 0 |
| THYMU1000103 | 14.701 | 0 | 0 | 0 | 0 | 1.975 | 0 |
| THYMU1000105 | 12.511 | 55.541 | 0 | 0 | 0 | 0 | 0 |
| THYMU1000109 | 2.625 | 0 | 0 | 0 | 17.143 | 0 | 0 |
| THYMU1000136 | 100 | 0 | 0 | 0 | 0 | 0 | 0 |
| THYMU1000142 | 41.147 | 0 | 0 | 0 | 0 | 0 | 0 |
| THYMU1000144 | 32.353 | 0 | 0 | 0 | 0 | 0 | 0 |
| THYMU1000158 | 100 | 0 | 0 | 0 | 0 | 0 | 0 |
| THYMU1000176 | 100 | 0 | 0 | 0 | 0 | 0 | 0 |
| THYMU1000179 | 42.674 | 0 | 0 | 0 | 0 | 57.326 | 0 |
| THYMU1000191 | 100 | 0 | 0 | 0 | 0 | 0 | 0 |
| THYMU1000198 | 45.282 | 0 | 0 | 0 | 0 | 0 | 0 |
| THYMU1000217 | 100 | 0 | 0 | 0 | 0 | 0 | 0 |
| THYMU1000240 | 100 | 0 | 0 | 0 | 0 | 0 | 0 |
| THYMU1000242 | 100 | 0 | 0 | 0 | 0 | 0 | 0 |
| THYMU1000291 | 100 | 0 | 0 | 0 | 0 | 0 | 0 |
| THYMU1000316 | 41.147 | 0 | 0 | 0 | 0 | 0 | 0 |
| THYMU1000318 | 100 | 0 | 0 | 0 | 0 | 0 | 0 |
| THYMU1000329 | 4.444 | 0 | 0 | 0 | 0 | 0 | 0 |
| THYMU1000331 | 37.949 | 0 | 0 | 0 | 0 | 0 | 0 |
| THYMU1000336 | 100 | 0 | 0 | 0 | 0 | 0 | 0 |
| THYMU1000359 | 12.068 | 0 | 0 | 0 | 0 | 0 | 0 |
| THYMU1000366 | 100 | 0 | 0 | 0 | 0 | 0 | 0 |
| THYMU1000374 | 11.240 | 0 | 0 | 0 | 0 | 3.355 | 0 |
| THYMU1000382 | 100 | 0 | 0 | 0 | 0 | 0 | 0 |
| THYMU1000393 | 100 | 0 | 0 | 0 | 0 | 0 | 0 |
| THYMU1000394 | 12.297 | 0 | 0 | 0 | 0 | 0 | 0 |
| THYMU1000399 | 100 | 0 | 0 | 0 | 0 | 0 | 0 |
| THYMU1000426 | 100 | 0 | 0 | 0 | 0 | 0 | 0 |
| THYMU1000428 | 0.948 | 2.104 | 0 | 0 | 0 | 0.637 | 0 |
| THYMU1000440 | 100 | 0 | 0 | 0 | 0 | 0 | 0 |
| THYMU1000459 | 100 | 0 | 0 | 0 | 0 | 0 | 0 |
| THYMU1000473 | 2.774 | 0 | 0 | 0 | 0 | 0 | 0 |
| THYMU1000490 | 4.781 | 0 | 0 | 0 | 0 | 6.423 | 0 |
| THYMU1000491 | 100 | 0 | 0 | 0 | 0 | 0 | 0 |
| THYMU1000496 | 100 | 0 | 0 | 0 | 0 | 0 | 0 |
| THYMU1000499 | 4.759 | 0 | 0 | 0 | 0 | 0 | 0 |
| THYMU1000527 | 100 | 0 | 0 | 0 | 0 | 0 | 0 |
| THYMU1000532 | 100 | 0 | 0 | 0 | 0 | 0 | 0 |
| THYMU1000536 | 100 | 0 | 0 | 0 | 0 | 0 | 0 |
| THYMU1000554 | 100 | 0 | 0 | 0 | 0 | 0 | 0 |
| THYMU1000558 | 100 | 0 | 0 | 0 | 0 | 0 | 0 |
| THYMU1000576 | 100 | 0 | 0 | 0 | 0 | 0 | 0 |
| THYMU1000600 | 100 | 0 | 0 | 0 | 0 | 0 | 0 |
| THYMU1000677 | 100 | 0 | 0 | 0 | 0 | 0 | 0 |
| THYMU1000692 | 100 | 0 | 0 | 0 | 0 | 0 | 0 |
| THYMU2000022 | 100 | 0 | 0 | 0 | 0 | 0 | 0 |
| THYMU2000032 | 100 | 0 | 0 | 0 | 0 | 0 | 0 |
| THYMU2000057 | 6.785 | 0 | 18.649 | 0 | 0 | 1.519 | 0 |
| THYMU2000120 | 32.401 | 0 | 0 | 0 | 0 | 0 | 0 |
| THYMU2000138 | 53.005 | 0 | 0 | 0 | 0 | 0 | 0 |
| THYMU2000140 | 100 | 0 | 0 | 0 | 0 | 0 | 0 |
| THYMU2000155 | 42.920 | 0 | 0 | 0 | 0 | 0 | 0 |
| THYMU2000162 | 54.019 | 0 | 0 | 0 | 0 | 0 | 0 |
| THYMU2000171 | 100 | 0 | 0 | 0 | 0 | 0 | 0 |
| THYMU2000176 | 100 | 0 | 0 | 0 | 0 | 0 | 0 |
| THYMU2000179 | 100 | 0 | 0 | 0 | 0 | 0 | 0 |
| THYMU2000231 | 100 | 0 | 0 | 0 | 0 | 0 | 0 |
| THYMU2000236 | 18.303 | 0 | 0 | 0 | 0 | 0 | 0 |
| THYMU2000276 | 100 | 0 | 0 | 0 | 0 | 0 | 0 |
| THYMU2000280 | 100 | 0 | 0 | 0 | 0 | 0 | 0 |
| THYMU2000300 | 73.082 | 0 | 0 | 0 | 0 | 0 | 0 |
| THYMU2000317 | 100 | 0 | 0 | 0 | 0 | 0 | 0 |
| THYMU2000368 | 100 | 0 | 0 | 0 | 0 | 0 | 0 |
| THYMU2000369 | 14.991 | 0 | 0 | 0 | 0 | 0 | 0 |
| THYMU2000382 | 1.516 | 2.244 | 0 | 0 | 13.202 | 0 | 0 |
| THYMU2000418 | 100 | 0 | 0 | 0 | 0 | 0 | 0 |
| THYMU2000436 | 3.842 | 3.411 | 0 | 0 | 0 | 0 | 0 |
| THYMU2000440 | 100 | 0 | 0 | 0 | 0 | 0 | 0 |
| THYMU2000489 | 45.282 | 0 | 0 | 0 | 0 | 0 | 0 |
| THYMU2000492 | 100 | 0 | 0 | 0 | 0 | 0 | 0 |
| THYMU2000519 | 100 | 0 | 0 | 0 | 0 | 0 | 0 |
| THYMU2000570 | 100 | 0 | 0 | 0 | 0 | 0 | 0 |
| THYMU2000602 | 100 | 0 | 0 | 0 | 0 | 0 | 0 |
| THYMU2000660 | 100 | 0 | 0 | 0 | 0 | 0 | 0 |
| THYMU2000671 | 100 | 0 | 0 | 0 | 0 | 0 | 0 |
| THYMU2000672 | 18.861 | 0 | 0 | 0 | 0 | 0 | 0 |
| THYMU2000684 | 6.944 | 15.412 | 0 | 0 | 0 | 0 | 0 |
| THYMU2000702 | 2.609 | 0 | 0 | 0 | 0 | 0 | 0 |
| THYMU2000767 | 100 | 0 | 0 | 0 | 0 | 0 | 0 |
| THYMU2000775 | 15.614 | 0 | 0 | 0 | 0 | 0 | 0 |
| THYMU2000800 | 100 | 0 | 0 | 0 | 0 | 0 | 0 |
| THYMU2000879 | 100 | 0 | 0 | 0 | 0 | 0 | 0 |
| THYMU2000932 | 100 | 0 | 0 | 0 | 0 | 0 | 0 |
| THYMU2000946 | 1.206 | 0 | 0 | 0 | 0 | 0 | 0 |
| THYMU2000950 | 18.385 | 81.615 | 0 | 0 | 0 | 0 | 0 |
| THYMU2000971 | 42.920 | 0 | 0 | 0 | 0 | 0 | 0 |
| THYMU2000982 | 100 | 0 | 0 | 0 | 0 | 0 | 0 |
| THYMU2000990 | 100 | 0 | 0 | 0 | 0 | 0 | 0 |
| THYMU2001007 | 15.835 | 0 | 0 | 0 | 0 | 18.233 | 0 |
| THYMU2001018 | 7.531 | 0 | 0 | 0 | 0 | 0 | 0 |
| THYMU2001053 | 100 | 0 | 0 | 0 | 0 | 0 | 0 |
| THYMU2001071 | 100 | 0 | 0 | 0 | 0 | 0 | 0 |
| THYMU2001090 | 17.721 | 0 | 0 | 0 | 0 | 0 | 0 |
| THYMU2001139 | 100 | 0 | 0 | 0 | 0 | 0 | 0 |
| THYMU2001195 | 32.401 | 0 | 0 | 0 | 0 | 0 | 0 |
| THYMU2001202 | 100 | 0 | 0 | 0 | 0 | 0 | 0 |
| THYMU2001203 | 100 | 0 | 0 | 0 | 0 | 0 | 0 |
| THYMU2001256 | 100 | 0 | 0 | 0 | 0 | 0 | 0 |
| THYMU2001270 | 100 | 0 | 0 | 0 | 0 | 0 | 0 |
| THYMU2001291 | 100 | 0 | 0 | 0 | 0 | 0 | 0 |
| THYMU2001314 | 100 | 0 | 0 | 0 | 0 | 0 | 0 |
| THYMU2001325 | 100 | 0 | 0 | 0 | 0 | 0 | 0 |
| THYMU2001357 | 100 | 0 | 0 | 0 | 0 | 0 | 0 |
| THYMU2001381 | 5.656 | 0 | 46.631 | 0 | 0 | 0 | 0 |
| THYMU2001422 | 10.386 | 0 | 0 | 0 | 0 | 0 | 0 |
| THYMU2001443 | 100 | 0 | 0 | 0 | 0 | 0 | 0 |
| THYMU2001515 | 85.122 | 0 | 0 | 0 | 0 | 0 | 0 |
| THYMU2001521 | 16.572 | 0 | 0 | 0 | 0 | 0 | 0 |
| THYMU2001556 | 100 | 0 | 0 | 0 | 0 | 0 | 0 |
| THYMU2001622 | 100 | 0 | 0 | 0 | 0 | 0 | 0 |
| THYMU2001727 | 100 | 0 | 0 | 0 | 0 | 0 | 0 |
| THYMU2001819 | 19.114 | 0 | 0 | 0 | 0 | 0 | 0 |
| THYMU2001825 | 100 | 0 | 0 | 0 | 0 | 0 | 0 |
| THYMU2001839 | 13.046 | 0 | 0 | 0 | 0 | 0 | 0 |
| THYMU2001868 | 26.657 | 0 | 0 | 0 | 0 | 0 | 0 |
| THYMU2001898 | 100 | 0 | 0 | 0 | 0 | 0 | 0 |
| THYMU2001900 | 100 | 0 | 0 | 0 | 0 | 0 | 0 |
| THYMU2001916 | 100 | 0 | 0 | 0 | 0 | 0 | 0 |
| THYMU2001926 | 16.184 | 0 | 0 | 0 | 0 | 0 | 0 |
| THYMU2001989 | 100 | 0 | 0 | 0 | 0 | 0 | 0 |
| THYMU2002023 | 22.349 | 0 | 0 | 0 | 0 | 0 | 0 |
| THYMU2002037 | 100 | 0 | 0 | 0 | 0 | 0 | 0 |
| THYMU2002080 | 12.409 | 0 | 0 | 0 | 0 | 0 | 0 |
| THYMU2002109 | 100 | 0 | 0 | 0 | 0 | 0 | 0 |
| THYMU2002147 | 58.982 | 0 | 0 | 0 | 0 | 0 | 0 |
| THYMU2002154 | 100 | 0 | 0 | 0 | 0 | 0 | 0 |
| THYMU2002157 | 100 | 0 | 0 | 0 | 0 | 0 | 0 |
| THYMU2002162 | 18.182 | 0 | 0 | 0 | 0 | 0 | 0 |
| THYMU2002177 | 100 | 0 | 0 | 0 | 0 | 0 | 0 |
| THYMU2002203 | 100 | 0 | 0 | 0 | 0 | 0 | 0 |
| THYMU2002229 | 18.158 | 0 | 0 | 0 | 0 | 0 | 0 |
| THYMU2002242 | 100 | 0 | 0 | 0 | 0 | 0 | 0 |
| THYMU2002338 | 100 | 0 | 0 | 0 | 0 | 0 | 0 |
| THYMU2002356 | 100 | 0 | 0 | 0 | 0 | 0 | 0 |
| THYMU2002376 | 100 | 0 | 0 | 0 | 0 | 0 | 0 |
| THYMU2002384 | 100 | 0 | 0 | 0 | 0 | 0 | 0 |
| THYMU2002450 | 35.978 | 0 | 0 | 0 | 0 | 24.165 | 0 |
| THYMU2002490 | 100 | 0 | 0 | 0 | 0 | 0 | 0 |
| THYMU2002548 | 100 | 0 | 0 | 0 | 0 | 0 | 0 |
| THYMU2002583 | 2.161 | 1.918 | 3.563 | 0 | 11.289 | 2.322 | 0 |
| THYMU2002695 | 100 | 0 | 0 | 0 | 0 | 0 | 0 |
| THYMU2002745 | 3.192 | 0 | 0 | 0 | 0 | 0 | 0 |
| THYMU2002756 | 12.209 | 0 | 0 | 0 | 0 | 8.200 | 0 |
| THYMU2002789 | 100 | 0 | 0 | 0 | 0 | 0 | 0 |
| THYMU2002792 | 100 | 0 | 0 | 0 | 0 | 0 | 0 |
| THYMU2002815 | 27.551 | 0 | 0 | 0 | 0 | 0 | 0 |
| THYMU2002841 | 4.840 | 0 | 0 | 0 | 0 | 0 | 0 |
| THYMU2002852 | 100 | 0 | 0 | 0 | 0 | 0 | 0 |
| THYMU2002910 | 100 | 0 | 0 | 0 | 0 | 0 | 0 |
| THYMU2002950 | 100 | 0 | 0 | 0 | 0 | 0 | 0 |
| THYMU2002983 | 2.495 | 0 | 0 | 0 | 0 | 0 | 0 |
| THYMU2003012 | 1.461 | 0 | 0 | 0 | 0 | 0.982 | 0 |
| THYMU2003046 | 20.838 | 0 | 0 | 0 | 0 | 0 | 0 |
| THYMU2003069 | 92.134 | 0 | 0 | 0 | 0 | 0 | 0 |
| THYMU2003107 | 100 | 0 | 0 | 0 | 0 | 0 | 0 |
| THYMU2003124 | 100 | 0 | 0 | 0 | 0 | 0 | 0 |
| THYMU2003133 | 14.465 | 0 | 0 | 0 | 0 | 3.886 | 0 |
| THYMU2003166 | 100 | 0 | 0 | 0 | 0 | 0 | 0 |
| THYMU2003232 | 100 | 0 | 0 | 0 | 0 | 0 | 0 |
| THYMU2003242 | 100 | 0 | 0 | 0 | 0 | 0 | 0 |
| THYMU2003249 | 100 | 0 | 0 | 0 | 0 | 0 | 0 |
| THYMU2003270 | 100 | 0 | 0 | 0 | 0 | 0 | 0 |
| THYMU2003282 | 100 | 0 | 0 | 0 | 0 | 0 | 0 |
| THYMU2003287 | 79.316 | 0 | 0 | 0 | 0 | 0 | 0 |
| THYMU2003336 | 100 | 0 | 0 | 0 | 0 | 0 | 0 |
| THYMU2003366 | 100 | 0 | 0 | 0 | 0 | 0 | 0 |
| THYMU2003397 | 100 | 0 | 0 | 0 | 0 | 0 | 0 |
| THYMU2003419 | 6.700 | 0 | 0 | 0 | 0 | 2.250 | 0 |
| THYMU2003440 | 19.399 | 0 | 0 | 0 | 0 | 0 | 0 |
| THYMU2003446 | 5.966 | 0 | 0 | 0 | 0 | 0 | 0 |
| THYMU2003470 | 100 | 0 | 0 | 0 | 0 | 0 | 0 |
| THYMU2003479 | 100 | 0 | 0 | 0 | 0 | 0 | 0 |
| THYMU2003499 | 37.705 | 0 | 0 | 0 | 0 | 0 | 0 |
| THYMU2003542 | 1.802 | 0 | 0 | 0 | 0 | 0 | 0 |
| THYMU2003632 | 100 | 0 | 0 | 0 | 0 | 0 | 0 |
| THYMU2003650 | 100 | 0 | 0 | 0 | 0 | 0 | 0 |
| THYMU2003700 | 100 | 0 | 0 | 0 | 0 | 0 | 0 |
| THYMU2003760 | 100 | 0 | 0 | 0 | 0 | 0 | 0 |
| THYMU2003803 | 100 | 0 | 0 | 0 | 0 | 0 | 0 |
| THYMU2003855 | 100 | 0 | 0 | 0 | 0 | 0 | 0 |
| THYMU2003891 | 4.380 | 0 | 0 | 0 | 0 | 0.163 | 3.134 |
| THYMU2003907 | 9.456 | 0 | 77.967 | 0 | 0 | 0 | 0 |
| THYMU2003932 | 32.257 | 0 | 0 | 0 | 0 | 0 | 0 |
| THYMU2003981 | 2.613 | 0 | 0 | 0 | 0 | 3.510 | 11.216 |
| THYMU2004003 | 100 | 0 | 0 | 0 | 0 | 0 | 0 |
| THYMU2004008 | 100 | 0 | 0 | 0 | 0 | 0 | 0 |
| THYMU2004042 | 100 | 0 | 0 | 0 | 0 | 0 | 0 |
| THYMU2004108 | 11.015 | 0 | 0 | 0 | 0 | 0 | 0 |
| THYMU2004111 | 100 | 0 | 0 | 0 | 0 | 0 | 0 |
| THYMU2004123 | 100 | 0 | 0 | 0 | 0 | 0 | 0 |
| THYMU2004139 | 100 | 0 | 0 | 0 | 0 | 0 | 0 |
| THYMU2004152 | 9.107 | 0 | 0 | 0 | 0 | 0 | 21.324 |
| THYMU2004169 | 100 | 0 | 0 | 0 | 0 | 0 | 0 |
| THYMU2004284 | 5.601 | 0 | 0 | 0 | 0 | 0 | 0 |
| THYMU2004320 | 13.625 | 0 | 0 | 0 | 0 | 0 | 0 |
| THYMU2004336 | 100 | 0 | 0 | 0 | 0 | 0 | 0 |
| THYMU2004344 | 2.130 | 0 | 0 | 0 | 0 | 0 | 0 |
| THYMU2004356 | 100 | 0 | 0 | 0 | 0 | 0 | 0 |
| THYMU2004410 | 3.195 | 0 | 0 | 0 | 0 | 2.146 | 0 |
| THYMU2004452 | 19.272 | 0 | 0 | 0 | 0 | 0 | 0 |
| THYMU2004508 | 54.019 | 0 | 0 | 0 | 0 | 0 | 0 |
| THYMU2004512 | 100 | 0 | 0 | 0 | 0 | 0 | 0 |
| THYMU2004635 | 44.974 | 0 | 0 | 0 | 0 | 0 | 0 |
| THYMU2004639 | 100 | 0 | 0 | 0 | 0 | 0 | 0 |
| THYMU2004668 | 100 | 0 | 0 | 0 | 0 | 0 | 0 |
| THYMU2004677 | 100 | 0 | 0 | 0 | 0 | 0 | 0 |
| THYMU2004688 | 100 | 0 | 0 | 0 | 0 | 0 | 0 |
| THYMU2004693 | 100 | 0 | 0 | 0 | 0 | 0 | 0 |
| THYMU2004733 | 9.340 | 0 | 0 | 0 | 0 | 0 | 0 |
| THYMU2004835 | 100 | 0 | 0 | 0 | 0 | 0 | 0 |
| THYMU2004843 | 100 | 0 | 0 | 0 | 0 | 0 | 0 |
| THYMU2004906 | 10.451 | 0 | 0 | 0 | 0 | 0 | 0 |
| THYMU2004916 | 2.602 | 0 | 0 | 0 | 33.990 | 0 | 0 |
| THYMU2004986 | 100 | 0 | 0 | 0 | 0 | 0 | 0 |
| THYMU2005001 | 100 | 0 | 0 | 0 | 0 | 0 | 0 |
| THYMU2005003 | 11.644 | 0 | 0 | 0 | 0 | 0 | 0 |
| THYMU2005046 | 100 | 0 | 0 | 0 | 0 | 0 | 0 |
| THYMU2005134 | 100 | 0 | 0 | 0 | 0 | 0 | 0 |
| THYMU2005156 | 31.905 | 0 | 0 | 0 | 0 | 0 | 0 |
| THYMU2005190 | 100 | 0 | 0 | 0 | 0 | 0 | 0 |
| THYMU2005225 | 100 | 0 | 0 | 0 | 0 | 0 | 0 |
| THYMU2005240 | 100 | 0 | 0 | 0 | 0 | 0 | 0 |
| THYMU2005246 | 42.674 | 0 | 0 | 0 | 0 | 57.326 | 0 |
| THYMU2005270 | 100 | 0 | 0 | 0 | 0 | 0 | 0 |
| THYMU2005283 | 100 | 0 | 0 | 0 | 0 | 0 | 0 |
| THYMU2005303 | 100 | 0 | 0 | 0 | 0 | 0 | 0 |
| THYMU2005321 | 100 | 0 | 0 | 0 | 0 | 0 | 0 |
| THYMU2005356 | 100 | 0 | 0 | 0 | 0 | 0 | 0 |
| THYMU2005369 | 100 | 0 | 0 | 0 | 0 | 0 | 0 |
| THYMU2005439 | 13.827 | 0 | 0 | 0 | 0 | 0 | 0 |
| THYMU2005480 | 100 | 0 | 0 | 0 | 0 | 0 | 0 |
| THYMU2005482 | 10.260 | 0 | 0 | 0 | 0 | 0 | 0 |
| THYMU2005507 | 100 | 0 | 0 | 0 | 0 | 0 | 0 |
| THYMU2005545 | 100 | 0 | 0 | 0 | 0 | 0 | 0 |
| THYMU2005546 | 2.737 | 0 | 0 | 0 | 0 | 0.613 | 0 |
| THYMU2005574 | 54.019 | 0 | 0 | 0 | 0 | 0 | 0 |
| THYMU2005576 | 100 | 0 | 0 | 0 | 0 | 0 | 0 |
| THYMU2005748 | 100 | 0 | 0 | 0 | 0 | 0 | 0 |
| THYMU2005759 | 100 | 0 | 0 | 0 | 0 | 0 | 0 |
| THYMU2005807 | 54.019 | 0 | 0 | 0 | 0 | 0 | 0 |
| THYMU2005815 | 100 | 0 | 0 | 0 | 0 | 0 | 0 |
| THYMU2005855 | 100 | 0 | 0 | 0 | 0 | 0 | 0 |
| THYMU2005881 | 100 | 0 | 0 | 0 | 0 | 0 | 0 |
| THYMU2005948 | 100 | 0 | 0 | 0 | 0 | 0 | 0 |
| THYMU2005980 | 6.762 | 0 | 0 | 0 | 0 | 0 | 0 |
| THYMU2005997 | 26.854 | 0 | 0 | 0 | 0 | 0 | 0 |
| THYMU2006001 | 100 | 0 | 0 | 0 | 0 | 0 | 0 |
| THYMU2006048 | 100 | 0 | 0 | 0 | 0 | 0 | 0 |
| THYMU2006098 | 100 | 0 | 0 | 0 | 0 | 0 | 0 |
| THYMU2006160 | 100 | 0 | 0 | 0 | 0 | 0 | 0 |
| THYMU2006170 | 100 | 0 | 0 | 0 | 0 | 0 | 0 |
| THYMU2006252 | 100 | 0 | 0 | 0 | 0 | 0 | 0 |
| THYMU2006261 | 24.179 | 0 | 0 | 0 | 0 | 0 | 0 |
| THYMU2006277 | 0.967 | 0 | 0 | 0 | 0 | 0 | 0 |
| THYMU2006303 | 100 | 0 | 0 | 0 | 0 | 0 | 0 |
| THYMU2006324 | 100 | 0 | 0 | 0 | 0 | 0 | 0 |
| THYMU2006350 | 100 | 0 | 0 | 0 | 0 | 0 | 0 |
| THYMU2006357 | 100 | 0 | 0 | 0 | 0 | 0 | 0 |
| THYMU2006365 | 100 | 0 | 0 | 0 | 0 | 0 | 0 |
| THYMU2006420 | 100 | 0 | 0 | 0 | 0 | 0 | 0 |
| THYMU2006468 | 100 | 0 | 0 | 0 | 0 | 0 | 0 |
| THYMU2006505 | 2.277 | 0 | 0 | 0 | 0 | 0 | 0 |
| THYMU2006610 | 22.644 | 0 | 0 | 0 | 0 | 0 | 0 |
| THYMU2006666 | 100 | 0 | 0 | 0 | 0 | 0 | 0 |
| THYMU2006712 | 100 | 0 | 0 | 0 | 0 | 0 | 0 |
| THYMU2006813 | 9.236 | 0 | 0 | 0 | 0 | 3.545 | 0 |
| THYMU2006913 | 100 | 0 | 0 | 0 | 0 | 0 | 0 |
| THYMU2006946 | 100 | 0 | 0 | 0 | 0 | 0 | 0 |
| THYMU2006965 | 100 | 0 | 0 | 0 | 0 | 0 | 0 |
| THYMU2007025 | 100 | 0 | 0 | 0 | 0 | 0 | 0 |
| THYMU2007036 | 6.438 | 0 | 0 | 0 | 0 | 0 | 0 |
| THYMU2007060 | 7.110 | 0 | 0 | 0 | 0 | 0 | 0 |
| THYMU2007065 | 10.144 | 0 | 0 | 0 | 0 | 0 | 0 |
| THYMU2007095 | 100 | 0 | 0 | 0 | 0 | 0 | 0 |
| THYMU2007112 | 3.813 | 0 | 0 | 0 | 0 | 0 | 0 |
| THYMU2007146 | 29.835 | 0 | 0 | 0 | 0 | 0 | 0 |
| THYMU2007179 | 100 | 0 | 0 | 0 | 0 | 0 | 0 |
| THYMU2007258 | 100 | 0 | 0 | 0 | 0 | 0 | 0 |
| THYMU2007307 | 100 | 0 | 0 | 0 | 0 | 0 | 0 |
| THYMU2007308 | 100 | 0 | 0 | 0 | 0 | 0 | 0 |
| THYMU2007339 | 25.705 | 0 | 0 | 0 | 0 | 0 | 0 |
| THYMU2007350 | 100 | 0 | 0 | 0 | 0 | 0 | 0 |
| THYMU2007384 | 100 | 0 | 0 | 0 | 0 | 0 | 0 |
| THYMU2007415 | 8.071 | 0 | 0 | 0 | 0 | 0 | 0 |
| THYMU2007467 | 6.736 | 0 | 0 | 0 | 0 | 0 | 0 |
| THYMU2007493 | 100 | 0 | 0 | 0 | 0 | 0 | 0 |
| THYMU2007521 | 100 | 0 | 0 | 0 | 0 | 0 | 0 |
| THYMU2007528 | 100 | 0 | 0 | 0 | 0 | 0 | 0 |
| THYMU2007530 | 100 | 0 | 0 | 0 | 0 | 0 | 0 |
| THYMU2007532 | 26.053 | 0 | 0 | 0 | 0 | 17.499 | 0 |
| THYMU2007561 | 100 | 0 | 0 | 0 | 0 | 0 | 0 |
| THYMU2007571 | 100 | 0 | 0 | 0 | 0 | 0 | 0 |
| THYMU2007635 | 100 | 0 | 0 | 0 | 0 | 0 | 0 |
| THYMU2007658 | 100 | 0 | 0 | 0 | 0 | 0 | 0 |
| THYMU2007667 | 100 | 0 | 0 | 0 | 0 | 0 | 0 |
| THYMU2007694 | 17.288 | 0 | 0 | 0 | 0 | 0 | 0 |
| THYMU2007725 | 100 | 0 | 0 | 0 | 0 | 0 | 0 |
| THYMU2007729 | 8.151 | 0 | 0 | 0 | 0 | 0 | 0 |
| THYMU2007802 | 25.065 | 0 | 0 | 0 | 0 | 0 | 0 |
| THYMU2007824 | 100 | 0 | 0 | 0 | 0 | 0 | 0 |
| THYMU2007854 | 100 | 0 | 0 | 0 | 0 | 0 | 0 |
| THYMU2007886 | 1.155 | 0 | 0 | 0 | 0 | 0 | 0 |
| THYMU2007952 | 100 | 0 | 0 | 0 | 0 | 0 | 0 |
| THYMU2007969 | 100 | 0 | 0 | 0 | 0 | 0 | 0 |
| THYMU2008035 | 45.470 | 0 | 0 | 0 | 0 | 0 | 0 |
| THYMU2008036 | 45.282 | 0 | 0 | 0 | 0 | 0 | 0 |
| THYMU2008049 | 18.834 | 0 | 0 | 0 | 0 | 0 | 0 |
| THYMU2008072 | 100 | 0 | 0 | 0 | 0 | 0 | 0 |
| THYMU2008111 | 100 | 0 | 0 | 0 | 0 | 0 | 0 |
| THYMU2008145 | 100 | 0 | 0 | 0 | 0 | 0 | 0 |
| THYMU2008149 | 73.674 | 0 | 0 | 0 | 0 | 19.794 | 0 |
| THYMU2008185 | 2.405 | 0 | 0 | 0 | 0 | 0 | 0 |
| THYMU2008191 | 100 | 0 | 0 | 0 | 0 | 0 | 0 |
| THYMU2008207 | 100 | 0 | 0 | 0 | 0 | 0 | 0 |
| THYMU2008226 | 100 | 0 | 0 | 0 | 0 | 0 | 0 |
| THYMU2008282 | 100 | 0 | 0 | 0 | 0 | 0 | 0 |
| THYMU2008321 | 2.411 | 0 | 0 | 0 | 0 | 0 | 0 |
| THYMU2008339 | 100 | 0 | 0 | 0 | 0 | 0 | 0 |
| THYMU2008350 | 100 | 0 | 0 | 0 | 0 | 0 | 0 |
| THYMU2008351 | 100 | 0 | 0 | 0 | 0 | 0 | 0 |
| THYMU2008383 | 56.056 | 0 | 0 | 0 | 0 | 0 | 0 |
| THYMU2008452 | 100 | 0 | 0 | 0 | 0 | 0 | 0 |
| THYMU2008643 | 100 | 0 | 0 | 0 | 0 | 0 | 0 |
| THYMU2008686 | 20.324 | 0 | 0 | 0 | 0 | 0 | 0 |
| THYMU2008691 | 100 | 0 | 0 | 0 | 0 | 0 | 0 |
| THYMU2008714 | 100 | 0 | 0 | 0 | 0 | 0 | 0 |
| THYMU2008725 | 10.873 | 0 | 0 | 0 | 0 | 0 | 0 |
| THYMU2008727 | 12.395 | 0 | 0 | 0 | 0 | 0 | 0 |
| THYMU2008781 | 100 | 0 | 0 | 0 | 0 | 0 | 0 |
| THYMU2008799 | 100 | 0 | 0 | 0 | 0 | 0 | 0 |
| THYMU2008876 | 100 | 0 | 0 | 0 | 0 | 0 | 0 |
| THYMU2008910 | 100 | 0 | 0 | 0 | 0 | 0 | 0 |
| THYMU2008917 | 100 | 0 | 0 | 0 | 0 | 0 | 0 |
| THYMU2008928 | 100 | 0 | 0 | 0 | 0 | 0 | 0 |
| THYMU2008990 | 100 | 0 | 0 | 0 | 0 | 0 | 0 |
| THYMU2008994 | 100 | 0 | 0 | 0 | 0 | 0 | 0 |
| THYMU2009023 | 100 | 0 | 0 | 0 | 0 | 0 | 0 |
| THYMU2009046 | 100 | 0 | 0 | 0 | 0 | 0 | 0 |
| THYMU2009101 | 100 | 0 | 0 | 0 | 0 | 0 | 0 |
| THYMU2009104 | 100 | 0 | 0 | 0 | 0 | 0 | 0 |
| THYMU2009134 | 100 | 0 | 0 | 0 | 0 | 0 | 0 |
| THYMU2009157 | 100 | 0 | 0 | 0 | 0 | 0 | 0 |
| THYMU2009181 | 100 | 0 | 0 | 0 | 0 | 0 | 0 |
| THYMU2009186 | 32.401 | 0 | 0 | 0 | 0 | 0 | 0 |
| THYMU2009338 | 42.920 | 0 | 0 | 0 | 0 | 0 | 0 |
| THYMU2009425 | 100 | 0 | 0 | 0 | 0 | 0 | 0 |
| THYMU2009516 | 100 | 0 | 0 | 0 | 0 | 0 | 0 |
| THYMU2009526 | 100 | 0 | 0 | 0 | 0 | 0 | 0 |
| THYMU2009546 | 100 | 0 | 0 | 0 | 0 | 0 | 0 |
| THYMU2009592 | 46.072 | 0 | 0 | 0 | 0 | 0 | 0 |
| THYMU2009596 | 100 | 0 | 0 | 0 | 0 | 0 | 0 |
| THYMU2009658 | 0.584 | 0 | 0 | 0 | 0 | 0.392 | 7.523 |
| THYMU2009792 | 2.335 | 0 | 0 | 0 | 0 | 0 | 0 |
| THYMU2009835 | 100 | 0 | 0 | 0 | 0 | 0 | 0 |
| THYMU2009906 | 100 | 0 | 0 | 0 | 0 | 0 | 0 |
| THYMU2009948 | 100 | 0 | 0 | 0 | 0 | 0 | 0 |
| THYMU2010030 | 100 | 0 | 0 | 0 | 0 | 0 | 0 |
| THYMU2010041 | 62.190 | 0 | 0 | 0 | 0 | 0 | 0 |
| THYMU2010046 | 100 | 0 | 0 | 0 | 0 | 0 | 0 |
| THYMU2010059 | 100 | 0 | 0 | 0 | 0 | 0 | 0 |
| THYMU2010082 | 100 | 0 | 0 | 0 | 0 | 0 | 0 |
| THYMU2010094 | 24.284 | 0 | 0 | 0 | 0 | 6.524 | 0 |
| THYMU2010161 | 12.580 | 0 | 0 | 0 | 0 | 0 | 0 |
| THYMU2010192 | 42.920 | 0 | 0 | 0 | 0 | 0 | 0 |
| THYMU2010448 | 4.415 | 0 | 0 | 0 | 0 | 0 | 0 |
| THYMU2010519 | 86.171 | 0 | 0 | 0 | 0 | 0 | 0 |
| THYMU2010637 | 100 | 0 | 0 | 0 | 0 | 0 | 0 |
| THYMU2010671 | 100 | 0 | 0 | 0 | 0 | 0 | 0 |
| THYMU2010699 | 100 | 0 | 0 | 0 | 0 | 0 | 0 |
| THYMU2010705 | 100 | 0 | 0 | 0 | 0 | 0 | 0 |
| THYMU2010779 | 70.146 | 0 | 0 | 0 | 0 | 0 | 0 |
| THYMU2010816 | 10.870 | 48.256 | 0 | 0 | 0 | 14.603 | 0 |
| THYMU2010831 | 45.470 | 0 | 0 | 0 | 0 | 0 | 0 |
| THYMU2010848 | 100 | 0 | 0 | 0 | 0 | 0 | 0 |
| THYMU2011053 | 42.674 | 0 | 0 | 0 | 0 | 57.326 | 0 |
| THYMU2011063 | 4.388 | 0 | 0 | 0 | 0 | 0 | 0 |
| THYMU2011072 | 100 | 0 | 0 | 0 | 0 | 0 | 0 |
| THYMU2011096 | 100 | 0 | 0 | 0 | 0 | 0 | 0 |
| THYMU2011118 | 100 | 0 | 0 | 0 | 0 | 0 | 0 |
| THYMU2011142 | 100 | 0 | 0 | 0 | 0 | 0 | 0 |
| THYMU2011183 | 100 | 0 | 0 | 0 | 0 | 0 | 0 |
| THYMU2011257 | 100 | 0 | 0 | 0 | 0 | 0 | 0 |
| THYMU2011259 | 100 | 0 | 0 | 0 | 0 | 0 | 0 |
| THYMU2011295 | 100 | 0 | 0 | 0 | 0 | 0 | 0 |
| THYMU2011308 | 7.001 | 0 | 0 | 0 | 0 | 0 | 0 |
| THYMU2011390 | 100 | 0 | 0 | 0 | 0 | 0 | 0 |
| THYMU2011431 | 100 | 0 | 0 | 0 | 0 | 0 | 0 |
| THYMU2011447 | 100 | 0 | 0 | 0 | 0 | 0 | 0 |
| THYMU2011482 | 10.238 | 0 | 0 | 0 | 0 | 0 | 0 |
| THYMU2011538 | 100 | 0 | 0 | 0 | 0 | 0 | 0 |
| THYMU2011548 | 100 | 0 | 0 | 0 | 0 | 0 | 0 |
| THYMU2011551 | 100 | 0 | 0 | 0 | 0 | 0 | 0 |
| THYMU2011564 | 100 | 0 | 0 | 0 | 0 | 0 | 0 |
| THYMU2011573 | 100 | 0 | 0 | 0 | 0 | 0 | 0 |
| THYMU2011585 | 100 | 0 | 0 | 0 | 0 | 0 | 0 |
| THYMU2011621 | 100 | 0 | 0 | 0 | 0 | 0 | 0 |
| THYMU2011666 | 100 | 0 | 0 | 0 | 0 | 0 | 0 |
| THYMU2011736 | 25.065 | 0 | 0 | 0 | 0 | 0 | 0 |
| THYMU2011785 | 100 | 0 | 0 | 0 | 0 | 0 | 0 |
| THYMU2011806 | 100 | 0 | 0 | 0 | 0 | 0 | 0 |
| THYMU2011852 | 100 | 0 | 0 | 0 | 0 | 0 | 0 |
| THYMU2011875 | 18.705 | 0 | 0 | 0 | 0 | 0 | 0 |
| THYMU2011881 | 8.692 | 0 | 0 | 0 | 0 | 0 | 0 |
| THYMU2011937 | 100 | 0 | 0 | 0 | 0 | 0 | 0 |
| THYMU2011939 | 54.019 | 0 | 0 | 0 | 0 | 0 | 0 |
| THYMU2012002 | 21.171 | 0 | 0 | 0 | 0 | 0 | 0 |
| THYMU2012024 | 9.940 | 0 | 0 | 0 | 0 | 3.338 | 0 |
| THYMU2012073 | 100 | 0 | 0 | 0 | 0 | 0 | 0 |
| THYMU2012104 | 22.558 | 0 | 0 | 0 | 0 | 0 | 0 |
| THYMU2012113 | 100 | 0 | 0 | 0 | 0 | 0 | 0 |
| THYMU2012273 | 100 | 0 | 0 | 0 | 0 | 0 | 0 |
| THYMU2012401 | 19.301 | 0 | 0 | 0 | 0 | 0 | 0 |
| THYMU2012492 | 54.510 | 0 | 0 | 0 | 0 | 0 | 0 |
| THYMU2012507 | 100 | 0 | 0 | 0 | 0 | 0 | 0 |
| THYMU2012565 | 100 | 0 | 0 | 0 | 0 | 0 | 0 |
| THYMU2012574 | 100 | 0 | 0 | 0 | 0 | 0 | 0 |
| THYMU2012625 | 2.599 | 0 | 0 | 0 | 0 | 0 | 22.310 |
| THYMU2012631 | 100 | 0 | 0 | 0 | 0 | 0 | 0 |
| THYMU2012665 | 100 | 0 | 0 | 0 | 0 | 0 | 0 |
| THYMU2012690 | 3.240 | 0 | 0 | 0 | 0 | 0 | 0 |
| THYMU2012701 | 100 | 0 | 0 | 0 | 0 | 0 | 0 |
| THYMU2012807 | 100 | 0 | 0 | 0 | 0 | 0 | 0 |
| THYMU2012826 | 18.467 | 0 | 0 | 0 | 0 | 0 | 0 |
| THYMU2012882 | 100 | 0 | 0 | 0 | 0 | 0 | 0 |
| THYMU2012891 | 100 | 0 | 0 | 0 | 0 | 0 | 0 |
| THYMU2012902 | 100 | 0 | 0 | 0 | 0 | 0 | 0 |
| THYMU2012919 | 100 | 0 | 0 | 0 | 0 | 0 | 0 |
| THYMU2013023 | 100 | 0 | 0 | 0 | 0 | 0 | 0 |
| THYMU2013047 | 100 | 0 | 0 | 0 | 0 | 0 | 0 |
| THYMU2013051 | 18.782 | 0 | 0 | 0 | 0 | 0 | 0 |
| THYMU2013089 | 100 | 0 | 0 | 0 | 0 | 0 | 0 |
| THYMU2013136 | 2.046 | 0 | 0 | 0 | 0 | 2.748 | 0 |
| THYMU2013170 | 19.114 | 0 | 0 | 0 | 0 | 0 | 0 |
| THYMU2013364 | 100 | 0 | 0 | 0 | 0 | 0 | 0 |
| THYMU2013386 | 100 | 0 | 0 | 0 | 0 | 0 | 0 |
| THYMU2013426 | 100 | 0 | 0 | 0 | 0 | 0 | 0 |
| THYMU2013578 | 100 | 0 | 0 | 0 | 0 | 0 | 0 |
| THYMU2013648 | 9.986 | 0 | 0 | 0 | 0 | 0 | 0 |
| THYMU2013705 | 100 | 0 | 0 | 0 | 0 | 0 | 0 |
| THYMU2013757 | 100 | 0 | 0 | 0 | 0 | 0 | 0 |
| THYMU2013779 | 100 | 0 | 0 | 0 | 0 | 0 | 0 |
| THYMU2013863 | 100 | 0 | 0 | 0 | 0 | 0 | 0 |
| THYMU2013916 | 0.925 | 8.211 | 0 | 0 | 0 | 1.242 | 0 |
| THYMU2013950 | 100 | 0 | 0 | 0 | 0 | 0 | 0 |
| THYMU2013981 | 100 | 0 | 0 | 0 | 0 | 0 | 0 |
| THYMU2014067 | 12.580 | 0 | 0 | 0 | 0 | 0 | 0 |
| THYMU2014068 | 100 | 0 | 0 | 0 | 0 | 0 | 0 |
| THYMU2014167 | 4.582 | 0 | 0 | 0 | 0 | 0 | 0 |
| THYMU2014183 | 16.202 | 0 | 0 | 0 | 0 | 0 | 0 |
| THYMU2014204 | 100 | 0 | 0 | 0 | 0 | 0 | 0 |
| THYMU2014297 | 100 | 0 | 0 | 0 | 0 | 0 | 0 |
| THYMU2014323 | 45.282 | 0 | 0 | 0 | 0 | 0 | 0 |
| THYMU2014327 | 100 | 0 | 0 | 0 | 0 | 0 | 0 |
| THYMU2014353 | 62.337 | 0 | 0 | 0 | 0 | 0 | 0 |
| THYMU2014492 | 32.093 | 0 | 0 | 0 | 0 | 0 | 0 |
| THYMU2014500 | 100 | 0 | 0 | 0 | 0 | 0 | 0 |
| THYMU2014599 | 47.240 | 0 | 0 | 0 | 0 | 0 | 0 |
| THYMU2014762 | 100 | 0 | 0 | 0 | 0 | 0 | 0 |
| THYMU2014777 | 100 | 0 | 0 | 0 | 0 | 0 | 0 |
| THYMU2014801 | 100 | 0 | 0 | 0 | 0 | 0 | 0 |
| THYMU2014901 | 100 | 0 | 0 | 0 | 0 | 0 | 0 |
| THYMU2014923 | 100 | 0 | 0 | 0 | 0 | 0 | 0 |
| THYMU2015019 | 22.813 | 0 | 0 | 0 | 0 | 0 | 0 |
| THYMU2015161 | 100 | 0 | 0 | 0 | 0 | 0 | 0 |
| THYMU2015316 | 100 | 0 | 0 | 0 | 0 | 0 | 0 |
| THYMU2015321 | 41.147 | 0 | 0 | 0 | 0 | 0 | 0 |
| THYMU2015439 | 100 | 0 | 0 | 0 | 0 | 0 | 0 |
| THYMU2015441 | 18.750 | 0 | 0 | 0 | 0 | 0 | 0 |
| THYMU2015479 | 100 | 0 | 0 | 0 | 0 | 0 | 0 |
| THYMU2015762 | 0.827 | 3.670 | 0 | 0 | 0 | 0 | 0 |
| THYMU2015775 | 18.523 | 0 | 0 | 0 | 0 | 0 | 0 |
| THYMU2015825 | 100 | 0 | 0 | 0 | 0 | 0 | 0 |
| THYMU2015918 | 44.974 | 0 | 0 | 0 | 0 | 0 | 0 |
| THYMU2015943 | 100 | 0 | 0 | 0 | 0 | 0 | 0 |
| THYMU2015984 | 100 | 0 | 0 | 0 | 0 | 0 | 0 |
| THYMU2016113 | 100 | 0 | 0 | 0 | 0 | 0 | 0 |
| THYMU2016164 | 100 | 0 | 0 | 0 | 0 | 0 | 0 |
| THYMU2016204 | 40.164 | 0 | 0 | 0 | 0 | 0 | 0 |
| THYMU2016219 | 100 | 0 | 0 | 0 | 0 | 0 | 0 |
| THYMU2016360 | 100 | 0 | 0 | 0 | 0 | 0 | 0 |
| THYMU2016496 | 100 | 0 | 0 | 0 | 0 | 0 | 0 |
| THYMU2016523 | 55.019 | 0 | 0 | 0 | 0 | 0 | 0 |
| THYMU2016840 | 18.750 | 0 | 0 | 0 | 0 | 0 | 0 |
| THYMU2016968 | 6.163 | 0 | 0 | 0 | 0 | 0 | 0 |
| THYMU2017008 | 100 | 0 | 0 | 0 | 0 | 0 | 0 |
| THYMU2017010 | 100 | 0 | 0 | 0 | 0 | 0 | 0 |
| THYMU2017023 | 100 | 0 | 0 | 0 | 0 | 0 | 0 |
| THYMU2017085 | 100 | 0 | 0 | 0 | 0 | 0 | 0 |
| THYMU2017099 | 100 | 0 | 0 | 0 | 0 | 0 | 0 |
| THYMU2017158 | 40.164 | 0 | 0 | 0 | 0 | 0 | 0 |
| THYMU2017162 | 100 | 0 | 0 | 0 | 0 | 0 | 0 |
| THYMU2017215 | 100 | 0 | 0 | 0 | 0 | 0 | 0 |
| THYMU2017362 | 100 | 0 | 0 | 0 | 0 | 0 | 0 |
| THYMU2017366 | 100 | 0 | 0 | 0 | 0 | 0 | 0 |
| THYMU2017398 | 100 | 0 | 0 | 0 | 0 | 0 | 0 |
| THYMU2017449 | 100 | 0 | 0 | 0 | 0 | 0 | 0 |
| THYMU2017479 | 100 | 0 | 0 | 0 | 0 | 0 | 0 |
| THYMU2017522 | 100 | 0 | 0 | 0 | 0 | 0 | 0 |
| THYMU2017526 | 100 | 0 | 0 | 0 | 0 | 0 | 0 |
| THYMU2017601 | 5.889 | 0 | 0 | 0 | 0 | 7.910 | 0 |
| THYMU2017675 | 100 | 0 | 0 | 0 | 0 | 0 | 0 |
| THYMU2017707 | 100 | 0 | 0 | 0 | 0 | 0 | 0 |
| THYMU2017831 | 56.056 | 0 | 0 | 0 | 0 | 0 | 0 |
| THYMU2017844 | 100 | 0 | 0 | 0 | 0 | 0 | 0 |
| THYMU2017878 | 54.019 | 0 | 0 | 0 | 0 | 0 | 0 |
| THYMU2017948 | 71.841 | 0 | 0 | 0 | 0 | 0 | 0 |
| THYMU2018028 | 25.065 | 0 | 0 | 0 | 0 | 0 | 0 |
| THYMU2018071 | 100 | 0 | 0 | 0 | 0 | 0 | 0 |
| THYMU2018126 | 1.820 | 0 | 0 | 0 | 15.847 | 0 | 0 |
| THYMU2018189 | 100 | 0 | 0 | 0 | 0 | 0 | 0 |
| THYMU2018197 | 100 | 0 | 0 | 0 | 0 | 0 | 0 |
| THYMU2018384 | 100 | 0 | 0 | 0 | 0 | 0 | 0 |
| THYMU2018455 | 100 | 0 | 0 | 0 | 0 | 0 | 0 |
| THYMU2018547 | 100 | 0 | 0 | 0 | 0 | 0 | 0 |
| THYMU2018565 | 100 | 0 | 0 | 0 | 0 | 0 | 0 |
| THYMU2018639 | 32.401 | 0 | 0 | 0 | 0 | 0 | 0 |
| THYMU2018673 | 100 | 0 | 0 | 0 | 0 | 0 | 0 |
| THYMU2018721 | 100 | 0 | 0 | 0 | 0 | 0 | 0 |
| THYMU2018772 | 100 | 0 | 0 | 0 | 0 | 0 | 0 |
| THYMU2018819 | 100 | 0 | 0 | 0 | 0 | 0 | 0 |
| THYMU2018841 | 100 | 0 | 0 | 0 | 0 | 0 | 0 |
| THYMU2018997 | 100 | 0 | 0 | 0 | 0 | 0 | 0 |
| THYMU2019021 | 8.105 | 0 | 0 | 0 | 0 | 0 | 0 |
| THYMU2019054 | 100 | 0 | 0 | 0 | 0 | 0 | 0 |
| THYMU2019197 | 100 | 0 | 0 | 0 | 0 | 0 | 0 |
| THYMU2019210 | 100 | 0 | 0 | 0 | 0 | 0 | 0 |
| THYMU2019364 | 70.146 | 0 | 0 | 0 | 0 | 0 | 0 |
| THYMU2019436 | 100 | 0 | 0 | 0 | 0 | 0 | 0 |
| THYMU2019442 | 100 | 0 | 0 | 0 | 0 | 0 | 0 |
| THYMU2019587 | 100 | 0 | 0 | 0 | 0 | 0 | 0 |
| THYMU2019599 | 100 | 0 | 0 | 0 | 0 | 0 | 0 |
| THYMU2019686 | 100 | 0 | 0 | 0 | 0 | 0 | 0 |
| THYMU2019813 | 100 | 0 | 0 | 0 | 0 | 0 | 0 |
| THYMU2020198 | 100 | 0 | 0 | 0 | 0 | 0 | 0 |
| THYMU2020289 | 100 | 0 | 0 | 0 | 0 | 0 | 0 |
| THYMU2020416 | 11.429 | 0 | 0 | 0 | 0 | 0 | 0 |
| THYMU2020491 | 100 | 0 | 0 | 0 | 0 | 0 | 0 |
| THYMU2020499 | 100 | 0 | 0 | 0 | 0 | 0 | 0 |
| THYMU2020560 | 100 | 0 | 0 | 0 | 0 | 0 | 0 |
| THYMU2020667 | 58.303 | 0 | 0 | 0 | 0 | 0 | 0 |
| THYMU2020830 | 100 | 0 | 0 | 0 | 0 | 0 | 0 |
| THYMU2020883 | 42.920 | 0 | 0 | 0 | 0 | 0 | 0 |
| THYMU2020959 | 18.487 | 0 | 0 | 0 | 0 | 24.834 | 0 |
| THYMU2021361 | 100 | 0 | 0 | 0 | 0 | 0 | 0 |
| THYMU2021509 | 100 | 0 | 0 | 0 | 0 | 0 | 0 |
| THYMU2021597 | 100 | 0 | 0 | 0 | 0 | 0 | 0 |
| THYMU2021684 | 41.147 | 0 | 0 | 0 | 0 | 0 | 0 |
| THYMU2021714 | 100 | 0 | 0 | 0 | 0 | 0 | 0 |
| THYMU2022213 | 100 | 0 | 0 | 0 | 0 | 0 | 0 |
| THYMU2022289 | 100 | 0 | 0 | 0 | 0 | 0 | 0 |
| THYMU2022660 | 19.114 | 0 | 0 | 0 | 0 | 0 | 0 |
| THYMU2022854 | 100 | 0 | 0 | 0 | 0 | 0 | 0 |
| THYMU2022922 | 100 | 0 | 0 | 0 | 0 | 0 | 0 |
| THYMU2023209 | 100 | 0 | 0 | 0 | 0 | 0 | 0 |
| THYMU2023264 | 45.282 | 0 | 0 | 0 | 0 | 0 | 0 |
| THYMU2023576 | 26.584 | 0 | 0 | 0 | 0 | 0 | 0 |
| THYMU2023711 | 100 | 0 | 0 | 0 | 0 | 0 | 0 |
| THYMU2023900 | 100 | 0 | 0 | 0 | 0 | 0 | 0 |
| THYMU2023943 | 45.470 | 0 | 0 | 0 | 0 | 0 | 0 |
| THYMU2023967 | 25.472 | 0 | 0 | 0 | 0 | 0 | 0 |
| THYMU2024071 | 27.482 | 0 | 0 | 0 | 0 | 0 | 0 |
| THYMU2024121 | 48.944 | 0 | 0 | 0 | 0 | 0 | 0 |
| THYMU2024185 | 100 | 0 | 0 | 0 | 0 | 0 | 0 |
| THYMU2024207 | 100 | 0 | 0 | 0 | 0 | 0 | 0 |
| THYMU2024616 | 100 | 0 | 0 | 0 | 0 | 0 | 0 |
| THYMU2024684 | 100 | 0 | 0 | 0 | 0 | 0 | 0 |
| THYMU2024748 | 32.401 | 0 | 0 | 0 | 0 | 0 | 0 |
| THYMU2025042 | 100 | 0 | 0 | 0 | 0 | 0 | 0 |
| THYMU2025189 | 100 | 0 | 0 | 0 | 0 | 0 | 0 |
| THYMU2025319 | 27.551 | 0 | 0 | 0 | 0 | 0 | 0 |
| THYMU2025325 | 10.860 | 0 | 0 | 0 | 0 | 0 | 0 |
| THYMU2025406 | 100 | 0 | 0 | 0 | 0 | 0 | 0 |
| THYMU2025557 | 100 | 0 | 0 | 0 | 0 | 0 | 0 |
| THYMU2025572 | 100 | 0 | 0 | 0 | 0 | 0 | 0 |
| THYMU2025707 | 7.715 | 0 | 0 | 0 | 0 | 0 | 0 |
| THYMU2025849 | 100 | 0 | 0 | 0 | 0 | 0 | 0 |
| THYMU2025909 | 100 | 0 | 0 | 0 | 0 | 0 | 0 |
| THYMU2025939 | 26.596 | 0 | 0 | 0 | 0 | 53.592 | 0 |
| THYMU2026182 | 100 | 0 | 0 | 0 | 0 | 0 | 0 |
| THYMU2026350 | 40.164 | 0 | 0 | 0 | 0 | 0 | 0 |
| THYMU2026530 | 100 | 0 | 0 | 0 | 0 | 0 | 0 |
| THYMU2026531 | 100 | 0 | 0 | 0 | 0 | 0 | 0 |
| THYMU2026835 | 44.974 | 0 | 0 | 0 | 0 | 0 | 0 |
| THYMU2026904 | 100 | 0 | 0 | 0 | 0 | 0 | 0 |
| THYMU2027053 | 100 | 0 | 0 | 0 | 0 | 0 | 0 |
| THYMU2027125 | 0.299 | 0 | 0 | 0 | 0 | 0 | 0 |
| THYMU2027168 | 100 | 0 | 0 | 0 | 0 | 0 | 0 |
| THYMU2027249 | 100 | 0 | 0 | 0 | 0 | 0 | 0 |
| THYMU2027282 | 100 | 0 | 0 | 0 | 0 | 0 | 0 |
| THYMU2027497 | 100 | 0 | 0 | 0 | 0 | 0 | 0 |
| THYMU2027695 | 100 | 0 | 0 | 0 | 0 | 0 | 0 |
| THYMU2027734 | 100 | 0 | 0 | 0 | 0 | 0 | 0 |
| THYMU2027894 | 100 | 0 | 0 | 0 | 0 | 0 | 0 |
| THYMU2027975 | 3.333 | 0 | 0 | 0 | 0 | 0 | 0 |
| THYMU2027996 | 100 | 0 | 0 | 0 | 0 | 0 | 0 |
| THYMU2028041 | 100 | 0 | 0 | 0 | 0 | 0 | 0 |
| THYMU2028368 | 100 | 0 | 0 | 0 | 0 | 0 | 0 |
| THYMU2028379 | 100 | 0 | 0 | 0 | 0 | 0 | 0 |
| THYMU2028394 | 100 | 0 | 0 | 0 | 0 | 0 | 0 |
| THYMU2028412 | 100 | 0 | 0 | 0 | 0 | 0 | 0 |
| THYMU2028629 | 15.266 | 0 | 0 | 0 | 0 | 0 | 0 |
| THYMU2028632 | 100 | 0 | 0 | 0 | 0 | 0 | 0 |
| THYMU2028724 | 100 | 0 | 0 | 0 | 0 | 0 | 0 |
| THYMU2028739 | 56.056 | 0 | 0 | 0 | 0 | 0 | 0 |
| THYMU2028942 | 100 | 0 | 0 | 0 | 0 | 0 | 0 |
| THYMU2028978 | 100 | 0 | 0 | 0 | 0 | 0 | 0 |
| THYMU2029134 | 100 | 0 | 0 | 0 | 0 | 0 | 0 |
| THYMU2029578 | 22.934 | 0 | 0 | 0 | 0 | 0 | 0 |
| THYMU2029676 | 100 | 0 | 0 | 0 | 0 | 0 | 0 |
| THYMU2029688 | 100 | 0 | 0 | 0 | 0 | 0 | 0 |
| THYMU2029788 | 100 | 0 | 0 | 0 | 0 | 0 | 0 |
| THYMU2030068 | 100 | 0 | 0 | 0 | 0 | 0 | 0 |
| THYMU2030226 | 100 | 0 | 0 | 0 | 0 | 0 | 0 |
| THYMU2030264 | 54.019 | 0 | 0 | 0 | 0 | 0 | 0 |
| THYMU2030462 | 100 | 0 | 0 | 0 | 0 | 0 | 0 |
| THYMU2030543 | 100 | 0 | 0 | 0 | 0 | 0 | 0 |
| THYMU2030637 | 100 | 0 | 0 | 0 | 0 | 0 | 0 |
| THYMU2030655 | 18.385 | 81.615 | 0 | 0 | 0 | 0 | 0 |
| THYMU2030796 | 100 | 0 | 0 | 0 | 0 | 0 | 0 |
| THYMU2030912 | 100 | 0 | 0 | 0 | 0 | 0 | 0 |
| THYMU2030982 | 100 | 0 | 0 | 0 | 0 | 0 | 0 |
| THYMU2031046 | 100 | 0 | 0 | 0 | 0 | 0 | 0 |
| THYMU2031139 | 100 | 0 | 0 | 0 | 0 | 0 | 0 |
| THYMU2031218 | 100 | 0 | 0 | 0 | 0 | 0 | 0 |
| THYMU2031249 | 100 | 0 | 0 | 0 | 0 | 0 | 0 |
| THYMU2031258 | 100 | 0 | 0 | 0 | 0 | 0 | 0 |
| THYMU2031341 | 2.877 | 0 | 0 | 0 | 0 | 0 | 0 |
| THYMU2031368 | 3.784 | 0 | 0 | 0 | 0 | 0 | 0 |
| THYMU2031579 | 100 | 0 | 0 | 0 | 0 | 0 | 0 |
| THYMU2031847 | 30.924 | 0 | 0 | 0 | 0 | 0 | 0 |
| THYMU2031890 | 14.805 | 0 | 0 | 0 | 0 | 0 | 0 |
| THYMU2031994 | 100 | 0 | 0 | 0 | 0 | 0 | 0 |
| THYMU2032014 | 13.760 | 0 | 0 | 0 | 0 | 0 | 0 |
| THYMU2032035 | 100 | 0 | 0 | 0 | 0 | 0 | 0 |
| THYMU2032080 | 100 | 0 | 0 | 0 | 0 | 0 | 0 |
| THYMU2032150 | 100 | 0 | 0 | 0 | 0 | 0 | 0 |
| THYMU2032155 | 5.955 | 0 | 0 | 0 | 0 | 0 | 0 |
| THYMU2032358 | 100 | 0 | 0 | 0 | 0 | 0 | 0 |
| THYMU2032437 | 100 | 0 | 0 | 0 | 0 | 0 | 0 |
| THYMU2032601 | 100 | 0 | 0 | 0 | 0 | 0 | 0 |
| THYMU2032655 | 100 | 0 | 0 | 0 | 0 | 0 | 0 |
| THYMU2032696 | 0.876 | 0 | 0 | 0 | 0 | 0 | 0 |
| THYMU2032715 | 100 | 0 | 0 | 0 | 0 | 0 | 0 |
| THYMU2032732 | 100 | 0 | 0 | 0 | 0 | 0 | 0 |
| THYMU2032825 | 7.752 | 0 | 0 | 0 | 0 | 0 | 0 |
| THYMU2032976 | 100 | 0 | 0 | 0 | 0 | 0 | 0 |
| THYMU2033053 | 100 | 0 | 0 | 0 | 0 | 0 | 0 |
| THYMU2033059 | 18.523 | 0 | 0 | 0 | 0 | 0 | 0 |
| THYMU2033070 | 41.147 | 0 | 0 | 0 | 0 | 0 | 0 |
| THYMU2033079 | 100 | 0 | 0 | 0 | 0 | 0 | 0 |
| THYMU2033085 | 100 | 0 | 0 | 0 | 0 | 0 | 0 |
| THYMU2033104 | 100 | 0 | 0 | 0 | 0 | 0 | 0 |
| THYMU2033227 | 100 | 0 | 0 | 0 | 0 | 0 | 0 |
| THYMU2033263 | 9.276 | 0 | 0 | 0 | 0 | 0 | 0 |
| THYMU2033308 | 11.644 | 0 | 0 | 0 | 0 | 0 | 0 |
| THYMU2033401 | 100 | 0 | 0 | 0 | 0 | 0 | 0 |
| THYMU2033583 | 42.674 | 0 | 0 | 0 | 0 | 57.326 | 0 |
| THYMU2033755 | 100 | 0 | 0 | 0 | 0 | 0 | 0 |
| THYMU2033787 | 42.920 | 0 | 0 | 0 | 0 | 0 | 0 |
| THYMU2033816 | 100 | 0 | 0 | 0 | 0 | 0 | 0 |
| THYMU2034182 | 100 | 0 | 0 | 0 | 0 | 0 | 0 |
| THYMU2034279 | 100 | 0 | 0 | 0 | 0 | 0 | 0 |
| THYMU2034314 | 100 | 0 | 0 | 0 | 0 | 0 | 0 |
| THYMU2034338 | 100 | 0 | 0 | 0 | 0 | 0 | 0 |
| THYMU2034374 | 10.489 | 0 | 0 | 0 | 0 | 0 | 0 |
| THYMU2034647 | 32.401 | 0 | 0 | 0 | 0 | 0 | 0 |
| THYMU2034917 | 16.203 | 0 | 0 | 0 | 0 | 0 | 0 |
| THYMU2035064 | 100 | 0 | 0 | 0 | 0 | 0 | 0 |
| THYMU2035078 | 100 | 0 | 0 | 0 | 0 | 0 | 0 |
| THYMU2035101 | 100 | 0 | 0 | 0 | 0 | 0 | 0 |
| THYMU2035319 | 100 | 0 | 0 | 0 | 0 | 0 | 0 |
| THYMU2035321 | 100 | 0 | 0 | 0 | 0 | 0 | 0 |
| THYMU2035388 | 100 | 0 | 0 | 0 | 0 | 0 | 0 |
| THYMU2035400 | 100 | 0 | 0 | 0 | 0 | 0 | 0 |
| THYMU2035554 | 100 | 0 | 0 | 0 | 0 | 0 | 0 |
| THYMU2035710 | 100 | 0 | 0 | 0 | 0 | 0 | 0 |
| THYMU2035735 | 2.200 | 0 | 0 | 0 | 0 | 5.911 | 28.332 |
| THYMU2036058 | 56.056 | 0 | 0 | 0 | 0 | 0 | 0 |
| THYMU2036105 | 100 | 0 | 0 | 0 | 0 | 0 | 0 |
| THYMU2036207 | 100 | 0 | 0 | 0 | 0 | 0 | 0 |
| THYMU2036215 | 19.182 | 0 | 0 | 0 | 0 | 0 | 0 |
| THYMU2036252 | 100 | 0 | 0 | 0 | 0 | 0 | 0 |
| THYMU2036265 | 100 | 0 | 0 | 0 | 0 | 0 | 0 |
| THYMU2036366 | 100 | 0 | 0 | 0 | 0 | 0 | 0 |
| THYMU2036459 | 100 | 0 | 0 | 0 | 0 | 0 | 0 |
| THYMU2036461 | 12.647 | 0 | 0 | 0 | 0 | 0 | 0 |
| THYMU2036604 | 100 | 0 | 0 | 0 | 0 | 0 | 0 |
| THYMU2036653 | 100 | 0 | 0 | 0 | 0 | 0 | 0 |
| THYMU2037081 | 100 | 0 | 0 | 0 | 0 | 0 | 0 |
| THYMU2037208 | 100 | 0 | 0 | 0 | 0 | 0 | 0 |
| THYMU2037226 | 28.577 | 0 | 0 | 0 | 0 | 19.194 | 0 |
| THYMU2037233 | 100 | 0 | 0 | 0 | 0 | 0 | 0 |
| THYMU2037234 | 100 | 0 | 0 | 0 | 0 | 0 | 0 |
| THYMU2037348 | 100 | 0 | 0 | 0 | 0 | 0 | 0 |
| THYMU2037406 | 100 | 0 | 0 | 0 | 0 | 0 | 0 |
| THYMU2037965 | 100 | 0 | 0 | 0 | 0 | 0 | 0 |
| THYMU2038058 | 100 | 0 | 0 | 0 | 0 | 0 | 0 |
| THYMU2038189 | 100 | 0 | 0 | 0 | 0 | 0 | 0 |
| THYMU2038199 | 100 | 0 | 0 | 0 | 0 | 0 | 0 |
| THYMU2038220 | 16.844 | 0 | 0 | 0 | 0 | 0 | 0 |
| THYMU2038301 | 100 | 0 | 0 | 0 | 0 | 0 | 0 |
| THYMU2038369 | 100 | 0 | 0 | 0 | 0 | 0 | 0 |
| THYMU2038389 | 100 | 0 | 0 | 0 | 0 | 0 | 0 |
| THYMU2038615 | 100 | 0 | 0 | 0 | 0 | 0 | 0 |
| THYMU2038636 | 100 | 0 | 0 | 0 | 0 | 0 | 0 |
| THYMU2038739 | 100 | 0 | 0 | 0 | 0 | 0 | 0 |
| THYMU2038772 | 100 | 0 | 0 | 0 | 0 | 0 | 0 |
| THYMU2038797 | 100 | 0 | 0 | 0 | 0 | 0 | 0 |
| THYMU2038906 | 100 | 0 | 0 | 0 | 0 | 0 | 0 |
| THYMU2039305 | 100 | 0 | 0 | 0 | 0 | 0 | 0 |
| THYMU2039315 | 45.470 | 0 | 0 | 0 | 0 | 0 | 0 |
| THYMU2039334 | 0.947 | 0 | 0 | 0 | 0 | 0 | 0 |
| THYMU2039350 | 56.056 | 0 | 0 | 0 | 0 | 0 | 0 |
| THYMU2039411 | 100 | 0 | 0 | 0 | 0 | 0 | 0 |
| THYMU2039634 | 45.470 | 0 | 0 | 0 | 0 | 0 | 0 |
| THYMU2039719 | 100 | 0 | 0 | 0 | 0 | 0 | 0 |
| THYMU2039780 | 54.019 | 0 | 0 | 0 | 0 | 0 | 0 |
| THYMU2039788 | 100 | 0 | 0 | 0 | 0 | 0 | 0 |
| THYMU2039989 | 100 | 0 | 0 | 0 | 0 | 0 | 0 |
| THYMU2040029 | 100 | 0 | 0 | 0 | 0 | 0 | 0 |
| THYMU2040110 | 100 | 0 | 0 | 0 | 0 | 0 | 0 |
| THYMU2040114 | 26.657 | 0 | 0 | 0 | 0 | 0 | 0 |
| THYMU2040140 | 100 | 0 | 0 | 0 | 0 | 0 | 0 |
| THYMU2040412 | 100 | 0 | 0 | 0 | 0 | 0 | 0 |
| THYMU2040427 | 54.019 | 0 | 0 | 0 | 0 | 0 | 0 |
| THYMU2040824 | 100 | 0 | 0 | 0 | 0 | 0 | 0 |
| THYMU2040925 | 54.019 | 0 | 0 | 0 | 0 | 0 | 0 |
| THYMU2041007 | 100 | 0 | 0 | 0 | 0 | 0 | 0 |
| THYMU2041015 | 100 | 0 | 0 | 0 | 0 | 0 | 0 |
| THYMU2041252 | 100 | 0 | 0 | 0 | 0 | 0 | 0 |
| THYMU3000028 | 100 | 0 | 0 | 0 | 0 | 0 | 0 |
| THYMU3000036 | 100 | 0 | 0 | 0 | 0 | 0 | 0 |
| THYMU3000037 | 100 | 0 | 0 | 0 | 0 | 0 | 0 |
| THYMU3000082 | 100 | 0 | 0 | 0 | 0 | 0 | 0 |
| THYMU3000194 | 100 | 0 | 0 | 0 | 0 | 0 | 0 |
| THYMU3000224 | 100 | 0 | 0 | 0 | 0 | 0 | 0 |
| THYMU3000269 | 100 | 0 | 0 | 0 | 0 | 0 | 0 |
| THYMU3000306 | 100 | 0 | 0 | 0 | 0 | 0 | 0 |
| THYMU3000360 | 32.782 | 0 | 0 | 0 | 0 | 0 | 0 |
| THYMU3000390 | 100 | 0 | 0 | 0 | 0 | 0 | 0 |
| THYMU3000420 | 32.782 | 0 | 0 | 0 | 0 | 0 | 0 |
| THYMU3000490 | 100 | 0 | 0 | 0 | 0 | 0 | 0 |
| THYMU3000504 | 100 | 0 | 0 | 0 | 0 | 0 | 0 |
| THYMU3000542 | 100 | 0 | 0 | 0 | 0 | 0 | 0 |
| THYMU3000655 | 42.674 | 0 | 0 | 0 | 0 | 57.326 | 0 |
| THYMU3000776 | 52.046 | 0 | 0 | 0 | 0 | 0 | 0 |
| THYMU3000826 | 100 | 0 | 0 | 0 | 0 | 0 | 0 |
| THYMU3000841 | 100 | 0 | 0 | 0 | 0 | 0 | 0 |
| THYMU3001077 | 25.065 | 0 | 0 | 0 | 0 | 0 | 0 |
| THYMU3001082 | 100 | 0 | 0 | 0 | 0 | 0 | 0 |
| THYMU3001083 | 54.019 | 0 | 0 | 0 | 0 | 0 | 0 |
| THYMU3001133 | 100 | 0 | 0 | 0 | 0 | 0 | 0 |
| THYMU3001151 | 100 | 0 | 0 | 0 | 0 | 0 | 0 |
| THYMU3001379 | 21.000 | 0 | 0 | 0 | 0 | 0 | 0 |
| THYMU3001472 | 42.153 | 0 | 0 | 0 | 0 | 37.750 | 0 |
| THYMU3001593 | 100 | 0 | 0 | 0 | 0 | 0 | 0 |
| THYMU3001883 | 100 | 0 | 0 | 0 | 0 | 0 | 0 |
| THYMU3001939 | 40.164 | 0 | 0 | 0 | 0 | 0 | 0 |
| THYMU3001991 | 100 | 0 | 0 | 0 | 0 | 0 | 0 |
| THYMU3002452 | 100 | 0 | 0 | 0 | 0 | 0 | 0 |
| THYMU3002578 | 25.509 | 0 | 0 | 0 | 0 | 0 | 0 |
| THYMU3002661 | 100 | 0 | 0 | 0 | 0 | 0 | 0 |
| THYMU3003007 | 47.545 | 0 | 0 | 0 | 0 | 0 | 0 |
| THYMU3003092 | 100 | 0 | 0 | 0 | 0 | 0 | 0 |
| THYMU3003212 | 100 | 0 | 0 | 0 | 0 | 0 | 0 |
| THYMU3003309 | 24.243 | 0 | 0 | 0 | 0 | 0 | 0 |
| THYMU3003350 | 45.470 | 0 | 0 | 0 | 0 | 0 | 0 |
| THYMU3003403 | 100 | 0 | 0 | 0 | 0 | 0 | 0 |
| THYMU3003565 | 48.944 | 0 | 0 | 0 | 0 | 0 | 0 |
| THYMU3003763 | 100 | 0 | 0 | 0 | 0 | 0 | 0 |
| THYMU3003865 | 17.533 | 0 | 0 | 0 | 0 | 0 | 0 |
| THYMU3003958 | 100 | 0 | 0 | 0 | 0 | 0 | 0 |
| THYMU3003965 | 44.974 | 0 | 0 | 0 | 0 | 0 | 0 |
| THYMU3004157 | 100 | 0 | 0 | 0 | 0 | 0 | 0 |
| THYMU3004628 | 100 | 0 | 0 | 0 | 0 | 0 | 0 |
| THYMU3004632 | 100 | 0 | 0 | 0 | 0 | 0 | 0 |
| THYMU3004835 | 16.380 | 0 | 0 | 0 | 0 | 22.003 | 0 |
| THYMU3004866 | 100 | 0 | 0 | 0 | 0 | 0 | 0 |
| THYMU3005050 | 100 | 0 | 0 | 0 | 0 | 0 | 0 |
| THYMU3005378 | 100 | 0 | 0 | 0 | 0 | 0 | 0 |
| THYMU3005407 | 100 | 0 | 0 | 0 | 0 | 0 | 0 |
| THYMU3005415 | 100 | 0 | 0 | 0 | 0 | 0 | 0 |
| THYMU3005427 | 19.680 | 0 | 0 | 0 | 0 | 0 | 0 |
| THYMU3005482 | 100 | 0 | 0 | 0 | 0 | 0 | 0 |
| THYMU3005491 | 42.920 | 0 | 0 | 0 | 0 | 0 | 0 |
| THYMU3005629 | 12.599 | 0 | 0 | 0 | 0 | 0 | 0 |
| THYMU3005696 | 100 | 0 | 0 | 0 | 0 | 0 | 0 |
| THYMU3006118 | 100 | 0 | 0 | 0 | 0 | 0 | 0 |
| THYMU3006132 | 100 | 0 | 0 | 0 | 0 | 0 | 0 |
| THYMU3006168 | 100 | 0 | 0 | 0 | 0 | 0 | 0 |
| THYMU3006172 | 42.674 | 0 | 0 | 0 | 0 | 57.326 | 0 |
| THYMU3006371 | 100 | 0 | 0 | 0 | 0 | 0 | 0 |
| THYMU3006485 | 54.019 | 0 | 0 | 0 | 0 | 0 | 0 |
| THYMU3006640 | 100 | 0 | 0 | 0 | 0 | 0 | 0 |
| THYMU3006767 | 42.674 | 0 | 0 | 0 | 0 | 57.326 | 0 |
| THYMU3006811 | 42.674 | 0 | 0 | 0 | 0 | 57.326 | 0 |
| THYMU3006963 | 100 | 0 | 0 | 0 | 0 | 0 | 0 |
| THYMU3007137 | 62.044 | 0 | 0 | 0 | 0 | 0 | 0 |
| THYMU3007308 | 32.523 | 0 | 0 | 0 | 0 | 0 | 0 |
| THYMU3007368 | 100 | 0 | 0 | 0 | 0 | 0 | 0 |
| THYMU3007423 | 24.596 | 0 | 0 | 0 | 0 | 11.014 | 0 |
| THYMU3007559 | 100 | 0 | 0 | 0 | 0 | 0 | 0 |
| THYMU3007845 | 100 | 0 | 0 | 0 | 0 | 0 | 0 |
| THYMU3008060 | 100 | 0 | 0 | 0 | 0 | 0 | 0 |
| THYMU3008105 | 33.774 | 0 | 0 | 0 | 0 | 0 | 0 |
| THYMU3008136 | 100 | 0 | 0 | 0 | 0 | 0 | 0 |
| THYMU3008171 | 33.638 | 0 | 0 | 0 | 0 | 11.297 | 0 |
| THYMU3008216 | 100 | 0 | 0 | 0 | 0 | 0 | 0 |
| THYMU3008436 | 100 | 0 | 0 | 0 | 0 | 0 | 0 |
| THYMU3008672 | 100 | 0 | 0 | 0 | 0 | 0 | 0 |
| THYMU3008883 | 20.284 | 0 | 0 | 0 | 0 | 27.248 | 0 |
| THYMU3008935 | 100 | 0 | 0 | 0 | 0 | 0 | 0 |
| THYMU3009006 | 46.409 | 0 | 0 | 0 | 0 | 0 | 0 |
| THYMU3009255 | 100 | 0 | 0 | 0 | 0 | 0 | 0 |
| THYMU3009391 | 100 | 0 | 0 | 0 | 0 | 0 | 0 |
| THYMU3009643 | 20.327 | 0 | 0 | 0 | 0 | 0 | 0 |
| THYMU3009755 | 100 | 0 | 0 | 0 | 0 | 0 | 0 |
| THYMU3010247 | 100 | 0 | 0 | 0 | 0 | 0 | 0 |
| THYMU3011012 | 100 | 0 | 0 | 0 | 0 | 0 | 0 |
| THYMU3011081 | 100 | 0 | 0 | 0 | 0 | 0 | 0 |
| THYMU3011244 | 100 | 0 | 0 | 0 | 0 | 0 | 0 |
| THYMU3011360 | 100 | 0 | 0 | 0 | 0 | 0 | 0 |
| THYMU3011534 | 100 | 0 | 0 | 0 | 0 | 0 | 0 |
| THYMU3011543 | 100 | 0 | 0 | 0 | 0 | 0 | 0 |
| THYMU3011556 | 100 | 0 | 0 | 0 | 0 | 0 | 0 |
| THYMU3011717 | 100 | 0 | 0 | 0 | 0 | 0 | 0 |
| THYMU3011827 | 19.368 | 0 | 0 | 0 | 0 | 26.018 | 0 |
| THYMU3012402 | 100 | 0 | 0 | 0 | 0 | 0 | 0 |
| THYMU3012907 | 100 | 0 | 0 | 0 | 0 | 0 | 0 |
| THYMU3012983 | 56.056 | 0 | 0 | 0 | 0 | 0 | 0 |
| THYMU3013114 | 100 | 0 | 0 | 0 | 0 | 0 | 0 |
| THYMU3013197 | 100 | 0 | 0 | 0 | 0 | 0 | 0 |
| THYMU3013241 | 100 | 0 | 0 | 0 | 0 | 0 | 0 |
| THYMU3013386 | 3.121 | 0 | 0 | 0 | 0 | 0 | 0 |
| THYMU3013470 | 100 | 0 | 0 | 0 | 0 | 0 | 0 |
| THYMU3013785 | 100 | 0 | 0 | 0 | 0 | 0 | 0 |
| THYMU3013897 | 100 | 0 | 0 | 0 | 0 | 0 | 0 |
| THYMU3014038 | 100 | 0 | 0 | 0 | 0 | 0 | 0 |
| THYMU3014173 | 100 | 0 | 0 | 0 | 0 | 0 | 0 |
| THYMU3014372 | 100 | 0 | 0 | 0 | 0 | 0 | 0 |
| THYMU3014555 | 100 | 0 | 0 | 0 | 0 | 0 | 0 |
| THYMU3014620 | 100 | 0 | 0 | 0 | 0 | 0 | 0 |
| THYMU3014701 | 100 | 0 | 0 | 0 | 0 | 0 | 0 |
| THYMU3015042 | 100 | 0 | 0 | 0 | 0 | 0 | 0 |
| THYMU3015457 | 100 | 0 | 0 | 0 | 0 | 0 | 0 |
| THYMU3015571 | 26.275 | 0 | 0 | 0 | 0 | 0 | 0 |
| THYMU3015592 | 100 | 0 | 0 | 0 | 0 | 0 | 0 |
| THYMU3015625 | 100 | 0 | 0 | 0 | 0 | 0 | 0 |
| THYMU3015647 | 100 | 0 | 0 | 0 | 0 | 0 | 0 |
| THYMU3015759 | 8.449 | 0 | 0 | 23.299 | 0 | 1.261 | 0 |
| THYMU3015962 | 100 | 0 | 0 | 0 | 0 | 0 | 0 |
| THYMU3016022 | 100 | 0 | 0 | 0 | 0 | 0 | 0 |
| THYMU3016518 | 100 | 0 | 0 | 0 | 0 | 0 | 0 |
| THYMU3016797 | 100 | 0 | 0 | 0 | 0 | 0 | 0 |
| THYMU3016822 | 100 | 0 | 0 | 0 | 0 | 0 | 0 |
| THYMU3016850 | 100 | 0 | 0 | 0 | 0 | 0 | 0 |
| THYMU3017562 | 100 | 0 | 0 | 0 | 0 | 0 | 0 |
| THYMU3017688 | 100 | 0 | 0 | 0 | 0 | 0 | 0 |
| THYMU3017689 | 100 | 0 | 0 | 0 | 0 | 0 | 0 |
| THYMU3017761 | 100 | 0 | 0 | 0 | 0 | 0 | 0 |
| THYMU3018151 | 100 | 0 | 0 | 0 | 0 | 0 | 0 |
| THYMU3018896 | 100 | 0 | 0 | 0 | 0 | 0 | 0 |
| THYMU3019095 | 100 | 0 | 0 | 0 | 0 | 0 | 0 |
| THYMU3019476 | 45.282 | 0 | 0 | 0 | 0 | 0 | 0 |
| THYMU3019605 | 100 | 0 | 0 | 0 | 0 | 0 | 0 |
| THYMU3019916 | 100 | 0 | 0 | 0 | 0 | 0 | 0 |
| THYMU3020221 | 100 | 0 | 0 | 0 | 0 | 0 | 0 |
| THYMU3020595 | 100 | 0 | 0 | 0 | 0 | 0 | 0 |
| THYMU3020713 | 100 | 0 | 0 | 0 | 0 | 0 | 0 |
| THYMU3020856 | 100 | 0 | 0 | 0 | 0 | 0 | 0 |
| THYMU3020869 | 100 | 0 | 0 | 0 | 0 | 0 | 0 |
| THYMU3020970 | 100 | 0 | 0 | 0 | 0 | 0 | 0 |
| THYMU3021404 | 100 | 0 | 0 | 0 | 0 | 0 | 0 |
| THYMU3021586 | 13.776 | 0 | 0 | 0 | 0 | 4.626 | 0 |
| THYMU3021755 | 100 | 0 | 0 | 0 | 0 | 0 | 0 |
| THYMU3021900 | 100 | 0 | 0 | 0 | 0 | 0 | 0 |
| THYMU3022211 | 100 | 0 | 0 | 0 | 0 | 0 | 0 |
| THYMU3022434 | 100 | 0 | 0 | 0 | 0 | 0 | 0 |
| THYMU3022528 | 100 | 0 | 0 | 0 | 0 | 0 | 0 |
| THYMU3022668 | 100 | 0 | 0 | 0 | 0 | 0 | 0 |
| THYMU3022982 | 100 | 0 | 0 | 0 | 0 | 0 | 0 |
| THYMU3023107 | 100 | 0 | 0 | 0 | 0 | 0 | 0 |
| THYMU3023211 | 100 | 0 | 0 | 0 | 0 | 0 | 0 |
| THYMU3023216 | 100 | 0 | 0 | 0 | 0 | 0 | 0 |
| THYMU3023394 | 100 | 0 | 0 | 0 | 0 | 0 | 0 |
| THYMU3023400 | 100 | 0 | 0 | 0 | 0 | 0 | 0 |
| THYMU3023797 | 100 | 0 | 0 | 0 | 0 | 0 | 0 |
| THYMU3024164 | 100 | 0 | 0 | 0 | 0 | 0 | 0 |
| THYMU3024339 | 48.376 | 0 | 0 | 0 | 0 | 0 | 0 |
| THYMU3024602 | 100 | 0 | 0 | 0 | 0 | 0 | 0 |
| THYMU3024879 | 100 | 0 | 0 | 0 | 0 | 0 | 0 |
| THYMU3025118 | 100 | 0 | 0 | 0 | 0 | 0 | 0 |
| THYMU3025313 | 100 | 0 | 0 | 0 | 0 | 0 | 0 |
| THYMU3025642 | 31.905 | 0 | 0 | 0 | 0 | 0 | 0 |
| THYMU3025683 | 100 | 0 | 0 | 0 | 0 | 0 | 0 |
| THYMU3025772 | 100 | 0 | 0 | 0 | 0 | 0 | 0 |
| THYMU3025865 | 100 | 0 | 0 | 0 | 0 | 0 | 0 |
| THYMU3026000 | 20.457 | 0 | 0 | 0 | 0 | 0 | 0 |
| THYMU3026350 | 23.834 | 0 | 0 | 0 | 0 | 0 | 0 |
| THYMU3026479 | 100 | 0 | 0 | 0 | 0 | 0 | 0 |
| THYMU3026532 | 100 | 0 | 0 | 0 | 0 | 0 | 0 |
| THYMU3026783 | 100 | 0 | 0 | 0 | 0 | 0 | 0 |
| THYMU3026869 | 100 | 0 | 0 | 0 | 0 | 0 | 0 |
| THYMU3027251 | 100 | 0 | 0 | 0 | 0 | 0 | 0 |
| THYMU3027540 | 100 | 0 | 0 | 0 | 0 | 0 | 0 |
| THYMU3027655 | 100 | 0 | 0 | 0 | 0 | 0 | 0 |
| THYMU3027671 | 100 | 0 | 0 | 0 | 0 | 0 | 0 |
| THYMU3028075 | 100 | 0 | 0 | 0 | 0 | 0 | 0 |
| THYMU3028410 | 41.147 | 0 | 0 | 0 | 0 | 0 | 0 |
| THYMU3028461 | 100 | 0 | 0 | 0 | 0 | 0 | 0 |
| THYMU3028702 | 100 | 0 | 0 | 0 | 0 | 0 | 0 |
| THYMU3029188 | 100 | 0 | 0 | 0 | 0 | 0 | 0 |
| THYMU3029318 | 100 | 0 | 0 | 0 | 0 | 0 | 0 |
| THYMU3029421 | 100 | 0 | 0 | 0 | 0 | 0 | 0 |
| THYMU3029719 | 100 | 0 | 0 | 0 | 0 | 0 | 0 |
| THYMU3029774 | 100 | 0 | 0 | 0 | 0 | 0 | 0 |
| THYMU3029832 | 100 | 0 | 0 | 0 | 0 | 0 | 0 |
| THYMU3030072 | 100 | 0 | 0 | 0 | 0 | 0 | 0 |
| THYMU3030231 | 100 | 0 | 0 | 0 | 0 | 0 | 0 |
| THYMU3030706 | 100 | 0 | 0 | 0 | 0 | 0 | 0 |
| THYMU3030710 | 100 | 0 | 0 | 0 | 0 | 0 | 0 |
| THYMU3030752 | 100 | 0 | 0 | 0 | 0 | 0 | 0 |
| THYMU3031146 | 100 | 0 | 0 | 0 | 0 | 0 | 0 |
| THYMU3031402 | 100 | 0 | 0 | 0 | 0 | 0 | 0 |
| THYMU3031610 | 100 | 0 | 0 | 0 | 0 | 0 | 0 |
| THYMU3031612 | 100 | 0 | 0 | 0 | 0 | 0 | 0 |
| THYMU3031868 | 100 | 0 | 0 | 0 | 0 | 0 | 0 |
| THYMU3031878 | 100 | 0 | 0 | 0 | 0 | 0 | 0 |
| THYMU3032032 | 100 | 0 | 0 | 0 | 0 | 0 | 0 |
| THYMU3032798 | 17.736 | 0 | 0 | 0 | 0 | 2.166 | 0 |
| THYMU3032867 | 10.732 | 0 | 0 | 0 | 0 | 0 | 0 |
| THYMU3033402 | 100 | 0 | 0 | 0 | 0 | 0 | 0 |
| THYMU3033626 | 9.374 | 0 | 0 | 0 | 0 | 0 | 0 |
| THYMU3033630 | 100 | 0 | 0 | 0 | 0 | 0 | 0 |
| THYMU3033649 | 100 | 0 | 0 | 0 | 0 | 0 | 0 |
| THYMU3033754 | 100 | 0 | 0 | 0 | 0 | 0 | 0 |
| THYMU3033759 | 100 | 0 | 0 | 0 | 0 | 0 | 0 |
| THYMU3034078 | 100 | 0 | 0 | 0 | 0 | 0 | 0 |
| THYMU3034099 | 100 | 0 | 0 | 0 | 0 | 0 | 0 |
| THYMU3034453 | 100 | 0 | 0 | 0 | 0 | 0 | 0 |
| THYMU3034616 | 41.147 | 0 | 0 | 0 | 0 | 0 | 0 |
| THYMU3034671 | 9.943 | 0 | 0 | 0 | 0 | 5.343 | 0 |
| THYMU3034853 | 100 | 0 | 0 | 0 | 0 | 0 | 0 |
| THYMU3034867 | 100 | 0 | 0 | 0 | 0 | 0 | 0 |
| THYMU3034983 | 100 | 0 | 0 | 0 | 0 | 0 | 0 |
| THYMU3036200 | 100 | 0 | 0 | 0 | 0 | 0 | 0 |
| THYMU3036310 | 100 | 0 | 0 | 0 | 0 | 0 | 0 |
| THYMU3036934 | 100 | 0 | 0 | 0 | 0 | 0 | 0 |
| THYMU3036953 | 100 | 0 | 0 | 0 | 0 | 0 | 0 |
| THYMU3037052 | 100 | 0 | 0 | 0 | 0 | 0 | 0 |
| THYMU3037192 | 100 | 0 | 0 | 0 | 0 | 0 | 0 |
| THYMU3037617 | 100 | 0 | 0 | 0 | 0 | 0 | 0 |
| THYMU3037772 | 100 | 0 | 0 | 0 | 0 | 0 | 0 |
| THYMU3037827 | 45.470 | 0 | 0 | 0 | 0 | 0 | 0 |
| THYMU3037836 | 100 | 0 | 0 | 0 | 0 | 0 | 0 |
| THYMU3037856 | 100 | 0 | 0 | 0 | 0 | 0 | 0 |
| THYMU3037867 | 100 | 0 | 0 | 0 | 0 | 0 | 0 |
| THYMU3037909 | 100 | 0 | 0 | 0 | 0 | 0 | 0 |
| THYMU3037980 | 100 | 0 | 0 | 0 | 0 | 0 | 0 |
| THYMU3038158 | 5.135 | 0 | 0 | 0 | 0 | 0 | 0 |
| THYMU3038167 | 100 | 0 | 0 | 0 | 0 | 0 | 0 |
| THYMU3038214 | 32.782 | 0 | 0 | 0 | 0 | 0 | 0 |
| THYMU3038266 | 100 | 0 | 0 | 0 | 0 | 0 | 0 |
| THYMU3038328 | 100 | 0 | 0 | 0 | 0 | 0 | 0 |
| THYMU3038347 | 100 | 0 | 0 | 0 | 0 | 0 | 0 |
| THYMU3038375 | 100 | 0 | 0 | 0 | 0 | 0 | 0 |
| THYMU3038603 | 100 | 0 | 0 | 0 | 0 | 0 | 0 |
| THYMU3038687 | 100 | 0 | 0 | 0 | 0 | 0 | 0 |
| THYMU3038759 | 100 | 0 | 0 | 0 | 0 | 0 | 0 |
| THYMU3038879 | 100 | 0 | 0 | 0 | 0 | 0 | 0 |
| THYMU3038970 | 100 | 0 | 0 | 0 | 0 | 0 | 0 |
| THYMU3039807 | 100 | 0 | 0 | 0 | 0 | 0 | 0 |
| THYMU3039846 | 100 | 0 | 0 | 0 | 0 | 0 | 0 |
| THYMU3040068 | 32.401 | 0 | 0 | 0 | 0 | 0 | 0 |
| THYMU3040126 | 100 | 0 | 0 | 0 | 0 | 0 | 0 |
| THYMU3040146 | 100 | 0 | 0 | 0 | 0 | 0 | 0 |
| THYMU3040168 | 100 | 0 | 0 | 0 | 0 | 0 | 0 |
| THYMU3040172 | 100 | 0 | 0 | 0 | 0 | 0 | 0 |
| THYMU3040725 | 100 | 0 | 0 | 0 | 0 | 0 | 0 |
| THYMU3040746 | 100 | 0 | 0 | 0 | 0 | 0 | 0 |
| THYMU3040816 | 9.663 | 0 | 0 | 0 | 0 | 0 | 0 |
| THYMU3040829 | 100 | 0 | 0 | 0 | 0 | 0 | 0 |
| THYMU3040830 | 100 | 0 | 0 | 0 | 0 | 0 | 0 |
| THYMU3040986 | 100 | 0 | 0 | 0 | 0 | 0 | 0 |
| THYMU3041354 | 100 | 0 | 0 | 0 | 0 | 0 | 0 |
| THYMU3041386 | 100 | 0 | 0 | 0 | 0 | 0 | 0 |
| THYMU3041428 | 0.213 | 0 | 0 | 0 | 0 | 0 | 0 |
| THYMU3041573 | 100 | 0 | 0 | 0 | 0 | 0 | 0 |
| THYMU3041603 | 100 | 0 | 0 | 0 | 0 | 0 | 0 |
| THYMU3041736 | 100 | 0 | 0 | 0 | 0 | 0 | 0 |
| THYMU3041918 | 100 | 0 | 0 | 0 | 0 | 0 | 0 |
| THYMU3042075 | 100 | 0 | 0 | 0 | 0 | 0 | 0 |
| THYMU3042321 | 100 | 0 | 0 | 0 | 0 | 0 | 0 |
| THYMU3042372 | 100 | 0 | 0 | 0 | 0 | 0 | 0 |
| THYMU3042758 | 100 | 0 | 0 | 0 | 0 | 0 | 0 |
| THYMU3042929 | 100 | 0 | 0 | 0 | 0 | 0 | 0 |
| THYMU3043200 | 100 | 0 | 0 | 0 | 0 | 0 | 0 |
| THYMU3043327 | 100 | 0 | 0 | 0 | 0 | 0 | 0 |
| THYMU3043482 | 100 | 0 | 0 | 0 | 0 | 0 | 0 |
| THYMU3043597 | 100 | 0 | 0 | 0 | 0 | 0 | 0 |
| THYMU3043688 | 100 | 0 | 0 | 0 | 0 | 0 | 0 |
| THYMU3043779 | 100 | 0 | 0 | 0 | 0 | 0 | 0 |
| THYMU3043883 | 100 | 0 | 0 | 0 | 0 | 0 | 0 |
| THYMU3043993 | 100 | 0 | 0 | 0 | 0 | 0 | 0 |
| THYMU3044175 | 100 | 0 | 0 | 0 | 0 | 0 | 0 |
| THYMU3044188 | 3.732 | 0 | 0 | 0 | 0 | 0 | 0 |
| THYMU3044441 | 100 | 0 | 0 | 0 | 0 | 0 | 0 |
| THYMU3044445 | 100 | 0 | 0 | 0 | 0 | 0 | 0 |
| THYMU3045510 | 58.982 | 0 | 0 | 0 | 0 | 0 | 0 |
| THYMU3045673 | 100 | 0 | 0 | 0 | 0 | 0 | 0 |
| THYMU3045692 | 100 | 0 | 0 | 0 | 0 | 0 | 0 |
| THYMU3045704 | 100 | 0 | 0 | 0 | 0 | 0 | 0 |
| THYMU3046140 | 100 | 0 | 0 | 0 | 0 | 0 | 0 |
| THYMU3046350 | 5.760 | 0 | 47.489 | 0 | 0 | 0 | 0 |
| THYMU3046360 | 100 | 0 | 0 | 0 | 0 | 0 | 0 |
| THYMU3046856 | 100 | 0 | 0 | 0 | 0 | 0 | 0 |
| THYMU3047115 | 0.343 | 0 | 0 | 0 | 0 | 0 | 0 |
| THYMU3047144 | 100 | 0 | 0 | 0 | 0 | 0 | 0 |
| THYMU3047156 | 100 | 0 | 0 | 0 | 0 | 0 | 0 |
| THYMU3047366 | 100 | 0 | 0 | 0 | 0 | 0 | 0 |
| THYMU3047513 | 100 | 0 | 0 | 0 | 0 | 0 | 0 |
| THYMU3047542 | 100 | 0 | 0 | 0 | 0 | 0 | 0 |
| THYMU3047760 | 100 | 0 | 0 | 0 | 0 | 0 | 0 |
| THYMU3047891 | 100 | 0 | 0 | 0 | 0 | 0 | 0 |
| TKIDN1000001 | 0 | 41.198 | 0 | 0 | 0 | 0 | 0 |
| TKIDN1000044 | 0 | 34.663 | 0 | 0 | 0 | 0 | 0 |
| TKIDN1000062 | 0 | 100 | 0 | 0 | 0 | 0 | 0 |
| TKIDN1000066 | 0 | 35.464 | 0 | 0 | 0 | 0 | 0 |
| TKIDN1000164 | 0 | 22.887 | 0 | 0 | 0 | 0 | 0 |
| TKIDN1000171 | 0 | 100 | 0 | 0 | 0 | 0 | 0 |
| TKIDN1000192 | 0 | 20.450 | 0 | 0 | 0 | 12.377 | 0 |
| TKIDN2000127 | 0 | 67.072 | 0 | 0 | 0 | 0 | 0 |
| TKIDN2000240 | 0 | 100 | 0 | 0 | 0 | 0 | 0 |
| TKIDN2000246 | 0 | 7.882 | 0 | 0 | 0 | 0 | 0 |
| TKIDN2000319 | 0 | 78.393 | 0 | 0 | 0 | 0 | 0 |
| TKIDN2000464 | 0 | 68.029 | 0 | 0 | 0 | 0 | 0 |
| TKIDN2000521 | 0 | 55.069 | 0 | 0 | 0 | 0 | 0 |
| TKIDN2000701 | 0 | 84.991 | 0 | 0 | 0 | 0 | 0 |
| TKIDN2001529 | 0 | 74.873 | 0 | 0 | 0 | 0 | 0 |
| TKIDN2001662 | 18.188 | 40.370 | 0 | 0 | 0 | 0 | 0 |
| TKIDN2002318 | 0 | 39.706 | 0 | 0 | 0 | 0 | 0 |
| TKIDN2002329 | 0 | 100 | 0 | 0 | 0 | 0 | 0 |
| TKIDN2002424 | 0 | 100 | 0 | 0 | 0 | 0 | 0 |
| TKIDN2002632 | 0 | 100 | 0 | 0 | 0 | 0 | 0 |
| TKIDN2002738 | 0 | 1.552 | 0 | 0 | 0 | 0 | 0 |
| TKIDN2003044 | 0 | 100 | 0 | 0 | 0 | 0 | 0 |
| TKIDN2003059 | 0 | 78.393 | 0 | 0 | 0 | 0 | 0 |
| TKIDN2003062 | 0 | 100 | 0 | 0 | 0 | 0 | 0 |
| TKIDN2003078 | 0 | 51.264 | 0 | 0 | 0 | 0 | 0 |
| TKIDN2003366 | 0 | 100 | 0 | 0 | 0 | 0 | 0 |
| TKIDN2003396 | 0 | 100 | 0 | 0 | 0 | 0 | 0 |
| TKIDN2003413 | 0 | 100 | 0 | 0 | 0 | 0 | 0 |
| TKIDN2003597 | 0 | 28.918 | 0 | 0 | 0 | 0 | 0 |
| TKIDN2003601 | 15.897 | 70.571 | 0 | 0 | 0 | 0 | 0 |
| TKIDN2004185 | 0 | 14.684 | 0 | 0 | 0 | 0 | 0 |
| TKIDN2004386 | 0 | 100 | 0 | 0 | 0 | 0 | 0 |
| TKIDN2004458 | 0 | 100 | 0 | 0 | 0 | 0 | 0 |
| TKIDN2004748 | 0 | 35.796 | 0 | 0 | 0 | 0 | 0 |
| TKIDN2005487 | 0 | 2.659 | 0 | 0 | 0 | 0 | 0 |
| TKIDN2005721 | 0 | 100 | 0 | 0 | 0 | 0 | 0 |
| TKIDN2005934 | 0 | 100 | 0 | 0 | 0 | 0 | 0 |
| TKIDN2005947 | 0 | 100 | 0 | 0 | 0 | 0 | 0 |
| TKIDN2005956 | 0 | 100 | 0 | 0 | 0 | 0 | 0 |
| TKIDN2006525 | 0 | 100 | 0 | 0 | 0 | 0 | 0 |
| TKIDN2006761 | 0 | 78.604 | 0 | 0 | 0 | 0 | 0 |
| TKIDN2006852 | 0 | 100 | 0 | 0 | 0 | 0 | 0 |
| TKIDN2007605 | 10.416 | 46.241 | 0 | 0 | 0 | 0 | 0 |
| TKIDN2007667 | 0 | 100 | 0 | 0 | 0 | 0 | 0 |
| TKIDN2007828 | 0 | 24.698 | 0 | 0 | 0 | 0 | 0 |
| TKIDN2008176 | 0 | 78.604 | 0 | 0 | 0 | 0 | 0 |
| TKIDN2008778 | 0 | 100 | 0 | 0 | 0 | 0 | 0 |
| TKIDN2009092 | 0 | 100 | 0 | 0 | 0 | 0 | 0 |
| TKIDN2009330 | 0 | 76.948 | 0 | 0 | 0 | 0 | 0 |
| TKIDN2009481 | 0 | 100 | 0 | 0 | 0 | 0 | 0 |
| TKIDN2009641 | 0 | 21.402 | 0 | 0 | 0 | 0 | 0 |
| TKIDN2009794 | 0 | 53.409 | 0 | 0 | 0 | 16.162 | 0 |
| TKIDN2009889 | 0 | 64.923 | 0 | 0 | 0 | 0 | 0 |
| TKIDN2010200 | 0 | 100 | 0 | 0 | 0 | 0 | 0 |
| TKIDN2010232 | 0 | 100 | 0 | 0 | 0 | 0 | 0 |
| TKIDN2010278 | 0 | 100 | 0 | 0 | 0 | 0 | 0 |
| TKIDN2010602 | 0 | 100 | 0 | 0 | 0 | 0 | 0 |
| TKIDN2010812 | 0 | 39.464 | 0 | 0 | 0 | 0 | 0 |
| TKIDN2010934 | 0 | 74.873 | 0 | 0 | 0 | 0 | 0 |
| TKIDN2011051 | 0 | 100 | 0 | 0 | 0 | 0 | 0 |
| TKIDN2011160 | 0 | 100 | 0 | 0 | 0 | 0 | 0 |
| TKIDN2011289 | 0 | 22.129 | 0 | 0 | 0 | 0 | 0 |
| TKIDN2011324 | 0 | 100 | 0 | 0 | 0 | 0 | 0 |
| TKIDN2012034 | 0 | 100 | 0 | 0 | 0 | 0 | 0 |
| TKIDN2012771 | 0 | 100 | 0 | 0 | 0 | 0 | 0 |
| TKIDN2012824 | 0 | 100 | 0 | 0 | 0 | 0 | 0 |
| TKIDN2013134 | 0 | 100 | 0 | 0 | 0 | 0 | 0 |
| TKIDN2013287 | 14.432 | 64.067 | 0 | 0 | 0 | 0 | 0 |
| TKIDN2014570 | 0 | 78.731 | 0 | 0 | 0 | 0 | 0 |
| TKIDN2014757 | 0 | 100 | 0 | 0 | 0 | 0 | 0 |
| TKIDN2014771 | 0 | 100 | 0 | 0 | 0 | 0 | 0 |
| TKIDN2014964 | 0 | 40.568 | 0 | 0 | 0 | 0 | 0 |
| TKIDN2015025 | 0 | 100 | 0 | 0 | 0 | 0 | 0 |
| TKIDN2015071 | 0 | 48.016 | 0 | 0 | 0 | 0 | 0 |
| TKIDN2015161 | 0 | 100 | 0 | 0 | 0 | 0 | 0 |
| TKIDN2015263 | 0 | 100 | 0 | 0 | 0 | 0 | 0 |
| TKIDN2015285 | 0 | 36.749 | 0 | 0 | 0 | 0 | 0 |
| TKIDN2015423 | 0.531 | 2.357 | 0 | 0 | 0 | 0 | 0 |
| TKIDN2015788 | 2.193 | 9.737 | 0 | 0 | 0 | 2.946 | 0 |
| TKIDN2016309 | 0 | 100 | 0 | 0 | 0 | 0 | 0 |
| TKIDN2016399 | 0 | 34.998 | 65.002 | 0 | 0 | 0 | 0 |
| TKIDN2016846 | 0 | 83.911 | 0 | 0 | 0 | 0 | 0 |
| TKIDN2018926 | 0 | 78.604 | 0 | 0 | 0 | 0 | 0 |
| TKIDN2019116 | 0 | 100 | 0 | 0 | 0 | 0 | 0 |
| TLIVE2000023 | 0 | 0 | 100 | 0 | 0 | 0 | 0 |
| TLIVE2000142 | 0 | 0 | 100 | 0 | 0 | 0 | 0 |
| TLIVE2000292 | 0 | 0 | 100 | 0 | 0 | 0 | 0 |
| TLIVE2000762 | 0 | 0 | 100 | 0 | 0 | 0 | 0 |
| TLIVE2000979 | 0 | 0 | 73.389 | 0 | 0 | 0 | 0 |
| TLIVE2001327 | 0 | 0 | 64.491 | 0 | 0 | 0 | 0 |
| TLIVE2001616 | 0 | 0 | 100 | 0 | 0 | 0 | 0 |
| TLIVE2001684 | 0 | 0 | 100 | 0 | 0 | 0 | 0 |
| TLIVE2001828 | 10.817 | 0 | 89.183 | 0 | 0 | 0 | 0 |
| TLIVE2001927 | 0 | 0 | 100 | 0 | 0 | 0 | 0 |
| TLIVE2002046 | 3.027 | 0 | 12.478 | 0 | 0 | 2.033 | 0 |
| TLIVE2002336 | 0 | 0 | 100 | 0 | 0 | 0 | 0 |
| TLIVE2002338 | 0 | 0 | 100 | 0 | 0 | 0 | 0 |
| TLIVE2002562 | 0 | 0 | 100 | 0 | 0 | 0 | 0 |
| TLIVE2002690 | 0 | 0 | 41.431 | 0 | 0 | 0 | 0 |
| TLIVE2002882 | 0 | 0 | 100 | 0 | 0 | 0 | 0 |
| TLIVE2003197 | 0 | 0 | 100 | 0 | 0 | 0 | 0 |
| TLIVE2003200 | 0 | 0 | 87.078 | 0 | 0 | 0 | 0 |
| TLIVE2003225 | 0 | 0 | 100 | 0 | 0 | 0 | 0 |
| TLIVE2003381 | 0 | 0 | 100 | 0 | 0 | 0 | 0 |
| TLIVE2003737 | 0 | 0 | 100 | 0 | 0 | 0 | 0 |
| TLIVE2003961 | 0 | 0 | 100 | 0 | 0 | 0 | 0 |
| TLIVE2003970 | 0 | 0 | 15.901 | 0 | 0 | 5.181 | 0 |
| TLIVE2004110 | 0 | 0 | 70.982 | 0 | 0 | 0 | 0 |
| TLIVE2004320 | 0 | 0 | 87.217 | 0 | 0 | 0 | 0 |
| TLIVE2004331 | 0 | 0 | 100 | 0 | 0 | 0 | 0 |
| TLIVE2004601 | 0 | 0 | 100 | 0 | 0 | 0 | 0 |
| TLIVE2005180 | 0 | 0 | 100 | 0 | 0 | 0 | 0 |
| TLIVE2005390 | 0 | 0 | 100 | 0 | 0 | 0 | 0 |
| TLIVE2005516 | 0 | 0 | 100 | 0 | 0 | 0 | 0 |
| TLIVE2005731 | 0 | 0 | 100 | 0 | 0 | 0 | 0 |
| TLIVE2005866 | 0 | 0 | 100 | 0 | 0 | 0 | 0 |
| TLIVE2006236 | 0 | 0 | 100 | 0 | 0 | 0 | 0 |
| TLIVE2006359 | 0 | 0 | 100 | 0 | 0 | 0 | 0 |
| TLIVE2006529 | 0 | 0 | 100 | 0 | 0 | 0 | 0 |
| TLIVE2006621 | 0 | 0 | 100 | 0 | 0 | 0 | 0 |
| TLIVE2006733 | 0 | 0 | 100 | 0 | 0 | 0 | 0 |
| TLIVE2007000 | 0 | 0 | 100 | 0 | 0 | 0 | 0 |
| TLIVE2007016 | 0 | 0 | 100 | 0 | 0 | 0 | 0 |
| TLIVE2007132 | 0 | 0 | 100 | 0 | 0 | 0 | 0 |
| TLIVE2007192 | 0 | 0 | 100 | 0 | 0 | 0 | 0 |
| TLIVE2007528 | 0 | 0 | 100 | 0 | 0 | 0 | 0 |
| TLIVE2007607 | 0 | 0 | 100 | 0 | 0 | 0 | 0 |
| TLIVE2007736 | 0 | 0 | 90.642 | 0 | 0 | 0 | 0 |
| TLIVE2007816 | 0 | 0 | 100 | 0 | 0 | 0 | 0 |
| TLIVE2007919 | 0 | 0 | 100 | 0 | 0 | 0 | 0 |
| TLIVE2008083 | 0 | 0 | 100 | 0 | 0 | 0 | 0 |
| TLIVE2008213 | 0 | 0 | 100 | 0 | 0 | 0 | 0 |
| TLIVE2008221 | 0 | 0 | 100 | 0 | 0 | 0 | 0 |
| TLIVE2008229 | 0 | 0 | 91.317 | 0 | 0 | 0 | 0 |
| TLIVE2008712 | 0 | 0 | 100 | 0 | 0 | 0 | 0 |
| TLIVE2008797 | 0 | 0 | 100 | 0 | 0 | 0 | 0 |
| TLIVE2009087 | 0 | 0 | 100 | 0 | 0 | 0 | 0 |
| TLIVE2009163 | 0 | 0 | 100 | 0 | 0 | 0 | 0 |
| TLIVE2009489 | 0 | 0 | 100 | 0 | 0 | 0 | 0 |
| TLIVE2009541 | 0 | 0 | 100 | 0 | 0 | 0 | 0 |
| TLUNG2000179 | 0 | 0 | 0 | 96.684 | 0 | 0 | 0 |
| TOVAR2000476 | 0 | 0 | 0 | 0 | 49.428 | 0 | 0 |
| TOVAR2000575 | 3.687 | 0 | 0 | 0 | 96.313 | 0 | 0 |
| TOVAR2000649 | 0 | 0 | 0 | 0 | 100 | 0 | 0 |
| TOVAR2000904 | 0 | 0 | 0 | 0 | 95.526 | 0 | 0 |
| TOVAR2001281 | 0 | 0 | 0 | 0 | 100 | 0 | 0 |
| TOVAR2001556 | 0 | 0 | 0 | 0 | 100 | 0 | 0 |
| TOVAR2001730 | 0 | 0 | 0 | 0 | 100 | 0 | 0 |
| TOVAR2002247 | 0 | 0 | 0 | 0 | 100 | 0 | 0 |
| TOVAR2002514 | 0 | 0 | 0 | 0 | 100 | 0 | 0 |
| TOVAR2002549 | 0 | 0 | 0 | 0 | 100 | 0 | 0 |
| TOVAR2002800 | 0 | 0 | 0 | 0 | 100 | 0 | 0 |
| TRACH1000011 | 0 | 0 | 0 | 0 | 0 | 8.245 | 0 |
| TRACH1000018 | 0 | 0 | 0 | 0 | 0 | 23.133 | 0 |
| TRACH1000030 | 2.876 | 5.107 | 4.743 | 0 | 0 | 0.773 | 0 |
| TRACH1000038 | 0 | 0 | 0 | 0 | 0 | 21.094 | 0 |
| TRACH1000057 | 0 | 0 | 0 | 0 | 0 | 100 | 0 |
| TRACH1000063 | 0 | 0 | 0 | 0 | 0 | 2.409 | 0 |
| TRACH1000074 | 0 | 0 | 0 | 0 | 0 | 100 | 0 |
| TRACH1000100 | 0 | 0 | 0 | 0 | 0 | 100 | 0 |
| TRACH1000106 | 0 | 0 | 0 | 0 | 0 | 6.650 | 0 |
| TRACH1000125 | 42.674 | 0 | 0 | 0 | 0 | 57.326 | 0 |
| TRACH1000140 | 0 | 0 | 0 | 0 | 0 | 100 | 0 |
| TRACH1000144 | 0 | 0 | 0 | 0 | 0 | 100 | 0 |
| TRACH1000181 | 0 | 0 | 0 | 0 | 0 | 5.307 | 0 |
| TRACH1000193 | 0 | 0 | 0 | 0 | 0 | 48.662 | 0 |
| TRACH1000205 | 0 | 0 | 0 | 0 | 0 | 8.816 | 0 |
| TRACH1000212 | 0 | 0 | 0 | 0 | 0 | 100 | 0 |
| TRACH1000240 | 0 | 0 | 0 | 0 | 0 | 100 | 0 |
| TRACH2000002 | 0 | 0 | 0 | 0 | 0 | 100 | 0 |
| TRACH2000015 | 0 | 0 | 0 | 0 | 0 | 100 | 0 |
| TRACH2000024 | 0 | 0 | 0 | 0 | 0 | 100 | 0 |
| TRACH2000032 | 27.125 | 0 | 0 | 0 | 0 | 72.875 | 0 |
| TRACH2000045 | 0 | 0 | 0 | 0 | 0 | 6.160 | 0 |
| TRACH2000047 | 4.466 | 0 | 0 | 0 | 0 | 11.998 | 0 |
| TRACH2000079 | 0 | 2.005 | 0 | 0 | 0 | 2.427 | 0 |
| TRACH2000081 | 0 | 0 | 0 | 0 | 0 | 14.258 | 0 |
| TRACH2000083 | 0 | 0 | 0 | 0 | 0 | 100 | 0 |
| TRACH2000148 | 0 | 0 | 0 | 0 | 0 | 100 | 0 |
| TRACH2000185 | 0 | 0 | 0 | 0 | 0 | 100 | 0 |
| TRACH2000196 | 0 | 0 | 0 | 0 | 0 | 14.774 | 0 |
| TRACH2000235 | 0 | 0 | 0 | 0 | 0 | 100 | 0 |
| TRACH2000237 | 0 | 0 | 0 | 0 | 0 | 6.022 | 0 |
| TRACH2000243 | 0 | 0 | 0 | 0 | 0 | 0.346 | 0 |
| TRACH2000248 | 0 | 0 | 0 | 0 | 0 | 3.126 | 0 |
| TRACH2000250 | 15.327 | 0 | 0 | 0 | 0 | 20.590 | 0 |
| TRACH2000259 | 0 | 0 | 0 | 0 | 0 | 100 | 0 |
| TRACH2000272 | 0 | 0 | 0 | 0 | 0 | 100 | 0 |
| TRACH2000282 | 0 | 0 | 0 | 0 | 0 | 39.169 | 0 |
| TRACH2000287 | 0 | 0 | 0 | 0 | 0 | 100 | 0 |
| TRACH2000289 | 0 | 0 | 0 | 0 | 0 | 100 | 0 |
| TRACH2000306 | 0 | 0 | 77.769 | 0 | 0 | 6.335 | 0 |
| TRACH2000312 | 0 | 0 | 0 | 0 | 0 | 100 | 0 |
| TRACH2000321 | 1.639 | 0 | 3.378 | 0 | 0 | 1.651 | 0 |
| TRACH2000336 | 0 | 0 | 0 | 0 | 0 | 100 | 0 |
| TRACH2000359 | 0 | 11.039 | 0 | 0 | 0 | 13.362 | 0 |
| TRACH2000393 | 0 | 0 | 0 | 0 | 0 | 38.115 | 0 |
| TRACH2000411 | 0 | 0 | 0 | 0 | 0 | 100 | 0 |
| TRACH2000420 | 0 | 0 | 0 | 0 | 0 | 45.073 | 0 |
| TRACH2000461 | 4.289 | 0 | 0 | 0 | 0 | 5.762 | 0 |
| TRACH2000472 | 0.712 | 0 | 0 | 0 | 0 | 1.913 | 0 |
| TRACH2000496 | 0 | 0 | 0 | 0 | 0 | 100 | 0 |
| TRACH2000497 | 0 | 0 | 0 | 0 | 0 | 100 | 0 |
| TRACH2000502 | 0 | 0 | 0 | 0 | 0 | 52.833 | 0 |
| TRACH2000522 | 0 | 0 | 0 | 0 | 0 | 100 | 0 |
| TRACH2000540 | 2.376 | 0 | 0 | 0 | 0 | 1.596 | 0 |
| TRACH2000559 | 0 | 0 | 0 | 0 | 0 | 4.362 | 0 |
| TRACH2000660 | 21.151 | 0 | 0 | 0 | 0 | 28.413 | 0 |
| TRACH2000665 | 0 | 0 | 0 | 0 | 0 | 100 | 0 |
| TRACH2000675 | 0 | 0 | 0 | 0 | 0 | 100 | 0 |
| TRACH2000677 | 0 | 0 | 0 | 0 | 0 | 31.577 | 0 |
| TRACH2000702 | 0 | 0 | 0 | 0 | 0 | 4.398 | 0 |
| TRACH2000703 | 0 | 0 | 0 | 0 | 0 | 100 | 0 |
| TRACH2000767 | 59.821 | 0 | 0 | 0 | 0 | 40.179 | 0 |
| TRACH2000775 | 0 | 0 | 0 | 0 | 0 | 100 | 0 |
| TRACH2000780 | 2.186 | 0 | 0 | 0 | 0 | 7.832 | 0 |
| TRACH2000807 | 0 | 0 | 0 | 0 | 0 | 100 | 0 |
| TRACH2000862 | 0 | 0 | 0 | 0 | 0 | 6.484 | 0 |
| TRACH2000873 | 0 | 0 | 0 | 0 | 0 | 52.645 | 0 |
| TRACH2000894 | 0 | 0 | 0 | 0 | 0 | 100 | 0 |
| TRACH2000898 | 0 | 0 | 0 | 0 | 0 | 100 | 0 |
| TRACH2000920 | 2.764 | 0 | 0 | 0 | 0 | 3.713 | 0 |
| TRACH2000923 | 0 | 0 | 0 | 0 | 0 | 100 | 0 |
| TRACH2000926 | 0 | 0 | 0 | 0 | 0 | 3.125 | 0 |
| TRACH2000944 | 0 | 0 | 0 | 0 | 0 | 100 | 0 |
| TRACH2000959 | 0.611 | 0 | 0 | 0 | 0 | 0.273 | 0 |
| TRACH2000972 | 0 | 0 | 0 | 0 | 0 | 0.664 | 0 |
| TRACH2000981 | 0 | 0 | 0 | 0 | 0 | 100 | 0 |
| TRACH2000986 | 0 | 0 | 0 | 0 | 0 | 100 | 0 |
| TRACH2001021 | 0.698 | 0 | 5.757 | 0 | 0 | 0.938 | 8.992 |
| TRACH2001037 | 0 | 0 | 0 | 0 | 0 | 100 | 0 |
| TRACH2001060 | 0 | 0 | 0 | 0 | 0 | 37.554 | 0 |
| TRACH2001085 | 0 | 0 | 0 | 0 | 0 | 23.133 | 0 |
| TRACH2001101 | 0 | 0 | 0 | 0 | 0 | 23.819 | 0 |
| TRACH2001109 | 0 | 0.775 | 0 | 0 | 0 | 0.234 | 0 |
| TRACH2001121 | 0 | 0 | 0 | 0 | 0 | 100 | 0 |
| TRACH2001147 | 0 | 0 | 0 | 0 | 0 | 100 | 0 |
| TRACH2001154 | 0 | 0 | 0 | 0 | 0 | 100 | 0 |
| TRACH2001188 | 0 | 0 | 0 | 0 | 0 | 100 | 0 |
| TRACH2001192 | 0 | 0 | 0 | 0 | 0 | 37.759 | 0 |
| TRACH2001249 | 0 | 0 | 0 | 0 | 0 | 30.816 | 0 |
| TRACH2001275 | 0 | 0 | 0 | 0 | 0 | 100 | 0 |
| TRACH2001289 | 0.567 | 0 | 0 | 0 | 0 | 2.665 | 0 |
| TRACH2001314 | 1.524 | 0 | 0 | 0 | 0 | 8.190 | 0 |
| TRACH2001335 | 0 | 0 | 0 | 0 | 0 | 100 | 0 |
| TRACH2001339 | 0 | 0 | 0 | 0 | 0 | 32.807 | 0 |
| TRACH2001341 | 0 | 0 | 0 | 0 | 0 | 7.139 | 0 |
| TRACH2001347 | 0 | 0 | 0 | 0 | 0 | 100 | 0 |
| TRACH2001395 | 0 | 0 | 0 | 0 | 0 | 100 | 0 |
| TRACH2001400 | 0 | 0 | 0 | 0 | 0 | 100 | 0 |
| TRACH2001403 | 0 | 0 | 0 | 0 | 0 | 100 | 0 |
| TRACH2001416 | 0 | 0 | 0 | 0 | 0 | 100 | 0 |
| TRACH2001428 | 2.105 | 0 | 0 | 0 | 0 | 1.414 | 0 |
| TRACH2001432 | 0 | 4.118 | 7.648 | 0 | 0 | 1.246 | 0 |
| TRACH2001443 | 0 | 34.396 | 0 | 0 | 0 | 20.817 | 0 |
| TRACH2001463 | 2.553 | 0 | 0 | 0 | 0 | 3.430 | 0 |
| TRACH2001474 | 0 | 0 | 0 | 0 | 0 | 100 | 0 |
| TRACH2001478 | 0.968 | 0 | 0 | 0 | 0 | 3.251 | 0 |
| TRACH2001523 | 0 | 0 | 0 | 0 | 0 | 1.904 | 0 |
| TRACH2001525 | 0 | 0 | 0 | 0 | 0 | 100 | 0 |
| TRACH2001532 | 0 | 0 | 0 | 0 | 0 | 100 | 0 |
| TRACH2001549 | 3.526 | 0 | 0 | 0 | 0 | 23.681 | 0 |
| TRACH2001592 | 0 | 0 | 0 | 0 | 0 | 100 | 0 |
| TRACH2001596 | 17.065 | 0 | 0 | 0 | 0 | 22.924 | 0 |
| TRACH2001607 | 3.350 | 0 | 0 | 0 | 0 | 1.125 | 0 |
| TRACH2001612 | 0 | 0 | 0 | 0 | 0 | 4.474 | 0 |
| TRACH2001621 | 0 | 0 | 0 | 0 | 0 | 100 | 0 |
| TRACH2001646 | 0 | 0 | 0 | 0 | 0 | 25.331 | 0 |
| TRACH2001651 | 0 | 0 | 0 | 0 | 0 | 100 | 0 |
| TRACH2001684 | 0 | 0 | 0 | 0 | 0 | 19.952 | 0 |
| TRACH2001810 | 1.295 | 0 | 0 | 0 | 0 | 4.349 | 0 |
| TRACH2001898 | 0 | 0 | 0 | 0 | 0 | 1.142 | 0 |
| TRACH2001933 | 0 | 0 | 0 | 0 | 0 | 100 | 0 |
| TRACH2001968 | 0 | 0 | 0 | 0 | 0 | 100 | 0 |
| TRACH2001996 | 0 | 0 | 0 | 0 | 0 | 32.807 | 0 |
| TRACH2002043 | 0 | 0 | 0 | 0 | 0 | 1.188 | 0 |
| TRACH2002054 | 0 | 0 | 0 | 0 | 0 | 48.432 | 0 |
| TRACH2002092 | 0 | 0 | 0 | 0 | 0 | 3.144 | 0 |
| TRACH2002100 | 0 | 0 | 0 | 0 | 0 | 19.210 | 0 |
| TRACH2002138 | 0 | 0 | 0 | 0 | 0 | 24.095 | 0 |
| TRACH2002333 | 0 | 0 | 0 | 0 | 0 | 16.466 | 0 |
| TRACH2002537 | 0 | 0 | 6.376 | 0 | 0 | 2.078 | 0 |
| TRACH2002664 | 0 | 0 | 0 | 0 | 0 | 6.819 | 0 |
| TRACH2002784 | 0 | 0 | 0 | 0 | 0 | 100 | 0 |
| TRACH2002803 | 0 | 7.714 | 0 | 0 | 0 | 23.342 | 0 |
| TRACH2002840 | 0 | 0 | 0 | 0 | 0 | 100 | 0 |
| TRACH2002847 | 0 | 0 | 0 | 0 | 0 | 5.084 | 0 |
| TRACH2002853 | 0 | 0 | 0 | 0 | 0 | 100 | 0 |
| TRACH2002862 | 0 | 0 | 0 | 0 | 0 | 100 | 0 |
| TRACH2002954 | 0 | 0 | 0 | 0 | 0 | 100 | 0 |
| TRACH2002960 | 0 | 0 | 0 | 0 | 0 | 100 | 0 |
| TRACH2002988 | 42.674 | 0 | 0 | 0 | 0 | 57.326 | 0 |
| TRACH2003070 | 42.674 | 0 | 0 | 0 | 0 | 57.326 | 0 |
| TRACH2003272 | 6.179 | 0 | 0 | 0 | 4.150 | 0 | |
| TRACH2003323 | 0 | 0 | 0 | 0 | 0 | 7.974 | 0 |
| TRACH2003347 | 1.325 | 5.881 | 0 | 0 | 0 | 5.339 | 34.119 |
| TRACH2003484 | 0 | 0 | 0 | 0 | 0 | 61.213 | 0 |
| TRACH2003516 | 0 | 0 | 0 | 0 | 0 | 100 | 0 |
| TRACH2003605 | 42.674 | 0 | 0 | 0 | 0 | 57.326 | 0 |
| TRACH2003736 | 0 | 0 | 0 | 0 | 0 | 100 | 0 |
| TRACH2003759 | 0 | 0 | 0 | 0 | 0 | 100 | 0 |
| TRACH2003941 | 0 | 0 | 0 | 0 | 0 | 100 | 0 |
| TRACH2004039 | 0 | 0 | 0 | 0 | 0 | 63.148 | 0 |
| TRACH2004054 | 0 | 0 | 0 | 0 | 0 | 100 | 0 |
| TRACH2004087 | 0 | 0 | 0 | 0 | 0 | 100 | 0 |
| TRACH2004109 | 0 | 0 | 0 | 0 | 0 | 39.169 | 0 |
| TRACH2004170 | 0 | 0 | 0 | 0 | 0 | 100 | 0 |
| TRACH2004183 | 0 | 0 | 0 | 0 | 0 | 100 | 0 |
| TRACH2004251 | 0 | 0 | 0 | 0 | 0 | 100 | 0 |
| TRACH2004292 | 0 | 0 | 0 | 0 | 0 | 100 | 0 |
| TRACH2004336 | 0 | 0 | 0 | 0 | 0 | 63.148 | 0 |
| TRACH2004399 | 24.718 | 0 | 0 | 0 | 0 | 33.204 | 0 |
| TRACH2004428 | 0 | 0 | 0 | 0 | 0 | 100 | 0 |
| TRACH2004458 | 0 | 0 | 0 | 0 | 0 | 23.664 | 0 |
| TRACH2004499 | 0 | 0 | 0 | 0 | 0 | 3.292 | 0 |
| TRACH2004521 | 0 | 0 | 0 | 0 | 0 | 100 | 0 |
| TRACH2004570 | 0 | 0 | 0 | 0 | 0 | 100 | 0 |
| TRACH2004845 | 0 | 0 | 0 | 0 | 0 | 100 | 0 |
| TRACH2004887 | 0 | 0 | 0 | 0 | 0 | 100 | 0 |
| TRACH2004950 | 0 | 0 | 0 | 0 | 0 | 100 | 0 |
| TRACH2005004 | 0 | 0 | 0 | 0 | 0 | 100 | 0 |
| TRACH2005017 | 0 | 0 | 0 | 0 | 0 | 100 | 0 |
| TRACH2005066 | 0 | 0 | 0 | 0 | 0 | 100 | 0 |
| TRACH2005159 | 0 | 0 | 0 | 0 | 0 | 100 | 0 |
| TRACH2005314 | 0 | 0 | 0 | 0 | 0 | 100 | 0 |
| TRACH2005444 | 0 | 0 | 0 | 0 | 0 | 100 | 0 |
| TRACH2005541 | 0 | 0 | 0 | 0 | 0 | 10.892 | 0 |
| TRACH2005666 | 0 | 0 | 0 | 0 | 0 | 100 | 0 |
| TRACH2005698 | 42.674 | 0 | 0 | 0 | 0 | 57.326 | 0 |
| TRACH2005710 | 0 | 0 | 0 | 0 | 0 | 15.040 | 0 |
| TRACH2005720 | 0 | 0 | 0 | 0 | 0 | 100 | 0 |
| TRACH2005769 | 0 | 0 | 0 | 0 | 0 | 100 | 0 |
| TRACH2005796 | 0 | 0 | 0 | 0 | 0 | 50.251 | 0 |
| TRACH2005800 | 0 | 0 | 0 | 0 | 0 | 2.695 | 0 |
| TRACH2005811 | 4.397 | 0 | 0 | 0 | 0 | 3.544 | 22.648 |
| TRACH2006015 | 0 | 0 | 0 | 0 | 0 | 100 | 0 |
| TRACH2006049 | 0 | 0 | 0 | 0 | 0 | 52.833 | 0 |
| TRACH2006097 | 0 | 0 | 0 | 0 | 0 | 100 | 0 |
| TRACH2006194 | 0 | 0 | 0 | 0 | 0 | 100 | 0 |
| TRACH2006327 | 0 | 0 | 0 | 0 | 0 | 100 | 0 |
| TRACH2006331 | 0 | 0 | 0 | 0 | 0 | 100 | 0 |
| TRACH2006378 | 0 | 0 | 0 | 0 | 0 | 20.023 | 0 |
| TRACH2006387 | 0 | 0 | 0 | 0 | 0 | 100 | 0 |
| TRACH2006528 | 0 | 0 | 0 | 0 | 0 | 100 | 0 |
| TRACH2006670 | 0 | 0 | 0 | 0 | 0 | 100 | 0 |
| TRACH2006762 | 0 | 0 | 0 | 0 | 0 | 24.004 | 0 |
| TRACH2006866 | 0 | 0 | 0 | 0 | 0 | 5.558 | 21.311 |
| TRACH2006870 | 0 | 0 | 0 | 0 | 0 | 100 | 0 |
| TRACH2006918 | 0 | 0 | 0 | 0 | 0 | 100 | 0 |
| TRACH2007059 | 0 | 0 | 0 | 0 | 0 | 37.554 | 0 |
| TRACH2007324 | 0 | 0 | 0 | 0 | 0 | 100 | 0 |
| TRACH2007358 | 0 | 0 | 0 | 0 | 0 | 13.193 | 0 |
| TRACH2007399 | 0 | 0 | 0 | 0 | 0 | 3.693 | 0 |
| TRACH2007483 | 0 | 0 | 0 | 0 | 0 | 100 | 0 |
| TRACH2007542 | 0 | 0 | 0 | 0 | 0 | 100 | 0 |
| TRACH2007577 | 0 | 0 | 0 | 0 | 0 | 100 | 0 |
| TRACH2007674 | 0 | 0 | 0 | 0 | 0 | 100 | 0 |
| TRACH2007676 | 0 | 0 | 0 | 0 | 0 | 100 | 0 |
| TRACH2007688 | 0 | 0 | 0 | 0 | 0 | 100 | 0 |
| TRACH2007733 | 0 | 0 | 0 | 0 | 0 | 100 | 0 |
| TRACH2007754 | 0 | 0 | 30.987 | 0 | 0 | 1.683 | 0 |
| TRACH2007816 | 16.648 | 0 | 0 | 0 | 0 | 11.182 | 0 |
| TRACH2007894 | 42.674 | 0 | 0 | 0 | 0 | 57.326 | 0 |
| TRACH2007969 | 2.116 | 0 | 0 | 0 | 55.276 | 5.685 | 0 |
| TRACH2007988 | 0 | 0 | 0 | 0 | 0 | 100 | 0 |
| TRACH2008081 | 2.130 | 0 | 0 | 0 | 0 | 1.431 | 0 |
| TRACH2008112 | 0 | 0 | 0 | 0 | 0 | 100 | 0 |
| TRACH2008113 | 0 | 0 | 0 | 0 | 0 | 20.849 | 0 |
| TRACH2008127 | 0 | 0 | 0 | 0 | 0 | 100 | 0 |
| TRACH2008130 | 0 | 0 | 0 | 0 | 0 | 47.415 | 0 |
| TRACH2008278 | 0 | 0 | 0 | 0 | 0 | 100 | 0 |
| TRACH2008300 | 0 | 6.284 | 0 | 0 | 0 | 1.902 | 0 |
| TRACH2008472 | 6.542 | 0 | 0 | 0 | 0 | 3.515 | 0 |
| TRACH2008478 | 0 | 0 | 0 | 0 | 0 | 20.004 | 0 |
| TRACH2008540 | 0 | 0 | 0 | 0 | 0 | 17.670 | 0 |
| TRACH2008583 | 0 | 0 | 0 | 0 | 0 | 100 | 0 |
| TRACH2008597 | 42.674 | 0 | 0 | 0 | 0 | 57.326 | 0 |
| TRACH2008716 | 0 | 0 | 0 | 0 | 0 | 5.682 | 0 |
| TRACH2008820 | 0 | 0 | 0 | 0 | 0 | 100 | 0 |
| TRACH2008891 | 0 | 0 | 0 | 0 | 0 | 24.300 | 0 |
| TRACH2008921 | 0 | 0 | 0 | 0 | 0 | 100 | 0 |
| TRACH2009006 | 11.705 | 0 | 0 | 0 | 0 | 15.724 | 0 |
| TRACH2009060 | 0 | 0 | 0 | 0 | 0 | 100 | 0 |
| TRACH2009107 | 0 | 0 | 0 | 0 | 0 | 14.068 | 0 |
| TRACH2009123 | 0 | 0 | 0 | 0 | 0 | 100 | 0 |
| TRACH2009268 | 0 | 0 | 0 | 0 | 0 | 100 | 0 |
| TRACH2009291 | 0 | 0 | 0 | 0 | 0 | 23.395 | 0 |
| TRACH2009310 | 0 | 0 | 0 | 0 | 0 | 19.666 | 0 |
| TRACH2009340 | 8.253 | 0 | 0 | 0 | 0 | 11.087 | 0 |
| TRACH2009348 | 0 | 0 | 0 | 0 | 0 | 100 | 0 |
| TRACH2009358 | 0 | 0 | 0 | 0 | 0 | 100 | 0 |
| TRACH2009661 | 0 | 0 | 0 | 0 | 0 | 63.148 | 0 |
| TRACH2009730 | 0 | 0 | 0 | 0 | 0 | 100 | 0 |
| TRACH2009743 | 0 | 0 | 0 | 0 | 0 | 100 | 0 |
| TRACH2009754 | 0 | 0 | 0 | 0 | 0 | 100 | 0 |
| TRACH2009851 | 0 | 0 | 0 | 0 | 0 | 16.843 | 0 |
| TRACH2009862 | 0 | 0 | 0 | 0 | 0 | 100 | 0 |
| TRACH2009934 | 42.674 | 0 | 0 | 0 | 0 | 57.326 | 0 |
| TRACH2010159 | 0 | 0 | 0 | 0 | 0 | 100 | 0 |
| TRACH2010218 | 0 | 0 | 0 | 0 | 0 | 100 | 0 |
| TRACH2010251 | 0 | 0 | 0 | 0 | 0 | 100 | 0 |
| TRACH2010272 | 0 | 0 | 0 | 0 | 0 | 100 | 0 |
| TRACH2010317 | 42.674 | 0 | 0 | 0 | 0 | 57.326 | 0 |
| TRACH2010419 | 0 | 0 | 0 | 0 | 0 | 100 | 0 |
| TRACH2010451 | 0 | 0 | 0 | 0 | 0 | 100 | 0 |
| TRACH2010505 | 0 | 0 | 0 | 0 | 0 | 100 | 0 |
| TRACH2010587 | 0 | 0 | 0 | 0 | 0 | 100 | 0 |
| TRACH2010600 | 0 | 0 | 0 | 0 | 0 | 38.628 | 0 |
| TRACH2010634 | 0 | 0 | 0 | 0 | 0 | 38.834 | 0 |
| TRACH2010639 | 0 | 0 | 0 | 0 | 0 | 100 | 0 |
| TRACH2010677 | 0 | 0 | 0 | 0 | 0 | 100 | 0 |
| TRACH2010698 | 0 | 0 | 0 | 0 | 0 | 100 | 0 |
| TRACH2010771 | 0 | 0 | 0 | 0 | 0 | 38.134 | 0 |
| TRACH2010824 | 14.977 | 0 | 0 | 0 | 0 | 20.120 | 0 |
| TRACH2010883 | 0 | 0 | 0 | 0 | 0 | 100 | 0 |
| TRACH2010965 | 0 | 0 | 0 | 0 | 0 | 100 | 0 |
| TRACH2010974 | 0 | 0 | 0 | 0 | 0 | 100 | 0 |
| TRACH2011057 | 0 | 0 | 0 | 0 | 0 | 100 | 0 |
| TRACH2011093 | 0 | 0 | 0 | 0 | 0 | 100 | 0 |
| TRACH2011113 | 7.874 | 0 | 0 | 0 | 0 | 5.288 | 0 |
| TRACH2011290 | 0 | 0 | 0 | 0 | 0 | 47.415 | 0 |
| TRACH2011293 | 0 | 0 | 0 | 0 | 0 | 5.001 | 0 |
| TRACH2011302 | 0 | 0 | 0 | 0 | 0 | 13.725 | 0 |
| TRACH2011500 | 0 | 0 | 0 | 0 | 0 | 15.124 | 0 |
| TRACH2011574 | 0 | 0 | 0 | 0 | 0 | 100 | 0 |
| TRACH2011894 | 0 | 0 | 0 | 0 | 0 | 9.961 | 0 |
| TRACH2011956 | 0 | 0 | 0 | 0 | 0 | 100 | 0 |
| TRACH2012000 | 0 | 0 | 0 | 0 | 0 | 100 | 0 |
| TRACH2012138 | 0 | 0 | 0 | 0 | 0 | 100 | 0 |
| TRACH2012242 | 0 | 0 | 0 | 0 | 0 | 100 | 0 |
| TRACH2012298 | 0 | 0 | 0 | 0 | 0 | 1.589 | 0 |
| TRACH2012370 | 0 | 0 | 0 | 0 | 0 | 100 | 0 |
| TRACH2012376 | 0 | 0 | 0 | 0 | 0 | 3.006 | 0 |
| TRACH2012387 | 0 | 0 | 0 | 0 | 0 | 63.148 | 0 |
| TRACH2012497 | 0 | 0 | 0 | 0 | 0 | 7.251 | 0 |
| TRACH2012536 | 0 | 0 | 0 | 0 | 0 | 38.628 | 0 |
| TRACH2012562 | 0 | 0 | 0 | 0 | 0 | 100 | 0 |
| TRACH2012742 | 0 | 0 | 0 | 0 | 0 | 100 | 0 |
| TRACH2012811 | 0 | 0 | 0 | 0 | 0 | 8.733 | 0 |
| TRACH2012823 | 0 | 0 | 0 | 0 | 0 | 100 | 0 |
| TRACH2012918 | 0 | 0 | 0 | 0 | 0 | 100 | 0 |
| TRACH2013081 | 0 | 0 | 0 | 0 | 0 | 13.033 | 0 |
| TRACH2013123 | 0 | 0 | 0 | 0 | 0 | 26.942 | 0 |
| TRACH2013265 | 0 | 0 | 0 | 0 | 0 | 100 | 0 |
| TRACH2013369 | 0 | 0 | 0 | 0 | 0 | 100 | 0 |
| TRACH2013450 | 0 | 0 | 0 | 0 | 0 | 100 | 0 |
| TRACH2013495 | 0 | 0 | 0 | 0 | 0 | 12.564 | 0 |
| TRACH2013552 | 0 | 0 | 0 | 0 | 0 | 11.637 | 0 |
| TRACH2013585 | 0 | 0 | 0 | 0 | 0 | 52.582 | 0 |
| TRACH2013671 | 0 | 0 | 0 | 0 | 0 | 100 | 0 |
| TRACH2013726 | 0 | 0 | 0 | 0 | 0 | 100 | 0 |
| TRACH2013902 | 0 | 0 | 0 | 0 | 0 | 100 | 0 |
| TRACH2013928 | 0 | 0 | 0 | 0 | 0 | 100 | 0 |
| TRACH2013982 | 0 | 0 | 0 | 0 | 0 | 100 | 0 |
| TRACH2013991 | 0 | 0 | 0 | 0 | 0 | 14.258 | 0 |
| TRACH2014018 | 0 | 0 | 0 | 0 | 0 | 100 | 0 |
| TRACH2014045 | 0 | 0 | 0 | 0 | 0 | 100 | 0 |
| TRACH2014046 | 0 | 0 | 0 | 0 | 0 | 100 | 0 |
| TRACH2014077 | 0 | 0 | 0 | 0 | 0 | 11.431 | 0 |
| TRACH2014082 | 0 | 0 | 0 | 0 | 0 | 100 | 0 |
| TRACH2014124 | 9.468 | 0 | 0 | 0 | 0 | 1.156 | 0 |
| TRACH2014268 | 0 | 0 | 0 | 0 | 0 | 100 | 0 |
| TRACH2014284 | 0 | 0 | 0 | 0 | 0 | 100 | 0 |
| TRACH2014337 | 0 | 0 | 0 | 0 | 0 | 100 | 0 |
| TRACH2014356 | 0 | 0 | 0 | 0 | 0 | 100 | 0 |
| TRACH2014371 | 0 | 0 | 0 | 0 | 0 | 100 | 0 |
| TRACH2014442 | 0 | 0 | 0 | 0 | 0 | 2.522 | 0 |
| TRACH2014483 | 0 | 0 | 0 | 0 | 0 | 100 | 0 |
| TRACH2014495 | 0 | 0 | 0 | 0 | 0 | 100 | 0 |
| TRACH2014544 | 0 | 0 | 0 | 0 | 0 | 100 | 0 |
| TRACH2014573 | 0 | 0 | 0 | 0 | 0 | 100 | 0 |
| TRACH2014725 | 0 | 0 | 0 | 0 | 0 | 100 | 0 |
| TRACH2014760 | 0 | 0 | 0 | 0 | 0 | 100 | 0 |
| TRACH2014815 | 0 | 0 | 0 | 0 | 0 | 100 | 0 |
| TRACH2014839 | 0 | 0 | 0 | 0 | 0 | 100 | 0 |
| TRACH2014938 | 0 | 0 | 0 | 0 | 0 | 100 | 0 |
| TRACH2014950 | 0 | 0 | 0 | 0 | 0 | 100 | 0 |
| TRACH2014972 | 0 | 0 | 0 | 0 | 0 | 100 | 0 |
| TRACH2014974 | 0 | 0 | 0 | 0 | 0 | 100 | 0 |
| TRACH2014997 | 0 | 0 | 0 | 0 | 0 | 15.851 | 0 |
| TRACH2015180 | 0 | 0 | 0 | 0 | 0 | 100 | 0 |
| TRACH2015243 | 0 | 0 | 0 | 0 | 0 | 100 | 0 |
| TRACH2015316 | 0 | 0 | 0 | 0 | 0 | 100 | 0 |
| TRACH2015381 | 0 | 0 | 0 | 0 | 0 | 100 | 0 |
| TRACH2015486 | 0 | 0 | 0 | 0 | 0 | 100 | 0 |
| TRACH2015654 | 0 | 0 | 0 | 0 | 0 | 23.395 | 0 |
| TRACH2015823 | 10.944 | 0 | 0 | 0 | 0 | 7.351 | 0 |
| TRACH2015824 | 0 | 0 | 0 | 0 | 0 | 2.603 | 0 |
| TRACH2015939 | 14.052 | 0 | 0 | 0 | 0 | 18.877 | 0 |
| TRACH2015987 | 0 | 0 | 0 | 0 | 0 | 100 | 0 |
| TRACH2016080 | 0 | 0 | 0 | 0 | 0 | 100 | 0 |
| TRACH2016131 | 0 | 0 | 0 | 0 | 0 | 32.807 | 0 |
| TRACH2016286 | 0 | 0 | 0 | 0 | 0 | 100 | 0 |
| TRACH2016317 | 0 | 0 | 0 | 0 | 0 | 17.467 | 0 |
| TRACH2016347 | 0 | 0 | 0 | 0 | 0 | 100 | 0 |
| TRACH2016380 | 0 | 0 | 0 | 0 | 0 | 100 | 0 |
| TRACH2016384 | 0 | 0 | 0 | 0 | 0 | 100 | 0 |
| TRACH2016410 | 0 | 0 | 0 | 0 | 0 | 100 | 0 |
| TRACH2016481 | 0 | 0 | 0 | 0 | 0 | 48.432 | 0 |
| TRACH2016498 | 0 | 0 | 0 | 0 | 0 | 100 | 0 |
| TRACH2016533 | 23.903 | 0 | 0 | 0 | 0 | 16.055 | 0 |
| TRACH2016551 | 0 | 0 | 0 | 0 | 0 | 100 | 0 |
| TRACH2016554 | 0 | 0 | 0 | 0 | 0 | 100 | 0 |
| TRACH2016651 | 0 | 0 | 0 | 0 | 0 | 100 | 0 |
| TRACH2016690 | 0 | 0 | 0 | 0 | 0 | 100 | 0 |
| TRACH2016709 | 0 | 0 | 0 | 0 | 0 | 100 | 0 |
| TRACH2016722 | 0 | 0 | 0 | 0 | 0 | 100 | 0 |
| TRACH2016835 | 15.389 | 0 | 0 | 0 | 0 | 20.672 | 0 |
| TRACH2016980 | 0 | 0 | 0 | 0 | 0 | 100 | 0 |
| TRACH2017086 | 0 | 0 | 0 | 0 | 0 | 100 | 0 |
| TRACH2017368 | 0 | 0 | 0 | 0 | 0 | 4.896 | 0 |
| TRACH2017498 | 0 | 0 | 0 | 0 | 0 | 100 | 0 |
| TRACH2017609 | 26.499 | 0 | 0 | 0 | 0 | 35.597 | 0 |
| TRACH2017949 | 0 | 0 | 0 | 0 | 0 | 100 | 0 |
| TRACH2017965 | 0 | 0 | 0 | 0 | 0 | 100 | 0 |
| TRACH2017980 | 0 | 0 | 0 | 0 | 0 | 100 | 0 |
| TRACH2018034 | 0 | 0 | 0 | 0 | 0 | 50.251 | 0 |
| TRACH2018084 | 0 | 0 | 0 | 0 | 0 | 100 | 0 |
| TRACH2018262 | 0 | 0 | 0 | 0 | 0 | 100 | 0 |
| TRACH2018273 | 0 | 0 | 0 | 0 | 0 | 100 | 0 |
| TRACH2018278 | 0 | 0 | 0 | 0 | 0 | 100 | 0 |
| TRACH2018317 | 0 | 10.844 | 0 | 0 | 0 | 3.281 | 0 |
| TRACH2018345 | 0 | 0 | 0 | 0 | 0 | 100 | 0 |
| TRACH2018380 | 0 | 0 | 0 | 0 | 0 | 100 | 0 |
| TRACH2018446 | 6.974 | 0 | 0 | 0 | 0 | 9.369 | 0 |
| TRACH2018449 | 0 | 56.123 | 0 | 0 | 0 | 33.966 | 0 |
| TRACH2018512 | 0 | 0 | 0 | 0 | 0 | 100 | 0 |
| TRACH2018646 | 0 | 0 | 0 | 0 | 0 | 100 | 0 |
| TRACH2018663 | 0 | 0 | 0 | 0 | 0 | 100 | 0 |
| TRACH2018718 | 0 | 0 | 0 | 0 | 0 | 100 | 0 |
| TRACH2018835 | 0 | 0 | 0 | 0 | 0 | 100 | 0 |
| TRACH2018882 | 0 | 0 | 0 | 0 | 0 | 100 | 0 |
| TRACH2018914 | 0 | 0 | 0 | 0 | 0 | 100 | 0 |
| TRACH2018950 | 0 | 0 | 0 | 0 | 0 | 8.413 | 0 |
| TRACH2019024 | 0 | 0 | 0 | 0 | 0 | 100 | 0 |
| TRACH2019046 | 0 | 0 | 0 | 0 | 0 | 100 | 0 |
| TRACH2019080 | 0 | 0 | 0 | 0 | 0 | 34.182 | 0 |
| TRACH2019151 | 0 | 0 | 0 | 0 | 0 | 100 | 0 |
| TRACH2019154 | 0 | 0 | 0 | 0 | 0 | 100 | 0 |
| TRACH2019176 | 0 | 0 | 0 | 0 | 0 | 100 | 0 |
| TRACH2019248 | 0 | 0 | 0 | 0 | 0 | 100 | 0 |
| TRACH2019309 | 0 | 0 | 0 | 0 | 0 | 100 | 0 |
| TRACH2019425 | 0 | 0 | 0 | 0 | 0 | 12.564 | 0 |
| TRACH2019473 | 0 | 0 | 0 | 0 | 0 | 100 | 0 |
| TRACH2019602 | 0 | 0 | 0 | 0 | 0 | 100 | 0 |
| TRACH2019661 | 0 | 0 | 0 | 0 | 0 | 100 | 0 |
| TRACH2019672 | 0 | 0 | 0 | 0 | 0 | 23.395 | 0 |
| TRACH2019673 | 0 | 0 | 0 | 0 | 0 | 100 | 0 |
| TRACH2019844 | 0 | 0 | 0 | 0 | 0 | 100 | 0 |
| TRACH2019879 | 0 | 0 | 0 | 0 | 0 | 100 | 0 |
| TRACH2020048 | 0 | 0 | 0 | 0 | 0 | 100 | 0 |
| TRACH2020336 | 0 | 0 | 0 | 0 | 0 | 100 | 0 |
| TRACH2020525 | 0 | 0 | 0 | 0 | 0 | 100 | 0 |
| TRACH2021398 | 0 | 0 | 0 | 0 | 0 | 100 | 0 |
| TRACH2021622 | 0 | 0 | 0 | 0 | 0 | 100 | 0 |
| TRACH2021635 | 0 | 0 | 0 | 0 | 0 | 100 | 0 |
| TRACH2021964 | 0 | 0 | 0 | 0 | 0 | 100 | 0 |
| TRACH2022042 | 0 | 0 | 0 | 0 | 0 | 100 | 0 |
| TRACH2022113 | 0 | 0 | 0 | 0 | 0 | 100 | 0 |
| TRACH2022253 | 0 | 0 | 0 | 0 | 0 | 100 | 0 |
| TRACH2022425 | 0 | 0 | 0 | 0 | 0 | 100 | 0 |
| TRACH2022553 | 0 | 0 | 0 | 0 | 0 | 100 | 0 |
| TRACH2022649 | 0 | 0 | 0 | 0 | 0 | 100 | 0 |
| TRACH2022839 | 0 | 0 | 0 | 0 | 0 | 100 | 0 |
| TRACH2023246 | 0 | 0 | 0 | 0 | 0 | 100 | 0 |
| TRACH2023299 | 0 | 0 | 0 | 0 | 0 | 7.150 | 0 |
| TRACH2023306 | 0 | 0 | 0 | 0 | 0 | 100 | 0 |
| TRACH2023416 | 0 | 0 | 0 | 0 | 0 | 100 | 0 |
| TRACH2023479 | 0 | 0 | 0 | 0 | 0 | 100 | 0 |
| TRACH2023684 | 0 | 0 | 0 | 0 | 0 | 100 | 0 |
| TRACH2023860 | 0 | 0 | 0 | 0 | 0 | 100 | 0 |
| TRACH2023876 | 0 | 0 | 0 | 0 | 0 | 100 | 0 |
| TRACH2024139 | 0 | 0 | 0 | 0 | 0 | 100 | 0 |
| TRACH2024187 | 0 | 0 | 0 | 0 | 0 | 100 | 0 |
| TRACH2024271 | 0 | 0 | 0 | 0 | 0 | 100 | 0 |
| TRACH2024293 | 53.839 | 0 | 0 | 0 | 0 | 18.081 | 0 |
| TRACH2024408 | 0 | 0 | 0 | 0 | 0 | 100 | 0 |
| TRACH2024512 | 0 | 0 | 0 | 0 | 0 | 100 | 0 |
| TRACH2024559 | 0 | 0 | 0 | 0 | 0 | 100 | 0 |
| TRACH2024647 | 0 | 0 | 0 | 0 | 0 | 100 | 0 |
| TRACH2024651 | 0 | 0 | 0 | 0 | 0 | 100 | 0 |
| TRACH2024730 | 0 | 0 | 0 | 0 | 0 | 100 | 0 |
| TRACH2024735 | 0 | 0 | 0 | 0 | 0 | 100 | 0 |
| TRACH2024749 | 0 | 0 | 0 | 0 | 0 | 100 | 0 |
| TRACH2024821 | 0 | 0 | 0 | 0 | 0 | 100 | 0 |
| TRACH2025057 | 0 | 0 | 0 | 0 | 0 | 100 | 0 |
| TRACH2025084 | 0 | 0 | 0 | 0 | 0 | 100 | 0 |
| TRACH2025155 | 0 | 0 | 0 | 0 | 0 | 100 | 0 |
| TRACH2025224 | 0 | 0 | 0 | 0 | 0 | 100 | 0 |
| TRACH2025344 | 0 | 0 | 0 | 0 | 0 | 11.031 | 0 |
| TRACH2025379 | 0 | 0 | 0 | 0 | 0 | 100 | 0 |
| TRACH2025507 | 0 | 0 | 0 | 0 | 0 | 100 | 0 |
| TRACH2025535 | 0.968 | 0 | 0 | 0 | 0 | 2.601 | 12.468 |
| TRACH2025569 | 0 | 0 | 0 | 0 | 0 | 100 | 0 |
| TRACH2025705 | 0 | 0 | 0 | 0 | 0 | 100 | 0 |
| TRACH2025749 | 0 | 0 | 0 | 0 | 0 | 100 | 0 |
| TRACH2025753 | 0 | 0 | 0 | 0 | 0 | 100 | 0 |
| TRACH2025810 | 0 | 0 | 0 | 0 | 0 | 100 | 0 |
| TRACH2025853 | 0 | 0 | 0 | 0 | 0 | 100 | 0 |
| TRACH2025911 | 0 | 0 | 0 | 0 | 0 | 52.645 | 0 |
| TRACH2025932 | 0 | 0 | 0 | 0 | 0 | 100 | 0 |
| TRACH3000014 | 0 | 0 | 0 | 0 | 0 | 81.453 | 0 |
| TRACH3000017 | 0 | 0 | 0 | 0 | 0 | 100 | 0 |
| TRACH3000043 | 0 | 0 | 0 | 0 | 0 | 100 | 0 |
| TRACH3000134 | 0 | 0 | 0 | 0 | 0 | 20.255 | 0 |
| TRACH3000180 | 0 | 0 | 0 | 0 | 0 | 100 | 0 |
| TRACH3000342 | 0 | 0 | 0 | 0 | 0 | 100 | 0 |
| TRACH3000420 | 0 | 0 | 0 | 0 | 0 | 100 | 0 |
| TRACH3000515 | 0 | 0 | 0 | 0 | 0 | 100 | 0 |
| TRACH3000530 | 0 | 0 | 0 | 0 | 0 | 100 | 0 |
| TRACH3000548 | 0 | 0 | 0 | 0 | 0 | 100 | 0 |
| TRACH3000558 | 0 | 0 | 0 | 0 | 0 | 100 | 0 |
| TRACH3000586 | 0 | 0 | 0 | 0 | 0 | 100 | 0 |
| TRACH3000692 | 0 | 0 | 0 | 0 | 0 | 15.750 | 0 |
| TRACH3000780 | 0 | 0 | 0 | 0 | 0 | 52.334 | 0 |
| TRACH3000892 | 0 | 0 | 0 | 0 | 0 | 100 | 0 |
| TRACH3000926 | 0 | 0 | 0 | 0 | 0 | 100 | 0 |
| TRACH3001119 | 0 | 0 | 0 | 0 | 0 | 100 | 0 |
| TRACH3001443 | 0 | 0 | 0 | 0 | 0 | 100 | 0 |
| TRACH3001550 | 0 | 0 | 0 | 0 | 0 | 52.334 | 0 |
| TRACH3001759 | 0 | 0 | 0 | 0 | 0 | 100 | 0 |
| TRACH3002064 | 0 | 0 | 0 | 0 | 0 | 100 | 0 |
| TRACH3002107 | 0 | 0 | 0 | 0 | 0 | 100 | 0 |
| TRACH3002168 | 0 | 0 | 0 | 0 | 0 | 100 | 0 |
| TRACH3002188 | 0 | 0 | 0 | 0 | 0 | 100 | 0 |
| TRACH3002192 | 2.600 | 0 | 0 | 0 | 0 | 3.493 | 0 |
| TRACH3002245 | 0 | 0 | 0 | 0 | 0 | 100 | 0 |
| TRACH3002293 | 0 | 0 | 0 | 0 | 0 | 100 | 0 |
| TRACH3002533 | 0 | 0 | 0 | 0 | 0 | 100 | 0 |
| TRACH3002561 | 0 | 0 | 0 | 0 | 0 | 100 | 0 |
| TRACH3002567 | 0 | 0 | 0 | 0 | 0 | 100 | 0 |
| TRACH3002605 | 0 | 0 | 0 | 0 | 0 | 52.334 | 0 |
| TRACH3002650 | 0 | 0 | 0 | 0 | 0 | 100 | 0 |
| TRACH3002752 | 0 | 0 | 0 | 0 | 0 | 11.442 | 0 |
| TRACH3002866 | 0 | 0 | 0 | 0 | 0 | 100 | 0 |
| TRACH3002871 | 0 | 0 | 0 | 0 | 0 | 100 | 0 |
| TRACH3002876 | 0 | 0 | 0 | 0 | 0 | 100 | 0 |
| TRACH3002890 | 0 | 0 | 0 | 0 | 0 | 100 | 0 |
| TRACH3003009 | 0 | 0 | 0 | 0 | 0 | 100 | 0 |
| TRACH3003037 | 0 | 0 | 0 | 0 | 0 | 35.900 | 0 |
| TRACH3003357 | 0 | 0 | 0 | 0 | 0 | 100 | 0 |
| TRACH3003379 | 0 | 0 | 0 | 0 | 0 | 54.819 | 0 |
| TRACH3003547 | 3.526 | 0 | 29.074 | 0 | 0 | 9.474 | 0 |
| TRACH3003586 | 0 | 0 | 0 | 0 | 0 | 100 | 0 |
| TRACH3003683 | 0 | 0 | 0 | 0 | 0 | 100 | 0 |
| TRACH3003780 | 0 | 0 | 0 | 0 | 0 | 100 | 0 |
| TRACH3003805 | 0 | 0 | 0 | 0 | 0 | 100 | 0 |
| TRACH3003832 | 0 | 0 | 0 | 0 | 0 | 13.601 | 0 |
| TRACH3003872 | 0 | 0 | 0 | 0 | 0 | 46.116 | 0 |
| TRACH3004068 | 0 | 0 | 0 | 0 | 31.044 | 1.596 | 0 |
| TRACH3004113 | 0 | 0 | 0 | 0 | 0 | 100 | 0 |
| TRACH3004288 | 0 | 0 | 0 | 0 | 0 | 100 | 0 |
| TRACH3004303 | 0 | 0 | 0 | 0 | 0 | 100 | 0 |
| TRACH3004412 | 0 | 0 | 0 | 0 | 0 | 100 | 0 |
| TRACH3004424 | 14.181 | 0 | 0 | 0 | 0 | 19.049 | 0 |
| TRACH3004456 | 0 | 0 | 0 | 0 | 0 | 100 | 0 |
| TRACH3004537 | 0 | 0 | 0 | 0 | 0 | 100 | 0 |
| TRACH3004596 | 0 | 0 | 0 | 0 | 0 | 100 | 0 |
| TRACH3004651 | 0 | 0 | 0 | 0 | 0 | 100 | 0 |
| TRACH3004652 | 0 | 0 | 0 | 0 | 0 | 100 | 0 |
| TRACH3004671 | 0 | 0 | 0 | 0 | 0 | 100 | 0 |
| TRACH3004721 | 2.424 | 0 | 0 | 0 | 0 | 3.256 | 0 |
| TRACH3004747 | 0 | 0 | 0 | 0 | 0 | 100 | 0 |
| TRACH3004760 | 0 | 0 | 0 | 0 | 0 | 100 | 0 |
| TRACH3004783 | 0 | 0 | 0 | 0 | 0 | 100 | 0 |
| TRACH3004786 | 0 | 0 | 0 | 0 | 0 | 100 | 0 |
| TRACH3004840 | 0 | 0 | 0 | 0 | 0 | 100 | 0 |
| TRACH3004931 | 0 | 0 | 0 | 0 | 0 | 100 | 0 |
| TRACH3004934 | 0 | 0 | 0 | 0 | 0 | 100 | 0 |
| TRACH3005072 | 0 | 0 | 0 | 0 | 0 | 100 | 0 |
| TRACH3005085 | 0 | 0 | 0 | 0 | 0 | 100 | 0 |
| TRACH3005102 | 0 | 0 | 0 | 0 | 0 | 100 | 0 |
| TRACH3005173 | 0 | 0 | 0 | 0 | 0 | 100 | 0 |
| TRACH3005191 | 0 | 0 | 0 | 0 | 0 | 100 | 0 |
| TRACH3005211 | 0 | 0 | 0 | 0 | 0 | 20.643 | 0 |
| TRACH3005274 | 0 | 0 | 0 | 0 | 0 | 100 | 0 |
| TRACH3005294 | 0 | 0 | 0 | 0 | 0 | 4.160 | 0 |
| TRACH3005479 | 0 | 0 | 0 | 0 | 0 | 63.148 | 0 |
| TRACH3005549 | 0 | 0 | 0 | 0 | 0 | 100 | 0 |
| TRACH3005699 | 0 | 0 | 0 | 0 | 0 | 53.752 | 0 |
| TRACH3005702 | 0 | 0 | 0 | 0 | 0 | 100 | 0 |
| TRACH3005808 | 0 | 0 | 0 | 0 | 0 | 100 | 0 |
| TRACH3005852 | 0 | 0 | 0 | 0 | 0 | 100 | 0 |
| TRACH3005896 | 0 | 0 | 0 | 0 | 0 | 100 | 0 |
| TRACH3006038 | 0 | 0 | 0 | 0 | 0 | 61.213 | 0 |
| TRACH3006149 | 0 | 0 | 0 | 0 | 0 | 100 | 0 |
| TRACH3006228 | 0 | 0 | 0 | 0 | 0 | 100 | 0 |
| TRACH3006264 | 0 | 0 | 0 | 0 | 0 | 100 | 0 |
| TRACH3006336 | 0 | 0 | 0 | 0 | 0 | 100 | 0 |
| TRACH3006379 | 0 | 0 | 0 | 0 | 0 | 100 | 0 |
| TRACH3006392 | 0 | 0 | 0 | 0 | 0 | 100 | 0 |
| TRACH3006395 | 0 | 0 | 0 | 0 | 0 | 100 | 0 |
| TRACH3006397 | 0 | 0 | 0 | 0 | 0 | 100 | 0 |
| TRACH3006412 | 0 | 0 | 0 | 0 | 0 | 61.213 | 0 |
| TRACH3006470 | 0 | 0 | 0 | 0 | 0 | 100 | 0 |
| TRACH3006589 | 0 | 0 | 0 | 0 | 0 | 100 | 0 |
| TRACH3006626 | 0 | 0 | 0 | 0 | 0 | 100 | 0 |
| TRACH3006699 | 0 | 0 | 0 | 0 | 0 | 100 | 0 |
| TRACH3006717 | 3.060 | 0 | 0 | 0 | 0 | 4.110 | 0 |
| TRACH3006800 | 0 | 0 | 0 | 0 | 0 | 65.258 | 0 |
| TRACH3006889 | 0 | 0 | 0 | 0 | 0 | 100 | 0 |
| TRACH3006985 | 0 | 0 | 0 | 0 | 0 | 100 | 0 |
| TRACH3007097 | 0 | 0 | 0 | 0 | 0 | 100 | 0 |
| TRACH3007268 | 0 | 0 | 0 | 0 | 0 | 100 | 0 |
| TRACH3007274 | 0 | 0 | 0 | 0 | 0 | 100 | 0 |
| TRACH3007277 | 0 | 0 | 0 | 0 | 0 | 100 | 0 |
| TRACH3007391 | 0 | 0 | 0 | 0 | 0 | 100 | 0 |
| TRACH3007479 | 0 | 0 | 0 | 0 | 0 | 26.502 | 0 |
| TRACH3007541 | 42.674 | 0 | 0 | 0 | 0 | 57.326 | 0 |
| TRACH3007595 | 0 | 0 | 0 | 0 | 0 | 100 | 0 |
| TRACH3007625 | 2.941 | 0 | 0 | 0 | 0 | 2.633 | 0 |
| TRACH3007627 | 0 | 0 | 0 | 0 | 0 | 100 | 0 |
| TRACH3007689 | 0 | 0 | 0 | 0 | 0 | 52.833 | 0 |
| TRACH3007766 | 0 | 0 | 0 | 0 | 0 | 100 | 0 |
| TRACH3007866 | 0 | 0 | 0 | 0 | 0 | 100 | 0 |
| TRACH3007995 | 0 | 0 | 0 | 0 | 0 | 61.213 | 0 |
| TRACH3008042 | 0 | 0 | 0 | 0 | 0 | 100 | 0 |
| TRACH3008061 | 0 | 0 | 0 | 0 | 0 | 100 | 0 |
| TRACH3008093 | 2.042 | 0 | 0 | 0 | 0 | 2.744 | 0 |
| TRACH3008201 | 0 | 0 | 0 | 0 | 0 | 52.833 | 0 |
| TRACH3008508 | 15.975 | 0 | 0 | 0 | 0 | 21.459 | 0 |
| TRACH3008535 | 0 | 0 | 0 | 0 | 0 | 31.730 | 0 |
| TRACH3008629 | 0 | 0 | 0 | 0 | 0 | 97.020 | 0 |
| TRACH3008632 | 0 | 0 | 0 | 0 | 0 | 9.465 | 0 |
| TRACH3008688 | 0 | 0 | 0 | 0 | 0 | 100 | 0 |
| TRACH3008697 | 0 | 0 | 0 | 0 | 0 | 100 | 0 |
| TRACH3008713 | 0 | 0 | 0 | 0 | 0 | 31.730 | 0 |
| TRACH3008887 | 0 | 0 | 0 | 0 | 0 | 100 | 0 |
| TRACH3008902 | 0 | 0 | 0 | 0 | 0 | 100 | 0 |
| TRACH3008990 | 0 | 0 | 0 | 0 | 0 | 100 | 0 |
| TRACH3009001 | 0 | 0 | 0 | 0 | 0 | 52.489 | 0 |
| TRACH3009008 | 0 | 0 | 0 | 0 | 0 | 16.463 | 0 |
| TRACH3009059 | 0 | 0 | 0 | 0 | 0 | 100 | 0 |
| TRACH3009061 | 0 | 0 | 0 | 0 | 0 | 100 | 0 |
| TRACH3009148 | 0 | 0 | 0 | 0 | 0 | 20.094 | 0 |
| TRACH3009455 | 0 | 0 | 0 | 0 | 0 | 52.833 | 0 |
| TRACH3009701 | 0 | 0 | 0 | 0 | 0 | 100 | 0 |
| TRACH3009715 | 0 | 0 | 0 | 0 | 0 | 100 | 0 |
| TRACH3009894 | 0 | 0 | 0 | 0 | 0 | 100 | 0 |
| TRACH3009956 | 0 | 0 | 0 | 0 | 0 | 44.106 | 0 |
| TRACH3010079 | 0 | 0 | 0 | 0 | 0 | 27.054 | 0 |
| TRACH3010167 | 0 | 0 | 0 | 0 | 0 | 100 | 0 |
| TRACH3010331 | 0 | 0 | 0 | 0 | 0 | 100 | 0 |
| TRACH3010342 | 0 | 0 | 0 | 0 | 0 | 100 | 0 |
| TRACH3010382 | 0 | 0 | 0 | 0 | 0 | 100 | 0 |
| TRACH3010757 | 0 | 0 | 0 | 0 | 0 | 100 | 0 |
| TRACH3010901 | 0 | 0 | 0 | 0 | 0 | 100 | 0 |
| TRACH3011004 | 0 | 0 | 0 | 0 | 0 | 100 | 0 |
| TRACH3011082 | 0 | 0 | 0 | 0 | 0 | 100 | 0 |
| TRACH3011184 | 0 | 0 | 0 | 0 | 0 | 100 | 0 |
| TRACH3011282 | 0 | 0 | 0 | 0 | 0 | 100 | 0 |
| TRACH3011313 | 0 | 0 | 0 | 0 | 0 | 100 | 0 |
| TRACH3011454 | 0 | 0 | 0 | 0 | 0 | 100 | 0 |
| TRACH3011485 | 0 | 0 | 0 | 0 | 0 | 100 | 0 |
| TRACH3011503 | 0 | 0 | 0 | 0 | 0 | 100 | 0 |
| TRACH3011538 | 0 | 0 | 0 | 0 | 0 | 100 | 0 |
| TRACH3011542 | 0 | 0 | 0 | 0 | 0 | 100 | 0 |
| TRACH3012106 | 0 | 0 | 0 | 0 | 0 | 100 | 0 |
| TRACH3012161 | 0 | 0 | 0 | 0 | 0 | 100 | 0 |
| TRACH3012232 | 0 | 0 | 0 | 0 | 0 | 100 | 0 |
| TRACH3012261 | 0 | 0 | 0 | 0 | 0 | 100 | 0 |
| TRACH3012445 | 0 | 0 | 0 | 0 | 0 | 100 | 0 |
| TRACH3012460 | 0 | 0 | 0 | 0 | 0 | 100 | 0 |
| TRACH3012659 | 0 | 0 | 0 | 0 | 0 | 100 | 0 |
| TRACH3012718 | 0 | 0 | 0 | 0 | 0 | 100 | 0 |
| TRACH3012864 | 0 | 0 | 0 | 0 | 0 | 100 | 0 |
| TRACH3012891 | 0 | 0 | 0 | 0 | 0 | 100 | 0 |
| TRACH3012942 | 0 | 0 | 0 | 0 | 0 | 100 | 0 |
| TRACH3013043 | 0 | 0 | 0 | 0 | 0 | 46.144 | 0 |
| TRACH3013072 | 42.674 | 0 | 0 | 0 | 0 | 57.326 | 0 |
| TRACH3013426 | 0 | 0 | 0 | 0 | 0 | 100 | 0 |
| TRACH3013454 | 0 | 0 | 0 | 0 | 0 | 100 | 0 |
| TRACH3013558 | 0 | 0 | 0 | 0 | 0 | 61.213 | 0 |
| TRACH3013684 | 0 | 0 | 0 | 0 | 0 | 100 | 0 |
| TRACH3013693 | 0 | 0 | 0 | 0 | 0 | 100 | 0 |
| TRACH3013700 | 0 | 0 | 0 | 0 | 0 | 100 | 0 |
| TRACH3013766 | 0 | 0 | 0 | 0 | 0 | 100 | 0 |
| TRACH3013900 | 0 | 0 | 0 | 0 | 0 | 100 | 0 |
| TRACH3013926 | 0 | 0 | 0 | 0 | 0 | 100 | 0 |
| TRACH3014063 | 0 | 0 | 0 | 0 | 0 | 100 | 0 |
| TRACH3014183 | 0 | 0 | 0 | 0 | 0 | 100 | 0 |
| TRACH3014215 | 0 | 0 | 0 | 0 | 0 | 100 | 0 |
| TRACH3014316 | 0 | 0 | 0 | 0 | 0 | 47.415 | 0 |
| TRACH3014415 | 0 | 0 | 0 | 0 | 0 | 100 | 0 |
| TRACH3014580 | 0 | 0 | 0 | 0 | 0 | 100 | 0 |
| TRACH3014641 | 0 | 0 | 0 | 0 | 0 | 100 | 0 |
| TRACH3014885 | 0 | 0 | 0 | 0 | 0 | 100 | 0 |
| TRACH3015136 | 0 | 0 | 0 | 0 | 0 | 100 | 0 |
| TRACH3015336 | 0 | 0 | 0 | 0 | 0 | 100 | 0 |
| TRACH3015346 | 0 | 0 | 0 | 0 | 0 | 100 | 0 |
| TRACH3015354 | 0 | 0 | 0 | 0 | 0 | 100 | 0 |
| TRACH3015467 | 0 | 0 | 0 | 0 | 0 | 100 | 0 |
| TRACH3015626 | 0 | 0 | 0 | 0 | 0 | 100 | 0 |
| TRACH3015951 | 0 | 0 | 0 | 0 | 0 | 100 | 0 |
| TRACH3016051 | 0 | 0 | 0 | 0 | 0 | 100 | 0 |
| TRACH3016120 | 0 | 0 | 0 | 0 | 0 | 100 | 0 |
| TRACH3016264 | 0 | 0 | 0 | 0 | 0 | 100 | 0 |
| TRACH3016277 | 0 | 0 | 0 | 0 | 0 | 100 | 0 |
| TRACH3016368 | 0 | 0 | 0 | 0 | 0 | 100 | 0 |
| TRACH3016455 | 0 | 0 | 0 | 0 | 0 | 100 | 0 |
| TRACH3016614 | 0 | 0 | 0 | 0 | 0 | 100 | 0 |
| TRACH3016805 | 11.263 | 0 | 0 | 0 | 0 | 5.043 | 0 |
| TRACH3016992 | 0 | 0 | 0 | 0 | 0 | 53.323 | 0 |
| TRACH3017171 | 0 | 0 | 0 | 0 | 0 | 100 | 0 |
| TRACH3017409 | 0 | 0 | 0 | 0 | 0 | 100 | 0 |
| TRACH3017934 | 0 | 0 | 0 | 0 | 0 | 100 | 0 |
| TRACH3018108 | 0 | 0 | 0 | 0 | 0 | 100 | 0 |
| TRACH3018191 | 0 | 0 | 0 | 0 | 0 | 100 | 0 |
| TRACH3018240 | 0 | 0 | 0 | 0 | 0 | 100 | 0 |
| TRACH3018261 | 0 | 0 | 0 | 0 | 0 | 100 | 0 |
| TRACH3018519 | 0 | 0 | 0 | 0 | 0 | 100 | 0 |
| TRACH3018524 | 0 | 0 | 0 | 0 | 0 | 100 | 0 |
| TRACH3018606 | 0 | 0 | 0 | 0 | 0 | 100 | 0 |
| TRACH3018783 | 0 | 0 | 0 | 0 | 0 | 100 | 0 |
| TRACH3018907 | 0 | 0 | 0 | 0 | 0 | 100 | 0 |
| TRACH3018933 | 0 | 0 | 0 | 0 | 0 | 100 | 0 |
| TRACH3018943 | 0 | 0 | 0 | 0 | 0 | 100 | 0 |
| TRACH3019018 | 0 | 0 | 0 | 0 | 0 | 100 | 0 |
| TRACH3019058 | 0 | 0 | 0 | 0 | 0 | 100 | 0 |
| TRACH3019142 | 0 | 0 | 0 | 0 | 0 | 100 | 0 |
| TRACH3019290 | 23.065 | 0 | 0 | 0 | 0 | 30.984 | 0 |
| TRACH3019347 | 0 | 0 | 0 | 0 | 0 | 100 | 0 |
| TRACH3019370 | 0 | 0 | 0 | 0 | 0 | 100 | 0 |
| TRACH3019447 | 0 | 0 | 0 | 0 | 0 | 100 | 0 |
| TRACH3019598 | 0 | 0 | 0 | 0 | 0 | 100 | 0 |
| TRACH3019621 | 0 | 0 | 0 | 0 | 0 | 100 | 0 |
| TRACH3019662 | 0 | 0 | 0 | 0 | 0 | 100 | 0 |
| TRACH3019807 | 0 | 0 | 0 | 0 | 0 | 100 | 0 |
| TRACH3019924 | 0 | 0 | 0 | 0 | 0 | 100 | 0 |
| TRACH3019977 | 0 | 0 | 0 | 0 | 0 | 100 | 0 |
| TRACH3020137 | 0 | 0 | 0 | 0 | 0 | 100 | 0 |
| TRACH3020410 | 0 | 0 | 0 | 0 | 0 | 100 | 0 |
| TRACH3020563 | 0 | 0 | 0 | 0 | 0 | 100 | 0 |
| TRACH3020605 | 0 | 0 | 0 | 0 | 0 | 100 | 0 |
| TRACH3020750 | 0 | 0 | 0 | 0 | 0 | 100 | 0 |
| TRACH3020769 | 0 | 0 | 0 | 0 | 0 | 100 | 0 |
| TRACH3020928 | 0 | 0 | 0 | 0 | 0 | 100 | 0 |
| TRACH3020930 | 0 | 0 | 0 | 0 | 0 | 100 | 0 |
| TRACH3021023 | 0 | 0 | 0 | 0 | 0 | 100 | 0 |
| TRACH3021066 | 0 | 0 | 0 | 0 | 0 | 100 | 0 |
| TRACH3021212 | 0 | 0 | 0 | 0 | 0 | 100 | 0 |
| TRACH3021335 | 0 | 0 | 0 | 0 | 0 | 100 | 0 |
| TRACH3021373 | 0 | 0 | 0 | 0 | 0 | 100 | 0 |
| TRACH3021544 | 0 | 0 | 0 | 0 | 0 | 47.415 | 0 |
| TRACH3021778 | 0 | 0 | 0 | 0 | 0 | 100 | 0 |
| TRACH3021834 | 0 | 0 | 0 | 0 | 0 | 100 | 0 |
| TRACH3021883 | 0 | 0 | 0 | 0 | 0 | 100 | 0 |
| TRACH3022170 | 0 | 0 | 0 | 0 | 0 | 100 | 0 |
| TRACH3022198 | 20.791 | 0 | 0 | 0 | 0 | 9.310 | 0 |
| TRACH3022296 | 0 | 0 | 0 | 0 | 0 | 100 | 0 |
| TRACH3022482 | 0 | 0 | 0 | 0 | 0 | 100 | 0 |
| TRACH3022732 | 0 | 0 | 0 | 0 | 0 | 100 | 0 |
| TRACH3022758 | 0 | 0 | 0 | 0 | 0 | 100 | 0 |
| TRACH3022875 | 0 | 0 | 0 | 0 | 0 | 100 | 0 |
| TRACH3022910 | 0 | 0 | 0 | 0 | 0 | 100 | 0 |
| TRACH3022960 | 0 | 0 | 0 | 0 | 0 | 100 | 0 |
| TRACH3023063 | 0 | 0 | 0 | 0 | 0 | 100 | 0 |
| TRACH3023203 | 0 | 0 | 0 | 0 | 0 | 100 | 0 |
| TRACH3023242 | 0 | 0 | 0 | 0 | 0 | 100 | 0 |
| TRACH3023373 | 0 | 0 | 0 | 0 | 0 | 100 | 0 |
| TRACH3023516 | 0 | 0 | 0 | 0 | 0 | 100 | 0 |
| TRACH3023752 | 0 | 0 | 0 | 0 | 0 | 100 | 0 |
| TRACH3023945 | 0 | 0 | 0 | 0 | 0 | 100 | 0 |
| TRACH3023960 | 0 | 0 | 0 | 0 | 0 | 100 | 0 |
| TRACH3024020 | 0 | 0 | 0 | 0 | 0 | 100 | 0 |
| TRACH3024081 | 0 | 0 | 0 | 0 | 0 | 100 | 0 |
| TRACH3024342 | 0 | 0 | 0 | 0 | 0 | 52.833 | 0 |
| TRACH3024423 | 0 | 0 | 0 | 0 | 0 | 100 | 0 |
| TRACH3024428 | 0 | 0 | 0 | 0 | 0 | 100 | 0 |
| TRACH3024512 | 0 | 0 | 0 | 0 | 0 | 100 | 0 |
| TRACH3024671 | 0 | 0 | 0 | 0 | 0 | 52.833 | 0 |
| TRACH3024823 | 0 | 0 | 0 | 0 | 0 | 100 | 0 |
| TRACH3025302 | 0 | 0 | 0 | 0 | 0 | 100 | 0 |
| TRACH3025316 | 0 | 0 | 0 | 0 | 0 | 68.709 | 0 |
| TRACH3025346 | 0 | 0 | 0 | 0 | 0 | 100 | 0 |
| TRACH3025520 | 0 | 0 | 0 | 0 | 0 | 100 | 0 |
| TRACH3026148 | 0 | 0 | 0 | 0 | 0 | 100 | 0 |
| TRACH3026283 | 0 | 0 | 0 | 0 | 0 | 100 | 0 |
| TRACH3026299 | 0 | 0 | 0 | 0 | 0 | 100 | 0 |
| TRACH3026303 | 0 | 0 | 0 | 0 | 0 | 31.391 | 0 |
| TRACH3026542 | 0 | 0 | 0 | 0 | 0 | 100 | 0 |
| TRACH3026650 | 0 | 0 | 0 | 0 | 0 | 100 | 0 |
| TRACH3026676 | 9.699 | 0 | 0 | 0 | 0 | 39.086 | 0 |
| TRACH3026744 | 0 | 0 | 0 | 0 | 0 | 100 | 0 |
| TRACH3026949 | 0 | 0 | 0 | 0 | 0 | 100 | 0 |
| TRACH3027229 | 0 | 0 | 0 | 0 | 0 | 6.069 | 0 |
| TRACH3027527 | 0 | 0 | 0 | 0 | 0 | 100 | 0 |
| TRACH3027681 | 0 | 0 | 0 | 0 | 0 | 100 | 0 |
| TRACH3027701 | 0 | 0 | 0 | 0 | 0 | 100 | 0 |
| TRACH3027885 | 0 | 0 | 0 | 0 | 0 | 100 | 0 |
| TRACH3028031 | 0 | 0 | 0 | 0 | 0 | 100 | 0 |
| TRACH3028164 | 0 | 0 | 0 | 0 | 0 | 100 | 0 |
| TRACH3028180 | 0 | 0 | 0 | 0 | 0 | 100 | 0 |
| TRACH3028195 | 0 | 0 | 0 | 0 | 0 | 100 | 0 |
| TRACH3028441 | 0 | 0 | 0 | 0 | 0 | 100 | 0 |
| TRACH3028597 | 0 | 0 | 0 | 0 | 0 | 100 | 0 |
| TRACH3028678 | 0 | 0 | 0 | 0 | 0 | 100 | 0 |
| TRACH3028837 | 0 | 0 | 0 | 0 | 0 | 100 | 0 |
| TRACH3028847 | 0 | 0 | 0 | 0 | 0 | 100 | 0 |
| TRACH3028855 | 0 | 0 | 0 | 0 | 0 | 49.978 | 0 |
| TRACH3029139 | 0 | 0 | 0 | 0 | 0 | 100 | 0 |
| TRACH3029329 | 0 | 0 | 0 | 0 | 0 | 100 | 0 |
| TRACH3029451 | 0 | 0 | 0 | 0 | 0 | 100 | 0 |
| TRACH3029462 | 0 | 0 | 0 | 0 | 0 | 100 | 0 |
| TRACH3029520 | 0 | 0 | 0 | 0 | 0 | 100 | 0 |
| TRACH3029592 | 0 | 0 | 0 | 0 | 0 | 100 | 0 |
| TRACH3029629 | 0 | 0 | 0 | 0 | 0 | 100 | 0 |
| TRACH3029665 | 0 | 0 | 0 | 0 | 0 | 100 | 0 |
| TRACH3029670 | 0 | 0 | 0 | 0 | 0 | 100 | 0 |
| TRACH3029800 | 0 | 0 | 0 | 0 | 0 | 100 | 0 |
| TRACH3030111 | 0 | 0 | 0 | 0 | 0 | 100 | 0 |
| TRACH3030130 | 0 | 0 | 0 | 0 | 0 | 100 | 0 |
| TRACH3030176 | 0 | 0 | 0 | 0 | 0 | 100 | 0 |
| TRACH3030252 | 0 | 0 | 0 | 0 | 0 | 100 | 0 |
| TRACH3030725 | 0 | 0 | 0 | 0 | 0 | 100 | 0 |
| TRACH3030855 | 0 | 0 | 0 | 0 | 0 | 100 | 0 |
| TRACH3030883 | 0 | 0 | 0 | 0 | 0 | 100 | 0 |
| TRACH3031316 | 0 | 0 | 0 | 0 | 0 | 100 | 0 |
| TRACH3031400 | 0 | 0 | 0 | 0 | 0 | 100 | 0 |
| TRACH3031463 | 0 | 0 | 0 | 0 | 0 | 100 | 0 |
| TRACH3031479 | 0 | 0 | 0 | 0 | 0 | 100 | 0 |
| TRACH3031518 | 0 | 0 | 0 | 0 | 0 | 100 | 0 |
| TRACH3031660 | 0 | 0 | 0 | 0 | 0 | 100 | 0 |
| TRACH3031678 | 0 | 0 | 0 | 0 | 0 | 100 | 0 |
| TRACH3031936 | 0 | 0 | 0 | 0 | 0 | 100 | 0 |
| TRACH3032044 | 0 | 0 | 0 | 0 | 0 | 100 | 0 |
| TRACH3032065 | 0 | 0 | 0 | 0 | 0 | 100 | 0 |
| TRACH3032150 | 0 | 0 | 0 | 0 | 0 | 100 | 0 |
| TRACH3032372 | 0 | 0 | 0 | 0 | 0 | 100 | 0 |
| TRACH3032480 | 0 | 0 | 0 | 0 | 0 | 100 | 0 |
| TRACH3032570 | 0 | 0 | 0 | 0 | 0 | 52.833 | 0 |
| TRACH3032755 | 0 | 0 | 0 | 0 | 0 | 100 | 0 |
| TRACH3032827 | 0 | 0 | 0 | 0 | 0 | 100 | 0 |
| TRACH3032873 | 0 | 0 | 0 | 0 | 0 | 100 | 0 |
| TRACH3033535 | 0 | 0 | 0 | 0 | 0 | 100 | 0 |
| TRACH3033680 | 0 | 0 | 0 | 0 | 0 | 100 | 0 |
| TRACH3033868 | 0 | 0 | 0 | 0 | 0 | 100 | 0 |
| TRACH3034145 | 0 | 0 | 0 | 0 | 0 | 100 | 0 |
| TRACH3034414 | 0 | 0 | 0 | 0 | 0 | 100 | 0 |
| TRACH3034488 | 0 | 0 | 0 | 0 | 0 | 100 | 0 |
| TRACH3034680 | 0 | 0 | 0 | 0 | 0 | 100 | 0 |
| TRACH3034731 | 0 | 0 | 0 | 0 | 0 | 100 | 0 |
| TRACH3034745 | 0 | 0 | 0 | 0 | 0 | 100 | 0 |
| TRACH3034762 | 0 | 0 | 0 | 0 | 0 | 100 | 0 |
| TRACH3034903 | 0 | 0 | 0 | 0 | 0 | 100 | 0 |
| TRACH3035187 | 0 | 0 | 0 | 0 | 0 | 100 | 0 |
| TRACH3035199 | 0 | 0 | 0 | 0 | 0 | 100 | 0 |
| TRACH3035235 | 0 | 0 | 0 | 0 | 0 | 100 | 0 |
| TRACH3035451 | 0 | 0 | 0 | 0 | 0 | 100 | 0 |
| TRACH3035482 | 0 | 0 | 0 | 0 | 0 | 100 | 0 |
| TRACH3035526 | 0 | 0 | 0 | 0 | 0 | 100 | 0 |
| TRACH3036004 | 0 | 0 | 0 | 0 | 0 | 100 | 0 |
| TRACH3036103 | 0 | 0 | 0 | 0 | 0 | 100 | 0 |
| TRACH3036193 | 0 | 0 | 0 | 0 | 0 | 100 | 0 |
| TRACH3036207 | 0 | 0 | 0 | 0 | 0 | 100 | 0 |
| TRACH3036278 | 0 | 0 | 0 | 0 | 0 | 100 | 0 |
| TRACH3036309 | 0 | 0 | 0 | 0 | 0 | 100 | 0 |
| TRACH3036456 | 0 | 0 | 0 | 0 | 0 | 100 | 0 |
| TRACH3036609 | 0 | 0 | 0 | 0 | 0 | 100 | 0 |
| TRACH3036638 | 0 | 0 | 0 | 0 | 0 | 100 | 0 |
| TRACH3036683 | 0 | 0 | 0 | 0 | 0 | 100 | 0 |
| TRACH3036750 | 0 | 0 | 0 | 0 | 0 | 100 | 0 |
| TRACH3036792 | 0 | 0 | 0 | 0 | 0 | 100 | 0 |
| TRACH3036843 | 0 | 0 | 0 | 0 | 0 | 100 | 0 |
| TRACH3036897 | 0 | 0 | 0 | 0 | 0 | 100 | 0 |
| TRACH3036932 | 0 | 0 | 0 | 0 | 0 | 16.420 | 0 |
| TRACH3036942 | 0 | 0 | 0 | 0 | 0 | 0.273 | 0 |
| TRACH3036997 | 0 | 0 | 0 | 0 | 0 | 100 | 0 |
| TRACH3037063 | 0 | 0 | 0 | 0 | 0 | 100 | 0 |
| TRACH3037067 | 1.340 | 0 | 0 | 0 | 0 | 3.601 | 0 |
| TRACH3037267 | 0 | 0 | 0 | 0 | 0 | 100 | 0 |
| TRACH3037288 | 0 | 0 | 0 | 0 | 0 | 100 | 0 |
| TRACH3037505 | 0 | 0 | 0 | 0 | 0 | 100 | 0 |
| TRACH3037573 | 0 | 0 | 0 | 0 | 0 | 100 | 0 |
| TRACH3037696 | 0 | 0 | 0 | 0 | 0 | 100 | 0 |
| TRACH3037897 | 0 | 0 | 0 | 0 | 0 | 100 | 0 |
| TRACH3038086 | 0 | 0 | 0 | 0 | 0 | 100 | 0 |
| TRACH3038399 | 0 | 0 | 0 | 0 | 0 | 41.787 | 0 |
| TSTOM1000135 | 0 | 0 | 0 | 0 | 0 | 0 | 90.639 |
| TSTOM1000186 | 0 | 0 | 0 | 0 | 0 | 0 | 100 |
| TSTOM2000139 | 0 | 0 | 0 | 0 | 0 | 0 | 89.823 |
| TSTOM2000235 | 0 | 0 | 0 | 0 | 0 | 0 | 95.465 |
| TSTOM2000315 | 0 | 0 | 0 | 0 | 0 | 0 | 48.943 |
| TSTOM2000442 | 0 | 0 | 0 | 0 | 0 | 0 | 100 |
| TSTOM2000553 | 0 | 0 | 0 | 0 | 0 | 24.547 | 36.203 |
| TSTOM2000569 | 0 | 0 | 0 | 0 | 0 | 0 | 100 |
| TSTOM2000588 | 0 | 0 | 0 | 0 | 0 | 0 | 100 |
| TSTOM2001195 | 0 | 0 | 0 | 0 | 0 | 0 | 100 |
| TSTOM2001274 | 0 | 0 | 0 | 0 | 0 | 0 | 100 |
| TSTOM2001571 | 0 | 0 | 0 | 0 | 0 | 0 | 100 |
| TSTOM2001996 | 0 | 0 | 0 | 0 | 0 | 0 | 100 |
| TSTOM2002265 | 0 | 0 | 0 | 0 | 0 | 0 | 100 |
| TSTOM2002505 | 0 | 0 | 0 | 0 | 0 | 0 | 100 |
| TSTOM2002561 | 0 | 0 | 0 | 0 | 0 | 0 | 100 |
| TSTOM2002611 | 0 | 0 | 0 | 0 | 0 | 0 | 100 |
| TSTOM2002672 | 0 | 0 | 0 | 0 | 0 | 0 | 100 |
| TSTOM2002682 | 0 | 0 | 0 | 0 | 0 | 0 | 100 |
| TUTER1000122 | 2.722 | 0 | 0 | 0 | 0 | 10.968 | 0 |
| TUTER2000904 | 0 | 0 | 0 | 0 | 0 | 3.127 | 0 |
| TUTER2002323 | 2.716 | 0 | 22.391 | 0 | 0 | 0 | 0 |
| UMVEN1000143 | 0 | 0 | 0 | 0 | 17.888 | 0 | 0 |
| UMVEN2000121 | 0 | 0 | 0 | 0 | 0 | 1.161 | 0 |
| UMVEN2000133 | 0.977 | 0 | 0 | 0 | 0 | 0.657 | 0 |
| UMVEN2000152 | 1.314 | 0 | 0 | 0 | 0 | 0 | 0 |
| UMVEN2000354 | 0 | 3.049 | 0 | 0 | 0 | 0 | 0 |
| UMVEN2000453 | 8.708 | 0 | 0 | 0 | 0 | 0 | 0 |
| UTERU1000008 | 41.147 | 0 | 0 | 0 | 0 | 0 | 0 |
| UTERU1000106 | 4.805 | 0 | 39.618 | 0 | 0 | 0 | 0 |
| UTERU1000182 | 0.676 | 0 | 0 | 0 | 0 | 1.817 | 0 |
| UTERU1000384 | 0 | 0 | 0 | 0 | 0 | 15.967 | 0 |
| UTERU2000095 | 0.690 | 1.532 | 0 | 0 | 0 | 1.854 | 0 |
| UTERU2000260 | 52.081 | 0 | 0 | 0 | 0 | 0 | 0 |
| UTERU2000263 | 0 | 0 | 0 | 0 | 0 | 1.722 | 11.004 |
| UTERU2000424 | 1.416 | 3.144 | 0 | 0 | 18.499 | 0 | 0 |
| UTERU2000537 | 4.278 | 0 | 0 | 0 | 0 | 0 | 0 |
| UTERU2001607 | 1.230 | 0 | 0 | 0 | 0 | 6.609 | 0 |
| UTERU2002294 | 0 | 0 | 12.480 | 0 | 0 | 0 | 0 |
| UTERU2002410 | 0.979 | 2.173 | 4.037 | 0 | 0 | 1.315 | 0 |
| UTERU2002662 | 1.484 | 0 | 0 | 0 | 0 | 1.993 | 0 |
| UTERU2003704 | 0 | 0 | 0 | 0 | 0 | 19.280 | 0 |
| UTERU2004037 | 0 | 0 | 0 | 0 | 0 | 2.456 | 0 |
| UTERU2004197 | 3.353 | 0 | 0 | 0 | 0 | 0 | 0 |
| UTERU2004807 | 2.513 | 0 | 0 | 0 | 0 | 3.376 | 0 |
| UTERU2004861 | 19.728 | 0 | 0 | 0 | 0 | 0 | 0 |
| UTERU2005004 | 58.303 | 0 | 0 | 0 | 0 | 0 | 0 |
| UTERU2005074 | 28.406 | 0 | 0 | 0 | 0 | 0 | 0 |
| UTERU2005292 | 2.596 | 0 | 0 | 0 | 0 | 0 | 0 |
| UTERU2005354 | 5.472 | 8.097 | 0 | 0 | 0 | 0 | 0 |
| UTERU2005449 | 27.302 | 0 | 0 | 0 | 0 | 36.676 | 0 |
| UTERU2005593 | 0.936 | 12.464 | 15.433 | 0 | 0 | 7.543 | 0 |
| UTERU2005601 | 0 | 0 | 0 | 0 | 0 | 88.511 | 0 |
| UTERU2005621 | 1.880 | 0 | 0 | 0 | 0 | 2.525 | 0 |
| UTERU2006115 | 6.535 | 0 | 0 | 0 | 0 | 0 | 0 |
| UTERU2006593 | 0 | 0 | 10.151 | 0 | 0 | 28.116 | 0 |
| UTERU2008085 | 0.527 | 1.003 | 0 | 0 | 1.967 | 0.101 | 1.939 |
| UTERU2008302 | 1.114 | 0 | 0 | 0 | 0 | 1.496 | 0 |
| UTERU2008426 | 0 | 0 | 0 | 0 | 0 | 49.324 | 0 |
| UTERU2009335 | 0 | 0 | 0 | 0 | 0 | 3.397 | 0 |
| UTERU2009435 | 4.459 | 0 | 0 | 0 | 0 | 11.980 | 0 |
| UTERU2009972 | 2.954 | 0 | 0 | 0 | 0 | 7.937 | 0 |
| UTERU2010226 | 19.733 | 0 | 0 | 0 | 0 | 8.836 | 0 |
| UTERU2010417 | 0.263 | 0 | 0 | 0 | 0 | 2.825 | 6.771 |
| UTERU2011220 | 1.632 | 0 | 0 | 0 | 0 | 0 | 0 |
| UTERU2011287 | 27.368 | 0 | 0 | 0 | 0 | 0 | 0 |
| UTERU2011621 | 16.560 | 0 | 0 | 0 | 0 | 0 | 0 |
| UTERU2011741 | 9.823 | 0 | 0 | 0 | 0 | 0 | 0 |
| UTERU2011906 | 11.373 | 0 | 0 | 0 | 0 | 15.277 | 0 |
| UTERU2012230 | 0 | 0 | 0 | 0 | 0 | 1.971 | 0 |
| UTERU2012286 | 2.113 | 0 | 0 | 0 | 0 | 2.838 | 0 |
| UTERU2012688 | 0 | 0 | 0 | 0 | 0 | 48.432 | 0 |
| UTERU2012741 | 15.010 | 0 | 0 | 0 | 0 | 0 | 0 |
| UTERU2012767 | 0 | 0 | 36.610 | 0 | 0 | 5.965 | 0 |
| UTERU2015653 | 0 | 0 | 0 | 0 | 0 | 1.307 | 0 |
| UTERU2015830 | 41.147 | 0 | 0 | 0 | 0 | 0 | 0 |
| UTERU2016799 | 0 | 0 | 0 | 0 | 0 | 13.028 | 0 |
| UTERU2016822 | 0 | 0 | 0 | 0 | 0 | 15.760 | 0 |
| UTERU2018884 | 1.292 | 0 | 0 | 0 | 0 | 3.471 | 0 |
| UTERU2019005 | 0 | 0 | 0 | 0 | 0 | 7.234 | 0 |
| UTERU2019096 | 3.842 | 0 | 0 | 0 | 0 | 2.580 | 0 |
| UTERU2023175 | 2.483 | 0 | 0 | 0 | 0 | 0 | 0 |
| UTERU2023550 | 0 | 31.001 | 0 | 0 | 0 | 0 | 0 |
| UTERU2023651 | 41.897 | 0 | 0 | 0 | 0 | 28.140 | 0 |
| UTERU2024656 | 0 | 0 | 0 | 0 | 0 | 48.432 | 0 |
| UTERU2024969 | 13.389 | 0 | 0 | 0 | 0 | 3.597 | 0 |
| UTERU2028734 | 0 | 0 | 0 | 0 | 0 | 24.591 | 0 |
| UTERU2031084 | 36.254 | 0 | 0 | 0 | 0 | 0 | 0 |
| UTERU2034147 | 41.147 | 0 | 0 | 0 | 0 | 0 | 0 |
| UTERU2035114 | 0 | 0 | 0 | 0 | 0 | 48.432 | 0 |
| UTERU2036347 | 26.499 | 0 | 0 | 0 | 0 | 35.597 | 0 |
| UTERU3000298 | 3.858 | 0 | 0 | 0 | 0 | 20.730 | 0 |
| UTERU3000645 | 0 | 0 | 0 | 0 | 0 | 65.258 | 0 |
| UTERU3000738 | 58.303 | 0 | 0 | 0 | 0 | 0 | 0 |
| UTERU3001542 | 41.147 | 0 | 0 | 0 | 0 | 0 | 0 |
| UTERU3002218 | 0 | 0 | 0 | 0 | 0 | 48.432 | 0 |
| UTERU3002786 | 0 | 0 | 0 | 0 | 0 | 48.432 | 0 |
| UTERU3003135 | 27.368 | 0 | 0 | 0 | 0 | 0 | 0 |
| UTERU3003660 | 0 | 0 | 0 | 0 | 0 | 8.247 | 0 |
| UTERU3005049 | 41.147 | 0 | 0 | 0 | 0 | 0 | 0 |
| UTERU3005422 | 53.061 | 0 | 0 | 0 | 0 | 0 | 0 |
| UTERU3007021 | 0 | 0 | 0 | 0 | 0 | 65.258 | 0 |
| UTERU3007104 | 0 | 0 | 0 | 0 | 0 | 48.432 | 0 |
| UTERU3010409 | 0 | 0 | 0 | 0 | 0 | 7.754 | 0 |
| UTERU3011579 | 0 | 0 | 0 | 0 | 0 | 65.258 | 0 |
| UTERU3013302 | 41.147 | 0 | 0 | 0 | 0 | 0 | 0 |
| UTERU3018172 | 26.594 | 0 | 0 | 0 | 0 | 0 | 0 |
| UTERU3018711 | 0 | 0 | 0 | 0 | 0 | 13.534 | 0 |

Genes whose expression levels were negligibly low in each of the above-described libraries are shown with their clone names below.

3NB691000018, 3NB691000085, 3NB691000113, 3NB691000129, 3NB691000191, 3NB692000012, 3NB692000029, 3NB692000102, 3NB692000154, 3NB692000276, 3NB692000305, 3NB692000429, 3NB692000484, 3NB692000529, 3NB692000973, 3NB692001002, 3NB692001034, 3NB692001040, 3NB692001123, 3NB692001267, 3NB692001339, 3NB692001349, 3NB692001366, 3NB692001408, 3NB692001433, 3NB692001459, 3NB692001471, 3NB692001496, 3NB692001501, 3NB692001507, 3NB692001511, 3NB692001519, 3NB692001528, 3NB692001548, 3NB692001557, 3NB692001637, 3NB692001719, 3NB692001861, 3NB692002365, 3NB692002685, 3NB692002806, 3NB692003538, 3NB692004045, 3NB692004724, 3NB692005439, 3NB692006952, 3NB692008178, 3NB692008729, ACTVT2000380, ADIPS1000064,

ADIPS2000069, ADIPS2000088, ADIPS2000245, ADIPS2000425, ADRGL1000033, ADRGL1000038, ADRGL1000065, ADRGL1000067, ADRGL1000144, ADRGL1000147, ADRGL1000160, ADRGL1000165, ADRGL1000182, ADRGL2000006, ADRGL2000042, ADRGL2000064, ADRGL2000074, ADRGL2000085, ADRGL2000117, ADRGL2000142, ADRGL2000172, ADRGL2000248, ADRGL2000261, ADRGL2000323, ADRGL2000328, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756, ADRGL2001830, ADRGL2001836, ADRGL2001854, ADRGL2002013, ADRGL2002029, ADRGL2002191, ADRGL2002392, ADRGL2002679, ADRGL2002753, ADRGL2002857, ADRGL2003017,

ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006677, ADRGL2006732, ADRGL2006767, ADRGL2006817, ADRGL2006846, ADRGL2006886, ADRGL2007080, ADRGL2007125, ADRGL2007283, ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008966, ADRGL2009146, ADRGL2009324,

ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2012179, AHMSC1000138, ASTRO1000009, ASTRO1000018, ASTRO1000029, ASTRO1000096, ASTRO1000165, ASTRO2000011, ASTRO2000014, ASTRO2000095, ASTRO2000191, ASTRO2000372, ASTRO2000381, ASTRO2000403, ASTRO2000417, ASTRO2000482, ASTRO2000533, ASTRO2000653, ASTRO2000662, ASTRO2000725, ASTRO2001001, ASTRO2001008, ASTRO2001088, ASTRO2001107, ASTRO2001122, ASTRO2001227, ASTRO2001249, ASTRO2001480, ASTRO2002035, ASTRO2002459, ASTRO2002571, ASTRO2002632, ASTRO2002659, ASTRO2002693, ASTRO2002720, ASTRO2002733, ASTRO2002743, ASTRO2002842, ASTRO2003212, ASTRO2003574,

ASTRO2003581, ASTRO2003740, ASTRO2003925, ASTRO2003960, ASTRO2004032, ASTRO2004051, ASTRO2004177, ASTRO2004217, ASTRO2004297, ASTRO2004584, ASTRO2004751, ASTRO2004877, ASTRO2005008, ASTRO2005081, ASTRO2005096, ASTRO2005242, ASTRO2005273, ASTRO2005343, ASTRO2005413, ASTRO2005553, ASTRO2005557, ASTRO2005575, ASTRO2005726, ASTRO2005863, ASTRO2005896, ASTRO2005902, ASTRO2006356, ASTRO2006475, ASTRO2006732, ASTRO2006747, ASTRO2006952, ASTRO2007221, ASTRO2007377, ASTRO2007515, ASTRO2007578, ASTRO2007643, ASTRO2007666, ASTRO2007956, ASTRO2007962, ASTRO2008040, ASTRO2008239, ASTRO2008409, ASTRO2008425, ASTRO2008508, ASTRO2008777, ASTRO2008895, ASTRO2008960, ASTRO2008972, ASTRO2009177, ASTRO2009333,

ASTRO2009458, ASTRO2009526, ASTRO2009879, ASTRO2010072, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2013124, ASTRO2013802, ASTRO2014174, ASTRO2014211, ASTRO2014363, ASTRO2014392, ASTRO2014498, ASTRO2014576, ASTRO2014923, ASTRO2014961, ASTRO2015162, ASTRO2015185, ASTRO2015328, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000193, BGGI11000285,

BGGI12000022, BGGI12000067, BGGI12000161, BGGI12000544, BGGI12000616, BGGI12000684, BGGI12000693, BGGI12001097, BGGI12001241, BGGI12001247, BGGI12001714, BLADE1000176, BLADE2000181, BLADE2000256, BLADE2000340, BLADE2000389, BLADE2000463, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004345, BLADE2004389, BLADE2004462, BLADE2004628, BLADE2004670, BLADE2004722, BLADE2004849, BLADE2005036,

BLADE2005038, BLADE2005229, BLADE200545-9, BLADE2005792, BLADE2006043, BLADE2006415, BLADE2006607, BLADE2006826, BLADE2006849, BLADE2006947, BLADE2007033, BLADE2007043, BLADE2007722, BLADE2007735, BLADE2007744, BLADE2007799, BLADE2007923, BLADE2007935, BLADE2007958, BLADE2008281, BLADE2008454, BLADE2008479, BLADE2008539, BLADE2008809, BLADE2008995, BLADE2009452, BLADE2009479, BLADE2009593, BNGH41000104, BNGH41000118, BNGH41000169, BNGH41000177, BNGH41000198, BNGH41000216, BNGH42000130, BNGH42000132, BNGH42000405, BNGH42000532, BNGH42000675, BNGH42000791, BNGH42000815, BNGH42000994, BNGH42001247, BNGH42001406, BNGH42001576, BNGH42001724, BNGH42002244, BNGH42002387, BNGH42002487, BNGH42002489,

BNGH42003529, BNGH42003570, BNGH42003641, BNGH42004076, BNGH42004291, BNGH42004538, BNGH42004679, BNGH42005017, BNGH42005235, BNGH42005504, BNGH42006226, BNGH42006234, BNGH42007037, BNGH42007594, BNGH42007798, BNGH42008199, BNGH42008510, BNGH42008649, BNGH42008832, BNGH42008850, BNGH42008943, BNGH42009021, BNGH42009025, BRACE1000047, BRACE1000051, BRACE1000065, BRACE1000084, BRACE1000087, BRACE1000092, BRACE1000093, BRACE1000101, BRACE1000114, BRACE1000124, BRACE1000132, BRACE1000169, BRACE1000187, BRACE1000239, BRACE1000258, BRACE1000451, BRACE1000475, BRACE1000572, BRACE2000077, BRACE2000078, BRACE2000100, BRACE2000109, BRACE2000141, BRACE2000148, BRACE2000183, BRACE2000229, BRACE2000232,

BRACE2000256, BRACE2000266, BRACE2000267, BRACE2000280, BRACE2000307, BRACE2000331, BRACE2000351, BRACE2000368, BRACE2000392, BRACE2000441, BRACE2000489, BRACE2000505, BRACE2000526, BRACE2000531, BRACE2000545, BRACE2000553, BRACE2000577, BRACE2000640, BRACE2000650, BRACE2000677, BRACE2000698, BRACE2000718, BRACE2000733, BRACE2000743, BRACE2000750, BRACE2000815, BRACE2000885, BRACE2000905, BRACE2000936, BRACE2000947, BRACE2000965, BRACE2000969, BRACE2000988, BRACE2001017, BRACE2001070, BRACE2001094, BRACE2001107, BRACE2001151, BRACE2001191, BRACE2001197, BRACE2001236, BRACE2001239, BRACE2001269, BRACE2001312, BRACE2001330, BRACE2001340, BRACE2001352, BRACE2001353, BRACE2001374, BRACE2001375,

BRACE2001423, BRACE2001424, BRACE2001445, BRACE2001453, BRACE2001455, BRACE2001492, BRACE2001508, BRACE2001534, BRACE2001543, BRACE2001564, BRACE2001588, BRACE2001607, BRACE2001626, BRACE2001677, BRACE2001705, BRACE2001726, BRACE2001733, BRACE2001737, BRACE2001742, BRACE2001777, BRACE2001779, BRACE2001859, BRACE2001865, BRACE2001872, BRACE2001881, BRACE2001898, BRACE2001923, BRACE2001954, BRACE2001972, BRACE2001973, BRACE2002026, BRACE2002034, BRACE2002139, BRACE2002142, BRACE2002151, BRACE2002157, BRACE2002176, BRACE2002202, BRACE2002206, BRACE2002227, BRACE2002260, BRACE2002297, BRACE2002304, BRACE2002356, BRACE2002394, BRACE2002409, BRACE2002431, BRACE2002441, BRACE2002444, BRACE2002458,

BRACE2002467, BRACE2002468, BRACE2002478, BRACE2002495, BRACE2002582, BRACE2002585, BRACE2002589, BRACE2002590, BRACE2002606, BRACE2002613, BRACE2002614, BRACE2002617, BRACE2002623, BRACE2002635, BRACE2002676, BRACE2002682, BRACE2002695, BRACE2002707, BRACE2002736, BRACE2002752, BRACE2002762, BRACE2002772, BRACE2002792, BRACE2002796, BRACE2002803, BRACE2002807, BRACE2002834, BRACE2002849, BRACE2002857, BRACE2002861, BRACE2002884, BRACE2002896, BRACE2002948, BRACE2003078, BRACE2003097, BRACE2003110, BRACE2003168, BRACE2003199, BRACE2003210, BRACE2003217, BRACE2003269, BRACE2003285, BRACE2003319, BRACE2003398, BRACE2003420, BRACE2003428, BRACE2003431, BRACE2003512, BRACE2003516, BRACE2003539,

BRACE2003540, BRACE2003598, BRACE2003609, BRACE2003614, BRACE2003639, BRACE2003700, BRACE2003713, BRACE2003766, BRACE2003802, BRACE2003825, BRACE2003847, BRACE2003873, BRACE2003885, BRACE2003887, BRACE2003904, BRACE2003905, BRACE2003928, BRACE2003954, BRACE2003987, BRACE2005034, BRACE2005047, BRACE2005063, BRACE2005083, BRACE2005090, BRACE2005118, BRACE2005124, BRACE2005138, BRACE2005151, BRACE2005160, BRACE2005169, BRACE2005189, BRACE2005193, BRACE2005196, BRACE2005198, BRACE2005216, BRACE2005243, BRACE2005251, BRACE2005253, BRACE2005290, BRACE2005328, BRACE2005329, BRACE2005337, BRACE2005348, BRACE2005363, BRACE2005434, BRACE2005448, BRACE2005450, BRACE2005453, BRACE2005457, BRACE2005460,

BRACE2005489, BRACE2005518, BRACE2005556, BRACE2005562, BRACE2005624, BRACE2005642, BRACE2005661, BRACE2005667, BRACE2005681, BRACE2005719, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006072, BRACE2006075, BRACE2006089, BRACE2006122, BRACE2006137, BRACE2006143, BRACE2006167, BRACE2006174, BRACE2006240, BRACE2006245, BRACE2006274, BRACE2006281, BRACE2006298, BRACE2006344, BRACE2006354, BRACE2006380, BRACE2006393, BRACE2006413, BRACE2006417, BRACE2006453,

BRACE2006459, BRACE2006488, BRACE2006534, BRACE2006540, BRACE2006587, BRACE2006598, BRACE2006636, BRACE2006685, BRACE2006703, BRACE2006743, BRACE2006783, BRACE2006821, BRACE2006833, BRACE2006859, BRACE2006909, BRACE2006911, BRACE2006924, BRACE2006935, BRACE2006944, BRACE2006951, BRACE2006986, BRACE2007013, BRACE2007087, BRACE2007138, BRACE2007197, BRACE2007201, BRACE2007203, BRACE2007232, BRACE2007262, BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007522, BRACE2007527, BRACE2007538, BRACE2007563, BRACE2007577, BRACE2007621, BRACE2007640, BRACE2007641, BRACE2007646,

BRACE2007663, BRACE2007671, BRACE2007682, BRACE2007685, BRACE2007708, BRACE2007727, BRACE2007749, BRACE2007760, BRACE2007761, BRACE2007764, BRACE2007768, BRACE2007769, BRACE2007784, BRACE2007786, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008, BRACE2008015, BRACE2008021, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008361, BRACE2008401, BRACE2008410, BRACE2008420, BRACE2008443, BRACE2008473, BRACE2008480, BRACE2008481, BRACE2008488, BRACE2008495,

BRACE2008500, BRACE2008553, BRACE2008594, BRACE2008604, BRACE2008615, BRACE2008622, BRACE2008629, BRACE2008655, BRACE2008656, BRACE2008708, BRACE2008710, BRACE2008754, BRACE2008797, BRACE2008847, BRACE2008857, BRACE2008861, BRACE2008867, BRACE2008881, BRACE2008883, BRACE2008917, BRACE2008919, BRACE2008941, BRACE2008948, BRACE2008960, BRACE2008999, BRACE2009014, BRACE2009016, BRACE2009031, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009293, BRACE2009307, BRACE2009311, BRACE2009318, BRACE2009323, BRACE2009366, BRACE2009421,

BRACE2009428, BRACE2009434, BRACE2009437, BRACE2009517, BRACE2009533, BRACE2009566, BRACE2009591, BRACE2009654, BRACE2009721, BRACE2009732, BRACE2009754, BRACE2009828, BRACE2009886, BRACE2009907, BRACE2009957, BRACE2010170, BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010753, BRACE2010813, BRACE2010828, BRACE2010837, BRACE2010888, BRACE2010937, BRACE2010983, BRACE2011007, BRACE2011025, BRACE2011199, BRACE2011389, BRACE2011478, BRACE2011545, BRACE2011592, BRACE2011611, BRACE2011646, BRACE2011677, BRACE2011703, BRACE2011747, BRACE2011838,

BRACE2011892, BRACE2011904, BRACE2011947, BRACE2012061, BRACE2012062, BRACE2012185, BRACE2012222, BRACE2012232, BRACE2012291, BRACE2012510, BRACE2012625, BRACE2012653, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2014108, BRACE2014161, BRACE2014232, BRACE2014268, BRACE2014475, BRACE2014657, BRACE2014780, BRACE2014818, BRACE2014821, BRACE2014824, BRACE2014854, BRACE2015031, BRACE2015132, BRACE2015262, BRACE2015270, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315,

BRACE2016325, BRACE2016335, BRACE2016366, BRACE2016465, BRACE2016516, BRACE2016583, BRACE2016708, BRACE2016821, BRACE2016896, BRACE2017108, BRACE2017124, BRACE2017298, BRACE2017359, BRACE2017410, BRACE2017438, BRACE2017580, BRACE2017587, BRACE2017720, BRACE2017844, BRACE2017872, BRACE2017929, BRACE2017934, BRACE2017992, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019147, BRACE2019244, BRACE2019258, BRACE2019327, BRACE2019348, BRACE2019371, BRACE2019467, BRACE2019510, BRACE2019528, BRACE2019618, BRACE2019696, BRACE2019703, BRACE2019843, BRACE2019865, BRACE2019872, BRACE2020028,

BRACE2020077, BRACE2020097, BRACE2020157, BRACE2020278, BRACE2020358, BRACE2020467, BRACE2020584, BRACE2020652, BRACE2020709, BRACE2020742, BRACE2020756, BRACE2020863, BRACE2020982, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021721, BRACE2021932, BRACE2021936, BRACE2022030, BRACE2022158, BRACE2022196, BRACE2022270, BRACE2022328, BRACE2022333, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070, BRACE2023223, BRACE2023268, BRACE2023284, BRACE2023404,

BRACE2023451, BRACE2023540, BRACE2023633, BRACE2023718, BRACE2023744, BRACE2023800, BRACE2024222, BRACE2024610, BRACE2024627, BRACE2024781, BRACE2024826, BRACE2025018, BRACE2025316, BRACE2025333, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026836, BRACE2026920, BRACE2026971, BRACE2027018, BRACE2027221, BRACE2027258, BRACE2027312, BRACE2027382, BRACE2027389, BRACE2027408, BRACE2027767, BRACE2027770, BRACE2027879, BRACE2027896, BRACE2028171, BRACE2028392, BRACE2028410, BRACE2028636, BRACE2028741, BRACE2028956, BRACE2028970, BRACE2029112, BRACE2029396,

BRACE2029581, BRACE2029849, BRACE2030039, BRACE2030096, BRACE2030326, BRACE2030341, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622, BRACE2034870, BRACE2035003, BRACE2035120, BRACE2035124, BRACE2035191, BRACE2035278, BRACE2035441, BRACE2035485, BRACE2036005, BRACE2036096, BRACE2036743, BRACE2036830, BRACE2036834, BRACE2036900,

BRACE2037132, BRACE2037294, BRACE2037295, BRACE2037299, BRACE2037310, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039734, BRACE2039823, BRACE2039832, BRACE2039893, BRACE2039986, BRACE2040138, BRACE2040325, BRACE2040514, BRACE2040640, BRACE2040987, BRACE2041009, BRACE2041095, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142,

BRACE2043248, BRACE2043349, BRACE2043381, BRACE2043774, BRACE2043942, BRACE2044057, BRACE2044105, BRACE2044263, BRACE2044304, BRACE2044473, BRACE2044510, BRACE2044640, BRACE2044816, BRACE2044946, BRACE2044949, BRACE2045300, BRACE2045428, BRACE2045445, BRACE2045596, BRACE2045772, BRACE2045947, BRACE2045954, BRACE2046251, BRACE2046295, BRACE2046976, BRACE2047011, BRACE2047232, BRACE2047377, BRACE2047385, BRACE2047492, BRACE2047558, BRACE2047573, BRACE2047835, BRACE2047893, BRACE2047975, BRACE3000071, BRACE3000404, BRACE3000520, BRACE3000697, BRACE3000840, BRACE3001002, BRACE3001058, BRACE3001066, BRACE3001113, BRACE3001217, BRACE3001299, BRACE3001384, BRACE3001391, BRACE3001403, BRACE3001595,

BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003595, BRACE3003866, BRACE3003920, BRACE3004046, BRACE3004058, BRACE3004113, BRACE3004150, BRACE3004205, BRACE3004358, BRACE3004371, BRACE3004435, BRACE3004477, BRACE3004767, BRACE3004772, BRACE3004783, BRACE3004843, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005760, BRACE3005870, BRACE3005903, BRACE3006113, BRACE3006185, BRACE3006226, BRACE3006462, BRACE3006463, BRACE3006872,

BRACE3006917, BRACE3007058, BRACE3007084, BRACE3007085, BRACE3007322, BRACE3007472, BRACE3007480, BRACE3007559, BRACE3007625, BRACE3007642, BRACE3007649, BRACE3007767, BRACE3007869, BRACE3008066, BRACE3008092, BRACE3008137, BRACE3008255, BRACE3008337, BRACE3008384, BRACE3008720, BRACE3008772, BRACE3009044, BRACE3009075, BRACE3009090, BRACE3009127, BRACE3009197, BRACE3009237, BRACE3009265, BRACE3009297, BRACE3009377, BRACE3009392, BRACE3009539, BRACE3009574, BRACE3009701, BRACE3009708, BRACE3009724, BRACE3009993, BRACE3010076, BRACE3010079, BRACE3010086, BRACE3010221, BRACE3010315, BRACE3010397, BRACE3010416, BRACE3010435, BRACE3010702, BRACE3010862, BRACE3011271, BRACE3011447, BRACE3011505,

BRACE3011634, BRACE3011774, BRACE3012357, BRACE3012574, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714, BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3018083, BRACE3018308, BRACE3018963, BRACE3019055, BRACE3019071, BRACE3019084, BRACE3019570, BRACE3019611, BRACE3019817, BRACE3019828, BRACE3020151,

BRACE3020194, BRACE3020286, BRACE3020356, BRACE3020594, BRACE3020671, BRACE3020890, BRACE3021148, BRACE3021430, BRACE3021517, BRACE3021805, BRACE3022051, BRACE3022303, BRACE3022340, BRACE3022847, BRACE3023604, BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027480, BRACE3027931, BRACE3028214, BRACE3028241, BRACE3028360,

BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184, BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032771, BRACE3032980, BRACE3032983, BRACE3033525, BRACE3034159, BRACE3034183, BRACE3034389, BRACE3034964, BRACE3034993, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239,

BRACE3040504, BRACE3040644, BRACE3040856, BRACE3041059, BRACE3041162, BRACE3041827, BRACE3042046, BRACE3042210, BRACE3042409, BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2001279, BRALZ2001350, BRALZ2001445, BRALZ2001559, BRALZ2001743, BRALZ2001834, BRALZ2002174,

BRALZ2002418, BRALZ2002477, BRALZ2003119, BRALZ2003330, BRALZ2003453, BRALZ2003525, BRALZ2004397, BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009446, BRALZ2009486, BRALZ2010186, BRALZ2010842, BRALZ2011337, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085,

BRALZ2016498, BRALZ2017359, BRALZ2017531, BRALZ2017607, BRALZ2017620, BRALZ2018342, BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000411, BRAMY2000653, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604, BRAMY2002621,

BRAMY2002638, BRAMY2002739, BRAMY2002799, BRAMY2002853, BRAMY2002862, BRAMY2002956, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004387, BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005648, BRAMY2005662, BRAMY2005708, BRAMY2005711, BRAMY2006092, BRAMY2006118, BRAMY2006149, BRAMY2006397,

BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008977, BRAMY2008979, BRAMY2009034, BRAMY2009105, BRAMY2009123, BRAMY2009192, BRAMY2009344, BRAMY2009349, BRAMY2009394, BRAMY2009489, BRAMY2009508, BRAMY2009528, BRAMY2009557, BRAMY2009612, BRAMY2009693, BRAMY2009934, BRAMY2010068, BRAMY2010135, BRAMY2010145,

BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012119, BRAMY2012162, BRAMY2012163, BRAMY2012277, BRAMY2012340, BRAMY2012426, BRAMY2012497, BRAMY2012517, BRAMY2012555, BRAMY2012568, BRAMY2012691, BRAMY2012721, BRAMY2012731, BRAMY2012776, BRAMY2012880, BRAMY2012881, BRAMY2013200, BRAMY2013216, BRAMY2013405,

BRAMY2013414, BRAMY2013572, BRAMY2013621, BRAMY2013659, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2015251, BRAMY2015311, BRAMY2015420, BRAMY2015503, BRAMY2015516, BRAMY2015551, BRAMY2015559, BRAMY2015688, BRAMY2015705, BRAMY2015855, BRAMY2015890, BRAMY2015911, BRAMY2015925, BRAMY2016002, BRAMY2016020, BRAMY2016070, BRAMY2016251, BRAMY2016325, BRAMY2016327, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2018027, BRAMY2018106, BRAMY2018126, BRAMY2018272, BRAMY2018273, BRAMY2018279,

BRAMY2018295, BRAMY2018308, BRAMY2018396, BRAMY2018467, BRAMY2018635, BRAMY2019055, BRAMY2019111, BRAMY2019277, BRAMY2019300, BRAMY2019468, BRAMY2019509, BRAMY2019643, BRAMY2019710, BRAMY2019985, BRAMY2020050, BRAMY2020058, BRAMY2020106, BRAMY2020270, BRAMY2020354, BRAMY2020355, BRAMY2020445, BRAMY2020466, BRAMY2020520, BRAMY2020574, BRAMY2020634, BRAMY2020713, BRAMY2021040, BRAMY2021063, BRAMY2021098, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022263, BRAMY2022301, BRAMY2022320, BRAMY2022383, BRAMY2022454, BRAMY2022525, BRAMY2022532,

BRAMY2022723, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023482, BRAMY2023514, BRAMY2023719, BRAMY2023786, BRAMY2023852, BRAMY2023863, BRAMY2023939, BRAMY2024004, BRAMY2024073, BRAMY2024312, BRAMY2024374, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026091, BRAMY2026168,

BRAMY2026300, BRAMY2026347, BRAMY2026395, BRAMY2026405, BRAMY2026533, BRAMY2026538, BRAMY2026713, BRAMY2026778, BRAMY2026824, BRAMY2026899, BRAMY2026902, BRAMY2026904, BRAMY2026976, BRAMY2027073, BRAMY2027140, BRAMY2027242, BRAMY2027396, BRAMY2027451, BRAMY2027461, BRAMY2027467, BRAMY2027714, BRAMY2027717, BRAMY2027752, BRAMY2027782, BRAMY2028072, BRAMY2028183, BRAMY2028188, BRAMY2028269, BRAMY2028282, BRAMY2028472, BRAMY2028491, BRAMY2028565, BRAMY2028593, BRAMY2028783, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859,

BRAMY2030915, BRAMY2031317, BRAMY2031377, BRAMY2031442, BRAMY2032014, BRAMY2032231, BRAMY2032242, BRAMY2032290, BRAMY2032311, BRAMY2032317, BRAMY2032344, BRAMY2032589, BRAMY2032678, BRAMY2032777, BRAMY2032994, BRAMY2033003, BRAMY2033116, BRAMY2033240, BRAMY2033267, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035449, BRAMY2035545, BRAMY2035606, BRAMY2035718, BRAMY2035801, BRAMY2035869, BRAMY2035923, BRAMY2036079, BRAMY2036254, BRAMY2036266, BRAMY2036387, BRAMY2036396, BRAMY2036567, BRAMY2036615, BRAMY2036699, BRAMY2036913, BRAMY2036918, BRAMY2036968, BRAMY2037147,

BRAMY2037328, BRAMY2037609, BRAMY2037629, BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038887, BRAMY2038904, BRAMY2039287, BRAMY2039300, BRAMY2039314, BRAMY2039341, BRAMY2039630, BRAMY2039695, BRAMY2039728, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918,

BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002312, BRAMY3002329, BRAMY3002508, BRAMY3002620, BRAMY3002803, BRAMY3002805, BRAMY3002886, BRAMY3002991, BRAMY3003026, BRAMY3003109,

BRAMY3003566, BRAMY3003723, BRAMY3004126, BRAMY3004224, BRAMY3004672, BRAMY3004800, BRAMY3004900, BRAMY3004919, BRAMY3005091, BRAMY3005184, BRAMY3005656, BRAMY3005912, BRAMY3005932, BRAMY3006032, BRAMY3006297, BRAMY3006761, BRAMY3007078, BRAMY3007206, BRAMY3007350, BRAMY3007449, BRAMY3007471, BRAMY3007609, BRAMY3007723, BRAMY3007968, BRAMY3008088, BRAMY3008096, BRAMY3008335, BRAMY3008466, BRAMY3008505, BRAMY3008650, BRAMY3008937, BRAMY3009158, BRAMY3009207, BRAMY3009556, BRAMY3009782, BRAMY3009811, BRAMY3009904, BRAMY3010264, BRAMY3010321, BRAMY3010411, BRAMY3010603, BRAMY3010654, BRAMY4000095, BRAMY4000229, BRAMY4000277, BRAMY4000915, BRAMY4000962, BRAMY4001173, BRAMY4001200, BRAMY4001449,

BRAMY4001652, BRAMY4001913, BRAMY4002575, BRASW1000053, BRASW1000125, BRAWH1000002, BRAWH1000094, BRAWH1000116, BRAWH1000130, BRAWH1000157, BRAWH1000164, BRAWH1000168, BRAWH1000180, BRAWH1000369, BRAWH2000034, BRAWH2000048, BRAWH2000071, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256, BRAWH2000274, BRAWH2000323, BRAWH2000353, BRAWH2000370, BRAWH2000409, BRAWH2000443, BRAWH2000471, BRAWH2000476, BRAWH2000482, BRAWH2000500, BRAWH2000503, BRAWH2000522, BRAWH2000554, BRAWH2000595, BRAWH2000643, BRAWH2000651, BRAWH2000682, BRAWH2000752, BRAWH2000866, BRAWH2000892, BRAWH2000901, BRAWH2000926, BRAWH2000984,

BRAWH2001037, BRAWH2001052, BRAWH2001103, BRAWH2001137, BRAWH2001141, BRAWH2001159, BRAWH2001166, BRAWH2001203, BRAWH2001238, BRAWH2001255, BRAWH2001355, BRAWH2001378, BRAWH2001436, BRAWH2001438, BRAWH2001484, BRAWH2001492, BRAWH2001503, BRAWH2001519, BRAWH2001589, BRAWH2001662, BRAWH2001666, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002125, BRAWH2002191, BRAWH2002318, BRAWH2002333, BRAWH2002352, BRAWH2002383, BRAWH2002549, BRAWH2002560, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963,

BRAWH2002967, BRAWH2002976, BRAWH2003000, BRAWH2003025, BRAWH2003135, BRAWH2003307, BRAWH2003355, BRAWH2003366, BRAWH2003662, BRAWH2003832, BRAWH2003948, BRAWH2003964, BRAWH2004044, BRAWH2004068, BRAWH2004078, BRAWH2004580, BRAWH2004731, BRAWH2004779, BRAWH2004884, BRAWH2004923, BRAWH2005074, BRAWH2005315, BRAWH2005517, BRAWH2005524, BRAWH2005533, BRAWH2005812, BRAWH2005974, BRAWH2005998, BRAWH2006044, BRAWH2006083, BRAWH2006207, BRAWH2006301, BRAWH2006395, BRAWH2006405, BRAWH2006622, BRAWH2006760, BRAWH2006960, BRAWH2006989, BRAWH2006996, BRAWH2007069, BRAWH2007308, BRAWH2007406, BRAWH2007550, BRAWH2007570, BRAWH2007591, BRAWH2007658, BRAWH2007664, BRAWH2007800, BRAWH2007825, BRAWH2007862,

BRAWH2007890, BRAWH2008128, BRAWH2008219, BRAWH2008255, BRAWH2008292, BRAWH2008297, BRAWH2008366, BRAWH2008380, BRAWH2008577, BRAWH2008790, BRAWH2008903, BRAWH2008993, BRAWH2009112, BRAWH2009227, BRAWH2009261, BRAWH2009263, BRAWH2009298, BRAWH2009393, BRAWH2009485, BRAWH2009490, BRAWH2009558, BRAWH2009590, BRAWH2009626, BRAWH2009695, BRAWH2009907, BRAWH2010000, BRAWH2010069, BRAWH2010084, BRAWH2010136, BRAWH2010318, BRAWH2010364, BRAWH2010536, BRAWH2010552, BRAWH2010584, BRAWH2010618, BRAWH2010619, BRAWH2010754, BRAWH2010980, BRAWH2011066, BRAWH2011079, BRAWH2011155, BRAWH2011166, BRAWH2011204, BRAWH2011286, BRAWH2011294, BRAWH2011400, BRAWH2011762, BRAWH2011795, BRAWH2011796, BRAWH2011812,

BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012538, BRAWH2012540, BRAWH2012619, BRAWH2012698, BRAWH2012749, BRAWH2012827, BRAWH2012866, BRAWH2012963, BRAWH2013047, BRAWH2013748, BRAWH2013866, BRAWH2013914, BRAWH2013941, BRAWH2013955, BRAWH2014234, BRAWH2014379, BRAWH2014414, BRAWH2014473, BRAWH2014511, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016221, BRAWH2016223,

BRAWH2016269, BRAWH2016305, BRAWH2016439, BRAWH2016446, BRAWH2016562, BRAWH2016655, BRAWH2016679, BRAWH2016702, BRAWH2016724, BRAWH2016785, BRAWH2016792, BRAWH2016800, BRAWH2017103, BRAWH2017250, BRAWH2017294, BRAWH2017304, BRAWH2017305, BRAWH2017379, BRAWH2017407, BRAWH2017433, BRAWH2017523, BRAWH2017534, BRAWH2017542, BRAWH2017635, BRAWH2017913, BRAWH2018267, BRAWH2018282, BRAWH2018317, BRAWH2018506, BRAWH2018514, BRAWH2018526, BRAWH2018601, BRAWH2018729, BRAWH2018745, BRAWH2018875, BRAWH2019053, BRAWH2019055, BRAWH2019198, BRAWH3000078, BRAWH3000100, BRAWH3000166, BRAWH3000314, BRAWH3000329, BRAWH3000410, BRAWH3000446, BRAWH3000491, BRAWH3000884, BRAWH3001053, BRAWH3001326, BRAWH3001475,

BRAWH3001636, BRAWH3001638, BRAWH3001712, BRAWH3001760, BRAWH3001783, BRAWH3001833, BRAWH3001879, BRAWH3001891, BRAWH3002433, BRAWH3002467, BRAWH3002513, BRAWH3002574, BRAWH3002600, BRAWH3002819, BRAWH3003019, BRAWH3003136, BRAWH3003244, BRAWH3003343, BRAWH3003522, BRAWH3003598, BRAWH3003801, BRAWH3003975, BRAWH3004222, BRAWH3004335, BRAWH3004453, BRAWH3004666, BRAWH3005037, BRAWH3005047, BRAWH3005132, BRAWH3005292, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007783, BRAWH3008167, BRAWH3008341, BRAWH3008559,

BRAWH3008697, BRAWH3008867, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623, BRAWH3011685, BRAWH3011907, BRAWH3011929, BRAWH3012005, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016271, BRAWH3016715, BRAWH3017259, BRAWH3017260, BRAWH3017477, BRAWH3017537, BRAWH3017980, BRAWH3018369, BRAWH3018548, BRAWH3018969, BRAWH3019026, BRAWH3019529, BRAWH3019594, BRAWH3019629,

BRAWH3019820, BRAWH3020200, BRAWH3020318, BRAWH3020486, BRAWH3020884, BRAWH3020928, BRAWH3021012, BRAWH3021580, BRAWH3021641, BRAWH3021643, BRAWH3021724, BRAWH3022347, BRAWH3022431, BRAWH3022459, BRAWH3022542, BRAWH3022719, BRAWH3022866, BRAWH3022900, BRAWH3023156, BRAWH3023168, BRAWH3023172, BRAWH3023274, BRAWH3023421, BRAWH3024231, BRAWH3024469, BRAWH3024506, BRAWH3024613, BRAWH3026349, BRAWH3026394, BRAWH3026529, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538,

BRAWH3029556, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620, BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035904, BRAWH3035914, BRAWH3036077, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000105, BRCAN1000149, BRCAN1000168, BRCAN2000148, BRCAN2000149, BRCAN2000239, BRCAN2000346,

BRCAN2000364, BRCAN2000383, BRCAN2000418, BRCAN2000523, BRCAN2000541, BRCAN2000563, BRCAN2000577, BRCAN2000620, BRCAN2000639, BRCAN2000763, BRCAN2000832, BRCAN2000923, BRCAN2001223, BRCAN2001866, BRCAN2002416, BRCAN2002473, BRCAN2002562, BRCAN2002662, BRCAN2002826, BRCAN2002854, BRCAN2002856, BRCAN2002892, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119,

BRCAN2007144, BRCAN2007426, BRCAN2007525, BRCAN2007700, BRCAN2008494, BRCAN2008528, BRCAN2008701, BRCAN2008894, BRCAN2009156, BRCAN2009168, BRCAN2009203, BRCAN2010374, BRCAN2010376, BRCAN2010547, BRCAN2010581, BRCAN2010665, BRCAN2011254, BRCAN2011316, BRCAN2011602, BRCAN2011946, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015757, BRCAN2015886, BRCAN2016025, BRCAN2016046, BRCAN2016174, BRCAN2016433, BRCAN2016619, BRCAN2016762, BRCAN2016814,

BRCAN2017442, BRCAN2017905, BRCAN2018240, BRCAN2018269, BRCAN2018490, BRCAN2018566, BRCAN2018667, BRCAN2018748, BRCAN2018935, BRCAN2019002, BRCAN2019387, BRCAN2019773, BRCAN2019907, BRCAN2020234, BRCAN2020331, BRCAN2020467, BRCAN2020710, BRCAN2020880, BRCAN2021028, BRCAN2021325, BRCAN2021452, BRCAN2021669, BRCAN2021718, BRCAN2022191, BRCAN2022472, BRCAN2022474, BRCAN2023347, BRCAN2023724, BRCAN2024451, BRCAN2024563, BRCAN2024572, BRCAN2025009, BRCAN2025093, BRCAN2025349, BRCAN2025368, BRCAN2025712, BRCAN2025982, BRCAN2026117, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028036,

BRCAN2028338, BRCAN2028378, BRCAN2028460, BRCAN2028545, BRCAN2028634, BRCAN2028702, BRCOC1000087, BRCOC2000047, BRCOC2000152, BRCOC2000184, BRCOC2000333, BRCOC2000360, BRCOC2000404, BRCOC2000637, BRCOC2000670, BRCOC2000816, BRCOC2000850, BRCOC2001355, BRCOC2002085, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003513, BRCOC2003740, BRCOC2004036, BRCOC2004175, BRCOC2004354, BRCOC2004455, BRCOC2004950, BRCOC2005542, BRCOC2005903, BRCOC2005951, BRCOC2006189, BRCOC2006243, BRCOC2006270, BRCOC2006639, BRCOC2006942, BRCOC2007034, BRCOC2007593, BRCOC2007733, BRCOC2007769, BRCOC2008064, BRCOC2008225,

BRCOC2008642, BRCOC2008993, BRCOC2009052, BRCOC2009196, BRCOC2009380, BRCOC2009638, BRCOC2009937, BRCOC2010115, BRCOC2010510, BRCOC2010980, BRCOC2010994, BRCOC2011010, BRCOC2011398, BRCOC2011418, BRCOC2011506, BRCOC2011769, BRCOC2011996, BRCOC2012229, BRCOC2012386, BRCOC2012813, BRCOC2013448, BRCOC2013573, BRCOC2013675, BRCOC2013780, BRCOC2014400, BRCOC2014748, BRCOC2014833, BRCOC2015597, BRCOC2015824, BRCOC2015908, BRCOC2015944, BRCOC2016525, BRCOC2016661, BRCOC2017061, BRCOC2017856, BRCOC2018775, BRCOC2018877, BRCOC2019255, BRCOC2019549, BRCOC2019841, BRCOC2019896, BRCOC2019934, BRCOC2020122, BRCOC2020142, BRHIP1000058, BRHIP1000072, BRHIP1000129, BRHIP1000174, BRHIP1000258, BRHIP2000021,

BRHIP2000079, BRHIP2000163, BRHIP2000210, BRHIP2000312, BRHIP2000359, BRHIP2000445, BRHIP2000506, BRHIP2000532, BRHIP2000621, BRHIP2000691, BRHIP2000826, BRHIP2000920, BRHIP2001074, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003242, BRHIP2003272, BRHIP2003748, BRHIP2003786, BRHIP2003828, BRHIP2003917, BRHIP2004312, BRHIP2004355, BRHIP2004814, BRHIP2004883, BRHIP2004902, BRHIP2005010, BRHIP2005236, BRHIP2005354, BRHIP2005600, BRHIP2005719, BRHIP2005724, BRHIP2005752, BRHIP2005932, BRHIP2006617, BRHIP2006782,

BRHIP2006800, BRHIP2006819, BRHIP2006921, BRHIP2007307, BRHIP2007616, BRHIP2007690, BRHIP2007928, BRHIP2007969, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2010217, BRHIP2010309, BRHIP2010444, BRHIP2010487, BRHIP2010610, BRHIP2010723, BRHIP2010744, BRHIP2011080, BRHIP2011120, BRHIP2011491, BRHIP2011576, BRHIP2011616, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447,

BRHIP2013510, BRHIP2013699, BRHIP2013723, BRHIP2013958, BRHIP2013972, BRHIP2014063, BRHIP2014228, BRHIP2014285, BRHIP2014291, BRHIP2014387, BRHIP2014954, BRHIP2015224, BRHIP2015356, BRHIP2015360, BRHIP2016005, BRHIP2016125, BRHIP2016788, BRHIP2016968, BRHIP2016990, BRHIP2017315, BRHIP2017385, BRHIP2017404, BRHIP2017542, BRHIP2017553, BRHIP2017642, BRHIP2017714, BRHIP2017780, BRHIP2018014, BRHIP2018369, BRHIP2018612, BRHIP2018635, BRHIP2018650, BRHIP2018719, BRHIP2018805, BRHIP2018998, BRHIP2019007, BRHIP2019047, BRHIP2019149, BRHIP2019177, BRHIP2019186, BRHIP2019291, BRHIP2019494, BRHIP2019589, BRHIP2019641, BRHIP2019884, BRHIP2019972, BRHIP2020182, BRHIP2020622, BRHIP2020695, BRHIP2020743,

BRHIP2020842, BRHIP2020859, BRHIP2020930, BRHIP2021102, BRHIP2021289, BRHIP2021495, BRHIP2021615, BRHIP2021744, BRHIP2021762, BRHIP2021858, BRHIP2021870, BRHIP2021929, BRHIP2022006, BRHIP2022054, BRHIP2022221, BRHIP2022298, BRHIP2022467, BRHIP2022708, BRHIP2023071, BRHIP2023229, BRHIP2023292, BRHIP2023695, BRHIP2023735, BRHIP2023762, BRHIP2023773, BRHIP2023860, BRHIP2023869, BRHIP2024165, BRHIP2024201, BRHIP2024347, BRHIP2024549, BRHIP2024789, BRHIP2024911, BRHIP2024941, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027054, BRHIP2027563,

BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393, BRHIP2029529, BRHIP2029643, BRHIP2029663, BRHIP3000060, BRHIP3000070, BRHIP3000081, BRHIP3000240, BRHIP3000377, BRHIP3000457, BRHIP3000488, BRHIP3000526, BRHIP3000626, BRHIP3000859, BRHIP3000907, BRHIP3001076, BRHIP3001079, BRHIP3001141, BRHIP3001283, BRHIP3001338, BRHIP3001360, BRHIP3001481, BRHIP3001878, BRHIP3001964, BRHIP3002124, BRHIP3002363, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193,

BRHIP3004215, BRHIP3004416, BRHIP3004710, BRHIP3004725, BRHIP3004774, BRHIP3004786, BRHIP3004863, BRHIP3004968, BRHIP3005037, BRHIP3005137, BRHIP3005142, BRHIP3005231, BRHIP3005307, BRHIP3005574, BRHIP3005768, BRHIP3005801, BRHIP3005944, BRHIP3006279, BRHIP3006390, BRHIP3006449, BRHIP3006683, BRHIP3006717, BRHIP3006786, BRHIP3006950, BRHIP3007172, BRHIP3007183, BRHIP3007195, BRHIP3007239, BRHIP3007291, BRHIP3007409, BRHIP3007424, BRHIP3007483, BRHIP3007586, BRHIP3007609, BRHIP3007640, BRHIP3007960, BRHIP3008082, BRHIP3008183, BRHIP3008314, BRHIP3008320, BRHIP3008344, BRHIP3008565, BRHIP3008598, BRHIP3008606, BRHIP3008691, BRHIP3008714, BRHIP3008997, BRHIP3009099, BRHIP3009140, BRHIP3009181,

BRHIP3009318, BRHIP3009427, BRHIP3009448, BRHIP3009672, BRHIP3009753, BRHIP3010135, BRHIP3010289, BRHIP3010529, BRHIP3011082, BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3013078, BRHIP3013271, BRHIP3013588, BRHIP3013698, BRHIP3013765, BRHIP3013897, BRHIP3014237, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3019643, BRHIP3019824, BRHIP3019880, BRHIP3019956, BRHIP3020155, BRHIP3020182, BRHIP3021019, BRHIP3021499, BRHIP3021534, BRHIP3021778,

BRHIP3021987, BRHIP3022656, BRHIP3023922, BRHIP3024118, BRHIP3024533, BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000175, BRSSN2000293, BRSSN2000312, BRSSN2000518, BRSSN2000561, BRSSN2000566, BRSSN2000715, BRSSN2001213, BRSSN2001275, BRSSN2001342, BRSSN2001426, BRSSN2001503, BRSSN2001758, BRSSN2001810, BRSSN2002857, BRSSN2002859, BRSSN2003086, BRSSN2003093,

BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2006133, BRSSN2006390, BRSSN2006575, BRSSN2006611, BRSSN2006644, BRSSN2007076, BRSSN2007168, BRSSN2007202, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009627, BRSSN2009630, BRSSN2009702, BRSSN2010110, BRSSN2010587, BRSSN2010596, BRSSN2010830, BRSSN2011653, BRSSN2012081, BRSSN2012103, BRSSN2012157, BRSSN2012198, BRSSN2012254, BRSSN2012622, BRSSN2012691, BRSSN2012838, BRSSN2013095, BRSSN2013544, BRSSN2013696, BRSSN2013733,

BRSSN2013753, BRSSN2013874, BRSSN2014112, BRSSN2014294, BRSSN2014424, BRSSN2014513, BRSSN2014556, BRSSN2014573, BRSSN2014636, BRSSN2015199, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017422, BRSSN2017530, BRSSN2017682, BRSSN2017757, BRSSN2018218, BRSSN2018440, BRSSN2018731, BRSSN2018771, BRSSN2018925, BRSTN1000097, BRSTN2000070, BRSTN2000220, BRSTN2000312, BRSTN2000367, BRSTN2000536, BRSTN2000872, BRSTN2001613, BRSTN2001801, BRSTN2002105, BRSTN2002400, BRSTN2002532, BRSTN2002691, BRSTN2003590, BRSTN2003835, BRSTN2004863, BRSTN2004987, BRSTN2005721, BRSTN2006306,

BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012284, BRSTN2012320, BRSTN2012380, BRSTN2013171, BRSTN2013354, BRSTN2014633, BRSTN2014751, BRSTN2014770, BRSTN2015015, BRSTN2016066, BRSTN2016335, BRSTN2016678, BRSTN2016892, BRSTN2016918, BRSTN2017151, BRSTN2017237, BRSTN2017379, BRSTN2017771, BRSTN2017995, BRSTN2018077, BRSTN2018083, BRSTN2018596, BRSTN2018692,

BRSTN2018712, BRSTN2019079, BRSTN2019129, BRTHA1000021, BRTHA1000063, BRTHA2000057, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565, BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004361, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005,

BRTHA2005448, BRTHA2005579, BRTHA2005831, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2008316, BRTHA2008502, BRTHA2008513, BRTHA2008527, BRTHA2008535, BRTHA2008598, BRTHA2008669, BRTHA2008955, BRTHA2009311, BRTHA2009349, BRTHA2009486, BRTHA2009803, BRTHA2009846, BRTHA2009972, BRTHA2010008, BRTHA2010033, BRTHA2010073, BRTHA2010097, BRTHA2010191, BRTHA2010397, BRTHA2010608, BRTHA2010672, BRTHA2010884, BRTHA2010907, BRTHA2011062, BRTHA2011194, BRTHA2011321, BRTHA2011351, BRTHA2011500, BRTHA2011641, BRTHA2012129, BRTHA2012183,

BRTHA2012189, BRTHA2012351, BRTHA2012392, BRTHA2012466, BRTHA2012555, BRTHA2012562, BRTHA2013262, BRTHA2013426, BRTHA2013460, BRTHA2013515, BRTHA2013595, BRTHA2013610, BRTHA2013707, BRTHA2013942, BRTHA2014287, BRTHA2014334, BRTHA2014381, BRTHA2014578, BRTHA2014647, BRTHA2014792, BRTHA2014828, BRTHA2014909, BRTHA2014975, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016765, BRTHA2017178, BRTHA2017353, BRTHA2017364, BRTHA2017972, BRTHA2018011, BRTHA2018129, BRTHA2018165, BRTHA2018304, BRTHA2018344, BRTHA2018420, BRTHA2018443, BRTHA2018558, BRTHA2018591,

BRTHA2018624, BRTHA2018706, BRTHA2019014, BRTHA2019022, BRTHA2019048, BRTHA2019726, BRTHA2019743, BRTHA2020400, BRTHA2020566, BRTHA2020642, BRTHA2020695, BRTHA2020721, BRTHA2020781, BRTHA2020811, BRTHA2020910, BRTHA2021212, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026290, BRTHA2026311, BRTHA2027227, BRTHA2027229, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874,

BRTHA2035448, BRTHA2036055, BRTHA2036295, BRTHA2036660, BRTHA2037247, BRTHA2038279, BRTHA2038345, BRTHA2038353, BRTHA2038396, BRTHA3000252, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002933, BRTHA3002955, BRTHA3003000, BRTHA3003023, BRTHA3003074, BRTHA3003225, BRTHA3003339, BRTHA3003343, BRTHA3003449, BRTHA3003474, BRTHA3003704, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007532, BRTHA3007662, BRTHA3007685,

BRTHA3007769, BRTHA3007922, BRTHA3008143, BRTHA3008293, BRTHA3008310, BRTHA3008386, BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010540, BRTHA3010717, BRTHA3011149, BRTHA3011187, BRTHA3011229, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514,

BRTHA3018617, BRTHA3018623, BRTHA3018656, BRTHA3019048, BRTHA3019105, BRTHA3019183, BRTHA3020000, BRTHA3020369, BRTHA3021569, BRTHA3021708, BRTHA3021786, BRTHA3023403, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026507, BRTHA3026556, BRTHA3026916, BRTHA3027171, BRTHA3027318, BRTHA3027638, BRTHA3027820, BRTHA3027879, BRTHA3027957, BRTHA3028339, BRTHA3028505, BRTHA3028782, CD34C2000051, CD34C2000152, CD34C2000175, CD34C2000443, CD34C3000125, CD34C3000314, CD34C3000424, CD34C3000494, CERVX1000042, CERVX2000812, CERVX2000968, CERVX2002006, CERVX2002013, CERVX2002430, CERVX2002770, CHONS1000058,

CHONS2000172, CHONS2001287, CHONS2001382, CHONS2001797, CHONS2001834, CHONS2002419, COLON1000030, COLON1000084, COLON1000135, COLON2000470, COLON2000557, COLON2000568, COLON2001721, COLON2001829, COLON2001866, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000020, CTONG1000022, CTONG1000040, CTONG1000056, CTONG1000062, CTONG1000086, CTONG1000088, CTONG1000093, CTONG1000097, CTONG1000113, CTONG1000137, CTONG1000165,

CTONG1000180, CTONG1000181, CTONG1000220, CTONG1000277, CTONG1000283, CTONG1000302, CTONG1000341, CTONG1000476, CTONG1000488, CTONG1000508, CTONG1000540, CTONG2000031, CTONG2000034, CTONG2000042, CTONG2000218, CTONG2000279, CTONG2000303, CTONG2000330, CTONG2000395, CTONG2000452, CTONG2000480, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001413, CTONG2001513, CTONG2001524, CTONG2001533, CTONG2001598, CTONG2001681, CTONG2001724, CTONG2001749, CTONG2001800, CTONG2001820, CTONG2001877, CTONG2001911, CTONG2001932, CTONG2001955, CTONG2002066, CTONG2002095, CTONG2002143, CTONG2002417,

CTONG2002418, CTONG2002453, CTONG2002520, CTONG2002558, CTONG2002590, CTONG2002709, CTONG2002766, CTONG2002816, CTONG2002832, CTONG2002841, CTONG2002965, CTONG2002999, CTONG2003028, CTONG2003115, CTONG2003189, CTONG2003245, CTONG2003293, CTONG2003348, CTONG2003350, CTONG2003361, CTONG2003372, CTONG2003375, CTONG2003427, CTONG2003517, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004454, CTONG2004550, CTONG2004669, CTONG2004716, CTONG2004912, CTONG2004941, CTONG2004948, CTONG2005049, CTONG2005110, CTONG2005145, CTONG2005265,

CTONG2005290, CTONG2005374, CTONG2005399, CTONG2005567, CTONG2005585, CTONG2005635, CTONG2005775, CTONG2005795, CTONG2005890, CTONG2005904, CTONG2005913, CTONG2006129, CTONG2006223, CTONG2006273, CTONG2006310, CTONG2006393, CTONG2006412, CTONG2006449, CTONG2006515, CTONG2006524, CTONG2006568, CTONG2006611, CTONG2006620, CTONG2006691, CTONG2006836, CTONG2006942, CTONG2006964, CTONG2007009, CTONG2007091, CTONG2007104, CTONG2007175, CTONG2007259, CTONG2007272, CTONG2007293, CTONG2007297, CTONG2007399, CTONG2007474, CTONG2007613, CTONG2007623, CTONG2007681, CTONG2007779, CTONG2007898, CTONG2007959, CTONG2007970, CTONG2008014, CTONG2008184, CTONG2008262, CTONG2008269, CTONG2008321, CTONG2008343,

CTONG2008402, CTONG2008405, CTONG2008518, CTONG2008521, CTONG2008562, CTONG2008577, CTONG2008595, CTONG2008689, CTONG2008721, CTONG2008773, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423, CTONG2009440, CTONG2009527, CTONG2009529, CTONG2009531, CTONG2009570, CTONG2009572, CTONG2009604, CTONG2009643, CTONG2009675, CTONG2009689, CTONG2009725, CTONG2009766, CTONG2009768, CTONG2009871, CTONG2009887, CTONG2009891, CTONG2009923, CTONG2009955, CTONG2009958, CTONG2010018, CTONG2010024, CTONG2010116, CTONG2010133, CTONG2010148, CTONG2010276, CTONG2010330, CTONG2010508, CTONG2010566,

CTONG2010623, CTONG2010803, CTONG2011188, CTONG2011429, CTONG2011474, CTONG2011676, CTONG2011716, CTONG2011765, CTONG2011779, CTONG2011815, CTONG2011822, CTONG2011920, CTONG2011985, CTONG2012029, CTONG2012077, CTONG2012101, CTONG2012123, CTONG2012158, CTONG2012230, CTONG2012425, CTONG2012447, CTONG2012473, CTONG2012476, CTONG2012533, CTONG2012554, CTONG2012564, CTONG2012724, CTONG2012738, CTONG2012758, CTONG2012843, CTONG2012879, CTONG2012901, CTONG2013128, CTONG2013149, CTONG2013156, CTONG2013178, CTONG2013222, CTONG2013284, CTONG2013339, CTONG2013352, CTONG2013381, CTONG2013393, CTONG2013511, CTONG2013630, CTONG2013803, CTONG2013985, CTONG2013986, CTONG2014032, CTONG2014058, CTONG2014191,

CTONG2014206, CTONG2014234, CTONG2014264, CTONG2014369, CTONG2014630, CTONG2014635, CTONG2014646, CTONG2014705, CTONG2014946, CTONG2014959, CTONG2014966, CTONG2014995, CTONG2015204, CTONG2015316, CTONG2015330, CTONG2015358, CTONG2015434, CTONG2015518, CTONG2015540, CTONG2015596, CTONG2015633, CTONG2015717, CTONG2015804, CTONG2015815, CTONG2015866, CTONG2015914, CTONG2015953, CTONG2016217, CTONG2016355, CTONG2016408, CTONG2016499, CTONG2016559, CTONG2016658, CTONG2016775, CTONG2016797, CTONG2016824, CTONG2016846, CTONG2016904, CTONG2016942, CTONG2016953, CTONG2017021, CTONG2017094, CTONG2017292, CTONG2017409, CTONG2017444, CTONG2017458, CTONG2017604, CTONG2017650, CTONG2017804, CTONG2017814,

CTONG2017910, CTONG2017939, CTONG2017989, CTONG2018062, CTONG2018069, CTONG2018147, CTONG2018217, CTONG2018279, CTONG2018343, CTONG2018379, CTONG2018383, CTONG2018413, CTONG2018483, CTONG2018632, CTONG2018652, CTONG2018653, CTONG2018655, CTONG2018808, CTONG2018900, CTONG2019063, CTONG2019144, CTONG2019232, CTONG2019400, CTONG2019652, CTONG2019833, CTONG2020031, CTONG2020127, CTONG2020171, CTONG2020374, CTONG2020411, CTONG2020445, CTONG2020522, CTONG2020806, CTONG2020831, CTONG2020974, CTONG2021132, CTONG2021168, CTONG2022153, CTONG2022601, CTONG2023021, CTONG2023449, CTONG2023512, CTONG2024206, CTONG2024614, CTONG2024749, CTONG2025496, CTONG2025513, CTONG2025516, CTONG2025610, CTONG2025900,

CTONG2026513, CTONG2026770, CTONG2026920, CTONG2026987, CTONG2027150, CTONG2027263, CTONG2027361, CTONG2027591, CTONG2027736, CTONG2027783, CTONG2027828, CTONG2027959, CTONG2028097, CTONG2028208, CTONG2028486, CTONG2028518, CTONG2028687, CTONG3000686, CTONG3000707, CTONG3000896, CTONG3001420, CTONG3001501, CTONG3001560, CTONG3001605, CTONG3002020, CTONG3002370, CTONG3002412, CTONG3002433, CTONG3002552, CTONG3002588, CTONG3002728, CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003972, CTONG3004317, CTONG3004397, CTONG3004550, CTONG3004607, CTONG3004712, CTONG3004726,

CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009227, CTONG3009239, CTONG3009287, CTONG3009328, CTONG3009385, D3OST1000066, D3OST1000109, D3OST1000238, D3OST1000267, D3OST1000270, D3OST2000184, D3OST2000618, D3OST2000654, D3OST2001328, D3OST2001669, D3OST2002182, D3OST2002223, D3OST2002262, D3OST2002272, D3OST2002417, D3OST2002436, D3OST2002648, D3OST2003024, D3OST2003331,

D3OST2003607, D3OST2003797, D3OST2004637, D3OST3000169, D3OST3000195, D3OST3000388, D3OST3000493, D6OST2000127, D6OST2000169, D6OST2000245, D6OST2000507, D9OST2000031, D9OST2000278, D9OST2000440, D9OST2000869, D9OST2001319, D9OST2001433, D9OST2002397, D9OST2002673, D9OST2003106, D9OST2003108, D9OST2003137, D9OST2003397, D9OST2003580, D9OST2003594, D9OST2003762, D9OST2003989, D9OST2004018, D9OST2004073, D9OST2004417, DFNES1000003, DFNES1000107, DFNES1000185, DFNES2000143, DFNES2000146, DFNES2000153, DFNES2000212, DFNES2000268, DFNES2000292, DFNES2000335, DFNES2000432, DFNES2000457, DFNES2000471, DFNES2000913, DFNES2001091, DFNES2001096, DFNES2001108, DFNES2001177, DFNES2001564, DFNES2001565,

DFNES2001647, DFNES2001800, DFNES2001829, DFNES2002220, DFNES2002509, DFNES2002550, DFNES2002588, DFNES2002817, DFNES2002966, DFNES2003081, DFNES2003192, DFNES2003255, DFNES2004354, DFNES2004383, DFNES2004608, DFNES2004684, DFNES2005266, DFNES2005527, DFNES2005779, DFNES2005875, DFNES2006944, DFNES2007299, DFNES2007641, DFNES2007757, DFNES2008280, DFNES2008881, DFNES2010502, DFNES2011192, DFNES2011239, DFNES2011245, DFNES2011380, ERLTF1000021, ERLTF2000324, ERLTF2000465, FCBBF1000023, FCBBF1000069, FCBBF1000117, FCBBF1000118, FCBBF1000121, FCBBF1000125, FCBBF1000131, FCBBF1000155, FCBBF1000157, FCBBF1000171, FCBBF1000174, FCBBF1000182, FCBBF1000198, FCBBF1000206, FCBBF1000220, FCBBF1000232,

FCBBF1000233, FCBBF1000243, FCBBF1000270, FCBBF1000272, FCBBF1000280, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000403, FCBBF1000437, FCBBF1000449, FCBBF1000476, FCBBF1000506, FCBBF1000546, FCBBF1000550, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000691, FCBBF1000732, FCBBF1000748, FCBBF2000038, FCBBF2000094, FCBBF2000105, FCBBF2000123, FCBBF2000195, FCBBF2000214, FCBBF2000232, FCBBF2000259, FCBBF2000276, FCBBF2000291, FCBBF2000362, FCBBF2000379, FCBBF2000473, FCBBF2000502, FCBBF2000566,

FCBBF2000576, FCBBF2000591, FCBBF2000677, FCBBF2000678, FCBBF2000685, FCBBF2000733, FCBBF2000808, FCBBF2000815, FCBBF2000825, FCBBF2000940, FCBBF2000951, FCBBF2001001, FCBBF2001105, FCBBF2001116, FCBBF2001188, FCBBF2001211, FCBBF2001238, FCBBF2001243, FCBBF2001299, FCBBF2001427, FCBBF2001480, FCBBF2001529, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002370, FCBBF2002541, FCBBF2002626, FCBBF2002897, FCBBF2002898, FCBBF2002904, FCBBF2002953, FCBBF2003083, FCBBF2003102, FCBBF2003269, FCBBF2003293, FCBBF2003297, FCBBF2003395, FCBBF2003528, FCBBF2003543, FCBBF2003549, FCBBF2003567, FCBBF2003636,

FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005966, FCBBF2006131, FCBBF2006380, FCBBF2006418, FCBBF2006452, FCBBF2006634, FCBBF2006781, FCBBF2007012, FCBBF2007186, FCBBF2007265, FCBBF2007302, FCBBF2007610, FCBBF2007633, FCBBF2007840, FCBBF3000102, FCBBF3000120, FCBBF3000168, FCBBF3000175, FCBBF3000227, FCBBF3000229, FCBBF3000233, FCBBF3000269, FCBBF3000361, FCBBF3000367, FCBBF3000388, FCBBF3000462, FCBBF3000473, FCBBF3000501, FCBBF3000518,

FCBBF3000550, FCBBF3000847, FCBBF3001018, FCBBF3001020, FCBBF3001235, FCBBF3001281, FCBBF3001377, FCBBF3001594, FCBBF3001632, FCBBF3001657, FCBBF3001855, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557, FCBBF3003787, FCBBF3003902, FCBBF3004021, FCBBF3004041, FCBBF3004261, FCBBF3004323, FCBBF3004390, FCBBF3004409, FCBBF3004502, FCBBF3004842, FCBBF3004847, FCBBF3004955, FCBBF3005160, FCBBF3005218, FCBBF3005444, FCBBF3005698,

FCBBF3005729, FCBBF3006171, FCBBF3006249, FCBBF3006288, FCBBF3006399, FCBBF3006628, FCBBF3007077, FCBBF3007150, FCBBF3007152, FCBBF3007242, FCBBF3007248, FCBBF3007453, FCBBF3007462, FCBBF3007540, FCBBF3007597, FCBBF3007604, FCBBF3007631, FCBBF3007829, FCBBF3007855, FCBBF3007859, FCBBF3007860, FCBBF3008073, FCBBF3008100, FCBBF3008159, FCBBF3008251, FCBBF3008311, FCBBF3008362, FCBBF3008475, FCBBF3008556, FCBBF3008644, FCBBF3008689, FCBBF3008748, FCBBF3008870, FCBBF3008938, FCBBF3008944, FCBBF3009069, FCBBF3009101, FCBBF3009152, FCBBF3009317, FCBBF3009428, FCBBF3009464, FCBBF3009479, FCBBF3009526, FCBBF3009703, FCBBF3009717, FCBBF3009888, FCBBF3009949, FCBBF3009978, FCBBF3010008, FCBBF3010012,

FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010729, FCBBF3010733, FCBBF3011003, FCBBF3011418, FCBBF3011485, FCBBF3011570, FCBBF3011592, FCBBF3011867, FCBBF3011889, FCBBF3012170, FCBBF3012182, FCBBF3012288, FCBBF3012332, FCBBF3012443, FCBBF3012588, FCBBF3012667, FCBBF3012842, FCBBF3012901, FCBBF3013041, FCBBF3013058, FCBBF3013205, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809,

FCBBF3015870, FCBBF3016018, FCBBF3016134, FCBBF3016178, FCBBF3016451, FCBBF3016618, FCBBF3016622, FCBBF3016644, FCBBF3016987, FCBBF3017059, FCBBF3017071, FCBBF3017123, FCBBF3017233, FCBBF3017255, FCBBF3017288, FCBBF3017396, FCBBF3017535, FCBBF3017681, FCBBF3017729, FCBBF3017873, FCBBF3017974, FCBBF3018067, FCBBF3018173, FCBBF3018453, FCBBF3018591, FCBBF3018753, FCBBF3018826, FCBBF3018949, FCBBF3019085, FCBBF3019437, FCBBF3019455, FCBBF3019564, FCBBF3019569, FCBBF3019570, FCBBF3019663, FCBBF3019714, FCBBF3019716, FCBBF3019784, FCBBF3019867, FCBBF3019961, FCBBF3020030, FCBBF3020138, FCBBF3020163, FCBBF3020232, FCBBF3020599, FCBBF3021026, FCBBF3021191, FCBBF3021221, FCBBF3021501, FCBBF3021506,

FCBBF3021524, FCBBF3021807, FCBBF3021940, FCBBF3021985, FCBBF3022005, FCBBF3022291, FCBBF3022311, FCBBF3022566, FCBBF3022765, FCBBF3022894, FCBBF3023061, FCBBF3023078, FCBBF3023372, FCBBF3023443, FCBBF3023599, FCBBF3023704, FCBBF3023887, FCBBF3023895, FCBBF3024096, FCBBF3024225, FCBBF3024266, FCBBF3024364, FCBBF3024623, FCBBF3024911, FCBBF3025098, FCBBF3025252, FCBBF3025280, FCBBF3025285, FCBBF3025417, FCBBF3025528, FCBBF3025568, FCBBF3025650, FCBBF3025674, FCBBF3025730, FCBBF3026021, FCBBF3026048, FCBBF3026251, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102,

FCBBF3028143, FCBBF3028188, FCBBF3028472, FCBBF3028528, FCBBF3028582, FCBBF3028671, FCBBF3028794, FCBBF4000061, FCBBF4000076, FCBBF4000142, FCBBF4000173, FCBBF4000192, FCBBF4000193, FCBBF4000268, FCBBF4000282, FCBBF4000399, FCBBF4000415, FCBBF4000500, FCBBF4000529, FCBBF5000041, FCBBF5000061, FCBBF5000165, FCBBF5000325, FCBBF5000353, FCBBF5000384, FCBBF5000483, FCBBF5000495, FEBRA1000005, FEBRA1000008, FEBRA1000022, FEBRA1000030, FEBRA1000088, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000220, FEBRA2000282, FEBRA2000286, FEBRA2000297, FEBRA2000311, FEBRA2000321,

FEBRA2000333, FEBRA2000378, FEBRA2000391, FEBRA2000394, FEBRA2000397, FEBRA2000399, FEBRA2000404, FEBRA2000409, FEBRA2000415, FEBRA2000436, FEBRA2000462, FEBRA2000468, FEBRA2000491, FEBRA2000504, FEBRA2000538, FEBRA2000575, FEBRA2000581, FEBRA2000656, FEBRA2000659, FEBRA2000680, FEBRA2000690, FEBRA2000727, FEBRA2000734, FEBRA2000762, FEBRA2000771, FEBRA2000772, FEBRA2000782, FEBRA2000790, FEBRA2000793, FEBRA2000803, FEBRA2000805, FEBRA2000809, FEBRA2000856, FEBRA2000862, FEBRA2000877, FEBRA2000880, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133,

FEBRA2001161, FEBRA2001175, FEBRA2001197, FEBRA2001217, FEBRA2001245, FEBRA2001267, FEBRA2001334, FEBRA2001351, FEBRA2001383, FEBRA2001438, FEBRA2001440, FEBRA2001487, FEBRA2001561, FEBRA2001584, FEBRA2001590, FEBRA2001591, FEBRA2001610, FEBRA2001620, FEBRA2001659, FEBRA2001705, FEBRA2001706, FEBRA2001752, FEBRA2001772, FEBRA2001790, FEBRA2001914, FEBRA2002009, FEBRA2002027, FEBRA2002086, FEBRA2002109, FEBRA2002191, FEBRA2002260, FEBRA2002399, FEBRA2002410, FEBRA2002442, FEBRA2002508, FEBRA2002525, FEBRA2002527, FEBRA2002552, FEBRA2002611, FEBRA2002628, FEBRA2002662, FEBRA2002682, FEBRA2002727, FEBRA2002781, FEBRA2002783, FEBRA2002818, FEBRA2002858, FEBRA2002882, FEBRA2002897, FEBRA2002908,

FEBRA2002931, FEBRA2002962, FEBRA2002986, FEBRA2003054, FEBRA2003100, FEBRA2003128, FEBRA2003155, FEBRA2003163, FEBRA2003198, FEBRA2003253, FEBRA2003275, FEBRA2003308, FEBRA2003327, FEBRA2003429, FEBRA2003454, FEBRA2003468, FEBRA2003470, FEBRA2003520, FEBRA2003524, FEBRA2003541, FEBRA2003675, FEBRA2003707, FEBRA2003711, FEBRA2003822, FEBRA2003897, FEBRA2003907, FEBRA2004023, FEBRA2004026, FEBRA2004029, FEBRA2004053, FEBRA2004056, FEBRA2004091, FEBRA2004110, FEBRA2004191, FEBRA2004202, FEBRA2004219, FEBRA2004224, FEBRA2004237, FEBRA2004268, FEBRA2004325, FEBRA2004329, FEBRA2004412, FEBRA2004443, FEBRA2004490, FEBRA2004538, FEBRA2004592, FEBRA2004620, FEBRA2004651, FEBRA2004767, FEBRA2004813,

FEBRA2004840, FEBRA2004852, FEBRA2004867, FEBRA2004914, FEBRA2004933, FEBRA2004987, FEBRA2005014, FEBRA2005036, FEBRA2005079, FEBRA2005216, FEBRA2005377, FEBRA2005380, FEBRA2005427, FEBRA2005458, FEBRA2005701, FEBRA2005726, FEBRA2005752, FEBRA2005788, FEBRA2005998, FEBRA2006055, FEBRA2006061, FEBRA2006112, FEBRA2006150, FEBRA2006165, FEBRA2006315, FEBRA2006346, FEBRA2006354, FEBRA2006370, FEBRA2006379, FEBRA2006396, FEBRA2006409, FEBRA2006418, FEBRA2006427, FEBRA2006476, FEBRA2006519, FEBRA2006521, FEBRA2006627, FEBRA2006664, FEBRA2006667, FEBRA2006726, FEBRA2006736, FEBRA2006793, FEBRA2006817, FEBRA2006890, FEBRA2006942, FEBRA2006977, FEBRA2007017, FEBRA2007176, FEBRA2007200, FEBRA2007247,

FEBRA2007280, FEBRA2007414, FEBRA2007436, FEBRA2007449, FEBRA2007458, FEBRA2007534, FEBRA2007551, FEBRA2007566, FEBRA2007589, FEBRA2007620, FEBRA2007708, FEBRA2007714, FEBRA2007793, FEBRA2007818, FEBRA2007823, FEBRA2007931, FEBRA2007957, FEBRA2008009, FEBRA2008086, FEBRA2008134, FEBRA2008135, FEBRA2008159, FEBRA2008165, FEBRA2008172, FEBRA2008210, FEBRA2008255, FEBRA2008265, FEBRA2008282, FEBRA2008311, FEBRA2008360, FEBRA2008459, FEBRA2008468, FEBRA2008475, FEBRA2008481, FEBRA2008542, FEBRA2008660, FEBRA2008681, FEBRA2008741, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029, FEBRA2009074, FEBRA2009117,

FEBRA2009120, FEBRA2009162, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009437, FEBRA2009478, FEBRA2009507, FEBRA2009541, FEBRA2009567, FEBRA2009588, FEBRA2009731, FEBRA2009754, FEBRA2009846, FEBRA2010141, FEBRA2010299, FEBRA2010484, FEBRA2010719, FEBRA2010746, FEBRA2010768, FEBRA2010802, FEBRA2011063, FEBRA2011356, FEBRA2011392, FEBRA2011414, FEBRA2011593, FEBRA2011665, FEBRA2012090, FEBRA2012195, FEBRA2012397, FEBRA2012418, FEBRA2012507, FEBRA2012625, FEBRA2013019, FEBRA2013069, FEBRA2013274, FEBRA2013465, FEBRA2013570, FEBRA2013905, FEBRA2013951, FEBRA2014122, FEBRA2014198, FEBRA2014443,

FEBRA2014939, FEBRA2015076, FEBRA2015175, FEBRA2015292, FEBRA2015588, FEBRA2016112, FEBRA2016398, FEBRA2016583, FEBRA2016621, FEBRA2016654, FEBRA2016739, FEBRA2017098, FEBRA2017138, FEBRA2017154, FEBRA2017200, FEBRA2017223, FEBRA2017333, FEBRA2017502, FEBRA2017533, FEBRA2017736, FEBRA2017780, FEBRA2017811, FEBRA2017850, FEBRA2018051, FEBRA2018203, FEBRA2018407, FEBRA2018433, FEBRA2019172, FEBRA2019298, FEBRA2019389, FEBRA2019457, FEBRA2019519, FEBRA2019582, FEBRA2019637, FEBRA2019711, FEBRA2019867, FEBRA2020406, FEBRA2020566, FEBRA2020582, FEBRA2020668, FEBRA2020801, FEBRA2020845, FEBRA2020886, FEBRA2020977, FEBRA2021134, FEBRA2021171, FEBRA2021339, FEBRA2021431, FEBRA2021497, FEBRA2021550,

FEBRA2021571, FEBRA2021636, FEBRA2021864, FEBRA2021892, FEBRA2021908, FEBRA2021966, FEBRA2022013, FEBRA2022055, FEBRA2022148, FEBRA2022204, FEBRA2022255, FEBRA2022322, FEBRA2022601, FEBRA2022696, FEBRA2022729, FEBRA2022787, FEBRA2022911, FEBRA2022956, FEBRA2022963, FEBRA2022972, FEBRA2023285, FEBRA2023351, FEBRA2023377, FEBRA2023399, FEBRA2023498, FEBRA2023550, FEBRA2023721, FEBRA2023989, FEBRA2023990, FEBRA2024150, FEBRA2024343, FEBRA2024570, FEBRA2024693, FEBRA2024727, FEBRA2024744, FEBRA2024797, FEBRA2024987, FEBRA2025249, FEBRA2025463, FEBRA2025477, FEBRA2026474, FEBRA2026582, FEBRA2026629, FEBRA2026769, FEBRA2026879, FEBRA2026890, FEBRA2026977, FEBRA2026984, FEBRA2027082, FEBRA2027297,

FEBRA2027352, FEBRA2027364, FEBRA2027609, FEBRA2028222, FEBRA2028366, FEBRA2028457, FEBRA2028477, FEBRA2028503, FEHRT2000325, FEHRT2000364, FELNG2000241, HCASM1000021, HCASM1000050, HCASM1000061, HCASM1000130, HCASM2000156, HCASM2000202, HCASM2000214, HCASM2000266, HCASM2000307, HCASM2000534, HCASM2000536, HCASM2000560, HCASM2000871, HCASM2001285, HCASM2001810, HCASM2001866, HCASM2001890, HCASM2002148, HCASM2002502, HCASM2002754, HCASM2002918, HCASM2003076, HCASM2003099, HCASM2003212, HCASM2003237, HCASM2003357, HCASM2003415, HCASM2005484, HCASM2006131, HCASM2006338, HCASM2006359, HCASM2006632, HCASM2007047, HCASM2007317, HCASM2007737, HCASM2007773, HCASM2008154, HCASM2008536, HCASM2009405,

HCASM2009424, HCASM2009463, HCASM2009588, HCHON1000015, HCHON1000131, HCHON1000142, HCHON1000176, HCHON1000191, HCHON2000038, HCHON2000100, HCHON2000156, HCHON2000199, HCHON2000212, HCHON2000244, HCHON2000271, HCHON2000315, HCHON2000322, HCHON2000344, HCHON2000373, HCHON2000395, HCHON2000417, HCHON2000418, HCHON2000475, HCHON2000503, HCHON2000660, HCHON2000663, HCHON2000673, HCHON2000676, HCHON2000705, HCHON2000815, HCHON2000817, HCHON2000818, HCHON2000819, HCHON2000851, HCHON2000898, HCHON2000935, HCHON2000942, HCHON2000956, HCHON2000994, HCHON2001050, HCHON2001099, HCHON2001108, HCHON2001110, HCHON2001116, HCHON2001161, HCHON2001214, HCHON2001233, HCHON2001269, HCHON2001407, HCHON2001450,

HCHON2001453, HCHON2001497, HCHON2001505, HCHON2001519, HCHON2001523, HCHON2001535, HCHON2001546, HCHON2001548, HCHON2001577, HCHON2001598, HCHON2001604, HCHON2001607, HCHON2001646, HCHON2001768, HCHON2001801, HCHON2002123, HCHON2002194, HCHON2002241, HCHON2002496, HCHON2002676, HCHON2002770, HCHON2002964, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005921, HCHON2006714, HCHON2006722, HCHON2006770, HCHON2006786, HCHON2006841, HCHON2006871, HCHON2007046, HCHON2007482, HCHON2007674,

HCHON2007774, HCHON2007881, HCHON2008112, HCHON2008239, HCHON2008444, HCHON2008582, HCHON2008672, HCHON2008871, HCHON2008989, HCHON2009191, HCHON2009384, HCHON2009749, HCHON2009766, HCHON2009795, HCHON2010074, HCHON2010543, HEART1000010, HEART1000088, HEART1000099, HEART1000102, HEART1000118, HEART1000132, HEART1000139, HEART1000142, HEART1000151, HEART1000173, HEART1000185, HEART2000024, HEART2000035, HEART2000099, HEART2000298, HEART2000306, HEART2000309, HEART2000411, HEART2000418, HEART2000444, HEART2000448, HEART2000492, HEART2000520, HEART2000541, HEART2000568, HEART2000611, HEART2000959, HEART2001680, HEART2001756, HEART2002129, HEART2002184, HEART2002220, HEART2002432, HEART2002531,

HEART2002598, HEART2002717, HEART2003027, HEART2003168, HEART2003432, HEART2003440, HEART2003781, HEART2003836, HEART2003852, HEART2004570, HEART2004764, HEART2004931, HEART2004940, HEART2004941, HEART2004980, HEART2005244, HEART2005310, HEART2005581, HEART2005610, HEART2006131, HEART2006310, HEART2006334, HEART2006487, HEART2006557, HEART2006787, HEART2006789, HEART2006909, HEART2007031, HEART2007443, HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000114, HHDPC1000118, HHDPC2000102, HHDPC2000115, HHDPC2000258,

HHDPC2000315, HHDPC2000462, HHDPC2000656, HHDPC2000692, HHDPC2001337, HHDPC2001432, HHDPC2001896, HHDPC2002963, HHDPC2003049, HHDPC2003113, HHDPC2003381, HHDPC2003472, HHDPC2004757, HHDPC2005185, HHDPC2005513, HHDPC2005794, HHDPC2006460, HHDPC2006862, HHDPC2006927, HHDPC2007302, HHDPC2007775, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009528, HLUNG1000017, HLUNG1000024, HLUNG1000030, HLUNG1000034, HLUNG1000053, HLUNG1000064, HLUNG1000084, HLUNG1000091, HLUNG1000104, HLUNG1000105, HLUNG1000110, HLUNG1000151, HLUNG1000169, HLUNG2000014, HLUNG2000027, HLUNG2000068, HLUNG2000116, HLUNG2000130,

HLUNG2000142, HLUNG2000174, HLUNG2000217, HLUNG2000232, HLUNG2000255, HLUNG2000281, HLUNG2000314, HLUNG2000315, HLUNG2000340, HLUNG2000362, HLUNG2000363, HLUNG2000431, HLUNG2000480, HLUNG2000489, HLUNG2000501, HLUNG2000549, HLUNG2000557, HLUNG2000570, HLUNG2000761, HLUNG2000777, HLUNG2000870, HLUNG2000926, HLUNG2000955, HLUNG2000970, HLUNG2001013, HLUNG2001126, HLUNG2001144, HLUNG2001214, HLUNG2001459, HLUNG2001507, HLUNG2001814, HLUNG2001996, HLUNG2002006, HLUNG2002050, HLUNG2002085, HLUNG2002145, HLUNG2002295, HLUNG2002303, HLUNG2002334, HLUNG2002405, HLUNG2002429, HLUNG2002562, HLUNG2002659, HLUNG2002707, HLUNG2002811, HLUNG2002916, HLUNG2002942, HLUNG2002949, HLUNG2002958, HLUNG2002982,

HLUNG2003049, HLUNG2003061, HLUNG2003162, HLUNG2003246, HLUNG2003331, HLUNG2003335, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004037, HLUNG2004067, HLUNG2004170, HLUNG2004217, HLUNG2004273, HLUNG2004388, HLUNG2004416, HLUNG2004521, HLUNG2004684, HLUNG2004707, HLUNG2004774, HLUNG2004775, HLUNG2005012, HLUNG2005030, HLUNG2005045, HLUNG2005076, HLUNG2005133, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094, HLUNG2006397, HLUNG2006599, HLUNG2006614, HLUNG2006671, HLUNG2006781, HLUNG2006843,

HLUNG2006852, HLUNG2006935, HLUNG2007041, HLUNG2007163, HLUNG2007210, HLUNG2007245, HLUNG2007433, HLUNG2007931, HLUNG2008066, HLUNG2008139, HLUNG2008225, HLUNG2008235, HLUNG2008247, HLUNG2008333, HLUNG2008348, HLUNG2008384, HLUNG2008396, HLUNG2008439, HLUNG2008479, HLUNG2008480, HLUNG2008521, HLUNG2008833, HLUNG2008875, HLUNG2008885, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2012049, HLUNG2012287, HLUNG2012315, HLUNG2012546, HLUNG2012600, HLUNG2012727, HLUNG2013097, HLUNG2013204, HLUNG2013304, HLUNG2013350, HLUNG2013622,

HLUNG2013643, HLUNG2013784, HLUNG2013851, HLUNG2014262, HLUNG2014288, HLUNG2014449, HLUNG2015548, HLUNG2015578, HLUNG2015617, HLUNG2016022, HLUNG2016862, HLUNG2017286, HLUNG2017307, HLUNG2017350, HLUNG2017806, HLUNG2018029, HLUNG2018282, HLUNG2018626, HLUNG2019058, HSYRA1000131, HSYRA1000137, HSYRA1000148, HSYRA2000135, HSYRA2000137, HSYRA2000190, HSYRA2000221, HSYRA2000237, HSYRA2000332, HSYRA2000371, HSYRA2000507, HSYRA2000510, HSYRA2000605, HSYRA2000706, HSYRA2000760, HSYRA2000792, HSYRA2000832, HSYRA2000927, HSYRA2001003, HSYRA2001142, HSYRA2001153, HSYRA2001232, HSYRA2001255, HSYRA2001332, HSYRA2001484, HSYRA2001552, HSYRA2001574, HSYRA2001580, HSYRA2001615, HSYRA2001631, HSYRA2002103,

HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007650, HSYRA2007667, HSYRA2008154, HSYRA2008714, HSYRA2009102, IMR321000155, IMR321000158, IMR321000165, IMR321000193, IMR321000207, IMR321000230, IMR321000242, IMR322000010, IMR322000072, IMR322000107, IMR322000144, IMR322000152, IMR322000164, IMR322000302, IMR322000379, IMR322000383, IMR322000418, IMR322000518, IMR322000555, IMR322000604, IMR322000609, IMR322000656, IMR322000672, IMR322000742, IMR322000775, IMR322000791, IMR322000811, IMR322000819, IMR322000838, IMR322000859, IMR322000863, IMR322000917,

IMR322000919, IMR322000953, IMR322000984, IMR322001018, IMR322001049, IMR322001167, IMR322001185, IMR322001218, IMR322001245, IMR322001317, IMR322001318, IMR322001322, IMR322001332, IMR322001380, IMR322001438, IMR322001452, IMR322001491, IMR322001534, IMR322001665, IMR322001670, IMR322001724, IMR322001879, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731,

IMR322015523, IMR322016146, IMR322017049, IMR322018117, IMR322018192, KIDNE1000008, KIDNE1000012, KIDNE1000015, KIDNE1000028, KIDNE1000036, KIDNE1000064, KIDNE1000121, KIDNE1000143, KIDNE1000152, KIDNE2000026, KIDNE2000041, KIDNE2000046, KIDNE2000051, KIDNE2000070, KIDNE2000167, KIDNE2000174, KIDNE2000192, KIDNE2000227, KIDNE2000244, KIDNE2000245, KIDNE2000287, KIDNE2000315, KIDNE2000317, KIDNE2000328, KIDNE2000330, KIDNE2000335, KIDNE2000375, KIDNE2000394, KIDNE2000422, KIDNE2000493, KIDNE2000497, KIDNE2000510, KIDNE2000513, KIDNE2000519, KIDNE2000555, KIDNE2000624, KIDNE2000665, KIDNE2000678, KIDNE2000717, KIDNE2000721, KIDNE2000722, KIDNE2000777, KIDNE2000801, KIDNE2000832, KIDNE2000833,

KIDNE2000846, KIDNE2000867, KIDNE2000909, KIDNE2001117, KIDNE2001140, KIDNE2001269, KIDNE2001373, KIDNE2001802, KIDNE2001840, KIDNE2001857, KIDNE2001873, KIDNE2001897, KIDNE2001931, KIDNE2001979, KIDNE2002015, KIDNE2002162, KIDNE2002168, KIDNE2002191, KIDNE2002198, KIDNE2002262, KIDNE2002265, KIDNE2002438, KIDNE2002483, KIDNE2002768, KIDNE2002795, KIDNE2002798, KIDNE2002839, KIDNE2002845, KIDNE2002872, KIDNE2002882, KIDNE2002980, KIDNE2002991, KIDNE2003185, KIDNE2003188, KIDNE2003233, KIDNE2003335, KIDNE2003377, KIDNE2003507, KIDNE2003752, KIDNE2003764, KIDNE2003837, KIDNE2004034, KIDNE2004054, KIDNE2004084, KIDNE2004095, KIDNE2004115, KIDNE2004233, KIDNE2004262, KIDNE2004279, KIDNE2004294,

KIDNE2004305, KIDNE2004394, KIDNE2004519, KIDNE2004520, KIDNE2004562, KIDNE2004579, KIDNE2004626, KIDNE2004681, KIDNE2004828, KIDNE2004846, KIDNE2004879, KIDNE2004981, KIDNE2004985, KIDNE2004999, KIDNE2005038, KIDNE2005042, KIDNE2005062, KIDNE2005266, KIDNE2005296, KIDNE2005321, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006030, KIDNE2006039, KIDNE2006053, KIDNE2006062, KIDNE2006092, KIDNE2006145, KIDNE2006149, KIDNE2006210, KIDNE2006299, KIDNE2006334, KIDNE2006376, KIDNE2006378, KIDNE2006580, KIDNE2006687, KIDNE2006733, KIDNE2006760,

KIDNE2006820, KIDNE2007005, KIDNE2007040, KIDNE2007077, KIDNE2007186, KIDNE2007222, KIDNE2007356, KIDNE2007422, KIDNE2007569, KIDNE2007810, KIDNE2007811, KIDNE2007910, KIDNE2007944, KIDNE2008022, KIDNE2008069, KIDNE2008072, KIDNE2008116, KIDNE2008117, KIDNE2008218, KIDNE2008315, KIDNE2008362, KIDNE2008378, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2009019, KIDNE2009109, KIDNE2009180, KIDNE2009191, KIDNE2009332, KIDNE2009426, KIDNE2009467, KIDNE2009490, KIDNE2009553, KIDNE2009628, KIDNE2009647, KIDNE2009676, KIDNE2009723,

KIDNE2010007, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011752, KIDNE2011782, KIDNE2011858, KIDNE2012009, KIDNE2012025, KIDNE2012188, KIDNE2012291, KIDNE2012316, KIDNE2012361, KIDNE2012453, KIDNE2012563, KIDNE2012601, KIDNE2012613, KIDNE2012745, KIDNE2012775, KIDNE2012784, KIDNE2012945, KIDNE2013045, KIDNE2013095, KIDNE2013218, KIDNE2013263, KIDNE2013346, KIDNE2013388, KIDNE2013413, KIDNE2013489, KIDNE2013639, KIDNE2013731, KIDNE2013734,

KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014415, KIDNE2014489, KIDNE2014496, KIDNE2014661, KIDNE2014717, KIDNE2014789, KIDNE2014890, KIDNE2014978, KIDNE2015073, KIDNE2015221, KIDNE2015244, KIDNE2015313, KIDNE2015433, KIDNE2015483, KIDNE2015515, KIDNE2015556, KIDNE2015660, KIDNE2015710, KIDNE2015987, KIDNE2016000, KIDNE2016036, KIDNE2016096, KIDNE2016101, KIDNE2016142, KIDNE2016188, KIDNE2016388, KIDNE2016464, KIDNE2016539, KIDNE2016570, KIDNE2016918, KIDNE2017007, KIDNE2017151, KIDNE2017153, KIDNE2017315, KIDNE2017332, KIDNE2017343, KIDNE2017454, KIDNE2017603, KIDNE2017956,

KIDNE2018071, KIDNE2018166, KIDNE2018167, KIDNE2018217, KIDNE2018254, KIDNE2018268, KIDNE2018269, KIDNE2018287, KIDNE2018294, KIDNE2018462, KIDNE2018617, KIDNE2018678, KIDNE2018727, KIDNE2018863, KIDNE2018989, KIDNE2018996, KIDNE2019074, KIDNE2019187, LIVER1000050, LIVER1000055, LIVER1000058, LIVER1000063, LIVER1000073, LIVER1000082, LIVER1000099, LIVER1000126, LIVER1000139, LIVER1000155, LIVER1000165, LIVER1000175, LIVER1000224, LIVER1000278, LIVER1000397, LIVER1000421, LIVER1000433, LIVER1000479, LIVER1000482, LIVER1000542, LIVER2000003, LIVER2000033, LIVER2000158, LIVER2000216, LIVER2000237, LIVER2000247, LIVER2000360, LIVER2000416, LIVER2000446, LIVER2000515, LIVER2000592, LIVER2000626,

LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001835, LIVER2002638, LIVER2002644, LIVER2002660, LIVER2002702, LIVER2003008, LIVER2003234, LIVER2003511, LIVER2003524, LIVER2003568, LIVER2003581, LIVER2003800, LIVER2003854, LIVER2004074, LIVER2004392, LIVER2004565, LIVER2005028, LIVER2005198, LIVER2005218, LIVER2005376, LIVER2005385, LIVER2005512, LIVER2005520, LIVER2005527, LIVER2005544, LIVER2005625, LIVER2005728, LIVER2005973, LIVER2005981, LIVER2006251, LIVER2006410, LIVER2006469, LIVER2006644, LIVER2006764, LIVER2007415, LIVER2007548, LIVER2007568,

LIVER2007721, LIVER2008053, LIVER2008465, LIVER2008473, LIVER2008580, LIVER2008751, LIVER2008945, LIVER2008998, LIVER2009110, LIVER2009118, LIVER2009554, LYMPB1000141, LYMPB1000158, LYMPB2000083, MAMGL1000096, MAMGL1000100, MAMGL1000132, MAMGL1000151, MAMGL1000182, MAMGL1000184, MESAN1000032, MESAN1000035, MESAN1000080, MESAN1000121, MESAN1000147, MESAN1000180, MESAN2000023, MESAN2000026, MESAN2000067, MESAN2000167, MESAN2000264, MESAN2000291, MESAN2000337, MESAN2000434, MESAN2000457, MESAN2000462, MESAN2000501, MESAN2000620, MESAN2000711, MESAN2000894, MESAN2000909, MESAN2001154, MESAN2001770, MESAN2001979, MESAN2001980, MESAN2002113, MESAN2002122, MESAN2002147, MESAN2002186, MESAN2002424,

MESAN2002519, MESAN2002687, MESAN2002709, MESAN2002844, MESAN2002978, MESAN2003037, MESAN2003039, MESAN2003101, MESAN2003322, MESAN2003490, MESAN2003529, MESAN2003646, MESAN2003662, MESAN2003709, MESAN2003851, MESAN2004138, MESAN2004288, MESAN2004575, MESAN2005371, MESAN2005724, MESAN2005808, MESAN2005811, MESAN2006022, MESAN2006043, MESAN2006328, MESAN2006580, MESAN2006599, MESAN2006743, MESAN2006953, MESAN2006971, MESAN2007032, MESAN2007440, MESAN2007597, MESAN2008222, MESAN2008334, MESAN2008415, MESAN2008460, MESAN2008536, MESAN2008679, MESAN2008729, MESAN2008821, MESAN2009019, MESAN2009081, MESAN2009105, MESAN2009311, MESAN2009418, MESAN2009503, MESAN2009522, MESAN2010031, MESAN2010114,

MESAN2010312, MESAN2010321, MESAN2010681, MESAN2011545, MESAN2011597, MESAN2011627, MESAN2011632, MESAN2011977, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013936, MESAN2014295, MESAN2014298, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016965, MESAN2017152, MESAN2017373, MESAN2017417, MESAN2018209, MESAN2018576, MESAN2018670, MESAN2018699, MESTC1000042, MESTC1000147, MESTC2000150, MESTC2000153, MESTC2000170, NB9N41000011, NB9N41000047, NB9N41000135,

NB9N41000142, NB9N41000146, NB9N42000122, NB9N42000196, NB9N42000281, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000744, NESOP2001144, NESOP2001433, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000187, NHNPC2000212, NHNPC2000606, NHNPC2000877, NHNPC2001108, NHNPC2001223, NHNPC2001312, NHNPC2001816, NHNPC2001931, NHNPC2002565, NHNPC2002749, NOVAR1000102, NOVAR2000136,

NOVAR2000352, NOVAR2000412, NOVAR2000783, NOVAR2000962, NOVAR2001108, NOVAR2001620, NOVAR2001783, NOVAR2001876, NT2NE1000004, NT2NE1000018, NT2NE1000023, NT2NE1000024, NT2NE1000059, NT2NE1000063, NT2NE1000073, NT2NE1000083, NT2NE1000120, NT2NE1000175, NT2NE1000185, NT2NE2000038, NT2NE2000054, NT2NE2000057, NT2NE2000064, NT2NE2000109, NT2NE2000137, NT2NE2000156, NT2NE2000222, NT2NE2000259, NT2NE2000284, NT2NE2000299, NT2NE2000327, NT2NE2000353, NT2NE2000369, NT2NE2000374, NT2NE2000386, NT2NE2000390, NT2NE2000455, NT2NE2000488, NT2NE2000517, NT2NE2000546, NT2NE2000548, NT2NE2000550, NT2NE2000586, NT2NE2000612, NT2NE2000636, NT2NE2000658, NT2NE2000706, NT2NE2000802, NT2NE2000808, NT2NE2000864,

NT2NE2000913, NT2NE2000951, NT2NE2000980, NT2NE2001005, NT2NE2001021, NT2NE2001040, NT2NE2001049, NT2NE2001142, NT2NE2001156, NT2NE2001176, NT2NE2001180, NT2NE2001181, NT2NE2001188, NT2NE2001191, NT2NE2001247, NT2NE2001252, NT2NE2001324, NT2NE2001337, NT2NE2001364, NT2NE2001372, NT2NE2001397, NT2NE2001428, NT2NE2001432, NT2NE2001442, NT2NE2001524, NT2NE2001530, NT2NE2001598, NT2NE2001623, NT2NE2001626, NT2NE2001634, NT2NE2001666, NT2NE2001677, NT2NE2001697, NT2NE2001698, NT2NE2001723, NT2NE2001725, NT2NE2001874, NT2NE2001889, NT2NE2001923, NT2NE2002186, NT2NE2002285, NT2NE2002311, NT2NE2002620, NT2NE2002651, NT2NE2002732, NT2NE2002768, NT2NE2002791, NT2NE2002856, NT2NE2002870, NT2NE2003150,

NT2NE2003185, NT2NE2003252, NT2NE2003280, NT2NE2003308, NT2NE2003309, NT2NE2003315, NT2NE2003393, NT2NE2003408, NT2NE2003457, NT2NE2003540, NT2NE2003571, NT2NE2003705, NT2NE2003752, NT2NE2003921, NT2NE2004378, NT2NE2004490, NT2NE2004519, NT2NE2004606, NT2NE2004716, NT2NE2004787, NT2NE2004916, NT2NE2005035, NT2NE2005223, NT2NE2005276, NT2NE2005358, NT2NE2005395, NT2NE2005419, NT2NE2005441, NT2NE2005517, NT2NE2005537, NT2NE2005555, NT2NE2005588, NT2NE2005676, NT2NE2005720, NT2NE2005784, NT2NE2005804, NT2NE2005821, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006531,

NT2NE2006659, NT2NE2006784, NT2NE2006813, NT2NE2006894, NT2NE2006942, NT2NE2006958, NT2NE2007052, NT2NE2007220, NT2NE2007425, NT2NE2007725, NT2NE2007727, NT2NE2007877, NT2NE2007967, NT2NE2008017, NT2NE2008060, NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009295, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010854, NT2NE2011033, NT2NE2011036, NT2NE2011154, NT2NE2011221, NT2NE2011411, NT2NE2011485,

NT2NE2011650, NT2NE2011684, NT2NE2011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448, NT2NE2012457, NT2NE2012493, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014104, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015275, NT2NE2015480, NT2NE2015511, NT2NE2015565, NT2NE2015626, NT2NE2015747, NT2NE2015860, NT2NE2015885, NT2NE2015904, NT2NE2016041, NT2NE2016387,

NT2NE2016419, NT2NE2016519, NT2NE2016608, NT2NE2016766, NT2NE2016774, NT2NE2016971, NT2NE2017259, NT2NE2017332, NT2NE2017397, NT2NE2017480, NT2NE2017492, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018273, NT2NE2018376, NT2NE2018472, NT2NE2018490, NT2NE2018594, NT2NE2018739, NT2NE2018916, NT2NE2019056, NT2NE2019076, NT2NE2019099, NT2RI1000035, NT2RI1000164, NT2RI1000172, NT2RI2000004, NT2RI2000007, NT2RI2000117, NT2RI2000128, NT2RI2000133, NT2RI2000173, NT2RI2000187, NT2RI2000270, NT2RI2000276, NT2RI2000313, NT2RI2000341, NT2RI2000344, NT2RI2000421, NT2RI2000480, NT2RI2000575, NT2RI2000588,

NT2RI2000597, NT2RI2000669, NT2RI2000688, NT2RI2000689, NT2RI2000704, NT2RI2000738, NT2RI2000841, NT2RI2000865, NT2RI2000932, NT2RI2000987, NT2RI2001017, NT2RI2001030, NT2RI2001054, NT2RI2001083, NT2RI2001091, NT2RI2001167, NT2RI2001178, NT2RI2001230, NT2RI2001235, NT2RI2001244, NT2RI2001307, NT2RI2001385, NT2RI2001409, NT2RI2001410, NT2RI2001425, NT2RI2001449, NT2RI2001450, NT2RI2001474, NT2RI2001519, NT2RI2001595, NT2RI2001621, NT2RI2001624, NT2RI2001769, NT2RI2001782, NT2RI2001836, NT2RI2001952, NT2RI2002022, NT2RI2002041, NT2RI2002117, NT2RI2002120, NT2RI2002153, NT2RI2002179, NT2RI2002243, NT2RI2002252, NT2RI2002260, NT2RI2002266, NT2RI2002270, NT2RI2002334, NT2RI2002359, NT2RI2002447,

NT2RI2002481, NT2RI2002507, NT2RI2002517, NT2RI2002530, NT2RI2002549, NT2RI2002554, NT2RI2002564, NT2RI2002585, NT2RI2002592, NT2RI2002602, NT2RI2002699, NT2RI2002729, NT2RI2002802, NT2RI2002819, NT2RI2002822, NT2RI2002852, NT2RI2002923, NT2RI2002926, NT2RI2002970, NT2RI2003067, NT2RI2003184, NT2RI2003205, NT2RI2003222, NT2RI2003301, NT2RI2003360, NT2RI2003361, NT2RI2003383, NT2RI2003392, NT2RI2003399, NT2RI2003419, NT2RI2003481, NT2RI2003487, NT2RI2003556, NT2RI2003655, NT2RI2003667, NT2RI2003751, NT2RI2003884, NT2RI2003973, NT2RI2003993, NT2RI2004078, NT2RI2004079, NT2RI2004093, NT2RI2004136, NT2RI2004157, NT2RI2004188, NT2RI2004190, NT2RI2004209, NT2RI2004230, NT2RI2004284, NT2RI2004290,

NT2RI2004312, NT2RI2004381, NT2RI2004398, NT2RI2004410, NT2RI2004442, NT2RI2004468, NT2RI2004606, NT2RI2004618, NT2RI2004783, NT2RI2004818, NT2RI2004840, NT2RI2004916, NT2RI2004962, NT2RI2004984, NT2RI2004985, NT2RI2005048, NT2RI2005069, NT2RI2005087, NT2RI2005096, NT2RI2005115, NT2RI2005130, NT2RI2005166, NT2RI2005239, NT2RI2005246, NT2RI2005315, NT2RI2005358, NT2RI2005368, NT2RI2005382, NT2RI2005520, NT2RI2005564, NT2RI2005579, NT2RI2005621, NT2RI2005628, NT2RI2005670, NT2RI2005772, NT2RI2005816, NT2RI2005851, NT2RI2005966, NT2RI2006071, NT2RI2006081, NT2RI2006126, NT2RI2006127, NT2RI2006183, NT2RI2006257, NT2RI2006294, NT2RI2006345, NT2RI2006403, NT2RI2006445, NT2RI2006487, NT2RI2006506,

NT2RI2006553, NT2RI2006647, NT2RI2006659, NT2RI2006662, NT2RI2006667, NT2RI2006679, NT2RI2006686, NT2RI2006716, NT2RI2006735, NT2RI2006762, NT2RI2006788, NT2RI2006825, NT2RI2006838, NT2RI2006855, NT2RI2006973, NT2RI2007046, NT2RI2007048, NT2RI2007116, NT2RI2007148, NT2RI2007214, NT2RI2007303, NT2RI2007334, NT2RI2007386, NT2RI2007406, NT2RI2007439, NT2RI2007469, NT2RI2007498, NT2RI2007507, NT2RI2007593, NT2RI2007729, NT2RI2007751, NT2RI2007754, NT2RI2007827, NT2RI2007853, NT2RI2007877, NT2RI2007879, NT2RI2007891, NT2RI2007956, NT2RI2007965, NT2RI2008007, NT2RI2008050, NT2RI2008171, NT2RI2008188, NT2RI2008204, NT2RI2008221, NT2RI2008233, NT2RI2008455, NT2RI2008481, NT2RI2008502, NT2RI2008598,

NT2RI2008622, NT2RI2008656, NT2RI2008749, NT2RI2008801, NT2RI2008812, NT2RI2008841, NT2RI2008952, NT2RI2009005, NT2RI2009101, NT2RI2009103, NT2RI2009144, NT2RI2009151, NT2RI2009215, NT2RI2009233, NT2RI2009281, NT2RI2009289, NT2RI2009301, NT2RI2009402, NT2RI2009517, NT2RI2009585, NT2RI2009595, NT2RI2009855, NT2RI2010508, NT2RI2010795, NT2RI2011422, NT2RI2011683, NT2RI2011803, NT2RI2012350, NT2RI2012659, NT2RI2012990, NT2RI2013345, NT2RI2013357, NT2RI2013373, NT2RI2014247, NT2RI2014551, NT2RI2014683, NT2RI2014733, NT2RI2015239, NT2RI2016128, NT2RI2016134, NT2RI2017529, NT2RI2018311, NT2RI2018363, NT2RI2018883, NT2RI2018950, NT2RI2019751, NT2RI2019826, NT2RI2020390, NT2RI2020703, NT2RI2021463,

NT2RI2021625, NT2RI2022468, NT2RI2022584, NT2RI2023303, NT2RI2023671, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027323, NT2RI2027396, NT2RI2027484, NT2RI2027975, NT2RI2028213, NT2RI2028222, NT2RI2028537, NT2RI2028642, NT2RI3000213, NT2RI3000622, NT2RI3001132, NT2RI3001263, NT2RI3001279, NT2RI3001348, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003925, NT2RI3004133, NT2RI3004381,

NT2RI3004510, NT2RI3005202, NT2RI3005403, NT2RI3005416, NT2RI3005724, NT2RI3005861, NT2RI3005928, NT2RI3006132, NT2RI3006171, NT2RI3006340, NT2RI3006376, NT2RI3006666, NT2RI3006796, NT2RI3007065, NT2RI3007095, NT2RI3007158, NT2RI3007167, NT2RI3007213, NT2RI3007443, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009524, NT2RP6000005, NT2RP6000008, NT2RP6000032, NT2RP6000033, NT2RP6000043, NT2RP6000059, NT2RP6000060, NT2RP6000077, NT2RP6000086, NT2RP6000100, NT2RP6000109, NT2RP6000119, NT2RP6000127, NT2RP6000130, NT2RP6000140, NT2RP6000150, NT2RP7000041,

NT2RP7000109, NT2RP7000311, NT2RP7000359, NT2RP7000364, NT2RP7000380, NT2RP7000391, NT2RP7000425, NT2RP7000579, NT2RP7000586, NT2RP7000624, NT2RP7000812, NT2RP7000926, NT2RP7001074, NT2RP7001080, NT2RP7001156, NT2RP7001306, NT2RP7001319, NT2RP7001364, NT2RP7001475, NT2RP7001532, NT2RP7001602, NT2RP7001678, NT2RP7001856, NT2RP7002064, NT2RP7002151, NT2RP7002163, NT2RP7002243, NT2RP7002478, NT2RP7002554, NT2RP7002594, NT2RP7002603, NT2RP7002650, NT2RP7002738, NT2RP7002779, NT2RP7002841, NT2RP7002906, NT2RP7002978, NT2RP7002982, NT2RP7003025, NT2RP7003050, NT2RP7003055, NT2RP7003084, NT2RP7003091, NT2RP7003107, NT2RP7003134, NT2RP7003148, NT2RP7003261, NT2RP7003439, NT2RP7003680, NT2RP7003724,

NT2RP7004027, NT2RP7004080, NT2RP7004233, NT2RP7004358, NT2RP7004396, NT2RP7004481, NT2RP7004541, NT2RP7004559, NT2RP7004722, NT2RP7004766, NT2RP7004790, NT2RP7004915, NT2RP7004964, NT2RP7004975, NT2RP7005118, NT2RP7005205, NT2RP7005219, NT2RP7005323, NT2RP7005468, NT2RP7005513, NT2RP7005629, NT2RP7005631, NT2RP7005669, NT2RP7005846, NT2RP7006033, NT2RP7006075, NT2RP7006160, NT2RP7006162, NT2RP7006263, NT2RP7006296, NT2RP7006304, NT2RP7006374, NT2RP7006395, NT2RP7006408, NT2RP7006457, NT2RP7006547, NT2RP7006621, NT2RP7006636, NT2RP7006853, NT2RP7006887, NT2RP7006980, NT2RP7007114, NT2RP7007221, NT2RP7007226, NT2RP7007269, NT2RP7007310, NT2RP7007359, NT2RP7007480, NT2RP7007504, NT2RP7007594,

NT2RP7007617, NT2RP7007643, NT2RP7007842, NT2RP7007925, NT2RP7007930, NT2RP7008013, NT2RP7008133, NT2RP7008137, NT2RP7008142, NT2RP7008144, NT2RP7008190, NT2RP7008360, NT2RP7008396, NT2RP7008406, NT2RP7008435, NT2RP7008441, NT2RP7008454, NT2RP7008487, NT2RP7008544, NT2RP7008550, NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009168, NT2RP7009215, NT2RP7009225, NT2RP7009236, NT2RP7009253, NT2RP7009267, NT2RP7009322, NT2RP7009363, NT2RP7009370, NT2RP7009373, NT2RP7009397, NT2RP7009429, NT2RP7009439, NT2RP7009502, NT2RP7009507, NT2RP7009577, NT2RP7009743, NT2RP7009867, NT2RP7010128, NT2RP7010235,

NT2RP7010275, NT2RP7010521, NT2RP7010599, NT2RP7010612, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291, NT2RP7012516, NT2RP7012776, NT2RP7013002, NT2RP7013374, NT2RP7013499, NT2RP7013729, NT2RP7013999, NT2RP7014178, NT2RP7014348, NT2RP7014420, NT2RP7014721, NT2RP7014743, NT2RP7014778, NT2RP7014906, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017474, NT2RP7017546, NT2RP7017567, NT2RP7017795, NT2RP7017.971, NT2RP7018032, NT2RP7018126, NT2RP7018197,

NT2RP7018206, NT2RP7018340, NT2RP7018586, NT2RP7018802, NT2RP7018871, NT2RP7019064, NT2RP7019135, NT2RP7019273, NT2RP7019367, NT2RP7019401, NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8006452, NT2RP8006521, NT2RP8007416, NT2RP8007503, NT2RP8007715, NT2RP8009248, NTONG1000006, NTONG1000047, NTONG1000052, NTONG1000087, NTONG1000123, NTONG1000161, NTONG1000172, NTONG1000213, NTONG1000247, NTONG1000257, NTONG2000040, NTONG2000107,

NTONG2000231, NTONG2000334, NTONG2000363, NTONG2000413, NTONG2000492, NTONG2000499, NTONG2000531, NTONG2000583, NTONG2000858, NTONG2000878, NTONG2000966, NTONG2001014, NTONG2001079, NTONG2001157, NTONG2001173, NTONG2001220, NTONG2001222, NTONG2001362, NTONG2001428, NTONG2001532, NTONG2001587, NTONG2001619, NTONG2001642, NTONG2001657, NTONG2001676, NTONG2002278, NTONG2002807, NTONG2002948, NTONG2002970, NTONG2002985, NTONG2003158, NTONG2003210, NTONG2003316, NTONG2003409, NTONG2003515, NTONG2003805, NTONG2003839, NTONG2003852, NTONG2003897, NTONG2004521, NTONG2004690, NTONG2004806, NTONG2004829, NTONG2004844, NTONG2004991, NTONG2005062, NTONG2005086, NTONG2005137, NTONG2005153, NTONG2005265,

NTONG2005277, NTONG2005363, NTONG2005480, NTONG2005497, NTONG2005577, NTONG2005657, NTONG2005822, NTONG2005897, NTONG2006099, NTONG2006187, NTONG2006301, NTONG2006324, NTONG2006354, NTONG2006440, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008365, NTONG2008672, NTONG2008862, NTONG2008939, NTONG2009068, NTONG2009229, NTONG2009233, NTONG2009468, OCBBF1000016, OCBBF1000030, OCBBF1000042, OCBBF1000049, OCBBF1000054, OCBBF1000067, OCBBF1000085, OCBBF1000091, OCBBF1000114, OCBBF1000118, OCBBF1000119, OCBBF1000122, OCBBF1000130,

OCBBF1000138, OCBBF1000145, OCBBF1000153, OCBBF1000175, OCBBF1000185, OCBBF1000188, OCBBF1000192, OCBBF1000254, OCBBF2000277, OCBBF2000287, OCBBF2000323, OCBBF2000452, OCBBF2000467, OCBBF2000703, OCBBF2000712, OCBBF2000719, OCBBF2000824, OCBBF2000904, OCBBF2000998, OCBBF2001075, OCBBF2001101, OCBBF2001124, OCBBF2001140, OCBBF2001176, OCBBF2001186, OCBBF2001210, OCBBF2001252, OCBBF2001323, OCBBF2001389, OCBBF2001402, OCBBF2001408, OCBBF2001416, OCBBF2001473, OCBBF2001494, OCBBF2001527, OCBBF2001586, OCBBF2001681, OCBBF2001749, OCBBF2001794, OCBBF2001910, OCBBF2001931, OCBBF2001961, OCBBF2002083, OCBBF2002124, OCBBF2002161, OCBBF2002357, OCBBF2002376, OCBBF2002615, OCBBF2002626, OCBBF2002663,

OCBBF2002805, OCBBF2002865, OCBBF2002920, OCBBF2002980, OCBBF2003028, OCBBF2003091, OCBBF2003246, OCBBF2003327, OCBBF2003593, OCBBF2004038, OCBBF2004147, OCBBF2004168, OCBBF2004235, OCBBF2004259, OCBBF2004385, OCBBF2004418, OCBBF2004478, OCBBF2004482, OCBBF2004533, OCBBF2004567, OCBBF2004612, OCBBF2004647, OCBBF2004669, OCBBF2004757, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005476, OCBBF2005546, OCBBF2005843, OCBBF2005901, OCBBF2005956, OCBBF2006005, OCBBF2006030, OCBBF2006058, OCBBF2006113, OCBBF2006148, OCBBF2006151,

OCBBF2006154, OCBBF2006172, OCBBF2006214, OCBBF2006241, OCBBF2006313, OCBBF2006388, OCBBF2006438, OCBBF2006567, OCBBF2006624, OCBBF2006639, OCBBF2006764, OCBBF2006859, OCBBF2006972, OCBBF2006987, OCBBF2007039, OCBBF2007051, OCBBF2007068, OCBBF2007114, OCBBF2007184, OCBBF2007194, OCBBF2007196, OCBBF2007238, OCBBF2007428, OCBBF2007478, OCBBF2007485, OCBBF2007520, OCBBF2007555, OCBBF2007610, OCBBF2007622, OCBBF2007756, OCBBF2007762, OCBBF2007931, OCBBF2007946, OCBBF2008116, OCBBF2008138, OCBBF2008144, OCBBF2008283, OCBBF2008330, OCBBF2008340, OCBBF2008366, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724,

OCBBF2008760, OCBBF2008768, OCBBF2008770, OCBBF2008775, OCBBF2008790, OCBBF2008814, OCBBF2009095, OCBBF2009115, OCBBF2009352, OCBBF2009391, OCBBF2009424, OCBBF2009536, OCBBF2009571, OCBBF2009583, OCBBF2009603, OCBBF2009788, OCBBF2009920, OCBBF2009926, OCBBF2010040, OCBBF2010313, OCBBF2010404, OCBBF2010416, OCBBF2010420, OCBBF2010604, OCBBF2010709, OCBBF2010792, OCBBF2010819, OCBBF2010843, OCBBF2010858, OCBBF2010863, OCBBF2010871, OCBBF2010931, OCBBF2010945, OCBBF2010978, OCBBF2010989, OCBBF2011073, OCBBF2011137, OCBBF2011160, OCBBF2011177, OCBBF2011228, OCBBF2011232, OCBBF2011283, OCBBF2011311, OCBBF2011536, OCBBF2011625, OCBBF2011669, OCBBF2011685, OCBBF2011706, OCBBF2011722, OCBBF2011855,

OCBBF2011872, OCBBF2011897, OCBBF2011914, OCBBF2011960, OCBBF2011981, OCBBF2012028, OCBBF2012039, OCBBF2012095, OCBBF2012139, OCBBF2012191, OCBBF2012262, OCBBF2012320, OCBBF2012525, OCBBF2012678, OCBBF2012704, OCBBF2012755, OCBBF2012812, OCBBF2012881, OCBBF2012908, OCBBF2012936, OCBBF2012990, OCBBF2013049, OCBBF2013091, OCBBF2013127, OCBBF2013208, OCBBF2013285, OCBBF2013319, OCBBF2013324, OCBBF2013481, OCBBF2013507, OCBBF2013721, OCBBF2013789, OCBBF2013883, OCBBF2013926, OCBBF2014089, OCBBF2014292, OCBBF2014322, OCBBF2014386, OCBBF2014541, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232,

OCBBF2015233, OCBBF2015285, OCBBF2015334, OCBBF2015335, OCBBF2015503, OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016689, OCBBF2016690, OCBBF2016841, OCBBF2016928, OCBBF2017069, OCBBF2017306, OCBBF2017325, OCBBF2017458, OCBBF2017489, OCBBF2017516, OCBBF2017665, OCBBF2017815, OCBBF2017882, OCBBF2017888, OCBBF2017920, OCBBF2018014, OCBBF2018084, OCBBF2018167, OCBBF2018206, OCBBF2018229, OCBBF2018234, OCBBF2018362, OCBBF2018380, OCBBF2018618, OCBBF2018663, OCBBF2018687, OCBBF2018707, OCBBF2018828, OCBBF2018873, OCBBF2018934, OCBBF2019195, OCBBF2019327,

OCBBF2019684, OCBBF2019761, OCBBF2019823, OCBBF2019950, OCBBF2020048, OCBBF2020343, OCBBF2020453, OCBBF2020741, OCBBF2020825, OCBBF2020838, OCBBF2021020, OCBBF2021214, OCBBF2021286, OCBBF2021323, OCBBF2021518, OCBBF2021788, OCBBF2021833, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026368, OCBBF2026645, OCBBF2026649, OCBBF2026690, OCBBF2026981, OCBBF2027148, OCBBF2027197, OCBBF2027354, OCBBF2027423, OCBBF2027478, OCBBF2027481,

OCBBF2027511, OCBBF2027661, OCBBF2027728, OCBBF2027782, OCBBF2027868, OCBBF2028055, OCBBF2028173, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035226, OCBBF2035390, OCBBF2035564, OCBBF2035658, OCBBF2035885, OCBBF2035916, OCBBF2036019, OCBBF2036031, OCBBF2036225, OCBBF2036476, OCBBF2036752, OCBBF2037068, OCBBF2037340,

OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000185, PEBLM1000024, PEBLM1000029, PEBLM1000034, PEBLM1000062,

PEBLM1000068, PEBLM1000071, PEBLM1000083, PEBLM1000147, PEBLM1000174, PEBLM1000180, PEBLM2000112, PEBLM2000126, PEBLM2000170, PEBLM2000180, PEBLM2000213, PEBLM2000248, PEBLM2000267, PEBLM2000270, PEBLM2000321, PEBLM2000333, PEBLM2000479, PEBLM2000502, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003935, PEBLM2004015, PEBLM2004216, PEBLM2004290, PEBLM2004497, PEBLM2004733, PEBLM2005282, PEBLM2005615, PEBLM2005632, PEBLM2005697, PEBLM2006031, PEBLM2006049, PEBLM2006113, PEBLM2006135, PEBLM2006215, PEBLM2006283, PEBLM2006298, PEBLM2006366, PEBLM2006912, PEBLM2007112, PEBLM2007140,

PEBLM2007437, PEBLM2007598, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287, PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117,

PERIC2005305, PERIC2005347, PERIC2005370, PERIC2005936, PERIC2006035, PERIC2006121, PERIC2006443, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000037, PLACE5000058, PLACE5000066, PLACE5000067, PLACE5000070, PLACE5000080, PLACE5000105, PLACE5000115, PLACE5000116, PLACE5000129, PLACE5000139, PLACE5000153, PLACE5000171, PLACE5000172, PLACE5000178, PLACE5000245, PLACE5000260, PLACE5000282, PLACE5000297, PLACE5000372, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000263,

PLACE6000296, PLACE6000314, PLACE6000348, PLACE6000371, PLACE6000424, PLACE6000426, PLACE6000429, PLACE6000450, PLACE6000523, PLACE6000550, PLACE6000552, PLACE6000555, PLACE6000619, PLACE6000953, PLACE6001118, PLACE6001153, PLACE6001185, PLACE6001262, PLACE6001294, PLACE6001536, PLACE6001557, PLACE6001712, PLACE6001740, PLACE6001823, PLACE6001886, PLACE6001923, PLACE6001925, PLACE6001933, PLACE6002011, PLACE6002016, PLACE6002056, PLACE6002157, PLACE6002312, PLACE6002323, PLACE6002345, PLACE6002419, PLACE6002462, PLACE6002647, PLACE6002692, PLACE6002699, PLACE6002710, PLACE6002871, PLACE6002949, PLACE6003004, PLACE6003038, PLACE6003077, PLACE6003094, PLACE6003109, PLACE6003204, PLACE6003372,

PLACE6003393, PLACE6003399, PLACE6003539, PLACE6003621, PLACE6003705, PLACE6003740, PLACE6003771, PLACE6003796, PLACE6003839, PLACE6003850, PLACE6004101, PLACE6004219, PLACE6004336, PLACE6004368, PLACE6004380, PLACE6004396, PLACE6004454, PLACE6004464, PLACE6004491, PLACE6004578, PLACE6004663, PLACE6004687, PLACE6004738, PLACE6004931, PLACE6004966, PLACE6004993, PLACE6005007, PLACE6005029, PLACE6005108, PLACE6005234, PLACE6005283, PLACE6005328, PLACE6005351, PLACE6005423, PLACE6005487, PLACE6005535, PLACE6005546, PLACE6005559, PLACE6005579, PLACE6005691, PLACE6005786, PLACE6006077, PLACE6006158, PLACE6006217, PLACE6006222, PLACE6006266, PLACE6006287, PLACE6006394, PLACE6006418, PLACE6006474,

PLACE6006566, PLACE6006697, PLACE6006822, PLACE6006871, PLACE6006905, PLACE6006988, PLACE6007050, PLACE6007180, PLACE6007242, PLACE6007309, PLACE6007373, PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008824, PLACE6008989, PLACE6009006, PLACE6009008, PLACE6009228, PLACE6009338, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010485, PLACE6010568, PLACE6010701, PLACE6010704,

PLACE6010732, PLACE6010765, PLACE6010925, PLACE6010936, PLACE6010991, PLACE6011041, PLACE6011102, PLACE6011156, PLACE6011211, PLACE6011260, PLACE6011334, PLACE6011627, PLACE6011774, PLACE6011881, PLACE6011907, PLACE6011975, PLACE6012108, PLACE6012168, PLACE6012297, PLACE6012308, PLACE6012574, PLACE6012908, PLACE6012942, PLACE6013288, PLACE6013386, PLACE6013400, PLACE6013650, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015731, PLACE6015939, PLACE6016030, PLACE6016093, PLACE6016105, PLACE6016160, PLACE6016210, PLACE6016383,

PLACE6017002, PLACE6017431, PLACE6017564, PLACE6017578, PLACE6017626, PLACE6017701, PLACE6017714, PLACE6017788, PLACE6017791, PLACE6018107, PLACE6018187, PLACE6018235, PLACE6018287, PLACE6018441, PLACE6018497, PLACE6018769, PLACE6018834, PLACE6018843, PLACE6018863, PLACE6019385, PLACE6019542, PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001544, PLACE7001759, PLACE7001936, PLACE7002303, PLACE7002641, PLACE7003639, PLACE7003657, PLACE7003684, PLACE7003985, PLACE7004961, PLACE7005169, PLACE7005671, PLACE7006051, PLACE7006090, PLACE7006240,

PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000110, PROST1000123, PROST1000136, PROST1000144, PROST1000145, PROST1000152, PROST1000167, PROST1000184, PROST1000217, PROST1000262, PROST1000272, PROST1000298, PROST1000322, PROST1000334, PROST1000362, PROST1000377, PROST1000383, PROST1000412, PROST1000419, PROST1000451, PROST1000480, PROST1000485, PROST1000493, PROST1000512, PROST1000527, PROST1000528, PROST1000559, PROST1000575, PROST2000053, PROST2000067, PROST2000079, PROST2000105,

PROST2000143, PROST2000162, PROST2000165, PROST2000176, PROST2000203, PROST2000206, PROST2000212, PROST2000267, PROST2000273, PROST2000325, PROST2000337, PROST2000432, PROST2000452, PROST2000463, PROST2000470, PROST2000505, PROST2000572, PROST2000717, PROST2000761, PROST2000893, PROST2000961, PROST2001138, PROST2001213, PROST2001283, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001823, PROST2001899, PROST2001998, PROST2002078, PROST2002101, PROST2002162, PROST2002212, PROST2002227, PROST2002338, PROST2002425, PROST2002488, PROST2002489, PROST2002527, PROST2002591,

PROST2002602, PROST2002633, PROST2002682, PROST2002685, PROST2002722, PROST2002736, PROST2002842, PROST2002906, PROST2002927, PROST2003102, PROST2003117, PROST2003210, PROST2003302, PROST2003303, PROST2003324, PROST2003340, PROST2003428, PROST2003472, PROST2003563, PROST2003577, PROST2003583, PROST2003586, PROST2003628, PROST2003635, PROST2003674, PROST2003676, PROST2003732, PROST2003775, PROST2003922, PROST2003930, PROST2004115, PROST2004142, PROST2004146, PROST2004161, PROST2004251, PROST2004258, PROST2004332, PROST2004481, PROST2004526, PROST2004570, PROST2004727, PROST2004742, PROST2004817, PROST2005039, PROST2005076, PROST2005093, PROST2005121, PROST2005131, PROST2005228, PROST2005426,

PROST2005466, PROST2005630, PROST2005683, PROST2005788, PROST2005880, PROST2005886, PROST2005904, PROST2005919, PROST2005943, PROST2006008, PROST2006020, PROST2006196, PROST2006201, PROST2006282, PROST2006450, PROST2006491, PROST2006510, PROST2006579, PROST2006688, PROST2006766, PROST2006782, PROST2006933, PROST2006988, PROST2007122, PROST2007200, PROST2007237, PROST2007248, PROST2007289, PROST2007317, PROST2007328, PROST2007382, PROST2007389, PROST2007416, PROST2007528, PROST2007818, PROST2007871, PROST2007950, PROST2007956, PROST2007974, PROST2007988, PROST2008079, PROST2008088, PROST2008243, PROST2008245, PROST2008268, PROST2008447, PROST2008468, PROST2008472, PROST2008516, PROST2008724,

PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010250, PROST2010318, PROST2010326, PROST2010382, PROST2010400, PROST2010606, PROST2010885, PROST2011012, PROST2011038, PROST2011105, PROST2011297, PROST2011410, PROST2011439, PROST2011482, PROST2011631, PROST2011660, PROST2012005, PROST2012016, PROST2012088, PROST2012140, PROST2012157, PROST2012190, PROST2012249, PROST2012353, PROST2012400, PROST2012542, PROST2012550, PROST2012740, PROST2012780,

PROST2012888, PROST2012890, PROST2013032, PROST2013053, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014916, PROST2014925, PROST2015124, PROST2015137, PROST2015198, PROST2015243, PROST2015246, PROST2015251, PROST2015332, PROST2015457, PROST2015464, PROST2015537, PROST2015545, PROST2015551, PROST2015636, PROST2015710, PROST2015756, PROST2015877, PROST2015900, PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829,

PROST2016860, PROST2016918, PROST2016980, PROST2017098, PROST2017367, PROST2017413, PROST2017441, PROST2017529, PROST2017578, PROST2017617, PROST2017692, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2018977, PROST2019296, PROST2019398, PROST2019487, PROST2019781, PUAEN1000039, PUAEN1000081, PUAEN1000085, PUAEN1000087, PUAEN1000121, PUAEN1000175, PUAEN1000239, PUAEN1000275, PUAEN1000276, PUAEN1000288, PUAEN1000322, PUAEN2000080, PUAEN2000175, PUAEN2000247, PUAEN2000394, PUAEN2000497,

PUAEN2000594, PUAEN2000684, PUAEN2000942, PUAEN2001260, PUAEN2001345, PUAEN2001526, PUAEN2001882, PUAEN2002473, PUAEN2002489, PUAEN2002552, PUAEN2002568, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004511, PUAEN2004525, PUAEN2004875, PUAEN2005110, PUAEN2005247, PUAEN2005502, PUAEN2005588, PUAEN2006029, PUAEN2006335, PUAEN2006578, PUAEN2006639, PUAEN2006761, PUAEN2007350, PUAEN2007785, PUAEN2007898, PUAEN2008123, PUAEN2008228, PUAEN2008314, PUAEN2008515, PUAEN2008815, PUAEN2008980, PUAEN2009348, PUAEN2009534, PUAEN2009655, PUAEN2009795, PUAEN2009852, PUAEN2010824, RECTM1000051, RECTM1000141,

RECTM2000220, RECTM2000349, RECTM2000433, RECTM2000510, RECTM2001307, RECTM2001347, RECTM2001519, RECTM2001666, RECTM2001691, RECTM2001749, RECTM2002602, RECTM2002632, SALGL1000005, SALGL1000024, SALGL1000028, SALGL1000065, SALGL1000107, SKMUS1000030, SKMUS1000064, SKMUS1000067, SKMUS1000102, SKMUS1000104, SKMUS1000118, SKMUS1000124, SKMUS1000129, SKMUS1000138, SKMUS1000176, SKMUS2000020, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000361, SKMUS2000365, SKMUS2000380, SKMUS2000390, SKMUS2000416, SKMUS2000458, SKMUS2000467, SKMUS2000468, SKMUS2000515, SKMUS2000690,

SKMUS2000701, SKMUS2000705, SKMUS2000710, SKMUS2000724, SKMUS2000726, SKMUS2000774, SKMUS2000780, SKMUS2000821, SKMUS2000847, SKMUS2000863, SKMUS2000873, SKMUS2000894, SKMUS2000898, SKMUS2000902, SKMUS2000945, SKMUS2000961, SKMUS2000973, SKMUS2000980, SKMUS2001008, SKMUS2001014, SKMUS2001129, SKMUS2001147, SKMUS2001157, SKMUS2001164, SKMUS2001199, SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001501, SKMUS2001543, SKMUS2001580, SKMUS2001585, SKMUS2001608, SKMUS2001631, SKMUS2001662, SKMUS2001663, SKMUS2001668, SKMUS2001671, SKMUS2001683, SKMUS2001693, SKMUS2001695,

SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667, SKMUS2004897, SKMUS2004903, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000110, SKNMC1000123, SKNMC1000124, SKNMC1000159, SKNMC2000256,

SKNMC2000305, SKNMC2000322, SKNMC2000374, SKNMC2000583, SKNMC2000593, SKNMC2000612, SKNMC2000622, SKNMC2000635, SKNMC2000966, SKNMC2001113, SKNMC2001324, SKNMC2001503, SKNMC2001555, SKNMC2001596, SKNMC2001623, SKNMC2002402, SKNMC2003639, SKNMC2003924, SKNMC2003987, SKNMC2004457, SKNMC2004643, SKNMC2004651, SKNMC2005772, SKNMC2006327, SKNMC2006998, SKNMC2007134, SKNMC2007502, SKNMC2007504, SKNMC2007961, SKNMC2009450, SKNSH1000086, SKNSH1000174, SKNSH1000301, SKNSH1000308, SKNSH1000416, SKNSH2000051, SKNSH2000151, SKNSH2000163, SKNSH2000319, SKNSH2000482, SKNSH2000516, SKNSH2000550, SKNSH2000716, SKNSH2000971, SKNSH2001875, SKNSH2001931, SKNSH2002054, SKNSH2002866, SKNSH2003064, SKNSH2003174,

SKNSH2003422, SKNSH2003466, SKNSH2003483, SKNSH2003633, SKNSH2004039, SKNSH2005061, SKNSH2005120, SKNSH2005194, SKNSH2005240, SKNSH2005792, SKNSH2005816, SKNSH2005981, SKNSH2006304, SKNSH2006432, SKNSH2006822, SKNSH2007142, SKNSH2007149, SKNSH2007429, SKNSH2007739, SKNSH2008043, SKNSH2008940, SKNSH2008969, SKNSH2009197, SKNSH2009357, SKNSH2009435, SKNSH2009991, SKNSH2010015, SMINT1000012, SMINT1000039, SMINT1000048, SMINT1000051, SMINT1000054, SMINT1000057, SMINT1000071, SMINT1000075, SMINT1000078, SMINT1000091, SMINT1000100, SMINT1000103, SMINT1000118, SMINT1000131, SMINT1000137, SMINT2000018, SMINT2000032, SMINT2000040, SMINT2000060, SMINT2000073, SMINT2000084, SMINT2000145, SMINT2000176,

SMINT2000185, SMINT2000227, SMINT2000229, SMINT2000267, SMINT2000400, SMINT2000518, SMINT2000529, SMINT2000530, SMINT2000537, SMINT2000538, SMINT2000541, SMINT2000558, SMINT2000562, SMINT2000568, SMINT2000609, SMINT2000811, SMINT2000824, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001950, SMINT2002126, SMINT2002202, SMINT2002210, SMINT2002281, SMINT2002311, SMINT2002314, SMINT2002328, SMINT2002414, SMINT2002457, SMINT2002743, SMINT2002778, SMINT2002880, SMINT2002884, SMINT2002899, SMINT2002974,

SMINT2003128, SMINT2003150, SMINT2003317, SMINT2003386, SMINT2003551, SMINT2003569, SMINT2003641, SMINT2003644, SMINT2003866, SMINT2004047, SMINT2004086, SMINT2004299, SMINT2004339, SMINT2004350, SMINT2004414, SMINT2004473, SMINT2004547, SMINT2004583, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005368, SMINT2005402, SMINT2005405, SMINT2005621, SMINT2005623, SMINT2005624, SMINT2005688, SMINT2005800, SMINT2006078, SMINT2006205, SMINT2006331, SMINT2006596, SMINT2006641, SMINT2006648, SMINT2006708, SMINT2006716, SMINT2007101, SMINT2007140, SMINT2007219, SMINT2007294,

SMINT2007365, SMINT2007391, SMINT2007433, SMINT2007508, SMINT2007526, SMINT2007579, SMINT2007647, SMINT2007729, SMINT2007790, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2009119, SMINT2009121, SMINT2009212, SMINT2009216, SMINT2009233, SMINT2009259, SMINT2009272, SMINT2009363, SMINT2009415, SMINT2009468, SMINT2009505, SMINT2009529, SMINT2009832, SMINT2009895, SMINT2009902, SMINT2009973, SMINT2010015, SMINT2010068,

SMINT2010076, SMINT2010144, SMINT2010200, SMINT2010278, SMINT2010324, SMINT2010326, SMINT2010426, SMINT2010500, SMINT2010533, SMINT2010629, SMINT2010639, SMINT2010672, SMINT2010753, SMINT2010853, SMINT2010897, SMINT2010959, SMINT2010997, SMINT2011033, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012122, SMINT2012195, SMINT2012285, SMINT2012501, SMINT2012734, SMINT2012793, SMINT2013129, SMINT2013170, SMINT2013181, SMINT2013228, SMINT2013613, SMINT2013626, SMINT2013695, SMINT2013833, SMINT2013890, SMINT2014166, SMINT2014282, SMINT2014443, SMINT2014480, SMINT2014489, SMINT2014721,

SMINT2015006, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017974, SMINT2018343, SMINT2018353, SMINT2018592, SMINT2018681, SMINT2019017, SMINT2019105, SMINT2019142, SMINT2019153, SMINT2019200, SPLEN1000013, SPLEN1000049, SPLEN1000087, SPLEN1000089, SPLEN1000091, SPLEN1000139, SPLEN1000143, SPLEN1000144,

SPLEN1000145, SPLEN1000156, SPLEN1000163, SPLEN1000165, SPLEN1000178, SPLEN2000020, SPLEN2000064, SPLEN2000126, SPLEN2000134, SPLEN2000189, SPLEN2000197, SPLEN2000205, SPLEN2000210, SPLEN2000222, SPLEN2000242, SPLEN2000247, SPLEN2000258, SPLEN2000270, SPLEN2000307, SPLEN2000310, SPLEN2000341, SPLEN2000348, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464, SPLEN2000496, SPLEN2000515, SPLEN2000537, SPLEN2000541, SPLEN2000607, SPLEN2000630, SPLEN2000695, SPLEN2000698, SPLEN2000839, SPLEN2001135, SPLEN2001176, SPLEN2001227, SPLEN2001245, SPLEN2001354, SPLEN2001367, SPLEN2001383, SPLEN2001503, SPLEN2001510,

SPLEN2001599, SPLEN2001626, SPLEN2001650, SPLEN2001689, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002272, SPLEN2002314, SPLEN2002343, SPLEN2002354, SPLEN2002385, SPLEN2002419, SPLEN2002462, SPLEN2002467, SPLEN2002477, SPLEN2002662, SPLEN2002695, SPLEN2002707, SPLEN2002917, SPLEN2002957, SPLEN2003160, SPLEN2003204, SPLEN2003219, SPLEN2003396, SPLEN2003678, SPLEN2003878, SPLEN2003918, SPLEN2003924, SPLEN2004060, SPLEN2004078, SPLEN2004124, SPLEN2004148, SPLEN2004343, SPLEN2004346, SPLEN2004368, SPLEN2004844, SPLEN2004880, SPLEN2005009, SPLEN2005227, SPLEN2005272, SPLEN2005450,

SPLEN2005474, SPLEN2005727, SPLEN2005838, SPLEN2005869, SPLEN2005927, SPLEN2005939, SPLEN2006268, SPLEN2006283, SPLEN2006325, SPLEN2006389, SPLEN2006425, SPLEN2006671, SPLEN2006701, SPLEN2006785, SPLEN2006875, SPLEN2007276, SPLEN2007314, SPLEN2007350, SPLEN2007619, SPLEN2007653, SPLEN2007689, SPLEN2007695, SPLEN2007750, SPLEN2007926, SPLEN2007951, SPLEN2008007, SPLEN2008135, SPLEN2008164, SPLEN2008317, SPLEN2008496, SPLEN2008591, SPLEN2008786, SPLEN2009088, SPLEN2009315, SPLEN2009340, SPLEN2009541, SPLEN2009548, SPLEN2009555, SPLEN2009581, SPLEN2009733, SPLEN2009803, SPLEN2009884, SPLEN2009918, SPLEN2010004, SPLEN2010112, SPLEN2010119, SPLEN2010146, SPLEN2010187, SPLEN2010350, SPLEN2010395,

SPLEN2010415, SPLEN2010447, SPLEN2010469, SPLEN2010510, SPLEN2010530, SPLEN2010534, SPLEN2010588, SPLEN2010800, SPLEN2010847, SPLEN2010862, SPLEN2010978, SPLEN2010986, SPLEN2011018, SPLEN2011025, SPLEN2011086, SPLEN2011139, SPLEN2011145, SPLEN2011248, SPLEN2011252, SPLEN2011419, SPLEN2011422, SPLEN2011672, SPLEN2011678, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012524, SPLEN2012525, SPLEN2012606, SPLEN2012611, SPLEN2012619, SPLEN2012725, SPLEN2012743, SPLEN2012756, SPLEN2012961, SPLEN2013195, SPLEN2013503, SPLEN2013644, SPLEN2013648, SPLEN2013673, SPLEN2013690, SPLEN2013714,

SPLEN2013821, SPLEN2013857, SPLEN2013860, SPLEN2013910, SPLEN2013936, SPLEN2014063, SPLEN2014073, SPLEN2014119, SPLEN2014136, SPLEN2014199, SPLEN2014210, SPLEN2014293, SPLEN2014318, SPLEN2014381, SPLEN2014454, SPLEN2014486, SPLEN2014543, SPLEN2014572, SPLEN2014645, SPLEN2014669, SPLEN2014711, SPLEN2014860, SPLEN2014865, SPLEN2014919, SPLEN2014920, SPLEN2014924, SPLEN2014937, SPLEN2014946, SPLEN2015031, SPLEN2015094, SPLEN2015129, SPLEN2015158, SPLEN2015166, SPLEN2015216, SPLEN2015258, SPLEN2015261, SPLEN2015267, SPLEN2015276, SPLEN2015415, SPLEN2015433, SPLEN2015593, SPLEN2015674, SPLEN2015679, SPLEN2015707, SPLEN2015730, SPLEN2015746, SPLEN2015815, SPLEN2015890, SPLEN2015891, SPLEN2015899,

SPLEN2016045, SPLEN2016053, SPLEN2016069, SPLEN2016135, SPLEN2016241, SPLEN2016250, SPLEN2016304, SPLEN2016356, SPLEN2016480, SPLEN2016531, SPLEN2016533, SPLEN2016541, SPLEN2016552, SPLEN2016554, SPLEN2016602, SPLEN2016627, SPLEN2016773, SPLEN2016840, SPLEN2016863, SPLEN2016922, SPLEN2016932, SPLEN2016972, SPLEN2016973, SPLEN2017104, SPLEN2017121, SPLEN2017147, SPLEN2017187, SPLEN2017189, SPLEN2017212, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017918, SPLEN2017999, SPLEN2018098, SPLEN2018112, SPLEN2018159, SPLEN2018180, SPLEN2018181, SPLEN2018280, SPLEN2018285, SPLEN2018302, SPLEN2018364,

SPLEN2018395, SPLEN2018643, SPLEN2018732, SPLEN2018749, SPLEN2018933, SPLEN2019008, SPLEN2019043, SPLEN2019092, SPLEN2019102, SPLEN2019131, SPLEN2019257, SPLEN2019311, SPLEN2019323, SPLEN2019349, SPLEN2019375, SPLEN2019379, SPLEN2019471, SPLEN2019480, SPLEN2019571, SPLEN2019575, SPLEN2019709, SPLEN2019774, SPLEN2019793, SPLEN2019811, SPLEN2019839, SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021231, SPLEN2021273, SPLEN2021295, SPLEN2021298, SPLEN2021383, SPLEN2021417,

SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022591, SPLEN2022785, SPLEN2022920, SPLEN2023024, SPLEN2023160, SPLEN2023423, SPLEN2023710, SPLEN2023791, SPLEN2023977, SPLEN2024070, SPLEN2024127, SPLEN2024273, SPLEN2024383, SPLEN2024530, SPLEN2024571, SPLEN2024577, SPLEN2024617, SPLEN2024737, SPLEN2024956, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138,

SPLEN2028365, SPLEN2028417, SPLEN2028424, SPLEN2028593, SPLEN2028844, SPLEN2028994, SPLEN2029051, SPLEN2029176, SPLEN2029380, SPLEN2029522, SPLEN2029727, SPLEN2029904, SPLEN2030250, SPLEN2030335, SPLEN2030371, SPLEN2030397, SPLEN2030479, SPLEN2030495, SPLEN2030562, SPLEN2030763, SPLEN2030847, SPLEN2031004, SPLEN2031125, SPLEN2031424, SPLEN2031724, SPLEN2031780, SPLEN2032036, SPLEN2032112, SPLEN2032154, SPLEN2032321, SPLEN2032356, SPLEN2032677, SPLEN2032813, SPLEN2032924, SPLEN2033153, SPLEN2033333, SPLEN2033490, SPLEN2033539, SPLEN2033921, SPLEN2033996, SPLEN2034021, SPLEN2034081, SPLEN2034252, SPLEN2034551, SPLEN2034601, SPLEN2034678, SPLEN2034864, SPLEN2034934, SPLEN2035318, SPLEN2035615,

SPLEN2036103, SPLEN2036326, SPLEN2036475, SPLEN2036579, SPLEN2036608, SPLEN2036702, SPLEN2036712, SPLEN2036821, SPLEN2036932, SPLEN2036953, SPLEN2037077, SPLEN2037194, SPLEN2037319, SPLEN2037444, SPLEN2037580, SPLEN2037630, SPLEN2037678, SPLEN2037891, SPLEN2038055, SPLEN2038180, SPLEN2038345, SPLEN2038407, SPLEN2038473, SPLEN2039311, SPLEN2039697, SPLEN2039845, SPLEN2039936, SPLEN2040222, SPLEN2040237, SPLEN2040824, SPLEN2041304, SPLEN2041310, SPLEN2041585, SPLEN2041645, SPLEN2041700, SPLEN2041720, SPLEN2041977, SPLEN2042051, SPLEN2042521, SPLEN2042598, SPLEN2042714, SPLEN2042920, STOMA1000013, STOMA1000052, STOMA1000074, STOMA1000091, STOMA1000117, STOMA1000133, STOMA1000175, STOMA1000186,

STOMA1000190, STOMA1000191, STOMA2000032, STOMA2000072, STOMA2000088, STOMA2000104, STOMA2000148, STOMA2000168, STOMA2000183, STOMA2000188, STOMA2000279, STOMA2000289, STOMA2000383, STOMA2000386, STOMA2000396, STOMA2000478, STOMA2000482, STOMA2000529, STOMA2000539, STOMA2000567, STOMA2000640, STOMA2000678, STOMA2000686, STOMA2000888, STOMA2001025, STOMA2001389, STOMA2002141, STOMA2002367, STOMA2002688, STOMA2003289, STOMA2003444, STOMA2003477, STOMA2003646, STOMA2003894, STOMA2004194, STOMA2004294, STOMA2004663, STOMA2004668, STOMA2004852, STOMA2004873, STOMA2004884, STOMA2004893, STOMA2004925, STOMA2005120, STOMA2005664, STOMA2005667, STOMA2005746, STOMA2005782, STOMA2006181, STOMA2006213,

STOMA2006229, STOMA2006325, STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, SYNOV1000045, SYNOV1000049, SYNOV1000118, SYNOV1000128, SYNOV1000190, SYNOV1000256, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001144, SYNOV2001212, SYNOV2001251, SYNOV2001300,

SYNOV2001356, SYNOV2001390, SYNOV2001451, SYNOV2001457, SYNOV2001468, SYNOV2001660, SYNOV2003326, SYNOV2005216, SYNOV2005448, SYNOV2005673, SYNOV2005817, SYNOV2005962, SYNOV2006430, SYNOV2006620, SYNOV2007965, SYNOV2008765, SYNOV2011233, SYNOV2012326, SYNOV2013365, SYNOV2013637, SYNOV2014157, SYNOV2014364, SYNOV2016124, SYNOV2016837, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000598, SYNOV4000706, SYNOV4001224, SYNOV4001342, SYNOV4001395, SYNOV4002001, SYNOV4002392, SYNOV4002875, SYNOV4002883, SYNOV4003174, SYNOV4003322, SYNOV4003681, SYNOV4003981,

SYNOV4004184, SYNOV4004741, SYNOV4004914, SYNOV4005256, SYNOV4005570, SYNOV4005739, SYNOV4005889, SYNOV4005981, SYNOV4006256, SYNOV4007215, SYNOV4007360, SYNOV4007430, SYNOV4007521, SYNOV4007711, SYNOV4008336, SYNOV4009129, SYNOV4009139, SYNOV4009298, SYNOV4009575, T1ESE2002665, TBAES2000004, TBAES2000059, TBAES2000116, TBAES2000255, TBAES2000315, TBAES2000352, TBAES2000932, TBAES2001171, TBAES2001220, TBAES2001229, TBAES2001258, TBAES2001492, TBAES2001751, TBAES2003435, TBAES2003492, TBAES2003550, TBAES2003702, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504,

TBAES2007548, TBAES2007697, TBAES2007862, TBAES2007964, TBAES2008133, TBAES2009387, TCERX2000171, TCERX2000431, TCERX2000613, TCERX2000998, TCERX2002644, TCOLN1000037, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474, TESOP2001796, TESOP2001818, TESOP2001849, TESOP2001865, TESOP2001901, TESOP2002005, TESOP2002110, TESOP2002150, TESOP2002273, TESOP2002489, TESOP2002539, TESOP2002545, TESOP2002804, TESOP2002950,

TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2006893, TESOP2007041, TESOP2007052, TESOP2007262, TESOP2007636, TESOP2007674, TESOP2007688, TESOP2007725, TESOP2007912, TESOP2007978, TESOP2008127, TESOP2008552, TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000055,

TESTI1000064, TESTI1000070, TESTI1000085, TESTI1000088, TESTI1000093, TESTI1000094, TESTI1000101, TESTI1000109, TESTI1000125, TESTI1000127, TESTI1000128, TESTI1000131, TESTI1000157, TESTI1000163, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000018, TESTI2000044, TESTI2000100, TESTI2000117, TESTI2000120, TESTI2000147, TESTI2000154, TESTI2000172, TESTI2000207, TESTI2000208, TESTI2000221, TESTI2000238, TESTI2000253, TESTI2000278, TESTI2000349, TESTI2000356, TESTI2000358, TESTI2000372, TESTI2000427, TESTI2000435, TESTI2000443, TESTI2000452,

TESTI2000462, TESTI2000489, TESTI2000571, TESTI2000591, TESTI2000600, TESTI2000616, TESTI2000644, TESTI2000671, TESTI2000683, TESTI2000689, TESTI2000695, TESTI2000699, TESTI2000707, TESTI2000745, TESTI2000756, TESTI2000762, TESTI2000808, TESTI2000819, TESTI2000830, TESTI2000840, TESTI2000849, TESTI2000850, TESTI2000874, TESTI2000883, TESTI2000909, TESTI2000951, TESTI2000981, TESTI2001008, TESTI2001049, TESTI2001082, TESTI2001134, TESTI2001141, TESTI2001180, TESTI2001208, TESTI2001229, TESTI2001236, TESTI2001237, TESTI2001306, TESTI2001313, TESTI2001330, TESTI2001339, TESTI2001345, TESTI2001352, TESTI2001364, TESTI2001412, TESTI2001420, TESTI2001498, TESTI2001512, TESTI2001518, TESTI2001556,

TESTI2001593, TESTI2001613, TESTI2001621, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001829, TESTI2001852, TESTI2001869, TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002149, TESTI2002216, TESTI2002245, TESTI2002251, TESTI2002264, TESTI2002282, TESTI2002293, TESTI2002294, TESTI2002326, TESTI2002351, TESTI2002361, TESTI2002406, TESTI2002415, TESTI2002423, TESTI2002453,

TESTI2002461, TESTI2002465, TESTI2002467, TESTI2002498, TESTI2002516, TESTI2002544, TESTI2002578, TESTI2002580, TESTI2002618, TESTI2002632, TESTI2002676, TESTI2002690, TESTI2002696, TESTI2002698, TESTI2002700, TESTI2002724, TESTI2002729, TESTI2002752, TESTI2002753, TESTI2002789, TESTI2002802, TESTI2002806, TESTI2002840, TESTI2002910, TESTI2002912, TESTI2002928, TESTI2002937, TESTI2002993, TESTI2003005, TESTI2003020, TESTI2003031, TESTI2003037, TESTI2003044, TESTI2003059, TESTI2003061, TESTI2003071, TESTI2003074, TESTI2003089, TESTI2003104, TESTI2003117, TESTI2003127, TESTI2003130, TESTI2003131, TESTI2003139, TESTI2003141, TESTI2003152, TESTI2003181, TESTI2003193, TESTI2003196, TESTI2003228,

TESTI2003236, TESTI2003255, TESTI2003258, TESTI2003277, TESTI2003299, TESTI2003311, TESTI2003321, TESTI2003325, TESTI2003327, TESTI2003330, TESTI2003347, TESTI2003354, TESTI2003356, TESTI2003372, TESTI2003376, TESTI2003418, TESTI2003419, TESTI2003432, TESTI2003475, TESTI2003476, TESTI2003498, TESTI2003512, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998,

TESTI2004000, TESTI2004073, TESTI2004085, TESTI2004089, TESTI2004111, TESTI2004119, TESTI2004122, TESTI2004153, TESTI2004163, TESTI2004169, TESTI2004207, TESTI2004215, TESTI2004227, TESTI2004229, TESTI2004243, TESTI2004255, TESTI2004287, TESTI2004313, TESTI2004318, TESTI2004322, TESTI2004391, TESTI2004399, TESTI2004423, TESTI2004431, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004783, TESTI2004791, TESTI2004793, TESTI2004860, TESTI2004906, TESTI2004920, TESTI2004929, TESTI2004936,

TESTI2004941, TESTI2004971, TESTI2004982, TESTI2004993, TESTI2004994, TESTI2004999, TESTI2005017, TESTI2005025, TESTI2005040, TESTI2005060, TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005415, TESTI2005470, TESTI2005492, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005703, TESTI2005720, TESTI2005739, TESTI2005742, TESTI2005767, TESTI2005775, TESTI2005827,

TESTI2005835, TESTI2005860, TESTI2005892, TESTI2005911, TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006051, TESTI2006067, TESTI2006083, TESTI2006097, TESTI2006109, TESTI2006111, TESTI2006125, TESTI2006179, TESTI2006209, TESTI2006210, TESTI2006212, TESTI2006218, TESTI2006238, TESTI2006255, TESTI2006278, TESTI2006333, TESTI2006341, TESTI2006360, TESTI2006409, TESTI2006425, TESTI2006437, TESTI2006457, TESTI2006460, TESTI2006465, TESTI2006483, TESTI2006499, TESTI2006523, TESTI2006543, TESTI2006565,

TESTI2006572, TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006735, TESTI2006744, TESTI2006748, TESTI2006774, TESTI2006853, TESTI2006866, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007220, TESTI2007234, TESTI2007311, TESTI2007320, TESTI2007346, TESTI2007358, TESTI2007402, TESTI2007405, TESTI2007452, TESTI2007466, TESTI2007480, TESTI2007490, TESTI2007502, TESTI2007524, TESTI2007533, TESTI2007576, TESTI2007613, TESTI2007657,

TESTI2007685, TESTI2007692, TESTI2007749, TESTI2007750, TESTI2007808, TESTI2007814, TESTI2007829, TESTI2007867, TESTI2007872, TESTI2007906, TESTI2007922, TESTI2007951, TESTI2007972, TESTI2007973, TESTI2008014, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008420, TESTI2008425, TESTI2008440, TESTI2008567, TESTI2008595, TESTI2008621, TESTI2008636, TESTI2008657, TESTI2008684, TESTI2008699, TESTI2008715, TESTI2008762, TESTI2008774, TESTI2008786,

TESTI2008822, TESTI2008835, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009412, TESTI2009423, TESTI2009447, TESTI2009462, TESTI2009477, TESTI2009497, TESTI2009511, TESTI2009544, TESTI2009545, TESTI2009551, TESTI2009585, TESTI2009588, TESTI2009683, TESTI2009727, TESTI2009739, TESTI2009812, TESTI2009835, TESTI2009853, TESTI2009935, TESTI2009977, TESTI2010009, TESTI2010154, TESTI2010239, TESTI2010280, TESTI2010354, TESTI2010396, TESTI2010400, TESTI2010409, TESTI2010572,

TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011680, TESTI2011683, TESTI2011712, TESTI2011750, TESTI2011840, TESTI2011846, TESTI2011915, TESTI2011928, TESTI2011971, TESTI2012019, TESTI2012050, TESTI2012090, TESTI2012095, TESTI2012104, TESTI2012152, TESTI2012155, TESTI2012171, TESTI2012207, TESTI2012231, TESTI2012244,

TESTI2012289, TESTI2012308, TESTI2012356, TESTI2012387, TESTI2012444, TESTI2012511, TESTI2012528, TESTI2012544, TESTI2012550, TESTI2012571, TESTI2012592, TESTI2012617, TESTI2012628, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013257, TESTI2013268, TESTI2013322, TESTI2013381, TESTI2013382, TESTI2013401, TESTI2013427, TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889,

TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014577, TESTI2014578, TESTI2014716, TESTI2014780, TESTI2014800, TESTI2014843, TESTI2014979, TESTI2014988, TESTI2015042, TESTI2015105, TESTI2015180, TESTI2015246, TESTI2015249, TESTI2015327, TESTI2015331, TESTI2015335, TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015987, TESTI2016046, TESTI2016101, TESTI2016162, TESTI2016197,

TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102, TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951,

TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018941, TESTI2018957, TESTI2018974, TESTI2018975, TESTI2019042, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019787, TESTI2019794, TESTI2019854, TESTI2019859, TESTI2019860, TESTI2019872,

TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020515, TESTI2020525, TESTI2020579, TESTI2020717, TESTI2020871, TESTI2020905, TESTI2020906, TESTI2020946, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021138, TESTI2021315, TESTI2021358, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895,

TESTI2021911, TESTI2021939, TESTI2021999, TESTI2022010, TESTI2022023, TESTI2022065, TESTI2022086, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874, TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267,

TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024885, TESTI2024936, TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025454, TESTI2025486, TESTI2025546, TESTI2025582, TESTI2025609, TESTI2025656, TESTI2025791, TESTI2025846, TESTI2025872, TESTI2025879, TESTI2025911, TESTI2025920, TESTI2025924, TESTI2026014, TESTI2026024,

TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491, TESTI2026505, TESTI2026515, TESTI2026525, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027165, TESTI2027171, TESTI2027173, TESTI2027179, TESTI2027185, TESTI2027206, TESTI2027238, TESTI2027239, TESTI2027243, TESTI2027296, TESTI2027378, TESTI2027411, TESTI2027450, TESTI2027496,

TESTI2027503, TESTI2027562, TESTI2027615, TESTI2027645, TESTI2027730, TESTI2027736, TESTI2027791, TESTI2027820, TESTI2027828, TESTI2027840, TESTI2028098, TESTI2028151, TESTI2028242, TESTI2028253, TESTI2028254, TESTI2028269, TESTI2028290, TESTI2028296, TESTI2028384, TESTI2028426, TESTI2028488, TESTI2028523, TESTI2028583, TESTI2028613, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029244, TESTI2029249, TESTI2029252, TESTI2029264, TESTI2029470, TESTI2029672, TESTI2029880, TESTI2030136, TESTI2030322, TESTI2030342, TESTI2030519, TESTI2030556, TESTI2030728, TESTI2030754,

TESTI2030770, TESTI2030860, TESTI2030901, TESTI2030917, TESTI2030930, TESTI2031006, TESTI2031060, TESTI2031129, TESTI2031356, TESTI2031418, TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032681, TESTI2032761, TESTI2032768, TESTI2032774, TESTI2032828, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034357, TESTI2034401, TESTI2034407, TESTI2034506, TESTI2034520, TESTI2034718, TESTI2034730, TESTI2034749, TESTI2034767, TESTI2034774, TESTI2034777,

TESTI2034785, TESTI2034799, TESTI2034847, TESTI2034913, TESTI2034931, TESTI2034953, TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036922, TESTI2036951, TESTI2036965, TESTI2037002, TESTI2037055, TESTI2037081, TESTI2037085, TESTI2037103, TESTI2037106, TESTI2037111, TESTI2037209, TESTI2037255, TESTI2037359, TESTI2037375,

TESTI2037382, TESTI2037534, TESTI2037569, TESTI2037572, TESTI2037643, TESTI2037657, TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039209, TESTI2039225, TESTI2039227, TESTI2039276, TESTI2039342, TESTI2039353, TESTI2039544, TESTI2039613, TESTI2039732, TESTI2039738, TESTI2039776, TESTI2039867, TESTI2039908, TESTI2040018, TESTI2040094, TESTI2040102, TESTI2040128, TESTI2040143,

TESTI2040297, TESTI2040372, TESTI2040377, TESTI2040424, TESTI2040642, TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866,

TESTI2043869, TESTI2044064, TESTI2044118, TESTI2044194, TESTI2044306, TESTI2044309, TESTI2044413, TESTI2044418, TESTI2044754, TESTI2044788, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212,

TESTI2047342, TESTI2047383, TESTI2047792, TESTI2047801, TESTI2047818, TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698,

TESTI2052799, TESTI2052822, TESTI2052985, TESTI2053151, TESTI2053242, TESTI2053399, TESTI2053526, TESTI2053561, TESTI2053621, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000370, TESTI4000394, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001473,

TESTI4001517, TESTI4001527, TESTI4001569, TESTI4001604, TESTI4001665, TESTI4001679, TESTI4001795, TESTI4001923, TESTI4001925, TESTI4001984, TESTI4002003, TESTI4002072, TESTI4002141, TESTI4002195, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4003279, TESTI4003319, TESTI4003404, TESTI4003565, TESTI4003574, TESTI4003733, TESTI4003796, TESTI4003944, TESTI4004003, TESTI4004031, TESTI4004200, TESTI4004539, TESTI4004626, TESTI4004653, TESTI4004695, TESTI4004722, TESTI4004887, TESTI4004917, TESTI4005015, TESTI4005039, TESTI4005158, TESTI4005317, TESTI4005322, TESTI4005470, TESTI4005500,

TESTI4005534, TESTI4005543, TESTI4005628, TESTI4005801, TESTI4005805, TESTI4005857, TESTI4005961, TESTI4006053, TESTI4006079, TESTI4006112, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007965, TESTI4008007, TESTI4008018, TESTI4008050, TESTI4008058, TESTI4008086, TESTI4008097,

TESTI4008176, TESTI4008209, TESTI4008219, TESTI4008250, TESTI4008302, TESTI4008305, TESTI4008401, TESTI4008417, TESTI4008429, TESTI4008573, TESTI4008797, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789, TESTI4010817, TESTI4010831, TESTI4010902, TESTI4010928, TESTI4011070, TESTI4011072, TESTI4011084,

TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013602, TESTI4013667, TESTI4013675, TESTI4013685, TESTI4013742, TESTI4013774, TESTI4013817, TESTI4013830, TESTI4013852, TESTI4013894, TESTI4013924, TESTI4013960, TESTI4013962, TESTI4014159, TESTI4014175, TESTI4014265, TESTI4014276, TESTI4014306, TESTI4014392,

TESTI4014415, TESTI4014445, TESTI4014553, TESTI4014661, TESTI4014694, TESTI4014801, TESTI4014818, TESTI4014891, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4017001, TESTI4017116, TESTI4017137, TESTI4017229, TESTI4017254, TESTI4017269, TESTI4017380, TESTI4017382, TESTI4017543, TESTI4017575, TESTI4017647, TESTI4017714, TESTI4017763, TESTI4017848, TESTI4017854,

TESTI4017901, TESTI4017961, TESTI4017984, TESTI4018101, TESTI4018208, TESTI4018382, TESTI4018436, TESTI4018506, TESTI4018555, TESTI4018751, TESTI4018806, TESTI4018835, TESTI4018859, TESTI4018881, TESTI4018886, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569, TESTI4021713, TESTI4021821, TESTI4022158, TESTI4022577, TESTI4022716, TESTI4022873, TESTI4023096, TESTI4023172, TESTI4023546, TESTI4023555, TESTI4023654,

TESTI4023722, TESTI4023762, TESTI4023942, TESTI4024096, TESTI4024240, TESTI4024294, TESTI4024344, TESTI4024387, TESTI4024420, TESTI4024494, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139, TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612,

TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856, TESTI4032895, TESTI4033146, TESTI4033177, TESTI4033433, TESTI4034172, TESTI4034212, TESTI4034432, TESTI4034495, TESTI4034632, TESTI4034633, TESTI4034912, TESTI4034973, TESTI4035063,

TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038339, TESTI4038492, TESTI4038721, TESTI4038758, TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800,

TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043166, TESTI4043203, TESTI4043223, TESTI4043371, TESTI4043378, TESTI4043512, TESTI4043551, TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044770, TESTI4045168, TESTI4045312, TESTI4045330,

TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, TUTER1000014, TUTER1000137, TUTER2000057, TUTER2000283, TUTER2000425, TUTER2000916, TUTER2001286, TUTER2001341, TUTER2001387, TUTER2001433, TUTER2001461, TUTER2002028, TUTER2002074, TUTER2002158, TUTER2002228, TUTER2002356, TUTER2002729, UMVEN1000122, UMVEN1000156, UMVEN1000186,

UMVEN2000046, UMVEN2000069, UTERU1000015, UTERU1000024, UTERU1000031, UTERU1000032, UTERU1000057, UTERU1000065, UTERU1000077, UTERU1000093, UTERU1000096, UTERU1000109, UTERU1000131, UTERU1000138, UTERU1000148, UTERU1000160, UTERU1000183, UTERU1000187, UTERU1000192, UTERU1000249, UTERU1000337, UTERU1000339, UTERU2000023, UTERU2000047, UTERU2000074, UTERU2000099, UTERU2000154, UTERU2000197, UTERU2000218, UTERU2000238, UTERU2000243, UTERU2000300, UTERU2000329, UTERU2000332, UTERU2000338, UTERU2000349, UTERU2000377, UTERU2000393, UTERU2000418, UTERU2000465, UTERU2000485, UTERU2000517, UTERU2000524, UTERU2000539, UTERU2000541, UTERU2000542, UTERU2000546, UTERU2000550, UTERU2000569, UTERU2000607,

UTERU2000629, UTERU2000649, UTERU2000663, UTERU2000696, UTERU2000794, UTERU2000830, UTERU2000844, UTERU2000922, UTERU2000925, UTERU2001024, UTERU2001110, UTERU2001176, UTERU2001281, UTERU2001389, UTERU2001409, UTERU2001412, UTERU2001504, UTERU2001658, UTERU2001747, UTERU2001876, UTERU2002001, UTERU2002011, UTERU2002176, UTERU2002198, UTERU2002332, UTERU2002473, UTERU2002547, UTERU2002693, UTERU2002733, UTERU2002736, UTERU2002737, UTERU2002826, UTERU2002841, UTERU2002964, UTERU2002993, UTERU2003035, UTERU2003057, UTERU2003126, UTERU2003135, UTERU2003321, UTERU2003399, UTERU2003411, UTERU2003456, UTERU2003577, UTERU2003926, UTERU2003973, UTERU2004015, UTERU2004039, UTERU2004061, UTERU2004073,

UTERU2004163, UTERU2004299, UTERU2004461, UTERU2004520, UTERU2004564, UTERU2004664, UTERU2004688, UTERU2004698, UTERU2004929, UTERU2005050, UTERU2005069, UTERU2005179, UTERU2005346, UTERU2005446, UTERU2005450, UTERU2005533, UTERU2005664, UTERU2005822, UTERU2005903, UTERU2005905, UTERU2006103, UTERU2006137, UTERU2006182, UTERU2006400, UTERU2006412, UTERU2006429, UTERU2006486, UTERU2006524, UTERU2006547, UTERU2006568, UTERU2006643, UTERU2006651, UTERU2006705, UTERU2006899, UTERU2007004, UTERU2007075, UTERU2007081, UTERU2007128, UTERU2007253, UTERU2007267, UTERU2007444, UTERU2007499, UTERU2007639, UTERU2007724, UTERU2007924, UTERU2007942, UTERU2008018, UTERU2008019, UTERU2008027, UTERU2008040,

UTERU2008077, UTERU2008130, UTERU2008347, UTERU2008516, UTERU2008561, UTERU2008653, UTERU2008705, UTERU2008707, UTERU2008747, UTERU2008785, UTERU2008845, UTERU2008901, UTERU2008930, UTERU2008938, UTERU2008939, UTERU2008962, UTERU2009094, UTERU2009120, UTERU2009131, UTERU2009147, UTERU2009206, UTERU2009283, UTERU2009414, UTERU2009483, UTERU2009510, UTERU2009538, UTERU2009540, UTERU2009776, UTERU2009904, UTERU2009951, UTERU2010115, UTERU2010124, UTERU2010164, UTERU2010231, UTERU2010304, UTERU2010320, UTERU2010431, UTERU2010525, UTERU2010651, UTERU2010724, UTERU2010747, UTERU2011195, UTERU2011199, UTERU2011261, UTERU2011410, UTERU2011574, UTERU2011657, UTERU2011806, UTERU2011811, UTERU2011897,

UTERU2011962, UTERU2011968, UTERU2012031, UTERU2012101, UTERU2012114, UTERU2012252, UTERU2012333, UTERU2012407, UTERU2012526, UTERU2012581, UTERU2012610, UTERU2012615, UTERU2012703, UTERU2012715, UTERU2012786, UTERU2012856, UTERU2012890, UTERU2012938, UTERU2012976, UTERU2013048, UTERU2013078, UTERU2013231, UTERU2013262, UTERU2013280, UTERU2013322, UTERU2013483, UTERU2013491, UTERU2013502, UTERU2013586, UTERU2013926, UTERU2013976, UTERU2014001, UTERU2014024, UTERU2014167, UTERU2014223, UTERU2014398, UTERU2014464, UTERU2014548, UTERU2014601, UTERU2014631, UTERU2014668, UTERU2014678, UTERU2014728, UTERU2014898, UTERU2014998, UTERU2015062, UTERU2015087, UTERU2015108, UTERU2015190, UTERU2015198,

UTERU2015202, UTERU2015405, UTERU2015640, UTERU2015880, UTERU2016147, UTERU2016157, UTERU2016426, UTERU2016464, UTERU2016669, UTERU2016757, UTERU2016761, UTERU2016896, UTERU2016902, UTERU2016979, UTERU2016981, UTERU2017123, UTERU2017303, UTERU2017421, UTERU2017492, UTERU2017613, UTERU2017623, UTERU2017632, UTERU2017761, UTERU2017762, UTERU2017810, UTERU2017988, UTERU2018127, UTERU2018180, UTERU2018200, UTERU2018333, UTERU2018364, UTERU2018514, UTERU2018522, UTERU2018523, UTERU2018544, UTERU2018566, UTERU2018609, UTERU2018674, UTERU2018712, UTERU2018784, UTERU2018789, UTERU2018811, UTERU2018867, UTERU2018881, UTERU2018955, UTERU2019038, UTERU2019163, UTERU2019257, UTERU2019453, UTERU2019491,

UTERU2019534, UTERU2019681, UTERU2019706, UTERU2019710, UTERU2019940, UTERU2019959, UTERU2019964, UTERU2020226, UTERU2020242, UTERU2020292, UTERU2020351, UTERU2020491, UTERU2020718, UTERU2021163, UTERU2021380, UTERU2021649, UTERU2021820, UTERU2022020, UTERU2022773, UTERU2022955, UTERU2022981, UTERU2023039, UTERU2023045, UTERU2023262, UTERU2023687, UTERU2023712, UTERU2023941, UTERU2024002, UTERU2024042, UTERU2024141, UTERU2024481, UTERU2024758, UTERU2024820, UTERU2024881, UTERU2025025, UTERU2025041, UTERU2025301, UTERU2025366, UTERU2025415, UTERU2025579, UTERU2025645, UTERU2025891, UTERU2026025, UTERU2026090, UTERU2026142, UTERU2026203, UTERU2026775, UTERU2027023, UTERU2027369, UTERU2027591,

UTERU2027616, UTERU2027941, UTERU2028377, UTERU2029503, UTERU2029660, UTERU2029742, UTERU2029953, UTERU2030213, UTERU2030270, UTERU2030280, UTERU2031060, UTERU2031268, UTERU2031295, UTERU2031521, UTERU2031611, UTERU2031703, UTERU2031834, UTERU2031851, UTERU2032075, UTERU2032220, UTERU2032279, UTERU2032726, UTERU2033172, UTERU2033375, UTERU2033382, UTERU2033420, UTERU2033530, UTERU2033577, UTERU2034053, UTERU2034695, UTERU2035187, UTERU2035231, UTERU2035306, UTERU2035323, UTERU2035328, UTERU2035331, UTERU2035452, UTERU2035469, UTERU2035503, UTERU2035745, UTERU2035908, UTERU2035926, UTERU2035978, UTERU2036089, UTERU2036507, UTERU2036512, UTERU2036530, UTERU2036623, UTERU2036690, UTERU2037224,

UTERU2037361, UTERU2037423, UTERU2037577, UTERU2037674, UTERU2037791, UTERU2037843, UTERU2038171, UTERU2038251, UTERU3000226, UTERU3000402, UTERU3000665, UTERU3000670, UTERU3000727, UTERU3000828, UTERU3000844, UTERU3000899, UTERU3000959, UTERU3001029, UTERU3001053, UTERU3001059, UTERU3001158, UTERU3001240, UTERU3001394, UTERU3001558, UTERU3001571, UTERU3001585, UTERU3001632, UTERU3001652, UTERU3001766, UTERU3001988, UTERU3002209, UTERU3002383, UTERU3002600, UTERU3002620, UTERU3002667, UTERU3002701, UTERU3002731, UTERU3002768, UTERU3002993, UTERU3003116, UTERU3003178, UTERU3003465, UTERU3003495, UTERU3003523, UTERU3003774, UTERU3003776, UTERU3004384, UTERU3004418, UTERU3004477, UTERU3004523,

UTERU3004616, UTERU3004635, UTERU3004709, UTERU3004938, UTERU3004992, UTERU3005159, UTERU3005205, UTERU3005230, UTERU3005264, UTERU3005460, UTERU3005536, UTERU3005585, UTERU3005767, UTERU3005907, UTERU3005970, UTERU3006008, UTERU3006228, UTERU3006308, UTERU3006538, UTERU3006640, UTERU3006687, UTERU3006720, UTERU3006798, UTERU3006884, UTERU3007108, UTERU3007134, UTERU3007419, UTERU3007640, UTERU3007731, UTERU3007913, UTERU3008280, UTERU3008463, UTERU3008660, UTERU3008671, UTERU3008722, UTERU3008917, UTERU3009259, UTERU3009490, UTERU3009517, UTERU3009690, UTERU3009775, UTERU3009871, UTERU3009979, UTERU3009987, UTERU3010029, UTERU3010526, UTERU3010604, UTERU3010892, UTERU3010919, UTERU3011063,

UTERU3011092, UTERU3011273, UTERU3011398, UTERU3011558, UTERU3011795, UTERU3011837, UTERU3012293, UTERU3012414, UTERU3012476, UTERU3012599, UTERU3012999, UTERU3013167, UTERU3013781, UTERU3014274, UTERU3014446, UTERU3014611, UTERU3014647, UTERU3014791, UTERU3014906, UTERU3015011, UTERU3015086, UTERU3015299, UTERU3015412, UTERU3015500, UTERU3015647, UTERU3015844, UTERU3016070, UTERU3016273, UTERU3016274, UTERU3016308, UTERU3016789, UTERU3017176, UTERU3017441, UTERU3017626, UTERU3017995, UTERU3018154, UTERU3018255, UTERU3018616, UTERU3019078

Of the 17,176 genes that include the cDNAs of the present invention, genes whose expression levels were high enough to detect in each of the TUTER, UMVEN, and UTERU libraries were analyzed for expression frequency. The analysis results are described below. Each is a relative value, where the expression level over all tissues is taken as 100. The larger this value, the greater the level of expression.

TABLE 40

| Clone Name | TUTER | UMVEN | UTERU |
|---|---|---|---|
| 3NB691000173 | 0 | 0.000 | 0.942 |
| 3NB692000281 | 9.812 | 0 | 0.525 |
| 3NB692000545 | 0 | 0.000 | 3.365 |
| 3NB692000912 | 0 | 0.000 | 0.560 |
| 3NB692001334 | 5.982 | 0 | 0.000 |
| 3NB692001853 | 0 | 0.000 | 3.123 |
| ADRGL2000006 | 0 | 0.000 | 1.240 |
| ADRGL2000042 | 0 | 0.000 | 3.029 |
| ADRGL2000328 | 0 | 0.000 | 8.029 |
| ADRGL2002013 | 0 | 0.000 | 10.729 |
| ADRGL2004676 | 0 | 0.000 | 5.199 |
| ADRGL2009273 | 0 | 0.000 | 1.657 |
| ASTRO2000046 | 0 | 0.000 | 0.487 |
| ASTRO2000480 | 0 | 0.000 | 3.377 |
| ASTRO2000801 | 0 | 0.000 | 1.217 |
| ASTRO2000914 | 43.380 | 0 | 0.000 |
| ASTRO2001076 | 0 | 0.000 | 1.695 |
| ASTRO2001823 | 0 | 0.000 | 1.731 |
| ASTRO2003024 | 21.123 | 0 | 2.261 |
| ASTRO2003316 | 0 | 0.000 | 10.555 |
| ASTRO2003925 | 0 | 0.000 | 25.609 |
| ASTRO2005863 | 0 | 0.000 | 4.534 |
| ASTRO2006732 | 0 | 0.000 | 18.293 |
| ASTRO2007047 | 0 | 0.000 | 4.985 |
| ASTRO2007948 | 0 | 0.000 | 2.987 |
| ASTRO2008216 | 0 | 0.000 | 3.806 |
| ASTRO2008508 | 0 | 0.000 | 10.726 |
| ASTRO2010582 | 0 | 0.000 | 20.644 |
| ASTRO2010819 | 0 | 0.000 | 3.210 |
| ASTRO2013585 | 0 | 0.000 | 0.581 |
| ASTRO2016491 | 0 | 0.000 | 0.601 |
| BGGI12000054 | 0 | 0.000 | 0.281 |
| BGGI12001002 | 0 | 0.000 | 4.235 |
| BLADE2004722 | 0 | 0.000 | 11.937 |
| BLADE2006412 | 0 | 0.000 | 7.241 |
| BLADE2007589 | 0 | 0.000 | 11.792 |
| BNGH41000137 | 0 | 0.000 | 15.524 |
| BNGH42000739 | 0 | 0.000 | 0.742 |
| BNGH42002487 | 0 | 0.000 | 11.163 |
| BNGH42006135 | 0 | 0.000 | 7.087 |
| BNGH42007460 | 0 | 0.000 | 2.404 |
| BNGH42007709 | 0 | 0.000 | 1.373 |
| BNGH42007788 | 0 | 0.000 | 3.672 |
| BRACE1000042 | 0 | 0.000 | 7.262 |
| BRACE1000070 | 0 | 0.000 | 2.132 |
| BRACE1000131 | 0 | 0.000 | 3.727 |
| BRACE1000186 | 0 | 0.000 | 2.579 |
| BRACE2000347 | 0 | 0.000 | 6.166 |
| BRACE2000750 | 0 | 0.000 | 24.547 |
| BRACE2001065 | 0 | 0.000 | 7.920 |
| BRACE2001143 | 0 | 0.000 | 1.772 |
| BRACE2001188 | 0 | 0.000 | 3.367 |
| BRACE2001692 | 0 | 0.000 | 0.722 |
| BRACE2001855 | 0 | 0.000 | 1.372 |
| BRACE2001971 | 0 | 0.000 | 2.675 |
| BRACE2002176 | 0 | 0.000 | 62.692 |
| BRACE2002409 | 0 | 0.000 | 62.692 |
| BRACE2002478 | 0 | 0.000 | 17.287 |
| BRACE2002649 | 0 | 0.000 | 12.628 |
| BRACE2002677 | 0 | 0.000 | 6.908 |
| BRACE2003172 | 0 | 0.000 | 6.590 |
| BRACE2003254 | 0 | 0.000 | 1.080 |
| BRACE2003512 | 0 | 0.000 | 77.068 |
| BRACE2003516 | 0 | 0.000 | 62.692 |
| BRACE2003527 | 0 | 0.000 | 3.074 |
| BRACE2003785 | 0 | 0.000 | 1.159 |
| BRACE2003846 | 0 | 0.000 | 0.777 |
| BRACE2003892 | 0 | 0.000 | 2.603 |
| BRACE2003904 | 0 | 0.000 | 3.092 |
| BRACE2005087 | 0 | 0.000 | 8.660 |
| BRACE2005196 | 0 | 0.000 | 37.926 |
| BRACE2005231 | 0 | 0.000 | 3.808 |
| BRACE2005290 | 0 | 0.000 | 30.346 |
| BRACE2005719 | 0 | 0.000 | 3.542 |
| BRACE2006084 | 0 | 0.000 | 3.661 |
| BRACE2006354 | 0 | 0.000 | 7.422 |
| BRACE2006397 | 6.578 | 0 | 0.352 |
| BRACE2006537 | 0 | 0.000 | 2.567 |
| BRACE2006636 | 0 | 0.000 | 16.351 |
| BRACE2006900 | 0 | 0.000 | 1.227 |
| BRACE2006944 | 0 | 0.000 | 13.911 |
| BRACE2007064 | 0 | 0.000 | 3.876 |
| BRACE2007174 | 0 | 0.000 | 18.633 |
| BRACE2007518 | 0 | 0.000 | 2.707 |
| BRACE2007798 | 0 | 0.000 | 1.234 |
| BRACE2008653 | 0 | 0.000 | 4.403 |
| BRACE2008941 | 0 | 0.000 | 11.920 |
| BRACE2009037 | 0 | 0.000 | 0.697 |
| BRACE2009517 | 0 | 0.000 | 6.470 |
| BRACE2011183 | 0 | 0.000 | 7.667 |
| BRACE2012528 | 0 | 0.000 | 0.566 |
| BRACE2017397 | 0 | 0.000 | 11.252 |
| BRACE2017574 | 0 | 0.000 | 6.110 |
| BRACE2019147 | 0 | 0.000 | 2.554 |
| BRACE2020584 | 0 | 0.000 | 62.692 |
| BRACE2022333 | 0 | 0.000 | 10.589 |
| BRACE2022638 | 0 | 0.000 | 2.667 |
| BRACE2027896 | 0 | 0.000 | 30.503 |
| BRACE2030341 | 0 | 0.000 | 16.499 |
| BRACE2037310 | 0 | 0.000 | 23.401 |
| BRACE2045445 | 0 | 0.000 | 62.692 |
| BRACE3004371 | 0 | 0.000 | 4.481 |
| BRACE3005938 | 0 | 0.000 | 1.313 |
| BRACE3007258 | 0 | 0.000 | 15.521 |
| BRACE3008772 | 0 | 0.000 | 62.692 |
| BRACE3010428 | 0 | 0.000 | 5.461 |
| BRACE3027478 | 0 | 0.000 | 19.089 |
| BRACE3040863 | 0 | 0.000 | 21.335 |
| BRALZ2009446 | 0 | 0.000 | 5.561 |
| BRALZ2009482 | 0 | 0.000 | 0.443 |
| BRALZ2017359 | 0 | 0.000 | 24.816 |
| BRAMY2000585 | 0 | 0.000 | 2.929 |
| BRAMY2004363 | 0 | 0.000 | 12.191 |
| BRAMY2004521 | 0 | 0.000 | 5.654 |
| BRAMY2006366 | 0 | 0.000 | 10.987 |
| BRAMY2009123 | 0 | 0.000 | 54.206 |
| BRAMY2009934 | 0 | 0.000 | 7.051 |
| BRAMY2011105 | 67.510 | 0 | 0.000 |
| BRAMY2012536 | 0 | 0.000 | 0.688 |
| BRAMY2013659 | 0 | 0.000 | 34.645 |
| BRAMY2019989 | 0 | 0.000 | 1.150 |
| BRAMY2020427 | 0 | 0.000 | 0.706 |
| BRAMY2022168 | 0 | 0.000 | 16.134 |
| BRAMY2026685 | 0 | 0.000 | 2.956 |
| BRAMY2028740 | 0 | 0.000 | 1.110 |
| BRAMY2031516 | 0 | 0.000 | 8.809 |
| BRAMY2032087 | 0 | 0.000 | 4.333 |
| BRAMY2040095 | 0 | 0.000 | 0.815 |
| BRAMY3002458 | 0 | 0.000 | 14.807 |
| BRAMY3005184 | 0 | 0.000 | 26.725 |
| BRAWH1000040 | 0 | 0.000 | 2.462 |
| BRAWH1000093 | 10.279 | 0 | 2.200 |
| BRAWH2000082 | 0 | 0.000 | 0.457 |
| BRAWH2000488 | 0 | 0.000 | 12.025 |
| BRAWH2000588 | 0 | 0.000 | 1.496 |
| BRAWH2000633 | 0 | 0.000 | 1.581 |
| BRAWH2000697 | 0 | 0.000 | 1.206 |
| BRAWH2000839 | 0 | 0.000 | 0.980 |
| BRAWH2001092 | 0 | 0.000 | 1.677 |
| BRAWH2001141 | 0 | 0.000 | 6.061 |
| BRAWH2001412 | 0 | 0.000 | 17.933 |
| BRAWH2001439 | 0 | 0.000 | 1.227 |
| BRAWH2002601 | 0 | 0.000 | 5.932 |
| BRAWH2003693 | 0 | 0.000 | 4.080 |
| BRAWH2004078 | 0 | 0.000 | 6.285 |
| BRAWH2005578 | 0 | 0.000 | 15.983 |
| BRAWH2005661 | 0 | 0.000 | 1.907 |
| BRAWH2007605 | 0 | 0.000 | 2.446 |
| BRAWH2008058 | 0 | 0.000 | 27.939 |
| BRAWH2009238 | 0 | 0.000 | 6.217 |
| BRAWH2011343 | 0 | 0.000 | 8.367 |
| BRAWH2012698 | 0 | 0.000 | 22.868 |
| BRAWH2014188 | 0 | 0.000 | 11.100 |
| BRAWH2014473 | 0 | 0.000 | 54.394 |
| BRAWH2014645 | 0 | 0.000 | 2.522 |
| BRAWH2016166 | 0 | 0.000 | 18.040 |
| BRAWH2017304 | 0 | 0.000 | 19.804 |
| BRAWH2017685 | 0 | 0.000 | 1.211 |
| BRAWH2018526 | 0 | 0.000 | 0.367 |
| BRAWH2018745 | 0 | 0.000 | 54.394 |
| BRAWH3000314 | 0 | 0.000 | 28.447 |
| BRAWH3001326 | 0 | 0.000 | 54.394 |
| BRAWH3002574 | 0 | 0.000 | 7.885 |
| BRAWH3002853 | 0 | 0.000 | 1.764 |
| BRAWH3003727 | 0 | 0.000 | 11.993 |
| BRAWH3004350 | 0 | 0.000 | 19.774 |
| BRAWH3016271 | 0 | 0.000 | 6.433 |
| BRAWH3021545 | 0 | 0.000 | 17.404 |
| BRAWH3026529 | 0 | 0.000 | 54.394 |
| BRAWH3029806 | 0 | 0.000 | 10.629 |
| BRCAN2003944 | 0 | 0.000 | 0.336 |
| BRCAN2009432 | 29.345 | 0 | 3.141 |
| BRCAN2011946 | 0 | 0.000 | 7.542 |
| BRCAN2021024 | 0 | 0.000 | 24.461 |
| BRCAN2022126 | 0 | 0.000 | 5.791 |
| BRCAN2024572 | 0 | 0.000 | 3.257 |
| BRCAN2028355 | 0 | 0.000 | 1.762 |
| BRCOC1000040 | 0 | 0.000 | 14.069 |
| BRCOC2000333 | 0 | 0.000 | 14.286 |
| BRCOC2003187 | 0 | 0.000 | 0.938 |
| BRCOC2010730 | 0 | 0.000 | 12.892 |
| BRCOC2012172 | 0 | 0.000 | 6.710 |
| BRCOC2012551 | 0 | 0.000 | 8.602 |
| BRCOC2015597 | 0 | 0.000 | 25.187 |
| BRHIP2001099 | 0 | 0.000 | 8.268 |
| BRHIP2003062 | 0 | 0.000 | 56.647 |
| BRHIP2007305 | 0 | 0.000 | 1.350 |
| BRHIP2010487 | 91.660 | 0 | 0.000 |
| BRHIP2010571 | 0 | 0.000 | 18.366 |
| BRHIP2011491 | 0 | 0.000 | 0.986 |
| BRHIP2011616 | 0 | 0.000 | 53.896 |
| BRHIP2015245 | 0 | 0.000 | 5.663 |
| BRHIP2018712 | 0 | 0.000 | 9.661 |
| BRHIP2019149 | 0 | 0.000 | 13.172 |
| BRHIP2020799 | 0 | 0.000 | 12.072 |
| BRHIP2021762 | 0 | 0.000 | 6.646 |
| BRHIP2022228 | 0 | 0.000 | 10.898 |
| BRHIP2022326 | 0 | 0.000 | 1.015 |
| BRHIP2023888 | 0 | 0.000 | 0.439 |
| BRHIP2024742 | 0 | 0.000 | 1.487 |
| BRHIP2024911 | 0 | 0.000 | 11.054 |
| BRHIP2027017 | 0 | 0.000 | 13.730 |
| BRHIP3000017 | 0 | 0.000 | 15.777 |
| BRHIP3000377 | 0 | 0.000 | 6.870 |
| BRHIP3001076 | 0 | 0.000 | 53.896 |
| BRHIP3002141 | 0 | 0.000 | 18.713 |
| BRHIP3005307 | 0 | 0.000 | 53.896 |
| BRHIP3007223 | 0 | 0.000 | 2.405 |
| BRHIP3007586 | 0 | 0.000 | 4.116 |
| BRHIP3008344 | 0 | 0.000 | 53.896 |
| BRHIP3008565 | 0 | 0.000 | 53.896 |
| BRHIP3012997 | 0 | 87.379 | 0.000 |
| BRHIP3017855 | 0 | 0.000 | 27.401 |
| BRHIP3020046 | 0 | 0.000 | 1.208 |
| BRSSN2000295 | 0 | 0.000 | 8.260 |
| BRSSN2000498 | 0 | 0.000 | 2.610 |
| BRSSN2001213 | 0 | 0.000 | 24.268 |
| BRSSN2001869 | 0 | 0.000 | 3.600 |
| BRSSN2006892 | 0 | 0.000 | 18.468 |
| BRSSN2009518 | 0 | 0.000 | 2.054 |
| BRSSN2010019 | 0 | 0.000 | 13.842 |
| BRSSN2014685 | 0 | 0.000 | 16.936 |
| BRSSN2017422 | 0 | 0.000 | 17.148 |
| BRSTN2001067 | 0 | 0.000 | 7.384 |
| BRSTN2010089 | 0 | 0.000 | 7.869 |
| BRSTN2011961 | 0 | 0.000 | 0.345 |
| BRSTN2012069 | 3.583 | 1.784 | 0.271 |
| BRSTN2012174 | 0 | 0.000 | 0.482 |
| BRSTN2013502 | 0 | 0.000 | 5.797 |
| BRSTN2013931 | 0 | 0.000 | 3.457 |
| BRSTN2016470 | 0 | 0.000 | 0.522 |
| BRSTN2016892 | 0 | 0.000 | 3.559 |
| BRTHA2000057 | 0 | 0.000 | 51.819 |
| BRTHA2004361 | 0 | 0.000 | 51.819 |
| BRTHA2005831 | 0 | 0.000 | 51.819 |
| BRTHA2005864 | 0 | 0.000 | 1.935 |
| BRTHA2010608 | 0 | 0.000 | 51.819 |
| BRTHA2012183 | 0 | 0.000 | 51.819 |
| BRTHA2012714 | 0 | 0.000 | 5.980 |
| BRTHA2017178 | 0 | 0.000 | 51.819 |
| BRTHA3003023 | 0 | 0.000 | 2.410 |
| BRTHA3003074 | 0 | 0.000 | 13.429 |
| BRTHA3003736 | 0 | 0.000 | 5.131 |
| BRTHA3004307 | 0 | 0.000 | 17.964 |
| BRTHA3011265 | 0 | 0.000 | 10.176 |
| BRTHA3020314 | 0 | 0.000 | 12.144 |
| BRTHA3023403 | 0 | 0.000 | 26.579 |
| BRTHA3026507 | 0 | 0.000 | 4.167 |
| BRTHA3027879 | 0 | 0.000 | 51.819 |
| CHONS2002829 | 0 | 0.000 | 11.691 |
| CTONG1000052 | 0 | 0.000 | 2.162 |
| CTONG1000087 | 0 | 0.000 | 2.494 |
| CTONG1000094 | 0 | 0.000 | 0.464 |
| CTONG1000137 | 0 | 0.000 | 39.037 |
| CTONG1000180 | 0 | 0.000 | 28.641 |
| CTONG1000241 | 0 | 0.000 | 7.590 |
| CTONG1000277 | 0 | 0.000 | 24.668 |
| CTONG1000467 | 0 | 0.000 | 10.248 |
| CTONG2000411 | 0 | 0.000 | 6.864 |
| CTONG2000508 | 0 | 0.000 | 4.709 |
| CTONG2001748 | 0 | 0.000 | 2.493 |
| CTONG2001932 | 0 | 0.000 | 12.322 |
| CTONG2002073 | 32.643 | 0 | 1.747 |
| CTONG2002270 | 0 | 0.000 | 0.633 |
| CTONG2002744 | 0 | 0.000 | 0.702 |
| CTONG2002766 | 0 | 0.000 | 14.092 |
| CTONG2002803 | 32.814 | 0 | 0.000 |
| CTONG2003298 | 0 | 0.000 | 1.731 |
| CTONG2003348 | 0 | 0.000 | 39.037 |
| CTONG2003517 | 0 | 0.000 | 13.371 |
| CTONG2003524 | 0 | 0.000 | 1.922 |
| CTONG2004423 | 0 | 0.000 | 13.705 |
| CTONG2005145 | 0 | 0.000 | 29.265 |
| CTONG2005615 | 0 | 0.000 | 5.671 |
| CTONG2006235 | 0 | 0.000 | 1.954 |
| CTONG2006377 | 0 | 0.000 | 14.002 |
| CTONG2006562 | 0 | 0.000 | 5.010 |
| CTONG2006932 | 0 | 0.000 | 2.625 |
| CTONG2007078 | 0 | 0.000 | 3.048 |
| CTONG2007500 | 0 | 0.000 | 8.072 |
| CTONG2008343 | 0 | 0.000 | 39.037 |
| CTONG2008398 | 0 | 0.000 | 0.912 |
| CTONG2008518 | 0 | 0.000 | 4.593 |
| CTONG2008689 | 0 | 0.000 | 24.924 |
| CTONG2009529 | 0 | 0.000 | 7.541 |
| CTONG2009923 | 0 | 0.000 | 39.037 |
| CTONG2009938 | 0 | 0.000 | 23.863 |
| CTONG2010348 | 0 | 0.000 | 6.336 |
| CTONG2010408 | 0 | 0.000 | 10.219 |
| CTONG2010649 | 0 | 0.000 | 5.595 |
| CTONG2010821 | 0 | 0.000 | 2.557 |
| CTONG2011801 | 0 | 0.000 | 3.532 |
| CTONG2011825 | 0 | 0.000 | 10.239 |
| CTONG2012452 | 7.936 | 0 | 0.000 |
| CTONG2012847 | 38.378 | 0 | 2.054 |
| CTONG2012879 | 0 | 0.000 | 4.056 |
| CTONG2012996 | 0 | 0.000 | 3.482 |
| CTONG2013128 | 0 | 0.000 | 39.037 |
| CTONG2013156 | 0 | 0.000 | 24.668 |
| CTONG2013907 | 0 | 0.000 | 2.571 |
| CTONG2013934 | 0 | 0.000 | 8.273 |
| CTONG2014369 | 0 | 0.000 | 4.963 |
| CTONG2014898 | 0 | 0.000 | 13.759 |
| CTONG2015345 | 0 | 0.000 | 10.182 |
| CTONG2016056 | 0 | 0.000 | 2.372 |
| CTONG2016408 | 0 | 0.000 | 4.416 |
| CTONG2016575 | 0 | 0.000 | 1.779 |
| CTONG2016869 | 0 | 0.000 | 3.150 |
| CTONG2017094 | 0 | 0.000 | 2.666 |
| CTONG2017429 | 0 | 0.000 | 8.607 |
| CTONG2017998 | 0 | 0.000 | 5.976 |
| CTONG2018211 | 0 | 0.000 | 4.068 |
| CTONG2018637 | 0 | 0.000 | 15.649 |
| CTONG2018898 | 0 | 0.000 | 2.298 |
| CTONG2019822 | 0 | 0.000 | 7.755 |
| CTONG2020378 | 0 | 0.000 | 19.250 |
| CTONG2020411 | 0 | 0.000 | 32.844 |
| CTONG2020484 | 0 | 55.423 | 0.000 |
| CTONG2020560 | 0 | 0.000 | 5.368 |
| CTONG2024031 | 0 | 0.000 | 6.165 |
| CTONG2027263 | 0 | 0.000 | 19.641 |
| CTONG2028124 | 0 | 0.000 | 2.997 |
| CTONG3001123 | 0 | 0.000 | 9.359 |
| CTONG3002518 | 0 | 0.000 | 10.064 |
| CTONG3004317 | 0 | 0.000 | 39.037 |
| CTONG3008894 | 0 | 0.000 | 0.867 |
| CTONG3009028 | 0 | 0.000 | 3.839 |
| CTONG3009239 | 0 | 0.000 | 1.722 |
| D3OST3000258 | 0 | 0.000 | 22.242 |
| D6OST2000464 | 0 | 0.000 | 1.307 |
| D9OST2002608 | 0 | 0.000 | 2.657 |
| D9OST2003791 | 0 | 0.000 | 7.297 |
| DFNES1000185 | 29.393 | 0 | 0.000 |
| DFNES2000011 | 0 | 0.000 | 4.565 |
| DFNES2000426 | 0 | 0.000 | 15.059 |
| DFNES2000443 | 0 | 0.000 | 0.810 |
| DFNES2001829 | 0 | 0.000 | 11.006 |
| DFNES2006346 | 0 | 0.000 | 8.425 |
| DFNES2007634 | 0 | 0.000 | 7.421 |
| FCBBF1000024 | 0 | 0.000 | 5.237 |
| FCBBF1000027 | 0 | 0.000 | 2.621 |
| FCBBF1000061 | 0 | 0.000 | 2.805 |
| FCBBF1000182 | 0 | 0.000 | 10.114 |
| FCBBF1000197 | 0 | 0.000 | 2.548 |
| FCBBF1000294 | 0 | 0.000 | 1.284 |
| FCBBF1000367 | 0 | 0.000 | 1.812 |
| FCBBF1000412 | 0 | 0.000 | 2.308 |
| FCBBF1000425 | 0 | 0.000 | 6.226 |
| FCBBF1000466 | 14.193 | 0 | 0.760 |
| FCBBF1000574 | 0 | 0.000 | 4.952 |
| FCBBF1000760 | 0 | 0.000 | 0.781 |
| FCBBF2000087 | 0 | 0.000 | 3.441 |
| FCBBF2000591 | 0 | 0.000 | 0.342 |
| FCBBF2000685 | 0 | 0.000 | 8.114 |
| FCBBF2000885 | 0 | 0.000 | 3.661 |
| FCBBF2001001 | 0 | 0.000 | 11.399 |
| FCBBF2001291 | 0 | 0.000 | 2.269 |
| FCBBF2006380 | 0 | 0.000 | 39.231 |
| FCBBF3000115 | 0 | 0.000 | 9.520 |
| FCBBF3000536 | 0 | 0.000 | 3.179 |
| FCBBF3001470 | 0 | 0.000 | 1.619 |
| FCBBF3003800 | 0 | 0.000 | 1.409 |
| FCBBF3004473 | 25.604 | 0 | 1.370 |
| FCBBF3004847 | 0 | 0.000 | 39.231 |
| FCBBF3006821 | 0 | 0.000 | 13.636 |
| FCBBF3007244 | 0 | 0.000 | 9.258 |
| FCBBF3007859 | 0 | 0.000 | 7.748 |
| FCBBF3008153 | 0 | 0.000 | 6.525 |
| FCBBF3010695 | 0 | 0.000 | 2.958 |
| FCBBF3016928 | 0 | 0.000 | 1.045 |
| FCBBF3017396 | 0 | 0.000 | 7.305 |
| FCBBF3017531 | 0 | 0.000 | 1.912 |
| FCBBF3018796 | 0 | 0.000 | 7.405 |
| FCBBF3019714 | 0 | 0.000 | 2.443 |
| FCBBF3019839 | 0 | 0.000 | 17.866 |
| FCBBF3021807 | 0 | 0.000 | 5.171 |
| FCBBF3023667 | 0 | 0.000 | 0.317 |
| FCBBF3024002 | 0 | 0.000 | 6.769 |
| FCBBF3024623 | 0 | 0.000 | 6.682 |
| FCBBF3025528 | 0 | 0.000 | 5.758 |
| FCBBF3025568 | 0 | 0.000 | 19.615 |
| FCBBF3025737 | 0 | 0.000 | 7.752 |
| FCBBF3025905 | 0 | 0.000 | 19.079 |
| FCBBF4000142 | 0 | 0.000 | 4.360 |
| FCBBF4000446 | 0 | 0.000 | 0.896 |
| FCBBF5000261 | 0 | 0.000 | 6.193 |
| FEBRA1000022 | 0 | 0.000 | 12.554 |
| FEBRA1000088 | 0 | 0.000 | 5.090 |
| FEBRA2000105 | 0 | 0.000 | 1.711 |
| FEBRA2000129 | 0 | 0.000 | 3.100 |
| FEBRA2000210 | 0 | 0.000 | 0.843 |
| FEBRA2000462 | 0 | 0.000 | 6.733 |
| FEBRA2000510 | 0 | 0.000 | 4.401 |
| FEBRA2000536 | 0 | 0.000 | 3.757 |
| FEBRA2000581 | 0 | 0.000 | 0.949 |
| FEBRA2000680 | 0 | 0.000 | 33.628 |
| FEBRA2000733 | 3.756 | 0 | 1.206 |
| FEBRA2000740 | 0 | 0.000 | 9.442 |
| FEBRA2000757 | 0 | 0.000 | 3.652 |
| FEBRA2000856 | 0 | 0.000 | 18.931 |
| FEBRA2000880 | 0 | 0.000 | 12.639 |
| FEBRA2001745 | 0 | 0.000 | 1.495 |
| FEBRA2001828 | 0 | 0.000 | 6.489 |
| FEBRA2002527 | 0 | 0.000 | 3.416 |
| FEBRA2003436 | 0 | 0.000 | 6.630 |
| FEBRA2003750 | 0 | 0.000 | 19.596 |
| FEBRA2003833 | 0 | 0.000 | 14.436 |
| FEBRA2003926 | 0 | 0.000 | 4.163 |
| FEBRA2004042 | 47.274 | 0 | 0.000 |
| FEBRA2004412 | 0 | 0.000 | 3.502 |
| FEBRA2004852 | 0 | 0.000 | 32.361 |
| FEBRA2005752 | 0 | 0.000 | 4.964 |
| FEBRA2006092 | 0 | 0.000 | 2.225 |
| FEBRA2007714 | 0 | 0.000 | 9.952 |
| FEBRA2007818 | 0 | 0.000 | 6.669 |
| FEBRA2007901 | 0 | 0.000 | 4.420 |
| FEBRA2008081 | 0 | 0.000 | 0.845 |
| FEBRA2008087 | 3.995 | 0 | 0.214 |
| FEBRA2008210 | 0 | 0.000 | 15.999 |
| FEBRA2008255 | 0 | 0.000 | 6.035 |
| FEBRA2008302 | 0 | 0.000 | 1.269 |
| FEBRA2008583 | 0 | 0.000 | 0.315 |
| FEBRA2009419 | 0 | 0.000 | 1.576 |
| FEBRA2011090 | 0 | 0.000 | 20.700 |
| FEBRA2011392 | 0 | 0.000 | 32.361 |
| FEBRA2014417 | 0 | 0.000 | 1.288 |
| FEBRA2022055 | 0 | 0.000 | 5.712 |
| FEBRA2022504 | 0 | 0.000 | 1.959 |
| FEBRA2023927 | 0 | 0.000 | 4.858 |
| FEBRA2025838 | 0 | 0.000 | 0.980 |
| FEBRA2026984 | 0 | 0.000 | 20.914 |
| FEBRA2028516 | 0 | 0.000 | 1.364 |
| FELIV1000153 | 0 | 0.000 | 0.197 |
| HCASM2000016 | 0 | 0.000 | 1.780 |
| HCASM2000138 | 0 | 0.000 | 10.075 |
| HCASM2000363 | 0 | 0.000 | 0.811 |
| HCASM2001890 | 0 | 0.000 | 15.228 |
| HCASM2008536 | 0 | 0.000 | 15.228 |
| HCHON1000030 | 0 | 0.000 | 0.216 |
| HCHON1000131 | 0 | 0.000 | 9.787 |
| HCHON1000142 | 0 | 0.000 | 15.860 |
| HCHON2000056 | 0 | 0.000 | 1.353 |
| HCHON2000087 | 0 | 58.329 | 0.738 |
| HCHON2000160 | 0 | 0.000 | 1.201 |
| HCHON2000226 | 0 | 0.000 | 0.962 |
| HCHON2000244 | 0 | 0.000 | 1.569 |
| HCHON2000265 | 0 | 0.000 | 2.413 |
| HCHON2000295 | 0 | 0.000 | 1.045 |
| HCHON2000323 | 0 | 0.000 | 2.209 |
| HCHON2000418 | 0 | 0.000 | 15.860 |
| HCHON2000475 | 0 | 0.000 | 3.175 |
| HCHON2000626 | 0 | 0.000 | 5.608 |
| HCHON2000698 | 0 | 0.000 | 10.256 |
| HCHON2000738 | 0 | 0.000 | 3.551 |
| HCHON2000743 | 0 | 0.000 | 7.282 |
| HCHON2000826 | 0 | 0.000 | 0.854 |
| HCHON2001039 | 0 | 0.000 | 2.662 |
| HCHON2001084 | 0 | 0.000 | 2.739 |
| HCHON2001200 | 0 | 40.779 | 1.032 |
| HCHON2001217 | 0 | 0.000 | 4.966 |
| HCHON2001434 | 0 | 0.000 | 3.644 |
| HCHON2001505 | 0 | 0.000 | 16.079 |
| HCHON2001598 | 0 | 0.000 | 5.796 |
| HCHON2001665 | 0 | 0.000 | 1.840 |
| HCHON2001853 | 0 | 0.000 | 6.680 |
| HCHON2002247 | 0 | 0.000 | 3.348 |
| HCHON2004002 | 0 | 0.000 | 0.936 |
| HCHON2005921 | 0 | 0.000 | 24.732 |
| HCHON2006250 | 34.872 | 0 | 0.000 |
| HCHON2007650 | 0 | 0.000 | 7.263 |
| HCHON2008444 | 0 | 0.000 | 15.860 |
| HEART1000149 | 0 | 0.000 | 4.480 |
| HEART1000185 | 0 | 0.000 | 5.657 |
| HEART2000506 | 0 | 0.000 | 4.183 |
| HEART2004931 | 0 | 0.000 | 8.110 |
| HEART2007231 | 0 | 0.000 | 11.555 |
| HHDPC1000114 | 0 | 0.000 | 0.867 |
| HHDPC1000163 | 0 | 0.000 | 1.500 |
| HHDPC2000455 | 0 | 0.000 | 3.932 |
| HHDPC2000572 | 0 | 0.000 | 7.809 |
| HHDPC2000656 | 0 | 0.000 | 10.195 |
| HHDPC2003439 | 0 | 0.000 | 1.517 |
| HHDPC2003983 | 0 | 0.000 | 2.657 |
| HHDPC2007267 | 0 | 0.000 | 0.582 |
| HHDPC2007775 | 0 | 0.000 | 12.888 |
| HLUNG1000055 | 0 | 0.000 | 1.937 |
| HLUNG1000064 | 0 | 0.000 | 8.121 |
| HLUNG1000076 | 0 | 0.000 | 1.003 |
| HLUNG2000004 | 0 | 0.000 | 3.017 |
| HLUNG2000063 | 0 | 0.000 | 5.883 |
| HLUNG2000314 | 0 | 0.000 | 5.852 |
| HLUNG2000412 | 0 | 0.000 | 3.194 |
| HLUNG2000501 | 0 | 0.000 | 1.023 |
| HLUNG2000846 | 0 | 0.000 | 5.388 |
| HLUNG2000884 | 0 | 0.000 | 2.553 |
| HLUNG2001146 | 0 | 0.000 | 6.252 |
| HLUNG2001507 | 0 | 0.000 | 5.972 |
| HLUNG2001518 | 0 | 0.000 | 5.217 |
| HLUNG2001677 | 0 | 0.000 | 4.191 |
| HLUNG2002648 | 0 | 0.000 | 4.311 |
| HLUNG2002811 | 0 | 0.000 | 2.906 |
| HLUNG2003003 | 0 | 0.000 | 28.594 |
| HLUNG2004159 | 0 | 0.000 | 2.162 |
| HLUNG2004170 | 0 | 0.000 | 7.349 |
| HLUNG2004684 | 75.236 | 0 | 0.000 |
| HLUNG2006812 | 0 | 0.000 | 7.053 |
| HLUNG2008333 | 0 | 0.000 | 24.539 |
| HLUNG2008637 | 0 | 0.000 | 2.015 |
| HLUNG2009225 | 0 | 0.000 | 5.396 |
| HLUNG2015184 | 0 | 0.000 | 15.617 |
| HLUNG2017262 | 0 | 0.000 | 3.783 |
| HSYRA1000062 | 0 | 0.000 | 0.511 |
| HSYRA1000152 | 0 | 0.000 | 1.423 |
| HSYRA1000178 | 0 | 0.000 | 2.991 |
| HSYRA2000159 | 0 | 0.000 | 0.719 |
| HSYRA2000224 | 0 | 0.000 | 2.145 |
| HSYRA2000232 | 0 | 0.000 | 0.882 |
| HSYRA2000248 | 0 | 0.000 | 4.053 |
| HSYRA2000255 | 0 | 0.000 | 2.141 |
| HSYRA2000640 | 0 | 0.000 | 4.064 |
| HSYRA2000743 | 0 | 0.000 | 10.153 |
| HSYRA2001105 | 0 | 0.000 | 3.286 |
| HSYRA2001138 | 0 | 0.000 | 0.304 |
| HSYRA2001396 | 0 | 0.000 | 5.436 |
| HSYRA2001476 | 0 | 0.000 | 5.182 |
| HSYRA2001567 | 0 | 0.000 | 2.673 |
| HSYRA2001574 | 0 | 0.000 | 0.897 |
| HSYRA2001580 | 0 | 0.000 | 13.073 |
| HSYRA2001615 | 0 | 0.000 | 2.473 |
| HSYRA2001621 | 0 | 17.301 | 0.000 |
| HSYRA2007650 | 0 | 0.000 | 5.673 |
| IMR321000158 | 0 | 0.000 | 1.816 |
| IMR321000210 | 0 | 0.000 | 1.209 |
| IMR321000219 | 0 | 0.000 | 3.013 |
| IMR321000242 | 0 | 0.000 | 6.707 |
| IMR321000266 | 0 | 0.000 | 3.375 |
| IMR322000121 | 0 | 0.000 | 1.330 |
| IMR322000730 | 3.055 | 0 | 0.327 |
| IMR322000935 | 0 | 0.000 | 1.786 |
| IMR322001049 | 0 | 0.000 | 6.909 |
| IMR322001435 | 0 | 0.000 | 3.361 |
| IMR322001534 | 0 | 0.000 | 9.855 |
| IMR322001600 | 0 | 15.181 | 0.960 |
| IMR322001710 | 0 | 0.000 | 2.313 |
| IMR322001879 | 0 | 0.000 | 11.495 |
| IMR322019070 | 0 | 0.000 | 3.191 |
| KIDNE1000036 | 0 | 0.000 | 25.490 |
| KIDNE1000145 | 0 | 0.000 | 16.491 |
| KIDNE2000085 | 0 | 0.000 | 3.679 |
| KIDNE2000330 | 76.084 | 0 | 0.000 |
| KIDNE2000403 | 0 | 0.000 | 1.971 |
| KIDNE2001162 | 0 | 0.000 | 1.670 |
| KIDNE2001467 | 0 | 0.000 | 0.933 |
| KIDNE2001979 | 0 | 0.000 | 7.027 |
| KIDNE2002252 | 0 | 0.000 | 2.600 |
| KIDNE2003305 | 0 | 0.000 | 1.004 |
| KIDNE2003941 | 0 | 0.000 | 0.602 |
| KIDNE2005321 | 0 | 0.000 | 11.785 |
| KIDNE2005676 | 0 | 0.000 | 5.034 |
| KIDNE2006014 | 0 | 0.000 | 3.541 |
| KIDNE2006030 | 0 | 0.000 | 7.636 |
| KIDNE2006248 | 0 | 0.000 | 1.112 |
| KIDNE2006465 | 0 | 0.000 | 3.690 |
| KIDNE2007040 | 0 | 0.000 | 25.490 |
| KIDNE2007328 | 0 | 0.000 | 2.110 |
| KIDNE2007352 | 0 | 0.000 | 0.992 |
| KIDNE2007811 | 0 | 0.000 | 10.218 |
| KIDNE2007954 | 0 | 0.000 | 2.514 |
| KIDNE2008048 | 0 | 0.000 | 3.839 |
| KIDNE2009367 | 0 | 0.000 | 9.888 |
| KIDNE2009605 | 0 | 0.000 | 18.012 |
| KIDNE2010049 | 76.623 | 0 | 4.101 |
| KIDNE2012784 | 0 | 0.000 | 40.625 |
| KIDNE2013158 | 0 | 0.000 | 21.635 |
| KIDNE2013845 | 0 | 0.000 | 2.666 |
| KIDNE2014290 | 0 | 0.000 | 3.345 |
| KIDNE2014325 | 0 | 0.000 | 4.512 |
| KIDNE2017040 | 0 | 0.000 | 4.011 |
| LIVER1000079 | 0 | 0.000 | 2.090 |
| LIVER1000132 | 0 | 0.000 | 0.597 |
| LIVER2000033 | 0 | 0.000 | 1.650 |
| LIVER2001539 | 0 | 0.000 | 5.939 |
| LIVER2001608 | 29.267 | 0 | 1.566 |
| LIVER2002842 | 0 | 0.000 | 9.107 |
| LIVER2003234 | 0 | 0.000 | 12.095 |
| LIVER2005218 | 0 | 0.000 | 5.883 |
| LIVER2007783 | 0 | 0.000 | 4.894 |
| LIVER2008465 | 0 | 0.000 | 7.893 |
| MAMGL1000083 | 0 | 0.000 | 0.346 |
| MESAN2000264 | 0 | 0.000 | 2.350 |
| MESAN2000267 | 0 | 0.000 | 2.930 |
| MESAN2000894 | 0 | 0.000 | 24.337 |
| MESAN2001154 | 0 | 0.000 | 1.589 |
| MESAN2002086 | 0 | 0.000 | 2.482 |
| MESAN2002424 | 0 | 0.000 | 24.337 |
| MESAN2002724 | 0 | 0.000 | 5.499 |
| MESAN2002844 | 0 | 0.000 | 8.642 |
| MESAN2003039 | 0 | 0.000 | 19.415 |
| MESAN2003058 | 0 | 0.000 | 7.143 |
| MESAN2003444 | 0 | 0.000 | 8.886 |
| MESAN2003709 | 0 | 0.000 | 9.892 |
| MESAN2005303 | 0 | 0.000 | 9.449 |
| MESAN2005957 | 0 | 0.000 | 17.666 |
| MESAN2006043 | 0 | 0.000 | 9.499 |
| MESAN2006580 | 0 | 0.000 | 24.337 |
| MESAN2006743 | 0 | 0.000 | 13.792 |
| MESAN2009580 | 0 | 0.000 | 1.325 |
| MESAN2013284 | 0 | 0.000 | 2.930 |
| MESAN2014295 | 0 | 0.000 | 24.337 |
| MESAN2016552 | 0 | 0.000 | 14.511 |
| MESAN2017152 | 0 | 0.000 | 24.337 |
| NB9N41000121 | 16.735 | 0 | 0.000 |
| NB9N42000281 | 0 | 0.000 | 2.280 |
| NOVAR1000015 | 0 | 0.000 | 3.622 |
| NOVAR1000091 | 0 | 0.000 | 2.310 |
| NOVAR2000710 | 0 | 0.000 | 4.201 |
| NT2NE1000163 | 0 | 0.000 | 2.899 |
| NT2NE2000056 | 8.755 | 0 | 3.280 |
| NT2NE2000327 | 0 | 0.000 | 3.000 |
| NT2NE2000383 | 0 | 0.000 | 5.437 |
| NT2NE2000384 | 0 | 0.000 | 2.745 |
| NT2NE2000455 | 0 | 0.000 | 24.761 |
| NT2NE2000575 | 0 | 0.000 | 1.720 |
| NT2NE2000707 | 0 | 0.000 | 1.596 |
| NT2NE2000963 | 5.112 | 0 | 0.547 |
| NT2NE2001000 | 0 | 0.000 | 2.053 |
| NT2NE2001176 | 0 | 0.000 | 1.362 |
| NT2NE2001337 | 0 | 0.000 | 5.420 |
| NT2NE2001435 | 0 | 0.000 | 5.072 |
| NT2NE2001545 | 0 | 0.000 | 2.137 |
| NT2NE2001617 | 0 | 0.000 | 5.411 |
| NT2NE2001660 | 0 | 0.000 | 9.341 |
| NT2NE2001793 | 0 | 0.000 | 7.422 |
| NT2NE2002620 | 0 | 90.183 | 0.000 |
| NT2NE2003185 | 0 | 0.000 | 4.521 |
| NT2NE2003485 | 0 | 0.000 | 5.552 |
| NT2NE2003569 | 0 | 0.000 | 2.070 |
| NT2NE2004519 | 0 | 0.000 | 2.137 |
| NT2NE2004716 | 0 | 0.000 | 4.578 |
| NT2NE2005358 | 0 | 0.000 | 24.761 |
| NT2NE2006458 | 0 | 0.000 | 3.372 |
| NT2NE2006659 | 0 | 0.000 | 21.581 |
| NT2NE2009295 | 0 | 0.000 | 2.379 |
| NT2NE2015974 | 0 | 0.000 | 9.111 |
| NT2NE2017492 | 0 | 0.000 | 20.127 |
| NT2RI1000127 | 0 | 0.000 | 2.165 |
| NT2RI2000007 | 0 | 0.000 | 39.625 |
| NT2RI2000064 | 0 | 0.000 | 3.210 |
| NT2RI2000282 | 0 | 0.000 | 0.378 |
| NT2RI2000294 | 0 | 0.000 | 2.257 |
| NT2RI2000344 | 0 | 76.334 | 0.000 |
| NT2RI2000578 | 7.452 | 31.528 | 0.399 |
| NT2RI2000671 | 0 | 0.000 | 1.255 |
| NT2RI2000685 | 0 | 0.000 | 3.545 |
| NT2RI2000689 | 34.460 | 0 | 1.844 |
| NT2RI2000727 | 0 | 0.000 | 0.617 |
| NT2RI2001091 | 0 | 0.000 | 4.193 |
| NT2RI2001230 | 0 | 0.000 | 0.920 |
| NT2RI2001540 | 1.269 | 0 | 1.494 |
| NT2RI2001657 | 0 | 0.000 | 1.914 |
| NT2RI2001859 | 0 | 0.000 | 1.157 |
| NT2RI2002091 | 0.897 | 2.529 | 0.976 |
| NT2RI2002152 | 0 | 0.000 | 3.000 |
| NT2RI2002316 | 0 | 0.000 | 0.956 |
| NT2RI2002391 | 18.073 | 0 | 0.000 |
| NT2RI2002654 | 13.092 | 0 | 0.000 |
| NT2RI2002865 | 0 | 0.000 | 8.087 |
| NT2RI2003304 | 5.848 | 0 | 0.000 |
| NT2RI2003317 | 0 | 0.000 | 3.760 |
| NT2RI2003338 | 0 | 0.000 | 4.831 |
| NT2RI2003344 | 0 | 0.000 | 6.968 |
| NT2RI2003407 | 0 | 0.000 | 1.444 |
| NT2RI2003420 | 0 | 0.000 | 1.837 |
| NT2RI2003556 | 0 | 0.000 | 0.908 |
| NT2RI2003695 | 0 | 0.000 | 0.939 |
| NT2RI2003738 | 0 | 0.000 | 1.252 |
| NT2RI2003751 | 0 | 0.000 | 1.652 |
| NT2RI2004157 | 0 | 0.000 | 39.625 |
| NT2RI2004188 | 0 | 0.000 | 3.425 |
| NT2RI2004230 | 0 | 0.000 | 6.843 |
| NT2RI2004535 | 0 | 0.000 | 2.283 |
| NT2RI2005116 | 0 | 0.000 | 1.525 |
| NT2RI2005335 | 0 | 0.000 | 3.232 |
| NT2RI2005358 | 0 | 0.000 | 24.478 |
| NT2RI2005405 | 0 | 0.000 | 1.004 |
| NT2RI2005713 | 0 | 0.000 | 1.764 |
| NT2RI2005723 | 11.184 | 0 | 1.197 |
| NT2RI2005811 | 0 | 0.000 | 1.827 |
| NT2RI2005814 | 0 | 0.000 | 1.553 |
| NT2RI2005818 | 0 | 0.000 | 3.038 |
| NT2RI2006412 | 0 | 0.000 | 0.978 |
| NT2RI2006825 | 0 | 0.000 | 3.270 |
| NT2RI2007054 | 0 | 0.000 | 3.097 |
| NT2RI2007148 | 0 | 0.000 | 2.253 |
| NT2RI2007277 | 0 | 0.000 | 1.169 |
| NT2RI2007384 | 9.651 | 0 | 1.033 |
| NT2RI2007445 | 0 | 0.000 | 2.139 |
| NT2RI2007498 | 0 | 0.000 | 0.486 |
| NT2RI2007589 | 23.800 | 0 | 0.000 |
| NT2RI2007629 | 0 | 0.000 | 2.404 |
| NT2RI2007987 | 0 | 0.000 | 2.147 |
| NT2RI2008007 | 0 | 0.000 | 23.084 |
| NT2RI2008045 | 0 | 0.000 | 0.926 |
| NT2RI2008336 | 0 | 0.000 | 0.956 |
| NT2RI2008481 | 0 | 0.000 | 2.646 |
| NT2RI2008526 | 17.086 | 0 | 0.000 |
| NT2RI2008724 | 0 | 0.000 | 2.190 |
| NT2RI2008749 | 0 | 0.000 | 2.023 |
| NT2RI2008791 | 0 | 0.000 | 1.392 |
| NT2RI2008942 | 0 | 0.000 | 16.259 |
| NT2RI2009037 | 0 | 0.000 | 1.005 |
| NT2RI2009065 | 10.408 | 0 | 0.000 |
| NT2RI2009269 | 0 | 0.000 | 1.179 |
| NT2RI2009402 | 0 | 0.000 | 7.482 |
| NT2RI2009406 | 0 | 0.000 | 3.356 |
| NT2RI2009583 | 0 | 0.000 | 0.267 |
| NT2RI2014247 | 0 | 0.000 | 18.191 |
| NT2RI2014733 | 0 | 0.000 | 29.743 |
| NT2RI2017529 | 0 | 0.000 | 1.157 |
| NT2RI2022468 | 0 | 0.000 | 7.995 |
| NT2RI2024313 | 0 | 0.000 | 17.475 |
| NT2RI2027323 | 0 | 0.000 | 39.625 |
| NT2RI3001445 | 0 | 0.000 | 7.554 |
| NT2RI3005724 | 0 | 0.000 | 7.069 |
| NT2RI3005923 | 0 | 0.000 | 23.249 |
| NT2RI3006284 | 46.111 | 0 | 0.000 |
| NT2RI3006340 | 0 | 0.000 | 19.479 |
| NT2RI3006666 | 0 | 0.000 | 24.901 |
| NT2RI3006673 | 0 | 0.000 | 11.902 |
| NT2RI3007291 | 0 | 0.000 | 14.928 |
| NT2RI3007543 | 0 | 0.000 | 1.012 |
| NT2RP6000017 | 0 | 0.000 | 3.569 |
| NT2RP6000039 | 0 | 0.000 | 4.277 |
| NT2RP6000059 | 0 | 0.000 | 25.869 |
| NT2RP6000077 | 0 | 0.000 | 30.666 |
| NT2RP6000078 | 0 | 0.000 | 0.429 |
| NT2RP6000085 | 0 | 0.000 | 11.032 |
| NT2RP6000123 | 0 | 0.000 | 0.467 |
| NT2RP7000069 | 0 | 0.000 | 8.035 |
| NT2RP7000076 | 0 | 0.000 | 0.727 |
| NT2RP7000173 | 0 | 0.000 | 0.663 |
| NT2RP7000477 | 0 | 0.000 | 2.360 |
| NT2RP7000586 | 0 | 0.000 | 13.104 |
| NT2RP7000600 | 0 | 0.000 | 7.929 |
| NT2RP7000906 | 0 | 0.000 | 3.112 |
| NT2RP7001166 | 0 | 0.000 | 2.676 |
| NT2RP7001962 | 0 | 0.000 | 3.168 |
| NT2RP7002028 | 0 | 0.000 | 7.190 |
| NT2RP7002376 | 0 | 0.000 | 14.471 |
| NT2RP7002379 | 0 | 0.000 | 2.402 |
| NT2RP7002449 | 0 | 0.000 | 2.409 |
| NT2RP7002802 | 0 | 0.000 | 8.717 |
| NT2RP7002875 | 0 | 0.000 | 1.101 |
| NT2RP7002982 | 0 | 0.000 | 9.273 |
| NT2RP7003055 | 0 | 0.000 | 12.585 |
| NT2RP7003203 | 0 | 0.000 | 4.406 |
| NT2RP7003319 | 0 | 0.000 | 31.557 |
| NT2RP7003632 | 0 | 0.000 | 1.942 |
| NT2RP7003647 | 0 | 0.000 | 5.615 |
| NT2RP7003688 | 0 | 0.000 | 0.881 |
| NT2RP7004114 | 0 | 0.000 | 6.650 |
| NT2RP7004123 | 0 | 0.000 | 2.289 |
| NT2RP7004196 | 0 | 0.000 | 1.563 |
| NT2RP7004348 | 0 | 0.000 | 2.695 |
| NT2RP7004352 | 0 | 0.000 | 6.077 |
| NT2RP7004373 | 0 | 0.000 | 2.807 |
| NT2RP7004559 | 0 | 0.000 | 10.166 |
| NT2RP7004728 | 0 | 0.000 | 1.875 |
| NT2RP7004751 | 0 | 44.129 | 1.116 |
| NT2RP7004915 | 0 | 0.000 | 17.708 |
| NT2RP7004925 | 0 | 0.000 | 1.506 |
| NT2RP7004946 | 0 | 0.000 | 2.053 |
| NT2RP7005513 | 0 | 0.000 | 2.016 |
| NT2RP7005529 | 0 | 0.000 | 8.806 |
| NT2RP7005675 | 0 | 0.000 | 10.406 |
| NT2RP7005750 | 0 | 0.000 | 4.268 |
| NT2RP7006223 | 0 | 0.000 | 5.039 |
| NT2RP7006490 | 0 | 0.000 | 2.560 |
| NT2RP7006539 | 0 | 0.000 | 10.410 |
| NT2RP7006619 | 0 | 0.000 | 2.633 |
| NT2RP7006717 | 0 | 0.000 | 5.493 |
| NT2RP7006886 | 0 | 0.000 | 11.344 |
| NT2RP7007114 | 0 | 0.000 | 19.540 |
| NT2RP7007154 | 0 | 0.000 | 1.360 |
| NT2RP7007252 | 0 | 0.000 | 8.190 |
| NT2RP7007310 | 82.993 | 0 | 0.000 |
| NT2RP7007381 | 0 | 0.000 | 18.704 |
| NT2RP7007537 | 27.403 | 0 | 0.000 |
| NT2RP7007610 | 0 | 0.000 | 3.452 |
| NT2RP7008161 | 0 | 0.000 | 7.488 |
| NT2RP7008167 | 0 | 0.000 | 2.165 |
| NT2RP7008190 | 0 | 0.000 | 1.582 |
| NT2RP7008315 | 0 | 0.000 | 8.950 |
| NT2RP7008543 | 0 | 0.000 | 3.281 |
| NT2RP7009087 | 0 | 0.000 | 2.897 |
| NT2RP7009097 | 40.067 | 0 | 0.000 |
| NT2RP7009147 | 0 | 0.000 | 1.900 |
| NT2RP7009259 | 0 | 0.000 | 2.010 |
| NT2RP7009482 | 0 | 0.000 | 5.527 |
| NT2RP7010612 | 0 | 0.000 | 25.799 |
| NT2RP7013573 | 0 | 0.000 | 8.594 |
| NT2RP7017474 | 0 | 0.000 | 22.450 |
| NTONG1000033 | 0 | 0.000 | 1.461 |
| NTONG1000098 | 0 | 0.000 | 0.760 |
| NTONG1000182 | 0 | 0.000 | 1.477 |
| NTONG1000246 | 0 | 0.000 | 1.865 |
| NTONG2000265 | 0 | 0.000 | 1.017 |
| NTONG2000876 | 0 | 0.000 | 1.661 |
| NTONG2001550 | 0 | 0.000 | 4.151 |
| NTONG2001567 | 0 | 0.000 | 1.097 |
| NTONG2002582 | 0 | 0.000 | 13.457 |
| NTONG2003454 | 0 | 0.000 | 24.889 |
| NTONG2003839 | 0 | 0.000 | 6.254 |
| NTONG2004844 | 0 | 0.000 | 1.933 |
| NTONG2005363 | 0 | 0.000 | 13.929 |
| NTONG2005391 | 0 | 0.000 | 13.902 |
| NTONG2005520 | 0 | 0.000 | 0.794 |
| NTONG2005801 | 0 | 0.000 | 4.250 |
| NTONG2005897 | 0 | 0.000 | 8.187 |
| NTONG2008944 | 0 | 0.000 | 6.965 |
| OCBBF1000086 | 0 | 0.000 | 1.826 |
| OCBBF1000175 | 0 | 0.000 | 8.591 |
| OCBBF2000015 | 17.632 | 0 | 3.774 |
| OCBBF2000467 | 0 | 0.000 | 2.406 |
| OCBBF2000677 | 0 | 0.000 | 19.428 |
| OCBBF2000986 | 0 | 0.000 | 1.149 |
| OCBBF2001166 | 0 | 0.000 | 6.537 |
| OCBBF2001210 | 0 | 0.000 | 4.324 |
| OCBBF2001323 | 0 | 0.000 | 10.255 |
| OCBBF2001586 | 0 | 0.000 | 8.553 |
| OCBBF2001961 | 0 | 0.000 | 48.981 |
| OCBBF2002086 | 0 | 0.000 | 15.370 |
| OCBBF2002656 | 0 | 0.000 | 1.973 |
| OCBBF2003246 | 0 | 0.000 | 11.672 |
| OCBBF2003925 | 0 | 0.000 | 0.994 |
| OCBBF2004273 | 19.976 | 0 | 0.000 |
| OCBBF2004757 | 0 | 0.000 | 2.956 |
| OCBBF2005476 | 0 | 0.000 | 16.727 |
| OCBBF2005956 | 0 | 0.000 | 3.386 |
| OCBBF2006313 | 0 | 0.000 | 6.810 |
| OCBBF2006849 | 0 | 0.000 | 2.410 |
| OCBBF2007028 | 0 | 0.000 | 8.234 |
| OCBBF2007415 | 0 | 0.000 | 0.955 |
| OCBBF2007829 | 0 | 0.000 | 7.203 |
| OCBBF2008005 | 0 | 0.000 | 3.787 |
| OCBBF2008144 | 0 | 0.000 | 3.440 |
| OCBBF2009242 | 0 | 0.000 | 3.377 |
| OCBBF2009301 | 46.196 | 0 | 0.000 |
| OCBBF2009424 | 0 | 0.000 | 8.655 |
| OCBBF2009772 | 0 | 0.000 | 1.404 |
| OCBBF2010313 | 0 | 0.000 | 16.359 |
| OCBBF2010557 | 0 | 0.000 | 43.708 |
| OCBBF2010830 | 0 | 0.000 | 2.099 |
| OCBBF2010945 | 0 | 0.000 | 48.981 |
| OCBBF2011021 | 0 | 0.000 | 1.724 |
| OCBBF2011228 | 0 | 0.000 | 25.500 |
| OCBBF2011232 | 0 | 0.000 | 9.556 |
| OCBBF2011536 | 0 | 0.000 | 2.379 |
| OCBBF2011759 | 0 | 0.000 | 3.344 |
| OCBBF2013149 | 0 | 0.000 | 1.850 |
| OCBBF2013843 | 0 | 50.379 | 0.637 |
| OCBBF2014064 | 0 | 0.000 | 3.258 |
| OCBBF2014292 | 0 | 0.000 | 3.706 |
| OCBBF2017398 | 0 | 0.000 | 3.068 |
| OCBBF2017754 | 0 | 0.000 | 8.956 |
| OCBBF2017791 | 0 | 0.000 | 9.166 |
| OCBBF2017882 | 0 | 0.000 | 8.510 |
| OCBBF2018012 | 0 | 0.000 | 10.043 |
| OCBBF2018563 | 0 | 0.000 | 24.528 |
| OCBBF2018873 | 0 | 0.000 | 6.611 |
| OCBBF2018956 | 0 | 0.000 | 2.019 |
| OCBBF2020741 | 0 | 0.000 | 33.655 |
| OCBBF2021833 | 0 | 0.000 | 17.297 |
| OCBBF2024850 | 0 | 0.000 | 26.505 |
| OCBBF2026368 | 0 | 0.000 | 19.710 |
| OCBBF2027148 | 0 | 0.000 | 27.753 |
| OCBBF2036743 | 0 | 0.000 | 13.183 |
| OCBBF3001333 | 0 | 0.000 | 3.832 |
| OCBBF3004487 | 0 | 0.000 | 5.404 |
| PEBLM1000144 | 0 | 0.000 | 7.153 |
| PEBLM2000030 | 0 | 0.000 | 4.390 |
| PEBLM2000147 | 0 | 0.000 | 1.522 |
| PEBLM2000326 | 0 | 0.000 | 6.474 |
| PEBLM2004452 | 0 | 0.000 | 6.439 |
| PEBLM2006366 | 0 | 0.000 | 3.301 |
| PEBLM2006709 | 0 | 0.000 | 6.636 |
| PERIC2006443 | 0 | 0.000 | 5.792 |
| PERIC2007068 | 0 | 0.000 | 5.521 |
| PLACE5000013 | 0 | 0.000 | 9.466 |
| PLACE5000113 | 0 | 0.000 | 10.260 |
| PLACE5000159 | 0 | 0.000 | 8.132 |
| PLACE5000170 | 0 | 0.000 | 4.147 |
| PLACE5000372 | 0 | 0.000 | 40.126 |
| PLACE6000145 | 23.096 | 0 | 0.000 |
| PLACE6000263 | 0 | 0.000 | 1.813 |
| PLACE6000379 | 0 | 0.000 | 1.264 |
| PLACE6000414 | 0 | 0.000 | 6.113 |
| PLACE6001064 | 0 | 0.000 | 3.421 |
| PLACE6001185 | 0 | 0.000 | 25.473 |
| PLACE6001281 | 0 | 0.000 | 4.106 |
| PLACE6001443 | 0 | 0.000 | 11.713 |
| PLACE6001886 | 0 | 0.000 | 40.398 |
| PLACE6002084 | 0 | 0.000 | 0.881 |
| PLACE6002102 | 0 | 0.000 | 6.738 |
| PLACE6002668 | 0 | 0.000 | 6.437 |
| PLACE6002960 | 0 | 0.000 | 2.647 |
| PLACE6003218 | 0 | 0.000 | 17.355 |
| PLACE6003383 | 0 | 0.000 | 1.424 |
| PLACE6004005 | 0 | 0.000 | 1.178 |
| PLACE6004312 | 0 | 0.000 | 0.719 |
| PLACE6004397 | 0 | 0.000 | 2.192 |
| PLACE6004491 | 0 | 0.000 | 40.126 |
| PLACE6004738 | 0 | 0.000 | 25.098 |
| PLACE6005482 | 0 | 0.000 | 22.076 |
| PLACE6006042 | 0 | 0.000 | 6.493 |
| PLACE6006137 | 0 | 0.000 | 4.074 |
| PLACE6006186 | 0 | 0.000 | 25.880 |
| PLACE6006549 | 0 | 0.000 | 1.905 |
| PLACE6008775 | 0 | 0.000 | 6.595 |
| PLACE6008824 | 0 | 0.000 | 40.126 |
| PLACE6009338 | 0 | 0.000 | 29.967 |
| PLACE6010077 | 0 | 0.000 | 2.077 |
| PLACE6011057 | 0 | 0.000 | 2.688 |
| PLACE6012942 | 0 | 0.000 | 40.126 |
| PLACE6015731 | 0 | 0.000 | 18.307 |
| PLACE6016383 | 0 | 0.000 | 7.710 |
| PLACE6017431 | 0 | 0.000 | 40.126 |
| PLACE6018938 | 0 | 0.000 | 18.301 |
| PLACE7006240 | 0 | 0.000 | 30.024 |
| PROST1000097 | 0 | 0.000 | 7.013 |
| PROST1000199 | 16.283 | 0 | 4.357 |
| PROST1000220 | 0 | 0.000 | 0.810 |
| PROST1000226 | 0 | 0.000 | 2.134 |
| PROST1000246 | 0 | 0.000 | 13.104 |
| PROST1000526 | 0 | 0.000 | 3.013 |
| PROST1000564 | 0 | 0.000 | 1.603 |
| PROST2000138 | 5.795 | 0 | 5.479 |
| PROST2000273 | 0 | 0.000 | 25.167 |
| PROST2002651 | 0 | 0.000 | 7.133 |
| PROST2003232 | 0 | 0.000 | 0.814 |
| PROST2003338 | 0 | 0.000 | 4.005 |
| PROST2003396 | 0 | 0.000 | 7.125 |
| PROST2004095 | 0 | 0.000 | 1.116 |
| PROST2004258 | 0 | 0.000 | 9.101 |
| PROST2004270 | 0 | 0.000 | 2.104 |
| PROST2004416 | 0 | 0.000 | 0.811 |
| PROST2005285 | 0 | 0.000 | 8.902 |
| PROST2005604 | 0 | 0.000 | 7.420 |
| PROST2006030 | 0 | 0.000 | 4.513 |
| PROST2006536 | 0 | 0.000 | 2.647 |
| PROST2006737 | 0 | 0.000 | 8.672 |
| PROST2007200 | 0 | 0.000 | 14.088 |
| PROST2008271 | 0 | 0.000 | 4.674 |
| PROST2008489 | 14.433 | 0 | 0.772 |
| PROST2008770 | 0 | 0.000 | 0.930 |
| PROST2010318 | 0 | 0.000 | 14.395 |
| PROST2010545 | 0 | 0.000 | 15.696 |
| PROST2010782 | 0 | 0.000 | 0.290 |
| PROST2012007 | 0 | 0.000 | 1.354 |
| PROST2012448 | 0 | 0.000 | 9.731 |
| PROST2012890 | 0 | 0.000 | 18.191 |
| PROST2014916 | 0 | 0.000 | 4.345 |
| PROST2015924 | 0 | 0.000 | 8.082 |
| PROST2016918 | 0 | 0.000 | 15.159 |
| PROST2017203 | 0 | 0.000 | 1.549 |
| PROST2017617 | 0 | 0.000 | 25.167 |
| PROST2017692 | 0 | 0.000 | 0.879 |
| PROST2018977 | 0 | 0.000 | 15.499 |
| PUAEN1000057 | 0 | 0.000 | 7.346 |
| PUAEN1000065 | 0 | 0.000 | 1.598 |
| PUAEN1000164 | 0 | 0.000 | 0.475 |
| PUAEN1000239 | 0 | 0.000 | 3.697 |
| PUAEN2000374 | 0 | 0.000 | 1.145 |
| PUAEN2001586 | 0 | 0.000 | 3.406 |
| PUAEN2007044 | 0 | 0.000 | 1.710 |
| PUAEN2008228 | 0 | 0.000 | 17.449 |
| PUAEN2009655 | 0 | 0.000 | 4.359 |
| SALGL1000157 | 0 | 0.000 | 0.465 |
| SKMUS1000014 | 0 | 0.000 | 8.773 |
| SKMUS1000022 | 0 | 0.000 | 0.496 |
| SKMUS1000084 | 0 | 0.000 | 3.674 |
| SKMUS1000138 | 0 | 0.000 | 0.344 |
| SKMUS1000177 | 0 | 0.000 | 14.082 |
| SKMUS1000186 | 0 | 0.000 | 0.479 |
| SKMUS2000020 | 0 | 0.000 | 4.393 |
| SKMUS2000343 | 0 | 0.000 | 18.379 |
| SKMUS2000361 | 0 | 0.000 | 1.275 |
| SKMUS2000380 | 0 | 0.000 | 2.586 |
| SKMUS2000873 | 0 | 0.000 | 1.312 |
| SKMUS2000902 | 0 | 0.000 | 12.901 |
| SKMUS2000931 | 0 | 0.000 | 0.649 |
| SKMUS2000954 | 0 | 0.000 | 2.027 |
| SKMUS2001129 | 0 | 0.000 | 12.130 |
| SKMUS2001454 | 7.522 | 0 | 0.403 |
| SKMUS2001501 | 0 | 0.000 | 2.600 |
| SKNMC1000110 | 0 | 0.000 | 4.318 |
| SKNMC1000137 | 0 | 0.000 | 1.264 |
| SKNMC1000168 | 0 | 0.000 | 1.997 |
| SKNMC1000251 | 0 | 0.000 | 8.366 |
| SKNMC2000065 | 0 | 0.000 | 2.575 |
| SKNMC2000097 | 0 | 0.000 | 1.063 |
| SKNMC2000305 | 0 | 0.000 | 4.201 |
| SKNMC2000635 | 0 | 0.000 | 0.856 |
| SKNMC2000649 | 0 | 0.000 | 2.176 |
| SKNMC2000877 | 0 | 0.000 | 2.827 |
| SKNSH1000086 | 0 | 0.000 | 10.184 |
| SKNSH2000347 | 0 | 0.000 | 8.028 |
| SKNSH2000482 | 0 | 0.000 | 23.251 |
| SKNSH2000819 | 0 | 0.000 | 5.937 |
| SKNSH2002325 | 0 | 0.000 | 4.553 |
| SKNSH2003528 | 0 | 0.000 | 3.621 |
| SKNSH2008043 | 0 | 0.000 | 12.076 |
| SMINT1000042 | 59.707 | 0 | 6.391 |
| SMINT1000100 | 0 | 0.000 | 6.640 |
| SMINT1000117 | 0 | 0.000 | 1.569 |
| SMINT1000131 | 0 | 0.000 | 3.277 |
| SMINT2000159 | 0 | 0.000 | 6.518 |
| SMINT2000277 | 0 | 0.000 | 2.162 |
| SMINT2000396 | 0 | 0.000 | 5.840 |
| SMINT2000441 | 0 | 0.000 | 1.615 |
| SMINT2000468 | 0 | 0.000 | 5.458 |
| SMINT2000545 | 0 | 0.000 | 2.827 |
| SMINT2000629 | 0 | 0.000 | 3.203 |
| SMINT2001950 | 0 | 0.000 | 12.743 |
| SMINT2002159 | 0 | 0.000 | 8.177 |
| SMINT2002314 | 53.944 | 0 | 0.000 |
| SMINT2002689 | 0 | 0.000 | 51.875 |
| SMINT2002778 | 0 | 0.000 | 25.261 |
| SMINT2002976 | 0 | 0.000 | 6.221 |
| SMINT2003169 | 0 | 0.000 | 0.561 |
| SMINT2004589 | 0 | 0.000 | 4.780 |
| SMINT2005368 | 0 | 0.000 | 1.401 |
| SMINT2005956 | 0 | 0.000 | 6.278 |
| SMINT2006801 | 0 | 0.000 | 19.209 |
| SMINT2007187 | 0 | 0.000 | 8.007 |
| SMINT2008960 | 0 | 0.000 | 0.816 |
| SMINT2010959 | 0 | 0.000 | 18.459 |
| SMINT2011033 | 0 | 0.000 | 25.261 |
| SMINT2012122 | 0 | 0.000 | 4.169 |
| SMINT2013031 | 0 | 0.000 | 7.403 |
| SMINT2013695 | 0 | 0.000 | 25.261 |
| SMINT2014480 | 35.406 | 0 | 0.000 |
| SMINT2018681 | 0 | 0.000 | 4.988 |
| SPLEN1000106 | 0 | 0.000 | 13.880 |
| SPLEN1000116 | 0 | 0.000 | 0.590 |
| SPLEN1000166 | 0 | 0.000 | 2.696 |
| SPLEN2000243 | 0 | 0.000 | 25.928 |
| SPLEN2000255 | 0 | 0.000 | 5.495 |
| SPLEN2000505 | 0 | 0.000 | 0.503 |
| SPLEN2000839 | 0 | 0.000 | 10.886 |
| SPLEN2001141 | 0 | 0.000 | 2.253 |
| SPLEN2001157 | 0 | 0.000 | 3.280 |
| SPLEN2001689 | 0 | 0.000 | 16.646 |
| SPLEN2002335 | 0 | 0.000 | 6.038 |
| SPLEN2002463 | 0 | 0.000 | 9.032 |
| SPLEN2003297 | 0 | 0.000 | 31.668 |
| SPLEN2004220 | 0 | 0.000 | 3.430 |
| SPLEN2005009 | 0 | 0.000 | 10.253 |
| SPLEN2005429 | 0 | 0.000 | 17.478 |
| SPLEN2005927 | 0 | 0.000 | 5.136 |
| SPLEN2006122 | 18.769 | 0 | 0.000 |
| SPLEN2006133 | 21.141 | 0 | 2.263 |
| SPLEN2006143 | 0 | 0.000 | 2.668 |
| SPLEN2006232 | 0 | 0.000 | 3.421 |
| SPLEN2006305 | 0 | 0.000 | 1.507 |
| SPLEN2006374 | 0 | 0.000 | 5.438 |
| SPLEN2007388 | 0 | 0.000 | 31.668 |
| SPLEN2007498 | 0 | 0.000 | 3.121 |
| SPLEN2009315 | 0 | 0.000 | 40.674 |
| SPLEN2010195 | 0 | 0.000 | 4.164 |
| SPLEN2011419 | 0 | 0.000 | 2.850 |
| SPLEN2011737 | 0 | 0.000 | 3.107 |
| SPLEN2012571 | 0 | 0.000 | 3.792 |
| SPLEN2012800 | 0 | 0.000 | 0.407 |
| SPLEN2013690 | 0 | 0.000 | 12.880 |
| SPLEN2014080 | 0 | 0.000 | 5.651 |
| SPLEN2014572 | 0 | 0.000 | 7.423 |
| SPLEN2016268 | 9.628 | 0 | 0.000 |
| SPLEN2016421 | 0 | 0.000 | 2.471 |
| SPLEN2016554 | 0 | 0.000 | 22.228 |
| SPLEN2016972 | 0 | 0.000 | 4.837 |
| SPLEN2017318 | 0 | 0.000 | 6.052 |
| SPLEN2017918 | 0 | 0.000 | 3.262 |
| SPLEN2018098 | 0 | 0.000 | 40.674 |
| SPLEN2018157 | 0 | 0.000 | 5.754 |
| SPLEN2019169 | 0 | 0.000 | 6.752 |
| SPLEN2019311 | 0 | 0.000 | 57.827 |
| SPLEN2019405 | 0 | 0.000 | 3.416 |
| SPLEN2021194 | 0 | 0.000 | 8.047 |
| SPLEN2021273 | 0 | 0.000 | 17.589 |
| SPLEN2022522 | 0 | 0.000 | 1.691 |
| SPLEN2027113 | 0 | 0.000 | 1.952 |
| SPLEN2028466 | 0 | 0.000 | 0.987 |
| SPLEN2031547 | 0 | 0.000 | 5.633 |
| SPLEN2036932 | 0 | 0.000 | 3.497 |
| SPLEN2039379 | 0 | 0.000 | 10.362 |
| SPLEN2041585 | 0 | 0.000 | 40.674 |
| SPLEN2042714 | 0 | 0.000 | 40.674 |
| STOMA1000186 | 0 | 0.000 | 14.912 |
| STOMA1000189 | 0 | 0.000 | 1.865 |
| STOMA2000289 | 0 | 0.000 | 6.498 |
| STOMA2000395 | 0 | 0.000 | 5.092 |
| STOMA2000396 | 0 | 0.000 | 6.147 |
| STOMA2002052 | 0 | 0.000 | 6.634 |
| STOMA2004925 | 0 | 0.000 | 14.912 |
| SYNOV1000124 | 0 | 0.000 | 1.327 |
| SYNOV2001033 | 0 | 0.000 | 0.715 |
| SYNOV2001239 | 6.992 | 0 | 0.374 |
| SYNOV2001262 | 0 | 0.000 | 4.977 |
| SYNOV2017055 | 0 | 0.000 | 20.136 |
| SYNOV4000598 | 0 | 0.000 | 35.608 |
| SYNOV4001224 | 0 | 0.000 | 35.608 |
| SYNOV4001395 | 0 | 0.000 | 21.660 |
| SYNOV4009575 | 0 | 0.000 | 5.715 |
| T1ESE2000904 | 0 | 0.000 | 2.754 |
| TBAES2000059 | 0 | 0.000 | 1.797 |
| TBAES2003702 | 0 | 0.000 | 14.497 |
| TBAES2009387 | 0 | 0.000 | 0.638 |
| TCERX2000613 | 0 | 0.000 | 14.497 |
| TESOP2002273 | 0 | 0.000 | 4.841 |
| TESOP2006893 | 0 | 0.000 | 10.246 |
| TESTI1000023 | 0 | 0.000 | 2.000 |
| TESTI2000184 | 0 | 0.000 | 4.045 |
| TESTI2000356 | 0 | 0.000 | 20.914 |
| TESTI2000784 | 0 | 0.000 | 5.421 |
| TESTI2000970 | 0 | 0.000 | 5.798 |
| TESTI2001099 | 0 | 0.000 | 4.091 |
| TESTI2001153 | 0 | 0.000 | 5.699 |
| TESTI2001420 | 0 | 0.000 | 64.596 |
| TESTI2001829 | 0 | 0.000 | 1.013 |
| TESTI2001862 | 0 | 0.000 | 3.154 |
| TESTI2002149 | 0 | 0.000 | 3.990 |
| TESTI2002365 | 0 | 0.000 | 3.907 |
| TESTI2002632 | 0 | 0.000 | 12.913 |
| TESTI2002877 | 0 | 0.000 | 44.500 |
| TESTI2003005 | 0 | 0.000 | 3.623 |
| TESTI2003533 | 0 | 0.000 | 1.034 |
| TESTI2004031 | 8.934 | 0 | 0.478 |
| TESTI2004295 | 0 | 0.000 | 9.766 |
| TESTI2004737 | 0 | 0.000 | 2.113 |
| TESTI2004929 | 0 | 0.000 | 3.386 |
| TESTI2005731 | 0 | 0.000 | 3.941 |
| TESTI2005759 | 0 | 0.000 | 2.231 |
| TESTI2005908 | 0 | 0.000 | 1.878 |
| TESTI2006051 | 0 | 0.000 | 0.505 |
| TESTI2006111 | 0 | 0.000 | 24.590 |
| TESTI2006588 | 0 | 0.000 | 4.520 |
| TESTI2007211 | 0 | 0.000 | 3.939 |
| TESTI2007407 | 0 | 0.000 | 7.322 |
| TESTI2008240 | 0 | 0.000 | 2.982 |
| TESTI2008835 | 0 | 0.000 | 12.941 |
| TESTI2009520 | 0 | 0.000 | 0.894 |
| TESTI2009577 | 0 | 0.000 | 2.108 |
| TESTI2009785 | 0 | 0.000 | 6.845 |
| TESTI2010513 | 8.756 | 0 | 1.874 |
| TESTI2011683 | 0 | 0.000 | 38.615 |
| TESTI2014838 | 0 | 0.000 | 3.160 |
| TESTI2015213 | 0 | 0.000 | 0.612 |
| TESTI2015626 | 7.329 | 0 | 0.784 |
| TESTI2017954 | 0 | 0.000 | 0.790 |
| TESTI2018462 | 0 | 0.000 | 3.370 |
| TESTI2020871 | 0 | 0.000 | 64.596 |
| TESTI2021124 | 0 | 0.000 | 6.816 |
| TESTI2021358 | 0 | 0.000 | 40.357 |
| TESTI2021425 | 0 | 0.000 | 4.107 |
| TESTI2027165 | 0 | 0.000 | 64.596 |
| TESTI2029259 | 73.757 | 0 | 3.947 |
| TESTI2033441 | 0 | 0.000 | 2.618 |
| TESTI2034940 | 0 | 0.000 | 1.864 |
| TESTI2036913 | 0 | 0.000 | 24.204 |
| TESTI2036969 | 0 | 0.000 | 4.285 |
| TESTI2039177 | 0 | 0.000 | 4.943 |
| TESTI2044788 | 0 | 0.000 | 64.596 |
| TESTI2046456 | 0 | 0.000 | 1.104 |
| TESTI2047605 | 0 | 0.000 | 17.379 |
| TESTI2053723 | 0 | 0.000 | 5.399 |
| TESTI4000014 | 0 | 0.000 | 0.639 |
| TESTI4001467 | 0 | 0.000 | 6.603 |
| TESTI4002072 | 0 | 0.000 | 3.983 |
| TESTI4002195 | 0 | 0.000 | 15.959 |
| TESTI4002799 | 9.842 | 0 | 1.053 |
| TESTI4003703 | 0 | 0.000 | 5.277 |
| TESTI4003944 | 0 | 0.000 | 41.606 |
| TESTI4005399 | 0 | 0.000 | 9.300 |
| TESTI4005534 | 0 | 0.000 | 33.259 |
| TESTI4008797 | 0 | 0.000 | 14.824 |
| TESTI4009286 | 0 | 0.000 | 2.594 |
| TESTI4013441 | 0 | 14.788 | 0.374 |
| TESTI4013675 | 0 | 0.000 | 35.326 |
| TESTI4013894 | 0 | 0.000 | 33.408 |
| TESTI4014159 | 0 | 0.000 | 13.979 |
| TESTI4018886 | 0 | 0.000 | 64.596 |
| TESTI4024245 | 0 | 0.000 | 40.205 |
| TESTI4029671 | 0 | 0.000 | 22.183 |
| TESTI4037156 | 0 | 0.000 | 1.320 |
| TESTI4038284 | 41.043 | 0 | 0.000 |
| THYMU1000002 | 13.191 | 0 | 0.000 |
| THYMU1000016 | 0 | 0.000 | 2.304 |
| THYMU1000041 | 0 | 0.000 | 5.672 |
| THYMU1000083 | 0 | 0.000 | 10.729 |
| THYMU1000103 | 0 | 0.000 | 2.103 |
| THYMU1000109 | 0 | 0.000 | 0.939 |
| THYMU1000142 | 0 | 0.000 | 58.853 |
| THYMU1000316 | 0 | 0.000 | 58.853 |
| THYMU1000359 | 0 | 0.000 | 1.918 |
| THYMU1000374 | 0 | 0.000 | 3.573 |
| THYMU1000394 | 0 | 0.000 | 11.726 |
| THYMU1000428 | 0 | 0.000 | 0.678 |
| THYMU2000382 | 0 | 0.000 | 2.892 |
| THYMU2000436 | 20.534 | 0 | 1.099 |
| THYMU2000684 | 0 | 0.000 | 4.966 |
| THYMU2001007 | 0 | 0.000 | 6.471 |
| THYMU2001422 | 0 | 0.000 | 14.855 |
| THYMU2002583 | 0 | 0.000 | 0.618 |
| THYMU2002815 | 0 | 0.000 | 39.408 |
| THYMU2003012 | 0 | 0.000 | 2.090 |
| THYMU2003046 | 0 | 0.000 | 4.585 |
| THYMU2003419 | 0 | 0.000 | 3.594 |
| THYMU2003446 | 0 | 0.000 | 2.845 |
| THYMU2003891 | 0 | 0.000 | 0.174 |
| THYMU2003981 | 0 | 0.000 | 2.491 |
| THYMU2004152 | 0 | 0.000 | 1.184 |
| THYMU2004410 | 0 | 0.000 | 2.285 |
| THYMU2005546 | 12.190 | 0 | 0.000 |
| THYMU2006505 | 0 | 0.000 | 6.513 |
| THYMU2006813 | 0 | 0.000 | 1.887 |
| THYMU2007415 | 0 | 0.000 | 8.658 |
| THYMU2007467 | 0 | 0.000 | 4.818 |
| THYMU2007886 | 30.859 | 0 | 0.000 |
| THYMU2008686 | 0 | 0.000 | 14.535 |
| THYMU2008725 | 0 | 0.000 | 15.552 |
| THYMU2009658 | 0 | 0.000 | 0.418 |
| THYMU2010094 | 0 | 0.000 | 6.947 |
| THYMU2010448 | 0 | 0.000 | 6.315 |
| THYMU2012024 | 0 | 0.000 | 3.554 |
| THYMU2013916 | 0 | 0.000 | 2.646 |
| THYMU2015321 | 0 | 0.000 | 58.853 |
| THYMU2019021 | 0 | 0.000 | 5.796 |
| THYMU2020667 | 0 | 0.000 | 41.697 |
| THYMU2021684 | 0 | 0.000 | 58.853 |
| THYMU2024071 | 0 | 0.000 | 39.309 |
| THYMU2025319 | 0 | 0.000 | 39.408 |
| THYMU2031890 | 0 | 0.000 | 21.176 |
| THYMU2033070 | 0 | 0.000 | 58.853 |
| THYMU2034917 | 0 | 0.000 | 23.175 |
| THYMU2035735 | 0 | 0.000 | 11.014 |
| THYMU2036461 | 0 | 0.000 | 18.090 |
| THYMU3001472 | 0 | 0.000 | 20.097 |
| THYMU3002578 | 0 | 0.000 | 36.487 |
| THYMU3013386 | 0 | 0.000 | 1.786 |
| THYMU3021586 | 0 | 0.000 | 4.926 |
| THYMU3026350 | 0 | 0.000 | 17.045 |
| THYMU3028410 | 0 | 0.000 | 58.853 |
| THYMU3032798 | 0 | 0.000 | 2.306 |
| THYMU3034616 | 0 | 0.000 | 58.853 |
| THYMU3034671 | 0 | 0.000 | 19.911 |
| TOVAR2000476 | 50.572 | 0 | 0.000 |
| TRACH1000030 | 0 | 0.000 | 5.760 |
| TRACH1000038 | 0 | 0.000 | 8.984 |
| TRACH1000063 | 0 | 0.000 | 2.565 |
| TRACH1000181 | 0 | 0.000 | 5.650 |
| TRACH1000205 | 0 | 0.000 | 4.694 |
| TRACH2000079 | 0 | 0.000 | 0.646 |
| TRACH2000237 | 0 | 0.000 | 6.412 |
| TRACH2000248 | 0 | 0.000 | 2.496 |
| TRACH2000321 | 0 | 0.000 | 2.344 |
| TRACH2000359 | 0 | 0.000 | 3.557 |
| TRACH2000461 | 0 | 0.000 | 2.045 |
| TRACH2000472 | 0 | 0.000 | 9.166 |
| TRACH2000780 | 0 | 0.000 | 5.212 |
| TRACH2000959 | 0 | 0.000 | 0.582 |
| TRACH2001021 | 9.332 | 0 | 1.498 |
| TRACH2001192 | 0 | 0.000 | 40.205 |
| TRACH2001289 | 0 | 0.000 | 1.622 |
| TRACH2001432 | 0 | 0.000 | 1.327 |
| TRACH2001443 | 0 | 0.000 | 11.083 |
| TRACH2001463 | 0 | 0.000 | 3.652 |
| TRACH2001549 | 0 | 0.000 | 5.043 |
| TRACH2001596 | 0 | 0.000 | 24.409 |
| TRACH2001612 | 0 | 0.000 | 4.764 |
| TRACH2001810 | 0 | 0.000 | 0.926 |
| TRACH2002054 | 0 | 0.000 | 51.568 |
| TRACH2002537 | 0 | 0.000 | 2.212 |
| TRACH2003272 | 0 | 0.000 | 4.419 |
| TRACH2003323 | 0 | 0.000 | 2.123 |
| TRACH2005811 | 0 | 0.000 | 1.258 |
| TRACH2007399 | 0 | 0.000 | 1.685 |
| TRACH2008300 | 0 | 0.000 | 2.025 |
| TRACH2014124 | 0 | 0.000 | 3.693 |
| TRACH2014442 | 50.173 | 0 | 0.000 |
| TRACH2014997 | 0 | 0.000 | 25.316 |
| TRACH2015823 | 0 | 0.000 | 5.218 |
| TRACH2016481 | 0 | 0.000 | 51.568 |
| TRACH2017609 | 0 | 0.000 | 37.903 |
| TRACH2018950 | 0 | 0.000 | 17.915 |
| TRACH3000692 | 0 | 0.000 | 5.590 |
| TRACH3002192 | 0 | 0.000 | 4.463 |
| TRACH3003379 | 0 | 0.000 | 29.185 |
| TRACH3003547 | 0 | 0.000 | 5.044 |
| TRACH3003872 | 0 | 0.000 | 9.820 |
| TRACH3004068 | 0 | 0.000 | 1.700 |
| TRACH3004721 | 0 | 0.000 | 3.467 |
| TRACH3005699 | 0 | 0.000 | 22.893 |
| TRACH3006800 | 0 | 0.000 | 34.742 |
| TRACH3007479 | 0 | 0.000 | 3.848 |
| TRACH3008632 | 0 | 0.000 | 20.157 |
| TRACH3009008 | 0 | 0.000 | 5.843 |
| TRACH3009148 | 0 | 0.000 | 10.698 |
| TUTER1000014 | 100.000 | 0 | 0.000 |
| TUTER1000122 | 72.738 | 0 | 0.000 |
| TUTER1000137 | 95.706 | 0 | 0.000 |
| TUTER2000057 | 92.461 | 0 | 0.000 |
| TUTER2000283 | 100.000 | 0 | 0.000 |
| TUTER2000425 | 100.000 | 0 | 0.000 |
| TUTER2000904 | 62.217 | 0 | 3.330 |
| TUTER2000916 | 100.000 | 0 | 0.000 |
| TUTER2001286 | 100.000 | 0 | 0.000 |
| TUTER2001341 | 100.000 | 0 | 0.000 |
| TUTER2001387 | 100.000 | 0 | 0.000 |
| TUTER2001433 | 100.000 | 0 | 0.000 |
| TUTER2001461 | 100.000 | 0 | 0.000 |
| TUTER2002028 | 100.000 | 0 | 0.000 |
| TUTER2002074 | 100.000 | 0 | 0.000 |
| TUTER2002158 | 100.000 | 0 | 0.000 |
| TUTER2002228 | 100.000 | 0 | 0.000 |
| TUTER2002323 | 72.582 | 0 | 0.000 |
| TUTER2002356 | 100.000 | 0 | 0.000 |
| TUTER2002729 | 100.000 | 0 | 0.000 |
| UMVEN1000122 | 0 | 92.143 | 0.000 |
| UMVEN1000143 | 0 | 77.431 | 0.000 |
| UMVEN1000156 | 0 | 97.424 | 0.000 |
| UMVEN1000186 | 0 | 74.732 | 0.630 |
| UMVEN2000046 | 0 | 100.000 | 0.000 |
| UMVEN2000069 | 0 | 100.000 | 0.000 |
| UMVEN2000121 | 0 | 97.690 | 0.000 |
| UMVEN2000133 | 0 | 55.263 | 2.097 |
| UMVEN2000152 | 0 | 74.264 | 0.000 |
| UMVEN2000354 | 0 | 77.661 | 0.000 |
| UMVEN2000453 | 0 | 49.229 | 1.245 |
| UTERU100000$$ | 0 | 0.000 | 58.$$53 |
| UTERU1000015 | 0 | 0.000 | 100.000 |
| UTERU1000024 | 0 | 0.000 | 100.000 |
| UTERU1000031 | 0 | 0.000 | 100.000 |
| UTERU1000032 | 0 | 0.000 | 100.000 |
| UTERU1000057 | 0 | 0.000 | 100.000 |
| UTERU1000065 | 0 | 0.000 | 100.000 |
| UTERU1000077 | 0 | 0.000 | 100.000 |
| UTERU1000093 | 0 | 0.000 | 100.000 |
| UTERU1000096 | 0 | 0.000 | 24.268 |
| UTERU1000106 | 0 | 0.000 | 6.873 |
| UTERU1000109 | 0 | 0.000 | 100.000 |
| UTERU1000131 | 0 | 0.000 | 100.000 |
| UTERU1000138 | 0 | 0.000 | 100.000 |
| UTERU1000148 | 0 | 0.000 | 100.000 |
| UTERU1000160 | 0 | 0.000 | 100.000 |
| UTERU1000182 | 0 | 0.000 | 1.935 |
| UTERU1000183 | 0 | 0.000 | 100.000 |
| UTERU1000187 | 0 | 0.000 | 100.000 |
| UTERU1000192 | 0 | 0.000 | 100.000 |
| UTERU1000249 | 0 | 0.000 | 100.000 |
| UTERU1000337 | 0 | 0.000 | 100.000 |
| UTERU1000339 | 0 | 0.000 | 100.000 |
| UTERU1000384 | 0 | 0.000 | 8.500 |
| UTERU2000023 | 0 | 0.000 | 24.419 |
| UTERU2000047 | 0 | 0.000 | 100.000 |
| UTERU2000074 | 0 | 0.000 | 62.692 |
| UTERU2000095 | 0 | 0.000 | 0.987 |
| UTERU2000099 | 0 | 0.000 | 100.000 |
| UTERU2000154 | 0 | 0.000 | 100.000 |
| UTERU2000197 | 0 | 0.000 | 39.231 |
| UTERU2000218 | 0 | 0.000 | 100.000 |
| UTERU2000238 | 0 | 0.000 | 100.000 |
| UTERU2000243 | 0 | 0.000 | 100.000 |
| UTERU2000260 | 0 | 0.000 | 24.831 |
| UTERU2000263 | 0 | 0.000 | 2.444 |
| UTERU2000300 | 0 | 0.000 | 11.765 |
| UTERU2000329 | 0 | 0.000 | 68.264 |
| UTERU2000332 | 0 | 0.000 | 5.640 |
| UTERU2000338 | 0 | 0.000 | 3.979 |
| UTERU2000349 | 0 | 0.000 | 100.000 |
| UTERU2000377 | 0 | 0.000 | 100.000 |
| UTERU2000393 | 0 | 0.000 | 100.000 |
| UTERU2000418 | 0 | 0.000 | 34.205 |
| UTERU2000424 | 0 | 0.000 | 1.013 |
| UTERU2000465 | 0 | 0.000 | 100.000 |
| UTERU2000485 | 0 | 0.000 | 15.228 |
| UTERU2000517 | 0 | 0.000 | 100.000 |
| UTERU2000524 | 0 | 0.000 | 100.000 |
| UTERU2000537 | 0 | 0.000 | 12.239 |
| UTERU2000539 | 0 | 0.000 | 100.000 |
| UTERU2000541 | 0 | 0.000 | 7.522 |
| UTERU2000542 | 0 | 0.000 | 10.769 |
| UTERU2000546 | 0 | 0.000 | 100.000 |
| UTERU2000550 | 0 | 0.000 | 100.000 |
| UTERU2000569 | 0 | 0.000 | 100.000 |
| UTERU2000607 | 0 | 0.000 | 100.000 |
| UTERU2000629 | 0 | 0.000 | 100.000 |
| UTERU2000649 | 0 | 0.000 | 100.000 |
| UTERU2000663 | 0 | 0.000 | 100.000 |
| UTERU2000696 | 63.416 | 0 | 3.394 |
| UTERU2000794 | 0 | 0.000 | 100.000 |
| UTERU2000830 | 0 | 0.000 | 100.000 |
| UTERU2000844 | 0 | 0.000 | 100.000 |
| UTERU2000922 | 0 | 0.000 | 100.000 |
| UTERU2000925 | 0 | 0.000 | 12.420 |
| UTERU2001024 | 0 | 0.000 | 100.000 |
| UTERU2001110 | 0 | 0.000 | 24.268 |
| UTERU2001176 | 0 | 0.000 | 100.000 |
| UTERU2001281 | 0 | 0.000 | 100.000 |
| UTERU2001389 | 0 | 0.000 | 100.000 |
| UTERU2001409 | 0 | 0.000 | 100.000 |
| UTERU2001412 | 0 | 0.000 | 100.000 |
| UTERU2001504 | 0 | 0.000 | 100.000 |
| UTERU2001607 | 0 | 0.000 | 1.759 |
| UTERU2001658 | 0 | 0.000 | 10.564 |
| UTERU2001747 | 0 | 0.000 | 100.000 |
| UTERU2001876 | 0 | 0.000 | 100.000 |
| UTERU2002001 | 0 | 0.000 | 40.126 |
| UTERU2002011 | 0 | 0.000 | 100.000 |
| UTERU2002176 | 0 | 0.000 | 100.000 |
| UTERU2002198 | 0 | 0.000 | 64.596 |
| UTERU2002294 | 0 | 0.000 | 2.165 |
| UTERU2002332 | 0 | 0.000 | 100.000 |
| UTERU2002410 | 13.085 | 0 | 0.700 |
| UTERU2002473 | 0 | 0.000 | 100.000 |
| UTERU2002547 | 0 | 0.000 | 100.000 |
| UTERU2002662 | 0 | 0.000 | 4.244 |
| UTERU2002693 | 0 | 0.000 | 100.000 |
| UTERU2002733 | 0 | 0.000 | 100.000 |
| UTERU2002736 | 0 | 0.000 | 100.000 |
| UTERU2002737 | 0 | 0.000 | 100.000 |
| UTERU2002826 | 82.260 | 0 | 4.402 |
| UTERU2002841 | 0 | 0.000 | 64.596 |
| UTERU2002964 | 0 | 0.000 | 25.187 |
| UTERU2002993 | 0 | 0.000 | 64.596 |
| UTERU2003035 | 0 | 0.000 | 100.000 |
| UTERU2003057 | 0 | 0.000 | 3.085 |
| UTERU2003126 | 0 | 0.000 | 100.000 |
| UTERU2003135 | 0 | 0.000 | 100.000 |
| UTERU2003321 | 0 | 0.000 | 47.706 |
| UTERU2003399 | 0 | 0.000 | 100.000 |
| UTERU2003411 | 0 | 0.000 | 0.360 |
| UTERU2003456 | 0 | 0.000 | 12.899 |
| UTERU2003577 | 0 | 0.000 | 100.000 |
| UTERU2003704 | 0 | 0.000 | 20.529 |
| UTERU2003926 | 0 | 0.000 | 40.126 |
| UTERU2003973 | 0 | 0.000 | 100.000 |
| UTERU2004015 | 0 | 0.000 | 100.000 |
| UTERU2004037 | 0 | 0.000 | 5.230 |
| UTERU2004039 | 0 | 0.000 | 100.000 |
| UTERU2004061 | 0 | 0.000 | 100.000 |
| UTERU2004073 | 0 | 0.000 | 23.318 |
| UTERU2004163 | 0 | 0.000 | 100.000 |
| UTERU2004197 | 0 | 0.000 | 4.796 |
| UTERU2004299 | 0 | 0.000 | 100.000 |
| UTERU2004461 | 0 | 0.000 | 32.361 |
| UTERU2004520 | 0 | 0.000 | 39.037 |
| UTERU2004564 | 0 | 0.000 | 100.000 |
| UTERU2004664 | 0 | 0.000 | 4.048 |
| UTERU2004688 | 0 | 0.000 | 6.630 |
| UTERU2004698 | 0 | 0.000 | 100.000 |
| UTERU2004807 | 0 | 0.000 | 17.972 |
| UTERU2004861 | 0 | 0.000 | 56.434 |
| UTERU2004929 | 0 | 0.000 | 13.845 |
| UTERU2005004 | 0 | 0.000 | 41.697 |
| UTERU2005050 | 0 | 0.000 | 100.000 |
| UTERU2005069 | 0 | 0.000 | 64.596 |
| UTERU2005074 | 0 | 0.000 | 27.087 |
| UTERU2005179 | 0 | 0.000 | 100.000 |
| UTERU2005292 | 0 | 0.000 | 3.714 |
| UTERU2005346 | 0 | 0.000 | 100.000 |
| UTERU2005354 | 0 | 0.000 | 2.609 |
| UTERU2005446 | 0 | 0.000 | 24.816 |
| UTERU2005449 | 0 | 0.000 | 19.526 |
| UTERU2005450 | 0 | 0.000 | 100.000 |
| UTERU2005533 | 0 | 0.000 | 100.000 |
| UTERU2005593 | 0 | 0.000 | 4.016 |
| UTERU2005601 | 0 | 0.000 | 3.040 |
| UTERU2005621 | 0 | 0.000 | 5.377 |
| UTERU2005664 | 0 | 0.000 | 13.328 |
| UTERU2005822 | 0 | 0.000 | 100.000 |
| UTERU2005903 | 0 | 0.000 | 100.000 |
| UTERU2005905 | 0 | 0.000 | 100.000 |
| UTERU2006103 | 0 | 0.000 | 100.000 |
| UTERU2006115 | 0 | 0.000 | 9.347 |
| UTERU2006137 | 0 | 0.000 | 9.269 |
| UTERU2006182 | 0 | 0.000 | 100.000 |
| UTERU2006400 | 0 | 0.000 | 15.228 |
| UTERU2006412 | 0 | 0.000 | 100.000 |
| UTERU2006429 | 0 | 0.000 | 100.000 |
| UTERU2006486 | 0 | 0.000 | 51.819 |
| UTERU2006524 | 0 | 0.000 | 53.896 |
| UTERU2006547 | 0 | 0.000 | 24.268 |
| UTERU2006568 | 0 | 0.000 | 100.000 |
| UTERU2006593 | 0 | 0.000 | 3.522 |
| UTERU2006643 | 0 | 0.000 | 10.128 |
| UTERU2006651 | 0 | 0.000 | 100.000 |
| UTERU2006705 | 0 | 0.000 | 100.000 |
| UTERU2006899 | 0 | 0.000 | 100.000 |
| UTERU2007004 | 0 | 0.000 | 24.856 |
| UTERU2007075 | 0 | 0.000 | 100.000 |
| UTERU2007081 | 0 | 0.000 | 17.449 |
| UTERU2007128 | 0 | 0.000 | 100.000 |
| UTERU2007253 | 0 | 0.000 | 12.290 |
| UTERU2007267 | 0 | 0.000 | 100.000 |
| UTERU2007444 | 0 | 0.000 | 64.596 |
| UTERU2007499 | 0 | 0.000 | 100.000 |
| UTERU2007639 | 0 | 0.000 | 100.000 |
| UTERU2007724 | 0 | 0.000 | 15.672 |
| UTERU2007924 | 0 | 0.000 | 3.190 |
| UTERU2007942 | 0 | 0.000 | 100.000 |
| UTERU2008018 | 0 | 0.000 | 24.856 |
| UTERU2008019 | 0 | 0.000 | 100.000 |
| UTERU2008027 | 0 | 0.000 | 4.562 |
| UTERU2008040 | 0 | 0.000 | 14.037 |
| UTERU2008077 | 0 | 0.000 | 100.000 |
| UTERU2008085 | 0 | 0.000 | 0.323 |
| UTERU2008130 | 0 | 0.000 | 53.896 |
| UTERU2008302 | 0 | 0.000 | 3.187 |
| UTERU2008347 | 0 | 0.000 | 100.000 |
| UTERU2008426 | 0 | 0.000 | 26.260 |
| UTERU2008516 | 0 | 0.000 | 100.000 |
| UTERU2008561 | 0 | 0.000 | 100.000 |
| UTERU2008653 | 0 | 0.000 | 48.981 |
| UTERU2008705 | 0 | 0.000 | 100.000 |
| UTERU2008707 | 0 | 0.000 | 100.000 |
| UTERU2008747 | 0 | 0.000 | 10.541 |
| UTERU2008785 | 0 | 0.000 | 100.000 |
| UTERU2008845 | 0 | 0.000 | 100.000 |
| UTERU2008901 | 0 | 0.000 | 100.000 |
| UTERU2008930 | 0 | 0.000 | 100.000 |
| UTERU2008938 | 0 | 0.000 | 8.806 |
| UTERU2008939 | 0 | 0.000 | 17.716 |
| UTERU2008962 | 0 | 0.000 | 64.596 |
| UTERU2009094 | 0 | 0.000 | 100.000 |
| UTERU2009120 | 0 | 0.000 | 100.000 |
| UTERU2009131 | 0 | 0.000 | 100.000 |
| UTERU2009147 | 0 | 0.000 | 100.000 |
| UTERU2009206 | 0 | 0.000 | 100.000 |
| UTERU2009283 | 0 | 0.000 | 100.000 |
| UTERU2009335 | 0 | 0.000 | 10.852 |
| UTERU2009414 | 0 | 0.000 | 100.000 |
| UTERU2009435 | 0 | 0.000 | 12.756 |
| UTERU2009483 | 0 | 0.000 | 100.000 |
| UTERU2009510 | 0 | 0.000 | 17.926 |
| UTERU2009538 | 0 | 0.000 | 40.674 |
| UTERU2009540 | 0 | 0.000 | 100.000 |
| UTERU2009776 | 0 | 0.000 | 100.000 |
| UTERU2009904 | 0 | 0.000 | 100.000 |
| UTERU2009951 | 0 | 0.000 | 100.000 |
| UTERU2009972 | 0 | 0.000 | 16.901 |
| UTERU2010115 | 0 | 0.000 | 100.000 |
| UTERU2010124 | 0 | 0.000 | 100.000 |
| UTERU2010164 | 0 | 0.000 | 100.000 |
| UTERU2010226 | 0 | 0.000 | 9.408 |
| UTERU2010231 | 0 | 0.000 | 100.000 |
| UTERU2010304 | 0 | 0.000 | 100.000 |
| UTERU2010320 | 0 | 0.000 | 25.187 |
| UTERU2010417 | 7.026 | 0 | 1.128 |
| UTERU2010431 | 0 | 0.000 | 100.000 |
| UTERU2010525 | 0 | 0.000 | 14.053 |
| UTERU2010651 | 0 | 0.000 | 100.000 |
| UTERU2010724 | 0 | 0.000 | 100.000 |
| UTERU2010747 | 0 | 0.000 | 62.692 |
| UTERU2011195 | 0 | 0.000 | 100.000 |
| UTERU2011199 | 0 | 0.000 | 100.000 |
| UTERU2011220 | 0 | 0.000 | 4.669 |
| UTERU2011261 | 0 | 0.000 | 100.000 |
| UTERU2011287 | 0 | 0.000 | 39.146 |
| UTERU2011410 | 0 | 0.000 | 100.000 |
| UTERU2011574 | 0 | 0.000 | 100.000 |
| UTERU2011621 | 0 | 0.000 | 11.843 |
| UTERU2011657 | 0 | 0.000 | 100.000 |
| UTERU2011741 | 0 | 0.000 | 14.051 |
| UTERU2011806 | 0 | 0.000 | 49.807 |
| UTERU2011811 | 0 | 0.000 | 100.000 |
| UTERU2011897 | 0 | 0.000 | 100.000 |
| UTERU2011906 | 0 | 0.000 | 16.267 |
| UTERU2011962 | 0 | 0.000 | 100.000 |
| UTERU2011968 | 0 | 0.000 | 100.000 |
| UTERU2012031 | 0 | 0.000 | 53.896 |
| UTERU2012101 | 0 | 0.000 | 100.000 |
| UTERU2012114 | 0 | 0.000 | 100.000 |
| UTERU2012230 | 0 | 0.000 | 18.890 |
| UTERU2012252 | 0 | 0.000 | 25.609 |
| UTERU2012286 | 0 | 0.000 | 3.022 |
| UTERU2012333 | 0 | 0.000 | 100.000 |
| UTERU2012407 | 0 | 0.000 | 29.356 |
| UTERU2012526 | 0 | 0.000 | 11.013 |
| UTERU2012581 | 0 | 0.000 | 100.000 |
| UTERU2012610 | 0 | 0.000 | 100.000 |
| UTERU2012615 | 0 | 0.000 | 100.000 |
| UTERU2012688 | 0 | 0.000 | 51.568 |
| UTERU2012703 | 0 | 0.000 | 24.539 |
| UTERU2012715 | 0 | 0.000 | 34.205 |
| UTERU2012741 | 0 | 0.000 | 21.469 |
| UTERU2012767 | 0 | 0.000 | 6.351 |
| UTERU2012786 | 0 | 0.000 | 100.000 |
| UTERU2012856 | 0 | 0.000 | 8.416 |
| UTERU2012890 | 0 | 0.000 | 100.000 |
| UTERU2012938 | 0 | 0.000 | 48.981 |
| UTERU2012976 | 0 | 0.000 | 100.000 |
| UTERU2013048 | 0 | 0.000 | 100.000 |
| UTERU2013078 | 0 | 0.000 | 16.358 |
| UTERU2013231 | 0 | 0.000 | 100.000 |
| UTERU2013262 | 0 | 0.000 | 43.775 |
| UTERU2013280 | 0 | 0.000 | 100.000 |
| UTERU2013322 | 0 | 0.000 | 100.000 |
| UTERU2013483 | 0 | 0.000 | 100.000 |
| UTERU2013491 | 0 | 0.000 | 13.929 |
| UTERU2013502 | 0 | 0.000 | 40.214 |
| UTERU2013586 | 0 | 0.000 | 7.858 |
| UTERU2013926 | 0 | 0.000 | 100.000 |
| UTERU2013976 | 0 | 0.000 | 100.000 |
| UTERU2014001 | 0 | 0.000 | 100.000 |
| UTERU2014024 | 0 | 0.000 | 15.457 |
| UTERU2014167 | 0 | 0.000 | 1.087 |
| UTERU2014223 | 0 | 0.000 | 100.000 |
| UTERU2014398 | 0 | 0.000 | 100.000 |
| UTERU2014464 | 0 | 0.000 | 16.101 |
| UTERU2014548 | 0 | 0.000 | 39.231 |
| UTERU2014601 | 0 | 0.000 | 16.520 |
| UTERU2014631 | 0 | 0.000 | 100.000 |
| UTERU2014668 | 0 | 0.000 | 39.231 |
| UTERU2014678 | 0 | 0.000 | 48.981 |
| UTERU2014728 | 0 | 0.000 | 100.000 |
| UTERU2014898 | 0 | 0.000 | 1.815 |
| UTERU2014998 | 0 | 0.000 | 100.000 |
| UTERU2015062 | 0 | 0.000 | 17.070 |
| UTERU2015087 | 0 | 0.000 | 100.000 |
| UTERU2015108 | 0 | 0.000 | 7.526 |
| UTERU2015190 | 0 | 0.000 | 40.674 |
| UTERU2015198 | 0 | 0.000 | 100.000 |
| UTERU2015202 | 0 | 0.000 | 100.000 |
| UTERU2015405 | 0 | 0.000 | 100.000 |
| UTERU2015640 | 0 | 0.000 | 100.000 |
| UTERU2015653 | 0 | 0.000 | 4.175 |
| UTERU2015830 | 0 | 0.000 | 58.853 |
| UTERU2015880 | 0 | 0.000 | 100.000 |
| UTERU2016147 | 0 | 0.000 | 100.000 |
| UTERU2016157 | 0 | 0.000 | 100.000 |
| UTERU2016426 | 0 | 0.000 | 15.158 |
| UTERU2016464 | 0 | 0.000 | 100.000 |
| UTERU2016669 | 0 | 0.000 | 9.848 |
| UTERU2016757 | 0 | 0.000 | 54.394 |
| UTERU2016761 | 0 | 0.000 | 100.000 |
| UTERU2016799 | 0 | 0.000 | 10.404 |
| UTERU2016822 | 0 | 0.000 | 19.178 |
| UTERU2016896 | 0 | 0.000 | 100.000 |
| UTERU2016902 | 0 | 0.000 | 100.000 |
| UTERU2016979 | 0 | 0.000 | 12.336 |
| UTERU2016981 | 0 | 0.000 | 100.000 |
| UTERU2017123 | 0 | 0.000 | 100.000 |
| UTERU2017303 | 0 | 0.000 | 100.000 |
| UTERU2017421 | 0 | 0.000 | 100.000 |
| UTERU2017492 | 0 | 0.000 | 5.197 |
| UTERU2017613 | 0 | 0.000 | 12.264 |
| UTERU2017623 | 0 | 0.000 | 64.596 |
| UTERU2017632 | 0 | 0.000 | 64.596 |
| UTERU2017761 | 0 | 0.000 | 62.692 |
| UTERU2017762 | 0 | 0.000 | 39.037 |
| UTERU2017810 | 0 | 0.000 | 100.000 |
| UTERU2017988 | 0 | 0.000 | 100.000 |
| UTERU2018127 | 0 | 0.000 | 100.000 |
| UTERU2018180 | 0 | 0.000 | 100.000 |
| UTERU2018200 | 0 | 0.000 | 28.946 |
| UTERU2018333 | 0 | 0.000 | 100.000 |
| UTERU2018364 | 0 | 0.000 | 53.896 |
| UTERU2018514 | 0 | 0.000 | 100.000 |
| UTERU2018522 | 0 | 0.000 | 100.000 |
| UTERU2018523 | 0 | 0.000 | 40.126 |
| UTERU2018544 | 0 | 0.000 | 100.000 |
| UTERU2018566 | 0 | 0.000 | 21.258 |
| UTERU2018609 | 0 | 0.000 | 100.000 |
| UTERU2018674 | 0 | 0.000 | 100.000 |
| UTERU2018712 | 0 | 0.000 | 54.206 |
| UTERU2018784 | 0 | 0.000 | 100.000 |
| UTERU2018789 | 0 | 0.000 | 100.000 |
| UTERU2018811 | 0 | 0.000 | 100.000 |
| UTERU2018867 | 0 | 0.000 | 100.000 |
| UTERU2018881 | 0 | 0.000 | 100.000 |
| UTERU2018884 | 0 | 0.000 | 1.848 |
| UTERU2018955 | 0 | 0.000 | 100.000 |
| UTERU2019005 | 0 | 0.000 | 7.703 |
| UTERU2019038 | 0 | 0.000 | 100.000 |
| UTERU2019096 | 0 | 0.000 | 5.495 |
| UTERU2019163 | 0 | 0.000 | 100.000 |
| UTERU2019257 | 0 | 0.000 | 57.271 |
| UTERU2019453 | 0 | 0.000 | 100.000 |
| UTERU2019491 | 0 | 0.000 | 100.000 |
| UTERU2019534 | 0 | 0.000 | 100.000 |
| UTERU2019681 | 0 | 0.000 | 100.000 |
| UTERU2019706 | 0 | 0.000 | 54.394 |
| UTERU2019710 | 0 | 0.000 | 100.000 |
| UTERU2019940 | 0 | 0.000 | 51.819 |
| UTERU2019959 | 0 | 0.000 | 100.000 |
| UTERU2019964 | 0 | 0.000 | 100.000 |
| UTERU2020226 | 0 | 0.000 | 78.491 |
| UTERU2020242 | 0 | 0.000 | 100.000 |
| UTERU2020292 | 0 | 0.000 | 100.000 |
| UTERU2020351 | 0 | 0.000 | 100.000 |
| UTERU2020491 | 0 | 0.000 | 100.000 |
| UTERU2020718 | 0 | 0.000 | 100.000 |
| UTERU2021163 | 0 | 0.000 | 53.896 |
| UTERU2021380 | 0 | 0.000 | 100.000 |
| UTERU2021649 | 0 | 0.000 | 100.000 |
| UTERU2021820 | 0 | 0.000 | 29.732 |
| UTERU2022020 | 0 | 0.000 | 100.000 |
| UTERU2022773 | 0 | 0.000 | 100.000 |
| UTERU2022955 | 0 | 0.000 | 100.000 |
| UTERU2022981 | 0 | 0.000 | 100.000 |
| UTERU2023039 | 0 | 0.000 | 54.394 |
| UTERU2023045 | 0 | 0.000 | 10.794 |
| UTERU2023175 | 0 | 0.000 | 7.103 |
| UTERU2023262 | 0 | 0.000 | 15.042 |
| UTERU2023550 | 0 | 0.000 | 9.988 |
| UTERU2023651 | 0 | 0.000 | 29.963 |
| UTERU2023687 | 0 | 0.000 | 100.000 |
| UTERU2023712 | 0 | 0.000 | 100.000 |
| UTERU2023941 | 0 | 0.000 | 100.000 |
| UTERU2024002 | 0 | 0.000 | 100.000 |
| UTERU2024042 | 0 | 0.000 | 100.000 |
| UTERU2024141 | 0 | 0.000 | 100.000 |
| UTERU2024481 | 0 | 0.000 | 100.000 |
| UTERU2024656 | 0 | 0.000 | 51.568 |
| UTERU2024758 | 0 | 0.000 | 100.000 |
| UTERU2024820 | 0 | 0.000 | 100.000 |
| UTERU2024881 | 0 | 0.000 | 100.000 |
| UTERU2024969 | 0 | 0.000 | 11.490 |
| UTERU2025025 | 0 | 0.000 | 100.000 |
| UTERU2025041 | 0 | 0.000 | 100.000 |
| UTERU2025301 | 0 | 0.000 | 5.184 |
| UTERU2025366 | 0 | 0.000 | 100.000 |
| UTERU2025415 | 0 | 0.000 | 100.000 |
| UTERU2025579 | 0 | 0.000 | 100.000 |
| UTERU2025645 | 0 | 0.000 | 100.000 |
| UTERU2025891 | 0 | 0.000 | 100.000 |
| UTERU2026025 | 0 | 0.000 | 100.000 |
| UTERU2026090 | 0 | 0.000 | 100.000 |
| UTERU2026142 | 0 | 0.000 | 100.000 |
| UTERU2026203 | 0 | 0.000 | 54.394 |
| UTERU2026775 | 0 | 0.000 | 100.000 |
| UTERU2027023 | 0 | 0.000 | 100.000 |
| UTERU2027369 | 0 | 0.000 | 39.231 |
| UTERU2027591 | 0 | 0.000 | 100.000 |
| UTERU2027616 | 0 | 0.000 | 100.000 |
| UTERU2027941 | 0 | 0.000 | 100.000 |
| UTERU2028377 | 0 | 0.000 | 100.000 |
| UTERU2028734 | 0 | 0.000 | 26.183 |
| UTERU2029503 | 0 | 0.000 | 100.000 |
| UTERU2029660 | 0 | 0.000 | 100.000 |
| UTERU2029742 | 0 | 0.000 | 100.000 |
| UTERU2029953 | 0 | 0.000 | 100.000 |
| UTERU2030213 | 0 | 0.000 | 58.763 |
| UTERU2030270 | 0 | 0.000 | 100.000 |
| UTERU2030280 | 0 | 0.000 | 51.819 |
| UTERU2031060 | 0 | 0.000 | 100.000 |
| UTERU2031084 | 0 | 0.000 | 25.928 |
| UTERU2031268 | 0 | 0.000 | 100.000 |
| UTERU2031295 | 0 | 0.000 | 100.000 |
| UTERU2031521 | 0 | 0.000 | 100.000 |
| UTERU2031611 | 0 | 0.000 | 100.000 |
| UTERU2031703 | 0 | 0.000 | 100.000 |
| UTERU2031834 | 0 | 0.000 | 100.000 |
| UTERU2031851 | 0 | 0.000 | 100.000 |
| UTERU2032075 | 0 | 0.000 | 100.000 |
| UTERU2032220 | 0 | 0.000 | 100.000 |
| UTERU2032279 | 0 | 0.000 | 100.000 |
| UTERU2032726 | 0 | 0.000 | 100.000 |
| UTERU2033172 | 0 | 0.000 | 100.000 |
| UTERU2033375 | 0 | 0.000 | 1.385 |
| UTERU2033382 | 0 | 0.000 | 100.000 |
| UTERU2033420 | 0 | 0.000 | 100.000 |
| UTERU2033530 | 0 | 0.000 | 100.000 |
| UTERU2033577 | 0 | 0.000 | 100.000 |
| UTERU2034053 | 0 | 0.000 | 100.000 |
| UTERU2034147 | 0 | 0.000 | 58.853 |
| UTERU2034695 | 0 | 0.000 | 100.000 |
| UTERU2035114 | 0 | 0.000 | 51.568 |
| UTERU2035187 | 0 | 0.000 | 100.000 |
| UTERU2035231 | 0 | 0.000 | 100.000 |
| UTERU2035306 | 0 | 0.000 | 100.000 |
| UTERU2035323 | 0 | 0.000 | 100.000 |
| UTERU2035328 | 0 | 0.000 | 100.000 |
| UTERU2035331 | 0 | 0.000 | 100.000 |
| UTERU2035452 | 0 | 0.000 | 100.000 |
| UTERU2035469 | 0 | 0.000 | 100.000 |
| UTERU2035503 | 0 | 0.000 | 100.000 |
| UTERU2035745 | 0 | 0.000 | 100.000 |
| UTERU2035908 | 0 | 0.000 | 100.000 |
| UTERU2035926 | 0 | 0.000 | 100.000 |
| UTERU2035978 | 0 | 0.000 | 100.000 |
| UTERU2036089 | 0 | 0.000 | 100.000 |
| UTERU2036347 | 0 | 0.000 | 37.903 |
| UTERU2036507 | 0 | 0.000 | 100.000 |
| UTERU2036512 | 0 | 0.000 | 100.000 |
| UTERU2036530 | 0 | 0.000 | 100.000 |
| UTERU2036623 | 0 | 0.000 | 100.000 |
| UTERU2036690 | 0 | 0.000 | 100.000 |
| UTERU2037224 | 0 | 0.000 | 100.000 |
| UTERU2037361 | 0 | 0.000 | 100.000 |
| UTERU2037423 | 0 | 0.000 | 100.000 |
| UTERU2037577 | 0 | 0.000 | 100.000 |
| UTERU2037674 | 0 | 0.000 | 100.000 |
| UTERU2037791 | 0 | 0.000 | 100.000 |
| UTERU2037843 | 0 | 0.000 | 100.000 |
| UTERU2038171 | 0 | 0.000 | 100.000 |
| UTERU2038251 | 0 | 0.000 | 100.000 |
| UTERU3000226 | 0 | 0.000 | 62.692 |
| UTERU3000298 | 0 | 0.000 | 11.036 |
| UTERU3000402 | 0 | 0.000 | 100.000 |
| UTERU3000645 | 0 | 0.000 | 34.742 |
| UTERU3000665 | 0 | 0.000 | 100.000 |
| UTERU3000670 | 0 | 0.000 | 83.447 |
| UTERU3000727 | 0 | 0.000 | 26.926 |
| UTERU3000738 | 0 | 0.000 | 41.697 |
| UTERU3000828 | 0 | 0.000 | 100.000 |
| UTERU3000844 | 0 | 0.000 | 64.596 |
| UTERU3000899 | 0 | 0.000 | 33.654 |
| UTERU3000959 | 0 | 0.000 | 100.000 |
| UTERU3001029 | 0 | 0.000 | 100.000 |
| UTERU3001053 | 0 | 0.000 | 100.000 |
| UTERU3001059 | 0 | 0.000 | 100.000 |
| UTERU3001158 | 0 | 0.000 | 39.606 |
| UTERU3001240 | 0 | 0.000 | 100.000 |
| UTERU3001394 | 0 | 0.000 | 100.000 |
| UTERU3001542 | 0 | 0.000 | 58.853 |
| UTERU3001558 | 0 | 0.000 | 100.000 |
| UTERU3001571 | 0 | 0.000 | 51.819 |
| UTERU3001585 | 0 | 0.000 | 100.000 |
| UTERU3001632 | 0 | 0.000 | 100.000 |
| UTERU3001652 | 0 | 0.000 | 100.000 |
| UTERU3001766 | 0 | 0.000 | 54.206 |
| UTERU3001988 | 0 | 0.000 | 100.000 |
| UTERU3002209 | 0 | 0.000 | 100.000 |
| UTERU3002218 | 0 | 0.000 | 51.568 |
| UTERU3002383 | 0 | 0.000 | 100.000 |
| UTERU3002600 | 0 | 0.000 | 100.000 |
| UTERU3002620 | 0 | 0.000 | 100.000 |
| UTERU3002667 | 0 | 0.000 | 100.000 |
| UTERU3002701 | 0 | 0.000 | 100.000 |
| UTERU3002731 | 0 | 0.000 | 100.000 |
| UTERU3002768 | 0 | 0.000 | 100.000 |
| UTERU3002786 | 0 | 0.000 | 51.568 |
| UTERU3002993 | 0 | 0.000 | 100.000 |
| UTERU3003116 | 0 | 0.000 | 100.000 |
| UTERU3003135 | 0 | 0.000 | 39.146 |
| UTERU3003178 | 0 | 0.000 | 100.000 |
| UTERU3003465 | 0 | 0.000 | 100.000 |
| UTERU3003495 | 0 | 0.000 | 100.000 |
| UTERU3003523 | 0 | 0.000 | 100.000 |
| UTERU3003660 | 0 | 0.000 | 8.781 |
| UTERU3003774 | 0 | 0.000 | 100.000 |
| UTERU3003776 | 0 | 0.000 | 100.000 |
| UTERU3004384 | 0 | 0.000 | 53.896 |
| UTERU3004418 | 0 | 0.000 | 34.722 |
| UTERU3004477 | 0 | 0.000 | 57.271 |
| UTERU3004523 | 0 | 0.000 | 100.000 |
| UTERU3004616 | 0 | 0.000 | 100.000 |
| UTERU3004635 | 0 | 0.000 | 100.000 |
| UTERU3004709 | 0 | 0.000 | 51.819 |
| UTERU3004938 | 0 | 0.000 | 100.000 |
| UTERU3004992 | 0 | 0.000 | 100.000 |
| UTERU3005049 | 0 | 0.000 | 58.853 |
| UTERU3005159 | 0 | 0.000 | 41.606 |
| UTERU3005205 | 0 | 0.000 | 100.000 |
| UTERU3005230 | 0 | 0.000 | 58.249 |
| UTERU3005264 | 0 | 0.000 | 100.000 |
| UTERU3005422 | 0 | 0.000 | 25.298 |
| UTERU3005460 | 0 | 0.000 | 62.692 |
| UTERU3005536 | 0 | 0.000 | 62.692 |
| UTERU3005585 | 0 | 0.000 | 100.000 |
| UTERU3005767 | 26.525 | 0 | 7.098 |
| UTERU3005907 | 0 | 0.000 | 15.610 |
| UTERU3005970 | 0 | 0.000 | 62.692 |
| UTERU3006008 | 0 | 0.000 | 100.000 |
| UTERU3006228 | 0 | 0.000 | 100.000 |
| UTERU3006308 | 0 | 0.000 | 62.692 |
| UTERU3006538 | 0 | 0.000 | 100.000 |
| UTERU3006640 | 0 | 0.000 | 100.000 |
| UTERU3006687 | 0 | 0.000 | 100.000 |
| UTERU3006720 | 0 | 0.000 | 100.000 |
| UTERU3006798 | 0 | 0.000 | 100.000 |
| UTERU3006884 | 0 | 0.000 | 100.000 |
| UTERU3007021 | 0 | 0.000 | 34.742 |
| UTERU3007104 | 0 | 0.000 | 51.568 |
| UTERU3007108 | 0 | 0.000 | 100.000 |
| UTERU3007134 | 0 | 0.000 | 100.000 |
| UTERU3007419 | 0 | 0.000 | 62.692 |
| UTERU3007640 | 0 | 0.000 | 54.394 |
| UTERU3007731 | 0 | 0.000 | 100.000 |
| UTERU3007913 | 0 | 0.000 | 87.049 |
| UTERU3008280 | 0 | 0.000 | 37.121 |
| UTERU3008463 | 0 | 0.000 | 100.000 |
| UTERU3008660 | 0 | 0.000 | 100.000 |
| UTERU3008671 | 0 | 0.000 | 100.000 |
| UTERU3008722 | 0 | 0.000 | 100.000 |
| UTERU3008917 | 0 | 0.000 | 100.000 |
| UTERU3009259 | 0 | 0.000 | 53.896 |
| UTERU3009490 | 0 | 0.000 | 100.000 |
| UTERU3009517 | 0 | 0.000 | 54.206 |
| UTERU3009690 | 0 | 0.000 | 100.000 |
| UTERU3009775 | 0 | 0.000 | 100.000 |
| UTERU3009871 | 0 | 0.000 | 21.605 |
| UTERU3009979 | 0 | 0.000 | 100.000 |
| UTERU3009987 | 0 | 0.000 | 100.000 |
| UTERU3010029 | 0 | 0.000 | 100.000 |
| UTERU3010409 | 0 | 0.000 | 53.665 |
| UTERU3010526 | 0 | 0.000 | 100.000 |
| UTERU3010604 | 0 | 0.000 | 40.126 |
| UTERU3010892 | 0 | 0.000 | 100.000 |
| UTERU3010919 | 0 | 0.000 | 100.000 |
| UTERU3011063 | 0 | 0.000 | 100.000 |
| UTERU3011092 | 0 | 0.000 | 100.000 |
| UTERU3011273 | 0 | 0.000 | 100.000 |
| UTERU3011398 | 0 | 0.000 | 100.000 |
| UTERU3011558 | 0 | 0.000 | 100.000 |
| UTERU3011579 | 0 | 0.000 | 34.742 |
| UTERU3011795 | 0 | 0.000 | 100.000 |
| UTERU3011837 | 0 | 0.000 | 100.000 |
| UTERU3012293 | 0 | 0.000 | 100.000 |
| UTERU3012414 | 0 | 0.000 | 51.819 |
| UTERU3012476 | 0 | 0.000 | 100.000 |
| UTERU3012599 | 0 | 0.000 | 100.000 |
| UTERU3012999 | 0 | 0.000 | 100.000 |
| UTERU3013167 | 0 | 0.000 | 54.394 |
| UTERU3013302 | 0 | 0.000 | 58.853 |
| UTERU3013781 | 0 | 0.000 | 100.000 |
| UTERU3014274 | 0 | 0.000 | 100.000 |
| UTERU3014446 | 0 | 0.000 | 100.000 |
| UTERU3014611 | 0 | 0.000 | 100.000 |
| UTERU3014647 | 0 | 0.000 | 100.000 |
| UTERU3014791 | 0 | 0.000 | 100.000 |
| UTERU3014906 | 0 | 0.000 | 100.000 |
| UTERU3015011 | 0 | 0.000 | 16.834 |
| UTERU3015086 | 0 | 0.000 | 100.000 |
| UTERU3015299 | 0 | 0.000 | 100.000 |
| UTERU3015412 | 0 | 0.000 | 100.000 |
| UTERU3015500 | 0 | 0.000 | 100.000 |
| UTERU3015647 | 0 | 0.000 | 100.000 |
| UTERU3015844 | 0 | 0.000 | 100.000 |
| UTERU3016070 | 0 | 0.000 | 100.000 |
| UTERU3016273 | 0 | 0.000 | 53.896 |
| UTERU3016274 | 0 | 0.000 | 100.000 |
| UTERU3016308 | 0 | 0.000 | 100.000 |
| UTERU3016789 | 0 | 0.000 | 100.000 |
| UTERU3017176 | 0 | 0.000 | 100.000 |
| UTERU3017441 | 0 | 0.000 | 100.000 |
| UTERU3017626 | 0 | 0.000 | 100.000 |
| UTERU3017995 | 0 | 0.000 | 13.044 |
| UTERU3018154 | 0 | 0.000 | 100.000 |
| UTERU301$$172 | 0 | 0.000 | 3$$.038 |
| UTERU3018255 | 0 | 0.000 | 100.000 |
| UTERU3018616 | 0 | 0.000 | 25.167 |
| UTERU3018711 | 0 | 0.000 | 86.466 |
| UTERU3019078 | 0 | 0.000 | 100.000 |

Genes whose expression levels were negligibly low in each of the above-described libraries are shown with their clone names below.

3NB691000018, 3NB691000085, 3NB691000113; 3NB691000116, 3NB691000129, 3NB691000191, 3NB692000012, 3NB692000029, 3NB692000102, 3NB692000154, 3NB692000276, 3NB692000305, 3NB692000330, 3NB692000374, 3NB692000429, 3NB692000484, 3NB692000529, 3NB692000973, 3NB692001002, 3NB692001022, 3NB692001034, 3NB692001040, 3NB692001123, 3NB692001267, 3NB692001288, 3NB692001339, 3NB692001349, 3NB692001366, 3NB692001408, 3NB692001433, 3NB692001442, 3NB692001459, 3NB692001471, 3NB692001496, 3NB692001501, 3NB692001507, 3NB692001511, 3NB692001519, 3NB692001520, 3NB692001528, 3NB692001538, 3NB692001548, 3NB692001557, 3NB692001637, 3NB692001719, 3NB692001861, 3NB692002051, 3NB692002365, 3NB692002685, 3NB692002806,

3NB692003538, 3NB692004045, 3NB692004724, 3NB692005439, 3NB692006952, 3NB692008178, 3NB692008729, ACTVT2000380, ADIPS1000064, ADIPS2000069, ADIPS2000088, ADIPS2000245, ADIPS2000425, ADRGL1000002, ADRGL1000018, ADRGL1000033, ADRGL1000038, ADRGL1000065, ADRGL1000067, ADRGL1000111, ADRGL1000144, ADRGL1000147, ADRGL1000160, ADRGL1000165, ADRGL1000182, ADRGL2000056, ADRGL2000064, ADRGL2000074, ADRGL2000085, ADRGL2000097, ADRGL2000117, ADRGL2000142, ADRGL2000172, ADRGL2000248, ADRGL2000261, ADRGL2000323, ADRGL2000353, ADRGL2000384, ADRGL2000428, ADRGL2000636, ADRGL2000644, ADRGL2000968, ADRGL2001119, ADRGL2001229, ADRGL2001287, ADRGL2001301, ADRGL2001354, ADRGL2001554, ADRGL2001651, ADRGL2001756,

ADRGL2001830, ADRGL2001836, ADRGL2001854, ADRGL2002029, ADRGL2002191, ADRGL2002260, ADRGL2002392, ADRGL2002477, ADRGL2002679, ADRGL2002753, ADRGL2002857, ADRGL2003017, ADRGL2003329, ADRGL2003552, ADRGL2003585, ADRGL2003638, ADRGL2003684, ADRGL2003773, ADRGL2003785, ADRGL2004031, ADRGL2004077, ADRGL2004451, ADRGL2004459, ADRGL2004708, ADRGL2004724, ADRGL2004749, ADRGL2004770, ADRGL2004777, ADRGL2004833, ADRGL2005336, ADRGL2005564, ADRGL2005756, ADRGL2005838, ADRGL2005926, ADRGL2005961, ADRGL2006034, ADRGL2006124, ADRGL2006177, ADRGL2006193, ADRGL2006377, ADRGL2006617, ADRGL2006732, ADRGL2006767, ADRGL2006817, ADRGL2006846, ADRGL2006886, ADRGL2007080, ADRGL2007125, ADRGL2007283, ADRGL2007313,

ADRGL2007357, ADRGL2007636, ADRGL2007651, ADRGL2007810, ADRGL2007877, ADRGL2007906, ADRGL2007974, ADRGL2008023, ADRGL2008331, ADRGL2008337, ADRGL2008535, ADRGL2008966, ADRGL2009146, ADRGL2009324, ADRGL2009489, ADRGL2009533, ADRGL2009691, ADRGL2009755, ADRGL2010152, ADRGL2010315, ADRGL2010594, ADRGL2010860, ADRGL2010974, ADRGL2011190, ADRGL2011330, ADRGL2012038, ADRGL2012179, AHMSC1000138, ASTRO1000009, ASTRO1000018, ASTRO1000029, ASTRO1000096, ASTRO1000165, ASTRO2000011, ASTRO2000014, ASTRO2000095, ASTRO2000141, ASTRO2000191, ASTRO2000278, ASTRO2000372, ASTRO2000381, ASTRO2000403, ASTRO2000417, ASTRO2000482, ASTRO2000533, ASTRO2000653, ASTRO2000662, ASTRO2000725, ASTRO2001001, ASTRO2001008,

ASTRO2001029, ASTRO2001088, ASTRO2001107, ASTRO2001122, ASTRO2001227, ASTRO2001249, ASTRO2001480, ASTRO2001806, ASTRO2002024, ASTRO2002035, ASTRO2002064, ASTRO2002202, ASTRO2002459, ASTRO2002571, ASTRO2002632, ASTRO2002659, ASTRO2002693, ASTRO2002720, ASTRO2002733, ASTRO2002743, ASTRO2002842, ASTRO2003212, ASTRO2003461, ASTRO2003574, ASTRO2003581, ASTRO2003632, ASTRO2003740, ASTRO2003840, ASTRO2003960, ASTRO2004032, ASTRO2004051, ASTRO2004177, ASTRO2004217, ASTRO2004297, ASTRO2004584, ASTRO2004628, ASTRO2004751, ASTRO2004877, ASTRO2004974, ASTRO2005008, ASTRO2005081, ASTRO2005096, ASTRO2005242, ASTRO2005273, ASTRO2005343, ASTRO2005360, ASTRO2005413, ASTRO2005423, ASTRO2005553, ASTRO2005557,

ASTRO2005575, ASTRO2005593, ASTRO2005687, ASTRO2005726, ASTRO2005896, ASTRO2005902, ASTRO2006356, ASTRO2006475, ASTRO2006747, ASTRO2006920, ASTRO2006952, ASTRO2007221, ASTRO2007377, ASTRO2007515, ASTRO2007578, ASTRO2007643, ASTRO2007666, ASTRO2007956, ASTRO2007962, ASTRO2008040, ASTRO2008239, ASTRO2008409, ASTRO2008425, ASTRO2008777, ASTRO2008895, ASTRO2008960, ASTRO2008972, ASTRO2009068, ASTRO2009118, ASTRO2009177, ASTRO2009333, ASTRO2009458, ASTRO2009526, ASTRO2009879, ASTRO2010072, ASTRO2010615, ASTRO2010799, ASTRO2010962, ASTRO2010996, ASTRO2011149, ASTRO2011422, ASTRO2011426, ASTRO2011437, ASTRO2011461, ASTRO2011834, ASTRO2011893, ASTRO2012552, ASTRO2013124, ASTRO2013671, ASTRO2013802,

ASTRO2014135, ASTRO2014174, ASTRO2014211, ASTRO2014363, ASTRO2014392, ASTRO2014498, ASTRO2014561, ASTRO2014576, ASTRO2014863, ASTRO2014923, ASTRO2014961, ASTRO2015162, ASTRO2015185, ASTRO2015328, ASTRO2015529, ASTRO2015987, ASTRO2016044, ASTRO2016114, ASTRO2016148, ASTRO2016681, ASTRO2016819, ASTRO2016847, ASTRO2016892, ASTRO2017348, ASTRO2017627, ASTRO2018169, ASTRO2018373, ASTRO2018540, ASTRO2019039, ASTRO2019171, ASTRO3000154, ASTRO3000172, ASTRO3000177, ASTRO3000301, ASTRO3000474, ASTRO3000482, BEAST2000454, BGGI11000123, BGGI11000193, BGGI11000285, BGGI12000022, BGGI12000067, BGGI12000161, BGGI12000533, BGGI12000544, BGGI12000616, BGGI12000684, BGGI12000693, BGGI12000839, BGGI12001075,

BGGI12001097, BGGI12001241, BGGI12001247, BGGI12001682, BGGI12001714, BLADE1000176, BLADE2000181, BLADE2000256, BLADE2000340, BLADE2000389, BLADE2000463, BLADE2000492, BLADE2000579, BLADE2000640, BLADE2000866, BLADE2000938, BLADE2001031, BLADE2001133, BLADE2001138, BLADE2001141, BLADE2001371, BLADE2001458, BLADE2001575, BLADE2001753, BLADE2001935, BLADE2001940, BLADE2001987, BLADE2002073, BLADE2002310, BLADE2002730, BLADE2002738, BLADE2002744, BLADE2002782, BLADE2002947, BLADE2002972, BLADE2003474, BLADE2003916, BLADE2004089, BLADE2004345, BLADE2004389, BLADE2004462, BLADE2004628, BLADE2004670, BLADE2004849, BLADE2005036, BLADE2005038, BLADE2005229, BLADE2005459, BLADE2005792, BLADE2006043,

BLADE2006415, BLADE2006607, BLADE2006826, BLADE2006830, BLADE2006849, BLADE2006947, BLADE2007033, BLADE2007043, BLADE2007666, BLADE2007722, BLADE2007735, BLADE2007744, BLADE2007799, BLADE2007923, BLADE2007935, BLADE2007958, BLADE2008281, BLADE2008398, BLADE2008454, BLADE2008461, BLADE2008479, BLADE2008539, BLADE2008809, BLADE2008995, BLADE2009452, BLADE2009479, BLADE2009593, BNGH41000098, BNGH41000104, BNGH41000118, BNGH41000153, BNGH41000169, BNGH41000177, BNGH41000190, BNGH41000198, BNGH41000216, BNGH41000235, BNGH42000130, BNGH42000132, BNGH42000360, BNGH42000405, BNGH42000532, BNGH42000675, BNGH42000791, BNGH42000815, BNGH42000994, BNGH42001247, BNGH42001406, BNGH42001576, BNGH42001724,

BNGH42002168, BNGH42002244, BNGH42002387, BNGH42002489, BNGH42003529, BNGH42003570, BNGH42003641, BNGH420040.76, BNGH42004291, BNGH42004538, BNGH42004679, BNGH42005017, BNGH42005235, BNGH42005504, BNGH42005968, BNGH42006226, BNGH42006234, BNGH42007037, BNGH42007366, BNGH42007594, BNGH42007798, BNGH42008199, BNGH42008510, BNGH42008603, BNGH42008649, BNGH42008743, BNGH42008832, BNGH42008850, BNGH42008943, BNGH42009021, BNGH42009025, BRACE1000020, BRACE1000047, BRACE1000051, BRACE1000065, BRACE1000073, BRACE1000084, BRACE1000087, BRACE1000092, BRACE1000093, BRACE1000101, BRACE1000114, BRACE1000115, BRACE1000124, BRACE1000132, BRACE1000159, BRACE1000166, BRACE1000169, BRACE1000187, BRACE1000239,

BRACE1000258, BRACE1000451, BRACE1000475, BRACE1000572, BRACE2000061, BRACE2000077, BRACE2000078, BRACE2000100, BRACE2000109, BRACE2000141, BRACE2000148, BRACE2000183, BRACE2000229, BRACE2000232, BRACE2000245, BRACE2000256, BRACE2000266, BRACE2000267, BRACE2000280, BRACE2000300, BRACE2000307, BRACE2000331, BRACE2000332, BRACE2000351, BRACE2000368, BRACE2000392, BRACE2000441, BRACE2000487, BRACE2000489, BRACE2000505, BRACE2000525, BRACE2000526, BRACE2000531, BRACE2000545, BRACE2000553, BRACE2000565, BRACE2000577, BRACE2000640, BRACE2000650, BRACE2000677, BRACE2000698, BRACE2000718, BRACE2000733, BRACE2000743, BRACE2000753, BRACE2000815, BRACE2000864, BRACE2000885, BRACE2000893, BRACE2000905,

BRACE2000936, BRACE2000947, BRACE2000965, BRACE2000969, BRACE2000975, BRACE2000988, BRACE2001017, BRACE2001070, BRACE2001094, BRACE2001107, BRACE2001117, BRACE2001151, BRACE2001154, BRACE2001191, BRACE2001197, BRACE2001236, BRACE2001239, BRACE2001269, BRACE2001312, BRACE2001313, BRACE2001330, BRACE2001340, BRACE2001352, BRACE2001353, BRACE2001374, BRACE2001375, BRACE2001423, BRACE2001424, BRACE2001445, BRACE2001453, BRACE2001455, BRACE2001477, BRACE2001492, BRACE2001496, BRACE2001497, BRACE2001508, BRACE2001534, BRACE2001543, BRACE2001564, BRACE2001588, BRACE2001607, BRACE2001626, BRACE2001673, BRACE2001677, BRACE2001705, BRACE2001709, BRACE2001711, BRACE2001726, BRACE2001733, BRACE2001737,

BRACE2001742, BRACE2001777, BRACE2001779, BRACE2001834, BRACE2001859, BRACE2001865, BRACE2001872, BRACE2001881, BRACE2001892, BRACE2001898, BRACE2001923, BRACE2001942, BRACE2001944, BRACE2001954, BRACE2001972, BRACE2001973, BRACE2002026, BRACE2002034, BRACE2002050, BRACE2002081, BRACE2002091, BRACE2002139, BRACE2002142, BRACE2002151, BRACE2002157, BRACE2002202, BRACE2002206, BRACE2002227, BRACE2002260, BRACE2002297, BRACE2002304, BRACE2002356, BRACE2002392, BRACE2002394, BRACE2002431, BRACE2002441, BRACE2002444, BRACE2002458, BRACE2002467, BRACE2002468, BRACE2002495, BRACE2002582, BRACE2002585, BRACE2002589, BRACE2002590, BRACE2002606, BRACE2002613, BRACE2002614, BRACE2002617, BRACE2002623,

BRACE2002635, BRACE2002661, BRACE2002676, BRACE2002682, BRACE2002685, BRACE2002695, BRACE2002707, BRACE2002736, BRACE2002752, BRACE2002755, BRACE2002762, BRACE2002772, BRACE2002792, BRACE2002796, BRACE2002803, BRACE2002807, BRACE2002812, BRACE2002834, BRACE2002849, BRACE2002857, BRACE2002860, BRACE2002861, BRACE2002884, BRACE2002896, BRACE2002948, BRACE2003037, BRACE2003078, BRACE2003097, BRACE2003110, BRACE2003168, BRACE2003199, BRACE2003210, BRACE2003217, BRACE2003269, BRACE2003285, BRACE2003304, BRACE2003319, BRACE2003398, BRACE2003420, BRACE2003428, BRACE2003431, BRACE2003539, BRACE2003540, BRACE2003594, BRACE2003598, BRACE2003609, BRACE2003614, BRACE2003628, BRACE2003639, BRACE2003700,

BRACE2003713, BRACE2003766, BRACE2003788, BRACE2003800, BRACE2003802, BRACE2003825, BRACE2003827, BRACE2003845, BRACE2003847, BRACE2003848, BRACE2003873, BRACE2003885, BRACE2003887, BRACE2003905, BRACE2003928, BRACE2003944, BRACE2003954, BRACE2003982, BRACE2003987, BRACE2005034, BRACE2005047, BRACE2005063, BRACE2005083, BRACE2005090, BRACE2005099, BRACE2005118, BRACE2005124, BRACE2005138, BRACE2005151, BRACE2005160, BRACE2005169, BRACE2005189, BRACE2005193, BRACE2005198, BRACE2005216, BRACE2005243, BRACE2005251, BRACE2005253, BRACE2005298, BRACE2005328, BRACE2005329, BRACE2005337, BRACE2005348, BRACE2005363, BRACE2005395, BRACE2005408, BRACE2005433, BRACE2005434, BRACE2005448, BRACE2005450,

BRACE2005453, BRACE2005457, BRACE2005460, BRACE2005489, BRACE2005518, BRACE2005556, BRACE2005562, BRACE2005624, BRACE2005642, BRACE2005661, BRACE2005667, BRACE2005681, BRACE2005731, BRACE2005742, BRACE2005762, BRACE2005773, BRACE2005787, BRACE2005795, BRACE2005800, BRACE2005858, BRACE2005869, BRACE2005875, BRACE2005881, BRACE2005898, BRACE2005904, BRACE2005906, BRACE2005907, BRACE2005911, BRACE2005937, BRACE2005949, BRACE2005981, BRACE2005991, BRACE2006055, BRACE2006072, BRACE2006075, BRACE2006089, BRACE2006102, BRACE2006105, BRACE2006122, BRACE2006137, BRACE2006143, BRACE2006162, BRACE2006167, BRACE2006174, BRACE2006240, BRACE2006245, BRACE2006249, BRACE2006258, BRACE2006264, BRACE2006274,

BRACE2006281, BRACE2006298, BRACE2006319, BRACE2006344, BRACE2006363, BRACE2006378, BRACE2006380, BRACE2006393, BRACE2006413, BRACE2006417, BRACE2006453, BRACE2006459, BRACE2006488, BRACE2006514, BRACE2006534, BRACE2006540, BRACE2006547, BRACE2006564, BRACE2006587, BRACE2006598, BRACE2006685, BRACE2006703, BRACE2006743, BRACE2006753, BRACE2006783, BRACE2006821, BRACE2006833, BRACE2006859, BRACE2006871, BRACE2006909, BRACE2006911, BRACE2006924, BRACE2006935, BRACE2006937, BRACE2006951, BRACE2006982, BRACE2006986, BRACE2007013, BRACE2007068, BRACE2007087, BRACE2007089, BRACE2007109, BRACE2007138, BRACE2007191, BRACE2007197, BRACE2007201, BRACE2007203, BRACE2007232, BRACE2007254, BRACE2007262,

BRACE2007274, BRACE2007281, BRACE2007295, BRACE2007358, BRACE2007396, BRACE2007398, BRACE2007401, BRACE2007411, BRACE2007447, BRACE2007477, BRACE2007502, BRACE2007503, BRACE2007522, BRACE2007527, BRACE2007538, BRACE2007563, BRACE2007567, BRACE2007577, BRACE2007621, BRACE2007640, BRACE2007641, BRACE2007646, BRACE2007663, BRACE2007671, BRACE2007682, BRACE2007685, BRACE2007708, BRACE2007727, BRACE2007749, BRACE2007760, BRACE2007761, BRACE2007764, BRACE2007767, BRACE2007768, BRACE2007769, BRACE2007784, BRACE2007786, BRACE2007799, BRACE2007801, BRACE2007832, BRACE2007836, BRACE2007868, BRACE2007882, BRACE2007888, BRACE2007902, BRACE2007920, BRACE2007937, BRACE2007944, BRACE2007952, BRACE2008008,

BRACE2008015, BRACE2008021, BRACE2008097, BRACE2008102, BRACE2008114, BRACE2008144, BRACE2008172, BRACE2008216, BRACE2008218, BRACE2008251, BRACE2008276, BRACE2008295, BRACE2008308, BRACE2008336, BRACE2008358, BRACE2008361, BRACE2008380, BRACE2008385, BRACE2008399, BRACE2008401, BRACE2008402, BRACE2008410, BRACE2008420, BRACE2008443, BRACE2008473, BRACE2008480, BRACE2008481, BRACE2008488, BRACE2008495, BRACE2008500, BRACE2008553, BRACE2008594, BRACE2008604, BRACE2008615, BRACE2008622, BRACE2008629, BRACE2008655, BRACE2008656, BRACE2008706, BRACE2008708, BRACE2008710, BRACE2008754, BRACE2008797, BRACE2008847, BRACE2008857, BRACE2008861, BRACE2008867, BRACE2008881, BRACE2008883, BRACE2008917,

BRACE2008919, BRACE2008948, BRACE2008960, BRACE2008968, BRACE2008999, BRACE2009014, BRACE2009016, BRACE2009031, BRACE2009044, BRACE2009045, BRACE2009053, BRACE2009122, BRACE2009131, BRACE2009185, BRACE2009188, BRACE2009212, BRACE2009221, BRACE2009235, BRACE2009238, BRACE2009255, BRACE2009270, BRACE2009272, BRACE2009274, BRACE2009275, BRACE2009291, BRACE2009293, BRACE2009307, BRACE2009311, BRACE2009318, BRACE2009323, BRACE2009366, BRACE2009421, BRACE2009428, BRACE2009434, BRACE2009437, BRACE2009483, BRACE2009533, BRACE2009558, BRACE2009566, BRACE2009591, BRACE2009620, BRACE2009654, BRACE2009721, BRACE2009732, BRACE2009754, BRACE2009828, BRACE2009886, BRACE2009907, BRACE2009957, BRACE2010170,

BRACE2010171, BRACE2010203, BRACE2010208, BRACE2010336, BRACE2010348, BRACE2010440, BRACE2010444, BRACE2010477, BRACE2010489, BRACE2010535, BRACE2010550, BRACE2010598, BRACE2010632, BRACE2010642, BRACE2010651, BRACE2010669, BRACE2010684, BRACE2010753, BRACE2010813, BRACE2010828, BRACE2010837, BRACE2010888, BRACE2010937, BRACE2010983, BRACE2011007, BRACE2011025, BRACE2011199, BRACE2011265, BRACE2011389, BRACE2011478, BRACE2011545, BRACE2011592, BRACE2011611, BRACE2011646, BRACE2011677, BRACE2011703, BRACE2011747, BRACE2011838, BRACE2011892, BRACE2011904, BRACE2011947, BRACE2012061, BRACE2012062, BRACE2012185, BRACE2012222, BRACE2012232, BRACE2012291, BRACE2012317, BRACE2012510, BRACE2012625,

BRACE2012653, BRACE2012814, BRACE2012833, BRACE2012838, BRACE2012936, BRACE2012947, BRACE2013009, BRACE2013010, BRACE2013126, BRACE2013132, BRACE2013149, BRACE2013369, BRACE2013806, BRACE2013855, BRACE2013911, BRACE2014108, BRACE2014161, BRACE2014232, BRACE2014257, BRACE2014268, BRACE2014306, BRACE2014475, BRACE2014657, BRACE2014746, BRACE2014780, BRACE2014818, BRACE2014821, BRACE2014824, BRACE2014850, BRACE2014854, BRACE2015031, BRACE2015058, BRACE2015132, BRACE2015262, BRACE2015270, BRACE2015287, BRACE2015314, BRACE2015352, BRACE2015368, BRACE2015412, BRACE2015436, BRACE2015741, BRACE2015756, BRACE2015969, BRACE2015995, BRACE2016058, BRACE2016194, BRACE2016315, BRACE2016325, BRACE2016335,

BRACE2016362, BRACE2016366, BRACE2016465, BRACE2016516, BRACE2016583, BRACE2016708, BRACE2016821, BRACE2016896, BRACE2016981, BRACE2017108, BRACE2017124, BRACE2017298, BRACE2017359, BRACE2017410, BRACE2017438, BRACE2017580, BRACE2017587, BRACE2017720, BRACE2017844, BRACE2017872, BRACE2017929, BRACE2017934, BRACE2017985, BRACE2017992, BRACE2018337, BRACE2018443, BRACE2018448, BRACE2018568, BRACE2018700, BRACE2018727, BRACE2018736, BRACE2018759, BRACE2018762, BRACE2018782, BRACE2018847, BRACE2019004, BRACE2019044, BRACE2019244, BRACE2019258, BRACE2019327, BRACE2019348, BRACE2019371, BRACE2019467, BRACE2019510, BRACE2019519, BRACE2019528, BRACE2019618, BRACE2019696, BRACE2019703, BRACE2019843,

BRACE2019865, BRACE2019872, BRACE2019877, BRACE2020028, BRACE2020077, BRACE2020097, BRACE2020157, BRACE2020278, BRACE2020358, BRACE2020467, BRACE2020652, BRACE2020709, BRACE2020742, BRACE2020756, BRACE2020863, BRACE2020982, BRACE2021014, BRACE2021143, BRACE2021148, BRACE2021245, BRACE2021254, BRACE2021264, BRACE2021344, BRACE2021433, BRACE2021478, BRACE2021541, BRACE2021670, BRACE2021695, BRACE2021708, BRACE2021721, BRACE2021932, BRACE2021936, BRACE2022030, BRACE2022158, BRACE2022196, BRACE2022270, BRACE2022328, BRACE2022389, BRACE2022394, BRACE2022435, BRACE2022448, BRACE2022450, BRACE2022549, BRACE2022633, BRACE2022693, BRACE2022848, BRACE2022928, BRACE2022990, BRACE2023069, BRACE2023070,

BRACE2023223, BRACE2023268, BRACE2023284, BRACE2023404, BRACE2023451, BRACE2023540, BRACE2023633, BRACE2023718, BRACE2023727, BRACE2023744, BRACE2023800, BRACE2023801, BRACE2024074, BRACE2024222, BRACE2024610, BRACE2024627, BRACE2024781, BRACE2024826, BRACE2025018, BRACE2025316, BRACE2025333, BRACE2025437, BRACE2025452, BRACE2025492, BRACE2025710, BRACE2025796, BRACE2025992, BRACE2026054, BRACE2026131, BRACE2026141, BRACE2026142, BRACE2026293, BRACE2026294, BRACE2026404, BRACE2026675, BRACE2026725, BRACE2026836, BRACE2026920, BRACE2026971, BRACE2027018, BRACE2027221, BRACE2027258, BRACE2027312, BRACE2027382, BRACE2027389, BRACE2027408, BRACE2027643, BRACE2027767, BRACE2027770, BRACE2027879,

BRACE2027970, BRACE2028171, BRACE2028392, BRACE2028410, BRACE2028636, BRACE2028741, BRACE2028956, BRACE2028970, BRACE2029112, BRACE2029356, BRACE2029396, BRACE2029581, BRACE2029644, BRACE2029849, BRACE2030039, BRACE2030096, BRACE2030323, BRACE2030326, BRACE2030345, BRACE2030736, BRACE2030883, BRACE2030884, BRACE2031154, BRACE2031232, BRACE2031527, BRACE2031531, BRACE2031553, BRACE2031899, BRACE2032044, BRACE2032090, BRACE2032134, BRACE2032148, BRACE2032182, BRACE2032329, BRACE2032385, BRACE2032436, BRACE2032477, BRACE2032502, BRACE2032538, BRACE2032584, BRACE2032823, BRACE2033128, BRACE2033394, BRACE2033478, BRACE2033720, BRACE2033786, BRACE2034434, BRACE2034523, BRACE2034616, BRACE2034622,

BRACE2034870, BRACE2035003, BRACE2035120, BRACE2035124, BRACE2035191, BRACE2035278, BRACE2035381, BRACE2035441, BRACE2035485, BRACE2036005, BRACE2036096, BRACE2036743, BRACE2036830, BRACE2036834, BRACE2036900, BRACE2037132, BRACE2037294, BRACE2037295, BRACE2037299, BRACE2037431, BRACE2037692, BRACE2037847, BRACE2038114, BRACE2038269, BRACE2038271, BRACE2038329, BRACE2038492, BRACE2038551, BRACE2038618, BRACE2039009, BRACE2039128, BRACE2039170, BRACE2039249, BRACE2039286, BRACE2039297, BRACE2039327, BRACE2039362, BRACE2039475, BRACE2039600, BRACE2039607, BRACE2039624, BRACE2039734, BRACE2039823, BRACE2039832, BRACE2039893, BRACE2039986, BRACE2040138, BRACE2040325, BRACE2040514, BRACE2040640,

BRACE2040987, BRACE2041009, BRACE2041095, BRACE2041200, BRACE2041264, BRACE2041890, BRACE2041898, BRACE2042394, BRACE2042398, BRACE2042550, BRACE2042620, BRACE2042628, BRACE2042873, BRACE2043036, BRACE2043105, BRACE2043142, BRACE2043248, BRACE2043349, BRACE2043381, BRACE2043774, BRACE2043942, BRACE2044057, BRACE2044105, BRACE2044263, BRACE2044286, BRACE2044304, BRACE2044473, BRACE2044510, BRACE2044640, BRACE2044816, BRACE2044946, BRACE2044949, BRACE2045300, BRACE2045428, BRACE2045596, BRACE2045772, BRACE2045947, BRACE2045954, BRACE2046251, BRACE2046295, BRACE2046976, BRACE2047011, BRACE2047232, BRACE2047350, BRACE2047377, BRACE2047385, BRACE2047492, BRACE2047558, BRACE2047573, BRACE2047835,

BRACE2047893, BRACE2047975, BRACE3000071, BRACE3000404, BRACE3000520, BRACE3000697, BRACE3000840, BRACE3000973, BRACE3001002, BRACE3001058, BRACE3001066, BRACE3001113, BRACE3001217, BRACE3001299, BRACE3001384, BRACE3001391, BRACE3001403, BRACE3001595, BRACE3001754, BRACE3001948, BRACE3001973, BRACE3002184, BRACE3002298, BRACE3002344, BRACE3002390, BRACE3002420, BRACE3002508, BRACE3002541, BRACE3002756, BRACE3003004, BRACE3003026, BRACE3003053, BRACE3003192, BRACE3003413, BRACE3003595, BRACE3003698, BRACE3003866, BRACE3003920, BRACE3004046, BRACE3004058, BRACE3004113, BRACE3004150, BRACE3004205, BRACE3004358, BRACE3004435, BRACE3004477, BRACE3004767, BRACE3004772, BRACE3004783, BRACE3004843,

BRACE3004862, BRACE3004880, BRACE3004887, BRACE3004981, BRACE3004996, BRACE3004998, BRACE3005103, BRACE3005127, BRACE3005145, BRACE3005217, BRACE3005225, BRACE3005430, BRACE3005499, BRACE3005760, BRACE3005870, BRACE3005903, BRACE3005989, BRACE3006113, BRACE3006185, BRACE3006226, BRACE3006462, BRACE3006463, BRACE3006553, BRACE3006872, BRACE3006917, BRACE3007058, BRACE3007084, BRACE3007085, BRACE3007322, BRACE3007472, BRACE3007480, BRACE3007559, BRACE3007625, BRACE3007642, BRACE3007649, BRACE3007767, BRACE3007869, BRACE3008036, BRACE3008066, BRACE3008092, BRACE3008095, BRACE3008137, BRACE3008255, BRACE3008337, BRACE3008384, BRACE3008720, BRACE3009044, BRACE3009075, BRACE3009090, BRACE3009127,

BRACE3009197, BRACE3009237, BRACE3009265, BRACE3009297, BRACE3009377, BRACE3009392, BRACE3009416, BRACE3009539, BRACE3009574, BRACE3009701, BRACE3009708, BRACE3009724, BRACE3009993, BRACE3010076, BRACE3010079, BRACE3010086, BRACE3010221, BRACE3010283, BRACE3010315, BRACE3010397, BRACE3010416, BRACE3010435, BRACE3010438, BRACE3010702, BRACE3010862, BRACE3011271, BRACE3011421, BRACE3011447, BRACE3011505, BRACE3011634, BRACE3011774, BRACE3012357, BRACE3012574, BRACE3012806, BRACE3012930, BRACE3013119, BRACE3013418, BRACE3013576, BRACE3013740, BRACE3013780, BRACE3013874, BRACE3013936, BRACE3013986, BRACE3014005, BRACE3014068, BRACE3014231, BRACE3014290, BRACE3014317, BRACE3014523, BRACE3014714,

BRACE3014807, BRACE3014942, BRACE3015090, BRACE3015121, BRACE3015262, BRACE3015521, BRACE3015808, BRACE3015829, BRACE3015894, BRACE3015898, BRACE3016020, BRACE3016122, BRACE3016167, BRACE3016170, BRACE3016580, BRACE3016788, BRACE3016810, BRACE3016862, BRACE3016884, BRACE3017083, BRACE3017253, BRACE3018083, BRACE3018308, BRACE3018963, BRACE3019055, BRACE3019071, BRACE3019084, BRACE3019570, BRACE3019611, BRACE3019817, BRACE3019828, BRACE3019941, BRACE3020151, BRACE3020194, BRACE3020286, BRACE3020356, BRACE3020594, BRACE3020669, BRACE3020671, BRACE3020890, BRACE3021148, BRACE3021430, BRACE3021517, BRACE3021805, BRACE3022051, BRACE3022303, BRACE3022312, BRACE3022340, BRACE3022847, BRACE3023604,

BRACE3023912, BRACE3024073, BRACE3024359, BRACE3024379, BRACE3024444, BRACE3024497, BRACE3024537, BRACE3024659, BRACE3024662, BRACE3024879, BRACE3025153, BRACE3025302, BRACE3025457, BRACE3025531, BRACE3025627, BRACE3025630, BRACE3025719, BRACE3026008, BRACE3026075, BRACE3026161, BRACE3026290, BRACE3026345, BRACE3026456, BRACE3026735, BRACE3026802, BRACE3026844, BRACE3026947, BRACE3026993, BRACE3027242, BRACE3027256, BRACE3027326, BRACE3027480, BRACE3027931, BRACE3027976, BRACE3028214, BRACE3028241, BRACE3028360, BRACE3028895, BRACE3028998, BRACE3029005, BRACE3029021, BRACE3029205, BRACE3029447, BRACE3030057, BRACE3030103, BRACE3030538, BRACE3030748, BRACE3030866, BRACE3031161, BRACE3031184,

BRACE3031185, BRACE3031315, BRACE3031372, BRACE3031579, BRACE3031728, BRACE3031838, BRACE3031843, BRACE3032427, BRACE3032455, BRACE3032537, BRACE3032538, BRACE3032631, BRACE3032771, BRACE3032980, BRACE3032983, BRACE3033525, BRACE3034159, BRACE3034183, BRACE3034389, BRACE3034964, BRACE3034993, BRACE3035168, BRACE3035227, BRACE3036156, BRACE3036271, BRACE3037612, BRACE3037637, BRACE3037803, BRACE3038012, BRACE3038030, BRACE3038569, BRACE3038570, BRACE3038687, BRACE3038760, BRACE3039288, BRACE3039358, BRACE3039378, BRACE3039454, BRACE3040012, BRACE3040239, BRACE3040504, BRACE3040644, BRACE3040856, BRACE3041059, BRACE3041162, BRACE3041827, BRACE3042046, BRACE3042210, BRACE3042326, BRACE3042409,

BRACE3042432, BRACE3042594, BRACE3043597, BRACE3044090, BRACE3044172, BRACE3044247, BRACE3044377, BRACE3044389, BRACE3044495, BRACE3045011, BRACE3045033, BRACE3045078, BRACE3045145, BRACE3045424, BRACE3045708, BRACE3045981, BRACE3046049, BRACE3046152, BRACE3046294, BRACE3046450, BRACE3046466, BRACE3046491, BRACE3046609, BRACE3046762, BRACE3046837, BRACE3046855, BRACE3046966, BRACE3047018, BRACE3047482, BRACE3047801, BRALZ1000047, BRALZ1000103, BRALZ2000189, BRALZ2000263, BRALZ2000988, BRALZ2001038, BRALZ2001279, BRALZ2001350, BRALZ2001445, BRALZ2001559, BRALZ2001743, BRALZ2001834, BRALZ2002174, BRALZ2002418, BRALZ2002477, BRALZ2003119, BRALZ2003330, BRALZ2003453, BRALZ2003525, BRALZ2004397,

BRALZ2005467, BRALZ2005471, BRALZ2005888, BRALZ2005950, BRALZ2005992, BRALZ2006288, BRALZ2006474, BRALZ2006560, BRALZ2006734, BRALZ2006800, BRALZ2006976, BRALZ2007376, BRALZ2007545, BRALZ2007576, BRALZ2007661, BRALZ2007790, BRALZ2007793, BRALZ2007952, BRALZ2008031, BRALZ2008166, BRALZ2008462, BRALZ2008617, BRALZ2008696, BRALZ2008763, BRALZ2008869, BRALZ2008930, BRALZ2009486, BRALZ2010186, BRALZ2010842, BRALZ2011337, BRALZ2011796, BRALZ2011880, BRALZ2012050, BRALZ2012183, BRALZ2012848, BRALZ2013621, BRALZ2013690, BRALZ2014054, BRALZ2014316, BRALZ2014484, BRALZ2014951, BRALZ2015168, BRALZ2015811, BRALZ2016085, BRALZ2016498, BRALZ2017531, BRALZ2017607, BRALZ2017620, BRALZ2017844, BRALZ2018342,

BRALZ2018492, BRALZ2018834, BRALZ2019047, BRAMY1000095, BRAMY1000098, BRAMY1000130, BRAMY1000157, BRAMY1000173, BRAMY2000021, BRAMY2000025, BRAMY2000052, BRAMY2000076, BRAMY2000086, BRAMY2000151, BRAMY2000181, BRAMY2000196, BRAMY2000231, BRAMY2000277, BRAMY2000354, BRAMY2000388, BRAMY2000411, BRAMY2000508, BRAMY2000562, BRAMY2000653, BRAMY2000814, BRAMY2000893, BRAMY2000946, BRAMY2000981, BRAMY2000987, BRAMY2001068, BRAMY2001114, BRAMY2001203, BRAMY2001282, BRAMY2001367, BRAMY2001427, BRAMY2001473, BRAMY2001678, BRAMY2002005, BRAMY2002044, BRAMY2002072, BRAMY2002158, BRAMY2002311, BRAMY2002339, BRAMY2002364, BRAMY2002369, BRAMY2002479, BRAMY2002493, BRAMY2002525, BRAMY2002584, BRAMY2002604,

BRAMY2002621, BRAMY2002638, BRAMY2002739, BRAMY2002799, BRAMY2002853, BRAMY2002862, BRAMY2002956, BRAMY2003008, BRAMY2003037, BRAMY2003117, BRAMY2003287, BRAMY2003325, BRAMY2003529, BRAMY2003538, BRAMY2003585, BRAMY2003634, BRAMY2003653, BRAMY2003681, BRAMY2003893, BRAMY2003897, BRAMY2003898, BRAMY2003904, BRAMY2003926, BRAMY2003929, BRAMY2004031, BRAMY2004058, BRAMY2004183, BRAMY2004335, BRAMY2004351, BRAMY2004352, BRAMY2004387, BRAMY2004492, BRAMY2004524, BRAMY2004537, BRAMY2004542, BRAMY2004756, BRAMY2004771, BRAMY2005052, BRAMY2005064, BRAMY2005094, BRAMY2005151, BRAMY2005182, BRAMY2005244, BRAMY2005391, BRAMY2005488, BRAMY2005576, BRAMY2005648, BRAMY2005662, BRAMY2005708, BRAMY2005711,

BRAMY2006092, BRAMY2006118, BRAMY2006149, BRAMY2006375, BRAMY2006397, BRAMY2006411, BRAMY2006459, BRAMY2006466, BRAMY2006659, BRAMY2006779, BRAMY2006809, BRAMY2006879, BRAMY2007043, BRAMY2007078, BRAMY2007110, BRAMY2007185, BRAMY2007244, BRAMY2007249, BRAMY2007255, BRAMY2007287, BRAMY2007308, BRAMY2007411, BRAMY2007458, BRAMY2007486, BRAMY2007546, BRAMY2007567, BRAMY2007594, BRAMY2007610, BRAMY2007613, BRAMY2007653, BRAMY2007753, BRAMY2007859, BRAMY2008333, BRAMY2008382, BRAMY2008563, BRAMY2008583, BRAMY2008977, BRAMY2008979, BRAMY2009013, BRAMY2009023, BRAMY2009034, BRAMY2009105, BRAMY2009192, BRAMY2009344, BRAMY2009349, BRAMY2009394, BRAMY2009489, BRAMY2009508, BRAMY2009528, BRAMY2009557,

BRAMY2009612, BRAMY2009693, BRAMY2010068, BRAMY2010135, BRAMY2010145, BRAMY2010171, BRAMY2010208, BRAMY2010239, BRAMY2010272, BRAMY2010290, BRAMY2010357, BRAMY2010366, BRAMY2010395, BRAMY2010441, BRAMY2010442, BRAMY2010464, BRAMY2010581, BRAMY2010798, BRAMY2010808, BRAMY2010919, BRAMY2011064, BRAMY2011178, BRAMY2011196, BRAMY2011216, BRAMY2011253, BRAMY2011446, BRAMY2011480, BRAMY2011639, BRAMY2011679, BRAMY2011811, BRAMY2011841, BRAMY2011846, BRAMY2011849, BRAMY2011872, BRAMY2011961, BRAMY2012017, BRAMY2012091, BRAMY2012119, BRAMY2012162, BRAMY2012163, BRAMY2012277, BRAMY2012340, BRAMY2012426, BRAMY2012497, BRAMY2012517, BRAMY2012555, BRAMY2012568, BRAMY2012581, BRAMY2012691, BRAMY2012721,

BRAMY2012731, BRAMY2012776, BRAMY2012880, BRAMY2012881, BRAMY2013169, BRAMY2013200, BRAMY2013216, BRAMY2013405, BRAMY2013414, BRAMY2013572, BRAMY2013590, BRAMY2013621, BRAMY2013722, BRAMY2013736, BRAMY2013756, BRAMY2013944, BRAMY2013975, BRAMY2014019, BRAMY2014205, BRAMY2014387, BRAMY2014462, BRAMY2014469, BRAMY2014541, BRAMY2014754, BRAMY2014813, BRAMY2015211, BRAMY2015251, BRAMY2015311, BRAMY2015420, BRAMY2015503, BRAMY2015516, BRAMY2015550, BRAMY2015551, BRAMY2015559, BRAMY2015688, BRAMY2015705, BRAMY2015782, BRAMY2015833, BRAMY2015855, BRAMY2015890, BRAMY2015911, BRAMY2015925, BRAMY2016002, BRAMY2016009, BRAMY2016020, BRAMY2016070, BRAMY2016251, BRAMY2016325, BRAMY2016327, BRAMY2016386,

BRAMY2016706, BRAMY2016771, BRAMY2016892, BRAMY2016938, BRAMY2016953, BRAMY2017014, BRAMY2017249, BRAMY2017348, BRAMY2017450, BRAMY2017455, BRAMY2017528, BRAMY2017864, BRAMY2018027, BRAMY2018106, BRAMY2018122, BRAMY2018126, BRAMY2018272, BRAMY2018273, BRAMY2018279, BRAMY2018295, BRAMY2018308, BRAMY2018396, BRAMY2018467, BRAMY2018635, BRAMY2018785, BRAMY2019055, BRAMY2019111, BRAMY2019277, BRAMY2019300, BRAMY2019468, BRAMY2019509, BRAMY2019554, BRAMY2019600, BRAMY2019643, BRAMY2019710, BRAMY2019963, BRAMY2019985, BRAMY2020050, BRAMY2020058, BRAMY2020106, BRAMY2020270, BRAMY2020354, BRAMY2020355, BRAMY2020412, BRAMY2020445, BRAMY2020466, BRAMY2020520, BRAMY2020574, BRAMY2020634, BRAMY2020713,

BRAMY2021040, BRAMY2021063, BRAMY2021098, BRAMY2021139, BRAMY2021142, BRAMY2021276, BRAMY2021310, BRAMY2021388, BRAMY2021498, BRAMY2021523, BRAMY2021710, BRAMY2021732, BRAMY2021746, BRAMY2021825, BRAMY2021867, BRAMY2021962, BRAMY2021982, BRAMY2022052, BRAMY2022160, BRAMY2022263, BRAMY2022301, BRAMY2022320, BRAMY2022383, BRAMY2022445, BRAMY2022454, BRAMY2022493, BRAMY2022525, BRAMY2022532, BRAMY2022723, BRAMY2022786, BRAMY2022796, BRAMY2022929, BRAMY2022980, BRAMY2022984, BRAMY2023060, BRAMY2023110, BRAMY2023115, BRAMY2023161, BRAMY2023172, BRAMY2023238, BRAMY2023327, BRAMY2023358, BRAMY2023406, BRAMY2023482, BRAMY2023514, BRAMY2023719, BRAMY2023729, BRAMY2023786, BRAMY2023852, BRAMY2023863,

BRAMY2023939, BRAMY2024004, BRAMY2024073, BRAMY2024312, BRAMY2024374, BRAMY2024390, BRAMY2024416, BRAMY2024449, BRAMY2024489, BRAMY2024497, BRAMY2024514, BRAMY2024530, BRAMY2024535, BRAMY2024545, BRAMY2024576, BRAMY2024593, BRAMY2024711, BRAMY2024715, BRAMY2024728, BRAMY2024827, BRAMY2024849, BRAMY2025024, BRAMY2025032, BRAMY2025121, BRAMY2025175, BRAMY2025218, BRAMY2025230, BRAMY2025272, BRAMY2025277, BRAMY2025495, BRAMY2025812, BRAMY2025996, BRAMY2026009, BRAMY2026091, BRAMY2026168, BRAMY2026300, BRAMY2026347, BRAMY2026395, BRAMY2026405, BRAMY2026533, BRAMY2026538, BRAMY2026713, BRAMY2026778, BRAMY2026824, BRAMY2026899, BRAMY2026902, BRAMY2026904, BRAMY2026976, BRAMY2027073, BRAMY2027114,

BRAMY2027140, BRAMY2027242, BRAMY2027396, BRAMY2027451, BRAMY2027461, BRAMY2027467, BRAMY2027587, BRAMY2027714, BRAMY2027717, BRAMY2027739, BRAMY2027752, BRAMY2027782, BRAMY2028072, BRAMY2028183, BRAMY2028188, BRAMY2028269, BRAMY2028282, BRAMY2028472 BRAMY2028491, BRAMY2028516, BRAMY2028565, BRAMY2028593, BRAMY2028682, BRAMY2028783, BRAMY2028796, BRAMY2028856, BRAMY2028914, BRAMY2029204, BRAMY2029602, BRAMY2029988, BRAMY2030098, BRAMY2030109, BRAMY2030206, BRAMY2030477, BRAMY2030545, BRAMY2030607, BRAMY2030702, BRAMY2030703, BRAMY2030768, BRAMY2030799, BRAMY2030859, BRAMY2030915, BRAMY2031317, BRAMY2031377, BRAMY2031442, BRAMY2032014, BRAMY2032231, BRAMY2032242, BRAMY2032290, BRAMY2032311,

BRAMY2032317, BRAMY2032344, BRAMY2032589, BRAMY2032678, BRAMY2032696, BRAMY2032777, BRAMY2032994, BRAMY2033003, BRAMY2033116, BRAMY2033240, BRAMY2033267, BRAMY2033316, BRAMY2033332, BRAMY2033506, BRAMY2033594, BRAMY2033895, BRAMY2033914, BRAMY2034072, BRAMY2034185, BRAMY2034187, BRAMY2034305, BRAMY2034561, BRAMY2034920, BRAMY2034993, BRAMY2035070, BRAMY2035449, BRAMY2035545, BRAMY2035606, BRAMY2035718, BRAMY2035719, BRAMY2035801, BRAMY2035869, BRAMY2035923, BRAMY2036076, BRAMY2036079, BRAMY2036254, BRAMY2036266, BRAMY2036387, BRAMY2036396, BRAMY2036459, BRAMY2036567, BRAMY2036615, BRAMY2036699, BRAMY2036913, BRAMY2036918, BRAMY2036968, BRAMY2037147, BRAMY2037328, BRAMY2037609, BRAMY2037629,

BRAMY2037800, BRAMY2037823, BRAMY2037971, BRAMY2038100, BRAMY2038128, BRAMY2038317, BRAMY2038475, BRAMY2038484, BRAMY2038516, BRAMY2038832, BRAMY2038846, BRAMY2038887, BRAMY2038904, BRAMY2039287, BRAMY2039300, BRAMY2039314, BRAMY2039341, BRAMY2039630, BRAMY2039695, BRAMY2039728, BRAMY2039872, BRAMY2040051, BRAMY2040159, BRAMY2040214, BRAMY2040478, BRAMY2040592, BRAMY2040915, BRAMY2041261, BRAMY2041347, BRAMY2041378, BRAMY2041384, BRAMY2041434, BRAMY2041507, BRAMY2041542, BRAMY2041738, BRAMY2042122, BRAMY2042131, BRAMY2042178, BRAMY2042195, BRAMY2042244, BRAMY2042549, BRAMY2042596, BRAMY2042612, BRAMY2042641, BRAMY2042711, BRAMY2042760, BRAMY2042804, BRAMY2042899, BRAMY2042918, BRAMY2043069,

BRAMY2043103, BRAMY2043314, BRAMY2043345, BRAMY2044246, BRAMY2044317, BRAMY2044349, BRAMY2044686, BRAMY2044838, BRAMY2045036, BRAMY2045299, BRAMY2045471, BRAMY2045530, BRAMY2046109, BRAMY2046478, BRAMY2046489, BRAMY2046537, BRAMY2046667, BRAMY2046742, BRAMY2046871, BRAMY2046984, BRAMY2046989, BRAMY2047169, BRAMY2047280, BRAMY2047420, BRAMY2047676, BRAMY2047692, BRAMY2047746, BRAMY2047751, BRAMY2047765, BRAMY2047884, BRAMY2047948, BRAMY3000206, BRAMY3000210, BRAMY3000213, BRAMY3000224, BRAMY3000254, BRAMY3000307, BRAMY3000692, BRAMY3001356, BRAMY3001401, BRAMY3001409, BRAMY3001794, BRAMY3002120, BRAMY3002312, BRAMY3002329, BRAMY3002508, BRAMY3002620, BRAMY3002803, BRAMY3002805, BRAMY3002886,

BRAMY3002991, BRAMY3003026, BRAMY3003109, BRAMY3003205, BRAMY3003310, BRAMY3003566, BRAMY3003723, BRAMY3004126, BRAMY3004224, BRAMY3004364, BRAMY3004377, BRAMY3004672, BRAMY3004800, BRAMY3004900, BRAMY3004919, BRAMY3005091, BRAMY3005656, BRAMY3005912, BRAMY3005932, BRAMY3006032, BRAMY3006297, BRAMY3006761, BRAMY3007078, BRAMY3007206, BRAMY3007311, BRAMY3007350, BRAMY3007449, BRAMY3007471, BRAMY3007609, BRAMY3007723, BRAMY3007968, BRAMY3008044, BRAMY3008088, BRAMY3008096, BRAMY3008335, BRAMY3008466, BRAMY3008505, BRAMY3008650, BRAMY3008937, BRAMY3009158, BRAMY3009207, BRAMY3009491, BRAMY3009556, BRAMY3009782, BRAMY3009811, BRAMY3009904, BRAMY3010008, BRAMY3010264, BRAMY3010321, BRAMY3010411,

BRAMY3010492, BRAMY3010603, BRAMY3010654, BRAMY4000089, BRAMY4000095, BRAMY4000229, BRAMY4000277, BRAMY4000915, BRAMY4000962, BRAMY4001173, BRAMY4001200, BRAMY4001234, BRAMY4001449, BRAMY4001652, BRAMY4001863, BRAMY4001913, BRAMY4002575, BRAMY4002628, BRASW1000053, BRASW1000125, BRAWH1000001, BRAWH1000002, BRAWH1000007, BRAWH1000037, BRAWH1000045, BRAWH1000083, BRAWH1000094, BRAWH1000116, BRAWH1000127, BRAWH1000130, BRAWH1000157, BRAWH1000162, BRAWH1000164, BRAWH1000168, BRAWH1000174, BRAWH1000180, BRAWH1000369, BRAWH2000034, BRAWH2000048, BRAWH2000071, BRAWH2000093, BRAWH2000109, BRAWH2000131, BRAWH2000142, BRAWH2000169, BRAWH2000177, BRAWH2000218, BRAWH2000232, BRAWH2000248, BRAWH2000256,

BRAWH2000274, BRAWH2000323, BRAWH2000353, BRAWH2000370, BRAWH2000409, BRAWH2000417, BRAWH2000443, BRAWH2000460, BRAWH2000471, BRAWH2000476, BRAWH2000482, BRAWH2000494, BRAWH2000500, BRAWH2000503, BRAWH2000522, BRAWH2000554, BRAWH2000595, BRAWH2000636, BRAWH2000643, BRAWH2000651, BRAWH2000682, BRAWH2000686, BRAWH2000752, BRAWH2000859, BRAWH2000866, BRAWH2000892, BRAWH2000901, BRAWH2000924, BRAWH2000926, BRAWH2000944, BRAWH2000984, BRAWH2001019, BRAWH2001037, BRAWH2001052, BRAWH2001103, BRAWH2001129, BRAWH2001137, BRAWH2001159, BRAWH2001166, BRAWH2001171, BRAWH2001186, BRAWH2001203, BRAWH2001238, BRAWH2001239, BRAWH2001255, BRAWH2001321, BRAWH2001355, BRAWH2001378, BRAWH2001395, BRAWH2001406,

BRAWH2001418, BRAWH2001424, BRAWH2001436, BRAWH2001438, BRAWH2001459, BRAWH2001461, BRAWH2001484, BRAWH2001492, BRAWH2001503, BRAWH2001519, BRAWH2001530, BRAWH2001535, BRAWH2001589, BRAWH2001662, BRAWH2001666, BRAWH2001671, BRAWH2001679, BRAWH2001686, BRAWH2001701, BRAWH2001842, BRAWH2001862, BRAWH2001873, BRAWH2001940, BRAWH2001973, BRAWH2002047, BRAWH2002060, BRAWH2002062, BRAWH2002125, BRAWH2002191, BRAWH2002318, BRAWH2002333, BRAWH2002352, BRAWH2002383, BRAWH2002549, BRAWH2002560, BRAWH2002623, BRAWH2002630, BRAWH2002635, BRAWH2002662, BRAWH2002679, BRAWH2002725, BRAWH2002761, BRAWH2002811, BRAWH2002861, BRAWH2002911, BRAWH2002963, BRAWH2002967, BRAWH2002976, BRAWH2003000, BRAWH2003025,

BRAWH2003135, BRAWH2003240, BRAWH2003307, BRAWH2003355, BRAWH2003366, BRAWH2003662, BRAWH2003689, BRAWH2003818, BRAWH2003832, BRAWH2003948, BRAWH2003964, BRAWH2004044, BRAWH2004068, BRAWH2004095, BRAWH2004136, BRAWH2004217, BRAWH2004580, BRAWH2004731, BRAWH2004779, BRAWH2004842, BRAWH2004884, BRAWH2004923, BRAWH2005008, BRAWH2005068, BRAWH2005074, BRAWH2005225, BRAWH2005315, BRAWH2005517, BRAWH2005524, BRAWH2005533, BRAWH2005812, BRAWH2005974, BRAWH2005998, BRAWH2006044, BRAWH2006083, BRAWH2006207, BRAWH2006301, BRAWH2006395, BRAWH2006405, BRAWH2006450, BRAWH2006493, BRAWH2006526, BRAWH2006543, BRAWH2006622, BRAWH2006760, BRAWH2006960, BRAWH2006989, BRAWH2006996, BRAWH2007069, BRAWH2007308,

BRAWH2007406, BRAWH2007550, BRAWH2007570, BRAWH2007591, BRAWH2007658, BRAWH2007664, BRAWH2007800, BRAWH2007808, BRAWH2007825, BRAWH2007862, BRAWH2007890, BRAWH2008128, BRAWH2008219, BRAWH2008255, BRAWH2008292, BRAWH2008297, BRAWH2008366, BRAWH2008380, BRAWH2008540, BRAWH2008577, BRAWH2008706, BRAWH2008790, BRAWH2008903, BRAWH2008956, BRAWH2008993, BRAWH2009112, BRAWH2009227, BRAWH2009261, BRAWH2009263, BRAWH2009298, BRAWH2009304, BRAWH2009307, BRAWH2009360, BRAWH2009393, BRAWH2009485, BRAWH2009490, BRAWH2009558, BRAWH2009590, BRAWH2009626, BRAWH2009678, BRAWH2009695, BRAWH2009907, BRAWH2010000, BRAWH2010069, BRAWH2010084, BRAWH2010136, BRAWH2010318, BRAWH2010329, BRAWH2010354, BRAWH2010364,

BRAWH2010536, BRAWH2010552, BRAWH2010584, BRAWH2010618, BRAWH2010619, BRAWH2010754, BRAWH2010980, BRAWH2011066, BRAWH2011079, BRAWH2011096, BRAWH2011155, BRAWH2011166, BRAWH2011204, BRAWH2011286, BRAWH2011294, BRAWH2011400, BRAWH2011762, BRAWH2011795, BRAWH2011796, BRAWH2011812, BRAWH2011823, BRAWH2011860, BRAWH2011920, BRAWH2011958, BRAWH2012054, BRAWH2012077, BRAWH2012161, BRAWH2012162, BRAWH2012164, BRAWH2012258, BRAWH2012277, BRAWH2012326, BRAWH2012538, BRAWH2012540, BRAWH2012619, BRAWH2012749, BRAWH2012827, BRAWH2012866, BRAWH2012963, BRAWH2013047, BRAWH2013219, BRAWH2013294, BRAWH2013748, BRAWH2013866, BRAWH2013871, BRAWH2013914, BRAWH2013941, BRAWH2013955, BRAWH2014053, BRAWH2014234,

BRAWH2014379, BRAWH2014414, BRAWH2014511, BRAWH2014662, BRAWH2014717, BRAWH2014729, BRAWH2014876, BRAWH2014934, BRAWH2014954, BRAWH2015247, BRAWH2015349, BRAWH2015375, BRAWH2015385, BRAWH2015595, BRAWH2015728, BRAWH2015853, BRAWH2015866, BRAWH2016028, BRAWH2016106, BRAWH2016160, BRAWH2016209, BRAWH2016221, BRAWH2016223, BRAWH2016269, BRAWH2016305, BRAWH2016439, BRAWH2016446, BRAWH2016514, BRAWH2016562, BRAWH2016655, BRAWH2016679, BRAWH2016702, BRAWH2016724, BRAWH2016785, BRAWH2016792, BRAWH2016800, BRAWH2017002, BRAWH2017103, BRAWH2017250, BRAWH2017294, BRAWH2017305, BRAWH2017379, BRAWH2017407, BRAWH2017433, BRAWH2017523, BRAWH2017534, BRAWH2017542, BRAWH2017635, BRAWH2017913, BRAWH2018206,

BRAWH2018267, BRAWH2018282, BRAWH2018317, BRAWH2018506, BRAWH2018514, BRAWH2018527, BRAWH2018601, BRAWH2018729, BRAWH2018875, BRAWH2019053, BRAWH2019055, BRAWH2019198, BRAWH3000078, BRAWH3000088, BRAWH3000100, BRAWH3000166, BRAWH3000329, BRAWH3000410, BRAWH3000446, BRAWH3000491, BRAWH3000604, BRAWH3000884, BRAWH3001053, BRAWH3001319, BRAWH3001475, BRAWH3001636, BRAWH3001638, BRAWH3001712, BRAWH3001760, BRAWH3001783, BRAWH3001833, BRAWH3001879, BRAWH3001891, BRAWH3001918, BRAWH3002433, BRAWH3002467, BRAWH3002513, BRAWH3002600, BRAWH3002819, BRAWH3003019, BRAWH3003136, BRAWH3003244, BRAWH3003343, BRAWH3003411, BRAWH3003522, BRAWH3003573, BRAWH3003598, BRAWH3003801, BRAWH3003975, BRAWH3004222,

BRAWH3004335, BRAWH3004453, BRAWH3004530, BRAWH3004666, BRAWH3005037, BRAWH3005047, BRAWH3005132, BRAWH3005146, BRAWH3005292, BRAWH3005300, BRAWH3005422, BRAWH3005534, BRAWH3005886, BRAWH3005892, BRAWH3005896, BRAWH3005912, BRAWH3005981, BRAWH3006547, BRAWH3006548, BRAWH3006761, BRAWH3006792, BRAWH3007129, BRAWH3007221, BRAWH3007441, BRAWH3007455, BRAWH3007506, BRAWH3007726, BRAWH3007783, BRAWH3008167, BRAWH3008341, BRAWH3008559, BRAWH3008697, BRAWH3008867, BRAWH3008931, BRAWH3009017, BRAWH3009285, BRAWH3009297, BRAWH3009546, BRAWH3009961, BRAWH3010461, BRAWH3010560, BRAWH3010602, BRAWH3010657, BRAWH3010726, BRAWH3010833, BRAWH3011101, BRAWH3011331, BRAWH3011402, BRAWH3011577, BRAWH3011623,

BRAWH3011637, BRAWH3011685, BRAWH3011907, BRAWH3011929, BRAWH3012005, BRAWH3012662, BRAWH3012779, BRAWH3013009, BRAWH3013049, BRAWH3013197, BRAWH3013264, BRAWH3013508, BRAWH3014609, BRAWH3014639, BRAWH3015017, BRAWH3015175, BRAWH3015260, BRAWH3015610, BRAWH3015825, BRAWH3016123, BRAWH3016715, BRAWH3017259, BRAWH3017260, BRAWH3017477, BRAWH3017537, BRAWH3017980, BRAWH3018063, BRAWH3018369, BRAWH3018548, BRAWH3018969, BRAWH3019026, BRAWH3019529, BRAWH3019594, BRAWH3019629, BRAWH3019820, BRAWH3020200, BRAWH3020318, BRAWH3020486, BRAWH3020884, BRAWH3020928, BRAWH3021012, BRAWH3021574, BRAWH3021580, BRAWH3021641, BRAWH3021643, BRAWH3021724, BRAWH3022347, BRAWH3022431, BRAWH3022459, BRAWH3022542,

BRAWH3022719, BRAWH3022866, BRAWH3022900, BRAWH3023156, BRAWH3023168, BRAWH3023172, BRAWH3023274, BRAWH3023421, BRAWH3024231, BRAWH3024242, BRAWH3024469, BRAWH3024506, BRAWH3024613, BRAWH3024989, BRAWH3026349, BRAWH3026394, BRAWH3027420, BRAWH3027440, BRAWH3027533, BRAWH3027574, BRAWH3027607, BRAWH3027616, BRAWH3027675, BRAWH3027806, BRAWH3027880, BRAWH3027927, BRAWH3028202, BRAWH3028223, BRAWH3028306, BRAWH3028461, BRAWH3028645, BRAWH3028754, BRAWH3028796, BRAWH3029254, BRAWH3029313, BRAWH3029538, BRAWH3029556, BRAWH3030613, BRAWH3030772, BRAWH3030810, BRAWH3030892, BRAWH3030910, BRAWH3030950, BRAWH3031054, BRAWH3031710, BRAWH3031902, BRAWH3032298, BRAWH3032340, BRAWH3032571, BRAWH3032620,

BRAWH3032930, BRAWH3033117, BRAWH3033293, BRAWH3033448, BRAWH3033513, BRAWH3034097, BRAWH3034114, BRAWH3034134, BRAWH3034668, BRAWH3034743, BRAWH3034775, BRAWH3034890, BRAWH3035154, BRAWH3035403, BRAWH3035904, BRAWH3035914, BRAWH3035936, BRAWH3036077, BRAWH3036247, BRAWH3036270, BRAWH3036561, BRAWH3036738, BRAWH3036951, BRAWH3037265, BRAWH3037394, BRAWH3037533, BRAWH3037979, BRAWH3038055, BRAWH3038230, BRAWH3038252, BRAWH3038324, BRCAN1000065, BRCAN1000076, BRCAN1000105, BRCAN1000149, BRCAN1000168, BRCAN2000148, BRCAN2000149, BRCAN2000209, BRCAN2000239, BRCAN2000346, BRCAN2000364, BRCAN2000383, BRCAN2000418, BRCAN2000523, BRCAN2000541, BRCAN2000563, BRCAN2000577, BRCAN2060620, BRCAN2000639,

BRCAN2000763, BRCAN2000832, BRCAN2000923, BRCAN2001223, BRCAN2001866, BRCAN2002173, BRCAN2002416, BRCAN2002473, BRCAN2002562, BRCAN2002662, BRCAN2002826, BRCAN2002854, BRCAN2002856, BRCAN2002892, BRCAN2002944, BRCAN2002948, BRCAN2003070, BRCAN2003269, BRCAN2003389, BRCAN2003628, BRCAN2003703, BRCAN2003746, BRCAN2003814, BRCAN2003987, BRCAN2004173, BRCAN2004355, BRCAN2004371, BRCAN2004653, BRCAN2004699, BRCAN2005436, BRCAN2005449, BRCAN2006019, BRCAN2006051, BRCAN2006063, BRCAN2006117, BRCAN2006174, BRCAN2006290, BRCAN2006297, BRCAN2006323, BRCAN2006401, BRCAN2006411, BRCAN2006450, BRCAN2006955, BRCAN2007119, BRCAN2007144, BRCAN2007409, BRCAN2007426, BRCAN2007525, BRCAN2007700, BRCAN2008494,

BRCAN2008528, BRCAN2008701, BRCAN2008894, BRCAN2009156, BRCAN2009168, BRCAN2009203, BRCAN2010374, BRCAN2010376, BRCAN2010547, BRCAN2010581, BRCAN2010665, BRCAN2011254, BRCAN2011316, BRCAN2011602, BRCAN2012110, BRCAN2012355, BRCAN2012402, BRCAN2012408, BRCAN2012481, BRCAN2012613, BRCAN2012713, BRCAN2012929, BRCAN2013655, BRCAN2013660, BRCAN2014143, BRCAN2014229, BRCAN2014370, BRCAN2014602, BRCAN2014652, BRCAN2014788, BRCAN2014881, BRCAN2014952, BRCAN2015371, BRCAN2015402, BRCAN2015464, BRCAN2015719, BRCAN2015757, BRCAN2015886, BRCAN2016025, BRCAN2016046, BRCAN2016174, BRCAN2016433, BRCAN2016619, BRCAN2016762, BRCAN2016814, BRCAN2017442, BRCAN2017905, BRCAN2018240, BRCAN2018269, BRCAN2018490,

BRCAN2018566, BRCAN2018667, BRCAN2018748, BRCAN2018935, BRCAN2019002, BRCAN2019387, BRCAN2019653, BRCAN2019773, BRCAN2019907, BRCAN2020234, BRCAN2020331, BRCAN2020412, BRCAN2020467, BRCAN2020710, BRCAN2020880, BRCAN2021028, BRCAN2021325, BRCAN2021452, BRCAN2021669, BRCAN2021718, BRCAN2022191, BRCAN2022472, BRCAN2022474, BRCAN2023347, BRCAN2023724, BRCAN2024451, BRCAN2024563, BRCAN2025009, BRCAN2025093, BRCAN2025349, BRCAN2025368, BRCAN2025712, BRCAN2025982, BRCAN2026117, BRCAN2026197, BRCAN2026340, BRCAN2026711, BRCAN2027150, BRCAN2027310, BRCAN2027334, BRCAN2027355, BRCAN2027364, BRCAN2027513, BRCAN2027593, BRCAN2027970, BRCAN2028021, BRCAN2028036, BRCAN2028040, BRCAN2028319, BRCAN2028338,

BRCAN2028378, BRCAN2028460, BRCAN2028545, BRCAN2028634, BRCAN2028702, BRCOC1000087, BRCOC1000094, BRCOC2000004, BRCOC2000047, BRCOC2000152, BRCOC2000184, BRCOC2000186, BRCOC2000360, BRCOC2000404, BRCOC2000487, BRCOC2000637, BRCOC2000670, BRCOC2000816, BRCOC2000850, BRCOC2001355, BRCOC2002085, BRCOC2002155, BRCOC2002323, BRCOC2002659, BRCOC2002664, BRCOC2002686, BRCOC2002751, BRCOC2002756, BRCOC2002777, BRCOC2002835, BRCOC2003100, BRCOC2003125, BRCOC2003213, BRCOC2003513, BRCOC2003732, BRCOC2003740, BRCOC2004036, BRCOC2004175, BRCOC2004354, BRCOC2004455, BRCOC2004950, BRCOC2005542, BRCOC2005903, BRCOC2005951, BRCOC2006164, BRCOC2006189, BRCOC2006243, BRCOC2006270, BRCOC2006639, BRCOC2006942,

BRCOC2007034, BRCOC2007593, BRCOC2007733, BRCOC2007769, BRCOC2008064, BRCOC2008225, BRCOC2008642, BRCOC2008993, BRCOC2009052, BRCOC2009196, BRCOC2009380, BRCOC2009638, BRCOC2009937, BRCOC2010115, BRCOC2010123, BRCOC2010510, BRCOC2010980, BRCOC2010994, BRCOC2011010, BRCOC2011398, BRCOC2011418, BRCOC2011506, BRCOC2011769, BRCOC2011996, BRCOC2012229, BRCOC2012386, BRCOC2012813, BRCOC2013448, BRCOC2013573, BRCOC2013675, BRCOC2013780, BRCOC2014013, BRCOC2014033, BRCOC2014400, BRCOC2014748, BRCOC2014833, BRCOC2015824, BRCOC2015908, BRCOC2015944, BRCOC2016525, BRCOC2016661, BRCOC2017061, BRCOC2017652, BRCOC2017856, BRCOC2018775, BRCOC2018877, BRCOC2019255, BRCOC2019549, BRCOC2019841, BRCOC2019896,

BRCOC2019903, BRCOC2019910, BRCOC2019934, BRCOC2020122, BRCOC2020142, BRHIP1000058, BRHIP1000072, BRHIP1000104, BRHIP1000108, BRHIP1000129, BRHIP1000174, BRHIP1000258, BRHIP2000021, BRHIP2000079, BRHIP2000087, BRHIP2000163, BRHIP2000210, BRHIP2000239, BRHIP2000245, BRHIP2000312, BRHIP2000359, BRHIP2000445, BRHIP2000506, BRHIP2000532, BRHIP2000534, BRHIP2000553, BRHIP2000621, BRHIP2000691, BRHIP2000819, BRHIP2000826, BRHIP2000920, BRHIP2001074, BRHIP2001686, BRHIP2001782, BRHIP2001802, BRHIP2001805, BRHIP2001886, BRHIP2001927, BRHIP2002122, BRHIP2002143, BRHIP2002172, BRHIP2002339, BRHIP2002346, BRHIP2002510, BRHIP2002664, BRHIP2002722, BRHIP2002929, BRHIP2003048, BRHIP2003242, BRHIP2003272,

BRHIP2003748, BRHIP2003786, BRHIP2003828, BRHIP2003917, BRHIP2004312, BRHIP2004355, BRHIP2004359, BRHIP2004814, BRHIP2004883, BRHIP2004902, BRHIP2005010, BRHIP2005236, BRHIP2005271, BRHIP2005354, BRHIP2005600, BRHIP2005719, BRHIP2005724, BRHIP2005752, BRHIP2005932, BRHIP2006617, BRHIP2006782, BRHIP2006800, BRHIP2006819, BRHIP2006921, BRHIP2007307, BRHIP2007616, BRHIP2007690, BRHIP2007741, BRHIP2007928, BRHIP2007969, BRHIP2008269, BRHIP2008389, BRHIP2008607, BRHIP2008756, BRHIP2008909, BRHIP2009019, BRHIP2009177, BRHIP2009340, BRHIP2009351, BRHIP2009414, BRHIP2009474, BRHIP2009617, BRHIP2009685, BRHIP2010217, BRHIP2010309, BRHIP2010444, BRHIP2010593, BRHIP2010610, BRHIP2010723, BRHIP2010744,

BRHIP2011080, BRHIP2011120, BRHIP2011199, BRHIP2011508, BRHIP2011576, BRHIP2011783, BRHIP2011838, BRHIP2011891, BRHIP2011933, BRHIP2012054, BRHIP2012059, BRHIP2012141, BRHIP2012314, BRHIP2012360, BRHIP2012545, BRHIP2012580, BRHIP2012627, BRHIP2012642, BRHIP2012750, BRHIP2012922, BRHIP2012923, BRHIP2012972, BRHIP2013286, BRHIP2013447, BRHIP2013510, BRHIP2013699, BRHIP2013723, BRHIP2013958, BRHIP2013972, BRHIP2014063, BRHIP2014228, BRHIP2014285, BRHIP2014291, BRHIP2014373, BRHIP2014386, BRHIP2014387, BRHIP2014954, BRHIP2015153, BRHIP2015224, BRHIP2015356, BRHIP2015360, BRHIP2015679, BRHIP2016005, BRHIP2016125, BRHIP2016788, BRHIP2016968, BRHIP2016990, BRHIP2017010, BRHIP2017315, BRHIP2017385,

BRHIP2017404, BRHIP2017542, BRHIP2017553, BRHIP2017642, BRHIP2017714, BRHIP2017780, BRHIP2017945, BRHIP2018014, BRHIP2018369, BRHIP2018612, BRHIP2018635, BRHIP2018650, BRHIP2018719, BRHIP2018805, BRHIP2018998, BRHIP2019007, BRHIP2019047, BRHIP2019177, BRHIP2019186, BRHIP2019291, BRHIP2019494, BRHIP2019589, BRHIP2019641, BRHIP2019819, BRHIP2019884, BRHIP2019972, BRHIP2020182, BRHIP2020509, BRHIP2020573, BRHIP2020622, BRHIP2020695, BRHIP2020743, BRHIP2020842, BRHIP2020859, BRHIP2020930, BRHIP2021102, BRHIP2021289, BRHIP2021495, BRHIP2021615, BRHIP2021744, BRHIP2021858, BRHIP2021870, BRHIP2021929, BRHIP2022006, BRHIP2022054, BRHIP2022221, BRHIP2022298, BRHIP2022467, BRHIP2022708, BRHIP2022986,

BRHIP2023071, BRHIP2023229, BRHIP2023272, BRHIP2023292, BRHIP2023309, BRHIP2023369, BRHIP2023438, BRHIP2023695, BRHIP2023735, BRHIP2023762, BRHIP2023773, BRHIP2023860, BRHIP2023869, BRHIP2024046, BRHIP2024165, BRHIP2024201, BRHIP2024347, BRHIP2024398, BRHIP2024549, BRHIP2024789, BRHIP2024941, BRHIP2025245, BRHIP2025366, BRHIP2025448, BRHIP2025679, BRHIP2025739, BRHIP2025797, BRHIP2026021, BRHIP2026061, BRHIP2026155, BRHIP2026214, BRHIP2026288, BRHIP2026346, BRHIP2026557, BRHIP2026723, BRHIP2026877, BRHIP2027054, BRHIP2027077, BRHIP2027563, BRHIP2027685, BRHIP2027762, BRHIP2028187, BRHIP2028303, BRHIP2028330, BRHIP2028480, BRHIP2028583, BRHIP2028593, BRHIP2029176, BRHIP2029283, BRHIP2029393,

BRHIP2029529, BRHIP2029643, BRHIP2029663, BRHIP3000060, BRHIP3000070, BRHIP3000081, BRHIP3000111, BRHIP3000131, BRHIP3000240, BRHIP3000457, BRHIP3000475, BRHIP3000488, BRHIP3000526, BRHIP3000626, BRHIP3000859, BRHIP3000907, BRHIP3001079, BRHIP3001141, BRHIP3001283, BRHIP3001338, BRHIP3001360, BRHIP3001481, BRHIP3001878, BRHIP3001964, BRHIP3002114, BRHIP3002124, BRHIP3002363, BRHIP3002691, BRHIP3002920, BRHIP3002931, BRHIP3003063, BRHIP3003126, BRHIP3003306, BRHIP3003340, BRHIP3003395, BRHIP3003484, BRHIP3003644, BRHIP3003688, BRHIP3003795, BRHIP3003961, BRHIP3003984, BRHIP3004193, BRHIP3004215, BRHIP3004416, BRHIP3004710, BRHIP3004725, BRHIP3004774, BRHIP3004786, BRHIP3004863, BRHIP3004968,

BRHIP3005037, BRHIP3005137, BRHIP300-5142, BRHIP3005231, BRHIP3005574, BRHIP3005673, BRHIP3005768, BRHIP3005801, BRHIP3005944, BRHIP3006279, BRHIP3006294, BRHIP3006390, BRHIP3006449, BRHIP3006683, BRHIP3006717, BRHIP3006786, BRHIP3006950, BRHIP3007172, BRHIP3007183, BRHIP3007195, BRHIP3007239, BRHIP3007291, BRHIP3007409, BRHIP3007424, BRHIP3007483, BRHIP3007609, BRHIP3007640, BRHIP3007960, BRHIP3008082, BRHIP3008183, BRHIP3008314, BRHIP3008320, BRHIP3008405, BRHIP3008598, BRHIP3008606, BRHIP3008691, BRHIP3008714, BRHIP3008997, BRHIP3009099, BRHIP3009140, BRHIP3009181, BRHIP3009318, BRHIP3009427, BRHIP3009448, BRHIP3009672, BRHIP3009753, BRHIP3010135, BRHIP3010289, BRHIP3010529, BRHIP3011082,

BRHIP3011241, BRHIP3011269, BRHIP3011460, BRHIP3011567, BRHIP3011831, BRHIP3012185, BRHIP3012289, BRHIP3012357, BRHIP3012736, BRHIP3012745, BRHIP3012868, BRHIP3013078, BRHIP3013271, BRHIP3013588, BRHIP3013698, BRHIP3013765, BRHIP3013897, BRHIP3014237, BRHIP3014675, BRHIP3015620, BRHIP3015751, BRHIP3015854, BRHIP3016032, BRHIP3016213, BRHIP3016302, BRHIP3016421, BRHIP3017109, BRHIP3017146, BRHIP3017256, BRHIP3017325, BRHIP3017558, BRHIP3017637, BRHIP3018278, BRHIP3018784, BRHIP3019643, BRHIP3019824, BRHIP3019880, BRHIP3019956, BRHIP3020155, BRHIP3020182, BRHIP3020733, BRHIP3021019, BRHIP3021499, BRHIP3021534, BRHIP3021778, BRHIP3021987, BRHIP3022656, BRHIP3023922, BRHIP3024118, BRHIP3024533,

BRHIP3024703, BRHIP3024725, BRHIP3024820, BRHIP3025161, BRHIP3025702, BRHIP3025795, BRHIP3025844, BRHIP3026052, BRHIP3026097, BRHIP3026231, BRHIP3026335, BRHIP3026651, BRHIP3027105, BRHIP3027137, BRHIP3027160, BRHIP3027191, BRHIP3027501, BRHIP3027594, BRHIP3027651, BRHIP3027854, BRHIP3027947, BRHIP3028570, BRHIP3028742, BRSSN1000056, BRSSN1000059, BRSSN1000092, BRSSN1000153, BRSSN2000097, BRSSN2000175, BRSSN2000197, BRSSN2000244, BRSSN2000293, BRSSN2000312, BRSSN2000518, BRSSN2000561, BRSSN2000566, BRSSN2000684, BRSSN2000715, BRSSN2000832, BRSSN2001275, BRSSN2001342, BRSSN2001426, BRSSN2001503, BRSSN2001579, BRSSN2001758, BRSSN2001810, BRSSN2002857, BRSSN2002859, BRSSN2003012, BRSSN2003086,

BRSSN2003093, BRSSN2003820, BRSSN2003841, BRSSN2003937, BRSSN2004142, BRSSN2004303, BRSSN2004304, BRSSN2004496, BRSSN2004657, BRSSN2004679, BRSSN2004686, BRSSN2004710, BRSSN2004719, BRSSN2005847, BRSSN2006133, BRSSN2006390, BRSSN2006575, BRSSN2006611, BRSSN2006644, BRSSN2007076, BRSSN2007168, BRSSN2007202, BRSSN2007338, BRSSN2007464, BRSSN2007504, BRSSN2007819, BRSSN2008125, BRSSN2008419, BRSSN2008464, BRSSN2008549, BRSSN2008797, BRSSN2009119, BRSSN2009378, BRSSN2009389, BRSSN2009395, BRSSN2009627, BRSSN2009630, BRSSN2009702, BRSSN2010110, BRSSN2010587, BRSSN2010596, BRSSN2010830, BRSSN2011262, BRSSN2011653, BRSSN2011738, BRSSN2011843, BRSSN2012081, BRSSN2012103, BRSSN2012157, BRSSN2012198,

BRSSN2012254, BRSSN2012439, BRSSN2012622, BRSSN2012691, BRSSN2012838, BRSSN2013095, BRSSN2013544, BRSSN2013696, BRSSN2013702, BRSSN2013733, BRSSN2013753, BRSSN2013874, BRSSN2014112, BRSSN2014294, BRSSN2014299, BRSSN2014424, BRSSN2014513, BRSSN2014556, BRSSN2014573, BRSSN2014610, BRSSN2014636, BRSSN2015199, BRSSN2015238, BRSSN2015369, BRSSN2015493, BRSSN2015497, BRSSN2015554, BRSSN2015707, BRSSN2015773, BRSSN2015907, BRSSN2015982, BRSSN2016589, BRSSN2016768, BRSSN2016905, BRSSN2017297, BRSSN2017530, BRSSN2017682, BRSSN2017757, BRSSN2018218, BRSSN2018440, BRSSN2018447, BRSSN2018581, BRSSN2018731, BRSSN2018771, BRSSN2018925, BRSTN1000083, BRSTN1000097, BRSTN2000058, BRSTN2000070, BRSTN2000220,

BRSTN2000312, BRSTN2000367, BRSTN2000536, BRSTN2000872, BRSTN2001613, BRSTN2001801, BRSTN2002105, BRSTN2002400, BRSTN2002532, BRSTN2002691, BRSTN2003590, BRSTN2003835, BRSTN2004863, BRSTN2004869, BRSTN2004987, BRSTN2005721, BRSTN2006306, BRSTN2006466, BRSTN2006580, BRSTN2006583, BRSTN2006638, BRSTN2006865, BRSTN2007000, BRSTN2007118, BRSTN2007284, BRSTN2007765, BRSTN2008052, BRSTN2008283, BRSTN2008367, BRSTN2008418, BRSTN2008457, BRSTN2008475, BRSTN2008930, BRSTN2009247, BRSTN2010107, BRSTN2010363, BRSTN2010416, BRSTN2010500, BRSTN2010569, BRSTN2010750, BRSTN2010923, BRSTN2011211, BRSTN2011597, BRSTN2011688, BRSTN2011799, BRSTN2012089, BRSTN2012284, BRSTN2012320, BRSTN2012380, BRSTN2012451,

BRSTN2013171, BRSTN2013354, BRSTN2014490, BRSTN2014633, BRSTN2014751, BRSTN2014770, BRSTN2015015, BRSTN2015889, BRSTN2015978, BRSTN2016066, BRSTN2016335, BRSTN2016678, BRSTN2016918, BRSTN2016992, BRSTN2017104, BRSTN2017151, BRSTN2017237, BRSTN2017379, BRSTN2017761, BRSTN2017771, BRSTN2017995, BRSTN2018077, BRSTN2018083, BRSTN2018596, BRSTN2018692, BRSTN2018712, BRSTN2019079, BRSTN2019129, BRTHA1000021, BRTHA1000063, BRTHA1000311, BRTHA2000474, BRTHA2000514, BRTHA2000574, BRTHA2000855, BRTHA2000969, BRTHA2000972, BRTHA2001304, BRTHA2001370, BRTHA2001462, BRTHA2001717, BRTHA2001812, BRTHA2002091, BRTHA2002115, BRTHA2002133, BRTHA2002281, BRTHA2002376, BRTHA2002442, BRTHA2002493, BRTHA2002565,

BRTHA2002608, BRTHA2002702, BRTHA2002721, BRTHA2002726, BRTHA2002741, BRTHA2002808, BRTHA2002896, BRTHA2002903, BRTHA2003030, BRTHA2003110, BRTHA2003116, BRTHA2003436, BRTHA2003759, BRTHA2003794, BRTHA2004371, BRTHA2004582, BRTHA2004629, BRTHA2004639, BRTHA2004642, BRTHA2004812, BRTHA2004821, BRTHA2004978, BRTHA2004992, BRTHA2005005, BRTHA2005013, BRTHA2005448, BRTHA2005579, BRTHA2005942, BRTHA2005956, BRTHA2006075, BRTHA2006146, BRTHA2006194, BRTHA2006232, BRTHA2006720, BRTHA2006735, BRTHA2006974, BRTHA2006975, BRTHA2007060, BRTHA2007122, BRTHA2007422, BRTHA2007603, BRTHA2007665, BRTHA2007998, BRTHA2008316, BRTHA2008335, BRTHA2008502, BRTHA2008513, BRTHA2008527, BRTHA2008535, BRTHA2008598,

BRTHA2008669, BRTHA2008955, BRTHA2009311, BRTHA2009349, BRTHA2009486, BRTHA2009803, BRTHA2009846, BRTHA2009886, BRTHA2009972, BRTHA2010008, BRTHA2010033, BRTHA2010073, BRTHA2010097, BRTHA2010191, BRTHA2010397, BRTHA2010672, BRTHA2010884, BRTHA2010907, BRTHA2011062, BRTHA2011194, BRTHA2011321, BRTHA2011351, BRTHA2011500, BRTHA2011641, BRTHA2011691, BRTHA2012129, BRTHA2012189, BRTHA2012351, BRTHA2012392, BRTHA2012466, BRTHA2012555, BRTHA2012562, BRTHA2012980, BRTHA2013054, BRTHA2013262, BRTHA2013275, BRTHA2013426, BRTHA2013460, BRTHA2013515, BRTHA2013540, BRTHA2013595, BRTHA2013610, BRTHA2013707, BRTHA2013942, BRTHA2014287, BRTHA2014334, BRTHA2014381, BRTHA2014578, BRTHA2014647, BRTHA2014663,

BRTHA2014792, BRTHA2014828, BRTHA2014909, BRTHA2014975, BRTHA2014997, BRTHA2015406, BRTHA2015478, BRTHA2015696, BRTHA2015700, BRTHA2015853, BRTHA2015878, BRTHA2016179, BRTHA2016215, BRTHA2016267, BRTHA2016318, BRTHA2016496, BRTHA2016543, BRTHA2016552, BRTHA2016577, BRTHA2016765, BRTHA2016918, BRTHA2017353, BRTHA2017364, BRTHA2017972, BRTHA2017985, BRTHA2018011, BRTHA2018129, BRTHA2018165, BRTHA2018304, BRTHA2018344, BRTHA2018420, BRTHA2018443, BRTHA2018504, BRTHA2018558, BRTHA2018591, BRTHA2018624, BRTHA2018706, BRTHA2018707, BRTHA2019014, BRTHA2019022, BRTHA2019048, BRTHA2019073, BRTHA2019726, BRTHA2019743, BRTHA2020400, BRTHA2020566, BRTHA2020642, BRTHA2020695, BRTHA2020721, BRTHA2020781,

BRTHA2020811, BRTHA2020910, BRTHA2021212, BRTHA2021440, BRTHA2021450, BRTHA2022074, BRTHA2022914, BRTHA2022968, BRTHA2023402, BRTHA2023437, BRTHA2024177, BRTHA2024354, BRTHA2024712, BRTHA2025869, BRTHA2026071, BRTHA2026290, BRTHA2026311, BRTHA2027227, BRTHA2027229, BRTHA2027250, BRTHA2027546, BRTHA2028297, BRTHA2029969, BRTHA2030036, BRTHA2030213, BRTHA2031517, BRTHA2031800, BRTHA2031917, BRTHA2032763, BRTHA2032845, BRTHA2033122, BRTHA2033155, BRTHA2033264, BRTHA2033320, BRTHA2033469, BRTHA2033683, BRTHA2034281, BRTHA2034576, BRTHA2034874, BRTHA2035448, BRTHA2035743, BRTHA2036055, BRTHA2036295, BRTHA2036660, BRTHA2037247, BRTHA2038279, BRTHA2038345, BRTHA2038353, BRTHA2038396, BRTHA3000252,

BRTHA3000273, BRTHA3000296, BRTHA3000297, BRTHA3000325, BRTHA3000456, BRTHA3000633, BRTHA3000789, BRTHA3001221, BRTHA3001623, BRTHA3002265, BRTHA3002401, BRTHA3002411, BRTHA3002427, BRTHA3002864, BRTHA3002933, BRTHA3002955, BRTHA3003000, BRTHA3003225, BRTHA3003339, BRTHA3003343, BRTHA3003417, BRTHA3003449, BRTHA3003474, BRTHA3003490, BRTHA3003704, BRTHA3004432, BRTHA3004475, BRTHA3004502, BRTHA3004610, BRTHA3005046, BRTHA3005735, BRTHA3005988, BRTHA3006318, BRTHA3006593, BRTHA3006856, BRTHA3007113, BRTHA3007130, BRTHA3007148, BRTHA3007319, BRTHA3007469, BRTHA3007472, BRTHA3007532, BRTHA3007662, BRTHA3007685, BRTHA3007769, BRTHA3007922, BRTHA3008143, BRTHA3008293, BRTHA3008310, BRTHA3008386,

BRTHA3008520, BRTHA3008608, BRTHA3008694, BRTHA3008778, BRTHA3008826, BRTHA3009037, BRTHA3009090, BRTHA3009291, BRTHA3009858, BRTHA3010135, BRTHA3010366, BRTHA3010469, BRTHA3010530, BRTHA3010540, BRTHA3010717, BRTHA3011149, BRTHA3011187, BRTHA3011194, BRTHA3011229, BRTHA3011361, BRTHA3011510, BRTHA3011892, BRTHA3012265, BRTHA3013860, BRTHA3013882, BRTHA3013884, BRTHA3013981, BRTHA3014000, BRTHA3014105, BRTHA3014507, BRTHA3014547, BRTHA3014691, BRTHA3014835, BRTHA3014854, BRTHA3014908, BRTHA3014920, BRTHA3015815, BRTHA3016616, BRTHA3016835, BRTHA3016845, BRTHA3016917, BRTHA3017047, BRTHA3017589, BRTHA3017848, BRTHA3018409, BRTHA3018514, BRTHA3018617, BRTHA3018623, BRTHA3018656, BRTHA3019048,

BRTHA3019105, BRTHA3019183, BRTHA3020000, BRTHA3020369, BRTHA3021569, BRTHA3021708, BRTHA3021786, BRTHA3021971, BRTHA3022641, BRTHA3023523, BRTHA3023590, BRTHA3023929, BRTHA3024233, BRTHA3024600, BRTHA3025073, BRTHA3025789, BRTHA3025826, BRTHA3026161, BRTHA3026180, BRTHA3026556, BRTHA3026916, BRTHA3027171, BRTHA3027318, BRTHA3027638, BRTHA3027820, BRTHA3027957, BRTHA3028339, BRTHA3028505, BRTHA3028782, CD34C2000051, CD34C2000152, CD34C2000175, CD34C2000443, CD34C3000125, CD34C3000252, CD34C3000314, CD34C3000424, CD34C3000494, CERVX1000042, CERVX2000812, CERVX2000968, CERVX2002006, CERVX2002013, CERVX2002430, CERVX2002770, CHONS1000058, CHONS2000172, CHONS2000797, CHONS2001287, CHONS2001382,

CHONS2001797, CHONS2001834, CHONS2002419, COLON1000030, COLON1000084, COLON1000135, COLON2000470, COLON2000557, COLON2000568, COLON2001721, COLON2001829, COLON2001866, COLON2002443, COLON2002520, COLON2003043, COLON2003529, COLON2004318, COLON2004351, COLON2004478, COLON2004911, COLON2005126, COLON2005623, COLON2005735, COLON2005772, COLON2006263, COLON2006282, COLON2006417, COLON2007274, COLON2007733, COLON2008105, COLON2009337, COLON2009499, CORDB1000140, CORDB2000061, CORDB2000127, CORDB2000541, CTONG1000009, CTONG1000010, CTONG1000020, CTONG1000022, CTONG1000040, CTONG1000056, CTONG1000062, CTONG1000086, CTONG1000088, CTONG1000093, CTONG1000097, CTONG1000113, CTONG1000165, CTONG1000175,

CTONG1000181, CTONG1000218, CTONG1000220, CTONG1000283, CTONG1000288, CTONG1000302, CTONG1000341, CTONG1000476, CTONG1000488, CTONG1000508, CTONG1000540, CTONG2000031, CTONG2000034, CTONG2000042, CTONG2000063, CTONG2000201, CTONG2000218, CTONG2000279, CTONG2000303, CTONG2000330, CTONG2000395, CTONG2000452, CTONG2000465, CTONG2000469, CTONG2000480, CTONG2000561, CTONG2000589, CTONG2000766, CTONG2000771, CTONG2000819, CTONG2000827, CTONG2000846, CTONG2000855, CTONG2000977, CTONG2001139, CTONG2001226, CTONG2001320, CTONG2001366, CTONG2001413, CTONG2001428, CTONG2001513, CTONG2001524, CTONG2001533, CTONG2001598, CTONG2001681, CTONG2001724, CTONG2001749, CTONG2001800, CTONG2001820, CTONG2001858,

CTONG2001877, CTONG2001911, CTONG2001955, CTONG2002066, CTONG2002095, CTONG2002143, CTONG2002395, CTONG2002417, CTONG2002418, CTONG2002453, CTONG2002520, CTONG2002558, CTONG2002590, CTONG2002709, CTONG2002721, CTONG2002816, CTONG2002820, CTONG2002832, CTONG2002841, CTONG2002903, CTONG2002965, CTONG2002999, CTONG2003028, CTONG2003094, CTONG2003100, CTONG2003115, CTONG2003189, CTONG2003245, CTONG2003293, CTONG2003350, CTONG2003361, CTONG2003372, CTONG2003375, CTONG2003427, CTONG2003599, CTONG2003680, CTONG2003699, CTONG2003764, CTONG2003782, CTONG2003889, CTONG2003937, CTONG2003945, CTONG2004000, CTONG2004062, CTONG2004115, CTONG2004126, CTONG2004264, CTONG2004305, CTONG2004397, CTONG2004454,

CTONG2004487, CTONG2004550, CTONG2004669, CTONG2004716, CTONG2004912, CTONG2004941, CTONG2004948, CTONG2005028, CTONG2005049, CTONG2005110, CTONG2005265, CTONG2005278, CTONG2005290, CTONG2005374, CTONG2005399, CTONG2005468, CTONG2005553, CTONG2005567, CTONG2005585, CTONG2005635, CTONG2005775, CTONG2005795, CTONG2005890, CTONG2005904, CTONG2005913, CTONG2006004, CTONG2006129, CTONG2006223, CTONG2006273, CTONG2006310, CTONG2006393, CTONG2006412, CTONG2006449, CTONG2006515, CTONG2006524, CTONG2006568, CTONG2006611, CTONG2006620, CTONG2006666, CTONG2006691, CTONG2006709, CTONG2006798, CTONG2006836, CTONG2006942, CTONG2006964, CTONG2007007, CTONG2007009, CTONG2007072, CTONG2007091, CTONG2007104,

CTONG2007168, CTONG2007175, CTONG2007259, CTONG2007272, CTONG2007293, CTONG2007297, CTONG2007399, CTONG2007400, CTONG2007417, CTONG2007441, CTONG2007474, CTONG2007586, CTONG2007613, CTONG2007623, CTONG2007681, CTONG2007776, CTONG2007779, CTONG2007898, CTONG2007959, CTONG2007970, CTONG2008014, CTONG2008184, CTONG2008262, CTONG2008269, CTONG2008321, CTONG2008402, CTONG2008405, CTONG2008466, CTONG2008506, CTONG2008521, CTONG2008562, CTONG2008577, CTONG2008595, CTONG2008621, CTONG2008720, CTONG2008721, CTONG2008773, CTONG2008792, CTONG2008989, CTONG2009033, CTONG2009108, CTONG2009132, CTONG2009156, CTONG2009181, CTONG2009257, CTONG2009258, CTONG2009268, CTONG2009270, CTONG2009395, CTONG2009423,

CTONG2009440, CTONG2009527, CTONG2009531, CTONG2009534, CTONG2009570, CTONG2009572, CTONG2009604, CTONG2009643, CTONG2009675, CTONG2009689, CTONG2009725, CTONG2009766, CTONG2009768, CTONG2009844, CTONG2009871, CTONG2009887, CTONG2009891, CTONG2009955, CTONG2009958, CTONG2009963, CTONG2010018, CTONG2010024, CTONG2010116, CTONG2010133, CTONG2010148, CTONG2010276, CTONG2010330, CTONG2010508, CTONG2010566, CTONG2010623, CTONG2010652, CTONG2010803, CTONG2011188, CTONG2011429, CTONG2011474, CTONG2011676, CTONG2011716, CTONG2011765, CTONG2011779, CTONG2011815, CTONG2011822, CTONG2011920, CTONG2011985, CTONG2012001, CTONG2012029, CTONG2012050, CTONG2012077, CTONG2012101, CTONG2012123, CTONG2012158,

CTONG2012230, CTONG2012401, CTONG2012422, CTONG2012425, CTONG2012447, CTONG2012453, CTONG2012473, CTONG2012476, CTONG2012533, CTONG2012554, CTONG2012564, CTONG2012607, CTONG2012661, CTONG2012724, CTONG2012738, CTONG2012745, CTONG2012758, CTONG2012843, CTONG2012901, CTONG2013149, CTONG2013178, CTONG2013222, CTONG2013284, CTONG2013339, CTONG2013348, CTONG2013352, CTONG2013381, CTONG2013393, CTONG2013511, CTONG2013630, CTONG2013803, CTONG2013985, CTONG2013986, CTONG2014032, CTONG2014058, CTONG2014119, CTONG2014165, CTONG2014191, CTONG2014206, CTONG2014234, CTONG2014264, CTONG2014280, CTONG2014389, CTONG2014491, CTONG2014630, CTONG2014635, CTONG2014646, CTONG2014705, CTONG2014946, CTONG2014954,

CTONG2014959, CTONG2014966, CTONG2014995, CTONG2015091, CTONG2015204, CTONG2015316, CTONG2015330, CTONG2015358, CTONG2015434, CTONG2015518, CTONG2015540, CTONG2015596, CTONG2015633, CTONG2015678, CTONG2015717, CTONG2015804, CTONG2015815, CTONG2015866, CTONG2015914, CTONG2015953, CTONG2016185, CTONG2016217, CTONG2016355, CTONG2016499, CTONG2016505, CTONG2016559, CTONG2016658, CTONG2016775, CTONG2016797, CTONG2016824, CTONG2016846, CTONG2016904, CTONG2016931, CTONG2016942, CTONG2016953, CTONG2017021, CTONG2017292, CTONG2017409, CTONG2017444, CTONG2017458, CTONG2017500, CTONG2017604, CTONG2017650, CTONG2017798, CTONG2017804, CTONG2017814, CTONG2017910, CTONG2017939, CTONG2017989, CTONG2018062,

CTONG2018069, CTONG2018147, CTONG2018217, CTONG2018279, CTONG2018334, CTONG2018343, CTONG2018379, CTONG2018383, CTONG2018413, CTONG2018483, CTONG2018629, CTONG2018632, CTONG2018652, CTONG2018653, CTONG2018655, CTONG2018735, CTONG2018739, CTONG2018808, CTONG2018843, CTONG2018900, CTONG2018976, CTONG2019029, CTONG2019063, CTONG2019144, CTONG2019232, CTONG2019248, CTONG2019400, CTONG2019590, CTONG2019652, CTONG2019704, CTONG2019788, CTONG2019833, CTONG2020031, CTONG2020108, CTONG2020127, CTONG2020171, CTONG2020374, CTONG2020445, CTONG2020522, CTONG2020582, CTONG2020638, CTONG2020806, CTONG2020831, CTONG2020974, CTONG2021024, CTONG2021132, CTONG2021168, CTONG2021289, CTONG2022153, CTONG2022601,

CTONG2022835, CTONG2023021, CTONG2023449, CTONG2023512, CTONG2024206, CTONG2024614, CTONG2024749, CTONG2025496, CTONG2025513, CTONG2025516, CTONG2025610, CTONG2025900, CTONG2026513, CTONG2026770, CTONG2026920, CTONG2026987, CTONG2027150, CTONG2027327, CTONG2027361, CTONG2027591, CTONG2027736, CTONG2027783, CTONG2027828, CTONG2027959, CTONG2028097, CTONG2028208, CTONG2028309, CTONG2028486, CTONG2028518, CTONG2028687, CTONG2028758, CTONG3000657, CTONG3000686, CTONG3000707, CTONG3000896, CTONG3001420, CTONG3001501, CTONG3001560, CTONG3001605, CTONG3001746, CTONG3002020, CTONG3002127, CTONG3002370, CTONG3002412, CTONG3002433, CTONG3002552, CTONG3002588, CTONG3002674, CTONG3002728, CTONG3002835,

CTONG3002909, CTONG3002947, CTONG3002983, CTONG3003165, CTONG3003179, CTONG3003483, CTONG3003553, CTONG3003598, CTONG3003652, CTONG3003654, CTONG3003669, CTONG3003737, CTONG3003905, CTONG3003972, CTONG3004072, CTONG3004397, CTONG3004550, CTONG3004576, CTONG3004607, CTONG3004712, CTONG3004726, CTONG3005325, CTONG3005648, CTONG3005713, CTONG3005803, CTONG3005813, CTONG3006067, CTONG3006073, CTONG3006186, CTONG3006629, CTONG3006650, CTONG3006659, CTONG3007244, CTONG3007444, CTONG3007528, CTONG3007586, CTONG3007870, CTONG3008223, CTONG3008252, CTONG3008258, CTONG3008457, CTONG3008496, CTONG3008566, CTONG3008639, CTONG3008831, CTONG3008951, CTONG3008959, CTONG3009227, CTONG3009287, CTONG3009328,

CTONG3009385, D3OST1000066, D3OST1000109, D3OST1000238, D3OST1000267, D3OST1000270, D3OST2000184, D3OST2000618, D3OST2000654, D3OST2000734, D3OST2001328, D3OST2001669, D3OST2001670, D3OST2001788, D3OST2002068, D3OST2002182, D3OST2002223, D3OST2002262, D3OST2002272, D3OST2002417, D3OST2002436, D3OST2002648, D3OST2003024, D3OST2003331, D3OST2003607, D3OST2003797, D3OST2004637, D3OST3000169, D3OST3000195, D3OST3000291, D3OST3000388, D3OST3000493, D6OST2000127, D6OST2000169, D6OST2000245, D6OST2000358, D6OST2000507, D9OST2000031, D9OST2000278, D9OST2000440, D9OST2000869, D9OST2001319, D9OST2001433, D9OST2002397, D9OST2002673, D9OST2003106, D9OST2003108, D9OST2003137, D9OST2003397, D9OST2003580,

D9OST2003594, D9OST2003762, D9OST2003989, D9OST2004018, D9OST2004073, D9OST2004417, DFNES1000003, DFNES1000083, DFNES1000107, DFNES1000150, DFNES2000143, DFNES2000146, DFNES2000153, DFNES2000212, DFNES2000268, DFNES2000292, DFNES2000335, DFNES2000432, DFNES2000457, DFNES2000471, DFNES2000526, DFNES2000913, DFNES2001091, DFNES2001096, DFNES2001108, DFNES2001177, DFNES2001404, DFNES2001564, DFNES2001565, DFNES2001647, DFNES2001800, DFNES2002220, DFNES2002509, DFNES2002550, DFNES2002588, DFNES2002817, DFNES2002966, DFNES2003081, DFNES2003192, DFNES2003255, DFNES2003742, DFNES2004346, DFNES2004354, DFNES2004371, DFNES2004383, DFNES2004608, DFNES2004684, DFNES2005266, DFNES2005527, DFNES2005540,

DFNES2005690, DFNES2005766, DFNES2005779, DFNES2005875, DFNES2006944, DFNES2007113, DFNES2007299, DFNES2007332, DFNES2007641, DFNES2007757, DFNES2008088, DFNES2008160, DFNES2008280, DFNES2008881, DFNES2009482, DFNES2010502, DFNES2011192, DFNES2011221, DFNES2011239, DFNES2011245, DFNES2011380, DFNES2011499, ERLTF1000021, ERLTF2000324, ERLTF2000465, FCBBF1000023, FCBBF1000025, FCBBF1000038, FCBBF1000053, FCBBF1000063, FCBBF1000069, FCBBF1000077, FCBBF1000115, FCBBF1000117, FCBBF1000118, FCBBF1000121, FCBBF1000125, FCBBF1000131, FCBBF1000139, FCBBF1000155, FCBBF1000157, FCBBF1000171, FCBBF1000174, FCBBF1000198, FCBBF1000206, FCBBF1000220, FCBBF1000232, FCBBF1000233, FCBBF1000243, FCBBF1000270,

FCBBF1000272, FCBBF1000280, FCBBF1000297, FCBBF1000305, FCBBF1000314, FCBBF1000322, FCBBF1000349, FCBBF1000374, FCBBF1000376, FCBBF1000377, FCBBF1000395, FCBBF1000397, FCBBF1000403, FCBBF1000437, FCBBF1000449, FCBBF1000476, FCBBF1000506, FCBBF1000509, FCBBF1000546, FCBBF1000550, FCBBF1000556, FCBBF1000563, FCBBF1000581, FCBBF1000582, FCBBF1000598, FCBBF1000604, FCBBF1000618, FCBBF1000627, FCBBF1000671, FCBBF1000675, FCBBF1000684, FCBBF1000686, FCBBF1000687, FCBBF1000691, FCBBF1000732, FCBBF1000748, FCBBF2000038, FCBBF2000094, FCBBF2000105, FCBBF2000123, FCBBF2000195, FCBBF2000199, FCBBF2000214, FCBBF2000232, FCBBF2000259, FCBBF2000276, FCBBF2000291, FCBBF2000362, FCBBF2000379, FCBBF2000473,

FCBBF2000502, FCBBF2000566, FCBBF2000576, FCBBF2000677, FCBBF2000678, FCBBF2000733, FCBBF2000782, FCBBF2000808, FCBBF2000815, FCBBF2000825, FCBBF2000940, FCBBF2000951, FCBBF2001088, FCBBF2001105, FCBBF2001116, FCBBF2001183, FCBBF2001188, FCBBF2001211, FCBBF2001238, FCBBF2001243, FCBBF2001299, FCBBF2001309, FCBBF2001400, FCBBF2001427, FCBBF2001480, FCBBF2001529, FCBBF2001538, FCBBF2001594, FCBBF2001672, FCBBF2001675, FCBBF2001718, FCBBF2001720, FCBBF2001817, FCBBF2001868, FCBBF2002044, FCBBF2002111, FCBBF2002281, FCBBF2002349, FCBBF2002370, FCBBF2002541, FCBBF2002626, FCBBF2002801, FCBBF2002897, FCBBF2002898, FCBBF2002904, FCBBF2002928, FCBBF2002953, FCBBF2003083, FCBBF2003102, FCBBF2003269,

FCBBF2003293, FCBBF2003297, FCBBF2003395, FCBBF2003528, FCBBF2003543, FCBBF2003549, FCBBF2003567, FCBBF2003636, FCBBF2003823, FCBBF2003895, FCBBF2004066, FCBBF2004090, FCBBF2004138, FCBBF2004217, FCBBF2004256, FCBBF2004373, FCBBF2004448, FCBBF2004671, FCBBF2004707, FCBBF2004788, FCBBF2005069, FCBBF2005122, FCBBF2005428, FCBBF2005439, FCBBF2005538, FCBBF2005637, FCBBF2005645, FCBBF2005658, FCBBF2005733, FCBBF2005909, FCBBF2005966, FCBBF2006131, FCBBF2006418, FCBBF2006452, FCBBF2006634, FCBBF2006781, FCBBF2007012, FCBBF2007186, FCBBF2007265, FCBBF2007302, FCBBF2007556, FCBBF2007610, FCBBF2007633, FCBBF2007840, FCBBF3000102, FCBBF3000110, FCBBF3000120, FCBBF3000168, FCBBF3000175, FCBBF3000227,

FCBBF3000228, FCBBF3000229, FCBBF3000233, FCBBF3000269, FCBBF3000361, FCBBF3000367, FCBBF3000388, FCBBF3000434, FCBBF3000462, FCBBF3000473, FCBBF3000501, FCBBF3000518, FCBBF3000550, FCBBF3000847, FCBBF3001018, FCBBF3001020, FCBBF3001081, FCBBF3001235, FCBBF3001281, FCBBF3001302, FCBBF3001377, FCBBF3001594, FCBBF3001632, FCBBF3001657, FCBBF3001818, FCBBF3001855, FCBBF3001890, FCBBF3001912, FCBBF3001914, FCBBF3001918, FCBBF3001924, FCBBF3001977, FCBBF3002163, FCBBF3002188, FCBBF3002190, FCBBF3002268, FCBBF3002475, FCBBF3002556, FCBBF3002592, FCBBF3002658, FCBBF3002782, FCBBF3002818, FCBBF3002925, FCBBF3003216, FCBBF3003305, FCBBF3003373, FCBBF3003435, FCBBF3003475, FCBBF3003481, FCBBF3003557,

FCBBF3003787, FCBBF3003902, FCBBF3004021, FCBBF3004041, FCBBF3004261, FCBBF3004323, FCBBF3004390, FCBBF3004409, FCBBF3004502, FCBBF3004715, FCBBF3004842, FCBBF3004955, FCBBF3005160, FCBBF3005218, FCBBF3005320, FCBBF3005330, FCBBF3005444, FCBBF3005497, FCBBF3005698, FCBBF3005729, FCBBF3006171, FCBBF3006249, FCBBF3006288, FCBBF3006399, FCBBF3006628, FCBBF3007077, FCBBF3007150, FCBBF3007152, FCBBF3007242, FCBBF3007248, FCBBF3007453, FCBBF3007462, FCBBF3007540, FCBBF3007597, FCBBF3007604, FCBBF3007631, FCBBF3007829, FCBBF3007855, FCBBF3007860, FCBBF3008073, FCBBF3008100, FCBBF3008159, FCBBF3008251, FCBBF3008311, FCBBF3008362, FCBBF3008382, FCBBF3008475, FCBBF3008556, FCBBF3008644, FCBBF3008689,

FCBBF3008748, FCBBF3008870, FCBBF3008938, FCBBF3008944, FCBBF3009069, FCBBF3009101, FCBBF3009152, FCBBF3009256, FCBBF3009317, FCBBF3009428, FCBBF3009464, FCBBF3009479, FCBBF3009526, FCBBF3009541, FCBBF3009703, FCBBF3009717, FCBBF3009888, FCBBF3009949, FCBBF3009978, FCBBF3010008, FCBBF3010012, FCBBF3010124, FCBBF3010130, FCBBF3010142, FCBBF3010149, FCBBF3010211, FCBBF3010361, FCBBF3010508, FCBBF3010544, FCBBF3010586, FCBBF3010601, FCBBF3010665, FCBBF3010729, FCBBF3010733, FCBBF3011003, FCBBF3011418, FCBBF3011485, FCBBF3011523, FCBBF3011570, FCBBF3011592, FCBBF3011867, FCBBF3011889, FCBBF3012170, FCBBF3012182, FCBBF3012288, FCBBF3012332, FCBBF3012347, FCBBF3012443, FCBBF3012588, FCBBF3012667,

FCBBF3012842, FCBBF3012901, FCBBF3013041, FCBBF3013058, FCBBF3013205, FCBBF3013266, FCBBF3013307, FCBBF3013341, FCBBF3013506, FCBBF3013589, FCBBF3013623, FCBBF3013800, FCBBF3013846, FCBBF3014229, FCBBF3014260, FCBBF3014355, FCBBF3014567, FCBBF3014770, FCBBF3015100, FCBBF3015119, FCBBF3015131, FCBBF3015317, FCBBF3015727, FCBBF3015758, FCBBF3015809, FCBBF3015870, FCBBF3016018, FCBBF3016134, FCBBF3016178, FCBBF3016451, FCBBF3016618, FCBBF3016622, FCBBF3016644, FCBBF3016667, FCBBF3016987, FCBBF3017024, FCBBF3017059, FCBBF3017071, FCBBF3017123, FCBBF3017233, FCBBF3017255, FCBBF3017288, FCBBF3017535, FCBBF3017681, FCBBF3017729, FCBBF3017873, FCBBF3017918, FCBBF3017974, FCBBF3018067, FCBBF3018173,

FCBBF3018453, FCBBF3018591, FCBBF3018753, FCBBF3018826, FCBBF3018949, FCBBF3019085, FCBBF3019320, FCBBF3019437, FCBBF3019455, FCBBF3019564, FCBBF3019569, FCBBF3019570, FCBBF3019663, FCBBF3019716, FCBBF3019784, FCBBF3019867, FCBBF3019961, FCBBF3020030, FCBBF3020138, FCBBF3020140, FCBBF3020163, FCBBF3020232, FCBBF3020599, FCBBF3021026, FCBBF3021191, FCBBF3021221, FCBBF3021501, FCBBF3021506, FCBBF3021524, FCBBF3021940, FCBBF3021985, FCBBF3022005, FCBBF3022291, FCBBF3022311, FCBBF3022504, FCBBF3022566, FCBBF3022593, FCBBF3022765, FCBBF3022767, FCBBF3022894, FCBBF3023061, FCBBF3023078, FCBBF3023368, FCBBF3023372, FCBBF3023443, FCBBF3023599, FCBBF3023704, FCBBF3023887, FCBBF3023895, FCBBF3024096,

FCBBF3024225, FCBBF3024266, FCBBF3024364, FCBBF3024793, FCBBF3024911, FCBBF3025098, FCBBF3025252, FCBBF3025280, FCBBF3025285, FCBBF3025417, FCBBF3025650, FCBBF3025674, FCBBF3025730, FCBBF3026021, FCBBF3026048, FCBBF3026251, FCBBF3026308, FCBBF3026651, FCBBF3026678, FCBBF3026692, FCBBF3027104, FCBBF3027199, FCBBF3027328, FCBBF3027559, FCBBF3027717, FCBBF3027755, FCBBF3027768, FCBBF3027854, FCBBF3027863, FCBBF3027903, FCBBF3028102, FCBBF3028143, FCBBF3028188, FCBBF3028202, FCBBF3028244, FCBBF3028472, FCBBF3028528, FCBBF3028582, FCBBF3028593, FCBBF3028671, FCBBF3028794, FCBBF4000017, FCBBF4000061, FCBBF4000076, FCBBF4000173, FCBBF4000192, FCBBF4000193, FCBBF4000268, FCBBF4000282, FCBBF4000399,

FCBBF4000415, FCBBF4000500, FCBBF4000529, FCBBF4000548, FCBBF5000041, FCBBF5000061, FCBBF5000165, FCBBF5000325, FCBBF5000353, FCBBF5000384, FCBBF5000483, FCBBF5000495, FEBRA1000005, FEBRA1000008, FEBRA1000030, FEBRA1000057, FEBRA1000138, FEBRA1000148, FEBRA1000149, FEBRA1000183, FEBRA1000188, FEBRA1000189, FEBRA1000190, FEBRA2000007, FEBRA2000021, FEBRA2000035, FEBRA2000053, FEBRA2000126, FEBRA2000220, FEBRA2000228, FEBRA2000253, FEBRA2000282, FEBRA2000286, FEBRA2000297, FEBRA2000311, FEBRA2000321, FEBRA2000333, FEBRA2000377, FEBRA2000378, FEBRA2000391, FEBRA2000394, FEBRA2000397, FEBRA2000399, FEBRA2000402, FEBRA2000404, FEBRA2000409, FEBRA2000415, FEBRA2000436, FEBRA2000438, FEBRA2000452,

FEBRA2000454, FEBRA2000468, FEBRA2000491, FEBRA2000504, FEBRA2000532, FEBRA2000538, FEBRA2000575, FEBRA2000656, FEBRA2000659, FEBRA2000690, FEBRA2000727, FEBRA2000734, FEBRA2000762, FEBRA2000771, FEBRA2000772, FEBRA2000782, FEBRA2000787, FEBRA2000790, FEBRA2000793, FEBRA2000803, FEBRA2000805, FEBRA2000809, FEBRA2000815, FEBRA2000862, FEBRA2000877, FEBRA2000881, FEBRA2000901, FEBRA2000909, FEBRA2000917, FEBRA2000928, FEBRA2000936, FEBRA2000937, FEBRA2000959, FEBRA2000972, FEBRA2001004, FEBRA2001012, FEBRA2001020, FEBRA2001093, FEBRA2001124, FEBRA2001133, FEBRA2001146, FEBRA2001161, FEBRA2001175, FEBRA2001197, FEBRA2001217, FEBRA2001227, FEBRA2001245, FEBRA2001267, FEBRA2001294, FEBRA2001334,

FEBRA2001351, FEBRA2001383, FEBRA2001438, FEBRA2001440, FEBRA2001487, FEBRA2001492, FEBRA2001523, FEBRA2001561, FEBRA2001571, FEBRA2001581, FEBRA2001584, FEBRA2001590, FEBRA2001591, FEBRA2001610, FEBRA2001620, FEBRA2001659, FEBRA2001669, FEBRA2001698, FEBRA2001705, FEBRA2001706, FEBRA2001715, FEBRA2001752, FEBRA2001772, FEBRA2001790, FEBRA2001814, FEBRA2001867, FEBRA2001914, FEBRA2001974, FEBRA2001989, FEBRA2001990, FEBRA2002009, FEBRA2002027, FEBRA2002086, FEBRA2002109, FEBRA2002191, FEBRA2002260, FEBRA2002352, FEBRA2002399, FEBRA2002410, FEBRA2002429, FEBRA2002442, FEBRA2002456, FEBRA2002508, FEBRA2002525, FEBRA2002552, FEBRA2002574, FEBRA2002601, FEBRA2002611, FEBRA2002628, FEBRA2002662,

FEBRA2002682, FEBRA2002707, FEBRA2002727, FEBRA2002781, FEBRA2002783, FEBRA2002792, FEBRA2002818, FEBRA2002858, FEBRA2002882, FEBRA2002897, FEBRA2002908, FEBRA2002931, FEBRA2002933, FEBRA2002962, FEBRA2002986, FEBRA2003054, FEBRA2003100, FEBRA2003115, FEBRA2003128, FEBRA2003155, FEBRA2003163, FEBRA2003181, FEBRA2003198, FEBRA2003253, FEBRA2003258, FEBRA2003275, FEBRA2003308, FEBRA2003327, FEBRA2003429, FEBRA2003454, FEBRA2003468, FEBRA2003470, FEBRA2003520, FEBRA2003524, FEBRA2003541, FEBRA2003675, FEBRA2003707, FEBRA2003711, FEBRA2003726, FEBRA2003822, FEBRA2003897, FEBRA2003907, FEBRA2003930, FEBRA2004023, FEBRA2004026, FEBRA2004029, FEBRA2004053, FEBRA2004056, FEBRA2004091, FEBRA2004110,

FEBRA2004191, FEBRA2004202, FEBRA2004219, FEBRA2004224, FEBRA2004237, FEBRA2004268, FEBRA2004293, FEBRA2004325, FEBRA2004329, FEBRA2004331, FEBRA2004443, FEBRA2004485, FEBRA2004490, FEBRA2004538, FEBRA2004592, FEBRA2004620, FEBRA2004628, FEBRA2004651, FEBRA2004767, FEBRA2004813, FEBRA2004818, FEBRA2004840, FEBRA2004867, FEBRA2004914, FEBRA2004933, FEBRA2004987, FEBRA2005014, FEBRA2005036, FEBRA2005079, FEBRA2005114, FEBRA2005216, FEBRA2005291, FEBRA2005361, FEBRA2005377, FEBRA2005380, FEBRA2005416, FEBRA2005427, FEBRA2005458, FEBRA2005476, FEBRA2005572, FEBRA2005701, FEBRA2005726, FEBRA2005734, FEBRA2005778, FEBRA2005787, FEBRA2005788, FEBRA2005824, FEBRA2005995, FEBRA2005998, FEBRA2006055,

FEBRA2006061, FEBRA2006112, FEBRA2006150, FEBRA2006165, FEBRA2006255, FEBRA2006270, FEBRA2006315, FEBRA2006346, FEBRA2006354, FEBRA2006357, FEBRA2006368, FEBRA2006370, FEBRA2006372, FEBRA2006379, FEBRA2006396, FEBRA2006401, FEBRA2006409, FEBRA2006418, FEBRA2006427, FEBRA2006476, FEBRA2006485, FEBRA2006491, FEBRA2006519, FEBRA2006521, FEBRA2006553, FEBRA2006627, FEBRA2006664, FEBRA2006667, FEBRA2006726, FEBRA2006736, FEBRA2006793, FEBRA2006808, FEBRA2006817, FEBRA2006873, FEBRA2006890, FEBRA2006942, FEBRA2006977, FEBRA2007017, FEBRA2007176, FEBRA2007200, FEBRA2007212, FEBRA2007247, FEBRA2007280, FEBRA2007414, FEBRA2007436, FEBRA2007449, FEBRA2007458, FEBRA2007534, FEBRA2007544, FEBRA2007551,

FEBRA2007566, FEBRA2007589, FEBRA2007613, FEBRA2007620, FEBRA2007622, FEBRA2007708, FEBRA2007793, FEBRA2007801, FEBRA2007823, FEBRA2007880, FEBRA2007900, FEBRA2007931, FEBRA2007957, FEBRA2008009, FEBRA2008036, FEBRA2008086, FEBRA2008134, FEBRA2008135, FEBRA2008159, FEBRA2008165, FEBRA2008172, FEBRA2008201, FEBRA2008265, FEBRA2008266, FEBRA2008282, FEBRA2008287, FEBRA2008311, FEBRA2008341, FEBRA2008360, FEBRA2008459, FEBRA2008468, FEBRA2008475, FEBRA2008481, FEBRA2008542, FEBRA2008660, FEBRA2008662, FEBRA2008681, FEBRA2008692, FEBRA2008741, FEBRA2008755, FEBRA2008836, FEBRA2008859, FEBRA2008861, FEBRA2008866, FEBRA2008881, FEBRA2008928, FEBRA2008983, FEBRA2009016, FEBRA2009022, FEBRA2009029,

FEBRA2009074, FEBRA2009117, FEBRA2009120, FEBRA2009162, FEBRA2009185, FEBRA2009240, FEBRA2009265, FEBRA2009276, FEBRA2009289, FEBRA2009327, FEBRA2009328, FEBRA2009352, FEBRA2009362, FEBRA2009385, FEBRA2009416, FEBRA2009437, FEBRA209478, FEBRA2009507, FEBRA2009514, FEBRA2009541, FEBRA2009567, FEBRA2009588, FEBRA2009731, FEBRA2009754, FEBRA2009780, FEBRA2009846, FEBRA2009986, FEBRA2010141, FEBRA2010299, FEBRA2010484, FEBRA2010719, FEBRA2010746, FEBRA2010768, FEBRA2010802, FEBRA2011063, FEBRA2011356, FEBRA2011414, FEBRA2011593, FEBRA2011665, FEBRA2012090, FEBRA2012120, FEBRA2012181, FEBRA2012195, FEBRA2012397, FEBRA2012418, FEBRA2012507, FEBRA2012625, FEBRA2013019, FEBRA2013069, FEBRA2013274,

FEBRA2013465, FEBRA2013570, FEBRA2013699, FEBRA2013905, FEBRA2013951, FEBRA2014010, FEBRA2014122, FEBRA2014198, FEBRA2014213, FEBRA2014297, FEBRA2014443, FEBRA2014578, FEBRA2014939, FEBRA2015076, FEBRA2015085, FEBRA2015175, FEBRA2015292, FEBRA2015588, FEBRA2016112, FEBRA2016398, FEBRA2016572, FEBRA2016583, FEBRA2016621, FEBRA2016654, FEBRA2016739, FEBRA2017024, FEBRA2017098, FEBRA2017138, FEBRA2017154, FEBRA2017200, FEBRA2017223, FEBRA2017333, FEBRA2017502, FEBRA2017533, FEBRA2017680, FEBRA2017736, FEBRA2017780, FEBRA2017811, FEBRA2017850, FEBRA2018051, FEBRA2018203, FEBRA2018407, FEBRA2018433, FEBRA2018476, FEBRA2018746, FEBRA2019172, FEBRA2019242, FEBRA2019298, FEBRA2019389, FEBRA2019457,

FEBRA2019519, FEBRA2019582, FEBRA2019637, FEBRA2019663, FEBRA2019690, FEBRA2019711, FEBRA2019867, FEBRA2020406, FEBRA2020566, FEBRA2020582, FEBRA2020668, FEBRA2020801, FEBRA2020845, FEBRA2020886, FEBRA2020977, FEBRA2021032, FEBRA2021134, FEBRA2021171, FEBRA2021339, FEBRA2021431, FEBRA2021497, FEBRA2021550, FEBRA2021571, FEBRA2021636, FEBRA2021864, FEBRA2021892, FEBRA2021908, FEBRA2021966, FEBRA2022013, FEBRA2022148, FEBRA2022204, FEBRA2022255, FEBRA2022322, FEBRA2022601, FEBRA2022696, FEBRA2022729, FEBRA2022787, FEBRA2022911, FEBRA2022956, FEBRA2022963, FEBRA2022972, FEBRA2023227, FEBRA2023285, FEBRA2023351, FEBRA2023377, FEBRA2023399, FEBRA2023498, FEBRA2023550, FEBRA2023721, FEBRA2023989,

FEBRA2023990, FEBRA2024019, FEBRA2024136, FEBRA2024150, FEBRA2024343, FEBRA2024570, FEBRA2024693, FEBRA2024727, FEBRA2024744, FEBRA2024797, FEBRA2024987, FEBRA2025249, FEBRA2025427, FEBRA2025463, FEBRA2025477, FEBRA2026474, FEBRA2026582, FEBRA2026629, FEBRA2026769, FEBRA2026879, FEBRA2026890, FEBRA2026977, FEBRA2027082, FEBRA2027297, FEBRA2027352, FEBRA2027364, FEBRA2027609, FEBRA2028158, FEBRA2028222, FEBRA2028366, FEBRA2028457, FEBRA2028477, FEBRA2028503, FEHRT2000325, FEHRT2000364, FEKID1000018, FELIV1000101, FELIV1000137, FELIV1000164, FELNG2000241, HCASM1000021, HCASM1000050, HCASM1000061, HCASM1000115, HCASM1000130, HCASM2000156, HCASM2000202, HCASM2000214, HCASM2000266, HCASM2000307,

HCASM2000534, HCASM2000536, HCASM2000560, HCASM2000871, HCASM2001285, HCASM2001810, HCASM2001866, HCASM2002148, HCASM2002502, HCASM2002754, HCASM2002918, HCASM2003018, HCASM2003076, HCASM2003099, HCASM2003212, HCASM2003237, HCASM2003357, HCASM2003415, HCASM2005388, HCASM2005484, HCASM2006131, HCASM2006338, HCASM2006359, HCASM2006632, HCASM2007047, HCASM2007317, HCASM2007737, HCASM2007773, HCASM2008154, HCASM2009405, HCASM2009424, HCASM2009463, HCASM2009588, HCHON1000015, HCHON1000166, HCHON1000176, HCHON1000191, HCHON2000028, HCHON2000038, HCHON2000062, HCHON2000100, HCHON2000156, HCHON2000199, HCHON2000212, HCHON2000271, HCHON2000315, HCHON2000322, HCHON2000344, HCHON2000364, HCHON2000373,

HCHON2000395, HCHON2000417, HCHON2000473, HCHON2000503, HCHON2000508, HCHON2000660, HCHON2000663, HCHON2000673, HCHON2000676, HCHON2000699, HCHON2000705, HCHON2000751, HCHON2000815, HCHON2000817, HCHON2000818, HCHON2000819, HCHON2000832, HCHON2000851, HCHON2000898, HCHON2000935, HCHON2000942, HCHON2000956, HCHON2000994, HCHON2001050, HCHON2001099, HCHON2001108, HCHON2001110, HCHON2001116, HCHON2001161, HCHON2001214, HCHON2001233, HCHON2001269, HCHON2001359, HCHON2001407, HCHON2001450, HCHON2001453, HCHON2001497, HCHON2001519, HCHON2001523, HCHON2001535, HCHON2001546, HCHON2001548, HCHON2001577, HCHON2001604, HCHON2001607, HCHON2001646, HCHON2001712, HCHON2001768, HCHON2001801, HCHON2002123,

HCHON2002194, HCHON2002241, HCHON2002354, HCHON2002496, HCHON2002676, HCHON2002770, HCHON2002964, HCHON2002971, HCHON2003131, HCHON2003134, HCHON2003327, HCHON2003513, HCHON2003532, HCHON2003642, HCHON2003659, HCHON2003676, HCHON2004007, HCHON2004279, HCHON2004359, HCHON2004531, HCHON2004776, HCHON2004858, HCHON2005048, HCHON2005118, HCHON2005166, HCHON2005802, HCHON2005987, HCHON2006459, HCHON2006714, HCHON2006722, HCHON2006770, HCHON2006786, HCHON2006841, HCHON2006871, HCHON2007046, HCHON2007482, HCHON2007674, HCHON2007774, HCHON2007881, HCHON2008112, HCHON2008239, HCHON2008582, HCHON2008672, HCHON2008856, HCHON2008871, HCHON2008989, HCHON2009191, HCHON2009384, HCHON2009749, HCHON2009766,

HCHON2009795, HCHON2010074, HCHON2010543, HEART1000010, HEART1000074, HEART1000088, HEART1000099, HEART1000102, HEART1000118, HEART1000132, HEART1000139, HEART1000142, HEART1000151, HEART1000173, HEART2000024, HEART2000035, HEART2000099, HEART2000298, HEART2000306, HEART2000309, HEART2000411, HEART2000418, HEART2000444, HEART2000448, HEART2000492, HEART2000520, HEART2000541, HEART2000568, HEART2000611, HEART2000959, HEART2001680, HEART2001756, HEART2002129, HEART2002184, HEART2002220, HEART2002432, HEART2002531, HEART2002598, HEART2002717, HEART2003027, HEART2003168, HEART2003432, HEART2003440, HEART2003781, HEART2003836, HEART2003852, HEART2004570, HEART2004764, HEART2004940, HEART2004941,

HEART2004980, HEART2005244, HEART2005310, HEART2005581, HEART2005610, HEART2006131, HEART2006195, HEART2006310, HEART2006334, HEART2006487, HEART2006521, HEART2006557, HEART2006787, HEART2006789, HEART2006909, HEART2007031, HEART2007443, HEART2007696, HEART2007767, HEART2008108, HEART2008111, HEART2008257, HEART2008509, HEART2008994, HEART2009000, HEART2009599, HEART2009680, HEART2010391, HEART2010492, HEART2010495, HELAC2000158, HELAC2000301, HHDPC1000001, HHDPC1000065, HHDPC1000083, HHDPC1000118, HHDPC2000055, HHDPC2000070, HHDPC2000102, HHDPC2000104, HHDPC2000115, HHDPC2000149, HHDPC2000258, HHDPC2000315, HHDPC2000456, HHDPC2000462, HHDPC2000692, HHDPC2001337, HHDPC2001432, HHDPC2001896,

HHDPC2002963, HHDPC2003049, HHDPC2003113, HHDPC2003381, HHDPC2003472, HHDPC2004757, HHDPC2005185, HHDPC2005513, HHDPC2005742, HHDPC2005794, HHDPC2006460, HHDPC2006862, HHDPC2006927, HHDPC2007302, HHDPC2008123, HHDPC2008185, HHDPC2008279, HHDPC2008297, HHDPC2008414, HHDPC2008474, HHDPC2008816, HHDPC2008843, HHDPC2009114, HHDPC2009178, HHDPC2009208, HHDPC2009272, HHDPC2009528, HLUNG1000017, HLUNG1000024, HLUNG1000030, HLUNG1000034, HLUNG1000037, HLUNG1000053, HLUNG1000084, HLUNG1000091, HLUNG1000099, HLUNG1000104, HLUNG1000105, HLUNG1000110, HLUNG1000151, HLUNG1000169, HLUNG2000014, HLUNG2000027, HLUNG2000068, HLUNG2000116, HLUNG2000125, HLUNG2000130, HLUNG2000142, HLUNG2000174, HLUNG2000176,

HLUNG2000217, HLUNG2000232, HLUNG2000255, HLUNG2000281, HLUNG2000315, HLUNG2000340, HLUNG2000362, HLUNG2000363, HLUNG2000431, HLUNG2000480, HLUNG2000489, HLUNG2000549, HLUNG2000557, HLUNG2000570, HLUNG2000751, HLUNG2000761, HLUNG2000777, HLUNG2000870, HLUNG2000926, HLUNG2000950, HLUNG2000955, HLUNG2000970, HLUNG2001013, HLUNG2001126, HLUNG2001144, HLUNG2001214, HLUNG2001439, HLUNG2001459, HLUNG2001633, HLUNG2001712, HLUNG2001814, HLUNG2001996, HLUNG2002006, HLUNG2002050, HLUNG2002059, HLUNG2002085, HLUNG2002145, HLUNG2002295, HLUNG2002303, HLUNG2002334, HLUNG2002405, HLUNG2002429, HLUNG2002465, HLUNG2002562, HLUNG2002659, HLUNG2002707, HLUNG2002916, HLUNG2002942, HLUNG2002949, HLUNG2002958,

HLUNG2002982, HLUNG2003042, HLUNG2003049, HLUNG2003061, HLUNG2003162, HLUNG2003246, HLUNG2003306, HLUNG2003331, HLUNG2003335, HLUNG2003378, HLUNG2003497, HLUNG2003691, HLUNG2003710, HLUNG2003714, HLUNG2003716, HLUNG2003778, HLUNG2003833, HLUNG2003872, HLUNG2003918, HLUNG2003991, HLUNG2004014, HLUNG2004037, HLUNG2004067, HLUNG2004154, HLUNG2004217, HLUNG2004273, HLUNG2004388, HLUNG2004416, HLUNG2004521, HLUNG2004534, HLUNG2004707, HLUNG2004774, HLUNG2004775, HLUNG2005012, HLUNG2005030, HLUNG2005045, HLUNG2005076, HLUNG2005133, HLUNG2005203, HLUNG2005230, HLUNG2005479, HLUNG2005524, HLUNG2005656, HLUNG2005738, HLUNG2005774, HLUNG2005813, HLUNG2005924, HLUNG2006026, HLUNG2006067, HLUNG2006094,

HLUNG2006370, HLUNG2006397, HLUNG2006570, HLUNG2006599, HLUNG2006614, HLUNG2006671, HLUNG2006781, HLUNG2006843, HLUNG2006852, HLUNG2006935, HLUNG2007041, HLUNG2007163, HLUNG2007210, HLUNG2007219, HLUNG2007245, HLUNG2007433, HLUNG2007931, HLUNG2008066, HLUNG2008139, HLUNG2008153, HLUNG2008225, HLUNG2008235, HLUNG2008247, HLUNG2008348, HLUNG2008384, HLUNG2008396, HLUNG2008439, HLUNG2008479, HLUNG2008480, HLUNG2008521, HLUNG2008833, HLUNG2008875, HLUNG2008885, HLUNG2008958, HLUNG2008959, HLUNG2009067, HLUNG2009215, HLUNG2009253, HLUNG2009303, HLUNG2009413, HLUNG2009729, HLUNG2010181, HLUNG2010464, HLUNG2010562, HLUNG2010845, HLUNG2011041, HLUNG2011298, HLUNG2011461, HLUNG2011833, HLUNG2012049,

HLUNG2012287, HLUNG2012315, HLUNG2012546, HLUNG2012600, HLUNG2012727, HLUNG2013097, HLUNG2013204, HLUNG2013304, HLUNG2013350, HLUNG2013622, HLUNG2013643, HLUNG2013784, HLUNG2013851, HLUNG2014262, HLUNG2014288, HLUNG2014449, HLUNG2014821, HLUNG2015418, HLUNG2015548, HLUNG2015578, HLUNG2015617, HLUNG2016022, HLUNG2016862, HLUNG2017286, HLUNG2017307, HLUNG2017350, HLUNG2017546, HLUNG2017806, HLUNG2018029, HLUNG2018282, HLUNG2018626, HLUNG2019058, HSYRA1000119, HSYRA1000131, HSYRA1000137, HSYRA1000148, HSYRA2000044, HSYRA2000135, HSYRA2000137, HSYRA2000190, HSYRA2000221, HSYRA2000237, HSYRA2000253, HSYRA2000332, HSYRA2000371, HSYRA2000424, HSYRA2000507, HSYRA2000510, HSYRA2000605, HSYRA2000629,

HSYRA2000706, HSYRA2000760, HSYRA2000787, HSYRA2000792, HSYRA2000828, HSYRA2000832, HSYRA2000925, HSYRA2000927, HSYRA2001003, HSYRA2000103, HSYRA2001142, HSYRA2001153, HSYRA2001225, HSYRA2001232, HSYRA2001255, HSYRA2001332, HSYRA2001353, HSYRA2001420, HSYRA2001484, HSYRA2001552, HSYRA2001595, HSYRA2001631, HSYRA2001638, HSYRA2001983, HSYRA2002103, HSYRA2002590, HSYRA2002741, HSYRA2003168, HSYRA2004550, HSYRA2004858, HSYRA2005456, HSYRA2005496, HSYRA2005628, HSYRA2005970, HSYRA2006049, HSYRA2006873, HSYRA2007241, HSYRA2007338, HSYRA2007667, HSYRA2008154, HSYRA2008714, HSYRA2009102, IMR321000155, IMR321000161, IMR321000165, IMR321000193, IMR321000207, IMR321000230, IMR321000253, IMR322000010,

IMR322000072, IMR322000107, IMR322000127, IMR322000144, IMR322000152, IMR322000164, IMR322000178, IMR322000223, IMR322000243, IMR322000302, IMR322000316, IMR322000374, IMR322000379, IMR322000383, IMR322000418, IMR322000518, IMR322000555, IMR322000604, IMR322000609, IMR322000656, IMR322000672, IMR322000742, IMR322000775, IMR322000791, IMR322000811, IMR322000819, IMR322000838, IMR322000859, IMR322000863, IMR322000917, IMR322000918, IMR322000919, IMR322000953, IMR322000973, IMR322000984, IMR322001018, IMR322001167, IMR322001185, IMR322001218, IMR322001245, IMR322001317, IMR322001318, IMR322001322, IMR322001332, IMR322001380, IMR322001438, IMR322001452, IMR322001491, IMR322001541, IMR322001584,

IMR322001665, IMR322001670, IMR322001724, IMR322002035, IMR322002110, IMR322002470, IMR322002993, IMR322003675, IMR322004768, IMR322005293, IMR322005833, IMR322006012, IMR322006222, IMR322006495, IMR322006520, IMR322006886, IMR322006947, IMR322007078, IMR322007225, IMR322007704, IMR322008651, IMR322009710, IMR322009807, IMR322010295, IMR322010953, IMR322011689, IMR322012314, IMR322012529, IMR322013053, IMR322013396, IMR322013731, IMR322013805, IMR322015523, IMR322016146, IMR322017049, IMR322018117, IMR322018192, JCMLC2000273, KIDNE1000008, KIDNE1000012, KIDNE1000015, KIDNE1000028, KIDNE1000064, KIDNE1000104, KIDNE1000121, KIDNE1000143, KIDNE1000152, KIDNE2000026, KIDNE2600041, KIDNE2000046,

KIDNE2000051, KIDNE2000070, KIDNE2000167, KIDNE2000174, KIDNE2000192, KIDNE2000227, KIDNE2000244, KIDNE2000245, KIDNE2000252, KIDNE2000266, KIDNE2000287, KIDNE2000315, KIDNE2000317, KIDNE2000328, KIDNE2000335, KIDNE2000349, KIDNE2000375, KIDNE2000383, KIDNE2000394, KIDNE2000422, KIDNE2000493, KIDNE2000497, KIDNE2000510, KIDNE2000513, KIDNE2000517, KIDNE2000519, KIDNE2000555, KIDNE2000574, KIDNE2000624, KIDNE2000665, KIDNE2000678, KIDNE2000717, KIDNE2000721, KIDNE2000722, KIDNE2000777, KIDNE2000801, KIDNE2000832, KIDNE2000833, KIDNE2000846, KIDNE2000867, KIDNE2000909, KIDNE2000947, KIDNE2001117, KIDNE2001140, KIDNE2001269, KIDNE2001361, KIDNE2001373, KIDNE2001636, KIDNE2001713, KIDNE2001802,

KIDNE2001840, KIDNE2001847, KIDNE2001857, KIDNE2001873, KIDNE2001897, KIDNE2001931, KIDNE2002015, KIDNE2002162, KIDNE2002168, KIDNE2002191, KIDNE2002198, KIDNE2002262, KIDNE2002265, KIDNE2002438, KIDNE2002483, KIDNE2002725, KIDNE2002768, KIDNE2002795, KIDNE2002798, KIDNE2002839, KIDNE2002845, KIDNE2002872, KIDNE2002882, KIDNE2002883, KIDNE2002980, KIDNE2002991, KIDNE2003185, KIDNE2003188, KIDNE2003233, KIDNE2003316, KIDNE2003335, KIDNE2003357, KIDNE2003373, KIDNE2003377, KIDNE2003507, KIDNE2003752, KIDNE2003764, KIDNE2003837, KIDNE2003994, KIDNE2004034, KIDNE2004054, KIDNE2004084, KIDNE2004095, KIDNE2004115, KIDNE2004233, KIDNE2004262, KIDNE2004279, KIDNE2004294, KIDNE2004295, KIDNE2004305,

KIDNE2004344, KIDNE2004394, KIDNE2004411, KIDNE2004475, KIDNE2004519, KIDNE2004520, KIDNE2004531, KIDNE2004534, KIDNE2004562, KIDNE2004579, KIDNE2004626, KIDNE2004681, KIDNE2004828, KIDNE2004846, KIDNE2004864, KIDNE2004879, KIDNE2004981, KIDNE2004985, KIDNE2004999, KIDNE2005038, KIDNE2005042, KIDNE2005062, KIDNE2005266, KIDNE2005296, KIDNE2005327, KIDNE2005336, KIDNE2005400, KIDNE2005424, KIDNE2005477, KIDNE2005526, KIDNE2005543, KIDNE2005603, KIDNE2005629, KIDNE2005781, KIDNE2005798, KIDNE2005854, KIDNE2005908, KIDNE2005937, KIDNE2006009, KIDNE2006039, KIDNE2006053, KIDNE2006062, KIDNE2006092, KIDNE2006145, KIDNE2006149, KIDNE2006210, KIDNE2006299, KIDNE2006334, KIDNE2006353, KIDNE2006376,

KIDNE2006378, KIDNE2006580, KIDNE2006652, KIDNE2006687, KIDNE2006733, KIDNE2006760, KIDNE2006775, KIDNE2006811, KIDNE2006820, KIDNE2006880, KIDNE2007005, KIDNE2007077, KIDNE2007186, KIDNE2007222, KIDNE2007356, KIDNE2007422, KIDNE2007569, KIDNE2007810, KIDNE2007910, KIDNE2007944, KIDNE2008022, KIDNE2008069, KIDNE2008072, KIDNE2008116, KIDNE2008117, KIDNE2008218, KIDNE2008315, KIDNE2008362, KIDNE2008378, KIDNE2008403, KIDNE2008404, KIDNE2008446, KIDNE2008473, KIDNE2008480, KIDNE2008649, KIDNE2008666, KIDNE2008697, KIDNE2008758, KIDNE2008788, KIDNE2008795, KIDNE2008824, KIDNE2008832, KIDNE2008869, KIDNE2008914, KIDNE2008975, KIDNE2008987, KIDNE2009019, KIDNE2009109, KIDNE2009180, KIDNE2009191,

KIDNE2009332, KIDNE2009426, KIDNE2009467, KIDNE2009490, KIDNE2009553, KIDNE2009628, KIDNE2009647, KIDNE2009676, KIDNE2009723, KIDNE2010007, KIDNE2010052, KIDNE2010084, KIDNE2010137, KIDNE2010151, KIDNE2010264, KIDNE2010265, KIDNE2010271, KIDNE2010419, KIDNE2010430, KIDNE2010574, KIDNE2010674, KIDNE2010739, KIDNE2010750, KIDNE2010762, KIDNE2010863, KIDNE2010973, KIDNE2010989, KIDNE2011200, KIDNE2011314, KIDNE2011508, KIDNE2011532, KIDNE2011635, KIDNE2011752, KIDNE2011782, KIDNE2011858, KIDNE2012009, KIDNE2012025, KIDNE2012188, KIDNE2012291, KIDNE2012316, KIDNE2012361, KIDNE2012440, KIDNE2012453, KIDNE2012563, KIDNE2012601, KIDNE2012613, KIDNE2012710, KIDNE2012745, KIDNE2012775, KIDNE2012945,

KIDNE2013045, KIDNE2013095, KIDNE2013218, KIDNE2013263, KIDNE2013346, KIDNE2013388, KIDNE2013413, KIDNE2013489, KIDNE2013639, KIDNE2013731, KIDNE2013734, KIDNE2013775, KIDNE2013801, KIDNE2013866, KIDNE2014087, KIDNE2014112, KIDNE2014119, KIDNE2014170, KIDNE2014184, KIDNE2014268, KIDNE2014298, KIDNE2014320, KIDNE2014415, KIDNE2014489, KIDNE2014496, KIDNE2014661, KIDNE2014717, KIDNE2014789, KIDNE2014808, KIDNE2014890, KIDNE2014978, KIDNE2015073, KIDNE2015221, KIDNE2015244, KIDNE2015313, KIDNE2015433, KIDNE2015445, KIDNE2015483, KIDNE2015515, KIDNE2015546, KIDNE2015556, KIDNE2015598, KIDNE2015660, KIDNE2015710, KIDNE2015987, KIDNE2016000, KIDNE2016036, KIDNE2016096, KIDNE2016101, KIDNE2016142,

KIDNE2016188, KIDNE2016327, KIDNE2016371, KIDNE2016388, KIDNE2016464, KIDNE2016539, KIDNE2016570, KIDNE2016918, KIDNE2017007, KIDNE2017052, KIDNE2017151, KIDNE2017153, KIDNE2017315, KIDNE2017332, KIDNE2017343, KIDNE2017454, KIDNE2017603, KIDNE2017956, KIDNE2018071, KIDNE2018166, KIDNE2018167, KIDNE2018217, KIDNE2018254, KIDNE2018268, KIDNE2018269, KIDNE2018287, KIDNE2018294, KIDNE2018352, KIDNE2018462, KIDNE2018493, KIDNE2018617, KIDNE2018678, KIDNE2018727, KIDNE2018863, KIDNE2018891, KIDNE2018989, KIDNE2018996, KIDNE2019074, KIDNE2019187, LIVER1000017, LIVER1000050, LIVER1000055, LIVER1000058, LIVER1000063, LIVER1000067, LIVER1000073, LIVER1000082, LIVER1000099, LIVER1000104, LIVER1000111,

LIVER1000126, LIVER1000139, LIVER1000155, LIVER1000165, LIVER1000175, LIVER1000224, LIVER1000230, LIVER1000278, LIVER1000303, LIVER1000397, LIVER1000421, LIVER1000433, LIVER1000479, LIVER1000482, LIVER1000542, LIVER2000003, LIVER2000037, LIVER2000158, LIVER2000216, LIVER2000237, LIVER2000247, LIVER2000360, LIVER2000416, LIVER2000446, LIVER2000515, LIVER2000592, LIVER2000626, LIVER2000682, LIVER2000769, LIVER2000775, LIVER2001051, LIVER2001076, LIVER2001099, LIVER2001113, LIVER2001164, LIVER2001191, LIVER2001265, LIVER2001329, LIVER2001389, LIVER2001461, LIVER2001835, LIVER2002638, LIVER2002644, LIVER2002660, LIVER2002702, LIVER2003008, LIVER2003065, LIVER2003511, LIVER2003524, LIVER2003568,

LIVER2003581, LIVER2003800, LIVER2003854, LIVER2004074, LIVER2004392, LIVER2004514, LIVER2004565, LIVER2005028, LIVER2005198, LIVER2005376, LIVER2005385, LIVER2005512, LIVER2005520, LIVER2005527, LIVER2005544, LIVER2005625, LIVER2005728, LIVER2005789, LIVER2005973, LIVER2005981, LIVER2006006, LIVER2006251, LIVER2006410, LIVER2006469, LIVER2006644, LIVER2006764, LIVER2007415, LIVER2007548, LIVER2007568, LIVER2007721, LIVER2008053, LIVER2008473, LIVER2008580, LIVER2008706, LIVER2008751, LIVER2008945, LIVER2008998, LIVER2009110, LIVER2009118, LIVER2009554, LYMPB1000141, LYMPB1000158, LYMPB2000083, MAMGL1000017, MAMGL1000032, MAMGL1000035, MAMGL1000096, MAMGL1000100, MAMGL1000132, MAMGL1000151,

MAMGL1000173, MAMGL1000178, MAMGL1000182, MAMGL1000184, MESAN1000032, MESAN1000035, MESAN1000050, MESAN1000079, MESAN1000080, MESAN1000101, MESAN1000121, MESAN1000126, MESAN1000147, MESAN1000180, MESAN2000023, MESAN2000026, MESAN2000067, MESAN2000092, MESAN2000149, MESAN2000167, MESAN2000291, MESAN2000337, MESAN2000434, MESAN2000457, MESAN2000462, MESAN2000501, MESAN2000572, MESAN2000608, MESAN2000620, MESAN2000711, MESAN2000815, MESAN2000909, MESAN2001450, MESAN2001627, MESAN2001770, MESAN2001979, MESAN2001980, MESAN2002113, MESAN2002122, MESAN2002147, MESAN2002186, MESAN2002519, MESAN2002687, MESAN2002709, MESAN2002790, MESAN2002940, MESAN2002978, MESAN2003035, MESAN2003037, MESAN2003101,

MESAN2003190, MESAN2003322, MESAN2003490, MESAN2003529, MESAN2003622, MESAN2003646, MESAN2003662, MESAN2003851, MESAN2004138, MESAN2004288, MESAN2004575, MESAN2005284, MESAN2005371, MESAN2005633, MESAN2005689, MESAN2005724, MESAN2005766, MESAN2005808, MESAN2005811, MESAN2005958, MESAN2006022, MESAN2006328, MESAN2006401, MESAN2006563, MESAN2006599, MESAN2006953, MESAN2006971, MESAN2007032, MESAN2007440, MESAN2007597, MESAN2007755, MESAN2008222, MESAN2008334, MESAN2008415, MESAN2008460, MESAN2008536, MESAN2008679, MESAN2008729, MESAN2008821, MESAN2008926, MESAN2008936, MESAN2009019, MESAN2009081, MESAN2009105, MESAN2009311, MESAN2009418, MESAN2009503, MESAN2009522, MESAN2010031, MESAN2010114,

MESAN2010312, MESAN2010321, MESAN2010664, MESAN2010681, MESAN2011545, MESAN2011597, MESAN2011627, MESAN2011632, MESAN2011977, MESAN2011984, MESAN2012054, MESAN2012113, MESAN2012586, MESAN2012708, MESAN2012735, MESAN2013019, MESAN2013022, MESAN2013183, MESAN2013211, MESAN2013283, MESAN2013611, MESAN2013674, MESAN2013845, MESAN2013908, MESAN2013936, MESAN2014192, MESAN2014298, MESAN2014412, MESAN2014624, MESAN2015277, MESAN2015365, MESAN2015391, MESAN2015401, MESAN2015501, MESAN2015515, MESAN2015708, MESAN2016159, MESAN2016304, MESAN2016409, MESAN2016965, MESAN2017133, MESAN2017373, MESAN2017417, MESAN2018209, MESAN2018576, MESAN2018670, MESAN2018699, MESTC1000042, MESTC1000147, MESTC2000150,

MESTC2000153, MESTC2000170, N1ESE2000698, NB9N41000011, NB9N41000047, NB9N41000135, NB9N41000142, NB9N41000146, NB9N41000340, NB9N42000104, NB9N42000122, NB9N42000196, NB9N42000314, NB9N42000342, NB9N42000430, NB9N42000495, NB9N42001300, NCRRP1000129, NESOP1000010, NESOP1000087, NESOP1000108, NESOP2000231, NESOP2000452, NESOP2000499, NESOP2000504, NESOP2000744, NESOP2001144, NESOP2001433, NESOP2001656, NESOP2001694, NESOP2001752, NESOP2002352, NESOP2002418, NESOP2002487, NESOP2002738, NESOP2002780, NETRP1000034, NETRP1000082, NETRP2000086, NETRP2000182, NETRP2000439, NHNPC1000034, NHNPC1000037, NHNPC1000084, NHNPC1000101, NHNPC1000124, NHNPC2000062, NHNPC2000187, NHNPC2000206, NHNPC2000212,

NHNPC2000606, NHNPC2000877, NHNPC2001108, NHNPC2001223, NHNPC2001312, NHNPC2001816, NHNPC2001931, NHNPC2002565, NHNPC2002749, NOVAR1000102, NOVAR2000038, NOVAR2000136, NOVAR2000352, NOVAR2000412, NOVAR2000783, NOVAR2000962, NOVAR2001108, NOVAR2001620, NOVAR2001783, NOVAR2001876, NOVAR2002039, NT2NE1000004, NT2NE1000014, NT2NE1000018, NT2NE1000023, NT2NE1000024, NT2NE1000059, NT2NE1000063, NT2NE1000073, NT2NE1000083, NT2NE1000120, NT2NE1000122, NT2NE1000175, NT2NE1000181, NT2NE1000185, NT2NE2000038, NT2NE2000054, NT2NE2000057, NT2NE2000064, NT2NE2000109, NT2NE2000137, NT2NE2000156, NT2NE2000174, NT2NE2000195, NT2NE2000222, NT2NE2000259, NT2NE2000260, NT2NE2000284, NT2NE2000299, NT2NE2000353,

NT2NE2000356, NT2NE2000369, NT2NE2000374, NT2NE2000386, NT2NE2000390, NT2NE2000392, NT2NE2000470, NT2NE2000488, NT2NE2000517, NT2NE2000536, NT2NE2000546, NT2NE2000548, NT2NE2000550, NT2NE2000586, NT2NE2000612, NT2NE2000636, NT2NE2000658, NT2NE2000706, NT2NE2000763, NT2NE2000802, NT2NE2000808, NT2NE2000809, NT2NE2000864, NT2NE2000909, NT2NE2000913, NT2NE2000950, NT2NE2000951, NT2NE2000980, NT2NE2001005, NT2NE2001021, NT2NE2001040, NT2NE2001048, NT2NE2001049, NT2NE2001142, NT2NE2001156, NT2NE2001180, NT2NE2001181, NT2NE2001188, NT2NE2001191, NT2NE2001247, NT2NE2001252, NT2NE2001324, NT2NE2001326, NT2NE2001364, NT2NE2001372, NT2NE2001397, NT2NE2001403, NT2NE2001428, NT2NE2001432, NT2NE2001442,

NT2NE2001524, NT2NE2001530, NT2NE2001598, NT2NE2001623, NT2NE2001626, NT2NE2001634, NT2NE2001648, NT2NE2001655, NT2NE2001666, NT2NE2001677, NT2NE2001697, NT2NE2001698, NT2NE2001723, NT2NE2001725, NT2NE2001874, NT2NE2001889, NT2NE2001923, NT2NE2002100, NT2NE2002162, NT2NE2002186, NT2NE2002285, NT2NE2002311, NT2NE2002651, NT2NE2002732, NT2NE2002768, NT2NE2002791, NT2NE2002856, NT2NE2002870, NT2NE2003150, NT2NE2003252, NT2NE2003280, NT2NE2003308, NT2NE2003309, NT2NE2003315, NT2NE2003393, NT2NE2003408, NT2NE2003457, NT2NE2003540, NT2NE2003571, NT2NE2003705, NT2NE2003752, NT2NE2003887, NT2NE2003921, NT2NE2004255, NT2NE2004378, NT2NE2004490, NT2NE2004606, NT2NE2004740, NT2NE2004787, NT2NE2004916,

NT2NE2005035, NT2NE2005223, NT2NE2005276, NT2NE2005317, NT2NE2005323, NT2NE2005371, NT2NE2005395, NT2NE2005419, NT2NE2005441, NT2NE2005517, NT2NE2005537, NT2NE2005555, NT2NE2005588, NT2NE2005676, NT2NE2005688, NT2NE2005720, NT2NE2005784, NT2NE2005804, NT2NE2005821, NT2NE2005876, NT2NE2005890, NT2NE2005921, NT2NE2005968, NT2NE2006075, NT2NE2006087, NT2NE2006103, NT2NE2006217, NT2NE2006288, NT2NE2006382, NT2NE2006387, NT2NE2006427, NT2NE2006432, NT2NE2006478, NT2NE2006531, NT2NE2006784, NT2NE2006813, NT2NE2006894, NT2NE2006909, NT2NE2006942, NT2NE2006958, NT2NE2007052, NT2NE2007220, NT2NE2007425, NT2NE2007725, NT2NE2007727, NT2NE2007786, NT2NE2007877, NT2NE2007967, NT2NE2008017, NT2NE2008060,

NT2NE2008077, NT2NE2008213, NT2NE2008260, NT2NE2008378, NT2NE2008607, NT2NE2008639, NT2NE2008727, NT2NE2008783, NT2NE2008785, NT2NE2008803, NT2NE2008867, NT2NE2008961, NT2NE2008997, NT2NE2009138, NT2NE2009426, NT2NE2009523, NT2NE2009608, NT2NE2009691, NT2NE2010056, NT2NE2010105, NT2NE2010150, NT2NE2010400, NT2NE2010632, NT2NE2010633, NT2NE2010654, NT2NE2010688, NT2NE2010781, NT2NE2010842, NT2NE2010854, NT2NE2011033, NT2NE2011036, NT2NE2011107, NT2NE2011119, NT2NE2011154, NT2NE2011221, NT2NE2011411, NT2NE2011485, NT2NE2011650, NT2NE2011684, NT2NE2.011687, NT2NE2011691, NT2NE2011758, NT2NE2011823, NT2NE2011896, NT2NE2011998, NT2NE2012113, NT2NE2012199, NT2NE2012243, NT2NE2012361, NT2NE2012448,

NT2NE2012457, NT2NE2012493, NT2NE2012505, NT2NE2012533, NT2NE2012603, NT2NE2012747, NT2NE2012790, NT2NE2012847, NT2NE2012928, NT2NE2013019, NT2NE2013081, NT2NE2013189, NT2NE2013217, NT2NE2013547, NT2NE2014013, NT2NE2014028, NT2NE2014104, NT2NE2014113, NT2NE2014176, NT2NE2014221, NT2NE2014525, NT2NE2014650, NT2NE2014651, NT2NE2014681, NT2NE2014758, NT2NE2014869, NT2NE2014950, NT2NE2015061, NT2NE2015262, NT2NE2015275, NT2NE2015362, NT2NE2015480, NT2NE2015511, NT2NE2015565, NT2NE2015626, NT2NE2015747, NT2NE2015860, NT2NE2015885, NT2NE2015904, NT2NE2016041, NT2NE2016387, NT2NE2016419, NT2NE2016519, NT2NE2016608, NT2NE2016766, NT2NE2016774, NT2NE2016971, NT2NE2017259, NT2NE2017332, NT2NE2017397,

NT2NE2017480, NT2NE2017508, NT2NE2017562, NT2NE2017590, NT2NE2017612, NT2NE2017721, NT2NE2017752, NT2NE2017792, NT2NE2017982, NT2NE2018165, NT2NE2018176, NT2NE2018180, NT2NE2018273, NT2NE2018376, NT2NE2018472, NT2NE2018490, NT2NE2018594, NT2NE2018739, NT2NE2018916, NT2NE2019056, NT2NE2019076, NT2NE2019091, NT2NE2019099, NT2RI1000016, NT2RI1000027, NT2RI1000035, NT2RI1000048, NT2RI1000164, NT2RI1000172, NT2RI2000004, NT2RI2000107, NT2RI2000116, NT2RI2000117, NT2RI2000128, NT2RI2000133, NT2RI2000167, NT2RI2000173, NT2RI2000187, NT2RI2000255, NT2RI2000263, NT2RI2000270, NT2RI2000276, NT2RI2000284, NT2RI2000313, NT2RI2000341, NT2RI2000348, NT2RI2000412, NT2RI2000421, NT2RI2000480, NT2RI2000575,

NT2RI2000588, NT2RI2000597, NT2RI2000669, NT2RI2000688, NT2RI2000704, NT2RI2000738, NT2RI2000775, NT2RI2000822, NT2RI2000829, NT2RI2000841, NT2RI2000865, NT2RI2000932, NT2RI2000974, NT2RI2000987, NT2RI2001010, NT2RI2001017, NT2RI2001030, NT2RI2001054, NT2RI2001083, NT2RI2001167, NT2RI2001178, NT2RI2001235, NT2RI2001244, NT2RI2001307, NT2RI2001342, NT2RI2001385, NT2RI2001409, NT2RI2001410, NT2RI2001422, NT2RI2001425, NT2RI2001449, NT2RI2001450, NT2RI2001474, NT2RI2001519, NT2RI2001595, NT2RI2001621, NT2RI2001624, NT2RI2001726, NT2RI2001731, NT2RI2001769, NT2RI2001782, NT2RI2001836, NT2RI2001846, NT2RI2001866, NT2RI2001910, NT2RI2001952, NT2RI2002022, NT2RI2002041, NT2RI2002117, NT2RI2002120,

NT2RI2002153, NT2RI2002179, NT2RI2002243, NT2RI2002252, NT2RI2002260, NT2RI2002266, NT2RI2002270, NT2RI2002334, NT2RI2002359, NT2RI2002447, NT2RI2002481, NT2RI2002507, NT2RI2002517, NT2RI2002530, NT2RI2002540, NT2RI2002549, NT2RI2002554, NT2RI2002564, NT2RI2002585, NT2RI2002592, NT2RI2002602, NT2RI2002699, NT2RI2002729, NT2RI2002802, NT2RI2002819, NT2RI2002822, NT2RI2002847, NT2RI2002852, NT2RI2002923, NT2RI2002926, NT2RI2002958, NT2RI2002970, NT2RI2003011, NT2RI2003019, NT2RI2003051, NT2RI2003067, NT2RI2003184, NT2RI2003205, NT2RI2003222, NT2RI2003301, NT2RI2003360, NT2RI2003361, NT2RI2003383, NT2RI2003392, NT2RI2003399, NT2RI2003419, NT2RI2003481, NT2RI2003487, NT2RI2003655, NT2RI2003667,

NT2RI2003678, NT2RI2003884, NT2RI2003921, NT2RI2003973, NT2RI2003993, NT2RI2004059, NT2RI2004078, NT2RI2004079, NT2RI2004093, NT2RI2004136, NT2RI2004190, NT2RI2004209, NT2RI2004284, NT2RI2004290, NT2RI2004304, NT2RI2004312, NT2RI2004381, NT2RI2004398, NT2RI2004410, NT2RI2004442, NT2RI2004468, NT2RI2004606, NT2RI2004608, NT2RI2004618, NT2RI2004783, NT2RI2004818, NT2RI2004840, NT2RI2004884, NT2RI2004916, NT2RI2004962, NT2RI2004984, NT2RI2004985, NT2RI2005048, NT2RI2005061, NT2RI2005069, NT2RI2005087, NT2RI2005096, NT2RI2005115, NT2RI2005130, NT2RI2005150, NT2RI2005166, NT2RI2005239, NT2RI2005246, NT2RI2005315, NT2RI2005353, NT2RI2005368, NT2RI2005382, NT2RI2005473, NT2RI2005520, NT2RI2005564,

NT2RI2005579, NT2RI2005621, NT2RI2005628, NT2RI2005647, NT2RI2005670, NT2RI2005710, NT2RI2005772, NT2RI2005816, NT2RI2005851, NT2RI2005966, NT2RI2006070, NT2RI2006071, NT2RI2006072, NT2RI2006081, NT2RI2006126, NT2RI2006127, NT2RI2006183, NT2RI2006257, NT2RI2006271, NT2RI2006294, NT2RI2006345, NT2RI2006403, NT2RI2006445, NT2RI2006487, NT2RI2006506, NT2RI2006553, NT2RI2006565, NT2RI2006640, NT2RI2006647, NT2RI2006659, NT2RI2006662, NT2RI2006667, NT2RI2006679, NT2RI2006682, NT2RI2006686, NT2RI2006703, NT2RI2006716, NT2RI2006735, NT2RI2006762, NT2RI2006788, NT2RI2006838, NT2RI2006853, NT2RI2006855, NT2RI2006856, NT2RI20069.43, NT2RI2006973, NT2RI2007046, NT2RI2007048, NT2RI2007084, NT2RI2007096,

NT2RI2007116, NT2RI2007133, NT2RI2007214, NT2RI2007303, NT2RI2007334, NT2RI2007377, NT2RI2007386, NT2RI2007406, NT2RI2007439, NT2RI2007468, NT2RI2007469, NT2RI2007507, NT2RI2007572, NT2RI2007593, NT2RI2007723, NT2RI2007729, NT2RI2007751, NT2RI2007754, NT2RI2007827, NT2RI2007853, NT2RI2007877, NT2RI2007879, NT2RI2007884, NT2RI2007891, NT2RI2007956, NT2RI2007965, NT2RI2008050, NT2RI2008141, NT2RI2008171, NT2RI2008188, NT2RI2008204, NT2RI2008221, NT2RI2008233, NT2RI2008396, NT2RI2008455, NT2RI2008502, NT2RI2008566, NT2RI2008598, NT2RI2008622, NT2RI2008656, NT2RI2008714, NT2RI2008801, NT2RI2008808, NT2RI2008812, NT2RI2008841, NT2RI2008952, NT2RI2009005, NT2RI2009066, NT2RI2009083, NT2RI2009101,

NT2RI2009103, NT2RI2009144, NT2RI2009151, NT2RI2009173, NT2RI2009194, NT2RI2009215, NT2RI2009233, NT2RI2009239, NT2RI2009259, NT2RI2009281, NT2RI2009289, NT2RI2009301, NT2RI2009517, NT2RI2009585, NT2RI2009595, NT2RI2009855, NT2RI2010283, NT2RI2010508, NT2RI2010795, NT2RI2011422, NT2RI2011450, NT2RI2011683, NT2RI2011803, NT2RI2012350, NT2RI2012659, NT2RI2012726, NT2RI2012990, NT2RI2013345, NT2RI2013357, NT2RI2013373, NT2RI2014551, NT2RI2014683, NT2RI2015239, NT2RI2015342, NT2RI2015533, NT2RI2016128, NT2RI2016134, NT2RI2018311, NT2RI2018363, NT2RI2018448, NT2RI2018883, NT2RI2018950, NT2RI2019751, NT2RI2019826, NT2RI2020390, NT2RI2020703, NT2RI2021463, NT2RI2021625, NT2RI2022584, NT2RI2023303,

NT2RI2023671, NT2RI2024008, NT2RI2024460, NT2RI2024496, NT2RI2024935, NT2RI2025075, NT2RI2025255, NT2RI2025564, NT2RI2025565, NT2RI2025909, NT2RI2025957, NT2RI2025976, NT2RI2026758, NT2RI2027081, NT2RI2027157, NT2RI2027224, NT2RI2027396, NT2RI2027484, NT2RI2027975, NT2RI2028213, NT2RI2028222, NT2RI2028537, NT2RI2028642, NT2RI3000174, NT2RI3000213, NT2RI3000423, NT2RI3000622, NT2RI3001132, NT2RI3001263, NT2RI3001279, NT2RI3001348, NT2RI3001458, NT2RI3001515, NT2RI3001573, NT2RI3001967, NT2RI3002303, NT2RI3002557, NT2RI3002842, NT2RI3003027, NT2RI3003031, NT2RI3003095, NT2RI3003104, NT2RI3003162, NT2RI3003362, NT2RI3003382, NT2RI3003409, NT2RI3003738, NT2RI3003925, NT2RI3004133, NT2RI3004381,

NT2RI3004510, NT2RI3005202, NT2RI3005403, NT2RI3005416, NT2RI3005861, NT2RI3005928, NT2RI3006132, NT2RI3006171, NT2RI3006376, NT2RI3006796, NT2RI3007065, NT2RI3007095, NT2RI3007158, NT2RI3007167, NT2RI3007213, NT2RI3007443, NT2RI3007684, NT2RI3007757, NT2RI3007878, NT2RI3007978, NT2RI3008055, NT2RI3008162, NT2RI3008179, NT2RI3008228, NT2RI3008442, NT2RI3008652, NT2RI3008697, NT2RI3008974, NT2RI3009158, NT2RI3009480, NT2RI3009524, NT2RP6000005, NT2RP6000008, NT2RP6000032, NT2RP6000033, NT2RP6000043, NT2RP6000060, NT2RP6000072, NT2RP6000086, NT2RP6000100, NT2RP6000109, NT2RP6000110, NT2RP6000119, NT2RP6000127, NT2RP6000130, NT2RP6000140, NT2RP6000150, NT2RP7000041, NT2RP7000086, NT2RP7000109,

NT2RP7000112, NT2RP7000238, NT2RP7000259, NT2RP7000271, NT2RP7000311, NT2RP7000359, NT2RP7000364, NT2RP7000380, NT2RP7000391, NT2RP7000425, NT2RP7000579, NT2RP7000624, NT2RP7000812, NT2RP7000926, NT2RP7001074, NT2RP7001080, NT2RP7001156, NT2RP7001231, NT2RP7001283, NT2RP7001306, NT2RP7001319, NT2RP7001335, NT2RP7001364, NT2RP7001475, NT2RP7001532, NT2RP7001591, NT2RP7001602, NT2RP7001678, NT2RP7001856, NT2RP7002064, NT2RP7002151, NT2RP7002163, NT2RP7002243, NT2RP7002282, NT2RP7002450, NT2RP7002478, NT2RP7002554, NT2RP7002594, NT2RP7002603, NT2RP7002619, NT2RP7002650, NT2RP7002738, NT2RP7002779, NT2RP7002829, NT2RP7002841, NT2RP7002906, NT2RP7002978, NT2RP7003025, NT2RP7003050, NT2RP7003084,

NT2RP7003091, NT2RP7003107, NT2RP7003134, NT2RP7003148, NT2RP7003261, NT2RP7003439, NT2RP7003511, NT2RP7003629, NT2RP7003680, NT2RP7003724, NT2RP7003960, NT2RP7004027, NT2RP7004080, NT2RP7004173, NT2RP7004204, NT2RP7004233, NT2RP7004260, NT2RP7004358, NT2RP7004396, NT2RP7004428, NT2RP7004481, NT2RP7004541, NT2RP7004641, NT2RP7004656, NT2RP7004687, NT2RP7004722, NT2RP7004766, NT2RP7004790, NT2RP7004884, NT2RP7004911, NT2RP7004961, NT2RP7004964, NT2RP7004975, NT2RP7005118, NT2RP7005205, NT2RP7005219, NT2RP7005323, NT2RP7005468, NT2RP7005493, NT2RP7005629, NT2RP7005631, NT2RP7005669, NT2RP7005846, NT2RP7006033, NT2RP7006075, NT2RP7006141, NT2RP7006160, NT2RP7006162, NT2RP7006188, NT2RP7006263,

NT2RP7006296, NT2RP7006304, NT2RP7006374, NT2RP7006395, NT2RP7006408, NT2RP7006457, NT2RP7006547, NT2RP7006601, NT2RP7006621, NT2RP7006636, NT2RP7006701, NT2RP7006853, NT2RP7006887, NT2RP7006980, NT2RP7006986, NT2RP7006995, NT2RP7007177, NT2RP7007221, NT2RP7007226, NT2RP7007269, NT2RP7007359, NT2RP7007387, NT2RP7007406, NT2RP7007480, NT2RP7007504, NT2RP7007530, NT2RP7007580, NT2RP7007594, NT2RP7007617, NT2RP7007643, NT2RP7007766, NT2RP7007842, NT2RP7007925, NT2RP7007930, NT2RP7007975, NT2RP7008013, NT2RP7008015, NT2RP7008133, NT2RP7008137, NT2RP7008142, NT2RP7008144, NT2RP7008360, NT2RP7008396, NT2RP7008406, NT2RP7008435, NT2RP7008441, NT2RP7008454, NT2RP7008487, NT2RP7008544, NT2RP7008550,

NT2RP7008557, NT2RP7008623, NT2RP7008657, NT2RP7008714, NT2RP7008720, NT2RP7008855, NT2RP7008863, NT2RP7009012, NT2RP7009019, NT2RP7009030, NT2RP7009108, NT2RP7009149, NT2RP7009168, NT2RP7009215, NT2RP7009225, NT2RP7009236, NT2RP7009253, NT2RP7009267, NT2RP7009322, NT2RP7009363, NT2RP7009370, NT2RP7009373, NT2RP7009394, NT2RP7009397, NT2RP7009429, NT2RP7009439, NT2RP7009466, NT2RP7009481, NT2RP7009498, NT2RP7009502, NT2RP7009507, NT2RP7009577, NT2RP7009743, NT2RP7009867, NT2RP7010109, NT2RP7010128, NT2RP7010235, NT2RP7010275, NT2RP7010521, NT2RP7010599, NT2RP7010817, NT2RP7011086, NT2RP7011132, NT2RP7011268, NT2RP7011318, NT2RP7011452, NT2RP7011570, NT2RP7011721, NT2RP7011909, NT2RP7012291,

NT2RP7012516, NT2RP7012776, NT2RP7013002, NT2RP7013374, NT2RP7013499, NT2RP7013729, NT2RP7013764, NT2RP7013795, NT2RP7013999, NT2RP7014005, NT2RP7014178, NT2RP7014348, NT2RP7014420, NT2RP7014583, NT2RP7014721, NT2RP7014743, NT2RP7014778, NT2RP7014906, NT2RP7014910, NT2RP7015080, NT2RP7015431, NT2RP7015503, NT2RP7015512, NT2RP7015676, NT2RP7015789, NT2RP7015996, NT2RP7016508, NT2RP7016574, NT2RP7016622, NT2RP7016911, NT2RP7017139, NT2RP7017284, NT2RP7017365, NT2RP7017546, NT2RP7017567, NT2RP7017795, NT2RP7017971, NT2RP7018032, NT2RP7018126, NT2RP7018197, NT2RP7018206, NT2RP7018340, NT2RP7018586, NT2RP7018802, NT2RP7018871, NT2RP7019064, NT2RP7019135, NT2RP7019273, NT2RP7019367, NT2RP7019401,

NT2RP7019445, NT2RP7019543, NT2RP7019682, NT2RP7019835, NT2RP7020112, NT2RP7020123, NT2RP7020343, NT2RP7020379, NT2RP8000137, NT2RP8000296, NT2RP8000435, NT2RP8000483, NT2RP8000521, NT2RP8000633, NT2RP8001363, NT2RP8001407, NT2RP8001584, NT2RP8001604, NT2RP8001605, NT2RP8003490, NT2RP8003657, NT2RP8003787, NT2RP8004306, NT2RP8005546, NT2RP8006452, NT2RP8006521, NT2RP8007416, NT2RP8007503, NT2RP8007715, NT2RP8007920, NT2RP8008057, NT2RP8009248, NTONG1000006, NTONG1000047, NTONG1000052, NTONG1000075, NTONG1000087, NTONG1000123, NTONG1000130, NTONG1000161, NTONG1000172, NTONG1000213, NTONG1000214, NTONG1000247, NTONG1000257, NTONG1000264, NTONG2000040, NTONG2000107, NTONG2000231, NTONG2000334,

NTONG2000363, NTONG2000413, NTONG2000492, NTONG2000499, NTONG2000531, NTONG2000583, NTONG2000800, NTONG2000858, NTONG2000878, NTONG2000966, NTONG2000977, NTONG2000985, NTONG2001014, NTONG2001079, NTONG2001137, NTONG2001157, NTONG2001173, NTONG2001220, NTONG2001222, NTONG2001362, NTONG2001428, NTONG2001532, NTONG2001587, NTONG2001612, NTONG2001619, NTONG2001642, NTONG2001657, NTONG2001676, NTONG2001762, NTONG2002278, NTONG2002807, NTONG2002948, NTONG2002970, NTONG2002985, NTONG2003158, NTONG2003210, NTONG2003316, NTONG2003409, NTONG2003515, NTONG2003805, NTONG2003852, NTONG2003897, NTONG2003928, NTONG2004095, NTONG2004308, NTONG2004521, NTONG2004614, NTONG2004690, NTONG2004806, NTONG2004829,

NTONG2004918, NTONG2004991, NTONG2005062, NTONG2005086, NTONG2005137, NTONG2005153, NTONG2005265, NTONG2005277, NTONG2005373, NTONG2005480, NTONG2005497, NTONG2005577, NTONG2005657, NTONG2005822, NTONG2005969, NTONG2006099, NTONG2006187, NTONG2006301, NTONG2006324, NTONG2006354, NTONG2006440, NTONG2006484, NTONG2006501, NTONG2006646, NTONG2006709, NTONG2006783, NTONG2007020, NTONG2007028, NTONG2007034, NTONG2007249, NTONG2007517, NTONG2007522, NTONG2007658, NTONG2007693, NTONG2007756, NTONG2008088, NTONG2008093, NTONG2008143, NTONG2008365, NTONG2008483, NTONG2008522, NTONG2008672, NTONG2008862, NTONG2008939, NTONG2009060, NTONG2009068, NTONG2009229, NTONG2009233, NTONG2009468, NTONG2009576,

OCBBF1000016, OCBBF1000030, OCBBF1000042, OCBBF1000049, OCBBF1000054, OCBBF1000067, OCBBF1000080, OCBBF1000085, OCBBF1000091, OCBBF1000104, OCBBF1000114, OCBBF1000118, OCBBF1000119, OCBBF1000122, OCBBF1000130, OCBBF1000138, OCBBF1000145, OCBBF1000146, OCBBF1000153, OCBBF1000185, OCBBF1000188, OCBBF1000192, OCBBF1000254, OCBBF1000315, OCBBF2000013, OCBBF2000126, OCBBF2000178, OCBBF2000231, OCBBF2000277, OCBBF2000287, OCBBF2000323, OCBBF2000452, OCBBF2000522, OCBBF2000703, OCBBF2000712, OCBBF2000719, OCBBF2000824, OCBBF2000831, OCBBF2000846, OCBBF2000904, OCBBF2000982, OCBBF2000998, OCBBF2001075, OCBBF2001101, OCBBF2001124, OCBBF2001140, OCBBF2001176, OCBBF2001186, OCBBF2001196, OCBBF2001252,

OCBBF2001307, OCBBF2001389, OCBBF2001402, OCBBF2001408, OCBBF2001416, OCBBF2001473, OCBBF2001492, OCBBF2001494, OCBBF2001527, OCBBF2001528, OCBBF2001639, OCBBF2001681, OCBBF2001687, OCBBF2001706, OCBBF2001749, OCBBF2001794, OCBBF2001910, OCBBF2001931, OCBBF2001983, OCBBF2002015, OCBBF2002083, OCBBF2002124, OCBBF2002161, OCBBF2002290, OCBBF2002357, OCBBF2002376, OCBBF2002615, OCBBF2002626, OCBBF2002663, OCBBF2002805, OCBBF2002865, OCBBF2002920, OCBBF2002980, OCBBF2003028, OCBBF2003052, OCBBF2003091, OCBBF2003327, OCBBF2003518, OCBBF2003543, OCBBF2003593, OCBBF2003744, OCBBF2003819, OCBBF2004038, OCBBF2004104, OCBBF2004147, OCBBF2004168, OCBBF2004235, OCBBF2004259, OCBBF2004385, OCBBF2004418,

OCBBF2004478, OCBBF2004482, OCBBF2004533, OCBBF2004567, OCBBF2004612, OCBBF2004647, OCBBF2004669, OCBBF2004826, OCBBF2004866, OCBBF2004883, OCBBF2004889, OCBBF2004930, OCBBF2004984, OCBBF2005066, OCBBF2005077, OCBBF2005161, OCBBF2005343, OCBBF2005349, OCBBF2005373, OCBBF2005420, OCBBF2005428, OCBBF2005433, OCBBF2005546, OCBBF2005843, OCBBF2005901, OCBBF2006005, OCBBF2006030, OCBBF2006058, OCBBF2006113, OCBBF2006148, OCBBF2006151, OCBBF2006154, OCBBF2006172, OCBBF2006214, OCBBF2006241, OCBBF2006388, OCBBF2006438, OCBBF2006567, OCBBF2006624, OCBBF2006639, OCBBF2006660, OCBBF2006764, OCBBF2006859, OCBBF2006972, OCBBF2006987, OCBBF2007039, OCBBF2007051, OCBBF2007068, OCBBF2007114, OCBBF2007121,

OCBBF2007184, OCBBF2007194, OCBBF2007196, OCBBF2007224, OCBBF2007238, OCBBF2007354, OCBBF2007414, OCBBF2007428, OCBBF2007478, OCBBF2007485, OCBBF2007520, OCBBF2007555, OCBBF2007610, OCBBF2007622, OCBBF2007756, OCBBF2007762, OCBBF2007892, OCBBF2007931, OCBBF2007946, OCBBF2008041, OCBBF2008116, OCBBF2008138, OCBBF2008283, OCBBF2008330, OCBBF2008340, OCBBF2008366, OCBBF2008466, OCBBF2008483, OCBBF2008511, OCBBF2008520, OCBBF2008569, OCBBF2008586, OCBBF2008640, OCBBF2008691, OCBBF2008701, OCBBF2008724, OCBBF2008760, OCBBF2008768, OCBBF2008770, OCBBF2008775, OCBBF2008790, OCBBF2008814, OCBBF2008822, OCBBF2009095, OCBBF2009115, OCBBF2009352, OCBBF2009391, OCBBF2009536, OCBBF2009571, OCBBF2009583,

OCBBF2009603, OCBBF2009788, OCBBF2009836, OCBBF2009920, OCBBF2009926, OCBBF2010040, OCBBF2010140, OCBBF2010281, OCBBF2010404, OCBBF2010416, OCBBF2010420, OCBBF2010604, OCBBF2010709, OCBBF2010792, OCBBF2010819, OCBBF2010841, OCBBF2010843, OCBBF2010858, OCBBF2010863, OCBBF2010871, OCBBF2010931, OCBBF2010978, OCBBF2010989, OCBBF2011073, OCBBF2011137, OCBBF2011160, OCBBF2011177, OCBBF2011283, OCBBF2011311, OCBBF2011625, OCBBF2011669, OCBBF2011685, OCBBF2011706, OCBBF2011722, OCBBF2011767, OCBBF2011855, OCBBF2011872, OCBBF2011887, OCBBF2011897, OCBBF2011914, OCBBF2011960, OCBBF2011981, OCBBF2012001, OCBBF2012028, OCBBF2012039, OCBBF2012095, OCBBF2012139, OCBBF2012191, OCBBF2012262, OCBBF2012320,

OCBBF2012436, OCBBF2012525, OCBBF2012553, OCBBF2012678, OCBBF2012704, OCBBF2012714, OCBBF2012755, OCBBF2012812, OCBBF2012821, OCBBF2012881, OCBBF2012908, OCBBF2012936, OCBBF2012990, OCBBF2013011, OCBBF2013049, OCBBF2013091, OCBBF2013123, OCBBF2013127, OCBBF2013208, OCBBF2013285, OCBBF2013319, OCBBF2013324, OCBBF2013366, OCBBF2013481, OCBBF2013507, OCBBF2013721, OCBBF2013789, OCBBF2013883, OCBBF2013926, OCBBF2014052, OCBBF2014089, OCBBF2014322, OCBBF2014386, OCBBF2014541, OCBBF2014576, OCBBF2014707, OCBBF2014745, OCBBF2014828, OCBBF2014873, OCBBF2014880, OCBBF2014928, OCBBF2015031, OCBBF2015081, OCBBF2015115, OCBBF2015232, OCBBF2015233, OCBBF2015285, OCBBF2015334, OCBBF2015335, OCBBF2015503,

OCBBF2015506, OCBBF2015599, OCBBF2015645, OCBBF2015659, OCBBF2015751, OCBBF2015797, OCBBF2015931, OCBBF2015980, OCBBF2016038, OCBBF2016405, OCBBF2016467, OCBBF2016590, OCBBF2016591, OCBBF2016612, OCBBF2016689, OCBBF2016690, OCBBF2016729, OCBBF2016841, OCBBF2016928, OCBBF2017035, OCBBF2017055, OCBBF2017069, OCBBF2017306, OCBBF2017325, OCBBF2017458, OCBBF2017489, OCBBF2017516, OCBBF2017665, OCBBF2017815, OCBBF2017888, OCBBF2017893, OCBBF2017920, OCBBF2018014, OCBBF2018084, OCBBF2018167, OCBBF2018206, OCBBF2018229, OCBBF2018234, OCBBF2018362, OCBBF2018380, OCBBF2018581, OCBBF2018618, OCBBF2018663, OCBBF2018687, OCBBF2018707, OCBBF2018827, OCBBF2018828, OCBBF2018934, OCBBF2019108, OCBBF2019195,

OCBBF2019327, OCBBF2019684, OCBBF2019761, OCBBF2019823, OCBBF2019950, OCBBF2020048, OCBBF2020318, OCBBF2020343, OCBBF2020453, OCBBF2020801, OCBBF2020825, OCBBF2020838, OCBBF2021020, OCBBF2021214, OCBBF2021286, OCBBF2021323, OCBBF2021518, OCBBF2021788, OCBBF2022351, OCBBF2022573, OCBBF2022574, OCBBF2023162, OCBBF2023598, OCBBF2023643, OCBBF2024463, OCBBF2024589, OCBBF2024719, OCBBF2024779, OCBBF2024781, OCBBF2025028, OCBBF2025115, OCBBF2025198, OCBBF2025451, OCBBF2025458, OCBBF2025527, OCBBF2025631, OCBBF2025730, OCBBF2026025, OCBBF2026035, OCBBF2026144, OCBBF2026645, OCBBF2026649, OCBBF2026690, OCBBF2026981, OCBBF2027197, OCBBF2027354, OCBBF2027423, OCBBF2027478, OCBBF2027481, OCBBF2027511,

OCBBF2027661, OCBBF2027728, OCBBF2027782, OCBBF2027868, OCBBF2028055, OCBBF2028173, OCBBF2028421, OCBBF2029471, OCBBF2029901, OCBBF2030116, OCBBF2030329, OCBBF2030354, OCBBF2030517, OCBBF2030574, OCBBF2030708, OCBBF2030927, OCBBF2030963, OCBBF2031167, OCBBF2031198, OCBBF2031366, OCBBF2031561, OCBBF2032152, OCBBF2032274, OCBBF2032539, OCBBF2032590, OCBBF2032599, OCBBF2032611, OCBBF2032671, OCBBF2033295, OCBBF2033413, OCBBF2033869, OCBBF2033948, OCBBF2034333, OCBBF2034529, OCBBF2034637, OCBBF2034906, OCBBF2035110, OCBBF2035214, OCBBF2035226, OCBBF2035390, OCBBF2035564, OCBBF2035658, OCBBF2035823, OCBBF2035885, OCBBF2035916, OCBBF2036019, OCBBF2036031, OCBBF2036225, OCBBF2036476, OCBBF2036752,

OCBBF2037068, OCBBF2037340, OCBBF2037398, OCBBF2037464, OCBBF2037547, OCBBF2037598, OCBBF2037638, OCBBF2038238, OCBBF2038285, OCBBF2038317, OCBBF3000097, OCBBF3000213, OCBBF3000296, OCBBF3000323, OCBBF3000372, OCBBF3000380, OCBBF3000743, OCBBF3000830, OCBBF3001076, OCBBF3001202, OCBBF3001616, OCBBF3002553, OCBBF3002600, OCBBF3002654, OCBBF3003103, OCBBF3003209, OCBBF3003592, OCBBF3003745, OCBBF3003761, OCBBF3004264, OCBBF3004314, OCBBF3004972, OCBBF3005330, OCBBF3005597, OCBBF3005843, OCBBF3006802, OCBBF3006986, OCBBF3007078, OCBBF3007704, OCBBF3008230, OCBBF3008392, OCBBF3008835, OCBBF3009192, OCBBF3009244, OCBBF3009279, PANCR1000012, PANCR1000021, PANCR1000081, PANCR1000086, PANCR1000091,

PANCR1000185, PEBLM1000024, PEBLM1000029, PEBLM1000034, PEBLM1000062, PEBLM1000066, PEBLM1000068, PEBLM1000071, PEBLM1000083, PEBLM1000147, PEBLM1000174, PEBLM1000180, PEBLM2000112, PEBLM2000126, PEBLM2000170, PEBLM2000180, PEBLM2000213, PEBLM2000222, PEBLM2000248, PEBLM2000267, PEBLM2000270, PEBLM2000308, PEBLM2000321, PEBLM2000333, PEBLM2000338, PEBLM2000395, PEBLM2000479, PEBLM2000502, PEBLM2001072, PEBLM2001312, PEBLM2001465, PEBLM2001488, PEBLM2001803, PEBLM2002432, PEBLM2002455, PEBLM2002594, PEBLM2002749, PEBLM2002887, PEBLM2003426, PEBLM2003765, PEBLM2003914, PEBLM2003935, PEBLM2004015, PEBLM2004216, PEBLM2004290, PEBLM2004497, PEBLM2004666, PEBLM2004733, PEBLM2005282, PEBLM2005615,

PEBLM2005632, PEBLM2005697, PEBLM2006031, PEBLM2006036, PEBLM2006049, PEBLM2006113, PEBLM2006135, PEBLM2006215, PEBLM2006283, PEBLM2006298, PEBLM2006912, PEBLM2007112, PEBLM2007140, PEBLM2007188, PEBLM2007296, PEBLM2007437, PEBLM2007447, PEBLM2007598, PEBLM2007774, PEBLM2007832, PEBLM2007834, PEBLM2008106, PEBLM2008576, PEBLM2008605, PEBLM2008826, PEBLM2008861, PERIC1000025, PERIC1000147, PERIC2000180, PERIC2000214, PERIC2000386, PERIC2000387, PERIC2000422, PERIC2000473, PERIC2000478, PERIC2000889, PERIC2000914, PERIC2001227, PERIC2001228, PERIC2001379, PERIC2001504, PERIC2001703, PERIC2001764, PERIC2002243, PERIC2002272, PERIC2002452, PERIC2002766, PERIC2003001, PERIC2003090, PERIC2003287,

PERIC2003349, PERIC2003452, PERIC2003699, PERIC2003720, PERIC2003794, PERIC2003834, PERIC2003919, PERIC2003957, PERIC2004028, PERIC2004131, PERIC2004259, PERIC2004379, PERIC2004429, PERIC2004510, PERIC2004613, PERIC2004909, PERIC2005111, PERIC2005117, PERIC2005305, PERIC2005347, PERIC2005370, PERIC2005936, PERIC2006035, PERIC2006121, PERIC2006945, PERIC2006960, PERIC2007439, PERIC2007914, PERIC2008008, PERIC2008385, PERIC2009001, PERIC2009086, PERIC2009329, PERIC2009566, PLACE5000001, PLACE5000037, PLACE5000058, PLACE5000066, PLACE5000067, PLACE5000068, PLACE5000070, PLACE5000080, PLACE5000105, PLACE5000115, PLACE5000116, PLACE5000129, PLACE5000139, PLACE5000153, PLACE5000171, PLACE5000172,

PLACE5000178, PLACE5000245, PLACE5000260, PLACE5000282, PLACE5000297, PLACE5000492, PLACE5000522, PLACE5000527, PLACE6000012, PLACE6000044, PLACE6000055, PLACE6000070, PLACE6000137, PLACE6000191, PLACE6000205, PLACE6000231, PLACE6000296, PLACE6000314, PLACE6000325, PLACE6000348, PLACE6000371, PLACE6000424, PLACE6000426, PLACE6000429, PLACE6000450, PLACE6000463, PLACE6000523, PLACE6000550, PLACE6000552, PLACE6000555, PLACE6000619, PLACE6000630, PLACE6000953, PLACE6001118, PLACE6001153, PLACE6001262, PLACE6001294, PLACE6001536, PLACE6001557, PLACE6001712, PLACE6001740, PLACE6001823, PLACE6001923, PLACE6001925, PLACE6001933, PLACE6002011, PLACE6002016, PLACE6002056, PLACE6002157, PLACE6002312,

PLACE6002323, PLACE6002345, PLACE6002419, PLACE6002462, PLACE6002647, PLACE6002692, PLACE6002699, PLACE6002710, PLACE6002871, PLACE6002949, PLACE6003004, PLACE6003038, PLACE6003077, PLACE6003094, PLACE6003109, PLACE6003204, PLACE6003372, PLACE6003393, PLACE6003399, PLACE6003539, PLACE6003621, PLACE6003705, PLACE6003740, PLACE6003745, PLACE6003771, PLACE6003796, PLACE6003839, PLACE6003850, PLACE6004101, PLACE6004219, PLACE6004336, PLACE6004368, PLACE6004380, PLACE6004396, PLACE6004454, PLACE6004464, PLACE6004578, PLACE6004663, PLACE6004687, PLACE6004931, PLACE6004966, PLACE6004993, PLACE6005007, PLACE6005029, PLACE6005108, PLACE6005231, PLACE6005234, PLACE6005283, PLACE6005294, PLACE6005328,

PLACE6005351, PLACE6005423, PLACE6005487, PLACE6005535, PLACE6005546, PLACE6005559, PLACE6005579, PLACE6005691, PLACE6005786, PLACE6006077, PLACE6006158, PLACE6006217, PLACE6006222, PLACE6006266, PLACE6006287, PLACE6006394, PLACE6006418, PLACE6006474, PLACE6006566, PLACE6006697, PLACE6006822, PLACE6006871, PLACE6006905, PLACE6006988, PLACE6007050, PLACE6007180, PLACE6007239, PLACE6007242, PLACE6007309, PLACE6007373, PLACE6007380, PLACE6007433, PLACE6007436, PLACE6007482, PLACE6007687, PLACE6007787, PLACE6007798, PLACE6007807, PLACE6007925, PLACE6008036, PLACE6008094, PLACE6008126, PLACE6008285, PLACE6008444, PLACE6008453, PLACE6008640, PLACE6008750, PLACE6008768, PLACE6008793, PLACE6008989,

PLACE6009006, PLACE6009008, PLACE6009228, PLACE6009237, PLACE6009419, PLACE6009430, PLACE6009482, PLACE6009524, PLACE6009527, PLACE6009560, PLACE6009590, PLACE6009821, PLACE6009835, PLACE6009853, PLACE6010463, PLACE6010484, PLACE6010485, PLACE6010568, PLACE6010701, PLACE6010704, PLACE6010732, PLACE6010765, PLACE6010848, PLACE6010925, PLACE6010936, PLACE6010991, PLACE6011041, PLACE6011102, PLACE6011156, PLACE6011211, PLACE6011260, PLACE6011334, PLACE6011627, PLACE6011774, PLACE6011881, PLACE6011907, PLACE6011970, PLACE6011975, PLACE6012028, PLACE6012108, PLACE6012168, PLACE6012297, PLACE6012308, PLACE6012574, PLACE6012908, PLACE6012936, PLACE6013222, PLACE6013232, PLACE6013288, PLACE6013386,

PLACE6013400, PLACE6013650, PLACE6013672, PLACE6013883, PLACE6013884, PLACE6013885, PLACE6014043, PLACE6014064, PLACE6014516, PLACE6014882, PLACE6015051, PLACE6015211, PLACE6015322, PLACE6015445, PLACE6015460, PLACE6015513, PLACE6015591, PLACE6015729, PLACE6015939, PLACE6016030, PLACE6016093, PLACE6016105, PLACE6016160, PLACE6016210, PLACE6016254, PLACE6016942, PLACE6016984, PLACE6017002, PLACE6017564, PLACE6017578, PLACE6017626, PLACE6017701, PLACE6017714, PLACE6017788, PLACE6017791, PLACE6018031, PLACE6018107, PLACE6018187, PLACE6018235, PLACE6018287, PLACE6018441, PLACE6018487, PLACE6018497, PLACE6018769, PLACE6018834, PLACE6018843, PLACE6018863, PLACE6019195, PLACE6019385, PLACE6019542,

PLACE6019600, PLACE6019674, PLACE6019676, PLACE6019701, PLACE6019932, PLACE6020031, PLACE6020145, PLACE7000167, PLACE7000170, PLACE7000266, PLACE7000333, PLACE7000410, PLACE7000502, PLACE7000707, PLACE7001022, PLACE7001544, PLACE7001759, PLACE7001936, PLACE7002303, PLACE7002641, PLACE7003639, PLACE7003657, PLACE7003684, PLACE7003985, PLACE7004103, PLACE7004961, PLACE7005169, PLACE7005671, PLACE7005840, PLACE7006051, PLACE7006090, PLACE7006268, PLACE7006275, PLACE7006498, PLACE7006540, PLACE7007379, PLACE7007644, PLACE7007973, PLACE7008136, PLACE7008261, PLACE7008431, PLACE7008623, PLACE7008766, PLACE7009563, PROST1000033, PROST1000039, PROST1000065, PROST1000083, PROST1000085, PROST1000110,

PROST1000120, PROST1000123, PROST1000136, PROST1000144, PROST1000145, PROST1000152, PROST1000167, PROST1000184, PROST1000215, PROST1000217, PROST1000262, PROST1000272, PROST1000298, PROST1000322, PROST1000334, PROST1000343, PROST1000362, PROST1000377, PROST1000383, PROST1000412, PROST1000419, PROST1000451, PROST1000480, PROST1000485, PROST1000493, PROST1000512, PROST1000527, PROST1000528, PROST1000536, PROST1000559, PROST1000575, PROST2000036, PROST2000053, PROST2000067, PROST2000079, PROST2000105, PROST2000143, PROST2000162, PROST2000165, PROST2000176, PROST2000203, PROST2000206, PROST2000212, PROST2000241, PROST2000267, PROST2000274, PROST2000277, PROST2000284, PROST2000325, PROST2000337,

PROST2000432, PROST2000452, PROST2000463, PROST2000470, PROST2000505, PROST2000567, PROST2000572, PROST2000717, PROST2000760, PROST2000761, PROST2000835, PROST2000893, PROST2000961, PROST2001002, PROST2001116, PROST2001138, PROST2001180, PROST2001213, PROST2001283, PROST2001414, PROST2001415, PROST2001465, PROST2001521, PROST2001540, PROST2001552, PROST2001599, PROST2001611, PROST2001636, PROST2001739, PROST2001796, PROST2001805, PROST2001823, PROST2001883, PROST2001899, PROST2001997, PROST2001998, PROST2002078, PROST2002101, PROST2002162, PROST2002212, PROST2002227, PROST2002338, PROST2002425, PROST2002488, PROST2002489, PROST2002527, PROST2002591, PROST2002602, PROST2002633, PROST2002682,

PROST2002685, PROST2002722, PROST2002736, PROST2002842, PROST2002897, PROST2002906, PROST2002927, PROST2002960, PROST2003102, PROST2003117, PROST2003210, PROST2003302, PROST2003303, PROST2003324, PROST2003340, PROST2003428, PROST2003472, PROST2003517, PROST2003537, PROST2003563, PROST2003577, PROST2003583, PROST2003586, PROST2003628, PROST2003635, PROST2003674, PROST2003676, PROST2003725, PROST2003732, PROST2003775, PROST2003922, PROST2003930, PROST2004115, PROST2004142, PROST2004146, PROST2004161, PROST2004251, PROST2004332, PROST2004481, PROST2004526, PROST2004570, PROST2004727, PROST2004739, PROST2004742, PROST2004744, PROST2004817, PROST2005039, PROST2005067, PROST2005076, PROST2005093,

PROST2005121, PROST2005131, PROST2005143, PROST2005228, PROST2005272, PROST2005426, PROST2005466, PROST2005630, PROST2005683, PROST2005788, PROST2005793, PROST2005880, PROST2005886, PROST2005904, PROST2005919, PROST2005943, PROST2006008, PROST2006020, PROST2006060, PROST2006196, PROST2006201, PROST2006260, PROST2006282, PROST2006343, PROST2006450, PROST2006491, PROST2006510, PROST2006579, PROST2006688, PROST2006766, PROST2006782, PROST2006933, PROST2006988, PROST2007122, PROST2007237, PROST2007248, PROST2007289, PROST2007317, PROST2007328, PROST2007382, PROST2007389, PROST2007416, PROST2007528, PROST2007612, PROST2007818, PROST2007871, PROST2007950, PROST2007956, PROST2007974, PROST2007988,

PROST2008079, PROST2008088, PROST2008243, PROST2008245, PROST2008268, PROST2008360, PROST2008447, PROST2008468, PROST2008472, PROST2008516, PROST2008724, PROST2008835, PROST2008993, PROST2009022, PROST2009208, PROST2009222, PROST2009254, PROST2009273, PROST2009320, PROST2009347, PROST2009365, PROST2009388, PROST2009400, PROST2009470, PROST2009483, PROST2009571, PROST2009731, PROST2009736, PROST2009795, PROST2009901, PROST2009909, PROST2010046, PROST2010219, PROST2010250, PROST2010326, PROST2010382, PROST2010400, PROST2010579, PROST2010606, PROST2010885, PROST2011012, PROST2011038, PROST2011105, PROST2011297, PROST2011410, PROST2011439, PROST2011482, PROST2011577, PROST2011631, PROST2011660,

PROST2012005, PROST2012016, PROST2012088, PROST2012140, PROST2012157, PROST2012190, PROST2012249, PROST2012353, PROST2012400, PROST2012504, PROST2012542, PROST2012550, PROST2012740, PROST2012745, PROST2012780, PROST2012888, PROST2013032, PROST2013053, PROST2013121, PROST2013260, PROST2013327, PROST2013531, PROST2013605, PROST2013627, PROST2013873, PROST2013880, PROST2014422, PROST2014585, PROST2014601, PROST2014659, PROST2014862, PROST2014925, PROST2015124, PROST2015137, PROST2015198, PROST2015243, PROST2015246, PROST2015251, PROST2015287, PROST2015332, PROST2015457, PROST2015464, PROST2015537, PROST2015545, PROST2015551, PROST2015636, PROST2015710, PROST2015756, PROST2015877, PROST2015900,

PROST2015932, PROST2016195, PROST2016246, PROST2016351, PROST2016379, PROST2016444, PROST2016462, PROST2016499, PROST2016512, PROST2016566, PROST2016582, PROST2016668, PROST2016684, PROST2016777, PROST2016829, PROST2016860, PROST2016980, PROST2017098, PROST2017128, PROST2017367, PROST2017413, PROST2017441, PROST2017529, PROST2017578, PROST2017612, PROST2017700, PROST2017701, PROST2017729, PROST2017836, PROST2017910, PROST2017972, PROST2018030, PROST2018090, PROST2018235, PROST2018437, PROST2018487, PROST2018511, PROST2018583, PROST2018588, PROST2018607, PROST2018788, PROST2018902, PROST2018922, PROST2019164, PROST2019253, PROST2019296, PROST2019398, PROST2019487, PROST2019781, PUAEN1000039,

PUAEN1000081, PUAEN1000085, PUAEN1000087, PUAEN1000121, PUAEN1000147, PUAEN1000161, PUAEN1000175, PUAEN1000275, PUAEN1000276, PUAEN1000288, PUAEN1000322, PUAEN2000080, PUAEN2000152, PUAEN2000175, PUAEN2000247, PUAEN2000312, PUAEN2000394, PUAEN2000497, PUAEN2000535, PUAEN2000594, PUAEN2000684, PUAEN2000942, PUAEN2001260, PUAEN2001345, PUAEN2001526, PUAEN2001882, PUAEN2002473, PUAEN2002489, PUAEN2002552, PUAEN2002568, PUAEN2002616, PUAEN2002758, PUAEN2003018, PUAEN2003079, PUAEN2003116, PUAEN2003408, PUAEN2003517, PUAEN2003954, PUAEN2004067, PUAEN2004083, PUAEN2004400, PUAEN2004511, PUAEN2004525, PUAEN2004875, PUAEN2005110, PUAEN2005247, PUAEN2005502, PUAEN2005588, PUAEN2005930, PUAEN2006029,

PUAEN2006328, PUAEN2006335, PUAEN2006578, PUAEN2006639, PUAEN2006761, PUAEN2007350, PUAEN2007785, PUAEN2007898, PUAEN2008123, PUAEN2008314, PUAEN2008515, PUAEN2008815, PUAEN2008939, PUAEN2008980, PUAEN2008985, PUAEN2009174, PUAEN2009348, PUAEN2009534, PUAEN2009795, PUAEN2009852, PUAEN2010346, PUAEN2010824, RECTM1000051, RECTM1000141, RECTM2000220, RECTM2000349, RECTM2000433, RECTM2000510, RECTM2001307, RECTM2001347, RECTM2001519, RECTM2001666, RECTM2001691, RECTM2001749, RECTM2002172, RECTM2002602, RECTM2002632, SALGL1000005, SALGL1000024, SALGL1000028, SALGL1000047, SALGL1000065, SALGL1000107, SALGL1000171, SKMUS1000030, SKMUS1000064, SKMUS1000067, SKMUS1000102, SKMUS1000104, SKMUS1000110,

SKMUS1000118, SKMUS1000124, SKMUS1000129, SKMUS1000176, SKMUS2000061, SKMUS2000074, SKMUS2000078, SKMUS2000117, SKMUS2000137, SKMUS2000198, SKMUS2000243, SKMUS2000271, SKMUS2000287, SKMUS2000317, SKMUS2000330, SKMUS2000339, SKMUS2000365, SKMUS2000390, SKMUS2000416, SKMUS2000458, SKMUS2000467, SKMUS2000468, SKMUS2000484, SKMUS2000515, SKMUS2000679, SKMUS2000690, SKMUS2000701, SKMUS2000705, SKMUS2000710, SKMUS2000724, SKMUS2000726, SKMUS2000757, SKMUS2000774, SKMUS2000780, SKMUS2000821, SKMUS2000847, SKMUS2000863, SKMUS2000894, SKMUS2000898, SKMUS2000933, SKMUS2000945, SKMUS2000961, SKMUS2000973, SKMUS2000980, SKMUS2001008, SKMUS2001014, SKMUS2001147, SKMUS2001157, SKMUS2001164, SKMUS2001199,

SKMUS2001201, SKMUS2001215, SKMUS2001247, SKMUS2001254, SKMUS2001271, SKMUS2001287, SKMUS2001323, SKMUS2001364, SKMUS2001398, SKMUS2001402, SKMUS2001412, SKMUS2001492, SKMUS2001528, SKMUS2001543, SKMUS2001580, SKMUS2001585, SKMUS2001608, SKMUS2001631, SKMUS2001634, SKMUS2001662, SKMUS2001663, SKMUS2001668, SKMUS2001671, SKMUS2001683, SKMUS2001693, SKMUS2001695, SKMUS2001703, SKMUS2001740, SKMUS2001742, SKMUS2001823, SKMUS2001850, SKMUS2002077, SKMUS2002084, SKMUS2002153, SKMUS2002475, SKMUS2002602, SKMUS2002634, SKMUS2002693, SKMUS2002821, SKMUS2002840, SKMUS2002920, SKMUS2003034, SKMUS2003074, SKMUS2003168, SKMUS2003194, SKMUS2003744, SKMUS2004044, SKMUS2004047, SKMUS2004483, SKMUS2004667,

SKMUS2004897, SKMUS2004903, SKMUS2004908, SKMUS2005428, SKMUS2006394, SKMUS2006481, SKMUS2006755, SKMUS2007315, SKMUS2007359, SKMUS2007568, SKMUS2007740, SKMUS2007816, SKMUS2007915, SKMUS2008338, SKMUS2008407, SKMUS2008474, SKMUS2008585, SKMUS2008607, SKMUS2009190, SKMUS2009232, SKMUS2009479, SKMUS2009557, SKNMC1000123, SKNMC1000124, SKNMC1000159, SKNMC1000229, SKNMC1000264, SKNMC2000224, SKNMC2000256, SKNMC2000322, SKNMC2000356, SKNMC2000374, SKNMC2000583, SKNMC2000593, SKNMC2000612, SKNMC2000622, SKNMC2000698, SKNMC2000966, SKNMC2001113, SKNMC2001324, SKNMC2001503, SKNMC2001555, SKNMC2001596, SKNMC2001623, SKNMC2002402, SKNMC2003639, SKNMC2003924, SKNMC2003987, SKNMC2004457, SKNMC2004643,

SKNMC2004651, SKNMC2005772, SKNMC2006173, SKNMC2006327, SKNMC2006998, SKNMC2007134, SKNMC2007502, SKNMC2007504, SKNMC2007961, SKNMC2009450, SKNSH1000002, SKNSH1000101, SKNSH1000174, SKNSH1000301, SKNSH1000308, SKNSH1000416, SKNSH2000051, SKNSH2000101, SKNSH2000151, SKNSH2000163, SKNSH2000250, SKNSH2000319, SKNSH2000516, SKNSH2000550, SKNSH2000716, SKNSH2000971, SKNSH2001222, SKNSH2001875, SKNSH2001931, SKNSH2002054, SKNSH2002768, SKNSH2002866, SKNSH2003064, SKNSH2003174, SKNSH2003422, SKNSH2003466, SKNSH2003483, SKNSH2003633, SKNSH2004039, SKNSH2005061, SKNSH2005120, SKNSH2005194, SKNSH2005240, SKNSH2005792, SKNSH2005816, SKNSH2005981, SKNSH2006234, SKNSH2006304, SKNSH2006432, SKNSH2006822,

SKNSH2007142, SKNSH2007149, SKNSH2007429, SKNSH2007739, SKNSH2008940, SKNSH2008969, SKNSH2009197, SKNSH2009357, SKNSH2009435, SKNSH2009991, SKNSH2010015, SMINT1000012, SMINT1000016, SMINT1000039, SMINT1000048, SMINT1000051, SMINT1000054, SMINT1000057, SMINT1000071, SMINT1000075, SMINT1000078, SMINT1000091, SMINT1000103, SMINT1000118, SMINT1000137, SMINT1000192, SMINT2000007, SMINT2000018, SMINT2000032, SMINT2000040, SMINT2000060, SMINT2000073, SMINT2000084, SMINT2000145, SMINT2000176, SMINT2000185, SMINT2000227, SMINT2000229, SMINT2000232, SMINT2000239, SMINT2000267, SMINT2000400, SMINT2000454, SMINT2000518, SMINT2000529, SMINT2000530, SMINT2000537, SMINT2000538, SMINT2000541, SMINT2000558,

SMINT2000562, SMINT2000568, SMINT2000602, SMINT2000609, SMINT2000659, SMINT2000694, SMINT2000747, SMINT2000811, SMINT2000824, SMINT2000933, SMINT2000984, SMINT2001114, SMINT2001158, SMINT2001183, SMINT2001199, SMINT2001222, SMINT2001348, SMINT2001397, SMINT2001458, SMINT2001461, SMINT2001535, SMINT2001559, SMINT2001609, SMINT2001615, SMINT2001683, SMINT2001731, SMINT2001812, SMINT2001818, SMINT2002126, SMINT2002202, SMINT2002210, SMINT2002224, SMINT2002281, SMINT2002311, SMINT2002328, SMINT2002414, SMINT2002457, SMINT2002620, SMINT2002743, SMINT2002880, SMINT2002882, SMINT2002884, SMINT2002899, SMINT2002974, SMINT2003003, SMINT2003074, SMINT2003128, SMINT2003150, SMINT2003317, SMINT2003340,

SMINT2003386, SMINT2003423, SMINT2003505, SMINT2003551, SMINT2003569, SMINT2003641, SMINT2003644, SMINT2003866, SMINT2003905, SMINT2004023, SMINT2004037, SMINT2004047, SMINT2004086, SMINT2004144, SMINT2004280, SMINT2004299, SMINT2004339, SMINT2004350, SMINT2004414, SMINT2004473, SMINT2004547, SMINT2004583, SMINT2004729, SMINT2004781, SMINT2004872, SMINT2004891, SMINT2004909, SMINT2004972, SMINT2004992, SMINT2005075, SMINT2005161, SMINT2005174, SMINT2005213, SMINT2005330, SMINT2005387, SMINT2005402, SMINT2005405, SMINT2005621, SMINT2005623, SMINT2005624, SMINT2005688, SMINT2005800, SMINT2006078, SMINT2006205, SMINT2006331, SMINT2006596, SMINT2006641, SMINT2006648, SMINT2006708, SMINT2006716,

SMINT2007062, SMINT2007101, SMINT2007140, SMINT2007219, SMINT2007294, SMINT2007365, SMINT2007391, SMINT2007433, SMINT2007508, SMINT2007526, SMINT2007579, SMINT2007647, SMINT2007729, SMINT2007790, SMINT2007792, SMINT2007796, SMINT2007867, SMINT2007932, SMINT2008042, SMINT2008054, SMINT2008133, SMINT2008277, SMINT2008329, SMINT2008455, SMINT2008491, SMINT2008531, SMINT2008545, SMINT2008558, SMINT2008590, SMINT2008625, SMINT2008672, SMINT2008690, SMINT2008715, SMINT2008720, SMINT2008817, SMINT2008844, SMINT2008869, SMINT2008917, SMINT2008921, SMINT2009119, SMINT2009121, SMINT2009212, SMINT2009216, SMINT2009233, SMINT2009259, SMINT2009272, SMINT2009292, SMINT2009363, SMINT2009415, SMINT2009468,

SMINT2009505, SMINT2009529, SMINT2009832, SMINT2009895, SMINT2009902, SMINT2009973, SMINT2010015, SMINT2010068, SMINT2010076, SMINT2010084, SMINT2010144, SMINT2010200, SMINT2010278, SMINT2010324, SMINT2010326, SMINT2010369, SMINT2010426, SMINT2010500, SMINT2010533, SMINT2010629, SMINT2010639, SMINT2010672, SMINT2010753, SMINT2010853, SMINT2010897, SMINT2010901, SMINT2010997, SMINT2011066, SMINT2011273, SMINT2011311, SMINT2011406, SMINT2011509, SMINT2011567, SMINT2011588, SMINT2011656, SMINT2011888, SMINT2011958, SMINT2012040, SMINT2012179, SMINT2012195, SMINT2012285, SMINT2012291, SMINT2012501, SMINT2012734, SMINT2012793, SMINT2013032, SMINT2013129, SMINT2013170, SMINT2013181, SMINT2013228,

SMINT2013613, SMINT2013626, SMINT2013833, SMINT2013890, SMINT2014166, SMINT2014282, SMINT2014443, SMINT2014489, SMINT2014721, SMINT2015006, SMINT2015294, SMINT2015306, SMINT2015326, SMINT2015353, SMINT2015454, SMINT2015518, SMINT2015745, SMINT2015787, SMINT2015810, SMINT2016122, SMINT2016146, SMINT2016286, SMINT2016313, SMINT2016396, SMINT2016412, SMINT2016440, SMINT2016477, SMINT2016544, SMINT2016857, SMINT2017134, SMINT2017319, SMINT2017324, SMINT2017410, SMINT2017431, SMINT2017432, SMINT2017436, SMINT2017599, SMINT2017659, SMINT2017736, SMINT2017781, SMINT2017855, SMINT2017884, SMINT2017964, SMINT2017974, SMINT2018343, SMINT2018353, SMINT2018592, SMINT2019017, SMINT2019105, SMINT2019142,

SMINT2019153, SMINT2019200, SPLEN1000013, SPLEN1000041, SPLEN1000049, SPLEN1000056, SPLEN1000087, SPLEN1000089, SPLEN1000091, SPLEN1000134, SPLEN1000139, SPLEN1000143, SPLEN1000144, SPLEN1000145, SPLEN1000156, SPLEN1000163, SPLEN1000165, SPLEN1000178, SPLEN2000020, SPLEN2000047, SPLEN2000064, SPLEN2000072, SPLEN2000098, SPLEN2000126, SPLEN2000134, SPLEN2000143, SPLEN2000189, SPLEN2000197, SPLEN2000205, SPLEN2000210, SPLEN2000222, SPLEN2000242, SPLEN2000247, SPLEN2000258, SPLEN2000267, SPLEN2000270, SPLEN2000307, SPLEN2000310, SPLEN2000341, SPLEN2000348, SPLEN2000350, SPLEN2000357, SPLEN2000364, SPLEN2000402, SPLEN2000414, SPLEN2000420, SPLEN2000443, SPLEN2000448, SPLEN2000450, SPLEN2000464,

SPLEN2000496, SPLEN2000515, SPLEN2000516, SPLEN2000537, SPLEN2000541, SPLEN2000607, SPLEN2000630, SPLEN2000695, SPLEN2000698, SPLEN2000874, SPLEN2000882, SPLEN2001135, SPLEN2001176, SPLEN2001227, SPLEN2001245, SPLEN2001278, SPLEN2001354, SPLEN2001367, SPLEN2001383, SPLEN2001476, SPLEN2001503, SPLEN2001510, SPLEN2001599, SPLEN2001626, SPLEN2001650, SPLEN2001710, SPLEN2001761, SPLEN2001781, SPLEN2001881, SPLEN2001912, SPLEN2001945, SPLEN2002007, SPLEN2002053, SPLEN2002133, SPLEN2002147, SPLEN2002166, SPLEN2002175, SPLEN2002223, SPLEN2002272, SPLEN2002314, SPLEN2002343, SPLEN2002354, SPLEN2002385, SPLEN2002419, SPLEN2002451, SPLEN2002462, SPLEN2002467, SPLEN2002477, SPLEN2002493, SPLEN2002662,

SPLEN2002695, SPLEN2002707, SPLEN2002744, SPLEN2002917, SPLEN2002931, SPLEN2002957, SPLEN2003160, SPLEN2003204, SPLEN2003219, SPLEN2003396, SPLEN2003678, SPLEN2003878, SPLEN2003918, SPLEN2003924, SPLEN2004060, SPLEN2004078, SPLEN2004124, SPLEN2004148, SPLEN2004181, SPLEN2004343, SPLEN2004346, SPLEN2004368, SPLEN2004555, SPLEN2004611, SPLEN2004662, SPLEN2004773, SPLEN2004844, SPLEN2004880, SPLEN2004984, SPLEN2005227, SPLEN2005272, SPLEN2005342, SPLEN2005416, SPLEN2005450, SPLEN2005474, SPLEN2005560, SPLEN2005727, SPLEN2005783, SPLEN2005790, SPLEN2005806, SPLEN2005818, SPLEN2005838, SPLEN2005869, SPLEN2005939, SPLEN2006268, SPLEN2006283, SPLEN2006325, SPLEN2006389, SPLEN2006425, SPLEN2006671,

SPLEN2006701, SPLEN2006785, SPLEN2006875, SPLEN2007182, SPLEN2007276, SPLEN2007314, SPLEN2007326, SPLEN2007350, SPLEN2007619, SPLEN2007653, SPLEN2007689, SPLEN2007695, SPLEN2007739, SPLEN2007750, SPLEN2007794, SPLEN2007879, SPLEN2007926, SPLEN2007951, SPLEN2008007, SPLEN2008135, SPLEN2008164, SPLEN2008317, SPLEN2008460, SPLEN2008496, SPLEN2008591, SPLEN2068786, SPLEN2009081, SPLEN2009088, SPLEN2009163, SPLEN2009340, SPLEN2009512, SPLEN2009513, SPLEN2009541, SPLEN2009548, SPLEN2009555, SPLEN2009581, SPLEN2009733, SPLEN2009803, SPLEN2009884, SPLEN2009918, SPLEN2009970, SPLEN2010004, SPLEN2010015, SPLEN2010112, SPLEN2010119, SPLEN2010146, SPLEN2010187, SPLEN2010350, SPLEN2010395, SPLEN2010415,

SPLEN2010447, SPLEN2010469, SPLEN201051-0, SPLEN2010530, SPLEN2010534, SPLEN2010588, SPLEN2010625, SPLEN2010800, SPLEN2010846, SPLEN2010847, SPLEN2010862, SPLEN2010912, SPLEN2010978, SPLEN2010986, SPLEN2011018, SPLEN2011021, SPLEN2011025, SPLEN2011086, SPLEN2011139, SPLEN2011145, SPLEN2011248, SPLEN2011252, SPLEN2011422, SPLEN2011672, SPLEN2011678, SPLEN2011758, SPLEN2011766, SPLEN2011805, SPLEN2011820, SPLEN2011830, SPLEN2011931, SPLEN2011981, SPLEN2012115, SPLEN2012175, SPLEN2012179, SPLEN2012185, SPLEN2012234, SPLEN2012453, SPLEN2012523, SPLEN2012524, SPLEN2012525, SPLEN2012606, SPLEN2012611, SPLEN2012619, SPLEN2012624, SPLEN2012725, SPLEN2012743, SPLEN2012756, SPLEN2012846, SPLEN2012889,

SPLEN2012961, SPLEN2013108, SPLEN2013195, SPLEN2013503, SPLEN2013644, SPLEN2013648, SPLEN2013670, SPLEN2013673, SPLEN2013714, SPLEN2013753, SPLEN2013821, SPLEN2013857, SPLEN2013860, SPLEN2013910, SPLEN2013923, SPLEN2013936, SPLEN2014041, SPLEN2014063, SPLEN2014073, SPLEN2014119, SPLEN2014136, SPLEN2014199, SPLEN2014210, SPLEN2014293, SPLEN2014318, SPLEN2014381, SPLEN2014452, SPLEN2014454, SPLEN2014486, SPLEN2014543, SPLEN2014645, SPLEN2014669, SPLEN2014711, SPLEN2014739, SPLEN2014860, SPLEN2014865, SPLEN2014911, SPLEN2014919, SPLEN2014920, SPLEN2014924, SPLEN2014937, SPLEN2014946, SPLEN2015031, SPLEN2015094, SPLEN2015121, SPLEN2015129, SPLEN2015158, SPLEN2015166, SPLEN2015216, SPLEN2015258,

SPLEN2015261, SPLEN2015267, SPLEN2015276, SPLEN2015310, SPLEN2015415, SPLEN2015433, SPLEN2015593, SPLEN2015674, SPLEN2015679, SPLEN2015707, SPLEN2015730, SPLEN2015746, SPLEN2015788, SPLEN2015815, SPLEN2015890, SPLEN2015891, SPLEN2015899, SPLEN2016045, SPLEN2016053, SPLEN2016069, SPLEN2016098, SPLEN2016135, SPLEN2016139, SPLEN2016241, SPLEN2016250, SPLEN2016304, SPLEN2016356, SPLEN2016480, SPLEN2016531, SPLEN2016533, SPLEN2016541, SPLEN2016552, SPLEN2016602, SPLEN2016627, SPLEN2016678, SPLEN2016743, SPLEN2016773, SPLEN2016781, SPLEN2016840, SPLEN2016863, SPLEN2016922, SPLEN2016932, SPLEN2016973, SPLEN2017043, SPLEN2017104, SPLEN2017121, SPLEN2017147, SPLEN2017187, SPLEN2017189, SPLEN2017212,

SPLEN2017351, SPLEN2017426, SPLEN2017452, SPLEN2017538, SPLEN2017592, SPLEN2017613, SPLEN2017620, SPLEN2017643, SPLEN2017687, SPLEN2017740, SPLEN2017781, SPLEN2017999, SPLEN2018112, SPLEN2018159, SPLEN2018180, SPLEN2018181, SPLEN2018280, SPLEN2018285, SPLEN2018299, SPLEN2018302, SPLEN2018364, SPLEN2018395, SPLEN2018643, SPLEN2018683, SPLEN2018732, SPLEN2018749, SPLEN2018933, SPLEN2019008, SPLEN2019043, SPLEN2019077, SPLEN2019092, SPLEN2019102, SPLEN2019131, SPLEN2019257, SPLEN2019323, SPLEN2019349, SPLEN2019375, SPLEN2019379, SPLEN2019446, SPLEN2019471, SPLEN2019480, SPLEN2019505, SPLEN2019571, SPLEN2019575, SPLEN2019709, SPLEN2019774, SPLEN2019793, SPLEN2019811, SPLEN2019839, SPLEN2019956,

SPLEN2019985, SPLEN2020007, SPLEN2020034, SPLEN2020153, SPLEN2020154, SPLEN2020166, SPLEN2020183, SPLEN2020223, SPLEN2020359, SPLEN2020417, SPLEN2020427, SPLEN2020467, SPLEN2020512, SPLEN2020608, SPLEN2020777, SPLEN2020805, SPLEN2021057, SPLEN2021122, SPLEN2021157, SPLEN2021231, SPLEN2021295, SPLEN2021298, SPLEN2021383, SPLEN2021417, SPLEN2021440, SPLEN2021458, SPLEN2021463, SPLEN2021560, SPLEN2021701, SPLEN2021795, SPLEN2021983, SPLEN2021991, SPLEN2022040, SPLEN2022162, SPLEN2022227, SPLEN2022591, SPLEN2022785, SPLEN2022920, SPLEN2023024, SPLEN2023118, SPLEN2023160, SPLEN2023423, SPLEN2023710, SPLEN2023733, SPLEN2023791, SPLEN2023977, SPLEN2024070, SPLEN2024127, SPLEN2024232, SPLEN2024273,

SPLEN2024383, SPLEN2024489, SPLEN2024530, SPLEN2024571, SPLEN2024577, SPLEN2024617, SPLEN2024737, SPLEN2024922, SPLEN2024956, SPLEN2024990, SPLEN2025012, SPLEN2025017, SPLEN2025039, SPLEN2025219, SPLEN2025491, SPLEN2025626, SPLEN2025820, SPLEN2026144, SPLEN2026411, SPLEN2026765, SPLEN2027157, SPLEN2027395, SPLEN2027488, SPLEN2027780, SPLEN2027852, SPLEN2027868, SPLEN2027995, SPLEN2028046, SPLEN2028066, SPLEN2028138, SPLEN2028365, SPLEN2028417, SPLEN2028424, SPLEN2028593, SPLEN2028844, SPLEN2028914, SPLEN2028994, SPLEN2029051, SPLEN2029176, SPLEN2029295, SPLEN2029380, SPLEN2029522, SPLEN2029683, SPLEN2029727, SPLEN2029904, SPLEN2029912, SPLEN2030250, SPLEN2030335, SPLEN2030371, SPLEN2030397,

SPLEN2030479, SPLEN2030495, SPLEN2030562, SPLEN2030763, SPLEN2030847, SPLEN2031004, SPLEN2031125, SPLEN2031424, SPLEN2031674, SPLEN2031724, SPLEN2031780, SPLEN2032036, SPLEN2032112, SPLEN2032154, SPLEN2032157, SPLEN2032321, SPLEN2032356, SPLEN2032677, SPLEN2032813, SPLEN2032924, SPLEN2033098, SPLEN2033153, SPLEN2033333, SPLEN2033490, SPLEN2033539, SPLEN2033921, SPLEN2033996, SPLEN2034021, SPLEN2034081, SPLEN2034252, SPLEN2034448, SPLEN2034551, SPLEN2034601, SPLEN2034678, SPLEN2034781, SPLEN2034864, SPLEN2034934, SPLEN2035318, SPLEN2035615, SPLEN2036076, SPLEN2036103, SPLEN2036326, SPLEN2036475, SPLEN2036501, SPLEN2036579, SPLEN2036608, SPLEN2036702, SPLEN2036712, SPLEN2036821, SPLEN2036953,

SPLEN2037077, SPLEN2037194, SPLEN2037319, SPLEN2037444, SPLEN2037580, SPLEN2037630, SPLEN2037678, SPLEN2037722, SPLEN2037891, SPLEN2038055, SPLEN2038180, SPLEN2038345, SPLEN2038407, SPLEN2038473, SPLEN2038834, SPLEN2039311, SPLEN2039672, SPLEN2039697, SPLEN2039845, SPLEN2039936, SPLEN2040222, SPLEN2040237, SPLEN2040824, SPLEN2041304, SPLEN2041306, SPLEN2041310, SPLEN2041645, SPLEN2041700, SPLEN2041720, SPLEN2041977, SPLEN2042051, SPLEN2042303, SPLEN2042521, SPLEN2042535, SPLEN2042598, SPLEN2042920, STOMA1000013, STOMA1000047, STOMA1000052, STOMA1000074, STOMA1000091, STOMA1000117, STOMA1000133, STOMA1000175, STOMA1000190, STOMA1000191, STOMA2000032, STOMA2000072, STOMA2000088, STOMA2000104,

STOMA2000121, STOMA2000148, STOMA2000168, STOMA2000183, STOMA2000188, STOMA2000257, STOMA2000279, STOMA2000383, STOMA2000386, STOMA2000478, STOMA2000482, STOMA2000529, STOMA2000539, STOMA2000567, STOMA2000640, STOMA2000678, STOMA2000686, STOMA2000888, STOMA2001025, STOMA2001389, STOMA2002141, STOMA2002166, STOMA2002367, STOMA2002688, STOMA2002828, STOMA2003158, STOMA2003289, STOMA2003444, STOMA2003477, STOMA2003646, STOMA2003716, STOMA2003781, STOMA2003894, STOMA2004194, STOMA2004294, STOMA2004663, STOMA2004668, STOMA2004852, STOMA2004873, STOMA2004884, STOMA2004893, STOMA2005120, STOMA2005664, STOMA2005667, STOMA2005746, STOMA2005782, STOMA2006181, STOMA2006213, STOMA2006229, STOMA2006325,

STOMA2006330, STOMA2006398, STOMA2006447, STOMA2006780, STOMA2006904, STOMA2007269, STOMA2007680, STOMA2007745, STOMA2008050, STOMA2008361, STOMA2008546, STOMA2008614, STOMA2008638, STOMA2008838, STOMA2009250, STOMA2009253, STOMA2009256, STOMA2009289, SYNOV1000045, SYNOV1000049, SYNOV1000118, SYNOV1000128, SYNOV1000164, SYNOV1000190, SYNOV1000256, SYNOV1000374, SYNOV2000152, SYNOV2000171, SYNOV2000173, SYNOV2000177, SYNOV2000178, SYNOV2000193, SYNOV2000251, SYNOV2000279, SYNOV2000291, SYNOV2000297, SYNOV2000397, SYNOV2000426, SYNOV2000601, SYNOV2000700, SYNOV2000820, SYNOV2000824, SYNOV2000923, SYNOV2001014, SYNOV2001035, SYNOV2001088, SYNOV2001111, SYNOV2001144, SYNOV2001212, SYNOV2001251,

SYNOV2001300, SYNOV2001356, SYNOV2001374, SYNOV2001390, SYNOV2001451, SYNOV2001457, SYNOV2001468, SYNOV2001581, SYNOV2001648, SYNOV2001660, SYNOV2003326, SYNOV2005216, SYNOV2005448, SYNOV2005541, SYNOV2005673, SYNOV2005817, SYNOV2005962, SYNOV2006430, SYNOV2006620, SYNOV2007758, SYNOV2007965, SYNOV2008765, SYNOV2009172, SYNOV2011233, SYNOV2012326, SYNOV2013365, SYNOV2013637, SYNOV2014157, SYNOV2014364, SYNOV2016124, SYNOV2016837, SYNOV2017179, SYNOV2018921, SYNOV2019280, SYNOV2020085, SYNOV2020463, SYNOV2021320, SYNOV2021953, SYNOV3000184, SYNOV3000231, SYNOV3000302, SYNOV3000345, SYNOV4000249, SYNOV4000472, SYNOV4000706, SYNOV4001153, SYNOV4001326, SYNOV4001342, SYNOV4002001, SYNOV4002392,

SYNOV4002875, SYNOV4002883, SYNOV4003174, SYNOV4003322, SYNOV4003681, SYNOV4003981, SYNOV4004184, SYNOV4004741, SYNOV4004823, SYNOV4004914, SYNOV4005256, SYNOV4005570, SYNOV4005739, SYNOV4005889, SYNOV4005981, SYNOV4005989, SYNOV4006256, SYNOV4006327, SYNOV4006542, SYNOV4007012, SYNOV4007215, SYNOV4007360, SYNOV4007430, SYNOV4007521, SYNOV4007553, SYNOV4007671, SYNOV4007711, SYNOV4008245, SYNOV4008336, SYNOV4009129, SYNOV4009139, SYNOV4009298, T1ESE2000116, T1ESE2000609, T1ESE2002665, TBAES2000004, TBAES2000116, TBAES2000255, TBAES2000315, TBAES2000352, TBAES2000932, TBAES2001171, TBAES2001220, TBAES2001229, TBAES2001258, TBAES2001296, TBAES2001492, TBAES2001553, TBAES2001751, TBAES2003435,

TBAES2003492, TBAES2003550, TBAES2003917, TBAES2004055, TBAES2004105, TBAES2004939, TBAES2005157, TBAES2005361, TBAES2005543, TBAES2006568, TBAES2006881, TBAES2007379, TBAES2007428, TBAES2007481, TBAES2007504, TBAES2007548, TBAES2007697, TBAES2007862, TBAES2007964, TBAES2008133, TCERX2000171, TCERX2000431, TCERX2000998, TCERX2002644, TCOLN1000037, TCOLN1000180, TCOLN2000139, TCOLN2000236, TCOLN2000641, TCOLN2001268, TCOLN2002047, TCOLN2002278, TESOP1000035, TESOP1000127, TESOP1000160, TESOP2000083, TESOP2000090, TESOP2000205, TESOP2000312, TESOP2000390, TESOP2000400, TESOP2000510, TESOP2000527, TESOP2000569, TESOP2000739, TESOP2000801, TESOP2001122, TESOP2001166, TESOP2001345, TESOP2001474,

TESOP2001796, TESOP2001818, TESOP2001849, TESOP2001865, TESOP2001901, TESOP2001953, TESOP2002005, TESOP2002110, TESOP2002150, TESOP2002451, TESOP2002489, TESOP2002539, TESOP2002545, TESOP2002804, TESOP2002950, TESOP2003273, TESOP2003308, TESOP2003553, TESOP2003753, TESOP2004110, TESOP2004114, TESOP2004173, TESOP2004214, TESOP2005199, TESOP2005285, TESOP2005485, TESOP2005498, TESOP2005579, TESOP2006041, TESOP2006053, TESOP2006060, TESOP2006068, TESOP2006110, TESOP2006398, TESOP2006670, TESOP2006704, TESOP2006746, TESOP2006865, TESOP2007041, TESOP2007052, TESOP2007262, TESOP2007384, TESOP2007636, TESOP2007674, TESOP2007688, TESOP2007725, TESOP2007912, TESOP2007978, TESOP2008127, TESOP2008552,

TESOP2008556, TESOP2008563, TESOP2008820, TESOP2009121, TESOP2009201, TESOP2009247, TESOP2009555, TESTI1000018, TESTI1000019, TESTI1000025, TESTI1000029, TESTI1000030, TESTI1000042, TESTI1000045, TESTI1000051, TESTI1000055, TESTI1000064, TESTI1000070, TESTI1000085, TESTI1000088, TESTI1000093, TESTI1000094, TESTI1000096, TESTI1000101, TESTI1000109, TESTI1000125, TESTI1000127, TESTI1000128, TESTI1000131, TESTI1000138, TESTI1000142, TESTI1000157, TESTI1000163, TESTI1000168, TESTI1000169, TESTI1000179, TESTI1000183, TESTI1000191, TESTI1000257, TESTI1000266, TESTI1000319, TESTI1000330, TESTI1000348, TESTI1000390, TESTI1000391, TESTI1000459, TESTI1000491, TESTI1000545, TESTI2000006, TESTI2000018,

TESTI2000044, TESTI2000100, TESTI2000117, TESTI2000120, TESTI2000147, TESTI2000154, TESTI2000172, TESTI2000177, TESTI2000179, TESTI2000207, TESTI2000208, TESTI2000221, TESTI2000238, TESTI2000253, TESTI2000272, TESTI2000278, TESTI2000349, TESTI2000358, TESTI2000372, TESTI2000427, TESTI2000431, TESTI2000435, TESTI2000443, TESTI2000452, TESTI2000462, TESTI2000489, TESTI2000520, TESTI2000571, TESTI2000591, TESTI2000598, TESTI2000600, TESTI2000616, TESTI2000627, TESTI2000644, TESTI2000671, TESTI2000683, TESTI2000689, TESTI2000695, TESTI2000699, TESTI2000707, TESTI2000745, TESTI2000756, TESTI2000762, TESTI2000808, TESTI2000819, TESTI2000830, TESTI2000840, TESTI2000849, TESTI2000850, TESTI2000874,

TESTI2000883, TESTI2000909, TESTI2000951, TESTI2000981, TESTI2001008, TESTI2001049, TESTI2001082, TESTI2001120, TESTI2001134, TESTI2001141, TESTI2001180, TESTI2001208, TESTI2001229, TESTI2001236, TESTI2001237, TESTI2001269, TESTI2001306, TESTI2001313, TESTI2001330, TESTI2001339, TESTI2001345, TESTI2001352, TESTI2001364, TESTI2001412, TESTI2001498, TESTI2001511, TESTI2001512, TESTI2001518, TESTI2001556, TESTI2001593, TESTI2001613, TESTI2001621, TESTI2001661, TESTI2001665, TESTI2001671, TESTI2001697, TESTI2001725, TESTI2001758, TESTI2001766, TESTI2001792, TESTI2001793, TESTI2001795, TESTI2001812, TESTI2001815, TESTI2001823, TESTI2001826, TESTI2001827, TESTI2001852, TESTI2001869, TESTI2001879,

TESTI2001894, TESTI2001898, TESTI2001915, TESTI2001950, TESTI2001959, TESTI2001968, TESTI2001985, TESTI2001991, TESTI2002002, TESTI2002012, TESTI2002036, TESTI2002048, TESTI2002057, TESTI2002081, TESTI2002090, TESTI2002105, TESTI2002216, TESTI2002223, TESTI2002245, TESTI2002251, TESTI2002256, TESTI2002264, TESTI2002282, TESTI2002293, TESTI2002294, TESTI2002326, TESTI2002351, TESTI2002361, TESTI2002369, TESTI2002406, TESTI2002415, TESTI2002423, TESTI2002453, TESTI2002461, TESTI2002465, TESTI2002467, TESTI2002498, TESTI2002516, TESTI2002544, TESTI2002578, TESTI2002580, TESTI2002618, TESTI2002676, TESTI2002690, TESTI2002696, TESTI2002698, TESTI2002700, TESTI2002707, TESTI2002724, TESTI2002729,

TESTI2002752, TESTI2002753, TESTI2002789, TESTI2002802, TESTI2002806, TESTI2002840, TESTI2002866, TESTI2002910, TESTI2002912, TESTI2002928, TESTI2002937, TESTI2002965, TESTI2002993, TESTI2003020, TESTI2003031, TESTI2003037, TESTI2003044, TESTI2003059, TESTI2003061, TESTI2003071, TESTI2003074, TESTI2003089, TESTI2003104, TESTI2003109, TESTI2003117, TESTI2003127, TESTI2003130, TESTI2003131, TESTI2003139, TESTI2003141, TESTI2003152, TESTI2003181, TESTI2003193, TESTI2003196, TESTI2003228, TESTI2003236, TESTI2003255, TESTI2003258, TESTI2003277, TESTI2003280, TESTI2003299, TESTI2003311, TESTI2003321, TESTI2003325, TESTI2003327, TESTI2003330, TESTI2003347, TESTI2003354, TESTI2003356, TESTI2003372,

TESTI2003376, TESTI2003413, TESTI2003418, TESTI2003419, TESTI2003432, TESTI2003454, TESTI2003475, TESTI2003476, TESTI2003498, TESTI2003512, TESTI2003539, TESTI2003541, TESTI2003545, TESTI2003551, TESTI2003574, TESTI2003579, TESTI2003580, TESTI2003589, TESTI2003596, TESTI2003598, TESTI2003615, TESTI2003625, TESTI2003638, TESTI2003649, TESTI2003692, TESTI2003718, TESTI2003727, TESTI2003756, TESTI2003768, TESTI2003781, TESTI2003787, TESTI2003824, TESTI2003827, TESTI2003894, TESTI2003911, TESTI2003914, TESTI2003940, TESTI2003962, TESTI2003998, TESTI2004000, TESTI2004073, TESTI2004080, TESTI2004085, TESTI2004089, TESTI2004111, TESTI2004119, TESTI2004122, TESTI2004153, TESTI2004163, TESTI2004169,

TESTI2004207, TESTI2004215, TESTI2004227, TESTI2004229, TESTI2004243, TESTI2004255, TESTI2004287, TESTI2004313, TESTI2004318, TESTI2004322, TESTI2004391, TESTI2004399, TESTI2004423, TESTI2004431, TESTI2004432, TESTI2004452, TESTI2004459, TESTI2004490, TESTI2004539, TESTI2004552, TESTI2004560, TESTI2004574, TESTI2004601, TESTI2004611, TESTI2004649, TESTI2004654, TESTI2004675, TESTI2004687, TESTI2004689, TESTI2004700, TESTI2004706, TESTI2004712, TESTI2004734, TESTI2004783, TESTI2004791, TESTI2004793, TESTI2004860, TESTI2004906, TESTI2004920, TESTI2004936, TESTI2004941, TESTI2004971, TESTI2004982, TESTI2004993, TESTI2004994, TESTI2004999, TESTI2005017, TESTI2005025, TESTI2005040, TESTI2005060,

TESTI2005072, TESTI2005085, TESTI2005112, TESTI2005120, TESTI2005153, TESTI2005155, TESTI2005173, TESTI2005267, TESTI2005268, TESTI2005280, TESTI2005307, TESTI2005326, TESTI2005363, TESTI2005376, TESTI2005378, TESTI2005380, TESTI2005391, TESTI2005395, TESTI2005396, TESTI2005408, TESTI2005415, TESTI2005470, TESTI2005492, TESTI2005564, TESTI2005568, TESTI2005584, TESTI2005588, TESTI2005590, TESTI2005603, TESTI2005609, TESTI2005610, TESTI2005630, TESTI2005672, TESTI2005683, TESTI2005690, TESTI2005692, TESTI2005703, TESTI2005720, TESTI2005739, TESTI2005742, TESTI2005743, TESTI2005767, TESTI2005775, TESTI2005784, TESTI2005788, TESTI2005827, TESTI2005835, TESTI2005860, TESTI2005892, TESTI2005911,

TESTI2005933, TESTI2005937, TESTI2005948, TESTI2005979, TESTI2005981, TESTI2005982, TESTI2005984, TESTI2005986, TESTI2005987, TESTI2006007, TESTI2006008, TESTI2006015, TESTI2006016, TESTI2006035, TESTI2006040, TESTI2006041, TESTI2006045, TESTI2006067, TESTI2006083, TESTI2006097, TESTI2006109, TESTI2006120, TESTI2006125, TESTI2006179, TESTI2006209, TESTI2006210, TESTI2006212, TESTI2006218, TESTI2006238, TESTI2006255, TESTI2006258, TESTI2006278, TESTI2006333, TESTI2006341, TESTI2006360, TESTI2006383, TESTI2006409, TESTI2006425, TESTI2006437, TESTI2006453, TESTI2006457, TESTI2006460, TESTI2006465, TESTI2006483, TESTI2006499, TESTI2006523, TESTI2006543, TESTI2006565, TESTI2006572, TESTI2006615,

TESTI2006617, TESTI2006624, TESTI2006628, TESTI2006633, TESTI2006643, TESTI2006648, TESTI2006659, TESTI2006660, TESTI2006665, TESTI2006667, TESTI2006677, TESTI2006720, TESTI2006735, TESTI2006744, TESTI2006748, TESTI2006774, TESTI2006853, TESTI2006866, TESTI2006872, TESTI2006877, TESTI2006879, TESTI2006894, TESTI2006940, TESTI2006955, TESTI2006978, TESTI2006979, TESTI2006990, TESTI2007040, TESTI2007074, TESTI2007078, TESTI2007113, TESTI2007152, TESTI2007163, TESTI2007183, TESTI2007220, TESTI2007234, TESTI2007311, TESTI2007320, TESTI2007346, TESTI2007358, TESTI2007402, TESTI2007405, TESTI2007422, TESTI2007452, TESTI2007464, TESTI2007466, TESTI2007480, TESTI2007490, TESTI2007502, TESTI2007524,

TESTI2007533, TESTI2007576, TESTI2007613, TESTI2007657, TESTI2007685, TESTI2007692, TESTI2007749, TESTI2007750, TESTI2007808, TESTI2007814, TESTI2007829, TESTI2007864, TESTI2007867, TESTI2007872, TESTI2007906, TESTI2007922, TESTI2007951, TESTI2007972, TESTI2007973, TESTI2007998, TESTI2008014, TESTI2008020, TESTI2008032, TESTI2008033, TESTI2008046, TESTI2008081, TESTI2008099, TESTI2008139, TESTI2008144, TESTI2008161, TESTI2008189, TESTI2008204, TESTI2008219, TESTI2008233, TESTI2008234, TESTI2008237, TESTI2008278, TESTI2008320, TESTI2008331, TESTI2008343, TESTI2008387, TESTI2008389, TESTI2008394, TESTI2008405, TESTI2008420, TESTI2008425, TESTI2008440, TESTI2008567, TESTI2008595, TESTI2008621,

TESTI2008636, TESTI2008657, TESTI2008684, TESTI2008699, TESTI2008715, TESTI2008762, TESTI2008774, TESTI2008786, TESTI2008822, TESTI2008840, TESTI2008847, TESTI2008884, TESTI2008901, TESTI2008929, TESTI2008946, TESTI2008960, TESTI2009018, TESTI2009091, TESTI2009097, TESTI2009136, TESTI2009185, TESTI2009190, TESTI2009217, TESTI2009239, TESTI2009325, TESTI2009388, TESTI2009390, TESTI2009400, TESTI2009402, TESTI2009412, TESTI2009423, TESTI2009447, TESTI2009462, TESTI2009474, TESTI2009477, TESTI2009497, TESTI2009502, TESTI2009511, TESTI2009544, TESTI2009545, TESTI2009551, TESTI2009585, TESTI2009588, TESTI2009683, TESTI2009727, TESTI2009739, TESTI2009812, TESTI2009835, TESTI2009853, TESTI2009935,

TESTI2009977, TESTI2009987, TESTI2010009, TESTI2010154, TESTI2010180, TESTI2010239, TESTI2010280, TESTI2010354, TESTI2010396, TESTI2010400, TESTI2010409, TESTI2010572, TESTI2010587, TESTI2010591, TESTI2010617, TESTI2010682, TESTI2010724, TESTI2010732, TESTI2010734, TESTI2010755, TESTI2010793, TESTI2010806, TESTI2010833, TESTI2010872, TESTI2011009, TESTI2011020, TESTI2011028, TESTI2011033, TESTI2011254, TESTI2011286, TESTI2011294, TESTI2011314, TESTI2011315, TESTI2011394, TESTI2011407, TESTI2011448, TESTI2011480, TESTI2011549, TESTI2011575, TESTI2011605, TESTI2011612, TESTI2011665, TESTI2011680, TESTI2011712, TESTI2011750, TESTI2011840, TESTI2011846, TESTI2011915, TESTI2011928, TESTI2011971,

TESTI2012019, TESTI2012050, TESTI2012090, TESTI2012095, TESTI2012104, TESTI2012152, TESTI2012155, TESTI2012171, TESTI2012207, TESTI2012231, TESTI2012244, TESTI2012289, TESTI2012308, TESTI2012356, TESTI2012387, TESTI2012394, TESTI2012444, TESTI2012511, TESTI2012528, TESTI2012544, TESTI2012550, TESTI2012571, TESTI2012592, TESTI2012617, TESTI2012628, TESTI2012778, TESTI2012812, TESTI2012834, TESTI2012835, TESTI2012861, TESTI2012900, TESTI2012915, TESTI2012922, TESTI2013001, TESTI2013012, TESTI2013053, TESTI2013144, TESTI2013163, TESTI2013169, TESTI2013212, TESTI2013231, TESTI2013257, TESTI2013264, TESTI2013268, TESTI2013295, TESTI2013322, TESTI2013381, TESTI2013382, TESTI2013401, TESTI2013427,

TESTI2013468, TESTI2013497, TESTI2013545, TESTI2013546, TESTI2013566, TESTI2013601, TESTI2013604, TESTI2013607, TESTI2013610, TESTI2013691, TESTI2013699, TESTI2013737, TESTI2013767, TESTI2013832, TESTI2013873, TESTI2013889, TESTI2014007, TESTI2014036, TESTI2014097, TESTI2014160, TESTI2014248, TESTI2014254, TESTI2014269, TESTI2014274, TESTI2014318, TESTI2014324, TESTI2014339, TESTI2014362, TESTI2014385, TESTI2014415, TESTI2014439, TESTI2014474, TESTI2014505, TESTI2014577, TESTI2014578, TESTI2014716, TESTI2014780, TESTI2014800, TESTI2014843, TESTI2014979, TESTI2014988, TESTI2015042, TESTI2015092, TESTI2015105, TESTI2015180, TESTI2015246, TESTI2015249, TESTI2015327, TESTI2015331, TESTI2015335,

TESTI2015350, TESTI2015375, TESTI2015437, TESTI2015590, TESTI2015610, TESTI2015647, TESTI2015675, TESTI2015710, TESTI2015752, TESTI2015830, TESTI2015914, TESTI2015938, TESTI2015947, TESTI2015962, TESTI2015987, TESTI2016033, TESTI2016046, TESTI2016101, TESTI2016162, TESTI2016197, TESTI2016278, TESTI2016298, TESTI2016331, TESTI2016337, TESTI2016410, TESTI2016421, TESTI2016509, TESTI2016568, TESTI2016583, TESTI2016599, TESTI2016629, TESTI2016663, TESTI2016667, TESTI2016688, TESTI2016701, TESTI2016758, TESTI2016848, TESTI2016863, TESTI2016888, TESTI2016896, TESTI2016922, TESTI2016942, TESTI2016950, TESTI2016996, TESTI2017017, TESTI2017028, TESTI2017035, TESTI2017069, TESTI2017089, TESTI2017102,

TESTI2017107, TESTI2017113, TESTI2017125, TESTI2017256, TESTI2017305, TESTI2017311, TESTI2017396, TESTI2017507, TESTI2017537, TESTI2017582, TESTI2017584, TESTI2017645, TESTI2017727, TESTI2017740, TESTI2017775, TESTI2017816, TESTI2017832, TESTI2017923, TESTI2017932, TESTI2017951, TESTI2018014, TESTI2018031, TESTI2018035, TESTI2018060, TESTI2018083, TESTI2018178, TESTI2018221, TESTI2018276, TESTI2018290, TESTI2018335, TESTI2018337, TESTI2018368, TESTI2018421, TESTI2018428, TESTI2018475, TESTI2018482, TESTI2018521, TESTI2018565, TESTI2018581, TESTI2018611, TESTI2018687, TESTI2018697, TESTI2018838, TESTI2018867, TESTI2018941, TESTI2018957, TESTI2018974, TESTI2018975, TESTI2018997, TESTI2019042,

TESTI2019077, TESTI2019228, TESTI2019257, TESTI2019280, TESTI2019282, TESTI2019308, TESTI2019336, TESTI2019352, TESTI2019406, TESTI2019430, TESTI2019455, TESTI2019481, TESTI2019488, TESTI2019501, TESTI2019648, TESTI2019669, TESTI2019697, TESTI2019729, TESTI2019760, TESTI2019787, TESTI2019794, TESTI2019854, TESTI2019859, TESTI2019860, TESTI2019872, TESTI2019911, TESTI2019917, TESTI2019975, TESTI2019998, TESTI2020012, TESTI2020026, TESTI2020029, TESTI2020071, TESTI2020084, TESTI2020265, TESTI2020283, TESTI2020316, TESTI2020344, TESTI2020379, TESTI2020426, TESTI2020445, TESTI2020510, TESTI2020515, TESTI2020525, TESTI2020579, TESTI2020717, TESTI2020840, TESTI2020905, TESTI2020906, TESTI2020946,

TESTI2020956, TESTI2020981, TESTI2020999, TESTI2021003, TESTI2021038, TESTI2021057, TESTI2021077, TESTI2021112, TESTI2021114, TESTI2021116, TESTI2021118, TESTI2021122, TESTI2021138, TESTI2021297, TESTI2021315, TESTI2021447, TESTI2021463, TESTI2021531, TESTI2021599, TESTI2021637, TESTI2021654, TESTI2021667, TESTI2021736, TESTI2021785, TESTI2021819, TESTI2021851, TESTI2021895, TESTI2021911, TESTI2021939, TESTI2021999, TESTI2022010, TESTI2022023, TESTI2022065, TESTI2022086, TESTI2022179, TESTI2022203, TESTI2022246, TESTI2022323, TESTI2022338, TESTI2022400, TESTI2022462, TESTI2022495, TESTI2022623, TESTI2022649, TESTI2022652, TESTI2022738, TESTI2022798, TESTI2022812, TESTI2022814, TESTI2022874,

TESTI2022940, TESTI2022952, TESTI2023025, TESTI2023033, TESTI2023053, TESTI2023085, TESTI2023192, TESTI2023194, TESTI2023214, TESTI2023254, TESTI2023386, TESTI2023414, TESTI2023427, TESTI2023436, TESTI2023599, TESTI2023752, TESTI2023800, TESTI2023861, TESTI2023903, TESTI2023942, TESTI2023947, TESTI2023951, TESTI2023968, TESTI2024009, TESTI2024090, TESTI2024125, TESTI2024153, TESTI2024192, TESTI2024267, TESTI2024283, TESTI2024299, TESTI2024419, TESTI2024422, TESTI2024443, TESTI2024446, TESTI2024473, TESTI2024476, TESTI2024498, TESTI2024560, TESTI2024567, TESTI2024586, TESTI2024598, TESTI2024641, TESTI2024648, TESTI2024718, TESTI2024744, TESTI2024844, TESTI2024864, TESTI2024885, TESTI2024936,

TESTI2024978, TESTI2024999, TESTI2025006, TESTI2025013, TESTI2025022, TESTI2025063, TESTI2025117, TESTI2025144, TESTI2025161, TESTI2025174, TESTI2025229, TESTI2025269, TESTI2025365, TESTI2025403, TESTI2025409, TESTI2025422, TESTI2025448, TESTI2025454, TESTI2025486, TESTI2025546, TESTI2025582, TESTI2025609, TESTI2025656, TESTI2025757, TESTI2025791, TESTI2025846, TESTI2025872, TESTI2025879, TESTI2025911, TESTI2025920, TESTI2025924, TESTI2026014, TESTI2026024, TESTI2026064, TESTI2026077, TESTI2026104, TESTI2026116, TESTI2026134, TESTI2026168, TESTI2026196, TESTI2026215, TESTI2026218, TESTI2026233, TESTI2026291, TESTI2026294, TESTI2026401, TESTI2026403, TESTI2026453, TESTI2026461, TESTI2026491,

TESTI2026505, TESTI2026515, TESTI2026525, TESTI2026534, TESTI2026537, TESTI2026589, TESTI2026597, TESTI2026605, TESTI2026647, TESTI2026674, TESTI2026794, TESTI2026824, TESTI2026854, TESTI2026925, TESTI2026936, TESTI2026957, TESTI2027013, TESTI2027019, TESTI2027108, TESTI2027171, TESTI2027173, TESTI2027179, TESTI2027185, TESTI2027206, TESTI2027238, TESTI2027239, TESTI2027243, TESTI2027271, TESTI2027296, TESTI2027378, TESTI2027411, TESTI2027450, TESTI2027496, TESTI2027503, TESTI2027562, TESTI2027615, TESTI2027645, TESTI2027730, TESTI2027736, TESTI2027791, TESTI2027820, TESTI2027828, TESTI2027840, TESTI2027909, TESTI2028098, TESTI2028151, TESTI2028242, TESTI2028253, TESTI2028254, TESTI2028269,

TESTI2028290, TESTI2028296, TESTI2028384, TESTI2028426, TESTI2028488, TESTI2028523, TESTI2028583, TESTI2028613, TESTI2028659, TESTI2028662, TESTI2028670, TESTI2028705, TESTI2028776, TESTI2028811, TESTI2028891, TESTI2028985, TESTI2028997, TESTI2029131, TESTI2029162, TESTI2029196, TESTI2029201, TESTI2029227, TESTI2029244, TESTI2029249, TESTI2029252, TESTI2029264, TESTI2029470, TESTI2029672, TESTI2029785, TESTI2029880, TESTI2030136, TESTI2030322, TESTI2030336, TESTI2030342, TESTI2030519, TESTI2030554, TESTI2030556, TESTI2030728, TESTI2030754, TESTI2030770, TESTI2030860, TESTI2030901, TESTI2030917, TESTI2030930, TESTI2031006, TESTI2031007, TESTI2031060, TESTI2031129, TESTI2031356, TESTI2031418,

TESTI2031504, TESTI2031529, TESTI2031687, TESTI2031760, TESTI2031809, TESTI2031919, TESTI2031935, TESTI2031986, TESTI2032044, TESTI2032067, TESTI2032079, TESTI2032643, TESTI2032681, TESTI2032761, TESTI2032768, TESTI2032774, TESTI2032828, TESTI2033031, TESTI2033242, TESTI2033254, TESTI2033300, TESTI2033395, TESTI2033453, TESTI2033505, TESTI2033520, TESTI2033641, TESTI2033710, TESTI2033905, TESTI2034187, TESTI2034243, TESTI2034249, TESTI2034307, TESTI2034357, TESTI2034401, TESTI2034407, TESTI2034506, TESTI2034520, TESTI2034718, TESTI2034730, TESTI2034749, TESTI2034767, TESTI2034774, TESTI2034777, TESTI2034785, TESTI2034799, TESTI2034847, TESTI2034913, TESTI2034916, TESTI2034931, TESTI2034953,

TESTI2034997, TESTI2035003, TESTI2035078, TESTI2035084, TESTI2035107, TESTI2035121, TESTI2035124, TESTI2035183, TESTI2035236, TESTI2035262, TESTI2035309, TESTI2035688, TESTI2035775, TESTI2035793, TESTI2035898, TESTI2035962, TESTI2035981, TESTI2035997, TESTI2036114, TESTI2036129, TESTI2036285, TESTI2036288, TESTI2036513, TESTI2036589, TESTI2036650, TESTI2036684, TESTI2036691, TESTI2036736, TESTI2036822, TESTI2036833, TESTI2036922, TESTI2036951, TESTI2036965, TESTI2037002, TESTI2037055, TESTI2037081, TESTI2037085, TESTI2037103, TESTI2037106, TESTI2037111, TESTI2037209, TESTI2037255, TESTI2037359, TESTI2037375, TESTI2037382, TESTI2037534, TESTI2037569, TESTI2037572, TESTI2037643, TESTI2037657,

TESTI2037673, TESTI2037723, TESTI2037819, TESTI2037830, TESTI2037845, TESTI2037877, TESTI2037976, TESTI2038048, TESTI2038065, TESTI2038104, TESTI2038275, TESTI2038388, TESTI2038573, TESTI2038596, TESTI2038623, TESTI2038644, TESTI2038733, TESTI2038858, TESTI2038958, TESTI2039025, TESTI2039026, TESTI2039038, TESTI2039041, TESTI2039060, TESTI2039113, TESTI2039121, TESTI2039209, TESTI2039225, TESTI2039227, TESTI2039276, TESTI2039342, TESTI2039353, TESTI2039544, TESTI2039613, TESTI2039707, TESTI2039732, TESTI2039738, TESTI2039776, TESTI2039867, TESTI2039908, TESTI2040018, TESTI2040094, TESTI2040102, TESTI2040128, TESTI2040143, TESTI2040297, TESTI2040372, TESTI2040377, TESTI2040424, TESTI2040642,

TESTI2040815, TESTI2040897, TESTI2040944, TESTI2040964, TESTI2040983, TESTI2040989, TESTI2041330, TESTI2041362, TESTI2041517, TESTI2041564, TESTI2041664, TESTI2041730, TESTI2041734, TESTI2041778, TESTI2041800, TESTI2041947, TESTI2041956, TESTI2041976, TESTI2042062, TESTI2042149, TESTI2042264, TESTI2042295, TESTI2042302, TESTI2042326, TESTI2042400, TESTI2042450, TESTI2042473, TESTI2042507, TESTI2042508, TESTI2042750, TESTI2042783, TESTI2042806, TESTI2042928, TESTI2042946, TESTI2042958, TESTI2043275, TESTI2043277, TESTI2043282, TESTI2043313, TESTI2043477, TESTI2043585, TESTI2043758, TESTI2043817, TESTI2043857, TESTI2043866, TESTI2043869, TESTI2044064, TESTI2044118, TESTI2044194, TESTI2044276,

TESTI2044306, TESTI2044309, TESTI2044413, TESTI2044418, TESTI2044754, TESTI2044796, TESTI2044833, TESTI2044892, TESTI2044920, TESTI2044968, TESTI2045139, TESTI2045155, TESTI2045171, TESTI2045199, TESTI2045353, TESTI2045509, TESTI2045562, TESTI2045611, TESTI2045819, TESTI2045850, TESTI2045920, TESTI2045964, TESTI2045983, TESTI2046100, TESTI2046188, TESTI2046347, TESTI2046352, TESTI2046358, TESTI2046439, TESTI2046535, TESTI2046536, TESTI2046552, TESTI2046569, TESTI2046686, TESTI2046721, TESTI2046732, TESTI2046786, TESTI2046797, TESTI2046863, TESTI2046872, TESTI2047071, TESTI2047141, TESTI2047147, TESTI2047153, TESTI2047212, TESTI2047342, TESTI2047383, TESTI2047792, TESTI2047801, TESTI2047818,

TESTI2047824, TESTI2047885, TESTI2047930, TESTI2048105, TESTI2048109, TESTI2048465, TESTI2048603, TESTI2048898, TESTI2049041, TESTI2049062, TESTI2049206, TESTI2049246, TESTI2049277, TESTI2049422, TESTI2049452, TESTI2049469, TESTI2049553, TESTI2049576, TESTI2049857, TESTI2049956, TESTI2050137, TESTI2050681, TESTI2050780, TESTI2050987, TESTI2051148, TESTI2051177, TESTI2051279, TESTI2051488, TESTI2051543, TESTI2051627, TESTI2051742, TESTI2051767, TESTI2051791, TESTI2051806, TESTI2051867, TESTI2051873, TESTI2052110, TESTI2052202, TESTI2052211, TESTI2052256, TESTI2052288, TESTI2052525, TESTI2052670, TESTI2052693, TESTI2052698, TESTI2052799, TESTI2052822, TESTI2052985, TESTI2053151, TESTI2053242,

TESTI2053399, TESTI2053526, TESTI2053561, TESTI2053621, TESTI2053667, TESTI3000002, TESTI3000005, TESTI4000068, TESTI4000079, TESTI4000093, TESTI4000134, TESTI4000137, TESTI4000153, TESTI4000155, TESTI4000157, TESTI4000183, TESTI4000214, TESTI4000215, TESTI4000228, TESTI4000250, TESTI4000288, TESTI4000319, TESTI4000349, TESTI4000370, TESTI4000394, TESTI4000434, TESTI4000462, TESTI4000530, TESTI4000534, TESTI4000598, TESTI4000600, TESTI4000621, TESTI4000703, TESTI4000724, TESTI4000957, TESTI4000970, TESTI4001036, TESTI4001037, TESTI4001060, TESTI4001106, TESTI4001148, TESTI4001176, TESTI4001195, TESTI4001201, TESTI4001206, TESTI4001348, TESTI4001441, TESTI4001473, TESTI4001517, TESTI4001527,

TESTI4001561, TESTI4001569, TESTI4001604, TESTI4001665, TESTI4001679, TESTI4001795, TESTI4001923, TESTI4001925, TESTI4001984, TESTI4002003, TESTI4002141, TESTI4002202, TESTI4002290, TESTI4002319, TESTI4002491, TESTI4002520, TESTI4002552, TESTI4002647, TESTI4002703, TESTI4002754, TESTI4002868, TESTI4002878, TESTI4002889, TESTI4002988, TESTI4003150, TESTI4003179, TESTI4003279, TESTI4003319, TESTI4003404, TESTI4003565, TESTI4003574, TESTI4003579, TESTI4003602, TESTI4003733, TESTI4003796, TESTI4004003, TESTI4004031, TESTI4004200, TESTI4004210, TESTI4004539, TESTI4004626, TESTI4004653, TESTI4004695, TESTI4004722, TESTI4004887, TESTI4004917, TESTI4005015, TESTI4005039, TESTI4005133, TESTI4005158,

TESTI4005317, TESTI4005322, TESTI4005470, TESTI4005500, TESTI4005543, TESTI4005628, TESTI4005801, TESTI4005805, TESTI4005857, TESTI4005961, TESTI4006053, TESTI4006079, TESTI4006112, TESTI4006137, TESTI4006148, TESTI4006219, TESTI4006234, TESTI4006308, TESTI4006326, TESTI4006393, TESTI4006407, TESTI4006412, TESTI4006420, TESTI4006441, TESTI4006473, TESTI4006539, TESTI4006546, TESTI4006567, TESTI4006704, TESTI4006728, TESTI4006802, TESTI4006819, TESTI4007064, TESTI4007163, TESTI4007176, TESTI4007203, TESTI4007239, TESTI4007369, TESTI4007373, TESTI4007382, TESTI4007404, TESTI4007489, TESTI4007565, TESTI4007671, TESTI4007775, TESTI4007778, TESTI4007799, TESTI4007810, TESTI4007816, TESTI4007965,

TESTI4008007, TESTI4008018, TESTI4008050, TESTI4008058, TESTI4008086, TESTI4008097, TESTI4008176, TESTI4008209, TESTI4008219, TESTI4008250, TESTI4008302, TESTI4008305, TESTI4008401, TESTI4008417, TESTI4008429, TESTI4008573, TESTI4008816, TESTI4008840, TESTI4008935, TESTI4008993, TESTI4009022, TESTI4009028, TESTI4009034, TESTI4009123, TESTI4009160, TESTI4009215, TESTI4009283, TESTI4009306, TESTI4009374, TESTI4009406, TESTI4009454, TESTI4009457, TESTI4009501, TESTI4009563, TESTI4009608, TESTI4009638, TESTI4009752, TESTI4009881, TESTI4009886, TESTI4009928, TESTI4010076, TESTI4010082, TESTI4010095, TESTI4010211, TESTI4010377, TESTI4010475, TESTI4010544, TESTI4010713, TESTI4010721, TESTI4010789,

TESTI4010817, TESTI4010831, TESTI4010851, TESTI4010902, TESTI4010928, TESTI4010979, TESTI4011070, TESTI4011072, TESTI4011084, TESTI4011118, TESTI4011246, TESTI4011484, TESTI4011505, TESTI4011532, TESTI4011616, TESTI4011744, TESTI4011745, TESTI4011829, TESTI4011926, TESTI4011956, TESTI4012010, TESTI4012086, TESTI4012258, TESTI4012293, TESTI4012329, TESTI4012382, TESTI4012406, TESTI4012448, TESTI4012451, TESTI4012505, TESTI4012556, TESTI4012623, TESTI4012646, TESTI4012679, TESTI4012911, TESTI4012956, TESTI4012960, TESTI4013365, TESTI4013369, TESTI4013410, TESTI4013474, TESTI4013602, TESTI4013667, TESTI4013685, TESTI4013735, TESTI4013742, TESTI4013774, TESTI4013817, TESTI4013830, TESTI4013852,

TESTI4013924, TESTI4013960, TESTI4013962, TESTI4014175, TESTI4014265, TESTI4014276, TESTI4014306, TESTI4014392, TESTI4014415, TESTI4014445, TESTI4014553, TESTI4014661, TESTI4014694, TESTI4014801, TESTI4014818, TESTI4014891, TESTI4014908, TESTI4014924, TESTI4014932, TESTI4014977, TESTI4015012, TESTI4015129, TESTI4015145, TESTI4015204, TESTI4015263, TESTI4015293, TESTI4015299, TESTI4015339, TESTI4015398, TESTI4015442, TESTI4015471, TESTI4015477, TESTI4015600, TESTI4015646, TESTI4015681, TESTI4015688, TESTI4015790, TESTI4016110, TESTI4016238, TESTI4016551, TESTI4016812, TESTI4016822, TESTI4016865, TESTI4016882, TESTI4016925, TESTI4017001, TESTI4017116, TESTI4017137, TESTI4017229, TESTI4017254,

TESTI4017269, TESTI4017380, TESTI4017382, TESTI4017543, TESTI4017575, TESTI4017647, TESTI4017714, TESTI4017763, TESTI4017848, TESTI4017854, TESTI4017901, TESTI4017961, TESTI4017984, TESTI4018101, TESTI4018152, TESTI4018208, TESTI4018382, TESTI4018436, TESTI4018506, TESTI4018555, TESTI4018751, TESTI4018806, TESTI4018835, TESTI4018859, TESTI4018881, TESTI4018938, TESTI4019140, TESTI4019149, TESTI4019299, TESTI4019417, TESTI4019440, TESTI4019566, TESTI4019657, TESTI4019756, TESTI4019843, TESTI4020092, TESTI4020102, TESTI4020342, TESTI4020460, TESTI4020596, TESTI4020806, TESTI4020819, TESTI4020920, TESTI4021129, TESTI4021197, TESTI4021294, TESTI4021377, TESTI4021456, TESTI4021491, TESTI4021569,

TESTI4021713, TESTI4021821, TESTI4022158, TESTI4022577, TESTI4022716, TESTI4022873, TESTI4023096, TESTI4023172, TESTI4023546, TESTI4023555, TESTI4023654, TESTI4023722, TESTI4023762, TESTI4023942, TESTI4024096, TESTI4024240, TESTI4024294, TESTI4024344, TESTI4024387, TESTI4024420, TESTI4024494, TESTI4024740, TESTI4024874, TESTI4024890, TESTI4024907, TESTI4024930, TESTI4025062, TESTI4025268, TESTI4025401, TESTI4025494, TESTI4025547, TESTI4025731, TESTI4025797, TESTI4025865, TESTI4025908, TESTI4025920, TESTI4026079, TESTI4026080, TESTI4026192, TESTI4026207, TESTI4026295, TESTI4026390, TESTI4026456, TESTI4026510, TESTI4026524, TESTI4026680, TESTI4026700, TESTI4026762, TESTI4026785, TESTI4027139,

TESTI4027170, TESTI4027262, TESTI4027516, TESTI4027557, TESTI4027660, TESTI4027821, TESTI4027969, TESTI4028042, TESTI4028059, TESTI4028062, TESTI4028182, TESTI4028429, TESTI4028612, TESTI4028692, TESTI4028809, TESTI4028823, TESTI4028880, TESTI4028904, TESTI4028938, TESTI4028958, TESTI4028983, TESTI4029023, TESTI4029348, TESTI4029370, TESTI4029528, TESTI4029651, TESTI4029676, TESTI4029690, TESTI4029731, TESTI4029743, TESTI4030069, TESTI4030159, TESTI4030319, TESTI4030505, TESTI4030603, TESTI4030669, TESTI4030673, TESTI4030864, TESTI4031066, TESTI4031173, TESTI4031335, TESTI4031745, TESTI4031818, TESTI4032090, TESTI4032112, TESTI4032128, TESTI4032270, TESTI4032375, TESTI4032834, TESTI4032856,

TESTI4032895, TESTI4032913, TESTI4033146, TESTI4033177, TESTI4033433, TESTI4033690, TESTI4034172, TESTI4034212, TESTI4034432, TESTI4034495, TESTI4034632, TESTI4034633, TESTI4034912, TESTI4034973, TESTI4035063, TESTI4035065, TESTI4035498, TESTI4035508, TESTI4035581, TESTI4035602, TESTI4035637, TESTI4035649, TESTI4035770, TESTI4035872, TESTI4035895, TESTI4035898, TESTI4035989, TESTI4036012, TESTI4036042, TESTI4036048, TESTI4036315, TESTI4036319, TESTI4036449, TESTI4036767, TESTI4036909, TESTI4037066, TESTI4037188, TESTI4037228, TESTI4037244, TESTI4037727, TESTI4037949, TESTI4038047, TESTI4038099, TESTI4038156, TESTI4038223, TESTI4038258, TESTI4038339, TESTI4038492, TESTI4038721, TESTI4038758,

TESTI4038779, TESTI4038818, TESTI4039038, TESTI4039086, TESTI4039451, TESTI4039575, TESTI4039659, TESTI4039744, TESTI4039904, TESTI4040197, TESTI4040363, TESTI4040535, TESTI4040559, TESTI4040598, TESTI4040800, TESTI4040804, TESTI4040939, TESTI4040956, TESTI4041049, TESTI4041053, TESTI4041086, TESTI4041099, TESTI4041143, TESTI4041482, TESTI4041519, TESTI4041624, TESTI4041629, TESTI4041832, TESTI4041844, TESTI4041903, TESTI4041954, TESTI4041984, TESTI4041985, TESTI4041986, TESTI4042098, TESTI4042420, TESTI4042440, TESTI4042444, TESTI4042711, TESTI4042846, TESTI4043067, TESTI4043129, TESTI4043140, TESTI4043166, TESTI4043203, TESTI4043223, TESTI4043371, TESTI4043378, TESTI4043512, TESTI4043551,

TESTI4043710, TESTI4043947, TESTI4044035, TESTI4044076, TESTI4044084, TESTI4044123, TESTI4044186, TESTI4044234, TESTI4044291, TESTI4044296, TESTI4044300, TESTI4044682, TESTI4044704, TESTI4044770, TESTI4045168, TESTI4045312, TESTI4045330, TESTI4045470, TESTI4045701, TESTI4045908, TESTI4045955, TESTI4045982, TESTI4046073, TESTI4046090, TESTI4046240, TESTI4046245, TESTI4046253, TESTI4046282, TESTI4046328, TESTI4046450, TESTI4046457, TESTI4046487, TESTI4046502, TESTI4046523, TESTI4046819, TESTI4046873, TESTI4046884, TESTI4046896, TESTI4046903, TESTI4046962, TESTI4047069, TESTI4047119, TESTI4047305, TESTI4047328, TESTI4047437, TESTI4047569, TESTI4047746, TESTI4047808, THYMU1000010, THYMU1000017,

THYMU1000025, THYMU1000029, THYMU1000032, THYMU1000033, THYMU1000060, THYMU1000074, THYMU1000098, THYMU1000105, THYMU1000136, THYMU1000144, THYMU1000158, THYMU1000176, THYMU1000179, THYMU1000191, THYMU1000198, THYMU1000217, THYMU1000240, THYMU1000242, THYMU1000291, THYMU1000318, THYMU1000329, THYMU1000331, THYMU1000336, THYMU1000366, THYMU1000382, THYMU1000393, THYMU1000399, THYMU1000426, THYMU1000440, THYMU1000459, THYMU1000473, THYMU1000490, THYMU1000491, THYMU1000496, THYMU1000499, THYMU1000527, THYMU1000532, THYMU1000536, THYMU1000554, THYMU1000558, THYMU1000576, THYMU1000600, THYMU1000677, THYMU1000692, THYMU2000022, THYMU2000032, THYMU2000057, THYMU2000120, THYMU2000138, THYMU2000140,

THYMU2000155, THYMU2000162, THYMU2000171, THYMU2000176, THYMU2000179, THYMU2000231, THYMU2000236, THYMU2000276, THYMU2000280, THYMU2000300, THYMU2000317, THYMU2000368, THYMU2000369, THYMU2000418, THYMU2000440, THYMU2000489, THYMU2000492, THYMU2000519, THYMU2000570, THYMU2000602, THYMU2000660, THYMU2000671, THYMU2000672, THYMU2000702, THYMU2000767, THYMU2000775, THYMU2000800, THYMU2000879, THYMU2000932, THYMU2000946, THYMU2000950, THYMU2000971, THYMU2000982, THYMU2000990, THYMU2001018, THYMU2001053, THYMU2001071, THYMU2001090, THYMU2001139, THYMU2001195, THYMU2001202, THYMU2001203, THYMU2001256, THYMU2001270, THYMU2001291, THYMU2001314, THYMU2001325, THYMU2001357, THYMU2001381, THYMU2001443,

THYMU2001515, THYMU2001521, THYMU2001556, THYMU2001622, THYMU2001727, THYMU2001819, THYMU2001825, THYMU2001839, THYMU2001868, THYMU2001898, THYMU2001900, THYMU2001916, THYMU2001926, THYMU2001989, THYMU2002023, THYMU2002037, THYMU2002080, THYMU2002109, THYMU2002147, THYMU2002154, THYMU2002157, THYMU2002162, THYMU2002177, THYMU2002203, THYMU2002229, THYMU2002242, THYMU2002338, THYMU2002356, THYMU2002376, THYMU2002384, THYMU2002450, THYMU2002490, THYMU2002548, THYMU2002695, THYMU2002745, THYMU2002756, THYMU2002789, THYMU2002792, THYMU2002841, THYMU2002852, THYMU2002910, THYMU2002950, THYMU2002983, THYMU2003069, THYMU2003107, THYMU2003124, THYMU2003133, THYMU2003166, THYMU2003232, THYMU2003242,

THYMU2003249, THYMU2003270, THYMU2003282, THYMU2003287, THYMU2003336, THYMU2003366, THYMU2003397, THYMU2003440, THYMU2003470, THYMU2003479, THYMU2003499, THYMU2003542, THYMU2003632, THYMU2003650, THYMU2003700, THYMU2003760, THYMU2003803, THYMU2003855, THYMU2003907, THYMU2003932, THYMU2004003, THYMU2004008, THYMU2004042, THYMU2004108, THYMU2004111, THYMU2004123, THYMU2004139, THYMU2004169, THYMU2004284, THYMU2004320, THYMU2004336, THYMU2004344, THYMU2004356, THYMU2004452, THYMU2004508, THYMU2004512, THYMU2004635, THYMU2004639, THYMU2004668, THYMU2004677, THYMU2004688, THYMU2004693, THYMU2004733, THYMU2004835, THYMU2004843, THYMU2004906, THYMU2004916, THYMU2004986, THYMU2005001, THYMU2005003,

THYMU2005046, THYMU2005134, THYMU2005156, THYMU2005190, THYMU2005225, THYMU2005240, THYMU2005246, THYMU2005270, THYMU2005283, THYMU2005303, THYMU2005321, THYMU2005356, THYMU2005369, THYMU2005439, THYMU2005480, THYMU2005482, THYMU2005507, THYMU2005545, THYMU2005574, THYMU2005576, THYMU2005748, THYMU2005759, THYMU2005807, THYMU2005815, THYMU2005855, THYMU2005881, THYMU2005948, THYMU2005980, THYMU2005997, THYMU2006001, THYMU2006048, THYMU2006098, THYMU2006160, THYMU2006170, THYMU2006252, THYMU2006261, THYMU2006277, THYMU2006303, THYMU2006324, THYMU2006350, THYMU2006357, THYMU2006365, THYMU2006420, THYMU2006468, THYMU2006610, THYMU2006666, THYMU2006712, THYMU2006913, THYMU2006946, THYMU2006965, THYMU2007025, THYMU2007036, THYMU2007060, THYMU2007065, THYMU2007095, THYMU2007112, THYMU2007146, THYMU2007179, THYMU2007258, THYMU2007307, THYMU2007308, THYMU2007339, THYMU2007350, THYMU2007384, THYMU2007493, THYMU2007521, THYMU2007528, THYMU2007530, THYMU2007532, THYMU2007561, THYMU2007571, THYMU207635, THYMU2007658, THYMU2007667, THYMU2007694, THYMU2007725, THYMU2007729, THYMU2007802, THYMU2007824, THYMU2007854, THYMU2007952, THYMU2007969, THYMU2008035, THYMU2008036, THYMU2008049, THYMU2008072, THYMU2008111, THYMU2008145, THYMU2008149, THYMU2008185, THYMU2008191, THYMU2008207, THYMU2008226, THYMU2008282, THYMU2008321, THYMU2008339, THYMU2008350, THYMU2008351, THYMU2008383, THYMU2008452,

THYMU2008643, THYMU2008691, THYMU2008714, THYMU2008727, THYMU2008781, THYMU2008799, THYMU2008876, THYMU2008910, THYMU2008917, THYMU2008928, THYMU2008990, THYMU2008994, THYMU2009023, THYMU2009046, THYMU2009101, THYMU2009104, THYMU2009134, THYMU2009157, THYMU2009181, THYMU2009186, THYMU2009338, THYMU2009425, THYMU2009516, THYMU2009526, THYMU2009546, THYMU2009592, THYMU2009596, THYMU2009792, THYMU2009835, THYMU2009906, THYMU2009948, THYMU2010030, THYMU2010041, THYMU2010046, THYMU2010059, THYMU2010082, THYMU2010161, THYMU2010192, THYMU2010519, THYMU2010637, THYMU2010671, THYMU2010699, THYMU2010705, THYMU2010779, THYMU2010816, THYMU2010831, THYMU2010848, THYMU2011053, THYMU2011063, THYMU2011072,

THYMU2011096, THYMU2011118, THYMU2011142, THYMU2011183, THYMU2011257, THYMU2011259, THYMU2011295, THYMU2011308, THYMU2011390, THYMU2011431, THYMU2011447, THYMU2011482, THYMU2011538, THYMU2011548, THYMU2011551, THYMU2011564, THYMU2011573, THYMU2011585, THYMU2011621, THYMU2011666, THYMU2011736, THYMU2011785, THYMU2011806, THYMU2011852, THYMU2011875, THYMU2011881, THYMU2011937, THYMU2011939, THYMU2012002, THYMU2012073, THYMU2012104, THYMU2012113, THYMU2012273, THYMU2012401, THYMU2012492, THYMU2012507, THYMU2012565, THYMU2012574, THYMU2012625, THYMU2012631, THYMU2012665, THYMU2012690, THYMU2012701, THYMU2012807, THYMU2012826, THYMU2012882, THYMU2012891, THYMU2012902, THYMU2012919, THYMU2013023,

THYMU2013047, THYMU2013051, THYMU2013089, THYMU2013136, THYMU2013170, THYMU2013364, THYMU2013386, THYMU2013426, THYMU2013578, THYMU2013648, THYMU2013705, THYMU2013757, THYMU2013779, THYMU2013863, THYMU2013950, THYMU2013981, THYMU2014067, THYMU2014068, THYMU2014167, THYMU2014183, THYMU2014204, THYMU2014297, THYMU2014323, THYMU2014327, THYMU2014353, THYMU2014492, THYMU2014500, THYMU2014599, THYMU2014762, THYMU2014777, THYMU2014801, THYMU2014901, THYMU2014923, THYMU2015019, THYMU2015161, THYMU2015316, THYMU2015439, THYMU2015441, THYMU2015479, THYMU2015762, THYMU2015775, THYMU2015825, THYMU2015918, THYMU2015943, THYMU2015984, THYMU2016113, THYMU2016164, THYMU2016204, THYMU2016219, THYMU2016360,

THYMU2016496, THYMU2016523, THYMU2016840, THYMU2016968, THYMU2017008, THYMU2017010, THYMU2017023, THYMU2017085, THYMU2017099, THYMU2017158, THYMU2017162, THYMU2017215, THYMU2017362, THYMU2017366, THYMU2017398, THYMU2017449, THYMU2017479, THYMU2017522, THYMU2017526, THYMU2017601, THYMU2017675, THYMU2017707, THYMU2017831, THYMU2017844, THYMU2017878, THYMU2017948, THYMU2018028, THYMU2018071, THYMU2018126, THYMU2018189, THYMU2018197, THYMU2018384, THYMU2018455, THYMU2018547, THYMU2018565, THYMU2018639, THYMU2018673, THYMU2018721, THYMU2018772, THYMU2018819, THYMU2018841, THYMU2018997, THYMU2019054, THYMU2019197, THYMU2019210, THYMU2019364, THYMU2019436, THYMU2019442, THYMU2019587, THYMU2019599,

THYMU2019686, THYMU2019813, THYMU2020198, THYMU2020289, THYMU2020416, THYMU2020491, THYMU2020499, THYMU2020560, THYMU2020830, THYMU2020883, THYMU2020959, THYMU2021361, THYMU2021509, THYMU2021597, THYMU2021714, THYMU2022213, THYMU2022289, THYMU2022660, THYMU2022854, THYMU2022922, THYMU2023209, THYMU2023264, THYMU2023576, THYMU2023711, THYMU2023900, THYMU2023943, THYMU2023967, THYMU2024121, THYMU2024185, THYMU2024207, THYMU2024616, THYMU2024684, THYMU2024748, THYMU2025042, THYMU2025189, THYMU2025325, THYMU2025406, THYMU2025557, THYMU2025572, THYMU2025707, THYMU2025849, THYMU2025909, THYMU2025939, THYMU2026182, THYMU2026350, THYMU2026530, THYMU2026531, THYMU2026835, THYMU2026904, THYMU2027053,

THYMU2027125, THYMU2027168, THYMU2027249, THYMU2027282, THYMU2027497, THYMU2027695, THYMU2027734, THYMU2027894, THYMU2027975, THYMU2027996, THYMU2028041, THYMU2028368, THYMU2028379, THYMU2028394, THYMU2028412, THYMU2028629, THYMU2028632, THYMU2028724, THYMU2028739, THYMU2028942, THYMU2028978, THYMU2029134, THYMU2029578, THYMU2029676, THYMU2029688, THYMU2029788, THYMU2030068, THYMU2030226, THYMU2030264, THYMU2030462, THYMU2030543, THYMU2030637, THYMU2030655, THYMU2030796, THYMU2030912, THYMU2030982, THYMU2031046, THYMU2031139, THYMU2031218, THYMU2031249, THYMU2031258, THYMU2031341, THYMU2031368, THYMU2031579, THYMU2031847, THYMU2031994, THYMU2032014, THYMU2032035, THYMU2032080, THYMU2032150,

THYMU2032155, THYMU2032358, THYMU2032437, THYMU2032601, THYMU2032655, THYMU2032696, THYMU2032715, THYMU2032732, THYMU2032825, THYMU2032976, THYMU2033053, THYMU2033059, THYMU2033079, THYMU2033085, THYMU2033104, THYMU2033227, THYMU2033263, THYMU2033308, THYMU2033401, THYMU2033583, THYMU2033755, THYMU2033787, THYMU2033816, THYMU2034182, THYMU2034279, THYMU2034314, THYMU2034338, THYMU2034374, THYMU2034647, THYMU2035064, THYMU2035078, THYMU2035101, THYMU2035319, THYMU2035321, THYMU2035388, THYMU2035400, THYMU2035554, THYMU2035710, THYMU2036058, THYMU2036105, THYMU2036207, THYMU2036215, THYMU2036252, THYMU2036265, THYMU2036366, THYMU2036459, THYMU2036604, THYMU2036653, THYMU2037081, THYMU2037208,

THYMU2037226, THYMU2037233, THYMU2037234, THYMU2037348, THYMU2037406, THYMU2037965, THYMU2038058, THYMU2038189, THYMU2038199, THYMU2038220, THYMU2038301, THYMU2038369, THYMU2038389, THYMU2038615, THYMU2038636, THYMU2038739, THYMU2038772, THYMU2038797, THYMU2038906, THYMU2039305, THYMU2039315, THYMU2039334, THYMU2039350, THYMU2039411, THYMU2039634, THYMU2039719, THYMU2039780, THYMU2039788, THYMU2039989, THYMU2040029, THYMU2040110, THYMU2040114, THYMU2040140, THYMU2040412, THYMU2040427, THYMU2040824, THYMU2040925, THYMU2041007, THYMU2041015, THYMU2041252, THYMU3000028, THYMU3000036, THYMU3000037, THYMU3000082, THYMU3000194, THYMU3000224, THYMU3000269, THYMU3000306, THYMU3000360, THYMU3000390,

THYMU3000420, THYMU3000490, THYMU3000504, THYMU3000542, THYMU3000655, THYMU3000776, THYMU3000826, THYMU3000841, THYMU3001077, THYMU3001082, THYMU3001083, THYMU3001133, THYMU3001151, THYMU3001379, THYMU3001593, THYMU3001883, THYMU3001939, THYMU3001991, THYMU3002452, THYMU3002661, THYMU3003007, THYMU3003092, THYMU3003212, THYMU3003309, THYMU3003350, THYMU3003403, THYMU3003565, THYMU3003763, THYMU3003865, THYMU3003958, THYMU3003965, THYMU3004157, THYMU3004628, THYMU3004632, THYMU3004835, THYMU3004866, THYMU3005050, THYMU3005378, THYMU3005407, THYMU3005415, THYMU3005427, THYMU3005482, THYMU3005491, THYMU3005629, THYMU3005696, THYMU3006118, THYMU3006132, THYMU3006168, THYMU3006172, THYMU3006371,

THYMU3006485, THYMU3006640, THYMU3006767, THYMU3006811, THYMU3006963, THYMU3007137, THYMU3007308, THYMU3007368, THYMU3007423, THYMU3007559, THYMU3007845, THYMU3008060, THYMU3008105, THYMU3008136, THYMU3008171, THYMU3008216, THYMU3008436, THYMU3008672, THYMU3008883, THYMU3008935, THYMU3009006, THYMU3009255, THYMU3009391, THYMU3009643, THYMU3009755, THYMU3010247, THYMU3011012, THYMU3011081, THYMU3011244, THYMU3011360, THYMU3011534, THYMU3011543, THYMU3011556, THYMU3011717, THYMU3011827, THYMU3012402, THYMU3012907, THYMU3012983, THYMU3013114, THYMU3013197, THYMU3013241, THYMU3013470, THYMU3013785, THYMU3013897, THYMU3014038, THYMU3014173, THYMU3014372, THYMU3014555, THYMU3014620, THYMU3014701,

THYMU3015042, THYMU3015457, THYMU3015571, THYMU3015592, THYMU3015625, THYMU3015647, THYMU3015759, THYMU3015962, THYMU3016022, THYMU3016518, THYMU3016797, THYMU3016822, THYMU3016850, THYMU3017562, THYMU3017688, THYMU3017689, THYMU3017761, THYMU3018151, THYMU3018896, THYMU3019095, THYMU3019476, THYMU3019605, THYMU3019916, THYMU3020221, THYMU3020595, THYMU3020713, THYMU3020856, THYMU3020869, THYMU3020970, THYMU3021404, THYMU3021755, THYMU3021900, THYMU3022211, THYMU3022434, THYMU3022528, THYMU3022668, THYMU3022982, THYMU3023107, THYMU3023211, THYMU3023216, THYMU3023394, THYMU3023400, THYMU3023797, THYMU3024164, THYMU3024339, THYMU3024602, THYMU3024879, THYMU3025118, THYMU3025313, THYMU3025642,

THYMU3025683, THYMU3025772, THYMU3025865, THYMU3026000, THYMU3026479, THYMU3026532, THYMU3026783, THYMU3026869, THYMU3027251, THYMU3027540, THYMU3027655, THYMU3027671, THYMU3028075, THYMU3028461, THYMU3028702, THYMU3029188, THYMU3029318, THYMU3029421, THYMU3029719, THYMU3029774, THYMU3029832, THYMU3030072, THYMU3030231, THYMU3030706, THYMU3030710, THYMU3030752, THYMU3031146, THYMU3031402, THYMU3031610, THYMU3031612, THYMU3031868, THYMU3031878, THYMU3032032, THYMU3032867, THYMU3033402, THYMU3033626, THYMU3033630, THYMU3033649, THYMU3033754, THYMU3033759, THYMU3034078, THYMU3034099, THYMU3034453, THYMU3034853, THYMU3034867, THYMU3034983, THYMU3036200, THYMU3036310, THYMU3036934, THYMU3036953,

THYMU3037052, THYMU3037192, THYMU3037617, THYMU3037772, THYMU3037827, THYMU3037836, THYMU3037856, THYMU3037867, THYMU3037909, THYMU3037980, THYMU3038158, THYMU3038167, THYMU3038214, THYMU3038266, THYMU3038328, THYMU3038347, THYMU3038375, THYMU3038603, THYMU3038687, THYMU3038759, THYMU3038879, THYMU3038970, THYMU3039807, THYMU3039846, THYMU3040068, THYMU3040126, THYMU3040146, THYMU3040168, THYMU3040172, THYMU3040725, THYMU3040746, THYMU3040816, THYMU3040829, THYMU3040830, THYMU3040986, THYMU3041354, THYMU3041386, THYMU3041428, THYMU3041573, THYMU3041603, THYMU3041736, THYMU3041918, THYMU3042075, THYMU3042321, THYMU3042372, THYMU3042758, THYMU3042929, THYMU3043200, THYMU3043327, THYMU3043482,

THYMU3043597, THYMU3043688, THYMU3043779, THYMU3043883, THYMU3043993, THYMU3044175, THYMU3044188, THYMU3044441, THYMU3044445, THYMU3045510, THYMU3045673, THYMU3045692, THYMU3045704, THYMU3046140, THYMU3046350, THYMU3046360, THYMU3046856, THYMU3047115, THYMU3047144, THYMU3047156, THYMU3047366, THYMU3047513, THYMU3047542, THYMU3047760, THYMU3047891, TKIDN1000001, TKIDN1000044, TKIDN1000062, TKIDN1000066, TKIDN1000164, TKIDN1000171, TKIDN1000192, TKIDN2000127, TKIDN2000240, TKIDN2000246, TKIDN2000319, TKIDN2000464, TKIDN2000521, TKIDN2000701, TKIDN2001529, TKIDN2001662, TKIDN2002318, TKIDN2002329, TKIDN2002424, TKIDN2002632, TKIDN2002738, TKIDN2003044, TKIDN2003059, TKIDN2003062, TKIDN2003078,

TKIDN2003366, TKIDN2003396, TKIDN2003413, TKIDN2003597, TKIDN2003601, TKIDN2004185, TKIDN2004386, TKIDN2004458, TKIDN2004748, TKIDN2005487, TKIDN2005721, TKIDN2005934, TKIDN2005947, TKIDN2005956, TKIDN2006525, TKIDN2006761, TKIDN2006852, TKIDN2007605, TKIDN2007667, TKIDN2007828, TKIDN2008176, TKIDN2008778, TKIDN2009092, TKIDN2009330, TKIDN2009481, TKIDN2009641, TKIDN2009794, TKIDN2009889, TKIDN2010200, TKIDN2010232, TKIDN2010278, TKIDN2010602, TKIDN2010812, TKIDN2010934, TKIDN2011051, TKIDN2011160, TKIDN2011289, TKIDN2011324, TKIDN2012034, TKIDN2012771, TKIDN2012824, TKIDN2013134, TKIDN2013287, TKIDN2014570, TKIDN2014757, TKIDN2014771, TKIDN2014964, TKIDN2015025, TKIDN2015071, TKIDN2015161,

TKIDN2015263, TKIDN2015285, TKIDN2015423, TKIDN2015788, TKIDN2016309, TKIDN2016399, TKIDN2016846, TKIDN2018926, TKIDN2019116, TLIVE2000023, TLIVE2000142, TLIVE2000292, TLIVE2000762, TLIVE2000979, TLIVE2001327, TLIVE2001616, TLIVE2001684, TLIVE2001828, TLIVE2001927, TLIVE2002046, TLIVE2002336, TLIVE2002338, TLIVE2002562, TLIVE2002690, TLIVE2002882, TLIVE2003197, TLIVE2003200, TLIVE2003225, TLIVE2003381, TLIVE2003737, TLIVE2003961, TLIVE2003970, TLIVE2004110, TLIVE2004320, TLIVE2004331, TLIVE2004601, TLIVE2005180, TLIVE2005390, TLIVE2005516, TLIVE2005731, TLIVE2005866, TLIVE2006236, TLIVE2006359, TLIVE2006529, TLIVE2006621, TLIVE2006733, TLIVE2007000, TLIVE2007016, TLIVE2007132, TLIVE2007192,

TLIVE2007528, TLIVE2007607, TLIVE2007736, TLIVE2007816, TLIVE2007919, TLIVE2008083, TLIVE2008213, TLIVE2008221, TLIVE2008229, TLIVE2008712, TLIVE2008797, TLIVE2009087, TLIVE2009163, TLIVE2009489, TLIVE2009541, TLUNG2000179, TOVAR2000575, TOVAR2000649, TOVAR2000904, TOVAR2001281, TOVAR2001556, TOVAR2001730, TOVAR2002247, TOVAR2002514, TOVAR2002549, TOVAR2002800, TRACH1000011, TRACH1000018, TRACH1000057, TRACH1000074, TRACH1000100, TRACH1000106, TRACH1000125, TRACH1000140, TRACH1000144, TRACH1000193, TRACH1000212, TRACH1000240, TRACH2000002, TRACH2000015, TRACH2000024, TRACH2000032, TRACH2000045, TRACH2000047, TRACH2000081, TRACH2000083, TRACH2000148, TRACH2000185, TRACH2000196, TRACH2000235,

TRACH2000243, TRACH2000250, TRACH2000259, TRACH2000272, TRACH2000282, TRACH2000287, TRACH2000289, TRACH2000306, TRACH2000312, TRACH2000336, TRACH2000393, TRACH2000411, TRACH2000420, TRACH2000496, TRACH2000497, TRACH2000502, TRACH2000522, TRACH2000540, TRACH2000559, TRACH2000660, TRACH2000665, TRACH2000675, TRACH2000677, TRACH2000702, TRACH2000703, TRACH2000767, TRACH2000775, TRACH2000807, TRACH2000862, TRACH2000873, TRACH2000894, TRACH2000898, TRACH2000920, TRACH2000923, TRACH2000926, TRACH2000944, TRACH2000972, TRACH2000981, TRACH2000986, TRACH2001037, TRACH2001060, TRACH2001085, TRACH2001101, TRACH2001109, TRACH2001121, TRACH2001147, TRACH2001154, TRACH2001188, TRACH2001249, TRACH2001275,

TRACH2001314, TRACH2001335, TRACH2001339, TRACH2001341, TRACH2001347, TRACH2001395, TRACH2001400, TRACH2001403, TRACH2001416, TRACH2001428, TRACH2001474, TRACH2001478, TRACH2001523, TRACH2001525, TRACH2001532, TRACH2001592, TRACH2001607, TRACH2001621, TRACH2001646, TRACH2001651, TRACH2001684, TRACH2001898, TRACH2001933, TRACH2001968, TRACH2001996, TRACH2002043, TRACH2002092, TRACH2002100, TRACH2002138, TRACH2002333, TRACH2002664, TRACH2002784, TRACH2002803, TRACH2002840, TRACH2002847, TRACH2002853, TRACH2002862, TRACH2002954, TRACH2002960, TRACH2002988, TRACH2003070, TRACH2003347, TRACH2003484, TRACH2003516, TRACH2003605, TRACH2003736, TRACH2003759, TRACH2003941, TRACH2004039, TRACH2004054,

TRACH2004087, TRACH2004109, TRACH2004170, TRACH2004183, TRACH2004251, TRACH2004292, TRACH2004336, TRACH2004399, TRACH2004428, TRACH2004458, TRACH2004499, TRACH2004521, TRACH2004570, TRACH2004845, TRACH2004887, TRACH2004950, TRACH2005004, TRACH2005017, TRACH2005066, TRACH2005159, TRACH2005314, TRACH2005444, TRACH2005541, TRACH2005666, TRACH2005698, TRACH2005710, TRACH2005720, TRACH2005769, TRACH2005796, TRACH2005800, TRACH2006015, TRACH2006049, TRACH2006097, TRACH2006194, TRACH2006327, TRACH2006331, TRACH2006378, TRACH2006387, TRACH2006528, TRACH2006670, TRACH2006762, TRACH2006866, TRACH2006870, TRACH2006918, TRACH2007059, TRACH2007324, TRACH2007358, TRACH2007483, TRACH2007542, TRACH2007577,

TRACH2007674, TRACH2007676, TRACH2007688, TRACH2007733, TRACH2007754, TRACH2007816, TRACH2007894, TRACH2007969, TRACH2007988, TRACH2008081, TRACH2008112, TRACH2008113, TRACH2008127, TRACH2008130, TRACH2008278, TRACH2008472, TRACH2008478, TRACH2008540, TRACH2008583, TRACH2008597, TRACH2008716, TRACH2008820, TRACH2008891, TRACH2008921, TRACH2009006, TRACH2009060, TRACH2009107, TRACH2009123, TRACH2009268, TRACH2009291, TRACH2009310, TRACH2009340, TRACH2009348, TRACH2009358, TRACH2009661, TRACH2009730, TRACH2009743, TRACH2009754, TRACH2009851, TRACH2009862, TRACH2009934, TRACH2010159, TRACH2010218, TRACH2010251, TRACH2010272, TRACH2010317, TRACH2010419, TRACH2010451, TRACH2010505, TRACH2010587,

TRACH2010600, TRACH2010634, TRACH2010639, TRACH2010677, TRACH2010698, TRACH2010771, TRACH2010824, TRACH2010883, TRACH2010965, TRACH2010974, TRACH2011057, TRACH2011093, TRACH2011113, TRACH2011290, TRACH2011293, TRACH2011302, TRACH2011500, TRACH2011574, TRACH2011894, TRACH2011956, TRACH2012000, TRACH2012138, TRACH2012242, TRACH2012298, TRACH2012370, TRACH2012376, TRACH2012387, TRACH2012497, TRACH2012536, TRACH2012562, TRACH2012742, TRACH2012811, TRACH2012823, TRACH2012918, TRACH2013081, TRACH2013123, TRACH2013265, TRACH2013369, TRACH2013450, TRACH2013495, TRACH2013552, TRACH2013585, TRACH2013671, TRACH2013726, TRACH2013902, TRACH2013928, TRACH2013982, TRACH2013991, TRACH2014018, TRACH2014045,

TRACH2014046, TRACH2014077, TRACH2014082, TRACH2014268, TRACH2014284, TRACH2014337, TRACH2014356, TRACH2014371, TRACH2014483, TRACH2014495, TRACH2014544, TRACH2014573, TRACH2014725, TRACH2014760, TRACH2014815, TRACH2014839, TRACH2014938, TRACH2014950, TRACH2014972, TRACH2014974, TRACH2015180, TRACH2015243, TRACH2015316, TRACH2015381, TRACH2015486, TRACH2015654, TRACH2015824, TRACH2015939, TRACH2015987, TRACH2016080, TRACH2016131, TRACH2016286, TRACH2016317, TRACH2016347, TRACH2016380, TRACH2016384, TRACH2016410, TRACH2016498, TRACH2016533, TRACH2016551, TRACH2016554, TRACH2016651, TRACH2016690, TRACH2016709, TRACH2016722, TRACH2016835, TRACH2016980, TRACH2017086, TRACH2017368, TRACH2017498,

TRACH2017949, TRACH2017965, TRACH2017980, TRACH2018034, TRACH2018084, TRACH2018262, TRACH2018273, TRACH2018278, TRACH2018317, TRACH2018345, TRACH2018380, TRACH2018446, TRACH2018449, TRACH2018512, TRACH2018646, TRACH2018663, TRACH2018718, TRACH2018835, TRACH2018882, TRACH2018914, TRACH2019024, TRACH2019046, TRACH2019080, TRACH2019151, TRACH2019154, TRACH2019176, TRACH2019248, TRACH2019309, TRACH2019425, TRACH2019473, TRACH2019602, TRACH2019661, TRACH2019672, TRACH2019673, TRACH2019844, TRACH2019879, TRACH2020048, TRACH2020336, TRACH2020525, TRACH2021398, TRACH2021622, TRACH2021635, TRACH2021964, TRACH2022042, TRACH2022113, TRACH2022253, TRACH2022425, TRACH2022553, TRACH2022649, TRACH2022839,

TRACH2023246, TRACH2023299, TRACH2023306, TRACH2023416, TRACH2023479, TRACH2023684, TRACH2023860, TRACH2023876, TRACH2024139, TRACH2024187, TRACH2024271, TRACH2024293, TRACH2024408, TRACH2024512, TRACH2024559, TRACH2024647, TRACH2024651, TRACH2024730, TRACH2024735, TRACH2024749, TRACH2024821, TRACH2025057, TRACH2025084, TRACH2025155, TRACH2025224, TRACH2025344, TRACH2025379, TRACH2025507, TRACH2025535, TRACH2025569, TRACH2025705, TRACH2025749, TRACH2025753, TRACH2025810, TRACH2025853, TRACH2025911, TRACH2025932, TRACH3000014, TRACH3000017, TRACH3000043, TRACH3000134, TRACH3000180, TRACH3000342, TRACH3000420, TRACH3000515, TRACH3000530, TRACH3000548, TRACH3000558, TRACH3000586, TRACH3000780,

TRACH3000892, TRACH3000926, TRACH3001119, TRACH3001443, TRACH3001550, TRACH3001759, TRACH3002064, TRACH3002107, TRACH3002168, TRACH3002188, TRACH3002245, TRACH3002293, TRACH3002533, TRACH3002561, TRACH3002567, TRACH3002605, TRACH3002650, TRACH3002752, TRACH3002866, TRACH3002871, TRACH3002876, TRACH3002890, TRACH3003009, TRACH3003037, TRACH3003357, TRACH3003586, TRACH3003683, TRACH3003780, TRACH3003805, TRACH3003832, TRACH3004113, TRACH3004288, TRACH3004303, TRACH3004412, TRACH3004424, TRACH3004456, TRACH3004537, TRACH3004596, TRACH3004651, TRACH3004652, TRACH3004671, TRACH3004747, TRACH3004760, TRACH3004783, TRACH3004786, TRACH3004840, TRACH3004931, TRACH3004934, TRACH3005072, TRACH3005085,

TRACH3005102, TRACH3005173, TRACH3005191, TRACH3005211, TRACH3005274, TRACH3005294, TRACH3005479, TRACH3005549, TRACH3005702, TRACH3005808, TRACH3005852, TRACH3005896, TRACH3006038, TRACH3006149, TRACH3006228, TRACH3006264, TRACH3006336, TRACH3006379, TRACH3006392, TRACH3006395, TRACH3006397, TRACH3006412, TRACH3006470, TRACH3006589, TRACH3006626, TRACH3006699, TRACH3006717, TRACH3006889, TRACH3006985, TRACH3007097, TRACH3007268, TRACH3007274, TRACH3007277, TRACH3007391, TRACH3007541, TRACH3007595, TRACH3007625, TRACH3007627, TRACH3007689, TRACH3007766, TRACH3007866, TRACH3007995, TRACH3008042, TRACH3008061, TRACH3008093, TRACH3008201, TRACH3008508, TRACH3008535, TRACH3008629, TRACH3008688,

TRACH3008697, TRACH3008713, TRACH3008887, TRACH3008902, TRACH3008990, TRACH3009001, TRACH3009059, TRACH3009061, TRACH3009455, TRACH3009701, TRACH3009715, TRACH3009894, TRACH3009956, TRACH3010079, TRACH3010167, TRACH3010331, TRACH3010342, TRACH3010382, TRACH3010757, TRACH3010901, TRACH3011004, TRACH3011082, TRACH3011184, TRACH3011282, TRACH3011313, TRACH3011454, TRACH3011485, TRACH3011503, TRACH3011538, TRACH3011542, TRACH3012106, TRACH3012161, TRACH3012232, TRACH3012261, TRACH3012445, TRACH3012460, TRACH3012659, TRACH3012718, TRACH3012864, TRACH3012891, TRACH3012942, TRACH3013043, TRACH3013072, TRACH3013426, TRACH3013454, TRACH3013558, TRACH3013684, TRACH3013693, TRACH3013700, TRACH3013766,

TRACH3013900, TRACH3013926, TRACH3014063, TRACH3014183, TRACH3014215, TRACH3014316, TRACH3014415, TRACH3014580, TRACH3014641, TRACH3014885, TRACH3015136, TRACH3015336, TRACH3015346, TRACH3015354, TRACH3015467, TRACH3015626, TRACH3015951, TRACH3016051, TRACH3016120, TRACH3016264, TRACH3016277, TRACH3016368, TRACH3016455, TRACH3016614, TRACH3016805, TRACH3016992, TRACH3017171, TRACH3017409, TRACH3017934, TRACH3018108, TRACH3018191, TRACH3018240, TRACH3018261, TRACH3018519, TRACH3018524, TRACH3018606, TRACH3018783, TRACH3018907, TRACH3018933, TRACH3018943, TRACH3019018, TRACH3019058, TRACH3019142, TRACH3019290, TRACH3019347, TRACH3019370, TRACH3019447, TRACH3019598, TRACH3019621, TRACH3019662,

TRACH3019807, TRACH3019924, TRACH3019977, TRACH3020137, TRACH3020410, TRACH3020563, TRACH3020605, TRACH3020750, TRACH3020769, TRACH3020928, TRACH3020930, TRACH3021023, TRACH3021066, TRACH3021212, TRACH3021335, TRACH3021373, TRACH3021544, TRACH3021778, TRACH3021834, TRACH3021883, TRACH3022170, TRACH3022198, TRACH3022296, TRACH3022482, TRACH3022732, TRACH3022758, TRACH3022875, TRACH3022910, TRACH3022960, TRACH3023063, TRACH3023203, TRACH3023242, TRACH3023373, TRACH3023516, TRACH3023752, TRACH3023945, TRACH3023960, TRACH3024020, TRACH3024081, TRACH3024342, TRACH3024423, TRACH3024428, TRACH3024512, TRACH3024671, TRACH3024823, TRACH3025302, TRACH3025316, TRACH3025346, TRACH3025520, TRACH3026148,

TRACH3026283, TRACH3026299, TRACH3026303, TRACH3026542, TRACH3026650, TRACH3026676, TRACH3026744, TRACH3026949, TRACH3027229, TRACH3027527, TRACH3027681, TRACH3027701, TRACH3027885, TRACH3028031, TRACH3028164, TRACH3028180, TRACH3028195, TRACH3028441, TRACH3028597, TRACH3028678, TRACH3028837, TRACH3028847, TRACH3028855, TRACH3029139, TRACH3029329, TRACH3029451, TRACH3029462, TRACH3029520, TRACH3029592, TRACH3029629, TRACH3029665, TRACH3029670, TRACH3029800, TRACH3030111, TRACH3030130, TRACH3030176, TRACH3030252, TRACH3030725, TRACH3030855, TRACH3030883, TRACH3031316, TRACH3031400, TRACH3031463, TRACH3031479, TRACH3031518, TRACH3031660, TRACH3031678, TRACH3031936, TRACH3032044, TRACH3032065,

TRACH3032150, TRACH3032372, TRACH3032480, TRACH3032570, TRACH3032755, TRACH3032827, TRACH3032873, TRACH3033535, TRACH3033680, TRACH3033868, TRACH3034145, TRACH3034414, TRACH3034488, TRACH3034680, TRACH3034731, TRACH3034745, TRACH3034762, TRACH3034903, TRACH3035187, TRACH3035199, TRACH3035235, TRACH3035451, TRACH3035482, TRACH3035526, TRACH3036004, TRACH3036103, TRACH3036193, TRACH3036207, TRACH3036278, TRACH3036309, TRACH3036456, TRACH3036609, TRACH3036638, TRACH3036683, TRACH3036750, TRACH3036792, TRACH3036843, TRACH3036897, TRACH3036932, TRACH3036942, TRACH3036997, TRACH3037063, TRACH3037067, TRACH3037267, TRACH3037288, TRACH3037505, TRACH3037573, TRACH3037696, TRACH3037897, TRACH3038086,

TRACH3038399, TSTOM1000135, TSTOM1000186, TSTOM2000139, TSTOM2000235, TSTOM2000315, TSTOM2000442, TSTOM2000553, TSTOM2000569, TSTOM2000588, TSTOM2001195, TSTOM2001274, TSTOM2001571, TSTOM2001996, TSTOM2002265, TSTOM2002505, TSTOM2002561, TSTOM2002611, TSTOM2002672, TSTOM2002682

Homology Search Data

Homology search data for full-length nucleotide sequences and the deduced amino acid sequences are shown below. The search results contain two types of length unit for the sequences compared: aa and bb.

Each piece of data is punctuated with a double slash mark (//) and shown in order: Sequence Name, Definition of hit data, P value, Length of sequence compared, Homology, and Accession Number of hit data. Sequences which did not show hit data in the homology search list only the Clone name.

3NB692002685//Homo sapiens sperm acrosomal protein mRNA, complete cds.//
7.3E−117//227aa//100%//AF047437
3NB692002806//Dihydropteridine reductase (EC 1.6.99.7) (HDHPR) (Quinoid
dihydropteridine reductase).//1.00E−105//192aa//78%//P09417
3NB692008729
ACTVT2000380
ADIPS2000088//Human (hybridoma H210) anti-hepatitis A IgG variable region,
constant region, complementarity-determining regions mRNA, complete cds.//
3.5E−236//477aa//90%//M87789
ADRGL2000172
ADRGL2003329
ADRGL2009146
ADRGL2009691//Mus musculus D11Igp1 mRNA, complete cds.//5.1E−17//75aa//
60%//AF316996
ADRGL2009755//Homo sapiens brain and reproductive organ-expressed protein
(BRE) mRNA, complete cds.//2.2E−156//308aa//93%//AF015767
ADRGL2012038
ADRGL2012179
ASTRO1000009//BAND 4.1-LIKE PROTEIN 4 (NBL4 PROTEIN).//2.50E−45//309aa//
38%//O57457
ASTRO2002842//sentrin/SUMO-specific protease [Mus musculus]//2.00E−84//
148aa//54%//NM_030702
ASTRO2003960
ASTRO2014923//hypothetical protein//2.6E−66//133aa//100%//CAB70760
ASTRO2018373
ASTRO3000172
ASTRO3000177//Drosophila melanogaster BcDNA.GH03694 (BcDNA.GH03694) mRNA,
complete cds.//1.30E−89//385aa//47%//AF181626
ASTRO3000301//G2 [Homo sapiens]//0//1073aa//96%//AAA21253
ASTRO3000482
BLADE1000176
BLADE2001371
BLADE2001987
BLADE2002073
BLADE2002782//Rattus norvegicus clone C53 CDK5 activator-binding protein
mRNA, complete cds.//1.90E−24//70aa//82%//AF177476
BLADE2002947
BLADE2003474
BLADE2004089//Mus musculus PDZ domain actin binding protein Shroom mRNA,
complete cds.//4.20E−244//784aa//64%//AF199421
BLADE2004462
BLADE2004670
BLADE2005036//CARBOXYPEPTIDASE A1 PRECURSOR (EC 3.4.17.1).//1.20E−81//
168aa//93%//P15085
BLADE2005459
BLADE2007666
BLADE2007958//Homo sapiens U5 snRNP-specific 40 kDa protein mRNA, complete
cds//6.0E−91//190aa//99%//NP_004805
BLADE2008281
BLADE2008398//Homo sapiens LRR FLI-I interacting protein 2 (LRRFIP2) mRNA,
complete cds.//2.00E−98//200aa//99%//AF115509
BLADE2008539//tumor antigen SLP-8p [Homo sapiens]//0//427aa//95%//
NM_016516
BNGH42003570//Mus musculus mRNA for thrombospondin type 1 domain, complete
cds.//2.30E−44//239aa//39%//AB016768
BNGH42007798//VEGETATIBLE INCOMPATIBILITY PROTEIN HET-E-1.//4.1E−40//
242aa//37%//Q00808
BRACE1000186//g20 protein [Homo sapiens]//3.00E−74//138aa//85%//
NM_016210
BRACE1000258
BRACE1000533
BRACE1000572
BRACE2003639
BRACE2005457//PENDRIN.//1.30E−61//371aa//35%//O43511
BRACE2006319//Homo sapiens mRNA for Fln29, complete cds.//3E−157//283aa//
99%//AB007447
BRACE2008594//Human protein serine/threonine kinase stk2 mRNA, complete
cds.//1.90E−39//190aa//44%//L20321
BRACE2010489//Mus musculus nucleolar protein C7 mRNA, complete cds.//1.70E−149
//391aa//76%//AF324899
BRACE2011747
BRACE2014306//Chromaffin granule amine transporter (Vesicular amine
transporter 1) (VAT1).//1.00E−15//80aa//22%//P54219
BRACE2014475
BRACE2014657
BRACE2015058
BRACE2015314
BRACE2016981//Sarcolemmal-associated protein [Homo sapiens]//7.00E−18//
72aa//29%//NM_007159
BRACE2018762//Homer, neuronal immediate early gene, 3 [Homo sapiens]//
1.00E−144//267aa//77%//NM_004838
BRACE2024627
BRACE2026836//Mus musculus mRNA for smoothelin L1, large isoform.//8.00E−66
//220aa//55%//AJ010305
BRACE2027258//Homo sapiens E2a-Pbx1-associated protein (EB-1) mRNA, partial
cds.//5.80E−163//302aa//99%//AF145204
BRACE2027970
BRACE2028970
BRACE2029112//Homo sapiens mRNA for plexin-B1 plasma membrane receptor,
splice variant R (plexin-B1/SEP gene)//4.0E−99//119aa//62%//AJ011415
BRACE2029849
BRACE2030326//OOCYTE ZINC FINGER PROTEIN XLCOF6 (FRAGMENT).//1.40E−07//
106aa//33%//P18749
BRACE2030341//Homo sapiens protein kinase STK10 mRNA, partial cds.//2.60E−66
//129aa//100%//AF170723
BRACE2030884
BRACE2031154
BRACE2031389
BRACE2031527
BRACE2031531
BRACE2031899
BRACE2032044
BRACE2032329
BRACE2032385
BRACE2032538
BRACE2032823
BRACE2033720
BRACE2035381//Homo sapiens phospholipase A2, group IVB (cytosolic)
(PLA2G4B), mRNA//0//446aa//96%/NM_005090
BRACE2035441
BRACE2036005
BRACE2036096
BRACE2036830
BRACE2036834
BRACE2037847
BRACE2038114
BRACE2038329//Rattus norvegicus CBL-B (Cbl-b) mRNA, partial cds.//3.4E−96//
198aa//92%//AF199504
BRACE2038551//PROTEIN ARGININE N-METHYLTRANSFERASE 2 (EC 2.1.1.—).//6.40E−53
//102aa//100%//P55345
BRACE2039249//POLYPEPTIDE N-ACETYLGALACTOSAMINYLTRANSFERASE (EC 2.4.1.41)
(PROTEIN-UDP ACETYLGALACTOSAMINYLTRANSFERASE) (UDP-GALNAC: POLYPEPTIDE, N-
ACETYLGALACTOSAMINYLTRANSFERASE) (GALNAC-T1).//5.1E−99//199aa//88%//
Q10472
BRACE2039327//hypothetical protein FLJ20241//6.3E−47//97aa//100%//
XP_012789
BRACE2039475
BRACE2039734
BRACE2040138
BRACE2040325
BRACE2041009
BRACE2041200//PROTEIN-ARGININE DEIMINASE TYPE II (EC 3.5.3.15)
(PEPTIDYLARGININE DEIMINASE II) (KIAA0994).//5.9E−98//181aa//98%//Q9Y2J8
BRACE2041264
BRACE2042550//Bos taurus mRNA for SCO-spondin, partial.//1.8E−154//319aa//
80%//AJ133488
BRACE2043142
BRACE2043248//Human I(3)mbt protein homolog mRNA, complete cds.//7.1E−71//
140aa//98%//U89358
BRACE2043349
BRACE2043665
BRACE2044286//GUANINE NUCLEOTIDE EXCHANGE FACTOR DBS (DBL'S BIG SISTER)
(MCF2 TRANSFORMING SEQUENCE-LIKE PROTEIN).//7.20E−154//578aa//50%//Q64096
BRACE2044816
BRACE2044949
BRACE2045300//COFILIN, NON-MUSCLE ISOFORM.//3.9E−85//166aa//100%//P23528
BRACE2045428
BRACE2045596//CTD-BINDING SR-LIKE PROTEIN RA4 (FRAGMENT).//1.6E−08//
160aa//32%//Q63627
BRACE2045772//2-AMINO-3-KETOBUTYRATE COENZYME A LIGASE, MITOCHONDRIAL
PRECURSOR (EC 2.3.1.29) (AKB LIGASE) (GLYCINE ACETYLTRANSFERASE).//1.3E−107
//142aa//98%//O75600
BRACE2045947
BRACE2045954
BRACE2046251//Homo sapiens hucep-10 mRNA for cerebral protein-10, complete
cds.//4.3E−135//264aa//97%//AB000782
BRACE2046295//NTAK PROTEIN (NEURAL-AND THYMUS-DERIVED ACTIVATOR FOR ERBB
KINASES).//1.80E−165//304aa//99%//O14511
BRACE2047011
BRACE2047350
BRACE2047377//ras inhibitor [Homo sapiens]//4.40E−11//60aa//58%//
AAA36553
BRACE2047385
BRACE3000071
BRACE3000697
BRACE3000787
BRACE3000840//Baculoviral IAP repeat-containing protein 1 (Neuronal
apoptosis inhibitory protein).//0//979aa//96%//Q13075
BRACE3000973
BRACE3001002//Zinc finger X-linked protein ZXDB.//1.00E−45//97aa//70%//
P98169
BRACE3001217
BRACE3001391//Polycystin precursor (Autosomal dominant polycystic kidney
disease protein 1).//0//388aa//84%//P98161
BRACE3001595
BRACE3001754//Surfeit locus protein 5.//2.00E−87//166aa//83%//Q15528
BRACE3002298
BRACE3002390
BRACE3002508//WD40 repeat domain 11 protein [Homo sapiens]//1.00E−113//
200aa//98%//NM_018117
BRACE3003004
BRACE3003192//latent transforming growth factor beta binding protein 3 [Homo
sapiens]//0//670aa//91%//NM_021070
BRACE3003595//CRK-associated substrate (p130Cas) (Breast cancer anti-
estrogen resistance 1 protein).//0//712aa//84%//P56945
BRACE3003698
BRACE3004058//NADH-cytochrome B5 reductase (EC 1.6.2.2) (B5R).//1.00E−129//
220aa//96%//P00387
BRACE3004113
BRACE3004150//Heterogeneous nuclear ribonucleoprotein H (hnRNP H).//0//
390aa//86%//P31943
BRACE3004358
BRACE3004435
BRACE3004772//protein tyrosine phosphatase, receptor type, f polypeptide
(PTPRF), interacting protein (liprin), alpha 2 [Homo sapiens]//1.00E−101//
168aa//80%//NM_003625
BRACE3004783
BRACE3004843
BRACE3004880
BRACE3005145
BRACE3005225
BRACE3005430//DNA repair protein rad18.//4.00E−21//48aa//38%//P53692
BRACE3005499
BRACE3006185
BRACE3006226
BRACE3006462
BRACE3006872//Echinoderm microtubule-associated protein-like 2 (EMAP-2)
(HuEMAP-2).//1.00E−46//94aa//34%//O95834
BRACE3007322
BRACE3007472
BRACE3007480
BRACE3007559
BRACE3007625//espin [Rattus norvegicus]//1.00E−101//178aa//54%//
NM_019622
BRACE3007642
BRACE3007767
BRACE3008036
BRACE3008092
BRACE3008137//Interleukin-16 precursor (IL-16) (Lymphocyte chemoattractant
factor) (LCF).//0//530aa//83%//Q14005
BRACE3008384//rhomboid (veinlet, Drosophila)-like; Rhomboid, drosophila,
homolog of [Homo sapiens]//7.00E−94//158aa//51%//NM_003961
BRACE3008720//SPO0B-associated GTP-binding protein.//5.00E−33//80aa//
43%//P20964
BRACE3008772
BRACE3009090
BRACE3009237
BRACE3009297//mdgl-1 [Mus musculus]//6.00E−54//110aa//84%//AAG17143
BRACE3009377
BRACE3009574
BRACE3009701
BRACE3009708//Sodium/potassium-transporting ATPase alpha-2 chain precursor
(EC 3.6.3.9) (Sodium pump 2) (Na+/K+ ATPase 2).//0//868aa//98%//P50993
BRACE3009724
BRACE3009747//DAZ associated protein 1 [Homo sapiens]//2.00E−18//43aa//
57%//NM_018959
BRACE3010397//Glioma pathogenesis-related protein (RTVP-1 protein).//4.00E−29
//65aa//37%//P48060
BRACE3010428
BRACE3011271//Patched protein homolog 1 (PTC1) (PTC).//5.00E−84//147aa//
72%//Q13635
BRACE3011421//Diacylglycerol kinase, zeta (EC 2.7.1.107) (Diglyceride
kinase) (DGK-zeta) (DAG kinase zeta).//0//839aa//91%//Q13574
BRACE3011505
BRACE3012364
BRACE3012930
BRACE3013119
BRACE3013576
BRACE3013740
BRACE3013780
BRACE3014005//thymic stromal cotransporter: putative thymic stromal co-
transporter TSCOT [Mus musculus]//4.00E−21//71aa//23%//NM_021053
BRACE3014068
BRACE3014231
BRACE3014317
BRACE3014807//homolog 1; tweety homolog [Homo sapiens]//3.00E−38//75aa//
46%//NM_020659
BRACE3015027//Ras GTPase-activating protein nGAP.//3.00E−75//214aa//35%//
Q9UJF2
BRACE3015121
BRACE3015262//espin [Mus musculus]//1.00E−10//55aa//40%//AAF98134
BRACE3015521//EH-domain containing protein 1 (Testilin) (hPAST1).//1.00E−121
//215aa//93%//Q9H4M9
BRACE3015894
BRACE3016884//Glucoamylase S1/S2 precursor (EC 3.2.1.3) (Glucan 1,4-alpha-
glucosidase) (1,4-alpha-D-glucan glucohydrolase).//8.00E−07//87aa//17%//
P08640
BRACE3018308
BRACE3018963//Exostosin-like 3 (Putative tumour suppressor protein EXTL3)
(Multiple exostosis-like protein 3) (Hereditary multiple exostoses gene
isolog) (EXT-related protein 1).//6.00E−39//69aa//98%//O43909
BRACE3019055
BRACE3019084//protein tyrosine phosphatase, receptor type, f polypeptide
(PTPRF), interacting protein (liprin), alpha 1 [Homo sapiens]//1.00E−130//
226aa//93%//NM_003626
BRACE3020194
BRACE3020286
BRACE3020594//cytokine receptor-like factor 2; cytokine receptor CRL2
precusor [Homo sapiens]//1.00E−27//53aa//73%//NM_022148
BRACE3022769
BRACE3023912
BRACE3024073//hypothetical protein similar to small G proteins, especially
RAP-2A [Homo sapiens]//8.00E−36//77aa//48%//NM_021183
BRACE3024659
BRACE3024662
BRACE3025153
BRACE3025457//testis-specific protein TSP-NY [Homo sapiens]//0//413aa//
97%//NM_032573
BRACE3025531
BRACE3025630//Alkaline serine exoprotease A precursor (EC 3.4.21.—).//
4.00E−18//53aa//33%//P16588
BRACE3026008
BRACE3026075
BRACE3026735
BRACE3027242
BRACE3027326//RAP1 GTPase activating protein 1 (RAP1GAP).//1.00E−177//
331aa//50%//P47736
BRACE3027478
BRACE3030103
BRACE3031838
BRACE3032983
BRACE3040856//Golgi Transport; Got1p [Saccharomyces cerevisiae]//2.00E−13//
100aa//45%//NP_014020
BRACE3045033//polyamine-modulated factor 1; polyamine modulated factor 1
[Homo sapiens]//1.00E−41//82aa//100%//NM_007221
BRALZ2011796
BRALZ2012183
BRALZ2012848
BRALZ2014484
BRALZ2016085//MONOCARBOXYLATE TRANSPORTER 3 (MCT 3) (RETINAL EPITHELIAL
MEMBRANE PROTEIN).//4.2E−49//244aa//33%//Q90632
BRALZ2016498//Homo sapiens FKSG76 (FKSG76) mRNA, complete cds.//4.8E−65//
127aa//99%//AF345564
BRALZ2017359//Homo sapiens RNA helicase HDB/DICE1 mRNA, partial cds.//4.4E−62//
318aa//47%//AF141326
BRAMY2001473//Rattus norvegicus transmembrane receptor Unc5H2 mRNA, complete
cds.//3.00E−115//501aa//44%//U87306
BRAMY2003008
BRAMY2004771//CHONDROADHERIN PRECURSOR (CARTILAGE LEUCINE-RICH PROTEIN) (38 KDA
BONE PROTEIN).//2.30E−36//293aa//34%//Q27972
BRAMY2005052//COXSACKIEVIRUS AND ADENOVIRUS RECEPTOR PRECURSOR
(COXSACKIEVIRUS B-ADENOVIRUS RECEPTOR) (HCAR) (CVB3 BINDING PROTEIN).//
3.10E−37//200aa//35%//P78310
BRAMY2017528
BRAMY2019300//CHONDROADHERIN PRECURSOR (CARTILAGE LEUCINE-RICH PROTEIN) (38 KDA
BONE PROTEIN).//8.10E−32//350aa//28%//Q27972
BRAMY2019963
BRAMY2019985
BRAMY2020058
BRAMY2020270
BRAMY2021498//Mus musculus papilin mRNA, complete cds.//3E−89//546aa//
37%//AF314171
BRAMY2028856
BRAMY2028914
BRAMY2029602
BRAMY2030098
BRAMY2030109//Homo sapiens hucep-4 mRNA for cerebral protein-4, complete
cds.//7.6E−83//280aa//55%//D88259
BRAMY2030702
BRAMY2030703
BRAMY2030799
BRAMY2031317//Mus musculus semaphorin cytoplasmic domain-associated protein
3A (Semcap3) mRNA, complete cds.//2.6E−169//682aa//50%//AF127084
BRAMY2031377
BRAMY2031442
BRAMY2032014
BRAMY2032242
BRAMY2032317
BRAMY2033003//solute carrier family 22 (organic anion transporter), member
8//5.1E−25//60aa//100%//XP_006015
BRAMY2033116
BRAMY2033267//Rattus norvegicus mRNA for acetoacetyl-CoA synthetase,
complete//1.7E−42//148aa//66%//AB026291
BRAMY2033594
BRAMY2034185
BRAMY2034920
BRAMY2034993
BRAMY2036387
BRAMY2036396
BRAMY2036567//GUANINE NUCLEOTIDE EXCHANGE FACTOR DBS (DBL'S BIG SISTER)
(MCF2 TRANSFORMING SEQUENCE-LIKE PROTEIN).//5.3E−47//113aa//83%//Q64096
BRAMY2036699
BRAMY2036913
BRAMY2037823
BRAMY2038100
BRAMY2038484//Homo sapiens MDC-3. 13 isoform 2 mRNA, complete cds.//3.8E−65
//194aa//60%//AF099935
BRAMY2038846
BRAMY2038904//Homo sapiens mRNA for X-like 1 protein.//2.7E−199//738aa//
55%//AJ005821
BRAMY2039872//Homo sapiens protein phosphatase methylesterase-1 (PME-1) mRNA,
complete cds.//4.1E−43//84aa//98%//AF157028
BRAMY2040478
BRAMY2040592//Homo sapiens hucep-11 mRNA for cerebral protein-11, complete
cds.//2.20E−101//327aa//64%//AB001596
BRAMY2041261
BRAMY2041378
BRAMY2041542
BRAMY2042612
BRAMY2042641
BRAMY2042760
BRAMY2042918
BRAMY2044078
BRAMY2044246
BRAMY2045036
BRAMY2046478
BRAMY2046742
BRAMY2046989//Protein bimA.//4.00E−07//60aa//20%//P17885
BRAMY2047169
BRAMY2047420
BRAMY2047676
BRAMY2047746//nasopharyngeal carcinoma susceptibility protein [Homo
sapiens]//3.00E−18//50aa//42%//NM_013275
BRAMY2047751//REGULATOR OF G-PROTEIN SIGNALING 12 (RGs12).//1.3E−178//
336aa//100%//014924
BRAMY2047765
BRAMY2047884
BRAMY3000206
BRAMY3000213//Homo sapiens RaP2 interacting protein 8 (RPIP8) mRNA, complete
cds.//5.7E−85//167aa//99%//U93871
BRAMY3001401
BRAMY3001794//Rattus norvegicus Circadian Oscillatory Protein (SCOP)
(Scop)//1.00E−117//206aa//59%//NM_021657
BRAMY3002312
BRAMY3002620
BRAMY3002803//p21-activated protein kinase 6 [Homo sapiens]//0//598aa//
87%//NM_020168
BRAMY3002805
BRAMY3004224
BRAMY3004672
BRAMY3004900
BRAMY3004919//Adenylate cyclase, type V (EC 4.6.1.1) (ATP pyrophosphatelyase)
(Adenylyl cyclase).//0//831aa//91%//Q04400
BRAMY3005091//Phosphatidylinositol 4-kinase alpha (EC 2.7.1.67) (PI4-kinase)
(PtdIns-4-kinase) (PI4K-alpha).//1.00E−111//192aa//100%//P42356
BRAMY3005932//Diacylglycerol kinase, zeta (EC 2.7.1.107) (Diglyceride
kinase) (DGK-zeta) (DAG kinase zeta) (DGK-IV) (104 kDa diacylglycerol
kinase).//1.00E−35//71aa//98%//008560
BRAMY3006297
BRAMY3007206
BRAMY3007609//nuclear RNA export factor 2; TAP like protein 2 [Homo
sapiens]//2.00E−80//155aa//60%//NM_017809
BRAMY3008466//kinesin family member 21A [Mus musculus]//1.00E−167//277aa//
94%//NM_016705
BRAMY3008505
BRAMY3008650
BRAMY3009811
BRAMY3010411
BRAMY4000095//ERBB-3 receptor protein-tyrosine kinase precursor (EC
2.7.1.112) (Tyrosine kinase-type cell surface receptor HER3).//0//469aa//
80%//P21860
BRAMY4000229//CORONIN 2A (WD-REPEAT PROTEIN 2) (IR10).//2.40E−87//167aa//
71%//Q92828
BRAMY4000277//Alpha-1B-glycoprotein.//1.00E−163//276aa//100%//P04217
BRASW1000053
BRASW1000125
BRAWH1000127//Mus musculus mRNA for thrombospondin type 1 domain, complete
cds.//8.30E−49//204aa//43%//AB016768
BRAWH2001395//MYELIN BASIC PROTEIN (MBP).//7.90E−26//64aa//90%//P02686
BRAWH2001671
BRAWH2001940//H. sapiens gene from PAC 1026E2, partial.//0//640aa//98%//
AL035289
BRAWH2001973
BRAWH2002560//deleted in bladder cancer chromosome region candidate 1 [Homo
sapiens].//0//770aa//52%//NP_055433
BRAWH2002761
BRAWH2005315//neuronal-STOP protein//5.5E−119//441aa//56%//CAA75930
BRAWH2007658//COXSACKIEVIRUS AND ADENOVIRUS RECEPTOR HOMOLOG PRECURSOR
(MCAR).//8.80E−14//366aa//24%//P97792
BRAWH2010000//golgi autoantigen, golgin subfamily a, 3; golgin-160 [Homo
sapiens]//5.00E−15//148aa//23%//NM_005895
BRAWH2010084
BRAWH2010536//paraneoplastic antigen MA1//5.00E−18//100aa//42%//
NP_006020
BRAWH2012162//KE03 protein [Homo sapiens]//2.00E−97//330aa/55%//
AAC17109
BRAWH2012326
BRAWH2013294
BRAWH2013871
BRAWH2014414//Cadherin-4 precursor (Retinal-cadherin) (R-cadherin) (R-
CAD).//0//761aa//90%//P55283
BRAWH2014645
BRAWH2014662
BRAWH2014876
BRAWH2014954//Mus musculus neuronal IL-16 mRNA, complete cds.//4.40E−271//
604aa//84%//AF175292
BRAWH2016221//T-CELL RECEPTOR BETA CHAIN ANA 11.//2.90E−07//80aa//46%//
P06333
BRAWH2016439
BRAWH2016702
BRAWH2016724//MAP2 = HMW-MAP2 {alternatively spliced} [rats, brain, mRNA
Partial, 267 nt].//3.8E−24//74aa//77%//S81002
BRAWH3000078//Mus musculus adult male tongue cDNA, RIKEN full-length
enriched library, clone: 2310010M24, full insert sequence.//1.20E−29//
111aa//52%//AK009282
BRAWH3000100//dedicator of cyto-kinesis 1 [Homo sapiens]//1.00E−10//67aa//
20%//NM_001380
BRAWH3000314
BRAWH3000345
BRAWH3000491//40S ribosomal protein S12.//5.00E−42//90aa//84%//P46405
BRAWH3001326//5-azacytidine resistance protein azr1.//4.00E−27//74aa//
31%//Q09189
BRAWH3001475
BRAWH3001891
BRAWH3002574//Calpain 2, large [catalytic] subunit precursor (EC 3.4.22.17)
(Calcium-activated neutral proteinase) (CANP) (M-type) (M-calpain)
(Millimolar-calpain).//1.00E−84//156aa//91%//P17655
BRAWH3002600//cadherin 23; waltzer; otocadherin [Mus musculus]//3.00E−30//
139aa//28%//NM_023370
BRAWH3002819
BRAWH3002821//synaptotagmin-like 2 [Mus musculus]//0//358aa//78%//
NM_031394
BRAWH3003522
BRAWH3003555
BRAWH3003727
BRAWH3003801
BRAWH3003992
BRAWH3004453
BRAWH3004666
BRAWH3005132
BRAWH3005422
BRAWH3005912//MIC1 protein.//1.00E−29//71aa//28%//P53258
BRAWH3005981
BRAWH3006548
BRAWH3006792
BRAWH3007221
BRAWH3007506
BRAWH3007592
BRAWH3007726
BRAWH3007783
BRAWH3008341//Ubiquitin carboxyl-terminal hydrolase 6 (EC 3.1.2.15)
(Ubiquitin thiolesterase 6) (Ubiquitin-specific processing protease 6)
(Deubiquitinating enzyme 6) (Proto-oncogene TRE-2).//3.00E−63//123aa//
67%//P35125
BRAWH3008634
BRAWH3008697
BRAWH3008931
BRAWH3009297
BRCAN2002562//Splicing factor, arginine/serine-rich 2 (Splicing factor SC35)
(SC-35) (Splicing component, 35 kDa) (PR264 protein).//6.00E−47//88aa//
88%//Q62093
BRCAN2002856//AMYLOID BETA A4 PRECURSOR PROTEIN-BINDING FAMILY A MEMBER 2
(NEURON-SPECIFIC X11L PROTEIN) (NEURONAL MUNC18-1-INTERACTING PROTEIN 2)
(MINT-2) (ADAPTER PROTEIN X11BETA).//2.20E−89//191aa//93%//Q99767
BRCAN2002944//Mus musculus huntington yeast partner C (Hypc) mRNA, complete
cds.//2.1E−36//83aa//87%//AF135440
BRCAN2002948//Homo sapiens mRNA for gamma2-adaptin, complete cds.//6.6E−53
//117aa//97%//AB015318
BRCAN2003703//Homo sapiens mRNA for putative serine/threonine protein kinase,
partial.//1.4E−81//187aa//89%//AJ006701
BRCAN2003746//PHOSPHORYLASE B KINASE ALPHA REGULATORY CHAIN, SKELETAL MUSCLE
ISOFORM (PHOSPHORYLASE KINASE ALPHA M SUBUNIT).//5.9E−105//213aa//94%//
P46020
BRCAN2003987
BRCAN2004355
BRCAN2005436
BRCAN2006063//COCHL IN PRECURSOR (COCH-5B2).//5.7E−123//230aa//100%//
O43405
BRCAN2006290
BRCAN2006297
BRCAN2006450
BRCAN2007144
BRCAN2007409
BRCAN2007426
BRCAN2008528
BRCAN2009203//Homo sapiens liprin-alpha2 mRNA, complete cds.//1.2E−235//
273aa//93%//AF034799
BRCAN2009432//hypothetical protein similar to small G proteins, especially
RAP-2A [Homo sapiens]//1.00E−93//170aa//92%//NM_021183
BRCAN2010376
BRCAN2011254
BRCAN2011602
BRCAN2012355
BRCAN2012481
BRCAN2013655
BRCAN2013660//Arabidopsis thaliana putative protein (F4F15. 330) mRNA,
complete cds.//1.1E−24//138aa//44%//AF370547
BRCAN2014143
BRCAN2014602//DIACYLGLYCEROL KINASE, ZETA (EC 2.7.1.107) (DIGLYCERIDE
KINASE) (DGK-ZETA) (DAG KINASE ZETA).//6.5E−38//74aa//94%//Q13574
BRCAN2014881
BRCAN2015371
BRCAN2015464
BRCAN2016433
BRCAN2016619//CRK-associated substrate (p130Cas) (Breast cancer anti-
estrogen resistance 1 protein).//0//735aa//84%//P56945
BRCAN2017442//sarcosine dehydrogenase; dimethylglycine dehydrogenase-like 1
[Homo sapiens]//5.00E−20//51aa//59%//NM_007101
BRCAN2017717
BRCAN2017905
BRCAN2018935
BRCAN2019387
BRCAN2020710
BRCAN2021028
BRCAN2024451//REGULATOR OF G-PROTEIN SIGNALING 14 (RGS14) (FRAGMENT).//
1.5E−131//324aa//83%//O43566
BRCAN2024563
BRCAN2025712
BRCAN2028355//Ca2+/calmodulin-dependent protein kinase (EC 2.7.1.123) II
gamma-E//3E−254//479aa//98%//JC5636
BRCOC2000670
BRCOC2001505//MYELIN BASIC PROTEIN (MBP).//8.50E−26//64aa//90%//P02686
BRCOC2003213//Histidyl-tRNA synthetase (EC 6.1.1.21) (Histidine-tRNA
ligase) (HiaRS).//0//421aa//95%//P12081
BRCOC2007034
BRCOC2014033
BRCOC2016525
BRCOC2019934
BRCOC2020142
BRHIP2000691
BRHIP2000819//Human mRNA for actin binding protein p57, complete cds.//
1.3E−48//96aa//100%//D44497
BRHIP2000826
BRHIP2000920
BRHIP2001074
BRHIP2001805//Homo sapiens TREK-1 potassium channel (KCNK2) mRNA, complete
cds.//1.8E−49//134aa//80%//AF129399
BRHIP2001927//Mus musculus mRNA for HS1 binding protein 3.//2.7E−30//
102aa//68%//AJ132192
BRHIP2002122//Homo sapiens B aggressive lymphoma long isoform (BAL) mRNA,
complete cds.//1.8E−97//189aa//100%//AF307338
BRHIP2002172//Mus musculus urea transporter isoform UTA-3 mRNA, complete
cds.//6.9E−208//452aa//82%//AF258602
BRHIP2002346
BRHIP2003242
BRHIP2003786//CCA3 [Rattus norvegicus]//2.60E−199//603aa//61%//BAA19969
BRHIP2003917
BRHIP2004312
BRHIP2004359//ELAC PROTEIN.//6.80E−20//111aa//37%//Q47012
BRHIP2004814//Homo sapiens gibbon ape leukemia virus receptor 1 (SLC20A1)
gene, exon 11 and complete cds.//1.8E−188//346aa//99%//AF102063
BRHIP2004883
BRHIP2005236//latrophilin 2 splice variant baaae//1.3E−203//250aa//
97%//AAD05305
BRHIP2005354
BRHIP2005600
BRHIP2005719
BRHIP2005752//NG5 [Homo sapiens]//5.0E−61//200aa//100%//AAB47496
BRHIP2005932
BRHIP2006800
BRHIP2007616//plexin 2//7.5E−137//423aa//59%//BAA13189
BRHIP2007741
BRHIP2009340
BRHIP2009414//Bax inhibitor-1 (BI-1) (Testis enhanced gene transcript).//
3.00E−97//177aa//77%//P55061
BRHIP2009474
BRHIP2013699
BRHIP2014228
BRHIP2021615//Homo sapiens CUG-BP and ETR-3 like factor 4 (CELF4) mRNA,
complete cds.//9.60E−115//349aa//65%//AF329265
BRHIP2022221
BRHIP2024146
BRHIP2024165//Synthase [Homo sapiens]//5.00E−44//83aa//94%//NM_003896
BRHIP2026061
BRHIP2026288//Protein bem46.//2.00E−47//110aa//41%//P54069
BRHIP2029176
BRHIP2029393//COBW-like protein [Homo sapiens]//3.00E−89//158aa//98%//
NM_018491
BRHIP3000339//MYELIN BASIC PROTEIN (MBP).//8.5E−26//64aa//90%//P02686
BRHIP3000526
BRHIP3001283
BRHIP3006683
BRHIP3007483
BRHIP3007586
BRHIP3008183
BRHIP3008313//testis specific ankyrin-like protein 1 [Homo sapiens]//1.00E−120
//210aa//92%//NM_016552
BRHIP3008344
BRHIP3008405//Dynamin 2 (EC 3.6.1.50) (Dynamin UDNM).//1.00E−56//108aa//
90%//P39054
BRHIP3008565
BRHIP3008598
BRHIP3008997
BRHIP3009099
BRHIP3009448//2-19 protein precursor.//1.00E−102//179aa//99%//P98173
BRHIP3011241
BRHIP3013765
BRHIP3013897
BRHIP3015751
BRHIP3016213
BRHIP3018797
BRHIP3020182
BRHIP3024118//Monocarboxylate transporter 4 (MCT 4) (MCT 3).//1.00E−36//
108aa//30%//O35910
BRHIP3024533
BRHIP3024725
BRHIP3025161//Putative Rho/Rac guanine nucleotide exchange factor (Rho/Rac
GEF) (Faciogenital dysplasia protein homolog).//2.00E−75//175aa//30%//
P52734
BRHIP3025702
BRHIP3026097
BRHIP3027137//10-formyltetrahydrofolate dehydrogenase (EC 1.5.1.6) (10-
FTHFDH).//1.00E−119//208aa//93%//O75891
BRHIP3027854//Homo sapiens ectonucleotide pyrophosphatase/phosphodiesterase
2 (autotaxin) (ENPP2)//1.00E−130//222aa//94%//NM_006209
BRSSN2000684//CDC14 homolog B, isoform 3 [Homo sapiens]//3.00E−12//52aa//
30%//NM_033332
BRSSN2003086
BRSSN2004496//TASP for testis-specific adriamycin sensitivity protein [Homo
sapiens]//5.00E−45//101aa//39%//NM_018697
BRSSN2004719//SHC transforming protein.//4.00E−39//89aa//53%//P29353
BRSSN2006892
BRSSN2008549//oxysterol binding protein 2 [Mus musculus]//1.00E−149//
252aa//75%//NM_024289
BRSSN2008797
BRSSN2011262
BRSSN2011738
BRSSN2013874//TEMO [Rattus norvegicus]//2.00E−53//99aa//98%//NM_023986
BRSSN2014299//TPA inducible gene-1; TPA inducible protein [Homo sapiens]//
2.00E−47//93aa//86%//NM_015889
BRSSN2014424//transporter-like protein [Homo sapiens]//0//413aa//92%//
NM_022109
BRSSN2014556
BRSSN2018581
BRSSN2018925
BRSTN2000872//Protein disulfide isomerase A2 precursor (EC 5.3.4.1)
(PDIp).//0//341aa//92%//Q13087
BRSTN2001067
BRSTN2001613//HETEROGENEOUS NUCLEAR RIBONUCLEOPROTEINS C1/C2 (HNRNP C1 AND
HNRNP C2).//2.8E−34//214aa//43%//P07910
BRSTN2002400
BRSTN2003835
BRSTN2004863//Drosophila melanogaster polypeptide N-
acetylgalactosaminyltransferase mRNA, complete cds.//5.60E−126//526aa//
47%//AF158747
BRSTN2004987//Homo sapiens mRNA for mitochondrial tryptophanyl-tRNA
synthetase (WARS2 gene).//1.20E−162//360aa//86%//AJ242739
BRSTN2005721
BRSTN2006865
BRSTN2007000
BRSTN2007284
BRSTN2008052
BRSTN2008283
BRSTN2008418//Breakpoint cluster region protein (EC 2.7.1.—).//7.00E−33//
70aa//75%//P11274
BRSTN2008457
BRSTN2009899
BRSTN2010363
BRSTN2010500
BRSTN2010750
BRSTN2012320
BRSTN2012380
BRSTN2013741//Ras-related protein M-Ras (Ras-related protein R-Ras3).//
1.00E−105//189aa//90%//O14807
BRSTN2015015
BRSTN2016470
BRSTN2016678
BRSTN2017084
BRSTN2017110
BRSTN2017237
BRSTN2017771//Homo sapiens putative BTK-binding protein mRNA, complete
cds.//1.0E−41//90aa//99%//AF235049
BRSTN2018083
BRSTN2019129
BRTHA1000311
BRTHA2000855
BRTHA2001462
BRTHA2002115
BRTHA2002281//Rho guanine nucleotide exchange factor 10 [Homo sapiens].//
5.0E−26//123aa//39%//NP_055444.1
BRTHA2002376
BRTHA2002442
BRTHA2002493
BRTHA2002608//aldehyde dehydrogenase 1A3//2.00E−19//46aa//88%//NP_000684
BRTHA2002808//GAMMA-INTERFERON-INDUCIBLE PROTEIN IP-30 PRECURSOR.//7.8E−65
//141aa//90%//P13284
BRTHA2003030
BRTHA2003110//Protein Clorf8 precursor (Liver membrane-bound protein)
(HSPC001).//1.00E−98//17888//92%//Q9BXS4
BRTHA2003116
BRTHA2003461
BRTHA2004821
BRTHA2004978
BRTHA2005579//Xenopus laevis mRNA for Kielin, complete cds.//1.3E−190//
659aa//47%//AB026192
BRTHA2005956
BRTHA2006075
BRTHA2006146
BRTHA2006194
BRTHA2007122//ANKYRIN 2 (BRAIN ANKYRIN) (ANKYRIN B) (ANKYRIN,
NONERYTHROID).//6.1E−18//203aa//32%//Q01484
BRTHA2007422
BRTHA2007603//H. sapiens mRNA for BCL7B protein.//1.8E−56//116aa//98%//
X89985
BRTHA2008316
BRTHA2008335
BRTHA2008527//LUTROPIN-CHORIOGONADOTROPIC HORMONE RECEPTOR PRECURSOR (LH/CG-
R) (LSH-R) (LUTEINIZING HOROMINE RECEPTOR).//7.5E−66//189aa//73%//P22888
BRTHA2008535
BRTHA2008955
BRTHA2009311//EOSINOPHIL LYSOPHOSPHOLIPASE//1.0E−30//64aa//91%//P97400
BRTHA2009846
BRTHA2009972
BRTHA2010073
BRTHA2010608
BRTHA2010884
BRTHA2010907
BRTHA2011194
BRTHA2011351
BRTHA2011500
BRTHA2011641
BRTHA2012392//Homo sapiens HCDI (HCDI) mRNA, complete cds.//8.0E−95//
194aa//95%//AF226050
BRTHA2012562
BRTHA201298//INTERLEUKIN-13 RECEPTOR ALPHA-1 CHAIN PRECURSOR (IL-13R-ALPHA-
1) (IL-13RA-1).//1.5E−44//91aa//100%//P78552
BRTHA2013262
BRTHA2013460
BRTHA2013707
BRTHA2014792//ENHANCER OF ZESTE HOMOLOG 1 (ENX-2) (KIAA0388).//7.1E−21//
184aa//35%//Q92800
BRTHA2014828
BRTHA2015406//Homo sapiens mRNA for putative serine/threonine protein kinase,
partial.//1.90E−86//268aa//67%//AJ006701
BRTHA2015478
BRTHA2015696
BRTHA2015878
BRTHA2016215
BRTHA2016496//Vacuolar processing enzyme precursor (EC 3.4.22.—) (VPE).//
0//370aa//79%//P49043
BRTHA2016543
BRTHA2017353
BRTHA2017985
BRTHA2018165
BRTHA2018344
BRTHA2018591
BRTHA2018624//Oncorhynchus mykiss stl3 mRNA for rhamnose binding lectin STL3,
complete cds.//7.40E−21//167aa//34%//AB039024
BRTHA2018707
BRTHA2019014
BRTHA2019022
BRTHA2019048
BRTHA3000273
BRTHA3000297
BRTHA3000633//single-pass transmembrane protein [Rattus norvegicus]//5.00E−48
//220aa//54%//BAA90767
BRTHA3001721//TATA box binding protein (TBP)-associated factor, RNA
polymerase III, GTF3B subunit 2; TATA box binding protein (TBP)-associated
factor, RNA polymerase III, C, 90 kD; general transcription factor IIIB, 90 kD
[Homo sapiens]//4.00E−71//135aa//85%//NM_001519
BRTHA3002401
BRTHA3002427//Sodium- and chloride-dependent betaine transporter (Na+/Cl−
betaine/GABA transporter) (BGT-1).//0//553aa//96%//P48065
BRTHA3002933//uroplakin 3 [Homo sapiens]//1.00E−158//260aa//99%//
XP_001216
BRTHA3003074//putative prostate cancer susceptibility protein; hypothetical
protein FLJ10530 [Homo sapiens]//0//435aa//94%//NM_018127
BRTHA3003343//DAZ associated protein 1 [Homo sapiens]//1.00E−95//223aa//
92%//NP_061832
BRTHA3003449//MYOSIN HEAVY CHAIN, SMOOTH MUSCLE ISOFORM (SMMHC)
(FRAGMENT).//4.70E−215//400aa//100%//P35749
BRTHA3003474
BRTHA3003490
BRTHA3004475
BRTHA3005046
BRTHA3006856
BRTHA3007113
BRTHA3007148
BRTHA3007319
BRTHA3007769
BRTHA3008143
BRTHA3008310//Mus musculus mRNA for iroquois homeobox protein 6 (Irx6
gene).//1.20E−176//444aa//76%//AJ271055
BRTHA3008386
BRTHA3008520//sporulation-induced transcript 4-associated protein;
hypothetical protein FLJ11058 [Homo sapiens]//1.00E−164//287aa//87%//
NM_018312
BRTHA3008778//Acetyl-coenzyme A synthetase (EC 6.2.1.1) (Acetate-CoA
ligase) (Acyl-activating enzyme).//1.00E−168//286aa//51%//068040
BRTHA3009037//Regulator of G-protein signaling 3 (RGS3) (RGP3).//0//
479aa//92%//P49796
BRTHA3009090//neuropathy target esterase [Homo sapiens]//0//784aa//60%//
NM_006702
BRTHA3009291
BRTHA3010366
BRTHA3013884//Sorting nexin 14 (Fragment).//0//359aa//95%//Q9Y5W7
BRTHA3015815//Selenide,water dikinase 1 (EC 2.7.9.3) (Selenophosphate
synthetase 1) (Selenium donor protein 1).//1.00E−159//275aa//99%//P49903
BRTHA3015910
BRTHA3016845
BRTHA3016917//Valyl-tRNA synthetase 2 (EC 6.1.1.9) (Valine-tRNA ligase 2)
(VALRS 2).//4.00E−82//169aa//43%//P26640
BRTHA3017047
BRTHA3017589//junctional adhesion molecule 3 [Homo sapiens]//1.00E−119//
213aa//74%//NM_031470
BRTHA3017848//Organic cation/carnitine transporter 2 (Solute carrier family
22, member 5) (High-affinity sodium-dependent carnitine cotransporter).//
2.00E−42//105aa//35%//076082
BRTHA3018514
BRTHA3018617
BRTHA3018656
BRTHA3019105
CERVX1000042
CERVX2002006
COLON1000030
COLON2000470//Rattus norvegicus nucleolar protein C7C mRNA, complete cds.//
5.9E−51//187aa//49%//AF333986
COLON2000568//Ig alpha-2 chain C region.//0//324aa//95%//P01877
COLON2001721//GLUT4 vesicle protein [Mus musculus]//8.00E−36//160aa//
39%//AAD10190
COLON2002443
COLON2002520//Myosin heavy chain, nonmuscle type B (Cellular myosin heavy
chain, type B) (Nonmuscle myosin heavy chain-B) (NMMHC-B).//0//447aa//
70%//Q27991
COLON2003043
COLON2004478//protein Tro alpha1 H, myeloma//3.2E−233//475aa//88%//
0501254A
COLON2005126
COLON2005772//Homo sapiens candidate taste receptor T2R14 gene, complete
cds.//3.9E−54//112aa//97%//AF227138
COLON2006282
COLON2009499
CORDB1000140
CORDB2000061
CORDB2000541//F-actin capping protein beta subunit (CAPZ beta).//1.00E−126
//217aa//99%//P79136
CTONG1000087
CTONG1000088
CTONG1000288
CTONG1000302
CTONG1000341//THROMBOMODULIN PRECURSOR (FETOMODULIN) (TM) (CD141 ANTIGEN).//
1.0E−283//488aa//99%//P07204
CTONG1000467//Mus musculus mRNA for Deltex3, complete cds.//5.00E−54//
203aa//52%//AB015425
CTONG1000488
CTONG1000508
CTONG1000540
CTONG2000042//ALPHA-2-MACROGLOBULIN PRECURSOR (ALPHA-2-M).//2E−132//
841aa//35%//P01023
CTONG2001877
CTONG2004062//ATPase subunit 6 [Homo sapiens].//3.00E−71//226aa//91%//
BAA07295
CTONG2006798//putative serine/threonine protein kinase [Schizosaccharomyces
pombe]//5.80E−69//581aa//27%//CAB66438
CTONG2008233//Bos taurus DnaJ1 protein mRNA, complete cds.//0//1376 bp//
85%//AF308815
CTONG2009423//5-HYDROXYTRYPTAMINE 7 RECEPTOR (5-HT-7) (5-HT-X) (SEROTONIN
RECEPTOR) (5HT7).//2.40E−44//113aa//78%//P34969
CTONG2009531
CTONG2010803//Regulator of G-protein signaling 3 (RGS3) (RGP3).//0//
323aa//92%//P49796
CTONG2013178//Homo sapiens serine protease DESC1 (DESC1) mRNA, complete
cds.//2E−90//421aa//43%//AF064819
CTONG2017500//Homo sapiens muscle disease-related protein mRNA, complete
cds.//1.30E−59//239aa//47%//AF204674
CTONG2019248
CTONG2019652
CTONG2019704
CTONG2019788
CTONG2019833
CTONG2020026//Drosophila melanogaster BcDNA. GH09358 (BcDNA. GH09358) mRNA,
complete cds.//4.2E−187//669aa//45%//AF181639
CTONG2020127
CTONG2020522
CTONG2020638
CTONG2020806
CTONG2021132
CTONG2022153
CTONG2022601
CTONG2023021//H. sapiens mRNA for TFG protein.//2.3E−88//160aa//100%//
Y07968
CTONG2023512//Homo sapiens PIG-T mRNA for phosphatidyl inositol glycan class
T, complete cds.//7.1E−158//289aa//100%//AB057724
CTONG2024206
CTONG2024749//ALPHA-2-MACROGLOBULIN PRECURSOR (ALPHA-2-M).//1.1E−174//
699aa//46%//P06238
CTONG2025496//ALPHA-2-MACROGLOBULIN PRECURSOR (ALPHA-2-M).//1.2E−218//
977aa//45%//P01023
CTONG2025516//general transcription factor II, i, isoform 3; BTK-associated
protein, 135 kD; Williams-Beuren syndrome chromosome region 6; Bruton tyrosine
kinase-associated protein 135; TFII-I protein; SPIN protein [Homo sapiens]//
2.00E−28//57aa//89%//NM_033001
CTONG2025900
CTONG2026920
CTONG2027327
CTONG2028124//very long-chain acyl-CoA synthetase homolog 1; VLCS-H1 protein
[Homo sapiens]//5.00E−86//156aa//48%//NM_014031
CTONG2028687
CTONG3000084//PROBABLE GUANINE NUCLEOTIDE REGULATORY PROTEIN TIM (ONCOGENE
TIM) (P60 TIM) (TRANSFORMING IMMORTALIZED MAMMARY ONCOGENE).//4.3E−276//
519aa//100%//Q12774
CTONG3000657
CTONG3000686
CTONG3000707
CTONG3000896
CTONG3001123//Mus musculus Pax transcription activation domain interacting
protein PTIP mRNA, complete cds.//0//965aa//84%//AF104261
CTONG3001370//ALPHA-2-MACROGLOBULIN PRECURSOR (ALPHA2M).//1.5E−267//
1008aa//38%//Q61838
CTONG3001420
CTONG3001560
CTONG3002020
CTONG3002127//granuphilin [Mus musculus]//1.00E−104//204aa//49%//
NM_013757
CTONG3002412//Human DOCK180 protein mRNA, complete cds.//4.5E−236//678aa//
66%//D50857
CTONG3002674
CTONG3003179
CTONG3003483
CTONG3003652
CTONG3003654
CTONG3003737//PLATELET GLYCOPROTEIN V PRECURSOR (GPV) (CD42D).//1.80E−73//
434aa//37%//O08770
CTONG3003905
CTONG3003972
CTONG3004072//GL002 protein [Homo sapiens]//3.00E−80//152aa//88%//
NM_020193
CTONG3004712
CTONG3005325
CTONG3005648
CTONG3005713
CTONG3005813
CTONG3006067
CTONG3006186//syntaxin binding protein 4 [Mus musculus]//0//427aa//76%//
NM_011505
CTONG3006650
CTONG3007444
CTONG3007528
CTONG3007586
CTONG3007870
CTONG3008252
CTONG3008258//Homo sapiens GROS1-L protein mRNA, complete cds.//7.70E−177//
680aa//51%//AF097432
CTONG3008496
CTONG3008566
CTONG3008639//Human non-lens beta gamma-crystallin like protein (AIM1) mRNA,
partial cds.//0//836aa//99%//U83115
CTONG3008831//Rattus norvegicus PGC1 mRNA for PPAR gamma coactivator,
complete cds.//2.9E−69//176aa//46%//AB025784
CTONG3008894//Mus musculus SH3-domain binding protein 5//3.00E−42//89aa//
40%//NM_011894
CTONG3008951
CTONG3009028//sno gene product [Drosophila melanogaster]//1.00E−148//
1000aa//46%//AAF48240
CTONG3009227
CTONG3009239
CTONG3009328
CTONG3009385//Homo sapiens ARG99 mRNA, complete cds.//2.4E−77//153aa//
100%//AF319520
D3OST2002182//Homo sapiens mRNA for acetylglucosaminyltransferase-like
protein.//6.50E−11//265aa//23%//AJ007583
D3OST2002648//PUTATIVE G PROTEIN-COUPLED RECEPTOR GPR17 (R12).//3E−24//
184aa//28%//Q13304
D3OST3000169//Homo sapiens SH3-SAM adaptor protein (HACS1) mRNA, complete
cds.//2.40E−189//354aa//99%//AF218085
DFNES1000107
DFNES2000146//Mus musculus mRNA for thrombospondin type 1 domain, complete
cds.//4.10E−31//135aa//41%//AB016768
DFNES2001108//Homo sapiens nuclear dual-specificity phosphatase (SBF1) mRNA,
partial cds.//4.5E−41//134aa//63%//U93181
DFNES2005266//ADAM-TS 1 PRECURSOR (EC 3.4.24.—) (A DISINTEGRIN AND
METALLOPROTEINASE WITH THROMBOSPONDIN MOTIFS 1) (ADAMTS-1) (ADAM-TS1).//
4.80E−15//118aa//30%//P97857
DFNES2010502
DFNES2011239
DFNES2011499
ERLTF2000324
FCBBF1000297//Human protein immuno-reactive with anti-PTH polyclonal
antibodies mRNA, partial cds.//7.5E−186//359aa//99%//U28831
FCBBF2001183
FCBBF2007510
FCBBF3001977
FCBBF3002163//chromosome condensation-related SMC-associated protein 1;
chromosome condensation-related SMC-associated protein 1; KIAA0159 gene
product [Homo sapiens]//0//840aa//97%//NM_014865
FCBBF3003435
FCBBF3004502
FCBBF3004847
FCBBF3006171
FCBBF3007242
FCBBF3007540//GUANINE NUCLEOTIDE EXCHANGE FACTOR DBS (DBL'S BIG SISTER)//
5.00E−46//300aa//38%//O15068
FCBBF3008944
FCBBF3009888//Homo sapiens prostate stem cell antigen (PSCA) mRNA, complete
cds.//5.30E−06//122aa//32%//AF043498
FCBBF3012170//Mus musculus rostral cerebellar malformation protein (rcm)
mRNA, complete cds.//1.00E−80//325aa//51%//U72634
FCBBF3012288
FCBBF3013307//Homo sapiens RNA helicase-related protein mRNA, complete
cds.//0//644aa//99%//AF083255
FCBBF3013846
FCBBF3021576
FCBBF3021940//SYNAPSIN I (FRAGMENT).//5.00E−06//128aa//35%//O62732
FCBBF3023443
FCBBF3023895//contains simiarity to tubulin-tyrosine ligase [Caenorhabditis
elegans].//1.00E−54//220aa//39%//AAF39893
FCBBF3025730
FCBBF3027717
FCBBF4000076
FEBRA1000030//T-CELL RECEPTOR BETA CHAIN ANA 11.//2.7E−11//131aa//38%//
P06333
FEBRA2000253
FEBRA2006396
FEBRA2007544//transcription factor [Homo sapiens]//0//400aa//99%//
AAG33674
FEBRA2007708//DRA PROTEIN (DOWN-REGULATED IN ADENOMA).//2.60E−72//511aa//
34%//P40879
FEBRA2007793
FEBRA2007801//Homo sapiens TRIAD3 mRNA, partial cds.//2.3E−207//358aa//
99%//AF228527
FEBRA2008287
FEBRA2008311//GALANIN RECEPTOR TYPE 1 (GAL1-R) (GALR1).//1E−23//299aa//
27%//P56479
FEBRA2008360
FEBRA2008468//LYSOSOMAL ACID LIPASE/CHOLESTERYL ESTER HYDROLASE PRECURSOR
(EC 3.1.1.13) (LAL) (ACID CHOLESTERYL ESTER HYDROLASE) (STEROL ESTERASE)
(LIPASE A) (CHOLESTERYL ESTERASE).//1.20E−179//330aa//97%//P38571
FEBRA2010719
FEBRA2014213
FEBRA2015588
FEBRA2020484
FEBRA2020582
FEBRA2020668
FEBRA2020886
FEBRA2021339
FEBRA2021571
FEBRA2021908
FEBRA2021966
FEBRA2024136
FEBRA2024150
FEBRA2024343
FEBRA2024744//Homo sapiens Cat Eye Syndrome critical region protein isoform
1 mRNA, complete cds.//1.3E−126//252aa//94%//AF273270
FEBRA2025427
FEBRA2026984//TYROSYL-TRNA SYNTHETASE (EC 6.1.1.1) (TYROSYL-TRNA LIGASE)
(TYRRS) (FRAGMENT).//7.80E−271//528aa//94%//Q29465
FEBRA2027082
FEBRA2027297
FEBRA2027352
FEBRA2028366
FEBRA2028477
FEBRA2028618
HCASM2001301//MITOGEN-ACTIVATED PROTEIN KINASE 12 (EC 2.7.1.—)
(EXTRACELLULAR SIGNAL-REGULATED KINASE 6) (EC 2.7.1.—) (ERK6) (ERK5) (STRESS-
ACTIVATED PROTEIN KINASE-3) (MITOGEN-ACTIVATED PROTEIN KINASE P38 GAMMA) (MAP
KINASE P38 GAMMA).//2.2E−52//104aa//100%//P53778
HCASM2002502
HCASM2002918
HCASM2003212
HCASM2003415
HCASM2007047
HCASM2007737//SEC14-LIKE PROTEIN 1.//8.30E−09//162aa//24%//Q92503
HCHON2000028//Homo sapiens 7h3 protein mRNA, partial cds.//2.1E−94//
228aa//82%//AF209931
HCHON2000212
HCHON2000244
HCHON2000418
HCHON2000626//X-linked protein STS1769.//2.00E−47//89aa//83%//Q99871
HCHON2001084//ARABINOSE-PROTON SYMPORTER (ARABINOSE TRANSPORTER).//3E−66//
321aa//36%//P09830
HCHON2001217//Homo sapiens cullin CUL4B (CUL4B) mRNA, complete cds.//0//
782aa//99%//AF212995
HCHON2001548
HCHON2001577//Human elastin gene, exon 1.//1.5E−265//585aa//88%//M17282
HCHON2001712//Neutral amino acid transporter B(0) (ATB(0)).//0//369aa//
85%//Q15758
HCHON2002676//ALPHA-L-IDURONIDASE PRECURSOR (EC 3.2.1.76).//3.20E−274//
330aa//99%//P35475
HCHON2003532//PHOSPHORYLASE B KINASE BETA REGULATORY CHAIN (PHOSPHORYLASE
KINASE BETA SUBUNIT).//2.0E−159//312aa//95%//Q93100
HCHON2004007//Potential phospholipid-transporting ATPase IK (EC 3.6.3.13)
(Fragment).//1.00E−160//273aa//93%//O60423
HCHON2004531//UV excision repair protein RAD23 homolog B (HHR23B) (XP-C
repair complementing complex 58 kDa protein) (P58).//1.00E−142//270aa//
66%//P54727
HCHON2004776//transmembrane protein (63 kD), endoplasmic reticulum/Golgi
intermediate compartment [Homo sapiens]//0//500aa//86%//NP_006816
HCHON2005921//lipoma HMGIC fusion partner [Homo sapiens]//1.00E−15//52aa//
25%//NM_005780
HCHON2006250//Mus musculus SETA binding protein 1 (Sb1) mRNA, complete
cds.//3.9E−269//544aa//91%//AF246218
HCHON2006714
HCHON2007881
HCHON2008112//Homo sapiens HERC2 (HERC2) mRNA, complete cds.//1.80E−24//
79aa//70%//AF071172
HCHON2008444//28S ribosomal protein S15, mitochondrial precursor (MPR-S15)
(DC37).//5.00E−39//76aa//76%//P82914
HEART1000010//Hepatocyte growth factor-like protein precursor (Macrophage
stimulatory protein) (MSP) (Macrophage stimulating protein).//5.00E−18//
40aa//93%//P26927
HEART1000074//BANP homolog; putative transcription factor; Btg3 associated
nulcear protein [Mus musculus]//0//420aa//82%//NM_016812
HEART1000088
HEART1000139//TROPONIN T, CARDIAC MUSCLE ISOFORMS (TNTC).//1.40E−112//
221aa//98%//P45379
HEART2001680//Ig alpha-1 chain C region.//0//324aa//91%//P01876
HEART2001756
HEART2006131//2-hydroxyphytanoyl-CoA lyase [Mus musculus]//1.00E−138//
263aa//45%//NM_019975
HEART2006909//Hemolysin C.//3.00E−40//88aa//33%//Q54318
HEART2007031
HEART2010391
HEART2010492//GLYCEROL-3-PHOSPHATE ACYLTRANSFERASE, MITOCHONDRIAL PRECURSOR
(EC 2.3.1.15) (GPAT) (P90).//3.6E−47//462aa//32%//Q61586
HEART2010495//MICROTUBULE-ASSOCIATED PROTEIN 4.//2.00E−159//579aa//62%//
P27816
HHDPC1000118//Threonine synthase (EC 4.2.99.2).//3.00E−70//178aa//35%//
Q9ZMX5
HHDPC2001337
HLUNG1000017
HLUNG2000014//Mus musculus strain BALB/c dectin-2 alpha isoform mRNA,
complete cds.//2.80E−55//211aa//50%//AF240357
HLUNG2001996
HLUNG2002465//Homo sapiens Asef mRNA for APC-stimulated guanine nucleotide
exchange factor, complete cds.//1.30E−183//557aa//62%//AB042199
HLUNG2002958
HLUNG2003003
HLUNG2003872
HLUNG2010464
HLUNG2011041//basic proline-rich peptide IB-8a - human (fragments)//9.7E−07
//113aa//35%//D38355
HLUNG2011298//Homo sapiens cytochrome b5 reductase 1 (B5R.1) mRNA, complete
cds.//1.6E−27//79aa//78%//AF169481
HLUNG2012049
HLUNG2012287
HLUNG2012727
HLUNG2013204//phytoene dehydrogenase-like [Arabidopsis thaliana]//4.0E−53//
97aa//55%//BAB10768
HLUNG2013304
HLUNG2013622
HLUNG2013851
HLUNG2014262
HLUNG2014288//Mus musculus RP42 mRNA, complete cds.//2.4E−40//189aa//
43%//AF198092
HLUNG2014449
HLUNG2015617
HLUNG2017350//GAP JUNCTION ALPHA-3 PROTEIN (CONNEXIN 44) (CX44.//2.60E−53
//262aa//41%//P41987
HLUNG2017546
HLUNG2017806
HLUNG2019058
HSYRA2004858
HSYRA2005456
HSYRA2005496//ENDOGLIN PRECURSOR (CD105 ANTIGEN).//2.4E−117//245aa//92%//
P17813
HSYRA2006873
HSYRA2007667
HSYRA2008376
HSYRA2008714//POTENTIAL PHOSPHOLIPID-TRANSPORTING ATPASE ID (EC 3.6.1.—)
(FRAGMENT).//6.2E−158//412aa//70%//P98198
HSYRA2009075
HSYRA2009102//UDP-galactose transporter related [Homo sapiens].//3.0E−26//
280aa//32%//NP_005818
IMR322000127//ZINC FINGER PROTEIN 135.//3.30E−130//426aa//50%//P52742
IMR322000917//ZINC FINGER PROTEIN 29 (ZFP-29).//1.50E−34//197aa//40%//
Q07230
IMR322001380//Homo sapiens leucine-rich repeats containing F-box protein
FBL3 mRNA, complete cds.//7.00E−21//216aa//32%//AF186273
IMR322002035
IMR322002110
IMR322003675
IMR322006222
IMR322006495//Homo sapiens mRNA for kinetochore protein CENP-H, complete
cds.//3.1E−61//183aa//73%//AB035124
IMR322006886//Homo sapiens hepatocellular carcinoma-associated antigen 127
(HCA127) mRNA, complete cds.//2.5E−107//207aa//99%//AF270491
IMR322007225
IMR322016146
IMR322018117
KIDNE1000064//Mus musculus mRNA for RST, complete cds.//6.70E−219//552aa//
73%//AB005451
KIDNE2000665
KIDNE2000722
KIDNE2000832
KIDNE2000846//Mus musculus orphan transporter isoform A12 (Xtrp2) mRNA,
alternatively spliced, complete cds.//1.2E−54//203aa//50%//AF075262
KIDNE2001361//Mus musculus catp mRNA for cation-transporting atpase,
complete cds.//4.1E−123//273aa//91%//AB035381
KIDNE2001847//H. sapiens graf gene.//4.10E−98//300aa//55%//Y10388
KIDNE2002252//Drosophila melanogaster BcDNA. GH09358 (BcDNA. GH09358) mRNA,
complete cds.//6.30E−145//763aa//42%//AF181639
KIDNE2002991
KIDNE2003837
KIDNE2005543
KIDNE2006580//CYTOCHROME P450 4C1 (EC 1.14.14.1) (CYPIVC1).//1.10E−119//
496aa//49%//P29981
KIDNE2010264
KIDNE2011314
KIDNE2011532//similar to melanoma-associated chondroitin sulfate
proteoglycan 4//7.00E−30//54aa//60%//XP_000655
KIDNE2011635//Rabbit mRNA for sodium-glucose cotransporter, complete cds.//
2.1e−313//670aa//80%//D16226
KIDNE2012945//PROCOLLAGEN C-PROTEINASE ENHANCER PROTEIN PRECURSOR (PCPE)
(TYPE I PROCOLLAGEN COOH-TERMINAL PROTEINASE ENHANCER) (TYPE 1 PROCOLLAGEN C-
PROTEINASE ENHANCER PROTEIN).//2.00E−14//113aa//41%//Q15113
KIDNE2013095
LIVER2007415
LYMPB1000141
LYMPB2000083//HLA CLASS I HISTOCOMPATIBILITY ANTIGEN, ALPHA CHAIN F
PRECURSOR (HLAF ANTIGEN) (LEUKOCYTE ANTIGEN F).//4.80E−131//158aa//93%//
P33617
MESAN2001979
MESAN2006563
MESAN2012054
MESAN2014295
MESAN2015515
MESAN2018576
MESTC1000042
MESTC2000153
NB9N41000340
NCRRP1000129
NESOP2000744
NESOP2001433//ALC1_HUMAN Ig alpha-1 chain C region//0//353aa//100%//
P01876
NESOP2001656
NESOP2001694//H. sapiens graf gene.//7.4E−53//162aa//66%//Y10388
NESOP2001752
NESOP2002738
NHNPC2000606
NHNPC2000877
NHNPC2001223
NHNPC2001816
NHNPC2002565
NHNPC2002749
NOVAR2000136//Calsequestrin, skeletal muscle isoform precursor (Aspartactin)
(Laminin-binding protein).//1.00E−142//235aa//66%//P07221
NOVAR2000710
NOVAR2000962
NOVAR2001108//Human (hybridoma H210) anti-hepatitis A IgG variable region,
constant region, complementarity-determining regions mRNA, complete cds.//
3.0E−230//482aa//88%//M87789
NOVAR2001783
NT2NE2003252//Human putative serine/threonine protein kinase PRK (prk) mRNA,
complete cds.//3.00E−44//234aa//38%//U56998
NT2NE2005890
NT2NE2006531//ZINC FINGER PROTEIN 184 (FRAGMENT).//4.10E−113//437aa//
47%//Q99676
NT2NE2006909//Methionine aminopeptidase 2 (EC 3.4.11.18) (MetAP 2)
(Peptidase M2) (Initiation factor 2 associated 67 kDa glycoprotein) (P67).//
1.00E−147//258aa//80%//P50579
NT2NE2008060
NT2RI2003993
NT2RI2004618//Cytosolic acyl coenzyme A thioester hydrolase (EC 3.1.2.2)
(Long chain acyl-CoA thioester hydrolase) (CTE-II) (Brain acyl-CoA hydrolase)
(BACH).//1.00E−126//222aa//88%//O00154
NT2RI2005166//VEGETATIBLE INCOMPATIBILITY PROTEIN HET-E-1.//7.70E−14//
300aa//26%//Q00808
NT2RI2006686//E1A-ASSOCIATED PROTEIN P300.//1.30E−18//421aa//26%//Q09472
NT2RI2008724
NT2RI2009855
NT2RI2011422//Homo sapiens partial mRNA for transport-secretion protein 2.1
(TTS-2.1 gene).//6.4E−70//428aa//40%//AJ278475
NT2RI2011683
NT2RI2012659
NT2RI2012990//76.5 KDA PROTEIN C210RF13.//1.8E−73//149aa//100%//O95447
NT2RI2013357
NT2RI2014247
NT2RI2014551
NT2RI2014733
NT2RI2016128
NT2RI2018311
NT2RI2018883
NT2RI2019751
NT2RI2023303
NT2RI2025909//carnitine/acylcarnitine translocase//3.0E−32//260aa//37%//
NP_000378
NT2RI2025957//LU1 protein [Homo sapiens]//0//630aa//99%//AAF74512
NT2RI2027081
NT2RI2027396
NT2RI3000622
NT2RI3001263
NT2RI3001515//ALEX1 protein [Homo sapiens]//2.0E−25//220aa//29%//
NP_057692
NT2RI3002303
NT2RI3002842
NT2RI3002892
NT2RI3003031
NT2RI3003095
NT2RI3003162
NT2RI3003382
NT2RI3003409
NT2RI3004381
NT2RI3004510
NT2RI3005202
NT2RI3005403
NT2RI3005724
NT2RI3006132
NT2RI3006171//CARCINOEMBRYONIC ANTIGEN PRECURSOR (CEA) (MECONIUM ANTIGEN
100) (CD66E ANTIGEN).//1.3E−54//294aa//39%//P06731
NT2RI3006284//Homo sapiens chorea-acanthocytosis (CHAC) mRNA, complete
cds.//1.2E−144//538aa//51%//AF337532
NT2RI3006340//Myomesin 1 (Skelemin).//0//1390aa//81%//Q62234
NT2RI3006376
NT2RI3006673//LAR protein precursor (Leukocyte antigen related) (EC
3.1.3.48).//0//1151aa//90%//P10586
NT2RI3006796
NT2RI3007065
NT2RI3007158
NT2RI3007291
NT2RI3007543
NT2RI3007757//breast cancer nuclear receptor-binding auxiliary protein//
1.00E−172//295aa//94%//AAD21311
NT2RI3007978//CTP synthase II; CTP synthetase type 2 [Homo sapiens]//0//
536aa//91%//NM_019857
NT2RI3008055
NT2RI3008162
NT2RI3008652//Homo sapiens mRNA for CDEP, complete cds.//6.10E−113//
443aa//52%//AB008430
NT2RI3008697//erythroblast macrophage protein [Mus musculus]//2.00E−14//
70aa//25%//NM_021500
NT2RI3008974//probable transposase - human transposon MER37//1.20E−52//
165aa//69%//S72481
NT2RI3009158//Iroquois-class homeodomain protein IRX-3.//4.00E−16//52aa//
36%//P81067
NT2RP7000359//PROTEIN-TYROSINE PHOSPHATASE D1 (EC 3.1.3.48).//6.80E−25//
319aa//28%//Q16825
NT2RP7000466//Cegp1 protein//0//482aa//89%//NP_064436
NT2RP7004027//BONE MORPHOGENETIC PROTEIN 1 PRECURSOR (EC 3.4.24.—) (BMP-
1).//9.50E−33//301aa//30%//P98063
NT2RP7004123
NT2RP7005118//RAS GTPASE-ACTIVATING-LIKE PROTEIN IQGAP1 (P195) (KIAA0051).//
0//1034aa//58%//P46940
NT2RP7005529//PROBABLE GUANINE NUCLEOTIDE REGULATORY PROTEIN TIM (ONCOGENE
TIM) (P60 TIM) (TRANSFORMING IMMORTALIZED MAMMARY ONCOGENE).//5.40E−56//
364aa//37%//Q12774
NT2RP7005846
NT2RP7009030
NT2RP7009147//CHE-2 protein [Caenorhabditis elegans]//1.00E−177//740aa//
41%//CAB38019
NT2RP7009867
NT2RP7010128
NT2RP7010599//Homo sapiens endothelial lipase mRNA, complete cds.//5.60E−174
//321aa//98%//AF118767
NT2RP7011570
NT2RP7013795//VEGETATIBLE INCOMPATIBILITY PROTEIN HET-E-1.//4.90E−11//
129aa//34%//Q00808
NT2RP7014005//CTP synthase II; CTP synthetase type 2 [Homo sapiens]//0//
536aa//91%//NM_019857
NT2RP7015512
NT2RP7017365
NT2RP7017474
NT2RP7017546
NT2RP8000137
NT2RP8000296//similar to Kelch proteins//0//600aa//99%//AAF03529
NT2RP8000483//Rattus norvegicus mRNA for Nadrin E2, complete cds.//2.00E−208
//548aa//75%//AB060557
NTONG2000413//MATRIX METALLOPROTEINASE-16 PRECURSOR (EC 3.4.24.—) (MMP-16)
(MEMBRANE-TYPE MATRIX METALLOPROTEINASE 3) (MT-MMP 3) (MTMMP3) (MMP-X2).//
5.60E−62//290aa//37%//P51512
NTONG2003852
NTONG2005277//ANKYRIN 1 (ERYTHROCYTE ANKYRIN).//8.20E−31//363aa//31%//
Q02357
NTONG2005969
NTONG2006354
NTONG2007249
NTONG2007517//RING CANAL PROTEIN (KELCH PROTEIN).//9.10E−32//295aa//28%//
Q04652
NTONG2008088
NTONG2008672//final exon in repeat region; similar to long tandem repeat
region of sialidase (SP: TCNA_TRYCR, P23253) and neurofilament H protein//
1.9E−15//559aa//25%//AAC48204
OCBBF1000254
OCBBF2001794
OCBBF2002124//p40 [Homo sapiens]//3.00E−63//103aa//88%//AAC51270
OCBBF2003819
OCBBF2004826//T-cell lymphoma invasion and metastasis 2 [Homo sapiens]//0//
580aa//99%//NP_036586
OCBBF2004883
OCBBF2005428
OCBBF2006005//Bos taurus phosphatidic acid-preferring phospholipase A1 mRNA,
complete cds.//0//885aa//90%//AF045022
OCBBF2006058//Homo sapiens acyl-Coenzyme A dehydrogenase-8 precursor, mRNA,
complete cds.//5.40E−57//109aa//100%//AF126245
OCBBF2006151//Mus musculus protein tyrosine phosphatase-like protein PTPLB
(PtpIb) mRNA, complete cds.//3.40E−126//258aa//93%//AF169286
OCBBF2006567
OCBBF2006764//seizure related gene 6 [Mus musculus]//0//780aa//89%//
NP_067261
OCBBF2007028//Homo sapiens mRNA for NESCA, complete cds.//1.50E−169//
176aa//98%//AB026894
OCBBF2007068//ankyrin 1 [Bos taurus].//1.00E−68//800aa//32%//AAF61702
OCBBF2007114
OCBBF2007428
OCBBF2007478
OCBBF2007610//PSD-95/SAP90-associated protein-4 [Rattus norvegicus].//
1.00E−137//226aa//90%//AAB48590
OCBBF2008770
OCBBF2009788
OCBBF2009926
OCBBF2010140
OCBBF2010416
OCBBF2017516
OCBBF2019327
OCBBF2019823//lactate dehydrogenase A-like [Homo sapiens]//1.00E−164//
273aa//82%//NM_033195
OCBBF2020343
OCBBF2020453
OCBBF2020639
OCBBF2020741
OCBBF2020801//Ataxin 7 (Spinocerebellar ataxia type 7 protein).//5.00E−67//
116aa//100%//O15265
OCBBF2020838//FORKHEAD BOX PROTEIN D4 (FORKHEAD-RELATED PROTEIN FKHL9)
(FORKHEAD-RELATED TRANSCRIPTION FACTOR 5) (FREAC-5) (TRANSCRIPTION FACTOR
FKH-2).//1.70E−114//371aa//63%//Q60688
OCBBF2021020//Homo sapiens mRNA for vascular Rab-GAP/TBC-containing protein,
complete cds.//1.8E−24//107aa//47%//AB024057
OCBBF2021286
OCBBF2021323//Mus musculus GTRGEO22 (Gtrgeo22) mRNA, complete cds.//7.80E−49
//115aa//88%//AF303106
OCBBF2021788//Homo sapiens mRNA for B-cell CLL/lymphoma 9 (BCL9 gene).//
1.30E−92//600aa//42%//Y13620
OCBBF2022351//TIPD PROTEIN.//1.1E−54//263aa//40%//O15736
OCBBF2022574
OCBBF2023162
OCBBF2023643
OCBBF2024719
OCBBF2024781
OCBBF2024850
OCBBF2025028
OCBBF2025458
OCBBF2025527//GLYCEROL-3-PHOSPHATE DEHYDROGENASE [NAD+], CYTOPLASMIC (EC
1.1.1.8) (GPD-C) (GPDH-C).//8.60E−49//116aa//78%//P13707
OCBBF2025730
OCBBF2026645
OCBBF2027423
OCBBF2027478
OCBBF2028173//JM11 protein [Homo sapiens]//1.00E−131//304aa//97%//
AAF05832
OCBBF2028935
OCBBF2029901
OCBBF2030354//Mus musculus pantothenate kinase 1 beta (panK1beta) mRNA,
complete cds.//9.50E−195//372aa//96%//AF200357
OCBBF2030517
OCBBF2030574
OCBBF2030708
OCBBF2031167//Homo sapiens mRNA for MDC2 alpha, MDC2 beta, complete cds.//
0//813aa//99%//AB009671
OCBBF2031366
OCBBF2032590//H. sapiens mRNA for melanoma-associated chondroitin sulfate
proteoglycan (MCSP).//1.80E−11//151aa//39%//X96753
OCBBF2032599
OCBBF2032611
OCBBF2032671
OCBBF2033869//PROCOLLAGEN C-PROTEINASE ENHANCER PROTEIN PRECURSOR (PCPE)
(TYPE I PROCOLLAGEN COOH-TERMINAL PROTEINASE ENHANCER) (TYPE 1 PROCOLLAGEN C-
PROTEINASE ENHANCER PROTEIN).//6.6E−21//151aa//38%//Q15113
OCBBF2035110
OCBBF2035214
OCBBF2035564
OCBBF2035885
OCBBF2035916
OCBBF2036476
OCBBF2036743//ZINC FINGER PROTEIN 133.//9.00E−157//639aa//48%//P52736
OCBBF2037068//BCL2/adenovirus E1B 19-kDa protein-interacting protein 2.//
3.00E−74//122aa//66%//O54940
OCBBF2037340//Sacsin.//0//356aa//100%//Q9NZJ4
OCBBF2037398
OCBBF2037547//T-cell lymphoma invasion and metastasis 2 [Homo sapiens]//0//
1024aa//92%//NM_012454
OCBBF2037598//axonal-associated cell adhesion molecule [Mus musculus]//0//
366aa//89%//NP_031544
OCBBF2037638
OCBBF2038317//VPS10 domain receptor protein SORCS [Mus musculus]//0//
986aa//91%//NM_021377
OCBBF3000296
OCBBF3000483
OCBBF3002553
OCBBF3002600
OCBBF3003320//Potential phospholipid-transporting ATPase IS (EC 3.6.3.13)
(Fragment).//1.00E−110//179aa//62%//P98196
OCBBF3003592//Dynein beta chain, flagellar outer arm.//2.00E−54//222aa//
21%//Q39565
OCBBF3004314//Fas apoptotic inhibitory molecule [Mus musculus]//8.00E−67//
117aa//90%//NM_011810
OCBBF3006802
OCBBF3007516
OCBBF3008230
OCBBF3009279
PEBLM2000170//Sprouty homolog 3 (Spry-3).//1.00E−31//64aa//100%//O43610
PEBLM2000338
PEBLM2001465//diphthamide biosynthesis; Dph5p [Saccharomyces cerevisiae]//
9.00E−65//160aa//57%//NP_013273
PEBLM2001488
PEBLM2002594//ATP-binding cassette, sub-family A member 8 [Homo sapiens]//
4.50E−156//469aa//64%//XP_016390
PEBLM2002749
PEBLM2002887//ZINC FINGER PROTEIN 195.//1.50E−08//62aa//58%//O14628
PEBLM2004497
PEBLM2004666
PEBLM2005183//5′-3′ exonuclease//0//804aa//92%//CAA62819
PEBLM2005697
PEBLM2006113
PEBLM2007112
PEBLM2007140
PEBLM2007834
PERIC1000147
PERIC2000889//Rattus norvegicus dynamin-like protein variant 4 mRNA,
alternatively spliced, partial cds.//3.1E−22//51aa//98%//AF107048
PERIC2000914
PERIC2001227
PERIC2001228
PERIC2002766
PERIC2003090
PERIC2003452
PERIC2003699
PERIC2003720//kinectin 1; CG-1 antigen [Homo sapiens].//2.00E−92//270aa//
90%//NP_004977
PERIC2003834
PERIC2004028//Mus musculus erythroblast macrophage protein EMP mRNA,
complete cds.//3.80E−33//65aa//100%//AF263247
PERIC2004259
PERIC2004379
PERIC2004429
PERIC2004909
PERIC2005347//alpha 1C adrenergic receptor isoform 2//3.30E−22//74aa//
70%//BAA06901
PERIC2005370
PERIC2006035
PERIC2007914//Ubiquitously transcribed TPR gene on Y chromosome [Homo
sapiens]//1.0E−22//84aa//67%//NP_009056
PERIC2008385//sarcosine dehydrogenase; dimethylglycine dehydrogenase-like 1
[Homo sapiens]//4.00E−17//47aa//51%//NM_007101
PERIC2009086//Homo sapiens melanoma-associated antigen MG50 mRNA, partial
cds.//5.00E−189//508aa//66%//AF200348
PLACE5000001
PLACE5000171//E-SELECTIN PRECURSOR (ENDOTHELIAL LEUKOCYTE ADHESION MOLECULE
1) (ELAM-1) (LEUKOCYTE-ENDOTHELIAL CELL ADHESION MOLECULE 2) (LECAM2)
(CD62E).//1.50E−28//242aa//30%//P98110
PLACE5000260
PLACE5000282//elastin [Homo sapiens]//8.00E−08//420aa//97%//NP_000492
PLACE6001185
PLACE6009006
PLACE6012574
PLACE6019385//MITOGEN-ACTIVATED PROTEIN KINASE KINASE KINASE 5 (EC 2.7.1.—)
(MAPK/ERK KINASE KINASE 5) (MEK KINASE 5) (MEKK 5) (APOPTOSIS SIGNAL-
REGULATING KINASE 1) (ASK-1).//2E−57//92aa//63%//Q99683
PLACE6019932//Ictalurus punctatus NCC receptor protein 1 (NCCRP-1) mRNA,
complete cds.//1.2E−34//124aa//50%//AF208795
PLACE6020031//ANKYRIN HOMOLOG PRECURSOR.//2.70E−06//156aa//35%//Q06527
PLACE7000514//Mus musculus mRNA for ER protein 58 (EP58 gene).//3.80E−111//
366aa//55%//AJ404004
PLACE7001022
PLACE7001936
PLACE7002641//Ring assembly protein 3.//2.00E−13//79aa//26%//074994
PLACE7006051//cytoplasmic dynein heavy chain 2 [Rattus norvegicus]//0//
987aa//90%//NM_023024
PLACE7008431//Phosphatidylinositol-4-phosphate 5-kinase type II alpha (EC
2.7.1.68) (PIP5KII-alpha) (1-phosphatidylinositol-4-phosphate kinase)
(PtdIns(4)P-5-kinase B isoform) (Diphosphoinositide kinase).//1.00E−109//
200aa//56%//070172
PLACE7008623
PROST1000184//VASOACTIVE INTESTINAL POLYPEPTIDE RECEPTOR 1 PRECURSOR (VIP-R-
1) (PITUITARY ADENYLATE CYCLASE ACTIVATING POLYPEPTIDE TYPE II RECEPTOR)
(PACAP TYPE II RECEPTOR) (PACAP-R-2).//7.0E−63//125aa//98%//P32241
PROST1000528
PROST1000559//predicted osteoblast protein [Homo sapiens]//6.00E−33//
227aa//38%//NP_055703
PROST2003428//Protein pM5 precursor.//9.00E−47//91aa//89%//Q15155
PROST2008993//Mus musculus Pax transcription activation domain interacting
protein PTIP mRNA, complete cds.//1.10E−211//542aa//77%//AF104261
PROST2015243
PROST2016462//N-chimaerin (NC) (N-chimerin) (Alpha chimerin) (A-
chimaerin).//6.00E−26//65aa//34%//P30337
PROST2017367//PROTEIN-GLUTAMINE GLUTAMYLTRANSFERASE 4 (EC 2.3.2.13)
(PROSTATE TRANSGLUTAMINASE) (PROSTATE TRANSGLUTAMINASE) (TGP).//1.30E−52//
102aa//99%//P49221
PROST2017413
PROST2017700
PROST2018030
PROST2018090//SUSHI REPEAT-CONTAINING PROTEIN SRPX PRECURSOR.//9.50E−244//
414aa//99%//P78539
PROST2018511//Growth factor receptor-bound protein 7 (GRB7 adapter protein)
(Epidermal growth factor receptor GRB-7) (B47).//0//495aa//99%//Q14451
PROST2018902
PROST2018922
PROST2019296
PROST2019781
PUAEN2002489//Homo sapiens putative seven pass transmembrane protein
(TM7SF1) mRNA, complete cds.//1.0E−48//189aa//53%//AF027826
PUAEN2002616
PUAEN2003079//nasopharyngeal carcinoma susceptibility protein [Homo
sapiens]//3.00E−36//75aa//96%//NP_037407
PUAEN2005588
PUAEN2005930
PUAEN2006328//vascular Rab-GAP/TBC-containing [Homo sapiens]//8.0E−99//
360aa//53%//NP_008994
PUAEN2006701
PUAEN2007044//TRNA PSEUDOURIDINE SYNTHASE B (EC 4.2.1.70) (TRNA
PSEUDOURIDINE 55 SYNTHASE) (PSI55 SYNTHASE) (PSEUDOURIDYLATE SYNTHASE)
(URACIL HYDROLYASE).//7.90E−15//129aa//34%//P45142
PUAEN2007785
PUAEN2009174
PUAEN2009655//Bos taurus phosphatidic acid-preferring phospholipase A1 mRNA,
complete cds.//0//565aa//96%//AF045022
PUAEN2009795//Endothelial cell multimerin precursor.//1.00E−161//296aa//
78%//Q13201
PUAEN2009852//serine/threonine protein kinase Kp78 splice variant CTAK75a//
3.00E−33//86aa//36%//AAD48007
RECTM2000433//ZG-16p [Rattus norvegicus] //1.60E−64//148aa//85%//
CAA83059
RECTM2001347//sphingosine kinase type 2 isoform [Homo sapiens]//4.00E−46//
87aa//80%//NM_020126
SKMUS2000757
SKMUS2003074
SKMUS2004047
SKMUS2006394//Mus musculus ankyrin repeat-containing protein Asb-4 mRNA,
partial cds.//6.40E−54//405aa//34%//AF155355
SKNMC1000124//putative nuclear protein [Homo sapiens].//3.00E−12//398aa//
37%//NP_057689
SKNMC2002402
SKNMC2004457
SKNMC2004643
SKNMC2005772
SKNMC2006998//PROTEIN PHOSPHATASE INHIBITOR 1 (IPP-1) (I-1).//9.9E−32//
113aa//64%//Q13522
SKNMC2007504//DNA-directed RNA polymerase II largest subunit (EC 2.7.7.6)
(RPB1).//1.00E−16//76aa//26%//P08775
SKNMC2007961
SKNMC2009450
SKNSH2000482
SKNSH2009991
SKNSH2010015
SMINT1000192//PUTATIVE ATP-DEPENDENT RNA HELICASE KIAA0134.//4.00E−12//
37aa//100%//Q14147
SMINT2001818
SMINT2002743
SMINT2006641
SMINT2007391
SMINT2009902
SMINT2010076//Ig alpha-1 chain C region.//0//319aa//91%//P01876
SMINT2010897
SMINT2011311
SMINT2011888//protein Tro alpha1 H, myeloma//8.9E−215//481aa//82%//
0501254A
SMINT2015787//immunoglobulin lambda light chain [Homo sapiens]//1.40E−60//
164aa//77%//CAA40954
SPLEN2001599//Homo sapiens sialic acid binding immunoglobulin-like lectin 8
long splice variant (Siglec8) gene, complete cds.//4.00E−71//294aa//38%//
AF287892
SPLEN2002147//Halocynthia roretzi mRNA for HrPET-3, complete cds.//1.20E−09
//78aa//41%//AB029335
SPLEN2002467//Homo sapiens leucine-rich repeats containing F-box protein
FBL3 mRNA, complete cds.//1.60E−187//422aa//77%//AF186273
SPLEN2002707
SPLEN2006122//Homo sapiens RNA-binding region (RNP1, RRM) containing 2
(RNPC2)//2.00E−81//147aa//84%//NM_004902
SPLEN2009548
SPLEN2010912//putative nucleolar RNA helicase [Homo sapiens]//0//339aa//
90%//NM_019082
SPLEN2011422//CALDESMON (CDM).//5.3E−12//165aa//37%//Q05682
SPLEN2012624//BRCA1-associated RING domain protein 1 (BARD-1).//6.00E−14//
48aa//39%//Q9QZH2
SPLEN2012889//putative Na+-dependent inorganic phosphate cotransporter//
9.00E−19//70aa//32%//AAC35230
SPLEN2014946
SPLEN2015158
SPLEN2015267//Homo sapiens IGHG3 gene for immunoglobulin heavy chain gamma 3
constant region, 4-exon hinge, isolate Lib-A2.//1.0E−213//377aa//100%//
AJ390247
SPLEN2015679//Oryctolagus cuniculus sarcolemmal associated protein-3 mRNA,
complete cds.//4.90E−30//266aa//31%//U21157
SPLEN2016554
SPLEN2016863
SPLEN2017104
SPLEN2021701//HLA CLASS I HISTOCOMPATIBILITY ANTIGEN, A-2 ALPHA CHAIN
PRECURSOR.//4.40E−128//173aa//86%//P01892
SPLEN2023733
SPLEN2023791
SPLEN2024127
SPLEN2025491
SPLEN2027268
SPLEN2028844
SPLEN2028914
SPLEN2029051
SPLEN2029176
SPLEN2029522
SPLEN2029683
SPLEN2029727
SPLEN2029912
SPLEN2030335//Mus musculus fatty acid transport protein 3 mRNA, partial
cds//9.7E−251//275aa//81%//AF072758
SPLEN2030479
SPLEN2031125
SPLEN2031424
SPLEN2031547//Triose phosphate/phosphate translocator, non-green plastid
precursor (CTPT).//4.00E−20//76aa//25%//P52178
SPLEN2031724
SPLEN2031780
SPLEN2032154//NDRG1 PROTEIN (DIFFERENTIATION-RELATED GENE 1 PROTEIN) (DRG1)
(REDUCING AGENTS AND TUNICAMYCIN-RESPONSIVE PROTEIN) (RTP) (NICKEL-SPECIFIC
INDUCTION PROTEIN CAP43).//1.0E−22//80aa//57%//Q92597
SPLEN2032321
SPLEN2032813
SPLEN2033098//tumor necrosis factor receptor superfamily, member 14//1.7E−99
//183aa//100%//NP_003811
SPLEN2033153
SPLEN2033539
SPLEN2033921
SPLEN2034021
SPLEN2034081
SPLEN2034678
SPLEN2034781
SPLEN2036103
SPLEN2036326//CLAUDIN-5 (TRANSMEMBRANE PROTEIN DELETED IN VCFS) (TMDVCF).//
2.6E−118//218aa//100%//O00501
SPLEN2036712
SPLEN2036821//MITOCHONDRIAL CARNITINE/ACYLCARNITINE CARRIER PROTEIN
(CARNITINE/ACYLCARNITINE TRANSLOCASE) (CAC).//6.5E−10//104aa//33%//O43772
SPLEN2036932//Homo sapiens calcium and DAG-regulated guanine nucleotide
exchange factor I mRNA, complete cds.//3.9E−63//124aa//100%//AF081194
SPLEN2037194//NORQ PROTEIN.//5.5E−11//127aa//38%//Q51664
SPLEN2037580
SPLEN2037630
SPLEN2037722//lymphocyte antigen 108 [Mus musculus]//3.00E−63//137aa//
42%//NM_030710
SPLEN2038055
SPLEN2038180
SPLEN2038345
SPLEN2038407//basement membrane-induced gene//2.1E−33//283aa//34%//
XP_001646
SPLEN2039697
SPLEN2039936
SPLEN2040222
SPLEN2041304
SPLEN2041310
SPLEN2041645
SPLEN2041720
SPLEN2041977
SPLEN2042303
SPLEN2042598
STOMA1000189
STOMA2003444
STOMA2004294//Ig lambda chain V-IV region Bau.//1.00E−41//79aa//73%//
P01715
STOMA2004925
STOMA2008546//CDM PROTEIN (6C6-AG TUMOR-ASSOCIATED ANTIGEN) (DXS1357E).//
5.00E−124//246aa//100%//P51572
SYNOV1000374
SYNOV2005216//Homo sapiens laryngeal carcinoma related protein 1 mRNA,
complete cds.//2.5E−36//70aa//98%//AF268387
SYNOV2005448
SYNOV2005817//CYTOKINE RECEPTOR CLASS-II CRF2-4 PRECURSOR.//7.6E−176//
314aa//98%//Q08334
SYNOV2006430
SYNOV2007965//Homo sapiens mRNA for H-I(3)mbt-like protein, alternative
variant a.//3.1E−118//429aa//54%//AJ305226
SYNOV2012326//PUTATIVE PROTEIN-TYROSINE PHOSPHATASE TPTE (EC 3.1.3.48).//
6.7E−24//112aa//58%//P56180
SYNOV2014400//FIBULIN-1, ISOFORM C PRECURSOR.//4.0E−31//198aa//37%//
P23144
SYNOV2016124
SYNOV2017055
SYNOV2018921
SYNOV2021320//SH3 DOMAIN-BINDING PROTEIN 3BP-2.//2.3E−238//429aa//98%//
P78314
SYNOV3000231//Ig gamma-1 chain C region.//0//315aa//95%//P01857
SYNOV3000302//Ig gamma-1 chain C region.//1.00E−173//294aa//89%//P01857
SYNOV4000472
SYNOV4000706//B cell phosphoinositide 3-kinase adaptor [Mus musculus]//0//
633aa//79%//NM_031376
SYNOV4001326
SYNOV4001395
SYNOV4002346
SYNOV4002392
SYNOV4002883//S-adenosylmethionine decarboxylase proenzyme (EC 4.1.1.50)
(AdoMetDC) (SamDC) [Contains: S-adenosylmethionine decarboxylase alpha chain;
S-adenosylmethionine decarboxylase beta chain].//4.00E−72//129aa//99%//
P17707
SYNOV4003322
SYNOV4004184
SYNOV4004741//BENE protein (Fragment).//2.00E−77//140aa//94%//Q13021
SYNOV4004823
SYNOV4004914
SYNOV4006256
SYNOV4007012
SYNOV4007215
SYNOV4007360//SSXT protein (SYT protein).//5.00E−24//70aa//36%//Q62280
SYNOV4007430
SYNOV4007521//fibroblast growth factor receptor-like 1 precursor [Homo
sapiens]//7.00E−11//53aa//29%//NM_021923
SYNOV4007553//toll-like receptor2 [Homo sapiens]//0//740aa//94%//
NM_003264
SYNOV4007671//Syntaxin 3.//1.00E−144//262aa//99%//Q13277
SYNOV4008336
SYNOV4008440//Protein BAP28.//0//1119aa//85%//Q9H583
T1ESE2000116
TBAES2001171
TBAES2001220
TBAES2001229//60S ribosomal protein L23a.//8.00E−48//92aa//82%//P29316
TBAES2001258//SERINE PROTEASE HEPSIN (EC 3.4.21.—) (TRANSMEMBRANE PROTEASE,
SERINE 1).//6.40E−19//55aa//87%//P05981
TBAES2001492
TBAES2001751
TBAES2002197
TBAES2003550
TBAES2004055//NY-REN-50 antigen//1.00E−155//290aa//99%//AAD42878.
TBAES2005157
TBAES2005543
TBAES2006568
TBAES2007964
TCERX2000613
TCOLN2002278
TESOP1000127
TESOP2000801//PROTO-ONCOGENE TYROSINE-PROTEIN KINASE YES (EC 2.7.1.112)
(P61-YES) (C-YES).//3.9E−46//159aa//57%//Q04736
TESOP2001122//Caenorhabditis elegans LIN-9S (lin-9) mRNA, complete cds.//
5.60E−25//222aa//28%//AF269694
TESOP2001166//Mus musculus SOCS-5 mRNA, complete cds.//1.2E−114//439aa//
53%//AF033187
TESOP2001345
TESOP2001605//Homo sapiens laryngeal carcinoma related protein 1 mRNA,
complete cds.//2.5E−36//70aa//98%//AF268387
TESOP2001818
TESOP2001849
TESOP2001865
TESOP2001953//ooplasm [Mus musculus]//7.00E−08//58aa//26%//NM_011860
TESOP2002273
TESOP2002451
TESOP2002489
TESOP2002539
TESOP2002950
TESOP2003273
TESOP2003753
TESOP2004114//PROCOLLAGEN-LYSINE, 2-OXOGLUTARATE 5-DIOXYGENASE 2 PRECURSOR
(EC 1.14.11.4) (LYSYL HYDROXYLASE 2) (LH2).//1.70E−202//237aa//99%//
O00469
TESOP2005285//Homo sapiens partial mRNA for chr2 synaptotagmin (CHR2SYT
gene).//1.1E−21//54aa//96%//AJ303365
TESOP2005485//Ig delta chain C region.//2.00E−77//136aa//100%//P01880
TESOP2005579
TESOP2006041
TESOP2006060
TESOP2006068
TESOP2006670
TESOP2006746
TESOP2007052
TESOP2007262
TESOP2007636
TESOP2007688
TESOP2009121//Homo sapiens centromere protein E (312 kD) (CENPE), mRNA//
2.00E−10//155aa//20%//NM_001813
TESOP2009555
TESTI1000257//GLUCOSE TRANSPORTER TYPE 3, BRAIN.//7.4E−249//493aa//95%//
P11169
TESTI1000319//Putative eukaryotic translation initiation factor 3 subunit
(elF-3) (Fragment).//0//683aa//97%//O75153
TESTI1000330
TESTI1000348
TESTI1000390
TESTI1000491
TESTI1000545//Ring assembly protein 3.//2.00E−14//92aa//26%//O74994
TESTI2000443
TESTI2000644//SMALL INDUCIBLE CYTOKINE A14 PRECURSOR (CHEMOKINE CC-1/CC-3)
(HCC-1/HCC-3) (NCC-2).//2.80E−36//69aa//98%//Q16627
TESTI2002036//DIHYDROPYRIDINE-SENSITIVE L-TYPE, SKELETAL MUSCLE CALCIUM
CHANNEL ALPHA-1 SUBUNIT.//1.70E−18//398aa//24%//P22316
TESTI2002618//ADAM 2 PRECURSOR (A DISINTEGRIN AND METALLOPROTEINASE DOMAIN
2) (FERTILIN BETA SUBUNIT) (PH-30) (PH30).//1.10E−57//253aa//47%//Q99965
TESTI2002928
TESTI2003347//Homo sapiens connexin 59 (CX59) gene, complete cds.//1.80E−243
//440aa//100%//AF179597
TESTI2003573//Mus musculus cell cycle checkpoint control protein Mrad9 gene,
complete cds.//2.4E−38//325aa//30%//AF045662
TESTI2004215//Maackia amurensis early nodulin (ENOD2) mRNA, partial cds.//
1.3E−34//390aa//31%//AF039708
TESTI2004700
TESTI2005376
TESTI2005610//H. sapiens encoding CLA-1 mRNA.//5.9E−234//425aa//99%//
Z22555
TESTI2005739//Drosophila melanogaster Rho-kinase (Rhk) mRNA, complete cds.//
1.7E−09//383aa//24%//AF151375
TESTI2005986
TESTI2006041
TESTI2006643
TESTI2006648//ATP-binding cassette, sub-family C, member 5a//9E−109//
452aa//39%//NP_038818
TESTI2009474
TESTI2009477//TRICHOHYALIN.//1.9E−18//124aa//39%//P37709
TESTI2009511
TESTI2009812
TESTI2010400
TESTI2013381
TESTI2013382
TESTI2014716//G-RICH SEQUENCE FACTOR-1 (GRSF-1).//2.6E−228//391aa//99%//
Q12849
TESTI2014843
TESTI2016046//Homo sapiens HOTTL protein mRNA, complete cds.//2.8E−20//
242aa//26%//AF078842
TESTI2017727
TESTI2018838
TESTI2019042
TESTI2019648
TESTI2023254
TESTI2023599
TESTI2024567//METABOTROPIC GLUTAMATE RECEPTOR 8 PRECURSOR.//1.10E−130//
243aa//99%//000222
TESTI2026505//PUTATIVE RHO/RAC GUANINE NUCLEOTIDE EXCHANGE FACTOR (RHO/RAC
GEF) (FACIOGENITAL DYSPLASIA PROTEIN).//1.40E−50//378aa//29%//P98174
TESTI2027019//Homo sapiens leucine-rich repeat-containing G protein-coupled
receptor 6 (LGR6) mRNA, partial cds.//4.80E−125//137aa//100%//AF190501
TESTI2031529
TESTI2034520//Rattus norvegicus SMC (segregation of mitotic chromosomes 1)-
like 1 (yeast) (Smc1l1), mRNA//1.00E−145//250aa//53%//NM_031683
TESTI2034749
TESTI2034767//Homo sapiens collagen type IX alpha 1 chain (COL9A1) gene,
long and short alternatively spliced forms, exon 38 and complete cds.//
1.40E−191//484aa//73%//AF036130
TESTI2034953//Homo sapiens 88-kDa Golgi protein (GM88) mRNA, complete cds.//
2.00E−27//91aa//64%//AF204231
TESTI2034997
TESTI2035107
TESTI2035997
TESTI2036513
TESTI2036684
TESTI2037643
TESTI2040018//Homo sapiens ZNF258 (ZNF258) mRNA, complete cds.//7.80E−97//
461aa//49%//AF055470
TESTI2042450
TESTI2044796//ring finger protein 3 [Homo sapiens]//9.00E−41//92aa//38%//
NM_006315
TESTI2044833
TESTI2045920
TESTI2045983
TESTI2046347
TESTI2047071
TESTI2048465
TESTI2048603
TESTI2048898
TESTI2049206
TESTI2049246
TESTI2049277
TESTI2049422
TESTI2049452
TESTI2049469
TESTI2049576
TESTI2049857//golgi stacking protein homolog GRASP55 [Rattus norvegicus]//
5.00E−163//410aa//89%//AAD55350
TESTI2050137//SHC transforming protein.//1.00E−113//232aa//54%//P98083
TESTI2050681
TESTI2050987//RET finger protein-like 1.//4.00E−35//94aa//34%//075677
TESTI2051279
TESTI2051488
TESTI2051543
TESTI2051767
TESTI2051806
TESTI2051867//60S ribosomal protein L4 (L1).//1.00E−126//222aa//86%//
P36578
TESTI2052211
TESTI2052693//brk kinase substrate [Homo sapiens].//0//341aa//87%//
CAB65105
TESTI2052698
TESTI2052822
TESTI2053242
TESTI2053399//Homo sapiens pescadillo homolog 1, containing BRCT domain
(zebrafish) (PES1), mRNA//9.00E−33//63aa//100%//NM_014303
TESTI2053526
TESTI2053621//Guanylyl cyclase activating protein 1 (GCAP 1) (Guanylate
cyclase activator 1A).//7.00E−96//170aa//92%//P43080
TESTI4000014//130 kDa leucine-rich protein (LRP 130) (GP130).//0//1210aa//
96%//P42704
TESTI4000068
TESTI4000079//nuclear dual-specificity phosphatase [Homo sapiens]//6.00E−07
//80aa//36%//AAC39675
TESTI4000209//Homo sapiens F-BOX domain protein mRNA, complete cds.//5.5E−103
//194aa//99%//AF248640
TESTI4000215
TESTI4000250
TESTI4000288//Dynamin-1 (EC 3.6.1.50) (D100) (Dynamin, brain) (B-dynamin).//
2.00E−13//38aa//77%//P21575
TESTI4000349//thyroid hormone receptor interactor 12//1.00E−39//180aa//
40%//NP_004229
TESTI4000462
TESTI4000530
TESTI4000724//solute carrier family 16 (monocarboxylic acid transporters)//
5.00E−47//490aa//28%//NP_004687
TESTI4000970
TESTI4001100//protein tyrosine phosphatase, receptor type, f polypeptide
(PTPRF), interacting protein (liprin), alpha 1 [Homo sapiens]//3.00E−21//
50aa//40%//NM_003626
TESTI4001106//ubiquitin-protein ligase e3 componen n-recognin [Mus
musculus]//1.00E−124//228aa//45%//NM_009461
TESTI4001148//Dynein beta chain, ciliary.//1.00E−152//282aa//45%//P39057
TESTI4001176//Regulator of nonsense transcripts 1 (Nonsense mRNA reducing
factor 1) (NORF1) (Up-frameshift suppressor 1 homolog).//3.00E−46//90aa//
92%//Q92900
TESTI4001201
TESTI4001206
TESTI4001527//UDP-glucuronosyltransferase 2C1 microsomal (EC 2.4.1.17)
(UDPGT) (Fragment).//9.00E−24//64aa//36%//P36514
TESTI4001561//1-acyl-sn-glycerol-3-phosphate acyltransferase gamma (EC
2.3.1.51) (1-AGP acyltransferase 3) (1-AGPAT 3) (Lysophosphatidic acid
acyltransferase-gamma) (LPAAT-gamma) (1-acylglycerol-3-phosphate 0-
acyltransferase 3).//0//319aa//93%//Q9NRZ7
TESTI4001665
TESTI4001923
TESTI4002290
TESTI4002491//Beta-soluble NSF attachment protein (SNAP-beta) (N-
ethylmaleimide-sensitive factor attachment protein, beta) (Brain protein
I47) (Fragment).//1.00E−52//99aa//93%//P28663
TESTI4002552//Sodium/potassium-transporting ATPase alpha-4 chain (EC
3.6.3.9) (Sodium pump 4) (Na+/K+ ATPase 4) (Fragment).//0//505aa//94%//
Q13733
TESTI4002647
TESTI4002703
TESTI4002754
TESTI4002878
TESTI4004200
TESTI4005628
TESTI4005805
TESTI4005857
TESTI4005961
TESTI4006053
TESTI4006079//MUF1 protein; likely ortholog of mouse MUF1; elongin BC-
interacting leucine-rich repeat protein [Homo sapiens]//0//365aa//80%//
NM_006369
TESTI4006112
TESTI4006137
TESTI4006148//putative NADH oxidoreductase complex I subunit//2.00E−18//
40aa//56%//AAD37863.
TESTI4006219
TESTI4006326
TESTI4006393//neural specific sr protein NSSR 2 [Mus musculus]//7.00E−19//
70aa//80%//BAA35093
TESTI4006412
TESTI4006420//SH3-domain binding protein 5 (BTK-associated); SH3 binding
protein [Homo sapiens]//8.00E−25//61aa//41%//NM_004844
TESTI4006546//colon cancer antigen NY-CO-45 [Homo sapiens].//0//723aa//
99%//AAC18034
TESTI4006802//mesothelin; megakaryocyte potentiating factor [Mus musculus]//
2.00E−06//92aa//23%//NM_018857
TESTI4006819//Alpha enolase (EC 4.2.1.11) (2-phospho-D-glycerate hydrolyase)
(NON-neural enolase) (NNE) (Phosphopyruvate hydratase).//1.00E−33//
72aa//66%//P06733
TESTI4007064
TESTI4007163//Sodium- and chloride-dependent creatine transporter 2 (CT2)
(Fragment).//2.00E−92//153aa//84%//P53796
TESTI4007203
TESTI4007239
TESTI4007373
TESTI4007382
TESTI4007404
TESTI4007489
TESTI4007775
TESTI4007778//Alpha-actinin 3 (Alpha actinin skeletal muscle isoform 3) (F-
actin cross linking protein).//0//853aa//94%//Q08043
TESTI4007799
TESTI4007810//DNA ligase III (EC 6.5.1.1) (Polydeoxyribonucleotide synthase
[ATP]).//1.00E−112//197aa//86%//P49916
TESTI4008007
TESTI4008018//DAZ associated protein 2; KIAA0058 gene product [Homo
sapiens]//6.00E−41//82aa//75%//NM_014764
TESTI4008050//Translocation protein SEC63 homolog.//1.00E−175//314aa//
82%//Q9UGP8
TESTI4008219
TESTI4008401
TESTI4008429//Probable cation-transporting ATPase 2 (EC 3.6.3.—) (CGI-
152).//1.00E−136//249aa//94%//Q9HD20
TESTI4008573
TESTI4008797
TESTI4008816
TESTI4008935
TESTI4008993
TESTI4009022
TESTI4009034
TESTI4009123
TESTI4009160//Kinesin-like protein KIF2.//6.00E−06//39aa//37%//P28740
TESTI4009215
TESTI4009283
TESTI4009286//Homo sapiens HOTTL protein mRNA, complete cds//2.00E−78//
180aa//96%//AF078842
TESTI4009374//Apobec-1 complementation factor; APOBEC-1 stimulating protein;
apobec-1 complementation factor [Homo sapiens]//1.00E−120//203aa//68%//
NM_014576
TESTI4009406
TESTI4009457//p53-inducible p53DINP1 [Homo sapiens]//3.00E−80//140aa//
88%//NM_033285
TESTI4009563//testis specific ankyrin-like protein 1 [Homo sapiens]//1.00E−140
//239aa//94%//NM_017844
TESTI4009608//putative T1/ST2 receptor binding protein [Homo sapiens]//
1.00E−41//125aa//57%//NP_006849
TESTI4009638
TESTI4009881//Dynein heavy chain, cytosolic (DYHC) (Cytoplasmic dynein heavy
chain).//5.00E−30//176aa//21%//Q9JHU4
TESTI4010211
TESTI4010377
TESTI4010713
TESTI4010789
TESTI4010817
TESTI4010831//yeast Sec31p homolog; ABP125 [Homo sapiens]//0//780aa//
81%//NM_016211
TESTI4010851//Probable ubiquitin carboxyl-terminal hydrolase FAF-X (EC
3.1.2.15) (Ubiquitin thiolesterase FAF-X) (Ubiquitin-specific processing
protease FAF-X) (Deubiquitinating enzyme FAF-X) (Fat facets protein related,
X-linked) (Ubiquitin-specific protease 9, X chromosome).//2.00E−67//213aa//
25%//Q93008
TESTI4010928
TESTI4011118
TESTI4011161
TESTI4011246
TESTI4011484//Sec23-interacting protein p125 [Homo sapiens]//0//387aa//
52%//NM_007190
TESTI4011505
TESTI4011745//WD-repeat protein 9 (Fragment).//0//674aa//82%//Q9NSI6
TESTI4011956//Ciliary dynein heavy chain (Axonemal dynein heavy chain)
(Dynein heavy chain 9).//1.00E−170//340aa//39%//Q9NYC9
TESTI4012086
TESTI4012329
TESTI4012406//Apolipoprotein(A) (EC 3.4.21.—) (Apo(A)) (LP(A)) (Fragment).//
2.00E−25//50aa//79%//P14417
TESTI4012448//Stromelysin-3 precursor (EC 3.4.24.—) (Matrix
metalloproteinase-11) (MMP-11) (ST3) (SL-3).//0//375aa//99%//P24347
TESTI4012505//Tumor suppressor p53-binding protein 2 (p53-binding protein 2)
(53BP2) (Bcl2-binding protein) (Bbp).//1.00E−81//220aa//34%//Q13625
TESTI4012556
TESTI4012679//Homo sapiens cryptochrome 1 (photolyase-like) (CRY1), mRNA//
0//330aa//97%//NM_004075
TESTI4012702
TESTI4013369//ATP synthase lipid-binding protein, mitochondrial precursor
(EC 3.6.1.34) (ATP synthase proteolipid P3) (ATPase protein 9) (ATPase
subunit C).//7.00E−60//119aa//83%//P48201
TESTI4013667
TESTI4013675
TESTI4013685
TESTI4013735
TESTI4013817//novel AMP-binding enzyme similar to acetyl-coenzyme A
synthethase (acetate-coA ligase)//8.00E−38//99aa//100%//CAB75500
TESTI4013830//Integral membrane glycoprotein gp210 precursor.//0//652aa//
41%//P11654
TESTI4013924//Intracellular protein transport protein USO1.//8.00E−20//
125aa//20%//P25386
TESTI4014159
TESTI4014175//Chromodomain helicase-DNA-binding protein 3 (CHD-3) (Mi-2
autoantigen 240 kDa protein) (Mi2-alpha).//0//410aa//75%//Q12873
TESTI4014306
TESTI4014392
TESTI4014445
TESTI4014694
TESTI4014818//AD-012 protein [Homo sapiens]//1.00E−123//217aa//70%//
NM_018449
TESTI4014924//selective hybridizing clone [Mus musculus]//0//1153aa//
92%//NM_011370
TESTI4015263
TESTI4015293
TESTI4015471
TESTI4015600
TESTI4015646
TESTI4015681
TESTI4015688
TESTI4016110//DnaJ homolog subfamily B member 8 (mDJ6).//1.00E−91//165aa//
71%//Q9QYI7
TESTI4016238
TESTI4016551
TESTI4016812
TESTI4016822//Protein phosphatase inhibitor 2 (IPP-2).//9.00E−72//133aa//
83%//P41236
TESTI4016882
TESTI4016925//Dynein beta chain, ciliary.//0//533aa//34%//P39057
TESTI4017001
TESTI4017137
TESTI4017254
TESTI4017543//ubinuclein 1 [Homo sapiens]//1.00E−124//286aa//38%//
NM_016936
TESTI4017575
TESTI4017848
TESTI4017901//alpha-1A-adrenergic receptor, isoform 2; adrenergic, alpha-
1A-, receptor; adrenergic, alpha-1C-, receptor; alpha 1A-adrenoceptor [Homo
sapiens]//9.00E−21//51aa//72%//NM_033303
TESTI4017961
TESTI4018152//protein tyrosine phosphatase, non-receptor type 13 [Mus
musculus]//3.00E−18//130aa//33%//NP_035334.
TESTI4018208//MYOSINIC HEAVY CHAIN.//6.10E−07//112aa//40%//P10569
TESTI4018382
TESTI4018555
TESTI4018806
TESTI4018835//Potential phospholipid-transporting ATPase IK (EC 3.6.3.13)
(Fragment).//0//514aa//88%//060423
TESTI4018881//early endosome antigen 1, 162 kD; early endosome-associated
protein [Homo sapiens]//2.00E−14//101aa//22%//NM_003566
TESTI4018886//M-protein, striated muscle.//4.00E−81//146aa//46%//Q02173
TESTI4019140//Mi-2 histone deacetylase complex protein 66 [Xenopus laevis]//
2.00E−98//410aa//71%//AAD55392
TESTI4019299
TESTI4019417
TESTI4019566//Dosage compensation regulator (Male-less protein) (No action
potential protein).//8.00E−49//165aa//29%//P24785
TESTI4019843//Rattus norvegicus huntingtin-associated protein interacting
protein (duo) (Hapip), mRNA.//0//698aa//91%//NM_032062
TESTI4020092//Laminin alpha-2 chain precursor (Laminin M chain) (Merosin
heavy chain).//3.00E−40//74aa//96%//P24043
TESTI4020102
TESTI4020806
TESTI4020920
TESTI4021294
TESTI4021456
TESTI4021478//Potential phospholipid-transporting ATPase IS (EC 3.6.3.13)
(Fragment).//0//433aa//54%//P98196
TESTI4021491
TESTI4022716//RNA helicase [Homo sapiens]//0//817aa//95%//NM_014314
TESTI4022873//Dynein gamma chain, flagellar outer arm.//3.00E−09//106aa//
19%//Q39575
TESTI4022936
TESTI4023546//Sialidase (EC 3.2.1.18) (Neuraminidase) (NA) (Major surface
antigen).//6.00E−32//134aa//23%//P23253
TESTI4023555
TESTI4023722
TESTI4023762//Trichohyalin.//5.00E−12//94aa//22%//P37709
TESTI4023942
TESTI4024344
TESTI4024420//multidomain presynaptic cytomatrix protein Piccolo [Rattus
norvegicus]//0//789aa//82%//NM_020098
TESTI4024874
TESTI4024890
TESTI4024907
TESTI4025731
TESTI4025797
TESTI4025920//B29 protein [Homo sapiens]//2.00E−34//73aa//38%//NM_031939
TESTI4026079
TESTI4026192
TESTI4026295
TESTI4026456
TESTI4026510//RNA helicase [Homo sapiens]//0//445aa//89%//NM_016130
TESTI4026524//Chromodomain helicase-DNA-binding protein 4 (CHD-4) (Mi-2
autoantigen 218 kDa protein) (Mi2-beta).//0//388aa//59%//Q14839
TESTI4026700
TESTI4026762
TESTI4026785
TESTI4027516
TESTI4027557//Galectin-9 (HOM-HD-21) (Ecalectin).//1.00E−176//306aa//
86%//000182
TESTI4027821
TESTI4028059//6-phosphofructokinase, muscle type (EC 2.7.1.11)
(Phosphofructokinase 1) (Phosphohexokinase) (Phosphofructo-1-kinase isozyme
A) (PFK-A).//0//450aa//96%//P08237
TESTI4028062
TESTI4028429//Eppin precursor.//2.00E−32//61aa//76%//O95925
TESTI4028612
TESTI4028809
TESTI4028823//Niemann-Pick C1 protein precursor.//6.00E−22//127aa//22%//
P56941
TESTI4028880//Glucose transporter type 3, brain.//0//436aa//88%//P11169
TESTI4028983
TESTI4029370
TESTI4029671
TESTI4029836//Potential phospholipid-transporting ATPase IB (EC 3.6.3.13).//
0//888aa//93%//P98200
TESTI4030069//fer-1 (C. elegans)-like 3 (myoferlin); fer-1 (C. elegans)-like
3 [Homo sapiens]//4.00E−22//64aa//38%//NM_013451
TESTI4030159
TESTI4030505
TESTI4030603
TESTI4030669
TESTI4032895
TESTI4033433
TESTI4033690
TESTI4034172
TESTI4034212
TESTI4034432
TESTI4034632//polypeptide N-acetylgalactosaminyltransferase 9; UDP-GalNAc:
polypeptide N-acetylgalactosaminyltransferase 9; GalNAc transferase 9;
protein-UDP acetylgalactosaminyltransferase 9 [Homo sapiens]//1.00E−113//
182aa//60%//NM_021808
TESTI4034912//Intracellular protein transport protein US01.//6.00E−38//
219aa//21%//P25386
TESTI4035063//Restin (Cytoplasmic linker protein-170 alpha-2) (CLIP-170)
(Reed-Sternberg intermediate filament associated protein).//1.00E−17//
72aa//27%//P30622
TESTI4035065
TESTI4035498//Septin-like protein KIAA0202 (Fragment).//7.00E−58//112aa//
49%//Q92599
TESTI4035602
TESTI4035637
TESTI4035649
TESTI4036042
TESTI4036909//Regulator of nonsense transcripts 1 homolog.//9.00E−50//
140aa//32%//Q9FJR0
TESTI4037066
TESTI4037156//WHSC2 protein [Homo sapiens]//0//425aa//80%//NM_005663
TESTI4037188
TESTI4037244
TESTI4037727//Dynein beta chain, ciliary.//0//573aa//73%//P39057
TESTI4038156
TESTI4038223
TESTI4038258
TESTI4038339
TESTI4038492
TESTI4038818
TESTI4039038
TESTI4039086
TESTI4039659//DnaJ homolog subfamily B member 8 (mDJ6).//1.00E−91//165aa//
71%//Q9QY17
TESTI4040363//Surfeit locus protein 5.//3.00E−62//120aa//100%//Q15528
TESTI4040800
TESTI4040939
TESTI4040956
TESTI4041053
TESTI4041099
TESTI4041143
TESTI4041519
TESTI4041624
TESTI4041903
TESTI4041954
TESTI4042098
TESTI4042444
TESTI4042711
TESTI4043129
TESTI4043203
TESTI4043551
TESTI4043947
TESTI4044035
TESTI4044084
TESTI4044123
TESTI4044186//leucine-rich, glioma inactivated 1 [Mus musculus]//6.00E−65//
110aa//60%//NM_020278
TESTI4044234
TESTI4044296
TESTI4044682
TESTI4045312
TESTI4046253
TESTI4046282
TESTI4046487//plexin 1 [Mus musculus]//0//433aa//97%//NM_008881
TESTI4046819//Glucoamylase S1/S2 precursor (EC 3.2.1.3) (Glucan 1,4-alpha-
glucosidase) (1,4-alpha-D-glucan glucohydrolase).//4.00E−12//134aa//21%//
P08640
TESTI4046884
TESTI4047069
THYMU1000496//KINESIN-LIKE PROTEIN KIF1C.//6.40E−61//210aa//53%//O43896
THYMU1000600
THYMU2000932
THYMU2001053
THYMU2001090
THYMU2003397
THYMU2003632
THYMU2003760
THYMU2004693
THYMU2005003
THYMU2005190
THYMU2005303//T-CELL SURFACE GLYCOPROTEIN CD8 ALPHA CHAIN PRECURSOR (T-
LYMPHOCYTE DIFFERENTIATION ANTIGEN T8/LEU-2).//4.2E−56//111aa//100%//
P01732
THYMU2005321
THYMU2006420//TRANSCRIPTION FACTOR-LIKE PROTEIN MRGX (KIAA0026).//2.00E−129
//268aa//92%//Q15014
THYMU2007060//Mus musculus Cdc42 GTPase-activating protein mRNA, complete
cds.//1.50E−37//270aa//40%//AF151363
THYMU2007179
THYMU2007658
THYMU2008282
THYMU2008725//PROTEIN-TYROSINE PHOSPHATASE BETA PRECURSOR (EC 3.1.3.48) (R-
PTP-BETA).//5.90E−192//358aa//98%//P23467
THYMU2009134
THYMU2009157//Mus musculus MRPS18b mRNA for mitochondrial ribosomal protein
S18b, complete cds.//5.00E−38//97aa//77%//AB049954
THYMU2009425//OLFACTORY RECEPTOR-LIKE PROTEIN HGMP07J.//4.90E−46//1 73aa//
53%//P30954
THYMU2011548//olfactory receptor 67 [Mus musculus]//2.50E−56//307aa//
39%//NP_038647
THYMU2011736//latent transforming growth factor beta binding protein 3//0//
200aa//99%//NP_066548
THYMU2013386//COTE1 PROTEIN.//2.50E−25//269aa//28%//P81408
THYMU2014353
THYMU2016204
THYMU2016523
THYMU2019210//HLA CLASS I HISTOCOMPATIBILITY ANTIGEN, B-40 B*4002 ALPHA
CHAIN PRECURSOR.//2.1E−195//248aa//100%//Q04826
THYMU2019587
THYMU2023711//Homo sapiens mRNA for immunoglobulin lambda heavy chain.//
2.50E−233//477aa//89%//Y14737
THYMU2023967
THYMU2025707
THYMU2027497//5-HYDROXYTRYPTAMINE 3 RECEPTOR PRECURSOR (5-HT-3) (SEROTONIN-
GATED ION CHANNEL RECEPTOR) (5-HT3R).//2E−10//186aa//24%//P46098
THYMU2027695//Ig gamma-1 chain C region.//1.00E−169//295aa//78%//P01857
THYMU2027734//Homo sapiens SA hypertension-associated homolog (rat) (SAH),
mRNA.//2.00E−39//72aa//42%//NM_005622
THYMU2028978
THYMU2029676
THYMU2029688
THYMU2030068
THYMU2030226
THYMU2030264
THYMU2030637
THYMU2030796
THYMU2031046//Copine III.//4.00E−28//60aa//75%//O75131
THYMU2031218
THYMU2031258//Homo sapiens oxysterol-binding protein-related protein (ORP1)
mRNA, complete cds.//4.0E−45//125aa//64%//AF274714
THYMU2031341
THYMU2031368
THYMU2031579
THYMU2031847
THYMU2031890
THYMU2032014//src homology 3 domain-containing protein HIP-55; HIP-55
protein [Homo sapiens]//2.00E−84//147aa//90%//NM_014063
THYMU2032035
THYMU2032080
THYMU2032358
THYMU2032437
THYMU2032655
THYMU2032696
THYMU2032825//Mus musculus mRNA for Drctnnb1a, complete cds.//2.3E−74//
202aa//71%//AB030242
THYMU2033070
THYMU2033079//ATP-binding cassette protein [Mus musculus].//2.00E−53//
105aa//91%//AAF31421
THYMU2033104//nuclear prelamin A recognition factor, isoform a [Homo
sapiens]//5.00E−34//111aa//47%//NP_036468
THYMU2033308
THYMU2033787
THYMU2033816
THYMU2034314
THYMU2034374//Homo sapiens MAID protein mRNA, complete cds.//1.5E−75//
146aa//100%//AF113535
THYMU2034647
THYMU2035064
THYMU2035101
THYMU2035319//Homo sapiens RNA-binding region (RNP1, RRM) containing 2
(RNPC2)//0//354aa//81%//NM_004902
THYMU2035388
THYMU2035400
THYMU2035735//Oryctolagus cuniculus sarcolemmal associated protein-3 mRNA,
complete cds.//3.6E−154//350aa//90%//U21157
THYMU2036058
THYMU2036085
THYMU2036252
THYMU2036265
THYMU2036459//240 KDA PROTEIN OF ROD PHOTORECEPTOR CNG-CHANNEL [CONTAINS:
GLUTAMIC ACID-RICH PROTEIN (GARP); CYCLIC-NUCLEOTIDE-GATED CATION CHANNEL 4
(CNG CHANNEL 4) (CNG-4) (CYCLIC NUCLEOTIDE-GATED CATION CHANNEL MODULATORY
SUBUNIT)].//1.40E−13//527aa//24%//Q28181
THYMU2036653
THYMU2037081
THYMU2037208
THYMU2037226
THYMU2037233//RNA polymerase I transcription factor RRN3 [Homo sapiens]//
1.00E−71//143aa//95%//NP_060897
THYMU2037348
THYMU2037965
THYMU2038189
THYMU2038301//Homo sapiens mRNA for PRP8 protein, complete cds.//3.90E−52//
112aa//98%//AB007510
THYMU2038369//Mus musculus GTRGE022 (Gtrgeo22) mRNA, complete cds.//1.10E−111
//262aa//83%//AF303106
THYMU2038615
THYMU2038636
THYMU2038739
THYMU2038772
THYMU2038797//B locus C type Lectin [Gallus gallus]//2.90E−15//147aa//
34%//CAA18961
THYMU2039305//70 KDA WD-REPEAT TUMOR-SPECIFIC ANTIGEN (FRAGMENT).//6.90E−40
//98aa//83%//O35828
THYMU2039315//Caenorhabditis elegans LIN-9L (lin-9) mRNA, complete cds.//
8.70E−66//444aa//34%//AF269693
THYMU2039350
THYMU2039411
THYMU2039780
THYMU2039989
THYMU2040140
THYMU2040412
THYMU2040824
THYMU2040975//PTB-ASSOCIATED SPLICING FACTOR (PSF).//1.30E−08//119aa//
36%//P23246
THYMU2041007
THYMU2041015//Monocarboxylate transporter 8 (MCT 8) (X-linked PEST-
containing transporter) (MCT 7).//1.00E−132//230aa//54%//P36021
THYMU2041252
THYMU3000028//Rat Tamm-Horsfall protein mRNA, complete cds.//1.3E−21//
253aa//28%//M63510
THYMU3000036
THYMU3000133
THYMU3000655
THYMU3000826
THYMU3001083//Tubulin epsilon chain (Epsilon tubulin).//5.00E−26//58aa//
98%//Q9UJT0
THYMU3001234//Dynamin 2 (EC 3.6.1.50) (Dynamin UDNM).//1.00E−56//108aa//
90%//P39054
THYMU3001379//116 kDa U5 small nuclear ribonucleoprotein component (U5
snRNP-specific protein, 116 kDa) (U5-116 kDa).//0//492aa//100%//Q15029
THYMU3001472
THYMU3001991//ART-4 protein [Homo sapiens]//2.00E−46//88aa//97%//
NM_014062
THYMU3002452
THYMU3002661
THYMU3003212//Saccharomyces cerevisiae TAD2 gene for tRNA-specific
adenosine-34 deaminase subunit Tad2p.//1.10E−21//135aa//40%//AJ242667
THYMU3003309//putative tumor antigen [Homo sapiens]//2.00E−52//105aa//
66%//NM_018666
THYMU3003763
THYMU3004157//peroxisomal acyl-CoA thioesterase [Homo sapiens]//3.00E−44//
85aa//82%//NM_005469
THYMU3004835//Probable beta-1,3-galactosyltransferase 8 (EC 2.4.1.—) (Beta-
1,3-GalTase 8) (Beta3Gal-T8) (b3Gal-T8) (UDP-galactose:beta-N-
acetylglucosamine beta-1,3-galactosyltransferase 8) (UDP-Gal:beta-GlcNAc
beta-1,3-galactosyltransferase 8) (Beta-3-Gx-T8).//2.00E−78//146aa//43%//
Q9Y2A9
THYMU3004866//TPA inducible gene-1; TPA inducible protein [Homo sapiens]//
3.00E−47//93aa//86%//NM_015889
THYMU3005696
THYMU3006118//molybdenum cofactor synthesis 2 [Homo sapiens]//3.00E−60//
112aa//100%//NM_004531
THYMU3006132
THYMU3006168
THYMU3006172//membrane bound C2 domain containing protein [Rattus
norvegicus]//1.00E−145//460aa//52%//NP_058945
THYMU3006371
THYMU3006485
THYMU3006811//ATP-binding cassette, sub-family A, member 7, isoform a//
3.00E−11//82aa//41%//NP_061985
THYMU3006963
THYMU3007137//Interleukin-16 precursor (IL-16) (Lymphocyte chemoattractant
factor) (LCF).//0//528aa//83%//Q14005
THYMU3007368
THYMU3007845
THYMU3008171
THYMU3008436//6-phosphofructokinase, muscle type (EC 2.7.1.11)
(Phosphofructokinase 1) (Phosphohexokinase) (Phosphofructo-1-kinase isozyme
A) (PFK-A).//0//764aa//98%//P08237
THYMU3009255
TKIDN2000701//ankyrin G//1.6E−90//178aa//100%//AAA64834
TKIDN2002424
TKIDN2002632
TKIDN2003044
TKIDN2004386
TKIDN2005934
TKIDN2005947
TKIDN2006525
TKIDN2006852//Homo sapiens cytosolic phospholipase A2 gamma (cPLA2 gamma)
mRNA, complete cds.//4.3E−103//192aa//100%//AF065214
TKIDN2007667
TKIDN2009092
TKIDN2009641
TKIDN2009889
TKIDN2010934
TKIDN2012824
TKIDN2013287
TKIDN2014757
TKIDN2014771
TKIDN2015263
TKIDN2015788
TKIDN2016309
TKIDN2019116
TLIVE2000023
TLIVE2001327//Human DOCK180 protein mRNA, complete cds.//0//961aa//63%//
D50857
TLIVE2001828
TLIVE2001927
TLIVE2002336//ectonucleotide pyrophosphatase/phosphodiesterase 5 [Mus
musculus]//7.00E−69//144aa//36%//NM_032003
TLIVE2002338
TLIVE2002690
TLIVE2003197
TLIVE2003225//CUB and Sushi multiple domains 1 [Homo sapiens]//1.00E−129//
199aa//58%//NM_033225
TLIVE2003381//taste receptor, type 1, member 3; saccharin preference [Mus
musculus]//8.00E−65//112aa//79%//NM_031872
TLIVE2003970
TLIVE2004110
TLIVE2004320//Homo sapiens PC2-glutamine-rich-associated protein (PCQAP)
mRNA, complete cds.//4.7E−201//368aa//99%//AF328769
TLIVE2004601
TLIVE2005180
TLIVE2006236
TLIVE2006529
TLIVE2007132
TLIVE2007528
TLIVE2007816
TLIVE2008083
TLIVE2008229//SIGNAL RECOGNITION PARTICLE 68 KDA PROTEIN (SRP68).//1.00E−299
//506aa//96%//Q00004
TLIVE2009541
TOVAR2000649
TOVAR2001281
TOVAR2001730
TOVAR2002247//Homo sapiens partial partial mRNA for NICE-4 protein, clone
3114f17.//1.0E−117//218aa//100%//AJ243670
TOVAR2002549
TRACH1000205
TRACH2001443
TRACH2001549//Homo sapiens mRNA for neuropathy target esterase.//1.10E−94//
295aa//65%//AJ004832
TRACH2001684
TRACH2003070
TRACH2004170
TRACH2005066
TRACH2005811
TRACH2006049
TRACH2006387//P2Y PURINOCEPTOR 1 (ATP RECEPTOR) (P2Y1) (PURINERGIC
RECEPTOR).//2E−56//307aa//36%//P49650
TRACH2007059//Folate hydrolase (Prostate-specific membrane antigen 1).//
2.00E−37//127aa//26%//Q04609
TRACH2007834
TRACH2008300
TRACH2009310//PUTATIVE SERINE/THREONINE-PROTEIN KINASE D1044.3 IN CHROMOSOME
III (EC 2.7.1.—).//9.40E−85//407aa//38%//P41951
TRACH2019248
TRACH2019473
TRACH2020525
TRACH2021398
TRACH2021964
TRACH2022042
TRACH2022425//Ig alpha-1 chain C region.//0//319aa//91%//P01876
TRACH2022553//Human germline IgD-chain gene, C-region, second domain of
membrane terminus.//1.70E−234//429aa//99%//K02882
TRACH2022649//Ig gamma-1 chain C region.//0//315aa//95%//P01857
TRACH2023299//growth factor receptor bound protein 2-associated protein 2
[Mus musculus]//5.00E−40//77aa//58%//NM_010248
TRACH2023306
TRACH2025344
TRACH2025507//tumor suppressing subtransferable candidate 1; tumor-
supressing STF cDNA 1 [Homo sapiens]//4.00E−48//87aa//74%//NM_003310
TRACH2025535//evectin-2 [Mus musculus]//2.00E−75//230aa//90%//AAF01332
TRACH2025749
TRACH2025911
TRACH2025932
TRACH3000014
TRACH3000342
TRACH3000558//CREB-BINDING PROTEIN.//1.9E−90//120aa//100%//Q92793
TRACH3000586
TRACH3000926//cardiac morphogenesis [Mus musculus]//0//417aa//63%//
NM_011724
TRACH3001427//p47 [Homo sapiens]//2.00E−85//167aa//49%//NM_016143
TRACH3002064
TRACH3002168//Cell surface glycoprotein MUC18 precursor (Melanoma-associated
antigen MUC18) (Melanoma-associated antigen A32) (S-endo 1 endothelial-
associated antigen) (CD146 antigen) (Melanoma adhesion molecule).//0//
341aa//94%//P43121
TRACH3002192
TRACH3002650
TRACH3002866
TRACH3002871
TRACH3003379
TRACH3004068
TRACH3004537
TRACH3004721//80 kda MCM3-associated protein (GANP protein).//0//474aa//
77%//060318
TRACH3004786//Claudin-4 (Clostridium perfringens enterotoxin receptor) (CPE-
receptor) (CPE-R).//2.00E−90//162aa//77%//014493
TRACH3004840
TRACH3005294
TRACH3005479
TRACH3005549//Ig heavy chain V region IR2 precursor.//4.00E−47//89aa//
61%//P01805
TRACH3006038
TRACH3006149
TRACH3006228
TRACH3006412//Homo sapiens COP9 constitutive photomorphogenic homolog
subunit 7B//3.00E−57//105aa//99%//NM_022730
TRACH3006470
TRACH3006889
TRACH3007391
TRACH3007479//Nedd-4-like ubiquitin-protein ligase; WW domain-containing
protein 2 [Homo sapiens]//0//320aa//93%//NM_007014
TRACH3008093
TRACH3008535
TRACH3008629//Cadherin-related tumor suppressor homolog precursor (Fat
protein homolog).//6.00E−36//143aa//28%//Q14517
TRACH3008713//Beta-soluble NSF attachment protein (SNAP-beta) (N-
ethylmaleimide-sensitive factor attachment protein, beta) (Brain protein
I47) (Fragment).//4.00E−52//98aa//92%//P28663
TRACH3009455//Phosphatidylinositol 3-kinase regulatory alpha subunit (PI3-
kinase P85-alpha subunit) (PtdIns-3-kinase P85-alpha) (PI3K).//0//386aa//
95%//P27986
TRACH3034731//Ras association (RaIGDS/AF-6) domain family 2//7.00E−56//
320aa//40%//NP_055552
TRACH3034762
TRACH3035199//antigen identified by monoclonal antibody MRC OX-2 receptor
[Rattus norvegicus]//1.00E−86//170aa//51%//NM_023953
TRACH3035235
TRACH3035482
TRACH3035526//Ig alpha-2 chain C region.//0//324aa//95%//P01877
TRACH3036193//Genome polyprotein [Contains: Coat proteins VP1 TO VP4; Core
proteins P2A TO P2C, P3A; Genome-linked protein VPG; Picornain 3C (EC
3.4.22.28) (Protease 3C) (P3C); RNA-directed RNA polymerase P3D (EC
2.7.7.48)].//0//1073aa//69%//Q82122
TRACH3036207
TRACH3036309
TRACH3036456
TRACH3036609//J kappa-recombination signal binding protein (RBP-J kappa).//
1.00E−158//271aa//89%//P31266
TSTOM1000135
TSTOM2000442//Ig gamma-1 chain C region.//1.00E−168//292aa//77%//P01857
TSTOM2000553//SYNAPTOTAGMIN IV.//3.00E−08//150aa//28%//P40749
TSTOM2002672
TUTER1000122
TUTER2000425//zinc finger protein SBZF3 [Homo sapiens]//4.00E−36//74aa//
81%//NM_020394
TUTER2000904//Unc-119 protein homolog (Retinal protein 4) (RRG4).//7.00E−72
//129aa//70%//Q62885
TUTER2000916
TUTER2001387
TUTER2002729//D6MM5E protein [Mus musculus]//1.00E−107//191aa//68%//
NM_033079
UTERU1000024
UTERU1000031//G. gallus mRNA for tom-1B protein.//2.1E−149//535aa//59%//
Y08741
UTERU1000148
UTERU1000249
UTERU1000337//Putative protein phosphatase 2C (EC 3.1.3.16) (PP2C).//1.00E−156
//271aa//94%//P49593
UTERU1000339
UTERU2000649
UTERU2001409
UTERU2002410
UTERU2002841
UTERU2004688
UTERU2004929
UTERU2005004
UTERU2005621//CDC14 homolog B, isoform 2 [Homo sapiens]//0//423aa//94%//
NM_033331
UTERU2006115//ALPHA-ADAPTIN A (CLATHRIN ASSEMBLY PROTEIN COMPLEX 2 ALPHA-A
LARGE CHAIN) (100 KDA COATED VESICLE PROTEIN A) (PLASMA MEMBRANE ADAPTOR
HA2/AP2 ADAPTIN ALPHA A SUBUNIT).//9.0E−141//268aa//99%//P17426
UTERU2006137
UTERU2006568
UTERU2007444
UTERU2007520
UTERU2007724//Calponin H2, smooth muscle (Neutral calponin).//1.00E−144//
253aa//86%//Q99439
UTERU2008347//Chlamydomonas reinhardtii vegetative cell wall protein gp1
(GP1) gene, complete cds.//1.0E−19//199aa//30%//AF309494
UTERU2014678
UTERU2017762//plexin B1; KIAA0407 protein; plexin 5 [Homo sapiens]//0//
383aa//60%//NM_002673
UTERU2019491//Homo sapiens mRNA for 41-kDa phosphoribosylpyrophosphate
synthetase-associated protein, complete cds.//4.30E−48//101aa//100%//
AB007851
UTERU2019681
UTERU2019706//T-COMPLEX PROTEIN 1, GAMMA SUBUNIT (TCP-1-GAMMA) (CCT-
GAMMA).//9.80E−273//426aa//99%//P49368
UTERU2019940//mitochondrial ribosomal protein L30 [Homo sapiens]//2.00E−44
//82aa//97%//NM_016503
UTERU2020491
UTERU2020718
UTERU2021163
UTERU2021380
UTERU2022020
UTERU2022981
UTERU2023039
UTERU2023175
UTERU2023651
UTERU2023712
UTERU2024002
UTERU2024656
UTERU2025025//High affinity nerve growth factor receptor precursor (EC
2.7.1.112) (TRK1 transforming tyrosine kinase protein) (p140-TrkA) (Trk-A).//
0//479aa//95%//P04629
UTERU2025645
UTERU2025891
UTERU2026025//SPLICING FACTOR, ARGININE/SERINE-RICH 2 (SPLICING FACTOR SC35)
(SC-35) (SPLICING COMPONENT, 35 KDA) (PR264 PROTEIN).//8.00E−30//61aa//
100%//P30352
UTERU2026090//Cartilage-associated protein precursor.//1.00E−180//309aa//
87%//O75718
UTERU2026203//phosphoinositide phosphatase SAC1 [Rattus norvegicus].//
1.00E−107//221aa//95%//AAG29810
UTERU2027591//calcium-activated potassium channel//7.8E−33//79aa//94%//
AAA50216
UTERU2029953
UTERU2030213
UTERU2030280
UTERU2031084
UTERU2031268//NY-REN-25 antigen [Homo sapiens].//1.00E−41//330aa//49%//
AAD42869
UTERU2031521
UTERU2031703
UTERU2031851
UTERU2033375
UTERU2033382
UTERU2035114
UTERU2035323
UTERU2035328//Homo sapiens putative transcription factor CA150 mRNA,
complete cds.//1.80E−271//796aa//70%//AF017789
UTERU2035331
UTERU2035452//NG3 [Homo sapiens]//1.00E−136//150aa//99%//AAB47494
UTERU2035469//Mus musculus microfibril-associated glycoprotein-2 (Magp2)
mRNA, complete cds.//1.2E−52//164aa//66%//AF180805
UTERU2035503
UTERU2035745//MYOSIN IA HEAVY CHAIN (MYOSIN-LIKE PROTEIN ABMA).//1.30E−11//
101aa//31%//P22467
UTERU2036089//SH3-BINDING PROTEIN 3BP-1.//4.1E−168//369aa//86%//P55194
UTERU2037361
UTERU2037577
UTERU2038251
UTERU3000226
UTERU3000645//Claudin-4 (Clostridium perfringens enterotoxin receptor) (CPE-
receptor) (CPE-R).//3.00E−89//161aa//77%//O14493
UTERU3000665//Snf2-related CBP activator protein [Homo sapiens].//7.00E−59
//500aa//97%//NP_006653
UTERU3000828//116 kDa U5 small nuclear ribonucleoprotein component (U5
snRNP-specific protein, 116 kDa) (U5-116 kDa).//0//931aa//95%//Q15029
UTERU3000899//hTGN51 [Homo sapiens].//1.00E−101//281aa//72%//AAC39542
UTERU3001059//ABC1 protein homolog, mitochondrial precursor.//2.00E−99//
188aa//48%//Q92338
UTERU3001240//Adenylate cyclase, type IV (EC 4.6.1.1) (ATP pyrophosphate-
lyase) (Adenylyl cyclase).//1.00E−176//308aa//81%//P26770
UTERU3001542
UTERU3001571
UTERU3001572//Neuroblast differentiation associated protein AHNAK
(Desmoyokin) (Fragments).//6.00E−19//213aa//21%//Q09666
UTERU3001585//Cytochrome P450 4c3 (EC 1.14.—.—) (CYPIVC3).//1.00E−125//
230aa//49%//Q9VA27
UTERU3001652//64 KDA AUTOANTIGEN D1 (THYROID-ASSOCIATED OPHTHALMOPATHY
AUTOANTIGEN).//1.00E−219//416aa//99%//P29536
UTERU3001766
UTERU3001988//COATOMER EPSILON SUBUNIT (EPSILON-COAT PROTEIN) (EPSILON-
COP).//1.70E−126//159aa//94%//Q28104
UTERU3002209
UTERU3002218
UTERU3002383
UTERU3002667
UTERU3002731
UTERU3002768
UTERU3002786
UTERU3002993
UTERU3003116//ADAM 12 precursor (EC 3.4.24.—) (A disintegrin and
metalloproteinase domain 12) (Meltrin alpha).//1.00E−20//48aa//44%//
O43184
UTERU3003135//Splicing factor 3B subunit 2 (Spliceosome associated protein
145) (SAP 145) (SF3b150) (Pre-mRNA splicing factor SF3b 145 kDa subunit).//
6.00E−29//62aa//83%//Q13435
UTERU3003178//Kinesin light chain 2 (KLC 2).//0//312aa//88%//Q9H0B6
UTERU3003465
UTERU3003523
UTERU3003776
UTERU3004523
UTERU3004616
UTERU3004709
UTERU3004992//Aortic preferentially expressed protein 1 (APEG-1).//3.00E−61
//113aa//100%//Q15772
UTERU3005049
UTERU3005205
UTERU3005230
UTERU3005460
UTERU3005585//rhophilin-like protein [Homo sapiens]//0//380aa//91%//
NM_033103
UTERU3005907//PROTEIN-GLUTAMINE GAMMA-GLUTAMYLTRANSFERASE (EC 2.3.2.13)
(TISSUE TRANSGLUTAMINASE) (TGASE C) (TGC) (TGASE-H).//1.30E−75//152aa//
98%//P21980
UTERU3005970
UTERU3006008
UTERU3006308//SEMAPHORIN 4C PRECURSOR (SEMAPHORIN I)//1.00E−128//330aa//
86%//Q64151
UTERU3007134
UTERU3007419//Rattus norvegicus Ca2+-dependent activator protein (CAPS) mRNA,
complete cds.//0//1223aa//78%//U16802
UTERU3007640//N-ethylmaleimide-sensitive factor attachment protein, alpha;//
5.00E−54//110aa//87%//NP_003818
UTERU3007913
UTERU3008660
UTERU3008671//SPARC precursor (Secreted protein acidic and rich in cysteine)
(Osteonectin) (ON) (Basement membrane protein BM-40).//4.00E−25//49aa//
96%//P09486
UTERU3009259
UTERU3009490//LYSP100 protein (Lymphoid-restricted homolog of Sp100)
(Nuclear autoantigen Sp-140) (Speckled 140 kDa) (Nuclear body protein
Sp140).//4.00E−33//67aa//61%//Q13342
UTERU3009517
UTERU3009690//alpha-1A-adrenergic receptor, isoform 2; adrenergic, alpha-
1A-, receptor; adrenergic, alpha-1C-, receptor; alpha 1A-adrenoceptor [Homo
sapiens]//5.00E−16//41aa//67%//NM_033303
UTERU3009871//feminization 1 homolog a (C. elegans)//0//588aa//85%//
NP_034322
UTERU3009979//growth arrest-specific 6; AXL stimulatory factor [Homo
sapiens]//0//572aa//97%//NM_000820
UTERU3011063//Transmembrane 9 superfamily protein member 4.//0//402aa//
89%//Q92544
UTERU3015086
UTERU3015500//G protein-coupled receptor 49//1.00E−15//350aa//29%//
NP_003658
UTERU3016789//SH3 domain-binding protein 3BP-2.//1.00E−146//261aa//75%//
P78314
UTERU3018081
UTERU3018154
UTERU3018616
UTERU3018711
3NB692004724
ADRGL2000042//Homo sapiens CTCL tumor antigen se20-4 mRNA, complete cds.//
6.20E−143//269aa//100%//AF273046
ADRGL2000056
BLADE2000579
BLADE2006830
BRACE2002589
BRACE2003609//endothelial zinc finger protein induced by tumor necrosis
factor alpha [Homo sapiens]//0//310aa//67%//NM_021216
BRACE2009318
BRACE2011677
BRACE2029396
BRACE2037299
BRACE2039823//CDP-DIACYLGLYCEROL-INOSITOL 3-PHOSPHATIDYLTRANSFERASE (EC
2.7.8.11) (PHOSPHATIDYLINOSITOL SYNTHASE) (PTDINS SYNTHASE) (PI SYNTHASE).//
2.10E−79//154aa//100%//O14735
BRACE2039832
BRACE2043105
BRACE3001058//zinc finger protein 347; zinc finger 1111 [Homo sapiens]//0//
382aa//52%//NM_032584
BRACE3001113//Zinc finger protein 91 (Zinc finger protein HTF10) (HPF7).//
6.00E−92//226aa//26%//Q05481
BRACE3003026
BRACE3003053
BRACE3005107// // // // //
BRACE3009127//oxysterol binding protein 2; oxysterol binding protein-like 1
[Homo sapiens]//0//670aa//95%//NM_030758
BRACE3010076//Vigilin (High density lipoprotein-binding protein) (HDL-
binding protein).//0//464aa//92%//Q00341
BRACE3015829
BRACE3021148//DC12 protein [Homo sapiens]//1.00E−16//60aa//30%//
NM_020187
BRALZ2017844//HOMEOBOX PROTEIN CHOX-E (CHOX E) (FRAGMENT).//2.80E−59//
157aa//75%//Q91975
BRAMY2019111//POLYCYSTIN 2.//6.30E−18//204aa//27%//O35245
BRAMY2035070//Homo sapiens zinc finger 1111 mRNA, complete cds.//1.70E−213
//723aa//53%//AY029765
BRAMY2035449//Mus musculus zinc finger protein ZFP113 mRNA, complete cds.//
2.70E−95//356aa//44%//AF167320
BRAMY2035718//NUCLEAR FACTOR 1-B (NFI-B) (CCAAT BOX-BINDING TRANSCRIPTION
FACTOR) (CTF) (TGGCA-BINDING PROTEIN).//5.60E−228//418aa//97%//P97863
BRAMY2038516//PROBABLE PROTEIN DISULFIDE ISOMERASE P5 PRECURSOR (EC
5.3.4.1).//7.00E−237//434aa//99%//Q15084
BRAMY2039341//ZINC FINGER PROTEIN 135.//7.30E−84//227aa//62%//P52742
BRAMY2040159//Homo sapiens MRIP-1 mRNA, complete cds.//6.00E−234//345aa//
91%//AF359283
BRAMY2041434
BRAMY2045471//Homo sapiens CGI-89 protein mRNA, complete cds.//6.00E−45//
147aa//44%//CAB82308
BRAMY3004800//Huntingtin-associated protein-interacting protein (Duo
protein).//0//751aa//91%//O60229
BRAWH1000369//Homo sapiens putative DNA polymerase mRNA, partial cds.//
5.40E−150//199aa//97%//AF044578
BRAWH2006207//ZINC FINGER PROTEIN 91 (ZINC FINGER PROTEIN HTF10) (HPF7).//
8.90E−32//83aa//81%//Q05481
BRAWH2006395//transmembrane receptor UNC5H1//1.00E−141//292aa//88%//
AAB57678
BRAWH2008993
BRAWH2009393
BRAWH2010552
BRAWH3007441//CAT56 protein [Homo sapiens]//1.00E−44//78aa//84%//
NM_025263
BRAWH3009017//SEL-10 protein.//6.00E−38//73aa//61%//Q93794
BRCAN2002473//Tropomyosin, fibroblast isoform 2 (TM-2).//1.00E−114//
230aa//70%//P19354
BRCAN2002854//Human Hsp27 ERE-TATA-binding protein (HET) mRNA, complete
cds.//2.60E−21//240aa//35%//U72355
BRCAN2003070//ubiquitin carrier protein E2-C [Homo sapiens]//9.00E−51//
80aa//78%//NP_008950
BRCAN2014229
BRCOC2019841
BRHIP2002722
BRHIP2003272
BRHIP2005271//protein tyrosine phosphatase//7.00E−39//190aa//42%//
NP_006255
BRHIP2005724//Homo sapiens mRNA for NDRG4-B, complete cds.//3.10E−182//
301aa//98%//AB044944
BRHIP2006617//NUCLEAR AUTOANTIGENIC SPERM PROTEIN (NASP).//1.20E−156//
313aa//97%//P49321
BRHIP2008389//Drosophila melanogaster furry protein short isoform mRNA,
partial cds.//1.70E−29//179aa//44%//AF351187
BRHIP2012360//XPG_XENLA//4.00E−17//70aa//41%//P14629
BRHIP2017553//Zinc finger protein 184.//1.00E−164//272aa//49%//Q99676
BRHIP2026877//Tyrosine-protein kinase receptor TYRO3 precursor (EC
2.7.1.112) (Tyrosine-protein kinase RSE) (Tyrosine-protein kinase SKY)
(Tyrosine-protein kinase DTK) (Protein-tyrosine kinase byk).//3.00E−56//
104aa//80%//Q06418
BRHIP3000017
BRHIP3000240//Homo sapiens potassium channel beta 2 subunit (HKvbeta2.2)
mRNA, alternatively spliced, complete cds.//1.70E−172//347aa//95%//
AF044253
BRHIP3008314//sirtuin 2, isoform 2; silencing information regulator 2-like;
sir2-like 2; silent mating type information regulation 2, S. cerevisiae,
homolog 2; sir2-related protein type 2 [Homo sapiens]//2.00E−48//86aa//
98%//NM_030593
BRHIP3026052//Serine/threonine protein phosphatase 2A, 56 kDa regulatory
subunit, gamma isoform (PP2A, B subunit, B′ gamma isoform) (PP2A, B subunit,
B56 gamma isoform) (PP2A, B subunit, PR61 gamma isoform) (PP2A, B subunit, R5
gamma isoform).//0//424aa//83%//Q13362
BRSTN2013354//ETS-related protein PE-1 (ETS translocation variant 3)
(Fragment).//4.00E−61//109aa//84%//P41162
BRTHA2002133
BRTHA2002702
BRTHA2007060//EUKARYOTIC TRANSLATION INITIATION FACTOR 3 SUBUNIT 10 (EIF-3
THETA) (EUKARYOTIC TRANSLATION INITIATION FACTOR 3 LARGE SUBUNIT) (PNLA-
35).//0//963aa//74%//Q40554
BRTHA2010033
BRTHA2011321
BRTHA2013426
BRTHA2013610//deoxyguanosine kinase, putative [Arabidopsis thaliana]//
1.00E−142//360aa//78%//NP_565032*
BRTHA2016318//WTAP protein//9.00E−87//240aa//100%//CAC10188
BRTHA2017364//PUTATIVE ATP-DEPENDENT RNA HELICASE T26G10.1 IN CHROMOSOME
III.//1.10E−33//207aa//34%//P34580
BRTHA2017972//Homo sapiens MAD-related gene SMAD7 (SMAD7) mRNA, complete
cds.//5.30E−114//207aa//99%//AF010193
BRTHA2018011//EPITHIN (EC 3.4.21.—).//1.70E−53//242aa//45%//P56677
BRTHA2018443//POTENTIAL PHOSPHOLIPID-TRANSPORTING ATPASE IS (EC 3.6.1.—)
(FRAGMENT).//3.10E−195//581aa//61%//P98196
BRTHA3000296
BRTHA3003000//apoptosis-associated tyrosine kinase [Homo sapiens]//1.00E−161
//300aa//64%//NM_004920
BRTHA3008826
CERVX2002013//TRANSCRIPTION FACTOR BTEB2 (BASIC TRANSCRIPTION ELEMENT
BINDING PROTEIN 2) (GC BOX BINDING PROTEIN 2).//3.30E−33//81aa//81%//
Q13887
CTONG1000113//ZINC FINGER PROTEIN 184 (FRAGMENT).//0//641aa//85%//Q99676
CTONG2003348//Mus musculus mRNA for OASIS protein, complete cds.//9.60E−87
//392aa//51%//AB017614
CTONG2004000
CTONG2008721//Homo sapiens CAGH44 mRNA, partial cds.//2.70E−94//215aa//
91%//U80741
CTONG2015596
CTONG2015633
CTONG2016942//Homo sapiens serine protease DESC1 (DESC1) mRNA, complete
cds.//2.00E−95//425aa//43%//AF064819
CTONG2019822
CTONG2020374
CTONG2020378//ZINC FINGER PROTEIN 35 (ZFP-35).//5.70E−100//322aa//55%//
P15620
CTONG2020411//BASONUCLIN.//1.30E−139//616aa//48%//Q01954
CTONG2020974//Homo sapiens mRNA for putative progesterone binding protein.//
8.10E−118//223aa//100%//AJ002030
CTONG2024031
CTONG2028758//Mus musculus zfh-4 mRNA for zinc-finger homeodomain protein 4,
complete cds.//0//907aa//91%//AB024499
CTONG3001501//Mus musculus glucocorticoid-induced gene 1 mRNA, complete
cds.//3.60E−202//413aa//89%//AF292939
CTONG3002552
CTONG3003598
CTONG3004550//SH3-domain binding protein 4 [Homo sapiens]//1.00E−179//
351aa//42%//NM_014521
CTONG3004726//ZINC FINGER PROTEIN 91 (ZINC FINGER PROTEIN HTF10) (HPF7).//
4.20E−230//663aa//55%//Q05481
CTONG3009287
DFNES2011192//Homo sapiens ZNF140-like transcription factor mRNA, complete
cds.//1.10E−221//396aa//99%//AF326206
FCBBF1000509//Homo sapiens mRNA for photolyase, complete cds.//9.80E−280//
500aa//99%//D83702
FCBBF3010361//Danio rerio mRNA for winged helix nude (whn gene).//2.10E−107
//336aa//64%//AJ252024
FCBBF3027854//contains similarity to Arabidopsis thaliana DNA-damage-
repair/tolerance resistance protein DRT111 (SW: P42698//9.60E−12//87aa//
37%//AAC13593
FEBRA2000790
FEBRA2001990//SON OF SEVENLESS PROTEIN HOMOLOG 2 (SOS-2) (MSOS-2)
(FRAGMENT).//4.00E−09//175aa//20%//Q02384
FEBRA2006519//Mus musculus papilin mRNA, complete cds.//4.90E−58//327aa//
37%//AF314171
FEBRA2008692//Homo sapiens IRE1b mRNA for protein kinase/ribonuclease IRE1
beta, complete cds.//0//926aa//96%//AB047079
FEBRA2014122//wizL [Mus musculus]//0//661aa//88%//BAA32790
FEBRA2027609//ZINC FINGER PROTEIN 91 (ZINC FINGER PROTEIN HTF10) (HPF7).//
5.60E−196//688aa//51%//Q05481
FEBRA2028256//FIBRILLIN 2 PRECURSOR.//2.90E−231//527aa//62%//Q61555
FEBRA2028516
HCASM2002754
HCASM2003018//Homo sapiens nuclear transcription factor NFX2 (NFX2) mRNA,
complete cds.//3.90E−70//666aa//31%//AF332009
HCASM2003099//HISTONE DEACETYLASE CLR3.//5.00E−19//108aa//49%//P56523
HCASM2003357
HCASM2008536//DNA-REPAIR PROTEIN XRCC1.//2.00E−12//119aa//36%//P18887
HCASM2009424//regulatory factor (trans-acting) 2 [Mus musculus]//7.00E−24//
60aa//48%//NP_033082
HCHON2000508//Homo sapiens prostate antigen PARIS-1 mRNA, complete cds.//
0//686aa//99%//AY026527
HCHON2000743
HCHON2004858//Zinc finger protein 29 (Zfp-29).//1.00E−124//208aa//60%//
Q07230
HEART2009680//Vasoactive intestinal polypeptide receptor 2 precursor (VIP-R-
2) (Pituitary adenylate cyclase activating polypeptide type III receptor)
(PACAP type III receptor) (PACAP-R-3) (Helodermin-preferring VIP receptor).//
0//309aa//96%//P41587
HLUNG2013350//Mus musculus mRNA for synaptotagmin VIII, complete cds.//
1.70E−39//126aa//67%//AB026805
HLUNG2015418//similar to cadherin and Drosophila Fat protein; similar to
CAA60685 (PID: g1107687) [Homo sapiens].//1.00E−139//500aa//91%//AAD28068
HLUNG2015548//INOSINE-5′ - MONOPHOSPHATE DEHYDROGENASE 1 (EC 1.1.1.205) (IMP
DEHYDROGENASE 1) (IMPDH-I) (IMPD 1).//3.30E−253//521aa//94%//P20839
HLUNG2016862
HSYRA2005628//ZINC FINGER PROTEIN 195.//1.30E−237//426aa//78%//014628
IMR322001879
IMR322007078
IMR322008651//DNA REPAIR PROTEIN RAD51 HOMOLOG 1.//8.40E−154//340aa//
89%//Q06609
IMR322013396//Homo sapiens cone photoreceptor cGMP-gated channel alpha
subunit (CNGA3) mRNA, complete cds.//0//638aa//98%//AF065314
IMR322013731//TAT-BINDING HOMOLOG 7.//1.90E−74//187aa//47%//P54816
LIVER2000247//RENAL SODIUM/DICARBOXYLATE COTRANSPORTER (NA (+)/DICARBOXYLATE
COTRANSPORTER).//2.70E−57//243aa//48%//Q13183
MESAN2001770//EUKARYOTIC TRANSLATION INITIATION FACTOR 4 GAMMA (EIF-4-GAMMA)
(EIF-4G) (EIF4G) (P220).//4.70E−185//420aa//86%//Q04637
MESAN2005303//DNA BINDING PROTEIN URE-B1 (EC 6.3.2.—).//2.80E−36//171aa//
46%//P51593
MESAN2014412//Mus musculus zfh-4 mRNA for zinc-finger homeodomain protein 4,
complete cds.//2.8e−317//606aa//92%//AB024499
MESAN2015501//ZINC FINGER PROTEIN 91 (ZINC FINGER PROTEIN HTF10) (HPF7).//
7.80E−222//753aa//52%//Q05481
NT2RI2005772//Rattus norvegicus mRNA for DLG6 alpha, complete cds.//4.30E−176
//273aa//75%//AB030499
NT2RI2008952//ZINC FINGER PROTEIN 84 (ZINC FINGER PROTEIN HPF2).//1.40E−131
//393aa//55%//P51523
NT2RI2009583//Homo sapiens orphan G-protein coupled receptor (GPRC5C) mRNA,
complete cds.//3.70E−241//441aa//100%//AF207989
NT2RI2018448//AE-binding protein 2 [Mus musculus]//1.00E−153//258aa//
92%//NM_009637
NT2RI2027157//Mouse SDR2 mRNA, complete cds.//3.20E−233//539aa//77%//
D50464
NT2RI3000174//Homo sapiens HepA-related protein HARP mRNA, complete cds.//
1.00E−38//136aa//46%//NP_054859.1
NT2RI3001132//Mus musculus Arkadia (Arkadia) mRNA, complete cds.//0//
994aa//90%//AF330197
NT2RI3002557
NT2RI3005928
NT2RI3007167
NT2RI3007443//MITOGEN-ACTIVATED PROTEIN KINASE KINASE KINASE 5 (EC 2.7.1.—)
(MAPK/ERK KINASE KINASE 5) (MEK KINASE 5) (MEKK 5) (APOPTOSIS SIGNAL-
REGULATING KINASE 1) (ASK-1).//9.50E−244//606aa//66%//Q99683
NT2RP7008435//EPITHIN (EC 3.4.21.—).//6.60E−79//354aa//43%//P56677
NT2RP8000521
NTONG2008093//6PF-2-K/FRU-2, 6-P2ASE BRAIN/PLACENTA-TYPE ISOZYME [INCLUDES:
6-PHOSPHOFRUCTO-2-KINASE (EC 2.7.1.105); FRUCTOSE-2, 6-BISPHOSPHATASE (EC
3.1.3.46)].//1.60E−72//143aa//97%//Q16875
OCBBF2003327//ADAM-TS 6 precursor (EC 3.4.24.—) (A disintegrin and
metalloproteinase with thrombospondin motifs 6) (ADAMTS-6) (ADAM-TS6).//
2.00E−63//108aa//51%//Q9UKP5
OCBBF2005433//N-CHIMAERIN (NC) (N-CHIMERIN) (ALPHA CHIMERIN) (A-
CHIMAERIN).//7.70E−24//213aa//33%//P30337
OCBBF2006987
OCBBF2008144//ZINC FINGER PROTEIN 91 (ZINC FINGER PROTEIN HTF10) (HPF7).//
5.50E−244//757aa//56%//Q05481
OCBBF2009583//ZINC FINGER PROTEIN 184 (FRAGMENT).//1.20E−75//345aa//36%//
Q99676
OCBBF2011669//Zinc finger protein 91 (Zinc finger protein HTF10) (HPF7).//
1.00E−90//222aa//26%//Q05481
OCBBF2019684//ZINC FINGER PROTEIN 29 (ZFP-29).//6.90E−246//465aa//90%//
Q07230
OCBBF2020048//95 kDa retinoblastoma protein binding protein; KIAA0661 gene
product//9.90E−97//339aa//63%//XP_010492
OCBBF2024284//GUANINE NUCLEOTIDE-BINDING PROTEIN BETA SUBUNIT-LIKE PROTEIN
12.3 (P205) (RECEPTOR OF ACTIVATED PROTEIN KINASE C 1) (RACK1).//1.60E−72//
135aa//99%//P25388
OCBBF2030116
OCBBF2032274//Zinc finger protein 93 (Zinc finger protein HTF34)
(Fragment).//1.00E−145//247aa//67%//P35789
OCBBF2034637//microtubule associated testis specific serine/threonine
protein kinase [Mus musculus]//0//450aa//79%//NP_032667
OCBBF3000167//ZINC FINGER PROTEIN 83 (ZINC FINGER PROTEIN HPF1).//5.00E−156
//405aa//62%//P51522
OCBBF3002654//Triple functional domain protein (PTPRF interacting
protein).//0//510aa//93%//O75962
OCBBF3003761
OCBBF3004972
PERIC2007068//Mus musculus mRNA for 1A13 protein.//3.30E−121//385aa//
62%//X83587
PLACE7000333
PLACE7000502//Human Notch4 (hNotch4) mRNA, complete cds.//0//666aa//99%//
U95299
PROST2000452//TRANSMEMBRANE PROTEASE, SERINE 2 (EC 3.4.21.—).//1.60E−47//
186aa//50%//O15393
PROST2009320//enigma protein; LIM domain protein [Homo sapiens]//6.00E−42//
77aa//71%//NM_005451
PROST2019487
PUAEN2006335
SKMUS2003194
SPLEN2004611//Homo sapiens transcription factor 7-like 2 (T-cell specific,
HMG-box) (TCF7L2),//1.00E−19//50aa//47%//NM_030756
SPLEN2016135//Zinc finger protein 2 (Zfp-2) (mKR2 protein).//6.00E−27//
64aa//39%//P08043
SPLEN2016781//zinc finger transcription factor REST protein//5.00E−18//
80aa//33%//AAB94893
SPLEN2016932
SPLEN2030847//Mus musculus Kif21b (Kif21b) mRNA, complete cds.//1.00E−85//
110aa//94%//AF202893
SPLEN2033490
SPLEN2036702//TENSIN.//8.30E−175//409aa//54%//Q04205
SPLEN2037319
SPLEN2039311//Pro-Pol-dUTPasepolyprotein.*//2.70E−15//93aa//53%//O02711*
SPLEN2039379//Homo sapiens phosphatidylinositol 4-kinase 230 (pi4K230) mRNA,
complete cds.//0//645aa//99%//AF012872
STOMA2003158//DEOXYRIBONUCLEASE GAMMA PRECURSOR (EC 3.1.21.—) (DNASE GAMMA)
(DEOXYRIBONUCLEASE I-LIKE 3) (DNASE I HOMOLOGOUS PROTEIN DHP2) (LIVER AND
SPLEEN DNASE) (LS-DNASE) (LSD).//5.40E−143//267aa//99%//Q13609
STOMA2004893
SYNOV1000256//Human preferentially expressed antigen of melanoma (PRAME)
mRNA, complete cds.//2.80E−29//275aa//35%//U65011
SYNOV2001660
SYNOV2006620
SYNOV2013637//Homo sapiens putative renal organic anion transporter 1
(hROAT1) mRNA, complete cds.//2.40E−54//213aa//53%//AF057039
SYNOV2021953//hematopoietic zinc finger [Mus musculus]//1.00E−63//300aa//
51%//NP_038894
SYNOV4002744//Zinc finger protein 84 (Zinc finger protein HPF2).//2.00E−26
//75aa//30%//P51523
SYNOV4003981//megakaryocyte stimulating factor [Homo sapiens]//0//400aa//
74%//NM_005807
SYNOV4005739
SYNOV4005889
TBAES2000932//sirtuin 7; sirtuin type 7; silent mating type information
regulation 2, S. cerevisiae, homolog 7; sir2-related protein type 7 [Homo
sapiens]//1.00E−69//120aa//93%//NM_016538
TESOP2000390//Mitogen-activated protein kinase kinase kinase 14 (EC
2.7.1.37) (NF-kappa beta-inducing kinase) (Serine/threonine protein kinase
NIK) (HsNIK).//0//354aa//81%//Q99558
TESOP2001796//RING finger protein 19 (Dorfin) (Double ring-finger protein)
(p38 protein).//0//320aa//69%//Q9NV58
TESOP2005199//ZINC FINGER PROTEIN 7 (ZINC FINGER PROTEIN KOX4) (ZINC FINGER
PROTEIN HF.16).//8.40E−104//317aa//49%//P17097
TESOP2006398//zinc finger-like; similar to P52742 (PID: g1731411) [Homo
sapiens].//4.00E−22//100aa//97%//AAC36300
TESOP2006865//ZINC FINGER PROTEIN MFG-3.//5.30E−29//116aa//55%//P16374
TESOP2007384//Mus musculus putative purine nucleotide binding protein mRNA,
complete cds.//1.00E−78//288aa//55%//U44731
TESTI1000266
TESTI2008901
TESTI2015626//Human mRNA for phosphoribosypyrophosphate synthetase-
associated protein 39, complete cds.//1.00E−181//356aa//99%//D61391
TESTI2025924//CASEIN KINASE I, EPSILON ISOFORM (EC 2.7.1.—) (CKI-EPSILON).//
1.80E−42//265aa//36%//P49674
TESTI2026647//PROBABLE ATP-DEPENDENT RNA HELICASE DDX10 (DEAH BOX PROTEIN
10).//6.40E−48//226aa//45%//Q13206
TESTI2029252//Homo sapiens mRNA for LAK-4p, complete cds.//2.40E−79//
410aa//38%//AB002405
TESTI2032643//ADENYLATE CYCLASE, TYPE IV (EC 4.6.1.1) (ATP PYROPHOSPHATE-
LYASE) (ADENYLYL CYCLASE).//1.00E−176//155aa//75%//P26770
TESTI2034251//ZINC FINGER PROTEIN 91 (ZINC FINGER PROTEIN HTF10) (HPF7).//
3.10E−186//546aa//57%//Q05481
TESTI2035981
TESTI2036288//PROSTAGLANDIN-F SYNTHASE 1 (EC 1.1.1.188) (PGF SYNTHASE 1)
(PGF 1) (PROSTAGLANDIN-D2 11 REDUCTASE 1) (PGFSI).//1.10E−41//120aa//69%//
P05980
TESTI2037830
TESTI2039060//MALTASE-GLUCOAMYLASE, INTESTINAL [INCLUDES: MALTASE (EC
3.2.1.20) (ALPHA-GLUCOSIDASE); GLUCOAMYLASE (EC 3.2.1.3) (GLUCAN 1,4-ALPHA-
GLUCOSIDASE)].//8.60E−287//641aa//77%//O43451
TESTI2049956//Vegetatible incompatibility protein HET-E-1.//5.00E−43//
107aa//34%//Q00808
TESTI2050780//organic anion transporter OATP-E [Homo sapiens]//4.00E−28//
71aa//34%//NM_016354
TESTI4000137
TESTI4000155
TESTI4000183//ZINC FINGER PROTEIN 177.//4.60E−66//299aa//43%//Q13360
TESTI4000214//similar to human transcription factor TFIIS (S34159)//7.00E−40
//400aa//32%//BAA13438
TESTI4000319//GUANINE NUCLEOTIDE DISSOCIATION STIMULATOR RALGDS FORM B
(RALGEF).//4.70E−50//256aa//48%//Q03386
TESTI4001984
TESTI4005317//Homo sapiens rab6 GTPase activating protein (GAP and
centrosome-associated) (GAPCENA), mRNA//0//536aa//95%//NM_012197
TESTI4006473//ATP-dependent RNA helicase A (Nuclear DNA helicase II) (NDH
II) (DEAD-box protein 9).//5.00E−72//208aa//33%//Q28141
TESTI4008058
TESTI4008302//zinc finger protein [Homo sapiens]//0//382aa//89%//
NM_014347
TESTI4010382//cytoplasmic dynein heavy chain 2 [Rattus norvegicus]//0//
1171aa//90%//NM_023024
TESTI4011070//Glucoamylase S1/S2 precursor (EC 3.2.1.3) (Glucan 1,4-alpha-
glucosidase) (1,4-alpha-D-glucan glucohydrolase).//5.00E−18//148aa//20%//
P08640
TESTI4011072//tudor domain containing 1 [Mus musculus]//1.00E−38//201aa//
22%//NM_031387
TESTI4011829
TESTI4013365//S-antigen protein precursor.//6.00E−07//109aa//20%//Q03400
TESTI4013602
TESTI4013894//mitsugumin 29 [Mus musculus]//1.00E−117//200aa//92%//
NM_008596
TESTI4014801//novel protein similar to archaeal, yeast and worm N2,N2-
dimethylguanosine tRNA methyltransferase [Homo sapiens]//0//312aa//89%//
NM_030934
TESTI4015012
TESTI4015442//zinc finger homeodomain 4 [Mus musculus]//0//875aa//79%//
NM_030708
TESTI4017714//Protease II (EC 3.4.21.83) (Oligopeptidase B).//4.00E−52//
175aa//25%//Q59536
TESTI4019657//cyclin G associated kinase [Homo sapiens]//1.00E−138//
311aa//88%//NP_005246
TESTI4021482//RAC-alpha serine/threonine kinase (EC 2.7.1.—) (RAC-PK-alpha)
(Protein kinase B) (PKB) (C-AKT).//3.00E−28//58aa//98%//P31749
TESTI4024387//RAB GDP dissociation inhibitor alpha (RAB GDI alpha) (GDI-
1).//1.00E−102//179aa//98%//P50398
TESTI4025268//77 kDa echinoderm microtubule-associated protein.//1.00E−45//
119aa//31%//Q26613
TESTI4025494//Zinc finger protein 33A (Zinc finger protein KOX31)
(HA0946).//0//384aa//49%//Q06730
TESTI4025547//double-stranded RNA-binding zinc finger protein JAZ [Homo
sapiens]//1.00E−161//279aa//87%//NM_012279
TESTI4025865
TESTI4026207//Kinesin-like protein KLP1.//1.00E−83//167aa//43%//P46870
TESTI4028938//Zinc finger protein 85 (Zinc finger protein HPF4) (HTF1).//
0//373aa//66%//Q03923
TESTI4028958
TESTI4029348
TESTI4029528
TESTI4029690
TESTI4031745//Mitogen-activated protein kinase kinase kinase 5 (EC 2.7.1.—)
(MAPK/ERK kinase kinase 5) (MEK kinase 5) (MEKK 5) (Apoptosis signal-
regulating kinase 1) (ASK-1).//0//447aa//58%//Q99683
TESTI4032090
TESTI4032112//Homo sapiens general transcription factor IIIC, polypeptide 1
(alpha subunit, 220 kD) (GTF3C1), mRNA//1.00E−121//213aa//82%//NM_001520
TESTI4036767
TESTI4038721
TESTI4041086
TESTI4046240//sirtuin 7//1.00E−115//44aa//98%//NP_057622
THYMU2004139//WEE1-LIKE PROTEIN KINASE (EC 2.7.1.112).//2.80E−137//534aa//
54%//P47817
THYMU2004284//UBIQUITIN-ACTIVATING ENZYME E1 (A1S9 PROTEIN).//9.00E−63//
122aa//100%//P22314
THYMU2006001//ZINC-BINDING PROTEIN A33.//8.40E−51//476aa//27%//Q02084
THYMU2028739//ZINC FINGER PROTEIN 263 (ZINC FINGER PROTEIN FPM315).//1.10E−39
//291aa//40%//O14978
THYMU2030462
THYMU2031139//Homo sapiens zinc metalloprotease ADAMTS7 (ADAMTS7) mRNA,
complete cds.//3.30E−105//504aa//42%//AF140675
THYMU2031249//SERINE/THREONINE-PROTEIN KINASE PCTAIRE-1 (EC 2.7.1.—).//
1.20E−138//291aa//92%//Q00536
THYMU2032976
THYMU2033401
THYMU2034279
THYMU2035078//LRP16 protein [Homo sapiens]//3.00E−50//80aa//55%//
NP_054786
THYMU2035710//Tyrosine-protein kinase-like 7 [Precursor]//1.00E−50//
121aa//80%//Q13308
THYMU2040925//CDP-DIACYLGLYCEROL-INOSITOL 3-PHOSPHATIDYLTRANSFERASE (EC
2.7.8.11) (PHOSPHATIDYLINOSITOL SYNTHASE) (PTDINS SYNTHASE) (PI SYNTHASE).//
4.60E−50//118aa//86%//O14735
THYMU3000269//SUCCINATE DEHYDROGENASE [UBIQUINONE] FLAVOPROTEIN SUBUNIT,
MITOCHONDRIAL PRECURSOR (EC 1.3.5.1) (FP) (FLAVOPROTEIN SUBUNIT OF COMPLEX
II).//2.80E−130//245aa//99%//P31040
THYMU3000360
THYMU3001428
TKIDN2008778
TKIDN2012771//DPY-19 PROTEIN.//5.30E−49//314aa//35%//P34413
TKIDN2018926
TLIVE2001684//complement component 3 precursor [Homo sapiens]//5.00E−59//
110aa//32%//NP_000055
TLIVE2002046//DELTA-AMINOLEVULINIC ACID DEHYDRATASE (EC 4.2.1.24)
(PORPHOBILINOGEN SYNTHASE) (ALADH).//3.10E−156//317aa//93%//P13716
TLIVE2007607//CYTOCHROME P450 4A4 (EC 1.14.14.1) (CYPIVA4) (PROSTAGLANDIN
OMEGA-HYDROXYLASE) (P450-P-2).//6.90E−127//448aa//49%//P10611
TRACH1000212
TRACH2000862//Mus musculus putative purine nucleotide binding protein mRNA,
complete cds.//5.40E−224//619aa//68%//U44731
TRACH2007483//GASTRULA ZINC FINGER PROTEIN XLCGF7.1 (FRAGMENT).//6.30E−12//
98aa//37%//P18735
TRACH2019672//Rattus norvegicus mRNA for 45 kDa secretory protein,
partial.//5.80E−163//303aa//96%//AJ132352
TRACH2024408//ankyrin 2, isoform 2; ankyrin-2, nonerythrocytic; ankyrin-B;
ankyrin, brain; ankyrin, neuronal; ankyrin, nonerythroid [Homo sapiens]//
1.00E−06//119aa//20%//NM_020977
TRACH2024559//Homo sapiens myosin 5c (MYO5C) mRNA, complete cds.//1.70E−59
//151aa//86%//AF272390
TRACH3000134//ZINC FINGER PROTEIN 84 (ZINC FINGER PROTEIN HPF2).//2.30E−215
//733aa//53%//P51523
TRACH3000420//Homo sapiens ATP-binding cassette transporter family A member
12 (ABCA12) mRNA, complete cds.//7.00E−253//745aa//41%//AY033486
TRACH3002561//UNR protein.//2.00E−34//66aa//95%//P18395
TRACH3003683//Lactoperoxidase precursor (EC 1.11.1.7) (LPO) (Salivary
peroxidase) (SPO).//1.00E−39//77aa//86%//P22079
TRACH3003832//PHD finger protein 2 [Mus musculus]//1.00E−153//321aa//
39%//NM_011078
TRACH3007866//dipeptidyl peptidase 8; hypothetical protein FLJ20283 [Homo
sapiens]//1.00E−105//172aa//53%//NM_017743
TUTER2000057//Mus musculus AE-1 binding protein AEBP2 mRNA, complete cds.//
1.10E−26//60aa//93%//AF090326
UTERU2004299
UTERU2008040//Mus musculus mRNA for stac, complete cds.//3.30E−82//405aa//
47%//D86639
UTERU2011220
UTERU2019534//Golgi apparatus protein 1 [Homo sapiens]//5.10E−61//118aa//
100%//XP_012515
UTERU2021820//dipeptidylpeptidase VI [Homo sapiens]//8.00E−23//50aa//
89%//NP_001927
UTERU2028734//Mus musculus slp2-a mRNA for synaptotagmin-like protein 2-a
delta 2S-III, complete cds.//2.30E−216//464aa//86%//AB057762
UTERU2032279//47 KDA HEAT SHOCK PROTEIN PRECURS0R (COLLAGEN-BINDING PROTEIN
1) (COLLIGIN 1).//6.90E−95//207aa//93%//P29043
UTERU2033577//Homo sapiens mRNA for repressor protein, partial cds.//1.80E−65
//209aa//65%//D30612
UTERU2035978
UTERU3000402
UTERU3000738//Beta-adrenergic receptor kinase 1 (EC 2.7.1.126) (Beta-ARK-1)
(G-protein coupled receptor kinase 2).//5.00E−76//139aa//94%//P26817
UTERU3001053//zinc finger protein [Homo sapiens]//0//319aa//91%//
NM_018651
UTERU3014791
UTERU3015069
UTERU3015412
UTERU3017176
TESTI4038779//zinc finger protein RIN ZF [Rattus norvegicus]//3.00E−73//
190aa//36%//NM_024489

LENGTHY TABLE
The patent application contains a lengthy table section. A copy of the table is available in electronic form from the USPTO web site (http://seqdata.uspto.gov/?pageRequest=docDetail&DocID=US20070020637A1) An electronic copy of the table will also be available from the USPTO upon request and payment of the fee set forth in 37 CFR 1.19(b)(3).

What is claimed is:

1. A polynucleotide selected from the group consisting of:
(a) a polynucleotide comprising a protein-coding region of the nucleotide sequence according to any one of SEQ ID NOs: 1-2188 and SEQ ID NOs: 4377-4683;
(b) a polynucleotide comprising the nucleotide sequence encoding a polypeptide that comprises the amino acid sequence of any one of SEQ ID NOs: 2189-4376 and SEQ ID NOs: 4684-4990;
(c) a polynucleotide comprising a nucleotide sequence encoding a polypeptide, which comprises the amino acid sequence selected from SEQ ID NO: SEQ ID NOs: 2189-4376 and SEQ ID NOs: 4684-4990 wherein one or more amino acids have been substituted, deleted, inserted, and/or added, and which is functionally equivalent to the polypeptide comprising the selected amino acid sequence as described above;
(d) a polynucleotide which hybridizes to a polynucleotide comprising the nucleotide sequence selected from SEQ ID NOs: 1-2188 and SEQ ID NOs: 4377-4683, and which comprises the nucleotide sequence encoding a polypeptide functionally equivalent to a polypeptide encoded by the selected nucleotide sequence as described above;
(e) a polynucleotide comprising a nucleotide sequence encoding a partial amino acid sequence of a polypeptide encoded by the polynucleotides according to any one of (a)-(d);
(f) a polynucleotide comprising a nucleotide sequence having at least 70% identity to the nucleotide sequence of any one of SEQ ID NOs: 1-2188 and SEQ ID NOs: 4377-4683; and
(g) a polynucleotide comprising a nucleotide sequence having at least 90% identity to the nucleotide sequence of any one of SEQ ID NOs: 1-2188 and SEQ ID NOs: 4377-4683.

2. A polypeptide encoded by the polynucleotide according to claim 1, or a partial peptide thereof.

3. An antibody which binds to the polypeptide or the peptide according to claim 2.

4. An immunoassay method for the polypeptide or the peptide according to claim 2, which comprises the steps of:
(a) contacting the polypeptide or the peptide according to claim 2 with the antibody according to claim 3; and
(b) observing the binding between the two.

5. A vector comprising the polynucleotide according to claim 1.

6. A transformant comprising the polynucleotide according to claim 1 or the vector according to claim 5.

7. A transformant which comprises the polynucleotide according to claim 1 or vector according to claim 5 in an expressible manner.

8. A method for producing the polypeptide or the peptide according to claim 2, which comprises the steps of:
(a) culturing the transformant according to claim 7; and
(b) recovering the expression product.

9. An oligonucleotide comprising 15 or more nucleotides, which comprises the nucleotide sequence according to any one of SEQ ID NOs: 1-2188 and SEQ ID NOs: 4377-4683, or a nucleotide sequence complementary to the complementary strand thereof.

10. A primer for synthesizing a polynucleotide, which comprises the oligonucleotide according to claim 9.

11. A probe for detecting a polynucleotide, which comprises the oligonucleotide according to claim 9.

12. A polynucleotide according to any one of:
(a) an antisense polynucleotide comprising a nucleotide sequence complementary to the transcript of the polynucleotide according to claim 1;
(b) a polynucleotide with the ribozyme activity for specifically cleaving the transcript of the polynucleotide according to claim 1; and
(c) a polynucleotide which downregulates the expression of the polynucleotide of claim 1 due to RNAi activity in a host cell.

13. A method for detecting the polynucleotide according to claim 1, which comprises the steps of:
(a) incubating a target polynucleotide with the oligonucleotide according to claim 9 under conditions ensuring hybridization; and
(b) detecting the hybridization between the target polynucleotide and the oligonucleotide according to claim 9.

14. A database of polynucleotides and/or polypeptides, which comprises information on at least one of the nucleotide sequences of SEQ ID NOs: 1-2188 and SEQ ID NOs: 4377-4683 and/or on at least one of the amino acid sequences of SEQ ID NOs: 2189-4376 and SEQ ID NOs: 4684-4990.
